(19) United States
(12) Patent Application Publication
(10) Pub. No.: US 2025/0066390 A1
(43) Pub. Date: Feb. 27, 2025

(54) HETEROCYCLIC COMPOUND, ORGANIC LIGHT-EMITTING DEVICE INCLUDING HETEROCYCLIC COMPOUND, AND ELECTRONIC APPARATUS INCLUDING ORGANIC LIGHT-EMITTING DEVICE

(71) Applicants: Samsung Electronics Co., Ltd., Suwon-si (KR); Research & Business Foundation SUNGKYUNKWAN UNIVERSITY, Suwon-si (KR)

(72) Inventors: Soonok Jeon, Suwon-si (KR); Junyeob Lee, Seongnam-si (KR); Halim Lee, Seongnam-si (KR); Jihoon Kang, Suwon-si (KR); Inkoo Kim, Suwon-si (KR); Youngmo Sung, Suwon-si (KR); Joonghyuk Kim, Seoul (KR); Sangho Park, Anyang-si (KR); Soohwan Sul, Suwon-si (KR); Won-joon Son, Suwon-si (KR); Hyeonho Choi, Seoul (KR)

(21) Appl. No.: 18/903,362

(22) Filed: Oct. 1, 2024

Related U.S. Application Data
(63) Continuation of application No. 17/465,019, filed on Sep. 2, 2021.

(30) Foreign Application Priority Data
Feb. 22, 2021 (KR) 10-2021-0023357

(51) Int. Cl. C07D 519/00; C07D 487/14; H10K 50/11; H10K 50/12; H10K 71/00; H10K 85/60; H10K 101/00; H10K 101/10; H10K 101/30; H10K 101/40

(52) U.S. Cl. CPC C07D 519/00; C07D 487/14; H10K 85/6572; H10K 50/11; H10K 50/121; H10K 71/00; H10K 2101/10; H10K 2101/30; H10K 2101/40; H10K 2101/90

(57) ABSTRACT

Provided are a heterocyclic compound represented by Formula 1-1 or 1-2, an organic light-emitting device including the heterocyclic compound, and an electronic apparatus including the organic light-emitting device:

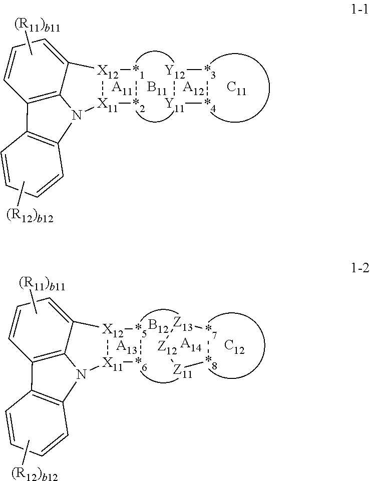

Formulae 1-1 and 1-2 may each be understood by referring to the descriptions of Formulae 1-1 and 1-2 provided herein.

### CROSS-REFERENCE TO RELATED APPLICATION

This application is a continuation of U.S. application Ser. No. 17/465,019, filed on Sep. 2, 2021, which claims priority under 35 U.S.C. § 119 to Korean Patent Application No. 10-2021-0023357, filed on Feb. 22, 2021, in the Korean Intellectual Property Office, the contents of which are incorporated by reference herein in their entireties.

### BACKGROUND

### 1. Field

The present disclosure relates to a heterocyclic compound, an organic light-emitting device including the heterocyclic compound, and an electronic apparatus including the organic light-emitting device.

### 2. Description of the Related Art

Organic light-emitting devices (OLEDs) are self-emissive devices that, as compared with conventional devices, have wide viewing angles, high contrast ratios, short response times, and excellent brightness, driving voltage, and response speed characteristics, and produce full-color images.

OLEDs include an anode, a cathode, and an organic layer between the anode and the cathode and including an emission layer. A hole transport region may be between the anode and the emission layer, and an electron transport region may be between the emission layer and the cathode. Holes provided from the anode may move toward the emission layer through the hole transport region, and electrons provided from the cathode may move toward the emission layer through the electron transport region. The holes and the electrons recombine in the emission layer to produce excitons. The excitons may transition from an excited state to a ground state, thus generating light.

### SUMMARY

One or more embodiments relate to a heterocyclic compound, an organic light-emitting device including the heterocyclic compound, and an electronic apparatus including the organic light-emitting device.

Additional aspects will be set forth in part in the description which follows and, in part, will be apparent from the description, or may be learned by practice of the presented embodiments of the disclosure.

According to an aspect, a heterocyclic compound may be represented by one of Formulae 11, 12, and 14 to 17:

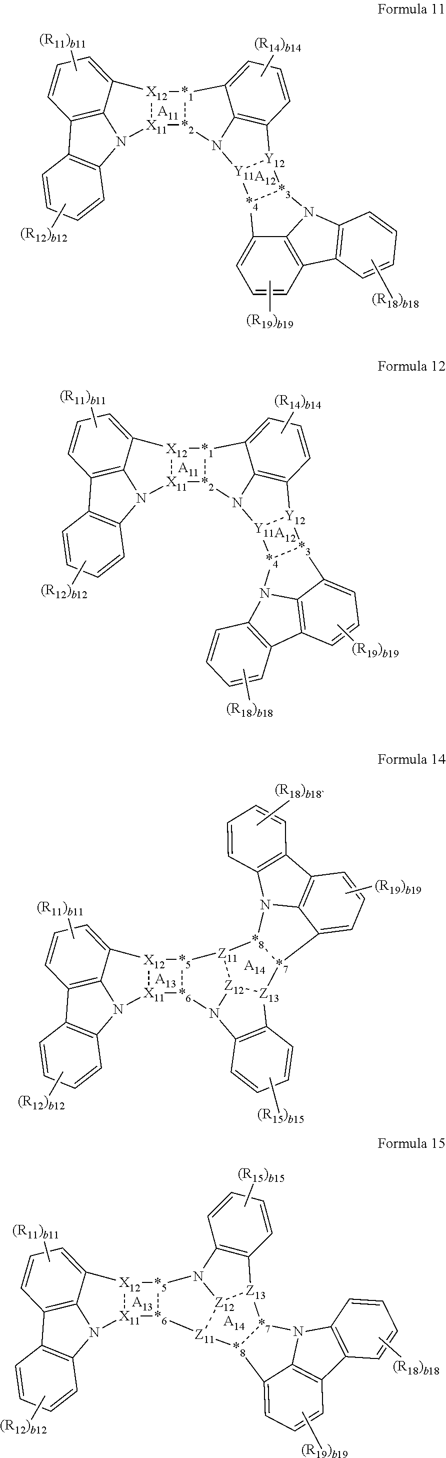

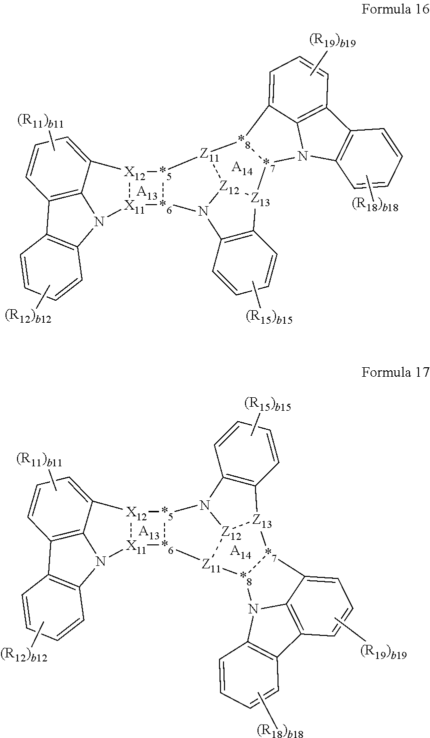

wherein, in Formulae 11, 12, and 14 to 17,

A₁₁ and A₁₃ are each independently a group represented by Formula 2-1,

A₁₂ is a group represented by Formula 2-2,

A₁₄ is a group represented by Formula 2-3,

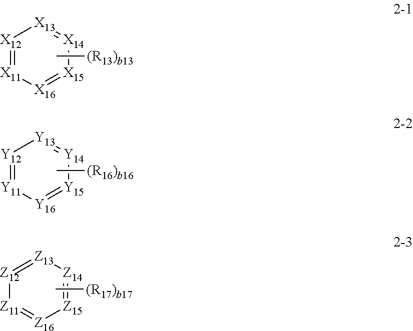

wherein, in Formulae 2-1 to 2-3,

X₁₁ to X₁₆, Y₁₁ to Y₁₆, and Z₁₁ to Z₁₆ are each independently a carbon atom, any suitable two adjacent groups X₁₃ to X₁₆ may be *1 and *2 in Formulae 11 and 12 or *5 and *6 in Formulae 14 to 17, and any suitable two adjacent groups Y₁₃ to Y₁₆ may be *3 and *4 in Formulae 11 and 12, and any suitable two adjacent groups Z₁₄ to Z₁₆ may be *7 and *8 in Formulae 14 to 17,

in Formula 11, ii)*1 is X₁₃, *2 is X₁₄, *3 is Y₁₄, and *4 is Y₁₅; or v)*1 is X₁₄, *2 is X₁₅, *3 is Y₁₄, and *4 is Y₁₅, in Formula 12, i)*1 is X₁₃, *2 is X₁₄, *3 is Y₁₃, and *4 is Y₁₄; ii) *1 is X₁₃, *2 is X₁₄, *3 is Y₁₄, and *4 is Y₁₅; or iv)*1 is X₁₄, *2 is X₁₅, *3 is Y₁₄, and *4 is Y₁₅, in Formula 14, i)*5 is X₁₃, *6 is X₁₄, *7 is Z₁₄, and *8 is Z₁₅; iii)*5 is X₁₄, *6 is X₁₅, *7 is Z₁₄, and *8 is Z₁₅; or iv)*5 is X₁₄, *6 is X₁₅, *7 is Z₁₅, and *8 is Z₁₆, in Formula 15, iii)*5 is X₁₄, *6 is X₁₅, *7 is Z₁₄, and *8 is Z₁₅; in Formula 16, iii)*5 is X₁₄, *6 is X₁₅, *7 is Z₁₄, and *8 is Z₁₅; or iv)*5 is X₁₄, *6 is X₁₅, *7 is Z₁₅, and *8 is Z₁₆, in Formula 17, iv)*5 is X₁₄, *6 is X₁₅, *7 is Z₁₅, and *8 is Z₁₆,

R₁₁ to R₁₉ are each independently hydrogen, deuterium, —F, —Cl, —Br, —I, —SF₅, a hydroxyl group, a cyano group, a nitro group, an amidino group, a hydrazino group, a hydrazono group, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₇-C₆₀ alkyl aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted C₂-C₆₀ alkyl heteroaryl group, a substituted or unsubstituted C₁-C₆₀ heteroaryloxy group, a substituted or unsubstituted C₁-C₆₀ heteroarylthio group, a substituted or unsubstituted monovalent aromatic condensed polycyclic group, a substituted or unsubstituted monovalent aromatic condensed heteropolycyclic group, a substituted or unsubstituted monovalent non-aromatic condensed polycyclic group, a substituted or unsubstituted monovalent non-aromatic condensed heteropolycyclic group, —Si(Q₁)(Q₂)(Q₃), —Ge(Q₁)(Q₂)(Q₃), —C(Q₁)(Q₂)(Q₃), —B(Q₁)(Q₂), —N(Q₁)(Q₂), —P(Q₁)(Q₂), —C(═O)(Q₁), —S(═O)(Q₁), —S(═O)₂(Q₁), —P(═O)(Q₁)(Q₂), or —P(═S)(Q₁)(Q₂),

b11, b14, and b19 are each independently 0, 1, 2, or 3,

b12, b15, and b18 are each independently 0, 1, 2, 3, or 4,

b13 and b16 are each independently 0, 1, or 2, and b17 is 0 or 1,

wherein Q₁ to Q₃ are each independently hydrogen, deuterium, —F, —Cl, —Br, —I, a hydroxyl group, a cyano group, a nitro group, an amidino group, a hydrazino group, a hydrazono group, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₇-C₆₀ alkyl aryl group, a C₆-C₆₀ aryloxy group, a C₆-C₆₀ arylthio group, a C₁-C₆₀ heteroaryl group, a C₂-C₆₀ alkyl heteroaryl group, a C₁-C₆₀ heteroaryloxy group, a C₁-C₆₀ heteroarylthio group, a monovalent aromatic condensed polycyclic group, a monovalent aromatic condensed heteropolycyclic group, a monovalent non-aromatic condensed polycyclic group, a monovalent non-aromatic condensed heteropolycyclic group, a C₁-C₆₀ alkyl group substituted with at least one deuterium, —F, a cyano group, a C₁-C₆₀ alkyl group, and a C₆-C₆₀ aryl group, and a C₆-C₆₀ aryl group substituted with at least one deuterium, —F, a cyano group, a C₁-C₆₀ alkyl group, or a C₆-C₆₀ aryl group.

According to one or more embodiments, an organic light-emitting device may include a first electrode, a second electrode, and an organic layer between the first electrode and the second electrode and including an emission layer, and the organic light-emitting device may include the heterocyclic compound.

According to an aspect of another embodiment, an electronic apparatus may include the organic light-emitting device.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 illustrates a schematic view of an organic light-emitting device 1 according to an exemplary embodiment;

FIGS. 2A-2E illustrate the general energy transfer of the First Embodiment (FIG. 2A), the Second Embodiment (FIG. 2B), the Third Embodiment (FIG. 2C), the Fourth Embodiment (FIG. 2D), and the Fifth Embodiment (FIG. 2E); and

FIG. 3 illustrates a schematic view of an organic light-emitting device 100 according to an exemplary embodiment.

FIG. 4 is a schematic view of an organic light-emitting device 200 according to an exemplary embodiment.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects. As used herein, the term “and/or” includes any and all combinations of one or more of the associated listed items. Expressions such as “at least one of,” when preceding a list of elements, modify the entire list of elements and do not modify the individual elements of the list.

It will be understood that when an element is referred to as being “on” another element, it can be directly on the other element or intervening elements may be present therebetween In contrast, when an element is referred to as being “directly on” another element, there are no intervening elements present

It will be understood that, although the terms “first,” “second,” “third” etc. may be used herein to describe various elements, components, regions, layers and/or sections, these elements, components, regions, layers and/or sections should not be limited by these terms These terms are only used to distinguish one element, component, region, layer or section from another element, component, region, layer or section Thus, “a first element,” “component,” “region,” “layer” or “section” discussed below could be termed a second element, component, region, layer or section without departing from the teachings herein.

The terminology used herein is for the purpose of describing particular embodiments only and is not intended to be limiting. As used herein, “a,” “an,” “the,” and “at least one” do not denote a limitation of quantity, and are intended to cover both the singular and plural, unless the context clearly indicates otherwise. For example, “an element” has the same meaning as “at least one element,” unless the context clearly indicates otherwise.

“Or” means “and/or.” As used herein, the term “and/or” includes any and all combinations of one or more of the associated listed items It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

Furthermore, relative terms, such as “lower” or “bottom” and “upper” or “top,” may be used herein to describe one element's relationship to another element as illustrated in the Figures It will be understood that relative terms are intended to encompass different orientations of the device in addition to the orientation depicted in the Figures For example, if the device in one of the figures is turned over, elements described as being on the “lower” side of other elements would then be oriented on “upper” sides of the other elements The exemplary term “lower,” can therefore, encompasses both an orientation of “lower” and “upper,” depending on the particular orientation of the figure Similarly, if the device in one of the figures is turned over, elements described as “below” or “beneath” other elements would then be oriented “above” the other elements The exemplary terms “below” or “beneath” can, therefore, encompass both an orientation of above and below.

“About” or “approximately” as used herein is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” can mean within one or more standard deviations, or within ±30%, 20%, 10% or 5% of the stated value.

Unless otherwise defined, all terms (including technical and scientific terms) used herein have the same meaning as commonly understood by one of ordinary skill in the art to which this disclosure belongs It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

Exemplary embodiments are described herein with reference to cross section illustrations that are schematic illustrations of idealized embodiments As such, variations from the shapes of the illustrations as a result, for example, of manufacturing techniques and/or tolerances, are to be expected Thus, embodiments described herein should not be construed as limited to the particular shapes of regions as illustrated herein but are to include deviations in shapes that result, for example, from manufacturing. For example, a region illustrated or described as flat may, typically, have rough and/or nonlinear features Moreover, sharp angles that are illustrated may be rounded Thus, the regions illustrated in the figures are schematic in nature and their shapes are not intended to illustrate the precise shape of a region and are not intended to limit the scope of the present claims.

The heterocyclic compound may be represented by Formula 1-1 or 1-2:

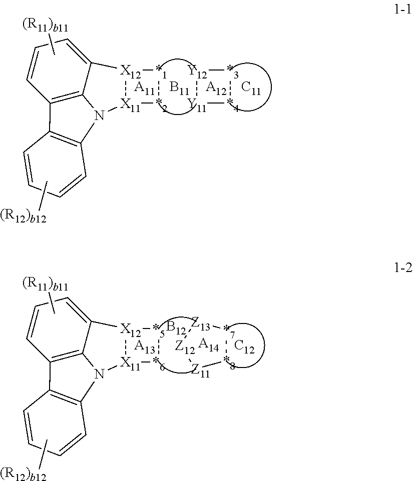

wherein, in Formulae 1-1 and 1-2, A₁₁ and A₁₃ may each independently be a group represented by Formula 2-1, A₁₂ may be a group represented by Formula 2-2, and A₁₄ may be a group represented by Formula 2-3:

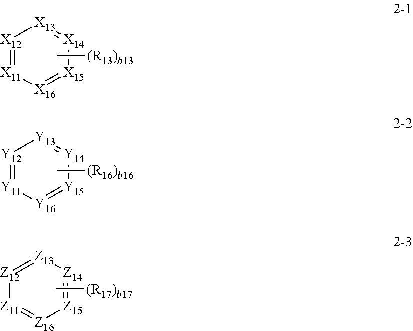

wherein, in Formula 2-1 to 2-3, X₁₁ to X₁₆, Y₁₁ to Y₁₆, and Z₁₁ to Z₁₆ may each independently be a carbon atom, any suitable two adjacent groups X₁₃ to X₁₆ may be *1 and *2 in Formula 1-1 or *5 and *6 in Formula 1-2, and any suitable two adjacent groups Y₁₃ to Y₁₆ may be *3 and *4 in Formula 1-1, and any suitable two adjacent groups Z₁₄ to Z₁₆ may be *7 and *8 in Formula 1-2.

In Formula 1-1, B₁₁ may be a group represented by Formula 2-4:

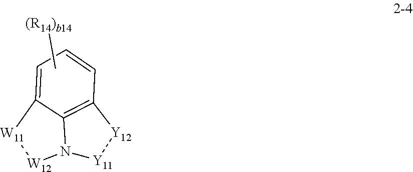

wherein, in Formula 2-4, W₁₁ and W₁₂ each independently be a carbon atom, and W₁₁ and W₁₂ may respectively be *1 and *2 in Formula 1-1.

In Formula 1-2, B₁₂ may be a group represented by Formula 2-5:

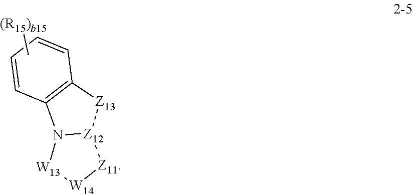

In Formula 2-5, W₁₃ and W₁₄ may each independently be a carbon atom, and W₁₃ and W₁₄ may respectively be *5 and *6 in Formula 1-2.

In Formulae 1-1 and 1-2, C₁₁ and C₁₂ may each independently be a group represented by Formula 2-6:

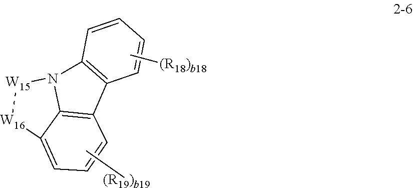

wherein, in Formula 2-6, W₁₅ and W₁₆ may each independently be a carbon atom, and W₁₅ and W₁₆ may respectively be *3 and *4 in Formula 1-1 or *7 and *8 in Formula 1-2.

In Formulae 1-1 and 1-2, R₁₁ to R₁₉ may each independently be hydrogen, deuterium, —F, —Cl, —Br, —I, —SF₅, a hydroxyl group, a cyano group, a nitro group, an amidino group, a hydrazino group, a hydrazono group, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₇-C₆₀ alkyl aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted C₂-C₆₀ alkyl heteroaryl group, a substituted or unsubstituted C₁-C₆₀ heteroaryloxy group, a substituted or unsubstituted C₁-C₆₀ heteroarylthio group, a substituted or unsubstituted monovalent aromatic condensed polycyclic group, a substituted or unsubstituted monovalent aromatic condensed heteropolycyclic group, a substituted or unsubstituted monovalent non-aromatic condensed polycyclic group, a substituted or unsubstituted monovalent non-aromatic condensed heteropolycyclic group, —Si(Q₁)(Q₂)(Q₃), —Ge(Q₁)(Q₂)(Q₃), —C(Q₁)(Q₂)(Q₃), —B(Q₁)(Q₂), —N(Q₁)(Q₂), —P(Q₁)(Q₂), —C(═O)(Q₁), —S(═O)(Q₁), —S(═O)₂(Q₁), —P(═O)(Q₁)(Q₂), or —P(═S)(Q₁)(Q₂),

wherein Q₁ to Q₃ may each independently be hydrogen, deuterium, —F, —Cl, —Br, —I, a hydroxyl group, a cyano group, a nitro group, an amidino group, a hydrazino group, a hydrazono group, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₇-C₆₀ alkyl aryl group, a C₆-C₆₀ aryloxy group, a C₆-C₆₀ arylthio group, a C₁-C₆₀ heteroaryl group, a C₂-C₆₀ alkyl heteroaryl group, a C₁-C₆₀ heteroaryloxy group, a C₁-C₆₀ heteroarylthio group, a monovalent aromatic condensed polycyclic group, a monovalent aromatic condensed heteropolycyclic group, a monovalent non-aromatic condensed polycyclic group, a monovalent non-aromatic condensed heteropolycyclic group, a C₁-C₆₀ alkyl group substituted with at least one deuterium, —F, a cyano group, a C₁-C₆₀ alkyl group, a C₆-C₆₀ aryl group, or any combination thereof, or a C₆-C₆₀ aryl group substituted with at least one deuterium, —F, a cyano group, a C₁-C₆₀ alkyl group, a C₆-C₆₀ aryl group, or any combination thereof.

In some embodiments, in Formulae 1-1 and 1-2, R₁₁ to R₁₉ may each independently be: hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, —SF₅, a C₁-C₂₀ alkyl group, a C₁-C₂₀ alkenyl group, a C₁-C₂₀ alkoxy group, or a C₁-C₂₀ alkylthio group;

a C₁-C₂₀ alkyl group, a C₁-C₂₀ alkenyl group, a C₁-C₂₀ alkoxy group, or a C₁-C₂₀ alkylthio group, each substituted with deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₁₀ alkyl group, a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a bicyclo[1.1.1]pentyl group, a bicyclo[2.1.1]hexyl group, a bicyclo[2.2.1]heptyl group, a bicyclo[2.2.2]octyl group, a (C₁-C₂₀ alkyl)cyclopentyl group, a (C₁-C₂₀ alkyl)cyclohexyl group, a (C₁-C₂₀ alkyl)cycloheptyl group, a (C₁-C₂₀ alkyl)cyclooctyl group, a (C₁-C₂₀ alkyl)adamantanyl group, a (C₁-C₂₀ alkyl)norbornanyl group, a (C₁-C₂₀ alkyl)norbornenyl group, a (C₁-C₂₀ alkyl)cyclopentenyl group, a (C₁-C₂₀ alkyl)cyclohexenyl group, a (C₁-C₂₀ alkyl)cycloheptenyl group, a (C₁-C₂₀ alkyl)bicyclo[1.1.1]pentyl group, a (C₁-C₂₀ alkyl)bicyclo[2.1.1]hexyl group, a (C₁-C₂₀ alkyl)bicyclo[2.2.1]heptyl group, a (C₁-C₂₀ alkyl)bicyclo[2.2.2]octyl group, a phenyl group, a (C₁-C₂₀ alkyl)phenyl group, a biphenyl group, a terphenyl group, a naphthyl group, a pyridinyl group, a pyrimidinyl group, or any combination thereof;

a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a bicyclo[1.1.1]pentyl group, a bicyclo[2.1.1]hexyl group, a bicyclo[2.2.1]heptyl group, a bicyclo[2.2.2]octyl group, a phenyl group, a (C₁-C₂₀ alkyl)phenyl group, a biphenyl group, a terphenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, an isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, an imidazopyrimidinyl group, an azacarbazolyl group, an azadibenzofuranyl group, or an azadibenzothiophenyl group, each unsubstituted or substituted with deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a deuterated C₂-C₂₀ alkyl group, a C₁-C₂₀ alkoxy group, a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a bicyclo[1.1.1]pentyl group, a bicyclo[2.1.1]hexyl group, a bicyclo[2.2.1]heptyl group, a bicyclo[2.2.2]octyl group, a (C₁-C₂₀ alkyl)cyclopentyl group, a (C₁-C₂₀ alkyl)cyclohexyl group, a (C₁-C₂₀ alkyl)cycloheptyl group, a (C₁-C₂₀ alkyl)cyclooctyl group, a (C₁-C₂₀ alkyl)adamantanyl group, a (C₁-C₂₀ alkyl)norbornanyl group, a (C₁-C₂₀ alkyl)norbornenyl group, a (C₁-C₂₀ alkyl)cyclopentenyl group, a (C₁-C₂₀ alkyl)cyclohexenyl group, a (C₁-C₂₀ alkyl)cycloheptenyl group, a (C₁-C₂₀ alkyl)bicyclo[1.1.1]pentyl group, a (C₁-C₂₀ alkyl)bicyclo[2.1.1]hexyl group, a (C₁-C₂₀ alkyl)bicyclo[2.2.1]heptyl group, a (C₁-C₂₀ alkyl)bicyclo[2.2.2]octyl group, a phenyl group, a (C₁-C₂₀ alkyl)phenyl group, a biphenyl group, a terphenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, an isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, an imidazopyrimidinyl group, an azacarbazolyl group, an azadibenzofuranyl group, an azadibenzothiophenyl group, —Si(Q₂₁)(Q₂₂)(Q₂₃), —Ge(Q₂₁)(Q₂₂)(Q₂₃), —C(Q₂₁)(Q₂₂)(Q₂₃), —B(Q₂₁)(Q₂₂), —N(Q₂₁)(Q₂₂) or any combination thereof, or

—Si(Q₁)(Q₂)(Q₃), —Ge(Q₁)(Q₂)(Q₃), —C(Q₁)(Q₂)(Q₃), —B(Q₁)(Q₂), or —N(Q₁)(Q₂),

wherein Q₁ to Q₃ and Q₂₁ to Q₂₃ may each independently be:

deuterium, —F, —CH₃, —CD₃, —CD₂H, —CDH₂, —CH₂CH₃, —CH₂CD₃, —CH₂CD₂H, —CH₂CDH₂, —CHDCH₃, —CHDCD₂H, —CHDCDH₂, —CHDCD₃, —CD₂CD₃, —CD₂CD₂H, —CD₂CDH₂, —CF₃, —CF₂H, —CFH₂, —CH₂CF₃, —CH₂CF₂H, —CH₂CFH₂, —CHFCH₃, —CHFCF₂H, —CHFCFH₂, —CHFCF₃, —CF₂CF₃, —CF₂CF₂H, or —CF₂CFH₂; or

an n-propyl group, an iso-propyl group, an n-butyl group, a sec-butyl group, an isobutyl group, a tert-butyl group, an n-pentyl group, a tert-pentyl group, a neopentyl group, an isopentyl group, a sec-pentyl group, a 3-pentyl group, a sec-isopentyl group, a phenyl group, a biphenyl group, or a naphthyl group, each unsubstituted or substituted with deuterium, —F, a C₁-C₁₀ alkyl group, a phenyl group, or any combination thereof.

In some embodiments, in Formulae 1-1 and 1-2, R₁₁ to R₁₉ may each independently be hydrogen, deuterium, —F, —CH₃, —CD₃, —CD₂H, —CDH₂, —CF₃, —CF₂H, —CFH₂, C₂-C₁₀ alkenyl group, a C₁-C₁₀ alkoxy group, a C₁-C₁₀alkylthio group, a group represented by one of Formulae 9-1 to 9-39, a group represented by one of Formulae 9-1 to 9-39 in which at least one hydrogen is substituted with deuterium, a group represented by one of Formulae 9-1 to 9-39 in which at least one hydrogen is substituted with —F, a group represented by one of Formulae 9-201 to 9-236, a group represented by one of Formulae 9-201 to 9-236 in which at least one hydrogen is substituted with deuterium, a group represented by one of Formulae 9-201 to 9-236 in which at least one hydrogen is substituted with —F, a group represented by one of Formulae 10-1 to 10-130, a group represented by one of Formulae 10-1 to 10-130 in which at least one hydrogen is substituted with deuterium, a group represented by one of Formulae 10-1 to 10-130 in which at least one hydrogen is substituted with —F, a group represented by one of Formulae 10-201 to 10-358, a group represented by one of Formulae 10-201 to 10-358 in which at least one hydrogen is substituted with deuterium, a group represented by one of Formulae 10-201 to 10-358 in which at least one hydrogen is substituted with —F, a group represented by one of Formulae 10-359 to 10-380, a group represented by one of Formulae 10-359 to 10-380 in which at least one hydrogen is substituted with deuterium, a group represented by one of Formulae 10-359 to 10-380 in which at least one hydrogen is substituted with —F, —Si(Q₁)(Q₂)(Q₃), —Ge(Q₁)(Q₂)(Q₃), —C(Q₁)(Q₂)(Q₃), —B(Q₁)(Q₂), or —N(Q₁)(Q₂),

wherein Q₁ to Q₃ may each independently be:

deuterium, —F, —CH₃, —CD₃, —CD₂H, —CDH₂, —CH₂CH₃, —CH₂CD₃, —CH₂CD₂H, —CH₂CDH₂, —CHDCH₃, —CHDCD₂H, —CHDCDH₂, —CHDCD₃, —CD₂CD₃, —CD₂CD₂H, —CD₂CDH₂, —CF₃, —CF₂H, —CFH₂, —CH₂CF₃, —CH₂CF₂H, —CH₂CFH₂, —CHFCH₃, —CHFCF₂H, —CHFCFH₂, —CHFCF₃, —CF₂CF₃, —CF₂CF₂H, or —CF₂CFH₂; or

an n-propyl group, an iso-propyl group, an n-butyl group, a sec-butyl group, an isobutyl group, a tert-butyl group, an n-pentyl group, a tert-pentyl group, a neopentyl group, an isopentyl group, a sec-pentyl group, a 3-pentyl group, a sec-isopentyl group, a phenyl group, a biphenyl group, or a naphthyl group, each unsubstituted or substituted with deuterium, —F, a C₁-C₁₀ alkyl group, a phenyl group, or any combination thereof.

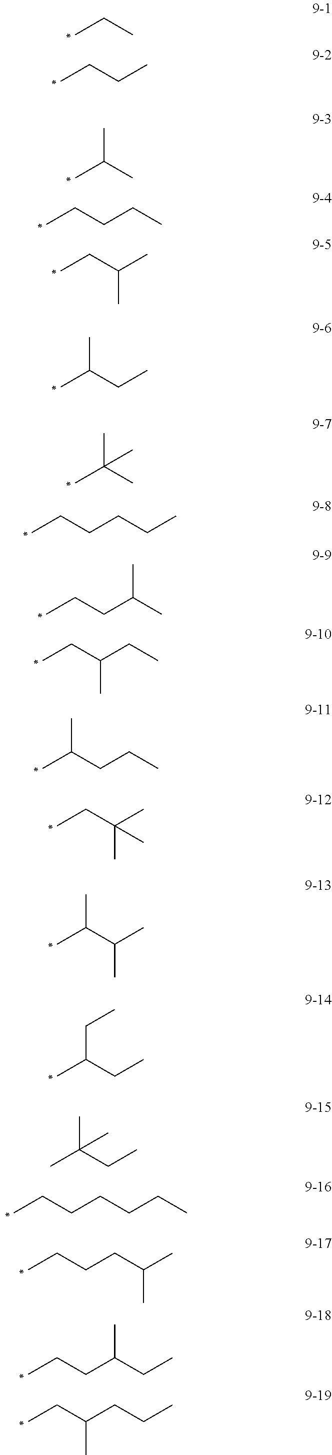

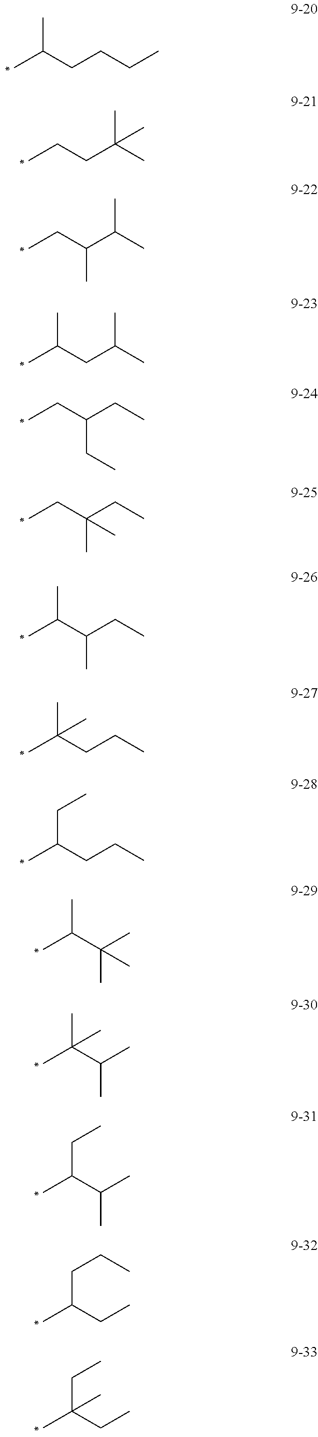

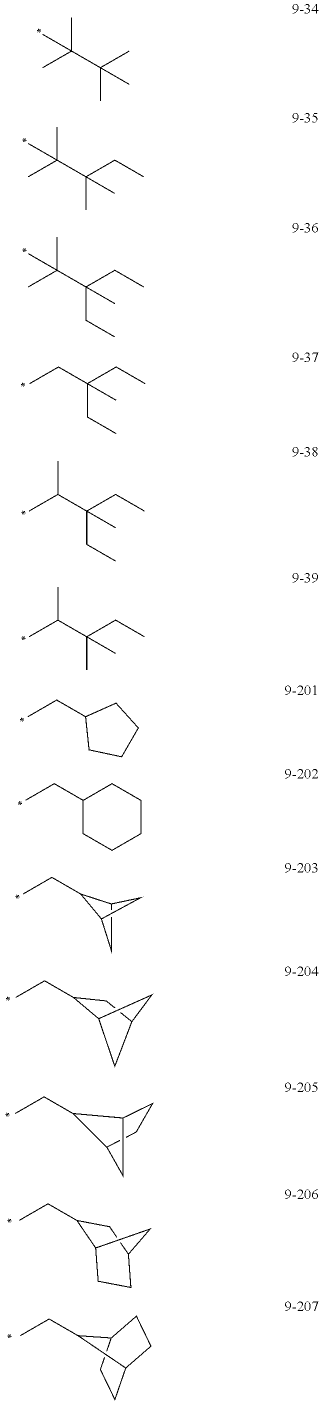

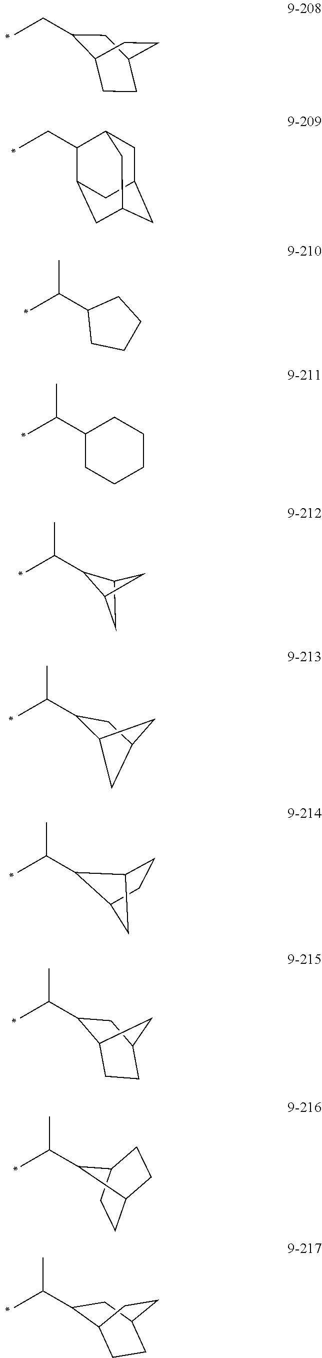

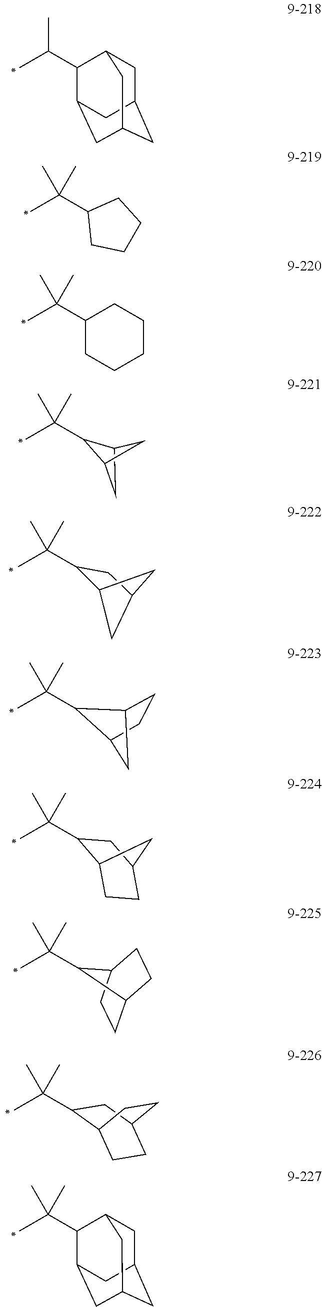

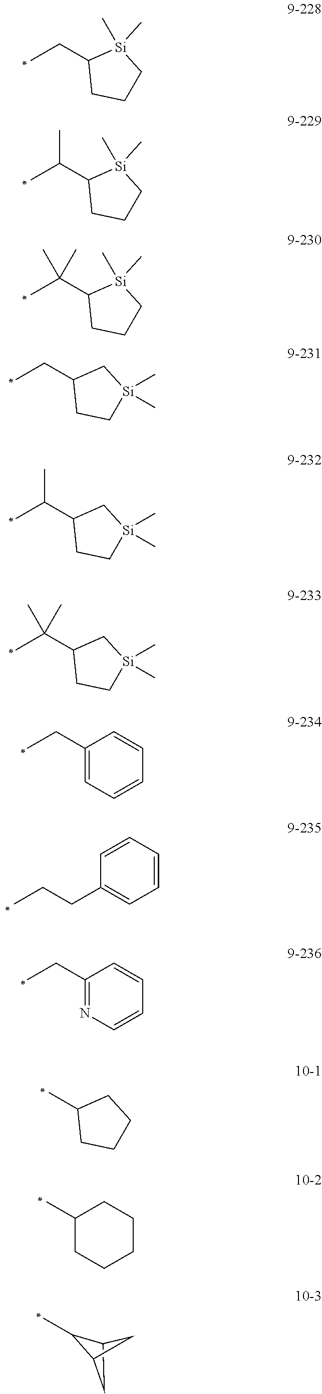

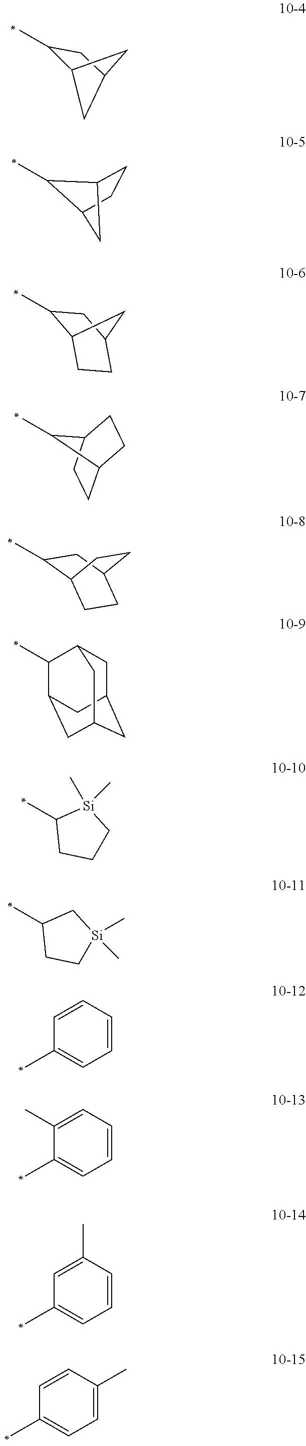

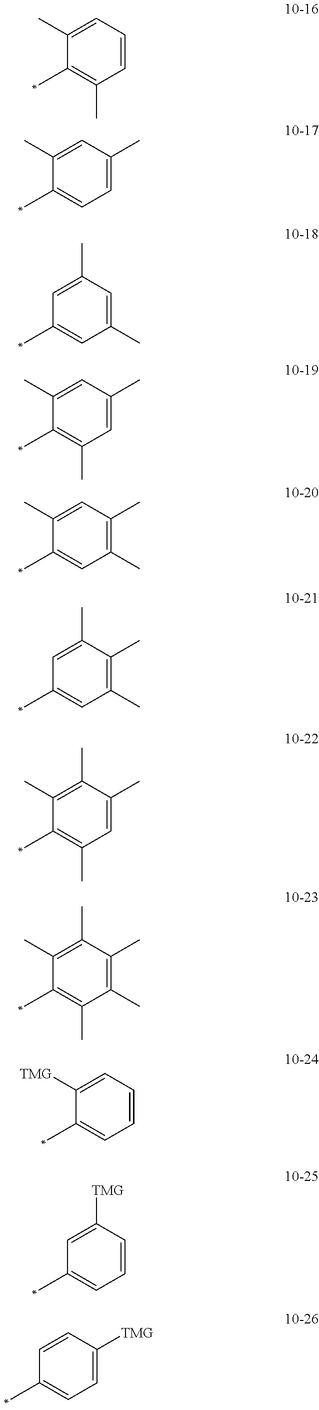

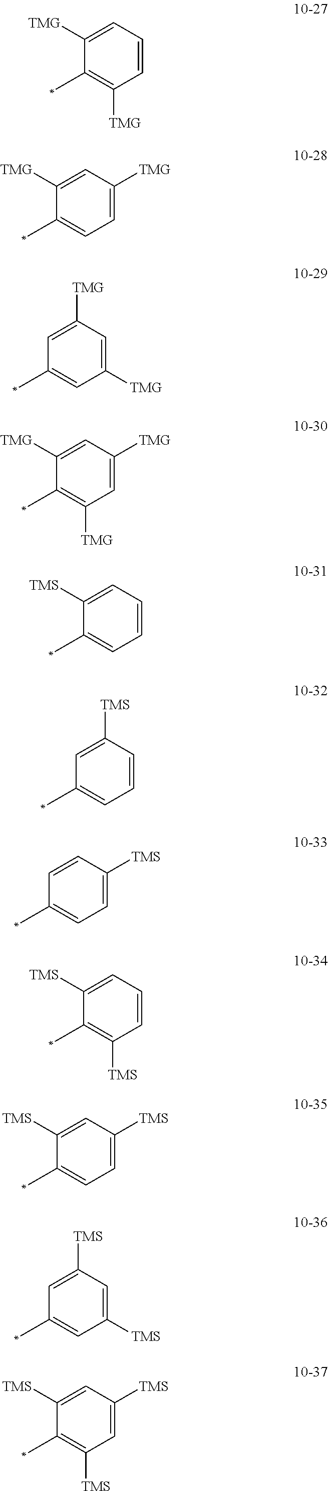

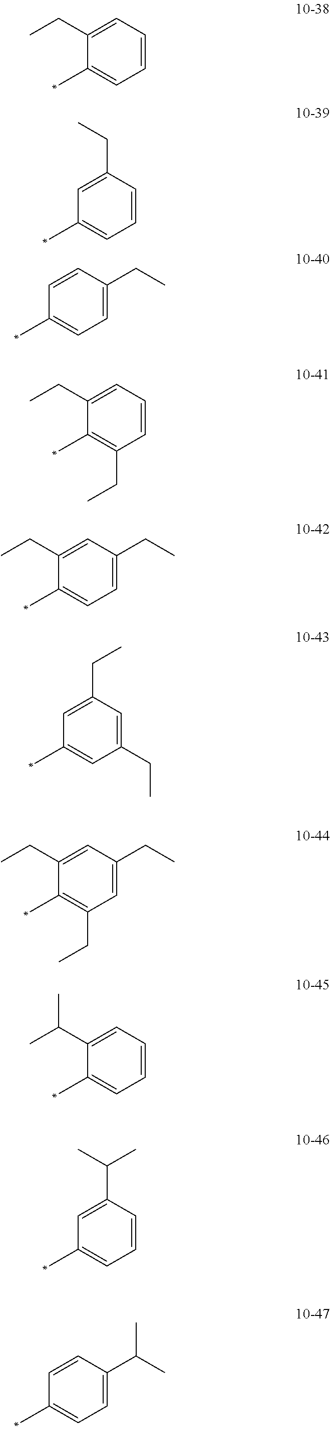

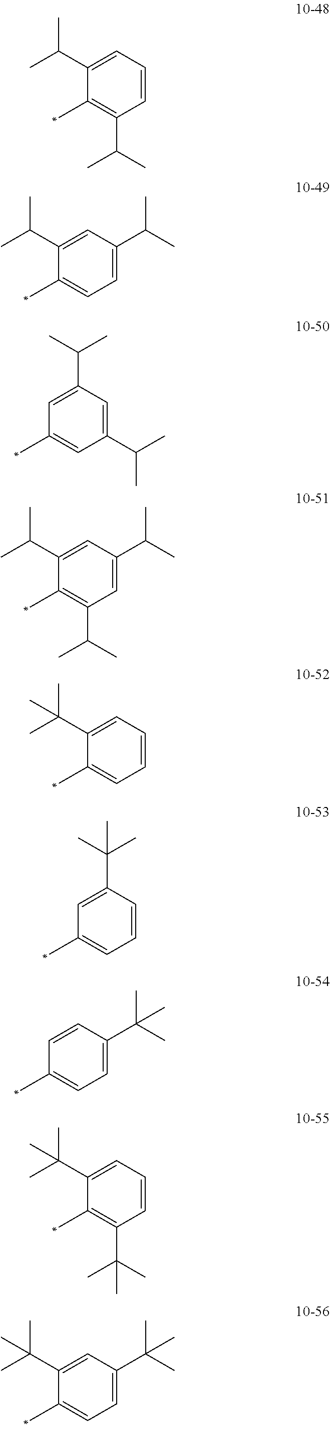

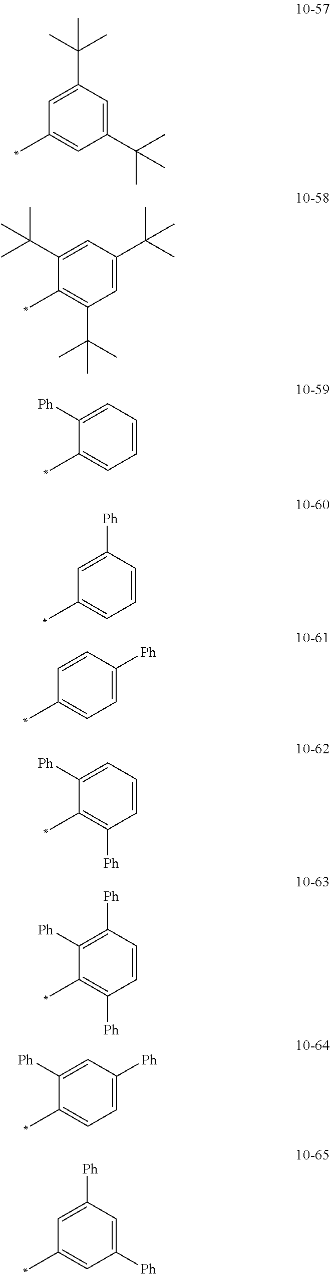

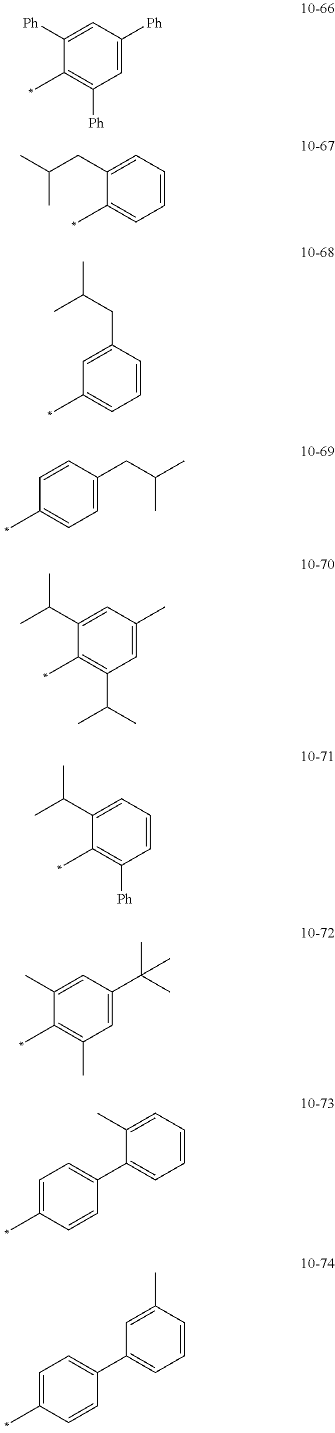

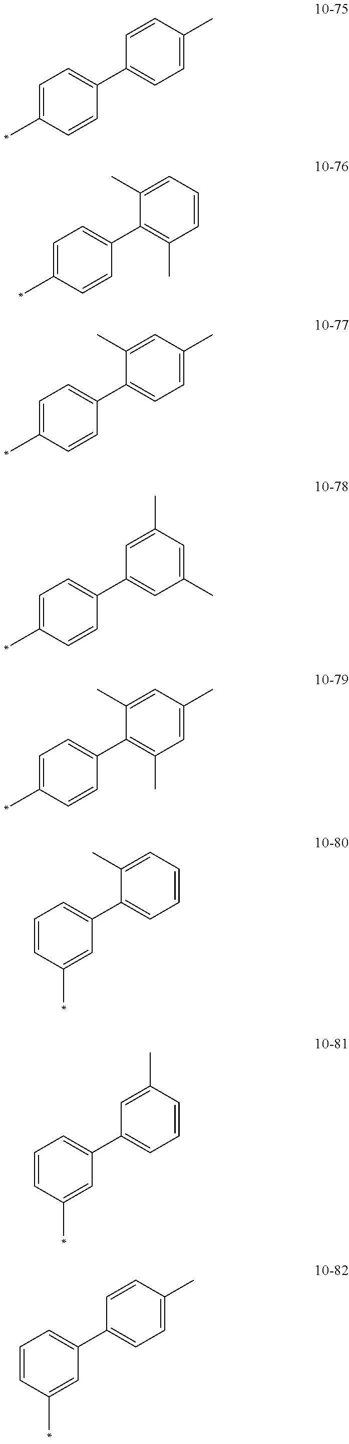

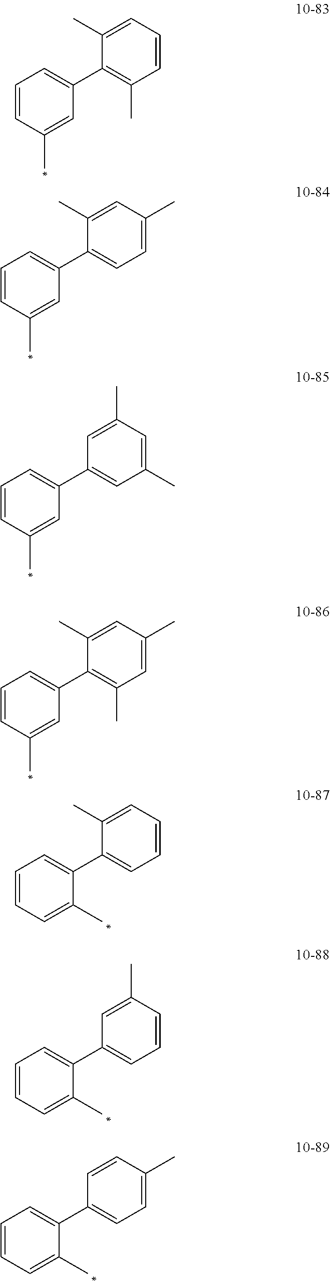

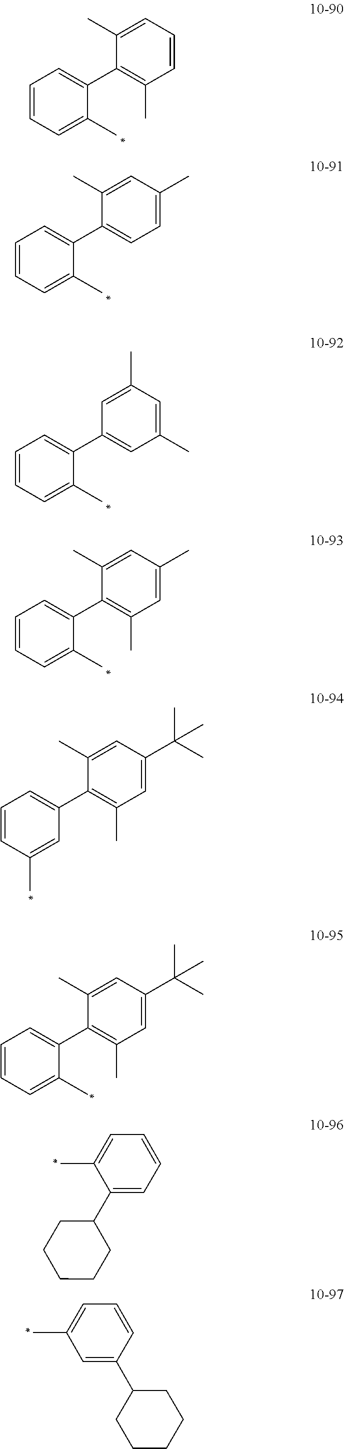

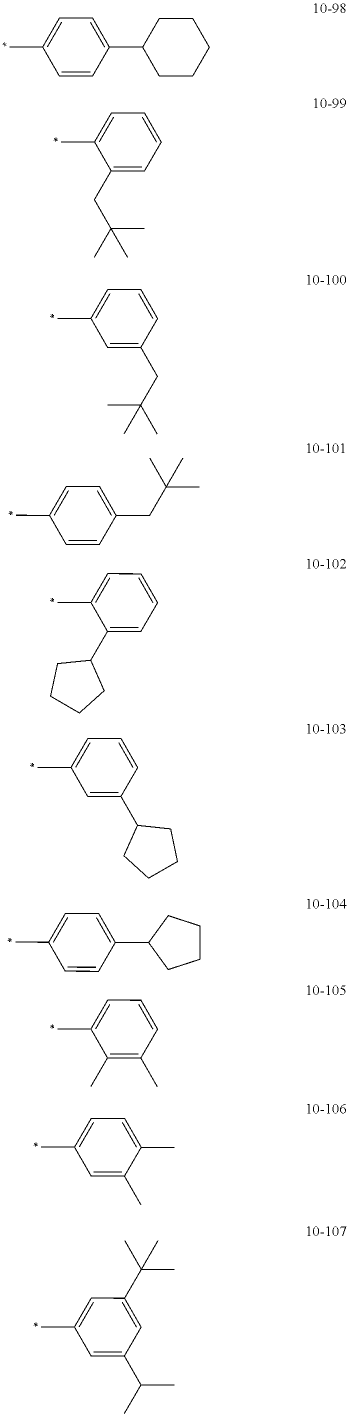

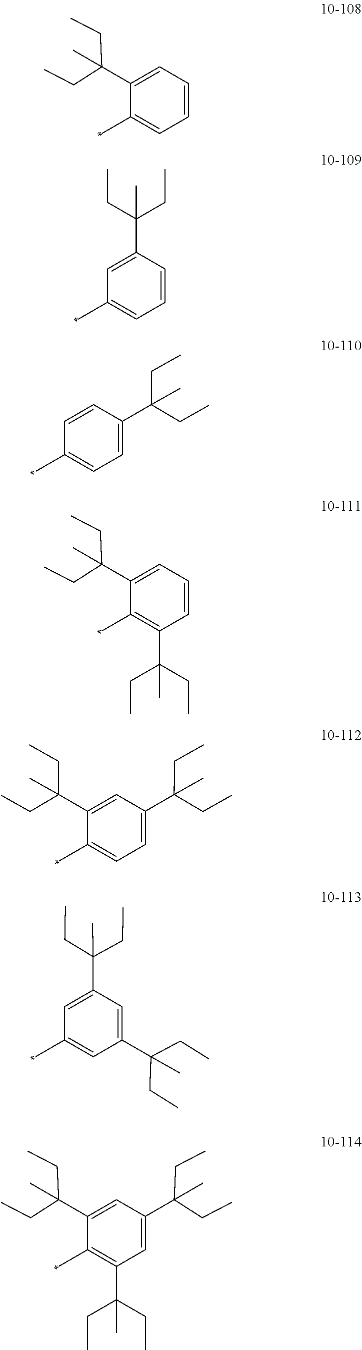

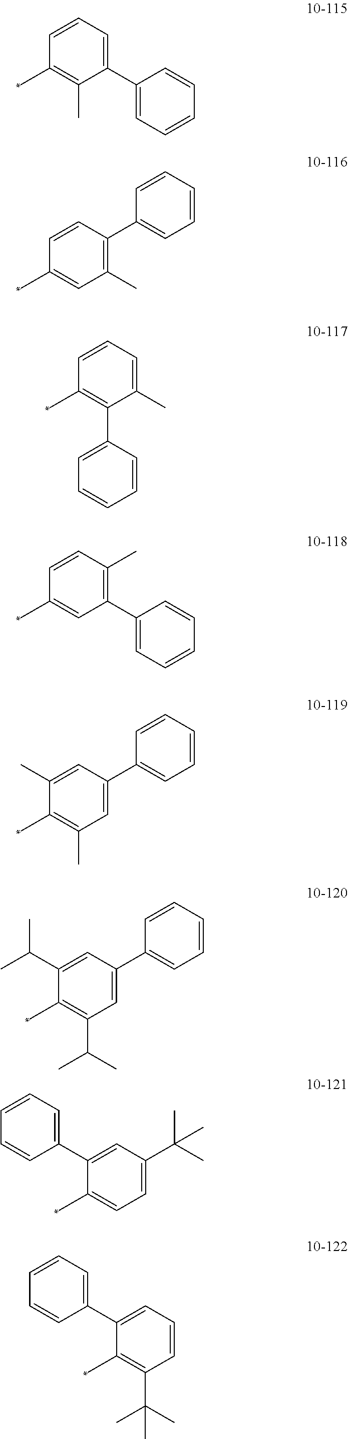

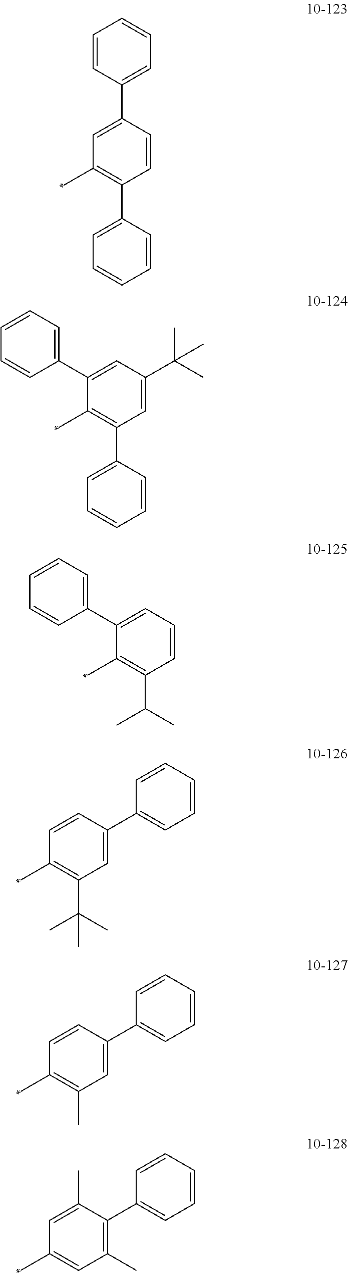

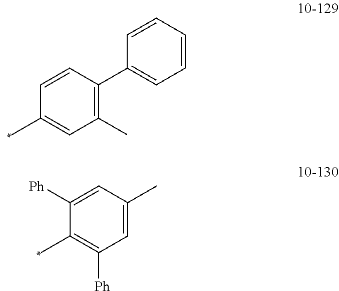

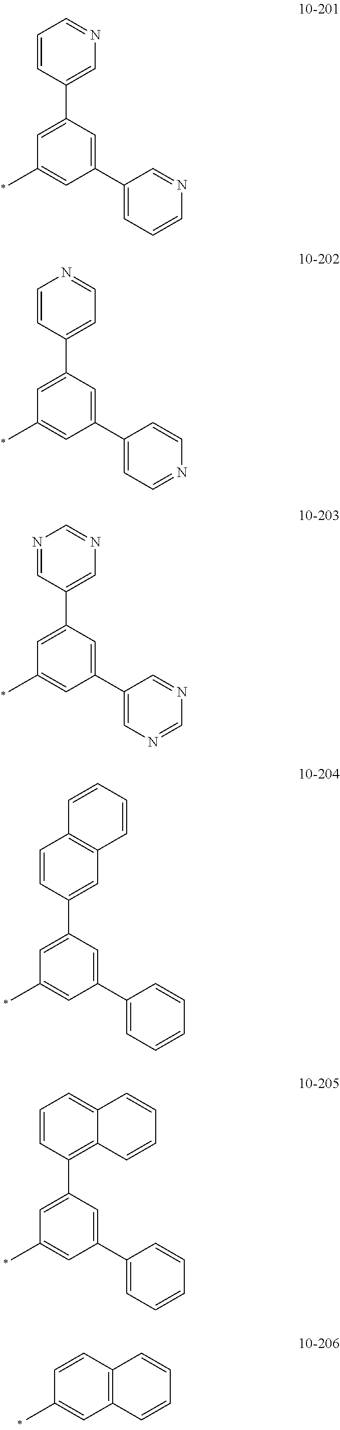

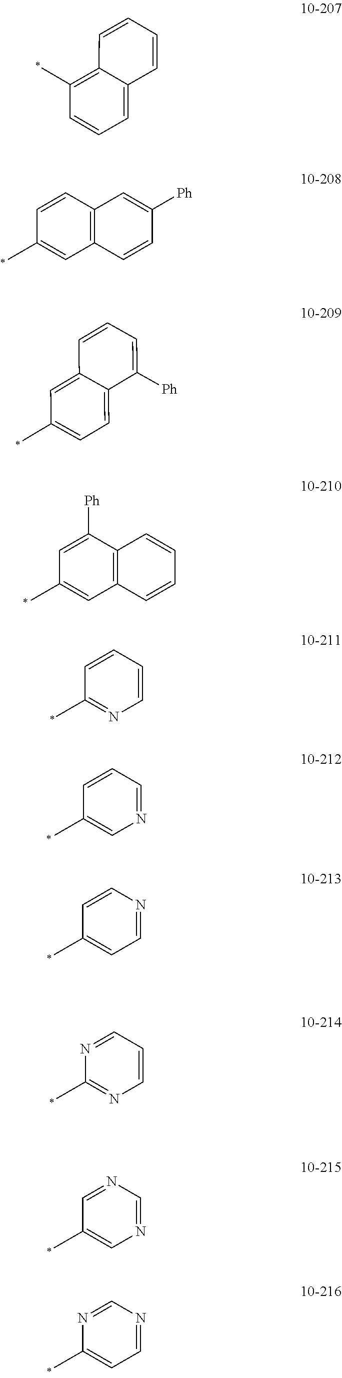

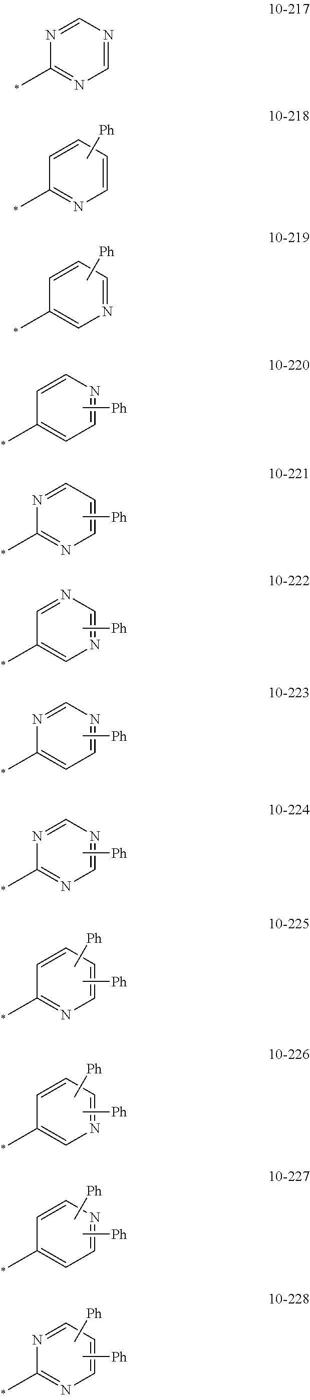

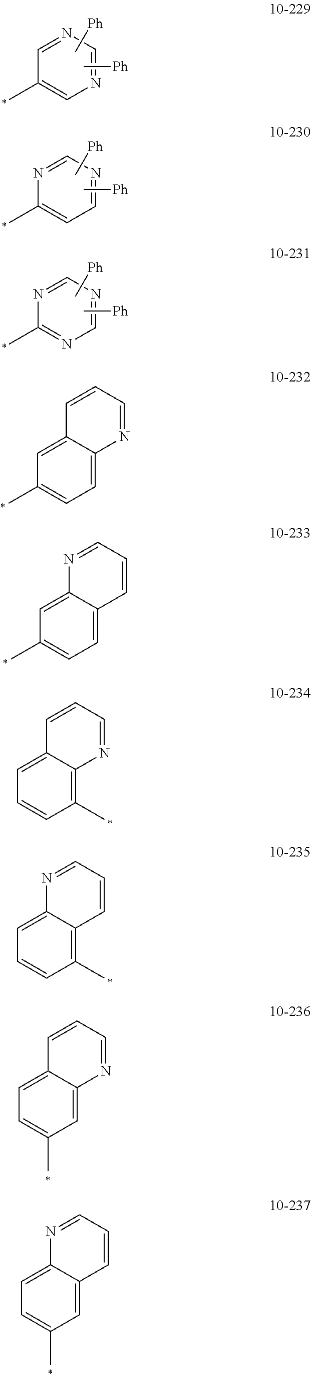

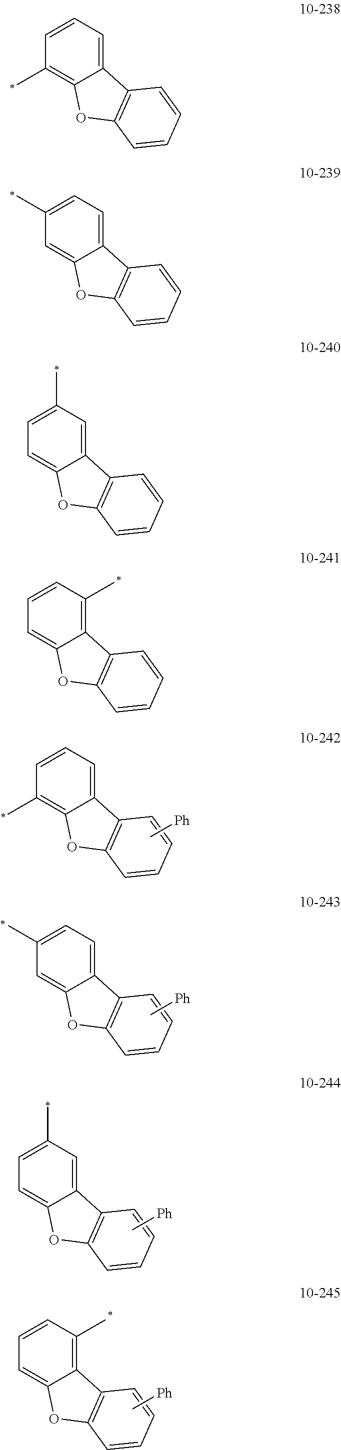

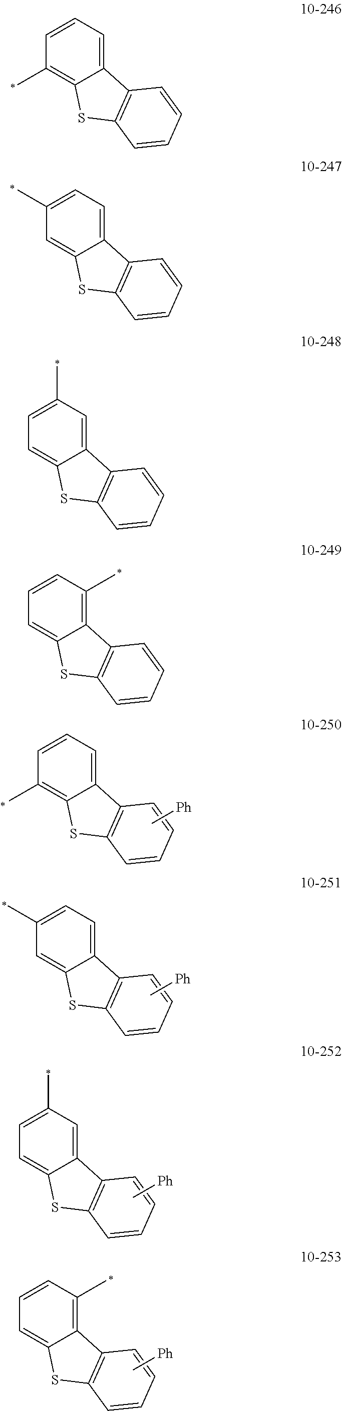

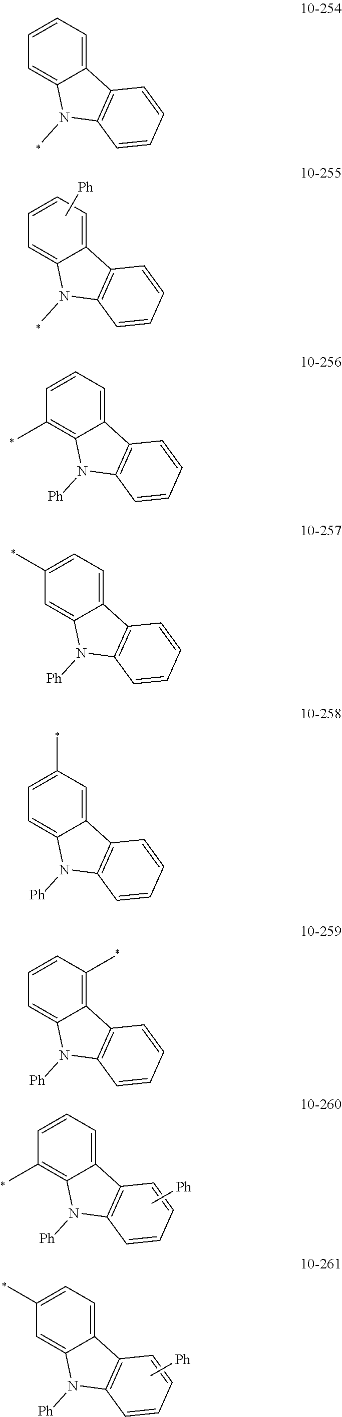

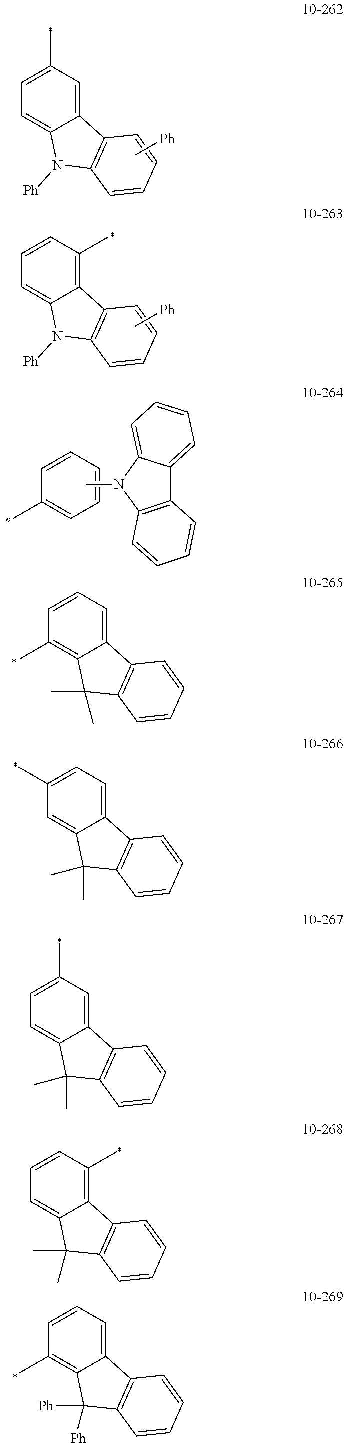

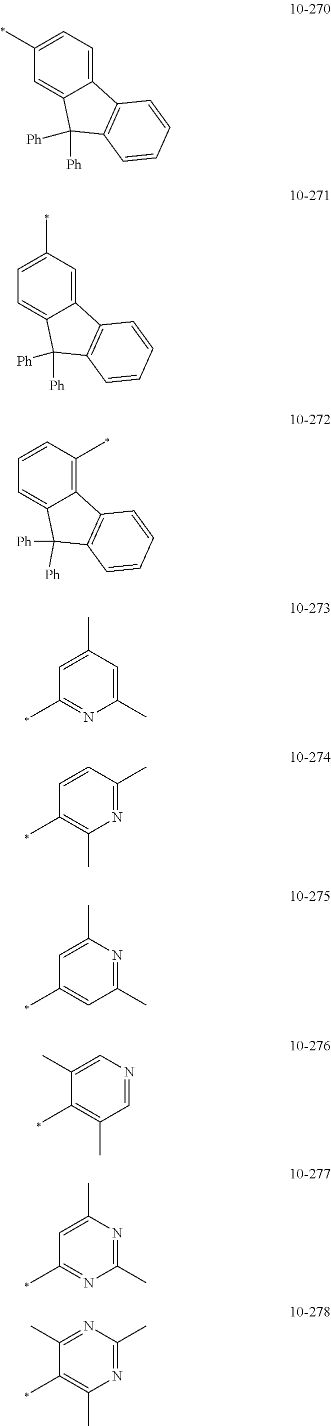

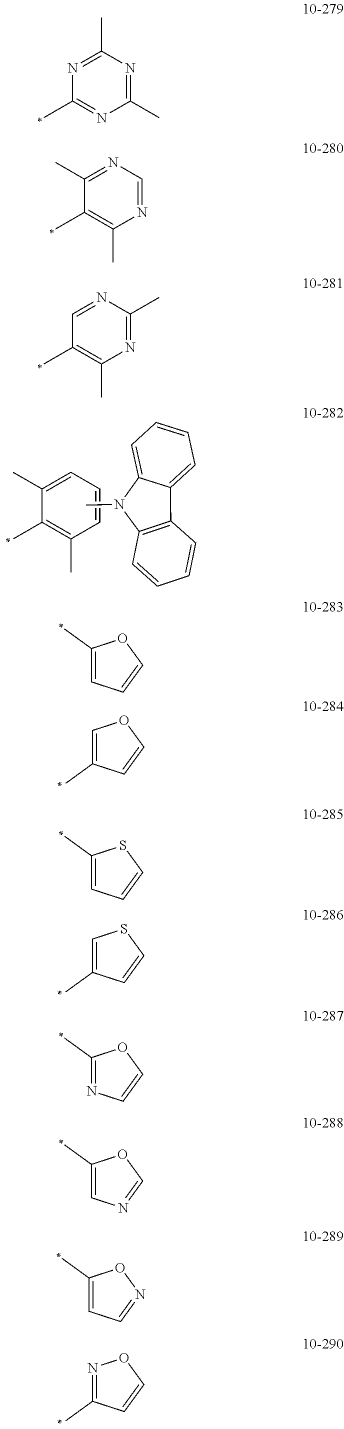

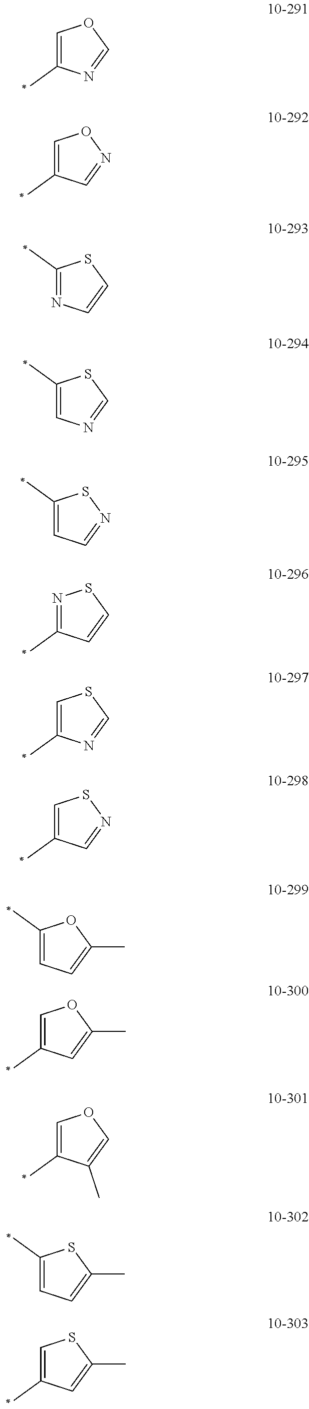

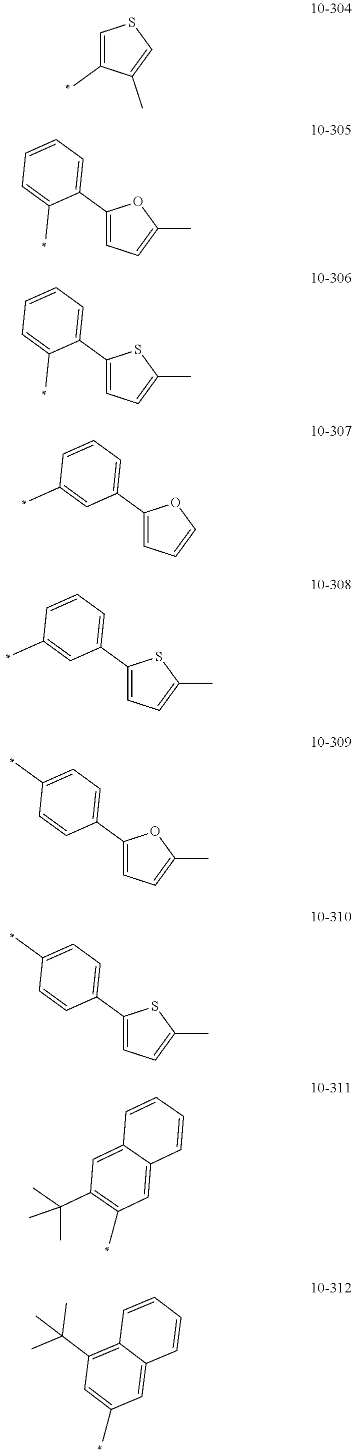

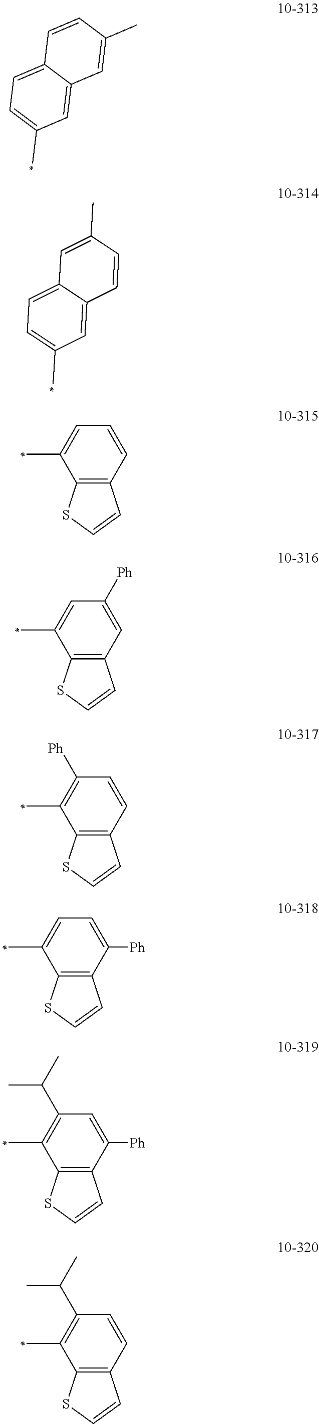

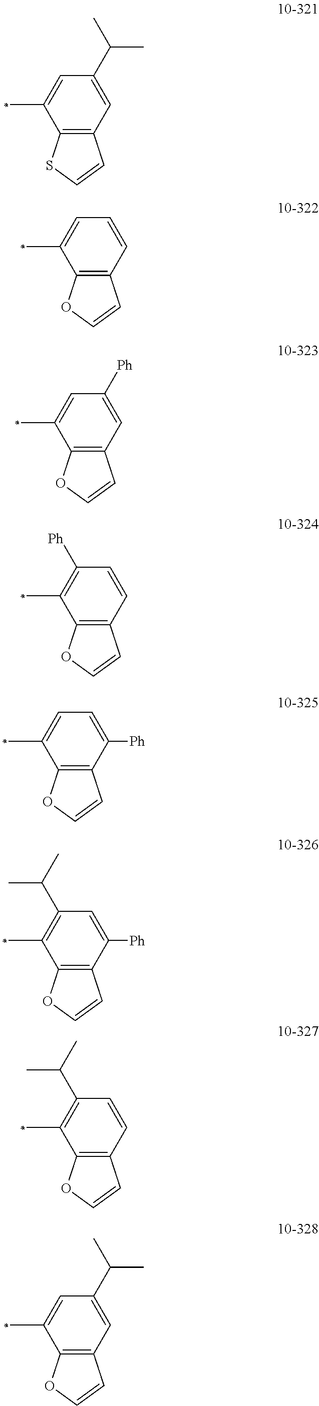

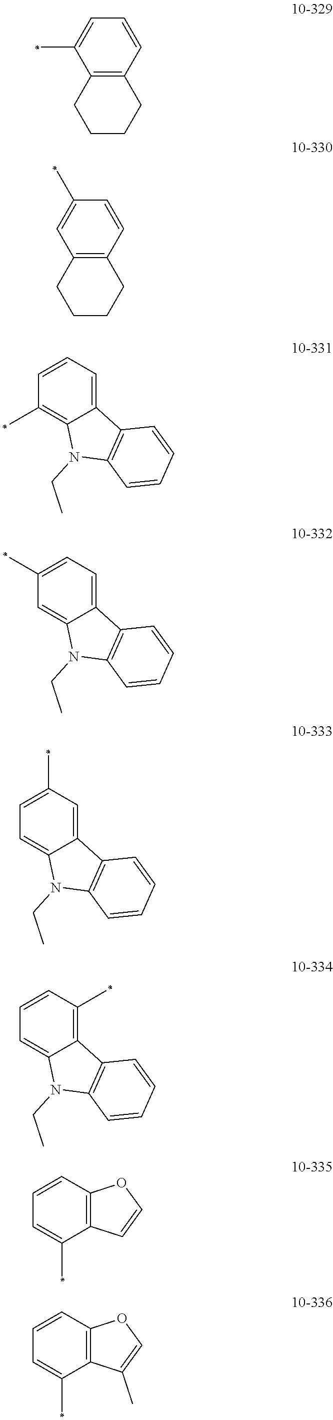

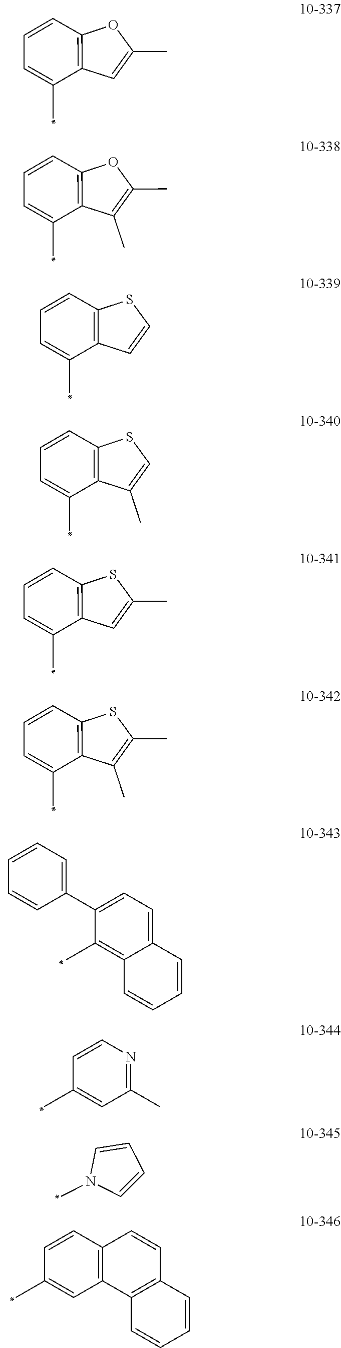

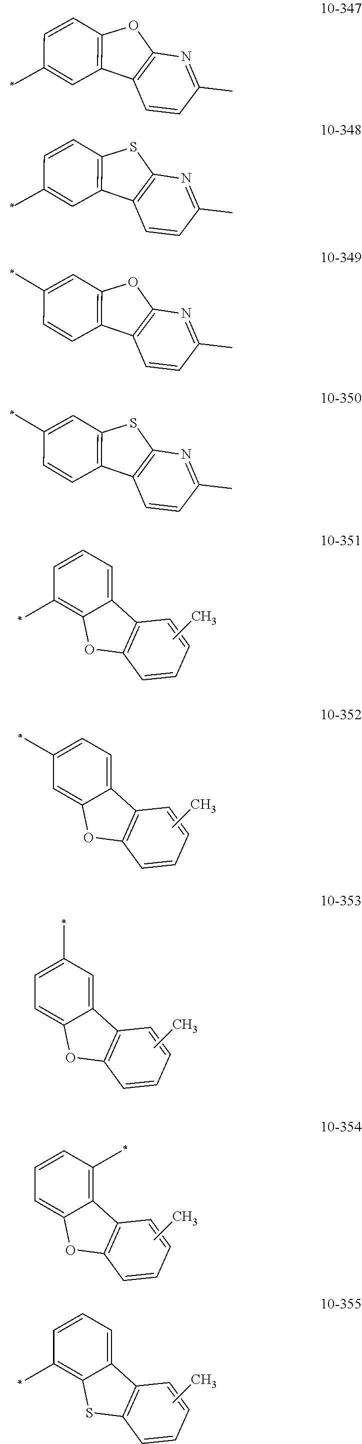

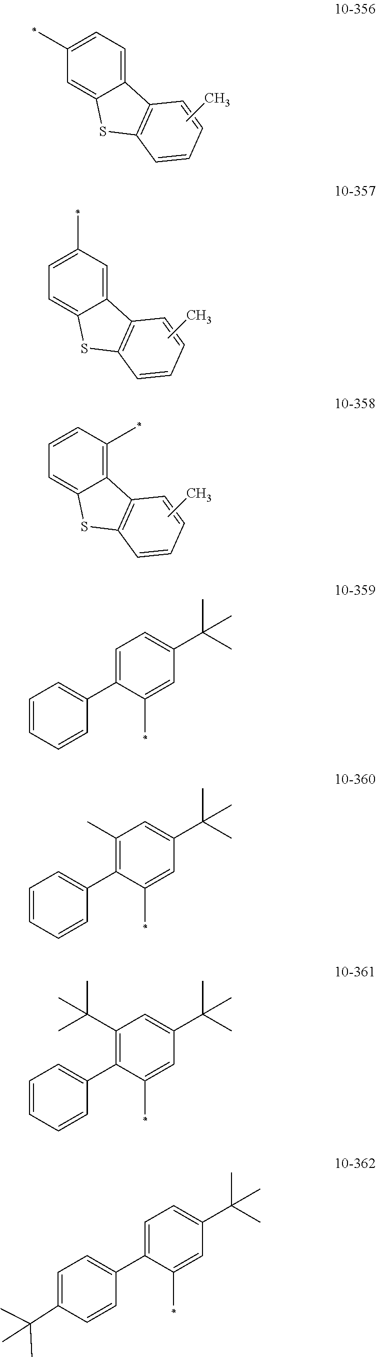

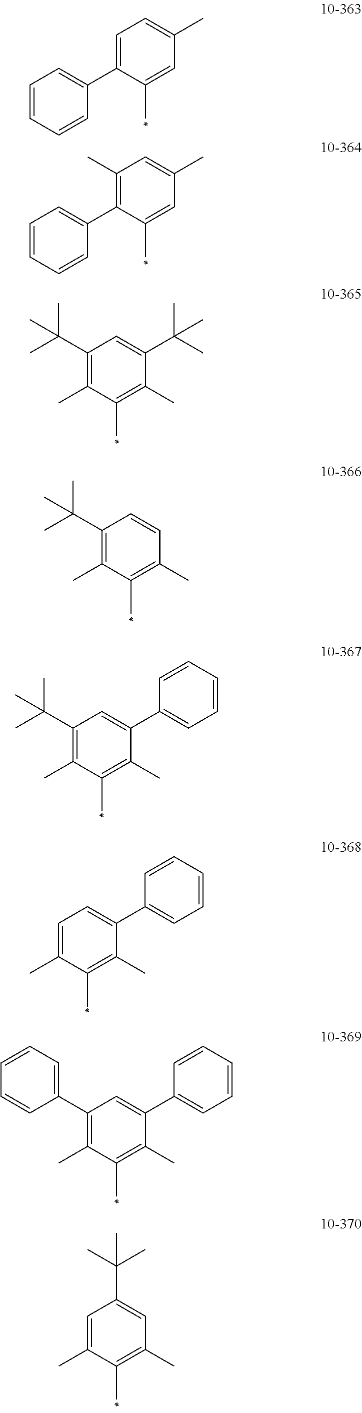

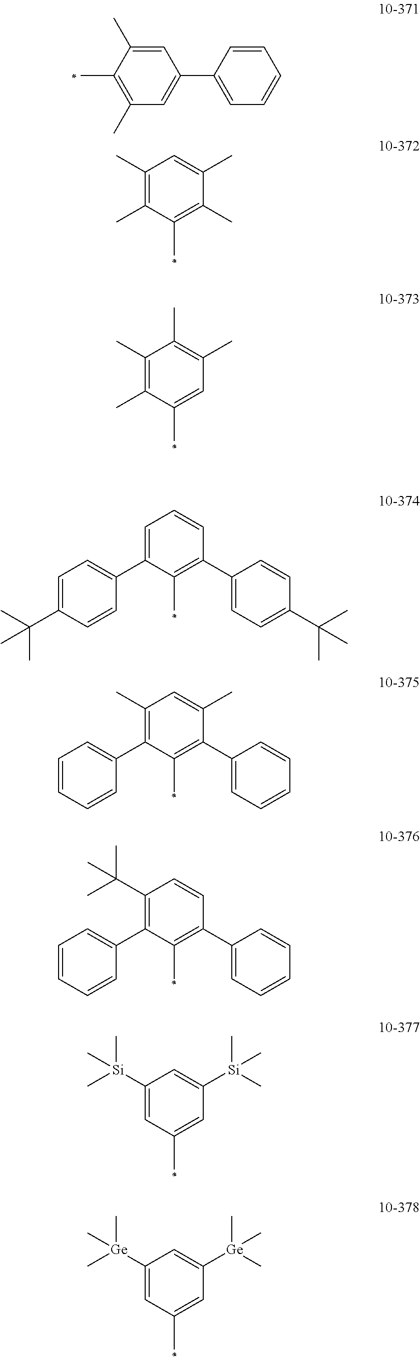

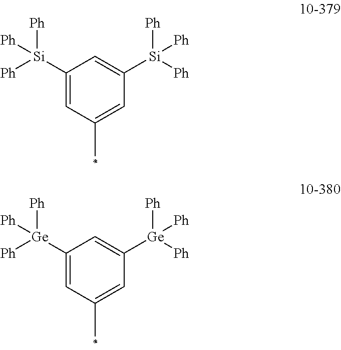

In Formulae 9-1 to 9-39, 9-201 to 9-236, 10-1 to 10-130, and 10-201 to 10-380, * indicates a binding site to an adjacent atom, “Ph” represents a phenyl group, “TMS” and “SiMe₃” each represent a trimethylsilyl group, and “TMG” and “GeMe₃” each represent a trimethylgermyl group.

The “group represented by Formulae 9-1 to 9-39 in which at least one hydrogen is substituted with deuterium” and the “group represented by Formulae 9-201 to 9-236 in which at least one hydrogen is substituted with deuterium” may each be, for example, a group represented by one of Formulae 9-501 to 9-514 and 9-601 to 9-636:

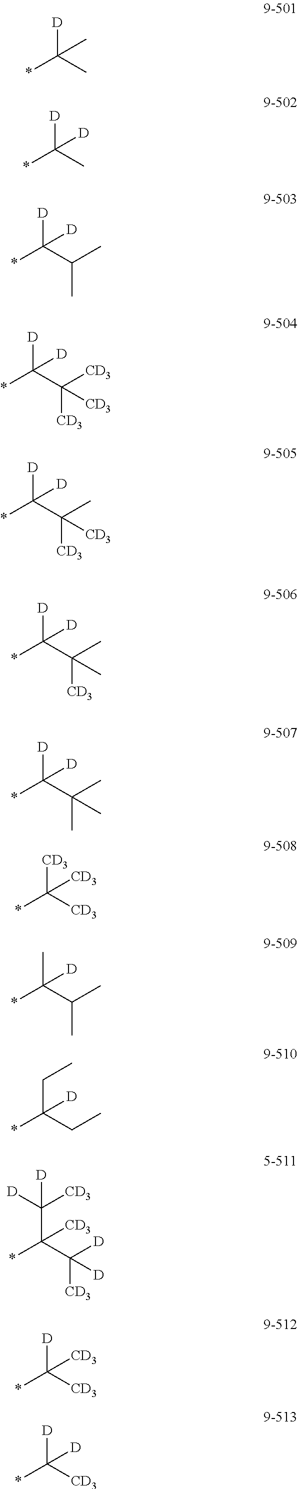

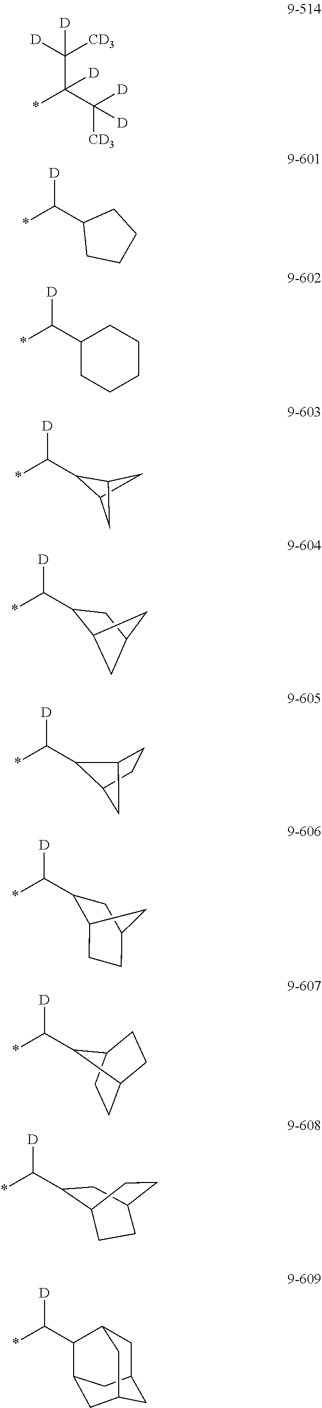

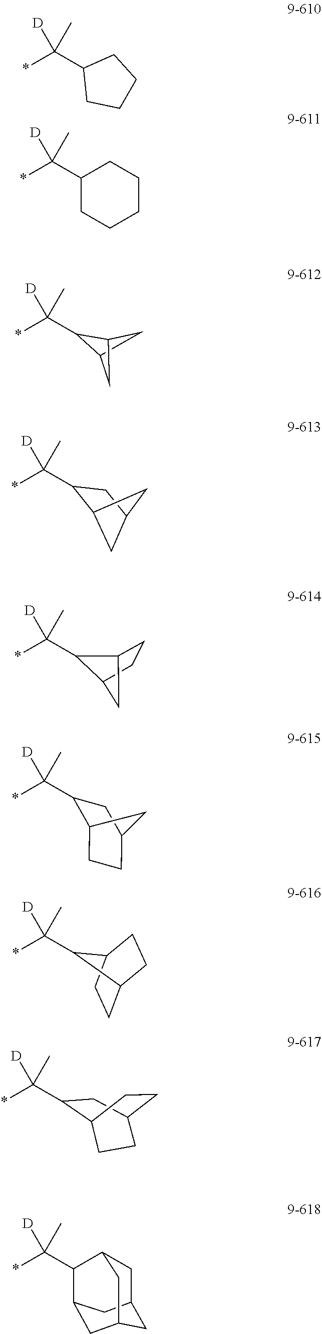

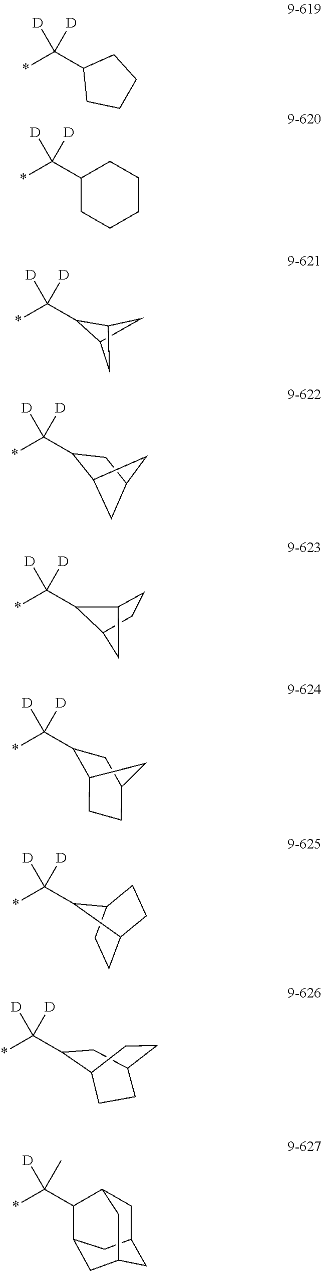

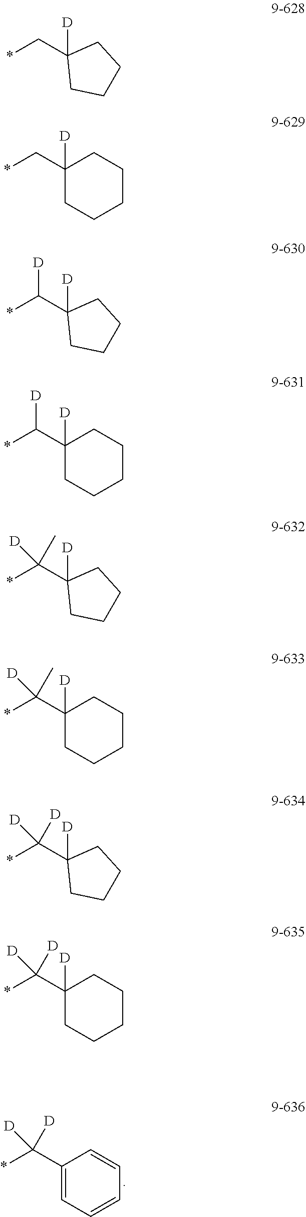

The “group represented by Formulae 9-1 to 9-39 in which at least one hydrogen is substituted with —F″ and the “group represented by Formulae 9-201 to 9-236 in which at least one hydrogen is substituted with —F″ may each be, for example, a group represented by one of Formulae 9-701 to 710:

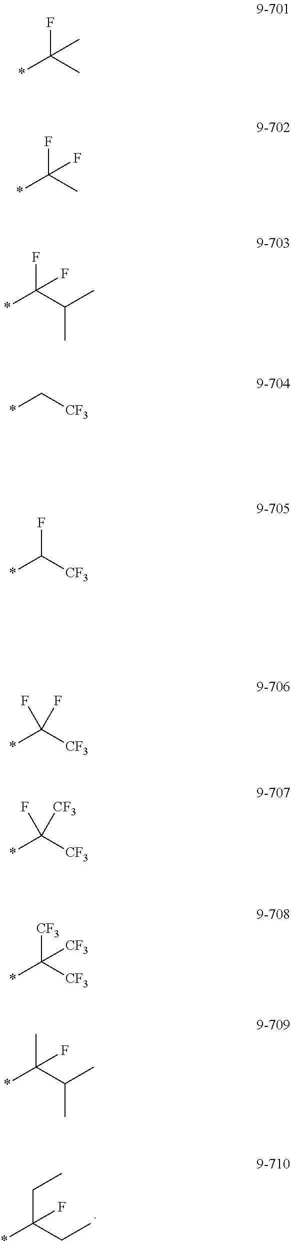

The “group represented by Formulae 10-1 to 10-130 in which at least one hydrogen is substituted with a deuterium” and the “group represented by Formulae 10-201 to 10-358 in which at least one hydrogen is substituted with deuterium” may each be, for example, a group represented by one of Formulae 10-501 to 576:

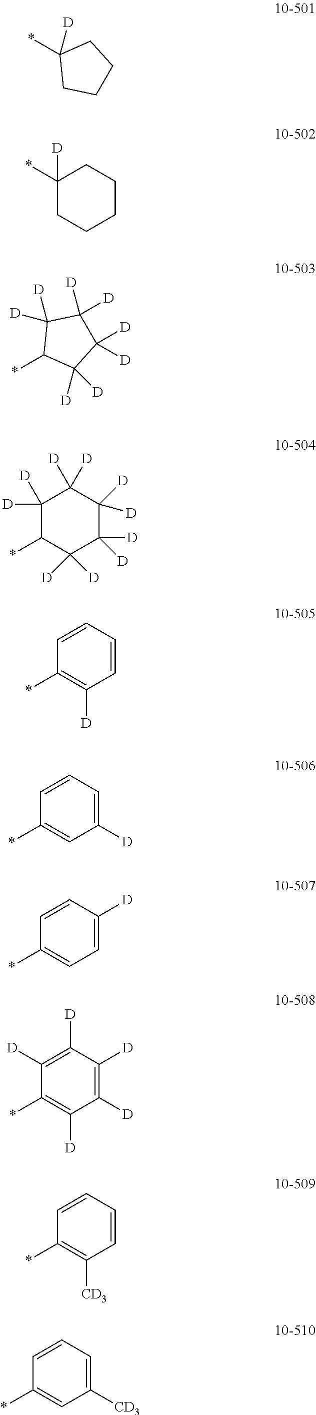

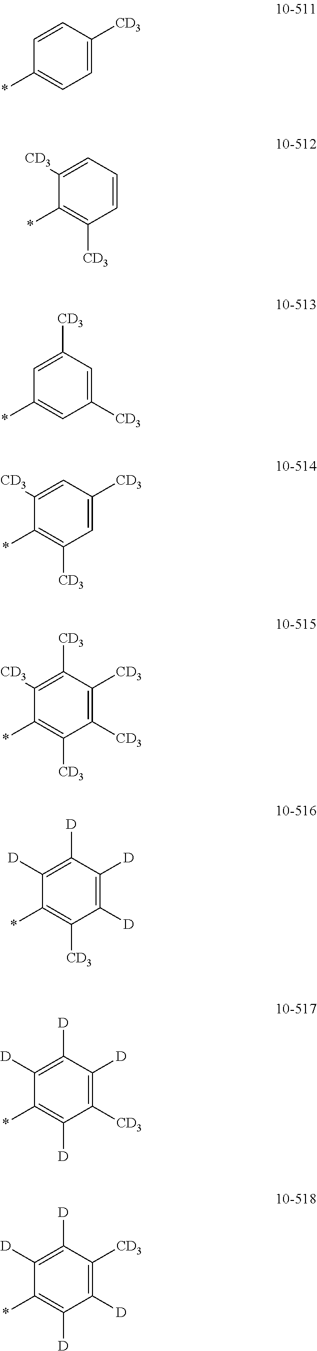

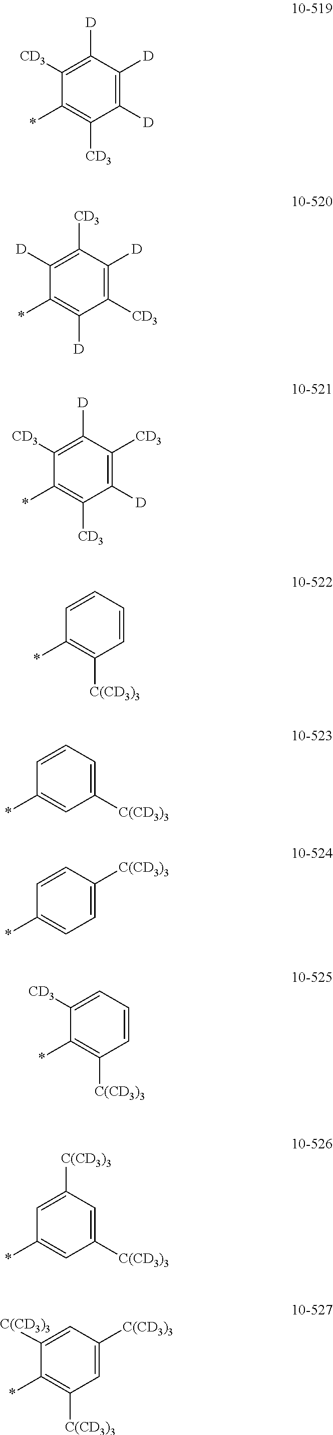

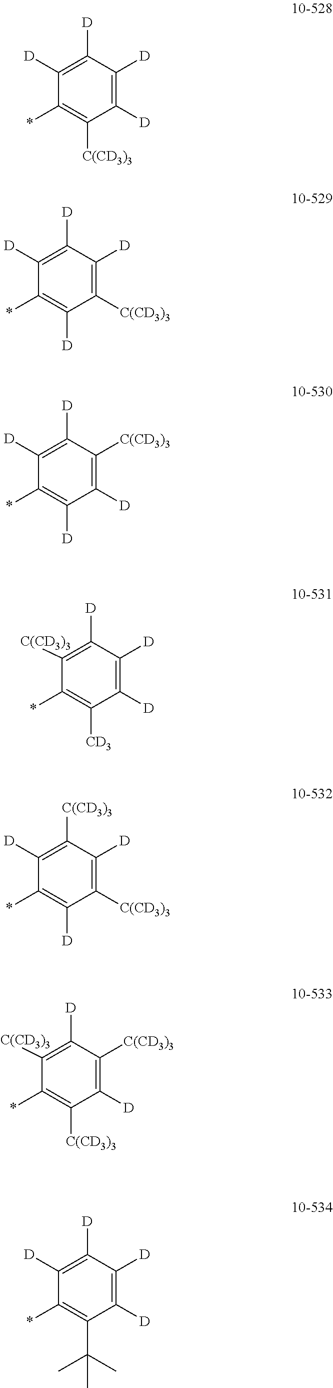

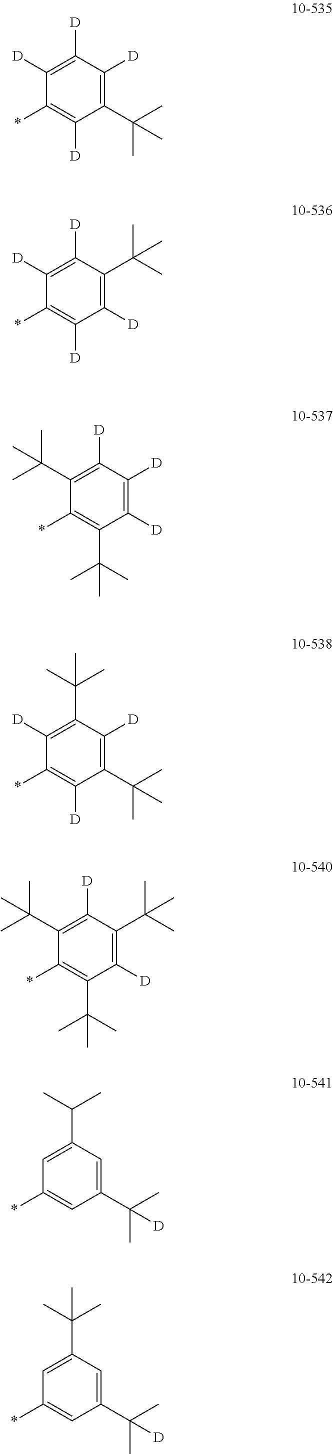

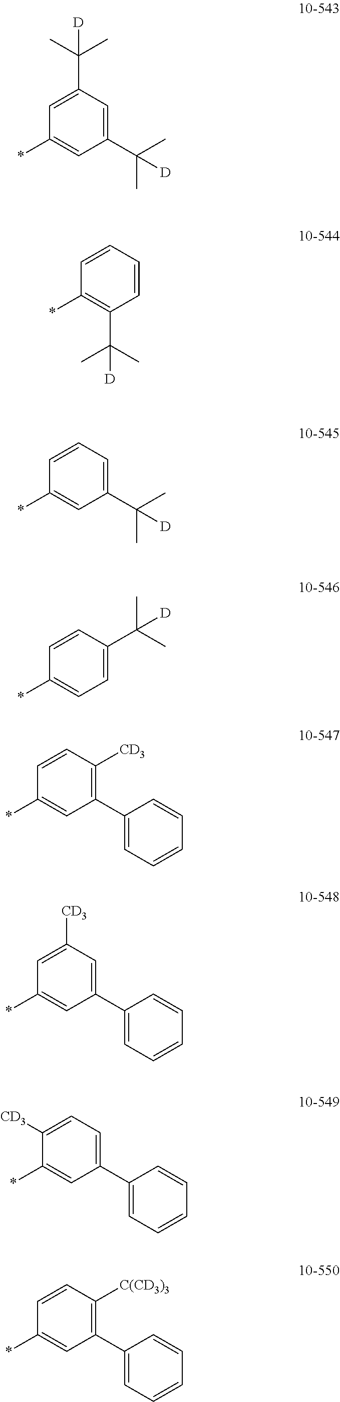

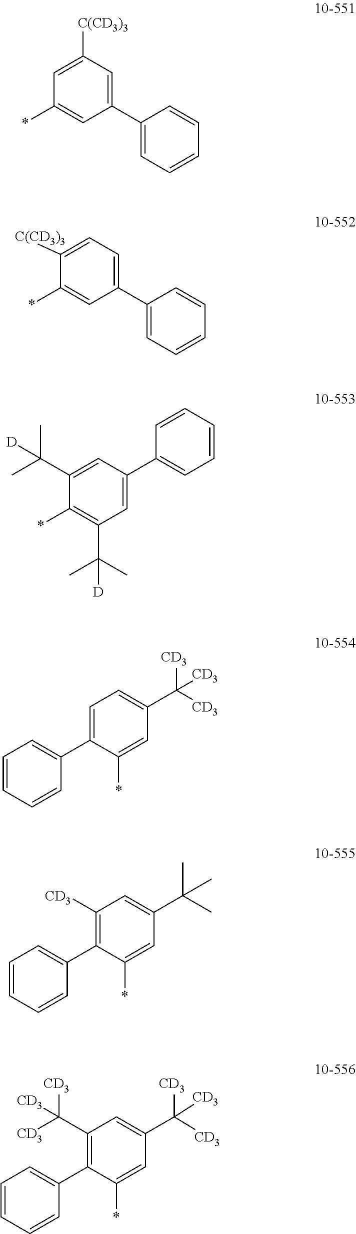

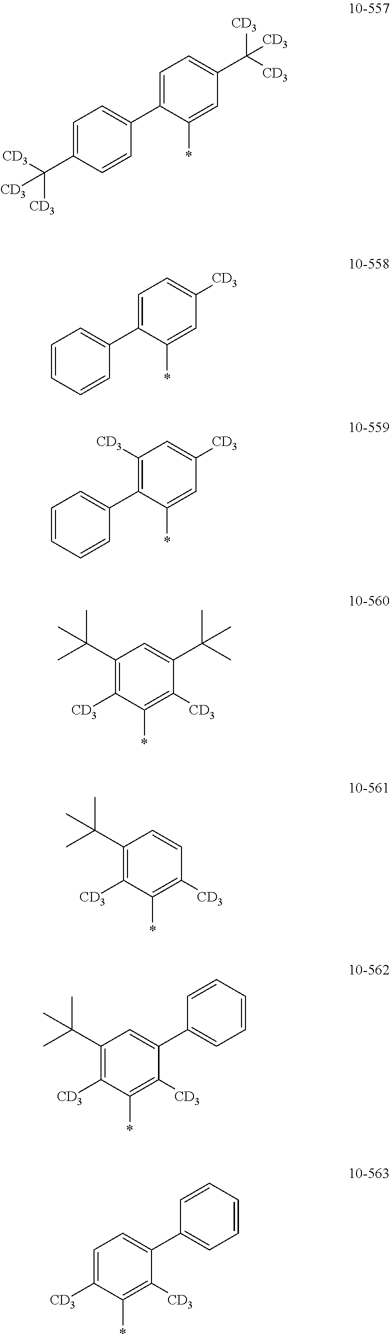

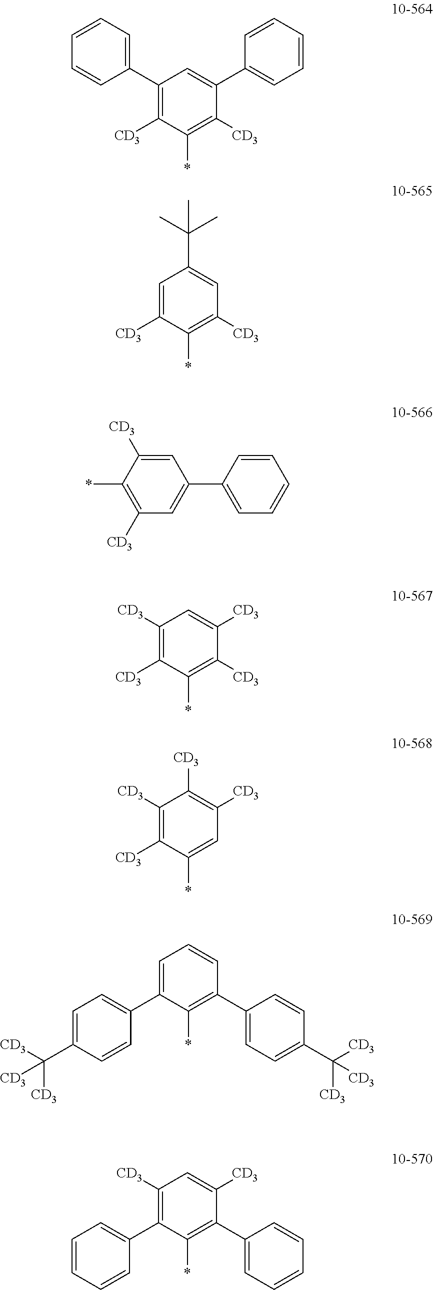

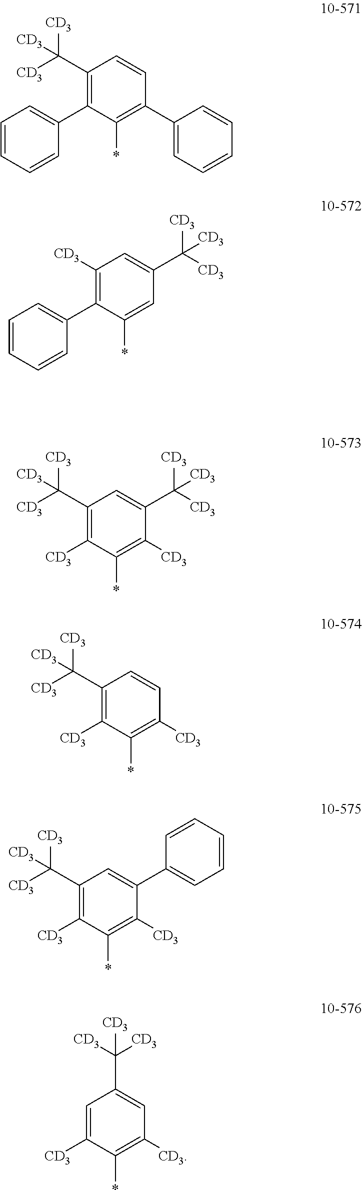

The “group represented by Formulae 10-1 to 10-130 in which at least one hydrogen is substituted with —F″ and the “group represented by Formulae 10-201 to 10-358 in which at least one hydrogen is substituted with —F″ may each be, for example, a group represented by one of Formulae 10-601 to 10-617:

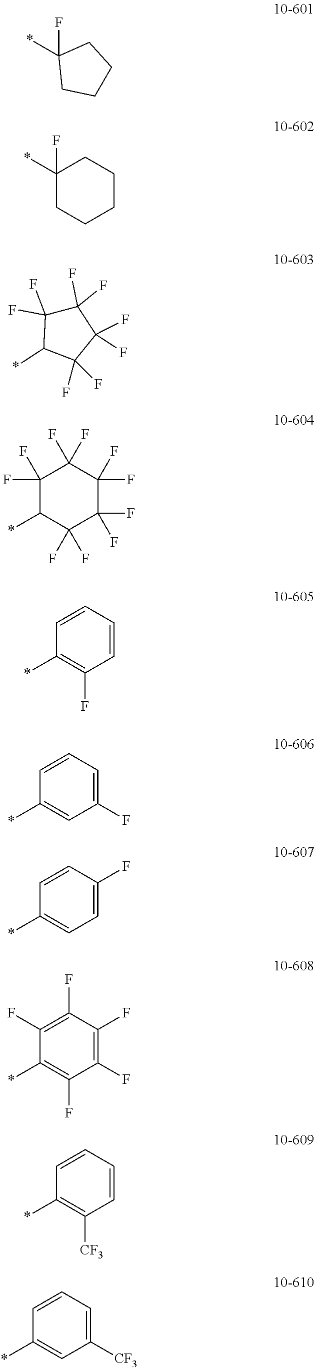

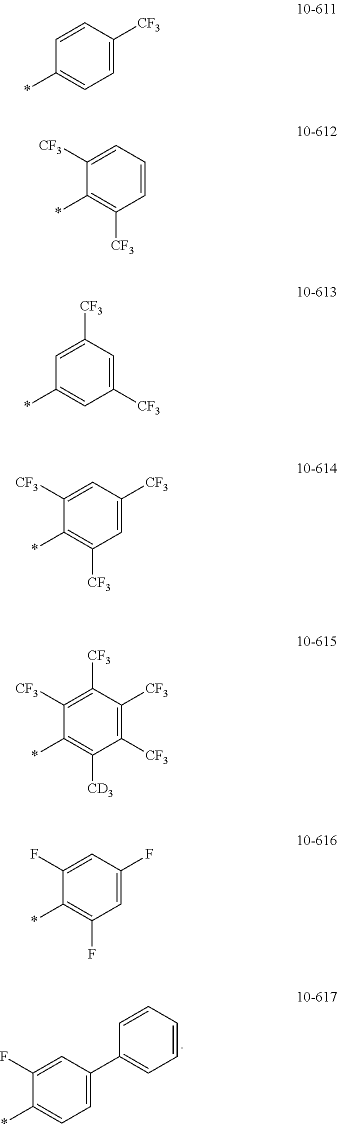

In some embodiments, in Formulae 1-1 and 1-2, R₁₁ to R₁₉ may each independently be hydrogen, deuterium, —F, —CH₃, —CD₃, —CD₂H, —CDH₂, —CF₃, —CF₂H, —CFH₂, a group represented by one of Formulae 9-1 to 9-39, a group represented by one of Formulae 9-1 to 9-39 in which at least one hydrogen is substituted with deuterium, a group represented by one of Formulae 9-1 to 9-39 in which at least one hydrogen is substituted with —F, a group represented by one of Formulae 10-1 to 10-130, a group represented by one of Formulae 10-1 to 10-130 in which at least one hydrogen is substituted with deuterium, a group represented by one of Formulae 10-1 to 10-130 in which at least one hydrogen is substituted with —F, a group represented by one of Formulae 10-359 to 10-380, a group represented by one of Formulae 10-359 to 10-380 in which at least one hydrogen is substituted with deuterium, a group represented by one of Formulae 10-359 to 10-380 in which at least one hydrogen is substituted with —F, —Si(Q₁)(Q₂)(Q₃), —Ge(Q₁)(Q₂)(Q₃), —C(Q₁)(Q₂)(Q₃), —B(Q₁)(Q₂), or —N(Q₁)(Q₂), wherein Q₁ to Q₃ may each independently be: deuterium, —F, —CH₃, —CD₃, —CD₂H, —CDH₂, —CH₂CH₃, —CH₂CD₃, —CH₂CD₂H, —CH₂CDH₂, —CHDCH₃, —CHDCD₂H, —CHDCDH₂, —CHDCD₃, —CD₂CD₃, —CD₂CD₂H, —CD₂CDH₂, —CF₃, —CF₂H, —CFH₂, —CH₂CF₃, —CH₂CF₂H, —CH₂CFH₂, —CHFCH₃, —CHFCF₂H, —CHFCFH₂, —CHFCF₃, —CF₂CF₃, —CF₂CF₂H, or —CF₂CFH₂; or an n-propyl group, an iso-propyl group, an n-butyl group, a sec-butyl group, an isobutyl group, a tert-butyl group, an n-pentyl group, a tert-pentyl group, a neopentyl group, an isopentyl group, a sec-pentyl group, a 3-pentyl group, a sec-isopentyl group, a phenyl group, a biphenyl group, or a naphthyl group, each unsubstituted or substituted with deuterium, —F, a C₁-C₁₀ alkyl group, a phenyl group, or any combination thereof.

In Formulae 1-1 and 1-2, b11 to b19 may respectively indicate the number of substitution(s) of R₁₁ to R₁₉, and b11, b14, and b19 may each independently be 0, 1, 2, or 3, b12, b15, and b18 may each independently be 0, 1, 2, 3, or 4, and b13 and b16 may each independently be 0, 1, or 2, and b17 may be 0 or 1. In Formulae 1-1 and 1-2, when b11 to b19 are each at least two, at least two R₁₁(s) to R₁₉(s) may be identical to or different from each other.

In the heterocyclic compound, in Formula 1-1, a case where X₁₄ may be *1, X₁₅ may be *2, W₁₂ may be *1, W₁₁ may be *2, Y₁₅ may be *3, Y₁₄ may be *4, W₁₆ may be *3, and W₁₅ may be *4 may be excluded.

In some embodiments, a heterocyclic compound may not be represented by Formula X:

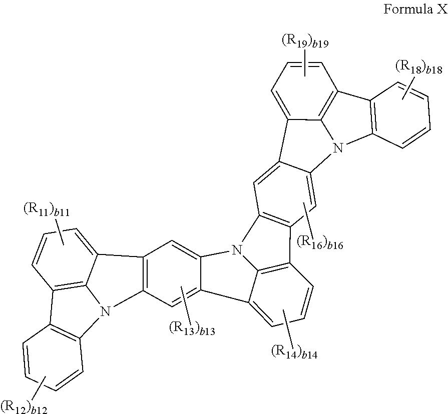

wherein, in Formula X, R₁₁ to R₁₄, R₁₆, R₁₈, R₁₉, b11 to b14, b16, b18, and b19 may respectively be understood by referring to the descriptions of R₁₁ to R₁₄, R₁₆, R₁₈, R₁₉, b11 to b14, b16, b18, and b19 provided herein.

In some embodiments, in Formula 1-1, W₁₁ may be *1, W₁₂ may be *2, W₁₅ may be *3, and W₁₆ may be *4;
in Formula 1-1, W₁₁ may be *1, W₁₂ may be *2, W₁₅ may be *4, and W₁₆ may be *3;
in Formula 1-1, W₁₁ may be *2, W₁₁ may be *1, W₁₅ may be *3, and W₁₆ may be *4;
in Formula 1-2, W₁₃ may be *6, W₁₄ may be *5, W₁₅ may be *8, and W₁₆ may be *7;
in Formula 1-2, W₁₃ may be *6, W₁₄ may be *5, W₁₅ may be *7, and W₁₆ may be *8;
in Formula 1-2, W₁₃ may be *5, W₁₄ may be *6, W₁₅ may be *8, and W₁₆ may be *7; or
in Formula 1-2, W₁₃ may be *6, W₁₄ may be *6, W₁₅ may be *7, and W₁₆ may be *8.

In some embodiments, the heterocyclic compound may be represented by one of Formulae 11 to 17:

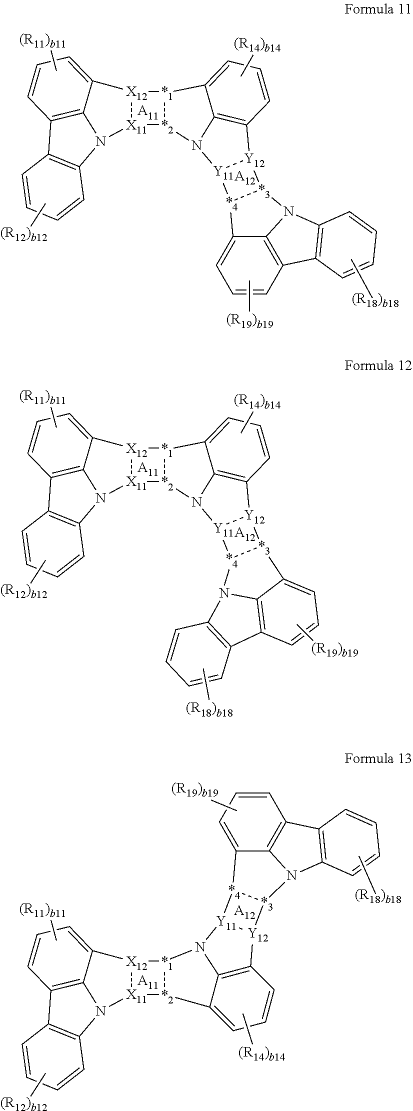

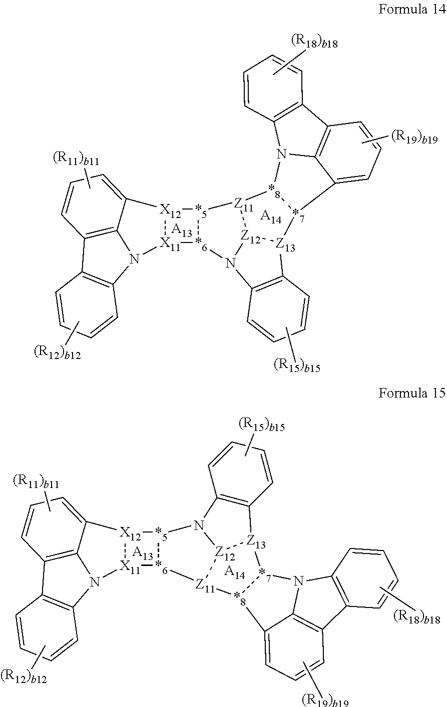

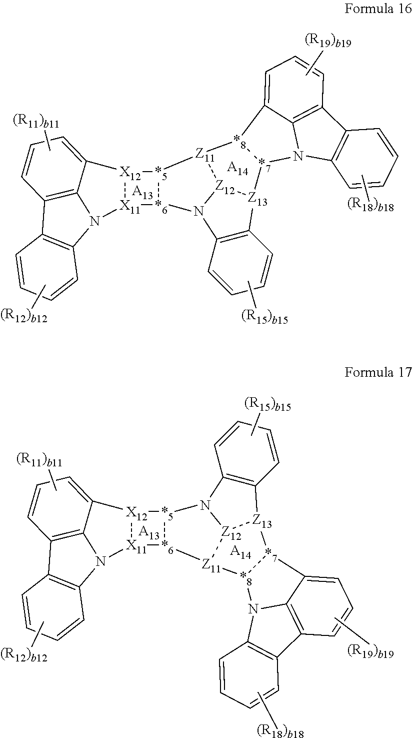

wherein, in Formulae 11 to 17, A₁₁ to A₁₄, R₁₁ to R₁₉, b11 to b19, X₁₁, X₁₂, Y₁₁, Y₁₂, and Z₁₁ to Z₁₃ may respectively be understood by referring to the descriptions of A₁₁ to A₁₄, R₁ to R₁₉, b11 to b19, X₁₁, X₁₂, Y₁₁, Y₁₂, and Z₁₁ to Z₁₃ provided herein.

In Formulae 11, 12, and 14 to 17,

A₁₁ and A₁₃ are each independently a group represented by Formula 2-1,

A₁₂ is a group represented by Formula 2-2,

A₁₄ is a group represented by Formula 2-3,

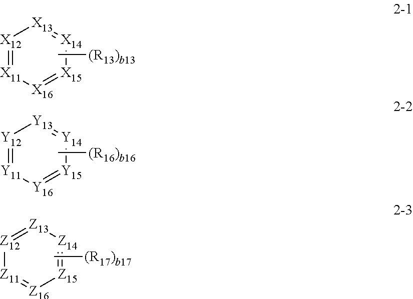

wherein, in Formulae 2-1 to 2-3,

X₁₁ to X₁₆, Y₁₁ to Y₁₆, and Z₁₁ to Z₁₆ are each independently a carbon atom, any suitable two adjacent groups of X₁₃ to X₁₆ are respectively *1 and *2 in Formulae 11 and 12 or *5 and *6 in Formulae 14 to 17, any suitable two adjacent groups of Y₁₃ to Y₁₆ are respectively *3 and *4 in Formulae 11 and 12, and any suitable two adjacent groups of Z₁₄ to Z₁₆ are respectively *7 and *8 in Formulae 14 to 17.

In some embodiments, in Formula 11, i)*1 may be X₁₃, *2 may be X₁₄, *3 may be Y₁₃, and *4 may be Y₁₄; ii)*1 may be X₁₃, *2 may be X₁₄, *3 may be Y₁₄, and *4 may be Y₁₅; iii)*1 may be X₁₃, *2 may be X₁₄, *3 may be Y₁₅, and *4 may be Y₁₆; iv)*1 may be X₁₄, *2 may be X₁₅, *3 may be Y₁₃, and *4 may be Y₁₄; v)*1 may be X₁₄, *2 may be X₁₅, *3 may be Y₁₄, and *4 may be Y₁₅; vi)*1 may be X₁₄, *2 may be X₁₅, *3 may be Y₁₅, and *4 may be Y₁₆; vii)*1 may be X₁₅, *2 may be X₁₆, *3 may be Y₁₃, and *4 may be Y₁₄; viii) *1 may be X₁₅, *2 may be X₁₆, *3 may be Y₁₄, and *4 may be Y₁₅; or ix)*1 may be X₁₅, *2 may be X₁₆, *3 may be Y₁₅, and *4 may be Y₁₆.

In some embodiments, in Formula 12, i)*1 may be X₁₃, *2 may be X₁₄, *3 may be Y₁₃, and *4 may be Y₁₄; ii)*1 may be X₁₃, *2 may be X₁₄, *3 may be Y₁₄, and *4 may be Y₁₅; iii)*1 may be X₁₃, *2 may be X₁₄, *3 may be Y₁₅, and *4 may be Y₁₆; iv)*1 may be X₁₄, *2 may be X₁₅, *3 may be Y₁₄, and *4 may be Y₁₅; v)*1 may be X₁₄, *2 may be X₁₅, *3 may be Y₁₅, and *4 may be Y₁₆; or vi)*1 may be X₁₅, *2 may be X₁₆, *3 may be Y₁₅, and *4 may be Y₁₆.

In some embodiments, in Formula 13, i)*1 may be X₁₃, *2 may be X₁₄, *3 may be Y₁₃, and *4 may be Y₁₄; ii)*1 may be X₁₃, *2 may be X₁₄, *3 may be Y₁₄, and *4 may be Y₁₅; iii)*1 may be X₁₃, *2 may be X₁₄, *3 may be Y₁₅, and *4 may be Y₁₆; iv)*1 may be X₁₄, *2 may be X₁₅, *3 may be Y₁₄, and *4 may be Y₁₅; v)*1 may be X₁₄, *2 may be X₁₅, *3 may be Y₁₅, and *4 may be Y₁₆; or vi)*1 may be X₁₅, *2 may be X₁₆, *3 may be Y₁₅, and *4 may be Y₁₆.

In some embodiments, in Formula 14, i)*5 may be X₁₃, *6 may be X₁₄, *7 may be Z₁₄, and *8 may be Z₁₅; ii)*5 may be X₁₃, *6 may be X₁₄, *7 may be Z₁₅, and *8 may be Z₁₆; iii)*5 may be X₁₄, *6 may be X₁₅, *7 may be Z₁₄, and *8 may be Z₁₅; iv)*5 may be X₁₄, *6 may be X₁₅, *7 may be Z₁₅, and *8 may be Z₁₆; v)*5 may be X₁₅, *6 may be X₁₆, *7 may be Z₁₄, and *8 may be Z₁₅; or vi)*5 may be X₁₅, *6 may be X₁₆, *7 may be Z₁₅, and *8 may be Z₁₆.

In some embodiments, in Formula 15, i)*5 may be X₁₃, *6 may be X₁₄, *7 may be Z₁₄, and *8 may be Z₁₅; ii)*5 may be X₁₃, *6 may be X₁₄, *7 may be Z₁₅, and *8 may be Z₁₆; iii)*5 may be X₁₄, *6 may be X₁₅, *7 may be Z₁₄, and *8 may be Z₁₅; iv)*5 may be X₁₄, *6 may be X₁₅, *7 may be Z₁₅, and *8 may be Z₁₆; v)*5 may be X₁₅, *6 may be X₁₆, *7 may be Z₁₄, and *8 may be Z₁₅; or vi)*5 may be X₁₅, *6 may be X₁₆, *7 may be Z₁₅, and *8 may be Z₁₆.

In some embodiments, in Formula 16, i)*5 may be X₁₃, *6 may be X₁₄, *7 may be Z₁₄, and *8 may be Z₁₅; ii)*5 may be X₁₃, *6 may be X₁₄, *7 may be Z₁₅, and *8 may be Z₁₆; iii)*5 may be X₁₄, *6 may be X₁₅, *7 may be Z₁₄, and *8 may be Z₁₅; iv)*5 may be X₁₄, *6 may be X₁₅, *7 may be Z₁₅, and *8 may be Z₁₆; v)*5 may be X₁₅, *6 may be X₁₆, *7 may be Z₁₄, and *8 may be Z₁₅; or vi)*5 may be X₁₅, *6 may be X₁₆, *7 may be Z₁₅, and *8 may be Z₁₆.

In some embodiments, in Formula 17, i)*5 may be X₁₃, *6 may be X₁₄, *7 may be Z₁₄, and *8 may be Z₁₅; ii)*5 may be X₁₃, *6 may be X₁₄, *7 may be Z₁₅, and *8 may be Z₁₆; iii)*5 may be X₁₄, *6 may be X₁₅, *7 may be Z₁₄, and *8 may be Z₁₅; iv)*5 may be X₁₄, *6 may be X₁₅, *7 may be Z₁₅, and *8 may be Z₁₆; v)*5 may be X₁₅, *6 may be X₁₆, *7 may be Z₁₄, and *8 may be Z₁₅; or vi)*5 may be X₁₅, *6 may be X₁₆, *7 may be Z₁₅, and *8 may be Z₁₆.

In some embodiments, the heterocyclic compound may be represented by one of Formulae 11-1 to 11-9, 12-1 to 12-6, 13-1 to 13-5, 14-1 to 14-6, 15-1 to 15-6, 16-1 to 16-6, and 17-1 to 17-6:

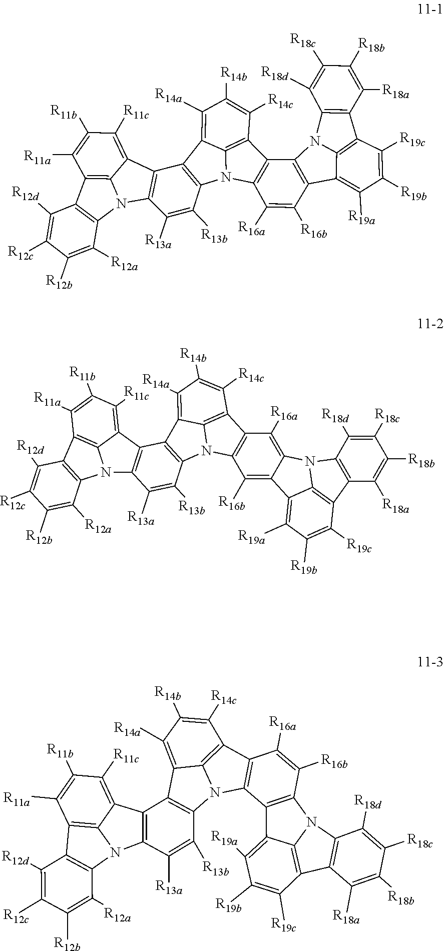

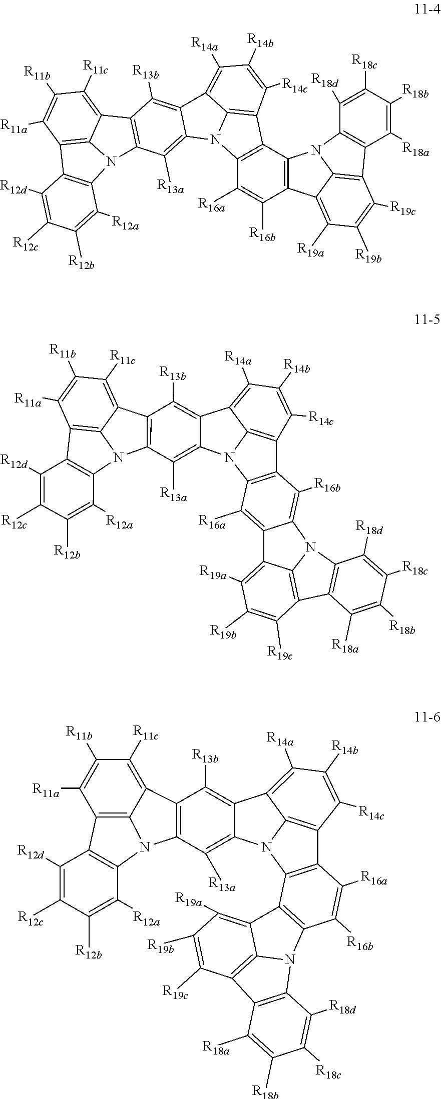

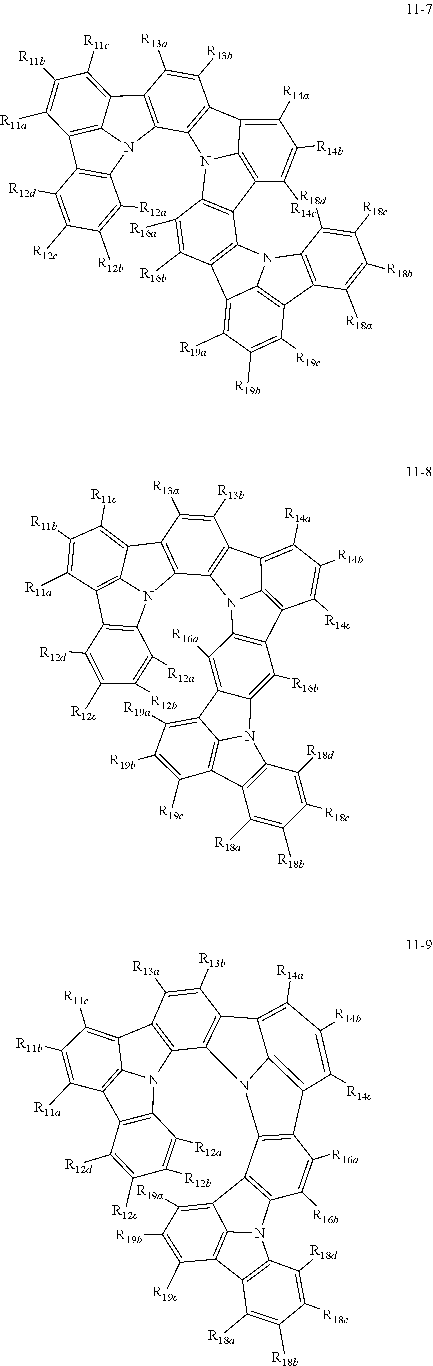

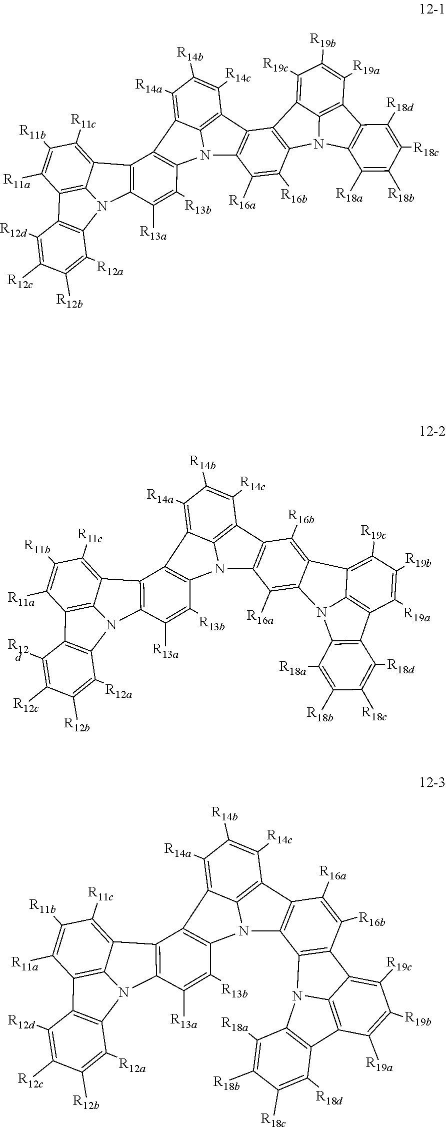

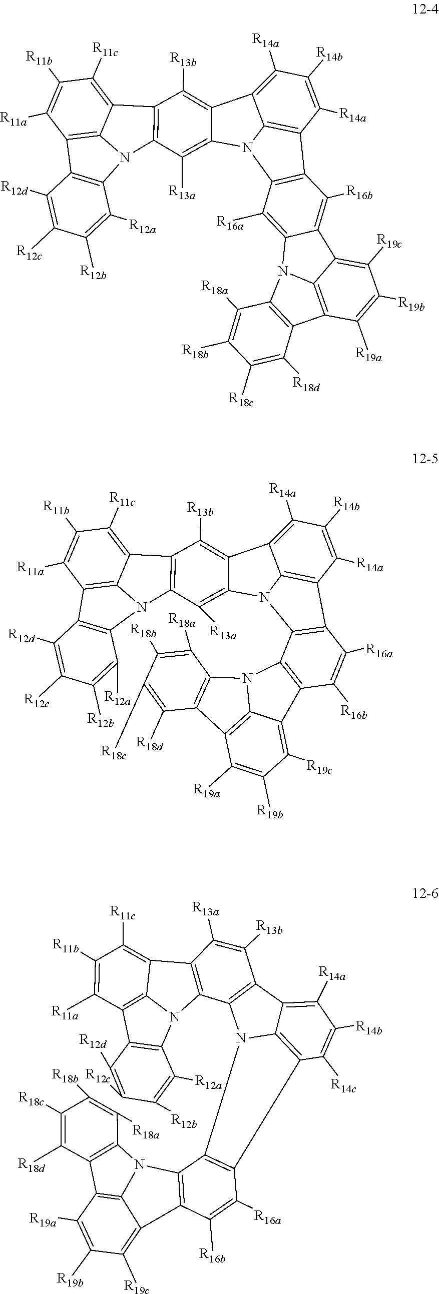

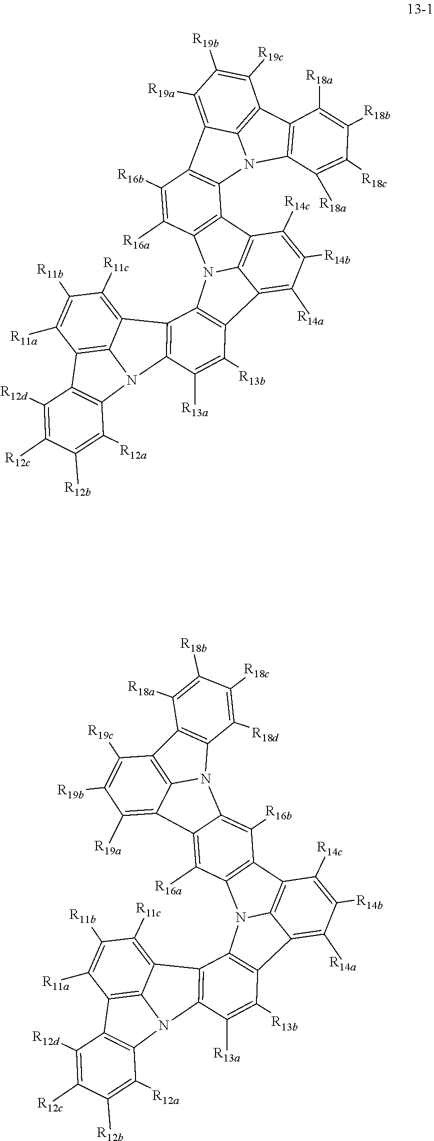

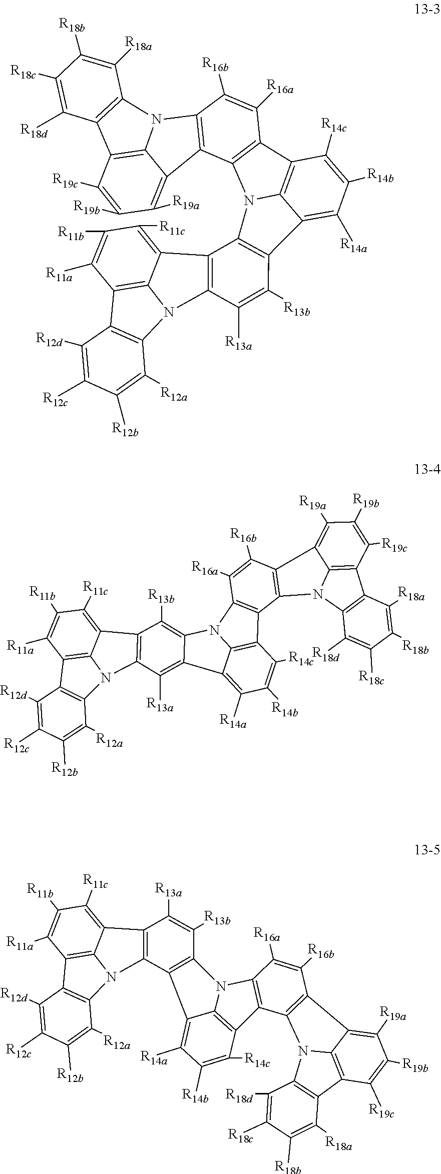

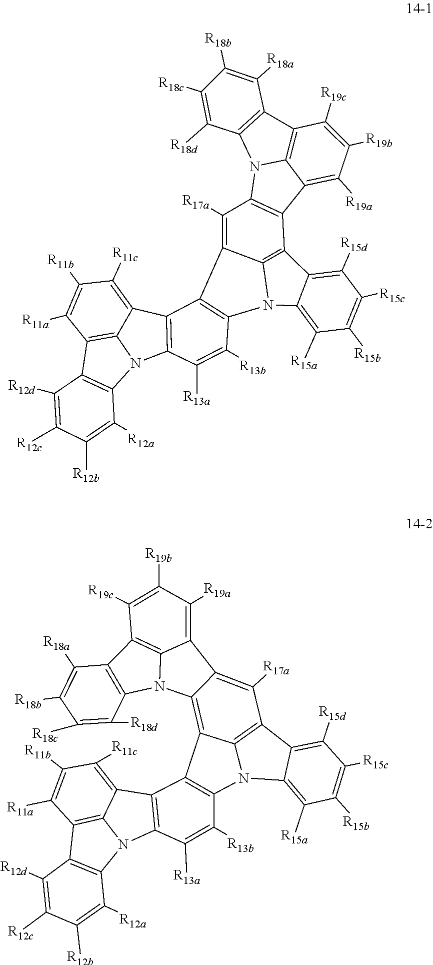

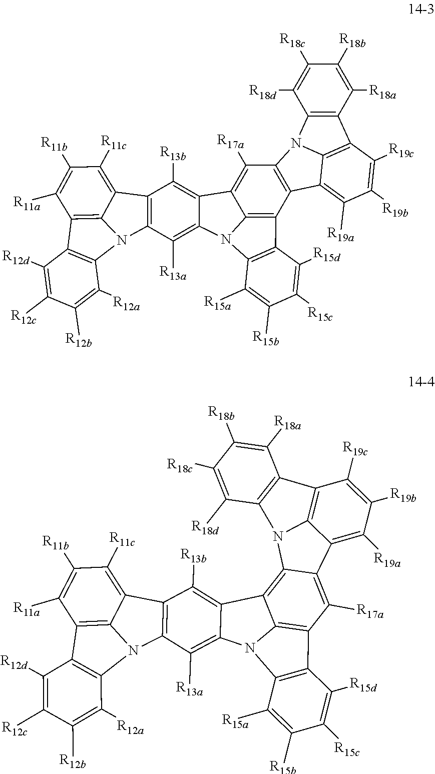

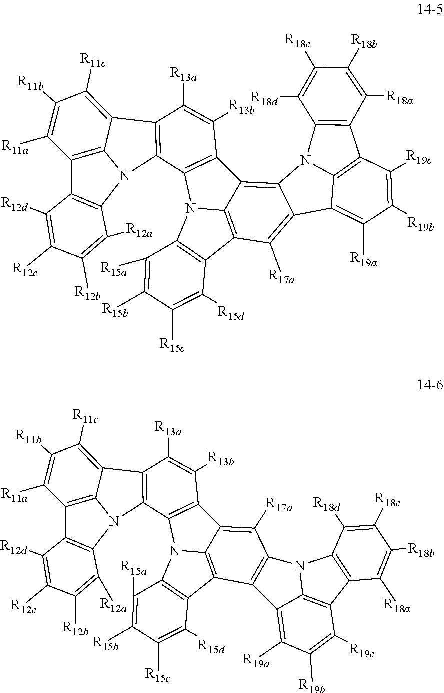

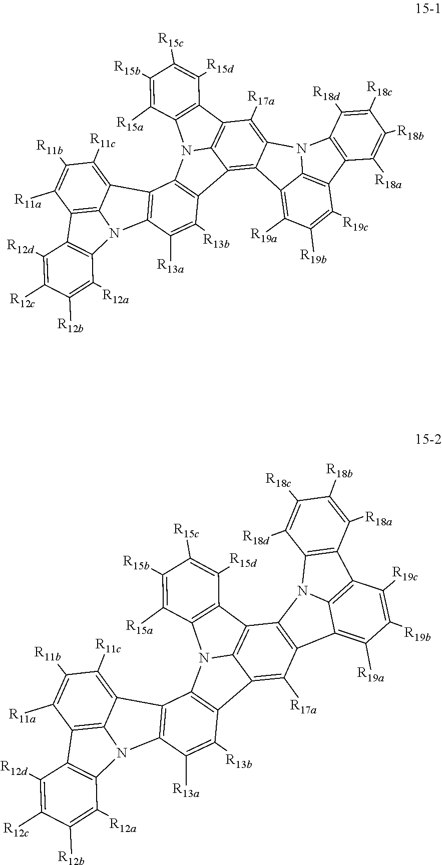

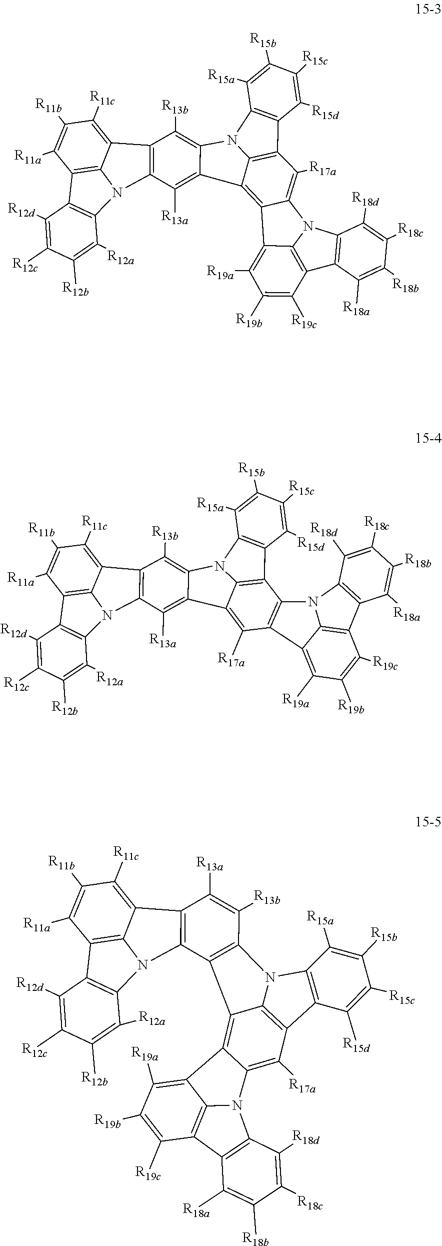

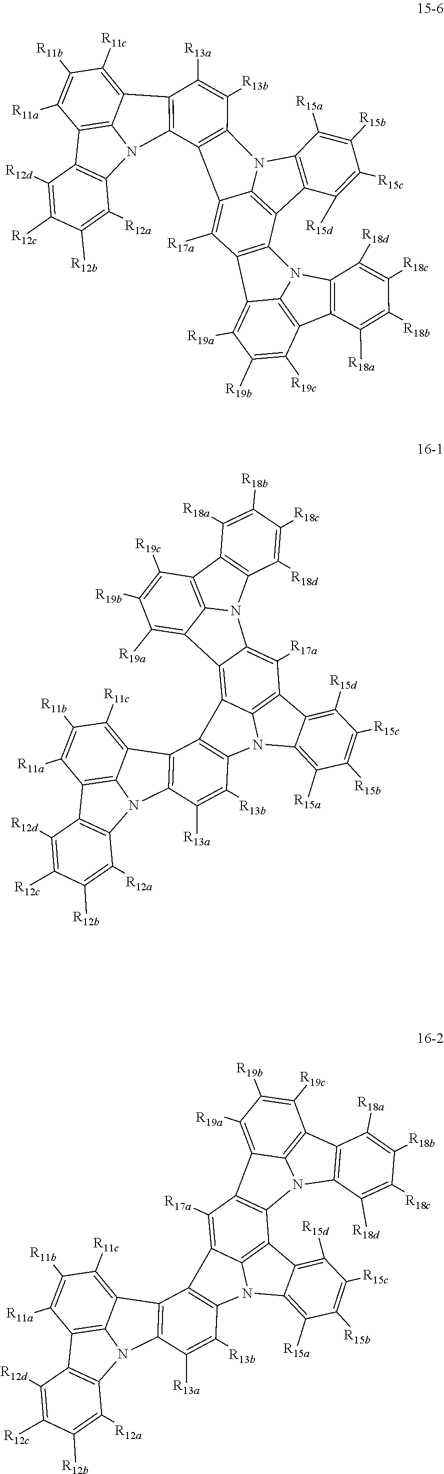

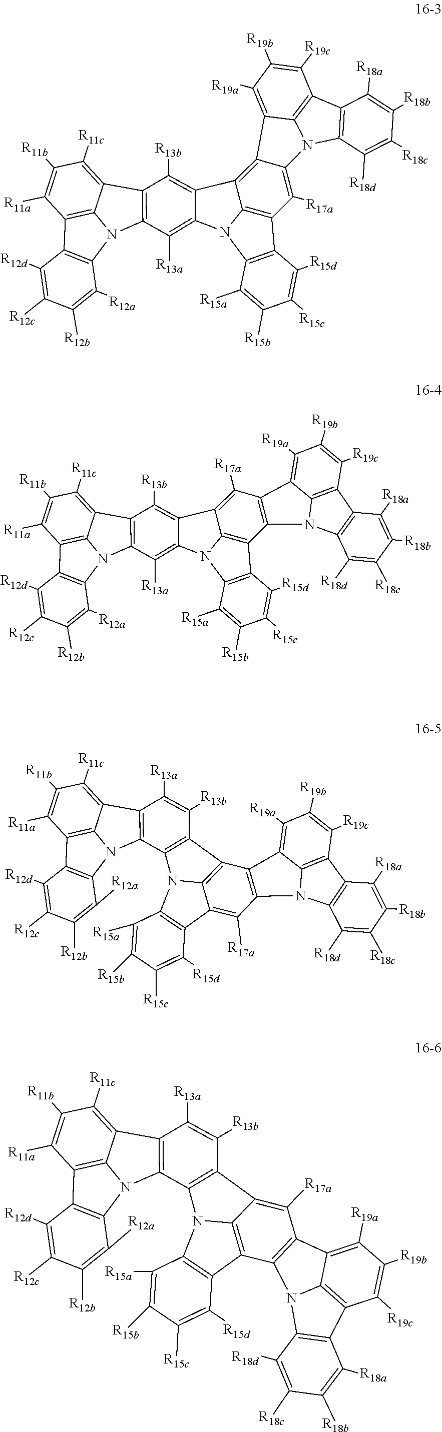

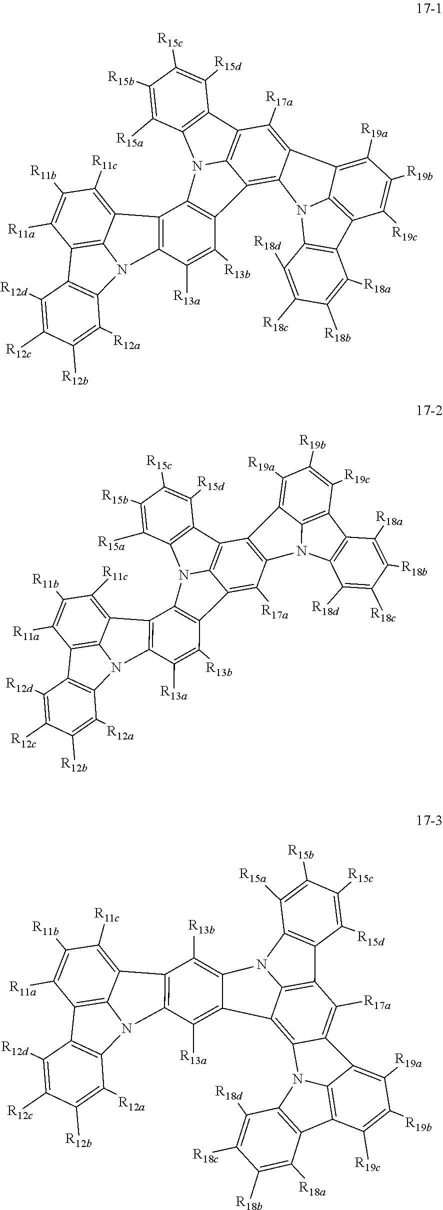

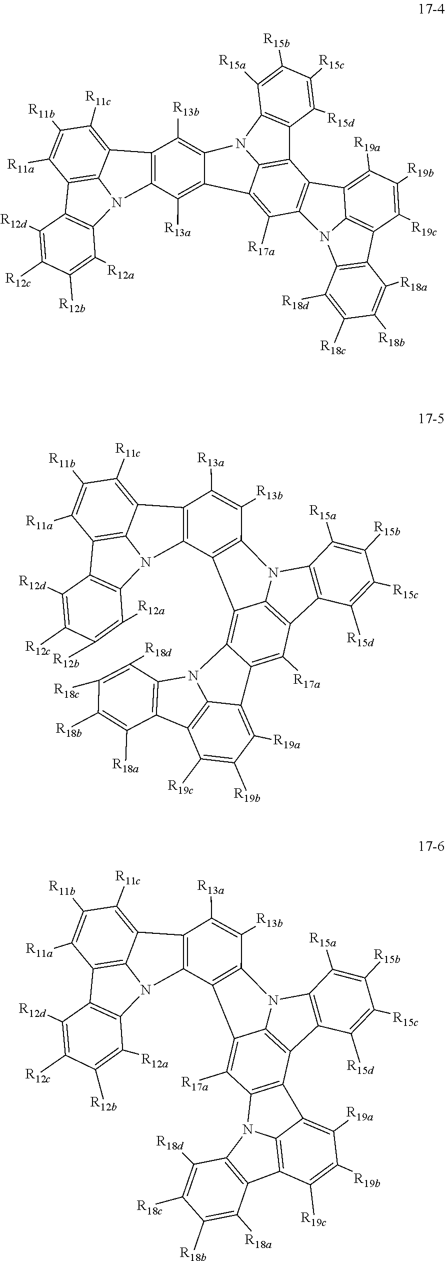

wherein, in Formulae 11-1 to 11-9, 12-1 to 12-6, 13-1 to 13-5, 14-1 to 14-6, 15-1 to 15-6, 16-1 to 16-6, and 17-1 to 17-6,

R₁₁ₐ to R_{11c} may each be understood by referring to the description of R₁₁ provided herein,

R₁₂ₐ to R_{12d} may each be understood by referring to the description of R₁₂ provided herein,

R₁₃ₐ and R_{13b} may each be understood by referring to the description of R₁₃ provided herein,

R₁₄ₐ to R_{14c} may each be understood by referring to the description of R₁₄ provided herein,

R₁₅ₐ to R_{15d} may each be understood by referring to the description of R₁₅ provided herein,

R₁₆ₐ and R_{16b} may each be understood by referring to the description of R₁₆ provided herein,

R₁₇ₐ may be understood by referring to the description of R₁₇ provided herein,

R₁₈ₐ to R₁₈ₐ may each be understood by referring to the description of R₁₈ provided herein, and

R₁₉ₐ to R_{19c} may each be understood by referring to the description of R₁₉ provided herein.

In some embodiments, at least one of R_{11b}, R_{12c}, R_{14b}, R_{18b}, and R_{19b} in Formulae 11-1, 11-2, and 11-5, at least one of R_{11b}, R_{12c}, R_{14b}, R_{16b}, R_{18b}, and R_{19b} in Formulae 11-3 and 11-6, at least one of R₁₁, R_{12c}, R_{14b}, R₁₆ₐ, R_{18b}, and R_{19b} in Formula 11-4, at least one of R_{11b}, R_{12c}, R₁₃ₐ, R_{13b}, R_{14b}, R_{18b} and R_{19b} in Formulae 11-7, 11-8, and 11-9, at least one of R_{11b}, R_{12c}, R_{14b}, R_{18c}, and R_{19b} in Formulae 12-1, 12-2, and 12-4, at least one of R_{11b}, R_{12c}, R_{14b}, R_{16b}, R_{18c}, and R_{19b} in Formula 12-3, at least one of R_{11b}, R_{12c}, R_{14b}, R₁₆ₐ, R_{16b}, R_{18c}, and R_{19b} in Formula 12-5, at least one of R_{11b}, R_{12c}, R₁₃ₐ, R_{13b}, R_{14b}, R₁₆ₐ, R_{16b}, R_{18c}, and R_{19b} in Formulae 12-6, 13-1, and 13-3, at least one of R_{11b}, R_{12c}, R₁₃ₐ, R_{13b}, R_{14b}, R_{18c}, and R_{19b} in Formula 13-2, at least one of R_{11b}, R_{12c}, R_{14b}, R₁₆ₐ, R_{18c}, and R_{19b} in Formula 13-4, at least one of R_{11b}, R_{12c}, R_{13b}, R_{14b}, R₁₆ₐ, R_{18c}, and R_{19b} in Formula 13-5, at least one of R_{11b}, R_{12c}, R_{15c}, R_{18b}, and R_{19b} in Formulae 14-1, 14-2, 14-3, 14-4, 15-3, 15-4, 16-1, 16-2, 16-3, 16-4, 17-3, and 17-4, at least one of R_{11b}, R_{12c}, R₁₃ₐ, R_{13b}, R_{15c}, R_{18b}, and R_{19b} in Formulae 14-5, 14-6, 16-5, and 16-6, at least one of R_{11b}, R_{12c}, R₁₃ₐ, R_{15c}, R_{18b}, and R_{19b} in Formulae 15-1, 15-2, 17-1, and 17-2, and at least one of R_{11b}, R_{12c}, R_{13b}, R_{15c}, R_{18b}, and R_{19b} in Formulae 15-5, 15-6, 17-5, and 17-6 may be —CH₃, —CD₃, —CD₂H, —CDH₂, —CF₃, —CF₂H, —CFH₂, a group represented by one of Formulae 9-1 to 9-39, a group represented by one of Formulae 9-1 to 9-39 in which at least one hydrogen is substituted with deuterium, a group represented by one of Formulae 9-1 to 9-39 in which at least one hydrogen is substituted with —F, a group represented by one of Formulae 10-1 to 10-130, a group represented by one of Formulae 10-1 to 10-130 in which at least one hydrogen is substituted with deuterium, a group represented by one of Formulae 10-1 to 10-130 in which at least one hydrogen is substituted with —F, —Si(Q₁)(Q₂)(Q₃), —Ge(Q₁)(Q₂)(Q₃), —C(Q₁)(Q₂)(Q₃), —B(Q₁)(Q₂), or —N(Q₁)(Q₂),

wherein Q₁ to Q₃ may each independently be:

deuterium, —F, —CH₃, —CD₃, —CD₂H, —CDH₂, —CH₂CH₃, —CH₂CD₃, —CH₂CD₂H, —CH₂CDH₂, —CHDCH₃, —CHDCD₂H, —CHDCDH₂, —CHDCD₃, —CD₂CD₃, —CD₂CD₂H, —CD₂CDH₂, —CF₃, —CF₂H, —CFH₂, —CH₂CF₃, —CH₂CF₂H, —CH₂CFH₂, —CHFCH₃, —CHFCF₂H, —CHFCFH₂, —CHFCF₃, —CF₂CF₃, —CF₂CF₂H, or —CF₂CFH₂; or

an n-propyl group, an iso-propyl group, an n-butyl group, a sec-butyl group, an isobutyl group, a tert-butyl group, an n-pentyl group, a tert-pentyl group, a neopentyl group, an isopentyl group, a sec-pentyl group, a 3-pentyl group, a sec-isopentyl group, a phenyl group, a biphenyl group, or a naphthyl group, each unsubstituted or substituted with deuterium, —F, a C₁-C₁₀ alkyl group, a phenyl group, or any combination thereof.

In some embodiments, at least one of R_{11b}, R_{12c}, R_{14b}, R_{18b}, and R_{19b} in Formulae 11-1, 11-2, and 11-5, at least one of R_{11b}, R_{12c}, R_{14b}, R_{16b}, R_{18b}, and R_{19b} in Formulae 11-3 and 11-6, at least one of R_{11b}, R_{12c}, R_{14b}, R₁₆ₐ, R_{18b}, and R_{19b} in Formula 11-4, at least one of R_{11b}, R_{12c}, R₁₃ₐ, R_{13b}, R_{14b}, R_{18b} and R_{19b} in Formulae 11-7, 11-8, and 11-9, at least one of R_{11b}, R_{12c}, R_{14b}, R_{18c}, and R_{19b} in Formulae 12-1, 12-2, and 12-4, at least one of R_{11b}, R_{12c}, R_{14b}, R_{16b}, R_{18c}, and R_{19b} in Formula 12-3, at least one of R_{11b}, R_{12c}, R_{14b}, R₁₆ₐ, R_{16b}, R_{18c}, and R_{19b} in Formula 12-5, at least one of R_{11b}, R_{12c}, R₁₃ₐ, R_{13b}, R_{14b}, R₁₆ₐ, R_{16b}, R_{18c}, and R_{19b} in Formulae 12-6, 13-1, and 13-3, at least one of R_{11b}, R_{12c}, R₁₃ₐ, R_{13b}, R_{14b}, R_{18c}, and R_{19b} in Formula 13-2, at least one of R_{11b}, R_{12c}, R_{14b}, R₁₆ₐ, R_{18c}, and R_{19b} in Formula 13-4, at least one of R_{11b}, R_{12c}, R_{13b}, R_{14b}, R₁₆ₐ, R_{18c}, and R_{19b} in Formula 13-5, at least one of R_{11b}, R_{12c}, R_{15c}, R_{18b}, and R_{19b} in Formulae 14-1, 14-2, 14-3, 14-4, 15-3, 15-4, 16-1, 16-2, 16-3, 16-4, 17-3, and 17-4, at least one of R_{11b}, R_{12c}, R₁₃ₐ, R_{13b}, R_{15c}, R_{18b}, and R_{19b} in Formulae 14-5, 14-6, 16-5, and 16-6, at least one of R_{11b}, R_{12c}, R₁₃ₐ, R_{15c}, R_{18b}, and R_{19b} in Formulae 15-1, 15-2, 17-1, and 17-2, and at least one of R_{11b}, R_{12c}, R_{13b}, R_{15c}, R_{18b}, and R_{19b} in Formulae 15-5, 15-6, 17-5, and 17-6 may be —CH₃, —CD₃, —CD₂H, —CDH₂, —CF₃, —CF₂H, —CFH₂, a group represented by one of Formulae 9-1 to 9-39, a group represented by one of Formulae 9-1 to 9-39 in which at least one hydrogen is substituted with deuterium, a group represented by one of Formulae 9-1 to 9-39 in which at least one hydrogen is substituted with —F, a group represented by one of Formulae 10-1 to 10-130, a group represented by one of Formulae 10-1 to 10-130 in which at least one hydrogen is substituted with deuterium, a group represented by one of Formulae 10-1 to 10-130 in which at least one hydrogen is substituted with —F, —Si(Q₁)(Q₂)(Q₃), —Ge(Q₁)(Q₂)(Q₃), —C(Q₁)(Q₂)(Q₃), —B(Q₁)(Q₂), or —N(Q₁)(Q₂), wherein Q₁ to Q₃ may each independently be: deuterium, —F, —CH₃, —CD₃, —CD₂H, —CDH₂, —CH₂CH₃, —CH₂CD₃, —CH₂CD₂H, —CH₂CDH₂, —CHDCH₃, —CHDCD₂H, —CHDCDH₂, —CHDCD₃, —CD₂CD₃, —CD₂CD₂H, —CD₂CDH₂, —CF₃, —CF₂H, —CFH₂, —CH₂CF₃, —CH₂CF₂H, —CH₂CFH₂, —CHFCH₃, —CHFCF₂H, —CHFCFH₂, —CHFCF₃, —CF₂CF₃, —CF₂CF₂H, or —CF₂CFH₂; or an n-propyl group, an iso-propyl group, an n-butyl group, a sec-butyl group, an isobutyl group, a tert-butyl group, an n-pentyl group, a tert-pentyl group, a neopentyl group, an isopentyl group, a sec-pentyl group, a 3-pentyl group, a sec-isopentyl group, a phenyl group, a biphenyl group, or a naphthyl group, each unsubstituted or substituted with deuterium, —F, a C₁-C₁₀ alkyl group, a phenyl group, or any combination thereof, and the remaining substituents may each be hydrogen.

In some embodiments, at least one of R_{11b}, R_{12c}, R_{14b}, R_{18b}, and R_{19b} in Formulae 11-1, 11-2, and 11-5, at least one of R_{11b}, R_{12c}, R_{14b}, R_{16b}, R_{18b}, and R_{19b} in Formulae 11-3 and 11-6, at least one of R₁, R_{12c}, R_{14b}, R₁₆ₐ, R_{18b}, and R_{19b} in Formula 11-4, at least one of R_{11b}, R_{12c}, R₁₃ₐ, R_{13b}, R_{14b}, R_{18b}, and R_{19b} in Formulae 11-7, 11-8, and 11-9, at least one of R_{11b}, R_{12c}, R_{14b}, R_{18c}, and R_{19b} in Formulae 12-1, 12-2, and 12-4, at least one of R_{11b}, R_{12c}, R_{14b}, R_{16b}, R_{18c}, and R_{19b} in Formula 12-3, at least one of R_{11b}, R_{12c}, R_{14b}, R₁₆ₐ, R_{16b}, R_{18c}, and R_{19b} in Formula 12-5, at least one of R_{11b}, R_{12c}, R₁₃ₐ, R_{13b}, R_{14b}, R₁₆ₐ, R_{16b}, R_{18c}, and R_{19b} in Formulae 12-6, 13-1, and 13-3, at least one of R_{11b}, R_{12c}, R₁₃ₐ, R_{13b}, R_{14b}, R_{18c}, and R_{19b} in Formula 13-2, at least one of R_{11b}, R_{12c}, R_{14b}, R₁₆ₐ, R_{18c}, and R_{19b} in Formula 13-4, at least one of R_{11b}, R_{12c}, R_{13b}, R_{14b}, R₁₆ₐ, R_{18c}, and R_{19b} in Formula 13-5, at least one of R_{11b}, R_{12c}, R_{15c}, R_{18b}, and R_{19b} in Formulae 14-1, 14-2, 14-3, 14-4, 15-3, 15-4, 16-1, 16-2, 16-3, 16-4, 17-3, and 17-4, at least one of R_{11b}, R_{12c}, R₁₃ₐ, R_{13b}, R_{15c}, R_{18b}, and R_{19b} in Formulae 14-5, 14-6, 16-5, and 16-6, at least one of R_{11b}, R_{12c}, R₁₃ₐ, R_{15c}, R_{18b}, and R_{19b} in Formulae 15-1, 15-2, 17-1, and 17-2, and at least one of R_{11b}, R_{12c}, R_{13b}, R_{15c}, R_{18b}, and R_{19b} in Formulae 15-5, 15-6, 17-5, and 17-6 may each be Formulae A-1 to A-29, and

the remaining substituents may each be hydrogen:

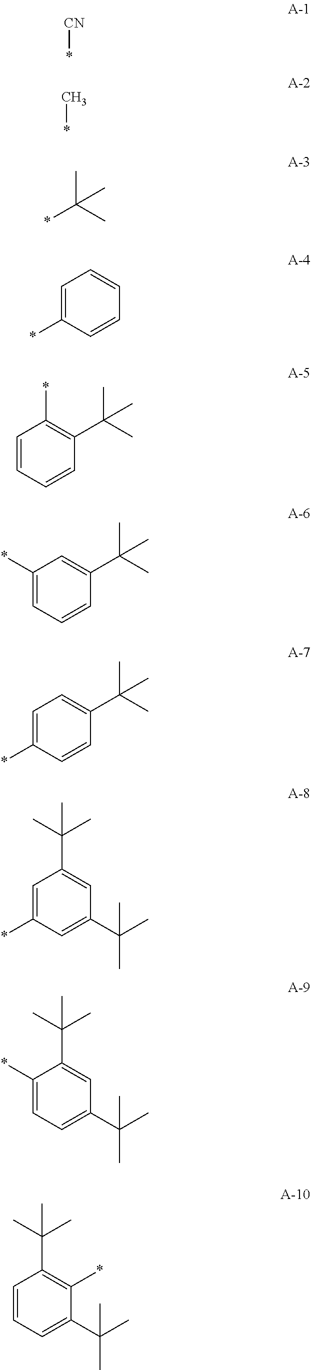

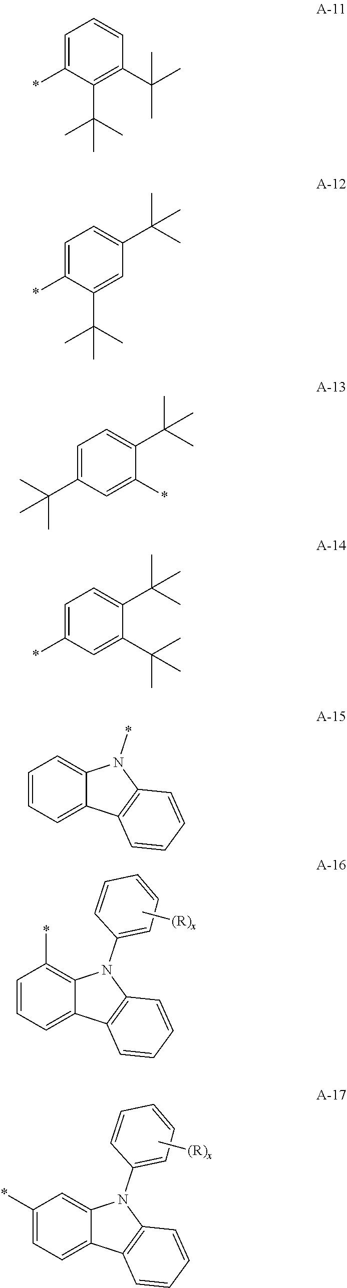

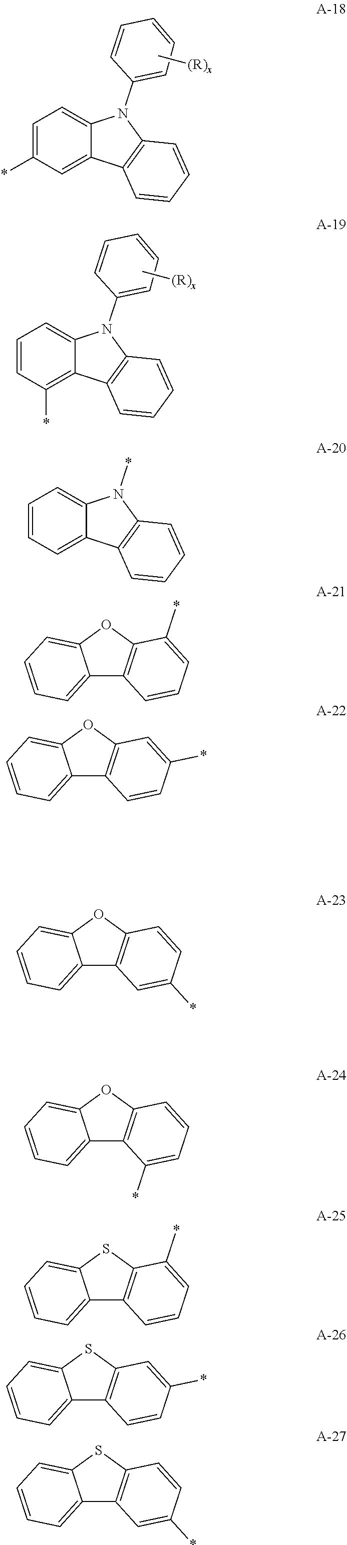

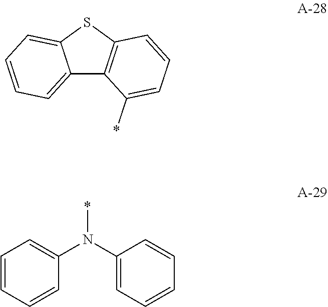

wherein, in Formulae A-1 to A-29,

R may be hydrogen, CN, a methyl group, a t-butyl group, or a phenyl group,

x may be an integer from 1 to 5, and

* indicates a binding site to an adjacent atom.

In some embodiments, the heterocyclic compound may be of Groups HCl to HC10: Group HCl

The heterocyclic compound may be represented by Formula 11-2, and substituents may be defined as shown in Table 1.

TABLE 1

| Compound | | | | | |
|---|---|---|---|---|---|
| No. | R_{11b} | R_{12c} | R_{14b} | R_{18b} | R_{19b} |
| 112001 | t-Bu | t-Bu | t-Bu | t-Bu | t-Bu |
| 112002 | Ph | Ph | t-Bu | Ph | Ph |
| 112003 | t-Bu | t-Bu | H | H | H |
| 112004 | H | H | t-Bu | H | H |
| 112005 | H | H | H | t-Bu | t-Bu |
| 112006 | t-Bu | t-Bu | t-Bu | H | H |
| 112007 | H | H | t-Bu | t-Bu | t-Bu |
| 112008 | t-Bu | t-Bu | H | t-Bu | t-Bu |
| 112009 | Ph | Ph | H | H | H |
| 112010 | H | H | Ph | H | H |
| 112011 | H | H | H | Ph | Ph |
| 112012 | Ph | Ph | Ph | H | H |
| 112013 | H | H | Ph | Ph | Ph |
| 112014 | Ph | Ph | H | Ph | Ph |
| 112015 | CN | CN | H | H | H |
| 112016 | H | H | CN | H | H |
| 112017 | H | H | H | CN | CN |
| 112018 | CN | CN | CN | H | H |
| 112019 | H | H | CN | CN | CN |
| 112020 | CN | CN | H | CN | CN |
| 112021 | 9-Cbz | 9-Cbz | H | H | H |
| 112022 | H | H | 9- | H | H |
| | | | Cbz | | |
| 112023 | H | H | H | 9- | 9- |
| | | | | Cbz | Cbz |
| 112024 | 9-Cbz | 9-Cbz | 9- | H | H |
| | | | Cbz | | |
| 112025 | H | H | 9- | 9- | 9- |
| | | | Cbz | Cbz | Cbz |
| 112026 | 9-Cbz | 9-Cbz | H | 9- | 9- |
| | | | | Cbz | Cbz |
| 112027 | Me | Me | H | H | H |
| 112028 | H | H | Me | H | H |
| 112029 | H | H | H | Me | Me |
| 112030 | Me | Me | Me | H | H |
| 112031 | H | H | Me | Me | Me |
| 112032 | Me | Me | H | Me | Me |
| 112033 | CD₃ | CD3 | H | H | H |
| 112034 | H | H | CD3 | H | H |
| 112035 | H | H | H | CD3 | CD3 |
| 112036 | CD3 | CD3 | CD3 | H | H |
| 112037 | H | H | CD3 | CD3 | CD3 |
| 112038 | CD3 | CD3 | H | CD3 | CD3 |
| 112039 | 4-t- | 4-t- | H | H | H |
| | BuPh | BuPh | | | |
| 112040 | H | H | 4-t- | H | H |
| | | | BuPh | | |
| 112041 | H | H | H | 4-t- | 4-t- |
| | | | | BuPh | BuPh |
| 112042 | 4-t- | 4-t- | 4-t- | H | H |
| | BuPh | BuPh | BuPh | | |
| 112043 | H | H | 4-t- | 4-t- | 4-t- |
| | | | BuPh | BuPh | BuPh |
| 112044 | 4-t- | 4-t- | H | 4-t- | 4-t- |
| | BuPh | BuPh | | BuPh | BuPh |
| 112045 | 1,3- | 1,3-di-t- | H | H | H |
| | di-t- | BuPh | | | |
| | BuPh | | | | |
| 112046 | H | H | 1,3- | H | H |
| | | | di-t- | | |
| | | | BuPh | | |
| 112047 | H | H | H | 1,3- | 1,3- |
| | | | | di-t- | di-t- |
| | | | | BuPh | BuPh |
| 112048 | 1,3- | 1,3-di-t- | 1,3- | H | H |
| | di-t- | BuPh | di-t- | | |
| | BuPh | | BuPh | | |
| 112049 | H | H | 1,3- | 1,3- | 1,3- |
| | | | di-t- | di-t- | di-t- |
| | | | BuPh | BuPh | BuPh |
| 112050 | 1,3- | 1,3-di-t- | H | 1,3- | 1,3- |
| | di-t- | BuPh | | di-t- | di-t- |
| | BuPh | | | BuPh | BuPh |
| 112051 | t-Bu | t-Bu | Ph | H | H |
| 112052 | t-Bu | t-Bu | CN | H | H |
| 112053 | t-Bu | t-Bu | DPA | H | H |
| 112054 | t-Bu | t-Bu | 2- | H | H |
| | | | DBF | | |
| 112055 | t-Bu | t-Bu | 2- | H | H |
| | | | DBT | | |
| 112056 | t-Bu | t-Bu | 4-t- | H | H |
| | | | BuPh | | |
| 112057 | t-Bu | t-Bu | 1,3- | H | H |
| | | | di-t- | | |
| | | | BuPh | | |
| 112058 | t-Bu | t-Bu | 9- | H | H |
| | | | Cbz | | |
| 112059 | t-Bu | t-Bu | Ph | t-Bu | t-Bu |
| 112060 | t-Bu | t-Bu | CN | t-Bu | t-Bu |
| 112061 | t-Bu | t-Bu | DPA | t-Bu | t-Bu |
| 112062 | t-Bu | t-Bu | 2- | t-Bu | t-Bu |
| | | | DBF | | |
| 112063 | t-Bu | t-Bu | 2- | t-Bu | t-Bu |
| | | | DBT | | |
| 112064 | t-Bu | t-Bu | 4-t- | t-Bu | t-Bu |
| | | | BuPh | | |
| 112065 | t-Bu | t-Bu | 1,3- | t-Bu | t-Bu |
| | | | di-t- | | |
| | | | BuPh | | |
| 112066 | t-Bu | t-Bu | 9- | t-Bu | t-Bu |
| | | | Cbz | | |
| 112067 | t-Bu | t-Bu | t-Bu | Ph | Ph |
| 112068 | t-Bu | t-Bu | Ph | Ph | Ph |
| 112069 | t-Bu | t-Bu | CN | Ph | Ph |
| 112070 | t-Bu | t-Bu | DPA | Ph | Ph |
| 112071 | t-Bu | t-Bu | 2- | Ph | Ph |
| | | | DBF | | |
| 112072 | t-Bu | t-Bu | 2- | Ph | Ph |
| | | | DBT | | |
| 112073 | t-Bu | t-Bu | 4-t- | Ph | Ph |
| | | | BuPh | | |
| 112074 | t-Bu | t-Bu | 1,3- | Ph | Ph |
| | | | di-t- | | |
| | | | BuPh | | |
| 112075 | t-Bu | t-Bu | 9- | Ph | Ph |
| | | | Cbz | | |
| 112076 | t-Bu | t-Bu | t-Bu | CN | CN |
| 112077 | t-Bu | t-Bu | Ph | CN | CN |
| 112078 | t-Bu | t-Bu | CN | CN | CN |
| 112079 | t-Bu | t-Bu | DPA | CN | CN |
| 112080 | t-Bu | t-Bu | 2- | CN | CN |
| | | | DBF | | |
| 112081 | t-Bu | t-Bu | 2- | CN | CN |
| | | | DBT | | |
| 112082 | t-Bu | t-Bu | 4-t- | CN | CN |
| | | | BuPh | | |
| 112083 | t-Bu | t-Bu | 1,3- | CN | CN |
| | | | di-t- | | |
| | | | BuPh | | |
| 112084 | t-Bu | t-Bu | 9- | CN | CN |
| | | | Cbz | | |
| 112085 | t-Bu | t-Bu | t-Bu | 9- | 9- |
| | | | | Cbz | Cbz |
| 112086 | t-Bu | t-Bu | Ph | 9- | 9- |
| | | | | Cbz | Cbz |
| 112087 | t-Bu | t-Bu | CN | 9- | 9- |
| | | | | Cbz | Cbz |
| 112088 | t-Bu | t-Bu | DPA | 9- | 9- |
| | | | | Cbz | Cbz |
| 112089 | t-Bu | t-Bu | 2- | 9- | 9- |
| | | | DBF | Cbz | Cbz |
| 112090 | t-Bu | t-Bu | 2- | 9- | 9- |
| | | | DBT | Cbz | Cbz |
| 112091 | t-Bu | t-Bu | 4-t- | 9- | 9- |
| | | | BuPh | Cbz | Cbz |
| 112092 | t-Bu | t-Bu | 1,3- | 9- | 9- |
| | | | di-t- | Cbz | Cbz |
| | | | BuPh | | |
| 112093 | t-Bu | t-Bu | 9- | 9- | 9- |
| | | | Cbz | Cbz | Cbz |
| 112094 | t-Bu | t-Bu | t-Bu | 4-t- | 4-t- |
| | | | | BuPh | BuPh |
| 112095 | t-Bu | t-Bu | Ph | 4-t- | 4-t- |
| | | | | BuPh | BuPh |
| 112096 | t-Bu | t-Bu | CN | 4-t- | 4-t- |
| | | | | BuPh | BuPh |
| 112097 | t-Bu | t-Bu | DPA | 4-t- | 4-t- |
| | | | | BuPh | BuPh |
| 112098 | t-Bu | t-Bu | 2- | 4-t- | 4-t- |
| | | | DBF | BuPh | BuPh |
| 112099 | t-Bu | t-Bu | 2- | 4-t- | 4-t- |
| | | | DBT | BuPh | BuPh |
| 112100 | t-Bu | t-Bu | 4-t- | 4-t- | 4-t- |
| | | | BuPh | BuPh | BuPh |
| 112101 | t-Bu | t-Bu | 1,3- | 4-t- | 4-t- |
| | | | di-t- | BuPh | BuPh |
| | | | BuPh | | |
| 112102 | t-Bu | t-Bu | 9- | 4-t- | 4-t- |
| | | | Cbz | BuPh | BuPh |
| 112103 | t-Bu | t-Bu | t-Bu | 1,3- | 1,3- |
| | | | | di-t- | di-t- |
| | | | | BuPh | BuPh |
| 112104 | t-Bu | t-Bu | Ph | 1,3- | 1,3- |
| | | | | di-t- | di-t- |
| | | | | BuPh | BuPh |
| 112105 | t-Bu | t-Bu | CN | 1,3- | 1,3- |
| | | | | di-t- | di-t- |
| | | | | BuPh | BuPh |
| 112106 | t-Bu | t-Bu | DPA | 1,3- | 1,3- |
| | | | | di-t- | di-t- |
| | | | | BuPh | BuPh |
| 112107 | t-Bu | t-Bu | 2- | 1,3- | 1,3- |
| | | | DBF | di-t- | di-t- |
| | | | | BuPh | BuPh |
| 112108 | t-Bu | t-Bu | 2- | 1,3- | 1,3- |
| | | | DBT | di-t- | di-t- |
| | | | | BuPh | BuPh |
| 112109 | t-Bu | t-Bu | 4-t- | 1,3- | 1,3- |
| | | | BuPh | di-t- | di-t- |
| | | | | BuPh | BuPh |
| 112110 | t-Bu | t-Bu | 1,3- | 1,3- | 1,3- |
| | | | di-t- | di-t- | di-t- |
| | | | BuPh | BuPh | BuPh |
| 112111 | t-Bu | t-Bu | 9- | 1,3- | 1,3- |
| | | | Cbz | di-t- | di-t- |
| | | | | BuPh | BuPh |
| 112112 | Ph | Ph | t-Bu | H | H |
| 112113 | Ph | Ph | CN | H | H |
| 112114 | Ph | Ph | DPA | H | H |
| 112115 | Ph | Ph | 2- | H | H |
| | | | DBF | | |
| 112116 | Ph | Ph | 2- | H | H |
| | | | DBT | | |
| 112117 | Ph | Ph | 4-t- | H | H |
| | | | BuPh | | |
| 112118 | Ph | Ph | 1,3- | H | H |
| | | | di-t- | | |
| | | | BuPh | | |
| 112119 | Ph | Ph | 9- | H | H |
| | | | Cbz | | |
| 112120 | Ph | Ph | t-Bu | t-Bu | t-Bu |
| 112121 | Ph | Ph | Ph | t-Bu | t-Bu |
| 112122 | Ph | Ph | CN | t-Bu | t-Bu |
| 112123 | Ph | Ph | DPA | t-Bu | t-Bu |
| 112124 | Ph | Ph | 2- | t-Bu | t-Bu |
| | | | DBF | | |
| 112125 | Ph | Ph | 2- | t-Bu | t-Bu |
| | | | DBT | | |
| 112126 | Ph | Ph | 4-t- | t-Bu | t-Bu |
| | | | BuPh | | |
| 112127 | Ph | Ph | 1,3- | t-Bu | t-Bu |
| | | | di-t- | | |
| | | | BuPh | | |
| 112128 | Ph | Ph | 9- | t-Bu | t-Bu |
| | | | Cbz | | |
| 112129 | Ph | Ph | Ph | Ph | Ph |
| 112130 | Ph | Ph | CN | Ph | Ph |
| 112131 | Ph | Ph | DPA | Ph | Ph |
| 112132 | Ph | Ph | 2- | Ph | Ph |
| | | | DBF | | |
| 112133 | Ph | Ph | 2- | Ph | Ph |
| | | | DBT | | |
| 112134 | Ph | Ph | 4-t- | Ph | Ph |
| | | | BuPh | | |
| 112135 | Ph | Ph | 1,3- | Ph | Ph |
| | | | di-t- | | |
| | | | BuPh | | |
| 112136 | Ph | Ph | 9- | Ph | Ph |
| | | | Cbz | | |
| 112137 | Ph | Ph | t-Bu | CN | CN |
| 112138 | Ph | Ph | Ph | CN | CN |
| 112139 | Ph | Ph | CN | CN | CN |
| 112140 | Ph | Ph | DPA | CN | CN |
| 112141 | Ph | Ph | 2- | CN | CN |
| | | | DBF | | |
| 112142 | Ph | Ph | 2- | CN | CN |
| | | | DBT | | |
| 112143 | Ph | Ph | 4-t- | CN | CN |
| | | | BuPh | | |
| 112144 | Ph | Ph | 1,3- | CN | CN |
| | | | di-t- | | |
| | | | BuPh | | |
| 112145 | Ph | Ph | 9- | CN | CN |
| | | | Cbz | | |
| 112146 | Ph | Ph | t-Bu | 9- | 9- |
| | | | | Cbz | Cbz |
| 112147 | Ph | Ph | Ph | 9- | 9- |
| | | | | Cbz | Cbz |
| 112148 | Ph | Ph | CN | 9- | 9- |
| | | | | Cbz | Cbz |
| 112149 | Ph | Ph | DPA | 9- | 9- |
| | | | | Cbz | Cbz |
| 112150 | Ph | Ph | 2- | 9- | 9- |
| | | | DBF | Cbz | Cbz |
| 112151 | Ph | Ph | 2- | 9- | 9- |
| | | | DBT | Cbz | Cbz |
| 112152 | Ph | Ph | 4-t- | 9- | 9- |
| | | | BuPh | Cbz | Cbz |
| 112153 | Ph | Ph | 1,3- | 9- | 9- |
| | | | di-t- | Cbz | Cbz |
| | | | BuPh | | |
| 112154 | Ph | Ph | 9- | 9- | 9- |
| | | | Cbz | Cbz | Cbz |
| 112155 | Ph | Ph | t-Bu | 4-t- | 4-t- |
| | | | | BuPh | BuPh |
| 112156 | Ph | Ph | Ph | 4-t- | 4-t- |
| | | | | BuPh | BuPh |
| 112157 | Ph | Ph | CN | 4-t- | 4-t- |
| | | | | BuPh | BuPh |
| 112158 | Ph | Ph | DPA | 4-t- | 4-t- |
| | | | | BuPh | BuPh |
| 112159 | Ph | Ph | 2- | 4-t- | 4-t- |
| | | | DBF | BuPh | BuPh |
| 112160 | Ph | Ph | 2- | 4-t- | 4-t- |
| | | | DBT | BuPh | BuPh |
| 112161 | Ph | Ph | 4-t- | 4-t- | 4-t- |
| | | | BuPh | BuPh | BuPh |
| 112162 | Ph | Ph | 1,3- | 4-t- | 4-t- |
| | | | di-t- | BuPh | BuPh |
| | | | BuPh | | |
| 112163 | Ph | Ph | 9- | 4-t- | 4-t- |
| | | | Cbz | BuPh | BuPh |
| 112164 | Ph | Ph | t-Bu | 1,3- | 1,3- |
| | | | | di-t- | di-t- |
| | | | | BuPh | BuPh |
| 112165 | Ph | Ph | Ph | 1,3- | 1,3- |
| | | | | di-t- | di-t- |
| | | | | BuPh | BuPh |
| 112166 | Ph | Ph | CN | 1,3- | 1,3- |
| | | | | di-t- | di-t- |
| | | | | BuPh | BuPh |
| 112167 | Ph | Ph | DPA | 1,3- | 1,3- |
| | | | | di-t- | di-t- |
| | | | | BuPh | BuPh |
| 112168 | Ph | Ph | 2- | 1,3- | 1,3- |
| | | | DBF | di-t- | di-t- |
| | | | | BuPh | BuPh |
| 112169 | Ph | Ph | 2- | 1,3- | 1,3- |
| | | | DBT | di-t- | di-t- |
| | | | | BuPh | BuPh |
| 112170 | Ph | Ph | 4-t- | 1,3- | 1,3- |
| | | | BuPh | di-t- | di-t- |
| | | | | BuPh | BuPh |
| 112171 | Ph | Ph | 1,3- | 1,3- | 1,3- |
| | | | di-t- | di-t- | di-t- |
| | | | BuPh | BuPh | BuPh |
| 112172 | Ph | Ph | 9- | 1,3- | 1,3- |
| | | | Cbz | di-t- | di-t- |
| | | | | BuPh | BuPh |
| 112173 | 9-Cbz | 9-Cbz | t-Bu | H | H |
| 112174 | 9-Cbz | 9-Cbz | Ph | H | H |
| 112175 | 9-Cbz | 9-Cbz | CN | H | H |
| 112176 | 9-Cbz | 9-Cbz | DPA | H | H |
| 112177 | 9-Cbz | 9-Cbz | 2- | H | H |
| | | | DBF | | |
| 112178 | 9-Cbz | 9-Cbz | 2- | H | H |
| | | | DBT | | |
| 112179 | 9-Cbz | 9-Cbz | 4-t- | H | H |
| | | | BuPh | | |
| 112180 | 9-Cbz | 9-Cbz | 1,3- | H | H |
| | | | di-t- | | |
| | | | BuPh | | |
| 112181 | 9-Cbz | 9-Cbz | t-Bu | t-Bu | t-Bu |
| 112182 | 9-Cbz | 9-Cbz | Ph | t-Bu | t-Bu |
| 112183 | 9-Cbz | 9-Cbz | CN | t-Bu | t-Bu |
| 112184 | 9-Cbz | 9-Cbz | DPA | t-Bu | t-Bu |
| 112185 | 9-Cbz | 9-Cbz | 2- | t-Bu | t-Bu |
| | | | DBF | | |
| 112186 | 9-Cbz | 9-Cbz | 2- | t-Bu | t-Bu |
| | | | DBT | | |
| 112187 | 9-Cbz | 9-Cbz | 4-t- | t-Bu | t-Bu |
| | | | BuPh | | |
| 112188 | 9-Cbz | 9-Cbz | 1,3- | t-Bu | t-Bu |
| | | | di-t- | | |
| | | | BuPh | | |
| 112189 | 9-Cbz | 9-Cbz | 9- | t-Bu | t-Bu |
| | | | Cbz | | |
| 112190 | 9-Cbz | 9-Cbz | t-Bu | Ph | Ph |
| 112191 | 9-Cbz | 9-Cbz | Ph | Ph | Ph |
| 112192 | 9-Cbz | 9-Cbz | CN | Ph | Ph |
| 112193 | 9-Cbz | 9-Cbz | DPA | Ph | Ph |
| 112194 | 9-Cbz | 9-Cbz | 2- | Ph | Ph |
| | | | DBF | | |
| 112195 | 9-Cbz | 9-Cbz | 2- | Ph | Ph |
| | | | DBT | | |
| 112196 | 9-Cbz | 9-Cbz | 4-t- | Ph | Ph |
| | | | BuPh | | |
| 112197 | 9-Cbz | 9-Cbz | 1,3- | Ph | Ph |
| | | | di-t- | | |
| | | | BuPh | | |
| 112198 | 9-Cbz | 9-Cbz | 9- | Ph | Ph |
| | | | Cbz | | |
| 112199 | 9-Cbz | 9-Cbz | t-Bu | CN | CN |
| 112200 | 9-Cbz | 9-Cbz | Ph | CN | CN |
| 112201 | 9-Cbz | 9-Cbz | CN | CN | CN |
| 112202 | 9-Cbz | 9-Cbz | DPA | CN | CN |
| 112203 | 9-Cbz | 9-Cbz | 2- | CN | CN |
| | | | DBF | | |
| 112204 | 9-Cbz | 9-Cbz | 2- | CN | CN |
| | | | DBT | | |
| 112205 | 9-Cbz | 9-Cbz | 4-t- | CN | CN |
| | | | BuPh | | |
| 112206 | 9-Cbz | 9-Cbz | 1,3- | CN | CN |
| | | | di-t- | | |
| | | | BuPh | | |
| 112207 | 9-Cbz | 9-Cbz | 9- | CN | CN |
| | | | Cbz | | |
| 112208 | 9-Cbz | 9-Cbz | t-Bu | 9- | 9- |
| | | | | Cbz | Cbz |
| 112209 | 9-Cbz | 9-Cbz | Ph | 9- | 9- |
| | | | | Cbz | Cbz |
| 112210 | 9-Cbz | 9-Cbz | CN | 9- | 9- |
| | | | | Cbz | Cbz |
| 112211 | 9-Cbz | 9-Cbz | DPA | 9-Cbz | 9-Cbz |
| 112212 | 9-Cbz | 9-Cbz | 2- | 9-Cbz | 9-Cbz |
| | | | DBF | | |
| 112213 | 9-Cbz | 9-Cbz | 2- | 9-Cbz | 9-Cbz |
| | | | DBT | | |
| 112214 | 9-Cbz | 9-Cbz | 4-t- | 9-Cbz | 9-Cbz |
| | | | BuPh | | |
| 112215 | 9-Cbz | 9-Cbz | 1,3- | 9-Cbz | 9-Cbz |
| | | | di-t- | | |
| | | | BuPh | | |
| 112216 | 9-Cbz | 9-Cbz | 9-Cbz | 9-Cbz | 9-Cbz |
| 112217 | 9-Cbz | 9-Cbz | t-Bu | 4-t- | 4-t- |
| | | | | BuPh | BuPh |
| 112218 | 9-Cbz | 9-Cbz | Ph | 4-t- | 4-t- |
| | | | | BuPh | BuPh |
| 112219 | 9-Cbz | 9-Cbz | CN | 4-t- | 4-t- |
| | | | | BuPh | BuPh |
| 112220 | 9-Cbz | 9-Cbz | DPA | 4-t- | 4-t- |
| | | | | BuPh | BuPh |
| 112221 | 9-Cbz | 9-Cbz | 2- | 4-t- | 4-t- |
| | | | DBF | BuPh | BuPh |
| 112222 | 9-Cbz | 9-Cbz | 2- | 4-t- | 4-t- |
| | | | DBT | BuPh | BuPh |
| 112223 | 9-Cbz | 9-Cbz | 4-t- | 4-t- | 4-t- |
| | | | BuPh | BuPh | BuPh |
| 112224 | 9-Cbz | 9-Cbz | 1,3- | 4-t- | 4-t- |
| | | | di-t- | BuPh | BuPh |
| | | | BuPh | | |
| 112225 | 9-Cbz | 9-Cbz | 9-Cbz | 4-t- | 4-t- |
| | | | | BuPh | BuPh |
| 112226 | 9-Cbz | 9-Cbz | t-Bu | 1,3- | 1,3- |
| | | | | di-t- | di-t- |
| | | | | BuPh | BuPh |
| 112227 | 9-Cbz | 9-Cbz | Ph | 1,3- | 1,3- |
| | | | | di-t- | di-t- |
| | | | | BuPh | BuPh |
| 112228 | 9-Cbz | 9-Cbz | CN | 1,3- | 1,3- |
| | | | | di-t- | di-t- |
| | | | | BuPh | BuPh |
| 112229 | 9-Cbz | 9-Cbz | DPA | 1,3- | 1,3- |
| | | | | di-t- | di-t- |
| | | | | BuPh | BuPh |
| 112230 | 9-Cbz | 9-Cbz | 2- | 1,3- | 1,3- |
| | | | DBF | di-t- | di-t- |
| | | | | BuPh | BuPh |
| 112231 | 9-Cbz | 9-Cbz | 2- | 1,3- | 1,3- |
| | | | DBT | di-t- | di-t- |
| | | | | BuPh | BuPh |
| 112232 | 9-Cbz | 9-Cbz | 4-t- | 1,3- | 1,3- |
| | | | BuPh | di-t- | di-t- |
| | | | | BuPh | BuPh |
| 112233 | 9-Cbz | 9-Cbz | 1,3- | 1,3- | 1,3- |
| | | | di-t- | di-t- | di-t- |
| | | | BuPh | BuPh | BuPh |
| 112234 | 9-Cbz | 9-Cbz | 9-Cbz | 1,3- | 1,3- |
| | | | | di-t- | di-t- |
| | | | | BuPh | BuPh |
| 112235 | CN | CN | t-Bu | H | H |
| 112236 | CN | CN | Ph | H | H |
| 112237 | CN | CN | DPA | H | H |
| 112238 | CN | CN | 2- | H | H |
| | | | DBF | | |
| 112239 | CN | CN | 2- | H | H |
| | | | DBT | | |
| 112240 | CN | CN | 4-t- | H | H |
| | | | BuPh | | |
| 112241 | CN | CN | 1,3- | H | H |
| | | | di-t- | | |
| | | | BuPh | | |
| 112242 | CN | CN | 9-Cbz | H | H |
| 112243 | CN | CN | t-Bu | t-Bu | t-Bu |
| 112244 | CN | CN | Ph | t-Bu | t-Bu |
| 112245 | CN | CN | CN | t-Bu | t-Bu |
| 112246 | CN | CN | DPA | t-Bu | t-Bu |
| 112247 | CN | CN | 2- | t-Bu | t-Bu |
| | | | DBF | | |
| 112248 | CN | CN | 2- | t-Bu | t-Bu |
| | | | DBT | | |
| 112249 | CN | CN | 4-t- | t-Bu | t-Bu |
| | | | BuPh | | |
| 112250 | CN | CN | 1,3- | t-Bu | t-Bu |
| | | | di-t- | | |
| | | | BuPh | | |
| 112251 | CN | CN | 9-Cbz | t-Bu | t-Bu |
| 112252 | CN | CN | t-Bu | Ph | Ph |
| 112253 | CN | CN | Ph | Ph | Ph |
| 112254 | CN | CN | CN | Ph | Ph |
| 112255 | CN | CN | DPA | Ph | Ph |
| 112256 | CN | CN | 2- | Ph | Ph |
| | | | DBF | | |
| 112257 | CN | CN | 2- | Ph | Ph |
| | | | DBT | | |
| 112258 | CN | CN | 4-t- | Ph | Ph |
| | | | BuPh | | |
| 112259 | CN | CN | 1,3- | Ph | Ph |
| | | | di-t- | | |
| | | | BuPh | | |
| 112260 | CN | CN | 9-Cbz | Ph | Ph |
| 112261 | CN | CN | t-Bu | CN | CN |
| 112262 | CN | CN | Ph | CN | CN |
| 112263 | CN | CN | CN | CN | CN |
| 112264 | CN | CN | DPA | CN | CN |
| 112265 | CN | CN | 2- | CN | CN |
| | | | DBF | | |
| 112266 | CN | CN | 2- | CN | CN |
| | | | DBT | | |
| 112267 | CN | CN | 4-t- | CN | CN |
| | | | BuPh | | |
| 112268 | CN | CN | 1,3- | CN | CN |
| | | | di-t- | | |
| | | | BuPh | | |
| 112269 | CN | CN | 9-Cbz | CN | CN |
| 112270 | CN | CN | t-Bu | 9-Cbz | 9-Cbz |
| 112271 | CN | CN | Ph | 9-Cbz | 9-Cbz |
| 112272 | CN | CN | CN | 9-Cbz | 9-Cbz |
| 112273 | CN | CN | DPA | 9-Cbz | 9-Cbz |
| 112274 | CN | CN | 2- | 9-Cbz | 9-Cbz |
| | | | DBF | | |
| 112275 | CN | CN | 2- | 9-Cbz | 9-Cbz |
| | | | DBT | | |
| 112276 | CN | CN | 4-t- | 9-Cbz | 9-Cbz |
| | | | BuPh | | |
| 112277 | CN | CN | 1,3- | 9-Cbz | 9-Cbz |
| | | | di-t- | | |
| | | | BuPh | | |
| 112278 | CN | CN | 9-Cbz | 9-Cbz | 9-Cbz |
| 112279 | CN | CN | t-Bu | 4-t- | 4-t- |
| | | | | BuPh | BuPh |
| 112280 | CN | CN | Ph | 4-t- | 4-t- |
| | | | | BuPh | BuPh |
| 112281 | CN | CN | CN | 4-t- | 4-t- |
| | | | | BuPh | BuPh |
| 112282 | CN | CN | DPA | 4-t- | 4-t- |
| | | | | BuPh | BuPh |
| 112283 | CN | CN | 2- | 4-t- | 4-t- |
| | | | DBF | BuPh | BuPh |
| 112284 | CN | CN | 2- | 4-t- | 4-t- |
| | | | DBT | BuPh | BuPh |
| 112285 | CN | CN | 4-t- | 4-t- | 4-t- |
| | | | BuPh | BuPh | BuPh |
| 112286 | CN | CN | 1,3- | 4-t- | 4-t- |
| | | | di-t- | BuPh | BuPh |
| | | | BuPh | | |
| 112287 | CN | CN | 9-Cbz | 4-t- | 4-t- |
| | | | | BuPh | BuPh |
| 112288 | CN | CN | t-Bu | 1,3- | 1,3- |
| | | | | di-t- | di-t- |
| | | | | BuPh | BuPh |
| 112289 | CN | CN | Ph | 1,3- | 1,3- |
| | | | | di-t- | di-t- |
| | | | | BuPh | BuPh |
| 112290 | CN | CN | CN | 1,3- | 1.3- |
| | | | | di-t- | di-t- |
| | | | | BuPh | BuPh |
| 112291 | CN | CN | DPA | 1,3- | 1,3- |
| | | | | di-t- | di-t- |
| | | | | BuPh | BuPh |
| 112292 | CN | CN | 2- | 1.3- | 1.3- |
| | | | DBF | di-t- | di-t- |
| | | | | BuPh | BuPh |
| 112293 | CN | CN | 2- | 1,3- | 1.3- |
| | | | DBT | di-t- | di-t- |
| | | | | BuPh | BuPh |
| 112294 | CN | CN | 4-t- | 1,3- | 1,3- |
| | | | BuPh | di-t- | di-t- |
| | | | | BuPh | BuPh |
| 112295 | CN | CN | 1,3- | 1,3- | 1,3- |
| | | | di-t- | di-t- | di-t- |
| | | | BuPh | BuPh | BuPh |
| 112296 | CN | CN | 9-Cbz | 1,3- | 1,3- |
| | | | | di-t- | di-t- |
| | | | | BuPh | BuPh |
| 112297 | 4-t- | 4-t- | t-Bu | H | H |
| | BuPh | BuPh | | | |
| 112298 | 4-t- | 4-t- | Ph | H | H |
| | BuPh | BuPh | | | |
| 112299 | 4-t- | 4-t- | CN | H | H |
| | BuPh | BuPh | | | |
| 112300 | 4-t- | 4-t- | DPA | H | H |
| | BuPh | BuPh | | | |
| 112301 | 4-t- | 4-t- | 2- | H | H |
| | BuPh | BuPh | DBF | | |
| 112302 | 4-t- | 4-t- | 2- | H | H |
| | BuPh | BuPh | DBT | | |
| 112303 | 4-t- | 4-t- | 1,3- | H | H |
| | BuPh | BuPh | di-t- | | |
| | | | BuPh | | |
| 112304 | 4-t- | 4-t- | 9-Cbz | H | H |
| | BuPh | BuPh | | | |
| 112305 | 4-t- | 4-t- | t-Bu | t-Bu | t-Bu |
| | BuPh | BuPh | | | |
| 112306 | 4-t- | 4-t- | Ph | t-Bu | t-Bu |
| | BuPh | BuPh | | | |
| 112307 | 4-t- | 4-t- | CN | t-Bu | t-Bu |
| | BuPh | BuPh | | | |
| 112308 | 4-t- | 4-t- | DPA | t-Bu | t-Bu |
| | BuPh | BuPh | | | |
| 112309 | 4-t- | 4-t- | 2- | t-Bu | t-Bu |
| | BuPh | BuPh | DBF | | |
| 112310 | 4-t- | 4-t- | 2- | t-Bu | t-Bu |
| | BuPh | BuPh | DBT | | |
| 112311 | 4-t- | 4-t- | 4-t- | t-Bu | t-Bu |
| | BuPh | BuPh | BuPh | | |
| 112312 | 4-t- | 4-t- | 1,3- | t-Bu | t-Bu |
| | BuPh | BuPh | di-t- | | |
| | | | BuPh | | |
| 112313 | 4-t- | 4-t- | 9-Cbz | t-Bu | t-Bu |
| | BuPh | BuPh | | | |
| 112314 | 4-t- | 4-t- | t-Bu | Ph | Ph |
| | BuPh | BuPh | | | |
| 112315 | 4-t- | 4-t- | Ph | Ph | Ph |
| | BuPh | BuPh | | | |
| 112316 | 4-t- | 4-t- | CN | Ph | Ph |
| | BuPh | BuPh | | | |
| 112317 | 4-t- | 4-t- | DPA | Ph | Ph |
| | BuPh | BuPh | | | |
| 112318 | 4-t- | 4-t- | 2- | Ph | Ph |
| | BuPh | BuPh | DBF | | |
| 112319 | 4-t- | 4-t- | 2- | Ph | Ph |
| | BuPh | BuPh | DBT | | |
| 112320 | 4-t- | 4-t- | 4-t- | Ph | Ph |
| | BuPh | BuPh | BuPh | | |
| 112321 | 4-t- | 4-t- | 1,3- | Ph | Ph |
| | BuPh | BuPh | di-t- | | |
| | | | BuPh | | |
| 112322 | 4-t- | 4-t- | 9-Cbz | Ph | Ph |
| | BuPh | BuPh | | | |
| 112323 | 4-t- | 4-t- | t-Bu | CN | CN |
| | BuPh | BuPh | | | |
| 112324 | 4-t- | 4-t- | Ph | CN | CN |
| | BuPh | BuPh | | | |
| 112325 | 4-t- | 4-t- | CN | CN | CN |
| | BuPh | BuPh | | | |
| 112326 | 4-t- | 4-t- | DPA | CN | CN |
| | BuPh | BuPh | | | |
| 112327 | 4-t- | 4-t- | 2- | CN | CN |
| | BuPh | BuPh | DBF | | |
| 112328 | 4-t- | 4-t- | 2- | CN | CN |
| | BuPh | BuPh | DBT | | |
| 112329 | 4-t- | 4-t- | 4-t- | CN | CN |
| | BuPh | BuPh | BuPh | | |
| 112330 | 4-t- | 4-t- | 1,3- | CN | CN |
| | BuPh | BuPh | di-t- | | |
| | | | BuPh | | |
| 112331 | 4-t- | 4-t- | 9-Cbz | CN | CN |
| | BuPh | BuPh | | | |
| 112332 | 4-t- | 4-t- | t-Bu | 9-Cbz | 9-Cbz |
| | BuPh | BuPh | | | |
| 112333 | 4-t- | 4-t- | Ph | 9-Cbz | 9-Cbz |
| | BuPh | BuPh | | | |
| 112334 | 4-t- | 4-t- | CN | 9-Cbz | 9-Cbz |
| | BuPh | BuPh | | | |
| 112335 | 4-t- | 4-t- | DPA | 9-Cbz | 9-Cbz |
| | BuPh | BuPh | | | |
| 112336 | 4-t- | 4-t- | 2- | 9-Cbz | 9-Cbz |
| | BuPh | BuPh | DBF | | |
| 112337 | 4-t- | 4-t- | 2- | 9-Cbz | 9-Cbz |
| | BuPh | BuPh | DBT | | |
| 112338 | 4-t- | 4-t- | 4-t- | 9-Cbz | 9-Cbz |
| | BuPh | BuPh | BuPh | | |
| 112339 | 4-t- | 4-t- | 1,3- | 9-Cbz | 9-Cbz |
| | BuPh | BuPh | di-t- | | |
| | | | BuPh | | |
| 112340 | 4-t- | 4-t- | 9-Cbz | 9-Cbz | 9-Cbz |
| | BuPh | BuPh | | | |
| 112341 | 4-t- | 4-t- | t-Bu | 4-t- | 4-t- |
| | BuPh | BuPh | | BuPh | BuPh |
| 112342 | 4-t- | 4-t- | Ph | 4-t- | 4-t- |
| | BuPh | BuPh | | BuPh | BuPh |
| 112343 | 4-t- | 4-t- | CN | 4-t- | 4-t- |
| | BuPh | BuPh | | BuPh | BuPh |
| 112344 | 4-t- | 4-t- | DPA | 4-t- | 4-t- |
| | BuPh | BuPh | | BuPh | BuPh |
| 112345 | 4-t- | 4-t- | 2- | 4-t- | 4-t- |
| | BuPh | BuPh | DBF | BuPh | BuPh |
| 112346 | 4-t- | 4-t- | 2- | 4-t- | 4-t- |
| | BuPh | BuPh | DBT | BuPh | BuPh |
| 112347 | 4-t- | 4-t- | 4-t- | 4-t- | 4-t- |
| | BuPh | BuPh | BuPh | BuPh | BuPh |
| 112348 | 4-t- | 4-t- | 1,3- | 4-t- | 4-t- |
| | BuPh | BuPh | di-t- | BuPh | BuPh |
| | | | BuPh | | |
| 112349 | 4-t- | 4-t- | 9-Cbz | 4-t- | 4-t- |
| | BuPh | BuPh | | BuPh | BuPh |
| 112350 | 4-t- | 4-t- | t-Bu | 1,3- | 1,3- |
| | BuPh | BuPh | | di-t- | di-t- |
| | | | | BuPh | BuPh |
| 112351 | 4-t- | 4-t- | Ph | 1,3- | 1,3- |
| | BuPh | BuPh | | di-t- | di-t- |
| | | | | BuPh | BuPh |
| 112352 | 4-t- | 4-t- | CN | 1,3- | 1,3- |
| | BuPh | BuPh | | di-t- | di-t- |
| | | | | BuPh | BuPh |
| 112353 | 4-t- | 4-t- | DPA | 1,3- | 1,3- |
| | BuPh | BuPh | | di-t- | di-t- |
| | | | | BuPh | BuPh |
| 112354 | 4-t- | 4-t- | 2- | 1,3- | 1,3- |
| | BuPh | BuPh | DBF | di-t- | di-t- |
| | | | | BuPh | BuPh |
| 112355 | 4-t- | 4-t- | 2- | 1,3- | 1,3- |
| | BuPh | BuPh | DBT | di-t- | di-t- |
| | | | | BuPh | BuPh |
| 112356 | 4-t- | 4-t- | 4-t- | 1,3- | 1,3- |
| | BuPh | BuPh | BuPh | di-t- | di-t- |
| | | | | BuPh | BuPh |
| 112357 | 4-t- | 4-t- | 1,3- | 1,3- | 1,3- |
| | BuPh | BuPh | di-t- | di-t- | di-t- |
| | | | BuPh | BuPh | BuPh |
| 112358 | 4-t- | 4-t- | 9-Cbz | 1,3- | 1,3- |
| | BuPh | BuPh | | di-t- | di-t- |
| | | | | BuPh | BuPh |
| 112359 | 1,3- | 1,3- | t-Bu | H | H |
| | di-t- | di-t- | | | |
| | BuPh | BuPh | | | |
| 112360 | 1,3- | 1,3- | Ph | H | H |
| | di-t- | di-t- | | | |
| | BuPh | BuPh | | | |
| 112361 | 1,3- | 1,3- | CN | H | H |
| | di-t- | di-t- | | | |
| | BuPh | BuPh | | | |
| 112362 | 1,3- | 1,3- | DPA | H | H |
| | di-t- | di-t- | | | |
| | BuPh | BuPh | | | |
| 112363 | 1,3- | 1,3- | 2- | H | H |
| | di-t- | di-t- | DBF | | |
| | BuPh | BuPh | | | |
| 112364 | 1,3- | 1,3- | 2- | H | H |
| | di-t- | di-t- | DBT | | |
| | BuPh | BuPh | | | |
| 112365 | 1,3- | 1,3- | 4-t- | H | H |
| | di-t- | di-t- | BuPh | | |
| | BuPh | BuPh | | | |
| 112366 | 1,3- | 1,3- | 9-Cbz | H | H |
| | di-t- | di-t- | | | |
| | BuPh | BuPh | | | |
| 112367 | 1,3- | 1,3- | t-Bu | t-Bu | t-Bu |
| | di-t- | di-t- | | | |
| | BuPh | BuPh | | | |
| 112368 | 1,3- | 1,3- | Ph | t-Bu | t-Bu |
| | di-t- | di-t- | | | |
| | BuPh | BuPh | | | |
| 112369 | 1,3- | 1,3- | CN | t-Bu | t-Bu |
| | di-t- | di-t- | | | |
| | BuPh | BuPh | | | |
| 112370 | 1,3- | 1,3- | DPA | t-Bu | t-Bu |
| | di-t- | di-t- | | | |
| | BuPh | BuPh | | | |
| 112371 | 1,3- | 1,3- | 2- | t-Bu | t-Bu |
| | di-t- | di-t- | DBF | | |
| | BuPh | BuPh | | | |
| 112372 | 1,3- | 1,3- | 2- | t-Bu | t-Bu |
| | di-t- | di-t- | DBT | | |
| | BuPh | BuPh | | | |
| 112373 | 1,3- | 1,3- | 4-t- | t-Bu | t-Bu |
| | di-t- | di-t- | BuPh | | |
| | BuPh | BuPh | | | |
| 112374 | 1,3- | 1,3- | 1,3- | t-Bu | t-Bu |
| | di-t- | di-t- | di-t- | | |
| | BuPh | BuPh | BuPh | | |
| 112375 | 1,3- | 1,3- | 9-Cbz | t-Bu | t-Bu |
| | di-t- | di-t- | | | |
| | BuPh | BuPh | | | |
| 112376 | 1,3- | 1,3- | t-Bu | Ph | Ph |
| | di-t- | di-t- | | | |
| | BuPh | BuPh | | | |
| 112377 | 1,3- | 1,3- | Ph | Ph | Ph |
| | di-t- | di-t- | | | |
| | BuPh | BuPh | | | |
| 112378 | 1,3- | 1,3- | CN | Ph | Ph |
| | di-t- | di-t- | | | |
| | BuPh | BuPh | | | |
| 112379 | 1,3- | 1,3- | DPA | Ph | Ph |
| | di-t- | di-t- | | | |
| | BuPh | BuPh | | | |
| 112380 | 1,3- | 1,3- | 2- | Ph | Ph |
| | di-t- | di-t- | DBF | | |
| | BuPh | BuPh | | | |
| 112381 | 1,3- | 1,3- | 2- | Ph | Ph |
| | di-t- | di-t- | DBT | | |
| | BuPh | BuPh | | | |
| 112382 | 1,3- | 1,3- | 4-t- | Ph | Ph |
| | di-t- | di-t- | BuPh | | |
| | BuPh | BuPh | | | |
| 112383 | 1,3- | 1,3- | 1,3- | Ph | Ph |
| | di-t- | di-t- | di-t- | | |
| | BuPh | BuPh | BuPh | | |
| 112384 | 1,3- | 1,3- | 9-Cbz | Ph | Ph |
| | di-t- | di-t- | | | |
| | BuPh | BuPh | | | |
| 112385 | 1,3- | 1,3- | t-Bu | CN | CN |
| | di-t- | di-t- | | | |
| | BuPh | BuPh | | | |
| 112386 | 1,3- | 1,3- | Ph | CN | CN |
| | di-t- | di-t- | | | |
| | BuPh | BuPh | | | |
| 112387 | 1,3- | 1,3- | CN | CN | CN |
| | di-t- | di-t- | | | |
| | BuPh | BuPh | | | |
| 112388 | 1,3- | 1,3- | DPA | CN | CN |
| | di-t- | di-t- | | | |
| | BuPh | BuPh | | | |
| 112389 | 1,3- | 1,3- | 2- | CN | CN |
| | di-t- | di-t- | DBF | | |
| | BuPh | BuPh | | | |
| 112390 | 1,3- | 1,3- | 2- | CN | CN |
| | di-t- | di-t- | DBT | | |
| | BuPh | BuPh | | | |
| 112391 | 1,3- | 1,3- | 4-t- | CN | CN |
| | di-t- | di-t- | BuPh | | |
| | BuPh | BuPh | | | |
| 112392 | 1,3- | 1,3- | 1,3- | CN | CN |
| | di-t- | di-t- | di-t- | | |
| | BuPh | BuPh | BuPh | | |
| 112393 | 1,3- | 1,3- | 9-Cbz | CN | CN |
| | di-t- | di-t- | | | |
| | BuPh | BuPh | | | |
| 112394 | 1,3- | 1,3- | t-Bu | 9-Cbz | 9-Cbz |
| | di-t- | di-t- | | | |
| | BuPh | BuPh | | | |
| 112395 | 1,3- | 1,3- | Ph | 9-Cbz | 9-Cbz |
| | di-t- | di-t- | | | |
| | BuPh | BuPh | | | |
| 112396 | 1,3- | 1,3- | CN | 9-Cbz | 9-Cbz |
| | di-t- | di-t- | | | |
| | BuPh | BuPh | | | |
| 112397 | 1,3- | 1,3- | DPA | 9-Cbz | 9-Cbz |
| | di-t- | di-t- | | | |
| | BuPh | BuPh | | | |
| 112398 | 1,3- | 1,3- | 2- | 9-Cbz | 9-Cbz |
| | di-t- | di-t- | DBF | | |
| | BuPh | BuPh | | | |
| 112399 | 1,3- | 1,3- | 2- | 9-Cbz | 9-Cbz |
| | di-t- | di-t- | DBT | | |
| | BuPh | BuPh | | | |
| 112400 | 1,3- | 1,3- | 4-t- | 9-Cbz | 9-Cbz |
| | di-t- | di-t- | BuPh | | |
| | BuPh | BuPh | | | |
| 112401 | 1,3- | 1,3- | 1,3- | 9-Cbz | 9-Cbz |
| | di-t- | di-t- | di-t- | | |
| | BuPh | BuPh | BuPh | | |
| 112402 | 1,3- | 1,3- | 9-Cbz | 9-Cbz | 9-Cbz |
| | di-t- | di-t- | | | |
| | BuPh | BuPh | | | |
| 112403 | 1,3- | 1,3- | t-Bu | 4-t- | 4-t- |
| | di-t- | di-t- | | BuPh | BuPh |
| | BuPh | BuPh | | | |
| 112404 | 1,3- | 1,3- | Ph | 4-t- | 4-t- |
| | di-t- | di-t- | | BuPh | BuPh |
| | BuPh | BuPh | | | |
| 112405 | 1,3- | 1,3- | CN | 4-t- | 4-t- |
| | di-t- | di-t- | | BuPh | BuPh |
| | BuPh | BuPh | | | |
| 112406 | 1,3- | 1,3- | DPA | 4-t- | 4-t- |
| | di-t- | di-t- | | BuPh | BuPh |
| | BuPh | BuPh | | | |
| 112407 | 1,3- | 1,3- | 2- | 4-t- | 4-t- |
| | di-t- | di-t- | DBF | BuPh | BuPh |
| | BuPh | BuPh | | | |
| 112408 | 1,3- | 1,3- | 2- | 4-t- | 4-t- |
| | di-t- | di-t- | DBT | BuPh | BuPh |
| | BuPh | BuPh | | | |
| 112409 | 1,3- | 1,3- | 4-t- | 4-t- | 4-t- |
| | di-t- | di-t- | BuPh | BuPh | BuPh |
| | BuPh | BuPh | | | |
| 112410 | 1,3- | 1,3- | 1,3- | 4-t- | 4-t- |
| | di-t- | di-t- | di-t- | BuPh | BuPh |
| | BuPh | BuPh | BuPh | | |
| 112411 | 1,3- | 1,3- | 9-Cbz | 4-t- | 4-t- |
| | di-t- | di-t- | | BuPh | BuPh |
| | BuPh | BuPh | | | |
| 112412 | 1,3- | 1,3- | t-Bu | 1,3- | 1,3- |
| | di-t- | di-t- | | di-t- | di-t- |
| | BuPh | BuPh | | BuPh | BuPh |
| 112413 | 1,3- | 1,3- | Ph | 1,3- | 1,3- |
| | di-t- | di-t- | | di-t- | di-t- |
| | BuPh | BuPh | | BuPh | BuPh |
| 112414 | 1,3- | 1,3- | CN | 1,3- | 1,3- |
| | di-t- | di-t- | | di-t- | di-t- |
| | BuPh | BuPh | | BuPh | BuPh |
| 112415 | 1,3- | 1,3- | DPA | 1,3- | 1,3- |
| | di-t- | di-t- | | di-t- | di-t- |
| | BuPh | BuPh | | BuPh | BuPh |
| 112416 | 1,3- | 1,3- | 2- | 1,3- | 1,3- |
| | di-t- | di-t- | DBF | di-t- | di-t- |
| | BuPh | BuPh | | BuPh | BuPh |
| 112417 | 1,3- | 1,3- | 2- | 1,3- | 1,3- |
| | di-t- | di-t- | DBT | di-t- | di-t- |
| | BuPh | BuPh | | BuPh | BuPh |
| 112418 | 1,3- | 1,3- | 4-t- | 1,3- | 1,3- |
| | di-t- | di-t- | BuPh | di-t- | di-t- |
| | BuPh | BuPh | | BuPh | BuPh |
| 112419 | 1,3- | 1,3- | 1,3- | 1,3- | 1,3- |
| | di-t- | di-t- | di-t- | di-t- | di-t- |
| | BuPh | BuPh | BuPh | BuPh | BuPh |
| 112420 | 1,3- | 1,3- | 9-Cbz | 1,3- | 1,3- |
| | di-t- | di-t- | | di-t- | di-t- |
| | BuPh | BuPh | | BuPh | BuPh |

### Group HC2

The heterocyclic compound may be represented by Formula 11-5, and substituents may be defined as shown in Table 2.

TABLE 2

| Compound | | | | | |
|---|---|---|---|---|---|
| No. | R_{11b} | R_{12c} | R_{14b} | R_{18b} | R_{19b} |
| 115001 | t-Bu | t-Bu | t-Bu | t-Bu | t-Bu |
| 115002 | t-Bu | t-Bu | t-Bu | Ph | Ph |
| 115003 | t-Bu | t-Bu | t-Bu | 4-t- | 4-t- |
| | | | | BuPh | BuPh |
| 115004 | t-Bu | t-Bu | t-Bu | 9-Cbz | 9-Cbz |
| 115005 | t-Bu | t-Bu | Ph | t-Bu | t-Bu |
| 115006 | t-Bu | t-Bu | Ph | Ph | Ph |
| 115007 | t-Bu | t-Bu | 9-Cbz | t-Bu | t-Bu |
| 115008 | Ph | Ph | t-Bu | t-Bu | t-Bu |
| 115009 | Ph | Ph | t-Bu | Ph | Ph |
| 115010 | t-Bu | t-Bu | H | H | H |
| 115011 | H | H | t-Bu | H | H |
| 115012 | H | H | H | t-Bu | t-Bu |
| 115013 | t-Bu | t-Bu | t-Bu | H | H |
| 115014 | H | H | t-Bu | t-Bu | t-Bu |
| 115015 | t-Bu | t-Bu | H | t-Bu | t-Bu |
| 115016 | Ph | Ph | H | H | H |
| 115017 | H | H | Ph | H | H |
| 115018 | H | H | H | Ph | Ph |
| 115019 | Ph | Ph | Ph | H | H |
| 115020 | H | H | Ph | Ph | Ph |
| 115021 | Ph | Ph | H | Ph | Ph |
| 115022 | CN | CN | H | H | H |
| 115023 | H | H | CN | H | H |
| 115024 | H | H | H | CN | CN |
| 115025 | CN | CN | CN | H | H |
| 115026 | H | H | CN | CN | CN |
| 115027 | CN | CN | H | CN | CN |
| 115028 | 9-Cbz | 9-Cbz | H | H | H |
| 115029 | H | H | 9-Cbz | H | H |
| 115030 | H | H | H | 9-Cbz | 9-Cbz |
| 115031 | 9-Cbz | 9-Cbz | 9-Cbz | H | H |
| 115032 | H | H | 9-Cbz | 9-Cbz | 9-Cbz |
| 115033 | 9-Cbz | 9-Cbz | H | 9-Cbz | 9-Cbz |
| 115034 | Me | Me | H | H | H |
| 115035 | H | H | Me | H | H |
| 115036 | H | H | H | Me | Me |
| 115037 | Me | Me | Me | H | H |
| 115038 | H | H | Me | Me | Me |
| 115039 | Me | Me | H | Me | Me |
| 115040 | CD₃ | CD3 | H | H | H |
| 115041 | H | H | CD3 | H | H |
| 115042 | H | H | H | CD3 | CD3 |
| 115043 | CD3 | CD3 | CD3 | H | H |
| 115044 | H | H | CD3 | CD3 | CD3 |
| 115045 | CD3 | CD3 | H | CD3 | CD3 |
| 115046 | 4-t- | 4-t- | H | H | H |
| | BuPh | BuPh | | | |
| 115047 | H | H | 4-t- | H | H |
| | | | BuPh | | |
| 115048 | H | H | H | 4-t- | 4-t- |
| | | | | BuPh | BuPh |
| 115049 | 4-t- | 4-t- | 4-t- | H | H |
| | BuPh | BuPh | BuPh | | |
| 115050 | H | H | 4-t- | 4-t- | 4-t- |
| | | | BuPh | BuPh | BuPh |
| 115051 | 4-t- | 4-t- | H | 4-t- | 4-t- |
| | BuPh | BuPh | | BuPh | BuPh |
| 115052 | 1,3- | 1,3- | H | H | H |
| | di-t- | di-t- | | | |
| | BuPh | BuPh | | | |
| 115053 | H | H | 1,3- | H | H |
| | | | di-t- | | |
| | | | BuPh | | |
| 115054 | H | H | H | 1,3- | 1,3- |
| | | | | di-t- | di-t- |
| | | | | BuPh | BuPh |
| 115055 | 1,3- | 1,3- | 1,3- | H | H |
| | di-t- | di-t- | di-t- | | |
| | BuPh | BuPh | BuPh | | |
| 115056 | H | H | 1,3- | 1,3- | 1,3- |
| | | | di-t- | di-t- | di-t- |
| | | | BuPh | BuPh | BuPh |
| 115057 | 1,3- | 1,3- | H | 1,3- | 1,3- |
| | di-t- | di-t- | | di-t- | di-t- |
| | BuPh | BuPh | | BuPh | BuPh |
| 115058 | t-Bu | t-Bu | Ph | H | H |
| 115059 | t-Bu | t-Bu | CN | H | H |
| 115060 | t-Bu | t-Bu | DPA | H | H |
| 115061 | t-Bu | t-Bu | 2- | H | H |
| | | | DBF | | |
| 115062 | t-Bu | t-Bu | 2- | H | H |
| | | | DBT | | |
| 115063 | t-Bu | t-Bu | 4-t- | H | H |
| | | | BuPh | | |
| 115064 | t-Bu | t-Bu | 1,3- | H | H |
| | | | di-t- | | |
| | | | BuPh | | |
| 115065 | t-Bu | t-Bu | 9-Cbz | H | H |
| 115066 | t-Bu | t-Bu | CN | t-Bu | t-Bu |
| 115067 | t-Bu | t-Bu | DPA | t-Bu | t-Bu |
| 115068 | t-Bu | t-Bu | 2- | t-Bu | t-Bu |
| | | | DBF | | |
| 115069 | t-Bu | t-Bu | 2- | t-Bu | t-Bu |
| | | | DBT | | |
| 115070 | t-Bu | t-Bu | 4-t- | t-Bu | t-Bu |
| | | | BuPh | | |
| 115071 | t-Bu | t-Bu | 1,3- | t-Bu | t-Bu |
| | | | di-t- | | |
| | | | BuPh | | |
| 115072 | t-Bu | t-Bu | CN | Ph | Ph |
| 115073 | t-Bu | t-Bu | DPA | Ph | Ph |
| 115074 | t-Bu | t-Bu | 2- | Ph | Ph |
| | | | DBF | | |
| 115075 | t-Bu | t-Bu | 2- | Ph | Ph |
| | | | DBT | | |
| 115076 | t-Bu | t-Bu | 4-t- | Ph | Ph |
| | | | BuPh | | |
| 115077 | t-Bu | t-Bu | 1,3- | Ph | Ph |
| | | | di-t- | | |
| | | | BuPh | | |
| 115078 | t-Bu | t-Bu | 9-Cbz | Ph | Ph |
| 115079 | t-Bu | t-Bu | t-Bu | CN | CN |
| 115080 | t-Bu | t-Bu | Ph | CN | CN |
| 115081 | t-Bu | t-Bu | CN | CN | CN |
| 115082 | t-Bu | t-Bu | DPA | CN | CN |
| 115083 | t-Bu | t-Bu | 2- | CN | CN |
| | | | DBF | | |
| 115084 | t-Bu | t-Bu | 2- | CN | CN |
| | | | DBT | | |
| 115085 | t-Bu | t-Bu | 4-t- | CN | CN |
| | | | BuPh | | |
| 115086 | t-Bu | t-Bu | 1,3- | CN | CN |
| | | | di-t- | | |
| | | | BuPh | | |
| 115087 | t-Bu | t-Bu | 9-Cbz | CN | CN |
| 115088 | t-Bu | t-Bu | Ph | 9-Cbz | 9-Cbz |
| 115089 | t-Bu | t-Bu | CN | 9-Cbz | 9-Cbz |
| 115090 | t-Bu | t-Bu | DPA | 9-Cbz | 9-Cbz |
| 115091 | t-Bu | t-Bu | 2- | 9-Cbz | 9-Cbz |
| | | | DBF | | |
| 115092 | t-Bu | t-Bu | 2- | 9-Cbz | 9-Cbz |
| | | | DBT | | |
| 115093 | t-Bu | t-Bu | 4-t- | 9-Cbz | 9-Cbz |
| | | | BuPh | | |
| 115094 | t-Bu | t-Bu | 1,3- | 9-Cbz | 9-Cbz |
| | | | di-t- | | |
| | | | BuPh | | |
| 115095 | t-Bu | t-Bu | 9-Cbz | 9-Cbz | 9-Cbz |
| 115096 | t-Bu | t-Bu | Ph | 4-t- | 4-t- |
| | | | | BuPh | BuPh |
| 115097 | t-Bu | t-Bu | CN | 4-t- | 4-t- |
| | | | | BuPh | BuPh |
| 115098 | t-Bu | t-Bu | DPA | 4-t- | 4-t- |
| | | | | BuPh | BuPh |
| 115099 | t-Bu | t-Bu | 2- | 4-t- | 4-t- |
| | | | DBF | BuPh | BuPh |
| 115100 | t-Bu | t-Bu | 2- | 4-t- | 4-t- |
| | | | DBT | BuPh | BuPh |
| 115101 | t-Bu | t-Bu | 4-t- | 4-t- | 4-t- |
| | | | BuPh | BuPh | BuPh |
| 115102 | t-Bu | t-Bu | 1,3- | 4-t- | 4-t- |
| | | | di-t- | BuPh | BuPh |
| | | | BuPh | | |
| 115103 | t-Bu | t-Bu | 9-Cbz | 4-t- | 4-t- |
| | | | | BuPh | BuPh |
| 115104 | t-Bu | t-Bu | t-Bu | 1,3- | 1,3- |
| | | | | di-t- | di-t- |
| | | | | BuPh | BuPh |
| 115105 | t-Bu | t-Bu | Ph | 1,3- | 1,3- |
| | | | | di-t- | di-t- |
| | | | | BuPh | BuPh |
| 115106 | t-Bu | t-Bu | CN | 1,3- | 1,3- |
| | | | | di-t- | di-t- |
| | | | | BuPh | BuPh |
| 115107 | t-Bu | t-Bu | DPA | 1,3- | 1,3- |
| | | | | di-t- | di-t- |
| | | | | BuPh | BuPh |
| 115108 | t-Bu | t-Bu | 2- | 1,3- | 1,3- |
| | | | DBF | di-t- | di-t- |
| | | | | BuPh | BuPh |
| 115109 | t-Bu | t-Bu | 2- | 1,3- | 1,3- |
| | | | DBT | di-t- | di-t- |
| | | | | BuPh | BuPh |
| 115110 | t-Bu | t-Bu | 4-t- | 1,3- | 1,3- |
| | | | BuPh | di-t- | di-t- |
| | | | | BuPh | BuPh |
| 115111 | t-Bu | t-Bu | 1,3- | 1,3- | 1,3- |
| | | | di-t- | di-t- | di-t- |
| | | | BuPh | BuPh | BuPh |
| 115112 | t-Bu | t-Bu | 9-Cbz | 1,3- | 1,3- |
| | | | | di-t- | di-t- |
| | | | | BuPh | BuPh |
| 115113 | Ph | Ph | t-Bu | H | H |
| 115114 | Ph | Ph | CN | H | H |
| 115115 | Ph | Ph | DPA | H | H |
| 115116 | Ph | Ph | 2- | H | H |
| | | | DBF | | |
| 115117 | Ph | Ph | 2- | H | H |
| | | | DBT | | |
| 115118 | Ph | Ph | 4-t- | H | H |
| | | | BuPh | | |
| 115119 | Ph | Ph | 1,3- | H | H |
| | | | di-t- | | |
| | | | BuPh | | |
| 115120 | Ph | Ph | 9-Cbz | H | H |
| 115121 | Ph | Ph | Ph | t-Bu | t-Bu |
| 115122 | Ph | Ph | CN | t-Bu | t-Bu |
| 115123 | Ph | Ph | DPA | t-Bu | t-Bu |
| 115124 | Ph | Ph | 2- | t-Bu | t-Bu |
| | | | DBF | | |
| 115125 | Ph | Ph | 2- | t-Bu | t-Bu |
| | | | DBT | | |
| 115126 | Ph | Ph | 4-t- | t-Bu | t-Bu |
| | | | BuPh | | |
| 115127 | Ph | Ph | 1,3- | t-Bu | t-Bu |
| | | | di-t- | | |
| | | | BuPh | | |
| 115128 | Ph | Ph | 9-Cbz | t-Bu | t-Bu |
| 115129 | Ph | Ph | Ph | Ph | Ph |
| 115130 | Ph | Ph | CN | Ph | Ph |
| 115131 | Ph | Ph | DPA | Ph | Ph |
| 115132 | Ph | Ph | 2- | Ph | Ph |
| | | | DBF | | |
| 115133 | Ph | Ph | 2- | Ph | Ph |
| | | | DBT | | |
| 115134 | Ph | Ph | 4-t- | Ph | Ph |
| | | | BuPh | | |
| 115135 | Ph | Ph | 1,3- | Ph | Ph |
| | | | di-t- | | |
| | | | BuPh | | |
| 115136 | Ph | Ph | 9-Cbz | Ph | Ph |
| 115137 | Ph | Ph | t-Bu | CN | CN |
| 115138 | Ph | Ph | Ph | CN | CN |
| 115139 | Ph | Ph | CN | CN | CN |
| 115140 | Ph | Ph | DPA | CN | CN |
| 115141 | Ph | Ph | 2- | CN | CN |
| | | | DBF | | |
| 115142 | Ph | Ph | 2- | CN | CN |
| | | | DBT | | |
| 115143 | Ph | Ph | 4-t- | CN | CN |
| | | | BuPh | | |
| 115144 | Ph | Ph | 1,3- | CN | CN |
| | | | di-t- | | |
| | | | BuPh | | |
| 115145 | Ph | Ph | 9-Cbz | CN | CN |
| 115146 | Ph | Ph | t-Bu | 9-Cbz | 9-Cbz |
| 115147 | Ph | Ph | Ph | 9-Cbz | 9-Cbz |
| 115148 | Ph | Ph | CN | 9-Cbz | 9-Cbz |
| 115149 | Ph | Ph | DPA | 9-Cbz | 9-Cbz |
| 115150 | Ph | Ph | 2- | 9-Cbz | 9-Cbz |
| | | | DBF | | |
| 115151 | Ph | Ph | 2- | 9-Cbz | 9-Cbz |
| | | | DBT | | |
| 115152 | Ph | Ph | 4-t- | 9-Cbz | 9-Cbz |
| | | | BuPh | | |
| 115153 | Ph | Ph | 1,3- | 9-Cbz | 9-Cbz |
| | | | di-t- | | |
| | | | BuPh | | |
| 115154 | Ph | Ph | 9-Cbz | 9-Cbz | 9-Cbz |
| 115155 | Ph | Ph | t-Bu | 4-t- | 4-t- |
| | | | | BuPh | BuPh |
| 115156 | Ph | Ph | Ph | 4-t- | 4-t- |
| | | | | BuPh | BuPh |
| 115157 | Ph | Ph | CN | 4-t- | 4-t- |
| | | | | BuPh | BuPh |
| 115158 | Ph | Ph | DPA | 4-t- | 4-t- |
| | | | | BuPh | BuPh |
| 115159 | Ph | Ph | 2- | 4-t- | 4-t- |
| | | | DBF | BuPh | BuPh |
| 115160 | Ph | Ph | 2- | 4-t- | 4-t- |
| | | | DBT | BuPh | BuPh |
| 115161 | Ph | Ph | 4-t- | 4-t- | 4-t- |
| | | | BuPh | BuPh | BuPh |
| 115162 | Ph | Ph | 1,3- | 4-t- | 4-t- |
| | | | di-t- | BuPh | BuPh |
| | | | BuPh | | |
| 115163 | Ph | Ph | 9-Cbz | 4-t- | 4-t- |
| | | | | BuPh | BuPh |
| 115164 | Ph | Ph | t-Bu | 1,3- | 1,3- |
| | | | | di-t- | di-t- |
| | | | | BuPh | BuPh |
| 115165 | Ph | Ph | Ph | 1,3- | 1,3- |
| | | | | di-t- | di-t- |
| | | | | BuPh | BuPh |
| 115166 | Ph | Ph | CN | 1,3- | 1,3- |
| | | | | di-t- | di-t- |
| | | | | BuPh | BuPh |
| 115167 | Ph | Ph | DPA | 1,3- | 1,3- |
| | | | | di-t- | di-t- |
| | | | | BuPh | BuPh |
| 115168 | Ph | Ph | 2- | 1,3- | 1,3- |
| | | | DBF | di-t- | di-t- |
| | | | | BuPh | BuPh |
| 115169 | Ph | Ph | 2- | 1,3- | 1,3- |
| | | | DBT | di-t- | di-t- |
| | | | | BuPh | BuPh |
| 115170 | Ph | Ph | 4-t- | 1,3- | 1,3- |
| | | | BuPh | di-t- | di-t- |
| | | | | BuPh | BuPh |
| 115171 | Ph | Ph | 1,3- | 1,3- | 1,3- |
| | | | di-t- | di-t- | di-t- |
| | | | BuPh | BuPh | BuPh |
| 115172 | Ph | Ph | 9-Cbz | 1,3- | 1,3- |
| | | | | di-t- | di-t- |
| | | | | BuPh | BuPh |
| 115173 | 9-Cbz | 9-Cbz | t-Bu | H | H |
| 115174 | 9-Cbz | 9-Cbz | Ph | H | H |
| 115175 | 9-Cbz | 9-Cbz | CN | H | H |
| 115176 | 9-Cbz | 9-Cbz | DPA | H | H |
| 115177 | 9-Cbz | 9-Cbz | 2- | H | H |
| | | | DBF | | |
| 115178 | 9-Cbz | 9-Cbz | 2- | H | H |
| | | | DBT | | |
| 115179 | 9-Cbz | 9-Cbz | 4-t- | H | H |
| | | | BuPh | | |
| 115180 | 9-Cbz | 9-Cbz | 1,3- | H | H |
| | | | di-t- | | |
| | | | BuPh | | |
| 115181 | 9-Cbz | 9-Cbz | t-Bu | t-Bu | t-Bu |
| 115182 | 9-Cbz | 9-Cbz | Ph | t-Bu | t-Bu |
| 115183 | 9-Cbz | 9-Cbz | CN | t-Bu | t-Bu |
| 115184 | 9-Cbz | 9-Cbz | DPA | t-Bu | t-Bu |
| 115185 | 9-Cbz | 9-Cbz | 2- | t-Bu | t-Bu |
| | | | DBF | | |
| 115186 | 9-Cbz | 9-Cbz | 2- | t-Bu | t-Bu |
| | | | DBT | | |
| 115187 | 9-Cbz | 9-Cbz | 4-t- | t-Bu | t-Bu |
| | | | BuPh | | |
| 115188 | 9-Cbz | 9-Cbz | 1,3- | t-Bu | t-Bu |
| | | | di-t- | | |
| | | | BuPh | | |
| 115189 | 9-Cbz | 9-Cbz | 9-Cbz | t-Bu | t-Bu |
| 115190 | 9-Cbz | 9-Cbz | t-Bu | Ph | Ph |
| 115191 | 9-Cbz | 9-Cbz | Ph | Ph | Ph |
| 115192 | 9-Cbz | 9-Cbz | CN | Ph | Ph |
| 115193 | 9-Cbz | 9-Cbz | DPA | Ph | Ph |
| 115194 | 9-Cbz | 9-Cbz | 2- | Ph | Ph |
| | | | DBF | | |
| 115195 | 9-Cbz | 9-Cbz | 2- | Ph | Ph |
| | | | DBT | | |
| 115196 | 9-Cbz | 9-Cbz | 4-t- | Ph | Ph |
| | | | BuPh | | |
| 115197 | 9-Cbz | 9-Cbz | 1,3- | Ph | Ph |
| | | | di-t- | | |
| | | | BuPh | | |
| 115198 | 9-Cbz | 9-Cbz | 9-Cbz | Ph | Ph |
| 115199 | 9-Cbz | 9-Cbz | t-Bu | CN | CN |
| 115200 | 9-Cbz | 9-Cbz | Ph | CN | CN |
| 115201 | 9-Cbz | 9-Cbz | CN | CN | CN |
| 115202 | 9-Cbz | 9-Cbz | DPA | CN | CN |
| 115203 | 9-Cbz | 9-Cbz | 2- | CN | CN |
| | | | DBF | | |
| 115204 | 9-Cbz | 9-Cbz | 2- | CN | CN |
| | | | DBT | | |
| 115205 | 9-Cbz | 9-Cbz | 4-t- | CN | CN |
| | | | BuPh | | |
| 115206 | 9-Cbz | 9-Cbz | 1,3- | CN | CN |
| | | | di-t- | | |
| | | | BuPh | | |
| 115207 | 9-Cbz | 9-Cbz | 9-Cbz | CN | CN |
| 115208 | 9-Cbz | 9-Cbz | t-Bu | 9-Cbz | 9-Cbz |
| 115209 | 9-Cbz | 9-Cbz | Ph | 9-Cbz | 9-Cbz |
| 115210 | 9-Cbz | 9-Cbz | CN | 9-Cbz | 9-Cbz |
| 115211 | 9-Cbz | 9-Cbz | DPA | 9-Cbz | 9-Cbz |
| 115212 | 9-Cbz | 9-Cbz | 2- | 9-Cbz | 9-Cbz |
| | | | dbf | | |
| 115213 | 9-Cbz | 9-Cbz | 2- | 9-Cbz | 9-Cbz |
| | | | dbt | | |
| 115214 | 9-Cbz | 9-Cbz | 4-t- | 9-Cbz | 9-Cbz |
| | | | BuPh | | |
| 115215 | 9-Cbz | 9-Cbz | 1,3- | 9-Cbz | 9-Cbz |
| | | | di-t- | | |
| | | | BuPh | | |
| 115216 | 9-Cbz | 9-Cbz | 9-Cbz | 9-Cbz | 9-Cbz |
| 115217 | 9-Cbz | 9-Cbz | t-Bu | 4-t- | 4-t- |
| | | | | BuPh | BuPh |
| 115218 | 9-Cbz | 9-Cbz | Ph | 4-t- | 4-t- |
| | | | | BuPh | BuPh |
| 115219 | 9-Cbz | 9-Cbz | CN | 4-t- | 4-t- |
| | | | | BuPh | BuPh |
| 115220 | 9-Cbz | 9-Cbz | DPA | 4-t- | 4-t- |
| | | | | BuPh | BuPh |
| 115221 | 9-Cbz | 9-Cbz | 2- | 4-t- | 4-t- |
| | | | DBF | BuPh | BuPh |
| 115222 | 9-Cbz | 9-Cbz | 2- | 4-t- | 4-t- |
| | | | DBT | BuPh | BuPh |
| 115223 | 9-Cbz | 9-Cbz | 4-t- | 4-t- | 4-t- |
| | | | BuPh | BuPh | BuPh |
| 115224 | 9-Cbz | 9-Cbz | 1,3- | 4-t- | 4-t- |
| | | | di-t- | BuPh | BuPh |
| | | | BuPh | | |
| 115225 | 9-Cbz | 9-Cbz | 9-Cbz | 4-t- | 4-t- |
| | | | | BuPh | BuPh |
| 115226 | 9-Cbz | 9-Cbz | t-Bu | 1,3- | 1,3- |
| | | | | di-t- | di-t- |
| | | | | BuPh | BuPh |
| 115227 | 9-Cbz | 9-Cbz | Ph | 1,3- | 1,3- |
| | | | | di-t- | di-t- |
| | | | | BuPh | BuPh |
| 115228 | 9-Cbz | 9-Cbz | CN | 1,3- | 1,3- |
| | | | | di-t- | di-t- |
| | | | | BuPh | BuPh |
| 115229 | 9-Cbz | 9-Cbz | DPA | 1,3- | 1,3- |
| | | | | di-t- | di-t- |
| | | | | BuPh | BuPh |
| 115230 | 9-Cbz | 9-Cbz | 2- | 1,3- | 1,3- |
| | | | DBF | di-t- | di-t- |
| | | | | BuPh | BuPh |
| 115231 | 9-Cbz | 9-Cbz | 2- | 1,3- | 1,3- |
| | | | DBT | di-t- | di-t- |
| | | | | BuPh | BuPh |
| 115232 | 9-Cbz | 9-Cbz | 4-t- | 1,3- | 1,3- |
| | | | BuPh | di-t- | di-t- |
| | | | | BuPh | BuPh |
| 115233 | 9-Cbz | 9-Cbz | 1,3- | 1,3- | 1,3- |
| | | | di-t- | di-t- | di-t- |
| | | | BuPh | BuPh | BuPh |
| 115234 | 9-Cbz | 9-Cbz | 9-Cbz | 1,3- | 1,3- |
| | | | | di-t- | di-t- |
| | | | | BuPh | BuPh |
| 115235 | CN | CN | t-Bu | H | H |
| 115236 | CN | CN | Ph | H | H |
| 115237 | CN | CN | DPA | H | H |
| 115238 | CN | CN | 2- | H | H |
| | | | DBF | | |
| 115239 | CN | CN | 2- | H | H |
| | | | DBT | | |
| 115240 | CN | CN | 4-t- | H | H |
| | | | BuPh | | |
| 115241 | CN | CN | 1,3- | H | H |
| | | | di-t- | | |
| | | | BuPh | | |
| 115242 | CN | CN | 9-Cbz | H | H |
| 115243 | CN | CN | t-Bu | t-Bu | t-Bu |
| 115244 | CN | CN | Ph | t-Bu | t-Bu |
| 115245 | CN | CN | CN | t-Bu | t-Bu |
| 115246 | CN | CN | DPA | t-Bu | t-Bu |
| 115247 | CN | CN | 2- | t-Bu | t-Bu |
| | | | DBF | | |
| 115248 | CN | CN | 2- | t-Bu | t-Bu |
| | | | DBT | | |
| 115249 | CN | CN | 4-t- | t-Bu | t-Bu |
| | | | BuPh | | |
| 115250 | CN | CN | 1,3- | t-Bu | t-Bu |
| | | | di-t- | | |
| | | | BuPh | | |
| 115251 | CN | CN | 9-Cbz | t-Bu | t-Bu |
| 115252 | CN | CN | t-Bu | Ph | Ph |
| 115253 | CN | CN | Ph | Ph | Ph |
| 115254 | CN | CN | CN | Ph | Ph |
| 115255 | CN | CN | DPA | Ph | Ph |
| 115256 | CN | CN | 2- | Ph | Ph |
| | | | DBF | | |
| 115257 | CN | CN | 2- | Ph | Ph |
| | | | DBT | | |
| 115258 | CN | CN | 4-t- | Ph | Ph |
| | | | BuPh | | |
| 115259 | CN | CN | 1,3- | Ph | Ph |
| | | | di-t- | | |
| | | | BuPh | | |
| 115260 | CN | CN | 9-Cbz | Ph | Ph |
| 115261 | CN | CN | t-Bu | CN | CN |
| 115262 | CN | CN | Ph | CN | CN |
| 115263 | CN | CN | CN | CN | CN |
| 115264 | CN | CN | DPA | CN | CN |
| 115265 | CN | CN | 2- | CN | CN |
| | | | DBF | | |
| 115266 | CN | CN | 2- | CN | CN |
| | | | DBT | | |
| 115267 | CN | CN | 4-t- | CN | CN |
| | | | BuPh | | |
| 115268 | CN | CN | 1,3- | CN | CN |
| | | | di-t- | | |
| | | | BuPh | | |
| 115269 | CN | CN | 9-Cbz | CN | CN |
| 115270 | CN | CN | t-Bu | 9-Cbz | 9-Cbz |
| 115271 | CN | CN | Ph | 9-Cbz | 9-Cbz |
| 115272 | CN | CN | CN | 9-Cbz | 9-Cbz |
| 115273 | CN | CN | DPA | 9-Cbz | 9-Cbz |
| 115274 | CN | CN | 2- | 9-Cbz | 9-Cbz |
| | | | DBF | | |
| 115275 | CN | CN | 2- | 9-Cbz | 9-Cbz |
| | | | DBT | | |
| 115276 | CN | CN | 4-t- | 9-Cbz | 9-Cbz |
| | | | BuPh | | |
| 115277 | CN | CN | 1,3- | 9-Cbz | 9-Cbz |
| | | | di-t- | | |
| | | | BuPh | | |
| 115278 | CN | CN | 9-Cbz | 9-Cbz | 9-Cbz |
| 115279 | CN | CN | t-Bu | 4-t- | 4-t- |
| | | | | BuPh | BuPh |
| 115280 | CN | CN | Ph | 4-t- | 4-t- |
| | | | | BuPh | BuPh |
| 115281 | CN | CN | CN | 4-t- | 4-t- |
| | | | | BuPh | BuPh |
| 115282 | CN | CN | DPA | 4-t- | 4-t- |
| | | | | BuPh | BuPh |
| 115283 | CN | CN | 2- | 4-t- | 4-t- |
| | | | DBF | BuPh | BuPh |
| 115284 | CN | CN | 2- | 4-t- | 4-t- |
| | | | DBT | BuPh | BuPh |
| 115285 | CN | CN | 4-t- | 4-t- | 4-t- |
| | | | BuPh | BuPh | BuPh |
| 115286 | CN | CN | 1,3- | 4-t- | 4-t- |
| | | | di-t- | BuPh | BuPh |
| | | | BuPh | | |
| 115287 | CN | CN | 9-Cbz | 4-t- | 4-t- |
| | | | | BuPh | BuPh |
| 115288 | CN | CN | t-Bu | 1,3- | 1,3- |
| | | | | di-t- | di-t- |
| | | | | BuPh | BuPh |
| 115289 | CN | CN | Ph | 1,3- | 1,3- |
| | | | | di-t- | di-t- |
| | | | | BuPh | BuPh |
| 115290 | CN | CN | CN | 1,3- | 1,3- |
| | | | | di-t- | di-t- |
| | | | | BuPh | BuPh |
| 115291 | CN | CN | DPA | 1,3- | 1,3- |
| | | | | di-t- | di-t- |
| | | | | BuPh | BuPh |
| 115292 | CN | CN | 2- | 1,3- | 1,3- |
| | | | DBF | di-t- | di-t- |
| | | | | BuPh | BuPh |
| 115293 | CN | CN | 2- | 1,3- | 1,3- |
| | | | DBT | di-t- | di-t- |
| | | | | BuPh | BuPh |
| 115294 | CN | CN | 4-t- | 1,3- | 1,3- |
| | | | BuPh | di-t- | di-t- |
| | | | | BuPh | BuPh |
| 115295 | CN | CN | 1,3- | 1,3- | 1,3- |
| | | | di-t- | di-t- | di-t- |
| | | | BuPh | BuPh | BuPh |
| 115296 | CN | CN | 9-Cbz | 1,3- | 1,3- |
| | | | | di-t- | di-t- |
| | | | | BuPh | BuPh |
| 115297 | 4-t- | 4-t- | t-Bu | H | H |
| | BuPh | BuPh | | | |
| 115298 | 4-t- | 4-t- | Ph | H | H |
| | BuPh | BuPh | | | |
| 115299 | 4-t- | 4-t- | CN | H | H |
| | BuPh | BuPh | | | |
| 115300 | 4-t- | 4-t- | DPA | H | H |
| | BuPh | BuPh | | | |
| 115301 | 4-t- | 4-t- | 2- | H | H |
| | BuPh | BuPh | DBF | | |
| 115302 | 4-t- | 4-t- | 2- | H | H |
| | BuPh | BuPh | DBT | | |
| 115303 | 4-t- | 4-t- | 1,3- | H | H |
| | BuPh | BuPh | di-t- | | |
| | | | BuPh | | |
| 115304 | 4-t- | 4-t- | 9-Cbz | H | H |
| | BuPh | BuPh | | | |
| 115305 | 4-t- | 4-t- | t-Bu | t-Bu | t-Bu |
| | BuPh | BuPh | | | |
| 115306 | 4-t- | 4-t- | Ph | t-Bu | t-Bu |
| | BuPh | BuPh | | | |
| 115307 | 4-t- | 4-t- | CN | t-Bu | t-Bu |
| | BuPh | BuPh | | | |
| 115308 | 4-t- | 4-t- | DPA | t-Bu | t-Bu |
| | BuPh | BuPh | | | |
| 115309 | 4-t- | 4-t- | 2- | t-Bu | t-Bu |
| | BuPh | BuPh | DBF | | |
| 115310 | 4-t- | 4-t- | 2- | t-Bu | t-Bu |
| | BuPh | BuPh | DBT | | |
| 115311 | 4-t- | 4-t- | 4-t- | t-Bu | t-Bu |
| | BuPh | BuPh | BuPh | | |
| 115312 | 4-t- | 4-t- | 1,3- | t-Bu | t-Bu |
| | BuPh | BuPh | di-t- | | |
| | | | BuPh | | |
| 115313 | 4-t- | 4-t- | 9-Cbz | t-Bu | t-Bu |
| | BuPh | BuPh | | | |
| 115314 | 4-t- | 4-t- | t-Bu | Ph | Ph |
| | BuPh | BuPh | | | |
| 115315 | 4-t- | 4-t- | Ph | Ph | Ph |
| | BuPh | BuPh | | | |
| 115316 | 4-t- | 4-t- | CN | Ph | Ph |
| | BuPh | BuPh | | | |
| 115317 | 4-t- | 4-t- | DPA | Ph | Ph |
| | BuPh | BuPh | | | |
| 115318 | 4-t- | 4-t- | 2- | Ph | Ph |
| | BuPh | BuPh | DBF | | |
| 115319 | 4-t- | 4-t- | 2- | Ph | Ph |
| | BuPh | BuPh | DBT | | |
| 115320 | 4-t- | 4-t- | 4-t- | Ph | Ph |
| | BuPh | BuPh | BuPh | | |
| 115321 | 4-t- | 4-t- | 1,3- | Ph | Ph |
| | BuPh | BuPh | di-t- | | |
| | | | BuPh | | |
| 115322 | 4-t- | 4-t- | 9-Cbz | Ph | Ph |
| | BuPh | BuPh | | | |
| 115323 | 4-t- | 4-t- | t-Bu | CN | CN |
| | BuPh | BuPh | | | |
| 115324 | 4-t- | 4-t- | Ph | CN | CN |
| | BuPh | BuPh | | | |
| 115325 | 4-t- | 4-t- | CN | CN | CN |
| | BuPh | BuPh | | | |
| 115326 | 4-t- | 4-t- | DPA | CN | CN |
| | BuPh | BuPh | | | |
| 115327 | 4-t- | 4-t- | 2- | CN | CN |
| | BuPh | BuPh | DBF | | |
| 115328 | 4-t- | 4-t- | 2- | CN | CN |
| | BuPh | BuPh | DBT | | |
| 115329 | 4-t- | 4-t- | 4-t- | CN | CN |
| | BuPh | BuPh | BuPh | | |
| 115330 | 4-t- | 4-t- | 1,3- | CN | CN |
| | BuPh | BuPh | di-t- | | |
| | | | BuPh | | |
| 115331 | 4-t- | 4-t- | 9-Cbz | CN | CN |
| | BuPh | BuPh | | | |
| 115332 | 4-t- | 4-t- | t-Bu | 9-Cbz | 9-Cbz |
| | BuPh | BuPh | | | |
| 115333 | 4-t- | 4-t- | Ph | 9-Cbz | 9-Cbz |
| | BuPh | BuPh | | | |
| 115334 | 4-t- | 4-t- | CN | 9-Cbz | 9-Cbz |
| | BuPh | BuPh | | | |
| 115335 | 4-t- | 4-t- | DPA | 9-Cbz | 9-Cbz |
| | BuPh | BuPh | | | |
| 115336 | 4-t- | 4-t- | 2- | 9-Cbz | 9-Cbz |
| | BuPh | BuPh | DBF | | |
| 115337 | 4-t- | 4-t- | 2- | 9-Cbz | 9-Cbz |
| | BuPh | BuPh | DBT | | |
| 115338 | 4-t- | 4-t- | 4-t- | 9-Cbz | 9-Cbz |
| | BuPh | BuPh | BuPh | | |
| 115339 | 4-t- | 4-t- | 1,3- | 9-Cbz | 9-Cbz |
| | BuPh | BuPh | di-t- | | |
| | | | BuPh | | |
| 115340 | 4-t- | 4-t- | 9-Cbz | 9-Cbz | 9-Cbz |
| | BuPh | BuPh | | | |
| 115341 | 4-t- | 4-t- | t-Bu | 4-t- | 4-t- |
| | BuPh | BuPh | | BuPh | BuPh |
| 115342 | 4-t- | 4-t- | Ph | 4-t- | 4-t- |
| | BuPh | BuPh | | BuPh | BuPh |
| 115343 | 4-t- | 4-t- | CN | 4-t- | 4-t- |
| | BuPh | BuPh | | BuPh | BuPh |
| 115344 | 4-t- | 4-t- | DPA | 4-t- | 4-t- |
| | BuPh | BuPh | | BuPh | BuPh |
| 115345 | 4-t- | 4-t- | 2- | 4-t- | 4-t- |
| | BuPh | BuPh | DBF | BuPh | BuPh |
| 115346 | 4-t- | 4-t- | 2- | 4-t- | 4-t- |
| | BuPh | BuPh | DBT | BuPh | BuPh |
| 115347 | 4-t- | 4-t- | 4-t- | 4-t- | 4-t- |
| | BuPh | BuPh | BuPh | BuPh | BuPh |
| 115348 | 4-t- | 4-t- | 1,3- | 4-t- | 4-t- |
| | BuPh | BuPh | di-t- | BuPh | BuPh |
| | | | BuPh | | |
| 115349 | 4-t- | 4-t- | 9-Cbz | 4-t- | 4-t- |
| | BuPh | BuPh | | BuPh | BuPh |
| 115350 | 4-t- | 4-t- | t-Bu | 1,3- | 1,3- |
| | BuPh | BuPh | | di-t- | di-t- |
| | | | | BuPh | BuPh |
| 115351 | 4-t- | 4-t- | Ph | 1,3- | 1,3- |
| | BuPh | BuPh | | di-t- | di-t- |
| | | | | BuPh | BuPh |
| 115352 | 4-t- | 4-t- | CN | 1,3- | 1,3- |
| | BuPh | BuPh | | di-t- | di-t- |
| | | | | BuPh | BuPh |
| 115353 | 4-t- | 4-t- | DPA | 1,3- | 1,3- |
| | BuPh | BuPh | | di-t- | di-t- |
| | | | | BuPh | BuPh |
| 115354 | 4-t- | 4-t- | 2- | 1,3- | 1,3- |
| | BuPh | BuPh | DBF | di-t- | di-t- |
| | | | | BuPh | BuPh |
| 115355 | 4-t- | 4-t- | 2- | 1,3- | 1,3- |
| | BuPh | BuPh | DBT | di-t- | di-t- |
| | | | | BuPh | BuPh |
| 115356 | 4-t- | 4-t- | 4-t- | 1,3- | 1,3- |
| | BuPh | BuPh | BuPh | di-t- | di-t- |
| | | | | BuPh | BuPh |
| 115357 | 4-t- | 4-t- | 1,3- | 1,3- | 1,3- |
| | BuPh | BuPh | di-t- | di-t- | di-t- |
| | | | BuPh | BuPh | BuPh |
| 115358 | 4-t- | 4-t- | 9-Cbz | 1,3- | 1,3- |
| | BuPh | BuPh | | di-t- | di-t- |
| | | | | BuPh | BuPh |
| 115359 | 1,3- | 1,3- | t-Bu | H | H |
| | di-t- | di-t- | | | |
| | BuPh | BuPh | | | |
| 115360 | 1,3- | 1,3- | Ph | H | H |
| | di-t- | di-t- | | | |
| | BuPh | BuPh | | | |
| 115361 | 1,3- | 1,3- | CN | H | H |
| | di-t- | di-t- | | | |
| | BuPh | BuPh | | | |
| 115362 | 1,3- | 1,3- | DPA | H | H |
| | di-t- | di-t- | | | |
| | BuPh | BuPh | | | |
| 115363 | 1,3- | 1,3- | 2- | H | H |
| | di-t- | di-t- | DBF | | |
| | BuPh | BuPh | | | |
| 115364 | 1,3- | 1,3- | 2- | H | H |
| | di-t- | di-t- | DBT | | |
| | BuPh | BuPh | | | |
| 115365 | 1,3- | 1,3- | 4-t- | H | H |
| | di-t- | di-t- | BuPh | | |
| | BuPh | BuPh | | | |
| 115366 | 1,3- | 1,3- | 9-Cbz | H | H |
| | di-t- | di-t- | | | |
| | BuPh | BuPh | | | |
| 115367 | 1,3- | 1,3- | t-Bu | t-Bu | t-Bu |
| | di-t- | di-t- | | | |
| | BuPh | BuPh | | | |
| 115368 | 1,3- | 1,3- | Ph | t-Bu | t-Bu |
| | di-t- | di-t- | | | |
| | BuPh | BuPh | | | |
| 115369 | 1,3- | 1,3- | CN | t-Bu | t-Bu |
| | di-t- | di-t- | | | |
| | BuPh | BuPh | | | |
| 115370 | 1,3- | 1,3- | DPA | t-Bu | t-Bu |
| | di-t- | di-t- | | | |
| | BuPh | BuPh | | | |
| 115371 | 1,3- | 1,3- | 2- | t-Bu | t-Bu |
| | di-t- | di-t- | DBF | | |
| | BuPh | BuPh | | | |
| 115372 | 1,3- | 1,3- | 2- | t-Bu | t-Bu |
| | di-t- | di-t- | DBT | | |
| | BuPh | BuPh | | | |
| 115373 | 1,3- | 1,3- | 4-t- | t-Bu | t-Bu |
| | di-t- | di-t- | BuPh | | |
| | BuPh | BuPh | | | |
| 115374 | 1,3- | 1,3- | 1,3- | t-Bu | t-Bu |
| | di-t- | di-t- | di-t- | | |
| | BuPh | BuPh | BuPh | | |
| 115375 | 1,3- | 1,3- | 9-Cbz | t-Bu | t-Bu |
| | di-t- | di-t- | | | |
| | BuPh | BuPh | | | |
| 115376 | 1,3- | 1,3- | t-Bu | Ph | Ph |
| | di-t- | di-t- | | | |
| | BuPh | BuPh | | | |
| 115377 | 1,3- | 1,3- | Ph | Ph | Ph |
| | di-t- | di-t- | | | |
| | BuPh | BuPh | | | |
| 115378 | 1,3- | 1,3- | CN | Ph | Ph |
| | di-t- | di-t- | | | |
| | BuPh | BuPh | | | |
| 115379 | 1,3- | 1,3- | DPA | Ph | Ph |
| | di-t- | di-t- | | | |
| | BuPh | BuPh | | | |
| 115380 | 1,3- | 1,3- | 2- | Ph | Ph |
| | di-t- | di-t- | DBF | | |
| | BuPh | BuPh | | | |
| 115381 | 1,3- | 1,3- | 2- | Ph | Ph |
| | di-t- | di-t- | DBT | | |
| | BuPh | BuPh | | | |
| 115382 | 1,3- | 1,3- | 4-t- | Ph | Ph |
| | di-t- | di-t- | BuPh | | |
| | BuPh | BuPh | | | |
| 115383 | 1,3- | 1,3- | 1,3- | Ph | Ph |
| | di-t- | di-t- | di-t- | | |
| | BuPh | BuPh | BuPh | | |
| 115384 | 1,3- | 1,3- | 9-Cbz | Ph | Ph |
| | di-t- | di-t- | | | |
| | BuPh | BuPh | | | |
| 115385 | 1,3- | 1,3- | t-Bu | CN | CN |
| | di-t- | di-t- | | | |
| | BuPh | BuPh | | | |
| 115386 | 1,3- | 1,3- | Ph | CN | CN |
| | di-t- | di-t- | | | |
| | BuPh | BuPh | | | |
| 115387 | 1,3- | 1,3- | CN | CN | CN |
| | di-t- | di-t- | | | |
| | BuPh | BuPh | | | |
| 115388 | 1,3- | 1,3- | DPA | CN | CN |
| | di-t- | di-t- | | | |
| | BuPh | BuPh | | | |
| 115389 | 1,3- | 1,3- | 2- | CN | CN |
| | di-t- | di-t- | DBF | | |
| | BuPh | BuPh | | | |
| 115390 | 1,3- | 1,3- | 2- | CN | CN |
| | di-t- | di-t- | DBT | | |
| | BuPh | BuPh | | | |
| 115391 | 1,3- | 1,3- | 4-t- | CN | CN |
| | di-t- | di-t- | BuPh | | |
| | BuPh | BuPh | | | |
| 115392 | 1,3- | 1,3- | 1,3- | CN | CN |
| | di-t- | di-t- | di-t- | | |
| | BuPh | BuPh | BuPh | | |
| 115393 | 1,3- | 1,3- | 9-Cbz | CN | CN |
| | di-t- | di-t- | | | |
| | BuPh | BuPh | | | |
| 115394 | 1,3- | 1,3- | t-Bu | 9-Cbz | 9-Cbz |
| | di-t- | di-t- | | | |
| | BuPh | BuPh | | | |
| 115395 | 1,3- | 1,3- | Ph | 9-Cbz | 9-Cbz |
| | di-t- | di-t- | | | |
| | BuPh | BuPh | | | |
| 115396 | 1,3- | 1,3- | CN | 9-Cbz | 9-Cbz |
| | di-t- | di-t- | | | |
| | BuPh | BuPh | | | |
| 115397 | 1,3- | 1,3- | DPA | 9-Cbz | 9-Cbz |
| | di-t- | di-t- | | | |
| | BuPh | BuPh | | | |
| 115398 | 1,3- | 1,3- | 2- | 9-Cbz | 9-Cbz |
| | di-t- | di-t- | DBF | | |
| | BuPh | BuPh | | | |
| 115399 | 1,3- | 1,3- | 2- | 9-Cbz | 9-Cbz |
| | di-t- | di-t- | DBT | | |
| | BuPh | BuPh | | | |
| 115400 | 1,3- | 1,3- | 4-t- | 9-Cbz | 9-Cbz |
| | di-t- | di-t- | BuPh | | |
| | BuPh | BuPh | | | |
| 115401 | 1,3- | 1,3- | 1,3- | 9-Cbz | 9-Cbz |
| | di-t- | di-t- | di-t- | | |
| | BuPh | BuPh | BuPh | | |
| 115402 | 1,3- | 1,3- | 9-Cbz | 9-Cbz | 9-Cbz |
| | di-t- | di-t- | | | |
| | BuPh | BuPh | | | |
| 115403 | 1,3- | 1,3- | t-Bu | 4-t- | 4-t- |
| | di-t- | di-t- | | BuPh | BuPh |
| | BuPh | BuPh | | | |
| 115404 | 1,3- | 1,3- | Ph | 4-t- | 4-t- |
| | di-t- | di-t- | | BuPh | BuPh |
| | BuPh | BuPh | | | |
| 115405 | 1,3- | 1,3- | CN | 4-t- | 4-t- |
| | di-t- | di-t- | | BuPh | BuPh |
| | BuPh | BuPh | | | |
| 115406 | 1,3- | 1,3- | DPA | 4-t- | 4-t- |
| | di-t- | di-t- | | BuPh | BuPh |
| | BuPh | BuPh | | | |
| 115407 | 1,3- | 1,3- | 2- | 4-t- | 4-t- |
| | di-t- | di-t- | DBF | BuPh | BuPh |
| | BuPh | BuPh | | | |
| 115408 | 1,3- | 1,3- | 2- | 4-t- | 4-t- |
| | di-t- | di-t- | DBT | BuPh | BuPh |
| | BuPh | BuPh | | | |
| 115409 | 1,3- | 1,3- | 4-t- | 4-t- | 4-t- |
| | di-t- | di-t- | BuPh | BuPh | BuPh |
| | BuPh | BuPh | | | |
| 115410 | 1,3- | 1,3- | 1,3- | 4-t- | 4-t- |
| | di-t- | di-t- | di-t- | BuPh | BuPh |
| | BuPh | BuPh | BuPh | | |
| 115411 | 1,3- | 1,3- | 9-Cbz | 4-t- | 4-t- |
| | di-t- | di-t- | | BuPh | BuPh |
| | BuPh | BuPh | | | |
| 115412 | 1,3- | 1,3- | t-Bu | 1,3- | 1,3- |
| | di-t- | di-t- | | di-t- | di-t- |
| | BuPh | BuPh | | BuPh | BuPh |
| 115413 | 1,3- | 1,3- | Ph | 1,3- | 1,3- |
| | di-t- | di-t- | | di-t- | di-t- |
| | BuPh | BuPh | | BuPh | BuPh |
| 115414 | 1,3- | 1,3- | CN | 1,3- | 1,3- |
| | di-t- | di-t- | | di-t- | di-t- |
| | BuPh | BuPh | | BuPh | BuPh |
| 115415 | 1,3- | 1,3- | DPA | 1,3- | 1,3- |
| | di-t- | di-t- | | di-t- | di-t- |
| | BuPh | BuPh | | BuPh | BuPh |
| 115416 | 1,3- | 1,3- | 2- | 1,3- | 1,3- |
| | di-t- | di-t- | DBF | di-t- | di-t- |
| | BuPh | BuPh | | BuPh | BuPh |
| 115417 | 1,3- | 1,3- | 2- | 1,3- | 1,3- |
| | di-t- | di-t- | DBT | di-t- | di-t- |
| | BuPh | BuPh | | BuPh | BuPh |
| 115418 | 1,3- | 1,3- | 4-t- | 1,3- | 1,3- |
| | di-t- | di-t- | BuPh | di-t- | di-t- |
| | BuPh | BuPh | | BuPh | BuPh |
| 115419 | 1,3- | 1,3- | 1,3- | 1,3- | 1,3- |
| | di-t- | di-t- | di-t- | di-t- | di-t- |
| | BuPh | BuPh | BuPh | BuPh | BuPh |
| 115420 | 1,3- | 1,3- | 9-Cbz | 1,3- | 1,3- |
| | di-t- | di-t- | | di-t- | di-t- |
| | BuPh | BuPh | | BuPh | BuPh |

### Group HC3

The heterocyclic compound may be represented by Formula 12-1, and substituents may be defined as shown in Table 3.

TABLE 3

| Compound | | | | | |
|---|---|---|---|---|---|
| No. | R_{11b} | R_{12c} | R_{14b} | R_{18c} | R_{19b} |
| 121001 | t-Bu | t-Bu | t-Bu | t-Bu | t-Bu |
| 121002 | Ph | Ph | t-Bu | Ph | Ph |
| 121003 | t-Bu | t-Bu | H | H | H |
| 121004 | H | H | t-Bu | H | H |
| 121005 | H | H | H | t-Bu | t-Bu |
| 121006 | t-Bu | t-Bu | t-Bu | H | H |
| 121007 | H | H | t-Bu | t-Bu | t-Bu |
| 121008 | t-Bu | t-Bu | H | t-Bu | t-Bu |
| 121009 | Ph | Ph | H | H | H |
| 121010 | H | H | Ph | H | H |
| 121011 | H | H | H | Ph | Ph |
| 121012 | Ph | Ph | Ph | H | H |
| 121013 | H | H | Ph | Ph | Ph |
| 121014 | Ph | Ph | H | Ph | Ph |
| 121015 | CN | CN | H | H | H |
| 121016 | H | H | CN | H | H |
| 121017 | H | H | H | CN | CN |
| 121018 | CN | CN | CN | H | H |
| 121019 | H | H | CN | CN | CN |
| 121020 | CN | CN | H | CN | CN |
| 121021 | 9-Cbz | 9-Cbz | H | H | H |
| 121022 | H | H | 9-Cbz | H | H |
| 121023 | H | H | H | 9-Cbz | 9-Cbz |
| 121024 | 9-Cbz | 9-Cbz | 9-Cbz | H | H |
| 121025 | H | H | 9-Cbz | 9-Cbz | 9-Cbz |
| 121026 | 9-Cbz | 9-Cbz | H | 9-Cbz | 9-Cbz |
| 121027 | Me | Me | H | H | H |
| 121028 | H | H | Me | H | H |
| 121029 | H | H | H | Me | Me |
| 121030 | Me | Me | Me | H | H |
| 121031 | H | H | Me | Me | Me |
| 121032 | Me | Me | H | Me | Me |
| 121033 | CD₃ | CD3 | H | H | H |
| 121034 | H | H | CD3 | H | H |
| 121035 | H | H | H | CD3 | CD3 |
| 121036 | CD3 | CD3 | CD3 | H | H |
| 121037 | H | H | CD3 | CD3 | CD3 |
| 121038 | CD3 | CD3 | H | CD3 | CD3 |
| 121039 | 4-t- | 4-t- | H | H | H |
| | BuPh | BuPh | | | |
| 121040 | H | H | 4-t- | H | H |
| | | | BuPh | | |
| 121041 | H | H | H | 4-t- | 4-t- |
| | | | | BuPh | BuPh |
| 121042 | 4-t- | 4-t- | 4-t- | H | H |
| | BuPh | BuPh | BuPh | | |
| 121043 | H | H | 4-t- | 4-t- | 4-t- |
| | | | BuPh | BuPh | BuPh |
| 121044 | 4-t- | 4-t- | H | 4-t- | 4-t- |
| | BuPh | BuPh | | BuPh | BuPh |
| 121045 | 1,3- | 1,3- | H | H | H |
| | di-t- | di-t- | | | |
| | BuPh | BuPh | | | |
| 121046 | H | H | 1,3- | H | H |
| | | | di-t- | | |
| | | | BuPh | | |
| 121047 | H | H | H | 1,3- | 1,3- |
| | | | | di-t- | di-t- |
| | | | | BuPh | BuPh |
| 121048 | 1,3- | 1,3- | 1,3- | H | H |
| | di-t- | di-t- | di-t- | | |
| | BuPh | BuPh | BuPh | | |
| 121049 | H | H | 1,3- | 1,3- | 1,3- |
| | | | di-t- | di-t- | di-t- |
| | | | BuPh | BuPh | BuPh |
| 121050 | 1,3- | 1,3- | H | 1,3- | 1,3- |
| | di-t- | di-t- | | di-t- | di-t- |
| | BuPh | BuPh | | BuPh | BuPh |
| 121051 | t-Bu | t-Bu | Ph | H | H |
| 121052 | t-Bu | t-Bu | CN | H | H |
| 121053 | t-Bu | t-Bu | DPA | H | H |
| 121054 | t-Bu | t-Bu | 2- | H | H |
| | | | DBF | | |
| 121055 | t-Bu | t-Bu | 2- | H | H |
| | | | DBT | | |
| 121056 | t-Bu | t-Bu | 4-t- | H | H |
| | | | BuPh | | |
| 121057 | t-Bu | t-Bu | 1,3- | H | H |
| | | | di-t- | | |
| | | | BuPh | | |
| 121058 | t-Bu | t-Bu | 9-Cbz | H | H |
| 121059 | t-Bu | t-Bu | Ph | t-Bu | t-Bu |
| 121060 | t-Bu | t-Bu | CN | t-Bu | t-Bu |
| 121061 | t-Bu | t-Bu | DPA | t-Bu | t-Bu |
| 121062 | t-Bu | t-Bu | 2- | t-Bu | t-Bu |
| | | | DBF | | |
| 121063 | t-Bu | t-Bu | 2- | t-Bu | t-Bu |
| | | | DBT | | |
| 121064 | t-Bu | t-Bu | 4-t- | t-Bu | t-Bu |
| | | | BuPh | | |
| 121065 | t-Bu | t-Bu | 1,3- | t-Bu | t-Bu |
| | | | di-t- | | |
| | | | BuPh | | |
| 121066 | t-Bu | t-Bu | 9-Cbz | t-Bu | t-Bu |
| 121067 | t-Bu | t-Bu | t-Bu | Ph | Ph |
| 121068 | t-Bu | t-Bu | Ph | Ph | Ph |
| 121069 | t-Bu | t-Bu | CN | Ph | Ph |
| 121070 | t-Bu | t-Bu | DPA | Ph | Ph |
| 121071 | t-Bu | t-Bu | 2- | Ph | Ph |
| | | | DBF | | |
| 121072 | t-Bu | t-Bu | 2- | Ph | Ph |
| | | | DBT | | |
| 121073 | t-Bu | t-Bu | 4-t- | Ph | Ph |
| | | | BuPh | | |
| 121074 | t-Bu | t-Bu | 1,3- | Ph | Ph |
| | | | di-t- | | |
| | | | BuPh | | |
| 121075 | t-Bu | t-Bu | 9-Cbz | Ph | Ph |
| 121076 | t-Bu | t-Bu | t-Bu | CN | CN |
| 121077 | t-Bu | t-Bu | Ph | CN | CN |
| 121078 | t-Bu | t-Bu | CN | CN | CN |
| 121079 | t-Bu | t-Bu | DPA | CN | CN |
| 121080 | t-Bu | t-Bu | 2- | CN | CN |
| | | | DBF | | |
| 121081 | t-Bu | t-Bu | 2- | CN | CN |
| | | | DBT | | |
| 121082 | t-Bu | t-Bu | 4-t- | CN | CN |
| | | | BuPh | | |
| 121083 | t-Bu | t-Bu | 1,3- | CN | CN |
| | | | di-t- | | |
| | | | BuPh | | |
| 121084 | t-Bu | t-Bu | 9-Cbz | CN | CN |
| 121085 | t-Bu | t-Bu | t-Bu | 9-Cbz | 9-Cbz |
| 121086 | t-Bu | t-Bu | Ph | 9-Cbz | 9-Cbz |
| 121087 | t-Bu | t-Bu | CN | 9-Cbz | 9-Cbz |
| 121088 | t-Bu | t-Bu | DPA | 9-Cbz | 9-Cbz |
| 121089 | t-Bu | t-Bu | 2- | 9-Cbz | 9-Cbz |
| | | | DBF | | |
| 121090 | t-Bu | t-Bu | 2- | 9-Cbz | 9-Cbz |
| | | | DBT | | |
| 121091 | t-Bu | t-Bu | 4-t- | 9-Cbz | 9-Cbz |
| | | | BuPh | | |
| 121092 | t-Bu | t-Bu | 1,3- | 9-Cbz | 9-Cbz |
| | | | di-t- | | |
| | | | BuPh | | |
| 121093 | t-Bu | t-Bu | 9-Cbz | 9-Cbz | 9-Cbz |
| 121094 | t-Bu | t-Bu | t-Bu | 4-t- | 4-t- |
| | | | | BuPh | BuPh |
| 121095 | t-Bu | t-Bu | Ph | 4-t- | 4-t- |
| | | | | BuPh | BuPh |
| 121096 | t-Bu | t-Bu | CN | 4-t- | 4-t- |
| | | | | BuPh | BuPh |
| 121097 | t-Bu | t-Bu | DPA | 4-t- | 4-t- |
| | | | | BuPh | BuPh |
| 121098 | t-Bu | t-Bu | 2- | 4-t- | 4-t- |
| | | | DBF | BuPh | BuPh |
| 121099 | t-Bu | t-Bu | 2- | 4-t- | 4-t- |
| | | | DBT | BuPh | BuPh |
| 121100 | t-Bu | t-Bu | 4-t- | 4-t- | 4-t- |
| | | | BuPh | BuPh | BuPh |
| 121101 | t-Bu | t-Bu | 1,3- | 4-t- | 4-t- |
| | | | di-t- | BuPh | BuPh |
| | | | BuPh | | |
| 121102 | t-Bu | t-Bu | 9-Cbz | 4-t- | 4-t- |
| | | | | BuPh | BuPh |
| 121103 | t-Bu | t-Bu | t-Bu | 1,3- | 1,3- |
| | | | | di-t- | di-t- |
| | | | | BuPh | BuPh |
| 121104 | t-Bu | t-Bu | Ph | 1,3- | 1,3- |
| | | | | di-t- | di-t- |
| | | | | BuPh | BuPh |
| 121105 | t-Bu | t-Bu | CN | 1,3- | 1,3- |
| | | | | di-t- | di-t- |
| | | | | BuPh | BuPh |
| 121106 | t-Bu | t-Bu | DPA | 1,3- | 1,3- |
| | | | | di-t- | di-t- |
| | | | | BuPh | BuPh |
| 121107 | t-Bu | t-Bu | 2- | 1,3- | 1,3- |
| | | | DBF | di-t- | di-t- |
| | | | | BuPh | BuPh |
| 121108 | t-Bu | t-Bu | 2- | 1,3- | 1,3- |
| | | | DBT | di-t- | dfr-t- |
| | | | | BuPh | BuPh |
| 121109 | t-Bu | t-Bu | 4-t- | 1,3- | 1,3- |
| | | | BuPh | di-t- | di-t- |
| | | | | BuPh | BuPh |
| 121110 | t-Bu | t-Bu | 1,3- | 1,3- | 1,3- |
| | | | di-t- | di-t- | di-t- |
| | | | BuPh | BuPh | BuPh |
| 121111 | t-Bu | t-Bu | 9-Cbz | 1,3- | 1,3- |
| | | | | di-t- | di-t- |
| | | | | BuPh | BuPh |
| 121112 | Ph | Ph | t-Bu | H | H |
| 121113 | Ph | Ph | CN | H | H |
| 121114 | Ph | Ph | DPA | H | H |
| 121115 | Ph | Ph | 2- | H | H |
| | | | DBF | | |
| 121116 | Ph | Ph | 2- | H | H |
| | | | DBT | | |
| 121117 | Ph | Ph | 4-t- | H | H |
| | | | BuPh | | |
| 121118 | Ph | Ph | 1,3- | H | H |
| | | | di-t- | | |
| | | | BuPh | | |
| 121119 | Ph | Ph | 9-Cbz | H | H |
| 121120 | Ph | Ph | t-Bu | t-Bu | t-Bu |
| 121121 | Ph | Ph | Ph | t-Bu | t-Bu |
| 121122 | Ph | Ph | CN | t-Bu | t-Bu |
| 121123 | Ph | Ph | DPA | t-Bu | t-Bu |
| 121124 | Ph | Ph | 2- | t-Bu | t-Bu |
| | | | DBF | | |
| 121125 | Ph | Ph | 2- | t-Bu | t-Bu |
| | | | DBT | | |
| 121126 | Ph | Ph | 4-t- | t-Bu | t-Bu |
| | | | BuPh | | |
| 121127 | Ph | Ph | 1,3- | t-Bu | t-Bu |
| | | | di-t- | | |
| | | | BuPh | | |
| 121128 | Ph | Ph | 9-Cbz | t-Bu | t-Bu |
| 121129 | Ph | Ph | Ph | Ph | Ph |
| 121130 | Ph | Ph | CN | Ph | Ph |
| 121131 | Ph | Ph | DPA | Ph | Ph |
| 121132 | Ph | Ph | 2- | Ph | Ph |
| | | | DBF | | |
| 121133 | Ph | Ph | 2- | Ph | Ph |
| | | | DBT | | |
| 121134 | Ph | Ph | 4-t- | Ph | Ph |
| | | | BuPh | | |
| 121135 | Ph | Ph | 1,3- | Ph | Ph |
| | | | di-t- | | |
| | | | BuPh | | |
| 121136 | Ph | Ph | 9-Cbz | Ph | Ph |
| 121137 | Ph | Ph | t-Bu | CN | CN |
| 121138 | Ph | Ph | Ph | CN | CN |
| 121139 | Ph | Ph | CN | CN | CN |
| 121140 | Ph | Ph | DPA | CN | CN |
| 121141 | Ph | Ph | 2- | CN | CN |
| | | | DBF | | |
| 121142 | Ph | Ph | 2- | CN | CN |
| | | | DBT | | |
| 121143 | Ph | Ph | 4-t- | CN | CN |
| | | | BuPh | | |
| 121144 | Ph | Ph | 1,3- | CN | CN |
| | | | di-t- | | |
| | | | BuPh | | |
| 121145 | Ph | Ph | 9-Cbz | CN | CN |
| 121146 | Ph | Ph | t-Bu | 9-Cbz | 9-Cbz |
| 121147 | Ph | Ph | Ph | 9-Cbz | 9-Cbz |
| 121148 | Ph | Ph | CN | 9-Cbz | 9-Cbz |
| 121149 | Ph | Ph | DPA | 9-Cbz | 9-Cbz |
| 121150 | Ph | Ph | 2- | 9-Cbz | 9-Cbz |
| | | | DBF | | |
| 121151 | Ph | Ph | 2- | 9-Cbz | 9-Cbz |
| | | | DBT | | |
| 121152 | Ph | Ph | 4-t- | 9-Cbz | 9-Cbz |
| | | | BuPh | | |
| 121153 | Ph | Ph | 1,3- | 9-Cbz | 9-Cbz |
| | | | di-t- | | |
| | | | BuPh | | |
| 121154 | Ph | Ph | 9-Cbz | 9-Cbz | 9-Cbz |
| 121155 | Ph | Ph | t-Bu | 4-t- | 4-t- |
| | | | | BuPh | BuPh |
| 121156 | Ph | Ph | Ph | 4-t- | 4-t- |
| | | | | BuPh | BuPh |
| 121157 | Ph | Ph | CN | 4-t- | 4-t- |
| | | | | BuPh | BuPh |
| 121158 | Ph | Ph | DPA | 4-t- | 4-t- |
| | | | | BuPh | BuPh |
| 121159 | Ph | Ph | 2- | 4-t- | 4-t- |
| | | | DBF | BuPh | BuPh |
| 121160 | Ph | Ph | 2- | 4-t- | 4-t- |
| | | | DBT | BuPh | BuPh |
| 121161 | Ph | Ph | 4-t- | 4-t- | 4-t- |
| | | | BuPh | BuPh | BuPh |
| 121162 | Ph | Ph | 1,3- | 4-t- | 4-t- |
| | | | di-t- | BuPh | BuPh |
| | | | BuPh | | |
| 121163 | Ph | Ph | 9-Cbz | 4-t- | 4-t- |
| | | | | BuPh | BuPh |
| 121164 | Ph | Ph | t-Bu | 1,3- | 1,3- |
| | | | | di-t- | di-t- |
| | | | | BuPh | BuPh |
| 121165 | Ph | Ph | Ph | 1,3- | 1,3- |
| | | | | di-t- | di-t- |
| | | | | BuPh | BuPh |
| 121166 | Ph | Ph | CN | 1,3- | 1,3- |
| | | | | di-t- | di-t- |
| | | | | BuPh | BuPh |
| 121167 | Ph | Ph | DPA | 1,3- | 1,3- |
| | | | | di-t- | di-t- |
| | | | | BuPh | BuPh |
| 121168 | Ph | Ph | 2- | 1,3- | 1,3- |
| | | | DBF | di-t- | di-t- |
| | | | | BuPh | BuPh |
| 121169 | Ph | Ph | 2- | 1,3- | 1,3- |
| | | | DBT | di-t- | di-t- |
| | | | | BuPh | BuPh |
| 121170 | Ph | Ph | 4-t- | 1,3- | 1,3- |
| | | | BuPh | di-t- | di-t- |
| | | | | BuPh | BuPh |
| 121171 | Ph | Ph | 1,3- | 1,3- | 1,3- |
| | | | di-t- | di-t- | di-t- |
| | | | BuPh | BuPh | BuPh |
| 121172 | Ph | Ph | 9-Cbz | 1,3- | 1,3- |
| | | | | di-t- | di-t- |
| | | | | BuPh | BuPh |
| 121173 | 9-Cbz | 9-Cbz | t-Bu | H | H |
| 121174 | 9-Cbz | 9-Cbz | Ph | H | H |
| 121175 | 9-Cbz | 9-Cbz | CN | H | H |
| 121176 | 9-Cbz | 9-Cbz | DPA | H | H |
| 121177 | 9-Cbz | 9-Cbz | 2- | H | H |
| | | | DBF | | |
| 121178 | 9-Cbz | 9-Cbz | 2- | H | H |
| | | | DBT | | |
| 121179 | 9-Cbz | 9-Cbz | 4-t- | H | H |
| | | | BuPh | | |
| 121180 | 9-Cbz | 9-Cbz | 1,3- | H | H |
| | | | di-t- | | |
| | | | BuPh | | |
| 121181 | 9-Cbz | 9-Cbz | t-Bu | t-Bu | t-Bu |
| 121182 | 9-Cbz | 9-Cbz | Ph | t-Bu | t-Bu |
| 121183 | 9-Cbz | 9-Cbz | CN | t-Bu | t-Bu |
| 121184 | 9-Cbz | 9-Cbz | DPA | t-Bu | t-Bu |
| 121185 | 9-Cbz | 9-Cbz | 2- | t-Bu | t-Bu |
| | | | DBF | | |
| 121186 | 9-Cbz | 9-Cbz | 2- | t-Bu | t-Bu |
| | | | DBT | | |
| 121187 | 9-Cbz | 9-Cbz | 4-t- | t-Bu | t-Bu |
| | | | BuPh | | |
| 121188 | 9-Cbz | 9-Cbz | 1,3- | t-Bu | t-Bu |
| | | | di-t- | | |
| | | | BuPh | | |
| 121189 | 9-Cbz | 9-Cbz | 9-Cbz | t-Bu | t-Bu |
| 121190 | 9-Cbz | 9-Cbz | t-Bu | Ph | Ph |
| 121191 | 9-Cbz | 9-Cbz | Ph | Ph | Ph |
| 121192 | 9-Cbz | 9-Cbz | CN | Ph | Ph |
| 121193 | 9-Cbz | 9-Cbz | DPA | Ph | Ph |
| 121194 | 9-Cbz | 9-Cbz | 2- | Ph | Ph |
| | | | DBF | | |
| 121195 | 9-Cbz | 9-Cbz | 2- | Ph | Ph |
| | | | DBT | | |
| 121196 | 9-Cbz | 9-Cbz | 4-t- | Ph | Ph |
| | | | BuPh | | |
| 121197 | 9-Cbz | 9-Cbz | 1,3- | Ph | Ph |
| | | | di-t- | | |
| | | | BuPh | | |
| 121198 | 9-Cbz | 9-Cbz | 9-Cbz | Ph | Ph |
| 121199 | 9-Cbz | 9-Cbz | t-Bu | CN | CN |
| 121200 | 9-Cbz | 9-Cbz | Ph | CN | CN |
| 121201 | 9-Cbz | 9-Cbz | CN | CN | CN |
| 121202 | 9-Cbz | 9-Cbz | DPA | CN | CN |
| 121203 | 9-Cbz | 9-Cbz | 2- | CN | CN |
| | | | DBF | | |
| 121204 | 9-Cbz | 9-Cbz | 2- | CN | CN |
| | | | DBT | | |
| 121205 | 9-Cbz | 9-Cbz | 4-t- | CN | CN |
| | | | BuPh | | |
| 121206 | 9-Cbz | 9-Cbz | 1,3- | CN | CN |
| | | | di-t- | | |
| | | | BuPh | | |
| 121207 | 9-Cbz | 9-Cbz | 9-Cbz | CN | CN |
| 121208 | 9-Cbz | 9-Cbz | t-Bu | 9-Cbz | 9-Cbz |
| 121209 | 9-Cbz | 9-Cbz | Ph | 9-Cbz | 9-Cbz |
| 121210 | 9-Cbz | 9-Cbz | CN | 9-Cbz | 9-Cbz |
| 121211 | 9-Cbz | 9-Cbz | DPA | 9-Cbz | 9-Cbz |
| 121212 | 9-Cbz | 9-Cbz | 2- | 9-Cbz | 9-Cbz |
| | | | DBF | | |
| 121213 | 9-Cbz | 9-Cbz | 2- | 9-Cbz | 9-Cbz |
| | | | DBT | | |
| 121214 | 9-Cbz | 9-Cbz | 4-t- | 9-Cbz | 9-Cbz |
| | | | BuPh | | |
| 121215 | 9-Cbz | 9-Cbz | 1,3- | 9-Cbz | 9-Cbz |
| | | | di-t- | | |
| | | | BuPh | | |
| 121216 | 9-Cbz | 9-Cbz | 9-Cbz | 9-Cbz | 9-Cbz |
| 121217 | 9-Cbz | 9-Cbz | t-Bu | 4-t- | 4-t- |
| | | | | BuPh | BuPh |
| 121218 | 9-Cbz | 9-Cbz | Ph | 4-t- | 4-t- |
| | | | | BuPh | BuPh |
| 121219 | 9-Cbz | 9-Cbz | CN | 4-t- | 4-t- |
| | | | | BuPh | BuPh |
| 121220 | 9-Cbz | 9-Cbz | DPA | 4-t- | 4-t- |
| | | | | BuPh | BuPh |
| 121221 | 9-Cbz | 9-Cbz | 2- | 4-t- | 4-t- |
| | | | DBF | BuPh | BuPh |
| 121222 | 9-Cbz | 9-Cbz | 2- | 4-t- | 4-t- |
| | | | DBT | BuPh | BuPh |
| 121223 | 9-Cbz | 9-Cbz | 4-t- | 4-t- | 4-t- |
| | | | BuPh | BuPh | BuPh |
| 121224 | 9-Cbz | 9-Cbz | 1,3- | 4-t- | 4-t- |
| | | | di-t- | BuPh | BuPh |
| | | | BuPh | | |
| 121225 | 9-Cbz | 9-Cbz | 9-Cbz | 4-t- | 4-t- |
| | | | | BuPh | BuPh |
| 121226 | 9-Cbz | 9-Cbz | t-Bu | 1,3- | 1,3- |
| | | | | di-t- | di-t- |
| | | | | BuPh | BuPh |
| 121227 | 9-Cbz | 9-Cbz | Ph | 1,3- | 1,3- |
| | | | | di-t- | di-t- |
| | | | | BuPh | BuPh |
| 121228 | 9-Cbz | 9-Cbz | CN | 1,3- | 1,3- |
| | | | | di-t- | di-t- |
| | | | | BuPh | BuPh |
| 121229 | 9-Cbz | 9-Cbz | DPA | 1,3- | 1,3- |
| | | | | di-t- | di-t- |
| | | | | BuPh | BuPh |
| 121230 | 9-Cbz | 9-Cbz | 2- | 1,3- | 1,3- |
| | | | DBF | di-t- | di-t- |
| | | | | BuPh | BuPh |
| 121231 | 9-Cbz | 9-Cbz | 2- | 1,3- | 1,3- |
| | | | DBT | di-t- | di-t- |
| | | | | BuPh | BuPh |
| 121232 | 9-Cbz | 9-Cbz | 4-t- | 1,3- | 1,3- |
| | | | BuPh | di-t- | di-t- |
| | | | | BuPh | BuPh |
| 121233 | 9-Cbz | 9-Cbz | 1,3- | 1,3- | 1,3- |
| | | | di-t- | di-t- | di-t- |
| | | | BuPh | BuPh | BuPh |
| 121234 | 9-Cbz | 9-Cbz | 9-Cbz | 1,3- | 1,3- |
| | | | | di-t- | di-t- |
| | | | | BuPh | BuPh |
| 121235 | CN | CN | t-Bu | H | H |
| 121236 | CN | CN | Ph | H | H |
| 121237 | CN | CN | DPA | H | H |
| 121238 | CN | CN | 2- | H | H |
| | | | DBF | | |
| 121239 | CN | CN | 2- | H | H |
| | | | DBT | | |
| 121240 | CN | CN | 4-t- | H | H |
| | | | BuPh | | |
| 121241 | CN | CN | 1,3- | H | H |
| | | | di-t- | | |
| | | | BuPh | | |
| 121242 | CN | CN | 9-Cbz | H | H |
| 121243 | CN | CN | t-Bu | t-Bu | t-Bu |
| 121244 | CN | CN | Ph | t-Bu | t-Bu |
| 121245 | CN | CN | CN | t-Bu | t-Bu |
| 121246 | CN | CN | DPA | t-Bu | t-Bu |
| 121247 | CN | CN | 2- | t-Bu | t-Bu |
| | | | DBF | | |
| 121248 | CN | CN | 2- | t-Bu | t-Bu |
| | | | DBT | | |
| 121249 | CN | CN | 4-t- | t-Bu | t-Bu |
| | | | BuPh | | |
| 121250 | CN | CN | 1,3- | t-Bu | t-Bu |
| | | | di-t- | | |
| | | | BuPh | | |
| 121251 | CN | CN | 9-Cbz | t-Bu | t-Bu |
| 121252 | CN | CN | t-Bu | Ph | Ph |
| 121253 | CN | CN | Ph | Ph | Ph |
| 121254 | CN | CN | CN | Ph | Ph |
| 121255 | CN | CN | DPA | Ph | Ph |
| 121256 | CN | CN | 2- | Ph | Ph |
| | | | DBF | | |
| 121257 | CN | CN | 2- | Ph | Ph |
| | | | DBT | | |
| 121258 | CN | CN | 4-t- | Ph | Ph |
| | | | BuPh | | |
| 121259 | CN | CN | 1,3- | Ph | Ph |
| | | | di-t- | | |
| | | | BuPh | | |
| 121260 | CN | CN | 9-Cbz | Ph | Ph |
| 121261 | CN | CN | t-Bu | CN | CN |
| 121262 | CN | CN | Ph | CN | CN |
| 121263 | CN | CN | CN | CN | CN |
| 121264 | CN | CN | DPA | CN | CN |
| 121265 | CN | CN | 2- | CN | CN |
| | | | DBF | | |
| 121266 | CN | CN | 2- | CN | CN |
| | | | DBT | | |
| 121267 | CN | CN | 4-t- | CN | CN |
| | | | BuPh | | |
| 121268 | CN | CN | 1,3- | CN | CN |
| | | | di-t- | | |
| | | | BuPh | | |
| 121269 | CN | CN | 9-Cbz | CN | CN |
| 121270 | CN | CN | t-Bu | 9-Cbz | 9-Cbz |
| 121271 | CN | CN | Ph | 9-Cbz | 9-Cbz |
| 121272 | CN | CN | CN | 9-Cbz | 9-Cbz |
| 121273 | CN | CN | DPA | 9-Cbz | 9-Cbz |
| 121274 | CN | CN | 2- | 9-Cbz | 9-Cbz |
| | | | DBF | | |
| 121275 | CN | CN | 2- | 9-Cbz | 9-Cbz |
| | | | DBT | | |
| 121276 | CN | CN | 4-t- | 9-Cbz | 9-Cbz |
| | | | BuPh | | |
| 121277 | CN | CN | 1,3- | 9-Cbz | 9-Cbz |
| | | | di-t- | | |
| | | | BuPh | | |
| 121278 | CN | CN | 9-Cbz | 9-Cbz | 9-Cbz |
| 121279 | CN | CN | t-Bu | 4-t- | 4-t- |
| | | | | BuPh | BuPh |
| 121280 | CN | CN | Ph | 4-t- | 4-t- |
| | | | | BuPh | BuPh |
| 121281 | CN | CN | CN | 4-t- | 4-t- |
| | | | | BuPh | BuPh |
| 121282 | CN | CN | DPA | 4-t- | 4-t- |
| | | | | BuPh | BuPh |
| 121283 | CN | CN | 2- | 4-t- | 4-t- |
| | | | DBF | BuPh | BuPh |
| 121284 | CN | CN | 2- | 4-t- | 4-t- |
| | | | DBT | BuPh | BuPh |
| 121285 | CN | CN | 4-t- | 4-t- | 4-t- |
| | | | BuPh | BuPh | BuPh |
| 121286 | CN | CN | 1,3- | 4-t- | 4-t- |
| | | | di-t- | BuPh | BuPh |
| | | | BuPh | | |
| 121287 | CN | CN | 9-Cbz | 4-t- | 4-t- |
| | | | | BuPh | BuPh |
| 121288 | CN | CN | t-Bu | 1,3- | 1,3- |
| | | | | di-t- | di-t- |
| | | | | BuPh | BuPh |
| 121289 | CN | CN | Ph | 1,3- | 1,3- |
| | | | | di-t- | di-t- |
| | | | | BuPh | BuPh |
| 121290 | CN | CN | CN | 1,3- | 1,3- |
| | | | | di-t- | di-t- |
| | | | | BuPh | BuPh |
| 121291 | CN | CN | DPA | 1,3- | 1,3- |
| | | | | di-t- | di-t- |
| | | | | BuPh | BuPh |
| 121292 | CN | CN | 2- | 1,3- | 1,3- |
| | | | DBF | di-t- | di-t- |
| | | | | BuPh | BuPh |
| 121293 | CN | CN | 2- | 1,3- | 1,3- |
| | | | DBT | di-t- | di-t- |
| | | | | BuPh | BuPh |
| 121294 | CN | CN | 4-t- | 1,3- | 1,3- |
| | | | BuPh | di-t- | di-t- |
| | | | | BuPh | BuPh |
| 121295 | CN | CN | 1,3- | 1,3- | 1,3- |
| | | | di-t- | di-t- | di-t- |
| | | | BuPh | BuPh | BuPh |
| 121296 | CN | CN | 9-Cbz | 1,3- | 1,3- |
| | | | | di-t- | di-t- |
| | | | | BuPh | BuPh |
| 121297 | 4-t- | 4-t- | t-Bu | H | H |
| | BuPh | BuPh | | | |
| 121298 | 4-t- | 4-t- | Ph | H | H |
| | BuPh | BuPh | | | |
| 121299 | 4-t- | 4-t- | CN | H | H |
| | BuPh | BuPh | | | |
| 121300 | 4-t- | 4-t- | DPA | H | H |
| | BuPh | BuPh | | | |
| 121301 | 4-t- | 4-t- | 2- | H | H |
| | BuPh | BuPh | DBF | | |
| 121302 | 4-t- | 4-t- | 2- | H | H |
| | BuPh | BuPh | DBT | | |
| 121303 | 4-t- | 4-t- | 1,3- | H | H |
| | BuPh | BuPh | di-t- | | |
| | | | BuPh | | |
| 121304 | 4-t- | 4-t- | 9-Cbz | H | H |
| | BuPh | BuPh | | | |
| 121305 | 4-t- | 4-t- | t-Bu | t-Bu | t-Bu |
| | BuPh | BuPh | | | |
| 121306 | 4-t- | 4-t- | Ph | t-Bu | t-Bu |
| | BuPh | BuPh | | | |
| 121307 | 4-t- | 4-t- | CN | t-Bu | t-Bu |
| | BuPh | BuPh | | | |
| 121308 | 4-t- | 4-t- | DPA | t-Bu | t-Bu |
| | BuPh | BuPh | | | |
| 121309 | 4-t- | 4-t- | 2- | t-Bu | t-Bu |
| | BuPh | BuPh | DBF | | |
| 121310 | 4-t- | 4-t- | 2- | t-Bu | t-Bu |
| | BuPh | BuPh | DBT | | |
| 121311 | 4-t- | 4-t- | 4-t- | t-Bu | t-Bu |
| | BuPh | BuPh | BuPh | | |
| 121312 | 4-t- | 4-t- | 1,3- | t-Bu | t-Bu |
| | BuPh | BuPh | di-t- | | |
| | | | BuPh | | |
| 121313 | 4-t- | 4-t- | 9-Cbz | t-Bu | t-Bu |
| | BuPh | BuPh | | | |
| 121314 | 4-t- | 4-t- | t-Bu | Ph | Ph |
| | BuPh | BuPh | | | |
| 121315 | 4-t- | 4-t- | Ph | Ph | Ph |
| | BuPh | BuPh | | | |
| 121316 | 4-t- | 4-t- | CN | Ph | Ph |
| | BuPh | BuPh | | | |
| 121317 | 4-t- | 4-t- | DPA | Ph | Ph |
| | BuPh | BuPh | | | |
| 121318 | 4-t- | 4-t- | 2- | Ph | Ph |
| | BuPh | BuPh | DBF | | |
| 121319 | 4-t- | 4-t- | 2- | Ph | Ph |
| | BuPh | BuPh | DBT | | |
| 121320 | 4-t- | 4-t- | 4-t- | Ph | Ph |
| | BuPh | BuPh | BuPh | | |
| 121321 | 4-t- | 4-t- | 1,3- | Ph | Ph |
| | BuPh | BuPh | di-t- | | |
| | | | BuPh | | |
| 121322 | 4-t- | 4-t- | 9-Cbz | Ph | Ph |
| | BuPh | BuPh | | | |
| 121323 | 4-t- | 4-t- | t-Bu | CN | CN |
| | BuPh | BuPh | | | |
| 121324 | 4-t- | 4-t- | Ph | CN | CN |
| | BuPh | BuPh | | | |
| 121325 | 4-t- | 4-t- | CN | CN | CN |
| | BuPh | BuPh | | | |
| 121326 | 4-t- | 4-t- | DPA | CN | CN |
| | BuPh | BuPh | | | |
| 121327 | 4-t- | 4-t- | 2- | CN | CN |
| | BuPh | BuPh | DBF | | |
| 121328 | 4-t- | 4-t- | 2- | CN | CN |
| | BuPh | BuPh | DBT | | |
| 121329 | 4-t- | 4-t- | 4-t- | CN | CN |
| | BuPh | BuPh | BuPh | | |
| 121330 | 4-t- | 4-t- | 1,3- | CN | CN |
| | BuPh | BuPh | di-t- | | |
| | | | BuPh | | |
| 121331 | 4-t- | 4-t- | 9-Cbz | CN | CN |
| | BuPh | BuPh | | | |
| 121332 | 4-t- | 4-t- | t-Bu | 9-Cbz | 9-Cbz |
| | BuPh | BuPh | | | |
| 121333 | 4-t- | 4-t- | Ph | 9-Cbz | 9-Cbz |
| | BuPh | BuPh | | | |
| 121334 | 4-t- | 4-t- | CN | 9-Cbz | 9-Cbz |
| | BuPh | BuPh | | | |
| 121335 | 4-t- | 4-t- | DPA | 9-Cbz | 9-Cbz |
| | BuPh | BuPh | | | |
| 121336 | 4-t- | 4-t- | 2- | 9-Cbz | 9-Cbz |
| | BuPh | BuPh | DBF | | |
| 121337 | 4-t- | 4-t- | 2- | 9-Cbz | 9-Cbz |
| | BuPh | BuPh | DBT | | |
| 121338 | 4-t- | 4-t- | 4-t- | 9-Cbz | 9-Cbz |
| | BuPh | BuPh | BuPh | | |
| 121339 | 4-t- | 4-t- | 1,3- | 9-Cbz | 9-Cbz |
| | BuPh | BuPh | di-t- | | |
| | | | BuPh | | |
| 121340 | 4-t- | 4-t- | 9-Cbz | 9-Cbz | 9-Cbz |
| | BuPh | BuPh | | | |
| 121341 | 4-t- | 4-t- | t-Bu | 4-t- | 4-t- |
| | BuPh | BuPh | | BuPh | BuPh |
| 121342 | 4-t- | 4-t- | Ph | 4-t- | 4-t- |
| | BuPh | BuPh | | BuPh | BuPh |
| 121343 | 4-t- | 4-t- | CN | 4-t- | 4-t- |
| | BuPh | BuPh | | BuPh | BuPh |
| 121344 | 4-t- | 4-t- | DPA | 4-t- | 4-t- |
| | BuPh | BuPh | | BuPh | BuPh |
| 121345 | 4-t- | 4-t- | 2- | 4-t- | 4-t- |
| | BuPh | BuPh | DBF | BuPh | BuPh |
| 121346 | 4-t- | 4-t- | 2- | 4-t- | 4-t- |
| | BuPh | BuPh | DBT | BuPh | BuPh |
| 121347 | 4-t- | 4-t- | 4-t- | 4-t- | 4-t- |
| | BuPh | BuPh | BuPh | BuPh | BuPh |
| 121348 | 4-t- | 4-t- | 1,3- | 4-t- | 4-t- |
| | BuPh | BuPh | di-t- | BuPh | BuPh |
| | | | BuPh | | |
| 121349 | 4-t- | 4-t- | 9-Cbz | 4-t- | 4-t- |
| | BuPh | BuPh | | BuPh | BuPh |
| 121350 | 4-t- | 4-t- | t-Bu | 1,3- | 1,3- |
| | BuPh | BuPh | | di-t- | di-t- |
| | | | | BuPh | BuPh |
| 121351 | 4-t- | 4-t- | Ph | 1,3- | 1,3- |
| | BuPh | BuPh | | di-t- | di-t- |
| | | | | BuPh | BuPh |
| 121352 | 4-t- | 4-t- | CN | 1,3- | 1,3- |
| | BuPh | BuPh | | di-t- | di-t- |
| | | | | BuPh | BuPh |
| 121353 | 4-t- | 4-t- | DPA | 1,3- | 1,3- |
| | BuPh | BuPh | | di-t- | di-t- |
| | | | | BuPh | BuPh |
| 121354 | 4-t- | 4-t- | 2- | 1,3- | 1,3- |
| | BuPh | BuPh | DBF | di-t- | di-t- |
| | | | | BuPh | BuPh |
| 121355 | 4-t- | 4-t- | 2- | 1,3- | 1,3- |
| | BuPh | BuPh | DBT | di-t- | di-t- |
| | | | | BuPh | BuPh |
| 121356 | 4-t- | 4-t- | 4-t- | 1,3- | 1,3- |
| | BuPh | BuPh | BuPh | di-t- | di-t- |
| | | | | BuPh | BuPh |
| 121357 | 4-t- | 4-t- | 1,3- | 1,3- | 1,3- |
| | BuPh | BuPh | di-t- | di-t- | di-t- |
| | | | BuPh | BuPh | BuPh |
| 121358 | 4-t- | 4-t- | 9-Cbz | 1,3- | 1,3- |
| | BuPh | BuPh | | di-t- | di-t- |
| | | | | BuPh | BuPh |
| 121359 | 1,3- | 1,3- | t-Bu | H | H |
| | di-t- | di-t- | | | |
| | BuPh | BuPh | | | |
| 121360 | 1,3- | 1,3- | Ph | H | H |
| | di-t- | di-t- | | | |
| | BuPh | BuPh | | | |
| 121361 | 1,3- | 1,3- | CN | H | H |
| | di-t- | di-t- | | | |
| | BuPh | BuPh | | | |
| 121362 | 1,3- | 1,3- | DPA | H | H |
| | di-t- | di-t- | | | |
| | BuPh | BuPh | | | |
| 121363 | 1,3- | 1,3- | 2- | H | H |
| | di-t- | di-t- | DBF | | |
| | BuPh | BuPh | | | |
| 121364 | 1,3- | 1,3- | 2- | H | H |
| | di-t- | di-t- | DBT | | |
| | BuPh | BuPh | | | |
| 121365 | 1,3- | 1,3- | 4-t- | H | H |
| | di-t- | di-t- | BuPh | | |
| | BuPh | BuPh | | | |
| 121366 | 1,3- | 1,3- | 9-Cbz | H | H |
| | di-t- | di-t- | | | |
| | BuPh | BuPh | | | |
| 121367 | 1,3- | 1,3- | t-Bu | t-Bu | t-Bu |
| | di-t- | di-t- | | | |
| | BuPh | BuPh | | | |
| 121368 | 1,3- | 1,3- | Ph | t-Bu | t-Bu |
| | di-t- | di-t- | | | |
| | BuPh | BuPh | | | |
| 121369 | 1,3- | 1,3- | CN | t-Bu | t-Bu |
| | di-t- | di-t- | | | |
| | BuPh | BuPh | | | |
| 121370 | 1,3- | 1,3- | DPA | t-Bu | t-Bu |
| | di-t- | di-t- | | | |
| | BuPh | BuPh | | | |
| 121371 | 1,3- | 1,3- | 2- | t-Bu | t-Bu |
| | di-t- | di-t- | DBF | | |
| | BuPh | BuPh | | | |
| 121372 | 1,3- | 1,3- | 2- | t-Bu | t-Bu |
| | di-t- | di-t- | DBT | | |
| | BuPh | BuPh | | | |
| 121373 | 1,3- | 1,3- | 4-t- | t-Bu | t-Bu |
| | di-t- | di-t- | BuPh | | |
| | BuPh | BuPh | | | |
| 121374 | 1,3- | 1,3- | 1,3- | t-Bu | t-Bu |
| | di-t- | di-t- | di-t- | | |
| | BuPh | BuPh | BuPh | | |
| 121375 | 1,3- | 1,3- | 9-Cbz | t-Bu | t-Bu |
| | di-t- | di-t- | | | |
| | BuPh | BuPh | | | |
| 121376 | 1,3- | 1,3- | t-Bu | Ph | Ph |
| | di-t- | di-t- | | | |
| | BuPh | BuPh | | | |
| 121377 | 1,3- | 1,3- | Ph | Ph | Ph |
| | di-t- | di-t- | | | |
| | BuPh | BuPh | | | |
| 121378 | 1,3- | 1,3- | CN | Ph | Ph |
| | di-t- | di-t- | | | |
| | BuPh | BuPh | | | |
| 121379 | 1,3- | 1,3- | DPA | Ph | Ph |
| | di-t- | di-t- | | | |
| | BuPh | BuPh | | | |
| 121380 | 1,3- | 1,3- | 2- | Ph | Ph |
| | di-t- | di-t- | DBF | | |
| | BuPh | BuPh | | | |
| 121381 | 1,3- | 1,3- | 2- | Ph | Ph |
| | di-t- | di-t- | DBT | | |
| | BuPh | BuPh | | | |
| 121382 | 1,3- | 1,3- | 4-t- | Ph | Ph |
| | di-t- | di-t- | BuPh | | |
| | BuPh | BuPh | | | |
| 121383 | 1,3- | 1,3- | 1,3- | Ph | Ph |
| | di-t- | di-t- | di-t- | | |
| | BuPh | BuPh | BuPh | | |
| 121384 | 1,3- | 1,3- | 9-Cbz | Ph | Ph |
| | di-t- | di-t- | | | |
| | BuPh | BuPh | | | |
| 121385 | 1,3- | 1,3- | t-Bu | CN | CN |
| | di-t- | di-t- | | | |
| | BuPh | BuPh | | | |
| 121386 | 1,3- | 1,3- | Ph | CN | CN |
| | di-t- | di-t- | | | |
| | BuPh | BuPh | | | |
| 121387 | 1,3- | 1,3- | CN | CN | CN |
| | di-t- | di-t- | | | |
| | BuPh | BuPh | | | |
| 121388 | 1,3- | 1,3- | DPA | CN | CN |
| | di-t- | di-t- | | | |
| | BuPh | BuPh | | | |
| 121389 | 1,3- | 1,3- | 2- | CN | CN |
| | di-t- | di-t- | DBF | | |
| | BuPh | BuPh | | | |
| 121390 | 1,3- | 1,3- | 2- | CN | CN |
| | di-t- | di-t- | DBT | | |
| | BuPh | BuPh | | | |
| 121391 | 1,3- | 1,3- | 4-t- | CN | CN |
| | di-t- | di-t- | BuPh | | |
| | BuPh | BuPh | | | |
| 121392 | 1,3- | 1,3- | 1,3- | CN | CN |
| | di-t- | di-t- | di-t- | | |
| | BuPh | BuPh | BuPh | | |
| 121393 | 1,3- | 1,3- | 9-Cbz | CN | CN |
| | di-t- | di-t- | | | |
| | BuPh | BuPh | | | |
| 121394 | 1,3- | 1,3- | t-Bu | 9-Cbz | 9-Cbz |
| | di-t- | di-t- | | | |
| | BuPh | BuPh | | | |
| 121395 | 1,3- | 1,3- | Ph | 9-Cbz | 9-Cbz |
| | di-t- | di-t- | | | |
| | BuPh | BuPh | | | |
| 121396 | 1,3- | 1,3- | CN | 9-Cbz | 9-Cbz |
| | di-t- | di-t- | | | |
| | BuPh | BuPh | | | |
| 121397 | 1,3- | 1,3- | DPA | 9-Cbz | 9-Cbz |
| | di-t- | di-t- | | | |
| | BuPh | BuPh | | | |
| 121398 | 1,3- | 1,3- | 2- | 9-Cbz | 9-Cbz |
| | di-t- | di-t- | DBF | | |
| | BuPh | BuPh | | | |
| 121399 | 1,3- | 1,3- | 2- | 9-Cbz | 9-Cbz |
| | di-t- | di-t- | DBT | | |
| | BuPh | BuPh | | | |
| 121400 | 1,3- | 1,3- | 4-t- | 9-Cbz | 9-Cbz |
| | di-t- | di-t- | BuPh | | |
| | BuPh | BuPh | | | |
| 121401 | 1,3- | 1,3- | 1,3- | 9-Cbz | 9-Cbz |
| | di-t- | di-t- | di-t- | | |
| | BuPh | BuPh | BuPh | | |
| 121402 | 1,3- | 1,3- | 9-Cbz | 9-Cbz | 9-Cbz |
| | di-t- | di-t- | | | |
| | BuPh | BuPh | | | |
| 121403 | 1,3- | 1,3- | t-Bu | 4-t- | 4-t- |
| | di-t- | di-t- | | BuPh | BuPh |
| | BuPh | BuPh | | | |
| 121404 | 1,3- | 1,3- | Ph | 4-t- | 4-t- |
| | di-t- | di-t- | | BuPh | BuPh |
| | BuPh | BuPh | | | |
| 121405 | 1,3- | 1,3- | CN | 4-t- | 4-t- |
| | di-t- | di-t- | | BuPh | BuPh |
| | BuPh | BuPh | | | |
| 121406 | 1,3- | 1,3- | DPA | 4-t- | 4-t- |
| | di-t- | di-t- | | BuPh | BuPh |
| | BuPh | BuPh | | | |
| 121407 | 1,3- | 1,3- | 2- | 4-t- | 4-t- |
| | di-t- | di-t- | DBF | BuPh | BuPh |
| | BuPh | BuPh | | | |
| 121408 | 1,3- | 1,3- | 2- | 4-t- | 4-t- |
| | di-t- | di-t- | DBT | BuPh | BuPh |
| | BuPh | BuPh | | | |
| 121409 | 1,3- | 1,3- | 4-t- | 4-t- | 4-t- |
| | di-t- | di-t- | BuPh | BuPh | BuPh |
| | BuPh | BuPh | | | |
| 121410 | 1,3- | 1,3- | 1,3- | 4-t- | 4-t- |
| | di-t- | di-t- | di-t- | BuPh | BuPh |
| | BuPh | BuPh | BuPh | | |
| 121411 | 1,3- | 1,3- | 9-Cbz | 4-t- | 4-t- |
| | di-t- | di-t- | | BuPh | BuPh |
| | BuPh | BuPh | | | |
| 121412 | 1,3- | 1,3- | t-Bu | 1,3- | 1,3- |
| | di-t- | di-t- | | di-t- | di-t- |
| | BuPh | BuPh | | BuPh | BuPh |
| 121413 | 1,3- | 1,3- | Ph | 1,3- | 1,3- |
| | di-t- | di-t- | | di-t- | di-t- |
| | BuPh | BuPh | | BuPh | BuPh |
| 121414 | 1,3- | 1,3- | CN | 1,3- | 1,3- |
| | di-t- | di-t- | | di-t- | di-t- |
| | BuPh | BuPh | | BuPh | BuPh |
| 121415 | 1,3- | 1,3- | DPA | 1,3- | 1,3- |
| | di-t- | di-t- | | di-t- | di-t- |
| | BuPh | BuPh | | BuPh | BuPh |
| 121416 | 1,3- | 1,3- | 2- | 1,3- | 1,3- |
| | di-t- | di-t- | DBF | di-t- | di-t- |
| | BuPh | BuPh | | BuPh | BuPh |
| 121417 | 1,3- | 1,3- | 2- | 1,3- | 1,3- |
| | di-t- | di-t- | DBT | di-t- | di-t- |
| | BuPh | BuPh | | BuPh | BuPh |
| 121418 | 1,3- | 1,3- | 4-t- | 1,3- | 1,3- |
| | di-t- | di-t- | BuPh | di-t- | di-t- |
| | BuPh | BuPh | | BuPh | BuPh |
| 121419 | 1,3- | 1,3- | 1,3- | 1,3- | 1,3- |
| | di-t- | di-t- | di-t- | di-t- | di-t- |
| | BuPh | BuPh | BuPh | BuPh | BuPh |
| 121420 | 1,3- | 1,3- | 9-Cbz | 1,3- | 1,3- |
| | di-t- | di-t- | | di-t- | di-t- |
| | BuPh | BuPh | | BuPh | BuPh |

### Group HC4

The heterocyclic compound may be represented by Formula 12-2, and substituents may be defined as shown in Table 4.

TABLE 4

| Compound | | | | | |
|---|---|---|---|---|---|
| No. | R_{11b} | R_{12c} | R_{14b} | R_{18c} | R_{19b} |
| 122001 | t-Bu | t-Bu | t-Bu | t-Bu | t-Bu |
| 122002 | Ph | Ph | t-Bu | Ph | Ph |
| 122003 | t-Bu | t-Bu | H | H | H |
| 122004 | H | H | t-Bu | H | H |
| 122005 | H | H | H | t-Bu | t-Bu |
| 122006 | t-Bu | t-Bu | t-Bu | H | H |
| 122007 | H | H | t-Bu | t-Bu | t-Bu |
| 122008 | t-Bu | t-Bu | H | t-Bu | t-Bu |
| 122009 | Ph | Ph | H | H | H |
| 122010 | H | H | Ph | H | H |
| 122011 | H | H | H | Ph | Ph |
| 122012 | Ph | Ph | Ph | H | H |
| 122013 | H | H | Ph | Ph | Ph |
| 122014 | Ph | Ph | H | Ph | Ph |
| 122015 | CN | CN | H | H | H |
| 122016 | H | H | CN | H | H |
| 122017 | H | H | H | CN | CN |
| 122018 | CN | CN | CN | H | H |
| 122019 | H | H | CN | CN | CN |
| 122020 | CN | CN | H | CN | CN |
| 122021 | 9-Cbz | 9-Cbz | H | H | H |
| 122022 | H | H | 9-Cbz | H | H |
| 122023 | H | H | H | 9-Cbz | 9-Cbz |
| 122024 | 9-Cbz | 9-Cbz | 9-Cbz | H | H |
| 122025 | H | H | 9-Cbz | 9-Cbz | 9-Cbz |
| 122026 | 9-Cbz | 9-Cbz | H | 9-Cbz | 9-Cbz |
| 122027 | Me | Me | H | H | H |
| 122028 | H | H | Me | H | H |
| 122029 | H | H | H | Me | Me |
| 122030 | Me | Me | Me | H | H |
| 122031 | H | H | Me | Me | Me |
| 122032 | Me | Me | H | Me | Me |
| 122033 | CD₃ | CD3 | H | H | H |
| 122034 | H | H | CD3 | H | H |
| 122035 | H | H | H | CD3 | CD3 |
| 122036 | CD3 | CD3 | CD3 | H | H |
| 122037 | H | H | CD3 | CD3 | CD3 |
| 122038 | CD3 | CD3 | H | CD3 | CD3 |
| 122039 | 4-t- | 4-t- | H | H | H |
| | BuPh | BuPh | | | |
| 122040 | H | H | 4-t- | H | H |
| | | | BuPh | | |
| 122041 | H | H | H | 4-t- | 4-t- |
| | | | | BuPh | BuPh |
| 122042 | 4-t- | 4-t- | 4-t- | H | H |
| | BuPh | BuPh | BuPh | | |
| 122043 | H | H | 4-t- | 4-t- | 4-t- |
| | | | BuPh | BuPh | BuPh |
| 122044 | 4-t- | 4-t- | H | 4-t- | 4-t- |
| | BuPh | BuPh | | BuPh | BuPh |
| 122045 | 1,3- | 1,3- | H | H | H |
| | di-t- | di-t- | | | |
| | BuPh | BuPh | | | |
| 122046 | H | H | 1,3- | H | H |
| | | | di-t- | | |
| | | | BuPh | | |
| 122047 | H | H | H | 1,3- | 1,3- |
| | | | | di-t- | di-t- |
| | | | | BuPh | BuPh |
| 122048 | 1,3- | 1,3- | 1,3- | H | H |
| | di-t- | di-t- | di-t- | | |
| | BuPh | BuPh | BuPh | | |
| 122049 | H | H | 1,3- | 1,3- | 1,3- |
| | | | di-t- | di-t- | di-t- |
| | | | BuPh | BuPh | BuPh |
| 122050 | 1,3- | 1,3- | H | 1,3- | 1,3- |
| | di-t- | di-t- | | di-t- | di-t- |
| | BuPh | BuPh | | BuPh | BuPh |
| 122051 | t-Bu | t-Bu | Ph | H | H |
| 122052 | t-Bu | t-Bu | CN | H | H |
| 122053 | t-Bu | t-Bu | DPA | H | H |
| 122054 | t-Bu | t-Bu | 2- | H | H |
| | | | DBF | | |
| 122055 | t-Bu | t-Bu | 2- | H | H |
| | | | DBT | | |
| 122056 | t-Bu | t-Bu | 4-t- | H | H |
| | | | BuPh | | |
| 122057 | t-Bu | t-Bu | 1,3- | H | H |
| | | | di-t- | | |
| | | | BuPh | | |
| 122058 | t-Bu | t-Bu | 9-Cbz | H | H |
| 122059 | t-Bu | t-Bu | Ph | t-Bu | t-Bu |
| 122060 | t-Bu | t-Bu | CN | t-Bu | t-Bu |
| 122061 | t-Bu | t-Bu | DPA | t-Bu | t-Bu |
| 122062 | t-Bu | t-Bu | 2- | t-Bu | t-Bu |
| | | | DBF | | |
| 122063 | t-Bu | t-Bu | 2- | t-Bu | t-Bu |
| | | | DBT | | |
| 122064 | t-Bu | t-Bu | 4-t- | t-Bu | t-Bu |
| | | | BuPh | | |
| 122065 | t-Bu | t-Bu | 1,3- | t-Bu | t-Bu |
| | | | di-t- | | |
| | | | BuPh | | |
| 122066 | t-Bu | t-Bu | 9-Cbz | t-Bu | t-Bu |
| 122067 | t-Bu | t-Bu | t-Bu | Ph | Ph |
| 122068 | t-Bu | t-Bu | Ph | Ph | Ph |
| 122069 | t-Bu | t-Bu | CN | Ph | Ph |
| 122070 | t-Bu | t-Bu | DPA | Ph | Ph |
| 122071 | t-Bu | t-Bu | 2- | Ph | Ph |
| | | | DBF | | |
| 122072 | t-Bu | t-Bu | 2- | Ph | Ph |
| | | | DBT | | |
| 122073 | t-Bu | t-Bu | 4-t- | Ph | Ph |
| | | | BuPh | | |
| 122074 | t-Bu | t-Bu | 1,3- | Ph | Ph |
| | | | di-t- | | |
| | | | BuPh | | |
| 122075 | t-Bu | t-Bu | 9-Cbz | Ph | Ph |
| 122076 | t-Bu | t-Bu | t-Bu | CN | CN |
| 122077 | t-Bu | t-Bu | Ph | CN | CN |
| 122078 | t-Bu | t-Bu | CN | CN | CN |
| 122079 | t-Bu | t-Bu | DPA | CN | CN |
| 122080 | t-Bu | t-Bu | 2- | CN | CN |
| | | | DBF | | |
| 122081 | t-Bu | t-Bu | 2- | CN | CN |
| | | | DBT | | |
| 122082 | t-Bu | t-Bu | 4-t- | CN | CN |
| | | | BuPh | | |
| 122083 | t-Bu | t-Bu | 1,3- | CN | CN |
| | | | di-t- | | |
| | | | BuPh | | |
| 122084 | t-Bu | t-Bu | 9-Cbz | CN | CN |
| 122085 | t-Bu | t-Bu | t-Bu | 9-Cbz | 9-Cbz |
| 122086 | t-Bu | t-Bu | Ph | 9-Cbz | 9-Cbz |
| 122087 | t-Bu | t-Bu | CN | 9-Cbz | 9-Cbz |
| 122088 | t-Bu | t-Bu | DPA | 9-Cbz | 9-Cbz |
| 122089 | t-Bu | t-Bu | 2- | 9-Cbz | 9-Cbz |
| | | | DBF | | |
| 122090 | t-Bu | t-Bu | 2- | 9-Cbz | 9-Cbz |
| | | | DBT | | |
| 122091 | t-Bu | t-Bu | 4-t- | 9-Cbz | 9-Cbz |
| | | | BuPh | | |
| 122092 | t-Bu | t-Bu | 1,3- | 9-Cbz | 9-Cbz |
| | | | di-t- | | |
| | | | BuPh | | |
| 122093 | t-Bu | t-Bu | 9-Cbz | 9-Cbz | 9-Cbz |
| 122094 | t-Bu | t-Bu | t-Bu | 4-t- | 4-t- |
| | | | | BuPh | BuPh |
| 122095 | t-Bu | t-Bu | Ph | 4-t- | 4-t- |
| | | | | BuPh | BuPh |
| 122096 | t-Bu | t-Bu | CN | 4-t- | 4-t- |
| | | | | BuPh | BuPh |
| 122097 | t-Bu | t-Bu | DPA | 4-t- | 4-t- |
| | | | | BuPh | BuPh |
| 122098 | t-Bu | t-Bu | 2- | 4-t- | 4-t- |
| | | | DBF | BuPh | BuPh |
| 122099 | t-Bu | t-Bu | 2- | 4-t- | 4-t- |
| | | | DBT | BuPh | BuPh |
| 122100 | t-Bu | t-Bu | 4-t- | 4-t- | 4-t- |
| | | | BuPh | BuPh | BuPh |
| 122101 | t-Bu | t-Bu | 1,3- | 4-t- | 4-t- |
| | | | di-t- | BuPh | BuPh |
| | | | BuPh | | |
| 122102 | t-Bu | t-Bu | 9-Cbz | 4-t- | 4-t- |
| | | | | BuPh | BuPh |
| 122103 | t-Bu | t-Bu | t-Bu | 1,3- | 1,3- |
| | | | | di-t- | di-t- |
| | | | | BuPh | BuPh |
| 122104 | t-Bu | t-Bu | Ph | 1,3- | 1,3- |
| | | | | di-t- | di-t- |
| | | | | BuPh | BuPh |
| 122105 | t-Bu | t-Bu | CN | 1,3- | 1,3- |
| | | | | di-t- | di-t- |
| | | | | BuPh | BuPh |
| 122106 | t-Bu | t-Bu | DPA | 1,3- | 1,3- |
| | | | | di-t- | di-t- |
| | | | | BuPh | BuPh |
| 122107 | t-Bu | t-Bu | 2- | 1,3- | 1,3- |
| | | | DBF | di-t- | di-t- |
| | | | | BuPh | BuPh |
| 122108 | t-Bu | t-Bu | 2- | 1,3- | 1,3- |
| | | | DBT | di-t- | di-t- |
| | | | | BuPh | BuPh |
| 122109 | t-Bu | t-Bu | 4-t- | 1,3- | 1,3- |
| | | | BuPh | di-t- | di-t- |
| | | | | BuPh | BuPh |
| 122110 | t-Bu | t-Bu | 1,3- | 1,3- | 1,3- |
| | | | di-t- | di-t- | di-t- |
| | | | BuPh | BuPh | BuPh |
| 122111 | t-Bu | t-Bu | 9-Cbz | 1,3- | 1,3- |
| | | | | di-t- | di-t- |
| | | | | BuPh | BuPh |
| 122112 | Ph | Ph | t-Bu | H | H |
| 122113 | Ph | Ph | CN | H | H |
| 122114 | Ph | Ph | DPA | H | H |
| 122115 | Ph | Ph | 2- | H | H |
| | | | DBF | | |
| 122116 | Ph | Ph | 2- | H | H |
| | | | DBT | | |
| 122117 | Ph | Ph | 4-t- | H | H |
| | | | BuPh | | |
| 122118 | Ph | Ph | 1,3- | H | H |
| | | | di-t- | | |
| | | | BuPh | | |
| 122119 | Ph | Ph | 9-Cbz | H | H |
| 122120 | Ph | Ph | t-Bu | t-Bu | t-Bu |
| 122121 | Ph | Ph | Ph | t-Bu | t-Bu |
| 122122 | Ph | Ph | CN | t-Bu | t-Bu |
| 122123 | Ph | Ph | DPA | t-Bu | t-Bu |
| 122124 | Ph | Ph | 2- | t-Bu | t-Bu |
| | | | DBF | | |
| 122125 | Ph | Ph | 2- | t-Bu | t-Bu |
| | | | DBT | | |
| 122126 | Ph | Ph | 4-t- | t-Bu | t-Bu |
| | | | BuPh | | |
| 122127 | Ph | Ph | 1,3- | t-Bu | t-Bu |
| | | | di-t- | | |
| | | | BuPh | | |
| 122128 | Ph | Ph | 9-Cbz | t-Bu | t-Bu |
| 122129 | Ph | Ph | Ph | Ph | Ph |
| 122130 | Ph | Ph | CN | Ph | Ph |
| 122131 | Ph | Ph | DPA | Ph | Ph |
| 122132 | Ph | Ph | 2- | Ph | Ph |
| | | | DBF | | |
| 122133 | Ph | Ph | 2- | Ph | Ph |
| | | | DBT | | |
| 122134 | Ph | Ph | 4-t- | Ph | Ph |
| | | | BuPh | | |
| 122135 | Ph | Ph | 1,3- | Ph | Ph |
| | | | di-t- | | |
| | | | BuPh | | |
| 122136 | Ph | Ph | 9-Cbz | Ph | Ph |
| 122137 | Ph | Ph | t-Bu | CN | CN |
| 122138 | Ph | Ph | Ph | CN | CN |
| 122139 | Ph | Ph | CN | CN | CN |
| 122140 | Ph | Ph | DPA | CN | CN |
| 122141 | Ph | Ph | 2- | CN | CN |
| | | | DBF | | |
| 122142 | Ph | Ph | 2- | CN | CN |
| | | | DBT | | |
| 122143 | Ph | Ph | 4-t- | CN | CN |
| | | | BuPh | | |
| 122144 | Ph | Ph | 1,3- | CN | CN |
| | | | di-t- | | |
| | | | BuPh | | |
| 122145 | Ph | Ph | 9-Cbz | CN | CN |
| 122146 | Ph | Ph | t-Bu | 9-Cbz | 9-Cbz |
| 122147 | Ph | Ph | Ph | 9-Cbz | 9-Cbz |
| 122148 | Ph | Ph | CN | 9-Cbz | 9-Cbz |
| 122149 | Ph | Ph | DPA | 9-Cbz | 9-Cbz |
| 122150 | Ph | Ph | 2- | 9-Cbz | 9-Cbz |
| | | | DBF | | |
| 122151 | Ph | Ph | 2- | 9-Cbz | 9-Cbz |
| | | | DBT | | |
| 122152 | Ph | Ph | 4-t- | 9-Cbz | 9-Cbz |
| | | | BuPh | | |
| 122153 | Ph | Ph | 1,3- | 9-Cbz | 9-Cbz |
| | | | di-t- | | |
| | | | BuPh | | |
| 122154 | Ph | Ph | 9-Cbz | 9-Cbz | 9-Cbz |
| 122155 | Ph | Ph | t-Bu | 4-t- | 4-t- |
| | | | | BuPh | BuPh |
| 122156 | Ph | Ph | Ph | 4-t- | 4-t- |
| | | | | BuPh | BuPh |
| 122157 | Ph | Ph | CN | 4-t- | 4-t- |
| | | | | BuPh | BuPh |
| 122158 | Ph | Ph | DPA | 4-t- | 4-t- |
| | | | | BuPh | BuPh |
| 122159 | Ph | Ph | 2- | 4-t- | 4-t- |
| | | | DBF | BuPh | BuPh |
| 122160 | Ph | Ph | 2- | 4-t- | 4-t- |
| | | | DBT | BuPh | BuPh |
| 122161 | Ph | Ph | 4-t- | 4-t- | 4-t- |
| | | | BuPh | BuPh | BuPh |
| 122162 | Ph | Ph | 1,3- | 4-t- | 4-t- |
| | | | di-t- | BuPh | BuPh |
| | | | BuPh | | |
| 122163 | Ph | Ph | 9-Cbz | 4-t- | 4-t- |
| | | | | BuPh | BuPh |
| 122164 | Ph | Ph | t-Bu | 1,3- | 1,3- |
| | | | | di-t- | di-t- |
| | | | | BuPh | BuPh |
| 122165 | Ph | Ph | Ph | 1,3- | 1,3- |
| | | | | di-t- | di-t- |
| | | | | BuPh | BuPh |
| 122166 | Ph | Ph | CN | 1,3- | 1,3- |
| | | | | di-t- | di-t- |
| | | | | BuPh | BuPh |
| 122167 | Ph | Ph | DPA | 1,3- | 1,3- |
| | | | | di-t- | di-t- |
| | | | | BuPh | BuPh |
| 122168 | Ph | Ph | 2- | 1,3- | 1,3- |
| | | | DBF | di-t- | di-t- |
| | | | | BuPh | BuPh |
| 122169 | Ph | Ph | 2- | 1,3- | 1,3- |
| | | | DBT | di-t- | di-t- |
| | | | | BuPh | BuPh |
| 122170 | Ph | Ph | 4-t- | 1,3- | 1,3- |
| | | | BuPh | di-t- | di-t- |
| | | | | BuPh | BuPh |
| 122171 | Ph | Ph | 1,3- | 1,3- | 1,3- |
| | | | di-t- | di-t- | di-t- |
| | | | BuPh | BuPh | BuPh |
| 122172 | Ph | Ph | 9-Cbz | 1,3- | 1,3- |
| | | | | di-t- | di-t- |
| | | | | BuPh | BuPh |
| 122173 | 9-Cbz | 9-Cbz | t-Bu | H | H |
| 122174 | 9-Cbz | 9-Cbz | Ph | H | H |
| 122175 | 9-Cbz | 9-Cbz | CN | H | H |
| 122176 | 9-Cbz | 9-Cbz | DPA | H | H |
| 122177 | 9-Cbz | 9-Cbz | 2- | H | H |
| | | | DBF | | |
| 122178 | 9-Cbz | 9-Cbz | 2- | H | H |
| | | | DBT | | |
| 122179 | 9-Cbz | 9-Cbz | 4-t- | H | H |
| | | | BuPh | | |
| 122180 | 9-Cbz | 9-Cbz | 1,3- | H | H |
| | | | di-t- | | |
| | | | BuPh | | |
| 122181 | 9-Cbz | 9-Cbz | t-Bu | t-Bu | t-Bu |
| 122182 | 9-Cbz | 9-Cbz | Ph | t-Bu | t-Bu |
| 122183 | 9-Cbz | 9-Cbz | CN | t-Bu | t-Bu |
| 122184 | 9-Cbz | 9-Cbz | DPA | t-Bu | t-Bu |
| 122185 | 9-Cbz | 9-Cbz | 2- | t-Bu | t-Bu |
| | | | DBF | | |
| 122186 | 9-Cbz | 9-Cbz | 2- | t-Bu | t-Bu |
| | | | DBT | | |
| 122187 | 9-Cbz | 9-Cbz | 4-t- | t-Bu | t-Bu |
| | | | BuPh | | |
| 122188 | 9-Cbz | 9-Cbz | 1,3- | t-Bu | t-Bu |
| | | | di-t- | | |
| | | | BuPh | | |
| 122189 | 9-Cbz | 9-Cbz | 9-Cbz | t-Bu | t-Bu |
| 122190 | 9-Cbz | 9-Cbz | t-Bu | Ph | Ph |
| 122191 | 9-Cbz | 9-Cbz | Ph | Ph | Ph |
| 122192 | 9-Cbz | 9-Cbz | CN | Ph | Ph |
| 122193 | 9-Cbz | 9-Cbz | DPA | Ph | Ph |
| 122194 | 9-Cbz | 9-Cbz | 2- | Ph | Ph |
| | | | DBF | | |
| 122195 | 9-Cbz | 9-Cbz | 2- | Ph | Ph |
| | | | DBT | | |
| 122196 | 9-Cbz | 9-Cbz | 4-t- | Ph | Ph |
| | | | BuPh | | |
| 122197 | 9-Cbz | 9-Cbz | 1,3- | Ph | Ph |
| | | | di-t- | | |
| | | | BuPh | | |
| 122198 | 9-Cbz | 9-Cbz | 9-Cbz | Ph | Ph |
| 122199 | 9-Cbz | 9-Cbz | t-Bu | CN | CN |
| 122200 | 9-Cbz | 9-Cbz | Ph | CN | CN |
| 122201 | 9-Cbz | 9-Cbz | CN | CN | CN |
| 122202 | 9-Cbz | 9-Cbz | DPA | CN | CN |
| 122203 | 9-Cbz | 9-Cbz | 2- | CN | CN |
| | | | DBF | | |
| 122204 | 9-Cbz | 9-Cbz | 2- | CN | CN |
| | | | DBT | | |
| 122205 | 9-Cbz | 9-Cbz | 4-t- | CN | CN |
| | | | BuPh | | |
| 122206 | 9-Cbz | 9-Cbz | 1,3- | CN | CN |
| | | | di-t- | | |
| | | | BuPh | | |
| 122207 | 9-Cbz | 9-Cbz | 9-Cbz | CN | CN |
| 122208 | 9-Cbz | 9-Cbz | t-Bu | 9-Cbz | 9-Cbz |
| 122209 | 9-Cbz | 9-Cbz | Ph | 9-Cbz | 9-Cbz |
| 122210 | 9-Cbz | 9-Cbz | CN | 9-Cbz | 9-Cbz |
| 122211 | 9-Cbz | 9-Cbz | DPA | 9-Cbz | 9-Cbz |
| 122212 | 9-Cbz | 9-Cbz | 2- | 9-Cbz | 9-Cbz |
| | | | DBF | | |
| 122213 | 9-Cbz | 9-Cbz | 2- | 9-Cbz | 9-Cbz |
| | | | DBT | | |
| 122214 | 9-Cbz | 9-Cbz | 4-t- | 9-Cbz | 9-Cbz |
| | | | BuPh | | |
| 122215 | 9-Cbz | 9-Cbz | 1,3- | 9-Cbz | 9-Cbz |
| | | | di-t- | | |
| | | | BuPh | | |
| 122216 | 9-Cbz | 9-Cbz | 9-Cbz | 9-Cbz | 9-Cbz |
| 122217 | 9-Cbz | 9-Cbz | t-Bu | 4-t- | 4-t- |
| | | | | BuPh | BuPh |
| 122218 | 9-Cbz | 9-Cbz | Ph | 4-t- | 4-t- |
| | | | | BuPh | BuPh |
| 122219 | 9-Cbz | 9-Cbz | CN | 4-t- | 4-t- |
| | | | | BuPh | BuPh |
| 122220 | 9-Cbz | 9-Cbz | DPA | 4-t- | 4-t- |
| | | | | BuPh | BuPh |
| 122221 | 9-Cbz | 9-Cbz | 2- | 4-t- | 4-t- |
| | | | DBF | BuPh | BuPh |
| 122222 | 9-Cbz | 9-Cbz | 2- | 4-t- | 4-t- |
| | | | DBT | BuPh | BuPh |
| 122223 | 9-Cbz | 9-Cbz | 4-t- | 4-t- | 4-t- |
| | | | BuPh | BuPh | BuPh |
| 122224 | 9-Cbz | 9-Cbz | 1,3- | 4-t- | 4-t- |
| | | | di-t- | BuPh | BuPh |
| | | | BuPh | | |
| 122225 | 9-Cbz | 9-Cbz | 9-Cbz | 4-t- | 4-t- |
| | | | | BuPh | BuPh |
| 122226 | 9-Cbz | 9-Cbz | t-Bu | 1,3- | 1,3- |
| | | | | di-t- | di-t- |
| | | | | BuPh | BuPh |
| 122227 | 9-Cbz | 9-Cbz | Ph | 1,3- | 1,3- |
| | | | | di-t- | di-t- |
| | | | | BuPh | BuPh |
| 122228 | 9-Cbz | 9-Cbz | CN | 1,3- | 1,3- |
| | | | | di-t- | di-t- |
| | | | | BuPh | BuPh |
| 122229 | 9-Cbz | 9-Cbz | DPA | 1,3- | 1,3- |
| | | | | di-t- | di-t- |
| | | | | BuPh | BuPh |
| 122230 | 9-Cbz | 9-Cbz | 2- | 1,3- | 1,3- |
| | | | DBF | di-t- | di-t- |
| | | | | BuPh | BuPh |
| 122231 | 9-Cbz | 9-Cbz | 2- | 1,3- | 1,3- |
| | | | DBT | di-t- | di-t- |
| | | | | BuPh | BuPh |
| 122232 | 9-Cbz | 9-Cbz | 4-t- | 1,3- | 1,3- |
| | | | BuPh | di-t- | di-t- |
| | | | | BuPh | BuPh |
| 122233 | 9-Cbz | 9-Cbz | 1,3- | 1,3- | 1,3- |
| | | | di-t- | di-t- | di-t- |
| | | | BuPh | BuPh | BuPh |
| 122234 | 9-Cbz | 9-Cbz | 9-Cbz | 1,3- | 1,3- |
| | | | | di-t- | di-t- |
| | | | | BuPh | BuPh |
| 122235 | CN | CN | t-Bu | H | H |
| 122236 | CN | CN | Ph | H | H |
| 122237 | CN | CN | DPA | H | H |
| 122238 | CN | CN | 2- | H | H |
| | | | DBF | | |
| 122239 | CN | CN | 2- | H | H |
| | | | DBT | | |
| 122240 | CN | CN | 4-t- | H | H |
| | | | BuPh | | |
| 122241 | CN | CN | 1,3- | H | H |
| | | | di-t- | | |
| | | | BuPh | | |
| 122242 | CN | CN | 9-Cbz | H | H |
| 122243 | CN | CN | t-Bu | t-Bu | t-Bu |
| 122244 | CN | CN | Ph | t-Bu | t-Bu |
| 122245 | CN | CN | CN | t-Bu | t-Bu |
| 122246 | CN | CN | DPA | t-Bu | t-Bu |
| 122247 | CN | CN | 2- | t-Bu | t-Bu |
| | | | DBF | | |
| 122248 | CN | CN | 2- | t-Bu | t-Bu |
| | | | DBT | | |
| 122249 | CN | CN | 4-t- | t-Bu | t-Bu |
| | | | BuPh | | |
| 122250 | CN | CN | 1,3- | t-Bu | t-Bu |
| | | | di-t- | | |
| | | | BuPh | | |
| 122251 | CN | CN | 9-Cbz | t-Bu | t-Bu |
| 122252 | CN | CN | t-Bu | Ph | Ph |
| 122253 | CN | CN | Ph | Ph | Ph |
| 122254 | CN | CN | CN | Ph | Ph |
| 122255 | CN | CN | DPA | Ph | Ph |
| 122256 | CN | CN | 2- | Ph | Ph |
| | | | DBF | | |
| 122257 | CN | CN | 2- | Ph | Ph |
| | | | DBT | | |
| 122258 | CN | CN | 4-t- | Ph | Ph |
| | | | BuPh | | |
| 122259 | CN | CN | 1,3- | Ph | Ph |
| | | | di-t- | | |
| | | | BuPh | | |
| 122260 | CN | CN | 9-Cbz | Ph | Ph |
| 122261 | CN | CN | t-Bu | CN | CN |
| 122262 | CN | CN | Ph | CN | CN |
| 122263 | CN | CN | CN | CN | CN |
| 122264 | CN | CN | DPA | CN | CN |
| 122265 | CN | CN | 2- | CN | CN |
| | | | DBF | | |
| 122266 | CN | CN | 2- | CN | CN |
| | | | DBT | | |
| 122267 | CN | CN | 4-t- | CN | CN |
| | | | BuPh | | |
| 122268 | CN | CN | 1,3- | CN | CN |
| | | | di-t- | | |
| | | | BuPh | | |
| 122269 | CN | CN | 9-Cbz | CN | CN |
| 122270 | CN | CN | t-Bu | 9-Cbz | 9-Cbz |
| 122271 | CN | CN | Ph | 9-Cbz | 9-Cbz |
| 122272 | CN | CN | CN | 9-Cbz | 9-Cbz |
| 122273 | CN | CN | DPA | 9-Cbz | 9-Cbz |
| 122274 | CN | CN | 2- | 9-Cbz | 9-Cbz |
| | | | DBF | | |
| 122275 | CN | CN | 2- | 9-Cbz | 9-Cbz |
| | | | DBT | | |
| 122276 | CN | CN | 4-t- | 9-Cbz | 9-Cbz |
| | | | BuPh | | |
| 122277 | CN | CN | 1,3- | 9-Cbz | 9-Cbz |
| | | | di-t- | | |
| | | | BuPh | | |
| 122278 | CN | CN | 9-Cbz | 9-Cbz | 9-Cbz |
| 122279 | CN | CN | t-Bu | 4-t- | 4-t- |
| | | | | BuPh | BuPh |
| 122280 | CN | CN | Ph | 4-t- | 4-t- |
| | | | | BuPh | BuPh |
| 122281 | CN | CN | CN | 4-t- | 4-t- |
| | | | | BuPh | BuPh |
| 122282 | CN | CN | DPA | 4-t- | 4-t- |
| | | | | BuPh | BuPh |
| 122283 | CN | CN | 2- | 4-t- | 4-t- |
| | | | DBF | BuPh | BuPh |
| 122284 | CN | CN | 2- | 4-t- | 4-t- |
| | | | DBT | BuPh | BuPh |
| 122285 | CN | CN | 4-t- | 4-t- | 4-t- |
| | | | BuPh | BuPh | BuPh |
| 122286 | CN | CN | 1,3- | 4-t- | 4-t- |
| | | | di-t- | BuPh | BuPh |
| | | | BuPh | | |
| 122287 | CN | CN | 9-Cbz | 4-t- | 4-t- |
| | | | | BuPh | BuPh |
| 122288 | CN | CN | t-Bu | 1,3- | 1,3- |
| | | | | di-t- | di-t- |
| | | | | BuPh | BuPh |
| 122289 | CN | CN | Ph | 1,3- | 1,3- |
| | | | | di-t- | di-t- |
| | | | | BuPh | BuPh |
| 122290 | CN | CN | CN | 1,3- | 1,3- |
| | | | | di-t- | di-t- |
| | | | | BuPh | BuPh |
| 122291 | CN | CN | DPA | 1,3- | 1,3- |
| | | | | di-t- | di-t- |
| | | | | BuPh | BuPh |
| 122292 | CN | CN | 2- | 1,3- | 1,3- |
| | | | DBF | di-t- | di-t- |
| | | | | BuPh | BuPh |
| 122293 | CN | CN | 2- | 1,3- | 1,3- |
| | | | DBT | di-t- | di-t- |
| | | | | BuPh | BuPh |
| 122294 | CN | CN | 4-t- | 1,3- | 1,3- |
| | | | BuPh | di-t- | di-t- |
| | | | | BuPh | BuPh |
| 122295 | CN | CN | 1,3- | 1,3- | 1,3- |
| | | | di-t- | di-t- | di-t- |
| | | | BuPh | BuPh | BuPh |
| 122296 | CN | CN | 9-Cbz | 1,3- | 1,3- |
| | | | | di-t- | di-t- |
| | | | | BuPh | BuPh |
| 122297 | 4-t- | 4-t- | t-Bu | H | H |
| | BuPh | BuPh | | | |
| 122298 | 4-t- | 4-t- | Ph | H | H |
| | BuPh | BuPh | | | |
| 122299 | 4-t- | 4-t- | CN | H | H |
| | BuPh | BuPh | | | |
| 122300 | 4-t- | 4-t- | DPA | H | H |
| | BuPh | BuPh | | | |
| 122301 | 4-t- | 4-t- | 2- | H | H |
| | BuPh | BuPh | DBF | | |
| 122302 | 4-t- | 4-t- | 2- | H | H |
| | BuPh | BuPh | DBT | | |
| 122303 | 4-t- | 4-t- | 1,3- | H | H |
| | BuPh | BuPh | di-t- | | |
| | | | BuPh | | |
| 122304 | 4-t- | 4-t- | 9-Cbz | H | H |
| | BuPh | BuPh | | | |
| 122305 | 4-t- | 4-t- | t-Bu | t-Bu | t-Bu |
| | BuPh | BuPh | | | |
| 122306 | 4-t- | 4-t- | Ph | t-Bu | t-Bu |
| | BuPh | BuPh | | | |
| 122307 | 4-t- | 4-t- | CN | t-Bu | t-Bu |
| | BuPh | BuPh | | | |
| 122308 | 4-t- | 4-t- | DPA | t-Bu | t-Bu |
| | BuPh | BuPh | | | |
| 122309 | 4-t- | 4-t- | 2- | t-Bu | t-Bu |
| | BuPh | BuPh | DBF | | |
| 122310 | 4-t- | 4-t- | 2- | t-Bu | t-Bu |
| | BuPh | BuPh | DBT | | |
| 122311 | 4-t- | 4-t- | 4-t- | t-Bu | t-Bu |
| | BuPh | BuPh | BuPh | | |
| 122312 | 4-t- | 4-t- | 1,3- | t-Bu | t-Bu |
| | BuPh | BuPh | di-t- | | |
| | | | BuPh | | |
| 122313 | 4-t- | 4-t- | 9-Cbz | t-Bu | t-Bu |
| | BuPh | BuPh | | | |
| 122314 | 4-t- | 4-t- | t-Bu | Ph | Ph |
| | BuPh | BuPh | | | |
| 122315 | 4-t- | 4-t- | Ph | Ph | Ph |
| | BuPh | BuPh | | | |
| 122316 | 4-t- | 4-t- | CN | Ph | Ph |
| | BuPh | BuPh | | | |
| 122317 | 4-t- | 4-t- | DPA | Ph | Ph |
| | BuPh | BuPh | | | |
| 122318 | 4-t- | 4-t- | 2- | Ph | Ph |
| | BuPh | BuPh | DBF | | |
| 122319 | 4-t- | 4-t- | 2- | Ph | Ph |
| | BuPh | BuPh | DBT | | |
| 122320 | 4-t- | 4-t- | 4-t- | Ph | Ph |
| | BuPh | BuPh | BuPh | | |
| 122321 | 4-t- | 4-t- | 1,3- | Ph | Ph |
| | BuPh | BuPh | di-t- | | |
| | | | BuPh | | |
| 122322 | 4-t- | 4-t- | 9-Cbz | Ph | Ph |
| | BuPh | BuPh | | | |
| 122323 | 4-t- | 4-t- | t-Bu | CN | CN |
| | BuPh | BuPh | | | |
| 122324 | 4-t- | 4-t- | Ph | CN | CN |
| | BuPh | BuPh | | | |
| 122325 | 4-t- | 4-t- | CN | CN | CN |
| | BuPh | BuPh | | | |
| 122326 | 4-t- | 4-t- | DPA | CN | CN |
| | BuPh | BuPh | | | |
| 122327 | 4-t- | 4-t- | 2- | CN | CN |
| | BuPh | BuPh | DBF | | |
| 122328 | 4-t- | 4-t- | 2- | CN | CN |
| | BuPh | BuPh | DBT | | |
| 122329 | 4-t- | 4-t- | 4-t- | CN | CN |
| | BuPh | BuPh | BuPh | | |
| 122330 | 4-t- | 4-t- | 1,3- | CN | CN |
| | BuPh | BuPh | di-t- | | |
| | | | BuPh | | |
| 122331 | 4-t- | 4-t- | 9-Cbz | CN | CN |
| | BuPh | BuPh | | | |
| 122332 | 4-t- | 4-t- | t-Bu | 9-Cbz | 9-Cbz |
| | BuPh | BuPh | | | |
| 122333 | 4-t- | 4-t- | Ph | 9-Cbz | 9-Cbz |
| | BuPh | BuPh | | | |
| 122334 | 4-t- | 4-t- | CN | 9-Cbz | 9-Cbz |
| | BuPh | BuPh | | | |
| 122335 | 4-t- | 4-t- | DPA | 9-Cbz | 9-Cbz |
| | BuPh | BuPh | | | |
| 122336 | 4-t- | 4-t- | 2- | 9-Cbz | 9-Cbz |
| | BuPh | BuPh | DBF | | |
| 122337 | 4-t- | 4-t- | 2- | 9-Cbz | 9-Cbz |
| | BuPh | BuPh | DBT | | |
| 122338 | 4-t- | 4-t- | 4-t- | 9-Cbz | 9-Cbz |
| | BuPh | BuPh | BuPh | | |
| 122339 | 4-t- | 4-t- | 1,3- | 9-Cbz | 9-Cbz |
| | BuPh | BuPh | di-t- | | |
| | | | BuPh | | |
| 122340 | 4-t- | 4-t- | 9-Cbz | 9-Cbz | 9-Cbz |
| | BuPh | BuPh | | | |
| 122341 | 4-t- | 4-t- | t-Bu | 4-t- | 4-t- |
| | BuPh | BuPh | | BuPh | BuPh |
| 122342 | 4-t- | 4-t- | Ph | 4-t- | 4-t- |
| | BuPh | BuPh | | BuPh | BuPh |
| 122343 | 4-t- | 4-t- | CN | 4-t- | 4-t- |
| | BuPh | BuPh | | BuPh | BuPh |
| 122344 | 4-t- | 4-t- | DPA | 4-t- | 4-t- |
| | BuPh | BuPh | | BuPh | BuPh |
| 122345 | 4-t- | 4-t- | 2- | 4-t- | 4-t- |
| | BuPh | BuPh | DBF | BuPh | BuPh |
| 122346 | 4-t- | 4-t- | 2- | 4-t- | 4-t- |
| | BuPh | BuPh | DBT | BuPh | BuPh |
| 122347 | 4-t- | 4-t- | 4-t- | 4-t- | 4-t- |
| | BuPh | BuPh | BuPh | BuPh | BuPh |
| 122348 | 4-t- | 4-t- | 1,3- | 4-t- | 4-t- |
| | BuPh | BuPh | di-t- | BuPh | BuPh |
| | | | BuPh | | |
| 122349 | 4-t- | 4-t- | 9-Cbz | 4-t- | 4-t- |
| | BuPh | BuPh | | BuPh | BuPh |
| 122350 | 4-t- | 4-t- | t-Bu | 1,3- | 1,3- |
| | BuPh | BuPh | | di-t- | di-t- |
| | | | | BuPh | BuPh |
| 122351 | 4-t- | 4-t- | Ph | 1,3- | 1,3- |
| | BuPh | BuPh | | di-t- | di-t- |
| | | | | BuPh | BuPh |
| 122352 | 4-t- | 4-t- | CN | 1,3- | 1,3- |
| | BuPh | BuPh | | di-t- | di-t- |
| | | | | BuPh | BuPh |
| 122353 | 4-t- | 4-t- | DPA | 1,3- | 1,3- |
| | BuPh | BuPh | | di-t- | di-t- |
| | | | | BuPh | BuPh |
| 122354 | 4-t- | 4-t- | 2- | 1,3- | 1,3- |
| | BuPh | BuPh | DBF | di-t- | di-t- |
| | | | | BuPh | BuPh |
| 122355 | 4-t- | 4-t- | 2- | 1,3- | 1,3- |
| | BuPh | BuPh | DBT | di-t- | di-t- |
| | | | | BuPh | BuPh |
| 122356 | 4-t- | 4-t- | 4-t- | 1,3- | 1,3- |
| | BuPh | BuPh | BuPh | di-t- | di-t- |
| | | | | BuPh | BuPh |
| 122357 | 4-t- | 4-t- | 1,3- | 1,3- | 1,3- |
| | BuPh | BuPh | di-t- | di-t- | di-t- |
| | | | BuPh | BuPh | BuPh |
| 122358 | 4-t- | 4-t- | 9-Cbz | 1,3- | 1,3- |
| | BuPh | BuPh | | di-t- | di-t- |
| | | | | BuPh | BuPh |
| 122359 | 1,3- | 1,3- | t-Bu | H | H |
| | di-t- | di-t- | | | |
| | BuPh | BuPh | | | |
| 122360 | 1,3- | 1,3- | Ph | H | H |
| | di-t- | di-t- | | | |
| | BuPh | BuPh | | | |
| 122361 | 1,3- | 1,3- | CN | H | H |
| | di-t- | di-t- | | | |
| | BuPh | BuPh | | | |
| 122362 | 1,3- | 1,3- | DPA | H | H |
| | di-t- | di-t- | | | |
| | BuPh | BuPh | | | |
| 122363 | 1,3- | 1,3- | 2- | H | H |
| | di-t- | di-t- | DBF | | |
| | BuPh | BuPh | | | |
| 122364 | 1,3- | 1,3- | 2- | H | H |
| | di-t- | di-t- | DBT | | |
| | BuPh | BuPh | | | |
| 122365 | 1,3- | 1,3- | 4-t- | H | H |
| | di-t- | di-t- | BuPh | | |
| | BuPh | BuPh | | | |
| 122366 | 1,3- | 1,3- | 9-Cbz | H | H |
| | di-t- | di-t- | | | |
| | BuPh | BuPh | | | |
| 122367 | 1,3- | 1,3- | t-Bu | t-Bu | t-Bu |
| | di-t- | di-t- | | | |
| | BuPh | BuPh | | | |
| 122368 | 1,3- | 1,3- | Ph | t-Bu | t-Bu |
| | di-t- | di-t- | | | |
| | BuPh | BuPh | | | |
| 122369 | 1,3- | 1,3- | CN | t-Bu | t-Bu |
| | di-t- | di-t- | | | |
| | BuPh | BuPh | | | |
| 122370 | 1,3- | 1,3- | DPA | t-Bu | t-Bu |
| | di-t- | di-t- | | | |
| | BuPh | BuPh | | | |
| 122371 | 1,3- | 1,3- | 2- | t-Bu | t-Bu |
| | di-t- | di-t- | DBF | | |
| | BuPh | BuPh | | | |
| 122372 | 1,3- | 1,3- | 2- | t-Bu | t-Bu |
| | di-t- | di-t- | DBT | | |
| | BuPh | BuPh | | | |
| 122373 | 1,3- | 1,3- | 4-t- | t-Bu | t-Bu |
| | di-t- | di-t- | BuPh | | |
| | BuPh | BuPh | | | |
| 122374 | 1,3- | 1,3- | 1,3- | t-Bu | t-Bu |
| | di-t- | di-t- | di-t- | | |
| | BuPh | BuPh | BuPh | | |
| 122375 | 1,3- | 1,3- | 9-Cbz | t-Bu | t-Bu |
| | di-t- | di-t- | | | |
| | BuPh | BuPh | | | |
| 122376 | 1,3- | 1,3- | t-Bu | Ph | Ph |
| | di-t- | di-t- | | | |
| | BuPh | BuPh | | | |
| 122377 | 1,3- | 1,3- | Ph | Ph | Ph |
| | di-t- | di-t- | | | |
| | BuPh | BuPh | | | |
| 122378 | 1,3- | 1,3- | CN | Ph | Ph |
| | di-t- | di-t- | | | |
| | BuPh | BuPh | | | |
| 122379 | 1,3- | 1,3- | DPA | Ph | Ph |
| | di-t- | di-t- | | | |
| | BuPh | BuPh | | | |
| 122380 | 1,3- | 1,3- | 2- | Ph | Ph |
| | di-t- | di-t- | DBF | | |
| | BuPh | BuPh | | | |
| 122381 | 1,3- | 1,3- | 2- | Ph | Ph |
| | di-t- | di-t- | DBT | | |
| | BuPh | BuPh | | | |
| 122382 | 1,3- | 1,3- | 4-t- | Ph | Ph |
| | di-t- | di-t- | BuPh | | |
| | BuPh | BuPh | | | |
| 122383 | 1,3- | 1,3- | 1,3- | Ph | Ph |
| | di-t- | di-t- | di-t- | | |
| | BuPh | BuPh | BuPh | | |
| 122384 | 1,3- | 1,3- | 9-Cbz | Ph | Ph |
| | di-t- | di-t- | | | |
| | BuPh | BuPh | | | |
| 122385 | 1,3- | 1,3- | t-Bu | CN | CN |
| | di-t- | di-t- | | | |
| | BuPh | BuPh | | | |
| 122386 | 1,3- | 1,3- | Ph | CN | CN |
| | di-t- | di-t- | | | |
| | BuPh | BuPh | | | |
| 122387 | 1,3- | 1,3- | CN | CN | CN |
| | di-t- | di-t- | | | |
| | BuPh | BuPh | | | |
| 122388 | 1,3- | 1,3- | DPA | CN | CN |
| | di-t- | di-t- | | | |
| | BuPh | BuPh | | | |
| 122389 | 1,3- | 1,3- | 2- | CN | CN |
| | di-t- | di-t- | DBF | | |
| | BuPh | BuPh | | | |
| 122390 | 1,3- | 1,3- | 2- | CN | CN |
| | di-t- | di-t- | DBT | | |
| | BuPh | BuPh | | | |
| 122391 | 1,3- | 1,3- | 4-t- | CN | CN |
| | di-t- | di-t- | BuPh | | |
| | BuPh | BuPh | | | |
| 122392 | 1,3- | 1,3- | 1,3- | CN | CN |
| | di-t- | di-t- | di-t- | | |
| | BuPh | BuPh | BuPh | | |
| 122393 | 1,3- | 1,3- | 9-Cbz | CN | CN |
| | di-t- | di-t- | | | |
| | BuPh | BuPh | | | |
| 122394 | 1,3- | 1,3- | t-Bu | 9-Cbz | 9-Cbz |
| | di-t- | di-t- | | | |
| | BuPh | BuPh | | | |
| 122395 | 1,3- | 1,3- | Ph | 9-Cbz | 9-Cbz |
| | di-t- | di-t- | | | |
| | BuPh | BuPh | | | |
| 122396 | 1,3- | 1,3- | CN | 9-Cbz | 9-Cbz |
| | di-t- | di-t- | | | |
| | BuPh | BuPh | | | |
| 122397 | 1,3- | 1,3- | DPA | 9-Cbz | 9-Cbz |
| | di-t- | di-t- | | | |
| | BuPh | BuPh | | | |
| 122398 | 1,3- | 1,3- | 2- | 9-Cbz | 9-Cbz |
| | di-t- | di-t- | DBF | | |
| | BuPh | BuPh | | | |
| 122399 | 1,3- | 1,3- | 2- | 9-Cbz | 9-Cbz |
| | di-t- | di-t- | DBT | | |
| | BuPh | BuPh | | | |
| 122400 | 1,3- | 1,3- | 4-t- | 9-Cbz | 9-Cbz |
| | di-t- | di-t- | BuPh | | |
| | BuPh | BuPh | | | |
| 122401 | 1,3- | 1,3- | 1,3- | 9-Cbz | 9-Cbz |
| | di-t- | di-t- | di-t- | | |
| | BuPh | BuPh | BuPh | | |
| 122402 | 1,3- | 1,3- | 9-Cbz | 9-Cbz | 9-Cbz |
| | di-t- | di-t- | | | |
| | BuPh | BuPh | | | |
| 122403 | 1,3- | 1,3- | t-Bu | 4-t- | 4-t- |
| | di-t- | di-t- | | BuPh | BuPh |
| | BuPh | BuPh | | | |
| 122404 | 1,3- | 1,3- | Ph | 4-t- | 4-t- |
| | di-t- | di-t- | | BuPh | BuPh |
| | BuPh | BuPh | | | |
| 122405 | 1,3- | 1,3- | CN | 4-t- | 4-t- |
| | di-t- | di-t- | | BuPh | BuPh |
| | BuPh | BuPh | | | |
| 122406 | 1,3- | 1,3- | DPA | 4-t- | 4-t- |
| | di-t- | di-t- | | BuPh | BuPh |
| | BuPh | BuPh | | | |
| 122407 | 1,3- | 1,3- | 2- | 4-t- | 4-t- |
| | di-t- | di-t- | DBF | BuPh | BuPh |
| | BuPh | BuPh | | | |
| 122408 | 1,3- | 1,3- | 2- | 4-t- | 4-t- |
| | di-t- | di-t- | DBT | BuPh | BuPh |
| | BuPh | BuPh | | | |
| 122409 | 1,3- | 1,3- | 4-t- | 4-t- | 4-t- |
| | di-t- | di-t- | BuPh | BuPh | BuPh |
| | BuPh | BuPh | | | |
| 122410 | 1,3- | 1,3- | 1,3- | 4-t- | 4-t- |
| | di-t- | di-t- | di-t- | BuPh | BuPh |
| | BuPh | BuPh | BuPh | | |
| 122411 | 1,3- | 1,3- | 9-Cbz | 4-t- | 4-t- |
| | di-t- | di-t- | | BuPh | BuPh |
| | BuPh | BuPh | | | |
| 122412 | 1,3- | 1,3- | t-Bu | 1,3- | 1,3- |
| | di-t- | di-t- | | di-t- | di-t- |
| | BuPh | BuPh | | BuPh | BuPh |
| 122413 | 1,3- | 1,3- | Ph | 1,3- | 1,3- |
| | di-t- | di-t- | | di-t- | di-t- |
| | BuPh | BuPh | | BuPh | BuPh |
| 122414 | 1,3- | 1,3- | CN | 1,3- | 1,3- |
| | di-t- | di-t- | | di-t- | di-t- |
| | BuPh | BuPh | | BuPh | BuPh |
| 122415 | 1,3- | 1,3- | DPA | 1,3- | 1,3- |
| | di-t- | di-t- | | di-t- | di-t- |
| | BuPh | BuPh | | BuPh | BuPh |
| 122416 | 1,3- | 1,3- | 2- | 1,3- | 1,3- |
| | di-t- | di-t- | DBF | di-t- | di-t- |
| | BuPh | BuPh | | BuPh | BuPh |
| 122417 | 1,3- | 1,3- | 2- | 1,3- | 1,3- |
| | di-t- | di-t- | DBT | di-t- | di-t- |
| | BuPh | BuPh | | BuPh | BuPh |
| 122418 | 1,3- | 1,3- | 4-t- | 1,3- | 1,3- |
| | di-t- | di-t- | BuPh | di-t- | di-t- |
| | BuPh | BuPh | | BuPh | BuPh |
| 122419 | 1,3- | 1,3- | 1,3- | 1,3- | 1,3- |
| | di-t- | di-t- | di-t- | di-t- | di-t- |
| | BuPh | BuPh | BuPh | BuPh | BuPh |
| 122420 | 1,3- | 1,3- | 9-Cbz | 1,3- | 1,3- |
| | di-t- | di-t- | | di-t- | di-t- |
| | BuPh | BuPh | | BuPh | BuPh |

### Group HC5

The heterocyclic compound may be represented by Formula 12-4, and substituents may be defined as shown in Table 5.

TABLE 5

| Compound | | | | | |
|---|---|---|---|---|---|
| No. | R_{11b} | R_{12c} | R_{14b} | R_{18c} | R_{19b} |
| 124001 | t-Bu | t-Bu | t-Bu | t-Bu | t-Bu |
| 124002 | Ph | Ph | t-Bu | Ph | Ph |
| 124003 | t-Bu | t-Bu | H | H | H |
| 124004 | H | H | t-Bu | H | H |
| 124005 | H | H | H | t-Bu | t-Bu |
| 124006 | t-Bu | t-Bu | t-Bu | H | H |
| 124007 | H | H | t-Bu | t-Bu | t-Bu |
| 124008 | t-Bu | t-Bu | H | t-Bu | t-Bu |
| 124009 | Ph | Ph | H | H | H |
| 124010 | H | H | Ph | H | H |
| 124011 | H | H | H | Ph | Ph |
| 124012 | Ph | Ph | Ph | H | H |
| 124013 | H | H | Ph | Ph | Ph |
| 124014 | Ph | Ph | H | Ph | Ph |
| 124015 | CN | CN | H | H | H |
| 124016 | H | H | CN | H | H |
| 124017 | H | H | H | CN | CN |
| 124018 | CN | CN | CN | H | H |
| 124019 | H | H | CN | CN | CN |
| 124020 | CN | CN | H | CN | CN |
| 124021 | 9-Cbz | 9-Cbz | H | H | H |
| 124022 | H | H | 9-Cbz | H | H |
| 124023 | H | H | H | 9-Cbz | 9-Cbz |
| 124024 | 9-Cbz | 9-Cbz | 9-Cbz | H | H |
| 124025 | H | H | 9-Cbz | 9-Cbz | 9-Cbz |
| 124026 | 9-Cbz | 9-Cbz | H | 9-Cbz | 9-Cbz |
| 124027 | Me | Me | H | H | H |
| 124028 | H | H | Me | H | H |
| 124029 | H | H | H | Me | Me |
| 124030 | Me | Me | Me | H | H |
| 124031 | H | H | Me | Me | Me |
| 124032 | Me | Me | H | Me | Me |
| 124033 | CD₃ | CD3 | H | H | H |
| 124034 | H | H | CD3 | H | H |
| 124035 | H | H | H | CD3 | CD3 |
| 124036 | CD3 | CD3 | CD3 | H | H |
| 124037 | H | H | CD3 | CD3 | CD3 |
| 124038 | CD3 | CD3 | H | CD3 | CD3 |
| 124039 | 4-t- | 4-t- | H | H | H |
| | BuPh | BuPh | | | |
| 124040 | H | H | 4-t- | H | H |
| | | | BuPh | | |
| 124041 | H | H | H | 4-t- | 4-t- |
| | | | | BuPh | BuPh |
| 124042 | 4-t- | 4-t- | 4-t- | H | H |
| | BuPh | BuPh | BuPh | | |
| 124043 | H | H | 4-t- | 4-t- | 4-t- |
| | | | BuPh | BuPh | BuPh |
| 124044 | 4-t- | 4-t- | H | 4-t- | 4-t- |
| | BuPh | BuPh | | BuPh | BuPh |
| 124045 | 1,3- | 1,3- | H | H | H |
| | di-t- | di-t- | | | |
| | BuPh | BuPh | | | |
| 124046 | H | H | 1,3- | H | H |
| | | | di-t- | | |
| | | | BuPh | | |
| 124047 | H | H | H | 1,3- | 1,3- |
| | | | | di-t- | di-t- |
| | | | | BuPh | BuPh |
| 124048 | 1,3- | 1,3- | 1,3- | H | H |
| | di-t- | di-t- | di-t- | | |
| | BuPh | BuPh | BuPh | | |
| 124049 | H | H | 1,3- | 1,3- | 1,3- |
| | | | di-t- | di-t- | di-t- |
| | | | BuPh | BuPh | BuPh |
| 124050 | 1,3- | 1,3- | H | 1,3- | 1,3- |
| | di-t- | di-t- | | di-t- | di-t- |
| | BuPh | BuPh | | BuPh | BuPh |
| 124051 | t-Bu | t-Bu | Ph | H | H |
| 124052 | t-Bu | t-Bu | CN | H | H |
| 124053 | t-Bu | t-Bu | DPA | H | H |
| 124054 | t-Bu | t-Bu | 2- | H | H |
| | | | DBF | | |
| 124055 | t-Bu | t-Bu | 2- | H | H |
| | | | DBT | | |
| 124056 | t-Bu | t-Bu | 4-t- | H | H |
| | | | BuPh | | |
| 124057 | t-Bu | t-Bu | 1,3- | H | H |
| | | | di-t- | | |
| | | | BuPh | | |
| 124058 | t-Bu | t-Bu | 9-Cbz | H | H |
| 124059 | t-Bu | t-Bu | Ph | t-Bu | t-Bu |
| 124060 | t-Bu | t-Bu | CN | t-Bu | t-Bu |
| 124061 | t-Bu | t-Bu | DPA | t-Bu | t-Bu |
| 124062 | t-Bu | t-Bu | 2- | t-Bu | t-Bu |
| | | | DBF | | |
| 124063 | t-Bu | t-Bu | 2- | t-Bu | t-Bu |
| | | | DBT | | |
| 124064 | t-Bu | t-Bu | 4-t- | t-Bu | t-Bu |
| | | | BuPh | | |
| 124065 | t-Bu | t-Bu | 1,3- | t-Bu | t-Bu |
| | | | di-t- | | |
| | | | BuPh | | |
| 124066 | t-Bu | t-Bu | 9-Cbz | t-Bu | t-Bu |
| 124067 | t-Bu | t-Bu | t-Bu | Ph | Ph |
| 124068 | t-Bu | t-Bu | Ph | Ph | Ph |
| 124069 | t-Bu | t-Bu | CN | Ph | Ph |
| 124070 | t-Bu | t-Bu | DPA | Ph | Ph |
| 124071 | t-Bu | t-Bu | 2- | Ph | Ph |
| | | | DBF | | |
| 124072 | t-Bu | t-Bu | 2- | Ph | Ph |
| | | | DBT | | |
| 124073 | t-Bu | t-Bu | 4-t- | Ph | Ph |
| | | | BuPh | | |
| 124074 | t-Bu | t-Bu | 1,3- | Ph | Ph |
| | | | di-t- | | |
| | | | BuPh | | |
| 124075 | t-Bu | t-Bu | 9-Cbz | Ph | Ph |
| 124076 | t-Bu | t-Bu | t-Bu | CN | CN |
| 124077 | t-Bu | t-Bu | Ph | CN | CN |
| 124078 | t-Bu | t-Bu | CN | CN | CN |
| 124079 | t-Bu | t-Bu | DPA | CN | CN |
| 124080 | t-Bu | t-Bu | 2- | CN | CN |
| | | | DBF | | |
| 124081 | t-Bu | t-Bu | 2- | CN | CN |
| | | | DBT | | |
| 124082 | t-Bu | t-Bu | 4-t- | CN | CN |
| | | | BuPh | | |
| 124083 | t-Bu | t-Bu | 1,3- | CN | CN |
| | | | di-t- | | |
| | | | BuPh | | |
| 124084 | t-Bu | t-Bu | 9-Cbz | CN | CN |
| 124085 | t-Bu | t-Bu | t-Bu | 9-Cbz | 9-Cbz |
| 124086 | t-Bu | t-Bu | Ph | 9-Cbz | 9-Cbz |
| 124087 | t-Bu | t-Bu | CN | 9-Cbz | 9-Cbz |
| 124088 | t-Bu | t-Bu | DPA | 9-Cbz | 9-Cbz |
| 124089 | t-Bu | t-Bu | 2- | 9-Cbz | 9-Cbz |
| | | | DBF | | |
| 124090 | t-Bu | t-Bu | 2- | 9-Cbz | 9-Cbz |
| | | | DBT | | |
| 124091 | t-Bu | t-Bu | 4-t- | 9-Cbz | 9-Cbz |
| | | | BuPh | | |
| 124092 | t-Bu | t-Bu | 1,3- | 9-Cbz | 9-Cbz |
| | | | di-t- | | |
| | | | BuPh | | |
| 124093 | t-Bu | t-Bu | 9-Cbz | 9-Cbz | 9-Cbz |
| 124094 | t-Bu | t-Bu | t-Bu | 4-t- | 4-t- |
| | | | | BuPh | BuPh |
| 124095 | t-Bu | t-Bu | Ph | 4-t- | 4-t- |
| | | | | BuPh | BuPh |
| 124096 | t-Bu | t-Bu | CN | 4-t- | 4-t- |
| | | | | BuPh | BuPh |
| 124097 | t-Bu | t-Bu | DPA | 4-t- | 4-t- |
| | | | | BuPh | BuPh |
| 124098 | t-Bu | t-Bu | 2- | 4-t- | 4-t- |
| | | | DBF | BuPh | BuPh |
| 124099 | t-Bu | t-Bu | 2- | 4-t- | 4-t- |
| | | | DBT | BuPh | BuPh |
| 124100 | t-Bu | t-Bu | 4-t- | 4-t- | 4-t- |
| | | | BuPh | BuPh | BuPh |
| 124101 | t-Bu | t-Bu | 1,3- | 4-t- | 4-t- |
| | | | di-t- | BuPh | BuPh |
| | | | BuPh | | |
| 124102 | t-Bu | t-Bu | 9-Cbz | 4-t- | 4-t- |
| | | | | BuPh | BuPh |
| 124103 | t-Bu | t-Bu | t-Bu | 1,3- | 1,3- |
| | | | | di-t- | di-t- |
| | | | | BuPh | BuPh |
| 124104 | t-Bu | t-Bu | Ph | 1,3- | 1,3- |
| | | | | di-t- | di-t- |
| | | | | BuPh | BuPh |
| 124105 | t-Bu | t-Bu | CN | 1,3- | 1,3- |
| | | | | di-t- | di-t- |
| | | | | BuPh | BuPh |
| 124106 | t-Bu | t-Bu | DPA | 1,3- | 1,3- |
| | | | | di-t- | di-t- |
| | | | | BuPh | BuPh |
| 124107 | t-Bu | t-Bu | 2- | 1,3- | 1,3- |
| | | | DBF | di-t- | di-t- |
| | | | | BuPh | BuPh |
| 124108 | t-Bu | t-Bu | 2- | 1,3- | 1,3- |
| | | | DBT | di-t- | di-t- |
| | | | | BuPh | BuPh |
| 124109 | t-Bu | t-Bu | 4-t- | 1,3- | 1,3- |
| | | | BuPh | di-t- | di-t- |
| | | | | BuPh | BuPh |
| 124110 | t-Bu | t-Bu | 1,3- | 1,3- | 1,3- |
| | | | di-t- | di-t- | di-t- |
| | | | BuPh | BuPh | BuPh |
| 124111 | t-Bu | t-Bu | 9-Cbz | 1,3- | 1,3- |
| | | | | di-t- | di-t- |
| | | | | BuPh | BuPh |
| 124112 | Ph | Ph | t-Bu | H | H |
| 124113 | Ph | Ph | CN | H | H |
| 124114 | Ph | Ph | DPA | H | H |
| 124115 | Ph | Ph | 2- | H | H |
| | | | DBF | | |
| 124116 | Ph | Ph | 2- | H | H |
| | | | DBT | | |
| 124117 | Ph | Ph | 4-t- | H | H |
| | | | BuPh | | |
| 124118 | Ph | Ph | 1,3- | H | H |
| | | | di-t- | | |
| | | | BuPh | | |
| 124119 | Ph | Ph | 9-Cbz | H | H |
| 124120 | Ph | Ph | t-Bu | t-Bu | t-Bu |
| 124121 | Ph | Ph | Ph | t-Bu | t-Bu |
| 124122 | Ph | Ph | CN | t-Bu | t-Bu |
| 124123 | Ph | Ph | DPA | t-Bu | t-Bu |
| 124124 | Ph | Ph | 2- | t-Bu | t-Bu |
| | | | DBF | | |
| 124125 | Ph | Ph | 2- | t-Bu | t-Bu |
| | | | DBT | | |
| 124126 | Ph | Ph | 4-t- | t-Bu | t-Bu |
| | | | BuPh | | |
| 124127 | Ph | Ph | 1,3- | t-Bu | t-Bu |
| | | | di-t- | | |
| | | | BuPh | | |
| 124128 | Ph | Ph | 9-Cbz | t-Bu | t-Bu |
| 124129 | Ph | Ph | Ph | Ph | Ph |
| 124130 | Ph | Ph | CN | Ph | Ph |
| 124131 | Ph | Ph | DPA | Ph | Ph |
| 124132 | Ph | Ph | 2- | Ph | Ph |
| | | | DBF | | |
| 124133 | Ph | Ph | 2- | Ph | Ph |
| | | | DBT | | |
| 124134 | Ph | Ph | 4-t- | Ph | Ph |
| | | | BuPh | | |
| 124135 | Ph | Ph | 1,3- | Ph | Ph |
| | | | di-t- | | |
| | | | BuPh | | |
| 124136 | Ph | Ph | 9-Cbz | Ph | Ph |
| 124137 | Ph | Ph | t-Bu | CN | CN |
| 124138 | Ph | Ph | Ph | CN | CN |
| 124139 | Ph | Ph | CN | CN | CN |
| 124140 | Ph | Ph | DPA | CN | CN |
| 124141 | Ph | Ph | 2- | CN | CN |
| | | | DBF | | |
| 124142 | Ph | Ph | 2- | CN | CN |
| | | | DBT | | |
| 124143 | Ph | Ph | 4-t- | CN | CN |
| | | | BuPh | | |
| 124144 | Ph | Ph | 1,3- | CN | CN |
| | | | di-t- | | |
| | | | BuPh | | |
| 124145 | Ph | Ph | 9-Cbz | CN | CN |
| 124146 | Ph | Ph | t-Bu | 9-Cbz | 9-Cbz |
| 124147 | Ph | Ph | Ph | 9-Cbz | 9-Cbz |
| 124148 | Ph | Ph | CN | 9-Cbz | 9-Cbz |
| 124149 | Ph | Ph | DPA | 9-Cbz | 9-Cbz |
| 124150 | Ph | Ph | 2- | 9-Cbz | 9-Cbz |
| | | | DBF | | |
| 124151 | Ph | Ph | 2- | 9-Cbz | 9-Cbz |
| | | | DBT | | |
| 124152 | Ph | Ph | 4-t- | 9-Cbz | 9-Cbz |
| | | | BuPh | | |
| 124153 | Ph | Ph | 1,3- | 9-Cbz | 9-Cbz |
| | | | di-t- | | |
| | | | BuPh | | |
| 124154 | Ph | Ph | 9-Cbz | 9-Cbz | 9-Cbz |
| 124155 | Ph | Ph | t-Bu | 4-t- | 4-t- |
| | | | | BuPh | BuPh |
| 124156 | Ph | Ph | Ph | 4-t- | 4-t- |
| | | | | BuPh | BuPh |
| 124157 | Ph | Ph | CN | 4-t- | 4-t- |
| | | | | BuPh | BuPh |
| 124158 | Ph | Ph | DPA | 4-t- | 4-t- |
| | | | | BuPh | BuPh |
| 124159 | Ph | Ph | 2- | 4-t- | 4-t- |
| | | | DBF | BuPh | BuPh |
| 124160 | Ph | Ph | 2- | 4-t- | 4-t- |
| | | | DBT | BuPh | BuPh |
| 124161 | Ph | Ph | 4-t- | 4-t- | 4-t- |
| | | | BuPh | BuPh | BuPh |
| 124162 | Ph | Ph | 1,3- | 4-t- | 4-t- |
| | | | di-t- | BuPh | BuPh |
| | | | BuPh | | |
| 124163 | Ph | Ph | 9-Cbz | 4-t- | 4-t- |
| | | | | BuPh | BuPh |
| 124164 | Ph | Ph | t-Bu | 1,3- | 1,3- |
| | | | | di-t- | di-t- |
| | | | | BuPh | BuPh |
| 124165 | Ph | Ph | Ph | 1,3- | 1,3- |
| | | | | di-t- | di-t- |
| | | | | BuPh | BuPh |
| 124166 | Ph | Ph | CN | 1,3- | 1,3- |
| | | | | di-t- | di-t- |
| | | | | BuPh | BuPh |
| 124167 | Ph | Ph | DPA | 1,3- | 1,3- |
| | | | | di-t- | di-t- |
| | | | | BuPh | BuPh |
| 124168 | Ph | Ph | 2- | 1,3- | 1,3- |
| | | | DBF | di-t- | di-t- |
| | | | | BuPh | BuPh |
| 124169 | Ph | Ph | 2- | 1,3- | 1,3- |
| | | | DBT | di-t- | di-t- |
| | | | | BuPh | BuPh |
| 124170 | Ph | Ph | 4-t- | 1,3- | 1,3- |
| | | | BuPh | di-t- | di-t- |
| | | | | BuPh | BuPh |
| 124171 | Ph | Ph | 1,3- | 1,3- | 1,3- |
| | | | di-t- | di-t- | di-t- |
| | | | BuPh | BuPh | BuPh |
| 124172 | Ph | Ph | 9-Cbz | 1,3- | 1,3- |
| | | | | di-t- | di-t- |
| | | | | BuPh | BuPh |
| 124173 | 9-Cbz | 9-Cbz | t-Bu | H | H |
| 124174 | 9-Cbz | 9-Cbz | Ph | H | H |
| 124175 | 9-Cbz | 9-Cbz | CN | H | H |
| 124176 | 9-Cbz | 9-Cbz | DPA | H | H |
| 124177 | 9-Cbz | 9-Cbz | 2- | H | H |
| | | | DBF | | |
| 124178 | 9-Cbz | 9-Cbz | 2- | H | H |
| | | | DBT | | |
| 124179 | 9-Cbz | 9-Cbz | 4-t- | H | H |
| | | | BuPh | | |
| 124180 | 9-Cbz | 9-Cbz | 1,3- | H | H |
| | | | di-t- | | |
| | | | BuPh | | |
| 124181 | 9-Cbz | 9-Cbz | t-Bu | t-Bu | t-Bu |
| 124182 | 9-Cbz | 9-Cbz | Ph | t-Bu | t-Bu |
| 124183 | 9-Cbz | 9-Cbz | CN | t-Bu | t-Bu |
| 124184 | 9-Cbz | 9-Cbz | DPA | t-Bu | t-Bu |
| 124185 | 9-Cbz | 9-Cbz | 2- | t-Bu | t-Bu |
| | | | DBF | | |
| 124186 | 9-Cbz | 9-Cbz | 2- | t-Bu | t-Bu |
| | | | DBT | | |
| 124187 | 9-Cbz | 9-Cbz | 4-t- | t-Bu | t-Bu |
| | | | BuPh | | |
| 124188 | 9-Cbz | 9-Cbz | 1,3- | t-Bu | t-Bu |
| | | | di-t- | | |
| | | | BuPh | | |
| 124189 | 9-Cbz | 9-Cbz | 9-Cbz | t-Bu | t-Bu |
| 124190 | 9-Cbz | 9-Cbz | t-Bu | Ph | Ph |
| 124191 | 9-Cbz | 9-Cbz | Ph | Ph | Ph |
| 124192 | 9-Cbz | 9-Cbz | CN | Ph | Ph |
| 124193 | 9-Cbz | 9-Cbz | DPA | Ph | Ph |
| 124194 | 9-Cbz | 9-Cbz | 2- | Ph | Ph |
| | | | DBF | | |
| 124195 | 9-Cbz | 9-Cbz | 2- | Ph | Ph |
| | | | DBT | | |
| 124196 | 9-Cbz | 9-Cbz | 4-t- | Ph | Ph |
| | | | BuPh | | |
| 124197 | 9-Cbz | 9-Cbz | 1,3- | Ph | Ph |
| | | | di-t- | | |
| | | | BuPh | | |
| 124198 | 9-Cbz | 9-Cbz | 9-Cbz | Ph | Ph |
| 124199 | 9-Cbz | 9-Cbz | t-Bu | CN | CN |
| 124200 | 9-Cbz | 9-Cbz | Ph | CN | CN |
| 124201 | 9-Cbz | 9-Cbz | CN | CN | CN |
| 124202 | 9-Cbz | 9-Cbz | DPA | CN | CN |
| 124203 | 9-Cbz | 9-Cbz | 2- | CN | CN |
| | | | DBF | | |
| 124204 | 9-Cbz | 9-Cbz | 2- | CN | CN |
| | | | DBT | | |
| 124205 | 9-Cbz | 9-Cbz | 4-t- | CN | CN |
| | | | BuPh | | |
| 124206 | 9-Cbz | 9-Cbz | 1,3- | CN | CN |
| | | | di-t- | | |
| | | | BuPh | | |
| 124207 | 9-Cbz | 9-Cbz | 9-Cbz | CN | CN |
| 124208 | 9-Cbz | 9-Cbz | t-Bu | 9-Cbz | 9-Cbz |
| 124209 | 9-Cbz | 9-Cbz | Ph | 9-Cbz | 9-Cbz |
| 124210 | 9-Cbz | 9-Cbz | CN | 9-Cbz | 9-Cbz |
| 124211 | 9-Cbz | 9-Cbz | DPA | 9-Cbz | 9-Cbz |
| 124212 | 9-Cbz | 9-Cbz | 2- | 9-Cbz | 9-Cbz |
| | | | DBF | | |
| 124213 | 9-Cbz | 9-Cbz | 2- | 9-Cbz | 9-Cbz |
| | | | DBT | | |
| 124214 | 9-Cbz | 9-Cbz | 4-t- | 9-Cbz | 9-Cbz |
| | | | BuPh | | |
| 124215 | 9-Cbz | 9-Cbz | 1,3- | 9-Cbz | 9-Cbz |
| | | | di-t- | | |
| | | | BuPh | | |
| 124216 | 9-Cbz | 9-Cbz | 9-Cbz | 9-Cbz | 9-Cbz |
| 124217 | 9-Cbz | 9-Cbz | t-Bu | 4-t- | 4-t- |
| | | | | BuPh | BuPh |
| 124218 | 9-Cbz | 9-Cbz | Ph | 4-t- | 4-t- |
| | | | | BuPh | BuPh |
| 124219 | 9-Cbz | 9-Cbz | CN | 4-t- | 4-t- |
| | | | | BuPh | BuPh |
| 124220 | 9-Cbz | 9-Cbz | DPA | 4-t- | 4-t- |
| | | | | BuPh | BuPh |
| 124221 | 9-Cbz | 9-Cbz | 2- | 4-t- | 4-t- |
| | | | DBF | BuPh | BuPh |
| 124222 | 9-Cbz | 9-Cbz | 2- | 4-t- | 4-t- |
| | | | DBT | BuPh | BuPh |
| 124223 | 9-Cbz | 9-Cbz | 4-t- | 4-t- | 4-t- |
| | | | BuPh | BuPh | BuPh |
| 124224 | 9-Cbz | 9-Cbz | 1,3- | 4-t- | 4-t- |
| | | | di-t- | BuPh | BuPh |
| | | | BuPh | | |
| 124225 | 9-Cbz | 9-Cbz | 9-Cbz | 4-t- | 4-t- |
| | | | | BuPh | BuPh |
| 124226 | 9-Cbz | 9-Cbz | t-Bu | 1,3- | 1,3- |
| | | | | di-t- | di-t- |
| | | | | BuPh | BuPh |
| 124227 | 9-Cbz | 9-Cbz | Ph | 1,3- | 1,3- |
| | | | | di-t- | di-t- |
| | | | | BuPh | BuPh |
| 124228 | 9-Cbz | 9-Cbz | CN | 1,3- | 1,3- |
| | | | | di-t- | di-t- |
| | | | | BuPh | BuPh |
| 124229 | 9-Cbz | 9-Cbz | DPA | 1,3- | 1,3- |
| | | | | di-t- | di-t- |
| | | | | BuPh | BuPh |
| 124230 | 9-Cbz | 9-Cbz | 2- | 1,3- | 1,3- |
| | | | DBF | di-t- | di-t- |
| | | | | BuPh | BuPh |
| 124231 | 9-Cbz | 9-Cbz | 2- | 1,3- | 1,3- |
| | | | DBT | di-t- | di-t- |
| | | | | BuPh | BuPh |
| 124232 | 9-Cbz | 9-Cbz | 4-t- | 1,3- | 1,3- |
| | | | BuPh | di-t- | di-t- |
| | | | | BuPh | BuPh |
| 124233 | 9-Cbz | 9-Cbz | 1,3- | 1,3- | 1,3- |
| | | | di-t- | di-t- | di-t- |
| | | | BuPh | BuPh | BuPh |
| 124234 | 9-Cbz | 9-Cbz | 9-Cbz | 1,3- | 1,3- |
| | | | | di-t- | di-t- |
| | | | | BuPh | BuPh |
| 124235 | CN | CN | t-Bu | H | H |
| 124236 | CN | CN | Ph | H | H |
| 124237 | CN | CN | DPA | H | H |
| 124238 | CN | CN | 2- | H | H |
| | | | DBF | | |
| 124239 | CN | CN | 2- | H | H |
| | | | DBT | | |
| 124240 | CN | CN | 4-t- | H | H |
| | | | BuPh | | |
| 124241 | CN | CN | 1,3- | H | H |
| | | | di-t- | | |
| | | | BuPh | | |
| 124242 | CN | CN | 9-Cbz | H | H |
| 124243 | CN | CN | t-Bu | t-Bu | t-Bu |
| 124244 | CN | CN | Ph | t-Bu | t-Bu |
| 124245 | CN | CN | CN | t-Bu | t-Bu |
| 124246 | CN | CN | DPA | t-Bu | t-Bu |
| 124247 | CN | CN | 2- | t-Bu | t-Bu |
| | | | DBF | | |
| 124248 | CN | CN | 2- | t-Bu | t-Bu |
| | | | DBT | | |
| 124249 | CN | CN | 4-t- | t-Bu | t-Bu |
| | | | BuPh | | |
| 124250 | CN | CN | 1,3- | t-Bu | t-Bu |
| | | | di-t- | | |
| | | | BuPh | | |
| 124251 | CN | CN | 9-Cbz | t-Bu | t-Bu |
| 124252 | CN | CN | t-Bu | Ph | Ph |
| 124253 | CN | CN | Ph | Ph | Ph |
| 124254 | CN | CN | CN | Ph | Ph |
| 124255 | CN | CN | DPA | Ph | Ph |
| 124256 | CN | CN | 2- | Ph | Ph |
| | | | DBF | | |
| 124257 | CN | CN | 2- | Ph | Ph |
| | | | DBT | | |
| 124258 | CN | CN | 4-t- | Ph | Ph |
| | | | BuPh | | |
| 124259 | CN | CN | 1,3- | Ph | Ph |
| | | | di-t- | | |
| | | | BuPh | | |
| 124260 | CN | CN | 9-Cbz | Ph | Ph |
| 124261 | CN | CN | t-Bu | CN | CN |
| 124262 | CN | CN | Ph | CN | CN |
| 124263 | CN | CN | CN | CN | CN |
| 124264 | CN | CN | DPA | CN | CN |
| 124265 | CN | CN | 2- | CN | CN |
| | | | DBF | | |
| 124266 | CN | CN | 2- | CN | CN |
| | | | DBT | | |
| 124267 | CN | CN | 4-t- | CN | CN |
| | | | BuPh | | |
| 124268 | CN | CN | 1,3- | CN | CN |
| | | | di-t- | | |
| | | | BuPh | | |
| 124269 | CN | CN | 9-Cbz | CN | CN |
| 124270 | CN | CN | t-Bu | 9-Cbz | 9-Cbz |
| 124271 | CN | CN | Ph | 9-Cbz | 9-Cbz |
| 124272 | CN | CN | CN | 9-Cbz | 9-Cbz |
| 124273 | CN | CN | DPA | 9-Cbz | 9-Cbz |
| 124274 | CN | CN | 2- | 9-Cbz | 9-Cbz |
| | | | DBF | | |
| 124275 | CN | CN | 2- | 9-Cbz | 9-Cbz |
| | | | DBT | | |
| 124276 | CN | CN | 4-t- | 9-Cbz | 9-Cbz |
| | | | BuPh | | |
| 124277 | CN | CN | 1,3- | 9-Cbz | 9-Cbz |
| | | | di-t- | | |
| | | | BuPh | | |
| 124278 | CN | CN | 9-Cbz | 9-Cbz | 9-Cbz |
| 124279 | CN | CN | t-Bu | 4-t- | 4-t- |
| | | | | BuPh | BuPh |
| 124280 | CN | CN | Ph | 4-t- | 4-t- |
| | | | | BuPh | BuPh |
| 124281 | CN | CN | CN | 4-t- | 4-t- |
| | | | | BuPh | BuPh |
| 124282 | CN | CN | DPA | 4-t- | 4-t- |
| | | | | BuPh | BuPh |
| 124283 | CN | CN | 2- | 4-t- | 4-t- |
| | | | DBF | BuPh | BuPh |
| 124284 | CN | CN | 2- | 4-t- | 4-t- |
| | | | DBT | BuPh | BuPh |
| 124285 | CN | CN | 4-t- | 4-t- | 4-t- |
| | | | BuPh | BuPh | BuPh |
| 124286 | CN | CN | 1,3- | 4-t- | 4-t- |
| | | | di-t- | BuPh | BuPh |
| | | | BuPh | | |
| 124287 | CN | CN | 9-Cbz | 4-t- | 4-t- |
| | | | | BuPh | BuPh |
| 124288 | CN | CN | t-Bu | 1,3- | 1,3- |
| | | | | di-t- | di-t- |
| | | | | BuPh | BuPh |
| 124289 | CN | CN | Ph | 1,3- | 1,3- |
| | | | | di-t- | di-t- |
| | | | | BuPh | BuPh |
| 124290 | CN | CN | CN | 1,3- | 1,3- |
| | | | | di-t- | di-t- |
| | | | | BuPh | BuPh |
| 124291 | CN | CN | DPA | 1,3- | 1,3- |
| | | | | di-t- | di-t- |
| | | | | BuPh | BuPh |
| 124292 | CN | CN | 2- | 1,3- | 1,3- |
| | | | DBF | di-t- | di-t- |
| | | | | BuPh | BuPh |
| 124293 | CN | CN | 2- | 1,3- | 1,3- |
| | | | DBT | di-t- | di-t- |
| | | | | BuPh | BuPh |
| 124294 | CN | CN | 4-t- | 1,3- | 1,3- |
| | | | BuPh | di-t- | di-t- |
| | | | | BuPh | BuPh |
| 124295 | CN | CN | 1,3- | 1,3- | 1,3- |
| | | | di-t- | di-t- | di-t- |
| | | | BuPh | BuPh | BuPh |
| 124296 | CN | CN | 9-Cbz | 1,3- | 1,3- |
| | | | | di-t- | di-t- |
| | | | | BuPh | BuPh |
| 124297 | 4-t- | 4-t- | t-Bu | H | H |
| | BuPh | BuPh | | | |
| 124298 | 4-t- | 4-t- | Ph | H | H |
| | BuPh | BuPh | | | |
| 124299 | 4-t- | 4-t- | CN | H | H |
| | BuPh | BuPh | | | |
| 124300 | 4-t- | 4-t- | DPA | H | H |
| | BuPh | BuPh | | | |
| 124301 | 4-t- | 4-t- | 2- | H | H |
| | BuPh | BuPh | DBF | | |
| 124302 | 4-t- | 4-t- | 2- | H | H |
| | BuPh | BuPh | DBT | | |
| 124303 | 4-t- | 4-t- | 1,3- | H | H |
| | BuPh | BuPh | di-t- | | |
| | | | BuPh | | |
| 124304 | 4-t- | 4-t- | 9-Cbz | H | H |
| | BuPh | BuPh | | | |
| 124305 | 4-t- | 4-t- | t-Bu | t-Bu | t-Bu |
| | BuPh | BuPh | | | |
| 124306 | 4-t- | 4-t- | Ph | t-Bu | t-Bu |
| | BuPh | BuPh | | | |
| 124307 | 4-t- | 4-t- | CN | t-Bu | t-Bu |
| | BuPh | BuPh | | | |
| 124308 | 4-t- | 4-t- | DPA | t-Bu | t-Bu |
| | BuPh | BuPh | | | |
| 124309 | 4-t- | 4-t- | 2- | t-Bu | t-Bu |
| | BuPh | BuPh | DBF | | |
| 124310 | 4-t- | 4-t- | 2- | t-Bu | t-Bu |
| | BuPh | BuPh | DBT | | |
| 124311 | 4-t- | 4-t- | 4-t- | t-Bu | t-Bu |
| | BuPh | BuPh | BuPh | | |
| 124312 | 4-t- | 4-t- | 1,3- | t-Bu | t-Bu |
| | BuPh | BuPh | di-t- | | |
| | | | BuPh | | |
| 124313 | 4-t- | 4-t- | 9-Cbz | t-Bu | t-Bu |
| | BuPh | BuPh | | | |
| 124314 | 4-t- | 4-t- | t-Bu | Ph | Ph |
| | BuPh | BuPh | | | |
| 124315 | 4-t- | 4-t- | Ph | Ph | Ph |
| | BuPh | BuPh | | | |
| 124316 | 4-t- | 4-t- | CN | Ph | Ph |
| | BuPh | BuPh | | | |
| 124317 | 4-t- | 4-t- | DPA | Ph | Ph |
| | BuPh | BuPh | | | |
| 124318 | 4-t- | 4-t- | 2- | Ph | Ph |
| | BuPh | BuPh | DBF | | |
| 124319 | 4-t- | 4-t- | 2- | Ph | Ph |
| | BuPh | BuPh | DBT | | |
| 124320 | 4-t- | 4-t- | 4-t- | Ph | Ph |
| | BuPh | BuPh | BuPh | | |
| 124321 | 4-t- | 4-t- | 1,3- | Ph | Ph |
| | BuPh | BuPh | di-t- | | |
| | | | BuPh | | |
| 124322 | 4-t- | 4-t- | 9-Cbz | Ph | Ph |
| | BuPh | BuPh | | | |
| 124323 | 4-t- | 4-t- | t-Bu | CN | CN |
| | BuPh | BuPh | | | |
| 124324 | 4-t- | 4-t- | Ph | CN | CN |
| | BuPh | BuPh | | | |
| 124325 | 4-t- | 4-t- | CN | CN | CN |
| | BuPh | BuPh | | | |
| 124326 | 4-t- | 4-t- | DPA | CN | CN |
| | BuPh | BuPh | | | |
| 124327 | 4-t- | 4-t- | 2- | CN | CN |
| | BuPh | BuPh | DBF | | |
| 124328 | 4-t- | 4-t- | 2- | CN | CN |
| | BuPh | BuPh | DBT | | |
| 124329 | 4-t- | 4-t- | 4-t- | CN | CN |
| | BuPh | BuPh | BuPh | | |
| 124330 | 4-t- | 4-t- | 1,3- | CN | CN |
| | BuPh | BuPh | di-t- | | |
| | | | BuPh | | |
| 124331 | 4-t- | 4-t- | 9-Cbz | CN | CN |
| | BuPh | BuPh | | | |
| 124332 | 4-t- | 4-t- | t-Bu | 9-Cbz | 9-Cbz |
| | BuPh | BuPh | | | |
| 124333 | 4-t- | 4-t- | Ph | 9-Cbz | 9-Cbz |
| | BuPh | BuPh | | | |
| 124334 | 4-t- | 4-t- | CN | 9-Cbz | 9-Cbz |
| | BuPh | BuPh | | | |
| 124335 | 4-t- | 4-t- | DPA | 9-Cbz | 9-Cbz |
| | BuPh | BuPh | | | |
| 124336 | 4-t- | 4-t- | 2- | 9-Cbz | 9-Cbz |
| | BuPh | BuPh | DBF | | |
| 124337 | 4-t- | 4-t- | 2- | 9-Cbz | 9-Cbz |
| | BuPh | BuPh | DBT | | |
| 124338 | 4-t- | 4-t- | 4-t- | 9-Cbz | 9-Cbz |
| | BuPh | BuPh | BuPh | | |
| 124339 | 4-t- | 4-t- | 1,3- | 9-Cbz | 9-Cbz |
| | BuPh | BuPh | di-t- | | |
| | | | BuPh | | |
| 124340 | 4-t- | 4-t- | 9-Cbz | 9-Cbz | 9-Cbz |
| | BuPh | BuPh | | | |
| 124341 | 4-t- | 4-t- | t-Bu | 4-t- | 4-t- |
| | BuPh | BuPh | | BuPh | BuPh |
| 124342 | 4-t- | 4-t- | Ph | 4-t- | 4-t- |
| | BuPh | BuPh | | BuPh | BuPh |
| 124343 | 4-t- | 4-t- | CN | 4-t- | 4-t- |
| | BuPh | BuPh | | BuPh | BuPh |
| 124344 | 4-t- | 4-t- | DPA | 4-t- | 4-t- |
| | BuPh | BuPh | | BuPh | BuPh |
| 124345 | 4-t- | 4-t- | 2- | 4-t- | 4-t- |
| | BuPh | BuPh | DBF | BuPh | BuPh |
| 124346 | 4-t- | 4-t- | 2- | 4-t- | 4-t- |
| | BuPh | BuPh | DBT | BuPh | BuPh |
| 124347 | 4-t- | 4-t- | 4-t- | 4-t- | 4-t- |
| | BuPh | BuPh | BuPh | BuPh | BuPh |
| 124348 | 4-t- | 4-t- | 1,3- | 4-t- | 4-t- |
| | BuPh | BuPh | di-t- | BuPh | BuPh |
| | | | BuPh | | |
| 124349 | 4-t- | 4-t- | 9-Cbz | 4-t- | 4-t- |
| | BuPh | BuPh | | BuPh | BuPh |
| 124350 | 4-t- | 4-t- | t-Bu | 1,3- | 1,3- |
| | BuPh | BuPh | | di-t- | di-t- |
| | | | | BuPh | BuPh |
| 124351 | 4-t- | 4-t- | Ph | 1,3- | 1,3- |
| | BuPh | BuPh | | di-t- | di-t- |
| | | | | BuPh | BuPh |
| 124352 | 4-t- | 4-t- | CN | 1,3- | 1,3- |
| | BuPh | BuPh | | di-t- | di-t- |
| | | | | BuPh | BuPh |
| 124353 | 4-t- | 4-t- | DPA | 1,3- | 1,3- |
| | BuPh | BuPh | | di-t- | di-t- |
| | | | | BuPh | BuPh |
| 124354 | 4-t- | 4-t- | 2- | 1,3- | 1,3- |
| | BuPh | BuPh | DBF | di-t- | di-t- |
| | | | | BuPh | BuPh |
| 124355 | 4-t- | 4-t- | 2- | 1,3- | 1,3- |
| | BuPh | BuPh | DBT | di-t- | di-t- |
| | | | | BuPh | BuPh |
| 124356 | 4-t- | 4-t- | 4-t- | 1,3- | 1,3- |
| | BuPh | BuPh | BuPh | di-t- | di-t- |
| | | | | BuPh | BuPh |
| 124357 | 4-t- | 4-t- | 1,3- | 1,3- | 1,3- |
| | BuPh | BuPh | di-t- | di-t- | di-t- |
| | | | BuPh | BuPh | BuPh |
| 124358 | 4-t- | 4-t- | 9-Cbz | 1,3- | 1,3- |
| | BuPh | BuPh | | di-t- | di-t- |
| | | | | BuPh | BuPh |
| 124359 | 1,3- | 1,3- | t-Bu | H | H |
| | di-t- | di-t- | | | |
| | BuPh | BuPh | | | |
| 124360 | 1,3- | 1,3- | Ph | H | H |
| | di-t- | di-t- | | | |
| | BuPh | BuPh | | | |
| 124361 | 1,3- | 1,3- | CN | H | H |
| | di-t- | di-t- | | | |
| | BuPh | BuPh | | | |
| 124362 | 1,3- | 1,3- | DPA | H | H |
| | di-t- | di-t- | | | |
| | BuPh | BuPh | | | |
| 124363 | 1,3- | 1,3- | 2- | H | H |
| | di-t- | di-t- | DBF | | |
| | BuPh | BuPh | | | |
| 124364 | 1,3- | 1,3- | 2- | H | H |
| | di-t- | di-t- | DBT | | |
| | BuPh | BuPh | | | |
| 124365 | 1,3- | 1,3- | 4-t- | H | H |
| | di-t- | di-t- | BuPh | | |
| | BuPh | BuPh | | | |
| 124366 | 1,3- | 1,3- | 9-Cbz | H | H |
| | di-t- | di-t- | | | |
| | BuPh | BuPh | | | |
| 124367 | 1,3- | 1,3- | t-Bu | t-Bu | t-Bu |
| | di-t- | di-t- | | | |
| | BuPh | BuPh | | | |
| 124368 | 1,3- | 1,3- | Ph | t-Bu | t-Bu |
| | di-t- | di-t- | | | |
| | BuPh | BuPh | | | |
| 124369 | 1,3- | 1,3- | CN | t-Bu | t-Bu |
| | di-t- | di-t- | | | |
| | BuPh | BuPh | | | |
| 124370 | 1,3- | 1,3- | DPA | t-Bu | t-Bu |
| | di-t- | di-t- | | | |
| | BuPh | BuPh | | | |
| 124371 | 1,3- | 1,3- | 2- | t-Bu | t-Bu |
| | di-t- | di-t- | DBF | | |
| | BuPh | BuPh | | | |
| 124372 | 1,3- | 1,3- | 2- | t-Bu | t-Bu |
| | di-t- | di-t- | DBT | | |
| | BuPh | BuPh | | | |
| 124373 | 1,3- | 1,3- | 4-t- | t-Bu | t-Bu |
| | di-t- | di-t- | BuPh | | |
| | BuPh | BuPh | | | |
| 124374 | 1,3- | 1,3- | 1,3- | t-Bu | t-Bu |
| | di-t- | di-t- | di-t- | | |
| | BuPh | BuPh | BuPh | | |
| 124375 | 1,3- | 1,3- | 9-Cbz | t-Bu | t-Bu |
| | di-t- | di-t- | | | |
| | BuPh | BuPh | | | |
| 124376 | 1,3- | 1,3- | t-Bu | Ph | Ph |
| | di-t- | di-t- | | | |
| | BuPh | BuPh | | | |
| 124377 | 1,3- | 1,3- | Ph | Ph | Ph |
| | di-t- | di-t- | | | |
| | BuPh | BuPh | | | |
| 124378 | 1,3- | 1,3- | CN | Ph | Ph |
| | di-t- | di-t- | | | |
| | BuPh | BuPh | | | |
| 124379 | 1,3- | 1,3- | DPA | Ph | Ph |
| | di-t- | di-t- | | | |
| | BuPh | BuPh | | | |
| 124380 | 1,3- | 1,3- | 2- | Ph | Ph |
| | di-t- | di-t- | DBF | | |
| | BuPh | BuPh | | | |
| 124381 | 1,3- | 1,3- | 2- | Ph | Ph |
| | di-t- | di-t- | DBT | | |
| | BuPh | BuPh | | | |
| 124382 | 1,3- | 1,3- | 4-t- | Ph | Ph |
| | di-t- | di-t- | BuPh | | |
| | BuPh | BuPh | | | |
| 124383 | 1,3- | 1,3- | 1,3- | Ph | Ph |
| | di-t- | di-t- | di-t- | | |
| | BuPh | BuPh | BuPh | | |
| 124384 | 1,3- | 1,3- | 9-Cbz | Ph | Ph |
| | di-t- | di-t- | | | |
| | BuPh | BuPh | | | |
| 124385 | 1,3- | 1,3- | t-Bu | CN | CN |
| | di-t- | di-t- | | | |
| | BuPh | BuPh | | | |
| 124386 | 1,3- | 1,3- | Ph | CN | CN |
| | di-t- | di-t- | | | |
| | BuPh | BuPh | | | |
| 124387 | 1,3- | 1,3- | CN | CN | CN |
| | di-t- | di-t- | | | |
| | BuPh | BuPh | | | |
| 124388 | 1,3- | 1,3- | DPA | CN | CN |
| | di-t- | di-t- | | | |
| | BuPh | BuPh | | | |
| 124389 | 1,3- | 1,3- | 2- | CN | CN |
| | di-t- | di-t- | DBF | | |
| | BuPh | BuPh | | | |
| 124390 | 1,3- | 1,3- | 2- | CN | CN |
| | di-t- | di-t- | DBT | | |
| | BuPh | BuPh | | | |
| 124391 | 1,3- | 1,3- | 4-t- | CN | CN |
| | di-t- | di-t- | BuPh | | |
| | BuPh | BuPh | | | |
| 124392 | 1,3- | 1,3- | 1,3- | CN | CN |
| | di-t- | di-t- | di-t- | | |
| | BuPh | BuPh | BuPh | | |
| 124393 | 1,3- | 1,3- | 9-Cbz | CN | CN |
| | di-t- | di-t- | | | |
| | BuPh | BuPh | | | |
| 124394 | 1,3- | 1,3- | t-Bu | 9-Cbz | 9-Cbz |
| | di-t- | di-t- | | | |
| | BuPh | BuPh | | | |
| 124395 | 1,3- | 1,3- | Ph | 9-Cbz | 9-Cbz |
| | di-t- | di-t- | | | |
| | BuPh | BuPh | | | |
| 124396 | 1,3- | 1,3- | CN | 9-Cbz | 9-Cbz |
| | di-t- | di-t- | | | |
| | BuPh | BuPh | | | |
| 124397 | 1,3- | 1,3- | DPA | 9-Cbz | 9-Cbz |
| | di-t- | di-t- | | | |
| | BuPh | BuPh | | | |
| 124398 | 1,3- | 1,3- | 2- | 9-Cbz | 9-Cbz |
| | di-t- | di-t- | DBF | | |
| | BuPh | BuPh | | | |
| 124399 | 1,3- | 1,3- | 2- | 9-Cbz | 9-Cbz |
| | di-t- | di-t- | DBT | | |
| | BuPh | BuPh | | | |
| 124400 | 1,3- | 1,3- | 4-t- | 9-Cbz | 9-Cbz |
| | di-t- | di-t- | BuPh | | |
| | BuPh | BuPh | | | |
| 124401 | 1,3- | 1,3- | 1,3- | 9-Cbz | 9-Cbz |
| | di-t- | di-t- | di-t- | | |
| | BuPh | BuPh | BuPh | | |
| 124402 | 1,3- | 1,3- | 9-Cbz | 9-Cbz | 9-Cbz |
| | di-t- | di-t- | | | |
| | BuPh | BuPh | | | |
| 124403 | 1,3- | 1,3- | t-Bu | 4-t- | 4-t- |
| | di-t- | di-t- | | BuPh | BuPh |
| | BuPh | BuPh | | | |
| 124404 | 1,3- | 1,3- | Ph | 4-t- | 4-t- |
| | di-t- | di-t- | | BuPh | BuPh |
| | BuPh | BuPh | | | |
| 124405 | 1,3- | 1,3- | CN | 4-t- | 4-t- |
| | di-t- | di-t- | | BuPh | BuPh |
| | BuPh | BuPh | | | |
| 124406 | 1,3- | 1,3- | DPA | 4-t- | 4-t- |
| | di-t- | di-t- | | BuPh | BuPh |
| | BuPh | BuPh | | | |
| 124407 | 1,3- | 1,3- | 2- | 4-t- | 4-t- |
| | di-t- | di-t- | DBF | BuPh | BuPh |
| | BuPh | BuPh | | | |
| 124408 | 1,3- | 1,3- | 2- | 4-t- | 4-t- |
| | di-t- | di-t- | DBT | BuPh | BuPh |
| | BuPh | BuPh | | | |
| 124409 | 1,3- | 1,3- | 4-t- | 4-t- | 4-t- |
| | di-t- | di-t- | BuPh | BuPh | BuPh |
| | BuPh | BuPh | | | |
| 124410 | 1,3- | 1,3- | 1,3- | 4-t- | 4-t- |
| | di-t- | di-t- | di-t- | BuPh | BuPh |
| | BuPh | BuPh | BuPh | | |
| 124411 | 1,3- | 1,3- | 9-Cbz | 4-t- | 4-t- |
| | di-t- | di-t- | | BuPh | BuPh |
| | BuPh | BuPh | | | |
| 124412 | 1,3- | 1,3- | t-Bu | 1,3- | 1,3- |
| | di-t- | di-t- | | di-t- | di-t- |
| | BuPh | BuPh | | BuPh | BuPh |
| 124413 | 1,3- | 1,3- | Ph | 1,3- | 1,3- |
| | di-t- | di-t- | | di-t- | di-t- |
| | BuPh | BuPh | | BuPh | BuPh |
| 124414 | 1,3- | 1,3- | CN | 1,3- | 1,3- |
| | di-t- | di-t- | | di-t- | di-t- |
| | BuPh | BuPh | | BuPh | BuPh |
| 124415 | 1,3- | 1,3- | DPA | 1,3- | 1,3- |
| | di-t- | di-t- | | di-t- | di-t- |
| | BuPh | BuPh | | BuPh | BuPh |
| 124416 | 1,3- | 1,3- | 2- | 1,3- | 1,3- |
| | di-t- | di-t- | DBF | di-t- | di-t- |
| | BuPh | BuPh | | BuPh | BuPh |
| 124417 | 1,3- | 1,3- | 2- | 1,3- | 1,3- |
| | di-t- | di-t- | DBT | di-t- | di-t- |
| | BuPh | BuPh | | BuPh | BuPh |
| 124418 | 1,3- | 1,3- | 4-t- | 1,3- | 1,3- |
| | di-t- | di-t- | BuPh | di-t- | di-t- |
| | BuPh | BuPh | | BuPh | BuPh |
| 124419 | 1,3- | 1,3- | 1,3- | 1,3- | 1,3- |
| | di-t- | di-t- | di-t- | di-t- | di-t- |
| | BuPh | BuPh | BuPh | BuPh | BuPh |
| 124420 | 1,3- | 1,3- | 9-Cbz | 1,3- | 1,3- |
| | di-t- | di-t- | | di-t- | di-t- |
| | BuPh | BuPh | | BuPh | BuPh |

### Group HC6

The heterocyclic compound may be represented by Formula 14-1, and substituents may be defined as shown in Table 6.

TABLE 6

| Compound | | | | | |
|---|---|---|---|---|---|
| No. | R_{11b} | R_{12c} | R_{15C} | R_{18b} | R_{19b} |
| 141001 | t-Bu | t-Bu | t-Bu | t-Bu | t-Bu |
| 141002 | Ph | Ph | t-Bu | Ph | Ph |
| 141003 | t-Bu | t-Bu | H | H | H |
| 141004 | H | H | t-Bu | H | H |
| 141005 | H | H | H | t-Bu | t-Bu |
| 141006 | t-Bu | t-Bu | t-Bu | H | H |
| 141007 | H | H | t-Bu | t-Bu | t-Bu |
| 141008 | t-Bu | t-Bu | H | t-Bu | t-Bu |
| 141009 | Ph | Ph | H | H | H |
| 141010 | H | H | Ph | H | H |
| 141011 | H | H | H | Ph | Ph |
| 141012 | Ph | Ph | Ph | H | H |
| 141013 | H | H | Ph | Ph | Ph |
| 141014 | Ph | Ph | H | Ph | Ph |
| 141015 | CN | CN | H | H | H |
| 141016 | H | H | CN | H | H |
| 141017 | H | H | H | CN | CN |
| 141018 | CN | CN | CN | H | H |
| 141019 | H | H | CN | CN | CN |
| 141020 | CN | CN | H | CN | CN |
| 141021 | 9-Cbz | 9-Cbz | H | H | H |
| 141022 | H | H | 9-Cbz | H | H |
| 141023 | H | H | H | 9-Cbz | 9-Cbz |
| 141024 | 9-Cbz | 9-Cbz | 9-Cbz | H | H |
| 141025 | H | H | 9-Cbz | 9-Cbz | 9-Cbz |
| 141026 | 9-Cbz | 9-Cbz | H | 9-Cbz | 9-Cbz |
| 141027 | Me | Me | H | H | H |
| 141028 | H | H | Me | H | H |
| 141029 | H | H | H | Me | Me |
| 141030 | Me | Me | Me | H | H |
| 141031 | H | H | Me | Me | Me |
| 141032 | Me | Me | H | Me | Me |
| 141033 | CD₃ | CD3 | H | H | H |
| 141034 | H | H | CD3 | H | H |
| 141035 | H | H | H | CD3 | CD3 |
| 141036 | CD3 | CD3 | CD3 | H | H |
| 141037 | H | H | CD3 | CD3 | CD3 |
| 141038 | CD3 | CD3 | H | CD3 | CD3 |
| 141039 | 4-t- | 4-t- | H | H | H |
| | BuPh | BuPh | | | |
| 141040 | H | H | 4-t- | H | H |
| | | | BuPh | | |
| 141041 | H | H | H | 4-t- | 4-t- |
| | | | | BuPh | BuPh |
| 141042 | 4-t- | 4-t- | 4-t- | H | H |
| | BuPh | BuPh | BuPh | | |
| 141043 | H | H | 4-t- | 4-t- | 4-t- |
| | | | BuPh | BuPh | BuPh |
| 141044 | 4-t- | 4-t- | H | 4-t- | 4-t- |
| | BuPh | BuPh | | BuPh | BuPh |
| 141045 | 1,3- | 1,3- | H | H | H |
| | di-t- | di-t- | | | |
| | BuPh | BuPh | | | |
| 141046 | H | H | 1,3- | H | H |
| | | | di-t- | | |
| | | | BuPh | | |
| 141047 | H | H | H | 1,3- | 1,3- |
| | | | | di-t- | di-t- |
| | | | | BuPh | BuPh |
| 141048 | 1,3- | 1,3- | 1,3- | H | H |
| | di-t- | di-t- | di-t- | | |
| | BuPh | BuPh | BuPh | | |
| 141049 | H | H | 1,3- | 1,3- | 1,3- |
| | | | di-t- | di-t- | di-t- |
| | | | BuPh | BuPh | BuPh |
| 141050 | 1,3- | 1,3- | H | 1,3- | 1,3- |
| | di-t- | di-t- | | di-t- | di-t- |
| | BuPh | BuPh | | BuPh | BuPh |
| 141051 | t-Bu | t-Bu | Ph | H | H |
| 141052 | t-Bu | t-Bu | CN | H | H |
| 141053 | t-Bu | t-Bu | DPA | H | H |
| 141054 | t-Bu | t-Bu | 2- | H | H |
| | | | DBF | | |
| 141055 | t-Bu | t-Bu | 2- | H | H |
| | | | DBT | | |
| 141056 | t-Bu | t-Bu | 4-t- | H | H |
| | | | BuPh | | |
| 141057 | t-Bu | t-Bu | 1,3- | H | H |
| | | | di-t- | | |
| | | | BuPh | | |
| 141058 | t-Bu | t-Bu | 9-Cbz | H | H |
| 141059 | t-Bu | t-Bu | Ph | t-Bu | t-Bu |
| 141060 | t-Bu | t-Bu | CN | t-Bu | t-Bu |
| 141061 | t-Bu | t-Bu | DPA | t-Bu | t-Bu |
| 141062 | t-Bu | t-Bu | 2- | t-Bu | t-Bu |
| | | | DBF | | |
| 141063 | t-Bu | t-Bu | 2- | t-Bu | t-Bu |
| | | | DBT | | |
| 141064 | t-Bu | t-Bu | 4-t- | t-Bu | t-Bu |
| | | | BuPh | | |
| 141065 | t-Bu | t-Bu | 1,3- | t-Bu | t-Bu |
| | | | di-t- | | |
| | | | BuPh | | |
| 141066 | t-Bu | t-Bu | 9-Cbz | t-Bu | t-Bu |
| 141067 | t-Bu | t-Bu | t-Bu | Ph | Ph |
| 141068 | t-Bu | t-Bu | Ph | Ph | Ph |
| 141069 | t-Bu | t-Bu | CN | Ph | Ph |
| 141070 | t-Bu | t-Bu | DPA | Ph | Ph |
| 141071 | t-Bu | t-Bu | 2- | Ph | Ph |
| | | | DBF | | |
| 141072 | t-Bu | t-Bu | 2- | Ph | Ph |
| | | | DBT | | |
| 141073 | t-Bu | t-Bu | 4-t- | Ph | Ph |
| | | | BuPh | | |
| 141074 | t-Bu | t-Bu | 1,3- | Ph | Ph |
| | | | di-t- | | |
| | | | BuPh | | |
| 141075 | t-Bu | t-Bu | 9-Cbz | Ph | Ph |
| 141076 | t-Bu | t-Bu | t-Bu | CN | CN |
| 141077 | t-Bu | t-Bu | Ph | CN | CN |
| 141078 | t-Bu | t-Bu | CN | CN | CN |
| 141079 | t-Bu | t-Bu | DPA | CN | CN |
| 141080 | t-Bu | t-Bu | 2- | CN | CN |
| | | | DBF | | |
| 141081 | t-Bu | t-Bu | 2- | CN | CN |
| | | | DBT | | |
| 141082 | t-Bu | t-Bu | 4-t- | CN | CN |
| | | | BuPh | | |
| 141083 | t-Bu | t-Bu | 1,3- | CN | CN |
| | | | di-t- | | |
| | | | BuPh | | |
| 141084 | t-Bu | t-Bu | 9-Cbz | CN | CN |
| 141085 | t-Bu | t-Bu | t-Bu | 9-Cbz | 9-Cbz |
| 141086 | t-Bu | t-Bu | Ph | 9-Cbz | 9-Cbz |
| 141087 | t-Bu | t-Bu | CN | 9-Cbz | 9-Cbz |
| 141088 | t-Bu | t-Bu | DPA | 9-Cbz | 9-Cbz |
| 141089 | t-Bu | t-Bu | 2- | 9-Cbz | 9-Cbz |
| | | | DBF | | |
| 141090 | t-Bu | t-Bu | 2- | 9-Cbz | 9-Cbz |
| | | | DBT | | |
| 141091 | t-Bu | t-Bu | 4-t- | 9-Cbz | 9-Cbz |
| | | | BuPh | | |
| 141092 | t-Bu | t-Bu | 1,3- | 9-Cbz | 9-Cbz |
| | | | di-t- | | |
| | | | BuPh | | |
| 141093 | t-Bu | t-Bu | 9-Cbz | 9-Cbz | 9-Cbz |
| 141094 | t-Bu | t-Bu | t-Bu | 4-t- | 4-t- |
| | | | | BuPh | BuPh |
| 141095 | t-Bu | t-Bu | Ph | 4-t- | 4-t- |
| | | | | BuPh | BuPh |
| 141096 | t-Bu | t-Bu | CN | 4-t- | 4-t- |
| | | | | BuPh | BuPh |
| 141097 | t-Bu | t-Bu | DPA | 4-t- | 4-t- |
| | | | | BuPh | BuPh |
| 141098 | t-Bu | t-Bu | 2- | 4-t- | 4-t- |
| | | | DBF | BuPh | BuPh |
| 141099 | t-Bu | t-Bu | 2- | 4-t- | 4-t- |
| | | | DBT | BuPh | BuPh |
| 141100 | t-Bu | t-Bu | 4-t- | 4-t- | 4-t- |
| | | | BuPh | BuPh | BuPh |
| 141101 | t-Bu | t-Bu | 1,3- | 4-t- | 4-t- |
| | | | di-t- | BuPh | BuPh |
| | | | BuPh | | |
| 141102 | t-Bu | t-Bu | 9-Cbz | 4-t- | 4-t- |
| | | | | BuPh | BuPh |
| 141103 | t-Bu | t-Bu | t-Bu | 1,3- | 1,3- |
| | | | | di-t- | di-t- |
| | | | | BuPh | BuPh |
| 141104 | t-Bu | t-Bu | Ph | 1,3- | 1,3- |
| | | | | di-t- | di-t- |
| | | | | BuPh | BuPh |
| 141105 | t-Bu | t-Bu | CN | 1,3- | 1,3- |
| | | | | di-t- | di-t- |
| | | | | BuPh | BuPh |
| 141106 | t-Bu | t-Bu | DPA | 1,3- | 1,3- |
| | | | | di-t- | di-t- |
| | | | | BuPh | BuPh |
| 141107 | t-Bu | t-Bu | 2- | 1,3- | 1,3- |
| | | | DBF | di-t- | di-t- |
| | | | | BuPh | BuPh |
| 141108 | t-Bu | t-Bu | 2- | 1,3- | 1,3- |
| | | | DBT | di-t- | di-t- |
| | | | | BuPh | BuPh |
| 141109 | t-Bu | t-Bu | 4-t- | 1,3- | 1,3- |
| | | | BuPh | di-t- | di-t- |
| | | | | BuPh | BuPh |
| 141110 | t-Bu | t-Bu | 1,3- | 1,3- | 1,3- |
| | | | di-t- | di-t- | di-t- |
| | | | BuPh | BuPh | BuPh |
| 141111 | t-Bu | t-Bu | 9-Cbz | 1,3- | 1,3- |
| | | | | di-t- | di-t- |
| | | | | BuPh | BuPh |
| 141112 | Ph | Ph | t-Bu | H | H |
| 141113 | Ph | Ph | CN | H | H |
| 141114 | Ph | Ph | DPA | H | H |
| 141115 | Ph | Ph | 2- | H | H |
| | | | DBF | | |
| 141116 | Ph | Ph | 2- | H | H |
| | | | DBT | | |
| 141117 | Ph | Ph | 4-t- | H | H |
| | | | BuPh | | |
| 141118 | Ph | Ph | 1,3- | H | H |
| | | | di-t- | | |
| | | | BuPh | | |
| 141119 | Ph | Ph | 9-Cbz | H | H |
| 141120 | Ph | Ph | t-Bu | t-Bu | t-Bu |
| 141121 | Ph | Ph | Ph | t-Bu | t-Bu |
| 141122 | Ph | Ph | CN | t-Bu | t-Bu |
| 141123 | Ph | Ph | DPA | t-Bu | t-Bu |
| 141124 | Ph | Ph | 2- | t-Bu | t-Bu |
| | | | DBF | | |
| 141125 | Ph | Ph | 2- | t-Bu | t-Bu |
| | | | DBT | | |
| 141126 | Ph | Ph | 4-t- | t-Bu | t-Bu |
| | | | BuPh | | |
| 141127 | Ph | Ph | 1,3- | t-Bu | t-Bu |
| | | | di-t- | | |
| | | | BuPh | | |
| 141128 | Ph | Ph | 9-Cbz | t-Bu | t-Bu |
| 141129 | Ph | Ph | Ph | Ph | Ph |
| 141130 | Ph | Ph | CN | Ph | Ph |
| 141131 | Ph | Ph | DPA | Ph | Ph |
| 141132 | Ph | Ph | 2- | Ph | Ph |
| | | | DBF | | |
| 141133 | Ph | Ph | 2- | Ph | Ph |
| | | | DBT | | |
| 141134 | Ph | Ph | 4-t- | Ph | Ph |
| | | | BuPh | | |
| 141135 | Ph | Ph | 1,3- | Ph | Ph |
| | | | di-t- | | |
| | | | BuPh | | |
| 141136 | Ph | Ph | 9-Cbz | Ph | Ph |
| 141137 | Ph | Ph | t-Bu | CN | CN |
| 141138 | Ph | Ph | Ph | CN | CN |
| 141139 | Ph | Ph | CN | CN | CN |
| 141140 | Ph | Ph | DPA | CN | CN |
| 141141 | Ph | Ph | 2- | CN | CN |
| | | | DBF | | |
| 141142 | Ph | Ph | 2- | CN | CN |
| | | | DBT | | |
| 141143 | Ph | Ph | 4-t- | CN | CN |
| | | | BuPh | | |
| 141144 | Ph | Ph | 1,3- | CN | CN |
| | | | di-t- | | |
| | | | BuPh | | |
| 141145 | Ph | Ph | 9-Cbz | CN | CN |
| 141146 | Ph | Ph | t-Bu | 9-Cbz | 9-Cbz |
| 141147 | Ph | Ph | Ph | 9-Cbz | 9-Cbz |
| 141148 | Ph | Ph | CN | 9-Cbz | 9-Cbz |
| 141149 | Ph | Ph | DPA | 9-Cbz | 9-Cbz |
| 141150 | Ph | Ph | 2- | 9-Cbz | 9-Cbz |
| | | | DBF | | |
| 141151 | Ph | Ph | 2- | 9-Cbz | 9-Cbz |
| | | | DBT | | |
| 141152 | Ph | Ph | 4-t- | 9-Cbz | 9-Cbz |
| | | | BuPh | | |
| 141153 | Ph | Ph | 1,3- | 9-Cbz | 9-Cbz |
| | | | di-t- | | |
| | | | BuPh | | |
| 141154 | Ph | Ph | 9-Cbz | 9-Cbz | 9-Cbz |
| 141155 | Ph | Ph | t-Bu | 4-t- | 4-t- |
| | | | | BuPh | BuPh |
| 141156 | Ph | Ph | Ph | 4-t- | 4-t- |
| | | | | BuPh | BuPh |
| 141157 | Ph | Ph | CN | 4-t- | 4-t- |
| | | | | BuPh | BuPh |
| 141158 | Ph | Ph | DPA | 4-t- | 4-t- |
| | | | | BuPh | BuPh |
| 141159 | Ph | Ph | 2- | 4-t- | 4-t- |
| | | | DBF | BuPh | BuPh |
| 141160 | Ph | Ph | 2- | 4-t- | 4-t- |
| | | | DBT | BuPh | BuPh |
| 141161 | Ph | Ph | 4-t- | 4-t- | 4-t- |
| | | | BuPh | BuPh | BuPh |
| 141162 | Ph | Ph | 1,3- | 4-t- | 4-t- |
| | | | di-t- | BuPh | BuPh |
| | | | BuPh | | |
| 141163 | Ph | Ph | 9-Cbz | 4-t- | 4-t- |
| | | | | BuPh | BuPh |
| 141164 | Ph | Ph | t-Bu | 1,3- | 1,3- |
| | | | | di-t- | di-t- |
| | | | | BuPh | BuPh |
| 141165 | Ph | Ph | Ph | 1,3- | 1,3- |
| | | | | di-t- | di-t- |
| | | | | BuPh | BuPh |
| 141166 | Ph | Ph | CN | 1,3- | 1,3- |
| | | | | di-t- | di-t- |
| | | | | BuPh | BuPh |
| 141167 | Ph | Ph | DPA | 1,3- | 1,3- |
| | | | | di-t- | di-t- |
| | | | | BuPh | BuPh |
| 141168 | Ph | Ph | 2- | 1,3- | 1,3- |
| | | | DBF | di-t- | di-t- |
| | | | | BuPh | BuPh |
| 141169 | Ph | Ph | 2- | 1,3- | 1,3- |
| | | | DBT | di-t- | di-t- |
| | | | | BuPh | BuPh |
| 141170 | Ph | Ph | 4-t- | 1,3- | 1,3- |
| | | | BuPh | di-t- | di-t- |
| | | | | BuPh | BuPh |
| 141171 | Ph | Ph | 1,3- | 1,3- | 1,3- |
| | | | di-t- | di-t- | di-t- |
| | | | BuPh | BuPh | BuPh |
| 141172 | Ph | Ph | 9-Cbz | 1,3- | 1,3- |
| | | | | di-t- | di-t- |
| | | | | BuPh | BuPh |
| 141173 | 9-Cbz | 9-Cbz | t-Bu | H | H |
| 141174 | 9-Cbz | 9-Cbz | Ph | H | H |
| 141175 | 9-Cbz | 9-Cbz | CN | H | H |
| 141176 | 9-Cbz | 9-Cbz | DPA | H | H |
| 141177 | 9-Cbz | 9-Cbz | 2- | H | H |
| | | | DBF | | |
| 141178 | 9-Cbz | 9-Cbz | 2- | H | H |
| | | | DBT | | |
| 141179 | 9-Cbz | 9-Cbz | 4-t- | H | H |
| | | | BuPh | | |
| 141180 | 9-Cbz | 9-Cbz | 1,3- | H | H |
| | | | di-t- | | |
| | | | BuPh | | |
| 141181 | 9-Cbz | 9-Cbz | t-Bu | t-Bu | t-Bu |
| 141182 | 9-Cbz | 9-Cbz | Ph | t-Bu | t-Bu |
| 141183 | 9-Cbz | 9-Cbz | CN | t-Bu | t-Bu |
| 141184 | 9-Cbz | 9-Cbz | DPA | t-Bu | t-Bu |
| 141185 | 9-Cbz | 9-Cbz | 2- | t-Bu | t-Bu |
| | | | DBF | | |
| 141186 | 9-Cbz | 9-Cbz | 2- | t-Bu | t-Bu |
| | | | DBT | | |
| 141187 | 9-Cbz | 9-Cbz | 4-t- | t-Bu | t-Bu |
| | | | BuPh | | |
| 141188 | 9-Cbz | 9-Cbz | 1,3- | t-Bu | t-Bu |
| | | | di-t- | | |
| | | | BuPh | | |
| 141189 | 9-Cbz | 9-Cbz | 9-Cbz | t-Bu | t-Bu |
| 141190 | 9-Cbz | 9-Cbz | t-Bu | Ph | Ph |
| 141191 | 9-Cbz | 9-Cbz | Ph | Ph | Ph |
| 141192 | 9-Cbz | 9-Cbz | CN | Ph | Ph |
| 141193 | 9-Cbz | 9-Cbz | DPA | Ph | Ph |
| 141194 | 9-Cbz | 9-Cbz | 2- | Ph | Ph |
| | | | DBF | | |
| 141195 | 9-Cbz | 9-Cbz | 2- | Ph | Ph |
| | | | DBT | | |
| 141196 | 9-Cbz | 9-Cbz | 4-t- | Ph | Ph |
| | | | BuPh | | |
| 141197 | 9-Cbz | 9-Cbz | 1,3- | Ph | Ph |
| | | | di-t- | | |
| | | | BuPh | | |
| 141198 | 9-Cbz | 9-Cbz | 9-Cbz | Ph | Ph |
| 141199 | 9-Cbz | 9-Cbz | t-Bu | CN | CN |
| 141200 | 9-Cbz | 9-Cbz | Ph | CN | CN |
| 141201 | 9-Cbz | 9-Cbz | CN | CN | CN |
| 141202 | 9-Cbz | 9-Cbz | DPA | CN | CN |
| 141203 | 9-Cbz | 9-Cbz | 2- | CN | CN |
| | | | DBF | | |
| 141204 | 9-Cbz | 9-Cbz | 2- | CN | CN |
| | | | DBT | | |
| 141205 | 9-Cbz | 9-Cbz | 4-t- | CN | CN |
| | | | BuPh | | |
| 141206 | 9-Cbz | 9-Cbz | 1,3- | CN | CN |
| | | | di-t- | | |
| | | | BuPh | | |
| 141207 | 9-Cbz | 9-Cbz | 9-Cbz | CN | CN |
| 141208 | 9-Cbz | 9-Cbz | t-Bu | 9-Cbz | 9-Cbz |
| 141209 | 9-Cbz | 9-Cbz | Ph | 9-Cbz | 9-Cbz |
| 141210 | 9-Cbz | 9-Cbz | CN | 9-Cbz | 9-Cbz |
| 141211 | 9-Cbz | 9-Cbz | DPA | 9-Cbz | 9-Cbz |
| 141212 | 9-Cbz | 9-Cbz | 2- | 9-Cbz | 9-Cbz |
| | | | DBF | | |
| 141213 | 9-Cbz | 9-Cbz | 2- | 9-Cbz | 9-Cbz |
| | | | DBT | | |
| 141214 | 9-Cbz | 9-Cbz | 4-t- | 9-Cbz | 9-Cbz |
| | | | BuPh | | |
| 141215 | 9-Cbz | 9-Cbz | 1,3- | 9-Cbz | 9-Cbz |
| | | | di-t- | | |
| | | | BuPh | | |
| 141216 | 9-Cbz | 9-Cbz | 9-Cbz | 9-Cbz | 9-Cbz |
| 141217 | 9-Cbz | 9-Cbz | t-Bu | 4-t- | 4-t- |
| | | | | BuPh | BuPh |
| 141218 | 9-Cbz | 9-Cbz | Ph | 4-t- | 4-t- |
| | | | | BuPh | BuPh |
| 141219 | 9-Cbz | 9-Cbz | CN | 4-t- | 4-t- |
| | | | | BuPh | BuPh |
| 141220 | 9-Cbz | 9-Cbz | DPA | 4-t- | 4-t- |
| | | | | BuPh | BuPh |
| 141221 | 9-Cbz | 9-Cbz | 2- | 4-t- | 4-t- |
| | | | DBF | BuPh | BuPh |
| 141222 | 9-Cbz | 9-Cbz | 2- | 4-t- | 4-t- |
| | | | DBT | BuPh | BuPh |
| 141223 | 9-Cbz | 9-Cbz | 4-t- | 4-t- | 4-t- |
| | | | BuPh | BuPh | BuPh |
| 141224 | 9-Cbz | 9-Cbz | 1,3- | 4-t- | 4-t- |
| | | | di-t- | BuPh | BuPh |
| | | | BuPh | | |
| 141225 | 9-Cbz | 9-Cbz | 9-Cbz | 4-t- | 4-t- |
| | | | | BuPh | BuPh |
| 141226 | 9-Cbz | 9-Cbz | t-Bu | 1,3- | 1,3- |
| | | | | di-t- | di-t- |
| | | | | BuPh | BuPh |
| 141227 | 9-Cbz | 9-Cbz | Ph | 1,3- | 1,3- |
| | | | | di-t- | di-t- |
| | | | | BuPh | BuPh |
| 141228 | 9-Cbz | 9-Cbz | CN | 1,3- | 1,3- |
| | | | | di-t- | di-t- |
| | | | | BuPh | BuPh |
| 141229 | 9-Cbz | 9-Cbz | DPA | 1,3- | 1,3- |
| | | | | di-t- | di-t- |
| | | | | BuPh | BuPh |
| 141230 | 9-Cbz | 9-Cbz | 2- | 1,3- | 1,3- |
| | | | DBF | di-t- | di-t- |
| | | | | BuPh | BuPh |
| 141231 | 9-Cbz | 9-Cbz | 2- | 1,3- | 1,3- |
| | | | DBT | di-t- | di-t- |
| | | | | BuPh | BuPh |
| 141232 | 9-Cbz | 9-Cbz | 4-t- | 1,3- | 1,3- |
| | | | BuPh | di-t- | di-t- |
| | | | | BuPh | BuPh |
| 141233 | 9-Cbz | 9-Cbz | 1,3- | 1,3- | 1,3- |
| | | | di-t- | di-t- | di-t- |
| | | | BuPh | BuPh | BuPh |
| 141234 | 9-Cbz | 9-Cbz | 9-Cbz | 1,3- | 1,3- |
| | | | | di-t- | di-t- |
| | | | | BuPh | BuPh |
| 141235 | CN | CN | t-Bu | H | H |
| 141236 | CN | CN | Ph | H | H |
| 141237 | CN | CN | DPA | H | H |
| 141238 | CN | CN | 2- | H | H |
| | | | DBF | | |
| 141239 | CN | CN | 2- | H | H |
| | | | DBT | | |
| 141240 | CN | CN | 4-t- | H | H |
| | | | BuPh | | |
| 141241 | CN | CN | 1,3- | H | H |
| | | | di-t- | | |
| | | | BuPh | | |
| 141242 | CN | CN | 9-Cbz | H | H |
| 141243 | CN | CN | t-Bu | t-Bu | t-Bu |
| 141244 | CN | CN | Ph | t-Bu | t-Bu |
| 141245 | CN | CN | CN | t-Bu | t-Bu |
| 141246 | CN | CN | DPA | t-Bu | t-Bu |
| 141247 | CN | CN | 2- | t-Bu | t-Bu |
| | | | DBF | | |
| 141248 | CN | CN | 2- | t-Bu | t-Bu |
| | | | DBT | | |
| 141249 | CN | CN | 4-t- | t-Bu | t-Bu |
| | | | BuPh | | |
| 141250 | CN | CN | 1,3- | t-Bu | t-Bu |
| | | | di-t- | | |
| | | | BuPh | | |
| 141251 | CN | CN | 9-Cbz | t-Bu | t-Bu |
| 141252 | CN | CN | t-Bu | Ph | Ph |
| 141253 | CN | CN | Ph | Ph | Ph |
| 141254 | CN | CN | CN | Ph | Ph |
| 141255 | CN | CN | DPA | Ph | Ph |
| 141256 | CN | CN | 2- | Ph | Ph |
| | | | DBF | | |
| 141257 | CN | CN | 2- | Ph | Ph |
| | | | DBT | | |
| 141258 | CN | CN | 4-t- | Ph | Ph |
| | | | BuPh | | |
| 141259 | CN | CN | 1,3- | Ph | Ph |
| | | | di-t- | | |
| | | | BuPh | | |
| 141260 | CN | CN | 9-Cbz | Ph | Ph |
| 141261 | CN | CN | t-Bu | CN | CN |
| 141262 | CN | CN | Ph | CN | CN |
| 141263 | CN | CN | CN | CN | CN |
| 141264 | CN | CN | DPA | CN | CN |
| 141265 | CN | CN | 2- | CN | CN |
| | | | DBF | | |
| 141266 | CN | CN | 2- | CN | CN |
| | | | DBT | | |
| 141267 | CN | CN | 4-t- | CN | CN |
| | | | BuPh | | |
| 141268 | CN | CN | 1,3- | CN | CN |
| | | | di-t- | | |
| | | | BuPh | | |
| 141269 | CN | CN | 9-Cbz | CN | CN |
| 141270 | CN | CN | t-Bu | 9-Cbz | 9-Cbz |
| 141271 | CN | CN | Ph | 9-Cbz | 9-Cbz |
| 141272 | CN | CN | CN | 9-Cbz | 9-Cbz |
| 141273 | CN | CN | DPA | 9-Cbz | 9-Cbz |
| 141274 | CN | CN | 2- | 9-Cbz | 9-Cbz |
| | | | DBF | | |
| 141275 | CN | CN | 2- | 9-Cbz | 9-Cbz |
| | | | DBT | | |
| 141276 | CN | CN | 4-t- | 9-Cbz | 9-Cbz |
| | | | BuPh | | |
| 141277 | CN | CN | 1,3- | 9-Cbz | 9-Cbz |
| | | | di-t- | | |
| | | | BuPh | | |
| 141278 | CN | CN | 9-Cbz | 9-Cbz | 9-Cbz |
| 141279 | CN | CN | t-Bu | 4-t- | 4-t- |
| | | | | BuPh | BuPh |
| 141280 | CN | CN | Ph | 4-t- | 4-t- |
| | | | | BuPh | BuPh |
| 141281 | CN | CN | CN | 4-t- | 4-t- |
| | | | | BuPh | BuPh |
| 141282 | CN | CN | DPA | 4-t- | 4-t- |
| | | | | BuPh | BuPh |
| 141283 | CN | CN | 2- | 4-t- | 4-t- |
| | | | DBF | BuPh | BuPh |
| 141284 | CN | CN | 2- | 4-t- | 4-t- |
| | | | DBT | BuPh | BuPh |
| 141285 | CN | CN | 4-t- | 4-t- | 4-t- |
| | | | BuPh | BuPh | BuPh |
| 141286 | CN | CN | 1,3- | 4-t- | 4-t- |
| | | | di-t- | BuPh | BuPh |
| | | | BuPh | | |
| 141287 | CN | CN | 9-Cbz | 4-t- | 4-t- |
| | | | | BuPh | BuPh |
| 141288 | CN | CN | t-Bu | 1,3- | 1,3- |
| | | | | di-t- | di-t- |
| | | | | BuPh | BuPh |
| 141289 | CN | CN | Ph | 1,3- | 1,3- |
| | | | | di-t- | di-t- |
| | | | | BuPh | BuPh |
| 141290 | CN | CN | CN | 1,3- | 1,3- |
| | | | | di-t- | di-t- |
| | | | | BuPh | BuPh |
| 141291 | CN | CN | DPA | 1,3- | 1,3- |
| | | | | di-t- | di-t- |
| | | | | BuPh | BuPh |
| 141292 | CN | CN | 2- | 1,3- | 1,3- |
| | | | DBF | di-t- | di-t- |
| | | | | BuPh | BuPh |
| 141293 | CN | CN | 2- | 1,3- | 1,3- |
| | | | DBT | di-t- | di-t- |
| | | | | BuPh | BuPh |
| 141294 | CN | CN | 4-t- | 1,3- | 1,3- |
| | | | BuPh | di-t- | di-t- |
| | | | | BuPh | BuPh |
| 141295 | CN | CN | 1,3- | 1,3- | 1,3- |
| | | | di-t- | di-t- | di-t- |
| | | | BuPh | BuPh | BuPh |
| 141296 | CN | CN | 9-Cbz | 1,3- | 1,3- |
| | | | | di-t- | di-t- |
| | | | | BuPh | BuPh |
| 141297 | 4-t- | 4-t- | t-Bu | H | H |
| | BuPh | BuPh | | | |
| 141298 | 4-t- | 4-t- | Ph | H | H |
| | BuPh | BuPh | | | |
| 141299 | 4-t- | 4-t- | CN | H | H |
| | BuPh | BuPh | | | |
| 141300 | 4-t- | 4-t- | DPA | H | H |
| | BuPh | BuPh | | | |
| 141301 | 4-t- | 4-t- | 2- | H | H |
| | BuPh | BuPh | DBF | | |
| 141302 | 4-t- | 4-t- | 2- | H | H |
| | BuPh | BuPh | DBT | | |
| 141303 | 4-t- | 4-t- | 1,3- | H | H |
| | BuPh | BuPh | di-t- | | |
| | | | BuPh | | |
| 141304 | 4-t- | 4-t- | 9-Cbz | H | H |
| | BuPh | BuPh | | | |
| 141305 | 4-t- | 4-t- | t-Bu | t-Bu | t-Bu |
| | BuPh | BuPh | | | |
| 141306 | 4-t- | 4-t- | Ph | t-Bu | t-Bu |
| | BuPh | BuPh | | | |
| 141307 | 4-t- | 4-t- | CN | t-Bu | t-Bu |
| | BuPh | BuPh | | | |
| 141308 | 4-t- | 4-t- | DPA | t-Bu | t-Bu |
| | BuPh | BuPh | | | |
| 141309 | 4-t- | 4-t- | 2- | t-Bu | t-Bu |
| | BuPh | BuPh | DBF | | |
| 141310 | 4-t- | 4-t- | 2- | t-Bu | t-Bu |
| | BuPh | BuPh | DBT | | |
| 141311 | 4-t- | 4-t- | 4-t- | t-Bu | t-Bu |
| | BuPh | BuPh | BuPh | | |
| 141312 | 4-t- | 4-t- | 1,3- | t-Bu | t-Bu |
| | BuPh | BuPh | di-t- | | |
| | | | BuPh | | |
| 141313 | 4-t- | 4-t- | 9-Cbz | t-Bu | t-Bu |
| | BuPh | BuPh | | | |
| 141314 | 4-t- | 4-t- | t-Bu | Ph | Ph |
| | BuPh | BuPh | | | |
| 141315 | 4-t- | 4-t- | Ph | Ph | Ph |
| | BuPh | BuPh | | | |
| 141316 | 4-t- | 4-t- | CN | Ph | Ph |
| | BuPh | BuPh | | | |
| 141317 | 4-t- | 4-t- | DPA | Ph | Ph |
| | BuPh | BuPh | | | |
| 141318 | 4-t- | 4-t- | 2- | Ph | Ph |
| | BuPh | BuPh | DBF | | |
| 141319 | 4-t- | 4-t- | 2- | Ph | Ph |
| | BuPh | BuPh | DBT | | |
| 141320 | 4-t- | 4-t- | 4-t- | Ph | Ph |
| | BuPh | BuPh | BuPh | | |
| 141321 | 4-t- | 4-t- | 1,3- | Ph | Ph |
| | BuPh | BuPh | di-t- | | |
| | | | BuPh | | |
| 141322 | 4-t- | 4-t- | 9-Cbz | Ph | Ph |
| | BuPh | BuPh | | | |
| 141323 | 4-t- | 4-t- | t-Bu | CN | CN |
| | BuPh | BuPh | | | |
| 141324 | 4-t- | 4-t- | Ph | CN | CN |
| | BuPh | BuPh | | | |
| 141325 | 4-t- | 4-t- | CN | CN | CN |
| | BuPh | BuPh | | | |
| 141326 | 4-t- | 4-t- | DPA | CN | CN |
| | BuPh | BuPh | | | |
| 141327 | 4-t- | 4-t- | 2- | CN | CN |
| | BuPh | BuPh | DBF | | |
| 141328 | 4-t- | 4-t- | 2- | CN | CN |
| | BuPh | BuPh | DBT | | |
| 141329 | 4-t- | 4-t- | 4-t- | CN | CN |
| | BuPh | BuPh | BuPh | | |
| 141330 | 4-t- | 4-t- | 1,3- | CN | CN |
| | BuPh | BuPh | di-t- | | |
| | | | BuPh | | |
| 141331 | 4-t- | 4-t- | 9-Cbz | CN | CN |
| | BuPh | BuPh | | | |
| 141332 | 4-t- | 4-t- | t-Bu | 9-Cbz | 9-Cbz |
| | BuPh | BuPh | | | |
| 141333 | 4-t- | 4-t- | Ph | 9-Cbz | 9-Cbz |
| | BuPh | BuPh | | | |
| 141334 | 4-t- | 4-t- | CN | 9-Cbz | 9-Cbz |
| | BuPh | BuPh | | | |
| 141335 | 4-t- | 4-t- | DPA | 9-Cbz | 9-Cbz |
| | BuPh | BuPh | | | |
| 141336 | 4-t- | 4-t- | 2- | 9-Cbz | 9-Cbz |
| | BuPh | BuPh | DBF | | |
| 141337 | 4-t- | 4-t- | 2- | 9-Cbz | 9-Cbz |
| | BuPh | BuPh | DBT | | |
| 141338 | 4-t- | 4-t- | 4-t- | 9-Cbz | 9-Cbz |
| | BuPh | BuPh | BuPh | | |
| 141339 | 4-t- | 4-t- | 1,3- | 9-Cbz | 9-Cbz |
| | BuPh | BuPh | di-t- | | |
| | | | BuPh | | |
| 141340 | 4-t- | 4-t- | 9-Cbz | 9-Cbz | 9-Cbz |
| | BuPh | BuPh | | | |
| 141341 | 4-t- | 4-t- | t-Bu | 4-t- | 4-t- |
| | BuPh | BuPh | | BuPh | BuPh |
| 141342 | 4-t- | 4-t- | Ph | 4-t- | 4-t- |
| | BuPh | BuPh | | BuPh | BuPh |
| 141343 | 4-t- | 4-t- | CN | 4-t- | 4-t- |
| | BuPh | BuPh | | BuPh | BuPh |
| 141344 | 4-t- | 4-t- | DPA | 4-t- | 4-t- |
| | BuPh | BuPh | | BuPh | BuPh |
| 141345 | 4-t- | 4-t- | 2- | 4-t- | 4-t- |
| | BuPh | BuPh | DBF | BuPh | BuPh |
| 141346 | 4-t- | 4-t- | 2- | 4-t- | 4-t- |
| | BuPh | BuPh | DBT | BuPh | BuPh |
| 141347 | 4-t- | 4-t- | 4-t- | 4-t- | 4-t- |
| | BuPh | BuPh | BuPh | BuPh | BuPh |
| 141348 | 4-t- | 4-t- | 1,3- | 4-t- | 4-t- |
| | BuPh | BuPh | di-t- | BuPh | BuPh |
| | | | BuPh | | |
| 141349 | 4-t- | 4-t- | 9-Cbz | 4-t- | 4-t- |
| | BuPh | BuPh | | BuPh | BuPh |
| 141350 | 4-t- | 4-t- | t-Bu | 1,3- | 1,3- |
| | BuPh | BuPh | | di-t- | di-t- |
| | | | | BuPh | BuPh |
| 141351 | 4-t- | 4-t- | Ph | 1,3- | 1,3- |
| | BuPh | BuPh | | di-t- | di-t- |
| | | | | BuPh | BuPh |
| 141352 | 4-t- | 4-t- | CN | 1,3- | 1,3- |
| | BuPh | BuPh | | di-t- | di-t- |
| | | | | BuPh | BuPh |
| 141353 | 4-t- | 4-t- | DPA | 1,3- | 1,3- |
| | BuPh | BuPh | | di-t- | di-t- |
| | | | | BuPh | BuPh |
| 141354 | 4-t- | 4-t- | 2- | 1,3- | 1,3- |
| | BuPh | BuPh | DBF | di-t- | di-t- |
| | | | | BuPh | BuPh |
| 141355 | 4-t- | 4-t- | 2- | 1,3- | 1,3- |
| | BuPh | BuPh | DBT | di-t- | di-t- |
| | | | | BuPh | BuPh |
| 141356 | 4-t- | 4-t- | 4-t- | 1,3- | 1,3- |
| | BuPh | BuPh | BuPh | di-t- | di-t- |
| | | | | BuPh | BuPh |
| 141357 | 4-t- | 4-t- | 1,3- | 1,3- | 1,3- |
| | BuPh | BuPh | di-t- | di-t- | di-t- |
| | | | BuPh | BuPh | BuPh |
| 141358 | 4-t- | 4-t- | 9-Cbz | 1,3- | 1,3- |
| | BuPh | BuPh | | di-t- | di-t- |
| | | | | BuPh | BuPh |
| 141359 | 1,3- | 1,3- | t-Bu | H | H |
| | di-t- | di-t- | | | |
| | BuPh | BuPh | | | |
| 141360 | 1,3- | 1,3- | Ph | H | H |
| | di-t- | di-t- | | | |
| | BuPh | BuPh | | | |
| 141361 | 1,3- | 1,3- | CN | H | H |
| | di-t- | di-t- | | | |
| | BuPh | BuPh | | | |
| 141362 | 1,3- | 1,3- | DPA | H | H |
| | di-t- | di-t- | | | |
| | BuPh | BuPh | | | |
| 141363 | 1,3- | 1,3- | 2- | H | H |
| | di-t- | di-t- | DBF | | |
| | BuPh | BuPh | | | |
| 141364 | 1,3- | 1,3- | 2- | H | H |
| | di-t- | di-t- | DBT | | |
| | BuPh | BuPh | | | |
| 141365 | 1,3- | 1,3- | 4-t- | H | H |
| | di-t- | di-t- | BuPh | | |
| | BuPh | BuPh | | | |
| 141366 | 1,3- | 1,3- | 9-Cbz | H | H |
| | di-t- | di-t- | | | |
| | BuPh | BuPh | | | |
| 141367 | 1,3- | 1,3- | t-Bu | t-Bu | t-Bu |
| | di-t- | di-t- | | | |
| | BuPh | BuPh | | | |
| 141368 | 1,3- | 1,3- | Ph | t-Bu | t-Bu |
| | di-t- | di-t- | | | |
| | BuPh | BuPh | | | |
| 141369 | 1,3- | 1,3- | CN | t-Bu | t-Bu |
| | di-t- | di-t- | | | |
| | BuPh | BuPh | | | |
| 141370 | 1,3- | 1,3- | DPA | t-Bu | t-Bu |
| | di-t- | di-t- | | | |
| | BuPh | BuPh | | | |
| 141371 | 1,3- | 1,3- | 2- | t-Bu | t-Bu |
| | di-t- | di-t- | DBF | | |
| | BuPh | BuPh | | | |
| 141372 | 1,3- | 1,3- | 2- | t-Bu | t-Bu |
| | di-t- | di-t- | DBT | | |
| | BuPh | BuPh | | | |
| 141373 | 1,3- | 1,3- | 4-t- | t-Bu | t-Bu |
| | di-t- | di-t- | BuPh | | |
| | BuPh | BuPh | | | |
| 141374 | 1,3- | 1,3- | 1,3- | t-Bu | t-Bu |
| | di-t- | di-t- | di-t- | | |
| | BuPh | BuPh | BuPh | | |
| 141375 | 1,3- | 1,3- | 9-Cbz | t-Bu | t-Bu |
| | di-t- | di-t- | | | |
| | BuPh | BuPh | | | |
| 141376 | 1,3- | 1,3- | t-Bu | Ph | Ph |
| | di-t- | di-t- | | | |
| | BuPh | BuPh | | | |
| 141377 | 1,3- | 1,3- | Ph | Ph | Ph |
| | di-t- | di-t- | | | |
| | BuPh | BuPh | | | |
| 141378 | 1,3- | 1,3- | CN | Ph | Ph |
| | di-t- | di-t- | | | |
| | BuPh | BuPh | | | |
| 141379 | 1,3- | 1,3- | DPA | Ph | Ph |
| | di-t- | di-t- | | | |
| | BuPh | BuPh | | | |
| 141380 | 1,3- | 1,3- | 2- | Ph | Ph |
| | di-t- | di-t- | DBF | | |
| | BuPh | BuPh | | | |
| 141381 | 1,3- | 1,3- | 2- | Ph | Ph |
| | di-t- | di-t- | DBT | | |
| | BuPh | BuPh | | | |
| 141382 | 1,3- | 1,3- | 4-t- | Ph | Ph |
| | di-t- | di-t- | BuPh | | |
| | BuPh | BuPh | | | |
| 141383 | 1,3- | 1,3- | 1,3- | Ph | Ph |
| | di-t- | di-t- | di-t- | | |
| | BuPh | BuPh | BuPh | | |
| 141384 | 1,3- | 1,3- | 9-Cbz | Ph | Ph |
| | di-t- | di-t- | | | |
| | BuPh | BuPh | | | |
| 141385 | 1,3- | 1,3- | t-Bu | CN | CN |
| | di-t- | di-t- | | | |
| | BuPh | BuPh | | | |
| 141386 | 1,3- | 1,3- | Ph | CN | CN |
| | di-t- | di-t- | | | |
| | BuPh | BuPh | | | |
| 141387 | 1,3- | 1,3- | CN | CN | CN |
| | di-t- | di-t- | | | |
| | BuPh | BuPh | | | |
| 141388 | 1,3- | 1,3- | DPA | CN | CN |
| | di-t- | di-t- | | | |
| | BuPh | BuPh | | | |
| 141389 | 1,3- | 1,3- | 2- | CN | CN |
| | di-t- | di-t- | DBF | | |
| | BuPh | BuPh | | | |
| 141390 | 1,3- | 1,3- | 2- | CN | CN |
| | di-t- | di-t- | DBT | | |
| | BuPh | BuPh | | | |
| 141391 | 1,3- | 1,3- | 4-t- | CN | CN |
| | di-t- | di-t- | BuPh | | |
| | BuPh | BuPh | | | |
| 141392 | 1,3- | 1,3- | 1,3- | CN | CN |
| | di-t- | di-t- | di-t- | | |
| | BuPh | BuPh | BuPh | | |
| 141393 | 1,3- | 1,3- | 9-Cbz | CN | CN |
| | di-t- | di-t- | | | |
| | BuPh | BuPh | | | |
| 141394 | 1,3- | 1,3- | t-Bu | 9-Cbz | 9-Cbz |
| | di-t- | di-t- | | | |
| | BuPh | BuPh | | | |
| 141395 | 1,3- | 1,3- | Ph | 9-Cbz | 9-Cbz |
| | di-t- | di-t- | | | |
| | BuPh | BuPh | | | |
| 141396 | 1,3- | 1,3- | CN | 9-Cbz | 9-Cbz |
| | di-t- | di-t- | | | |
| | BuPh | BuPh | | | |
| 141397 | 1,3- | 1,3- | DPA | 9-Cbz | 9-Cbz |
| | di-t- | di-t- | | | |
| | BuPh | BuPh | | | |
| 141398 | 1,3- | 1,3- | 2- | 9-Cbz | 9-Cbz |
| | di-t- | di-t- | DBF | | |
| | BuPh | BuPh | | | |
| 141399 | 1,3- | 1,3- | 2- | 9-Cbz | 9-Cbz |
| | di-t- | di-t- | DBT | | |
| | BuPh | BuPh | | | |
| 141400 | 1,3- | 1,3- | 4-t- | 9-Cbz | 9-Cbz |
| | di-t- | di-t- | BuPh | | |
| | BuPh | BuPh | | | |
| 141401 | 1,3- | 1,3- | 1,3- | 9-Cbz | 9-Cbz |
| | di-t- | di-t- | di-t- | | |
| | BuPh | BuPh | BuPh | | |
| 141402 | 1,3- | 1,3- | 9-Cbz | 9-Cbz | 9-Cbz |
| | di-t- | di-t- | | | |
| | BuPh | BuPh | | | |
| 141403 | 1,3- | 1,3- | t-Bu | 4-t- | 4-t- |
| | di-t- | di-t- | | BuPh | BuPh |
| | BuPh | BuPh | | | |
| 141404 | 1,3- | 1,3- | Ph | 4-t- | 4-t- |
| | di-t- | di-t- | | BuPh | BuPh |
| | BuPh | BuPh | | | |
| 141405 | 1,3- | 1,3- | CN | 4-t- | 4-t- |
| | di-t- | di-t- | | BuPh | BuPh |
| | BuPh | BuPh | | | |
| 141406 | 1,3- | 1,3- | DPA | 4-t- | 4-t- |
| | di-t- | di-t- | | BuPh | BuPh |
| | BuPh | BuPh | | | |
| 141407 | 1,3- | 1,3- | 2- | 4-t- | 4-t- |
| | di-t- | di-t- | DBF | BuPh | BuPh |
| | BuPh | BuPh | | | |
| 141408 | 1,3- | 1,3- | 2- | 4-t- | 4-t- |
| | di-t- | di-t- | DBT | BuPh | BuPh |
| | BuPh | BuPh | | | |
| 141409 | 1,3- | 1,3- | 4-t- | 4-t- | 4-t- |
| | di-t- | di-t- | BuPh | BuPh | BuPh |
| | BuPh | BuPh | | | |
| 141410 | 1,3- | 1,3- | 1,3- | 4-t- | 4-t- |
| | di-t- | di-t- | di-t- | BuPh | BuPh |
| | BuPh | BuPh | BuPh | | |
| 141411 | 1,3- | 1,3- | 9-Cbz | 4-t- | 4-t- |
| | di-t- | di-t- | | BuPh | BuPh |
| | BuPh | BuPh | | | |
| 141412 | 1,3- | 1,3- | t-Bu | 1,3- | 1,3- |
| | di-t- | di-t- | | di-t- | di-t- |
| | BuPh | BuPh | | BuPh | BuPh |
| 141413 | 1,3- | 1,3- | Ph | 1,3- | 1,3- |
| | di-t- | di-t- | | di-t- | di-t- |
| | BuPh | BuPh | | BuPh | BuPh |
| 141414 | 1,3- | 1,3- | CN | 1,3- | 1,3- |
| | di-t- | di-t- | | di-t- | di-t- |
| | BuPh | BuPh | | BuPh | BuPh |
| 141415 | 1,3- | 1,3- | DPA | 1,3- | 1,3- |
| | di-t- | di-t- | | di-t- | di-t- |
| | BuPh | BuPh | | BuPh | BuPh |
| 141416 | 1,3- | 1,3- | 2- | 1,3- | 1,3- |
| | di-t- | di-t- | DBF | di-t- | di-t- |
| | BuPh | BuPh | | BuPh | BuPh |
| 141417 | 1,3- | 1,3- | 2- | 1,3- | 1,3- |
| | di-t- | di-t- | DBT | di-t- | di-t- |
| | BuPh | BuPh | | BuPh | BuPh |
| 141418 | 1,3- | 1,3- | 4-t- | 1,3- | 1,3- |
| | di-t- | di-t- | BuPh | di-t- | di-t- |
| | BuPh | BuPh | | BuPh | BuPh |
| 141419 | 1,3- | 1,3- | 1,3- | 1,3- | 1,3- |
| | di-t- | di-t- | di-t- | di-t- | di-t- |
| | BuPh | BuPh | BuPh | BuPh | BuPh |
| 141420 | 1,3- | 1,3- | 9-Cbz | 1,3- | 1,3- |
| | di-t- | di-t- | | di-t- | di-t- |
| | BuPh | BuPh | | BuPh | BuPh |

### Group HC7

The heterocyclic compound may be represented by Formula 14-3, and substituents may be defined as shown in Table 7.

TABLE 7

| Compound | | | | | |
|---|---|---|---|---|---|
| No. | R_{11b} | R_{12c} | R_{15C} | R_{18b} | R_{19b} |
| 143001 | t-Bu | t-Bu | t-Bu | t-Bu | t-Bu |
| 143002 | Ph | Ph | t-Bu | Ph | Ph |
| 143003 | t-Bu | t-Bu | H | H | H |
| 143004 | H | H | t-Bu | H | H |
| 143005 | H | H | H | t-Bu | t-Bu |
| 143006 | t-Bu | t-Bu | t-Bu | H | H |
| 143007 | H | H | t-Bu | t-Bu | t-Bu |
| 143008 | t-Bu | t-Bu | H | t-Bu | t-Bu |
| 143009 | Ph | Ph | H | H | H |
| 143010 | H | H | Ph | H | H |
| 143011 | H | H | H | Ph | Ph |
| 143012 | Ph | Ph | Ph | H | H |
| 143013 | H | H | Ph | Ph | Ph |
| 143014 | Ph | Ph | H | Ph | Ph |
| 143015 | CN | CN | H | H | H |
| 143016 | H | H | CN | H | H |
| 143017 | H | H | H | CN | CN |
| 143018 | CN | CN | CN | H | H |
| 143019 | H | H | CN | CN | CN |
| 143020 | CN | CN | H | CN | CN |
| 143021 | 9-Cbz | 9-Cbz | H | H | H |
| 143022 | H | H | 9-Cbz | H | H |
| 143023 | H | H | H | 9-Cbz | 9-Cbz |
| 143024 | 9-Cbz | 9-Cbz | 9-Cbz | H | H |
| 143025 | H | H | 9-Cbz | 9-Cbz | 9-Cbz |
| 143026 | 9-Cbz | 9-Cbz | H | 9-Cbz | 9-Cbz |
| 143027 | Me | Me | H | H | H |
| 143028 | H | H | Me | H | H |
| 143029 | H | H | H | Me | Me |
| 143030 | Me | Me | Me | H | H |
| 143031 | H | H | Me | Me | Me |
| 143032 | Me | Me | H | Me | Me |
| 143033 | CD₃ | CD3 | H | H | H |
| 143034 | H | H | CD3 | H | H |
| 143035 | H | H | H | CD3 | CD3 |
| 143036 | CD3 | CD3 | CD3 | H | H |
| 143037 | H | H | CD3 | CD3 | CD3 |
| 143038 | CD3 | CD3 | H | CD3 | CD3 |
| 143039 | 4-t- | 4-t- | H | H | H |
| | BuPh | BuPh | | | |
| 143040 | H | H | 4-t- | H | H |
| | | | BuPh | | |
| 143041 | H | H | H | 4-t- | 4-t- |
| | | | | BuPh | BuPh |
| 143042 | 4-t- | 4-t- | 4-t- | H | H |
| | BuPh | BuPh | BuPh | | |
| 143043 | H | H | 4-t- | 4-t- | 4-t- |
| | | | BuPh | BuPh | BuPh |
| 143044 | 4-t- | 4-t- | H | 4-t- | 4-t- |
| | BuPh | BuPh | | BuPh | BuPh |
| 143045 | 1,3- | 1,3- | H | H | H |
| | di-t- | di-t- | | | |
| | BuPh | BuPh | | | |
| 143046 | H | H | 1,3- | H | H |
| | | | di-t- | | |
| | | | BuPh | | |
| 143047 | H | H | H | 1,3- | 1,3- |
| | | | | di-t- | di-t- |
| | | | | BuPh | BuPh |
| 143048 | 1,3- | 1,3- | 1,3- | H | H |
| | di-t- | di-t- | di-t- | | |
| | BuPh | BuPh | BuPh | | |
| 143049 | H | H | 1,3- | 1,3- | 1,3- |
| | | | di-t- | di-t- | di-t- |
| | | | BuPh | BuPh | BuPh |
| 143050 | 1,3- | 1,3- | H | 1,3- | 1,3- |
| | di-t- | di-t- | | di-t- | di-t- |
| | BuPh | BuPh | | BuPh | BuPh |
| 143051 | t-Bu | t-Bu | Ph | H | H |
| 143052 | t-Bu | t-Bu | CN | H | H |
| 143053 | t-Bu | t-Bu | DPA | H | H |
| 143054 | t-Bu | t-Bu | 2- | H | H |
| | | | DBF | | |
| 143055 | t-Bu | t-Bu | 2- | H | H |
| | | | DBT | | |
| 143056 | t-Bu | t-Bu | 4-t- | H | H |
| | | | BuPh | | |
| 143057 | t-Bu | t-Bu | 1,3- | H | H |
| | | | di-t- | | |
| | | | BuPh | | |
| 143058 | t-Bu | t-Bu | 9-Cbz | H | H |
| 143059 | t-Bu | t-Bu | Ph | t-Bu | t-Bu |
| 143060 | t-Bu | t-Bu | CN | t-Bu | t-Bu |
| 143061 | t-Bu | t-Bu | DPA | t-Bu | t-Bu |
| 143062 | t-Bu | t-Bu | 2- | t-Bu | t-Bu |
| | | | DBF | | |
| 143063 | t-Bu | t-Bu | 2- | t-Bu | t-Bu |
| | | | DBT | | |
| 143064 | t-Bu | t-Bu | 4-t- | t-Bu | t-Bu |
| | | | BuPh | | |
| 143065 | t-Bu | t-Bu | 1,3- | t-Bu | t-Bu |
| | | | di-t- | | |
| | | | BuPh | | |
| 143066 | t-Bu | t-Bu | 9-Cbz | t-Bu | t-Bu |
| 143067 | t-Bu | t-Bu | t-Bu | Ph | Ph |
| 143068 | t-Bu | t-Bu | Ph | Ph | Ph |
| 143069 | t-Bu | t-Bu | CN | Ph | Ph |
| 143070 | t-Bu | t-Bu | DPA | Ph | Ph |
| 143071 | t-Bu | t-Bu | 2- | Ph | Ph |
| | | | DBF | | |
| 143072 | t-Bu | t-Bu | 2- | Ph | Ph |
| | | | DBT | | |
| 143073 | t-Bu | t-Bu | 4-t- | Ph | Ph |
| | | | BuPh | | |
| 143074 | t-Bu | t-Bu | 1,3- | Ph | Ph |
| | | | di-t- | | |
| | | | BuPh | | |
| 143075 | t-Bu | t-Bu | 9-Cbz | Ph | Ph |
| 143076 | t-Bu | t-Bu | t-Bu | CN | CN |
| 143077 | t-Bu | t-Bu | Ph | CN | CN |
| 143078 | t-Bu | t-Bu | CN | CN | CN |
| 143079 | t-Bu | t-Bu | DPA | CN | CN |
| 143080 | t-Bu | t-Bu | 2- | CN | CN |
| | | | DBF | | |
| 143081 | t-Bu | t-Bu | 2- | CN | CN |
| | | | DBT | | |
| 143082 | t-Bu | t-Bu | 4-t- | CN | CN |
| | | | BuPh | | |
| 143083 | t-Bu | t-Bu | 1,3- | CN | CN |
| | | | di-t- | | |
| | | | BuPh | | |
| 143084 | t-Bu | t-Bu | 9-Cbz | CN | CN |
| 143085 | t-Bu | t-Bu | t-Bu | 9-Cbz | 9-Cbz |
| 143086 | t-Bu | t-Bu | Ph | 9-Cbz | 9-Cbz |
| 143087 | t-Bu | t-Bu | CN | 9-Cbz | 9-Cbz |
| 143088 | t-Bu | t-Bu | DPA | 9-Cbz | 9-Cbz |
| 143089 | t-Bu | t-Bu | 2- | 9-Cbz | 9-Cbz |
| | | | DBF | | |
| 143090 | t-Bu | t-Bu | 2- | 9-Cbz | 9-Cbz |
| | | | DBT | | |
| 143091 | t-Bu | t-Bu | 4-t- | 9-Cbz | 9-Cbz |
| | | | BuPh | | |
| 143092 | t-Bu | t-Bu | 1,3- | 9-Cbz | 9-Cbz |
| | | | di-t- | | |
| | | | BuPh | | |
| 143093 | t-Bu | t-Bu | 9-Cbz | 9-Cbz | 9-Cbz |
| 143094 | t-Bu | t-Bu | t-Bu | 4-t- | 4-t- |
| | | | | BuPh | BuPh |
| 143095 | t-Bu | t-Bu | Ph | 4-t- | 4-t- |
| | | | | BuPh | BuPh |
| 143096 | t-Bu | t-Bu | CN | 4-t- | 4-t- |
| | | | | BuPh | BuPh |
| 143097 | t-Bu | t-Bu | DPA | 4-t- | 4-t- |
| | | | | BuPh | BuPh |
| 143098 | t-Bu | t-Bu | 2- | 4-t- | 4-t- |
| | | | DBF | BuPh | BuPh |
| 143099 | t-Bu | t-Bu | 2- | 4-t- | 4-t- |
| | | | DBT | BuPh | BuPh |
| 143100 | t-Bu | t-Bu | 4-t- | 4-t- | 4-t- |
| | | | BuPh | BuPh | BuPh |
| 143101 | t-Bu | t-Bu | 1,3- | 4-t- | 4-t- |
| | | | di-t- | BuPh | BuPh |
| | | | BuPh | | |
| 143102 | t-Bu | t-Bu | 9-Cbz | 4-t- | 4-t- |
| | | | | BuPh | BuPh |
| 143103 | t-Bu | t-Bu | t-Bu | 1,3- | 1,3- |
| | | | | di-t- | di-t- |
| | | | | BuPh | BuPh |
| 143104 | t-Bu | t-Bu | Ph | 1,3- | 1,3- |
| | | | | di-t- | di-t- |
| | | | | BuPh | BuPh |
| 143105 | t-Bu | t-Bu | CN | 1,3- | 1,3- |
| | | | | di-t- | di-t- |
| | | | | BuPh | BuPh |
| 143106 | t-Bu | t-Bu | DPA | 1,3- | 1,3- |
| | | | | di-t- | di-t- |
| | | | | BuPh | BuPh |
| 143107 | t-Bu | t-Bu | 2- | 1,3- | 1,3- |
| | | | DBF | di-t- | di-t- |
| | | | | BuPh | BuPh |
| 143108 | t-Bu | t-Bu | 2- | 1,3- | 1,3- |
| | | | DBT | di-t- | di-t- |
| | | | | BuPh | BuPh |
| 143109 | t-Bu | t-Bu | 4-t- | 1,3- | 1,3- |
| | | | BuPh | di-t- | di-t- |
| | | | | BuPh | BuPh |
| 143110 | t-Bu | t-Bu | 1,3- | 1,3- | 1,3- |
| | | | di-t- | di-t- | di-t- |
| | | | BuPh | BuPh | BuPh |
| 143111 | t-Bu | t-Bu | 9-Cbz | 1,3- | 1,3- |
| | | | | di-t- | di-t- |
| | | | | BuPh | BuPh |
| 143112 | Ph | Ph | t-Bu | H | H |
| 143113 | Ph | Ph | CN | H | H |
| 143114 | Ph | Ph | DPA | H | H |
| 143115 | Ph | Ph | 2- | H | H |
| | | | DBF | | |
| 143116 | Ph | Ph | 2- | H | H |
| | | | DBT | | |
| 143117 | Ph | Ph | 4-t- | H | H |
| | | | BuPh | | |
| 143118 | Ph | Ph | 1,3- | H | H |
| | | | di-t- | | |
| | | | BuPh | | |
| 143119 | Ph | Ph | 9-Cbz | H | H |
| 143120 | Ph | Ph | t-Bu | t-Bu | t-Bu |
| 143121 | Ph | Ph | Ph | t-Bu | t-Bu |
| 143122 | Ph | Ph | CN | t-Bu | t-Bu |
| 143123 | Ph | Ph | DPA | t-Bu | t-Bu |
| 143124 | Ph | Ph | 2- | t-Bu | t-Bu |
| | | | DBF | | |
| 143125 | Ph | Ph | 2- | t-Bu | t-Bu |
| | | | DBT | | |
| 143126 | Ph | Ph | 4-t- | t-Bu | t-Bu |
| | | | BuPh | | |
| 143127 | Ph | Ph | 1,3- | t-Bu | t-Bu |
| | | | di-t- | | |
| | | | BuPh | | |
| 143128 | Ph | Ph | 9-Cbz | t-Bu | t-Bu |
| 143129 | Ph | Ph | Ph | Ph | Ph |
| 143130 | Ph | Ph | CN | Ph | Ph |
| 143131 | Ph | Ph | DPA | Ph | Ph |
| 143132 | Ph | Ph | 2- | Ph | Ph |
| | | | DBF | | |
| 143133 | Ph | Ph | 2- | Ph | Ph |
| | | | DBT | | |
| 143134 | Ph | Ph | 4-t- | Ph | Ph |
| | | | BuPh | | |
| 143135 | Ph | Ph | 1,3- | Ph | Ph |
| | | | di-t- | | |
| | | | BuPh | | |
| 143136 | Ph | Ph | 9-Cbz | Ph | Ph |
| 143137 | Ph | Ph | t-Bu | CN | CN |
| 143138 | Ph | Ph | Ph | CN | CN |
| 143139 | Ph | Ph | CN | CN | CN |
| 143140 | Ph | Ph | DPA | CN | CN |
| 143141 | Ph | Ph | 2- | CN | CN |
| | | | DBF | | |
| 143142 | Ph | Ph | 2- | CN | CN |
| | | | DBT | | |
| 143143 | Ph | Ph | 4-t- | CN | CN |
| | | | BuPh | | |
| 143144 | Ph | Ph | 1,3- | CN | CN |
| | | | di-t- | | |
| | | | BuPh | | |
| 143145 | Ph | Ph | 9-Cbz | CN | CN |
| 143146 | Ph | Ph | t-Bu | 9-Cbz | 9-Cbz |
| 143147 | Ph | Ph | Ph | 9-Cbz | 9-Cbz |
| 143148 | Ph | Ph | CN | 9-Cbz | 9-Cbz |
| 143149 | Ph | Ph | DPA | 9-Cbz | 9-Cbz |
| 143150 | Ph | Ph | 2- | 9-Cbz | 9-Cbz |
| | | | DBF | | |
| 143151 | Ph | Ph | 2- | 9-Cbz | 9-Cbz |
| | | | DBT | | |
| 143152 | Ph | Ph | 4-t- | 9-Cbz | 9-Cbz |
| | | | BuPh | | |
| 143153 | Ph | Ph | 1,3- | 9-Cbz | 9-Cbz |
| | | | di-t- | | |
| | | | BuPh | | |
| 143154 | Ph | Ph | 9-Cbz | 9-Cbz | 9-Cbz |
| 143155 | Ph | Ph | t-Bu | 4-t- | 4-t- |
| | | | | BuPh | BuPh |
| 143156 | Ph | Ph | Ph | 4-t- | 4-t- |
| | | | | BuPh | BuPh |
| 143157 | Ph | Ph | CN | 4-t- | 4-t- |
| | | | | BuPh | BuPh |
| 143158 | Ph | Ph | DPA | 4-t- | 4-t- |
| | | | | BuPh | BuPh |
| 143159 | Ph | Ph | 2- | 4-t- | 4-t- |
| | | | DBF | BuPh | BuPh |
| 143160 | Ph | Ph | 2- | 4-t- | 4-t- |
| | | | DBT | BuPh | BuPh |
| 143161 | Ph | Ph | 4-t- | 4-t- | 4-t- |
| | | | BuPh | BuPh | BuPh |
| 143162 | Ph | Ph | 1,3- | 4-t- | 4-t- |
| | | | di-t- | BuPh | BuPh |
| | | | BuPh | | |
| 143163 | Ph | Ph | 9-Cbz | 4-t- | 4-t- |
| | | | | BuPh | BuPh |
| 143164 | Ph | Ph | t-Bu | 1,3- | 1,3- |
| | | | | di-t- | di-t- |
| | | | | BuPh | BuPh |
| 143165 | Ph | Ph | Ph | 1,3- | 1,3- |
| | | | | di-t- | di-t- |
| | | | | BuPh | BuPh |
| 143166 | Ph | Ph | CN | 1,3- | 1,3- |
| | | | | di-t- | di-t- |
| | | | | BuPh | BuPh |
| 143167 | Ph | Ph | DPA | 1,3- | 1,3- |
| | | | | di-t- | di-t- |
| | | | | BuPh | BuPh |
| 143168 | Ph | Ph | 2- | 1,3- | 1,3- |
| | | | DBF | di-t- | di-t- |
| | | | | BuPh | BuPh |
| 143169 | Ph | Ph | 2- | 1,3- | 1,3- |
| | | | DBT | di-t- | di-t- |
| | | | | BuPh | BuPh |
| 143170 | Ph | Ph | 4-t- | 1,3- | 1,3- |
| | | | BuPh | di-t- | di-t- |
| | | | | BuPh | BuPh |
| 143171 | Ph | Ph | 1,3- | 1,3- | 1,3- |
| | | | di-t- | di-t- | di-t- |
| | | | BuPh | BuPh | BuPh |
| 143172 | Ph | Ph | 9-Cbz | 1,3- | 1,3- |
| | | | | di-t- | di-t- |
| | | | | BuPh | BuPh |
| 143173 | 9-Cbz | 9-Cbz | t-Bu | H | H |
| 143174 | 9-Cbz | 9-Cbz | Ph | H | H |
| 143175 | 9-Cbz | 9-Cbz | CN | H | H |
| 143176 | 9-Cbz | 9-Cbz | DPA | H | H |
| 143177 | 9-Cbz | 9-Cbz | 2- | H | H |
| | | | DBF | | |
| 143178 | 9-Cbz | 9-Cbz | 2- | H | H |
| | | | DBT | | |
| 143179 | 9-Cbz | 9-Cbz | 4-t- | H | H |
| | | | BuPh | | |
| 143180 | 9-Cbz | 9-Cbz | 1,3- | H | H |
| | | | di-t- | | |
| | | | BuPh | | |
| 143181 | 9-Cbz | 9-Cbz | t-Bu | t-Bu | t-Bu |
| 143182 | 9-Cbz | 9-Cbz | Ph | t-Bu | t-Bu |
| 143183 | 9-Cbz | 9-Cbz | CN | t-Bu | t-Bu |
| 143184 | 9-Cbz | 9-Cbz | DPA | t-Bu | t-Bu |
| 143185 | 9-Cbz | 9-Cbz | 2- | t-Bu | t-Bu |
| | | | DBF | | |
| 143186 | 9-Cbz | 9-Cbz | 2- | t-Bu | t-Bu |
| | | | DBT | | |
| 143187 | 9-Cbz | 9-Cbz | 4-t- | t-Bu | t-Bu |
| | | | BuPh | | |
| 143188 | 9-Cbz | 9-Cbz | 1,3- | t-Bu | t-Bu |
| | | | di-t- | | |
| | | | BuPh | | |
| 143189 | 9-Cbz | 9-Cbz | 9-Cbz | t-Bu | t-Bu |
| 143190 | 9-Cbz | 9-Cbz | t-Bu | Ph | Ph |
| 143191 | 9-Cbz | 9-Cbz | Ph | Ph | Ph |
| 143192 | 9-Cbz | 9-Cbz | CN | Ph | Ph |
| 143193 | 9-Cbz | 9-Cbz | DPA | Ph | Ph |
| 143194 | 9-Cbz | 9-Cbz | 2- | Ph | Ph |
| | | | DBF | | |
| 143195 | 9-Cbz | 9-Cbz | 2- | Ph | Ph |
| | | | DBT | | |
| 143196 | 9-Cbz | 9-Cbz | 4-t- | Ph | Ph |
| | | | BuPh | | |
| 143197 | 9-Cbz | 9-Cbz | 1,3- | Ph | Ph |
| | | | di-t- | | |
| | | | BuPh | | |
| 143198 | 9-Cbz | 9-Cbz | 9-Cbz | Ph | Ph |
| 143199 | 9-Cbz | 9-Cbz | t-Bu | CN | CN |
| 143200 | 9-Cbz | 9-Cbz | Ph | CN | CN |
| 143201 | 9-Cbz | 9-Cbz | CN | CN | CN |
| 143202 | 9-Cbz | 9-Cbz | DPA | CN | CN |
| 143203 | 9-Cbz | 9-Cbz | 2- | CN | CN |
| | | | DBF | | |
| 143204 | 9-Cbz | 9-Cbz | 2- | CN | CN |
| | | | DBT | | |
| 143205 | 9-Cbz | 9-Cbz | 4-t- | CN | CN |
| | | | BuPh | | |
| 143206 | 9-Cbz | 9-Cbz | 1,3- | CN | CN |
| | | | di-t- | | |
| | | | BuPh | | |
| 143207 | 9-Cbz | 9-Cbz | 9-Cbz | CN | CN |
| 143208 | 9-Cbz | 9-Cbz | t-Bu | 9-Cbz | 9-Cbz |
| 143209 | 9-Cbz | 9-Cbz | Ph | 9-Cbz | 9-Cbz |
| 143210 | 9-Cbz | 9-Cbz | CN | 9-Cbz | 9-Cbz |
| 143211 | 9-Cbz | 9-Cbz | DPA | 9-Cbz | 9-Cbz |
| 143212 | 9-Cbz | 9-Cbz | 2- | 9-Cbz | 9-Cbz |
| | | | DBF | | |
| 143213 | 9-Cbz | 9-Cbz | 2- | 9-Cbz | 9-Cbz |
| | | | DBT | | |
| 143214 | 9-Cbz | 9-Cbz | 4-t- | 9-Cbz | 9-Cbz |
| | | | BuPh | | |
| 143215 | 9-Cbz | 9-Cbz | 1,3- | 9-Cbz | 9-Cbz |
| | | | di-t- | | |
| | | | BuPh | | |
| 143216 | 9-Cbz | 9-Cbz | 9-Cbz | 9-Cbz | 9-Cbz |
| 143217 | 9-Cbz | 9-Cbz | t-Bu | 4-t- | 4-t- |
| | | | | BuPh | BuPh |
| 143218 | 9-Cbz | 9-Cbz | Ph | 4-t- | 4-t- |
| | | | | BuPh | BuPh |
| 143219 | 9-Cbz | 9-Cbz | CN | 4-t- | 4-t- |
| | | | | BuPh | BuPh |
| 143220 | 9-Cbz | 9-Cbz | DPA | 4-t- | 4-t- |
| | | | | BuPh | BuPh |
| 143221 | 9-Cbz | 9-Cbz | 2- | 4-t- | 4-t- |
| | | | DBF | BuPh | BuPh |
| 143222 | 9-Cbz | 9-Cbz | 2- | 4-t- | 4-t- |
| | | | DBT | BuPh | BuPh |
| 143223 | 9-Cbz | 9-Cbz | 4-t- | 4-t- | 4-t- |
| | | | BuPh | BuPh | BuPh |
| 143224 | 9-Cbz | 9-Cbz | 1,3- | 4-t- | 4-t- |
| | | | di-t- | BuPh | BuPh |
| | | | BuPh | | |
| 143225 | 9-Cbz | 9-Cbz | 9-Cbz | 4-t- | 4-t- |
| | | | | BuPh | BuPh |
| 143226 | 9-Cbz | 9-Cbz | t-Bu | 1,3- | 1,3- |
| | | | | di-t- | di-t- |
| | | | | BuPh | BuPh |
| 143227 | 9-Cbz | 9-Cbz | Ph | 1,3- | 1,3- |
| | | | | di-t- | di-t- |
| | | | | BuPh | BuPh |
| 143228 | 9-Cbz | 9-Cbz | CN | 1,3- | 1,3- |
| | | | | di-t- | di-t- |
| | | | | BuPh | BuPh |
| 143229 | 9-Cbz | 9-Cbz | DPA | 1,3- | 1,3- |
| | | | | di-t- | di-t- |
| | | | | BuPh | BuPh |
| 143230 | 9-Cbz | 9-Cbz | 2- | 1,3- | 1,3- |
| | | | DBF | di-t- | di-t- |
| | | | | BuPh | BuPh |
| 143231 | 9-Cbz | 9-Cbz | 2- | 1,3- | 1,3- |
| | | | DBT | di-t- | di-t- |
| | | | | BuPh | BuPh |
| 143232 | 9-Cbz | 9-Cbz | 4-t- | 1,3- | 1,3- |
| | | | BuPh | di-t- | di-t- |
| | | | | BuPh | BuPh |
| 143233 | 9-Cbz | 9-Cbz | 1,3- | 1,3- | 1,3- |
| | | | di-t- | di-t- | di-t- |
| | | | BuPh | BuPh | BuPh |
| 143234 | 9-Cbz | 9-Cbz | 9-Cbz | 1,3- | 1,3- |
| | | | | di-t- | di-t- |
| | | | | BuPh | BuPh |
| 143235 | CN | CN | t-Bu | H | H |
| 143236 | CN | CN | Ph | H | H |
| 143237 | CN | CN | DPA | H | H |
| 143238 | CN | CN | 2- | H | H |
| | | | DBF | | |
| 143239 | CN | CN | 2- | H | H |
| | | | DBT | | |
| 143240 | CN | CN | 4-t- | H | H |
| | | | BuPh | | |
| 143241 | CN | CN | 1,3- | H | H |
| | | | di-t- | | |
| | | | BuPh | | |
| 143242 | CN | CN | 9-Cbz | H | H |
| 143243 | CN | CN | t-Bu | t-Bu | t-Bu |
| 143244 | CN | CN | Ph | t-Bu | t-Bu |
| 143245 | CN | CN | CN | t-Bu | t-Bu |
| 143246 | CN | CN | DPA | t-Bu | t-Bu |
| 143247 | CN | CN | 2- | t-Bu | t-Bu |
| | | | DBF | | |
| 143248 | CN | CN | 2- | t-Bu | t-Bu |
| | | | DBT | | |
| 143249 | CN | CN | 4-t- | t-Bu | t-Bu |
| | | | BuPh | | |
| 143250 | CN | CN | 1,3- | t-Bu | t-Bu |
| | | | di-t- | | |
| | | | BuPh | | |
| 143251 | CN | CN | 9-Cbz | t-Bu | t-Bu |
| 143252 | CN | CN | t-Bu | Ph | Ph |
| 143253 | CN | CN | Ph | Ph | Ph |
| 143254 | CN | CN | CN | Ph | Ph |
| 143255 | CN | CN | DPA | Ph | Ph |
| 143256 | CN | CN | 2- | Ph | Ph |
| | | | DBF | | |
| 143257 | CN | CN | 2- | Ph | Ph |
| | | | DBT | | |
| 143258 | CN | CN | 4-t- | Ph | Ph |
| | | | BuPh | | |
| 143259 | CN | CN | 1,3- | Ph | Ph |
| | | | di-t- | | |
| | | | BuPh | | |
| 143260 | CN | CN | 9-Cbz | Ph | Ph |
| 143261 | CN | CN | t-Bu | CN | CN |
| 143262 | CN | CN | Ph | CN | CN |
| 143263 | CN | CN | CN | CN | CN |
| 143264 | CN | CN | DPA | CN | CN |
| 143265 | CN | CN | 2- | CN | CN |
| | | | DBF | | |
| 143266 | CN | CN | 2- | CN | CN |
| | | | DBT | | |
| 143267 | CN | CN | 4-t- | CN | CN |
| | | | BuPh | | |
| 143268 | CN | CN | 1,3- | CN | CN |
| | | | di-t- | | |
| | | | BuPh | | |
| 143269 | CN | CN | 9-Cbz | CN | CN |
| 143270 | CN | CN | t-Bu | 9-Cbz | 9-Cbz |
| 143271 | CN | CN | Ph | 9-Cbz | 9-Cbz |
| 143272 | CN | CN | CN | 9-Cbz | 9-Cbz |
| 143273 | CN | CN | DPA | 9-Cbz | 9-Cbz |
| 143274 | CN | CN | 2- | 9-Cbz | 9-Cbz |
| | | | DBF | | |
| 143275 | CN | CN | 2- | 9-Cbz | 9-Cbz |
| | | | DBT | | |
| 143276 | CN | CN | 4-t- | 9-Cbz | 9-Cbz |
| | | | BuPh | | |
| 143277 | CN | CN | 1,3- | 9-Cbz | 9-Cbz |
| | | | di-t- | | |
| | | | BuPh | | |
| 143278 | CN | CN | 9-Cbz | 9-Cbz | 9-Cbz |
| 143279 | CN | CN | t-Bu | 4-t- | 4-t- |
| | | | | BuPh | BuPh |
| 143280 | CN | CN | Ph | 4-t- | 4-t- |
| | | | | BuPh | BuPh |
| 143281 | CN | CN | CN | 4-t- | 4-t- |
| | | | | BuPh | BuPh |
| 143282 | CN | CN | DPA | 4-t- | 4-t- |
| | | | | BuPh | BuPh |
| 143283 | CN | CN | 2- | 4-t- | 4-t- |
| | | | DBF | BuPh | BuPh |
| 143284 | CN | CN | 2- | 4-t- | 4-t- |
| | | | DBT | BuPh | BuPh |
| 143285 | CN | CN | 4-t- | 4-t- | 4-t- |
| | | | BuPh | BuPh | BuPh |
| 143286 | CN | CN | 1,3- | 4-t- | 4-t- |
| | | | di-t- | BuPh | BuPh |
| | | | BuPh | | |
| 143287 | CN | CN | 9-Cbz | 4-t- | 4-t- |
| | | | | BuPh | BuPh |
| 143288 | CN | CN | t-Bu | 1,3- | 1,3- |
| | | | | di-t- | di-t- |
| | | | | BuPh | BuPh |
| 143289 | CN | CN | Ph | 1,3- | 1,3- |
| | | | | di-t- | di-t- |
| | | | | BuPh | BuPh |
| 143290 | CN | CN | CN | 1,3- | 1,3- |
| | | | | di-t- | di-t- |
| | | | | BuPh | BuPh |
| 143291 | CN | CN | DPA | 1,3- | 1,3- |
| | | | | di-t- | di-t- |
| | | | | BuPh | BuPh |
| 143292 | CN | CN | 2- | 1,3- | 1,3- |
| | | | DBF | di-t- | di-t- |
| | | | | BuPh | BuPh |
| 143293 | CN | CN | 2- | 1,3- | 1,3- |
| | | | DBT | di-t- | di-t- |
| | | | | BuPh | BuPh |
| 143294 | CN | CN | 4-t- | 1,3- | 1,3- |
| | | | BuPh | di-t- | di-t- |
| | | | | BuPh | BuPh |
| 143295 | CN | CN | 1,3- | 1,3- | 1,3- |
| | | | di-t- | di-t- | di-t- |
| | | | BuPh | BuPh | BuPh |
| 143296 | CN | CN | 9-Cbz | 1,3- | 1,3- |
| | | | | di-t- | di-t- |
| | | | | BuPh | BuPh |
| 143297 | 4-t- | 4-t- | t-Bu | H | H |
| | BuPh | BuPh | | | |
| 143298 | 4-t- | 4-t- | Ph | H | H |
| | BuPh | BuPh | | | |
| 143299 | 4-t- | 4-t- | CN | H | H |
| | BuPh | BuPh | | | |
| 143300 | 4-t- | 4-t- | DPA | H | H |
| | BuPh | BuPh | | | |
| 143301 | 4-t- | 4-t- | 2- | H | H |
| | BuPh | BuPh | DBF | | |
| 143302 | 4-t- | 4-t- | 2- | H | H |
| | BuPh | BuPh | DBT | | |
| 143303 | 4-t- | 4-t- | 1,3- | H | H |
| | BuPh | BuPh | di-t- | | |
| | | | BuPh | | |
| 143304 | 4-t- | 4-t- | 9-Cbz | H | H |
| | BuPh | BuPh | | | |
| 143305 | 4-t- | 4-t- | t-Bu | t-Bu | t-Bu |
| | BuPh | BuPh | | | |
| 143306 | 4-t- | 4-t- | Ph | t-Bu | t-Bu |
| | BuPh | BuPh | | | |
| 143307 | 4-t- | 4-t- | CN | t-Bu | t-Bu |
| | BuPh | BuPh | | | |
| 143308 | 4-t- | 4-t- | DPA | t-Bu | t-Bu |
| | BuPh | BuPh | | | |
| 143309 | 4-t- | 4-t- | 2- | t-Bu | t-Bu |
| | BuPh | BuPh | DBF | | |
| 143310 | 4-t- | 4-t- | 2- | t-Bu | t-Bu |
| | BuPh | BuPh | DBT | | |
| 143311 | 4-t- | 4-t- | 4-t- | t-Bu | t-Bu |
| | BuPh | BuPh | BuPh | | |
| 143312 | 4-t- | 4-t- | 1,3- | t-Bu | t-Bu |
| | BuPh | BuPh | di-t- | | |
| | | | BuPh | | |
| 143313 | 4-t- | 4-t- | 9-Cbz | t-Bu | t-Bu |
| | BuPh | BuPh | | | |
| 143314 | 4-t- | 4-t- | t-Bu | Ph | Ph |
| | BuPh | BuPh | | | |
| 143315 | 4-t- | 4-t- | Ph | Ph | Ph |
| | BuPh | BuPh | | | |
| 143316 | 4-t- | 4-t- | CN | Ph | Ph |
| | BuPh | BuPh | | | |
| 143317 | 4-t- | 4-t- | DPA | Ph | Ph |
| | BuPh | BuPh | | | |
| 143318 | 4-t- | 4-t- | 2- | Ph | Ph |
| | BuPh | BuPh | DBF | | |
| 143319 | 4-t- | 4-t- | 2- | Ph | Ph |
| | BuPh | BuPh | DBT | | |
| 143320 | 4-t- | 4-t- | 4-t- | Ph | Ph |
| | BuPh | BuPh | BuPh | | |
| 143321 | 4-t- | 4-t- | 1,3- | Ph | Ph |
| | BuPh | BuPh | di-t- | | |
| | | | BuPh | | |
| 143322 | 4-t- | 4-t- | 9-Cbz | Ph | Ph |
| | BuPh | BuPh | | | |
| 143323 | 4-t- | 4-t- | t-Bu | CN | CN |
| | BuPh | BuPh | | | |
| 143324 | 4-t- | 4-t- | Ph | CN | CN |
| | BuPh | BuPh | | | |
| 143325 | 4-t- | 4-t- | CN | CN | CN |
| | BuPh | BuPh | | | |
| 143326 | 4-t- | 4-t- | DPA | CN | CN |
| | BuPh | BuPh | | | |
| 143327 | 4-t- | 4-t- | 2- | CN | CN |
| | BuPh | BuPh | DBF | | |
| 143328 | 4-t- | 4-t- | 2- | CN | CN |
| | BuPh | BuPh | DBT | | |
| 143329 | 4-t- | 4-t- | 4-t- | CN | CN |
| | BuPh | BuPh | BuPh | | |
| 143330 | 4-t- | 4-t- | 1,3- | CN | CN |
| | BuPh | BuPh | di-t- | | |
| | | | BuPh | | |
| 143331 | 4-t- | 4-t- | 9-Cbz | CN | CN |
| | BuPh | BuPh | | | |
| 143332 | 4-t- | 4-t- | t-Bu | 9-Cbz | 9-Cbz |
| | BuPh | BuPh | | | |
| 143333 | 4-t- | 4-t- | Ph | 9-Cbz | 9-Cbz |
| | BuPh | BuPh | | | |
| 143334 | 4-t- | 4-t- | CN | 9-Cbz | 9-Cbz |
| | BuPh | BuPh | | | |
| 143335 | 4-t- | 4-t- | DPA | 9-Cbz | 9-Cbz |
| | BuPh | BuPh | | | |
| 143336 | 4-t- | 4-t- | 2- | 9-Cbz | 9-Cbz |
| | BuPh | BuPh | DBF | | |
| 143337 | 4-t- | 4-t- | 2- | 9-Cbz | 9-Cbz |
| | BuPh | BuPh | DBT | | |
| 143338 | 4-t- | 4-t- | 4-t- | 9-Cbz | 9-Cbz |
| | BuPh | BuPh | BuPh | | |
| 143339 | 4-t- | 4-t- | 1,3- | 9-Cbz | 9-Cbz |
| | BuPh | BuPh | di-t- | | |
| | | | BuPh | | |
| 143340 | 4-t- | 4-t- | 9-Cbz | 9-Cbz | 9-Cbz |
| | BuPh | BuPh | | | |
| 143341 | 4-t- | 4-t- | t-Bu | 4-t- | 4-t- |
| | BuPh | BuPh | | BuPh | BuPh |
| 143342 | 4-t- | 4-t- | Ph | 4-t- | 4-t- |
| | BuPh | BuPh | | BuPh | BuPh |
| 143343 | 4-t- | 4-t- | CN | 4-t- | 4-t- |
| | BuPh | BuPh | | BuPh | BuPh |
| 143344 | 4-t- | 4-t- | DPA | 4-t- | 4-t- |
| | BuPh | BuPh | | BuPh | BuPh |
| 143345 | 4-t- | 4-t- | 2- | 4-t- | 4-t- |
| | BuPh | BuPh | DBF | BuPh | BuPh |
| 143346 | 4-t- | 4-t- | 2- | 4-t- | 4-t- |
| | BuPh | BuPh | DBT | BuPh | BuPh |
| 143347 | 4-t- | 4-t- | 4-t- | 4-t- | 4-t- |
| | BuPh | BuPh | BuPh | BuPh | BuPh |
| 143348 | 4-t- | 4-t- | 1,3- | 4-t- | 4-t- |
| | BuPh | BuPh | di-t- | BuPh | BuPh |
| | | | BuPh | | |
| 143349 | 4-t- | 4-t- | 9-Cbz | 4-t- | 4-t- |
| | BuPh | BuPh | | BuPh | BuPh |
| 143350 | 4-t- | 4-t- | t-Bu | 1,3- | 1,3- |
| | BuPh | BuPh | | di-t- | di-t- |
| | | | | BuPh | BuPh |
| 143351 | 4-t- | 4-t- | Ph | 1,3- | 1,3- |
| | BuPh | BuPh | | di-t- | di-t- |
| | | | | BuPh | BuPh |
| 143352 | 4-t- | 4-t- | CN | 1,3- | 1,3- |
| | BuPh | BuPh | | di-t- | di-t- |
| | | | | BuPh | BuPh |
| 143353 | 4-t- | 4-t- | DPA | 1,3- | 1,3- |
| | BuPh | BuPh | | di-t- | di-t- |
| | | | | BuPh | BuPh |
| 143354 | 4-t- | 4-t- | 2- | 1,3- | 1,3- |
| | BuPh | BuPh | DBF | di-t- | di-t- |
| | | | | BuPh | BuPh |
| 143355 | 4-t- | 4-t- | 2- | 1,3- | 1,3- |
| | BuPh | BuPh | DBT | di-t- | di-t- |
| | | | | BuPh | BuPh |
| 143356 | 4-t- | 4-t- | 4-t- | 1,3- | 1,3- |
| | BuPh | BuPh | BuPh | di-t- | di-t- |
| | | | | BuPh | BuPh |
| 143357 | 4-t- | 4-t- | 1,3- | 1,3- | 1,3- |
| | BuPh | BuPh | di-t- | di-t- | di-t- |
| | | | BuPh | BuPh | BuPh |
| 143358 | 4-t- | 4-t- | 9-Cbz | 1,3- | 1,3- |
| | BuPh | BuPh | | di-t- | di-t- |
| | | | | BuPh | BuPh |
| 143359 | 1,3- | 1,3- | t-Bu | H | H |
| | di-t- | di-t- | | | |
| | BuPh | BuPh | | | |
| 143360 | 1,3- | 1,3- | Ph | H | H |
| | di-t- | di-t- | | | |
| | BuPh | BuPh | | | |
| 143361 | 1,3- | 1,3- | CN | H | H |
| | di-t- | di-t- | | | |
| | BuPh | BuPh | | | |
| 143362 | 1,3- | 1,3- | DPA | H | H |
| | di-t- | di-t- | | | |
| | BuPh | BuPh | | | |
| 143363 | 1,3- | 1,3- | 2- | H | H |
| | di-t- | di-t- | DBF | | |
| | BuPh | BuPh | | | |
| 143364 | 1,3- | 1,3- | 2- | H | H |
| | di-t- | di-t- | DBT | | |
| | BuPh | BuPh | | | |
| 143365 | 1,3- | 1,3- | 4-t- | H | H |
| | di-t- | di-t- | BuPh | | |
| | BuPh | BuPh | | | |
| 143366 | 1,3- | 1,3- | 9-Cbz | H | H |
| | di-t- | di-t- | | | |
| | BuPh | BuPh | | | |
| 143367 | 1,3- | 1,3- | t-Bu | t-Bu | t-Bu |
| | di-t- | di-t- | | | |
| | BuPh | BuPh | | | |
| 143368 | 1,3- | 1,3- | Ph | t-Bu | t-Bu |
| | di-t- | di-t- | | | |
| | BuPh | BuPh | | | |
| 143369 | 1,3- | 1,3- | CN | t-Bu | t-Bu |
| | di-t- | di-t- | | | |
| | BuPh | BuPh | | | |
| 143370 | 1,3- | 1,3- | DPA | t-Bu | t-Bu |
| | di-t- | di-t- | | | |
| | BuPh | BuPh | | | |
| 143371 | 1,3- | 1,3- | 2- | t-Bu | t-Bu |
| | di-t- | di-t- | DBF | | |
| | BuPh | BuPh | | | |
| 143372 | 1,3- | 1,3- | 2- | t-Bu | t-Bu |
| | di-t- | di-t- | DBT | | |
| | BuPh | BuPh | | | |
| 143373 | 1,3- | 1,3- | 4-t- | t-Bu | t-Bu |
| | di-t- | di-t- | BuPh | | |
| | BuPh | BuPh | | | |
| 143374 | 1,3- | 1,3- | 1,3- | t-Bu | t-Bu |
| | di-t- | di-t- | di-t- | | |
| | BuPh | BuPh | BuPh | | |
| 143375 | 1,3- | 1,3- | 9-Cbz | t-Bu | t-Bu |
| | di-t- | di-t- | | | |
| | BuPh | BuPh | | | |
| 143376 | 1,3- | 1,3- | t-Bu | Ph | Ph |
| | di-t- | di-t- | | | |
| | BuPh | BuPh | | | |
| 143377 | 1,3- | 1,3- | Ph | Ph | Ph |
| | di-t- | di-t- | | | |
| | BuPh | BuPh | | | |
| 143378 | 1,3- | 1,3- | CN | Ph | Ph |
| | di-t- | di-t- | | | |
| | BuPh | BuPh | | | |
| 143379 | 1,3- | 1,3- | DPA | Ph | Ph |
| | di-t- | di-t- | | | |
| | BuPh | BuPh | | | |
| 143380 | 1,3- | 1,3- | 2- | Ph | Ph |
| | di-t- | di-t- | DBF | | |
| | BuPh | BuPh | | | |
| 143381 | 1,3- | 1,3- | 2- | Ph | Ph |
| | di-t- | di-t- | DBT | | |
| | BuPh | BuPh | | | |
| 143382 | 1,3- | 1,3- | 4-t- | Ph | Ph |
| | di-t- | di-t- | BuPh | | |
| | BuPh | BuPh | | | |
| 143383 | 1,3- | 1,3- | 1,3- | Ph | Ph |
| | di-t- | di-t- | di-t- | | |
| | BuPh | BuPh | BuPh | | |
| 143384 | 1,3- | 1,3- | 9-Cbz | Ph | Ph |
| | di-t- | di-t- | | | |
| | BuPh | BuPh | | | |
| 143385 | 1,3- | 1,3- | t-Bu | CN | CN |
| | di-t- | di-t- | | | |
| | BuPh | BuPh | | | |
| 143386 | 1,3- | 1,3- | Ph | CN | CN |
| | di-t- | di-t- | | | |
| | BuPh | BuPh | | | |
| 143387 | 1,3- | 1,3- | CN | CN | CN |
| | di-t- | di-t- | | | |
| | BuPh | BuPh | | | |
| 143388 | 1,3- | 1,3- | DPA | CN | CN |
| | di-t- | di-t- | | | |
| | BuPh | BuPh | | | |
| 143389 | 1,3- | 1,3- | 2- | CN | CN |
| | di-t- | di-t- | DBF | | |
| | BuPh | BuPh | | | |
| 143390 | 1,3- | 1,3- | 2- | CN | CN |
| | di-t- | di-t- | DBT | | |
| | BuPh | BuPh | | | |
| 143391 | 1,3- | 1,3- | 4-t- | CN | CN |
| | di-t- | di-t- | BuPh | | |
| | BuPh | BuPh | | | |
| 143392 | 1,3- | 1,3- | 1,3- | CN | CN |
| | di-t- | di-t- | di-t- | | |
| | BuPh | BuPh | BuPh | | |
| 143393 | 1,3- | 1,3- | 9-Cbz | CN | CN |
| | di-t- | di-t- | | | |
| | BuPh | BuPh | | | |
| 143394 | 1,3- | 1,3- | t-Bu | 9-Cbz | 9-Cbz |
| | di-t- | di-t- | | | |
| | BuPh | BuPh | | | |
| 143395 | 1,3- | 1,3- | Ph | 9-Cbz | 9-Cbz |
| | di-t- | di-t- | | | |
| | BuPh | BuPh | | | |
| 143396 | 1,3- | 1,3- | CN | 9-Cbz | 9-Cbz |
| | di-t- | di-t- | | | |
| | BuPh | BuPh | | | |
| 143397 | 1,3- | 1,3- | DPA | 9-Cbz | 9-Cbz |
| | di-t- | di-t- | | | |
| | BuPh | BuPh | | | |
| 143398 | 1,3- | 1,3- | 2- | 9-Cbz | 9-Cbz |
| | di-t- | di-t- | DBF | | |
| | BuPh | BuPh | | | |
| 143399 | 1,3- | 1,3- | 2- | 9-Cbz | 9-Cbz |
| | di-t- | di-t- | DBT | | |
| | BuPh | BuPh | | | |
| 143400 | 1,3- | 1,3- | 4-t- | 9-Cbz | 9-Cbz |
| | di-t- | di-t- | BuPh | | |
| | BuPh | BuPh | | | |
| 143401 | 1,3- | 1,3- | 1,3- | 9-Cbz | 9-Cbz |
| | di-t- | di-t- | di-t- | | |
| | BuPh | BuPh | BuPh | | |
| 143402 | 1,3- | 1,3- | 9-Cbz | 9-Cbz | 9-Cbz |
| | di-t- | di-t- | | | |
| | BuPh | BuPh | | | |
| 143403 | 1,3- | 1,3- | t-Bu | 4-t- | 4-t- |
| | di-t- | di-t- | | BuPh | BuPh |
| | BuPh | BuPh | | | |
| 143404 | 1,3- | 1,3- | Ph | 4-t- | 4-t- |
| | di-t- | di-t- | | BuPh | BuPh |
| | BuPh | BuPh | | | |
| 143405 | 1,3- | 1,3- | CN | 4-t- | 4-t- |
| | di-t- | di-t- | | BuPh | BuPh |
| | BuPh | BuPh | | | |
| 143406 | 1,3- | 1,3- | DPA | 4-t- | 4-t- |
| | di-t- | di-t- | | BuPh | BuPh |
| | BuPh | BuPh | | | |
| 143407 | 1,3- | 1,3- | 2- | 4-t- | 4-t- |
| | di-t- | di-t- | DBF | BuPh | BuPh |
| | BuPh | BuPh | | | |
| 143408 | 1,3- | 1,3- | 2- | 4-t- | 4-t- |
| | di-t- | di-t- | DBT | BuPh | BuPh |
| | BuPh | BuPh | | | |
| 143409 | 1,3- | 1,3- | 4-t- | 4-t- | 4-t- |
| | di-t- | di-t- | BuPh | BuPh | BuPh |
| | BuPh | BuPh | | | |
| 143410 | 1,3- | 1,3- | 1,3- | 4-t- | 4-t- |
| | di-t- | di-t- | di-t- | BuPh | BuPh |
| | BuPh | BuPh | BuPh | | |
| 143411 | 1,3- | 1,3- | 9-Cbz | 4-t- | 4-t- |
| | di-t- | di-t- | | BuPh | BuPh |
| | BuPh | BuPh | | | |
| 143412 | 1,3- | 1,3- | t-Bu | 1,3- | 1,3- |
| | di-t- | di-t- | | dkt- | di-t- |
| | BuPh | BuPh | | BuPh | BuPh |
| 143413 | 1,3- | 1,3- | Ph | 1,3- | 1,3- |
| | di-t- | di-t- | | di-t- | di-t- |
| | BuPh | BuPh | | BuPh | BuPh |
| 143414 | 1,3- | 1,3- | CN | 1,3- | 1,3- |
| | di-t- | di-t- | | di-t- | di-t- |
| | BuPh | BuPh | | BuPh | BuPh |
| 143415 | 1,3- | 1,3- | DPA | 1,3- | 1,3- |
| | di-t- | di-t- | | di-t- | di-t- |
| | BuPh | BuPh | | BuPh | BuPh |
| 143416 | 1,3- | 1,3- | 2- | 1,3- | 1,3- |
| | di-t- | di-t- | DBF | di-t- | di-t- |
| | BuPh | BuPh | | BuPh | BuPh |
| 143417 | 1,3- | 1,3- | 2- | 1,3- | 1,3- |
| | di-t- | di-t- | DBT | di-t- | di-t- |
| | BuPh | BuPh | | BuPh | BuPh |
| 143418 | 1,3- | 1,3- | 4-t- | 1,3- | 1,3- |
| | di-t- | di-t- | BuPh | di-t- | di-t- |
| | BuPh | BuPh | | BuPh | BuPh |
| 143419 | 1,3- | 1,3- | 1,3- | 1,3- | 1,3- |
| | di-t- | di-t- | di-t- | di-t- | di-t- |
| | BuPh | BuPh | BuPh | BuPh | BuPh |
| 143420 | 1,3- | 1,3- | 9-Cbz | 1,3- | 1,3- |
| | di-t- | di-t- | | di-t- | di-t- |
| | BuPh | BuPh | | BuPh | BuPh |

### Group HC8

The heterocyclic compound may be represented by Formula 15-4, and substituents may be defined as shown in Table 8.

TABLE 8

| Compound No. | R_{11b} | R_{12c} | R_{15C} | R_{18b} | R_{19b} |
|---|---|---|---|---|---|
| 154001 | t-Bu | t-Bu | t-Bu | t-Bu | t-Bu |
| 154002 | Ph | Ph | t-Bu | Ph | Ph |
| 154003 | t-Bu | t-Bu | H | H | H |
| 154004 | H | H | t-Bu | H | H |
| 154005 | H | H | H | t-Bu | t-Bu |
| 154006 | t-Bu | t-Bu | t-Bu | H | H |
| 154007 | H | H | t-Bu | t-Bu | t-Bu |
| 154008 | t-Bu | t-Bu | H | t-Bu | t-Bu |
| 154009 | Ph | Ph | H | H | H |
| 154010 | H | H | Ph | H | H |
| 154011 | H | H | H | Ph | Ph |
| 154012 | Ph | Ph | Ph | H | H |
| 154013 | H | H | Ph | Ph | Ph |
| 154014 | Ph | Ph | H | Ph | Ph |
| 154015 | CN | CN | H | H | H |
| 154016 | H | H | CN | H | H |
| 154017 | H | H | H | CN | CN |
| 154018 | CN | CN | CN | H | H |
| 154019 | H | H | CN | CN | CN |
| 154020 | CN | CN | H | CN | CN |
| 154021 | 9-Cbz | 9-Cbz | H | H | H |
| 154022 | H | H | 9-Cbz | H | H |
| 154023 | H | H | H | 9-Cbz | 9-Cbz |
| 154024 | 9-Cbz | 9-Cbz | 9-Cbz | H | H |
| 154025 | H | H | 9-Cbz | 9-Cbz | 9-Cbz |
| 154026 | 9-Cbz | 9-Cbz | H | 9-Cbz | 9-Cbz |
| 154027 | Me | Me | H | H | H |
| 154028 | H | H | Me | H | H |
| 154029 | H | H | H | Me | Me |
| 154030 | Me | Me | Me | H | H |
| 154031 | H | H | Me | Me | Me |
| 154032 | Me | Me | H | Me | Me |
| 154033 | CD₃ | CD3 | H | H | H |
| 154034 | H | H | CD3 | H | H |
| 154035 | H | H | H | CD3 | CD3 |
| 154036 | CD3 | CD3 | CD3 | H | H |
| 154037 | H | H | CD3 | CD3 | CD3 |
| 154038 | CD3 | CD3 | H | CD3 | CD3 |
| 154039 | 4-t-BuPh | 4-t-BuPh | H | H | H |
| 154040 | H | H | 4-t-BuPh | H | H |
| 154041 | H | H | H | 4-t-BuPh | 4-t-BuPh |
| 154042 | 4-t-BuPh | 4-t-BuPh | 4-t-BuPh | H | H |
| 154043 | H | H | 4-t-BuPh | 4-t-BuPh | 4-t-BuPh |
| 154044 | 4-t-BuPh | 4-t-BuPh | H | 4-t-BuPh | 4-t-BuPh |
| 154045 | 1,3-di-t-BuPh | 1,3-di-t-BuPh | H | H | H |
| 154046 | H | H | 1,3-di-t-BuPh | H | H |
| 154047 | H | H | H | 1,3-di-t-BuPh | 1,3-di-t-BuPh |
| 154048 | 1,3-di-t-BuPh | 1,3-di-t-BuPh | 1,3-di-t-BuPh | H | H |
| 154049 | H | H | 1,3-di-t-BuPh | 1,3-di-t-BuPh | 1,3-di-t-BuPh |
| 154050 | 1,3-di-t-BuPh | 1,3-di-t-BuPh | H | 1,3-di-t-BuPh | 1,3-di-t-BuPh |
| 154051 | t-Bu | t-Bu | Ph | H | H |
| 154052 | t-Bu | t-Bu | CN | H | H |
| 154053 | t-Bu | t-Bu | DPA | H | H |
| 154054 | t-Bu | t-Bu | 2-DBF | H | H |
| 154055 | t-Bu | t-Bu | 2-DBT | H | H |
| 154056 | t-Bu | t-Bu | 4-t-BuPh | H | H |
| 154057 | t-Bu | t-Bu | 1,3-di-t-BuPh | H | H |
| 154058 | t-Bu | t-Bu | 9-Cbz | H | H |
| 154059 | t-Bu | t-Bu | Ph | t-Bu | t-Bu |
| 154060 | t-Bu | t-Bu | CN | t-Bu | t-Bu |
| 154061 | t-Bu | t-Bu | DPA | t-Bu | t-Bu |
| 154062 | t-Bu | t-Bu | 2-DBF | t-Bu | t-Bu |
| 154063 | t-Bu | t-Bu | 2-DBT | t-Bu | t-Bu |
| 154064 | t-Bu | t-Bu | 4-t-BuPh | t-Bu | t-Bu |
| 154065 | t-Bu | t-Bu | 1,3-di-t-BuPh | t-Bu | t-Bu |
| 154066 | t-Bu | t-Bu | 9-Cbz | t-Bu | t-Bu |
| 154067 | t-Bu | t-Bu | t-Bu | Ph | Ph |
| 154068 | t-Bu | t-Bu | Ph | Ph | Ph |
| 154069 | t-Bu | t-Bu | CN | Ph | Ph |
| 154070 | t-Bu | t-Bu | DPA | Ph | Ph |
| 154071 | t-Bu | t-Bu | 2-DBF | Ph | Ph |
| 154072 | t-Bu | t-Bu | 2-DBT | Ph | Ph |
| 154073 | t-Bu | t-Bu | 4-t-BuPh | Ph | Ph |
| 154074 | t-Bu | t-Bu | 1,3-di-t-BuPh | Ph | Ph |
| 154075 | t-Bu | t-Bu | 9-Cbz | Ph | Ph |
| 154076 | t-Bu | t-Bu | t-Bu | CN | CN |
| 154077 | t-Bu | t-Bu | Ph | CN | CN |
| 154078 | t-Bu | t-Bu | CN | CN | CN |
| 154079 | t-Bu | t-Bu | DPA | CN | CN |
| 154080 | t-Bu | t-Bu | 2-DBF | CN | CN |
| 154081 | t-Bu | t-Bu | 2-DBT | CN | CN |
| 154082 | t-Bu | t-Bu | 4-t-BuPh | CN | CN |
| 154083 | t-Bu | t-Bu | 1,3-di-t-BuPh | CN | CN |
| 154084 | t-Bu | t-Bu | 9-Cbz | CN | CN |
| 154085 | t-Bu | t-Bu | t-Bu | 9-Cbz | 9-Cbz |
| 154086 | t-Bu | t-Bu | Ph | 9-Cbz | 9-Cbz |
| 154087 | t-Bu | t-Bu | CN | 9-Cbz | 9-Cbz |
| 154088 | t-Bu | t-Bu | DPA | 9-Cbz | 9-Cbz |
| 154089 | t-Bu | t-Bu | 2-DBF | 9-Cbz | 9-Cbz |
| 154090 | t-Bu | t-Bu | 2-DBT | 9-Cbz | 9-Cbz |
| 154091 | t-Bu | t-Bu | 4-t-BuPh | 9-Cbz | 9-Cbz |
| 154092 | t-Bu | t-Bu | 1,3-di-t-BuPh | 9-Cbz | 9-Cbz |
| 154093 | t-Bu | t-Bu | 9-Cbz | 9-Cbz | 9-Cbz |
| 154094 | t-Bu | t-Bu | t-Bu | 4-t-BuPh | 4-t-BuPh |
| 154095 | t-Bu | t-Bu | Ph | 4-t-BuPh | 4-t-BuPh |
| 154096 | t-Bu | t-Bu | CN | 4-t-BuPh | 4-t-BuPh |
| 154097 | t-Bu | t-Bu | DPA | 4-t-BuPh | 4-t-BuPh |
| 154098 | t-Bu | t-Bu | 2-DBF | 4-t-BuPh | 4-t-BuPh |
| 154099 | t-Bu | t-Bu | 2-DBT | 4-t-BuPh | 4-t-BuPh |
| 154100 | t-Bu | t-Bu | 4-t-BuPh | 4-t-BuPh | 4-t-BuPh |
| 154101 | t-Bu | t-Bu | 1,3-di-t-BuPh | 4-t-BuPh | 4-t-BuPh |
| 154102 | t-Bu | t-Bu | 9-Cbz | 4-t-BuPh | 4-t-BuPh |
| 154103 | t-Bu | t-Bu | t-Bu | 1,3-di-t-BuPh | 1,3-di-t-BuPh |
| 154104 | t-Bu | t-Bu | Ph | 1,3-di-t-BuPh | 1,3-di-t-BuPh |
| 154105 | t-Bu | t-Bu | CN | 1,3-di-t-BuPh | 1,3-di-t-BuPh |
| 154106 | t-Bu | t-Bu | DPA | 1,3-di-t-BuPh | 1,3-di-t-BuPh |
| 154107 | t-Bu | t-Bu | 2-DBF | 1,3-di-t-BuPh | 1,3-di-t-BuPh |
| 154108 | t-Bu | t-Bu | 2-DBT | 1,3-di-t-BuPh | 1,3-di-t-BuPh |
| 154109 | t-Bu | t-Bu | 4-t-BuPh | 1,3-di-t-BuPh | 1,3-di-t-BuPh |
| 154110 | t-Bu | t-Bu | 1,3-di-t-BuPh | 1,3-di-t-BuPh | 1,3-di-t-BuPh |
| 154111 | t-Bu | t-Bu | 9-Cbz | 1,3-di-t-BuPh | 1,3-di-t-BuPh |
| 154112 | Ph | Ph | t-Bu | H | H |
| 154113 | Ph | Ph | CN | H | H |
| 154114 | Ph | Ph | DPA | H | H |
| 154115 | Ph | Ph | 2-DBF | H | H |
| 154116 | Ph | Ph | 2-DBT | H | H |
| 154117 | Ph | Ph | 4-t-BuPh | H | H |
| 154118 | Ph | Ph | 1,3-di-t-BuPh | H | H |
| 154119 | Ph | Ph | 9-Cbz | H | H |
| 154120 | Ph | Ph | t-Bu | t-Bu | t-Bu |
| 154121 | Ph | Ph | Ph | t-Bu | t-Bu |
| 154122 | Ph | Ph | CN | t-Bu | t-Bu |
| 154123 | Ph | Ph | DPA | t-Bu | t-Bu |
| 154124 | Ph | Ph | 2-DBF | t-Bu | t-Bu |
| 154125 | Ph | Ph | 2-DBT | t-Bu | t-Bu |
| 154126 | Ph | Ph | 4-t-BuPh | t-Bu | t-Bu |
| 154127 | Ph | Ph | 1,3-di-t-BuPh | t-Bu | t-Bu |
| 154128 | Ph | Ph | 9-Cbz | t-Bu | t-Bu |
| 154129 | Ph | Ph | Ph | Ph | Ph |
| 154130 | Ph | Ph | CN | Ph | Ph |
| 154131 | Ph | Ph | DPA | Ph | Ph |
| 154132 | Ph | Ph | 2-DBF | Ph | Ph |
| 154133 | Ph | Ph | 2-DBT | Ph | Ph |
| 154134 | Ph | Ph | 4-t-BuPh | Ph | Ph |
| 154135 | Ph | Ph | 1,3-di-t-BuPh | Ph | Ph |
| 154136 | Ph | Ph | 9-Cbz | Ph | Ph |
| 154137 | Ph | Ph | t-Bu | CN | CN |
| 154138 | Ph | Ph | Ph | CN | CN |
| 154139 | Ph | Ph | CN | CN | CN |
| 154140 | Ph | Ph | DPA | CN | CN |
| 154141 | Ph | Ph | 2-DBF | CN | CN |
| 154142 | Ph | Ph | 2-DBT | CN | CN |
| 154143 | Ph | Ph | 4-t-BuPh | CN | CN |
| 154144 | Ph | Ph | 1,3-di-t-BuPh | CN | CN |
| 154145 | Ph | Ph | 9-Cbz | CN | CN |
| 154146 | Ph | Ph | t-Bu | 9-Cbz | 9-Cbz |
| 154147 | Ph | Ph | Ph | 9-Cbz | 9-Cbz |
| 154148 | Ph | Ph | CN | 9-Cbz | 9-Cbz |
| 154149 | Ph | Ph | DPA | 9-Cbz | 9-Cbz |
| 154150 | Ph | Ph | 2-DBF | 9-Cbz | 9-Cbz |
| 154151 | Ph | Ph | 2-DBT | 9-Cbz | 9-Cbz |
| 154152 | Ph | Ph | 4-t-BuPh | 9-Cbz | 9-Cbz |
| 154153 | Ph | Ph | 1,3-di-t-BuPh | 9-Cbz | 9-Cbz |
| 154154 | Ph | Ph | 9-Cbz | 9-Cbz | 9-Cbz |
| 154155 | Ph | Ph | t-Bu | 4-t-BuPh | 4-t-BuPh |
| 154156 | Ph | Ph | Ph | 4-t-BuPh | 4-t-BuPh |
| 154157 | Ph | Ph | CN | 4-t-BuPh | 4-t-BuPh |
| 154158 | Ph | Ph | DPA | 4-t-BuPh | 4-t-BuPh |
| 154159 | Ph | Ph | 2-DBF | 4-t-BuPh | 4-t-BuPh |
| 154160 | Ph | Ph | 2-DBT | 4-t-BuPh | 4-t-BuPh |
| 154161 | Ph | Ph | 4-t-BuPh | 4-t-BuPh | 4-t-BuPh |
| 154162 | Ph | Ph | 1,3-di-t-BuPh | 4-t-BuPh | 4-t-BuPh |
| 154163 | Ph | Ph | 9-Cbz | 4-t-BuPh | 4-t-BuPh |
| 154164 | Ph | Ph | t-Bu | 1,3-di-t-BuPh | 1,3-di-t-BuPh |
| 154165 | Ph | Ph | Ph | 1,3-di-t-BuPh | 1,3-di-t-BuPh |
| 154166 | Ph | Ph | CN | 1,3-di-t-BuPh | 1,3-di-t-BuPh |
| 154167 | Ph | Ph | DPA | 1,3-di-t-BuPh | 1,3-di-t-BuPh |
| 154168 | Ph | Ph | 2-DBF | 1,3-di-t-BuPh | 1,3-di-t-BuPh |
| 154169 | Ph | Ph | 2-DBT | 1,3-di-t-BuPh | 1,3-di-t-BuPh |
| 154170 | Ph | Ph | 4-t-BuPh | 1,3-di-t-BuPh | 1,3-di-t-BuPh |
| 154171 | Ph | Ph | 1,3-di-t-BuPh | 1,3-di-t-BuPh | 1,3-di-t-BuPh |
| 154172 | Ph | Ph | 9-Cbz | 1,3-di-t-BuPh | 1,3-di-t-BuPh |
| 154173 | 9-Cbz | 9-Cbz | t-Bu | H | H |
| 154174 | 9-Cbz | 9-Cbz | Ph | H | H |
| 154175 | 9-Cbz | 9-Cbz | CN | H | H |
| 154176 | 9-Cbz | 9-Cbz | DPA | H | H |
| 154177 | 9-Cbz | 9-Cbz | 2-DBF | H | H |
| 154178 | 9-Cbz | 9-Cbz | 2-DBT | H | H |
| 154179 | 9-Cbz | 9-Cbz | 4-t-BuPh | H | H |
| 154180 | 9-Cbz | 9-Cbz | 1,3-di-t-BuPh | H | H |
| 154181 | 9-Cbz | 9-Cbz | t-Bu | t-Bu | t-Bu |
| 154182 | 9-Cbz | 9-Cbz | Ph | t-Bu | t-Bu |
| 154183 | 9-Cbz | 9-Cbz | CN | t-Bu | t-Bu |
| 154184 | 9-Cbz | 9-Cbz | DPA | t-Bu | t-Bu |
| 154185 | 9-Cbz | 9-Cbz | 2-DBF | t-Bu | t-Bu |
| 154186 | 9-Cbz | 9-Cbz | 2-DBT | t-Bu | t-Bu |
| 154187 | 9-Cbz | 9-Cbz | 4-t-BuPh | t-Bu | t-Bu |
| 154188 | 9-Cbz | 9-Cbz | 1,3-di-t-BuPh | t-Bu | t-Bu |
| 154189 | 9-Cbz | 9-Cbz | 9-Cbz | t-Bu | t-Bu |
| 154190 | 9-Cbz | 9-Cbz | t-Bu | Ph | Ph |
| 154191 | 9-Cbz | 9-Cbz | Ph | Ph | Ph |
| 154192 | 9-Cbz | 9-Cbz | CN | Ph | Ph |
| 154193 | 9-Cbz | 9-Cbz | DPA | Ph | Ph |
| 154194 | 9-Cbz | 9-Cbz | 2-DBF | Ph | Ph |
| 154195 | 9-Cbz | 9-Cbz | 2-DBT | Ph | Ph |
| 154196 | 9-Cbz | 9-Cbz | 4-t-BuPh | Ph | Ph |
| 154197 | 9-Cbz | 9-Cbz | 1,3-di-t-BuPh | Ph | Ph |
| 154198 | 9-Cbz | 9-Cbz | 9-Cbz | Ph | Ph |
| 154199 | 9-Cbz | 9-Cbz | t-Bu | CN | CN |
| 154200 | 9-Cbz | 9-Cbz | Ph | CN | CN |
| 154201 | 9-Cbz | 9-Cbz | CN | CN | CN |
| 154202 | 9-Cbz | 9-Cbz | DPA | CN | CN |
| 154203 | 9-Cbz | 9-Cbz | 2-DBF | CN | CN |
| 154204 | 9-Cbz | 9-Cbz | 2-DBT | CN | CN |
| 154205 | 9-Cbz | 9-Cbz | 4-t-BuPh | CN | CN |
| 154206 | 9-Cbz | 9-Cbz | 1,3-di-t-BuPh | CN | CN |
| 154207 | 9-Cbz | 9-Cbz | 9-Cbz | CN | CN |
| 154208 | 9-Cbz | 9-Cbz | t-Bu | 9-Cbz | 9-Cbz |
| 154209 | 9-Cbz | 9-Cbz | Ph | 9-Cbz | 9-Cbz |
| 154210 | 9-Cbz | 9-Cbz | CN | 9-Cbz | 9-Cbz |
| 154211 | 9-Cbz | 9-Cbz | DPA | 9-Cbz | 9-Cbz |
| 154212 | 9-Cbz | 9-Cbz | 2-DBF | 9-Cbz | 9-Cbz |
| 154213 | 9-Cbz | 9-Cbz | 2-DBT | 9-Cbz | 9-Cbz |
| 154214 | 9-Cbz | 9-Cbz | 4-t-BuPh | 9-Cbz | 9-Cbz |
| 154215 | 9-Cbz | 9-Cbz | 1,3-di-t-BuPh | 9-Cbz | 9-Cbz |
| 154216 | 9-Cbz | 9-Cbz | 9-Cbz | 9-Cbz | 9-Cbz |
| 154217 | 9-Cbz | 9-Cbz | t-Bu | 4-t-BuPh | 4-t-BuPh |
| 154218 | 9-Cbz | 9-Cbz | Ph | 4-t-BuPh | 4-t-BuPh |
| 154219 | 9-Cbz | 9-Cbz | CN | 4-t-BuPh | 4-t-BuPh |
| 154220 | 9-Cbz | 9-Cbz | DPA | 4-t-BuPh | 4-t-BuPh |
| 154221 | 9-Cbz | 9-Cbz | 2-DBF | 4-t-BuPh | 4-t-BuPh |
| 154222 | 9-Cbz | 9-Cbz | 2-DBT | 4-t-BuPh | 4-t-BuPh |
| 154223 | 9-Cbz | 9-Cbz | 4-t-BuPh | 4-t-BuPh | 4-t-BuPh |
| 154224 | 9-Cbz | 9-Cbz | 1,3-di-t-BuPh | 4-t-BuPh | 4-t-BuPh |
| 154225 | 9-Cbz | 9-Cbz | 9-Cbz | 4-t-BuPh | 4-t-BuPh |
| 154226 | 9-Cbz | 9-Cbz | t-Bu | 1,3-di-t-BuPh | 1,3-di-t-BuPh |
| 154227 | 9-Cbz | 9-Cbz | Ph | 1,3-di-t-BuPh | 1,3-di-t-BuPh |
| 154228 | 9-Cbz | 9-Cbz | CN | 1,3-di-t-BuPh | 1,3-di-t-BuPh |
| 154229 | 9-Cbz | 9-Cbz | DPA | 1,3-di-t-BuPh | 1,3-di-t-BuPh |
| 154230 | 9-Cbz | 9-Cbz | 2-DBF | 1,3-di-t-BuPh | 1,3-di-t-BuPh |
| 154231 | 9-Cbz | 9-Cbz | 2-DBT | 1,3-di-t-BuPh | 1,3-di-t-BuPh |
| 154232 | 9-Cbz | 9-Cbz | 4-t-BuPh | 1,3-di-t-BuPh | 1,3-di-t-BuPh |
| 154233 | 9-Cbz | 9-Cbz | 1,3-di-t-BuPh | 1,3-di-t-BuPh | 1,3-di-t-BuPh |
| 154234 | 9-Cbz | 9-Cbz | 9-Cbz | 1,3-di-t-BuPh | 1,3-di-t-BuPh |
| 154235 | CN | CN | t-Bu | H | H |
| 154236 | CN | CN | Ph | H | H |
| 154237 | CN | CN | DPA | H | H |
| 154238 | CN | CN | 2-DBF | H | H |
| 154239 | CN | CN | 2-DBT | H | H |
| 154240 | CN | CN | 4-t-BuPh | H | H |
| 154241 | CN | CN | 1,3-di-t-BuPh | H | H |
| 154242 | CN | CN | 9-Cbz | H | H |
| 154243 | CN | CN | t-Bu | t-Bu | t-Bu |
| 154244 | CN | CN | Ph | t-Bu | t-Bu |
| 154245 | CN | CN | CN | t-Bu | t-Bu |
| 154246 | CN | CN | DPA | t-Bu | t-Bu |
| 154247 | CN | CN | 2-DBF | t-Bu | t-Bu |
| 154248 | CN | CN | 2-DBT | t-Bu | t-Bu |
| 154249 | CN | CN | 4-t-BuPh | t-Bu | t-Bu |
| 154250 | CN | CN | 1,3-di-t-BuPh | t-Bu | t-Bu |
| 154251 | CN | CN | 9-Cbz | t-Bu | t-Bu |
| 154252 | CN | CN | t-Bu | Ph | Ph |
| 154253 | CN | CN | Ph | Ph | Ph |
| 154254 | CN | CN | CN | Ph | Ph |
| 154255 | CN | CN | DPA | Ph | Ph |
| 154256 | CN | CN | 2-DBF | Ph | Ph |
| 154257 | CN | CN | 2-DBT | Ph | Ph |
| 154258 | CN | CN | 4-t-BuPh | Ph | Ph |
| 154259 | CN | CN | 1,3-di-t-BuPh | Ph | Ph |
| 154260 | CN | CN | 9-Cbz | Ph | Ph |
| 154261 | CN | CN | t-Bu | CN | CN |
| 154262 | CN | CN | Ph | CN | CN |
| 154263 | CN | CN | CN | CN | CN |
| 154264 | CN | CN | DPA | CN | CN |
| 154265 | CN | CN | 2-DBF | CN | CN |
| 154266 | CN | CN | 2-DBT | CN | CN |
| 154267 | CN | CN | 4-t-BuPh | CN | CN |
| 154268 | CN | CN | 1,3-di-t-BuPh | CN | CN |
| 154269 | CN | CN | 9-Cbz | CN | CN |
| 154270 | CN | CN | t-Bu | 9-Cbz | 9-Cbz |
| 154271 | CN | CN | Ph | 9-Cbz | 9-Cbz |
| 154272 | CN | CN | CN | 9-Cbz | 9-Cbz |
| 154273 | CN | CN | DPA | 9-Cbz | 9-Cbz |
| 154274 | CN | CN | 2-DBF | 9-Cbz | 9-Cbz |
| 154275 | CN | CN | 2-DBT | 9-Cbz | 9-Cbz |
| 154276 | CN | CN | 4-t-BuPh | 9-Cbz | 9-Cbz |
| 154277 | CN | CN | 1,3-di-t-BuPh | 9-Cbz | 9-Cbz |
| 154278 | CN | CN | 9-Cbz | 9-Cbz | 9-Cbz |
| 154279 | CN | CN | t-Bu | 4-t-BuPh | 4-t-BuPh |
| 154280 | CN | CN | Ph | 4-t-BuPh | 4-t-BuPh |
| 154281 | CN | CN | CN | 4-t-BuPh | 4-t-BuPh |
| 154282 | CN | CN | DPA | 4-t-BuPh | 4-t-BuPh |
| 154283 | CN | CN | 2-DBF | 4-t-BuPh | 4-t-BuPh |
| 154284 | CN | CN | 2-DBT | 4-t-BuPh | 4-t-BuPh |
| 154285 | CN | CN | 4-t-BuPh | 4-t-BuPh | 4-t-BuPh |
| 154286 | CN | CN | 1,3-di-t-BuPh | 4-t-BuPh | 4-t-BuPh |
| 154287 | CN | CN | 9-Cbz | 4-t-BuPh | 4-t-BuPh |
| 154288 | CN | CN | t-Bu | 1,3-di-t-BuPh | 1,3-di-t-BuPh |
| 154289 | CN | CN | Ph | 1,3-di-t-BuPh | 1,3-di-t-BuPh |
| 154290 | CN | CN | CN | 1,3-di-t-BuPh | 1,3-di-t-BuPh |
| 154291 | CN | CN | DPA | 1,3-di-t-BuPh | 1,3-di-t-BuPh |
| 154292 | CN | CN | 2-DBF | 1,3-di-t-BuPh | 1,3-di-t-BuPh |
| 154293 | CN | CN | 2-DBT | 1,3-di-t-BuPh | 1,3-di-t-BuPh |
| 154294 | CN | CN | 4-t-BuPh | 1,3-di-t-BuPh | 1,3-di-t-BuPh |
| 154295 | CN | CN | 1,3-di-t-BuPh | 1,3-di-t-BuPh | 1,3-di-t-BuPh |
| 154296 | CN | CN | 9-Cbz | 1,3-di-t-BuPh | 1,3-di-t-BuPh |
| 154297 | 4-t-BuPh | 4-t-BuPh | t-Bu | H | H |
| 154298 | 4-t-BuPh | 4-t-BuPh | Ph | H | H |
| 154299 | 4-t-BuPh | 4-t-BuPh | CN | H | H |
| 154300 | 4-t-BuPh | 4-t-BuPh | DPA | H | H |
| 154301 | 4-t-BuPh | 4-t-BuPh | 2-DBF | H | H |
| 154302 | 4-t-BuPh | 4-t-BuPh | 2-DBT | H | H |
| 154303 | 4-t-BuPh | 4-t-BuPh | 1,3-di-t-BuPh | H | H |
| 154304 | 4-t-BuPh | 4-t-BuPh | 9-Cbz | H | H |
| 154305 | 4-t-BuPh | 4-t-BuPh | t-Bu | t-Bu | t-Bu |
| 154306 | 4-t-BuPh | 4-t-BuPh | Ph | t-Bu | t-Bu |
| 154307 | 4-t-BuPh | 4-t-BuPh | CN | t-Bu | t-Bu |
| 154308 | 4-t-BuPh | 4-t-BuPh | DPA | t-Bu | t-Bu |
| 154309 | 4-t-BuPh | 4-t-BuPh | 2-DBF | t-Bu | t-Bu |
| 154310 | 4-t-BuPh | 4-t-BuPh | 2-DBT | t-Bu | t-Bu |
| 154311 | 4-t-BuPh | 4-t-BuPh | 4-t-BuPh | t-Bu | t-Bu |
| 154312 | 4-t-BuPh | 4-t-BuPh | 1,3-di-t-BuPh | t-Bu | t-Bu |
| 154313 | 4-t-BuPh | 4-t-BuPh | 9-Cbz | t-Bu | t-Bu |
| 154314 | 4-t-BuPh | 4-t-BuPh | t-Bu | Ph | Ph |
| 154315 | 4-t-BuPh | 4-t-BuPh | Ph | Ph | Ph |
| 154316 | 4-t-BuPh | 4-t-BuPh | CN | Ph | Ph |
| 154317 | 4-t-BuPh | 4-t-BuPh | DPA | Ph | Ph |
| 154318 | 4-t-BuPh | 4-t-BuPh | 2-DBF | Ph | Ph |
| 154319 | 4-t-BuPh | 4-t-BuPh | 2-DBT | Ph | Ph |
| 154320 | 4-t-BuPh | 4-t-BuPh | 4-t-BuPh | Ph | Ph |
| 154321 | 4-t-BuPh | 4-t-BuPh | 1,3-di-t-BuPh | Ph | Ph |
| 154322 | 4-t-BuPh | 4-t-BuPh | 9-Cbz | Ph | Ph |
| 154323 | 4-t-BuPh | 4-t-BuPh | t-Bu | CN | CN |
| 154324 | 4-t-BuPh | 4-t-BuPh | Ph | CN | CN |
| 154325 | 4-t-BuPh | 4-t-BuPh | CN | CN | CN |
| 154326 | 4-t-BuPh | 4-t-BuPh | DPA | CN | CN |
| 154327 | 4-t-BuPh | 4-t-BuPh | 2-DBF | CN | CN |
| 154328 | 4-t-BuPh | 4-t-BuPh | 2-DBT | CN | CN |
| 154329 | 4-t-BuPh | 4-t-BuPh | 4-t-BuPh | CN | CN |
| 154330 | 4-t-BuPh | 4-t-BuPh | 1,3-di-t-BuPh | CN | CN |
| 154331 | 4-t-BuPh | 4-t-BuPh | 9-Cbz | CN | CN |
| 154332 | 4-t-BuPh | 4-t-BuPh | t-Bu | 9-Cbz | 9-Cbz |
| 154333 | 4-t-BuPh | 4-t-BuPh | Ph | 9-Cbz | 9-Cbz |
| 154334 | 4-t-BuPh | 4-t-BuPh | CN | 9-Cbz | 9-Cbz |
| 154335 | 4-t-BuPh | 4-t-BuPh | DPA | 9-Cbz | 9-Cbz |
| 154336 | 4-t-BuPh | 4-t-BuPh | 2-DBF | 9-Cbz | 9-Cbz |
| 154337 | 4-t-BuPh | 4-t-BuPh | 2-DBT | 9-Cbz | 9-Cbz |
| 154338 | 4-t-BuPh | 4-t-BuPh | 4-t-BuPh | 9-Cbz | 9-Cbz |
| 154339 | 4-t-BuPh | 4-t-BuPh | 1,3-di-t-BuPh | 9-Cbz | 9-Cbz |
| 154340 | 4-t-BuPh | 4-t-BuPh | 9-Cbz | 9-Cbz | 9-Cbz |
| 154341 | 4-t-BuPh | 4-t-BuPh | t-Bu | 4-t-BuPh | 4-t-BuPh |
| 154342 | 4-t-BuPh | 4-t-BuPh | Ph | 4-t-BuPh | 4-t-BuPh |
| 154343 | 4-t-BuPh | 4-t-BuPh | CN | 4-t-BuPh | 4-t-BuPh |
| 154344 | 4-t-BuPh | 4-t-BuPh | DPA | 4-t-BuPh | 4-t-BuPh |
| 154345 | 4-t-BuPh | 4-t-BuPh | 2-DBF | 4-t-BuPh | 4-t-BuPh |
| 154346 | 4-t-BuPh | 4-t-BuPh | 2-DBT | 4-t-BuPh | 4-t-BuPh |
| 154347 | 4-t-BuPh | 4-t-BuPh | 4-t-BuPh | 4-t-BuPh | 4-t-BuPh |
| 154348 | 4-t-BuPh | 4-t-BuPh | 1,3-di-t-BuPh | 4-t-BuPh | 4-t-BuPh |
| 154349 | 4-t-BuPh | 4-t-BuPh | 9-Cbz | 4-t-BuPh | 4-t-BuPh |
| 154350 | 4-t-BuPh | 4-t-BuPh | t-Bu | 1,3-di-t-BuPh | 1,3-di-t-BuPh |
| 154351 | 4-t-BuPh | 4-t-BuPh | Ph | 1,3-di-t-BuPh | 1,3-di-t-BuPh |
| 154352 | 4-t-BuPh | 4-t-BuPh | CN | 1,3-di-t-BuPh | 1,3-di-t-BuPh |
| 154353 | 4-t-BuPh | 4-t-BuPh | DPA | 1,3-di-t-BuPh | 1,3-di-t-BuPh |
| 154354 | 4-t-BuPh | 4-t-BuPh | 2-DBF | 1,3-di-t-BuPh | 1,3-di-t-BuPh |
| 154355 | 4-t-BuPh | 4-t-BuPh | 2-DBT | 1,3-di-t-BuPh | 1,3-di-t-BuPh |
| 154356 | 4-t-BuPh | 4-t-BuPh | 4-t-BuPh | 1,3-di-t-BuPh | 1,3-di-t-BuPh |
| 154357 | 4-t-BuPh | 4-t-BuPh | 1,3-di-t-BuPh | 1,3-di-t-BuPh | 1,3-di-t-BuPh |
| 154358 | 4-t-BuPh | 4-t-BuPh | 9-Cbz | 1,3-di-t-BuPh | 1,3-di-t-BuPh |
| 154359 | 1,3-di-t-BuPh | 1,3-di-t-BuPh | t-Bu | H | H |
| 154360 | 1,3-di-t-BuPh | 1,3-di-t-BuPh | Ph | H | H |
| 154361 | 1,3-di-t-BuPh | 1,3-di-t-BuPh | CN | H | H |
| 154362 | 1,3-di-t-BuPh | 1,3-di-t-BuPh | DPA | H | H |
| 154363 | 1,3-di-t-BuPh | 1,3-di-t-BuPh | 2-DBF | H | H |
| 154364 | 1,3-di-t-BuPh | 1,3-di-t-BuPh | 2-DBT | H | H |
| 154365 | 1,3-di-t-BuPh | 1,3-di-t-BuPh | 4-t-BuPh | H | H |
| 154366 | 1,3-di-t-BuPh | 1,3-di-t-BuPh | 9-Cbz | H | H |
| 154367 | 1,3-di-t-BuPh | 1,3-di-t-BuPh | t-Bu | t-Bu | t-Bu |
| 154368 | 1,3-di-t-BuPh | 1,3-di-t-BuPh | Ph | t-Bu | t-Bu |
| 154369 | 1,3-di-t-BuPh | 1,3-di-t-BuPh | CN | t-Bu | t-Bu |
| 154370 | 1,3-di-t-BuPh | 1,3-di-t-BuPh | DPA | t-Bu | t-Bu |
| 154371 | 1,3-di-t-BuPh | 1,3-di-t-BuPh | 2-DBF | t-Bu | t-Bu |
| 154372 | 1,3-di-t-BuPh | 1,3-di-t-BuPh | 2-DBT | t-Bu | t-Bu |
| 154373 | 1,3-di-t-BuPh | 1,3-di-t-BuPh | 4-t-BuPh | t-Bu | t-Bu |
| 154374 | 1,3-di-t-BuPh | 1,3-di-t-BuPh | 1,3-di-t-BuPh | t-Bu | t-Bu |
| 154375 | 1,3-di-t-BuPh | 1,3-di-t-BuPh | 9-Cbz | t-Bu | t-Bu |
| 154376 | 1,3-di-t-BuPh | 1,3-di-t-BuPh | t-Bu | Ph | Ph |
| 154377 | 1,3-di-t-BuPh | 1,3-di-t-BuPh | Ph | Ph | Ph |
| 154378 | 1,3-di-t-BuPh | 1,3-di-t-BuPh | CN | Ph | Ph |
| 154379 | 1,3-di-t-BuPh | 1,3-di-t-BuPh | DPA | Ph | Ph |
| 154380 | 1,3-di-t-BuPh | 1,3-di-t-BuPh | 2-DBF | Ph | Ph |
| 154381 | 1,3-di-t-BuPh | 1,3-di-t-BuPh | 2-DBT | Ph | Ph |
| 154382 | 1,3-di-t-BuPh | 1,3-di-t-BuPh | 4-t-BuPh | Ph | Ph |
| 154383 | 1,3-di-t-BuPh | 1,3-di-t-BuPh | 1,3-di-t-BuPh | Ph | Ph |
| 154384 | 1,3-di-t-BuPh | 1,3-di-t-BuPh | 9-Cbz | Ph | Ph |
| 154385 | 1,3-di-t-BuPh | 1,3-di-t-BuPh | t-Bu | CN | CN |
| 154386 | 1,3-di-t-BuPh | 1,3-di-t-BuPh | Ph | CN | CN |
| 154387 | 1,3-di-t-BuPh | 1,3-di-t-BuPh | CN | CN | CN |
| 154388 | 1,3-di-t-BuPh | 1,3-di-t-BuPh | DPA | CN | CN |
| 154389 | 1,3-di-t-BuPh | 1,3-di-t-BuPh | 2-DBF | CN | CN |
| 154390 | 1,3-di-t-BuPh | 1,3-di-t-BuPh | 2-DBT | CN | CN |
| 154391 | 1,3-di-t-BuPh | 1,3-di-t-BuPh | 4-t-BuPh | CN | CN |
| 154392 | 1,3-di-t-BuPh | 1,3-di-t-BuPh | 1,3-di-t-BuPh | CN | CN |
| 154393 | 1,3-di-t-BuPh | 1,3-di-t-BuPh | 9-Cbz | CN | CN |
| 154394 | 1,3-di-t-BuPh | 1,3-di-t-BuPh | t-Bu | 9-Cbz | 9-Cbz |
| 154395 | 1,3-di-t-BuPh | 1,3-di-t-BuPh | Ph | 9-Cbz | 9-Cbz |
| 154396 | 1,3-di-t-BuPh | 1,3-di-t-BuPh | CN | 9-Cbz | 9-Cbz |
| 154397 | 1,3-di-t-BuPh | 1,3-di-t-BuPh | DPA | 9-Cbz | 9-Cbz |
| 154398 | 1,3-di-t-BuPh | 1,3-di-t-BuPh | 2-DBF | 9-Cbz | 9-Cbz |
| 154399 | 1,3-di-t-BuPh | 1,3-di-t-BuPh | 2-DBT | 9-Cbz | 9-Cbz |
| 154400 | 1,3-di-t-BuPh | 1,3-di-t-BuPh | 4-t-BuPh | 9-Cbz | 9-Cbz |
| 154401 | 1,3-di-t-BuPh | 1,3-di-t-BuPh | 1,3-di-t-BuPh | 9-Cbz | 9-Cbz |
| 154402 | 1,3-di-t-BuPh | 1,3-di-t-BuPh | 9-Cbz | 9-Cbz | 9-Cbz |
| 154403 | 1,3-di-t-BuPh | 1,3-di-t-BuPh | t-Bu | 4-t-BuPh | 4-t-BuPh |
| 154404 | 1,3-di-t-BuPh | 1,3-di-t-BuPh | Ph | 4-t-BuPh | 4-t-BuPh |
| 154405 | 1,3-di-t-BuPh | 1,3-di-t-BuPh | CN | 4-t-BuPh | 4-t-BuPh |
| 154406 | 1,3-di-t-BuPh | 1,3-di-t-BuPh | DPA | 4-t-BuPh | 4-t-BuPh |
| 154407 | 1,3-di-t-BuPh | 1,3-di-t-BuPh | 2-DBF | 4-t-BuPh | 4-t-BuPh |
| 154408 | 1,3-di-t-BuPh | 1,3-di-t-BuPh | 2-DBT | 4-t-BuPh | 4-t-BuPh |
| 154409 | 1,3-di-t-BuPh | 1,3-di-t-BuPh | 4-t-BuPh | 4-t-BuPh | 4-t-BuPh |
| 154410 | 1,3-di-t-BuPh | 1,3-di-t-BuPh | 1,3-di-t-BuPh | 4-t-BuPh | 4-t-BuPh |
| 154411 | 1,3-di-t-BuPh | 1,3-di-t-BuPh | 9-Cbz | 4-t-BuPh | 4-t-BuPh |
| 154412 | 1,3-di-t-BuPh | 1,3-di-t-BuPh | t-Bu | 1,3-di-t-BuPh | 1,3-di-t-BuPh |
| 154413 | 1,3-di-t-BuPh | 1,3-di-t-BuPh | Ph | 1,3-di-t-BuPh | 1,3-di-t-BuPh |
| 154414 | 1,3-di-t-BuPh | 1,3-di-t-BuPh | CN | 1,3-di-t-BuPh | 1,3-di-t-BuPh |
| 154415 | 1,3-di-t-BuPh | 1,3-di-t-BuPh | DPA | 1,3-di-t-BuPh | 1,3-di-t-BuPh |
| 154416 | 1,3-di-t-BuPh | 1,3-di-t-BuPh | 2-DBF | 1,3-di-t-BuPh | 1,3-di-t-BuPh |
| 154417 | 1,3-di-t-BuPh | 1,3-di-t-BuPh | 2-DBT | 1,3-di-t-BuPh | 1,3-di-t-BuPh |
| 154418 | 1,3-di-t-BuPh | 1,3-di-t-BuPh | 4-t-BuPh | 1,3-di-t-BuPh | 1,3-di-t-BuPh |
| 154419 | 1,3-di-t-BuPh | 1,3-di-t-BuPh | 1,3-di-t-BuPh | 1,3-di-t-BuPh | 1,3-di-t-BuPh |
| 154420 | 1,3-di-t-BuPh | 1,3-di-t-BuPh | 9-Cbz | 1,3-di-t-BuPh | 1,3-di-t-BuPh |

### Group HC9

The heterocyclic compound may be represented by Formula 16-4, and substituents may be defined as shown in Table 9.

TABLE 9

| Compound No. | R_{11b} | R_{12c} | R_{15C} | R_{18b} | R_{19b} |
|---|---|---|---|---|---|
| 164001 | t-Bu | t-Bu | t-Bu | t-Bu | t-Bu |
| 164002 | Ph | Ph | t-Bu | Ph | Ph |
| 164003 | t-Bu | t-Bu | H | H | H |
| 164004 | H | H | t-Bu | H | H |
| 164005 | H | H | H | t-Bu | t-Bu |
| 164006 | t-Bu | t-Bu | t-Bu | H | H |
| 164007 | H | H | t-Bu | t-Bu | t-Bu |
| 164008 | t-Bu | t-Bu | H | t-Bu | t-Bu |
| 164009 | Ph | Ph | H | H | H |
| 164010 | H | H | Ph | H | H |
| 164011 | H | H | H | Ph | Ph |
| 164012 | Ph | Ph | Ph | H | H |
| 164013 | H | H | Ph | Ph | Ph |
| 164014 | Ph | Ph | H | Ph | Ph |
| 164015 | CN | CN | H | H | H |
| 164016 | H | H | CN | H | H |
| 164017 | H | H | H | CN | CN |
| 164018 | CN | CN | CN | H | H |
| 164019 | H | H | CN | CN | CN |
| 164020 | CN | CN | H | CN | CN |
| 164021 | 9-Cbz | 9-Cbz | H | H | H |
| 164022 | H | H | 9-Cbz | H | H |
| 164023 | H | H | H | 9-Cbz | 9-Cbz |
| 164024 | 9-Cbz | 9-Cbz | 9-Cbz | H | H |
| 164025 | H | H | 9-Cbz | 9-Cbz | 9-Cbz |
| 164026 | 9-Cbz | 9-Cbz | H | 9-Cbz | 9-Cbz |
| 164027 | Me | Me | H | H | H |
| 164028 | H | H | Me | H | H |
| 164029 | H | H | H | Me | Me |
| 164030 | Me | Me | Me | H | H |
| 164031 | H | H | Me | Me | Me |
| 164032 | Me | Me | H | Me | Me |
| 164033 | CD₃ | CD3 | H | H | H |
| 164034 | H | H | CD3 | H | H |
| 164035 | H | H | H | CD3 | CD3 |
| 164036 | CD3 | CD3 | CD3 | H | H |
| 164037 | H | H | CD3 | CD3 | CD3 |
| 164038 | CD3 | CD3 | H | CD3 | CD3 |
| 164039 | 4-t-BuPh | 4-t-BuPh | H | H | H |
| 164040 | H | H | 4-t-BuPh | H | H |
| 164041 | H | H | H | 4-t-BuPh | 4-t-BuPh |
| 164042 | 4-t-BuPh | 4-t-BuPh | 4-t-BuPh | H | H |
| 164043 | H | H | 4-t-BuPh | 4-t-BuPh | 4-t-BuPh |
| 164044 | 4-t-BuPh | 4-t-BuPh | H | 4-t-BuPh | 4-t-BuPh |
| 164045 | 1,3-di-t-BuPh | 1,3-di-t-BuPh | H | H | H |
| 164046 | H | H | 1,3-di-t-BuPh | H | H |
| 164047 | H | H | H | 1,3-di-t-BuPh | 1,3-di-t-BuPh |
| 164048 | 1,3-di-t-BuPh | 1,3-di-t-BuPh | 1,3-di-t-BuPh | H | H |
| 164049 | H | H | 1,3-di-t-BuPh | 1,3-di-t-BuPh | 1,3-di-t-BuPh |
| 164050 | 1,3-di-t-BuPh | 1,3-di-t-BuPh | H | 1,3-di-t-BuPh | 1,3-di-t-BuPh |
| 164051 | t-Bu | t-Bu | Ph | H | H |
| 164052 | t-Bu | t-Bu | CN | H | H |
| 164053 | t-Bu | t-Bu | DPA | H | H |
| 164054 | t-Bu | t-Bu | 2-DBF | H | H |
| 164055 | t-Bu | t-Bu | 2-DBT | H | H |
| 164056 | t-Bu | t-Bu | 4-t-BuPh | H | H |
| 164057 | t-Bu | t-Bu | 1,3-di-t-BuPh | H | H |
| 164058 | t-Bu | t-Bu | 9-Cbz | H | H |
| 164059 | t-Bu | t-Bu | Ph | t-Bu | t-Bu |
| 164060 | t-Bu | t-Bu | CN | t-Bu | t-Bu |
| 164061 | t-Bu | t-Bu | DPA | t-Bu | t-Bu |
| 164062 | t-Bu | t-Bu | 2-DBF | t-Bu | t-Bu |
| 164063 | t-Bu | t-Bu | 2-DBT | t-Bu | t-Bu |
| 164064 | t-Bu | t-Bu | 4-t-BuPh | t-Bu | t-Bu |
| 164065 | t-Bu | t-Bu | 1,3-di-t-BuPh | t-Bu | t-Bu |
| 164066 | t-Bu | t-Bu | 9-Cbz | t-Bu | t-Bu |
| 164067 | t-Bu | t-Bu | t-Bu | Ph | Ph |
| 164068 | t-Bu | t-Bu | Ph | Ph | Ph |
| 164069 | t-Bu | t-Bu | CN | Ph | Ph |
| 164070 | t-Bu | t-Bu | DPA | Ph | Ph |
| 164071 | t-Bu | t-Bu | 2-DBF | Ph | Ph |
| 164072 | t-Bu | t-Bu | 2-DBT | Ph | Ph |
| 164073 | t-Bu | t-Bu | 4-t-BuPh | Ph | Ph |
| 164074 | t-Bu | t-Bu | 1,3-di-t-BuPh | Ph | Ph |
| 164075 | t-Bu | t-Bu | 9-Cbz | Ph | Ph |
| 164076 | t-Bu | t-Bu | t-Bu | CN | CN |
| 164077 | t-Bu | t-Bu | Ph | CN | CN |
| 164078 | t-Bu | t-Bu | CN | CN | CN |
| 164079 | t-Bu | t-Bu | DPA | CN | CN |
| 164080 | t-Bu | t-Bu | 2-DBF | CN | CN |
| 164081 | t-Bu | t-Bu | 2-DBT | CN | CN |
| 164082 | t-Bu | t-Bu | 4-t-BuPh | CN | CN |
| 164083 | t-Bu | t-Bu | 1,3-di-t-BuPh | CN | CN |
| 164084 | t-Bu | t-Bu | 9-Cbz | CN | CN |
| 164085 | t-Bu | t-Bu | t-Bu | 9-Cbz | 9-Cbz |
| 164086 | t-Bu | t-Bu | Ph | 9-Cbz | 9-Cbz |
| 164087 | t-Bu | t-Bu | CN | 9-Cbz | 9-Cbz |
| 164088 | t-Bu | t-Bu | DPA | 9-Cbz | 9-Cbz |
| 164089 | t-Bu | t-Bu | 2-DBF | 9-Cbz | 9-Cbz |
| 164090 | t-Bu | t-Bu | 2-DBT | 9-Cbz | 9-Cbz |
| 164091 | t-Bu | t-Bu | 4-t-BuPh | 9-Cbz | 9-Cbz |
| 164092 | t-Bu | t-Bu | 1,3-di-t-BuPh | 9-Cbz | 9-Cbz |
| 164093 | t-Bu | t-Bu | 9-Cbz | 9-Cbz | 9-Cbz |
| 164094 | t-Bu | t-Bu | t-Bu | 4-t-BuPh | 4-t-BuPh |
| 164095 | t-Bu | t-Bu | Ph | 4-t-BuPh | 4-t-BuPh |
| 164096 | t-Bu | t-Bu | CN | 4-t-BuPh | 4-t-BuPh |
| 164097 | t-Bu | t-Bu | DPA | 4-t-BuPh | 4-t-BuPh |
| 164098 | t-Bu | t-Bu | 2-DBF | 4-t-BuPh | 4-t-BuPh |
| 164099 | t-Bu | t-Bu | 2-DBT | 4-t-BuPh | 4-t-BuPh |
| 164100 | t-Bu | t-Bu | 4-t-BuPh | 4-t-BuPh | 4-t-BuPh |
| 164101 | t-Bu | t-Bu | 1,3-di-t-BuPh | 4-t-BuPh | 4-t-BuPh |
| 164102 | t-Bu | t-Bu | 9-Cbz | 4-t-BuPh | 4-t-BuPh |
| 164103 | t-Bu | t-Bu | t-Bu | 1,3-di-t-BuPh | 1,3-di-t-BuPh |
| 164104 | t-Bu | t-Bu | Ph | 1,3-di-t-BuPh | 1,3-di-t-BuPh |
| 164105 | t-Bu | t-Bu | CN | 1,3-di-t-BuPh | 1,3-di-t-BuPh |
| 164106 | t-Bu | t-Bu | DPA | 1,3-di-t-BuPh | 1,3-di-t-BuPh |
| 164107 | t-Bu | t-Bu | 2-DBF | 1,3-di-t-BuPh | 1,3-di-t-BuPh |
| 164108 | t-Bu | t-Bu | 2-DBT | 1,3-di-t-BuPh | 1,3-di-t-BuPh |
| 164109 | t-Bu | t-Bu | 4-t-BuPh | 1,3-di-t-BuPh | 1,3-di-t-BuPh |
| 164110 | t-Bu | t-Bu | 1,3-di-t-BuPh | 1,3-di-t-BuPh | 1,3-di-t-BuPh |
| 164111 | t-Bu | t-Bu | 9-Cbz | 1,3-di-t-BuPh | 1,3-di-t-BuPh |
| 164112 | Ph | Ph | t-Bu | H | H |
| 164113 | Ph | Ph | CN | H | H |
| 164114 | Ph | Ph | DPA | H | H |
| 164115 | Ph | Ph | 2-DBF | H | H |
| 164116 | Ph | Ph | 2-DBT | H | H |
| 164117 | Ph | Ph | 4-t-BuPh | H | H |
| 164118 | Ph | Ph | 1,3-di-t-BuPh | H | H |
| 164119 | Ph | Ph | 9-Cbz | H | H |
| 164120 | Ph | Ph | t-Bu | t-Bu | t-Bu |
| 164121 | Ph | Ph | Ph | t-Bu | t-Bu |
| 164122 | Ph | Ph | CN | t-Bu | t-Bu |
| 164123 | Ph | Ph | DPA | t-Bu | t-Bu |
| 164124 | Ph | Ph | 2-DBF | t-Bu | t-Bu |
| 164125 | Ph | Ph | 2-DBT | t-Bu | t-Bu |
| 164126 | Ph | Ph | 4-t-BuPh | t-Bu | t-Bu |
| 164127 | Ph | Ph | 1,3-di-t-BuPh | t-Bu | t-Bu |
| 164128 | Ph | Ph | 9-Cbz | t-Bu | t-Bu |
| 164129 | Ph | Ph | Ph | Ph | Ph |
| 164130 | Ph | Ph | CN | Ph | Ph |
| 164131 | Ph | Ph | DPA | Ph | Ph |
| 164132 | Ph | Ph | 2-DBF | Ph | Ph |
| 164133 | Ph | Ph | 2-DBT | Ph | Ph |
| 164134 | Ph | Ph | 4-t-BuPh | Ph | Ph |
| 164135 | Ph | Ph | 1,3-di-t-BuPh | Ph | Ph |
| 164136 | Ph | Ph | 9-Cbz | Ph | Ph |
| 164137 | Ph | Ph | t-Bu | CN | CN |
| 164138 | Ph | Ph | Ph | CN | CN |
| 164139 | Ph | Ph | CN | CN | CN |
| 164140 | Ph | Ph | DPA | CN | CN |
| 164141 | Ph | Ph | 2-DBF | CN | CN |
| 164142 | Ph | Ph | 2-DBT | CN | CN |
| 164143 | Ph | Ph | 4-t-BuPh | CN | CN |
| 164144 | Ph | Ph | 1,3-di-t-BuPh | CN | CN |
| 164145 | Ph | Ph | 9-Cbz | CN | CN |
| 164146 | Ph | Ph | t-Bu | 9-Cbz | 9-Cbz |
| 164147 | Ph | Ph | Ph | 9-Cbz | 9-Cbz |
| 164148 | Ph | Ph | CN | 9-Cbz | 9-Cbz |
| 164149 | Ph | Ph | DPA | 9-Cbz | 9-Cbz |
| 164150 | Ph | Ph | 2-DBF | 9-Cbz | 9-Cbz |
| 164151 | Ph | Ph | 2-DBT | 9-Cbz | 9-Cbz |
| 164152 | Ph | Ph | 4-t-BuPh | 9-Cbz | 9-Cbz |
| 164153 | Ph | Ph | 1,3-di-t-BuPh | 9-Cbz | 9-Cbz |
| 164154 | Ph | Ph | 9-Cbz | 9-Cbz | 9-Cbz |
| 164155 | Ph | Ph | t-Bu | 4-t-BuPh | 4-t-BuPh |
| 164156 | Ph | Ph | Ph | 4-t-BuPh | 4-t-BuPh |
| 164157 | Ph | Ph | CN | 4-t-BuPh | 4-t-BuPh |
| 164158 | Ph | Ph | DPA | 4-t-BuPh | 4-t-BuPh |
| 164159 | Ph | Ph | 2-DBF | 4-t-BuPh | 4-t-BuPh |
| 164160 | Ph | Ph | 2-DBT | 4-t-BuPh | 4-t-BuPh |
| 164161 | Ph | Ph | 4-t-BuPh | 4-t-BuPh | 4-t-BuPh |
| 164162 | Ph | Ph | 1,3-di-t-BuPh | 4-t-BuPh | 4-t-BuPh |
| 164163 | Ph | Ph | 9-Cbz | 4-t-BuPh | 4-t-BuPh |
| 164164 | Ph | Ph | t-Bu | 1,3-di-t-BuPh | 1,3-di-t-BuPh |
| 164165 | Ph | Ph | Ph | 1,3-di-t-BuPh | 1,3-di-t-BuPh |
| 164166 | Ph | Ph | CN | 1,3-di-t-BuPh | 1,3-di-t-BuPh |
| 164167 | Ph | Ph | DPA | 1,3-di-t-BuPh | 1,3-di-t-BuPh |
| 164168 | Ph | Ph | 2-DBF | 1,3-di-t-BuPh | 1,3-di-t-BuPh |
| 164169 | Ph | Ph | 2-DBT | 1,3-di-t-BuPh | 1,3-di-t-BuPh |
| 164170 | Ph | Ph | 4-t-BuPh | 1,3-di-t-BuPh | 1,3-di-t-BuPh |
| 164171 | Ph | Ph | 1,3-di-t-BuPh | 1,3-di-t-BuPh | 1,3-di-t-BuPh |
| 164172 | Ph | Ph | 9-Cbz | 1,3-di-t-BuPh | 1,3-di-t-BuPh |
| 164173 | 9-Cbz | 9-Cbz | t-Bu | H | H |
| 164174 | 9-Cbz | 9-Cbz | Ph | H | H |
| 164175 | 9-Cbz | 9-Cbz | CN | H | H |
| 164176 | 9-Cbz | 9-Cbz | DPA | H | H |
| 164177 | 9-Cbz | 9-Cbz | 2-DBF | H | H |
| 164178 | 9-Cbz | 9-Cbz | 2-DBT | H | H |
| 164179 | 9-Cbz | 9-Cbz | 4-t-BuPh | H | H |
| 164180 | 9-Cbz | 9-Cbz | 1,3-di-t-BuPh | H | H |
| 164181 | 9-Cbz | 9-Cbz | t-Bu | t-Bu | t-Bu |
| 164182 | 9-Cbz | 9-Cbz | Ph | t-Bu | t-Bu |
| 164183 | 9-Cbz | 9-Cbz | CN | t-Bu | t-Bu |
| 164184 | 9-Cbz | 9-Cbz | DPA | t-Bu | t-Bu |
| 164185 | 9-Cbz | 9-Cbz | 2-DBF | t-Bu | t-Bu |
| 164186 | 9-Cbz | 9-Cbz | 2-DBT | t-Bu | t-Bu |
| 164187 | 9-Cbz | 9-Cbz | 4-t-BuPh | t-Bu | t-Bu |
| 164188 | 9-Cbz | 9-Cbz | 1,3-di-t-BuPh | t-Bu | t-Bu |
| 164189 | 9-Cbz | 9-Cbz | 9-Cbz | t-Bu | t-Bu |
| 164190 | 9-Cbz | 9-Cbz | t-Bu | Ph | Ph |
| 164191 | 9-Cbz | 9-Cbz | Ph | Ph | Ph |
| 164192 | 9-Cbz | 9-Cbz | CN | Ph | Ph |
| 164193 | 9-Cbz | 9-Cbz | DPA | Ph | Ph |
| 164194 | 9-Cbz | 9-Cbz | 2-DBF | Ph | Ph |
| 164195 | 9-Cbz | 9-Cbz | 2-DBT | Ph | Ph |
| 164196 | 9-Cbz | 9-Cbz | 4-t-BuPh | Ph | Ph |
| 164197 | 9-Cbz | 9-Cbz | 1,3-di-t-BuPh | Ph | Ph |
| 164198 | 9-Cbz | 9-Cbz | 9-Cbz | Ph | Ph |
| 164199 | 9-Cbz | 9-Cbz | t-Bu | CN | CN |
| 164200 | 9-Cbz | 9-Cbz | Ph | CN | CN |
| 164201 | 9-Cbz | 9-Cbz | CN | CN | CN |
| 164202 | 9-Cbz | 9-Cbz | DPA | CN | CN |
| 164203 | 9-Cbz | 9-Cbz | 2-DBF | CN | CN |
| 164204 | 9-Cbz | 9-Cbz | 2-DBT | CN | CN |
| 164205 | 9-Cbz | 9-Cbz | 4-t-BuPh | CN | CN |
| 164206 | 9-Cbz | 9-Cbz | 1,3-di-t-BuPh | CN | CN |
| 164207 | 9-Cbz | 9-Cbz | 9-Cbz | CN | CN |
| 164208 | 9-Cbz | 9-Cbz | t-Bu | 9-Cbz | 9-Cbz |
| 164209 | 9-Cbz | 9-Cbz | Ph | 9-Cbz | 9-Cbz |
| 164210 | 9-Cbz | 9-Cbz | CN | 9-Cbz | 9-Cbz |
| 164211 | 9-Cbz | 9-Cbz | DPA | 9-Cbz | 9-Cbz |
| 164212 | 9-Cbz | 9-Cbz | 2-DBF | 9-Cbz | 9-Cbz |
| 164213 | 9-Cbz | 9-Cbz | 2-DBT | 9-Cbz | 9-Cbz |
| 164214 | 9-Cbz | 9-Cbz | 4-t-BuPh | 9-Cbz | 9-Cbz |
| 164215 | 9-Cbz | 9-Cbz | 1,3-di-t-BuPh | 9-Cbz | 9-Cbz |
| 164216 | 9-Cbz | 9-Cbz | 9-Cbz | 9-Cbz | 9-Cbz |
| 164217 | 9-Cbz | 9-Cbz | t-Bu | 4-t-BuPh | 4-t-BuPh |
| 164218 | 9-Cbz | 9-Cbz | Ph | 4-t-BuPh | 4-t-BuPh |
| 164219 | 9-Cbz | 9-Cbz | CN | 4-t-BuPh | 4-t-BuPh |
| 164220 | 9-Cbz | 9-Cbz | DPA | 4-t-BuPh | 4-t-BuPh |
| 164221 | 9-Cbz | 9-Cbz | 2-DBF | 4-t-BuPh | 4-t-BuPh |
| 164222 | 9-Cbz | 9-Cbz | 2-DBT | 4-t-BuPh | 4-t-BuPh |
| 164223 | 9-Cbz | 9-Cbz | 4-t-BuPh | 4-t-BuPh | 4-t-BuPh |
| 164224 | 9-Cbz | 9-Cbz | 1,3-di-t-BuPh | 4-t-BuPh | 4-t-BuPh |
| 164225 | 9-Cbz | 9-Cbz | 9-Cbz | 4-t-BuPh | 4-t-BuPh |
| 164226 | 9-Cbz | 9-Cbz | t-Bu | 1,3-di-t-BuPh | 1,3-di-t-BuPh |
| 164227 | 9-Cbz | 9-Cbz | Ph | 1,3-di-t-BuPh | 1,3-di-t-BuPh |
| 164228 | 9-Cbz | 9-Cbz | CN | 1,3-di-t-BuPh | 1,3-di-t-BuPh |
| 164229 | 9-Cbz | 9-Cbz | DPA | 1,3-di-t-BuPh | 1,3-di-t-BuPh |
| 164230 | 9-Cbz | 9-Cbz | 2-DBF | 1,3-di-t-BuPh | 1,3-di-t-BuPh |
| 164231 | 9-Cbz | 9-Cbz | 2-DBT | 1,3-di-t-BuPh | 1,3-di-t-BuPh |
| 164232 | 9-Cbz | 9-Cbz | 4-t-BuPh | 1,3-di-t-BuPh | 1,3-di-t-BuPh |
| 164233 | 9-Cbz | 9-Cbz | 1,3-di-t-BuPh | 1,3-di-t-BuPh | 1,3-di-t-BuPh |
| 164234 | 9-Cbz | 9-Cbz | 9-Cbz | 1,3-di-t-BuPh | 1,3-di-t-BuPh |
| 164235 | CN | CN | t-Bu | H | H |
| 164236 | CN | CN | Ph | H | H |
| 164237 | CN | CN | DPA | H | H |
| 164238 | CN | CN | 2-DBF | H | H |
| 164239 | CN | CN | 2-DBT | H | H |
| 164240 | CN | CN | 4-t-BuPh | H | H |
| 164241 | CN | CN | 1,3-di-t-BuPh | H | H |
| 164242 | CN | CN | 9-Cbz | H | H |
| 164243 | CN | CN | t-Bu | t-Bu | t-Bu |
| 164244 | CN | CN | Ph | t-Bu | t-Bu |
| 164245 | CN | CN | CN | t-Bu | t-Bu |
| 164246 | CN | CN | DPA | t-Bu | t-Bu |
| 164247 | CN | CN | 2-DBF | t-Bu | t-Bu |
| 164248 | CN | CN | 2-DBT | t-Bu | t-Bu |
| 164249 | CN | CN | 4-t-BuPh | t-Bu | t-Bu |
| 164250 | CN | CN | 1,3-di-t-BuPh | t-Bu | t-Bu |
| 164251 | CN | CN | 9-Cbz | t-Bu | t-Bu |
| 164252 | CN | CN | t-Bu | Ph | Ph |
| 164253 | CN | CN | Ph | Ph | Ph |
| 164254 | CN | CN | CN | Ph | Ph |
| 164255 | CN | CN | DPA | Ph | Ph |
| 164256 | CN | CN | 2-DBF | Ph | Ph |
| 164257 | CN | CN | 2-DBT | Ph | Ph |
| 164258 | CN | CN | 4-t-BuPh | Ph | Ph |
| 164259 | CN | CN | 1,3-di-t-BuPh | Ph | Ph |
| 164260 | CN | CN | 9-Cbz | Ph | Ph |
| 164261 | CN | CN | t-Bu | CN | CN |
| 164262 | CN | CN | Ph | CN | CN |
| 164263 | CN | CN | CN | CN | CN |
| 164264 | CN | CN | DPA | CN | CN |
| 164265 | CN | CN | 2-DBF | CN | CN |
| 164266 | CN | CN | 2-DBT | CN | CN |
| 164267 | CN | CN | 4-t-BuPh | CN | CN |
| 164268 | CN | CN | 1,3-di-t-BuPh | CN | CN |
| 164269 | CN | CN | 9-Cbz | CN | CN |
| 164270 | CN | CN | t-Bu | 9-Cbz | 9-Cbz |
| 164271 | CN | CN | Ph | 9-Cbz | 9-Cbz |
| 164272 | CN | CN | CN | 9-Cbz | 9-Cbz |
| 164273 | CN | CN | DPA | 9-Cbz | 9-Cbz |
| 164274 | CN | CN | 2-DBF | 9-Cbz | 9-Cbz |
| 164275 | CN | CN | 2-DBT | 9-Cbz | 9-Cbz |
| 164276 | CN | CN | 4-t-BuPh | 9-Cbz | 9-Cbz |
| 164277 | CN | CN | 1,3-di-t-BuPh | 9-Cbz | 9-Cbz |
| 164278 | CN | CN | 9-Cbz | 9-Cbz | 9-Cbz |
| 164279 | CN | CN | t-Bu | 4-t-BuPh | 4-t-BuPh |
| 164280 | CN | CN | Ph | 4-t-BuPh | 4-t-BuPh |
| 164281 | CN | CN | CN | 4-t-BuPh | 4-t-BuPh |
| 164282 | CN | CN | DPA | 4-t-BuPh | 4-t-BuPh |
| 164283 | CN | CN | 2-DBF | 4-t-BuPh | 4-t-BuPh |
| 164284 | CN | CN | 2-DBT | 4-t-BuPh | 4-t-BuPh |
| 164285 | CN | CN | 4-t-BuPh | 4-t-BuPh | 4-t-BuPh |
| 164286 | CN | CN | 1,3-di-t-BuPh | 4-t-BuPh | 4-t-BuPh |
| 164287 | CN | CN | 9-Cbz | 4-t-BuPh | 4-t-BuPh |
| 164288 | CN | CN | t-Bu | 1,3-di-t-BuPh | 1,3-di-t-BuPh |
| 164289 | CN | CN | Ph | 1,3-di-t-BuPh | 1,3-di-t-BuPh |
| 164290 | CN | CN | CN | 1,3-di-t-BuPh | 1,3-di-t-BuPh |
| 164291 | CN | CN | DPA | 1,3-di-t-BuPh | 1,3-di-t-BuPh |
| 164292 | CN | CN | 2-DBF | 1,3-di-t-BuPh | 1,3-di-t-BuPh |
| 164293 | CN | CN | 2-DBT | 1,3-di-t-BuPh | 1,3-di-t-BuPh |
| 164294 | CN | CN | 4-t-BuPh | 1,3-di-t-BuPh | 1,3-di-t-BuPh |
| 164295 | CN | CN | 1,3-di-t-BuPh | 1,3-di-t-BuPh | 1,3-di-t-BuPh |
| 164296 | CN | CN | 9-Cbz | 1,3-di-t-BuPh | 1,3-di-t-BuPh |
| 164297 | 4-t-BuPh | 4-t-BuPh | t-Bu | H | H |
| 164298 | 4-t-BuPh | 4-t-BuPh | Ph | H | H |
| 164299 | 4-t-BuPh | 4-t-BuPh | CN | H | H |
| 164300 | 4-t-BuPh | 4-t-BuPh | DPA | H | H |
| 164301 | 4-t-BuPh | 4-t-BuPh | 2-DBF | H | H |
| 164302 | 4-t-BuPh | 4-t-BuPh | 2-DBT | H | H |
| 164303 | 4-t-BuPh | 4-t-BuPh | 1,3-di-t-BuPh | H | H |
| 164304 | 4-t-BuPh | 4-t-BuPh | 9-Cbz | H | H |
| 164305 | 4-t-BuPh | 4-t-BuPh | t-Bu | t-Bu | t-Bu |
| 164306 | 4-t-BuPh | 4-t-BuPh | Ph | t-Bu | t-Bu |
| 164307 | 4-t-BuPh | 4-t-BuPh | CN | t-Bu | t-Bu |
| 164308 | 4-t-BuPh | 4-t-BuPh | DPA | t-Bu | t-Bu |
| 164309 | 4-t-BuPh | 4-t-BuPh | 2-DBF | t-Bu | t-Bu |
| 164310 | 4-t-BuPh | 4-t-BuPh | 2-DBT | t-Bu | t-Bu |
| 164311 | 4-t-BuPh | 4-t-BuPh | 4-t-BuPh | t-Bu | t-Bu |
| 164312 | 4-t-BuPh | 4-t-BuPh | 1,3-di-t-BuPh | t-Bu | t-Bu |
| 164313 | 4-t-BuPh | 4-t-BuPh | 9-Cbz | t-Bu | t-Bu |
| 164314 | 4-t-BuPh | 4-t-BuPh | t-Bu | Ph | Ph |
| 164315 | 4-t-BuPh | 4-t-BuPh | Ph | Ph | Ph |
| 164316 | 4-t-BuPh | 4-t-BuPh | CN | Ph | Ph |
| 164317 | 4-t-BuPh | 4-t-BuPh | DPA | Ph | Ph |
| 164318 | 4-t-BuPh | 4-t-BuPh | 2-DBF | Ph | Ph |
| 164319 | 4-t-BuPh | 4-t-BuPh | 2-DBT | Ph | Ph |
| 164320 | 4-t-BuPh | 4-t-BuPh | 4-t-BuPh | Ph | Ph |
| 164321 | 4-t-BuPh | 4-t-BuPh | 1,3-di-t-BuPh | Ph | Ph |
| 164322 | 4-t-BuPh | 4-t-BuPh | 9-Cbz | Ph | Ph |
| 164323 | 4-t-BuPh | 4-t-BuPh | t-Bu | CN | CN |
| 164324 | 4-t-BuPh | 4-t-BuPh | Ph | CN | CN |
| 164325 | 4-t-BuPh | 4-t-BuPh | CN | CN | CN |
| 164326 | 4-t-BuPh | 4-t-BuPh | DPA | CN | CN |
| 164327 | 4-t-BuPh | 4-t-BuPh | 2-DBF | CN | CN |
| 164328 | 4-t-BuPh | 4-t-BuPh | 2-DBT | CN | CN |
| 164329 | 4-t-BuPh | 4-t-BuPh | 4-t-BuPh | CN | CN |
| 164330 | 4-t-BuPh | 4-t-BuPh | 1,3-di-t-BuPh | CN | CN |
| 164331 | 4-t-BuPh | 4-t-BuPh | 9-Cbz | CN | CN |
| 164332 | 4-t-BuPh | 4-t-BuPh | t-Bu | 9-Cbz | 9-Cbz |
| 164333 | 4-t-BuPh | 4-t-BuPh | Ph | 9-Cbz | 9-Cbz |
| 164334 | 4-t-BuPh | 4-t-BuPh | CN | 9-Cbz | 9-Cbz |
| 164335 | 4-t-BuPh | 4-t-BuPh | DPA | 9-Cbz | 9-Cbz |
| 164336 | 4-t-BuPh | 4-t-BuPh | 2-DBF | 9-Cbz | 9-Cbz |
| 164337 | 4-t-BuPh | 4-t-BuPh | 2-DBT | 9-Cbz | 9-Cbz |
| 164338 | 4-t-BuPh | 4-t-BuPh | 4-t-BuPh | 9-Cbz | 9-Cbz |
| 164339 | 4-t-BuPh | 4-t-BuPh | 1,3-di-t-BuPh | 9-Cbz | 9-Cbz |
| 164340 | 4-t-BuPh | 4-t-BuPh | 9-Cbz | 9-Cbz | 9-Cbz |
| 164341 | 4-t-BuPh | 4-t-BuPh | t-Bu | 4-t-BuPh | 4-t-BuPh |
| 164342 | 4-t-BuPh | 4-t-BuPh | Ph | 4-t-BuPh | 4-t-BuPh |
| 164343 | 4-t-BuPh | 4-t-BuPh | CN | 4-t-BuPh | 4-t-BuPh |
| 164344 | 4-t-BuPh | 4-t-BuPh | DPA | 4-t-BuPh | 4-t-BuPh |
| 164345 | 4-t-BuPh | 4-t-BuPh | 2-DBF | 4-t-BuPh | 4-t-BuPh |
| 164346 | 4-t-BuPh | 4-t-BuPh | 2-DBT | 4-t-BuPh | 4-t-BuPh |
| 164347 | 4-t-BuPh | 4-t-BuPh | 4-t-BuPh | 4-t-BuPh | 4-t-BuPh |
| 164348 | 4-t-BuPh | 4-t-BuPh | 1,3-di-t-BuPh | 4-t-BuPh | 4-t-BuPh |
| 164349 | 4-t-BuPh | 4-t-BuPh | 9-Cbz | 4-t-BuPh | 4-t-BuPh |
| 164350 | 4-t-BuPh | 4-t-BuPh | t-Bu | 1,3-di-t-BuPh | 1,3-di-t-BuPh |
| 164351 | 4-t-BuPh | 4-t-BuPh | Ph | 1,3-di-t-BuPh | 1,3-di-t-BuPh |
| 164352 | 4-t-BuPh | 4-t-BuPh | CN | 1,3-di-t-BuPh | 1,3-di-t-BuPh |
| 164353 | 4-t-BuPh | 4-t-BuPh | DPA | 1,3-di-t-BuPh | 1,3-di-t-BuPh |
| 164354 | 4-t-BuPh | 4-t-BuPh | 2-DBF | 1,3-di-t-BuPh | 1,3-di-t-BuPh |
| 164355 | 4-t-BuPh | 4-t-BuPh | 2-DBT | 1,3-di-t-BuPh | 1,3-di-t-BuPh |
| 164356 | 4-t-BuPh | 4-t-BuPh | 4-t-BuPh | 1,3-di-t-BuPh | 1,3-di-t-BuPh |
| 164357 | 4-t-BuPh | 4-t-BuPh | 1,3-di-t-BuPh | 1,3-di-t-BuPh | 1,3-di-t-BuPh |
| 164358 | 4-t-BuPh | 4-t-BuPh | 9-Cbz | 1,3-di-t-BuPh | 1,3-di-t-BuPh |
| 164359 | 1,3-di-t-BuPh | 1,3-di-t-BuPh | t-Bu | H | H |
| 164360 | 1,3-di-t-BuPh | 1,3-di-t-BuPh | Ph | H | H |
| 164361 | 1,3-di-t-BuPh | 1,3-di-t-BuPh | CN | H | H |
| 164362 | 1,3-di-t-BuPh | 1,3-di-t-BuPh | DPA | H | H |
| 164363 | 1,3-di-t-BuPh | 1,3-di-t-BuPh | 2-DBF | H | H |
| 164364 | 1,3-di-t-BuPh | 1,3-di-t-BuPh | 2-DBT | H | H |
| 164365 | 1,3-di-t-BuPh | 1,3-di-t-BuPh | 4-t-BuPh | H | H |
| 164366 | 1,3-di-t-BuPh | 1,3-di-t-BuPh | 9-Cbz | H | H |
| 164367 | 1,3-di-t-BuPh | 1,3-di-t-BuPh | t-Bu | t-Bu | t-Bu |
| 164368 | 1,3-di-t-BuPh | 1,3-di-t-BuPh | Ph | t-Bu | t-Bu |
| 164369 | 1,3-di-t-BuPh | 1,3-di-t-BuPh | CN | t-Bu | t-Bu |
| 164370 | 1,3-di-t-BuPh | 1,3-di-t-BuPh | DPA | t-Bu | t-Bu |
| 164371 | 1,3-di-t-BuPh | 1,3-di-t-BuPh | 2-DBF | t-Bu | t-Bu |
| 164372 | 1,3-di-t-BuPh | 1,3-di-t-BuPh | 2-DBT | t-Bu | t-Bu |
| 164373 | 1,3-di-t-BuPh | 1,3-di-t-BuPh | 4-t-BuPh | t-Bu | t-Bu |
| 164374 | 1,3-di-t-BuPh | 1,3-di-t-BuPh | 1,3-di-t-BuPh | t-Bu | t-Bu |
| 164375 | 1,3-di-t-BuPh | 1,3-di-t-BuPh | 9-Cbz | t-Bu | t-Bu |
| 164376 | 1,3-di-t-BuPh | 1,3-di-t-BuPh | t-Bu | Ph | Ph |
| 164377 | 1,3-di-t-BuPh | 1,3-di-t-BuPh | Ph | Ph | Ph |
| 164378 | 1,3-di-t-BuPh | 1,3-di-t-BuPh | CN | Ph | Ph |
| 164379 | 1,3-di-t-BuPh | 1,3-di-t-BuPh | DPA | Ph | Ph |
| 164380 | 1,3-di-t-BuPh | 1,3-di-t-BuPh | 2-DBF | Ph | Ph |
| 164381 | 1,3-di-t-BuPh | 1,3-di-t-BuPh | 2-DBT | Ph | Ph |
| 164382 | 1,3-di-t-BuPh | 1,3-di-t-BuPh | 4-t-BuPh | Ph | Ph |
| 164383 | 1,3-di-t-BuPh | 1,3-di-t-BuPh | 1,3-di-t-BuPh | Ph | Ph |
| 164384 | 1,3-di-t-BuPh | 1,3-di-t-BuPh | 9-Cbz | Ph | Ph |
| 164385 | 1,3-di-t-BuPh | 1,3-di-t-BuPh | t-Bu | CN | CN |
| 164386 | 1,3-di-t-BuPh | 1,3-di-t-BuPh | Ph | CN | CN |
| 164387 | 1,3-di-t-BuPh | 1,3-di-t-BuPh | CN | CN | CN |
| 164388 | 1,3-di-t-BuPh | 1,3-di-t-BuPh | DPA | CN | CN |
| 164389 | 1,3-di-t-BuPh | 1,3-di-t-BuPh | 2-DBF | CN | CN |
| 164390 | 1,3-di-t-BuPh | 1,3-di-t-BuPh | 2-DBT | CN | CN |
| 164391 | 1,3-di-t-BuPh | 1,3-di-t-BuPh | 4-t-BuPh | CN | CN |
| 164392 | 1,3-di-t-BuPh | 1,3-di-t-BuPh | 1,3-di-t-BuPh | CN | CN |
| 164393 | 1,3-di-t-BuPh | 1,3-di-t-BuPh | 9-Cbz | CN | CN |
| 164394 | 1,3-di-t-BuPh | 1,3-di-t-BuPh | t-Bu | 9-Cbz | 9-Cbz |
| 164395 | 1,3-di-t-BuPh | 1,3-di-t-BuPh | Ph | 9-Cbz | 9-Cbz |
| 164396 | 1,3-di-t-BuPh | 1,3-di-t-BuPh | CN | 9-Cbz | 9-Cbz |
| 164397 | 1,3-di-t-BuPh | 1,3-di-t-BuPh | DPA | 9-Cbz | 9-Cbz |
| 164398 | 1,3-di-t-BuPh | 1,3-di-t-BuPh | 2-DBF | 9-Cbz | 9-Cbz |
| 164399 | 1,3-di-t-BuPh | 1,3-di-t-BuPh | 2-DBT | 9-Cbz | 9-Cbz |
| 164400 | 1,3-di-t-BuPh | 1,3-di-t-BuPh | 4-t-BuPh | 9-Cbz | 9-Cbz |
| 164401 | 1,3-di-t-BuPh | 1,3-di-t-BuPh | 1,3-di-t-BuPh | 9-Cbz | 9-Cbz |
| 164402 | 1,3-di-t-BuPh | 1,3-di-t-BuPh | 9-Cbz | 9-Cbz | 9-Cbz |
| 164403 | 1,3-di-t-BuPh | 1,3-di-t-BuPh | t-Bu | 4-t-BuPh | 4-t-BuPh |
| 164404 | 1,3-di-t-BuPh | 1,3-di-t-BuPh | Ph | 4-t-BuPh | 4-t-BuPh |
| 164405 | 1,3-di-t-BuPh | 1,3-di-t-BuPh | CN | 4-t-BuPh | 4-t-BuPh |
| 164406 | 1,3-di-t-BuPh | 1,3-di-t-BuPh | DPA | 4-t-BuPh | 4-t-BuPh |
| 164407 | 1,3-di-t-BuPh | 1,3-di-t-BuPh | 2-DBF | 4-t-BuPh | 4-t-BuPh |
| 164408 | 1,3-di-t-BuPh | 1,3-di-t-BuPh | 2-DBT | 4-t-BuPh | 4-t-BuPh |
| 164409 | 1,3-di-t-BuPh | 1,3-di-t-BuPh | 4-t-BuPh | 4-t-BuPh | 4-t-BuPh |
| 164410 | 1,3-di-t-BuPh | 1,3-di-t-BuPh | 1,3-di-t-BuPh | 4-t-BuPh | 4-t-BuPh |
| 164411 | 1,3-di-t-BuPh | 1,3-di-t-BuPh | 9-Cbz | 4-t-BuPh | 4-t-BuPh |
| 164412 | 1,3-di-t-BuPh | 1,3-di-t-BuPh | t-Bu | 1,3-di-t-BuPh | 1,3-di-t-BuPh |
| 164413 | 1,3-di-t-BuPh | 1,3-di-t-BuPh | Ph | 1,3-di-t-BuPh | 1,3-di-t-BuPh |
| 164414 | 1,3-di-t-BuPh | 1,3-di-t-BuPh | CN | 1,3-di-t-BuPh | 1,3-di-t-BuPh |
| 164415 | 1,3-di-t-BuPh | 1,3-di-t-BuPh | DPA | 1,3-di-t-BuPh | 1,3-di-t-BuPh |
| 164416 | 1,3-di-t-BuPh | 1,3-di-t-BuPh | 2-DBF | 1,3-di-t-BuPh | 1,3-di-t-BuPh |
| 164417 | 1,3-di-t-BuPh | 1,3-di-t-BuPh | 2-DBT | 1,3-di-t-BuPh | 1,3-di-t-BuPh |
| 164418 | 1,3-di-t-BuPh | 1,3-di-t-BuPh | 4-t-BuPh | 1,3-di-t-BuPh | 1,3-di-t-BuPh |
| 164419 | 1,3-di-t-BuPh | 1,3-di-t-BuPh | 1,3-di-t-BuPh | 1,3-di-t-BuPh | 1,3-di-t-BuPh |
| 164420 | 1,3-di-t-BuPh | 1,3-di-t-BuPh | 9-Cbz | 1,3-di-t-BuPh | 1,3-di-t-BuPh |

### Group HC10

The heterocyclic compound may be represented by Formula 17-4, and substituents may be defined as shown in Table 10.

TABLE 10

| Compound No. | R_{11b} | R_{12c} | R_{15C} | R_{18b} | R_{19b} |
|---|---|---|---|---|---|
| 174001 | t-Bu | t-Bu | t-Bu | t-Bu | t-Bu |
| 174002 | Ph | Ph | t-Bu | Ph | Ph |
| 174003 | t-Bu | t-Bu | H | H | H |
| 174004 | H | H | t-Bu | H | H |
| 174005 | H | H | H | t-Bu | t-Bu |
| 174006 | t-Bu | t-Bu | t-Bu | H | H |
| 174007 | H | H | t-Bu | t-Bu | t-Bu |
| 174008 | t-Bu | t-Bu | H | t-Bu | t-Bu |
| 174009 | Ph | Ph | H | H | H |
| 174010 | H | H | Ph | H | H |
| 174011 | H | H | H | Ph | Ph |
| 174012 | Ph | Ph | Ph | H | H |
| 174013 | H | H | Ph | Ph | Ph |
| 174014 | Ph | Ph | H | Ph | Ph |
| 174015 | CN | CN | H | H | H |
| 174016 | H | H | CN | H | H |
| 174017 | H | H | H | CN | CN |
| 174018 | CN | CN | CN | H | H |
| 174019 | H | H | CN | CN | CN |
| 174020 | CN | CN | H | CN | CN |
| 174021 | 9-Cbz | 9-Cbz | H | H | H |
| 174022 | H | H | 9-Cbz | H | H |
| 174023 | H | H | H | 9-Cbz | 9-Cbz |
| 174024 | 9-Cbz | 9-Cbz | 9-Cbz | H | H |
| 174025 | H | H | 9-Cbz | 9-Cbz | 9-Cbz |
| 174026 | 9-Cbz | 9-Cbz | H | 9-Cbz | 9-Cbz |
| 174027 | Me | Me | H | H | H |
| 174028 | H | H | Me | H | H |
| 174029 | H | H | H | Me | Me |
| 174030 | Me | Me | Me | H | H |
| 174031 | H | H | Me | Me | Me |
| 174032 | Me | Me | H | Me | Me |
| 174033 | CD₃ | CD3 | H | H | H |
| 174034 | H | H | CD3 | H | H |
| 174035 | H | H | H | CD3 | CD3 |
| 174036 | CD3 | CD3 | CD3 | H | H |
| 174037 | H | H | CD3 | CD3 | CD3 |
| 174038 | CD3 | CD3 | H | CD3 | CD3 |
| 174039 | 4-t-BuPh | 4-t-BuPh | H | H | H |
| 174040 | H | H | 4-t-BuPh | H | H |
| 174041 | H | H | H | 4-t-BuPh | 4-t-BuPh |
| 174042 | 4-t-BuPh | 4-t-BuPh | 4-t-BuPh | H | H |
| 174043 | H | H | 4-t-BuPh | 4-t-BuPh | 4-t-BuPh |
| 174044 | 4-t-BuPh | 4-t-BuPh | H | 4-t-BuPh | 4-t-BuPh |
| 174045 | 1,3-di-t-BuPh | 1,3-di-t-BuPh | H | H | H |
| 174046 | H | H | 1,3-di-t-BuPh | H | H |
| 174047 | H | H | H | 1,3-di-t-BuPh | 1,3-di-t-BuPh |
| 174048 | 1,3-di-t-BuPh | 1,3-di-t-BuPh | 1,3-di-t-BuPh | H | H |
| 174049 | H | H | 1,3-di-t-BuPh | 1,3-di-t-BuPh | 1,3-di-t-BuPh |
| 174050 | 1,3-di-t-BuPh | 1,3-di-t-BuPh | H | 1,3-di-t-BuPh | 1,3-di-t-BuPh |
| 174051 | t-Bu | t-Bu | Ph | H | H |
| 174052 | t-Bu | t-Bu | CN | H | H |
| 174053 | t-Bu | t-Bu | DPA | H | H |
| 174054 | t-Bu | t-Bu | 2-DBF | H | H |
| 174055 | t-Bu | t-Bu | 2-DBT | H | H |
| 174056 | t-Bu | t-Bu | 4-t-BuPh | H | H |
| 174057 | t-Bu | t-Bu | 1,3-di-t-BuPh | H | H |
| 174058 | t-Bu | t-Bu | 9-Cbz | H | H |
| 174059 | t-Bu | t-Bu | Ph | t-Bu | t-Bu |
| 174060 | t-Bu | t-Bu | CN | t-Bu | t-Bu |
| 174061 | t-Bu | t-Bu | DPA | t-Bu | t-Bu |
| 174062 | t-Bu | t-Bu | 2-DBF | t-Bu | t-Bu |
| 174063 | t-Bu | t-Bu | 2-DBT | t-Bu | t-Bu |
| 174064 | t-Bu | t-Bu | 4-t-BuPh | t-Bu | t-Bu |
| 174065 | t-Bu | t-Bu | 1,3-di-t-BuPh | t-Bu | t-Bu |
| 174066 | t-Bu | t-Bu | 9-Cbz | t-Bu | t-Bu |
| 174067 | t-Bu | t-Bu | t-Bu | Ph | Ph |
| 174068 | t-Bu | t-Bu | Ph | Ph | Ph |
| 174069 | t-Bu | t-Bu | CN | Ph | Ph |
| 174070 | t-Bu | t-Bu | DPA | Ph | Ph |
| 174071 | t-Bu | t-Bu | 2-DBF | Ph | Ph |
| 174072 | t-Bu | t-Bu | 2-DBT | Ph | Ph |
| 174073 | t-Bu | t-Bu | 4-t-BuPh | Ph | Ph |
| 174074 | t-Bu | t-Bu | 1,3-di-t-BuPh | Ph | Ph |
| 174075 | t-Bu | t-Bu | 9-Cbz | Ph | Ph |
| 174076 | t-Bu | t-Bu | t-Bu | CN | CN |
| 174077 | t-Bu | t-Bu | Ph | CN | CN |
| 174078 | t-Bu | t-Bu | CN | CN | CN |
| 174079 | t-Bu | t-Bu | DPA | CN | CN |
| 174080 | t-Bu | t-Bu | 2-DBF | CN | CN |
| 174081 | t-Bu | t-Bu | 2-DBT | CN | CN |
| 174082 | t-Bu | t-Bu | 4-t-BuPh | CN | CN |
| 174083 | t-Bu | t-Bu | 1,3-di-t-BuPh | CN | CN |
| 174084 | t-Bu | t-Bu | 9-Cbz | CN | CN |
| 174085 | t-Bu | t-Bu | t-Bu | 9-Cbz | 9-Cbz |
| 174086 | t-Bu | t-Bu | Ph | 9-Cbz | 9-Cbz |
| 174087 | t-Bu | t-Bu | CN | 9-Cbz | 9-Cbz |
| 174088 | t-Bu | t-Bu | DPA | 9-Cbz | 9-Cbz |
| 174089 | t-Bu | t-Bu | 2-DBF | 9-Cbz | 9-Cbz |
| 174090 | t-Bu | t-Bu | 2-DBT | 9-Cbz | 9-Cbz |
| 174091 | t-Bu | t-Bu | 4-t-BuPh | 9-Cbz | 9-Cbz |
| 174092 | t-Bu | t-Bu | 1,3-di-t-BuPh | 9-Cbz | 9-Cbz |
| 174093 | t-Bu | t-Bu | 9-Cbz | 9-Cbz | 9-Cbz |
| 174094 | t-Bu | t-Bu | t-Bu | 4-t-BuPh | 4-t-BuPh |
| 174095 | t-Bu | t-Bu | Ph | 4-t-BuPh | 4-t-BuPh |
| 174096 | t-Bu | t-Bu | CN | 4-t-BuPh | 4-t-BuPh |
| 174097 | t-Bu | t-Bu | DPA | 4-t-BuPh | 4-t-BuPh |
| 174098 | t-Bu | t-Bu | 2-DBF | 4-t-BuPh | 4-t-BuPh |
| 174099 | t-Bu | t-Bu | 2-DBT | 4-t-BuPh | 4-t-BuPh |
| 174100 | t-Bu | t-Bu | 4-t-BuPh | 4-t-BuPh | 4-t-BuPh |
| 174101 | t-Bu | t-Bu | 1,3-di-t-BuPh | 4-t-BuPh | 4-t-BuPh |
| 174102 | t-Bu | t-Bu | 9-Cbz | 4-t-BuPh | 4-t-BuPh |
| 174103 | t-Bu | t-Bu | t-Bu | 1,3-di-t-BuPh | 1,3-di-t-BuPh |
| 174104 | t-Bu | t-Bu | Ph | 1,3-di-t-BuPh | 1,3-di-t-BuPh |
| 174105 | t-Bu | t-Bu | CN | 1,3-di-t-BuPh | 1,3-di-t-BuPh |
| 174106 | t-Bu | t-Bu | DPA | 1,3-di-t-BuPh | 1,3-di-t-BuPh |
| 174107 | t-Bu | t-Bu | 2-DBF | 1,3-di-t-BuPh | 1,3-di-t-BuPh |
| 174108 | t-Bu | t-Bu | 2-DBT | 1,3-di-t-BuPh | 1,3-di-t-BuPh |
| 174109 | t-Bu | t-Bu | 4-t-BuPh | 1,3-di-t-BuPh | 1,3-di-t-BuPh |
| 174110 | t-Bu | t-Bu | 1,3-di-t-BuPh | 1,3-di-t-BuPh | 1,3-di-t-BuPh |
| 174111 | t-Bu | t-Bu | 9-Cbz | 1,3-di-t-BuPh | 1,3-di-t-BuPh |
| 174112 | Ph | Ph | t-Bu | H | H |
| 174113 | Ph | Ph | CN | H | H |
| 174114 | Ph | Ph | DPA | H | H |
| 174115 | Ph | Ph | 2-DBF | H | H |
| 174116 | Ph | Ph | 2-DBT | H | H |
| 174117 | Ph | Ph | 4-t-BuPh | H | H |
| 174118 | Ph | Ph | 1,3-di-t-BuPh | H | H |
| 174119 | Ph | Ph | 9-Cbz | H | H |
| 174120 | Ph | Ph | t-Bu | t-Bu | t-Bu |
| 174121 | Ph | Ph | Ph | t-Bu | t-Bu |
| 174122 | Ph | Ph | CN | t-Bu | t-Bu |
| 174123 | Ph | Ph | DPA | t-Bu | t-Bu |
| 174124 | Ph | Ph | 2-DBF | t-Bu | t-Bu |
| 174125 | Ph | Ph | 2-DBT | t-Bu | t-Bu |
| 174126 | Ph | Ph | 4-t-BuPh | t-Bu | t-Bu |
| 174127 | Ph | Ph | 1,3-di-t-BuPh | t-Bu | t-Bu |
| 174128 | Ph | Ph | 9-Cbz | t-Bu | t-Bu |
| 174129 | Ph | Ph | Ph | Ph | Ph |
| 174130 | Ph | Ph | CN | Ph | Ph |
| 174131 | Ph | Ph | DPA | Ph | Ph |
| 174132 | Ph | Ph | 2-DBF | Ph | Ph |
| 174133 | Ph | Ph | 2-DBT | Ph | Ph |
| 174134 | Ph | Ph | 4-t-BuPh | Ph | Ph |
| 174135 | Ph | Ph | 1,3-di-t-BuPh | Ph | Ph |
| 174136 | Ph | Ph | 9-Cbz | Ph | Ph |
| 174137 | Ph | Ph | t-Bu | CN | CN |
| 174138 | Ph | Ph | Ph | CN | CN |
| 174139 | Ph | Ph | CN | CN | CN |
| 174140 | Ph | Ph | DPA | CN | CN |
| 174141 | Ph | Ph | 2-DBF | CN | CN |
| 174142 | Ph | Ph | 2-DBT | CN | CN |
| 174143 | Ph | Ph | 4-t-BuPh | CN | CN |
| 174144 | Ph | Ph | 1,3-di-t-BuPh | CN | CN |
| 174145 | Ph | Ph | 9-Cbz | CN | CN |
| 174146 | Ph | Ph | t-Bu | 9-Cbz | 9-Cbz |
| 174147 | Ph | Ph | Ph | 9-Cbz | 9-Cbz |
| 174148 | Ph | Ph | CN | 9-Cbz | 9-Cbz |
| 174149 | Ph | Ph | DPA | 9-Cbz | 9-Cbz |
| 174150 | Ph | Ph | 2-DBF | 9-Cbz | 9-Cbz |
| 174151 | Ph | Ph | 2-DBT | 9-Cbz | 9-Cbz |
| 174152 | Ph | Ph | 4-t-BuPh | 9-Cbz | 9-Cbz |
| 174153 | Ph | Ph | 1,3-di-t-BuPh | 9-Cbz | 9-Cbz |
| 174154 | Ph | Ph | 9-Cbz | 9-Cbz | 9-Cbz |
| 174155 | Ph | Ph | t-Bu | 4-t-BuPh | 4-t-BuPh |
| 174156 | Ph | Ph | Ph | 4-t-BuPh | 4-t-BuPh |
| 174157 | Ph | Ph | CN | 4-t-BuPh | 4-t-BuPh |
| 174158 | Ph | Ph | DPA | 4-t-BuPh | 4-t-BuPh |
| 174159 | Ph | Ph | 2-DBF | 4-t-BuPh | 4-t-BuPh |
| 174160 | Ph | Ph | 2-DBT | 4-t-BuPh | 4-t-BuPh |
| 174161 | Ph | Ph | 4-t-BuPh | 4-t-BuPh | 4-t-BuPh |
| 174162 | Ph | Ph | 1,3-di-t-BuPh | 4-t-BuPh | 4-t-BuPh |
| 174163 | Ph | Ph | 9-Cbz | 4-t-BuPh | 4-t-BuPh |
| 174164 | Ph | Ph | t-Bu | 1,3-di-t-BuPh | 1,3-di-t-BuPh |
| 174165 | Ph | Ph | Ph | 1,3-di-t-BuPh | 1,3-di-t-BuPh |
| 174166 | Ph | Ph | CN | 1,3-di-t-BuPh | 1,3-di-t-BuPh |
| 174167 | Ph | Ph | DPA | 1,3-di-t-BuPh | 1,3-di-t-BuPh |
| 174168 | Ph | Ph | 2-DBF | 1,3-di-t-BuPh | 1,3-di-t-BuPh |
| 174169 | Ph | Ph | 2-DBT | 1,3-di-t-BuPh | 1,3-di-t-BuPh |
| 174170 | Ph | Ph | 4-t-BuPh | 1,3-di-t-BuPh | 1,3-di-t-BuPh |
| 174171 | Ph | Ph | 1,3-di-t-BuPh | 1,3-di-t-BuPh | 1,3-di-t-BuPh |
| 174172 | Ph | Ph | 9-Cbz | 1,3-di-t-BuPh | 1,3-di-t-BuPh |
| 174173 | 9-Cbz | 9-Cbz | t-Bu | H | H |
| 174174 | 9-Cbz | 9-Cbz | Ph | H | H |
| 174175 | 9-Cbz | 9-Cbz | CN | H | H |
| 174176 | 9-Cbz | 9-Cbz | DPA | H | H |
| 174177 | 9-Cbz | 9-Cbz | 2-DBF | H | H |
| 174178 | 9-Cbz | 9-Cbz | 2-DBT | H | H |
| 174179 | 9-Cbz | 9-Cbz | 4-t-BuPh | H | H |
| 174180 | 9-Cbz | 9-Cbz | 1,3-di-t-BuPh | H | H |
| 174181 | 9-Cbz | 9-Cbz | t-Bu | t-Bu | t-Bu |
| 174182 | 9-Cbz | 9-Cbz | Ph | t-Bu | t-Bu |
| 174183 | 9-Cbz | 9-Cbz | CN | t-Bu | t-Bu |
| 174184 | 9-Cbz | 9-Cbz | DPA | t-Bu | t-Bu |
| 174185 | 9-Cbz | 9-Cbz | 2-DBF | t-Bu | t-Bu |
| 174186 | 9-Cbz | 9-Cbz | 2-DBT | t-Bu | t-Bu |
| 174187 | 9-Cbz | 9-Cbz | 4-t-BuPh | t-Bu | t-Bu |
| 174188 | 9-Cbz | 9-Cbz | 1,3-di-t-BuPh | t-Bu | t-Bu |
| 174189 | 9-Cbz | 9-Cbz | 9-Cbz | t-Bu | t-Bu |
| 174190 | 9-Cbz | 9-Cbz | t-Bu | Ph | Ph |
| 174191 | 9-Cbz | 9-Cbz | Ph | Ph | Ph |
| 174192 | 9-Cbz | 9-Cbz | CN | Ph | Ph |
| 174193 | 9-Cbz | 9-Cbz | DPA | Ph | Ph |
| 174194 | 9-Cbz | 9-Cbz | 2-DBF | Ph | Ph |
| 174195 | 9-Cbz | 9-Cbz | 2-DBT | Ph | Ph |
| 174196 | 9-Cbz | 9-Cbz | 4-t-BuPh | Ph | Ph |
| 174197 | 9-Cbz | 9-Cbz | 1,3-di-t-BuPh | Ph | Ph |
| 174198 | 9-Cbz | 9-Cbz | 9-Cbz | Ph | Ph |
| 174199 | 9-Cbz | 9-Cbz | t-Bu | CN | CN |
| 174200 | 9-Cbz | 9-Cbz | Ph | CN | CN |
| 174201 | 9-Cbz | 9-Cbz | CN | CN | CN |
| 174202 | 9-Cbz | 9-Cbz | DPA | CN | CN |
| 174203 | 9-Cbz | 9-Cbz | 2-DBF | CN | CN |
| 174204 | 9-Cbz | 9-Cbz | 2-DBT | CN | CN |
| 174205 | 9-Cbz | 9-Cbz | 4-t-BuPh | CN | CN |
| 174206 | 9-Cbz | 9-Cbz | 1,3-di-t-BuPh | CN | CN |
| 174207 | 9-Cbz | 9-Cbz | 9-Cbz | CN | CN |
| 174208 | 9-Cbz | 9-Cbz | t-Bu | 9-Cbz | 9-Cbz |
| 174209 | 9-Cbz | 9-Cbz | Ph | 9-Cbz | 9-Cbz |
| 174210 | 9-Cbz | 9-Cbz | CN | 9-Cbz | 9-Cbz |
| 174211 | 9-Cbz | 9-Cbz | DPA | 9-Cbz | 9-Cbz |
| 174212 | 9-Cbz | 9-Cbz | 2-DBF | 9-Cbz | 9-Cbz |
| 174213 | 9-Cbz | 9-Cbz | 2-DBT | 9-Cbz | 9-Cbz |
| 174214 | 9-Cbz | 9-Cbz | 4-t-BuPh | 9-Cbz | 9-Cbz |
| 174215 | 9-Cbz | 9-Cbz | 1,3-di-t-BuPh | 9-Cbz | 9-Cbz |
| 174216 | 9-Cbz | 9-Cbz | 9-Cbz | 9-Cbz | 9-Cbz |
| 174217 | 9-Cbz | 9-Cbz | t-Bu | 4-t-BuPh | 4-t-BuPh |
| 174218 | 9-Cbz | 9-Cbz | Ph | 4-t-BuPh | 4-t-BuPh |
| 174219 | 9-Cbz | 9-Cbz | CN | 4-t-BuPh | 4-t-BuPh |
| 174220 | 9-Cbz | 9-Cbz | DPA | 4-t-BuPh | 4-t-BuPh |
| 174221 | 9-Cbz | 9-Cbz | 2-DBF | 4-t-BuPh | 4-t-BuPh |
| 174222 | 9-Cbz | 9-Cbz | 2-DBT | 4-t-BuPh | 4-t-BuPh |
| 174223 | 9-Cbz | 9-Cbz | 4-t-BuPh | 4-t-BuPh | 4-t-BuPh |
| 174224 | 9-Cbz | 9-Cbz | 1,3-di-t-BuPh | 4-t-BuPh | 4-t-BuPh |
| 174225 | 9-Cbz | 9-Cbz | 9-Cbz | 4-t-BuPh | 4-t-BuPh |
| 174226 | 9-Cbz | 9-Cbz | t-Bu | 1,3-di-t-BuPh | 1,3-di-t-BuPh |
| 174227 | 9-Cbz | 9-Cbz | Ph | 1,3-di-t-BuPh | 1,3-di-t-BuPh |
| 174228 | 9-Cbz | 9-Cbz | CN | 1,3-di-t-BuPh | 1,3-di-t-BuPh |
| 174229 | 9-Cbz | 9-Cbz | DPA | 1,3-di-t-BuPh | 1,3-di-t-BuPh |
| 174230 | 9-Cbz | 9-Cbz | 2-DBF | 1,3-di-t-BuPh | 1,3-di-t-BuPh |
| 174231 | 9-Cbz | 9-Cbz | 2-DBT | 1,3-di-t-BuPh | 1,3-di-t-BuPh |
| 174232 | 9-Cbz | 9-Cbz | 4-t-BuPh | 1,3-di-t-BuPh | 1,3-di-t-BuPh |
| 174233 | 9-Cbz | 9-Cbz | 1,3-di-t-BuPh | 1,3-di-t-BuPh | 1,3-di-t-BuPh |
| 174234 | 9-Cbz | 9-Cbz | 9-Cbz | 1,3-di-t-BuPh | 1,3-di-t-BuPh |
| 174235 | CN | CN | t-Bu | H | H |
| 174236 | CN | CN | Ph | H | H |
| 174237 | CN | CN | DPA | H | H |
| 174238 | CN | CN | 2-DBF | H | H |
| 174239 | CN | CN | 2-DBT | H | H |
| 174240 | CN | CN | 4-t-BuPh | H | H |
| 174241 | CN | CN | 1,3-di-t-BuPh | H | H |
| 174242 | CN | CN | 9-Cbz | H | H |
| 174243 | CN | CN | t-Bu | t-Bu | t-Bu |
| 174244 | CN | CN | Ph | t-Bu | t-Bu |
| 174245 | CN | CN | CN | t-Bu | t-Bu |
| 174246 | CN | CN | DPA | t-Bu | t-Bu |
| 174247 | CN | CN | 2-DBF | t-Bu | t-Bu |
| 174248 | CN | CN | 2-DBT | t-Bu | t-Bu |
| 174249 | CN | CN | 4-t-BuPh | t-Bu | t-Bu |
| 174250 | CN | CN | 1,3-di-t-BuPh | t-Bu | t-Bu |
| 174251 | CN | CN | 9-Cbz | t-Bu | t-Bu |
| 174252 | CN | CN | t-Bu | Ph | Ph |
| 174253 | CN | CN | Ph | Ph | Ph |
| 174254 | CN | CN | CN | Ph | Ph |
| 174255 | CN | CN | DPA | Ph | Ph |
| 174256 | CN | CN | 2-DBF | Ph | Ph |
| 174257 | CN | CN | 2-DBT | Ph | Ph |
| 174258 | CN | CN | 4-t-BuPh | Ph | Ph |
| 174259 | CN | CN | 1,3-di-t-BuPh | Ph | Ph |
| 174260 | CN | CN | 9-Cbz | Ph | Ph |
| 174261 | CN | CN | t-Bu | CN | CN |
| 174262 | CN | CN | Ph | CN | CN |
| 174263 | CN | CN | CN | CN | CN |
| 174264 | CN | CN | DPA | CN | CN |
| 174265 | CN | CN | 2-DBF | CN | CN |
| 174266 | CN | CN | 2-DBT | CN | CN |
| 174267 | CN | CN | 4-t-BuPh | CN | CN |
| 174268 | CN | CN | 1,3-di-t-BuPh | CN | CN |
| 174269 | CN | CN | 9-Cbz | CN | CN |
| 174270 | CN | CN | t-Bu | 9-Cbz | 9-Cbz |
| 174271 | CN | CN | Ph | 9-Cbz | 9-Cbz |
| 174272 | CN | CN | CN | 9-Cbz | 9-Cbz |
| 174273 | CN | CN | DPA | 9-Cbz | 9-Cbz |
| 174274 | CN | CN | 2-DBF | 9-Cbz | 9-Cbz |
| 174275 | CN | CN | 2-DBT | 9-Cbz | 9-Cbz |
| 174276 | CN | CN | 4-t-BuPh | 9-Cbz | 9-Cbz |
| 174277 | CN | CN | 1,3-di-t-BuPh | 9-Cbz | 9-Cbz |
| 174278 | CN | CN | 9-Cbz | 9-Cbz | 9-Cbz |
| 174279 | CN | CN | t-Bu | 4-t-BuPh | 4-t-BuPh |
| 174280 | CN | CN | Ph | 4-t-BuPh | 4-t-BuPh |
| 174281 | CN | CN | CN | 4-t-BuPh | 4-t-BuPh |
| 174282 | CN | CN | DPA | 4-t-BuPh | 4-t-BuPh |
| 174283 | CN | CN | 2-DBF | 4-t-BuPh | 4-t-BuPh |
| 174284 | CN | CN | 2-DBT | 4-t-BuPh | 4-t-BuPh |
| 174285 | CN | CN | 4-t-BuPh | 4-t-BuPh | 4-t-BuPh |
| 174286 | CN | CN | 1,3-di-t-BuPh | 4-t-BuPh | 4-t-BuPh |
| 174287 | CN | CN | 9-Cbz | 4-t-BuPh | 4-t-BuPh |
| 174288 | CN | CN | t-Bu | 1,3-di-t-BuPh | 1,3-di-t-BuPh |
| 174289 | CN | CN | Ph | 1,3-di-t-BuPh | 1,3-di-t-BuPh |
| 174290 | CN | CN | CN | 1,3-di-t-BuPh | 1,3-di-t-BuPh |
| 174291 | CN | CN | DPA | 1,3-di-t-BuPh | 1,3-di-t-BuPh |
| 174292 | CN | CN | 2-DBF | 1,3-di-t-BuPh | 1,3-di-t-BuPh |
| 174293 | CN | CN | 2-DBT | 1,3-di-t-BuPh | 1,3-di-t-BuPh |
| 174294 | CN | CN | 4-t-BuPh | 1,3-di-t-BuPh | 1,3-di-t-BuPh |
| 174295 | CN | CN | 1,3-di-t-BuPh | 1,3-di-t-BuPh | 1,3-di-t-BuPh |
| 174296 | CN | CN | 9-Cbz | 1,3-di-t-BuPh | 1,3-di-t-BuPh |
| 174297 | 4-t-BuPh | 4-t-BuPh | t-Bu | H | H |
| 174298 | 4-t-BuPh | 4-t-BuPh | Ph | H | H |
| 174299 | 4-t-BuPh | 4-t-BuPh | CN | H | H |
| 174300 | 4-t-BuPh | 4-t-BuPh | DPA | H | H |
| 174301 | 4-t-BuPh | 4-t-BuPh | 2-DBF | H | H |
| 174302 | 4-t-BuPh | 4-t-BuPh | 2-DBT | H | H |
| 174303 | 4-t-BuPh | 4-t-BuPh | 1,3-di-t-BuPh | H | H |
| 174304 | 4-t-BuPh | 4-t-BuPh | 9-Cbz | H | H |
| 174305 | 4-t-BuPh | 4-t-BuPh | t-Bu | t-Bu | t-Bu |
| 174306 | 4-t-BuPh | 4-t-BuPh | Ph | t-Bu | t-Bu |
| 174307 | 4-t-BuPh | 4-t-BuPh | CN | t-Bu | t-Bu |
| 174308 | 4-t-BuPh | 4-t-BuPh | DPA | t-Bu | t-Bu |
| 174309 | 4-t-BuPh | 4-t-BuPh | 2-DBF | t-Bu | t-Bu |
| 174310 | 4-t-BuPh | 4-t-BuPh | 2-DBT | t-Bu | t-Bu |
| 174311 | 4-t-BuPh | 4-t-BuPh | 4-t-BuPh | t-Bu | t-Bu |
| 174312 | 4-t-BuPh | 4-t-BuPh | 1,3-di-t-BuPh | t-Bu | t-Bu |
| 174313 | 4-t-BuPh | 4-t-BuPh | 9-Cbz | t-Bu | t-Bu |
| 174314 | 4-t-BuPh | 4-t-BuPh | t-Bu | Ph | Ph |
| 174315 | 4-t-BuPh | 4-t-BuPh | Ph | Ph | Ph |
| 174316 | 4-t-BuPh | 4-t-BuPh | CN | Ph | Ph |
| 174317 | 4-t-BuPh | 4-t-BuPh | DPA | Ph | Ph |
| 174318 | 4-t-BuPh | 4-t-BuPh | 2-DBF | Ph | Ph |
| 174319 | 4-t-BuPh | 4-t-BuPh | 2-DBT | Ph | Ph |
| 174320 | 4-t-BuPh | 4-t-BuPh | 4-t-BuPh | Ph | Ph |
| 174321 | 4-t-BuPh | 4-t-BuPh | 1,3-di-t-BuPh | Ph | Ph |
| 174322 | 4-t-BuPh | 4-t-BuPh | 9-Cbz | Ph | Ph |
| 174323 | 4-t-BuPh | 4-t-BuPh | t-Bu | CN | CN |
| 174324 | 4-t-BuPh | 4-t-BuPh | Ph | CN | CN |
| 174325 | 4-t-BuPh | 4-t-BuPh | CN | CN | CN |
| 174326 | 4-t-BuPh | 4-t-BuPh | DPA | CN | CN |
| 174327 | 4-t-BuPh | 4-t-BuPh | 2-DBF | CN | CN |
| 174328 | 4-t-BuPh | 4-t-BuPh | 2-DBT | CN | CN |
| 174329 | 4-t-BuPh | 4-t-BuPh | 4-t-BuPh | CN | CN |
| 174330 | 4-t-BuPh | 4-t-BuPh | 1,3-di-t-BuPh | CN | CN |
| 174331 | 4-t-BuPh | 4-t-BuPh | 9-Cbz | CN | CN |
| 174332 | 4-t-BuPh | 4-t-BuPh | t-Bu | 9-Cbz | 9-Cbz |
| 174333 | 4-t-BuPh | 4-t-BuPh | Ph | 9-Cbz | 9-Cbz |
| 174334 | 4-t-BuPh | 4-t-BuPh | CN | 9-Cbz | 9-Cbz |
| 174335 | 4-t-BuPh | 4-t-BuPh | DPA | 9-Cbz | 9-Cbz |
| 174336 | 4-t-BuPh | 4-t-BuPh | 2-DBF | 9-Cbz | 9-Cbz |
| 174337 | 4-t-BuPh | 4-t-BuPh | 2-DBT | 9-Cbz | 9-Cbz |
| 174338 | 4-t-BuPh | 4-t-BuPh | 4-t-BuPh | 9-Cbz | 9-Cbz |
| 174339 | 4-t-BuPh | 4-t-BuPh | 1,3-di-t-BuPh | 9-Cbz | 9-Cbz |
| 174340 | 4-t-BuPh | 4-t-BuPh | 9-Cbz | 9-Cbz | 9-Cbz |
| 174341 | 4-t-BuPh | 4-t-BuPh | t-Bu | 4-t-BuPh | 4-t-BuPh |
| 174342 | 4-t-BuPh | 4-t-BuPh | Ph | 4-t-BuPh | 4-t-BuPh |
| 174343 | 4-t-BuPh | 4-t-BuPh | CN | 4-t-BuPh | 4-t-BuPh |
| 174344 | 4-t-BuPh | 4-t-BuPh | DPA | 4-t-BuPh | 4-t-BuPh |
| 174345 | 4-t-BuPh | 4-t-BuPh | 2-DBF | 4-t-BuPh | 4-t-BuPh |
| 174346 | 4-t-BuPh | 4-t-BuPh | 2-DBT | 4-t-BuPh | 4-t-BuPh |
| 174347 | 4-t-BuPh | 4-t-BuPh | 4-t-BuPh | 4-t-BuPh | 4-t-BuPh |
| 174348 | 4-t-BuPh | 4-t-BuPh | 1,3-di-t-BuPh | 4-t-BuPh | 4-t-BuPh |
| 174349 | 4-t-BuPh | 4-t-BuPh | 9-Cbz | 4-t-BuPh | 4-t-BuPh |
| 174350 | 4-t-BuPh | 4-t-BuPh | t-Bu | 1,3-di-t-BuPh | 1,3-di-t-BuPh |
| 174351 | 4-t-BuPh | 4-t-BuPh | Ph | 1,3-di-t-BuPh | 1,3-di-t-BuPh |
| 174352 | 4-t-BuPh | 4-t-BuPh | CN | 1,3-di-t-BuPh | 1,3-di-t-BuPh |
| 174353 | 4-t-BuPh | 4-t-BuPh | DPA | 1,3-di-t-BuPh | 1,3-di-t-BuPh |
| 174354 | 4-t-BuPh | 4-t-BuPh | 2-DBF | 1,3-di-t-BuPh | 1,3-di-t-BuPh |
| 174355 | 4-t-BuPh | 4-t-BuPh | 2-DBT | 1,3-di-t-BuPh | 1,3-di-t-BuPh |
| 174356 | 4-t-BuPh | 4-t-BuPh | 4-t-BuPh | 1,3-di-t-BuPh | 1,3-di-t-BuPh |
| 174357 | 4-t-BuPh | 4-t-BuPh | 1,3-di-t-BuPh | 1,3-di-t-BuPh | 1,3-di-t-BuPh |
| 174358 | 4-t-BuPh | 4-t-BuPh | 9-Cbz | 1,3-di-t-BuPh | 1,3-di-t-BuPh |
| 174359 | 1,3-di-t-BuPh | 1,3-di-t-BuPh | t-Bu | H | H |
| 174360 | 1,3-di-t-BuPh | 1,3-di-t-BuPh | Ph | H | H |
| 174361 | 1,3-di-t-BuPh | 1,3-di-t-BuPh | CN | H | H |
| 174362 | 1,3-di-t-BuPh | 1,3-di-t-BuPh | DPA | H | H |
| 174363 | 1,3-di-t-BuPh | 1,3-di-t-BuPh | 2-DBF | H | H |
| 174364 | 1,3-di-t-BuPh | 1,3-di-t-BuPh | 2-DBT | H | H |
| 174365 | 1,3-di-t-BuPh | 1,3-di-t-BuPh | 4-t-BuPh | H | H |
| 174366 | 1,3-di-t-BuPh | 1,3-di-t-BuPh | 9-Cbz | H | H |
| 174367 | 1,3-di-t-BuPh | 1,3-di-t-BuPh | t-Bu | t-Bu | t-Bu |
| 174368 | 1,3-di-t-BuPh | 1,3-di-t-BuPh | Ph | t-Bu | t-Bu |
| 174369 | 1,3-di-t-BuPh | 1,3-di-t-BuPh | CN | t-Bu | t-Bu |
| 174370 | 1,3-di-t-BuPh | 1,3-di-t-BuPh | DPA | t-Bu | t-Bu |
| 174371 | 1,3-di-t-BuPh | 1,3-di-t-BuPh | 2-DBF | t-Bu | t-Bu |
| 174372 | 1,3-di-t-BuPh | 1,3-di-t-BuPh | 2-DBT | t-Bu | t-Bu |
| 174373 | 1,3-di-t-BuPh | 1,3-di-t-BuPh | 4-t-BuPh | t-Bu | t-Bu |
| 174374 | 1,3-di-t-BuPh | 1,3-di-t-BuPh | 1,3-di-t-BuPh | t-Bu | t-Bu |
| 174375 | 1,3-di-t-BuPh | 1,3-di-t-BuPh | 9-Cbz | t-Bu | t-Bu |
| 174376 | 1,3-di-t-BuPh | 1,3-di-t-BuPh | t-Bu | Ph | Ph |
| 174377 | 1,3-di-t-BuPh | 1,3-di-t-BuPh | Ph | Ph | Ph |
| 174378 | 1,3-di-t-BuPh | 1,3-di-t-BuPh | CN | Ph | Ph |
| 174379 | 1,3-di-t-BuPh | 1,3-di-t-BuPh | DPA | Ph | Ph |
| 174380 | 1,3-di-t-BuPh | 1,3-di-t-BuPh | 2-DBF | Ph | Ph |
| 174381 | 1,3-di-t-BuPh | 1,3-di-t-BuPh | 2-DBT | Ph | Ph |
| 174382 | 1,3-di-t-BuPh | 1,3-di-t-BuPh | 4-t-BuPh | Ph | Ph |
| 174383 | 1,3-di-t-BuPh | 1,3-di-t-BuPh | 1,3-di-t-BuPh | Ph | Ph |
| 174384 | 1,3-di-t-BuPh | 1,3-di-t-BuPh | 9-Cbz | Ph | Ph |
| 174385 | 1,3-di-t-BuPh | 1,3-di-t-BuPh | t-Bu | CN | CN |
| 174386 | 1,3-di-t-BuPh | 1,3-di-t-BuPh | Ph | CN | CN |
| 174387 | 1,3-di-t-BuPh | 1,3-di-t-BuPh | CN | CN | CN |
| 174388 | 1,3-di-t-BuPh | 1,3-di-t-BuPh | DPA | CN | CN |
| 174389 | 1,3-di-t-BuPh | 1,3-di-t-BuPh | 2-DBF | CN | CN |
| 174390 | 1,3-di-t-BuPh | 1,3-di-t-BuPh | 2-DBT | CN | CN |
| 174391 | 1,3-di-t-BuPh | 1,3-di-t-BuPh | 4-t-BuPh | CN | CN |
| 174392 | 1,3-di-t-BuPh | 1,3-di-t-BuPh | 1,3-di-t-BuPh | CN | CN |
| 174393 | 1,3-di-t-BuPh | 1,3-di-t-BuPh | 9-Cbz | CN | CN |
| 174394 | 1,3-di-t-BuPh | 1,3-di-t-BuPh | t-Bu | 9-Cbz | 9-Cbz |
| 174395 | 1,3-di-t-BuPh | 1,3-di-t-BuPh | Ph | 9-Cbz | 9-Cbz |
| 174396 | 1,3-di-t-BuPh | 1,3-di-t-BuPh | CN | 9-Cbz | 9-Cbz |
| 174397 | 1,3-di-t-BuPh | 1,3-di-t-BuPh | DPA | 9-Cbz | 9-Cbz |
| 174398 | 1,3-di-t-BuPh | 1,3-di-t-BuPh | 2-DBF | 9-Cbz | 9-Cbz |
| 174399 | 1,3-di-t-BuPh | 1,3-di-t-BuPh | 2-DBT | 9-Cbz | 9-Cbz |
| 174400 | 1,3-di-t-BuPh | 1,3-di-t-BuPh | 4-t-BuPh | 9-Cbz | 9-Cbz |
| 174401 | 1,3-di-t-BuPh | 1,3-di-t-BuPh | 1,3-di-t-BuPh | 9-Cbz | 9-Cbz |
| 174402 | 1,3-di-t-BuPh | 1,3-di-t-BuPh | 9-Cbz | 9-Cbz | 9-Cbz |
| 174403 | 1,3-di-t-BuPh | 1,3-di-t-BuPh | t-Bu | 4-t-BuPh | 4-t-BuPh |
| 174404 | 1,3-di-t-BuPh | 1,3-di-t-BuPh | Ph | 4-t-BuPh | 4-t-BuPh |
| 174405 | 1,3-di-t-BuPh | 1,3-di-t-BuPh | CN | 4-t-BuPh | 4-t-BuPh |
| 174406 | 1,3-di-t-BuPh | 1,3-di-t-BuPh | DPA | 4-t-BuPh | 4-t-BuPh |
| 174407 | 1,3-di-t-BuPh | 1,3-di-t-BuPh | 2-DBF | 4-t-BuPh | 4-t-BuPh |
| 174408 | 1,3-di-t-BuPh | 1,3-di-t-BuPh | 2-DBT | 4-t-BuPh | 4-t-BuPh |
| 174409 | 1,3-di-t-BuPh | 1,3-di-t-BuPh | 4-t-BuPh | 4-t-BuPh | 4-t-BuPh |
| 174410 | 1,3-di-t-BuPh | 1,3-di-t-BuPh | 1,3-di-t-BuPh | 4-t-BuPh | 4-t-BuPh |
| 174411 | 1,3-di-t-BuPh | 1,3-di-t-BuPh | 9-Cbz | 4-t-BuPh | 4-t-BuPh |
| 174412 | 1,3-di-t-BuPh | 1,3-di-t-BuPh | t-Bu | 1,3-di-t-BuPh | 1,3-di-t-BuPh |
| 174413 | 1,3-di-t-BuPh | 1,3-di-t-BuPh | Ph | 1,3-di-t-BuPh | 1,3-di-t-BuPh |
| 174414 | 1,3-di-t-BuPh | 1,3-di-t-BuPh | CN | 1,3-di-t-BuPh | 1,3-di-t-BuPh |
| 174415 | 1,3-di-t-BuPh | 1,3-di-t-BuPh | DPA | 1,3-di-t-BuPh | 1,3-di-t-BuPh |
| 174416 | 1,3-di-t-BuPh | 1,3-di-t-BuPh | 2-DBF | 1,3-di-t-BuPh | 1,3-di-t-BuPh |
| 174417 | 1,3-di-t-BuPh | 1,3-di-t-BuPh | 2-DBT | 1,3-di-t-BuPh | 1,3-di-t-BuPh |
| 174418 | 1,3-di-t-BuPh | 1,3-di-t-BuPh | 4-t-BuPh | 1,3-di-t-BuPh | 1,3-di-t-BuPh |
| 174419 | 1,3-di-t-BuPh | 1,3-di-t-BuPh | 1,3-di-t-BuPh | 1,3-di-t-BuPh | 1,3-di-t-BuPh |
| 174420 | 1,3-di-t-BuPh | 1,3-di-t-BuPh | 9-Cbz | 1,3-di-t-BuPh | 1,3-di-t-BuPh |

In Tables 1 to 10, “H” represents hydrogen, “CN” represents a cyano group, “Me” represents a methyl group, “t-Bu” represents a t-butyl group, “Ph” represents a phenyl group, and “4-t-BuPh”, “1,3-di-t-BuPh”, “9-Cbz”, “DPA”, “2-DBF”, and “2-DBT” may be represented by as follows:

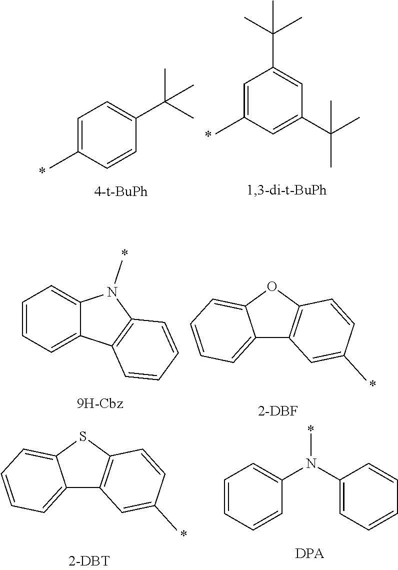

wherein, in “4-t-BuPh”, “1,3-di-t-BuPh”, “9-Cbz”, “DPA”, “2-DBF”, and “2-DBT”, * indicates a binding site to an adjacent atom.

In some embodiments, the heterocyclic compound may be represented by one of Formulae 11, 12, and 14 to 17, in Formula 11, ii)*1 may be X₁₃, *2 may be X₁₄, *3 may be Y₁₄, and *4 may be Y₁₅, or v)*1 may be X₁₄, *2 may be X₁₅, *3 may be Y₁₄, and *4 may be Y₁₅; in Formula 12, i)*1 may be X₁₃, *2 may be X₁₄, *3 may be Y₁₃, and *4 may be Y₁₄, ii)*1 may be X₁₃, *2 may be X₁₄, *3 may be Y₁₄, and *4 may be Y₁₅, or iv)*1 may be X₁₄, *2 may be X₁₅, *3 may be Y₁₄, and *4 may be Y₁₅; in Formula 14, i)*5 may be X₁₃, *6 may be X₁₄, *7 may be Z₁₄, and *8 may be Z₁₅, iii)*5 may be X₁₄, *6 may be X₁₅, *7 may be Z₁₄, and *8 may be Z₁₅, or iv)*5 may be X₁₄, *6 may be X₁₅, *7 may be Z₁₅, and *8 may be Z₁₆; in Formula 15, iii)*5 may be X₁₄, *6 may be X₁₅, *7 may be Z₁₄, and *8 may be Z₁₅; in Formula 16, iii)*5 may be X₁₄, *6 may be X₁₅, *7 may be Z₁₄, and *8 may be Z₁₅; or iv)*5 may be X₁₄, *6 may be X₁₅, *7 may be Z₁₅, and *8 may be Z₁₆, and in Formula 17, iv)*5 may be X₁₄, *6 may be X₁₅, *7 may be Z₁₅, and *8 may be Z₁₆,

In some embodiments, the heterocyclic compound may be represented by one of Formulae 11-2, 11-5, 12-1, 12-2, 12-4, 14-1, 14-3, 14-4, 15-3, 16-3, 16-4, and 17-4.

In some embodiments, in Formulae 11-2, 11-5, 12-1, 12-2, 12-4, 14-1, 14-3, 14-4, 15-3, 16-3, 16-4, and 17-4, R₁₁ₐ to R_{11c}, R₁₂ₐ to R₁₂d, R₁₃ₐ and R_{13b}, R₁₄ₐ to R_{14c}, R₁₅ₐ to R₁₅d, R₁₆ₐ and R_{16b}, R₁₇ₐ, R₁₈ₐ to R₁₈d, and R₁₉ₐ to R_{19c} may each independently be hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, —SF₅, a C₁-C₂₀ alkyl group, a C₁-C₂₀ alkenyl group, a C₁-C₂₀ alkoxy group, or a C₁-C₂₀ alkylthio group;

a C₁-C₂₀ alkyl group, a C₁-C₂₀ alkenyl group, a C₁-C₂₀ alkoxy group, or a C₁-C₂₀ alkylthio group, each substituted with deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₁₀ alkyl group, a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a bicyclo[1.1.1]pentyl group, a bicyclo[2.1.1]hexyl group, a bicyclo[2.2.1]heptyl group, a bicyclo[2.2.2]octyl group, a (C₁-C₂₀ alkyl)cyclopentyl group, a (C₁-C₂₀ alkyl)cyclohexyl group, a (C₁-C₂₀ alkyl)cycloheptyl group, a (C₁-C₂₀ alkyl)cyclooctyl group, a (C₁-C₂₀ alkyl)adamantanyl group, a (C₁-C₂₀ alkyl)norbornanyl group, a (C₁-C₂₀ alkyl)norbornenyl group, a (C₁-C₂₀ alkyl)cyclopentenyl group, a (C₁-C₂₀ alkyl)cyclohexenyl group, a (C₁-C₂₀ alkyl)cycloheptenyl group, a (C₁-C₂₀ alkyl)bicyclo[1.1.1]pentyl group, a (C₁-C₂₀ alkyl)bicyclo[2.1.1]hexyl group, a (C₁-C₂₀ alkyl)bicyclo[2.2.1]heptyl group, a (C₁-C₂₀ alkyl)bicyclo[2.2.2]octyl group, a phenyl group, a (C₁-C₂₀ alkyl)phenyl group, a biphenyl group, a terphenyl group, a naphthyl group, a pyridinyl group, a pyrimidinyl group, or any combination thereof;

a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a bicyclo[1.1.1]pentyl group, a bicyclo[2.1.1]hexyl group, a bicyclo[2.2.1]heptyl group, a bicyclo[2.2.2]octyl group, a phenyl group, a (C₁-C₂₀ alkyl)phenyl group, a biphenyl group, a terphenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, an isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, an imidazopyrimidinyl group, an azacarbazolyl group, an azadibenzofuranyl group, or an azadibenzothiophenyl group, each unsubstituted or substituted with deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a deuterated C₂-C₂₀ alkyl group, a C₁-C₂₀ alkoxy group, a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a bicyclo[1.1.1]pentyl group, a bicyclo[2.1.1]hexyl group, a bicyclo[2.2.1]heptyl group, a bicyclo[2.2.2]octyl group, a (C₁-C₂₀ alkyl)cyclopentyl group, a (C₁-C₂₀ alkyl)cyclohexyl group, a (C₁-C₂₀ alkyl)cycloheptyl group, a (C₁-C₂₀ alkyl)cyclooctyl group, a (C₁-C₂₀ alkyl)adamantanyl group, a (C₁-C₂₀ alkyl)norbornanyl group, a (C₁-C₂₀ alkyl)norbornenyl group, a (C₁-C₂₀ alkyl)cyclopentenyl group, a (C₁-C₂₀ alkyl)cyclohexenyl group, a (C₁-C₂₀ alkyl)cycloheptenyl group, a (C₁-C₂₀ alkyl)bicyclo[1.1.1]pentyl group, a (C₁-C₂₀ alkyl)bicyclo[2.1.1]hexyl group, a (C₁-C₂₀ alkyl)bicyclo[2.2.1]heptyl group, a (C₁-C₂₀ alkyl)bicyclo[2.2.2]octyl group, a phenyl group, a (C₁-C₂₀ alkyl)phenyl group, a biphenyl group, a terphenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, an isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, an imidazopyrimidinyl group, an azacarbazolyl group, an azadibenzofuranyl group, an azadibenzothiophenyl group, or any combination thereof; or

—Si(Q₁)(Q₂)(Q₃), —Ge(Q₁)(Q₂)(Q₃), —C(Q₁)(Q₂)(Q₃), —B(Q₁)(Q₂), or —N(Q₁)(Q₂),

wherein Q₁ to Q₃ may each independently be:

deuterium, —F, —CH₃, —CD₃, —CD₂H, —CDH₂, —CH₂CH₃, —CH₂CD₃, —CH₂CD₂H, —CH₂CDH₂, —CHDCH₃, —CHDCD₂H, —CHDCDH₂, —CHDCD₃, —CD₂CD₃, —CD₂CD₂H, —CD₂CDH₂, —CF₃, —CF₂H, —CFH₂, —CH₂CF₃, —CH₂CF₂H, —CH₂CFH₂, —CHFCH₃, —CHFCF₂H, —CHFCFH₂, —CHFCF₃, —CF₂CF₃, —CF₂CF₂H, or —CF₂CFH₂; or

an n-propyl group, an iso-propyl group, an n-butyl group, a sec-butyl group, an isobutyl group, a tert-butyl group, an n-pentyl group, a tert-pentyl group, a neopentyl group, an isopentyl group, a sec-pentyl group, a 3-pentyl group, a sec-isopentyl group, a phenyl group, a biphenyl group, or a naphthyl group, each unsubstituted or substituted with deuterium, —F, a C₁-C₁₀ alkyl group, a phenyl group, or any combination thereof.

In some embodiments, in Formulae 11-2, 11-5, 12-1, 12-2, 12-4, 14-1, 14-3, 14-4, 15-3, 16-3, 16-4, and 17-4, R₁₁ₐ to R_{11c}, R₁₂ₐ to R₁₂d, R₁₃ₐ and R_{13b}, R₁₄ₐ to R_{14c}, R₁₅ₐ to R₁₅d, R₁₆ₐ and R_{16b}, R₁₇ₐ, R₁₈ₐ to R₁₈d, and R₁₉ₐ to R_{19c} may each independently be hydrogen, deuterium, —F, —CH₃, —CD₃, —CD₂H, —CDH₂, —CF₃, —CF₂H, —CFH₂, a C₂-C₁₀ alkenyl group, a C₁-C₁₀ alkoxy group, a C₁-C₁₀ alkylthio group, a group represented by one of Formulae 9-1 to 9-39, a group represented by one of Formulae 9-1 to 9-39 in which at least one hydrogen is substituted with deuterium, a group represented by one of Formulae 9-1 to 9-39 in which at least one hydrogen is substituted with —F, a group represented by one of Formulae 9-201 to 9-236, a group represented by one of Formulae 9-201 to 9-236 in which at least one hydrogen is substituted with deuterium, a group represented by one of Formulae 9-201 to 9-236 in which at least one hydrogen is substituted with —F, a group represented by one of Formulae 10-1 to 10-130, a group represented by one of Formulae 10-1 to 10-130 in which at least one hydrogen is substituted with deuterium, a group represented by one of Formulae 10-1 to 10-130 in which at least one hydrogen is substituted with —F, a group represented by one of Formulae 10-201 to 10-358, a group represented by one of Formulae 10-201 to 10-358 in which at least one hydrogen is substituted with deuterium, a group represented by one of Formulae 10-201 to 10-358 in which at least one hydrogen is substituted with —F, —Si(Q₁)(Q₂)(Q₃), —Ge(Q₁)(Q₂)(Q₃), —C(Q₁)(Q₂)(Q₃), —B(Q₁)(Q₂), or —N(Q₁)(Q₂),

wherein Q₁ to Q₃ may each independently be:

deuterium, —F, —CH₃, —CD₃, —CD₂H, —CDH₂, —CH₂CH₃, —CH₂CD₃, —CH₂CD₂H, —CH₂CDH₂, —CHDCH₃, —CHDCD₂H, —CHDCDH₂, —CHDCD₃, —CD₂CD₃, —CD₂CD₂H, —CD₂CDH₂, —CF₃, —CF₂H, —CFH₂, —CH₂CF₃, —CH₂CF₂H, —CH₂CFH₂, —CHFCH₃, —CHFCF₂H, —CHFCFH₂, —CHFCF₃, —CF₂CF₃, —CF₂CF₂H, or —CF₂CFH₂; or

an n-propyl group, an iso-propyl group, an n-butyl group, a sec-butyl group, an isobutyl group, a tert-butyl group, an n-pentyl group, a tert-pentyl group, a neopentyl group, an isopentyl group, a sec-pentyl group, a 3-pentyl group, a sec-isopentyl group, a phenyl group, a biphenyl group, or a naphthyl group, each unsubstituted or substituted with deuterium, —F, a C₁-C₁₀ alkyl group, a phenyl group, or any combination thereof.

In some embodiments, in Formulae 11-2, 11-5, 12-1, 12-2, 12-4, 14-1, 14-3, 14-4, 15-3, 16-3, 16-4, and 17-4, R₁₁ₐ to R_{11c}, R₁₂ₐ to R₁₂d, R₁₃ₐ and R_{13b}, R₁₄ₐ to R_{14c}, R₁₅ₐ to R₁₅d, R₁₆ₐ and R_{16b}, R₁₇ₐ, R₁₈ₐ to R₁₈d, and R₁₉ₐ to R_{19c} may each independently be hydrogen, deuterium, —F, —CH₃, —CDs, —CD₂H, —CDH₂, —CF₃, —CF₂H, —CFH₂, a group represented by one of Formulae 9-1 to 9-39, a group represented by one of Formulae 9-1 to 9-39 in which at least one hydrogen is substituted with deuterium, a group represented by one of Formulae 9-1 to 9-39 in which at least one hydrogen is substituted with —F, a group represented by one of Formulae 10-1 to 10-130, a group represented by one of Formulae 10-1 to 10-130 in which at least one hydrogen is substituted with deuterium, a group represented by one of Formulae 10-1 to 10-130 in which at least one hydrogen is substituted with —F, —Si(Q₁)(Q₂)(Q₃), —Ge(Q₁)(Q₂)(Q₃), —C(Q₁)(Q₂)(Q₃), —B(Q₁)(Q₂), or —N(Q₁)(Q₂),

wherein Q₁ to Q₃ may each independently be:

deuterium, —F, —CH₃, —CDs, —CD₂H, —CDH₂, —CH₂CH₃, —CH₂CD₃, —CH₂CD₂H, —CH₂CDH₂, —CHDCH₃, —CHDCD₂H, —CHDCDH₂, —CHDCD₃, —CD₂CD₃, —CD₂CD₂H, —CD₂CDH₂, —CF₃, —CF₂H, —CFH₂, —CH₂CF₃, —CH₂CF₂H, —CH₂CFH₂, —CHFCH₃, —CHFCF₂H, —CHFCFH₂, —CHFCF₃, —CF₂CF₃, —CF₂CF₂H, or —CF₂CFH₂; or

an n-propyl group, an iso-propyl group, an n-butyl group, a sec-butyl group, an isobutyl group, a tert-butyl group, an n-pentyl group, a tert-pentyl group, a neopentyl group, an isopentyl group, a sec-pentyl group, a 3-pentyl group, a sec-isopentyl group, a phenyl group, a biphenyl group, or a naphthyl group, each unsubstituted or substituted with deuterium, —F, a C₁-C₁₀ alkyl group, a phenyl group, or any combination thereof.

In some embodiments, in Formulae 11-2, 11-5, 12-1, 12-2, 12-4, 14-1, 14-3, 14-4, 15-3, 16-3, 16-4, and 17-4, at least one of R_{11b}, R_{12c}, R_{14b}, R_{18b}, and R_{19b} in Formulae 11-2 and 11-5, at least one of R_{11b}, R_{12c}, R_{14b}, R_{18c}, and R_{19b} in Formulae 12-1, 12-2, and 12-4, and at least one of R_{11b}, R_{12c}, R_{15c}, R_{18b}, and R_{19b} in Formulae 14-1, 14-3, 14-4, 15-3, 16-3, 16-4, and 17-4 may be —CH₃, —CDs, —CD₂H, —CDH₂, —CF₃, —CF₂H, —CFH₂, a group represented by one of Formulae 9-1 to 9-39, a group represented by one of Formulae 9-1 to 9-39 in which at least one hydrogen is substituted with deuterium, a group represented by one of Formulae 9-1 to 9-39 in which at least one hydrogen is substituted with —F, a group represented by one of Formulae 10-1 to 10-130, a group represented by one of Formulae 10-1 to 10-130 in which at least one hydrogen is substituted with deuterium, a group represented by one of Formulae 10-1 to 10-130 in which at least one hydrogen is substituted with —F, —Si(Q₁)(Q₂)(Q₃), —Ge(Q₁)(Q₂)(Q₃), —C(Q₁)(Q₂)(Q₃), —B(Q₁)(Q₂), or —N(Q₁)(Q₂),

wherein Q₁ to Q₃ may each independently be:

deuterium, —F, —CH₃, —CD₃, —CD₂H, —CDH₂, —CH₂CH₃, —CH₂CD₃, —CH₂CD₂H, —CH₂CDH₂, —CHDCH₃, —CHDCD₂H, —CHDCDH₂, —CHDCD₃, —CD₂CD₃, —CD₂CD₂H, —CD₂CDH₂, —CF₃, —CF₂H, —CFH₂, —CH₂CF₃, —CH₂CF₂H, —CH₂CFH₂, —CHFCH₃, —CHFCF₂H, —CHFCFH₂, —CHFCF₃, —CF₂CF₃, —CF₂CF₂H, or —CF₂CFH₂; or

an n-propyl group, an iso-propyl group, an n-butyl group, a sec-butyl group, an isobutyl group, a tert-butyl group, an n-pentyl group, a tert-pentyl group, a neopentyl group, an isopentyl group, a sec-pentyl group, a 3-pentyl group, a sec-isopentyl group, a phenyl group, a biphenyl group, or a naphthyl group, each unsubstituted or substituted with deuterium, —F, a C₁-C₁₀ alkyl group, a phenyl group, or any combination thereof.

In some embodiments, in Formulae 11-2, 11-5, 12-1, 12-2, 12-4, 14-1, 14-3, 14-4, 15-3, 16-3, 16-4, and 17-4, at least one of R_{11b}, R_{12c}, R_{14b}, R_{18b}, and R_{19b} in Formulae 11-2 and 11-5, at least one of R_{11b}, R_{12c}, R_{14b}, R_{18c}, and R_{19b} in Formulae 12-1, 12-2, and 12-4, and at least one of R_{11b}, R_{12c}, R_{15c}, R_{18b}, and R_{19b} in Formulae 14-1, 14-3, 14-4, 15-3, 16-3, 16-4, and 17-4 may be —CH₃, —CD₃, —CD₂H, —CDH₂, —CF₃, —CF₂H, —CFH₂, a group represented by one of Formulae 9-1 to 9-39, a group represented by one of Formulae 9-1 to 9-39 in which at least one hydrogen is substituted with deuterium, a group represented by one of Formulae 9-1 to 9-39 in which at least one hydrogen is substituted with —F, a group represented by one of Formulae 10-1 to 10-130, a group represented by one of Formulae 10-1 to 10-130 in which at least one hydrogen is substituted with deuterium, a group represented by one of Formulae 10-1 to 10-130 in which at least one hydrogen is substituted with —F, —Si(Q₁)(Q₂)(Q₃), —Ge(Q₁)(Q₂)(Q₃), —C(Q₁)(Q₂)(Q₃), —B(Q₁)(Q₂), or —N(Q₁)(Q₂),

wherein Q₁ to Q₃ may each independently be:

deuterium, —F, —CH₃, —CD₃, —CD₂H, —CDH₂, —CH₂CH₃, —CH₂CD₃, —CH₂CD₂H, —CH₂CDH₂, —CHDCH₃, —CHDCD₂H, —CHDCDH₂, —CHDCD₃, —CD₂CD₃, —CD₂CD₂H, —CD₂CDH₂, —CF₃, —CF₂H, —CFH₂, —CH₂CF₃, —CH₂CF₂H, —CH₂CFH₂, —CHFCH₃, —CHFCF₂H, —CHFCFH₂, —CHFCF₃, —CF₂CF₃, —CF₂CF₂H, or —CF₂CFH₂; or

an n-propyl group, an iso-propyl group, an n-butyl group, a sec-butyl group, an isobutyl group, a tert-butyl group, an n-pentyl group, a tert-pentyl group, a neopentyl group, an isopentyl group, a sec-pentyl group, a 3-pentyl group, a sec-isopentyl group, a phenyl group, a biphenyl group, or a naphthyl group, each unsubstituted or substituted with deuterium, —F, a C₁-C₁₀ alkyl group, a phenyl group, or any combination thereof, and

the remaining substituents may each be hydrogen.

In some embodiments, the heterocyclic compound may be represented by Formula 11-5.

In some embodiments, the heterocyclic compound may be Group HC2, and in some embodiments, the heterocyclic compound may be Compound 115001 or 115002.

The heterocyclic compound may include three of partial structure represented by

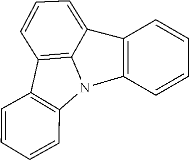 [SMILES: c1ccc2c(c1)c1cccc3c4ccccc4n2c13]

(hereinafter, referred to as an “ICz partial structure”). Accordingly, the heterocyclic compound may have improved multi-resonance characteristics, as compared with a compound including one or two of ICz partial structure. Thus, regardless of ΔE_{ST} value, a rate of reverse inter system crossing (RISC) may increase. Therefore, an organic light-emitting device including the heterocyclic compound may have improved efficiency.

In addition, as the heterocyclic compound may have improved multi-resonance characteristics, the heterocyclic compound may have a relatively small full width at half maximum (FWHM). Thus, an organic light-emitting device including the heterocyclic compound may have an improved colorimetric purity and/or conversion efficiency. For example, a FWHM of the heterocyclic compound may be smaller than 35 nm. In some embodiments, a FWHM of the heterocyclic compound may be 15 nm or less, but embodiments are not limited thereto.

The heterocyclic compound may satisfy Conditions 1 to 4:

$$Δ {E}_{ST} > Δ {E}_{ST 2} + Δ {E}_{TT}^{′} \qquad Condition 1$$

$$0 eV < Δ {E}_{ST 2} + Δ {E}_{TT}^{′} \leq 1. eV \qquad Condition 2$$

$$0 eV < Δ {E}_{TT}^{′} \leq 0.3 eV \qquad Condition 3$$

$$Δ {E}_{ST 2} > 0 eV \qquad Condition 4$$

wherein, in Conditions 1 to 4,

ΔE_{ST} indicates a difference between a lowest excited singlet energy level calculated in an S₁ equilibrium structure of the heterocyclic compound and a lowest excited triplet energy level calculated in a T1 equilibrium structure of the heterocyclic compound,

ΔE_{ST2} indicates a difference between a lowest excited singlet energy level calculated in an S₁ equilibrium structure of the heterocyclic compound and a second lowest excited triplet energy level calculated in a T₂ equilibrium structure of the heterocyclic compound, and

ΔE′_{TT} indicates a difference between a second lowest excited triplet energy level calculated in an T₂ equilibrium structure of the heterocyclic compound and a lowest excited triplet energy level calculated in a T₂ equilibrium structure of the heterocyclic compound.

The equilibrium structure is optimized according to the Turbomole program ([F. Furche et al. WIRESs: Comput. Mol. Sci. 4, 91-100 (2014)]) In some embodiments, structure optimization for T₁, T₂, and S1 states was performed according to a time-dependent density functional theory (DFT) by using the PBE0 function under Tamm-Dancoff approximation. To obtain normal modes, frequency calculation was performed, and a lowest energy structure was identified. By using the Q-Chem program ([Y. Shao et al. Mol. Phys. 113, 184-215 (2015)]), the nonadiabatic coupling between the excited triplet state and the T₁ state was calculated. In addition, the spin-orbit coupling between TDDFT states was calculated by using the Q-Chem program according to one-electron Breit-Pauli spin-orbit operator. The def2-SVP basis set was used for all atoms.

In some embodiments, the heterocyclic compound may satisfy Condition 3A:

$$0 eV < Δ {E}_{TT}^{′} \leq 0.15 eV \qquad Condition 3 A$$

wherein, in Condition 3A, ΔE′_{TT} may be understood by referring to the description of ΔE′_{TT} provided herein.

In general, compounds having a relatively small ΔE_{ST} value may emit thermal activated delayed fluorescence (TADF). However, even though the ΔE_{ST} value of the heterocyclic compound is relatively great, as the the heterocyclic compound may satisfy Conditions 1 to 4, the heterocyclic compound may emit TADF, and an organic light-emitting device including the heterocyclic compound may have improved efficiency.

Furthermore, by using the heterocyclic compound as a sensitizer, energy transferred to a triplet state may undergo RISC to a singlet state. Then, the singlet energy of the heterocyclic compound may be transferred to a dopant by Forster energy transfer. Thus, the organic light-emitting device may have improved efficiency and lifespan at the same time.

When heterocyclic compound serves as a dopant in an organic light-emitting device, the heterocyclic compound may emit blue light, e.g., blue light having a maximum emission wavelength of about 550 nm or less, for example, blue light having a maximum emission wavelength in a range of about 400 nm to about 500 nm, for example, about 420 nm to about 480 nm, or for example, 460 nm or less. The “maximum emission wavelength” as used herein refers to a wavelength of which the emission intensity is greatest. In other words, the “maximum emission wavelength” may be referred to as “peak emission wavelength”.

When the heterocyclic compound serves as a dopant in an organic light-emitting device, CIEy of the organic light-emitting device may be about 0.07 or less, for example, about 0.061 or less.

A method of synthesizing the heterocyclic compound may be apparent to one of ordinary skill in the art by referring to Synthesis Examples provided herein.

The heterocyclic compound represented by Formula 1 may be suitable for use in an organic layer of an organic light-emitting device, e.g., as a dopant in an emission layer of the organic layer. Thus, according to another aspect, there is provided an organic light-emitting device that may include: a first electrode; a second electrode; and an organic layer between the first electrode and the second electrode, wherein the organic layer includes an emission layer and at least one of the heterocyclic compound.

Since the organic light-emitting device has an organic layer including the heterocyclic compound, the organic light-emitting device may have a low driving voltage, high efficiency, high power, high quantum efficiency, long lifespan, low roll-off, and excellent color purity.

The heterocyclic compound may be used in a pair of electrodes of an organic light-emitting device. In some embodiments, the heterocyclic compound may be included in an emission layer. In this embodiment, the heterocyclic compound may serve as a dopant and the emission layer may further include a host (that is, an amount of the heterocyclic compound may be smaller than that of the host).

As used herein, the expression the “(organic layer) includes at least one of the heterocyclic compounds” may be construed as meaning that the “(organic layer) may include one heterocyclic compound of Formula 1-1 or 1-2 or two different heterocyclic compounds of Formula 1-1 or 1-2”.

For example, the organic layer may include Compound 115001 only as the heterocyclic compound. In this embodiment, Compound 115001 may be included in the emission layer of the organic light-emitting device. In some embodiments, the organic layer may include Compounds 115001 and 115002 as the heterocyclic compounds. In this embodiment, Compounds 115001 and 115002 may both be included in the same layer (for example, both Compounds 115001 and 115002 may be included in the emission layer).

The first electrode may be an anode, which is a hole injection electrode, and the second electrode may be a cathode, which is an electron injection electrode. In some embodiments, the first electrode may be a cathode, which is an electron injection electrode, and the second electrode may be an anode, which is a hole injection electrode.

For example, in the organic light-emitting device, the first electrode may be an anode, the second electrode may be a cathode, and the organic layer may further include a hole transport region between the first electrode and the emission layer and an electron transport region between the emission layer and the second electrode, wherein the hole transport region may include a hole injection layer, a hole transport layer, an electron blocking layer, or a combination thereof, and the electron transport region may include a hole blocking layer, an electron transport layer, an electron injection layer, a buffer layer, or a combination thereof.

The term “organic layer” as used herein refers to a single and/or a plurality of layers disposed between the first electrode and the second electrode in an organic light-emitting device. The “organic layer” may include not only organic compounds but also organometallic complexes including metals.

In some embodiments, the heterocyclic compound may emit fluorescence and/or delayed fluorescence. The emission layer that may emit fluorescence and/or delayed fluorescence is different from an emission layer that may emit phosphorescence.

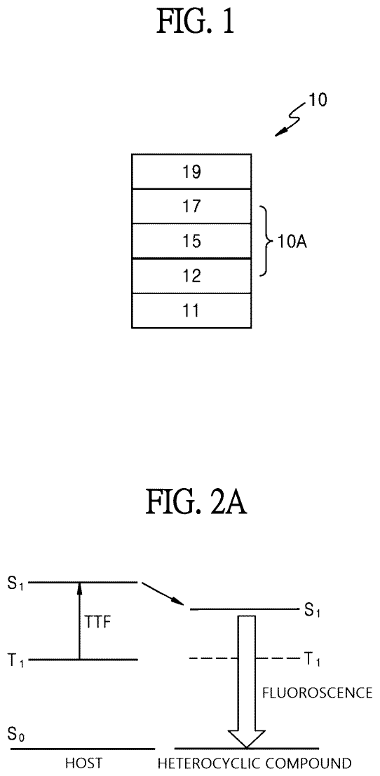

### Description of FIG. 1

FIG. 1 is a schematic view of an organic light-emitting device 10 according to an exemplary embodiment. Hereinafter a structure and a method of manufacturing the organic light-emitting device 10, according to an embodiment, will be described with reference to FIG. 1.

In FIG. 1, an organic light-emitting device 10 includes a first electrode 11, a second electrode 19 facing the first electrode 11, and an organic layer 10A between the first electrode 11 and the second electrode 19.

In FIG. 1, the organic layer 10A includes an emission layer 15, a hole transport region 12 is between the first electrode 11 and an emission layer 15, and an electron transport region 17 is between the emission layer 15 and the second electrode 19.

A substrate may be additionally disposed under the first electrode 11 or on the second electrode 19. The substrate may be a conventional substrate used in organic light-emitting devices, e.g., a glass substrate or a transparent plastic substrate, each having excellent mechanical strength, thermal stability, transparency, surface smoothness, ease of handling, and water repellency.

First electrode 11

The first electrode 11 may be produced by depositing or sputtering, onto the substrate, a material for forming the first electrode 11. The first electrode 11 may be an anode. The material for forming the first electrode 11 may be materials with a high work function for easy hole injection.

The first electrode 11 may be a reflective electrode, a semi-transmissive electrode, or a transmissive electrode. When the first electrode 11 is a transmissive electrode, a material for forming the first electrode 11 may be indium tin oxide (ITO), indium zinc oxide (IZO), tin oxide (SnO₂), zinc oxide (ZnO), or any combinations thereof, but embodiments are not limited thereto. In some embodiments, when the first electrode 11 is a semi-transmissive electrode or a reflective electrode, as a material for forming the first electrode 11, at least one of magnesium (Mg), silver (Ag), aluminum (Al), aluminum-lithium (Al—Li), calcium (Ca), magnesium-indium (Mg—In), magnesium-silver (Mg—Ag), or any combination thereof may be used, but embodiments are not limited thereto.

The first electrode 11 may have a single-layered structure or a multi-layered structure including a plurality of layers.

### Emission Layer 15

In some embodiments, the emission layer 15 may include the heterocyclic compound.

The thickness of the emission layer may be in a range of about 100 Å to about 1,000 Å, and in some embodiments, about 200 Å to about 600 Å. When the thickness of the emission layer is within any of these ranges, improved luminescence characteristics may be obtained without a substantial increase in driving voltage.

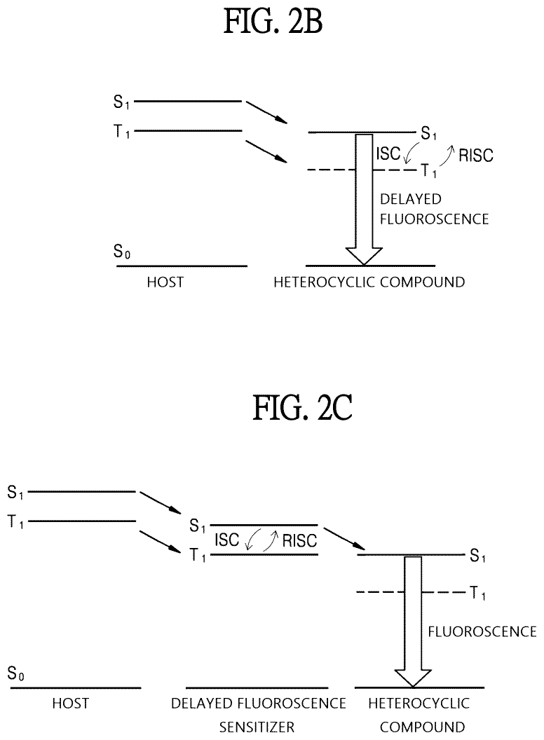

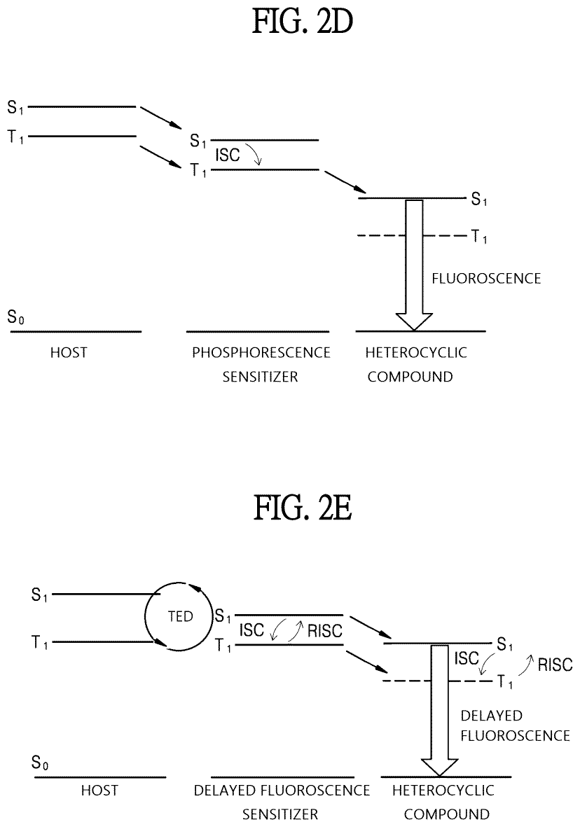

### First Embodiment—Descriptions of FIG. 2A

In the First Embodiment, the heterocyclic compound may be a fluorescence emitter. According to the First Embodiment, the emission layer may further include a host (hereinafter, referred to as ‘Host A’, and Host A may not be identical to the heterocyclic compound). Host A may be understood by referring to the description of the host material provided herein, but embodiments are not limited thereto. Host A may be a fluorescent host.

General energy transfer of the First Embodiment may be explained according to FIG. 2A.

Singlet excitons may be produced from Host A in the emission layer, and singlet excitons produced from Host A may be transferred to a fluorescence emitter through Förster energy transfer (FRET).

A ratio of singlet excitons produced from Host A may be 25%, and thus, 75% of triplet excitons produced from Host A may be fused to one another to be converted into singlet excitons. Thus, efficiency of the organic light-emitting device may be further improved. That is, efficiency of an organic light-emitting device may be further improved by using a triplet-triplet fusion mechanism.

According to the First Embodiment, a ratio of emission components emitted from the heterocyclic compound to the total emission components emitted from the emission layer may be about 80% or greater, for example, about 90% or greater. In some embodiments, a ratio of emission components emitted from the heterocyclic compound may be about 95% or greater to the total emission components emitted from the emission layer.

The heterocyclic compound may emit fluorescence, and the host may not emit light.

In the First Embodiment, when the emission layer further includes Host A, in addition to the heterocyclic compound, a content of the heterocyclic compound may be 50 parts by weight or less, e.g., 30 parts by weight or less, based on 100 parts by weight of the emission layer, and a content of Host A in the emission layer may be 50 parts by weight or greater, e.g., 70 parts by weight or greater, based on 100 parts by weight of the emission layer, but embodiments are not limited thereto.

In the First Embodiment, when the emission layer further includes Host A, in addition to the heterocyclic compound, Host A and the heterocyclic compound may satisfy Condition A:

$${E ( {H}_{A} )}_{S 1} > {E}_{S 1} \qquad Condition A$$

wherein, in Condition A,

E(H_{A})S₁ indicates a lowest excited singlet energy level of Host A, and

E_{S1} indicates a lowest excited singlet energy level of the heterocyclic compound.

Here, E(H_{A})_{S1} and E_{S1} may be evaluated by using Gaussian according to density functional theory (DFT) method (wherein structure optimization is performed at a degree of B3LYP, and 6-31G(d,p)).

### Second Embodiment—Descriptions of FIG. 2B

In the Second Embodiment, the heterocyclic compound may be a delayed fluorescence emitter. According to the Second Embodiment, the emission layer may further include a host (hereinafter, referred to as ‘Host B’, and Host B may not be identical to the heterocyclic compound). Host B may be understood by referring to the description of the host material provided herein, but embodiments are not limited thereto.

General energy transfer of the Second Embodiment may be explained according to FIG. 2B.

25% of singlet excitons produced from Host B in the emission layer may be transferred to a delayed fluorescence emitter through FRET. In addition, 75% of triplet excitons produced from Host B in the emission layer may be transferred to a delayed fluorescence emitter through Dexter energy transfer. Energy transferred to a triplet state of a delayed fluorescence emitter may undergo RISC to a singlet state. Accordingly, singlet excitons and triplet excitons produced from the emission layer may be transferred to the heterocyclic compound. Thus, the organic light-emitting device may have improved efficiency.

Thus, according to the Second Embodiment, a ratio of emission components emitted from the heterocyclic compound to the total emission components emitted from the emission layer may be about 80% or greater, for example, about 90% or greater. In some embodiments, a ratio of emission components emitted from the heterocyclic compound may be about 95% or greater to the total emission components emitted from the emission layer.

Here, the heterocyclic compound may emit fluorescence and/or delayed fluorescence, and the emission components of the heterocyclic compound may be a total of prompt emission components of the heterocyclic compound and delayed fluorescence components by RISC of the heterocyclic compound. In addition, Host B may not emit light.

In the Second Embodiment, when the emission layer further includes Host B, in addition to the heterocyclic compound, a content of the heterocyclic compound may be 50 parts by weight or less, e.g., 30 parts by weight or less, based on 100 parts by weight of the emission layer, and a content of Host B in the emission layer may be 50 parts by weight or greater, e.g., 70 parts by weight or greater, based on 100 parts by weight of the emission layer, but embodiments are not limited thereto.

In the Second Embodiment, when the emission layer further includes Host B, in addition to the heterocyclic compound, Host B and the heterocyclic compound may satisfy Condition B:

$${E ( {H}_{B} )}_{S 1} > {E}_{S 1} \qquad Condition B$$

wherein, in Condition B,

E(H_{B})_{S1} indicates a lowest excited singlet energy level of Host B, and

E_{S1} indicates a lowest excited singlet energy level of the heterocyclic compound.

Here, E(H_{B})_{S1} and E_{S1} may be evaluated by using Gaussian according to density functional theory (DFT) method (wherein structure optimization is performed at a degree of B3LYP, and 6-31G(d,p)).

Third Embodiment and Fourth Embodiment

### Third Embodiment—Descriptions of FIG. 2C

In the Third Embodiment, the heterocyclic compound may be used as a fluorescence emitter, and the emission layer may include a sensitizer, e.g., a delayed fluorescence sensitizer. In the Third Embodiment, the emission layer may further include a host (hereinafter, the host may be referred to as ‘Host C’, and Host C may not be identical to the heterocyclic compound and the sensitizer) and a sensitizer (hereinafter, the sensitizer may be referred to as ‘Sensitizer A’, and Sensitizer A may not be identical to Host C and the heterocyclic compound). Host C and Sensitizer A may respectively be understood by referring to the description of the host material and the sensitizer material provided herein, but embodiments are not limited thereto.

In the Third Embodiment, a ratio of emission components of the heterocyclic compound may be about 80% or greater, for example, about 90% or greater (or for example, about 95% or greater) to the total emission components emitted from the emission layer. For example, the heterocyclic compound may emit fluorescence. In addition, Host C and Sensitizer A may not each emit light.

General energy transfer of the Third Embodiment may be explained according to FIG. 2C.

Singlet and triplet excitons may be produced from Host C in the emission layer, and singlet and triplet excitons produced from Host C may be transferred to Sensitizer A and then to the heterocyclic compound through FRET. 25% of singlet excitons produced from Host C may be transferred to Sensitizer A through FRET, and energy of 75% of triplet excitons produced from Host C may be transferred to singlet and triplet states of Sensitizer A. Energy transferred to a triplet state of Sensitizer A may undergo RISC to a singlet state, and then, singlet energy of Sensitizer A may be transferred to the heterocyclic compound through FRET.

Accordingly, singlet excitons and triplet excitons produced from the emission layer may be transferred to the dopant. Thus, the organic light-emitting device may have improved efficiency. Further, energy loss of the organic light-emitting device may be significantly small. Thus, the organic light-emitting device may have improved lifespan characteristics.

In the Third Embodiment, when the emission layer further includes Host C and Sensitizer A, in addition to the heterocyclic compound, Host C and Sensitizer A may satisfy Condition C-1 and/or C-2:

$${S}_{1} ( {H}_{C} ) \geq {S}_{1} ( {S}_{A} ) \qquad Condition C - 1$$

$${S}_{1} ( {S}_{A} ) \geq {S}_{1} ( HC ) \qquad Condition C - 2$$

wherein, in Conditions C-1 and C-2,

S₁(H_{C}) indicates a lowest excited singlet energy level of Host C,

S₁(S_{A}) indicates a lowest excited singlet energy level of Sensitizer A, and

S₁(HC) indicates a lowest excited singlet energy level of the heterocyclic compound.

S₁(HC), S₁(S_{A}), and S₁(HC) may be evaluated according to the DFT method, wherein structure optimization is performed at a degree of B3LYP, and 6-31 G(d,p), for example, according to Gaussian according to DFT method.

When Host C, Sensitizer A, and the heterocyclic compound satisfy Condition C-1 and/or C-2, FRET from Sensitizer A to the heterocyclic compound may be facilitated, and accordingly, the organic light-emitting device may have improved luminescence efficiency.

### Fourth Embodiment—Descriptions of FIG. 2D

In the Fourth Embodiment, the heterocyclic compound may be used as a fluorescence emitter, and the emission layer may include a sensitizer, e.g., a phosphorescence sensitizer. In the Fourth Embodiment, the emission layer may further include a host (hereinafter, the host may be referred to as ‘Host D’, and Host D may not be identical to the heterocyclic compound and the sensitizer) and a sensitizer (hereinafter, the sensitizer may be referred to as ‘Sensitizer B’, and Sensitizer B may not be identical to Host D and the heterocyclic compound). Host D and Sensitizer B may respectively be understood by referring to the description of the host material and the sensitizer material provided herein, but embodiments are not limited thereto.

In the Fourth Embodiment, a ratio of emission components of the heterocyclic compound may be about 80% or greater, for example, about 90% or greater (or for example, about 95% or greater) to the total emission components emitted from the emission layer. For example, the heterocyclic compound may emit fluorescence. In addition, Host D and Sensitizer B may not each emit light.

General energy transfer of the Fourth Embodiment may be explained according to FIG. 2D. [00132]75% of triplet excitons produced from Host D in the emission layer may be transferred to Sensitizer B through Dexter energy transfer, and the energy of 25% of singlet excitons produced from Host D may be transferred to singlet and triplet states of Sensitizer B. Energy transferred to a singlet state of Sensitizer B may undergo ISC to a triplet state, and then, triplet energy of Sensitizer B may be transferred to the heterocyclic compound through FRET.

Accordingly, singlet excitons and triplet excitons produced from the emission layer may be transferred to the dopant. Thus, the organic light-emitting device may have improved efficiency. Further, energy loss of the organic light-emitting device may be significantly small. Thus, the organic light-emitting device may have improved lifespan characteristics.

In the Third Embodiment, when the emission layer further includes Host D and Sensitizer B, in addition to the heterocyclic compound, Host D and Sensitizer B may satisfy Condition D-1 and/or D-2:

$${T}_{1} ( {H}_{D} ) \geq {T}_{1} ( {S}_{B} ) \qquad Condition D - 1$$

$${T}_{1} ( {S}_{B} ) \geq {S}_{1} ( HC ) \qquad Condition D - 2$$

wherein, in Conditions D-1 and D-2,
T₁(H_{D}) indicates a lowest excited triplet energy level of Host D,
T₁(S_{B}) indicates a lowest excited triplet energy level of Sensitizer B, and
S₁(HC) indicates a lowest excited singlet energy level of the heterocyclic compound.

T₁(H_{D}), T₁(S_{B}), and S₁(HC) may be evaluated according to the DFT method, wherein structure optimization is performed at a degree of B3LYP, and 6-31 G(d,p), for example, according to Gaussian according to DFT method.

When Host D, Sensitizer B, and the heterocyclic compound satisfy Condition D-1 and/or D-2, FRET from Sensitizer B to the heterocyclic compound may be facilitated, and accordingly, the organic light-emitting device may have improved luminescence efficiency.

In the Third Embodiment and the Fourth Embodiment, a content of the sensitizer in the emission layer may be in a range of about 5 percent by weight (wt %) to about 50 wt %, or for example, about 10 wt % to about 30 wt %. When the content is within this range, energy transfer in the emission layer may be effectively occurred. Thus, the organic light-emitting device may have high efficiency and long lifespan.

In the Third Embodiment and the Fourth Embodiment, a content of the heterocyclic compound in the emission layer may be in a range of about 0.01 wt % to about 15 wt %, or for example, about 0.05 wt % to about 3 wt %, but embodiments are not limited thereto.

In the Third Embodiment and the Fourth Embodiment, the heterocyclic compound may further satisfy Condition 5:

$$0 µs < {T}_{decay} ( HC ) < 5 µs \qquad Condition 5$$

wherein, in Condition 5, T_{decay}(HC) indicates a decay time of the heterocyclic compound.

The decay time of the heterocyclic compound was measured from a time-resolved photoluminescence (TRPL) spectrum at room temperature of a film (hereinafter, referred to as “Film (HC)”) having a thickness of about 40 nm formed by vacuum-depositing the host and the heterocyclic compound included in the emission layer on a quartz substrate at a weight ratio of 90:10 at a vacuum pressure of 10⁻⁷ torr.

### Fifth Embodiment—Descriptions of FIG. 2E

In the Fifth Embodiment, the heterocyclic compound may be used as a delayed fluorescence emitter, and the emission layer may include a sensitizer, e.g., a delayed fluorescence sensitizer. In the Fifth Embodiment, the emission layer may further include a host (hereinafter, the host may be referred to as ‘Host E’, and Host E may not be identical to the heterocyclic compound and the sensitizer) and a sensitizer (hereinafter, the sensitizer may be referred to as ‘Sensitizer C’, and Sensitizer C may not be identical to Host E and the heterocyclic compound). Host E and Sensitizer C may respectively be understood by referring to the description of the host material and the sensitizer material provided herein, but embodiments are not limited thereto.

In the Fifth Embodiment, a ratio of emission components of the heterocyclic compound may be about 80% or greater, for example, about 90% or greater (or for example, about 95% or greater) to the total emission components emitted from the emission layer. In some embodiments, the heterocyclic compound may emit fluorescence and/or delayed fluorescence. In addition, Host E and Sensitizer C may not each emit light.

Here, the heterocyclic compound may emit fluorescence and/or delayed fluorescence, and the emission components of the heterocyclic compound may be a total of prompt emission components of the heterocyclic compound and delayed fluorescence components by RISC of the heterocyclic compound.

General energy transfer of the Fifth Embodiment may be explained according to FIG. 2E.

25% of singlet excitons produced from Host E in the emission layer may be transferred to a singlet state of Sensitizer C through FRET, and the energy of 75% of triplet excitons produced from Host E may be transferred to a triplet state of Sensitizer C, and then singlet energy of Sensitizer C may be transferred to the heterocyclic compound through FRET. Subsequently, the triplet energy of Sensitizer C may be transferred to the heterocyclic compound through Dexter energy transfer. Energy transferred to a triplet state of Sensitizer C may undergo RISC to a singlet state. Further, in a case of Sensitizer C, energy of triplet excitons produced from Sensitizer C may undergo reverse transfer to Host E and then to the heterocyclic compound, thus emitting by reverse intersystem transfer.

Accordingly, singlet excitons and triplet excitons produced from the emission layer may be transferred to the dopant. Thus, the organic light-emitting device may have improved efficiency. Further, energy loss of the organic light-emitting device may be significantly small. Thus, the organic light-emitting device may have improved lifespan characteristics.

In the Fifth Embodiment, when the emission layer further includes Host E and Sensitizer C, in addition to the heterocyclic compound, Host E and Sensitizer C may satisfy Condition E-1, E-2, and/or E-3:

$${S}_{1} ( {H}_{E} ) \geq {S}_{1} ( {S}_{C} ) \qquad Condition E - 1$$

$${S}_{1} ( {S}_{C} ) \geq {S}_{1} ( HC ) \qquad Condition E - 2$$

wherein, in Conditions E-1, E-2, and E-3,

S₁(H_{E}) indicates a lowest excited singlet energy level of Host E,

S₁(S_{C}) indicates a lowest excited singlet energy level of Sensitizer C,

S₁(HC) indicates a lowest excited singlet energy level of the heterocyclic compound,

T₁(S_{C}) indicates a lowest excited triplet energy level of Sensitizer C, and

T₁(HC) indicates a lowest excited triplet energy level of the heterocyclic compound.

S₁(H_{E}), S₁(S_{C}), S₁(HC), T₁(S_{C}), and T₁(HC) may be evaluated according to the DFT method, wherein structure optimization is performed at a degree of B3LYP, and 6-31G(d,p), for example, according to Gaussian according to DFT method.

When Host E, Sensitizer C, and the heterocyclic compound satisfy Condition E-1, E-2, and/or E-3, Dexter transfer FRET from Sensitizer C to the heterocyclic compound may be facilitated, and accordingly, the organic light-emitting device may have improved luminescence efficiency.

In the Fifth Embodiment, a content of Sensitizer C in the emission layer may be in a range of about 5 wt % to about 50 wt %, or for example, about 10 wt % to about 30 wt %. When the content is within this range, energy transfer in the emission layer may occur effectively. Thus, the organic light-emitting device may have high efficiency and long lifespan.

In the Fifth Embodiment, a content of the heterocyclic compound in the emission layer may be in a range of about 0.01 wt % to about 15 wt %, or for example, about 0.05 wt % to about 3 wt %, but embodiments are not limited thereto.

### Host in Emission Layer 15

The host may not include a metal atom.

In an embodiment, the host may consist of one type of host. When the host consists of one type of host, the one type of host may be a bipolar host, an electron transporting host, or a hole transporting host described herein.

In one or more embodiments, the host may be a mixture of two or more types of hosts. In some embodiments, the host may be a mixture of an electron transporting host and a hole transporting host, a mixture of two different types of electron transporting hosts or a mixture of two different types of hole transporting hosts. The electron transporting host and the hole transporting host may respectively be understood by referring to the descriptions of the electron transporting host and the hole transporting host provided herein.

In an embodiment, the host may include an electron transporting host including at least one electron transporting moiety and a hole transporting host not including an electron transporting moiety.

The electron transporting moiety may be a cyano group, a π electron-depleted nitrogen-containing cyclic group, and a group represented by one of following Formulae:

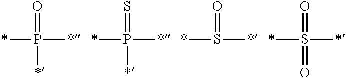

wherein, in the Formulae above, *, *′, and *″ may each indicate a binding site to an adjacent atom.

In an embodiment, an electron transporting host in the emission layer 15 may include at least one of a cyano group, a π electron-depleted nitrogen-containing cyclic group, or a combination thereof.

In one or more embodiments, the electron transporting host in the emission layer 15 may include at least one cyano group.

In one or more embodiments, an electron transporting host in the emission layer 15 may include a cyano group, at least one π electron-depleted nitrogen-containing cyclic group, or a combination thereof.

In one or more embodiments, the host may include an electron transporting host and a hole transporting host, the electron transporting host may include at least one rr electron-depleted nitrogen-free cyclic group and at least one electron transporting moiety, and the hole transporting host may include at least one π electron-depleted nitrogen-free cyclic group and may not include an electron transporting moiety.

The term “π electron-depleted nitrogen-containing cyclic group” as used herein refers to a cyclic group having at least one *—N═*′ moiety. Examples thereof may include: an imidazole group, a pyrazole group, a thiazole group, an isothiazole group, an oxazole group, an isoxazole group, a pyridine group, a pyrazine group, a pyridazine group, a pyrimidine group, an indazole group, a purine group, a quinoline group, an isoquinoline group, a benzoquinoline group, a phthalazine group, a naphthyridine group, a quinoxaline group, a quinazoline group, a cinnoline group, a phenanthridine group, an acridine group, a phenanthroline group, a phenazine group, a benzimidazole group, an isobenzothiazole group, a benzoxazole group, an isobenzoxazole group, a triazole group, a tetrazole group, an oxadiazole group, a triazine group, a thiadiazole group, an imidazopyridine group, an imidazopyrimidine group, and an azacarbazole group; and a condensed ring of at least two π electron-depleted nitrogen-containing cyclic groups.

The π electron-depleted nitrogen-free cyclic group may be a benzene group, a heptalene group, an indene group, a naphthalene group, an azulene group, an indacene group, an acenaphthylene group, a fluorene group, a spiro-bifluorene group, a benzofluorene group, a dibenzofluorene group, a phenalene group, a phenanthrene group, an anthracene group, a fluoranthene group, a triphenylene group, a pyrene group, a chrysene group, a naphthacene group, a picene group, a perylene group, a pentacene group, a hexacene group, a pentacene group, a rubicene group, a coronene group, an ovalene group, a pyrrole group, an isoindole group, an indole group, a furan group, a thiophene group, a benzofuran group, a benzothiophene group, a benzocarbazole group, a dibenzocarbazole group, a dibenzofuran group, a dibenzothiophene group, a dibenzothiophene sulfone group, a carbazole group, a dibenzosilole group, an indenocarbazole group, an indolocarbazole group, a benzofurocarbazole group, a benzothienocarbazole group, and a triindolobenzene group; and a condensed ring of at least two π electron-depleted nitrogen-free cyclic group, but embodiments are not limited thereto.

In some embodiments, the electron transporting host may be one of the Compounds represented by Formula E-1, and

the hole transporting host may be one of the Compounds represented by Formula H-1, but embodiments are not limited thereto:

[Ar₃₀₁]_{xb11}—[(L₃₀₁)_{xb1}—R₃₀₁]_{xb21} Formula E-1

wherein, in Formula E-1,

Ar₃₀₁ may be a substituted or unsubstituted C₅-C₆₀ carbocyclic group or a substituted or unsubstituted C₁-C₆₀ heterocyclic group,

xb11 may be 1, 2, or 3,

L₃₀₁ may each independently be a single bond, a group represented by one of following Formulae, a substituted or unsubstituted C₅-C₆₀ carbocyclic group, or a substituted or unsubstituted C₁-C₆₀ heterocyclic group, wherein in the following Formulae, and *″ may each indicate a binding site to an adjacent atom,

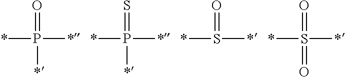

xb1 may be an integer from 1 to 5,

R₃₀₁ may be hydrogen, deuterium, —F, —Cl, —Br, —I, a hydroxyl group, a cyano group, a nitro group, an amidino group, a hydrazino group, a hydrazono group, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent aromatic condensed polycyclic group, a substituted or unsubstituted monovalent aromatic condensed heteropolycyclic group, a substituted or unsubstituted monovalent non-aromatic condensed polycyclic group, a substituted or unsubstituted monovalent non-aromatic condensed heteropolycyclic group, —Si(Q₃₀₁)(Q₃₀₂)(Q₃₀₃), —N(Q₃₀₁)(Q₃₀₂), —B(Q₃₀₁)(Q₃₀₂), —C(═O)(Q₃₀₁), —S(═O)₂(Q₃₀₁), —S(═O)(Q₃₀₁), —P(═O)(Q₃₀₁)(Q₃₀₂), or —P(═S)(Q₃₀₁)(Q₃₀₂),

xb21 may be an integer from 1 to 5,

Q₃₀₁ to Q₃₀₃ may each independently be a C₁-C₁₀ alkyl group, a C₁-C₁₀ alkoxy group, a phenyl group, a biphenyl group, a terphenyl group, or a naphthyl group, and

at least one of Conditions H-1 to H-3 may be satisfied:

### Condition H-1

wherein, at least one of Ar₃₀₁, L₃₀₁, and R₃₀₁ in Formula E-1 may each independently include a π electron-depleted nitrogen-containing cyclic group,

### Condition H-2

wherein, L₃₀₁ in Formula E-1 may be a group represented by one of the following Formulae, and

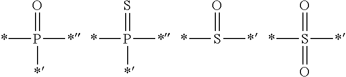

### Condition H-3

wherein, R₃₀₁ in Formula E-1 may be a cyano group, —S(═O)₂(Q₃₀₁), —S(═O)(Q₃₀₁), —P(═O)(Q₃₀₁)(Q₃₀₂), or —P(═S)(Q₃₀₁)(Q₃₀₂).

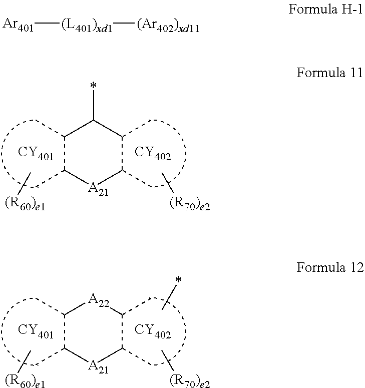

wherein, in Formulae H-1, 11, and 12,

L₄₀₁ may be:

a single bond; or

a benzene group, a heptalene group, an indene group, a naphthalene group, an azulene group, an indacene group, an acenaphthylene group, a fluorene group, a spiro-bifluorene group, a benzofluorene group, a dibenzofluorene group, a phenalene group, a phenanthrene group, an anthracene group, a fluoranthene group, a triphenylene group, a pyrene group, a chrysene group, a naphthacene group, a picene group, a perylene group, a pentacene group, a hexacene group, a pentacene group, a rubicene group, a coronene group, an ovalene group, a pyrrole group, an isoindole group, an indole group, a furan group, a thiophene group, a benzofuran group, a benzothiophene group, a benzocarbazole group, a dibenzocarbazole group, a dibenzofuran group, a dibenzothiophene group, a dibenzothiophene sulfone group, a carbazole group, a dibenzosilole group, an indenocarbazole group, an indolocarbazole group, a benzofurocarbazole group, a benzothienocarbazole group, or a triindolobenzene group, each unsubstituted or substituted with at least one of deuterium, a C₁-C₁₀ alkyl group, a C₁-C₁₀ alkoxy group, a phenyl group, a naphthyl group, a fluorenyl group, a carbazolyl group, a dibenzofuranyl group, a dibenzothiophenyl group, a triphenylenyl group, a biphenyl group, a terphenyl group, a tetraphenyl group, —Si(Q₄₀₁)(Q₄₀₂)(Q₄₀₃), or any combination thereof.

xd1 may be an integer from 1 to 10, and when xd1 is 2 or greater, at least two L₄₀₁(s) may be identical to or different from each other,

Ar₄₀₁ may be a group represented by Formulae 11 or 12,

Ar₄₀₂ may be:

a group represented by Formulae 11 or 12, a phenyl group, a naphthyl group, a fluorenyl group, a carbazolyl group, a dibenzofuranyl group, a dibenzothiophenyl group, a biphenyl group, a terphenyl group, or a triphenylenyl group; or

a phenyl group, a naphthyl group, a fluorenyl group, a carbazolyl group, a dibenzofuranyl group, a dibenzothiophenyl group, a biphenyl group, a terphenyl group, or a triphenylenyl group, each unsubstituted or substituted with at least one deuterium, a hydroxyl group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a phenyl group, a naphthyl group, a fluorenyl group, a carbazolyl group, a dibenzofuranyl group, a dibenzothiophenyl group, a biphenyl group, a terphenyl group, a triphenylenyl group, or any combination thereof,

CY₄₀₁ and CY₄₀₂ may each independently be a benzene group, a naphthalene group, a fluorene group, a carbazole group, a benzocarbazole group, an indolocarbazole group, a dibenzofuran group, a dibenzothiophene group, a dibenzosilole group, a benzonaphthofuran group, a benzonapthothiophene group, or a benzonaphthosilole group,

A₂₁ may be a single bond, O, S, N(R₅₁), C(R₅₁)(R₅₂), or Si(R₅₁)(R₅₂),

A₂₂ may be a single bond, O, S, N(R₅₃), C(R₅₃)(R₅₄), or Si(R₅₃)(R₅₄),

at least one of A₂₁, A₂₂, or any combination thereof in Formula 12 may not be a single bond,

R₅₁ to R₅₄ and R₆₀ to R₇₀ may each independently be:

hydrogen, deuterium, a hydroxyl group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, or a C₁-C₂₀ alkoxy group;

a C₁-C₂₀ alkyl group or a C₁-C₂₀ alkoxy group, each substituted with at least one deuterium, a hydroxyl group, an amino group, an amidino group, a hydrazine group, a hydrazone group, a carboxylic acid group or a salt thereof, a sulfonic acid group or a salt thereof, a phosphoric acid group or a salt thereof, a phenyl group, a naphthyl group, a fluorenyl group, a carbazolyl group, a dibenzofuranyl group, a dibenzothiophenyl group, or any combination thereof;

a π electron-depleted nitrogen-free cyclic group (e.g., a phenyl group, a naphthyl group, a fluorenyl group, a carbazolyl group, a dibenzofuranyl group, a dibenzothiophenyl group, a biphenyl group, a terphenyl group, and a triphenylenyl group);

a π electron-depleted nitrogen-free cyclic group (e.g., a phenyl group, a naphthyl group, a fluorenyl group, a carbazolyl group, a dibenzofuranyl group, a dibenzothiophenyl group, a biphenyl group, a terphenyl group, and a triphenylenyl group) substituted with at least one deuterium, a hydroxyl group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a phenyl group, a naphthyl group, a fluorenyl group, a carbazolyl group, a dibenzofuranyl group, a dibenzothiophenyl group, a biphenyl group, or any combination thereof,

—Si(Q₄₀₄)(Q₄₀₅)(Q₄₀₆),

e1 and e2 may each independently be an integer from 0 to 10,

wherein Q₄₀₁ to Q₄₀₆ may each independently be hydrogen, deuterium, a hydroxyl group, an amino group, an amidino group, a hydrazine group, a hydrazone group, a carboxylic acid group or a salt thereof, a sulfonic acid group or a salt thereof, a phosphoric acid group or a salt thereof, a phenyl group, a naphthyl group, a fluorenyl group, a carbazolyl group, a dibenzofuranyl group, a dibenzothiophenyl group, a biphenyl group, a terphenyl group, or a triphenylenyl group, and * indicates a binding site to an adjacent atom.

In some embodiments, in Formula E-1, Ar₃₀₁ and L₃₀₁ may each independently be a benzene group, a naphthalene group, a fluorene group, a spiro-bifluorene group, a benzofluorene group, a dibenzofluorene group, a phenalene group, a phenanthrene group, an anthracene group, a fluoranthene group, a triphenylene group, a pyrene group, a chrysene group, a naphthacene group, a picene group, a perylene group, a pentaphene group, an indenoanthracene group, a dibenzofuran group, a dibenzothiophene group, an imidazole group, a pyrazole group, a thiazole group, an isothiazole group, an oxazole group, an isoxazole group, a pyridine group, a pyrazine group, a pyridazine group, a pyrimidine group, an indazole group, a purine group, a quinoline group, an isoquinoline group, a benzoquinoline group, a phthalazine group, a naphthyridine group, a quinoxaline group, a quinazoline group, a cinnoline group, a phenanthridine group, an acridine group, a phenanthroline group, a phenazine group, a benzimidazole group, an isobenzothiazole group, a benzoxazole group, an isobenzoxazole group, a triazole group, a tetrazole group, an oxadiazole group, a triazine group, a thiadiazole group, an imidazopyridine group, an imidazopyrimidine group, or an azacarbazole group, each unsubstituted or substituted with at least one deuterium, —F, —Cl, —Br, —I, a hydroxyl group, a cyano group, a nitro group, an amidino group, a hydrazino group, a hydrazono group, a C₁-C₂₀ alkyl group, a C₁-C₂₀ alkoxy group, a phenyl group, a biphenyl group, a terphenyl group, a naphthyl group, a cyano group-containing phenyl group, a cyano group-containing biphenyl group, a cyano group-containing terphenyl group, a cyano group-containing naphthyl group, a pyridinyl group, a phenylpyridinyl group, a diphenylpyridinyl group, a biphenylpyridinyl group, a di(biphenyl)pyridinyl group, a pyrazinyl group, a phenylpyrazinyl group, a diphenylpyrazinyl group, a biphenylpyrazinyl group, a di(biphenyl)pyrazinyl group, a pyridazinyl group, a phenylpyridazinyl group, a diphenylpyridazinyl group, a biphenylpyridazinyl group, a di(biphenyl)pyridazinyl group, a pyrimidinyl group, a phenylpyrimidinyl group, a diphenylpyrimidinyl group, a biphenylpyrimidinyl group, a di(biphenyl)pyrimidinyl group, a triazinyl group, a phenyltriazinyl group, a diphenyltriazinyl group, a biphenyltriazinyl group, a di(biphenyl)triazinyl group, —Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), —P(═O)(Q₃₁)(Q₃₂), or any combination thereof, at least one of L₃₀₁ in the number of xb1 may each independently be an imidazole group, a pyrazole group, a thiazole group, an isothiazole group, an oxazole group, an isoxazole group, a pyridine group, a pyrazine group, a pyridazine group, a pyrimidine group, an indazole group, a purine group, a quinoline group, an isoquinoline group, a benzoquinoline group, a phthalazine group, a naphthyridine group, a quinoxaline group, a quinazoline group, a cinnoline group, a phenanthridine group, an acridine group, a phenanthroline group, a phenazine group, a benzimidazole group, an isobenzothiazole group, a benzoxazole group, an isobenzoxazole group, a triazole group, a tetrazole group, an oxadiazole group, a triazine group, a thiadiazole group, an imidazopyridine group, an imidazopyrimidine group, or an azacarbazole group, each unsubstituted or substituted with at least one deuterium, —F, —Cl, —Br, —I, a hydroxyl group, a cyano group, a nitro group, an amidino group, a hydrazino group, a hydrazono group, a C₁-C₂₀ alkyl group, a C₁-C₂₀ alkoxy group, a phenyl group, a biphenyl group, a terphenyl group, a naphthyl group, a cyano group-containing phenyl group, a cyano group-containing biphenyl group, a cyano group-containing terphenyl group, a cyano group-containing naphthyl group, a pyridinyl group, a phenylpyridinyl group, a diphenylpyridinyl group, a biphenylpyridinyl group, a di(biphenyl)pyridinyl group, a pyrazinyl group, a phenylpyrazinyl group, a diphenylpyrazinyl group, a biphenylpyrazinyl group, a di(biphenyl)pyrazinyl group, a pyridazinyl group, a phenylpyridazinyl group, a diphenylpyridazinyl group, a biphenylpyridazinyl group, a di(biphenyl)pyridazinyl group, a pyrimidinyl group, a phenylpyrimidinyl group, a diphenylpyrimidinyl group, a biphenylpyrimidinyl group, a di(biphenyl)pyrimidinyl group, a triazinyl group, a phenyltriazinyl group, a diphenyltriazinyl group, a biphenyltriazinyl group, a di(biphenyl)triazinyl group, —Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), and R₃₀₁ may be hydrogen, deuterium, —F, —Cl, —Br, —I, a hydroxyl group, a cyano group, a nitro group, an amidino group, a hydrazino group, a hydrazono group, a C₁-C₂₀ alkyl group, a C₁-C₂₀ alkoxy group, a phenyl group, a biphenyl group, a terphenyl group, a tetraphenyl group, a naphthyl group, a cyano group-containing phenyl group, a cyano group-containing biphenyl group, a cyano group-containing terphenyl group, a cyano group-containing a tetraphenyl group, a cyano group-containing naphthyl group, a pyridinyl group, a phenylpyridinyl group, a diphenylpyridinyl group, a biphenylpyridinyl group, a di(biphenyl)pyridinyl group, a pyrazinyl group, a phenylpyrazinyl group, a diphenylpyrazinyl group, a biphenylpyrazinyl group, a di(biphenyl)pyrazinyl group, a pyridazinyl group, a phenylpyridazinyl group, a diphenylpyridazinyl group, a biphenylpyridazinyl group, a di(biphenyl)pyridazinyl group, a pyrimidinyl group, a phenylpyrimidinyl group, a diphenylpyrimidinyl group, a biphenylpyrimidinyl group, a di(biphenyl)pyrimidinyl group, a triazinyl group, a phenyltriazinyl group, a diphenyltriazinyl group, a biphenyltriazinyl group, a di(biphenyl)triazinyl group, —Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), wherein Q₃₁ to Q₃₃ may each independently be a C₁-C₁₀ alkyl group, a C₁-C₁₀ alkoxy group, a phenyl group, a biphenyl group, a terphenyl group, or a naphthyl group, but embodiments are not limited thereto.

In some embodiments, Ar₃₀₁ may be a benzene group, a naphthalene group, a fluorene group, a spiro-bifluorene group, a benzofluorene group, a dibenzofluorene group, a phenalene group, a phenanthrene group, an anthracene group, a fluoranthene group, a triphenylene group, a pyrene group, a chrysene group, a naphthacene group, a picene group, a perylene group, a pentaphene group, an indenoanthracene group, a dibenzofuran group, or a dibenzothiophene group, each unsubstituted or substituted with at least one deuterium, —F, —Cl, —Br, —I, a hydroxyl group, a cyano group, a nitro group, an amidino group, a hydrazino group, a hydrazono group, a C₁-C₂₀ alkyl group, a C₁-C₂₀ alkoxy group, a phenyl group, a biphenyl group, a terphenyl group, a naphthyl group, a cyano group-containing phenyl group, a cyano group-containing biphenyl group, a cyano group-containing terphenyl group, a cyano group-containing naphthyl group, a pyridinyl group, a phenylpyridinyl group, a diphenylpyridinyl group, a biphenylpyridinyl group, a di(biphenyl)pyridinyl group, a pyrazinyl group, a phenylpyrazinyl group, a diphenylpyrazinyl group, a biphenylpyrazinyl group, a di(biphenyl)pyrazinyl group, a pyridazinyl group, a phenylpyridazinyl group, a diphenylpyridazinyl group, a biphenylpyridazinyl group, a di(biphenyl)pyridazinyl group, a pyrimidinyl group, a phenylpyrimidinyl group, a diphenylpyrimidinyl group, a biphenylpyrimidinyl group, a di(biphenyl)pyrimidinyl group, a triazinyl group, a phenyltriazinyl group, a diphenyltriazinyl group, a biphenyltriazinyl group, a di(biphenyl)triazinyl group, —Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁) or —P(═O)(Q₃₁)(Q₃₂); or

a group represented by one of Formulae 5-1 to 5-3 and 6-1 to 6-33, and L₃₀₁ may be a group represented by one of Formulae 5-1 to 5-3 and 6-1 to 6-33:

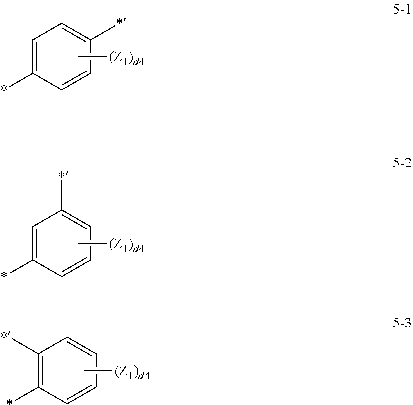

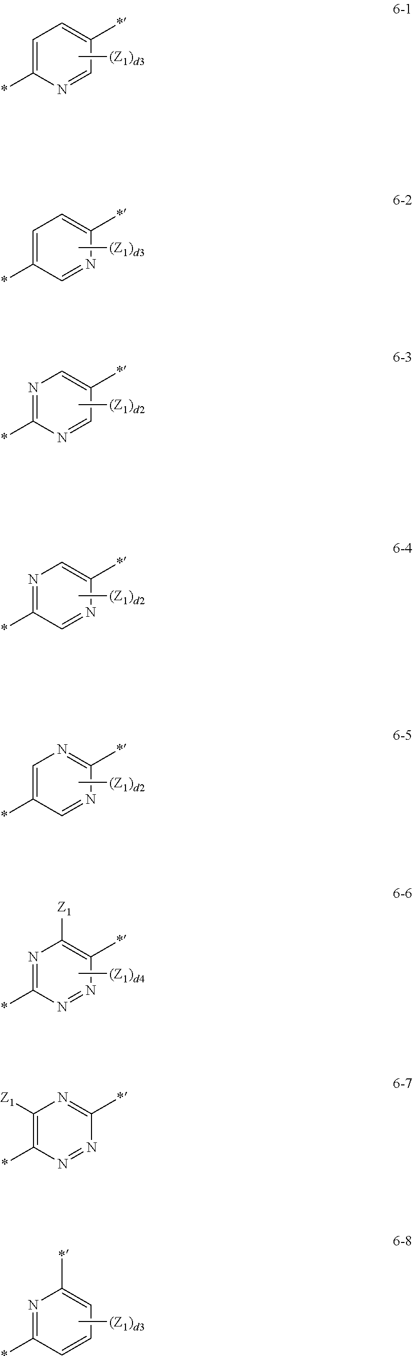

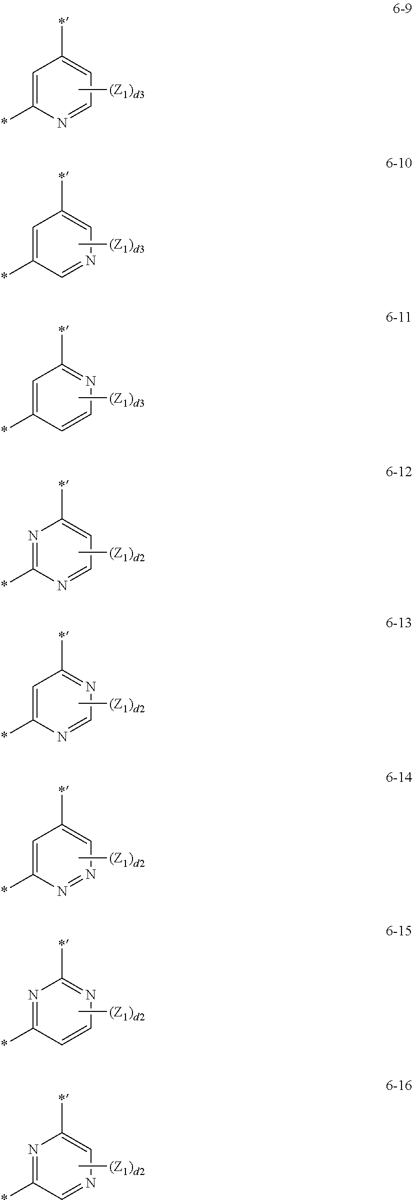

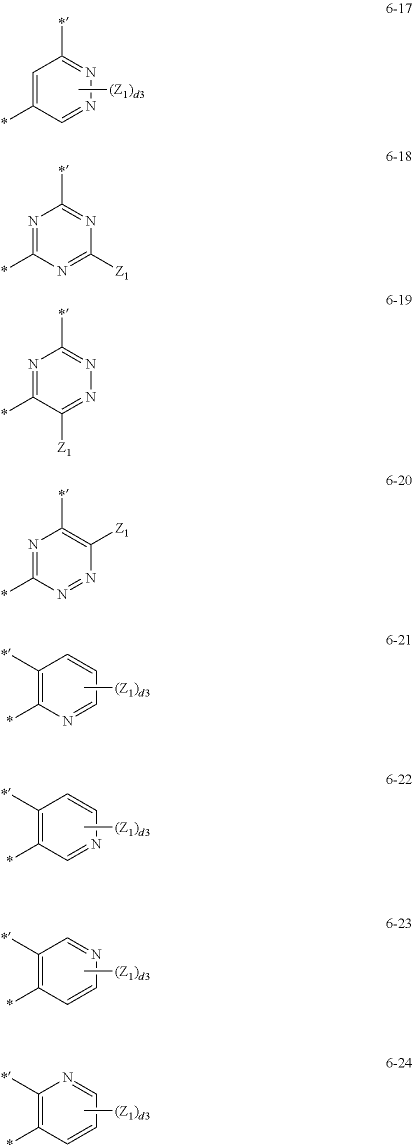

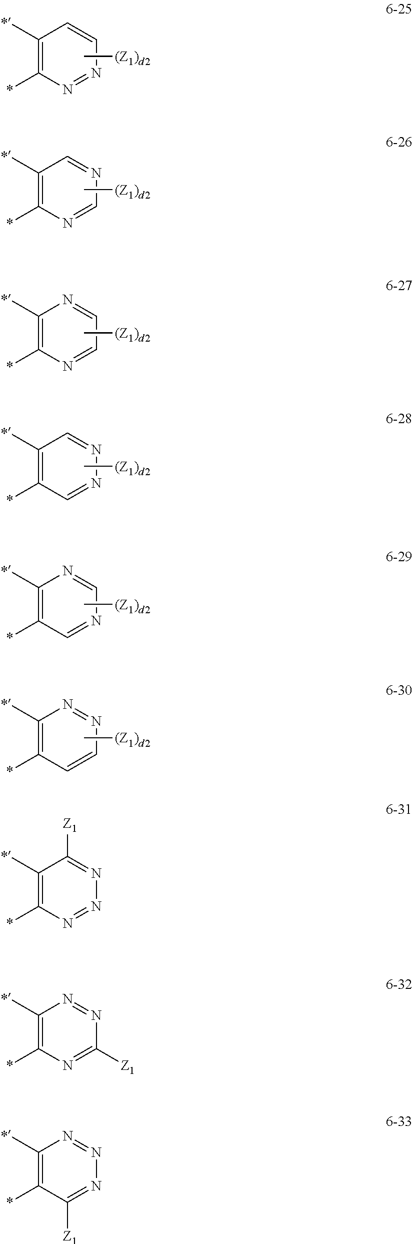

wherein, in Formulae 5-1 to 5-3 and 6-1 to 6-33,

Z₁ may be hydrogen, deuterium, —F, —Cl, —Br, —I, a hydroxyl group, a cyano group, a nitro group, an amidino group, a hydrazino group, a hydrazono group, a C₁-C₂₀ alkyl group, a C₁-C₂₀ alkoxy group, a phenyl group, a biphenyl group, a terphenyl group, a naphthyl group, a cyano group-containing phenyl group, a cyano group-containing biphenyl group, a cyano group-containing terphenyl group, a cyano group-containing naphthyl group, a pyridinyl group, a phenylpyridinyl group, a diphenylpyridinyl group, a biphenylpyridinyl group, a di(biphenyl)pyridinyl group, a pyrazinyl group, a phenylpyrazinyl group, a diphenylpyrazinyl group, a biphenylpyrazinyl group, a di(biphenyl)pyrazinyl group, a pyridazinyl group, a phenylpyridazinyl group, a diphenylpyridazinyl group, a biphenylpyridazinyl group, a di(biphenyl)pyridazinyl group, a pyrimidinyl group, a phenylpyrimidinyl group, a diphenylpyrimidinyl group, a biphenylpyrimidinyl group, a di(biphenyl)pyrimidinyl group, a triazinyl group, a phenyltriazinyl group, a diphenyltriazinyl group, a biphenyltriazinyl group, a di(biphenyl)triazinyl group, —Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂),

d4 may be 0, 1, 2, 3, or 4,

d3 may be 0, 1, 2, or 3,

d2 may be 0, 1, or 2, and

* and *′ each indicate a binding site to an adjacent atom.

Q₃₁ to Q₃₃ may respectively be understood by referring to the descriptions of Q₃₁ to Q₃₃ provided herein.

In one or more embodiments, L₃₀₁ may be groups represented by Formulae 5-2, 5-3, and 6-8 to 6-33.

In one or more embodiments, R₃₀₁ may be a cyano group or a group represented by Formulae 7-1 to 7-18, at least one Ar₄₀₂ in the number of xd11 may be a group represented by Formulae 7-1 to 7-18, but embodiments are not limited thereto:

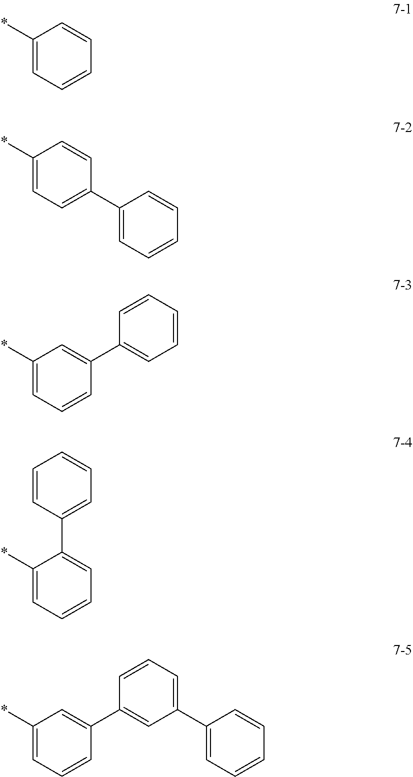

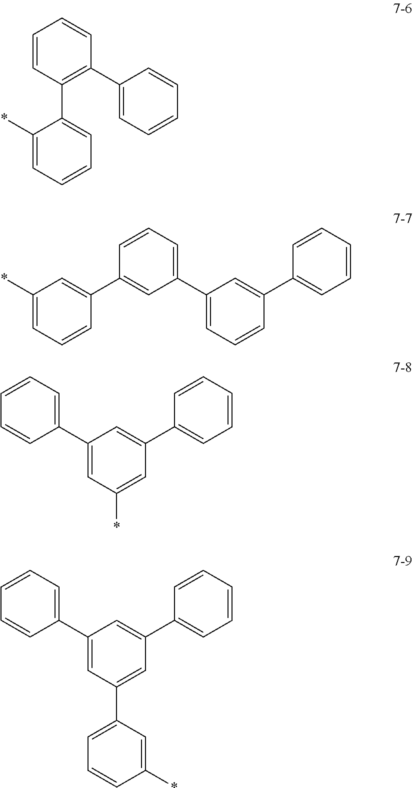

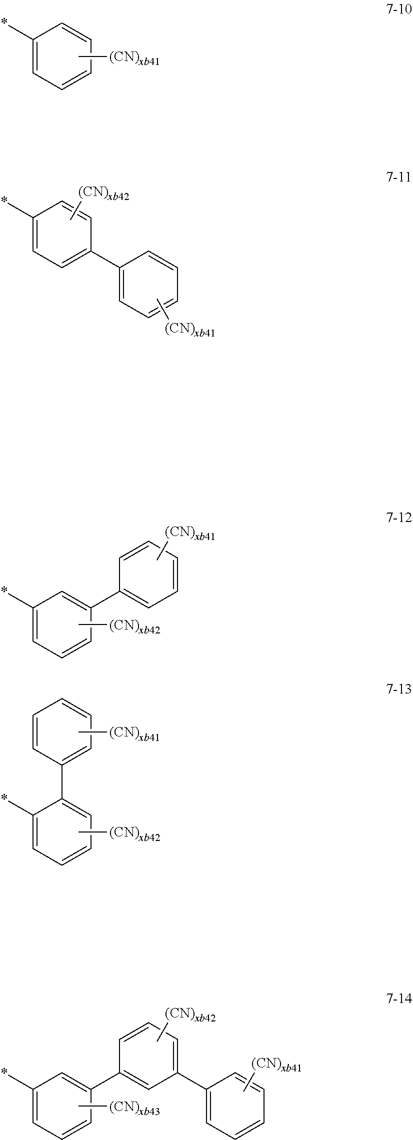

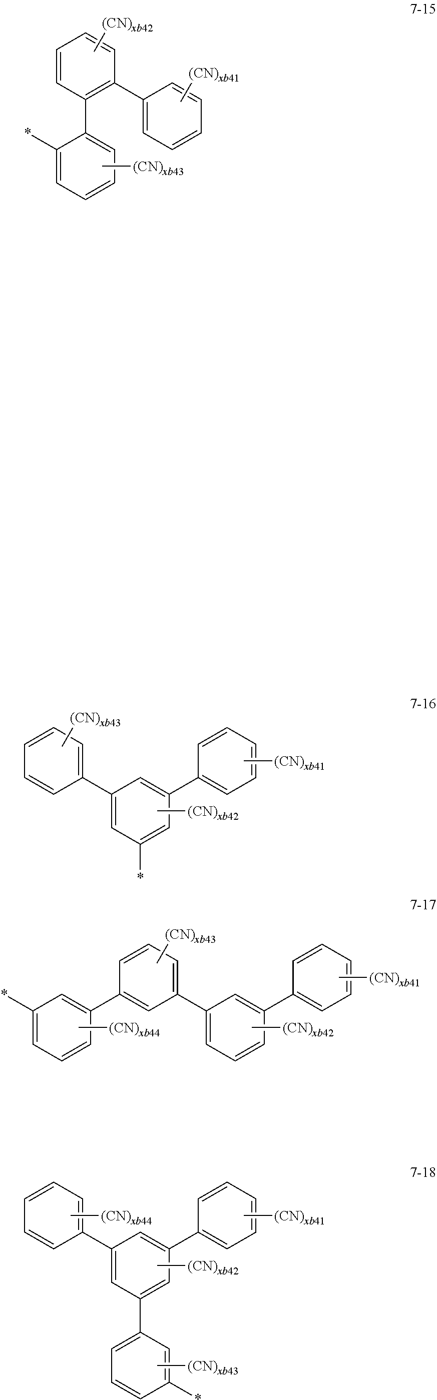

wherein, in Formulae 7-1 to 7-18,

xb4a to xb44 may each be 0, 1, or 2, provided that xb4 in Formula 7-10 may not be 0, xb4i+xb42 in Formulae 7-11 to 7-13 may not be 0, xb4i+xb42+xb43 in Formulae 7-14 to 7-16 may not be 0, xb4i+xb42+xb43+xb44 in Formulae 7-17 and 7-18 may not be 0, and * indicates a binding site to an adjacent atom.

In Formula F-1, at least two Ar₃₀₁(s) may be identical to or different from each other, and at least two L₃₀₁(s) may be identical to or different from each other. In Formula H-1, at least two L₄₀₁(s) may be identical to or different from each other, and at least two Ar₄₀₂(s) may be identical to or different from each other.

In an embodiment, the electron transporting host may include i) at least one a cyano group, a pyrimidine group, a pyrazine group, a triazine group, or any combination thereof, or ii) a triphenylene group, and the hole transporting host may include a carbazole group.

In one or more embodiments, the electron transporting host may include at least one cyano group.

The electron transporting host may be, for example, one compound of Groups HE1 to HE7, but embodiments are not limited thereto:

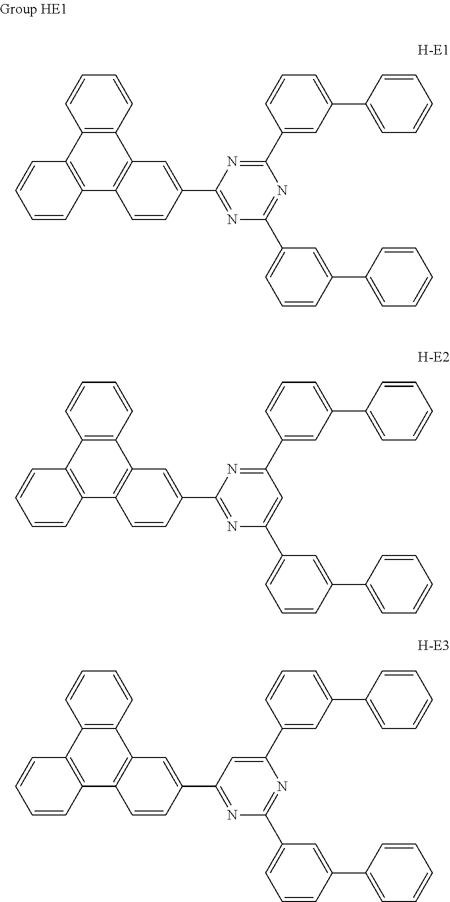 [SMILES: c1ccc(-c2cccc(-c3nc(-c4cccc(-c5ccccc5)c4)nc(-c4ccc5c6ccccc6c6ccccc6c5c4)n3)c2)cc1 ; c1ccc(-c2cccc(-c3cc(-c4cccc(-c5ccccc5)c4)nc(-c4ccc5c6ccccc6c6ccccc6c5c4)n3)c2)cc1 ; c1ccc(-c2cccc(-c3cc(-c4ccc5c6ccccc6c6ccccc6c5c4)nc(-c4cccc(-c5ccccc5)c4)n3)c2)cc1]

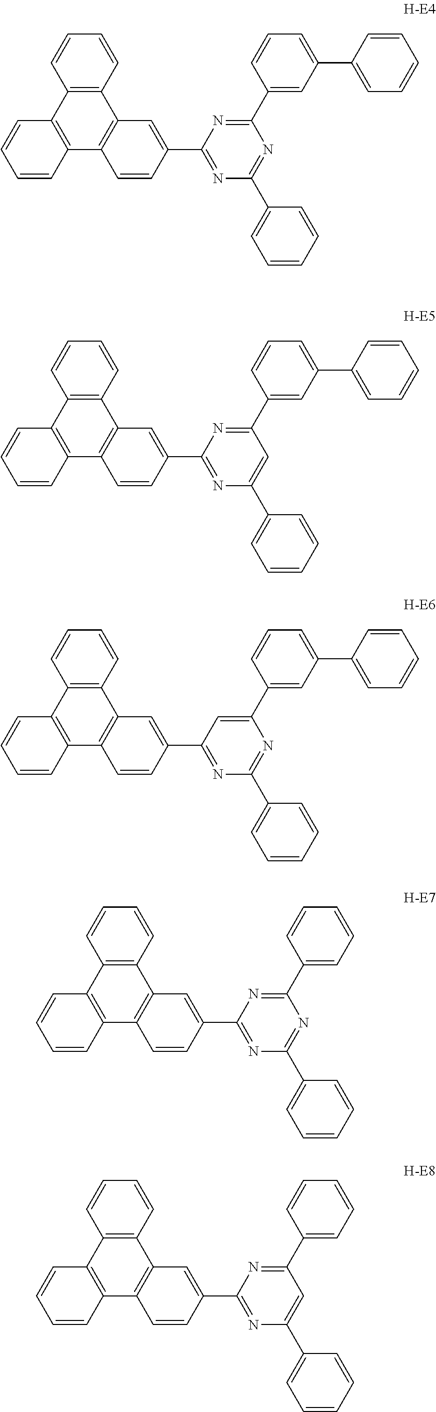

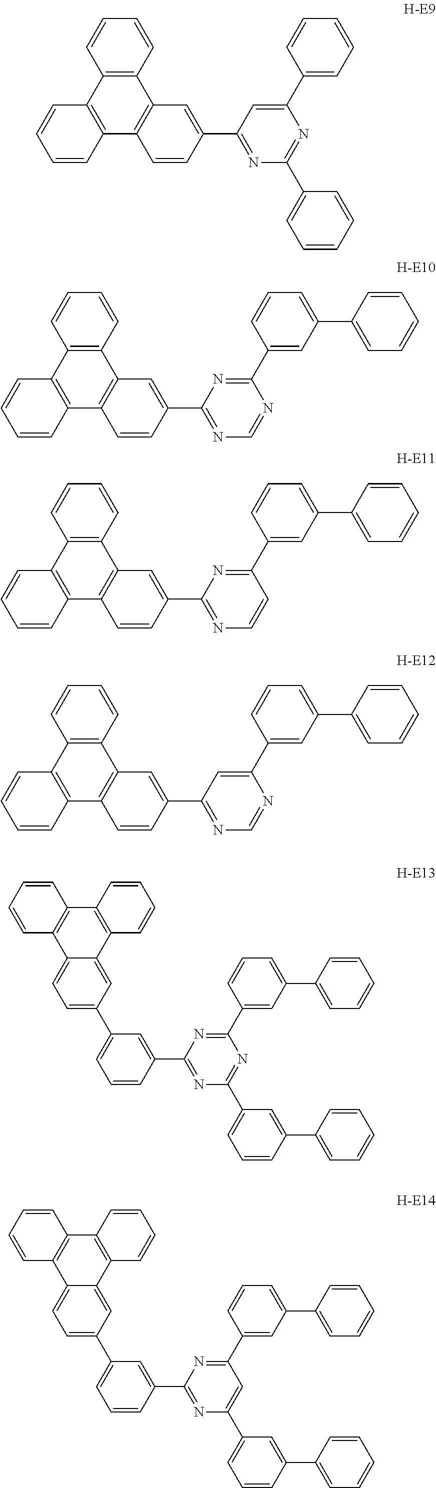

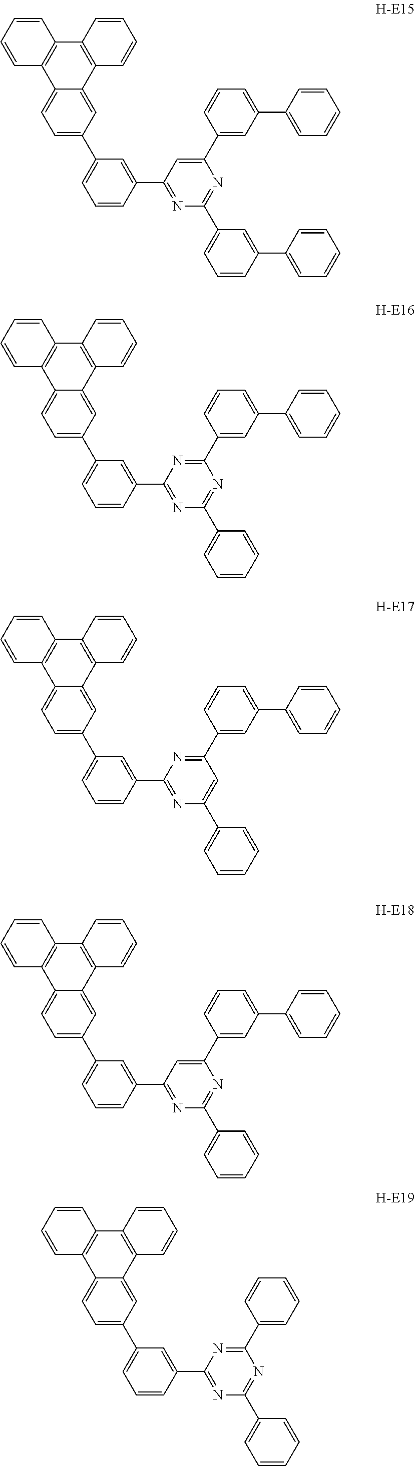 [SMILES: c1ccc(-c2cccc(-c3cc(-c4cccc(-c5ccc6c7ccccc7c7ccccc7c6c5)c4)nc(-c4cccc(-c5ccccc5)c4)n3)c2)cc1 ; c1ccc(-c2cccc(-c3nc(-c4ccccc4)nc(-c4cccc(-c5ccc6c7ccccc7c7ccccc7c6c5)c4)n3)c2)cc1 ; c1ccc(-c2cccc(-c3cc(-c4ccccc4)nc(-c4cccc(-c5ccc6c7ccccc7c7ccccc7c6c5)c4)n3)c2)cc1 ; c1ccc(-c2cccc(-c3cc(-c4cccc(-c5ccc6c7ccccc7c7ccccc7c6c5)c4)nc(-c4ccccc4)n3)c2)cc1 ; c1ccc(-c2nc(-c3ccccc3)nc(-c3cccc(-c4ccc5c6ccccc6c6ccccc6c5c4)c3)n2)cc1]

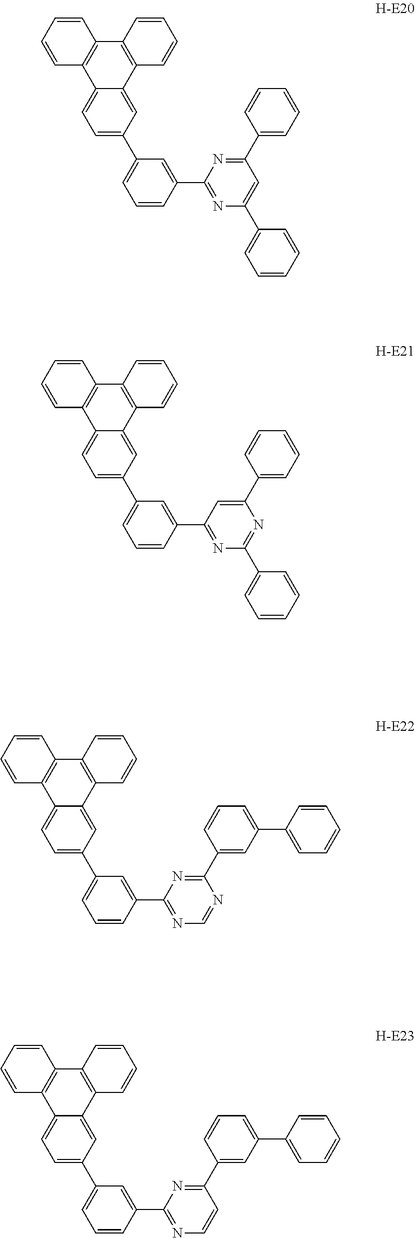 [SMILES: c1ccc(-c2cc(-c3ccccc3)nc(-c3cccc(-c4ccc5c6ccccc6c6ccccc6c5c4)c3)n2)cc1 ; c1ccc(-c2cc(-c3cccc(-c4ccc5c6ccccc6c6ccccc6c5c4)c3)nc(-c3ccccc3)n2)cc1 ; c1ccc(-c2cccc(-c3ncnc(-c4cccc(-c5ccc6c7ccccc7c7ccccc7c6c5)c4)n3)c2)cc1 ; c1ccc(-c2cccc(-c3ccnc(-c4cccc(-c5ccc6c7ccccc7c7ccccc7c6c5)c4)n3)c2)cc1]

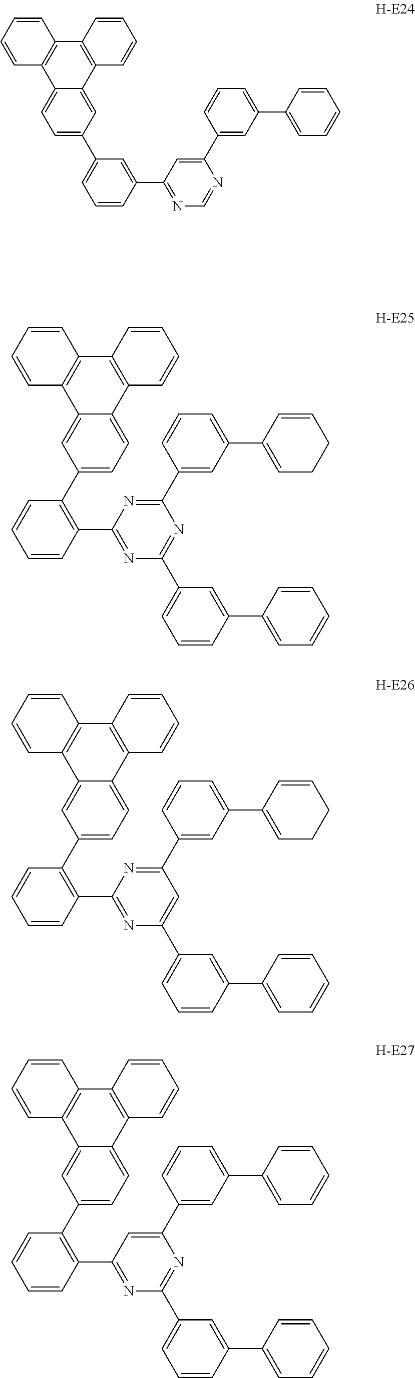 [SMILES: c1ccc(-c2cccc(-c3cc(-c4cccc(-c5ccc6c7ccccc7c7ccccc7c6c5)c4)ncn3)c2)cc1 ; C1=CC(c2cccc(-c3nc(-c4cccc(-c5ccccc5)c4)nc(-c4ccccc4-c4ccc5c6ccccc6c6ccccc6c5c4)n3)c2)=CCC1 ; C1=CC(c2cccc(-c3cc(-c4cccc(-c5ccccc5)c4)nc(-c4ccccc4-c4ccc5c6ccccc6c6ccccc6c5c4)n3)c2)=CCC1 ; c1ccc(-c2cccc(-c3cc(-c4ccccc4-c4ccc5c6ccccc6c6ccccc6c5c4)nc(-c4cccc(-c5ccccc5)c4)n3)c2)cc1]

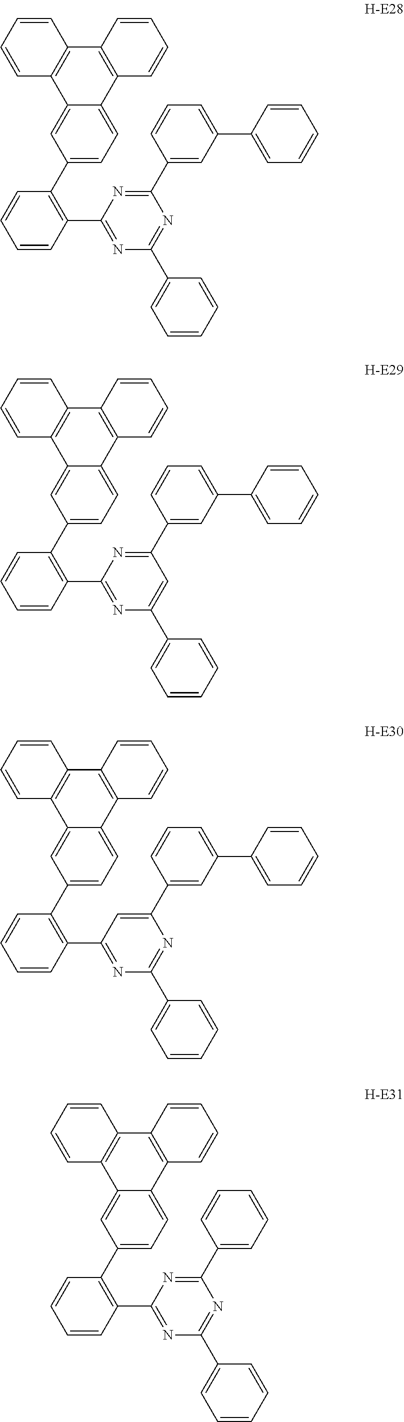

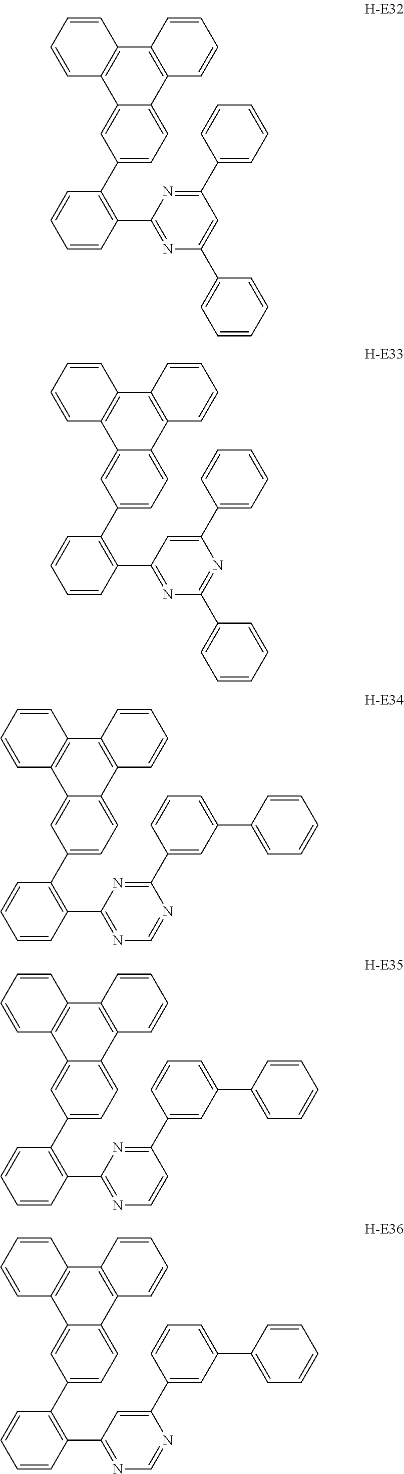

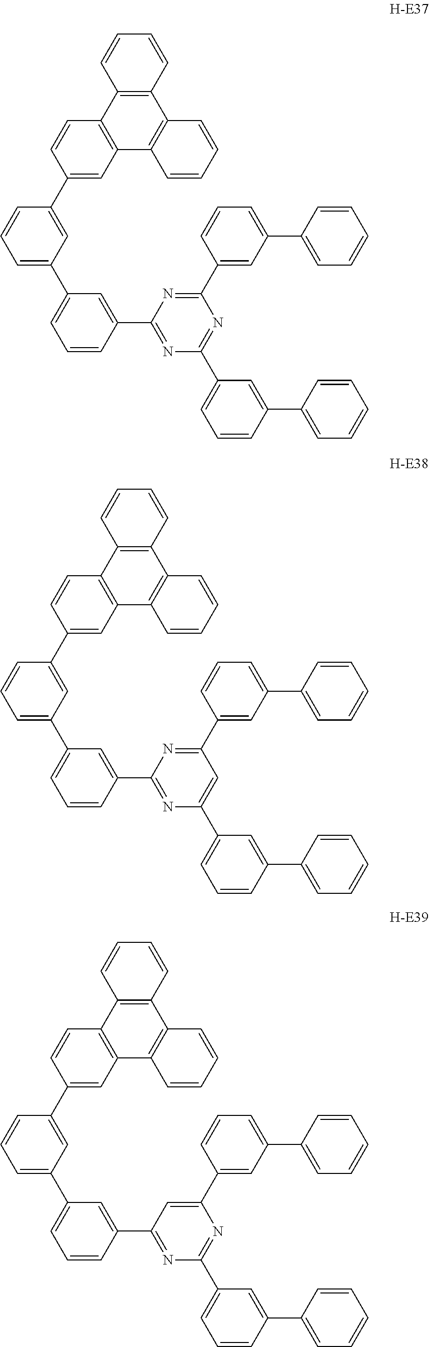 [SMILES: c1ccc(-c2cccc(-c3nc(-c4cccc(-c5ccccc5)c4)nc(-c4cccc(-c5cccc(-c6ccc7c8ccccc8c8ccccc8c7c6)c5)c4)n3)c2)cc1 ; c1ccc(-c2cccc(-c3cc(-c4cccc(-c5ccccc5)c4)nc(-c4cccc(-c5cccc(-c6ccc7c8ccccc8c8ccccc8c7c6)c5)c4)n3)c2)cc1 ; c1ccc(-c2cccc(-c3cc(-c4cccc(-c5cccc(-c6ccc7c8ccccc8c8ccccc8c7c6)c5)c4)nc(-c4cccc(-c5ccccc5)c4)n3)c2)cc1]

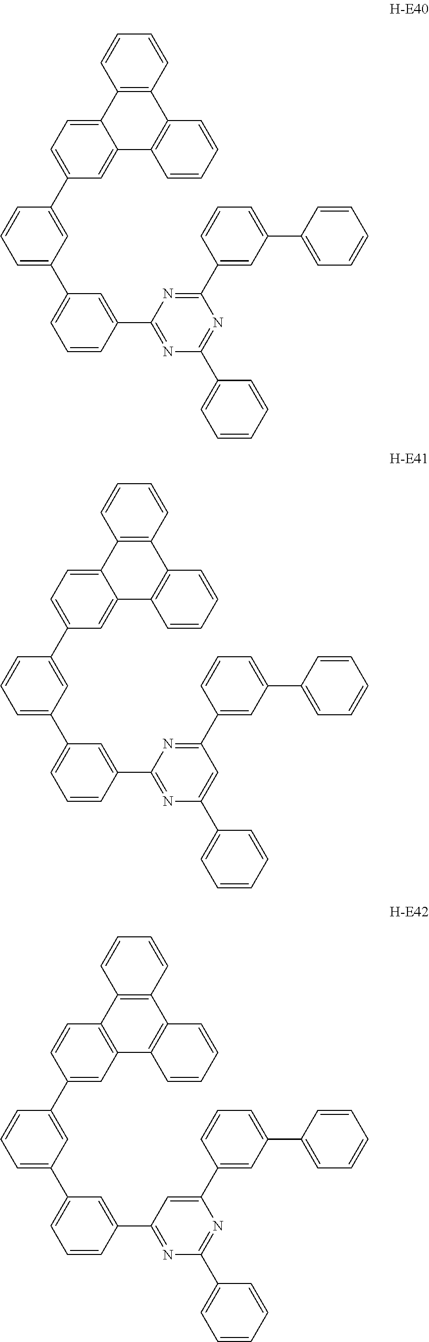 [SMILES: c1ccc(-c2cccc(-c3nc(-c4ccccc4)nc(-c4cccc(-c5cccc(-c6ccc7c8ccccc8c8ccccc8c7c6)c5)c4)n3)c2)cc1 ; c1ccc(-c2cccc(-c3cc(-c4ccccc4)nc(-c4cccc(-c5cccc(-c6ccc7c8ccccc8c8ccccc8c7c6)c5)c4)n3)c2)cc1 ; c1ccc(-c2cccc(-c3cc(-c4cccc(-c5cccc(-c6ccc7c8ccccc8c8ccccc8c7c6)c5)c4)nc(-c4ccccc4)n3)c2)cc1]

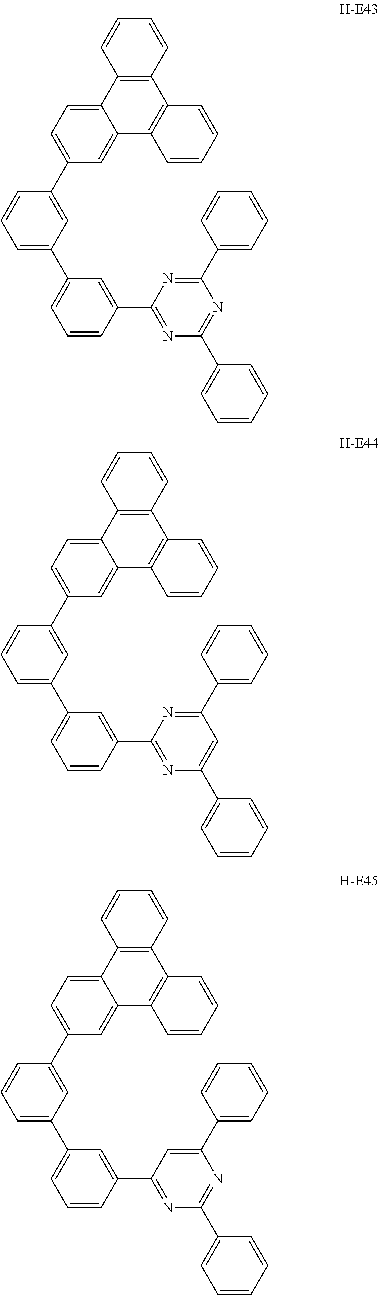 [SMILES: c1ccc(-c2nc(-c3ccccc3)nc(-c3cccc(-c4cccc(-c5ccc6c7ccccc7c7ccccc7c6c5)c4)c3)n2)cc1 ; c1ccc(-c2cc(-c3ccccc3)nc(-c3cccc(-c4cccc(-c5ccc6c7ccccc7c7ccccc7c6c5)c4)c3)n2)cc1 ; c1ccc(-c2cc(-c3cccc(-c4cccc(-c5ccc6c7ccccc7c7ccccc7c6c5)c4)c3)nc(-c3ccccc3)n2)cc1]

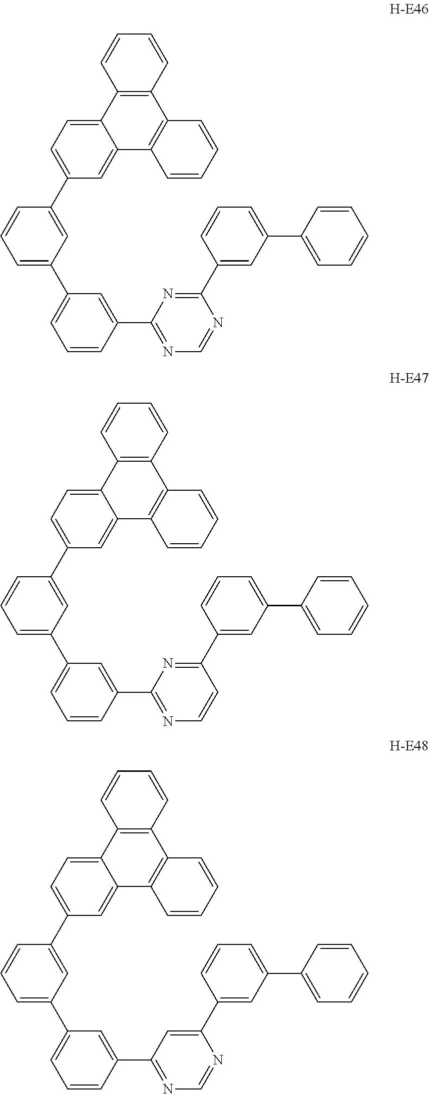 [SMILES: c1ccc(-c2cccc(-c3ncnc(-c4cccc(-c5cccc(-c6ccc7c8ccccc8c8ccccc8c7c6)c5)c4)n3)c2)cc1 ; c1ccc(-c2cccc(-c3ccnc(-c4cccc(-c5cccc(-c6ccc7c8ccccc8c8ccccc8c7c6)c5)c4)n3)c2)cc1 ; c1ccc(-c2cccc(-c3cc(-c4cccc(-c5cccc(-c6ccc7c8ccccc8c8ccccc8c7c6)c5)c4)ncn3)c2)cc1]

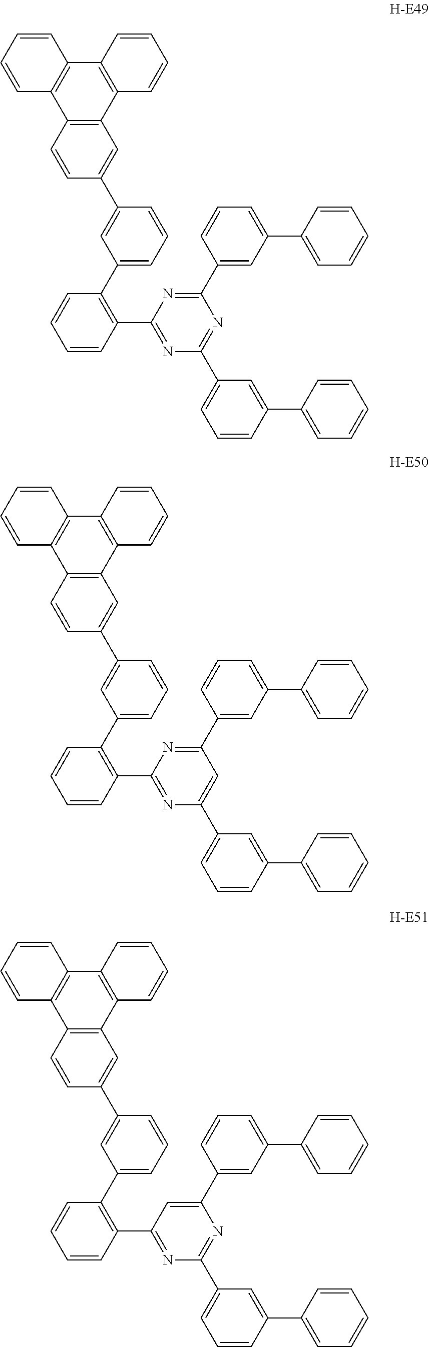 [SMILES: c1ccc(-c2cccc(-c3nc(-c4cccc(-c5ccccc5)c4)nc(-c4ccccc4-c4cccc(-c5ccc6c7ccccc7c7ccccc7c6c5)c4)n3)c2)cc1 ; c1ccc(-c2cccc(-c3cc(-c4cccc(-c5ccccc5)c4)nc(-c4ccccc4-c4cccc(-c5ccc6c7ccccc7c7ccccc7c6c5)c4)n3)c2)cc1 ; c1ccc(-c2cccc(-c3cc(-c4ccccc4-c4cccc(-c5ccc6c7ccccc7c7ccccc7c6c5)c4)nc(-c4cccc(-c5ccccc5)c4)n3)c2)cc1]

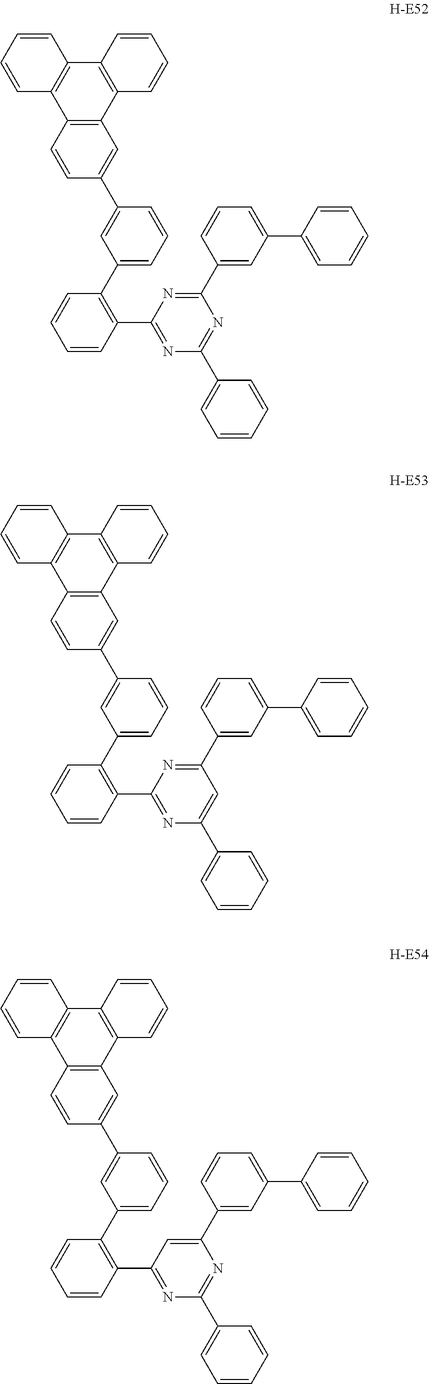 [SMILES: c1ccc(-c2cccc(-c3nc(-c4ccccc4)nc(-c4ccccc4-c4cccc(-c5ccc6c7ccccc7c7ccccc7c6c5)c4)n3)c2)cc1 ; c1ccc(-c2cccc(-c3cc(-c4ccccc4)nc(-c4ccccc4-c4cccc(-c5ccc6c7ccccc7c7ccccc7c6c5)c4)n3)c2)cc1 ; c1ccc(-c2cccc(-c3cc(-c4ccccc4-c4cccc(-c5ccc6c7ccccc7c7ccccc7c6c5)c4)nc(-c4ccccc4)n3)c2)cc1]

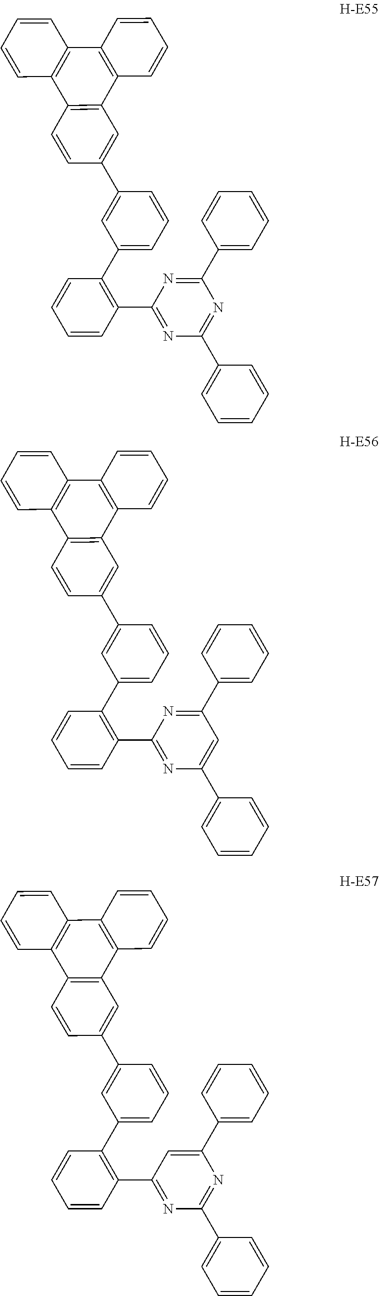 [SMILES: c1ccc(-c2nc(-c3ccccc3)nc(-c3ccccc3-c3cccc(-c4ccc5c6ccccc6c6ccccc6c5c4)c3)n2)cc1 ; c1ccc(-c2cc(-c3ccccc3)nc(-c3ccccc3-c3cccc(-c4ccc5c6ccccc6c6ccccc6c5c4)c3)n2)cc1 ; c1ccc(-c2cc(-c3ccccc3-c3cccc(-c4ccc5c6ccccc6c6ccccc6c5c4)c3)nc(-c3ccccc3)n2)cc1]

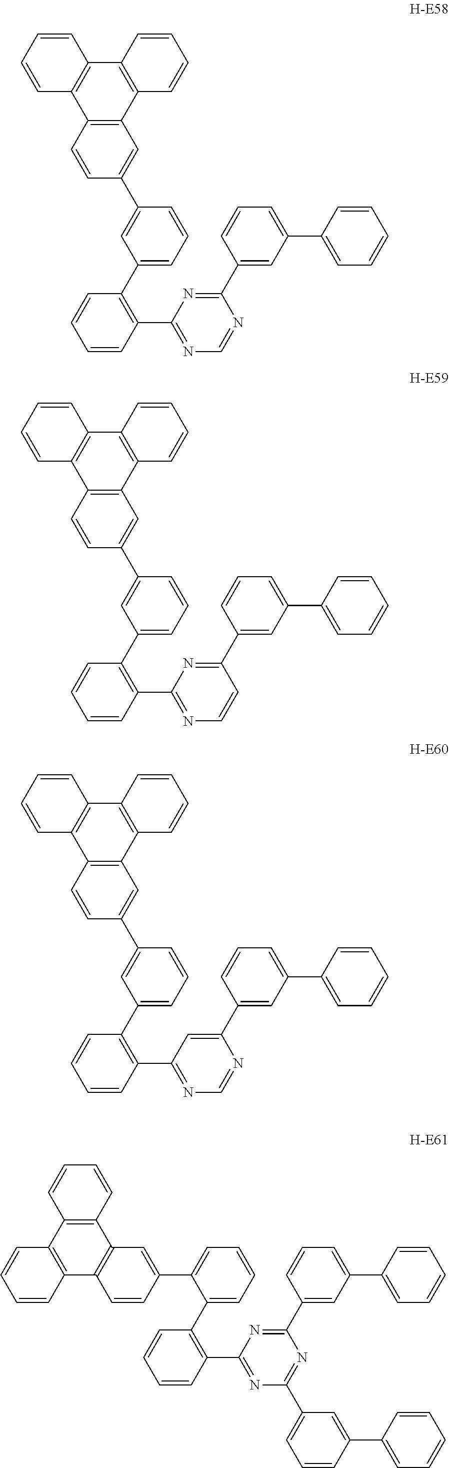 [SMILES: c1ccc(-c2cccc(-c3ncnc(-c4ccccc4-c4cccc(-c5ccc6c7ccccc7c7ccccc7c6c5)c4)n3)c2)cc1 ; c1ccc(-c2cccc(-c3ccnc(-c4ccccc4-c4cccc(-c5ccc6c7ccccc7c7ccccc7c6c5)c4)n3)c2)cc1 ; c1ccc(-c2cccc(-c3cc(-c4ccccc4-c4cccc(-c5ccc6c7ccccc7c7ccccc7c6c5)c4)ncn3)c2)cc1 ; c1ccc(-c2cccc(-c3nc(-c4cccc(-c5ccccc5)c4)nc(-c4ccccc4-c4ccccc4-c4ccc5c6ccccc6c6ccccc6c5c4)n3)c2)cc1]

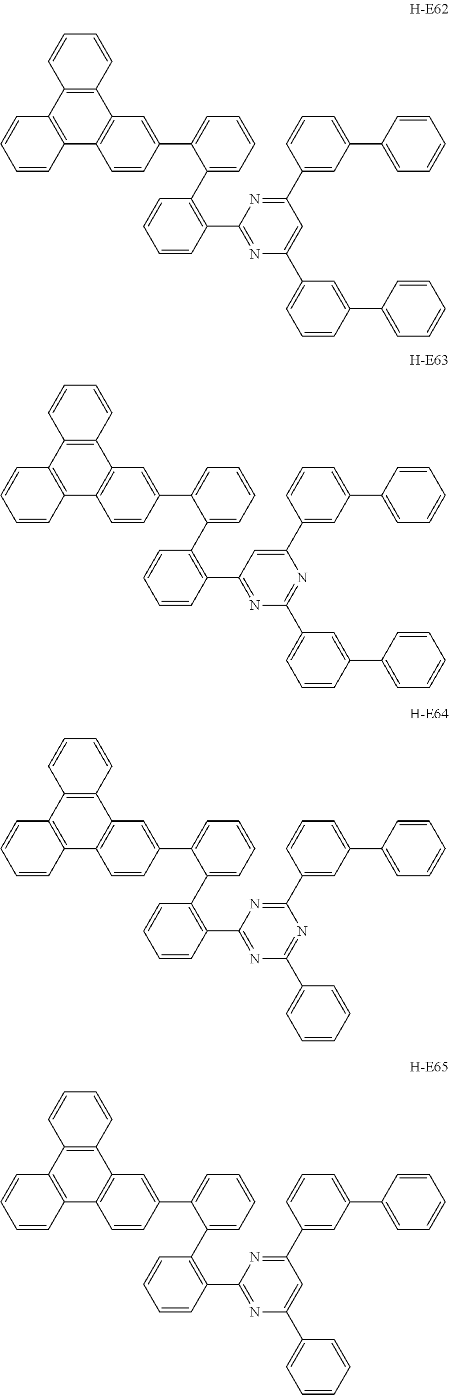 [SMILES: c1ccc(-c2cccc(-c3cc(-c4cccc(-c5ccccc5)c4)nc(-c4ccccc4-c4ccccc4-c4ccc5c6ccccc6c6ccccc6c5c4)n3)c2)cc1 ; c1ccc(-c2cccc(-c3cc(-c4ccccc4-c4ccccc4-c4ccc5c6ccccc6c6ccccc6c5c4)nc(-c4cccc(-c5ccccc5)c4)n3)c2)cc1 ; c1ccc(-c2cccc(-c3nc(-c4ccccc4)nc(-c4ccccc4-c4ccccc4-c4ccc5c6ccccc6c6ccccc6c5c4)n3)c2)cc1 ; c1ccc(-c2cccc(-c3cc(-c4ccccc4)nc(-c4ccccc4-c4ccccc4-c4ccc5c6ccccc6c6ccccc6c5c4)n3)c2)cc1]

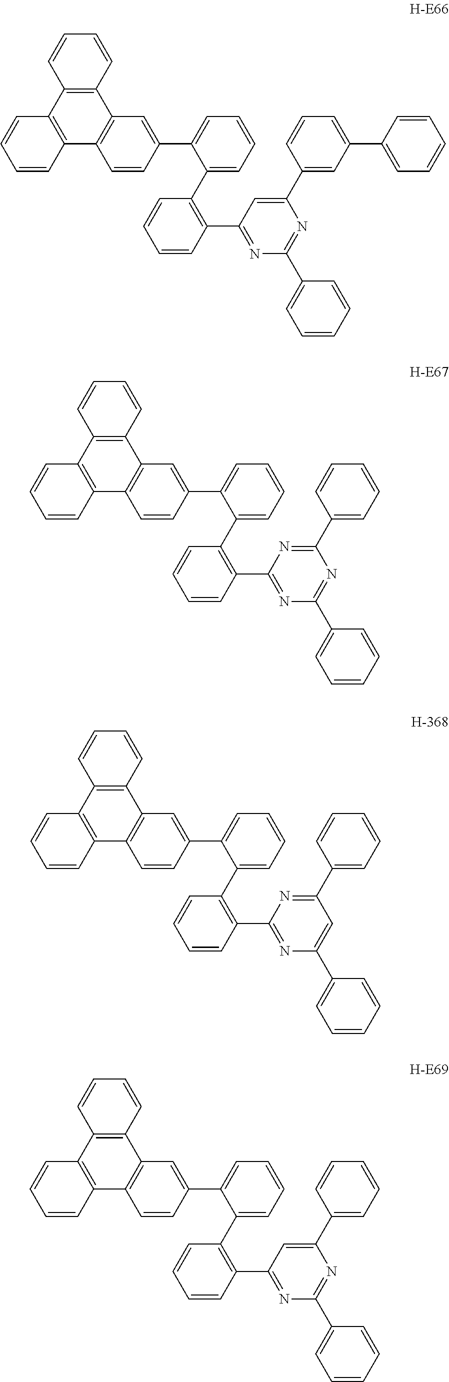 [SMILES: c1ccc(-c2cccc(-c3cc(-c4ccccc4-c4ccccc4-c4ccc5c6ccccc6c6ccccc6c5c4)nc(-c4ccccc4)n3)c2)cc1 ; c1ccc(-c2nc(-c3ccccc3)nc(-c3ccccc3-c3ccccc3-c3ccc4c5ccccc5c5ccccc5c4c3)n2)cc1 ; c1ccc(-c2cc(-c3ccccc3)nc(-c3ccccc3-c3ccccc3-c3ccc4c5ccccc5c5ccccc5c4c3)n2)cc1 ; c1ccc(-c2cc(-c3ccccc3-c3ccccc3-c3ccc4c5ccccc5c5ccccc5c4c3)nc(-c3ccccc3)n2)cc1]

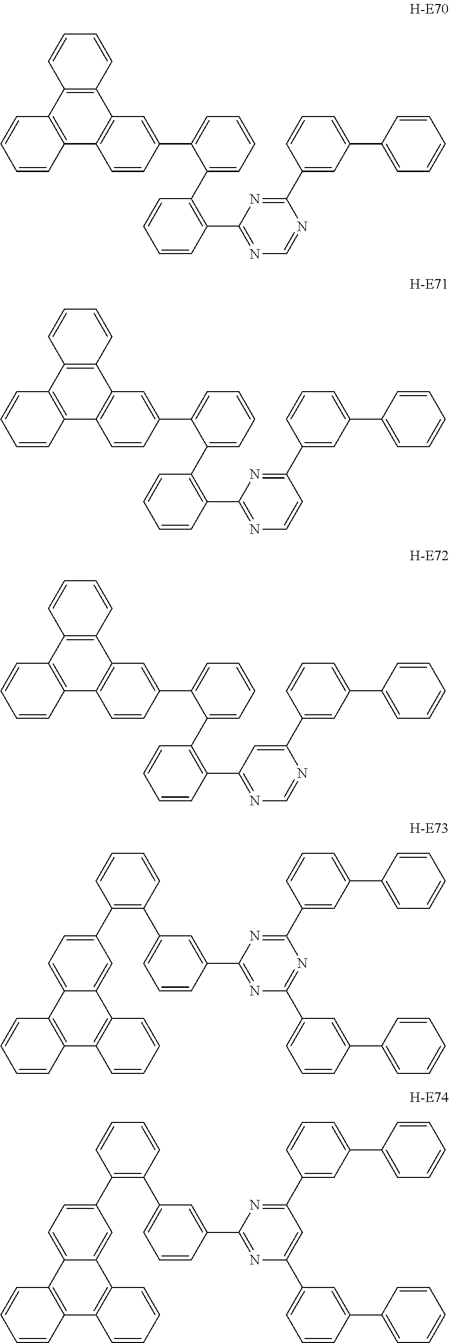 [SMILES: c1ccc(-c2cccc(-c3ncnc(-c4ccccc4-c4ccccc4-c4ccc5c6ccccc6c6ccccc6c5c4)n3)c2)cc1 ; c1ccc(-c2cccc(-c3ccnc(-c4ccccc4-c4ccccc4-c4ccc5c6ccccc6c6ccccc6c5c4)n3)c2)cc1 ; c1ccc(-c2cccc(-c3cc(-c4ccccc4-c4ccccc4-c4ccc5c6ccccc6c6ccccc6c5c4)ncn3)c2)cc1 ; c1ccc(-c2cccc(-c3nc(-c4cccc(-c5ccccc5)c4)nc(-c4cccc(-c5ccccc5-c5ccc6c7ccccc7c7ccccc7c6c5)c4)n3)c2)cc1 ; c1ccc(-c2cccc(-c3cc(-c4cccc(-c5ccccc5)c4)nc(-c4cccc(-c5ccccc5-c5ccc6c7ccccc7c7ccccc7c6c5)c4)n3)c2)cc1]

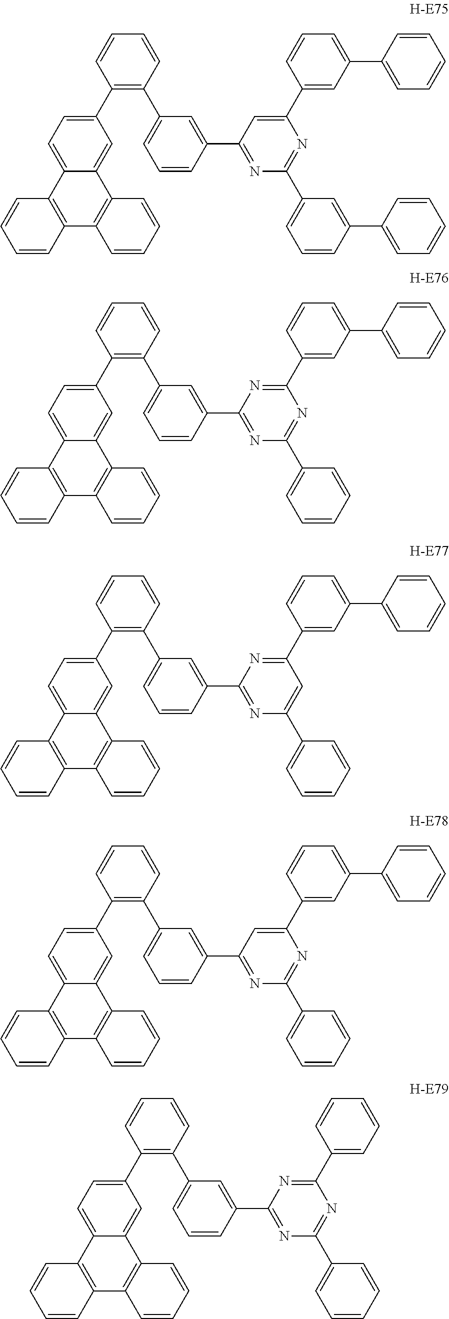

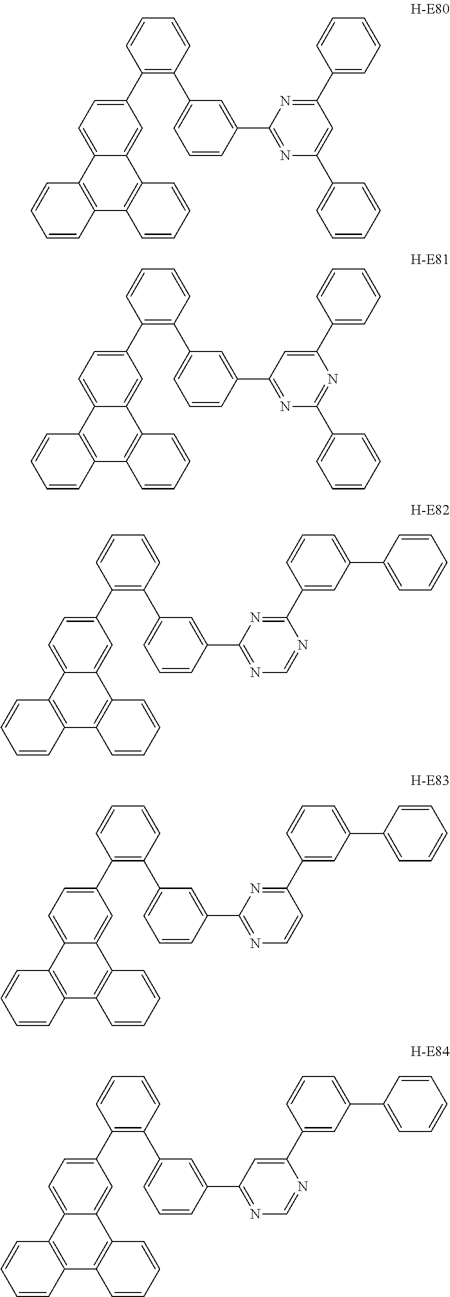

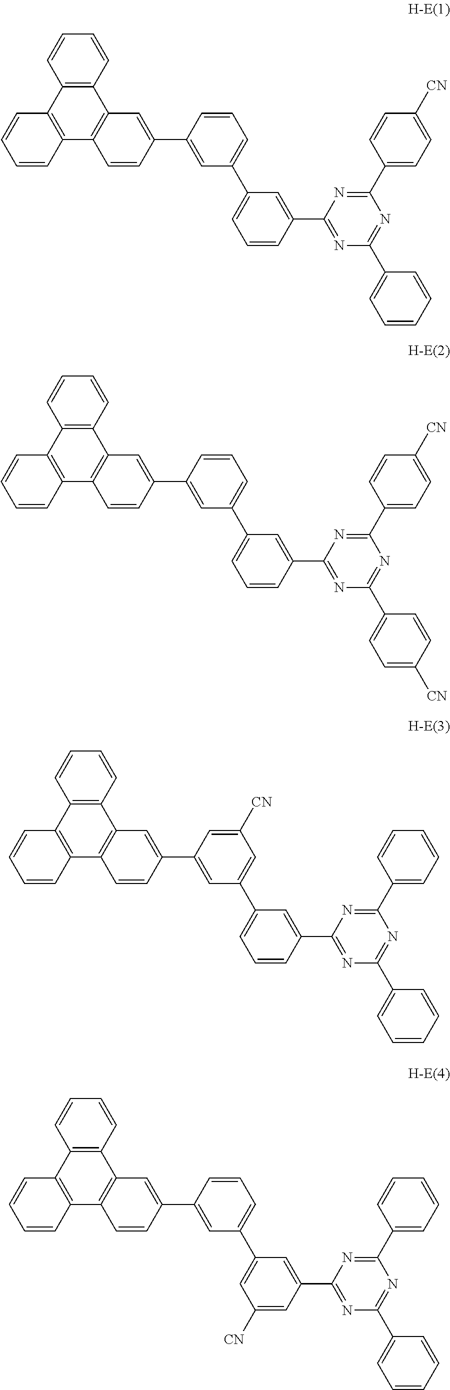

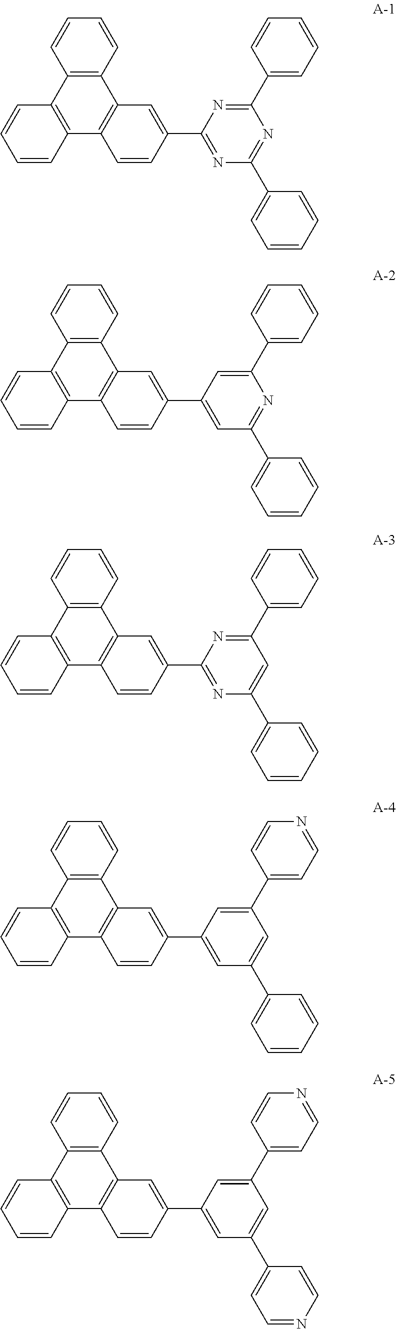

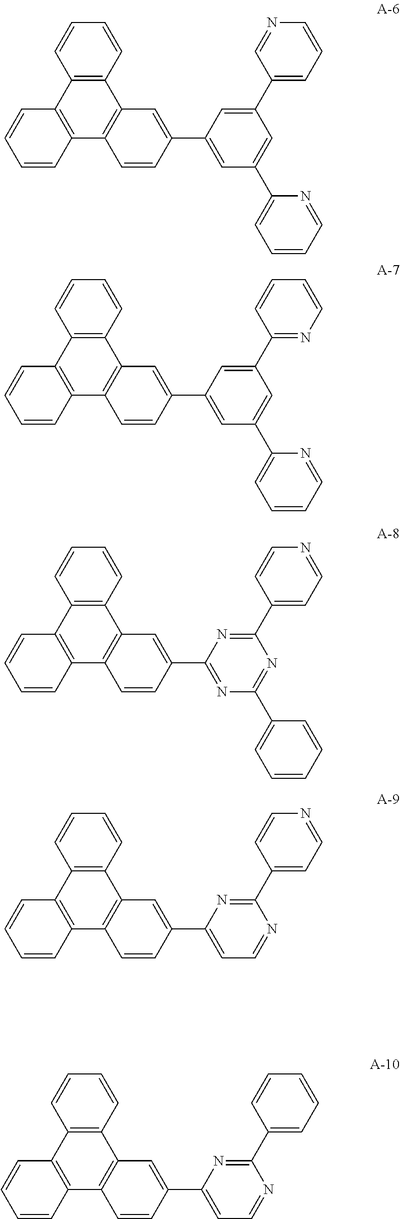

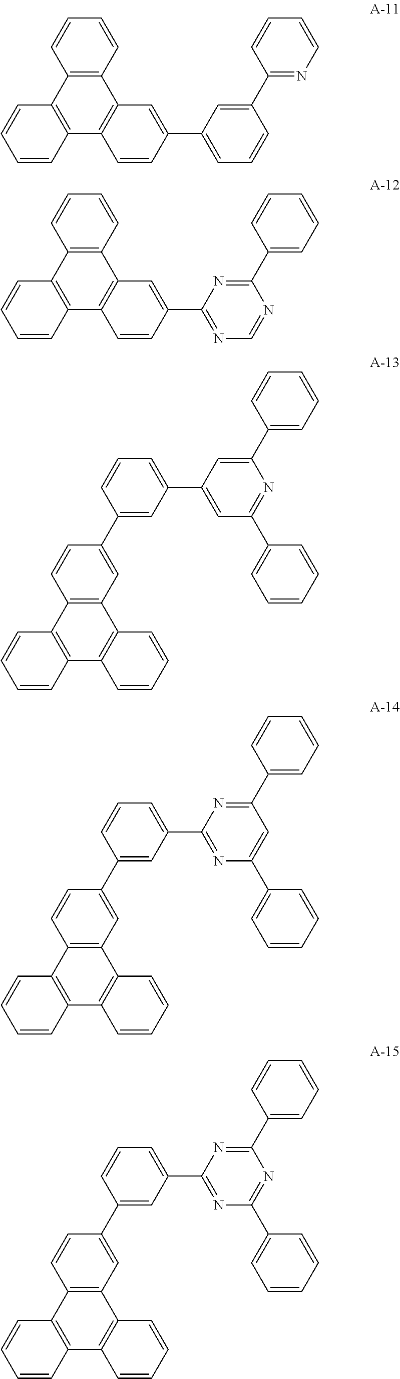

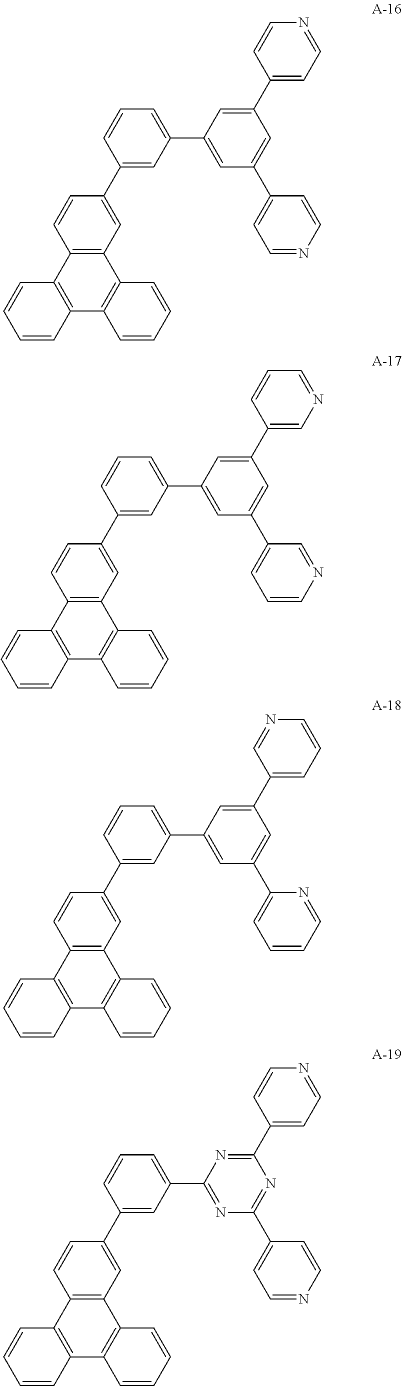 [SMILES: c1cc(-c2cc(-c3ccncc3)cc(-c3ccncc3)c2)cc(-c2ccc3c4ccccc4c4ccccc4c3c2)c1 ; c1cncc(-c2cc(-c3cccnc3)cc(-c3cccc(-c4ccc5c6ccccc6c6ccccc6c5c4)c3)c2)c1 ; c1ccc(-c2cc(-c3cccnc3)cc(-c3cccc(-c4ccc5c6ccccc6c6ccccc6c5c4)c3)c2)nc1 ; c1cc(-c2ccc3c4ccccc4c4ccccc4c3c2)cc(-c2nc(-c3ccncc3)nc(-c3ccncc3)n2)c1]

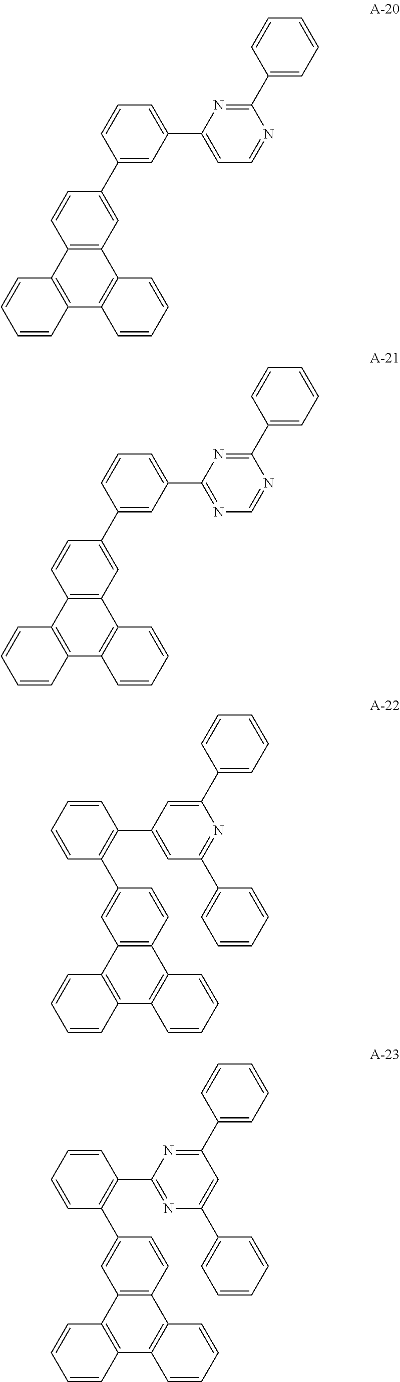

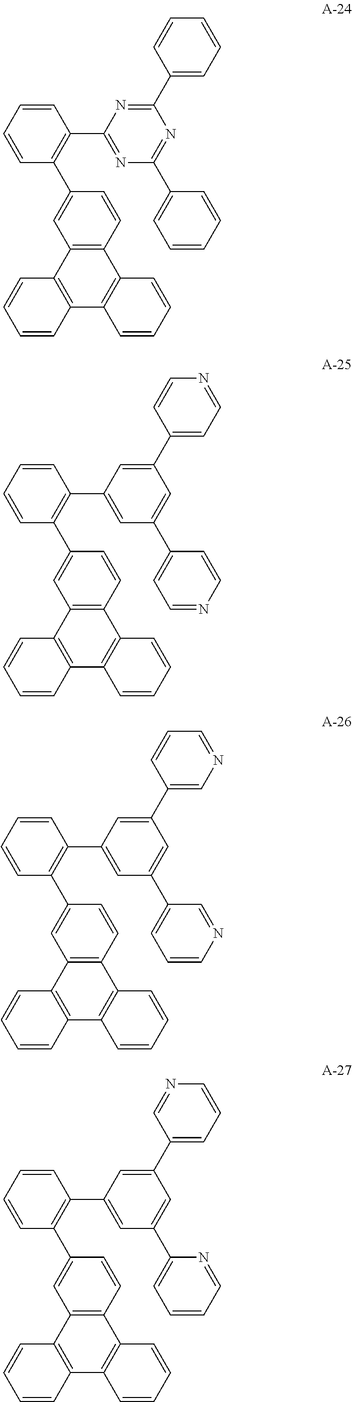

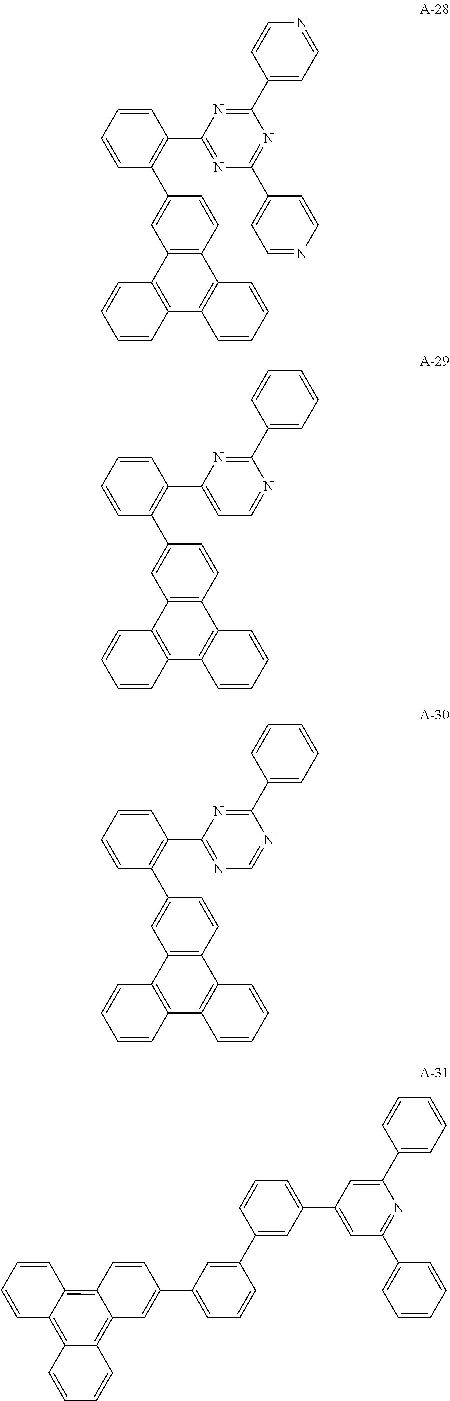

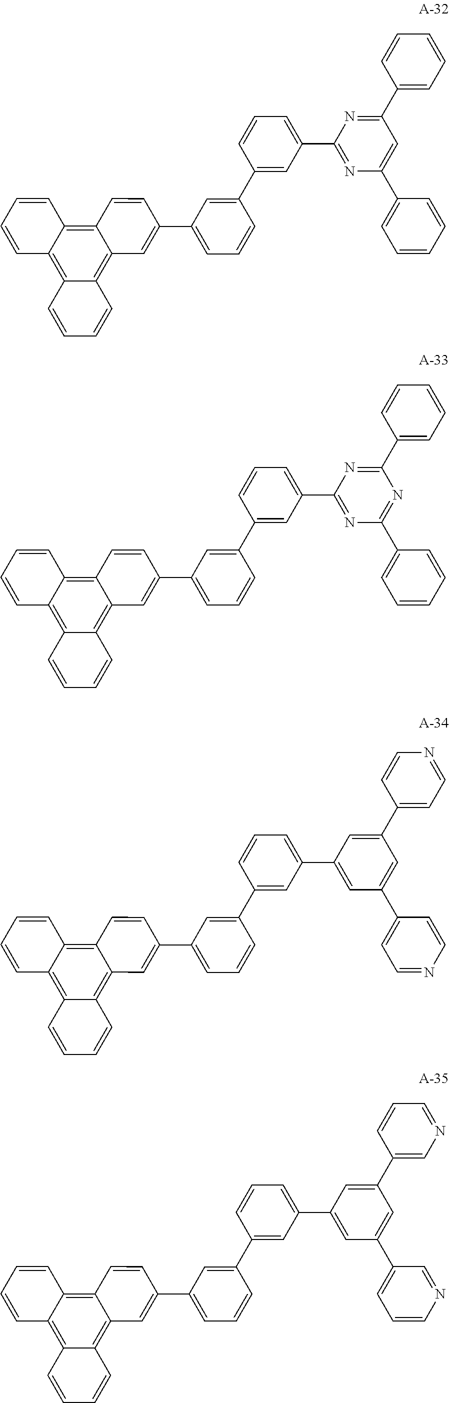 [SMILES: c1ccc(-c2cc(-c3ccccc3)nc(-c3cccc(-c4cccc(-c5ccc6c7ccccc7c7ccccc7c6c5)c4)c3)n2)cc1 ; c1ccc(-c2nc(-c3ccccc3)nc(-c3cccc(-c4cccc(-c5ccc6c7ccccc7c7ccccc7c6c5)c4)c3)n2)cc1 ; c1cc(-c2cccc(-c3ccc4c5ccccc5c5ccccc5c4c3)c2)cc(-c2cc(-c3ccncc3)cc(-c3ccncc3)c2)c1 ; c1cncc(-c2cc(-c3cccnc3)cc(-c3cccc(-c4cccc(-c5ccc6c7ccccc7c7ccccc7c6c5)c4)c3)c2)c1]

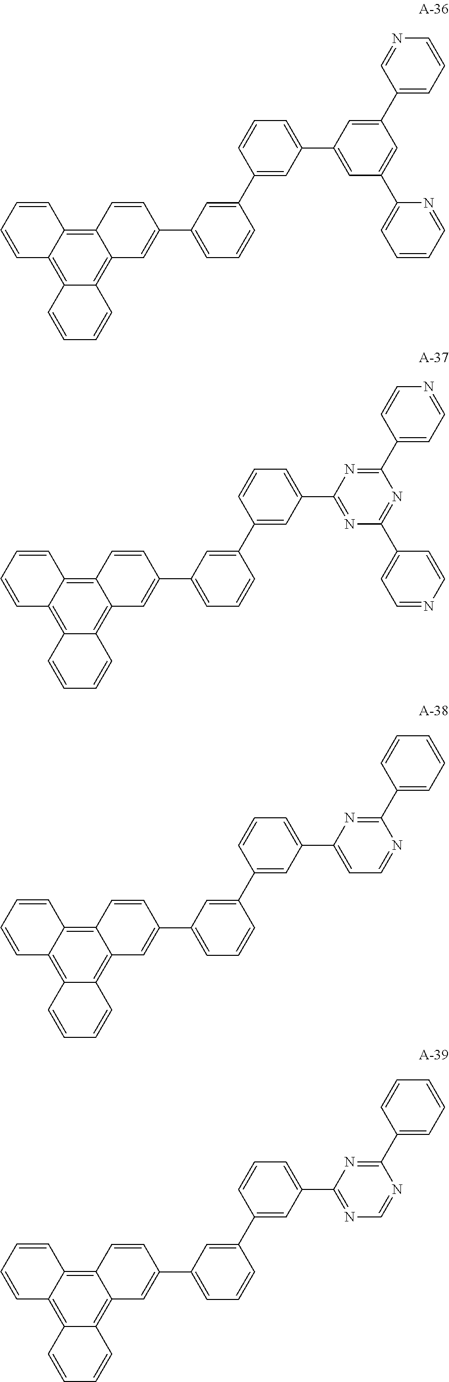 [SMILES: c1ccc(-c2cc(-c3cccnc3)cc(-c3cccc(-c4cccc(-c5ccc6c7ccccc7c7ccccc7c6c5)c4)c3)c2)nc1 ; c1cc(-c2cccc(-c3nc(-c4ccncc4)nc(-c4ccncc4)n3)c2)cc(-c2ccc3c4ccccc4c4ccccc4c3c2)c1 ; c1ccc(-c2nccc(-c3cccc(-c4cccc(-c5ccc6c7ccccc7c7ccccc7c6c5)c4)c3)n2)cc1 ; c1ccc(-c2ncnc(-c3cccc(-c4cccc(-c5ccc6c7ccccc7c7ccccc7c6c5)c4)c3)n2)cc1]

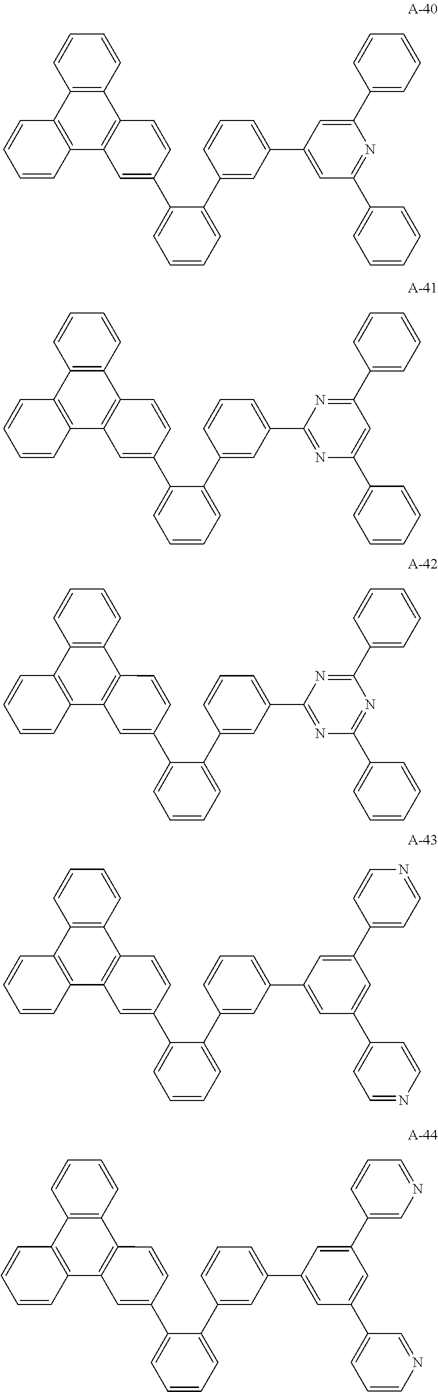

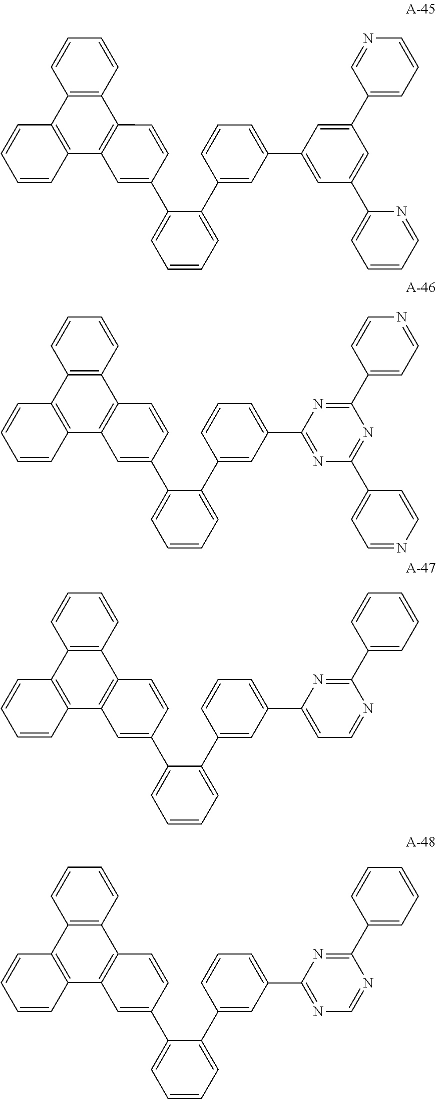

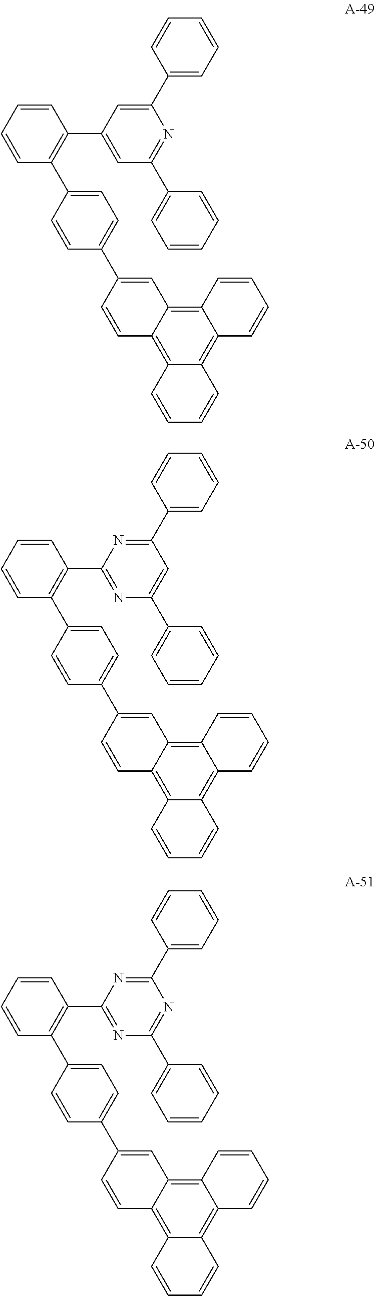

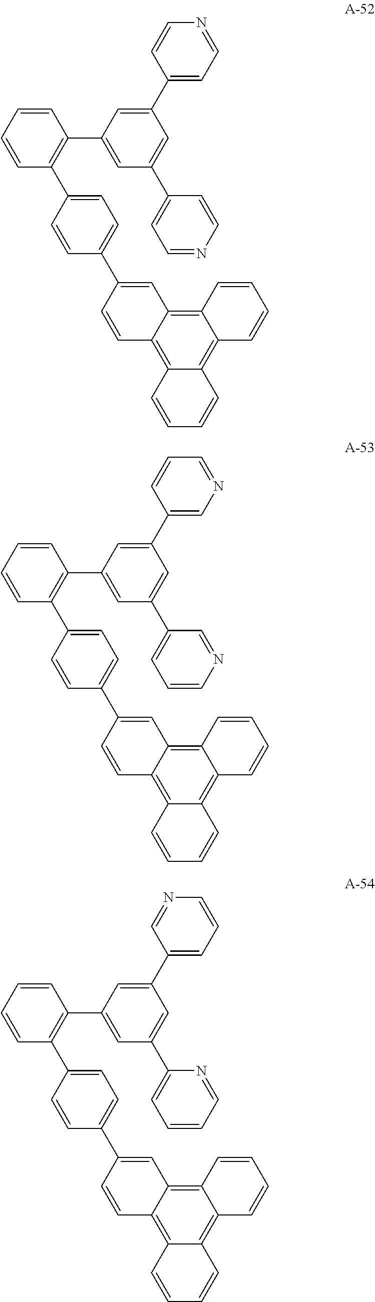

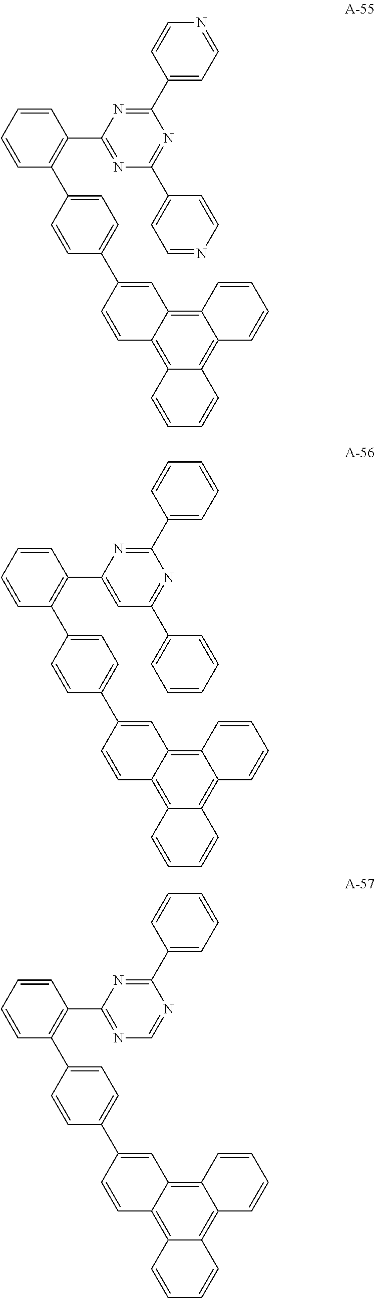

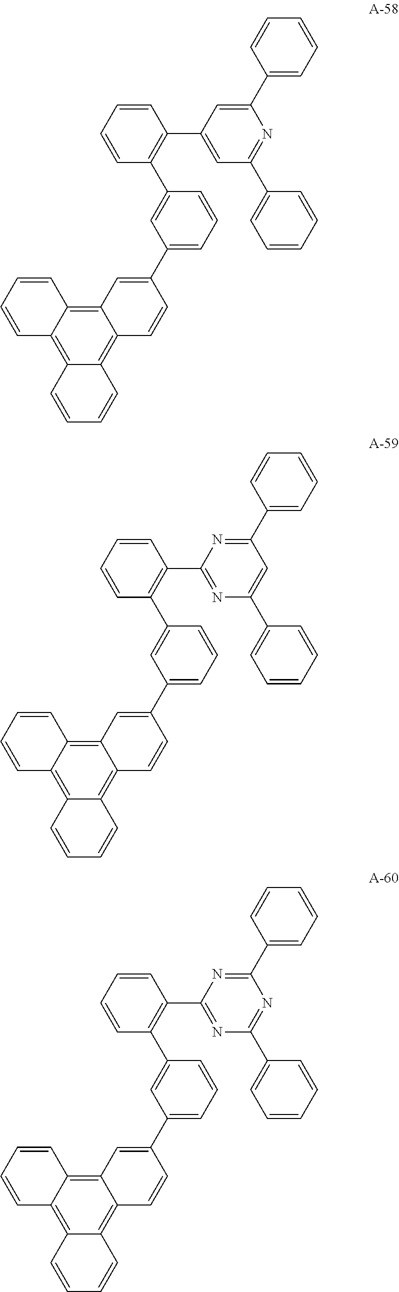 [SMILES: c1ccc(-c2cc(-c3ccccc3-c3cccc(-c4ccc5c6ccccc6c6ccccc6c5c4)c3)cc(-c3ccccc3)n2)cc1 ; c1ccc(-c2cc(-c3ccccc3)nc(-c3ccccc3-c3cccc(-c4ccc5c6ccccc6c6ccccc6c5c4)c3)n2)cc1 ; c1ccc(-c2nc(-c3ccccc3)nc(-c3ccccc3-c3cccc(-c4ccc5c6ccccc6c6ccccc6c5c4)c3)n2)cc1]

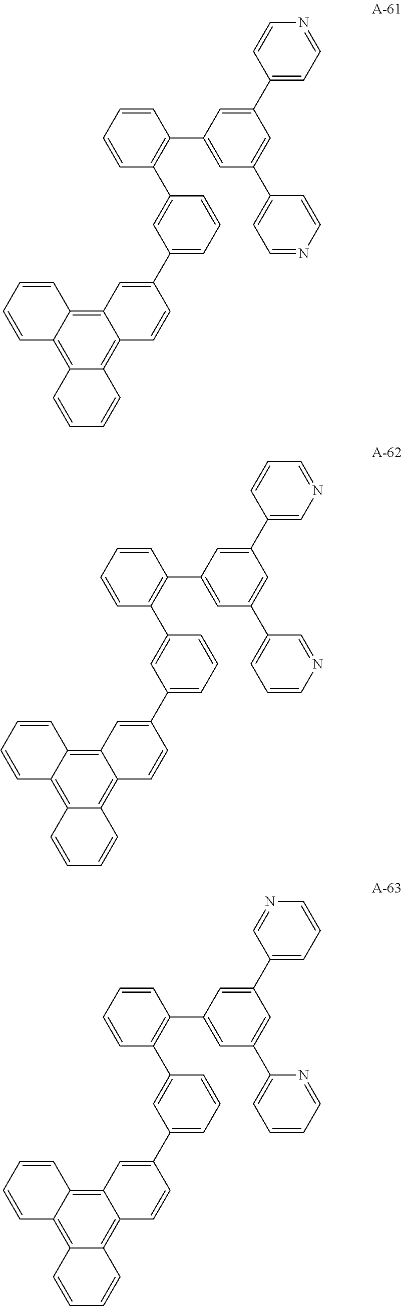 [SMILES: c1cc(-c2ccc3c4ccccc4c4ccccc4c3c2)cc(-c2ccccc2-c2cc(-c3ccncc3)cc(-c3ccncc3)c2)c1 ; c1cncc(-c2cc(-c3cccnc3)cc(-c3ccccc3-c3cccc(-c4ccc5c6ccccc6c6ccccc6c5c4)c3)c2)c1 ; c1ccc(-c2cc(-c3cccnc3)cc(-c3ccccc3-c3cccc(-c4ccc5c6ccccc6c6ccccc6c5c4)c3)c2)nc1]

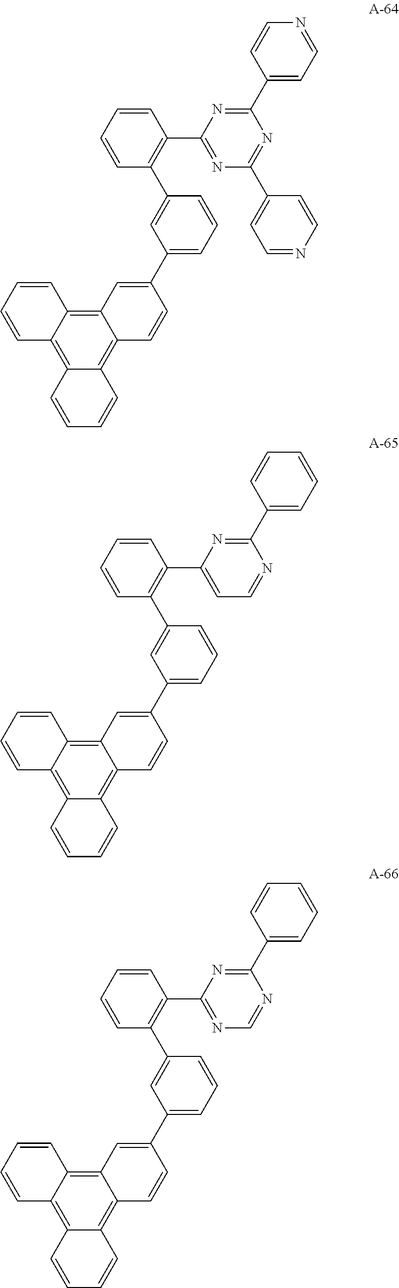 [SMILES: c1cc(-c2ccc3c4ccccc4c4ccccc4c3c2)cc(-c2ccccc2-c2nc(-c3ccncc3)nc(-c3ccncc3)n2)c1 ; c1ccc(-c2nccc(-c3ccccc3-c3cccc(-c4ccc5c6ccccc6c6ccccc6c5c4)c3)n2)cc1 ; c1ccc(-c2ncnc(-c3ccccc3-c3cccc(-c4ccc5c6ccccc6c6ccccc6c5c4)c3)n2)cc1]

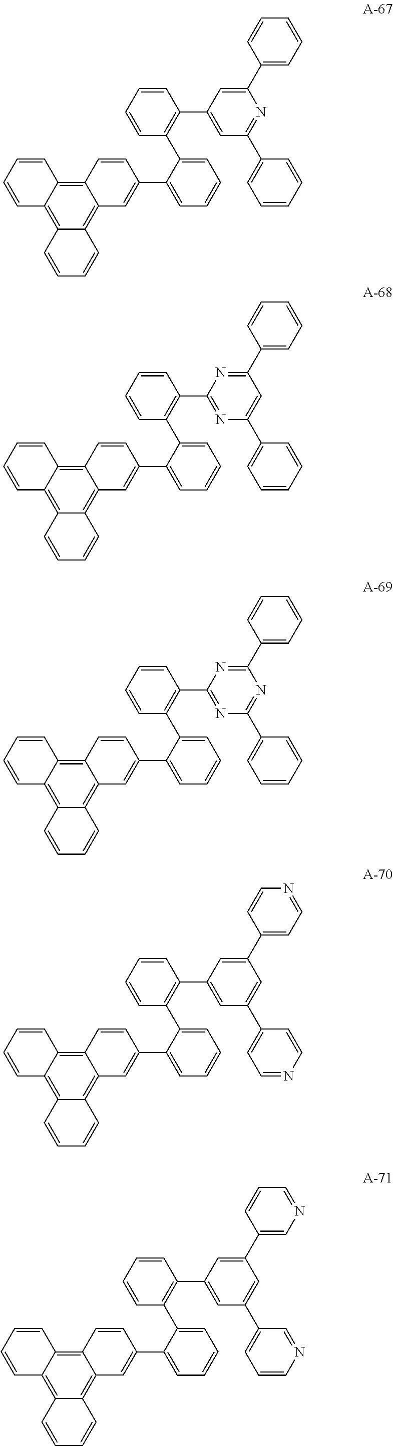 [SMILES: c1ccc(-c2cc(-c3ccccc3-c3ccccc3-c3ccc4c5ccccc5c5ccccc5c4c3)cc(-c3ccccc3)n2)cc1 ; c1ccc(-c2cc(-c3ccccc3)nc(-c3ccccc3-c3ccccc3-c3ccc4c5ccccc5c5ccccc5c4c3)n2)cc1 ; c1ccc(-c2nc(-c3ccccc3)nc(-c3ccccc3-c3ccccc3-c3ccc4c5ccccc5c5ccccc5c4c3)n2)cc1 ; c1ccc(-c2ccccc2-c2ccc3c4ccccc4c4ccccc4c3c2)c(-c2cc(-c3ccncc3)cc(-c3ccncc3)c2)c1 ; c1cncc(-c2cc(-c3cccnc3)cc(-c3ccccc3-c3ccccc3-c3ccc4c5ccccc5c5ccccc5c4c3)c2)c1]

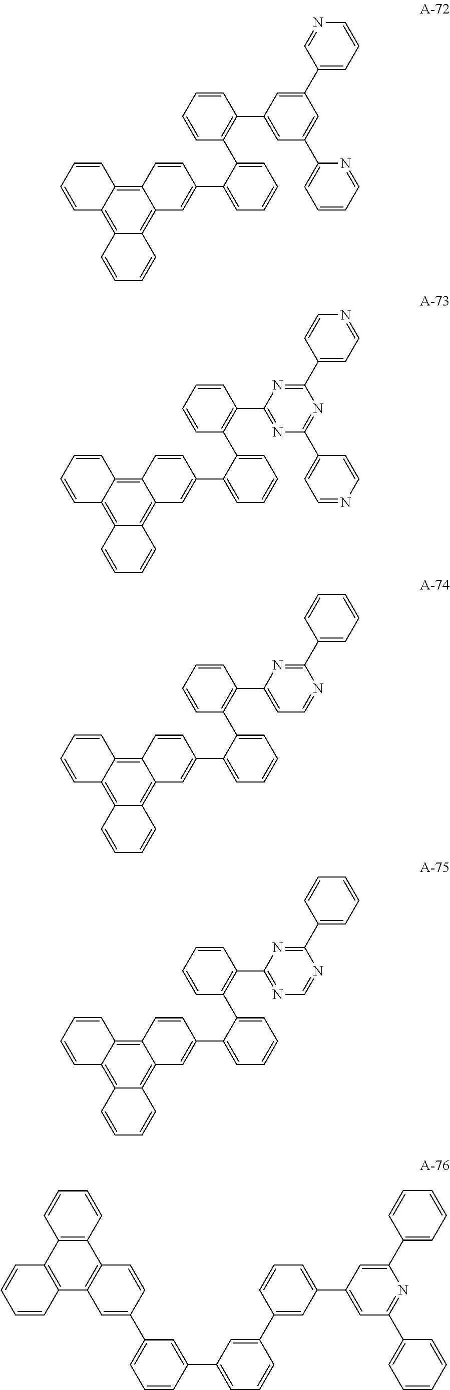 [SMILES: c1ccc(-c2cc(-c3cccnc3)cc(-c3ccccc3-c3ccccc3-c3ccc4c5ccccc5c5ccccc5c4c3)c2)nc1 ; c1ccc(-c2ccccc2-c2nc(-c3ccncc3)nc(-c3ccncc3)n2)c(-c2ccc3c4ccccc4c4ccccc4c3c2)c1 ; c1ccc(-c2nccc(-c3ccccc3-c3ccccc3-c3ccc4c5ccccc5c5ccccc5c4c3)n2)cc1 ; c1ccc(-c2ncnc(-c3ccccc3-c3ccccc3-c3ccc4c5ccccc5c5ccccc5c4c3)n2)cc1 ; c1ccc(-c2cc(-c3cccc(-c4cccc(-c5cccc(-c6ccc7c8ccccc8c8ccccc8c7c6)c5)c4)c3)cc(-c3ccccc3)n2)cc1]

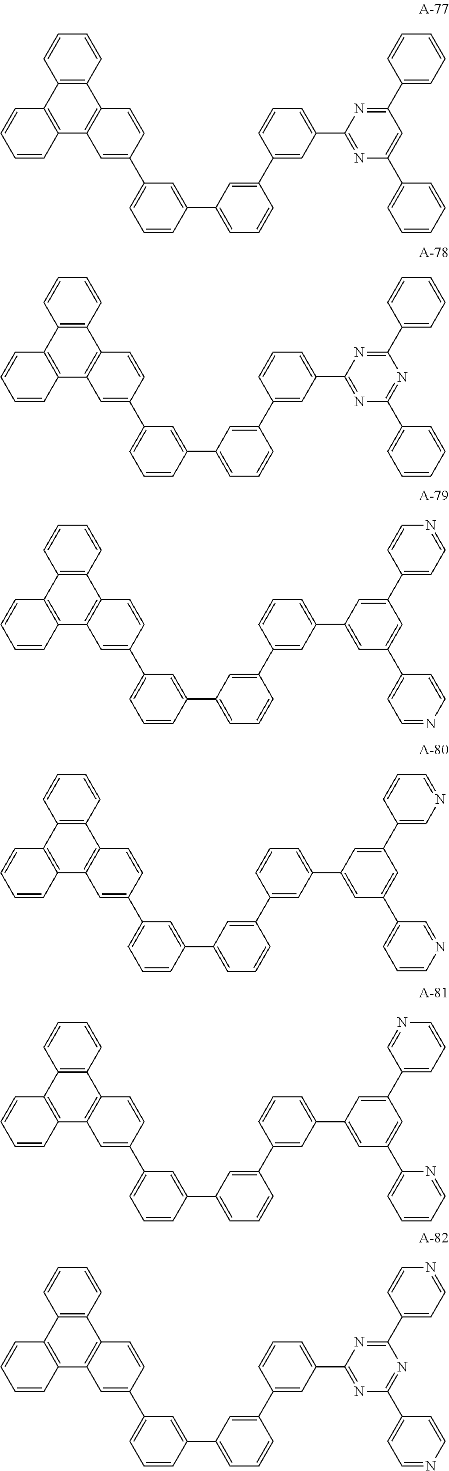

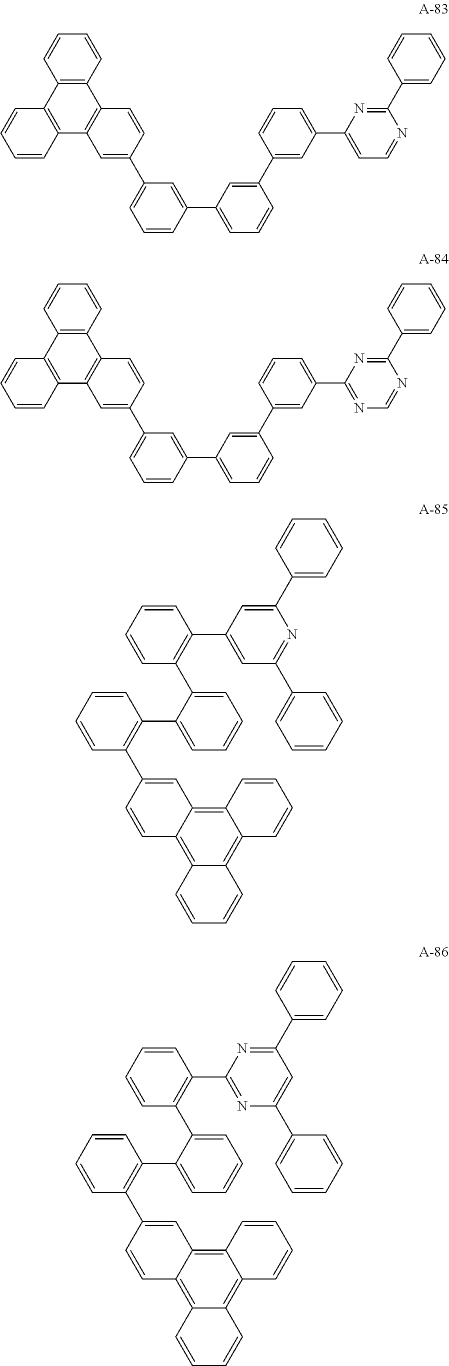 [SMILES: c1ccc(-c2nccc(-c3cccc(-c4cccc(-c5cccc(-c6ccc7c8ccccc8c8ccccc8c7c6)c5)c4)c3)n2)cc1 ; c1ccc(-c2ncnc(-c3cccc(-c4cccc(-c5cccc(-c6ccc7c8ccccc8c8ccccc8c7c6)c5)c4)c3)n2)cc1 ; c1ccc(-c2cc(-c3ccccc3-c3ccccc3-c3ccccc3-c3ccc4c5ccccc5c5ccccc5c4c3)cc(-c3ccccc3)n2)cc1 ; c1ccc(-c2cc(-c3ccccc3)nc(-c3ccccc3-c3ccccc3-c3ccccc3-c3ccc4c5ccccc5c5ccccc5c4c3)n2)cc1]

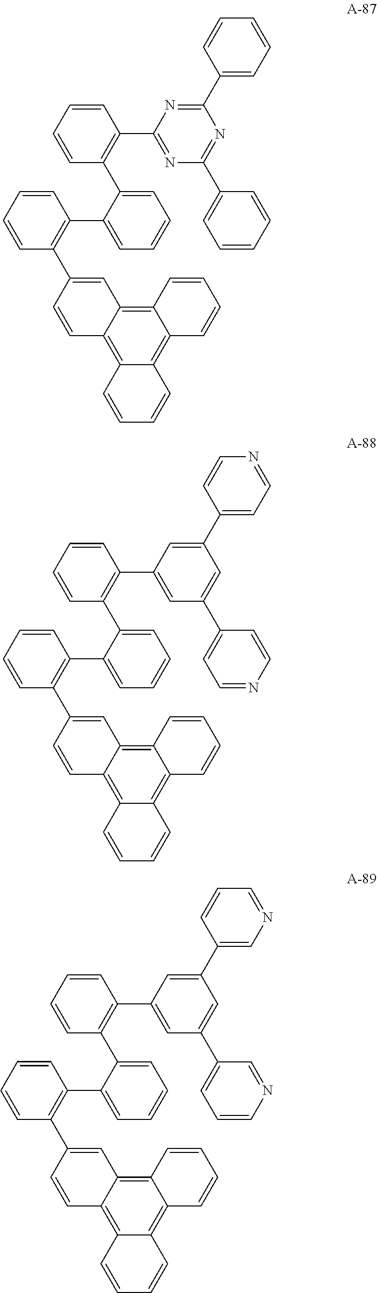 [SMILES: c1ccc(-c2nc(-c3ccccc3)nc(-c3ccccc3-c3ccccc3-c3ccccc3-c3ccc4c5ccccc5c5ccccc5c4c3)n2)cc1 ; c1ccc(-c2ccccc2-c2ccccc2-c2ccc3c4ccccc4c4ccccc4c3c2)c(-c2cc(-c3ccncc3)cc(-c3ccncc3)c2)c1 ; c1cncc(-c2cc(-c3cccnc3)cc(-c3ccccc3-c3ccccc3-c3ccccc3-c3ccc4c5ccccc5c5ccccc5c4c3)c2)c1]

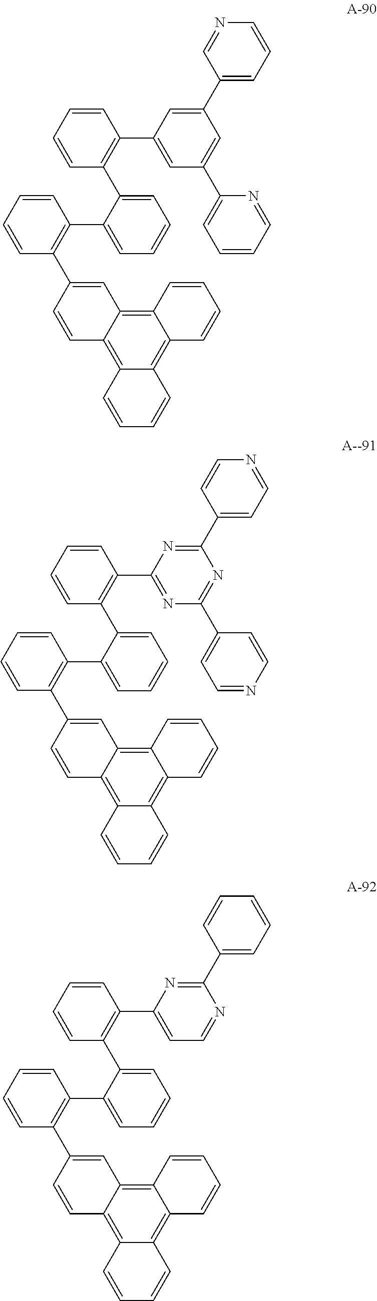 [SMILES: c1ccc(-c2cc(-c3cccnc3)cc(-c3ccccc3-c3ccccc3-c3ccccc3-c3ccc4c5ccccc5c5ccccc5c4c3)c2)nc1 ; c1ccc(-c2ccccc2-c2ccccc2-c2nc(-c3ccncc3)nc(-c3ccncc3)n2)c(-c2ccc3c4ccccc4c4ccccc4c3c2)c1 ; c1ccc(-c2nccc(-c3ccccc3-c3ccccc3-c3ccccc3-c3ccc4c5ccccc5c5ccccc5c4c3)n2)cc1]

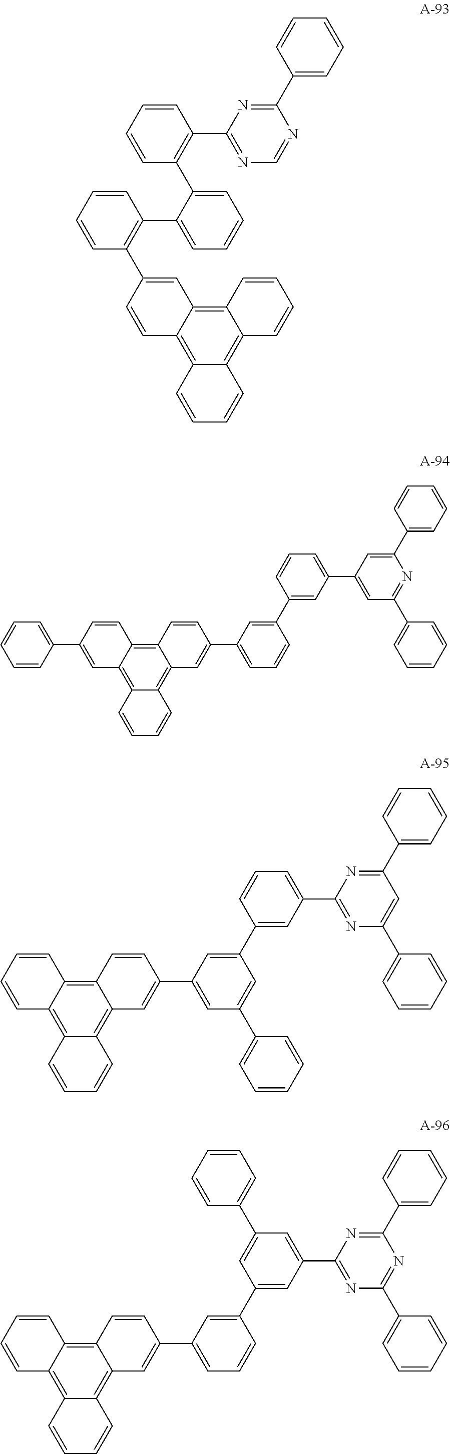 [SMILES: c1ccc(-c2ncnc(-c3ccccc3-c3ccccc3-c3ccccc3-c3ccc4c5ccccc5c5ccccc5c4c3)n2)cc1 ; c1ccc(-c2ccc3c4ccc(-c5cccc(-c6cccc(-c7cc(-c8ccccc8)nc(-c8ccccc8)c7)c6)c5)cc4c4ccccc4c3c2)cc1 ; c1ccc(-c2cc(-c3cccc(-c4nc(-c5ccccc5)cc(-c5ccccc5)n4)c3)cc(-c3ccc4c5ccccc5c5ccccc5c4c3)c2)cc1 ; c1ccc(-c2cc(-c3cccc(-c4ccc5c6ccccc6c6ccccc6c5c4)c3)cc(-c3nc(-c4ccccc4)nc(-c4ccccc4)n3)c2)cc1]

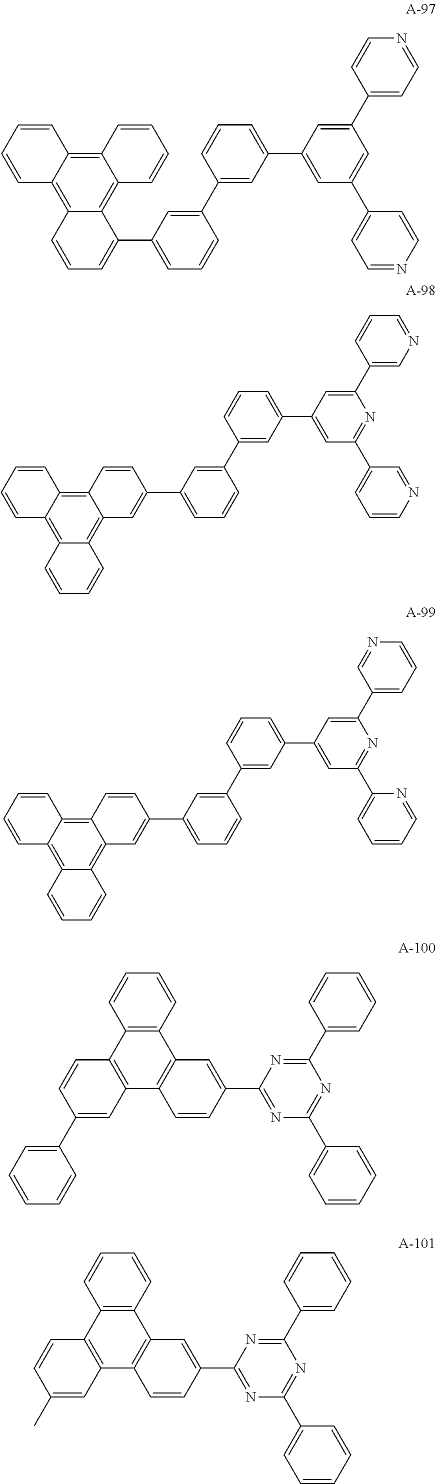

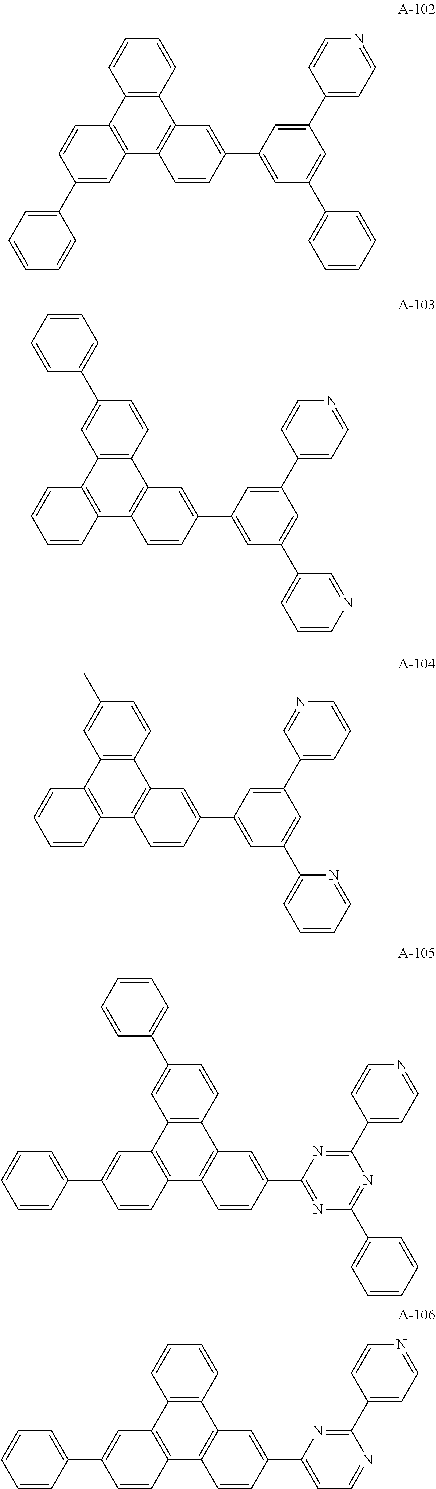

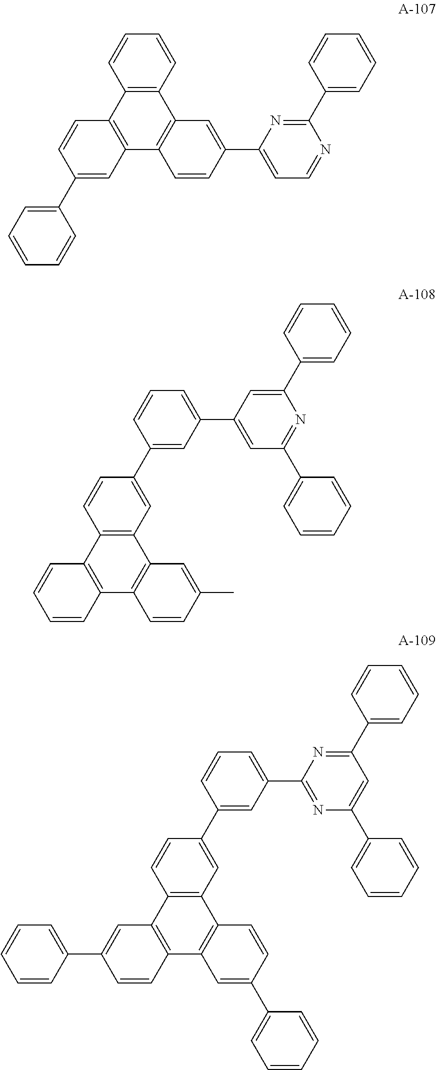 [SMILES: c1ccc(-c2ccc3c4ccccc4c4cc(-c5ccnc(-c6ccccc6)n5)ccc4c3c2)cc1 ; Cc1ccc2c3ccccc3c3ccc(-c4cccc(-c5cc(-c6ccccc6)nc(-c6ccccc6)c5)c4)cc3c2c1 ; c1ccc(-c2ccc3c(c2)c2ccc(-c4cccc(-c5nc(-c6ccccc6)cc(-c6ccccc6)n5)c4)cc2c2ccc(-c4ccccc4)cc32)cc1]

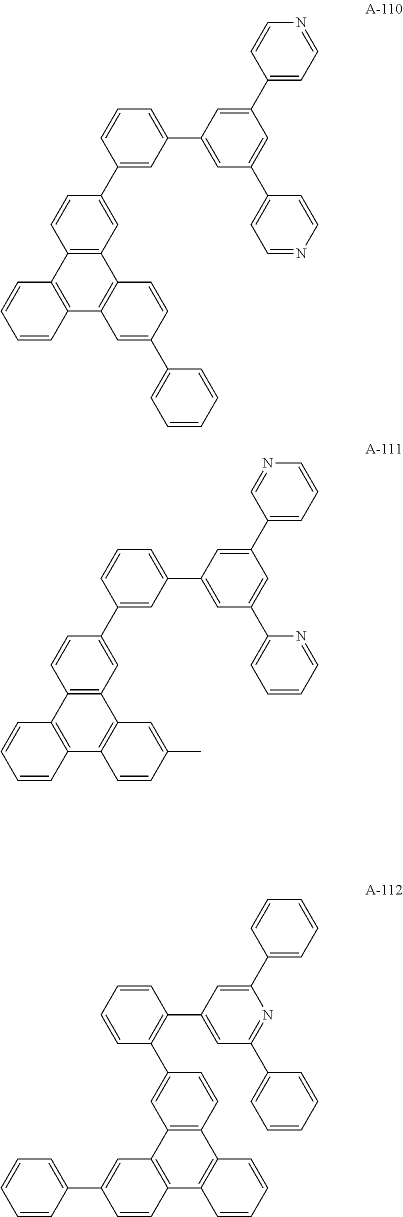 [SMILES: c1ccc(-c2ccc3c(c2)c2ccccc2c2ccc(-c4cccc(-c5cc(-c6ccncc6)cc(-c6ccncc6)c5)c4)cc23)cc1 ; Cc1ccc2c3ccccc3c3ccc(-c4cccc(-c5cc(-c6cccnc6)cc(-c6ccccn6)c5)c4)cc3c2c1 ; c1ccc(-c2ccc3c4ccccc4c4ccc(-c5ccccc5-c5cc(-c6ccccc6)nc(-c6ccccc6)c5)cc4c3c2)cc1]

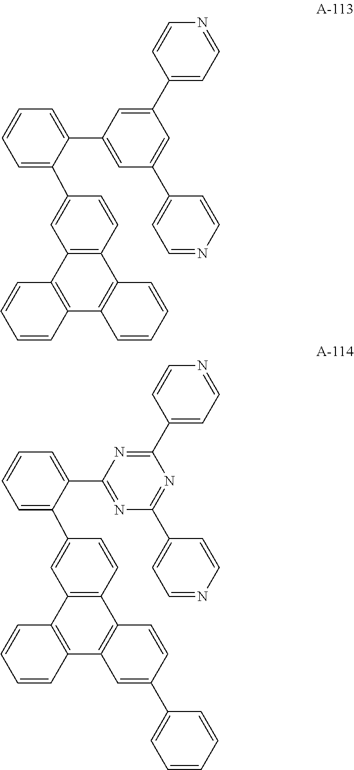

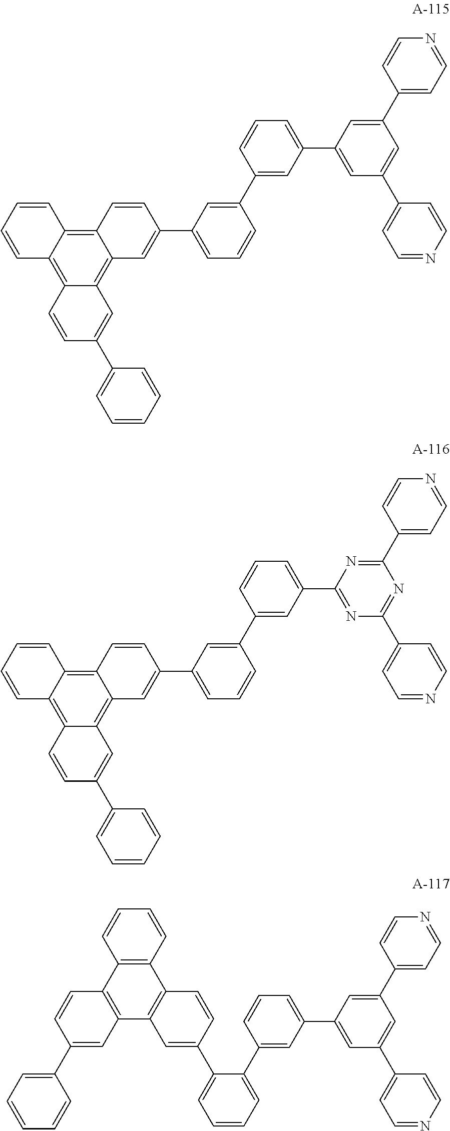 [SMILES: c1ccc(-c2ccc3c4ccccc4c4ccc(-c5cccc(-c6cccc(-c7cc(-c8ccncc8)cc(-c8ccncc8)c7)c6)c5)cc4c3c2)cc1 ; c1ccc(-c2ccc3c4ccccc4c4ccc(-c5cccc(-c6cccc(-c7nc(-c8ccncc8)nc(-c8ccncc8)n7)c6)c5)cc4c3c2)cc1 ; c1ccc(-c2ccc3c4ccccc4c4ccc(-c5ccccc5-c5cccc(-c6cc(-c7ccncc7)cc(-c7ccncc7)c6)c5)cc4c3c2)cc1]

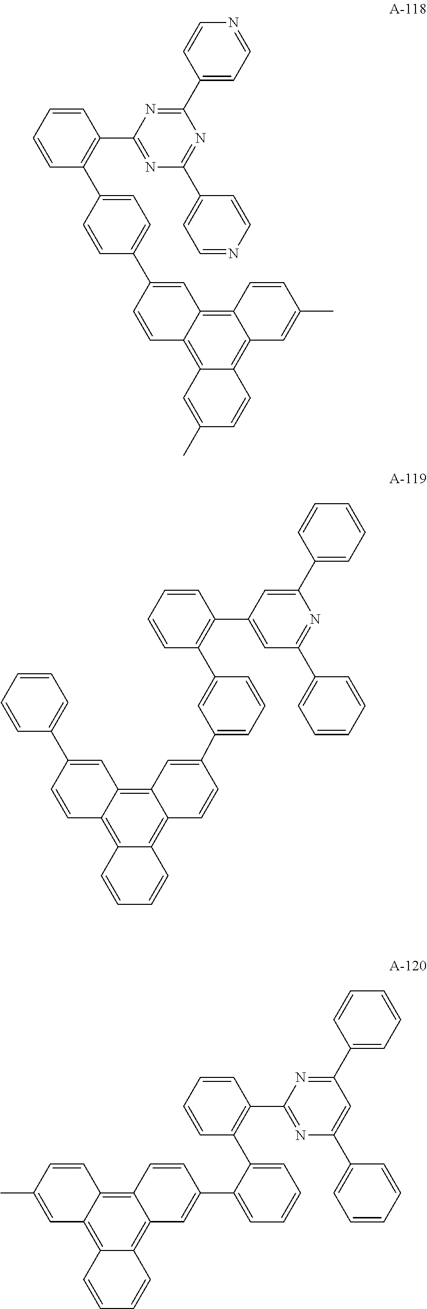

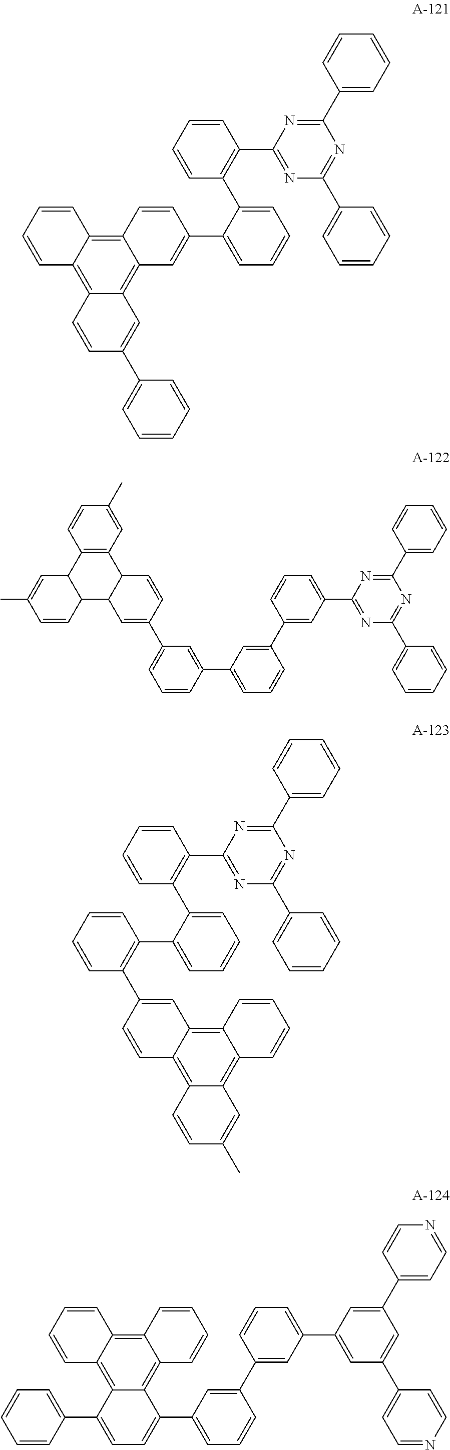 [SMILES: c1ccc(-c2ccc3c4ccccc4c4ccc(-c5ccccc5-c5ccccc5-c5nc(-c6ccccc6)nc(-c6ccccc6)n5)cc4c3c2)cc1 ; CC1=CC2c3ccc(C)cc3C3C=CC(c4cccc(-c5cccc(-c6cccc(-c7nc(-c8ccccc8)nc(-c8ccccc8)n7)c6)c5)c4)=CC3C2C=C1 ; Cc1ccc2c3ccc(-c4ccccc4-c4ccccc4-c4ccccc4-c4nc(-c5ccccc5)nc(-c5ccccc5)n4)cc3c3ccccc3c2c1 ; c1ccc(-c2ccc(-c3cccc(-c4cccc(-c5cc(-c6ccncc6)cc(-c6ccncc6)c5)c4)c3)c3c4ccccc4c4ccccc4c23)cc1]

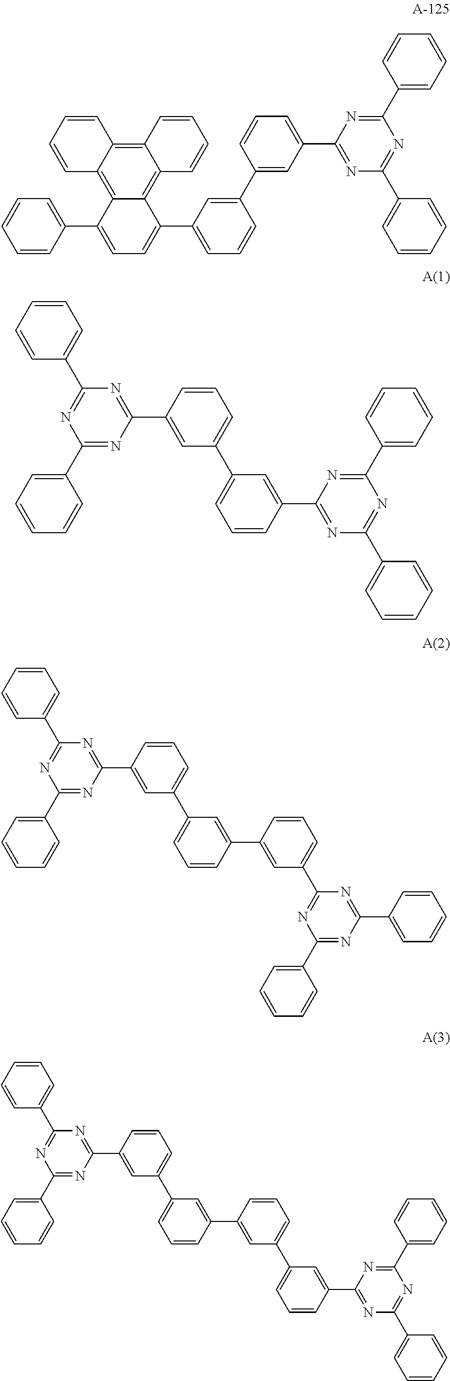 [SMILES: c1ccc(-c2nc(-c3ccccc3)nc(-c3cccc(-c4cccc(-c5ccc(-c6ccccc6)c6c7ccccc7c7ccccc7c56)c4)c3)n2)cc1 ; c1ccc(-c2nc(-c3ccccc3)nc(-c3cccc(-c4cccc(-c5nc(-c6ccccc6)nc(-c6ccccc6)n5)c4)c3)n2)cc1 ; c1ccc(-c2nc(-c3ccccc3)nc(-c3cccc(-c4cccc(-c5cccc(-c6nc(-c7ccccc7)nc(-c7ccccc7)n6)c5)c4)c3)n2)cc1 ; c1ccc(-c2nc(-c3ccccc3)nc(-c3cccc(-c4cccc(-c5cccc(-c6cccc(-c7nc(-c8ccccc8)nc(-c8ccccc8)n7)c6)c5)c4)c3)n2)cc1]

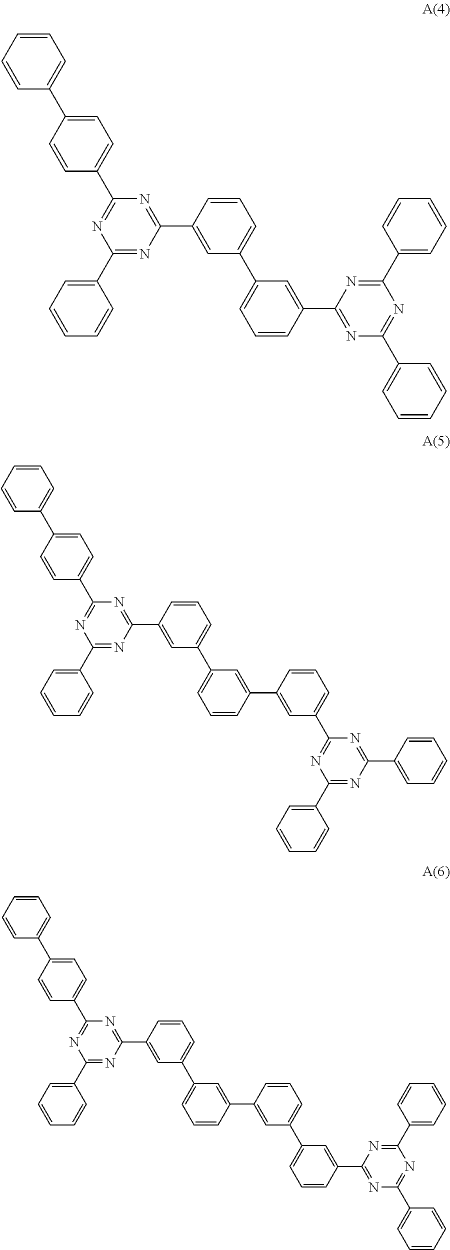 [SMILES: c1ccc(-c2ccc(-c3nc(-c4ccccc4)nc(-c4cccc(-c5cccc(-c6nc(-c7ccccc7)nc(-c7ccccc7)n6)c5)c4)n3)cc2)cc1 ; c1ccc(-c2ccc(-c3nc(-c4ccccc4)nc(-c4cccc(-c5cccc(-c6cccc(-c7nc(-c8ccccc8)nc(-c8ccccc8)n7)c6)c5)c4)n3)cc2)cc1 ; c1ccc(-c2ccc(-c3nc(-c4ccccc4)nc(-c4cccc(-c5cccc(-c6cccc(-c7cccc(-c8nc(-c9ccccc9)nc(-c9ccccc9)n8)c7)c6)c5)c4)n3)cc2)cc1]

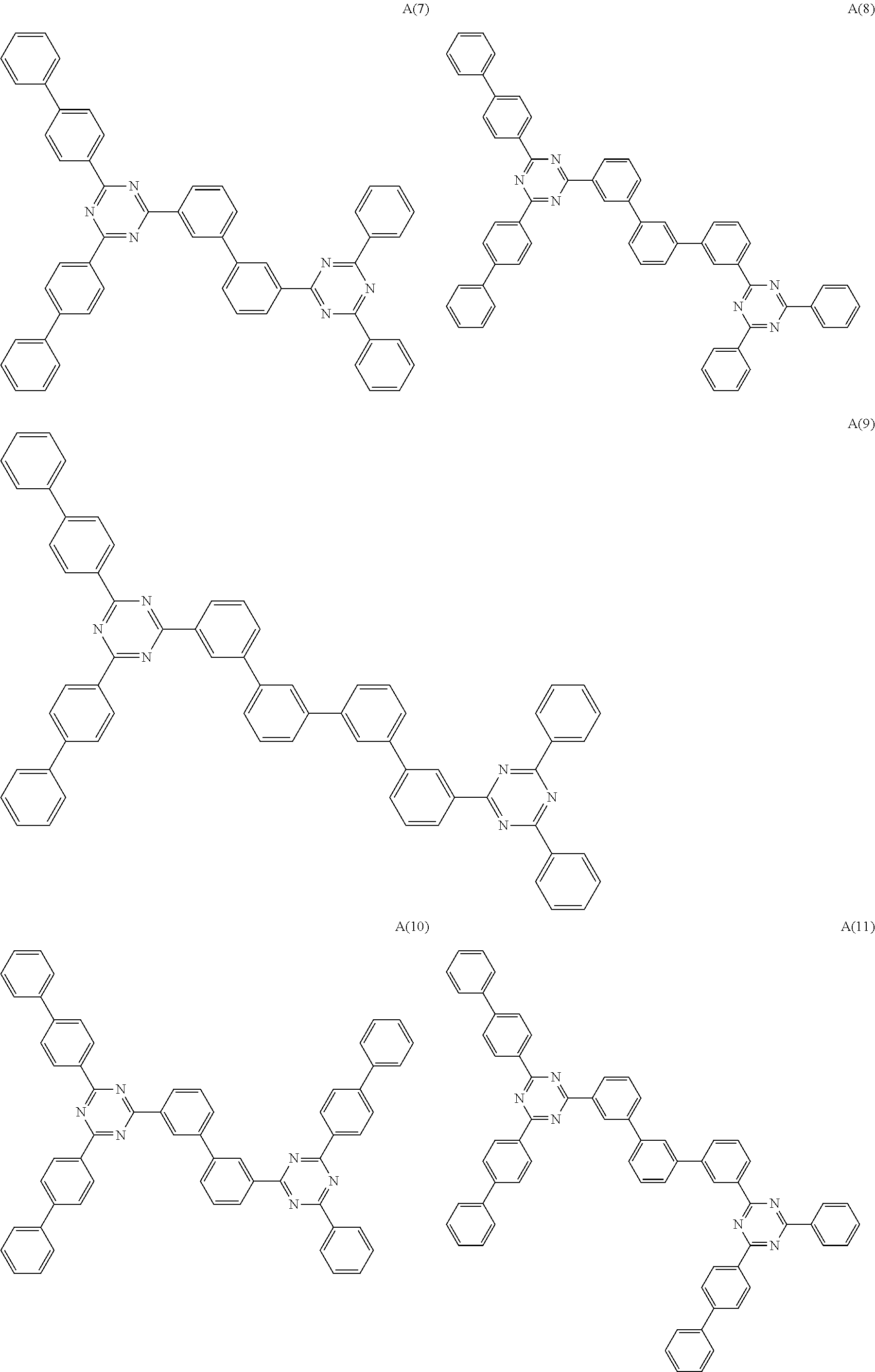

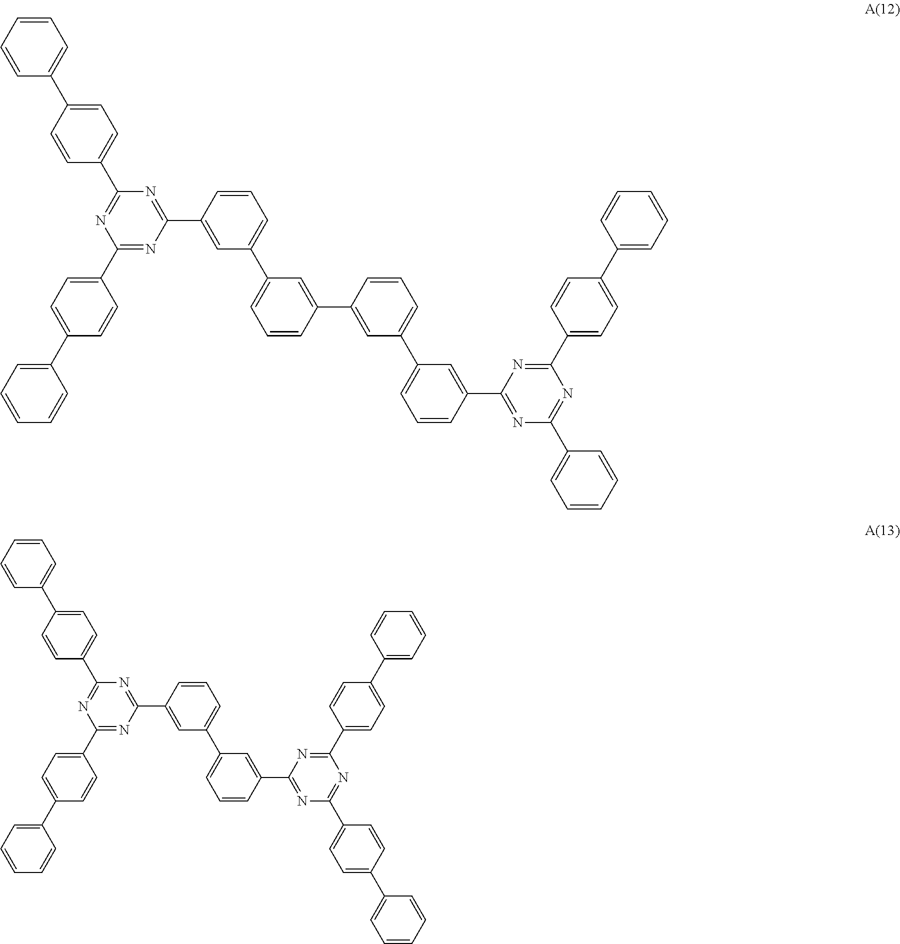 [SMILES: c1ccc(-c2ccc(-c3nc(-c4ccccc4)nc(-c4cccc(-c5cccc(-c6cccc(-c7cccc(-c8nc(-c9ccc(-c%10ccccc%10)cc9)nc(-c9ccc(-c%10ccccc%10)cc9)n8)c7)c6)c5)c4)n3)cc2)cc1 ; c1ccc(-c2ccc(-c3nc(-c4ccc(-c5ccccc5)cc4)nc(-c4cccc(-c5cccc(-c6nc(-c7ccc(-c8ccccc8)cc7)nc(-c7ccc(-c8ccccc8)cc7)n6)c5)c4)n3)cc2)cc1]

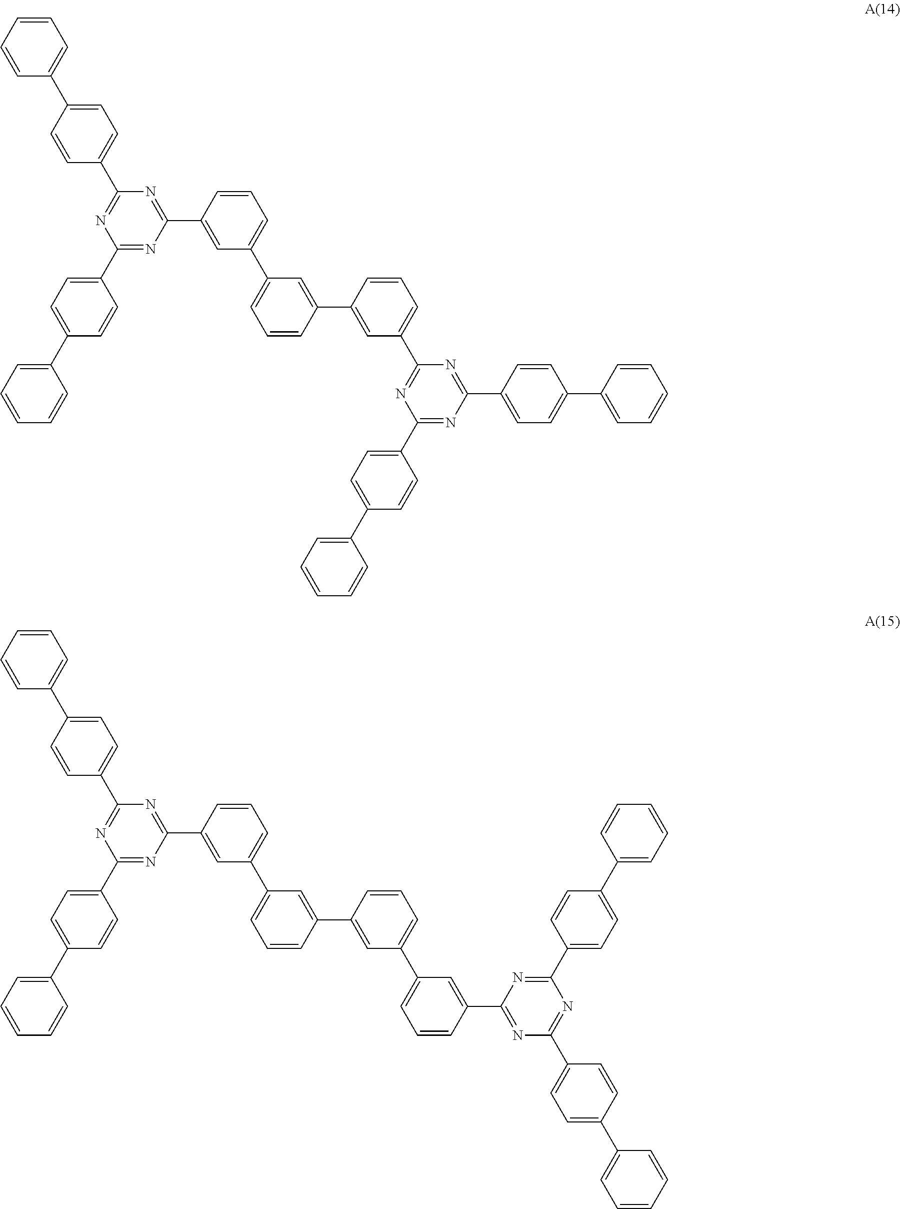 [SMILES: c1ccc(-c2ccc(-c3nc(-c4ccc(-c5ccccc5)cc4)nc(-c4cccc(-c5cccc(-c6cccc(-c7nc(-c8ccc(-c9ccccc9)cc8)nc(-c8ccc(-c9ccccc9)cc8)n7)c6)c5)c4)n3)cc2)cc1 ; c1ccc(-c2ccc(-c3nc(-c4ccc(-c5ccccc5)cc4)nc(-c4cccc(-c5cccc(-c6cccc(-c7cccc(-c8nc(-c9ccc(-c%10ccccc%10)cc9)nc(-c9ccc(-c%10ccccc%10)cc9)n8)c7)c6)c5)c4)n3)cc2)cc1]

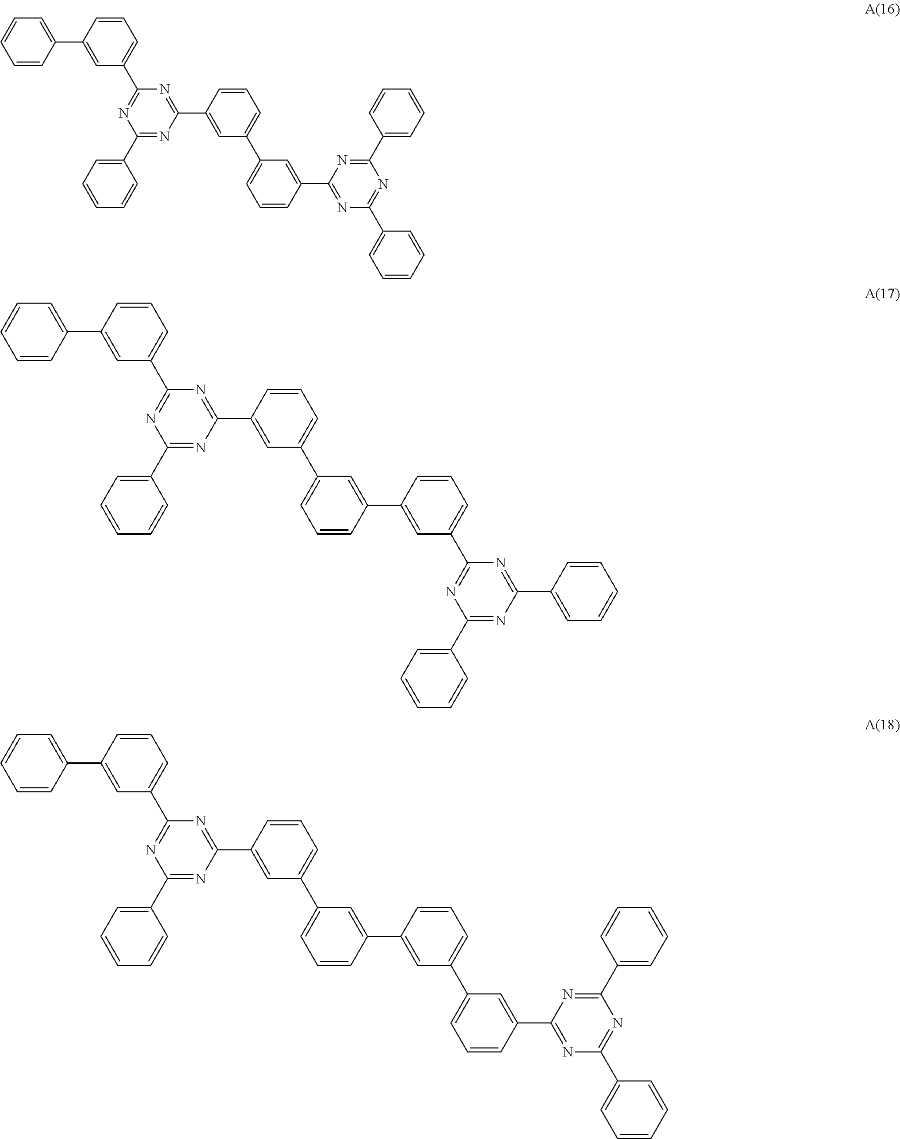 [SMILES: c1ccc(-c2cccc(-c3nc(-c4ccccc4)nc(-c4cccc(-c5cccc(-c6nc(-c7ccccc7)nc(-c7ccccc7)n6)c5)c4)n3)c2)cc1 ; c1ccc(-c2cccc(-c3nc(-c4ccccc4)nc(-c4cccc(-c5cccc(-c6cccc(-c7nc(-c8ccccc8)nc(-c8ccccc8)n7)c6)c5)c4)n3)c2)cc1 ; c1ccc(-c2cccc(-c3nc(-c4ccccc4)nc(-c4cccc(-c5cccc(-c6cccc(-c7cccc(-c8nc(-c9ccccc9)nc(-c9ccccc9)n8)c7)c6)c5)c4)n3)c2)cc1]

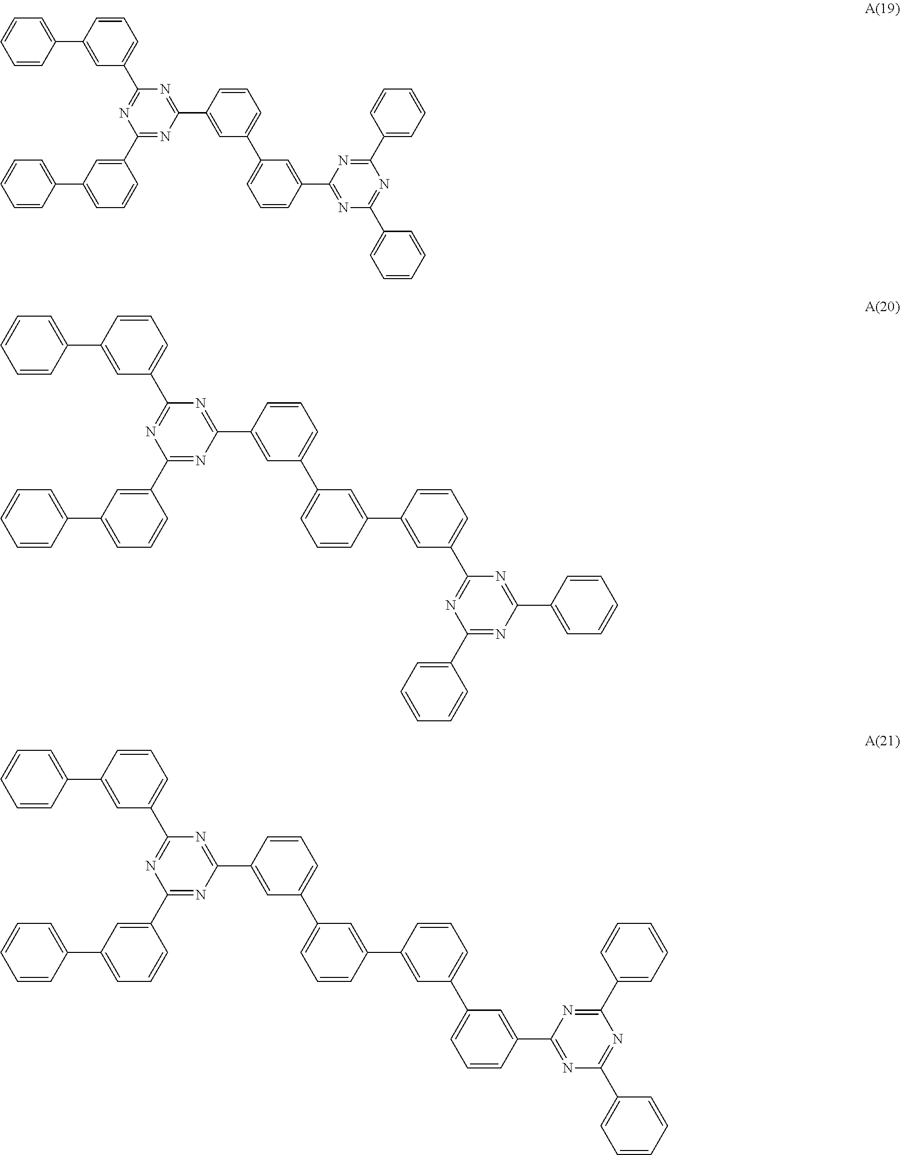 [SMILES: c1ccc(-c2cccc(-c3nc(-c4cccc(-c5ccccc5)c4)nc(-c4cccc(-c5cccc(-c6nc(-c7ccccc7)nc(-c7ccccc7)n6)c5)c4)n3)c2)cc1 ; c1ccc(-c2cccc(-c3nc(-c4cccc(-c5ccccc5)c4)nc(-c4cccc(-c5cccc(-c6cccc(-c7nc(-c8ccccc8)nc(-c8ccccc8)n7)c6)c5)c4)n3)c2)cc1 ; c1ccc(-c2cccc(-c3nc(-c4cccc(-c5ccccc5)c4)nc(-c4cccc(-c5cccc(-c6cccc(-c7cccc(-c8nc(-c9ccccc9)nc(-c9ccccc9)n8)c7)c6)c5)c4)n3)c2)cc1]

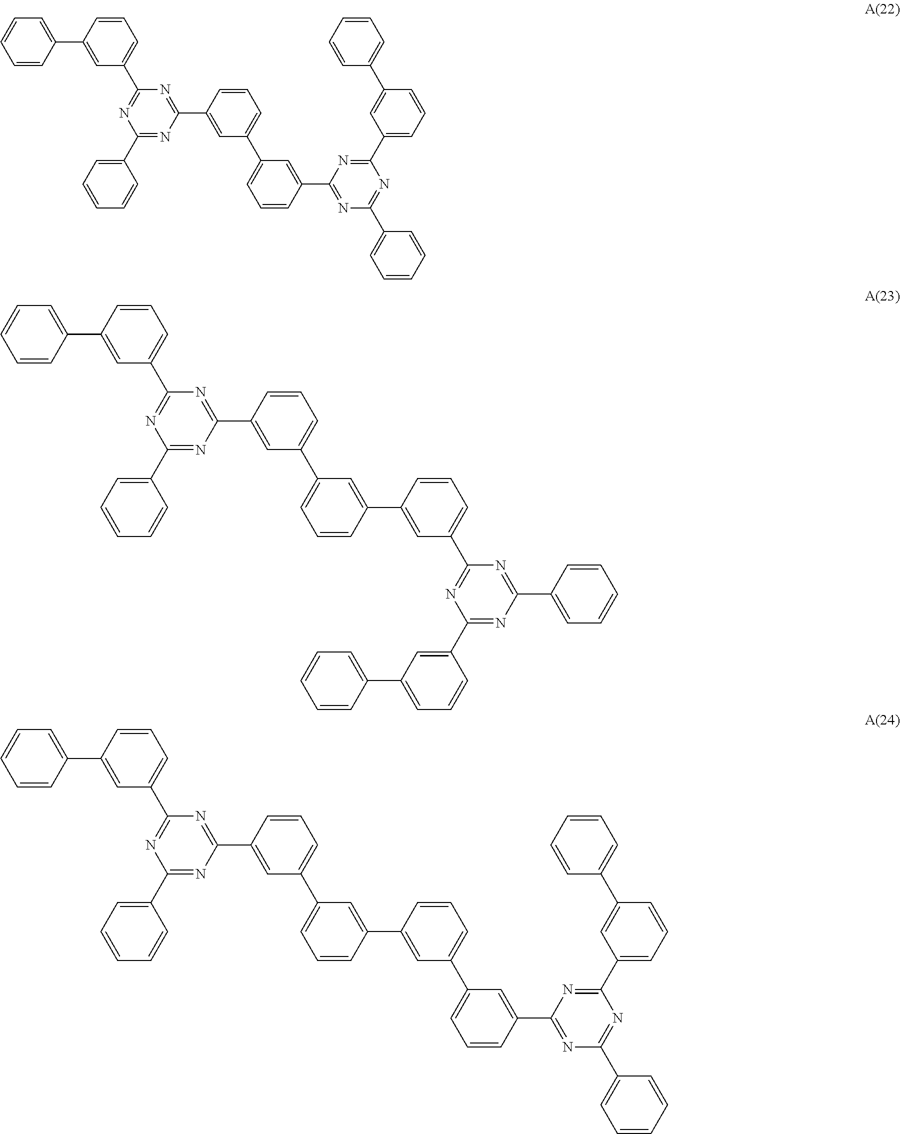 [SMILES: c1ccc(-c2cccc(-c3nc(-c4ccccc4)nc(-c4cccc(-c5cccc(-c6nc(-c7ccccc7)nc(-c7cccc(-c8ccccc8)c7)n6)c5)c4)n3)c2)cc1 ; c1ccc(-c2cccc(-c3nc(-c4ccccc4)nc(-c4cccc(-c5cccc(-c6cccc(-c7nc(-c8ccccc8)nc(-c8cccc(-c9ccccc9)c8)n7)c6)c5)c4)n3)c2)cc1 ; c1ccc(-c2cccc(-c3nc(-c4ccccc4)nc(-c4cccc(-c5cccc(-c6cccc(-c7cccc(-c8nc(-c9ccccc9)nc(-c9cccc(-c%10ccccc%10)c9)n8)c7)c6)c5)c4)n3)c2)cc1]

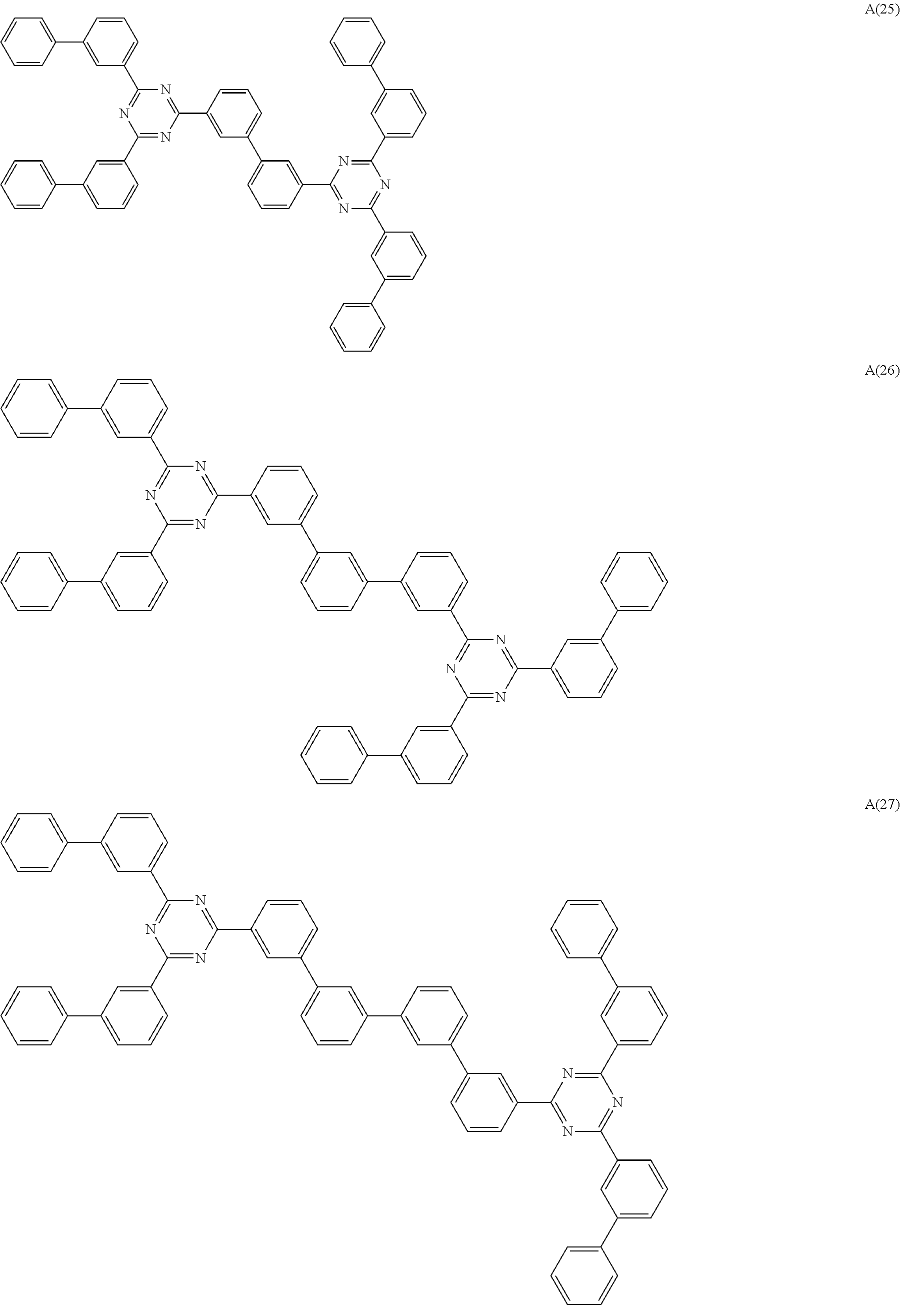 [SMILES: c1ccc(-c2cccc(-c3nc(-c4cccc(-c5ccccc5)c4)nc(-c4cccc(-c5cccc(-c6nc(-c7cccc(-c8ccccc8)c7)nc(-c7cccc(-c8ccccc8)c7)n6)c5)c4)n3)c2)cc1 ; c1ccc(-c2cccc(-c3nc(-c4cccc(-c5ccccc5)c4)nc(-c4cccc(-c5cccc(-c6cccc(-c7nc(-c8cccc(-c9ccccc9)c8)nc(-c8cccc(-c9ccccc9)c8)n7)c6)c5)c4)n3)c2)cc1 ; c1ccc(-c2cccc(-c3nc(-c4cccc(-c5ccccc5)c4)nc(-c4cccc(-c5cccc(-c6cccc(-c7cccc(-c8nc(-c9cccc(-c%10ccccc%10)c9)nc(-c9cccc(-c%10ccccc%10)c9)n8)c7)c6)c5)c4)n3)c2)cc1]

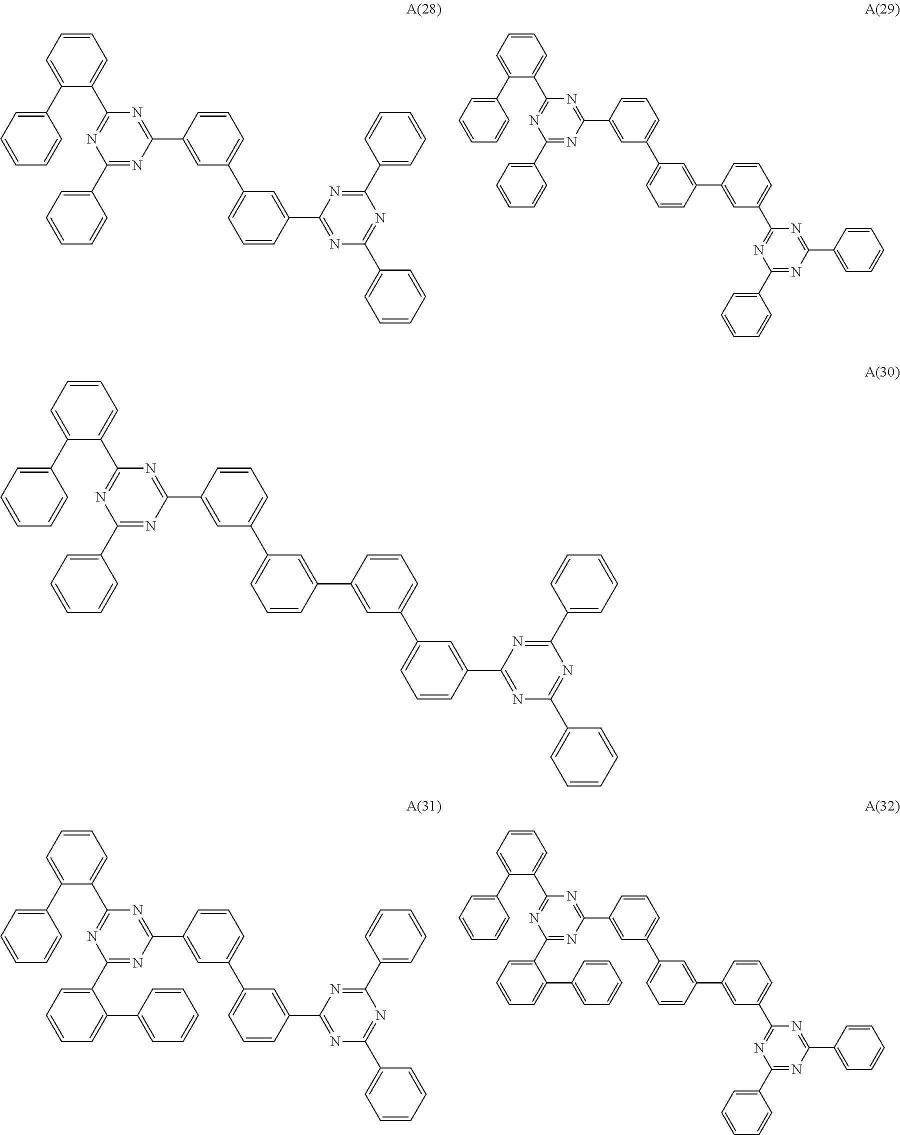

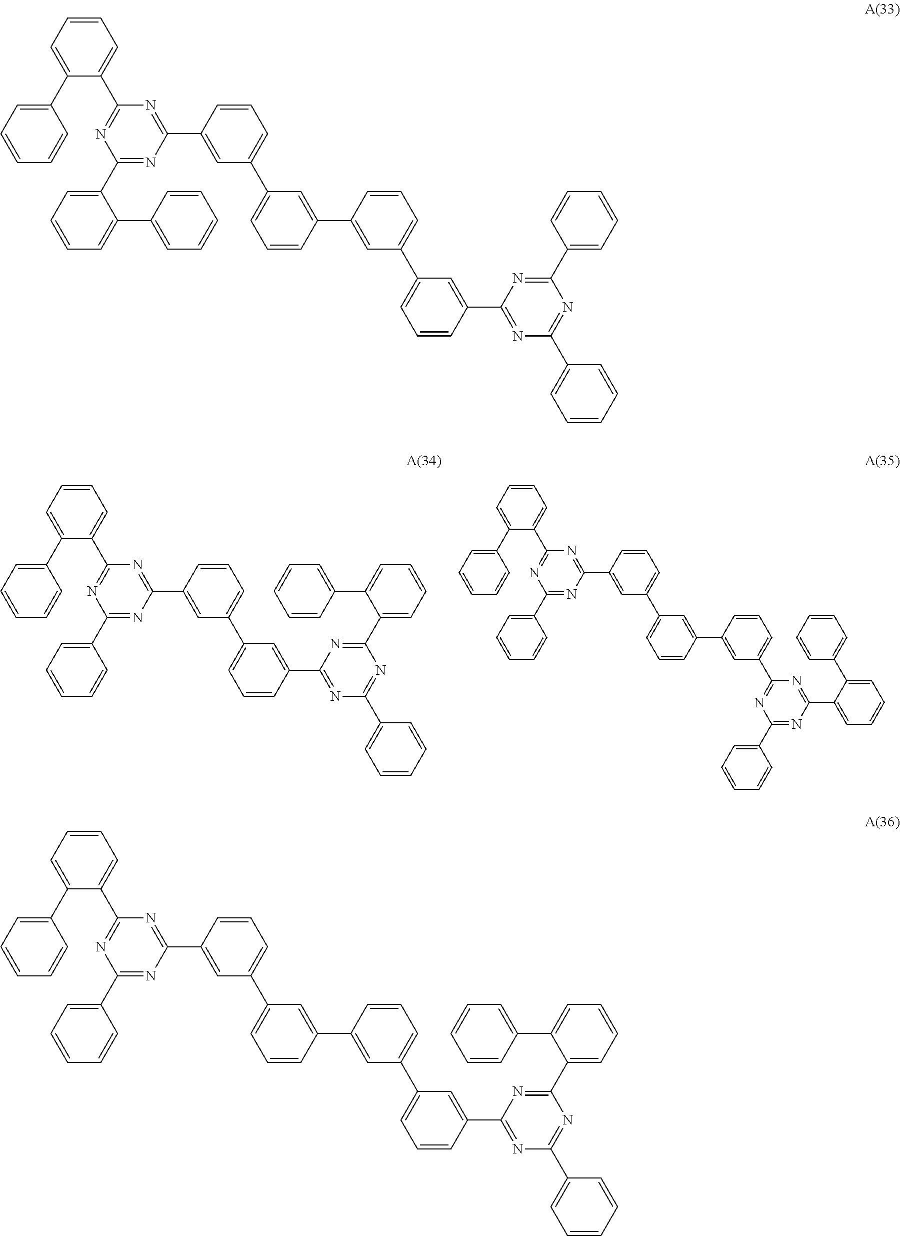

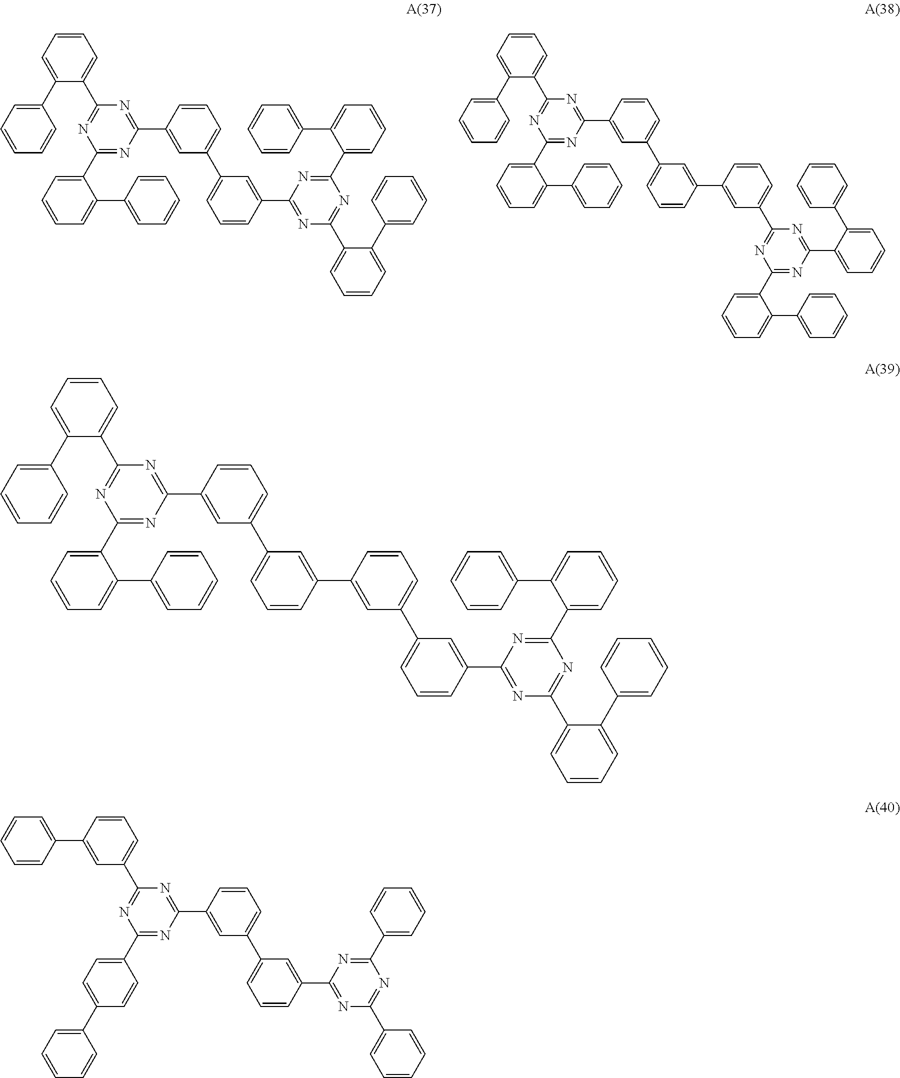

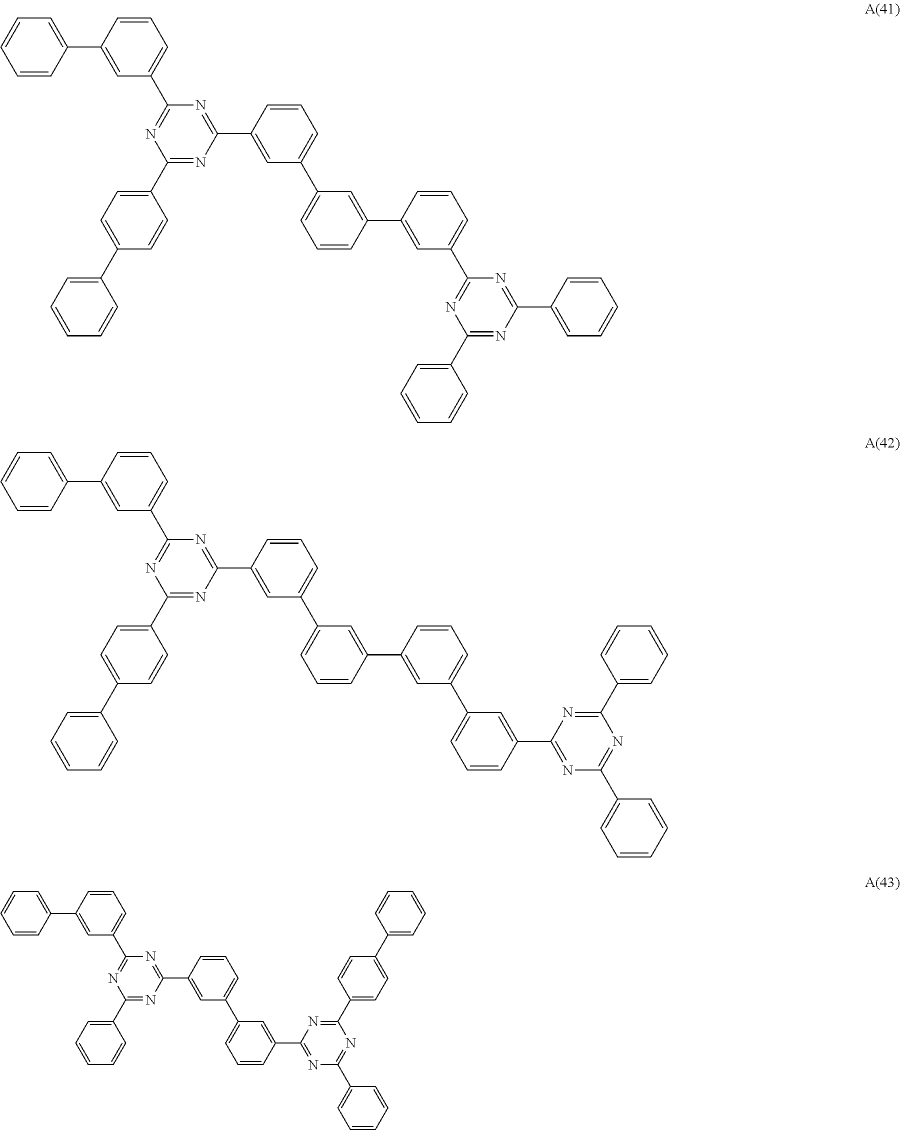 [SMILES: c1ccc(-c2ccc(-c3nc(-c4cccc(-c5ccccc5)c4)nc(-c4cccc(-c5cccc(-c6cccc(-c7nc(-c8ccccc8)nc(-c8ccccc8)n7)c6)c5)c4)n3)cc2)cc1 ; c1ccc(-c2ccc(-c3nc(-c4cccc(-c5ccccc5)c4)nc(-c4cccc(-c5cccc(-c6cccc(-c7cccc(-c8nc(-c9ccccc9)nc(-c9ccccc9)n8)c7)c6)c5)c4)n3)cc2)cc1 ; c1ccc(-c2ccc(-c3nc(-c4ccccc4)nc(-c4cccc(-c5cccc(-c6nc(-c7ccccc7)nc(-c7cccc(-c8ccccc8)c7)n6)c5)c4)n3)cc2)cc1]

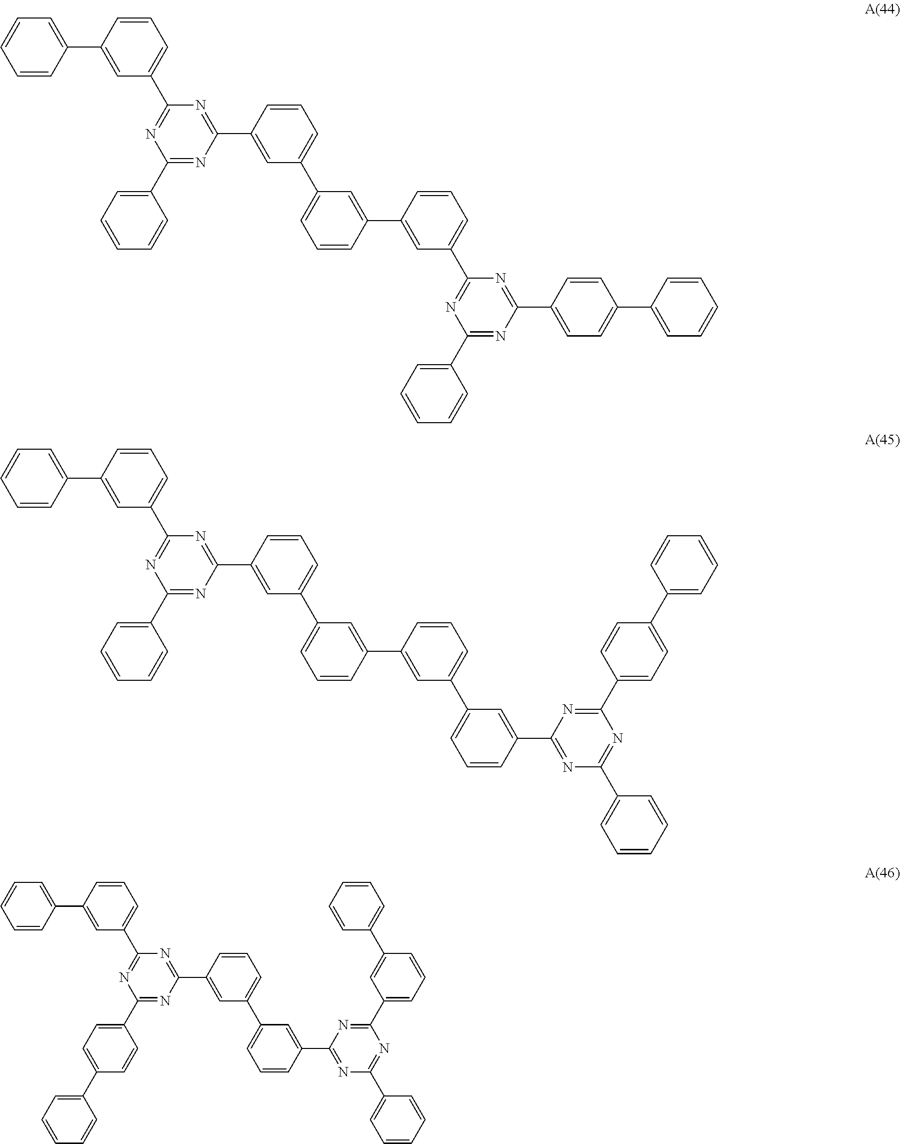 [SMILES: c1ccc(-c2ccc(-c3nc(-c4ccccc4)nc(-c4cccc(-c5cccc(-c6cccc(-c7nc(-c8ccccc8)nc(-c8cccc(-c9ccccc9)c8)n7)c6)c5)c4)n3)cc2)cc1 ; c1ccc(-c2ccc(-c3nc(-c4ccccc4)nc(-c4cccc(-c5cccc(-c6cccc(-c7cccc(-c8nc(-c9ccccc9)nc(-c9cccc(-c%10ccccc%10)c9)n8)c7)c6)c5)c4)n3)cc2)cc1 ; c1ccc(-c2ccc(-c3nc(-c4cccc(-c5ccccc5)c4)nc(-c4cccc(-c5cccc(-c6nc(-c7ccccc7)nc(-c7cccc(-c8ccccc8)c7)n6)c5)c4)n3)cc2)cc1]

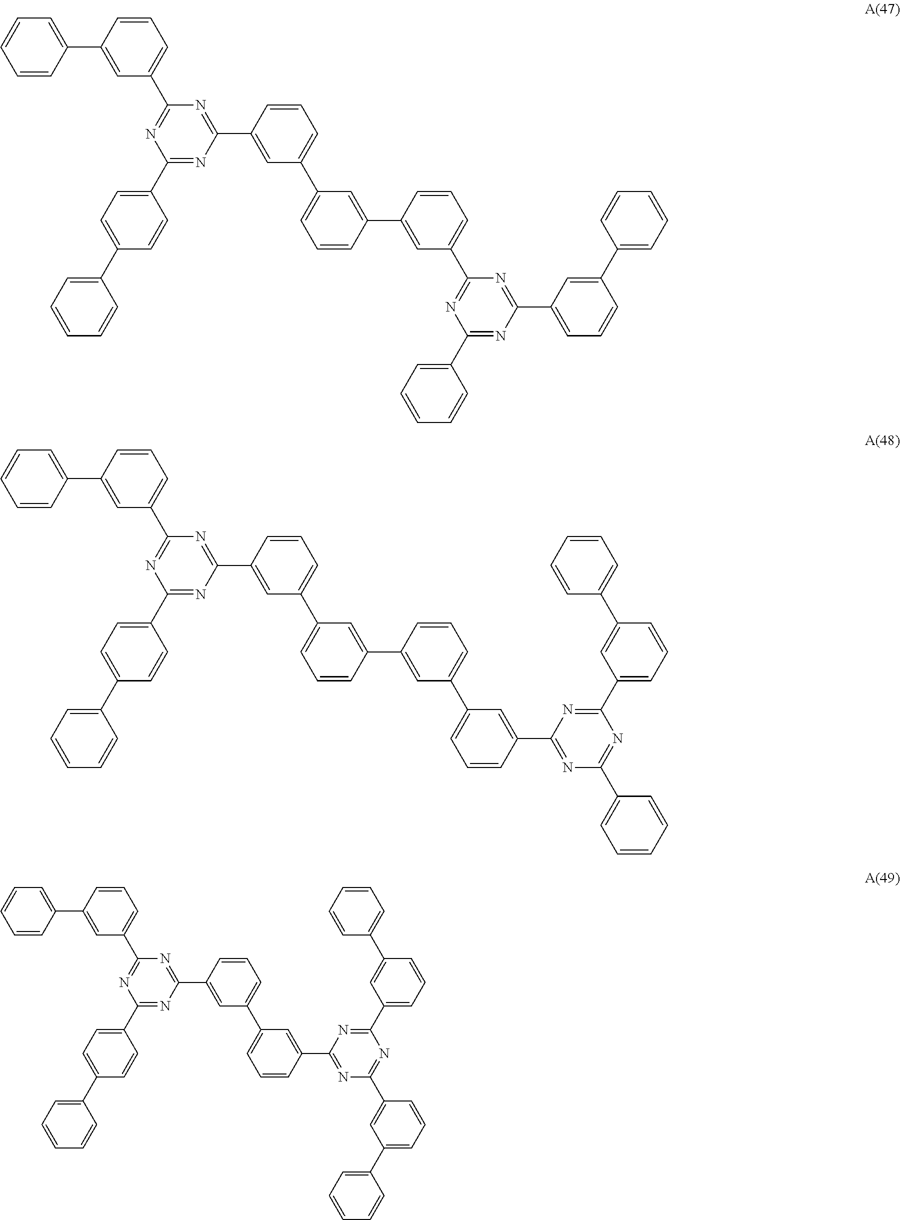 [SMILES: c1ccc(-c2ccc(-c3nc(-c4cccc(-c5ccccc5)c4)nc(-c4cccc(-c5cccc(-c6cccc(-c7nc(-c8ccccc8)nc(-c8cccc(-c9ccccc9)c8)n7)c6)c5)c4)n3)cc2)cc1 ; c1ccc(-c2ccc(-c3nc(-c4cccc(-c5ccccc5)c4)nc(-c4cccc(-c5cccc(-c6cccc(-c7cccc(-c8nc(-c9ccccc9)nc(-c9cccc(-c%10ccccc%10)c9)n8)c7)c6)c5)c4)n3)cc2)cc1 ; c1ccc(-c2ccc(-c3nc(-c4cccc(-c5ccccc5)c4)nc(-c4cccc(-c5cccc(-c6nc(-c7cccc(-c8ccccc8)c7)nc(-c7cccc(-c8ccccc8)c7)n6)c5)c4)n3)cc2)cc1]

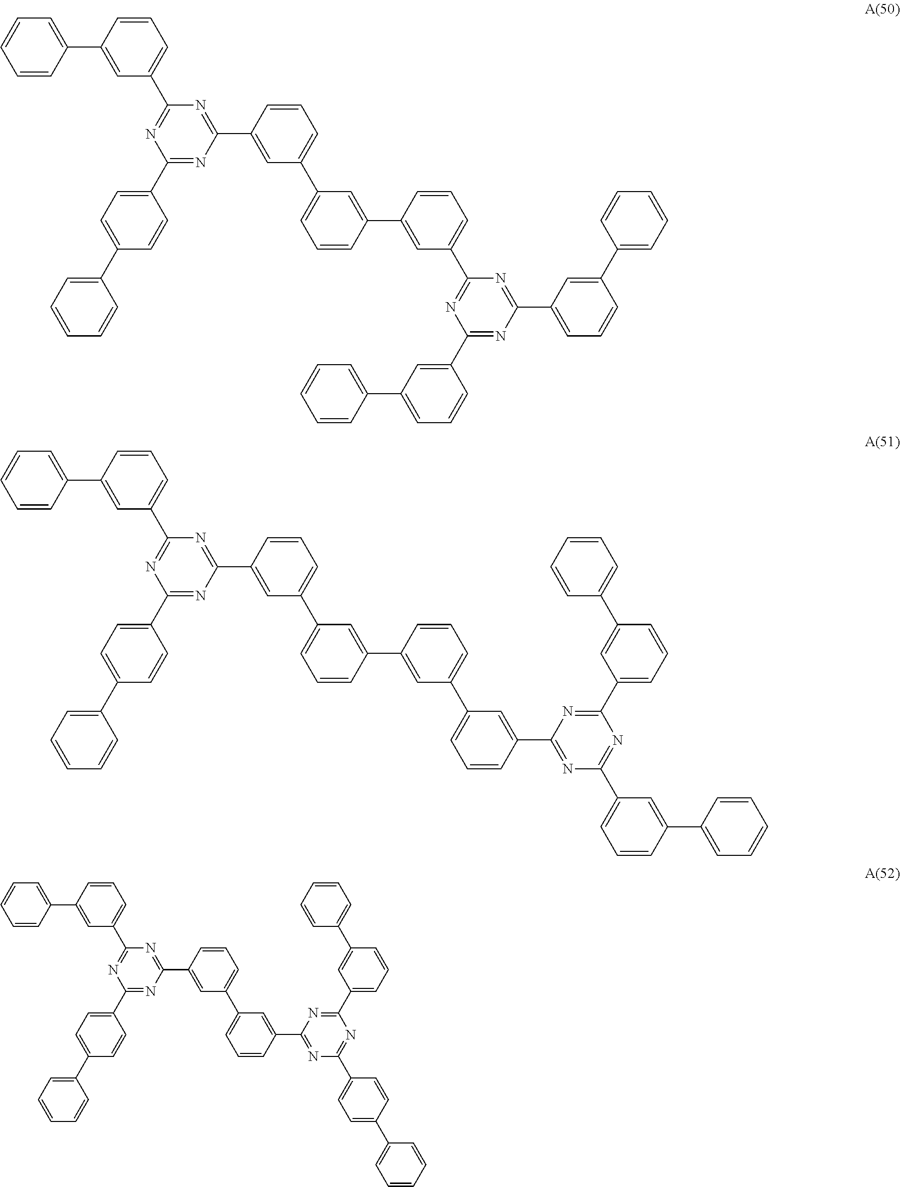 [SMILES: c1ccc(-c2ccc(-c3nc(-c4cccc(-c5ccccc5)c4)nc(-c4cccc(-c5cccc(-c6cccc(-c7nc(-c8cccc(-c9ccccc9)c8)nc(-c8cccc(-c9ccccc9)c8)n7)c6)c5)c4)n3)cc2)cc1 ; c1ccc(-c2ccc(-c3nc(-c4cccc(-c5ccccc5)c4)nc(-c4cccc(-c5cccc(-c6cccc(-c7cccc(-c8nc(-c9cccc(-c%10ccccc%10)c9)nc(-c9cccc(-c%10ccccc%10)c9)n8)c7)c6)c5)c4)n3)cc2)cc1 ; c1ccc(-c2ccc(-c3nc(-c4cccc(-c5ccccc5)c4)nc(-c4cccc(-c5cccc(-c6nc(-c7ccc(-c8ccccc8)cc7)nc(-c7cccc(-c8ccccc8)c7)n6)c5)c4)n3)cc2)cc1]

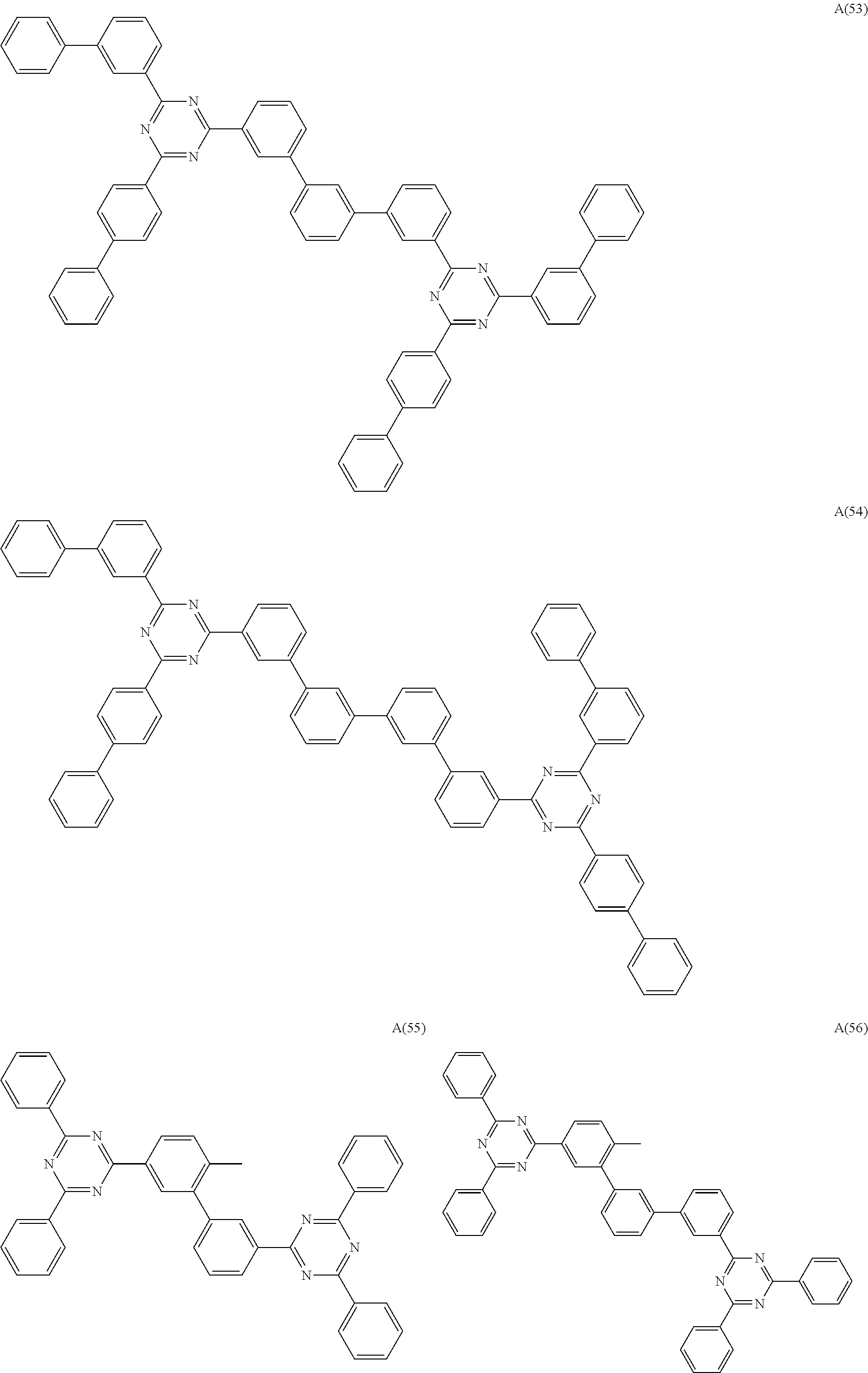 [SMILES: c1ccc(-c2ccc(-c3nc(-c4cccc(-c5ccccc5)c4)nc(-c4cccc(-c5cccc(-c6cccc(-c7nc(-c8ccc(-c9ccccc9)cc8)nc(-c8cccc(-c9ccccc9)c8)n7)c6)c5)c4)n3)cc2)cc1 ; c1ccc(-c2ccc(-c3nc(-c4cccc(-c5ccccc5)c4)nc(-c4cccc(-c5cccc(-c6cccc(-c7cccc(-c8nc(-c9ccc(-c%10ccccc%10)cc9)nc(-c9cccc(-c%10ccccc%10)c9)n8)c7)c6)c5)c4)n3)cc2)cc1 ; Cc1ccc(-c2nc(-c3ccccc3)nc(-c3ccccc3)n2)cc1-c1cccc(-c2nc(-c3ccccc3)nc(-c3ccccc3)n2)c1 ; Cc1ccc(-c2nc(-c3ccccc3)nc(-c3ccccc3)n2)cc1-c1cccc(-c2cccc(-c3nc(-c4ccccc4)nc(-c4ccccc4)n3)c2)c1]

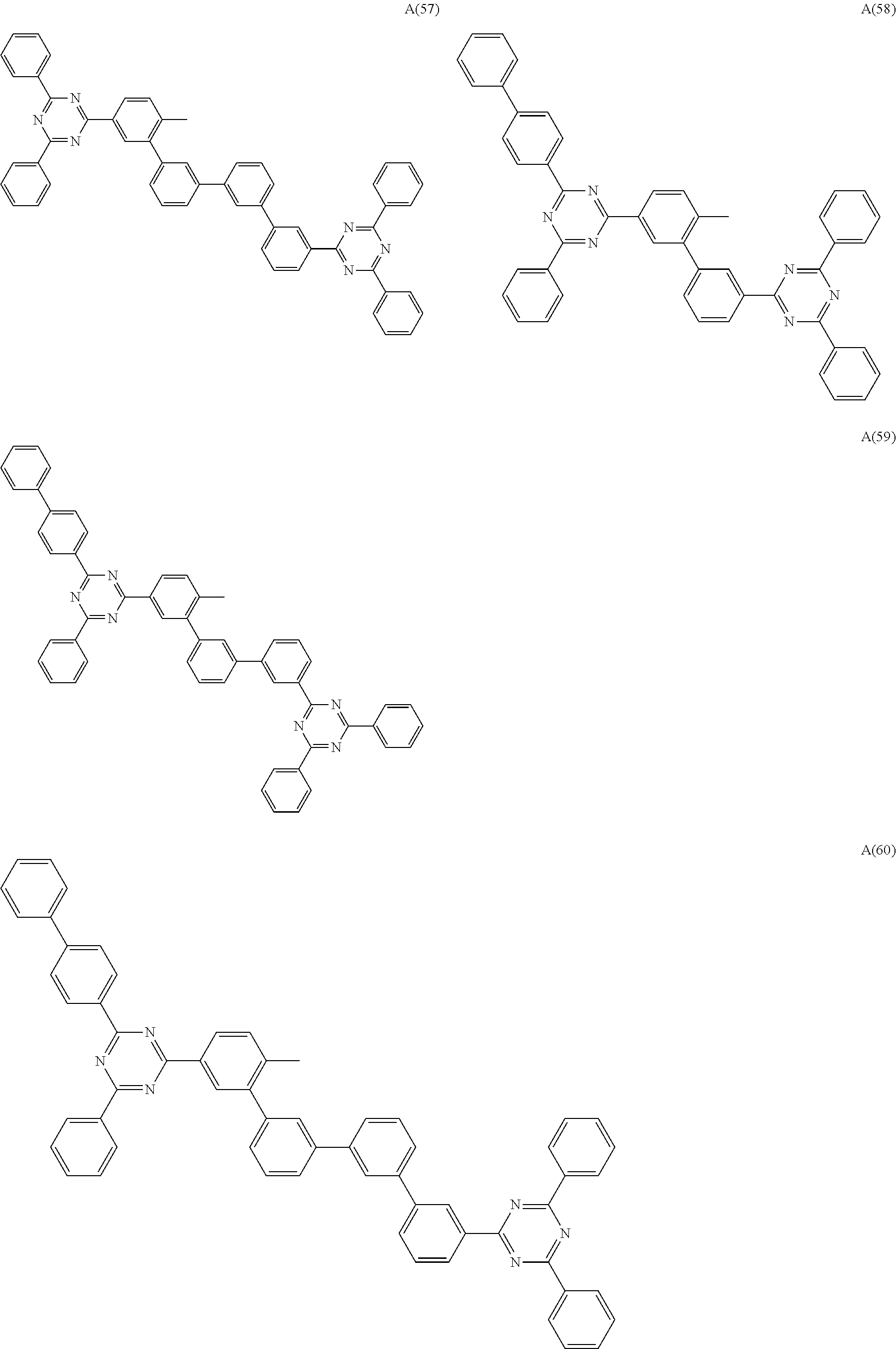 [SMILES: Cc1ccc(-c2nc(-c3ccccc3)nc(-c3ccccc3)n2)cc1-c1cccc(-c2cccc(-c3cccc(-c4nc(-c5ccccc5)nc(-c5ccccc5)n4)c3)c2)c1 ; Cc1ccc(-c2nc(-c3ccccc3)nc(-c3ccc(-c4ccccc4)cc3)n2)cc1-c1cccc(-c2nc(-c3ccccc3)nc(-c3ccccc3)n2)c1 ; Cc1ccc(-c2nc(-c3ccccc3)nc(-c3ccc(-c4ccccc4)cc3)n2)cc1-c1cccc(-c2cccc(-c3nc(-c4ccccc4)nc(-c4ccccc4)n3)c2)c1 ; Cc1ccc(-c2nc(-c3ccccc3)nc(-c3ccc(-c4ccccc4)cc3)n2)cc1-c1cccc(-c2cccc(-c3cccc(-c4nc(-c5ccccc5)nc(-c5ccccc5)n4)c3)c2)c1]

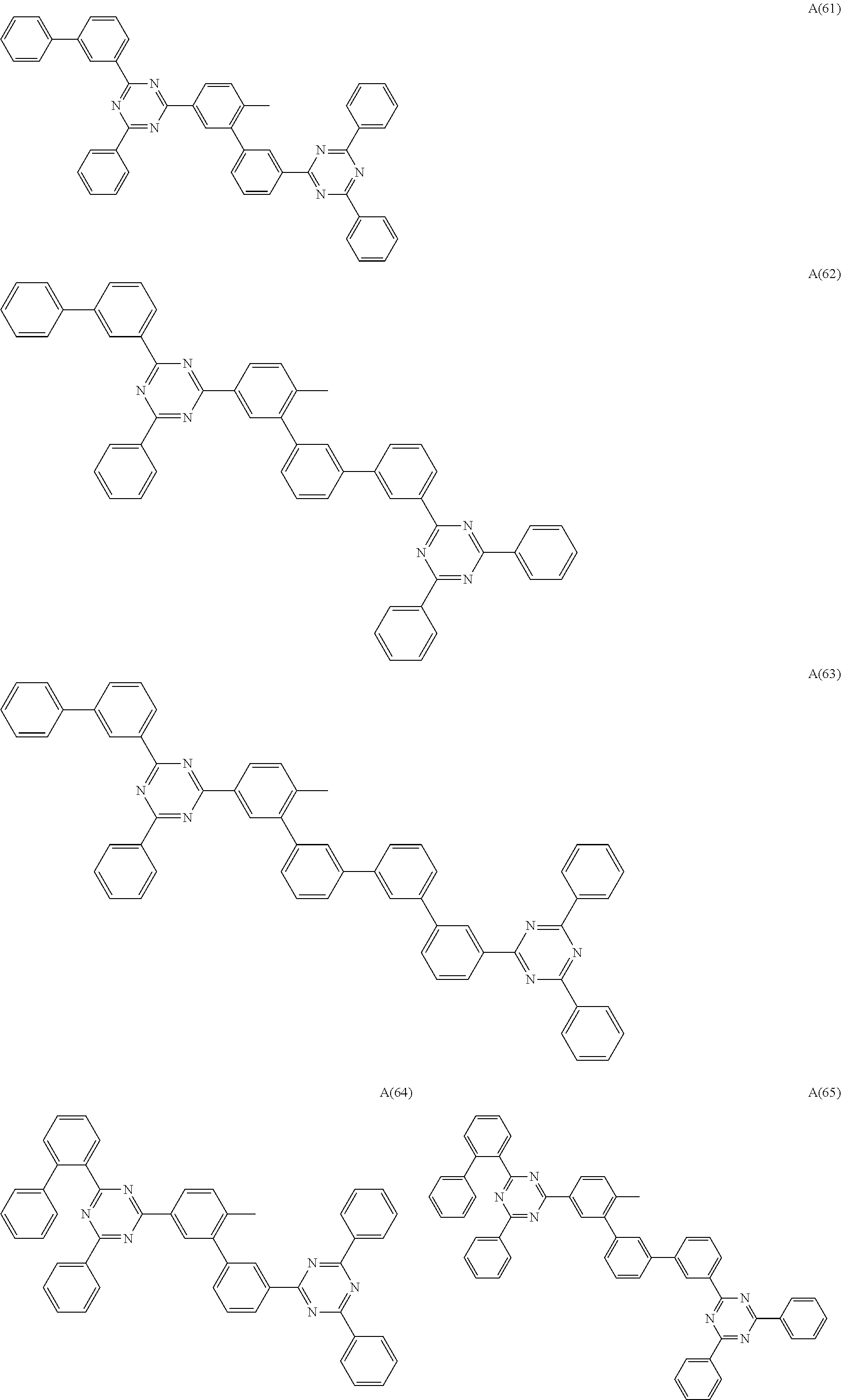

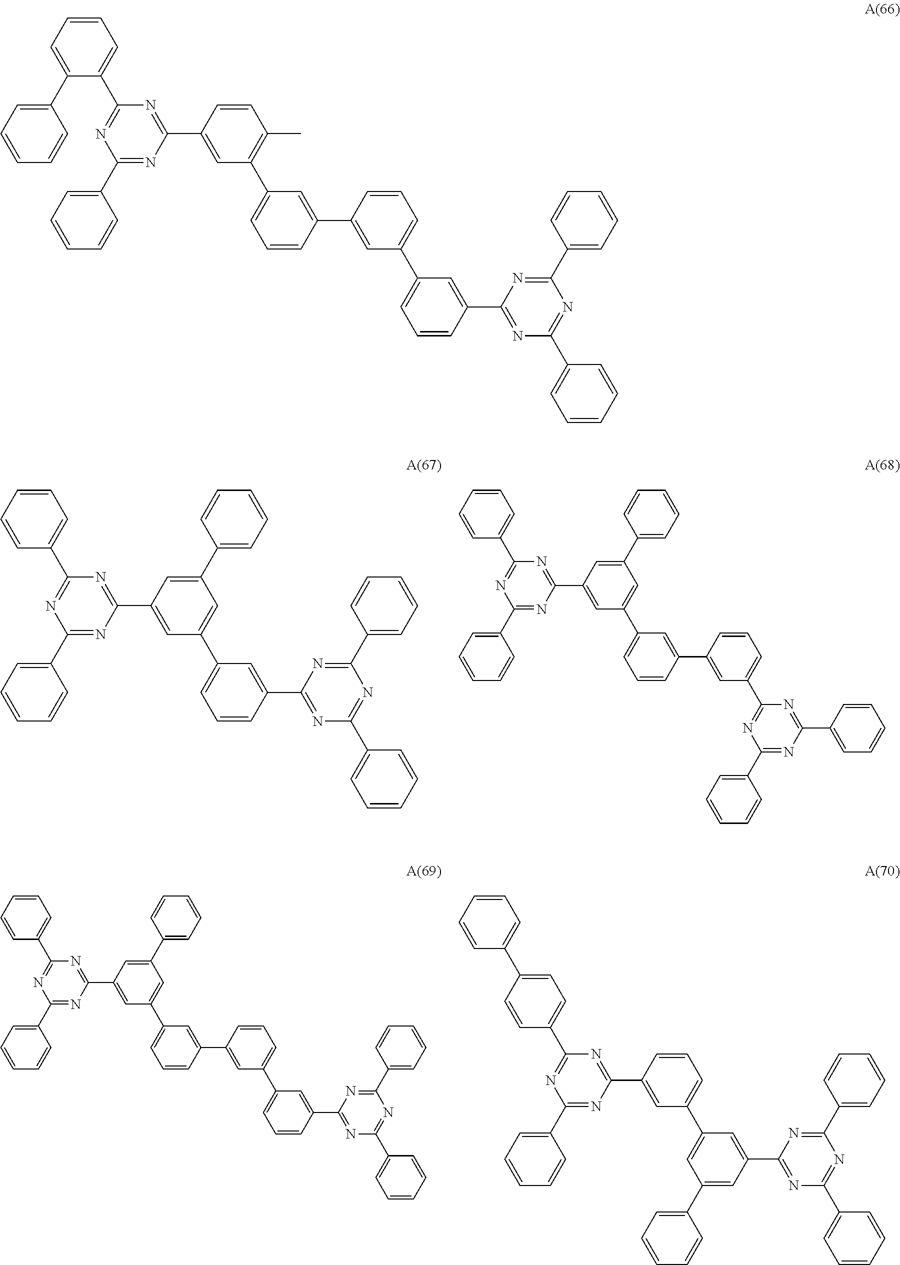

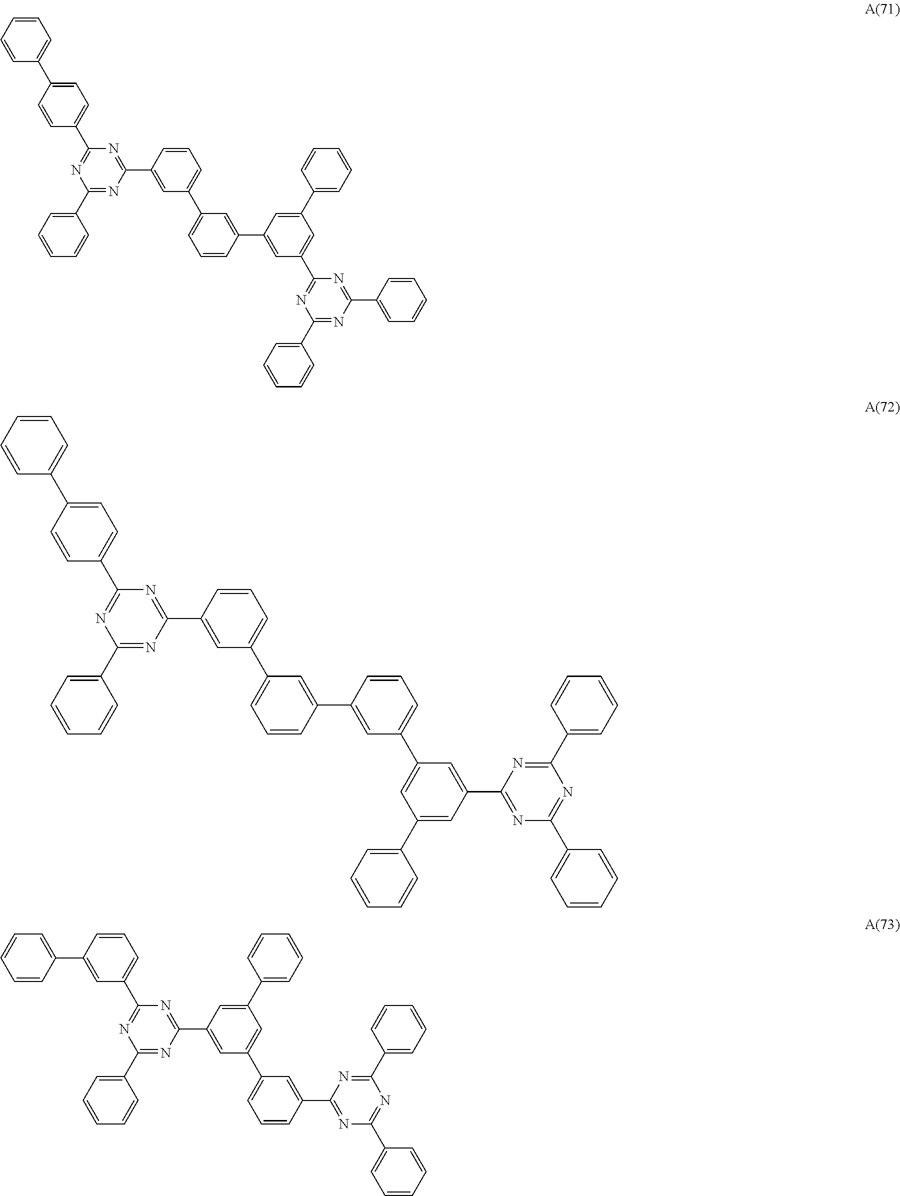 [SMILES: c1ccc(-c2ccc(-c3nc(-c4ccccc4)nc(-c4cccc(-c5cccc(-c6cc(-c7ccccc7)cc(-c7nc(-c8ccccc8)nc(-c8ccccc8)n7)c6)c5)c4)n3)cc2)cc1 ; c1ccc(-c2ccc(-c3nc(-c4ccccc4)nc(-c4cccc(-c5cccc(-c6cccc(-c7cc(-c8ccccc8)cc(-c8nc(-c9ccccc9)nc(-c9ccccc9)n8)c7)c6)c5)c4)n3)cc2)cc1 ; c1ccc(-c2cccc(-c3nc(-c4ccccc4)nc(-c4cc(-c5ccccc5)cc(-c5cccc(-c6nc(-c7ccccc7)nc(-c7ccccc7)n6)c5)c4)n3)c2)cc1]

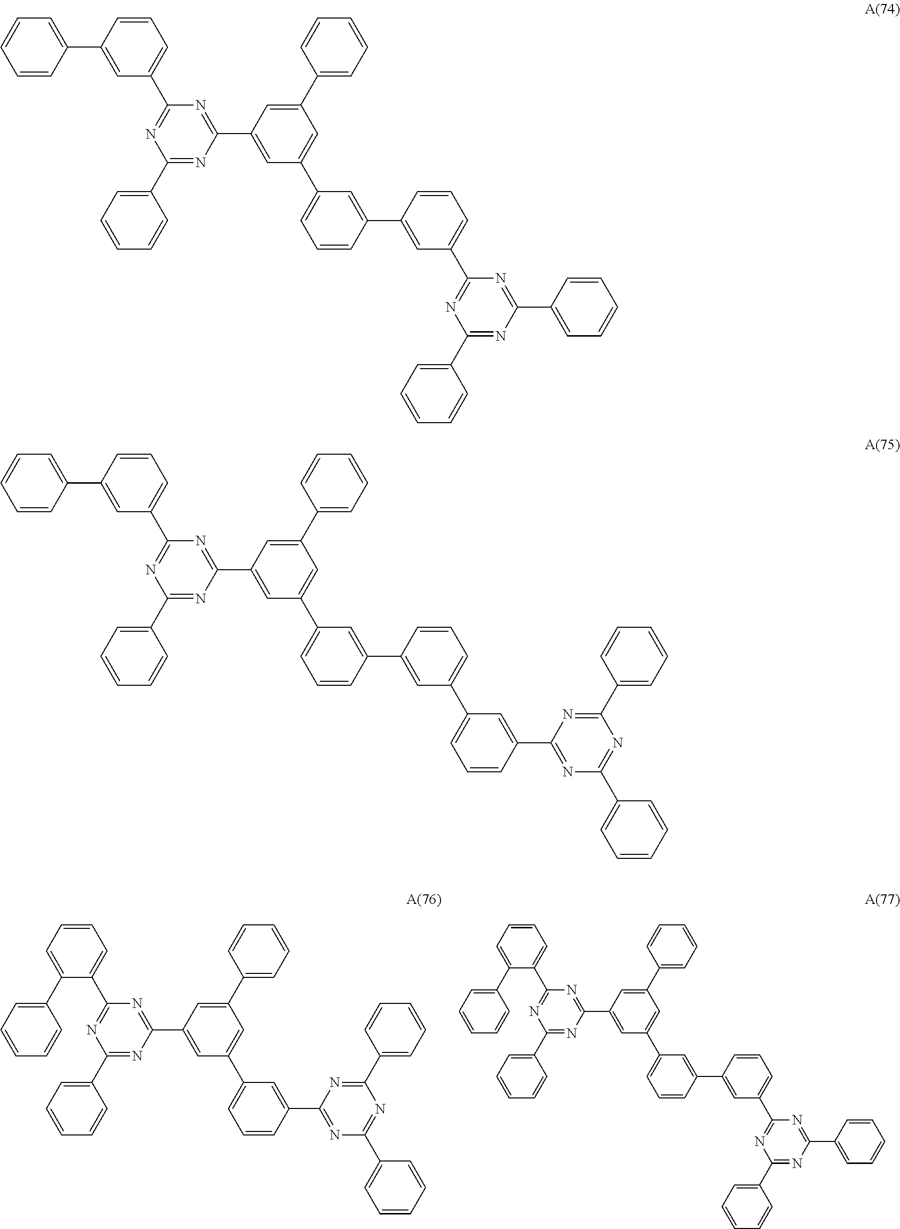

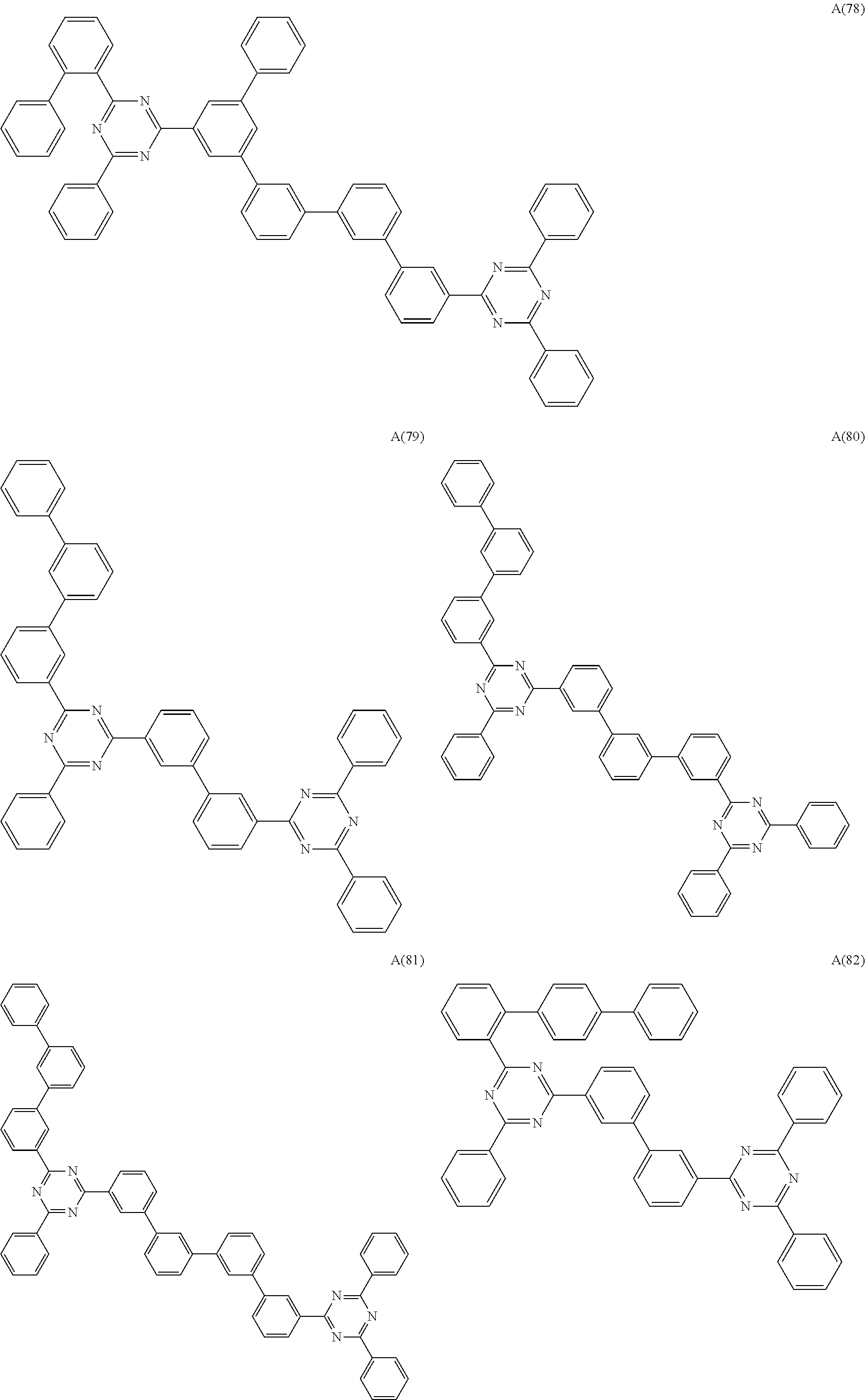

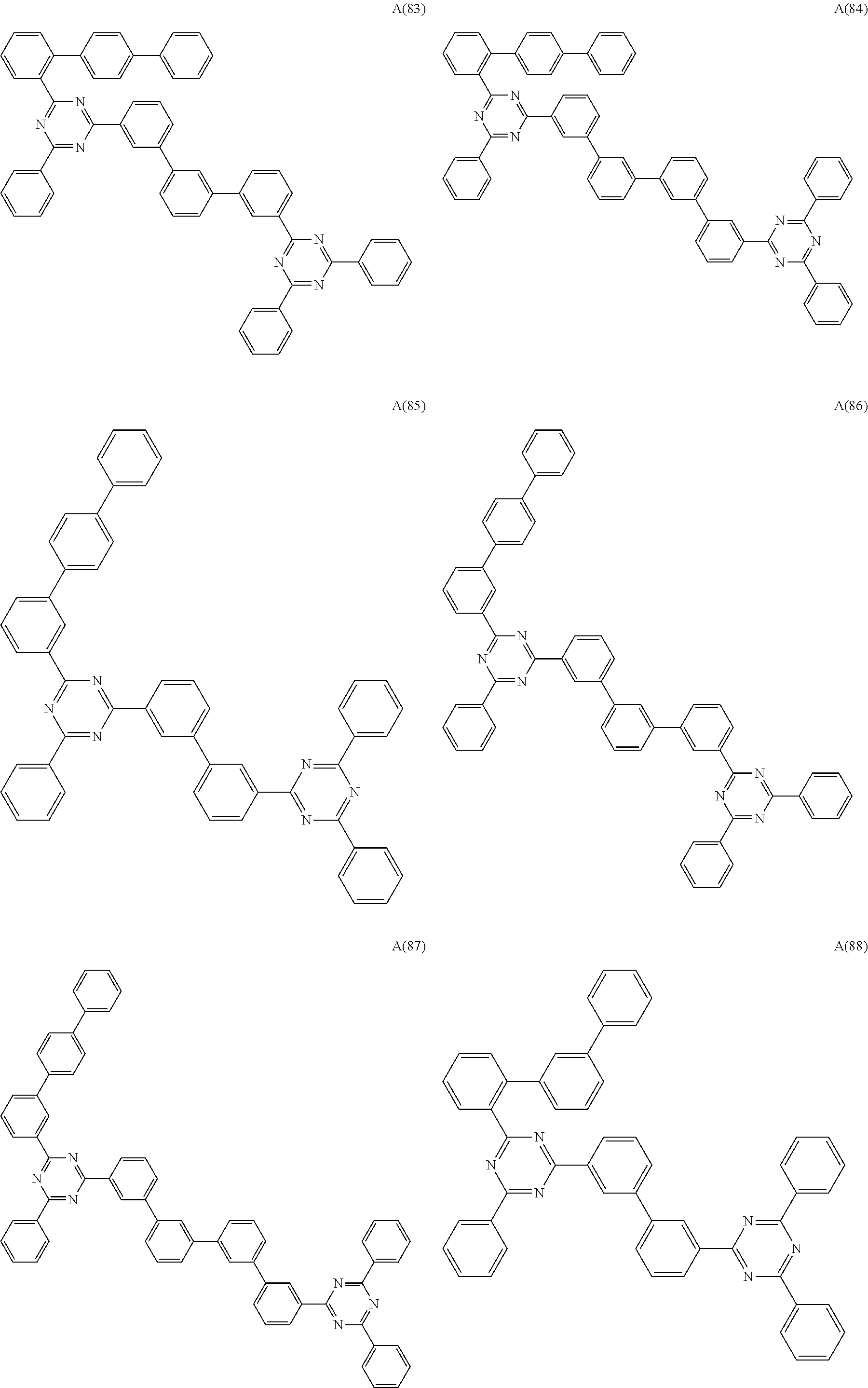

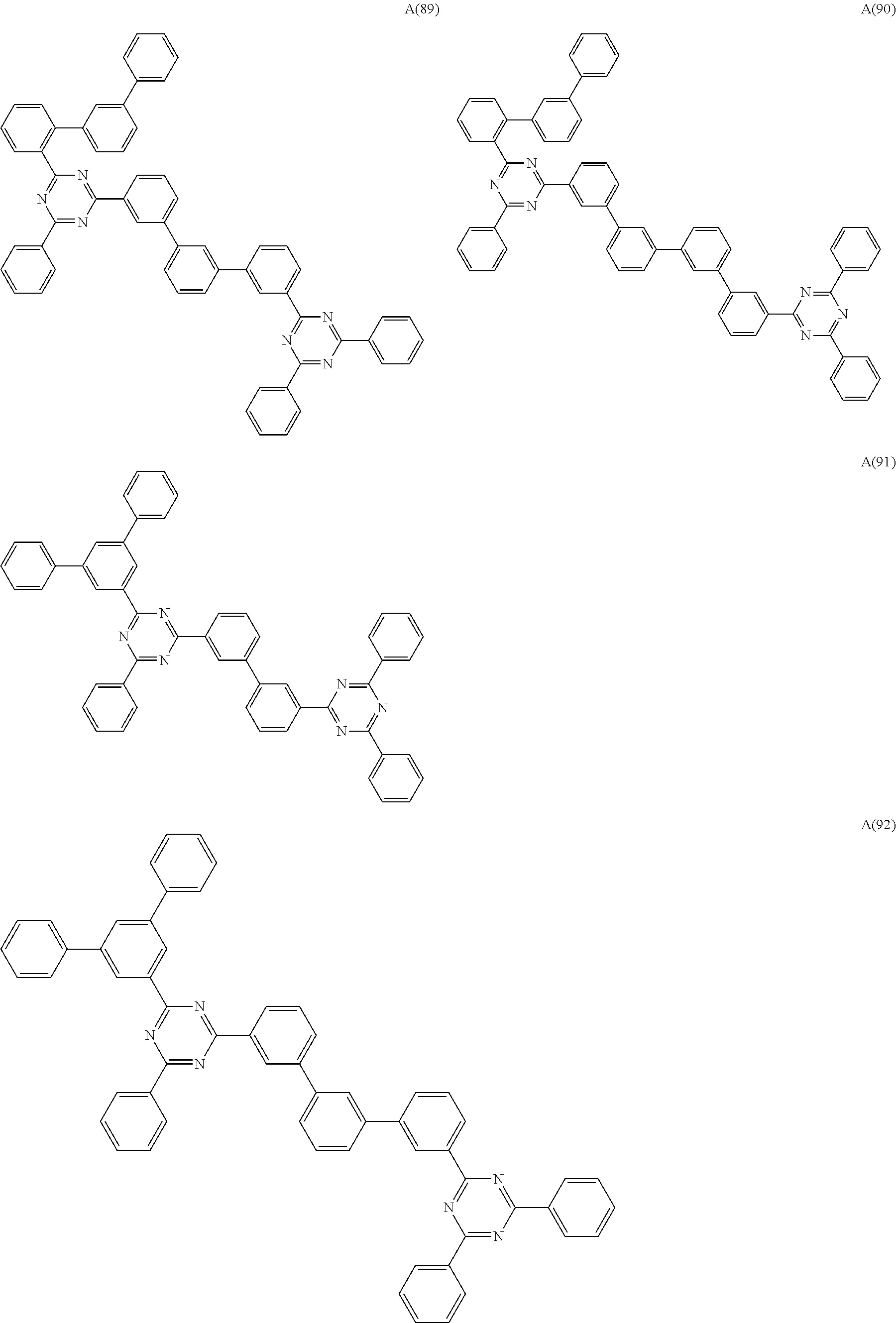 [SMILES: c1ccc(-c2cccc(-c3ccccc3-c3nc(-c4ccccc4)nc(-c4cccc(-c5cccc(-c6cccc(-c7nc(-c8ccccc8)nc(-c8ccccc8)n7)c6)c5)c4)n3)c2)cc1 ; c1ccc(-c2cccc(-c3ccccc3-c3nc(-c4ccccc4)nc(-c4cccc(-c5cccc(-c6cccc(-c7cccc(-c8nc(-c9ccccc9)nc(-c9ccccc9)n8)c7)c6)c5)c4)n3)c2)cc1 ; c1ccc(-c2cc(-c3ccccc3)cc(-c3nc(-c4ccccc4)nc(-c4cccc(-c5cccc(-c6nc(-c7ccccc7)nc(-c7ccccc7)n6)c5)c4)n3)c2)cc1 ; c1ccc(-c2cc(-c3ccccc3)cc(-c3nc(-c4ccccc4)nc(-c4cccc(-c5cccc(-c6cccc(-c7nc(-c8ccccc8)nc(-c8ccccc8)n7)c6)c5)c4)n3)c2)cc1]

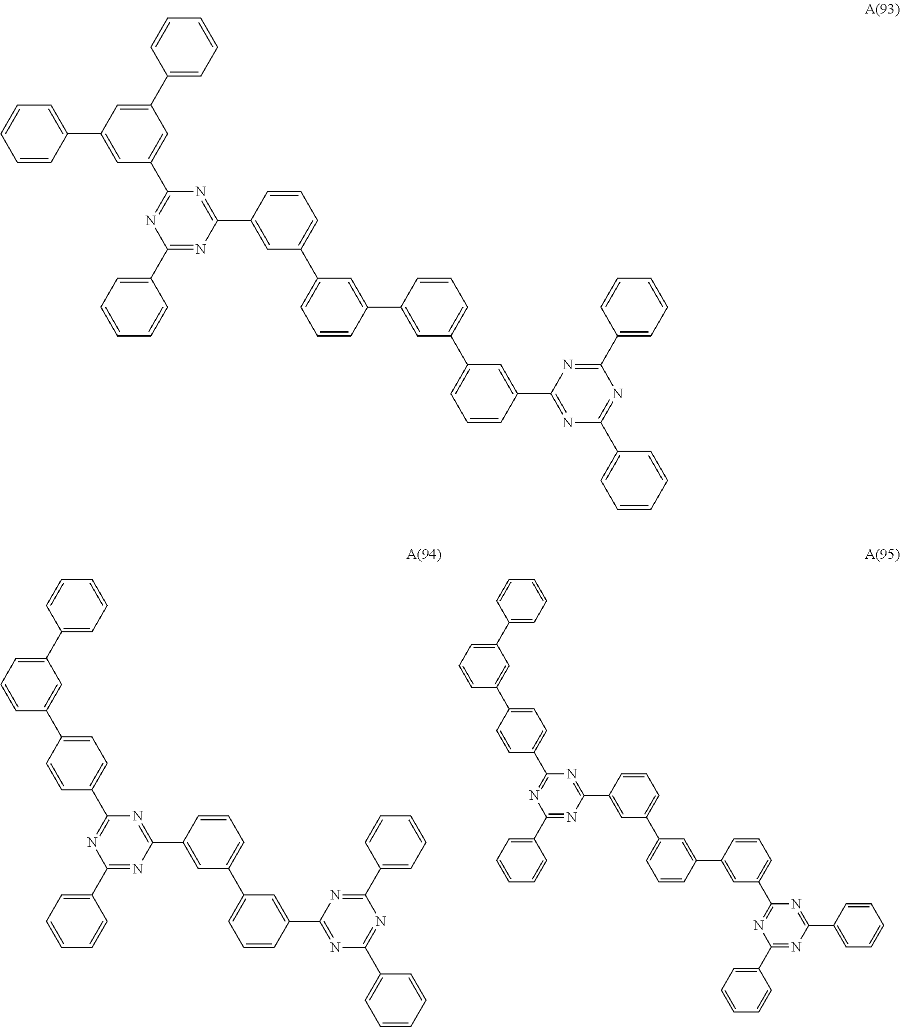 [SMILES: c1ccc(-c2cc(-c3ccccc3)cc(-c3nc(-c4ccccc4)nc(-c4cccc(-c5cccc(-c6cccc(-c7cccc(-c8nc(-c9ccccc9)nc(-c9ccccc9)n8)c7)c6)c5)c4)n3)c2)cc1 ; c1ccc(-c2cccc(-c3ccc(-c4nc(-c5ccccc5)nc(-c5cccc(-c6cccc(-c7nc(-c8ccccc8)nc(-c8ccccc8)n7)c6)c5)n4)cc3)c2)cc1 ; c1ccc(-c2cccc(-c3ccc(-c4nc(-c5ccccc5)nc(-c5cccc(-c6cccc(-c7cccc(-c8nc(-c9ccccc9)nc(-c9ccccc9)n8)c7)c6)c5)n4)cc3)c2)cc1]

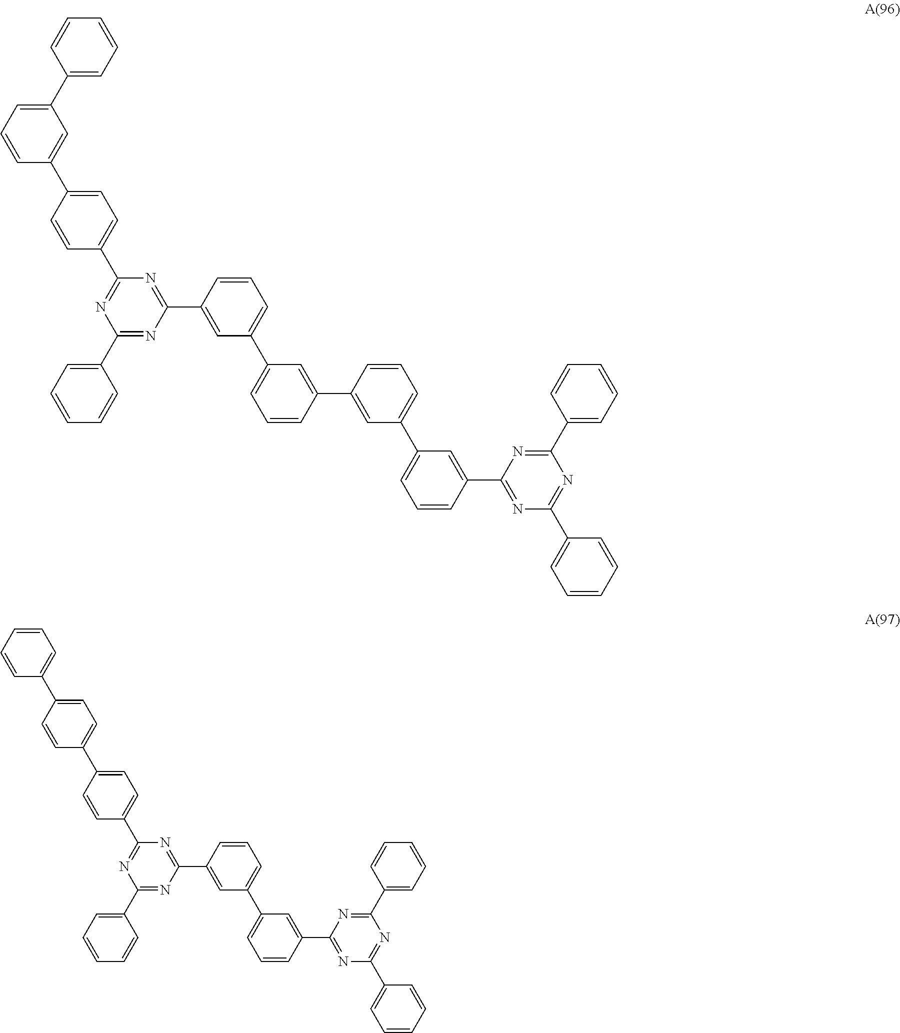 [SMILES: c1ccc(-c2cccc(-c3ccc(-c4nc(-c5ccccc5)nc(-c5cccc(-c6cccc(-c7cccc(-c8cccc(-c9nc(-c%10ccccc%10)nc(-c%10ccccc%10)n9)c8)c7)c6)c5)n4)cc3)c2)cc1 ; c1ccc(-c2ccc(-c3ccc(-c4nc(-c5ccccc5)nc(-c5cccc(-c6cccc(-c7nc(-c8ccccc8)nc(-c8ccccc8)n7)c6)c5)n4)cc3)cc2)cc1]

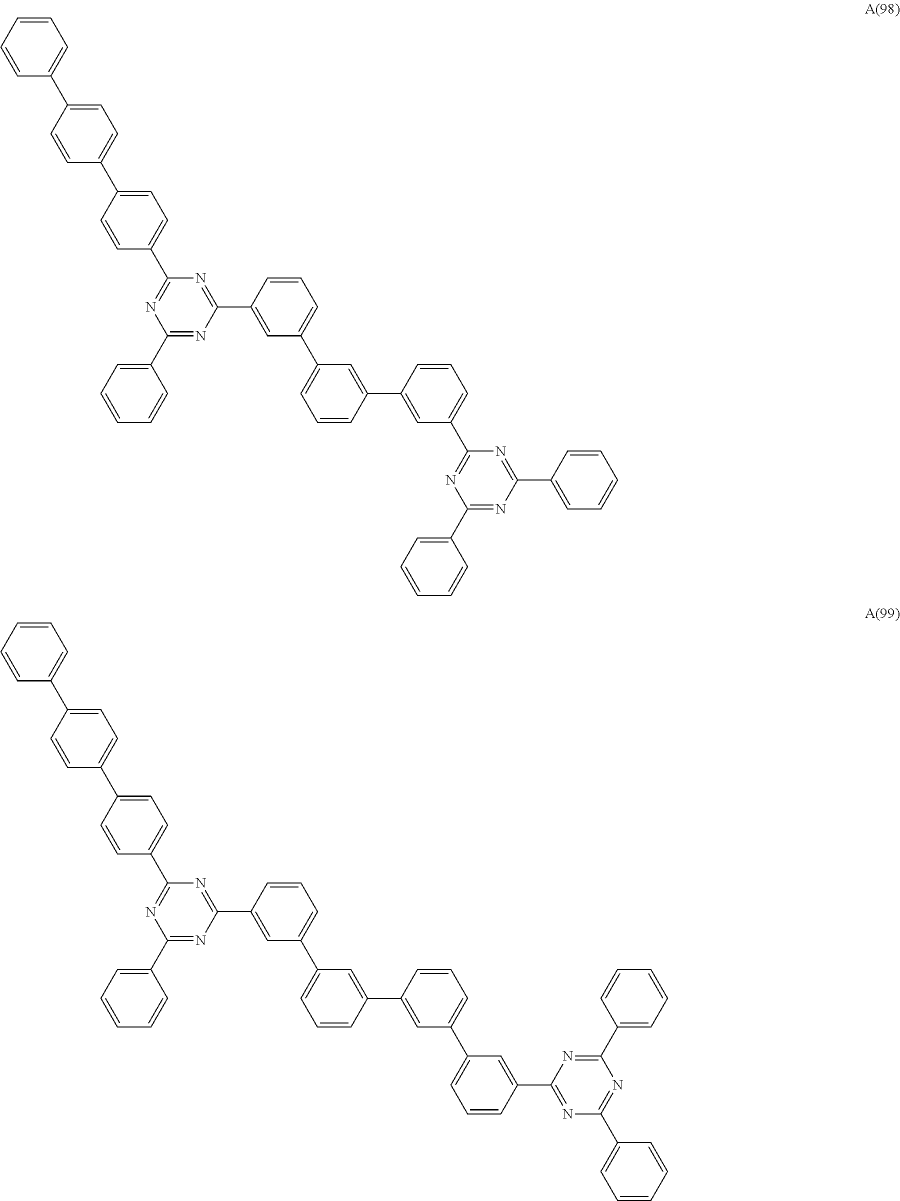 [SMILES: c1ccc(-c2ccc(-c3ccc(-c4nc(-c5ccccc5)nc(-c5cccc(-c6cccc(-c7cccc(-c8nc(-c9ccccc9)nc(-c9ccccc9)n8)c7)c6)c5)n4)cc3)cc2)cc1 ; c1ccc(-c2ccc(-c3ccc(-c4nc(-c5ccccc5)nc(-c5cccc(-c6cccc(-c7cccc(-c8cccc(-c9nc(-c%10ccccc%10)nc(-c%10ccccc%10)n9)c8)c7)c6)c5)n4)cc3)cc2)cc1]

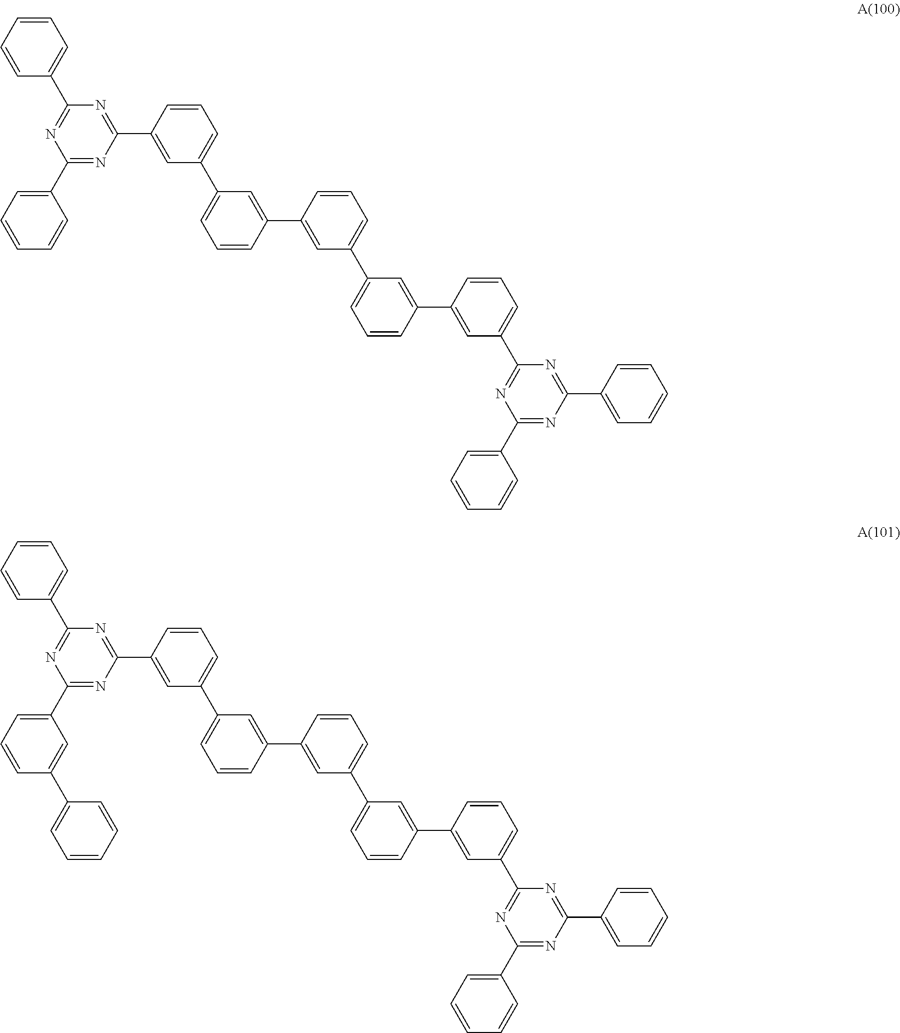 [SMILES: c1ccc(-c2nc(-c3ccccc3)nc(-c3cccc(-c4cccc(-c5cccc(-c6cccc(-c7cccc(-c8nc(-c9ccccc9)nc(-c9ccccc9)n8)c7)c6)c5)c4)c3)n2)cc1 ; c1ccc(-c2cccc(-c3nc(-c4ccccc4)nc(-c4cccc(-c5cccc(-c6cccc(-c7cccc(-c8cccc(-c9nc(-c%10ccccc%10)nc(-c%10ccccc%10)n9)c8)c7)c6)c5)c4)n3)c2)cc1]

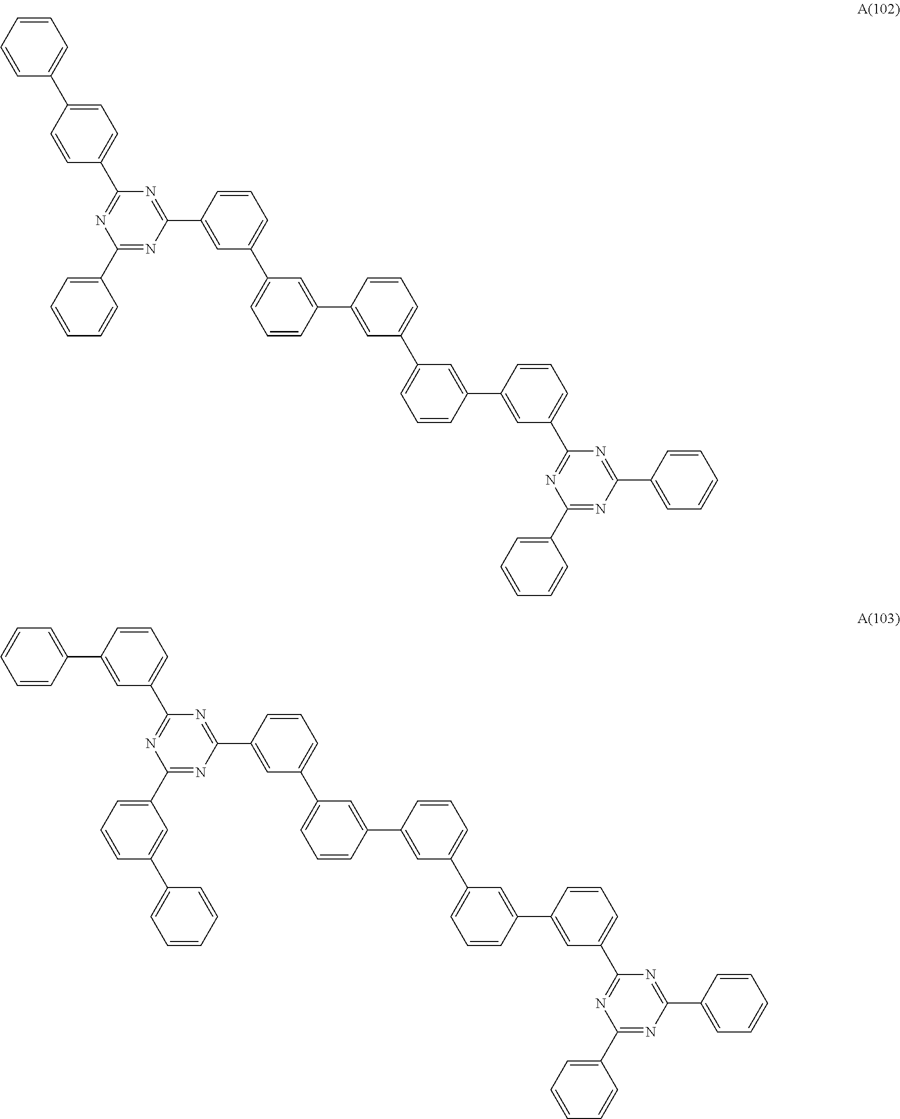 [SMILES: c1ccc(-c2ccc(-c3nc(-c4ccccc4)nc(-c4cccc(-c5cccc(-c6cccc(-c7cccc(-c8cccc(-c9nc(-c%10ccccc%10)nc(-c%10ccccc%10)n9)c8)c7)c6)c5)c4)n3)cc2)cc1 ; c1ccc(-c2cccc(-c3nc(-c4cccc(-c5ccccc5)c4)nc(-c4cccc(-c5cccc(-c6cccc(-c7cccc(-c8cccc(-c9nc(-c%10ccccc%10)nc(-c%10ccccc%10)n9)c8)c7)c6)c5)c4)n3)c2)cc1]

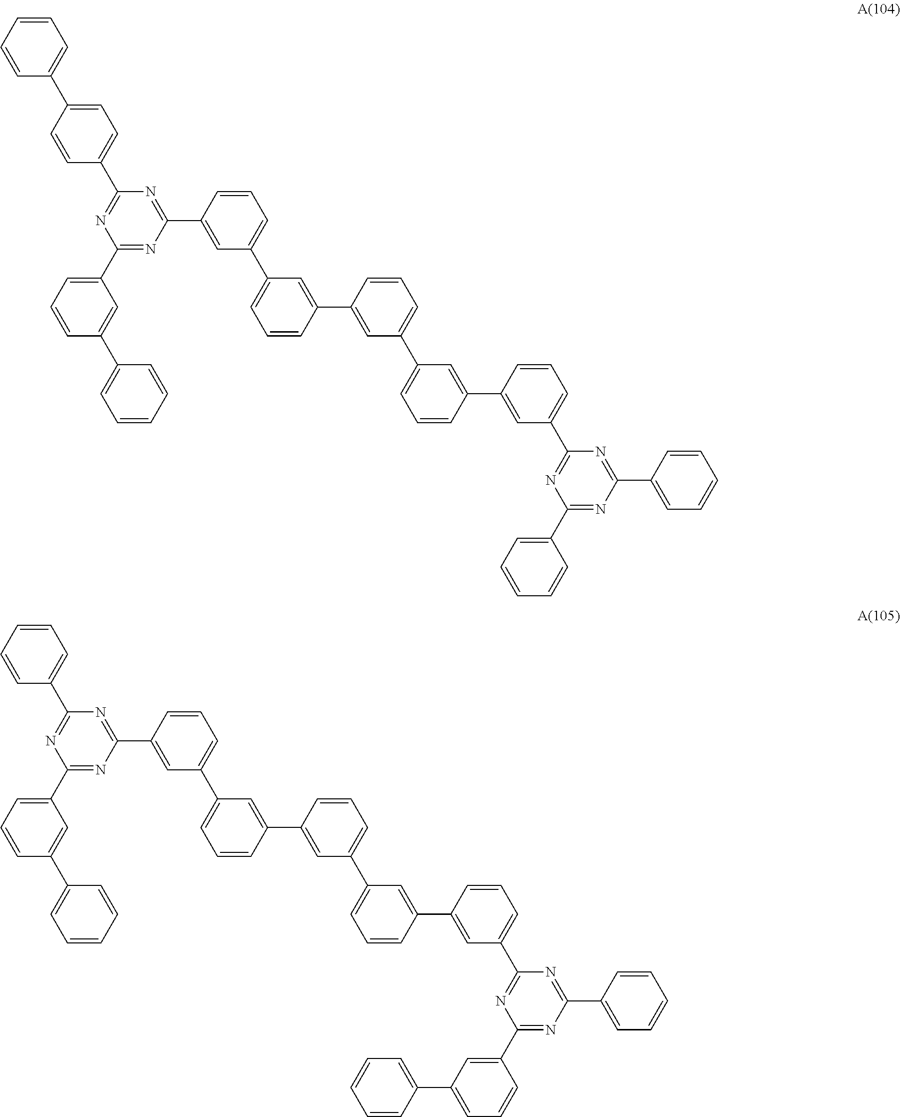 [SMILES: c1ccc(-c2ccc(-c3nc(-c4cccc(-c5ccccc5)c4)nc(-c4cccc(-c5cccc(-c6cccc(-c7cccc(-c8cccc(-c9nc(-c%10ccccc%10)nc(-c%10ccccc%10)n9)c8)c7)c6)c5)c4)n3)cc2)cc1 ; c1ccc(-c2cccc(-c3nc(-c4ccccc4)nc(-c4cccc(-c5cccc(-c6cccc(-c7cccc(-c8cccc(-c9nc(-c%10ccccc%10)nc(-c%10cccc(-c%11ccccc%11)c%10)n9)c8)c7)c6)c5)c4)n3)c2)cc1]

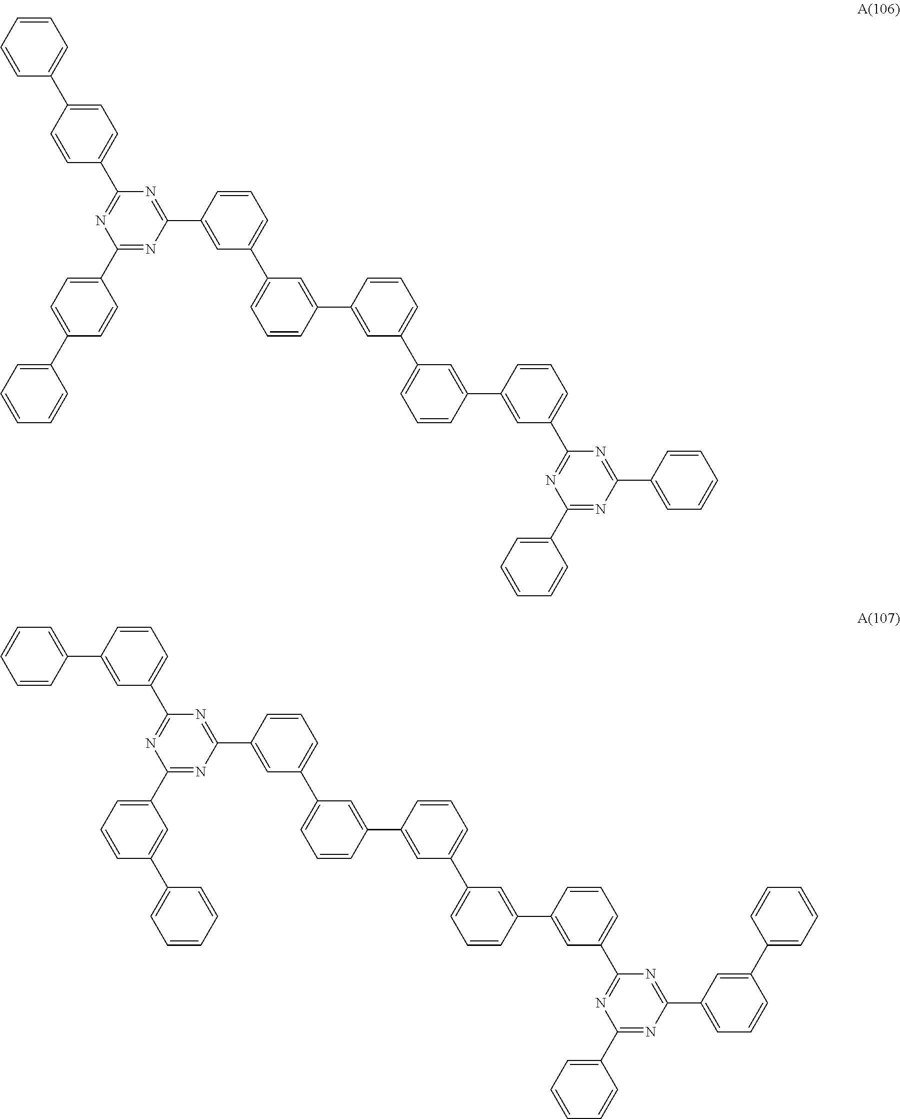 [SMILES: c1ccc(-c2ccc(-c3nc(-c4ccc(-c5ccccc5)cc4)nc(-c4cccc(-c5cccc(-c6cccc(-c7cccc(-c8cccc(-c9nc(-c%10ccccc%10)nc(-c%10ccccc%10)n9)c8)c7)c6)c5)c4)n3)cc2)cc1 ; c1ccc(-c2cccc(-c3nc(-c4ccccc4)nc(-c4cccc(-c5cccc(-c6cccc(-c7cccc(-c8cccc(-c9nc(-c%10cccc(-c%11ccccc%11)c%10)nc(-c%10cccc(-c%11ccccc%11)c%10)n9)c8)c7)c6)c5)c4)n3)c2)cc1]

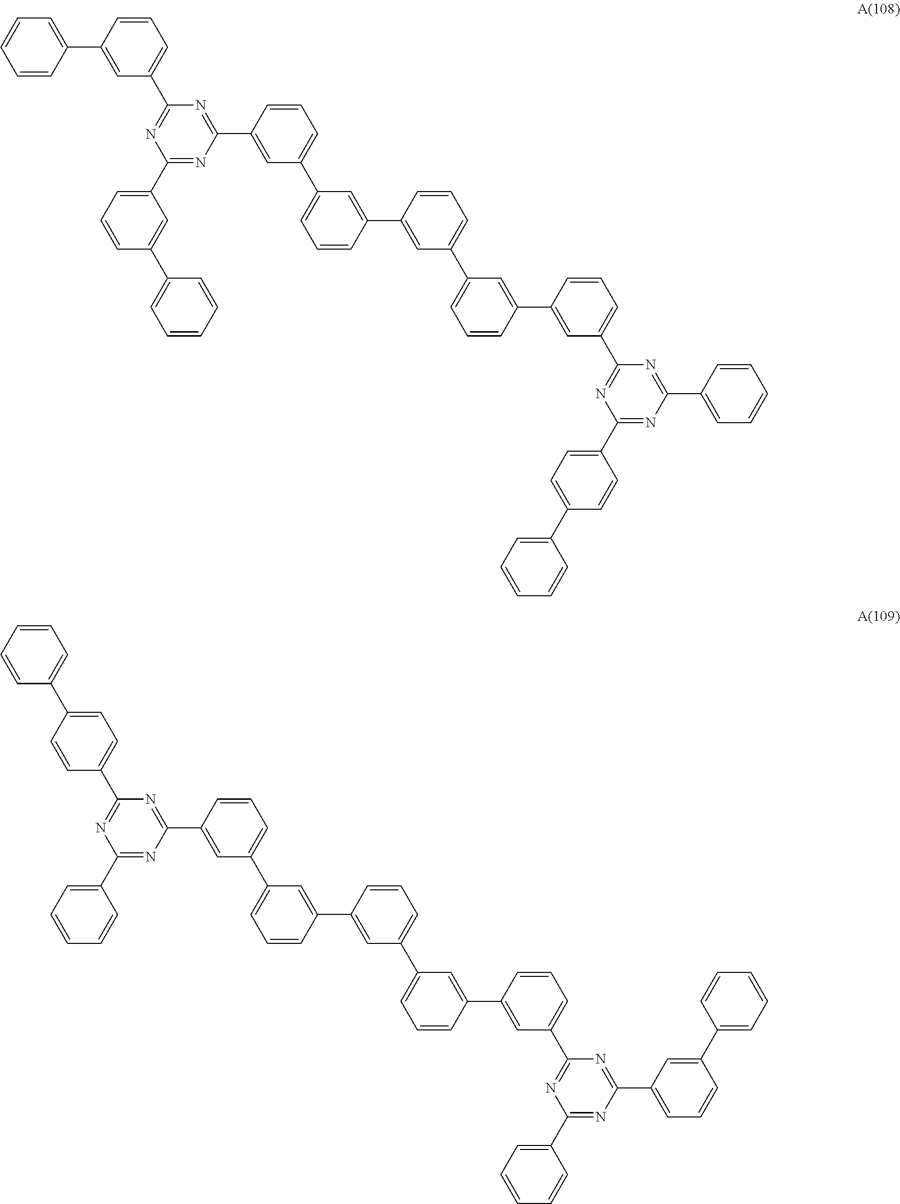 [SMILES: c1ccc(-c2ccc(-c3nc(-c4ccccc4)nc(-c4cccc(-c5cccc(-c6cccc(-c7cccc(-c8cccc(-c9nc(-c%10cccc(-c%11ccccc%11)c%10)nc(-c%10cccc(-c%11ccccc%11)c%10)n9)c8)c7)c6)c5)c4)n3)cc2)cc1 ; c1ccc(-c2ccc(-c3nc(-c4ccccc4)nc(-c4cccc(-c5cccc(-c6cccc(-c7cccc(-c8cccc(-c9nc(-c%10ccccc%10)nc(-c%10cccc(-c%11ccccc%11)c%10)n9)c8)c7)c6)c5)c4)n3)cc2)cc1]

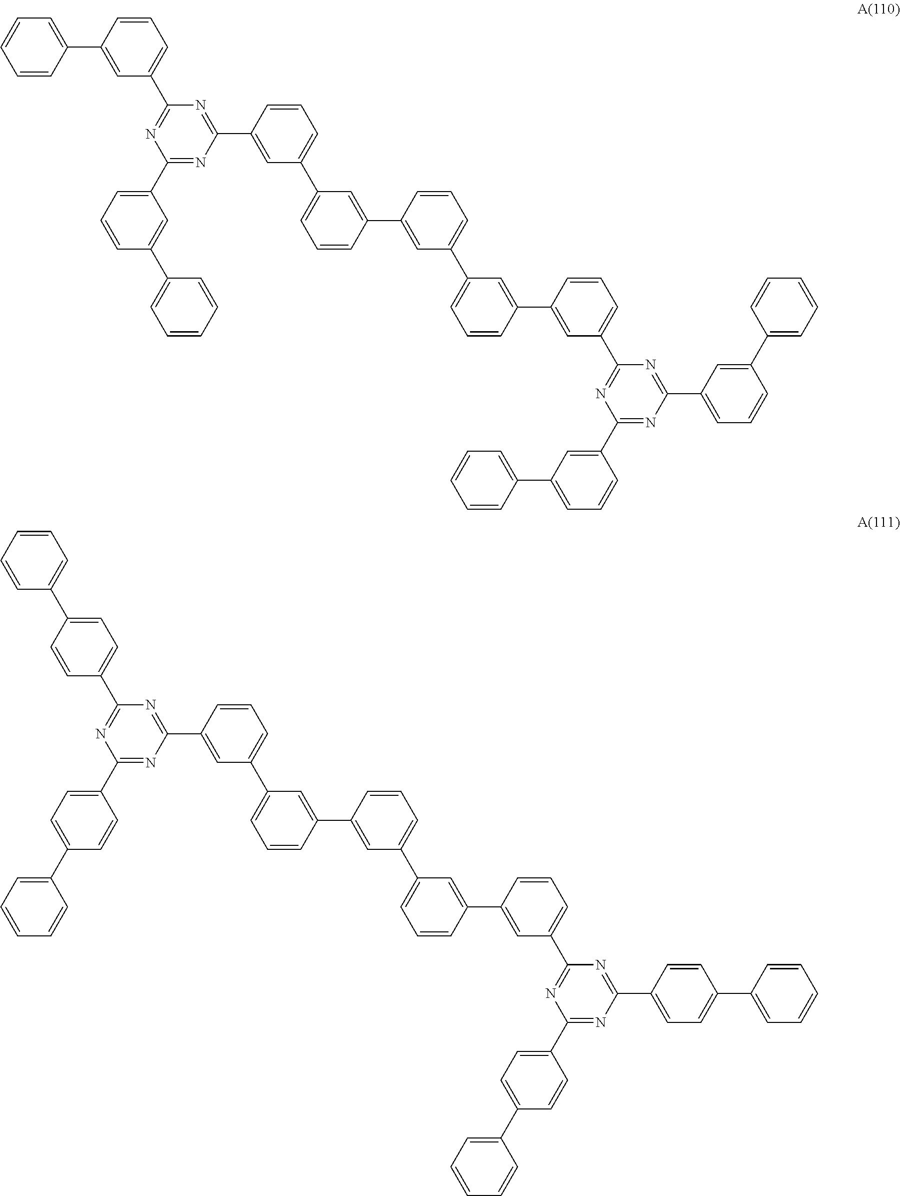 [SMILES: c1ccc(-c2cccc(-c3nc(-c4cccc(-c5ccccc5)c4)nc(-c4cccc(-c5cccc(-c6cccc(-c7cccc(-c8cccc(-c9nc(-c%10cccc(-c%11ccccc%11)c%10)nc(-c%10cccc(-c%11ccccc%11)c%10)n9)c8)c7)c6)c5)c4)n3)c2)cc1 ; c1ccc(-c2ccc(-c3nc(-c4ccc(-c5ccccc5)cc4)nc(-c4cccc(-c5cccc(-c6cccc(-c7cccc(-c8cccc(-c9nc(-c%10ccc(-c%11ccccc%11)cc%10)nc(-c%10ccc(-c%11ccccc%11)cc%10)n9)c8)c7)c6)c5)c4)n3)cc2)cc1]

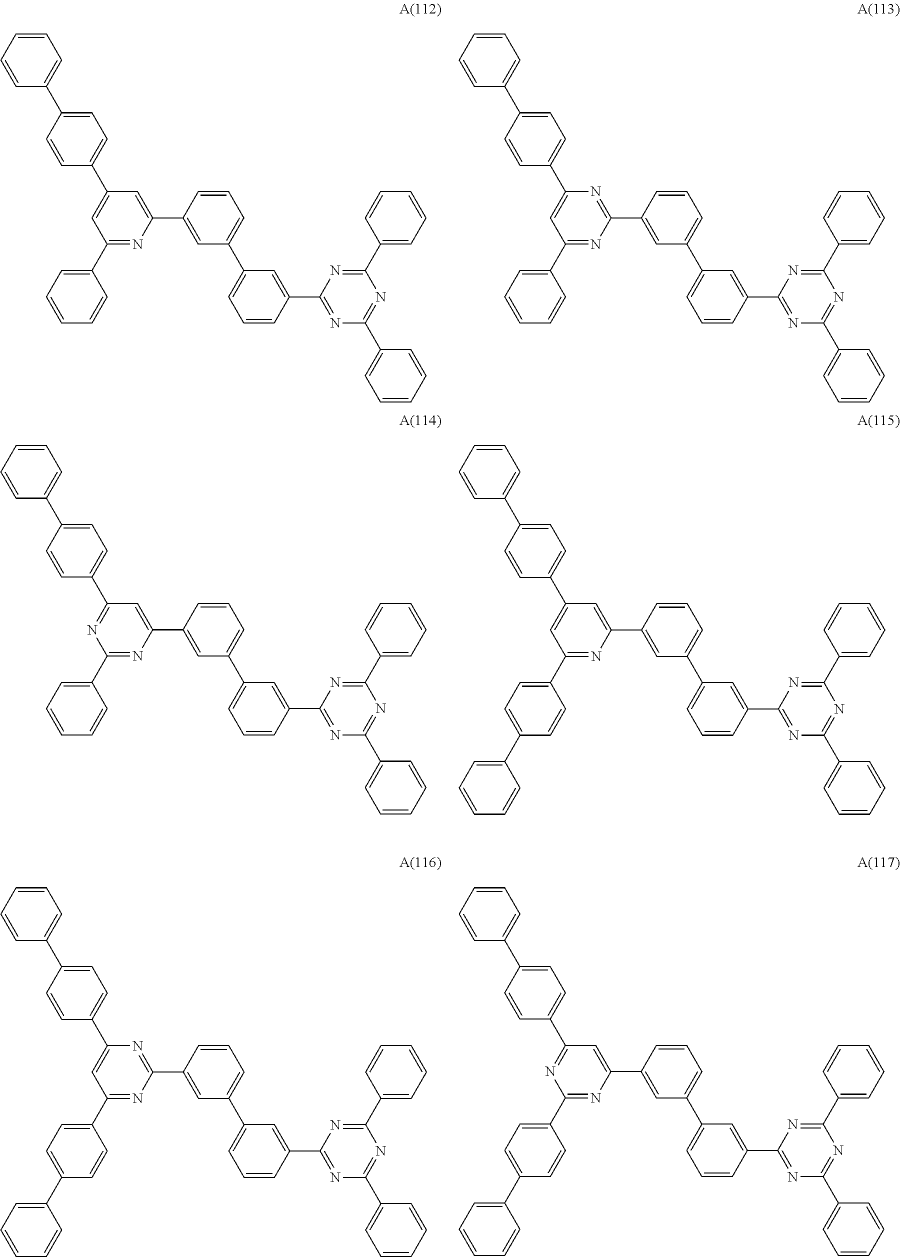

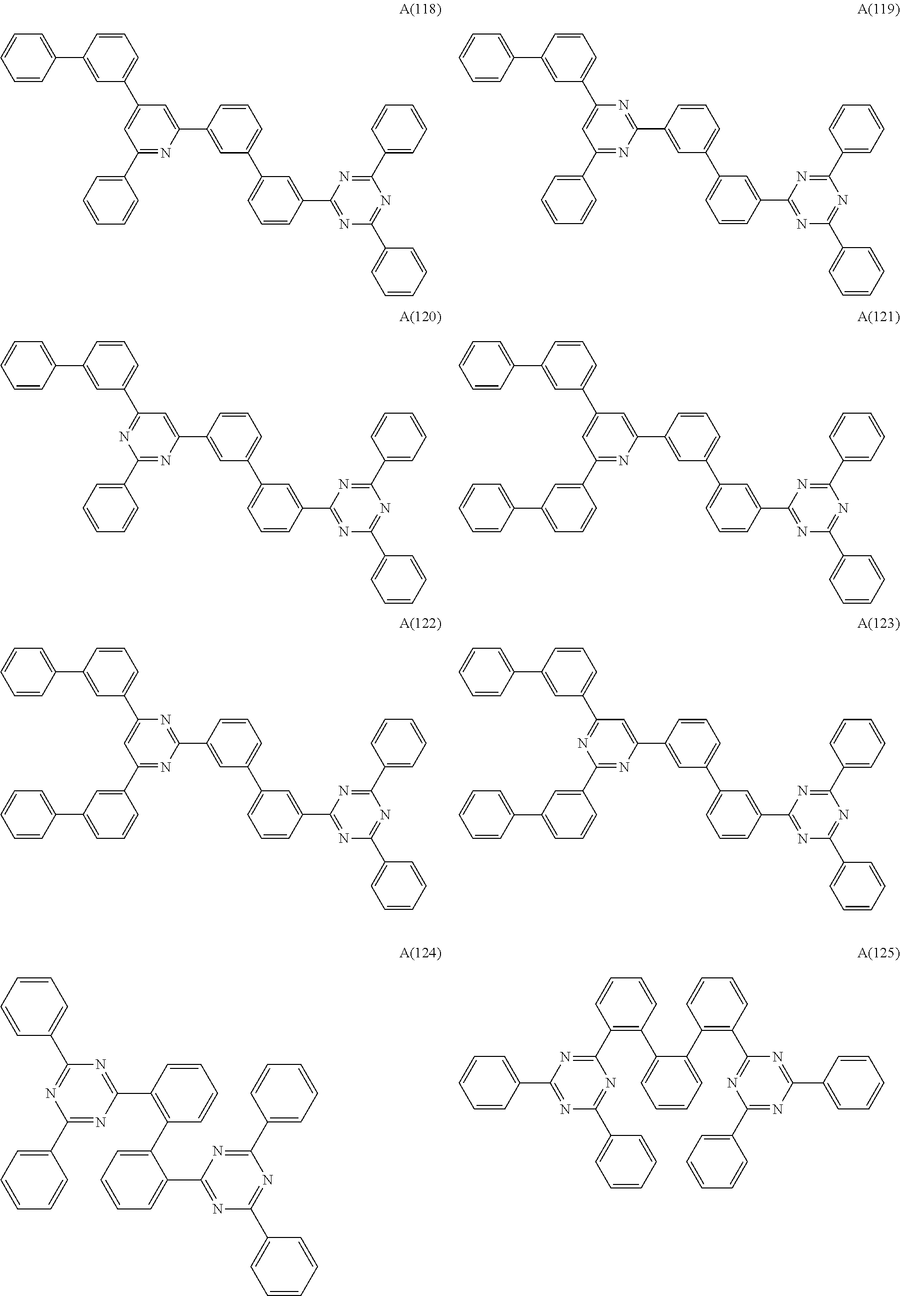

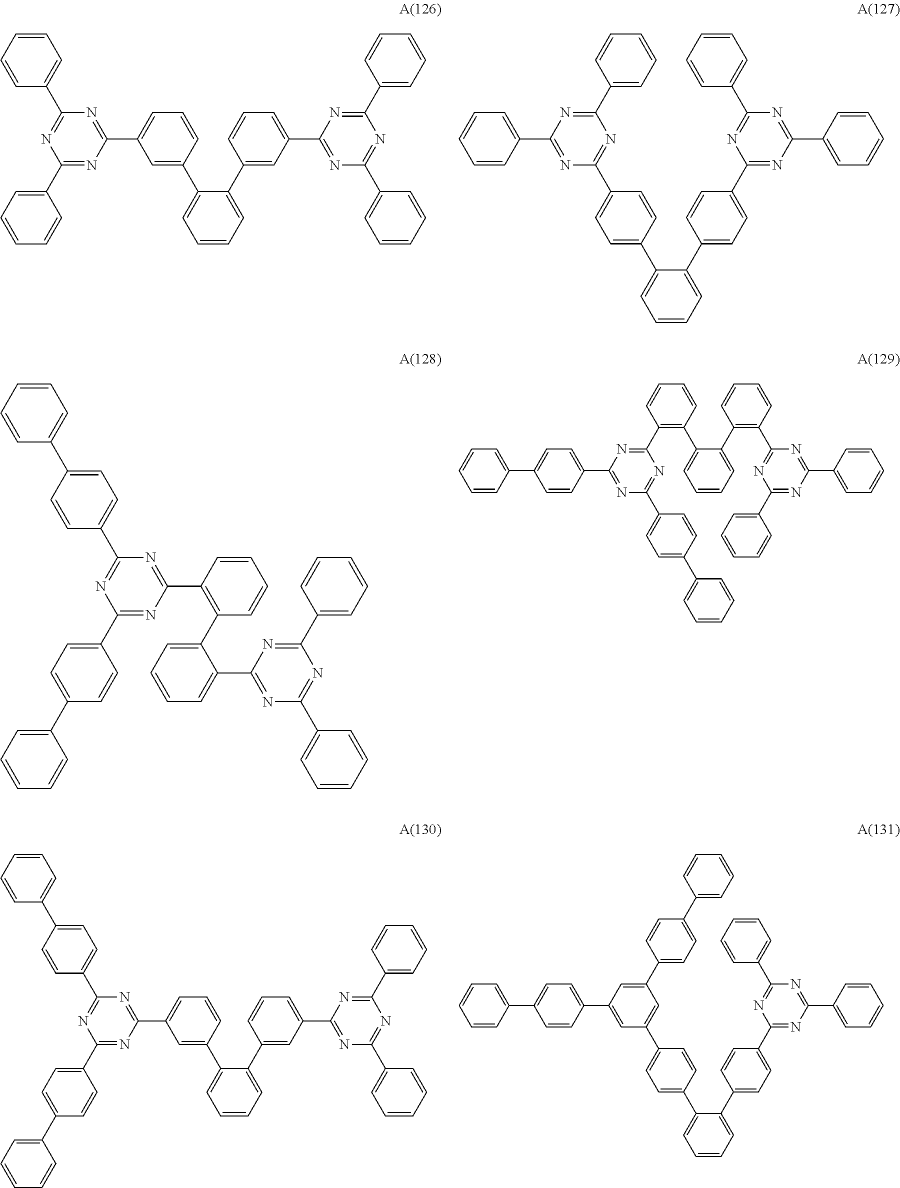

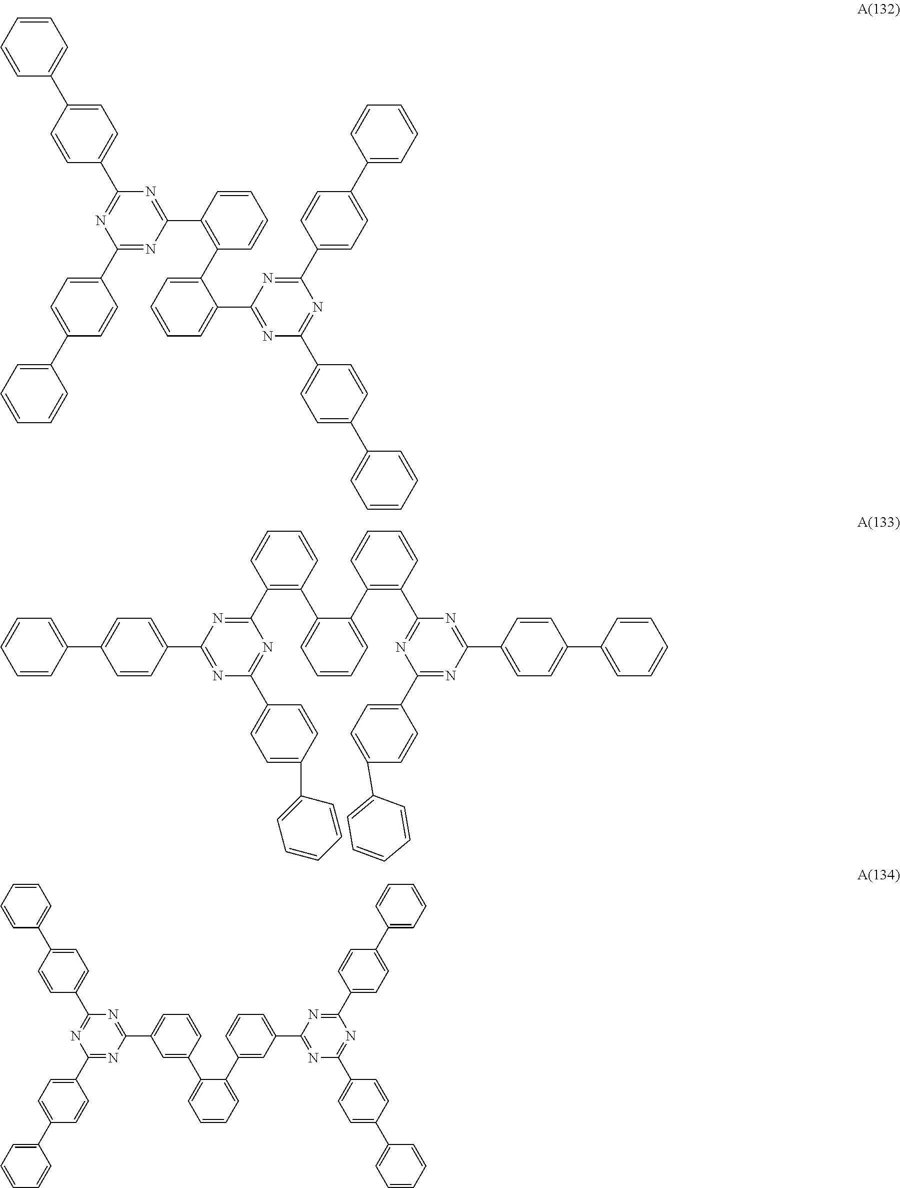

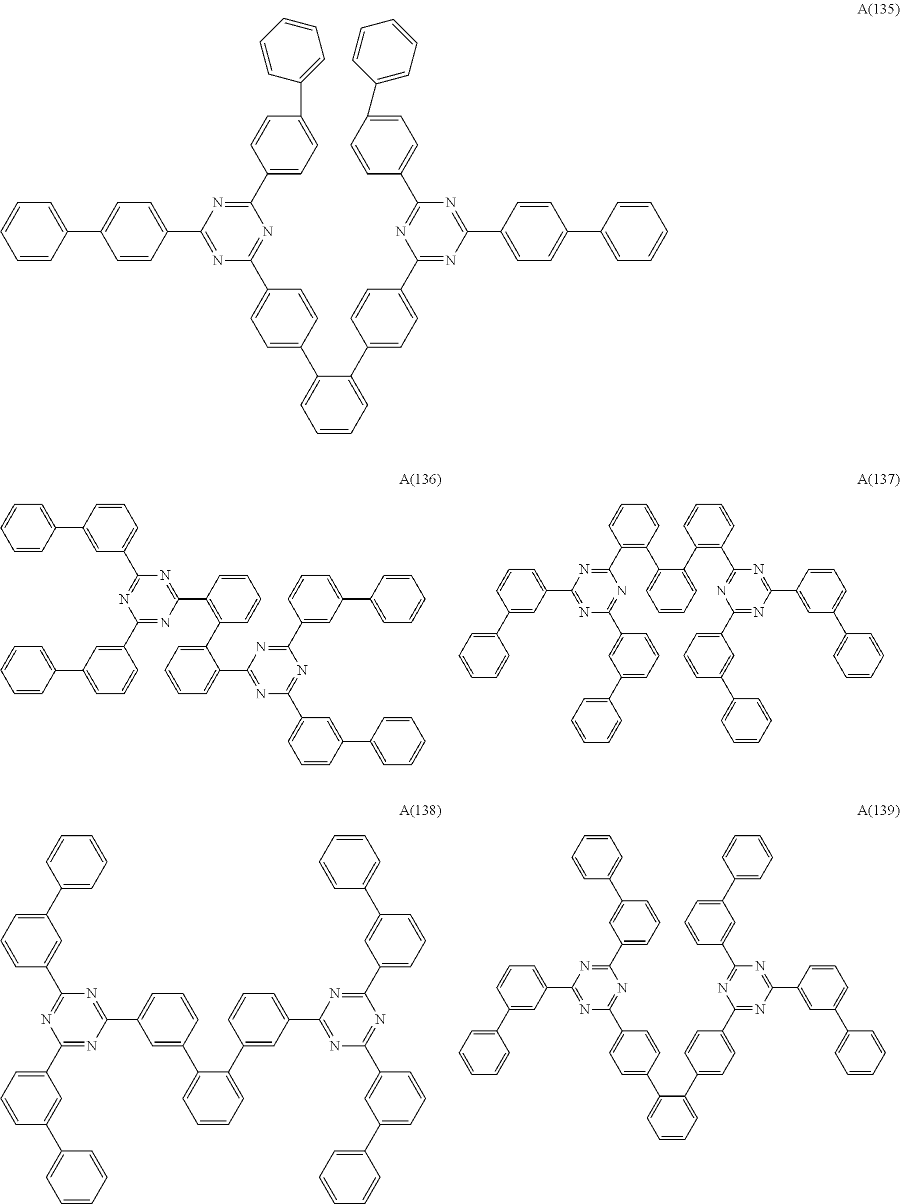

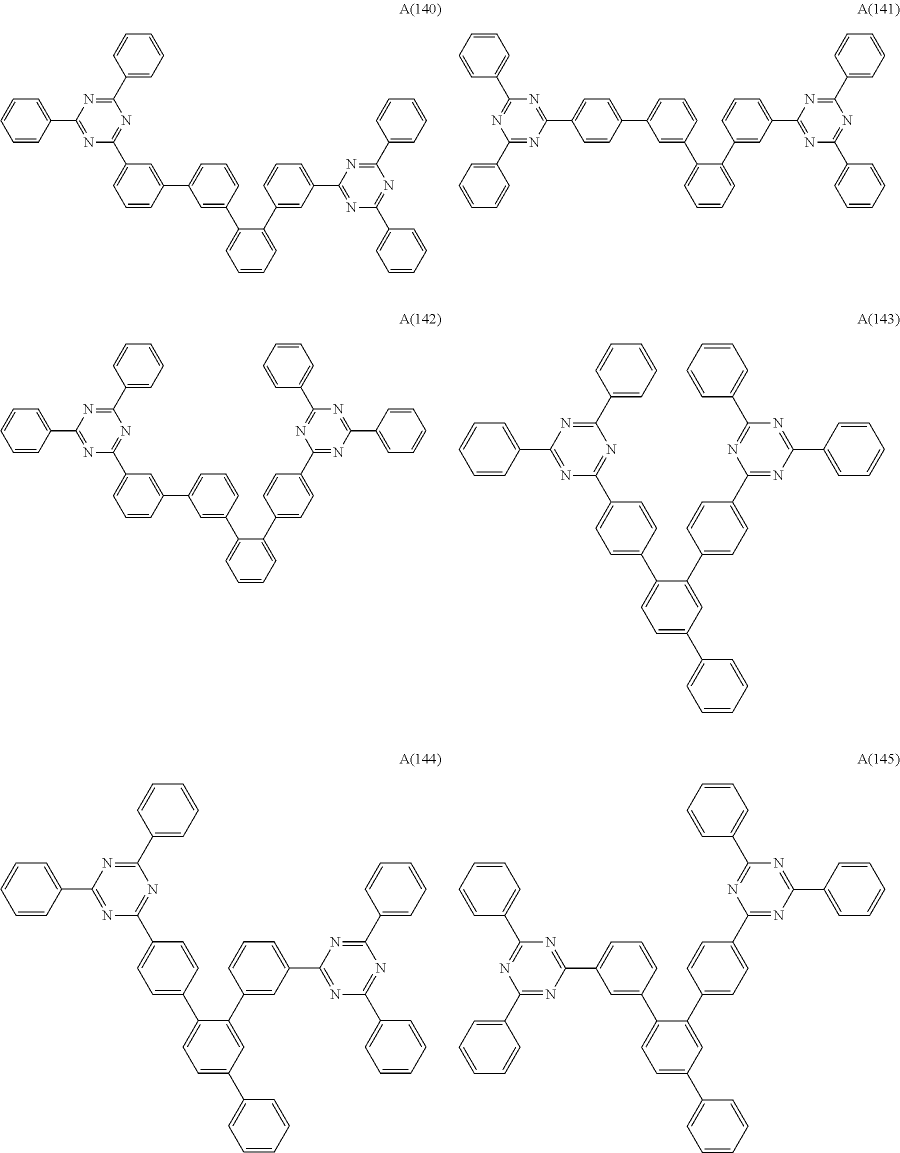

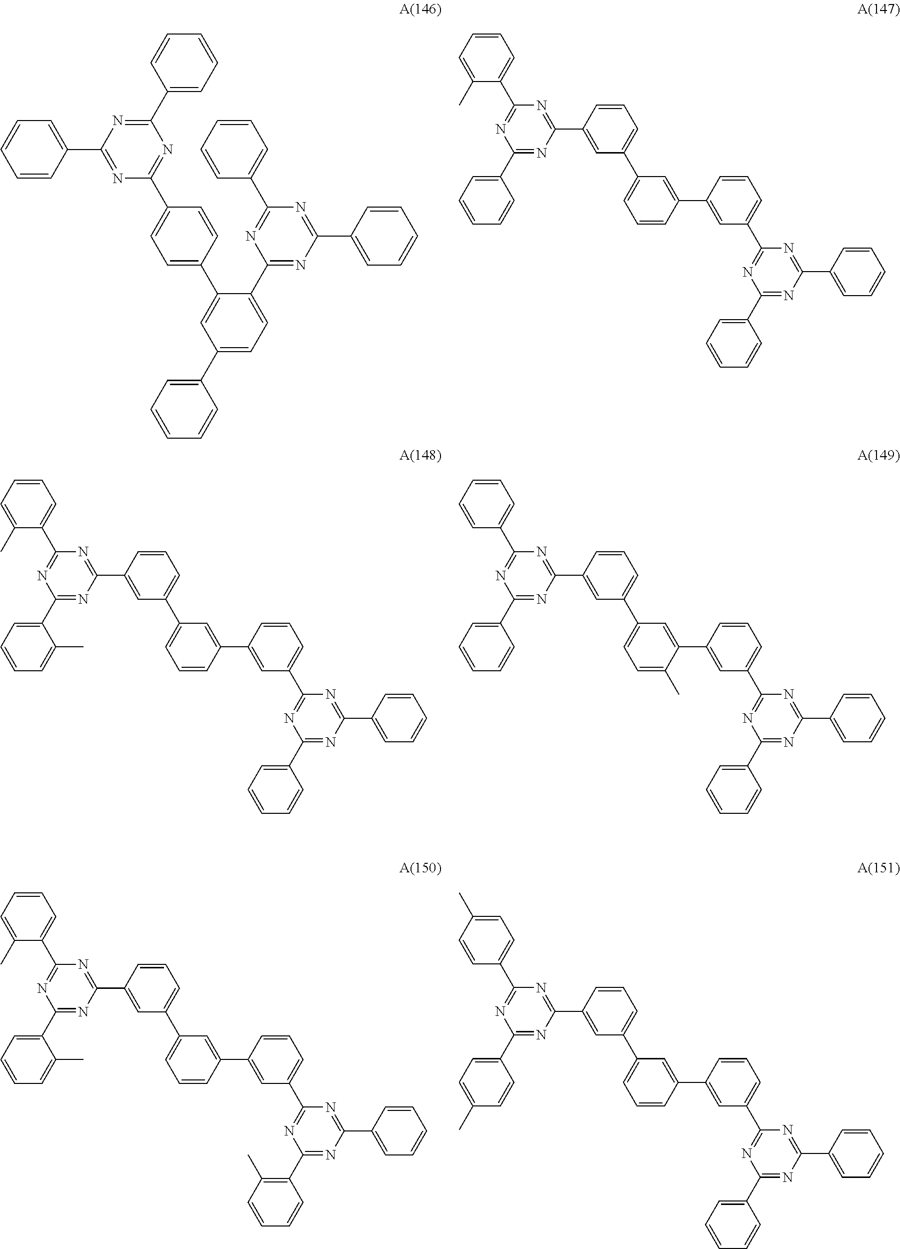

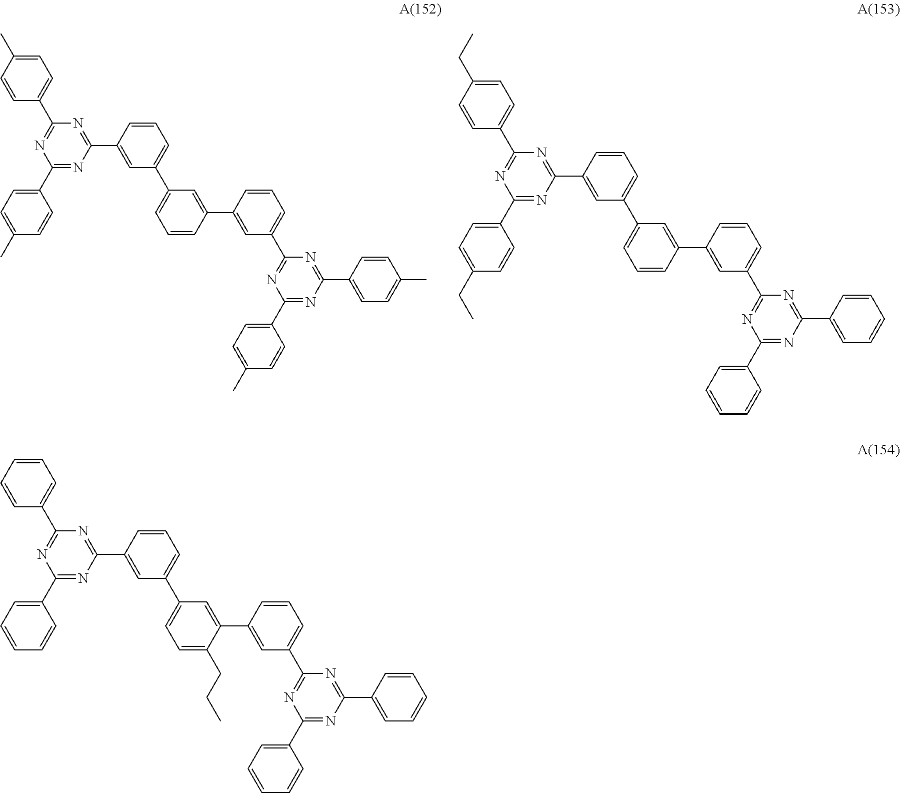

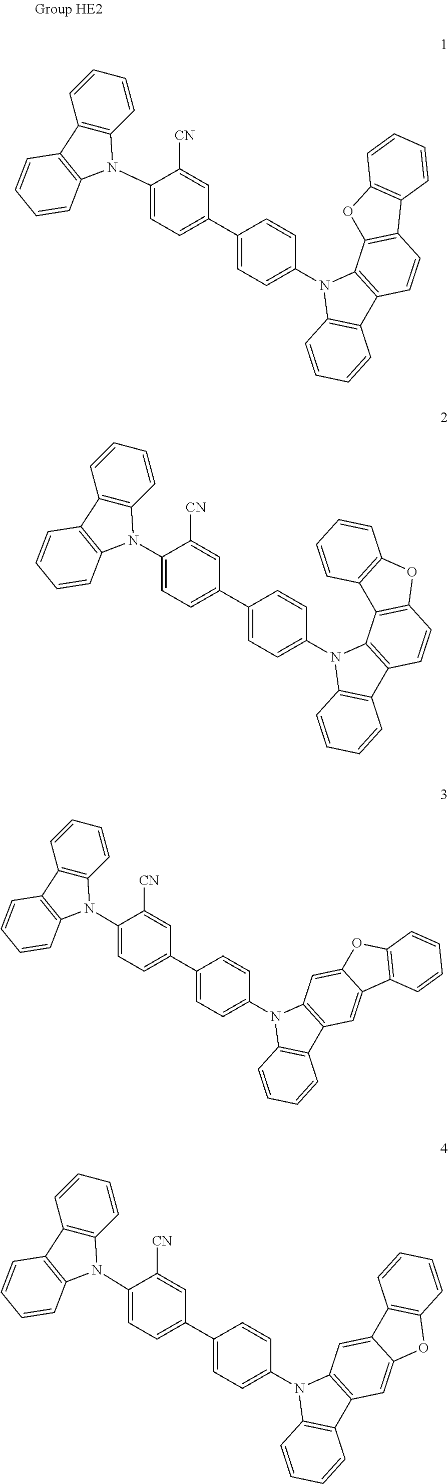 [SMILES: N#Cc1cc(-c2ccc(-n3c4ccccc4c4ccc5c6ccccc6oc5c43)cc2)ccc1-n1c2ccccc2c2ccccc21 ; N#Cc1cc(-c2ccc(-n3c4ccccc4c4ccc5oc6ccccc6c5c43)cc2)ccc1-n1c2ccccc2c2ccccc21 ; N#Cc1cc(-c2ccc(-n3c4ccccc4c4cc5c(cc43)oc3ccccc35)cc2)ccc1-n1c2ccccc2c2ccccc21 ; N#Cc1cc(-c2ccc(-n3c4ccccc4c4cc5oc6ccccc6c5cc43)cc2)ccc1-n1c2ccccc2c2ccccc21]

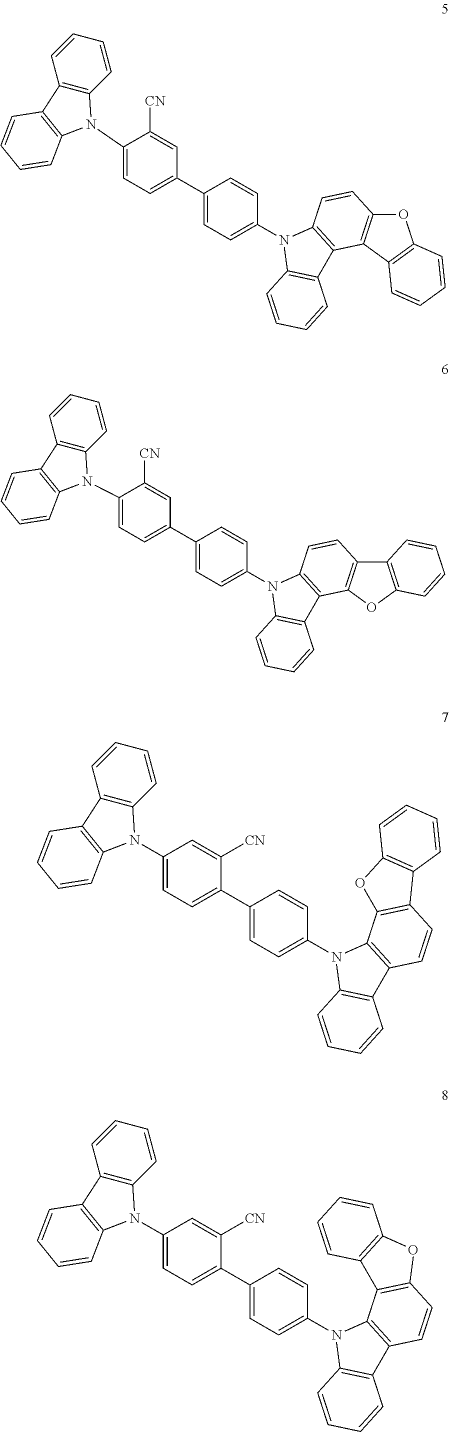 [SMILES: N#Cc1cc(-c2ccc(-n3c4ccccc4c4c5c(ccc43)oc3ccccc35)cc2)ccc1-n1c2ccccc2c2ccccc21 ; N#Cc1cc(-c2ccc(-n3c4ccccc4c4c5oc6ccccc6c5ccc43)cc2)ccc1-n1c2ccccc2c2ccccc21 ; N#Cc1cc(-n2c3ccccc3c3ccccc32)ccc1-c1ccc(-n2c3ccccc3c3ccc4c5ccccc5oc4c32)cc1 ; N#Cc1cc(-n2c3ccccc3c3ccccc32)ccc1-c1ccc(-n2c3ccccc3c3ccc4oc5ccccc5c4c32)cc1]

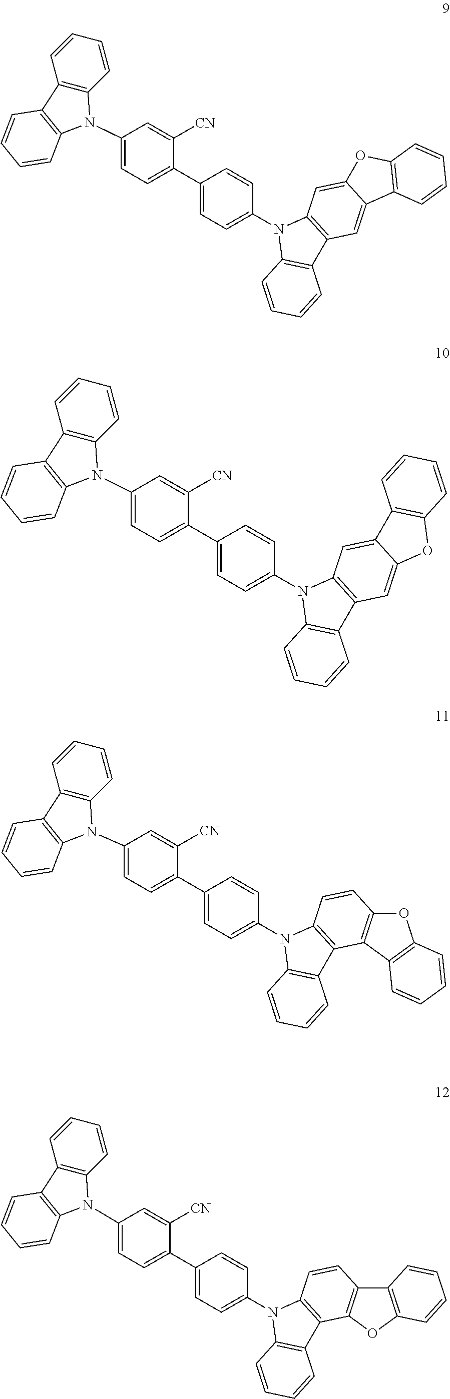 [SMILES: N#Cc1cc(-n2c3ccccc3c3ccccc32)ccc1-c1ccc(-n2c3ccccc3c3cc4c(cc32)oc2ccccc24)cc1 ; N#Cc1cc(-n2c3ccccc3c3ccccc32)ccc1-c1ccc(-n2c3ccccc3c3cc4oc5ccccc5c4cc32)cc1 ; N#Cc1cc(-n2c3ccccc3c3ccccc32)ccc1-c1ccc(-n2c3ccccc3c3c4c(ccc32)oc2ccccc24)cc1 ; N#Cc1cc(-n2c3ccccc3c3ccccc32)ccc1-c1ccc(-n2c3ccccc3c3c4oc5ccccc5c4ccc32)cc1]

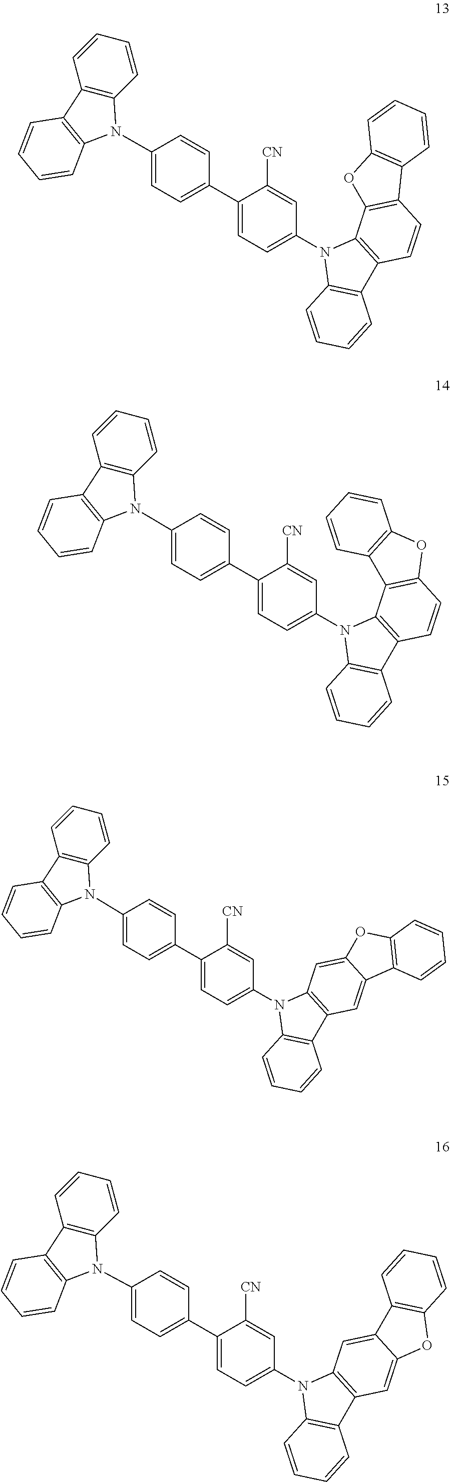 [SMILES: N#Cc1cc(-n2c3ccccc3c3ccc4c5ccccc5oc4c32)ccc1-c1ccc(-n2c3ccccc3c3ccccc32)cc1 ; N#Cc1cc(-n2c3ccccc3c3ccc4oc5ccccc5c4c32)ccc1-c1ccc(-n2c3ccccc3c3ccccc32)cc1 ; N#Cc1cc(-n2c3ccccc3c3cc4c(cc32)oc2ccccc24)ccc1-c1ccc(-n2c3ccccc3c3ccccc32)cc1 ; N#Cc1cc(-n2c3ccccc3c3cc4oc5ccccc5c4cc32)ccc1-c1ccc(-n2c3ccccc3c3ccccc32)cc1]

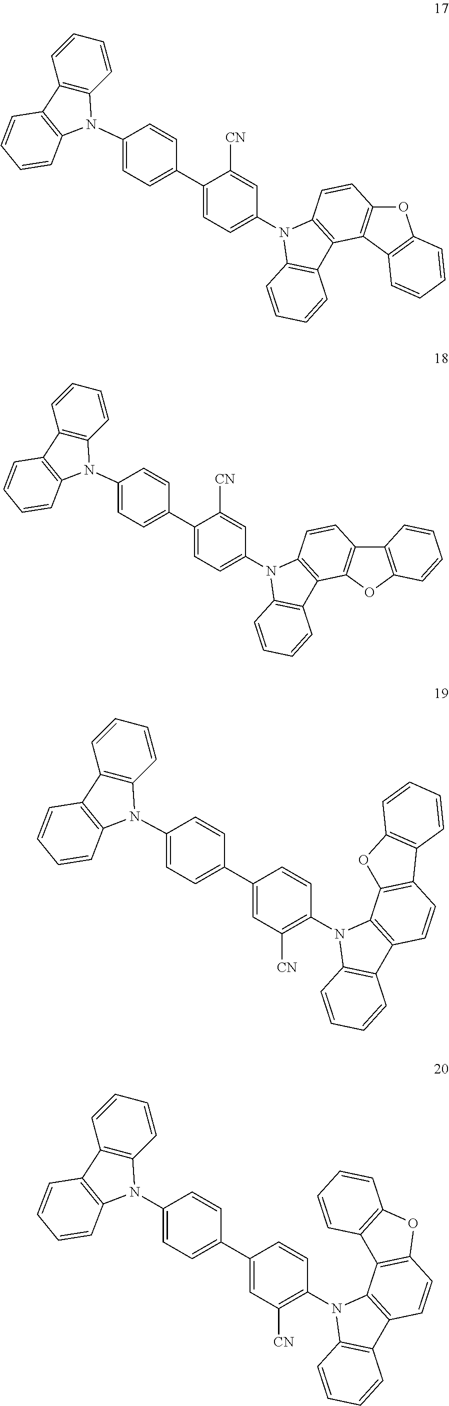 [SMILES: N#Cc1cc(-n2c3ccccc3c3c4c(ccc32)oc2ccccc24)ccc1-c1ccc(-n2c3ccccc3c3ccccc32)cc1 ; N#Cc1cc(-n2c3ccccc3c3c4oc5ccccc5c4ccc32)ccc1-c1ccc(-n2c3ccccc3c3ccccc32)cc1 ; N#Cc1cc(-c2ccc(-n3c4ccccc4c4ccccc43)cc2)ccc1-n1c2ccccc2c2ccc3c4ccccc4oc3c21 ; N#Cc1cc(-c2ccc(-n3c4ccccc4c4ccccc43)cc2)ccc1-n1c2ccccc2c2ccc3oc4ccccc4c3c21]

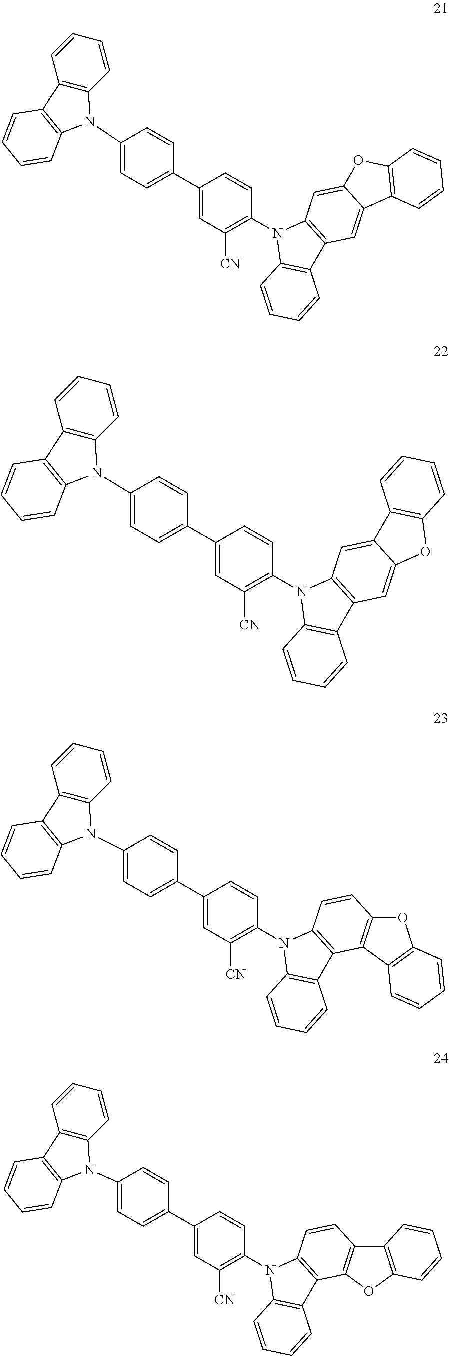 [SMILES: N#Cc1cc(-c2ccc(-n3c4ccccc4c4ccccc43)cc2)ccc1-n1c2ccccc2c2cc3c(cc21)oc1ccccc13 ; N#Cc1cc(-c2ccc(-n3c4ccccc4c4ccccc43)cc2)ccc1-n1c2ccccc2c2cc3oc4ccccc4c3cc21 ; N#Cc1cc(-c2ccc(-n3c4ccccc4c4ccccc43)cc2)ccc1-n1c2ccccc2c2c3c(ccc21)oc1ccccc13 ; N#Cc1cc(-c2ccc(-n3c4ccccc4c4ccccc43)cc2)ccc1-n1c2ccccc2c2c3oc4ccccc4c3ccc21]

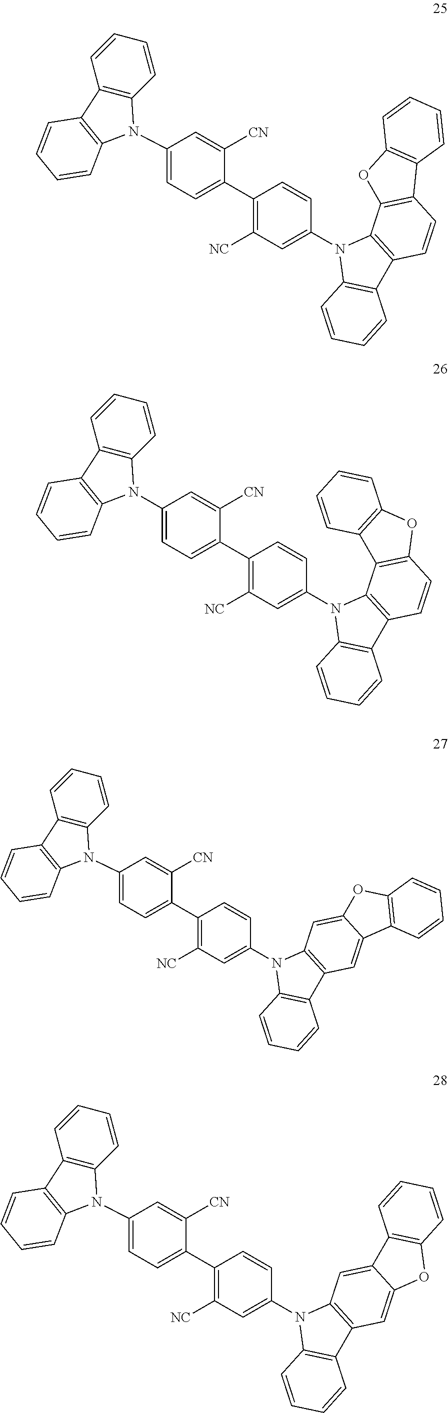 [SMILES: N#Cc1cc(-n2c3ccccc3c3ccccc32)ccc1-c1ccc(-n2c3ccccc3c3ccc4c5ccccc5oc4c32)cc1C#N ; N#Cc1cc(-n2c3ccccc3c3ccccc32)ccc1-c1ccc(-n2c3ccccc3c3ccc4oc5ccccc5c4c32)cc1C#N ; N#Cc1cc(-n2c3ccccc3c3ccccc32)ccc1-c1ccc(-n2c3ccccc3c3cc4c(cc32)oc2ccccc24)cc1C#N ; N#Cc1cc(-n2c3ccccc3c3ccccc32)ccc1-c1ccc(-n2c3ccccc3c3cc4oc5ccccc5c4cc32)cc1C#N]

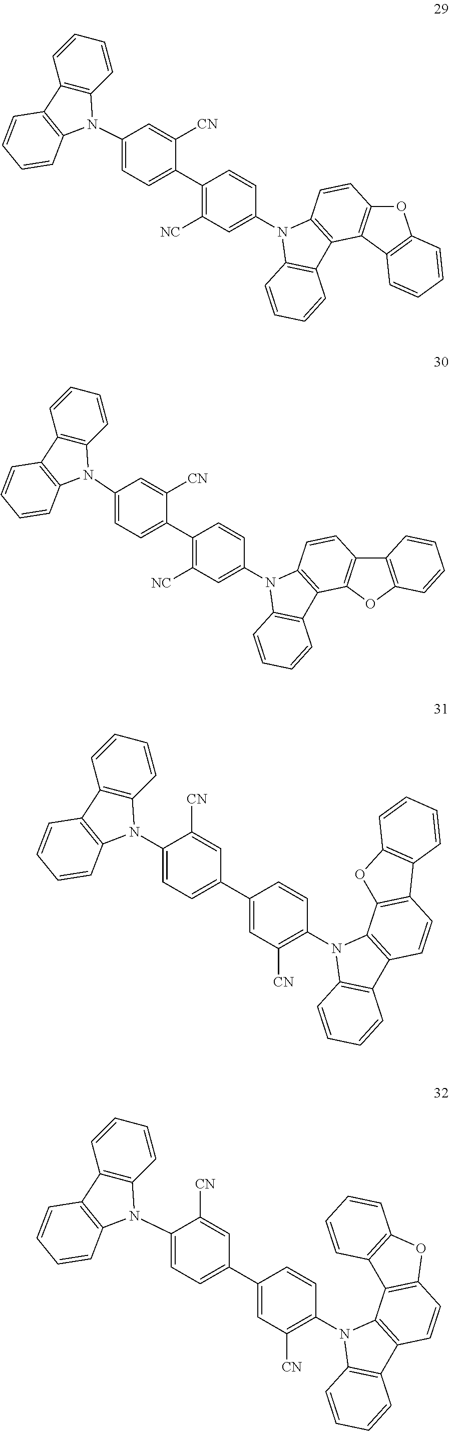 [SMILES: N#Cc1cc(-n2c3ccccc3c3ccccc32)ccc1-c1ccc(-n2c3ccccc3c3c4c(ccc32)oc2ccccc24)cc1C#N ; N#Cc1cc(-n2c3ccccc3c3ccccc32)ccc1-c1ccc(-n2c3ccccc3c3c4oc5ccccc5c4ccc32)cc1C#N ; N#Cc1cc(-c2ccc(-n3c4ccccc4c4ccc5c6ccccc6oc5c43)c(C#N)c2)ccc1-n1c2ccccc2c2ccccc21 ; N#Cc1cc(-c2ccc(-n3c4ccccc4c4ccc5oc6ccccc6c5c43)c(C#N)c2)ccc1-n1c2ccccc2c2ccccc21]

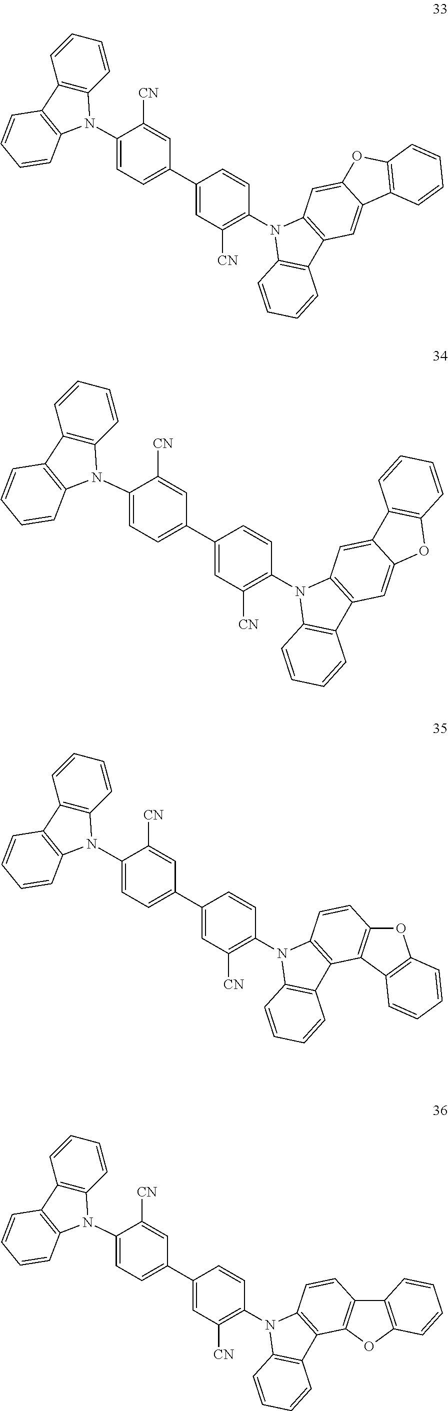 [SMILES: N#Cc1cc(-c2ccc(-n3c4ccccc4c4cc5c(cc43)oc3ccccc35)c(C#N)c2)ccc1-n1c2ccccc2c2ccccc21 ; N#Cc1cc(-c2ccc(-n3c4ccccc4c4cc5oc6ccccc6c5cc43)c(C#N)c2)ccc1-n1c2ccccc2c2ccccc21 ; N#Cc1cc(-c2ccc(-n3c4ccccc4c4c5c(ccc43)oc3ccccc35)c(C#N)c2)ccc1-n1c2ccccc2c2ccccc21 ; N#Cc1cc(-c2ccc(-n3c4ccccc4c4c5oc6ccccc6c5ccc43)c(C#N)c2)ccc1-n1c2ccccc2c2ccccc21]

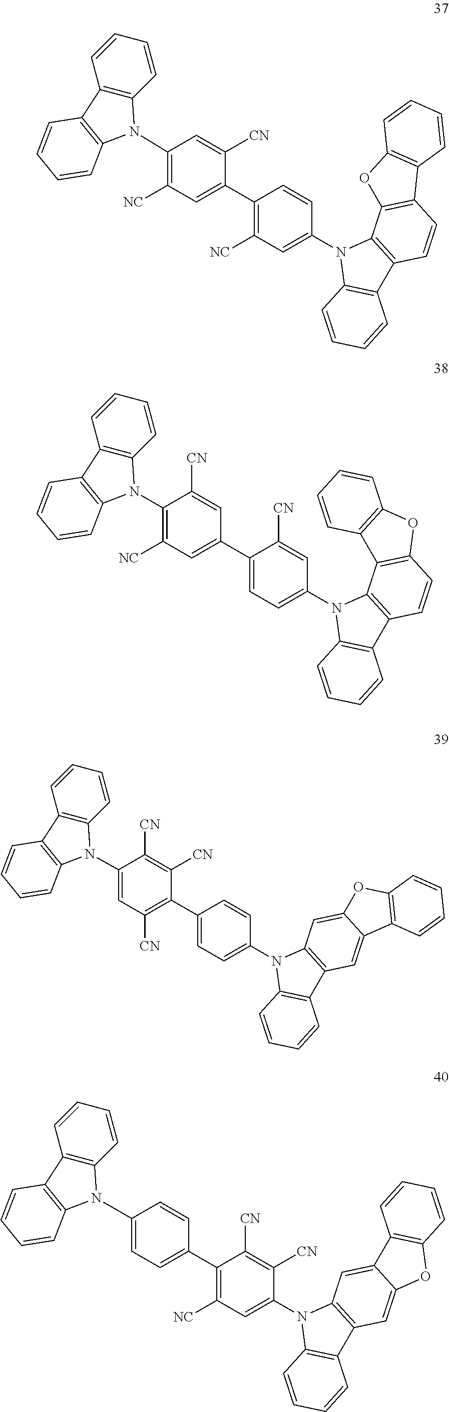 [SMILES: N#Cc1cc(-n2c3ccccc3c3ccc4c5ccccc5oc4c32)ccc1-c1cc(C#N)c(-n2c3ccccc3c3ccccc32)cc1C#N ; N#Cc1cc(-n2c3ccccc3c3ccc4oc5ccccc5c4c32)ccc1-c1cc(C#N)c(-n2c3ccccc3c3ccccc32)c(C#N)c1 ; N#Cc1cc(-n2c3ccccc3c3ccccc32)c(C#N)c(C#N)c1-c1ccc(-n2c3ccccc3c3cc4c(cc32)oc2ccccc24)cc1 ; N#Cc1cc(-n2c3ccccc3c3cc4oc5ccccc5c4cc32)c(C#N)c(C#N)c1-c1ccc(-n2c3ccccc3c3ccccc32)cc1]

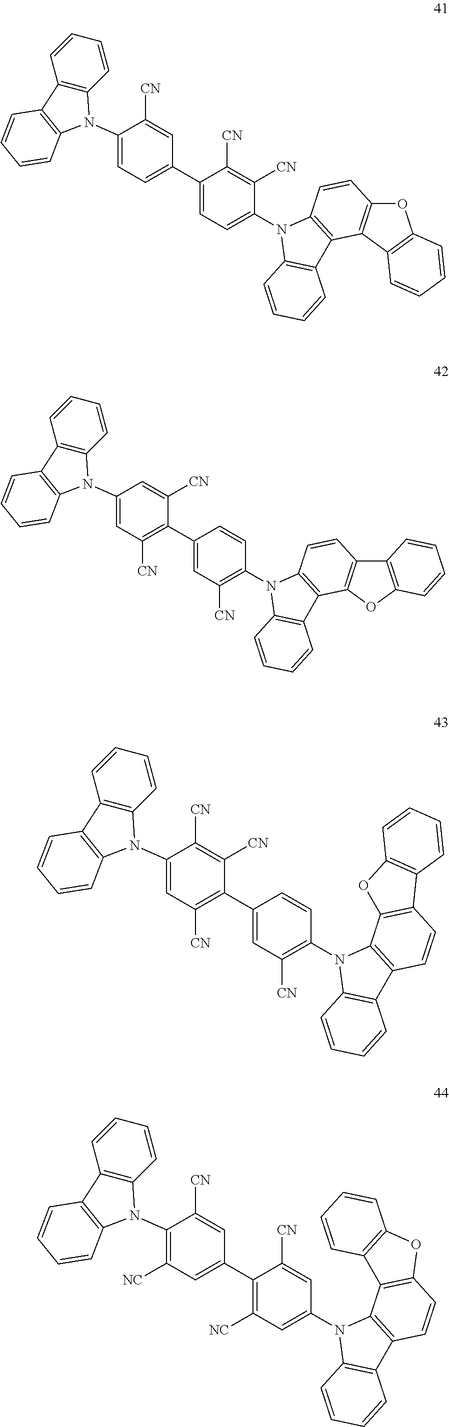 [SMILES: N#Cc1cc(-c2ccc(-n3c4ccccc4c4c5c(ccc43)oc3ccccc35)c(C#N)c2C#N)ccc1-n1c2ccccc2c2ccccc21 ; N#Cc1cc(-c2c(C#N)cc(-n3c4ccccc4c4ccccc43)cc2C#N)ccc1-n1c2ccccc2c2c3oc4ccccc4c3ccc21 ; N#Cc1cc(-c2c(C#N)cc(-n3c4ccccc4c4ccccc43)c(C#N)c2C#N)ccc1-n1c2ccccc2c2ccc3c4ccccc4oc3c21 ; N#Cc1cc(-n2c3ccccc3c3ccc4oc5ccccc5c4c32)cc(C#N)c1-c1cc(C#N)c(-n2c3ccccc3c3ccccc32)c(C#N)c1]

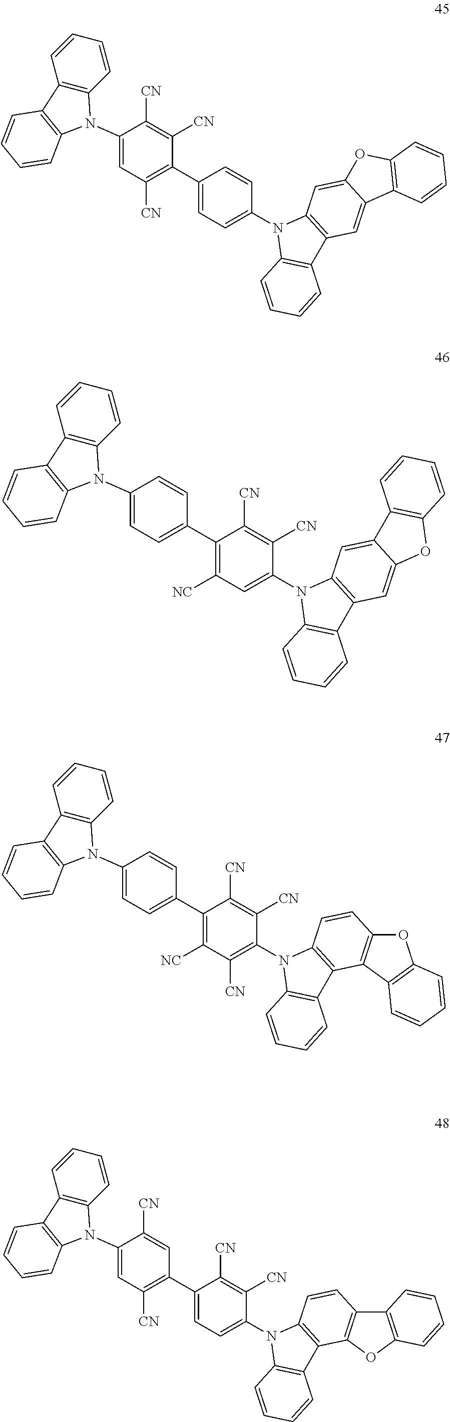 [SMILES: N#Cc1cc(-n2c3ccccc3c3ccccc32)c(C#N)c(C#N)c1-c1ccc(-n2c3ccccc3c3cc4c(cc32)oc2ccccc24)cc1 ; N#Cc1cc(-n2c3ccccc3c3cc4oc5ccccc5c4cc32)c(C#N)c(C#N)c1-c1ccc(-n2c3ccccc3c3ccccc32)cc1 ; N#Cc1c(C#N)c(-n2c3ccccc3c3c4c(ccc32)oc2ccccc24)c(C#N)c(C#N)c1-c1ccc(-n2c3ccccc3c3ccccc32)cc1 ; N#Cc1cc(-n2c3ccccc3c3ccccc32)c(C#N)cc1-c1ccc(-n2c3ccccc3c3c4oc5ccccc5c4ccc32)c(C#N)c1C#N]

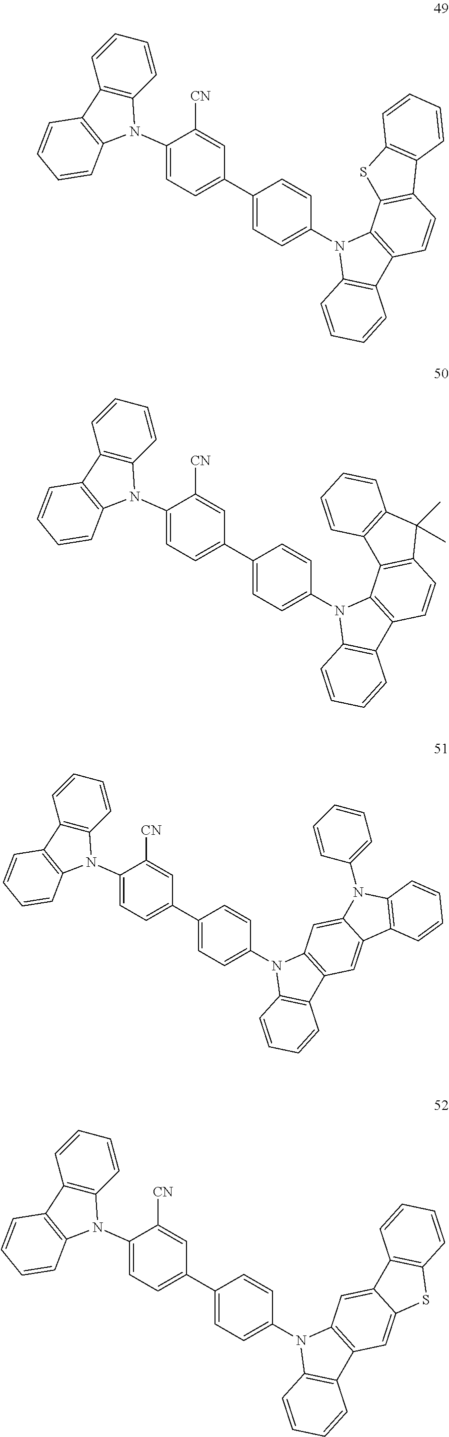 [SMILES: N#Cc1cc(-c2ccc(-n3c4ccccc4c4ccc5c6ccccc6sc5c43)cc2)ccc1-n1c2ccccc2c2ccccc21 ; CC1(C)c2ccccc2-c2c1ccc1c3ccccc3n(-c3ccc(-c4ccc(-n5c6ccccc6c6ccccc65)c(C#N)c4)cc3)c21 ; N#Cc1cc(-c2ccc(-n3c4ccccc4c4cc5c6ccccc6n(-c6ccccc6)c5cc43)cc2)ccc1-n1c2ccccc2c2ccccc21 ; N#Cc1cc(-c2ccc(-n3c4ccccc4c4cc5sc6ccccc6c5cc43)cc2)ccc1-n1c2ccccc2c2ccccc21]

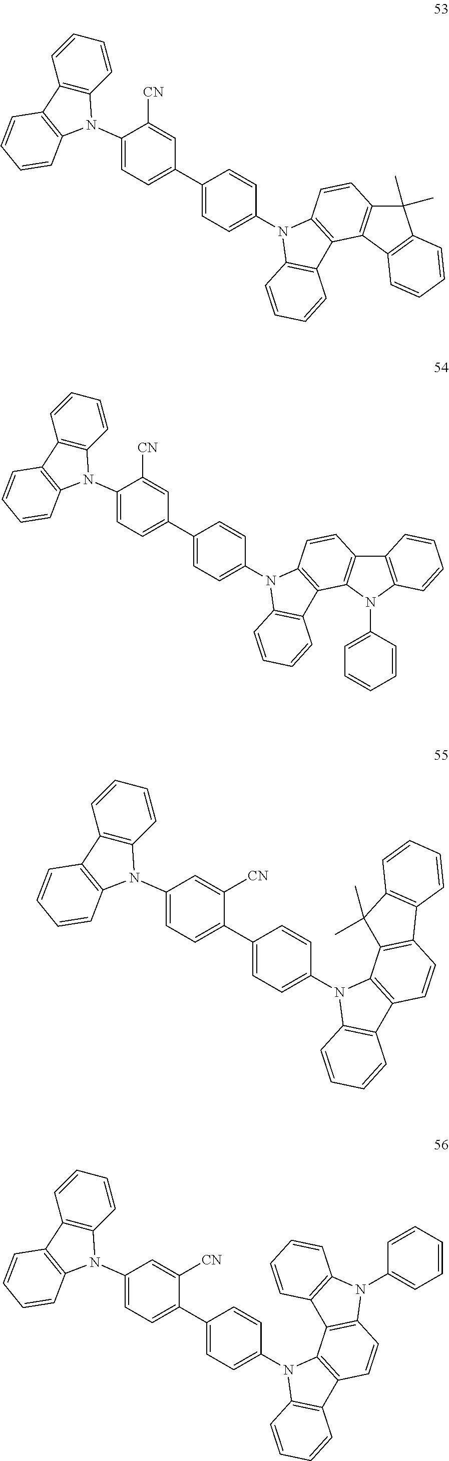 [SMILES: CC1(C)c2ccccc2-c2c1ccc1c2c2ccccc2n1-c1ccc(-c2ccc(-n3c4ccccc4c4ccccc43)c(C#N)c2)cc1 ; N#Cc1cc(-c2ccc(-n3c4ccccc4c4c3ccc3c5ccccc5n(-c5ccccc5)c34)cc2)ccc1-n1c2ccccc2c2ccccc21 ; CC1(C)c2ccccc2-c2ccc3c4ccccc4n(-c4ccc(-c5ccc(-n6c7ccccc7c7ccccc76)cc5C#N)cc4)c3c21 ; N#Cc1cc(-n2c3ccccc3c3ccccc32)ccc1-c1ccc(-n2c3ccccc3c3ccc4c(c5ccccc5n4-c4ccccc4)c32)cc1]

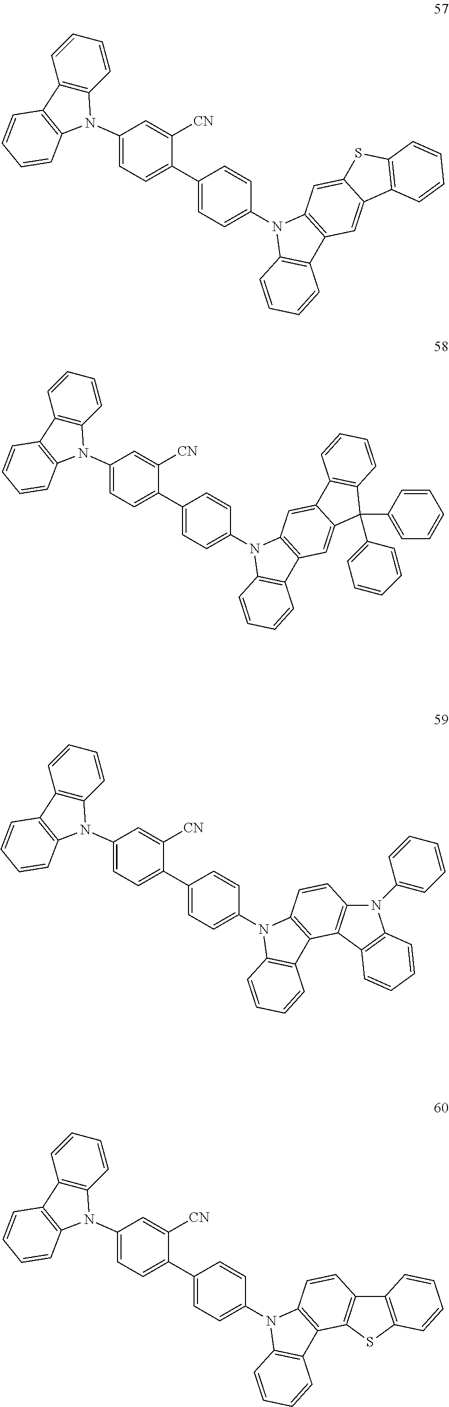 [SMILES: N#Cc1cc(-n2c3ccccc3c3ccccc32)ccc1-c1ccc(-n2c3ccccc3c3cc4c(cc32)sc2ccccc24)cc1 ; N#Cc1cc(-n2c3ccccc3c3ccccc32)ccc1-c1ccc(-n2c3ccccc3c3cc4c(cc32)-c2ccccc2C4(c2ccccc2)c2ccccc2)cc1 ; N#Cc1cc(-n2c3ccccc3c3ccccc32)ccc1-c1ccc(-n2c3ccccc3c3c4c5ccccc5n(-c5ccccc5)c4ccc32)cc1 ; N#Cc1cc(-n2c3ccccc3c3ccccc32)ccc1-c1ccc(-n2c3ccccc3c3c4sc5ccccc5c4ccc32)cc1]

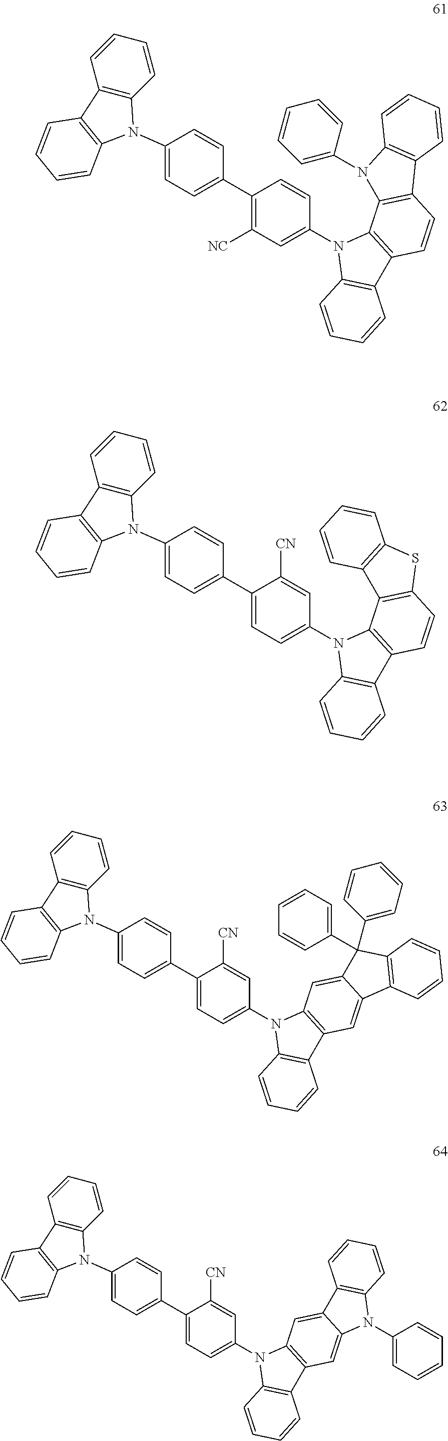 [SMILES: N#Cc1cc(-n2c3ccccc3c3ccc4c5ccccc5n(-c5ccccc5)c4c32)ccc1-c1ccc(-n2c3ccccc3c3ccccc32)cc1 ; N#Cc1cc(-n2c3ccccc3c3ccc4sc5ccccc5c4c32)ccc1-c1ccc(-n2c3ccccc3c3ccccc32)cc1 ; N#Cc1cc(-n2c3ccccc3c3cc4c(cc32)C(c2ccccc2)(c2ccccc2)c2ccccc2-4)ccc1-c1ccc(-n2c3ccccc3c3ccccc32)cc1 ; N#Cc1cc(-n2c3ccccc3c3cc4c(cc32)c2ccccc2n4-c2ccccc2)ccc1-c1ccc(-n2c3ccccc3c3ccccc32)cc1]

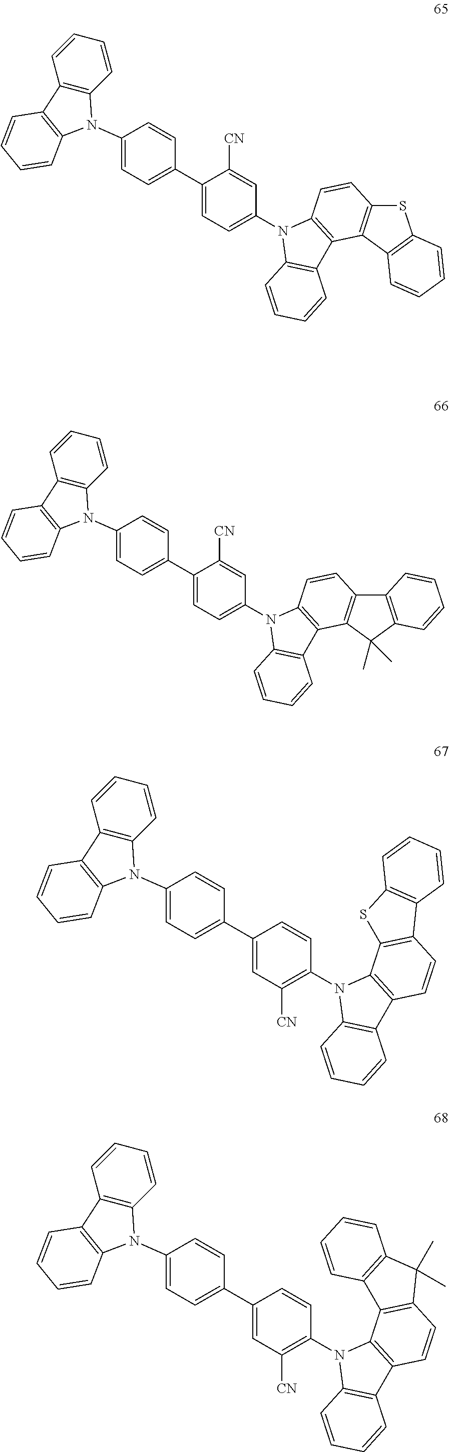 [SMILES: N#Cc1cc(-n2c3ccccc3c3c4c(ccc32)sc2ccccc24)ccc1-c1ccc(-n2c3ccccc3c3ccccc32)cc1 ; CC1(C)c2ccccc2-c2ccc3c(c21)c1ccccc1n3-c1ccc(-c2ccc(-n3c4ccccc4c4ccccc43)cc2)c(C#N)c1 ; N#Cc1cc(-c2ccc(-n3c4ccccc4c4ccccc43)cc2)ccc1-n1c2ccccc2c2ccc3c4ccccc4sc3c21 ; CC1(C)c2ccccc2-c2c1ccc1c3ccccc3n(-c3ccc(-c4ccc(-n5c6ccccc6c6ccccc65)cc4)cc3C#N)c21]

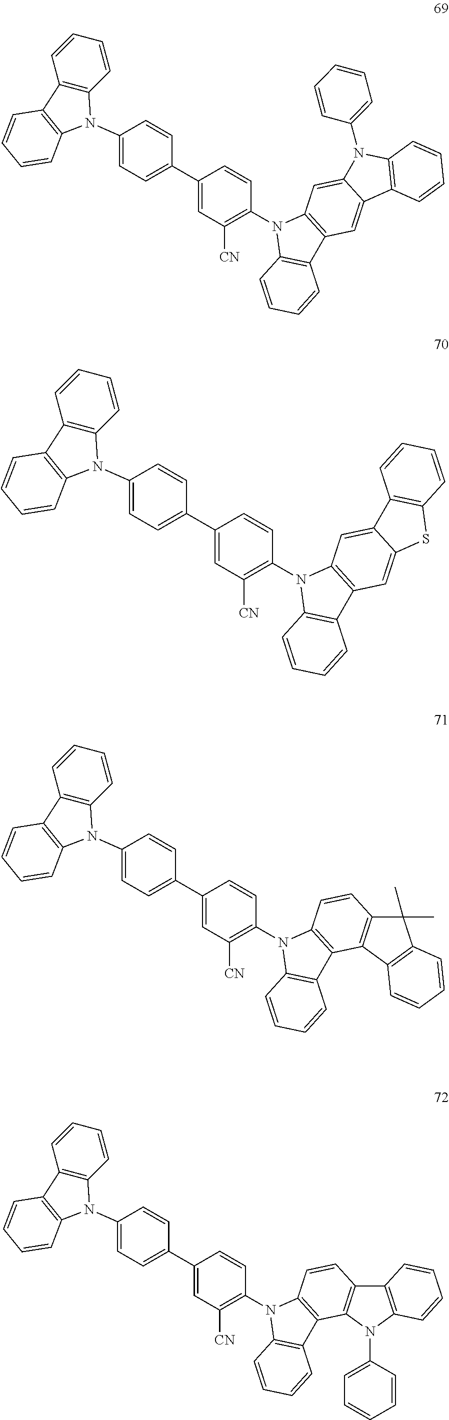 [SMILES: N#Cc1cc(-c2ccc(-n3c4ccccc4c4ccccc43)cc2)ccc1-n1c2ccccc2c2cc3c4ccccc4n(-c4ccccc4)c3cc21 ; N#Cc1cc(-c2ccc(-n3c4ccccc4c4ccccc43)cc2)ccc1-n1c2ccccc2c2cc3sc4ccccc4c3cc21 ; CC1(C)c2ccccc2-c2c1ccc1c2c2ccccc2n1-c1ccc(-c2ccc(-n3c4ccccc4c4ccccc43)cc2)cc1C#N ; N#Cc1cc(-c2ccc(-n3c4ccccc4c4ccccc43)cc2)ccc1-n1c2ccccc2c2c1ccc1c3ccccc3n(-c3ccccc3)c12]

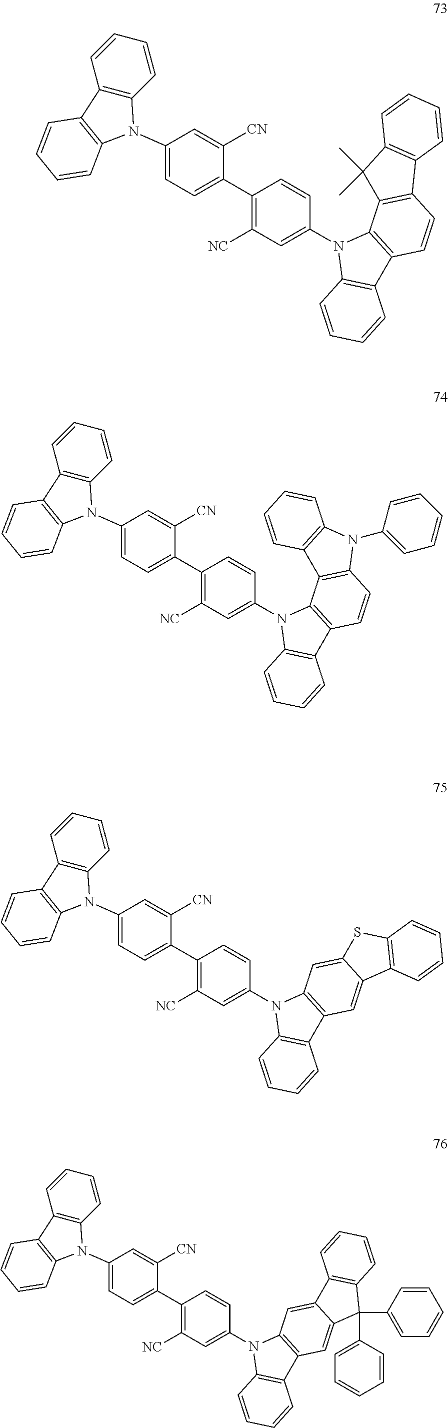 [SMILES: CC1(C)c2ccccc2-c2ccc3c4ccccc4n(-c4ccc(-c5ccc(-n6c7ccccc7c7ccccc76)cc5C#N)c(C#N)c4)c3c21 ; N#Cc1cc(-n2c3ccccc3c3ccccc32)ccc1-c1ccc(-n2c3ccccc3c3ccc4c(c5ccccc5n4-c4ccccc4)c32)cc1C#N ; N#Cc1cc(-n2c3ccccc3c3ccccc32)ccc1-c1ccc(-n2c3ccccc3c3cc4c(cc32)sc2ccccc24)cc1C#N ; N#Cc1cc(-n2c3ccccc3c3ccccc32)ccc1-c1ccc(-n2c3ccccc3c3cc4c(cc32)-c2ccccc2C4(c2ccccc2)c2ccccc2)cc1C#N]

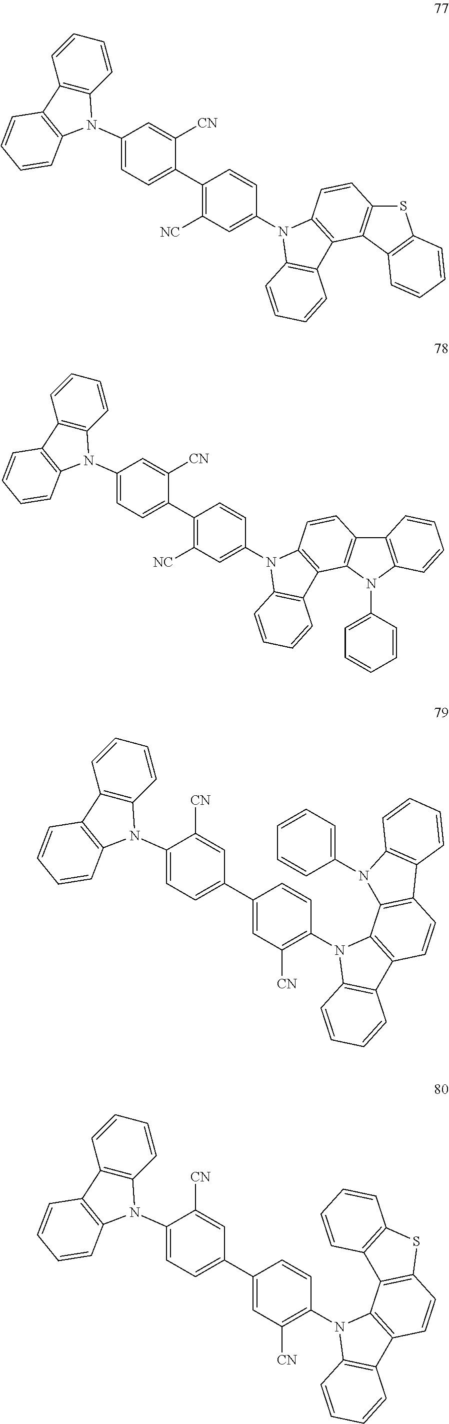 [SMILES: N#Cc1cc(-n2c3ccccc3c3ccccc32)ccc1-c1ccc(-n2c3ccccc3c3c4c(ccc32)sc2ccccc24)cc1C#N ; N#Cc1cc(-n2c3ccccc3c3ccccc32)ccc1-c1ccc(-n2c3ccccc3c3c2ccc2c4ccccc4n(-c4ccccc4)c23)cc1C#N ; N#Cc1cc(-c2ccc(-n3c4ccccc4c4ccc5c6ccccc6n(-c6ccccc6)c5c43)c(C#N)c2)ccc1-n1c2ccccc2c2ccccc21 ; N#Cc1cc(-c2ccc(-n3c4ccccc4c4ccc5sc6ccccc6c5c43)c(C#N)c2)ccc1-n1c2ccccc2c2ccccc21]

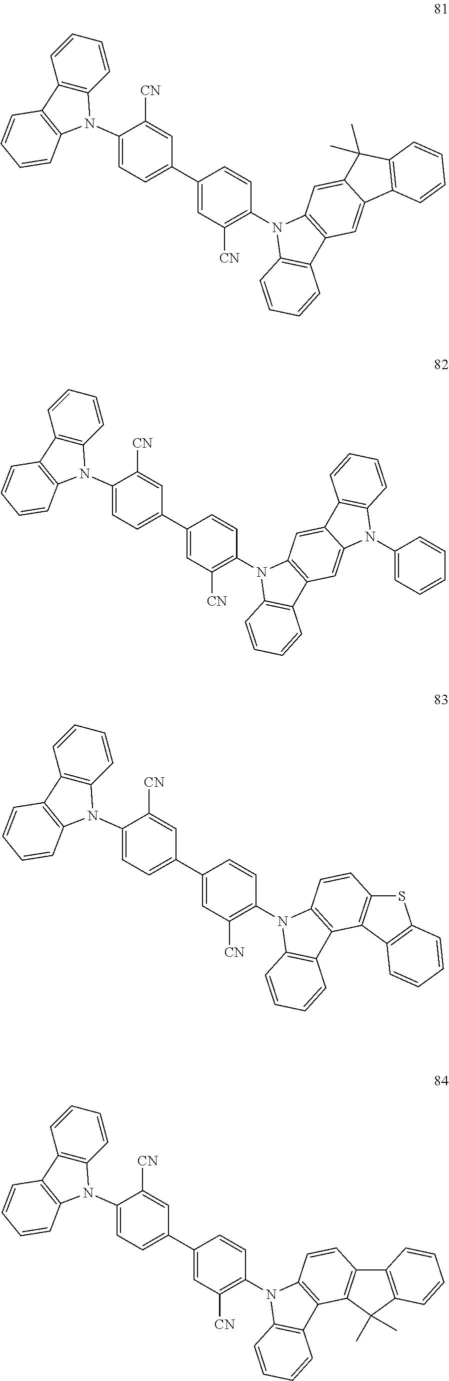 [SMILES: CC1(C)c2ccccc2-c2cc3c4ccccc4n(-c4ccc(-c5ccc(-n6c7ccccc7c7ccccc76)c(C#N)c5)cc4C#N)c3cc21 ; N#Cc1cc(-c2ccc(-n3c4ccccc4c4cc5c(cc43)c3ccccc3n5-c3ccccc3)c(C#N)c2)ccc1-n1c2ccccc2c2ccccc21 ; N#Cc1cc(-c2ccc(-n3c4ccccc4c4c5c(ccc43)sc3ccccc35)c(C#N)c2)ccc1-n1c2ccccc2c2ccccc21 ; CC1(C)c2ccccc2-c2ccc3c(c21)c1ccccc1n3-c1ccc(-c2ccc(-n3c4ccccc4c4ccccc43)c(C#N)c2)cc1C#N]

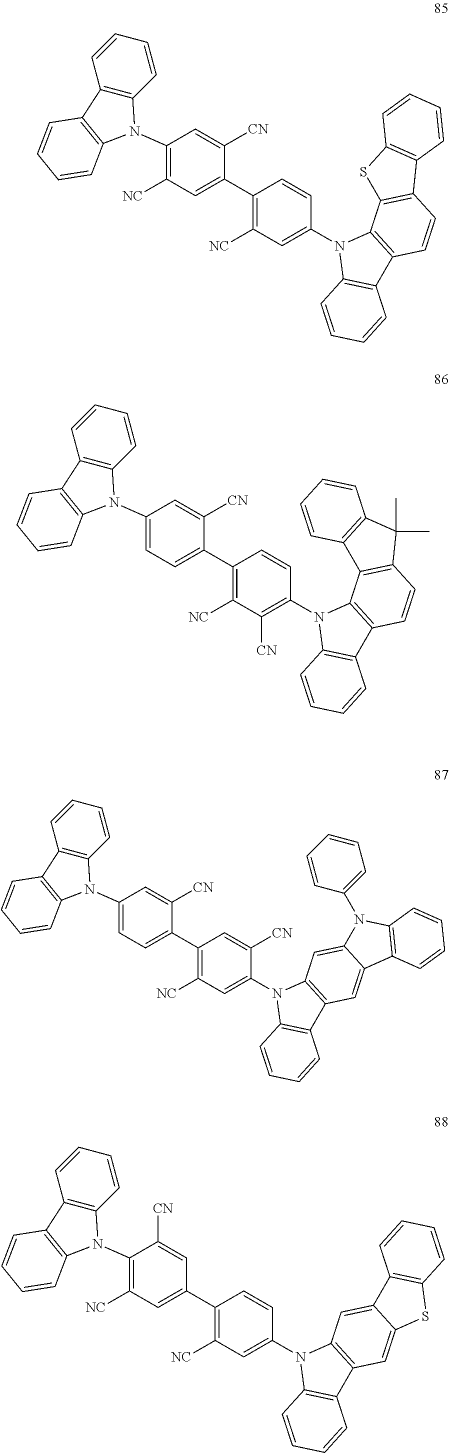 [SMILES: N#Cc1cc(-n2c3ccccc3c3ccc4c5ccccc5sc4c32)ccc1-c1cc(C#N)c(-n2c3ccccc3c3ccccc32)cc1C#N ; CC1(C)c2ccccc2-c2c1ccc1c3ccccc3n(-c3ccc(-c4ccc(-n5c6ccccc6c6ccccc65)cc4C#N)c(C#N)c3C#N)c21 ; N#Cc1cc(-n2c3ccccc3c3ccccc32)ccc1-c1cc(C#N)c(-n2c3ccccc3c3cc4c5ccccc5n(-c5ccccc5)c4cc32)cc1C#N ; N#Cc1cc(-n2c3ccccc3c3cc4sc5ccccc5c4cc32)ccc1-c1cc(C#N)c(-n2c3ccccc3c3ccccc32)c(C#N)c1]

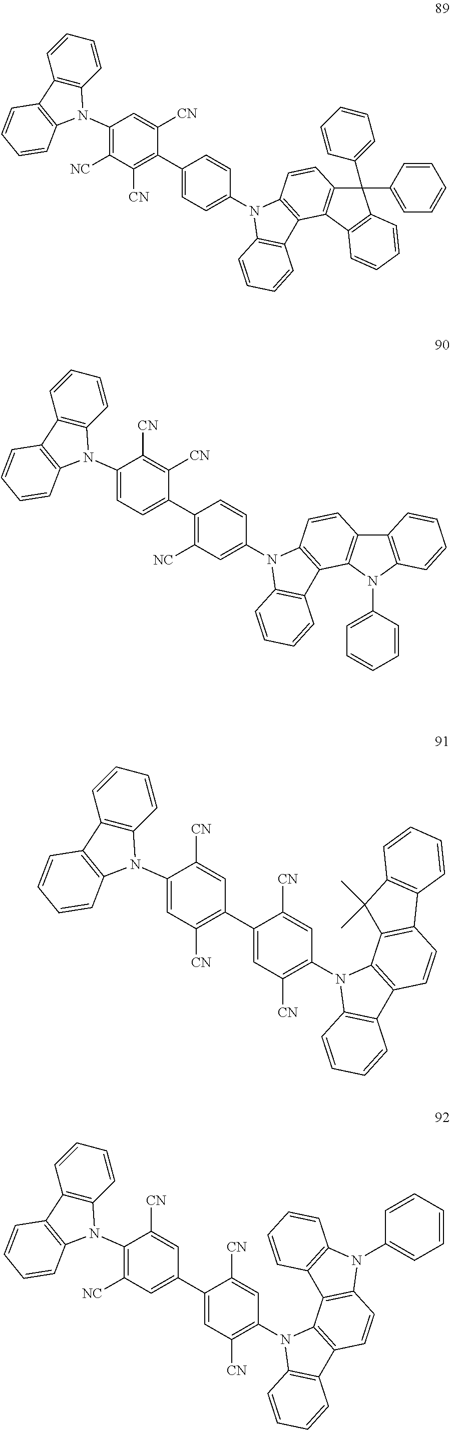 [SMILES: N#Cc1cc(-n2c3ccccc3c3ccccc32)c(C#N)c(C#N)c1-c1ccc(-n2c3ccccc3c3c4c(ccc32)C(c2ccccc2)(c2ccccc2)c2ccccc2-4)cc1 ; N#Cc1cc(-n2c3ccccc3c3c2ccc2c4ccccc4n(-c4ccccc4)c23)ccc1-c1ccc(-n2c3ccccc3c3ccccc32)c(C#N)c1C#N ; CC1(C)c2ccccc2-c2ccc3c4ccccc4n(-c4cc(C#N)c(-c5cc(C#N)c(-n6c7ccccc7c7ccccc76)cc5C#N)cc4C#N)c3c21 ; N#Cc1cc(-n2c3ccccc3c3ccc4c(c5ccccc5n4-c4ccccc4)c32)c(C#N)cc1-c1cc(C#N)c(-n2c3ccccc3c3ccccc32)c(C#N)c1]

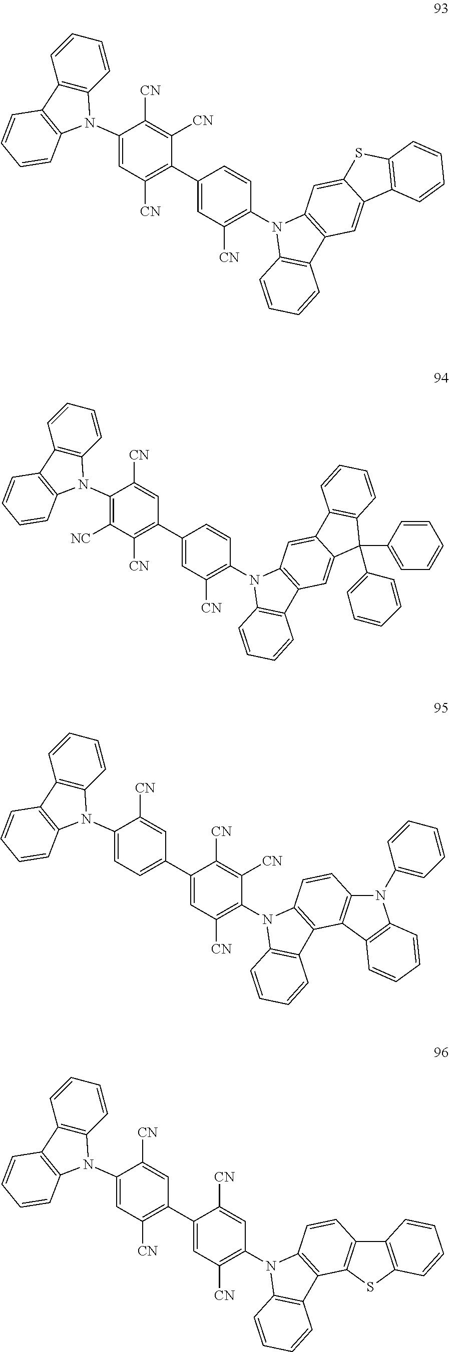 [SMILES: N#Cc1cc(-c2c(C#N)cc(-n3c4ccccc4c4ccccc43)c(C#N)c2C#N)ccc1-n1c2ccccc2c2cc3c(cc21)sc1ccccc13 ; N#Cc1cc(-c2cc(C#N)c(-n3c4ccccc4c4ccccc43)c(C#N)c2C#N)ccc1-n1c2ccccc2c2cc3c(cc21)-c1ccccc1C3(c1ccccc1)c1ccccc1 ; N#Cc1cc(-c2cc(C#N)c(-n3c4ccccc4c4c5c6ccccc6n(-c6ccccc6)c5ccc43)c(C#N)c2C#N)ccc1-n1c2ccccc2c2ccccc21 ; N#Cc1cc(-n2c3ccccc3c3ccccc32)c(C#N)cc1-c1cc(C#N)c(-n2c3ccccc3c3c4sc5ccccc5c4ccc32)cc1C#N]

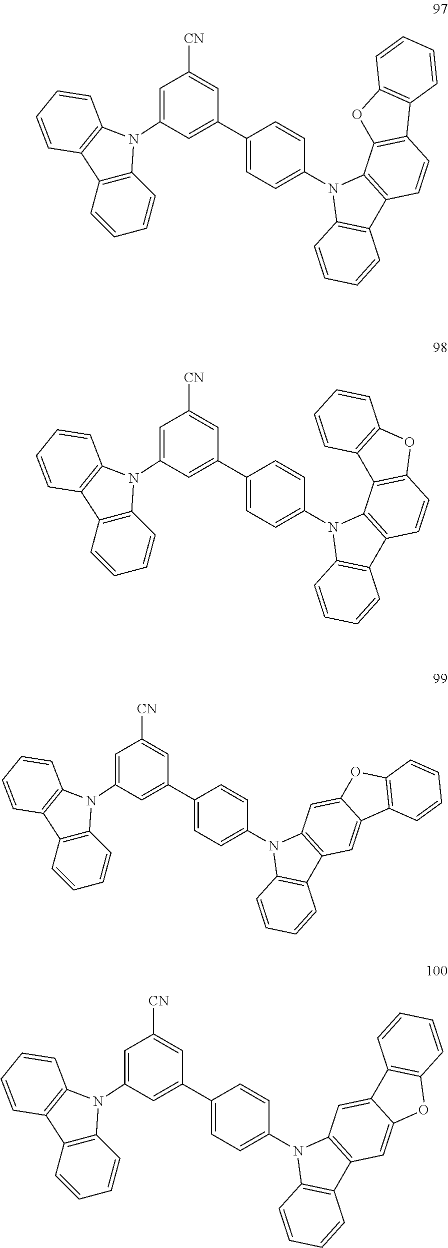 [SMILES: N#Cc1cc(-c2ccc(-n3c4ccccc4c4ccc5c6ccccc6oc5c43)cc2)cc(-n2c3ccccc3c3ccccc32)c1 ; N#Cc1cc(-c2ccc(-n3c4ccccc4c4ccc5oc6ccccc6c5c43)cc2)cc(-n2c3ccccc3c3ccccc32)c1 ; N#Cc1cc(-c2ccc(-n3c4ccccc4c4cc5c(cc43)oc3ccccc35)cc2)cc(-n2c3ccccc3c3ccccc32)c1 ; N#Cc1cc(-c2ccc(-n3c4ccccc4c4cc5oc6ccccc6c5cc43)cc2)cc(-n2c3ccccc3c3ccccc32)c1]

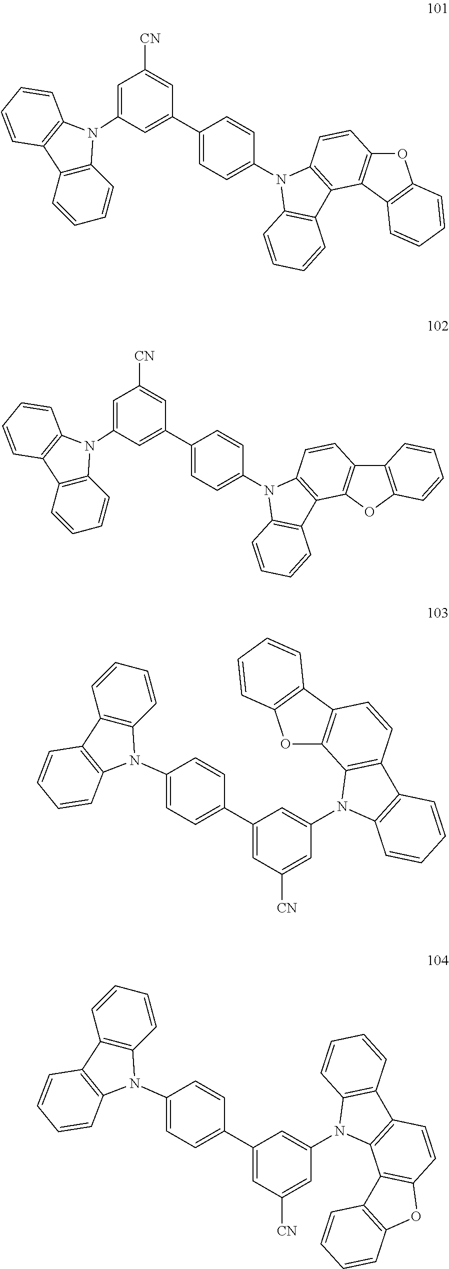 [SMILES: N#Cc1cc(-c2ccc(-n3c4ccccc4c4c5c(ccc43)oc3ccccc35)cc2)cc(-n2c3ccccc3c3ccccc32)c1 ; N#Cc1cc(-c2ccc(-n3c4ccccc4c4c5oc6ccccc6c5ccc43)cc2)cc(-n2c3ccccc3c3ccccc32)c1 ; N#Cc1cc(-c2ccc(-n3c4ccccc4c4ccccc43)cc2)cc(-n2c3ccccc3c3ccc4c5ccccc5oc4c32)c1 ; N#Cc1cc(-c2ccc(-n3c4ccccc4c4ccccc43)cc2)cc(-n2c3ccccc3c3ccc4oc5ccccc5c4c32)c1]

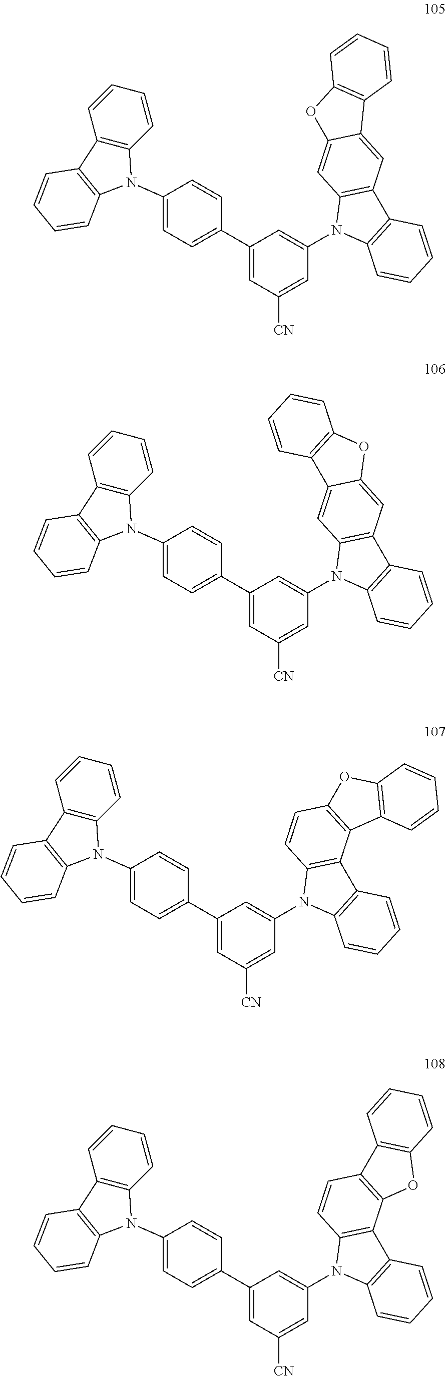 [SMILES: N#Cc1cc(-c2ccc(-n3c4ccccc4c4ccccc43)cc2)cc(-n2c3ccccc3c3cc4c(cc32)oc2ccccc24)c1 ; N#Cc1cc(-c2ccc(-n3c4ccccc4c4ccccc43)cc2)cc(-n2c3ccccc3c3cc4oc5ccccc5c4cc32)c1 ; N#Cc1cc(-c2ccc(-n3c4ccccc4c4ccccc43)cc2)cc(-n2c3ccccc3c3c4c(ccc32)oc2ccccc24)c1 ; N#Cc1cc(-c2ccc(-n3c4ccccc4c4ccccc43)cc2)cc(-n2c3ccccc3c3c4oc5ccccc5c4ccc32)c1]

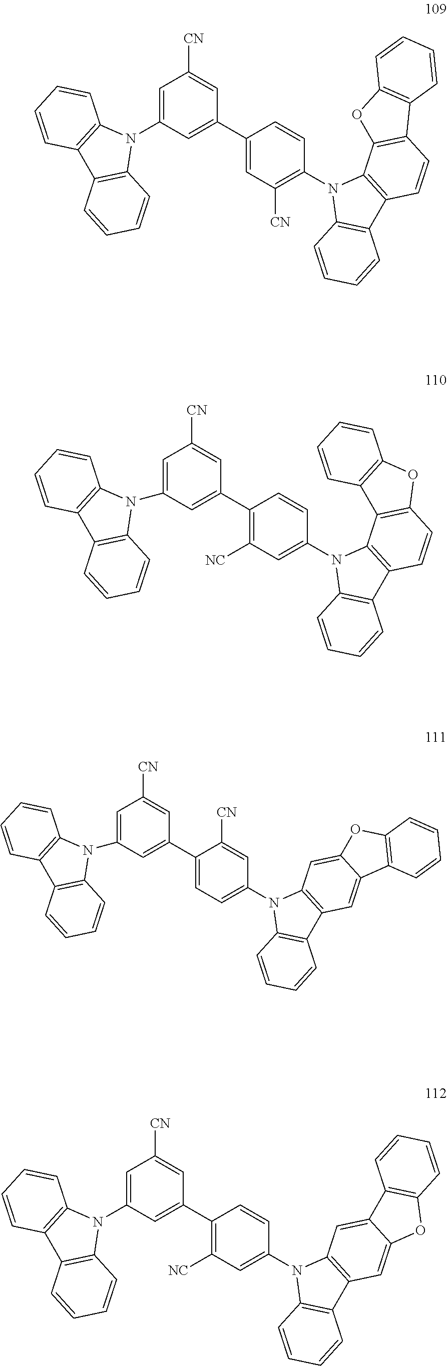 [SMILES: N#Cc1cc(-c2ccc(-n3c4ccccc4c4ccc5c6ccccc6oc5c43)c(C#N)c2)cc(-n2c3ccccc3c3ccccc32)c1 ; N#Cc1cc(-c2ccc(-n3c4ccccc4c4ccc5oc6ccccc6c5c43)cc2C#N)cc(-n2c3ccccc3c3ccccc32)c1 ; N#Cc1cc(-c2ccc(-n3c4ccccc4c4cc5c(cc43)oc3ccccc35)cc2C#N)cc(-n2c3ccccc3c3ccccc32)c1 ; N#Cc1cc(-c2ccc(-n3c4ccccc4c4cc5oc6ccccc6c5cc43)cc2C#N)cc(-n2c3ccccc3c3ccccc32)c1]

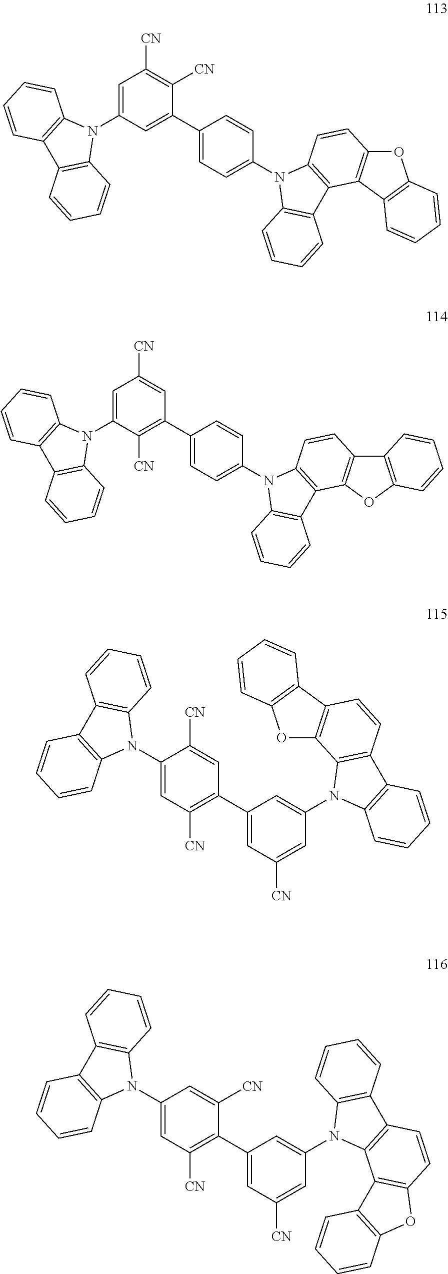 [SMILES: N#Cc1cc(-n2c3ccccc3c3ccccc32)cc(-c2ccc(-n3c4ccccc4c4c5c(ccc43)oc3ccccc35)cc2)c1C#N ; N#Cc1cc(-c2ccc(-n3c4ccccc4c4c5oc6ccccc6c5ccc43)cc2)c(C#N)c(-n2c3ccccc3c3ccccc32)c1 ; N#Cc1cc(-c2cc(C#N)c(-n3c4ccccc4c4ccccc43)cc2C#N)cc(-n2c3ccccc3c3ccc4c5ccccc5oc4c32)c1 ; N#Cc1cc(-c2c(C#N)cc(-n3c4ccccc4c4ccccc43)cc2C#N)cc(-n2c3ccccc3c3ccc4oc5ccccc5c4c32)c1]

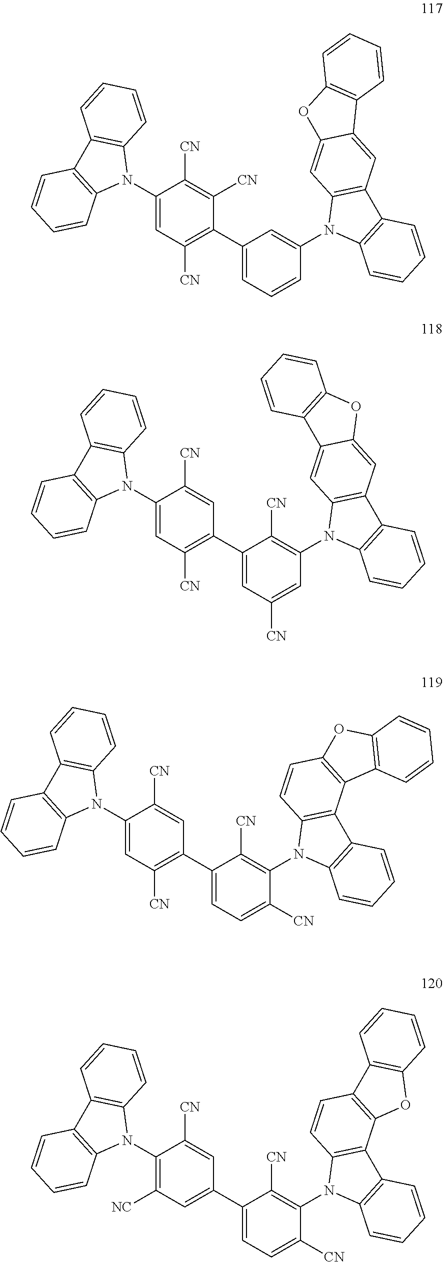 [SMILES: N#Cc1cc(-n2c3ccccc3c3ccccc32)c(C#N)c(C#N)c1-c1cccc(-n2c3ccccc3c3cc4c(cc32)oc2ccccc24)c1 ; N#Cc1cc(-c2cc(C#N)c(-n3c4ccccc4c4ccccc43)cc2C#N)c(C#N)c(-n2c3ccccc3c3cc4oc5ccccc5c4cc32)c1 ; N#Cc1cc(-n2c3ccccc3c3ccccc32)c(C#N)cc1-c1ccc(C#N)c(-n2c3ccccc3c3c4c(ccc32)oc2ccccc24)c1C#N ; N#Cc1cc(-c2ccc(C#N)c(-n3c4ccccc4c4c5oc6ccccc6c5ccc43)c2C#N)cc(C#N)c1-n1c2ccccc2c2ccccc21]

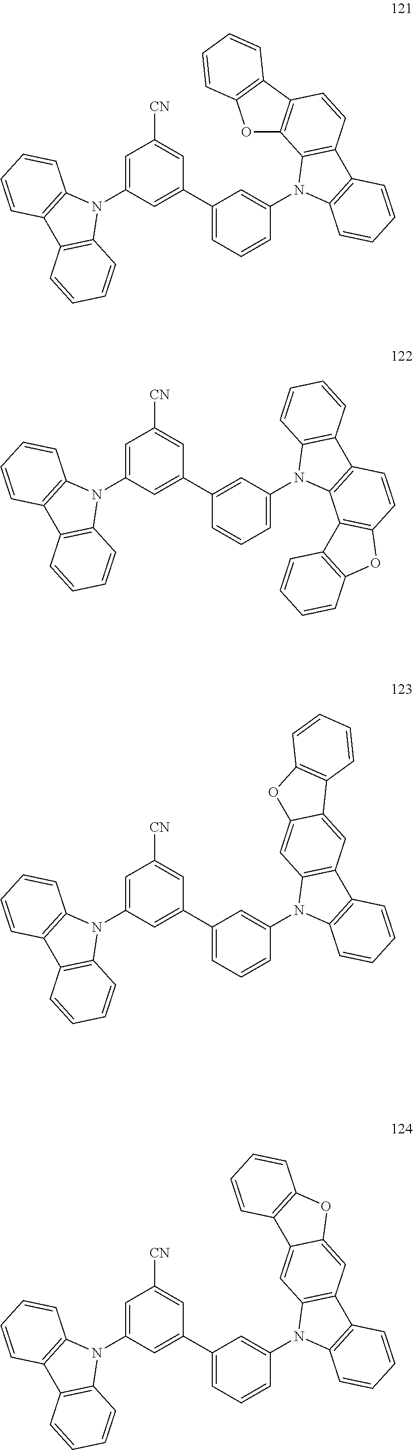 [SMILES: N#Cc1cc(-c2cccc(-n3c4ccccc4c4ccc5c6ccccc6oc5c43)c2)cc(-n2c3ccccc3c3ccccc32)c1 ; N#Cc1cc(-c2cccc(-n3c4ccccc4c4ccc5oc6ccccc6c5c43)c2)cc(-n2c3ccccc3c3ccccc32)c1 ; N#Cc1cc(-c2cccc(-n3c4ccccc4c4cc5c(cc43)oc3ccccc35)c2)cc(-n2c3ccccc3c3ccccc32)c1 ; N#Cc1cc(-c2cccc(-n3c4ccccc4c4cc5oc6ccccc6c5cc43)c2)cc(-n2c3ccccc3c3ccccc32)c1]

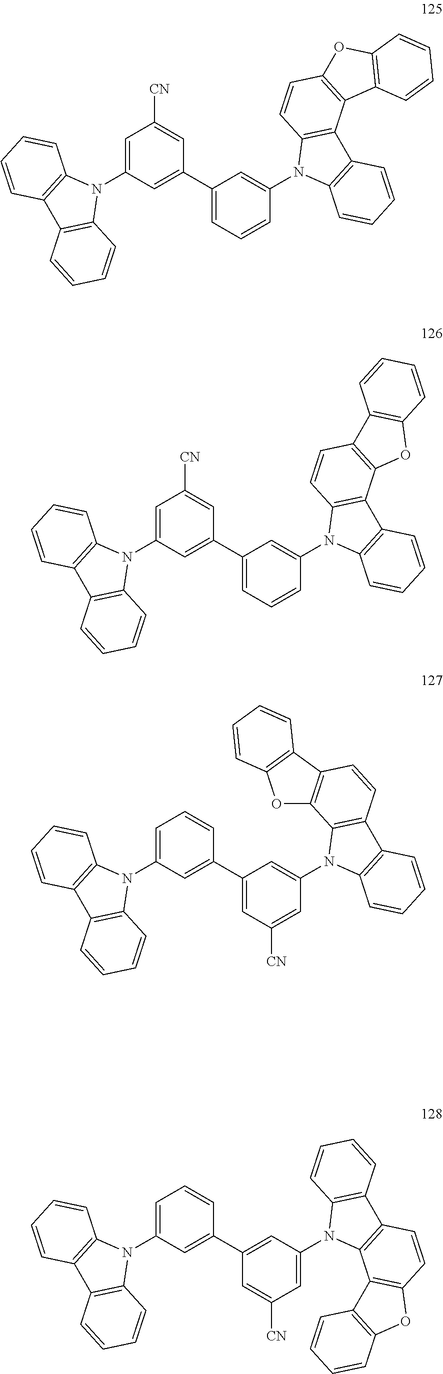 [SMILES: N#Cc1cc(-c2cccc(-n3c4ccccc4c4c5c(ccc43)oc3ccccc35)c2)cc(-n2c3ccccc3c3ccccc32)c1 ; N#Cc1cc(-c2cccc(-n3c4ccccc4c4c5oc6ccccc6c5ccc43)c2)cc(-n2c3ccccc3c3ccccc32)c1 ; N#Cc1cc(-c2cccc(-n3c4ccccc4c4ccccc43)c2)cc(-n2c3ccccc3c3ccc4c5ccccc5oc4c32)c1 ; N#Cc1cc(-c2cccc(-n3c4ccccc4c4ccccc43)c2)cc(-n2c3ccccc3c3ccc4oc5ccccc5c4c32)c1]

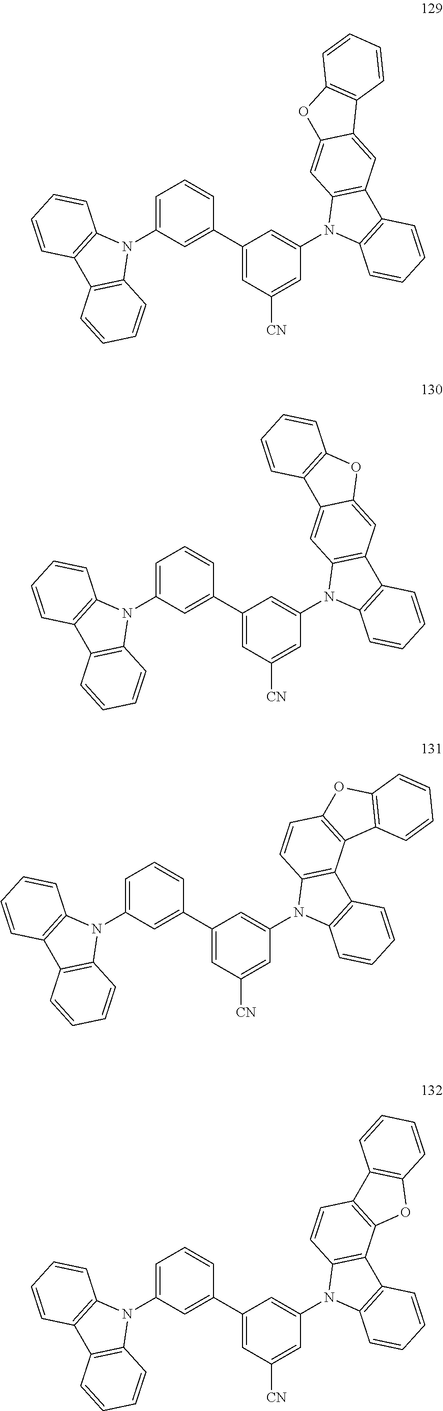 [SMILES: N#Cc1cc(-c2cccc(-n3c4ccccc4c4ccccc43)c2)cc(-n2c3ccccc3c3cc4c(cc32)oc2ccccc24)c1 ; N#Cc1cc(-c2cccc(-n3c4ccccc4c4ccccc43)c2)cc(-n2c3ccccc3c3cc4oc5ccccc5c4cc32)c1 ; N#Cc1cc(-c2cccc(-n3c4ccccc4c4ccccc43)c2)cc(-n2c3ccccc3c3c4c(ccc32)oc2ccccc24)c1 ; N#Cc1cc(-c2cccc(-n3c4ccccc4c4ccccc43)c2)cc(-n2c3ccccc3c3c4oc5ccccc5c4ccc32)c1]

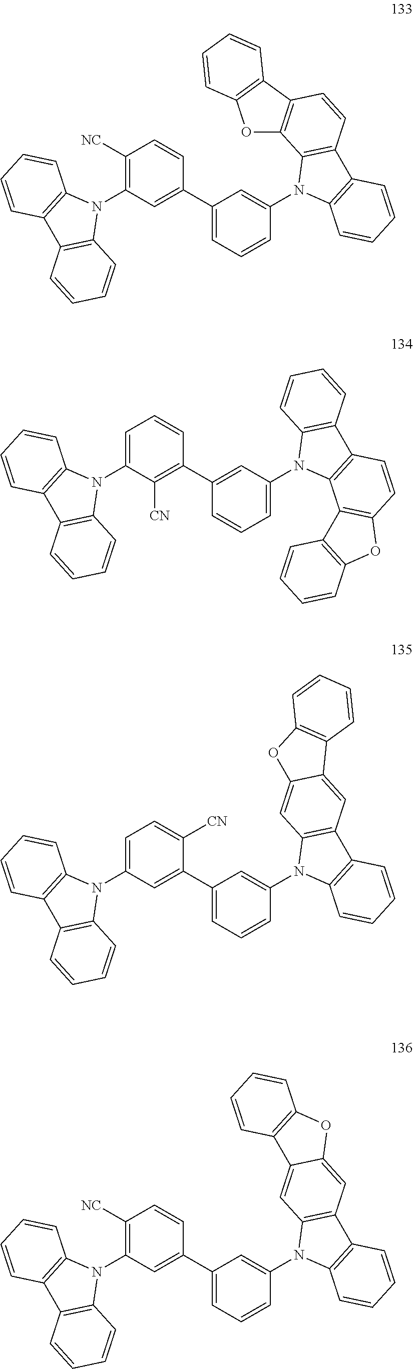 [SMILES: N#Cc1ccc(-c2cccc(-n3c4ccccc4c4ccc5c6ccccc6oc5c43)c2)cc1-n1c2ccccc2c2ccccc21 ; N#Cc1c(-c2cccc(-n3c4ccccc4c4ccc5oc6ccccc6c5c43)c2)cccc1-n1c2ccccc2c2ccccc21 ; N#Cc1ccc(-n2c3ccccc3c3ccccc32)cc1-c1cccc(-n2c3ccccc3c3cc4c(cc32)oc2ccccc24)c1 ; N#Cc1ccc(-c2cccc(-n3c4ccccc4c4cc5oc6ccccc6c5cc43)c2)cc1-n1c2ccccc2c2ccccc21]

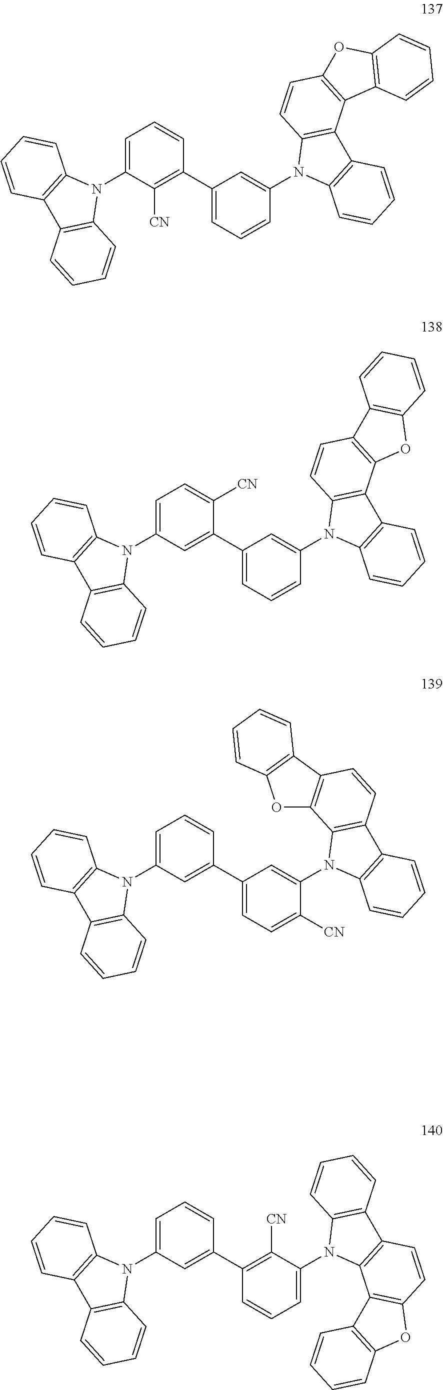 [SMILES: N#Cc1c(-c2cccc(-n3c4ccccc4c4c5c(ccc43)oc3ccccc35)c2)cccc1-n1c2ccccc2c2ccccc21 ; N#Cc1ccc(-n2c3ccccc3c3ccccc32)cc1-c1cccc(-n2c3ccccc3c3c4oc5ccccc5c4ccc32)c1 ; N#Cc1ccc(-c2cccc(-n3c4ccccc4c4ccccc43)c2)cc1-n1c2ccccc2c2ccc3c4ccccc4oc3c21 ; N#Cc1c(-c2cccc(-n3c4ccccc4c4ccccc43)c2)cccc1-n1c2ccccc2c2ccc3oc4ccccc4c3c21]

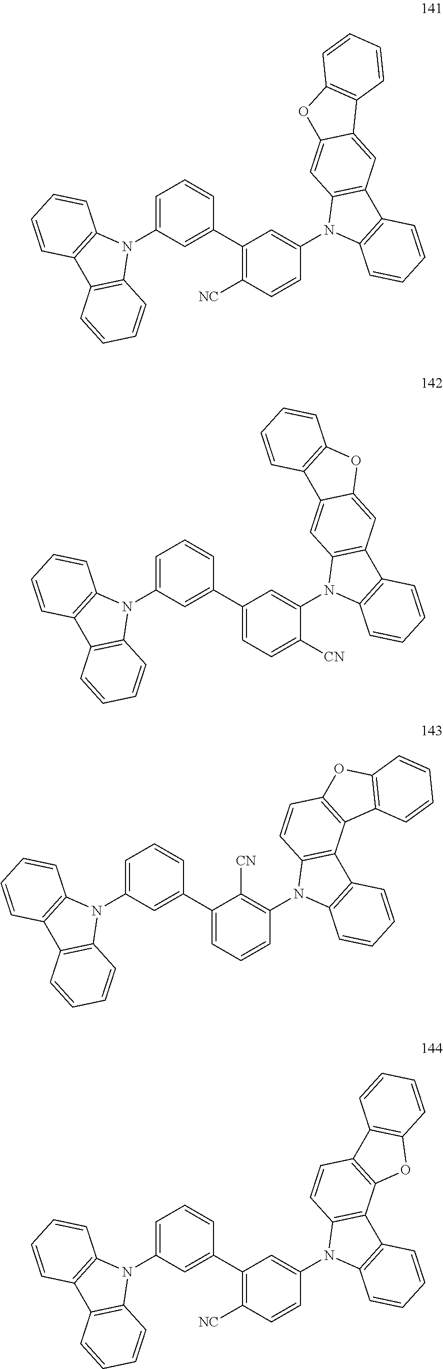 [SMILES: N#Cc1ccc(-n2c3ccccc3c3cc4c(cc32)oc2ccccc24)cc1-c1cccc(-n2c3ccccc3c3ccccc32)c1 ; N#Cc1ccc(-c2cccc(-n3c4ccccc4c4ccccc43)c2)cc1-n1c2ccccc2c2cc3oc4ccccc4c3cc21 ; N#Cc1c(-c2cccc(-n3c4ccccc4c4ccccc43)c2)cccc1-n1c2ccccc2c2c3c(ccc21)oc1ccccc13 ; N#Cc1ccc(-n2c3ccccc3c3c4oc5ccccc5c4ccc32)cc1-c1cccc(-n2c3ccccc3c3ccccc32)c1]

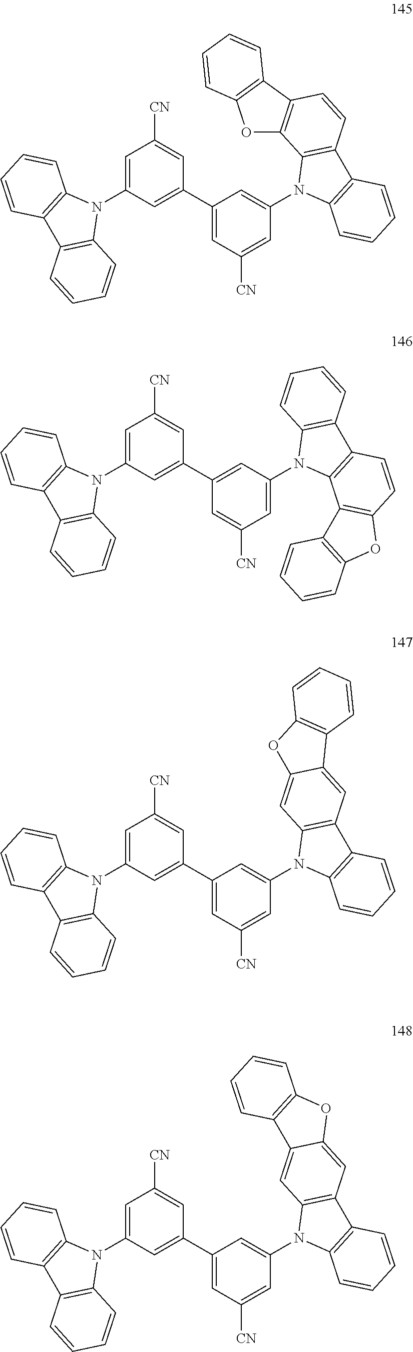 [SMILES: N#Cc1cc(-c2cc(C#N)cc(-n3c4ccccc4c4ccc5c6ccccc6oc5c43)c2)cc(-n2c3ccccc3c3ccccc32)c1 ; N#Cc1cc(-c2cc(C#N)cc(-n3c4ccccc4c4ccc5oc6ccccc6c5c43)c2)cc(-n2c3ccccc3c3ccccc32)c1 ; N#Cc1cc(-c2cc(C#N)cc(-n3c4ccccc4c4cc5c(cc43)oc3ccccc35)c2)cc(-n2c3ccccc3c3ccccc32)c1 ; N#Cc1cc(-c2cc(C#N)cc(-n3c4ccccc4c4cc5oc6ccccc6c5cc43)c2)cc(-n2c3ccccc3c3ccccc32)c1]

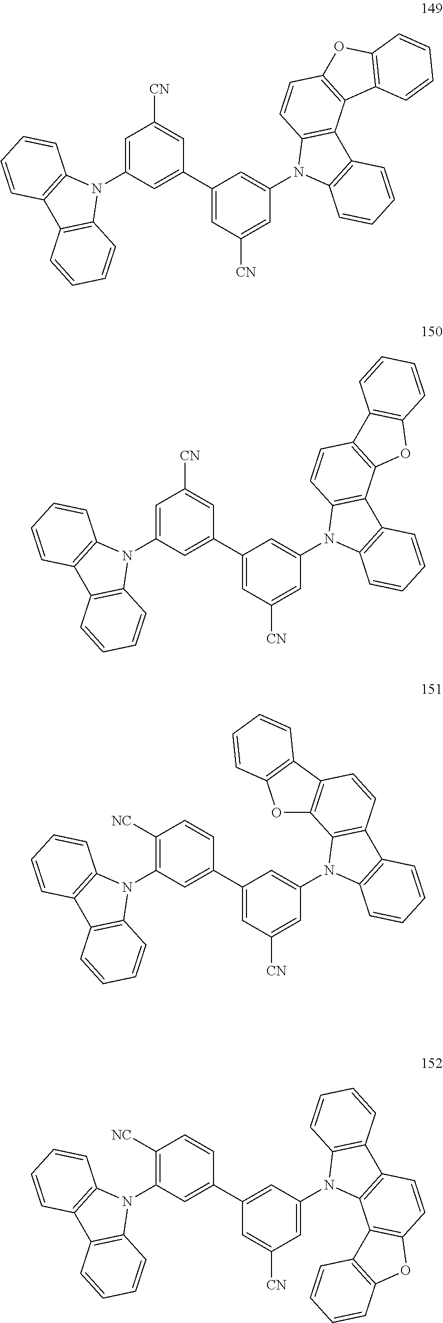 [SMILES: N#Cc1cc(-c2cc(C#N)cc(-n3c4ccccc4c4c5c(ccc43)oc3ccccc35)c2)cc(-n2c3ccccc3c3ccccc32)c1 ; N#Cc1cc(-c2cc(C#N)cc(-n3c4ccccc4c4c5oc6ccccc6c5ccc43)c2)cc(-n2c3ccccc3c3ccccc32)c1 ; N#Cc1cc(-c2ccc(C#N)c(-n3c4ccccc4c4ccccc43)c2)cc(-n2c3ccccc3c3ccc4c5ccccc5oc4c32)c1 ; N#Cc1cc(-c2ccc(C#N)c(-n3c4ccccc4c4ccccc43)c2)cc(-n2c3ccccc3c3ccc4oc5ccccc5c4c32)c1]

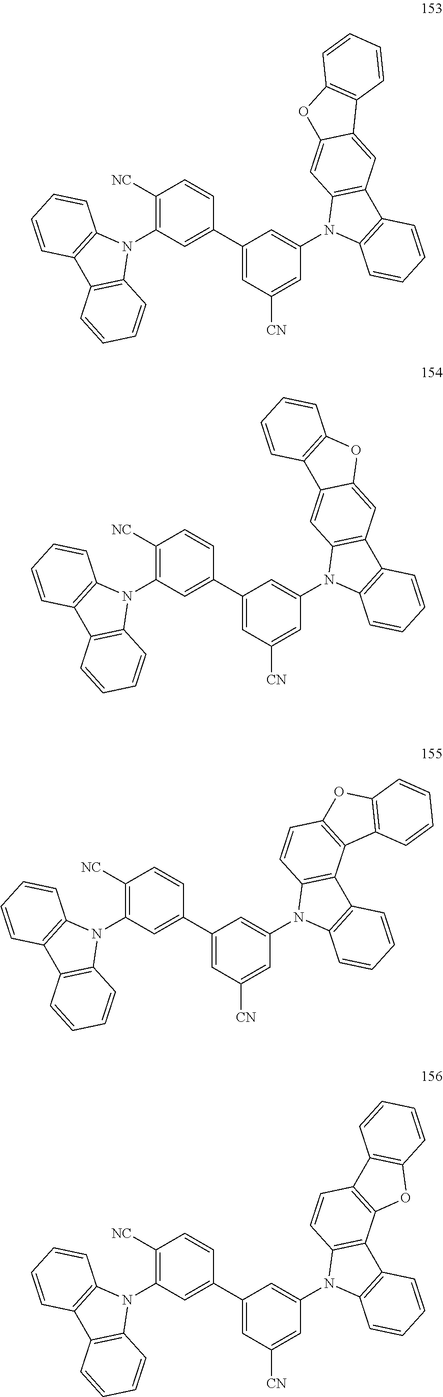 [SMILES: N#Cc1cc(-c2ccc(C#N)c(-n3c4ccccc4c4ccccc43)c2)cc(-n2c3ccccc3c3cc4c(cc32)oc2ccccc24)c1 ; N#Cc1cc(-c2ccc(C#N)c(-n3c4ccccc4c4ccccc43)c2)cc(-n2c3ccccc3c3cc4oc5ccccc5c4cc32)c1 ; N#Cc1cc(-c2ccc(C#N)c(-n3c4ccccc4c4ccccc43)c2)cc(-n2c3ccccc3c3c4c(ccc32)oc2ccccc24)c1 ; N#Cc1cc(-c2ccc(C#N)c(-n3c4ccccc4c4ccccc43)c2)cc(-n2c3ccccc3c3c4oc5ccccc5c4ccc32)c1]

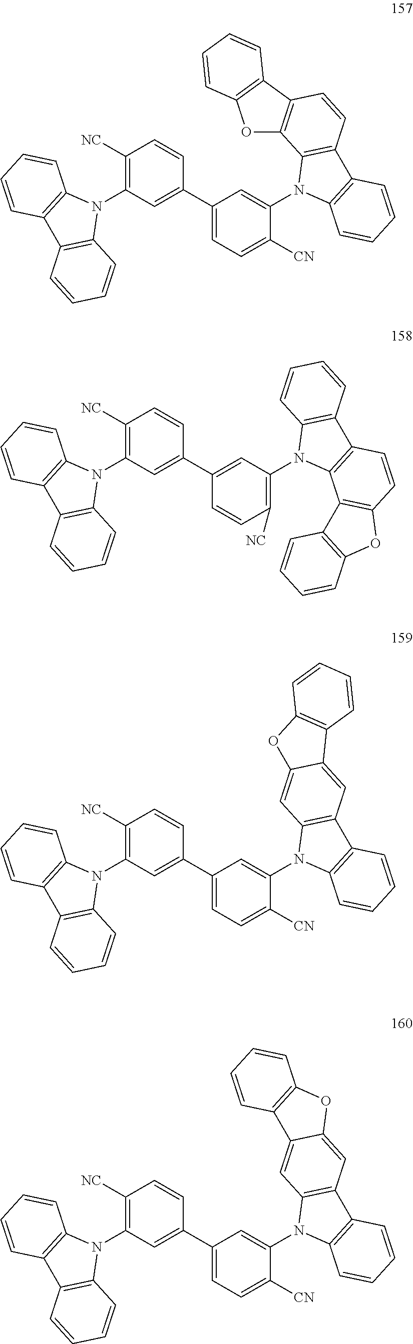 [SMILES: N#Cc1ccc(-c2ccc(C#N)c(-n3c4ccccc4c4ccc5c6ccccc6oc5c43)c2)cc1-n1c2ccccc2c2ccccc21 ; N#Cc1ccc(-c2ccc(C#N)c(-n3c4ccccc4c4ccc5oc6ccccc6c5c43)c2)cc1-n1c2ccccc2c2ccccc21 ; N#Cc1ccc(-c2ccc(C#N)c(-n3c4ccccc4c4cc5c(cc43)oc3ccccc35)c2)cc1-n1c2ccccc2c2ccccc21 ; N#Cc1ccc(-c2ccc(C#N)c(-n3c4ccccc4c4cc5oc6ccccc6c5cc43)c2)cc1-n1c2ccccc2c2ccccc21]

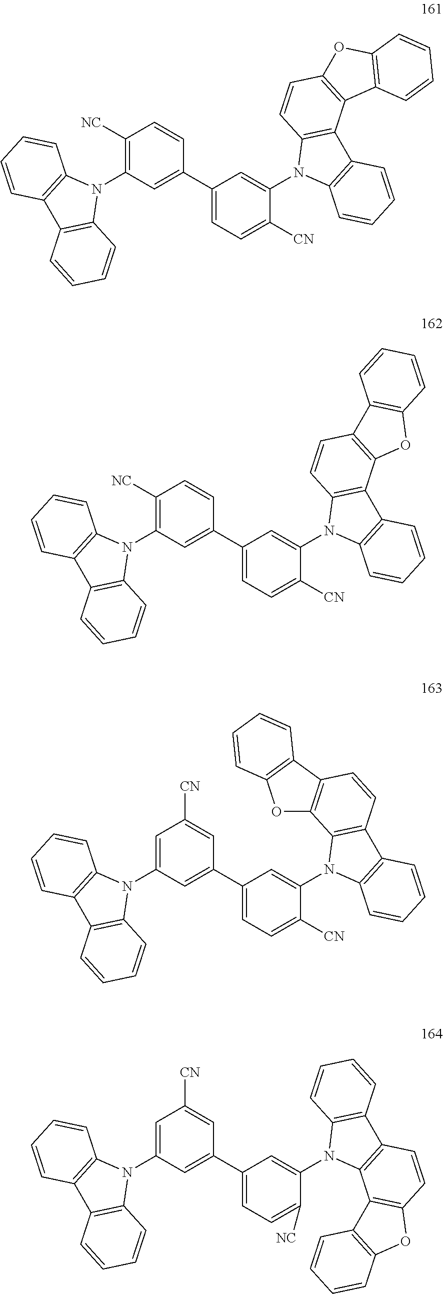 [SMILES: N#Cc1ccc(-c2ccc(C#N)c(-n3c4ccccc4c4c5c(ccc43)oc3ccccc35)c2)cc1-n1c2ccccc2c2ccccc21 ; N#Cc1ccc(-c2ccc(C#N)c(-n3c4ccccc4c4c5oc6ccccc6c5ccc43)c2)cc1-n1c2ccccc2c2ccccc21 ; N#Cc1cc(-c2ccc(C#N)c(-n3c4ccccc4c4ccc5c6ccccc6oc5c43)c2)cc(-n2c3ccccc3c3ccccc32)c1 ; N#Cc1cc(-c2ccc(C#N)c(-n3c4ccccc4c4ccc5oc6ccccc6c5c43)c2)cc(-n2c3ccccc3c3ccccc32)c1]

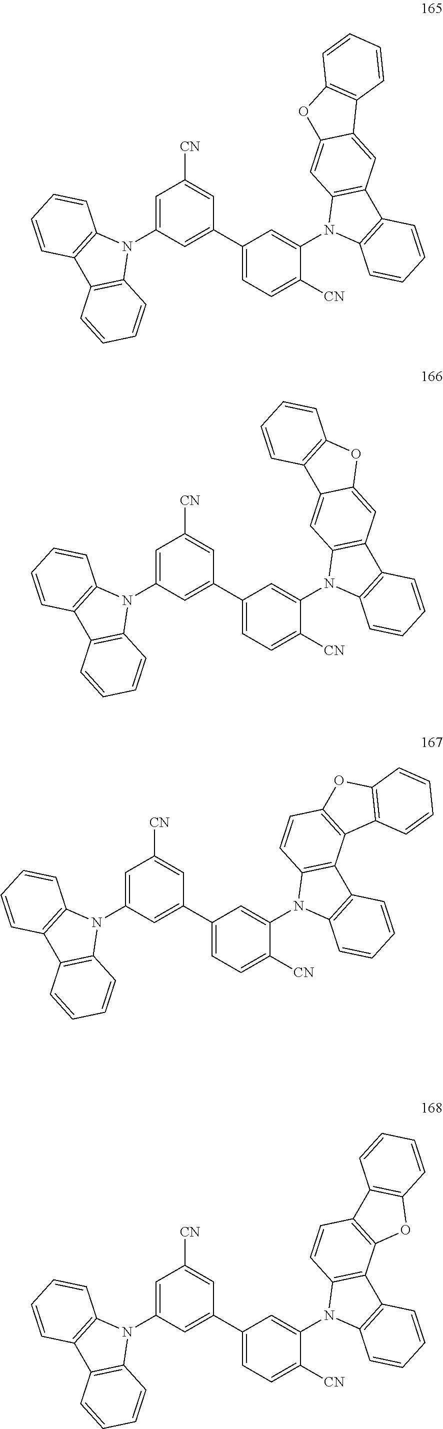 [SMILES: N#Cc1cc(-c2ccc(C#N)c(-n3c4ccccc4c4cc5c(cc43)oc3ccccc35)c2)cc(-n2c3ccccc3c3ccccc32)c1 ; N#Cc1cc(-c2ccc(C#N)c(-n3c4ccccc4c4cc5oc6ccccc6c5cc43)c2)cc(-n2c3ccccc3c3ccccc32)c1 ; N#Cc1cc(-c2ccc(C#N)c(-n3c4ccccc4c4c5c(ccc43)oc3ccccc35)c2)cc(-n2c3ccccc3c3ccccc32)c1 ; N#Cc1cc(-c2ccc(C#N)c(-n3c4ccccc4c4c5oc6ccccc6c5ccc43)c2)cc(-n2c3ccccc3c3ccccc32)c1]

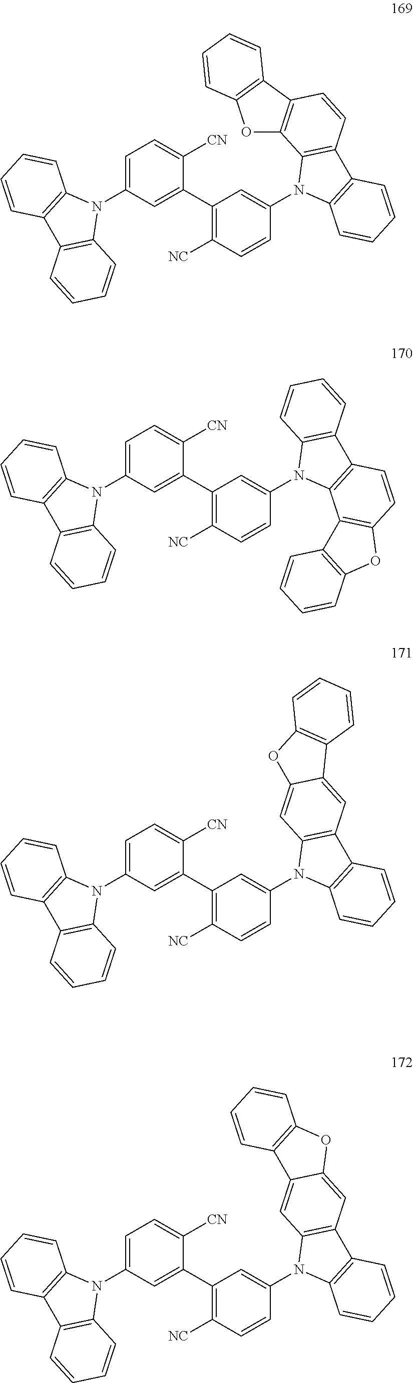 [SMILES: N#Cc1ccc(-n2c3ccccc3c3ccccc32)cc1-c1cc(-n2c3ccccc3c3ccc4c5ccccc5oc4c32)ccc1C#N ; N#Cc1ccc(-n2c3ccccc3c3ccccc32)cc1-c1cc(-n2c3ccccc3c3ccc4oc5ccccc5c4c32)ccc1C#N ; N#Cc1ccc(-n2c3ccccc3c3ccccc32)cc1-c1cc(-n2c3ccccc3c3cc4c(cc32)oc2ccccc24)ccc1C#N ; N#Cc1ccc(-n2c3ccccc3c3ccccc32)cc1-c1cc(-n2c3ccccc3c3cc4oc5ccccc5c4cc32)ccc1C#N]

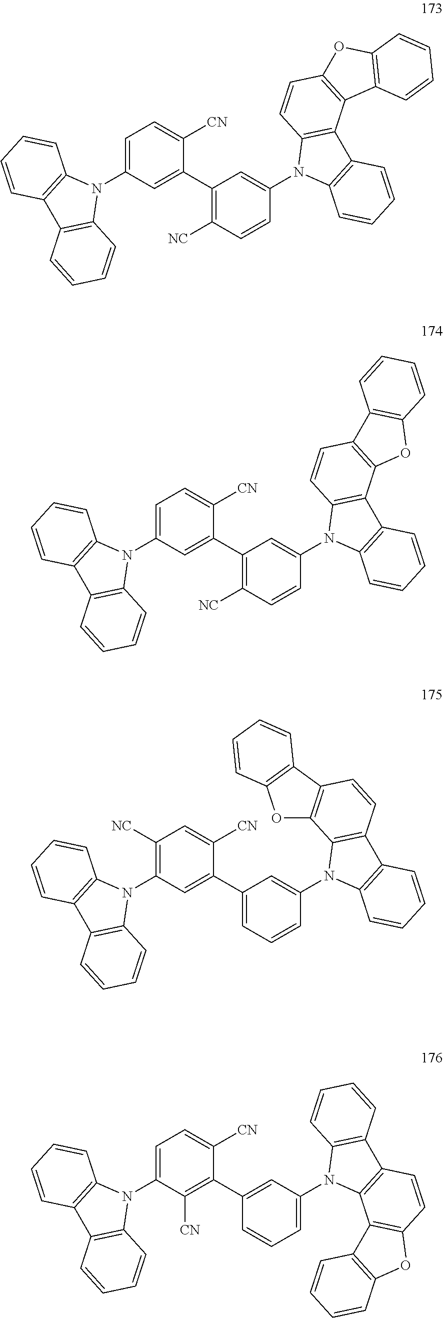 [SMILES: N#Cc1ccc(-n2c3ccccc3c3ccccc32)cc1-c1cc(-n2c3ccccc3c3c4c(ccc32)oc2ccccc24)ccc1C#N ; N#Cc1ccc(-n2c3ccccc3c3ccccc32)cc1-c1cc(-n2c3ccccc3c3c4oc5ccccc5c4ccc32)ccc1C#N ; N#Cc1cc(C#N)c(-n2c3ccccc3c3ccccc32)cc1-c1cccc(-n2c3ccccc3c3ccc4c5ccccc5oc4c32)c1 ; N#Cc1ccc(-n2c3ccccc3c3ccccc32)c(C#N)c1-c1cccc(-n2c3ccccc3c3ccc4oc5ccccc5c4c32)c1]

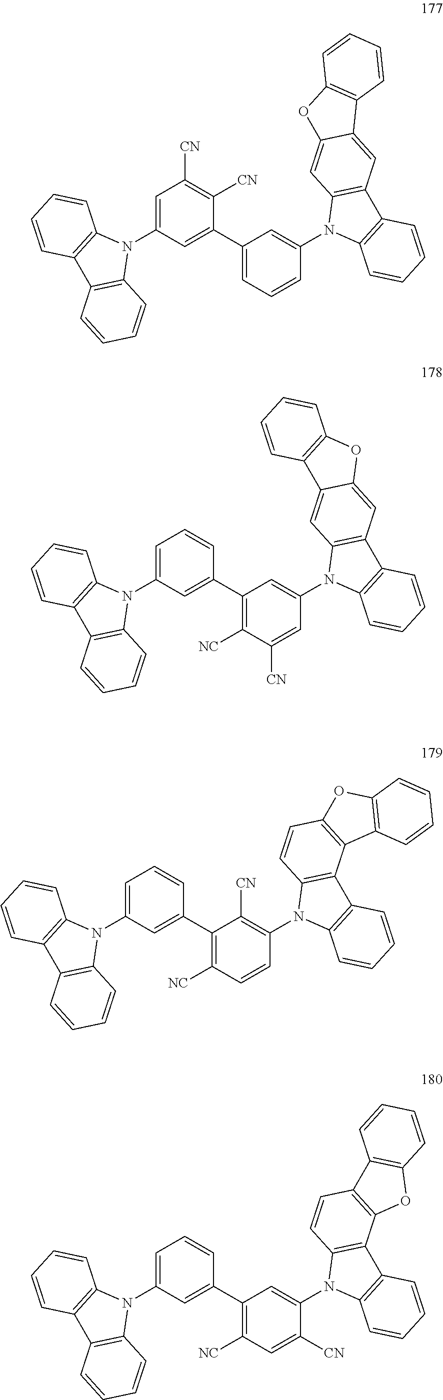 [SMILES: N#Cc1cc(-n2c3ccccc3c3ccccc32)cc(-c2cccc(-n3c4ccccc4c4cc5c(cc43)oc3ccccc35)c2)c1C#N ; N#Cc1cc(-n2c3ccccc3c3cc4oc5ccccc5c4cc32)cc(-c2cccc(-n3c4ccccc4c4ccccc43)c2)c1C#N ; N#Cc1ccc(-n2c3ccccc3c3c4c(ccc32)oc2ccccc24)c(C#N)c1-c1cccc(-n2c3ccccc3c3ccccc32)c1 ; N#Cc1cc(C#N)c(-n2c3ccccc3c3c4oc5ccccc5c4ccc32)cc1-c1cccc(-n2c3ccccc3c3ccccc32)c1]

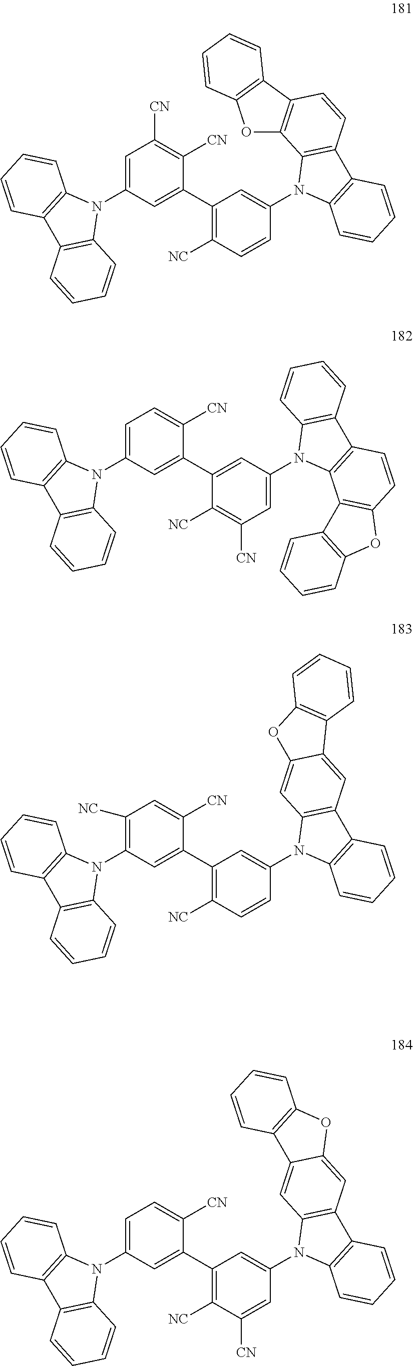 [SMILES: N#Cc1ccc(-n2c3ccccc3c3ccc4c5ccccc5oc4c32)cc1-c1cc(-n2c3ccccc3c3ccccc32)cc(C#N)c1C#N ; N#Cc1ccc(-n2c3ccccc3c3ccccc32)cc1-c1cc(-n2c3ccccc3c3ccc4oc5ccccc5c4c32)cc(C#N)c1C#N ; N#Cc1ccc(-n2c3ccccc3c3cc4c(cc32)oc2ccccc24)cc1-c1cc(-n2c3ccccc3c3ccccc32)c(C#N)cc1C#N ; N#Cc1ccc(-n2c3ccccc3c3ccccc32)cc1-c1cc(-n2c3ccccc3c3cc4oc5ccccc5c4cc32)cc(C#N)c1C#N]

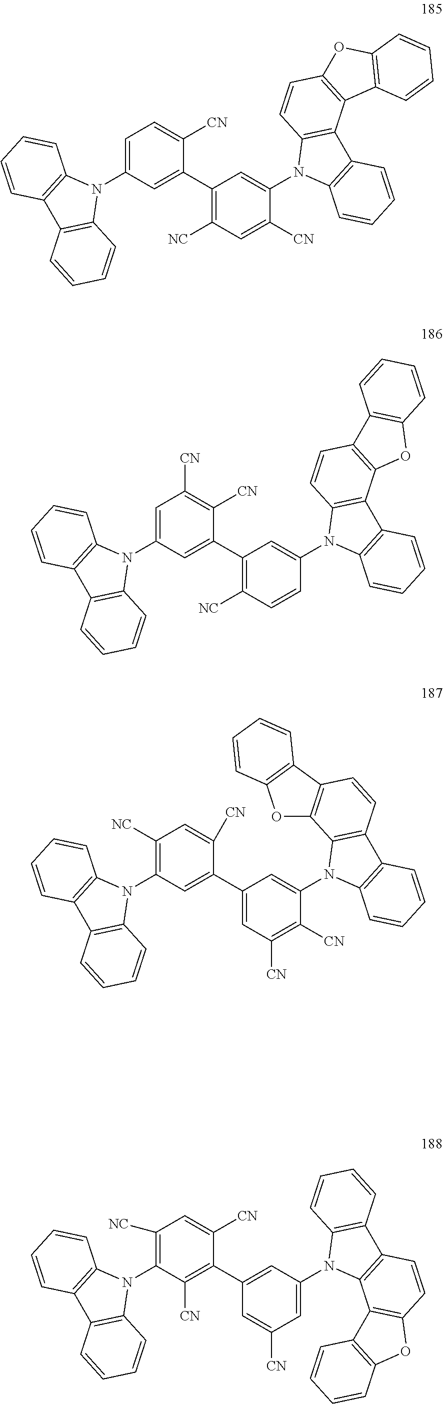 [SMILES: N#Cc1ccc(-n2c3ccccc3c3ccccc32)cc1-c1cc(-n2c3ccccc3c3c4c(ccc32)oc2ccccc24)c(C#N)cc1C#N ; N#Cc1ccc(-n2c3ccccc3c3c4oc5ccccc5c4ccc32)cc1-c1cc(-n2c3ccccc3c3ccccc32)cc(C#N)c1C#N ; N#Cc1cc(C#N)c(-n2c3ccccc3c3ccccc32)cc1-c1cc(C#N)c(C#N)c(-n2c3ccccc3c3ccc4c5ccccc5oc4c32)c1 ; N#Cc1cc(-c2c(C#N)cc(C#N)c(-n3c4ccccc4c4ccccc43)c2C#N)cc(-n2c3ccccc3c3ccc4oc5ccccc5c4c32)c1]

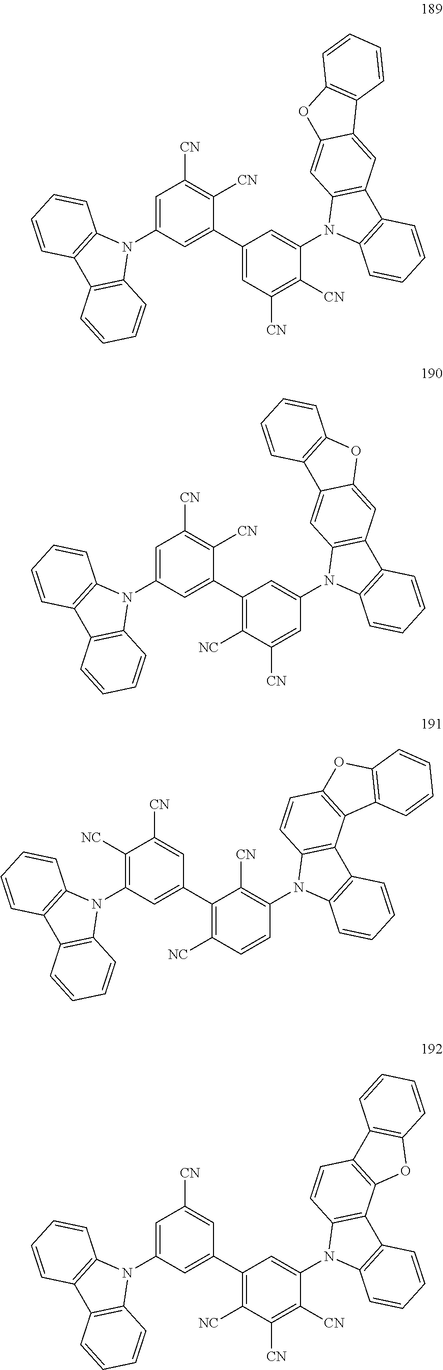 [SMILES: N#Cc1cc(-n2c3ccccc3c3ccccc32)cc(-c2cc(C#N)c(C#N)c(-n3c4ccccc4c4cc5c(cc43)oc3ccccc35)c2)c1C#N ; N#Cc1cc(-n2c3ccccc3c3ccccc32)cc(-c2cc(-n3c4ccccc4c4cc5oc6ccccc6c5cc43)cc(C#N)c2C#N)c1C#N ; N#Cc1cc(-c2c(C#N)ccc(-n3c4ccccc4c4c5c(ccc43)oc3ccccc35)c2C#N)cc(-n2c3ccccc3c3ccccc32)c1C#N ; N#Cc1cc(-c2cc(-n3c4ccccc4c4c5oc6ccccc6c5ccc43)c(C#N)c(C#N)c2C#N)cc(-n2c3ccccc3c3ccccc32)c1]

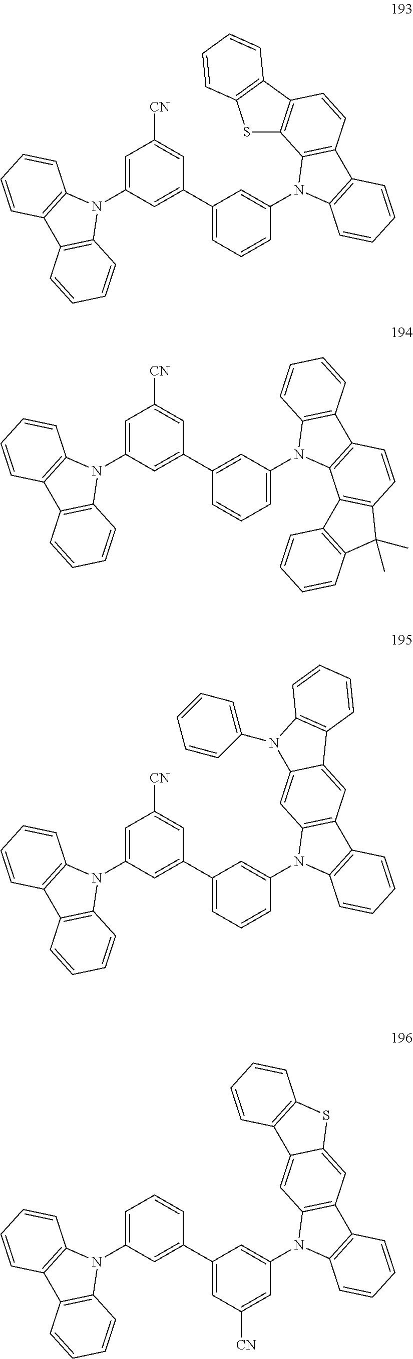 [SMILES: N#Cc1cc(-c2cccc(-n3c4ccccc4c4ccc5c6ccccc6sc5c43)c2)cc(-n2c3ccccc3c3ccccc32)c1 ; CC1(C)c2ccccc2-c2c1ccc1c3ccccc3n(-c3cccc(-c4cc(C#N)cc(-n5c6ccccc6c6ccccc65)c4)c3)c21 ; N#Cc1cc(-c2cccc(-n3c4ccccc4c4cc5c6ccccc6n(-c6ccccc6)c5cc43)c2)cc(-n2c3ccccc3c3ccccc32)c1 ; N#Cc1cc(-c2cccc(-n3c4ccccc4c4ccccc43)c2)cc(-n2c3ccccc3c3cc4sc5ccccc5c4cc32)c1]

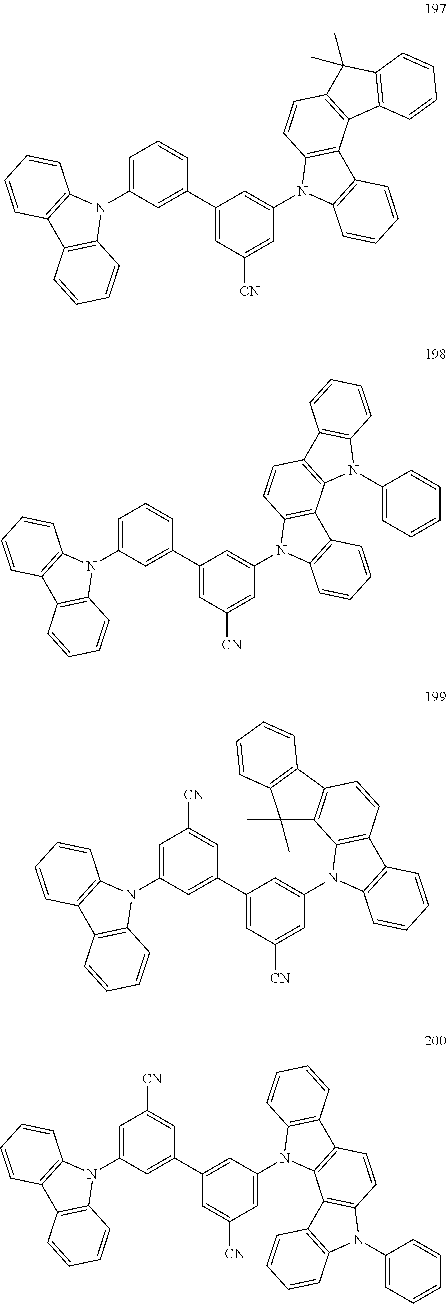 [SMILES: CC1(C)c2ccccc2-c2c1ccc1c2c2ccccc2n1-c1cc(C#N)cc(-c2cccc(-n3c4ccccc4c4ccccc43)c2)c1 ; N#Cc1cc(-c2cccc(-n3c4ccccc4c4ccccc43)c2)cc(-n2c3ccccc3c3c2ccc2c4ccccc4n(-c4ccccc4)c23)c1 ; CC1(C)c2ccccc2-c2ccc3c4ccccc4n(-c4cc(C#N)cc(-c5cc(C#N)cc(-n6c7ccccc7c7ccccc76)c5)c4)c3c21 ; N#Cc1cc(-c2cc(C#N)cc(-n3c4ccccc4c4ccc5c(c6ccccc6n5-c5ccccc5)c43)c2)cc(-n2c3ccccc3c3ccccc32)c1]

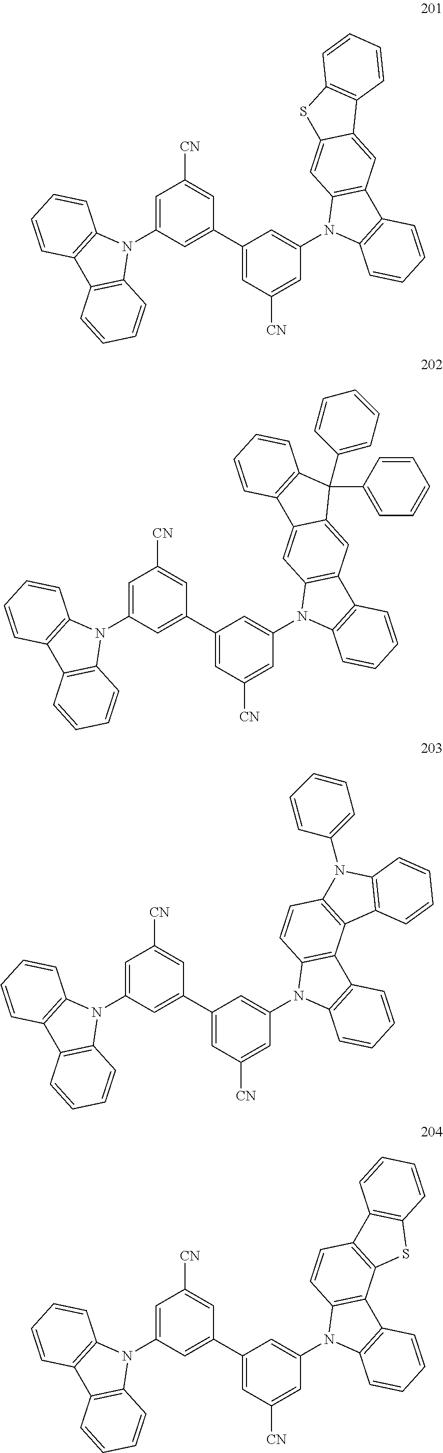 [SMILES: N#Cc1cc(-c2cc(C#N)cc(-n3c4ccccc4c4cc5c(cc43)sc3ccccc35)c2)cc(-n2c3ccccc3c3ccccc32)c1 ; N#Cc1cc(-c2cc(C#N)cc(-n3c4ccccc4c4cc5c(cc43)-c3ccccc3C5(c3ccccc3)c3ccccc3)c2)cc(-n2c3ccccc3c3ccccc32)c1 ; N#Cc1cc(-c2cc(C#N)cc(-n3c4ccccc4c4c5c6ccccc6n(-c6ccccc6)c5ccc43)c2)cc(-n2c3ccccc3c3ccccc32)c1 ; N#Cc1cc(-c2cc(C#N)cc(-n3c4ccccc4c4c5sc6ccccc6c5ccc43)c2)cc(-n2c3ccccc3c3ccccc32)c1]

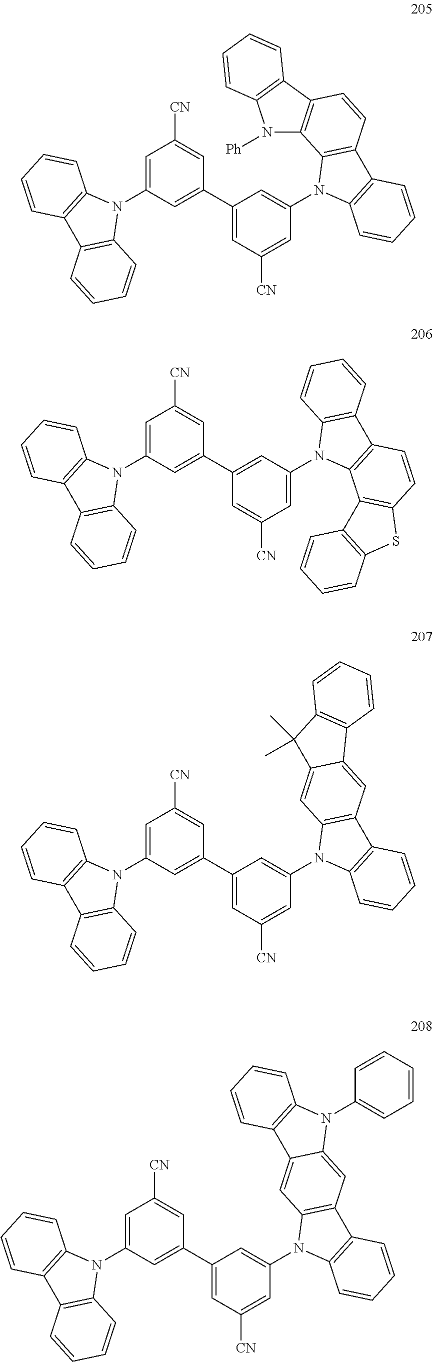 [SMILES: N#Cc1cc(-c2cc(C#N)cc(-n3c4ccccc4c4ccc5c6ccccc6n(-c6ccccc6)c5c43)c2)cc(-n2c3ccccc3c3ccccc32)c1 ; N#Cc1cc(-c2cc(C#N)cc(-n3c4ccccc4c4ccc5sc6ccccc6c5c43)c2)cc(-n2c3ccccc3c3ccccc32)c1 ; CC1(C)c2ccccc2-c2cc3c4ccccc4n(-c4cc(C#N)cc(-c5cc(C#N)cc(-n6c7ccccc7c7ccccc76)c5)c4)c3cc21 ; N#Cc1cc(-c2cc(C#N)cc(-n3c4ccccc4c4cc5c(cc43)c3ccccc3n5-c3ccccc3)c2)cc(-n2c3ccccc3c3ccccc32)c1]

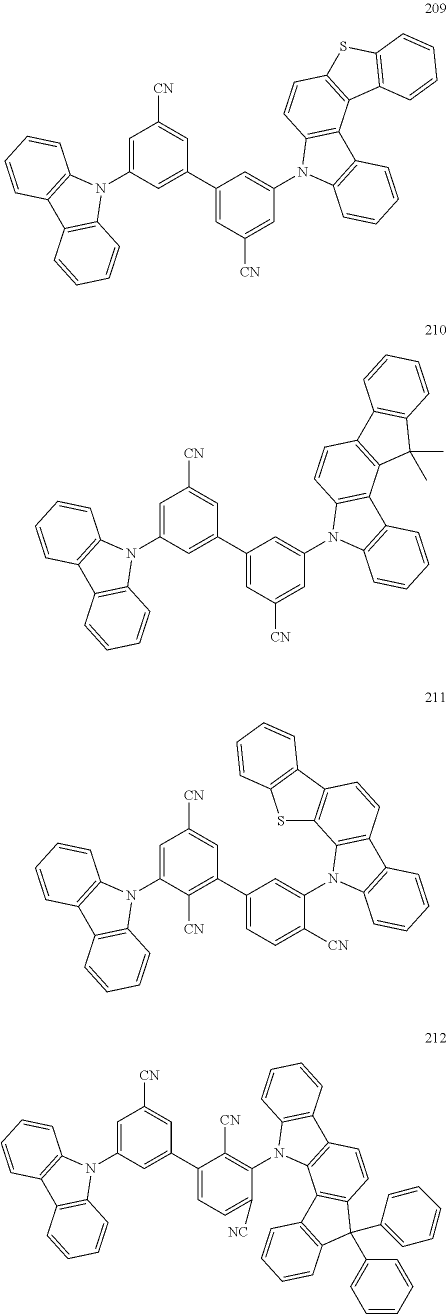 [SMILES: N#Cc1cc(-c2cc(C#N)cc(-n3c4ccccc4c4c5c(ccc43)sc3ccccc35)c2)cc(-n2c3ccccc3c3ccccc32)c1 ; CC1(C)c2ccccc2-c2ccc3c(c21)c1ccccc1n3-c1cc(C#N)cc(-c2cc(C#N)cc(-n3c4ccccc4c4ccccc43)c2)c1 ; N#Cc1cc(-c2ccc(C#N)c(-n3c4ccccc4c4ccc5c6ccccc6sc5c43)c2)c(C#N)c(-n2c3ccccc3c3ccccc32)c1 ; N#Cc1cc(-c2ccc(C#N)c(-n3c4ccccc4c4ccc5c(c43)-c3ccccc3C5(c3ccccc3)c3ccccc3)c2C#N)cc(-n2c3ccccc3c3ccccc32)c1]

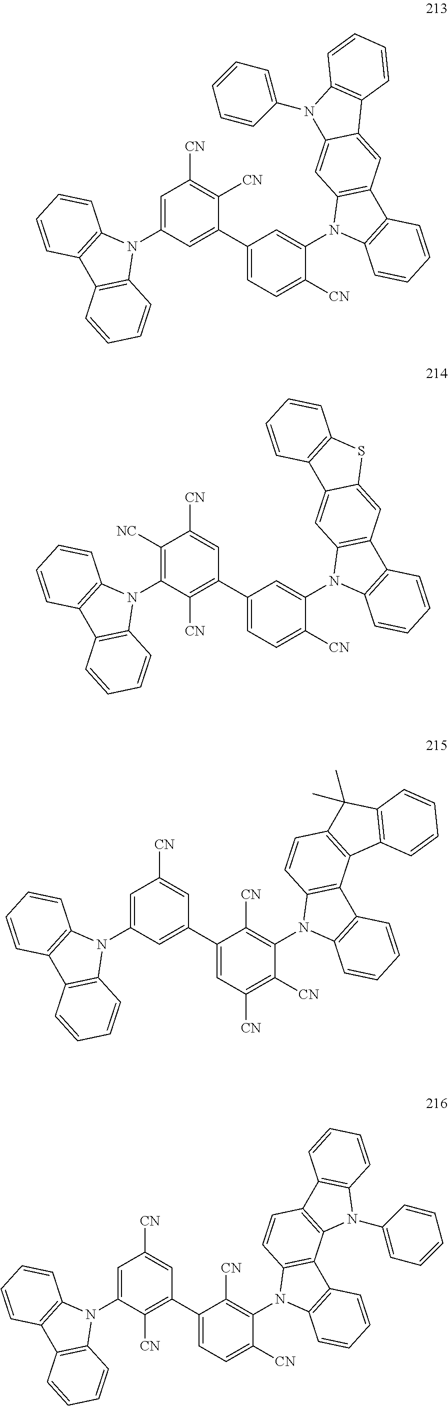 [SMILES: N#Cc1ccc(-c2cc(-n3c4ccccc4c4ccccc43)cc(C#N)c2C#N)cc1-n1c2ccccc2c2cc3c4ccccc4n(-c4ccccc4)c3cc21 ; N#Cc1ccc(-c2cc(C#N)c(C#N)c(-n3c4ccccc4c4ccccc43)c2C#N)cc1-n1c2ccccc2c2cc3sc4ccccc4c3cc21 ; CC1(C)c2ccccc2-c2c1ccc1c2c2ccccc2n1-c1c(C#N)c(C#N)cc(-c2cc(C#N)cc(-n3c4ccccc4c4ccccc43)c2)c1C#N ; N#Cc1cc(-c2ccc(C#N)c(-n3c4ccccc4c4c3ccc3c5ccccc5n(-c5ccccc5)c34)c2C#N)c(C#N)c(-n2c3ccccc3c3ccccc32)c1]

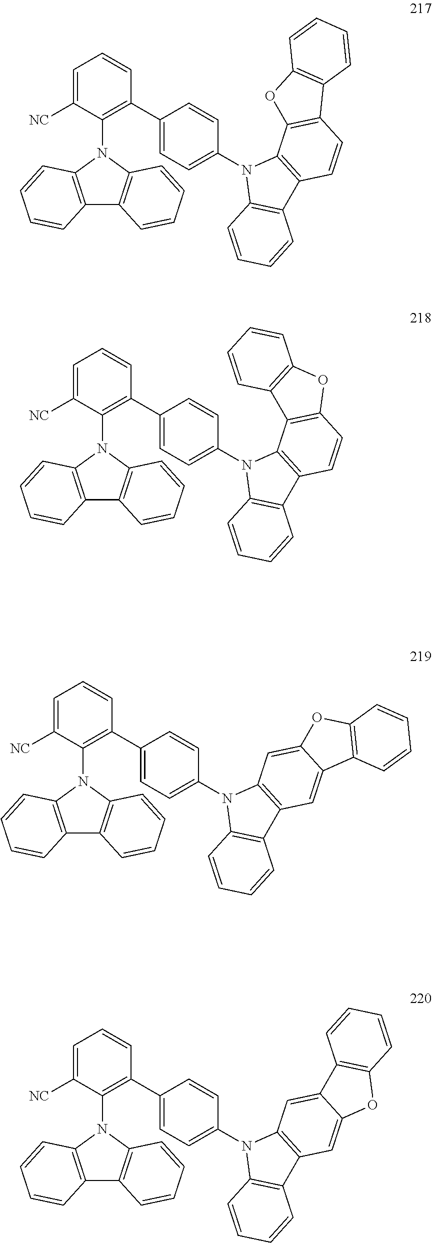 [SMILES: N#Cc1cccc(-c2ccc(-n3c4ccccc4c4ccc5c6ccccc6oc5c43)cc2)c1-n1c2ccccc2c2ccccc21 ; N#Cc1cccc(-c2ccc(-n3c4ccccc4c4ccc5oc6ccccc6c5c43)cc2)c1-n1c2ccccc2c2ccccc21 ; N#Cc1cccc(-c2ccc(-n3c4ccccc4c4cc5c(cc43)oc3ccccc35)cc2)c1-n1c2ccccc2c2ccccc21 ; N#Cc1cccc(-c2ccc(-n3c4ccccc4c4cc5oc6ccccc6c5cc43)cc2)c1-n1c2ccccc2c2ccccc21]

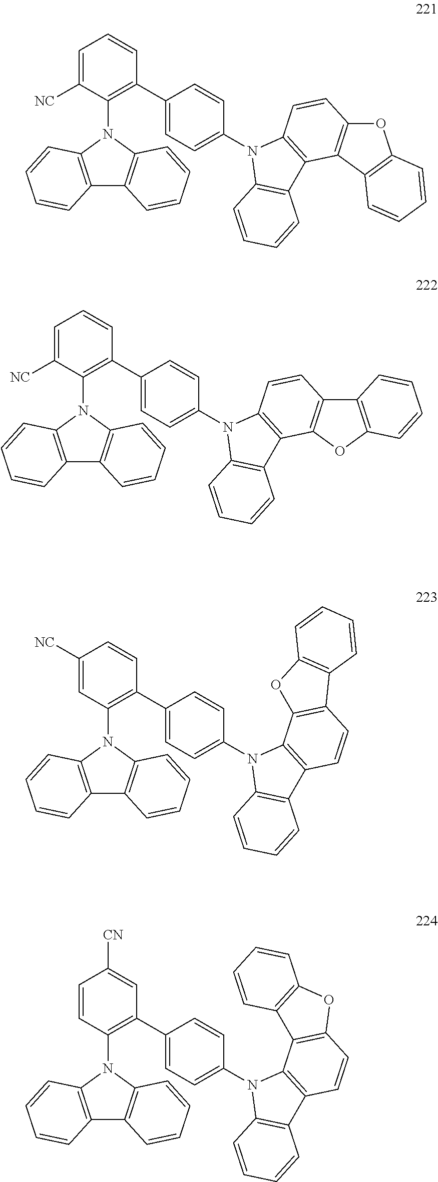 [SMILES: N#Cc1cccc(-c2ccc(-n3c4ccccc4c4c5c(ccc43)oc3ccccc35)cc2)c1-n1c2ccccc2c2ccccc21 ; N#Cc1cccc(-c2ccc(-n3c4ccccc4c4c5oc6ccccc6c5ccc43)cc2)c1-n1c2ccccc2c2ccccc21 ; N#Cc1ccc(-c2ccc(-n3c4ccccc4c4ccc5c6ccccc6oc5c43)cc2)c(-n2c3ccccc3c3ccccc32)c1 ; N#Cc1ccc(-n2c3ccccc3c3ccccc32)c(-c2ccc(-n3c4ccccc4c4ccc5oc6ccccc6c5c43)cc2)c1]

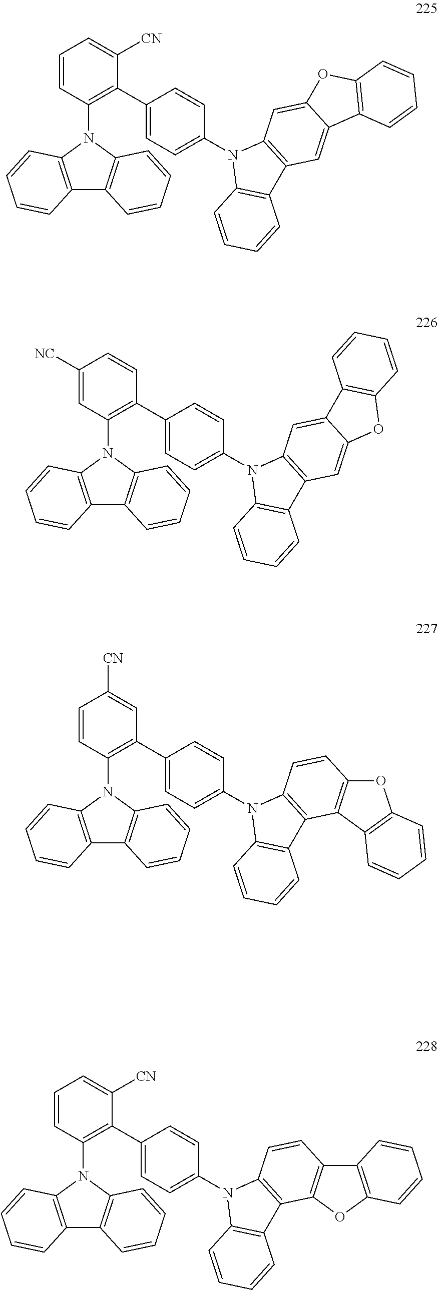 [SMILES: N#Cc1cccc(-n2c3ccccc3c3ccccc32)c1-c1ccc(-n2c3ccccc3c3cc4c(cc32)oc2ccccc24)cc1 ; N#Cc1ccc(-c2ccc(-n3c4ccccc4c4cc5oc6ccccc6c5cc43)cc2)c(-n2c3ccccc3c3ccccc32)c1 ; N#Cc1ccc(-n2c3ccccc3c3ccccc32)c(-c2ccc(-n3c4ccccc4c4c5c(ccc43)oc3ccccc35)cc2)c1 ; N#Cc1cccc(-n2c3ccccc3c3ccccc32)c1-c1ccc(-n2c3ccccc3c3c4oc5ccccc5c4ccc32)cc1]

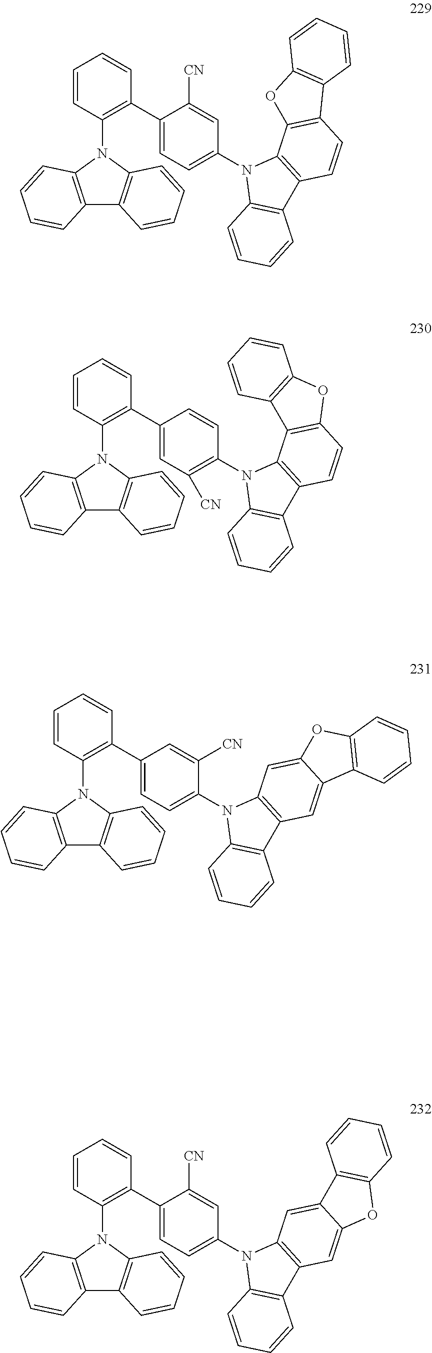 [SMILES: N#Cc1cc(-n2c3ccccc3c3ccc4c5ccccc5oc4c32)ccc1-c1ccccc1-n1c2ccccc2c2ccccc21 ; N#Cc1cc(-c2ccccc2-n2c3ccccc3c3ccccc32)ccc1-n1c2ccccc2c2ccc3oc4ccccc4c3c21 ; N#Cc1cc(-c2ccccc2-n2c3ccccc3c3ccccc32)ccc1-n1c2ccccc2c2cc3c(cc21)oc1ccccc13 ; N#Cc1cc(-n2c3ccccc3c3cc4oc5ccccc5c4cc32)ccc1-c1ccccc1-n1c2ccccc2c2ccccc21]

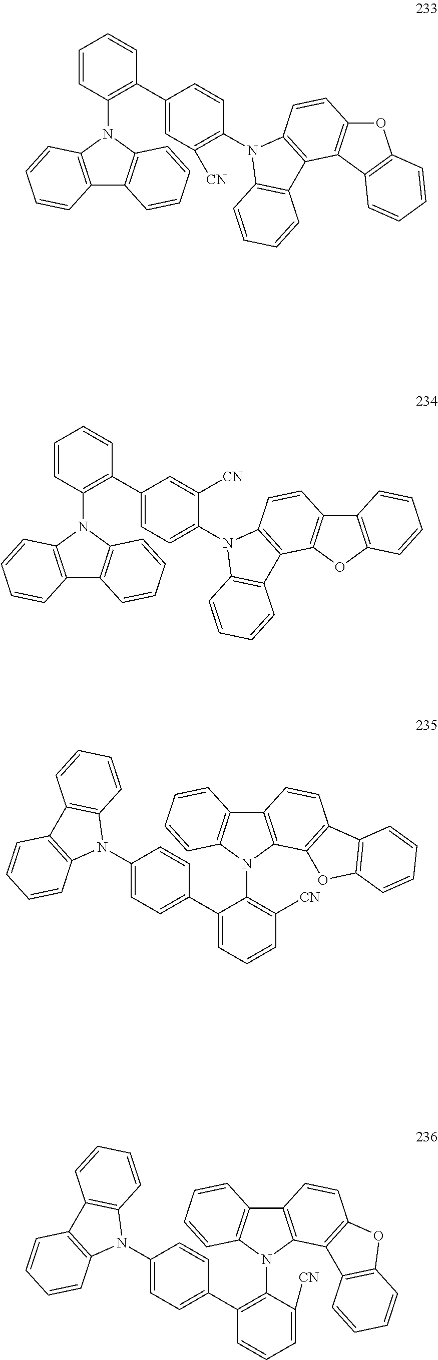 [SMILES: N#Cc1cc(-c2ccccc2-n2c3ccccc3c3ccccc32)ccc1-n1c2ccccc2c2c3c(ccc21)oc1ccccc13 ; N#Cc1cc(-c2ccccc2-n2c3ccccc3c3ccccc32)ccc1-n1c2ccccc2c2c3oc4ccccc4c3ccc21 ; N#Cc1cccc(-c2ccc(-n3c4ccccc4c4ccccc43)cc2)c1-n1c2ccccc2c2ccc3c4ccccc4oc3c21 ; N#Cc1cccc(-c2ccc(-n3c4ccccc4c4ccccc43)cc2)c1-n1c2ccccc2c2ccc3oc4ccccc4c3c21]

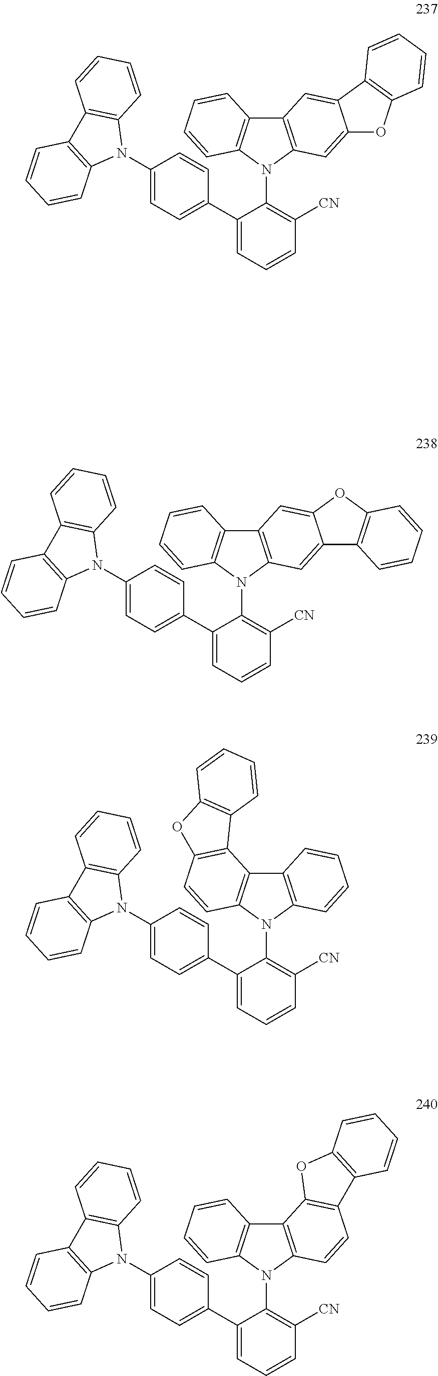 [SMILES: N#Cc1cccc(-c2ccc(-n3c4ccccc4c4ccccc43)cc2)c1-n1c2ccccc2c2cc3c(cc21)oc1ccccc13 ; N#Cc1cccc(-c2ccc(-n3c4ccccc4c4ccccc43)cc2)c1-n1c2ccccc2c2cc3oc4ccccc4c3cc21 ; N#Cc1cccc(-c2ccc(-n3c4ccccc4c4ccccc43)cc2)c1-n1c2ccccc2c2c3c(ccc21)oc1ccccc13 ; N#Cc1cccc(-c2ccc(-n3c4ccccc4c4ccccc43)cc2)c1-n1c2ccccc2c2c3oc4ccccc4c3ccc21]

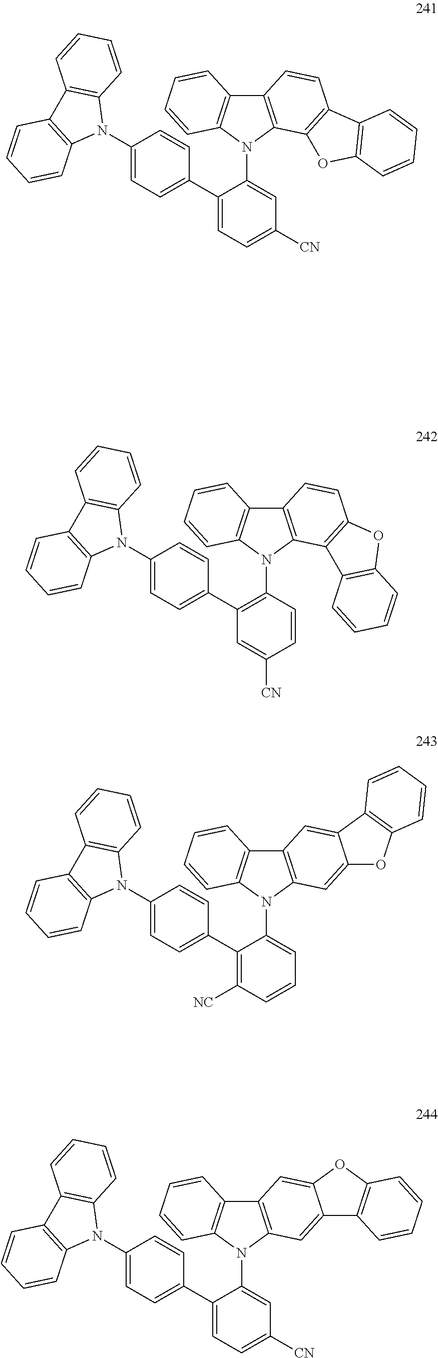 [SMILES: N#Cc1ccc(-c2ccc(-n3c4ccccc4c4ccccc43)cc2)c(-n2c3ccccc3c3ccc4c5ccccc5oc4c32)c1 ; N#Cc1ccc(-n2c3ccccc3c3ccc4oc5ccccc5c4c32)c(-c2ccc(-n3c4ccccc4c4ccccc43)cc2)c1 ; N#Cc1cccc(-n2c3ccccc3c3cc4c(cc32)oc2ccccc24)c1-c1ccc(-n2c3ccccc3c3ccccc32)cc1 ; N#Cc1ccc(-c2ccc(-n3c4ccccc4c4ccccc43)cc2)c(-n2c3ccccc3c3cc4oc5ccccc5c4cc32)c1]

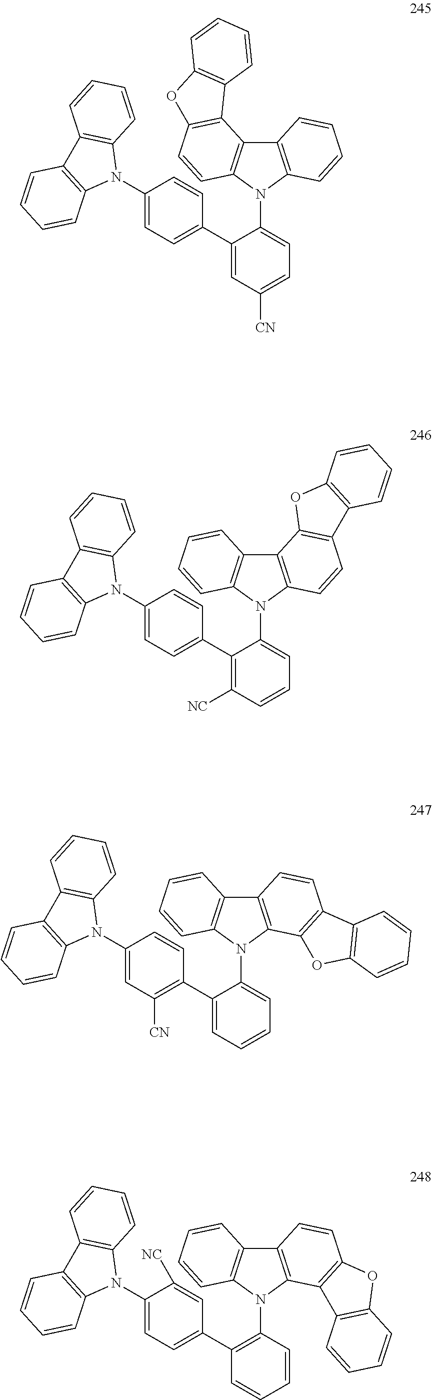 [SMILES: N#Cc1ccc(-n2c3ccccc3c3c4c(ccc32)oc2ccccc24)c(-c2ccc(-n3c4ccccc4c4ccccc43)cc2)c1 ; N#Cc1cccc(-n2c3ccccc3c3c4oc5ccccc5c4ccc32)c1-c1ccc(-n2c3ccccc3c3ccccc32)cc1 ; N#Cc1cc(-n2c3ccccc3c3ccccc32)ccc1-c1ccccc1-n1c2ccccc2c2ccc3c4ccccc4oc3c21 ; N#Cc1cc(-c2ccccc2-n2c3ccccc3c3ccc4oc5ccccc5c4c32)ccc1-n1c2ccccc2c2ccccc21]

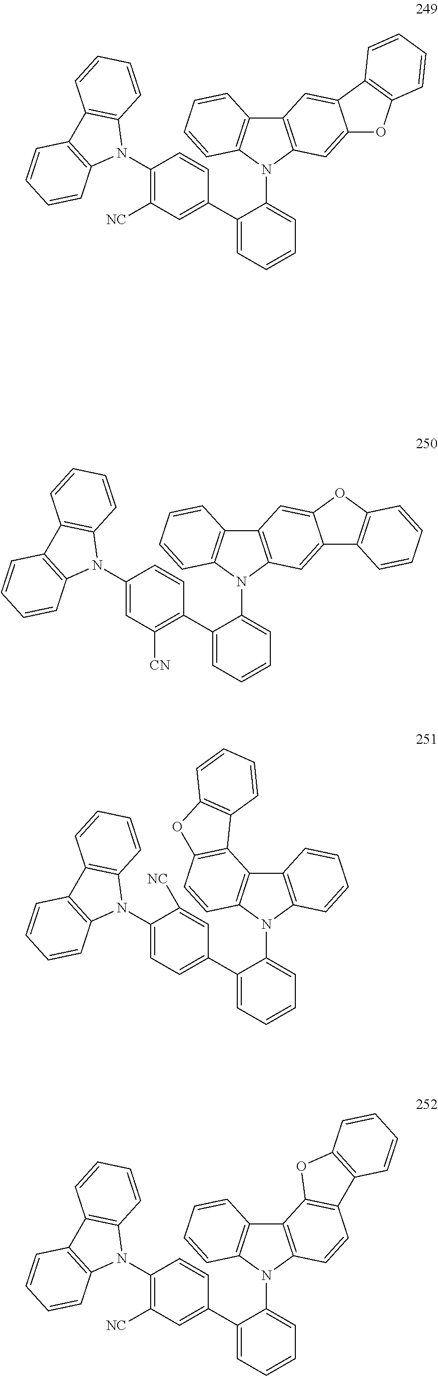 [SMILES: N#Cc1cc(-c2ccccc2-n2c3ccccc3c3cc4c(cc32)oc2ccccc24)ccc1-n1c2ccccc2c2ccccc21 ; N#Cc1cc(-n2c3ccccc3c3ccccc32)ccc1-c1ccccc1-n1c2ccccc2c2cc3oc4ccccc4c3cc21 ; N#Cc1cc(-c2ccccc2-n2c3ccccc3c3c4c(ccc32)oc2ccccc24)ccc1-n1c2ccccc2c2ccccc21 ; N#Cc1cc(-c2ccccc2-n2c3ccccc3c3c4oc5ccccc5c4ccc32)ccc1-n1c2ccccc2c2ccccc21]

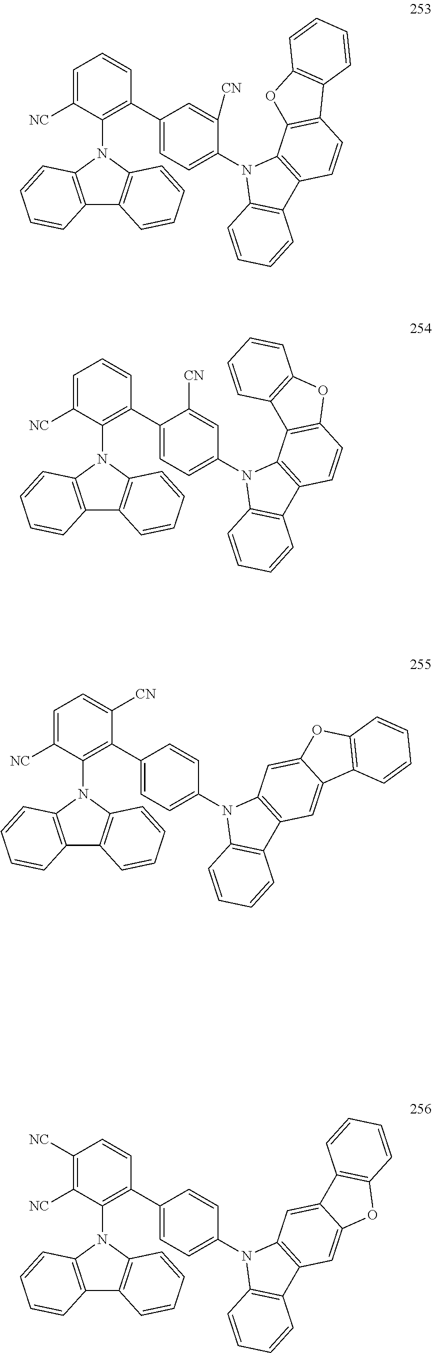 [SMILES: N#Cc1cc(-c2cccc(C#N)c2-n2c3ccccc3c3ccccc32)ccc1-n1c2ccccc2c2ccc3c4ccccc4oc3c21 ; N#Cc1cc(-n2c3ccccc3c3ccc4oc5ccccc5c4c32)ccc1-c1cccc(C#N)c1-n1c2ccccc2c2ccccc21 ; N#Cc1ccc(C#N)c(-n2c3ccccc3c3ccccc32)c1-c1ccc(-n2c3ccccc3c3cc4c(cc32)oc2ccccc24)cc1 ; N#Cc1ccc(-c2ccc(-n3c4ccccc4c4cc5oc6ccccc6c5cc43)cc2)c(-n2c3ccccc3c3ccccc32)c1C#N]

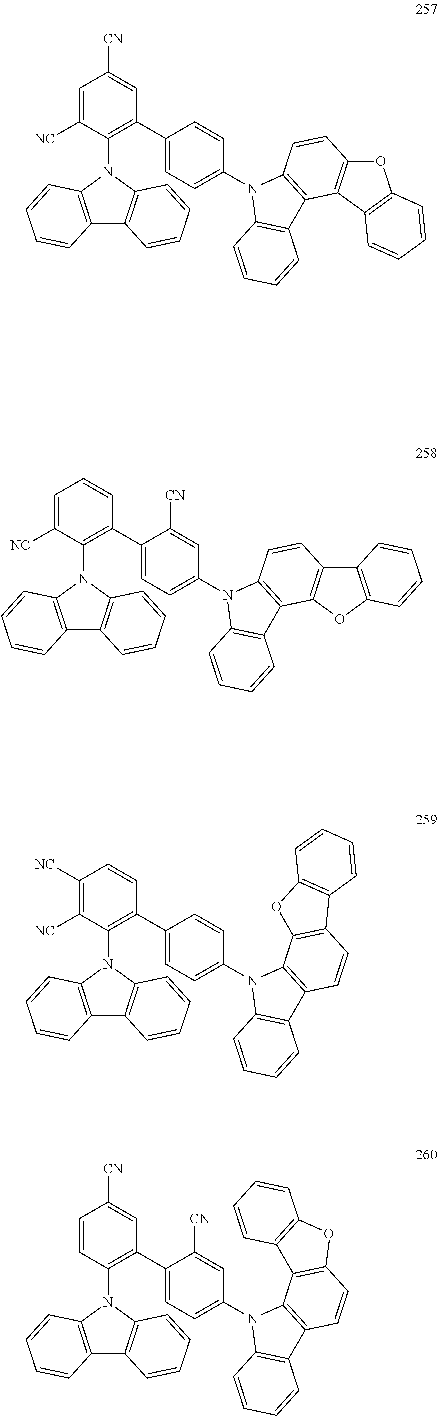 [SMILES: N#Cc1cc(C#N)c(-n2c3ccccc3c3ccccc32)c(-c2ccc(-n3c4ccccc4c4c5c(ccc43)oc3ccccc35)cc2)c1 ; N#Cc1cc(-n2c3ccccc3c3c4oc5ccccc5c4ccc32)ccc1-c1cccc(C#N)c1-n1c2ccccc2c2ccccc21 ; N#Cc1ccc(-c2ccc(-n3c4ccccc4c4ccc5c6ccccc6oc5c43)cc2)c(-n2c3ccccc3c3ccccc32)c1C#N ; N#Cc1ccc(-n2c3ccccc3c3ccccc32)c(-c2ccc(-n3c4ccccc4c4ccc5oc6ccccc6c5c43)cc2C#N)c1]

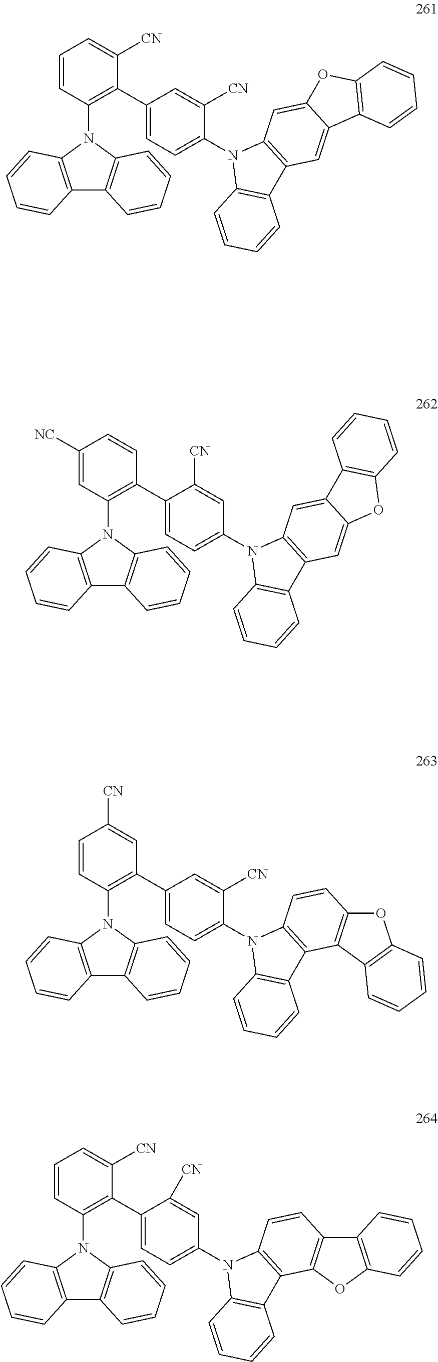 [SMILES: N#Cc1cc(-c2c(C#N)cccc2-n2c3ccccc3c3ccccc32)ccc1-n1c2ccccc2c2cc3c(cc21)oc1ccccc13 ; N#Cc1ccc(-c2ccc(-n3c4ccccc4c4cc5oc6ccccc6c5cc43)cc2C#N)c(-n2c3ccccc3c3ccccc32)c1 ; N#Cc1ccc(-n2c3ccccc3c3ccccc32)c(-c2ccc(-n3c4ccccc4c4c5c(ccc43)oc3ccccc35)c(C#N)c2)c1 ; N#Cc1cc(-n2c3ccccc3c3c4oc5ccccc5c4ccc32)ccc1-c1c(C#N)cccc1-n1c2ccccc2c2ccccc21]

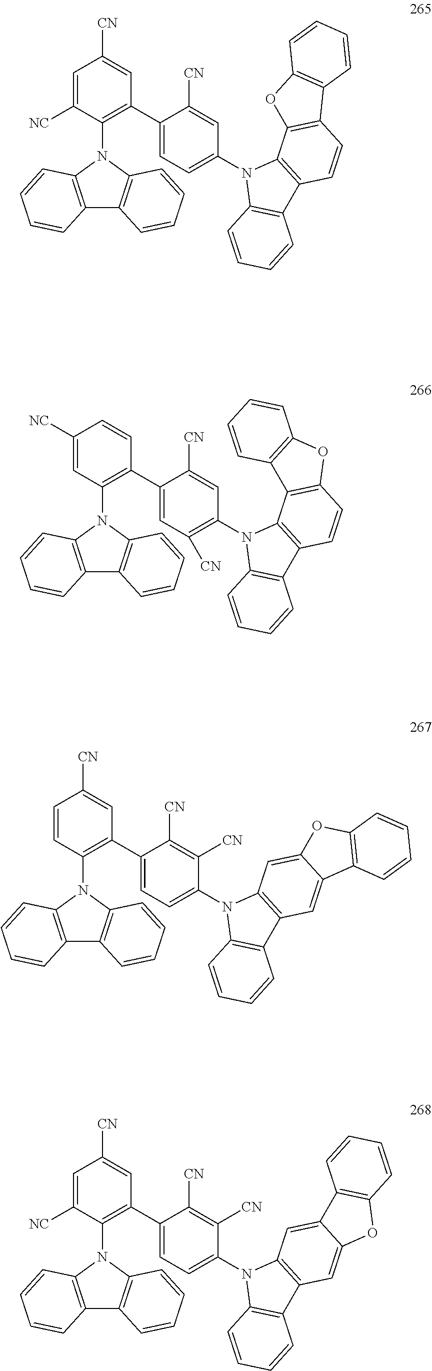 [SMILES: N#Cc1cc(C#N)c(-n2c3ccccc3c3ccccc32)c(-c2ccc(-n3c4ccccc4c4ccc5c6ccccc6oc5c43)cc2C#N)c1 ; N#Cc1ccc(-c2cc(C#N)c(-n3c4ccccc4c4ccc5oc6ccccc6c5c43)cc2C#N)c(-n2c3ccccc3c3ccccc32)c1 ; N#Cc1ccc(-n2c3ccccc3c3ccccc32)c(-c2ccc(-n3c4ccccc4c4cc5c(cc43)oc3ccccc35)c(C#N)c2C#N)c1 ; N#Cc1cc(C#N)c(-n2c3ccccc3c3ccccc32)c(-c2ccc(-n3c4ccccc4c4cc5oc6ccccc6c5cc43)c(C#N)c2C#N)c1]

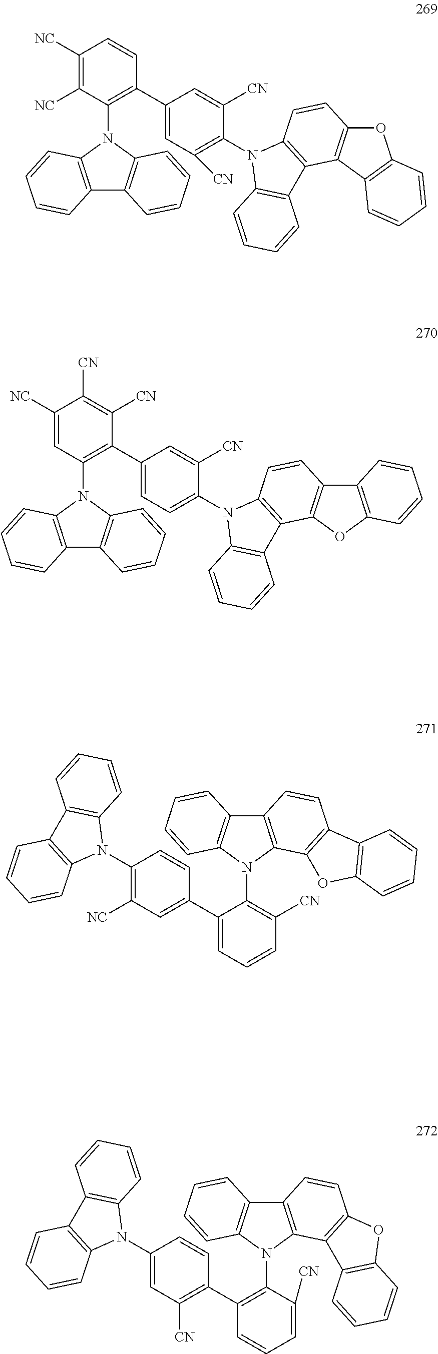 [SMILES: N#Cc1ccc(-c2cc(C#N)c(-n3c4ccccc4c4c5c(ccc43)oc3ccccc35)c(C#N)c2)c(-n2c3ccccc3c3ccccc32)c1C#N ; N#Cc1cc(-c2c(-n3c4ccccc4c4ccccc43)cc(C#N)c(C#N)c2C#N)ccc1-n1c2ccccc2c2c3oc4ccccc4c3ccc21 ; N#Cc1cc(-c2cccc(C#N)c2-n2c3ccccc3c3ccc4c5ccccc5oc4c32)ccc1-n1c2ccccc2c2ccccc21 ; N#Cc1cc(-n2c3ccccc3c3ccccc32)ccc1-c1cccc(C#N)c1-n1c2ccccc2c2ccc3oc4ccccc4c3c21]

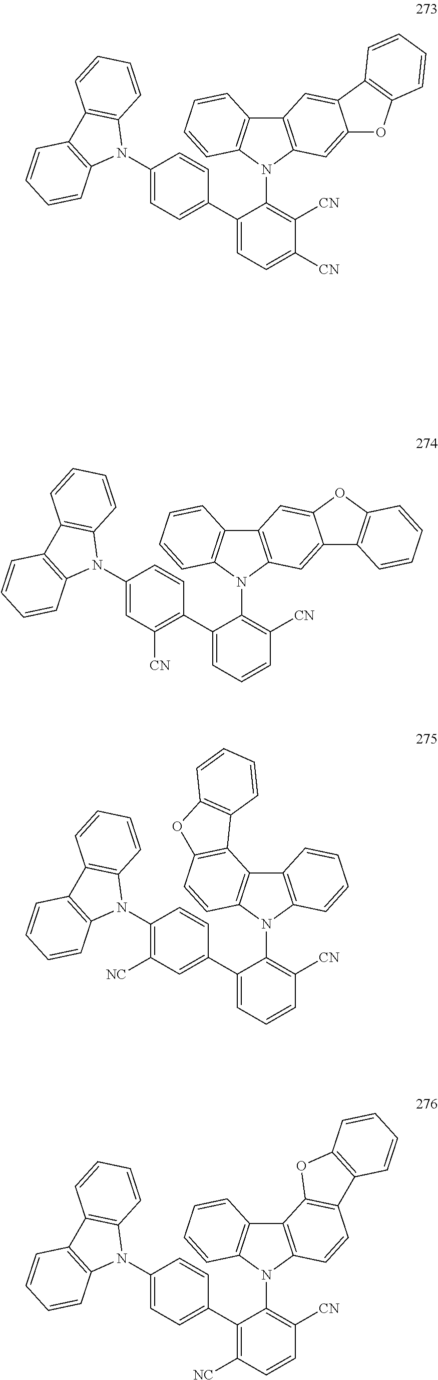 [SMILES: N#Cc1ccc(-c2ccc(-n3c4ccccc4c4ccccc43)cc2)c(-n2c3ccccc3c3cc4c(cc32)oc2ccccc24)c1C#N ; N#Cc1cc(-n2c3ccccc3c3ccccc32)ccc1-c1cccc(C#N)c1-n1c2ccccc2c2cc3oc4ccccc4c3cc21 ; N#Cc1cc(-c2cccc(C#N)c2-n2c3ccccc3c3c4c(ccc32)oc2ccccc24)ccc1-n1c2ccccc2c2ccccc21 ; N#Cc1ccc(C#N)c(-n2c3ccccc3c3c4oc5ccccc5c4ccc32)c1-c1ccc(-n2c3ccccc3c3ccccc32)cc1]

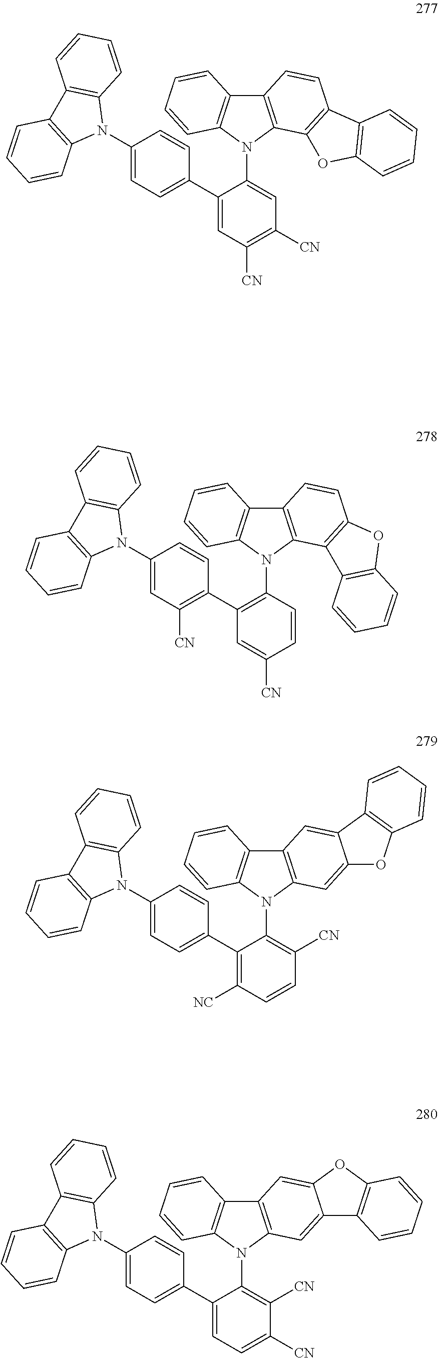 [SMILES: N#Cc1cc(-c2ccc(-n3c4ccccc4c4ccccc43)cc2)c(-n2c3ccccc3c3ccc4c5ccccc5oc4c32)cc1C#N ; N#Cc1ccc(-n2c3ccccc3c3ccc4oc5ccccc5c4c32)c(-c2ccc(-n3c4ccccc4c4ccccc43)cc2C#N)c1 ; N#Cc1ccc(C#N)c(-n2c3ccccc3c3cc4c(cc32)oc2ccccc24)c1-c1ccc(-n2c3ccccc3c3ccccc32)cc1 ; N#Cc1ccc(-c2ccc(-n3c4ccccc4c4ccccc43)cc2)c(-n2c3ccccc3c3cc4oc5ccccc5c4cc32)c1C#N]

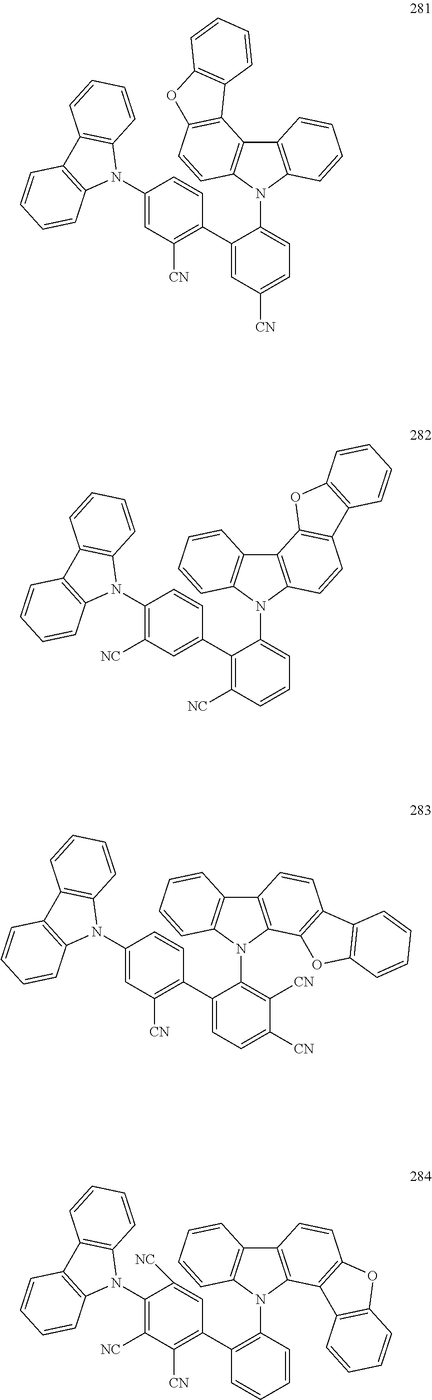 [SMILES: N#Cc1ccc(-n2c3ccccc3c3c4c(ccc32)oc2ccccc24)c(-c2ccc(-n3c4ccccc4c4ccccc43)cc2C#N)c1 ; N#Cc1cc(-c2c(C#N)cccc2-n2c3ccccc3c3c4oc5ccccc5c4ccc32)ccc1-n1c2ccccc2c2ccccc21 ; N#Cc1cc(-n2c3ccccc3c3ccccc32)ccc1-c1ccc(C#N)c(C#N)c1-n1c2ccccc2c2ccc3c4ccccc4oc3c21 ; N#Cc1cc(-c2ccccc2-n2c3ccccc3c3ccc4oc5ccccc5c4c32)c(C#N)c(C#N)c1-n1c2ccccc2c2ccccc21]

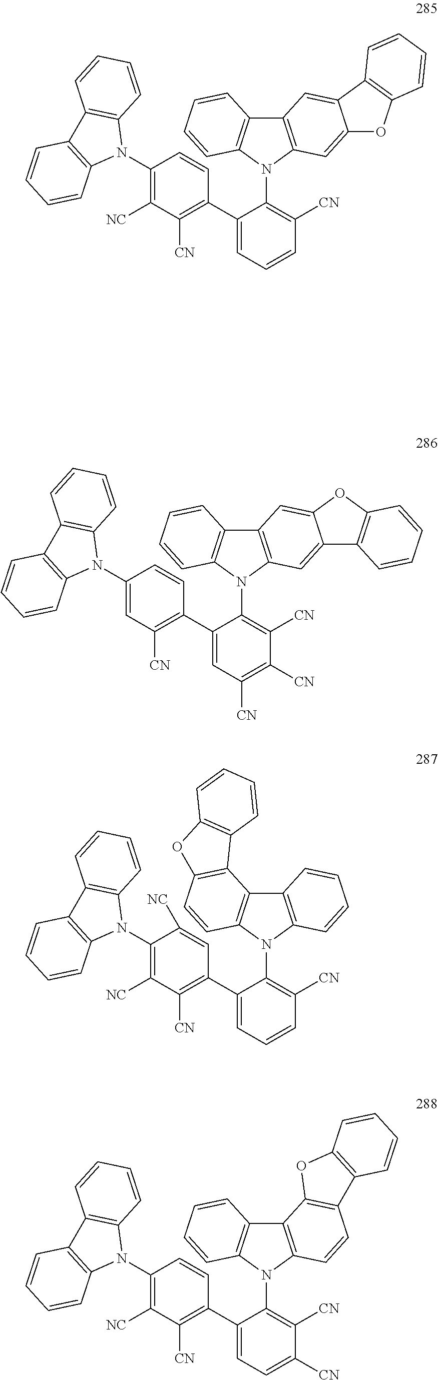 [SMILES: N#Cc1cccc(-c2ccc(-n3c4ccccc4c4ccccc43)c(C#N)c2C#N)c1-n1c2ccccc2c2cc3c(cc21)oc1ccccc13 ; N#Cc1cc(-n2c3ccccc3c3ccccc32)ccc1-c1cc(C#N)c(C#N)c(C#N)c1-n1c2ccccc2c2cc3oc4ccccc4c3cc21 ; N#Cc1cc(-c2cccc(C#N)c2-n2c3ccccc3c3c4c(ccc32)oc2ccccc24)c(C#N)c(C#N)c1-n1c2ccccc2c2ccccc21 ; N#Cc1ccc(-c2ccc(-n3c4ccccc4c4ccccc43)c(C#N)c2C#N)c(-n2c3ccccc3c3c4oc5ccccc5c4ccc32)c1C#N]

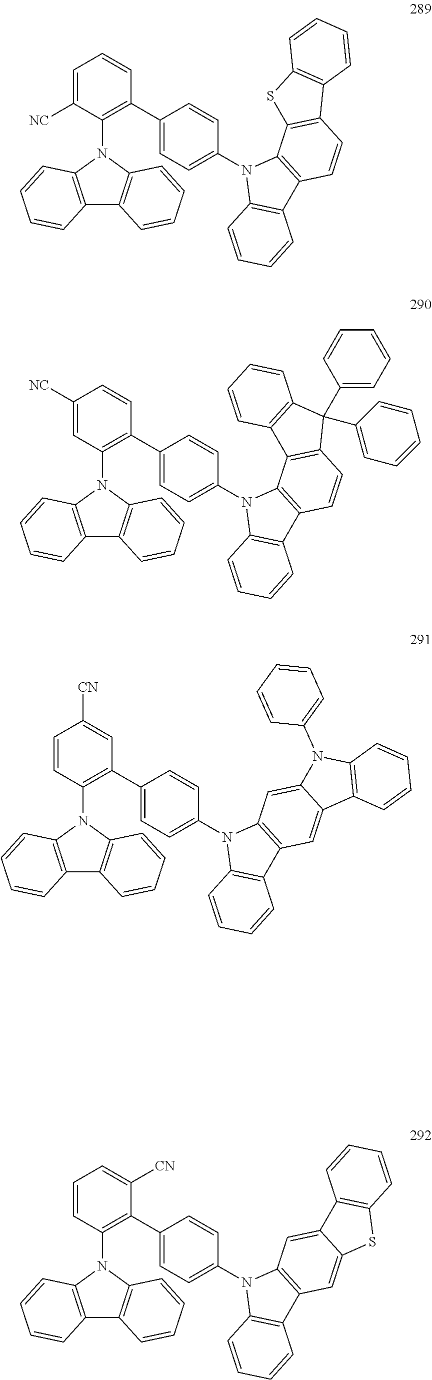 [SMILES: N#Cc1cccc(-c2ccc(-n3c4ccccc4c4ccc5c6ccccc6sc5c43)cc2)c1-n1c2ccccc2c2ccccc21 ; N#Cc1ccc(-c2ccc(-n3c4ccccc4c4ccc5c(c43)-c3ccccc3C5(c3ccccc3)c3ccccc3)cc2)c(-n2c3ccccc3c3ccccc32)c1 ; N#Cc1ccc(-n2c3ccccc3c3ccccc32)c(-c2ccc(-n3c4ccccc4c4cc5c6ccccc6n(-c6ccccc6)c5cc43)cc2)c1 ; N#Cc1cccc(-n2c3ccccc3c3ccccc32)c1-c1ccc(-n2c3ccccc3c3cc4sc5ccccc5c4cc32)cc1]

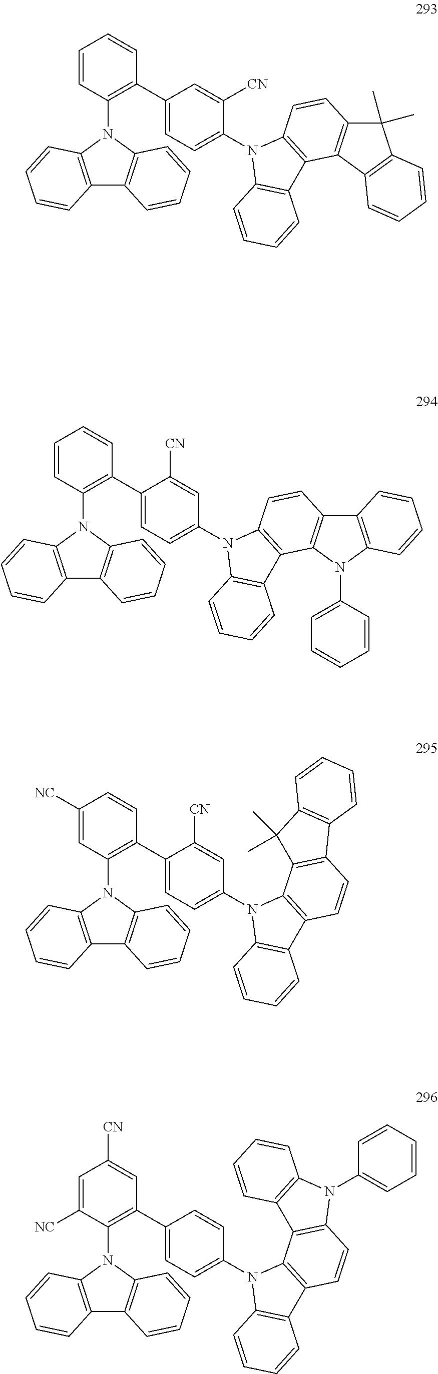 [SMILES: CC1(C)c2ccccc2-c2c1ccc1c2c2ccccc2n1-c1ccc(-c2ccccc2-n2c3ccccc3c3ccccc32)cc1C#N ; N#Cc1cc(-n2c3ccccc3c3c2ccc2c4ccccc4n(-c4ccccc4)c23)ccc1-c1ccccc1-n1c2ccccc2c2ccccc21 ; CC1(C)c2ccccc2-c2ccc3c4ccccc4n(-c4ccc(-c5ccc(C#N)cc5-n5c6ccccc6c6ccccc65)c(C#N)c4)c3c21 ; N#Cc1cc(C#N)c(-n2c3ccccc3c3ccccc32)c(-c2ccc(-n3c4ccccc4c4ccc5c(c6ccccc6n5-c5ccccc5)c43)cc2)c1]

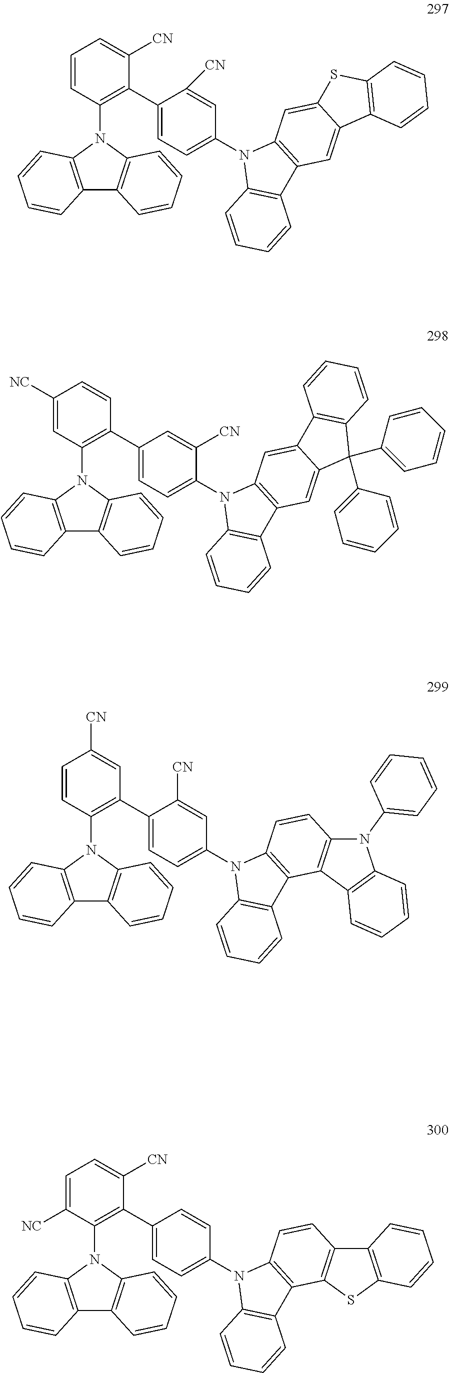 [SMILES: N#Cc1cc(-n2c3ccccc3c3cc4c(cc32)sc2ccccc24)ccc1-c1c(C#N)cccc1-n1c2ccccc2c2ccccc21 ; N#Cc1ccc(-c2ccc(-n3c4ccccc4c4cc5c(cc43)-c3ccccc3C5(c3ccccc3)c3ccccc3)c(C#N)c2)c(-n2c3ccccc3c3ccccc32)c1 ; N#Cc1ccc(-n2c3ccccc3c3ccccc32)c(-c2ccc(-n3c4ccccc4c4c5c6ccccc6n(-c6ccccc6)c5ccc43)cc2C#N)c1 ; N#Cc1ccc(C#N)c(-n2c3ccccc3c3ccccc32)c1-c1ccc(-n2c3ccccc3c3c4sc5ccccc5c4ccc32)cc1]

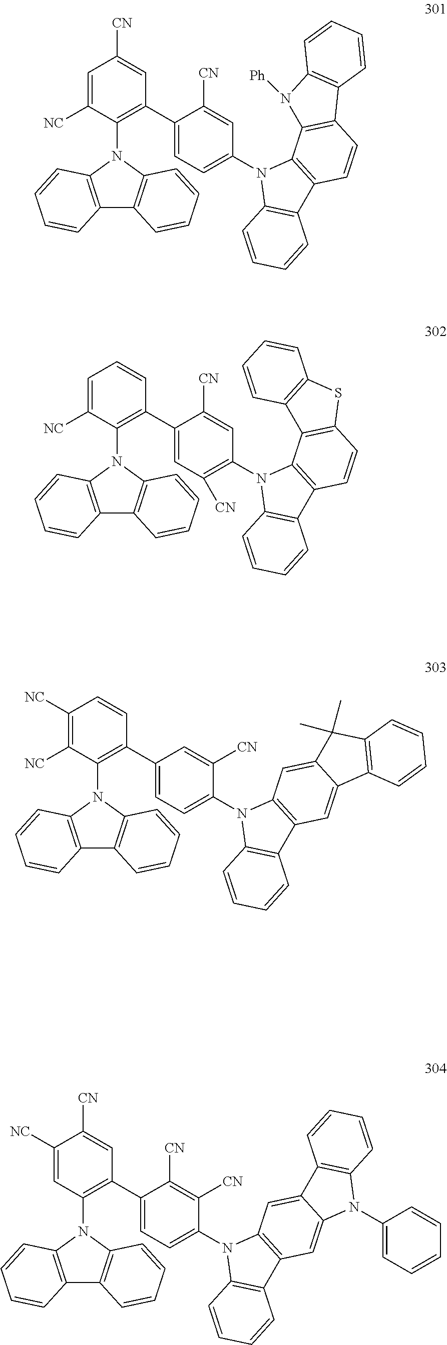 [SMILES: N#Cc1cc(C#N)c(-n2c3ccccc3c3ccccc32)c(-c2ccc(-n3c4ccccc4c4ccc5c6ccccc6n(-c6ccccc6)c5c43)cc2C#N)c1 ; N#Cc1cc(-n2c3ccccc3c3ccc4sc5ccccc5c4c32)c(C#N)cc1-c1cccc(C#N)c1-n1c2ccccc2c2ccccc21 ; CC1(C)c2ccccc2-c2cc3c4ccccc4n(-c4ccc(-c5ccc(C#N)c(C#N)c5-n5c6ccccc6c6ccccc65)cc4C#N)c3cc21 ; N#Cc1cc(-c2ccc(-n3c4ccccc4c4cc5c(cc43)c3ccccc3n5-c3ccccc3)c(C#N)c2C#N)c(-n2c3ccccc3c3ccccc32)cc1C#N]

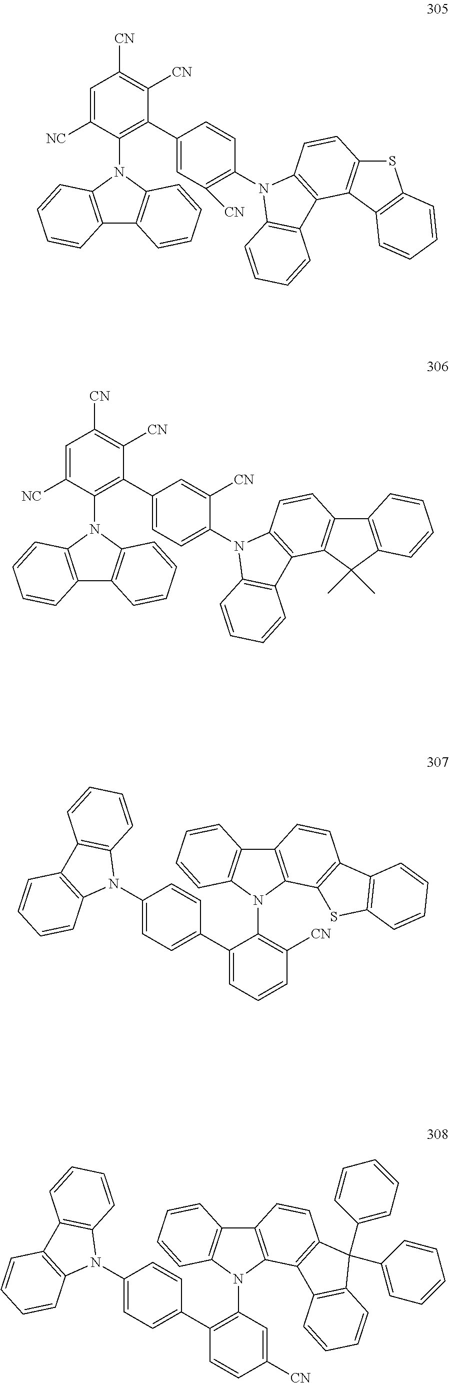 [SMILES: N#Cc1cc(-c2c(C#N)c(C#N)cc(C#N)c2-n2c3ccccc3c3ccccc32)ccc1-n1c2ccccc2c2c3c(ccc21)sc1ccccc13 ; CC1(C)c2ccccc2-c2ccc3c(c21)c1ccccc1n3-c1ccc(-c2c(C#N)c(C#N)cc(C#N)c2-n2c3ccccc3c3ccccc32)cc1C#N ; N#Cc1cccc(-c2ccc(-n3c4ccccc4c4ccccc43)cc2)c1-n1c2ccccc2c2ccc3c4ccccc4sc3c21 ; N#Cc1ccc(-c2ccc(-n3c4ccccc4c4ccccc43)cc2)c(-n2c3ccccc3c3ccc4c(c32)-c2ccccc2C4(c2ccccc2)c2ccccc2)c1]

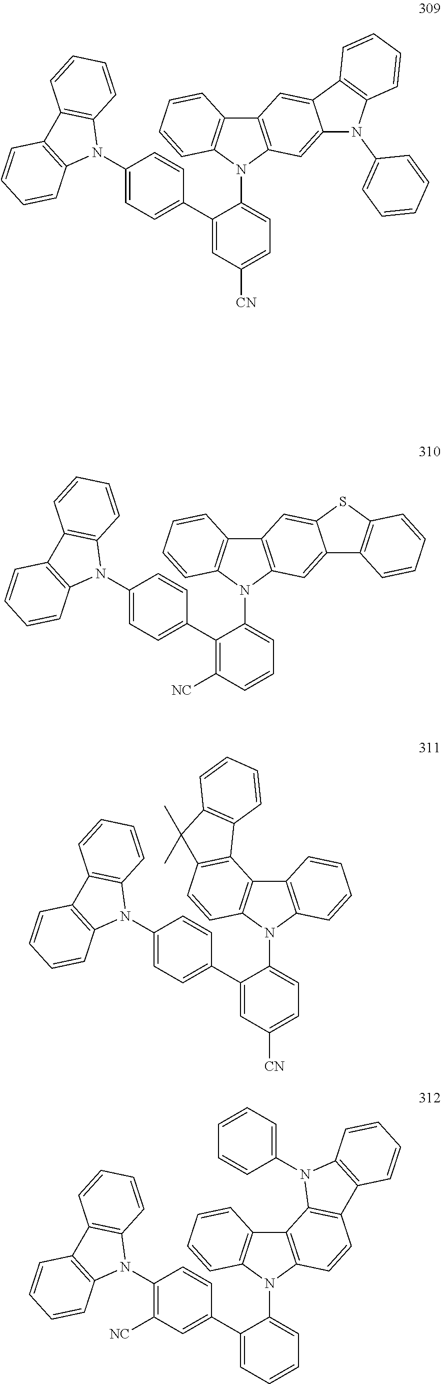 [SMILES: N#Cc1ccc(-n2c3ccccc3c3cc4c5ccccc5n(-c5ccccc5)c4cc32)c(-c2ccc(-n3c4ccccc4c4ccccc43)cc2)c1 ; N#Cc1cccc(-n2c3ccccc3c3cc4sc5ccccc5c4cc32)c1-c1ccc(-n2c3ccccc3c3ccccc32)cc1 ; CC1(C)c2ccccc2-c2c1ccc1c2c2ccccc2n1-c1ccc(C#N)cc1-c1ccc(-n2c3ccccc3c3ccccc32)cc1 ; N#Cc1cc(-c2ccccc2-n2c3ccccc3c3c2ccc2c4ccccc4n(-c4ccccc4)c23)ccc1-n1c2ccccc2c2ccccc21]

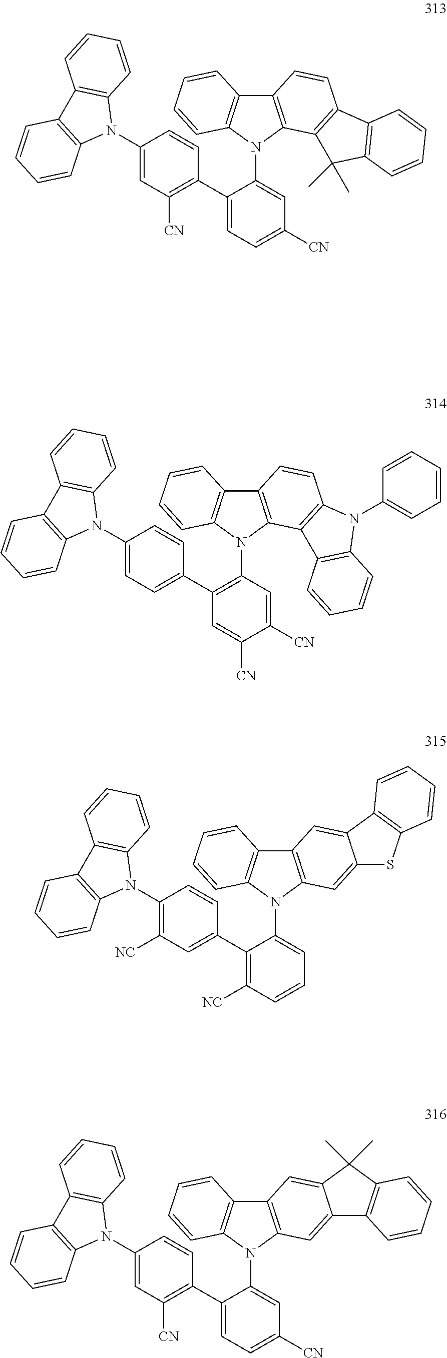 [SMILES: CC1(C)c2ccccc2-c2ccc3c4ccccc4n(-c4cc(C#N)ccc4-c4ccc(-n5c6ccccc6c6ccccc65)cc4C#N)c3c21 ; N#Cc1cc(-c2ccc(-n3c4ccccc4c4ccccc43)cc2)c(-n2c3ccccc3c3ccc4c(c5ccccc5n4-c4ccccc4)c32)cc1C#N ; N#Cc1cc(-c2c(C#N)cccc2-n2c3ccccc3c3cc4c(cc32)sc2ccccc24)ccc1-n1c2ccccc2c2ccccc21 ; CC1(C)c2ccccc2-c2cc3c(cc21)c1ccccc1n3-c1cc(C#N)ccc1-c1ccc(-n2c3ccccc3c3ccccc32)cc1C#N]

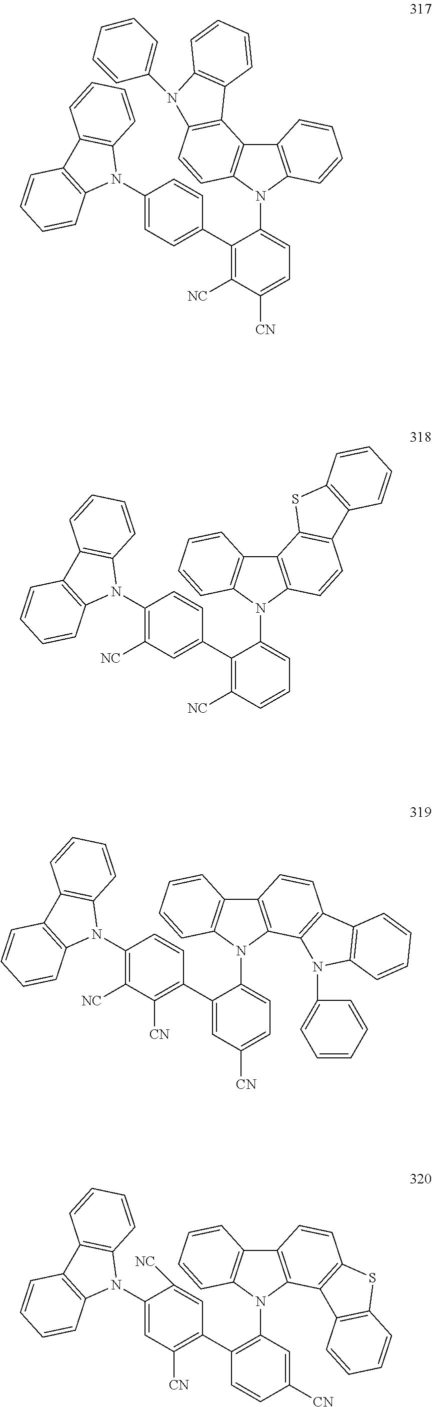 [SMILES: N#Cc1ccc(-n2c3ccccc3c3c4c5ccccc5n(-c5ccccc5)c4ccc32)c(-c2ccc(-n3c4ccccc4c4ccccc43)cc2)c1C#N ; N#Cc1cc(-c2c(C#N)cccc2-n2c3ccccc3c3c4sc5ccccc5c4ccc32)ccc1-n1c2ccccc2c2ccccc21 ; N#Cc1ccc(-n2c3ccccc3c3ccc4c5ccccc5n(-c5ccccc5)c4c32)c(-c2ccc(-n3c4ccccc4c4ccccc43)c(C#N)c2C#N)c1 ; N#Cc1ccc(-c2cc(C#N)c(-n3c4ccccc4c4ccccc43)cc2C#N)c(-n2c3ccccc3c3ccc4sc5ccccc5c4c32)c1]

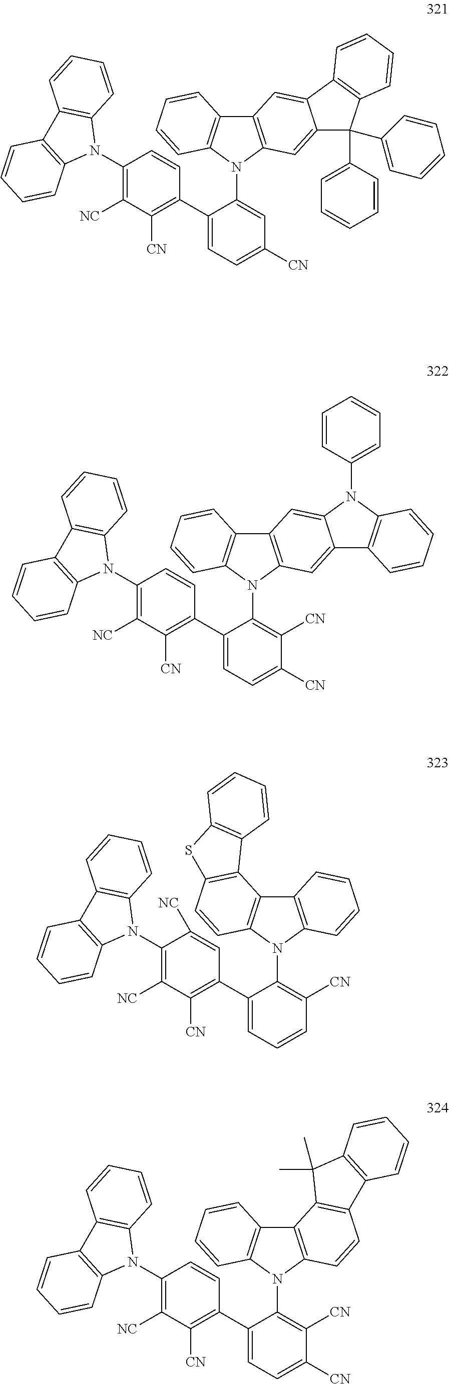 [SMILES: N#Cc1ccc(-c2ccc(-n3c4ccccc4c4ccccc43)c(C#N)c2C#N)c(-n2c3ccccc3c3cc4c(cc32)C(c2ccccc2)(c2ccccc2)c2ccccc2-4)c1 ; N#Cc1ccc(-c2ccc(-n3c4ccccc4c4ccccc43)c(C#N)c2C#N)c(-n2c3ccccc3c3cc4c(cc32)c2ccccc2n4-c2ccccc2)c1C#N ; N#Cc1cc(-c2cccc(C#N)c2-n2c3ccccc3c3c4c(ccc32)sc2ccccc24)c(C#N)c(C#N)c1-n1c2ccccc2c2ccccc21 ; CC1(C)c2ccccc2-c2ccc3c(c21)c1ccccc1n3-c1c(-c2ccc(-n3c4ccccc4c4ccccc43)c(C#N)c2C#N)ccc(C#N)c1C#N]

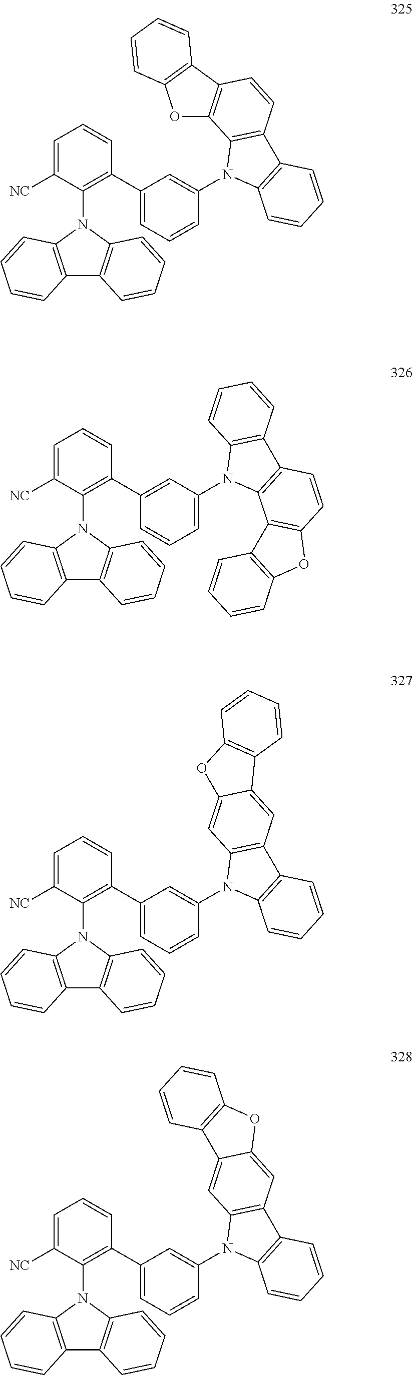 [SMILES: N#Cc1cccc(-c2cccc(-n3c4ccccc4c4ccc5c6ccccc6oc5c43)c2)c1-n1c2ccccc2c2ccccc21 ; N#Cc1cccc(-c2cccc(-n3c4ccccc4c4ccc5oc6ccccc6c5c43)c2)c1-n1c2ccccc2c2ccccc21 ; N#Cc1cccc(-c2cccc(-n3c4ccccc4c4cc5c(cc43)oc3ccccc35)c2)c1-n1c2ccccc2c2ccccc21 ; N#Cc1cccc(-c2cccc(-n3c4ccccc4c4cc5oc6ccccc6c5cc43)c2)c1-n1c2ccccc2c2ccccc21]

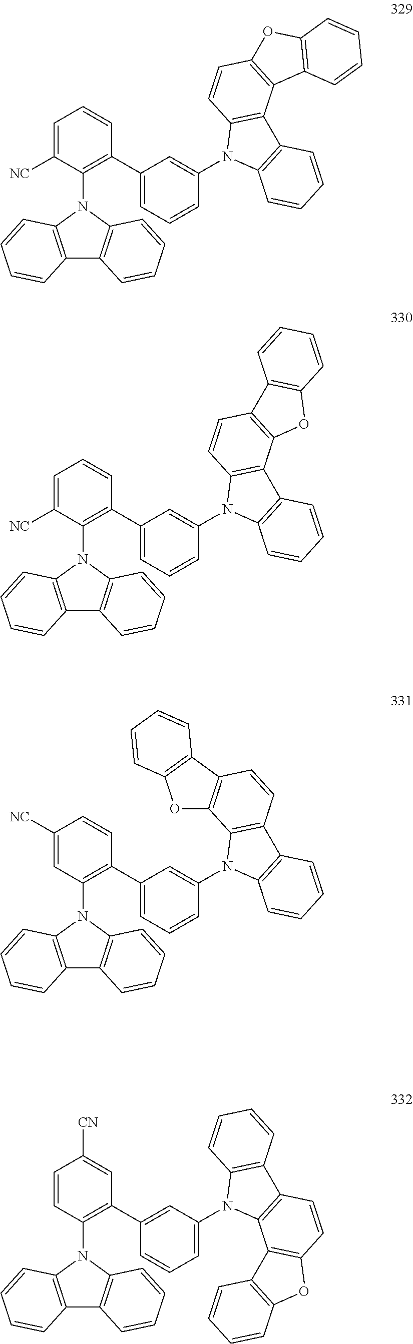 [SMILES: N#Cc1cccc(-c2cccc(-n3c4ccccc4c4c5c(ccc43)oc3ccccc35)c2)c1-n1c2ccccc2c2ccccc21 ; N#Cc1cccc(-c2cccc(-n3c4ccccc4c4c5oc6ccccc6c5ccc43)c2)c1-n1c2ccccc2c2ccccc21 ; N#Cc1ccc(-c2cccc(-n3c4ccccc4c4ccc5c6ccccc6oc5c43)c2)c(-n2c3ccccc3c3ccccc32)c1 ; N#Cc1ccc(-n2c3ccccc3c3ccccc32)c(-c2cccc(-n3c4ccccc4c4ccc5oc6ccccc6c5c43)c2)c1]

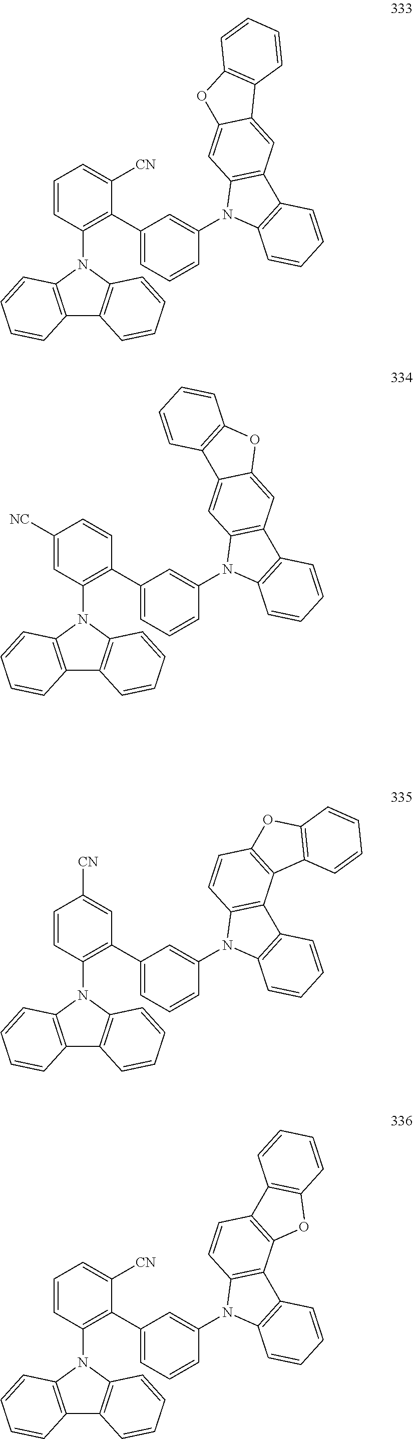 [SMILES: N#Cc1cccc(-n2c3ccccc3c3ccccc32)c1-c1cccc(-n2c3ccccc3c3cc4c(cc32)oc2ccccc24)c1 ; N#Cc1ccc(-c2cccc(-n3c4ccccc4c4cc5oc6ccccc6c5cc43)c2)c(-n2c3ccccc3c3ccccc32)c1 ; N#Cc1ccc(-n2c3ccccc3c3ccccc32)c(-c2cccc(-n3c4ccccc4c4c5c(ccc43)oc3ccccc35)c2)c1 ; N#Cc1cccc(-n2c3ccccc3c3ccccc32)c1-c1cccc(-n2c3ccccc3c3c4oc5ccccc5c4ccc32)c1]

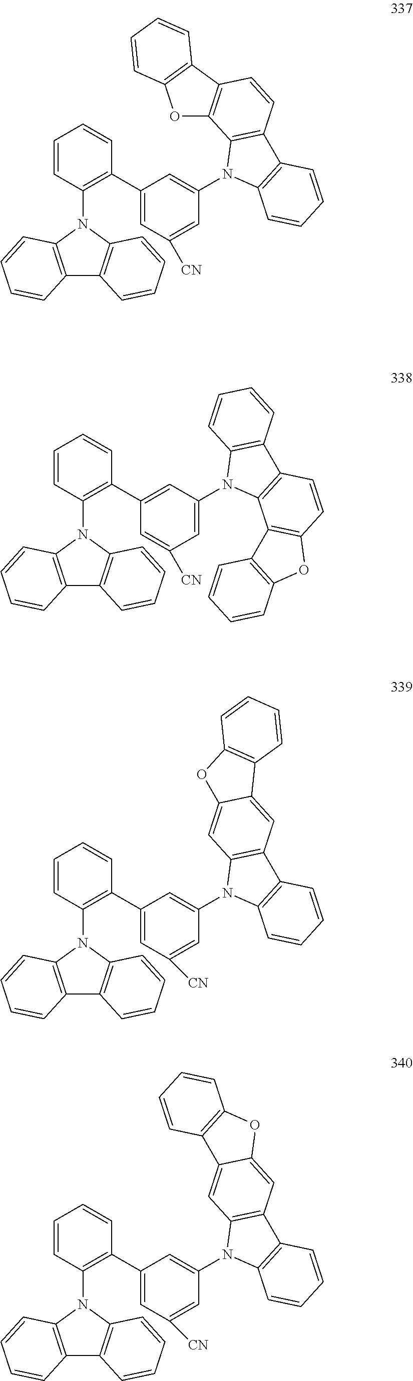 [SMILES: N#Cc1cc(-c2ccccc2-n2c3ccccc3c3ccccc32)cc(-n2c3ccccc3c3ccc4c5ccccc5oc4c32)c1 ; N#Cc1cc(-c2ccccc2-n2c3ccccc3c3ccccc32)cc(-n2c3ccccc3c3ccc4oc5ccccc5c4c32)c1 ; N#Cc1cc(-c2ccccc2-n2c3ccccc3c3ccccc32)cc(-n2c3ccccc3c3cc4c(cc32)oc2ccccc24)c1 ; N#Cc1cc(-c2ccccc2-n2c3ccccc3c3ccccc32)cc(-n2c3ccccc3c3cc4oc5ccccc5c4cc32)c1]

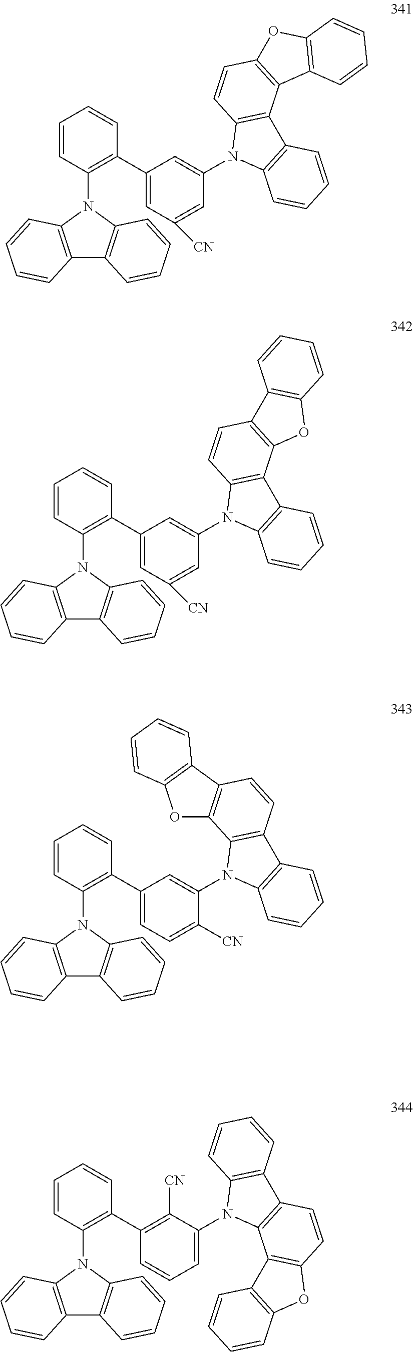 [SMILES: N#Cc1cc(-c2ccccc2-n2c3ccccc3c3ccccc32)cc(-n2c3ccccc3c3c4c(ccc32)oc2ccccc24)c1 ; N#Cc1cc(-c2ccccc2-n2c3ccccc3c3ccccc32)cc(-n2c3ccccc3c3c4oc5ccccc5c4ccc32)c1 ; N#Cc1ccc(-c2ccccc2-n2c3ccccc3c3ccccc32)cc1-n1c2ccccc2c2ccc3c4ccccc4oc3c21 ; N#Cc1c(-c2ccccc2-n2c3ccccc3c3ccccc32)cccc1-n1c2ccccc2c2ccc3oc4ccccc4c3c21]

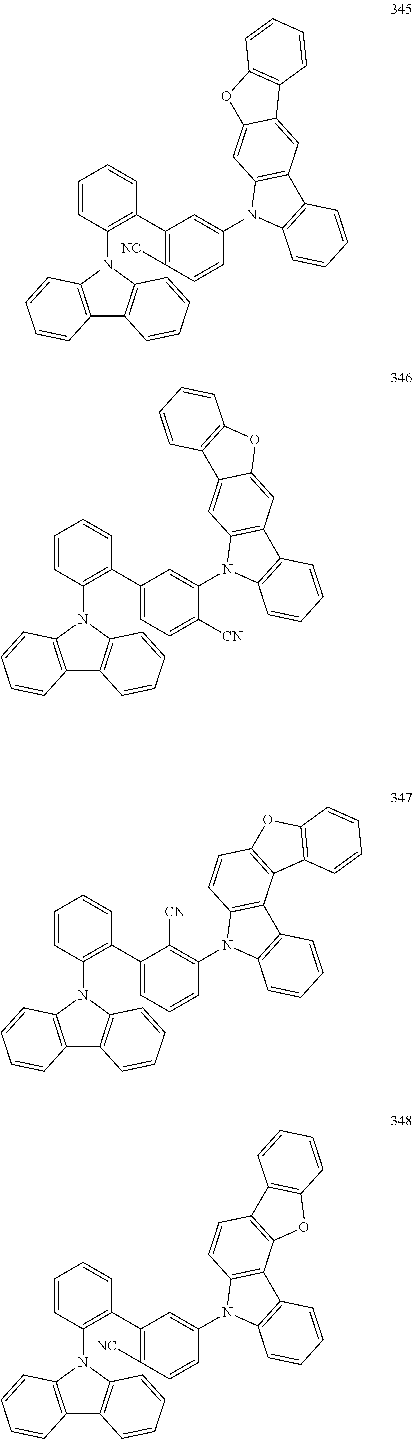 [SMILES: N#Cc1ccc(-n2c3ccccc3c3cc4c(cc32)oc2ccccc24)cc1-c1ccccc1-n1c2ccccc2c2ccccc21 ; N#Cc1ccc(-c2ccccc2-n2c3ccccc3c3ccccc32)cc1-n1c2ccccc2c2cc3oc4ccccc4c3cc21 ; N#Cc1c(-c2ccccc2-n2c3ccccc3c3ccccc32)cccc1-n1c2ccccc2c2c3c(ccc21)oc1ccccc13 ; N#Cc1ccc(-n2c3ccccc3c3c4oc5ccccc5c4ccc32)cc1-c1ccccc1-n1c2ccccc2c2ccccc21]

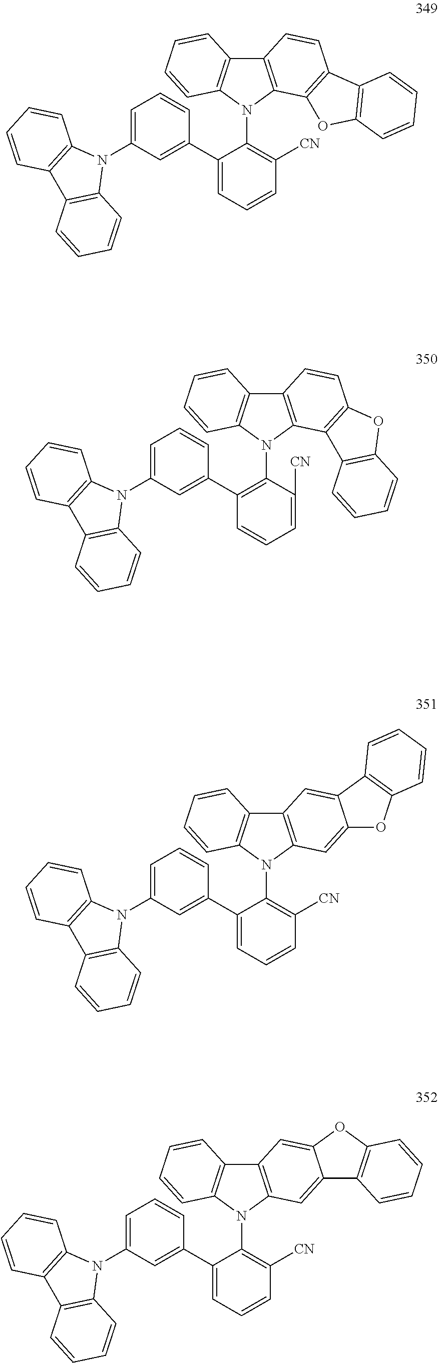 [SMILES: N#Cc1cccc(-c2cccc(-n3c4ccccc4c4ccccc43)c2)c1-n1c2ccccc2c2ccc3c4ccccc4oc3c21 ; N#Cc1cccc(-c2cccc(-n3c4ccccc4c4ccccc43)c2)c1-n1c2ccccc2c2ccc3oc4ccccc4c3c21 ; N#Cc1cccc(-c2cccc(-n3c4ccccc4c4ccccc43)c2)c1-n1c2ccccc2c2cc3c(cc21)oc1ccccc13 ; N#Cc1cccc(-c2cccc(-n3c4ccccc4c4ccccc43)c2)c1-n1c2ccccc2c2cc3oc4ccccc4c3cc21]

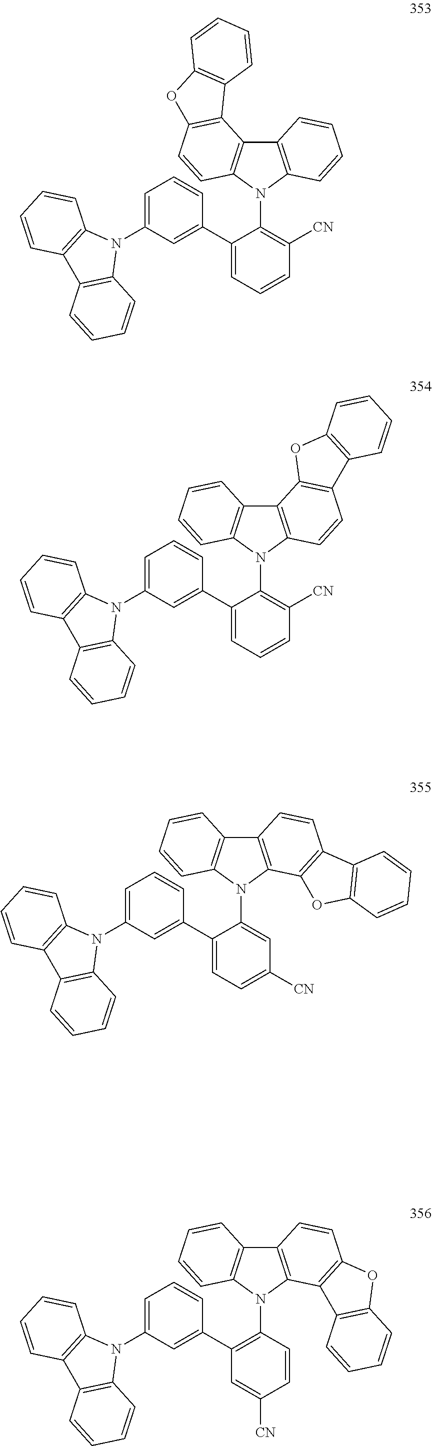 [SMILES: N#Cc1cccc(-c2cccc(-n3c4ccccc4c4ccccc43)c2)c1-n1c2ccccc2c2c3c(ccc21)oc1ccccc13 ; N#Cc1cccc(-c2cccc(-n3c4ccccc4c4ccccc43)c2)c1-n1c2ccccc2c2c3oc4ccccc4c3ccc21 ; N#Cc1ccc(-c2cccc(-n3c4ccccc4c4ccccc43)c2)c(-n2c3ccccc3c3ccc4c5ccccc5oc4c32)c1 ; N#Cc1ccc(-n2c3ccccc3c3ccc4oc5ccccc5c4c32)c(-c2cccc(-n3c4ccccc4c4ccccc43)c2)c1]

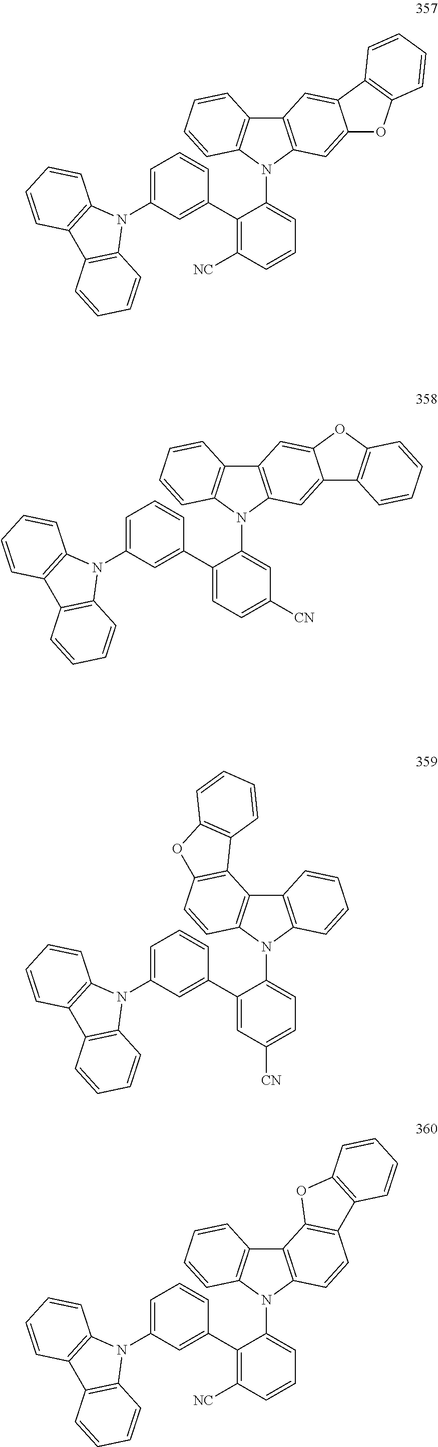 [SMILES: N#Cc1cccc(-n2c3ccccc3c3cc4c(cc32)oc2ccccc24)c1-c1cccc(-n2c3ccccc3c3ccccc32)c1 ; N#Cc1ccc(-c2cccc(-n3c4ccccc4c4ccccc43)c2)c(-n2c3ccccc3c3cc4oc5ccccc5c4cc32)c1 ; N#Cc1ccc(-n2c3ccccc3c3c4c(ccc32)oc2ccccc24)c(-c2cccc(-n3c4ccccc4c4ccccc43)c2)c1 ; N#Cc1cccc(-n2c3ccccc3c3c4oc5ccccc5c4ccc32)c1-c1cccc(-n2c3ccccc3c3ccccc32)c1]

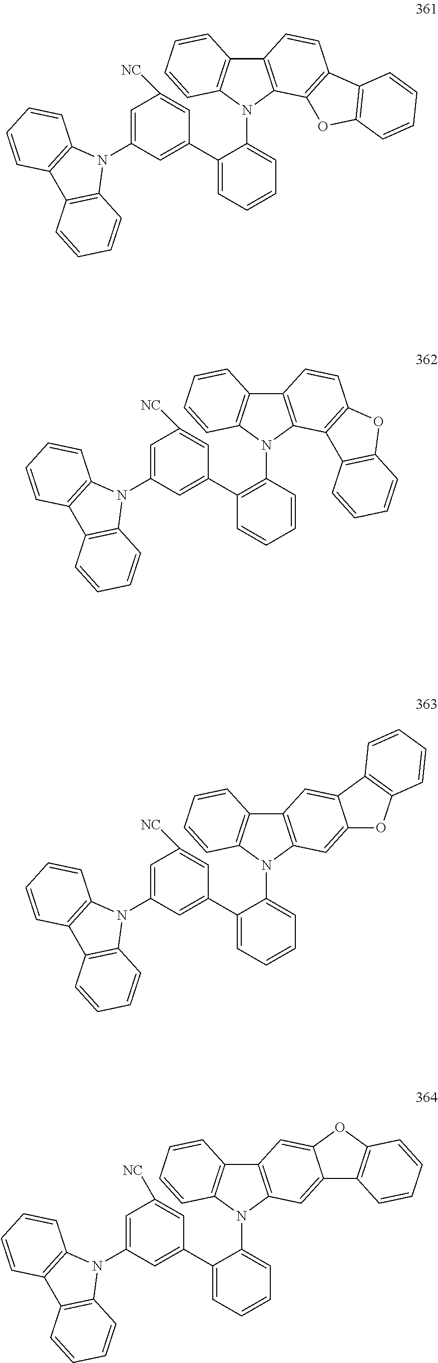 [SMILES: N#Cc1cc(-c2ccccc2-n2c3ccccc3c3ccc4c5ccccc5oc4c32)cc(-n2c3ccccc3c3ccccc32)c1 ; N#Cc1cc(-c2ccccc2-n2c3ccccc3c3ccc4oc5ccccc5c4c32)cc(-n2c3ccccc3c3ccccc32)c1 ; N#Cc1cc(-c2ccccc2-n2c3ccccc3c3cc4c(cc32)oc2ccccc24)cc(-n2c3ccccc3c3ccccc32)c1 ; N#Cc1cc(-c2ccccc2-n2c3ccccc3c3cc4oc5ccccc5c4cc32)cc(-n2c3ccccc3c3ccccc32)c1]

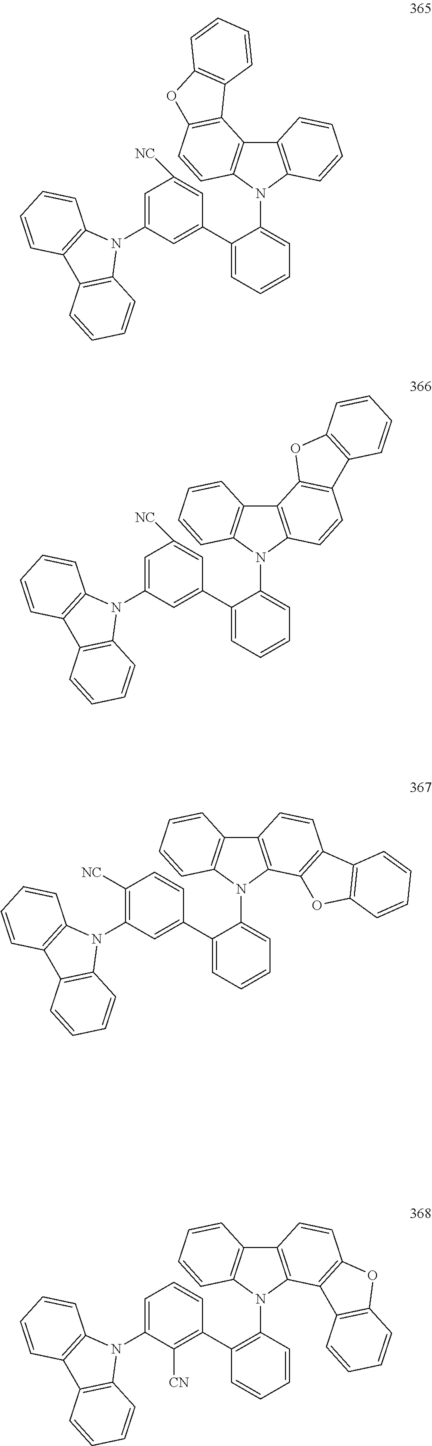 [SMILES: N#Cc1cc(-c2ccccc2-n2c3ccccc3c3c4c(ccc32)oc2ccccc24)cc(-n2c3ccccc3c3ccccc32)c1 ; N#Cc1cc(-c2ccccc2-n2c3ccccc3c3c4oc5ccccc5c4ccc32)cc(-n2c3ccccc3c3ccccc32)c1 ; N#Cc1ccc(-c2ccccc2-n2c3ccccc3c3ccc4c5ccccc5oc4c32)cc1-n1c2ccccc2c2ccccc21 ; N#Cc1c(-c2ccccc2-n2c3ccccc3c3ccc4oc5ccccc5c4c32)cccc1-n1c2ccccc2c2ccccc21]

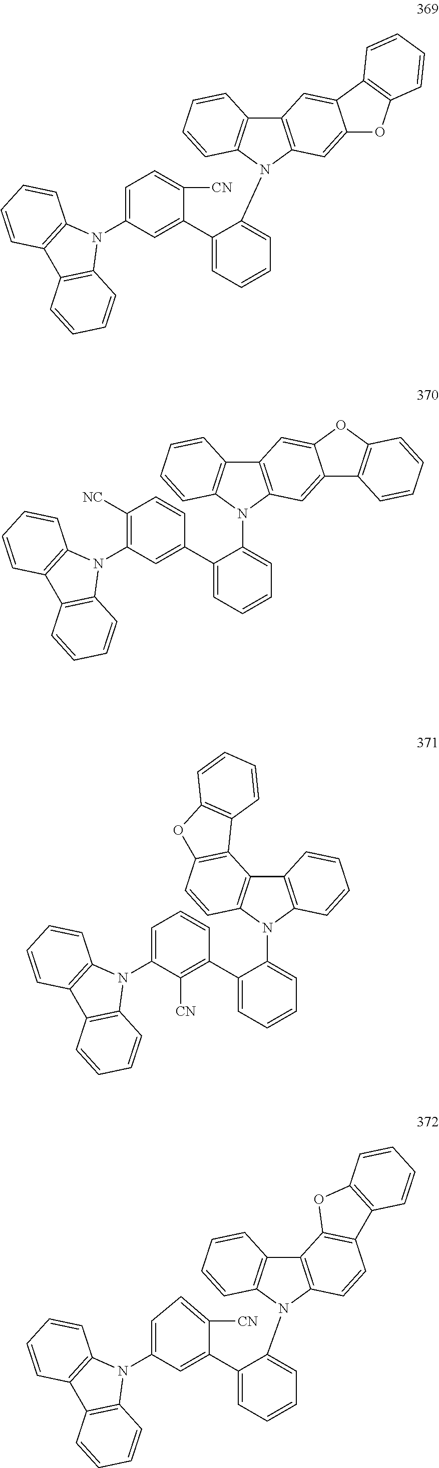 [SMILES: N#Cc1ccc(-n2c3ccccc3c3ccccc32)cc1-c1ccccc1-n1c2ccccc2c2cc3c(cc21)oc1ccccc13 ; N#Cc1ccc(-c2ccccc2-n2c3ccccc3c3cc4oc5ccccc5c4cc32)cc1-n1c2ccccc2c2ccccc21 ; N#Cc1c(-c2ccccc2-n2c3ccccc3c3c4c(ccc32)oc2ccccc24)cccc1-n1c2ccccc2c2ccccc21 ; N#Cc1ccc(-n2c3ccccc3c3ccccc32)cc1-c1ccccc1-n1c2ccccc2c2c3oc4ccccc4c3ccc21]

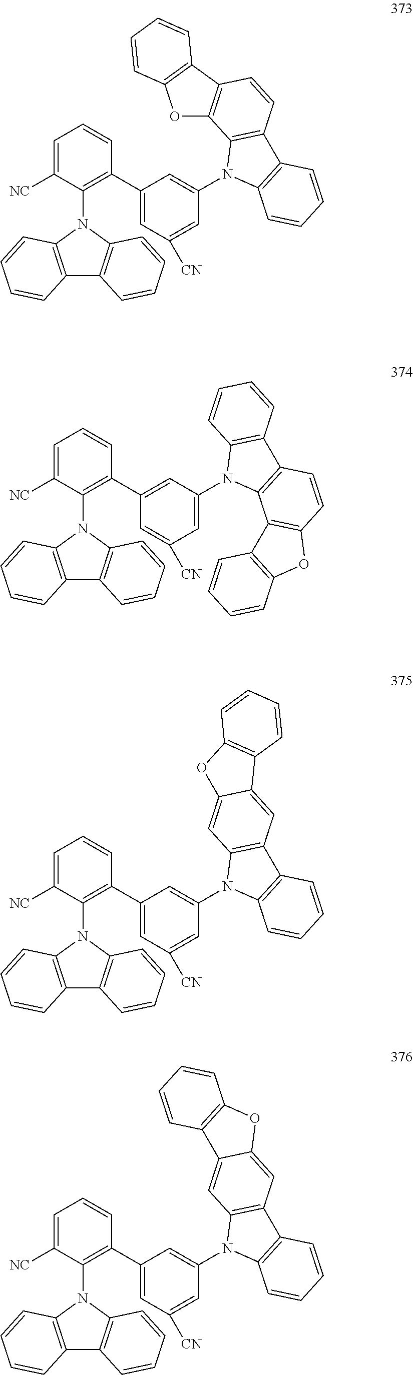 [SMILES: N#Cc1cc(-c2cccc(C#N)c2-n2c3ccccc3c3ccccc32)cc(-n2c3ccccc3c3ccc4c5ccccc5oc4c32)c1 ; N#Cc1cc(-c2cccc(C#N)c2-n2c3ccccc3c3ccccc32)cc(-n2c3ccccc3c3ccc4oc5ccccc5c4c32)c1 ; N#Cc1cc(-c2cccc(C#N)c2-n2c3ccccc3c3ccccc32)cc(-n2c3ccccc3c3cc4c(cc32)oc2ccccc24)c1 ; N#Cc1cc(-c2cccc(C#N)c2-n2c3ccccc3c3ccccc32)cc(-n2c3ccccc3c3cc4oc5ccccc5c4cc32)c1]

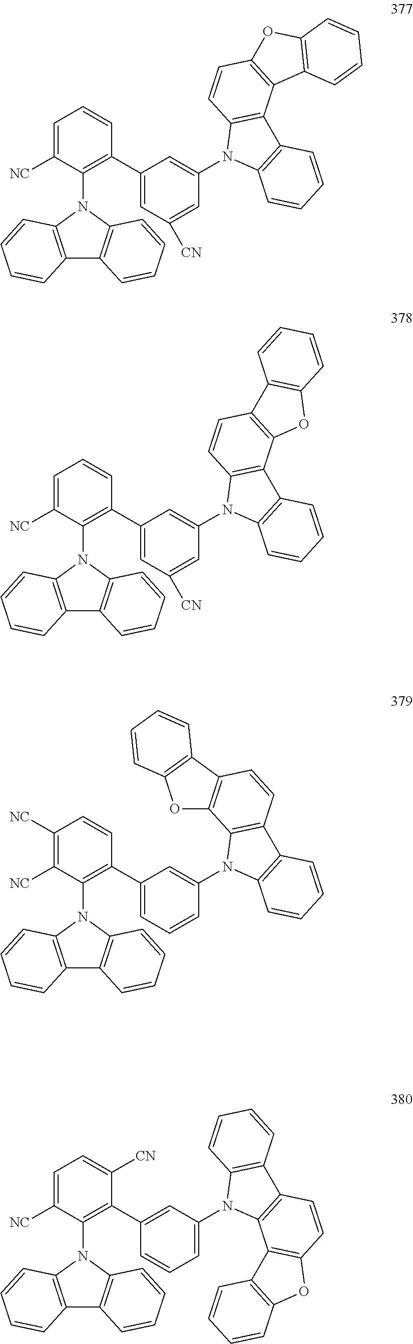 [SMILES: N#Cc1cc(-c2cccc(C#N)c2-n2c3ccccc3c3ccccc32)cc(-n2c3ccccc3c3c4c(ccc32)oc2ccccc24)c1 ; N#Cc1cc(-c2cccc(C#N)c2-n2c3ccccc3c3ccccc32)cc(-n2c3ccccc3c3c4oc5ccccc5c4ccc32)c1 ; N#Cc1ccc(-c2cccc(-n3c4ccccc4c4ccc5c6ccccc6oc5c43)c2)c(-n2c3ccccc3c3ccccc32)c1C#N ; N#Cc1ccc(C#N)c(-n2c3ccccc3c3ccccc32)c1-c1cccc(-n2c3ccccc3c3ccc4oc5ccccc5c4c32)c1]

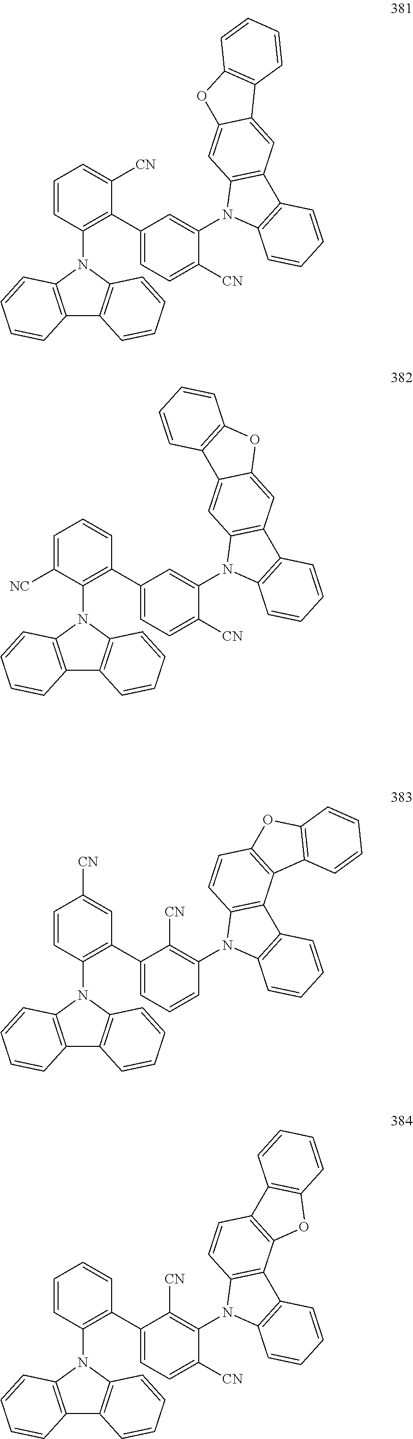 [SMILES: N#Cc1ccc(-c2c(C#N)cccc2-n2c3ccccc3c3ccccc32)cc1-n1c2ccccc2c2cc3c(cc21)oc1ccccc13 ; N#Cc1ccc(-c2cccc(C#N)c2-n2c3ccccc3c3ccccc32)cc1-n1c2ccccc2c2cc3oc4ccccc4c3cc21 ; N#Cc1ccc(-n2c3ccccc3c3ccccc32)c(-c2cccc(-n3c4ccccc4c4c5c(ccc43)oc3ccccc35)c2C#N)c1 ; N#Cc1ccc(-c2ccccc2-n2c3ccccc3c3ccccc32)c(C#N)c1-n1c2ccccc2c2c3oc4ccccc4c3ccc21]

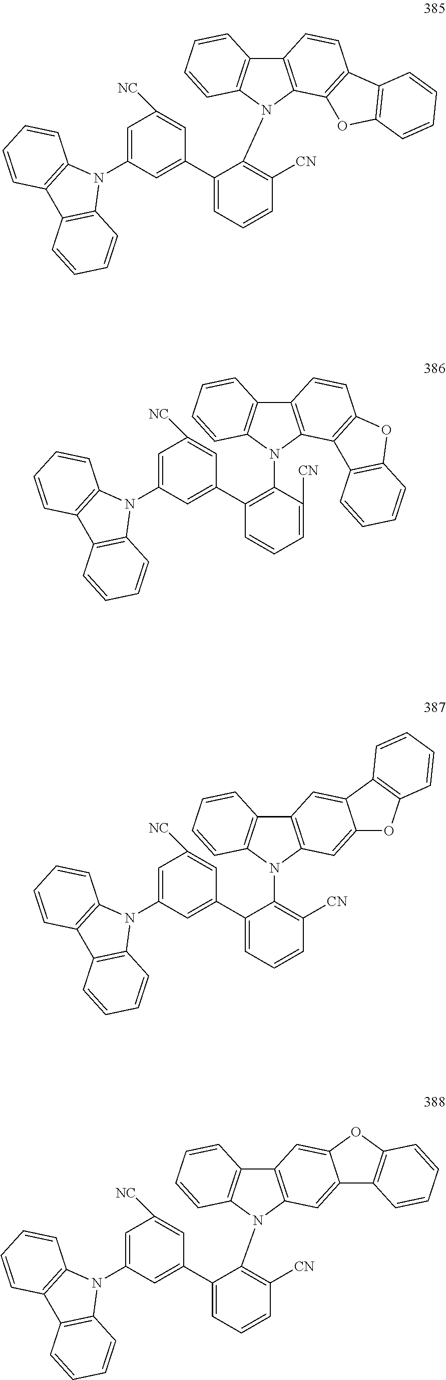 [SMILES: N#Cc1cc(-c2cccc(C#N)c2-n2c3ccccc3c3ccc4c5ccccc5oc4c32)cc(-n2c3ccccc3c3ccccc32)c1 ; N#Cc1cc(-c2cccc(C#N)c2-n2c3ccccc3c3ccc4oc5ccccc5c4c32)cc(-n2c3ccccc3c3ccccc32)c1 ; N#Cc1cc(-c2cccc(C#N)c2-n2c3ccccc3c3cc4c(cc32)oc2ccccc24)cc(-n2c3ccccc3c3ccccc32)c1 ; N#Cc1cc(-c2cccc(C#N)c2-n2c3ccccc3c3cc4oc5ccccc5c4cc32)cc(-n2c3ccccc3c3ccccc32)c1]

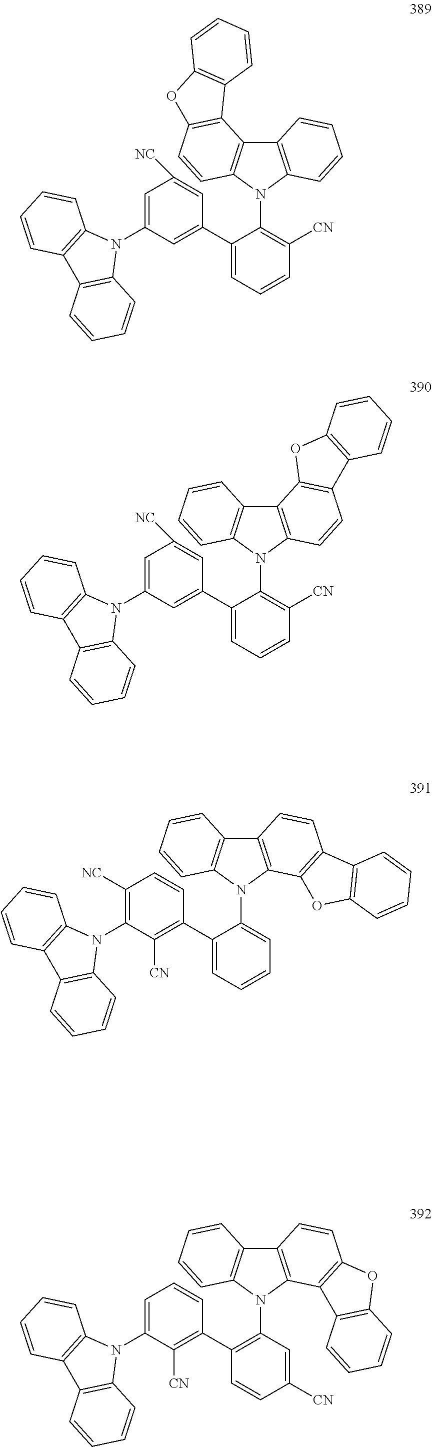 [SMILES: N#Cc1cc(-c2cccc(C#N)c2-n2c3ccccc3c3c4c(ccc32)oc2ccccc24)cc(-n2c3ccccc3c3ccccc32)c1 ; N#Cc1cc(-c2cccc(C#N)c2-n2c3ccccc3c3c4oc5ccccc5c4ccc32)cc(-n2c3ccccc3c3ccccc32)c1 ; N#Cc1ccc(-c2ccccc2-n2c3ccccc3c3ccc4c5ccccc5oc4c32)c(C#N)c1-n1c2ccccc2c2ccccc21 ; N#Cc1ccc(-c2cccc(-n3c4ccccc4c4ccccc43)c2C#N)c(-n2c3ccccc3c3ccc4oc5ccccc5c4c32)c1]

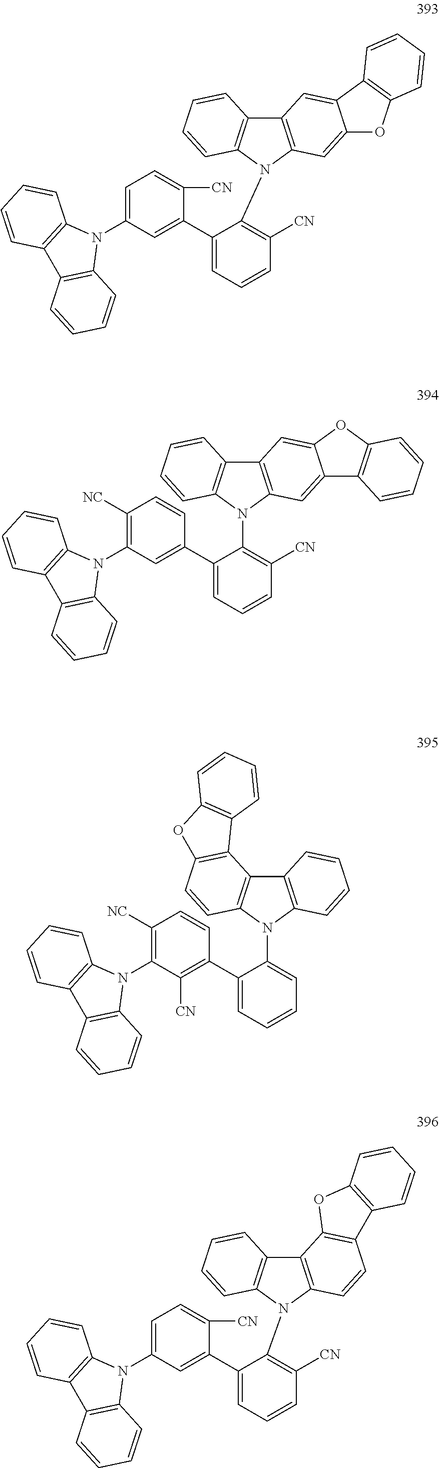 [SMILES: N#Cc1ccc(-n2c3ccccc3c3ccccc32)cc1-c1cccc(C#N)c1-n1c2ccccc2c2cc3c(cc21)oc1ccccc13 ; N#Cc1ccc(-c2cccc(C#N)c2-n2c3ccccc3c3cc4oc5ccccc5c4cc32)cc1-n1c2ccccc2c2ccccc21 ; N#Cc1ccc(-c2ccccc2-n2c3ccccc3c3c4c(ccc32)oc2ccccc24)c(C#N)c1-n1c2ccccc2c2ccccc21 ; N#Cc1ccc(-n2c3ccccc3c3ccccc32)cc1-c1cccc(C#N)c1-n1c2ccccc2c2c3oc4ccccc4c3ccc21]

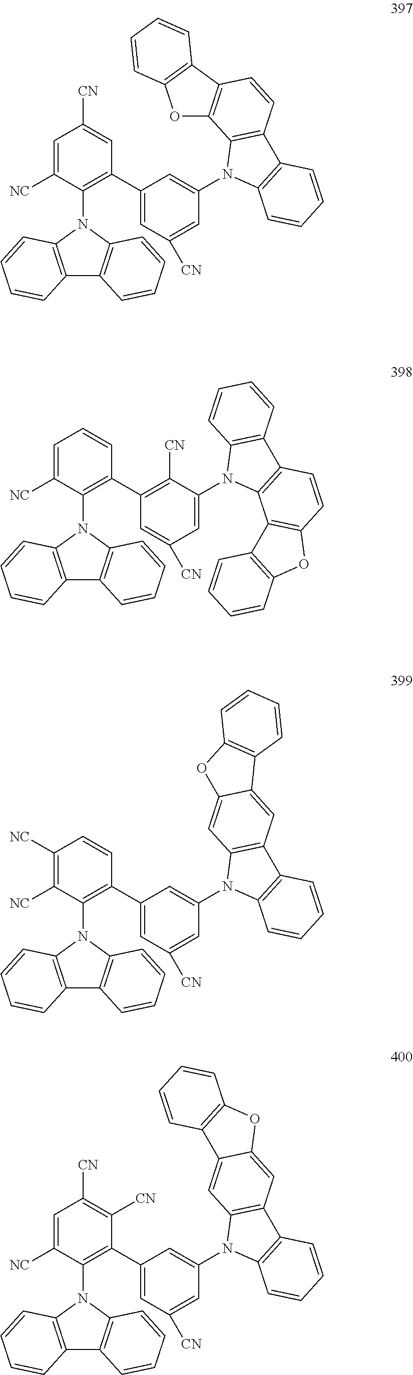 [SMILES: N#Cc1cc(-c2cc(C#N)cc(C#N)c2-n2c3ccccc3c3ccccc32)cc(-n2c3ccccc3c3ccc4c5ccccc5oc4c32)c1 ; N#Cc1cc(-c2cccc(C#N)c2-n2c3ccccc3c3ccccc32)c(C#N)c(-n2c3ccccc3c3ccc4oc5ccccc5c4c32)c1 ; N#Cc1cc(-c2ccc(C#N)c(C#N)c2-n2c3ccccc3c3ccccc32)cc(-n2c3ccccc3c3cc4c(cc32)oc2ccccc24)c1 ; N#Cc1cc(-c2c(C#N)c(C#N)cc(C#N)c2-n2c3ccccc3c3ccccc32)cc(-n2c3ccccc3c3cc4oc5ccccc5c4cc32)c1]

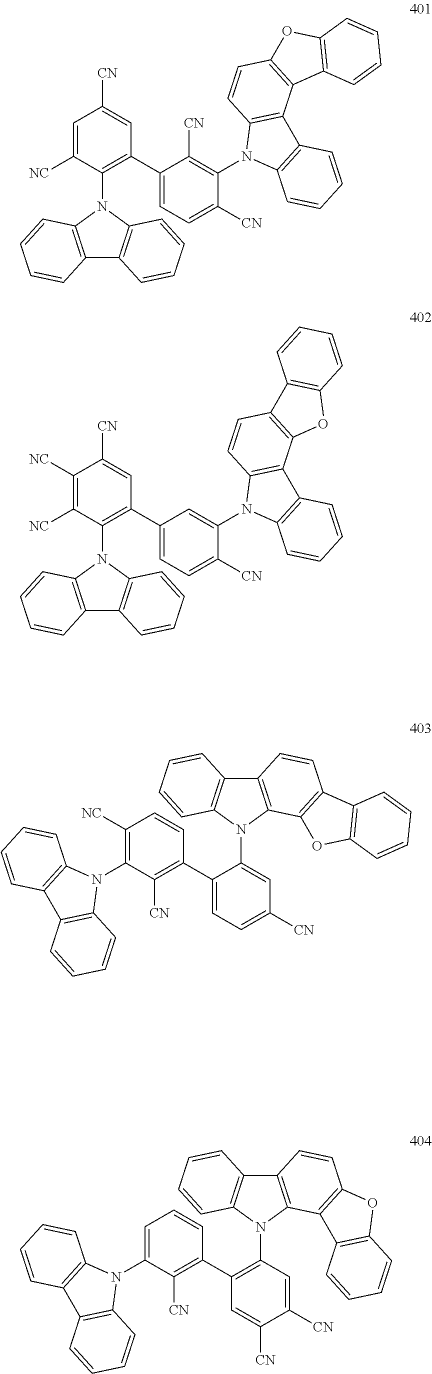 [SMILES: N#Cc1cc(C#N)c(-n2c3ccccc3c3ccccc32)c(-c2ccc(C#N)c(-n3c4ccccc4c4c5c(ccc43)oc3ccccc35)c2C#N)c1 ; N#Cc1ccc(-c2cc(C#N)c(C#N)c(C#N)c2-n2c3ccccc3c3ccccc32)cc1-n1c2ccccc2c2c3oc4ccccc4c3ccc21 ; N#Cc1ccc(-c2ccc(C#N)c(-n3c4ccccc4c4ccccc43)c2C#N)c(-n2c3ccccc3c3ccc4c5ccccc5oc4c32)c1 ; N#Cc1cc(-c2cccc(-n3c4ccccc4c4ccccc43)c2C#N)c(-n2c3ccccc3c3ccc4oc5ccccc5c4c32)cc1C#N]

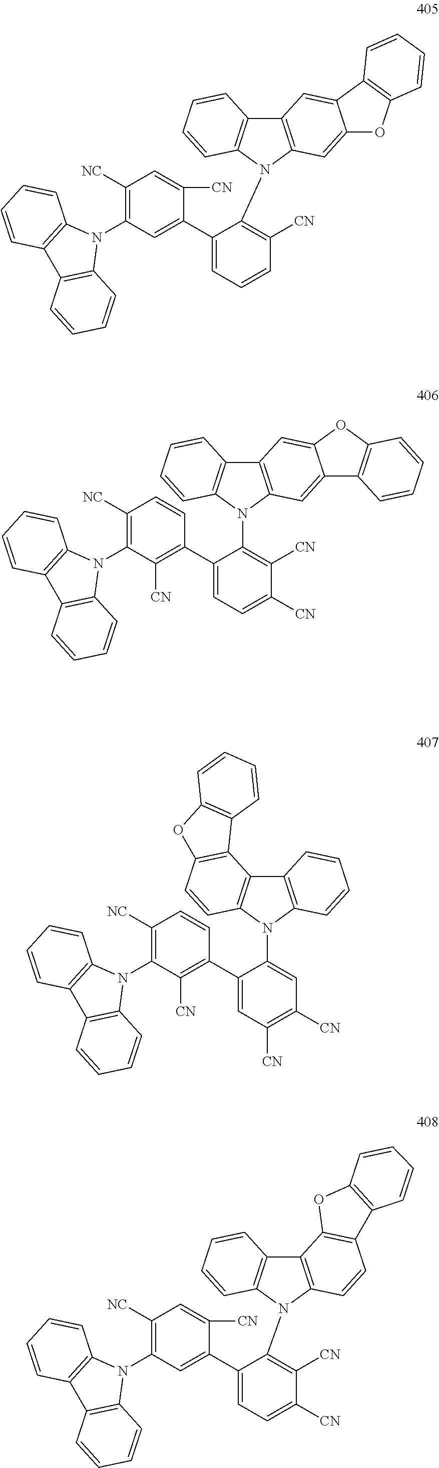 [SMILES: N#Cc1cc(C#N)c(-n2c3ccccc3c3ccccc32)cc1-c1cccc(C#N)c1-n1c2ccccc2c2cc3c(cc21)oc1ccccc13 ; N#Cc1ccc(-c2ccc(C#N)c(-n3c4ccccc4c4ccccc43)c2C#N)c(-n2c3ccccc3c3cc4oc5ccccc5c4cc32)c1C#N ; N#Cc1cc(-c2ccc(C#N)c(-n3c4ccccc4c4ccccc43)c2C#N)c(-n2c3ccccc3c3c4c(ccc32)oc2ccccc24)cc1C#N ; N#Cc1cc(C#N)c(-n2c3ccccc3c3ccccc32)cc1-c1ccc(C#N)c(C#N)c1-n1c2ccccc2c2c3oc4ccccc4c3ccc21]

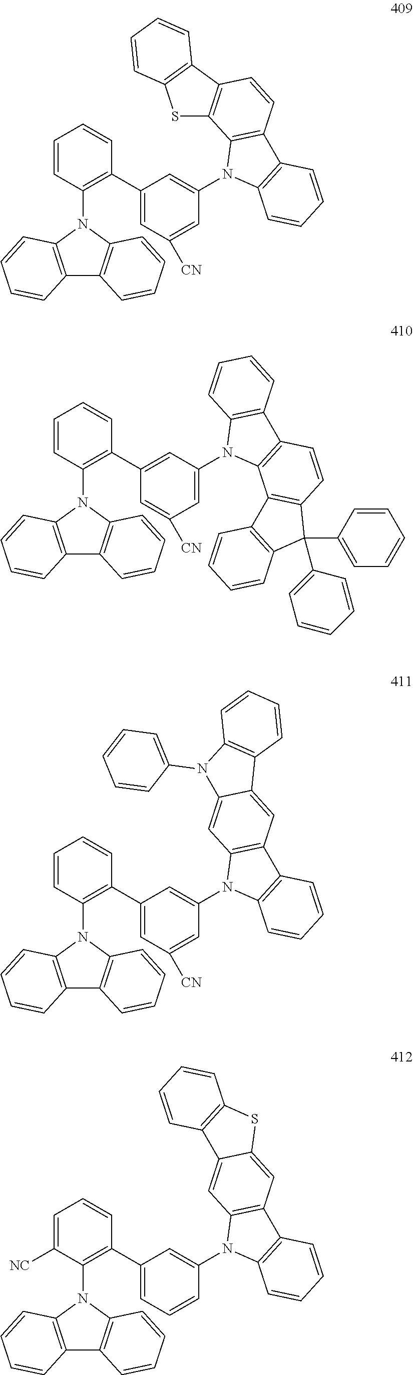 [SMILES: N#Cc1cc(-c2ccccc2-n2c3ccccc3c3ccccc32)cc(-n2c3ccccc3c3ccc4c5ccccc5sc4c32)c1 ; N#Cc1cc(-c2ccccc2-n2c3ccccc3c3ccccc32)cc(-n2c3ccccc3c3ccc4c(c32)-c2ccccc2C4(c2ccccc2)c2ccccc2)c1 ; N#Cc1cc(-c2ccccc2-n2c3ccccc3c3ccccc32)cc(-n2c3ccccc3c3cc4c5ccccc5n(-c5ccccc5)c4cc32)c1 ; N#Cc1cccc(-c2cccc(-n3c4ccccc4c4cc5sc6ccccc6c5cc43)c2)c1-n1c2ccccc2c2ccccc21]

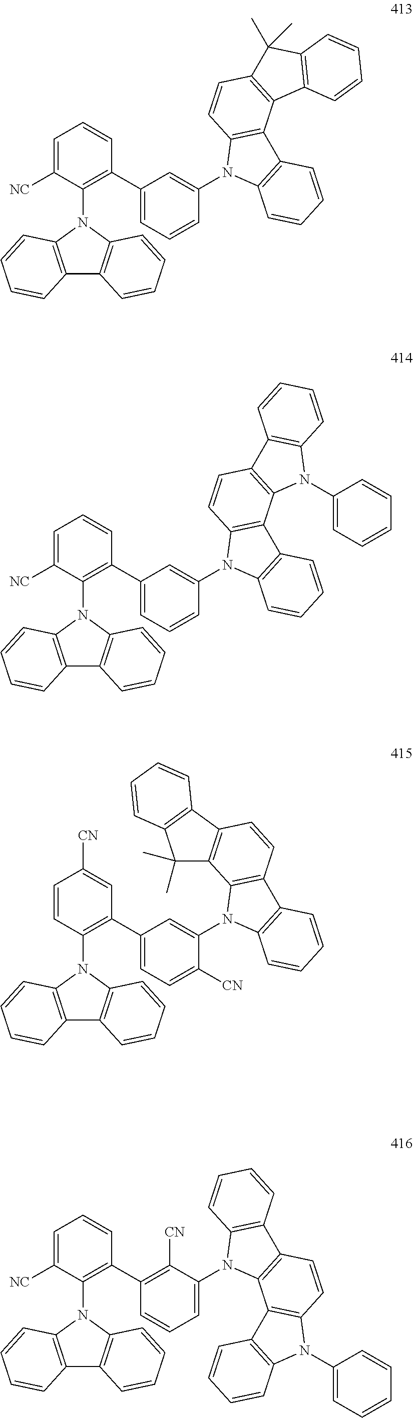 [SMILES: CC1(C)c2ccccc2-c2c1ccc1c2c2ccccc2n1-c1cccc(-c2cccc(C#N)c2-n2c3ccccc3c3ccccc32)c1 ; N#Cc1cccc(-c2cccc(-n3c4ccccc4c4c3ccc3c5ccccc5n(-c5ccccc5)c34)c2)c1-n1c2ccccc2c2ccccc21 ; CC1(C)c2ccccc2-c2ccc3c4ccccc4n(-c4cc(-c5cc(C#N)ccc5-n5c6ccccc6c6ccccc65)ccc4C#N)c3c21 ; N#Cc1cccc(-c2cccc(-n3c4ccccc4c4ccc5c(c6ccccc6n5-c5ccccc5)c43)c2C#N)c1-n1c2ccccc2c2ccccc21]

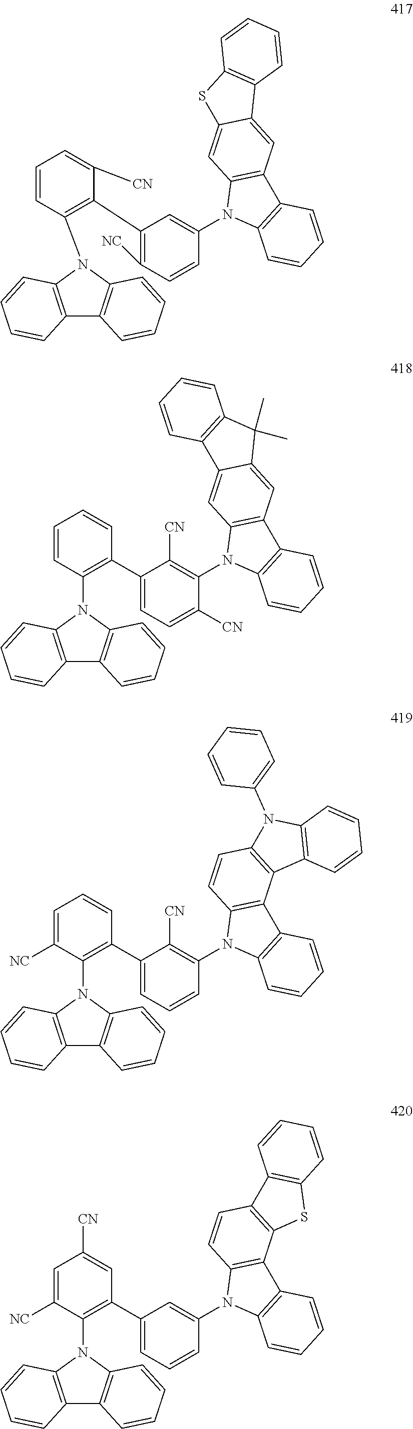 [SMILES: N#Cc1ccc(-n2c3ccccc3c3cc4c(cc32)sc2ccccc24)cc1-c1c(C#N)cccc1-n1c2ccccc2c2ccccc21 ; CC1(C)c2ccccc2-c2cc3c(cc21)c1ccccc1n3-c1c(C#N)ccc(-c2ccccc2-n2c3ccccc3c3ccccc32)c1C#N ; N#Cc1cccc(-c2cccc(-n3c4ccccc4c4c5c6ccccc6n(-c6ccccc6)c5ccc43)c2C#N)c1-n1c2ccccc2c2ccccc21 ; N#Cc1cc(C#N)c(-n2c3ccccc3c3ccccc32)c(-c2cccc(-n3c4ccccc4c4c5sc6ccccc6c5ccc43)c2)c1]

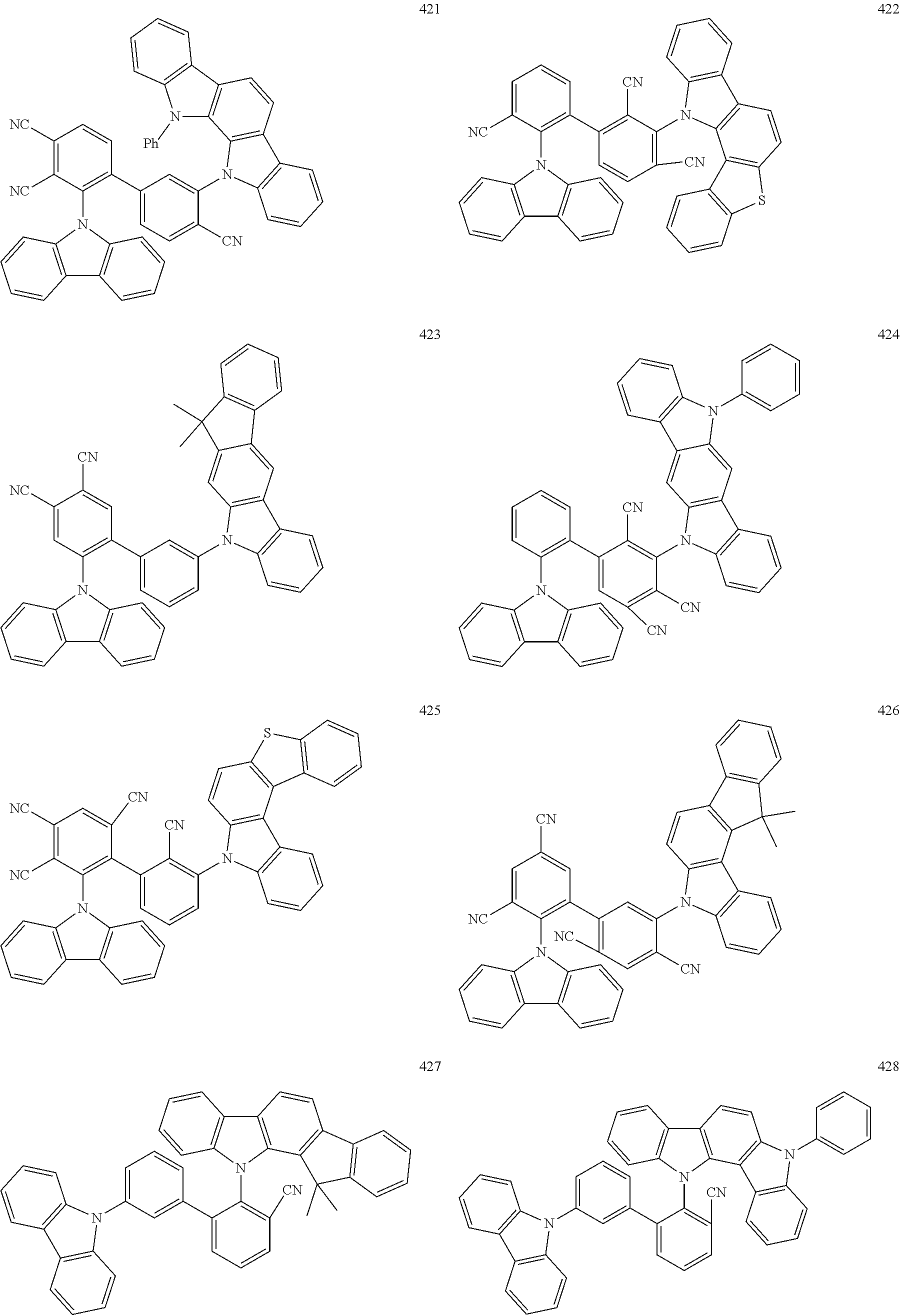

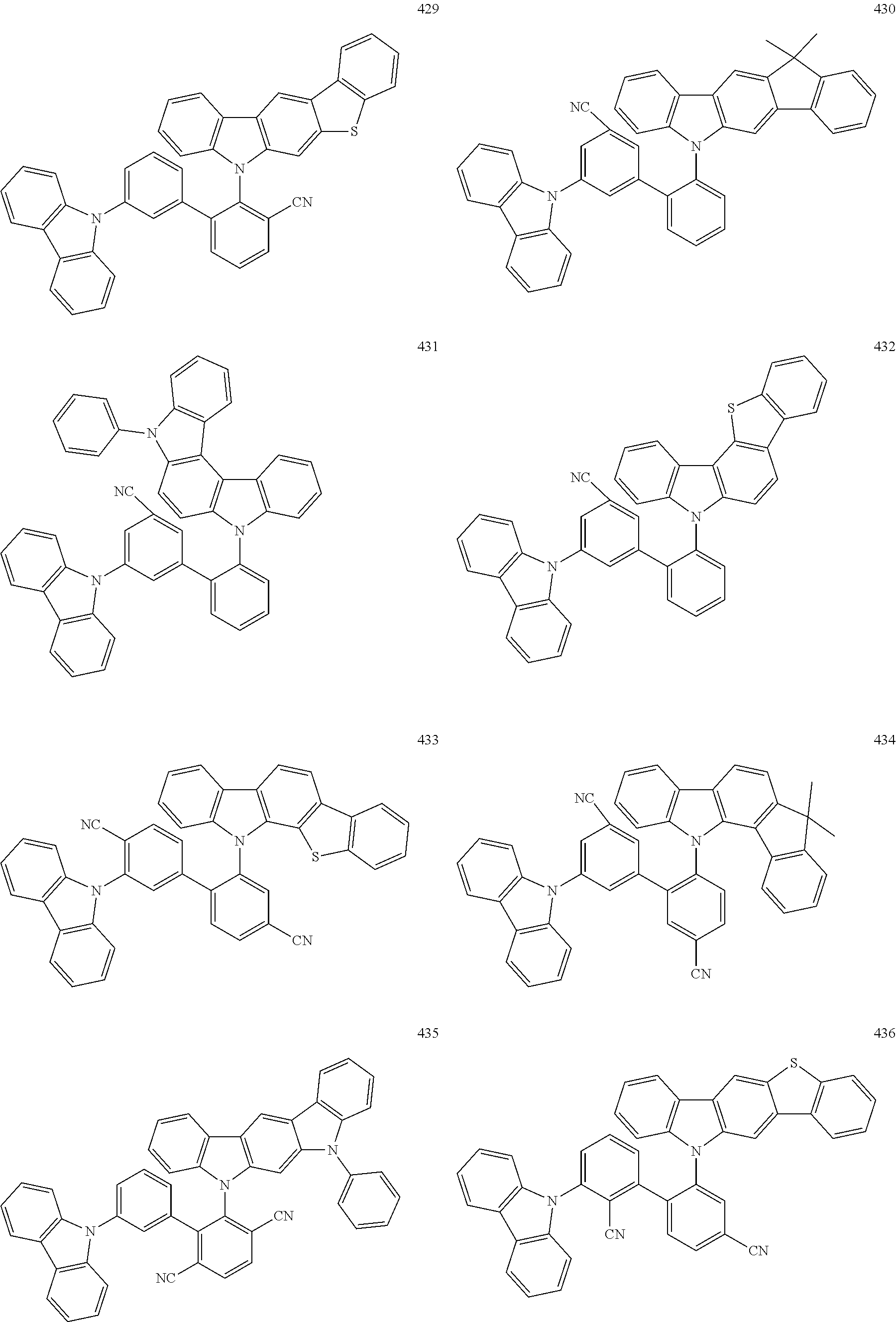

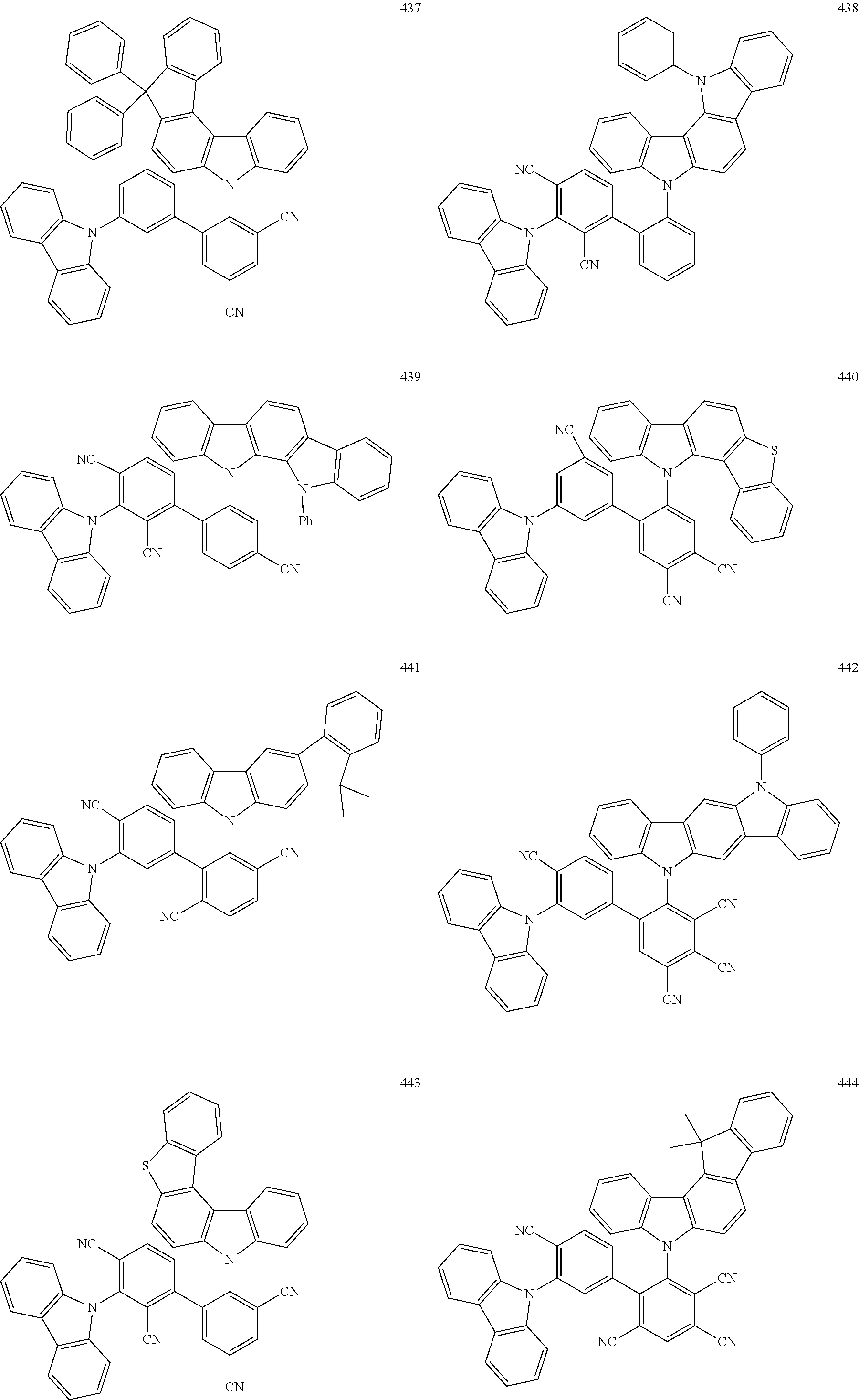

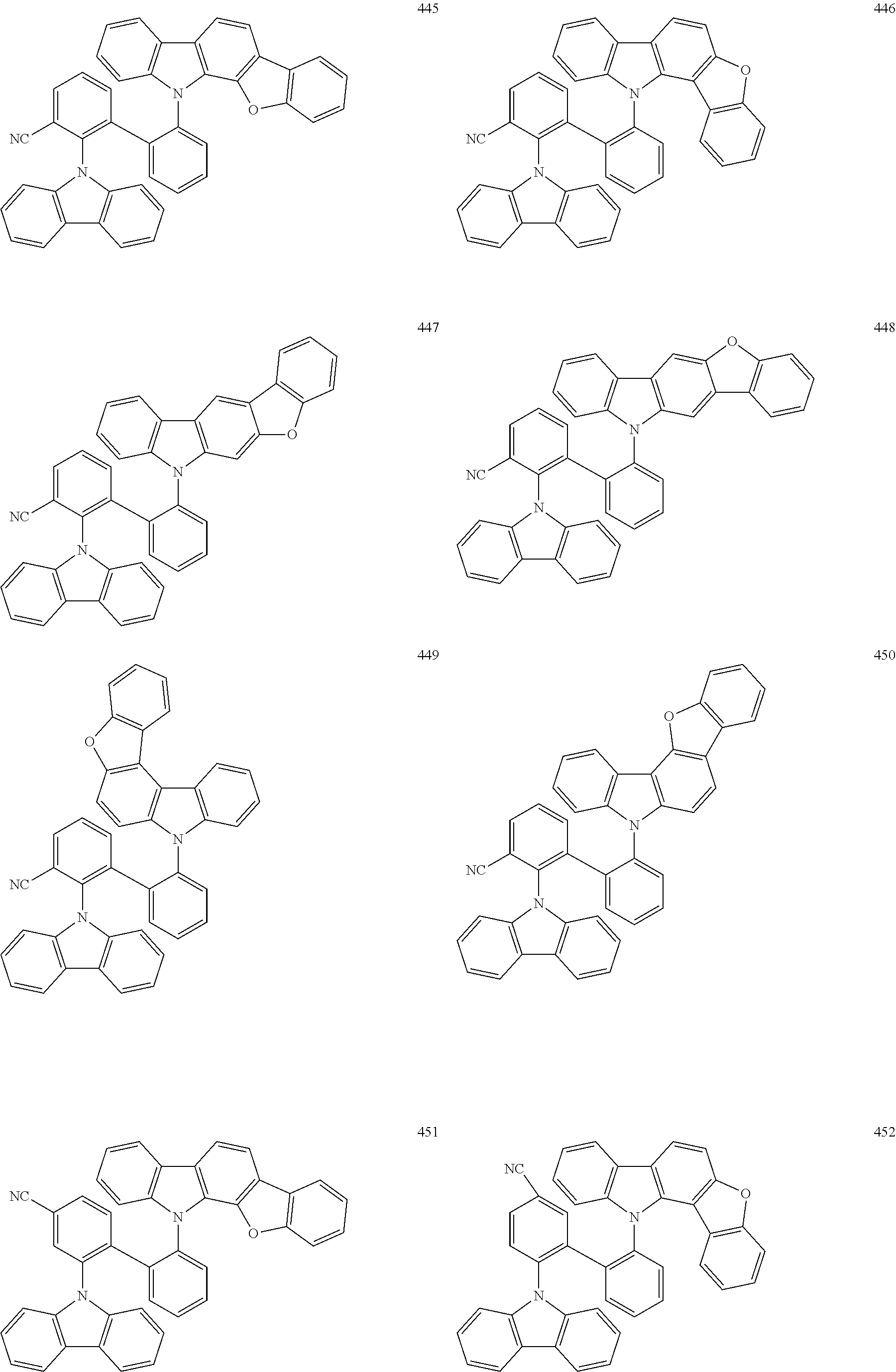

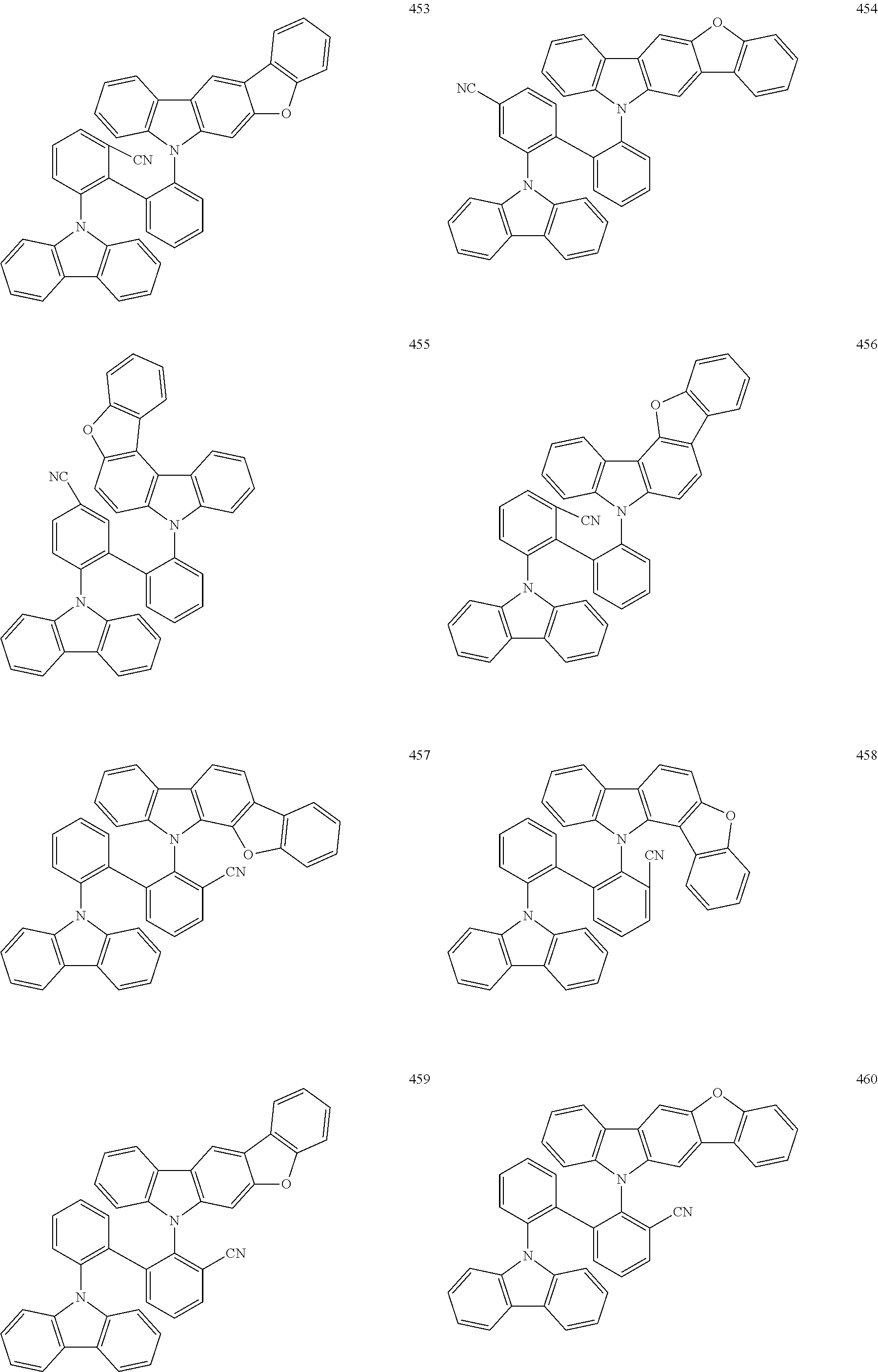

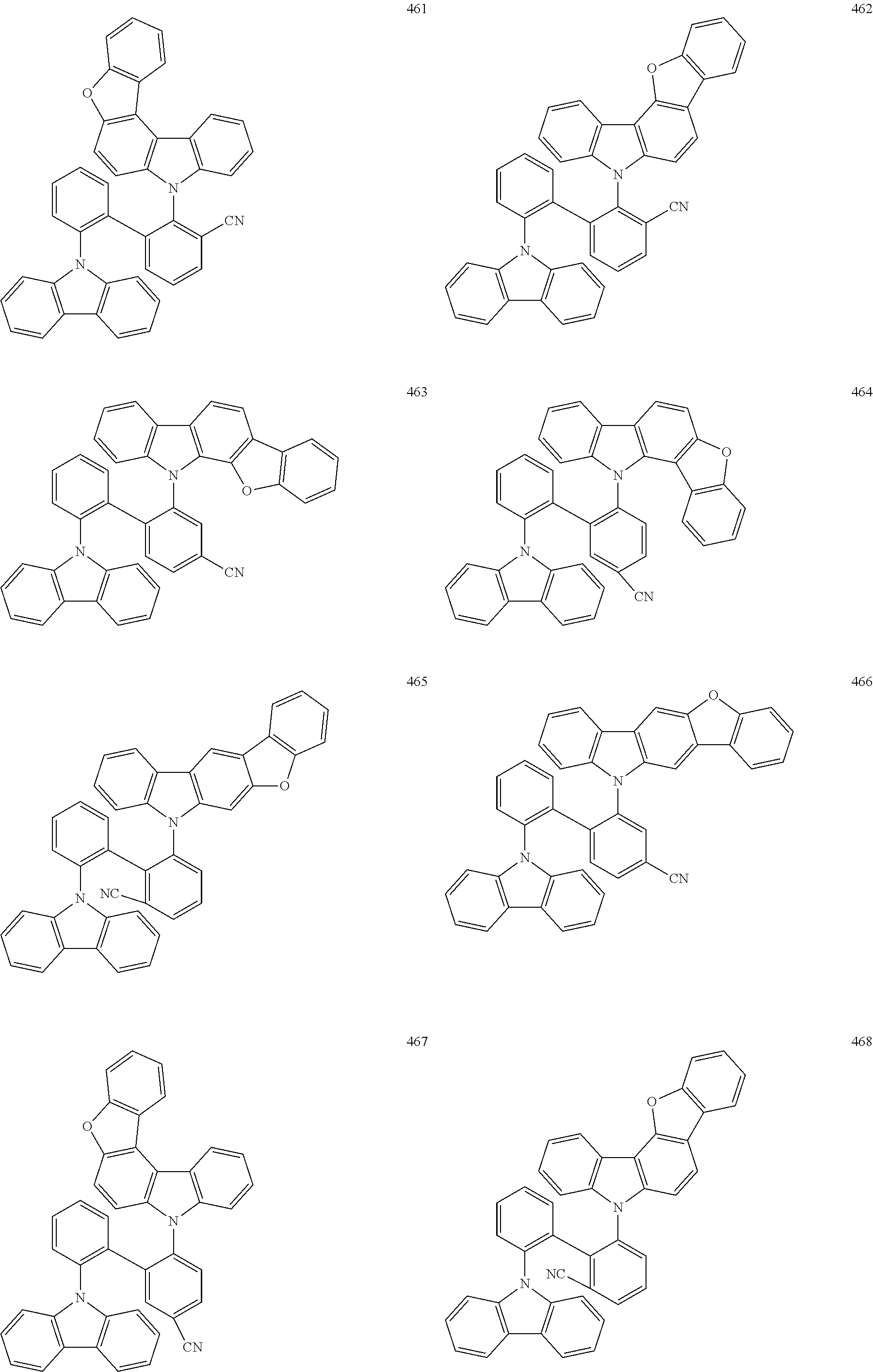

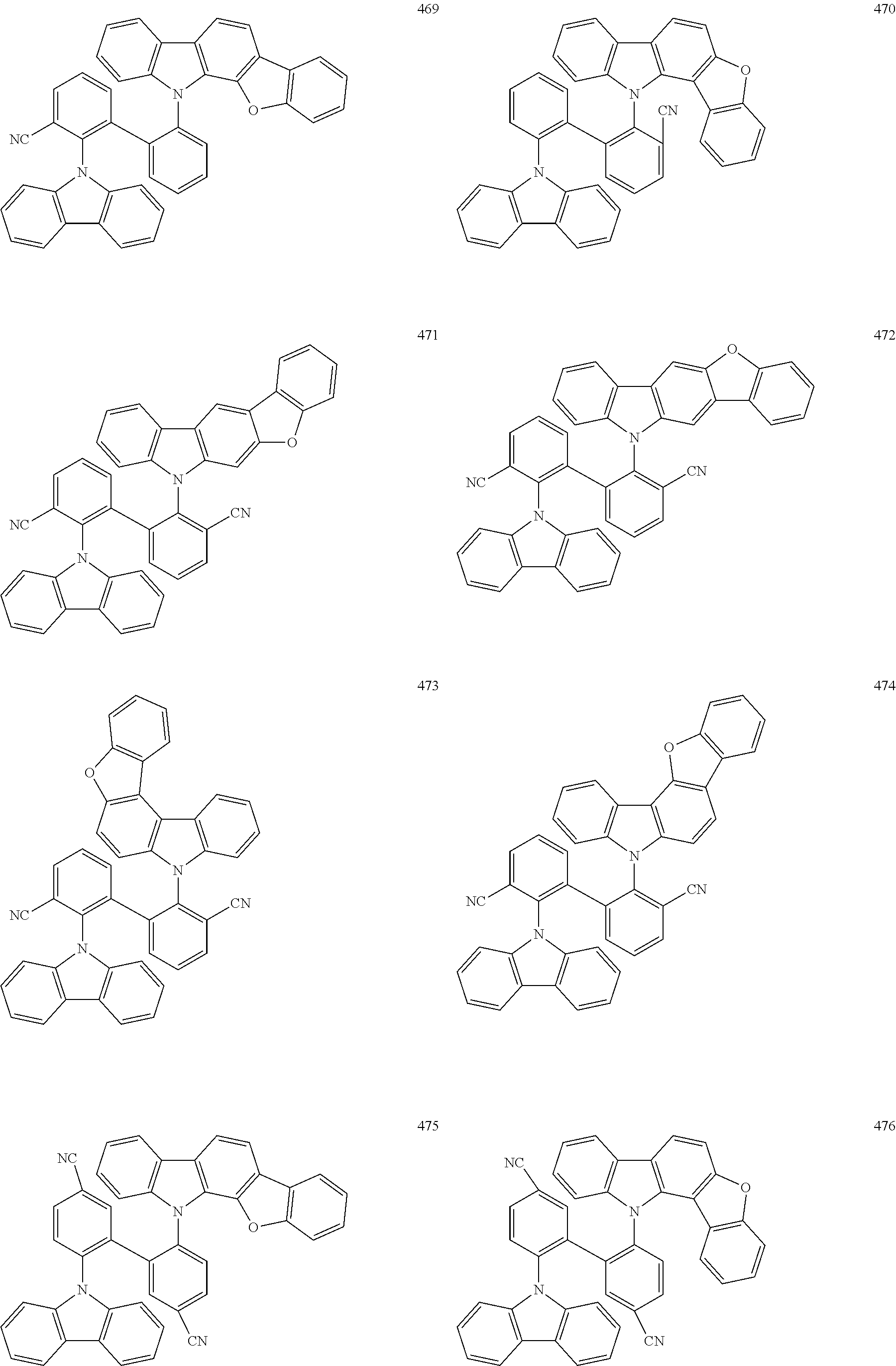

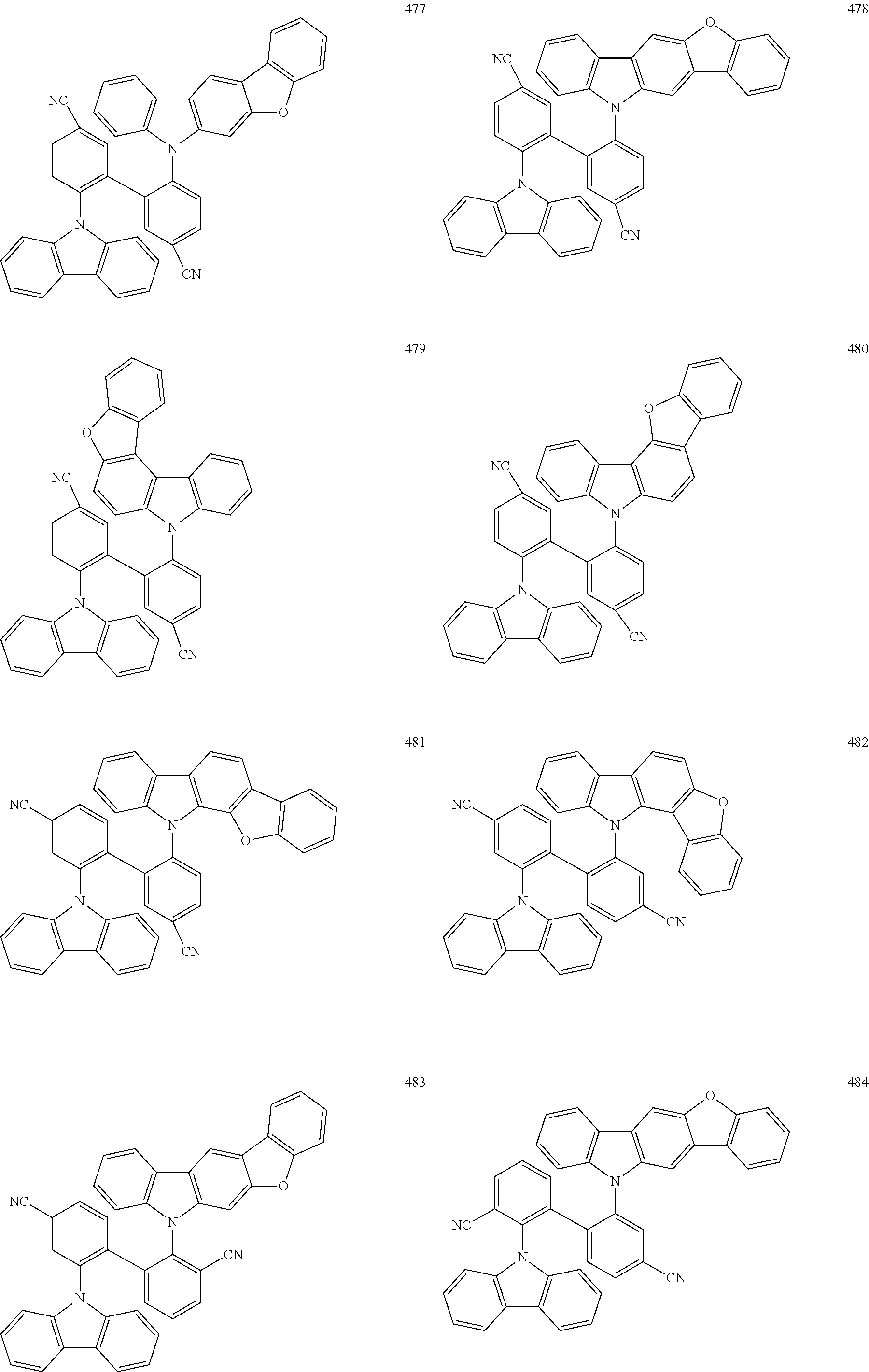

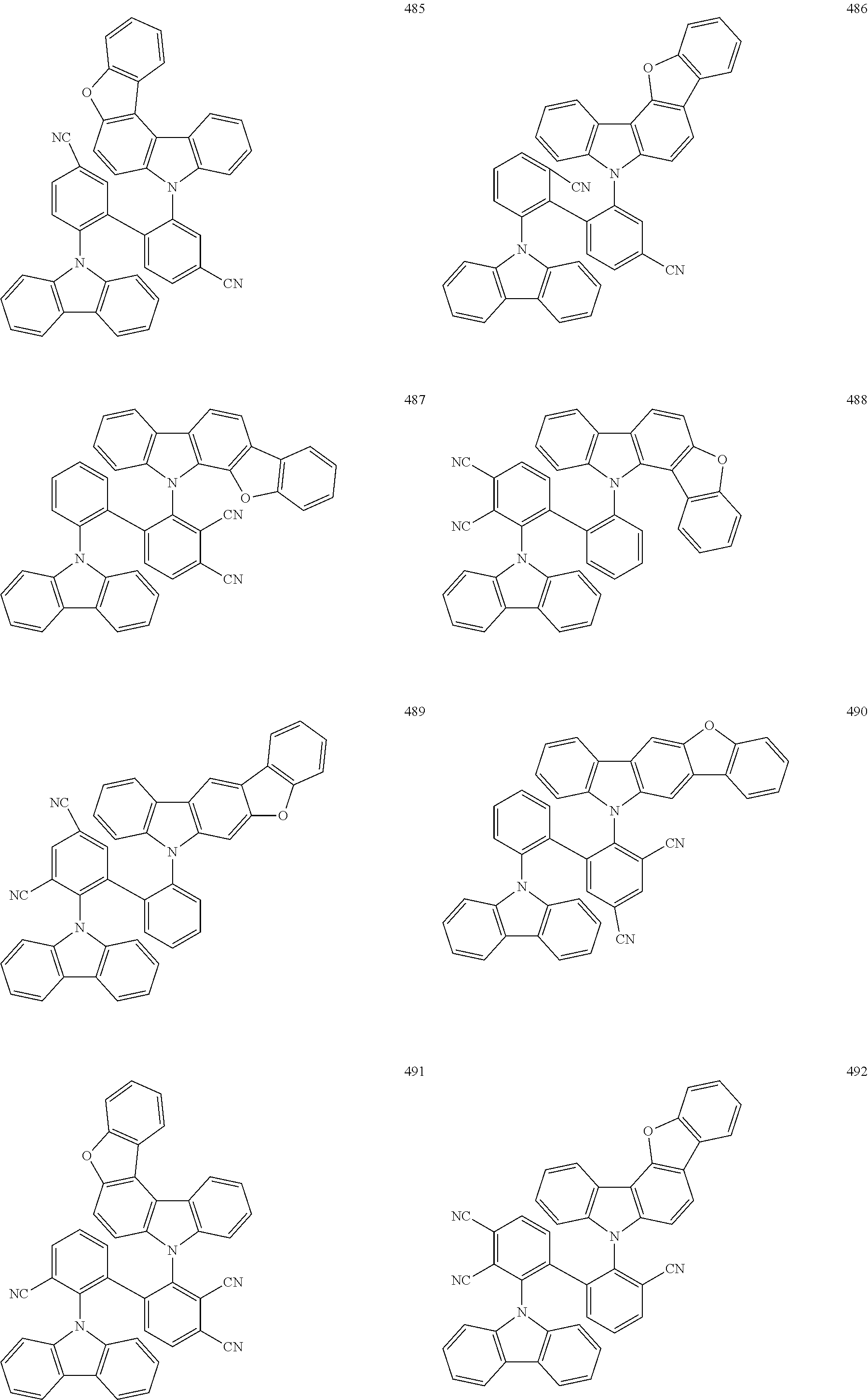

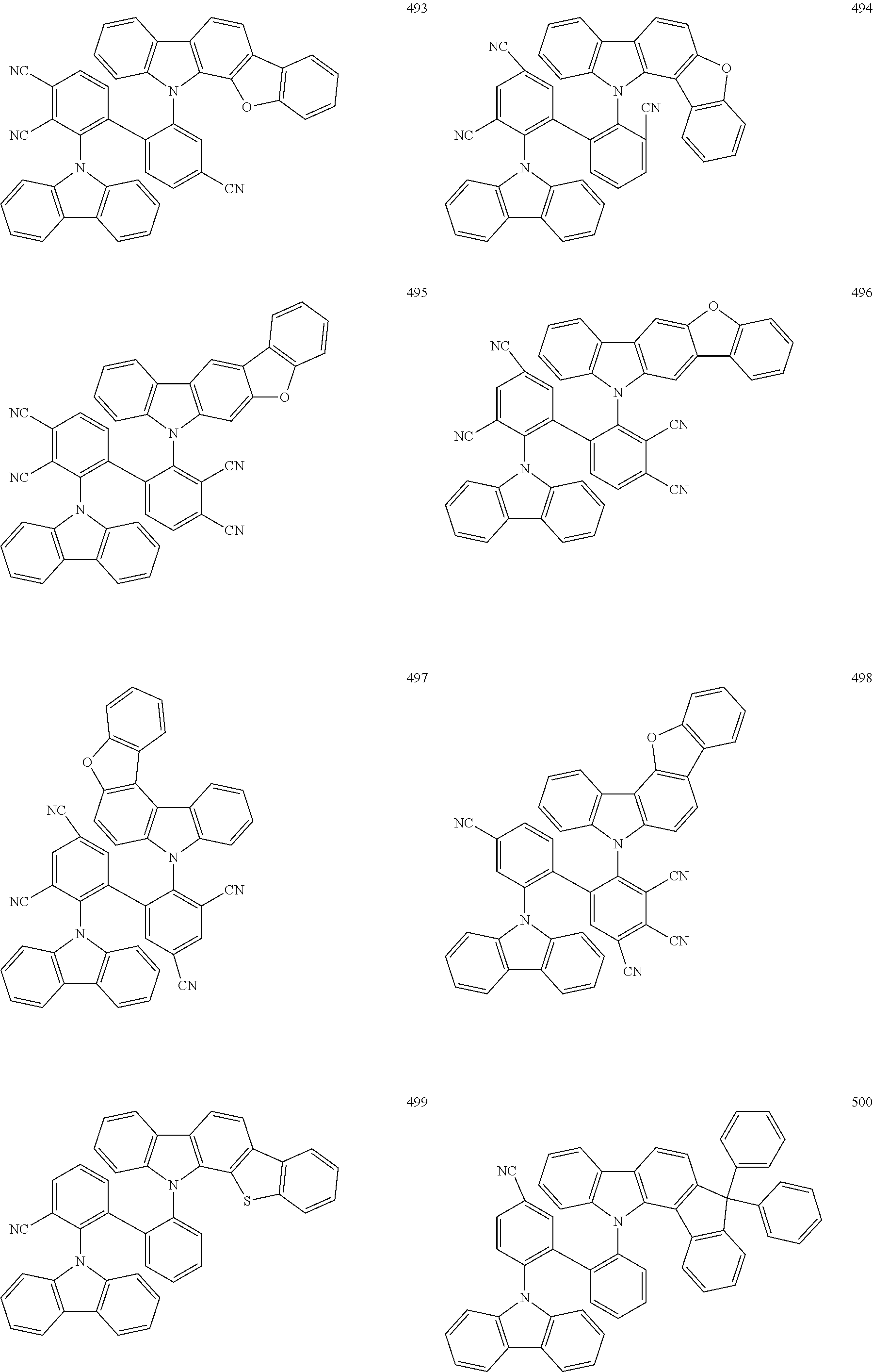

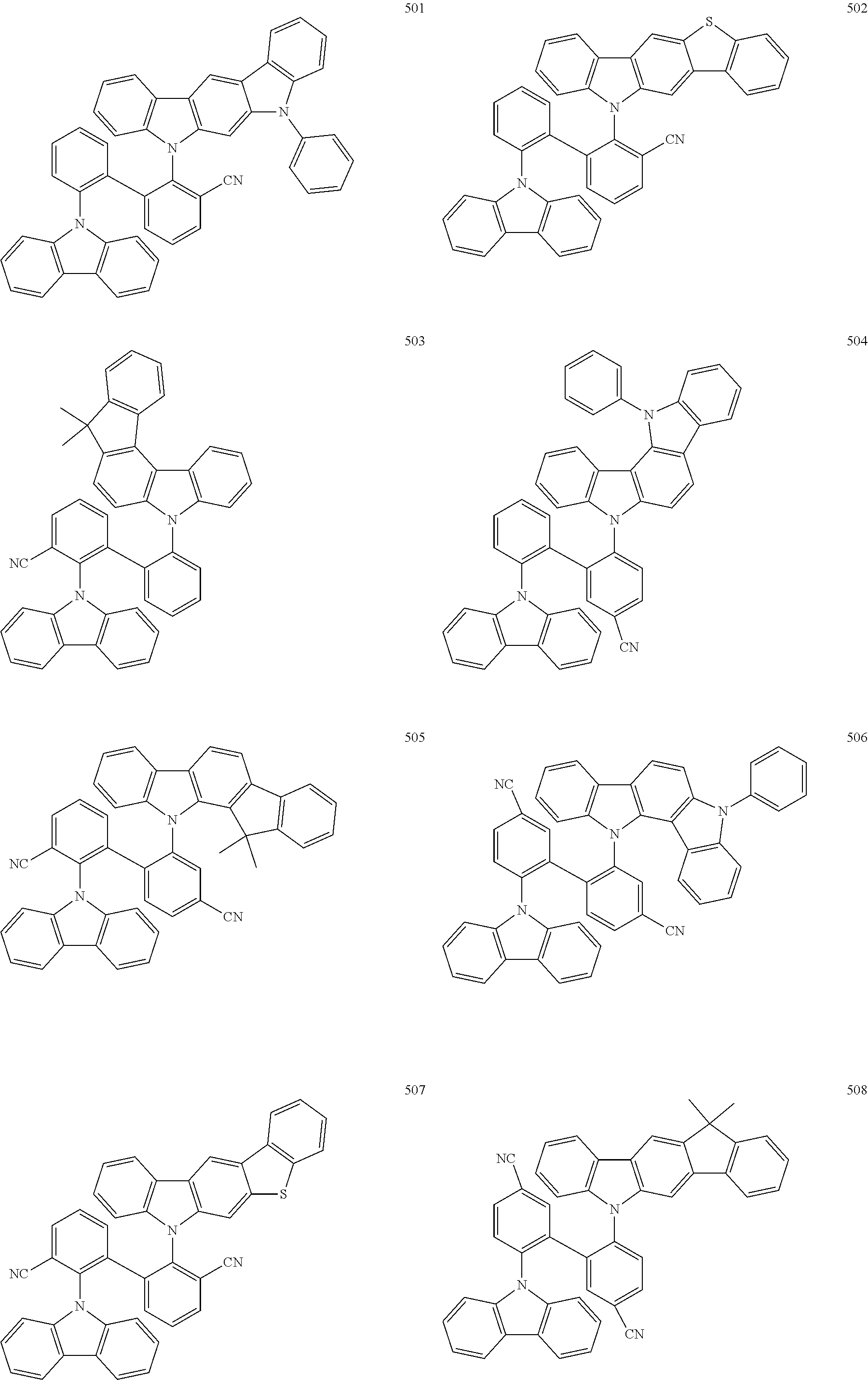

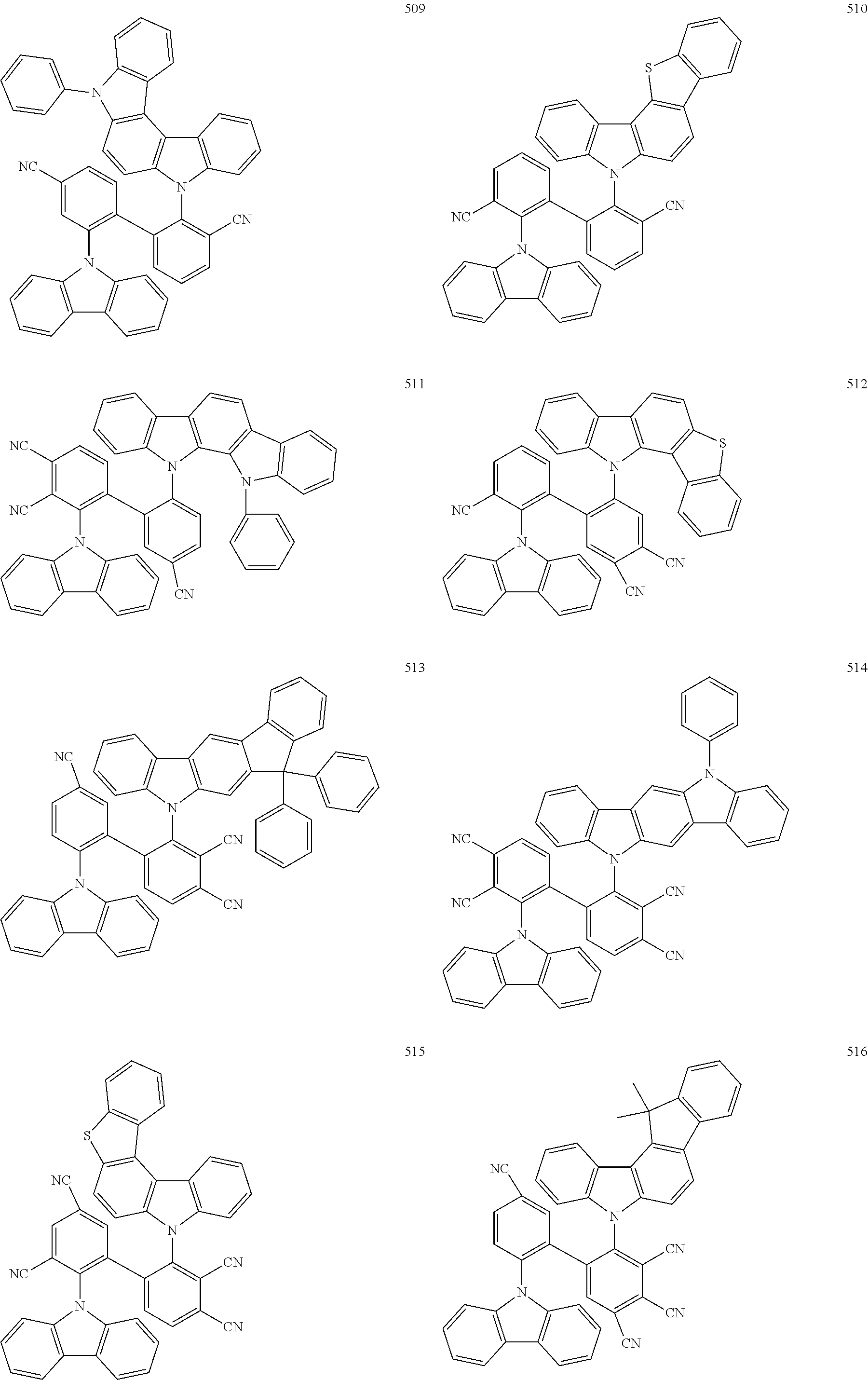

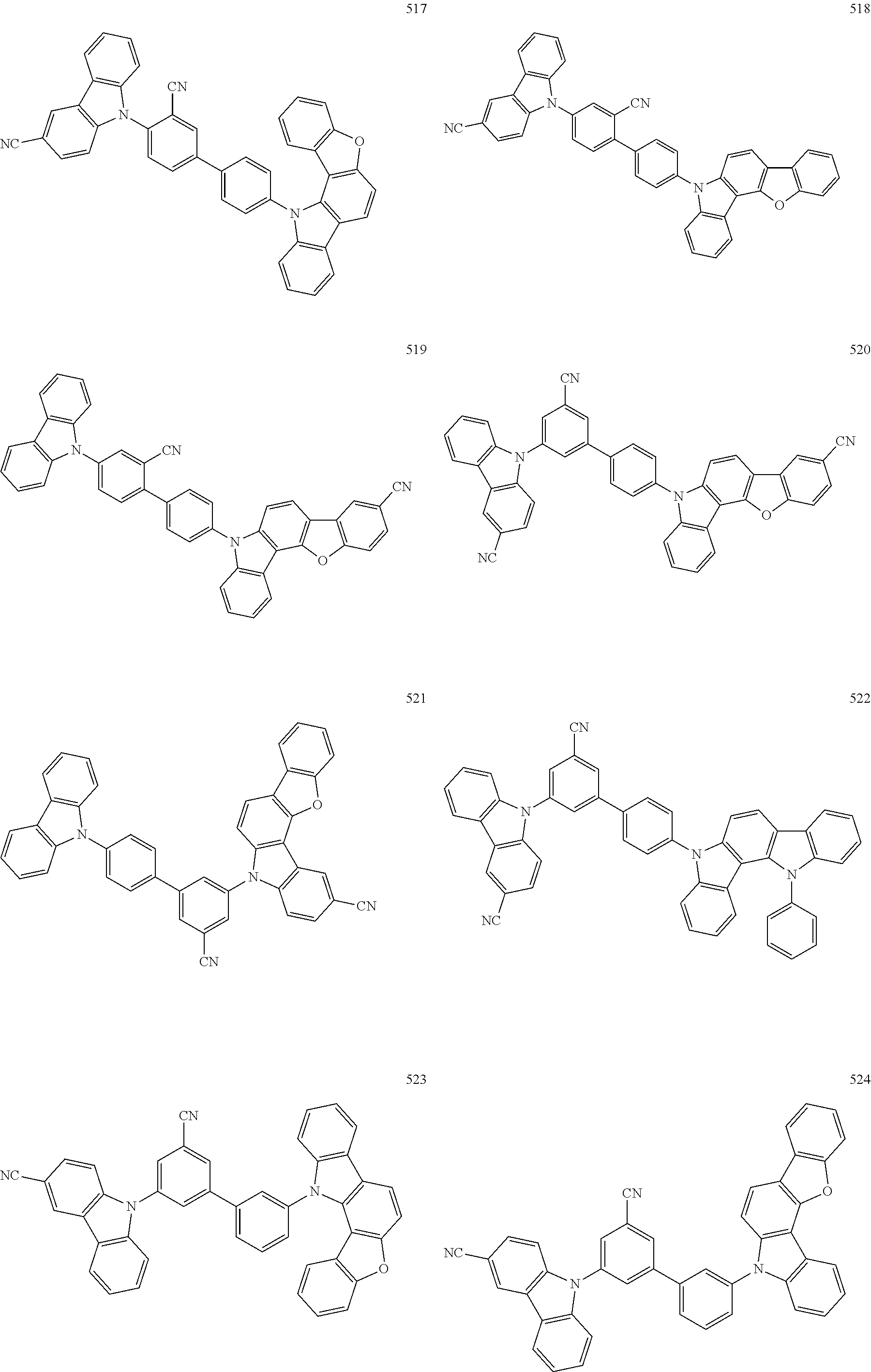

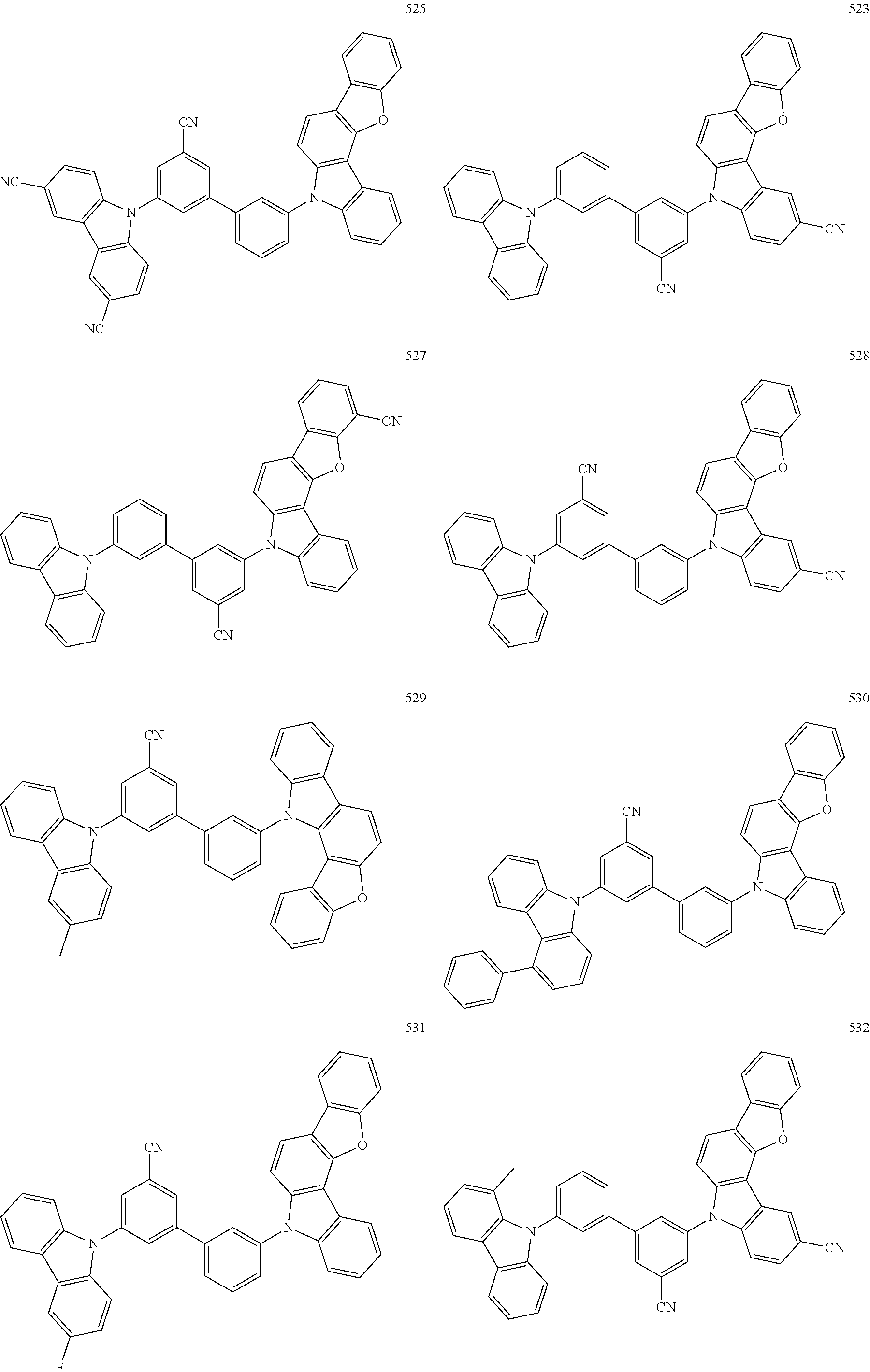

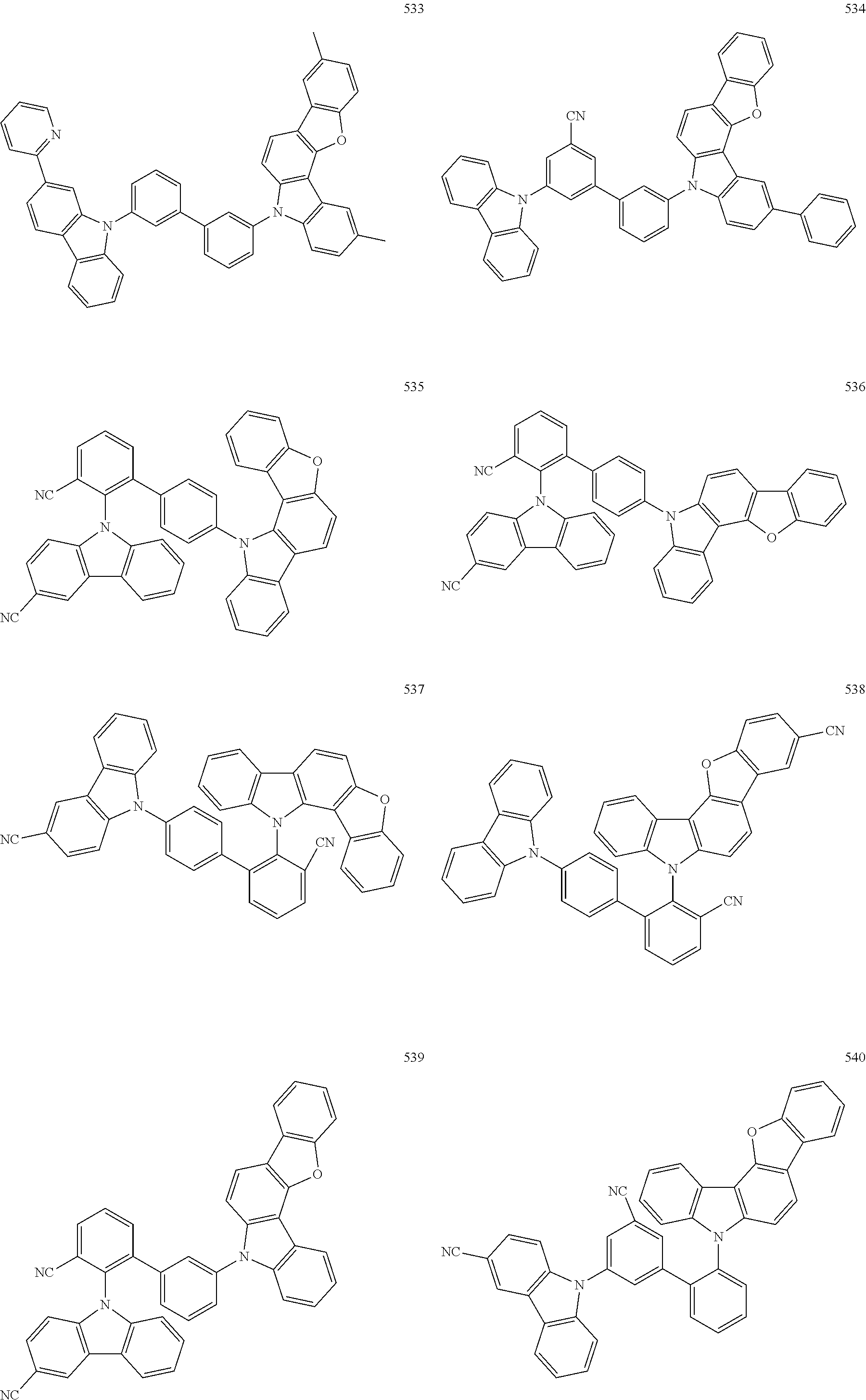

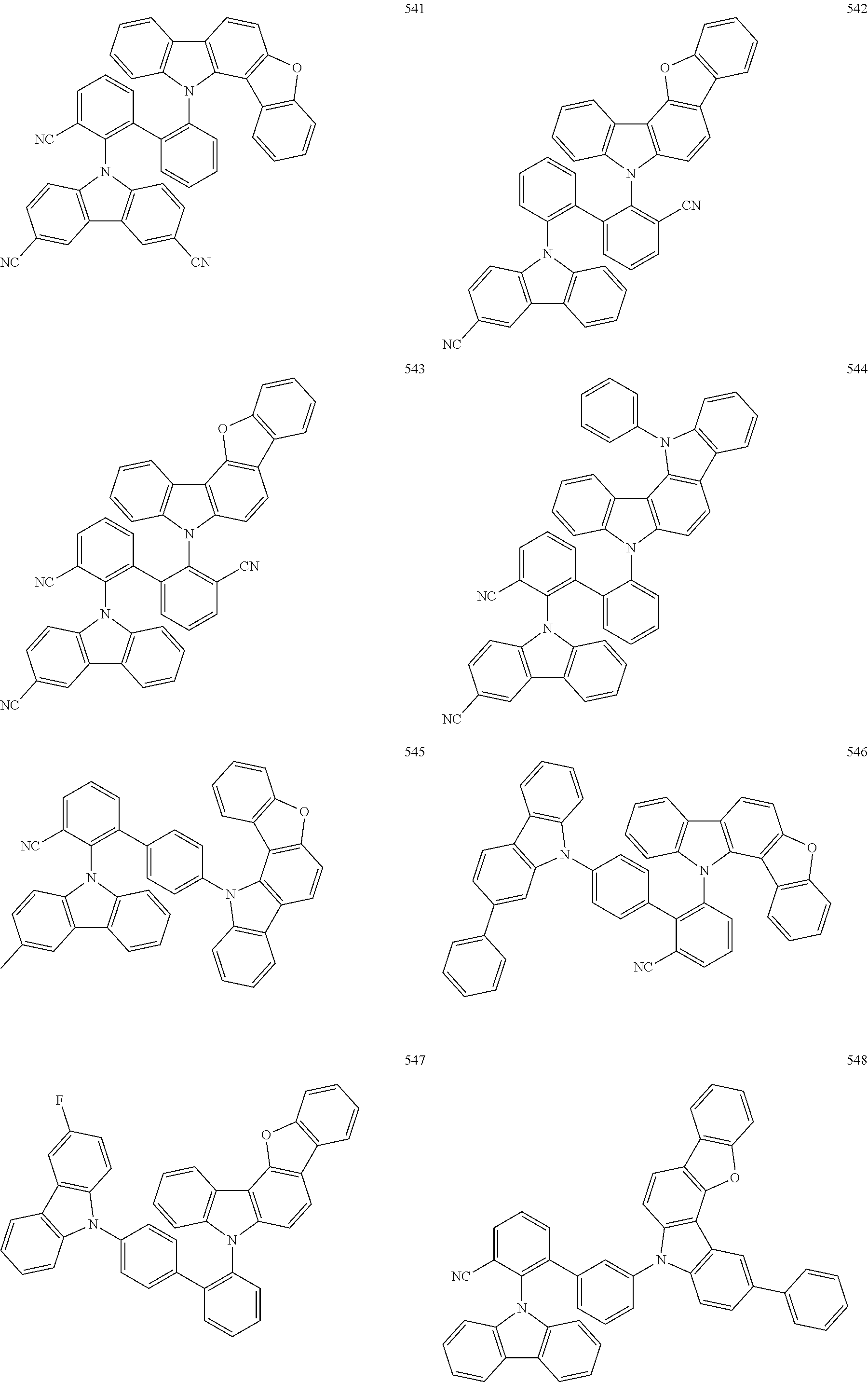

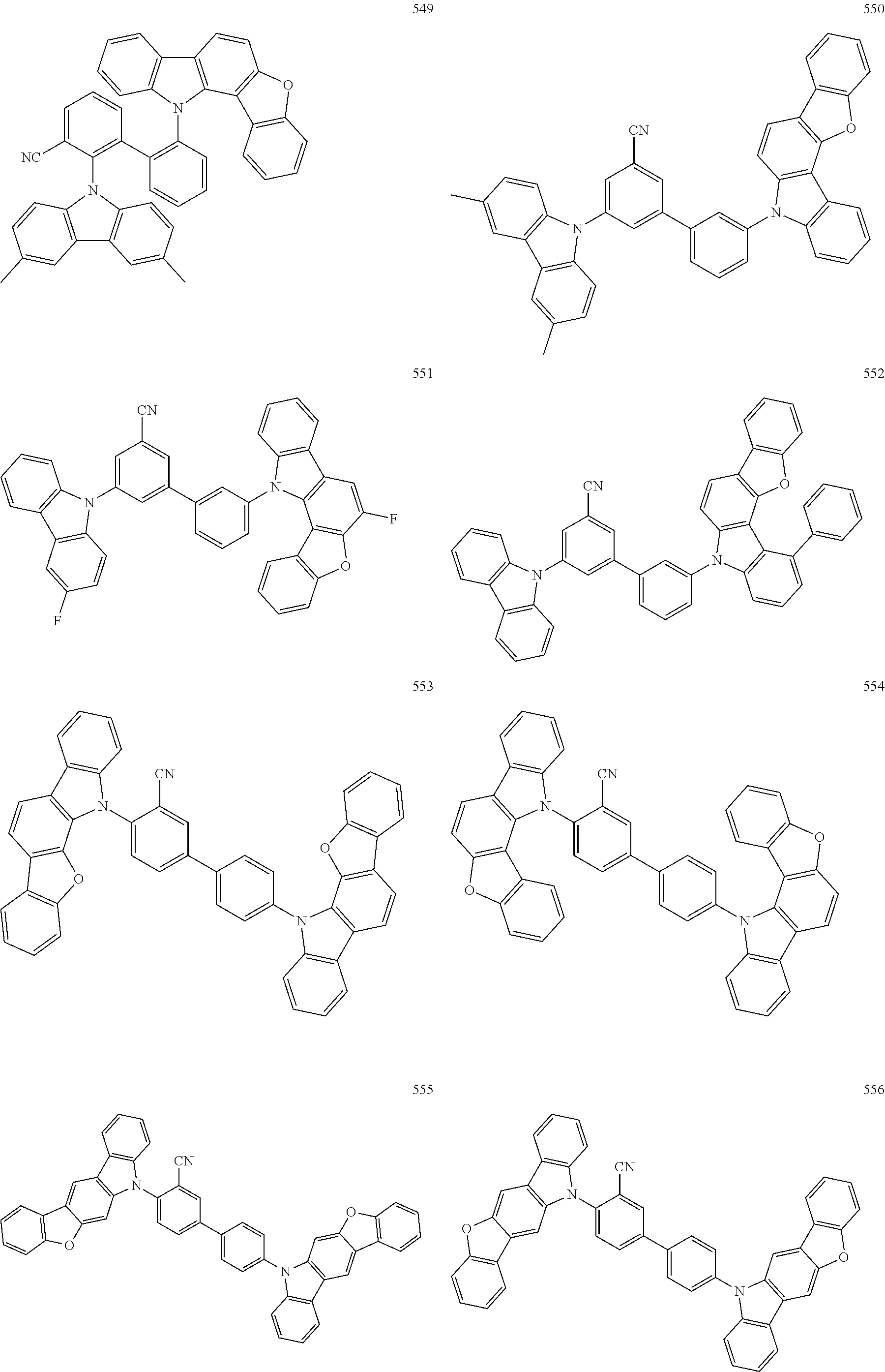

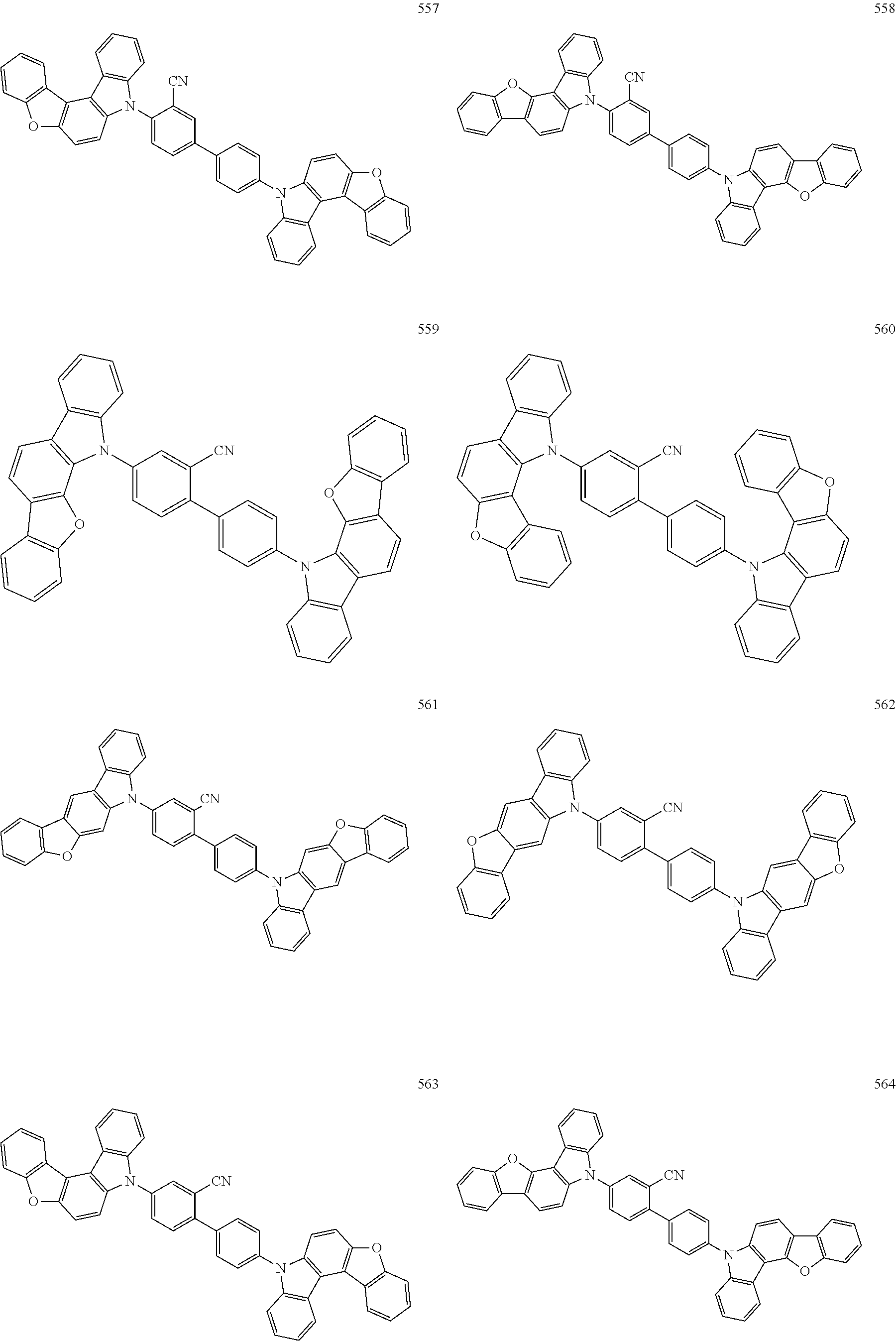

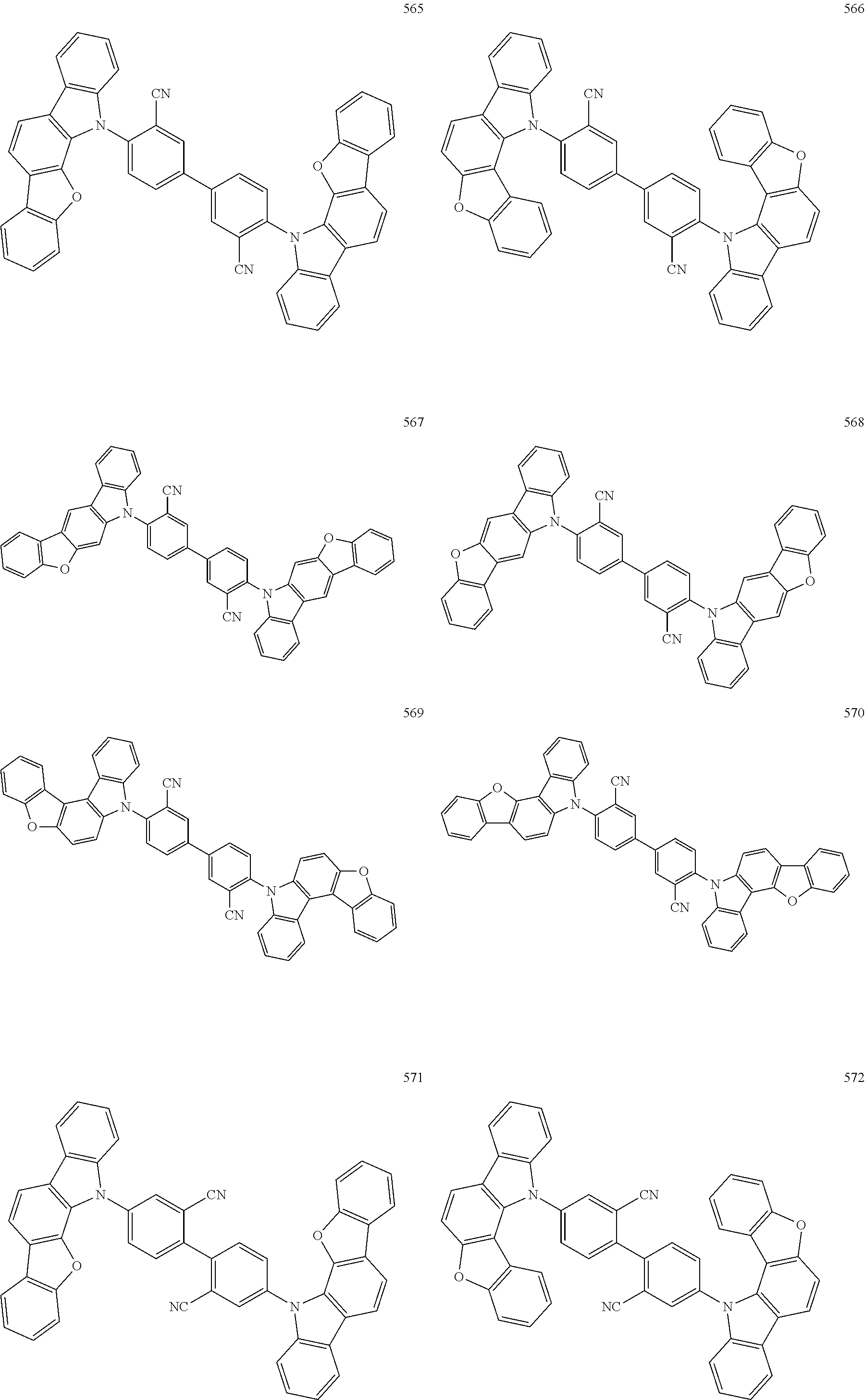

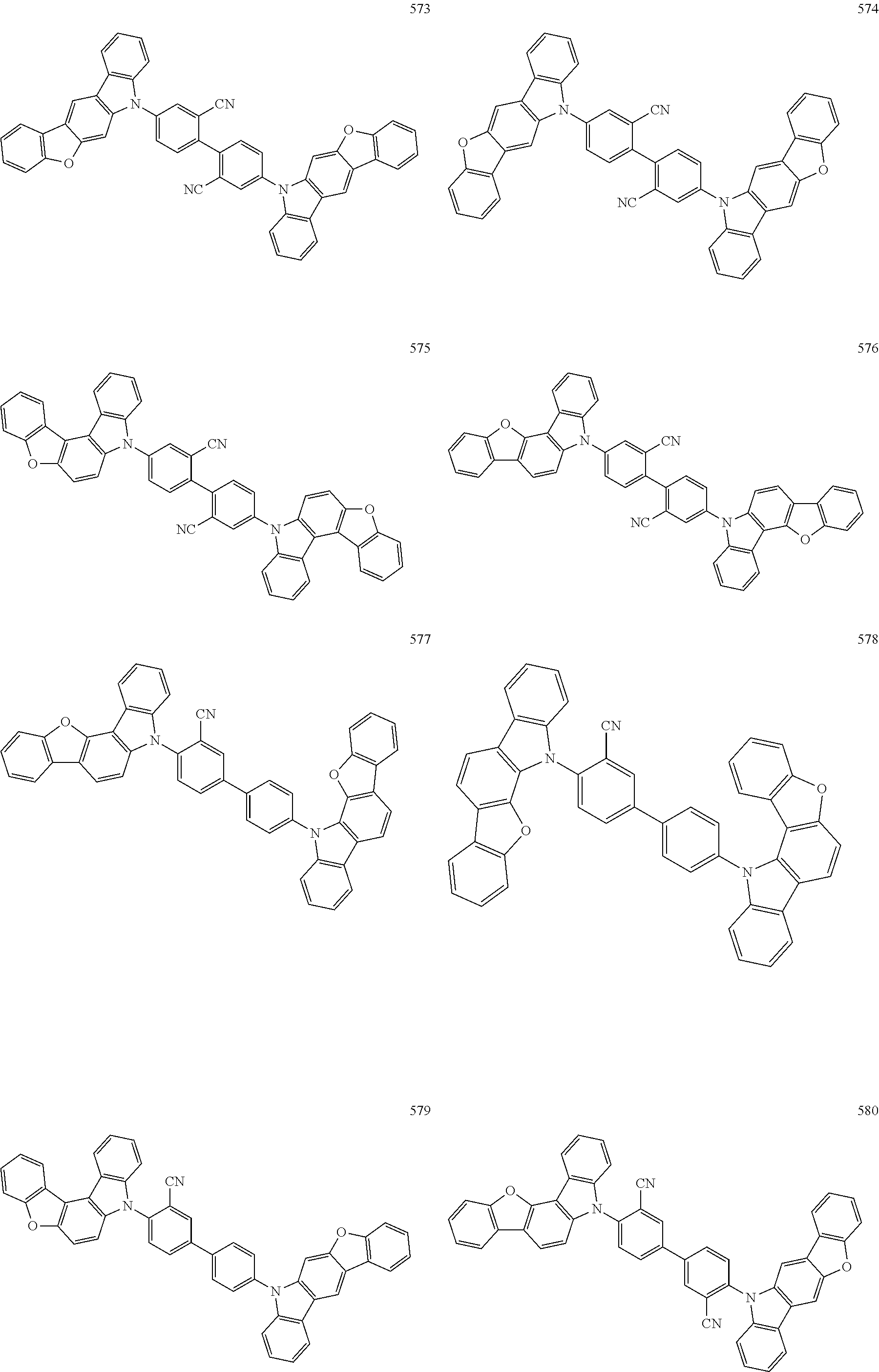

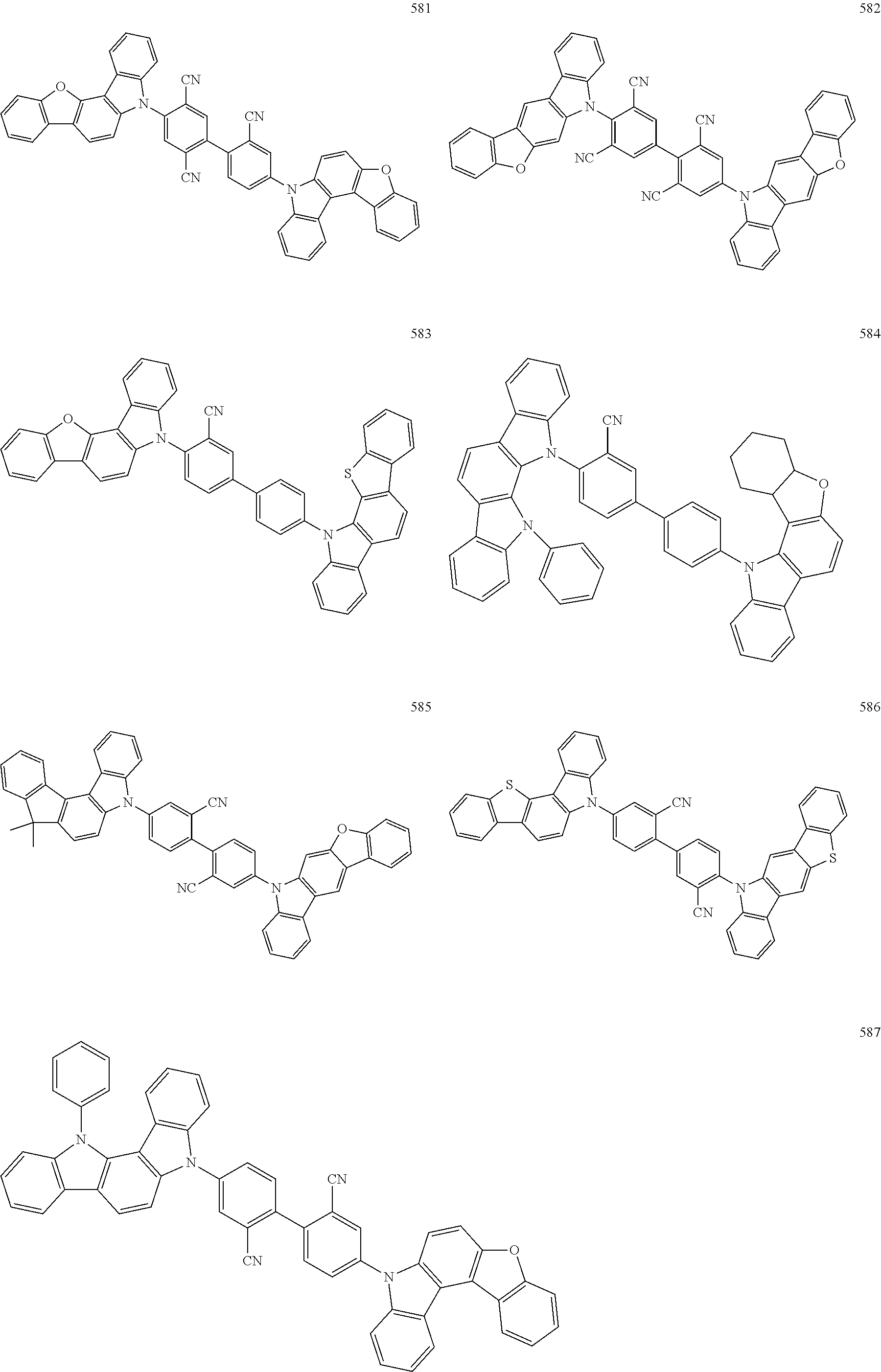

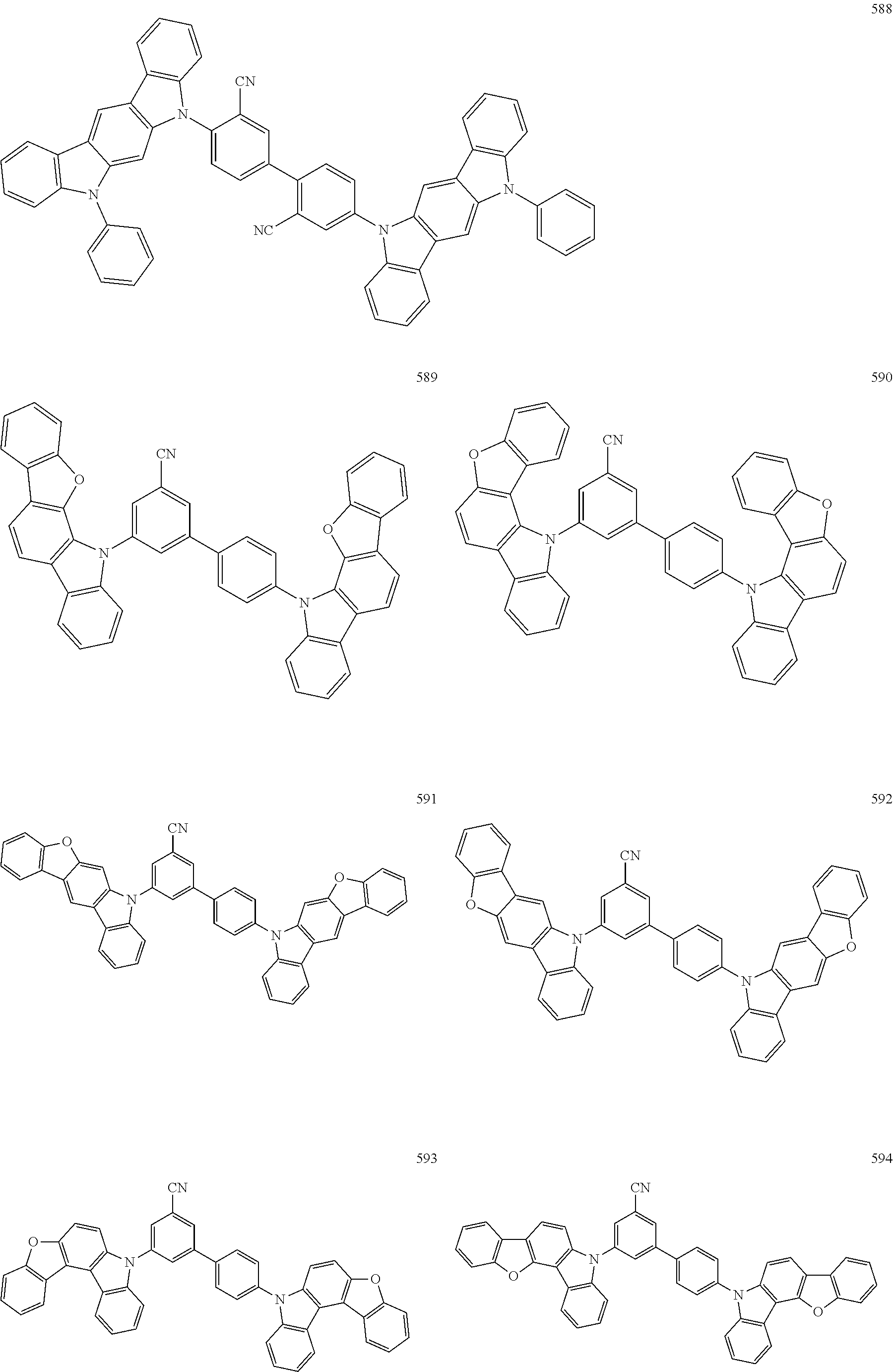

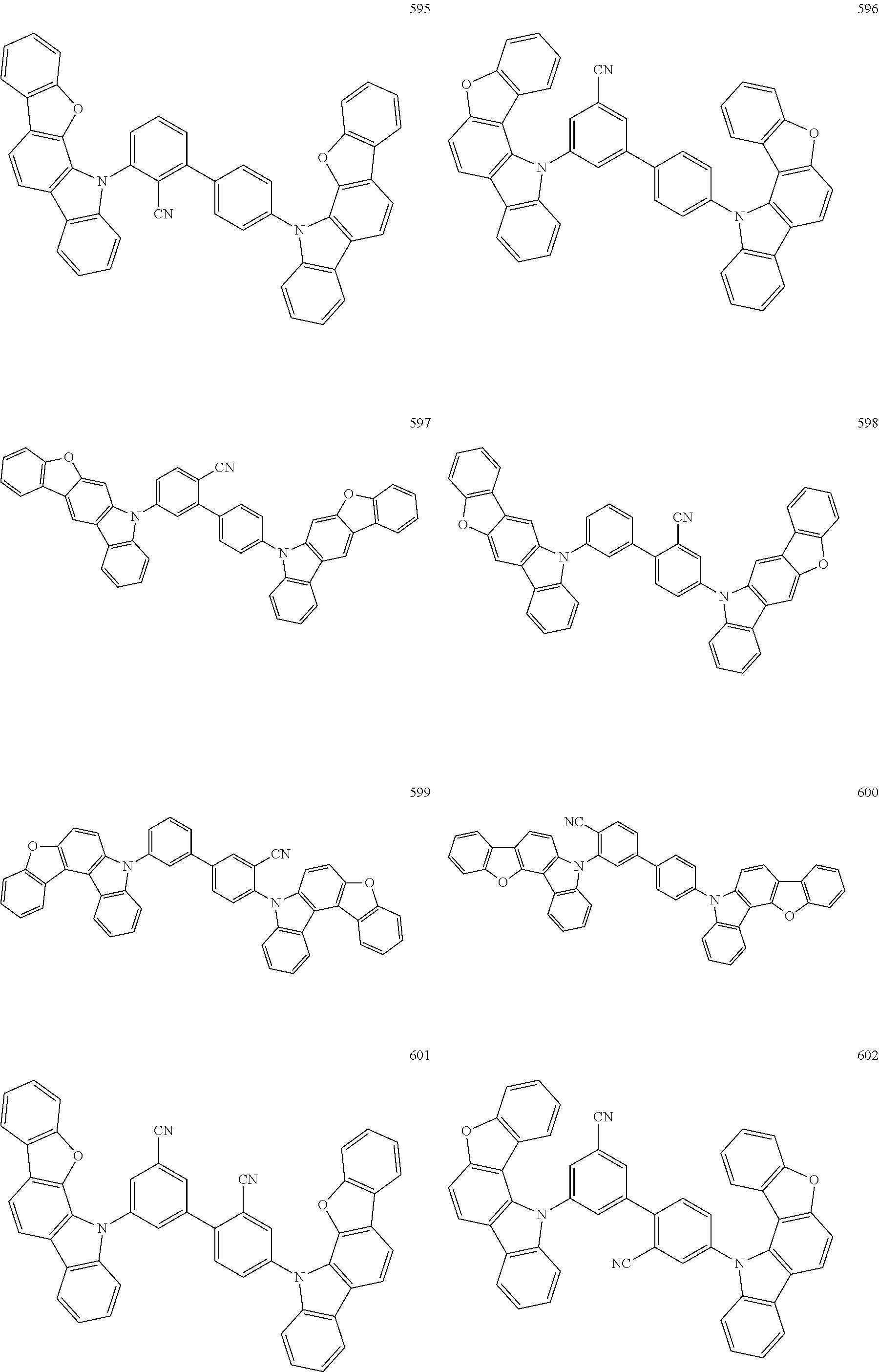

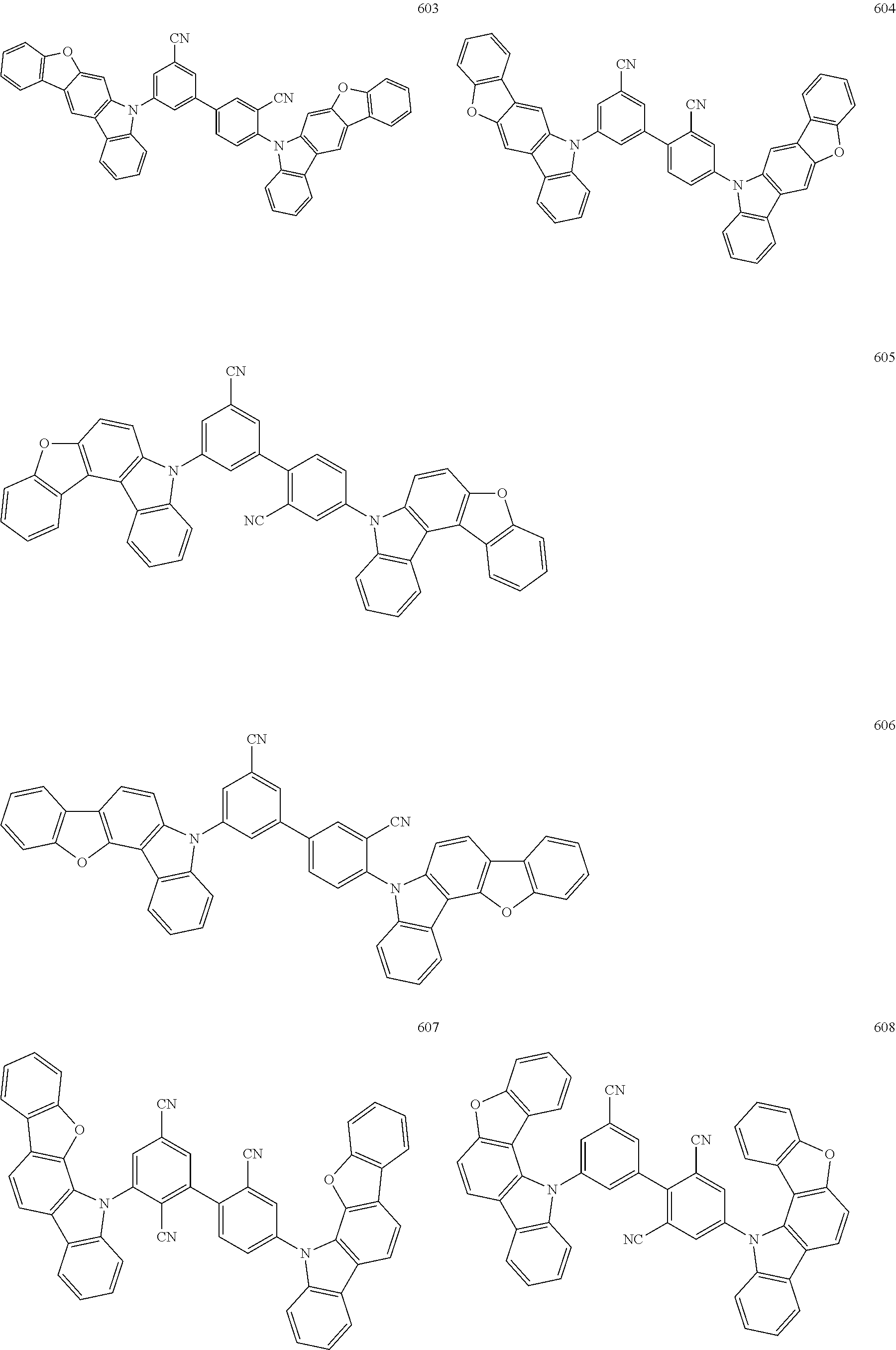

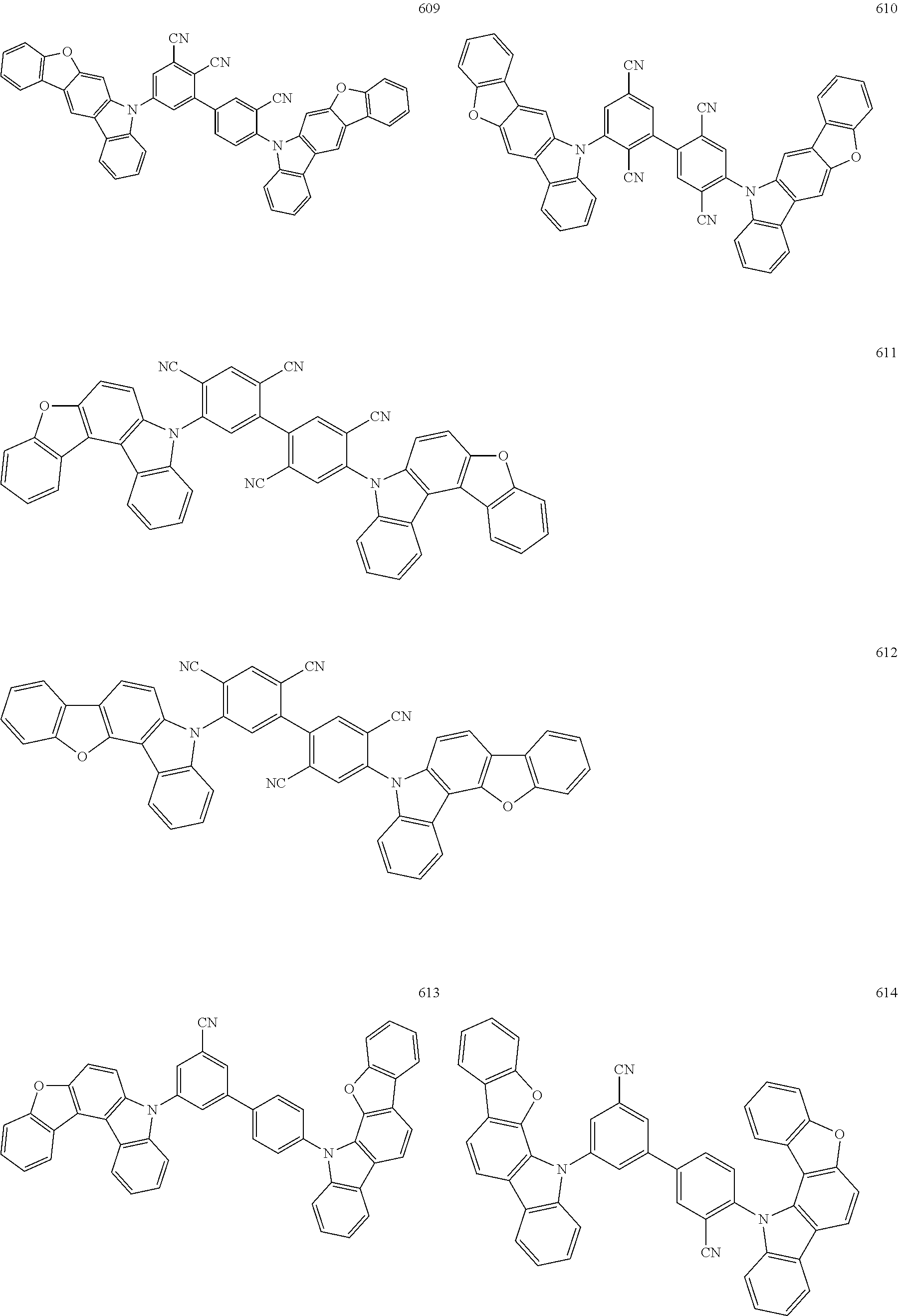

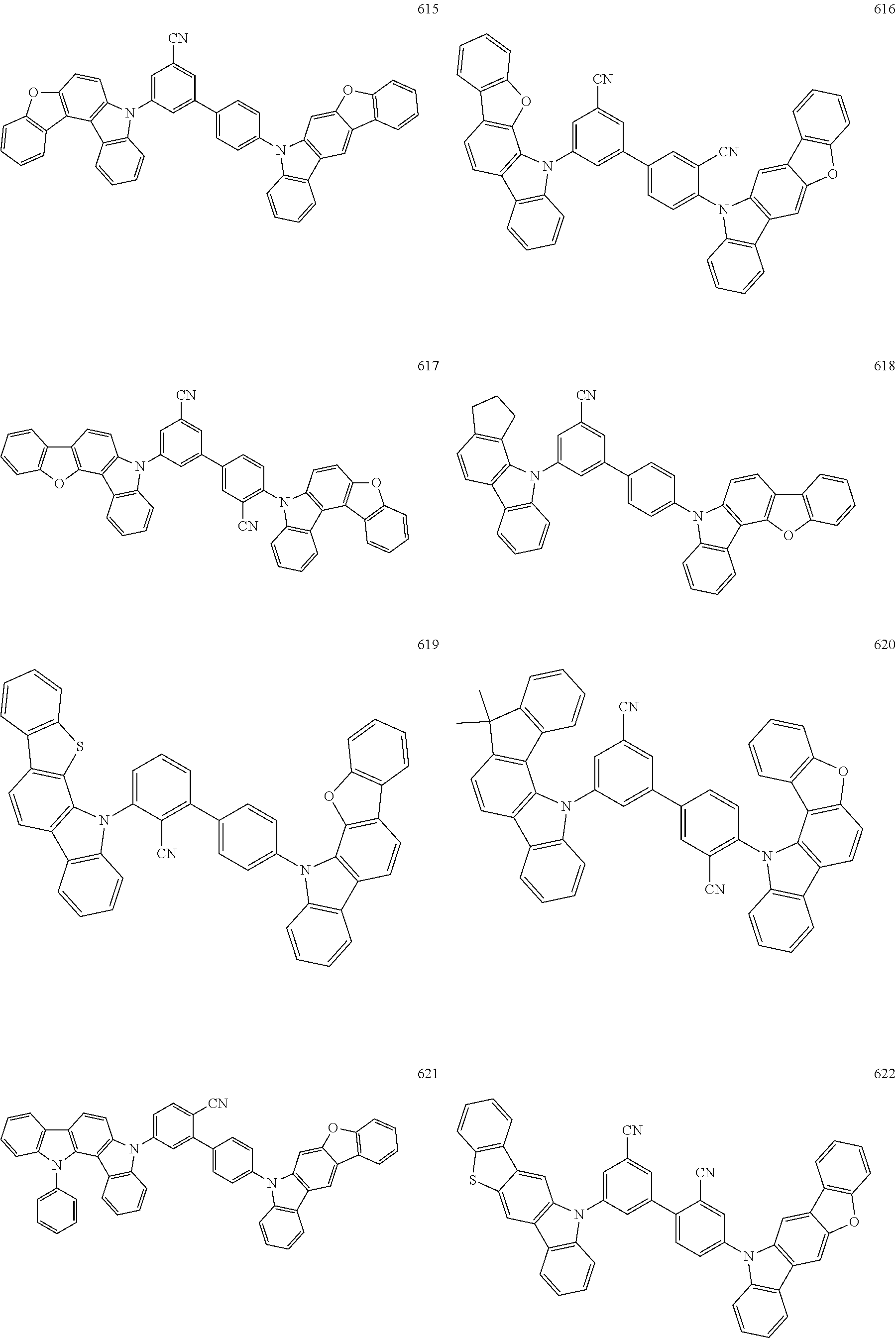

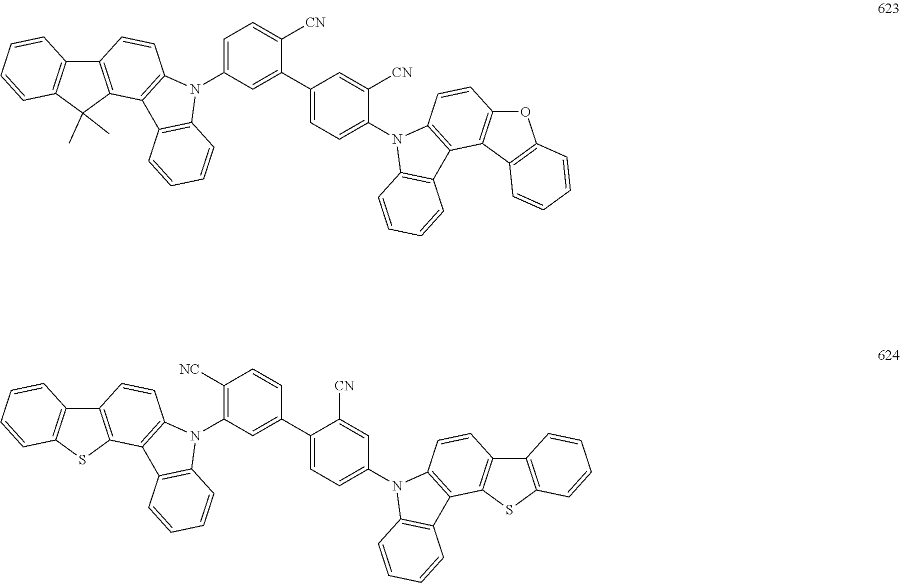 [SMILES: CC1(C)c2ccccc2-c2ccc3c(c21)c1ccccc1n3-c1ccc(C#N)c(-c2ccc(-n3c4ccccc4c4c5c(ccc43)oc3ccccc35)c(C#N)c2)c1 ; N#Cc1cc(-n2c3ccccc3c3c4sc5ccccc5c4ccc32)ccc1-c1ccc(C#N)c(-n2c3ccccc3c3c4sc5ccccc5c4ccc32)c1]

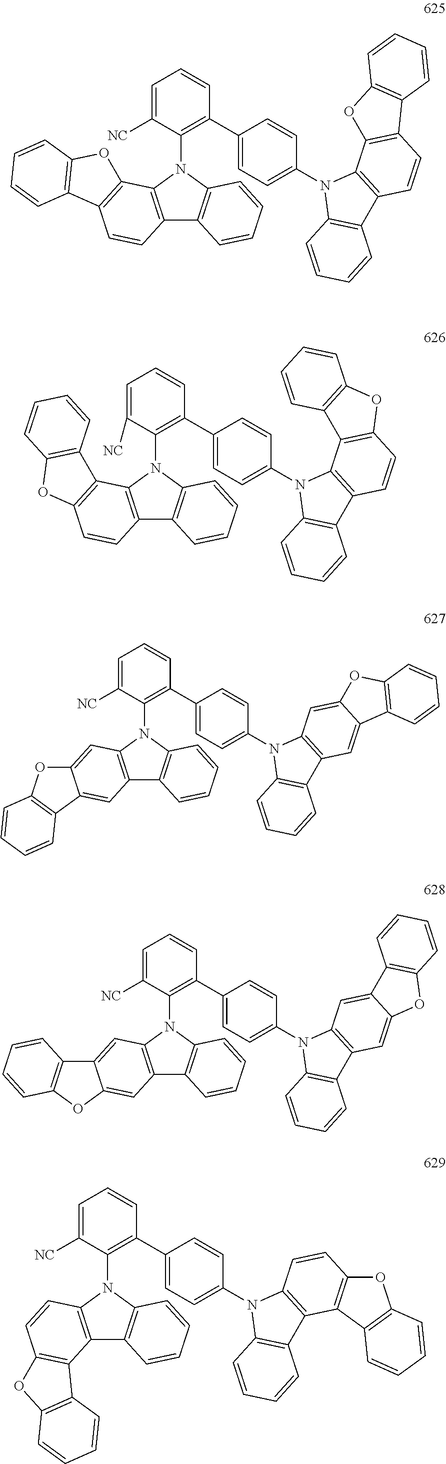 [SMILES: N#Cc1cccc(-c2ccc(-n3c4ccccc4c4ccc5c6ccccc6oc5c43)cc2)c1-n1c2ccccc2c2ccc3c4ccccc4oc3c21 ; N#Cc1cccc(-c2ccc(-n3c4ccccc4c4ccc5oc6ccccc6c5c43)cc2)c1-n1c2ccccc2c2ccc3oc4ccccc4c3c21 ; N#Cc1cccc(-c2ccc(-n3c4ccccc4c4cc5c(cc43)oc3ccccc35)cc2)c1-n1c2ccccc2c2cc3c(cc21)oc1ccccc13 ; N#Cc1cccc(-c2ccc(-n3c4ccccc4c4cc5oc6ccccc6c5cc43)cc2)c1-n1c2ccccc2c2cc3oc4ccccc4c3cc21 ; N#Cc1cccc(-c2ccc(-n3c4ccccc4c4c5c(ccc43)oc3ccccc35)cc2)c1-n1c2ccccc2c2c3c(ccc21)oc1ccccc13]

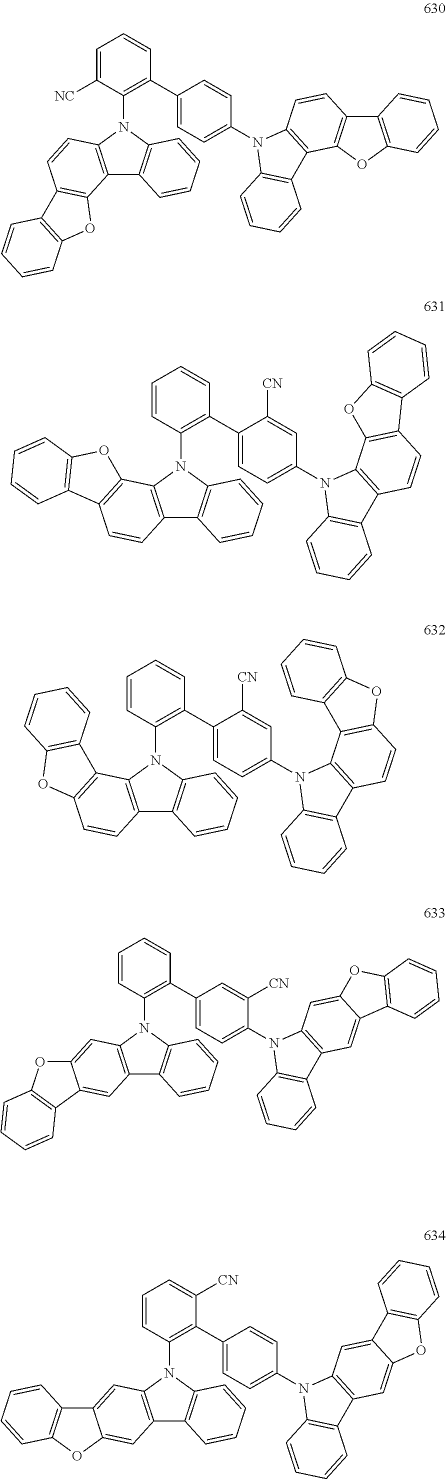 [SMILES: N#Cc1cccc(-c2ccc(-n3c4ccccc4c4c5oc6ccccc6c5ccc43)cc2)c1-n1c2ccccc2c2c3oc4ccccc4c3ccc21 ; N#Cc1cc(-n2c3ccccc3c3ccc4c5ccccc5oc4c32)ccc1-c1ccccc1-n1c2ccccc2c2ccc3c4ccccc4oc3c21 ; N#Cc1cc(-n2c3ccccc3c3ccc4oc5ccccc5c4c32)ccc1-c1ccccc1-n1c2ccccc2c2ccc3oc4ccccc4c3c21 ; N#Cc1cc(-c2ccccc2-n2c3ccccc3c3cc4c(cc32)oc2ccccc24)ccc1-n1c2ccccc2c2cc3c(cc21)oc1ccccc13 ; N#Cc1cccc(-n2c3ccccc3c3cc4oc5ccccc5c4cc32)c1-c1ccc(-n2c3ccccc3c3cc4oc5ccccc5c4cc32)cc1]

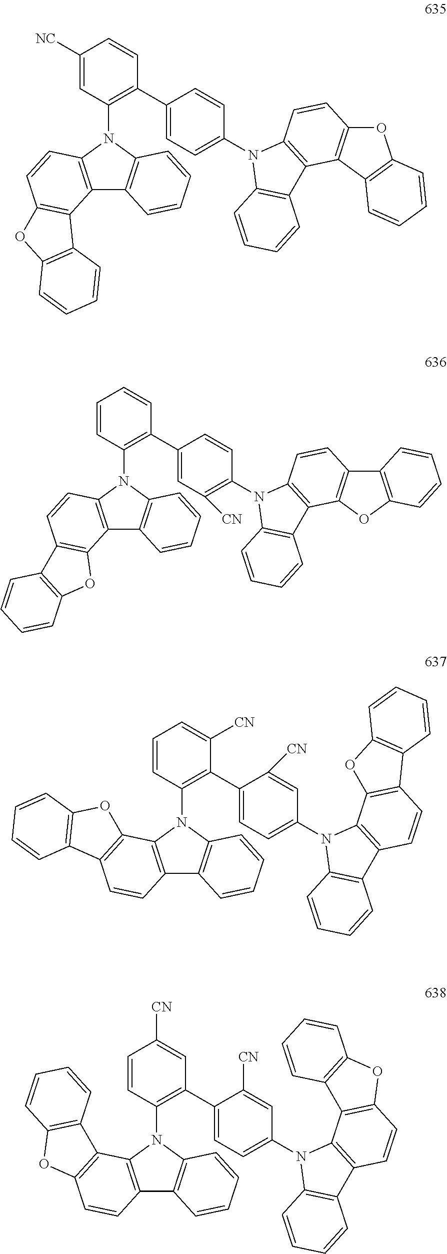 [SMILES: N#Cc1ccc(-c2ccc(-n3c4ccccc4c4c5c(ccc43)oc3ccccc35)cc2)c(-n2c3ccccc3c3c4c(ccc32)oc2ccccc24)c1 ; N#Cc1cc(-c2ccccc2-n2c3ccccc3c3c4oc5ccccc5c4ccc32)ccc1-n1c2ccccc2c2c3oc4ccccc4c3ccc21 ; N#Cc1cc(-n2c3ccccc3c3ccc4c5ccccc5oc4c32)ccc1-c1c(C#N)cccc1-n1c2ccccc2c2ccc3c4ccccc4oc3c21 ; N#Cc1ccc(-n2c3ccccc3c3ccc4oc5ccccc5c4c32)c(-c2ccc(-n3c4ccccc4c4ccc5oc6ccccc6c5c43)cc2C#N)c1]

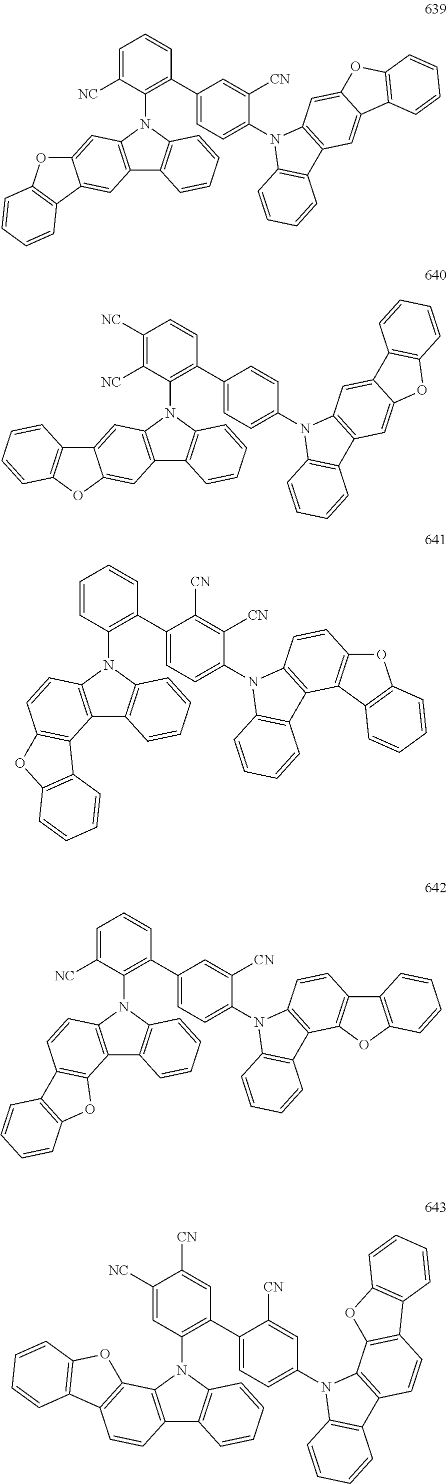 [SMILES: N#Cc1cc(-c2cccc(C#N)c2-n2c3ccccc3c3cc4c(cc32)oc2ccccc24)ccc1-n1c2ccccc2c2cc3c(cc21)oc1ccccc13 ; N#Cc1ccc(-c2ccc(-n3c4ccccc4c4cc5oc6ccccc6c5cc43)cc2)c(-n2c3ccccc3c3cc4oc5ccccc5c4cc32)c1C#N ; N#Cc1c(-c2ccccc2-n2c3ccccc3c3c4c(ccc32)oc2ccccc24)ccc(-n2c3ccccc3c3c4c(ccc32)oc2ccccc24)c1C#N ; N#Cc1cc(-c2cccc(C#N)c2-n2c3ccccc3c3c4oc5ccccc5c4ccc32)ccc1-n1c2ccccc2c2c3oc4ccccc4c3ccc21 ; N#Cc1cc(-c2ccc(-n3c4ccccc4c4ccc5c6ccccc6oc5c43)cc2C#N)c(-n2c3ccccc3c3ccc4c5ccccc5oc4c32)cc1C#N]

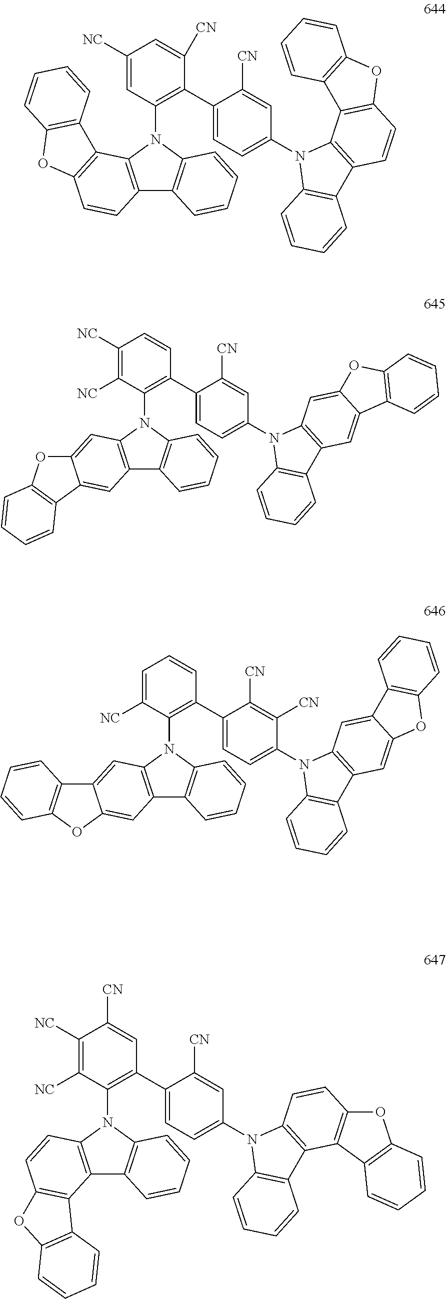 [SMILES: N#Cc1cc(C#N)c(-c2ccc(-n3c4ccccc4c4ccc5oc6ccccc6c5c43)cc2C#N)c(-n2c3ccccc3c3ccc4oc5ccccc5c4c32)c1 ; N#Cc1cc(-n2c3ccccc3c3cc4c(cc32)oc2ccccc24)ccc1-c1ccc(C#N)c(C#N)c1-n1c2ccccc2c2cc3c(cc21)oc1ccccc13 ; N#Cc1cccc(-c2ccc(-n3c4ccccc4c4cc5oc6ccccc6c5cc43)c(C#N)c2C#N)c1-n1c2ccccc2c2cc3oc4ccccc4c3cc21 ; N#Cc1cc(-n2c3ccccc3c3c4c(ccc32)oc2ccccc24)ccc1-c1cc(C#N)c(C#N)c(C#N)c1-n1c2ccccc2c2c3c(ccc21)oc1ccccc13]

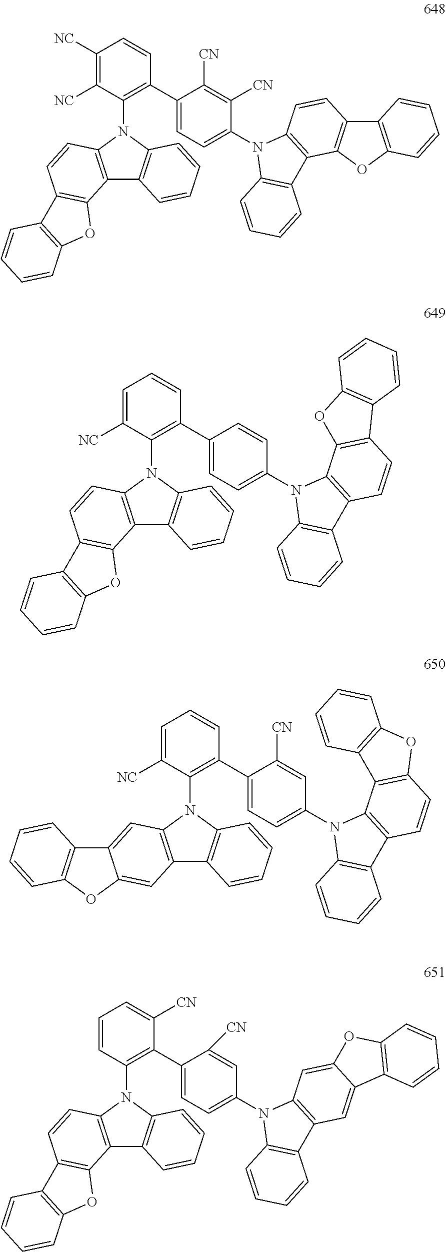 [SMILES: N#Cc1ccc(-c2ccc(-n3c4ccccc4c4c5oc6ccccc6c5ccc43)c(C#N)c2C#N)c(-n2c3ccccc3c3c4oc5ccccc5c4ccc32)c1C#N ; N#Cc1cccc(-c2ccc(-n3c4ccccc4c4ccc5c6ccccc6oc5c43)cc2)c1-n1c2ccccc2c2c3oc4ccccc4c3ccc21 ; N#Cc1cc(-n2c3ccccc3c3ccc4oc5ccccc5c4c32)ccc1-c1cccc(C#N)c1-n1c2ccccc2c2cc3oc4ccccc4c3cc21 ; N#Cc1cc(-n2c3ccccc3c3cc4c(cc32)oc2ccccc24)ccc1-c1c(C#N)cccc1-n1c2ccccc2c2c3oc4ccccc4c3ccc21]

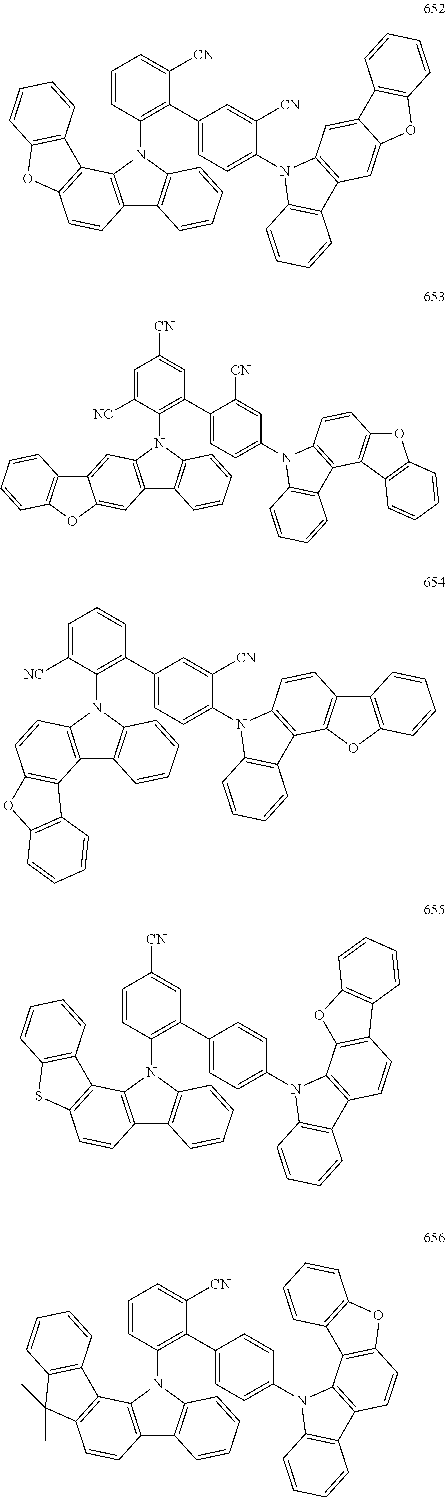 [SMILES: N#Cc1cc(-c2c(C#N)cccc2-n2c3ccccc3c3ccc4oc5ccccc5c4c32)ccc1-n1c2ccccc2c2cc3oc4ccccc4c3cc21 ; N#Cc1cc(C#N)c(-n2c3ccccc3c3cc4oc5ccccc5c4cc32)c(-c2ccc(-n3c4ccccc4c4c5c(ccc43)oc3ccccc35)cc2C#N)c1 ; N#Cc1cc(-c2cccc(C#N)c2-n2c3ccccc3c3c4c(ccc32)oc2ccccc24)ccc1-n1c2ccccc2c2c3oc4ccccc4c3ccc21 ; N#Cc1ccc(-n2c3ccccc3c3ccc4sc5ccccc5c4c32)c(-c2ccc(-n3c4ccccc4c4ccc5c6ccccc6oc5c43)cc2)c1 ; CC1(C)c2ccccc2-c2c1ccc1c3ccccc3n(-c3cccc(C#N)c3-c3ccc(-n4c5ccccc5c5ccc6oc7ccccc7c6c54)cc3)c21]

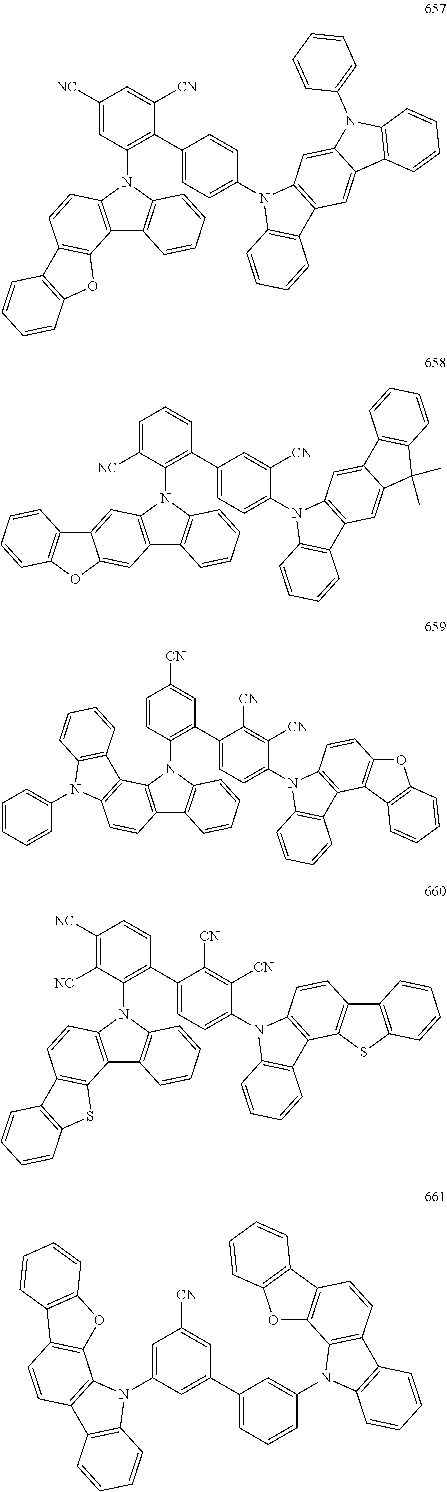

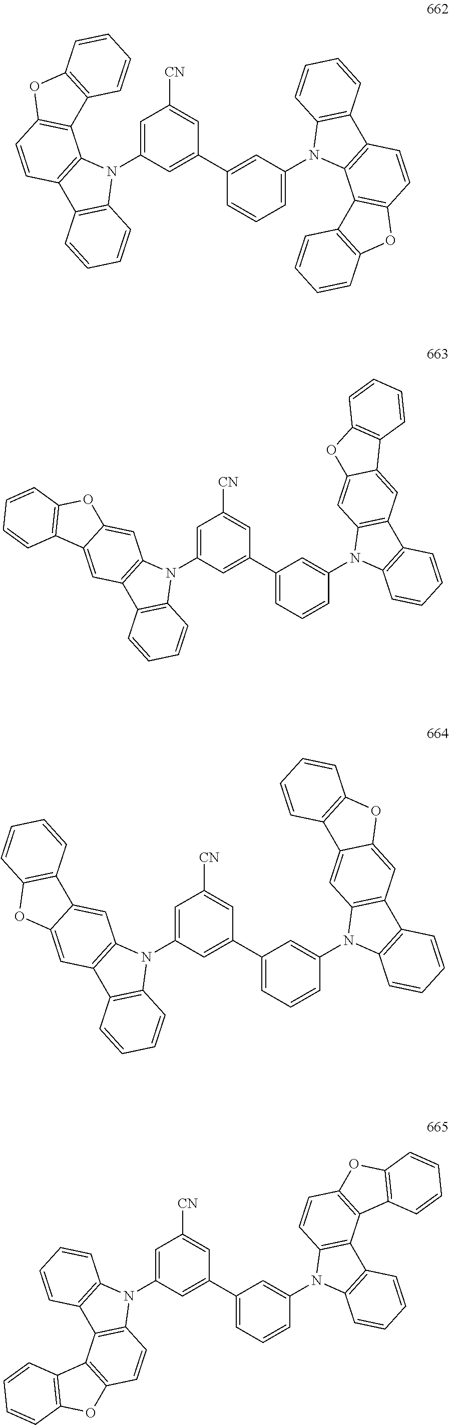 [SMILES: N#Cc1cc(-c2cccc(-n3c4ccccc4c4ccc5oc6ccccc6c5c43)c2)cc(-n2c3ccccc3c3ccc4oc5ccccc5c4c32)c1 ; N#Cc1cc(-c2cccc(-n3c4ccccc4c4cc5c(cc43)oc3ccccc35)c2)cc(-n2c3ccccc3c3cc4c(cc32)oc2ccccc24)c1 ; N#Cc1cc(-c2cccc(-n3c4ccccc4c4cc5oc6ccccc6c5cc43)c2)cc(-n2c3ccccc3c3cc4oc5ccccc5c4cc32)c1 ; N#Cc1cc(-c2cccc(-n3c4ccccc4c4c5c(ccc43)oc3ccccc35)c2)cc(-n2c3ccccc3c3c4c(ccc32)oc2ccccc24)c1]

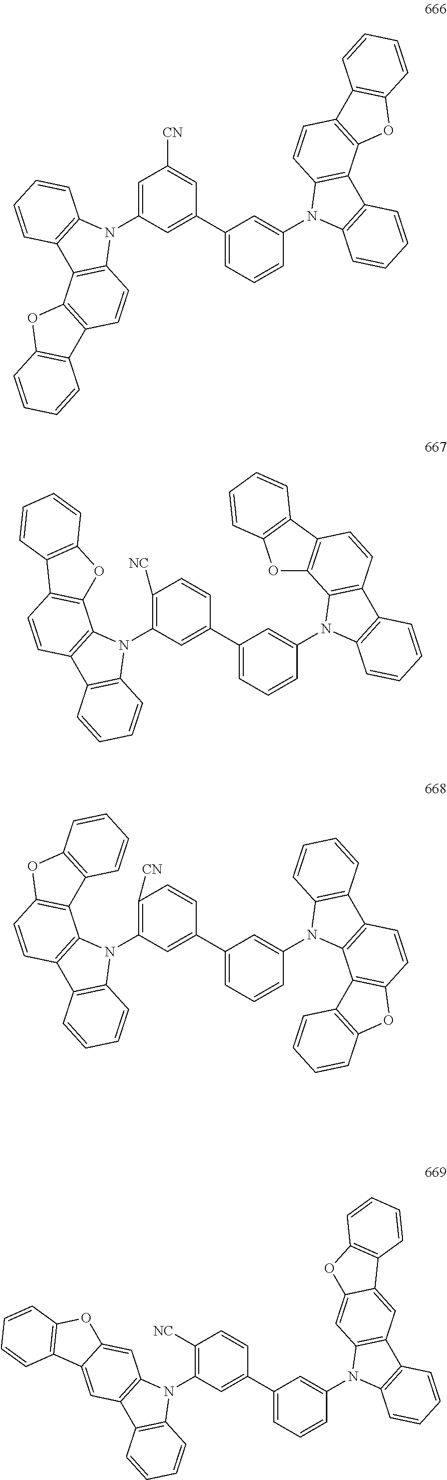 [SMILES: N#Cc1cc(-c2cccc(-n3c4ccccc4c4c5oc6ccccc6c5ccc43)c2)cc(-n2c3ccccc3c3c4oc5ccccc5c4ccc32)c1 ; N#Cc1ccc(-c2cccc(-n3c4ccccc4c4ccc5c6ccccc6oc5c43)c2)cc1-n1c2ccccc2c2ccc3c4ccccc4oc3c21 ; N#Cc1ccc(-c2cccc(-n3c4ccccc4c4ccc5oc6ccccc6c5c43)c2)cc1-n1c2ccccc2c2ccc3oc4ccccc4c3c21 ; N#Cc1ccc(-c2cccc(-n3c4ccccc4c4cc5c(cc43)oc3ccccc35)c2)cc1-n1c2ccccc2c2cc3c(cc21)oc1ccccc13]

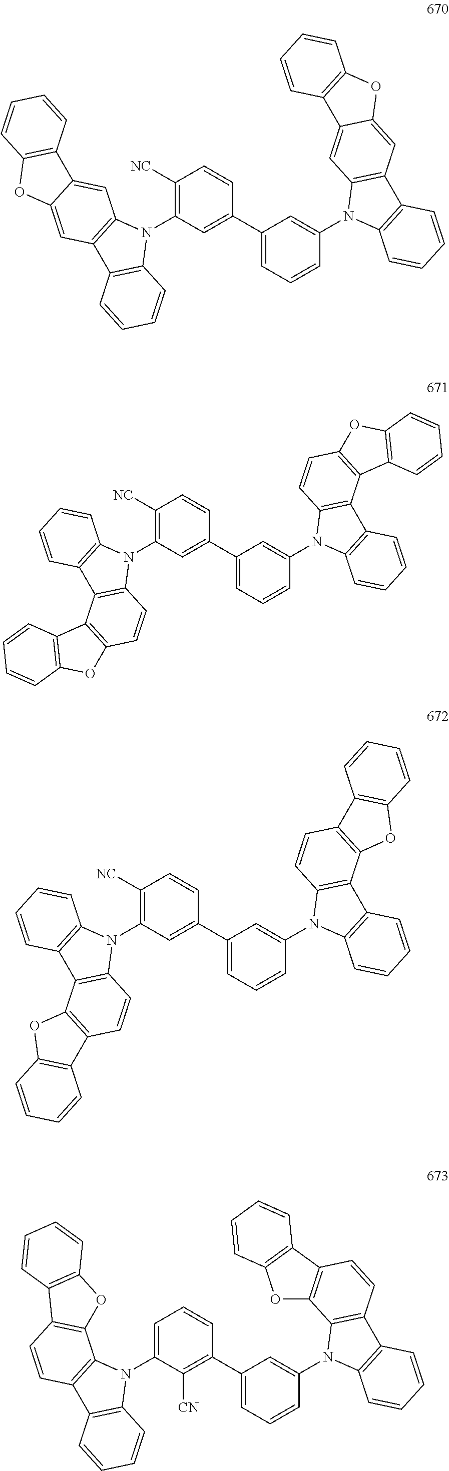 [SMILES: N#Cc1ccc(-c2cccc(-n3c4ccccc4c4cc5oc6ccccc6c5cc43)c2)cc1-n1c2ccccc2c2cc3oc4ccccc4c3cc21 ; N#Cc1ccc(-c2cccc(-n3c4ccccc4c4c5c(ccc43)oc3ccccc35)c2)cc1-n1c2ccccc2c2c3c(ccc21)oc1ccccc13 ; N#Cc1ccc(-c2cccc(-n3c4ccccc4c4c5oc6ccccc6c5ccc43)c2)cc1-n1c2ccccc2c2c3oc4ccccc4c3ccc21 ; N#Cc1c(-c2cccc(-n3c4ccccc4c4ccc5c6ccccc6oc5c43)c2)cccc1-n1c2ccccc2c2ccc3c4ccccc4oc3c21]

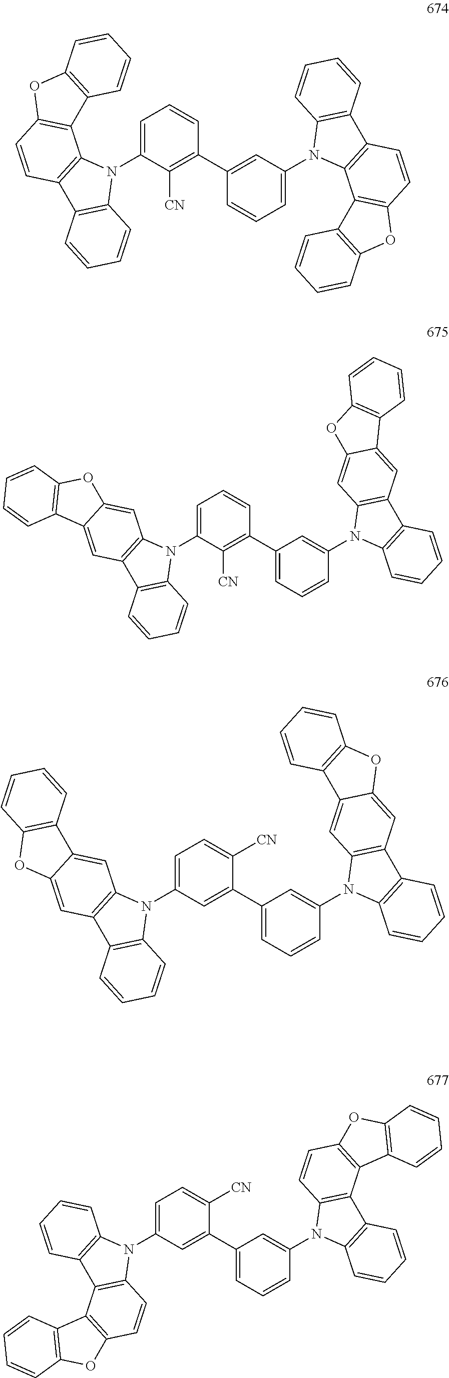 [SMILES: N#Cc1c(-c2cccc(-n3c4ccccc4c4ccc5oc6ccccc6c5c43)c2)cccc1-n1c2ccccc2c2ccc3oc4ccccc4c3c21 ; N#Cc1c(-c2cccc(-n3c4ccccc4c4cc5c(cc43)oc3ccccc35)c2)cccc1-n1c2ccccc2c2cc3c(cc21)oc1ccccc13 ; N#Cc1ccc(-n2c3ccccc3c3cc4oc5ccccc5c4cc32)cc1-c1cccc(-n2c3ccccc3c3cc4oc5ccccc5c4cc32)c1 ; N#Cc1ccc(-n2c3ccccc3c3c4c(ccc32)oc2ccccc24)cc1-c1cccc(-n2c3ccccc3c3c4c(ccc32)oc2ccccc24)c1]

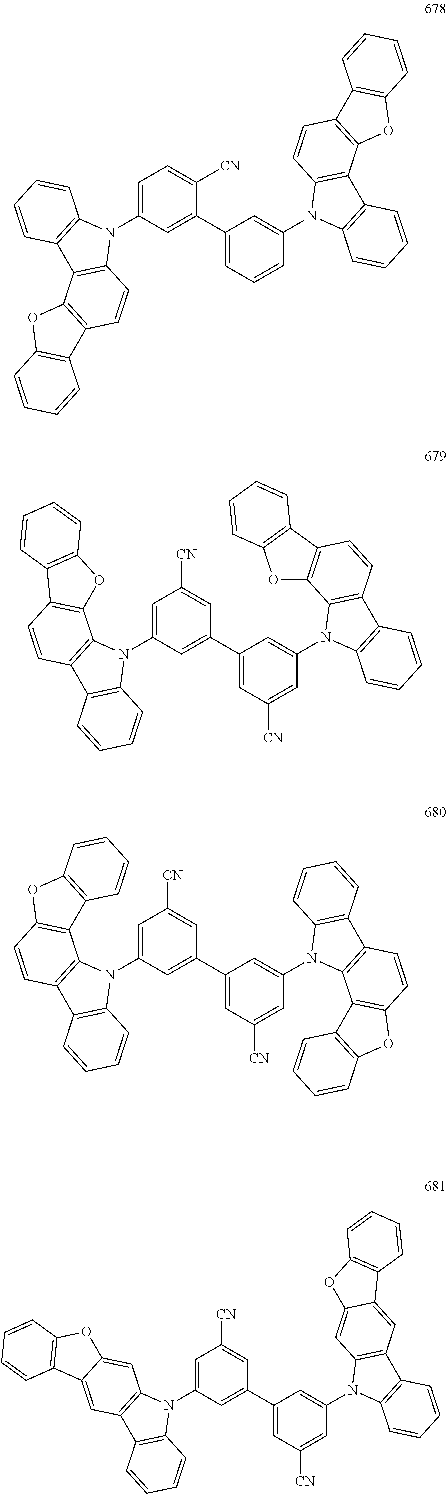 [SMILES: N#Cc1ccc(-n2c3ccccc3c3c4oc5ccccc5c4ccc32)cc1-c1cccc(-n2c3ccccc3c3c4oc5ccccc5c4ccc32)c1 ; N#Cc1cc(-c2cc(C#N)cc(-n3c4ccccc4c4ccc5c6ccccc6oc5c43)c2)cc(-n2c3ccccc3c3ccc4c5ccccc5oc4c32)c1 ; N#Cc1cc(-c2cc(C#N)cc(-n3c4ccccc4c4ccc5oc6ccccc6c5c43)c2)cc(-n2c3ccccc3c3ccc4oc5ccccc5c4c32)c1 ; N#Cc1cc(-c2cc(C#N)cc(-n3c4ccccc4c4cc5c(cc43)oc3ccccc35)c2)cc(-n2c3ccccc3c3cc4c(cc32)oc2ccccc24)c1]

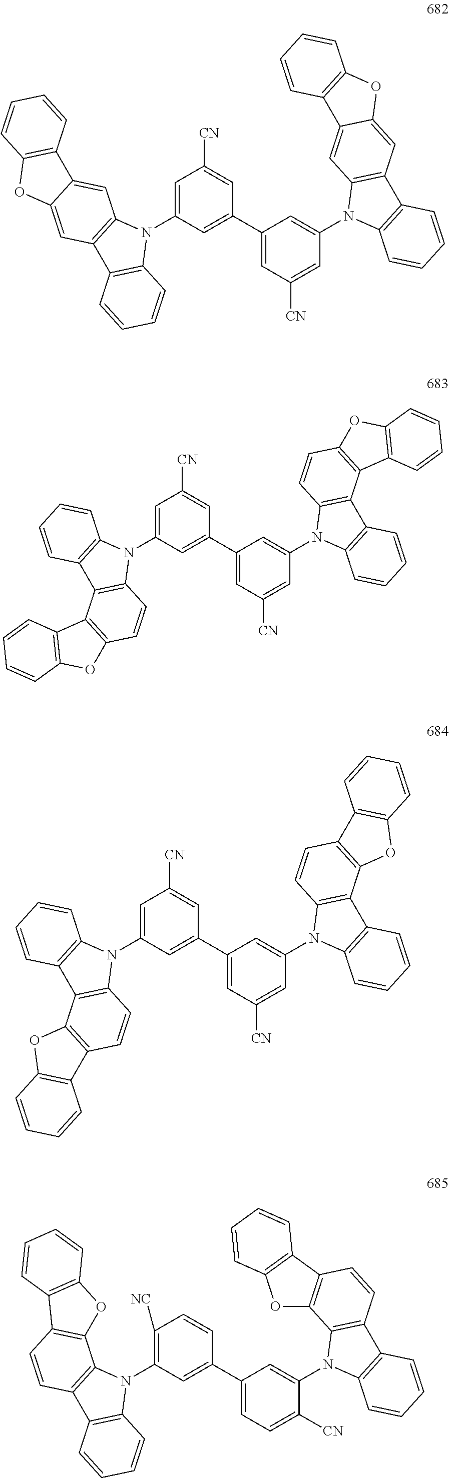 [SMILES: N#Cc1cc(-c2cc(C#N)cc(-n3c4ccccc4c4cc5oc6ccccc6c5cc43)c2)cc(-n2c3ccccc3c3cc4oc5ccccc5c4cc32)c1 ; N#Cc1cc(-c2cc(C#N)cc(-n3c4ccccc4c4c5c(ccc43)oc3ccccc35)c2)cc(-n2c3ccccc3c3c4c(ccc32)oc2ccccc24)c1 ; N#Cc1cc(-c2cc(C#N)cc(-n3c4ccccc4c4c5oc6ccccc6c5ccc43)c2)cc(-n2c3ccccc3c3c4oc5ccccc5c4ccc32)c1 ; N#Cc1ccc(-c2ccc(C#N)c(-n3c4ccccc4c4ccc5c6ccccc6oc5c43)c2)cc1-n1c2ccccc2c2ccc3c4ccccc4oc3c21]

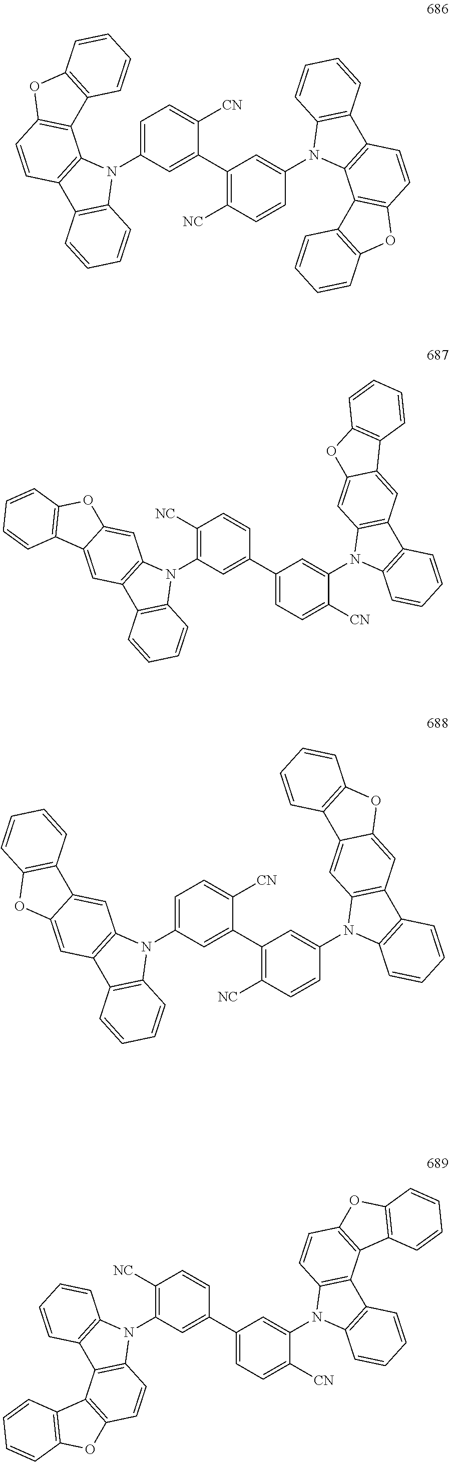 [SMILES: N#Cc1ccc(-n2c3ccccc3c3ccc4oc5ccccc5c4c32)cc1-c1cc(-n2c3ccccc3c3ccc4oc5ccccc5c4c32)ccc1C#N ; N#Cc1ccc(-c2ccc(C#N)c(-n3c4ccccc4c4cc5c(cc43)oc3ccccc35)c2)cc1-n1c2ccccc2c2cc3c(cc21)oc1ccccc13 ; N#Cc1ccc(-n2c3ccccc3c3cc4oc5ccccc5c4cc32)cc1-c1cc(-n2c3ccccc3c3cc4oc5ccccc5c4cc32)ccc1C#N ; N#Cc1ccc(-c2ccc(C#N)c(-n3c4ccccc4c4c5c(ccc43)oc3ccccc35)c2)cc1-n1c2ccccc2c2c3c(ccc21)oc1ccccc13]

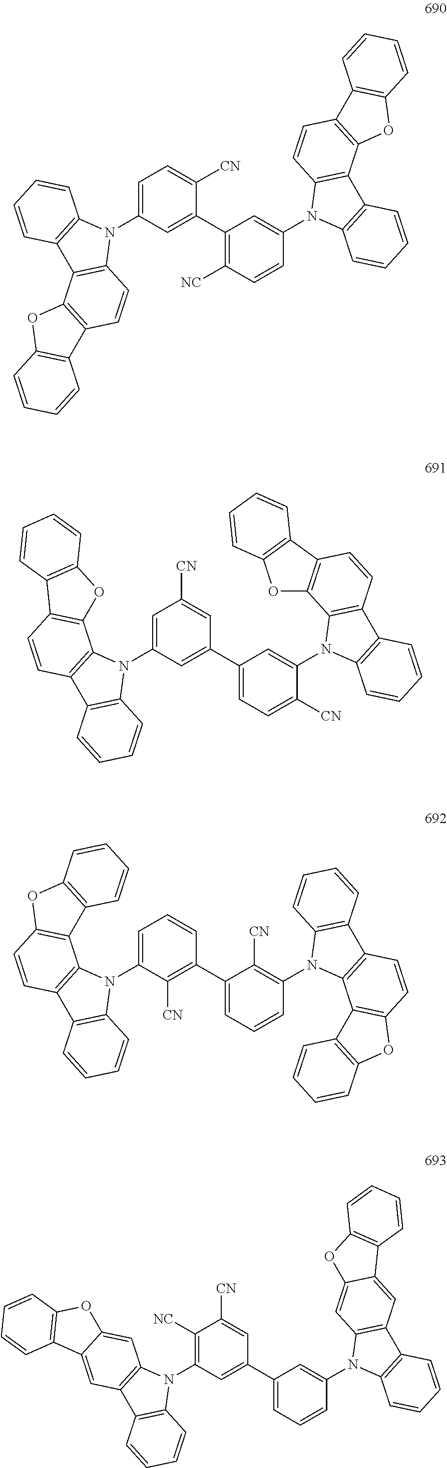 [SMILES: N#Cc1ccc(-n2c3ccccc3c3c4oc5ccccc5c4ccc32)cc1-c1cc(-n2c3ccccc3c3c4oc5ccccc5c4ccc32)ccc1C#N ; N#Cc1cc(-c2ccc(C#N)c(-n3c4ccccc4c4ccc5c6ccccc6oc5c43)c2)cc(-n2c3ccccc3c3ccc4c5ccccc5oc4c32)c1 ; N#Cc1c(-c2cccc(-n3c4ccccc4c4ccc5oc6ccccc6c5c43)c2C#N)cccc1-n1c2ccccc2c2ccc3oc4ccccc4c3c21 ; N#Cc1cc(-c2cccc(-n3c4ccccc4c4cc5c(cc43)oc3ccccc35)c2)cc(-n2c3ccccc3c3cc4c(cc32)oc2ccccc24)c1C#N]

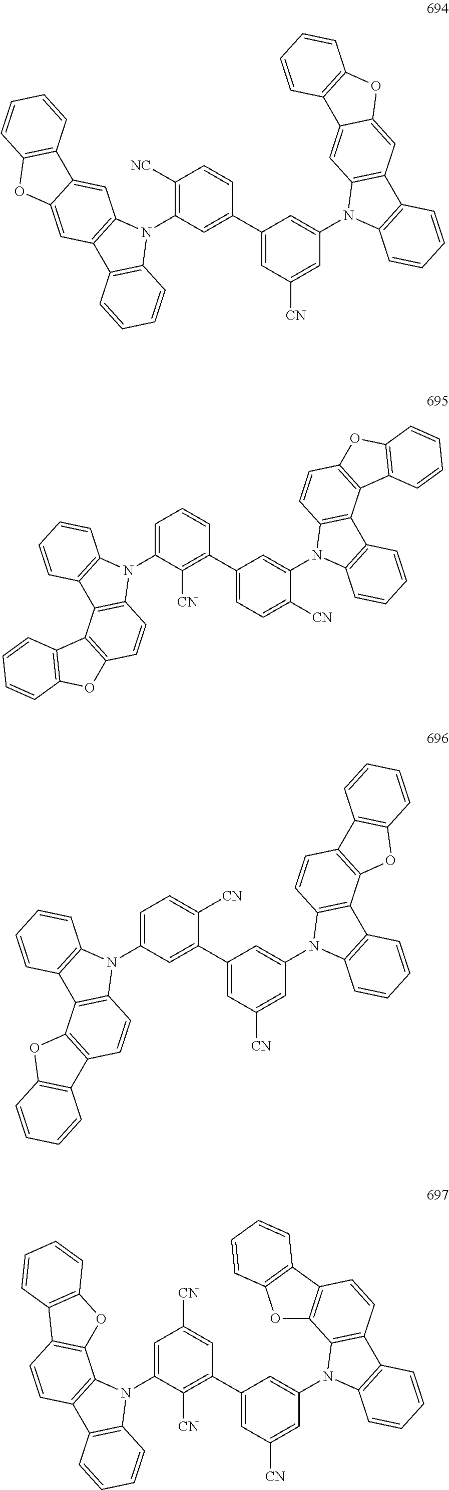 [SMILES: N#Cc1cc(-c2ccc(C#N)c(-n3c4ccccc4c4cc5oc6ccccc6c5cc43)c2)cc(-n2c3ccccc3c3cc4oc5ccccc5c4cc32)c1 ; N#Cc1ccc(-c2cccc(-n3c4ccccc4c4c5c(ccc43)oc3ccccc35)c2C#N)cc1-n1c2ccccc2c2c3c(ccc21)oc1ccccc13 ; N#Cc1cc(-c2cc(-n3c4ccccc4c4c5oc6ccccc6c5ccc43)ccc2C#N)cc(-n2c3ccccc3c3c4oc5ccccc5c4ccc32)c1 ; N#Cc1cc(-c2cc(C#N)cc(-n3c4ccccc4c4ccc5c6ccccc6oc5c43)c2C#N)cc(-n2c3ccccc3c3ccc4c5ccccc5oc4c32)c1]

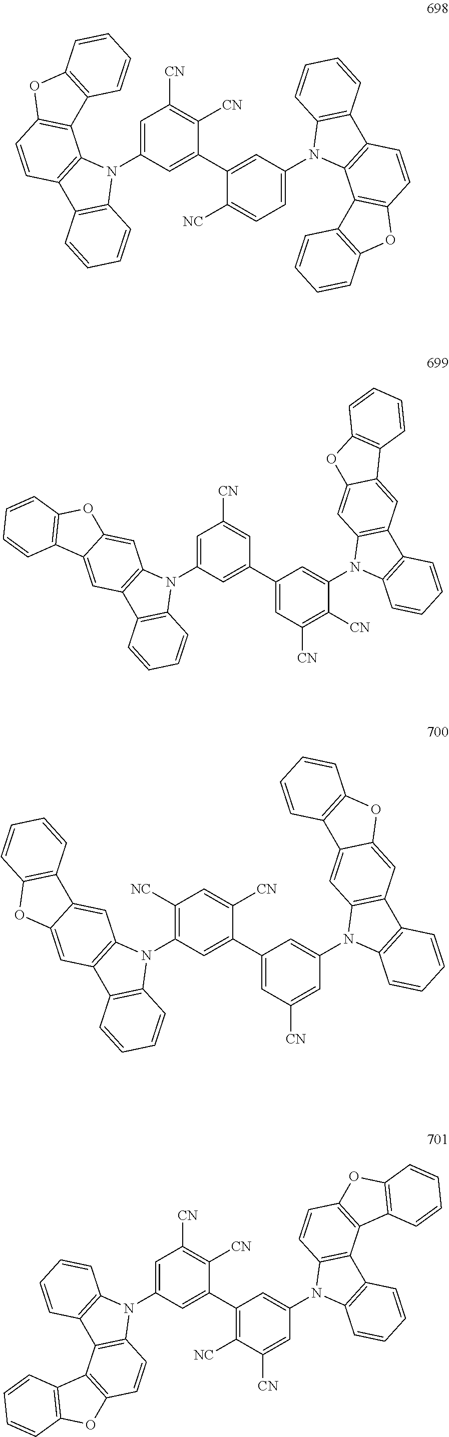 [SMILES: N#Cc1ccc(-n2c3ccccc3c3ccc4oc5ccccc5c4c32)cc1-c1cc(-n2c3ccccc3c3ccc4oc5ccccc5c4c32)cc(C#N)c1C#N ; N#Cc1cc(-c2cc(C#N)c(C#N)c(-n3c4ccccc4c4cc5c(cc43)oc3ccccc35)c2)cc(-n2c3ccccc3c3cc4c(cc32)oc2ccccc24)c1 ; N#Cc1cc(-c2cc(-n3c4ccccc4c4cc5oc6ccccc6c5cc43)c(C#N)cc2C#N)cc(-n2c3ccccc3c3cc4oc5ccccc5c4cc32)c1 ; N#Cc1cc(-n2c3ccccc3c3c4c(ccc32)oc2ccccc24)cc(-c2cc(-n3c4ccccc4c4c5c(ccc43)oc3ccccc35)cc(C#N)c2C#N)c1C#N]

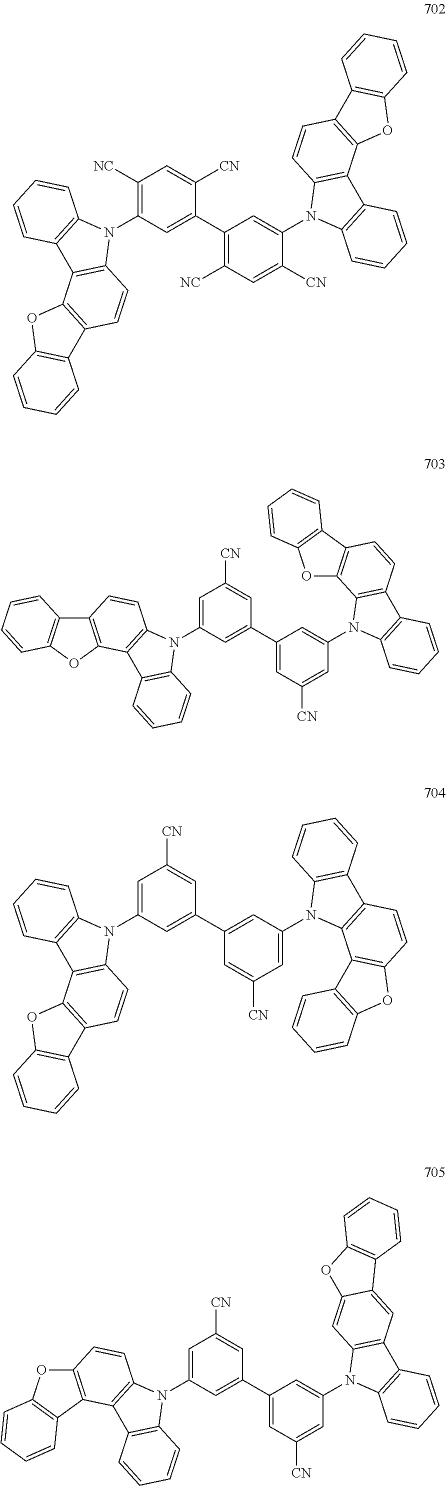 [SMILES: N#Cc1cc(C#N)c(-n2c3ccccc3c3c4oc5ccccc5c4ccc32)cc1-c1cc(-n2c3ccccc3c3c4oc5ccccc5c4ccc32)c(C#N)cc1C#N ; N#Cc1cc(-c2cc(C#N)cc(-n3c4ccccc4c4ccc5c6ccccc6oc5c43)c2)cc(-n2c3ccccc3c3c4oc5ccccc5c4ccc32)c1 ; N#Cc1cc(-c2cc(C#N)cc(-n3c4ccccc4c4ccc5oc6ccccc6c5c43)c2)cc(-n2c3ccccc3c3c4oc5ccccc5c4ccc32)c1 ; N#Cc1cc(-c2cc(C#N)cc(-n3c4ccccc4c4c5c(ccc43)oc3ccccc35)c2)cc(-n2c3ccccc3c3cc4c(cc32)oc2ccccc24)c1]

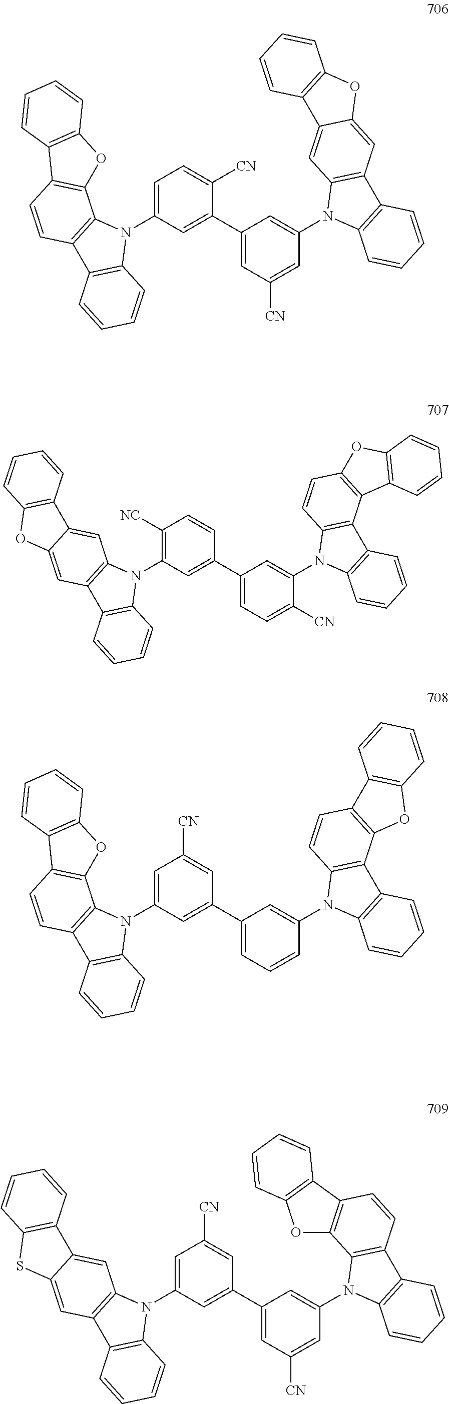 [SMILES: N#Cc1cc(-c2cc(-n3c4ccccc4c4ccc5c6ccccc6oc5c43)ccc2C#N)cc(-n2c3ccccc3c3cc4oc5ccccc5c4cc32)c1 ; N#Cc1ccc(-c2ccc(C#N)c(-n3c4ccccc4c4c5c(ccc43)oc3ccccc35)c2)cc1-n1c2ccccc2c2cc3oc4ccccc4c3cc21 ; N#Cc1cc(-c2cccc(-n3c4ccccc4c4c5oc6ccccc6c5ccc43)c2)cc(-n2c3ccccc3c3ccc4c5ccccc5oc4c32)c1 ; N#Cc1cc(-c2cc(C#N)cc(-n3c4ccccc4c4ccc5c6ccccc6oc5c43)c2)cc(-n2c3ccccc3c3cc4sc5ccccc5c4cc32)c1]

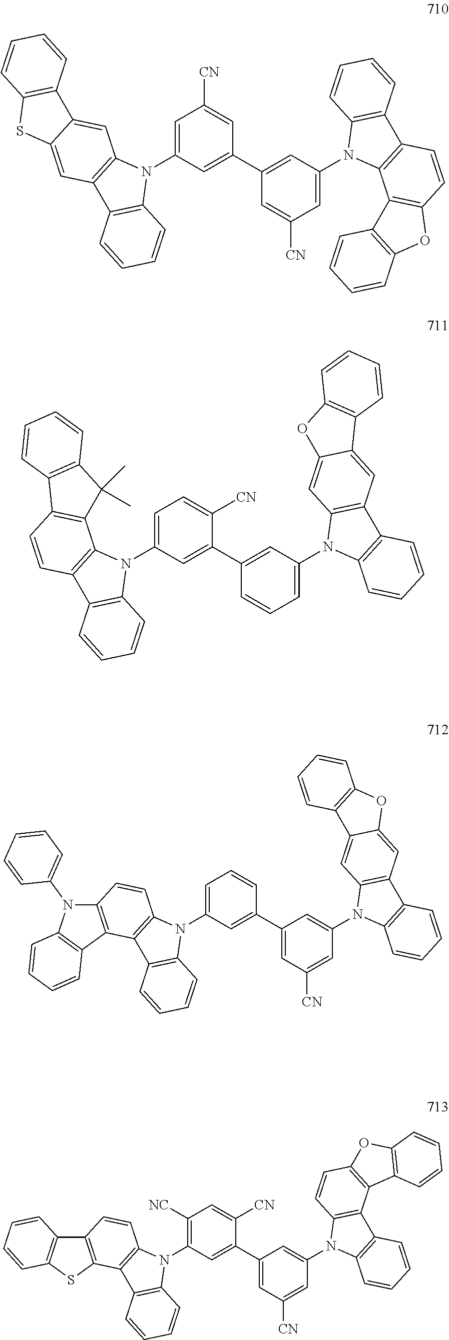 [SMILES: N#Cc1cc(-c2cc(C#N)cc(-n3c4ccccc4c4ccc5oc6ccccc6c5c43)c2)cc(-n2c3ccccc3c3cc4sc5ccccc5c4cc32)c1 ; CC1(C)c2ccccc2-c2ccc3c4ccccc4n(-c4ccc(C#N)c(-c5cccc(-n6c7ccccc7c7cc8c(cc76)oc6ccccc68)c5)c4)c3c21 ; N#Cc1cc(-c2cccc(-n3c4ccccc4c4c5c6ccccc6n(-c6ccccc6)c5ccc43)c2)cc(-n2c3ccccc3c3cc4oc5ccccc5c4cc32)c1 ; N#Cc1cc(-c2cc(-n3c4ccccc4c4c5sc6ccccc6c5ccc43)c(C#N)cc2C#N)cc(-n2c3ccccc3c3c4c(ccc32)oc2ccccc24)c1]

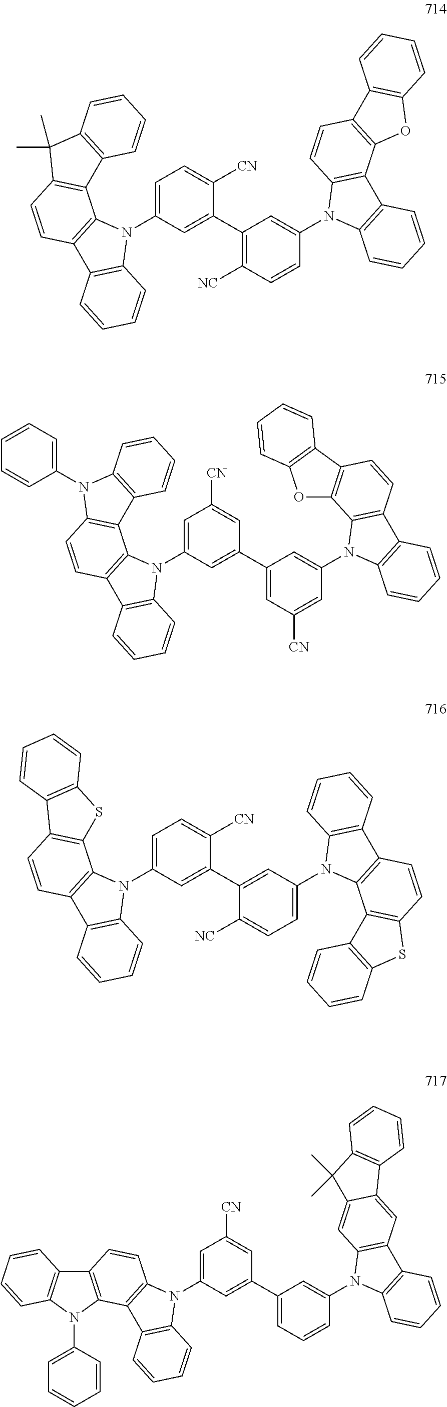 [SMILES: CC1(C)c2ccccc2-c2c1ccc1c3ccccc3n(-c3ccc(C#N)c(-c4cc(-n5c6ccccc6c6c7oc8ccccc8c7ccc65)ccc4C#N)c3)c21 ; N#Cc1cc(-c2cc(C#N)cc(-n3c4ccccc4c4ccc5c(c6ccccc6n5-c5ccccc5)c43)c2)cc(-n2c3ccccc3c3ccc4c5ccccc5oc4c32)c1 ; N#Cc1ccc(-n2c3ccccc3c3ccc4c5ccccc5sc4c32)cc1-c1cc(-n2c3ccccc3c3ccc4sc5ccccc5c4c32)ccc1C#N ; CC1(C)c2ccccc2-c2cc3c4ccccc4n(-c4cccc(-c5cc(C#N)cc(-n6c7ccccc7c7c6ccc6c8ccccc8n(-c8ccccc8)c67)c5)c4)c3cc21]

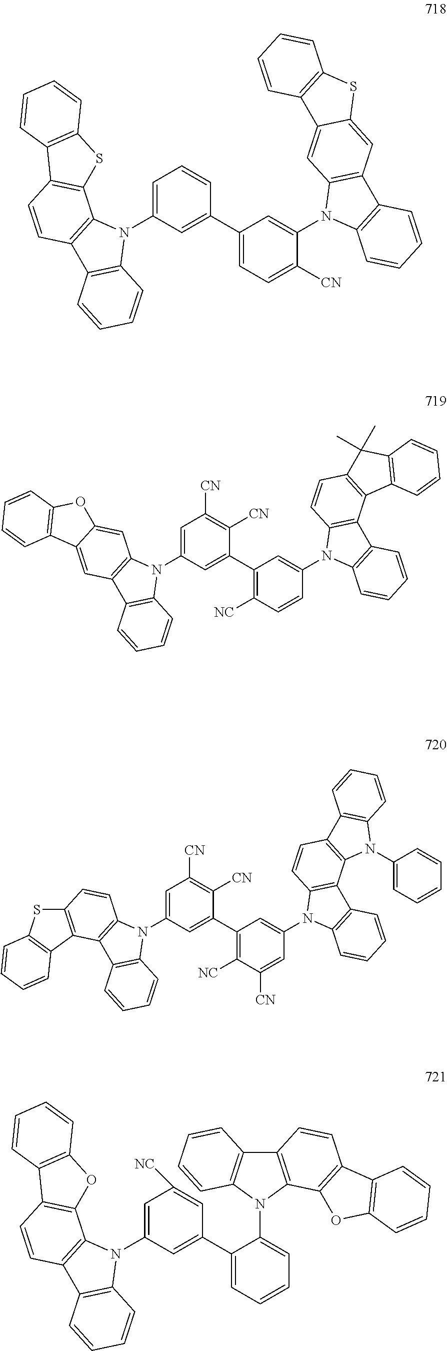 [SMILES: N#Cc1ccc(-c2cccc(-n3c4ccccc4c4ccc5c6ccccc6sc5c43)c2)cc1-n1c2ccccc2c2cc3sc4ccccc4c3cc21 ; CC1(C)c2ccccc2-c2c1ccc1c2c2ccccc2n1-c1ccc(C#N)c(-c2cc(-n3c4ccccc4c4cc5c(cc43)oc3ccccc35)cc(C#N)c2C#N)c1 ; N#Cc1cc(-n2c3ccccc3c3c4c(ccc32)sc2ccccc24)cc(-c2cc(-n3c4ccccc4c4c3ccc3c5ccccc5n(-c5ccccc5)c34)cc(C#N)c2C#N)c1C#N ; N#Cc1cc(-c2ccccc2-n2c3ccccc3c3ccc4c5ccccc5oc4c32)cc(-n2c3ccccc3c3ccc4c5ccccc5oc4c32)c1]

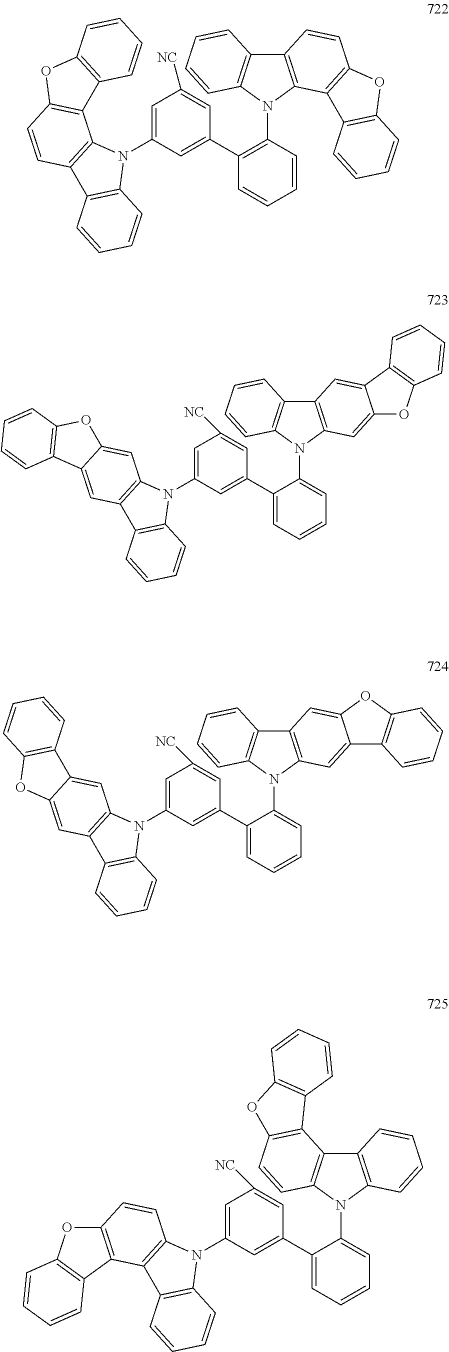 [SMILES: N#Cc1cc(-c2ccccc2-n2c3ccccc3c3ccc4oc5ccccc5c4c32)cc(-n2c3ccccc3c3ccc4oc5ccccc5c4c32)c1 ; N#Cc1cc(-c2ccccc2-n2c3ccccc3c3cc4c(cc32)oc2ccccc24)cc(-n2c3ccccc3c3cc4c(cc32)oc2ccccc24)c1 ; N#Cc1cc(-c2ccccc2-n2c3ccccc3c3cc4oc5ccccc5c4cc32)cc(-n2c3ccccc3c3cc4oc5ccccc5c4cc32)c1 ; N#Cc1cc(-c2ccccc2-n2c3ccccc3c3c4c(ccc32)oc2ccccc24)cc(-n2c3ccccc3c3c4c(ccc32)oc2ccccc24)c1]

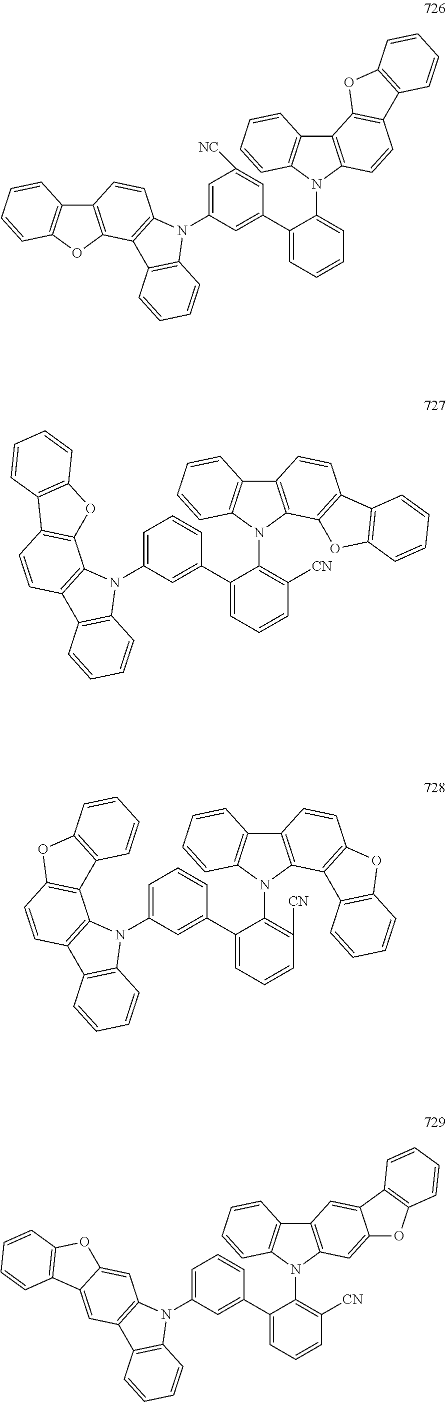 [SMILES: N#Cc1cc(-c2ccccc2-n2c3ccccc3c3c4oc5ccccc5c4ccc32)cc(-n2c3ccccc3c3c4oc5ccccc5c4ccc32)c1 ; N#Cc1cccc(-c2cccc(-n3c4ccccc4c4ccc5c6ccccc6oc5c43)c2)c1-n1c2ccccc2c2ccc3c4ccccc4oc3c21 ; N#Cc1cccc(-c2cccc(-n3c4ccccc4c4ccc5oc6ccccc6c5c43)c2)c1-n1c2ccccc2c2ccc3oc4ccccc4c3c21 ; N#Cc1cccc(-c2cccc(-n3c4ccccc4c4cc5c(cc43)oc3ccccc35)c2)c1-n1c2ccccc2c2cc3c(cc21)oc1ccccc13]

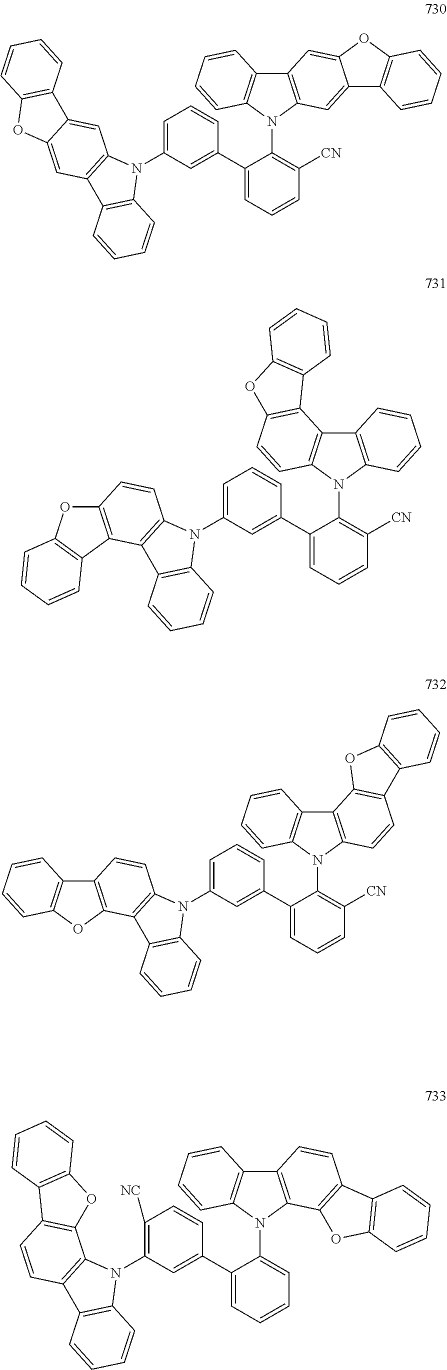 [SMILES: N#Cc1cccc(-c2cccc(-n3c4ccccc4c4cc5oc6ccccc6c5cc43)c2)c1-n1c2ccccc2c2cc3oc4ccccc4c3cc21 ; N#Cc1cccc(-c2cccc(-n3c4ccccc4c4c5c(ccc43)oc3ccccc35)c2)c1-n1c2ccccc2c2c3c(ccc21)oc1ccccc13 ; N#Cc1cccc(-c2cccc(-n3c4ccccc4c4c5oc6ccccc6c5ccc43)c2)c1-n1c2ccccc2c2c3oc4ccccc4c3ccc21 ; N#Cc1ccc(-c2ccccc2-n2c3ccccc3c3ccc4c5ccccc5oc4c32)cc1-n1c2ccccc2c2ccc3c4ccccc4oc3c21]

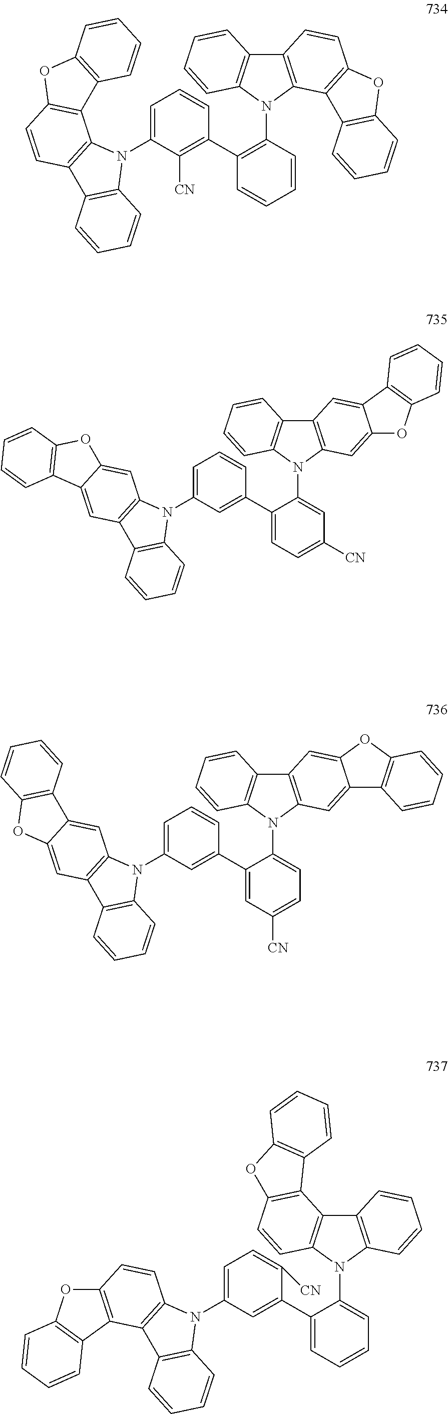 [SMILES: N#Cc1c(-c2ccccc2-n2c3ccccc3c3ccc4oc5ccccc5c4c32)cccc1-n1c2ccccc2c2ccc3oc4ccccc4c3c21 ; N#Cc1ccc(-c2cccc(-n3c4ccccc4c4cc5c(cc43)oc3ccccc35)c2)c(-n2c3ccccc3c3cc4c(cc32)oc2ccccc24)c1 ; N#Cc1ccc(-n2c3ccccc3c3cc4oc5ccccc5c4cc32)c(-c2cccc(-n3c4ccccc4c4cc5oc6ccccc6c5cc43)c2)c1 ; N#Cc1ccc(-n2c3ccccc3c3c4c(ccc32)oc2ccccc24)cc1-c1ccccc1-n1c2ccccc2c2c3c(ccc21)oc1ccccc13]

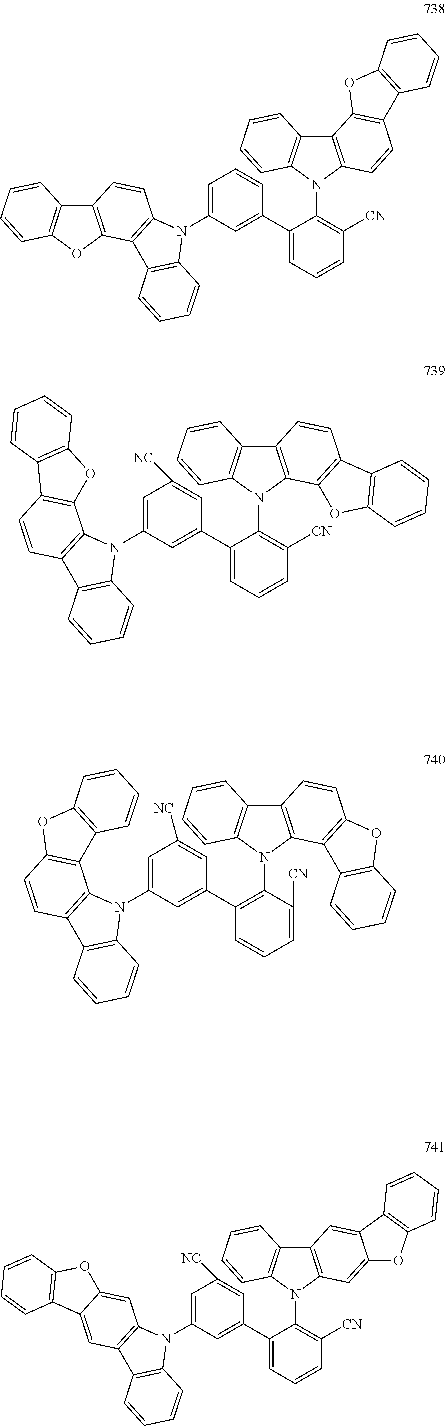 [SMILES: N#Cc1cccc(-c2cccc(-n3c4ccccc4c4c5oc6ccccc6c5ccc43)c2)c1-n1c2ccccc2c2c3oc4ccccc4c3ccc21 ; N#Cc1cc(-c2cccc(C#N)c2-n2c3ccccc3c3ccc4c5ccccc5oc4c32)cc(-n2c3ccccc3c3ccc4c5ccccc5oc4c32)c1 ; N#Cc1cc(-c2cccc(C#N)c2-n2c3ccccc3c3ccc4oc5ccccc5c4c32)cc(-n2c3ccccc3c3ccc4oc5ccccc5c4c32)c1 ; N#Cc1cc(-c2cccc(C#N)c2-n2c3ccccc3c3cc4c(cc32)oc2ccccc24)cc(-n2c3ccccc3c3cc4c(cc32)oc2ccccc24)c1]

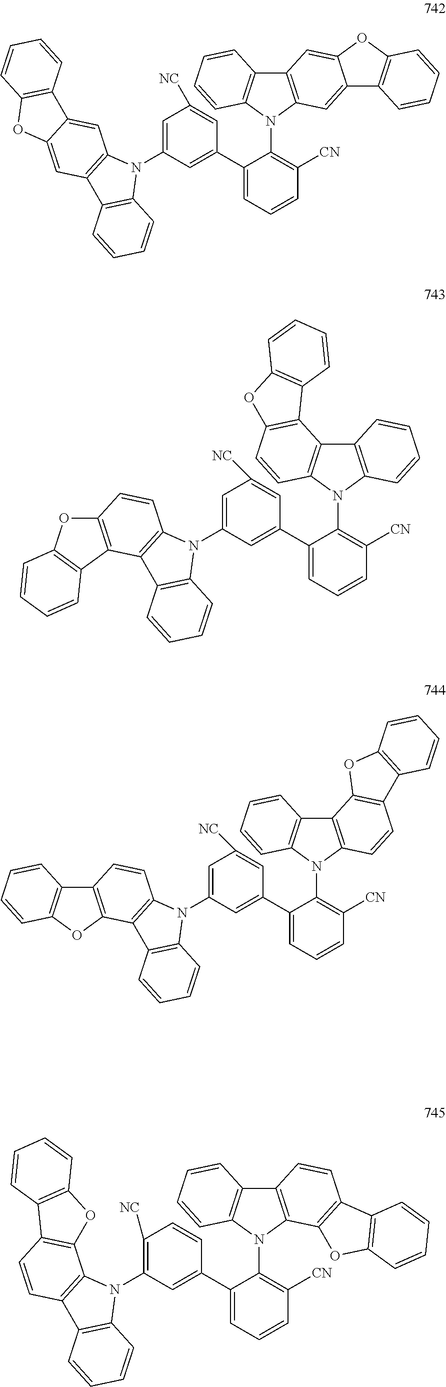 [SMILES: N#Cc1cc(-c2cccc(C#N)c2-n2c3ccccc3c3cc4oc5ccccc5c4cc32)cc(-n2c3ccccc3c3cc4oc5ccccc5c4cc32)c1 ; N#Cc1cc(-c2cccc(C#N)c2-n2c3ccccc3c3c4c(ccc32)oc2ccccc24)cc(-n2c3ccccc3c3c4c(ccc32)oc2ccccc24)c1 ; N#Cc1cc(-c2cccc(C#N)c2-n2c3ccccc3c3c4oc5ccccc5c4ccc32)cc(-n2c3ccccc3c3c4oc5ccccc5c4ccc32)c1 ; N#Cc1ccc(-c2cccc(C#N)c2-n2c3ccccc3c3ccc4c5ccccc5oc4c32)cc1-n1c2ccccc2c2ccc3c4ccccc4oc3c21]

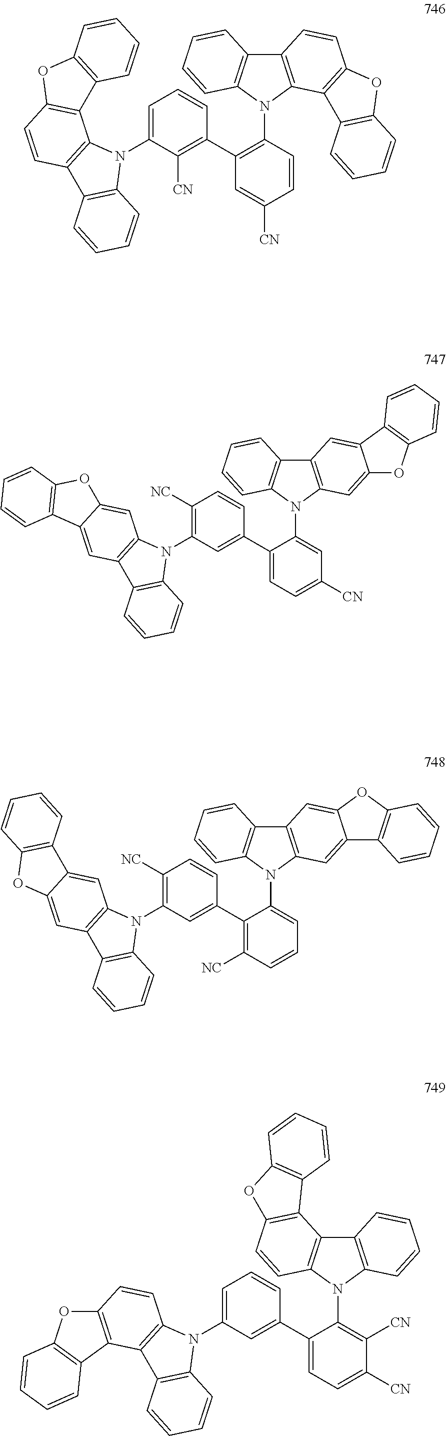 [SMILES: N#Cc1ccc(-n2c3ccccc3c3ccc4oc5ccccc5c4c32)c(-c2cccc(-n3c4ccccc4c4ccc5oc6ccccc6c5c43)c2C#N)c1 ; N#Cc1ccc(-c2ccc(C#N)c(-n3c4ccccc4c4cc5c(cc43)oc3ccccc35)c2)c(-n2c3ccccc3c3cc4c(cc32)oc2ccccc24)c1 ; N#Cc1ccc(-c2c(C#N)cccc2-n2c3ccccc3c3cc4oc5ccccc5c4cc32)cc1-n1c2ccccc2c2cc3oc4ccccc4c3cc21 ; N#Cc1ccc(-c2cccc(-n3c4ccccc4c4c5c(ccc43)oc3ccccc35)c2)c(-n2c3ccccc3c3c4c(ccc32)oc2ccccc24)c1C#N]

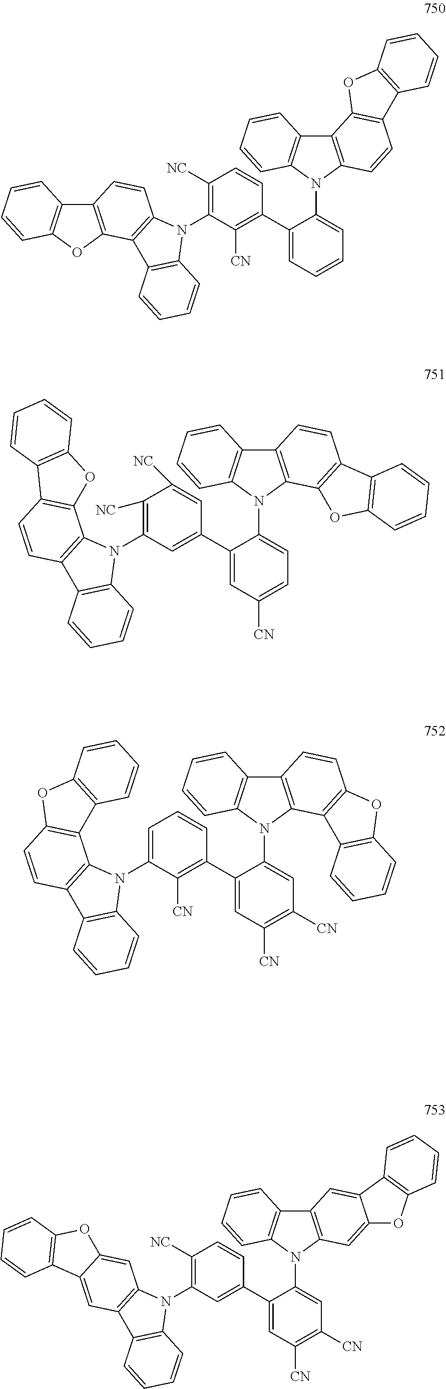 [SMILES: N#Cc1ccc(-c2ccccc2-n2c3ccccc3c3c4oc5ccccc5c4ccc32)c(C#N)c1-n1c2ccccc2c2c3oc4ccccc4c3ccc21 ; N#Cc1ccc(-n2c3ccccc3c3ccc4c5ccccc5oc4c32)c(-c2cc(C#N)c(C#N)c(-n3c4ccccc4c4ccc5c6ccccc6oc5c43)c2)c1 ; N#Cc1cc(-c2cccc(-n3c4ccccc4c4ccc5oc6ccccc6c5c43)c2C#N)c(-n2c3ccccc3c3ccc4oc5ccccc5c4c32)cc1C#N ; N#Cc1cc(-c2ccc(C#N)c(-n3c4ccccc4c4cc5c(cc43)oc3ccccc35)c2)c(-n2c3ccccc3c3cc4c(cc32)oc2ccccc24)cc1C#N]

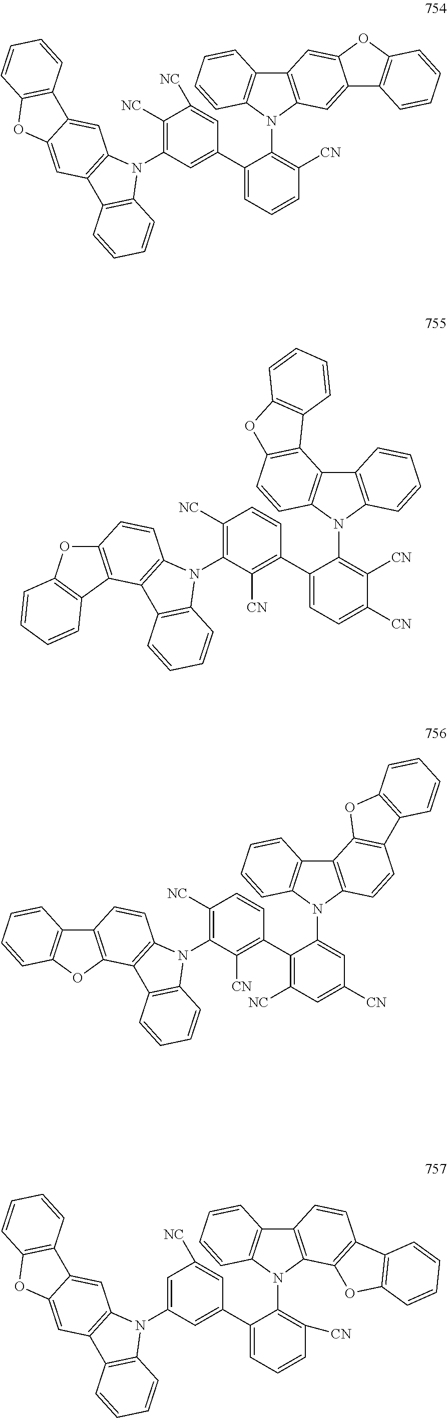 [SMILES: N#Cc1cc(-c2cccc(C#N)c2-n2c3ccccc3c3cc4oc5ccccc5c4cc32)cc(-n2c3ccccc3c3cc4oc5ccccc5c4cc32)c1C#N ; N#Cc1ccc(-c2ccc(C#N)c(-n3c4ccccc4c4c5c(ccc43)oc3ccccc35)c2C#N)c(-n2c3ccccc3c3c4c(ccc32)oc2ccccc24)c1C#N ; N#Cc1cc(C#N)c(-c2ccc(C#N)c(-n3c4ccccc4c4c5oc6ccccc6c5ccc43)c2C#N)c(-n2c3ccccc3c3c4oc5ccccc5c4ccc32)c1 ; N#Cc1cc(-c2cccc(C#N)c2-n2c3ccccc3c3ccc4c5ccccc5oc4c32)cc(-n2c3ccccc3c3cc4oc5ccccc5c4cc32)c1]

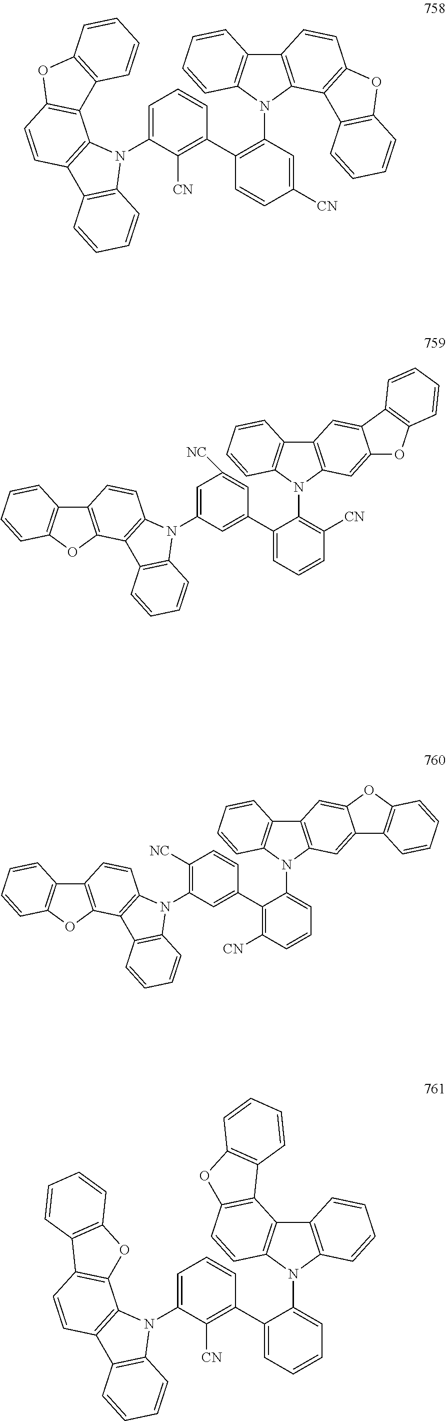

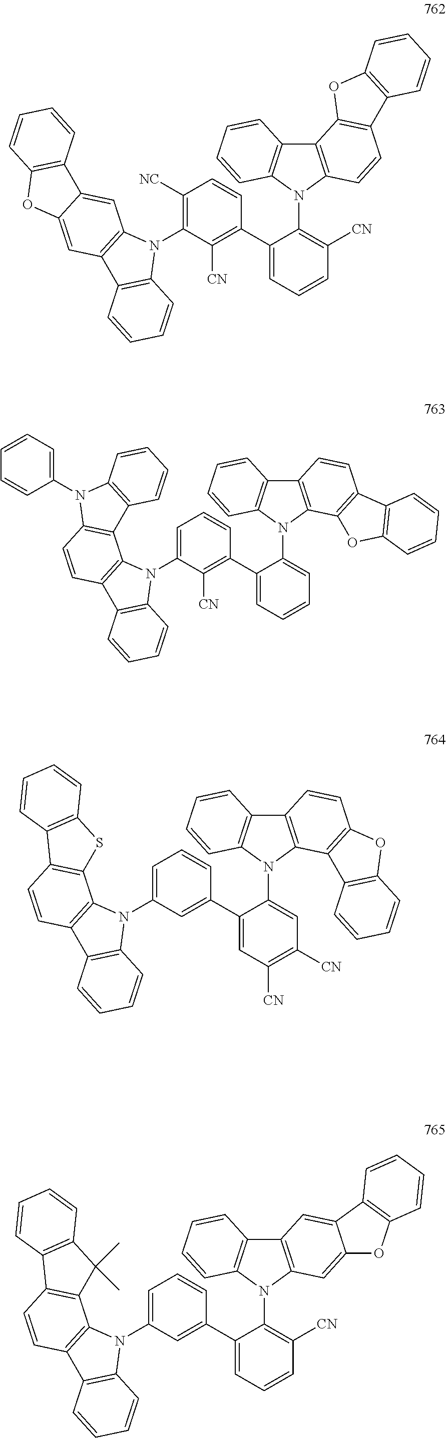 [SMILES: N#Cc1ccc(-c2cccc(C#N)c2-n2c3ccccc3c3c4oc5ccccc5c4ccc32)c(C#N)c1-n1c2ccccc2c2cc3oc4ccccc4c3cc21 ; N#Cc1c(-c2ccccc2-n2c3ccccc3c3ccc4c5ccccc5oc4c32)cccc1-n1c2ccccc2c2ccc3c(c4ccccc4n3-c3ccccc3)c21 ; N#Cc1cc(-c2cccc(-n3c4ccccc4c4ccc5c6ccccc6sc5c43)c2)c(-n2c3ccccc3c3ccc4oc5ccccc5c4c32)cc1C#N ; CC1(C)c2ccccc2-c2ccc3c4ccccc4n(-c4cccc(-c5cccc(C#N)c5-n5c6ccccc6c6cc7c(cc65)oc5ccccc57)c4)c3c21]

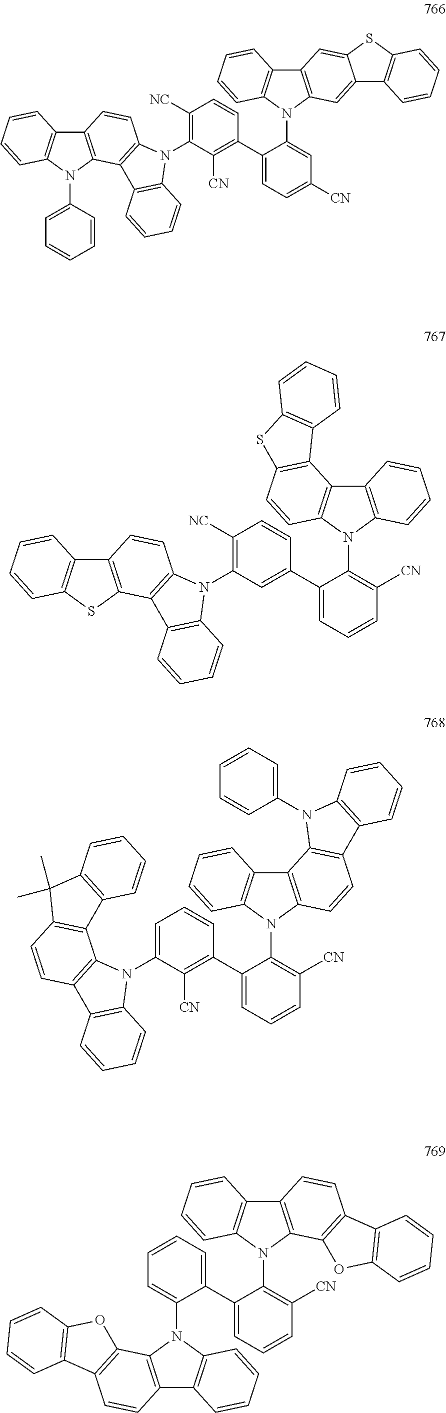 [SMILES: N#Cc1ccc(-c2ccc(C#N)c(-n3c4ccccc4c4c3ccc3c5ccccc5n(-c5ccccc5)c34)c2C#N)c(-n2c3ccccc3c3cc4sc5ccccc5c4cc32)c1 ; N#Cc1ccc(-c2cccc(C#N)c2-n2c3ccccc3c3c4c(ccc32)sc2ccccc24)cc1-n1c2ccccc2c2c3sc4ccccc4c3ccc21 ; CC1(C)c2ccccc2-c2c1ccc1c3ccccc3n(-c3cccc(-c4cccc(C#N)c4-n4c5ccccc5c5c4ccc4c6ccccc6n(-c6ccccc6)c45)c3C#N)c21 ; N#Cc1cccc(-c2ccccc2-n2c3ccccc3c3ccc4c5ccccc5oc4c32)c1-n1c2ccccc2c2ccc3c4ccccc4oc3c21]

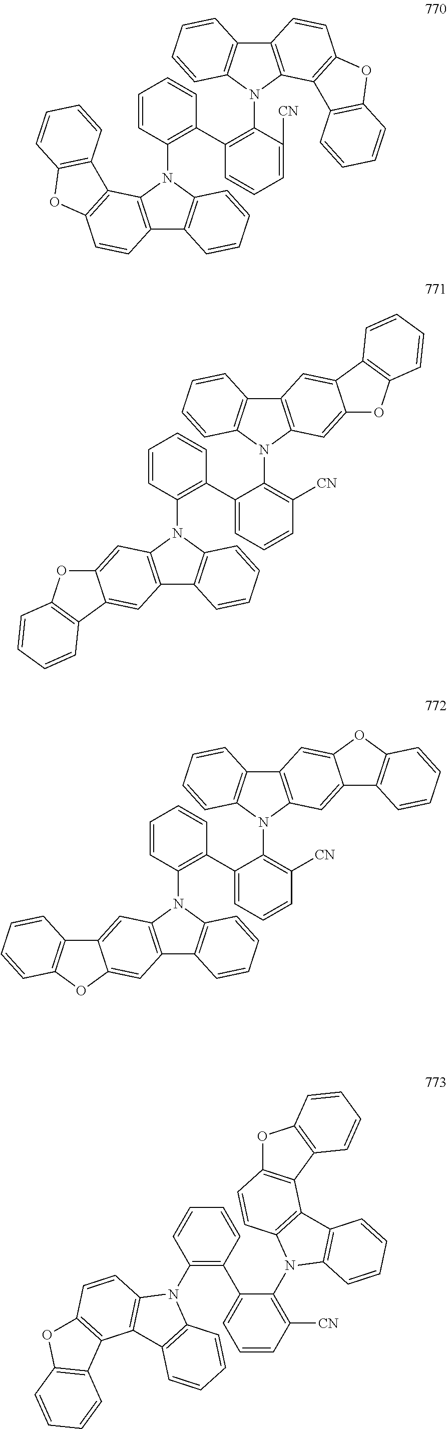 [SMILES: N#Cc1cccc(-c2ccccc2-n2c3ccccc3c3ccc4oc5ccccc5c4c32)c1-n1c2ccccc2c2ccc3oc4ccccc4c3c21 ; N#Cc1cccc(-c2ccccc2-n2c3ccccc3c3cc4c(cc32)oc2ccccc24)c1-n1c2ccccc2c2cc3c(cc21)oc1ccccc13 ; N#Cc1cccc(-c2ccccc2-n2c3ccccc3c3cc4oc5ccccc5c4cc32)c1-n1c2ccccc2c2cc3oc4ccccc4c3cc21 ; N#Cc1cccc(-c2ccccc2-n2c3ccccc3c3c4c(ccc32)oc2ccccc24)c1-n1c2ccccc2c2c3c(ccc21)oc1ccccc13]

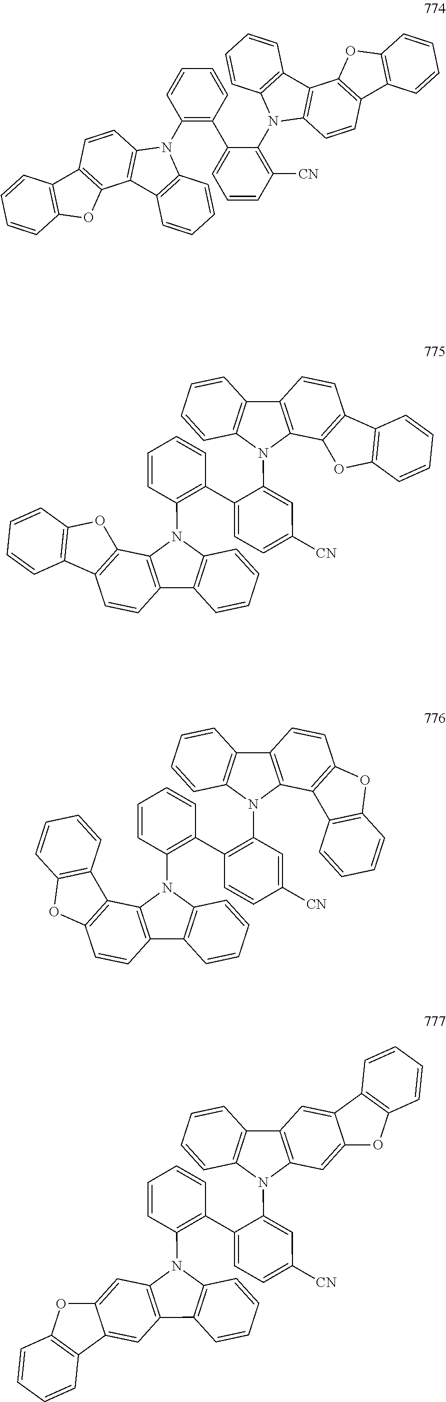 [SMILES: N#Cc1cccc(-c2ccccc2-n2c3ccccc3c3c4oc5ccccc5c4ccc32)c1-n1c2ccccc2c2c3oc4ccccc4c3ccc21 ; N#Cc1ccc(-c2ccccc2-n2c3ccccc3c3ccc4c5ccccc5oc4c32)c(-n2c3ccccc3c3ccc4c5ccccc5oc4c32)c1 ; N#Cc1ccc(-c2ccccc2-n2c3ccccc3c3ccc4oc5ccccc5c4c32)c(-n2c3ccccc3c3ccc4oc5ccccc5c4c32)c1 ; N#Cc1ccc(-c2ccccc2-n2c3ccccc3c3cc4c(cc32)oc2ccccc24)c(-n2c3ccccc3c3cc4c(cc32)oc2ccccc24)c1]

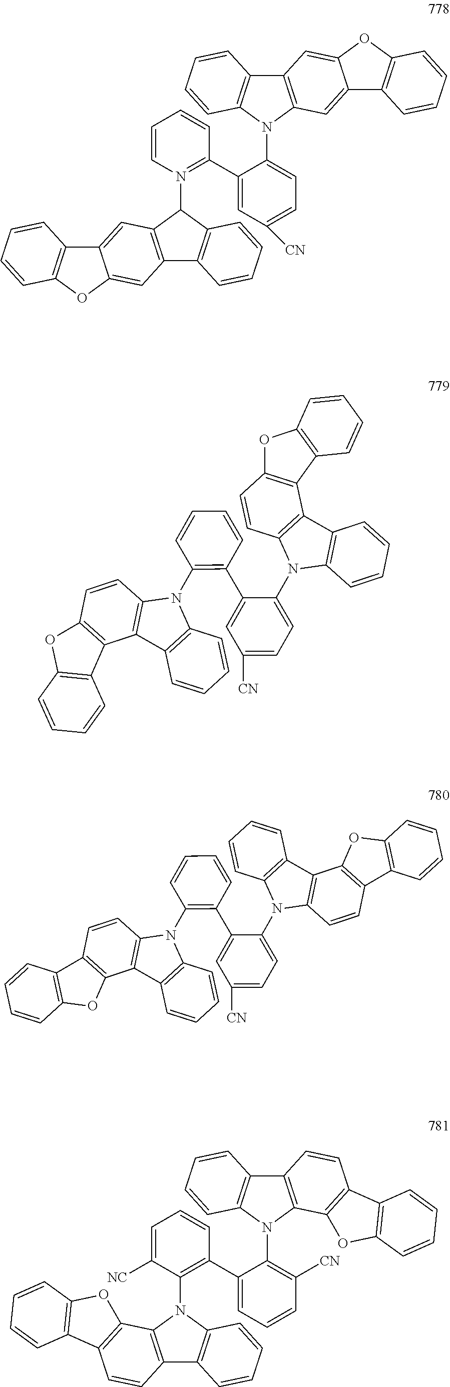

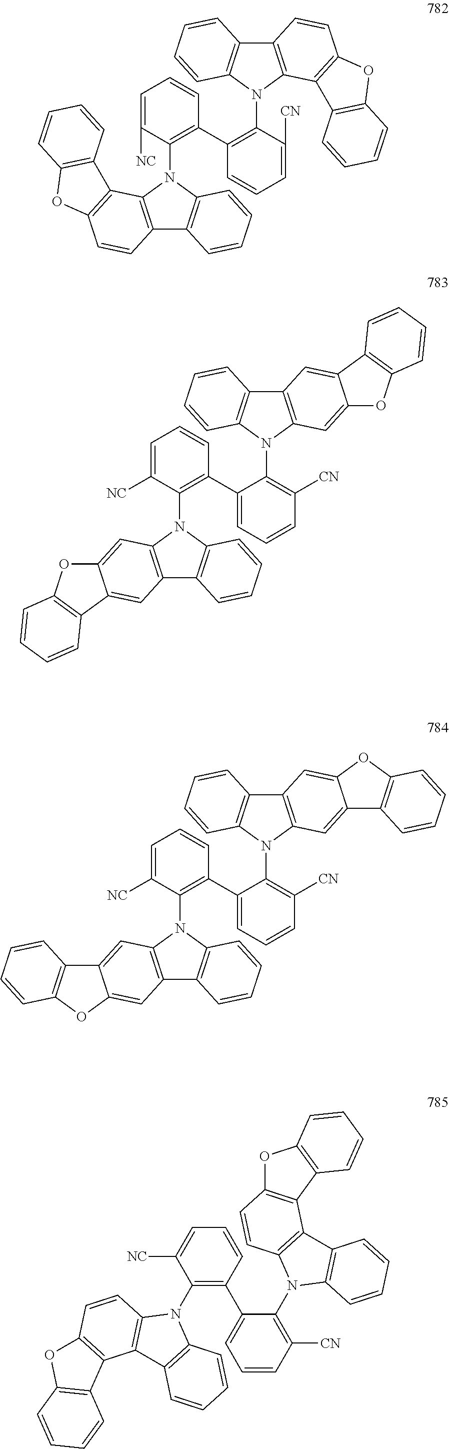

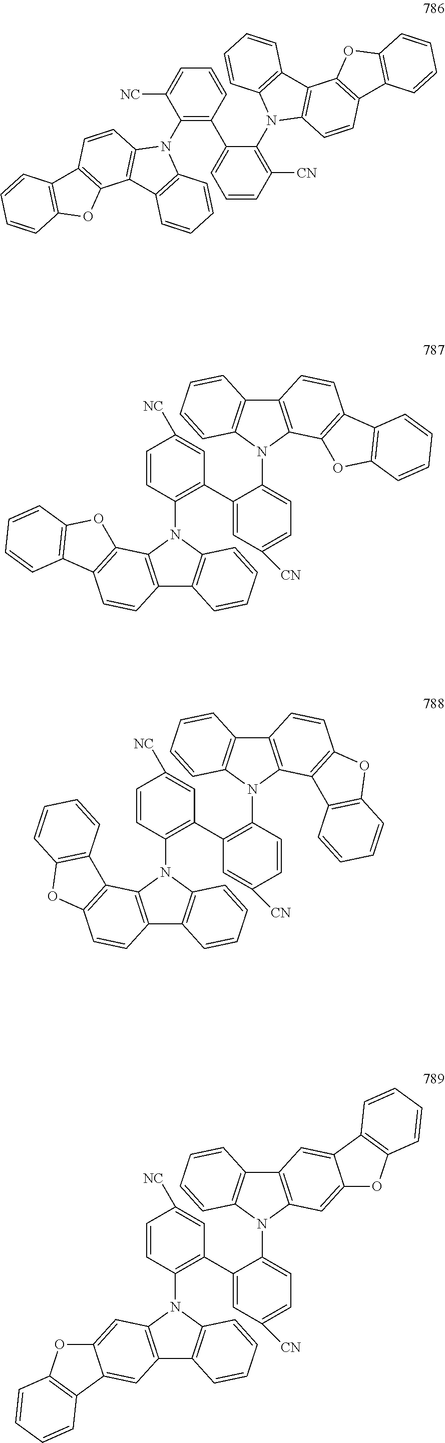 [SMILES: N#Cc1cccc(-c2cccc(C#N)c2-n2c3ccccc3c3c4oc5ccccc5c4ccc32)c1-n1c2ccccc2c2c3oc4ccccc4c3ccc21 ; N#Cc1ccc(-n2c3ccccc3c3ccc4c5ccccc5oc4c32)c(-c2cc(C#N)ccc2-n2c3ccccc3c3ccc4c5ccccc5oc4c32)c1 ; N#Cc1ccc(-n2c3ccccc3c3ccc4oc5ccccc5c4c32)c(-c2cc(C#N)ccc2-n2c3ccccc3c3ccc4oc5ccccc5c4c32)c1 ; N#Cc1ccc(-n2c3ccccc3c3cc4c(cc32)oc2ccccc24)c(-c2cc(C#N)ccc2-n2c3ccccc3c3cc4c(cc32)oc2ccccc24)c1]

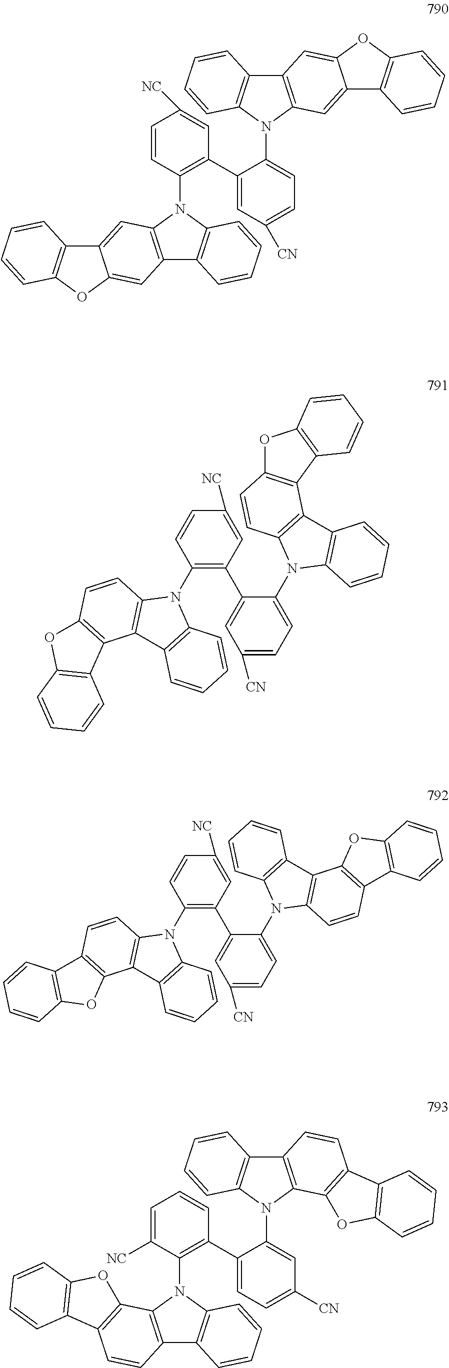 [SMILES: N#Cc1ccc(-n2c3ccccc3c3cc4oc5ccccc5c4cc32)c(-c2cc(C#N)ccc2-n2c3ccccc3c3cc4oc5ccccc5c4cc32)c1 ; N#Cc1ccc(-n2c3ccccc3c3c4c(ccc32)oc2ccccc24)c(-c2cc(C#N)ccc2-n2c3ccccc3c3c4c(ccc32)oc2ccccc24)c1 ; N#Cc1ccc(-n2c3ccccc3c3c4oc5ccccc5c4ccc32)c(-c2cc(C#N)ccc2-n2c3ccccc3c3c4oc5ccccc5c4ccc32)c1 ; N#Cc1ccc(-c2cccc(C#N)c2-n2c3ccccc3c3ccc4c5ccccc5oc4c32)c(-n2c3ccccc3c3ccc4c5ccccc5oc4c32)c1]

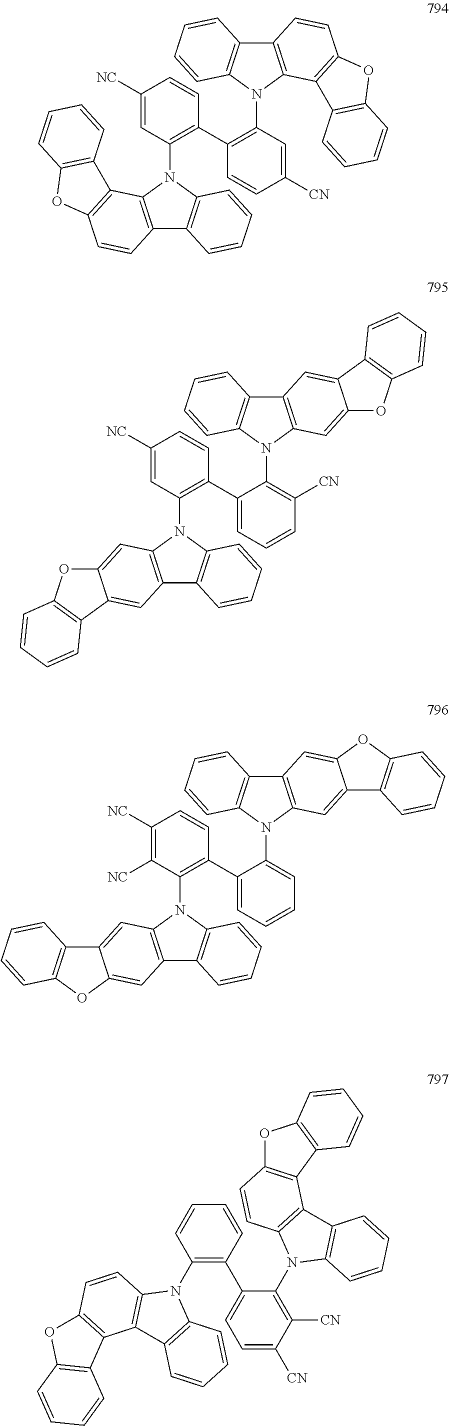 [SMILES: N#Cc1ccc(-c2ccc(C#N)cc2-n2c3ccccc3c3ccc4oc5ccccc5c4c32)c(-n2c3ccccc3c3ccc4oc5ccccc5c4c32)c1 ; N#Cc1ccc(-c2cccc(C#N)c2-n2c3ccccc3c3cc4c(cc32)oc2ccccc24)c(-n2c3ccccc3c3cc4c(cc32)oc2ccccc24)c1 ; N#Cc1ccc(-c2ccccc2-n2c3ccccc3c3cc4oc5ccccc5c4cc32)c(-n2c3ccccc3c3cc4oc5ccccc5c4cc32)c1C#N ; N#Cc1ccc(-c2ccccc2-n2c3ccccc3c3c4c(ccc32)oc2ccccc24)c(-n2c3ccccc3c3c4c(ccc32)oc2ccccc24)c1C#N]

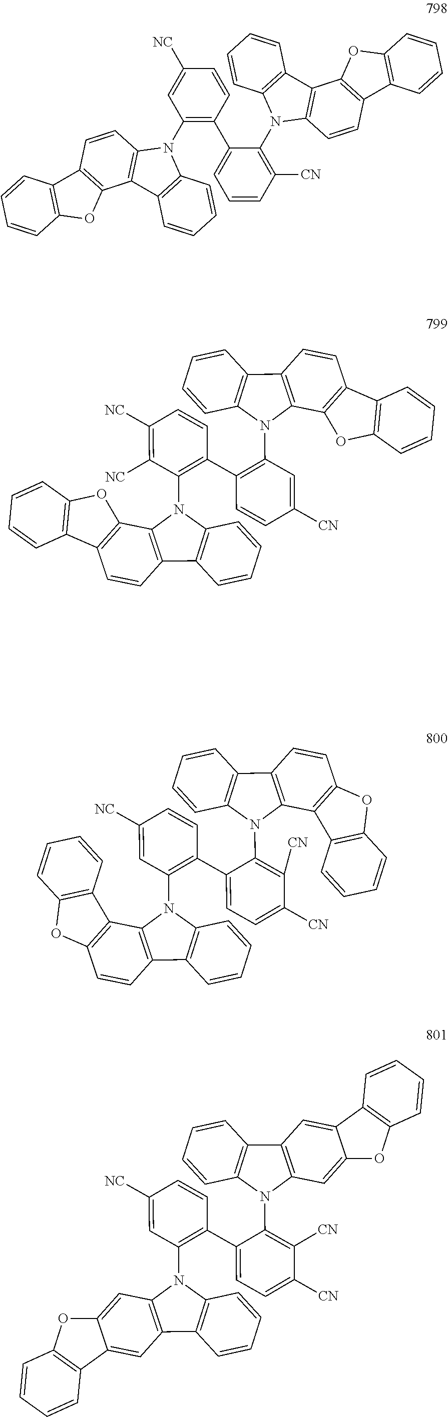 [SMILES: N#Cc1ccc(-c2cccc(C#N)c2-n2c3ccccc3c3c4oc5ccccc5c4ccc32)c(-n2c3ccccc3c3c4oc5ccccc5c4ccc32)c1 ; N#Cc1ccc(-c2ccc(C#N)c(C#N)c2-n2c3ccccc3c3ccc4c5ccccc5oc4c32)c(-n2c3ccccc3c3ccc4c5ccccc5oc4c32)c1 ; N#Cc1ccc(-c2ccc(C#N)c(C#N)c2-n2c3ccccc3c3ccc4oc5ccccc5c4c32)c(-n2c3ccccc3c3ccc4oc5ccccc5c4c32)c1 ; N#Cc1ccc(-c2ccc(C#N)c(C#N)c2-n2c3ccccc3c3cc4c(cc32)oc2ccccc24)c(-n2c3ccccc3c3cc4c(cc32)oc2ccccc24)c1]

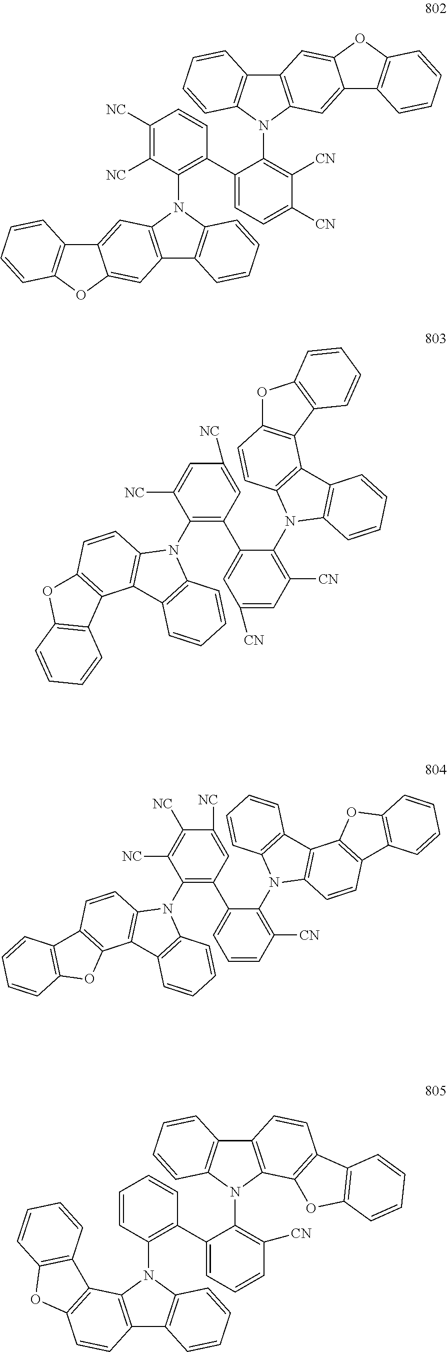 [SMILES: N#Cc1ccc(-c2ccc(C#N)c(C#N)c2-n2c3ccccc3c3cc4oc5ccccc5c4cc32)c(-n2c3ccccc3c3cc4oc5ccccc5c4cc32)c1C#N ; N#Cc1cc(C#N)c(-n2c3ccccc3c3c4c(ccc32)oc2ccccc24)c(-c2cc(C#N)cc(C#N)c2-n2c3ccccc3c3c4c(ccc32)oc2ccccc24)c1 ; N#Cc1cc(-c2cccc(C#N)c2-n2c3ccccc3c3c4oc5ccccc5c4ccc32)c(-n2c3ccccc3c3c4oc5ccccc5c4ccc32)c(C#N)c1C#N ; N#Cc1cccc(-c2ccccc2-n2c3ccccc3c3ccc4oc5ccccc5c4c32)c1-n1c2ccccc2c2ccc3c4ccccc4oc3c21]

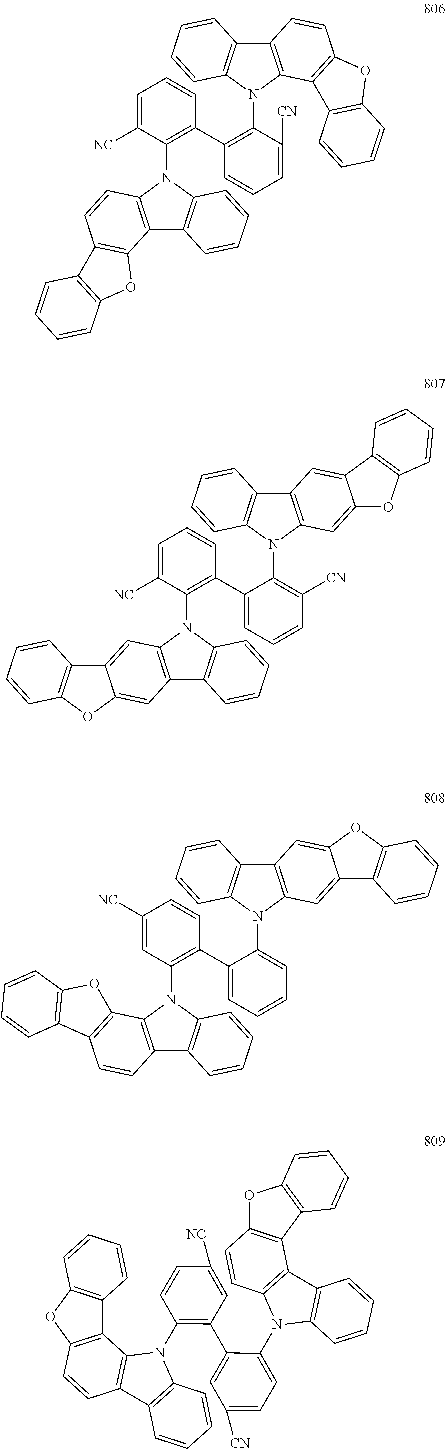 [SMILES: N#Cc1cccc(-c2cccc(C#N)c2-n2c3ccccc3c3ccc4oc5ccccc5c4c32)c1-n1c2ccccc2c2c3oc4ccccc4c3ccc21 ; N#Cc1cccc(-c2cccc(C#N)c2-n2c3ccccc3c3cc4oc5ccccc5c4cc32)c1-n1c2ccccc2c2cc3c(cc21)oc1ccccc13 ; N#Cc1ccc(-c2ccccc2-n2c3ccccc3c3cc4oc5ccccc5c4cc32)c(-n2c3ccccc3c3ccc4c5ccccc5oc4c32)c1 ; N#Cc1ccc(-n2c3ccccc3c3c4c(ccc32)oc2ccccc24)c(-c2cc(C#N)ccc2-n2c3ccccc3c3ccc4oc5ccccc5c4c32)c1]

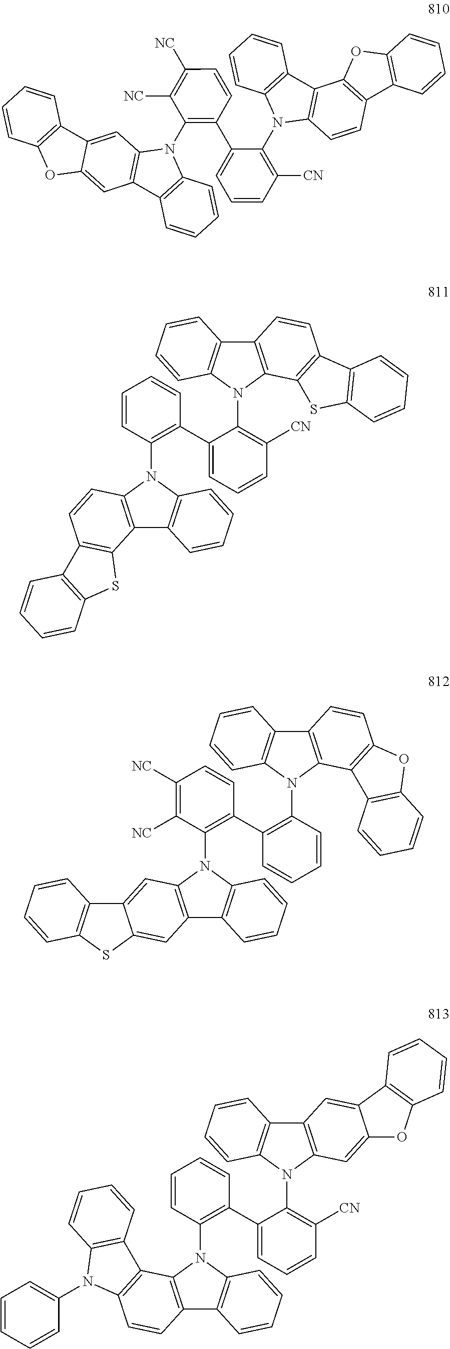 [SMILES: N#Cc1ccc(-c2cccc(C#N)c2-n2c3ccccc3c3c4oc5ccccc5c4ccc32)c(-n2c3ccccc3c3cc4oc5ccccc5c4cc32)c1C#N ; N#Cc1cccc(-c2ccccc2-n2c3ccccc3c3c4sc5ccccc5c4ccc32)c1-n1c2ccccc2c2ccc3c4ccccc4sc3c21 ; N#Cc1ccc(-c2ccccc2-n2c3ccccc3c3ccc4oc5ccccc5c4c32)c(-n2c3ccccc3c3cc4sc5ccccc5c4cc32)c1C#N ; N#Cc1cccc(-c2ccccc2-n2c3ccccc3c3ccc4c(c5ccccc5n4-c4ccccc4)c32)c1-n1c2ccccc2c2cc3c(cc21)oc1ccccc13]

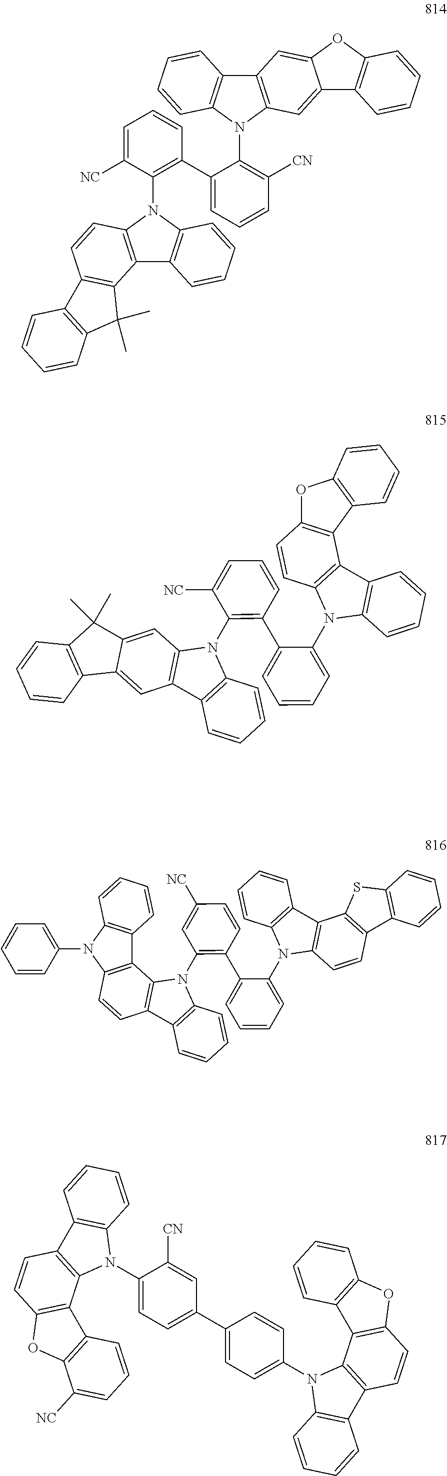 [SMILES: CC1(C)c2ccccc2-c2ccc3c(c21)c1ccccc1n3-c1c(C#N)cccc1-c1cccc(C#N)c1-n1c2ccccc2c2cc3oc4ccccc4c3cc21 ; CC1(C)c2ccccc2-c2cc3c4ccccc4n(-c4c(C#N)cccc4-c4ccccc4-n4c5ccccc5c5c6c(ccc54)oc4ccccc46)c3cc21 ; N#Cc1ccc(-c2ccccc2-n2c3ccccc3c3c4sc5ccccc5c4ccc32)c(-n2c3ccccc3c3ccc4c(c5ccccc5n4-c4ccccc4)c32)c1 ; N#Cc1cc(-c2ccc(-n3c4ccccc4c4ccc5oc6ccccc6c5c43)cc2)ccc1-n1c2ccccc2c2ccc3oc4c(C#N)cccc4c3c21]

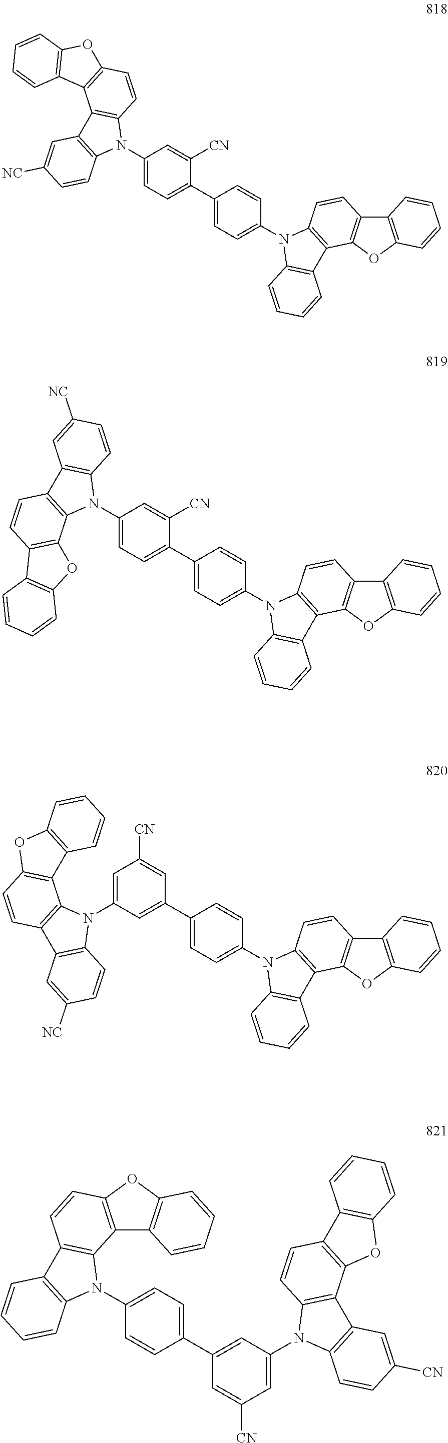 [SMILES: N#Cc1ccc2c(c1)c1c3c(ccc1n2-c1ccc(-c2ccc(-n4c5ccccc5c5c6oc7ccccc7c6ccc54)cc2)c(C#N)c1)oc1ccccc13 ; N#Cc1ccc2c(c1)c1ccc3c4ccccc4oc3c1n2-c1ccc(-c2ccc(-n3c4ccccc4c4c5oc6ccccc6c5ccc43)cc2)c(C#N)c1 ; N#Cc1cc(-c2ccc(-n3c4ccccc4c4c5oc6ccccc6c5ccc43)cc2)cc(-n2c3ccc(C#N)cc3c3ccc4oc5ccccc5c4c32)c1 ; N#Cc1cc(-c2ccc(-n3c4ccccc4c4ccc5oc6ccccc6c5c43)cc2)cc(-n2c3ccc(C#N)cc3c3c4oc5ccccc5c4ccc32)c1]

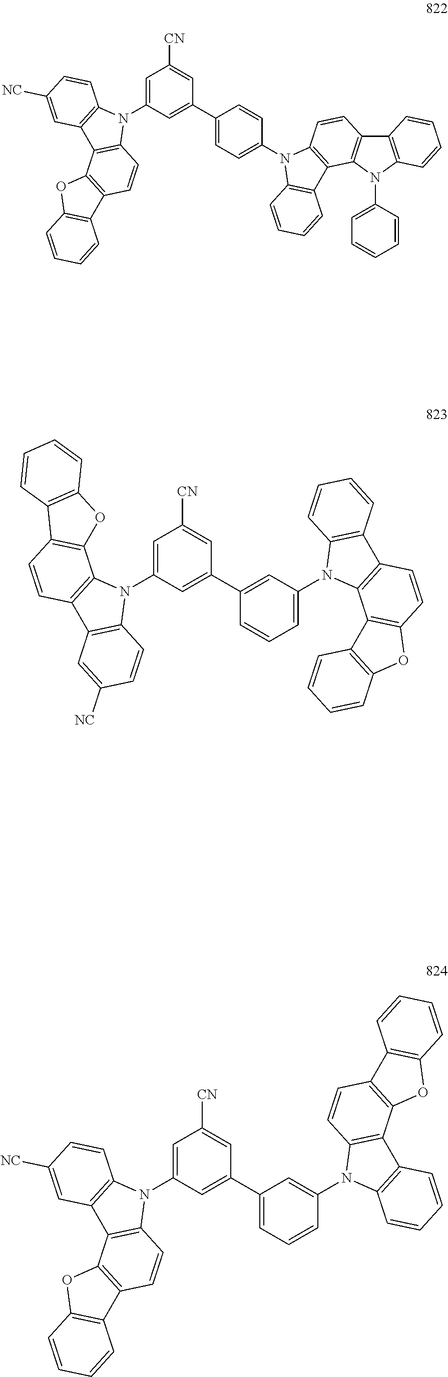 [SMILES: N#Cc1cc(-c2ccc(-n3c4ccccc4c4c3ccc3c5ccccc5n(-c5ccccc5)c34)cc2)cc(-n2c3ccc(C#N)cc3c3c4oc5ccccc5c4ccc32)c1 ; N#Cc1cc(-c2cccc(-n3c4ccccc4c4ccc5oc6ccccc6c5c43)c2)cc(-n2c3ccc(C#N)cc3c3ccc4c5ccccc5oc4c32)c1 ; N#Cc1cc(-c2cccc(-n3c4ccccc4c4c5oc6ccccc6c5ccc43)c2)cc(-n2c3ccc(C#N)cc3c3c4oc5ccccc5c4ccc32)c1]

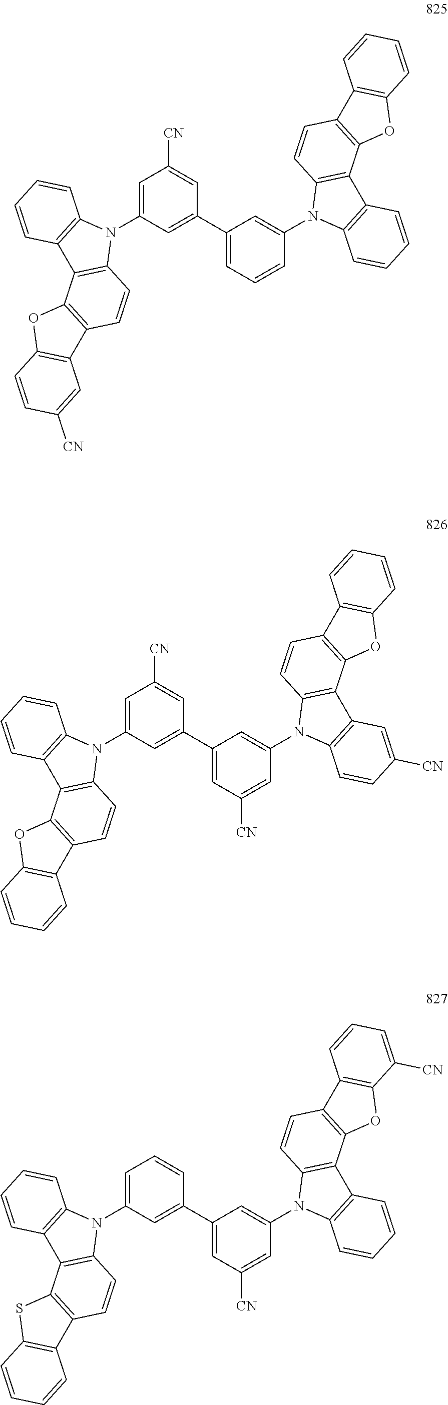 [SMILES: N#Cc1cc(-c2cccc(-n3c4ccccc4c4c5oc6ccccc6c5ccc43)c2)cc(-n2c3ccccc3c3c4oc5ccc(C#N)cc5c4ccc32)c1 ; N#Cc1cc(-c2cc(C#N)cc(-n3c4ccc(C#N)cc4c4c5oc6ccccc6c5ccc43)c2)cc(-n2c3ccccc3c3c4oc5ccccc5c4ccc32)c1 ; N#Cc1cc(-c2cccc(-n3c4ccccc4c4c5sc6ccccc6c5ccc43)c2)cc(-n2c3ccccc3c3c4oc5c(C#N)cccc5c4ccc32)c1]

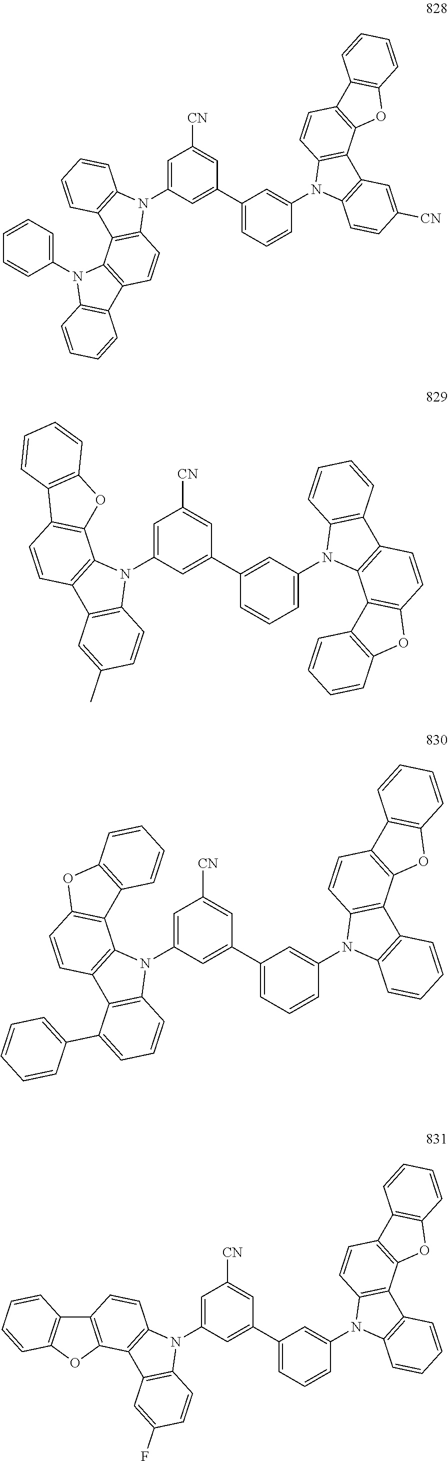 [SMILES: N#Cc1cc(-c2cccc(-n3c4ccc(C#N)cc4c4c5oc6ccccc6c5ccc43)c2)cc(-n2c3ccccc3c3c2ccc2c4ccccc4n(-c4ccccc4)c23)c1 ; Cc1ccc2c(c1)c1ccc3c4ccccc4oc3c1n2-c1cc(C#N)cc(-c2cccc(-n3c4ccccc4c4ccc5oc6ccccc6c5c43)c2)c1 ; N#Cc1cc(-c2cccc(-n3c4ccccc4c4c5oc6ccccc6c5ccc43)c2)cc(-n2c3cccc(-c4ccccc4)c3c3ccc4oc5ccccc5c4c32)c1 ; N#Cc1cc(-c2cccc(-n3c4ccccc4c4c5oc6ccccc6c5ccc43)c2)cc(-n2c3ccc(F)cc3c3c4oc5ccccc5c4ccc32)c1]

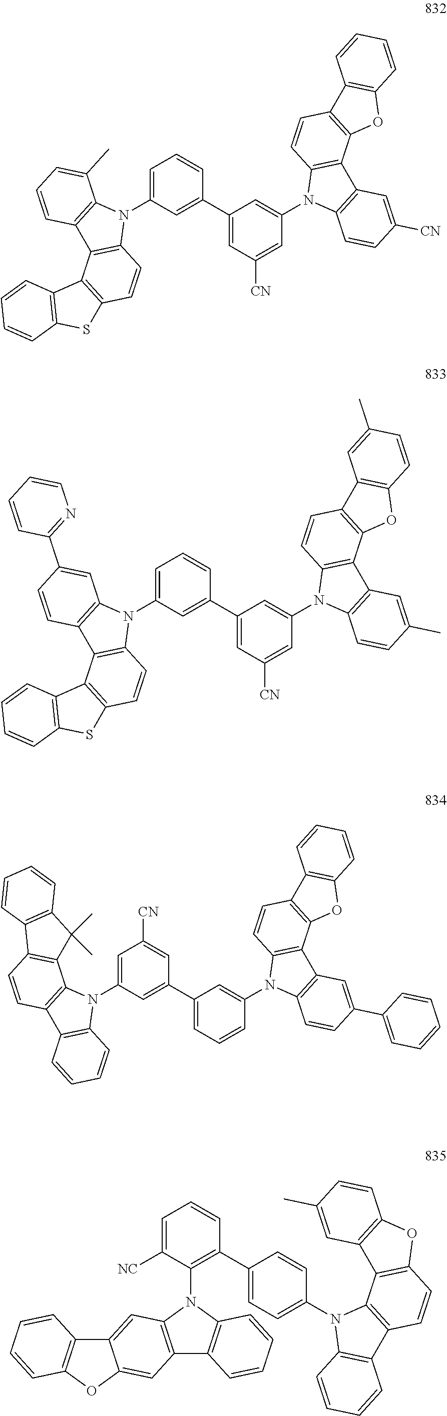 [SMILES: Cc1cccc2c3c4c(ccc3n(-c3cccc(-c5cc(C#N)cc(-n6c7ccc(C#N)cc7c7c8oc9ccccc9c8ccc76)c5)c3)c12)sc1ccccc14 ; Cc1ccc2oc3c(ccc4c3c3cc(C)ccc3n4-c3cc(C#N)cc(-c4cccc(-n5c6cc(-c7ccccn7)ccc6c6c7c(ccc65)sc5ccccc57)c4)c3)c2c1 ; CC1(C)c2ccccc2-c2ccc3c4ccccc4n(-c4cc(C#N)cc(-c5cccc(-n6c7ccc(-c8ccccc8)cc7c7c8oc9ccccc9c8ccc76)c5)c4)c3c21 ; Cc1ccc2oc3ccc4c5ccccc5n(-c5ccc(-c6cccc(C#N)c6-n6c7ccccc7c7cc8oc9ccccc9c8cc76)cc5)c4c3c2c1]

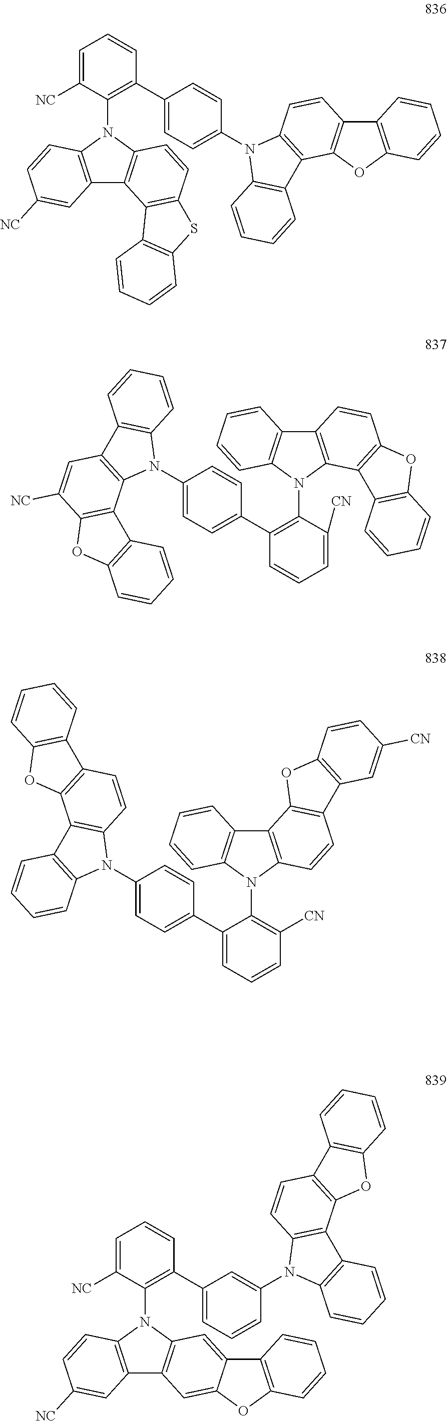 [SMILES: N#Cc1ccc2c(c1)c1c3c(ccc1n2-c1c(C#N)cccc1-c1ccc(-n2c4ccccc4c4c5oc6ccccc6c5ccc42)cc1)sc1ccccc13 ; N#Cc1cccc(-c2ccc(-n3c4ccccc4c4cc(C#N)c5oc6ccccc6c5c43)cc2)c1-n1c2ccccc2c2ccc3oc4ccccc4c3c21 ; N#Cc1ccc2oc3c(ccc4c3c3ccccc3n4-c3c(C#N)cccc3-c3ccc(-n4c5ccccc5c5c6oc7ccccc7c6ccc54)cc3)c2c1 ; N#Cc1ccc2c(c1)c1cc3oc4ccccc4c3cc1n2-c1c(C#N)cccc1-c1cccc(-n2c3ccccc3c3c4oc5ccccc5c4ccc32)c1]

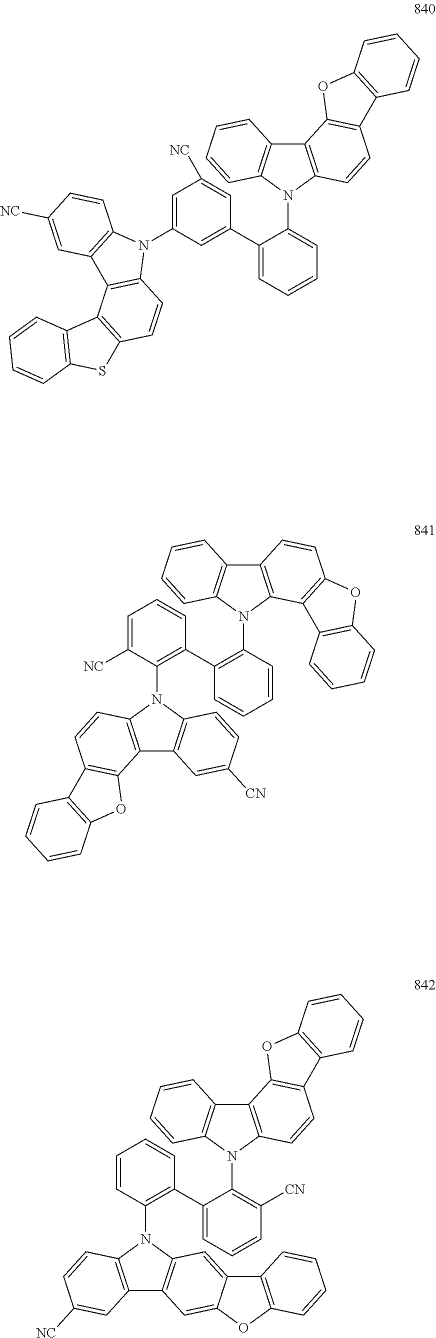 [SMILES: N#Cc1cc(-c2ccccc2-n2c3ccccc3c3c4oc5ccccc5c4ccc32)cc(-n2c3ccc(C#N)cc3c3c4c(ccc32)sc2ccccc24)c1 ; N#Cc1ccc2c(c1)c1c3oc4ccccc4c3ccc1n2-c1c(C#N)cccc1-c1ccccc1-n1c2ccccc2c2ccc3oc4ccccc4c3c21 ; N#Cc1ccc2c(c1)c1cc3oc4ccccc4c3cc1n2-c1ccccc1-c1cccc(C#N)c1-n1c2ccccc2c2c3oc4ccccc4c3ccc21]

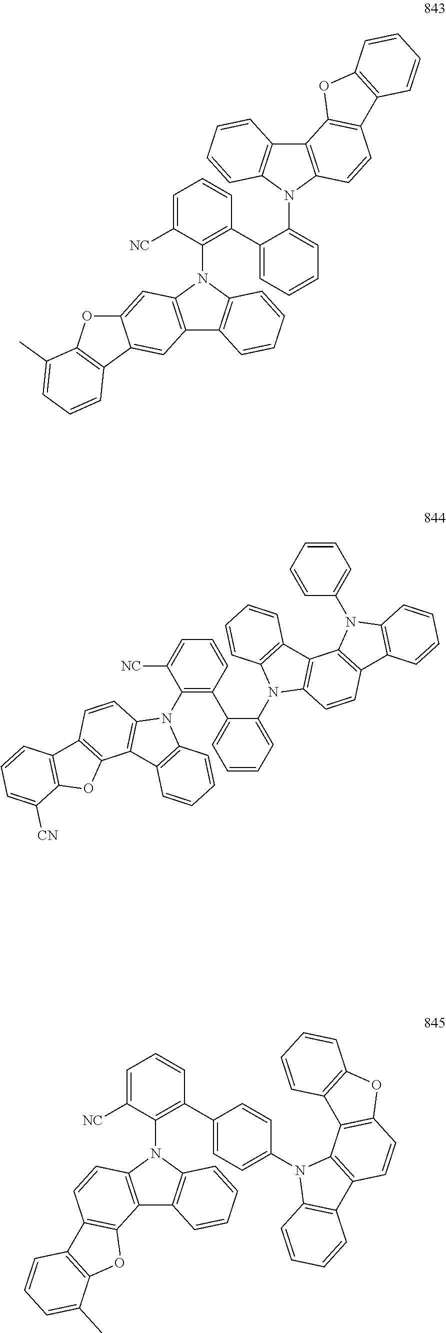 [SMILES: Cc1cccc2c1oc1cc3c(cc12)c1ccccc1n3-c1c(C#N)cccc1-c1ccccc1-n1c2ccccc2c2c3oc4ccccc4c3ccc21 ; N#Cc1cccc(-c2ccccc2-n2c3ccccc3c3c2ccc2c4ccccc4n(-c4ccccc4)c23)c1-n1c2ccccc2c2c3oc4c(C#N)cccc4c3ccc21 ; Cc1cccc2c1oc1c2ccc2c1c1ccccc1n2-c1c(C#N)cccc1-c1ccc(-n2c3ccccc3c3ccc4oc5ccccc5c4c32)cc1]

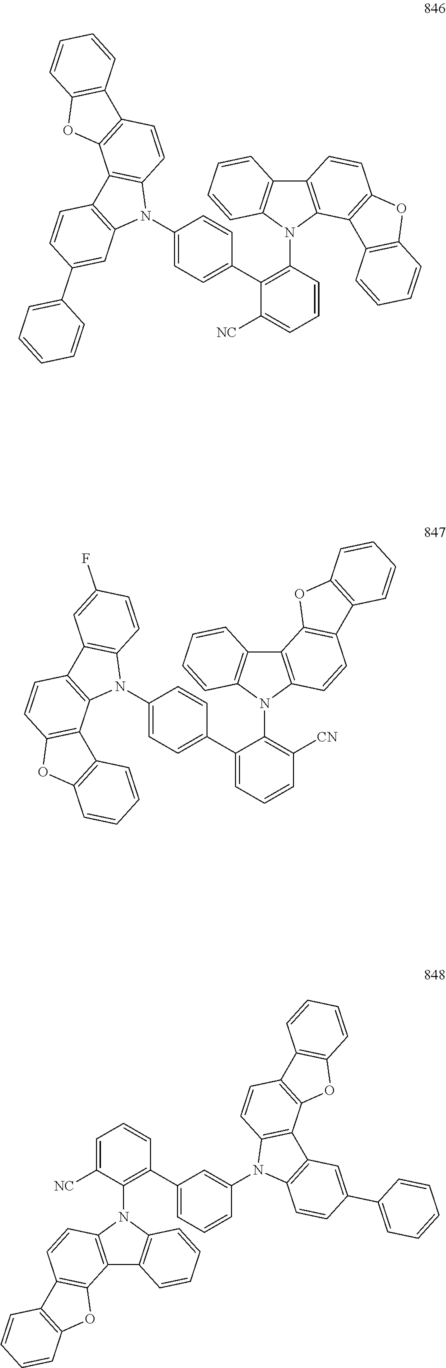 [SMILES: N#Cc1cccc(-n2c3ccccc3c3ccc4oc5ccccc5c4c32)c1-c1ccc(-n2c3cc(-c4ccccc4)ccc3c3c4oc5ccccc5c4ccc32)cc1 ; N#Cc1cccc(-c2ccc(-n3c4ccc(F)cc4c4ccc5oc6ccccc6c5c43)cc2)c1-n1c2ccccc2c2c3oc4ccccc4c3ccc21 ; N#Cc1cccc(-c2cccc(-n3c4ccc(-c5ccccc5)cc4c4c5oc6ccccc6c5ccc43)c2)c1-n1c2ccccc2c2c3oc4ccccc4c3ccc21]

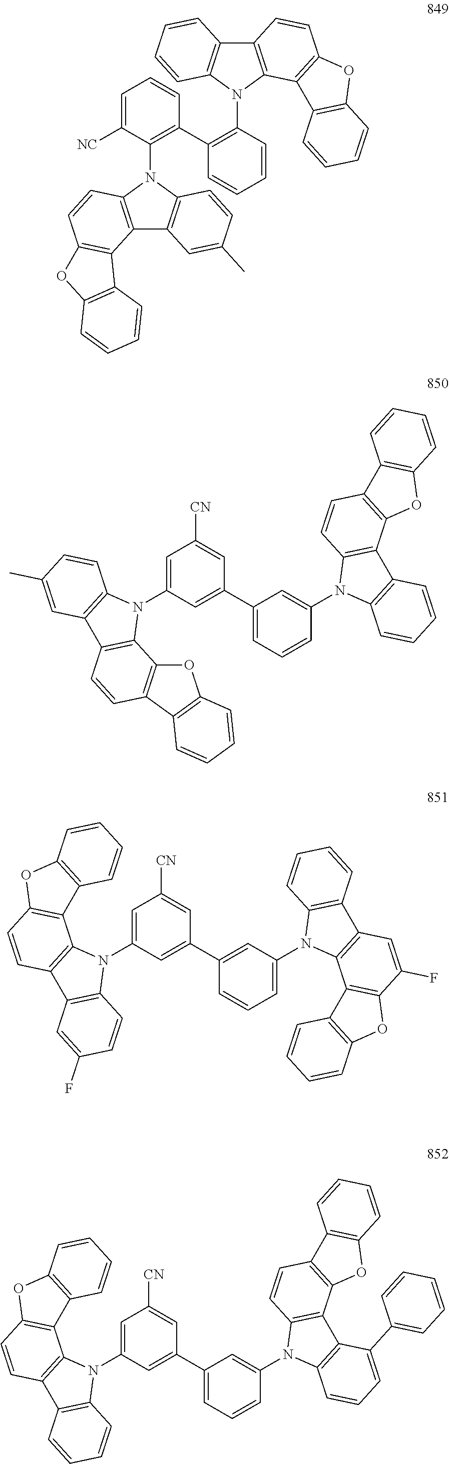 [SMILES: Cc1ccc2c(c1)c1c3c(ccc1n2-c1c(C#N)cccc1-c1ccccc1-n1c2ccccc2c2ccc4oc5ccccc5c4c21)oc1ccccc13 ; Cc1ccc2c(c1)c1ccc3c4ccccc4oc3c1n2-c1cc(C#N)cc(-c2cccc(-n3c4ccccc4c4c5oc6ccccc6c5ccc43)c2)c1 ; N#Cc1cc(-c2cccc(-n3c4ccccc4c4cc(F)c5oc6ccccc6c5c43)c2)cc(-n2c3ccc(F)cc3c3ccc4oc5ccccc5c4c32)c1 ; N#Cc1cc(-c2cccc(-n3c4cccc(-c5ccccc5)c4c4c5oc6ccccc6c5ccc43)c2)cc(-n2c3ccccc3c3ccc4oc5ccccc5c4c32)c1]

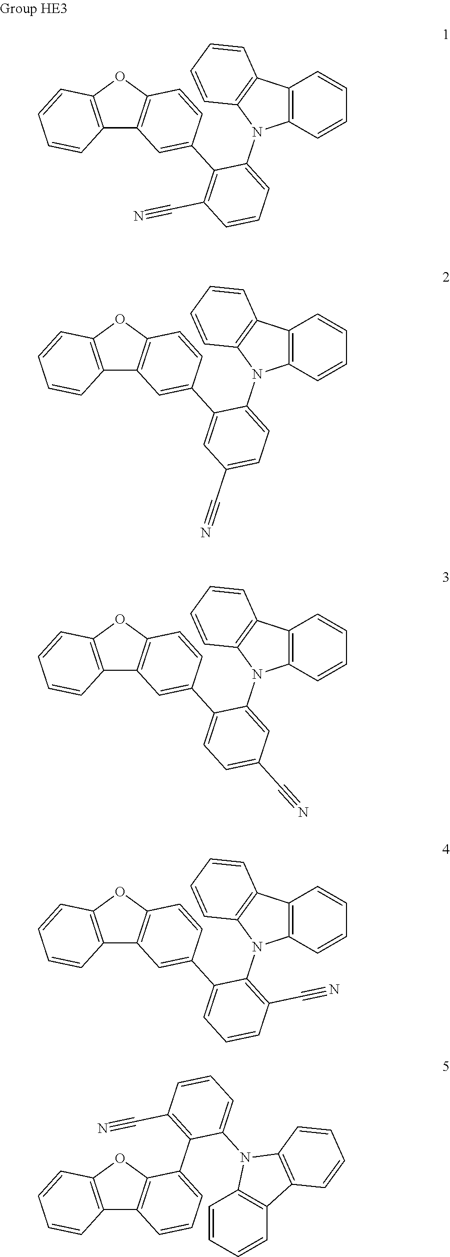

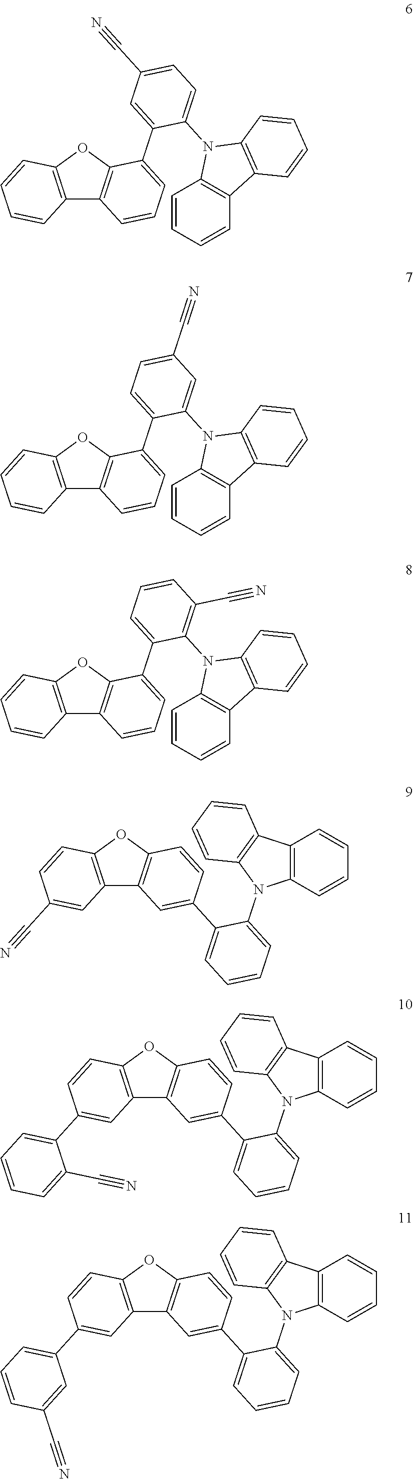

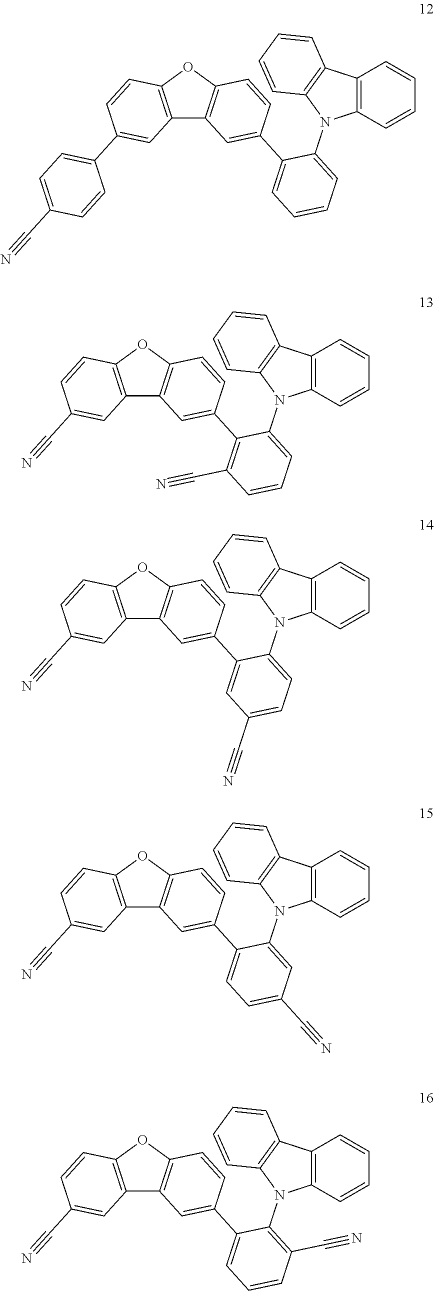

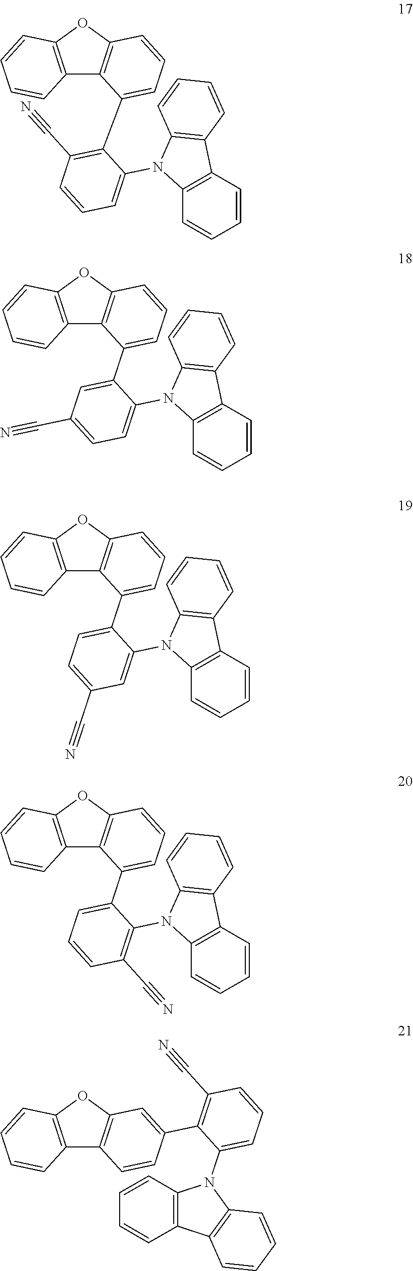

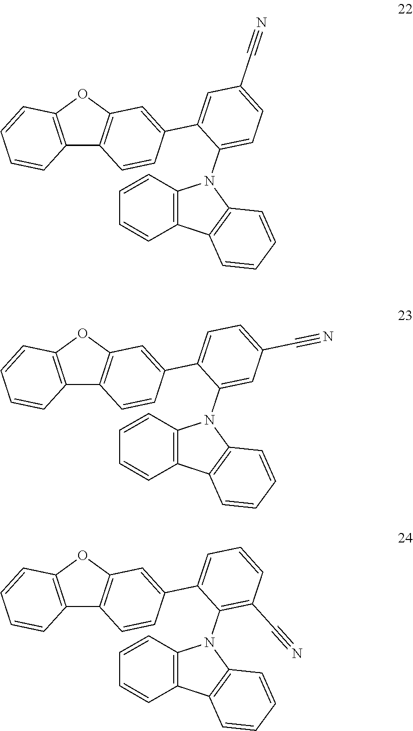

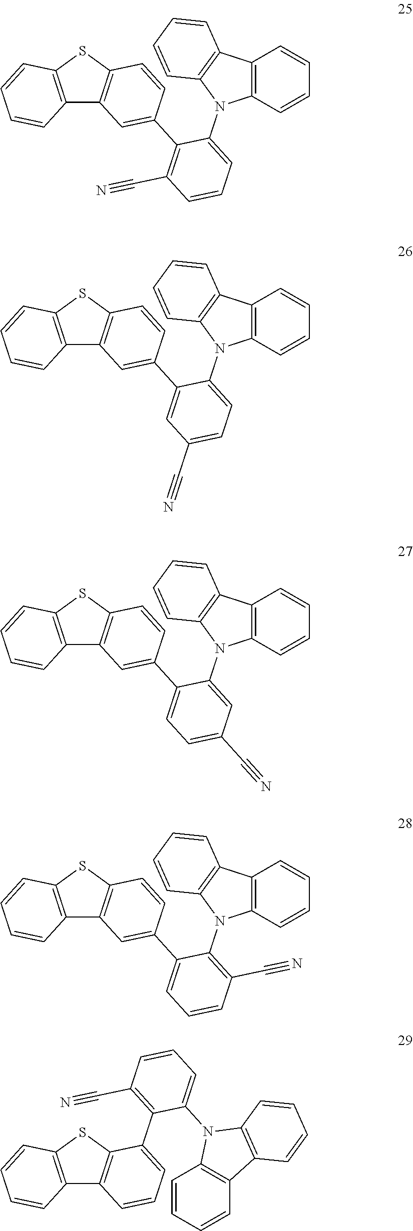

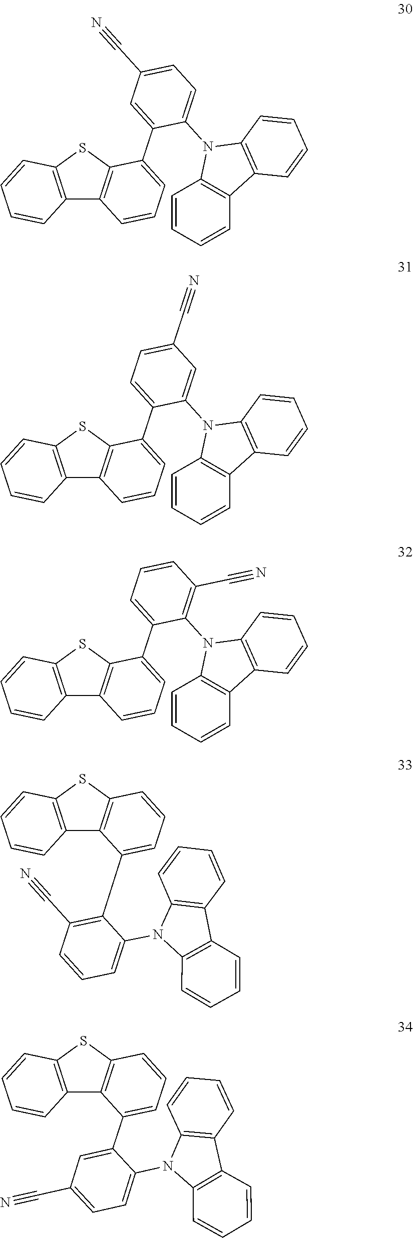

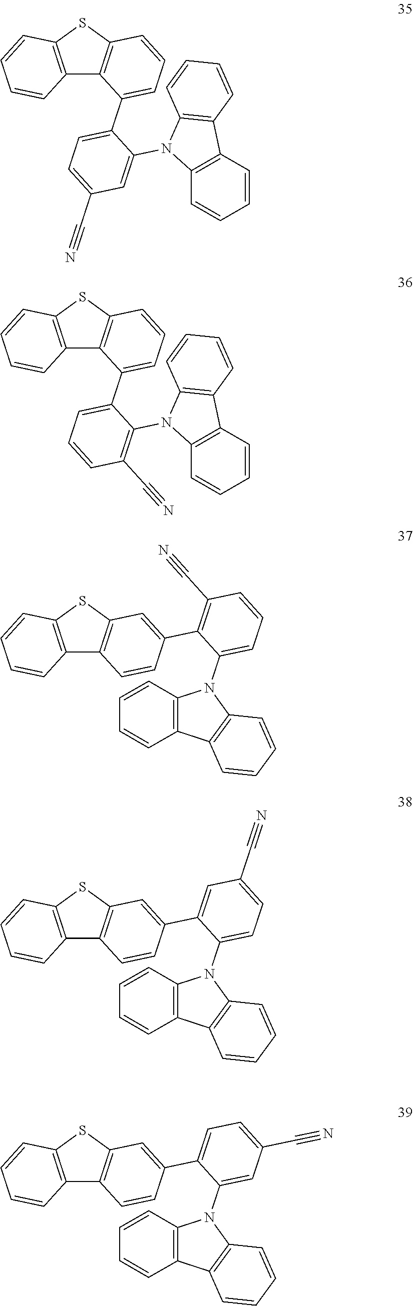

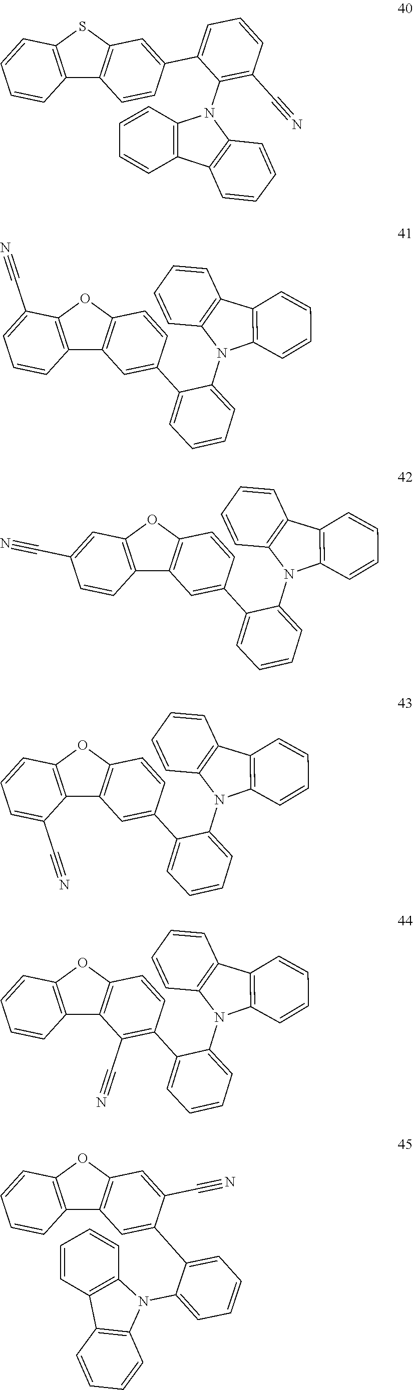

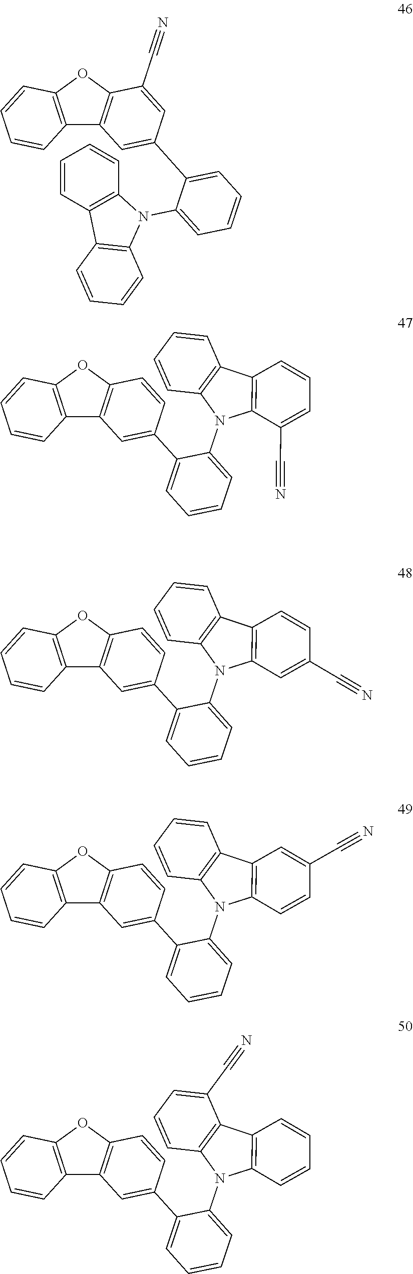 [SMILES: N#Cc1cc(-c2ccccc2-n2c3ccccc3c3ccccc32)cc2c1oc1ccccc12 ; N#Cc1cccc2c3ccccc3n(-c3ccccc3-c3ccc4oc5ccccc5c4c3)c12 ; N#Cc1ccc2c3ccccc3n(-c3ccccc3-c3ccc4oc5ccccc5c4c3)c2c1 ; N#Cc1ccc2c(c1)c1ccccc1n2-c1ccccc1-c1ccc2oc3ccccc3c2c1 ; N#Cc1cccc2c1c1ccccc1n2-c1ccccc1-c1ccc2oc3ccccc3c2c1]

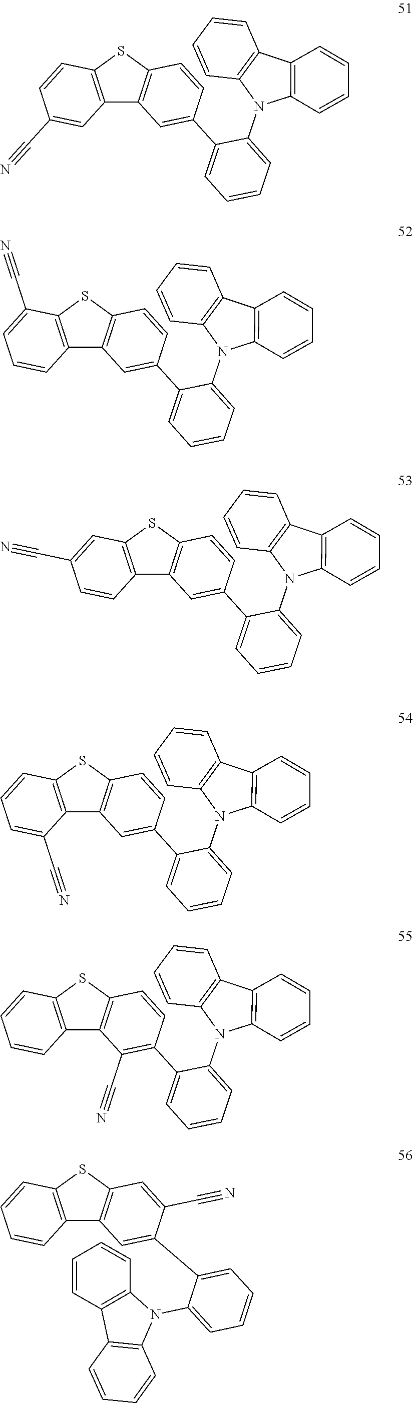

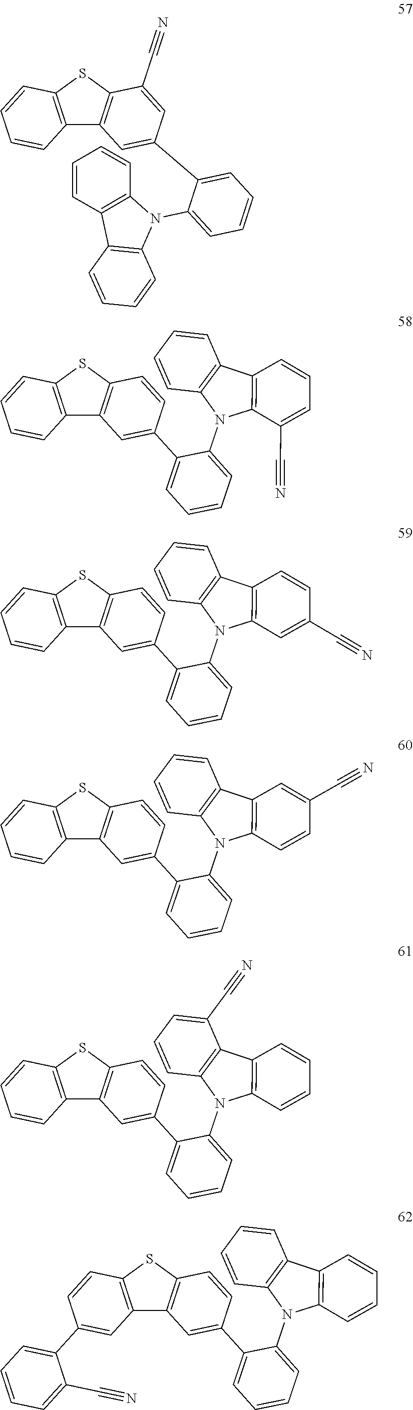

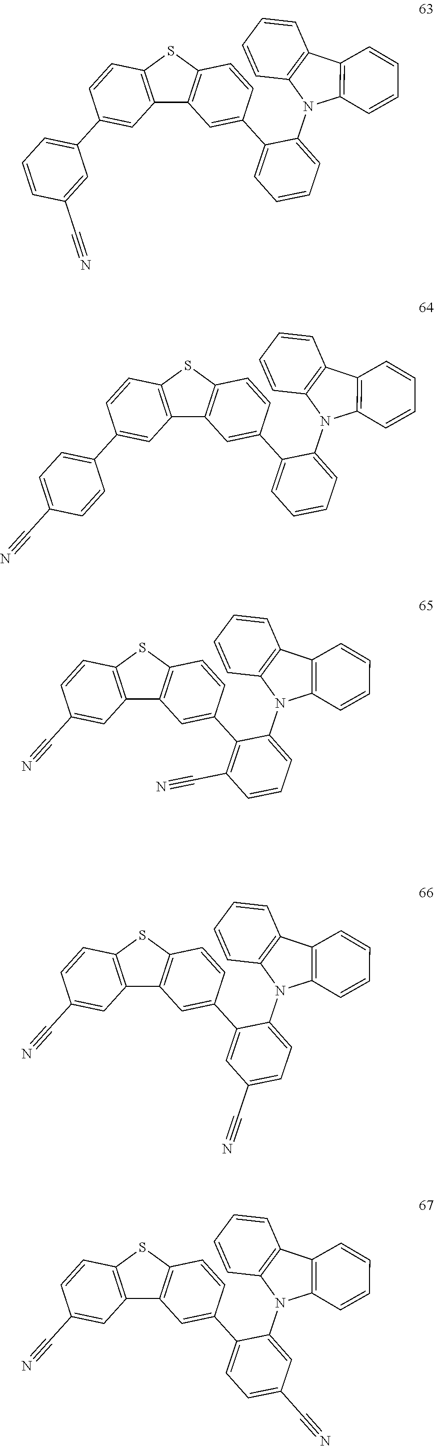 [SMILES: N#Cc1cccc(-c2ccc3sc4ccc(-c5ccccc5-n5c6ccccc6c6ccccc65)cc4c3c2)c1 ; N#Cc1ccc(-c2ccc3sc4ccc(-c5ccccc5-n5c6ccccc6c6ccccc65)cc4c3c2)cc1 ; N#Cc1ccc2sc3ccc(-c4c(C#N)cccc4-n4c5ccccc5c5ccccc54)cc3c2c1 ; N#Cc1ccc(-n2c3ccccc3c3ccccc32)c(-c2ccc3sc4ccc(C#N)cc4c3c2)c1 ; N#Cc1ccc(-c2ccc3sc4ccc(C#N)cc4c3c2)c(-n2c3ccccc3c3ccccc32)c1]

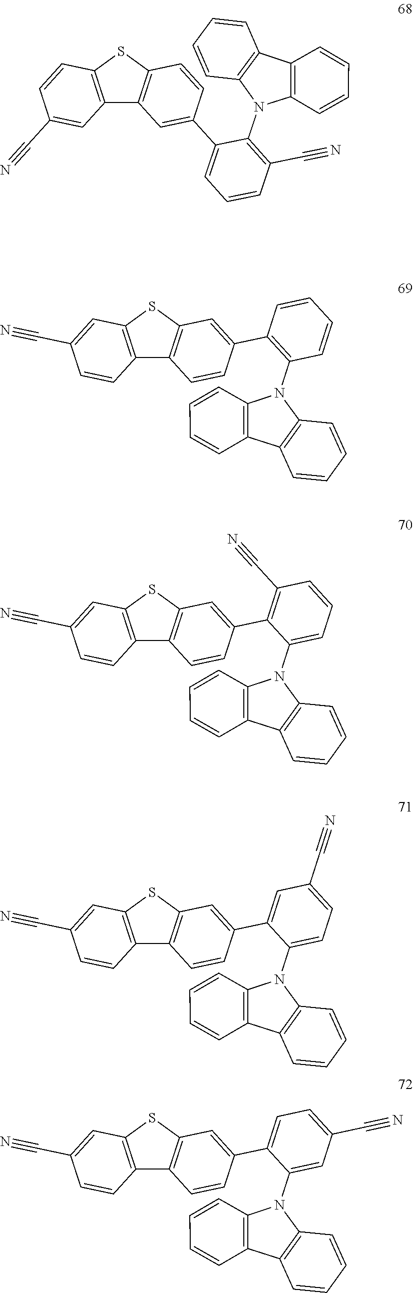

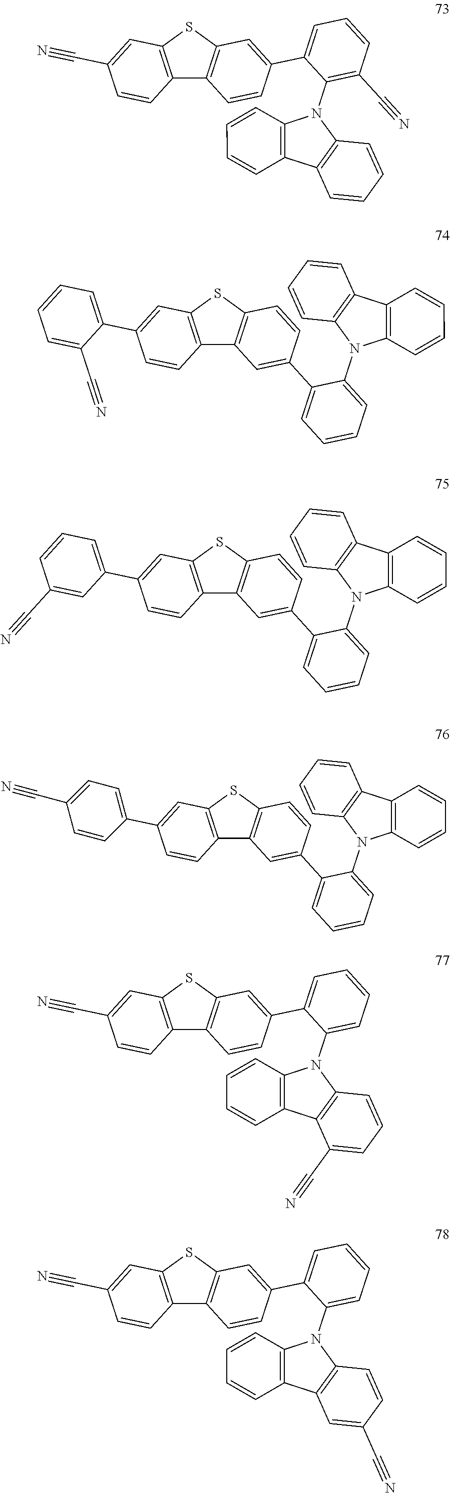

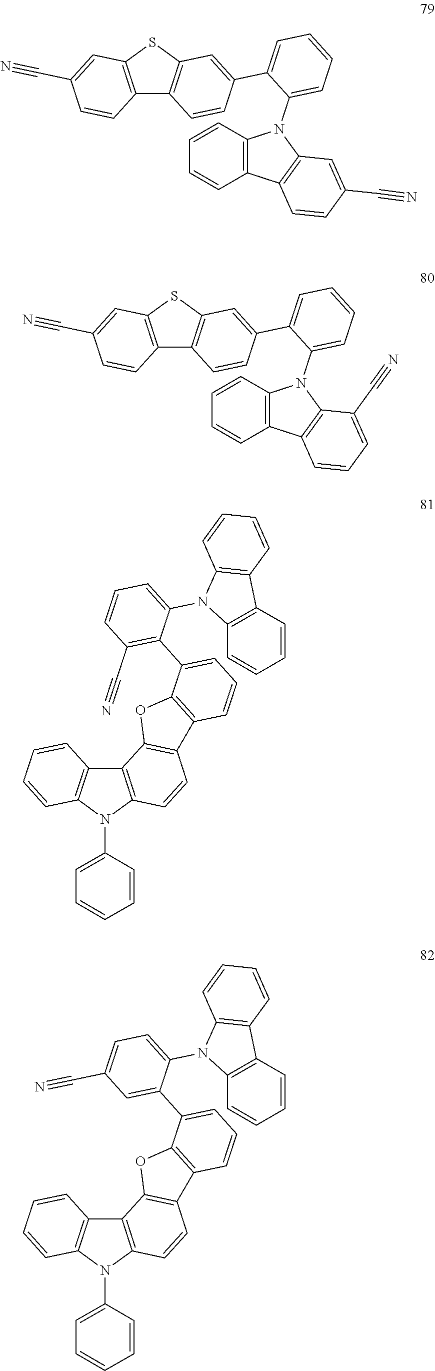 [SMILES: N#Cc1ccc2c(c1)sc1cc(-c3ccccc3-n3c4ccccc4c4ccc(C#N)cc43)ccc12 ; N#Cc1ccc2c(c1)sc1cc(-c3ccccc3-n3c4ccccc4c4cccc(C#N)c43)ccc12 ; N#Cc1cccc(-n2c3ccccc3c3ccccc32)c1-c1cccc2c1oc1c2ccc2c1c1ccccc1n2-c1ccccc1 ; N#Cc1ccc(-n2c3ccccc3c3ccccc32)c(-c2cccc3c2oc2c3ccc3c2c2ccccc2n3-c2ccccc2)c1]

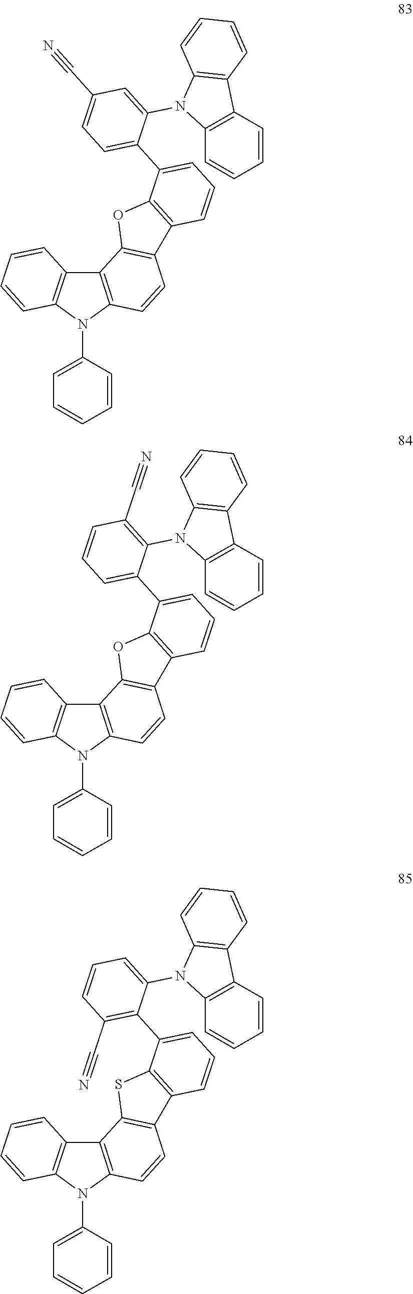 [SMILES: N#Cc1ccc(-c2cccc3c2oc2c3ccc3c2c2ccccc2n3-c2ccccc2)c(-n2c3ccccc3c3ccccc32)c1 ; N#Cc1cccc(-c2cccc3c2oc2c3ccc3c2c2ccccc2n3-c2ccccc2)c1-n1c2ccccc2c2ccccc21 ; N#Cc1cccc(-n2c3ccccc3c3ccccc32)c1-c1cccc2c1sc1c2ccc2c1c1ccccc1n2-c1ccccc1]

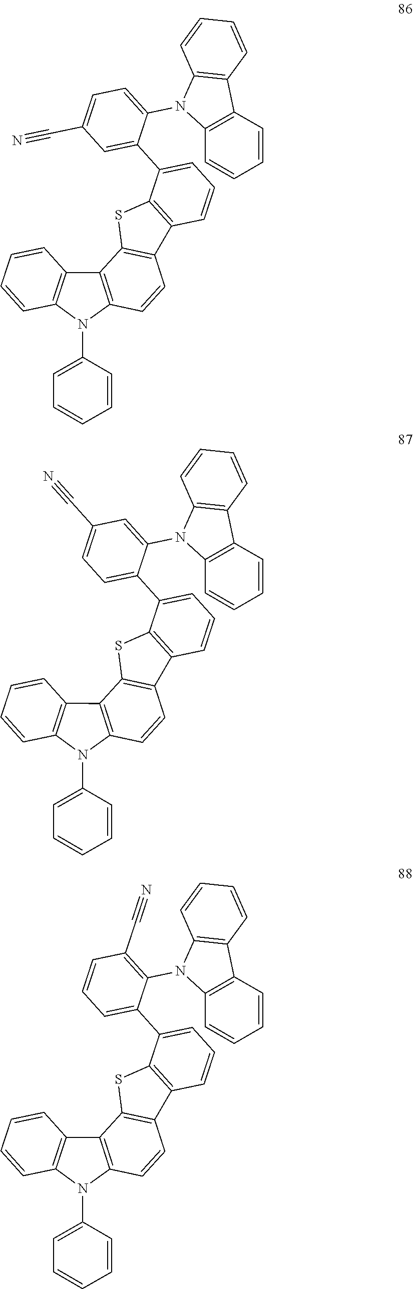 [SMILES: N#Cc1ccc(-n2c3ccccc3c3ccccc32)c(-c2cccc3c2sc2c3ccc3c2c2ccccc2n3-c2ccccc2)c1 ; N#Cc1ccc(-c2cccc3c2sc2c3ccc3c2c2ccccc2n3-c2ccccc2)c(-n2c3ccccc3c3ccccc32)c1 ; N#Cc1cccc(-c2cccc3c2sc2c3ccc3c2c2ccccc2n3-c2ccccc2)c1-n1c2ccccc2c2ccccc21]

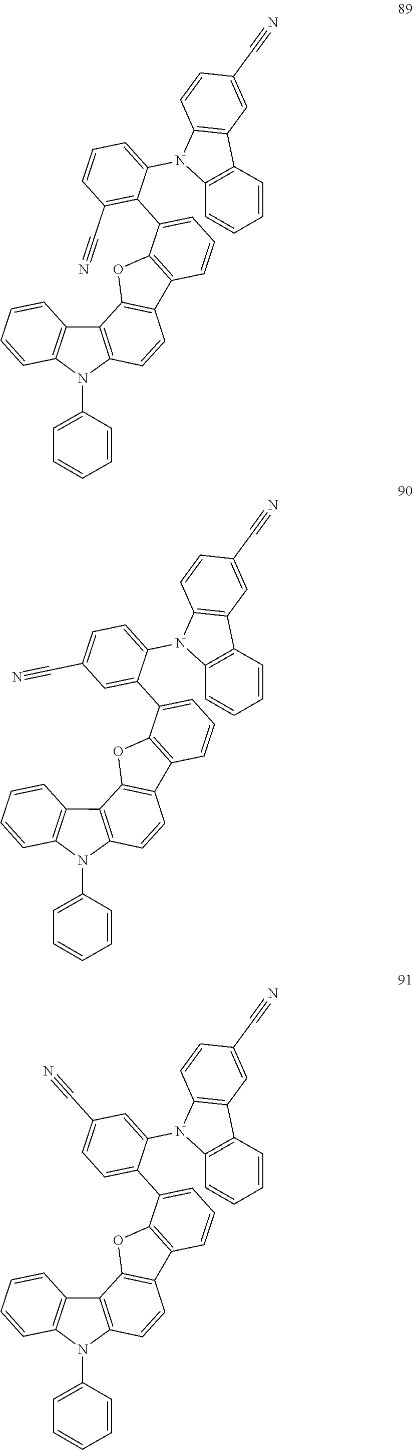 [SMILES: N#Cc1ccc2c(c1)c1ccccc1n2-c1cccc(C#N)c1-c1cccc2c1oc1c2ccc2c1c1ccccc1n2-c1ccccc1 ; N#Cc1ccc(-n2c3ccccc3c3cc(C#N)ccc32)c(-c2cccc3c2oc2c3ccc3c2c2ccccc2n3-c2ccccc2)c1 ; N#Cc1ccc(-c2cccc3c2oc2c3ccc3c2c2ccccc2n3-c2ccccc2)c(-n2c3ccccc3c3cc(C#N)ccc32)c1]

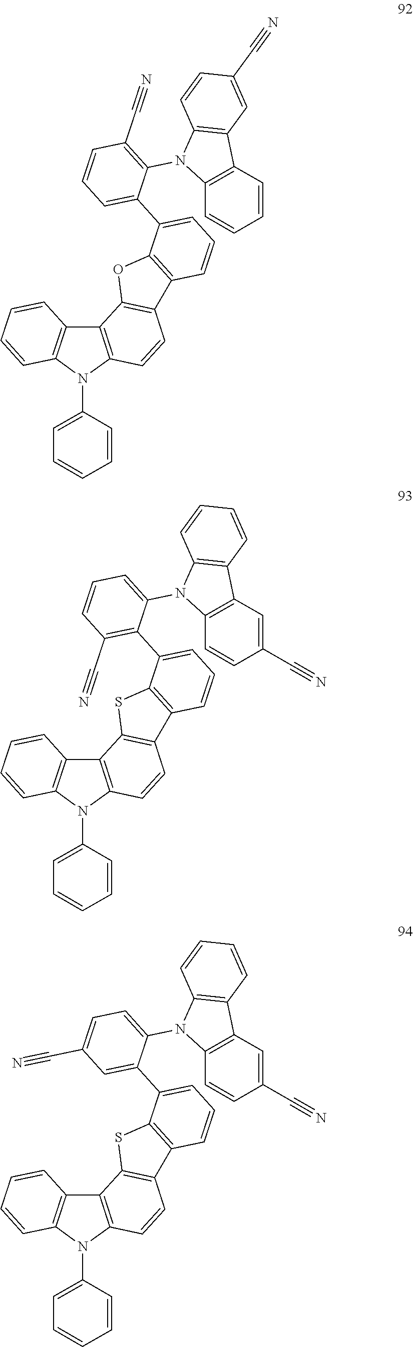 [SMILES: N#Cc1ccc2c(c1)c1ccccc1n2-c1c(C#N)cccc1-c1cccc2c1oc1c2ccc2c1c1ccccc1n2-c1ccccc1 ; N#Cc1ccc2c(c1)c1ccccc1n2-c1cccc(C#N)c1-c1cccc2c1sc1c2ccc2c1c1ccccc1n2-c1ccccc1 ; N#Cc1ccc(-n2c3ccccc3c3cc(C#N)ccc32)c(-c2cccc3c2sc2c3ccc3c2c2ccccc2n3-c2ccccc2)c1]

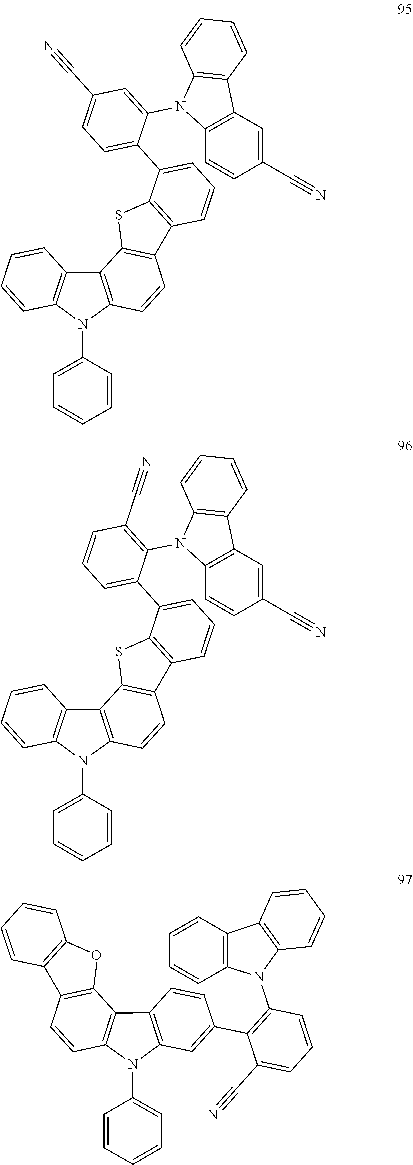 [SMILES: N#Cc1ccc(-c2cccc3c2sc2c3ccc3c2c2ccccc2n3-c2ccccc2)c(-n2c3ccccc3c3cc(C#N)ccc32)c1 ; N#Cc1ccc2c(c1)c1ccccc1n2-c1c(C#N)cccc1-c1cccc2c1sc1c2ccc2c1c1ccccc1n2-c1ccccc1 ; N#Cc1cccc(-n2c3ccccc3c3ccccc32)c1-c1ccc2c3c4oc5ccccc5c4ccc3n(-c3ccccc3)c2c1]

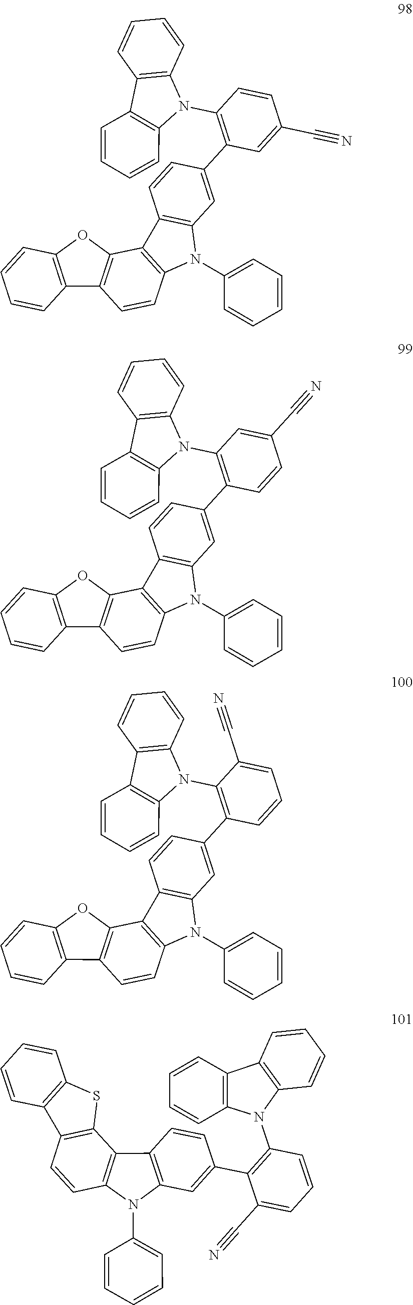

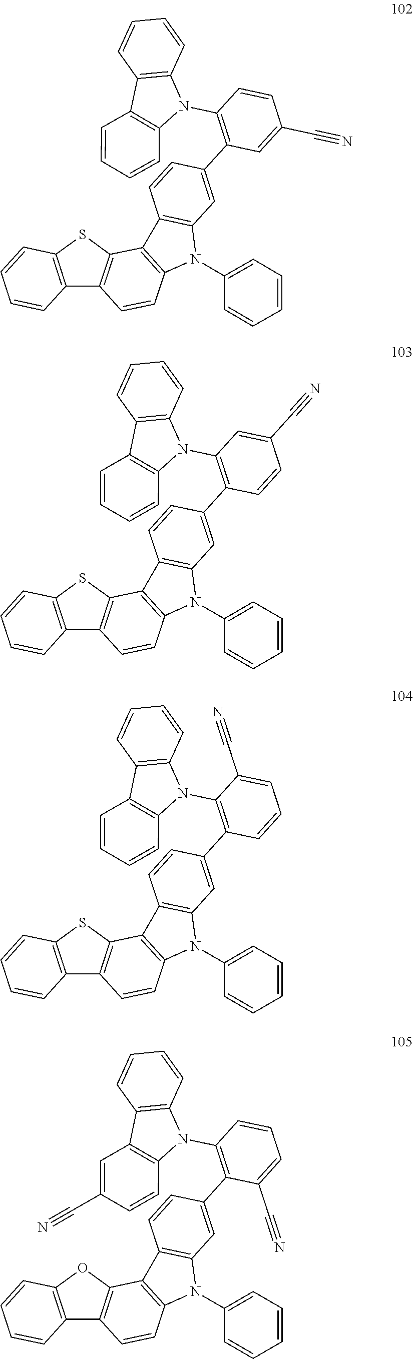 [SMILES: N#Cc1ccc(-n2c3ccccc3c3ccccc32)c(-c2ccc3c4c5sc6ccccc6c5ccc4n(-c4ccccc4)c3c2)c1 ; N#Cc1ccc(-c2ccc3c4c5sc6ccccc6c5ccc4n(-c4ccccc4)c3c2)c(-n2c3ccccc3c3ccccc32)c1 ; N#Cc1cccc(-c2ccc3c4c5sc6ccccc6c5ccc4n(-c4ccccc4)c3c2)c1-n1c2ccccc2c2ccccc21 ; N#Cc1ccc2c(c1)c1ccccc1n2-c1cccc(C#N)c1-c1ccc2c3c4oc5ccccc5c4ccc3n(-c3ccccc3)c2c1]

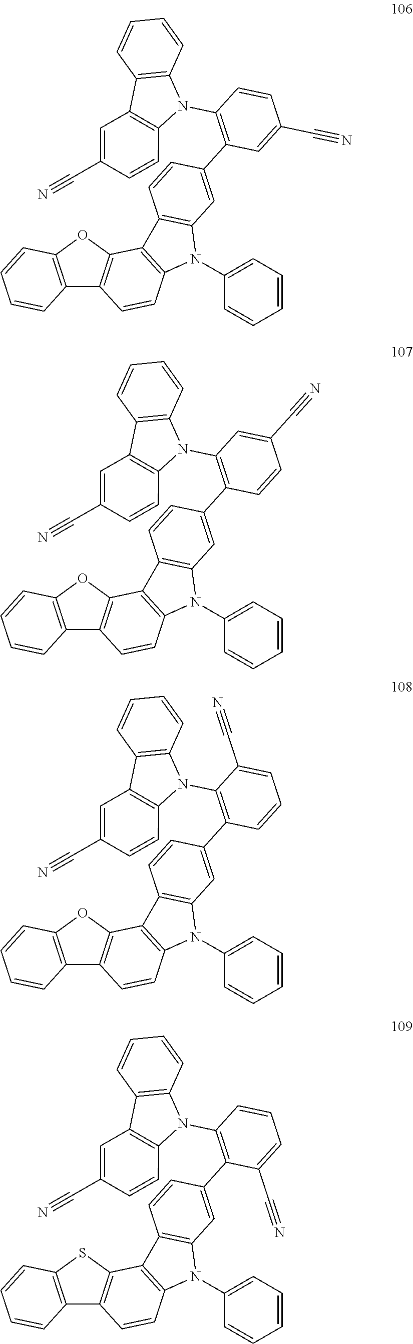

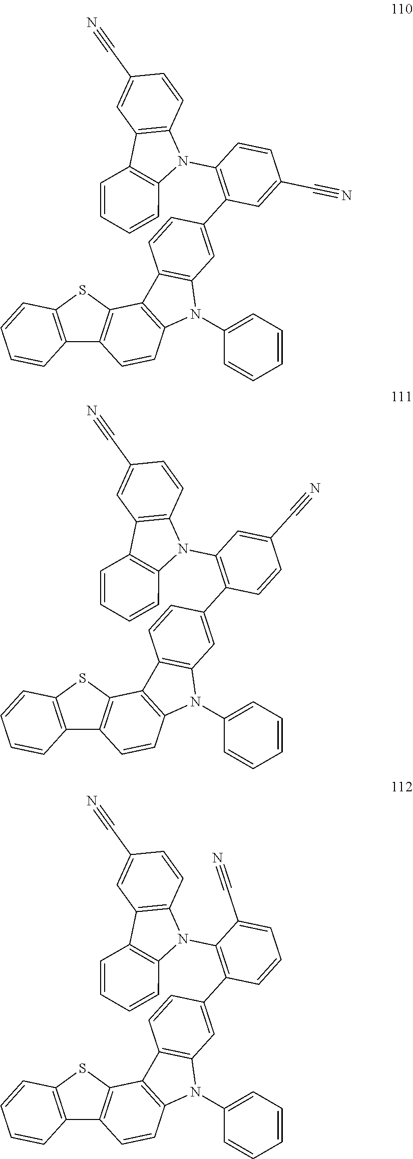 [SMILES: N#Cc1ccc(-n2c3ccccc3c3cc(C#N)ccc32)c(-c2ccc3c4c5sc6ccccc6c5ccc4n(-c4ccccc4)c3c2)c1 ; N#Cc1ccc(-c2ccc3c4c5sc6ccccc6c5ccc4n(-c4ccccc4)c3c2)c(-n2c3ccccc3c3cc(C#N)ccc32)c1 ; N#Cc1ccc2c(c1)c1ccccc1n2-c1c(C#N)cccc1-c1ccc2c3c4sc5ccccc5c4ccc3n(-c3ccccc3)c2c1]

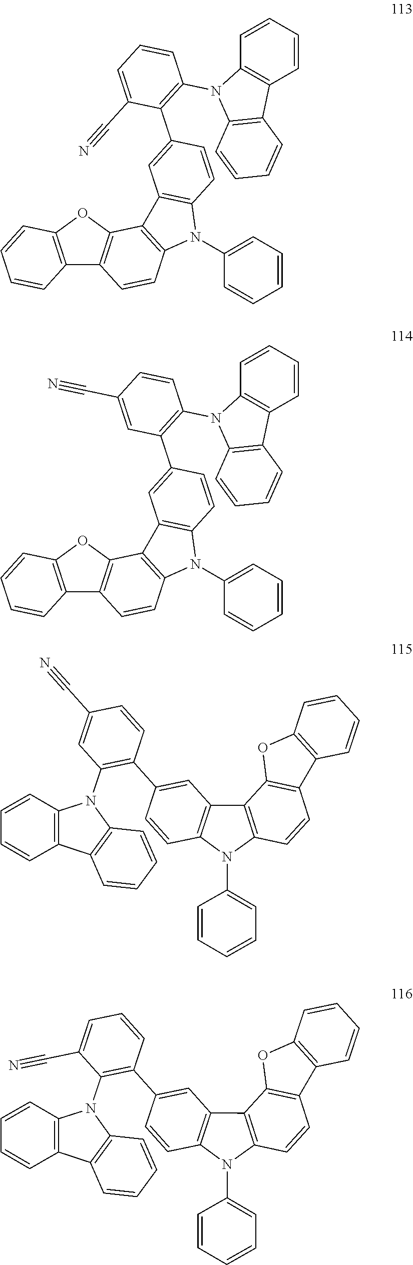

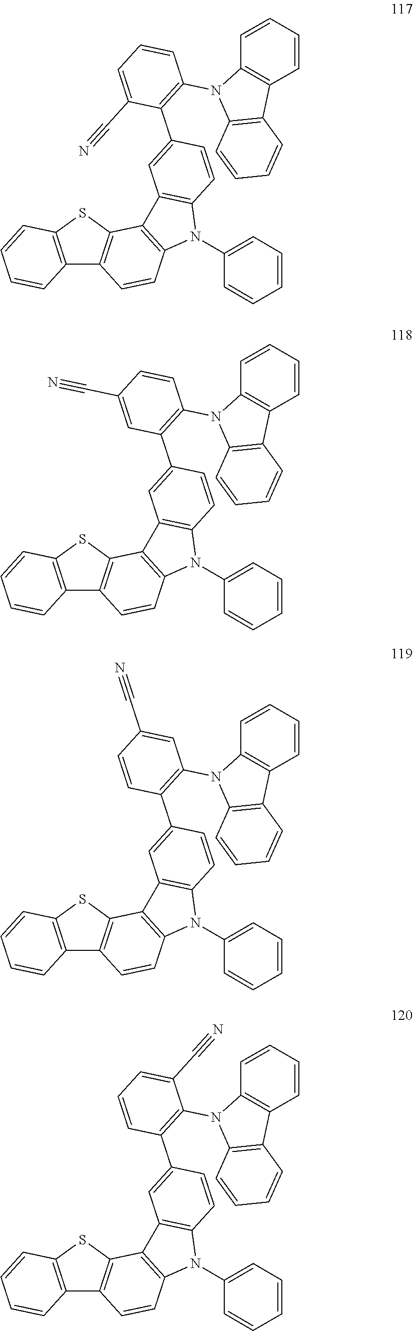

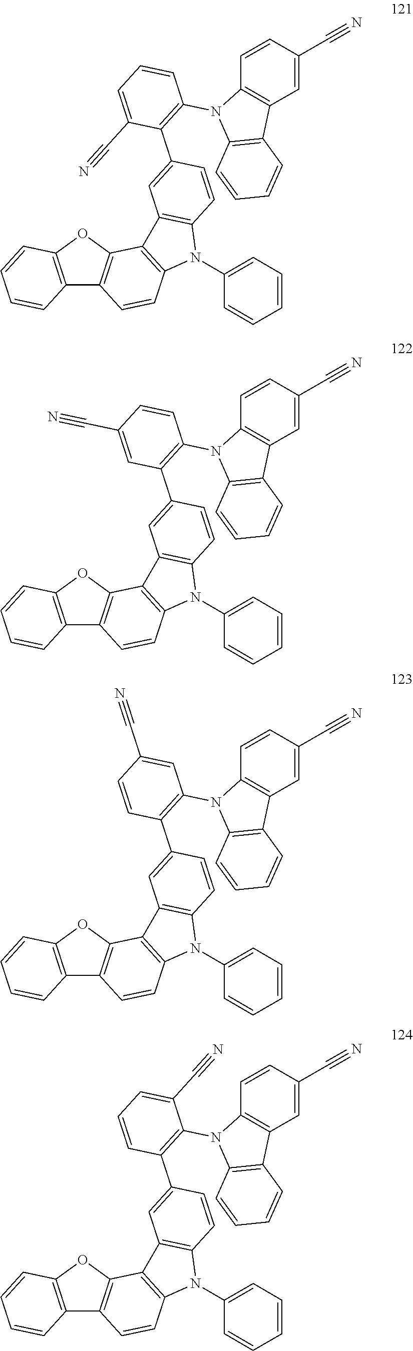

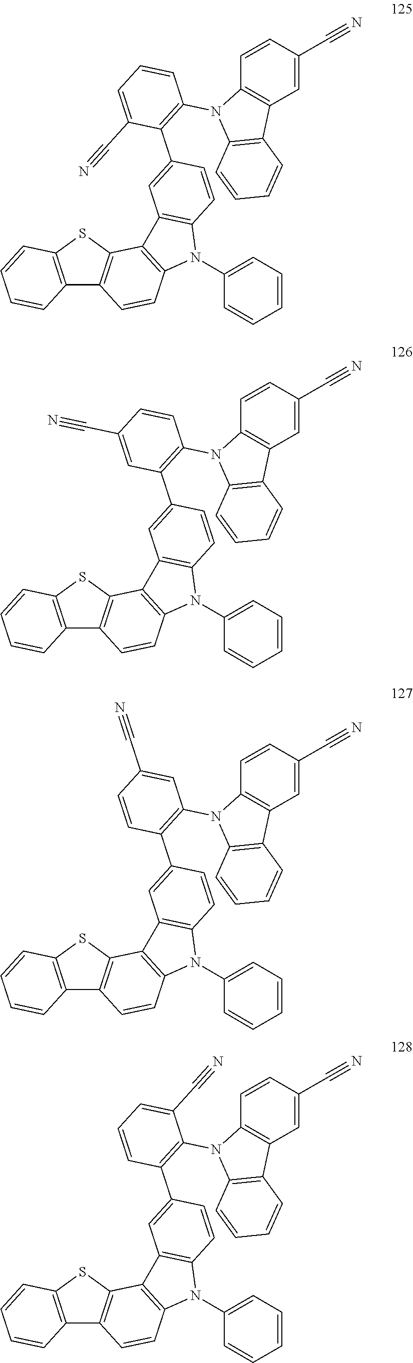 [SMILES: N#Cc1ccc2c(c1)c1ccccc1n2-c1cccc(C#N)c1-c1ccc2c(c1)c1c3sc4ccccc4c3ccc1n2-c1ccccc1 ; N#Cc1ccc(-n2c3ccccc3c3cc(C#N)ccc32)c(-c2ccc3c(c2)c2c4sc5ccccc5c4ccc2n3-c2ccccc2)c1 ; N#Cc1ccc(-c2ccc3c(c2)c2c4sc5ccccc5c4ccc2n3-c2ccccc2)c(-n2c3ccccc3c3cc(C#N)ccc32)c1 ; N#Cc1ccc2c(c1)c1ccccc1n2-c1c(C#N)cccc1-c1ccc2c(c1)c1c3sc4ccccc4c3ccc1n2-c1ccccc1]

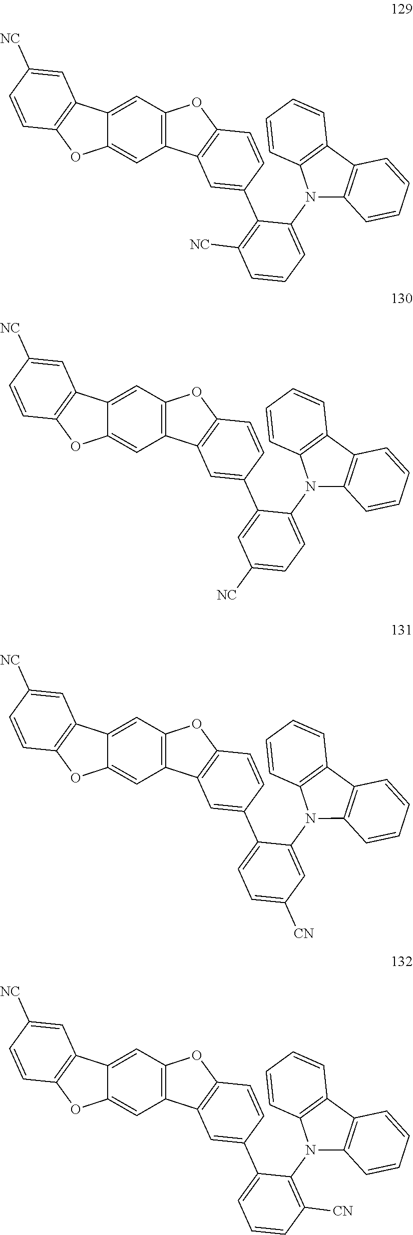 [SMILES: N#Cc1ccc2oc3cc4c(cc3c2c1)oc1ccc(-c2c(C#N)cccc2-n2c3ccccc3c3ccccc32)cc14 ; N#Cc1ccc(-n2c3ccccc3c3ccccc32)c(-c2ccc3oc4cc5c(cc4c3c2)oc2ccc(C#N)cc25)c1 ; N#Cc1ccc(-c2ccc3oc4cc5c(cc4c3c2)oc2ccc(C#N)cc25)c(-n2c3ccccc3c3ccccc32)c1 ; N#Cc1ccc2oc3cc4c(cc3c2c1)oc1ccc(-c2cccc(C#N)c2-n2c3ccccc3c3ccccc32)cc14]

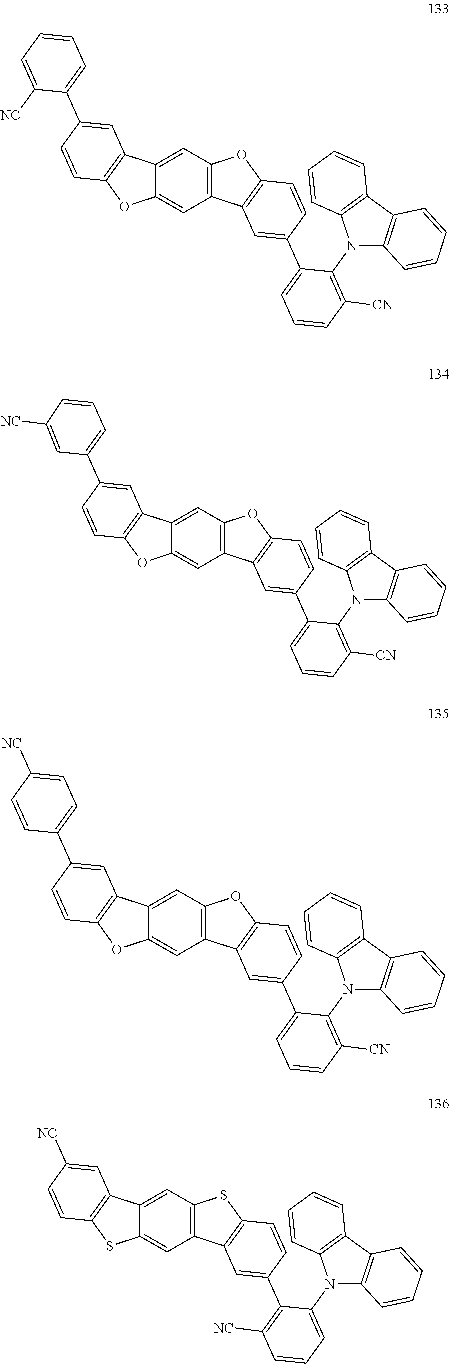 [SMILES: N#Cc1ccccc1-c1ccc2oc3cc4c(cc3c2c1)oc1ccc(-c2cccc(C#N)c2-n2c3ccccc3c3ccccc32)cc14 ; N#Cc1cccc(-c2ccc3oc4cc5c(cc4c3c2)oc2ccc(-c3cccc(C#N)c3-n3c4ccccc4c4ccccc43)cc25)c1 ; N#Cc1ccc(-c2ccc3oc4cc5c(cc4c3c2)oc2ccc(-c3cccc(C#N)c3-n3c4ccccc4c4ccccc43)cc25)cc1 ; N#Cc1ccc2sc3cc4c(cc3c2c1)sc1ccc(-c2c(C#N)cccc2-n2c3ccccc3c3ccccc32)cc14]

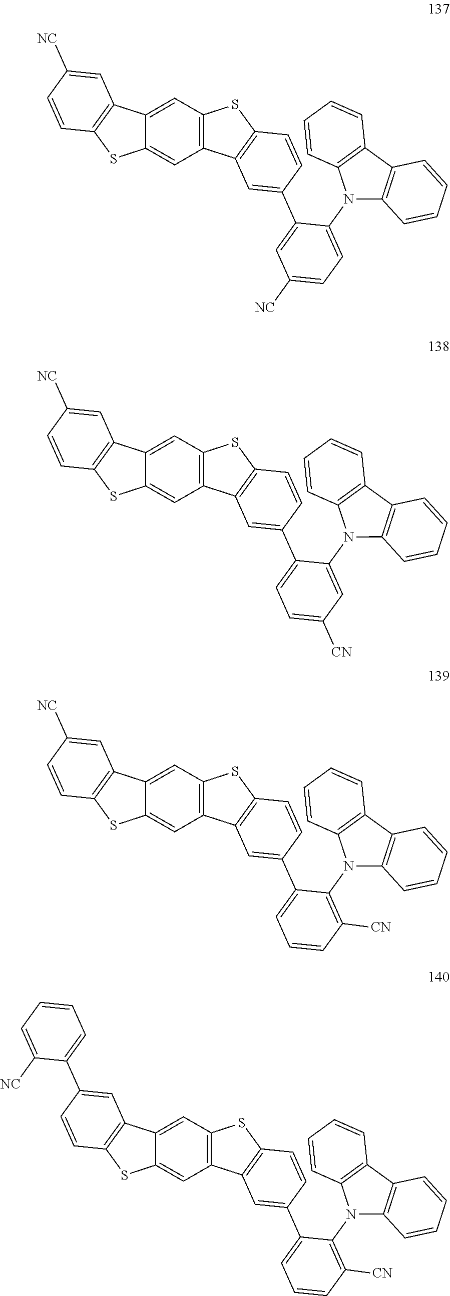 [SMILES: N#Cc1ccc(-n2c3ccccc3c3ccccc32)c(-c2ccc3sc4cc5c(cc4c3c2)sc2ccc(C#N)cc25)c1 ; N#Cc1ccc(-c2ccc3sc4cc5c(cc4c3c2)sc2ccc(C#N)cc25)c(-n2c3ccccc3c3ccccc32)c1 ; N#Cc1ccc2sc3cc4c(cc3c2c1)sc1ccc(-c2cccc(C#N)c2-n2c3ccccc3c3ccccc32)cc14 ; N#Cc1ccccc1-c1ccc2sc3cc4c(cc3c2c1)sc1ccc(-c2cccc(C#N)c2-n2c3ccccc3c3ccccc32)cc14]

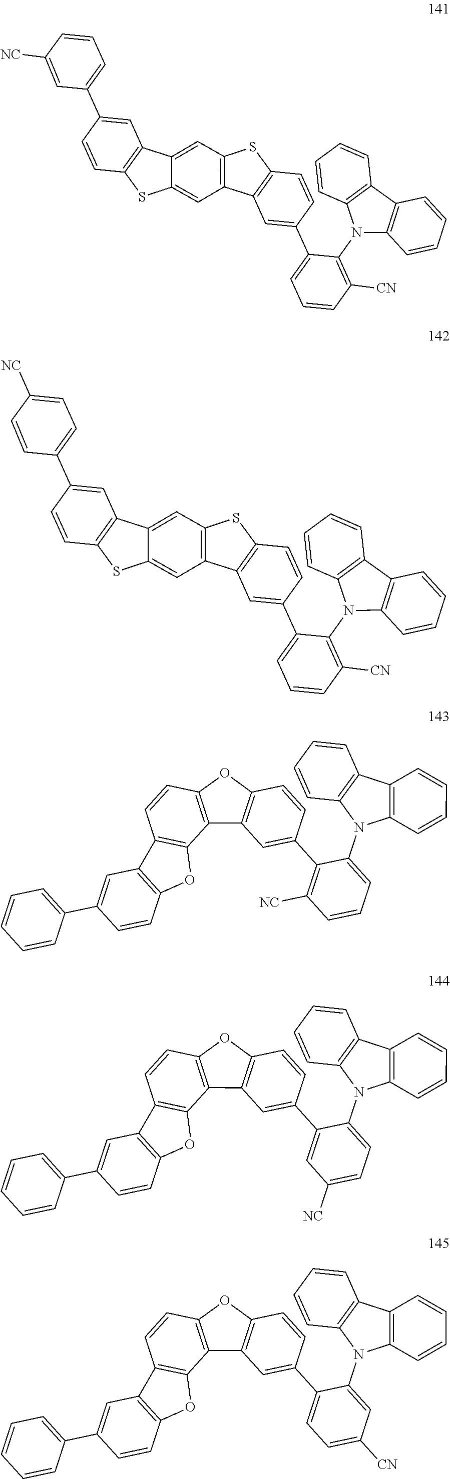 [SMILES: N#Cc1cccc(-c2ccc3sc4cc5c(cc4c3c2)sc2ccc(-c3cccc(C#N)c3-n3c4ccccc4c4ccccc43)cc25)c1 ; N#Cc1ccc(-c2ccc3sc4cc5c(cc4c3c2)sc2ccc(-c3cccc(C#N)c3-n3c4ccccc4c4ccccc43)cc25)cc1 ; N#Cc1cccc(-n2c3ccccc3c3ccccc32)c1-c1ccc2oc3ccc4c5cc(-c6ccccc6)ccc5oc4c3c2c1 ; N#Cc1ccc(-n2c3ccccc3c3ccccc32)c(-c2ccc3oc4ccc5c6cc(-c7ccccc7)ccc6oc5c4c3c2)c1 ; N#Cc1ccc(-c2ccc3oc4ccc5c6cc(-c7ccccc7)ccc6oc5c4c3c2)c(-n2c3ccccc3c3ccccc32)c1]

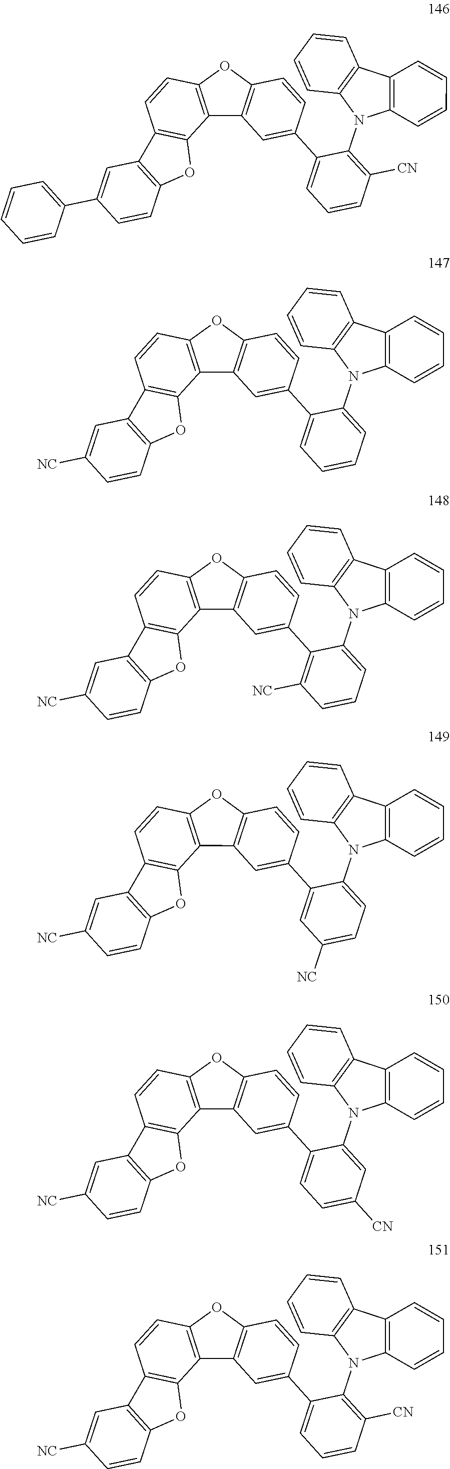 [SMILES: N#Cc1cccc(-c2ccc3oc4ccc5c6cc(-c7ccccc7)ccc6oc5c4c3c2)c1-n1c2ccccc2c2ccccc21 ; N#Cc1ccc2oc3c(ccc4oc5ccc(-c6ccccc6-n6c7ccccc7c7ccccc76)cc5c43)c2c1 ; N#Cc1ccc2oc3c(ccc4oc5ccc(-c6c(C#N)cccc6-n6c7ccccc7c7ccccc76)cc5c43)c2c1 ; N#Cc1ccc(-n2c3ccccc3c3ccccc32)c(-c2ccc3oc4ccc5c6cc(C#N)ccc6oc5c4c3c2)c1 ; N#Cc1ccc(-c2ccc3oc4ccc5c6cc(C#N)ccc6oc5c4c3c2)c(-n2c3ccccc3c3ccccc32)c1 ; N#Cc1ccc2oc3c(ccc4oc5ccc(-c6cccc(C#N)c6-n6c7ccccc7c7ccccc76)cc5c43)c2c1]

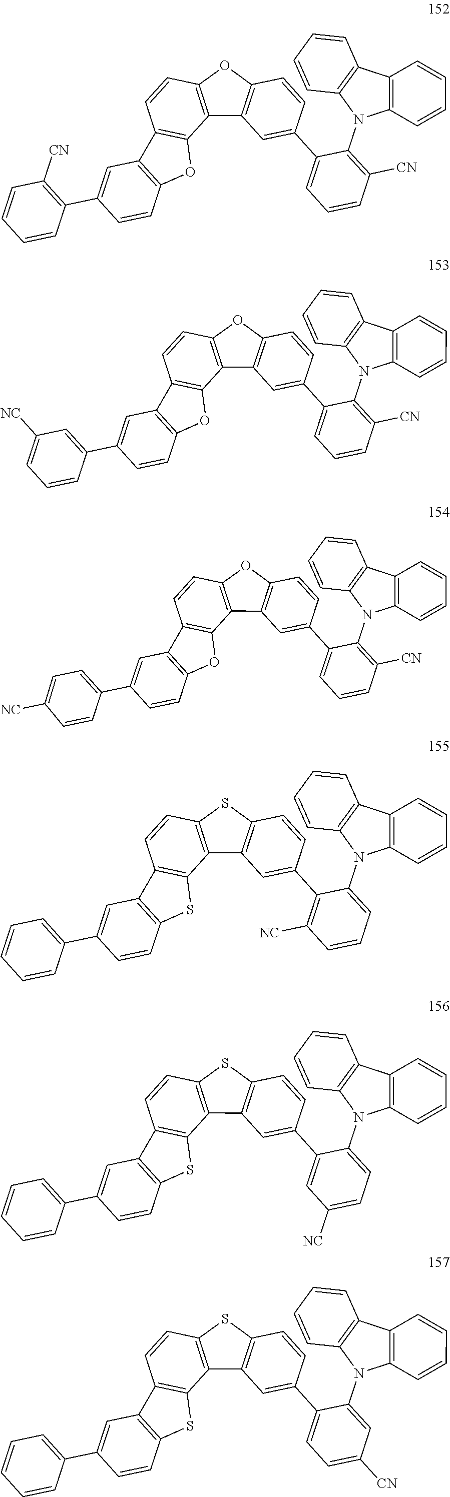 [SMILES: N#Cc1ccccc1-c1ccc2oc3c(ccc4oc5ccc(-c6cccc(C#N)c6-n6c7ccccc7c7ccccc76)cc5c43)c2c1 ; N#Cc1cccc(-c2ccc3oc4c(ccc5oc6ccc(-c7cccc(C#N)c7-n7c8ccccc8c8ccccc87)cc6c54)c3c2)c1 ; N#Cc1ccc(-c2ccc3oc4c(ccc5oc6ccc(-c7cccc(C#N)c7-n7c8ccccc8c8ccccc87)cc6c54)c3c2)cc1 ; N#Cc1cccc(-n2c3ccccc3c3ccccc32)c1-c1ccc2sc3ccc4c5cc(-c6ccccc6)ccc5sc4c3c2c1 ; N#Cc1ccc(-n2c3ccccc3c3ccccc32)c(-c2ccc3sc4ccc5c6cc(-c7ccccc7)ccc6sc5c4c3c2)c1 ; N#Cc1ccc(-c2ccc3sc4ccc5c6cc(-c7ccccc7)ccc6sc5c4c3c2)c(-n2c3ccccc3c3ccccc32)c1]

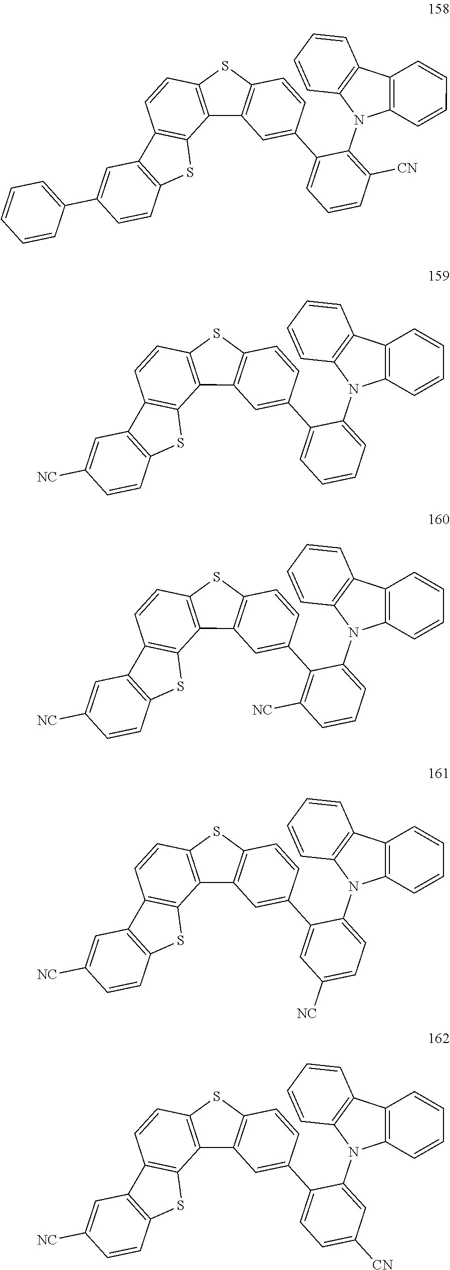 [SMILES: N#Cc1cccc(-c2ccc3sc4ccc5c6cc(-c7ccccc7)ccc6sc5c4c3c2)c1-n1c2ccccc2c2ccccc21 ; N#Cc1ccc2sc3c(ccc4sc5ccc(-c6ccccc6-n6c7ccccc7c7ccccc76)cc5c43)c2c1 ; N#Cc1ccc2sc3c(ccc4sc5ccc(-c6c(C#N)cccc6-n6c7ccccc7c7ccccc76)cc5c43)c2c1 ; N#Cc1ccc(-n2c3ccccc3c3ccccc32)c(-c2ccc3sc4ccc5c6cc(C#N)ccc6sc5c4c3c2)c1 ; N#Cc1ccc(-c2ccc3sc4ccc5c6cc(C#N)ccc6sc5c4c3c2)c(-n2c3ccccc3c3ccccc32)c1]

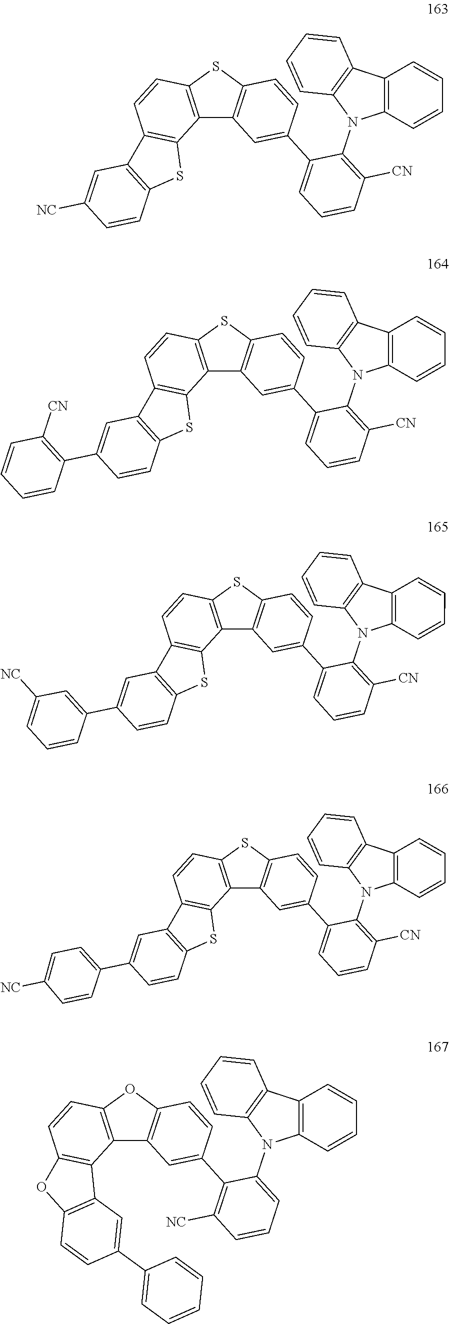 [SMILES: N#Cc1ccc2sc3c(ccc4sc5ccc(-c6cccc(C#N)c6-n6c7ccccc7c7ccccc76)cc5c43)c2c1 ; N#Cc1ccccc1-c1ccc2sc3c(ccc4sc5ccc(-c6cccc(C#N)c6-n6c7ccccc7c7ccccc76)cc5c43)c2c1 ; N#Cc1cccc(-c2ccc3sc4c(ccc5sc6ccc(-c7cccc(C#N)c7-n7c8ccccc8c8ccccc87)cc6c54)c3c2)c1 ; N#Cc1ccc(-c2ccc3sc4c(ccc5sc6ccc(-c7cccc(C#N)c7-n7c8ccccc8c8ccccc87)cc6c54)c3c2)cc1 ; N#Cc1cccc(-n2c3ccccc3c3ccccc32)c1-c1ccc2oc3ccc4oc5ccc(-c6ccccc6)cc5c4c3c2c1]

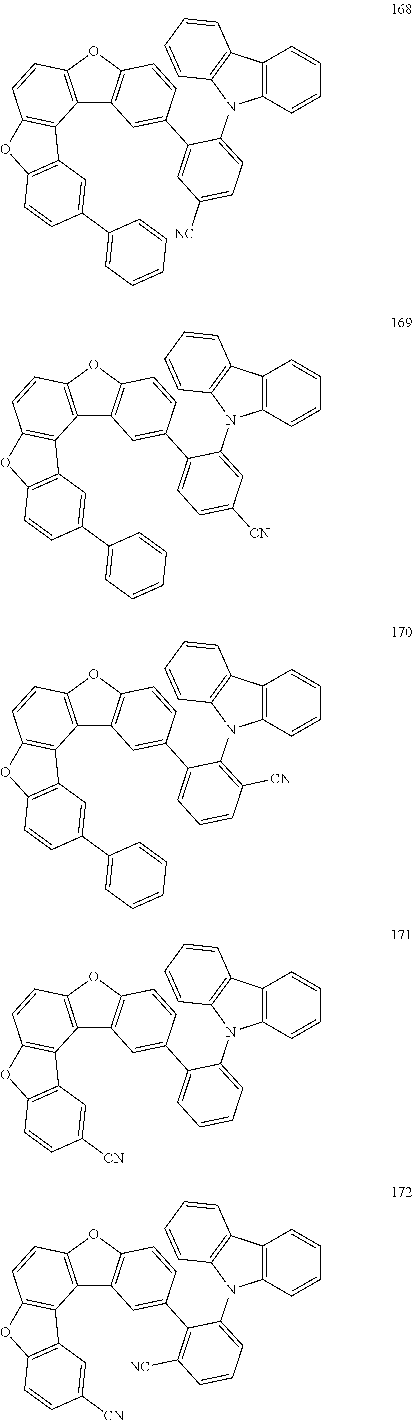 [SMILES: N#Cc1ccc(-n2c3ccccc3c3ccccc32)c(-c2ccc3oc4ccc5oc6ccc(-c7ccccc7)cc6c5c4c3c2)c1 ; N#Cc1ccc(-c2ccc3oc4ccc5oc6ccc(-c7ccccc7)cc6c5c4c3c2)c(-n2c3ccccc3c3ccccc32)c1 ; N#Cc1cccc(-c2ccc3oc4ccc5oc6ccc(-c7ccccc7)cc6c5c4c3c2)c1-n1c2ccccc2c2ccccc21 ; N#Cc1ccc2oc3ccc4oc5ccc(-c6ccccc6-n6c7ccccc7c7ccccc76)cc5c4c3c2c1 ; N#Cc1ccc2oc3ccc4oc5ccc(-c6c(C#N)cccc6-n6c7ccccc7c7ccccc76)cc5c4c3c2c1]

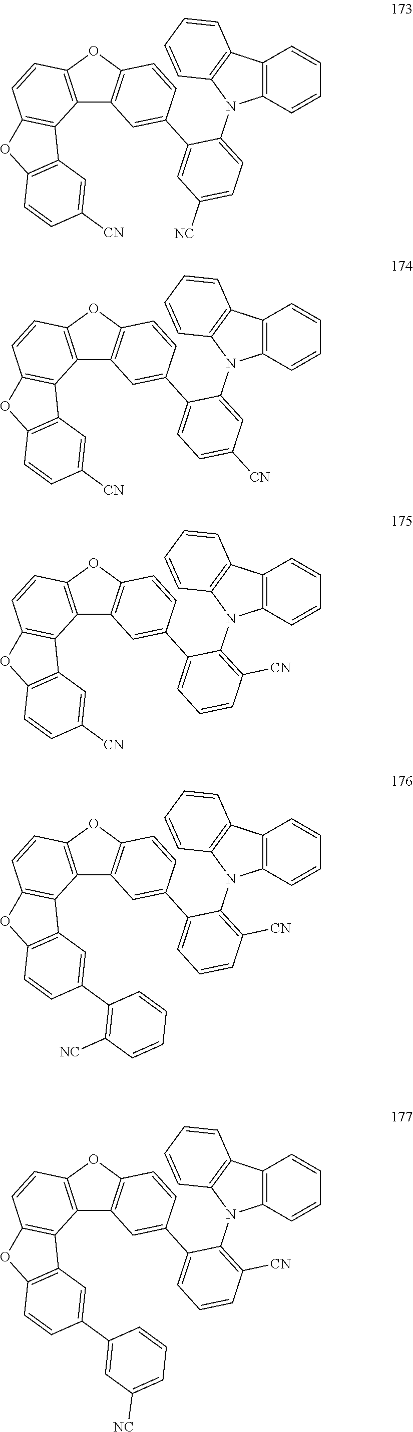

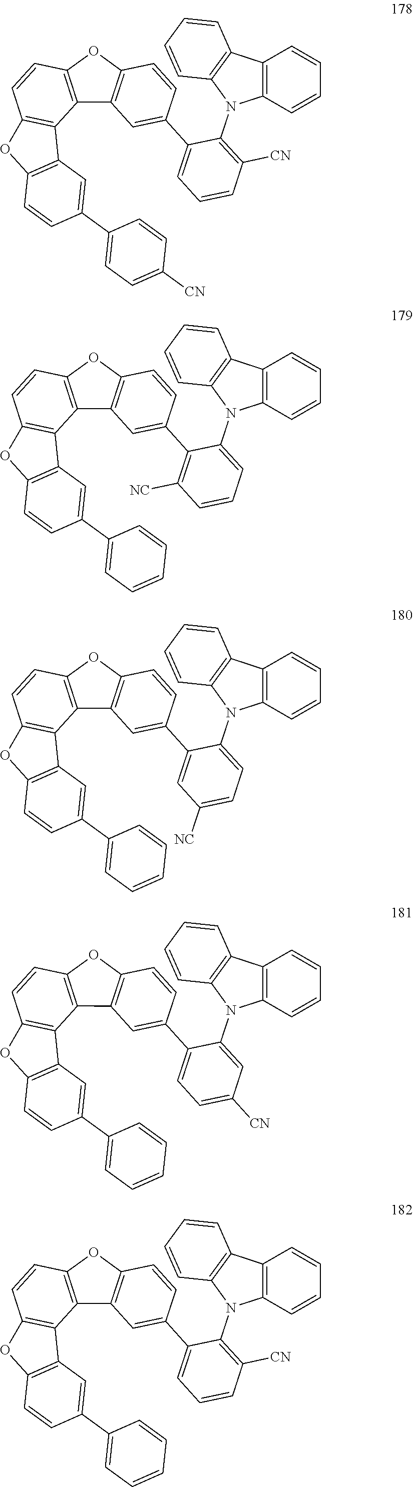

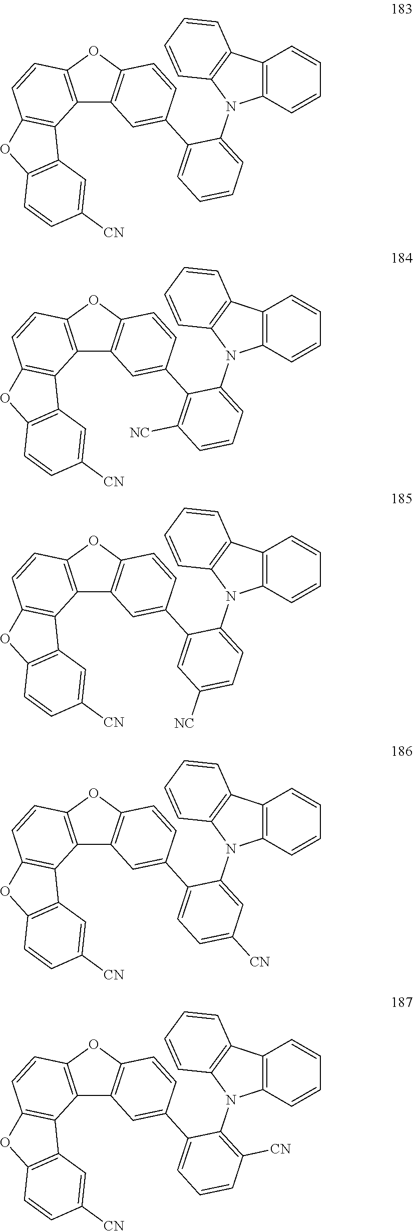

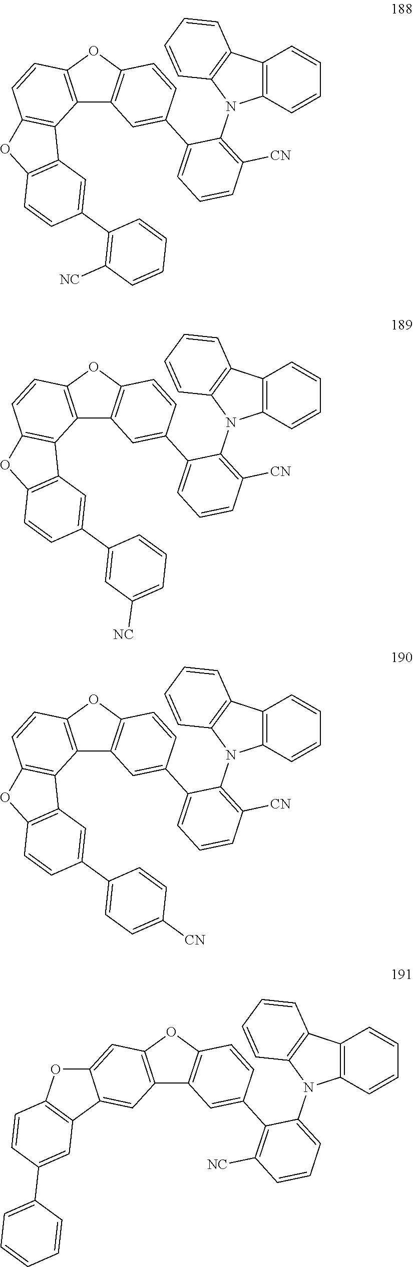 [SMILES: N#Cc1ccccc1-c1ccc2oc3ccc4oc5ccc(-c6cccc(C#N)c6-n6c7ccccc7c7ccccc76)cc5c4c3c2c1 ; N#Cc1cccc(-c2ccc3oc4ccc5oc6ccc(-c7cccc(C#N)c7-n7c8ccccc8c8ccccc87)cc6c5c4c3c2)c1 ; N#Cc1ccc(-c2ccc3oc4ccc5oc6ccc(-c7cccc(C#N)c7-n7c8ccccc8c8ccccc87)cc6c5c4c3c2)cc1 ; N#Cc1cccc(-n2c3ccccc3c3ccccc32)c1-c1ccc2oc3cc4oc5ccc(-c6ccccc6)cc5c4cc3c2c1]

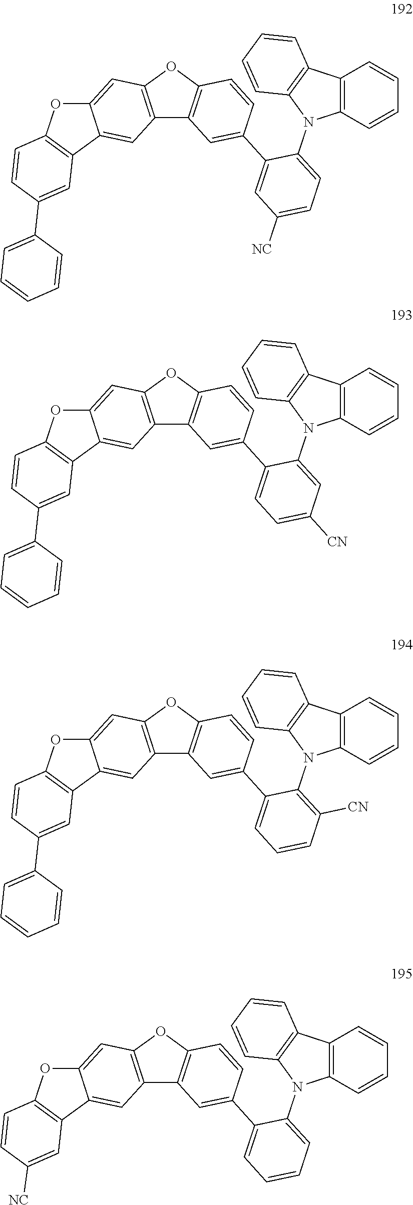 [SMILES: N#Cc1ccc(-n2c3ccccc3c3ccccc32)c(-c2ccc3oc4cc5oc6ccc(-c7ccccc7)cc6c5cc4c3c2)c1 ; N#Cc1ccc(-c2ccc3oc4cc5oc6ccc(-c7ccccc7)cc6c5cc4c3c2)c(-n2c3ccccc3c3ccccc32)c1 ; N#Cc1cccc(-c2ccc3oc4cc5oc6ccc(-c7ccccc7)cc6c5cc4c3c2)c1-n1c2ccccc2c2ccccc21 ; N#Cc1ccc2oc3cc4oc5ccc(-c6ccccc6-n6c7ccccc7c7ccccc76)cc5c4cc3c2c1]

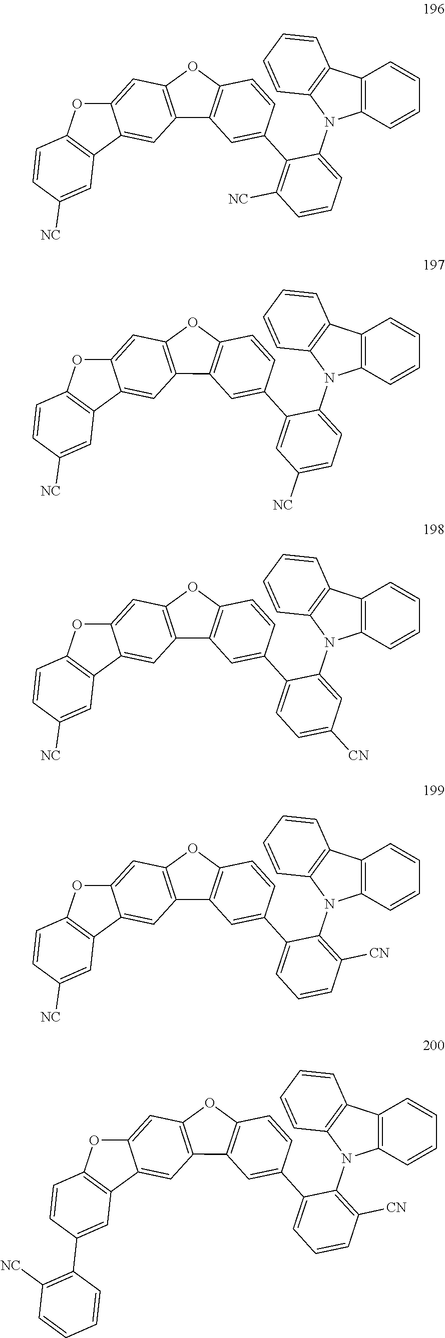 [SMILES: N#Cc1ccc2oc3cc4oc5ccc(-c6c(C#N)cccc6-n6c7ccccc7c7ccccc76)cc5c4cc3c2c1 ; N#Cc1ccc(-n2c3ccccc3c3ccccc32)c(-c2ccc3oc4cc5oc6ccc(C#N)cc6c5cc4c3c2)c1 ; N#Cc1ccc(-c2ccc3oc4cc5oc6ccc(C#N)cc6c5cc4c3c2)c(-n2c3ccccc3c3ccccc32)c1 ; N#Cc1ccc2oc3cc4oc5ccc(-c6cccc(C#N)c6-n6c7ccccc7c7ccccc76)cc5c4cc3c2c1 ; N#Cc1ccccc1-c1ccc2oc3cc4oc5ccc(-c6cccc(C#N)c6-n6c7ccccc7c7ccccc76)cc5c4cc3c2c1]

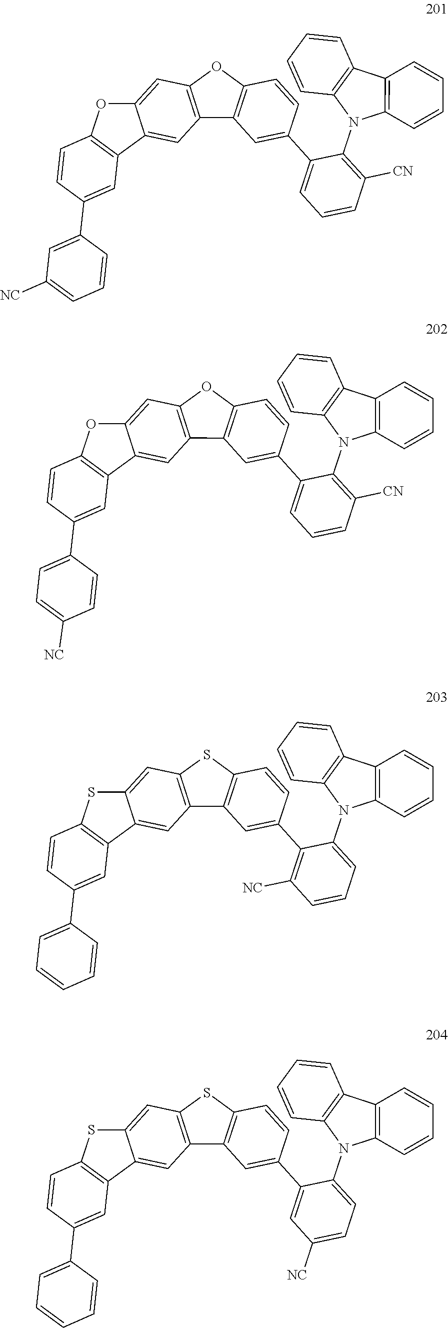 [SMILES: N#Cc1cccc(-c2ccc3oc4cc5oc6ccc(-c7cccc(C#N)c7-n7c8ccccc8c8ccccc87)cc6c5cc4c3c2)c1 ; N#Cc1ccc(-c2ccc3oc4cc5oc6ccc(-c7cccc(C#N)c7-n7c8ccccc8c8ccccc87)cc6c5cc4c3c2)cc1 ; N#Cc1cccc(-n2c3ccccc3c3ccccc32)c1-c1ccc2sc3cc4sc5ccc(-c6ccccc6)cc5c4cc3c2c1 ; N#Cc1ccc(-n2c3ccccc3c3ccccc32)c(-c2ccc3sc4cc5sc6ccc(-c7ccccc7)cc6c5cc4c3c2)c1]

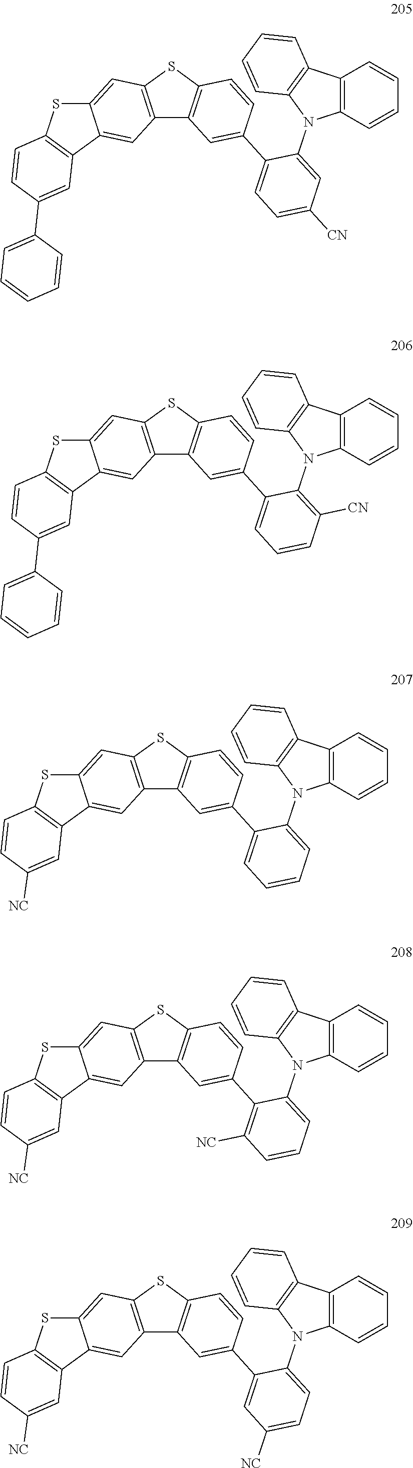 [SMILES: N#Cc1ccc(-c2ccc3sc4cc5sc6ccc(-c7ccccc7)cc6c5cc4c3c2)c(-n2c3ccccc3c3ccccc32)c1 ; N#Cc1cccc(-c2ccc3sc4cc5sc6ccc(-c7ccccc7)cc6c5cc4c3c2)c1-n1c2ccccc2c2ccccc21 ; N#Cc1ccc2sc3cc4sc5ccc(-c6ccccc6-n6c7ccccc7c7ccccc76)cc5c4cc3c2c1 ; N#Cc1ccc2sc3cc4sc5ccc(-c6c(C#N)cccc6-n6c7ccccc7c7ccccc76)cc5c4cc3c2c1 ; N#Cc1ccc(-n2c3ccccc3c3ccccc32)c(-c2ccc3sc4cc5sc6ccc(C#N)cc6c5cc4c3c2)c1]

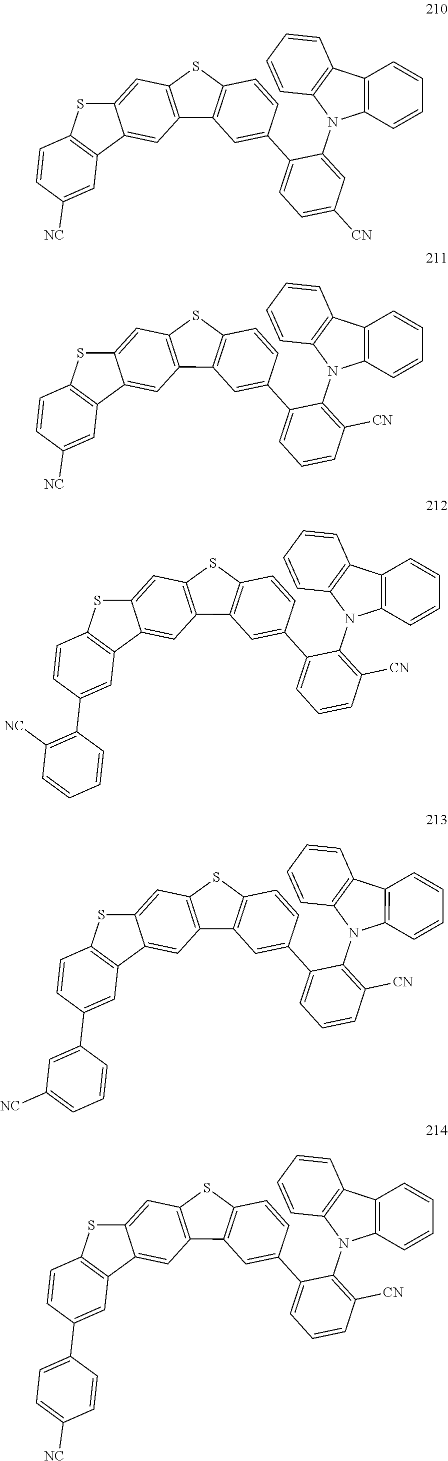 [SMILES: N#Cc1ccc(-c2ccc3sc4cc5sc6ccc(C#N)cc6c5cc4c3c2)c(-n2c3ccccc3c3ccccc32)c1 ; N#Cc1ccc2sc3cc4sc5ccc(-c6cccc(C#N)c6-n6c7ccccc7c7ccccc76)cc5c4cc3c2c1 ; N#Cc1ccccc1-c1ccc2sc3cc4sc5ccc(-c6cccc(C#N)c6-n6c7ccccc7c7ccccc76)cc5c4cc3c2c1 ; N#Cc1cccc(-c2ccc3sc4cc5sc6ccc(-c7cccc(C#N)c7-n7c8ccccc8c8ccccc87)cc6c5cc4c3c2)c1 ; N#Cc1ccc(-c2ccc3sc4cc5sc6ccc(-c7cccc(C#N)c7-n7c8ccccc8c8ccccc87)cc6c5cc4c3c2)cc1]

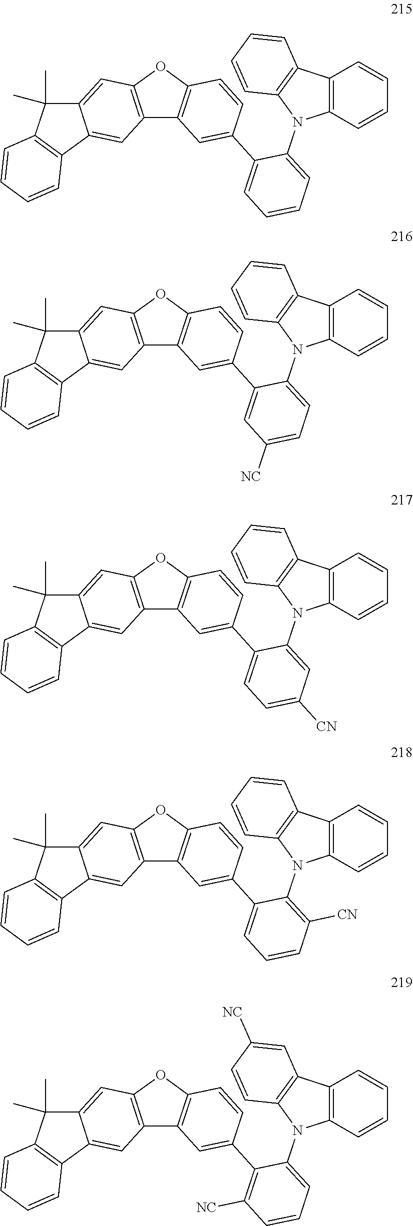 [SMILES: CC1(C)c2ccccc2-c2cc3c(cc21)oc1ccc(-c2ccccc2-n2c4ccccc4c4ccccc42)cc13 ; CC1(C)c2ccccc2-c2cc3c(cc21)oc1ccc(-c2cc(C#N)ccc2-n2c4ccccc4c4ccccc42)cc13 ; CC1(C)c2ccccc2-c2cc3c(cc21)oc1ccc(-c2ccc(C#N)cc2-n2c4ccccc4c4ccccc42)cc13 ; CC1(C)c2ccccc2-c2cc3c(cc21)oc1ccc(-c2cccc(C#N)c2-n2c4ccccc4c4ccccc42)cc13 ; CC1(C)c2ccccc2-c2cc3c(cc21)oc1ccc(-c2c(C#N)cccc2-n2c4ccccc4c4cc(C#N)ccc42)cc13]

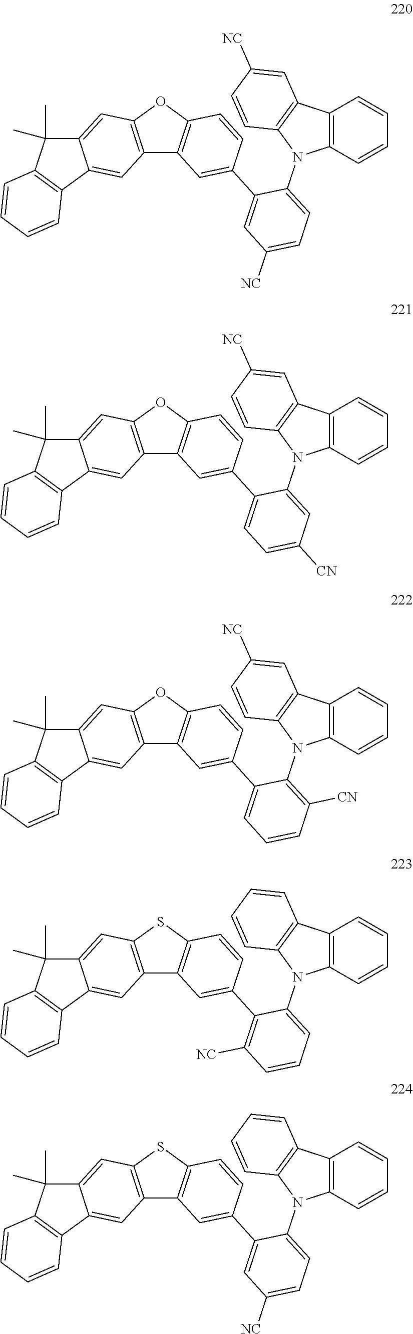

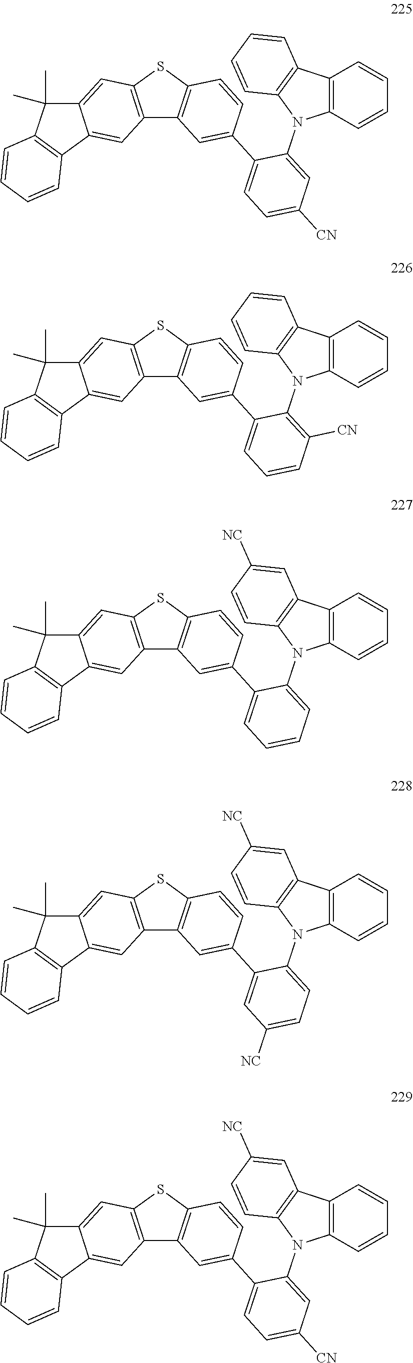 [SMILES: CC1(C)c2ccccc2-c2cc3c(cc21)sc1ccc(-c2ccc(C#N)cc2-n2c4ccccc4c4ccccc42)cc13 ; CC1(C)c2ccccc2-c2cc3c(cc21)sc1ccc(-c2cccc(C#N)c2-n2c4ccccc4c4ccccc42)cc13 ; CC1(C)c2ccccc2-c2cc3c(cc21)sc1ccc(-c2ccccc2-n2c4ccccc4c4cc(C#N)ccc42)cc13 ; CC1(C)c2ccccc2-c2cc3c(cc21)sc1ccc(-c2cc(C#N)ccc2-n2c4ccccc4c4cc(C#N)ccc42)cc13 ; CC1(C)c2ccccc2-c2cc3c(cc21)sc1ccc(-c2ccc(C#N)cc2-n2c4ccccc4c4cc(C#N)ccc42)cc13]

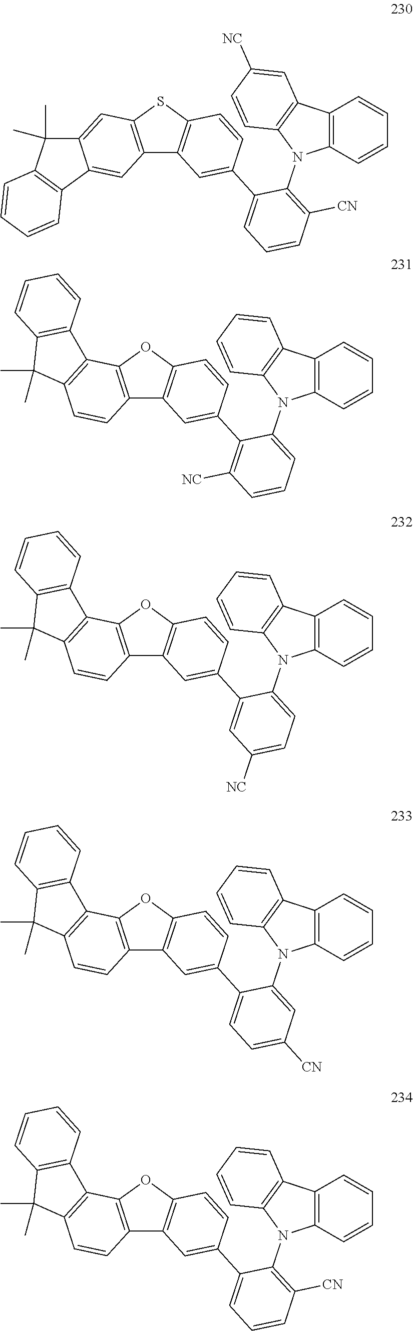

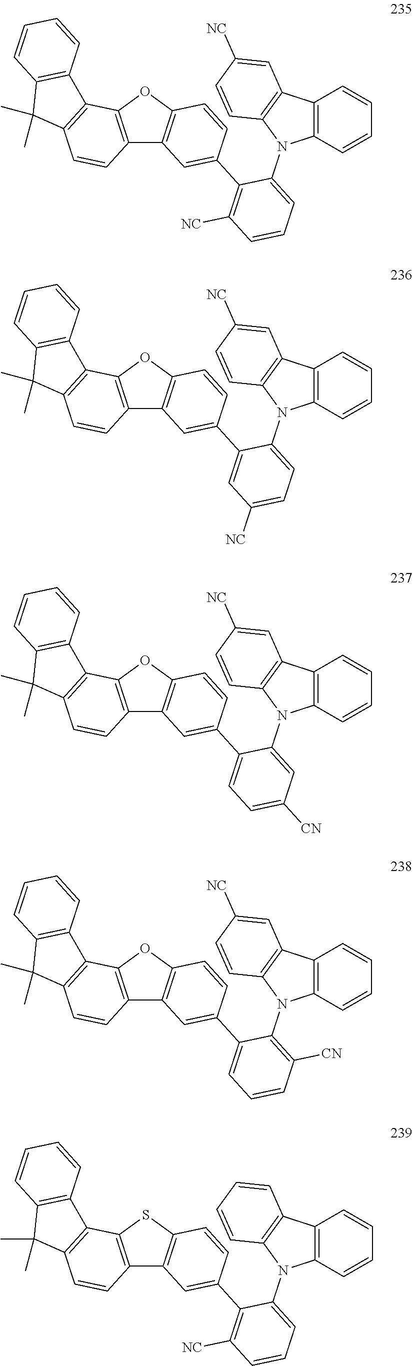 [SMILES: CC1(C)c2ccccc2-c2c1ccc1c2oc2ccc(-c3c(C#N)cccc3-n3c4ccccc4c4cc(C#N)ccc43)cc21 ; CC1(C)c2ccccc2-c2c1ccc1c2oc2ccc(-c3cc(C#N)ccc3-n3c4ccccc4c4cc(C#N)ccc43)cc21 ; CC1(C)c2ccccc2-c2c1ccc1c2oc2ccc(-c3ccc(C#N)cc3-n3c4ccccc4c4cc(C#N)ccc43)cc21 ; CC1(C)c2ccccc2-c2c1ccc1c2oc2ccc(-c3cccc(C#N)c3-n3c4ccccc4c4cc(C#N)ccc43)cc21 ; CC1(C)c2ccccc2-c2c1ccc1c2sc2ccc(-c3c(C#N)cccc3-n3c4ccccc4c4ccccc43)cc21]

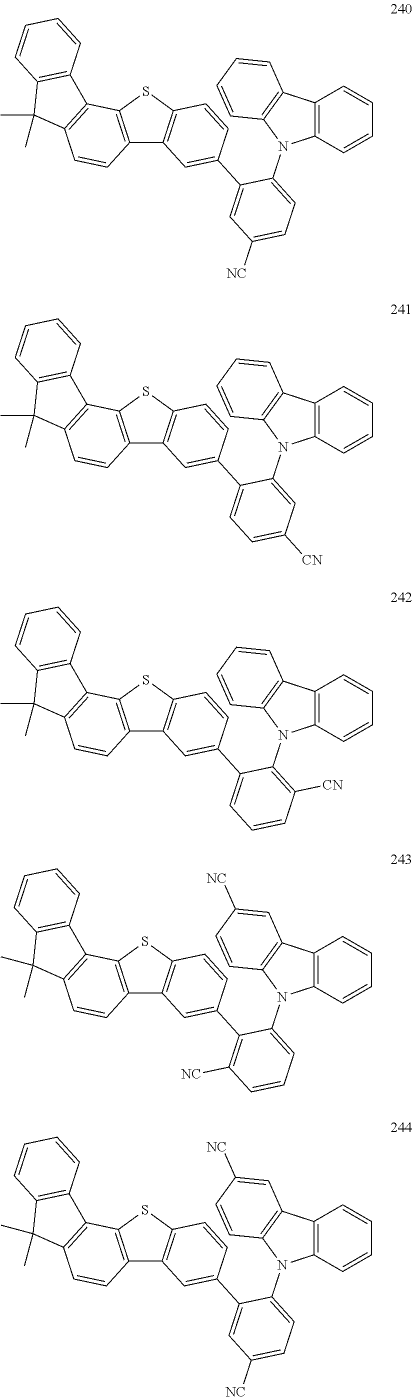 [SMILES: CC1(C)c2ccccc2-c2c1ccc1c2sc2ccc(-c3cc(C#N)ccc3-n3c4ccccc4c4ccccc43)cc21 ; CC1(C)c2ccccc2-c2c1ccc1c2sc2ccc(-c3ccc(C#N)cc3-n3c4ccccc4c4ccccc43)cc21 ; CC1(C)c2ccccc2-c2c1ccc1c2sc2ccc(-c3cccc(C#N)c3-n3c4ccccc4c4ccccc43)cc21 ; CC1(C)c2ccccc2-c2c1ccc1c2sc2ccc(-c3c(C#N)cccc3-n3c4ccccc4c4cc(C#N)ccc43)cc21 ; CC1(C)c2ccccc2-c2c1ccc1c2sc2ccc(-c3cc(C#N)ccc3-n3c4ccccc4c4cc(C#N)ccc43)cc21]

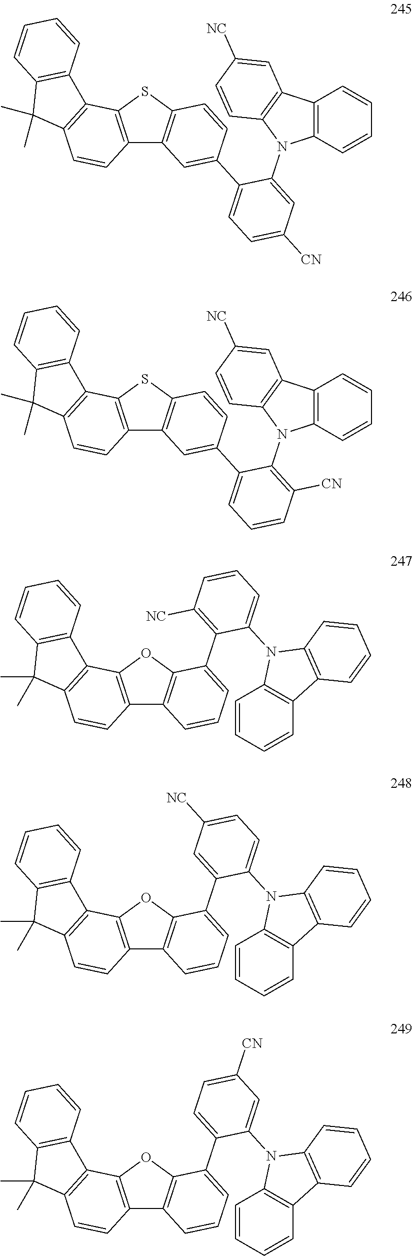

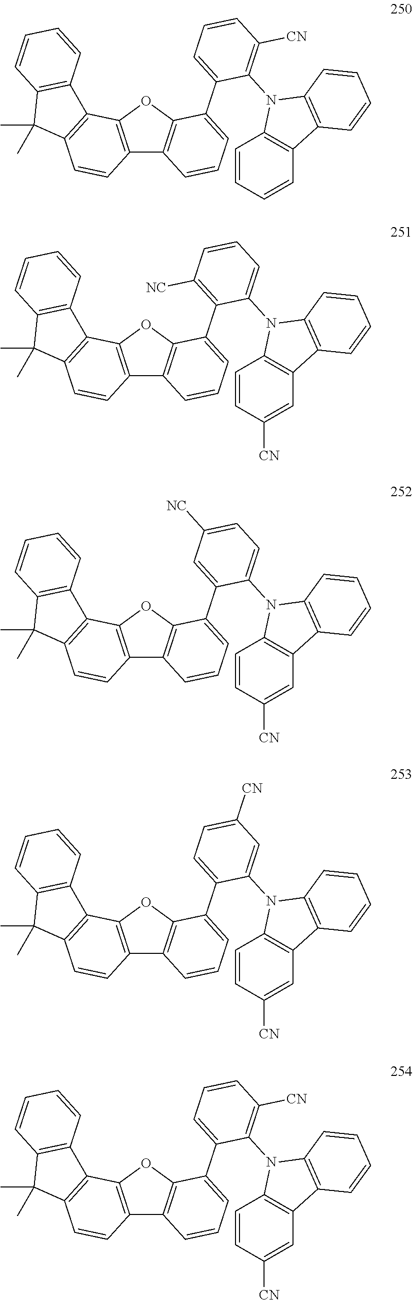 [SMILES: CC1(C)c2ccccc2-c2c1ccc1c2oc2c(-c3cccc(C#N)c3-n3c4ccccc4c4ccccc43)cccc21 ; CC1(C)c2ccccc2-c2c1ccc1c2oc2c(-c3c(C#N)cccc3-n3c4ccccc4c4cc(C#N)ccc43)cccc21 ; CC1(C)c2ccccc2-c2c1ccc1c2oc2c(-c3cc(C#N)ccc3-n3c4ccccc4c4cc(C#N)ccc43)cccc21 ; CC1(C)c2ccccc2-c2c1ccc1c2oc2c(-c3ccc(C#N)cc3-n3c4ccccc4c4cc(C#N)ccc43)cccc21 ; CC1(C)c2ccccc2-c2c1ccc1c2oc2c(-c3cccc(C#N)c3-n3c4ccccc4c4cc(C#N)ccc43)cccc21]

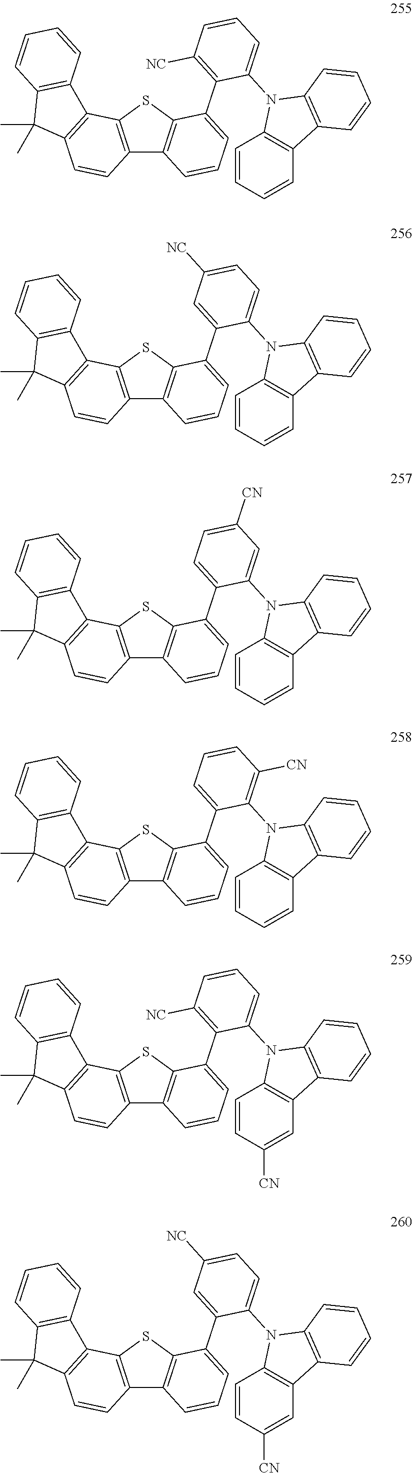 [SMILES: CC1(C)c2ccccc2-c2c1ccc1c2sc2c(-c3c(C#N)cccc3-n3c4ccccc4c4ccccc43)cccc21 ; CC1(C)c2ccccc2-c2c1ccc1c2sc2c(-c3cc(C#N)ccc3-n3c4ccccc4c4ccccc43)cccc21 ; CC1(C)c2ccccc2-c2c1ccc1c2sc2c(-c3ccc(C#N)cc3-n3c4ccccc4c4ccccc43)cccc21 ; CC1(C)c2ccccc2-c2c1ccc1c2sc2c(-c3cccc(C#N)c3-n3c4ccccc4c4ccccc43)cccc21 ; CC1(C)c2ccccc2-c2c1ccc1c2sc2c(-c3c(C#N)cccc3-n3c4ccccc4c4cc(C#N)ccc43)cccc21 ; CC1(C)c2ccccc2-c2c1ccc1c2sc2c(-c3cc(C#N)ccc3-n3c4ccccc4c4cc(C#N)ccc43)cccc21]

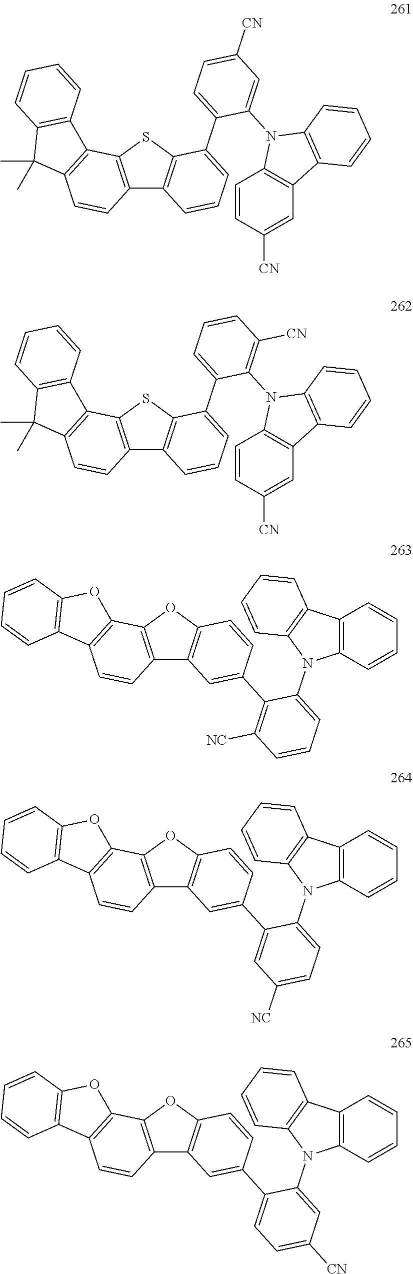

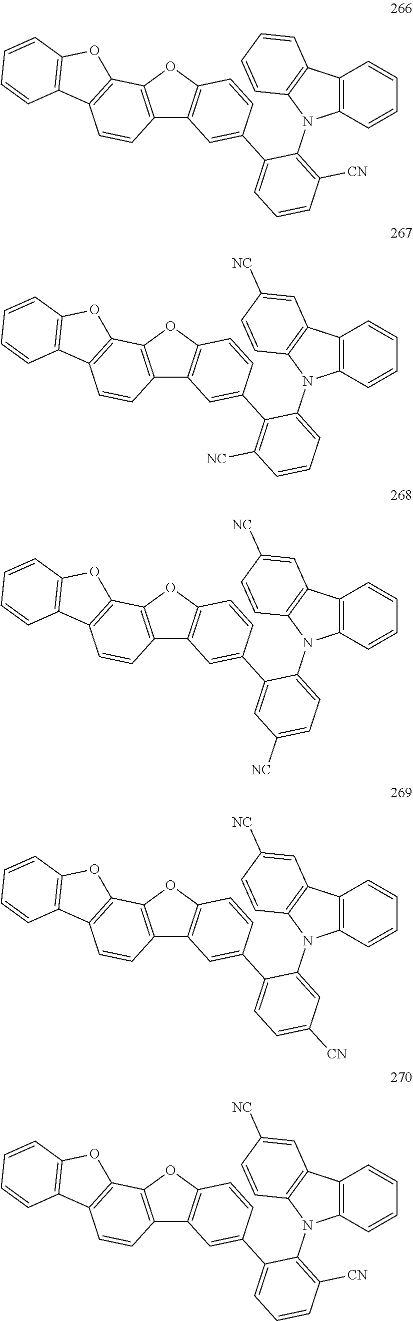 [SMILES: N#Cc1cccc(-c2ccc3oc4c(ccc5c6ccccc6oc54)c3c2)c1-n1c2ccccc2c2ccccc21 ; N#Cc1ccc2c(c1)c1ccccc1n2-c1cccc(C#N)c1-c1ccc2oc3c(ccc4c5ccccc5oc43)c2c1 ; N#Cc1ccc(-n2c3ccccc3c3cc(C#N)ccc32)c(-c2ccc3oc4c(ccc5c6ccccc6oc54)c3c2)c1 ; N#Cc1ccc(-c2ccc3oc4c(ccc5c6ccccc6oc54)c3c2)c(-n2c3ccccc3c3cc(C#N)ccc32)c1 ; N#Cc1ccc2c(c1)c1ccccc1n2-c1c(C#N)cccc1-c1ccc2oc3c(ccc4c5ccccc5oc43)c2c1]

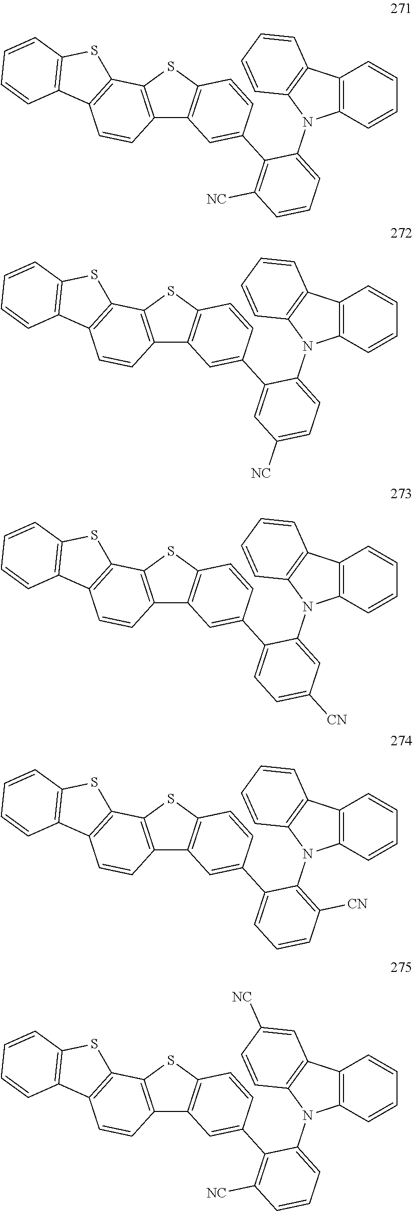

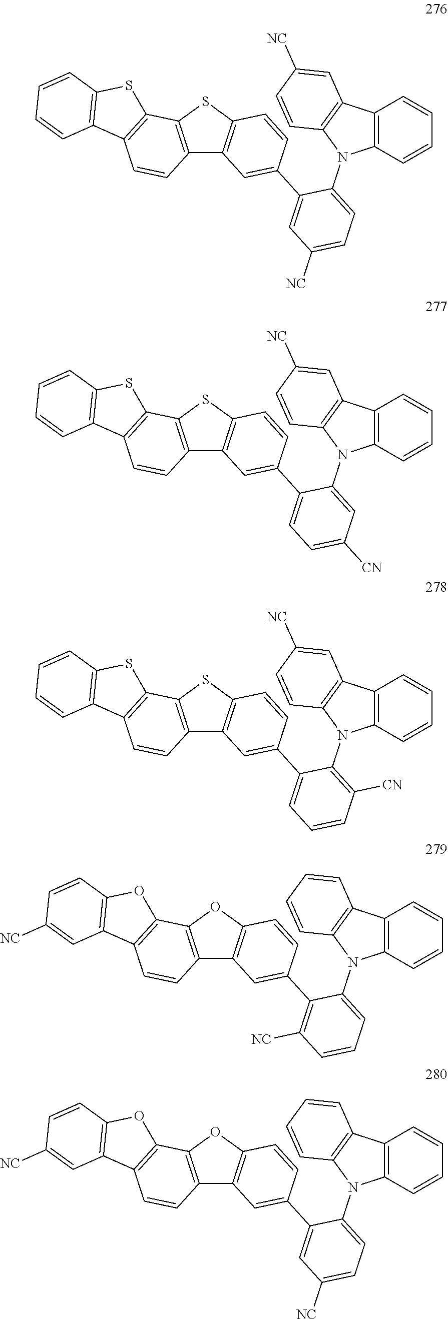

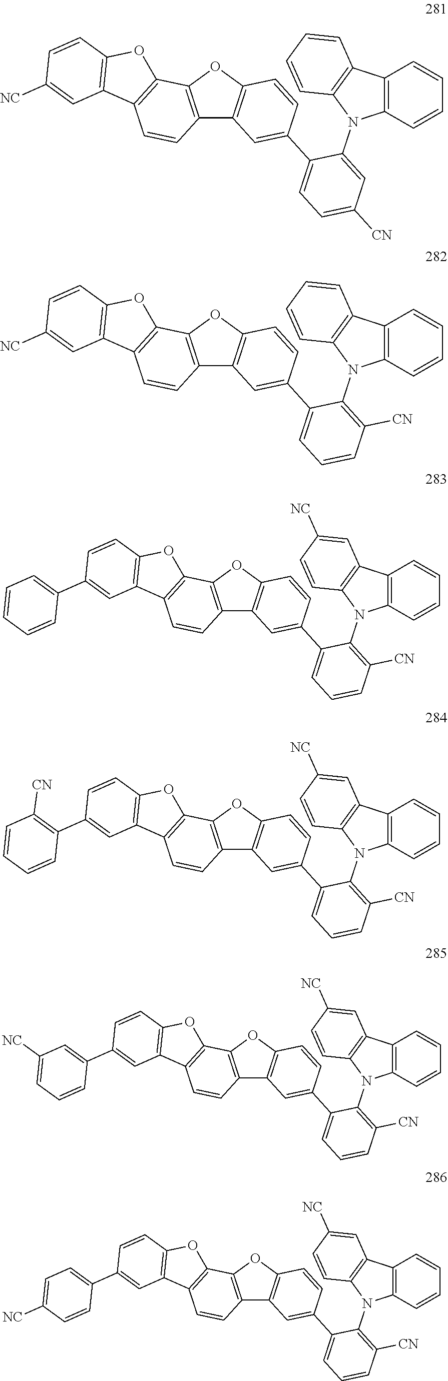 [SMILES: N#Cc1ccc(-c2ccc3oc4c(ccc5c6cc(C#N)ccc6oc54)c3c2)c(-n2c3ccccc3c3ccccc32)c1 ; N#Cc1ccc2oc3c(ccc4c5cc(-c6cccc(C#N)c6-n6c7ccccc7c7ccccc76)ccc5oc43)c2c1 ; N#Cc1ccc2c(c1)c1ccccc1n2-c1c(C#N)cccc1-c1ccc2oc3c(ccc4c5cc(-c6ccccc6)ccc5oc43)c2c1 ; N#Cc1ccc2c(c1)c1ccccc1n2-c1c(C#N)cccc1-c1ccc2oc3c(ccc4c5cc(-c6ccccc6C#N)ccc5oc43)c2c1 ; N#Cc1cccc(-c2ccc3oc4c(ccc5c6cc(-c7cccc(C#N)c7-n7c8ccccc8c8cc(C#N)ccc87)ccc6oc54)c3c2)c1 ; N#Cc1ccc(-c2ccc3oc4c(ccc5c6cc(-c7cccc(C#N)c7-n7c8ccccc8c8cc(C#N)ccc87)ccc6oc54)c3c2)cc1]

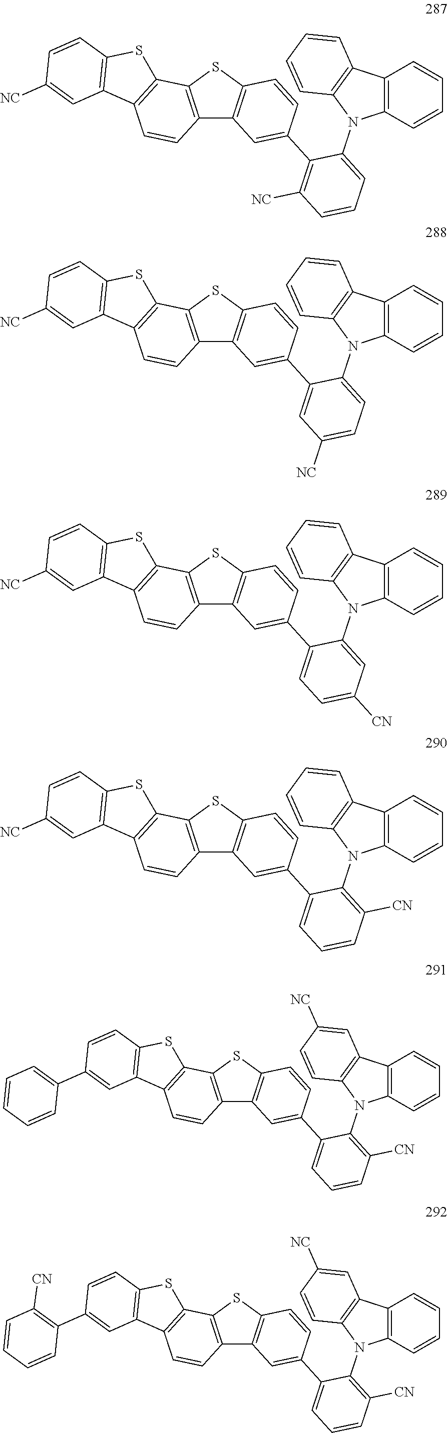

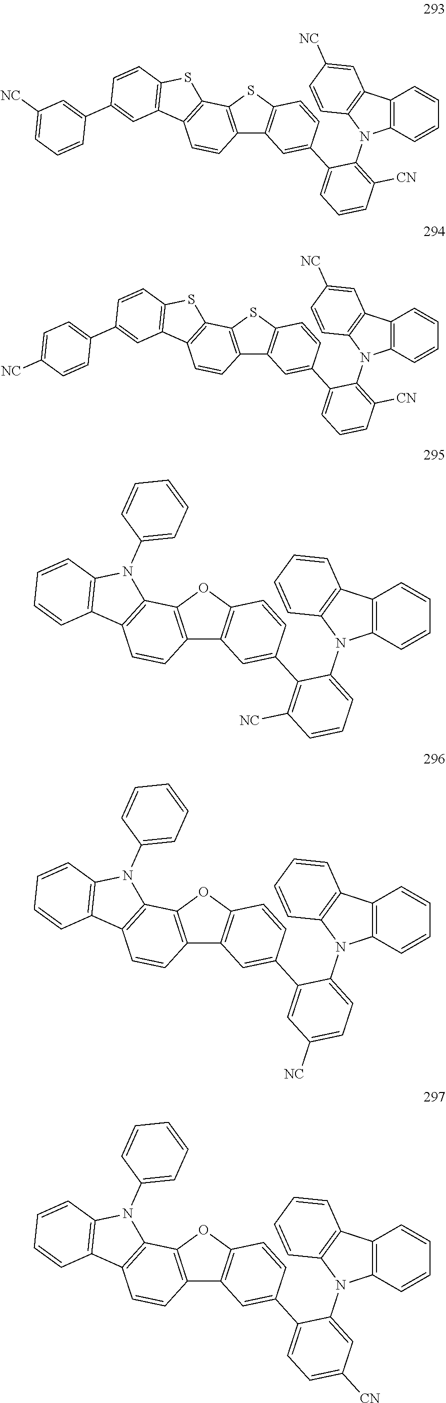 [SMILES: N#Cc1cccc(-c2ccc3sc4c(ccc5c6cc(-c7cccc(C#N)c7-n7c8ccccc8c8cc(C#N)ccc87)ccc6sc54)c3c2)c1 ; N#Cc1ccc(-c2ccc3sc4c(ccc5c6cc(-c7cccc(C#N)c7-n7c8ccccc8c8cc(C#N)ccc87)ccc6sc54)c3c2)cc1 ; N#Cc1cccc(-n2c3ccccc3c3ccccc32)c1-c1ccc2oc3c(ccc4c5ccccc5n(-c5ccccc5)c43)c2c1 ; N#Cc1ccc(-n2c3ccccc3c3ccccc32)c(-c2ccc3oc4c(ccc5c6ccccc6n(-c6ccccc6)c54)c3c2)c1 ; N#Cc1ccc(-c2ccc3oc4c(ccc5c6ccccc6n(-c6ccccc6)c54)c3c2)c(-n2c3ccccc3c3ccccc32)c1]

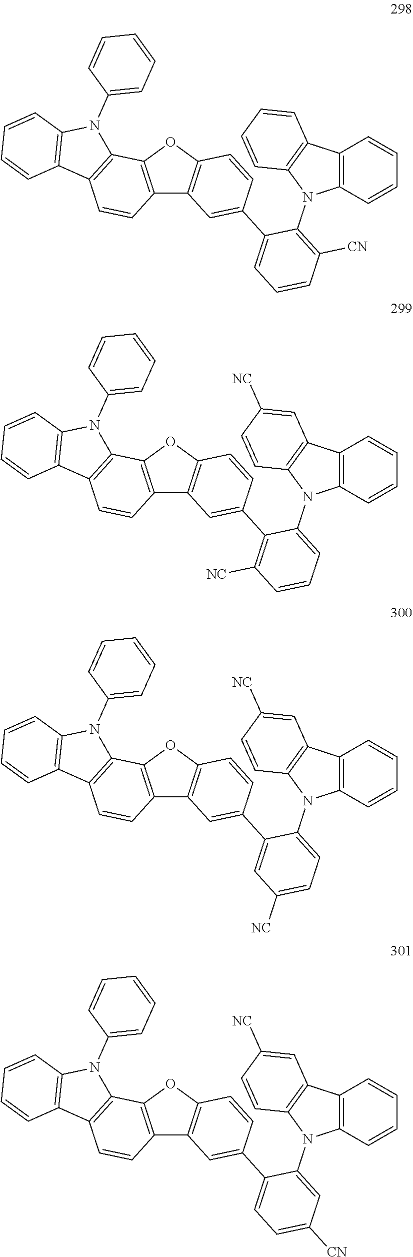 [SMILES: N#Cc1cccc(-c2ccc3oc4c(ccc5c6ccccc6n(-c6ccccc6)c54)c3c2)c1-n1c2ccccc2c2ccccc21 ; N#Cc1ccc2c(c1)c1ccccc1n2-c1cccc(C#N)c1-c1ccc2oc3c(ccc4c5ccccc5n(-c5ccccc5)c43)c2c1 ; N#Cc1ccc(-n2c3ccccc3c3cc(C#N)ccc32)c(-c2ccc3oc4c(ccc5c6ccccc6n(-c6ccccc6)c54)c3c2)c1 ; N#Cc1ccc(-c2ccc3oc4c(ccc5c6ccccc6n(-c6ccccc6)c54)c3c2)c(-n2c3ccccc3c3cc(C#N)ccc32)c1]

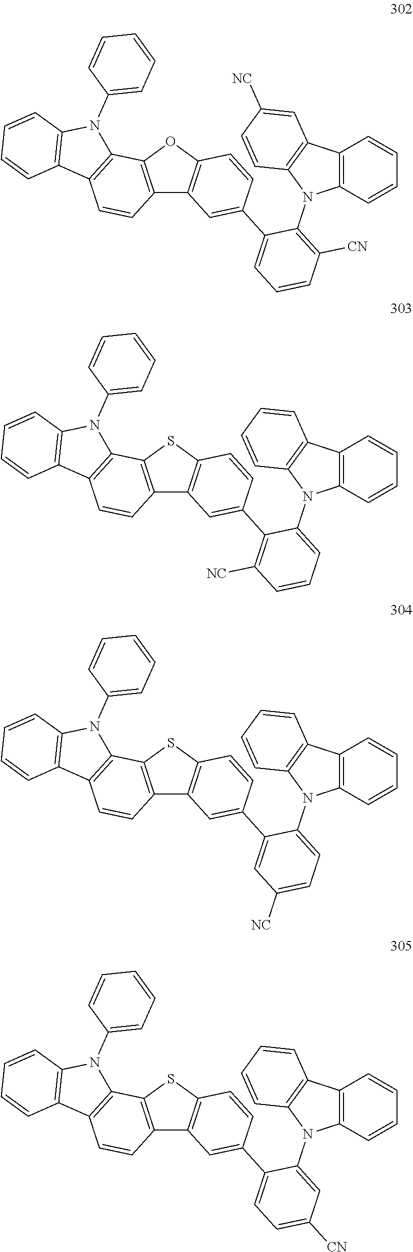 [SMILES: N#Cc1ccc2c(c1)c1ccccc1n2-c1c(C#N)cccc1-c1ccc2oc3c(ccc4c5ccccc5n(-c5ccccc5)c43)c2c1 ; N#Cc1cccc(-n2c3ccccc3c3ccccc32)c1-c1ccc2sc3c(ccc4c5ccccc5n(-c5ccccc5)c43)c2c1 ; N#Cc1ccc(-n2c3ccccc3c3ccccc32)c(-c2ccc3sc4c(ccc5c6ccccc6n(-c6ccccc6)c54)c3c2)c1 ; N#Cc1ccc(-c2ccc3sc4c(ccc5c6ccccc6n(-c6ccccc6)c54)c3c2)c(-n2c3ccccc3c3ccccc32)c1]

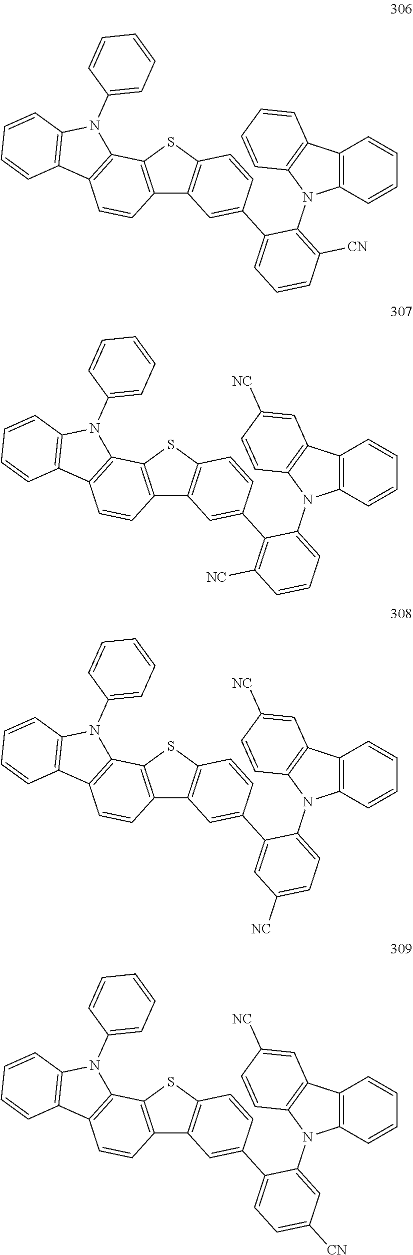 [SMILES: N#Cc1cccc(-c2ccc3sc4c(ccc5c6ccccc6n(-c6ccccc6)c54)c3c2)c1-n1c2ccccc2c2ccccc21 ; N#Cc1ccc2c(c1)c1ccccc1n2-c1cccc(C#N)c1-c1ccc2sc3c(ccc4c5ccccc5n(-c5ccccc5)c43)c2c1 ; N#Cc1ccc(-n2c3ccccc3c3cc(C#N)ccc32)c(-c2ccc3sc4c(ccc5c6ccccc6n(-c6ccccc6)c54)c3c2)c1 ; N#Cc1ccc(-c2ccc3sc4c(ccc5c6ccccc6n(-c6ccccc6)c54)c3c2)c(-n2c3ccccc3c3cc(C#N)ccc32)c1]

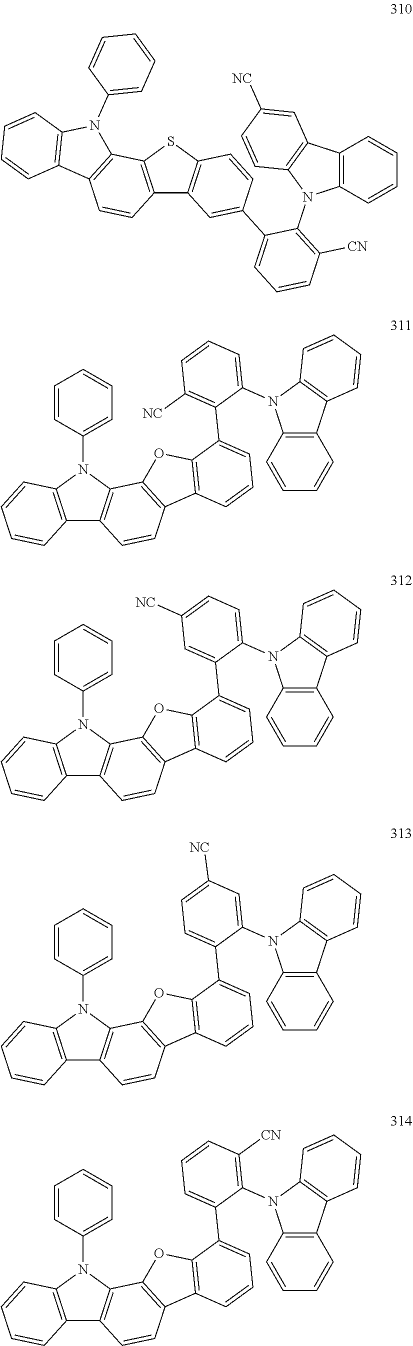 [SMILES: N#Cc1ccc2c(c1)c1ccccc1n2-c1c(C#N)cccc1-c1ccc2sc3c(ccc4c5ccccc5n(-c5ccccc5)c43)c2c1 ; N#Cc1cccc(-n2c3ccccc3c3ccccc32)c1-c1cccc2c1oc1c2ccc2c3ccccc3n(-c3ccccc3)c21 ; N#Cc1ccc(-n2c3ccccc3c3ccccc32)c(-c2cccc3c2oc2c3ccc3c4ccccc4n(-c4ccccc4)c32)c1 ; N#Cc1ccc(-c2cccc3c2oc2c3ccc3c4ccccc4n(-c4ccccc4)c32)c(-n2c3ccccc3c3ccccc32)c1 ; N#Cc1cccc(-c2cccc3c2oc2c3ccc3c4ccccc4n(-c4ccccc4)c32)c1-n1c2ccccc2c2ccccc21]

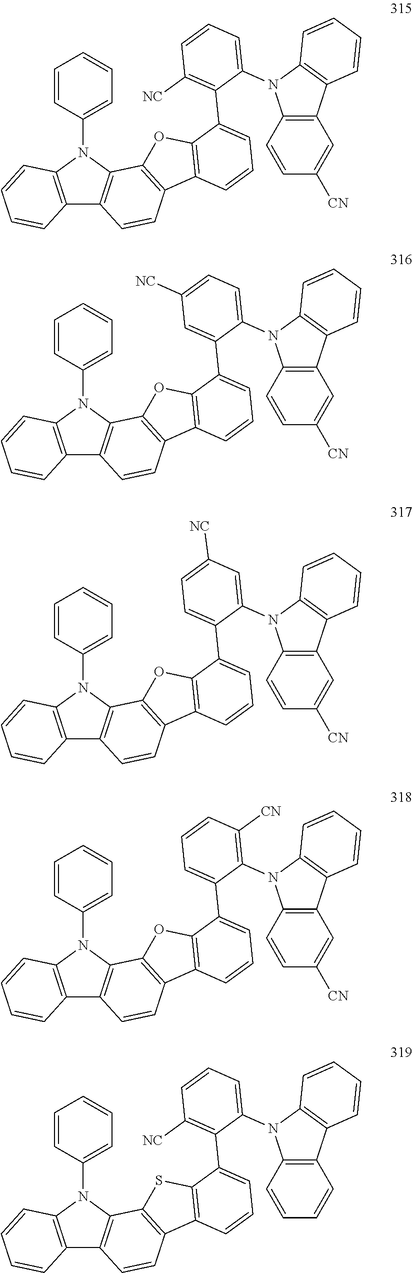 [SMILES: N#Cc1ccc2c(c1)c1ccccc1n2-c1cccc(C#N)c1-c1cccc2c1oc1c2ccc2c3ccccc3n(-c3ccccc3)c21 ; N#Cc1ccc(-n2c3ccccc3c3cc(C#N)ccc32)c(-c2cccc3c2oc2c3ccc3c4ccccc4n(-c4ccccc4)c32)c1 ; N#Cc1ccc(-c2cccc3c2oc2c3ccc3c4ccccc4n(-c4ccccc4)c32)c(-n2c3ccccc3c3cc(C#N)ccc32)c1 ; N#Cc1ccc2c(c1)c1ccccc1n2-c1c(C#N)cccc1-c1cccc2c1oc1c2ccc2c3ccccc3n(-c3ccccc3)c21 ; N#Cc1cccc(-n2c3ccccc3c3ccccc32)c1-c1cccc2c1sc1c2ccc2c3ccccc3n(-c3ccccc3)c21]

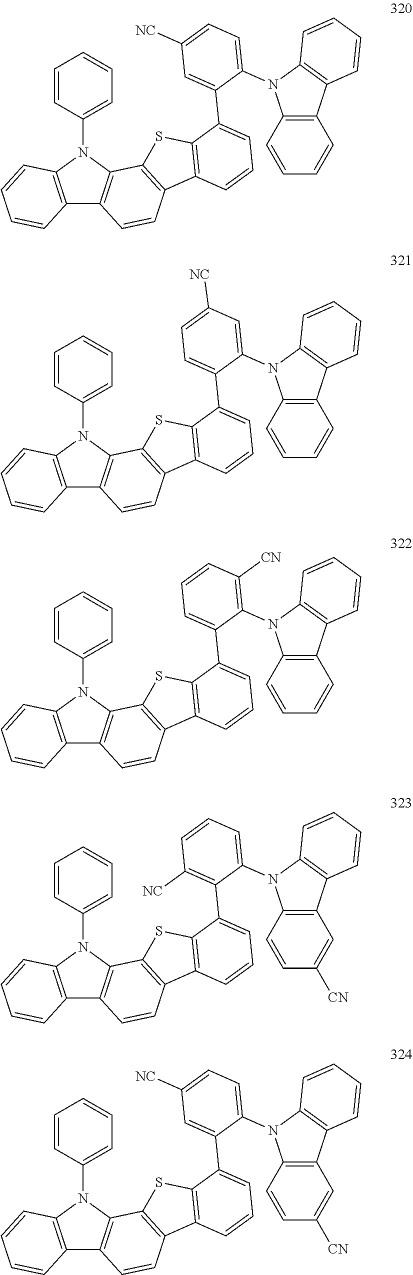 [SMILES: N#Cc1ccc(-n2c3ccccc3c3ccccc32)c(-c2cccc3c2sc2c3ccc3c4ccccc4n(-c4ccccc4)c32)c1 ; N#Cc1ccc(-c2cccc3c2sc2c3ccc3c4ccccc4n(-c4ccccc4)c32)c(-n2c3ccccc3c3ccccc32)c1 ; N#Cc1cccc(-c2cccc3c2sc2c3ccc3c4ccccc4n(-c4ccccc4)c32)c1-n1c2ccccc2c2ccccc21 ; N#Cc1ccc2c(c1)c1ccccc1n2-c1cccc(C#N)c1-c1cccc2c1sc1c2ccc2c3ccccc3n(-c3ccccc3)c21 ; N#Cc1ccc(-n2c3ccccc3c3cc(C#N)ccc32)c(-c2cccc3c2sc2c3ccc3c4ccccc4n(-c4ccccc4)c32)c1]

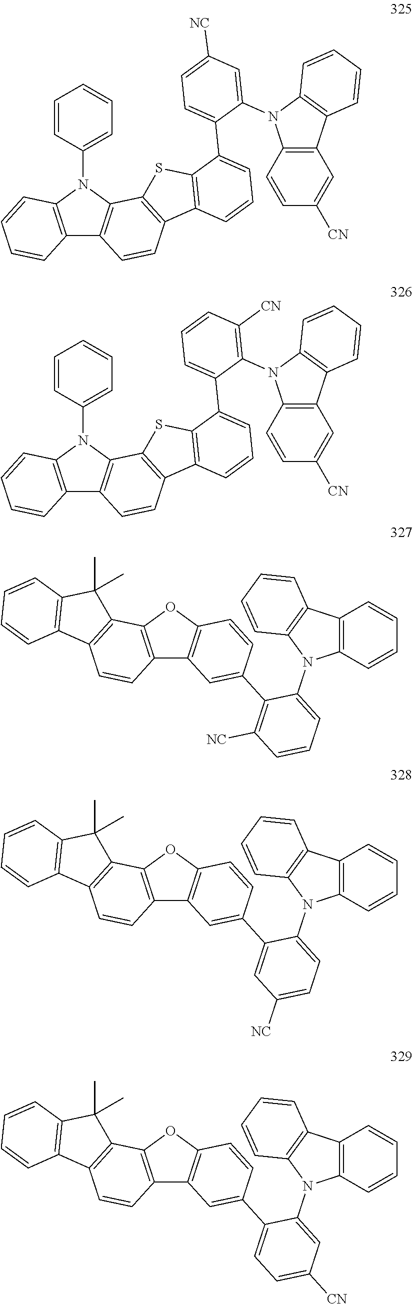 [SMILES: N#Cc1ccc(-c2cccc3c2sc2c3ccc3c4ccccc4n(-c4ccccc4)c32)c(-n2c3ccccc3c3cc(C#N)ccc32)c1 ; N#Cc1ccc2c(c1)c1ccccc1n2-c1c(C#N)cccc1-c1cccc2c1sc1c2ccc2c3ccccc3n(-c3ccccc3)c21 ; CC1(C)c2ccccc2-c2ccc3c(oc4ccc(-c5c(C#N)cccc5-n5c6ccccc6c6ccccc65)cc43)c21 ; CC1(C)c2ccccc2-c2ccc3c(oc4ccc(-c5cc(C#N)ccc5-n5c6ccccc6c6ccccc65)cc43)c21 ; CC1(C)c2ccccc2-c2ccc3c(oc4ccc(-c5ccc(C#N)cc5-n5c6ccccc6c6ccccc65)cc43)c21]

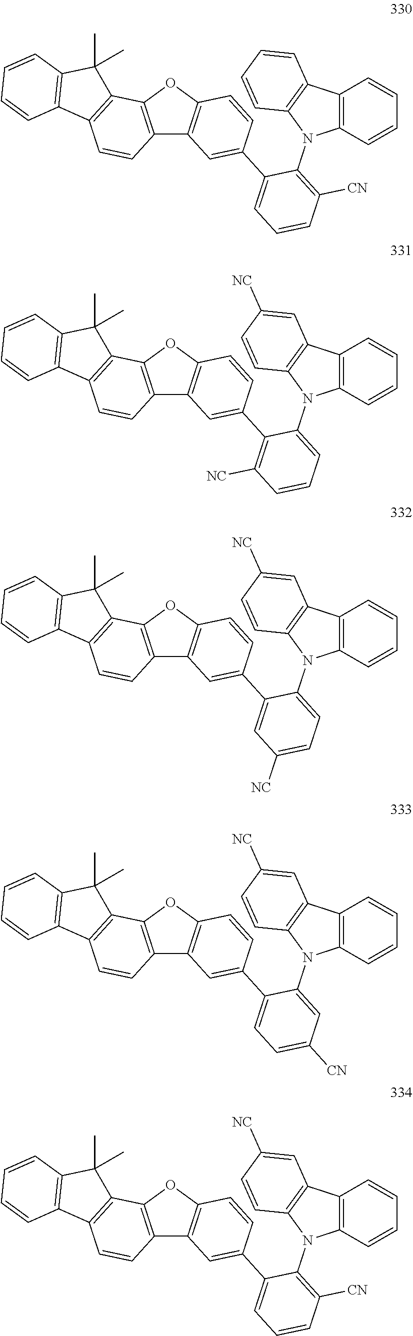 [SMILES: CC1(C)c2ccccc2-c2ccc3c(oc4ccc(-c5cccc(C#N)c5-n5c6ccccc6c6ccccc65)cc43)c21 ; CC1(C)c2ccccc2-c2ccc3c(oc4ccc(-c5c(C#N)cccc5-n5c6ccccc6c6cc(C#N)ccc65)cc43)c21 ; CC1(C)c2ccccc2-c2ccc3c(oc4ccc(-c5cc(C#N)ccc5-n5c6ccccc6c6cc(C#N)ccc65)cc43)c21 ; CC1(C)c2ccccc2-c2ccc3c(oc4ccc(-c5ccc(C#N)cc5-n5c6ccccc6c6cc(C#N)ccc65)cc43)c21 ; CC1(C)c2ccccc2-c2ccc3c(oc4ccc(-c5cccc(C#N)c5-n5c6ccccc6c6cc(C#N)ccc65)cc43)c21]

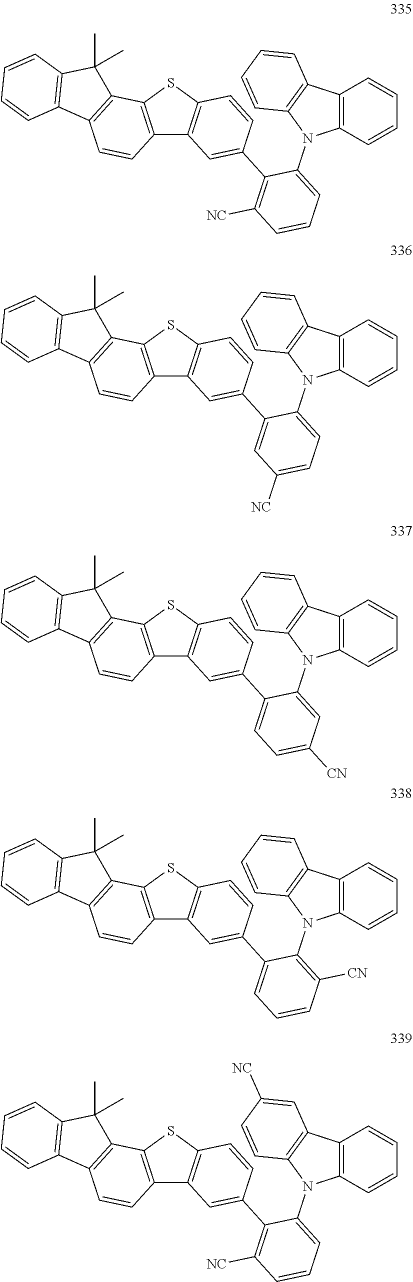 [SMILES: CC1(C)c2ccccc2-c2ccc3c(sc4ccc(-c5c(C#N)cccc5-n5c6ccccc6c6ccccc65)cc43)c21 ; CC1(C)c2ccccc2-c2ccc3c(sc4ccc(-c5cc(C#N)ccc5-n5c6ccccc6c6ccccc65)cc43)c21 ; CC1(C)c2ccccc2-c2ccc3c(sc4ccc(-c5ccc(C#N)cc5-n5c6ccccc6c6ccccc65)cc43)c21 ; CC1(C)c2ccccc2-c2ccc3c(sc4ccc(-c5cccc(C#N)c5-n5c6ccccc6c6ccccc65)cc43)c21 ; CC1(C)c2ccccc2-c2ccc3c(sc4ccc(-c5c(C#N)cccc5-n5c6ccccc6c6cc(C#N)ccc65)cc43)c21]

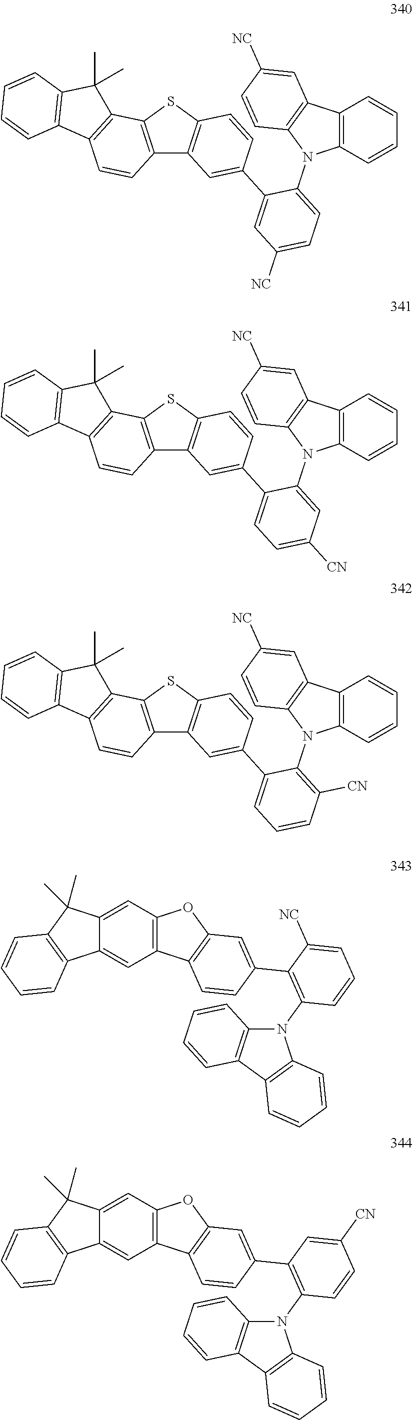 [SMILES: CC1(C)c2ccccc2-c2ccc3c(sc4ccc(-c5cc(C#N)ccc5-n5c6ccccc6c6cc(C#N)ccc65)cc43)c21 ; CC1(C)c2ccccc2-c2ccc3c(sc4ccc(-c5ccc(C#N)cc5-n5c6ccccc6c6cc(C#N)ccc65)cc43)c21 ; CC1(C)c2ccccc2-c2ccc3c(sc4ccc(-c5cccc(C#N)c5-n5c6ccccc6c6cc(C#N)ccc65)cc43)c21 ; CC1(C)c2ccccc2-c2cc3c(cc21)oc1cc(-c2c(C#N)cccc2-n2c4ccccc4c4ccccc42)ccc13 ; CC1(C)c2ccccc2-c2cc3c(cc21)oc1cc(-c2cc(C#N)ccc2-n2c4ccccc4c4ccccc42)ccc13]

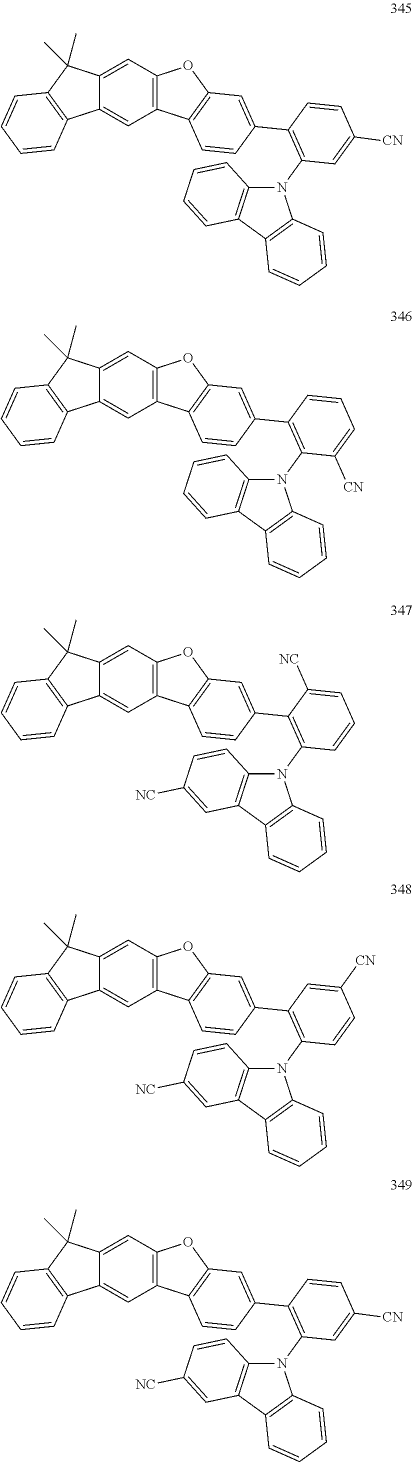 [SMILES: CC1(C)c2ccccc2-c2cc3c(cc21)oc1cc(-c2ccc(C#N)cc2-n2c4ccccc4c4ccccc42)ccc13 ; CC1(C)c2ccccc2-c2cc3c(cc21)oc1cc(-c2cccc(C#N)c2-n2c4ccccc4c4ccccc42)ccc13 ; CC1(C)c2ccccc2-c2cc3c(cc21)oc1cc(-c2c(C#N)cccc2-n2c4ccccc4c4cc(C#N)ccc42)ccc13 ; CC1(C)c2ccccc2-c2cc3c(cc21)oc1cc(-c2cc(C#N)ccc2-n2c4ccccc4c4cc(C#N)ccc42)ccc13 ; CC1(C)c2ccccc2-c2cc3c(cc21)oc1cc(-c2ccc(C#N)cc2-n2c4ccccc4c4cc(C#N)ccc42)ccc13]

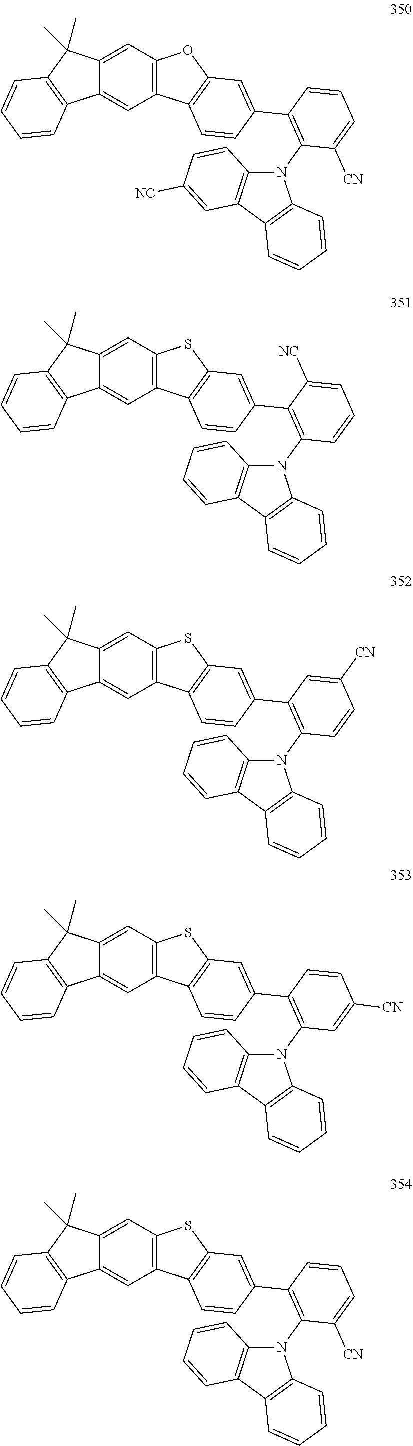 [SMILES: CC1(C)c2ccccc2-c2cc3c(cc21)oc1cc(-c2cccc(C#N)c2-n2c4ccccc4c4cc(C#N)ccc42)ccc13 ; CC1(C)c2ccccc2-c2cc3c(cc21)sc1cc(-c2c(C#N)cccc2-n2c4ccccc4c4ccccc42)ccc13 ; CC1(C)c2ccccc2-c2cc3c(cc21)sc1cc(-c2cc(C#N)ccc2-n2c4ccccc4c4ccccc42)ccc13 ; CC1(C)c2ccccc2-c2cc3c(cc21)sc1cc(-c2ccc(C#N)cc2-n2c4ccccc4c4ccccc42)ccc13 ; CC1(C)c2ccccc2-c2cc3c(cc21)sc1cc(-c2cccc(C#N)c2-n2c4ccccc4c4ccccc42)ccc13]

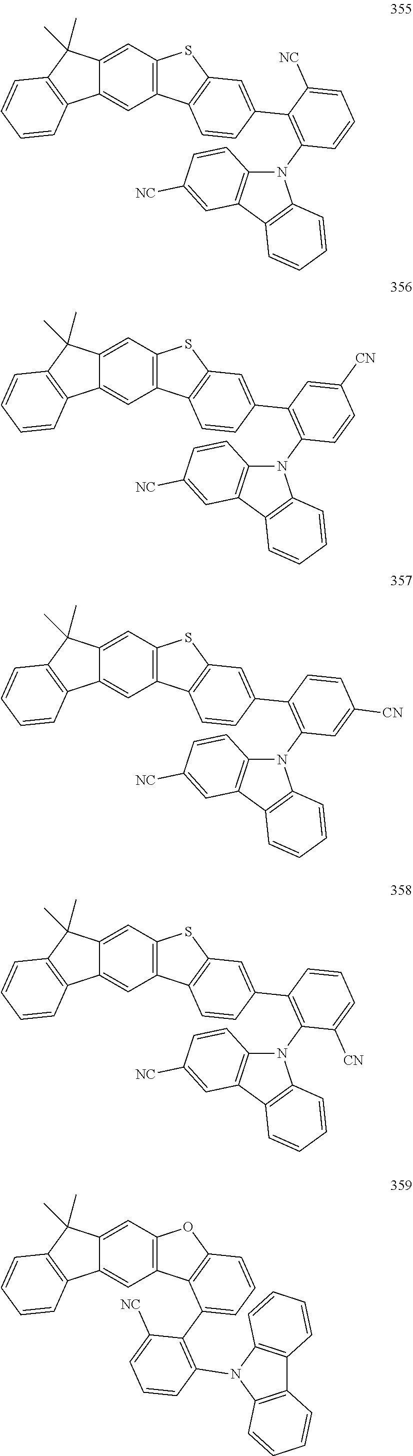 [SMILES: CC1(C)c2ccccc2-c2cc3c(cc21)sc1cc(-c2c(C#N)cccc2-n2c4ccccc4c4cc(C#N)ccc42)ccc13 ; CC1(C)c2ccccc2-c2cc3c(cc21)sc1cc(-c2cc(C#N)ccc2-n2c4ccccc4c4cc(C#N)ccc42)ccc13 ; CC1(C)c2ccccc2-c2cc3c(cc21)sc1cc(-c2ccc(C#N)cc2-n2c4ccccc4c4cc(C#N)ccc42)ccc13 ; CC1(C)c2ccccc2-c2cc3c(cc21)sc1cc(-c2cccc(C#N)c2-n2c4ccccc4c4cc(C#N)ccc42)ccc13 ; CC1(C)c2ccccc2-c2cc3c(cc21)oc1cccc(-c2c(C#N)cccc2-n2c4ccccc4c4ccccc42)c13]

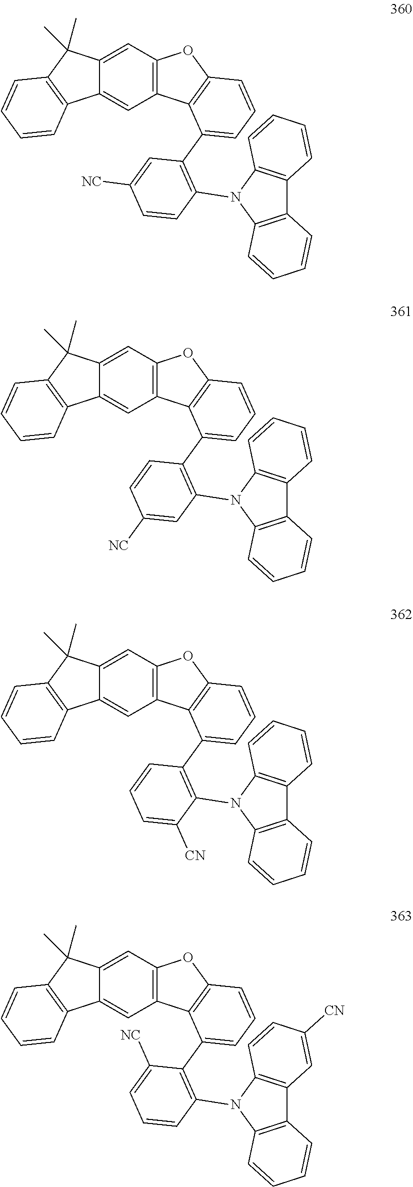 [SMILES: CC1(C)c2ccccc2-c2cc3c(cc21)oc1cccc(-c2cc(C#N)ccc2-n2c4ccccc4c4ccccc42)c13 ; CC1(C)c2ccccc2-c2cc3c(cc21)oc1cccc(-c2ccc(C#N)cc2-n2c4ccccc4c4ccccc42)c13 ; CC1(C)c2ccccc2-c2cc3c(cc21)oc1cccc(-c2cccc(C#N)c2-n2c4ccccc4c4ccccc42)c13 ; CC1(C)c2ccccc2-c2cc3c(cc21)oc1cccc(-c2c(C#N)cccc2-n2c4ccccc4c4cc(C#N)ccc42)c13]

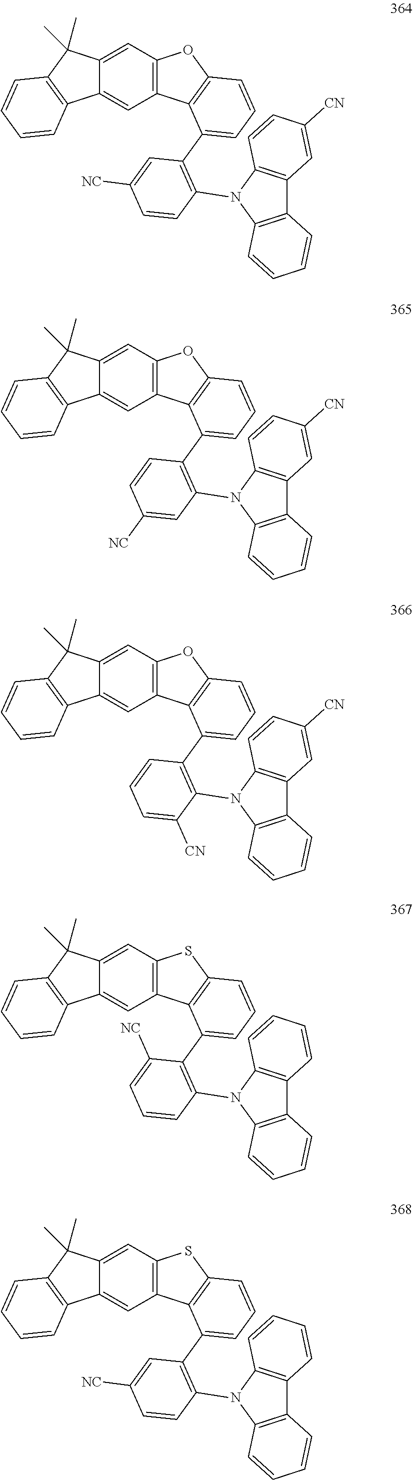 [SMILES: CC1(C)c2ccccc2-c2cc3c(cc21)oc1cccc(-c2cc(C#N)ccc2-n2c4ccccc4c4cc(C#N)ccc42)c13 ; CC1(C)c2ccccc2-c2cc3c(cc21)oc1cccc(-c2ccc(C#N)cc2-n2c4ccccc4c4cc(C#N)ccc42)c13 ; CC1(C)c2ccccc2-c2cc3c(cc21)oc1cccc(-c2cccc(C#N)c2-n2c4ccccc4c4cc(C#N)ccc42)c13 ; CC1(C)c2ccccc2-c2cc3c(cc21)sc1cccc(-c2c(C#N)cccc2-n2c4ccccc4c4ccccc42)c13 ; CC1(C)c2ccccc2-c2cc3c(cc21)sc1cccc(-c2cc(C#N)ccc2-n2c4ccccc4c4ccccc42)c13]

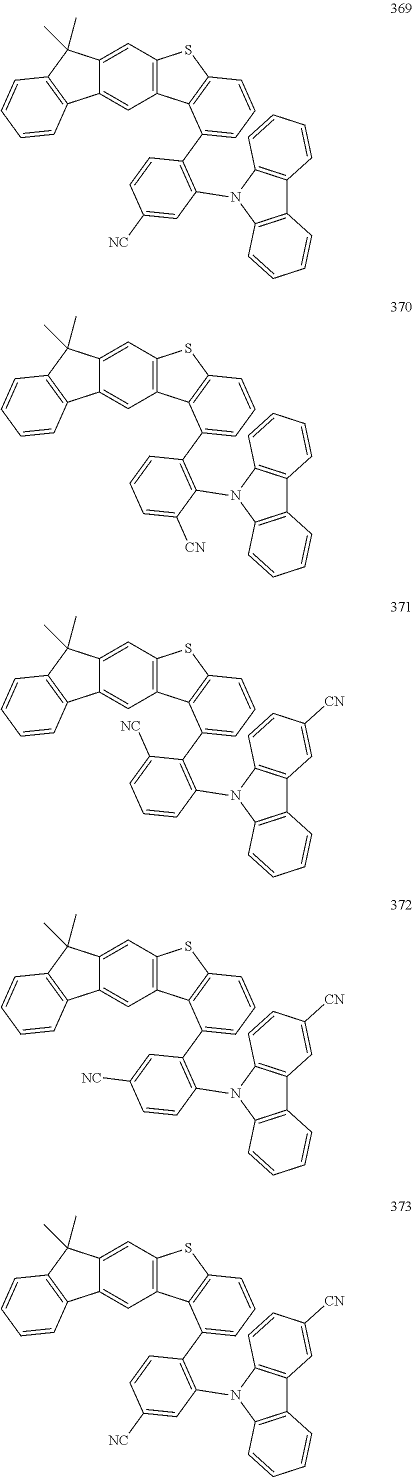 [SMILES: CC1(C)c2ccccc2-c2cc3c(cc21)sc1cccc(-c2ccc(C#N)cc2-n2c4ccccc4c4ccccc42)c13 ; CC1(C)c2ccccc2-c2cc3c(cc21)sc1cccc(-c2cccc(C#N)c2-n2c4ccccc4c4ccccc42)c13 ; CC1(C)c2ccccc2-c2cc3c(cc21)sc1cccc(-c2c(C#N)cccc2-n2c4ccccc4c4cc(C#N)ccc42)c13 ; CC1(C)c2ccccc2-c2cc3c(cc21)sc1cccc(-c2cc(C#N)ccc2-n2c4ccccc4c4cc(C#N)ccc42)c13 ; CC1(C)c2ccccc2-c2cc3c(cc21)sc1cccc(-c2ccc(C#N)cc2-n2c4ccccc4c4cc(C#N)ccc42)c13]

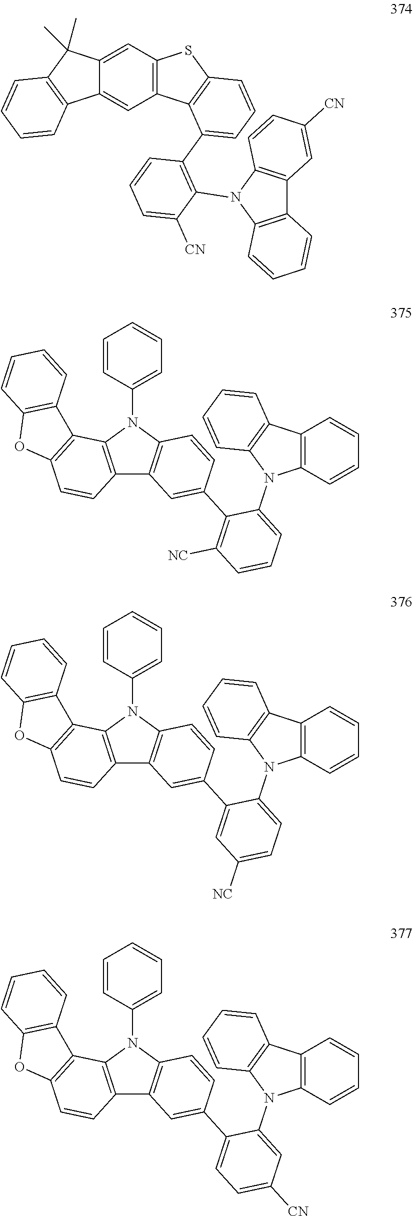 [SMILES: CC1(C)c2ccccc2-c2cc3c(cc21)sc1cccc(-c2cccc(C#N)c2-n2c4ccccc4c4cc(C#N)ccc42)c13 ; N#Cc1cccc(-n2c3ccccc3c3ccccc32)c1-c1ccc2c(c1)c1ccc3oc4ccccc4c3c1n2-c1ccccc1 ; N#Cc1ccc(-n2c3ccccc3c3ccccc32)c(-c2ccc3c(c2)c2ccc4oc5ccccc5c4c2n3-c2ccccc2)c1 ; N#Cc1ccc(-c2ccc3c(c2)c2ccc4oc5ccccc5c4c2n3-c2ccccc2)c(-n2c3ccccc3c3ccccc32)c1]

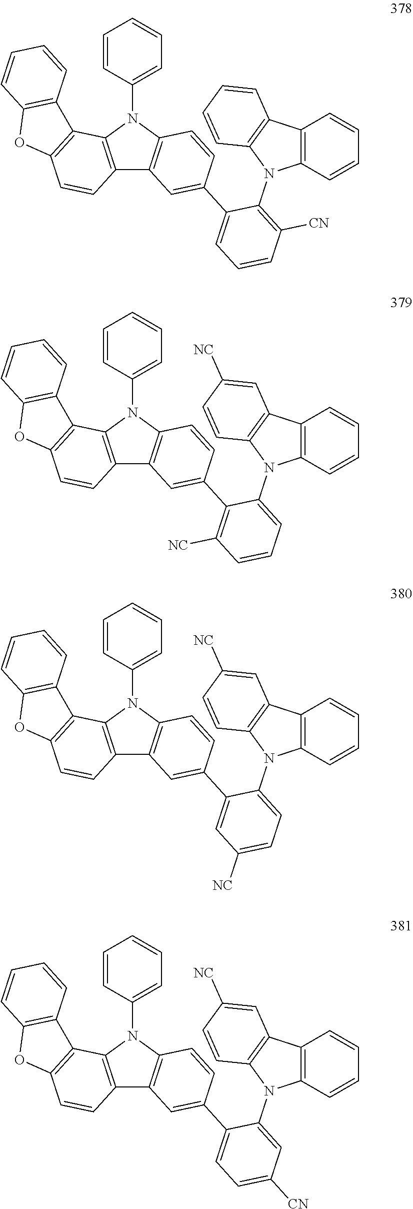 [SMILES: N#Cc1cccc(-c2ccc3c(c2)c2ccc4oc5ccccc5c4c2n3-c2ccccc2)c1-n1c2ccccc2c2ccccc21 ; N#Cc1ccc2c(c1)c1ccccc1n2-c1cccc(C#N)c1-c1ccc2c(c1)c1ccc3oc4ccccc4c3c1n2-c1ccccc1 ; N#Cc1ccc(-n2c3ccccc3c3cc(C#N)ccc32)c(-c2ccc3c(c2)c2ccc4oc5ccccc5c4c2n3-c2ccccc2)c1 ; N#Cc1ccc(-c2ccc3c(c2)c2ccc4oc5ccccc5c4c2n3-c2ccccc2)c(-n2c3ccccc3c3cc(C#N)ccc32)c1]

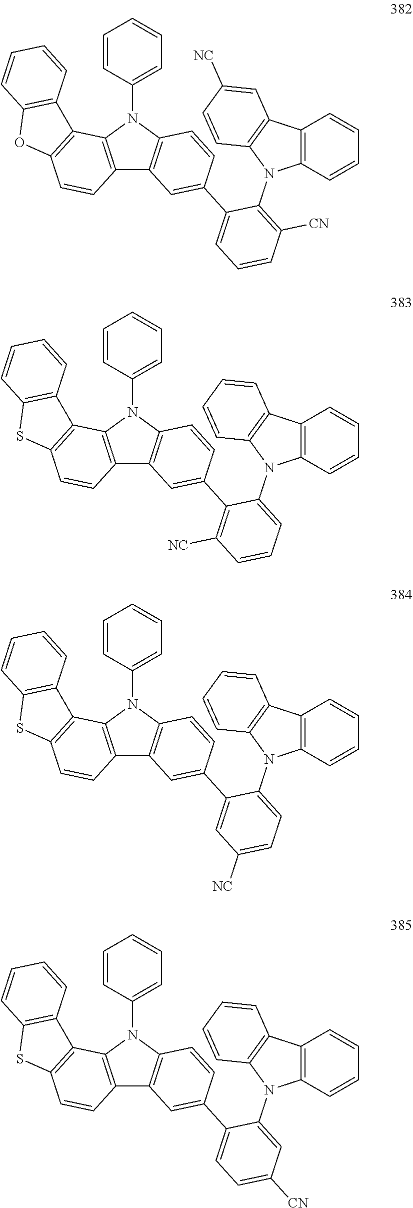 [SMILES: N#Cc1ccc2c(c1)c1ccccc1n2-c1c(C#N)cccc1-c1ccc2c(c1)c1ccc3oc4ccccc4c3c1n2-c1ccccc1 ; N#Cc1cccc(-n2c3ccccc3c3ccccc32)c1-c1ccc2c(c1)c1ccc3sc4ccccc4c3c1n2-c1ccccc1 ; N#Cc1ccc(-n2c3ccccc3c3ccccc32)c(-c2ccc3c(c2)c2ccc4sc5ccccc5c4c2n3-c2ccccc2)c1 ; N#Cc1ccc(-c2ccc3c(c2)c2ccc4sc5ccccc5c4c2n3-c2ccccc2)c(-n2c3ccccc3c3ccccc32)c1]

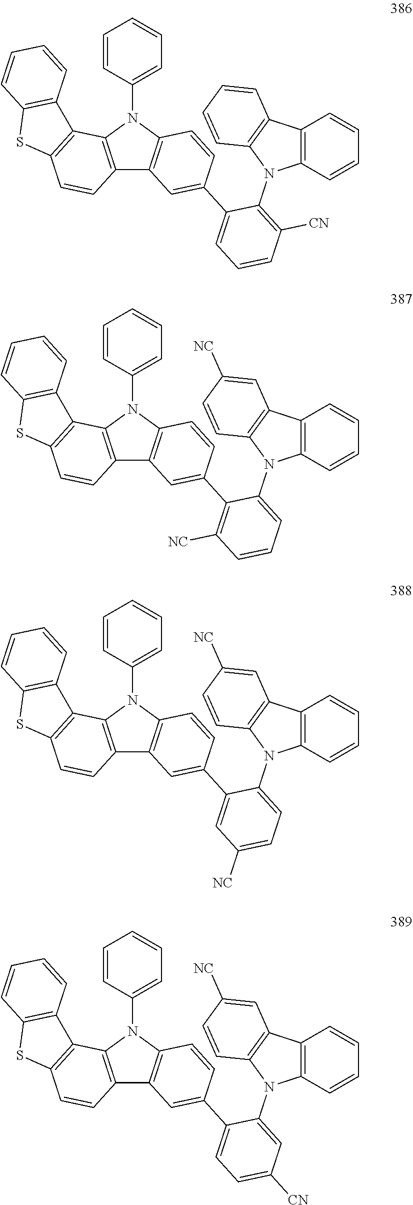 [SMILES: N#Cc1cccc(-c2ccc3c(c2)c2ccc4sc5ccccc5c4c2n3-c2ccccc2)c1-n1c2ccccc2c2ccccc21 ; N#Cc1ccc2c(c1)c1ccccc1n2-c1cccc(C#N)c1-c1ccc2c(c1)c1ccc3sc4ccccc4c3c1n2-c1ccccc1 ; N#Cc1ccc(-n2c3ccccc3c3cc(C#N)ccc32)c(-c2ccc3c(c2)c2ccc4sc5ccccc5c4c2n3-c2ccccc2)c1 ; N#Cc1ccc(-c2ccc3c(c2)c2ccc4sc5ccccc5c4c2n3-c2ccccc2)c(-n2c3ccccc3c3cc(C#N)ccc32)c1]

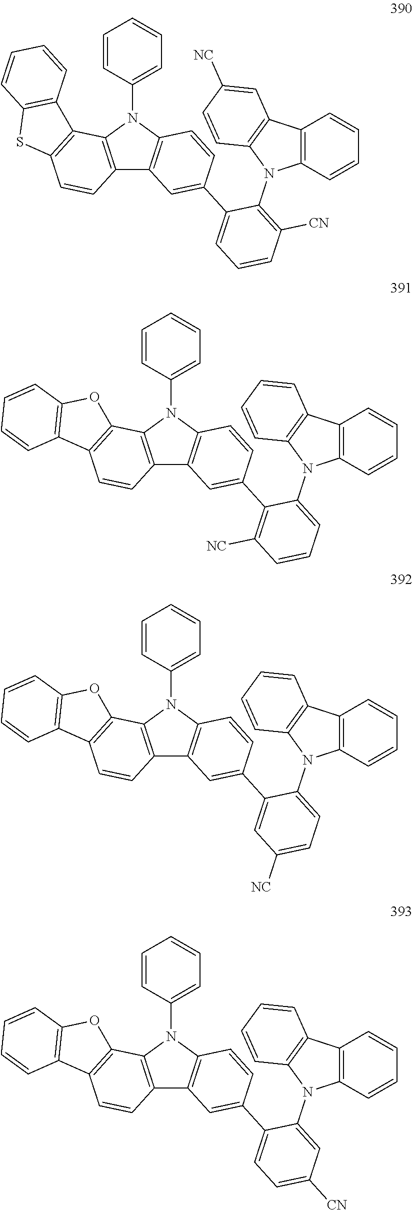 [SMILES: N#Cc1ccc2c(c1)c1ccccc1n2-c1c(C#N)cccc1-c1ccc2c(c1)c1ccc3sc4ccccc4c3c1n2-c1ccccc1 ; N#Cc1cccc(-n2c3ccccc3c3ccccc32)c1-c1ccc2c(c1)c1ccc3c4ccccc4oc3c1n2-c1ccccc1 ; N#Cc1ccc(-n2c3ccccc3c3ccccc32)c(-c2ccc3c(c2)c2ccc4c5ccccc5oc4c2n3-c2ccccc2)c1 ; N#Cc1ccc(-c2ccc3c(c2)c2ccc4c5ccccc5oc4c2n3-c2ccccc2)c(-n2c3ccccc3c3ccccc32)c1]

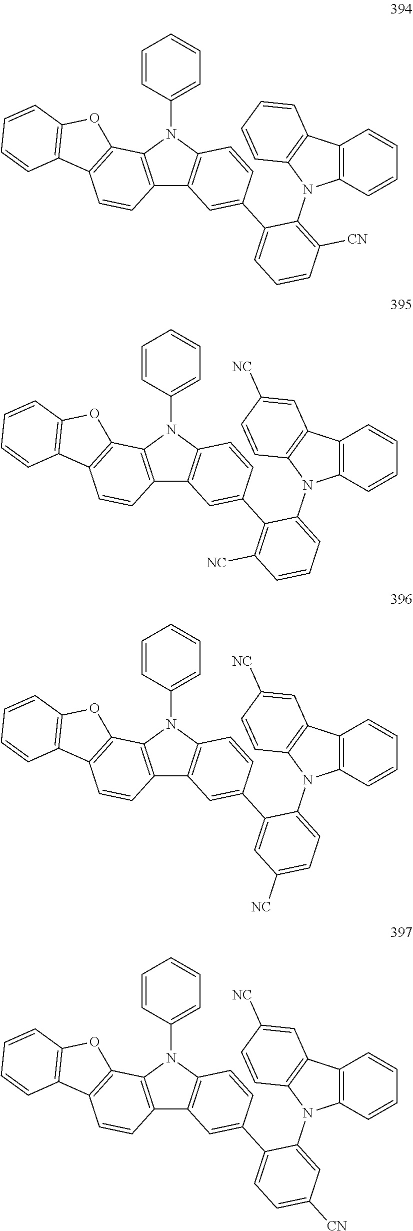 [SMILES: N#Cc1cccc(-c2ccc3c(c2)c2ccc4c5ccccc5oc4c2n3-c2ccccc2)c1-n1c2ccccc2c2ccccc21 ; N#Cc1ccc2c(c1)c1ccccc1n2-c1cccc(C#N)c1-c1ccc2c(c1)c1ccc3c4ccccc4oc3c1n2-c1ccccc1 ; N#Cc1ccc(-n2c3ccccc3c3cc(C#N)ccc32)c(-c2ccc3c(c2)c2ccc4c5ccccc5oc4c2n3-c2ccccc2)c1 ; N#Cc1ccc(-c2ccc3c(c2)c2ccc4c5ccccc5oc4c2n3-c2ccccc2)c(-n2c3ccccc3c3cc(C#N)ccc32)c1]

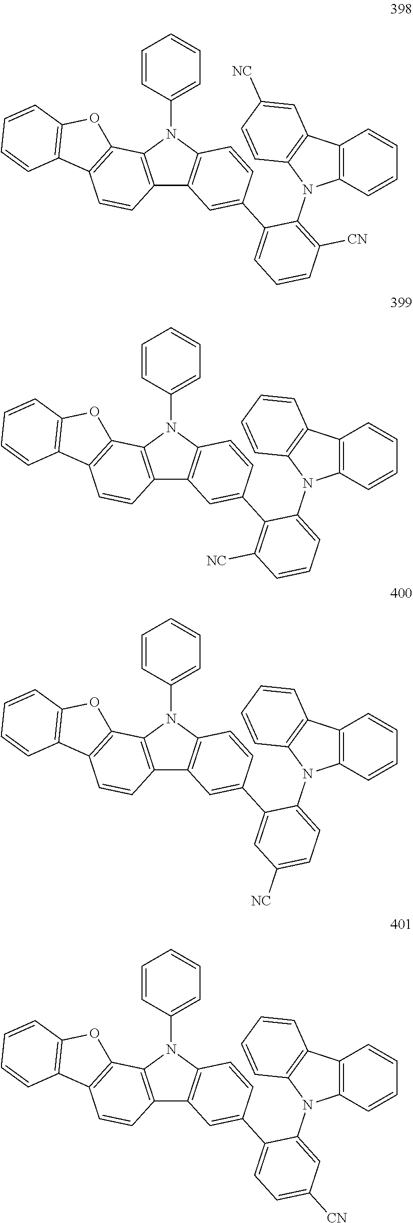 [SMILES: N#Cc1ccc2c(c1)c1ccccc1n2-c1c(C#N)cccc1-c1ccc2c(c1)c1ccc3c4ccccc4oc3c1n2-c1ccccc1 ; N#Cc1cccc(-n2c3ccccc3c3ccccc32)c1-c1ccc2c(c1)c1ccc3c4ccccc4oc3c1n2-c1ccccc1 ; N#Cc1ccc(-n2c3ccccc3c3ccccc32)c(-c2ccc3c(c2)c2ccc4c5ccccc5oc4c2n3-c2ccccc2)c1 ; N#Cc1ccc(-c2ccc3c(c2)c2ccc4c5ccccc5oc4c2n3-c2ccccc2)c(-n2c3ccccc3c3ccccc32)c1]

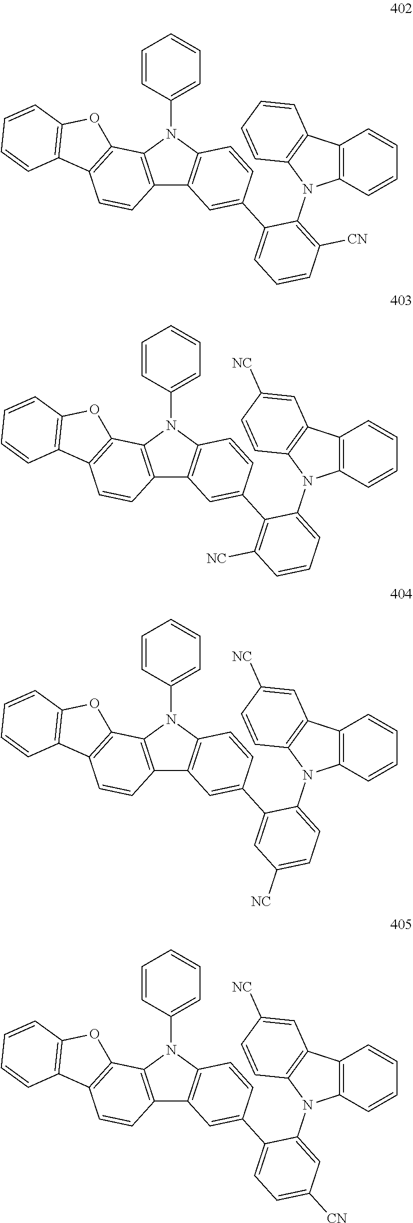 [SMILES: N#Cc1cccc(-c2ccc3c(c2)c2ccc4c5ccccc5oc4c2n3-c2ccccc2)c1-n1c2ccccc2c2ccccc21 ; N#Cc1ccc2c(c1)c1ccccc1n2-c1cccc(C#N)c1-c1ccc2c(c1)c1ccc3c4ccccc4oc3c1n2-c1ccccc1 ; N#Cc1ccc(-n2c3ccccc3c3cc(C#N)ccc32)c(-c2ccc3c(c2)c2ccc4c5ccccc5oc4c2n3-c2ccccc2)c1 ; N#Cc1ccc(-c2ccc3c(c2)c2ccc4c5ccccc5oc4c2n3-c2ccccc2)c(-n2c3ccccc3c3cc(C#N)ccc32)c1]

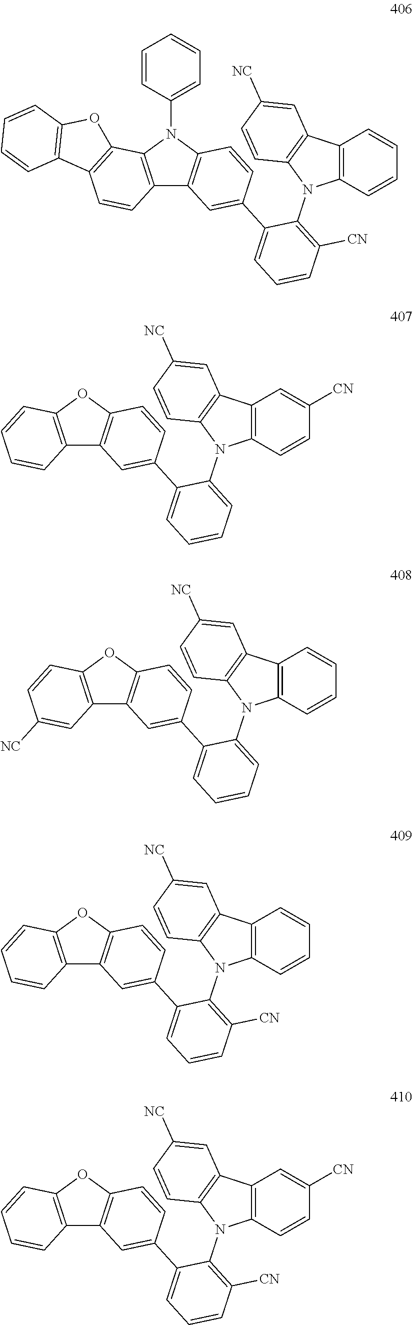

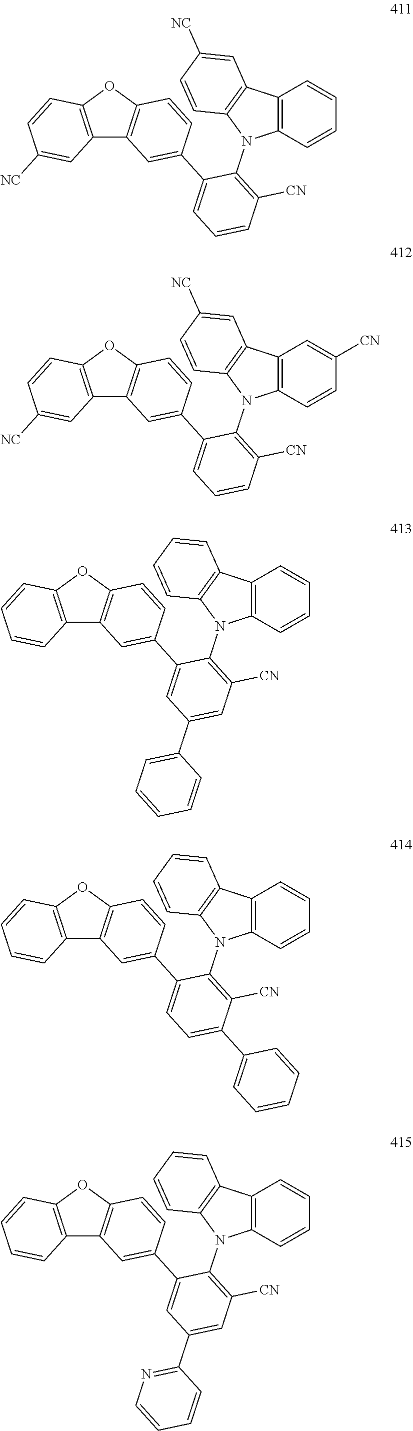

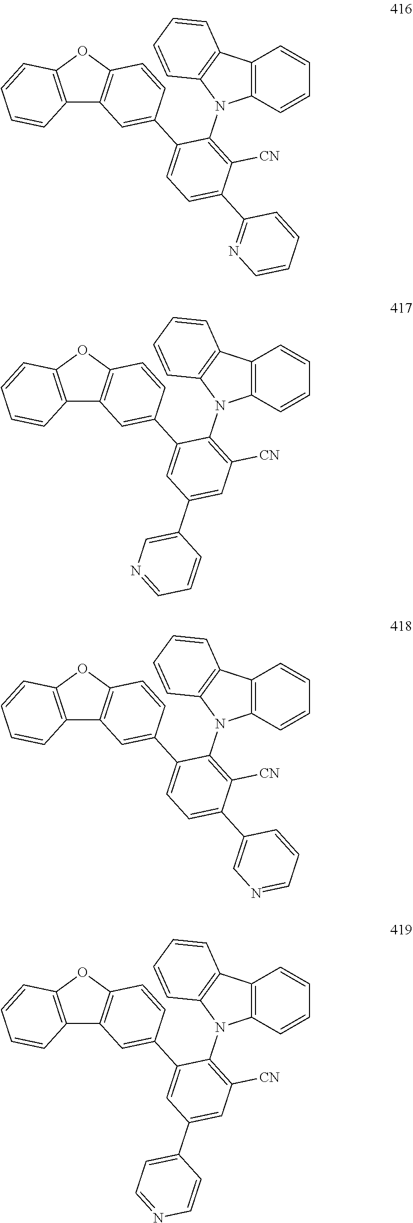

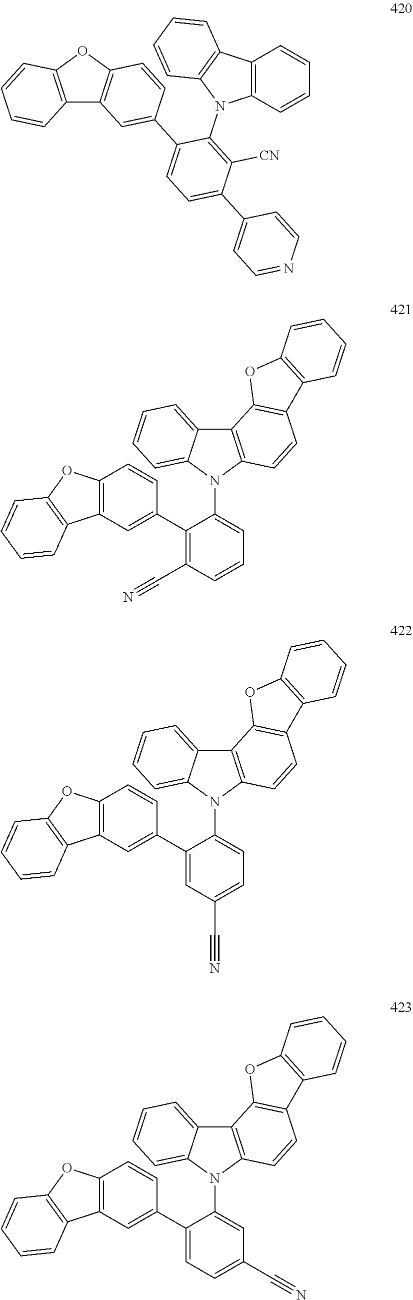 [SMILES: N#Cc1c(-c2ccncc2)ccc(-c2ccc3oc4ccccc4c3c2)c1-n1c2ccccc2c2ccccc21 ; N#Cc1cccc(-n2c3ccccc3c3c4oc5ccccc5c4ccc32)c1-c1ccc2oc3ccccc3c2c1 ; N#Cc1ccc(-n2c3ccccc3c3c4oc5ccccc5c4ccc32)c(-c2ccc3oc4ccccc4c3c2)c1 ; N#Cc1ccc(-c2ccc3oc4ccccc4c3c2)c(-n2c3ccccc3c3c4oc5ccccc5c4ccc32)c1]

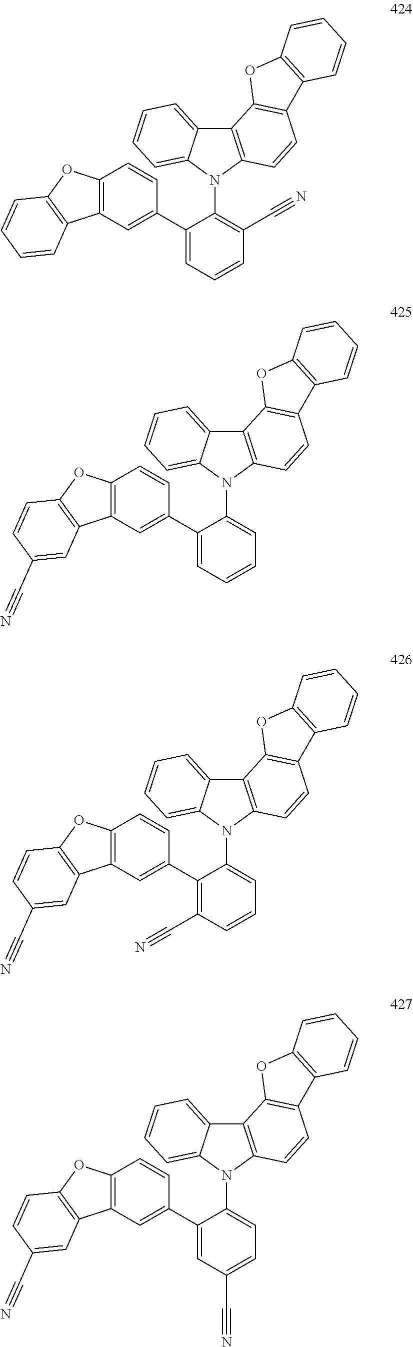 [SMILES: N#Cc1cccc(-c2ccc3oc4ccccc4c3c2)c1-n1c2ccccc2c2c3oc4ccccc4c3ccc21 ; N#Cc1ccc2oc3ccc(-c4ccccc4-n4c5ccccc5c5c6oc7ccccc7c6ccc54)cc3c2c1 ; N#Cc1ccc2oc3ccc(-c4c(C#N)cccc4-n4c5ccccc5c5c6oc7ccccc7c6ccc54)cc3c2c1 ; N#Cc1ccc(-n2c3ccccc3c3c4oc5ccccc5c4ccc32)c(-c2ccc3oc4ccc(C#N)cc4c3c2)c1]

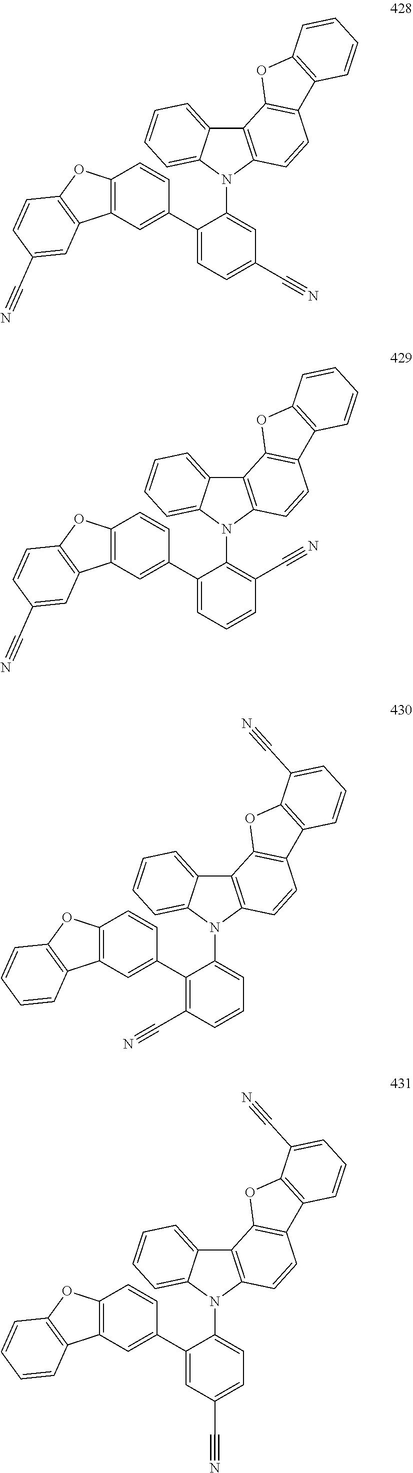 [SMILES: N#Cc1ccc(-c2ccc3oc4ccc(C#N)cc4c3c2)c(-n2c3ccccc3c3c4oc5ccccc5c4ccc32)c1 ; N#Cc1ccc2oc3ccc(-c4cccc(C#N)c4-n4c5ccccc5c5c6oc7ccccc7c6ccc54)cc3c2c1 ; N#Cc1cccc(-n2c3ccccc3c3c4oc5c(C#N)cccc5c4ccc32)c1-c1ccc2oc3ccccc3c2c1 ; N#Cc1ccc(-n2c3ccccc3c3c4oc5c(C#N)cccc5c4ccc32)c(-c2ccc3oc4ccccc4c3c2)c1]

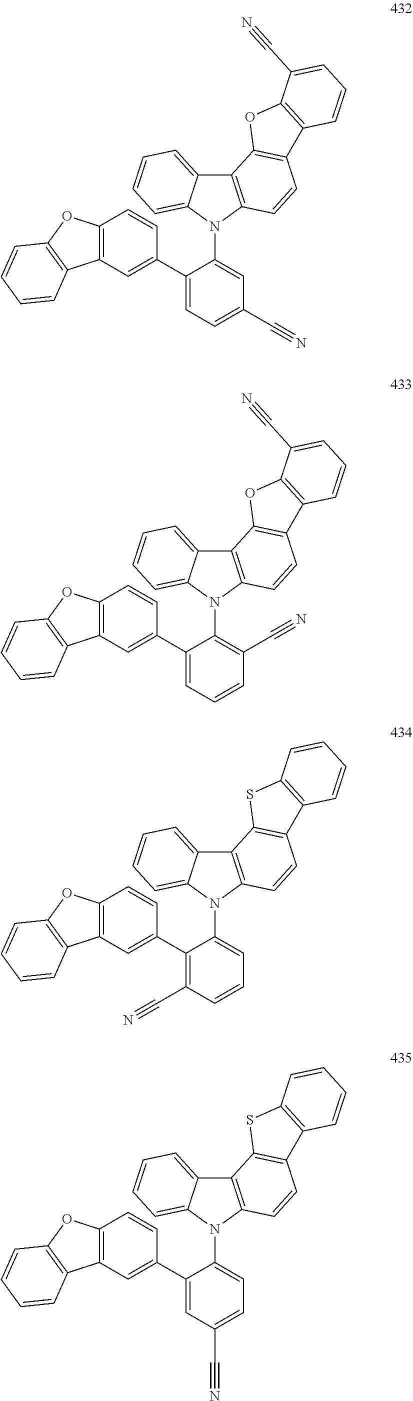 [SMILES: N#Cc1ccc(-c2ccc3oc4ccccc4c3c2)c(-n2c3ccccc3c3c4oc5c(C#N)cccc5c4ccc32)c1 ; N#Cc1cccc(-c2ccc3oc4ccccc4c3c2)c1-n1c2ccccc2c2c3oc4c(C#N)cccc4c3ccc21 ; N#Cc1cccc(-n2c3ccccc3c3c4sc5ccccc5c4ccc32)c1-c1ccc2oc3ccccc3c2c1 ; N#Cc1ccc(-n2c3ccccc3c3c4sc5ccccc5c4ccc32)c(-c2ccc3oc4ccccc4c3c2)c1]

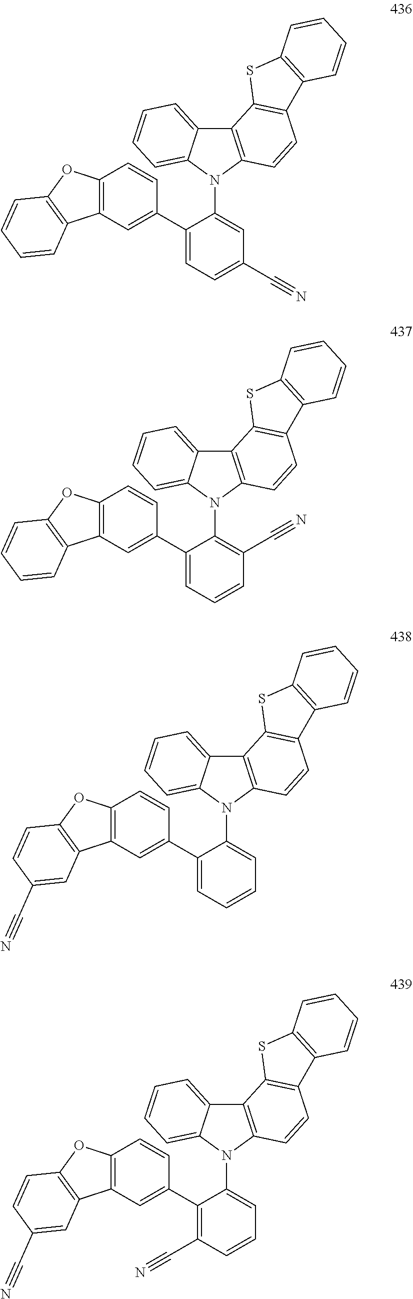 [SMILES: N#Cc1ccc(-c2ccc3oc4ccccc4c3c2)c(-n2c3ccccc3c3c4sc5ccccc5c4ccc32)c1 ; N#Cc1cccc(-c2ccc3oc4ccccc4c3c2)c1-n1c2ccccc2c2c3sc4ccccc4c3ccc21 ; N#Cc1ccc2oc3ccc(-c4ccccc4-n4c5ccccc5c5c6sc7ccccc7c6ccc54)cc3c2c1 ; N#Cc1ccc2oc3ccc(-c4c(C#N)cccc4-n4c5ccccc5c5c6sc7ccccc7c6ccc54)cc3c2c1]

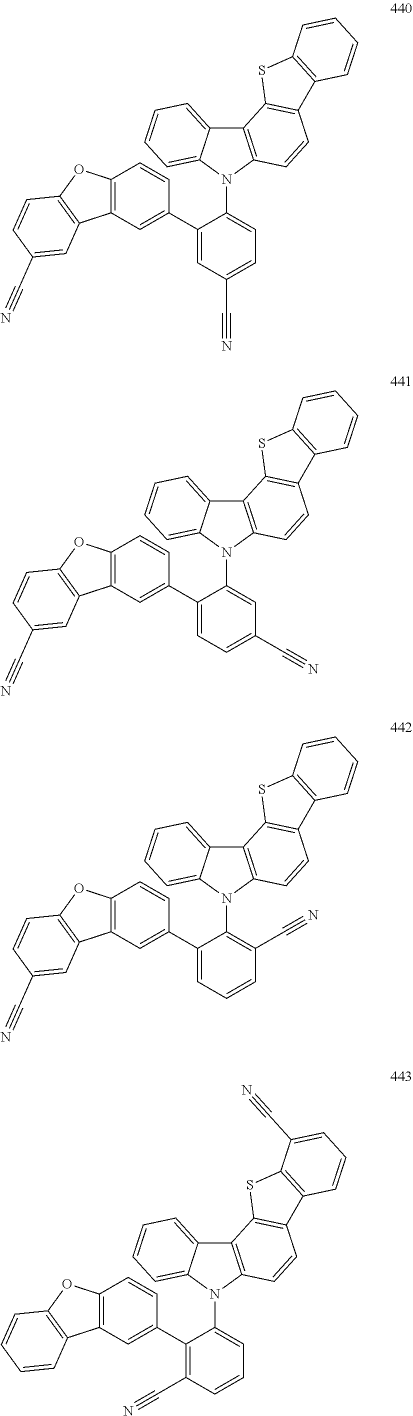 [SMILES: N#Cc1ccc(-n2c3ccccc3c3c4sc5ccccc5c4ccc32)c(-c2ccc3oc4ccc(C#N)cc4c3c2)c1 ; N#Cc1ccc(-c2ccc3oc4ccc(C#N)cc4c3c2)c(-n2c3ccccc3c3c4sc5ccccc5c4ccc32)c1 ; N#Cc1ccc2oc3ccc(-c4cccc(C#N)c4-n4c5ccccc5c5c6sc7ccccc7c6ccc54)cc3c2c1 ; N#Cc1cccc(-n2c3ccccc3c3c4sc5c(C#N)cccc5c4ccc32)c1-c1ccc2oc3ccccc3c2c1]

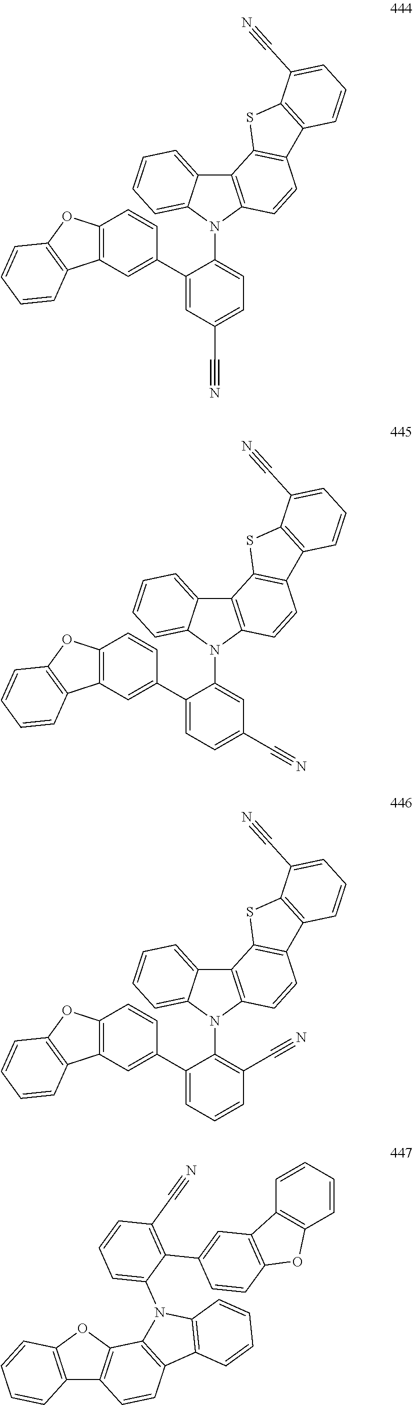 [SMILES: N#Cc1ccc(-n2c3ccccc3c3c4sc5c(C#N)cccc5c4ccc32)c(-c2ccc3oc4ccccc4c3c2)c1 ; N#Cc1ccc(-c2ccc3oc4ccccc4c3c2)c(-n2c3ccccc3c3c4sc5c(C#N)cccc5c4ccc32)c1 ; N#Cc1cccc(-c2ccc3oc4ccccc4c3c2)c1-n1c2ccccc2c2c3sc4c(C#N)cccc4c3ccc21 ; N#Cc1cccc(-n2c3ccccc3c3ccc4c5ccccc5oc4c32)c1-c1ccc2oc3ccccc3c2c1]

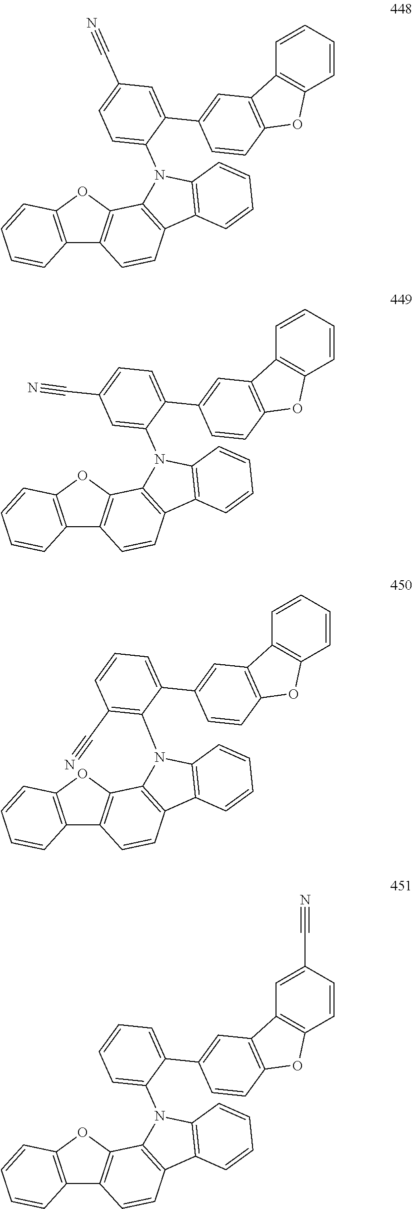 [SMILES: N#Cc1ccc(-n2c3ccccc3c3ccc4c5ccccc5oc4c32)c(-c2ccc3oc4ccccc4c3c2)c1 ; N#Cc1ccc(-c2ccc3oc4ccccc4c3c2)c(-n2c3ccccc3c3ccc4c5ccccc5oc4c32)c1 ; N#Cc1cccc(-c2ccc3oc4ccccc4c3c2)c1-n1c2ccccc2c2ccc3c4ccccc4oc3c21 ; N#Cc1ccc2oc3ccc(-c4ccccc4-n4c5ccccc5c5ccc6c7ccccc7oc6c54)cc3c2c1]

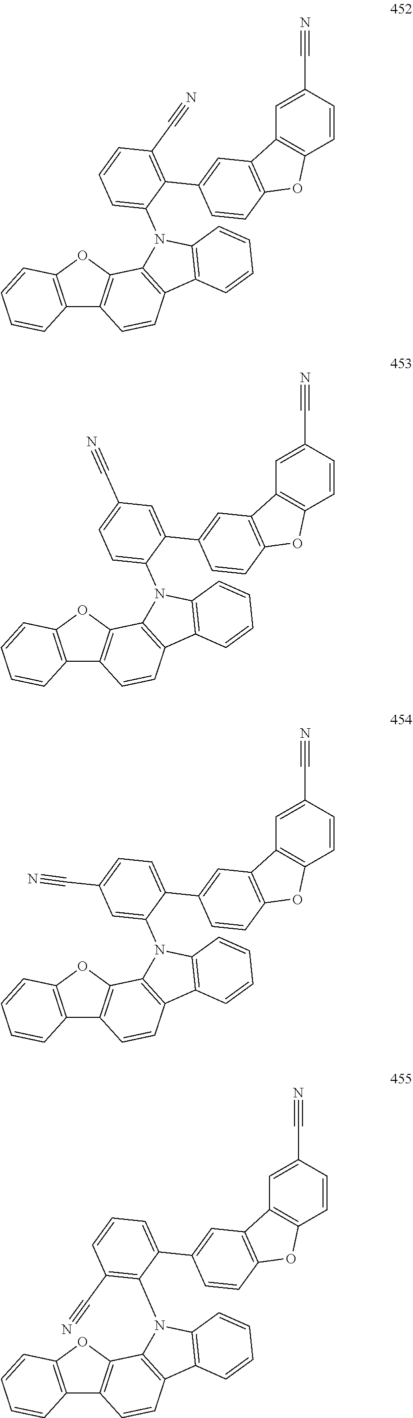 [SMILES: N#Cc1ccc2oc3ccc(-c4c(C#N)cccc4-n4c5ccccc5c5ccc6c7ccccc7oc6c54)cc3c2c1 ; N#Cc1ccc(-n2c3ccccc3c3ccc4c5ccccc5oc4c32)c(-c2ccc3oc4ccc(C#N)cc4c3c2)c1 ; N#Cc1ccc(-c2ccc3oc4ccc(C#N)cc4c3c2)c(-n2c3ccccc3c3ccc4c5ccccc5oc4c32)c1 ; N#Cc1ccc2oc3ccc(-c4cccc(C#N)c4-n4c5ccccc5c5ccc6c7ccccc7oc6c54)cc3c2c1]

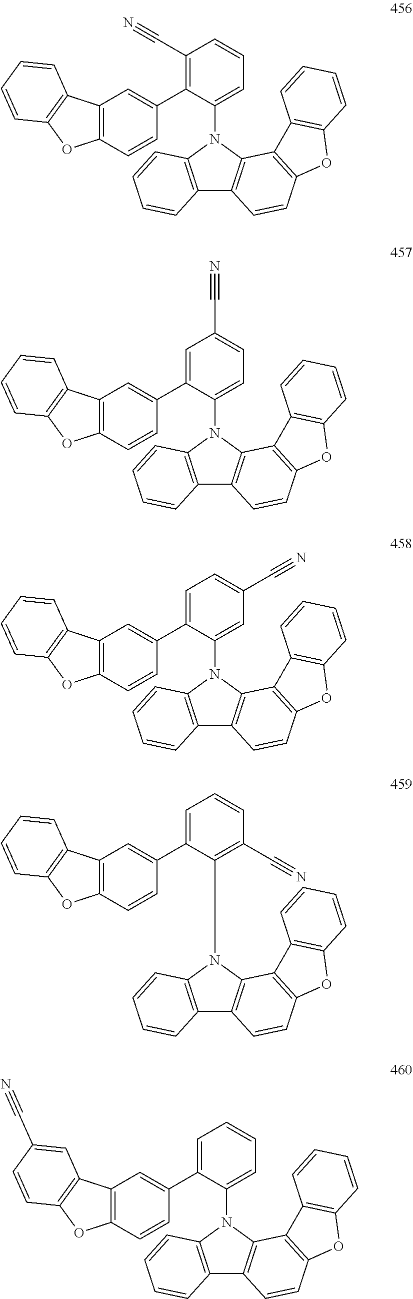 [SMILES: N#Cc1cccc(-n2c3ccccc3c3ccc4oc5ccccc5c4c32)c1-c1ccc2oc3ccccc3c2c1 ; N#Cc1ccc(-n2c3ccccc3c3ccc4oc5ccccc5c4c32)c(-c2ccc3oc4ccccc4c3c2)c1 ; N#Cc1ccc(-c2ccc3oc4ccccc4c3c2)c(-n2c3ccccc3c3ccc4oc5ccccc5c4c32)c1 ; N#Cc1cccc(-c2ccc3oc4ccccc4c3c2)c1-n1c2ccccc2c2ccc3oc4ccccc4c3c21 ; N#Cc1ccc2oc3ccc(-c4ccccc4-n4c5ccccc5c5ccc6oc7ccccc7c6c54)cc3c2c1]

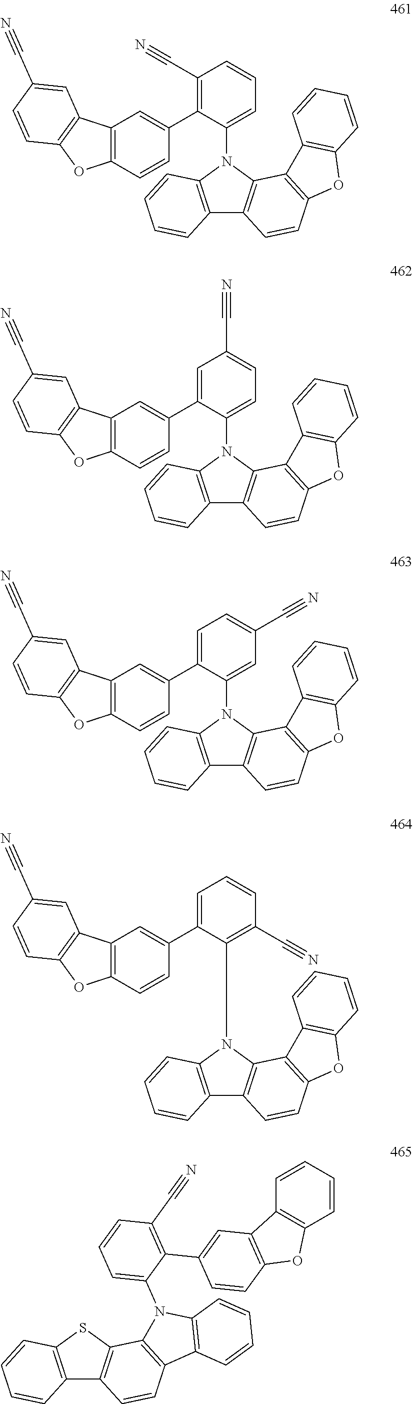 [SMILES: N#Cc1ccc2oc3ccc(-c4c(C#N)cccc4-n4c5ccccc5c5ccc6oc7ccccc7c6c54)cc3c2c1 ; N#Cc1ccc(-n2c3ccccc3c3ccc4oc5ccccc5c4c32)c(-c2ccc3oc4ccc(C#N)cc4c3c2)c1 ; N#Cc1ccc(-c2ccc3oc4ccc(C#N)cc4c3c2)c(-n2c3ccccc3c3ccc4oc5ccccc5c4c32)c1 ; N#Cc1ccc2oc3ccc(-c4cccc(C#N)c4-n4c5ccccc5c5ccc6oc7ccccc7c6c54)cc3c2c1 ; N#Cc1cccc(-n2c3ccccc3c3ccc4c5ccccc5sc4c32)c1-c1ccc2oc3ccccc3c2c1]

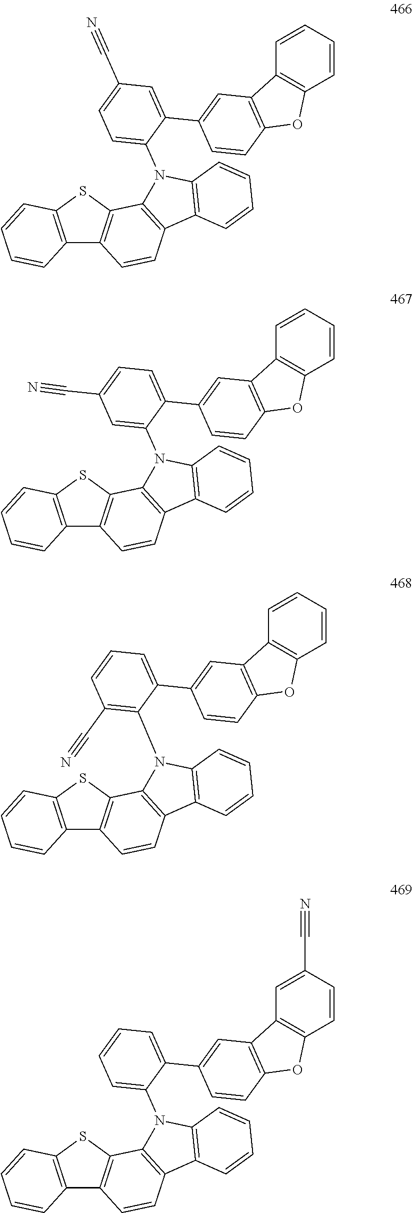 [SMILES: N#Cc1ccc(-n2c3ccccc3c3ccc4c5ccccc5sc4c32)c(-c2ccc3oc4ccccc4c3c2)c1 ; N#Cc1ccc(-c2ccc3oc4ccccc4c3c2)c(-n2c3ccccc3c3ccc4c5ccccc5sc4c32)c1 ; N#Cc1cccc(-c2ccc3oc4ccccc4c3c2)c1-n1c2ccccc2c2ccc3c4ccccc4sc3c21 ; N#Cc1ccc2oc3ccc(-c4ccccc4-n4c5ccccc5c5ccc6c7ccccc7sc6c54)cc3c2c1]

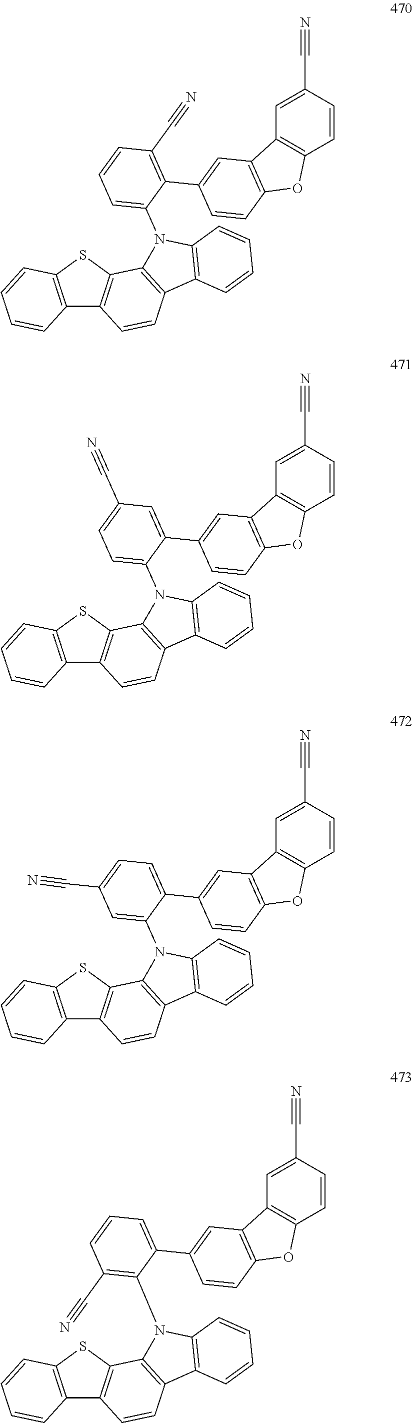 [SMILES: N#Cc1ccc2oc3ccc(-c4c(C#N)cccc4-n4c5ccccc5c5ccc6c7ccccc7sc6c54)cc3c2c1 ; N#Cc1ccc(-n2c3ccccc3c3ccc4c5ccccc5sc4c32)c(-c2ccc3oc4ccc(C#N)cc4c3c2)c1 ; N#Cc1ccc(-c2ccc3oc4ccc(C#N)cc4c3c2)c(-n2c3ccccc3c3ccc4c5ccccc5sc4c32)c1 ; N#Cc1ccc2oc3ccc(-c4cccc(C#N)c4-n4c5ccccc5c5ccc6c7ccccc7sc6c54)cc3c2c1]

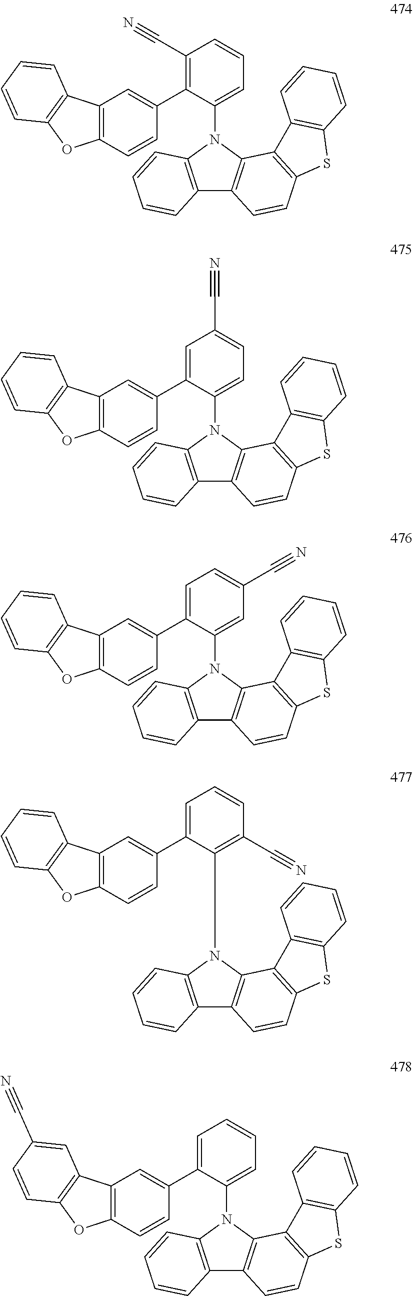 [SMILES: N#Cc1cccc(-n2c3ccccc3c3ccc4sc5ccccc5c4c32)c1-c1ccc2oc3ccccc3c2c1 ; N#Cc1ccc(-n2c3ccccc3c3ccc4sc5ccccc5c4c32)c(-c2ccc3oc4ccccc4c3c2)c1 ; N#Cc1ccc(-c2ccc3oc4ccccc4c3c2)c(-n2c3ccccc3c3ccc4sc5ccccc5c4c32)c1 ; N#Cc1cccc(-c2ccc3oc4ccccc4c3c2)c1-n1c2ccccc2c2ccc3sc4ccccc4c3c21 ; N#Cc1ccc2oc3ccc(-c4ccccc4-n4c5ccccc5c5ccc6sc7ccccc7c6c54)cc3c2c1]

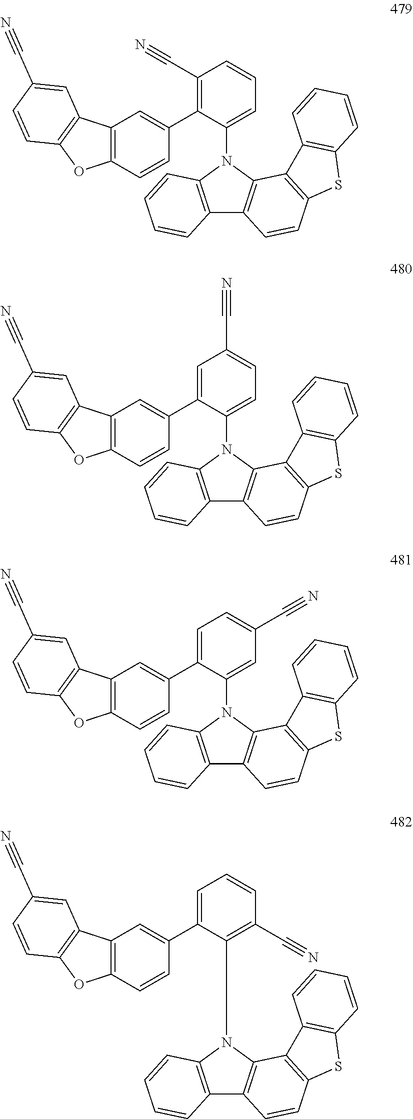 [SMILES: N#Cc1ccc2oc3ccc(-c4c(C#N)cccc4-n4c5ccccc5c5ccc6sc7ccccc7c6c54)cc3c2c1 ; N#Cc1ccc(-n2c3ccccc3c3ccc4sc5ccccc5c4c32)c(-c2ccc3oc4ccc(C#N)cc4c3c2)c1 ; N#Cc1ccc(-c2ccc3oc4ccc(C#N)cc4c3c2)c(-n2c3ccccc3c3ccc4sc5ccccc5c4c32)c1 ; N#Cc1ccc2oc3ccc(-c4cccc(C#N)c4-n4c5ccccc5c5ccc6sc7ccccc7c6c54)cc3c2c1]

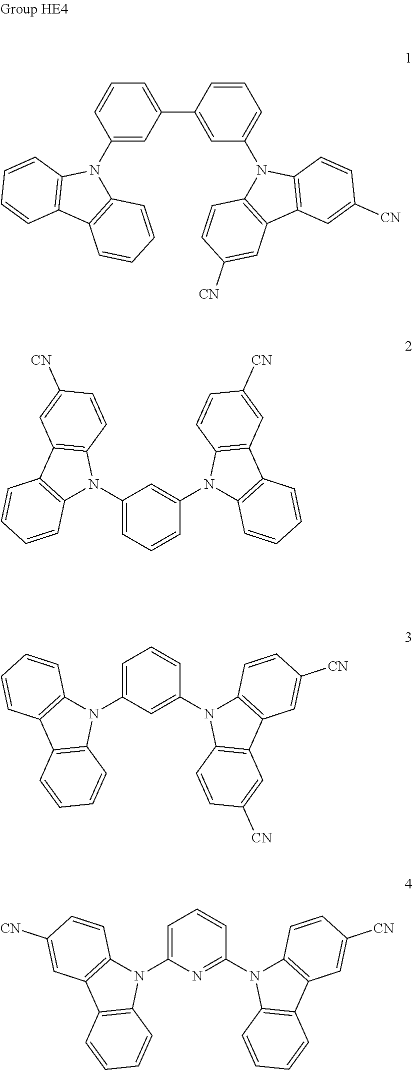

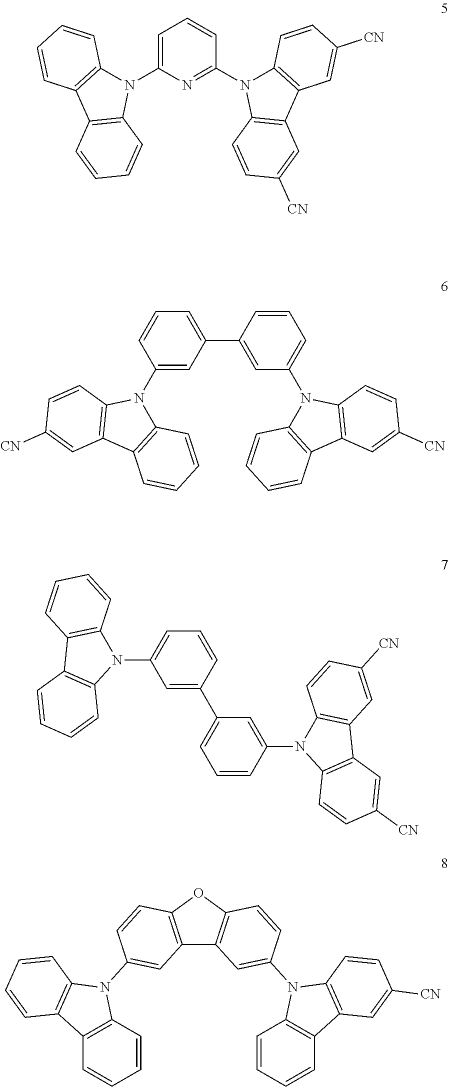

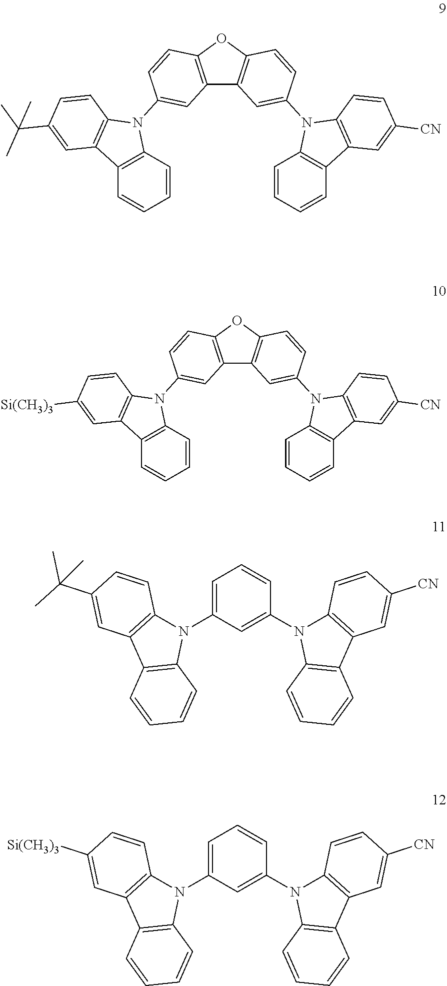

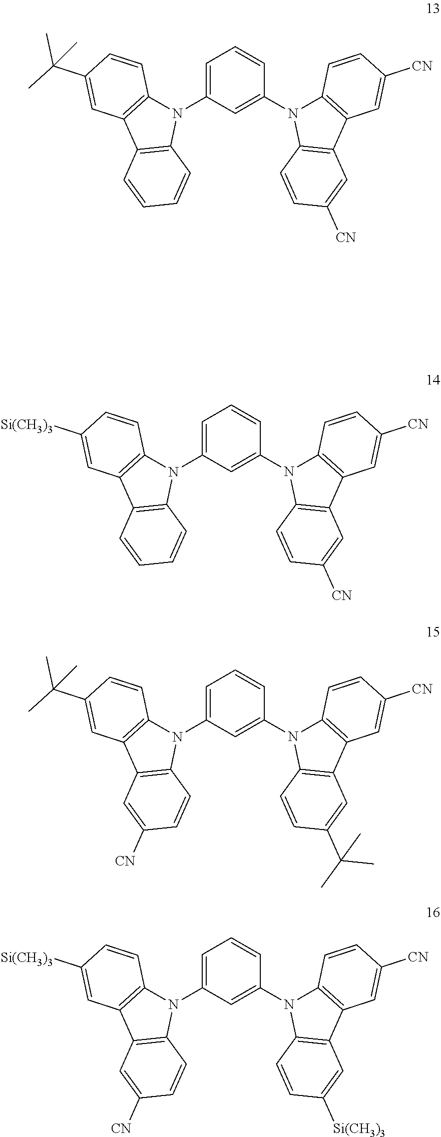

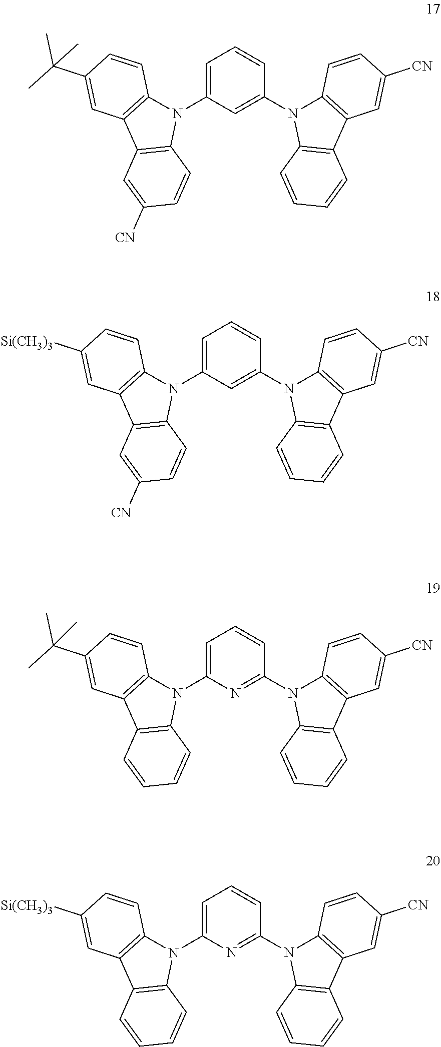

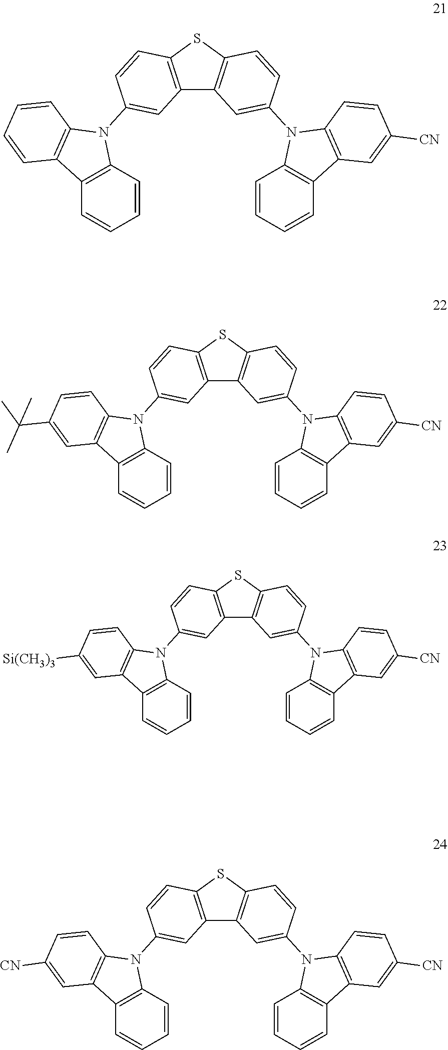

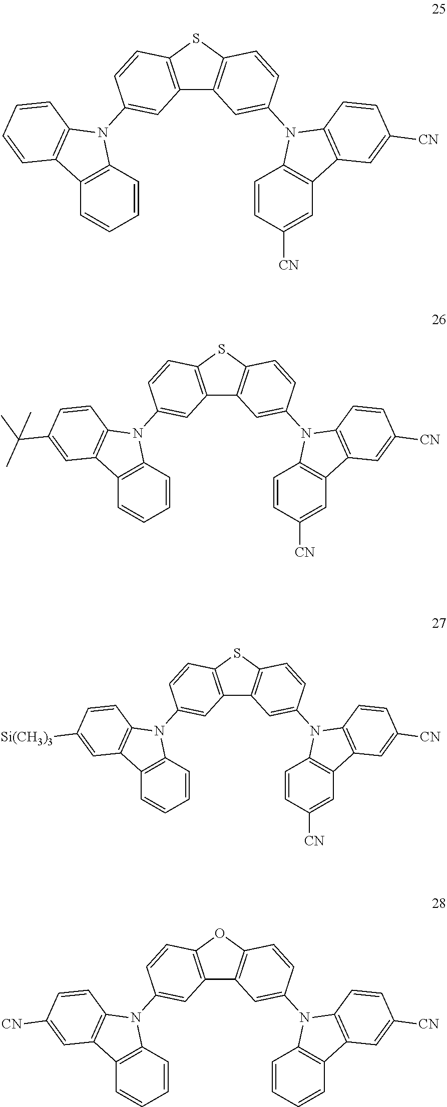

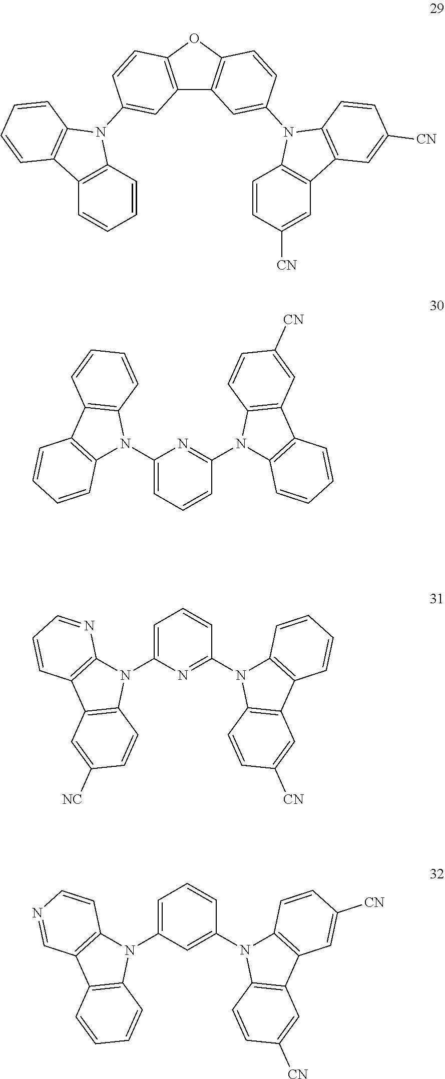 [SMILES: N#Cc1ccc2c(c1)c1cc(C#N)ccc1n2-c1ccc2oc3ccc(-n4c5ccccc5c5ccccc54)cc3c2c1 ; N#Cc1ccc2c(c1)c1ccccc1n2-c1cccc(-n2c3ccccc3c3ccccc32)n1 ; N#Cc1ccc2c(c1)c1ccccc1n2-c1cccc(-n2c3ccc(C#N)cc3c3cccnc32)n1 ; N#Cc1ccc2c(c1)c1cc(C#N)ccc1n2-c1cccc(-n2c3ccccc3c3cnccc32)c1]

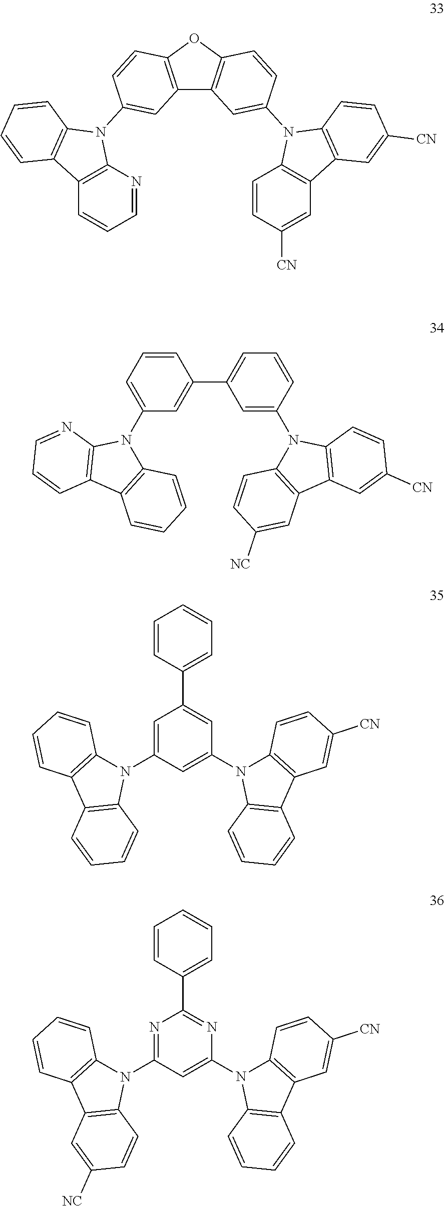 [SMILES: N#Cc1ccc2c(c1)c1cc(C#N)ccc1n2-c1ccc2oc3ccc(-n4c5ccccc5c5cccnc54)cc3c2c1 ; N#Cc1ccc2c(c1)c1cc(C#N)ccc1n2-c1cccc(-c2cccc(-n3c4ccccc4c4cccnc43)c2)c1 ; N#Cc1ccc2c(c1)c1ccccc1n2-c1cc(-c2ccccc2)cc(-n2c3ccccc3c3ccccc32)c1 ; N#Cc1ccc2c(c1)c1ccccc1n2-c1cc(-n2c3ccccc3c3cc(C#N)ccc32)nc(-c2ccccc2)n1]

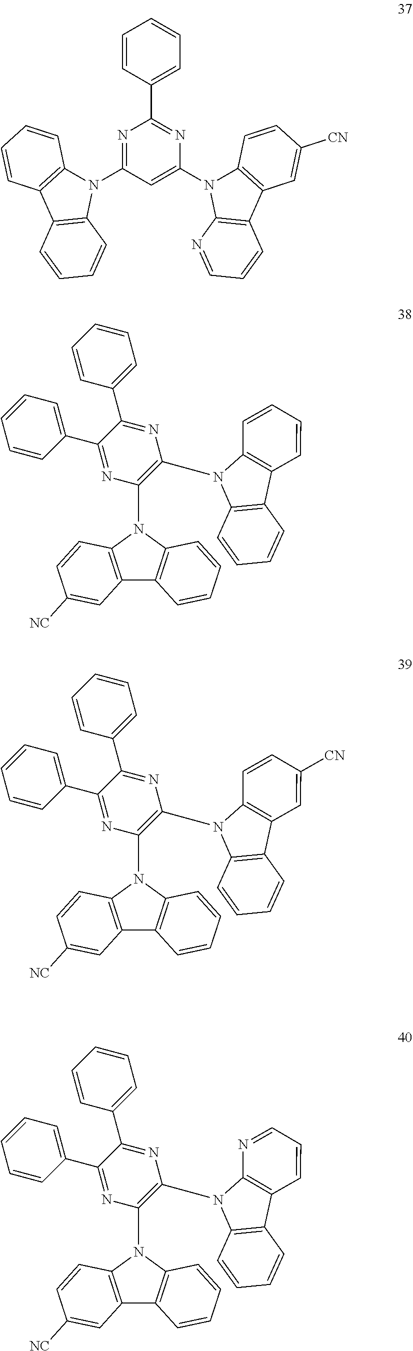 [SMILES: N#Cc1ccc2c(c1)c1cccnc1n2-c1cc(-n2c3ccccc3c3ccccc32)nc(-c2ccccc2)n1 ; N#Cc1ccc2c(c1)c1ccccc1n2-c1nc(-c2ccccc2)c(-c2ccccc2)nc1-n1c2ccccc2c2ccccc21 ; N#Cc1ccc2c(c1)c1ccccc1n2-c1nc(-c2ccccc2)c(-c2ccccc2)nc1-n1c2ccccc2c2cc(C#N)ccc21 ; N#Cc1ccc2c(c1)c1ccccc1n2-c1nc(-c2ccccc2)c(-c2ccccc2)nc1-n1c2ccccc2c2cccnc21]

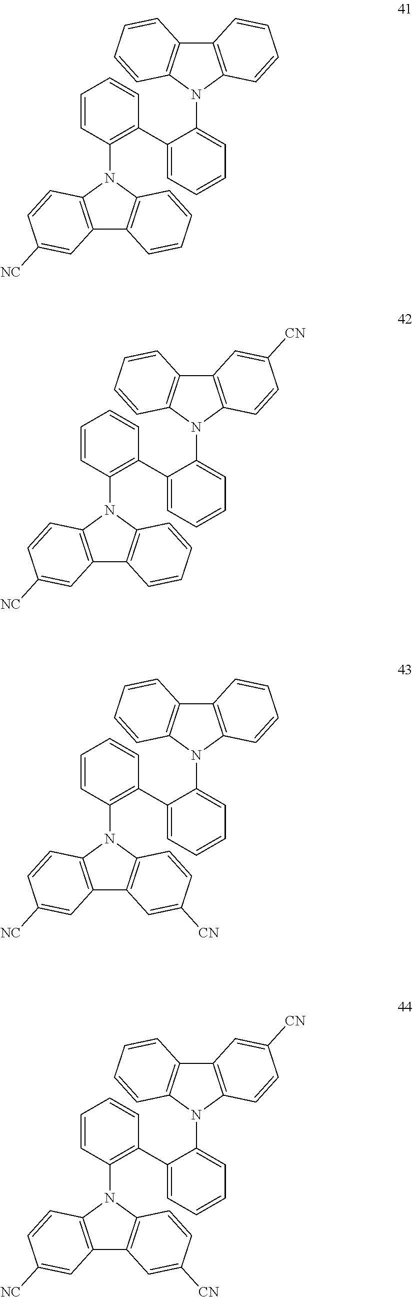

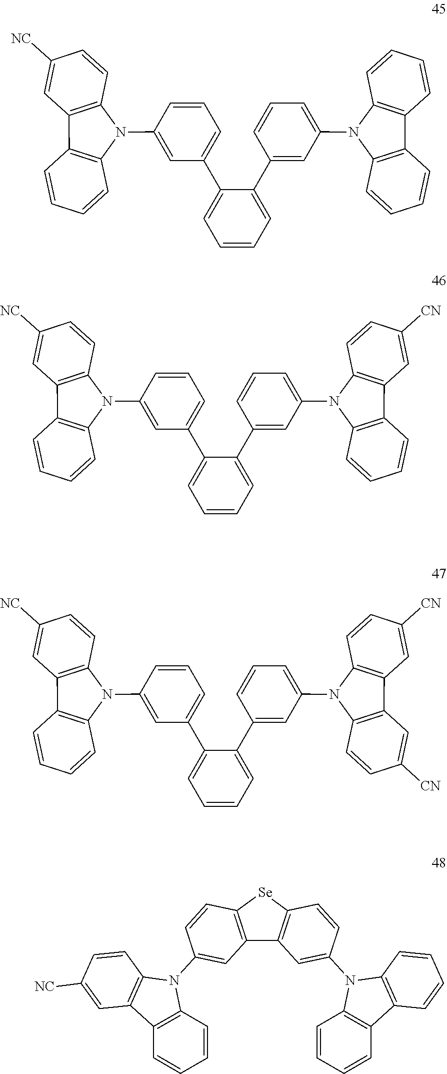 [SMILES: N#Cc1ccc2c(c1)c1ccccc1n2-c1cccc(-c2ccccc2-c2cccc(-n3c4ccccc4c4ccccc43)c2)c1 ; N#Cc1ccc2c(c1)c1ccccc1n2-c1cccc(-c2ccccc2-c2cccc(-n3c4ccccc4c4cc(C#N)ccc43)c2)c1 ; N#Cc1ccc2c(c1)c1ccccc1n2-c1cccc(-c2ccccc2-c2cccc(-n3c4ccc(C#N)cc4c4cc(C#N)ccc43)c2)c1 ; N#Cc1ccc2c(c1)c1ccccc1n2-c1ccc2[se]c3ccc(-n4c5ccccc5c5ccccc54)cc3c2c1]

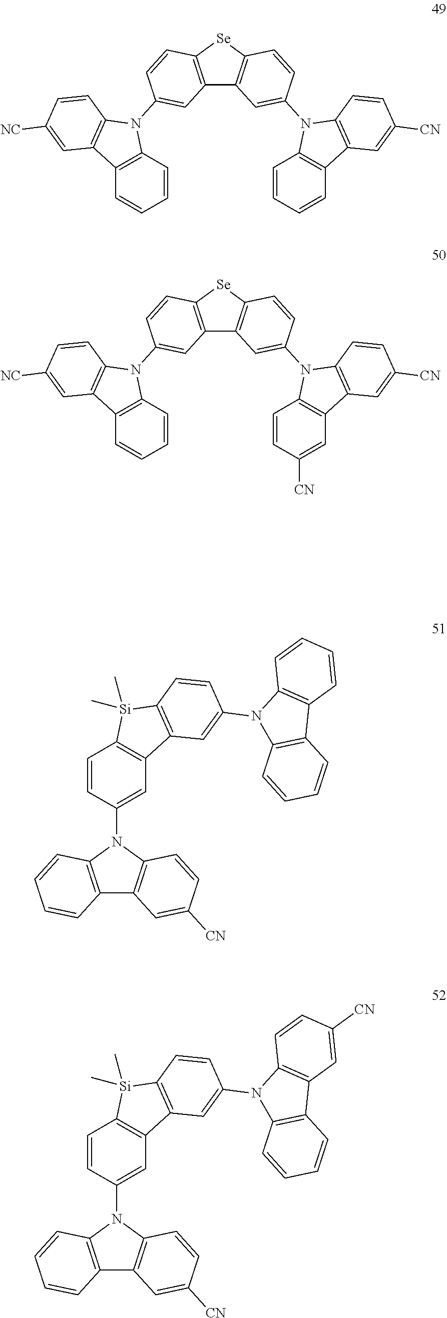 [SMILES: N#Cc1ccc2c(c1)c1ccccc1n2-c1ccc2[se]c3ccc(-n4c5ccccc5c5cc(C#N)ccc54)cc3c2c1 ; N#Cc1ccc2c(c1)c1ccccc1n2-c1ccc2[se]c3ccc(-n4c5ccc(C#N)cc5c5cc(C#N)ccc54)cc3c2c1 ; C[Si]1(C)c2ccc(-n3c4ccccc4c4ccccc43)cc2-c2cc(-n3c4ccccc4c4cc(C#N)ccc43)ccc21 ; C[Si]1(C)c2ccc(-n3c4ccccc4c4cc(C#N)ccc43)cc2-c2cc(-n3c4ccccc4c4cc(C#N)ccc43)ccc21]

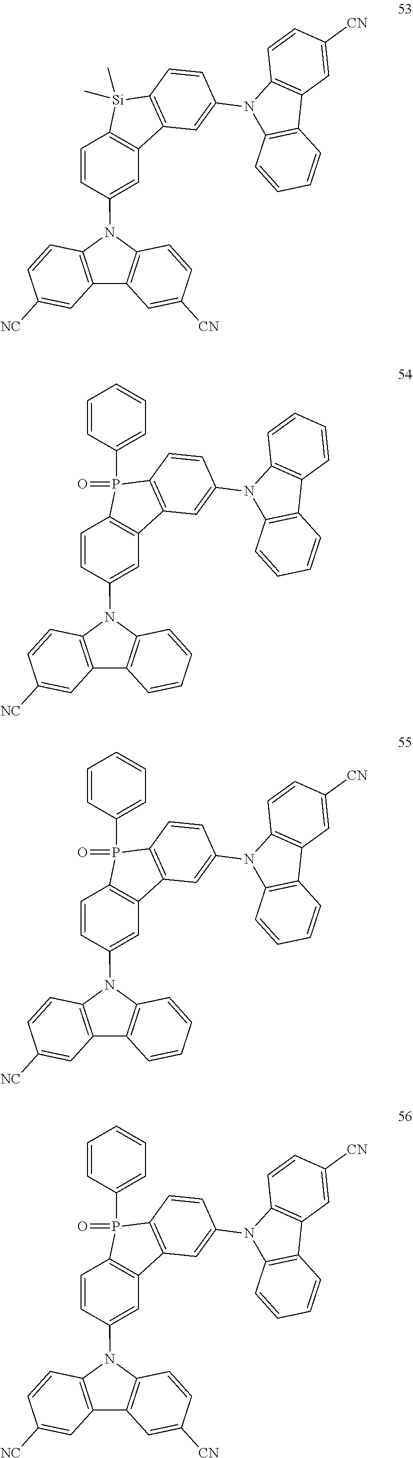 [SMILES: C[Si]1(C)c2ccc(-n3c4ccccc4c4cc(C#N)ccc43)cc2-c2cc(-n3c4ccc(C#N)cc4c4cc(C#N)ccc43)ccc21 ; N#Cc1ccc2c(c1)c1ccccc1n2-c1ccc2c(c1)-c1cc(-n3c4ccccc4c4ccccc43)ccc1P2(=O)c1ccccc1 ; N#Cc1ccc2c(c1)c1ccccc1n2-c1ccc2c(c1)-c1cc(-n3c4ccccc4c4cc(C#N)ccc43)ccc1P2(=O)c1ccccc1 ; N#Cc1ccc2c(c1)c1ccccc1n2-c1ccc2c(c1)-c1cc(-n3c4ccc(C#N)cc4c4cc(C#N)ccc43)ccc1P2(=O)c1ccccc1]

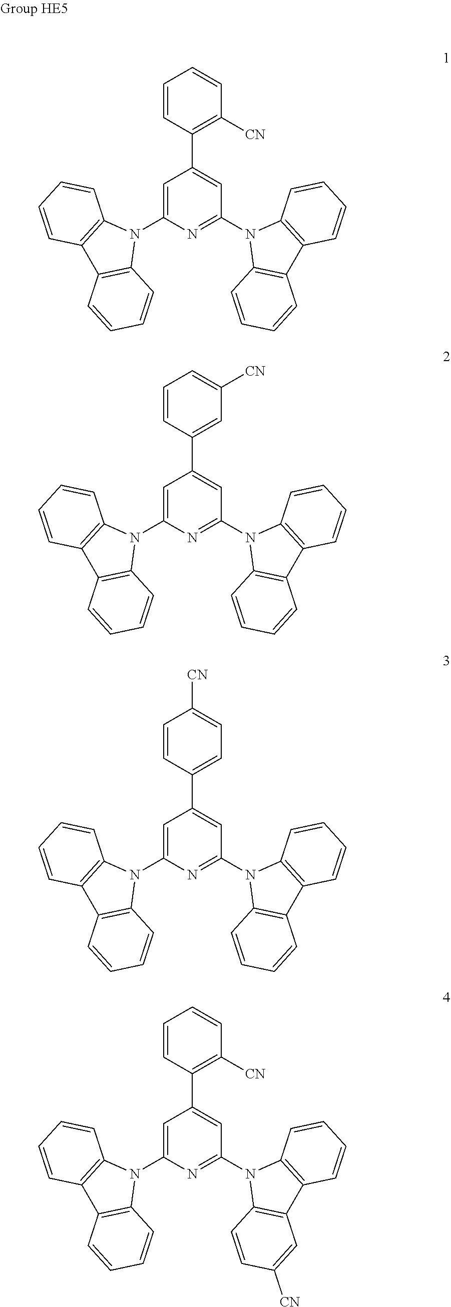

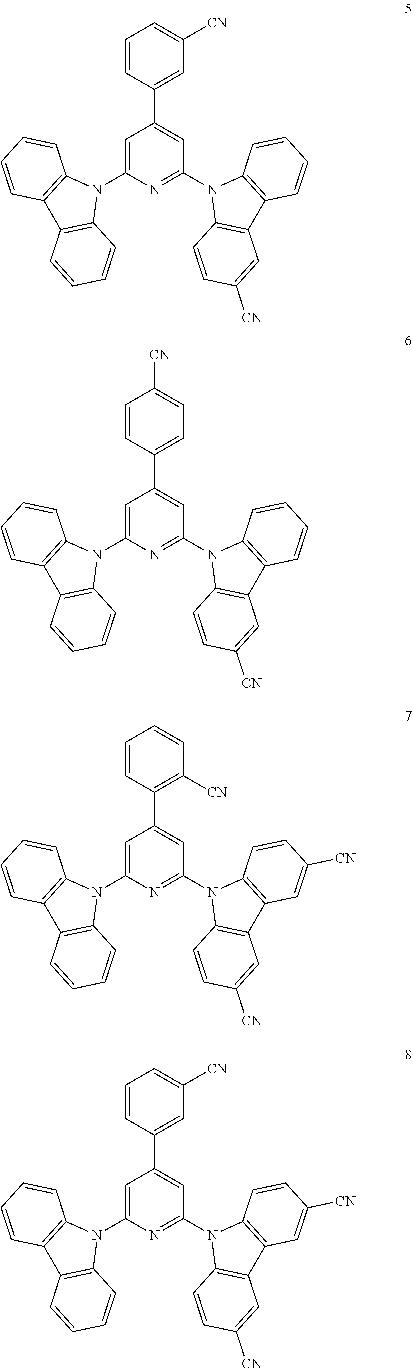 [SMILES: N#Cc1cccc(-c2cc(-n3c4ccccc4c4ccccc43)nc(-n3c4ccccc4c4cc(C#N)ccc43)c2)c1 ; N#Cc1ccc(-c2cc(-n3c4ccccc4c4ccccc43)nc(-n3c4ccccc4c4cc(C#N)ccc43)c2)cc1 ; N#Cc1ccc2c(c1)c1cc(C#N)ccc1n2-c1cc(-c2ccccc2C#N)cc(-n2c3ccccc3c3ccccc32)n1 ; N#Cc1cccc(-c2cc(-n3c4ccccc4c4ccccc43)nc(-n3c4ccc(C#N)cc4c4cc(C#N)ccc43)c2)c1]

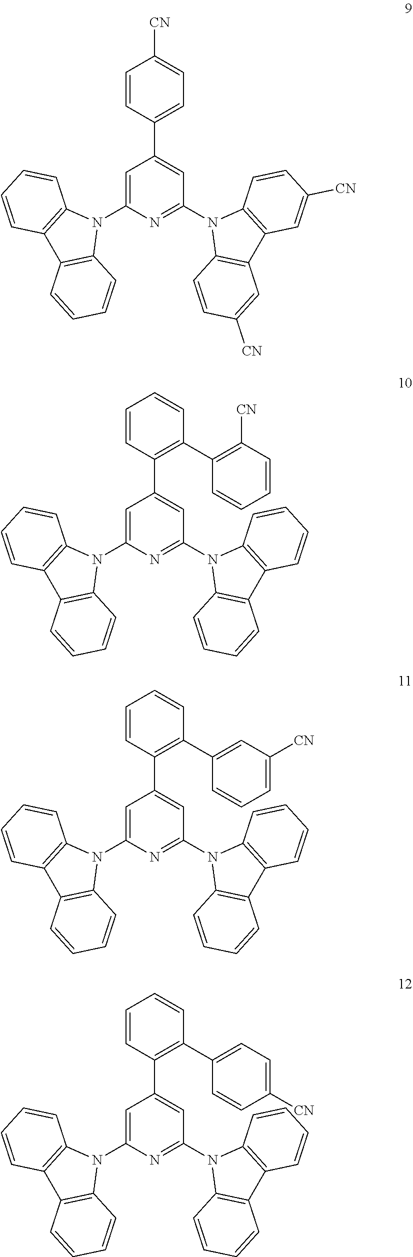 [SMILES: N#Cc1ccc(-c2cc(-n3c4ccccc4c4ccccc43)nc(-n3c4ccc(C#N)cc4c4cc(C#N)ccc43)c2)cc1 ; N#Cc1ccccc1-c1ccccc1-c1cc(-n2c3ccccc3c3ccccc32)nc(-n2c3ccccc3c3ccccc32)c1 ; N#Cc1cccc(-c2ccccc2-c2cc(-n3c4ccccc4c4ccccc43)nc(-n3c4ccccc4c4ccccc43)c2)c1 ; N#Cc1ccc(-c2ccccc2-c2cc(-n3c4ccccc4c4ccccc43)nc(-n3c4ccccc4c4ccccc43)c2)cc1]

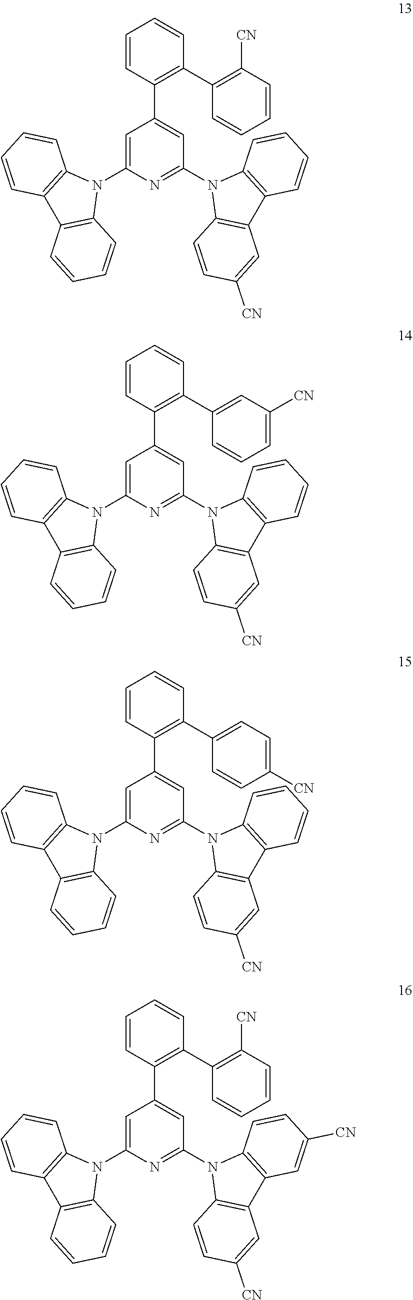 [SMILES: N#Cc1ccc2c(c1)c1ccccc1n2-c1cc(-c2ccccc2-c2ccccc2C#N)cc(-n2c3ccccc3c3ccccc32)n1 ; N#Cc1cccc(-c2ccccc2-c2cc(-n3c4ccccc4c4ccccc43)nc(-n3c4ccccc4c4cc(C#N)ccc43)c2)c1 ; N#Cc1ccc(-c2ccccc2-c2cc(-n3c4ccccc4c4ccccc43)nc(-n3c4ccccc4c4cc(C#N)ccc43)c2)cc1 ; N#Cc1ccc2c(c1)c1cc(C#N)ccc1n2-c1cc(-c2ccccc2-c2ccccc2C#N)cc(-n2c3ccccc3c3ccccc32)n1]

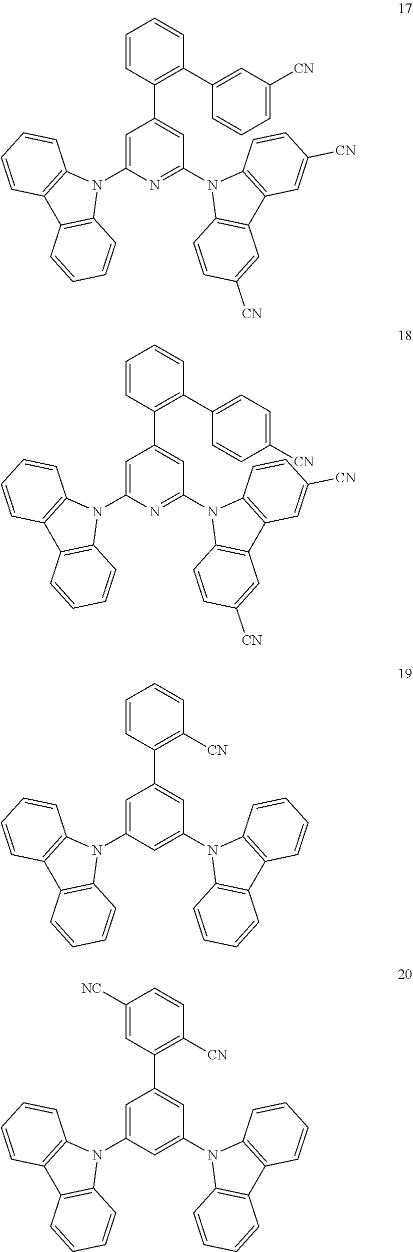 [SMILES: N#Cc1cccc(-c2ccccc2-c2cc(-n3c4ccccc4c4ccccc43)nc(-n3c4ccc(C#N)cc4c4cc(C#N)ccc43)c2)c1 ; N#Cc1ccc(-c2ccccc2-c2cc(-n3c4ccccc4c4ccccc43)nc(-n3c4ccc(C#N)cc4c4cc(C#N)ccc43)c2)cc1 ; N#Cc1ccccc1-c1cc(-n2c3ccccc3c3ccccc32)cc(-n2c3ccccc3c3ccccc32)c1 ; N#Cc1ccc(C#N)c(-c2cc(-n3c4ccccc4c4ccccc43)cc(-n3c4ccccc4c4ccccc43)c2)c1]

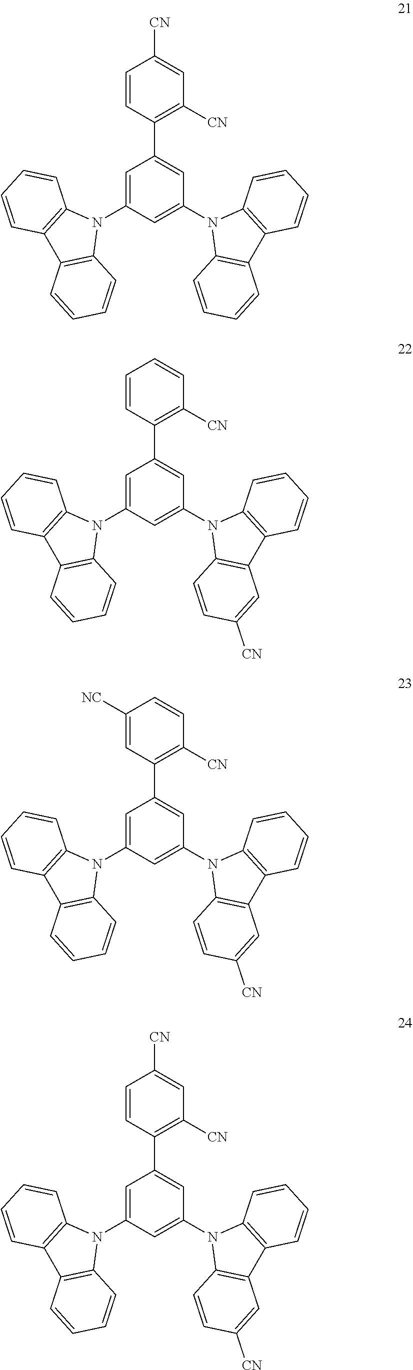 [SMILES: N#Cc1ccc(-c2cc(-n3c4ccccc4c4ccccc43)cc(-n3c4ccccc4c4ccccc43)c2)c(C#N)c1 ; N#Cc1ccc2c(c1)c1ccccc1n2-c1cc(-c2ccccc2C#N)cc(-n2c3ccccc3c3ccccc32)c1 ; N#Cc1ccc(C#N)c(-c2cc(-n3c4ccccc4c4ccccc43)cc(-n3c4ccccc4c4cc(C#N)ccc43)c2)c1 ; N#Cc1ccc(-c2cc(-n3c4ccccc4c4ccccc43)cc(-n3c4ccccc4c4cc(C#N)ccc43)c2)c(C#N)c1]

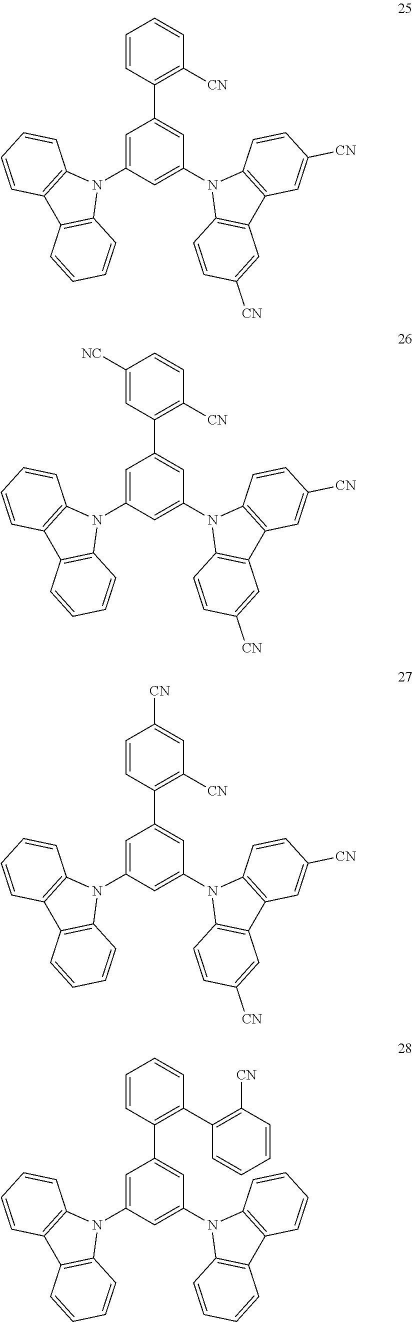 [SMILES: N#Cc1ccc2c(c1)c1cc(C#N)ccc1n2-c1cc(-c2ccccc2C#N)cc(-n2c3ccccc3c3ccccc32)c1 ; N#Cc1ccc(C#N)c(-c2cc(-n3c4ccccc4c4ccccc43)cc(-n3c4ccc(C#N)cc4c4cc(C#N)ccc43)c2)c1 ; N#Cc1ccc(-c2cc(-n3c4ccccc4c4ccccc43)cc(-n3c4ccc(C#N)cc4c4cc(C#N)ccc43)c2)c(C#N)c1 ; N#Cc1ccccc1-c1ccccc1-c1cc(-n2c3ccccc3c3ccccc32)cc(-n2c3ccccc3c3ccccc32)c1]

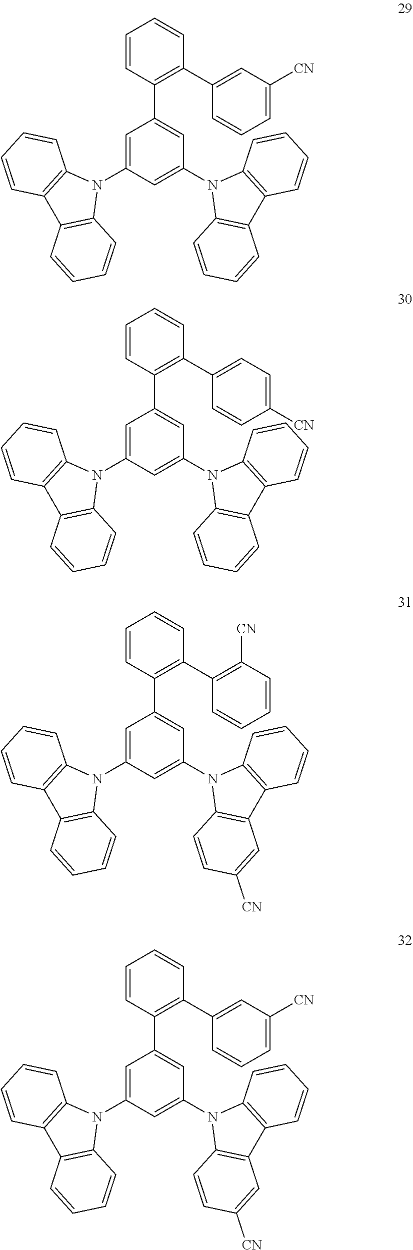 [SMILES: N#Cc1cccc(-c2ccccc2-c2cc(-n3c4ccccc4c4ccccc43)cc(-n3c4ccccc4c4ccccc43)c2)c1 ; N#Cc1ccc(-c2ccccc2-c2cc(-n3c4ccccc4c4ccccc43)cc(-n3c4ccccc4c4ccccc43)c2)cc1 ; N#Cc1ccc2c(c1)c1ccccc1n2-c1cc(-c2ccccc2-c2ccccc2C#N)cc(-n2c3ccccc3c3ccccc32)c1 ; N#Cc1cccc(-c2ccccc2-c2cc(-n3c4ccccc4c4ccccc43)cc(-n3c4ccccc4c4cc(C#N)ccc43)c2)c1]

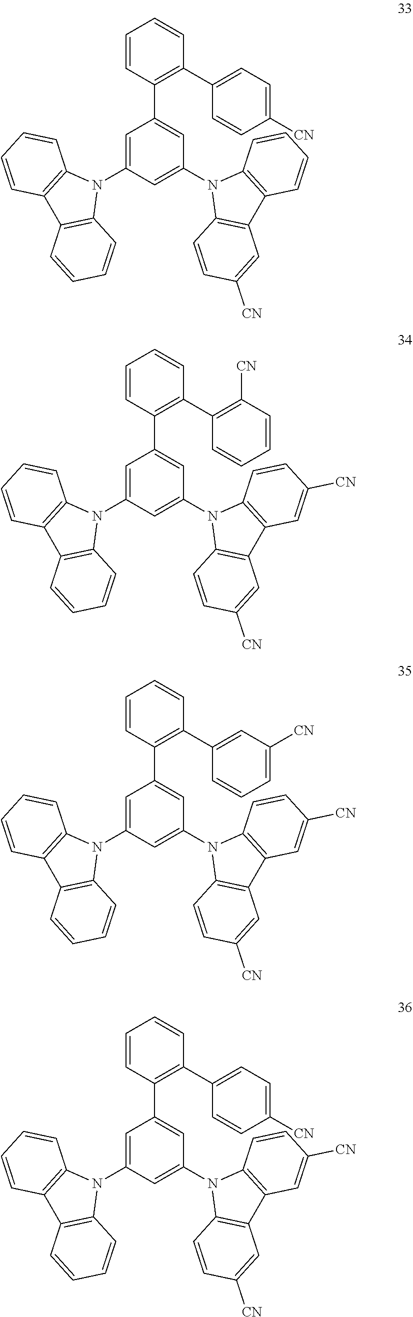 [SMILES: N#Cc1ccc(-c2ccccc2-c2cc(-n3c4ccccc4c4ccccc43)cc(-n3c4ccccc4c4cc(C#N)ccc43)c2)cc1 ; N#Cc1ccc2c(c1)c1cc(C#N)ccc1n2-c1cc(-c2ccccc2-c2ccccc2C#N)cc(-n2c3ccccc3c3ccccc32)c1 ; N#Cc1cccc(-c2ccccc2-c2cc(-n3c4ccccc4c4ccccc43)cc(-n3c4ccc(C#N)cc4c4cc(C#N)ccc43)c2)c1 ; N#Cc1ccc(-c2ccccc2-c2cc(-n3c4ccccc4c4ccccc43)cc(-n3c4ccc(C#N)cc4c4cc(C#N)ccc43)c2)cc1]

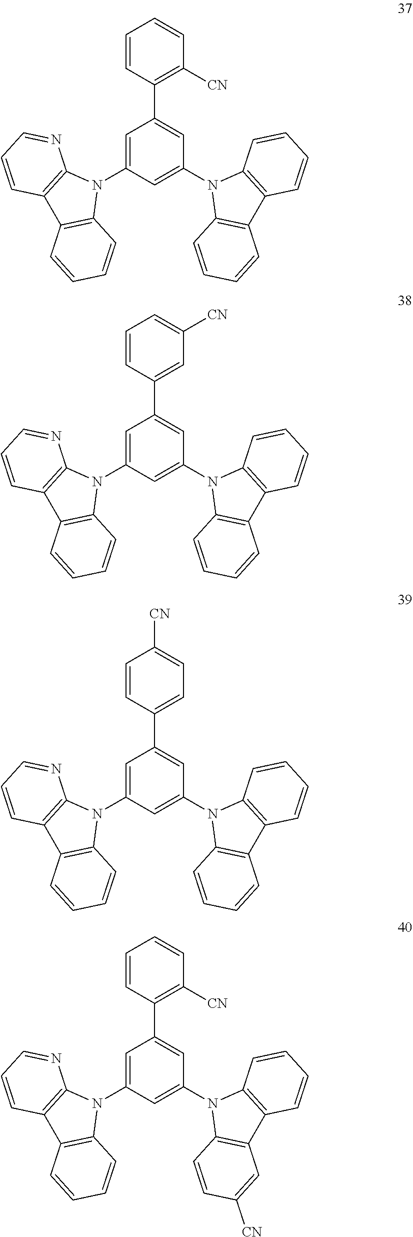

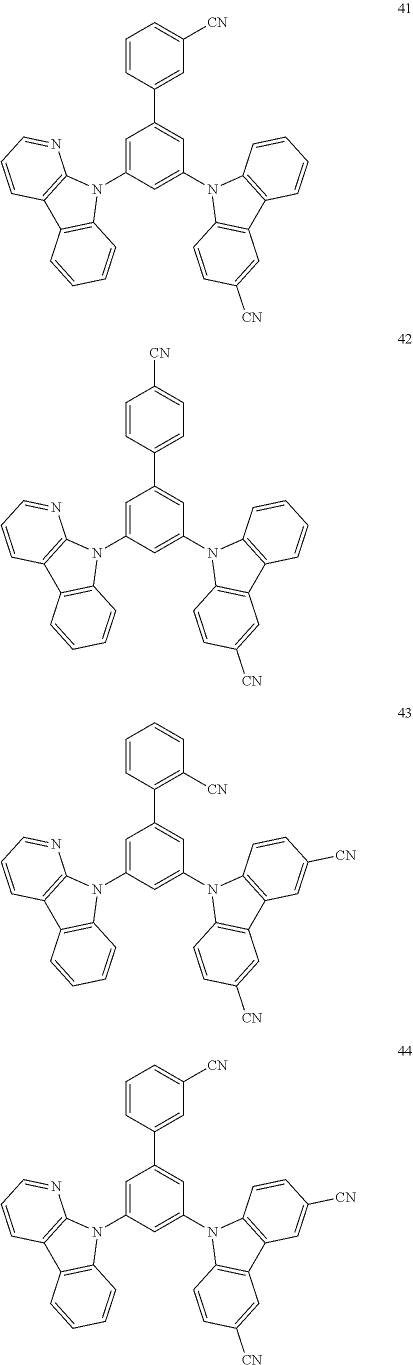 [SMILES: N#Cc1cccc(-c2cc(-n3c4ccccc4c4cc(C#N)ccc43)cc(-n3c4ccccc4c4cccnc43)c2)c1 ; N#Cc1ccc(-c2cc(-n3c4ccccc4c4cc(C#N)ccc43)cc(-n3c4ccccc4c4cccnc43)c2)cc1 ; N#Cc1ccc2c(c1)c1cc(C#N)ccc1n2-c1cc(-c2ccccc2C#N)cc(-n2c3ccccc3c3cccnc32)c1 ; N#Cc1cccc(-c2cc(-n3c4ccc(C#N)cc4c4cc(C#N)ccc43)cc(-n3c4ccccc4c4cccnc43)c2)c1]

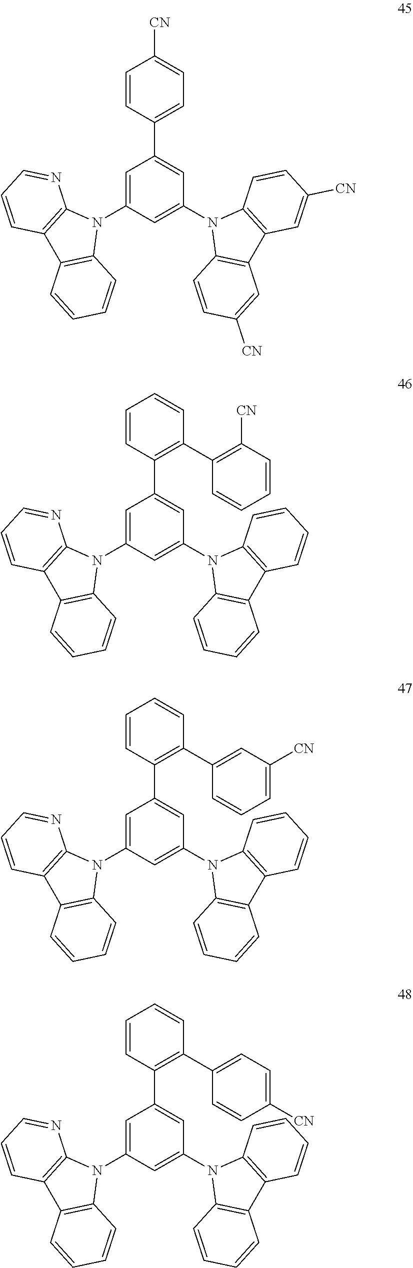 [SMILES: N#Cc1ccc(-c2cc(-n3c4ccc(C#N)cc4c4cc(C#N)ccc43)cc(-n3c4ccccc4c4cccnc43)c2)cc1 ; N#Cc1ccccc1-c1ccccc1-c1cc(-n2c3ccccc3c3ccccc32)cc(-n2c3ccccc3c3cccnc32)c1 ; N#Cc1cccc(-c2ccccc2-c2cc(-n3c4ccccc4c4ccccc43)cc(-n3c4ccccc4c4cccnc43)c2)c1 ; N#Cc1ccc(-c2ccccc2-c2cc(-n3c4ccccc4c4ccccc43)cc(-n3c4ccccc4c4cccnc43)c2)cc1]

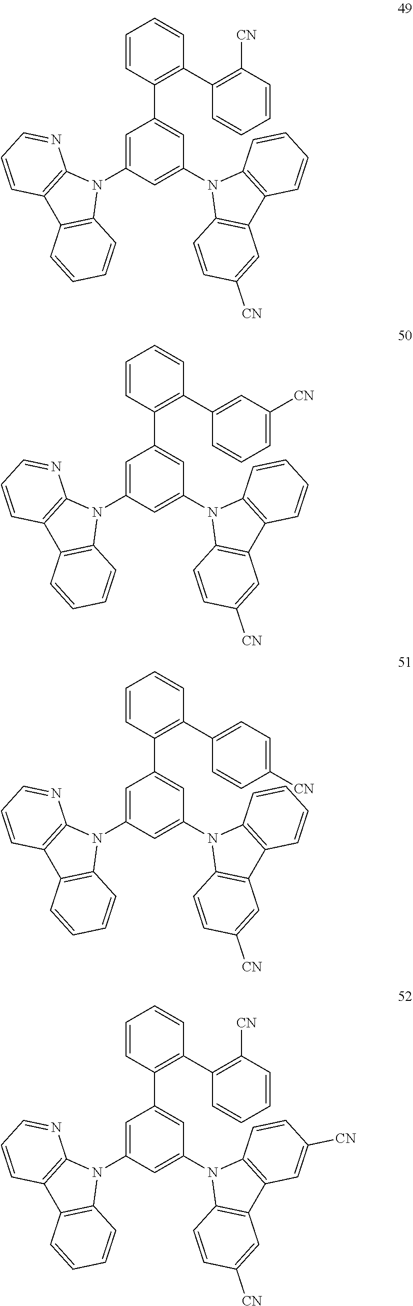 [SMILES: N#Cc1ccc2c(c1)c1ccccc1n2-c1cc(-c2ccccc2-c2ccccc2C#N)cc(-n2c3ccccc3c3cccnc32)c1 ; N#Cc1cccc(-c2ccccc2-c2cc(-n3c4ccccc4c4cc(C#N)ccc43)cc(-n3c4ccccc4c4cccnc43)c2)c1 ; N#Cc1ccc(-c2ccccc2-c2cc(-n3c4ccccc4c4cc(C#N)ccc43)cc(-n3c4ccccc4c4cccnc43)c2)cc1 ; N#Cc1ccc2c(c1)c1cc(C#N)ccc1n2-c1cc(-c2ccccc2-c2ccccc2C#N)cc(-n2c3ccccc3c3cccnc32)c1]

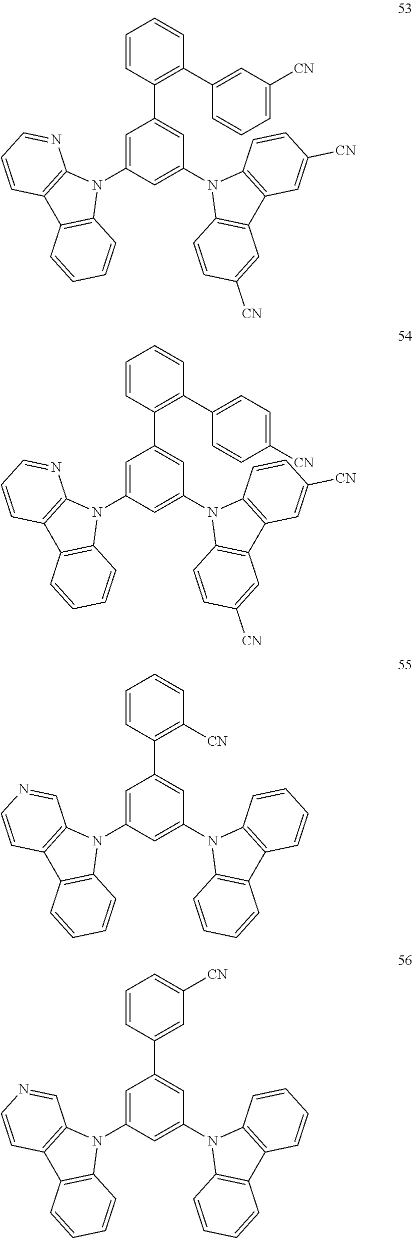

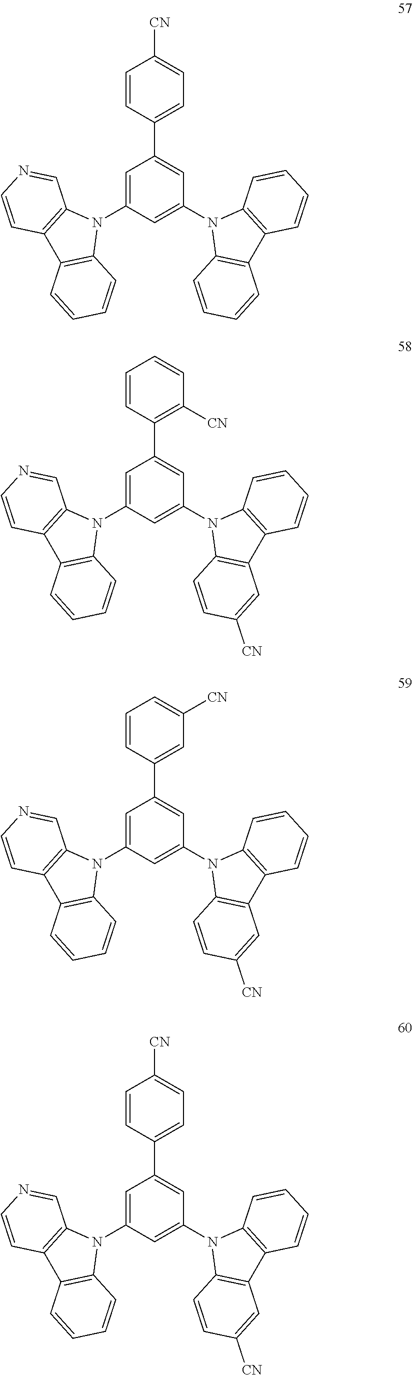 [SMILES: N#Cc1ccc(-c2cc(-n3c4ccccc4c4ccccc43)cc(-n3c4ccccc4c4ccncc43)c2)cc1 ; N#Cc1ccc2c(c1)c1ccccc1n2-c1cc(-c2ccccc2C#N)cc(-n2c3ccccc3c3ccncc32)c1 ; N#Cc1cccc(-c2cc(-n3c4ccccc4c4cc(C#N)ccc43)cc(-n3c4ccccc4c4ccncc43)c2)c1 ; N#Cc1ccc(-c2cc(-n3c4ccccc4c4cc(C#N)ccc43)cc(-n3c4ccccc4c4ccncc43)c2)cc1]

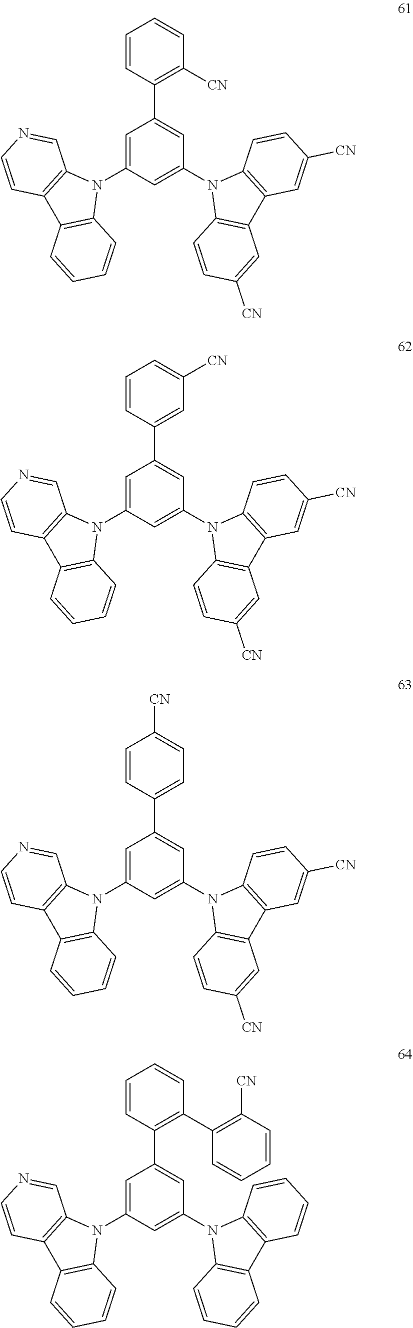 [SMILES: N#Cc1ccc2c(c1)c1cc(C#N)ccc1n2-c1cc(-c2ccccc2C#N)cc(-n2c3ccccc3c3ccncc32)c1 ; N#Cc1cccc(-c2cc(-n3c4ccc(C#N)cc4c4cc(C#N)ccc43)cc(-n3c4ccccc4c4ccncc43)c2)c1 ; N#Cc1ccc(-c2cc(-n3c4ccc(C#N)cc4c4cc(C#N)ccc43)cc(-n3c4ccccc4c4ccncc43)c2)cc1 ; N#Cc1ccccc1-c1ccccc1-c1cc(-n2c3ccccc3c3ccccc32)cc(-n2c3ccccc3c3ccncc32)c1]

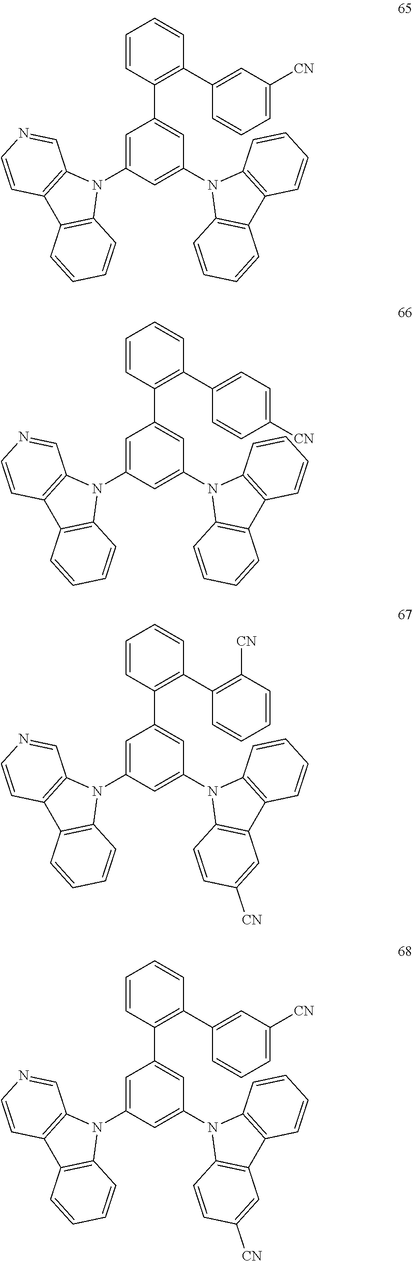 [SMILES: N#Cc1cccc(-c2ccccc2-c2cc(-n3c4ccccc4c4ccccc43)cc(-n3c4ccccc4c4ccncc43)c2)c1 ; N#Cc1ccc(-c2ccccc2-c2cc(-n3c4ccccc4c4ccccc43)cc(-n3c4ccccc4c4ccncc43)c2)cc1 ; N#Cc1ccc2c(c1)c1ccccc1n2-c1cc(-c2ccccc2-c2ccccc2C#N)cc(-n2c3ccccc3c3ccncc32)c1 ; N#Cc1cccc(-c2ccccc2-c2cc(-n3c4ccccc4c4cc(C#N)ccc43)cc(-n3c4ccccc4c4ccncc43)c2)c1]

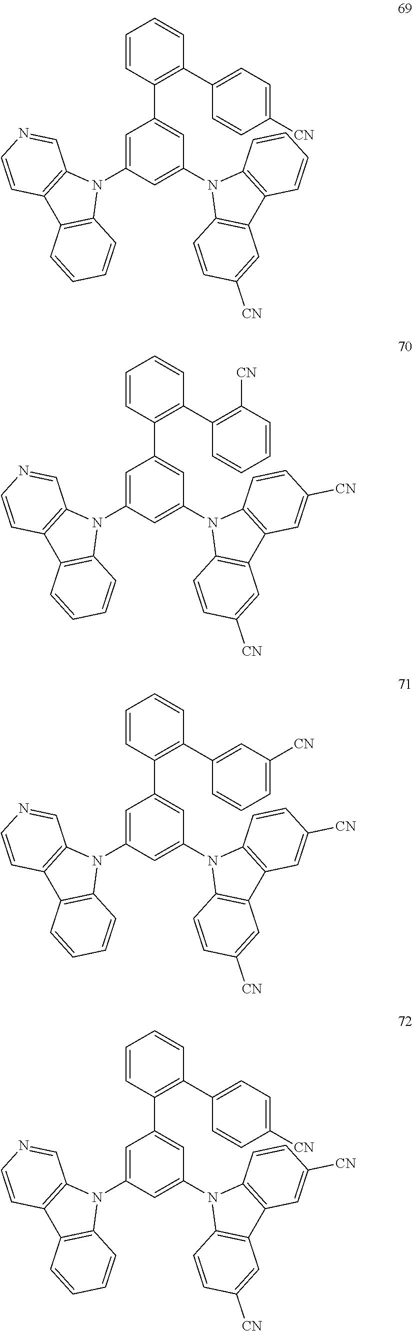 [SMILES: N#Cc1ccc(-c2ccccc2-c2cc(-n3c4ccccc4c4cc(C#N)ccc43)cc(-n3c4ccccc4c4ccncc43)c2)cc1 ; N#Cc1ccc2c(c1)c1cc(C#N)ccc1n2-c1cc(-c2ccccc2-c2ccccc2C#N)cc(-n2c3ccccc3c3ccncc32)c1 ; N#Cc1cccc(-c2ccccc2-c2cc(-n3c4ccc(C#N)cc4c4cc(C#N)ccc43)cc(-n3c4ccccc4c4ccncc43)c2)c1 ; N#Cc1ccc(-c2ccccc2-c2cc(-n3c4ccc(C#N)cc4c4cc(C#N)ccc43)cc(-n3c4ccccc4c4ccncc43)c2)cc1]

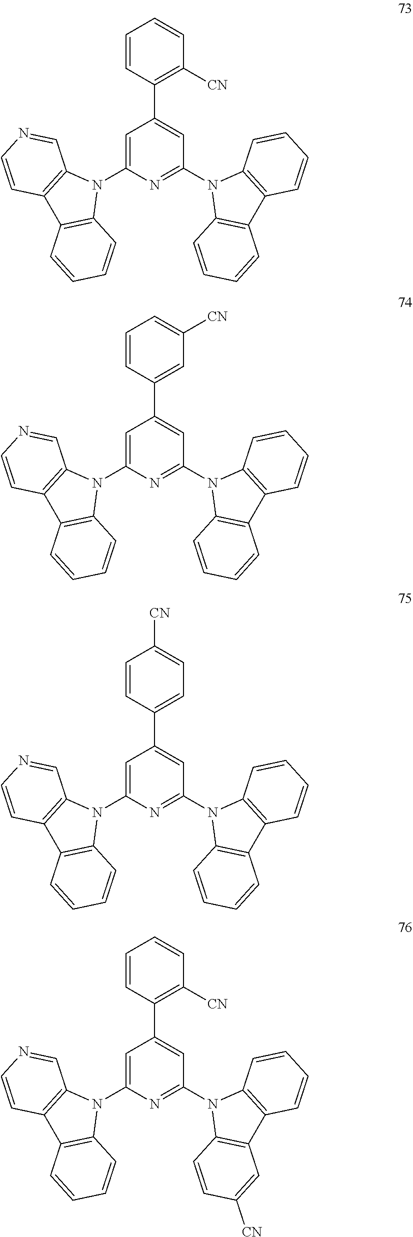

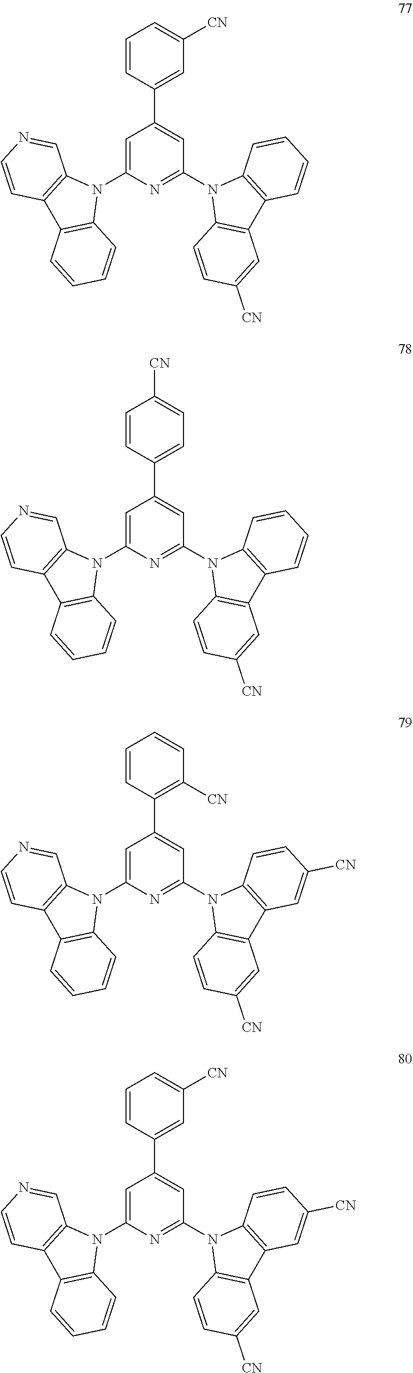 [SMILES: N#Cc1cccc(-c2cc(-n3c4ccccc4c4cc(C#N)ccc43)nc(-n3c4ccccc4c4ccncc43)c2)c1 ; N#Cc1ccc(-c2cc(-n3c4ccccc4c4cc(C#N)ccc43)nc(-n3c4ccccc4c4ccncc43)c2)cc1 ; N#Cc1ccc2c(c1)c1cc(C#N)ccc1n2-c1cc(-c2ccccc2C#N)cc(-n2c3ccccc3c3ccncc32)n1 ; N#Cc1cccc(-c2cc(-n3c4ccc(C#N)cc4c4cc(C#N)ccc43)nc(-n3c4ccccc4c4ccncc43)c2)c1]

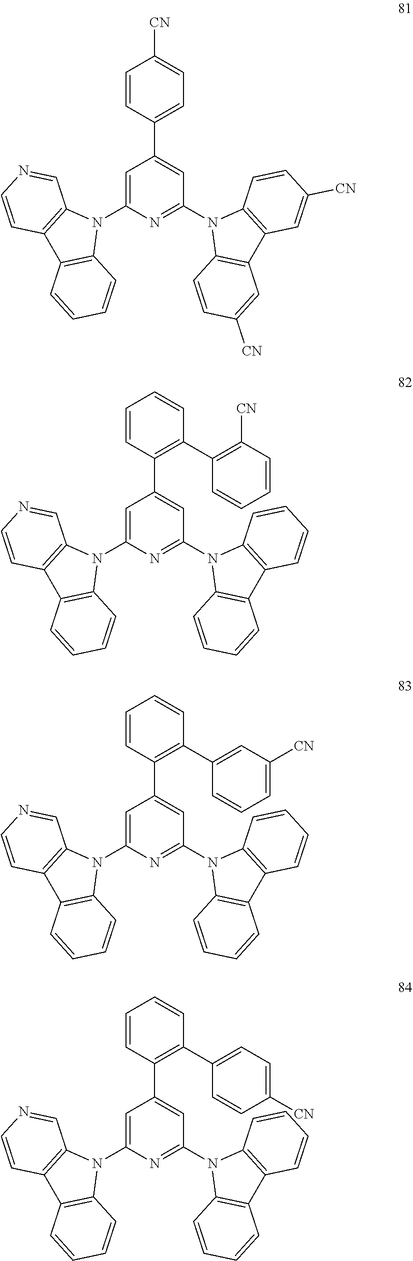 [SMILES: N#Cc1ccc(-c2cc(-n3c4ccc(C#N)cc4c4cc(C#N)ccc43)nc(-n3c4ccccc4c4ccncc43)c2)cc1 ; N#Cc1ccccc1-c1ccccc1-c1cc(-n2c3ccccc3c3ccccc32)nc(-n2c3ccccc3c3ccncc32)c1 ; N#Cc1cccc(-c2ccccc2-c2cc(-n3c4ccccc4c4ccccc43)nc(-n3c4ccccc4c4ccncc43)c2)c1 ; N#Cc1ccc(-c2ccccc2-c2cc(-n3c4ccccc4c4ccccc43)nc(-n3c4ccccc4c4ccncc43)c2)cc1]

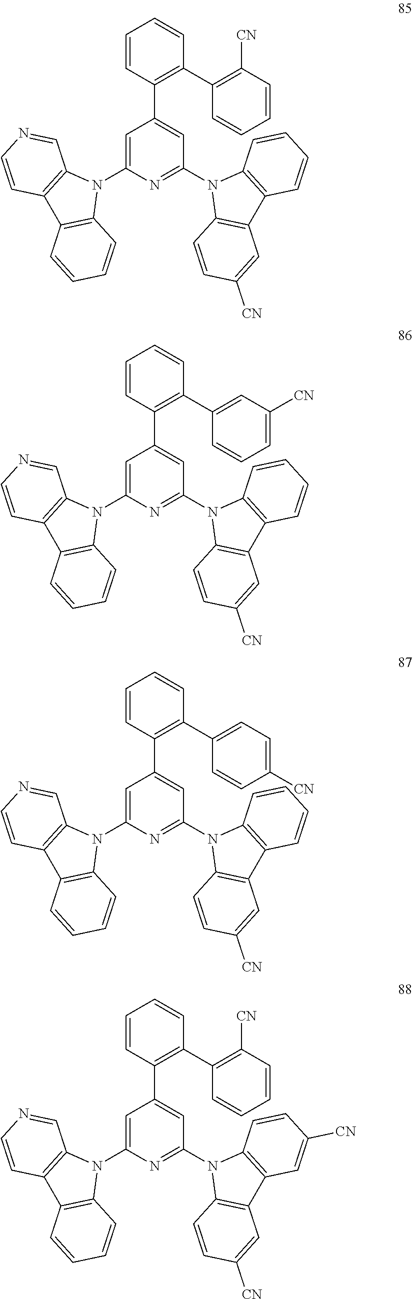 [SMILES: N#Cc1ccc2c(c1)c1ccccc1n2-c1cc(-c2ccccc2-c2ccccc2C#N)cc(-n2c3ccccc3c3ccncc32)n1 ; N#Cc1cccc(-c2ccccc2-c2cc(-n3c4ccccc4c4cc(C#N)ccc43)nc(-n3c4ccccc4c4ccncc43)c2)c1 ; N#Cc1ccc(-c2ccccc2-c2cc(-n3c4ccccc4c4cc(C#N)ccc43)nc(-n3c4ccccc4c4ccncc43)c2)cc1 ; N#Cc1ccc2c(c1)c1cc(C#N)ccc1n2-c1cc(-c2ccccc2-c2ccccc2C#N)cc(-n2c3ccccc3c3ccncc32)n1]

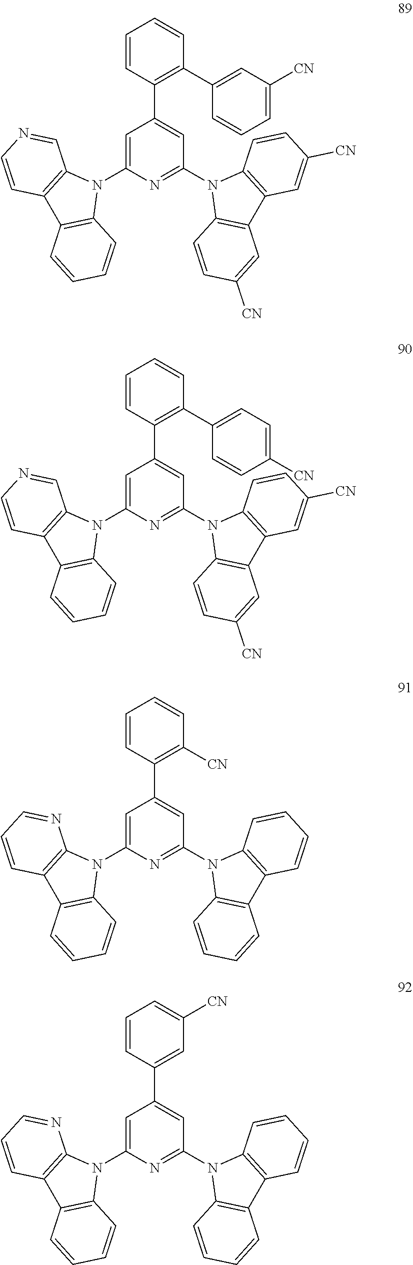 [SMILES: N#Cc1cccc(-c2ccccc2-c2cc(-n3c4ccc(C#N)cc4c4cc(C#N)ccc43)nc(-n3c4ccccc4c4ccncc43)c2)c1 ; N#Cc1ccc(-c2ccccc2-c2cc(-n3c4ccc(C#N)cc4c4cc(C#N)ccc43)nc(-n3c4ccccc4c4ccncc43)c2)cc1 ; N#Cc1ccccc1-c1cc(-n2c3ccccc3c3ccccc32)nc(-n2c3ccccc3c3cccnc32)c1 ; N#Cc1cccc(-c2cc(-n3c4ccccc4c4ccccc43)nc(-n3c4ccccc4c4cccnc43)c2)c1]

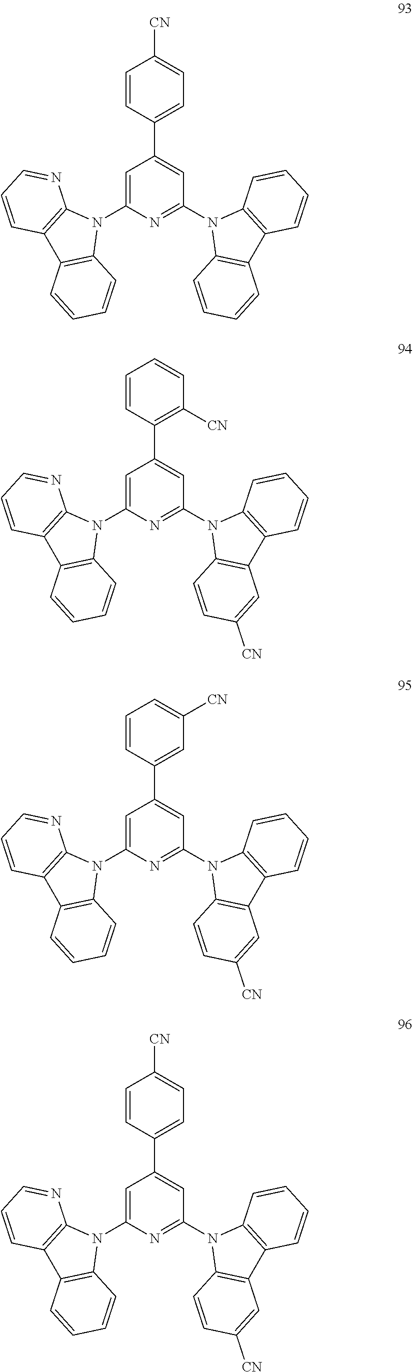 [SMILES: N#Cc1ccc(-c2cc(-n3c4ccccc4c4ccccc43)nc(-n3c4ccccc4c4cccnc43)c2)cc1 ; N#Cc1ccc2c(c1)c1ccccc1n2-c1cc(-c2ccccc2C#N)cc(-n2c3ccccc3c3cccnc32)n1 ; N#Cc1cccc(-c2cc(-n3c4ccccc4c4cc(C#N)ccc43)nc(-n3c4ccccc4c4cccnc43)c2)c1 ; N#Cc1ccc(-c2cc(-n3c4ccccc4c4cc(C#N)ccc43)nc(-n3c4ccccc4c4cccnc43)c2)cc1]

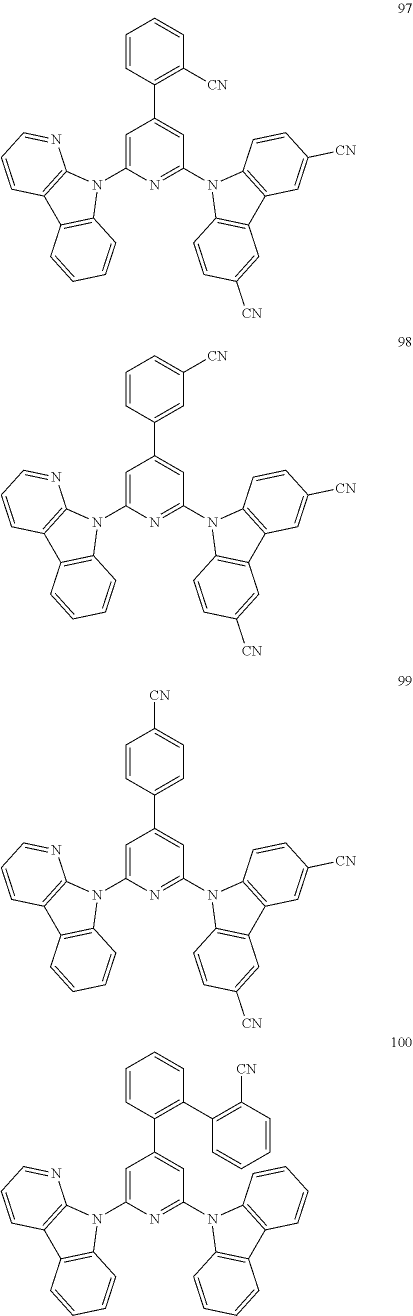

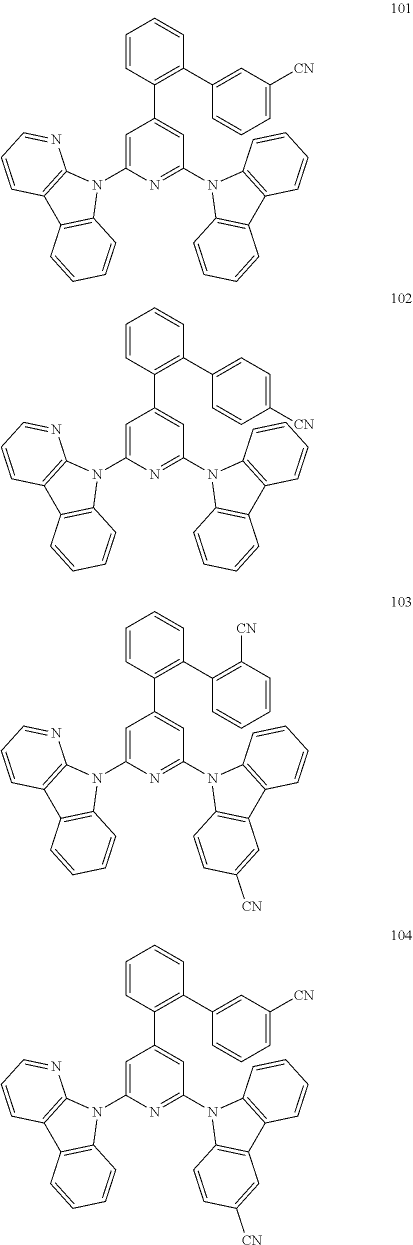 [SMILES: N#Cc1cccc(-c2ccccc2-c2cc(-n3c4ccccc4c4ccccc43)nc(-n3c4ccccc4c4cccnc43)c2)c1 ; N#Cc1ccc(-c2ccccc2-c2cc(-n3c4ccccc4c4ccccc43)nc(-n3c4ccccc4c4cccnc43)c2)cc1 ; N#Cc1ccc2c(c1)c1ccccc1n2-c1cc(-c2ccccc2-c2ccccc2C#N)cc(-n2c3ccccc3c3cccnc32)n1 ; N#Cc1cccc(-c2ccccc2-c2cc(-n3c4ccccc4c4cc(C#N)ccc43)nc(-n3c4ccccc4c4cccnc43)c2)c1]

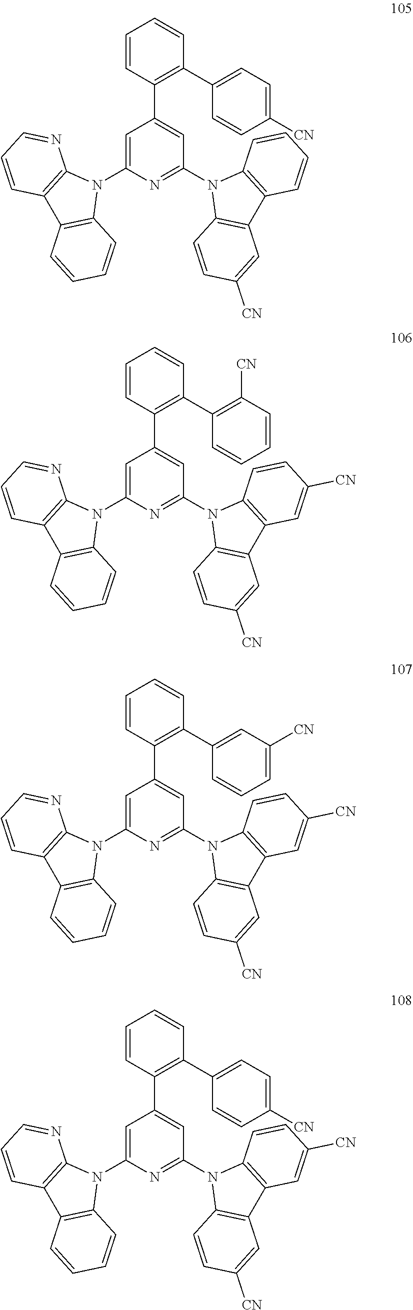 [SMILES: N#Cc1ccc(-c2ccccc2-c2cc(-n3c4ccccc4c4cc(C#N)ccc43)nc(-n3c4ccccc4c4cccnc43)c2)cc1 ; N#Cc1ccc2c(c1)c1cc(C#N)ccc1n2-c1cc(-c2ccccc2-c2ccccc2C#N)cc(-n2c3ccccc3c3cccnc32)n1 ; N#Cc1cccc(-c2ccccc2-c2cc(-n3c4ccc(C#N)cc4c4cc(C#N)ccc43)nc(-n3c4ccccc4c4cccnc43)c2)c1 ; N#Cc1ccc(-c2ccccc2-c2cc(-n3c4ccc(C#N)cc4c4cc(C#N)ccc43)nc(-n3c4ccccc4c4cccnc43)c2)cc1]

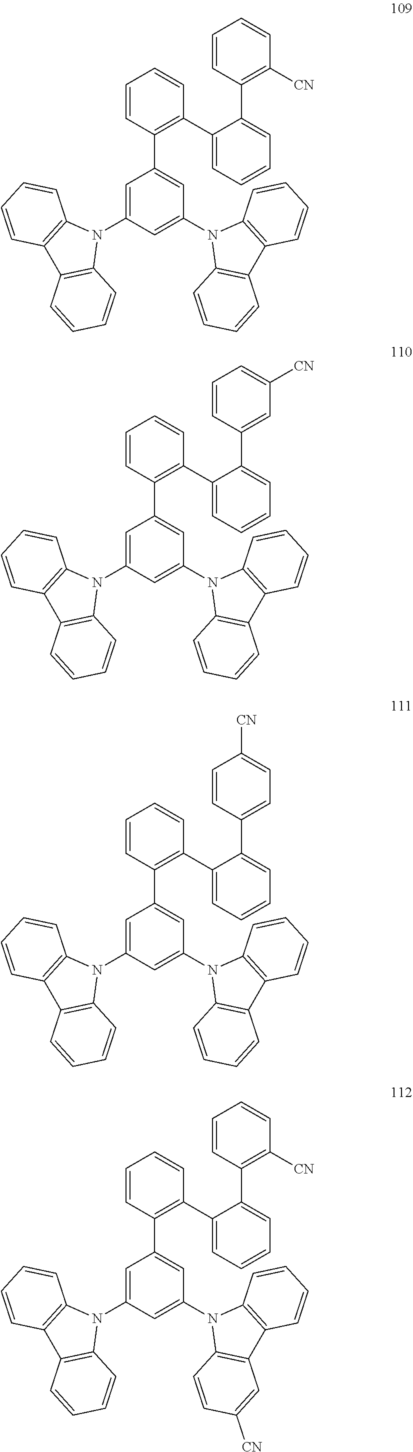

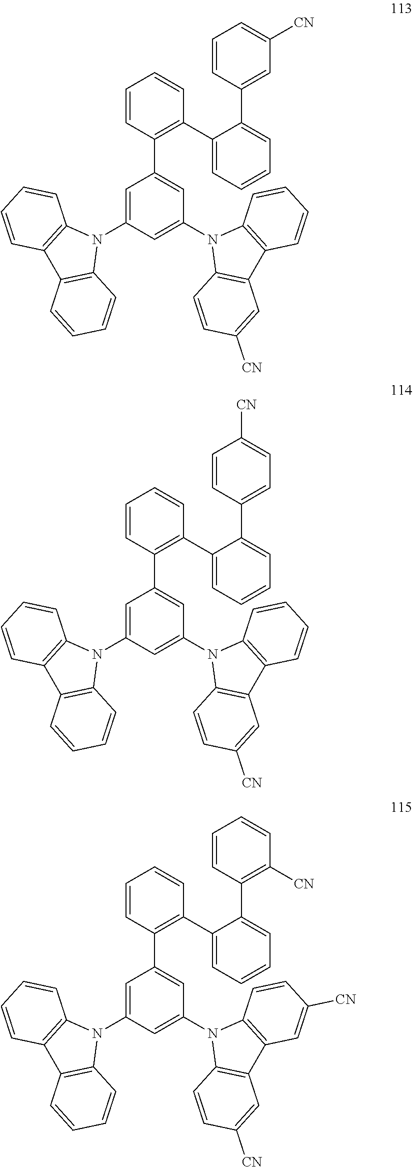

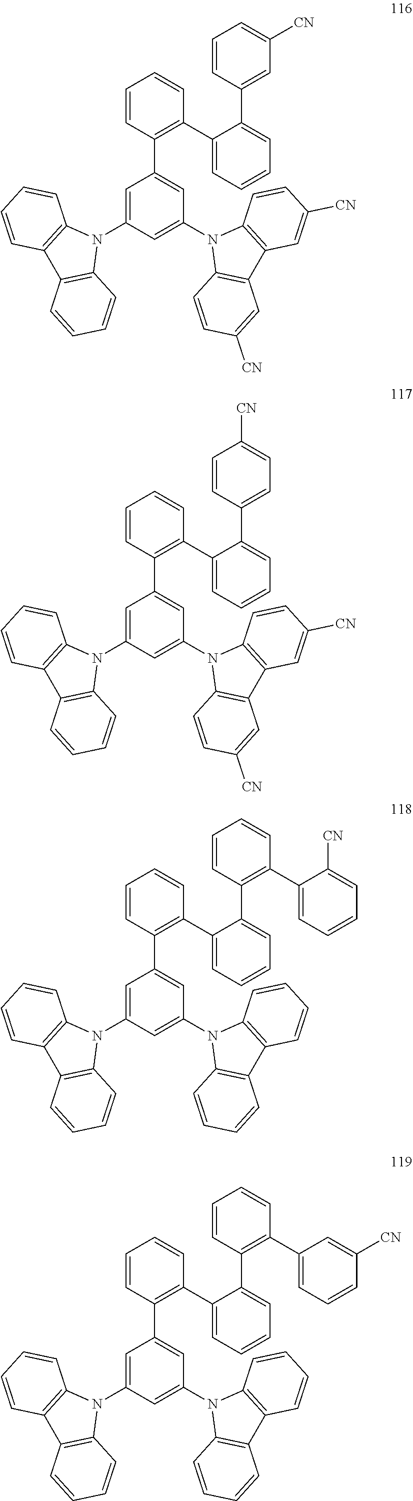

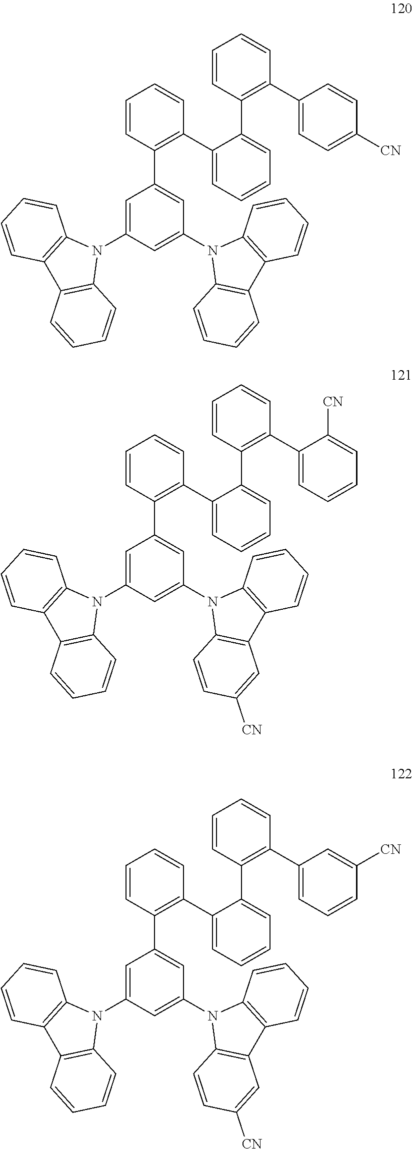

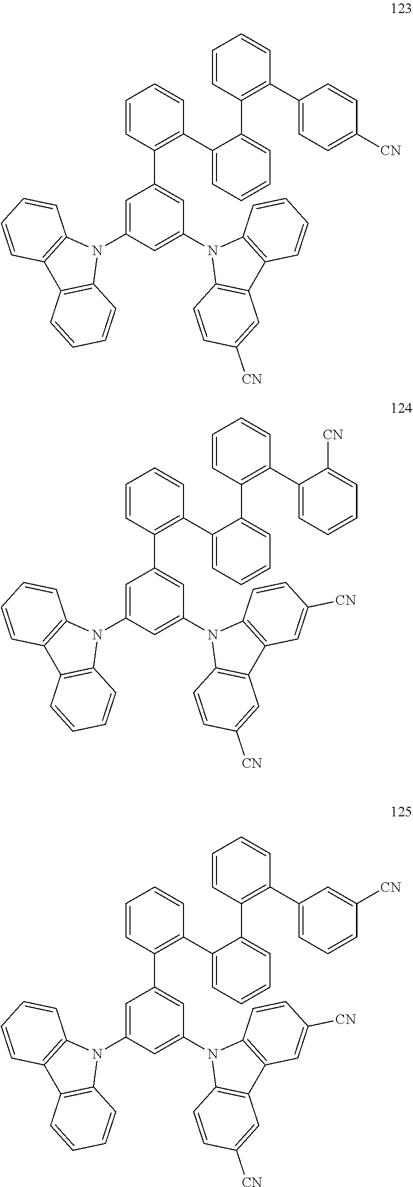

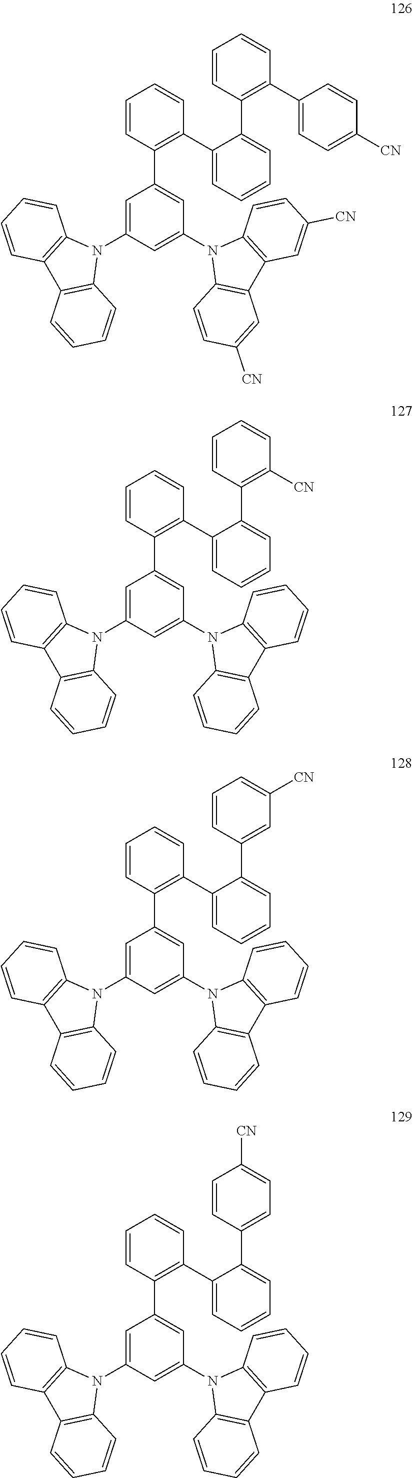

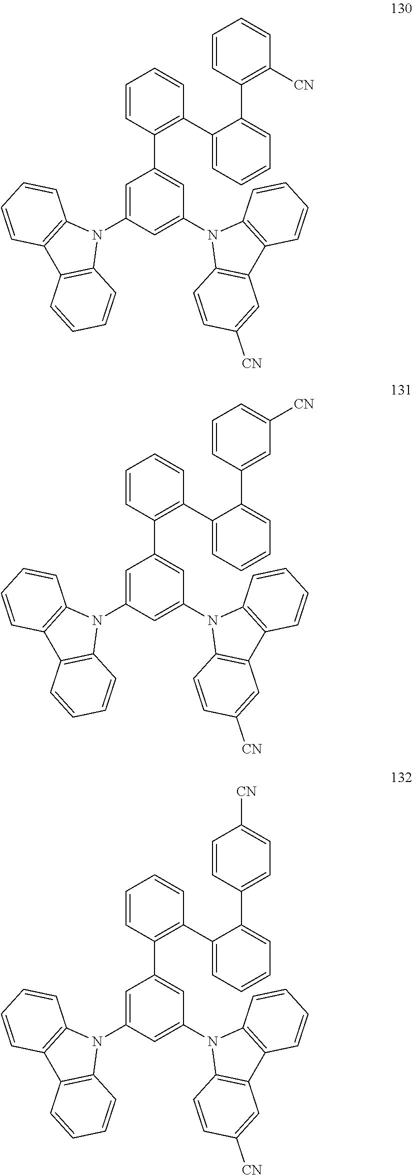

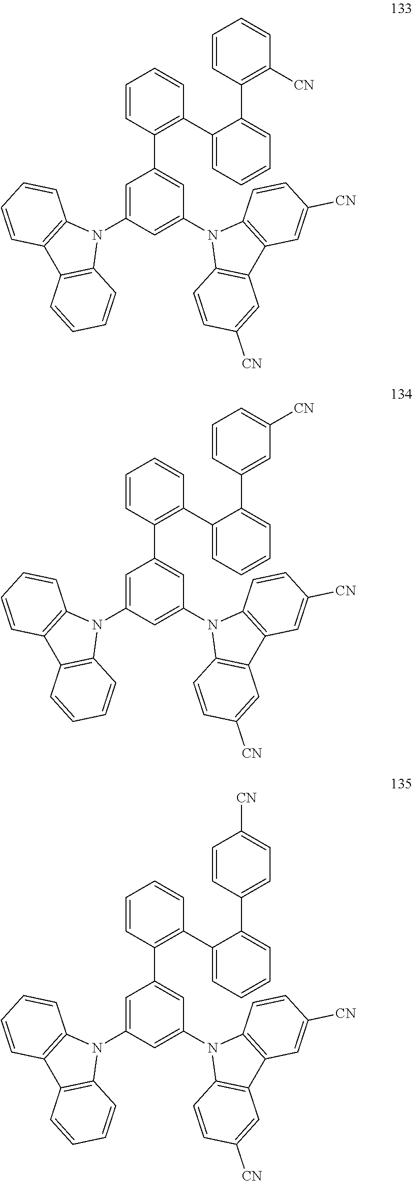

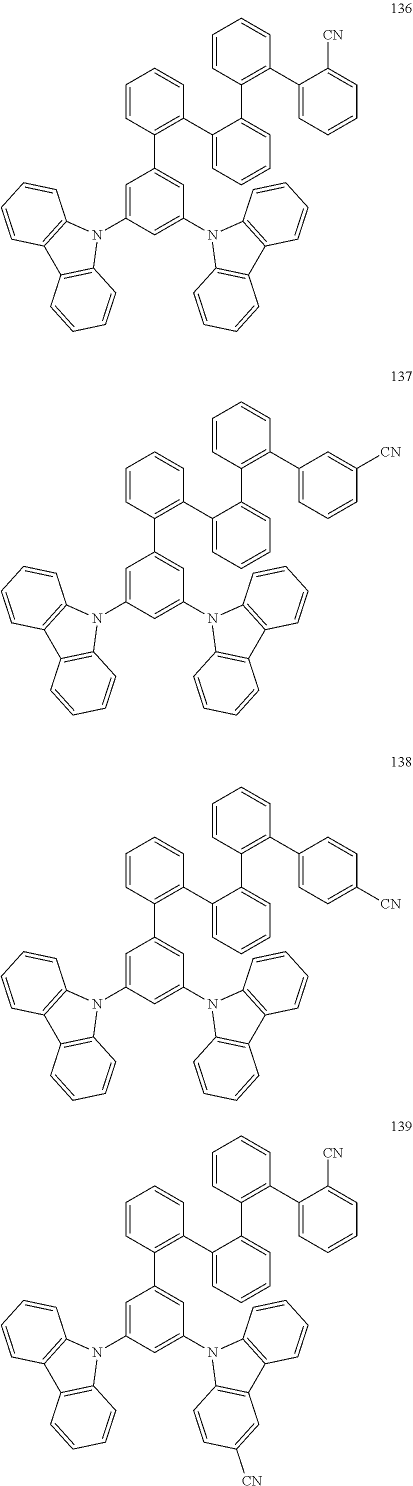

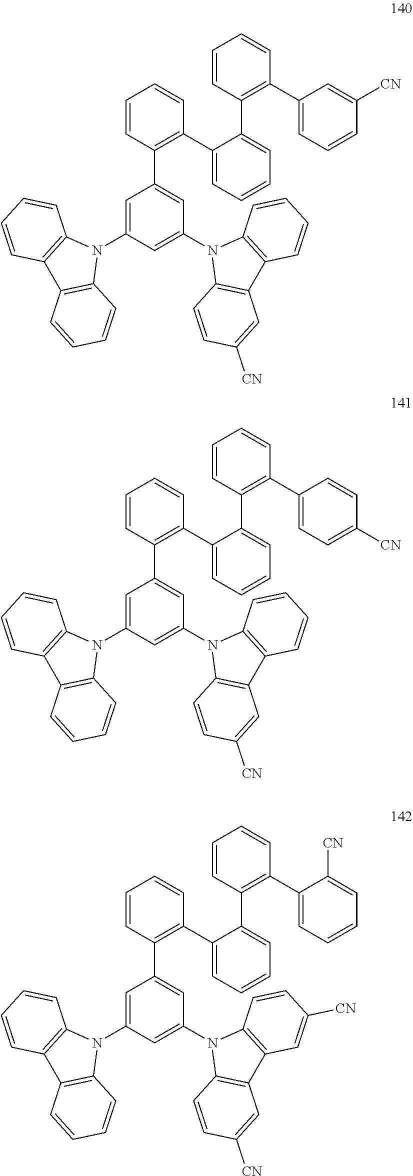 [SMILES: N#Cc1cccc(-c2ccccc2-c2ccccc2-c2ccccc2-c2cc(-n3c4ccccc4c4ccccc43)cc(-n3c4ccccc4c4cc(C#N)ccc43)c2)c1 ; N#Cc1ccc(-c2ccccc2-c2ccccc2-c2ccccc2-c2cc(-n3c4ccccc4c4ccccc43)cc(-n3c4ccccc4c4cc(C#N)ccc43)c2)cc1 ; N#Cc1ccc2c(c1)c1cc(C#N)ccc1n2-c1cc(-c2ccccc2-c2ccccc2-c2ccccc2-c2ccccc2C#N)cc(-n2c3ccccc3c3ccccc32)c1]

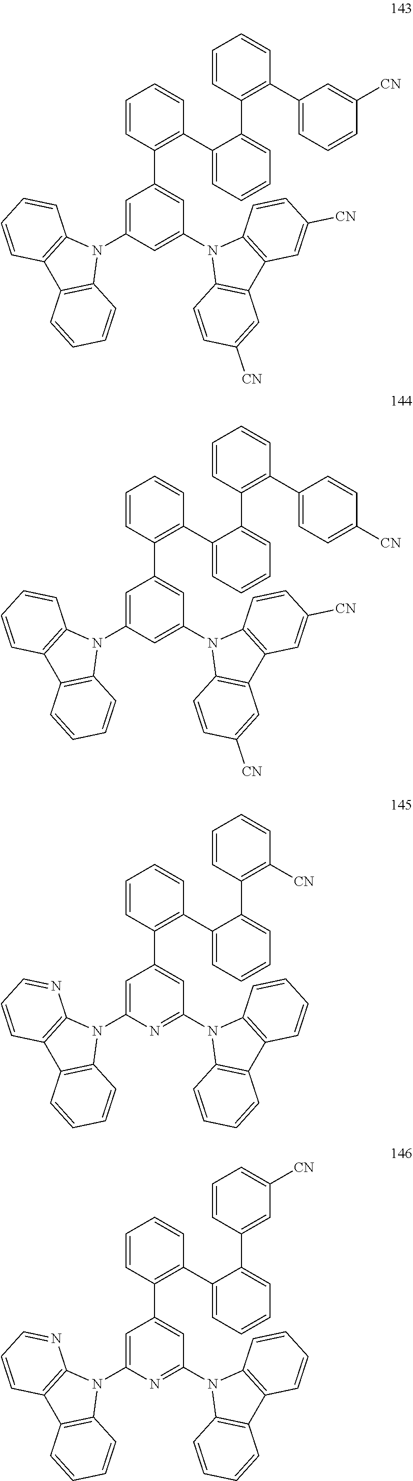

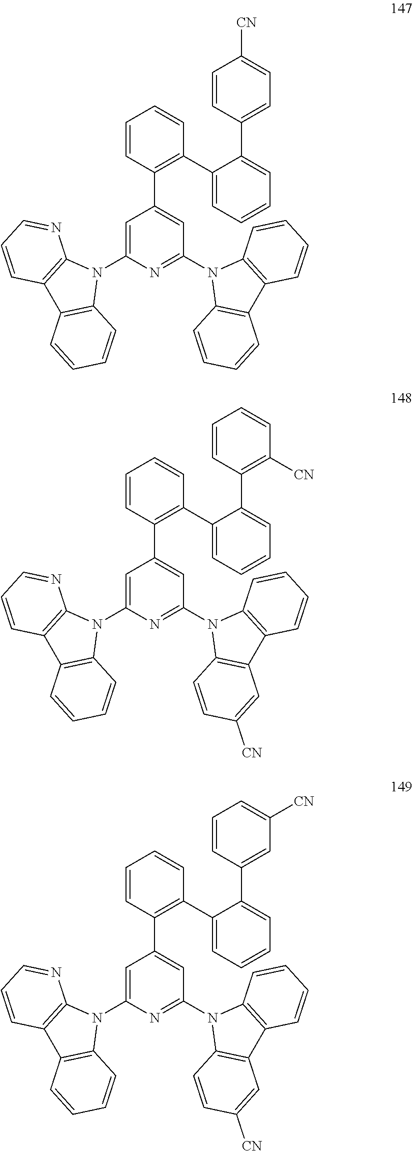

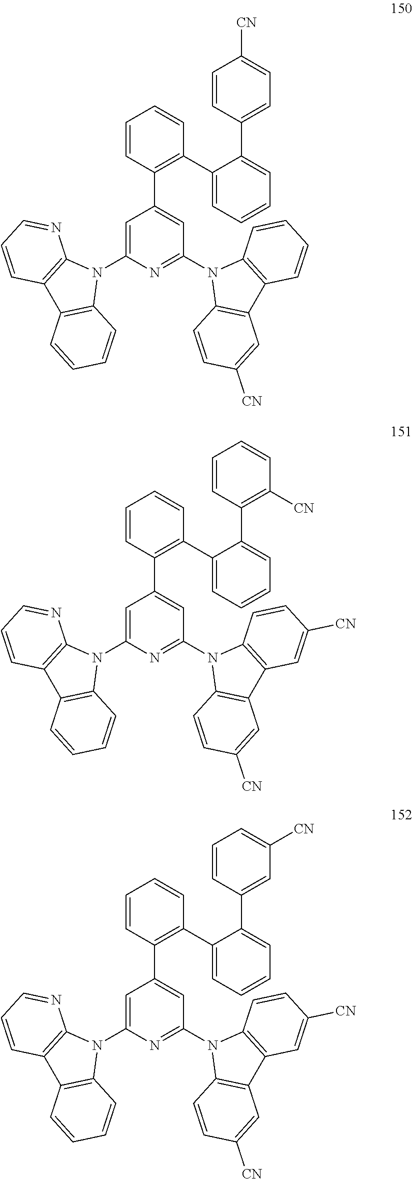

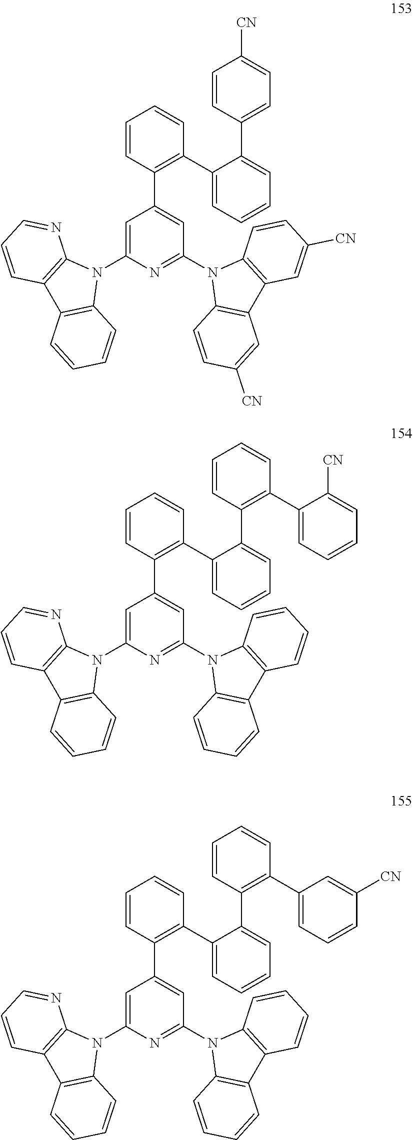

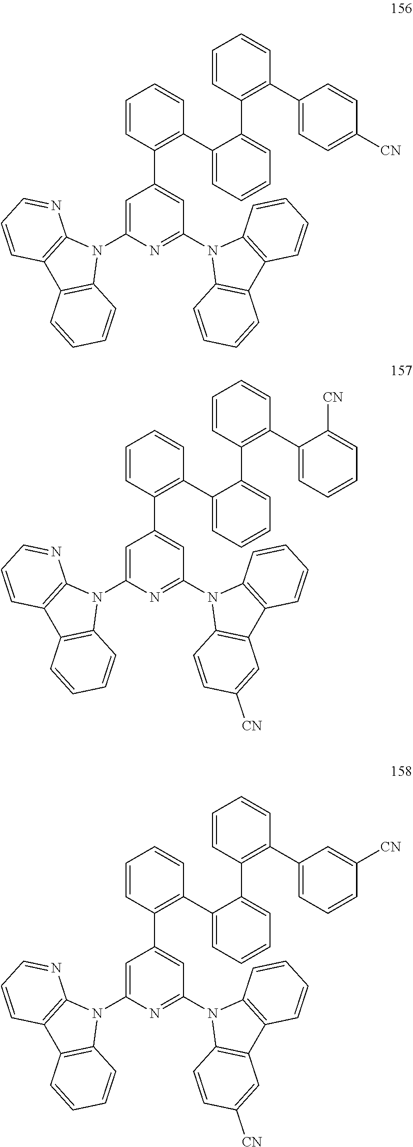

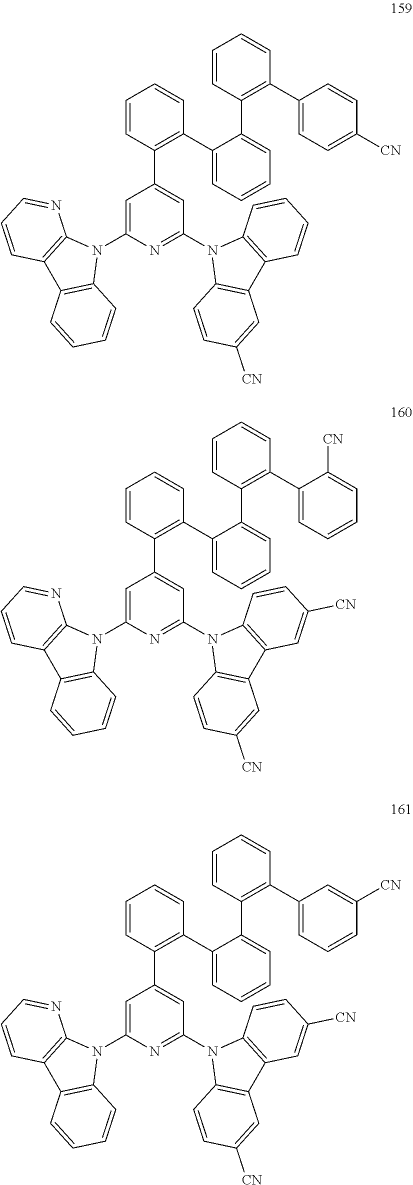

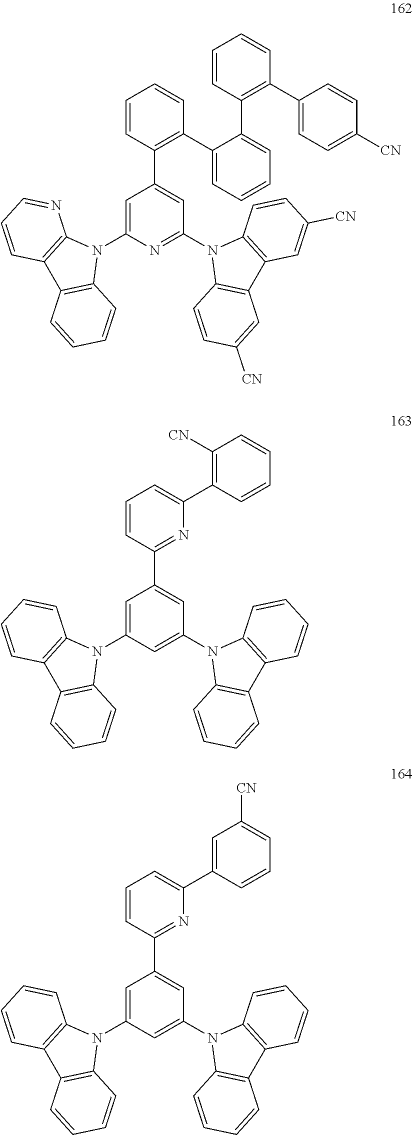

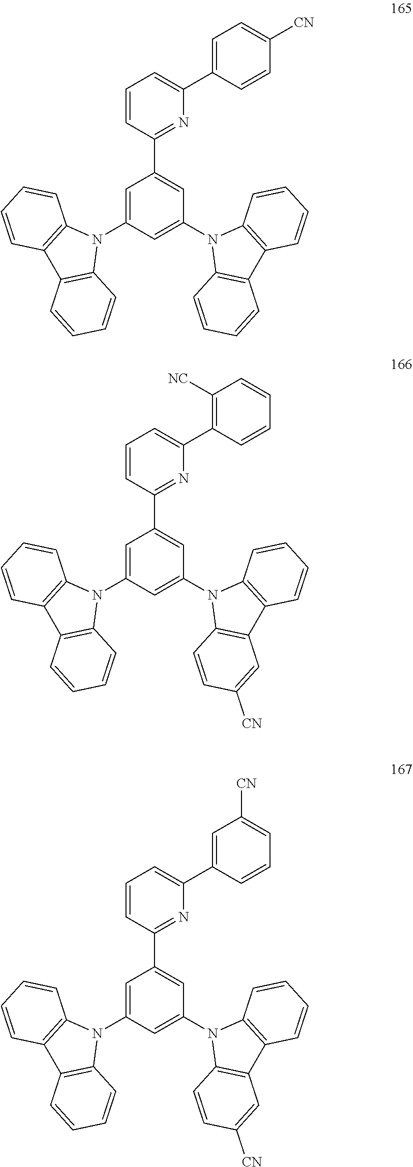

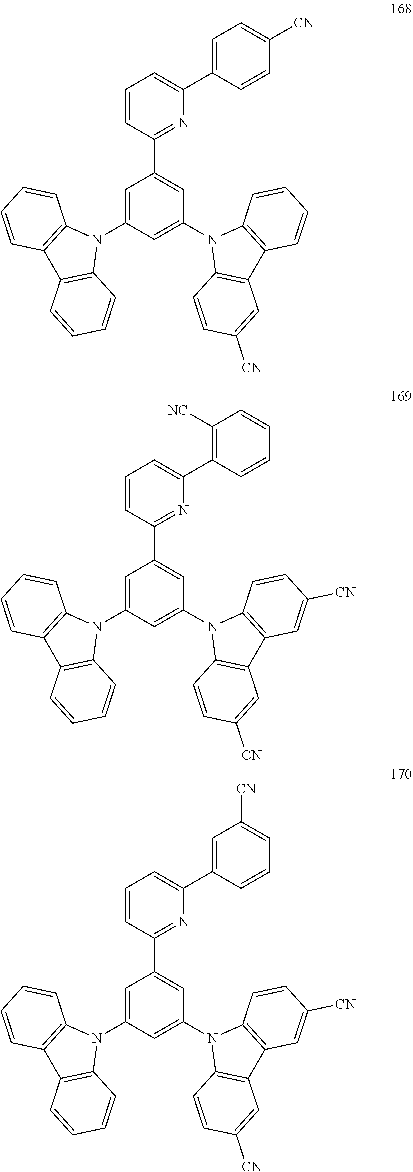

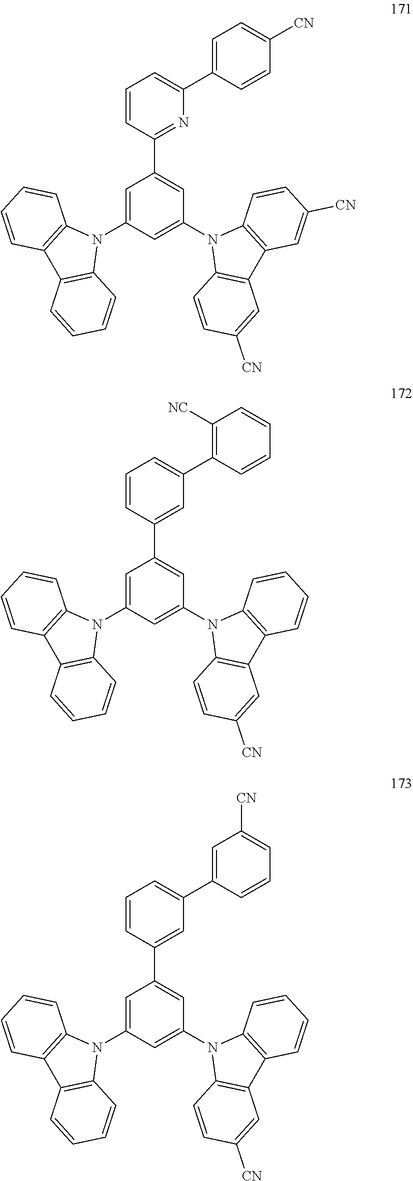

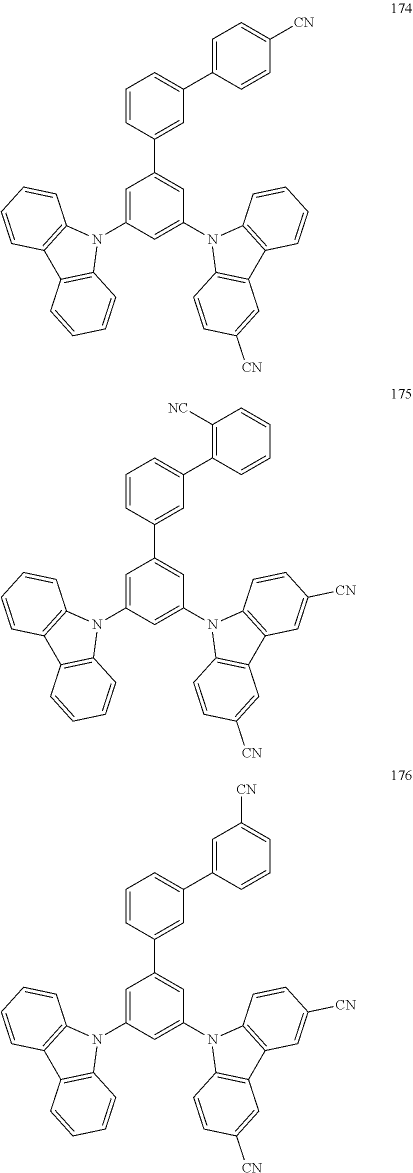

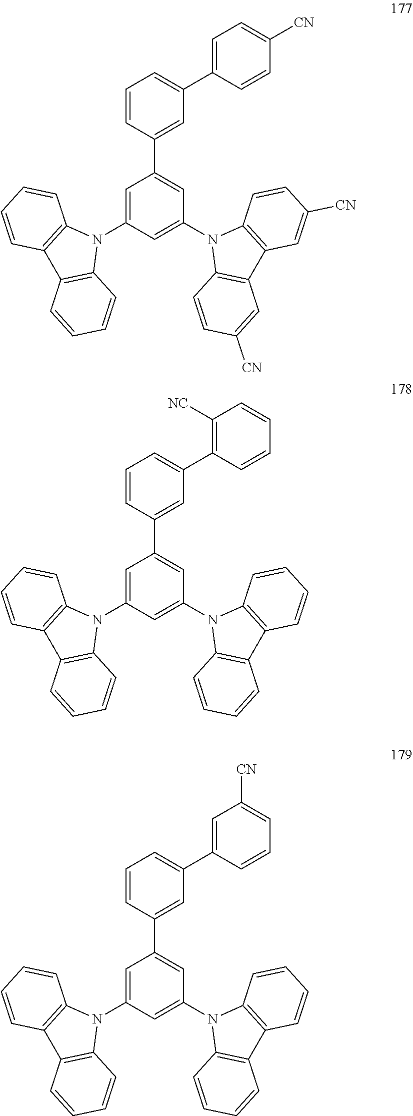

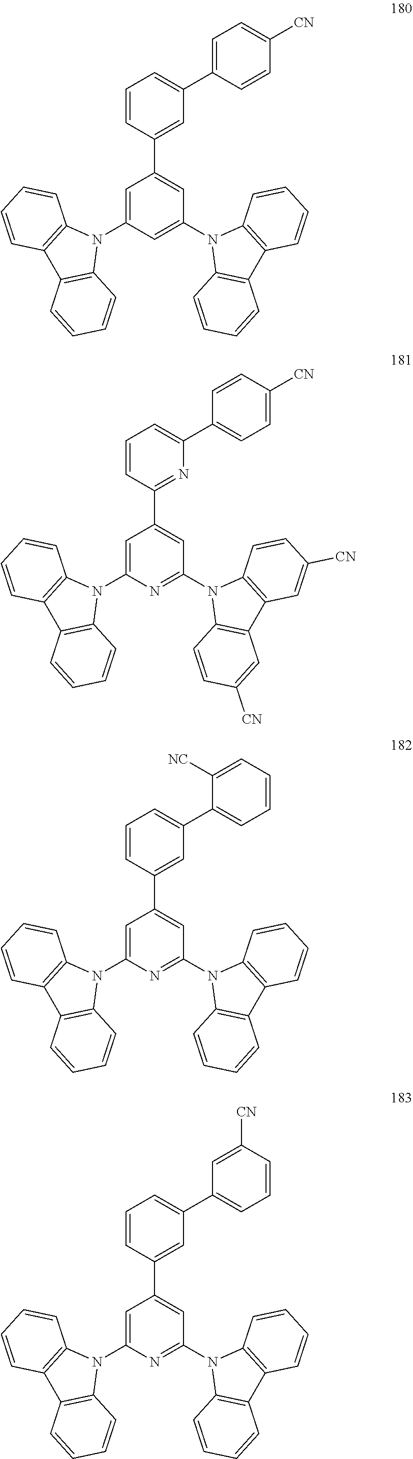

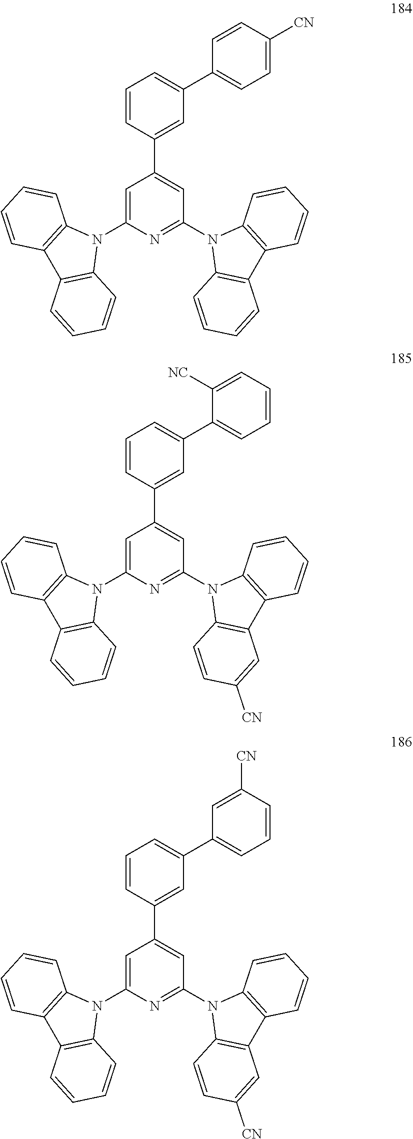

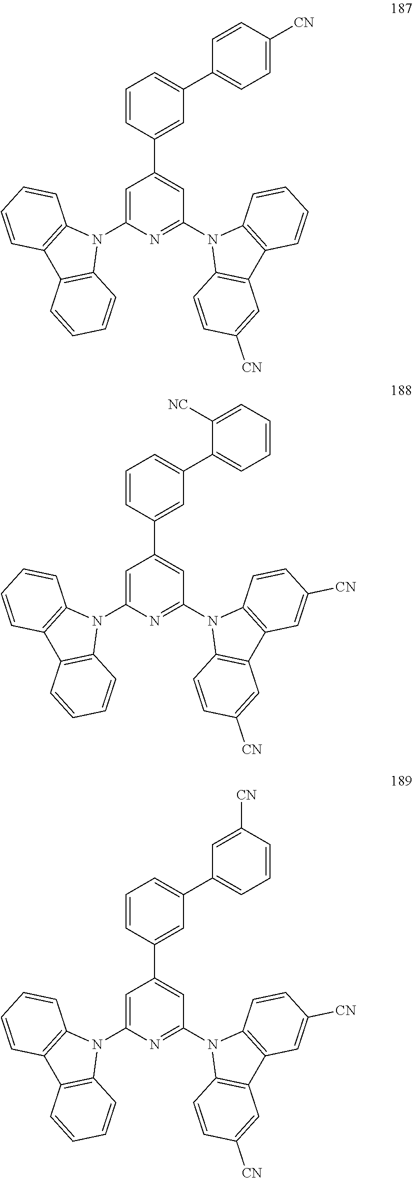

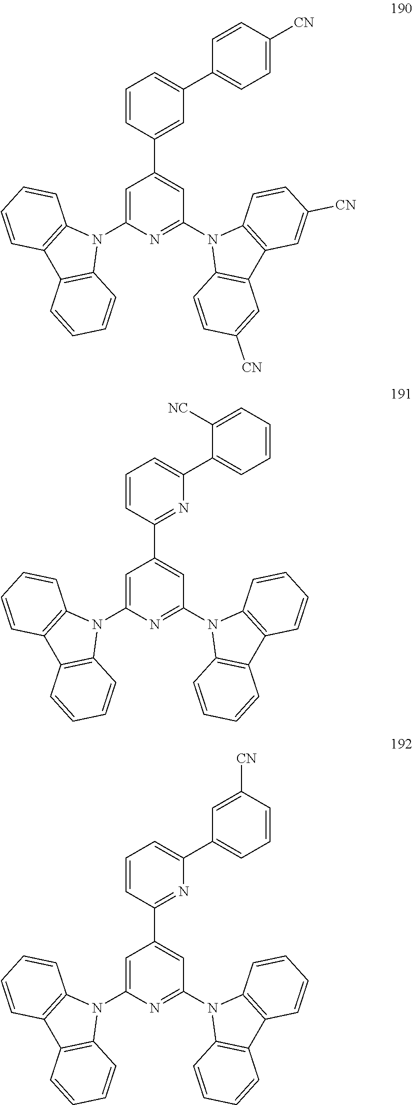

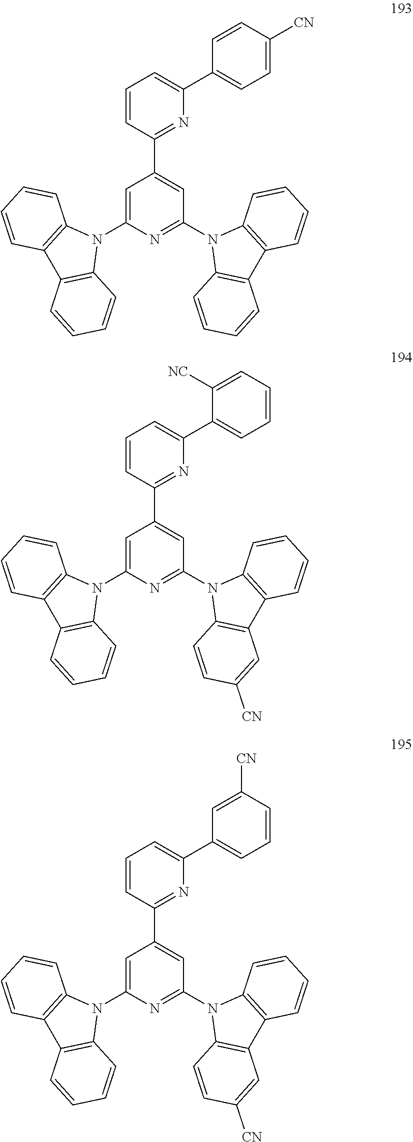

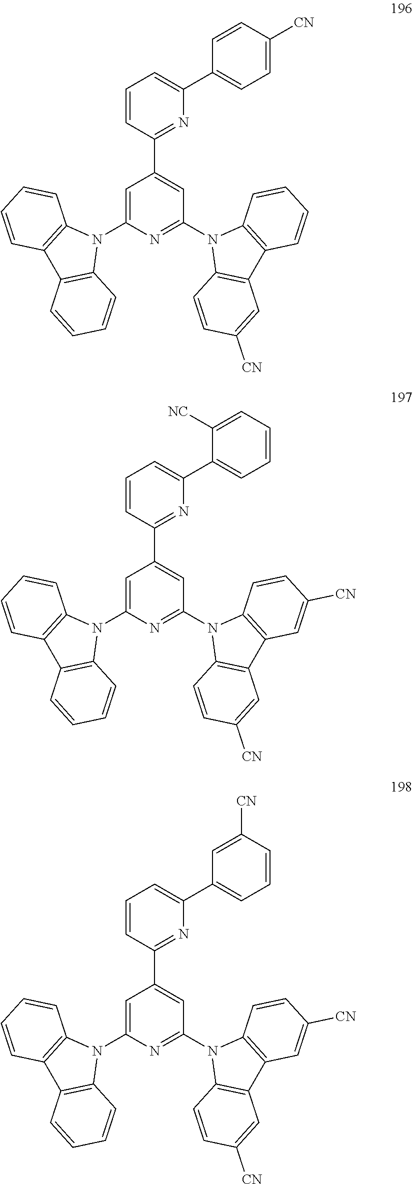

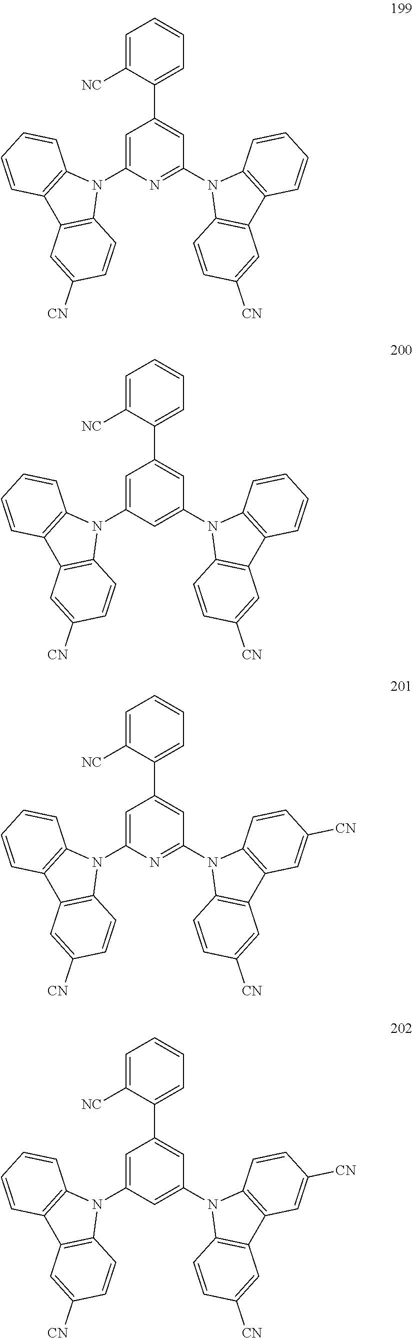

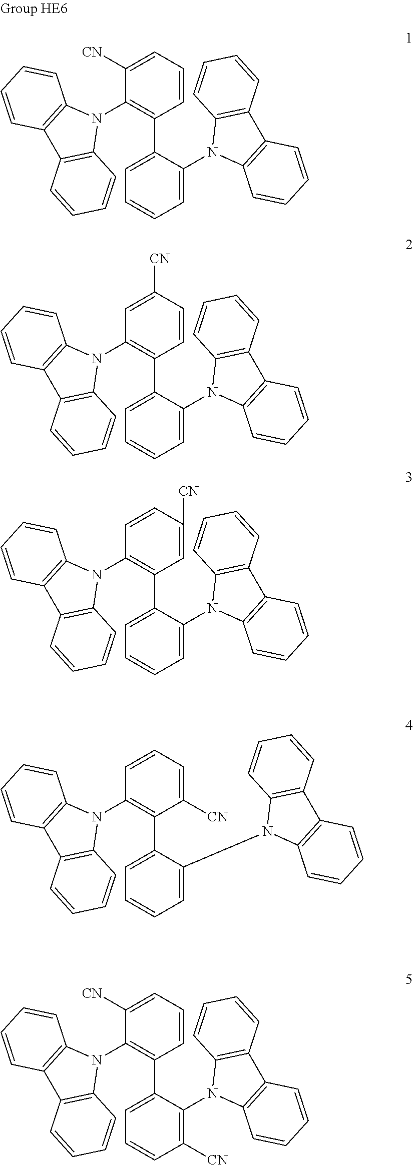

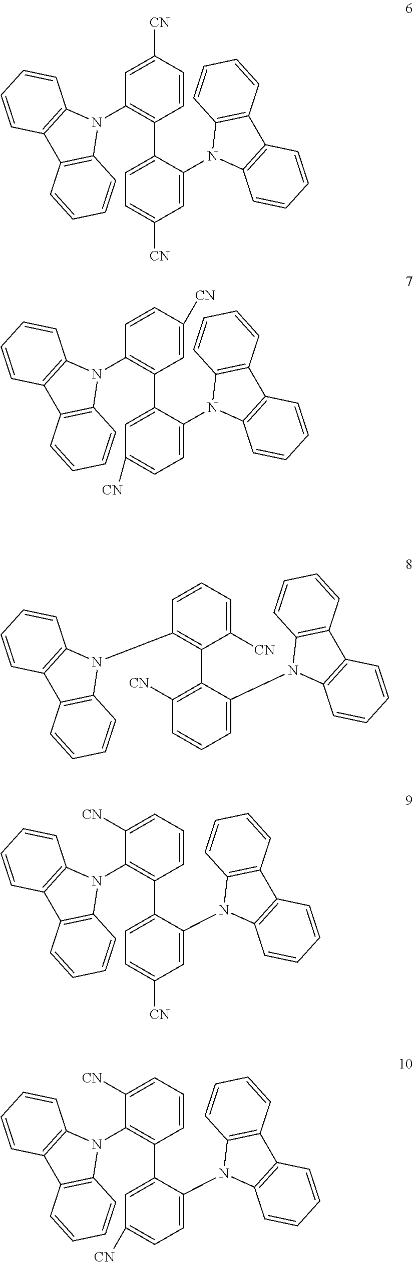

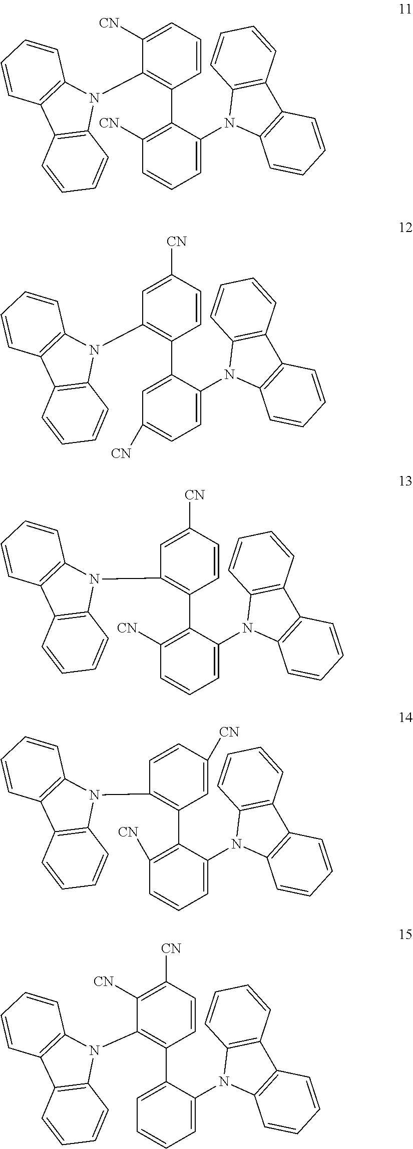

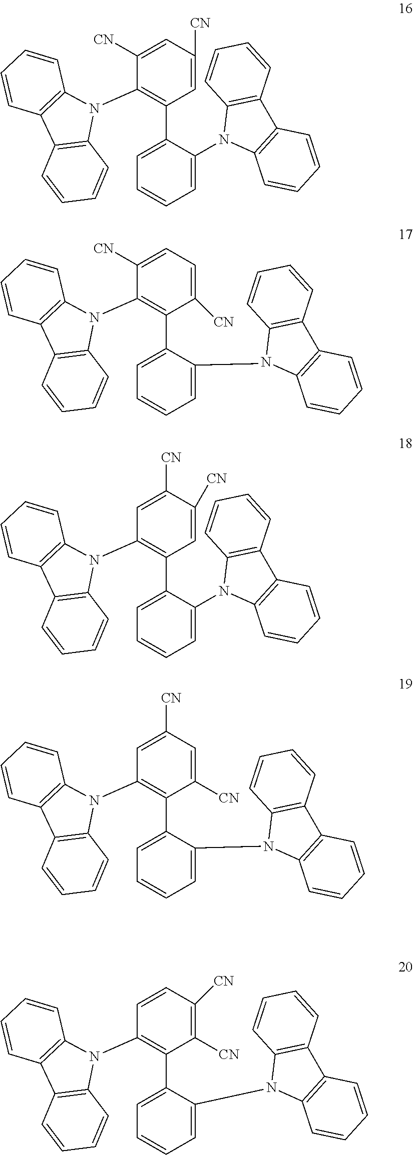

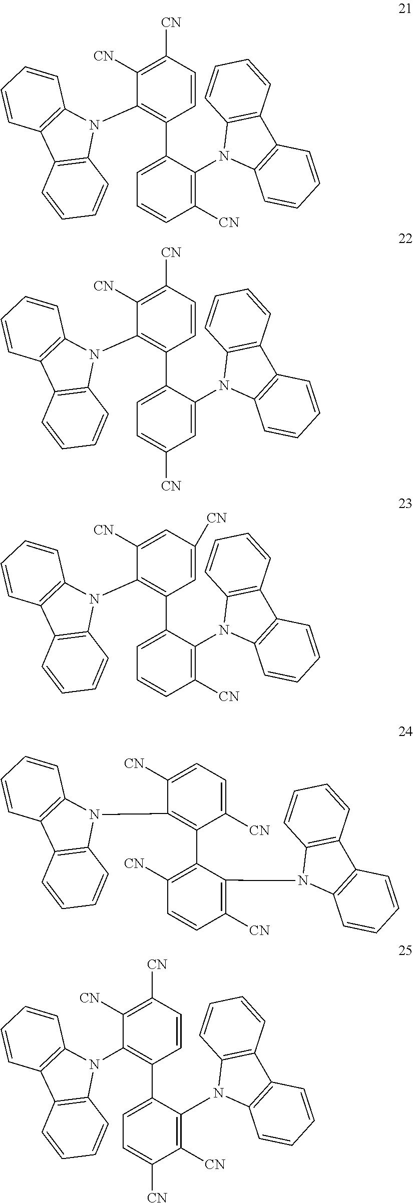

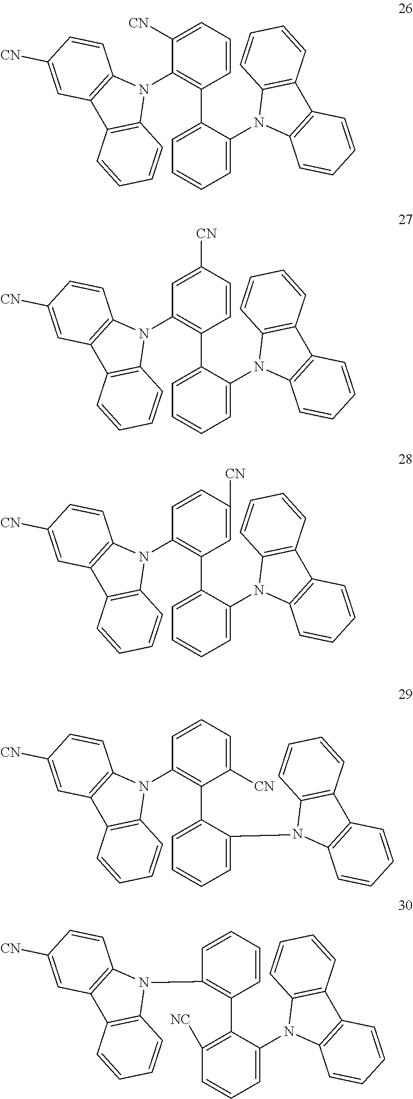

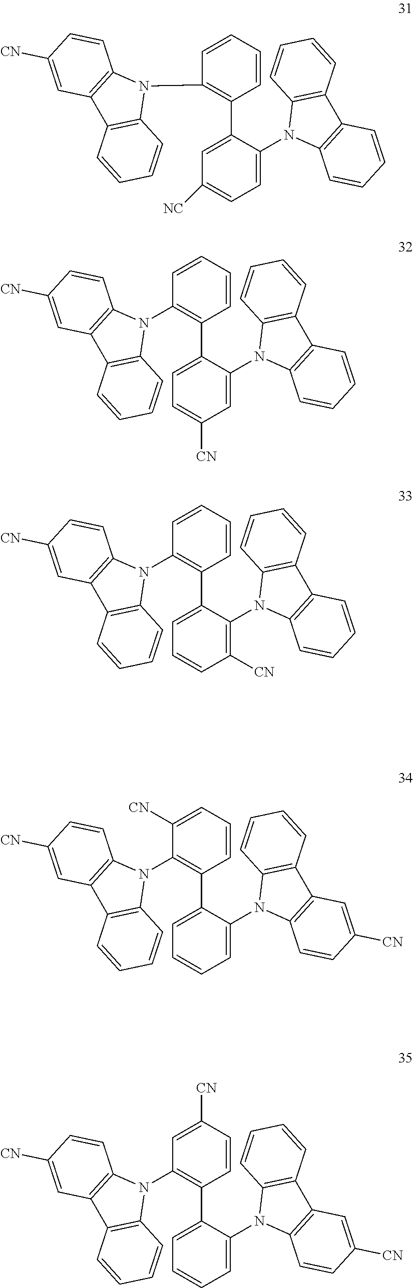

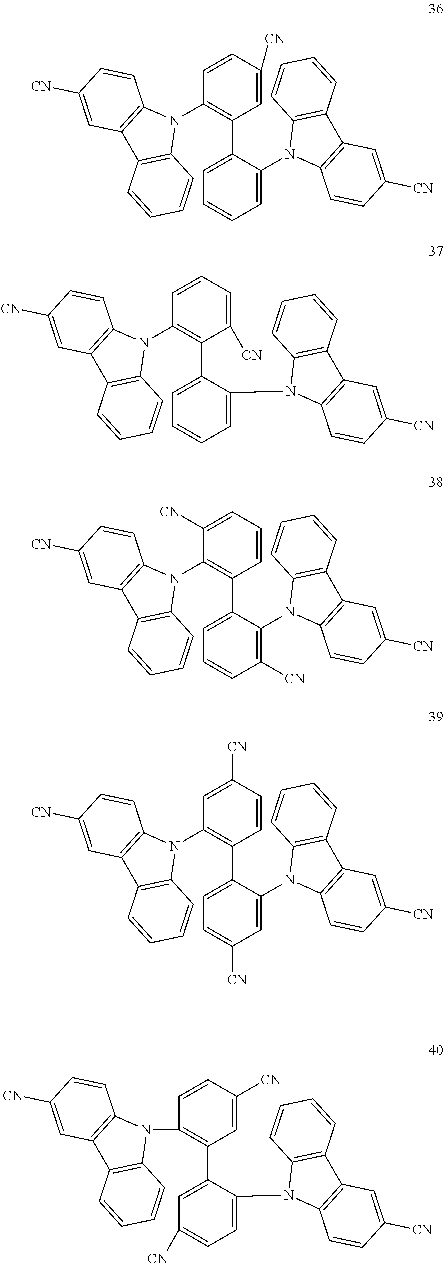

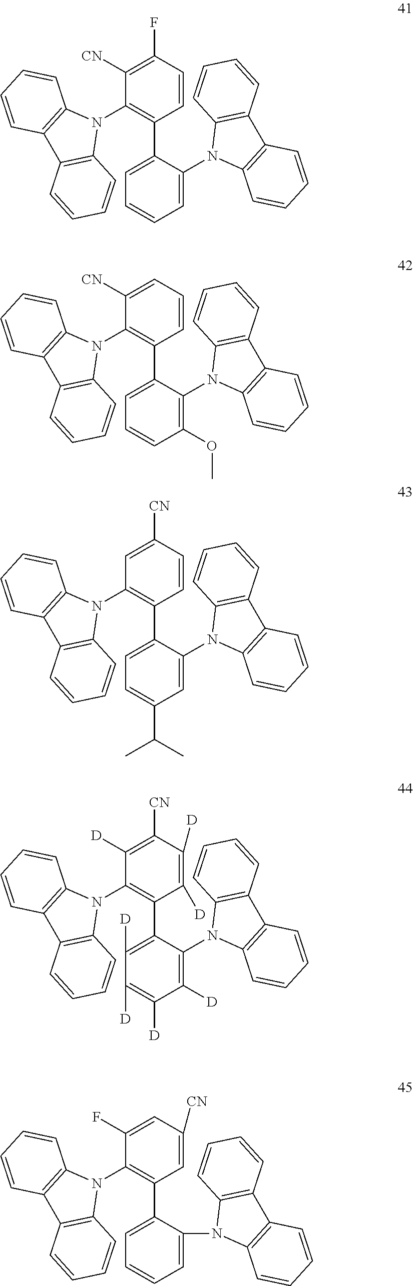

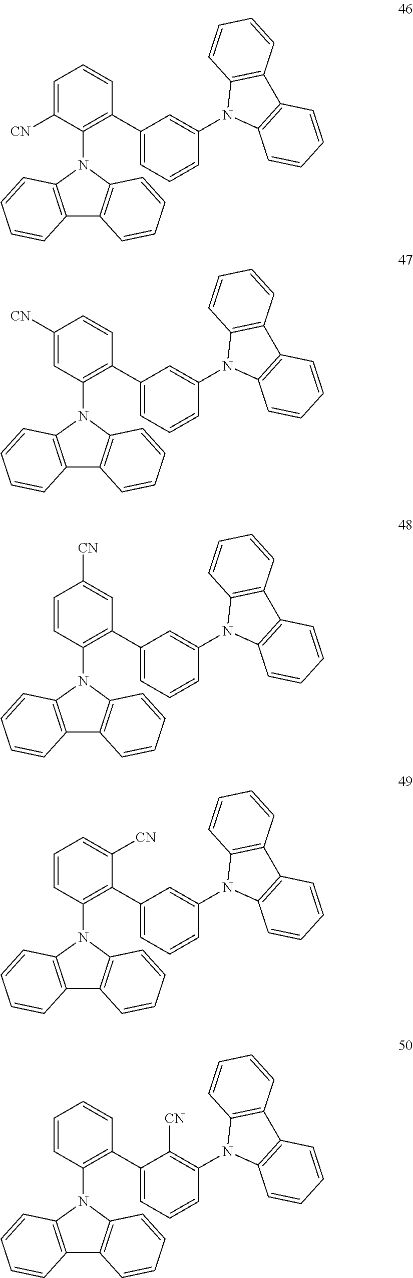

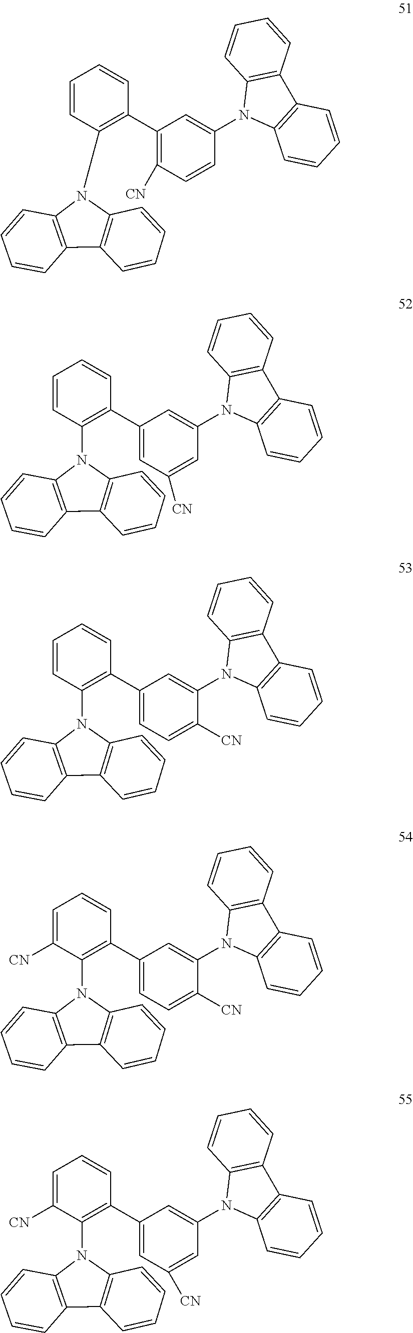

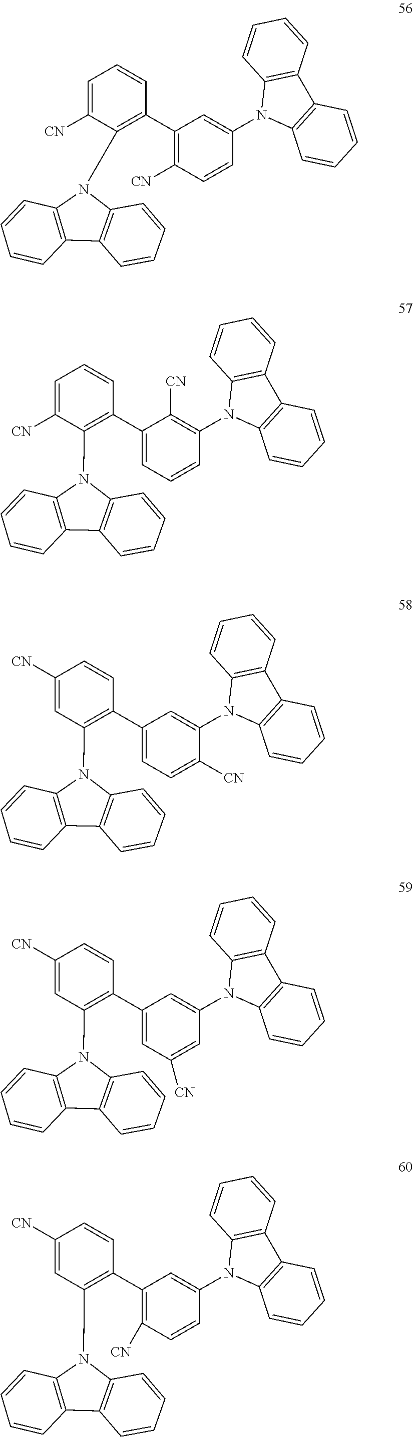

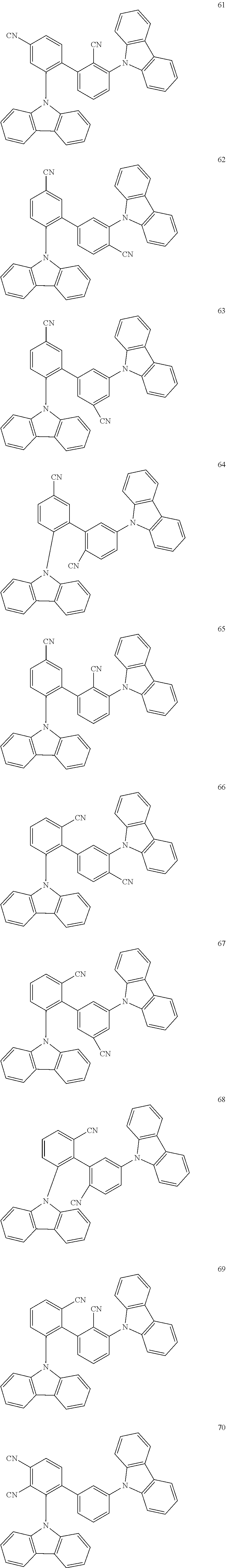

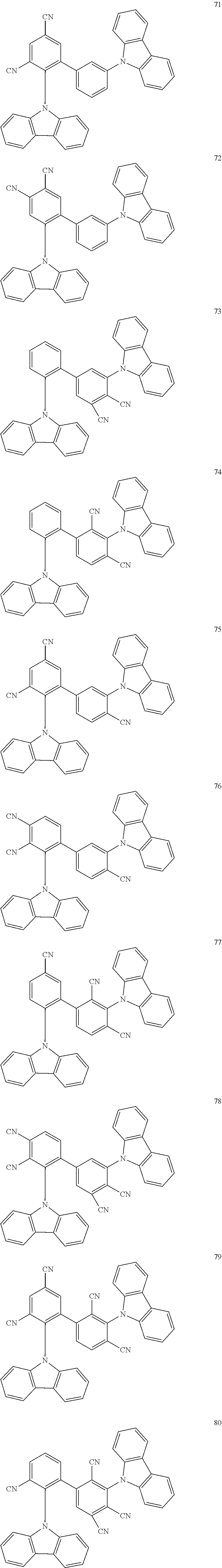

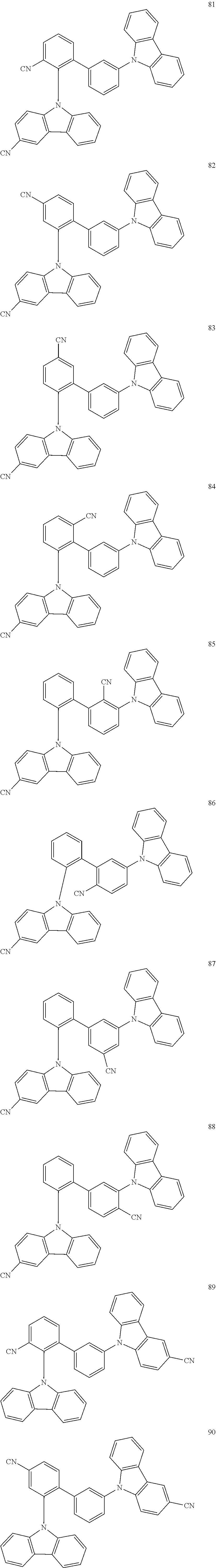

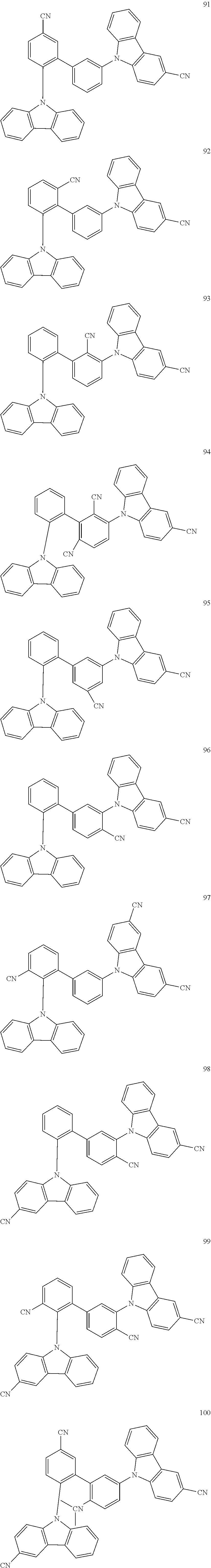

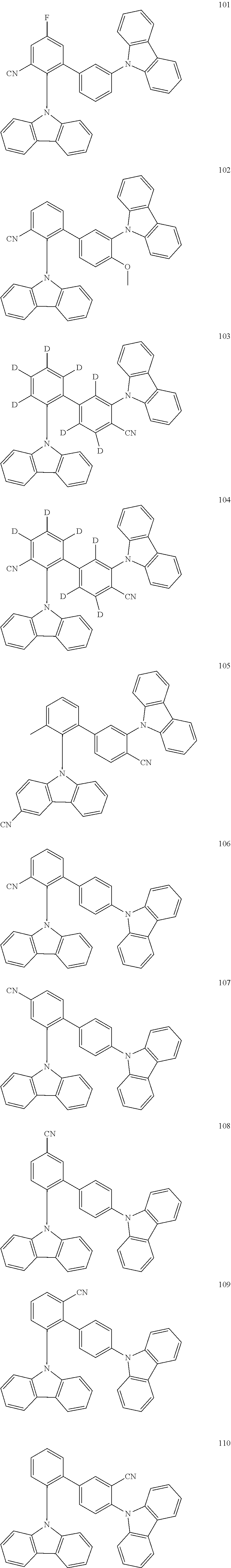

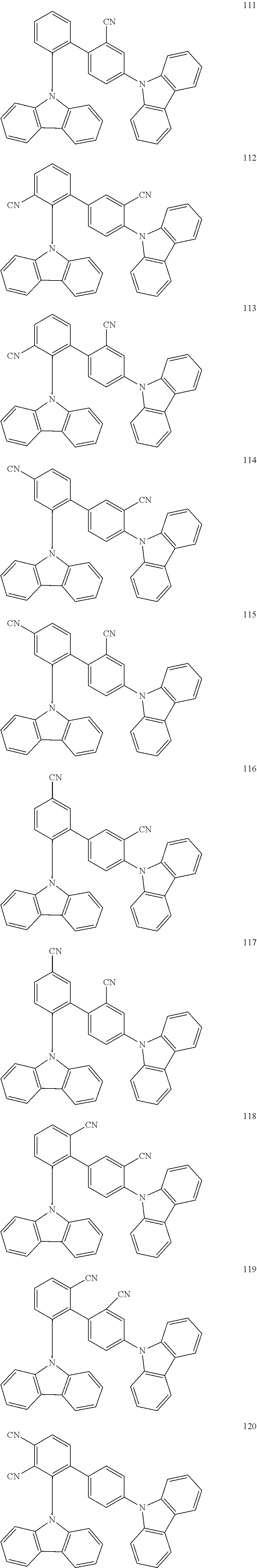

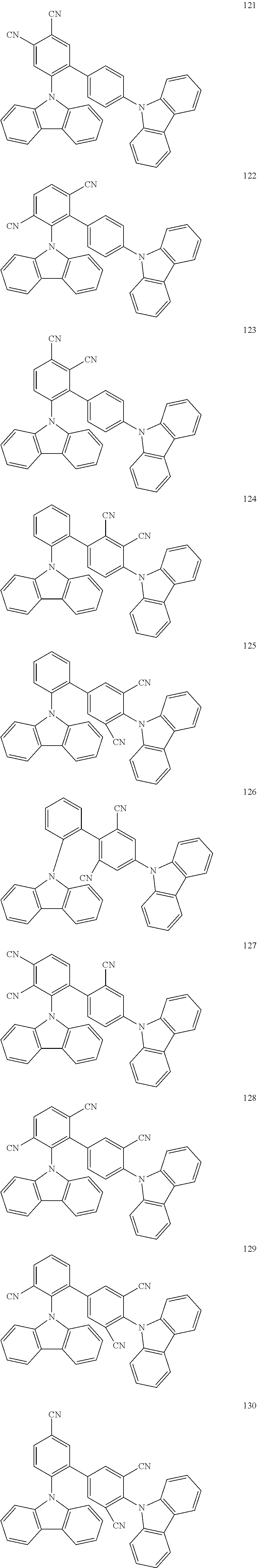

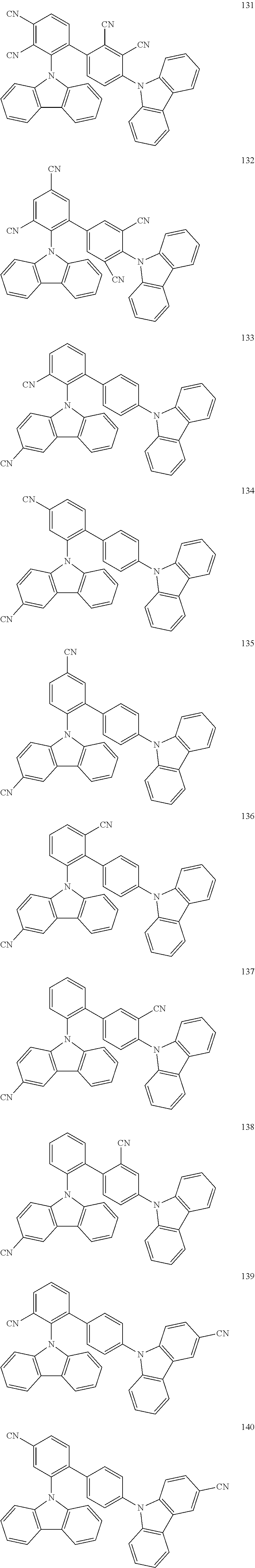

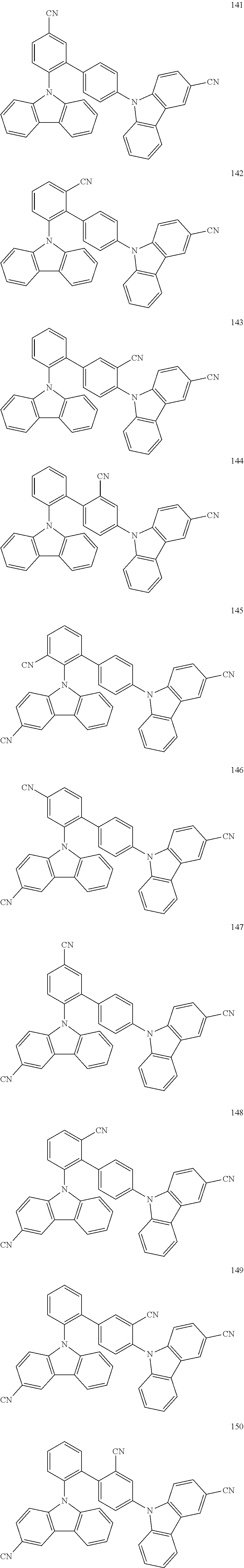

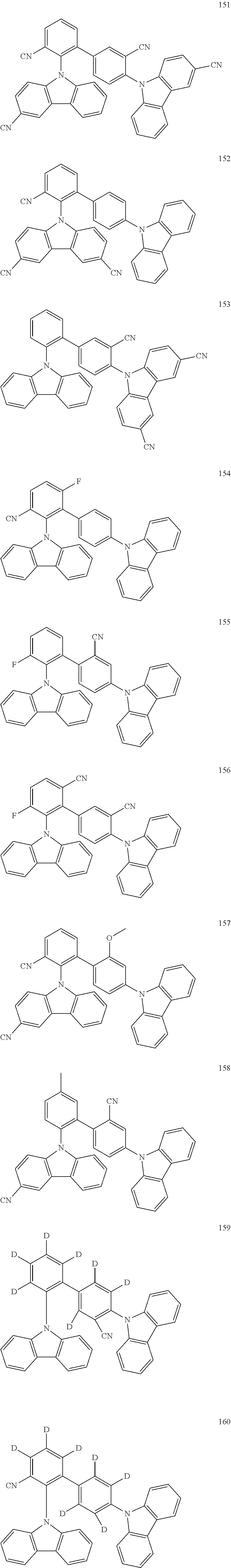

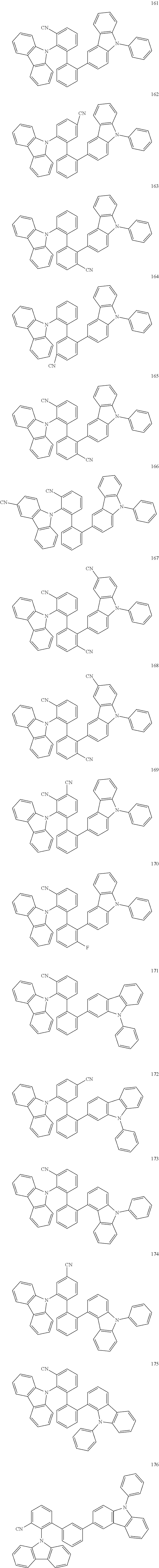

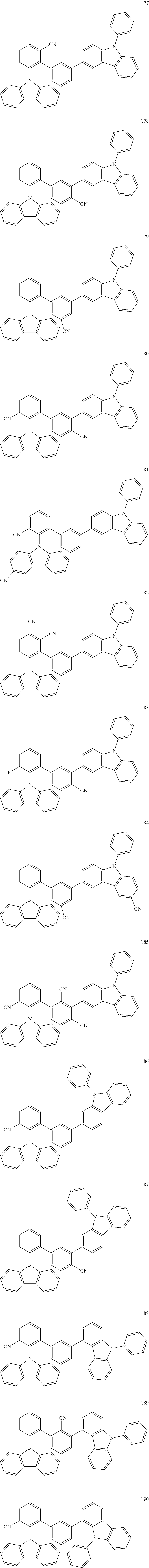

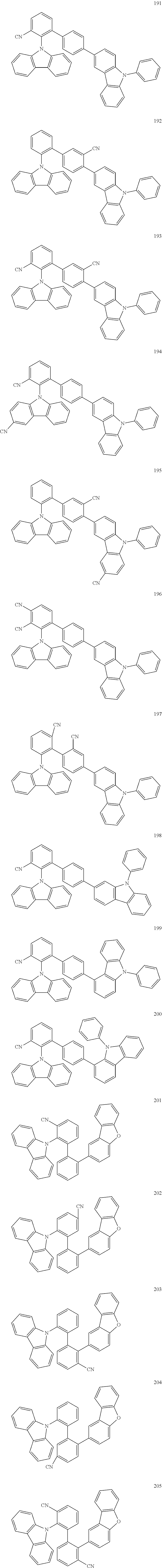

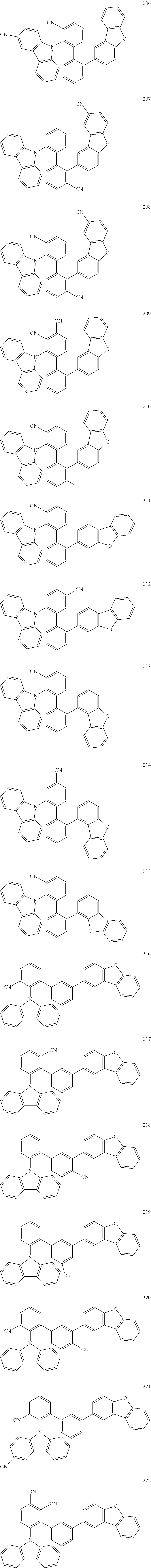

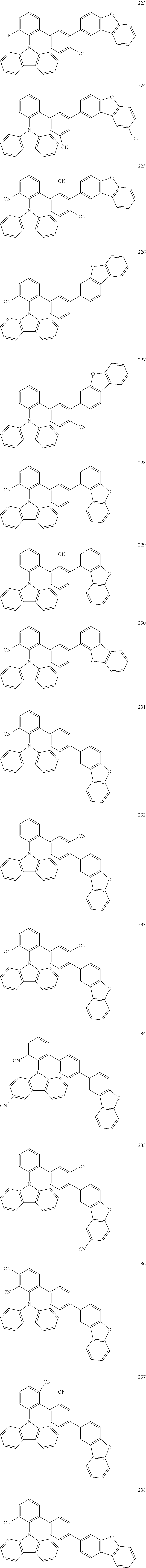

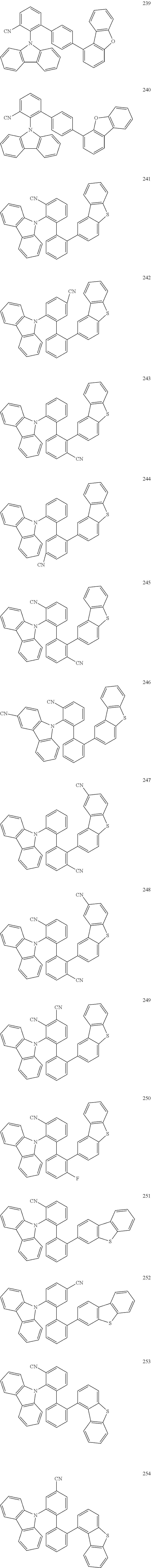

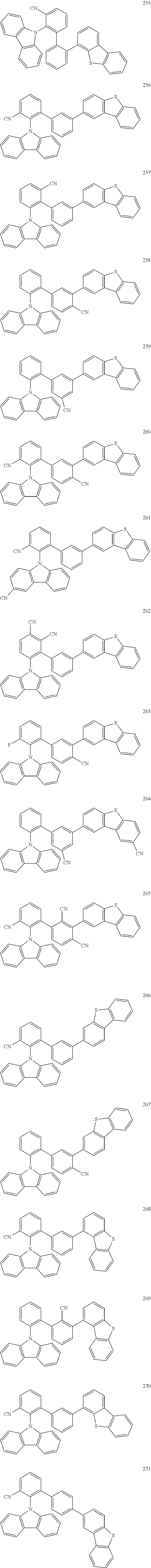

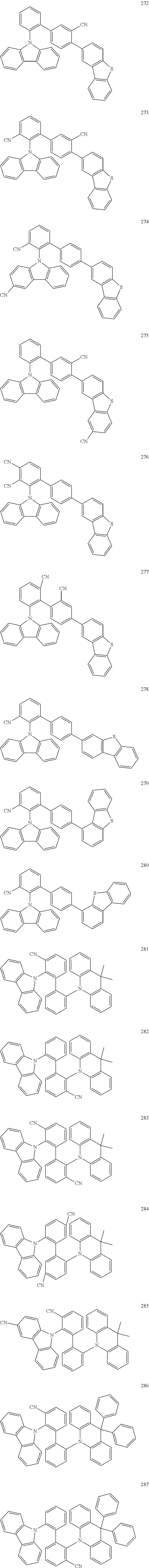

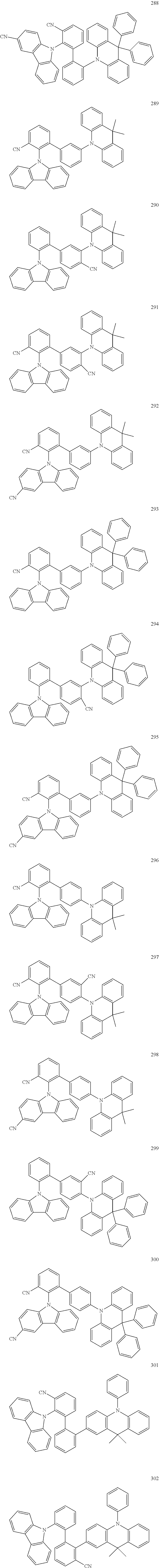

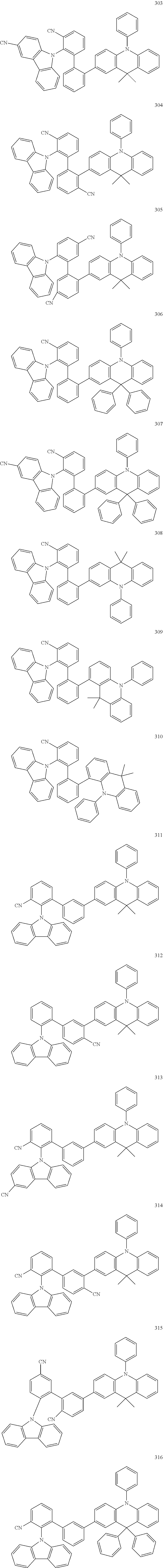

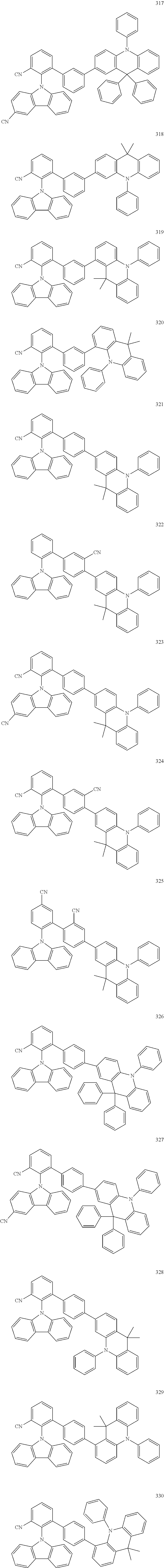

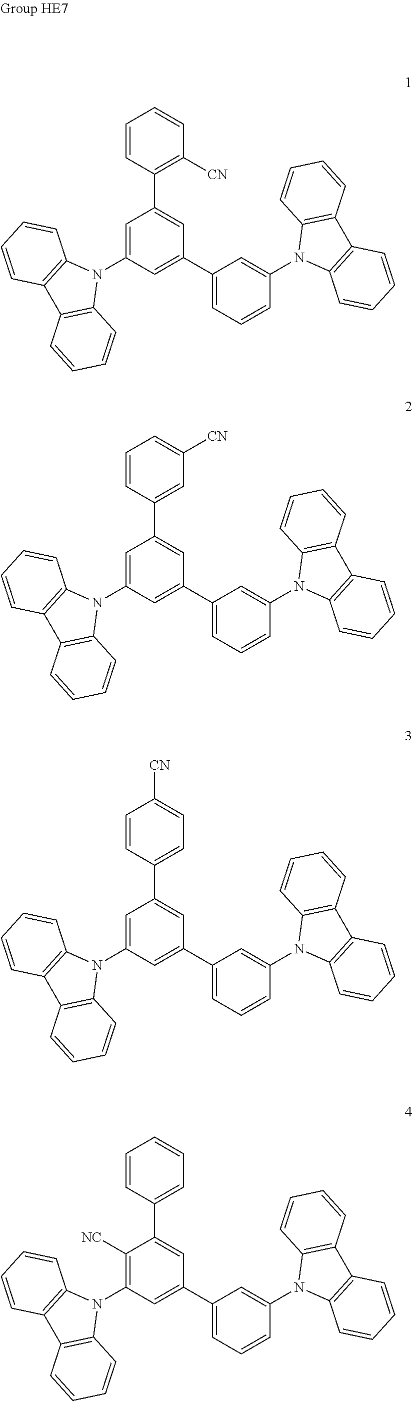 [SMILES: N#Cc1ccccc1-c1cc(-c2cccc(-n3c4ccccc4c4ccccc43)c2)cc(-n2c3ccccc3c3ccccc32)c1 ; N#Cc1cccc(-c2cc(-c3cccc(-n4c5ccccc5c5ccccc54)c3)cc(-n3c4ccccc4c4ccccc43)c2)c1 ; N#Cc1ccc(-c2cc(-c3cccc(-n4c5ccccc5c5ccccc54)c3)cc(-n3c4ccccc4c4ccccc43)c2)cc1 ; N#Cc1c(-c2ccccc2)cc(-c2cccc(-n3c4ccccc4c4ccccc43)c2)cc1-n1c2ccccc2c2ccccc21]

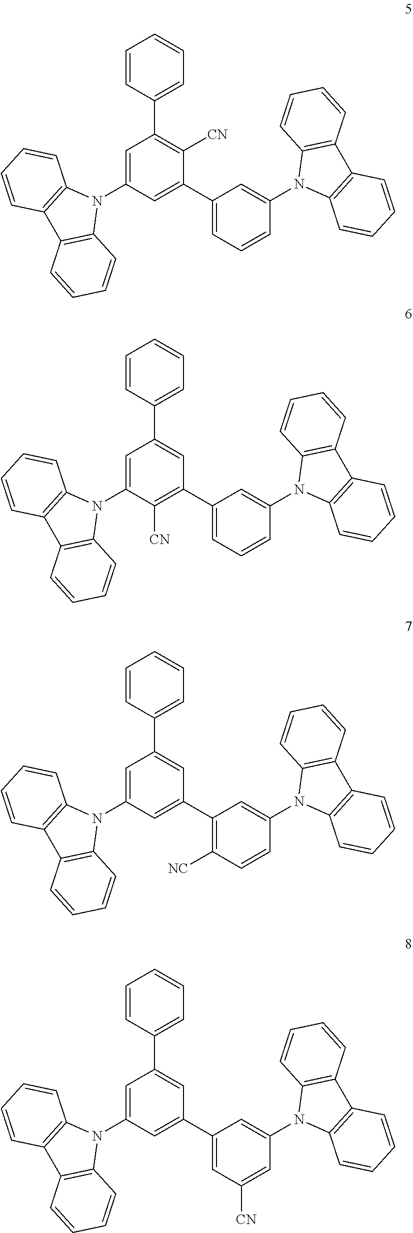 [SMILES: N#Cc1c(-c2ccccc2)cc(-n2c3ccccc3c3ccccc32)cc1-c1cccc(-n2c3ccccc3c3ccccc32)c1 ; N#Cc1c(-c2cccc(-n3c4ccccc4c4ccccc43)c2)cc(-c2ccccc2)cc1-n1c2ccccc2c2ccccc21 ; N#Cc1ccc(-n2c3ccccc3c3ccccc32)cc1-c1cc(-c2ccccc2)cc(-n2c3ccccc3c3ccccc32)c1 ; N#Cc1cc(-c2cc(-c3ccccc3)cc(-n3c4ccccc4c4ccccc43)c2)cc(-n2c3ccccc3c3ccccc32)c1]

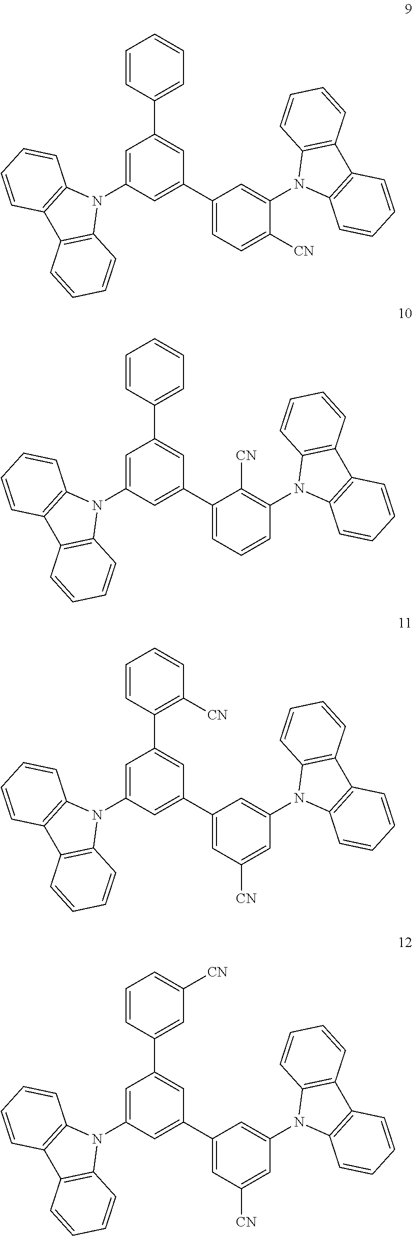 [SMILES: N#Cc1ccc(-c2cc(-c3ccccc3)cc(-n3c4ccccc4c4ccccc43)c2)cc1-n1c2ccccc2c2ccccc21 ; N#Cc1c(-c2cc(-c3ccccc3)cc(-n3c4ccccc4c4ccccc43)c2)cccc1-n1c2ccccc2c2ccccc21 ; N#Cc1cc(-c2cc(-c3ccccc3C#N)cc(-n3c4ccccc4c4ccccc43)c2)cc(-n2c3ccccc3c3ccccc32)c1 ; N#Cc1cccc(-c2cc(-c3cc(C#N)cc(-n4c5ccccc5c5ccccc54)c3)cc(-n3c4ccccc4c4ccccc43)c2)c1]

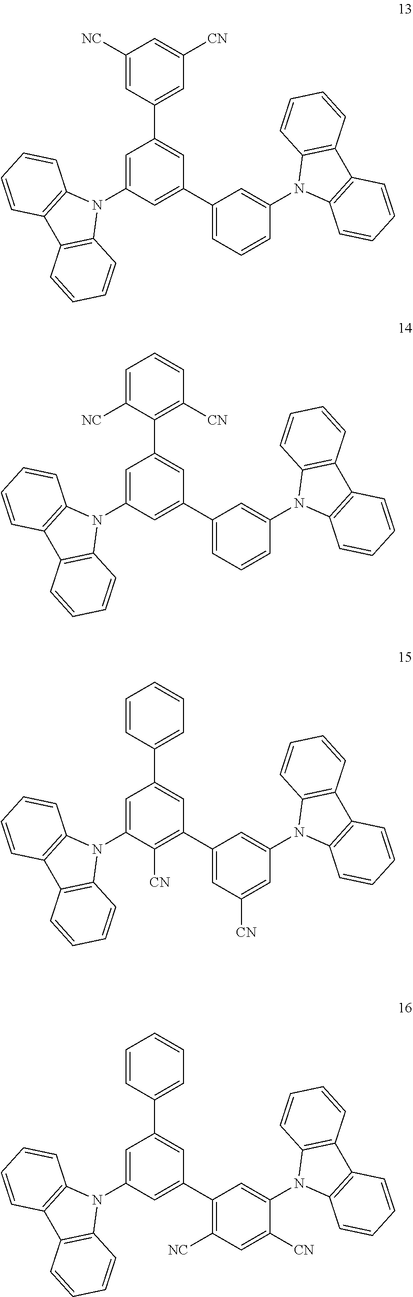 [SMILES: N#Cc1cc(C#N)cc(-c2cc(-c3cccc(-n4c5ccccc5c5ccccc54)c3)cc(-n3c4ccccc4c4ccccc43)c2)c1 ; N#Cc1cccc(C#N)c1-c1cc(-c2cccc(-n3c4ccccc4c4ccccc43)c2)cc(-n2c3ccccc3c3ccccc32)c1 ; N#Cc1cc(-c2cc(-c3ccccc3)cc(-n3c4ccccc4c4ccccc43)c2C#N)cc(-n2c3ccccc3c3ccccc32)c1 ; N#Cc1cc(C#N)c(-n2c3ccccc3c3ccccc32)cc1-c1cc(-c2ccccc2)cc(-n2c3ccccc3c3ccccc32)c1]

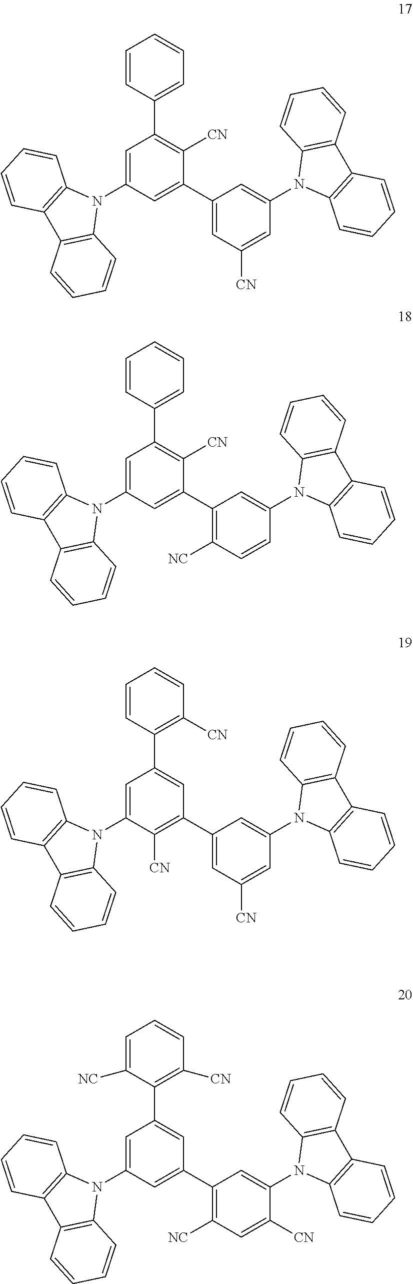 [SMILES: N#Cc1cc(-c2cc(-n3c4ccccc4c4ccccc43)cc(-c3ccccc3)c2C#N)cc(-n2c3ccccc3c3ccccc32)c1 ; N#Cc1ccc(-n2c3ccccc3c3ccccc32)cc1-c1cc(-n2c3ccccc3c3ccccc32)cc(-c2ccccc2)c1C#N ; N#Cc1cc(-c2cc(-c3ccccc3C#N)cc(-n3c4ccccc4c4ccccc43)c2C#N)cc(-n2c3ccccc3c3ccccc32)c1 ; N#Cc1cc(C#N)c(-n2c3ccccc3c3ccccc32)cc1-c1cc(-c2c(C#N)cccc2C#N)cc(-n2c3ccccc3c3ccccc32)c1]

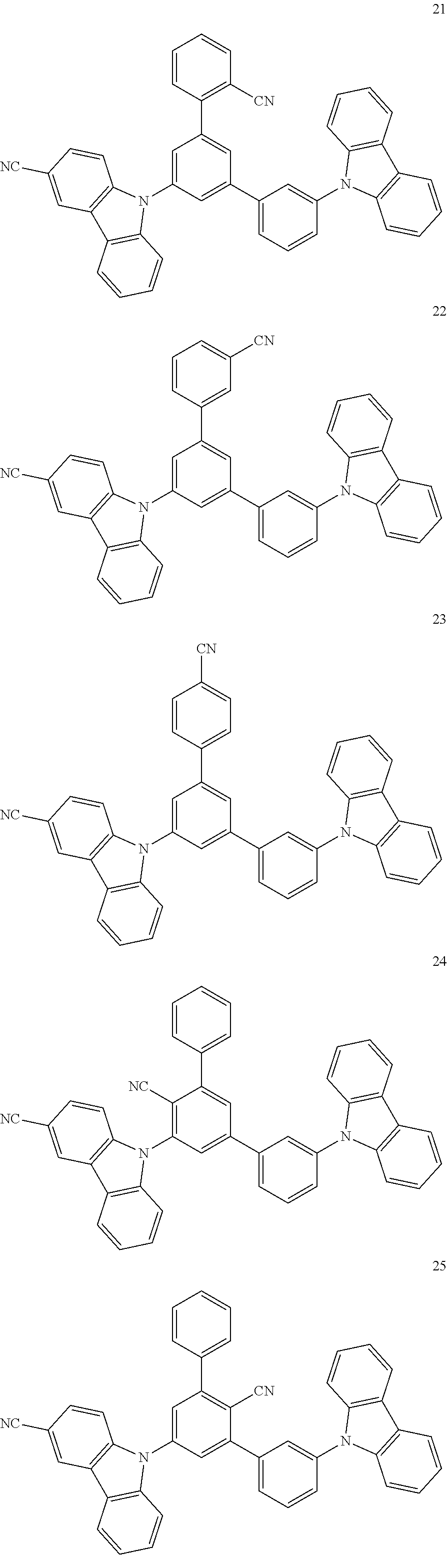 [SMILES: N#Cc1ccc2c(c1)c1ccccc1n2-c1cc(-c2cccc(-n3c4ccccc4c4ccccc43)c2)cc(-c2ccccc2C#N)c1 ; N#Cc1cccc(-c2cc(-c3cccc(-n4c5ccccc5c5ccccc54)c3)cc(-n3c4ccccc4c4cc(C#N)ccc43)c2)c1 ; N#Cc1ccc(-c2cc(-c3cccc(-n4c5ccccc5c5ccccc54)c3)cc(-n3c4ccccc4c4cc(C#N)ccc43)c2)cc1 ; N#Cc1ccc2c(c1)c1ccccc1n2-c1cc(-c2cccc(-n3c4ccccc4c4ccccc43)c2)cc(-c2ccccc2)c1C#N ; N#Cc1ccc2c(c1)c1ccccc1n2-c1cc(-c2ccccc2)c(C#N)c(-c2cccc(-n3c4ccccc4c4ccccc43)c2)c1]

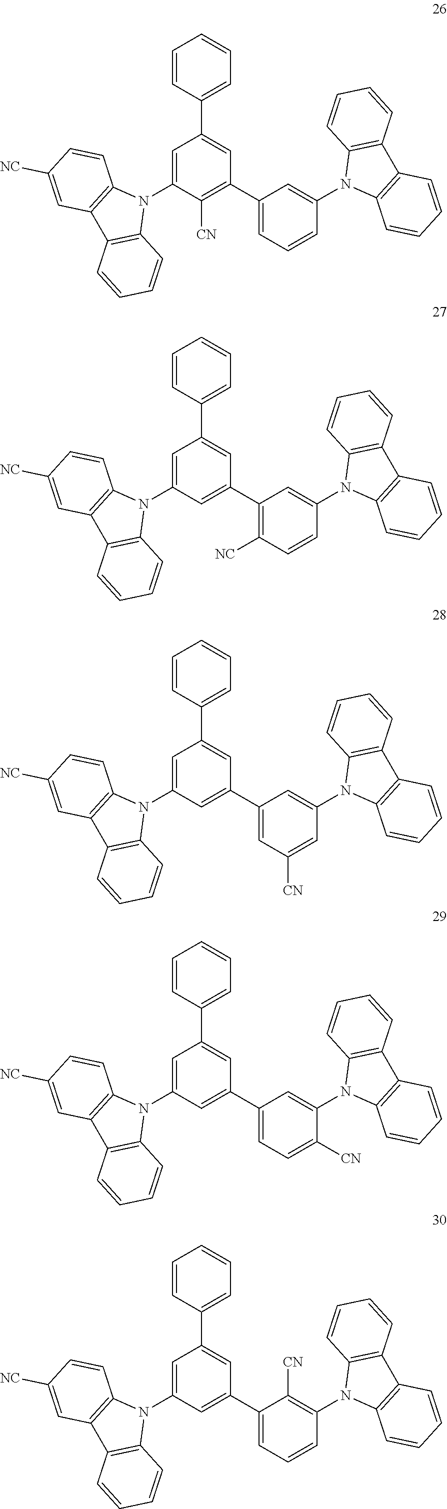 [SMILES: N#Cc1ccc2c(c1)c1ccccc1n2-c1cc(-c2ccccc2)cc(-c2cccc(-n3c4ccccc4c4ccccc43)c2)c1C#N ; N#Cc1ccc2c(c1)c1ccccc1n2-c1cc(-c2ccccc2)cc(-c2cc(-n3c4ccccc4c4ccccc43)ccc2C#N)c1 ; N#Cc1cc(-c2cc(-c3ccccc3)cc(-n3c4ccccc4c4cc(C#N)ccc43)c2)cc(-n2c3ccccc3c3ccccc32)c1 ; N#Cc1ccc2c(c1)c1ccccc1n2-c1cc(-c2ccccc2)cc(-c2ccc(C#N)c(-n3c4ccccc4c4ccccc43)c2)c1 ; N#Cc1ccc2c(c1)c1ccccc1n2-c1cc(-c2ccccc2)cc(-c2cccc(-n3c4ccccc4c4ccccc43)c2C#N)c1]

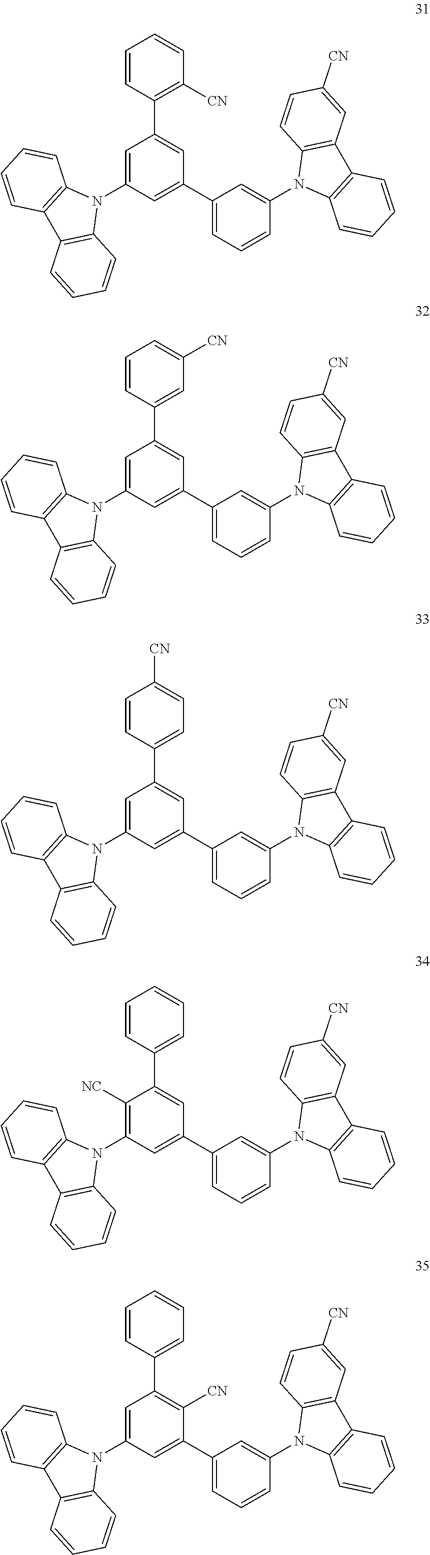 [SMILES: N#Cc1ccc2c(c1)c1ccccc1n2-c1cccc(-c2cc(-c3ccccc3C#N)cc(-n3c4ccccc4c4ccccc43)c2)c1 ; N#Cc1cccc(-c2cc(-c3cccc(-n4c5ccccc5c5cc(C#N)ccc54)c3)cc(-n3c4ccccc4c4ccccc43)c2)c1 ; N#Cc1ccc(-c2cc(-c3cccc(-n4c5ccccc5c5cc(C#N)ccc54)c3)cc(-n3c4ccccc4c4ccccc43)c2)cc1 ; N#Cc1ccc2c(c1)c1ccccc1n2-c1cccc(-c2cc(-c3ccccc3)c(C#N)c(-n3c4ccccc4c4ccccc43)c2)c1 ; N#Cc1ccc2c(c1)c1ccccc1n2-c1cccc(-c2cc(-n3c4ccccc4c4ccccc43)cc(-c3ccccc3)c2C#N)c1]

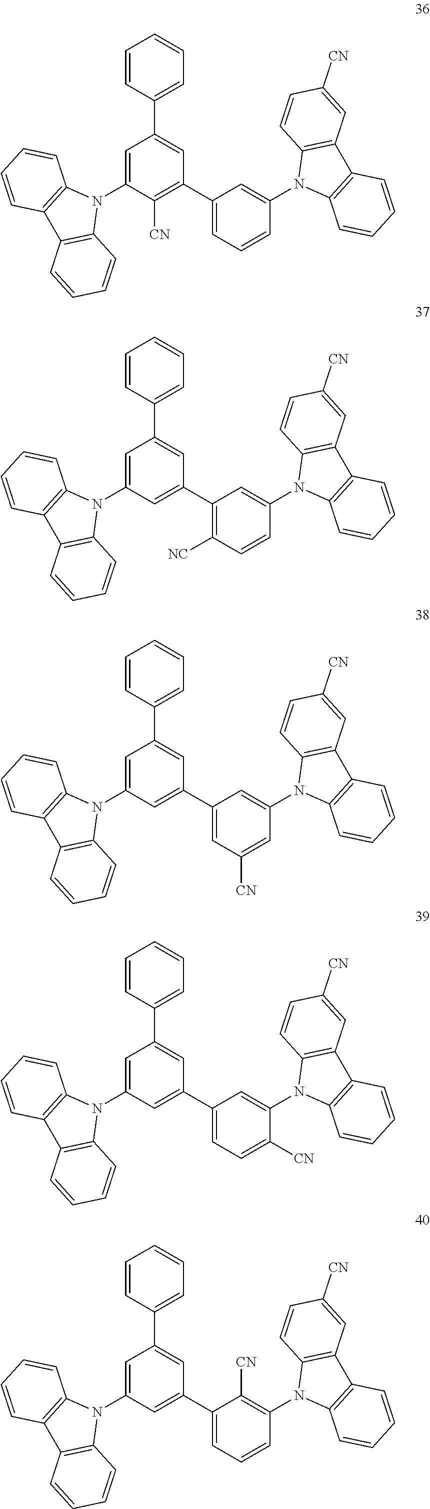 [SMILES: N#Cc1ccc2c(c1)c1ccccc1n2-c1cccc(-c2cc(-c3ccccc3)cc(-n3c4ccccc4c4ccccc43)c2C#N)c1 ; N#Cc1ccc2c(c1)c1ccccc1n2-c1ccc(C#N)c(-c2cc(-c3ccccc3)cc(-n3c4ccccc4c4ccccc43)c2)c1 ; N#Cc1cc(-c2cc(-c3ccccc3)cc(-n3c4ccccc4c4ccccc43)c2)cc(-n2c3ccccc3c3cc(C#N)ccc32)c1 ; N#Cc1ccc2c(c1)c1ccccc1n2-c1cc(-c2cc(-c3ccccc3)cc(-n3c4ccccc4c4ccccc43)c2)ccc1C#N ; N#Cc1ccc2c(c1)c1ccccc1n2-c1cccc(-c2cc(-c3ccccc3)cc(-n3c4ccccc4c4ccccc43)c2)c1C#N]

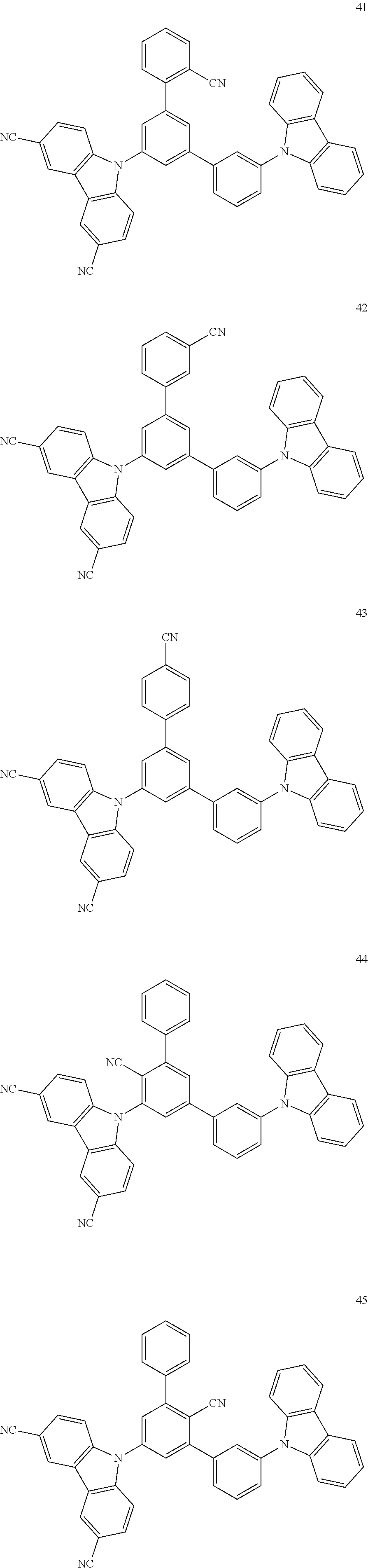 [SMILES: N#Cc1ccc2c(c1)c1cc(C#N)ccc1n2-c1cc(-c2cccc(-n3c4ccccc4c4ccccc43)c2)cc(-c2ccccc2C#N)c1 ; N#Cc1cccc(-c2cc(-c3cccc(-n4c5ccccc5c5ccccc54)c3)cc(-n3c4ccc(C#N)cc4c4cc(C#N)ccc43)c2)c1 ; N#Cc1ccc(-c2cc(-c3cccc(-n4c5ccccc5c5ccccc54)c3)cc(-n3c4ccc(C#N)cc4c4cc(C#N)ccc43)c2)cc1 ; N#Cc1ccc2c(c1)c1cc(C#N)ccc1n2-c1cc(-c2cccc(-n3c4ccccc4c4ccccc43)c2)cc(-c2ccccc2)c1C#N ; N#Cc1ccc2c(c1)c1cc(C#N)ccc1n2-c1cc(-c2ccccc2)c(C#N)c(-c2cccc(-n3c4ccccc4c4ccccc43)c2)c1]

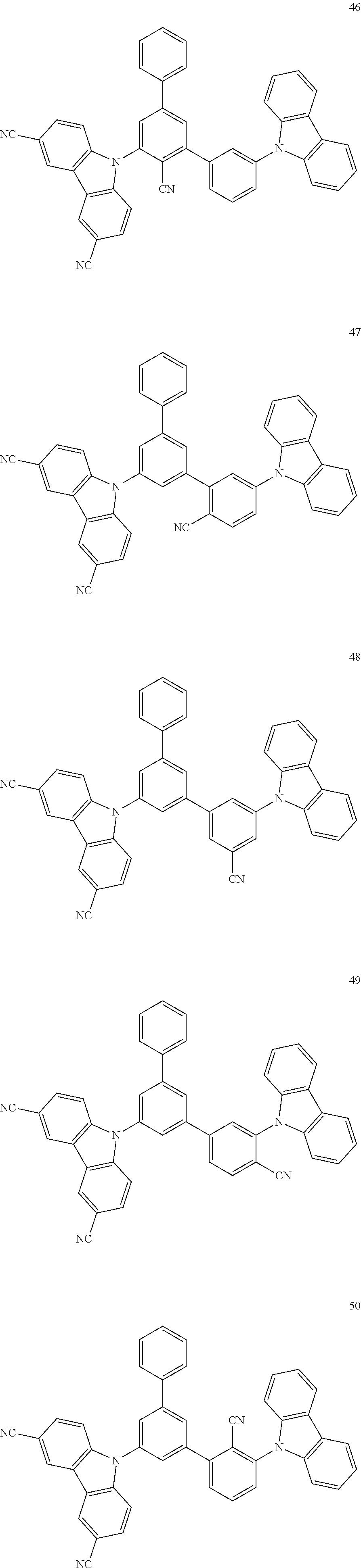 [SMILES: N#Cc1ccc2c(c1)c1cc(C#N)ccc1n2-c1cc(-c2ccccc2)cc(-c2cccc(-n3c4ccccc4c4ccccc43)c2)c1C#N ; N#Cc1ccc2c(c1)c1cc(C#N)ccc1n2-c1cc(-c2ccccc2)cc(-c2cc(-n3c4ccccc4c4ccccc43)ccc2C#N)c1 ; N#Cc1cc(-c2cc(-c3ccccc3)cc(-n3c4ccc(C#N)cc4c4cc(C#N)ccc43)c2)cc(-n2c3ccccc3c3ccccc32)c1 ; N#Cc1ccc2c(c1)c1cc(C#N)ccc1n2-c1cc(-c2ccccc2)cc(-c2ccc(C#N)c(-n3c4ccccc4c4ccccc43)c2)c1 ; N#Cc1ccc2c(c1)c1cc(C#N)ccc1n2-c1cc(-c2ccccc2)cc(-c2cccc(-n3c4ccccc4c4ccccc43)c2C#N)c1]

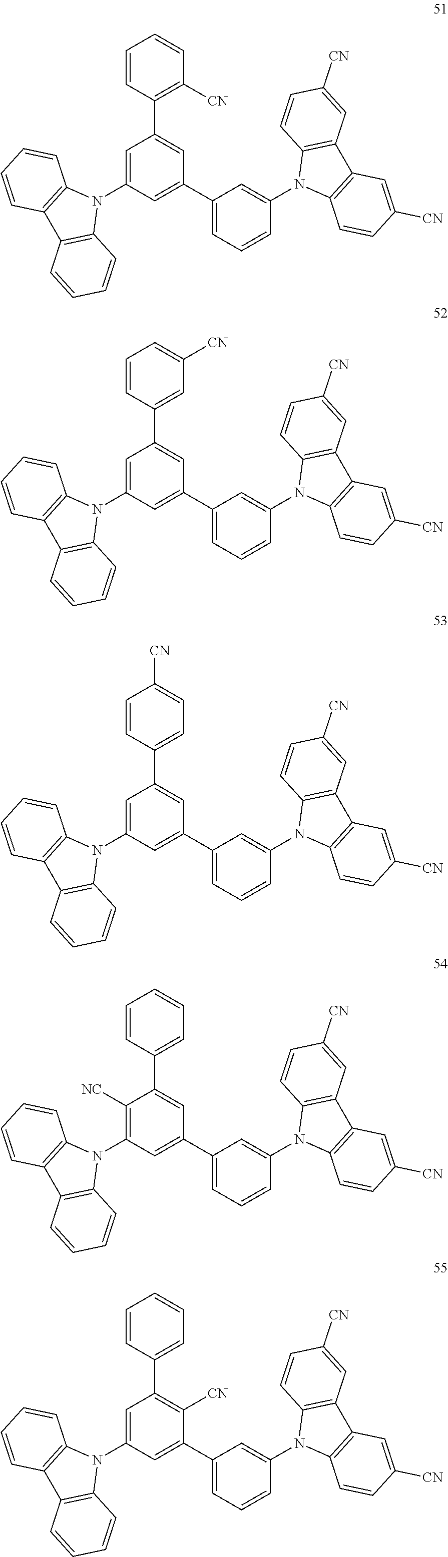 [SMILES: N#Cc1ccc2c(c1)c1cc(C#N)ccc1n2-c1cccc(-c2cc(-c3ccccc3C#N)cc(-n3c4ccccc4c4ccccc43)c2)c1 ; N#Cc1cccc(-c2cc(-c3cccc(-n4c5ccc(C#N)cc5c5cc(C#N)ccc54)c3)cc(-n3c4ccccc4c4ccccc43)c2)c1 ; N#Cc1ccc(-c2cc(-c3cccc(-n4c5ccc(C#N)cc5c5cc(C#N)ccc54)c3)cc(-n3c4ccccc4c4ccccc43)c2)cc1 ; N#Cc1ccc2c(c1)c1cc(C#N)ccc1n2-c1cccc(-c2cc(-c3ccccc3)c(C#N)c(-n3c4ccccc4c4ccccc43)c2)c1 ; N#Cc1ccc2c(c1)c1cc(C#N)ccc1n2-c1cccc(-c2cc(-n3c4ccccc4c4ccccc43)cc(-c3ccccc3)c2C#N)c1]

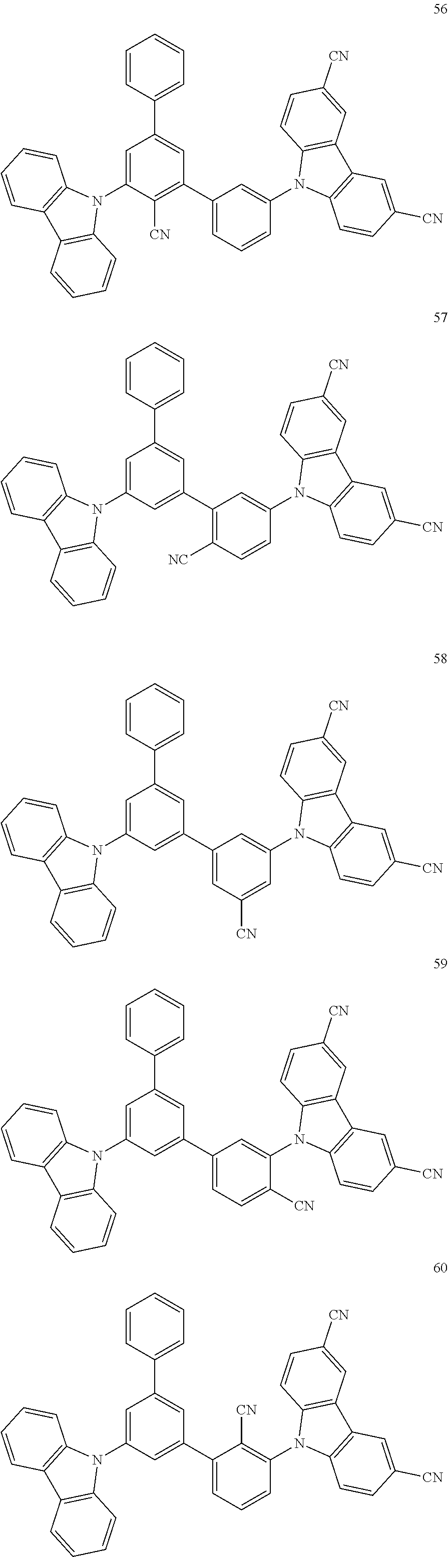 [SMILES: N#Cc1ccc2c(c1)c1cc(C#N)ccc1n2-c1cccc(-c2cc(-c3ccccc3)cc(-n3c4ccccc4c4ccccc43)c2C#N)c1 ; N#Cc1ccc2c(c1)c1cc(C#N)ccc1n2-c1ccc(C#N)c(-c2cc(-c3ccccc3)cc(-n3c4ccccc4c4ccccc43)c2)c1 ; N#Cc1cc(-c2cc(-c3ccccc3)cc(-n3c4ccccc4c4ccccc43)c2)cc(-n2c3ccc(C#N)cc3c3cc(C#N)ccc32)c1 ; N#Cc1ccc2c(c1)c1cc(C#N)ccc1n2-c1cc(-c2cc(-c3ccccc3)cc(-n3c4ccccc4c4ccccc43)c2)ccc1C#N ; N#Cc1ccc2c(c1)c1cc(C#N)ccc1n2-c1cccc(-c2cc(-c3ccccc3)cc(-n3c4ccccc4c4ccccc43)c2)c1C#N]

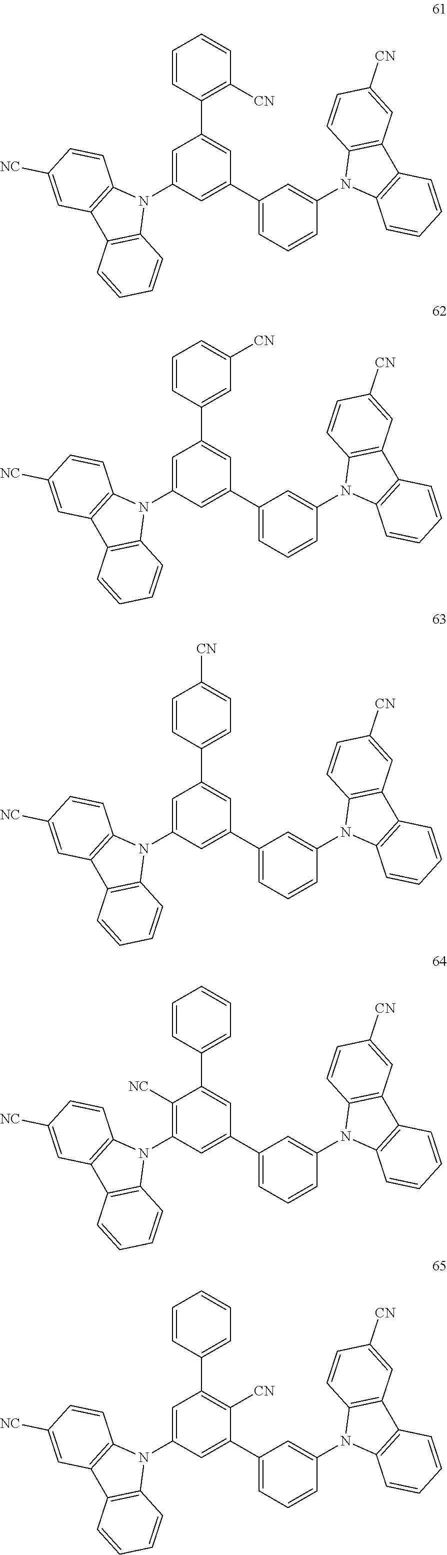 [SMILES: N#Cc1ccc2c(c1)c1ccccc1n2-c1cccc(-c2cc(-c3ccccc3C#N)cc(-n3c4ccccc4c4cc(C#N)ccc43)c2)c1 ; N#Cc1cccc(-c2cc(-c3cccc(-n4c5ccccc5c5cc(C#N)ccc54)c3)cc(-n3c4ccccc4c4cc(C#N)ccc43)c2)c1 ; N#Cc1ccc(-c2cc(-c3cccc(-n4c5ccccc5c5cc(C#N)ccc54)c3)cc(-n3c4ccccc4c4cc(C#N)ccc43)c2)cc1 ; N#Cc1ccc2c(c1)c1ccccc1n2-c1cccc(-c2cc(-c3ccccc3)c(C#N)c(-n3c4ccccc4c4cc(C#N)ccc43)c2)c1 ; N#Cc1ccc2c(c1)c1ccccc1n2-c1cccc(-c2cc(-n3c4ccccc4c4cc(C#N)ccc43)cc(-c3ccccc3)c2C#N)c1]

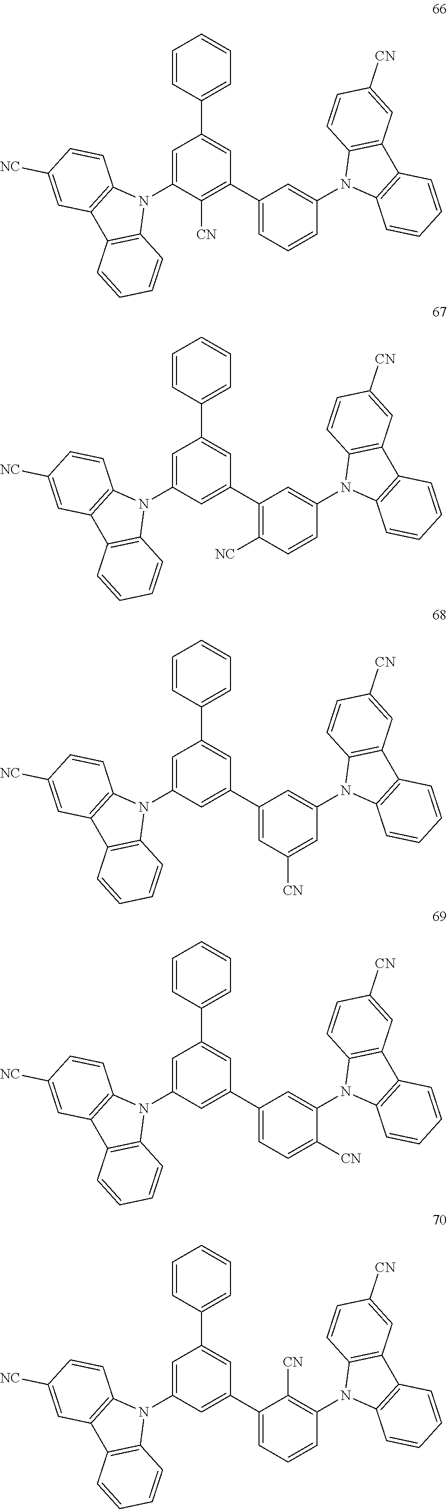 [SMILES: N#Cc1ccc2c(c1)c1ccccc1n2-c1cccc(-c2cc(-c3ccccc3)cc(-n3c4ccccc4c4cc(C#N)ccc43)c2C#N)c1 ; N#Cc1ccc2c(c1)c1ccccc1n2-c1cc(-c2ccccc2)cc(-c2cc(-n3c4ccccc4c4cc(C#N)ccc43)ccc2C#N)c1 ; N#Cc1cc(-c2cc(-c3ccccc3)cc(-n3c4ccccc4c4cc(C#N)ccc43)c2)cc(-n2c3ccccc3c3cc(C#N)ccc32)c1 ; N#Cc1ccc2c(c1)c1ccccc1n2-c1cc(-c2ccccc2)cc(-c2ccc(C#N)c(-n3c4ccccc4c4cc(C#N)ccc43)c2)c1 ; N#Cc1ccc2c(c1)c1ccccc1n2-c1cc(-c2ccccc2)cc(-c2cccc(-n3c4ccccc4c4cc(C#N)ccc43)c2C#N)c1]

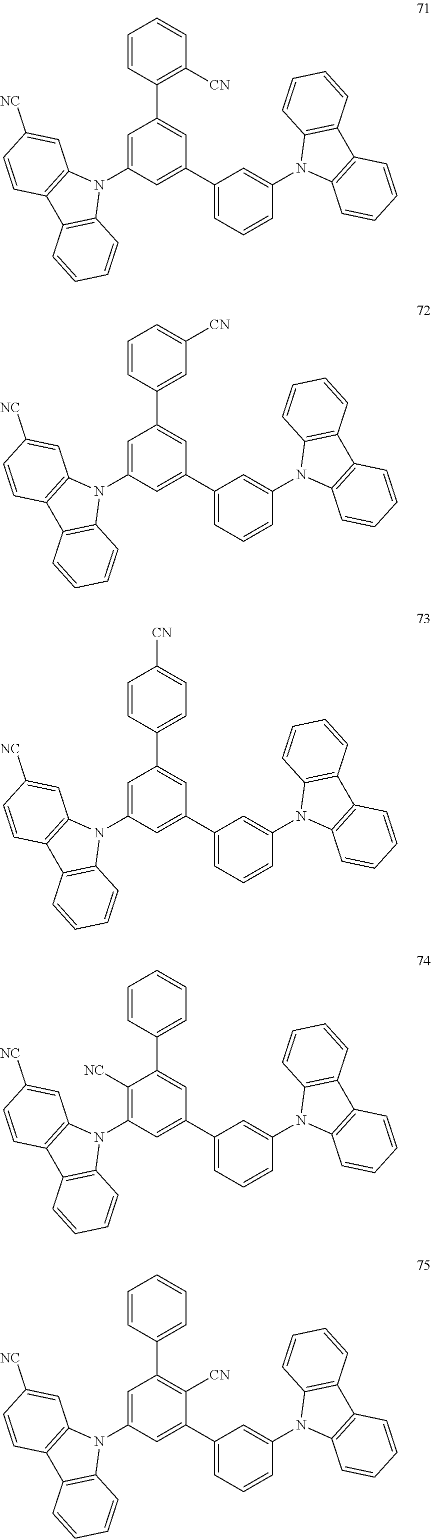 [SMILES: N#Cc1ccc2c3ccccc3n(-c3cc(-c4cccc(-n5c6ccccc6c6ccccc65)c4)cc(-c4ccccc4C#N)c3)c2c1 ; N#Cc1cccc(-c2cc(-c3cccc(-n4c5ccccc5c5ccccc54)c3)cc(-n3c4ccccc4c4ccc(C#N)cc43)c2)c1 ; N#Cc1ccc(-c2cc(-c3cccc(-n4c5ccccc5c5ccccc54)c3)cc(-n3c4ccccc4c4ccc(C#N)cc43)c2)cc1 ; N#Cc1ccc2c3ccccc3n(-c3cc(-c4cccc(-n5c6ccccc6c6ccccc65)c4)cc(-c4ccccc4)c3C#N)c2c1 ; N#Cc1ccc2c3ccccc3n(-c3cc(-c4ccccc4)c(C#N)c(-c4cccc(-n5c6ccccc6c6ccccc65)c4)c3)c2c1]

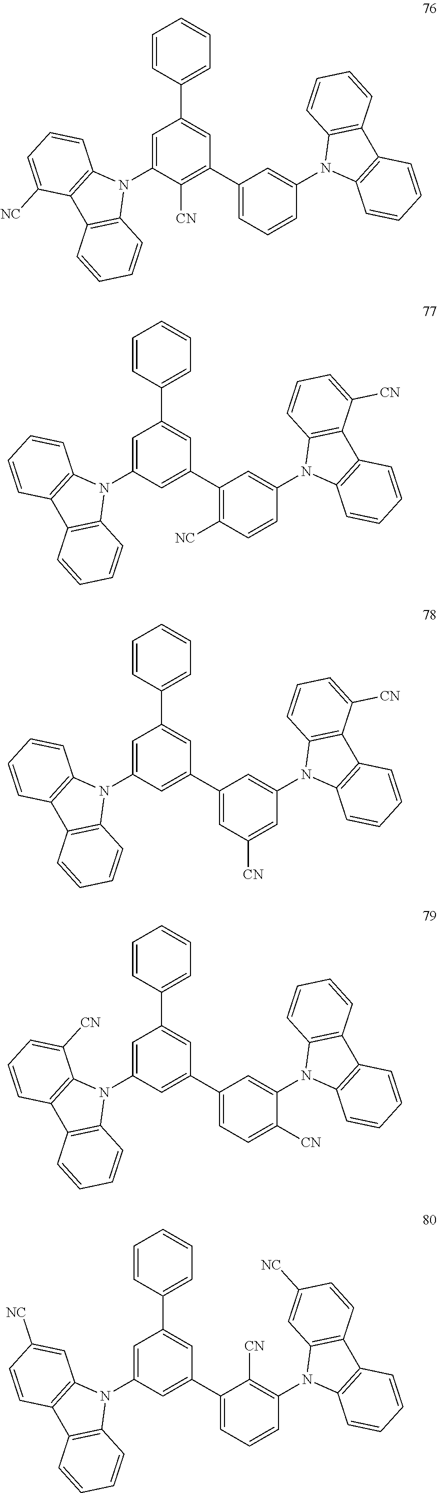 [SMILES: N#Cc1c(-c2cccc(-n3c4ccccc4c4ccccc43)c2)cc(-c2ccccc2)cc1-n1c2ccccc2c2c(C#N)cccc21 ; N#Cc1ccc(-n2c3ccccc3c3c(C#N)cccc32)cc1-c1cc(-c2ccccc2)cc(-n2c3ccccc3c3ccccc32)c1 ; N#Cc1cc(-c2cc(-c3ccccc3)cc(-n3c4ccccc4c4ccccc43)c2)cc(-n2c3ccccc3c3c(C#N)cccc32)c1 ; N#Cc1ccc(-c2cc(-c3ccccc3)cc(-n3c4ccccc4c4cccc(C#N)c43)c2)cc1-n1c2ccccc2c2ccccc21 ; N#Cc1ccc2c3ccccc3n(-c3cc(-c4ccccc4)cc(-c4cccc(-n5c6ccccc6c6ccc(C#N)cc65)c4C#N)c3)c2c1]

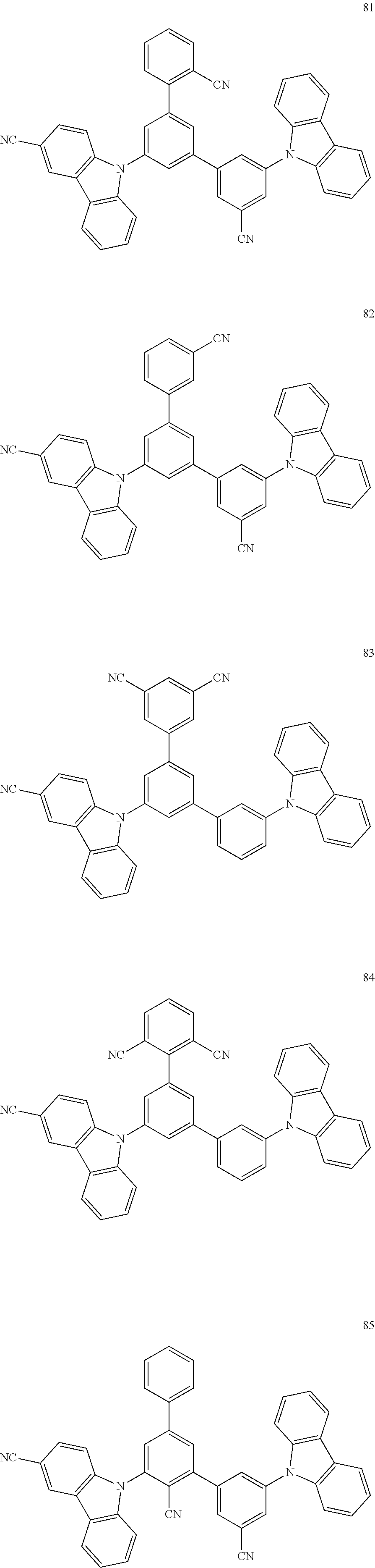 [SMILES: N#Cc1cc(-c2cc(-c3ccccc3C#N)cc(-n3c4ccccc4c4cc(C#N)ccc43)c2)cc(-n2c3ccccc3c3ccccc32)c1 ; N#Cc1cccc(-c2cc(-c3cc(C#N)cc(-n4c5ccccc5c5ccccc54)c3)cc(-n3c4ccccc4c4cc(C#N)ccc43)c2)c1 ; N#Cc1cc(C#N)cc(-c2cc(-c3cccc(-n4c5ccccc5c5ccccc54)c3)cc(-n3c4ccccc4c4cc(C#N)ccc43)c2)c1 ; N#Cc1ccc2c(c1)c1ccccc1n2-c1cc(-c2cccc(-n3c4ccccc4c4ccccc43)c2)cc(-c2c(C#N)cccc2C#N)c1 ; N#Cc1cc(-c2cc(-c3ccccc3)cc(-n3c4ccccc4c4cc(C#N)ccc43)c2C#N)cc(-n2c3ccccc3c3ccccc32)c1]

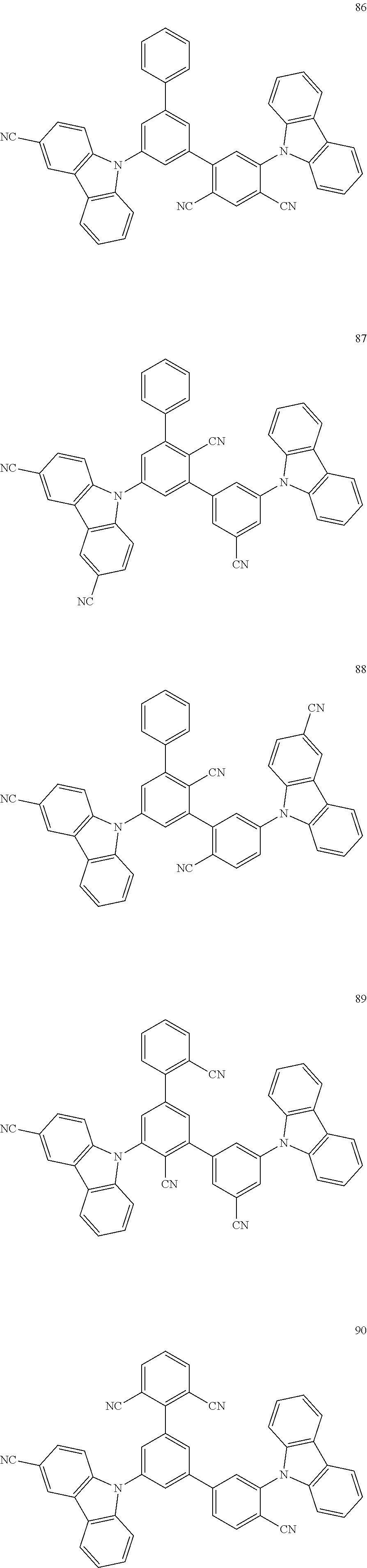 [SMILES: N#Cc1ccc2c(c1)c1ccccc1n2-c1cc(-c2ccccc2)cc(-c2cc(-n3c4ccccc4c4ccccc43)c(C#N)cc2C#N)c1 ; N#Cc1cc(-c2cc(-n3c4ccc(C#N)cc4c4cc(C#N)ccc43)cc(-c3ccccc3)c2C#N)cc(-n2c3ccccc3c3ccccc32)c1 ; N#Cc1ccc2c(c1)c1ccccc1n2-c1ccc(C#N)c(-c2cc(-n3c4ccccc4c4cc(C#N)ccc43)cc(-c3ccccc3)c2C#N)c1 ; N#Cc1cc(-c2cc(-c3ccccc3C#N)cc(-n3c4ccccc4c4cc(C#N)ccc43)c2C#N)cc(-n2c3ccccc3c3ccccc32)c1 ; N#Cc1ccc2c(c1)c1ccccc1n2-c1cc(-c2ccc(C#N)c(-n3c4ccccc4c4ccccc43)c2)cc(-c2c(C#N)cccc2C#N)c1]

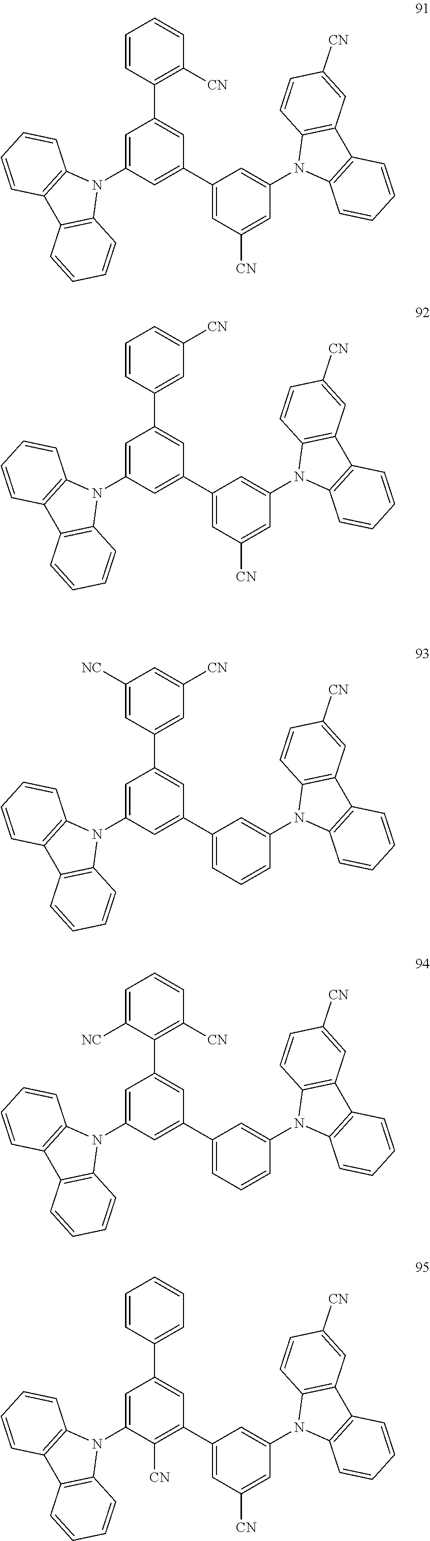 [SMILES: N#Cc1cc(-c2cc(-c3ccccc3C#N)cc(-n3c4ccccc4c4ccccc43)c2)cc(-n2c3ccccc3c3cc(C#N)ccc32)c1 ; N#Cc1cccc(-c2cc(-c3cc(C#N)cc(-n4c5ccccc5c5cc(C#N)ccc54)c3)cc(-n3c4ccccc4c4ccccc43)c2)c1 ; N#Cc1cc(C#N)cc(-c2cc(-c3cccc(-n4c5ccccc5c5cc(C#N)ccc54)c3)cc(-n3c4ccccc4c4ccccc43)c2)c1 ; N#Cc1ccc2c(c1)c1ccccc1n2-c1cccc(-c2cc(-c3c(C#N)cccc3C#N)cc(-n3c4ccccc4c4ccccc43)c2)c1 ; N#Cc1cc(-c2cc(-c3ccccc3)cc(-n3c4ccccc4c4ccccc43)c2C#N)cc(-n2c3ccccc3c3cc(C#N)ccc32)c1]

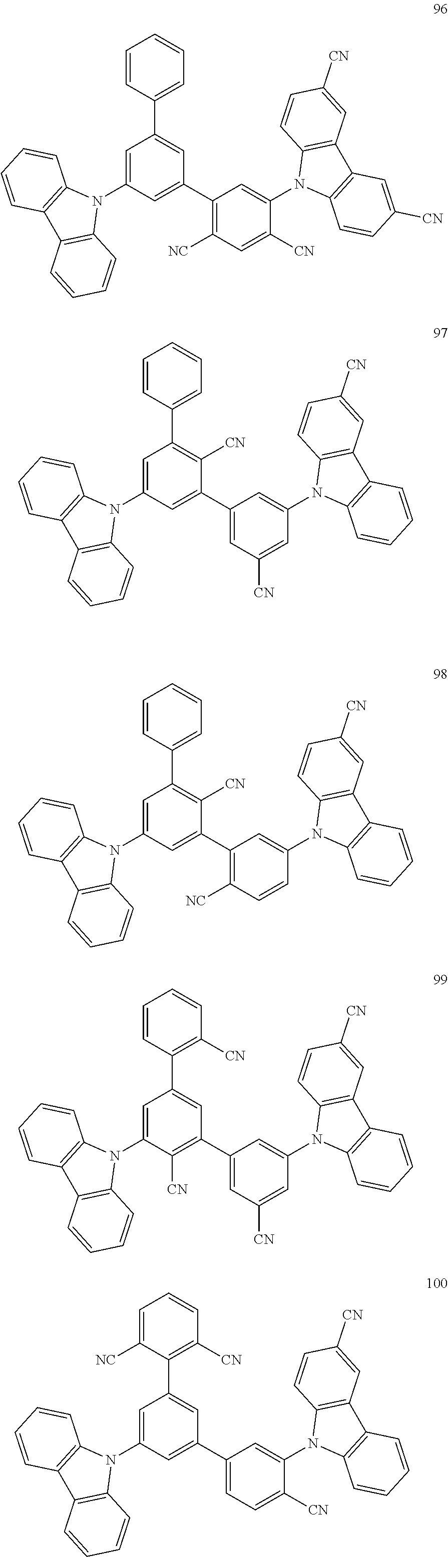 [SMILES: N#Cc1ccc2c(c1)c1cc(C#N)ccc1n2-c1cc(-c2cc(-c3ccccc3)cc(-n3c4ccccc4c4ccccc43)c2)c(C#N)cc1C#N ; N#Cc1cc(-c2cc(-n3c4ccccc4c4ccccc43)cc(-c3ccccc3)c2C#N)cc(-n2c3ccccc3c3cc(C#N)ccc32)c1 ; N#Cc1ccc2c(c1)c1ccccc1n2-c1ccc(C#N)c(-c2cc(-n3c4ccccc4c4ccccc43)cc(-c3ccccc3)c2C#N)c1 ; N#Cc1cc(-c2cc(-c3ccccc3C#N)cc(-n3c4ccccc4c4ccccc43)c2C#N)cc(-n2c3ccccc3c3cc(C#N)ccc32)c1 ; N#Cc1ccc2c(c1)c1ccccc1n2-c1cc(-c2cc(-c3c(C#N)cccc3C#N)cc(-n3c4ccccc4c4ccccc43)c2)ccc1C#N]

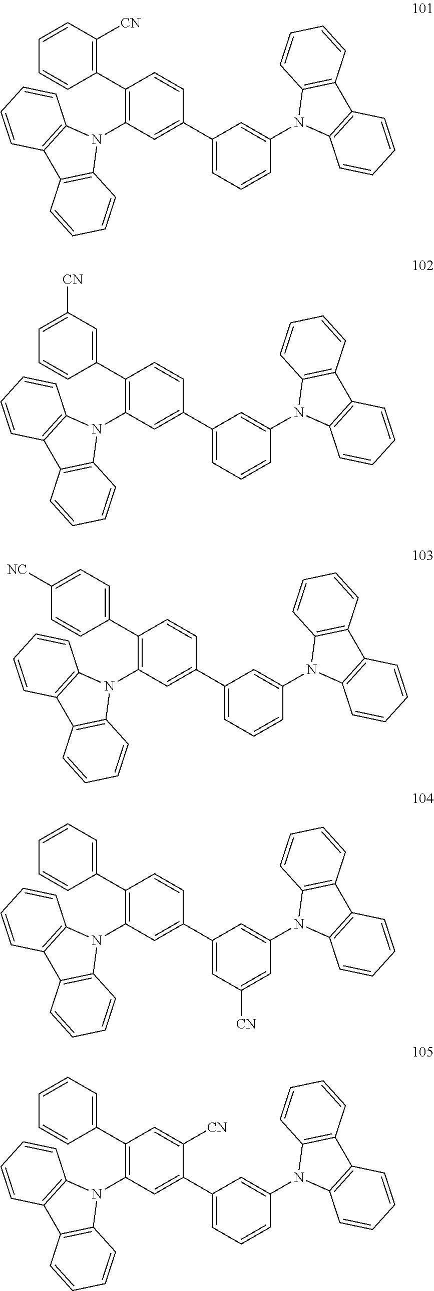

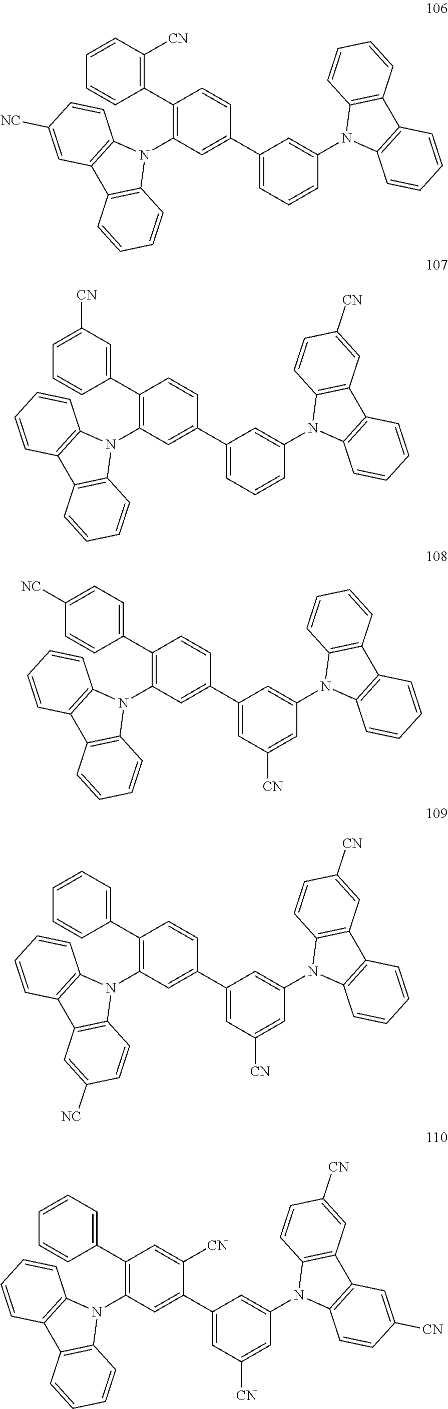

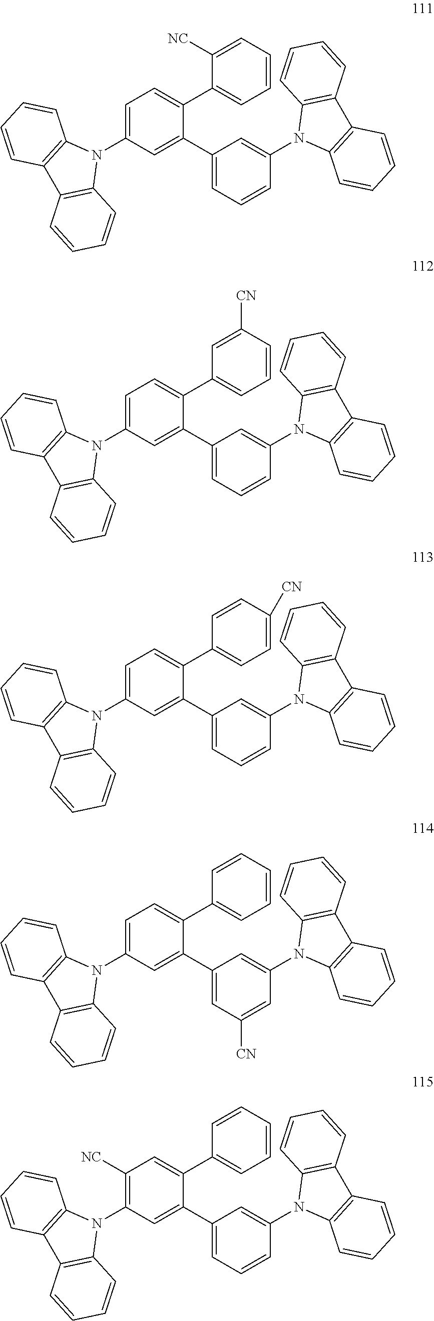 [SMILES: N#Cc1ccccc1-c1ccc(-n2c3ccccc3c3ccccc32)cc1-c1cccc(-n2c3ccccc3c3ccccc32)c1 ; N#Cc1cccc(-c2ccc(-n3c4ccccc4c4ccccc43)cc2-c2cccc(-n3c4ccccc4c4ccccc43)c2)c1 ; N#Cc1ccc(-c2ccc(-n3c4ccccc4c4ccccc43)cc2-c2cccc(-n3c4ccccc4c4ccccc43)c2)cc1 ; N#Cc1cc(-c2cc(-n3c4ccccc4c4ccccc43)ccc2-c2ccccc2)cc(-n2c3ccccc3c3ccccc32)c1 ; N#Cc1cc(-c2ccccc2)c(-c2cccc(-n3c4ccccc4c4ccccc43)c2)cc1-n1c2ccccc2c2ccccc21]

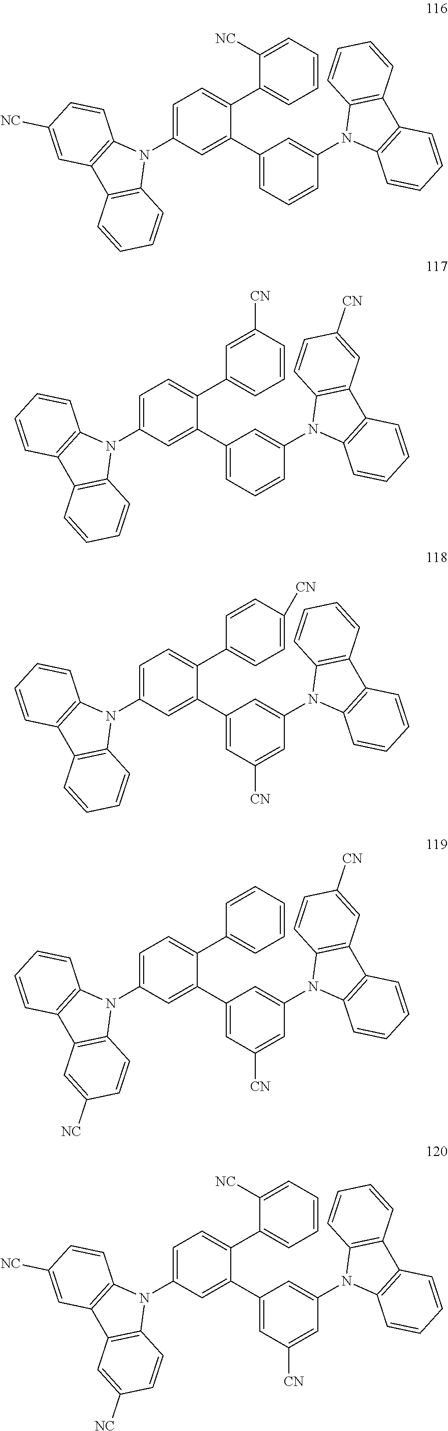 [SMILES: N#Cc1ccc2c(c1)c1ccccc1n2-c1ccc(-c2ccccc2C#N)c(-c2cccc(-n3c4ccccc4c4ccccc43)c2)c1 ; N#Cc1cccc(-c2ccc(-n3c4ccccc4c4ccccc43)cc2-c2cccc(-n3c4ccccc4c4cc(C#N)ccc43)c2)c1 ; N#Cc1ccc(-c2ccc(-n3c4ccccc4c4ccccc43)cc2-c2cc(C#N)cc(-n3c4ccccc4c4ccccc43)c2)cc1 ; N#Cc1cc(-c2cc(-n3c4ccccc4c4cc(C#N)ccc43)ccc2-c2ccccc2)cc(-n2c3ccccc3c3cc(C#N)ccc32)c1 ; N#Cc1cc(-c2cc(-n3c4ccc(C#N)cc4c4cc(C#N)ccc43)ccc2-c2ccccc2C#N)cc(-n2c3ccccc3c3ccccc32)c1]

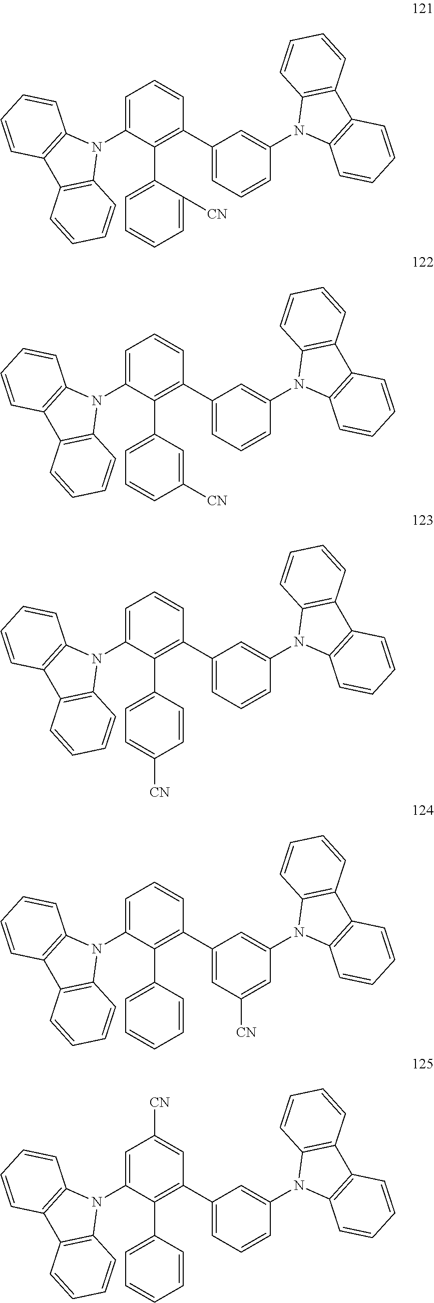 [SMILES: N#Cc1ccccc1-c1c(-c2cccc(-n3c4ccccc4c4ccccc43)c2)cccc1-n1c2ccccc2c2ccccc21 ; N#Cc1cccc(-c2c(-c3cccc(-n4c5ccccc5c5ccccc54)c3)cccc2-n2c3ccccc3c3ccccc32)c1 ; N#Cc1ccc(-c2c(-c3cccc(-n4c5ccccc5c5ccccc54)c3)cccc2-n2c3ccccc3c3ccccc32)cc1 ; N#Cc1cc(-c2cccc(-n3c4ccccc4c4ccccc43)c2-c2ccccc2)cc(-n2c3ccccc3c3ccccc32)c1 ; N#Cc1cc(-c2cccc(-n3c4ccccc4c4ccccc43)c2)c(-c2ccccc2)c(-n2c3ccccc3c3ccccc32)c1]

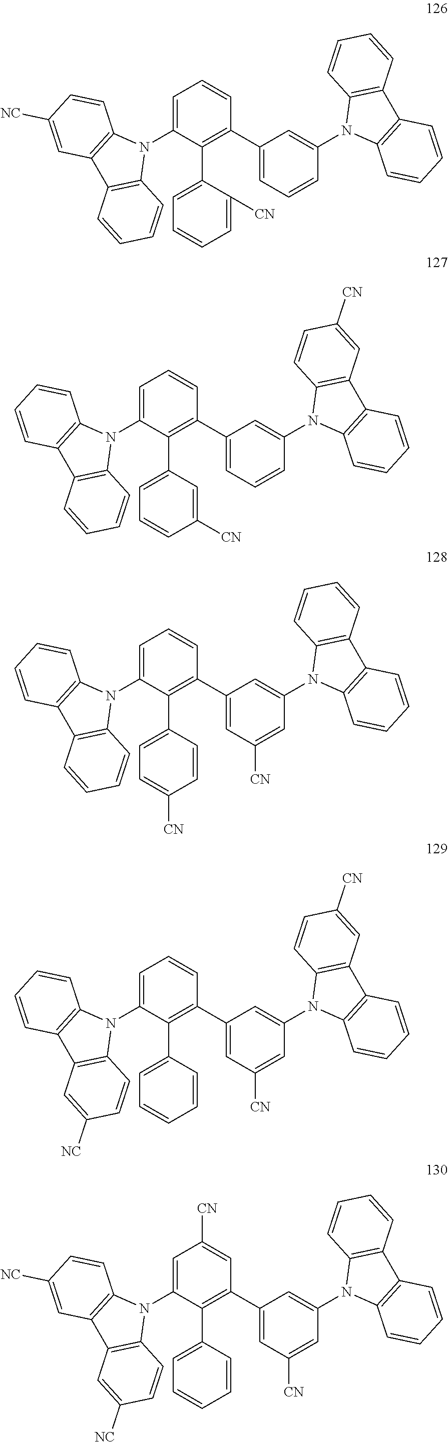 [SMILES: N#Cc1ccc2c(c1)c1ccccc1n2-c1cccc(-c2cccc(-n3c4ccccc4c4ccccc43)c2)c1-c1ccccc1C#N ; N#Cc1cccc(-c2c(-c3cccc(-n4c5ccccc5c5cc(C#N)ccc54)c3)cccc2-n2c3ccccc3c3ccccc32)c1 ; N#Cc1ccc(-c2c(-c3cc(C#N)cc(-n4c5ccccc5c5ccccc54)c3)cccc2-n2c3ccccc3c3ccccc32)cc1 ; N#Cc1cc(-c2cccc(-n3c4ccccc4c4cc(C#N)ccc43)c2-c2ccccc2)cc(-n2c3ccccc3c3cc(C#N)ccc32)c1 ; N#Cc1cc(-c2cc(C#N)cc(-n3c4ccc(C#N)cc4c4cc(C#N)ccc43)c2-c2ccccc2)cc(-n2c3ccccc3c3ccccc32)c1]

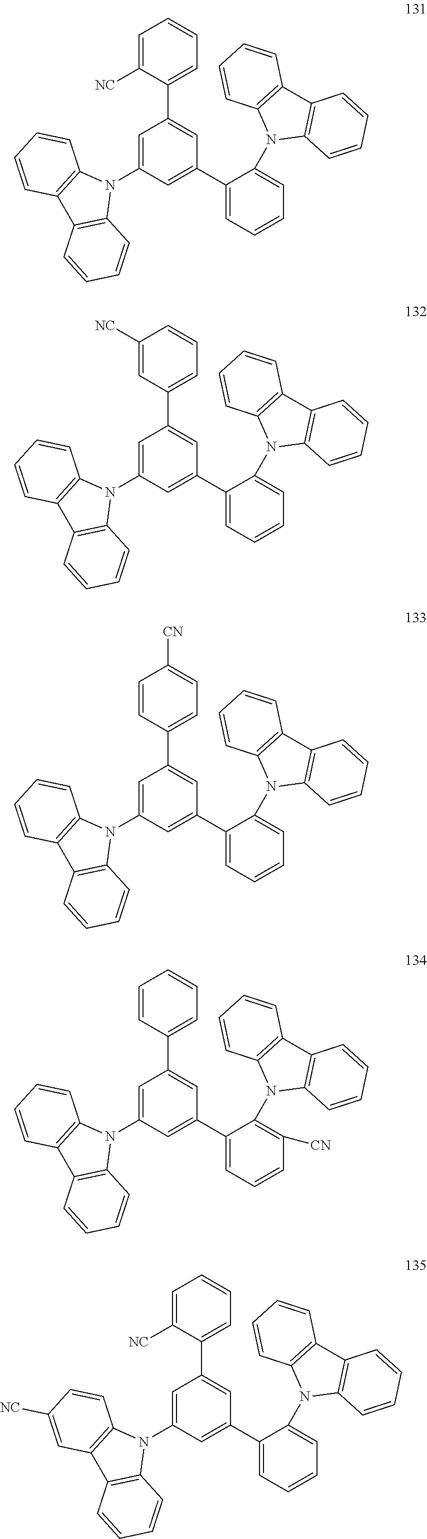 [SMILES: N#Cc1ccccc1-c1cc(-c2ccccc2-n2c3ccccc3c3ccccc32)cc(-n2c3ccccc3c3ccccc32)c1 ; N#Cc1cccc(-c2cc(-c3ccccc3-n3c4ccccc4c4ccccc43)cc(-n3c4ccccc4c4ccccc43)c2)c1 ; N#Cc1ccc(-c2cc(-c3ccccc3-n3c4ccccc4c4ccccc43)cc(-n3c4ccccc4c4ccccc43)c2)cc1 ; N#Cc1cccc(-c2cc(-c3ccccc3)cc(-n3c4ccccc4c4ccccc43)c2)c1-n1c2ccccc2c2ccccc21 ; N#Cc1ccc2c(c1)c1ccccc1n2-c1cc(-c2ccccc2C#N)cc(-c2ccccc2-n2c3ccccc3c3ccccc32)c1]

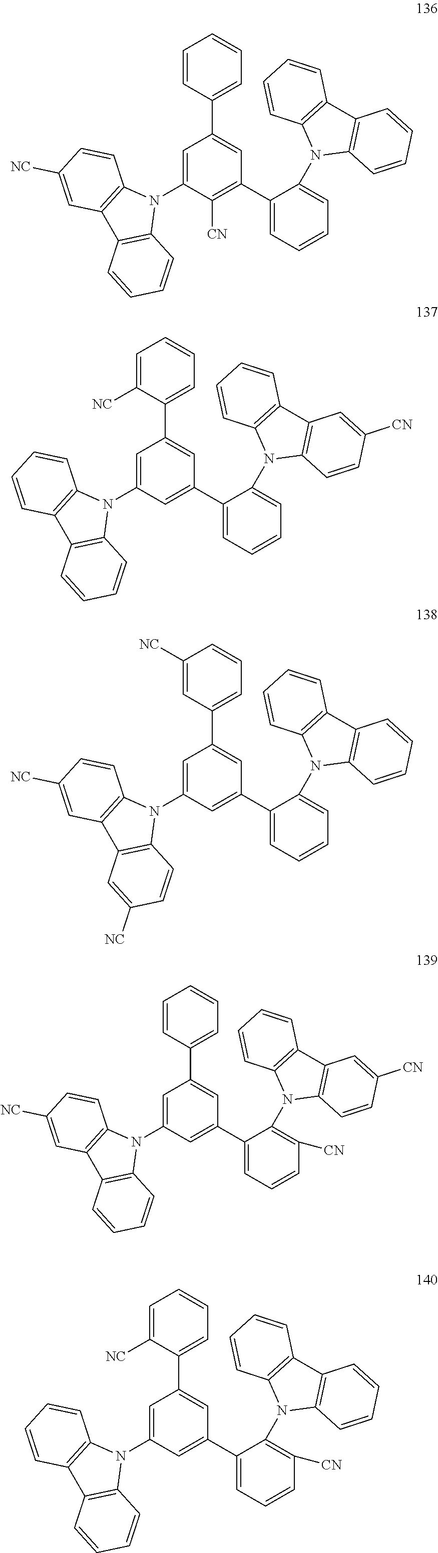 [SMILES: N#Cc1ccc2c(c1)c1ccccc1n2-c1cc(-c2ccccc2)cc(-c2ccccc2-n2c3ccccc3c3ccccc32)c1C#N ; N#Cc1ccc2c(c1)c1ccccc1n2-c1ccccc1-c1cc(-c2ccccc2C#N)cc(-n2c3ccccc3c3ccccc32)c1 ; N#Cc1cccc(-c2cc(-c3ccccc3-n3c4ccccc4c4ccccc43)cc(-n3c4ccc(C#N)cc4c4cc(C#N)ccc43)c2)c1 ; N#Cc1ccc2c(c1)c1ccccc1n2-c1cc(-c2ccccc2)cc(-c2cccc(C#N)c2-n2c3ccccc3c3cc(C#N)ccc32)c1 ; N#Cc1ccccc1-c1cc(-c2cccc(C#N)c2-n2c3ccccc3c3ccccc32)cc(-n2c3ccccc3c3ccccc32)c1]

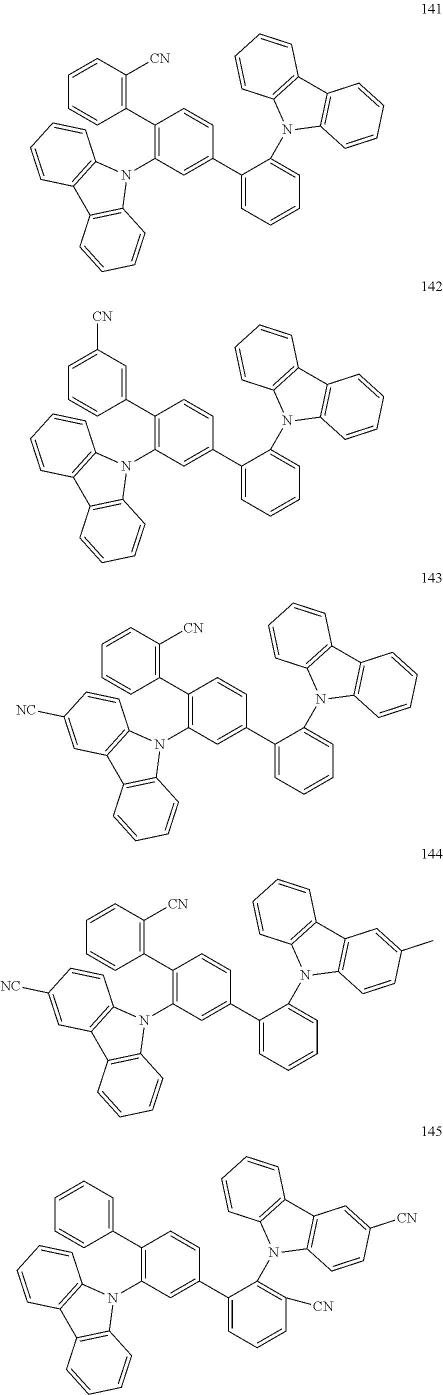

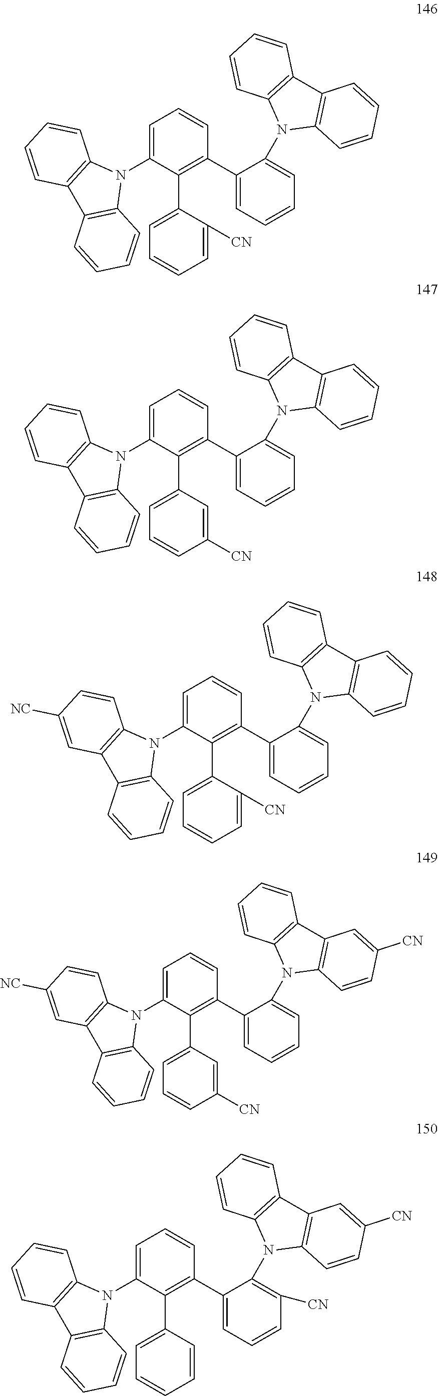

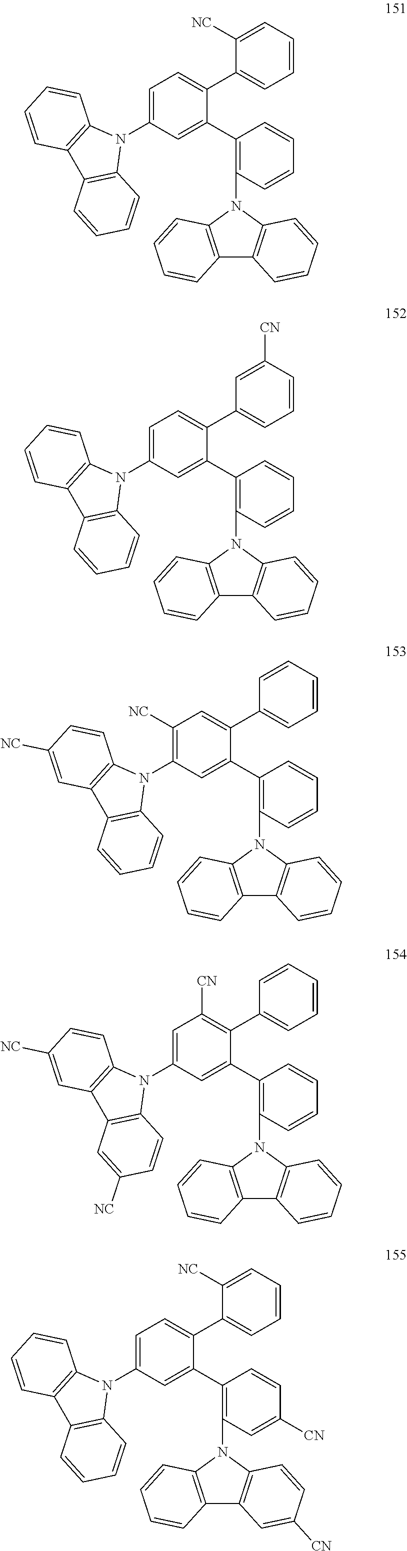

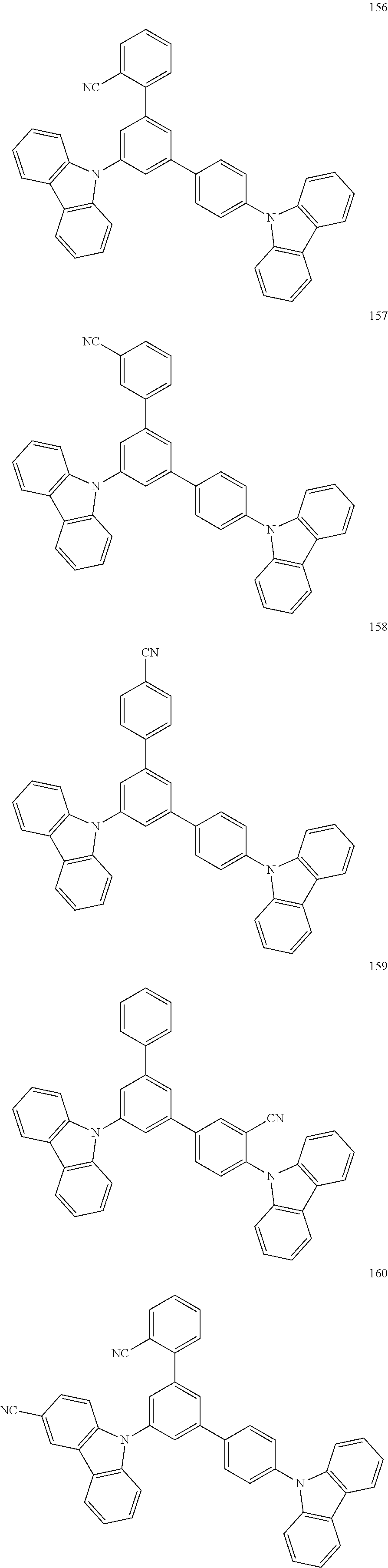 [SMILES: N#Cc1ccccc1-c1cc(-c2ccc(-n3c4ccccc4c4ccccc43)cc2)cc(-n2c3ccccc3c3ccccc32)c1 ; N#Cc1cccc(-c2cc(-c3ccc(-n4c5ccccc5c5ccccc54)cc3)cc(-n3c4ccccc4c4ccccc43)c2)c1 ; N#Cc1ccc(-c2cc(-c3ccc(-n4c5ccccc5c5ccccc54)cc3)cc(-n3c4ccccc4c4ccccc43)c2)cc1 ; N#Cc1cc(-c2cc(-c3ccccc3)cc(-n3c4ccccc4c4ccccc43)c2)ccc1-n1c2ccccc2c2ccccc21 ; N#Cc1ccc2c(c1)c1ccccc1n2-c1cc(-c2ccc(-n3c4ccccc4c4ccccc43)cc2)cc(-c2ccccc2C#N)c1]

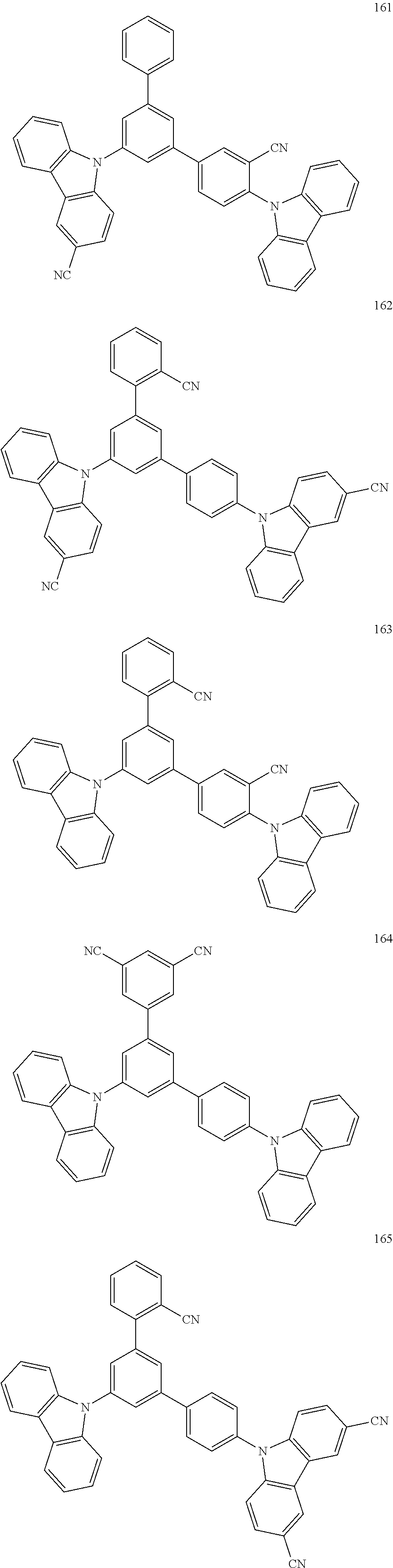 [SMILES: N#Cc1ccc2c(c1)c1ccccc1n2-c1cc(-c2ccccc2)cc(-c2ccc(-n3c4ccccc4c4ccccc43)c(C#N)c2)c1 ; N#Cc1ccc2c(c1)c1ccccc1n2-c1ccc(-c2cc(-c3ccccc3C#N)cc(-n3c4ccccc4c4cc(C#N)ccc43)c2)cc1 ; N#Cc1ccccc1-c1cc(-c2ccc(-n3c4ccccc4c4ccccc43)c(C#N)c2)cc(-n2c3ccccc3c3ccccc32)c1 ; N#Cc1cc(C#N)cc(-c2cc(-c3ccc(-n4c5ccccc5c5ccccc54)cc3)cc(-n3c4ccccc4c4ccccc43)c2)c1 ; N#Cc1ccc2c(c1)c1cc(C#N)ccc1n2-c1ccc(-c2cc(-c3ccccc3C#N)cc(-n3c4ccccc4c4ccccc43)c2)cc1]

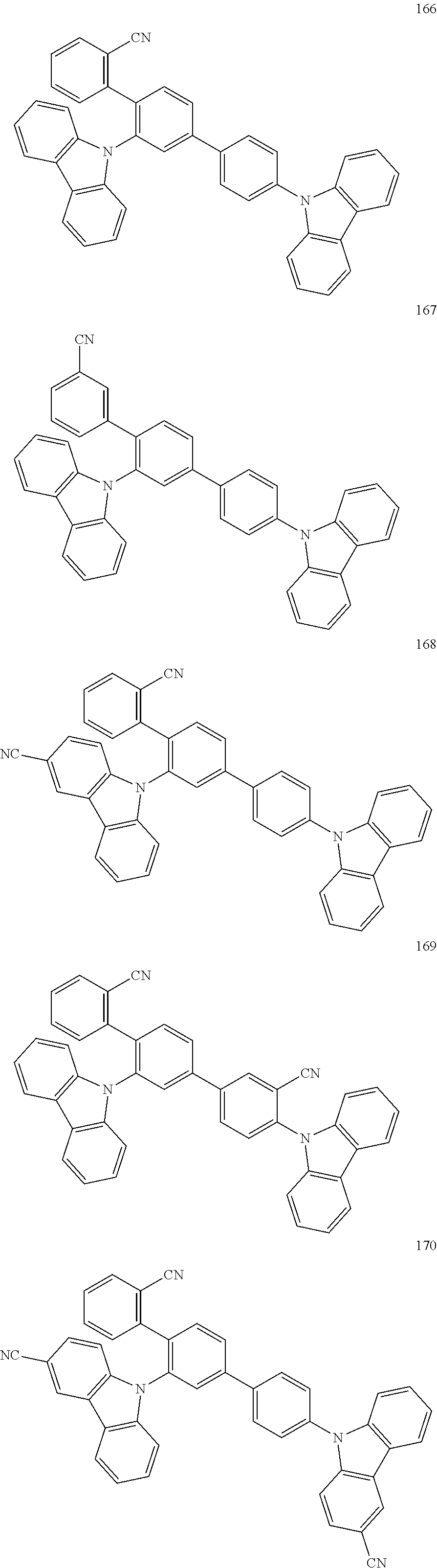 [SMILES: N#Cc1ccccc1-c1ccc(-c2ccc(-n3c4ccccc4c4ccccc43)cc2)cc1-n1c2ccccc2c2ccccc21 ; N#Cc1cccc(-c2ccc(-c3ccc(-n4c5ccccc5c5ccccc54)cc3)cc2-n2c3ccccc3c3ccccc32)c1 ; N#Cc1ccc2c(c1)c1ccccc1n2-c1cc(-c2ccc(-n3c4ccccc4c4ccccc43)cc2)ccc1-c1ccccc1C#N ; N#Cc1ccccc1-c1ccc(-c2ccc(-n3c4ccccc4c4ccccc43)c(C#N)c2)cc1-n1c2ccccc2c2ccccc21 ; N#Cc1ccc2c(c1)c1ccccc1n2-c1ccc(-c2ccc(-c3ccccc3C#N)c(-n3c4ccccc4c4cc(C#N)ccc43)c2)cc1]

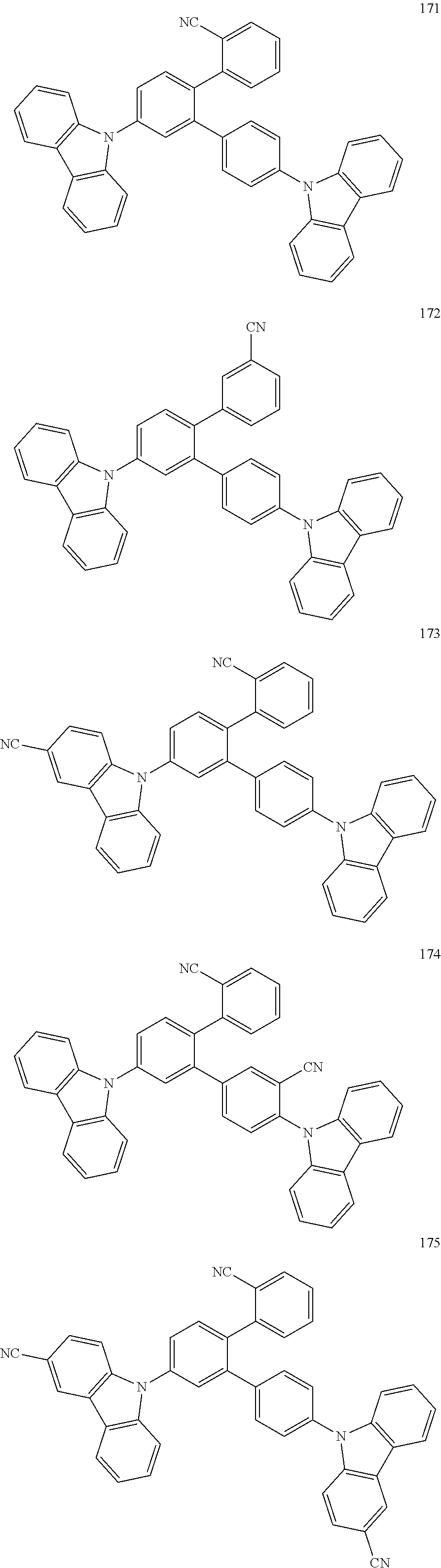 [SMILES: N#Cc1ccccc1-c1ccc(-n2c3ccccc3c3ccccc32)cc1-c1ccc(-n2c3ccccc3c3ccccc32)cc1 ; N#Cc1cccc(-c2ccc(-n3c4ccccc4c4ccccc43)cc2-c2ccc(-n3c4ccccc4c4ccccc43)cc2)c1 ; N#Cc1ccc2c(c1)c1ccccc1n2-c1ccc(-c2ccccc2C#N)c(-c2ccc(-n3c4ccccc4c4ccccc43)cc2)c1 ; N#Cc1ccccc1-c1ccc(-n2c3ccccc3c3ccccc32)cc1-c1ccc(-n2c3ccccc3c3ccccc32)c(C#N)c1 ; N#Cc1ccc2c(c1)c1ccccc1n2-c1ccc(-c2cc(-n3c4ccccc4c4cc(C#N)ccc43)ccc2-c2ccccc2C#N)cc1]

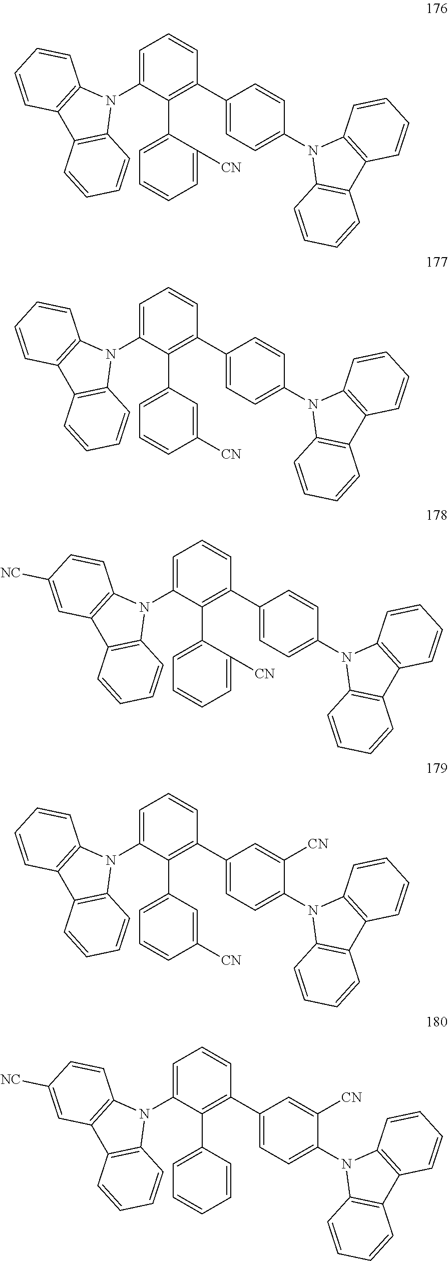 [SMILES: N#Cc1ccccc1-c1c(-c2ccc(-n3c4ccccc4c4ccccc43)cc2)cccc1-n1c2ccccc2c2ccccc21 ; N#Cc1cccc(-c2c(-c3ccc(-n4c5ccccc5c5ccccc54)cc3)cccc2-n2c3ccccc3c3ccccc32)c1 ; N#Cc1ccc2c(c1)c1ccccc1n2-c1cccc(-c2ccc(-n3c4ccccc4c4ccccc43)cc2)c1-c1ccccc1C#N ; N#Cc1cccc(-c2c(-c3ccc(-n4c5ccccc5c5ccccc54)c(C#N)c3)cccc2-n2c3ccccc3c3ccccc32)c1 ; N#Cc1ccc2c(c1)c1ccccc1n2-c1cccc(-c2ccc(-n3c4ccccc4c4ccccc43)c(C#N)c2)c1-c1ccccc1]

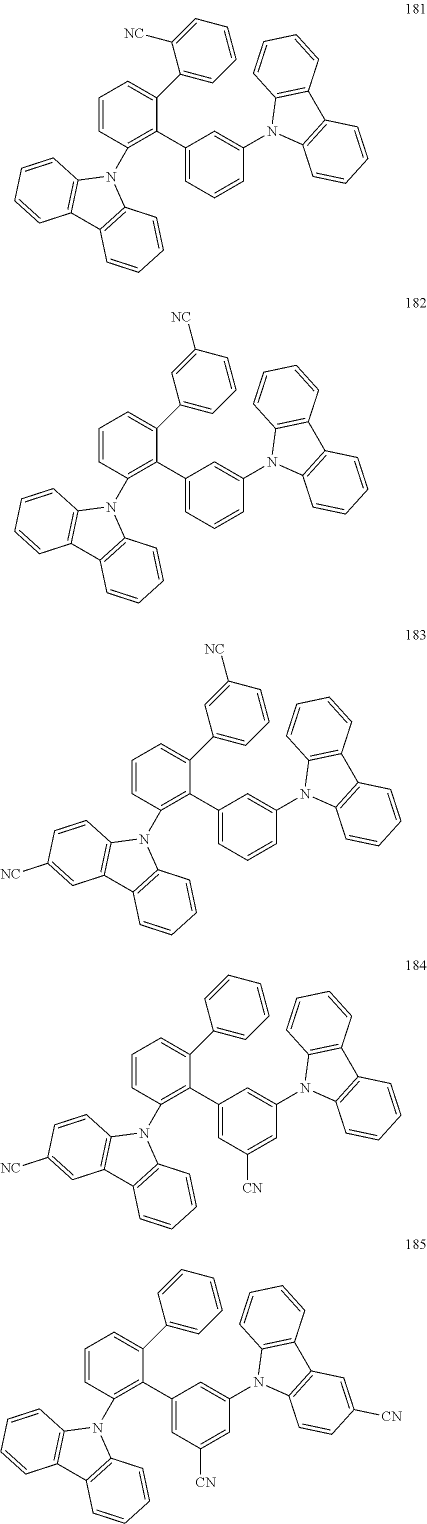

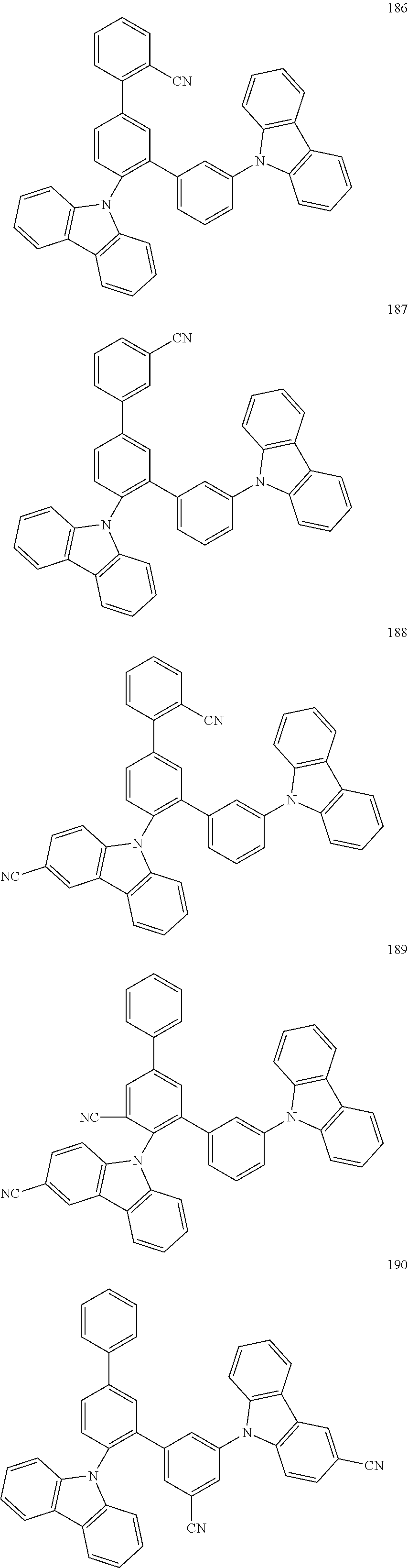

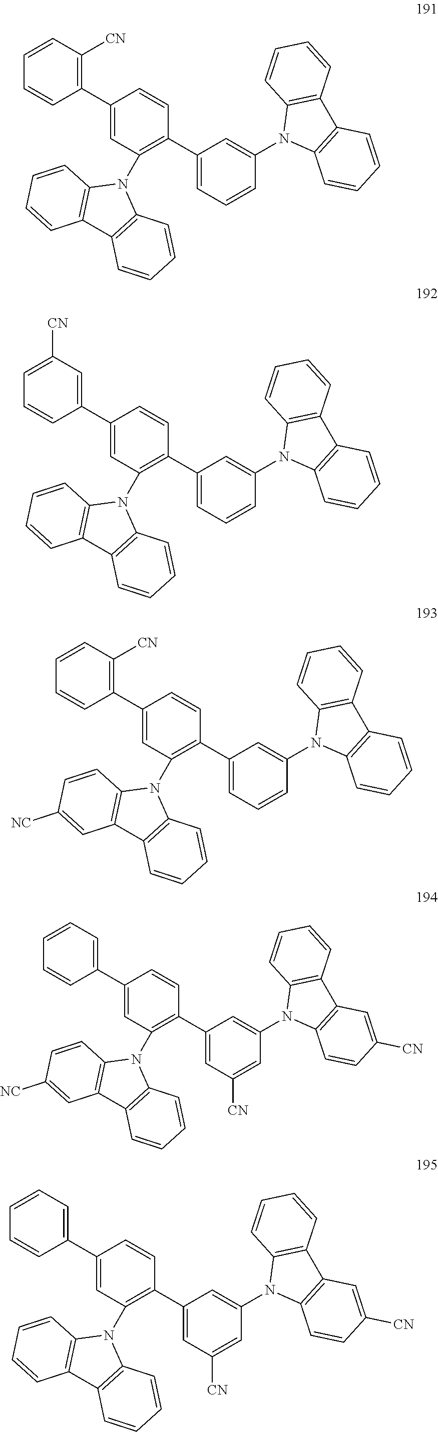

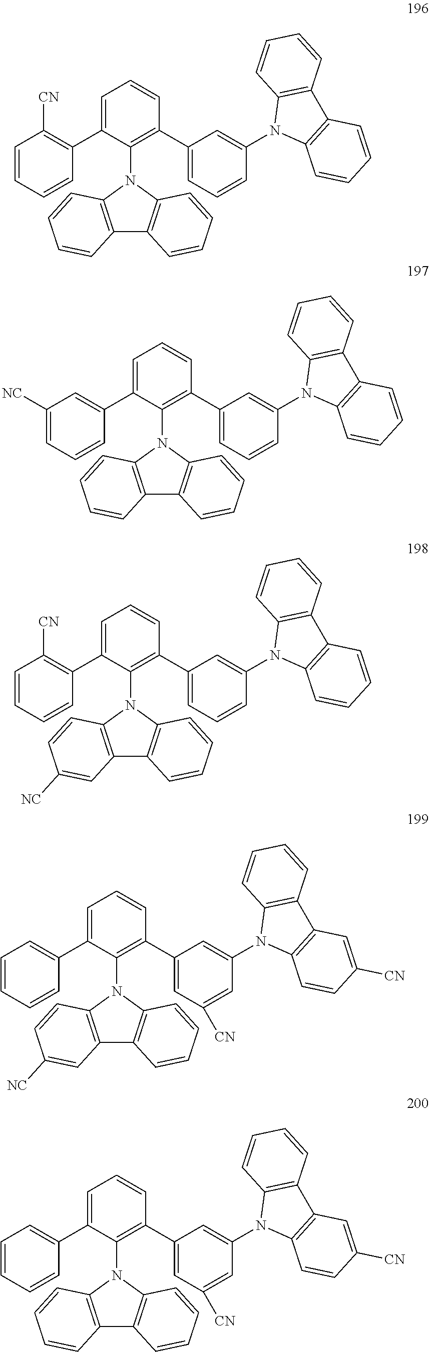 [SMILES: N#Cc1ccccc1-c1cccc(-c2cccc(-n3c4ccccc4c4ccccc43)c2)c1-n1c2ccccc2c2ccccc21 ; N#Cc1cccc(-c2cccc(-c3cccc(-n4c5ccccc5c5ccccc54)c3)c2-n2c3ccccc3c3ccccc32)c1 ; N#Cc1ccc2c(c1)c1ccccc1n2-c1c(-c2cccc(-n3c4ccccc4c4ccccc43)c2)cccc1-c1ccccc1C#N ; N#Cc1cc(-c2cccc(-c3ccccc3)c2-n2c3ccccc3c3cc(C#N)ccc32)cc(-n2c3ccccc3c3cc(C#N)ccc32)c1 ; N#Cc1cc(-c2cccc(-c3ccccc3)c2-n2c3ccccc3c3ccccc32)cc(-n2c3ccccc3c3cc(C#N)ccc32)c1]

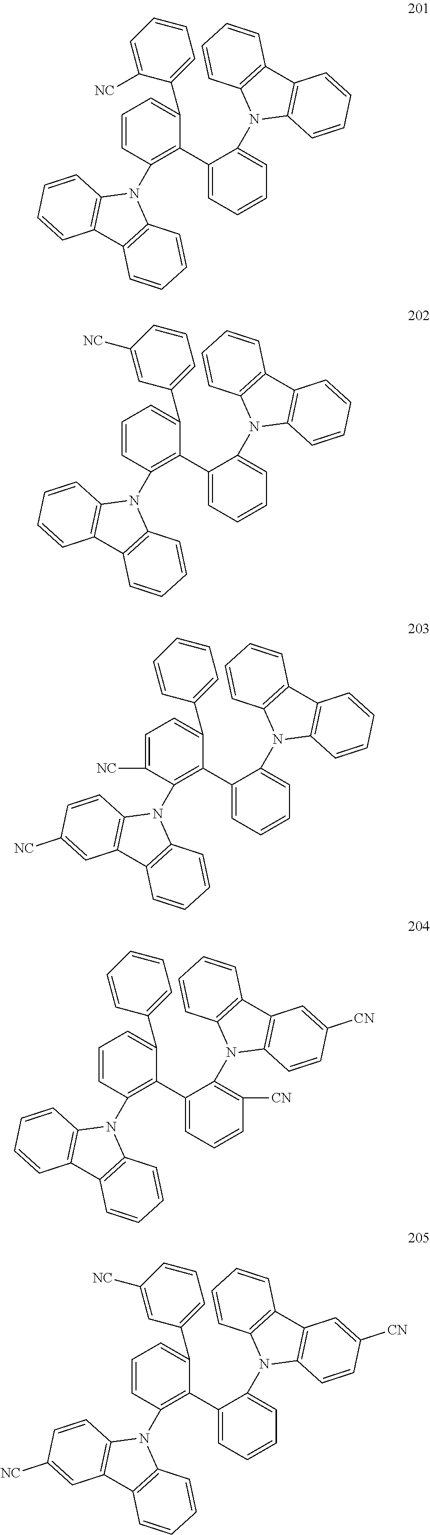

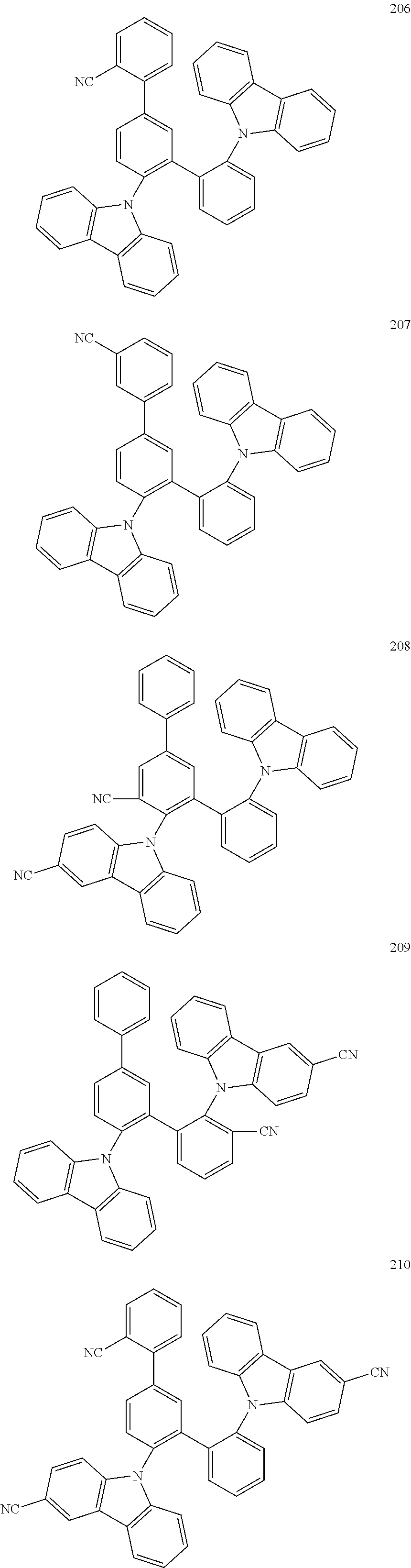

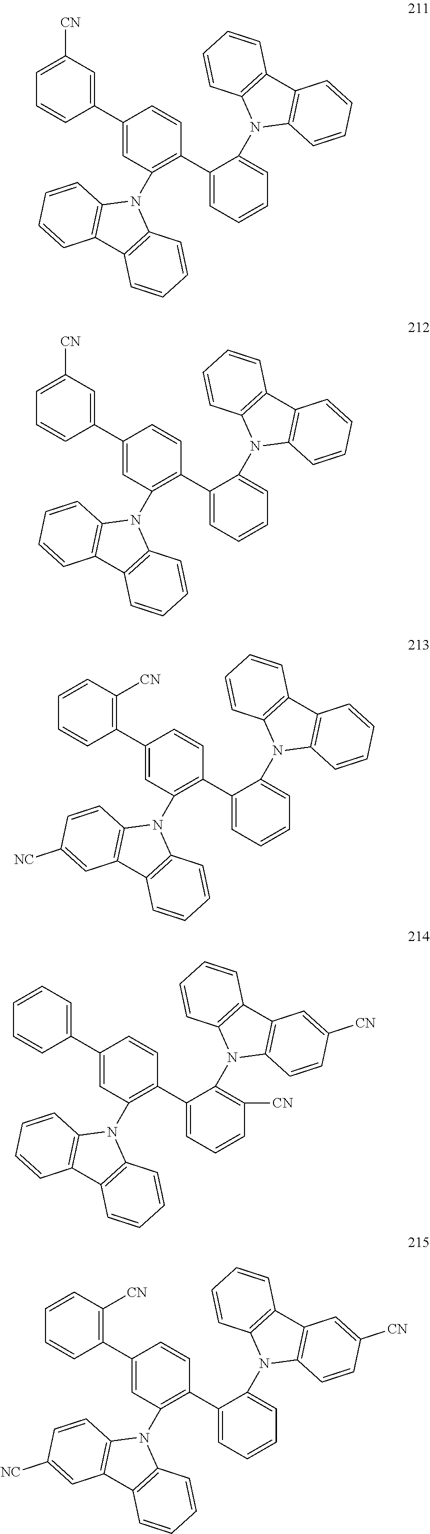

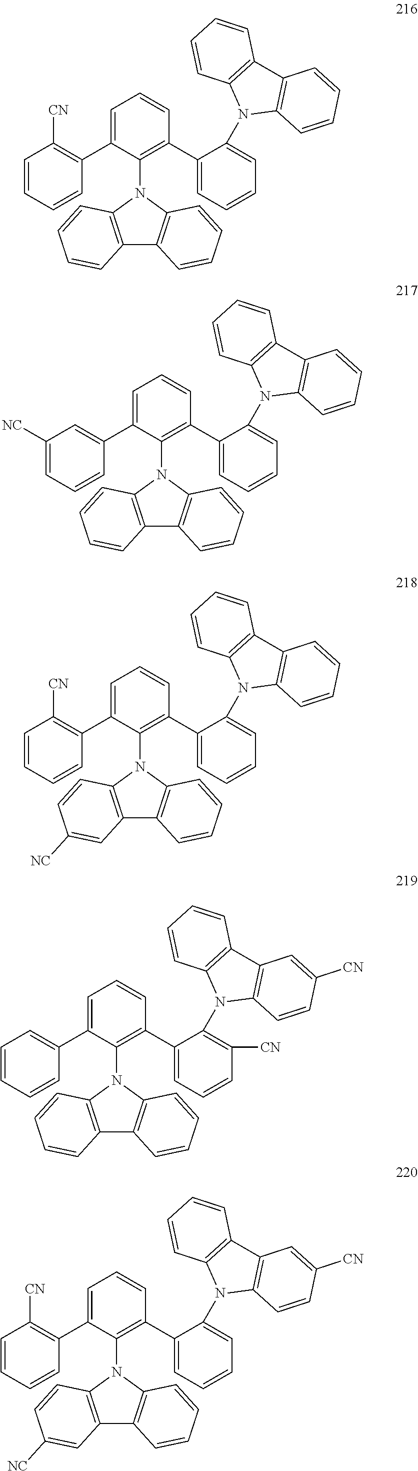

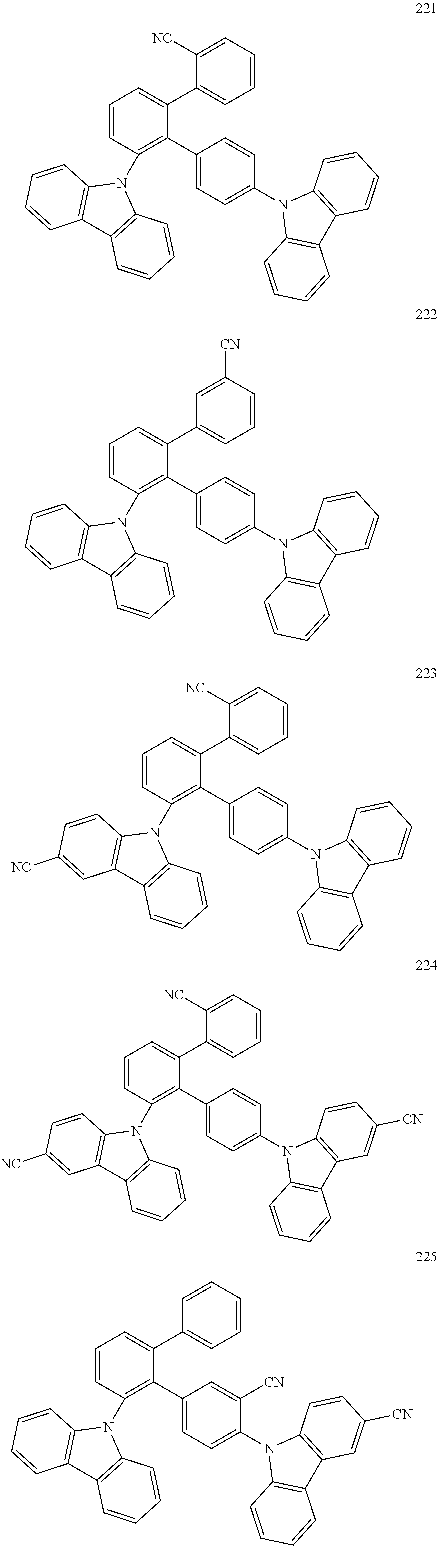 [SMILES: N#Cc1ccccc1-c1cccc(-n2c3ccccc3c3ccccc32)c1-c1ccc(-n2c3ccccc3c3ccccc32)cc1 ; N#Cc1cccc(-c2cccc(-n3c4ccccc4c4ccccc43)c2-c2ccc(-n3c4ccccc4c4ccccc43)cc2)c1 ; N#Cc1ccc2c(c1)c1ccccc1n2-c1cccc(-c2ccccc2C#N)c1-c1ccc(-n2c3ccccc3c3ccccc32)cc1 ; N#Cc1ccc2c(c1)c1ccccc1n2-c1ccc(-c2c(-c3ccccc3C#N)cccc2-n2c3ccccc3c3cc(C#N)ccc32)cc1 ; N#Cc1ccc2c(c1)c1ccccc1n2-c1ccc(-c2c(-c3ccccc3)cccc2-n2c3ccccc3c3ccccc32)cc1C#N]

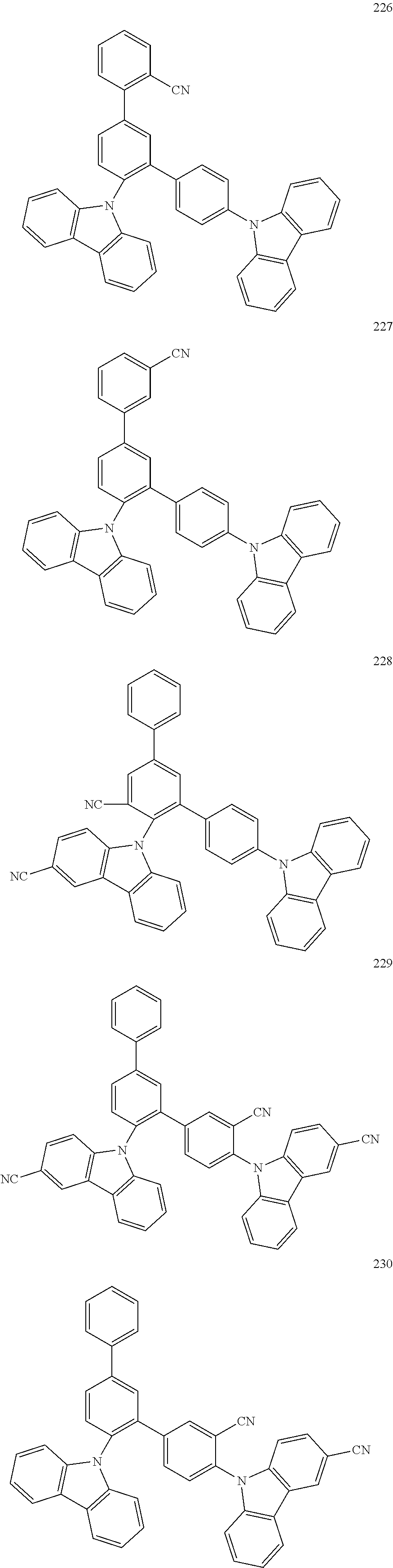 [SMILES: N#Cc1ccccc1-c1ccc(-n2c3ccccc3c3ccccc32)c(-c2ccc(-n3c4ccccc4c4ccccc43)cc2)c1 ; N#Cc1cccc(-c2ccc(-n3c4ccccc4c4ccccc43)c(-c3ccc(-n4c5ccccc5c5ccccc54)cc3)c2)c1 ; N#Cc1ccc2c(c1)c1ccccc1n2-c1c(C#N)cc(-c2ccccc2)cc1-c1ccc(-n2c3ccccc3c3ccccc32)cc1 ; N#Cc1ccc2c(c1)c1ccccc1n2-c1ccc(-c2cc(-c3ccccc3)ccc2-n2c3ccccc3c3cc(C#N)ccc32)cc1C#N ; N#Cc1ccc2c(c1)c1ccccc1n2-c1ccc(-c2cc(-c3ccccc3)ccc2-n2c3ccccc3c3ccccc32)cc1C#N]

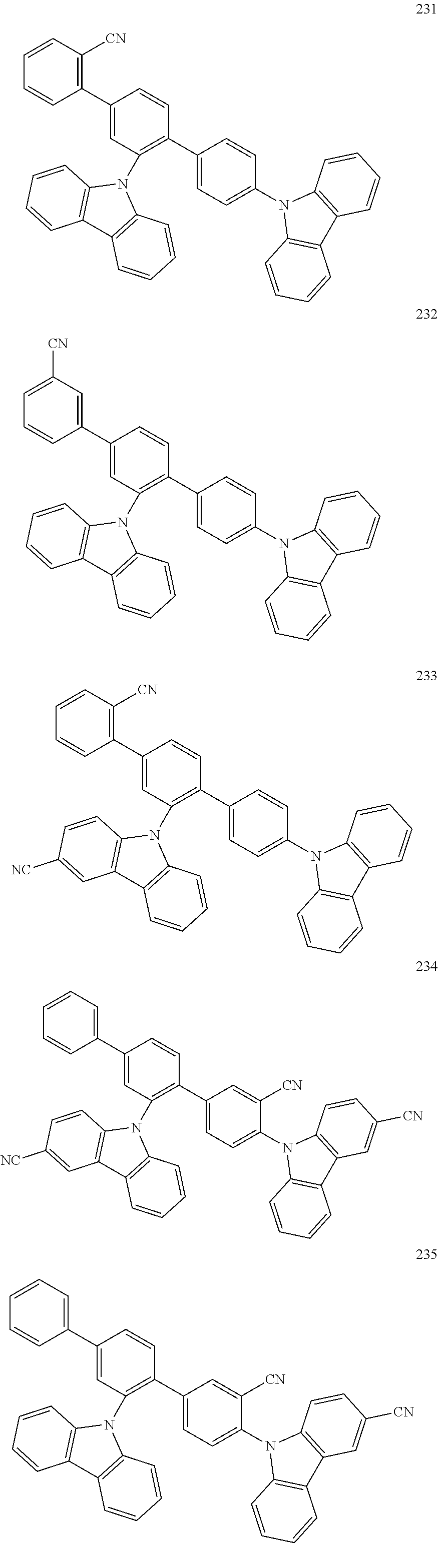 [SMILES: N#Cc1ccccc1-c1ccc(-c2ccc(-n3c4ccccc4c4ccccc43)cc2)c(-n2c3ccccc3c3ccccc32)c1 ; N#Cc1cccc(-c2ccc(-c3ccc(-n4c5ccccc5c5ccccc54)cc3)c(-n3c4ccccc4c4ccccc43)c2)c1 ; N#Cc1ccc2c(c1)c1ccccc1n2-c1cc(-c2ccccc2C#N)ccc1-c1ccc(-n2c3ccccc3c3ccccc32)cc1 ; N#Cc1ccc2c(c1)c1ccccc1n2-c1ccc(-c2ccc(-c3ccccc3)cc2-n2c3ccccc3c3cc(C#N)ccc32)cc1C#N ; N#Cc1ccc2c(c1)c1ccccc1n2-c1ccc(-c2ccc(-c3ccccc3)cc2-n2c3ccccc3c3ccccc32)cc1C#N]

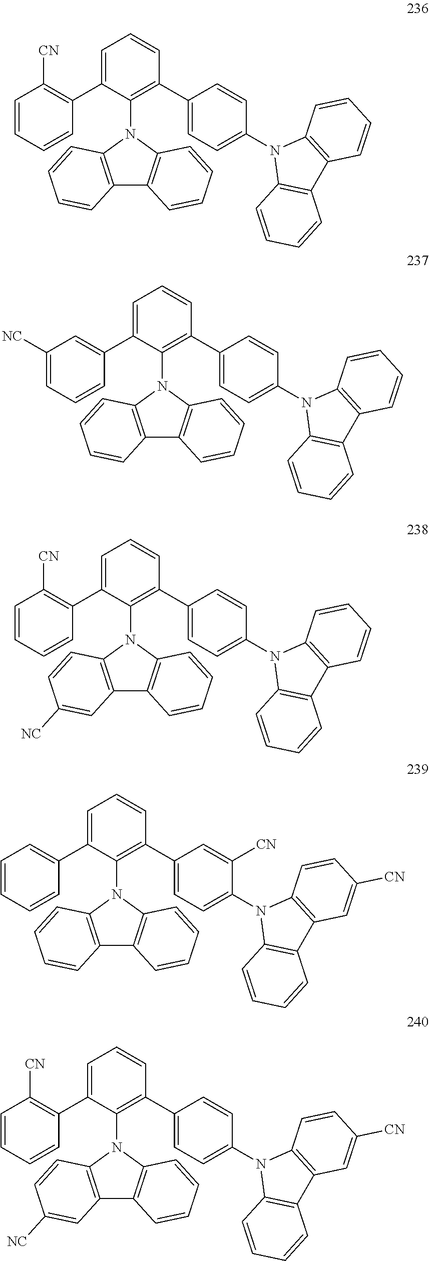 [SMILES: N#Cc1ccccc1-c1cccc(-c2ccc(-n3c4ccccc4c4ccccc43)cc2)c1-n1c2ccccc2c2ccccc21 ; N#Cc1cccc(-c2cccc(-c3ccc(-n4c5ccccc5c5ccccc54)cc3)c2-n2c3ccccc3c3ccccc32)c1 ; N#Cc1ccc2c(c1)c1ccccc1n2-c1c(-c2ccc(-n3c4ccccc4c4ccccc43)cc2)cccc1-c1ccccc1C#N ; N#Cc1ccc2c(c1)c1ccccc1n2-c1ccc(-c2cccc(-c3ccccc3)c2-n2c3ccccc3c3ccccc32)cc1C#N ; N#Cc1ccc2c(c1)c1ccccc1n2-c1ccc(-c2cccc(-c3ccccc3C#N)c2-n2c3ccccc3c3cc(C#N)ccc32)cc1]

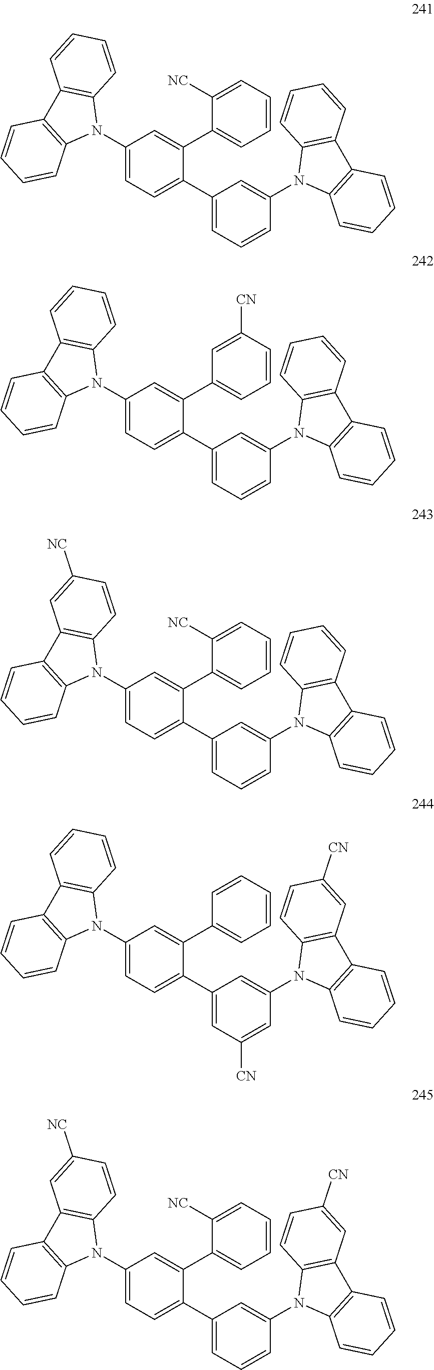 [SMILES: N#Cc1ccccc1-c1cc(-n2c3ccccc3c3ccccc32)ccc1-c1cccc(-n2c3ccccc3c3ccccc32)c1 ; N#Cc1cccc(-c2cc(-n3c4ccccc4c4ccccc43)ccc2-c2cccc(-n3c4ccccc4c4ccccc43)c2)c1 ; N#Cc1ccc2c(c1)c1ccccc1n2-c1ccc(-c2cccc(-n3c4ccccc4c4ccccc43)c2)c(-c2ccccc2C#N)c1 ; N#Cc1cc(-c2ccc(-n3c4ccccc4c4ccccc43)cc2-c2ccccc2)cc(-n2c3ccccc3c3cc(C#N)ccc32)c1 ; N#Cc1ccc2c(c1)c1ccccc1n2-c1cccc(-c2ccc(-n3c4ccccc4c4cc(C#N)ccc43)cc2-c2ccccc2C#N)c1]

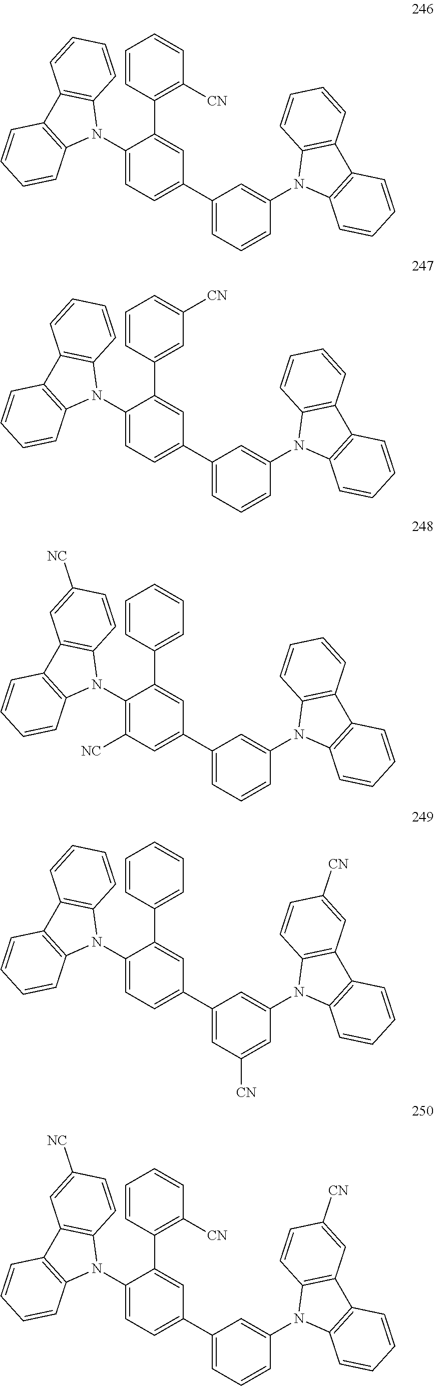 [SMILES: N#Cc1ccccc1-c1cc(-c2cccc(-n3c4ccccc4c4ccccc43)c2)ccc1-n1c2ccccc2c2ccccc21 ; N#Cc1cccc(-c2cc(-c3cccc(-n4c5ccccc5c5ccccc54)c3)ccc2-n2c3ccccc3c3ccccc32)c1 ; N#Cc1ccc2c(c1)c1ccccc1n2-c1c(C#N)cc(-c2cccc(-n3c4ccccc4c4ccccc43)c2)cc1-c1ccccc1 ; N#Cc1cc(-c2ccc(-n3c4ccccc4c4ccccc43)c(-c3ccccc3)c2)cc(-n2c3ccccc3c3cc(C#N)ccc32)c1 ; N#Cc1ccc2c(c1)c1ccccc1n2-c1cccc(-c2ccc(-n3c4ccccc4c4cc(C#N)ccc43)c(-c3ccccc3C#N)c2)c1]

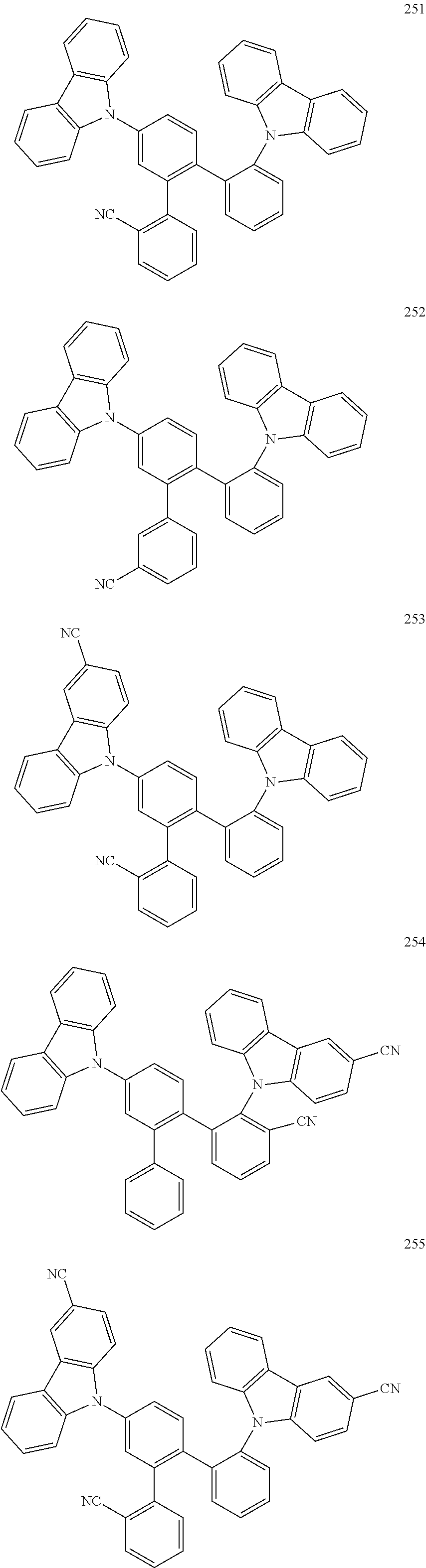 [SMILES: N#Cc1ccccc1-c1cc(-n2c3ccccc3c3ccccc32)ccc1-c1ccccc1-n1c2ccccc2c2ccccc21 ; N#Cc1cccc(-c2cc(-n3c4ccccc4c4ccccc43)ccc2-c2ccccc2-n2c3ccccc3c3ccccc32)c1 ; N#Cc1ccc2c(c1)c1ccccc1n2-c1ccc(-c2ccccc2-n2c3ccccc3c3ccccc32)c(-c2ccccc2C#N)c1 ; N#Cc1ccc2c(c1)c1ccccc1n2-c1c(C#N)cccc1-c1ccc(-n2c3ccccc3c3ccccc32)cc1-c1ccccc1 ; N#Cc1ccc2c(c1)c1ccccc1n2-c1ccc(-c2ccccc2-n2c3ccccc3c3cc(C#N)ccc32)c(-c2ccccc2C#N)c1]

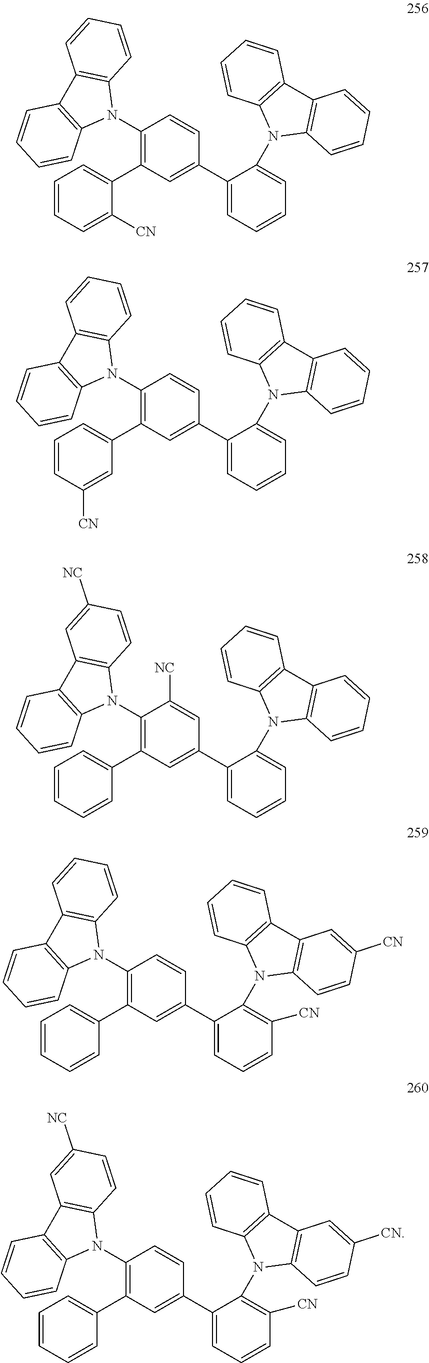

In some embodiments, the electron transporting host may include DPEPO and/or TSPO1:

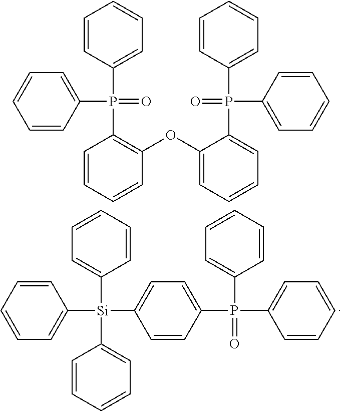

In some embodiments, the hole transporting host may be Group HH1, but embodiments are not limited thereto:

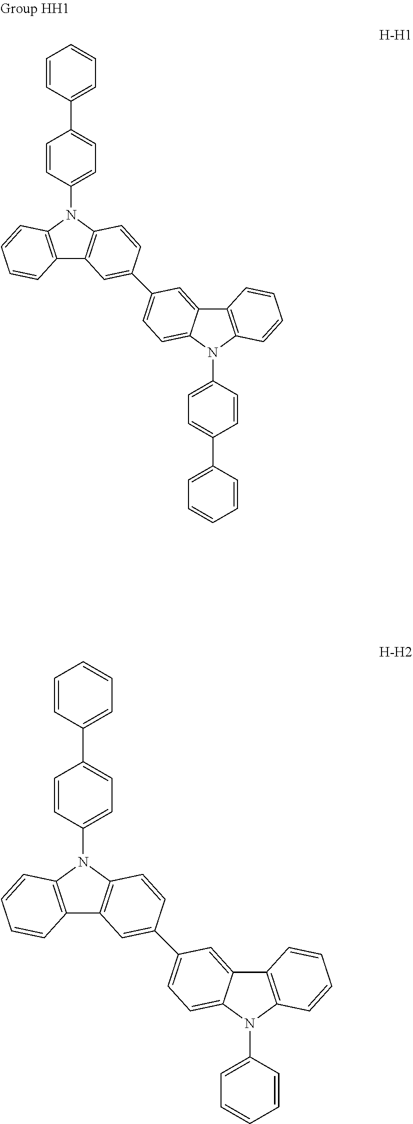 [SMILES: c1ccc(-c2ccc(-n3c4ccccc4c4cc(-c5ccc6c(c5)c5ccccc5n6-c5ccc(-c6ccccc6)cc5)ccc43)cc2)cc1 ; c1ccc(-c2ccc(-n3c4ccccc4c4cc(-c5ccc6c(c5)c5ccccc5n6-c5ccccc5)ccc43)cc2)cc1]

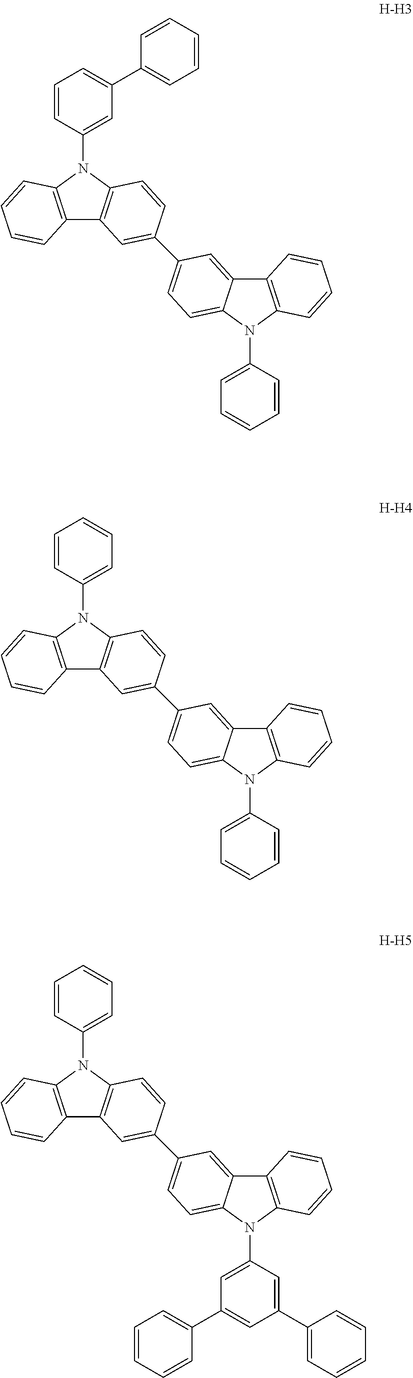 [SMILES: c1ccc(-c2cccc(-n3c4ccccc4c4cc(-c5ccc6c(c5)c5ccccc5n6-c5ccccc5)ccc43)c2)cc1 ; c1ccc(-n2c3ccccc3c3cc(-c4ccc5c(c4)c4ccccc4n5-c4ccccc4)ccc32)cc1 ; c1ccc(-c2cc(-c3ccccc3)cc(-n3c4ccccc4c4cc(-c5ccc6c(c5)c5ccccc5n6-c5ccccc5)ccc43)c2)cc1]

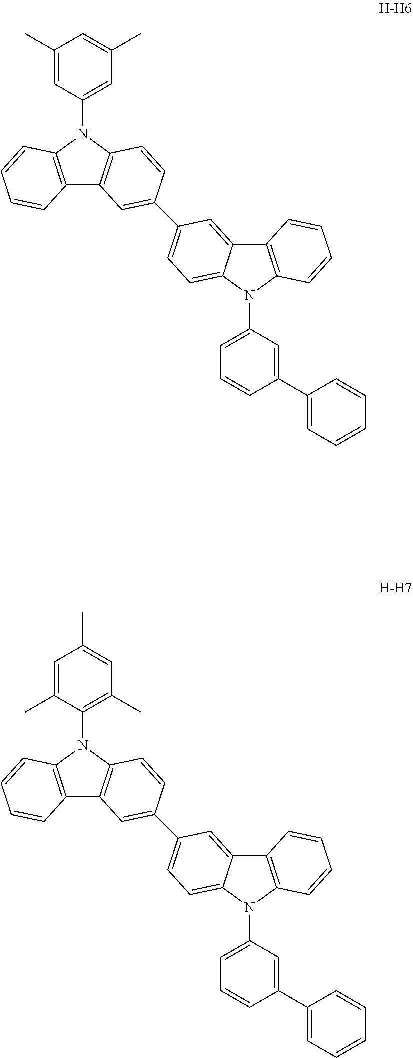 [SMILES: Cc1cc(C)cc(-n2c3ccccc3c3cc(-c4ccc5c(c4)c4ccccc4n5-c4cccc(-c5ccccc5)c4)ccc32)c1 ; Cc1cc(C)c(-n2c3ccccc3c3cc(-c4ccc5c(c4)c4ccccc4n5-c4cccc(-c5ccccc5)c4)ccc32)c(C)c1]

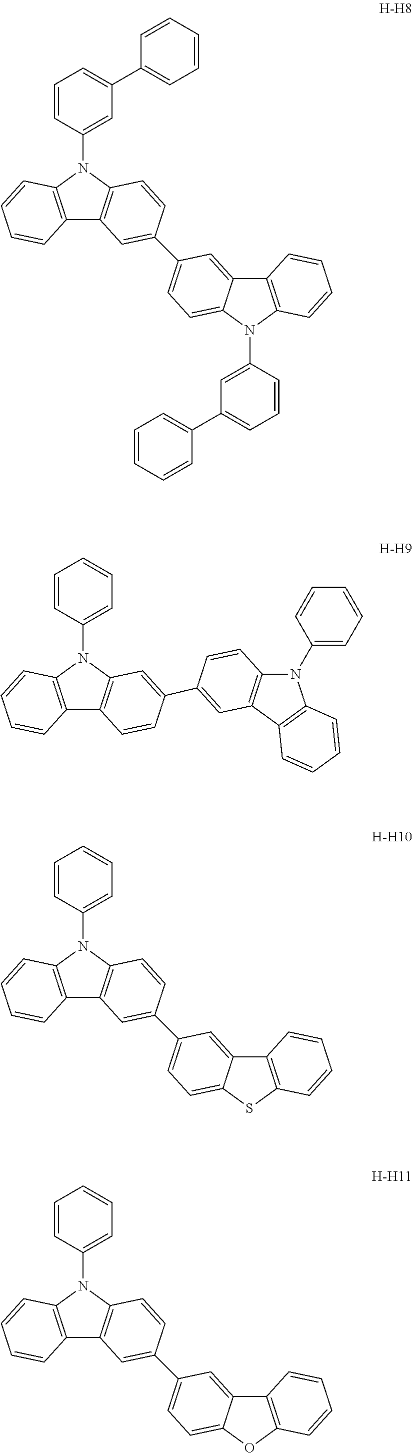 [SMILES: c1ccc(-c2cccc(-n3c4ccccc4c4cc(-c5ccc6c(c5)c5ccccc5n6-c5cccc(-c6ccccc6)c5)ccc43)c2)cc1 ; c1ccc(-n2c3ccccc3c3cc(-c4ccc5c6ccccc6n(-c6ccccc6)c5c4)ccc32)cc1 ; c1ccc(-n2c3ccccc3c3cc(-c4ccc5sc6ccccc6c5c4)ccc32)cc1 ; c1ccc(-n2c3ccccc3c3cc(-c4ccc5oc6ccccc6c5c4)ccc32)cc1]

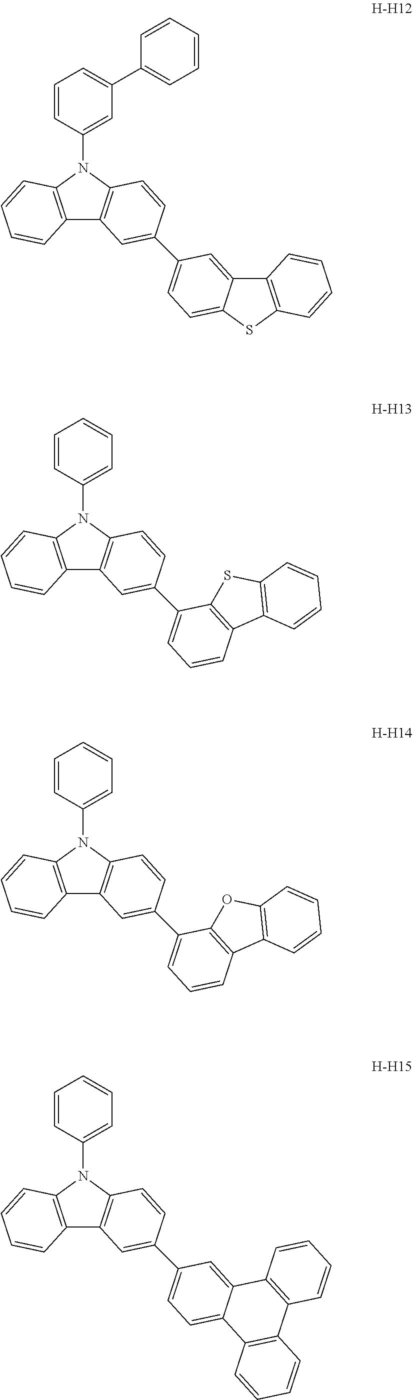 [SMILES: c1ccc(-c2cccc(-n3c4ccccc4c4cc(-c5ccc6sc7ccccc7c6c5)ccc43)c2)cc1 ; c1ccc(-n2c3ccccc3c3cc(-c4cccc5c4sc4ccccc45)ccc32)cc1 ; c1ccc(-n2c3ccccc3c3cc(-c4cccc5c4oc4ccccc45)ccc32)cc1 ; c1ccc(-n2c3ccccc3c3cc(-c4ccc5c6ccccc6c6ccccc6c5c4)ccc32)cc1]

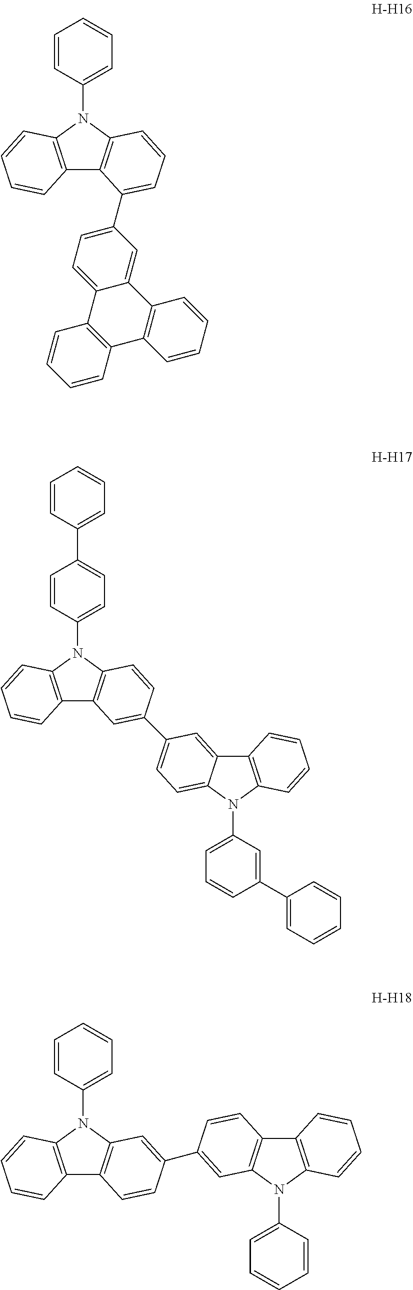 [SMILES: c1ccc(-n2c3ccccc3c3c(-c4ccc5c6ccccc6c6ccccc6c5c4)cccc32)cc1 ; c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-n2c3ccccc3c3ccc(-c4ccc5c6ccccc6n(-c6ccccc6)c5c4)cc32)cc1]

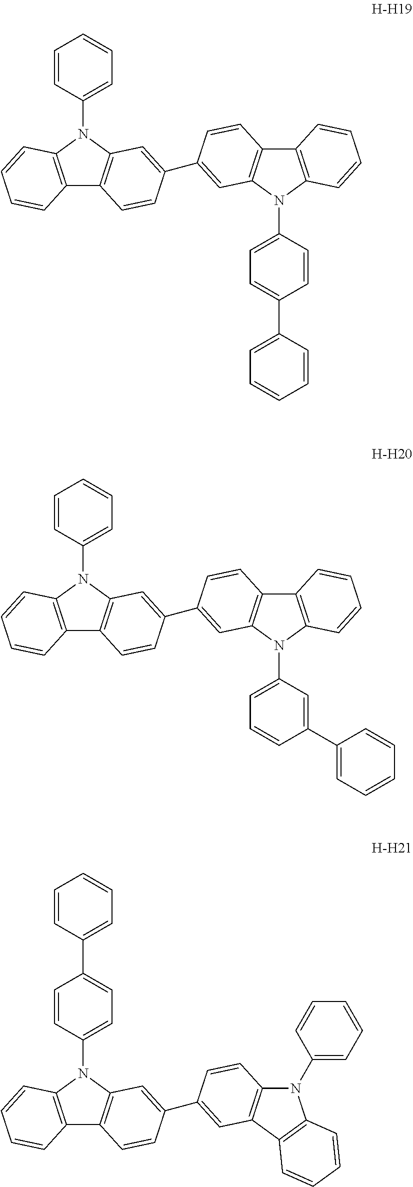 [SMILES: c1ccc(-c2ccc(-n3c4ccccc4c4ccc(-c5ccc6c7ccccc7n(-c7ccccc7)c6c5)cc43)cc2)cc1 ; c1ccc(-c2cccc(-n3c4ccccc4c4ccc(-c5ccc6c7ccccc7n(-c7ccccc7)c6c5)cc43)c2)cc1 ; c1ccc(-c2ccc(-n3c4ccccc4c4ccc(-c5ccc6c(c5)c5ccccc5n6-c5ccccc5)cc43)cc2)cc1]

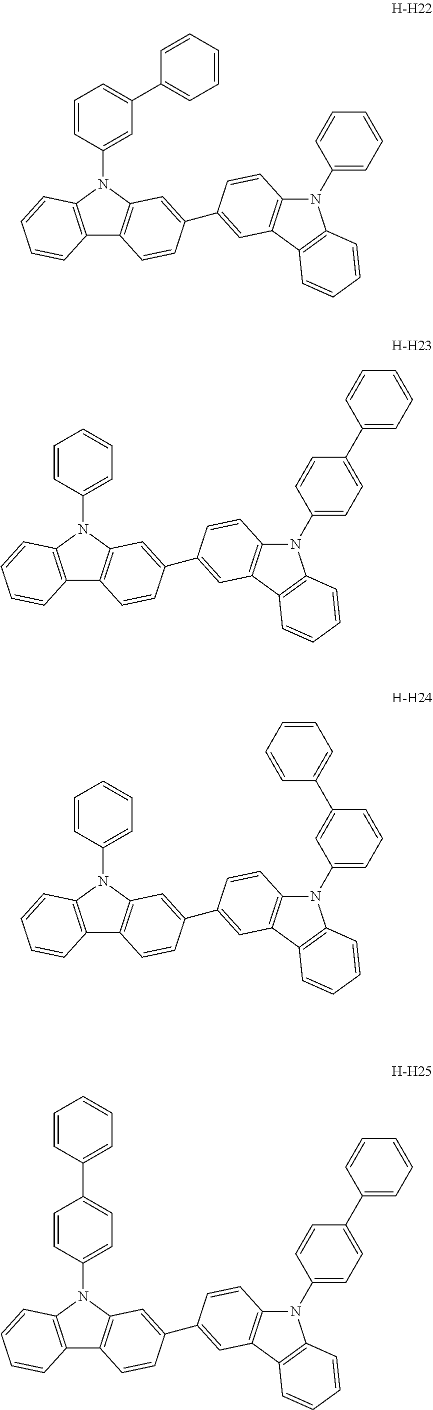 [SMILES: c1ccc(-c2cccc(-n3c4ccccc4c4ccc(-c5ccc6c(c5)c5ccccc5n6-c5ccccc5)cc43)c2)cc1 ; c1ccc(-c2ccc(-n3c4ccccc4c4cc(-c5ccc6c7ccccc7n(-c7ccccc7)c6c5)ccc43)cc2)cc1 ; c1ccc(-c2cccc(-n3c4ccccc4c4cc(-c5ccc6c7ccccc7n(-c7ccccc7)c6c5)ccc43)c2)cc1 ; c1ccc(-c2ccc(-n3c4ccccc4c4cc(-c5ccc6c7ccccc7n(-c7ccc(-c8ccccc8)cc7)c6c5)ccc43)cc2)cc1]

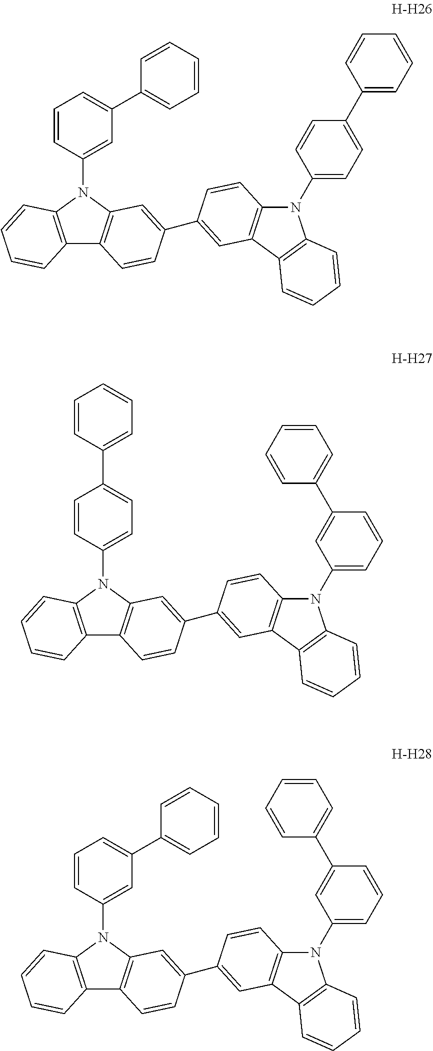 [SMILES: c1ccc(-c2ccc(-n3c4ccccc4c4cc(-c5ccc6c7ccccc7n(-c7cccc(-c8ccccc8)c7)c6c5)ccc43)cc2)cc1 ; c1ccc(-c2ccc(-n3c4ccccc4c4ccc(-c5ccc6c(c5)c5ccccc5n6-c5cccc(-c6ccccc6)c5)cc43)cc2)cc1 ; c1ccc(-c2cccc(-n3c4ccccc4c4cc(-c5ccc6c7ccccc7n(-c7cccc(-c8ccccc8)c7)c6c5)ccc43)c2)cc1]

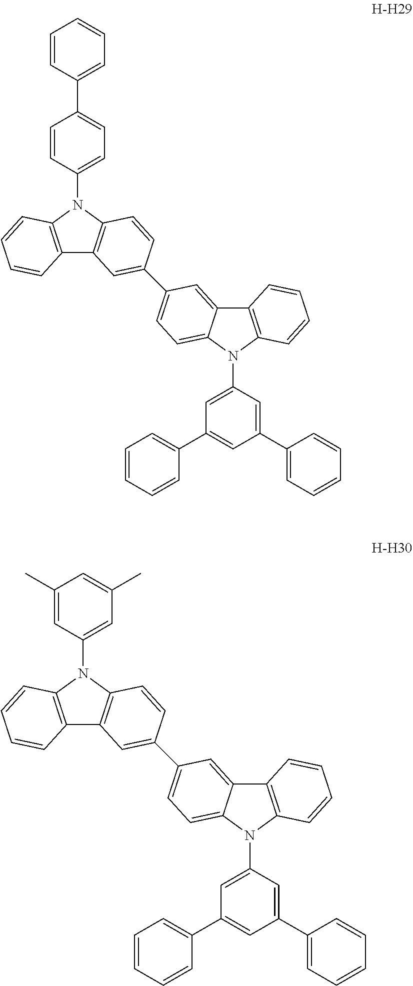 [SMILES: c1ccc(-c2ccc(-n3c4ccccc4c4cc(-c5ccc6c(c5)c5ccccc5n6-c5cc(-c6ccccc6)cc(-c6ccccc6)c5)ccc43)cc2)cc1 ; Cc1cc(C)cc(-n2c3ccccc3c3cc(-c4ccc5c(c4)c4ccccc4n5-c4cc(-c5ccccc5)cc(-c5ccccc5)c4)ccc32)c1]

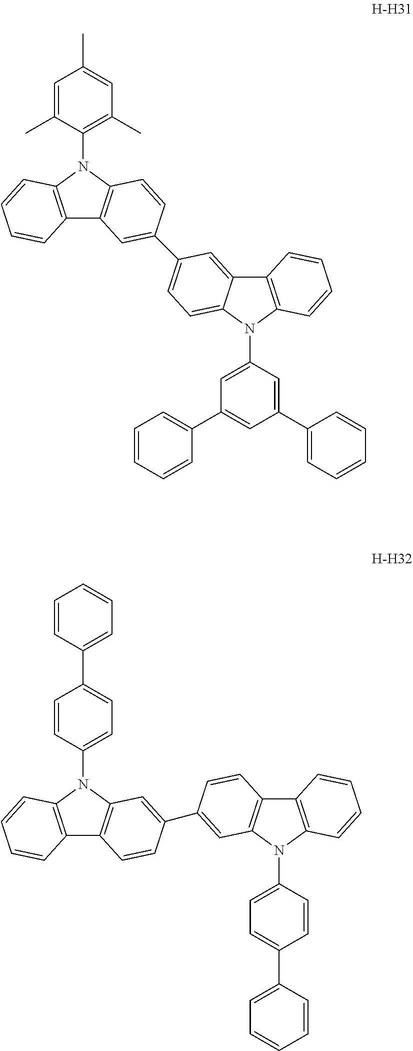 [SMILES: Cc1cc(C)c(-n2c3ccccc3c3cc(-c4ccc5c(c4)c4ccccc4n5-c4cc(-c5ccccc5)cc(-c5ccccc5)c4)ccc32)c(C)c1 ; c1ccc(-c2ccc(-n3c4ccccc4c4ccc(-c5ccc6c7ccccc7n(-c7ccc(-c8ccccc8)cc7)c6c5)cc43)cc2)cc1]

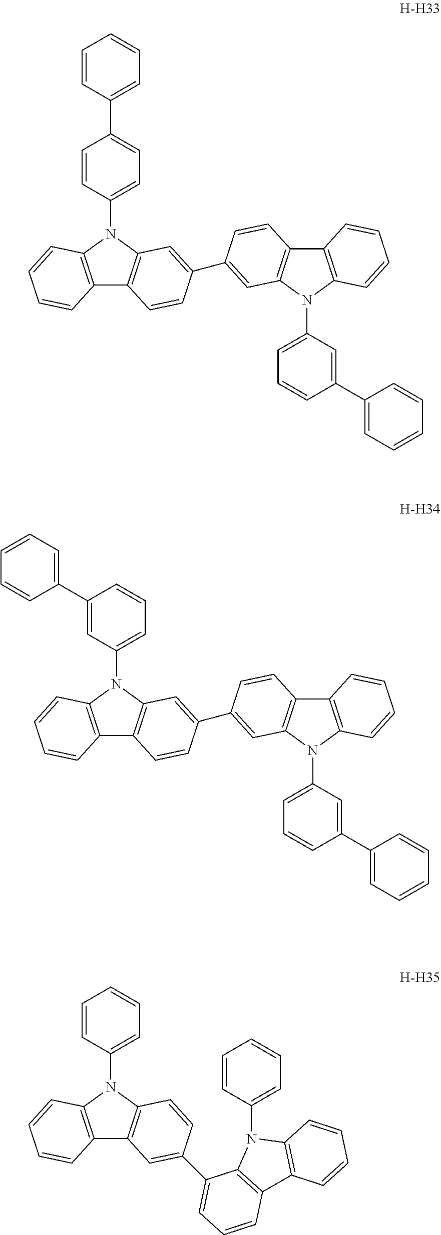 [SMILES: c1ccc(-c2ccc(-n3c4ccccc4c4ccc(-c5ccc6c7ccccc7n(-c7cccc(-c8ccccc8)c7)c6c5)cc43)cc2)cc1 ; c1ccc(-c2cccc(-n3c4ccccc4c4ccc(-c5ccc6c7ccccc7n(-c7cccc(-c8ccccc8)c7)c6c5)cc43)c2)cc1 ; c1ccc(-n2c3ccccc3c3cc(-c4cccc5c6ccccc6n(-c6ccccc6)c45)ccc32)cc1]

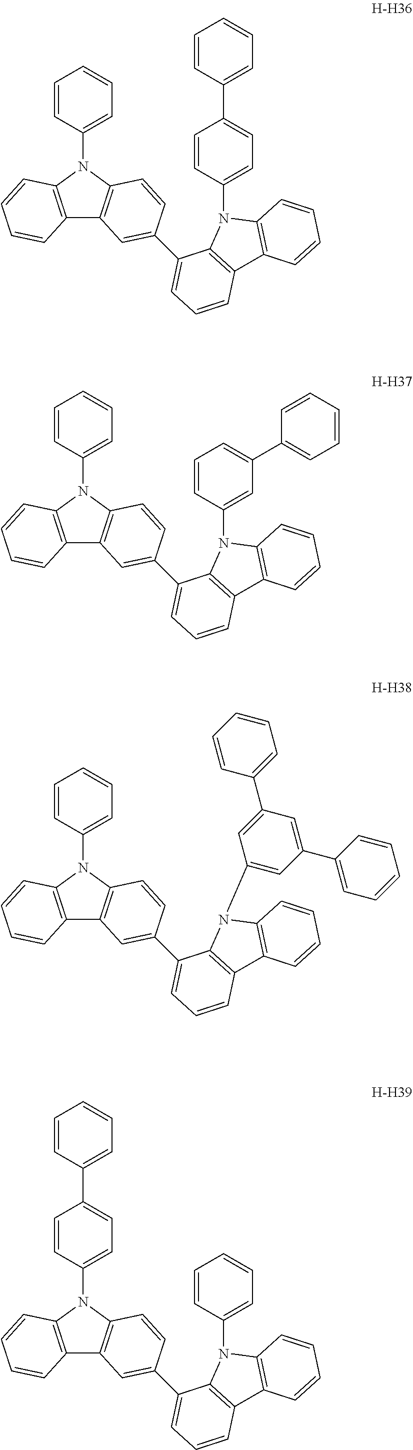 [SMILES: c1ccc(-c2ccc(-n3c4ccccc4c4cccc(-c5ccc6c(c5)c5ccccc5n6-c5ccccc5)c43)cc2)cc1 ; c1ccc(-c2cccc(-n3c4ccccc4c4cccc(-c5ccc6c(c5)c5ccccc5n6-c5ccccc5)c43)c2)cc1 ; c1ccc(-c2cc(-c3ccccc3)cc(-n3c4ccccc4c4cccc(-c5ccc6c(c5)c5ccccc5n6-c5ccccc5)c43)c2)cc1 ; c1ccc(-c2ccc(-n3c4ccccc4c4cc(-c5cccc6c7ccccc7n(-c7ccccc7)c56)ccc43)cc2)cc1]

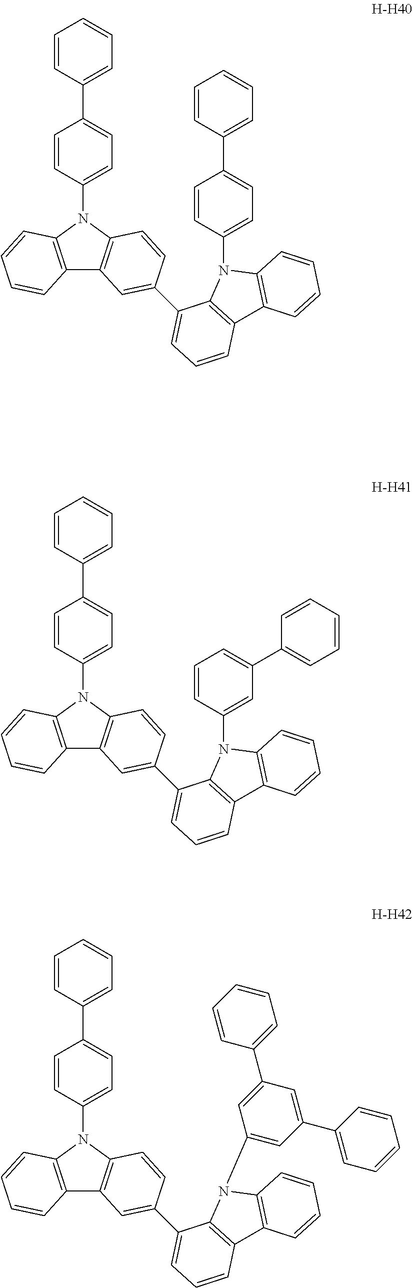 [SMILES: c1ccc(-c2ccc(-n3c4ccccc4c4cc(-c5cccc6c7ccccc7n(-c7ccc(-c8ccccc8)cc7)c56)ccc43)cc2)cc1 ; c1ccc(-c2ccc(-n3c4ccccc4c4cc(-c5cccc6c7ccccc7n(-c7cccc(-c8ccccc8)c7)c56)ccc43)cc2)cc1 ; c1ccc(-c2ccc(-n3c4ccccc4c4cc(-c5cccc6c7ccccc7n(-c7cc(-c8ccccc8)cc(-c8ccccc8)c7)c56)ccc43)cc2)cc1]

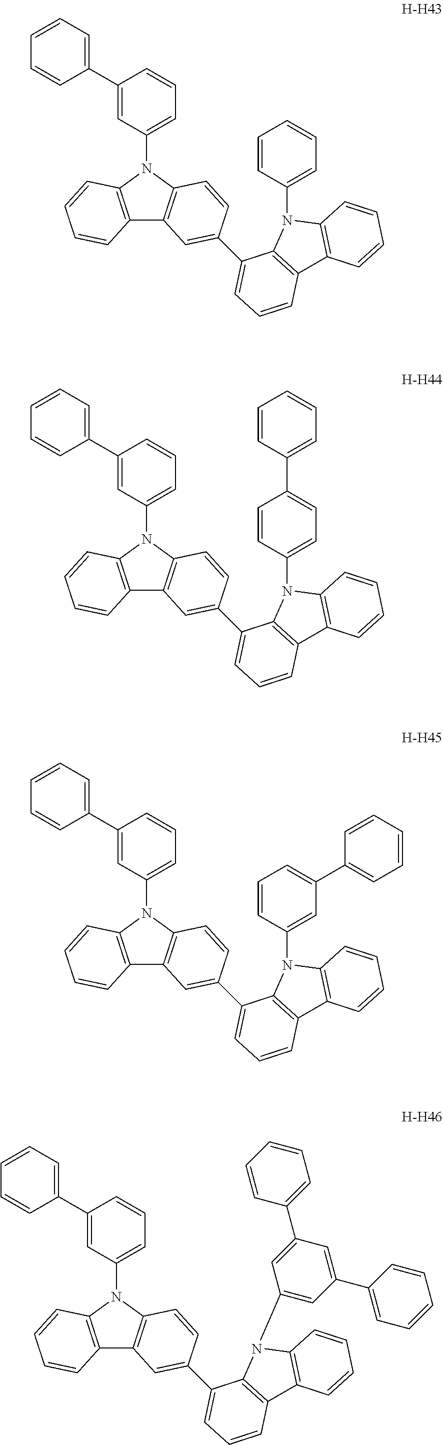 [SMILES: c1ccc(-c2cccc(-n3c4ccccc4c4cc(-c5cccc6c7ccccc7n(-c7ccccc7)c56)ccc43)c2)cc1 ; c1ccc(-c2ccc(-n3c4ccccc4c4cccc(-c5ccc6c(c5)c5ccccc5n6-c5cccc(-c6ccccc6)c5)c43)cc2)cc1 ; c1ccc(-c2cccc(-n3c4ccccc4c4cc(-c5cccc6c7ccccc7n(-c7cccc(-c8ccccc8)c7)c56)ccc43)c2)cc1 ; c1ccc(-c2cccc(-n3c4ccccc4c4cc(-c5cccc6c7ccccc7n(-c7cc(-c8ccccc8)cc(-c8ccccc8)c7)c56)ccc43)c2)cc1]

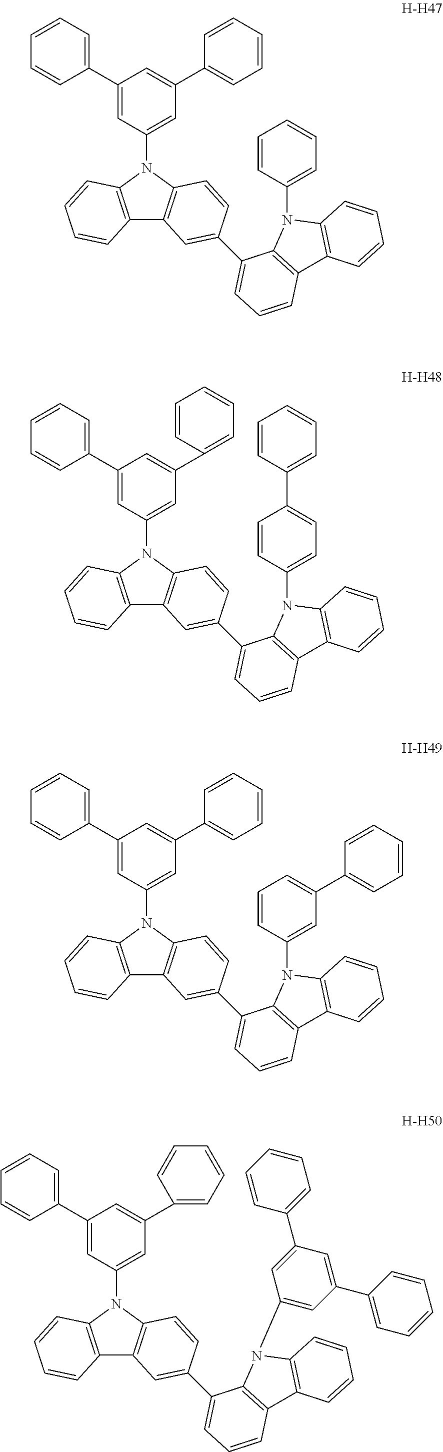 [SMILES: c1ccc(-c2cc(-c3ccccc3)cc(-n3c4ccccc4c4cc(-c5cccc6c7ccccc7n(-c7ccccc7)c56)ccc43)c2)cc1 ; c1ccc(-c2ccc(-n3c4ccccc4c4cccc(-c5ccc6c(c5)c5ccccc5n6-c5cc(-c6ccccc6)cc(-c6ccccc6)c5)c43)cc2)cc1 ; c1ccc(-c2cc(-c3ccccc3)cc(-n3c4ccccc4c4cc(-c5cccc6c7ccccc7n(-c7cccc(-c8ccccc8)c7)c56)ccc43)c2)cc1 ; c1ccc(-c2cc(-c3ccccc3)cc(-n3c4ccccc4c4cc(-c5cccc6c7ccccc7n(-c7cc(-c8ccccc8)cc(-c8ccccc8)c7)c56)ccc43)c2)cc1]

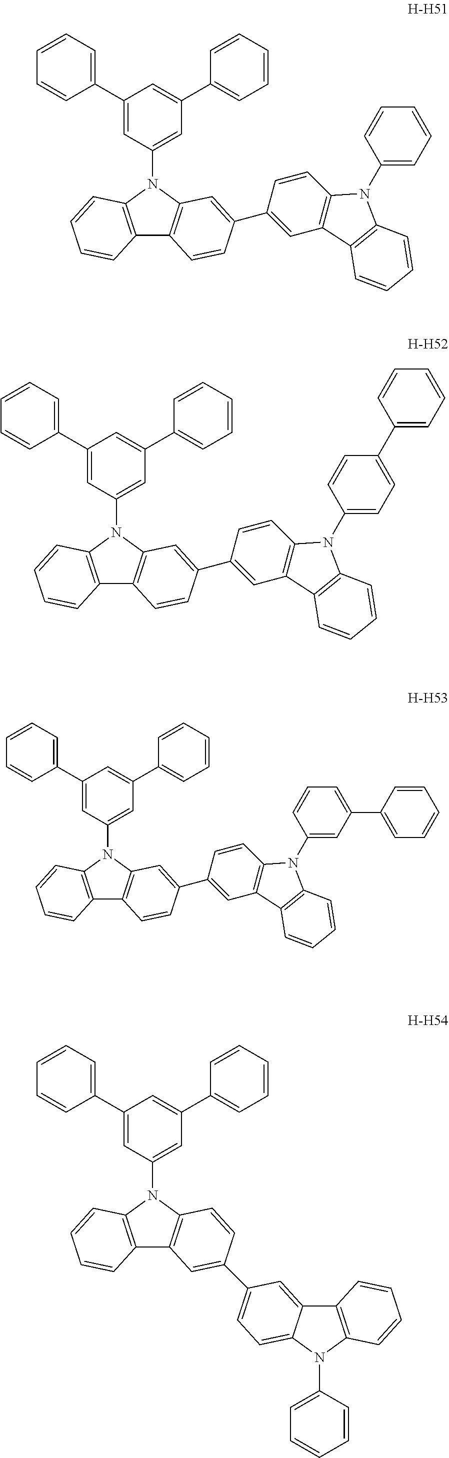 [SMILES: c1ccc(-c2cc(-c3ccccc3)cc(-n3c4ccccc4c4ccc(-c5ccc6c(c5)c5ccccc5n6-c5ccccc5)cc43)c2)cc1 ; c1ccc(-c2ccc(-n3c4ccccc4c4cc(-c5ccc6c7ccccc7n(-c7cc(-c8ccccc8)cc(-c8ccccc8)c7)c6c5)ccc43)cc2)cc1 ; c1ccc(-c2cccc(-n3c4ccccc4c4cc(-c5ccc6c7ccccc7n(-c7cc(-c8ccccc8)cc(-c8ccccc8)c7)c6c5)ccc43)c2)cc1 ; c1ccc(-c2cc(-c3ccccc3)cc(-n3c4ccccc4c4cc(-c5ccc6c(c5)c5ccccc5n6-c5ccccc5)ccc43)c2)cc1]

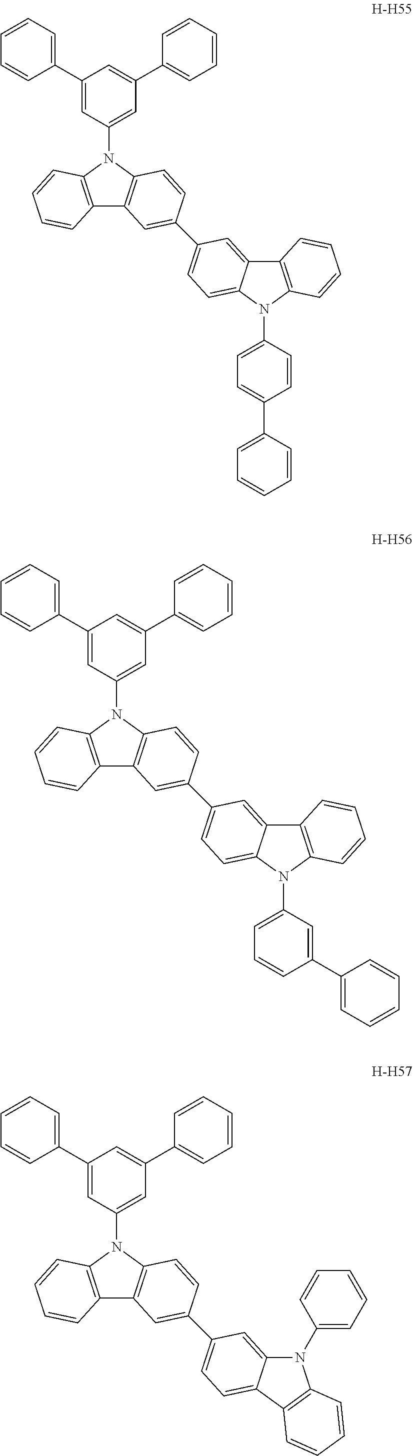 [SMILES: c1ccc(-c2ccc(-n3c4ccccc4c4cc(-c5ccc6c(c5)c5ccccc5n6-c5cc(-c6ccccc6)cc(-c6ccccc6)c5)ccc43)cc2)cc1 ; c1ccc(-c2cccc(-n3c4ccccc4c4cc(-c5ccc6c(c5)c5ccccc5n6-c5cc(-c6ccccc6)cc(-c6ccccc6)c5)ccc43)c2)cc1 ; c1ccc(-c2cc(-c3ccccc3)cc(-n3c4ccccc4c4cc(-c5ccc6c7ccccc7n(-c7ccccc7)c6c5)ccc43)c2)cc1]

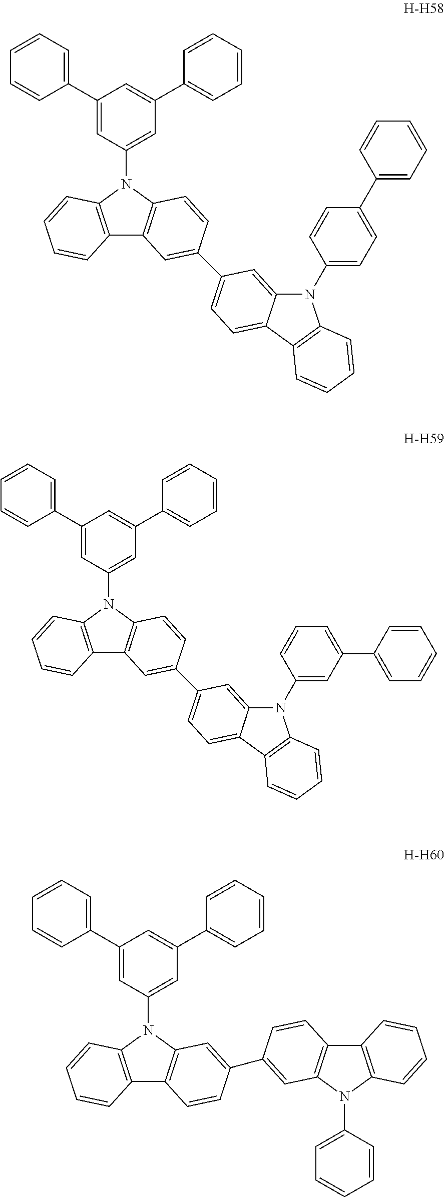 [SMILES: c1ccc(-c2ccc(-n3c4ccccc4c4ccc(-c5ccc6c(c5)c5ccccc5n6-c5cc(-c6ccccc6)cc(-c6ccccc6)c5)cc43)cc2)cc1 ; c1ccc(-c2cccc(-n3c4ccccc4c4ccc(-c5ccc6c(c5)c5ccccc5n6-c5cc(-c6ccccc6)cc(-c6ccccc6)c5)cc43)c2)cc1 ; c1ccc(-c2cc(-c3ccccc3)cc(-n3c4ccccc4c4ccc(-c5ccc6c7ccccc7n(-c7ccccc7)c6c5)cc43)c2)cc1]

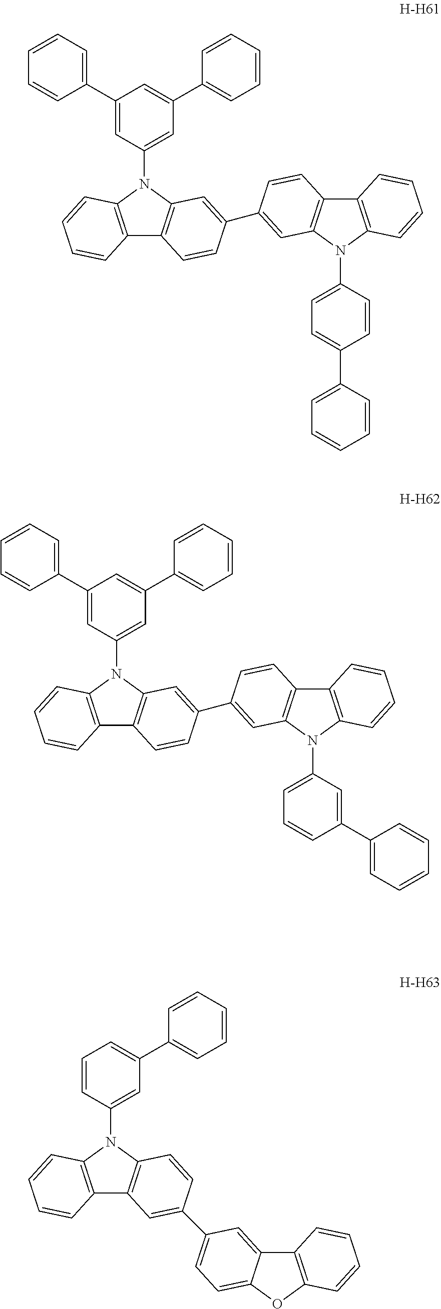 [SMILES: c1ccc(-c2ccc(-n3c4ccccc4c4ccc(-c5ccc6c7ccccc7n(-c7cc(-c8ccccc8)cc(-c8ccccc8)c7)c6c5)cc43)cc2)cc1 ; c1ccc(-c2cccc(-n3c4ccccc4c4ccc(-c5ccc6c7ccccc7n(-c7cc(-c8ccccc8)cc(-c8ccccc8)c7)c6c5)cc43)c2)cc1 ; c1ccc(-c2cccc(-n3c4ccccc4c4cc(-c5ccc6oc7ccccc7c6c5)ccc43)c2)cc1]

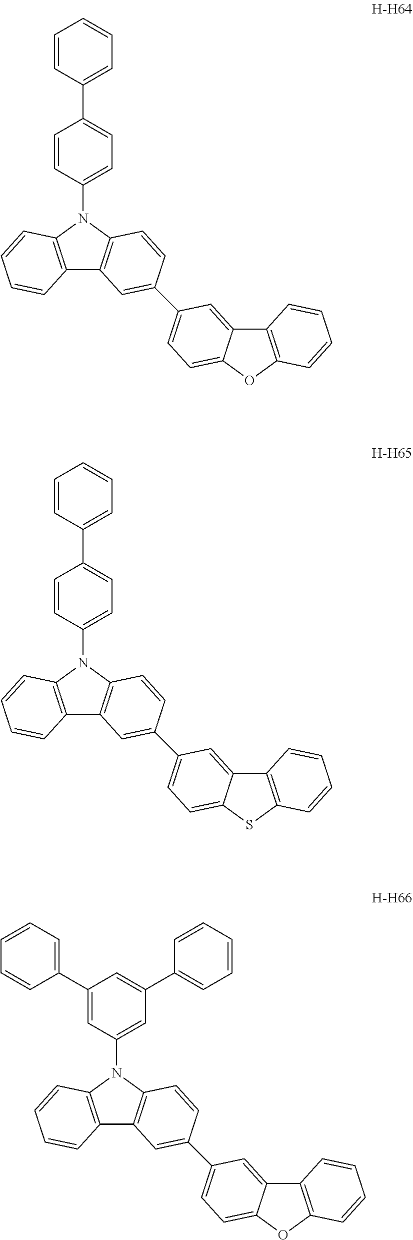 [SMILES: c1ccc(-c2ccc(-n3c4ccccc4c4cc(-c5ccc6oc7ccccc7c6c5)ccc43)cc2)cc1 ; c1ccc(-c2ccc(-n3c4ccccc4c4cc(-c5ccc6sc7ccccc7c6c5)ccc43)cc2)cc1 ; c1ccc(-c2cc(-c3ccccc3)cc(-n3c4ccccc4c4cc(-c5ccc6oc7ccccc7c6c5)ccc43)c2)cc1]

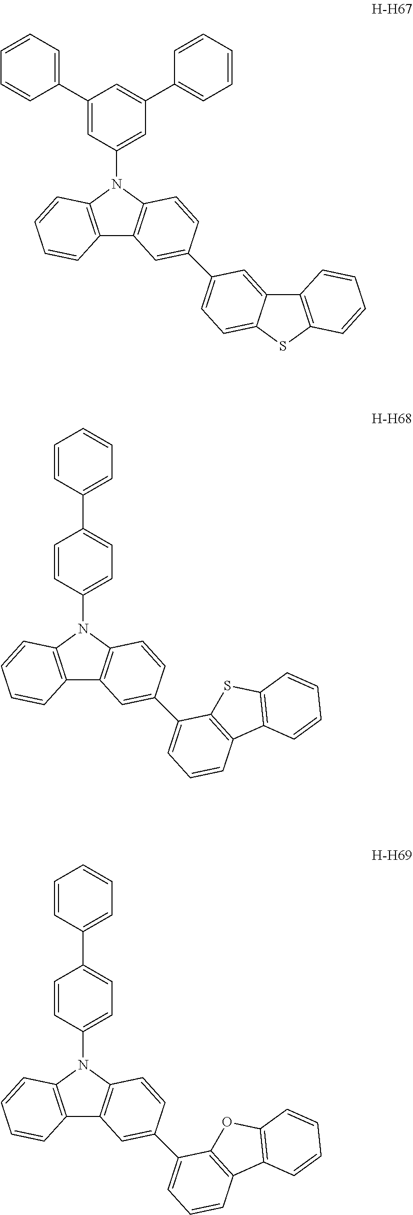 [SMILES: c1ccc(-c2cc(-c3ccccc3)cc(-n3c4ccccc4c4cc(-c5ccc6sc7ccccc7c6c5)ccc43)c2)cc1 ; c1ccc(-c2ccc(-n3c4ccccc4c4cc(-c5cccc6c5sc5ccccc56)ccc43)cc2)cc1 ; c1ccc(-c2ccc(-n3c4ccccc4c4cc(-c5cccc6c5oc5ccccc56)ccc43)cc2)cc1]

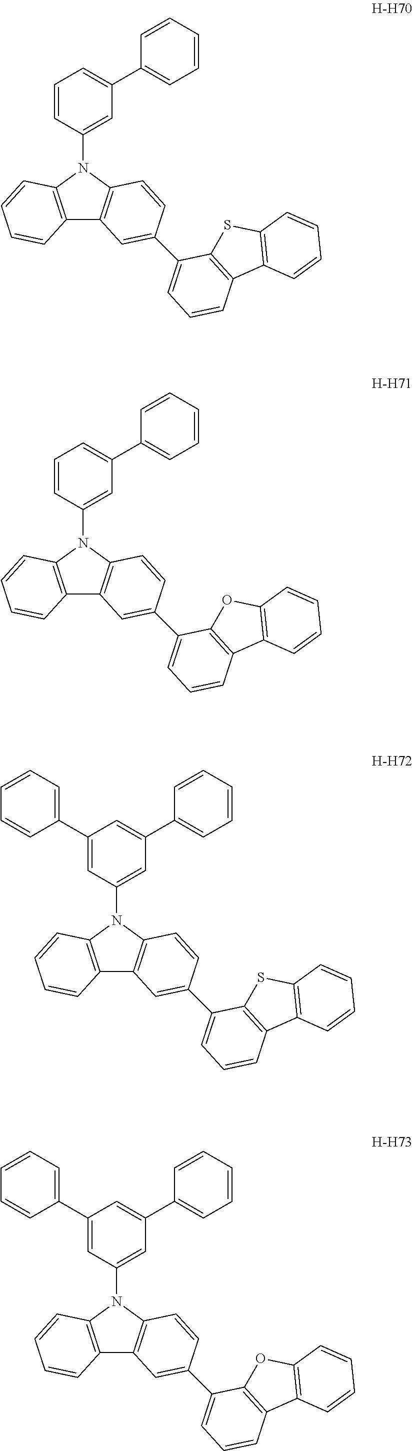 [SMILES: c1ccc(-c2cccc(-n3c4ccccc4c4cc(-c5cccc6c5sc5ccccc56)ccc43)c2)cc1 ; c1ccc(-c2cccc(-n3c4ccccc4c4cc(-c5cccc6c5oc5ccccc56)ccc43)c2)cc1 ; c1ccc(-c2cc(-c3ccccc3)cc(-n3c4ccccc4c4cc(-c5cccc6c5sc5ccccc56)ccc43)c2)cc1 ; c1ccc(-c2cc(-c3ccccc3)cc(-n3c4ccccc4c4cc(-c5cccc6c5oc5ccccc56)ccc43)c2)cc1]

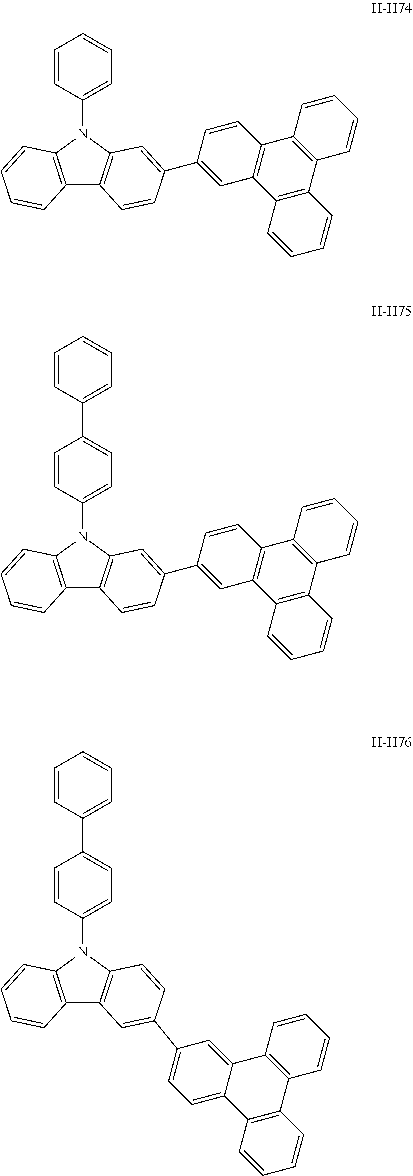 [SMILES: c1ccc(-n2c3ccccc3c3ccc(-c4ccc5c6ccccc6c6ccccc6c5c4)cc32)cc1 ; c1ccc(-c2ccc(-n3c4ccccc4c4ccc(-c5ccc6c7ccccc7c7ccccc7c6c5)cc43)cc2)cc1 ; c1ccc(-c2ccc(-n3c4ccccc4c4cc(-c5ccc6c7ccccc7c7ccccc7c6c5)ccc43)cc2)cc1]

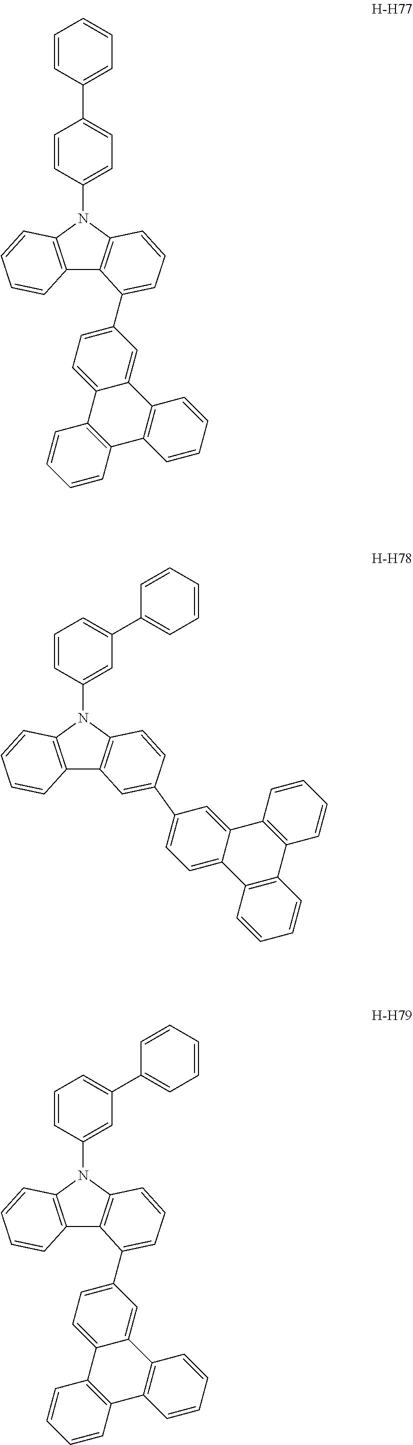 [SMILES: c1ccc(-c2ccc(-n3c4ccccc4c4c(-c5ccc6c7ccccc7c7ccccc7c6c5)cccc43)cc2)cc1 ; c1ccc(-c2cccc(-n3c4ccccc4c4cc(-c5ccc6c7ccccc7c7ccccc7c6c5)ccc43)c2)cc1 ; c1ccc(-c2cccc(-n3c4ccccc4c4c(-c5ccc6c7ccccc7c7ccccc7c6c5)cccc43)c2)cc1]

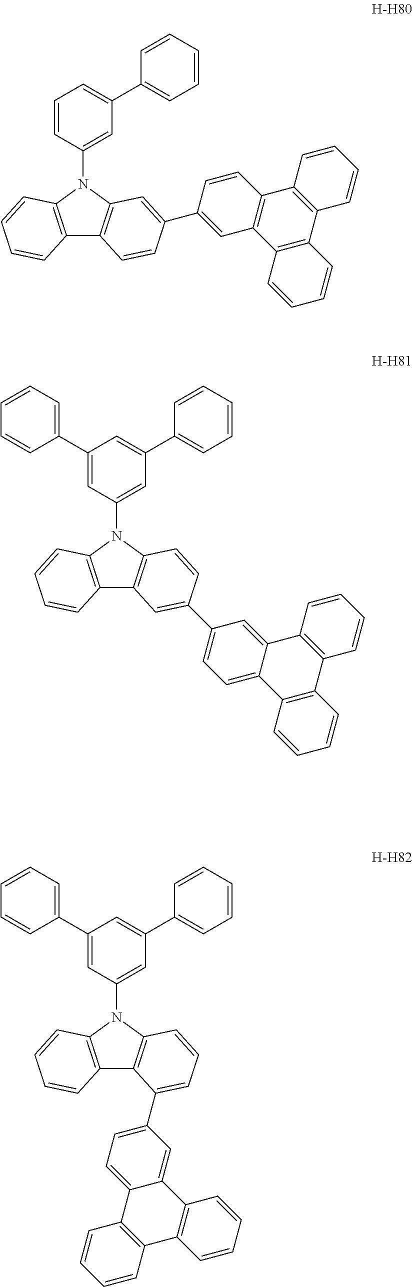 [SMILES: c1ccc(-c2cccc(-n3c4ccccc4c4ccc(-c5ccc6c7ccccc7c7ccccc7c6c5)cc43)c2)cc1 ; c1ccc(-c2cc(-c3ccccc3)cc(-n3c4ccccc4c4cc(-c5ccc6c7ccccc7c7ccccc7c6c5)ccc43)c2)cc1 ; c1ccc(-c2cc(-c3ccccc3)cc(-n3c4ccccc4c4c(-c5ccc6c7ccccc7c7ccccc7c6c5)cccc43)c2)cc1]

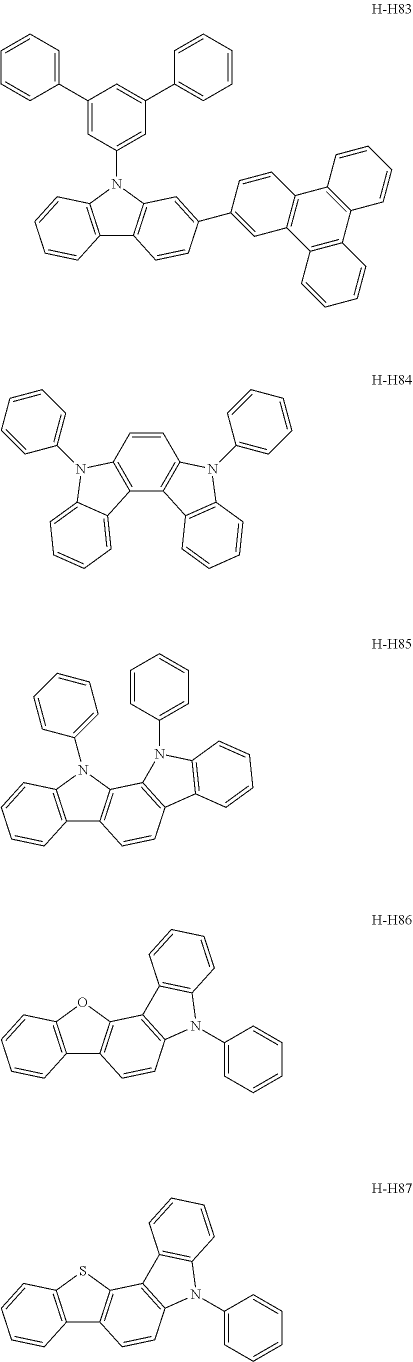 [SMILES: c1ccc(-c2cc(-c3ccccc3)cc(-n3c4ccccc4c4ccc(-c5ccc6c7ccccc7c7ccccc7c6c5)cc43)c2)cc1 ; c1ccc(-n2c3ccccc3c3c4c5ccccc5n(-c5ccccc5)c4ccc32)cc1 ; c1ccc(-n2c3ccccc3c3ccc4c5ccccc5n(-c5ccccc5)c4c32)cc1 ; c1ccc(-n2c3ccccc3c3c4oc5ccccc5c4ccc32)cc1 ; c1ccc(-n2c3ccccc3c3c4sc5ccccc5c4ccc32)cc1]

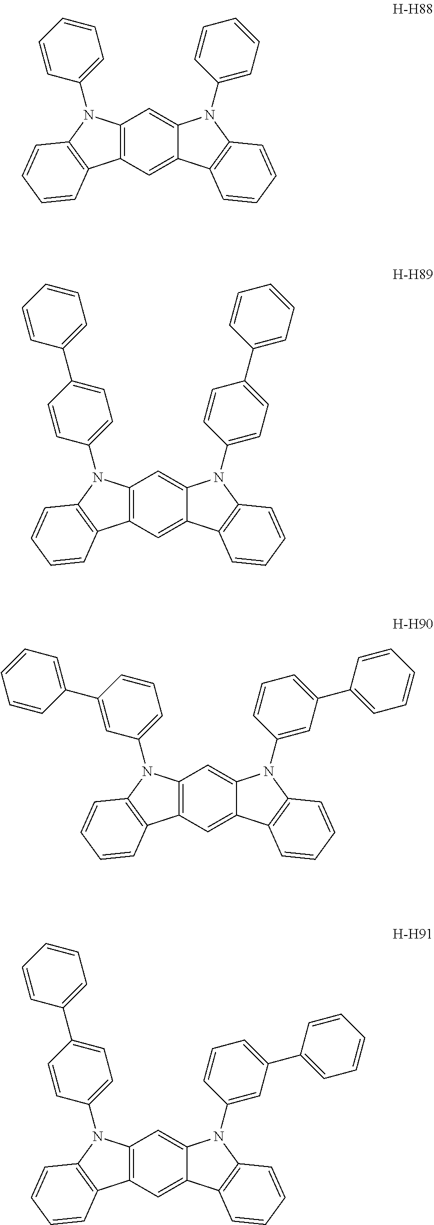 [SMILES: c1ccc(-n2c3ccccc3c3cc4c5ccccc5n(-c5ccccc5)c4cc32)cc1 ; c1ccc(-c2ccc(-n3c4ccccc4c4cc5c6ccccc6n(-c6ccc(-c7ccccc7)cc6)c5cc43)cc2)cc1 ; c1ccc(-c2cccc(-n3c4ccccc4c4cc5c6ccccc6n(-c6cccc(-c7ccccc7)c6)c5cc43)c2)cc1 ; c1ccc(-c2ccc(-n3c4ccccc4c4cc5c6ccccc6n(-c6cccc(-c7ccccc7)c6)c5cc43)cc2)cc1]

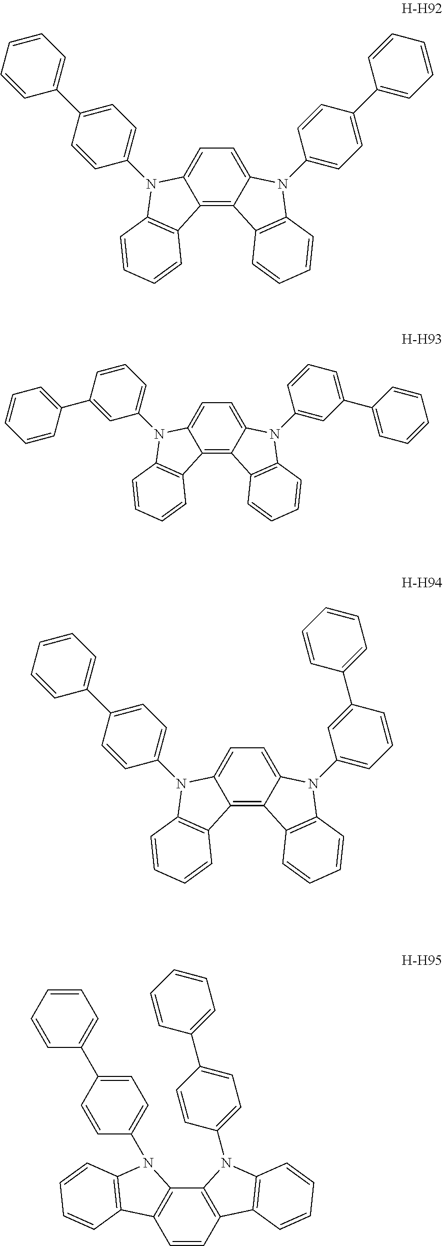 [SMILES: c1ccc(-c2ccc(-n3c4ccccc4c4c5c6ccccc6n(-c6ccc(-c7ccccc7)cc6)c5ccc43)cc2)cc1 ; c1ccc(-c2cccc(-n3c4ccccc4c4c5c6ccccc6n(-c6cccc(-c7ccccc7)c6)c5ccc43)c2)cc1 ; c1ccc(-c2ccc(-n3c4ccccc4c4c5c6ccccc6n(-c6cccc(-c7ccccc7)c6)c5ccc43)cc2)cc1 ; c1ccc(-c2ccc(-n3c4ccccc4c4ccc5c6ccccc6n(-c6ccc(-c7ccccc7)cc6)c5c43)cc2)cc1]

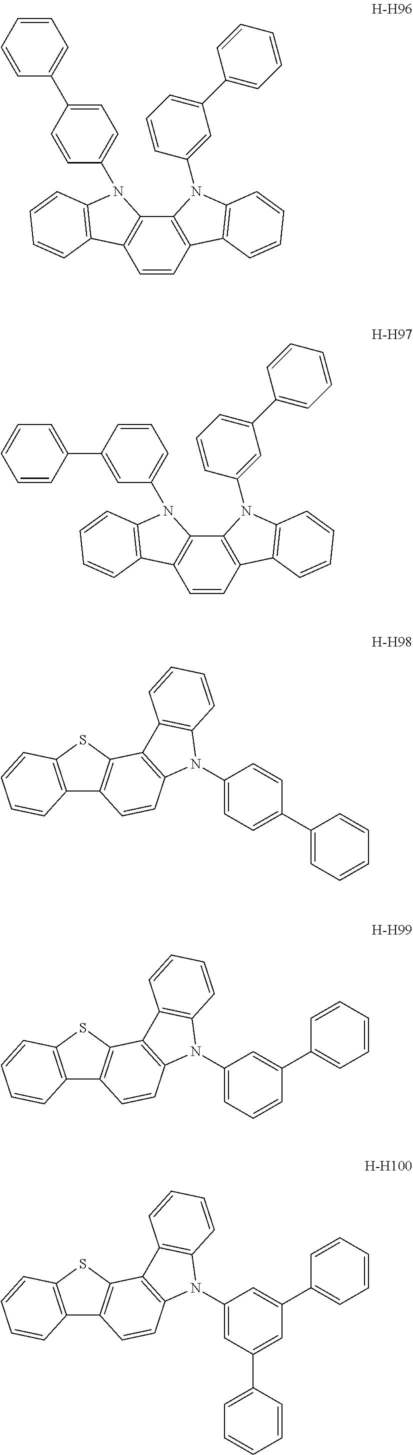 [SMILES: c1ccc(-c2ccc(-n3c4ccccc4c4ccc5c6ccccc6n(-c6cccc(-c7ccccc7)c6)c5c43)cc2)cc1 ; c1ccc(-c2cccc(-n3c4ccccc4c4ccc5c6ccccc6n(-c6cccc(-c7ccccc7)c6)c5c43)c2)cc1 ; c1ccc(-c2ccc(-n3c4ccccc4c4c5sc6ccccc6c5ccc43)cc2)cc1 ; c1ccc(-c2cccc(-n3c4ccccc4c4c5sc6ccccc6c5ccc43)c2)cc1 ; c1ccc(-c2cc(-c3ccccc3)cc(-n3c4ccccc4c4c5sc6ccccc6c5ccc43)c2)cc1]

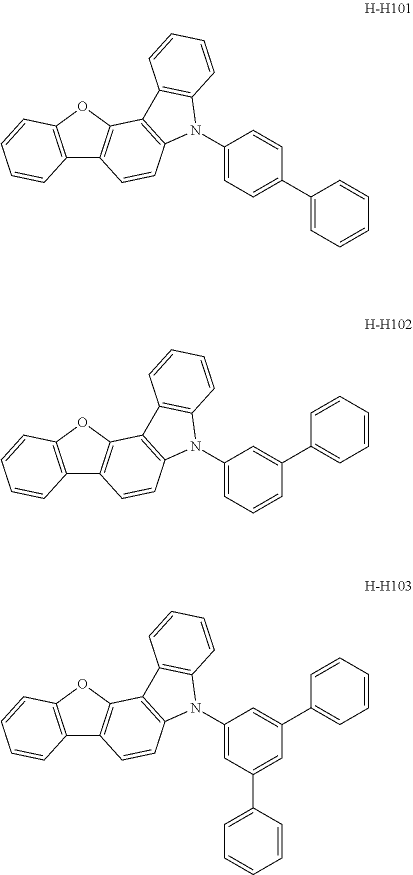 [SMILES: c1ccc(-c2ccc(-n3c4ccccc4c4c5oc6ccccc6c5ccc43)cc2)cc1 ; c1ccc(-c2cccc(-n3c4ccccc4c4c5oc6ccccc6c5ccc43)c2)cc1 ; c1ccc(-c2cc(-c3ccccc3)cc(-n3c4ccccc4c4c5oc6ccccc6c5ccc43)c2)cc1]

In some embodiments, the bipolar host may be Group HEH1, but embodiments are not limited thereto:

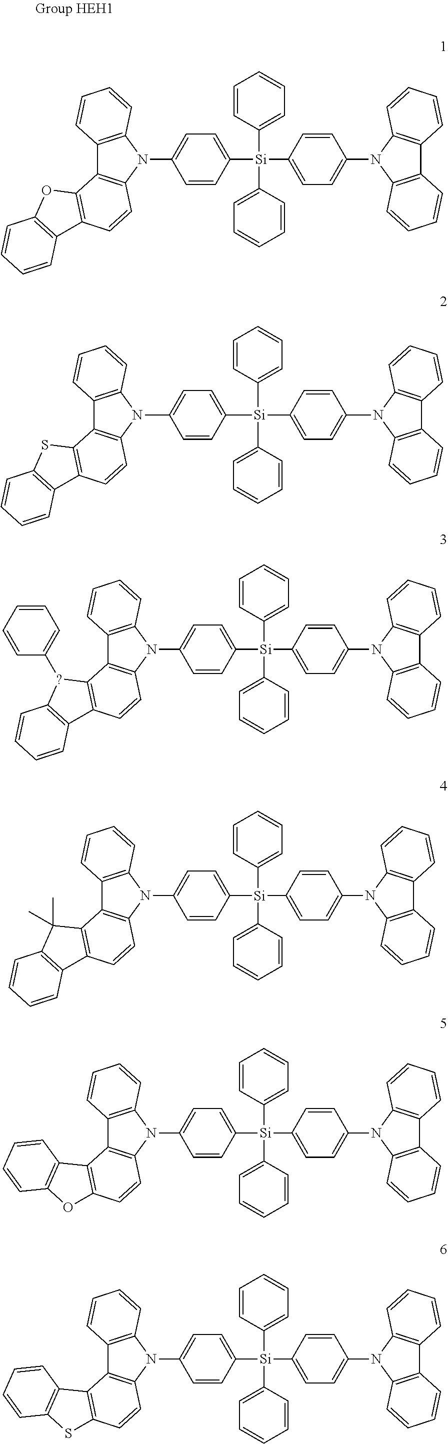

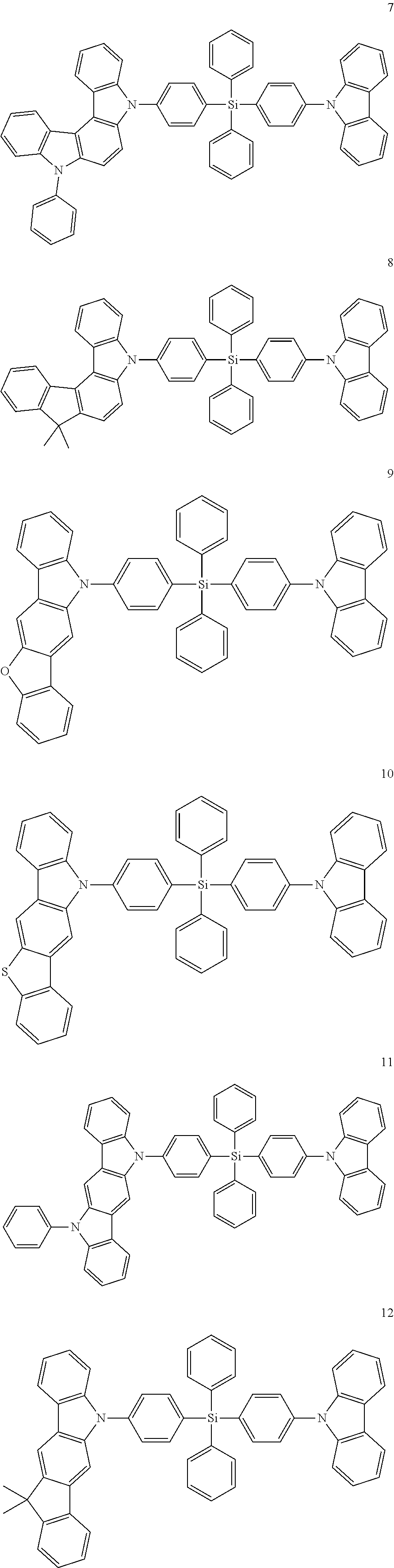

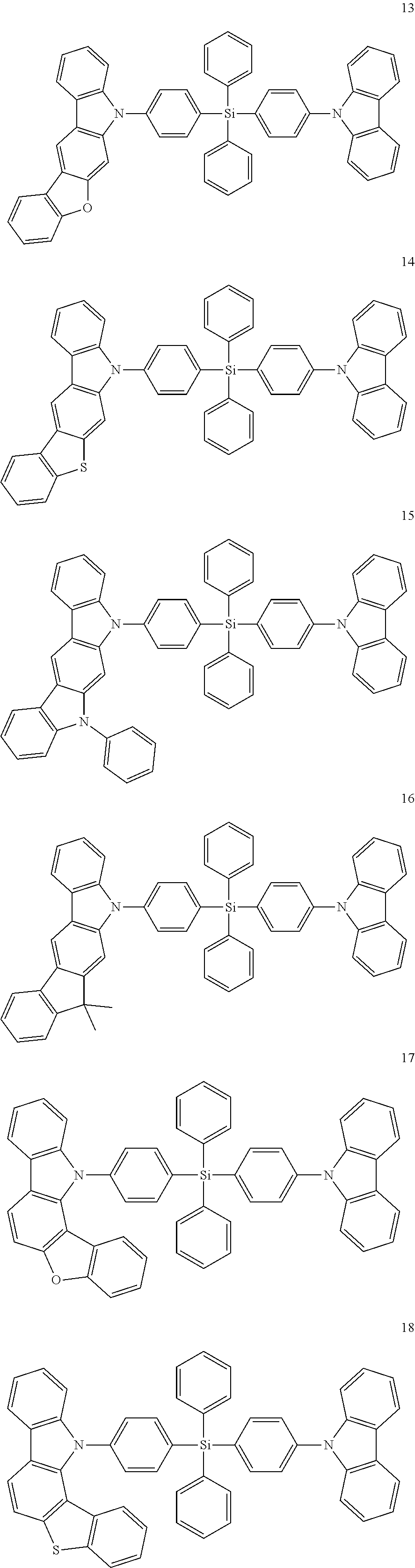

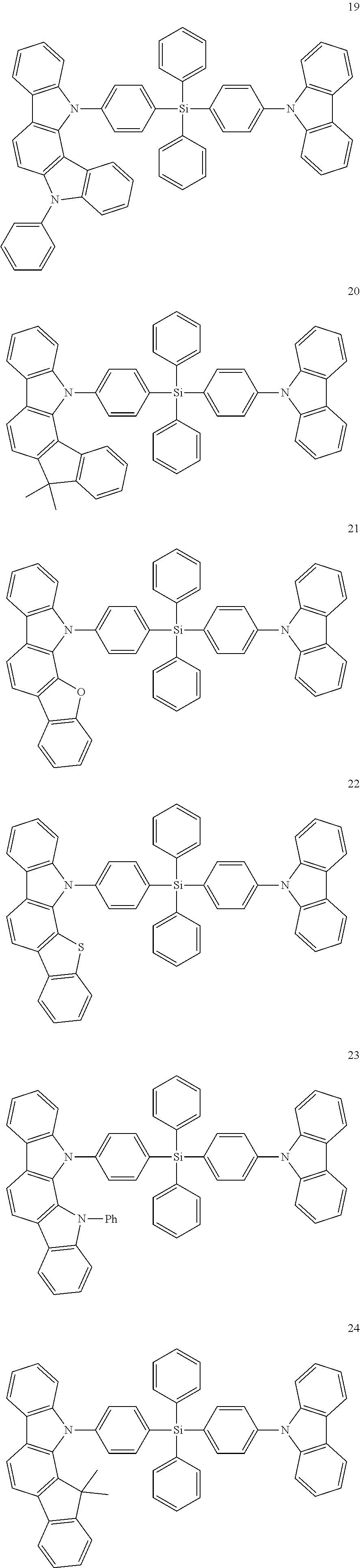

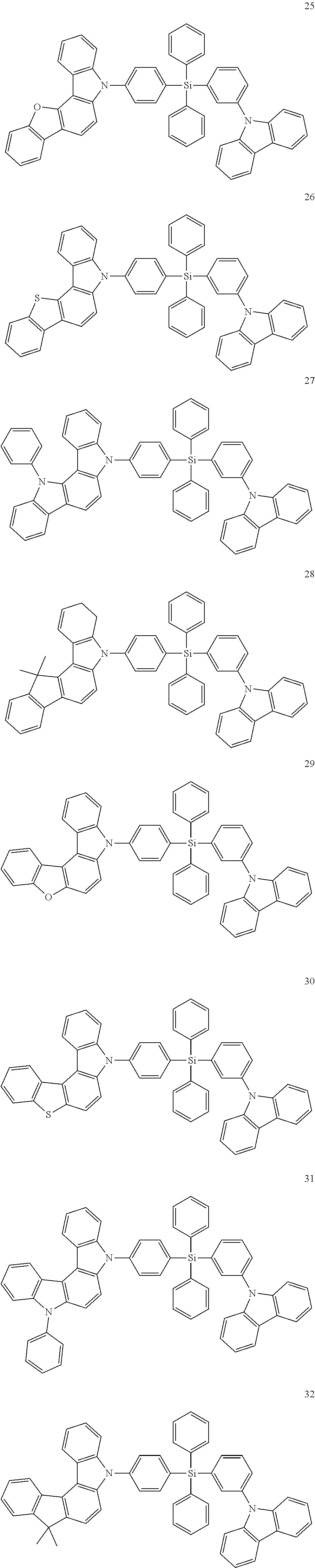

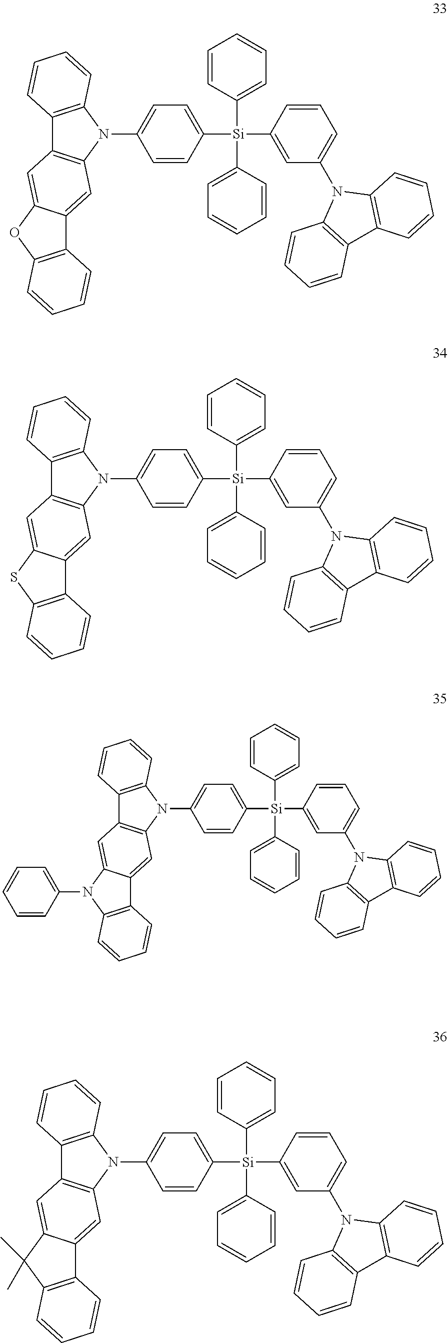 [SMILES: c1ccc([Si](c2ccccc2)(c2ccc(-n3c4ccccc4c4cc5oc6ccccc6c5cc43)cc2)c2cccc(-n3c4ccccc4c4ccccc43)c2)cc1 ; c1ccc([Si](c2ccccc2)(c2ccc(-n3c4ccccc4c4cc5sc6ccccc6c5cc43)cc2)c2cccc(-n3c4ccccc4c4ccccc43)c2)cc1 ; c1ccc(-n2c3ccccc3c3cc4c(cc32)c2ccccc2n4-c2ccc([Si](c3ccccc3)(c3ccccc3)c3cccc(-n4c5ccccc5c5ccccc54)c3)cc2)cc1 ; CC1(C)c2ccccc2-c2cc3c(cc21)c1ccccc1n3-c1ccc([Si](c2ccccc2)(c2ccccc2)c2cccc(-n3c4ccccc4c4ccccc43)c2)cc1]

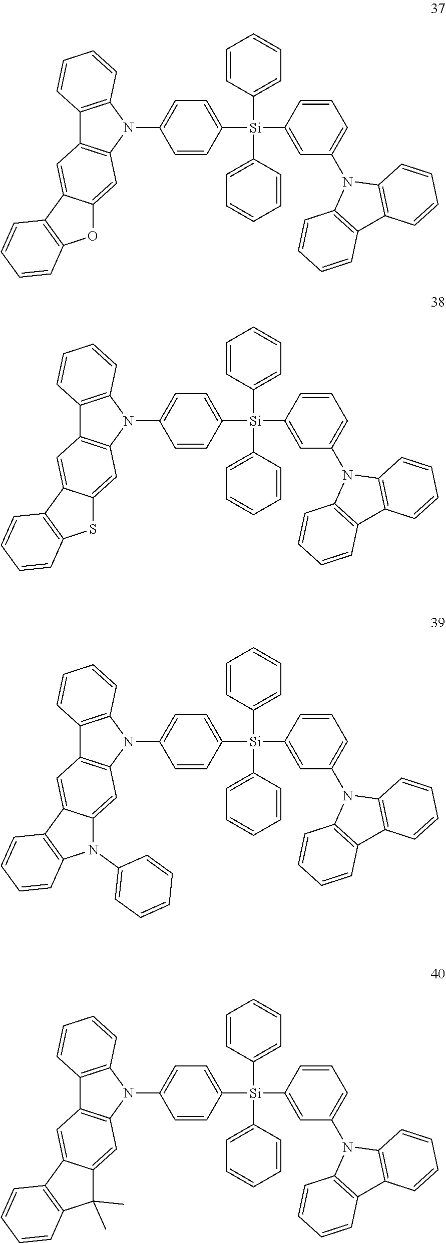 [SMILES: c1ccc([Si](c2ccccc2)(c2ccc(-n3c4ccccc4c4cc5c(cc43)oc3ccccc35)cc2)c2cccc(-n3c4ccccc4c4ccccc43)c2)cc1 ; c1ccc([Si](c2ccccc2)(c2ccc(-n3c4ccccc4c4cc5c(cc43)sc3ccccc35)cc2)c2cccc(-n3c4ccccc4c4ccccc43)c2)cc1 ; c1ccc(-n2c3ccccc3c3cc4c5ccccc5n(-c5ccc([Si](c6ccccc6)(c6ccccc6)c6cccc(-n7c8ccccc8c8ccccc87)c6)cc5)c4cc32)cc1 ; CC1(C)c2ccccc2-c2cc3c4ccccc4n(-c4ccc([Si](c5ccccc5)(c5ccccc5)c5cccc(-n6c7ccccc7c7ccccc76)c5)cc4)c3cc21]

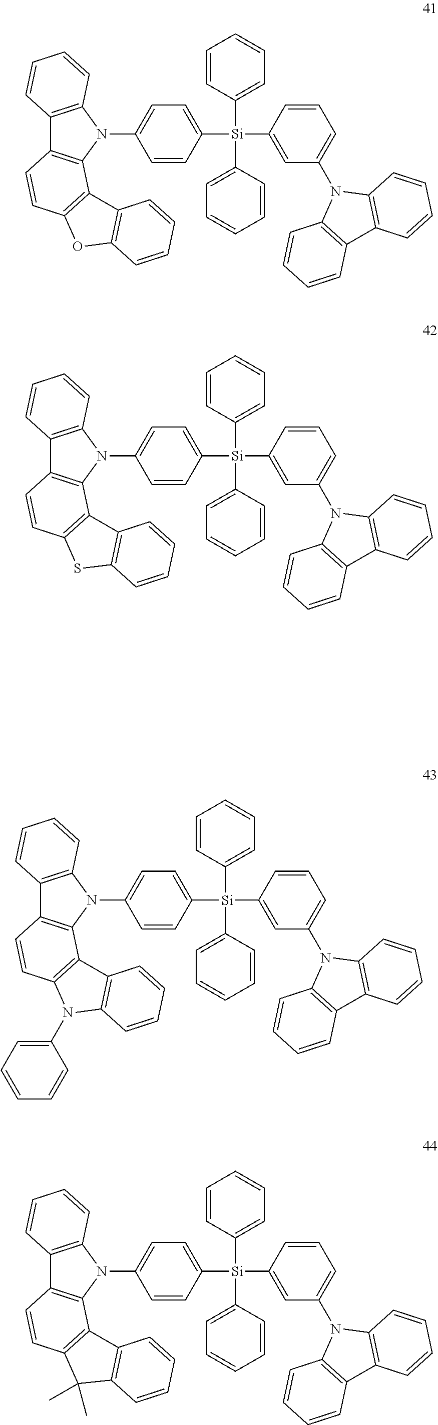 [SMILES: c1ccc([Si](c2ccccc2)(c2ccc(-n3c4ccccc4c4ccc5oc6ccccc6c5c43)cc2)c2cccc(-n3c4ccccc4c4ccccc43)c2)cc1 ; c1ccc([Si](c2ccccc2)(c2ccc(-n3c4ccccc4c4ccc5sc6ccccc6c5c43)cc2)c2cccc(-n3c4ccccc4c4ccccc43)c2)cc1 ; c1ccc(-n2c3ccccc3c3c2ccc2c4ccccc4n(-c4ccc([Si](c5ccccc5)(c5ccccc5)c5cccc(-n6c7ccccc7c7ccccc76)c5)cc4)c23)cc1 ; CC1(C)c2ccccc2-c2c1ccc1c3ccccc3n(-c3ccc([Si](c4ccccc4)(c4ccccc4)c4cccc(-n5c6ccccc6c6ccccc65)c4)cc3)c21]

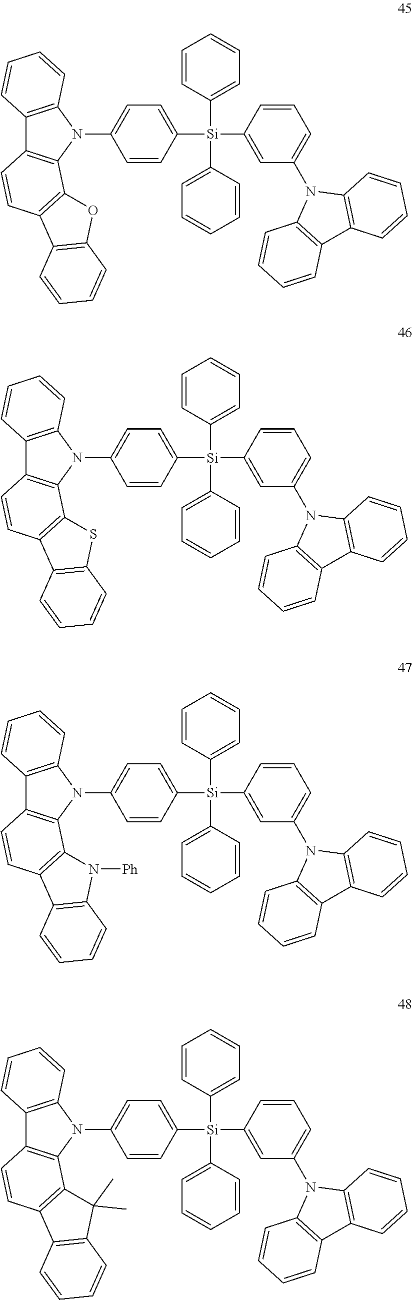 [SMILES: c1ccc([Si](c2ccccc2)(c2ccc(-n3c4ccccc4c4ccc5c6ccccc6oc5c43)cc2)c2cccc(-n3c4ccccc4c4ccccc43)c2)cc1 ; c1ccc([Si](c2ccccc2)(c2ccc(-n3c4ccccc4c4ccc5c6ccccc6sc5c43)cc2)c2cccc(-n3c4ccccc4c4ccccc43)c2)cc1 ; c1ccc(-n2c3ccccc3c3ccc4c5ccccc5n(-c5ccc([Si](c6ccccc6)(c6ccccc6)c6cccc(-n7c8ccccc8c8ccccc87)c6)cc5)c4c32)cc1 ; CC1(C)c2ccccc2-c2ccc3c4ccccc4n(-c4ccc([Si](c5ccccc5)(c5ccccc5)c5cccc(-n6c7ccccc7c7ccccc76)c5)cc4)c3c21]

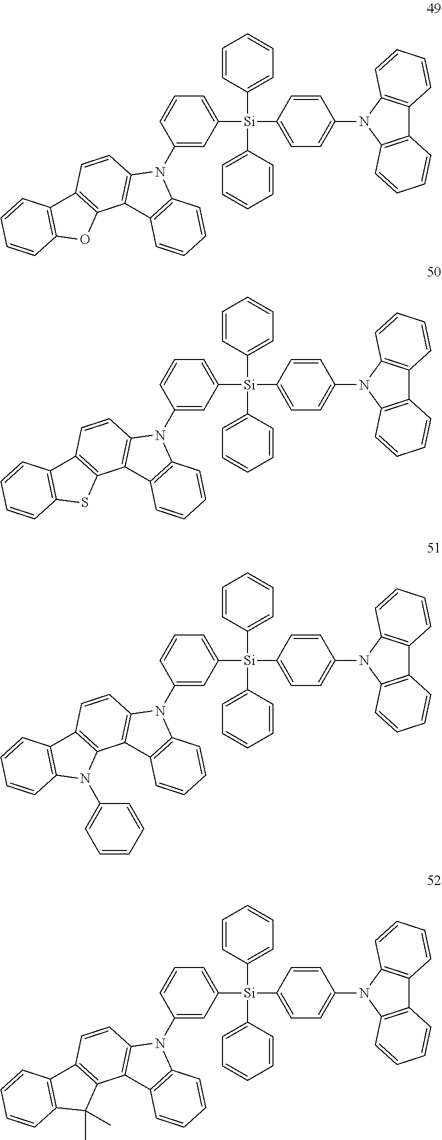 [SMILES: c1ccc([Si](c2ccccc2)(c2ccc(-n3c4ccccc4c4ccccc43)cc2)c2cccc(-n3c4ccccc4c4c5oc6ccccc6c5ccc43)c2)cc1 ; c1ccc([Si](c2ccccc2)(c2ccc(-n3c4ccccc4c4ccccc43)cc2)c2cccc(-n3c4ccccc4c4c5sc6ccccc6c5ccc43)c2)cc1 ; c1ccc(-n2c3ccccc3c3ccc4c(c5ccccc5n4-c4cccc([Si](c5ccccc5)(c5ccccc5)c5ccc(-n6c7ccccc7c7ccccc76)cc5)c4)c32)cc1 ; CC1(C)c2ccccc2-c2ccc3c(c21)c1ccccc1n3-c1cccc([Si](c2ccccc2)(c2ccccc2)c2ccc(-n3c4ccccc4c4ccccc43)cc2)c1]

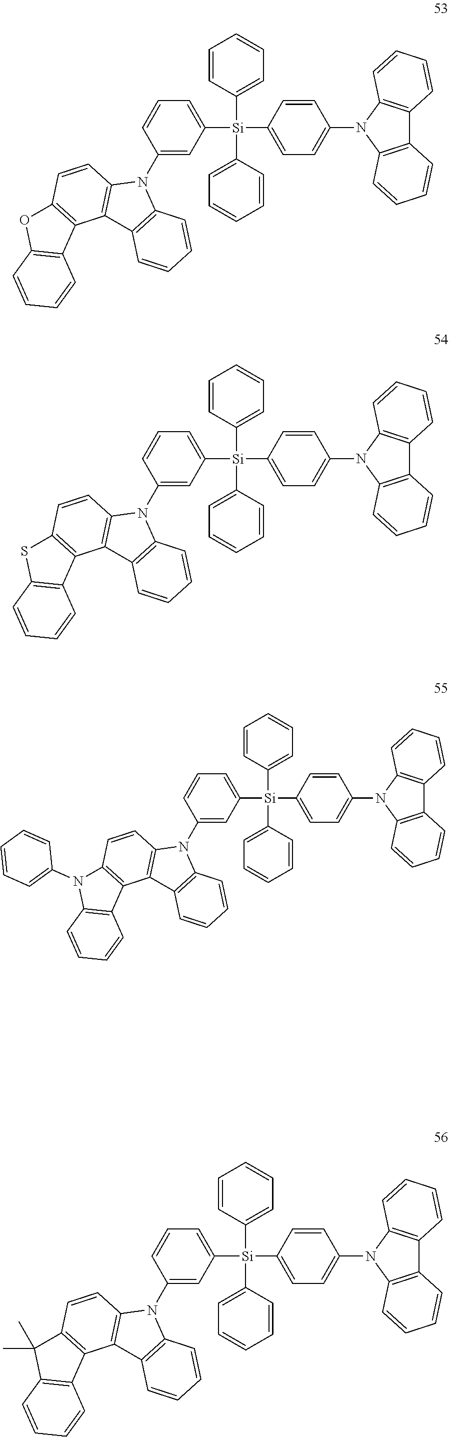 [SMILES: c1ccc([Si](c2ccccc2)(c2ccc(-n3c4ccccc4c4ccccc43)cc2)c2cccc(-n3c4ccccc4c4c5c(ccc43)oc3ccccc35)c2)cc1 ; c1ccc([Si](c2ccccc2)(c2ccc(-n3c4ccccc4c4ccccc43)cc2)c2cccc(-n3c4ccccc4c4c5c(ccc43)sc3ccccc35)c2)cc1 ; c1ccc(-n2c3ccccc3c3c4c5ccccc5n(-c5cccc([Si](c6ccccc6)(c6ccccc6)c6ccc(-n7c8ccccc8c8ccccc87)cc6)c5)c4ccc32)cc1 ; CC1(C)c2ccccc2-c2c1ccc1c2c2ccccc2n1-c1cccc([Si](c2ccccc2)(c2ccccc2)c2ccc(-n3c4ccccc4c4ccccc43)cc2)c1]

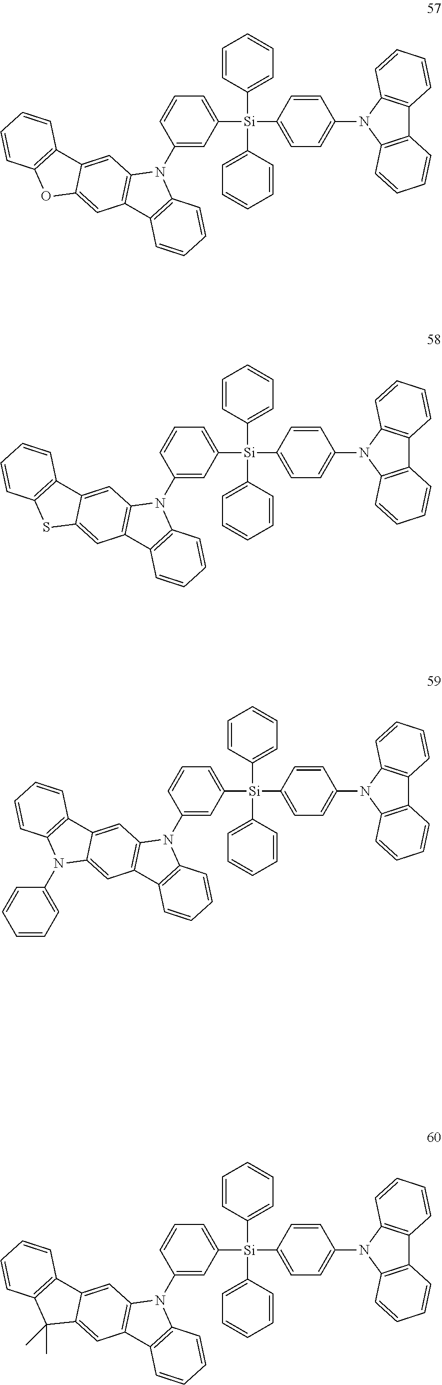 [SMILES: c1ccc([Si](c2ccccc2)(c2ccc(-n3c4ccccc4c4ccccc43)cc2)c2cccc(-n3c4ccccc4c4cc5oc6ccccc6c5cc43)c2)cc1 ; c1ccc([Si](c2ccccc2)(c2ccc(-n3c4ccccc4c4ccccc43)cc2)c2cccc(-n3c4ccccc4c4cc5sc6ccccc6c5cc43)c2)cc1 ; c1ccc(-n2c3ccccc3c3cc4c(cc32)c2ccccc2n4-c2cccc([Si](c3ccccc3)(c3ccccc3)c3ccc(-n4c5ccccc5c5ccccc54)cc3)c2)cc1 ; CC1(C)c2ccccc2-c2cc3c(cc21)c1ccccc1n3-c1cccc([Si](c2ccccc2)(c2ccccc2)c2ccc(-n3c4ccccc4c4ccccc43)cc2)c1]

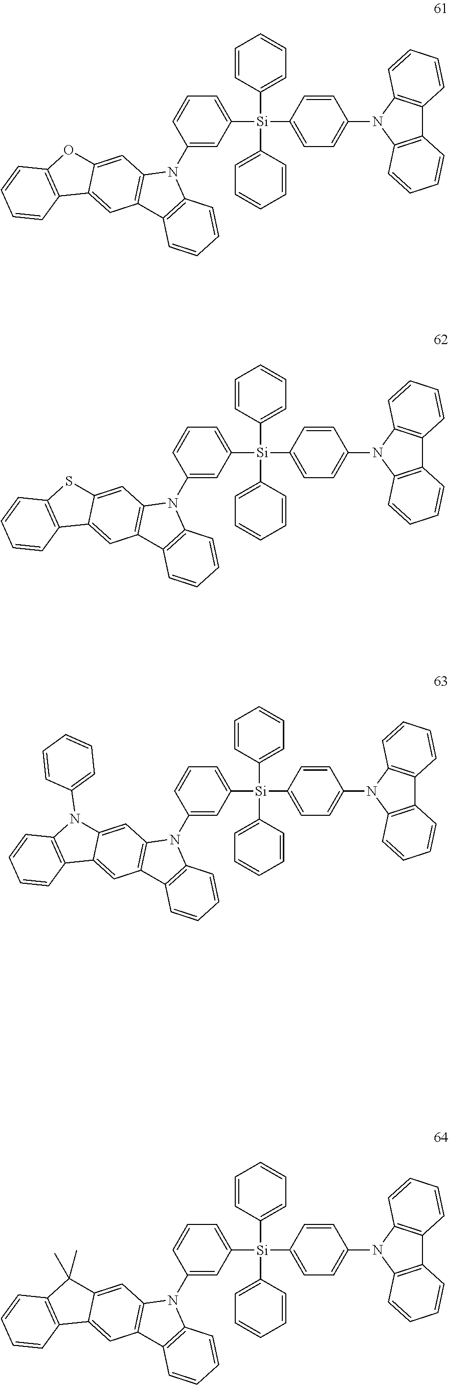 [SMILES: c1ccc([Si](c2ccccc2)(c2ccc(-n3c4ccccc4c4ccccc43)cc2)c2cccc(-n3c4ccccc4c4cc5c(cc43)oc3ccccc35)c2)cc1 ; c1ccc([Si](c2ccccc2)(c2ccc(-n3c4ccccc4c4ccccc43)cc2)c2cccc(-n3c4ccccc4c4cc5c(cc43)sc3ccccc35)c2)cc1 ; c1ccc(-n2c3ccccc3c3cc4c5ccccc5n(-c5cccc([Si](c6ccccc6)(c6ccccc6)c6ccc(-n7c8ccccc8c8ccccc87)cc6)c5)c4cc32)cc1 ; CC1(C)c2ccccc2-c2cc3c4ccccc4n(-c4cccc([Si](c5ccccc5)(c5ccccc5)c5ccc(-n6c7ccccc7c7ccccc76)cc5)c4)c3cc21]

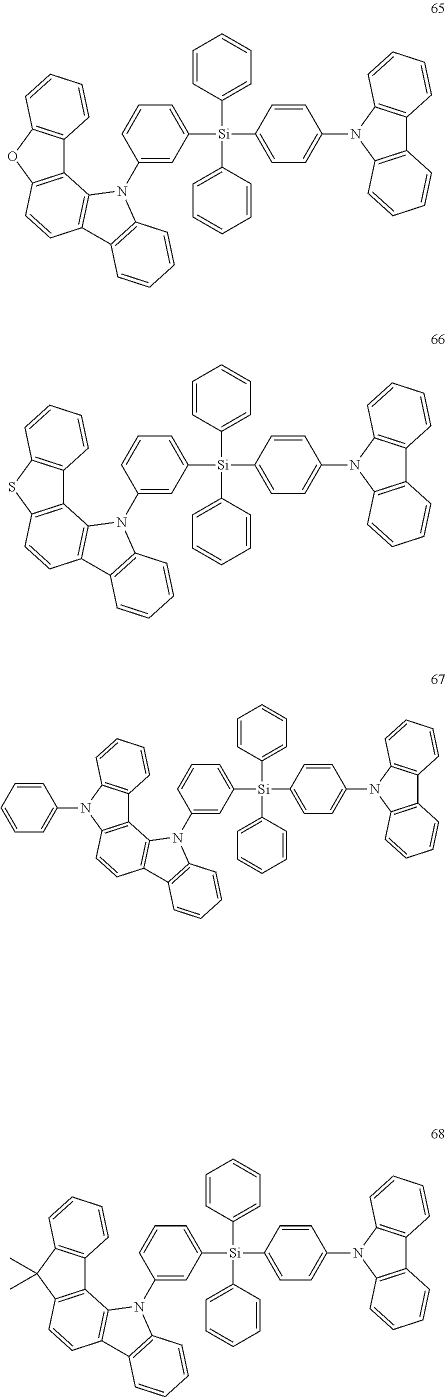 [SMILES: c1ccc([Si](c2ccccc2)(c2ccc(-n3c4ccccc4c4ccccc43)cc2)c2cccc(-n3c4ccccc4c4ccc5oc6ccccc6c5c43)c2)cc1 ; c1ccc([Si](c2ccccc2)(c2ccc(-n3c4ccccc4c4ccccc43)cc2)c2cccc(-n3c4ccccc4c4ccc5sc6ccccc6c5c43)c2)cc1 ; c1ccc(-n2c3ccccc3c3c2ccc2c4ccccc4n(-c4cccc([Si](c5ccccc5)(c5ccccc5)c5ccc(-n6c7ccccc7c7ccccc76)cc5)c4)c23)cc1 ; CC1(C)c2ccccc2-c2c1ccc1c3ccccc3n(-c3cccc([Si](c4ccccc4)(c4ccccc4)c4ccc(-n5c6ccccc6c6ccccc65)cc4)c3)c21]

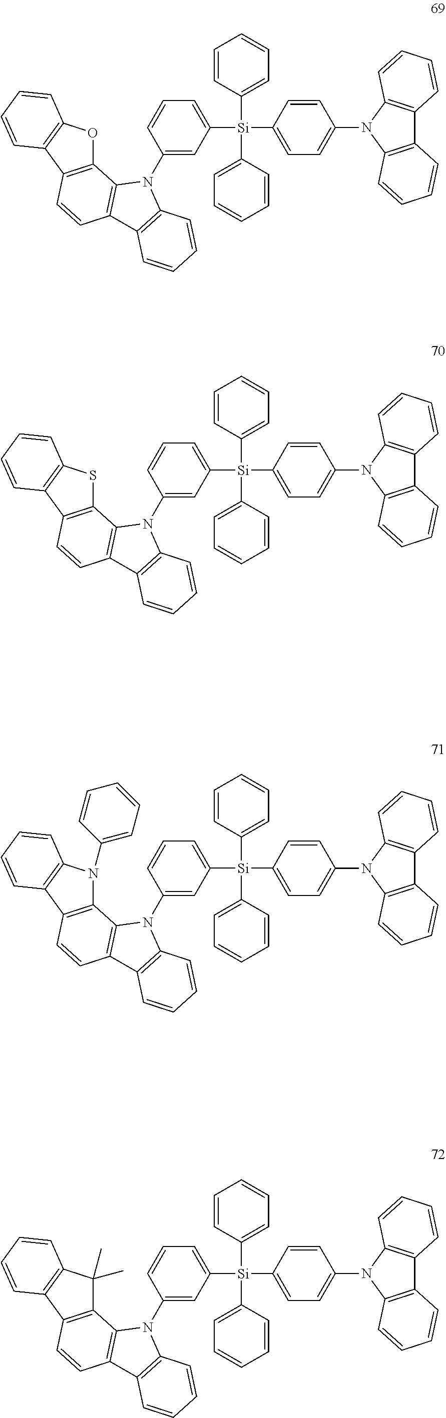 [SMILES: c1ccc([Si](c2ccccc2)(c2ccc(-n3c4ccccc4c4ccccc43)cc2)c2cccc(-n3c4ccccc4c4ccc5c6ccccc6oc5c43)c2)cc1 ; c1ccc([Si](c2ccccc2)(c2ccc(-n3c4ccccc4c4ccccc43)cc2)c2cccc(-n3c4ccccc4c4ccc5c6ccccc6sc5c43)c2)cc1 ; c1ccc(-n2c3ccccc3c3ccc4c5ccccc5n(-c5cccc([Si](c6ccccc6)(c6ccccc6)c6ccc(-n7c8ccccc8c8ccccc87)cc6)c5)c4c32)cc1 ; CC1(C)c2ccccc2-c2ccc3c4ccccc4n(-c4cccc([Si](c5ccccc5)(c5ccccc5)c5ccc(-n6c7ccccc7c7ccccc76)cc5)c4)c3c21]

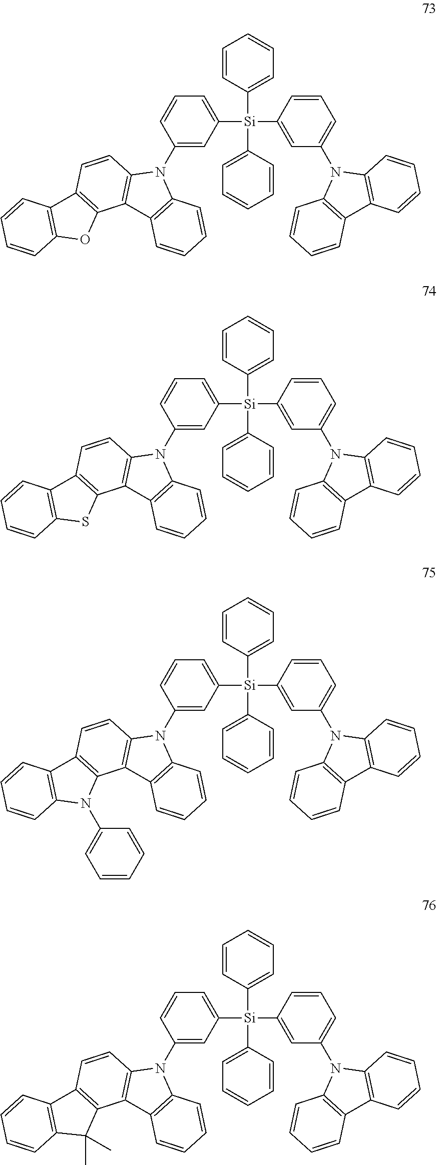 [SMILES: c1ccc([Si](c2ccccc2)(c2cccc(-n3c4ccccc4c4ccccc43)c2)c2cccc(-n3c4ccccc4c4c5oc6ccccc6c5ccc43)c2)cc1 ; c1ccc([Si](c2ccccc2)(c2cccc(-n3c4ccccc4c4ccccc43)c2)c2cccc(-n3c4ccccc4c4c5sc6ccccc6c5ccc43)c2)cc1 ; c1ccc(-n2c3ccccc3c3ccc4c(c5ccccc5n4-c4cccc([Si](c5ccccc5)(c5ccccc5)c5cccc(-n6c7ccccc7c7ccccc76)c5)c4)c32)cc1 ; CC1(C)c2ccccc2-c2ccc3c(c21)c1ccccc1n3-c1cccc([Si](c2ccccc2)(c2ccccc2)c2cccc(-n3c4ccccc4c4ccccc43)c2)c1]

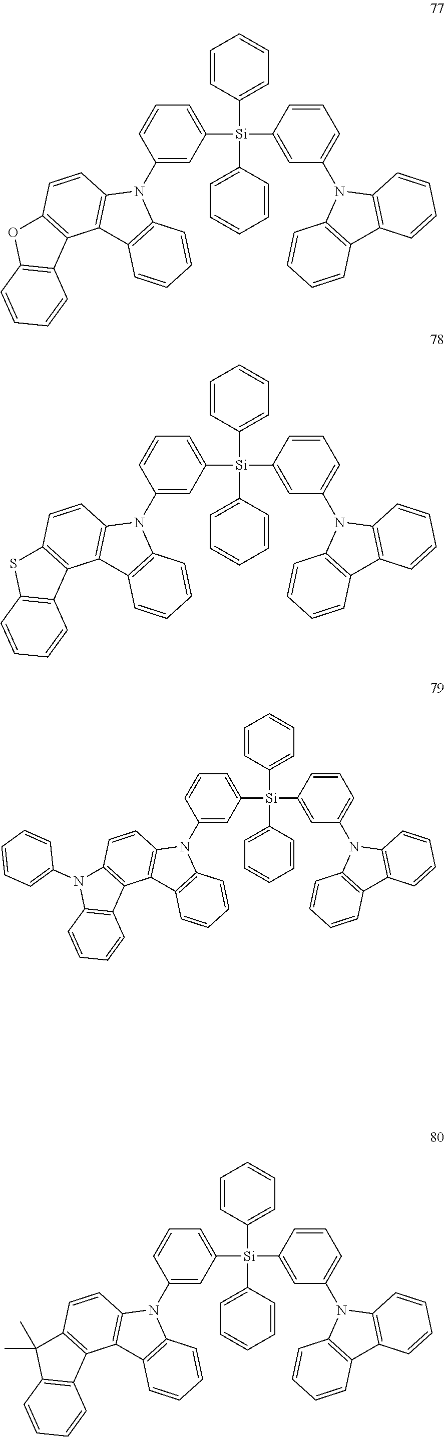 [SMILES: c1ccc([Si](c2ccccc2)(c2cccc(-n3c4ccccc4c4ccccc43)c2)c2cccc(-n3c4ccccc4c4c5c(ccc43)oc3ccccc35)c2)cc1 ; c1ccc([Si](c2ccccc2)(c2cccc(-n3c4ccccc4c4ccccc43)c2)c2cccc(-n3c4ccccc4c4c5c(ccc43)sc3ccccc35)c2)cc1 ; c1ccc(-n2c3ccccc3c3c4c5ccccc5n(-c5cccc([Si](c6ccccc6)(c6ccccc6)c6cccc(-n7c8ccccc8c8ccccc87)c6)c5)c4ccc32)cc1 ; CC1(C)c2ccccc2-c2c1ccc1c2c2ccccc2n1-c1cccc([Si](c2ccccc2)(c2ccccc2)c2cccc(-n3c4ccccc4c4ccccc43)c2)c1]

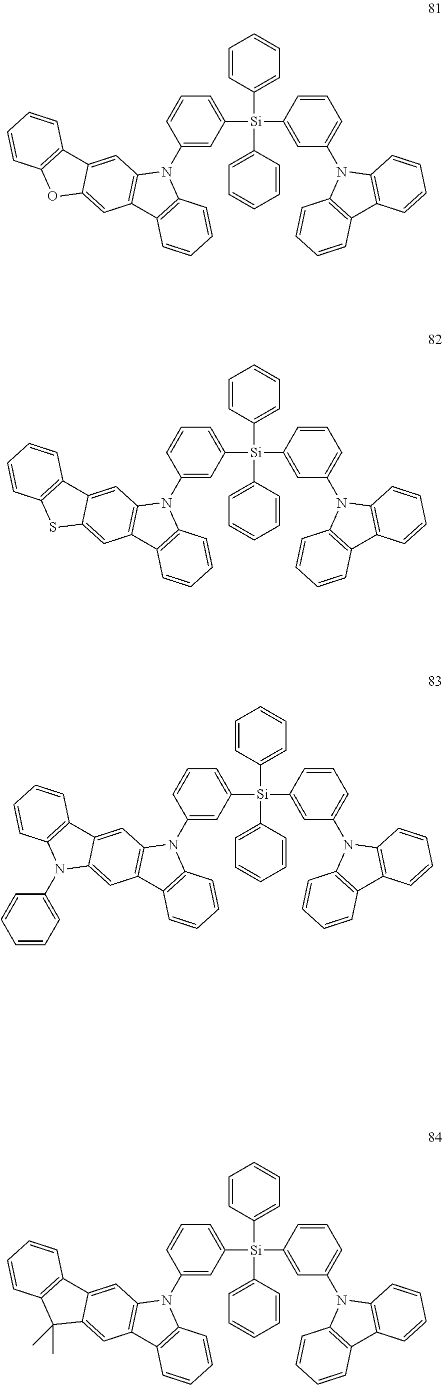 [SMILES: c1ccc([Si](c2ccccc2)(c2cccc(-n3c4ccccc4c4ccccc43)c2)c2cccc(-n3c4ccccc4c4cc5oc6ccccc6c5cc43)c2)cc1 ; c1ccc([Si](c2ccccc2)(c2cccc(-n3c4ccccc4c4ccccc43)c2)c2cccc(-n3c4ccccc4c4cc5sc6ccccc6c5cc43)c2)cc1 ; c1ccc(-n2c3ccccc3c3cc4c(cc32)c2ccccc2n4-c2cccc([Si](c3ccccc3)(c3ccccc3)c3cccc(-n4c5ccccc5c5ccccc54)c3)c2)cc1 ; CC1(C)c2ccccc2-c2cc3c(cc21)c1ccccc1n3-c1cccc([Si](c2ccccc2)(c2ccccc2)c2cccc(-n3c4ccccc4c4ccccc43)c2)c1]

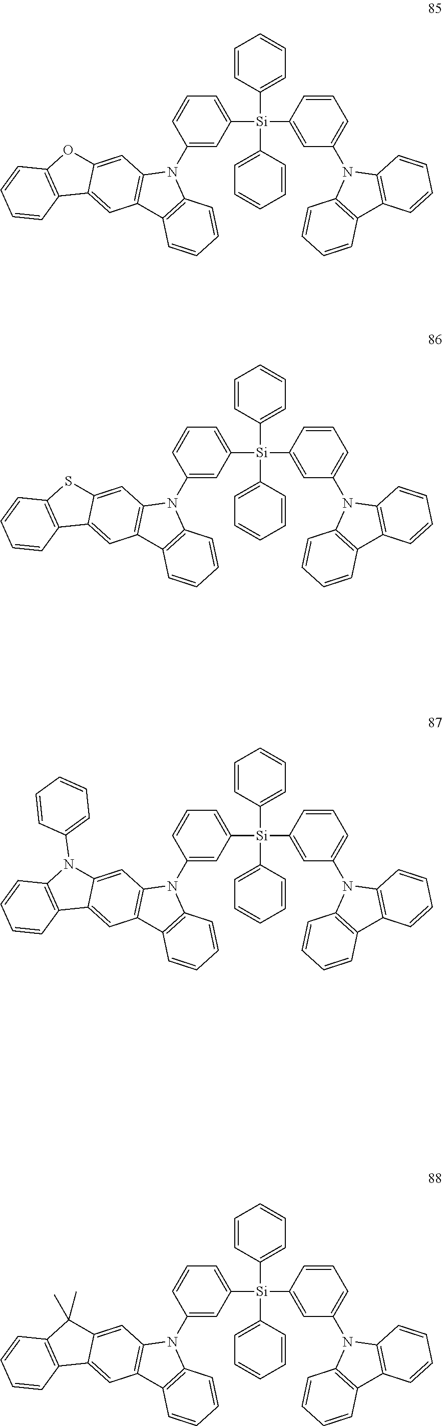 [SMILES: c1ccc([Si](c2ccccc2)(c2cccc(-n3c4ccccc4c4ccccc43)c2)c2cccc(-n3c4ccccc4c4cc5c(cc43)oc3ccccc35)c2)cc1 ; c1ccc([Si](c2ccccc2)(c2cccc(-n3c4ccccc4c4ccccc43)c2)c2cccc(-n3c4ccccc4c4cc5c(cc43)sc3ccccc35)c2)cc1 ; c1ccc(-n2c3ccccc3c3cc4c5ccccc5n(-c5cccc([Si](c6ccccc6)(c6ccccc6)c6cccc(-n7c8ccccc8c8ccccc87)c6)c5)c4cc32)cc1 ; CC1(C)c2ccccc2-c2cc3c4ccccc4n(-c4cccc([Si](c5ccccc5)(c5ccccc5)c5cccc(-n6c7ccccc7c7ccccc76)c5)c4)c3cc21]

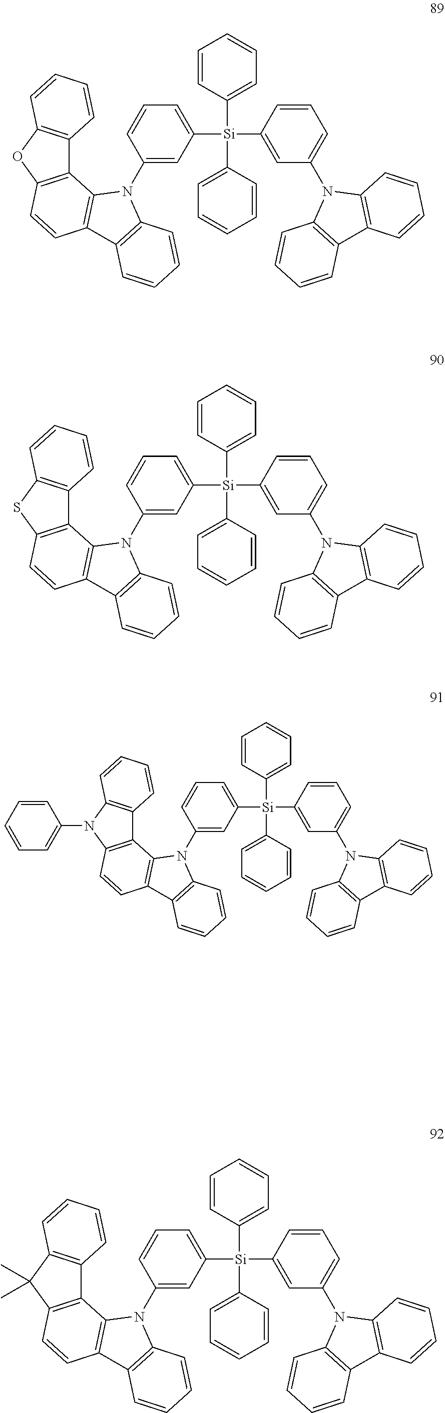 [SMILES: c1ccc([Si](c2ccccc2)(c2cccc(-n3c4ccccc4c4ccccc43)c2)c2cccc(-n3c4ccccc4c4ccc5oc6ccccc6c5c43)c2)cc1 ; c1ccc([Si](c2ccccc2)(c2cccc(-n3c4ccccc4c4ccccc43)c2)c2cccc(-n3c4ccccc4c4ccc5sc6ccccc6c5c43)c2)cc1 ; c1ccc(-n2c3ccccc3c3c2ccc2c4ccccc4n(-c4cccc([Si](c5ccccc5)(c5ccccc5)c5cccc(-n6c7ccccc7c7ccccc76)c5)c4)c23)cc1 ; CC1(C)c2ccccc2-c2c1ccc1c3ccccc3n(-c3cccc([Si](c4ccccc4)(c4ccccc4)c4cccc(-n5c6ccccc6c6ccccc65)c4)c3)c21]

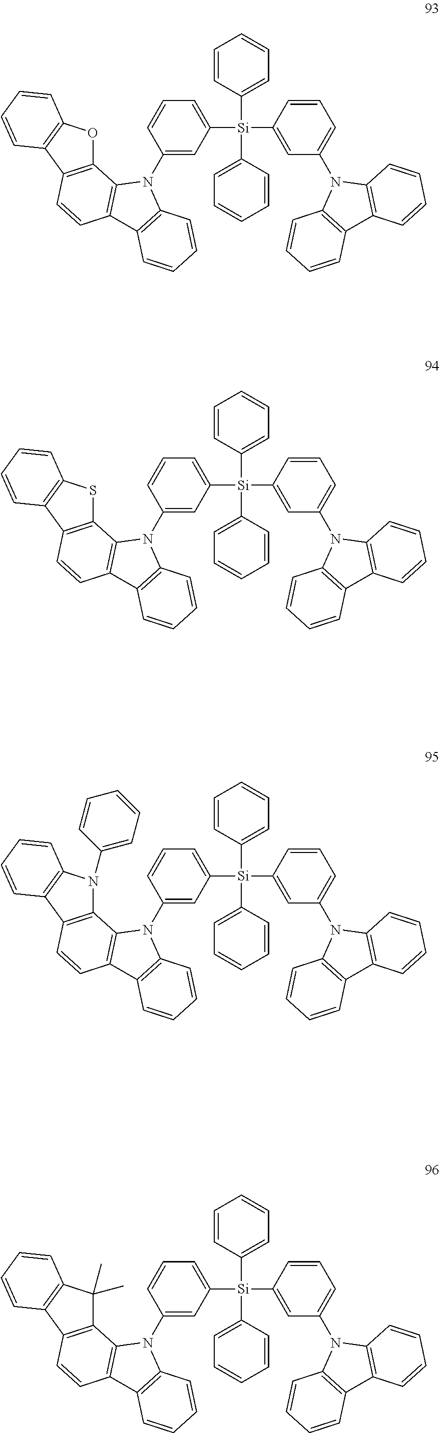 [SMILES: c1ccc([Si](c2ccccc2)(c2cccc(-n3c4ccccc4c4ccccc43)c2)c2cccc(-n3c4ccccc4c4ccc5c6ccccc6oc5c43)c2)cc1 ; c1ccc([Si](c2ccccc2)(c2cccc(-n3c4ccccc4c4ccccc43)c2)c2cccc(-n3c4ccccc4c4ccc5c6ccccc6sc5c43)c2)cc1 ; c1ccc(-n2c3ccccc3c3ccc4c5ccccc5n(-c5cccc([Si](c6ccccc6)(c6ccccc6)c6cccc(-n7c8ccccc8c8ccccc87)c6)c5)c4c32)cc1 ; CC1(C)c2ccccc2-c2ccc3c4ccccc4n(-c4cccc([Si](c5ccccc5)(c5ccccc5)c5cccc(-n6c7ccccc7c7ccccc76)c5)c4)c3c21]

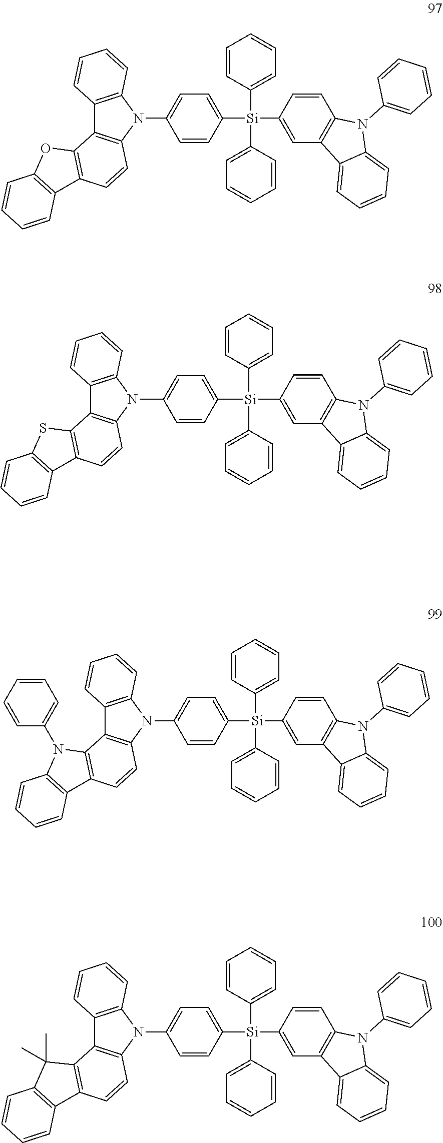 [SMILES: c1ccc(-n2c3ccccc3c3cc([Si](c4ccccc4)(c4ccccc4)c4ccc(-n5c6ccccc6c6c7oc8ccccc8c7ccc65)cc4)ccc32)cc1 ; c1ccc(-n2c3ccccc3c3cc([Si](c4ccccc4)(c4ccccc4)c4ccc(-n5c6ccccc6c6c7sc8ccccc8c7ccc65)cc4)ccc32)cc1 ; c1ccc(-n2c3ccccc3c3cc([Si](c4ccccc4)(c4ccccc4)c4ccc(-n5c6ccccc6c6c5ccc5c7ccccc7n(-c7ccccc7)c56)cc4)ccc32)cc1 ; CC1(C)c2ccccc2-c2ccc3c(c21)c1ccccc1n3-c1ccc([Si](c2ccccc2)(c2ccccc2)c2ccc3c(c2)c2ccccc2n3-c2ccccc2)cc1]

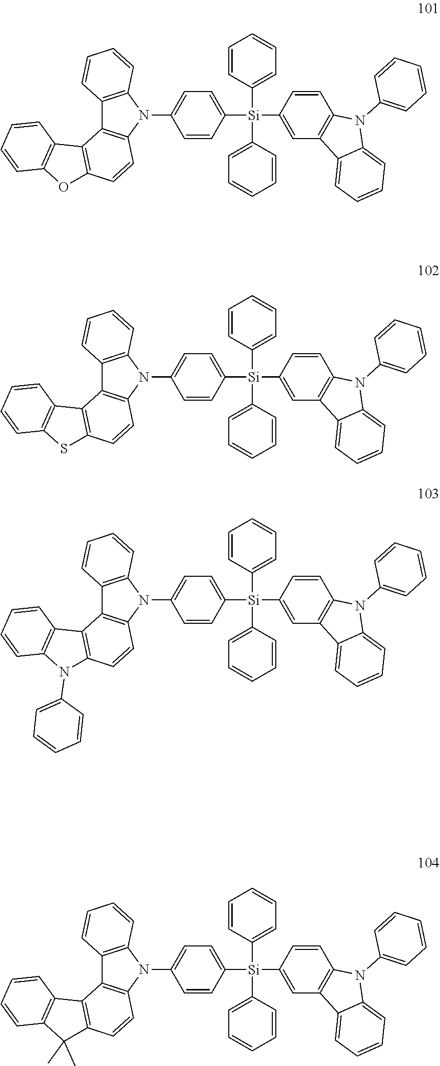 [SMILES: c1ccc(-n2c3ccccc3c3cc([Si](c4ccccc4)(c4ccccc4)c4ccc(-n5c6ccccc6c6c7c(ccc65)oc5ccccc57)cc4)ccc32)cc1 ; c1ccc(-n2c3ccccc3c3cc([Si](c4ccccc4)(c4ccccc4)c4ccc(-n5c6ccccc6c6c7c(ccc65)sc5ccccc57)cc4)ccc32)cc1 ; c1ccc(-n2c3ccccc3c3cc([Si](c4ccccc4)(c4ccccc4)c4ccc(-n5c6ccccc6c6c7c8ccccc8n(-c8ccccc8)c7ccc65)cc4)ccc32)cc1 ; CC1(C)c2ccccc2-c2c1ccc1c2c2ccccc2n1-c1ccc([Si](c2ccccc2)(c2ccccc2)c2ccc3c(c2)c2ccccc2n3-c2ccccc2)cc1]

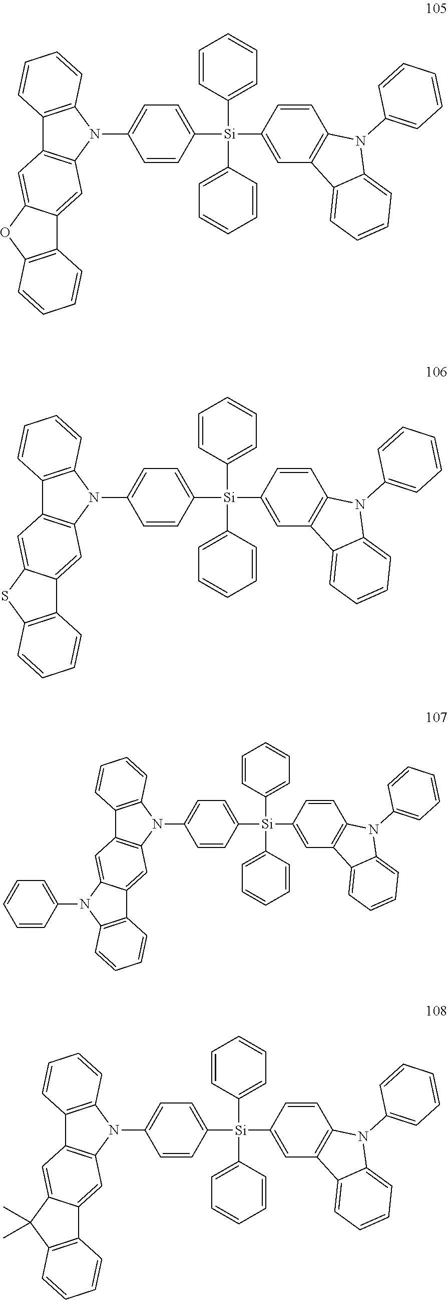 [SMILES: c1ccc(-n2c3ccccc3c3cc([Si](c4ccccc4)(c4ccccc4)c4ccc(-n5c6ccccc6c6cc7oc8ccccc8c7cc65)cc4)ccc32)cc1 ; c1ccc(-n2c3ccccc3c3cc([Si](c4ccccc4)(c4ccccc4)c4ccc(-n5c6ccccc6c6cc7sc8ccccc8c7cc65)cc4)ccc32)cc1 ; c1ccc(-n2c3ccccc3c3cc([Si](c4ccccc4)(c4ccccc4)c4ccc(-n5c6ccccc6c6cc7c(cc65)c5ccccc5n7-c5ccccc5)cc4)ccc32)cc1 ; CC1(C)c2ccccc2-c2cc3c(cc21)c1ccccc1n3-c1ccc([Si](c2ccccc2)(c2ccccc2)c2ccc3c(c2)c2ccccc2n3-c2ccccc2)cc1]

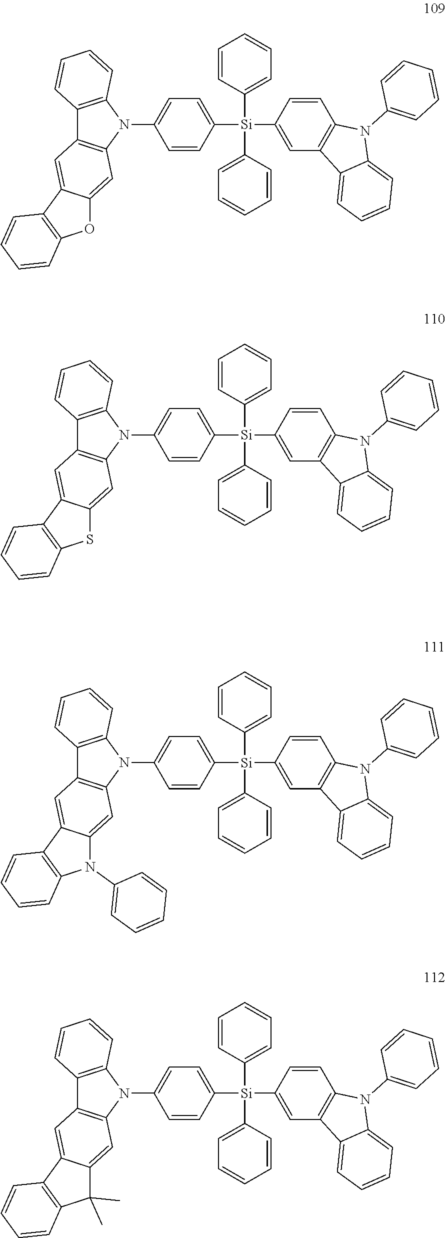 [SMILES: c1ccc(-n2c3ccccc3c3cc([Si](c4ccccc4)(c4ccccc4)c4ccc(-n5c6ccccc6c6cc7c(cc65)oc5ccccc57)cc4)ccc32)cc1 ; c1ccc(-n2c3ccccc3c3cc([Si](c4ccccc4)(c4ccccc4)c4ccc(-n5c6ccccc6c6cc7c(cc65)sc5ccccc57)cc4)ccc32)cc1 ; c1ccc(-n2c3ccccc3c3cc([Si](c4ccccc4)(c4ccccc4)c4ccc(-n5c6ccccc6c6cc7c8ccccc8n(-c8ccccc8)c7cc65)cc4)ccc32)cc1 ; CC1(C)c2ccccc2-c2cc3c4ccccc4n(-c4ccc([Si](c5ccccc5)(c5ccccc5)c5ccc6c(c5)c5ccccc5n6-c5ccccc5)cc4)c3cc21]

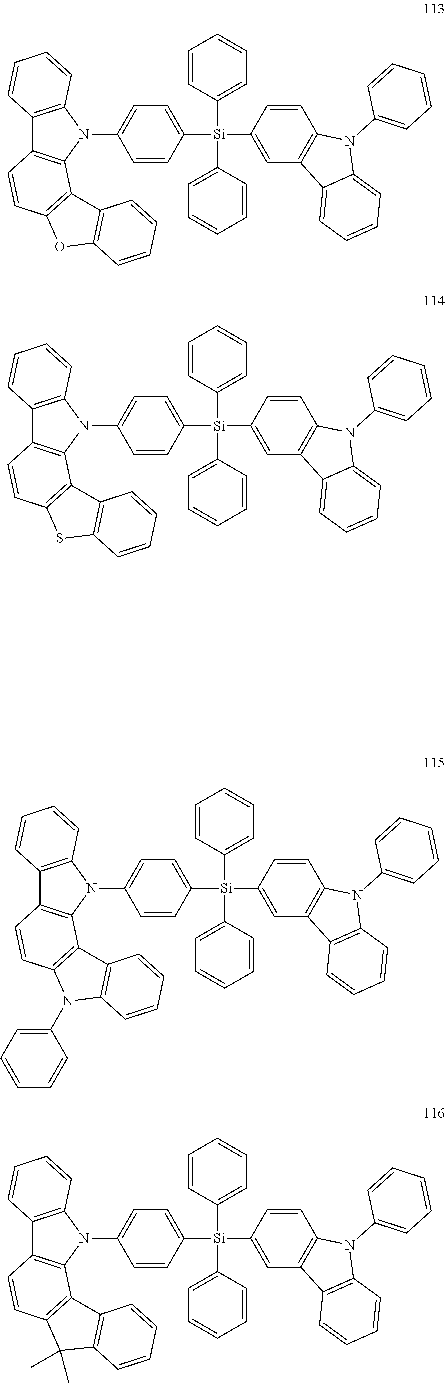 [SMILES: c1ccc(-n2c3ccccc3c3cc([Si](c4ccccc4)(c4ccccc4)c4ccc(-n5c6ccccc6c6ccc7oc8ccccc8c7c65)cc4)ccc32)cc1 ; c1ccc(-n2c3ccccc3c3cc([Si](c4ccccc4)(c4ccccc4)c4ccc(-n5c6ccccc6c6ccc7sc8ccccc8c7c65)cc4)ccc32)cc1 ; c1ccc(-n2c3ccccc3c3cc([Si](c4ccccc4)(c4ccccc4)c4ccc(-n5c6ccccc6c6ccc7c(c8ccccc8n7-c7ccccc7)c65)cc4)ccc32)cc1 ; CC1(C)c2ccccc2-c2c1ccc1c3ccccc3n(-c3ccc([Si](c4ccccc4)(c4ccccc4)c4ccc5c(c4)c4ccccc4n5-c4ccccc4)cc3)c21]

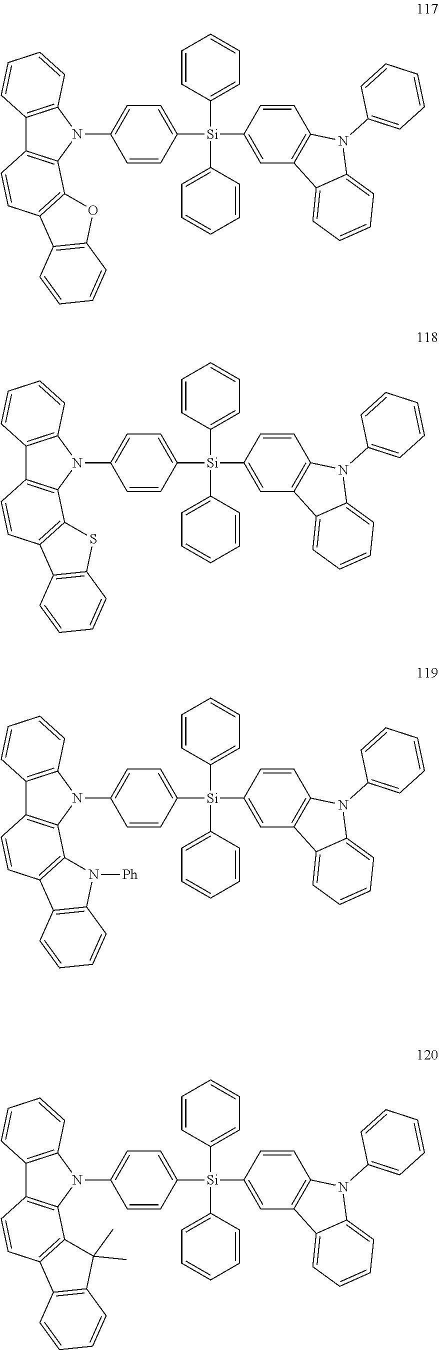 [SMILES: c1ccc(-n2c3ccccc3c3cc([Si](c4ccccc4)(c4ccccc4)c4ccc(-n5c6ccccc6c6ccc7c8ccccc8oc7c65)cc4)ccc32)cc1 ; c1ccc(-n2c3ccccc3c3cc([Si](c4ccccc4)(c4ccccc4)c4ccc(-n5c6ccccc6c6ccc7c8ccccc8sc7c65)cc4)ccc32)cc1 ; c1ccc(-n2c3ccccc3c3cc([Si](c4ccccc4)(c4ccccc4)c4ccc(-n5c6ccccc6c6ccc7c8ccccc8n(-c8ccccc8)c7c65)cc4)ccc32)cc1 ; CC1(C)c2ccccc2-c2ccc3c4ccccc4n(-c4ccc([Si](c5ccccc5)(c5ccccc5)c5ccc6c(c5)c5ccccc5n6-c5ccccc5)cc4)c3c21]

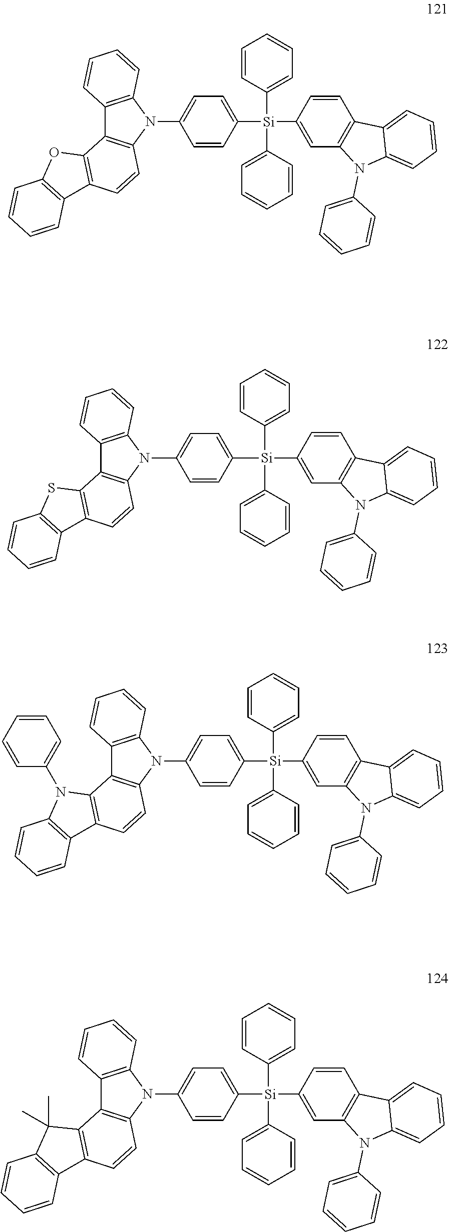 [SMILES: c1ccc(-n2c3ccccc3c3ccc([Si](c4ccccc4)(c4ccccc4)c4ccc(-n5c6ccccc6c6c7oc8ccccc8c7ccc65)cc4)cc32)cc1 ; c1ccc(-n2c3ccccc3c3ccc([Si](c4ccccc4)(c4ccccc4)c4ccc(-n5c6ccccc6c6c7sc8ccccc8c7ccc65)cc4)cc32)cc1 ; c1ccc(-n2c3ccccc3c3ccc([Si](c4ccccc4)(c4ccccc4)c4ccc(-n5c6ccccc6c6c5ccc5c7ccccc7n(-c7ccccc7)c56)cc4)cc32)cc1 ; CC1(C)c2ccccc2-c2ccc3c(c21)c1ccccc1n3-c1ccc([Si](c2ccccc2)(c2ccccc2)c2ccc3c4ccccc4n(-c4ccccc4)c3c2)cc1]

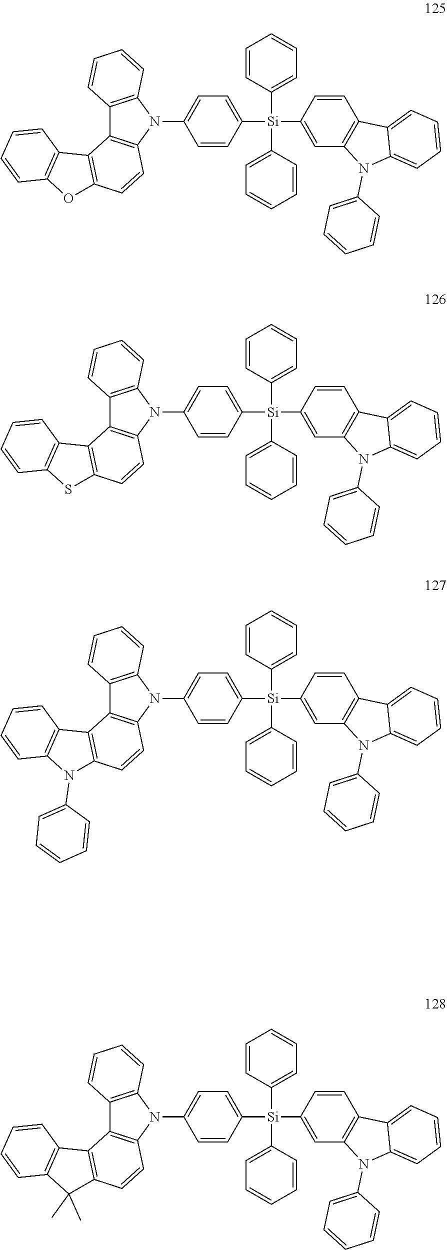 [SMILES: c1ccc(-n2c3ccccc3c3ccc([Si](c4ccccc4)(c4ccccc4)c4ccc(-n5c6ccccc6c6c7c(ccc65)oc5ccccc57)cc4)cc32)cc1 ; c1ccc(-n2c3ccccc3c3ccc([Si](c4ccccc4)(c4ccccc4)c4ccc(-n5c6ccccc6c6c7c(ccc65)sc5ccccc57)cc4)cc32)cc1 ; c1ccc(-n2c3ccccc3c3ccc([Si](c4ccccc4)(c4ccccc4)c4ccc(-n5c6ccccc6c6c7c8ccccc8n(-c8ccccc8)c7ccc65)cc4)cc32)cc1 ; CC1(C)c2ccccc2-c2c1ccc1c2c2ccccc2n1-c1ccc([Si](c2ccccc2)(c2ccccc2)c2ccc3c4ccccc4n(-c4ccccc4)c3c2)cc1]

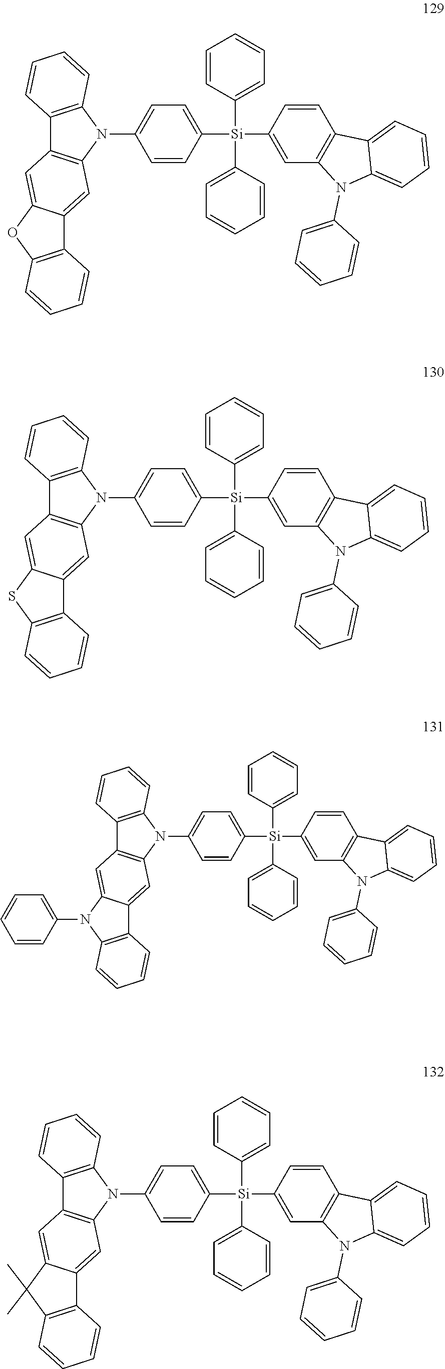 [SMILES: c1ccc(-n2c3ccccc3c3ccc([Si](c4ccccc4)(c4ccccc4)c4ccc(-n5c6ccccc6c6cc7oc8ccccc8c7cc65)cc4)cc32)cc1 ; c1ccc(-n2c3ccccc3c3ccc([Si](c4ccccc4)(c4ccccc4)c4ccc(-n5c6ccccc6c6cc7sc8ccccc8c7cc65)cc4)cc32)cc1 ; c1ccc(-n2c3ccccc3c3ccc([Si](c4ccccc4)(c4ccccc4)c4ccc(-n5c6ccccc6c6cc7c(cc65)c5ccccc5n7-c5ccccc5)cc4)cc32)cc1 ; CC1(C)c2ccccc2-c2cc3c(cc21)c1ccccc1n3-c1ccc([Si](c2ccccc2)(c2ccccc2)c2ccc3c4ccccc4n(-c4ccccc4)c3c2)cc1]

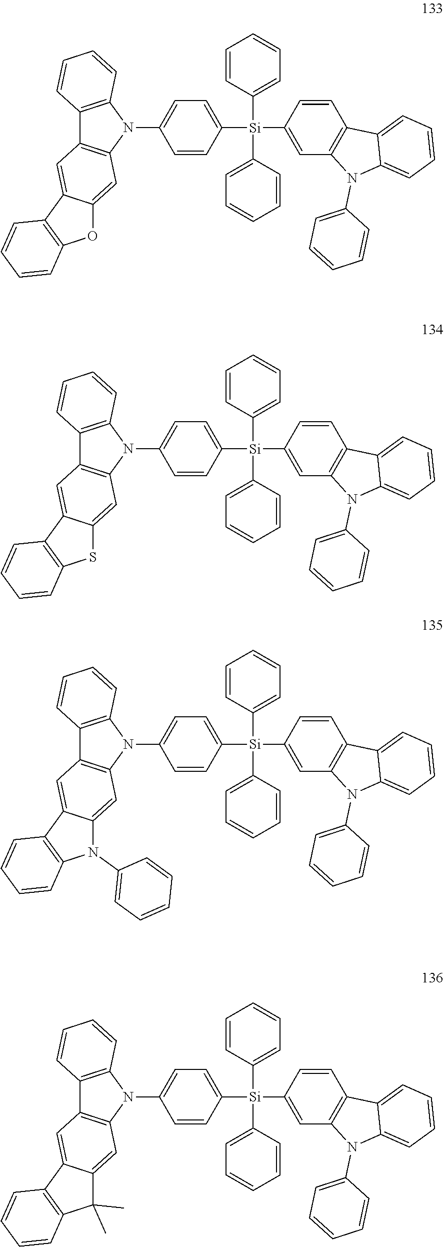 [SMILES: c1ccc(-n2c3ccccc3c3ccc([Si](c4ccccc4)(c4ccccc4)c4ccc(-n5c6ccccc6c6cc7c(cc65)oc5ccccc57)cc4)cc32)cc1 ; c1ccc(-n2c3ccccc3c3ccc([Si](c4ccccc4)(c4ccccc4)c4ccc(-n5c6ccccc6c6cc7c(cc65)sc5ccccc57)cc4)cc32)cc1 ; c1ccc(-n2c3ccccc3c3ccc([Si](c4ccccc4)(c4ccccc4)c4ccc(-n5c6ccccc6c6cc7c8ccccc8n(-c8ccccc8)c7cc65)cc4)cc32)cc1 ; CC1(C)c2ccccc2-c2cc3c4ccccc4n(-c4ccc([Si](c5ccccc5)(c5ccccc5)c5ccc6c7ccccc7n(-c7ccccc7)c6c5)cc4)c3cc21]

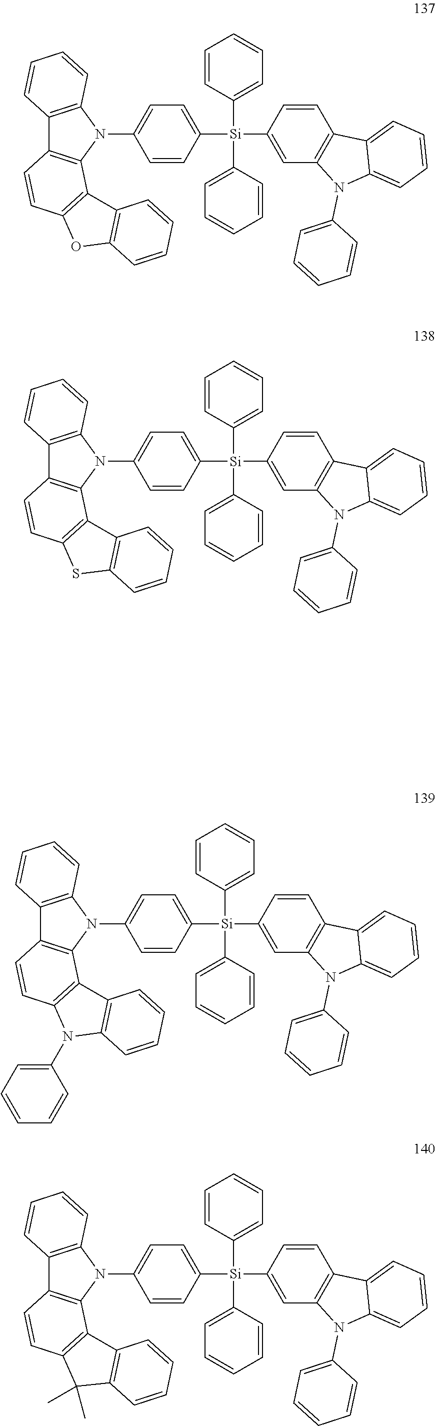 [SMILES: c1ccc(-n2c3ccccc3c3ccc([Si](c4ccccc4)(c4ccccc4)c4ccc(-n5c6ccccc6c6ccc7oc8ccccc8c7c65)cc4)cc32)cc1 ; c1ccc(-n2c3ccccc3c3ccc([Si](c4ccccc4)(c4ccccc4)c4ccc(-n5c6ccccc6c6ccc7sc8ccccc8c7c65)cc4)cc32)cc1 ; c1ccc(-n2c3ccccc3c3ccc([Si](c4ccccc4)(c4ccccc4)c4ccc(-n5c6ccccc6c6ccc7c(c8ccccc8n7-c7ccccc7)c65)cc4)cc32)cc1 ; CC1(C)c2ccccc2-c2c1ccc1c3ccccc3n(-c3ccc([Si](c4ccccc4)(c4ccccc4)c4ccc5c6ccccc6n(-c6ccccc6)c5c4)cc3)c21]

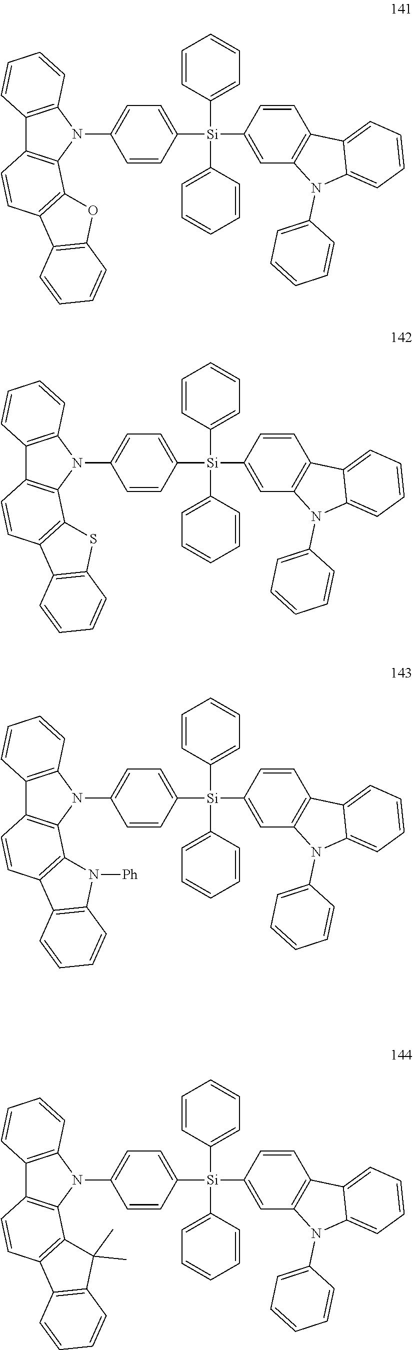 [SMILES: c1ccc(-n2c3ccccc3c3ccc([Si](c4ccccc4)(c4ccccc4)c4ccc(-n5c6ccccc6c6ccc7c8ccccc8oc7c65)cc4)cc32)cc1 ; c1ccc(-n2c3ccccc3c3ccc([Si](c4ccccc4)(c4ccccc4)c4ccc(-n5c6ccccc6c6ccc7c8ccccc8sc7c65)cc4)cc32)cc1 ; c1ccc(-n2c3ccccc3c3ccc([Si](c4ccccc4)(c4ccccc4)c4ccc(-n5c6ccccc6c6ccc7c8ccccc8n(-c8ccccc8)c7c65)cc4)cc32)cc1 ; CC1(C)c2ccccc2-c2ccc3c4ccccc4n(-c4ccc([Si](c5ccccc5)(c5ccccc5)c5ccc6c7ccccc7n(-c7ccccc7)c6c5)cc4)c3c21]

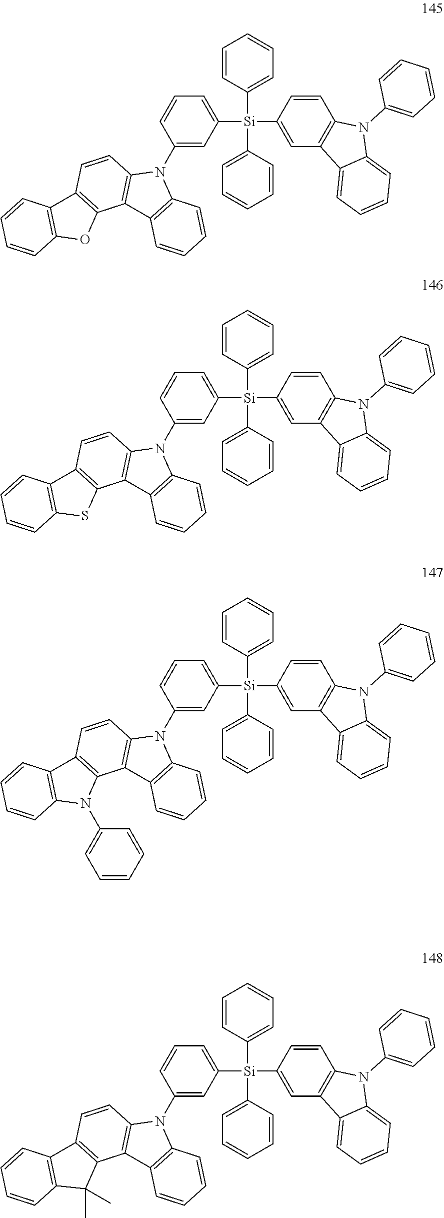 [SMILES: c1ccc(-n2c3ccccc3c3cc([Si](c4ccccc4)(c4ccccc4)c4cccc(-n5c6ccccc6c6c7oc8ccccc8c7ccc65)c4)ccc32)cc1 ; c1ccc(-n2c3ccccc3c3cc([Si](c4ccccc4)(c4ccccc4)c4cccc(-n5c6ccccc6c6c7sc8ccccc8c7ccc65)c4)ccc32)cc1 ; c1ccc(-n2c3ccccc3c3cc([Si](c4ccccc4)(c4ccccc4)c4cccc(-n5c6ccccc6c6c5ccc5c7ccccc7n(-c7ccccc7)c56)c4)ccc32)cc1 ; CC1(C)c2ccccc2-c2ccc3c(c21)c1ccccc1n3-c1cccc([Si](c2ccccc2)(c2ccccc2)c2ccc3c(c2)c2ccccc2n3-c2ccccc2)c1]

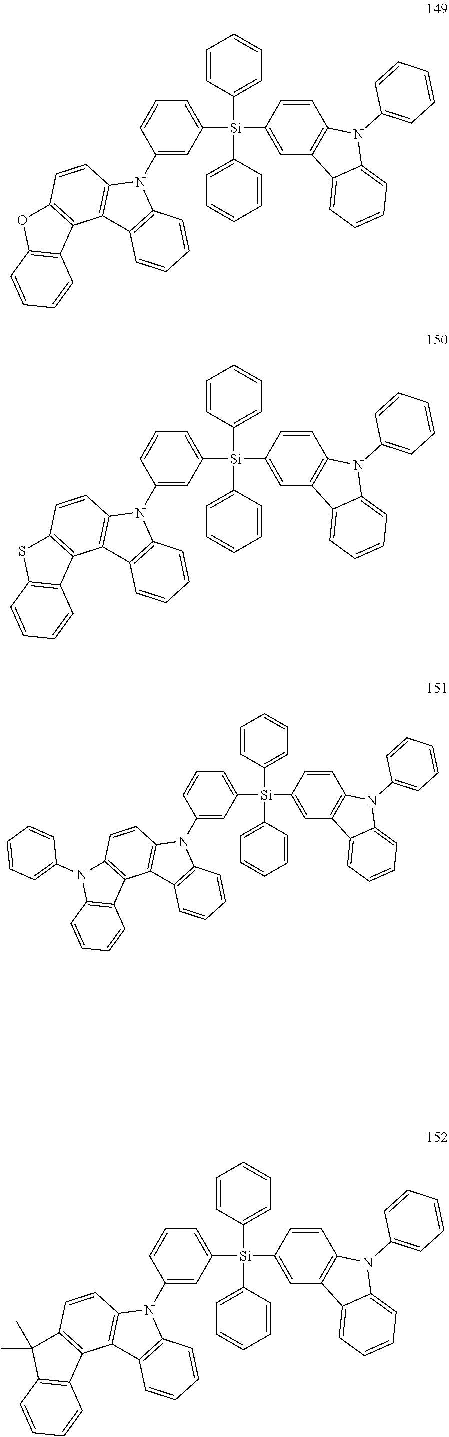 [SMILES: c1ccc(-n2c3ccccc3c3cc([Si](c4ccccc4)(c4ccccc4)c4cccc(-n5c6ccccc6c6c7c(ccc65)oc5ccccc57)c4)ccc32)cc1 ; c1ccc(-n2c3ccccc3c3cc([Si](c4ccccc4)(c4ccccc4)c4cccc(-n5c6ccccc6c6c7c(ccc65)sc5ccccc57)c4)ccc32)cc1 ; c1ccc(-n2c3ccccc3c3cc([Si](c4ccccc4)(c4ccccc4)c4cccc(-n5c6ccccc6c6c7c8ccccc8n(-c8ccccc8)c7ccc65)c4)ccc32)cc1 ; CC1(C)c2ccccc2-c2c1ccc1c2c2ccccc2n1-c1cccc([Si](c2ccccc2)(c2ccccc2)c2ccc3c(c2)c2ccccc2n3-c2ccccc2)c1]

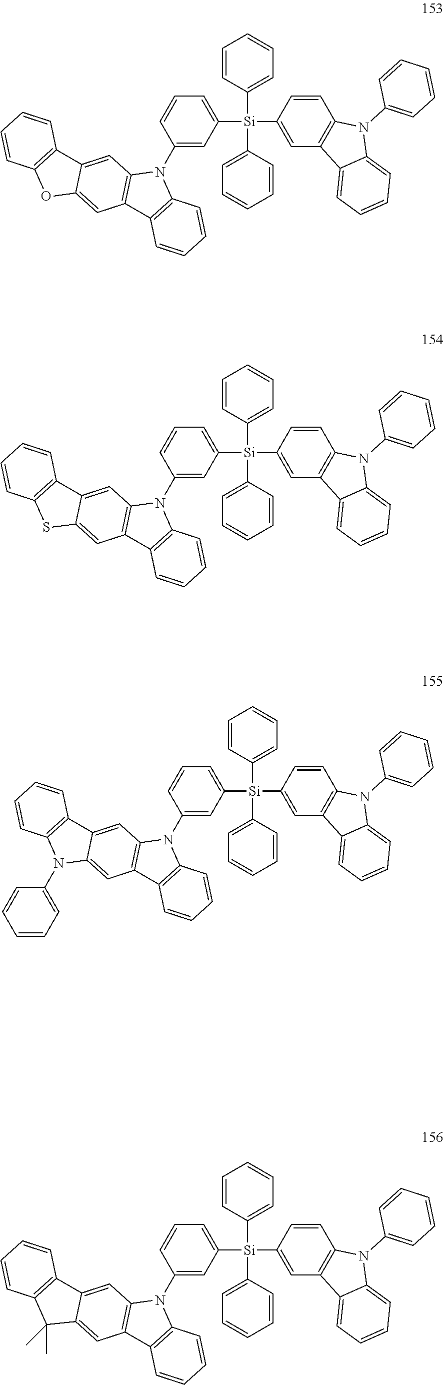 [SMILES: c1ccc(-n2c3ccccc3c3cc([Si](c4ccccc4)(c4ccccc4)c4cccc(-n5c6ccccc6c6cc7oc8ccccc8c7cc65)c4)ccc32)cc1 ; c1ccc(-n2c3ccccc3c3cc([Si](c4ccccc4)(c4ccccc4)c4cccc(-n5c6ccccc6c6cc7sc8ccccc8c7cc65)c4)ccc32)cc1 ; c1ccc(-n2c3ccccc3c3cc([Si](c4ccccc4)(c4ccccc4)c4cccc(-n5c6ccccc6c6cc7c(cc65)c5ccccc5n7-c5ccccc5)c4)ccc32)cc1 ; CC1(C)c2ccccc2-c2cc3c(cc21)c1ccccc1n3-c1cccc([Si](c2ccccc2)(c2ccccc2)c2ccc3c(c2)c2ccccc2n3-c2ccccc2)c1]

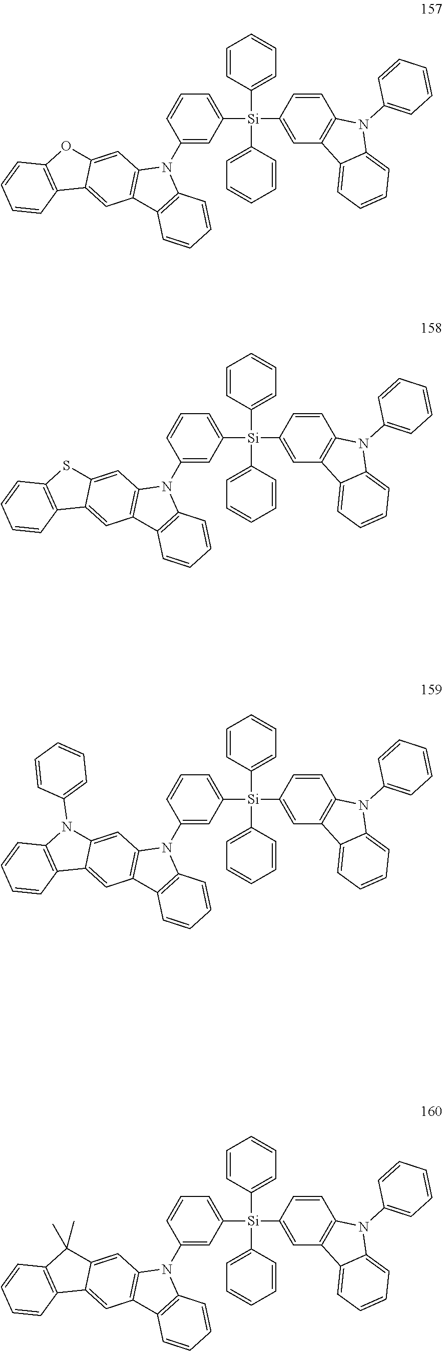 [SMILES: c1ccc(-n2c3ccccc3c3cc([Si](c4ccccc4)(c4ccccc4)c4cccc(-n5c6ccccc6c6cc7c(cc65)oc5ccccc57)c4)ccc32)cc1 ; c1ccc(-n2c3ccccc3c3cc([Si](c4ccccc4)(c4ccccc4)c4cccc(-n5c6ccccc6c6cc7c(cc65)sc5ccccc57)c4)ccc32)cc1 ; c1ccc(-n2c3ccccc3c3cc([Si](c4ccccc4)(c4ccccc4)c4cccc(-n5c6ccccc6c6cc7c8ccccc8n(-c8ccccc8)c7cc65)c4)ccc32)cc1 ; CC1(C)c2ccccc2-c2cc3c4ccccc4n(-c4cccc([Si](c5ccccc5)(c5ccccc5)c5ccc6c(c5)c5ccccc5n6-c5ccccc5)c4)c3cc21]

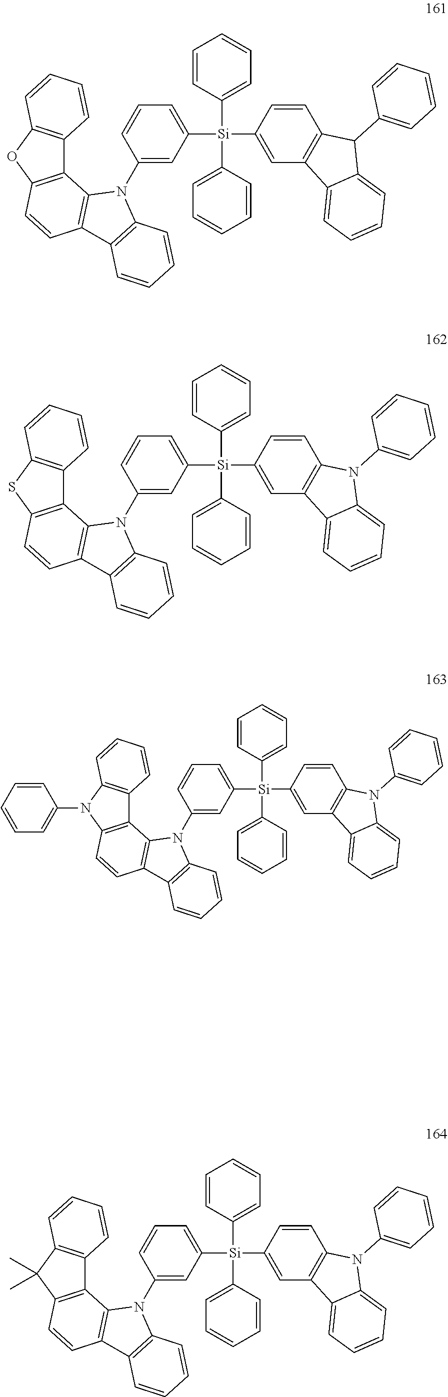 [SMILES: c1ccc(C2c3ccccc3-c3cc([Si](c4ccccc4)(c4ccccc4)c4cccc(-n5c6ccccc6c6ccc7oc8ccccc8c7c65)c4)ccc32)cc1 ; c1ccc(-n2c3ccccc3c3cc([Si](c4ccccc4)(c4ccccc4)c4cccc(-n5c6ccccc6c6ccc7sc8ccccc8c7c65)c4)ccc32)cc1 ; c1ccc(-n2c3ccccc3c3cc([Si](c4ccccc4)(c4ccccc4)c4cccc(-n5c6ccccc6c6ccc7c(c8ccccc8n7-c7ccccc7)c65)c4)ccc32)cc1 ; CC1(C)c2ccccc2-c2c1ccc1c3ccccc3n(-c3cccc([Si](c4ccccc4)(c4ccccc4)c4ccc5c(c4)c4ccccc4n5-c4ccccc4)c3)c21]

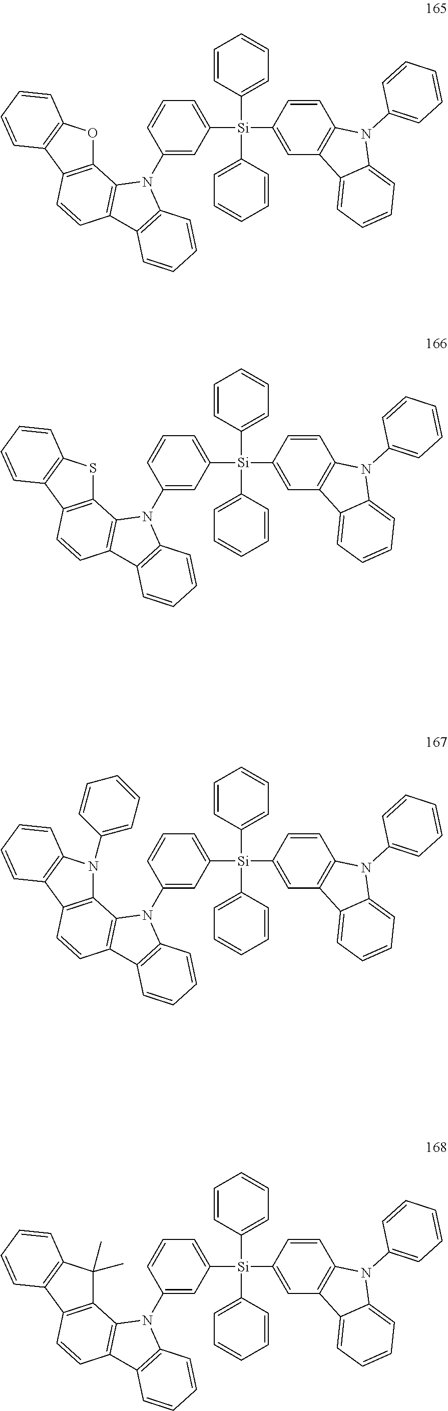 [SMILES: c1ccc(-n2c3ccccc3c3cc([Si](c4ccccc4)(c4ccccc4)c4cccc(-n5c6ccccc6c6ccc7c8ccccc8oc7c65)c4)ccc32)cc1 ; c1ccc(-n2c3ccccc3c3cc([Si](c4ccccc4)(c4ccccc4)c4cccc(-n5c6ccccc6c6ccc7c8ccccc8sc7c65)c4)ccc32)cc1 ; c1ccc(-n2c3ccccc3c3cc([Si](c4ccccc4)(c4ccccc4)c4cccc(-n5c6ccccc6c6ccc7c8ccccc8n(-c8ccccc8)c7c65)c4)ccc32)cc1 ; CC1(C)c2ccccc2-c2ccc3c4ccccc4n(-c4cccc([Si](c5ccccc5)(c5ccccc5)c5ccc6c(c5)c5ccccc5n6-c5ccccc5)c4)c3c21]

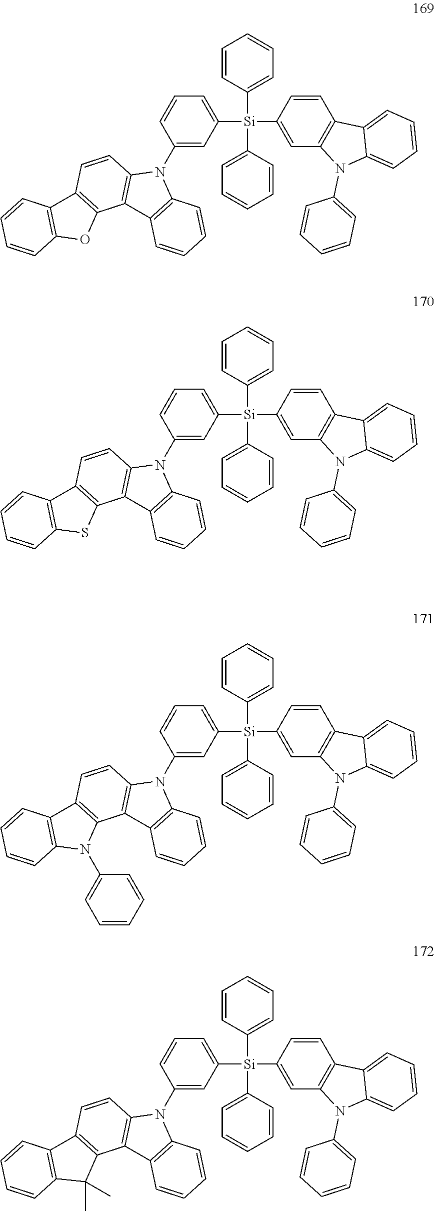 [SMILES: c1ccc(-n2c3ccccc3c3ccc([Si](c4ccccc4)(c4ccccc4)c4cccc(-n5c6ccccc6c6c7oc8ccccc8c7ccc65)c4)cc32)cc1 ; c1ccc(-n2c3ccccc3c3ccc([Si](c4ccccc4)(c4ccccc4)c4cccc(-n5c6ccccc6c6c7sc8ccccc8c7ccc65)c4)cc32)cc1 ; c1ccc(-n2c3ccccc3c3ccc([Si](c4ccccc4)(c4ccccc4)c4cccc(-n5c6ccccc6c6c5ccc5c7ccccc7n(-c7ccccc7)c56)c4)cc32)cc1 ; CC1(C)c2ccccc2-c2ccc3c(c21)c1ccccc1n3-c1cccc([Si](c2ccccc2)(c2ccccc2)c2ccc3c4ccccc4n(-c4ccccc4)c3c2)c1]

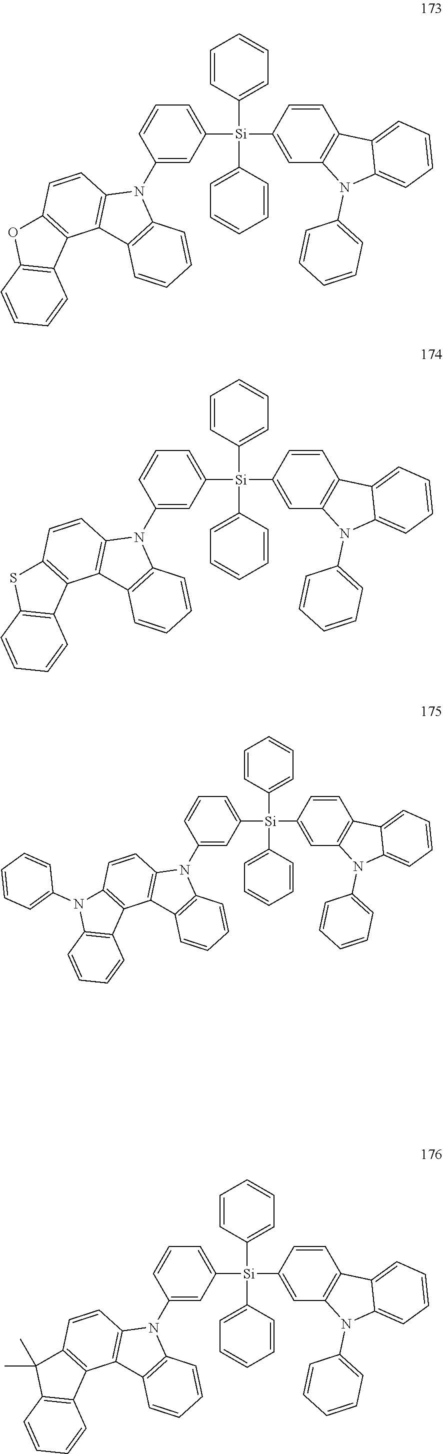 [SMILES: c1ccc(-n2c3ccccc3c3ccc([Si](c4ccccc4)(c4ccccc4)c4cccc(-n5c6ccccc6c6c7c(ccc65)oc5ccccc57)c4)cc32)cc1 ; c1ccc(-n2c3ccccc3c3ccc([Si](c4ccccc4)(c4ccccc4)c4cccc(-n5c6ccccc6c6c7c(ccc65)sc5ccccc57)c4)cc32)cc1 ; c1ccc(-n2c3ccccc3c3ccc([Si](c4ccccc4)(c4ccccc4)c4cccc(-n5c6ccccc6c6c7c8ccccc8n(-c8ccccc8)c7ccc65)c4)cc32)cc1 ; CC1(C)c2ccccc2-c2c1ccc1c2c2ccccc2n1-c1cccc([Si](c2ccccc2)(c2ccccc2)c2ccc3c4ccccc4n(-c4ccccc4)c3c2)c1]

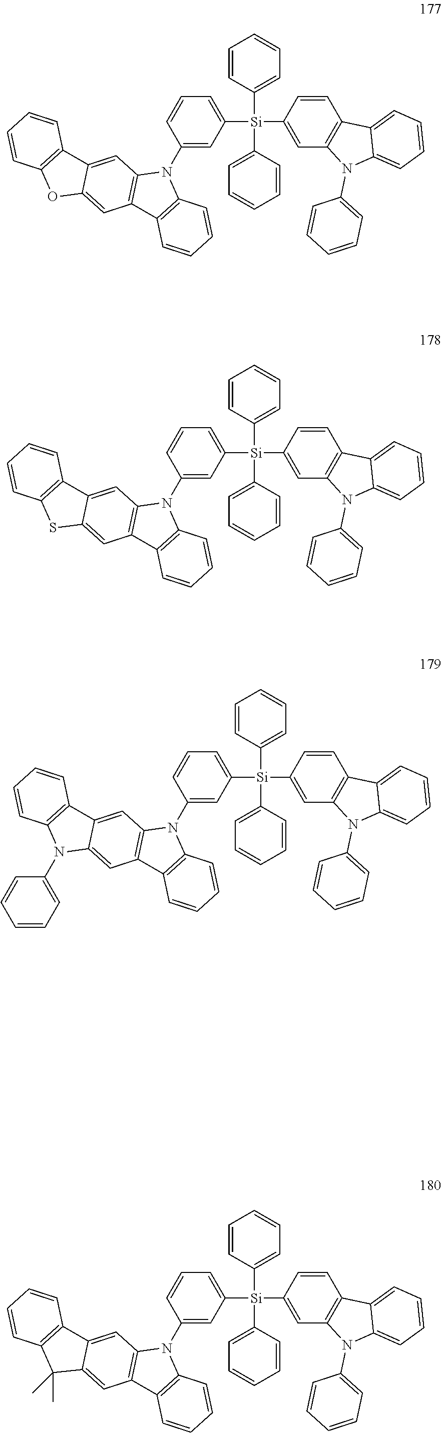 [SMILES: c1ccc(-n2c3ccccc3c3ccc([Si](c4ccccc4)(c4ccccc4)c4cccc(-n5c6ccccc6c6cc7oc8ccccc8c7cc65)c4)cc32)cc1 ; c1ccc(-n2c3ccccc3c3ccc([Si](c4ccccc4)(c4ccccc4)c4cccc(-n5c6ccccc6c6cc7sc8ccccc8c7cc65)c4)cc32)cc1 ; c1ccc(-n2c3ccccc3c3ccc([Si](c4ccccc4)(c4ccccc4)c4cccc(-n5c6ccccc6c6cc7c(cc65)c5ccccc5n7-c5ccccc5)c4)cc32)cc1 ; CC1(C)c2ccccc2-c2cc3c(cc21)c1ccccc1n3-c1cccc([Si](c2ccccc2)(c2ccccc2)c2ccc3c4ccccc4n(-c4ccccc4)c3c2)c1]

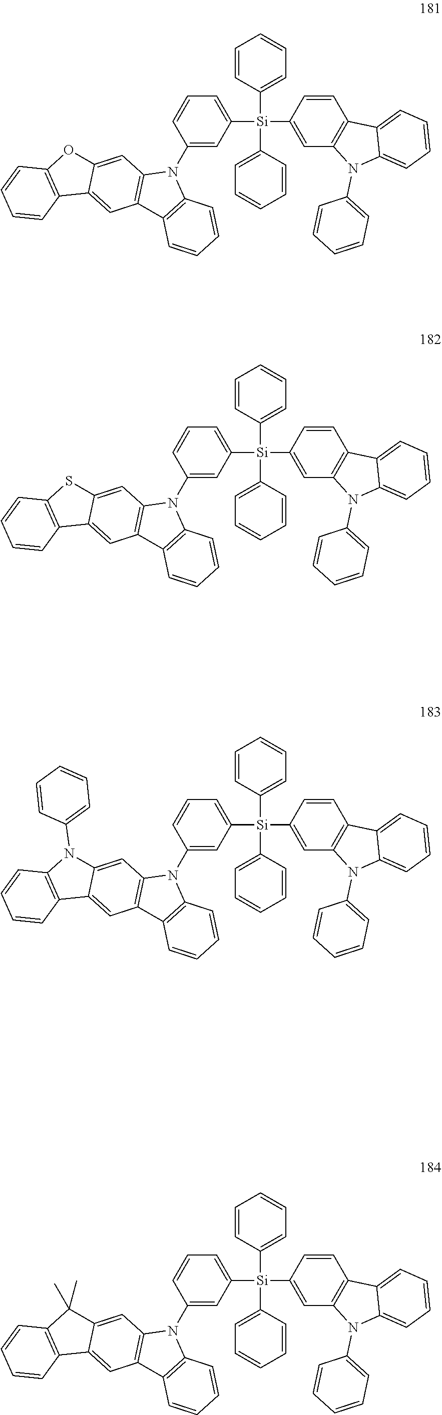 [SMILES: c1ccc(-n2c3ccccc3c3ccc([Si](c4ccccc4)(c4ccccc4)c4cccc(-n5c6ccccc6c6cc7c(cc65)oc5ccccc57)c4)cc32)cc1 ; c1ccc(-n2c3ccccc3c3ccc([Si](c4ccccc4)(c4ccccc4)c4cccc(-n5c6ccccc6c6cc7c(cc65)sc5ccccc57)c4)cc32)cc1 ; c1ccc(-n2c3ccccc3c3ccc([Si](c4ccccc4)(c4ccccc4)c4cccc(-n5c6ccccc6c6cc7c8ccccc8n(-c8ccccc8)c7cc65)c4)cc32)cc1 ; CC1(C)c2ccccc2-c2cc3c4ccccc4n(-c4cccc([Si](c5ccccc5)(c5ccccc5)c5ccc6c7ccccc7n(-c7ccccc7)c6c5)c4)c3cc21]

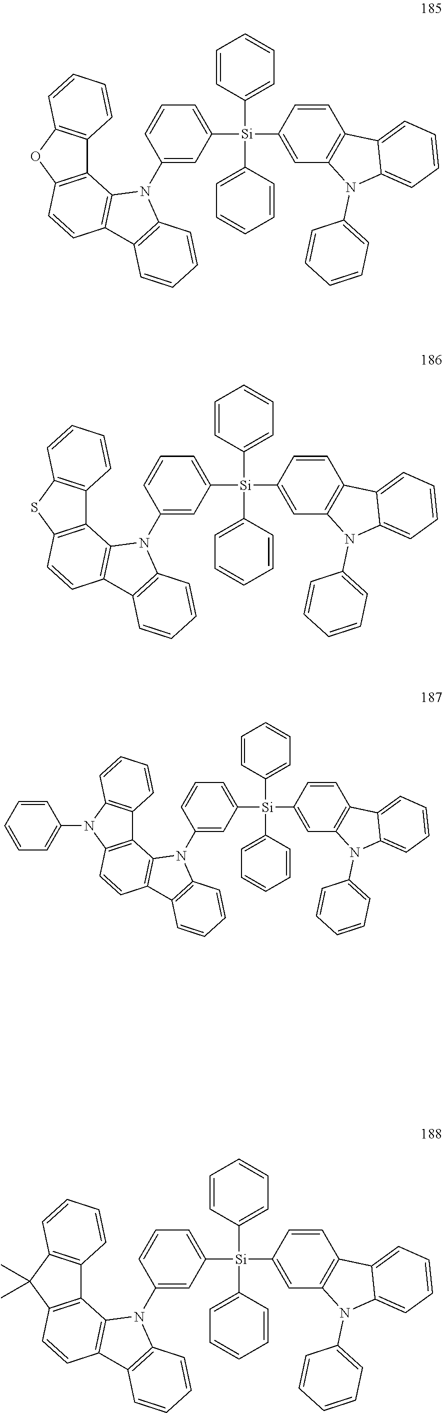 [SMILES: c1ccc(-n2c3ccccc3c3ccc([Si](c4ccccc4)(c4ccccc4)c4cccc(-n5c6ccccc6c6ccc7oc8ccccc8c7c65)c4)cc32)cc1 ; c1ccc(-n2c3ccccc3c3ccc([Si](c4ccccc4)(c4ccccc4)c4cccc(-n5c6ccccc6c6ccc7sc8ccccc8c7c65)c4)cc32)cc1 ; c1ccc(-n2c3ccccc3c3ccc([Si](c4ccccc4)(c4ccccc4)c4cccc(-n5c6ccccc6c6ccc7c(c8ccccc8n7-c7ccccc7)c65)c4)cc32)cc1 ; CC1(C)c2ccccc2-c2c1ccc1c3ccccc3n(-c3cccc([Si](c4ccccc4)(c4ccccc4)c4ccc5c6ccccc6n(-c6ccccc6)c5c4)c3)c21]

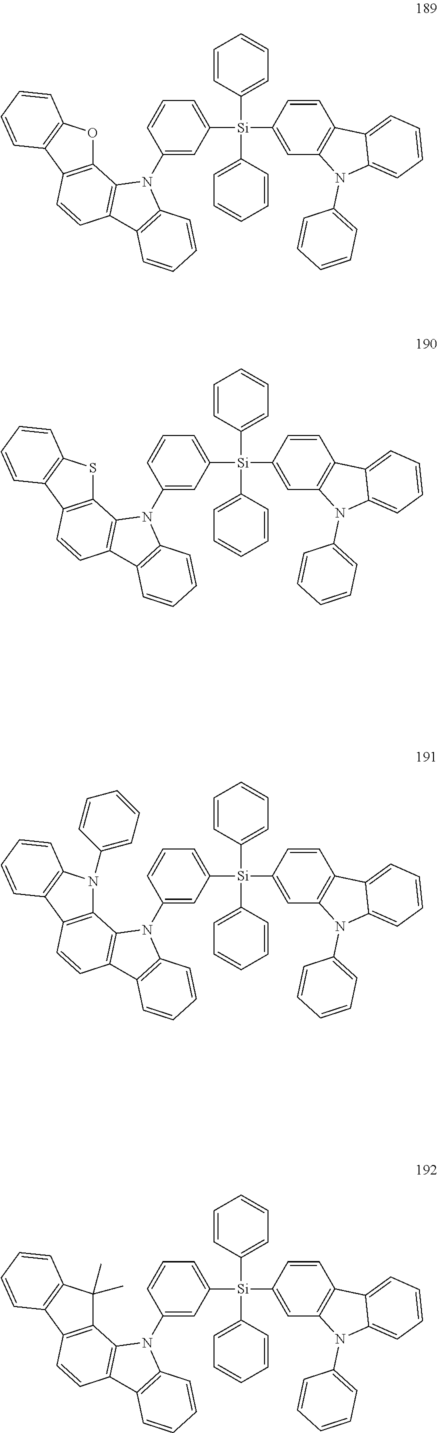 [SMILES: c1ccc(-n2c3ccccc3c3ccc([Si](c4ccccc4)(c4ccccc4)c4cccc(-n5c6ccccc6c6ccc7c8ccccc8oc7c65)c4)cc32)cc1 ; c1ccc(-n2c3ccccc3c3ccc([Si](c4ccccc4)(c4ccccc4)c4cccc(-n5c6ccccc6c6ccc7c8ccccc8sc7c65)c4)cc32)cc1 ; c1ccc(-n2c3ccccc3c3ccc([Si](c4ccccc4)(c4ccccc4)c4cccc(-n5c6ccccc6c6ccc7c8ccccc8n(-c8ccccc8)c7c65)c4)cc32)cc1 ; CC1(C)c2ccccc2-c2ccc3c4ccccc4n(-c4cccc([Si](c5ccccc5)(c5ccccc5)c5ccc6c7ccccc7n(-c7ccccc7)c6c5)c4)c3c21]

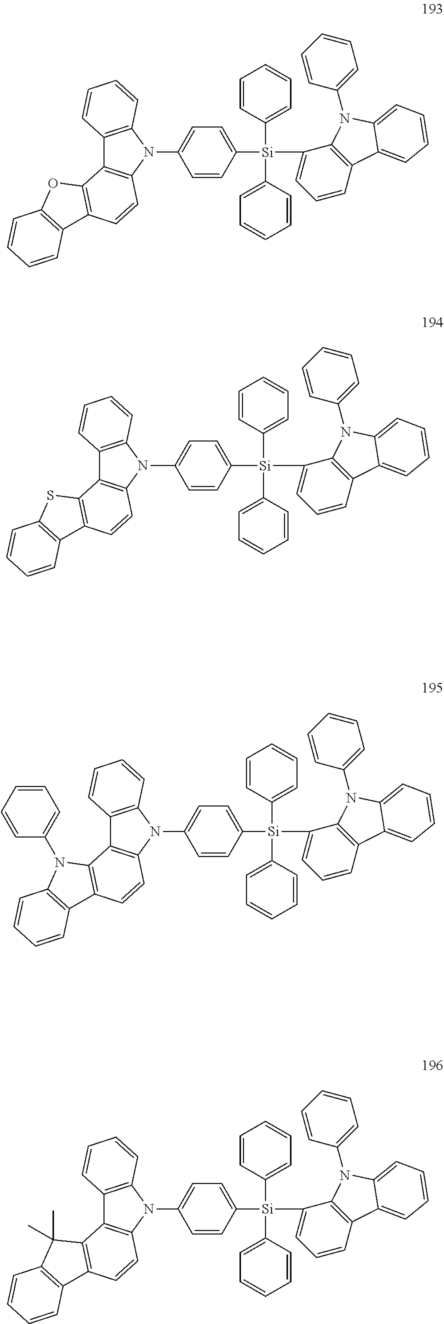 [SMILES: c1ccc(-n2c3ccccc3c3cccc([Si](c4ccccc4)(c4ccccc4)c4ccc(-n5c6ccccc6c6c7oc8ccccc8c7ccc65)cc4)c32)cc1 ; c1ccc(-n2c3ccccc3c3cccc([Si](c4ccccc4)(c4ccccc4)c4ccc(-n5c6ccccc6c6c7sc8ccccc8c7ccc65)cc4)c32)cc1 ; c1ccc(-n2c3ccccc3c3cccc([Si](c4ccccc4)(c4ccccc4)c4ccc(-n5c6ccccc6c6c5ccc5c7ccccc7n(-c7ccccc7)c56)cc4)c32)cc1 ; CC1(C)c2ccccc2-c2ccc3c(c21)c1ccccc1n3-c1ccc([Si](c2ccccc2)(c2ccccc2)c2cccc3c4ccccc4n(-c4ccccc4)c23)cc1]

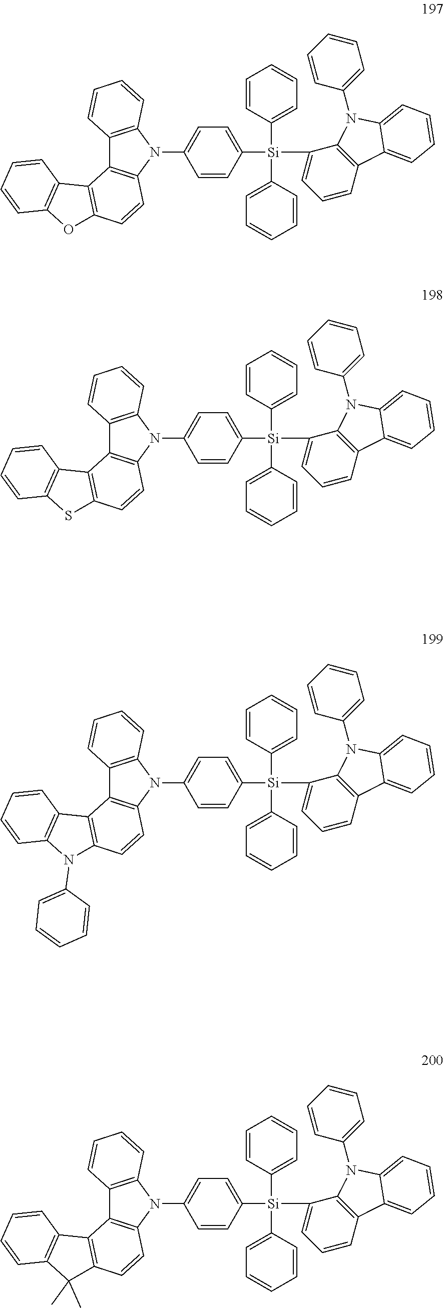 [SMILES: c1ccc(-n2c3ccccc3c3cccc([Si](c4ccccc4)(c4ccccc4)c4ccc(-n5c6ccccc6c6c7c(ccc65)oc5ccccc57)cc4)c32)cc1 ; c1ccc(-n2c3ccccc3c3cccc([Si](c4ccccc4)(c4ccccc4)c4ccc(-n5c6ccccc6c6c7c(ccc65)sc5ccccc57)cc4)c32)cc1 ; c1ccc(-n2c3ccccc3c3c4c5ccccc5n(-c5ccc([Si](c6ccccc6)(c6ccccc6)c6cccc7c8ccccc8n(-c8ccccc8)c67)cc5)c4ccc32)cc1 ; CC1(C)c2ccccc2-c2c1ccc1c2c2ccccc2n1-c1ccc([Si](c2ccccc2)(c2ccccc2)c2cccc3c4ccccc4n(-c4ccccc4)c23)cc1]

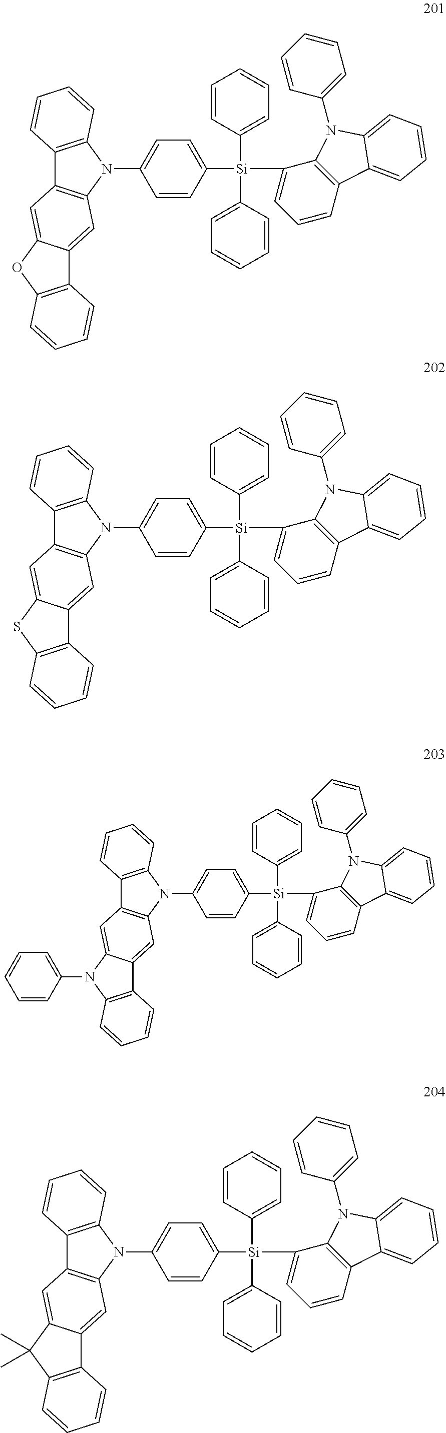 [SMILES: c1ccc(-n2c3ccccc3c3cccc([Si](c4ccccc4)(c4ccccc4)c4ccc(-n5c6ccccc6c6cc7oc8ccccc8c7cc65)cc4)c32)cc1 ; c1ccc(-n2c3ccccc3c3cccc([Si](c4ccccc4)(c4ccccc4)c4ccc(-n5c6ccccc6c6cc7sc8ccccc8c7cc65)cc4)c32)cc1 ; c1ccc(-n2c3ccccc3c3cc4c(cc32)c2ccccc2n4-c2ccc([Si](c3ccccc3)(c3ccccc3)c3cccc4c5ccccc5n(-c5ccccc5)c34)cc2)cc1 ; CC1(C)c2ccccc2-c2cc3c(cc21)c1ccccc1n3-c1ccc([Si](c2ccccc2)(c2ccccc2)c2cccc3c4ccccc4n(-c4ccccc4)c23)cc1]

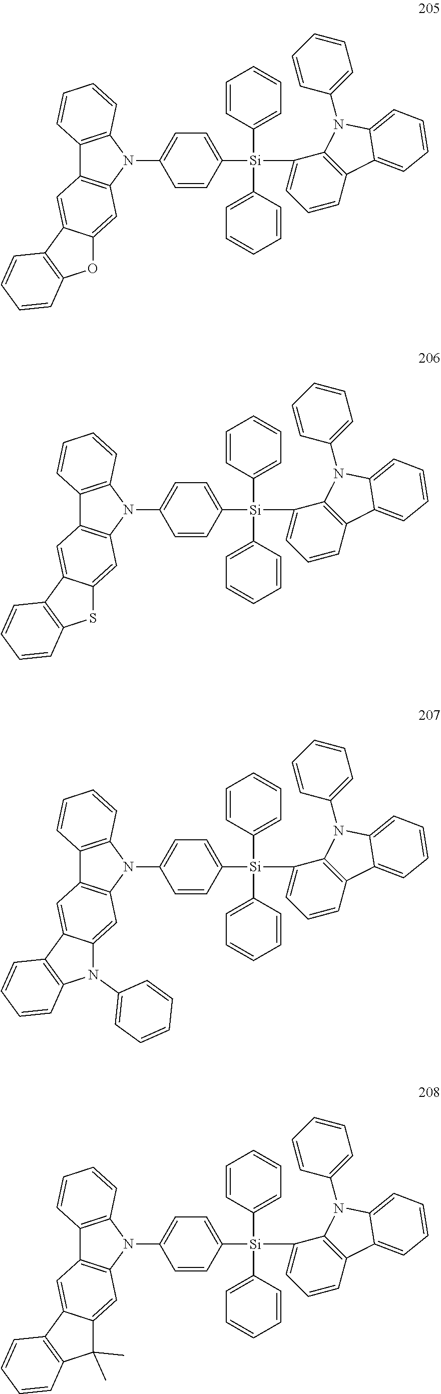 [SMILES: c1ccc(-n2c3ccccc3c3cccc([Si](c4ccccc4)(c4ccccc4)c4ccc(-n5c6ccccc6c6cc7c(cc65)oc5ccccc57)cc4)c32)cc1 ; c1ccc(-n2c3ccccc3c3cccc([Si](c4ccccc4)(c4ccccc4)c4ccc(-n5c6ccccc6c6cc7c(cc65)sc5ccccc57)cc4)c32)cc1 ; c1ccc(-n2c3ccccc3c3cc4c5ccccc5n(-c5ccc([Si](c6ccccc6)(c6ccccc6)c6cccc7c8ccccc8n(-c8ccccc8)c67)cc5)c4cc32)cc1 ; CC1(C)c2ccccc2-c2cc3c4ccccc4n(-c4ccc([Si](c5ccccc5)(c5ccccc5)c5cccc6c7ccccc7n(-c7ccccc7)c56)cc4)c3cc21]

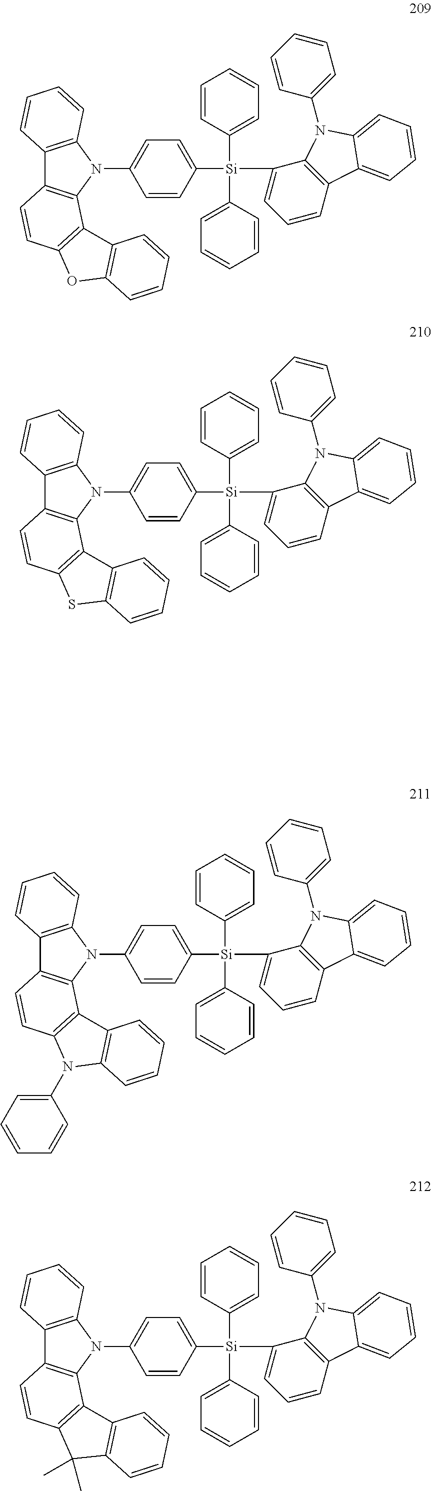 [SMILES: c1ccc(-n2c3ccccc3c3cccc([Si](c4ccccc4)(c4ccccc4)c4ccc(-n5c6ccccc6c6ccc7oc8ccccc8c7c65)cc4)c32)cc1 ; c1ccc(-n2c3ccccc3c3cccc([Si](c4ccccc4)(c4ccccc4)c4ccc(-n5c6ccccc6c6ccc7sc8ccccc8c7c65)cc4)c32)cc1 ; c1ccc(-n2c3ccccc3c3c2ccc2c4ccccc4n(-c4ccc([Si](c5ccccc5)(c5ccccc5)c5cccc6c7ccccc7n(-c7ccccc7)c56)cc4)c23)cc1 ; CC1(C)c2ccccc2-c2c1ccc1c3ccccc3n(-c3ccc([Si](c4ccccc4)(c4ccccc4)c4cccc5c6ccccc6n(-c6ccccc6)c45)cc3)c21]

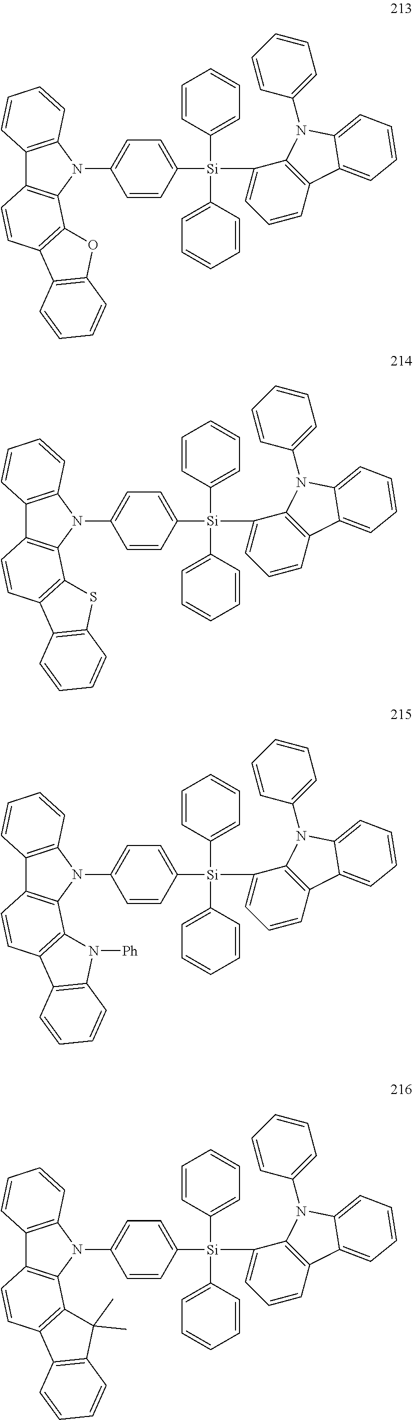 [SMILES: c1ccc(-n2c3ccccc3c3cccc([Si](c4ccccc4)(c4ccccc4)c4ccc(-n5c6ccccc6c6ccc7c8ccccc8oc7c65)cc4)c32)cc1 ; c1ccc(-n2c3ccccc3c3cccc([Si](c4ccccc4)(c4ccccc4)c4ccc(-n5c6ccccc6c6ccc7c8ccccc8sc7c65)cc4)c32)cc1 ; c1ccc(-n2c3ccccc3c3cccc([Si](c4ccccc4)(c4ccccc4)c4ccc(-n5c6ccccc6c6ccc7c8ccccc8n(-c8ccccc8)c7c65)cc4)c32)cc1 ; CC1(C)c2ccccc2-c2ccc3c4ccccc4n(-c4ccc([Si](c5ccccc5)(c5ccccc5)c5cccc6c7ccccc7n(-c7ccccc7)c56)cc4)c3c21]

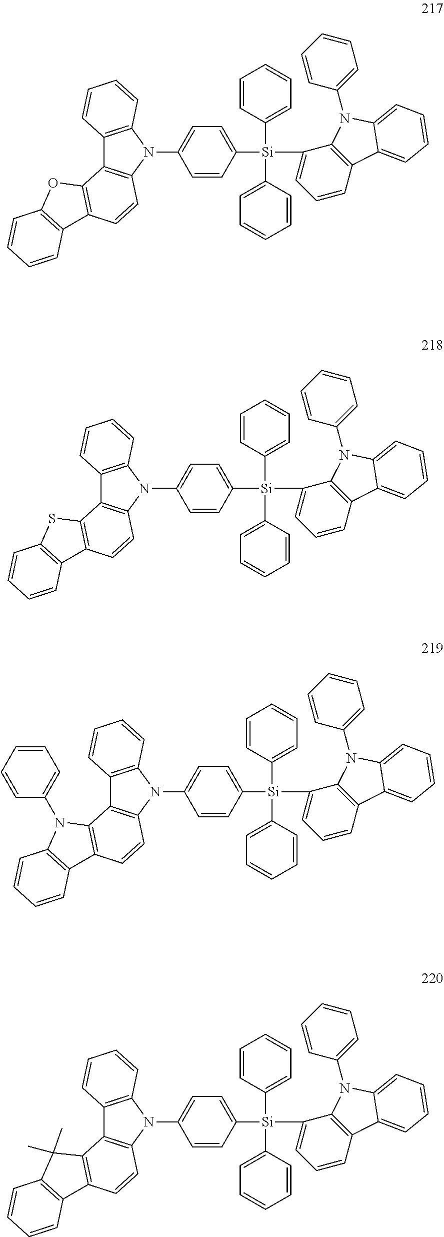 [SMILES: c1ccc(-n2c3ccccc3c3cccc([Si](c4ccccc4)(c4ccccc4)c4ccc(-n5c6ccccc6c6c7oc8ccccc8c7ccc65)cc4)c32)cc1 ; c1ccc(-n2c3ccccc3c3cccc([Si](c4ccccc4)(c4ccccc4)c4ccc(-n5c6ccccc6c6c7sc8ccccc8c7ccc65)cc4)c32)cc1 ; c1ccc(-n2c3ccccc3c3cccc([Si](c4ccccc4)(c4ccccc4)c4ccc(-n5c6ccccc6c6c5ccc5c7ccccc7n(-c7ccccc7)c56)cc4)c32)cc1 ; CC1(C)c2ccccc2-c2ccc3c(c21)c1ccccc1n3-c1ccc([Si](c2ccccc2)(c2ccccc2)c2cccc3c4ccccc4n(-c4ccccc4)c23)cc1]

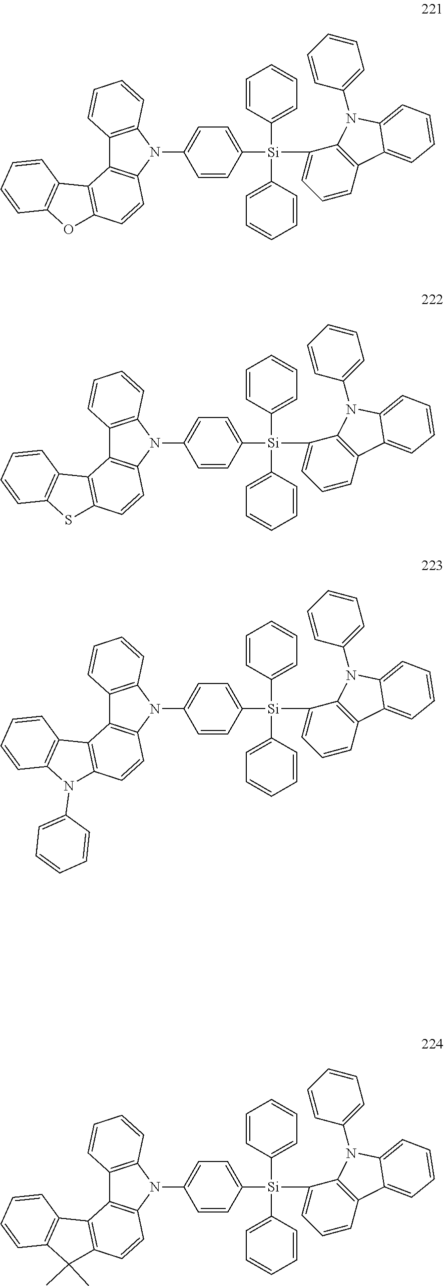 [SMILES: c1ccc(-n2c3ccccc3c3cccc([Si](c4ccccc4)(c4ccccc4)c4ccc(-n5c6ccccc6c6c7c(ccc65)oc5ccccc57)cc4)c32)cc1 ; c1ccc(-n2c3ccccc3c3cccc([Si](c4ccccc4)(c4ccccc4)c4ccc(-n5c6ccccc6c6c7c(ccc65)sc5ccccc57)cc4)c32)cc1 ; c1ccc(-n2c3ccccc3c3c4c5ccccc5n(-c5ccc([Si](c6ccccc6)(c6ccccc6)c6cccc7c8ccccc8n(-c8ccccc8)c67)cc5)c4ccc32)cc1 ; CC1(C)c2ccccc2-c2c1ccc1c2c2ccccc2n1-c1ccc([Si](c2ccccc2)(c2ccccc2)c2cccc3c4ccccc4n(-c4ccccc4)c23)cc1]

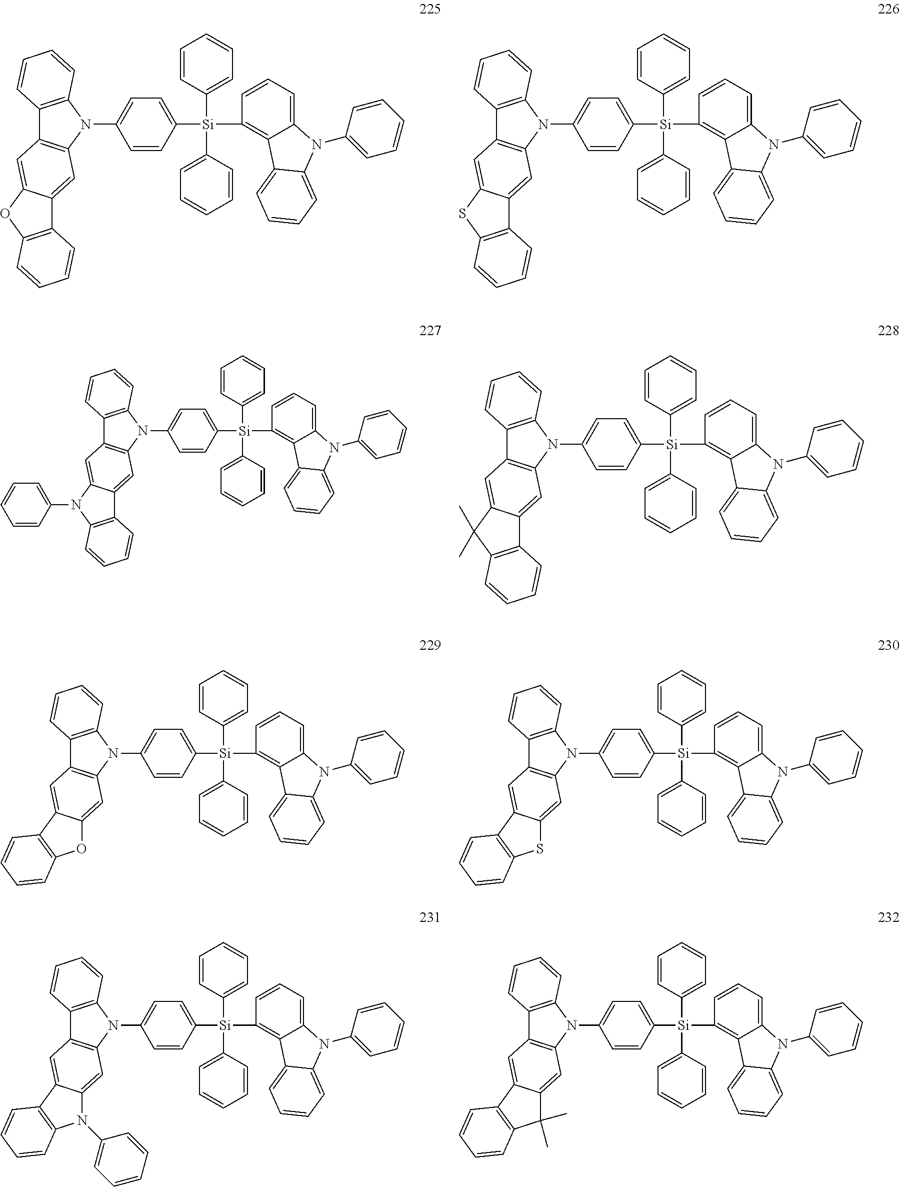

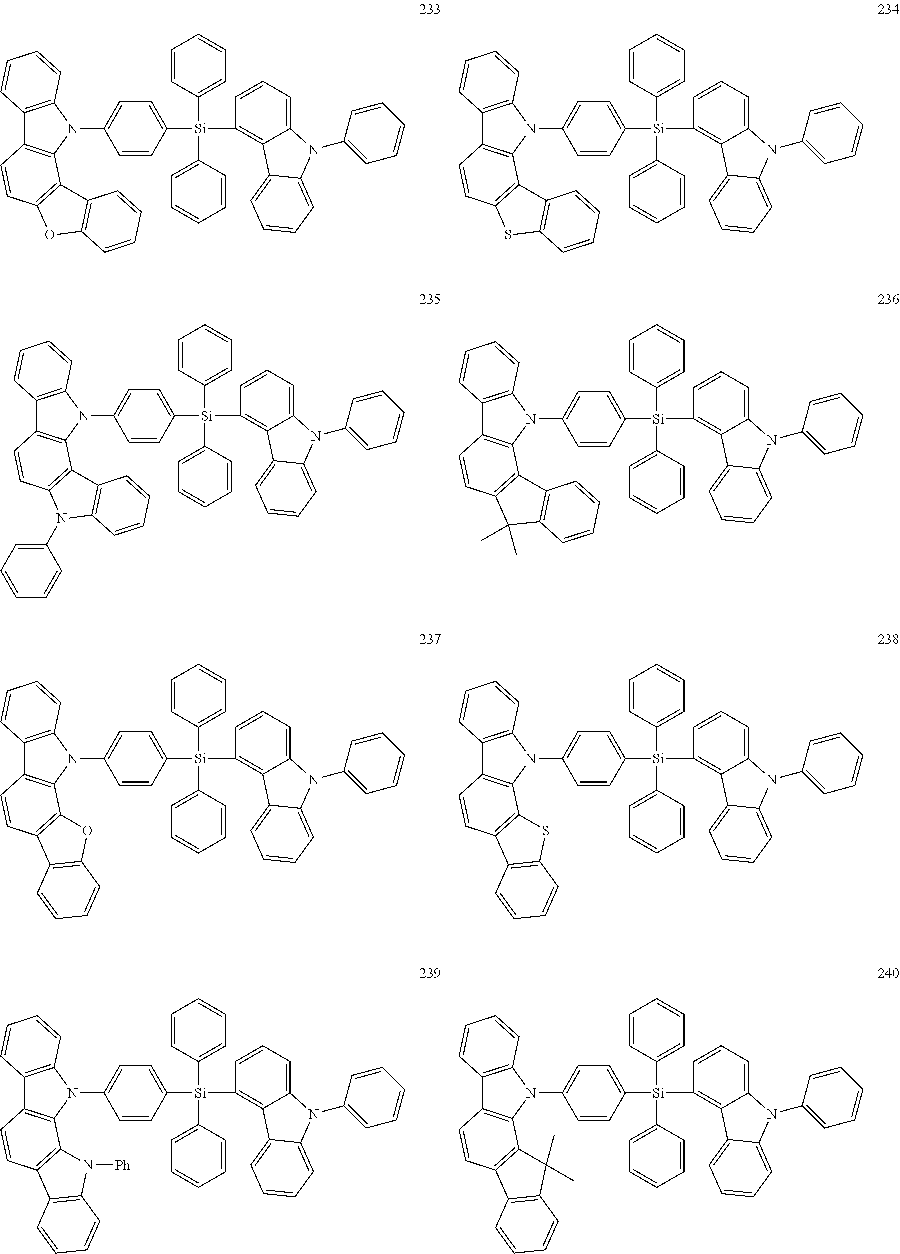

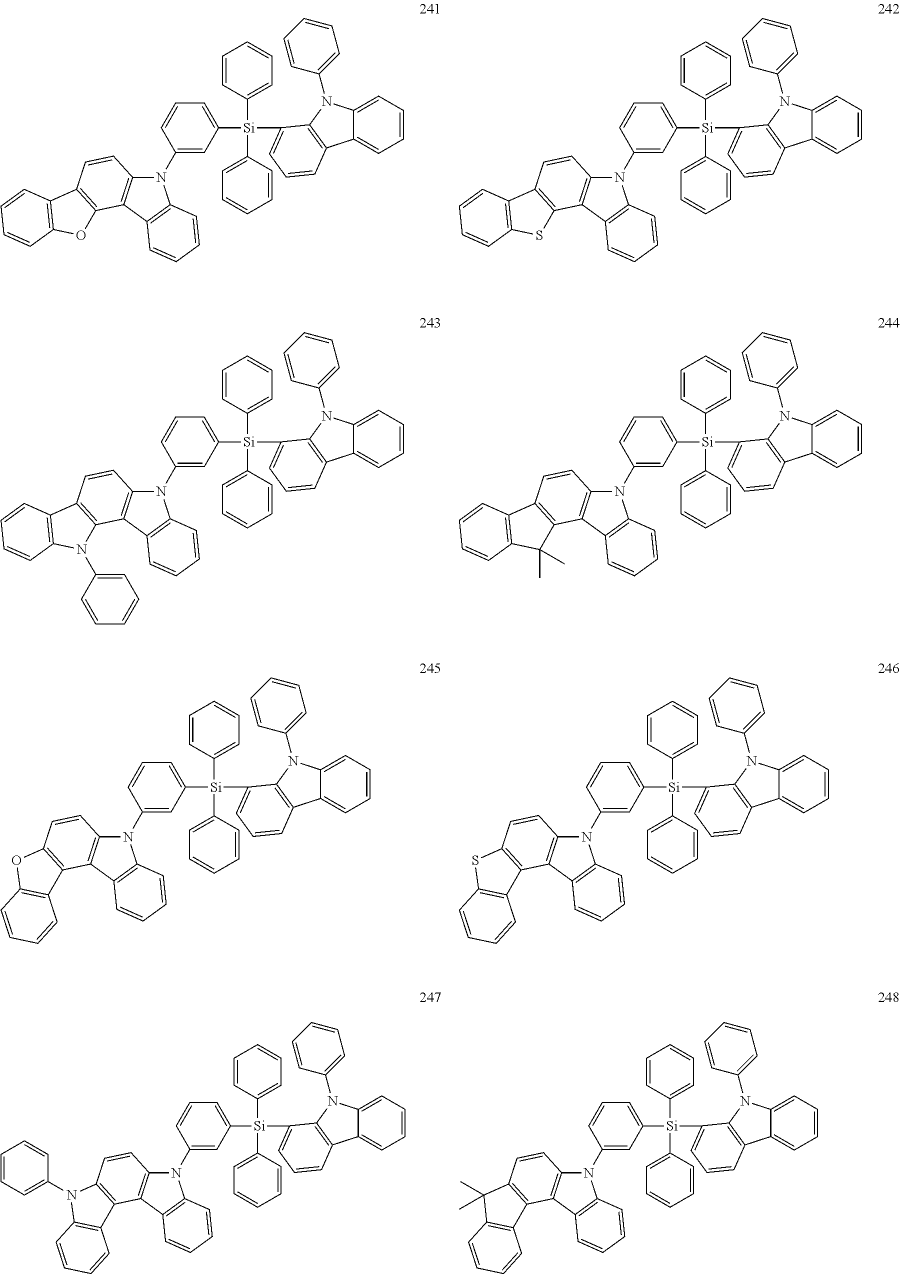

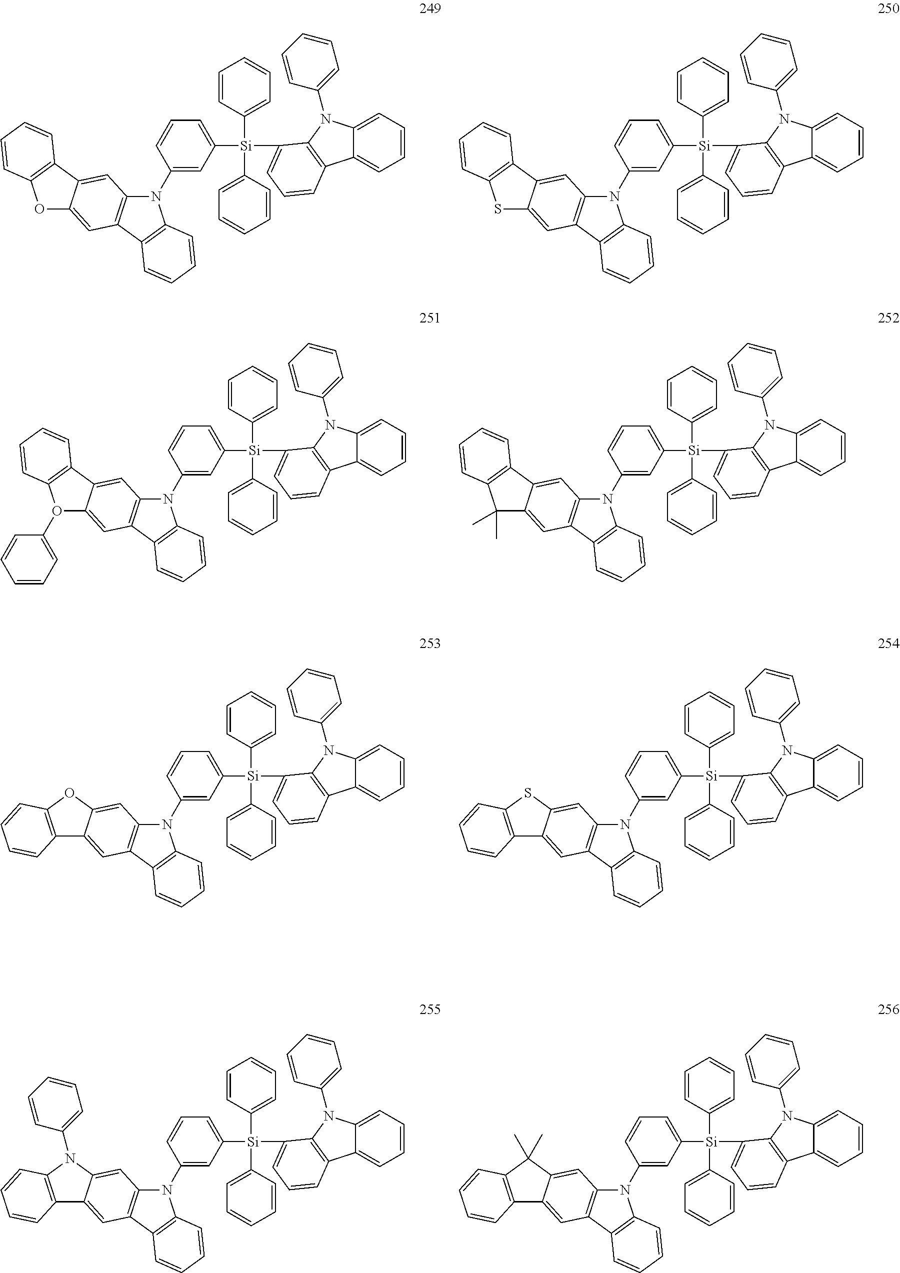

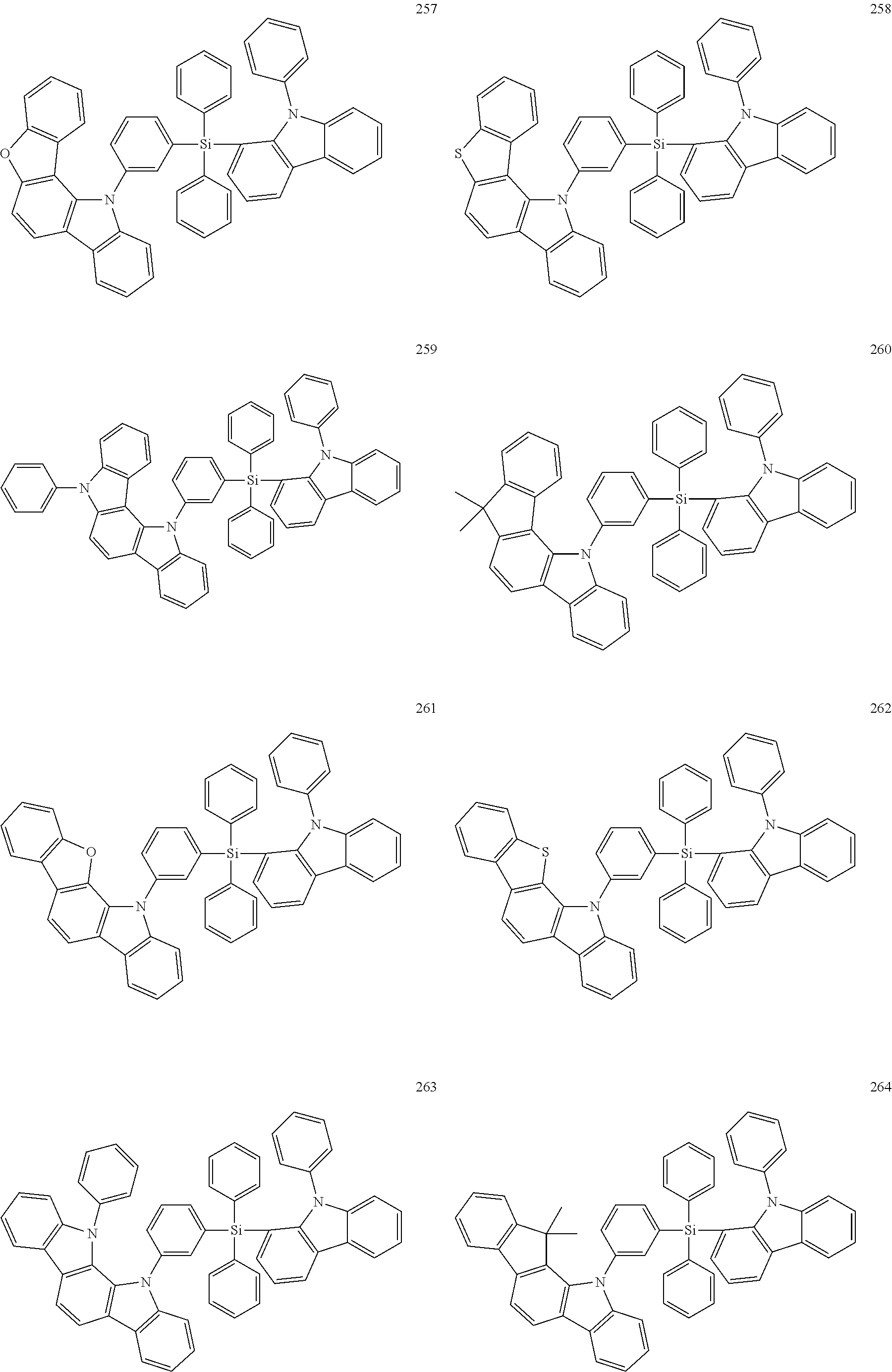

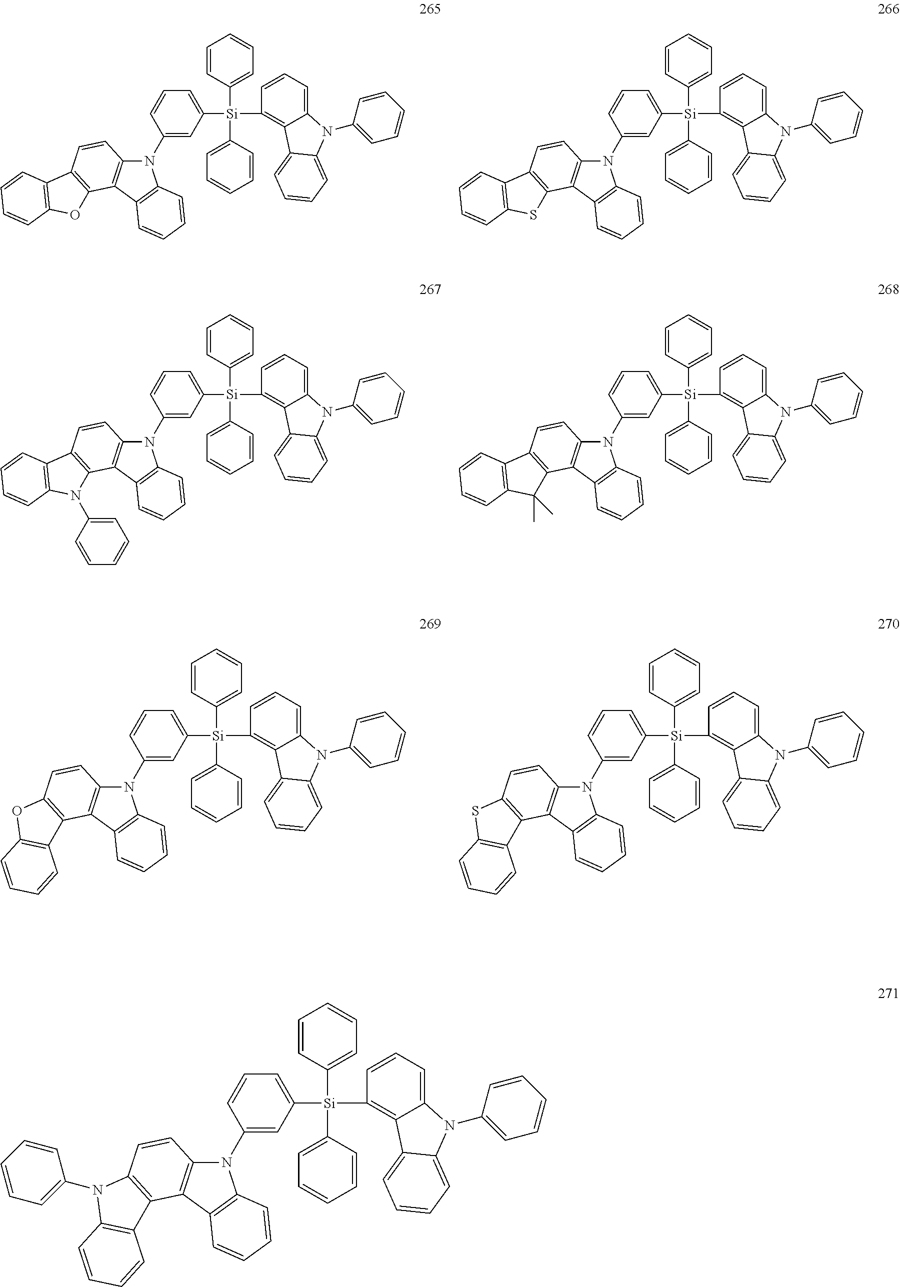

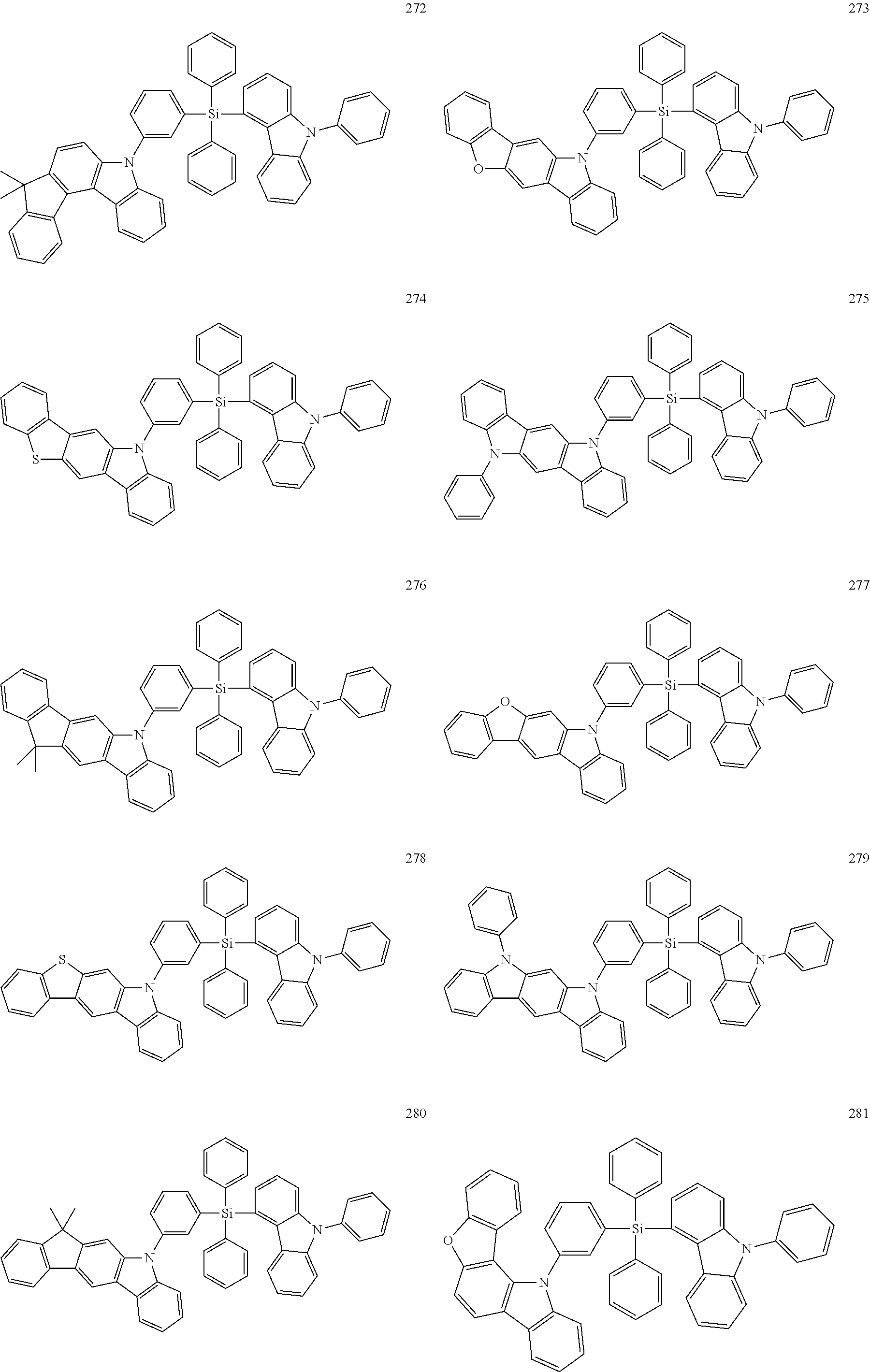

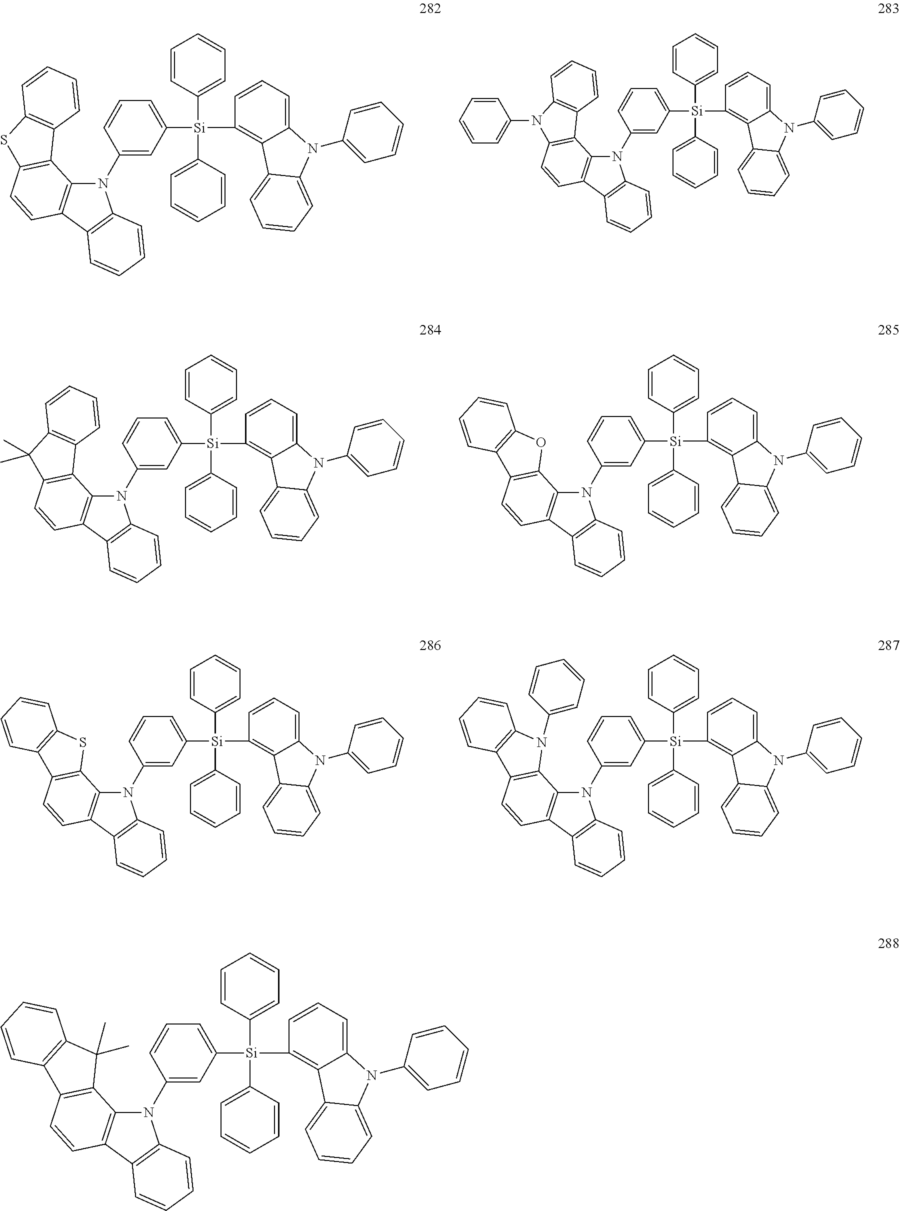

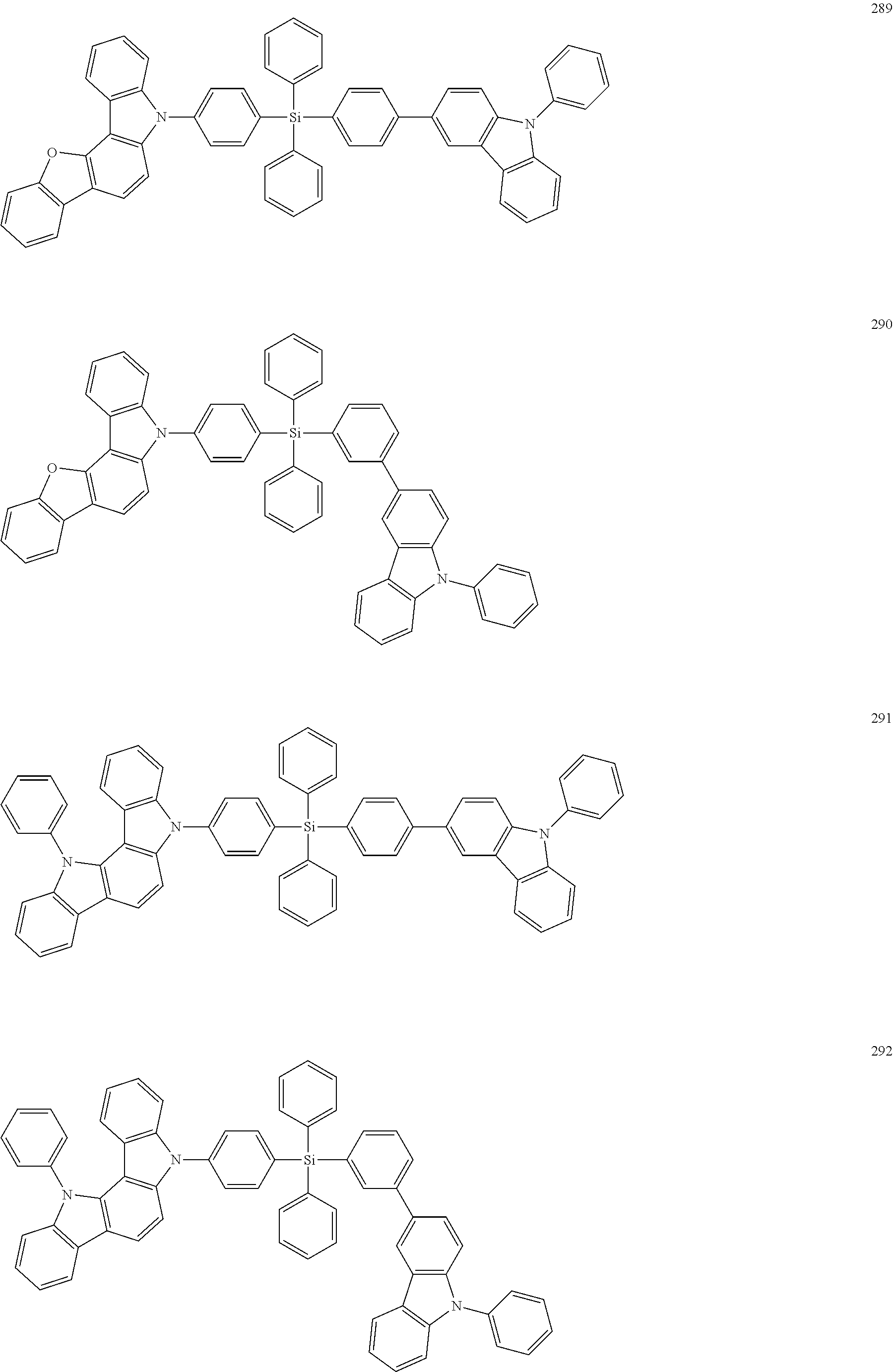 [SMILES: c1ccc(-n2c3ccccc3c3cc(-c4ccc([Si](c5ccccc5)(c5ccccc5)c5ccc(-n6c7ccccc7c7c8oc9ccccc9c8ccc76)cc5)cc4)ccc32)cc1 ; c1ccc(-n2c3ccccc3c3cc(-c4cccc([Si](c5ccccc5)(c5ccccc5)c5ccc(-n6c7ccccc7c7c8oc9ccccc9c8ccc76)cc5)c4)ccc32)cc1 ; c1ccc(-n2c3ccccc3c3cc(-c4ccc([Si](c5ccccc5)(c5ccccc5)c5ccc(-n6c7ccccc7c7c6ccc6c8ccccc8n(-c8ccccc8)c67)cc5)cc4)ccc32)cc1 ; c1ccc(-n2c3ccccc3c3cc(-c4cccc([Si](c5ccccc5)(c5ccccc5)c5ccc(-n6c7ccccc7c7c6ccc6c8ccccc8n(-c8ccccc8)c67)cc5)c4)ccc32)cc1]

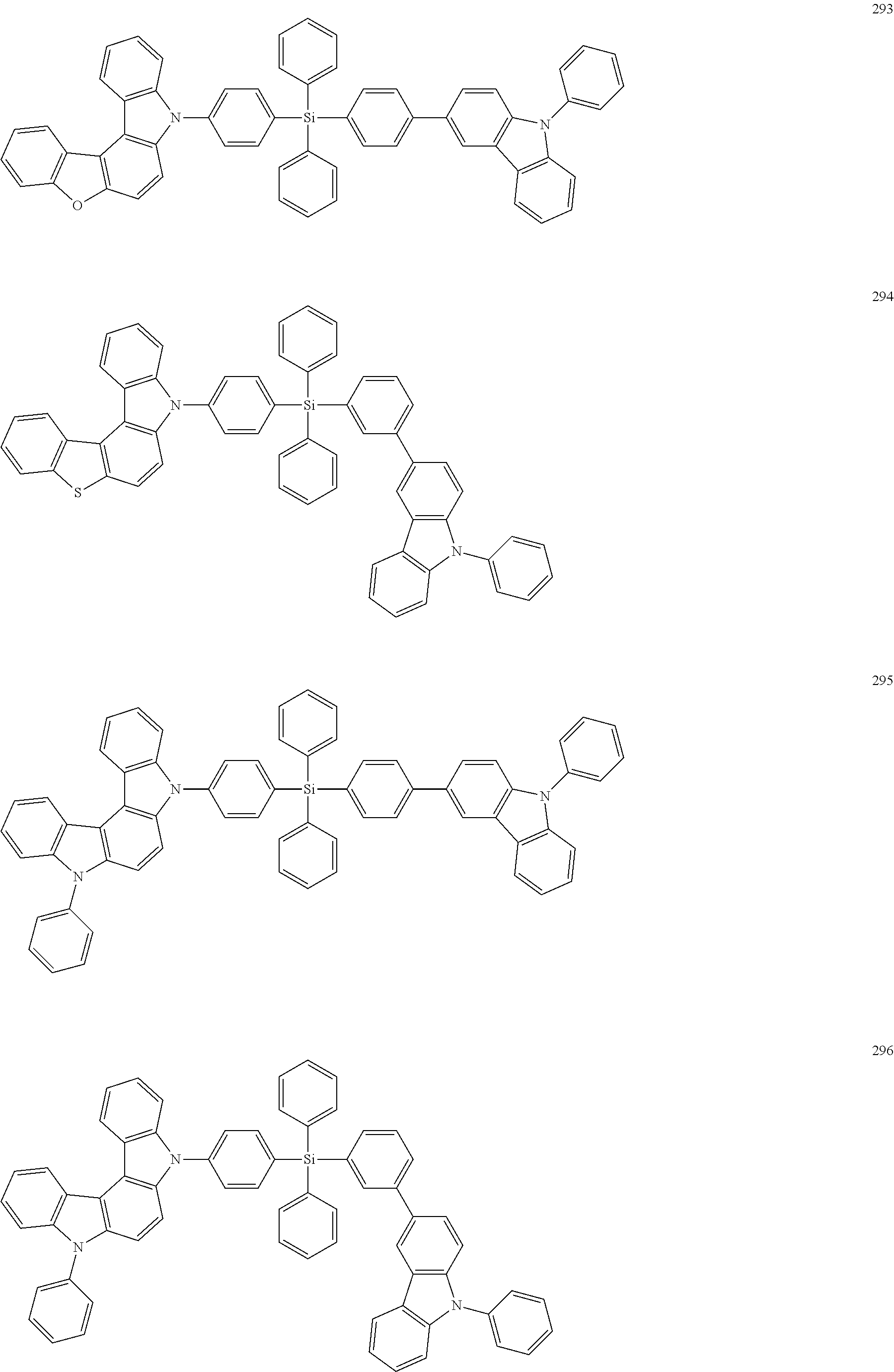 [SMILES: c1ccc(-n2c3ccccc3c3cc(-c4ccc([Si](c5ccccc5)(c5ccccc5)c5ccc(-n6c7ccccc7c7c8c(ccc76)oc6ccccc68)cc5)cc4)ccc32)cc1 ; c1ccc(-n2c3ccccc3c3cc(-c4cccc([Si](c5ccccc5)(c5ccccc5)c5ccc(-n6c7ccccc7c7c8c(ccc76)sc6ccccc68)cc5)c4)ccc32)cc1 ; c1ccc(-n2c3ccccc3c3cc(-c4ccc([Si](c5ccccc5)(c5ccccc5)c5ccc(-n6c7ccccc7c7c8c9ccccc9n(-c9ccccc9)c8ccc76)cc5)cc4)ccc32)cc1 ; c1ccc(-n2c3ccccc3c3cc(-c4cccc([Si](c5ccccc5)(c5ccccc5)c5ccc(-n6c7ccccc7c7c8c9ccccc9n(-c9ccccc9)c8ccc76)cc5)c4)ccc32)cc1]

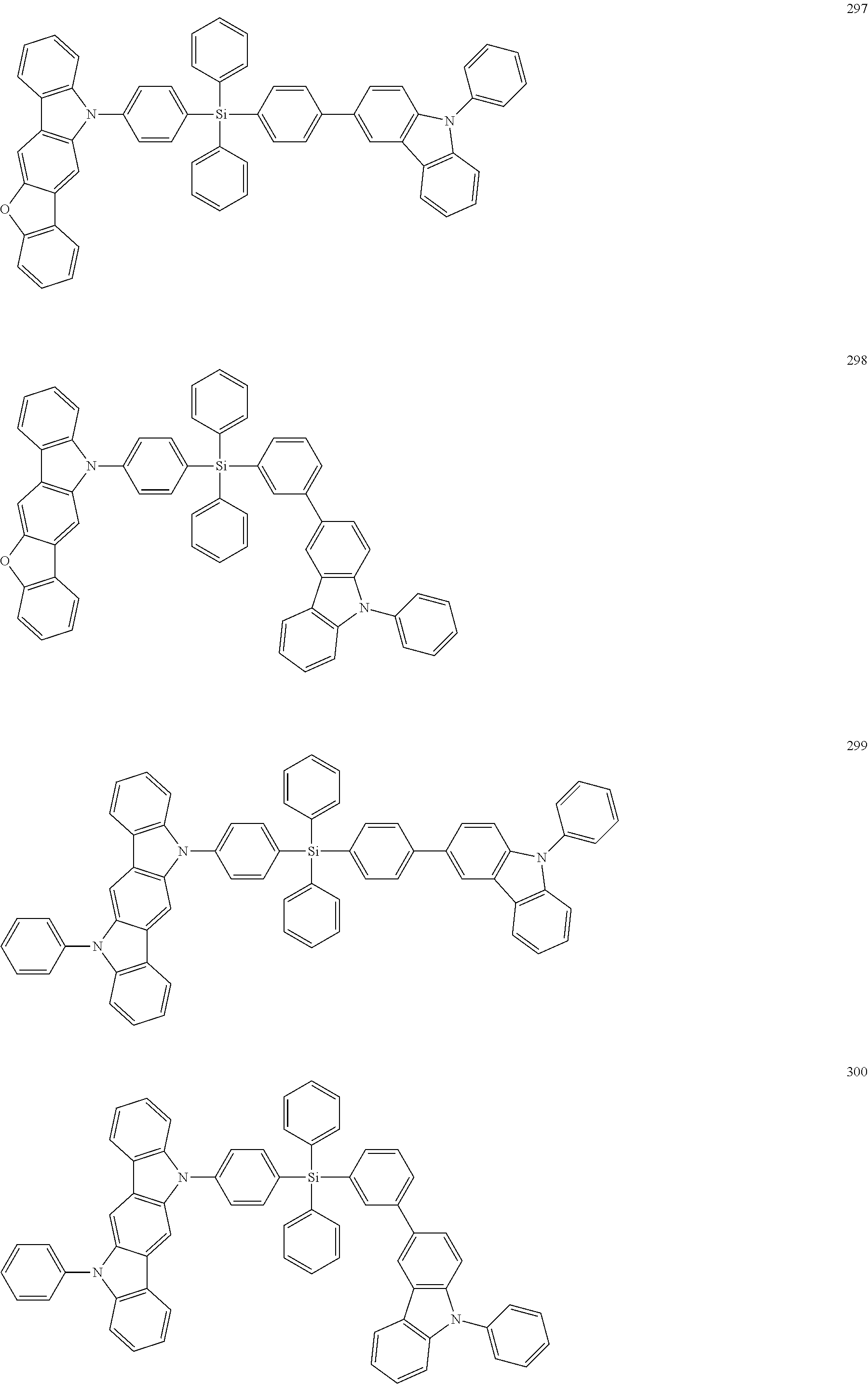 [SMILES: c1ccc(-n2c3ccccc3c3cc(-c4ccc([Si](c5ccccc5)(c5ccccc5)c5ccc(-n6c7ccccc7c7cc8oc9ccccc9c8cc76)cc5)cc4)ccc32)cc1 ; c1ccc(-n2c3ccccc3c3cc(-c4cccc([Si](c5ccccc5)(c5ccccc5)c5ccc(-n6c7ccccc7c7cc8oc9ccccc9c8cc76)cc5)c4)ccc32)cc1 ; c1ccc(-n2c3ccccc3c3cc(-c4ccc([Si](c5ccccc5)(c5ccccc5)c5ccc(-n6c7ccccc7c7cc8c(cc76)c6ccccc6n8-c6ccccc6)cc5)cc4)ccc32)cc1 ; c1ccc(-n2c3ccccc3c3cc(-c4cccc([Si](c5ccccc5)(c5ccccc5)c5ccc(-n6c7ccccc7c7cc8c(cc76)c6ccccc6n8-c6ccccc6)cc5)c4)ccc32)cc1]

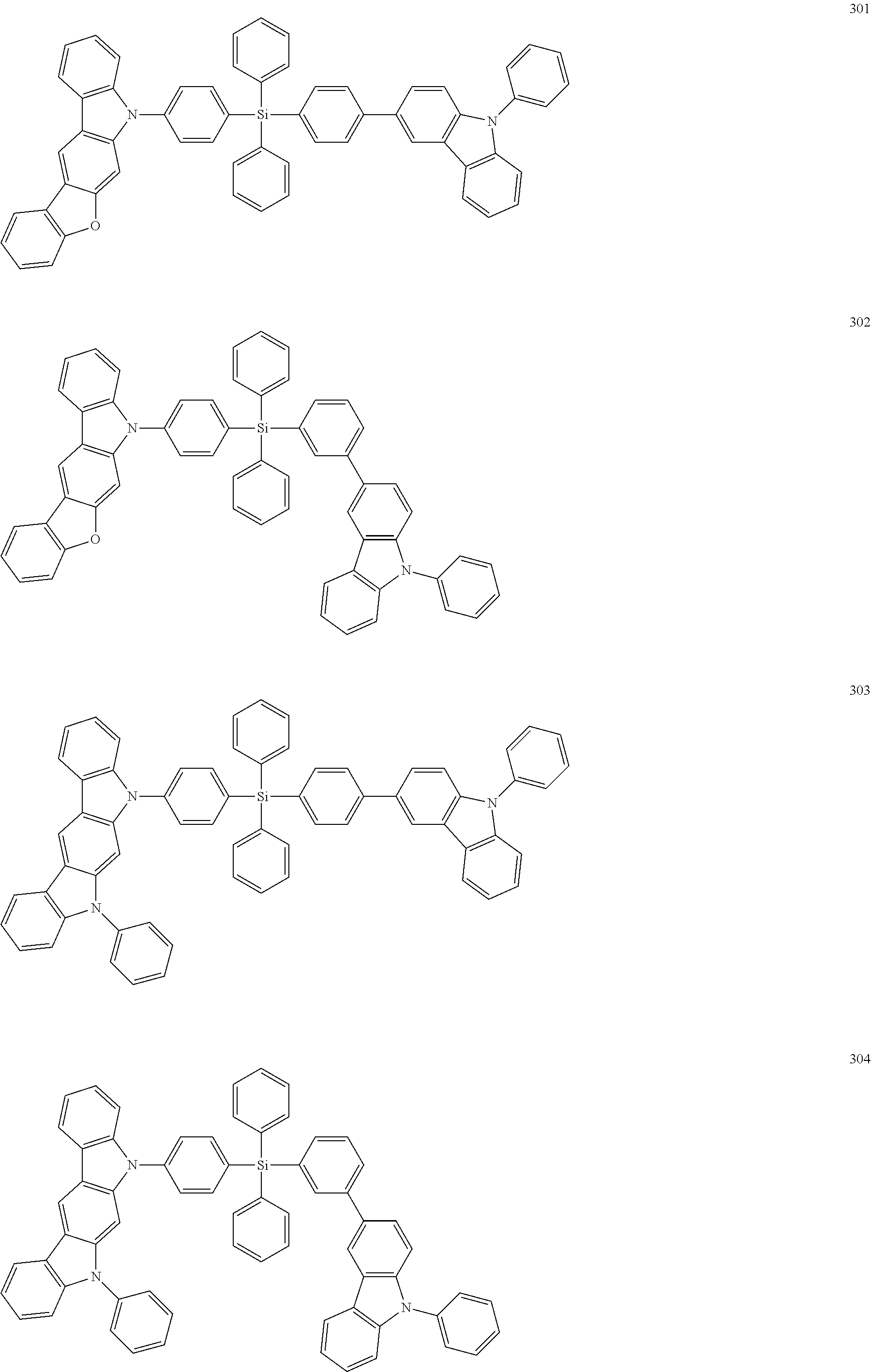 [SMILES: c1ccc(-n2c3ccccc3c3cc(-c4ccc([Si](c5ccccc5)(c5ccccc5)c5ccc(-n6c7ccccc7c7cc8c(cc76)oc6ccccc68)cc5)cc4)ccc32)cc1 ; c1ccc(-n2c3ccccc3c3cc(-c4cccc([Si](c5ccccc5)(c5ccccc5)c5ccc(-n6c7ccccc7c7cc8c(cc76)oc6ccccc68)cc5)c4)ccc32)cc1 ; c1ccc(-n2c3ccccc3c3cc(-c4ccc([Si](c5ccccc5)(c5ccccc5)c5ccc(-n6c7ccccc7c7cc8c9ccccc9n(-c9ccccc9)c8cc76)cc5)cc4)ccc32)cc1 ; c1ccc(-n2c3ccccc3c3cc(-c4cccc([Si](c5ccccc5)(c5ccccc5)c5ccc(-n6c7ccccc7c7cc8c9ccccc9n(-c9ccccc9)c8cc76)cc5)c4)ccc32)cc1]

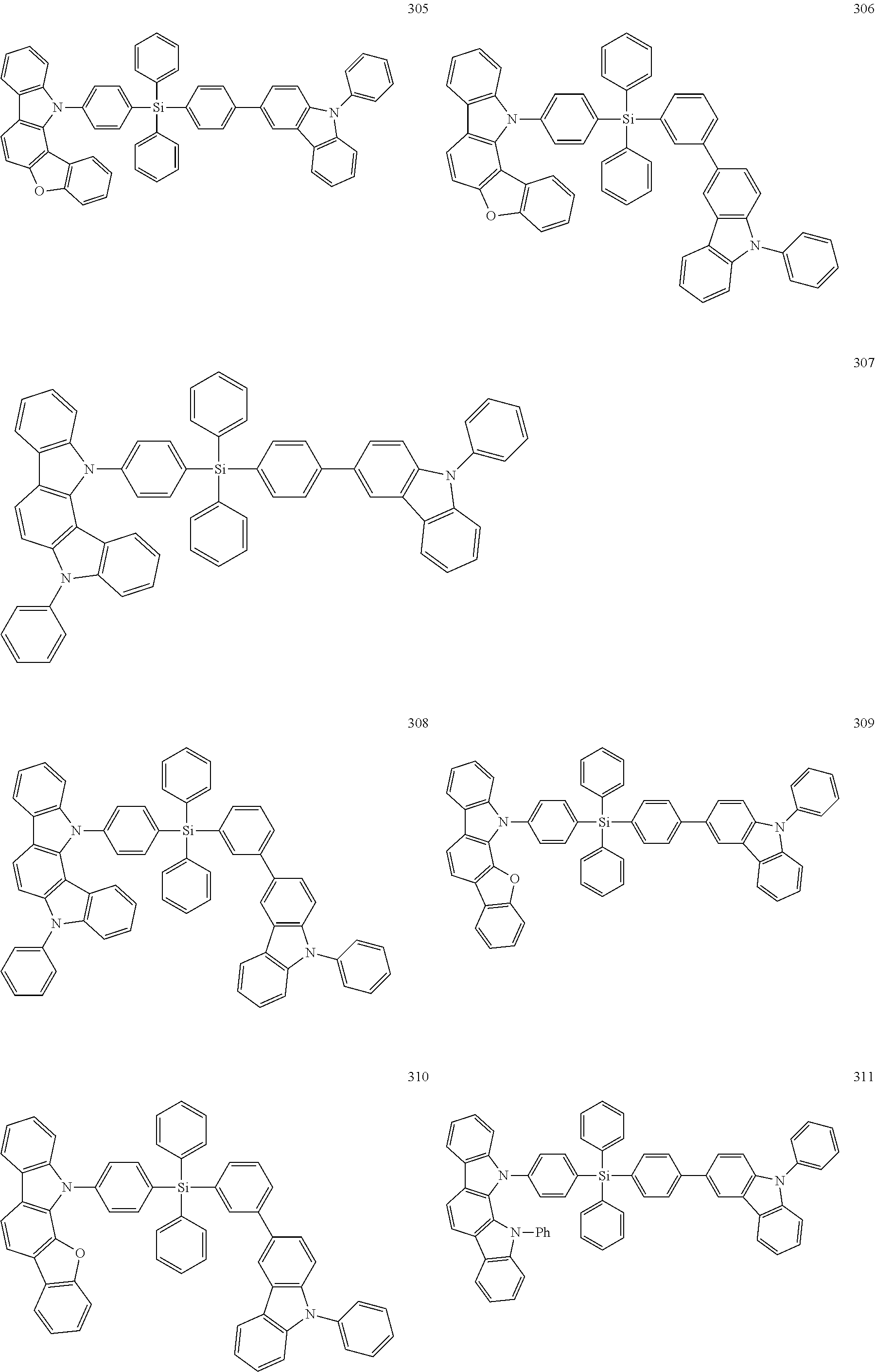

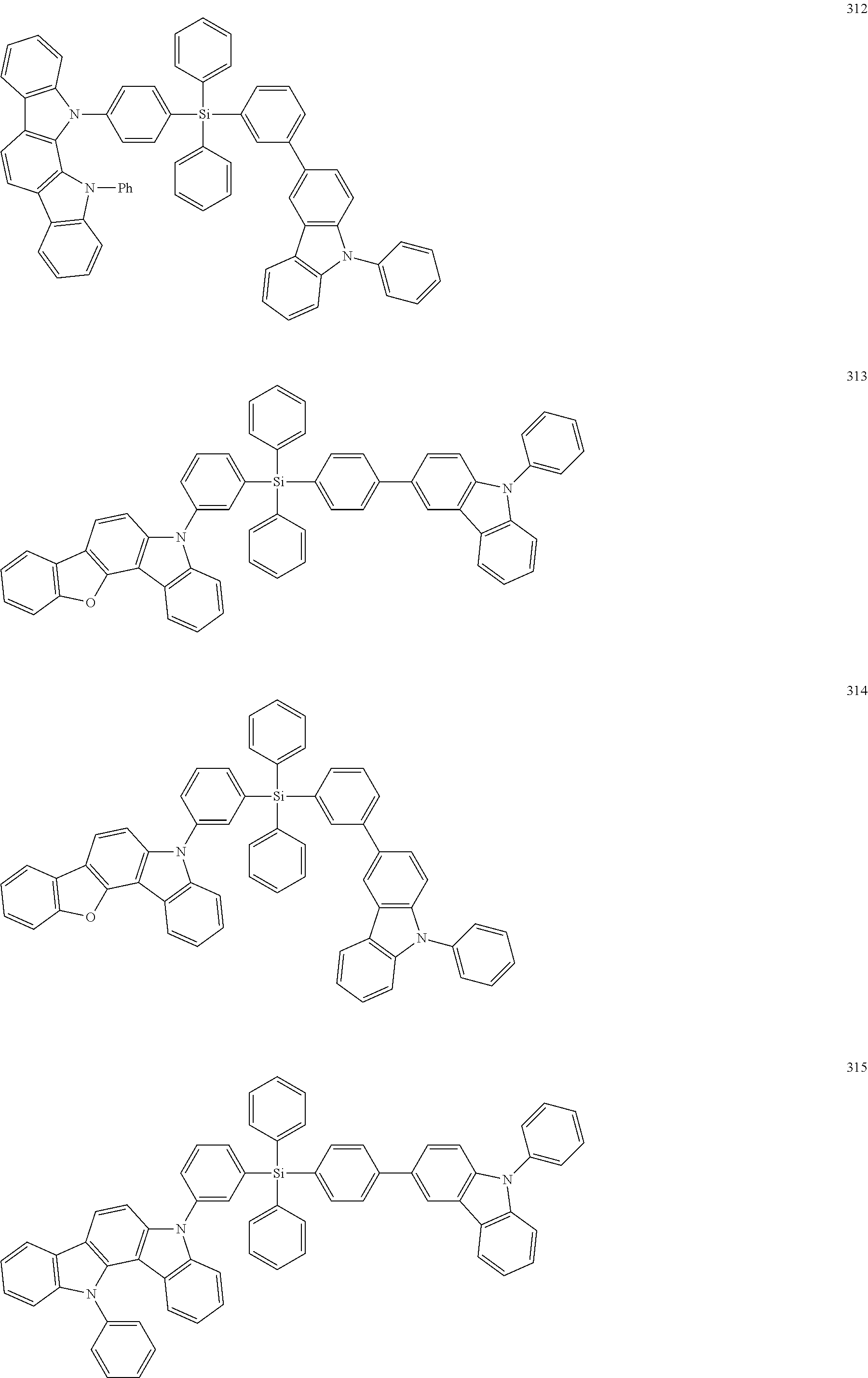 [SMILES: c1ccc(-n2c3ccccc3c3cc(-c4cccc([Si](c5ccccc5)(c5ccccc5)c5ccc(-n6c7ccccc7c7ccc8c9ccccc9n(-c9ccccc9)c8c76)cc5)c4)ccc32)cc1 ; c1ccc(-n2c3ccccc3c3cc(-c4ccc([Si](c5ccccc5)(c5ccccc5)c5cccc(-n6c7ccccc7c7c8oc9ccccc9c8ccc76)c5)cc4)ccc32)cc1 ; c1ccc(-n2c3ccccc3c3cc(-c4cccc([Si](c5ccccc5)(c5ccccc5)c5cccc(-n6c7ccccc7c7c8oc9ccccc9c8ccc76)c5)c4)ccc32)cc1 ; c1ccc(-n2c3ccccc3c3cc(-c4ccc([Si](c5ccccc5)(c5ccccc5)c5cccc(-n6c7ccccc7c7c6ccc6c8ccccc8n(-c8ccccc8)c67)c5)cc4)ccc32)cc1]

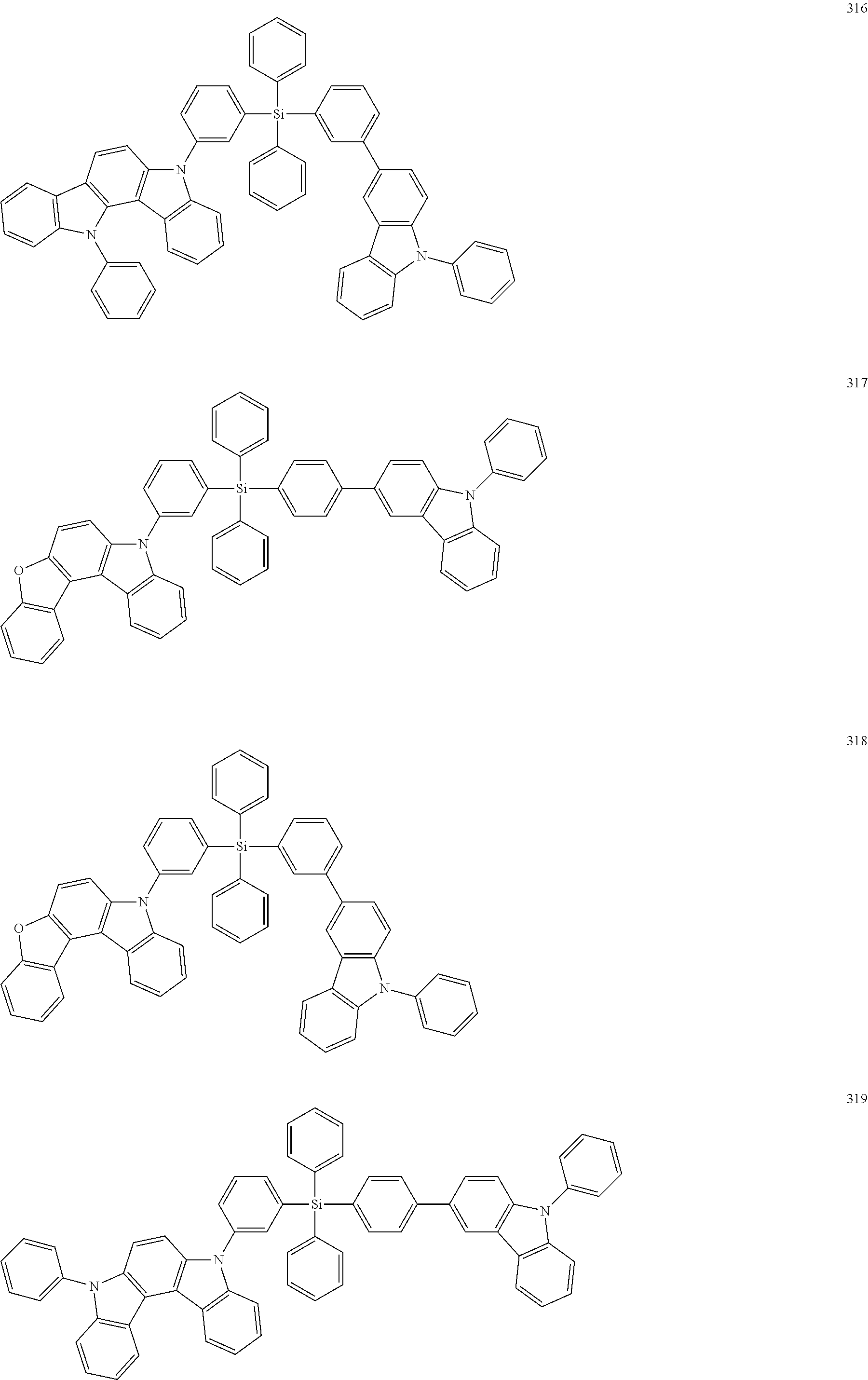 [SMILES: c1ccc(-n2c3ccccc3c3cc(-c4cccc([Si](c5ccccc5)(c5ccccc5)c5cccc(-n6c7ccccc7c7c6ccc6c8ccccc8n(-c8ccccc8)c67)c5)c4)ccc32)cc1 ; c1ccc(-n2c3ccccc3c3cc(-c4ccc([Si](c5ccccc5)(c5ccccc5)c5cccc(-n6c7ccccc7c7c8c(ccc76)oc6ccccc68)c5)cc4)ccc32)cc1 ; c1ccc(-n2c3ccccc3c3cc(-c4cccc([Si](c5ccccc5)(c5ccccc5)c5cccc(-n6c7ccccc7c7c8c(ccc76)oc6ccccc68)c5)c4)ccc32)cc1 ; c1ccc(-n2c3ccccc3c3cc(-c4ccc([Si](c5ccccc5)(c5ccccc5)c5cccc(-n6c7ccccc7c7c8c9ccccc9n(-c9ccccc9)c8ccc76)c5)cc4)ccc32)cc1]

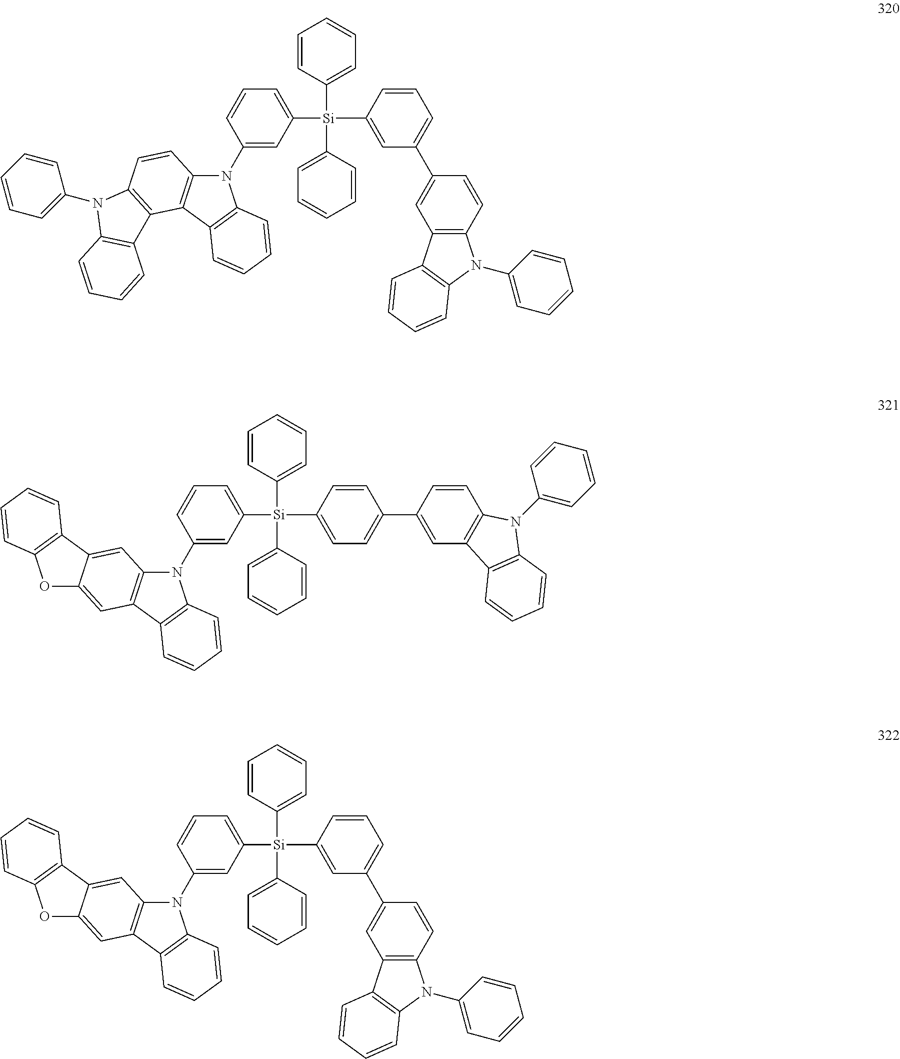 [SMILES: c1ccc(-n2c3ccccc3c3cc(-c4cccc([Si](c5ccccc5)(c5ccccc5)c5cccc(-n6c7ccccc7c7c8c9ccccc9n(-c9ccccc9)c8ccc76)c5)c4)ccc32)cc1 ; c1ccc(-n2c3ccccc3c3cc(-c4ccc([Si](c5ccccc5)(c5ccccc5)c5cccc(-n6c7ccccc7c7cc8oc9ccccc9c8cc76)c5)cc4)ccc32)cc1 ; c1ccc(-n2c3ccccc3c3cc(-c4cccc([Si](c5ccccc5)(c5ccccc5)c5cccc(-n6c7ccccc7c7cc8oc9ccccc9c8cc76)c5)c4)ccc32)cc1]

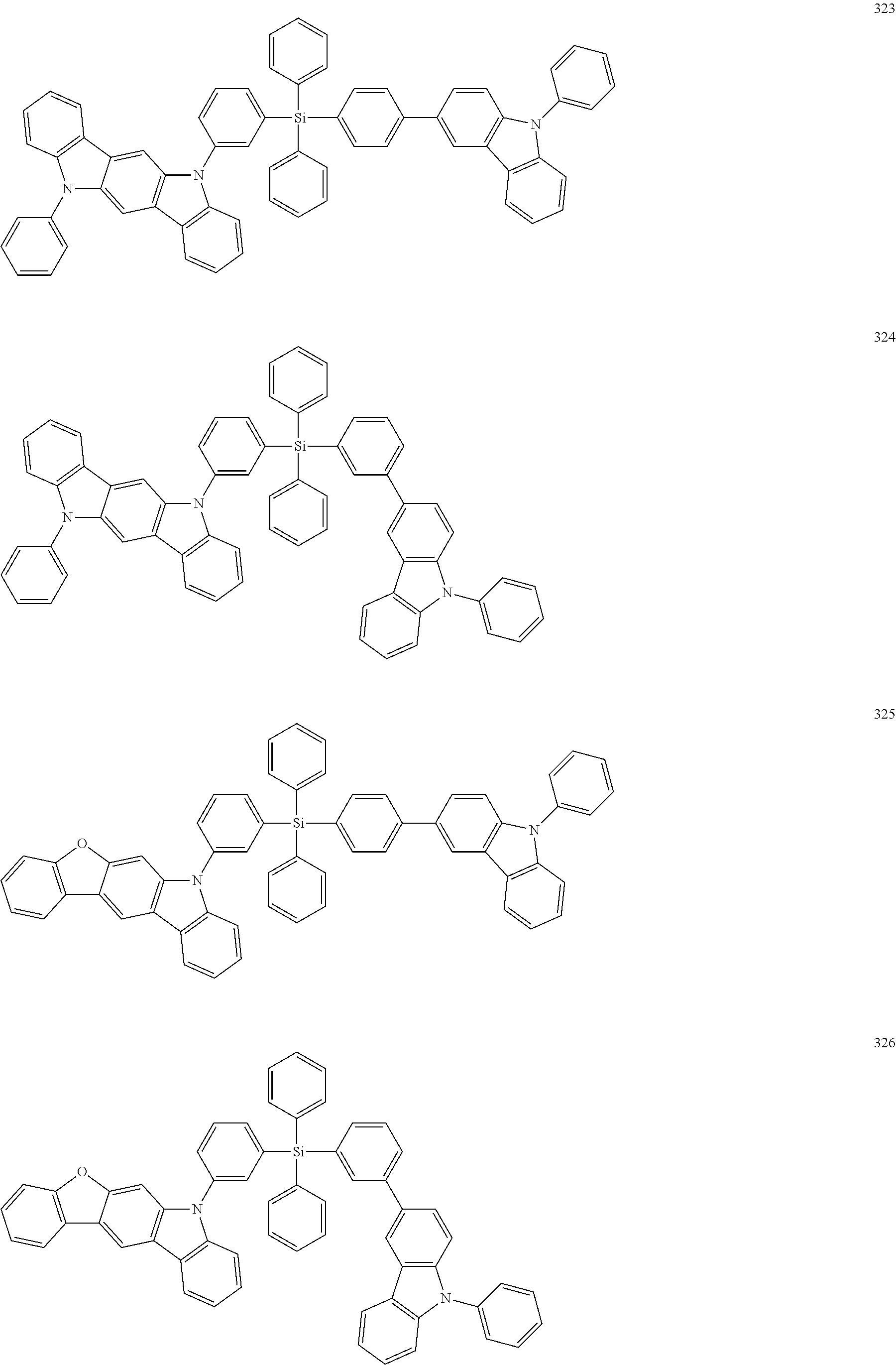 [SMILES: c1ccc(-n2c3ccccc3c3cc(-c4ccc([Si](c5ccccc5)(c5ccccc5)c5cccc(-n6c7ccccc7c7cc8c(cc76)c6ccccc6n8-c6ccccc6)c5)cc4)ccc32)cc1 ; c1ccc(-n2c3ccccc3c3cc(-c4cccc([Si](c5ccccc5)(c5ccccc5)c5cccc(-n6c7ccccc7c7cc8c(cc76)c6ccccc6n8-c6ccccc6)c5)c4)ccc32)cc1 ; c1ccc(-n2c3ccccc3c3cc(-c4ccc([Si](c5ccccc5)(c5ccccc5)c5cccc(-n6c7ccccc7c7cc8c(cc76)oc6ccccc68)c5)cc4)ccc32)cc1 ; c1ccc(-n2c3ccccc3c3cc(-c4cccc([Si](c5ccccc5)(c5ccccc5)c5cccc(-n6c7ccccc7c7cc8c(cc76)oc6ccccc68)c5)c4)ccc32)cc1]

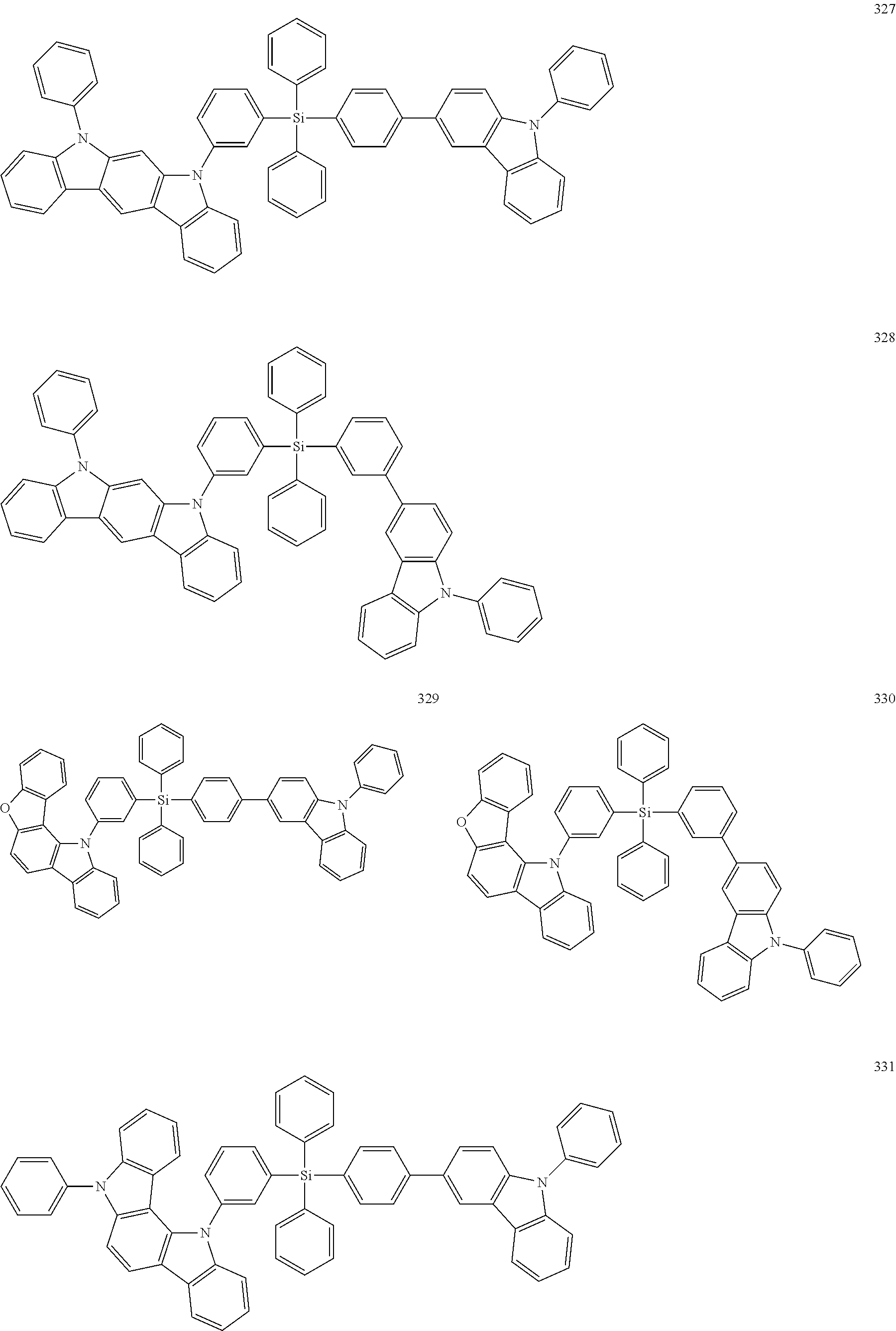

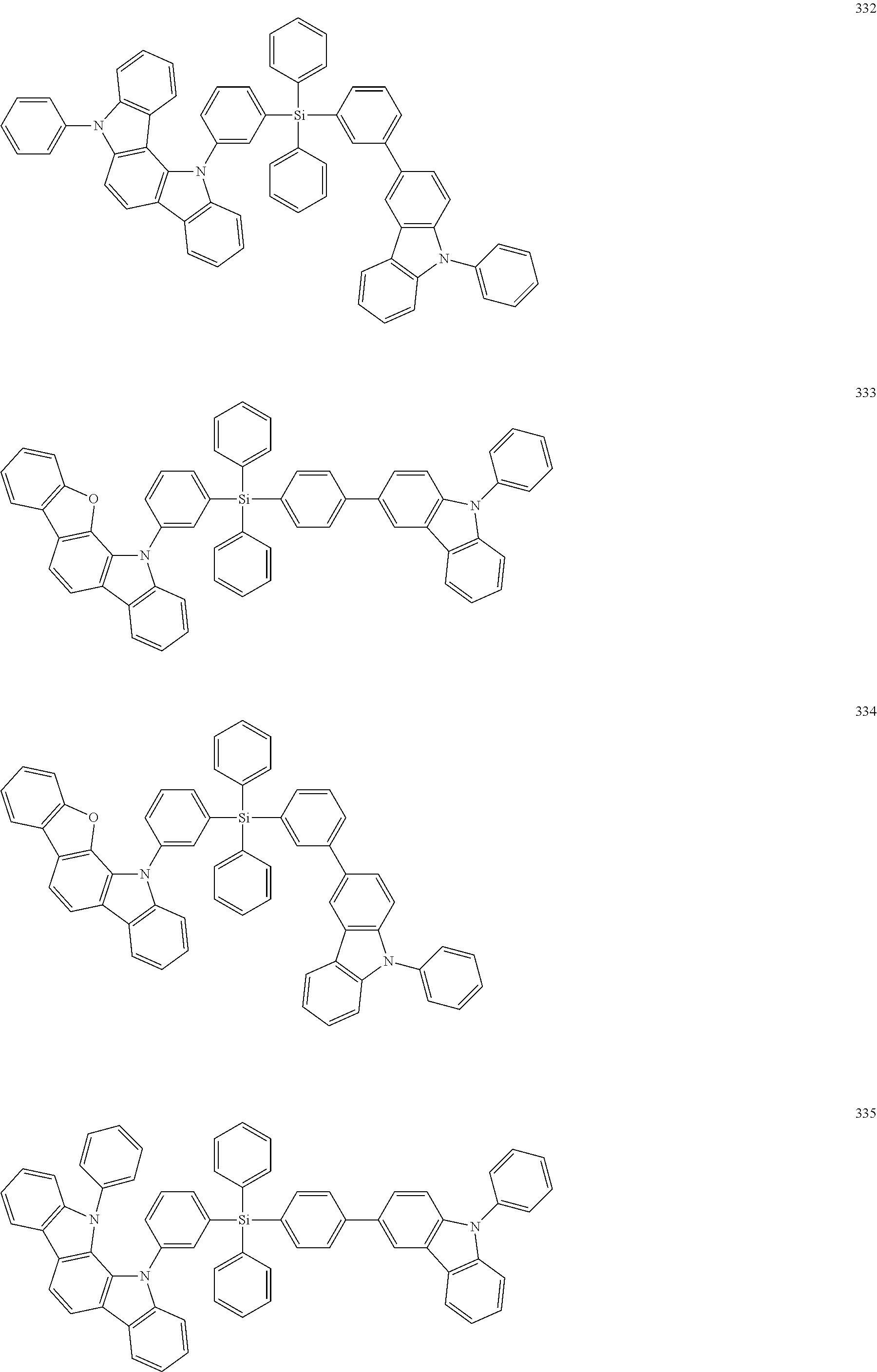 [SMILES: c1ccc(-n2c3ccccc3c3cc(-c4cccc([Si](c5ccccc5)(c5ccccc5)c5cccc(-n6c7ccccc7c7ccc8c(c9ccccc9n8-c8ccccc8)c76)c5)c4)ccc32)cc1 ; c1ccc(-n2c3ccccc3c3cc(-c4ccc([Si](c5ccccc5)(c5ccccc5)c5cccc(-n6c7ccccc7c7ccc8c9ccccc9oc8c76)c5)cc4)ccc32)cc1 ; c1ccc(-n2c3ccccc3c3cc(-c4cccc([Si](c5ccccc5)(c5ccccc5)c5cccc(-n6c7ccccc7c7ccc8c9ccccc9oc8c76)c5)c4)ccc32)cc1 ; c1ccc(-n2c3ccccc3c3cc(-c4ccc([Si](c5ccccc5)(c5ccccc5)c5cccc(-n6c7ccccc7c7ccc8c9ccccc9n(-c9ccccc9)c8c76)c5)cc4)ccc32)cc1]

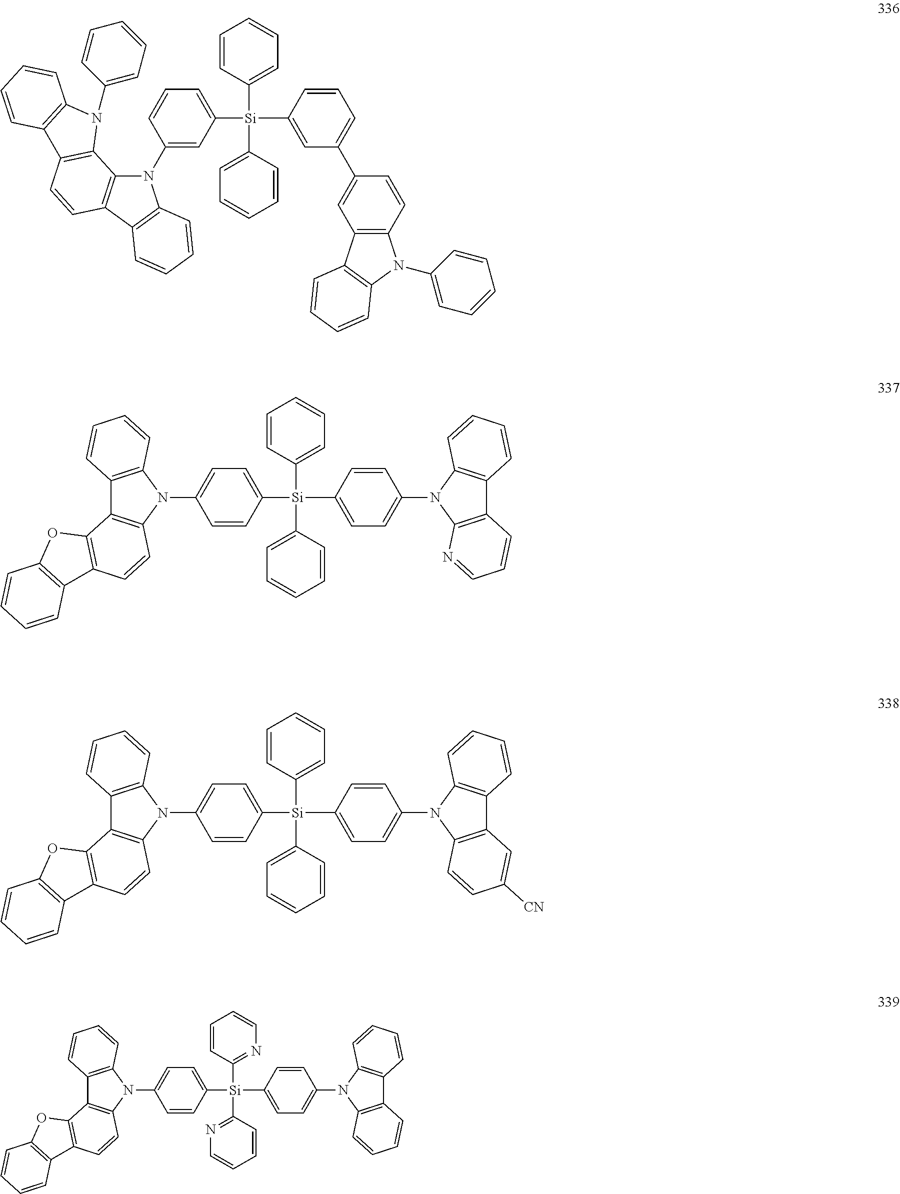 [SMILES: c1ccc(-n2c3ccccc3c3cc(-c4cccc([Si](c5ccccc5)(c5ccccc5)c5cccc(-n6c7ccccc7c7ccc8c9ccccc9n(-c9ccccc9)c8c76)c5)c4)ccc32)cc1 ; c1ccc([Si](c2ccccc2)(c2ccc(-n3c4ccccc4c4c5oc6ccccc6c5ccc43)cc2)c2ccc(-n3c4ccccc4c4cccnc43)cc2)cc1 ; N#Cc1ccc2c(c1)c1ccccc1n2-c1ccc([Si](c2ccccc2)(c2ccccc2)c2ccc(-n3c4ccccc4c4c5oc6ccccc6c5ccc43)cc2)cc1 ; c1ccc([Si](c2ccc(-n3c4ccccc4c4ccccc43)cc2)(c2ccc(-n3c4ccccc4c4c5oc6ccccc6c5ccc43)cc2)c2ccccn2)nc1]

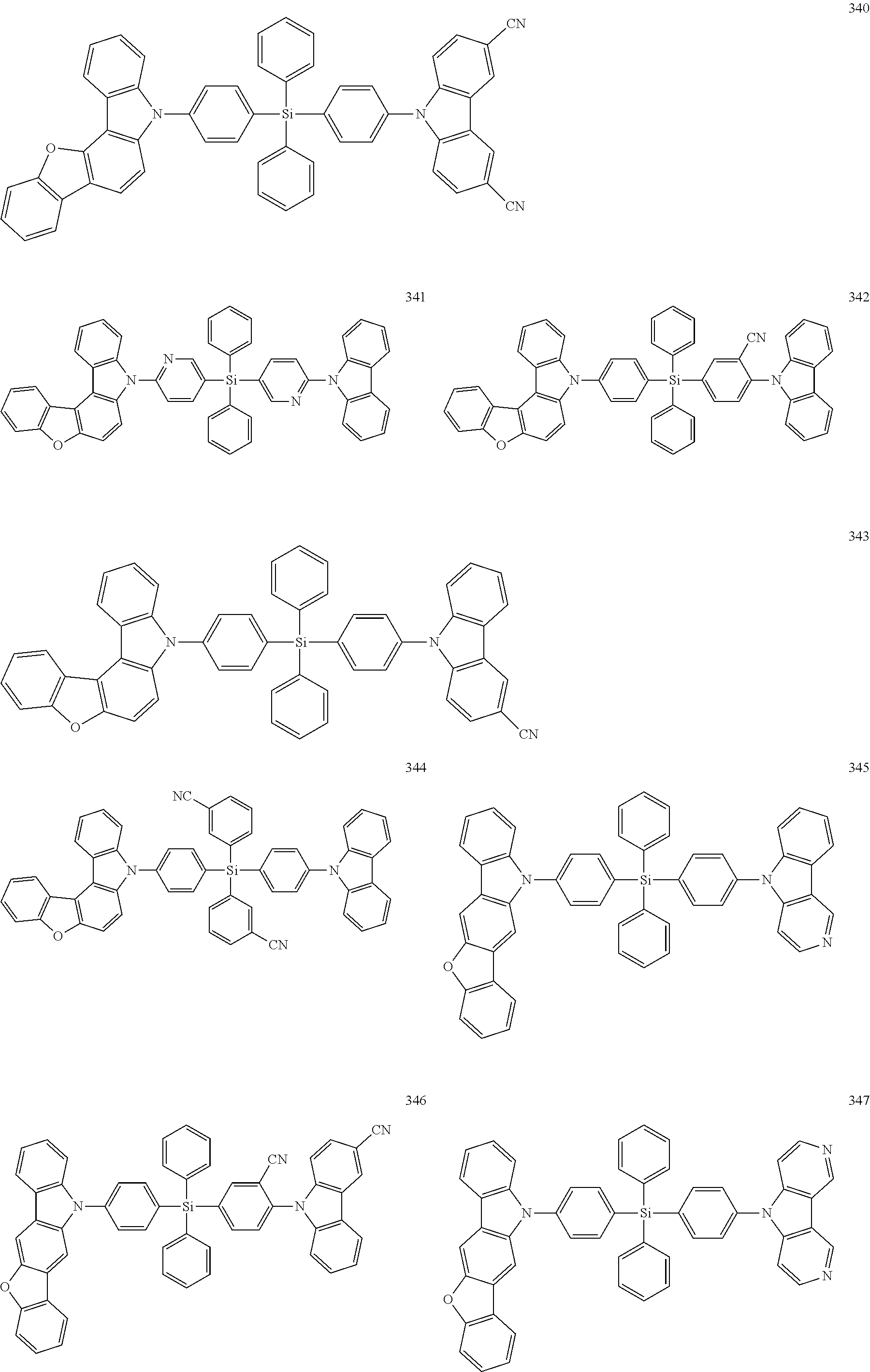

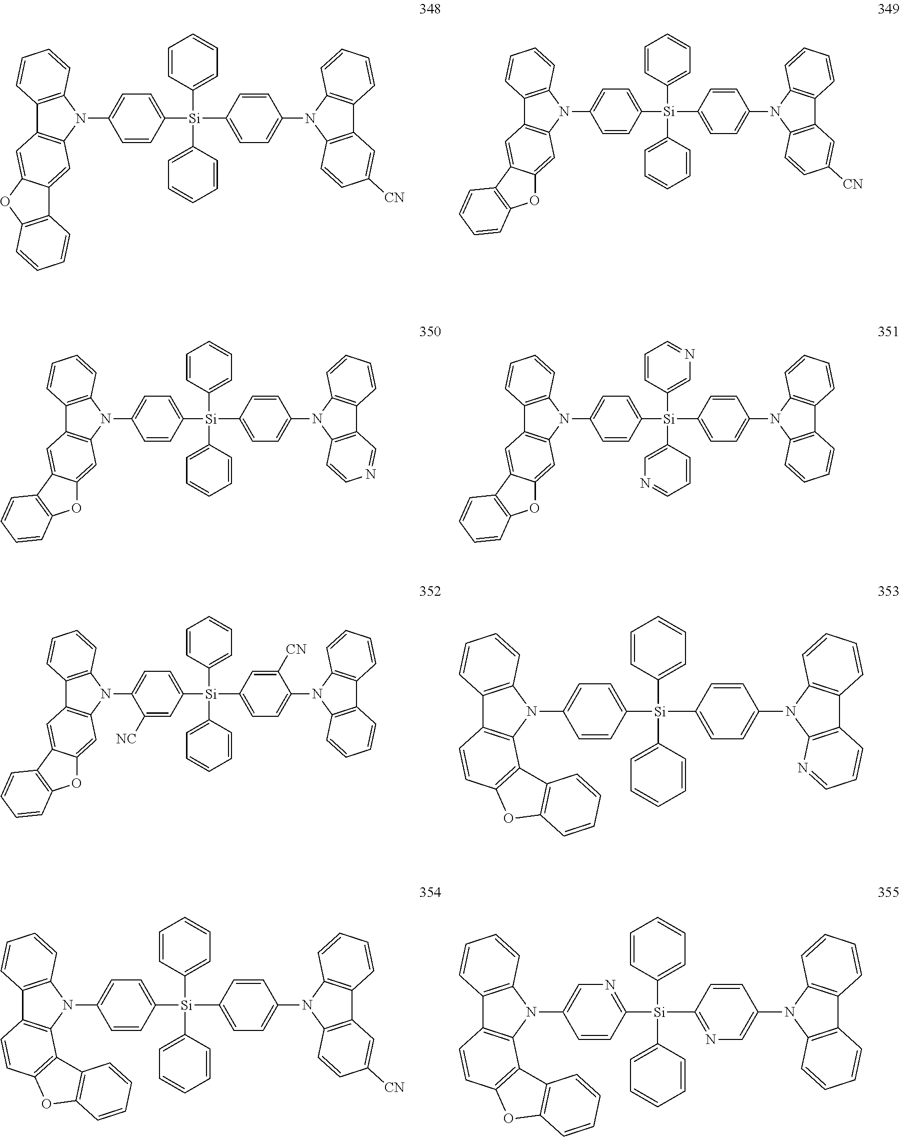

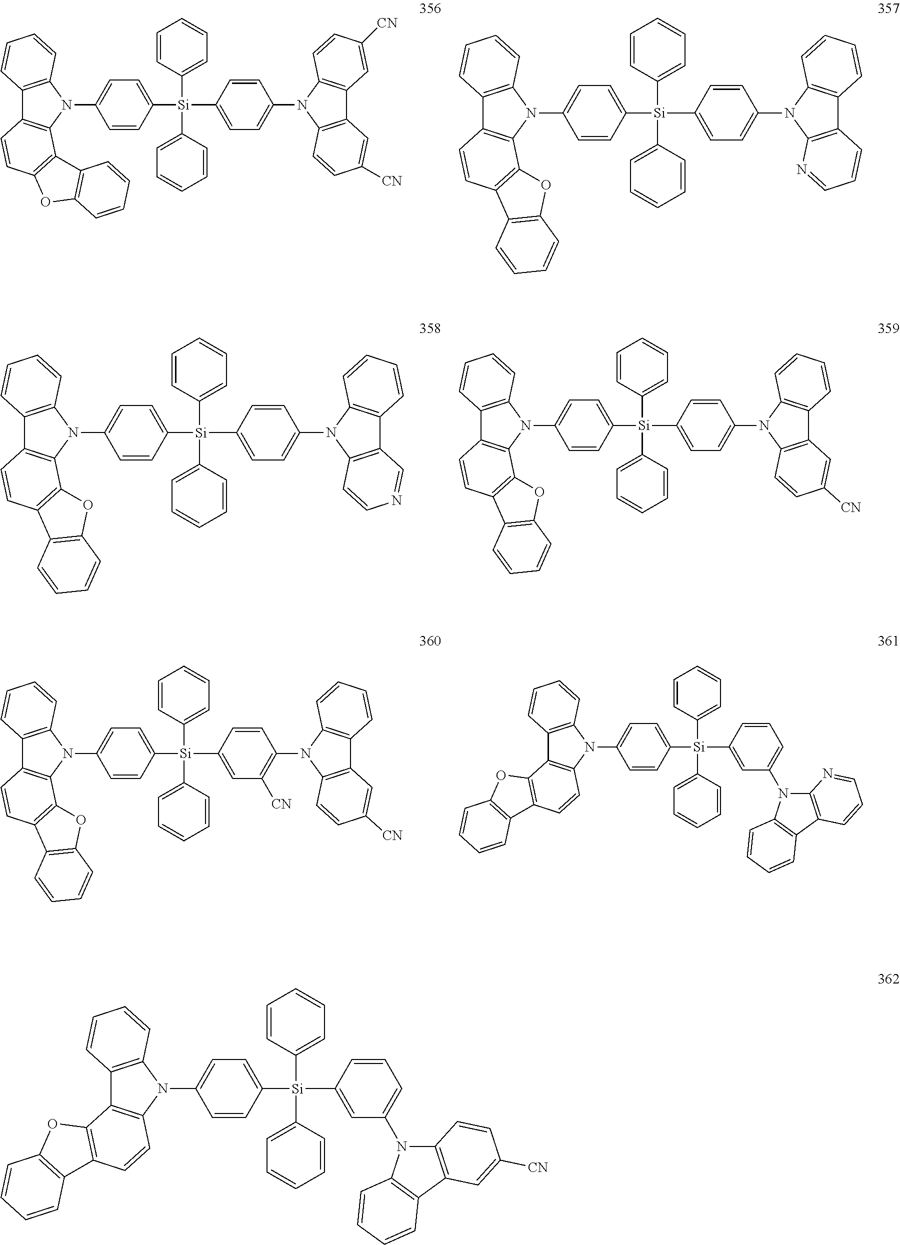

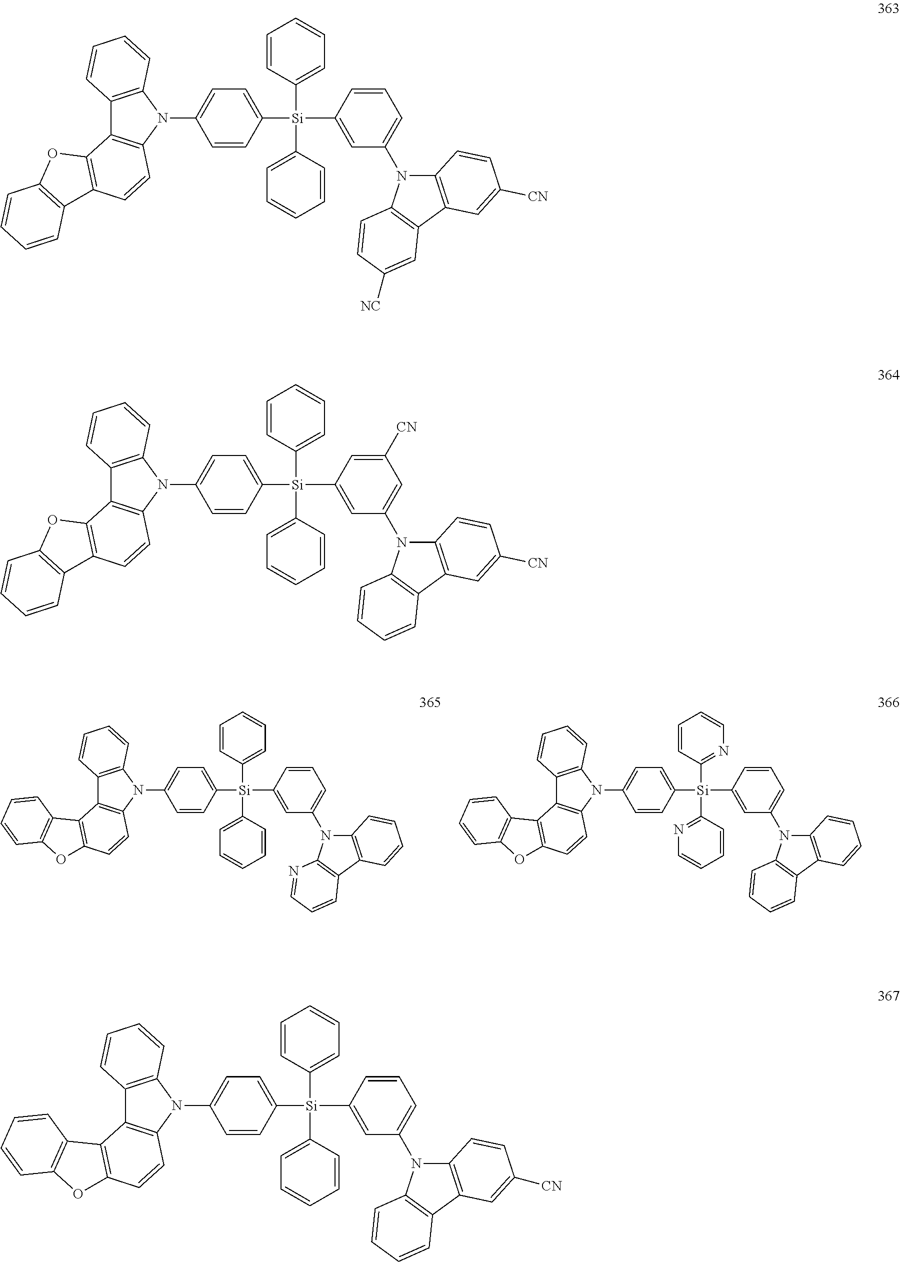

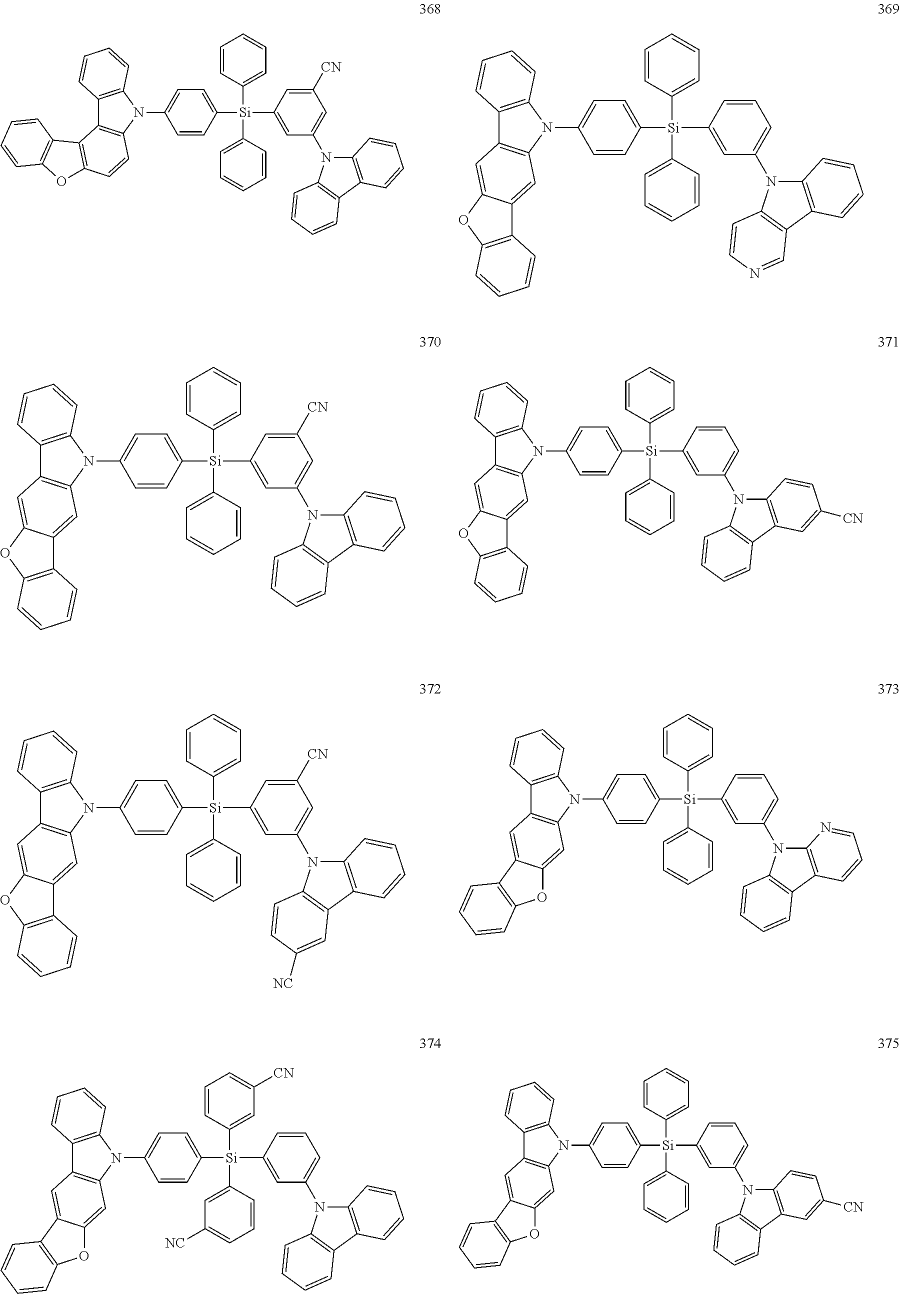

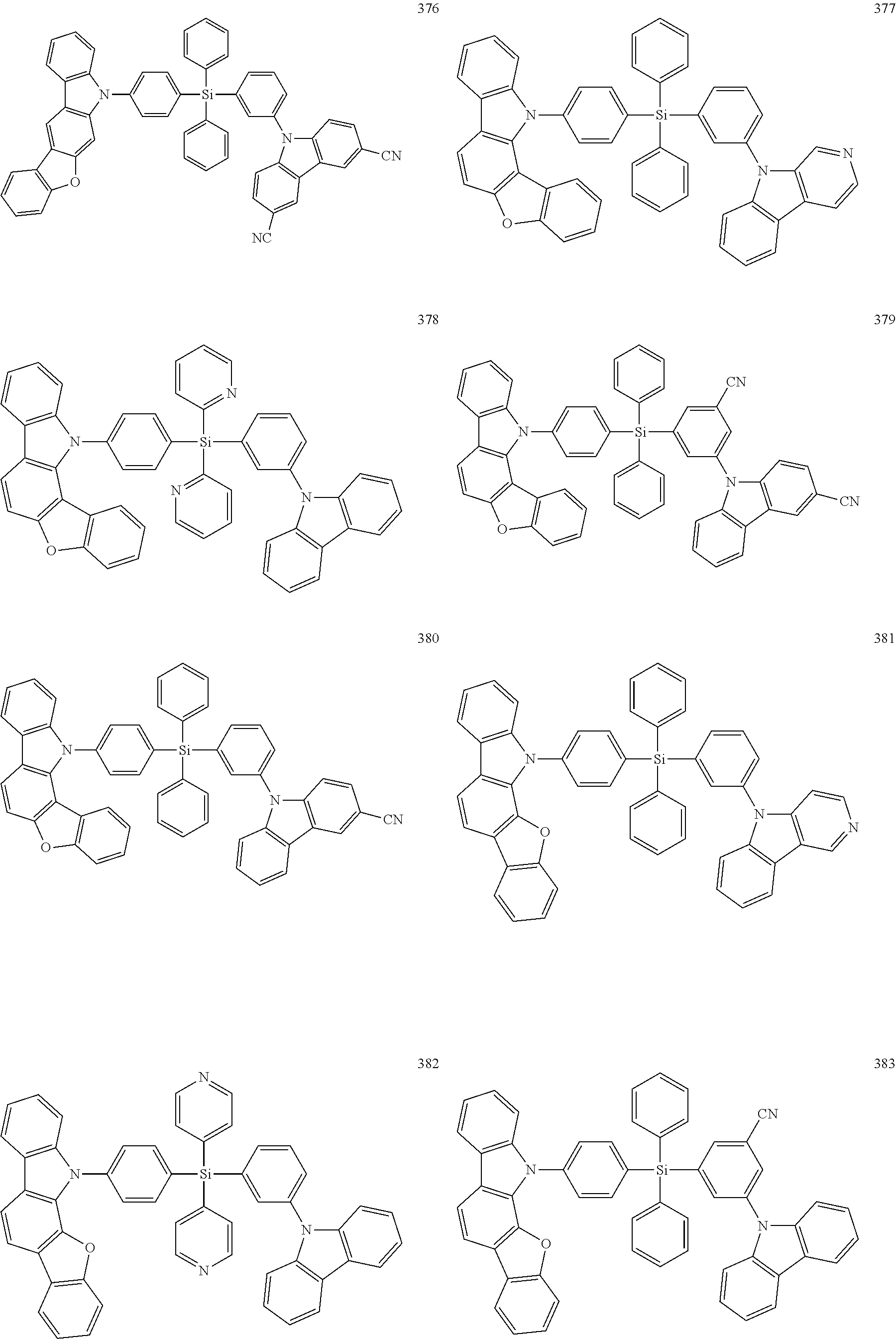

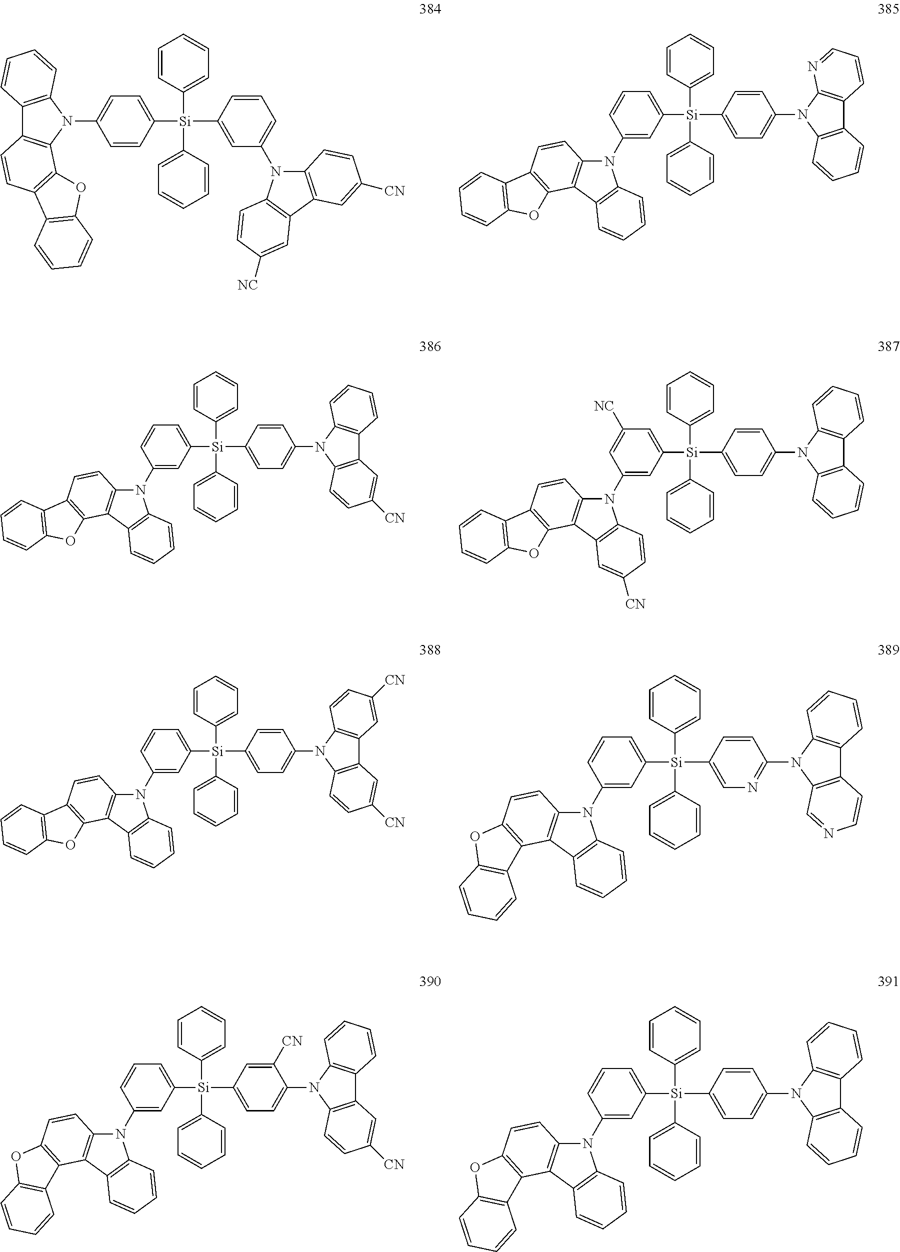

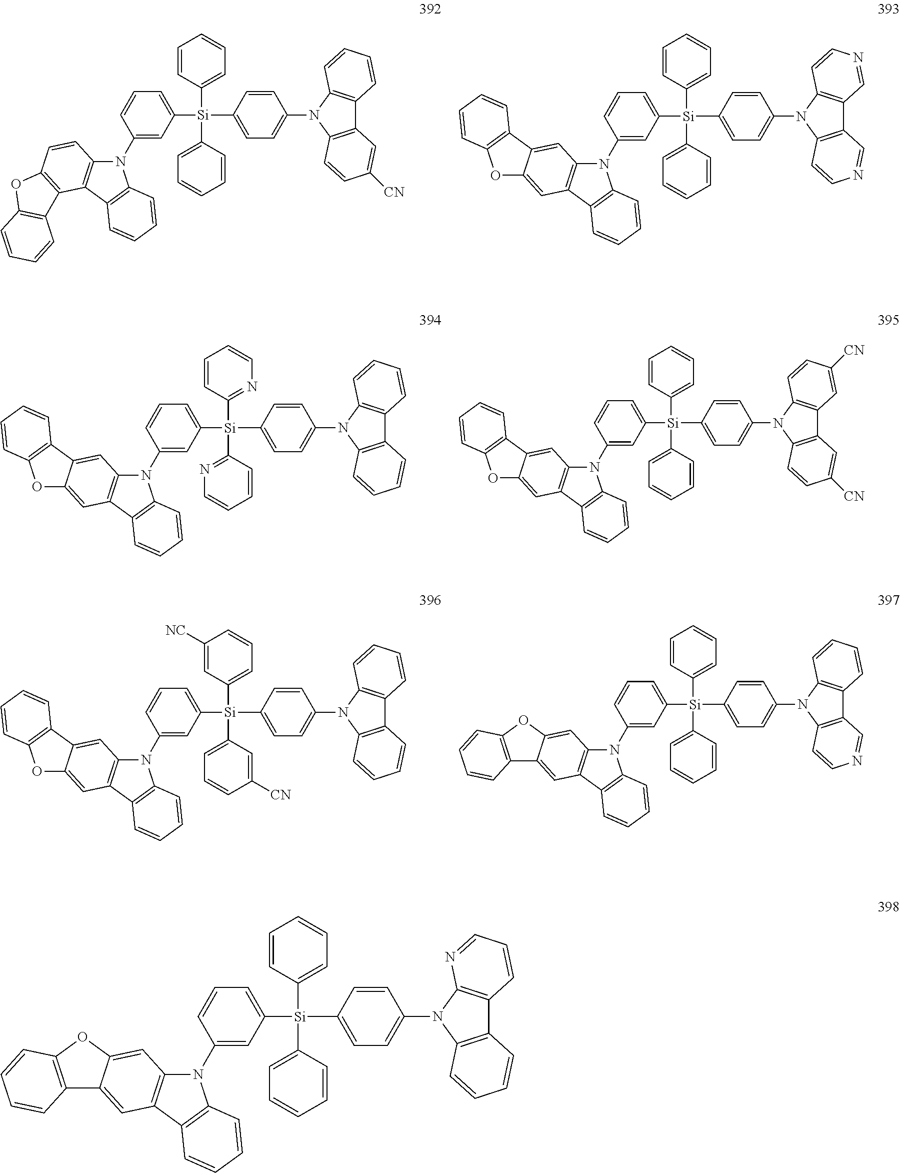

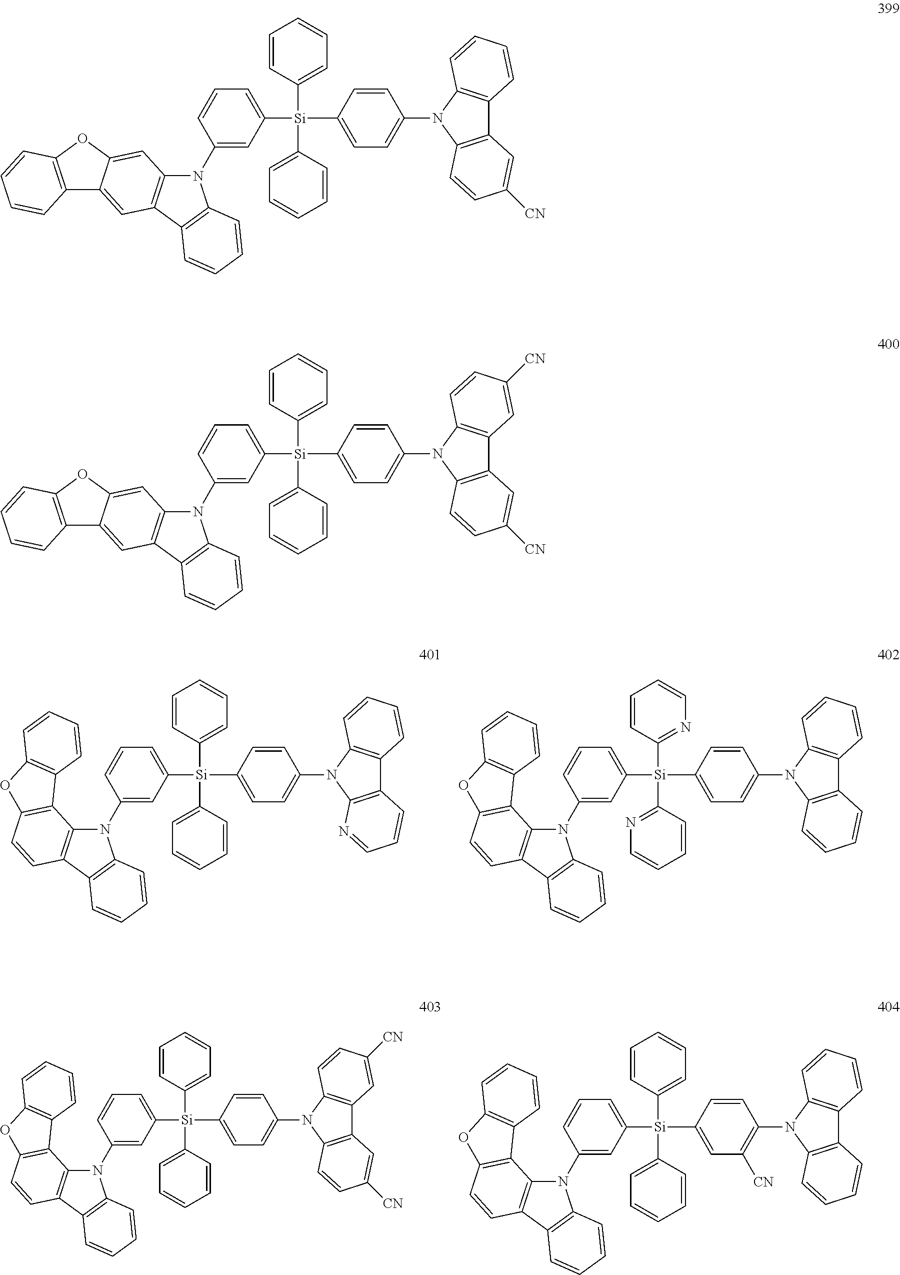

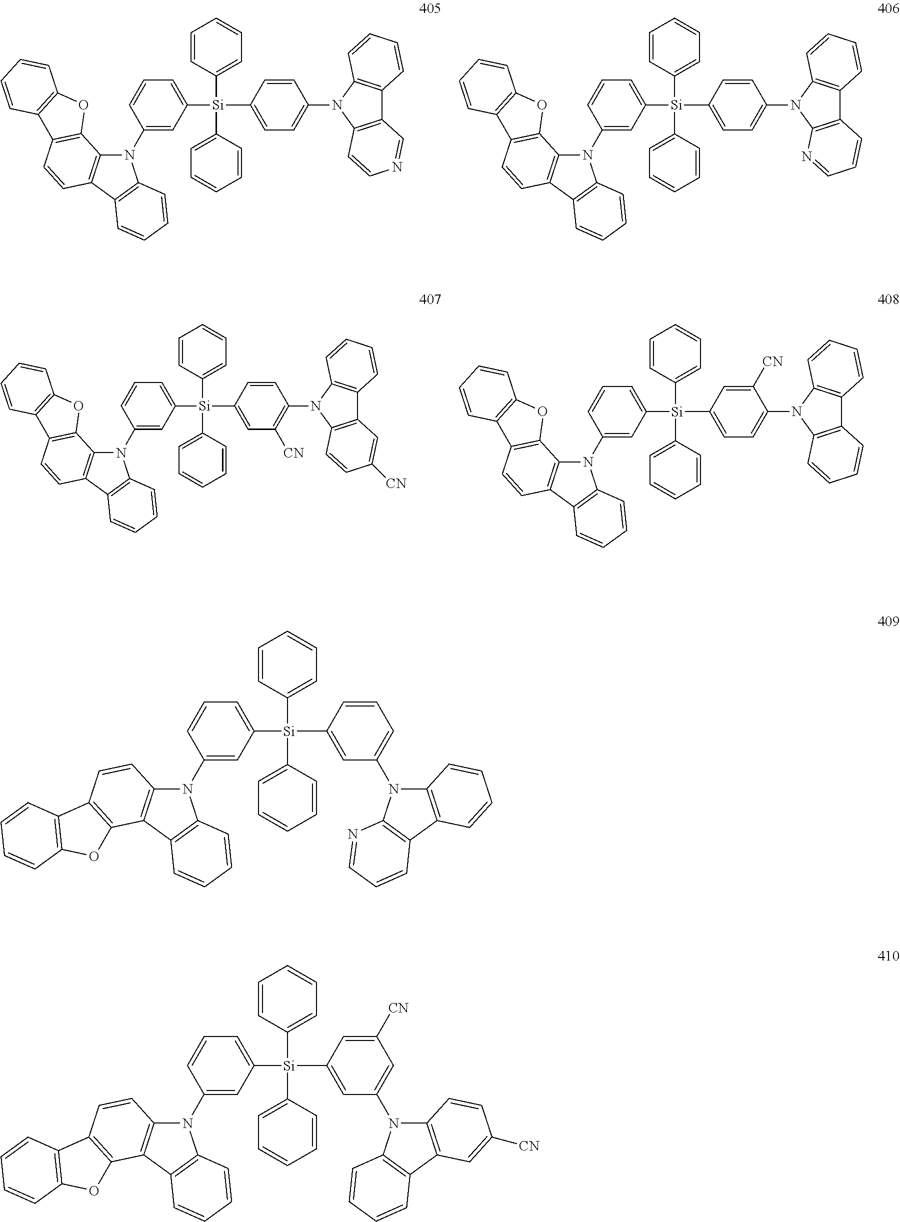

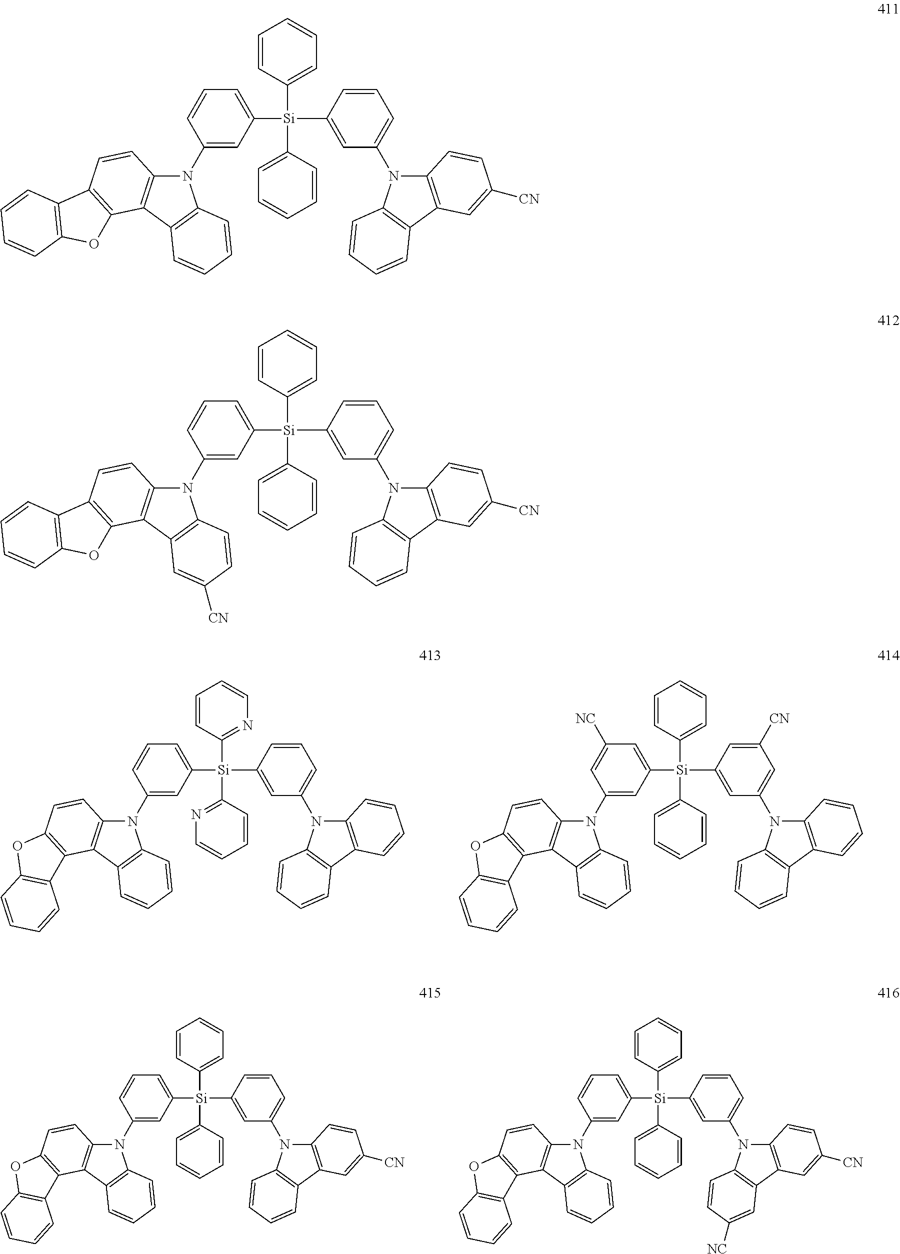

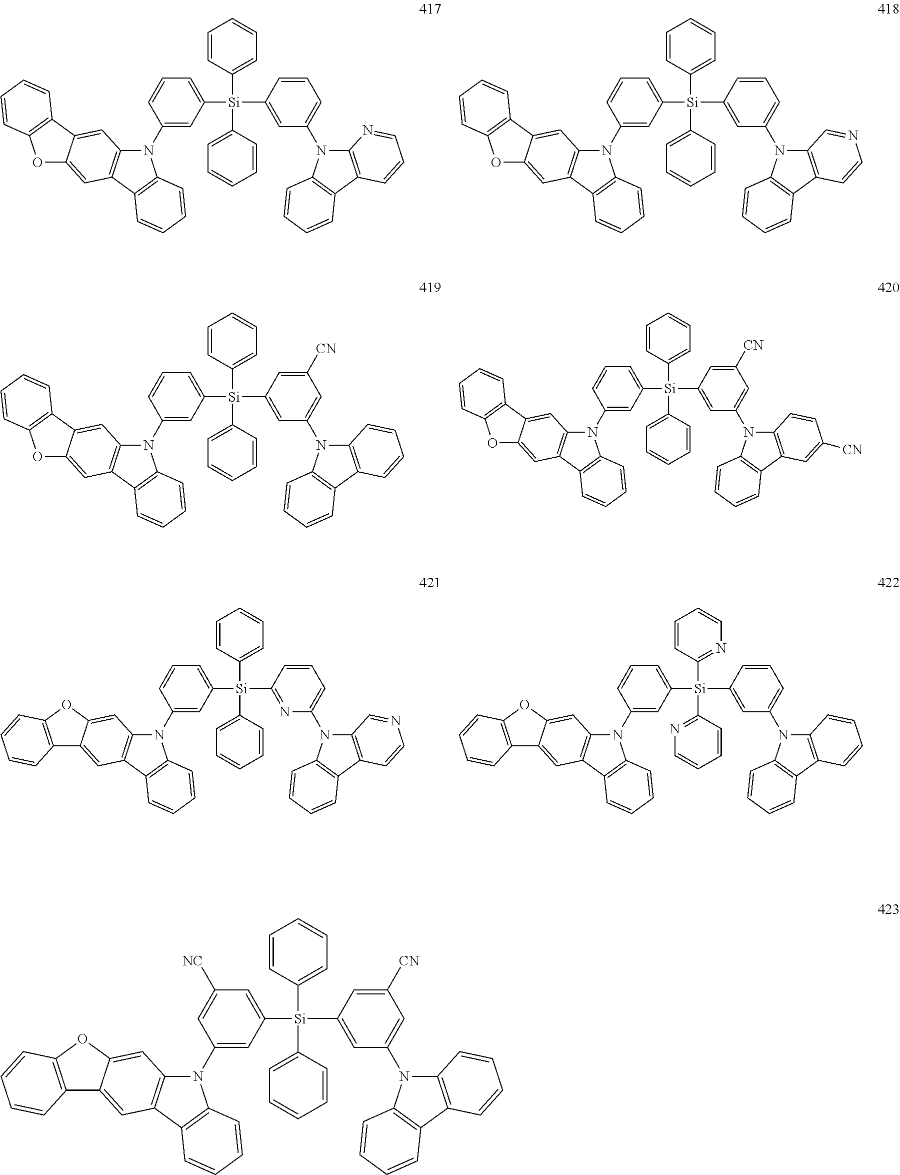

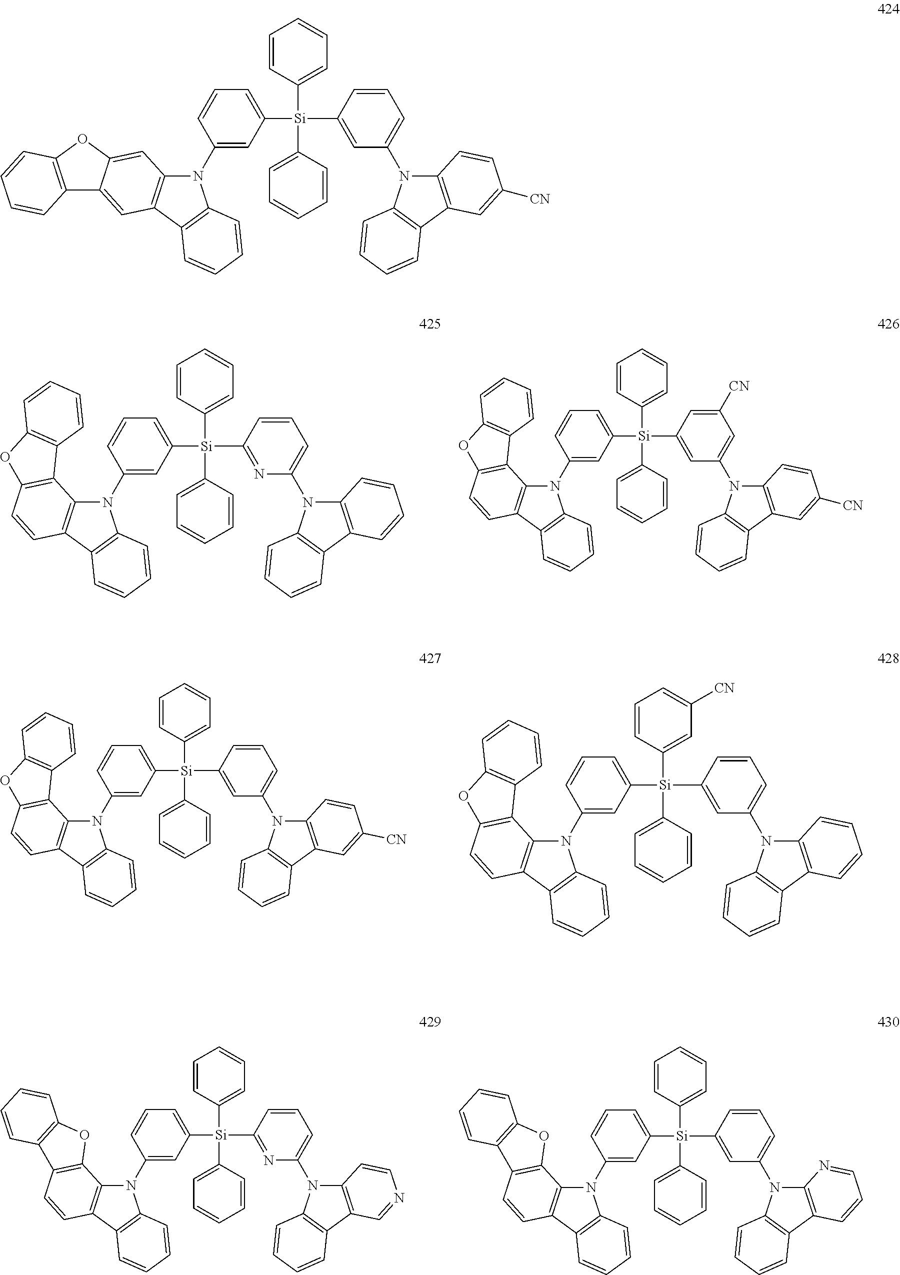

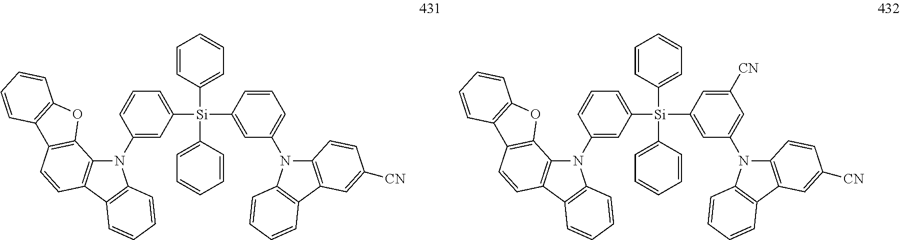 [SMILES: N#Cc1ccc2c(c1)c1ccccc1n2-c1cccc([Si](c2ccccc2)(c2ccccc2)c2cccc(-n3c4ccccc4c4ccc5c6ccccc6oc5c43)c2)c1 ; N#Cc1cc(-n2c3ccccc3c3cc(C#N)ccc32)cc([Si](c2ccccc2)(c2ccccc2)c2cccc(-n3c4ccccc4c4ccc5c6ccccc6oc5c43)c2)c1]

wherein in Compounds 1 to 432,

“Ph” represents a phenyl group.

In some embodiments, the hole transporting host may include o-CBP or mCP:

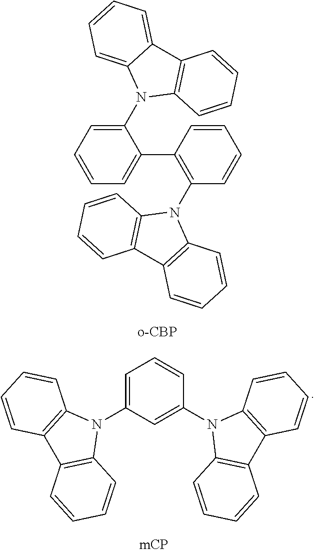 [SMILES: c1ccc(-n2c3ccccc3c3ccccc32)c(-c2ccccc2-n2c3ccccc3c3ccccc32)c1 ; c1cc(-n2c3ccccc3c3ccccc32)cc(-n2c3ccccc3c3ccccc32)c1]

In some embodiments, the host may be a fluorescent host, and the fluorescent host may be represented by, for example, one of Formulae FH-1 to FH-4.

In some embodiments, the fluorescent host may be represented by Formula FH-1:

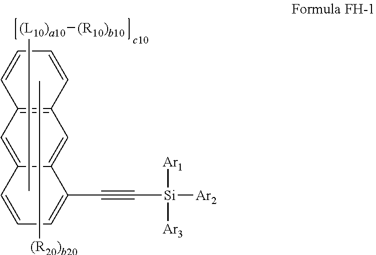

wherein, in Formula FH-1,

Ar₁ to Ar₃ may each independently be a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₃-C₆₀ aryl group, a substituted or unsubstituted C₃-C₆₀ aryloxy group, a substituted or unsubstituted C₃-C₆₀ arylthio group, a substituted or unsubstituted C₁-C₆₀ heteroaryl group, a substituted or unsubstituted monovalent non-aromatic condensed polycyclic group, a substituted or unsubstituted monovalent non-aromatic condensed heteropolycyclic group, —N(Q₁)(Q₂), —Si(Q₃)(Q₄)(Q₅), —B(Q₆)(Q₇), or —P(═O)(Q₈)(Q₉), and

at least one of Ar₁ to Ar₃ may be a substituted or unsubstituted C₃-C₆₀ aryl group, a substituted or unsubstituted C₁-C₆₀ heteroaryl group, a substituted or unsubstituted monovalent non-aromatic condensed polycyclic group, or a substituted or unsubstituted monovalent non-aromatic condensed heteropolycyclic group,

L₁₀ may be a substituted or unsubstituted C₅-C₃₀ carbocyclic group or a substituted or unsubstituted C₁-C₃₀ heterocyclic group,

a10 may be an integer from 0 to 3, and when a10 is 2 or greater, at least two L₁₀ (s) may be identical to or different from each other,

R₁₀ and R₂₀ may each independently be hydrogen, deuterium, —F, —Cl, —Br, —I, —SF₅, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁ heterocycloalkyl group, a substituted or unsubstituted C₃-C₁₀ cycloalkenyl group, a substituted or unsubstituted C₁-C₁₀ heterocycloalkenyl group, a substituted or unsubstituted C₃-C₆₀ aryl group, a substituted or unsubstituted C₃-C₆₀ aryloxy group, a substituted or unsubstituted C₃-C₆₀ arylthio group, a substituted or unsubstituted C₁-C₆₀ heteroaryl group, a substituted or unsubstituted monovalent non-aromatic condensed polycyclic group, a substituted or unsubstituted monovalent non-aromatic condensed heteropolycyclic group, —N(Q₁)(Q₂), —Si(Q₃)(Q₄)(Q₅), —B(Q₆)(Q₇), or —P(═O)(Q₈)(Q₉),

b10 and b20 may each independently be an integer from 1 to 8,

when b10 is 2 or greater, at least two Rio(s) may be identical to or different from each other, and when b20 is 2 or greater, at least two R₂₀(s) may be identical to or different from each other,

c10 may be an integer from 1 to 9, and

when c10 is 2 or greater, at least two —[(L₁₀)ₐ₁₀—(R₁₀)_{b10}](s) may be identical to or different from each other.

In some embodiments, the fluorescent host represented by Formula FH-1 may be Group FH1, but embodiments are not limited thereto:

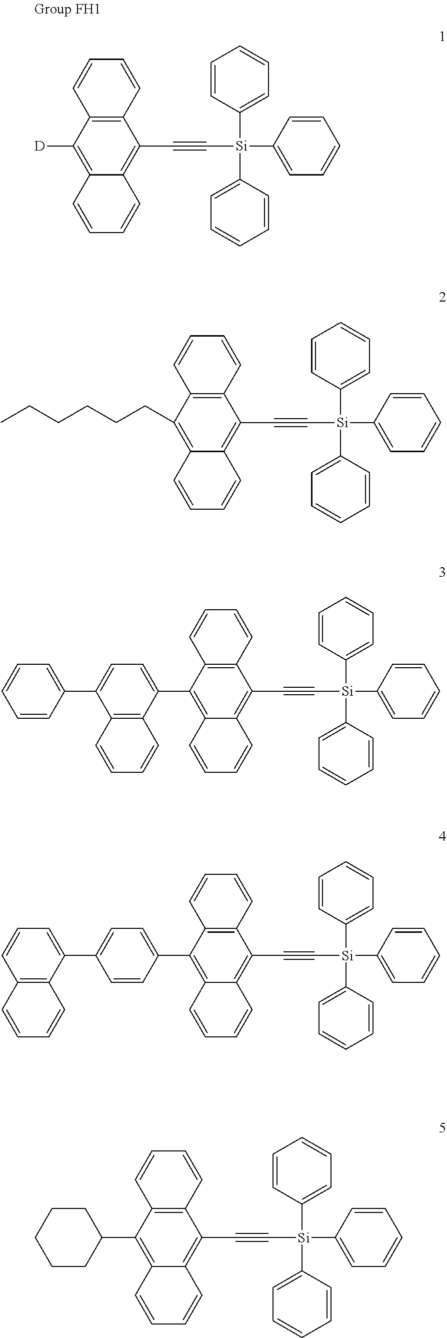 [SMILES: [2H]c1c2ccccc2c(C#C[Si](c2ccccc2)(c2ccccc2)c2ccccc2)c2ccccc12 ; CCCCCCc1c2ccccc2c(C#C[Si](c2ccccc2)(c2ccccc2)c2ccccc2)c2ccccc12 ; C(#C[Si](c1ccccc1)(c1ccccc1)c1ccccc1)c1c2ccccc2c(-c2ccc(-c3ccccc3)c3ccccc23)c2ccccc12 ; C(#C[Si](c1ccccc1)(c1ccccc1)c1ccccc1)c1c2ccccc2c(-c2ccc(-c3cccc4ccccc34)cc2)c2ccccc12 ; C(#C[Si](c1ccccc1)(c1ccccc1)c1ccccc1)c1c2ccccc2c(C2CCCCC2)c2ccccc12]

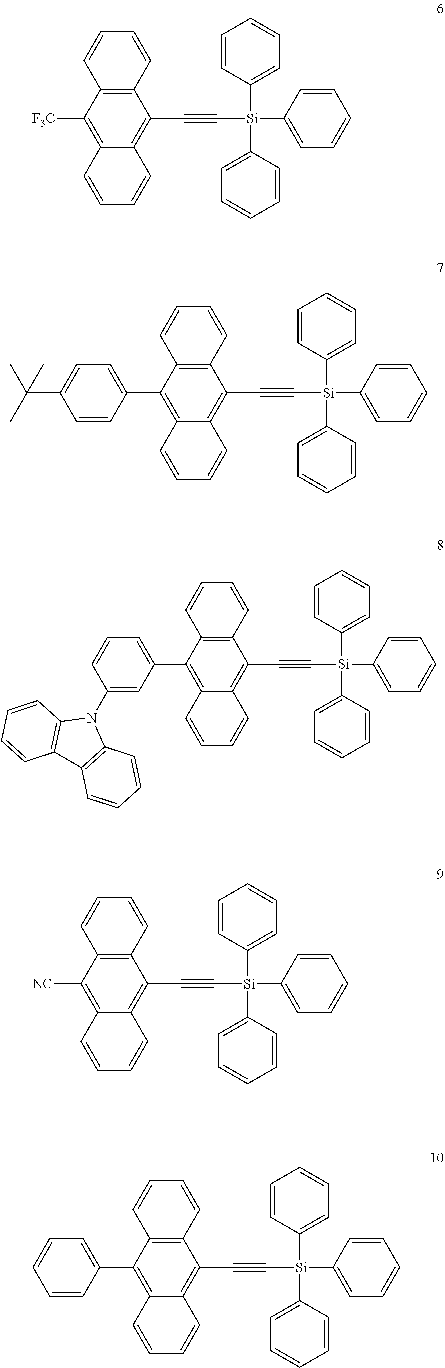 [SMILES: FC(F)(F)c1c2ccccc2c(C#C[Si](c2ccccc2)(c2ccccc2)c2ccccc2)c2ccccc12 ; CC(C)(C)c1ccc(-c2c3ccccc3c(C#C[Si](c3ccccc3)(c3ccccc3)c3ccccc3)c3ccccc23)cc1 ; C(#C[Si](c1ccccc1)(c1ccccc1)c1ccccc1)c1c2ccccc2c(-c2cccc(-n3c4ccccc4c4ccccc43)c2)c2ccccc12 ; N#Cc1c2ccccc2c(C#C[Si](c2ccccc2)(c2ccccc2)c2ccccc2)c2ccccc12 ; C(#C[Si](c1ccccc1)(c1ccccc1)c1ccccc1)c1c2ccccc2c(-c2ccccc2)c2ccccc12]

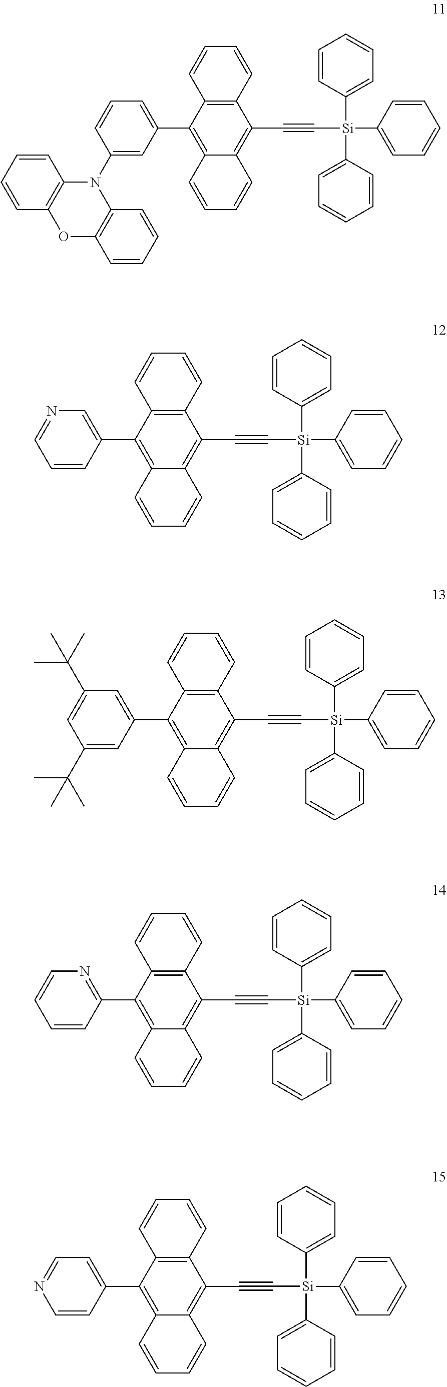 [SMILES: C(#C[Si](c1ccccc1)(c1ccccc1)c1ccccc1)c1c2ccccc2c(-c2cccc(N3c4ccccc4Oc4ccccc43)c2)c2ccccc12 ; C(#C[Si](c1ccccc1)(c1ccccc1)c1ccccc1)c1c2ccccc2c(-c2cccnc2)c2ccccc12 ; CC(C)(C)c1cc(-c2c3ccccc3c(C#C[Si](c3ccccc3)(c3ccccc3)c3ccccc3)c3ccccc23)cc(C(C)(C)C)c1 ; C(#C[Si](c1ccccc1)(c1ccccc1)c1ccccc1)c1c2ccccc2c(-c2ccccn2)c2ccccc12 ; C(#C[Si](c1ccccc1)(c1ccccc1)c1ccccc1)c1c2ccccc2c(-c2ccncc2)c2ccccc12]

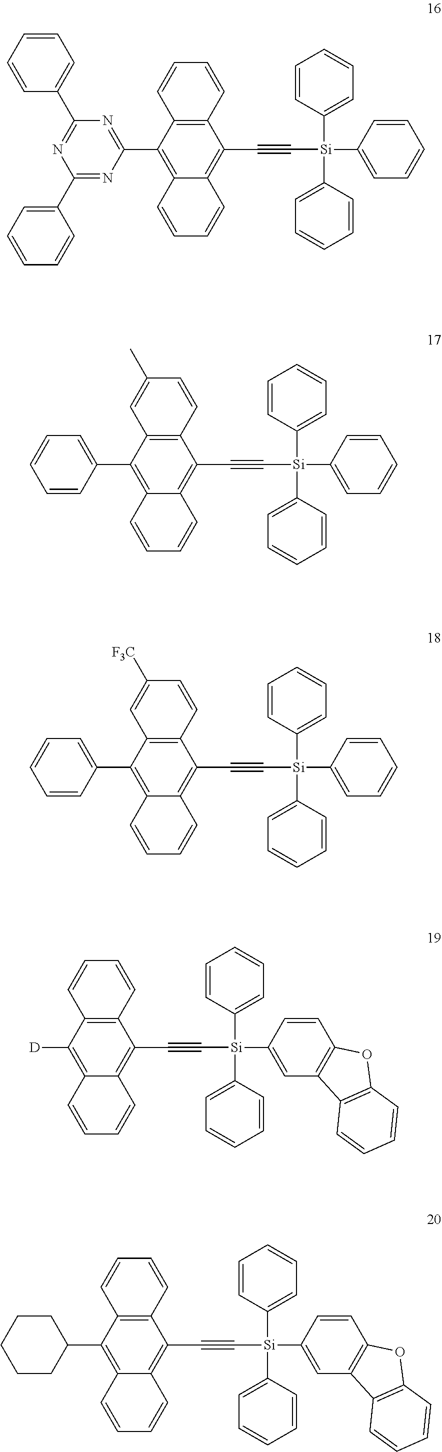 [SMILES: C(#C[Si](c1ccccc1)(c1ccccc1)c1ccccc1)c1c2ccccc2c(-c2nc(-c3ccccc3)nc(-c3ccccc3)n2)c2ccccc12 ; Cc1ccc2c(C#C[Si](c3ccccc3)(c3ccccc3)c3ccccc3)c3ccccc3c(-c3ccccc3)c2c1 ; FC(F)(F)c1ccc2c(C#C[Si](c3ccccc3)(c3ccccc3)c3ccccc3)c3ccccc3c(-c3ccccc3)c2c1 ; [2H]c1c2ccccc2c(C#C[Si](c2ccccc2)(c2ccccc2)c2ccc3oc4ccccc4c3c2)c2ccccc12 ; C(#C[Si](c1ccccc1)(c1ccccc1)c1ccc2oc3ccccc3c2c1)c1c2ccccc2c(C2CCCCC2)c2ccccc12]

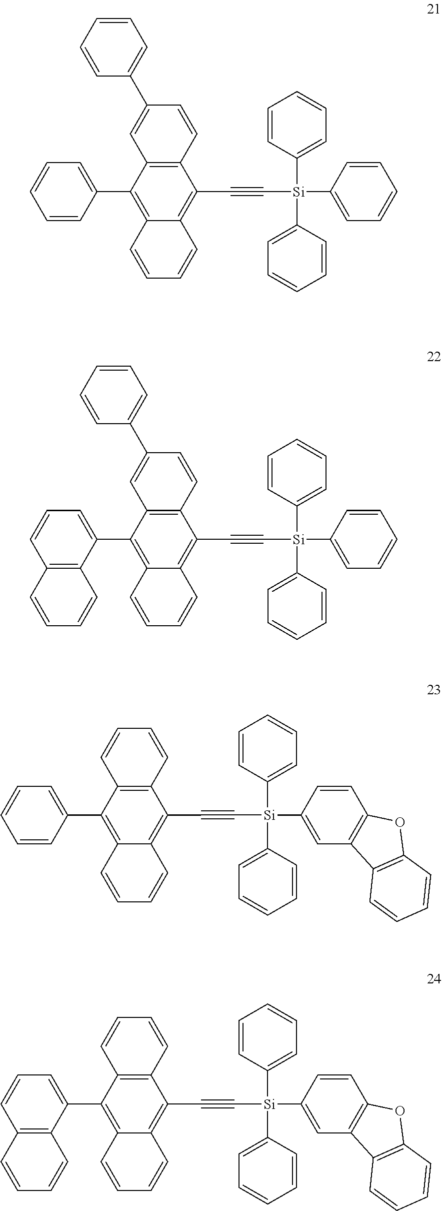 [SMILES: C(#C[Si](c1ccccc1)(c1ccccc1)c1ccccc1)c1c2ccccc2c(-c2ccccc2)c2cc(-c3ccccc3)ccc12 ; C(#C[Si](c1ccccc1)(c1ccccc1)c1ccccc1)c1c2ccccc2c(-c2cccc3ccccc23)c2cc(-c3ccccc3)ccc12 ; C(#C[Si](c1ccccc1)(c1ccccc1)c1ccc2oc3ccccc3c2c1)c1c2ccccc2c(-c2ccccc2)c2ccccc12 ; C(#C[Si](c1ccccc1)(c1ccccc1)c1ccc2oc3ccccc3c2c1)c1c2ccccc2c(-c2cccc3ccccc23)c2ccccc12]

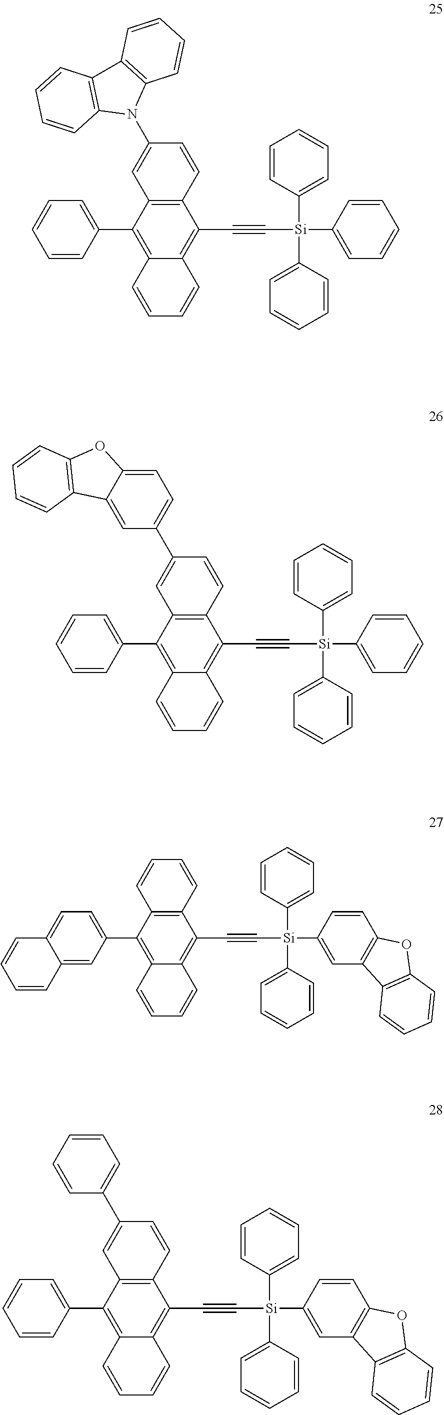 [SMILES: C(#C[Si](c1ccccc1)(c1ccccc1)c1ccccc1)c1c2ccccc2c(-c2ccccc2)c2cc(-n3c4ccccc4c4ccccc43)ccc12 ; C(#C[Si](c1ccccc1)(c1ccccc1)c1ccccc1)c1c2ccccc2c(-c2ccccc2)c2cc(-c3ccc4oc5ccccc5c4c3)ccc12 ; C(#C[Si](c1ccccc1)(c1ccccc1)c1ccc2oc3ccccc3c2c1)c1c2ccccc2c(-c2ccc3ccccc3c2)c2ccccc12 ; C(#C[Si](c1ccccc1)(c1ccccc1)c1ccc2oc3ccccc3c2c1)c1c2ccccc2c(-c2ccccc2)c2cc(-c3ccccc3)ccc12]

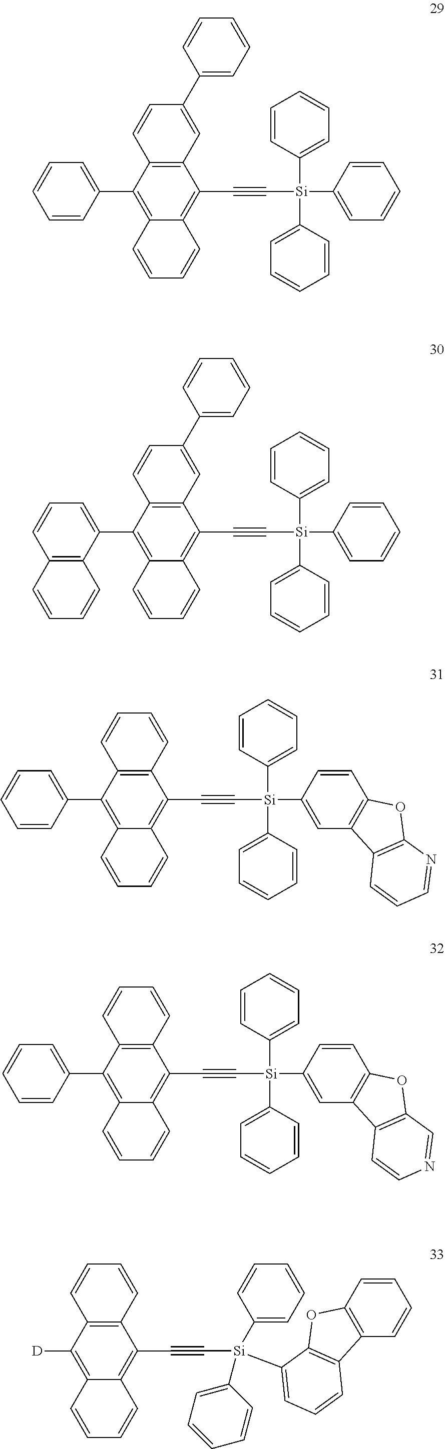 [SMILES: C(#C[Si](c1ccccc1)(c1ccccc1)c1ccccc1)c1c2ccccc2c(-c2ccccc2)c2ccc(-c3ccccc3)cc12 ; C(#C[Si](c1ccccc1)(c1ccccc1)c1ccccc1)c1c2ccccc2c(-c2cccc3ccccc23)c2ccc(-c3ccccc3)cc12 ; C(#C[Si](c1ccccc1)(c1ccccc1)c1ccc2oc3ncccc3c2c1)c1c2ccccc2c(-c2ccccc2)c2ccccc12 ; C(#C[Si](c1ccccc1)(c1ccccc1)c1ccc2oc3cnccc3c2c1)c1c2ccccc2c(-c2ccccc2)c2ccccc12 ; [2H]c1c2ccccc2c(C#C[Si](c2ccccc2)(c2ccccc2)c2cccc3c2oc2ccccc23)c2ccccc12]

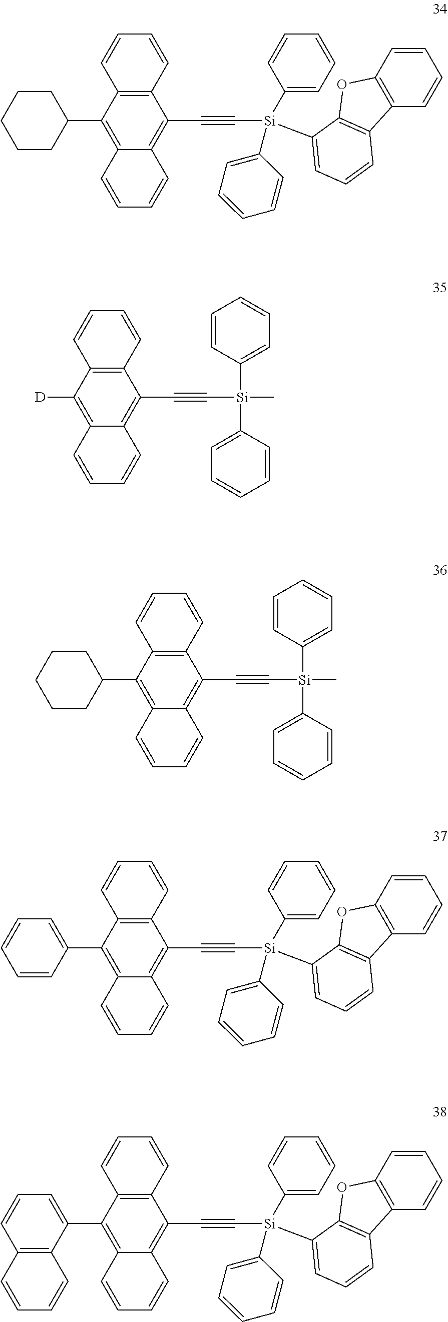 [SMILES: C(#C[Si](c1ccccc1)(c1ccccc1)c1cccc2c1oc1ccccc12)c1c2ccccc2c(C2CCCCC2)c2ccccc12 ; [2H]c1c2ccccc2c(C#C[Si](C)(c2ccccc2)c2ccccc2)c2ccccc12 ; C[Si](C#Cc1c2ccccc2c(C2CCCCC2)c2ccccc12)(c1ccccc1)c1ccccc1 ; C(#C[Si](c1ccccc1)(c1ccccc1)c1cccc2c1oc1ccccc12)c1c2ccccc2c(-c2ccccc2)c2ccccc12 ; C(#C[Si](c1ccccc1)(c1ccccc1)c1cccc2c1oc1ccccc12)c1c2ccccc2c(-c2cccc3ccccc23)c2ccccc12]

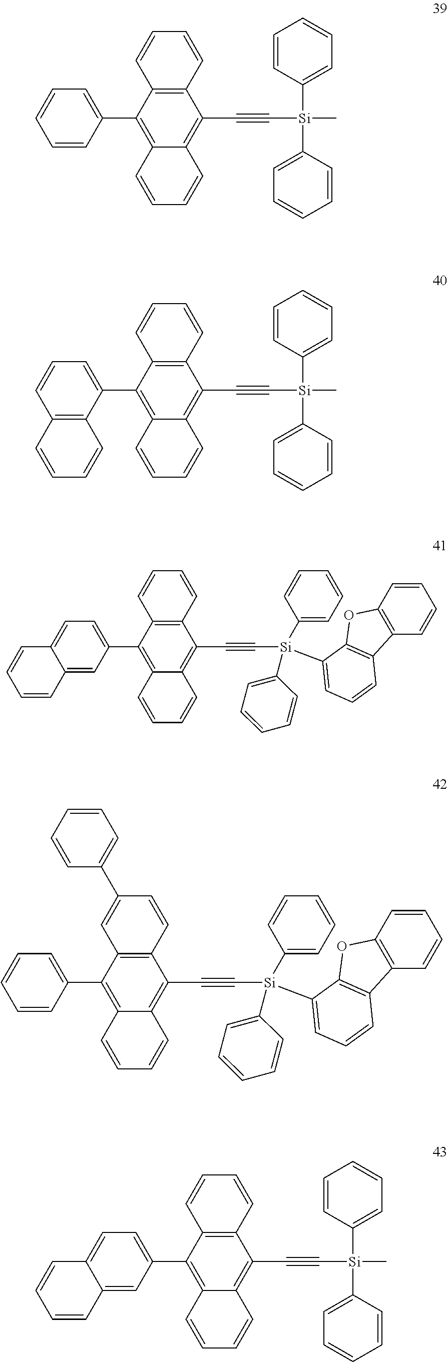 [SMILES: C[Si](C#Cc1c2ccccc2c(-c2ccccc2)c2ccccc12)(c1ccccc1)c1ccccc1 ; C[Si](C#Cc1c2ccccc2c(-c2cccc3ccccc23)c2ccccc12)(c1ccccc1)c1ccccc1 ; C(#C[Si](c1ccccc1)(c1ccccc1)c1cccc2c1oc1ccccc12)c1c2ccccc2c(-c2ccc3ccccc3c2)c2ccccc12 ; C(#C[Si](c1ccccc1)(c1ccccc1)c1cccc2c1oc1ccccc12)c1c2ccccc2c(-c2ccccc2)c2cc(-c3ccccc3)ccc12 ; C[Si](C#Cc1c2ccccc2c(-c2ccc3ccccc3c2)c2ccccc12)(c1ccccc1)c1ccccc1]

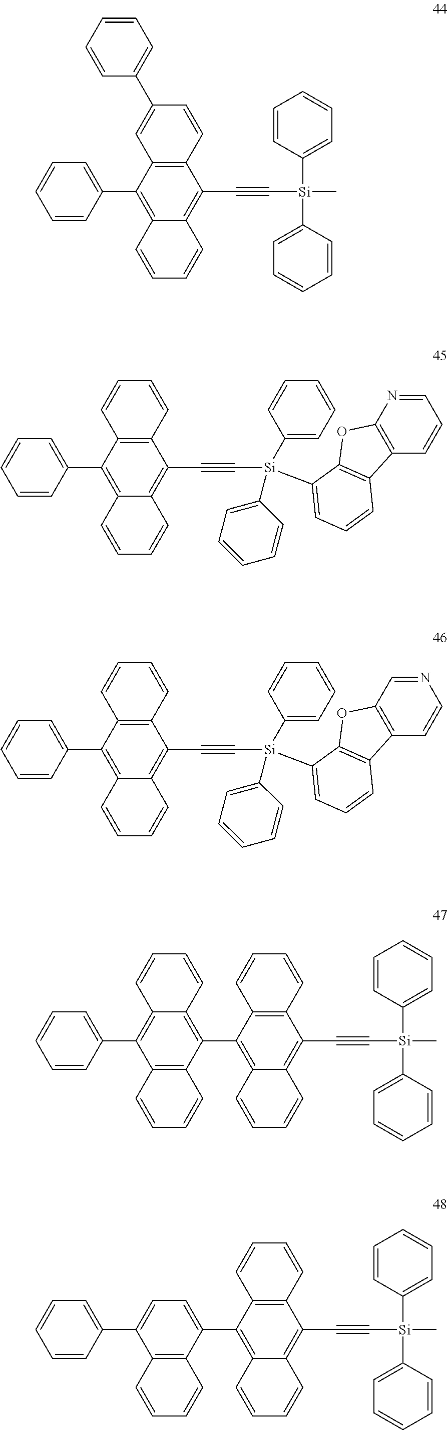 [SMILES: C[Si](C#Cc1c2ccccc2c(-c2ccccc2)c2cc(-c3ccccc3)ccc12)(c1ccccc1)c1ccccc1 ; C(#C[Si](c1ccccc1)(c1ccccc1)c1cccc2c1oc1ncccc12)c1c2ccccc2c(-c2ccccc2)c2ccccc12 ; C(#C[Si](c1ccccc1)(c1ccccc1)c1cccc2c1oc1cnccc12)c1c2ccccc2c(-c2ccccc2)c2ccccc12 ; C[Si](C#Cc1c2ccccc2c(-c2c3ccccc3c(-c3ccccc3)c3ccccc23)c2ccccc12)(c1ccccc1)c1ccccc1 ; C[Si](C#Cc1c2ccccc2c(-c2ccc(-c3ccccc3)c3ccccc23)c2ccccc12)(c1ccccc1)c1ccccc1]

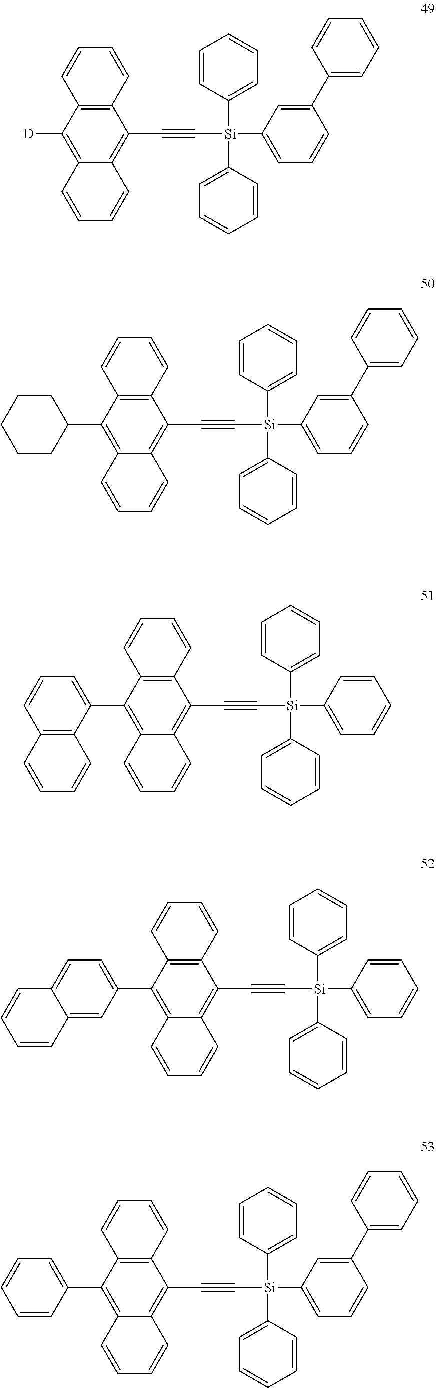 [SMILES: [2H]c1c2ccccc2c(C#C[Si](c2ccccc2)(c2ccccc2)c2cccc(-c3ccccc3)c2)c2ccccc12 ; C(#C[Si](c1ccccc1)(c1ccccc1)c1cccc(-c2ccccc2)c1)c1c2ccccc2c(C2CCCCC2)c2ccccc12 ; C(#C[Si](c1ccccc1)(c1ccccc1)c1ccccc1)c1c2ccccc2c(-c2cccc3ccccc23)c2ccccc12 ; C(#C[Si](c1ccccc1)(c1ccccc1)c1ccccc1)c1c2ccccc2c(-c2ccc3ccccc3c2)c2ccccc12 ; C(#C[Si](c1ccccc1)(c1ccccc1)c1cccc(-c2ccccc2)c1)c1c2ccccc2c(-c2ccccc2)c2ccccc12]

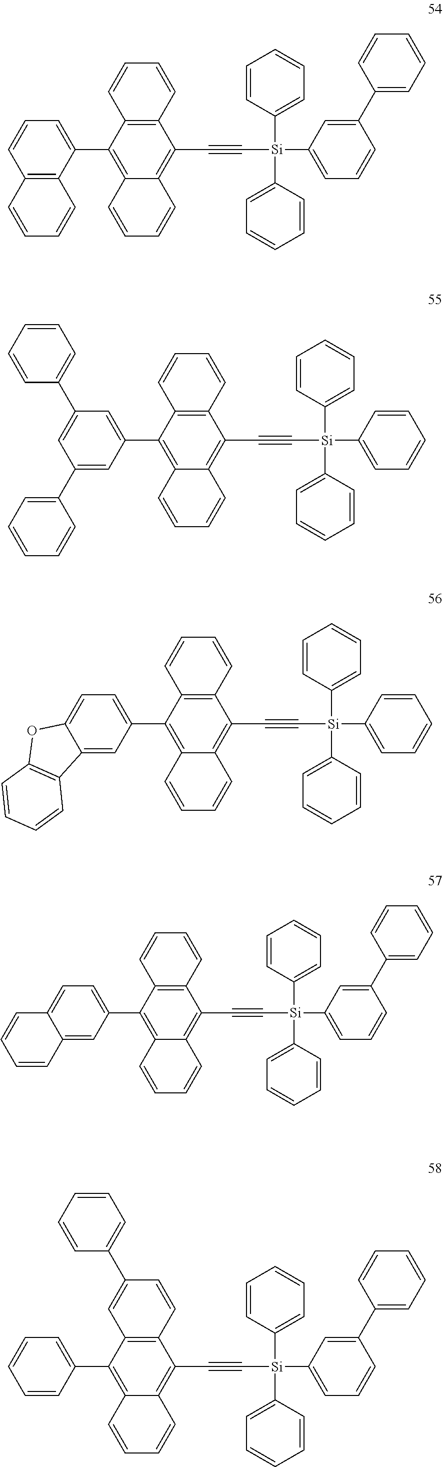 [SMILES: C(#C[Si](c1ccccc1)(c1ccccc1)c1cccc(-c2ccccc2)c1)c1c2ccccc2c(-c2cccc3ccccc23)c2ccccc12 ; C(#C[Si](c1ccccc1)(c1ccccc1)c1ccccc1)c1c2ccccc2c(-c2cc(-c3ccccc3)cc(-c3ccccc3)c2)c2ccccc12 ; C(#C[Si](c1ccccc1)(c1ccccc1)c1ccccc1)c1c2ccccc2c(-c2ccc3oc4ccccc4c3c2)c2ccccc12 ; C(#C[Si](c1ccccc1)(c1ccccc1)c1cccc(-c2ccccc2)c1)c1c2ccccc2c(-c2ccc3ccccc3c2)c2ccccc12 ; C(#C[Si](c1ccccc1)(c1ccccc1)c1cccc(-c2ccccc2)c1)c1c2ccccc2c(-c2ccccc2)c2cc(-c3ccccc3)ccc12]

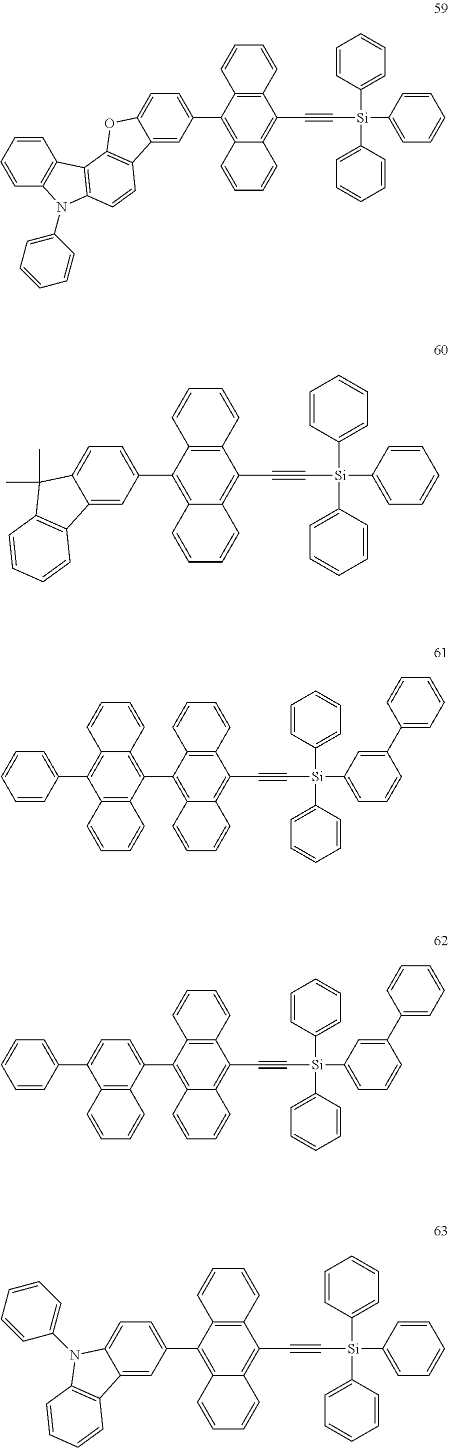 [SMILES: C(#C[Si](c1ccccc1)(c1ccccc1)c1ccccc1)c1c2ccccc2c(-c2ccc3oc4c(ccc5c4c4ccccc4n5-c4ccccc4)c3c2)c2ccccc12 ; CC1(C)c2ccccc2-c2cc(-c3c4ccccc4c(C#C[Si](c4ccccc4)(c4ccccc4)c4ccccc4)c4ccccc34)ccc21 ; C(#C[Si](c1ccccc1)(c1ccccc1)c1cccc(-c2ccccc2)c1)c1c2ccccc2c(-c2c3ccccc3c(-c3ccccc3)c3ccccc23)c2ccccc12 ; C(#C[Si](c1ccccc1)(c1ccccc1)c1cccc(-c2ccccc2)c1)c1c2ccccc2c(-c2ccc(-c3ccccc3)c3ccccc23)c2ccccc12 ; C(#C[Si](c1ccccc1)(c1ccccc1)c1ccccc1)c1c2ccccc2c(-c2ccc3c(c2)c2ccccc2n3-c2ccccc2)c2ccccc12]

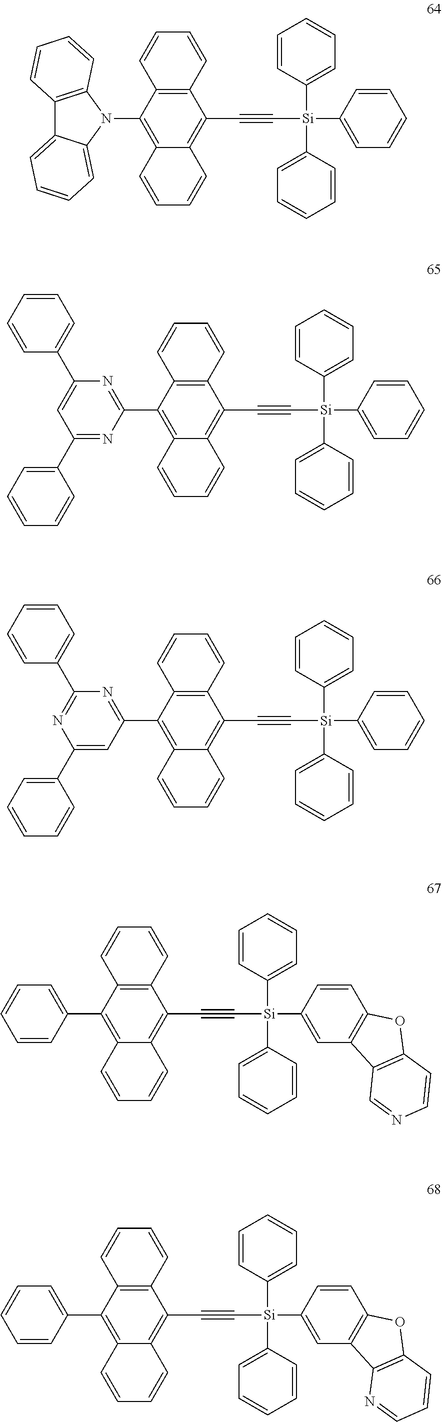 [SMILES: C(#C[Si](c1ccccc1)(c1ccccc1)c1ccccc1)c1c2ccccc2c(-n2c3ccccc3c3ccccc32)c2ccccc12 ; C(#C[Si](c1ccccc1)(c1ccccc1)c1ccccc1)c1c2ccccc2c(-c2nc(-c3ccccc3)cc(-c3ccccc3)n2)c2ccccc12 ; C(#C[Si](c1ccccc1)(c1ccccc1)c1ccccc1)c1c2ccccc2c(-c2cc(-c3ccccc3)nc(-c3ccccc3)n2)c2ccccc12 ; C(#C[Si](c1ccccc1)(c1ccccc1)c1ccc2oc3ccncc3c2c1)c1c2ccccc2c(-c2ccccc2)c2ccccc12 ; C(#C[Si](c1ccccc1)(c1ccccc1)c1ccc2oc3cccnc3c2c1)c1c2ccccc2c(-c2ccccc2)c2ccccc12]

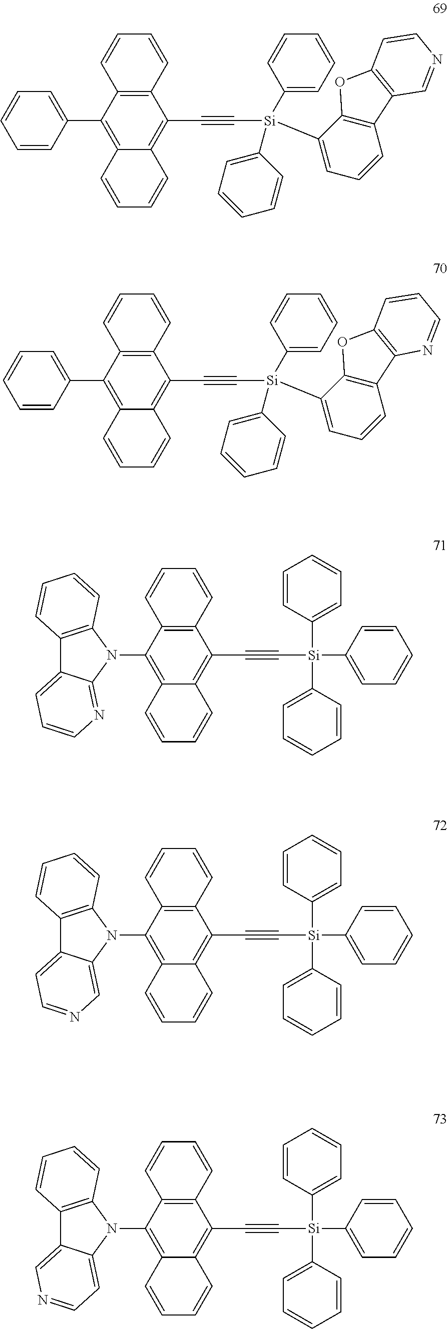 [SMILES: C(#C[Si](c1ccccc1)(c1ccccc1)c1cccc2c1oc1ccncc12)c1c2ccccc2c(-c2ccccc2)c2ccccc12 ; C(#C[Si](c1ccccc1)(c1ccccc1)c1cccc2c1oc1cccnc12)c1c2ccccc2c(-c2ccccc2)c2ccccc12 ; C(#C[Si](c1ccccc1)(c1ccccc1)c1ccccc1)c1c2ccccc2c(-n2c3ccccc3c3cccnc32)c2ccccc12 ; C(#C[Si](c1ccccc1)(c1ccccc1)c1ccccc1)c1c2ccccc2c(-n2c3ccccc3c3ccncc32)c2ccccc12 ; C(#C[Si](c1ccccc1)(c1ccccc1)c1ccccc1)c1c2ccccc2c(-n2c3ccccc3c3cnccc32)c2ccccc12]

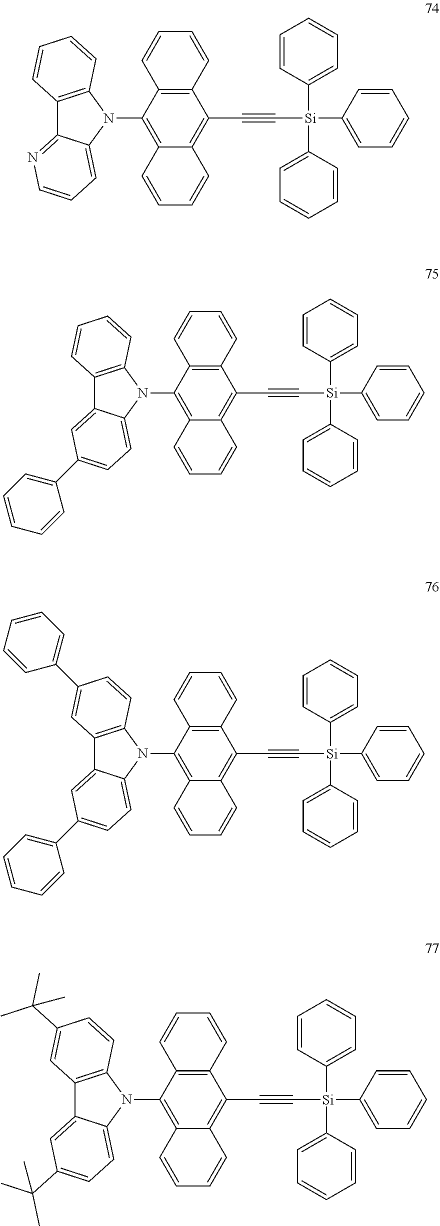 [SMILES: C(#C[Si](c1ccccc1)(c1ccccc1)c1ccccc1)c1c2ccccc2c(-n2c3ccccc3c3ncccc32)c2ccccc12 ; C(#C[Si](c1ccccc1)(c1ccccc1)c1ccccc1)c1c2ccccc2c(-n2c3ccccc3c3cc(-c4ccccc4)ccc32)c2ccccc12 ; C(#C[Si](c1ccccc1)(c1ccccc1)c1ccccc1)c1c2ccccc2c(-n2c3ccc(-c4ccccc4)cc3c3cc(-c4ccccc4)ccc32)c2ccccc12 ; CC(C)(C)c1ccc2c(c1)c1cc(C(C)(C)C)ccc1n2-c1c2ccccc2c(C#C[Si](c2ccccc2)(c2ccccc2)c2ccccc2)c2ccccc12]

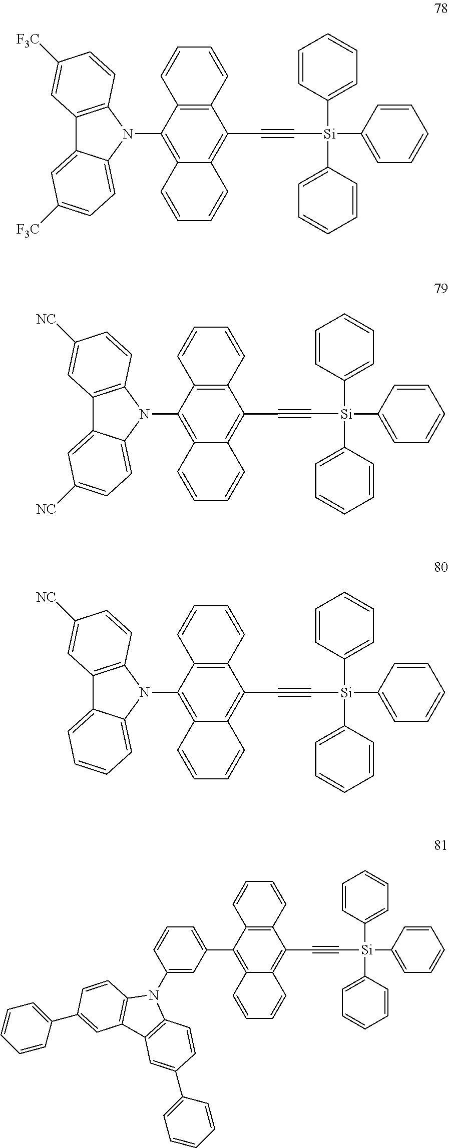 [SMILES: FC(F)(F)c1ccc2c(c1)c1cc(C(F)(F)F)ccc1n2-c1c2ccccc2c(C#C[Si](c2ccccc2)(c2ccccc2)c2ccccc2)c2ccccc12 ; N#Cc1ccc2c(c1)c1cc(C#N)ccc1n2-c1c2ccccc2c(C#C[Si](c2ccccc2)(c2ccccc2)c2ccccc2)c2ccccc12 ; N#Cc1ccc2c(c1)c1ccccc1n2-c1c2ccccc2c(C#C[Si](c2ccccc2)(c2ccccc2)c2ccccc2)c2ccccc12 ; C(#C[Si](c1ccccc1)(c1ccccc1)c1ccccc1)c1c2ccccc2c(-c2cccc(-n3c4ccc(-c5ccccc5)cc4c4cc(-c5ccccc5)ccc43)c2)c2ccccc12]

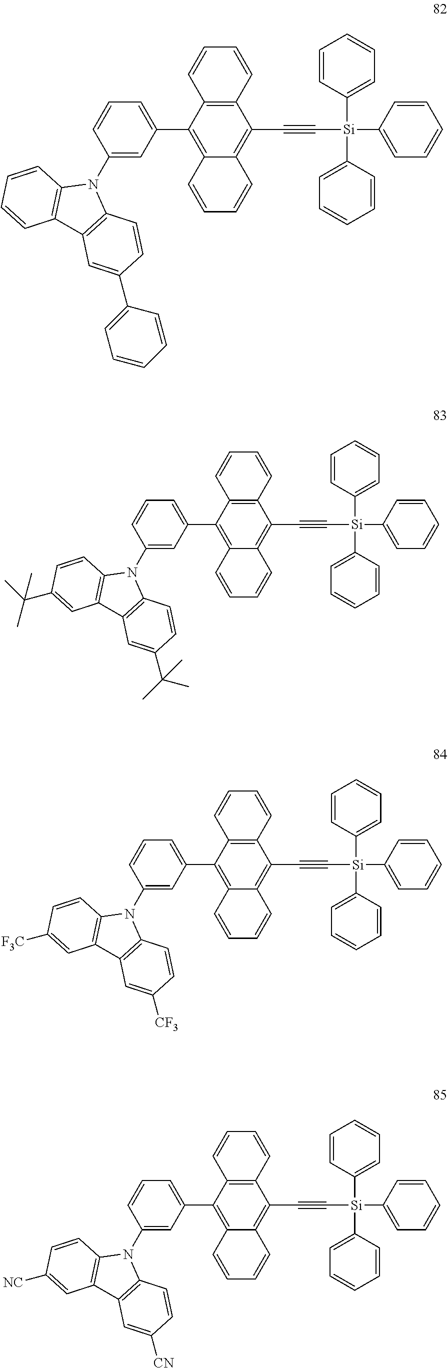 [SMILES: C(#C[Si](c1ccccc1)(c1ccccc1)c1ccccc1)c1c2ccccc2c(-c2cccc(-n3c4ccccc4c4cc(-c5ccccc5)ccc43)c2)c2ccccc12 ; CC(C)(C)c1ccc2c(c1)c1cc(C(C)(C)C)ccc1n2-c1cccc(-c2c3ccccc3c(C#C[Si](c3ccccc3)(c3ccccc3)c3ccccc3)c3ccccc23)c1 ; FC(F)(F)c1ccc2c(c1)c1cc(C(F)(F)F)ccc1n2-c1cccc(-c2c3ccccc3c(C#C[Si](c3ccccc3)(c3ccccc3)c3ccccc3)c3ccccc23)c1 ; N#Cc1ccc2c(c1)c1cc(C#N)ccc1n2-c1cccc(-c2c3ccccc3c(C#C[Si](c3ccccc3)(c3ccccc3)c3ccccc3)c3ccccc23)c1]

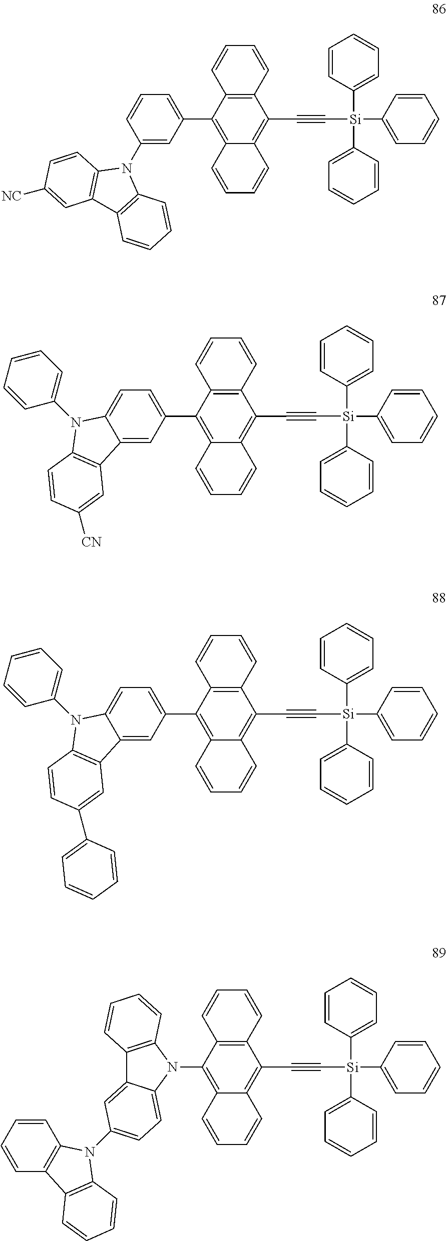 [SMILES: N#Cc1ccc2c(c1)c1ccccc1n2-c1cccc(-c2c3ccccc3c(C#C[Si](c3ccccc3)(c3ccccc3)c3ccccc3)c3ccccc23)c1 ; N#Cc1ccc2c(c1)c1cc(-c3c4ccccc4c(C#C[Si](c4ccccc4)(c4ccccc4)c4ccccc4)c4ccccc34)ccc1n2-c1ccccc1 ; C(#C[Si](c1ccccc1)(c1ccccc1)c1ccccc1)c1c2ccccc2c(-c2ccc3c(c2)c2cc(-c4ccccc4)ccc2n3-c2ccccc2)c2ccccc12 ; C(#C[Si](c1ccccc1)(c1ccccc1)c1ccccc1)c1c2ccccc2c(-n2c3ccccc3c3cc(-n4c5ccccc5c5ccccc54)ccc32)c2ccccc12]

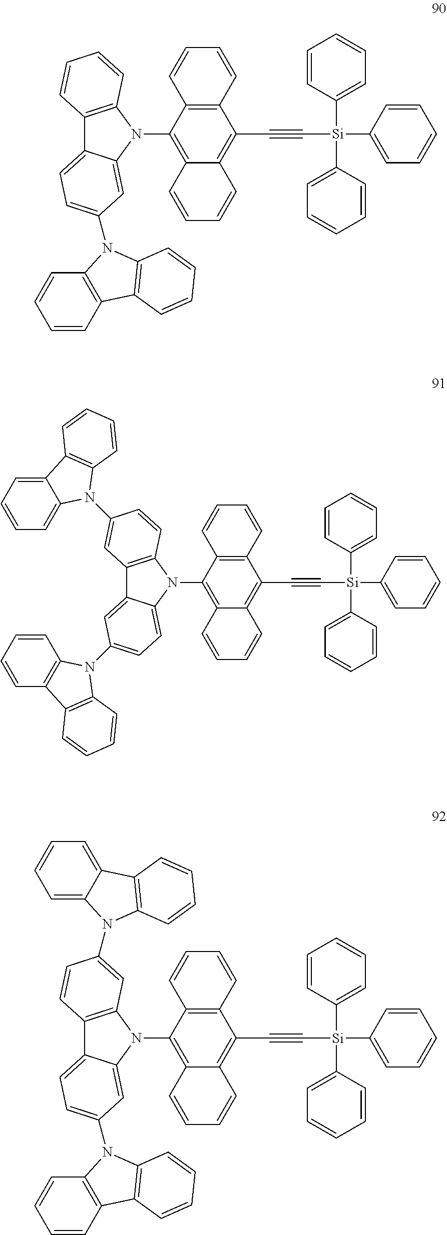 [SMILES: C(#C[Si](c1ccccc1)(c1ccccc1)c1ccccc1)c1c2ccccc2c(-n2c3ccccc3c3ccc(-n4c5ccccc5c5ccccc54)cc32)c2ccccc12 ; C(#C[Si](c1ccccc1)(c1ccccc1)c1ccccc1)c1c2ccccc2c(-n2c3ccc(-n4c5ccccc5c5ccccc54)cc3c3cc(-n4c5ccccc5c5ccccc54)ccc32)c2ccccc12 ; C(#C[Si](c1ccccc1)(c1ccccc1)c1ccccc1)c1c2ccccc2c(-n2c3cc(-n4c5ccccc5c5ccccc54)ccc3c3ccc(-n4c5ccccc5c5ccccc54)cc32)c2ccccc12]

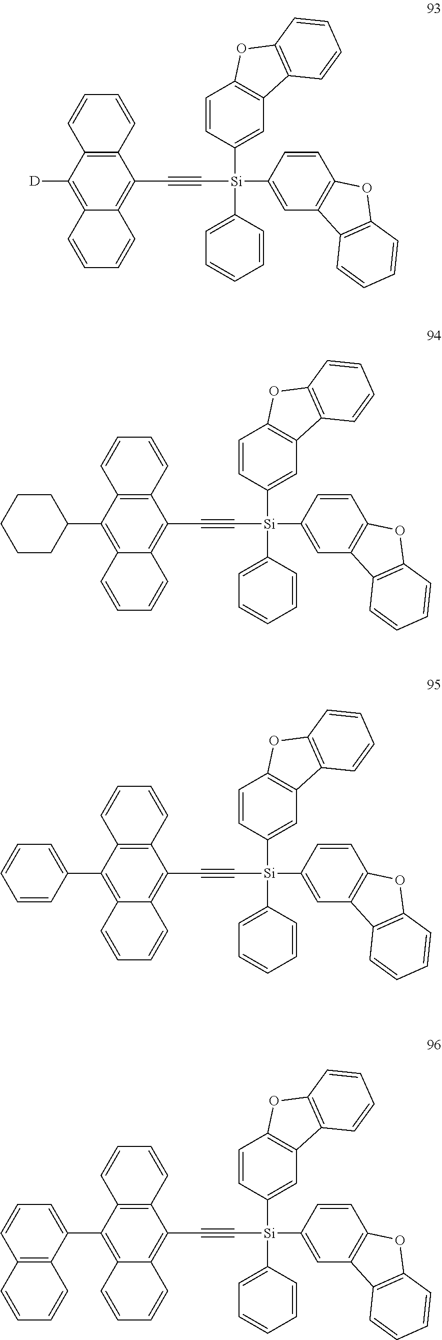 [SMILES: [2H]c1c2ccccc2c(C#C[Si](c2ccccc2)(c2ccc3oc4ccccc4c3c2)c2ccc3oc4ccccc4c3c2)c2ccccc12 ; C(#C[Si](c1ccccc1)(c1ccc2oc3ccccc3c2c1)c1ccc2oc3ccccc3c2c1)c1c2ccccc2c(C2CCCCC2)c2ccccc12 ; C(#C[Si](c1ccccc1)(c1ccc2oc3ccccc3c2c1)c1ccc2oc3ccccc3c2c1)c1c2ccccc2c(-c2ccccc2)c2ccccc12 ; C(#C[Si](c1ccccc1)(c1ccc2oc3ccccc3c2c1)c1ccc2oc3ccccc3c2c1)c1c2ccccc2c(-c2cccc3ccccc23)c2ccccc12]

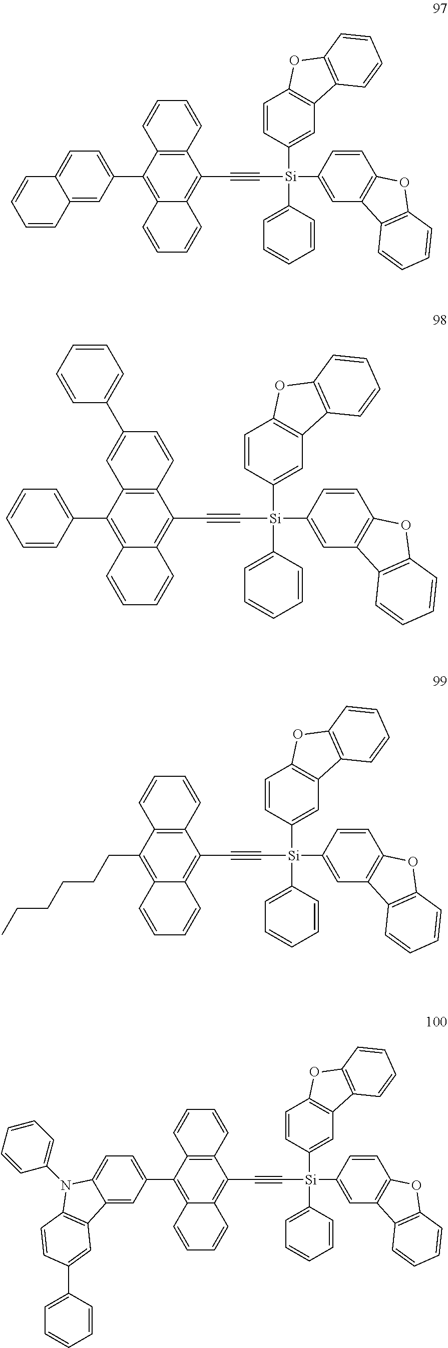 [SMILES: C(#C[Si](c1ccccc1)(c1ccc2oc3ccccc3c2c1)c1ccc2oc3ccccc3c2c1)c1c2ccccc2c(-c2ccc3ccccc3c2)c2ccccc12 ; C(#C[Si](c1ccccc1)(c1ccc2oc3ccccc3c2c1)c1ccc2oc3ccccc3c2c1)c1c2ccccc2c(-c2ccccc2)c2cc(-c3ccccc3)ccc12 ; CCCCCCc1c2ccccc2c(C#C[Si](c2ccccc2)(c2ccc3oc4ccccc4c3c2)c2ccc3oc4ccccc4c3c2)c2ccccc12 ; C(#C[Si](c1ccccc1)(c1ccc2oc3ccccc3c2c1)c1ccc2oc3ccccc3c2c1)c1c2ccccc2c(-c2ccc3c(c2)c2cc(-c4ccccc4)ccc2n3-c2ccccc2)c2ccccc12]

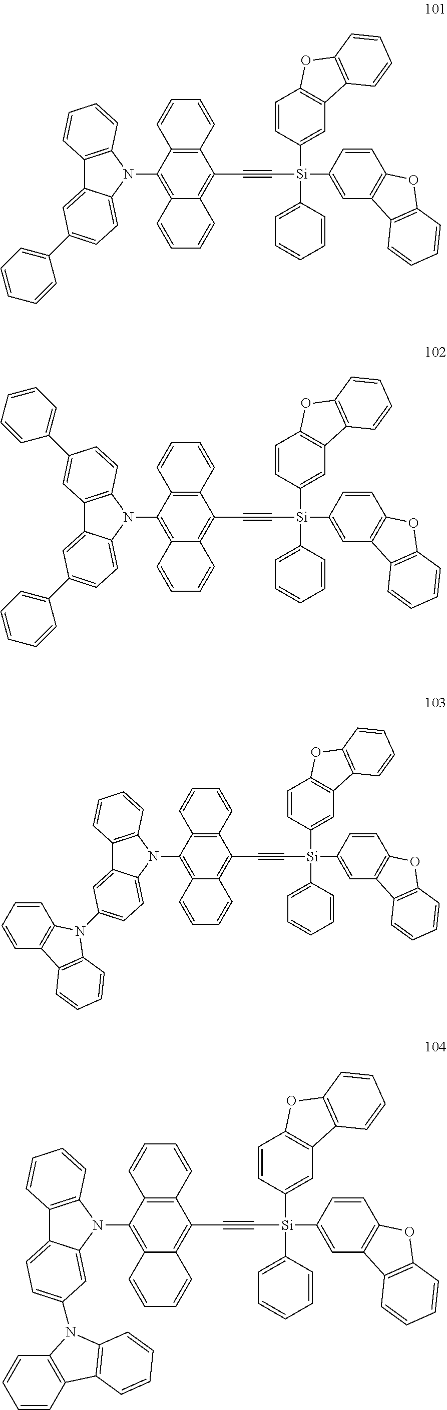 [SMILES: C(#C[Si](c1ccccc1)(c1ccc2oc3ccccc3c2c1)c1ccc2oc3ccccc3c2c1)c1c2ccccc2c(-n2c3ccccc3c3cc(-c4ccccc4)ccc32)c2ccccc12 ; C(#C[Si](c1ccccc1)(c1ccc2oc3ccccc3c2c1)c1ccc2oc3ccccc3c2c1)c1c2ccccc2c(-n2c3ccc(-c4ccccc4)cc3c3cc(-c4ccccc4)ccc32)c2ccccc12 ; C(#C[Si](c1ccccc1)(c1ccc2oc3ccccc3c2c1)c1ccc2oc3ccccc3c2c1)c1c2ccccc2c(-n2c3ccccc3c3cc(-n4c5ccccc5c5ccccc54)ccc32)c2ccccc12 ; C(#C[Si](c1ccccc1)(c1ccc2oc3ccccc3c2c1)c1ccc2oc3ccccc3c2c1)c1c2ccccc2c(-n2c3ccccc3c3ccc(-n4c5ccccc5c5ccccc54)cc32)c2ccccc12]

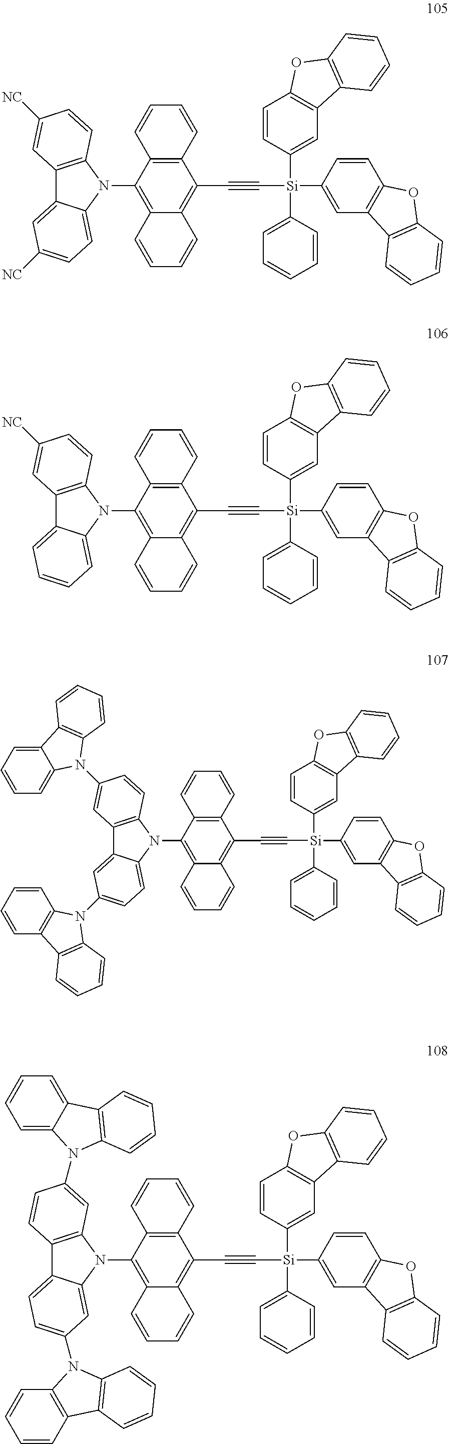

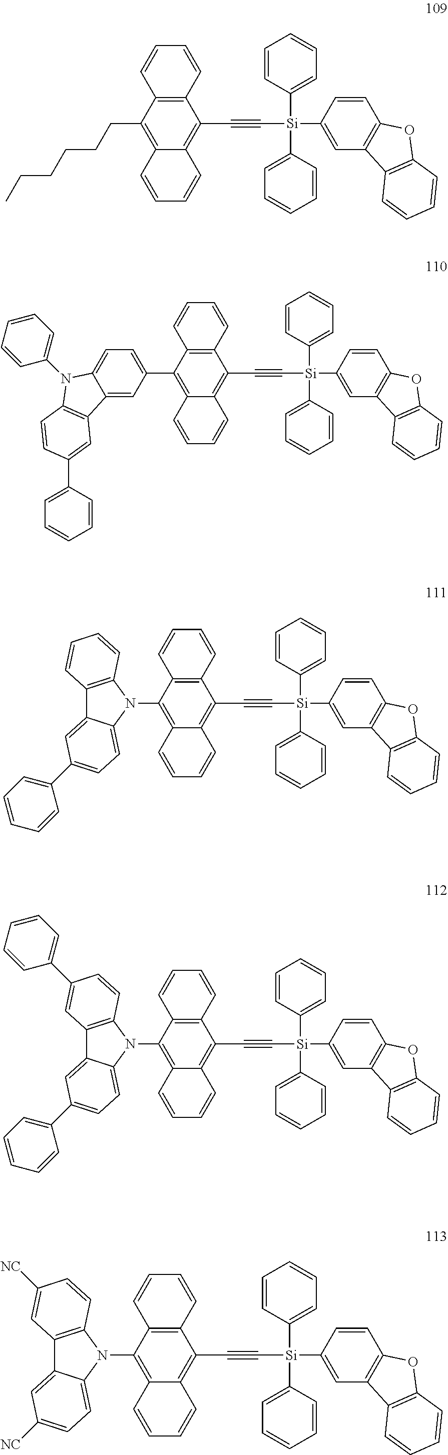

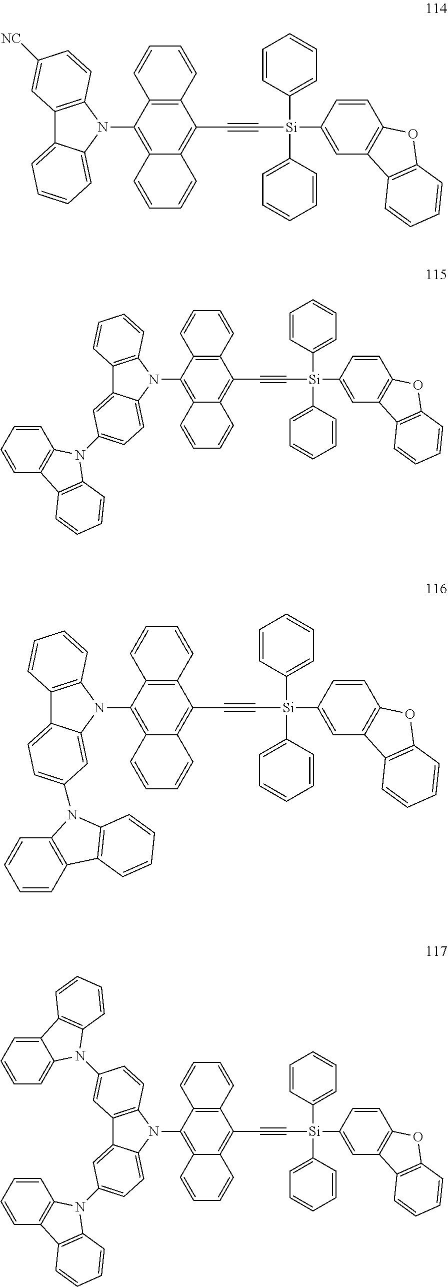 [SMILES: N#Cc1ccc2c(c1)c1ccccc1n2-c1c2ccccc2c(C#C[Si](c2ccccc2)(c2ccccc2)c2ccc3oc4ccccc4c3c2)c2ccccc12 ; C(#C[Si](c1ccccc1)(c1ccccc1)c1ccc2oc3ccccc3c2c1)c1c2ccccc2c(-n2c3ccccc3c3cc(-n4c5ccccc5c5ccccc54)ccc32)c2ccccc12 ; C(#C[Si](c1ccccc1)(c1ccccc1)c1ccc2oc3ccccc3c2c1)c1c2ccccc2c(-n2c3ccccc3c3ccc(-n4c5ccccc5c5ccccc54)cc32)c2ccccc12 ; C(#C[Si](c1ccccc1)(c1ccccc1)c1ccc2oc3ccccc3c2c1)c1c2ccccc2c(-n2c3ccc(-n4c5ccccc5c5ccccc54)cc3c3cc(-n4c5ccccc5c5ccccc54)ccc32)c2ccccc12]

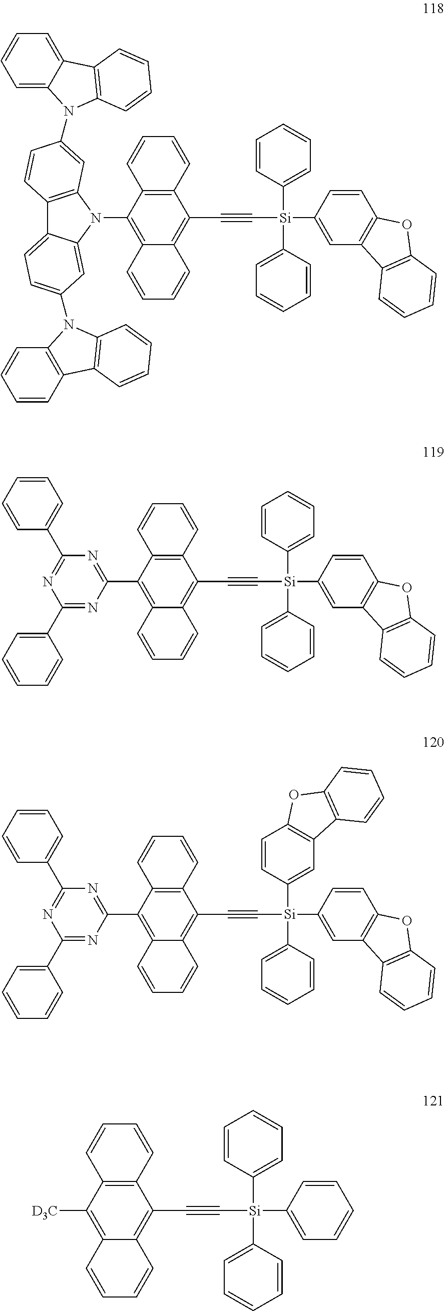

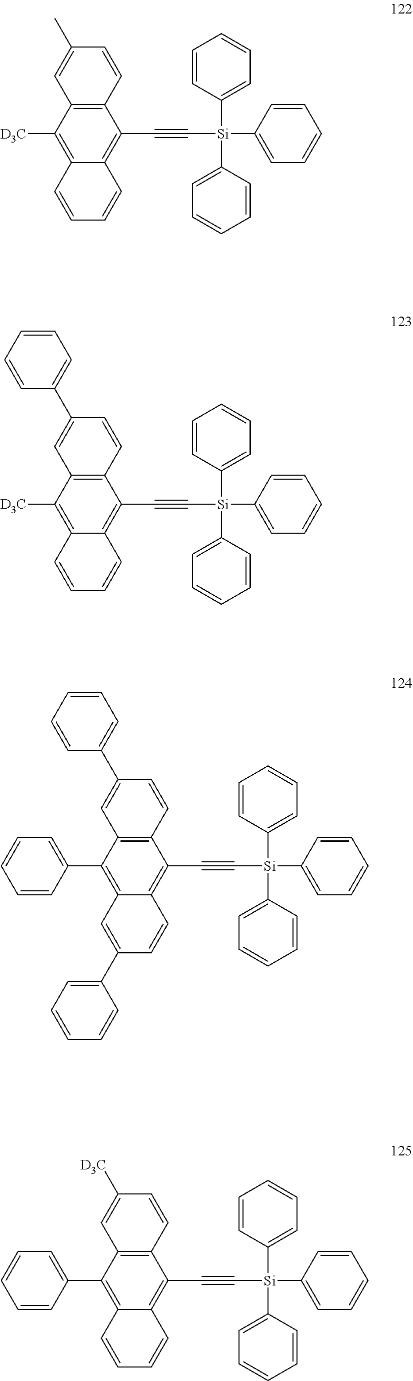

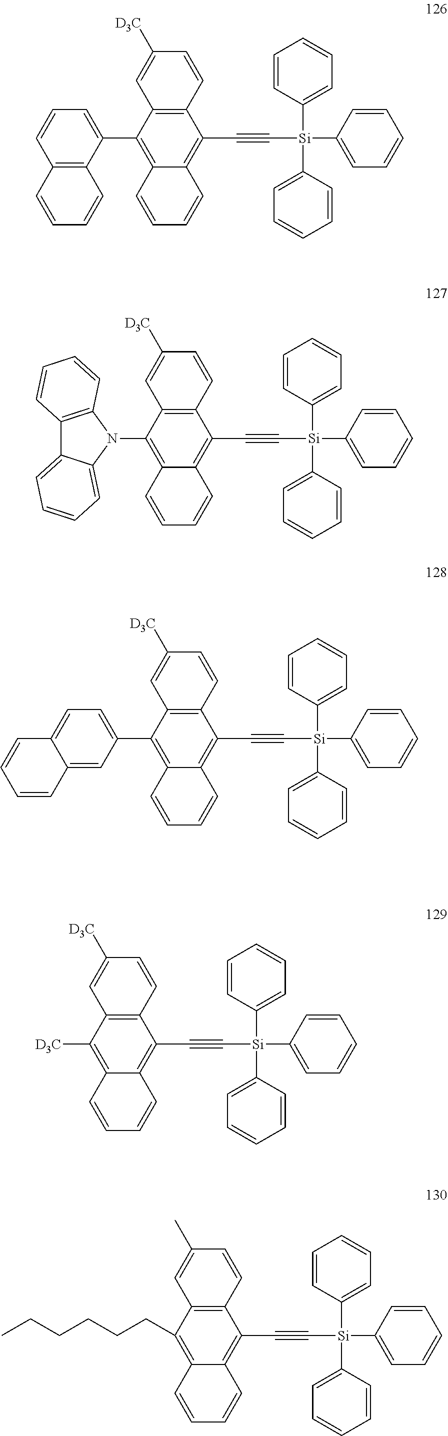

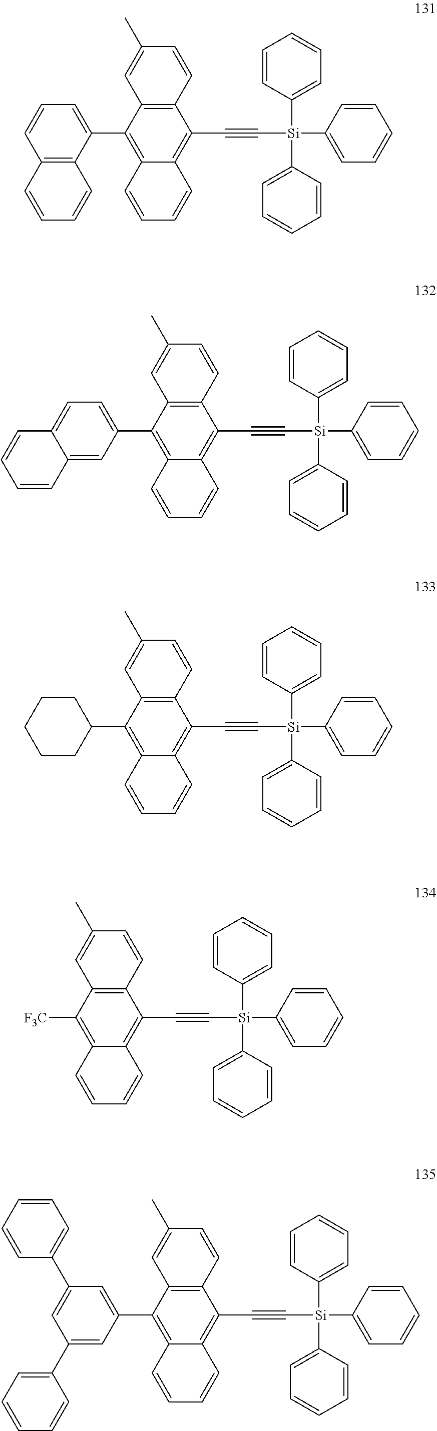

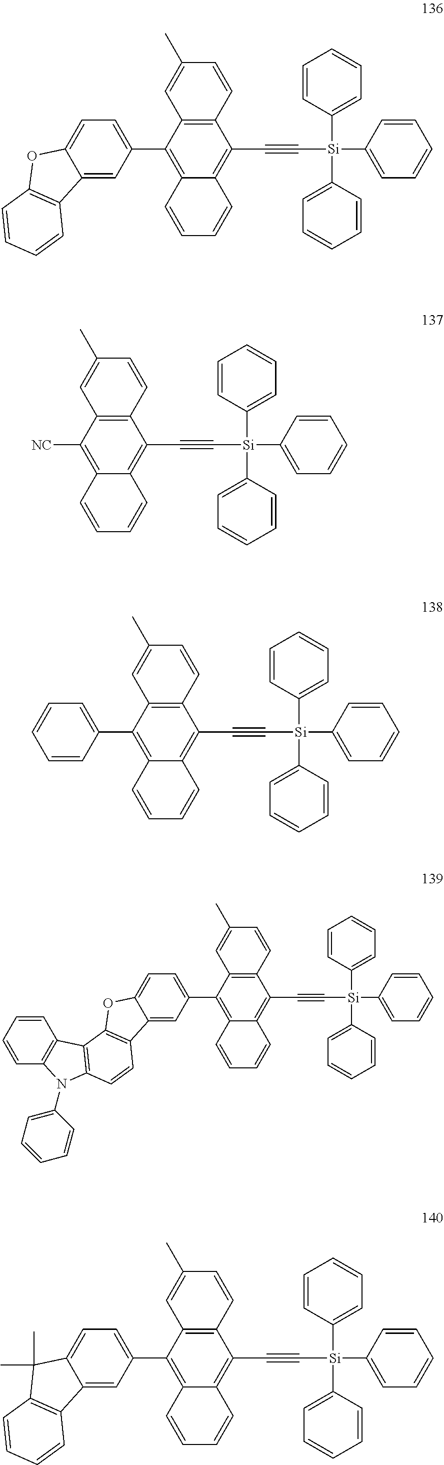

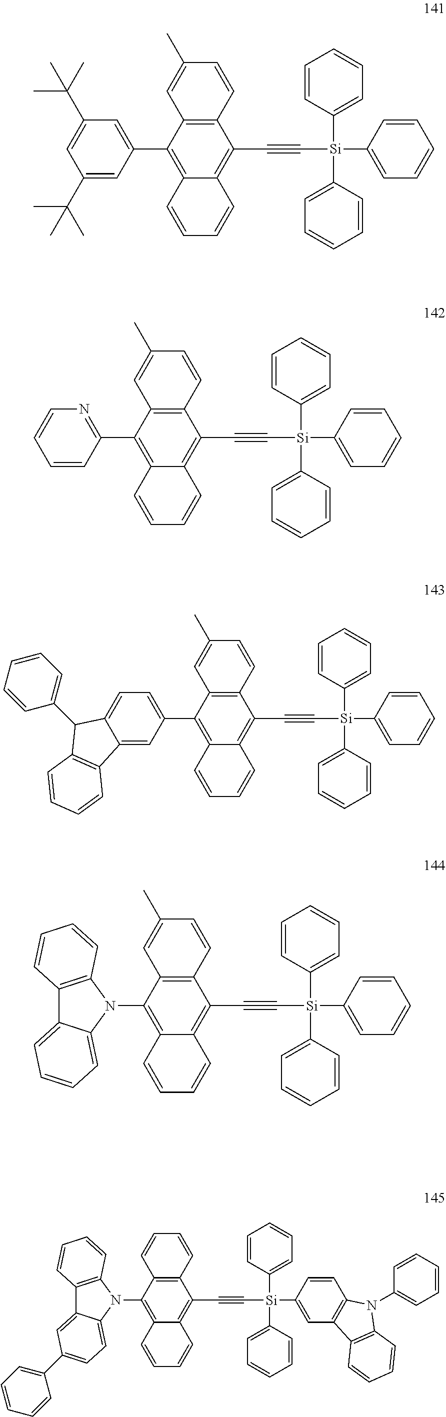

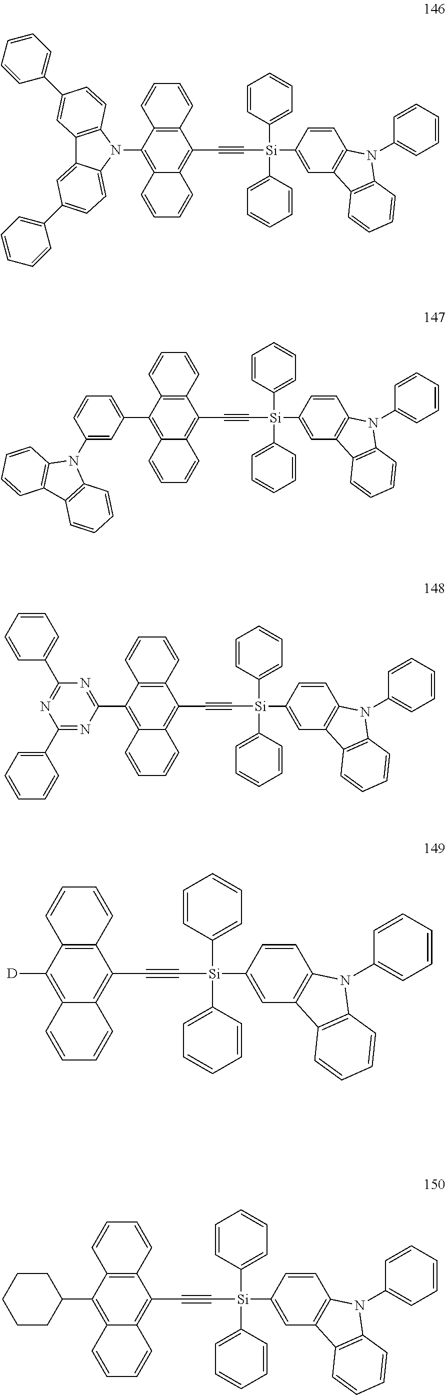

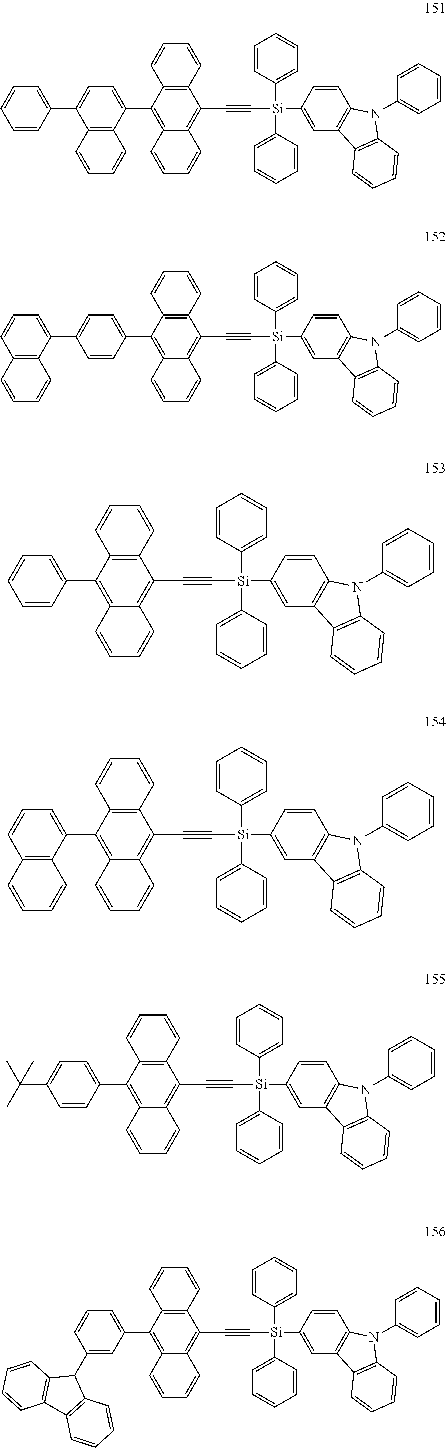

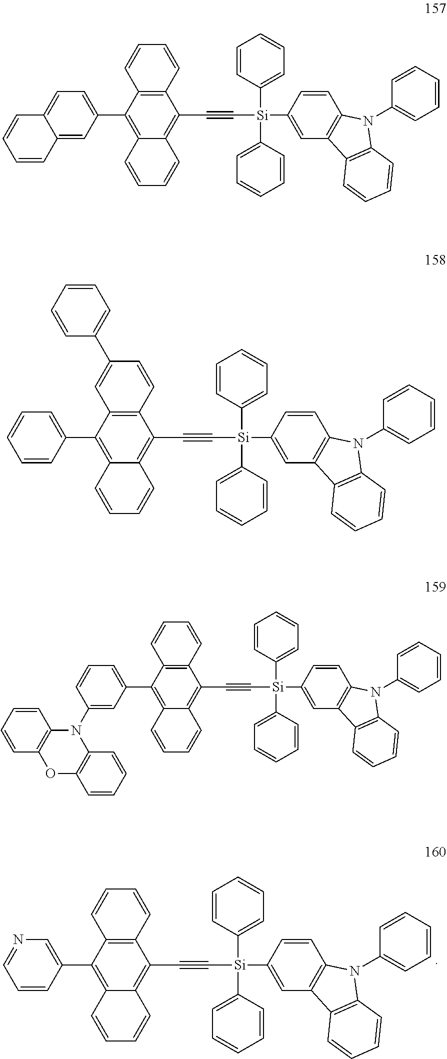 [SMILES: C(#C[Si](c1ccccc1)(c1ccccc1)c1ccc2c(c1)c1ccccc1n2-c1ccccc1)c1c2ccccc2c(-c2ccc3ccccc3c2)c2ccccc12 ; C(#C[Si](c1ccccc1)(c1ccccc1)c1ccc2c(c1)c1ccccc1n2-c1ccccc1)c1c2ccccc2c(-c2ccccc2)c2cc(-c3ccccc3)ccc12 ; C(#C[Si](c1ccccc1)(c1ccccc1)c1ccc2c(c1)c1ccccc1n2-c1ccccc1)c1c2ccccc2c(-c2cccc(N3c4ccccc4Oc4ccccc43)c2)c2ccccc12 ; C(#C[Si](c1ccccc1)(c1ccccc1)c1ccc2c(c1)c1ccccc1n2-c1ccccc1)c1c2ccccc2c(-c2cccnc2)c2ccccc12]

In some embodiments, the fluorescent host may be represented by Formula FH-2:

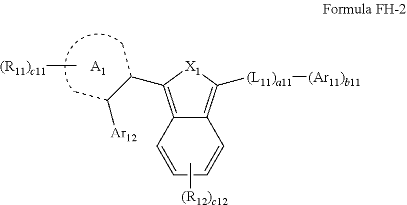

wherein, in Formula FH-2,

X₁ may be O or S,

A₁ may be a C₅-C₆₀ carbocyclic group or a C₁-C₆₀ heterocyclic group,

L₁₁ may be a substituted or unsubstituted C₅-C₆₀ carbocyclic group or a substituted or unsubstituted C₁-C₆₀ heterocyclic group,

a11 may be an integer from 0 to 3,

Ar₁₁ and Ar₁₂ may each independently be a C₆-C₆₀ aryl group, a C₁-C₆₀ heteroaryl group, a monovalent non-aromatic condensed polycyclic group, or a monovalent non-aromatic condensed heteropolycyclic group, each unsubstituted or substituted with at least one Ra,

b11 may be an integer from 1 to 5,

R₁₁, R₁₂, and Ra may each independently be hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent non-aromatic condensed polycyclic group, a substituted or unsubstituted monovalent non-aromatic condensed heteropolycyclic group, —Si(Q₁)(Q₂)(Q₃), —N(Q₄)(Q₅), or —B(Q₆)(Q₇),

c11 may be an integer from 1 to 20,

c12 may be an integer from 1 to 4,

when c11 is 2 or greater, two adjacent R₁₁(s) may optionally be bound to each other to form a substituted or unsubstituted C₅-C₃₀ carbocyclic group or a substituted or unsubstituted C₁-C₃₀ heterocyclic group,

when c12 is 2 or greater, two adjacent R₁₂(s) may optionally be bound to each other to form a substituted or unsubstituted C₅-C₃₀ carbocyclic group or a substituted or unsubstituted C₁-C₃₀ heterocyclic group,

A₁ and Ar₁₂ may optionally be bound to each other via a first linking group a single bond, *—Ar₃₁—*′, *—O—*′, *—S—*′, *—[C(R₃₁)(R₃₂)]k₁₁—*′, *—C(R₃₁)=*′, *═C(R₃₁)—*′, *—C(R₃₁)═C(R₃₂)—*′ *—C(═O)—*′, *—C(═S)—*′, *—C—C—*′, *—N(R₃₁)—*′, *—P(R₃₁)—*′, *—[Si(R₃₁)(R₃₂)]k₁₁—*′, and *—P(R₃₁)(R₃₂)—*′ to form a substituted or unsubstituted C₅-C₃₀ carbocyclic group or a substituted or unsubstituted C₁-C₃₀ heterocyclic group,

Ar₃₁ may be a C₅-C₃₀ carbocyclic group,

R₃₁ and R₃₂ may each be understood by referring to the description of R₁₁ provided herein, and

k₁₁ may be 1,2,3, or 4.

In some embodiments, the fluorescent host represented by Formula FH-2 may be Group FH2, but embodiments are not limited thereto:

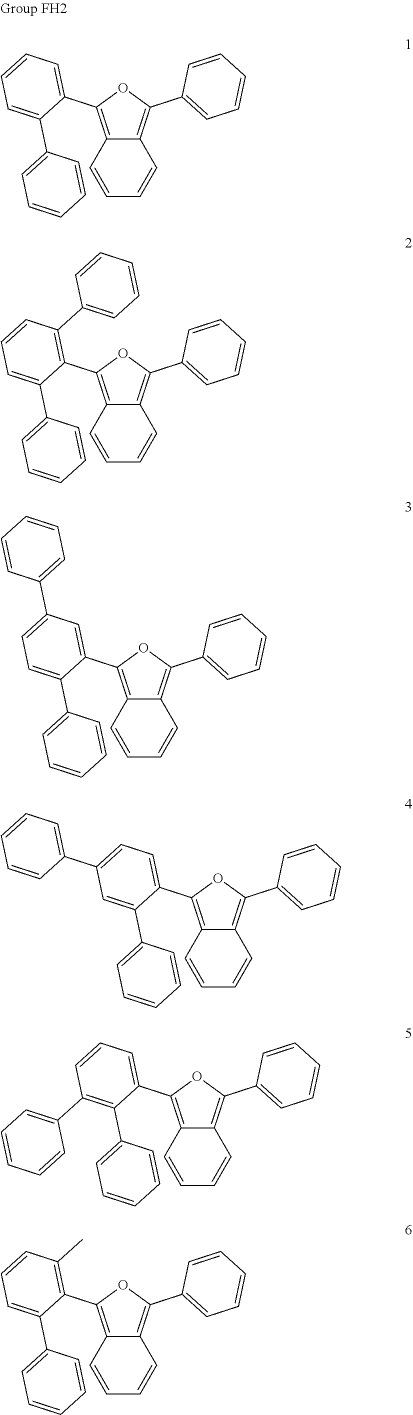

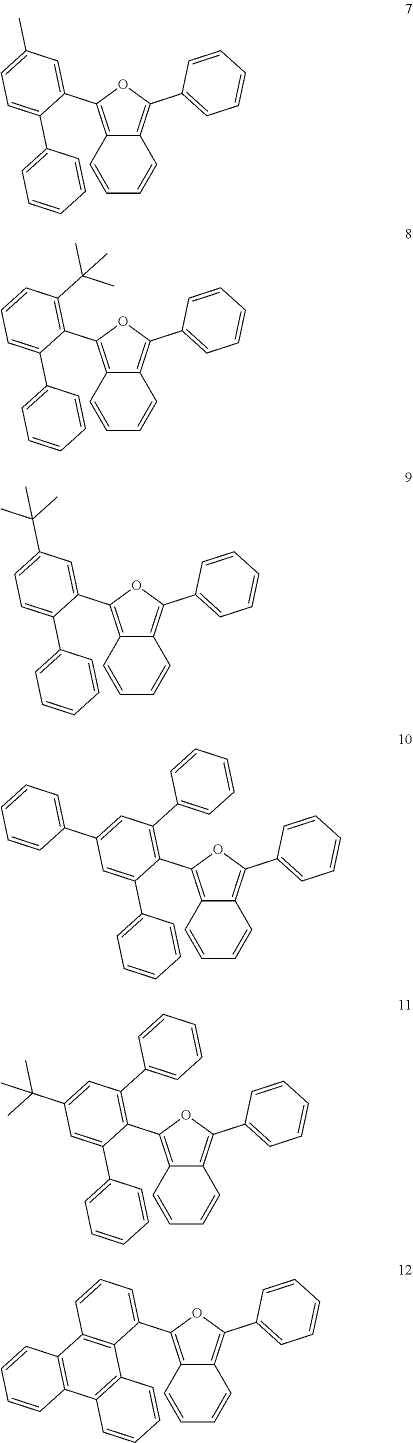

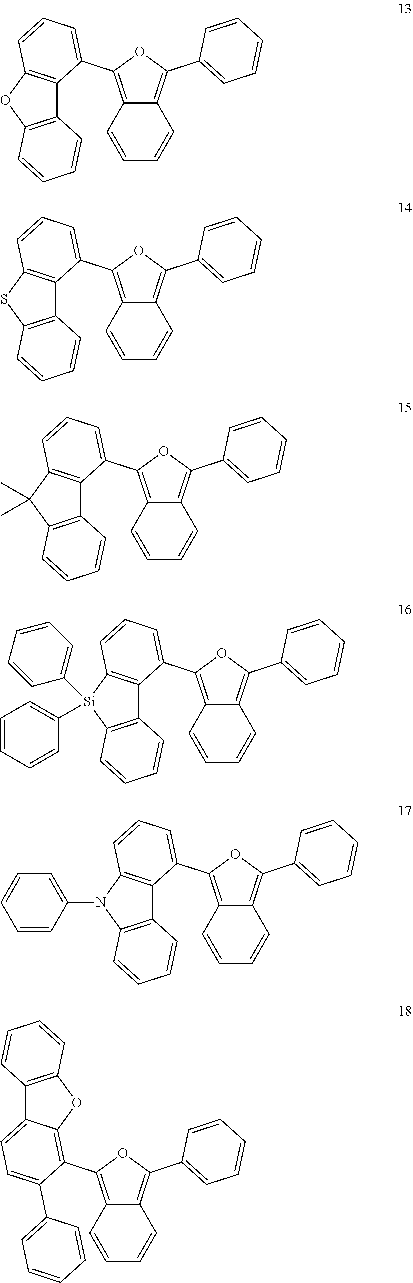

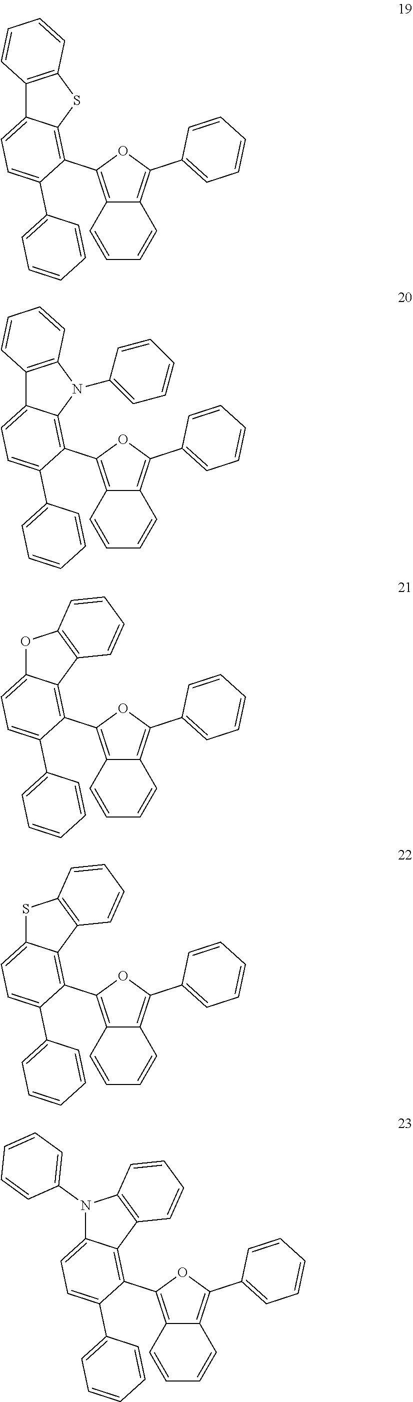

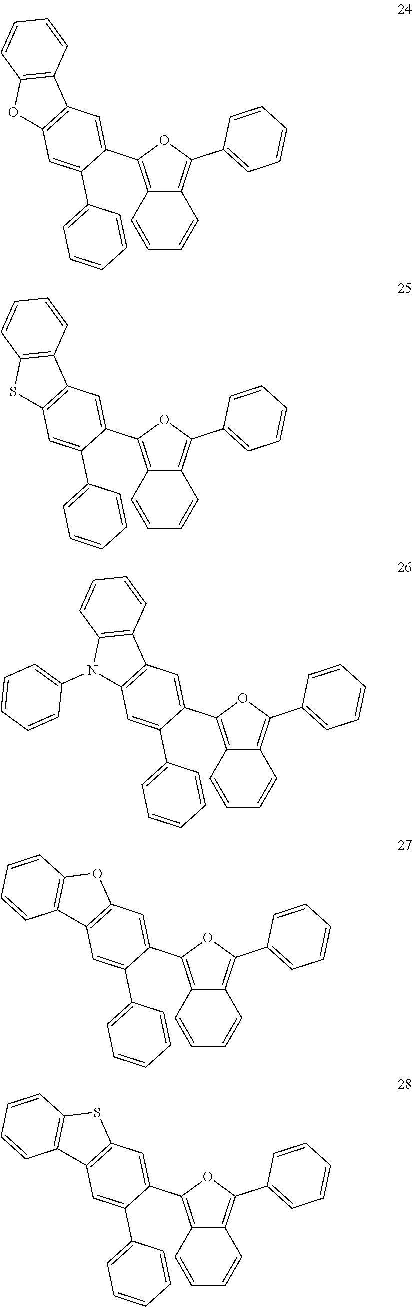

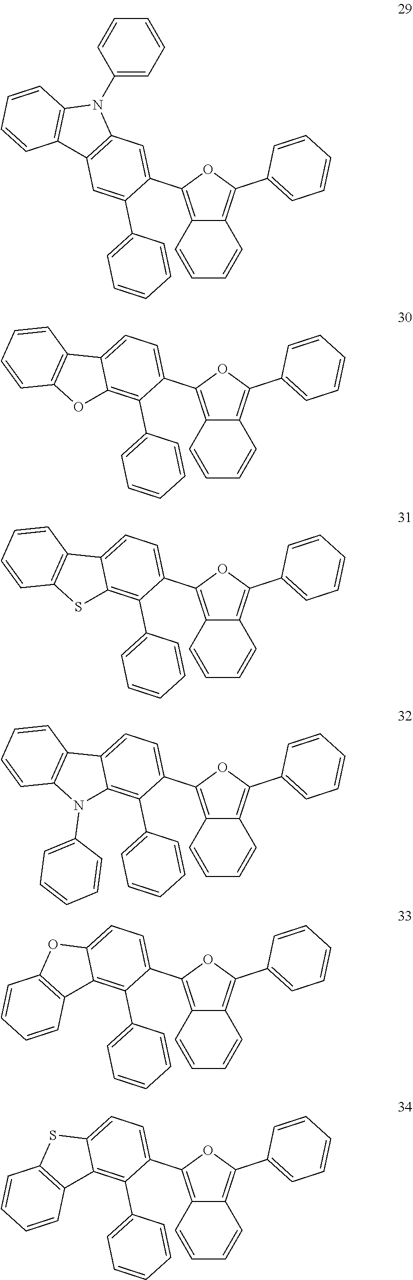

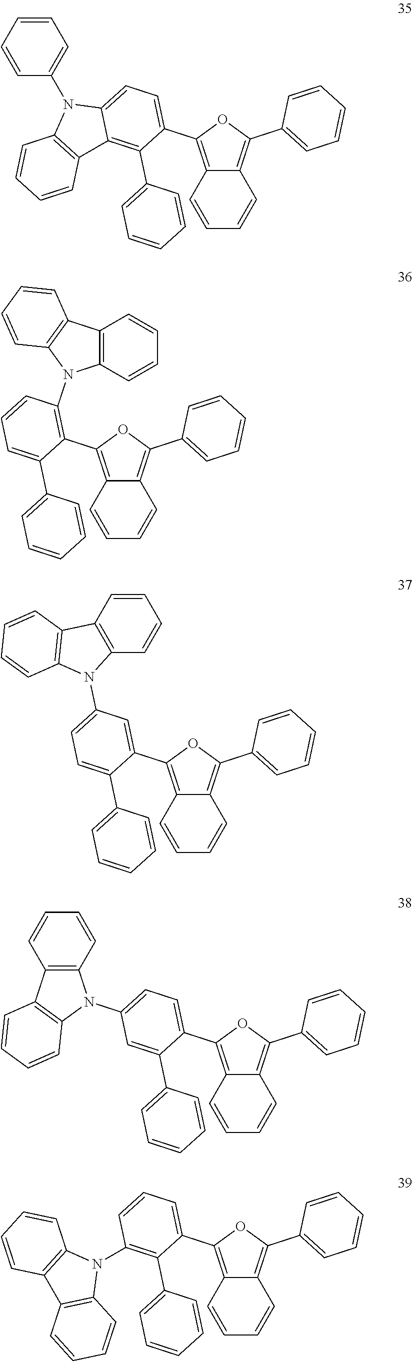 [SMILES: c1ccc(-c2oc(-c3ccc4c(c3-c3ccccc3)c3ccccc3n4-c3ccccc3)c3ccccc23)cc1 ; c1ccc(-c2cccc(-n3c4ccccc4c4ccccc43)c2-c2oc(-c3ccccc3)c3ccccc23)cc1 ; c1ccc(-c2ccc(-n3c4ccccc4c4ccccc43)cc2-c2oc(-c3ccccc3)c3ccccc23)cc1 ; c1ccc(-c2cc(-n3c4ccccc4c4ccccc43)ccc2-c2oc(-c3ccccc3)c3ccccc23)cc1 ; c1ccc(-c2c(-c3oc(-c4ccccc4)c4ccccc34)cccc2-n2c3ccccc3c3ccccc32)cc1]

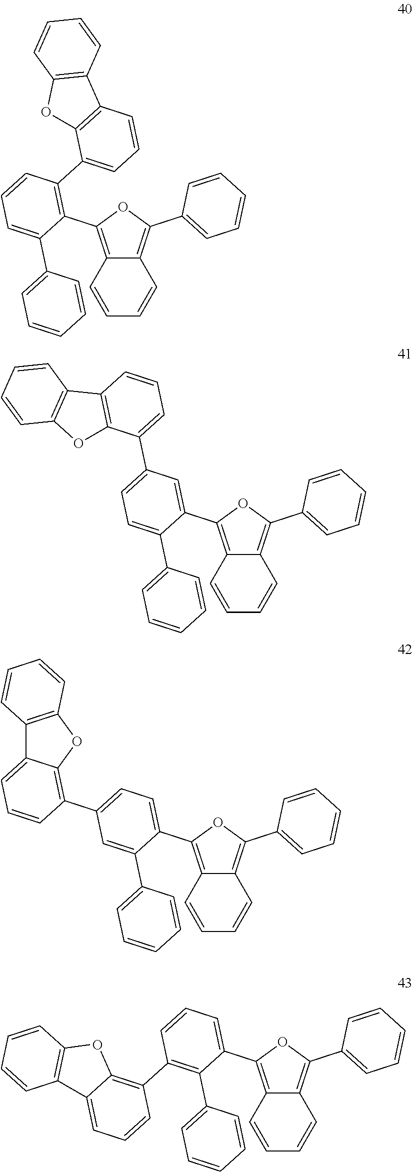

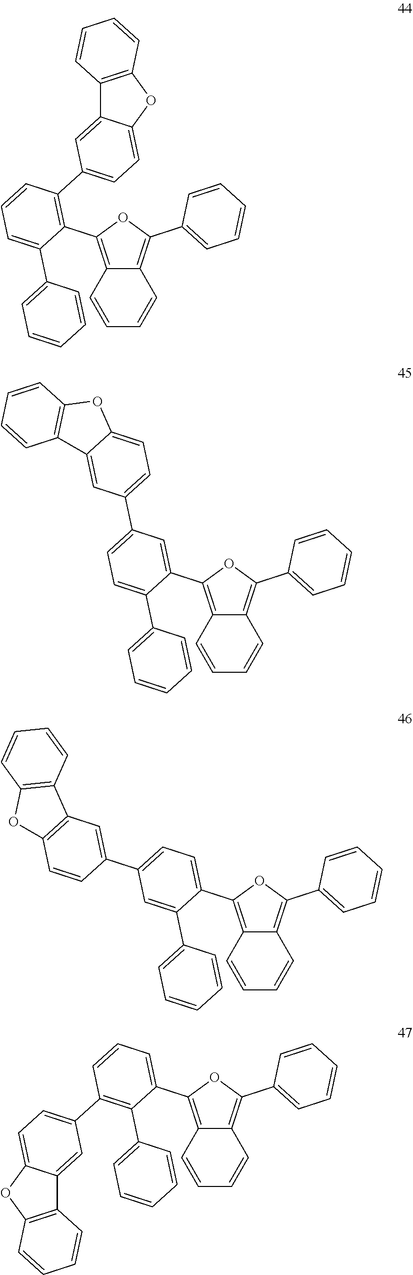

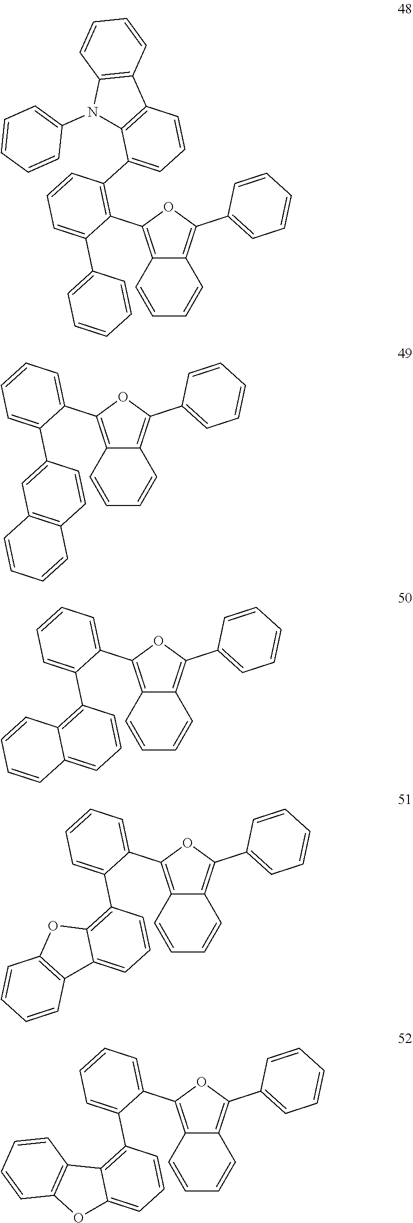

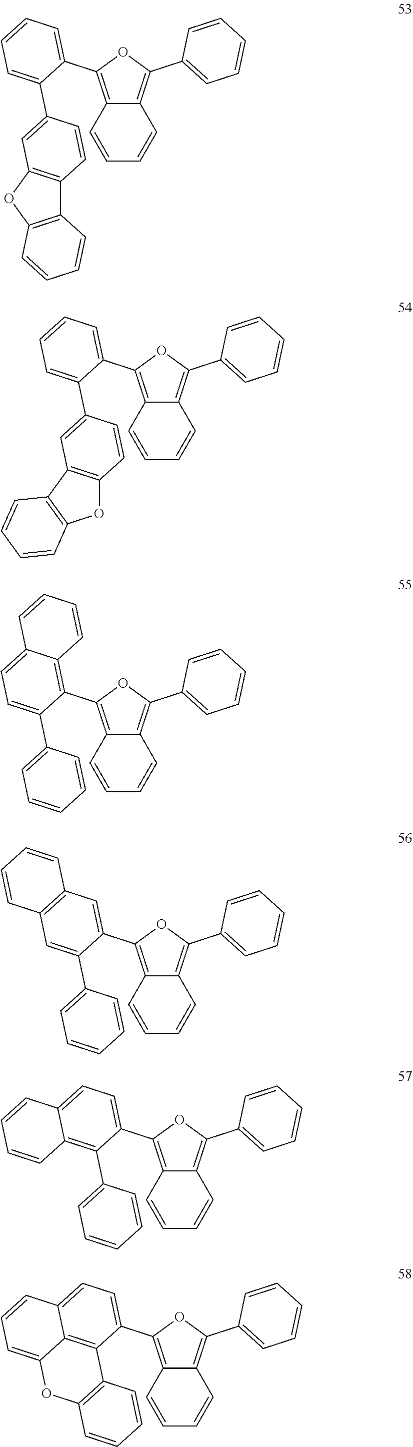

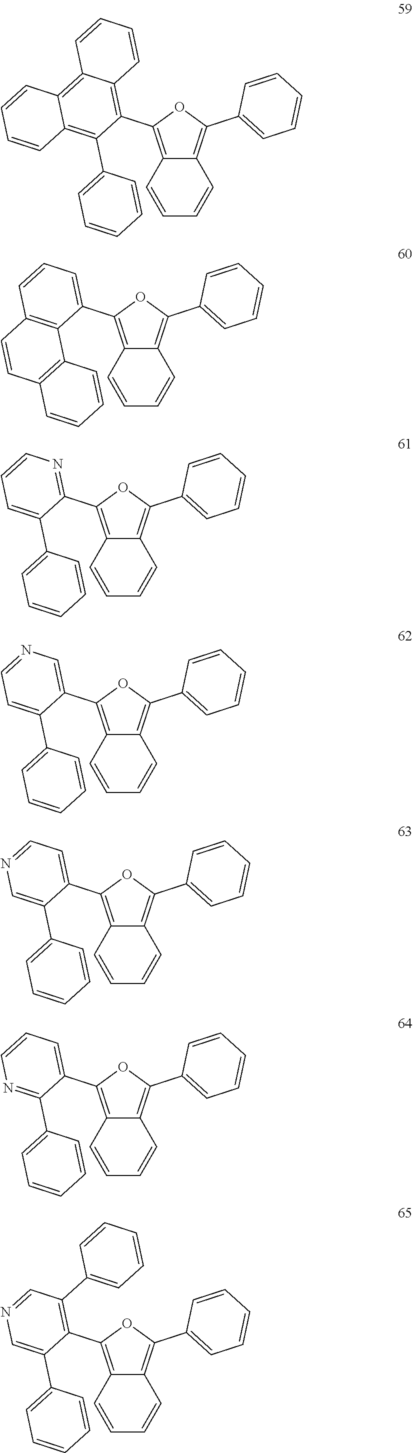

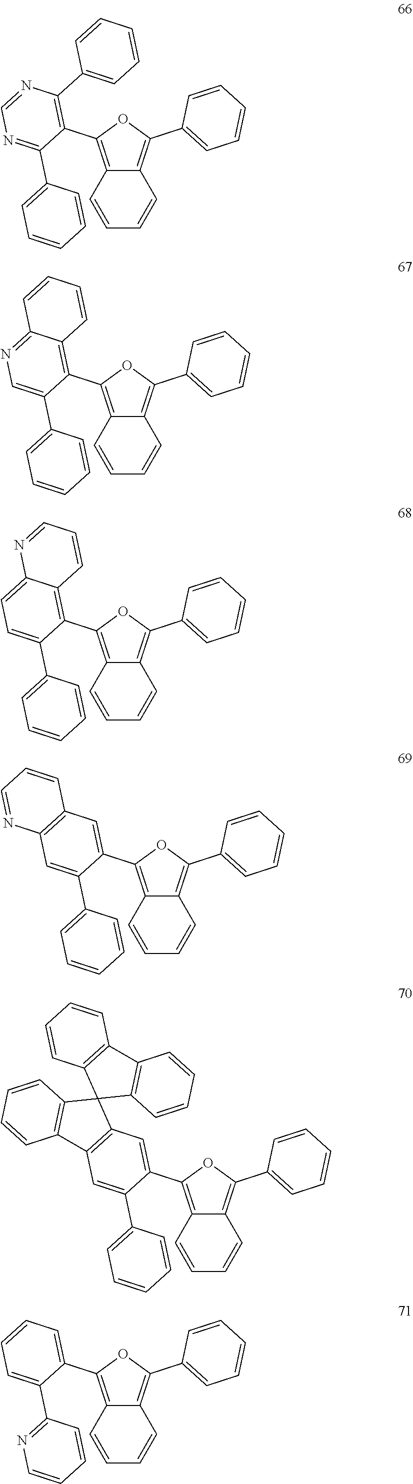

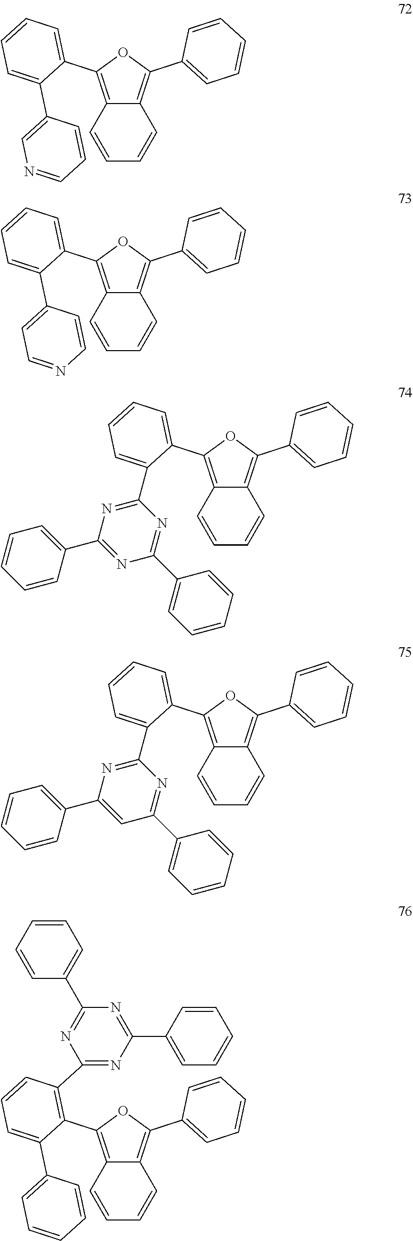

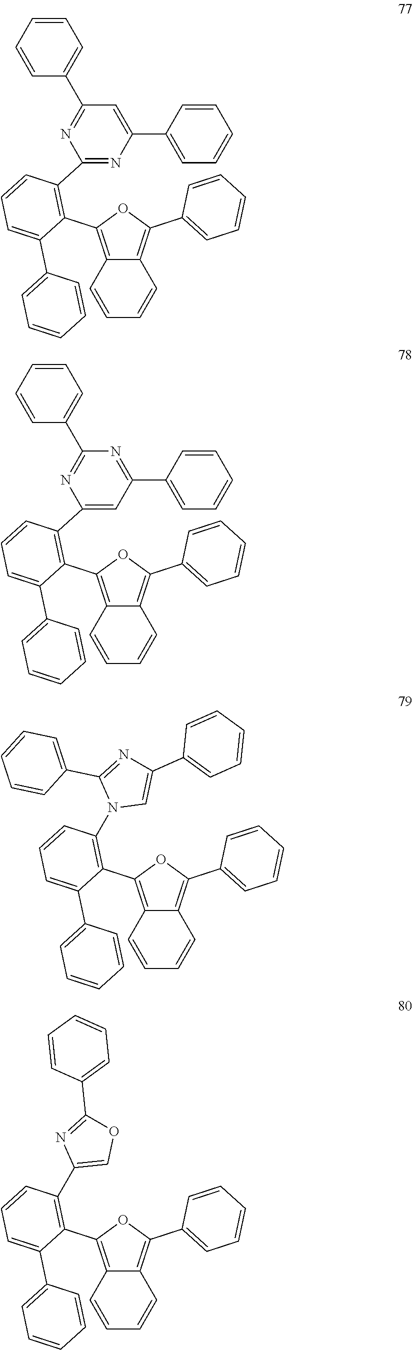

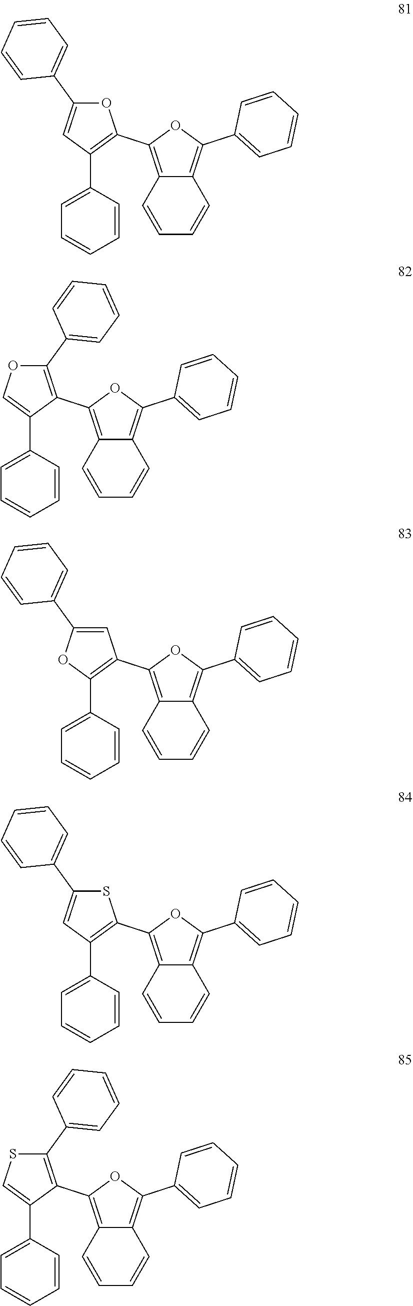

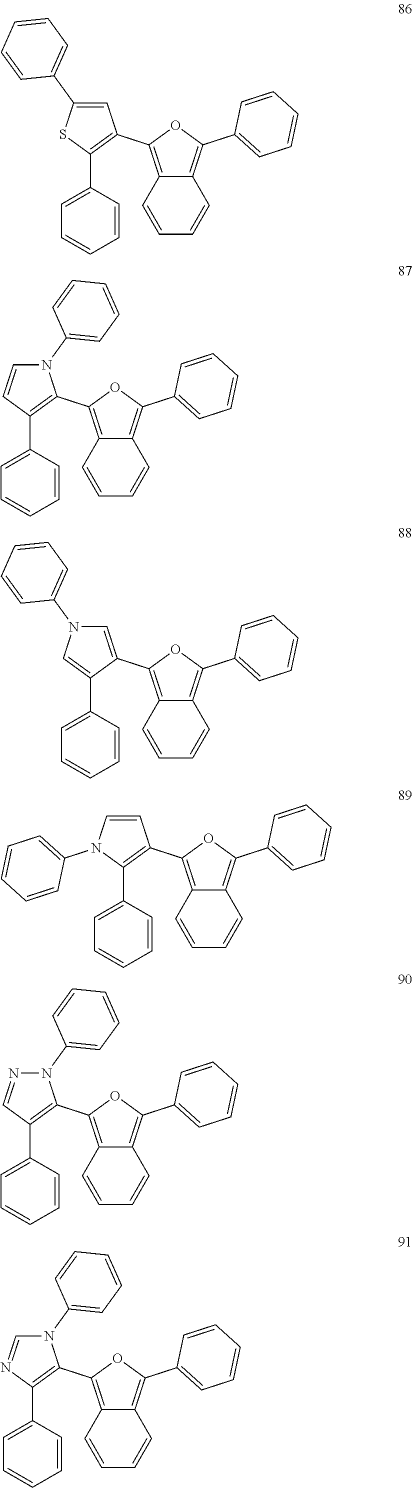

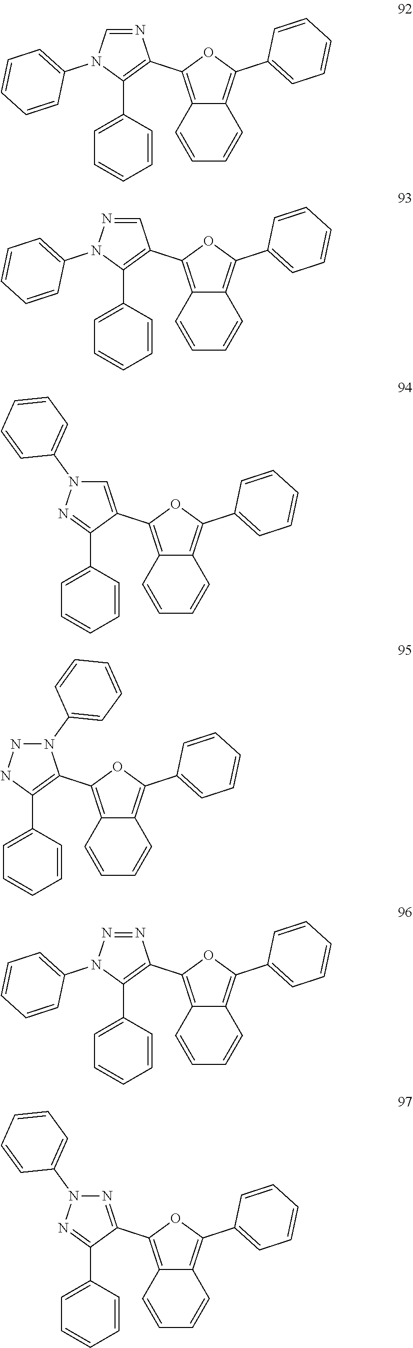

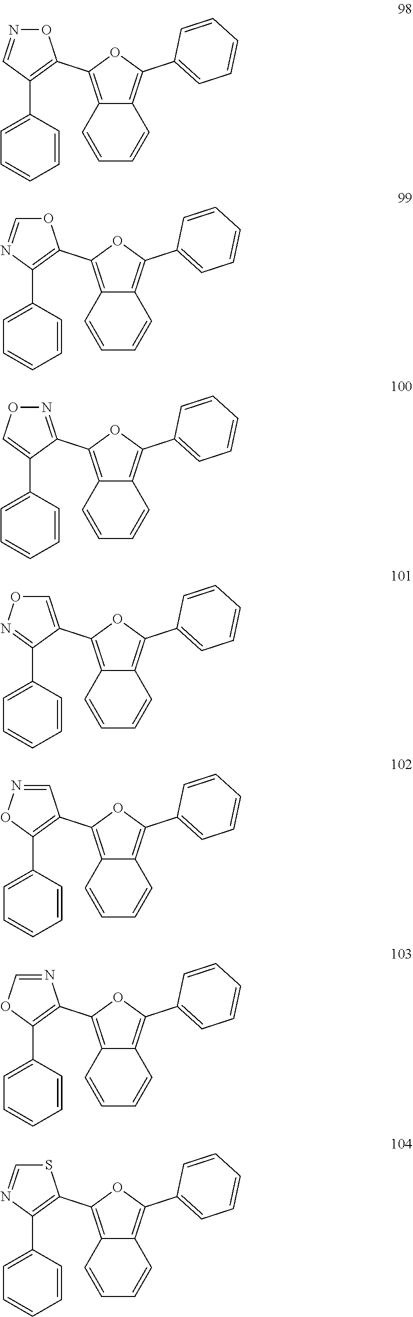

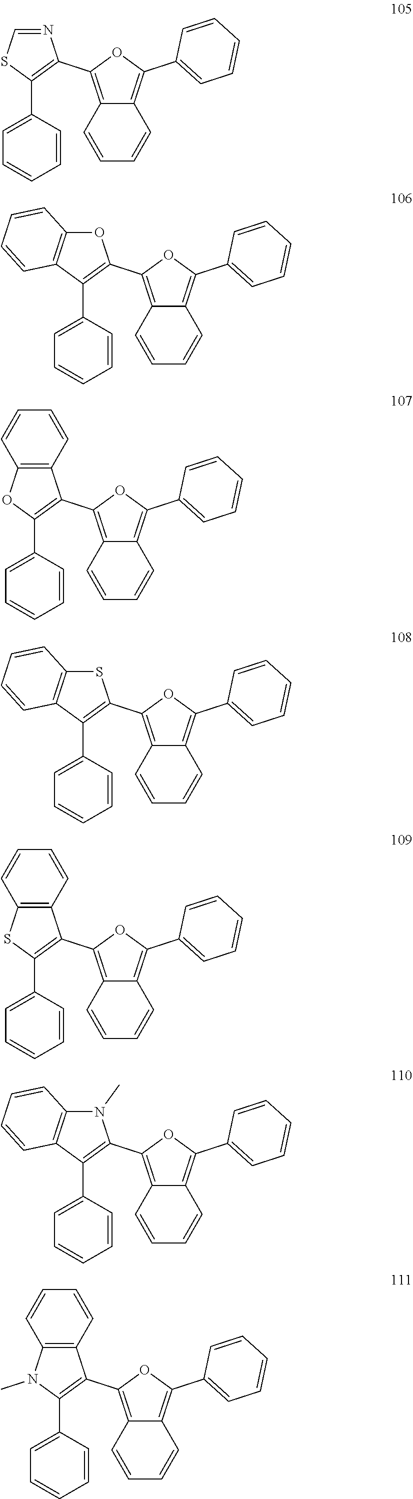

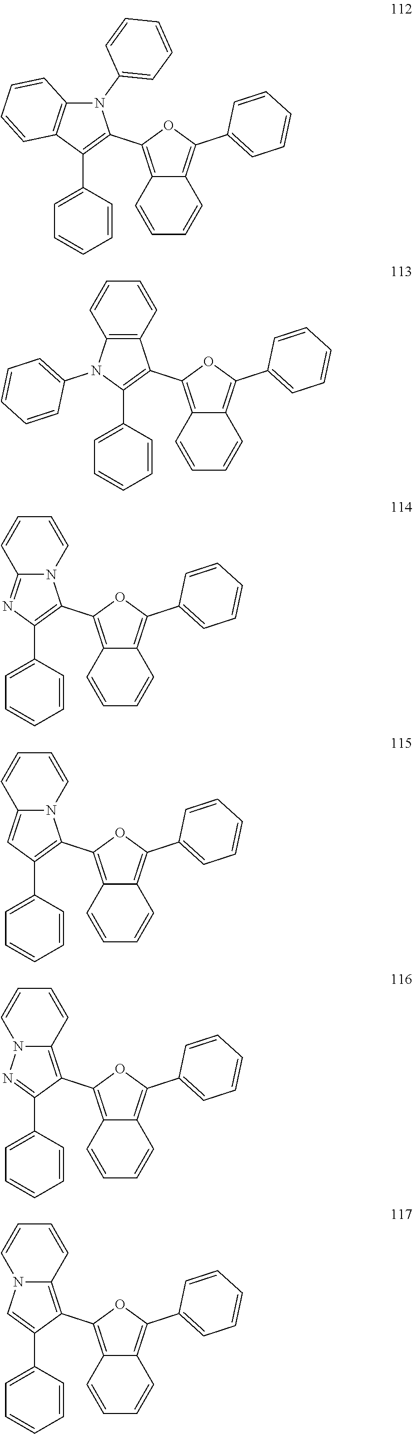

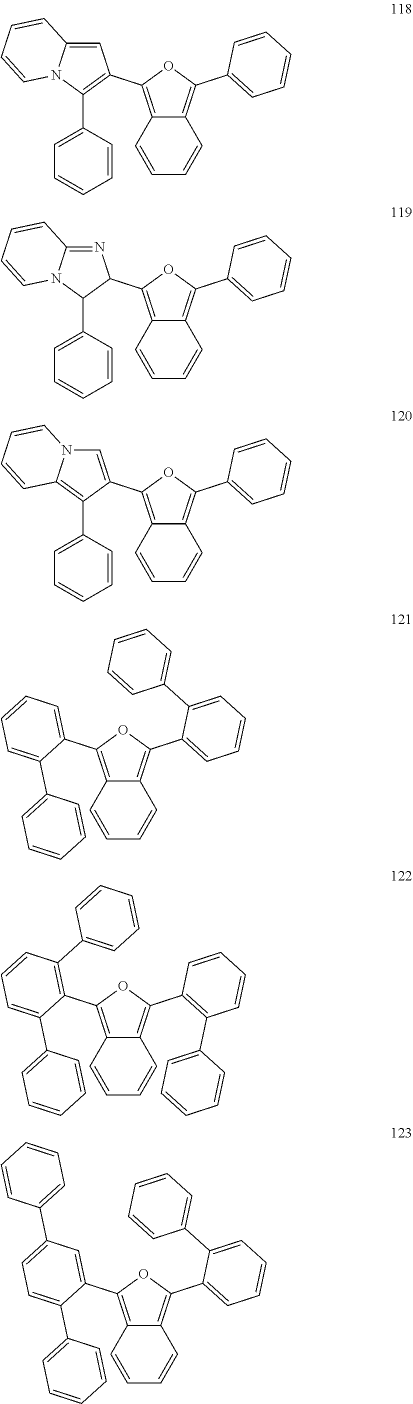

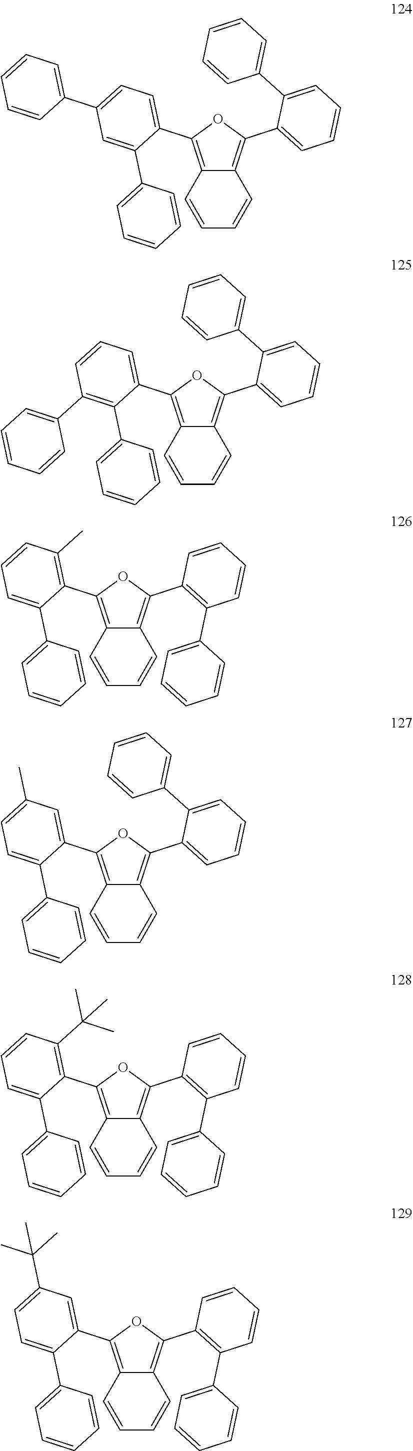

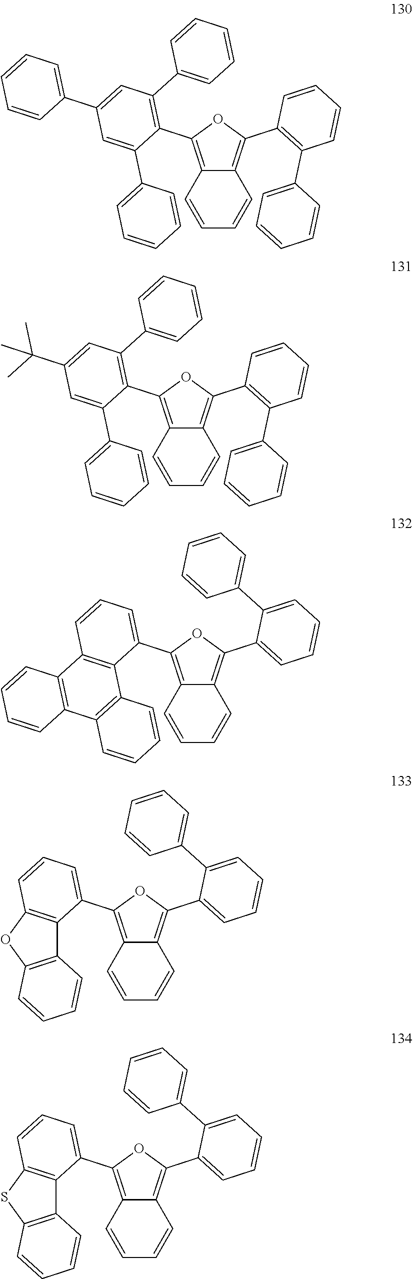

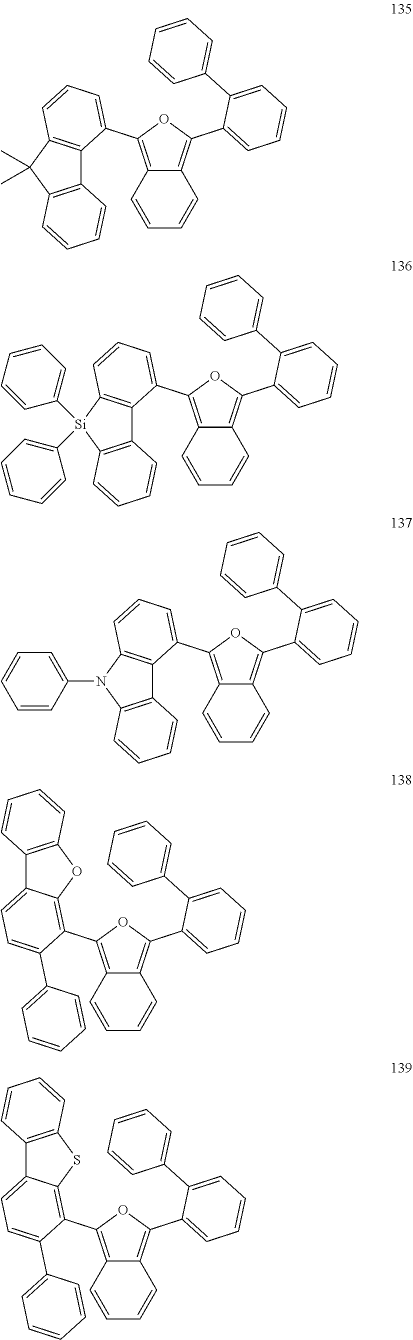

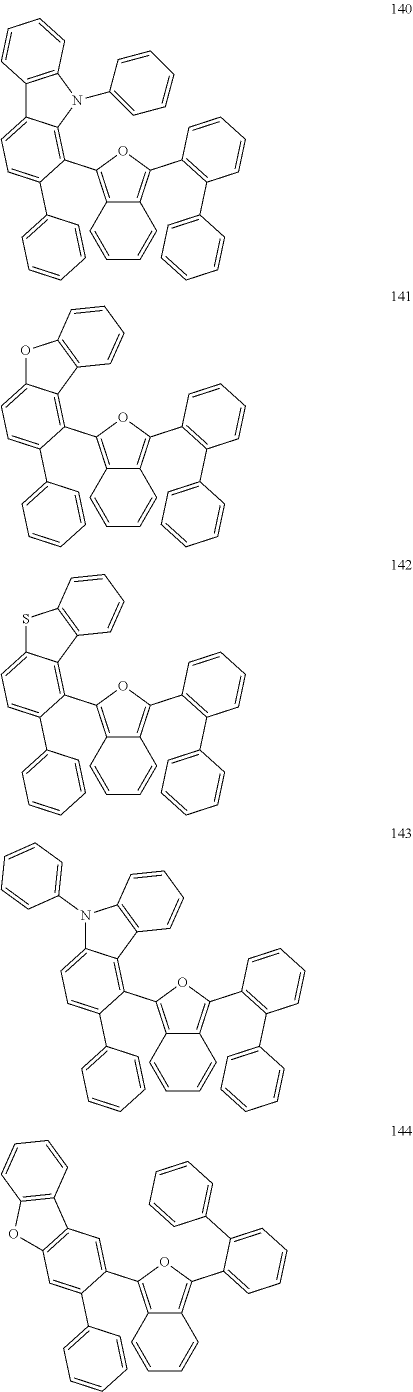

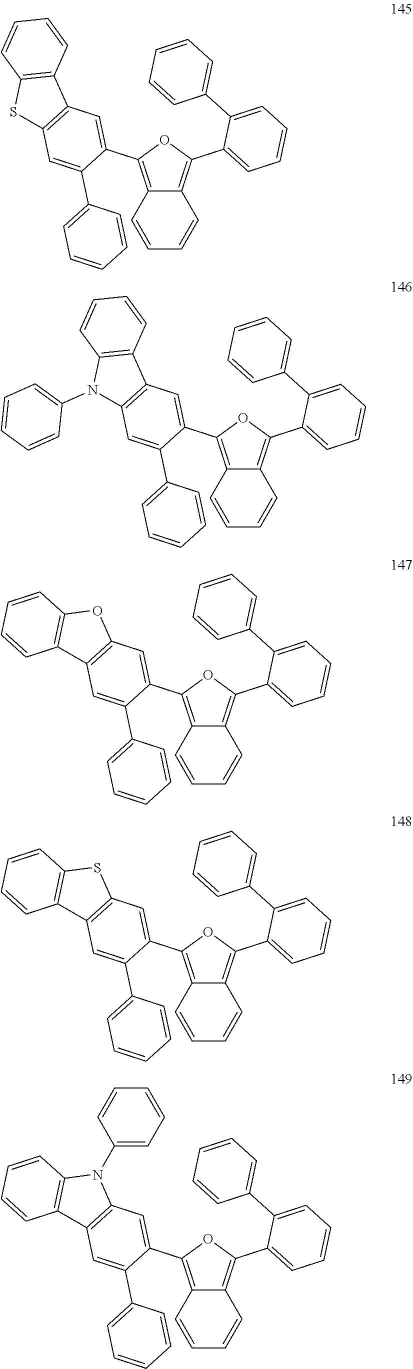

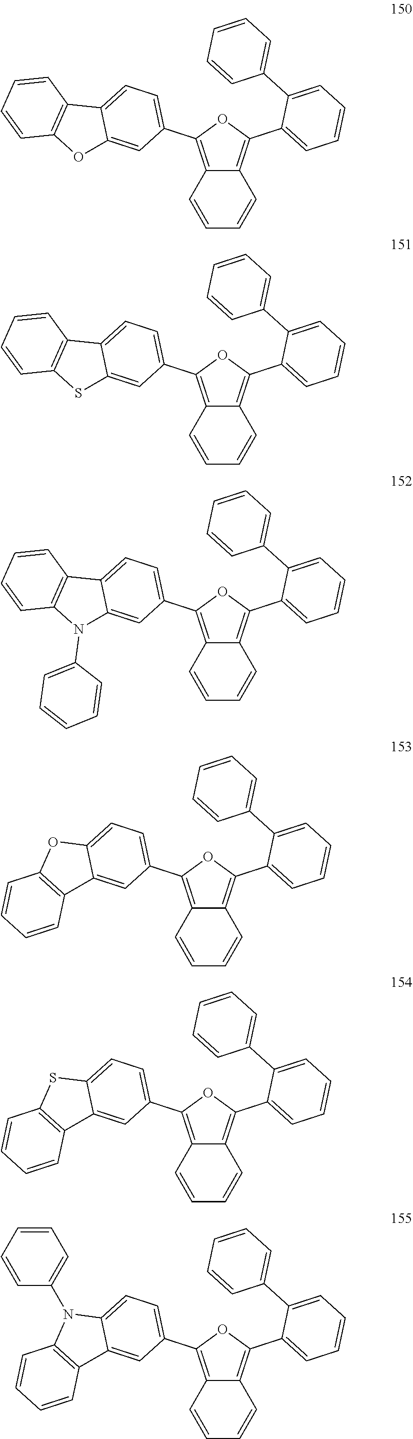

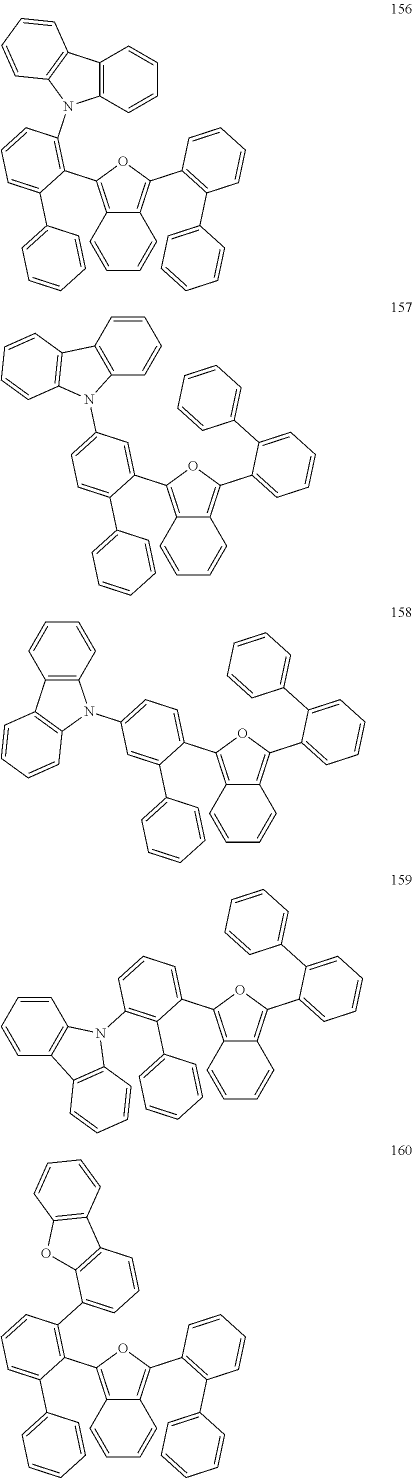

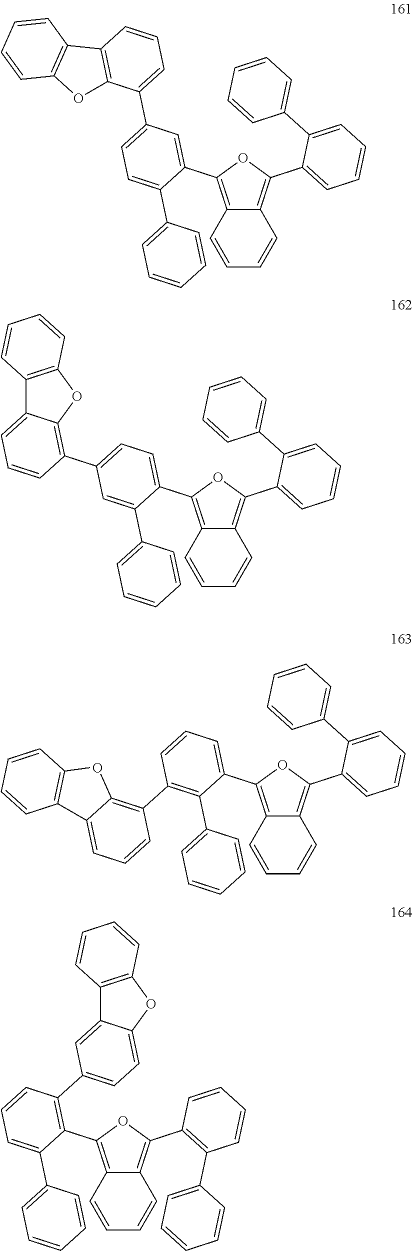 [SMILES: c1ccc(-c2ccccc2-c2oc(-c3cc(-c4cccc5c4oc4ccccc45)ccc3-c3ccccc3)c3ccccc23)cc1 ; c1ccc(-c2ccccc2-c2oc(-c3ccc(-c4cccc5c4oc4ccccc45)cc3-c3ccccc3)c3ccccc23)cc1 ; c1ccc(-c2ccccc2-c2oc(-c3cccc(-c4cccc5c4oc4ccccc45)c3-c3ccccc3)c3ccccc23)cc1 ; c1ccc(-c2ccccc2-c2oc(-c3c(-c4ccccc4)cccc3-c3ccc4oc5ccccc5c4c3)c3ccccc23)cc1]

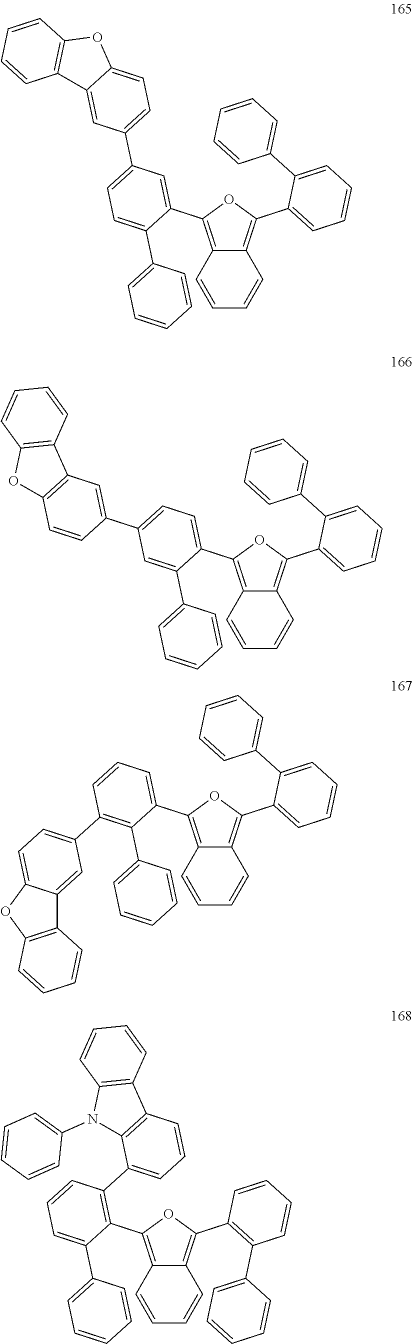

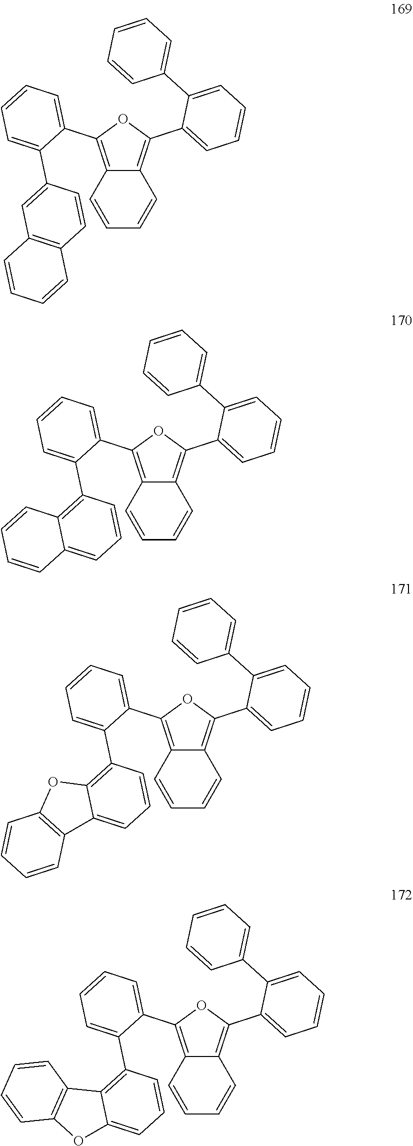 [SMILES: c1ccc(-c2ccccc2-c2oc(-c3ccccc3-c3ccc4ccccc4c3)c3ccccc23)cc1 ; c1ccc(-c2ccccc2-c2oc(-c3ccccc3-c3cccc4ccccc34)c3ccccc23)cc1 ; c1ccc(-c2ccccc2-c2oc(-c3ccccc3-c3cccc4c3oc3ccccc34)c3ccccc23)cc1 ; c1ccc(-c2ccccc2-c2oc(-c3ccccc3-c3cccc4oc5ccccc5c34)c3ccccc23)cc1]

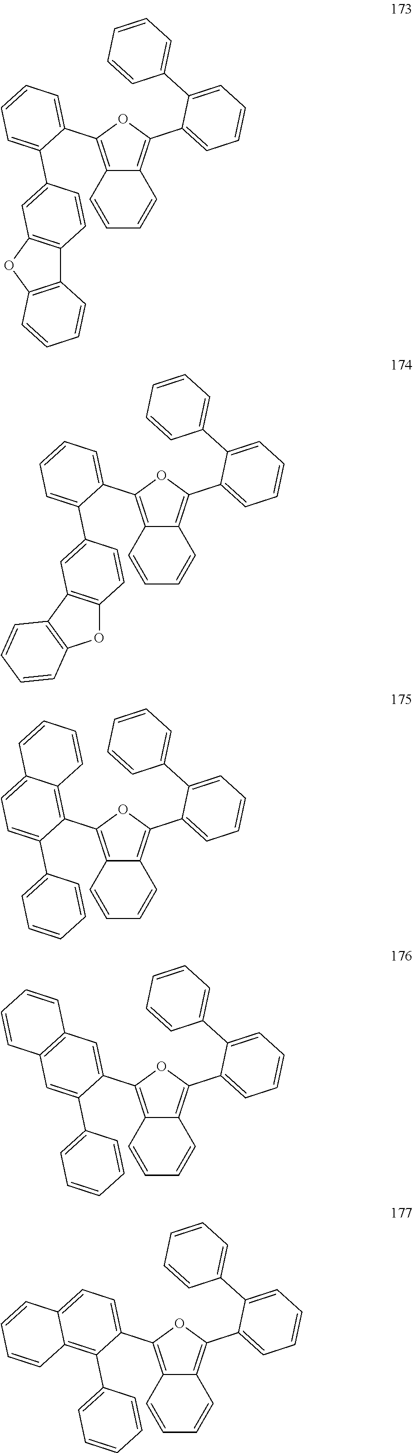

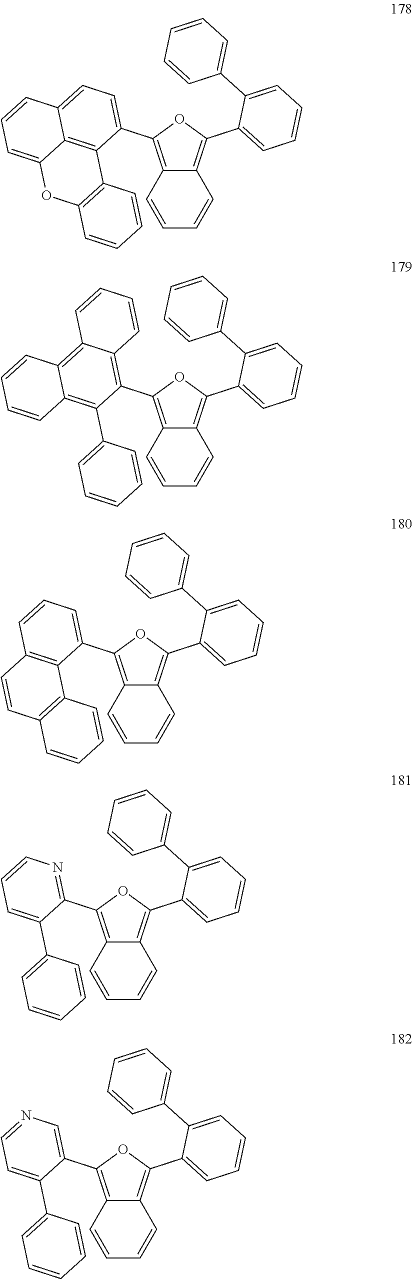

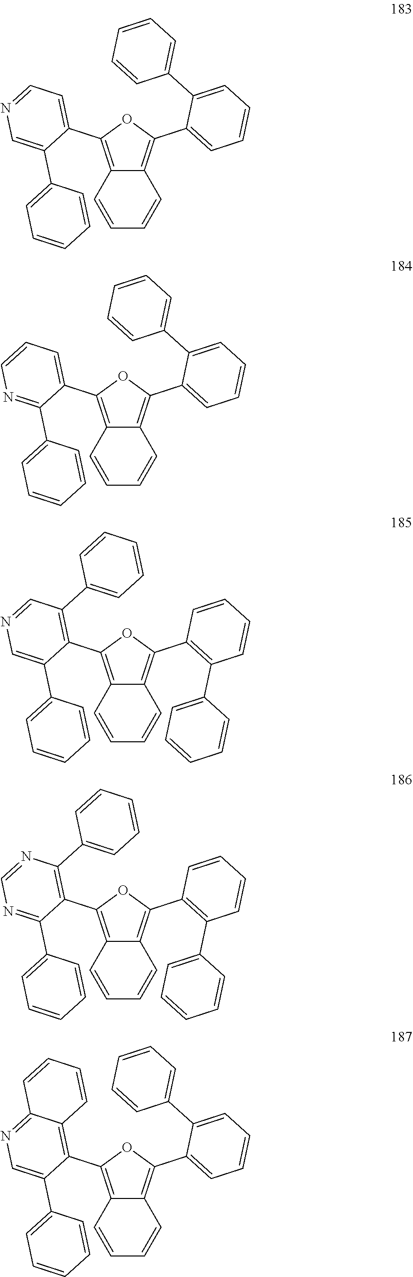

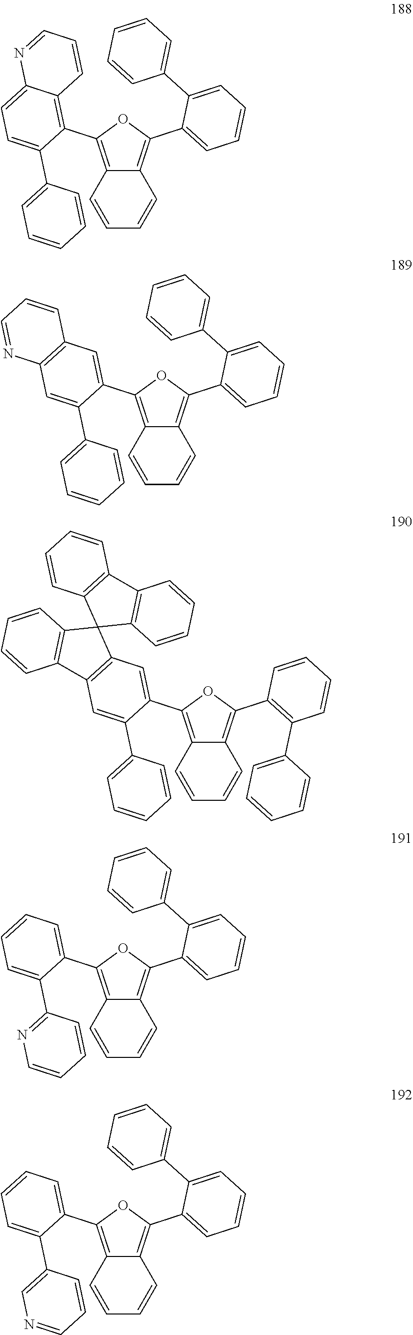

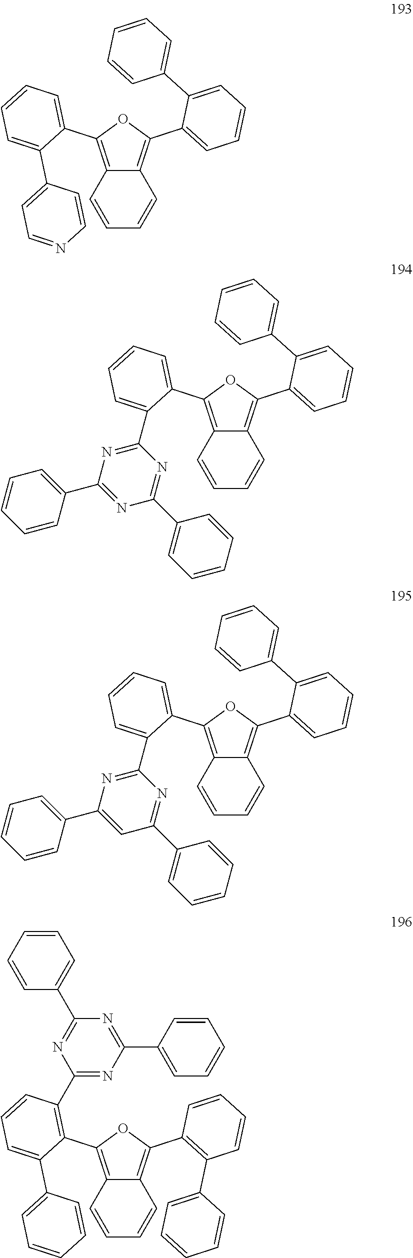

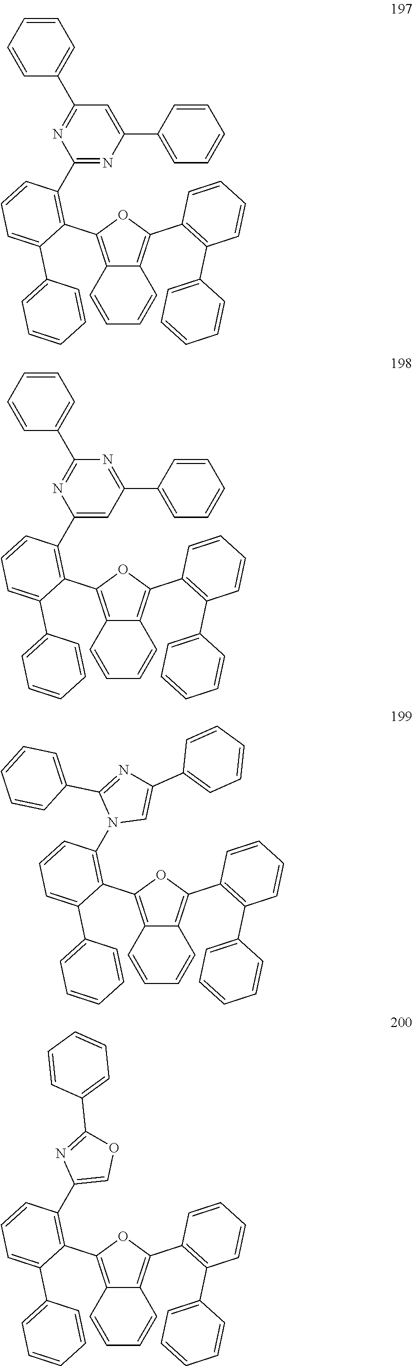

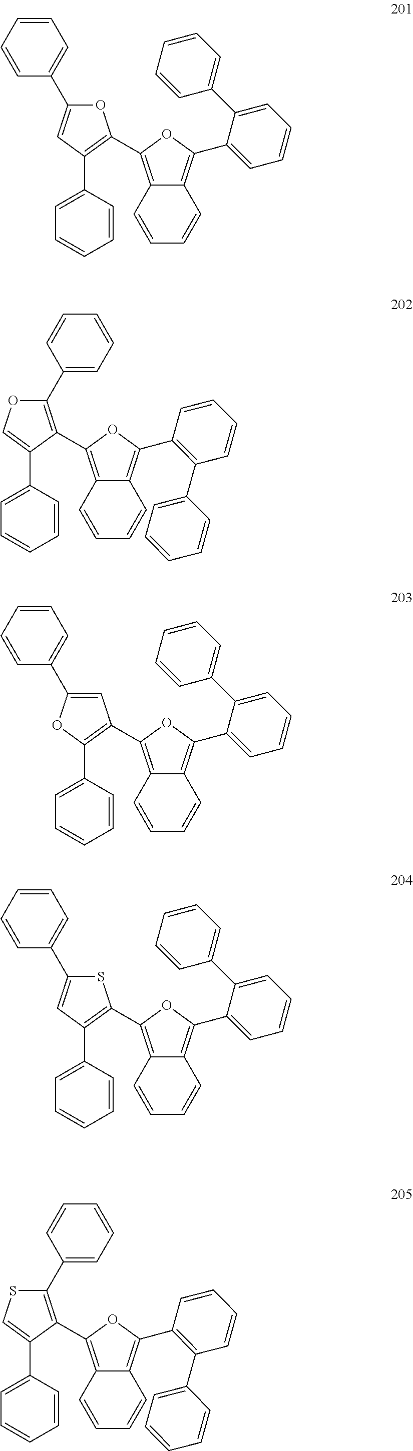 [SMILES: c1ccc(-c2cc(-c3ccccc3)c(-c3oc(-c4ccccc4-c4ccccc4)c4ccccc34)o2)cc1 ; c1ccc(-c2ccccc2-c2oc(-c3c(-c4ccccc4)coc3-c3ccccc3)c3ccccc23)cc1 ; c1ccc(-c2cc(-c3oc(-c4ccccc4-c4ccccc4)c4ccccc34)c(-c3ccccc3)o2)cc1 ; c1ccc(-c2cc(-c3ccccc3)c(-c3oc(-c4ccccc4-c4ccccc4)c4ccccc34)s2)cc1 ; c1ccc(-c2ccccc2-c2oc(-c3c(-c4ccccc4)csc3-c3ccccc3)c3ccccc23)cc1]

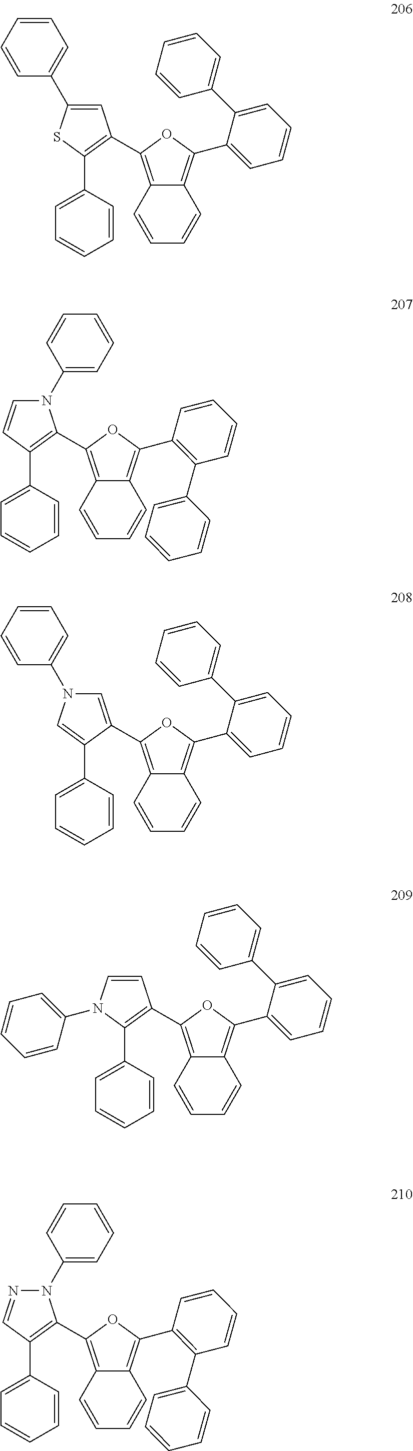 [SMILES: c1ccc(-c2cc(-c3oc(-c4ccccc4-c4ccccc4)c4ccccc34)c(-c3ccccc3)s2)cc1 ; c1ccc(-c2ccccc2-c2oc(-c3c(-c4ccccc4)ccn3-c3ccccc3)c3ccccc23)cc1 ; c1ccc(-c2ccccc2-c2oc(-c3cn(-c4ccccc4)cc3-c3ccccc3)c3ccccc23)cc1 ; c1ccc(-c2ccccc2-c2oc(-c3ccn(-c4ccccc4)c3-c3ccccc3)c3ccccc23)cc1 ; c1ccc(-c2ccccc2-c2oc(-c3c(-c4ccccc4)cnn3-c3ccccc3)c3ccccc23)cc1]

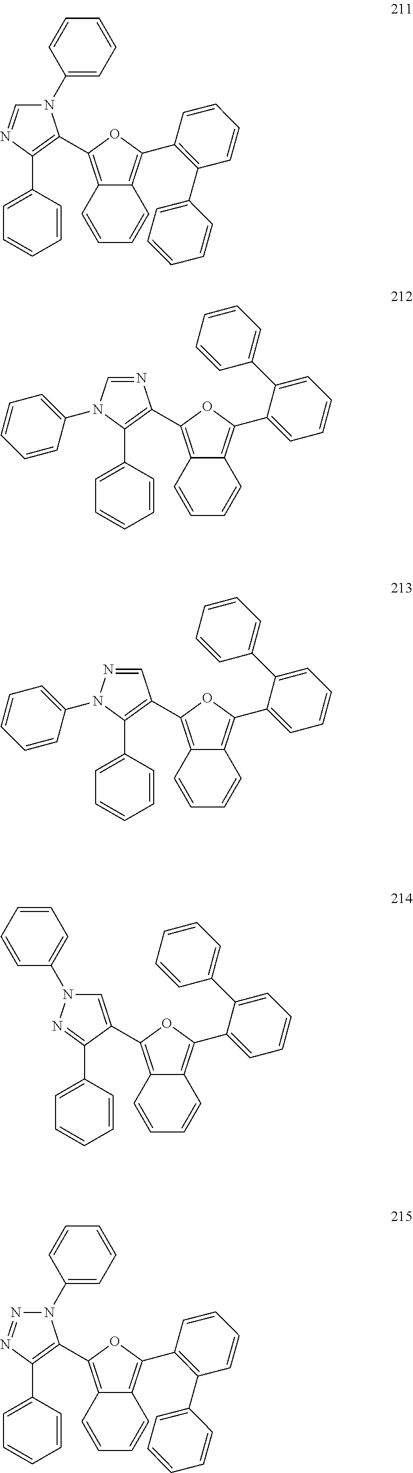 [SMILES: c1ccc(-c2ccccc2-c2oc(-c3c(-c4ccccc4)ncn3-c3ccccc3)c3ccccc23)cc1 ; c1ccc(-c2ccccc2-c2oc(-c3ncn(-c4ccccc4)c3-c3ccccc3)c3ccccc23)cc1 ; c1ccc(-c2ccccc2-c2oc(-c3cnn(-c4ccccc4)c3-c3ccccc3)c3ccccc23)cc1 ; c1ccc(-c2ccccc2-c2oc(-c3cn(-c4ccccc4)nc3-c3ccccc3)c3ccccc23)cc1 ; c1ccc(-c2ccccc2-c2oc(-c3c(-c4ccccc4)nnn3-c3ccccc3)c3ccccc23)cc1]

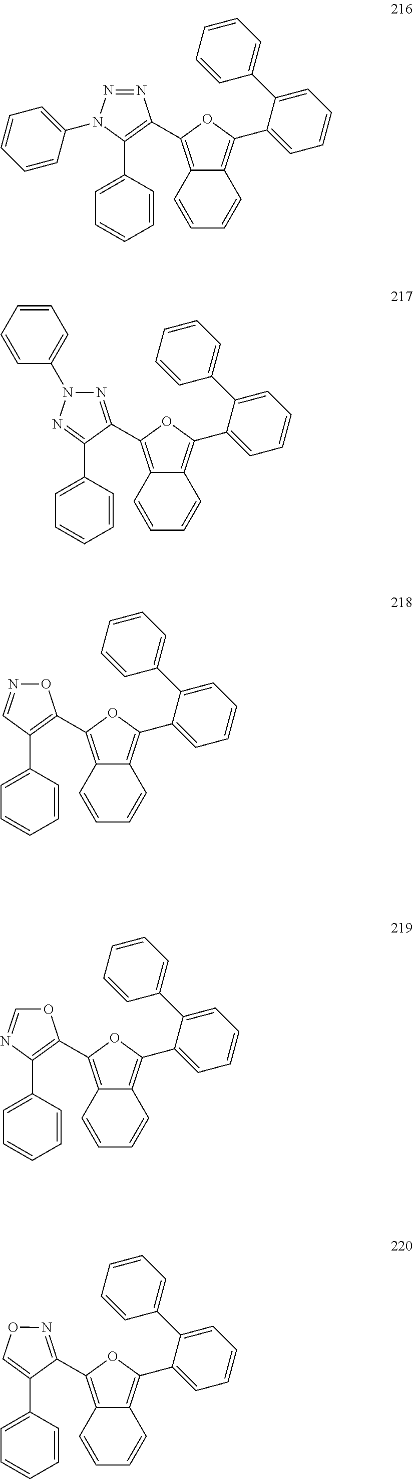 [SMILES: c1ccc(-c2ccccc2-c2oc(-c3nnn(-c4ccccc4)c3-c3ccccc3)c3ccccc23)cc1 ; c1ccc(-c2ccccc2-c2oc(-c3nn(-c4ccccc4)nc3-c3ccccc3)c3ccccc23)cc1 ; c1ccc(-c2ccccc2-c2oc(-c3oncc3-c3ccccc3)c3ccccc23)cc1 ; c1ccc(-c2ccccc2-c2oc(-c3ocnc3-c3ccccc3)c3ccccc23)cc1 ; c1ccc(-c2ccccc2-c2oc(-c3nocc3-c3ccccc3)c3ccccc23)cc1]

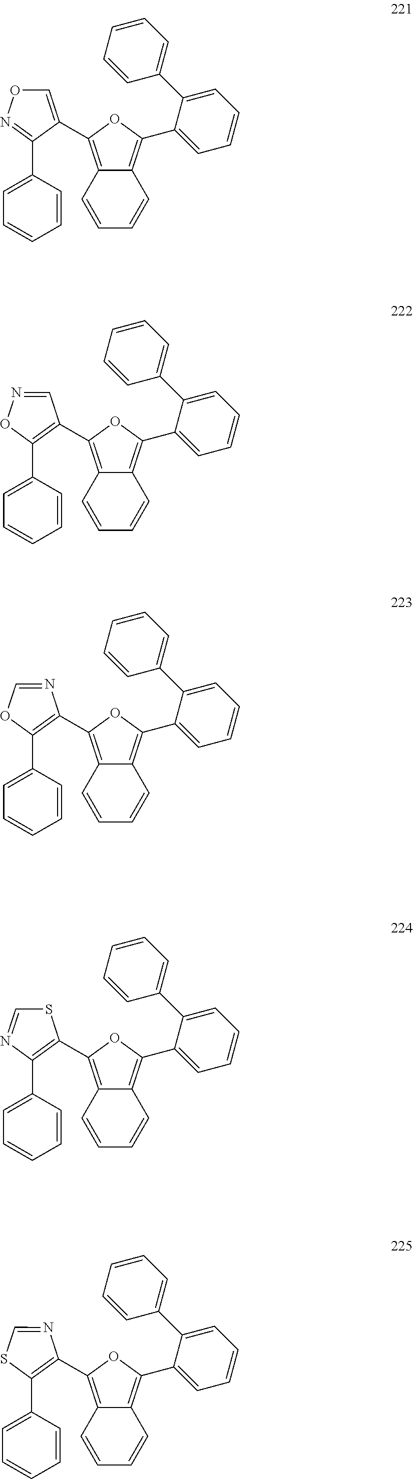 [SMILES: c1ccc(-c2ccccc2-c2oc(-c3conc3-c3ccccc3)c3ccccc23)cc1 ; c1ccc(-c2ccccc2-c2oc(-c3cnoc3-c3ccccc3)c3ccccc23)cc1 ; c1ccc(-c2ccccc2-c2oc(-c3ncoc3-c3ccccc3)c3ccccc23)cc1 ; c1ccc(-c2ccccc2-c2oc(-c3scnc3-c3ccccc3)c3ccccc23)cc1 ; c1ccc(-c2ccccc2-c2oc(-c3ncsc3-c3ccccc3)c3ccccc23)cc1]

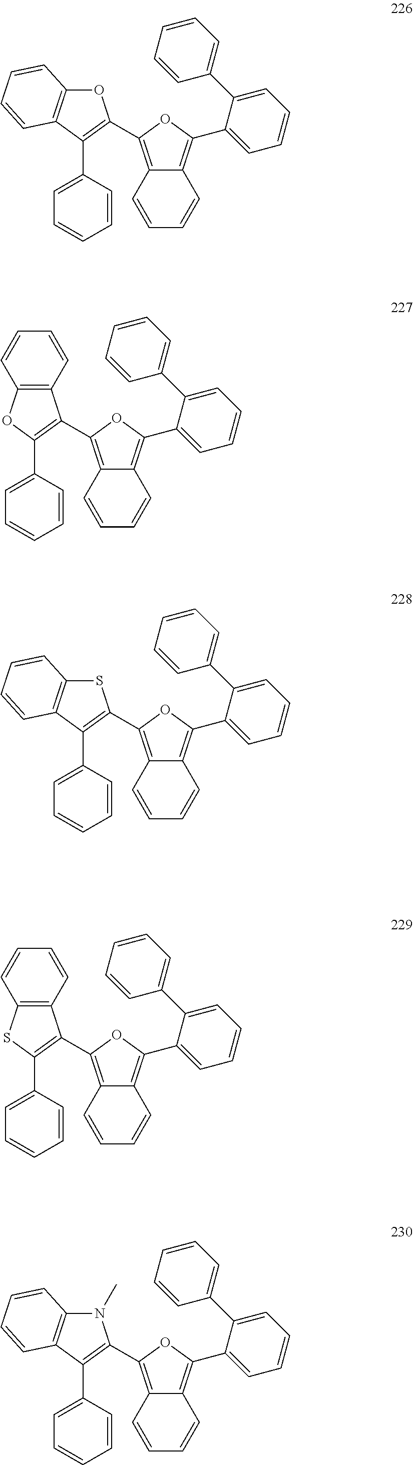 [SMILES: c1ccc(-c2ccccc2-c2oc(-c3oc4ccccc4c3-c3ccccc3)c3ccccc23)cc1 ; c1ccc(-c2ccccc2-c2oc(-c3c(-c4ccccc4)oc4ccccc34)c3ccccc23)cc1 ; c1ccc(-c2ccccc2-c2oc(-c3sc4ccccc4c3-c3ccccc3)c3ccccc23)cc1 ; c1ccc(-c2ccccc2-c2oc(-c3c(-c4ccccc4)sc4ccccc34)c3ccccc23)cc1 ; Cn1c(-c2oc(-c3ccccc3-c3ccccc3)c3ccccc23)c(-c2ccccc2)c2ccccc21]

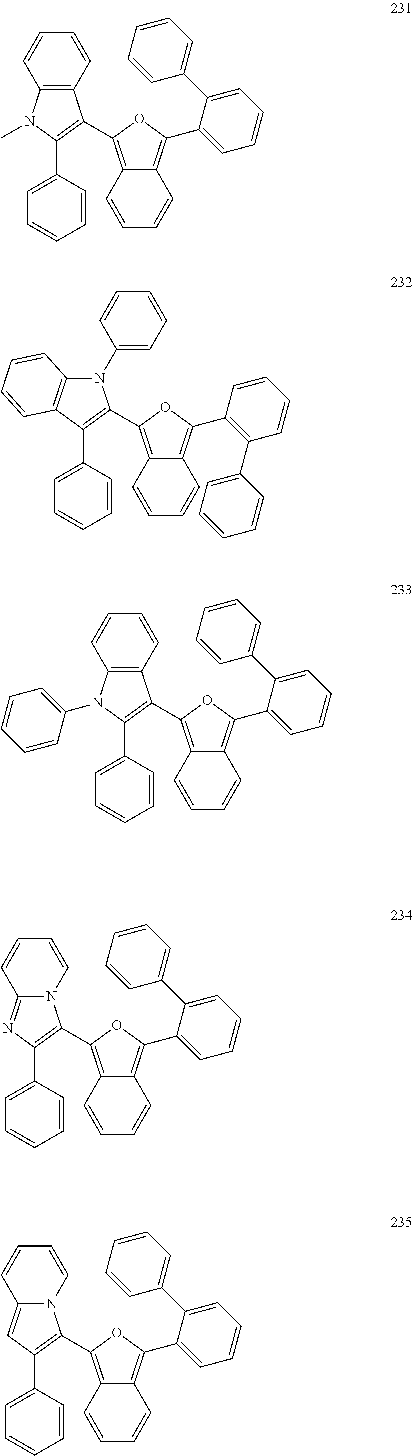 [SMILES: Cn1c(-c2ccccc2)c(-c2oc(-c3ccccc3-c3ccccc3)c3ccccc23)c2ccccc21 ; c1ccc(-c2ccccc2-c2oc(-c3c(-c4ccccc4)c4ccccc4n3-c3ccccc3)c3ccccc23)cc1 ; c1ccc(-c2ccccc2-c2oc(-c3c(-c4ccccc4)n(-c4ccccc4)c4ccccc34)c3ccccc23)cc1 ; c1ccc(-c2ccccc2-c2oc(-c3c(-c4ccccc4)nc4ccccn34)c3ccccc23)cc1 ; c1ccc(-c2ccccc2-c2oc(-c3c(-c4ccccc4)cc4ccccn34)c3ccccc23)cc1]

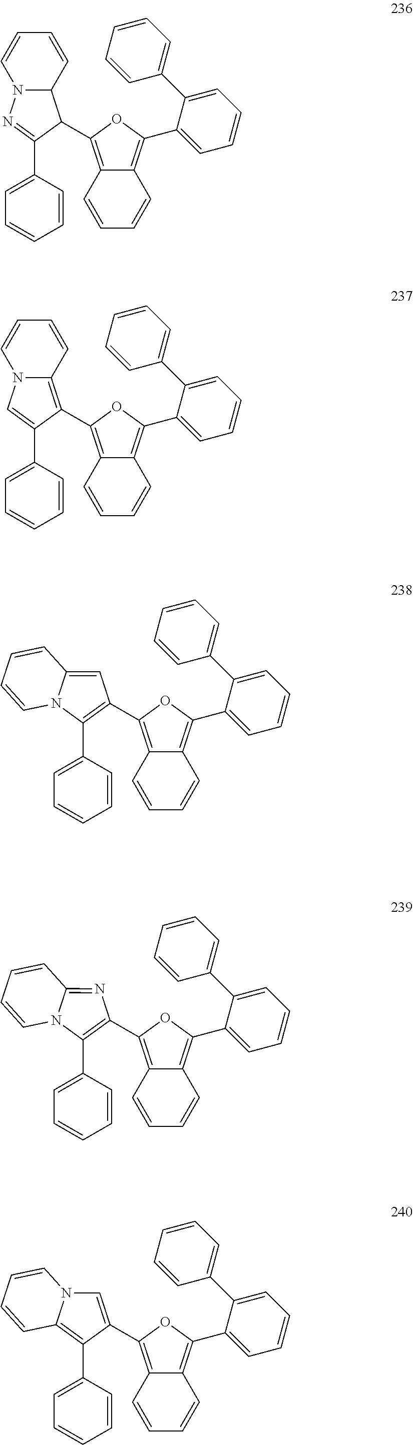 [SMILES: C1=CC2C(c3oc(-c4ccccc4-c4ccccc4)c4ccccc34)C(c3ccccc3)=NN2C=C1 ; c1ccc(-c2ccccc2-c2oc(-c3c(-c4ccccc4)cn4ccccc34)c3ccccc23)cc1 ; c1ccc(-c2ccccc2-c2oc(-c3cc4ccccn4c3-c3ccccc3)c3ccccc23)cc1 ; c1ccc(-c2ccccc2-c2oc(-c3nc4ccccn4c3-c3ccccc3)c3ccccc23)cc1 ; c1ccc(-c2ccccc2-c2oc(-c3cn4ccccc4c3-c3ccccc3)c3ccccc23)cc1]

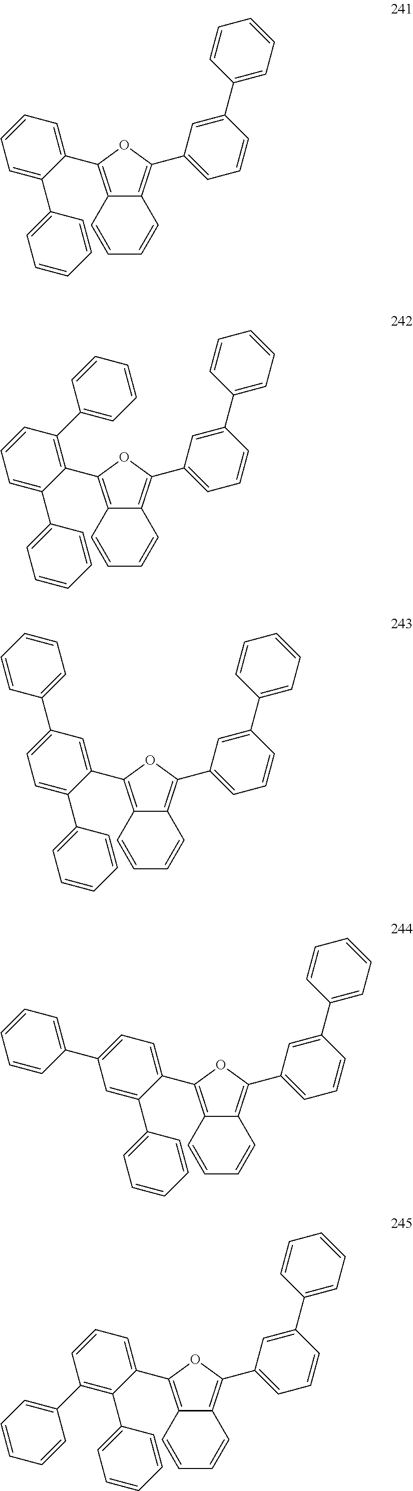 [SMILES: c1ccc(-c2cccc(-c3oc(-c4ccccc4-c4ccccc4)c4ccccc34)c2)cc1 ; c1ccc(-c2cccc(-c3oc(-c4c(-c5ccccc5)cccc4-c4ccccc4)c4ccccc34)c2)cc1 ; c1ccc(-c2cccc(-c3oc(-c4cc(-c5ccccc5)ccc4-c4ccccc4)c4ccccc34)c2)cc1 ; c1ccc(-c2cccc(-c3oc(-c4ccc(-c5ccccc5)cc4-c4ccccc4)c4ccccc34)c2)cc1 ; c1ccc(-c2cccc(-c3oc(-c4cccc(-c5ccccc5)c4-c4ccccc4)c4ccccc34)c2)cc1]

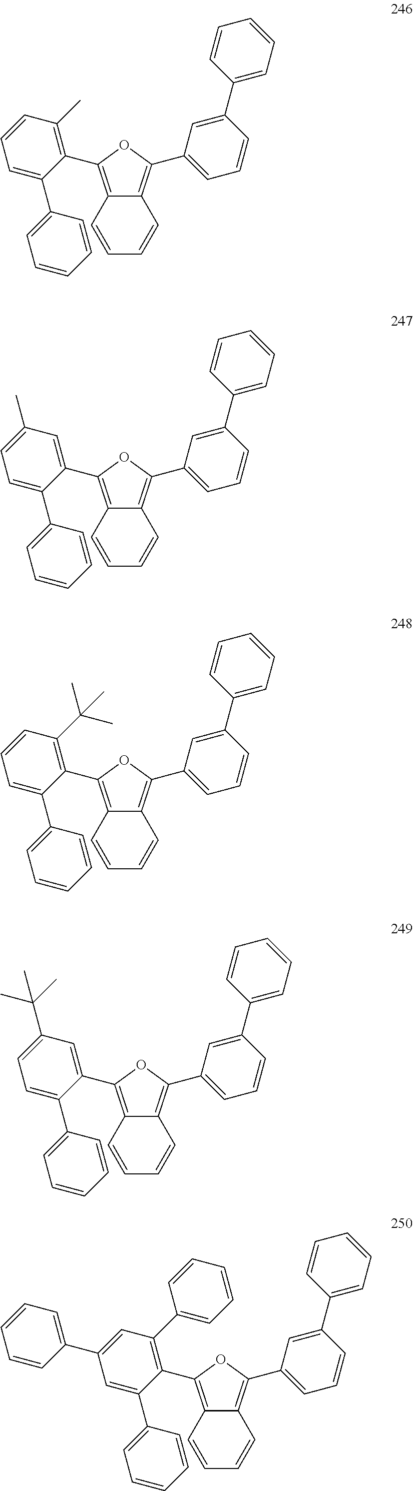 [SMILES: Cc1cccc(-c2ccccc2)c1-c1oc(-c2cccc(-c3ccccc3)c2)c2ccccc12 ; Cc1ccc(-c2ccccc2)c(-c2oc(-c3cccc(-c4ccccc4)c3)c3ccccc23)c1 ; CC(C)(C)c1cccc(-c2ccccc2)c1-c1oc(-c2cccc(-c3ccccc3)c2)c2ccccc12 ; CC(C)(C)c1ccc(-c2ccccc2)c(-c2oc(-c3cccc(-c4ccccc4)c3)c3ccccc23)c1 ; c1ccc(-c2cccc(-c3oc(-c4c(-c5ccccc5)cc(-c5ccccc5)cc4-c4ccccc4)c4ccccc34)c2)cc1]

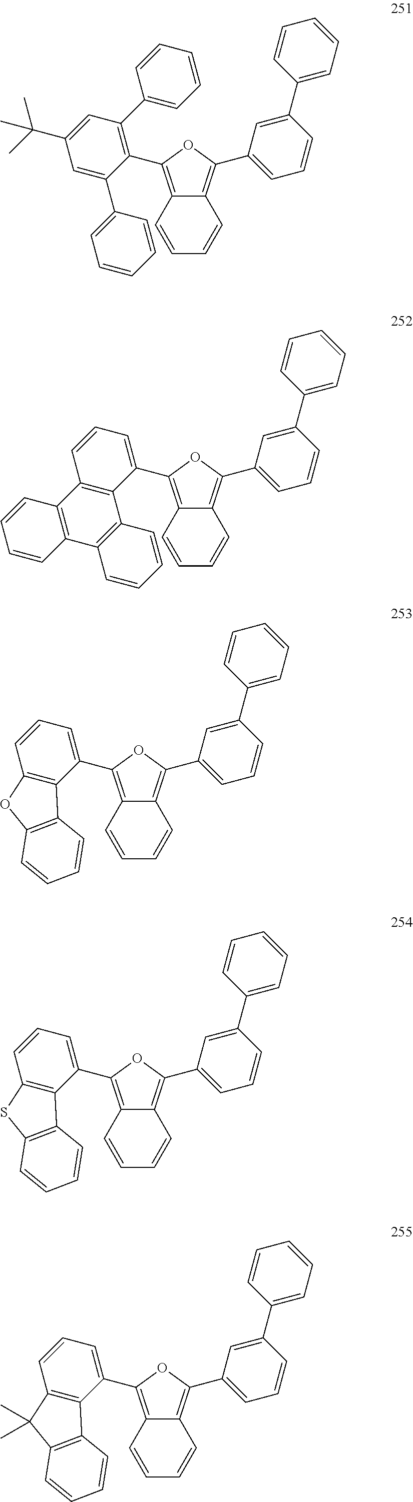 [SMILES: CC(C)(C)c1cc(-c2ccccc2)c(-c2oc(-c3cccc(-c4ccccc4)c3)c3ccccc23)c(-c2ccccc2)c1 ; c1ccc(-c2cccc(-c3oc(-c4cccc5c6ccccc6c6ccccc6c45)c4ccccc34)c2)cc1 ; c1ccc(-c2cccc(-c3oc(-c4cccc5oc6ccccc6c45)c4ccccc34)c2)cc1 ; c1ccc(-c2cccc(-c3oc(-c4cccc5sc6ccccc6c45)c4ccccc34)c2)cc1 ; CC1(C)c2ccccc2-c2c(-c3oc(-c4cccc(-c5ccccc5)c4)c4ccccc34)cccc21]

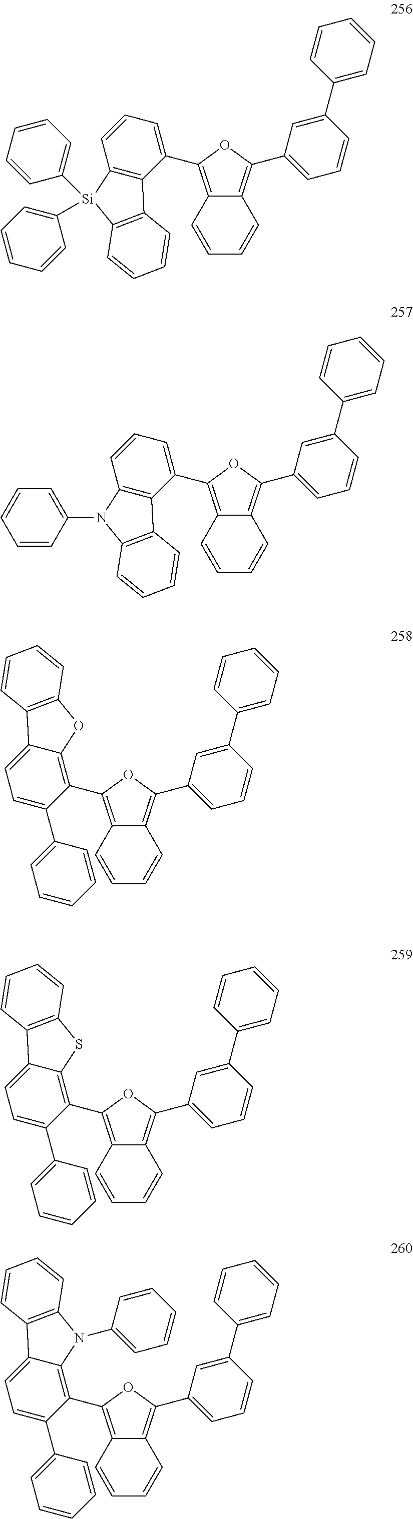

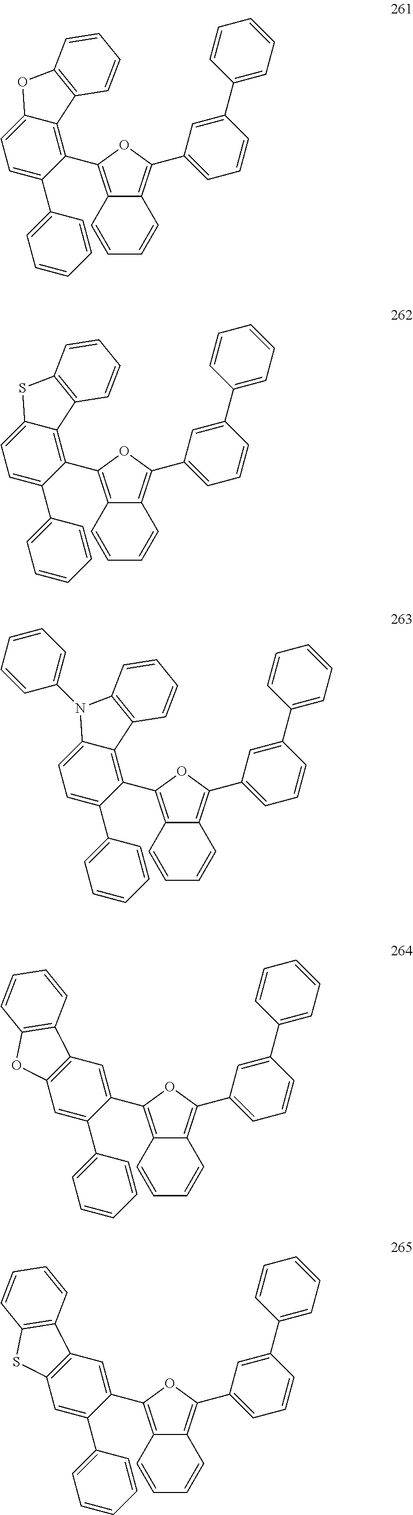 [SMILES: c1ccc(-c2cccc(-c3oc(-c4c(-c5ccccc5)ccc5oc6ccccc6c45)c4ccccc34)c2)cc1 ; c1ccc(-c2cccc(-c3oc(-c4c(-c5ccccc5)ccc5sc6ccccc6c45)c4ccccc34)c2)cc1 ; c1ccc(-c2cccc(-c3oc(-c4c(-c5ccccc5)ccc5c4c4ccccc4n5-c4ccccc4)c4ccccc34)c2)cc1 ; c1ccc(-c2cccc(-c3oc(-c4cc5c(cc4-c4ccccc4)oc4ccccc45)c4ccccc34)c2)cc1 ; c1ccc(-c2cccc(-c3oc(-c4cc5c(cc4-c4ccccc4)sc4ccccc45)c4ccccc34)c2)cc1]

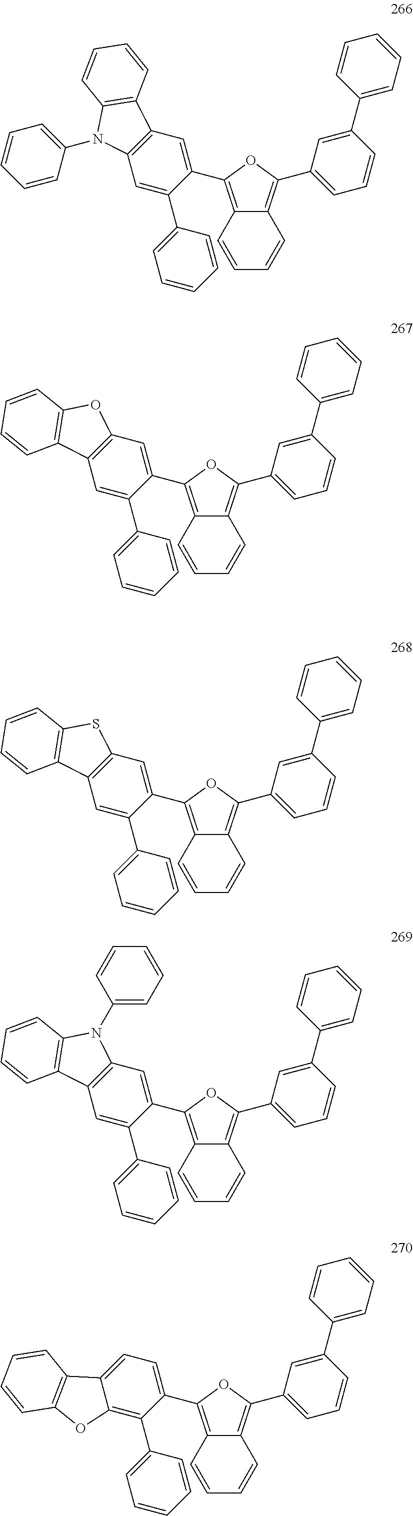

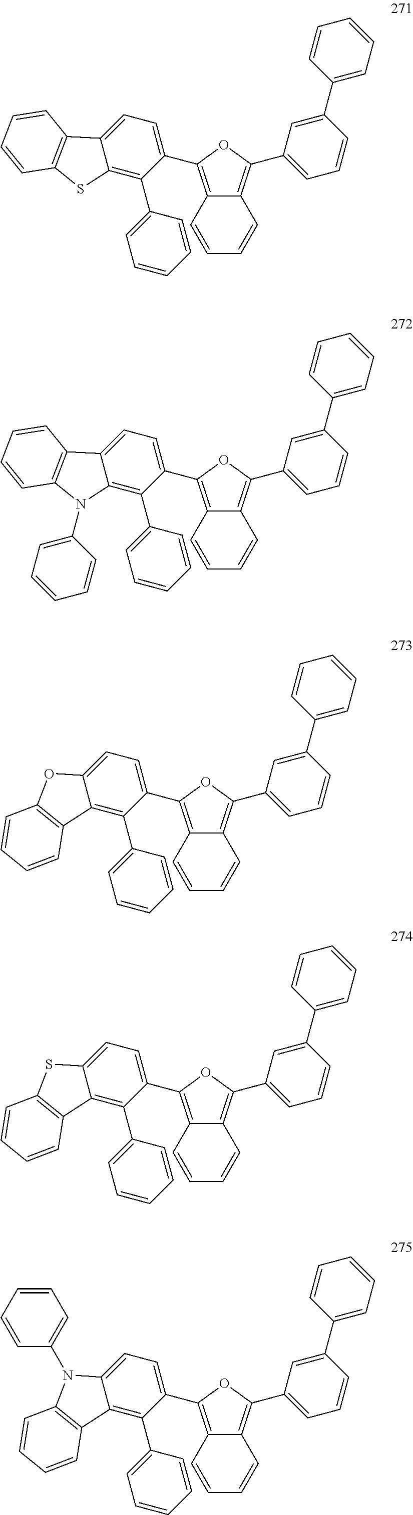 [SMILES: c1ccc(-c2cccc(-c3oc(-c4ccc5c(sc6ccccc65)c4-c4ccccc4)c4ccccc34)c2)cc1 ; c1ccc(-c2cccc(-c3oc(-c4ccc5c6ccccc6n(-c6ccccc6)c5c4-c4ccccc4)c4ccccc34)c2)cc1 ; c1ccc(-c2cccc(-c3oc(-c4ccc5oc6ccccc6c5c4-c4ccccc4)c4ccccc34)c2)cc1 ; c1ccc(-c2cccc(-c3oc(-c4ccc5sc6ccccc6c5c4-c4ccccc4)c4ccccc34)c2)cc1 ; c1ccc(-c2cccc(-c3oc(-c4ccc5c(c4-c4ccccc4)c4ccccc4n5-c4ccccc4)c4ccccc34)c2)cc1]

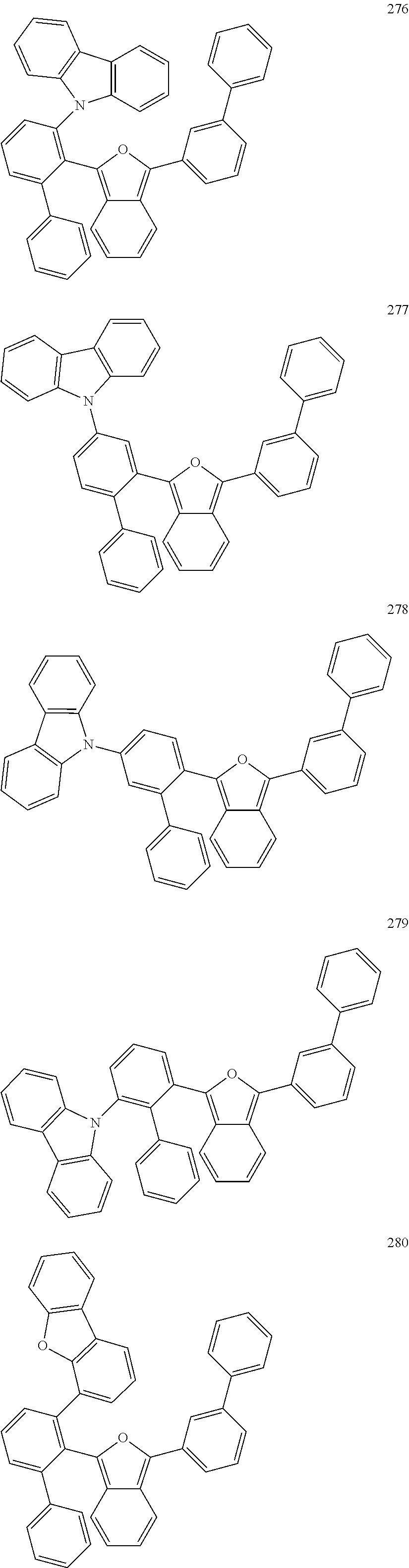

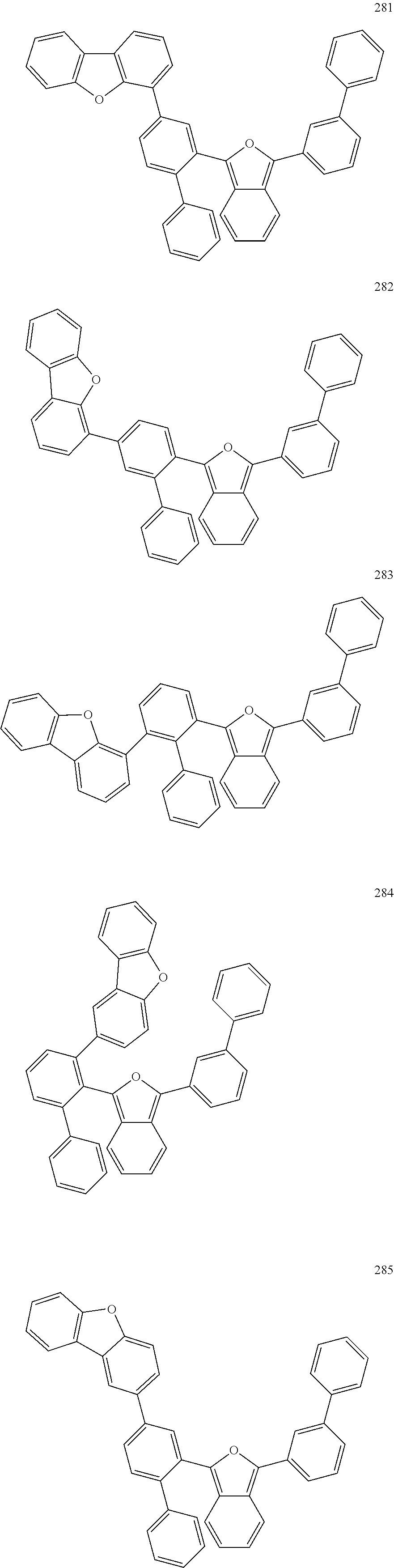 [SMILES: c1ccc(-c2cccc(-c3oc(-c4cc(-c5cccc6c5oc5ccccc56)ccc4-c4ccccc4)c4ccccc34)c2)cc1 ; c1ccc(-c2cccc(-c3oc(-c4ccc(-c5cccc6c5oc5ccccc56)cc4-c4ccccc4)c4ccccc34)c2)cc1 ; c1ccc(-c2cccc(-c3oc(-c4cccc(-c5cccc6c5oc5ccccc56)c4-c4ccccc4)c4ccccc34)c2)cc1 ; c1ccc(-c2cccc(-c3oc(-c4c(-c5ccccc5)cccc4-c4ccc5oc6ccccc6c5c4)c4ccccc34)c2)cc1 ; c1ccc(-c2cccc(-c3oc(-c4cc(-c5ccc6oc7ccccc7c6c5)ccc4-c4ccccc4)c4ccccc34)c2)cc1]

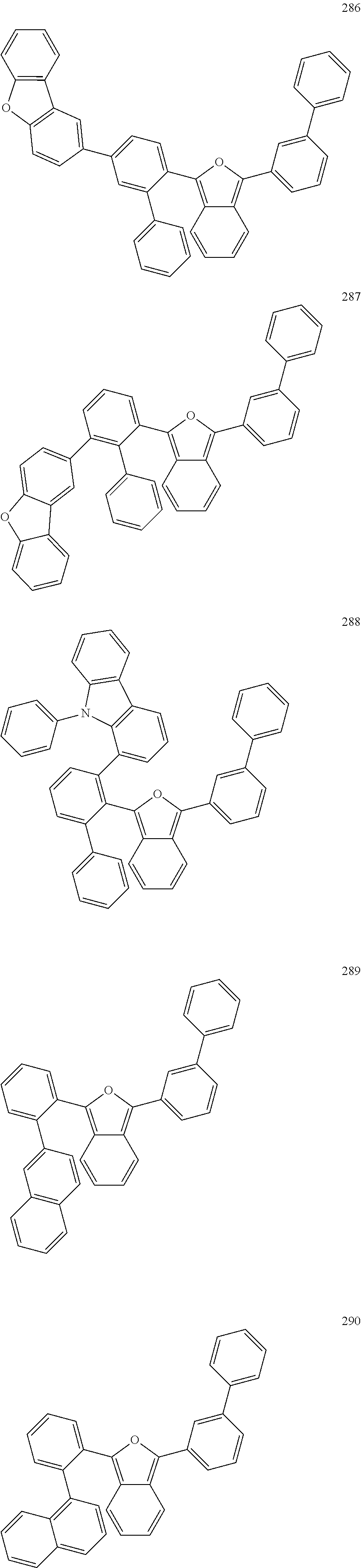 [SMILES: c1ccc(-c2cccc(-c3oc(-c4ccc(-c5ccc6oc7ccccc7c6c5)cc4-c4ccccc4)c4ccccc34)c2)cc1 ; c1ccc(-c2cccc(-c3oc(-c4cccc(-c5ccc6oc7ccccc7c6c5)c4-c4ccccc4)c4ccccc34)c2)cc1 ; c1ccc(-c2cccc(-c3oc(-c4c(-c5ccccc5)cccc4-c4cccc5c6ccccc6n(-c6ccccc6)c45)c4ccccc34)c2)cc1 ; c1ccc(-c2cccc(-c3oc(-c4ccccc4-c4ccc5ccccc5c4)c4ccccc34)c2)cc1 ; c1ccc(-c2cccc(-c3oc(-c4ccccc4-c4cccc5ccccc45)c4ccccc34)c2)cc1]

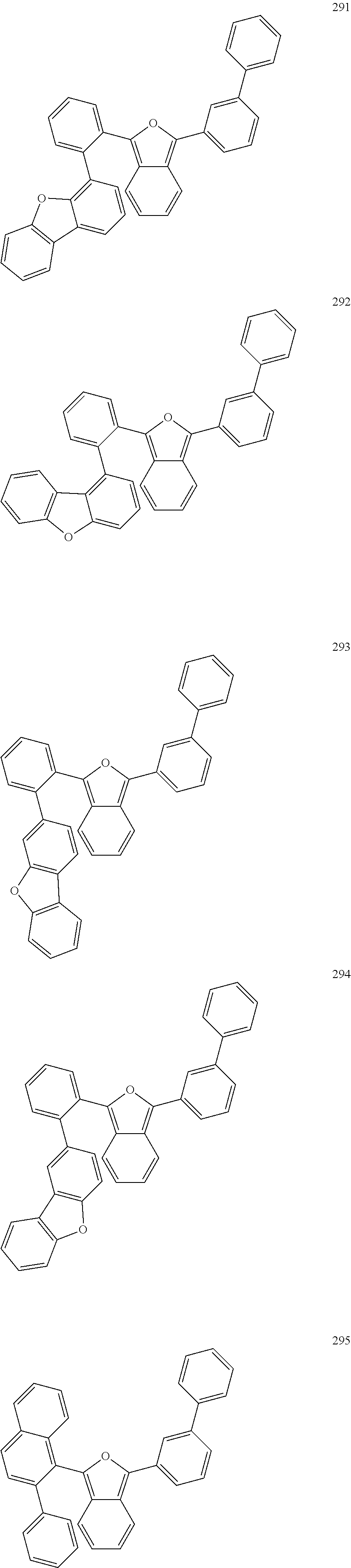 [SMILES: c1ccc(-c2cccc(-c3oc(-c4ccccc4-c4cccc5c4oc4ccccc45)c4ccccc34)c2)cc1 ; c1ccc(-c2cccc(-c3oc(-c4ccccc4-c4cccc5oc6ccccc6c45)c4ccccc34)c2)cc1 ; c1ccc(-c2cccc(-c3oc(-c4ccccc4-c4ccc5c(c4)oc4ccccc45)c4ccccc34)c2)cc1 ; c1ccc(-c2cccc(-c3oc(-c4ccccc4-c4ccc5oc6ccccc6c5c4)c4ccccc34)c2)cc1 ; c1ccc(-c2cccc(-c3oc(-c4c(-c5ccccc5)ccc5ccccc45)c4ccccc34)c2)cc1]

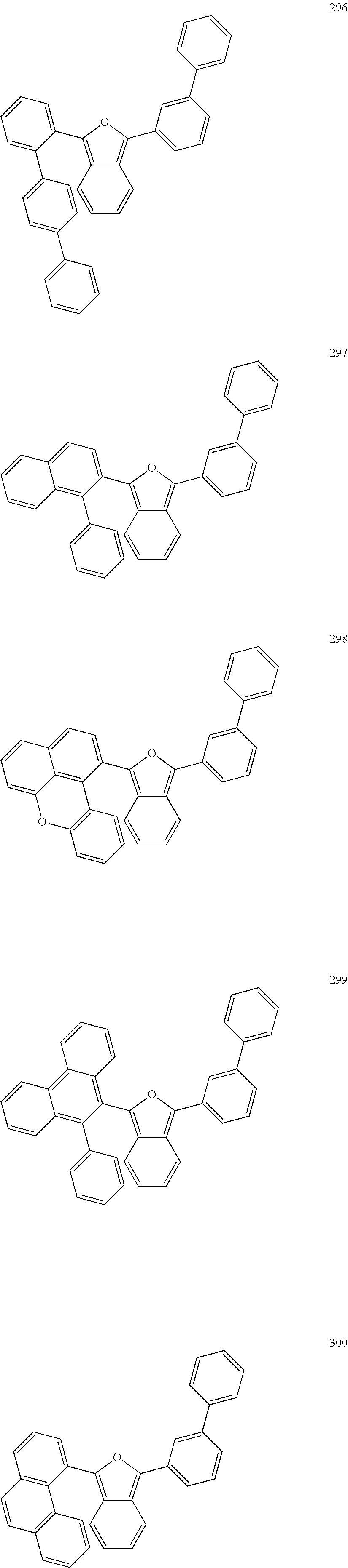 [SMILES: c1ccc(-c2ccc(-c3ccccc3-c3oc(-c4cccc(-c5ccccc5)c4)c4ccccc34)cc2)cc1 ; c1ccc(-c2cccc(-c3oc(-c4ccc5ccccc5c4-c4ccccc4)c4ccccc34)c2)cc1 ; c1ccc(-c2cccc(-c3oc(-c4ccc5cccc6c5c4-c4ccccc4O6)c4ccccc34)c2)cc1 ; c1ccc(-c2cccc(-c3oc(-c4c(-c5ccccc5)c5ccccc5c5ccccc45)c4ccccc34)c2)cc1 ; c1ccc(-c2cccc(-c3oc(-c4cccc5ccc6ccccc6c45)c4ccccc34)c2)cc1]

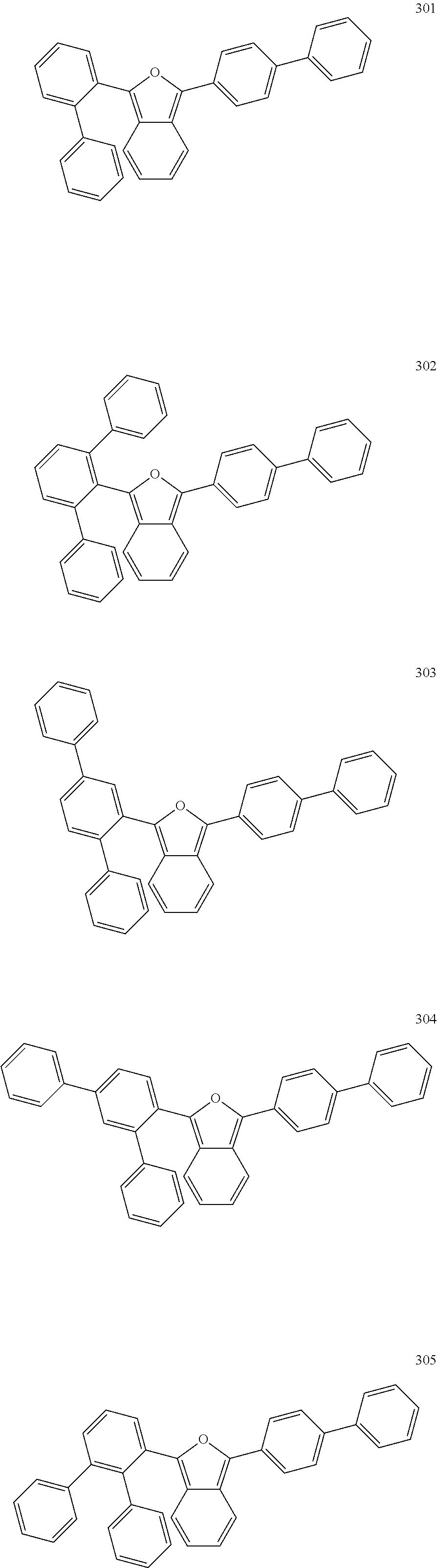 [SMILES: c1ccc(-c2ccc(-c3oc(-c4ccccc4-c4ccccc4)c4ccccc34)cc2)cc1 ; c1ccc(-c2ccc(-c3oc(-c4c(-c5ccccc5)cccc4-c4ccccc4)c4ccccc34)cc2)cc1 ; c1ccc(-c2ccc(-c3oc(-c4cc(-c5ccccc5)ccc4-c4ccccc4)c4ccccc34)cc2)cc1 ; c1ccc(-c2ccc(-c3oc(-c4ccc(-c5ccccc5)cc4-c4ccccc4)c4ccccc34)cc2)cc1 ; c1ccc(-c2ccc(-c3oc(-c4cccc(-c5ccccc5)c4-c4ccccc4)c4ccccc34)cc2)cc1]

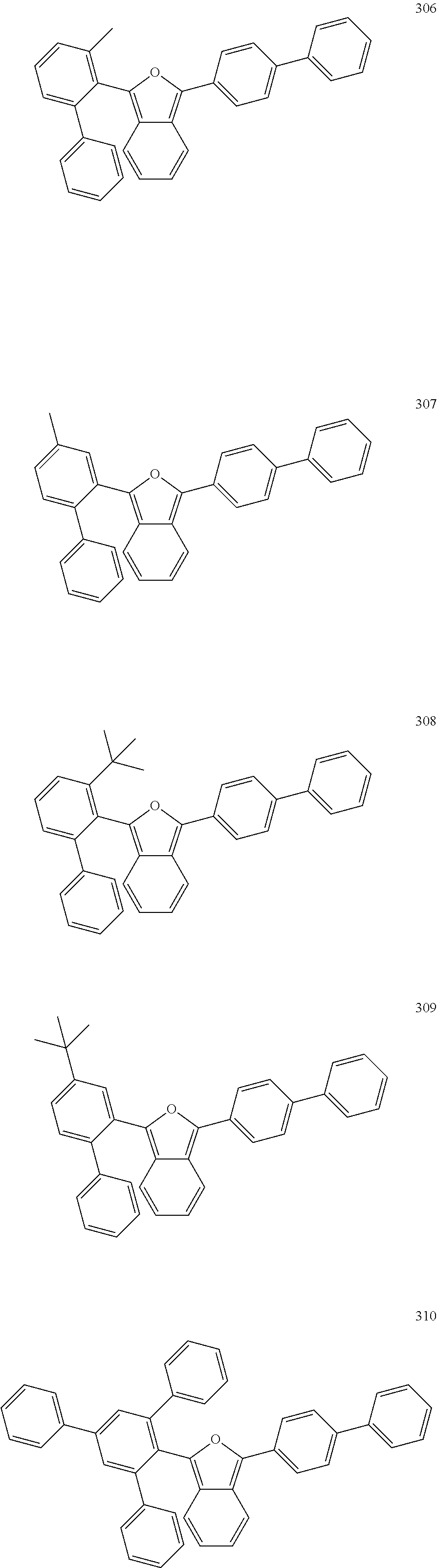 [SMILES: Cc1cccc(-c2ccccc2)c1-c1oc(-c2ccc(-c3ccccc3)cc2)c2ccccc12 ; Cc1ccc(-c2ccccc2)c(-c2oc(-c3ccc(-c4ccccc4)cc3)c3ccccc23)c1 ; CC(C)(C)c1cccc(-c2ccccc2)c1-c1oc(-c2ccc(-c3ccccc3)cc2)c2ccccc12 ; CC(C)(C)c1ccc(-c2ccccc2)c(-c2oc(-c3ccc(-c4ccccc4)cc3)c3ccccc23)c1 ; c1ccc(-c2ccc(-c3oc(-c4c(-c5ccccc5)cc(-c5ccccc5)cc4-c4ccccc4)c4ccccc34)cc2)cc1]

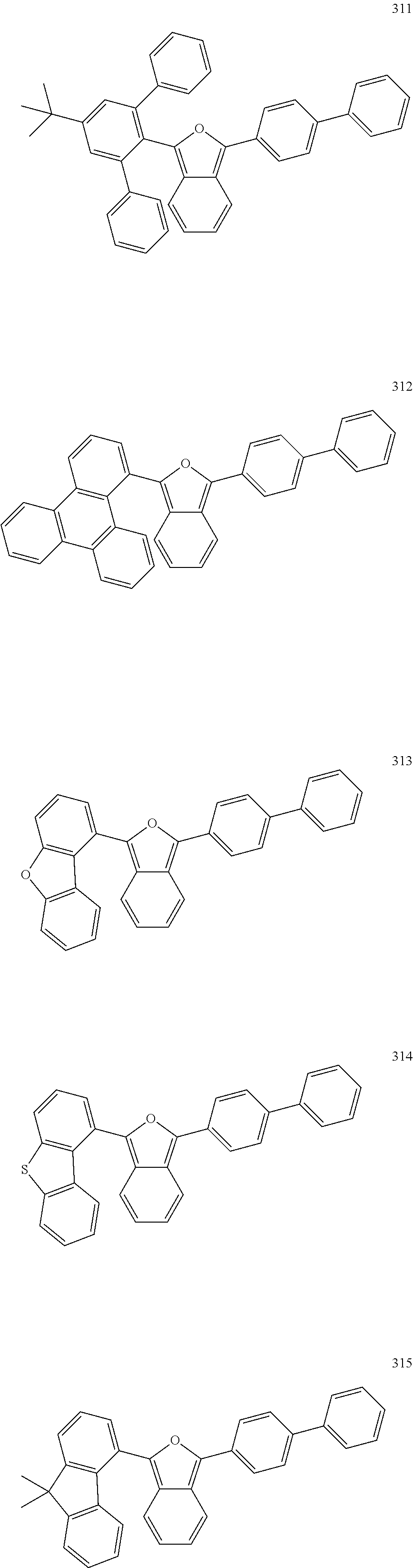 [SMILES: CC(C)(C)c1cc(-c2ccccc2)c(-c2oc(-c3ccc(-c4ccccc4)cc3)c3ccccc23)c(-c2ccccc2)c1 ; c1ccc(-c2ccc(-c3oc(-c4cccc5c6ccccc6c6ccccc6c45)c4ccccc34)cc2)cc1 ; c1ccc(-c2ccc(-c3oc(-c4cccc5oc6ccccc6c45)c4ccccc34)cc2)cc1 ; c1ccc(-c2ccc(-c3oc(-c4cccc5sc6ccccc6c45)c4ccccc34)cc2)cc1 ; CC1(C)c2ccccc2-c2c(-c3oc(-c4ccc(-c5ccccc5)cc4)c4ccccc34)cccc21]

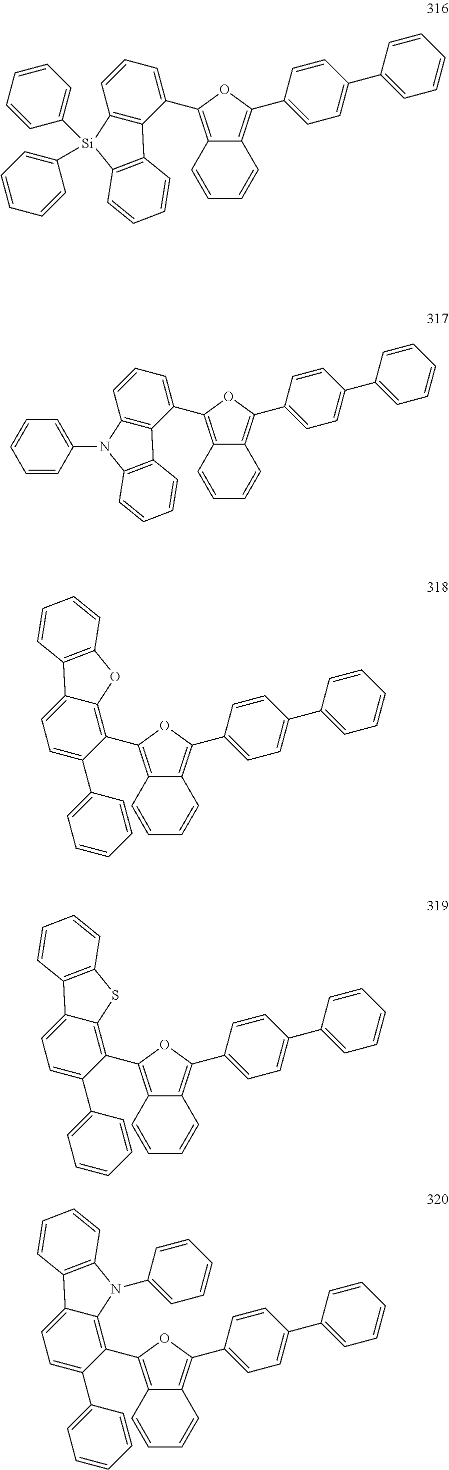

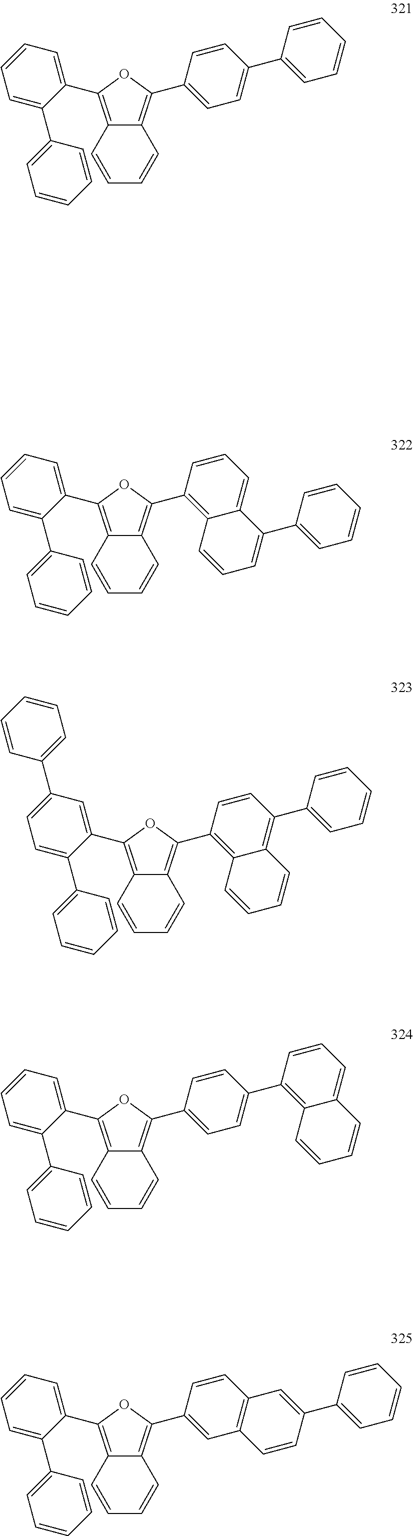 [SMILES: c1ccc(-c2ccc(-c3oc(-c4ccccc4-c4ccccc4)c4ccccc34)cc2)cc1 ; c1ccc(-c2ccccc2-c2oc(-c3cccc4c(-c5ccccc5)cccc34)c3ccccc23)cc1 ; c1ccc(-c2ccc(-c3ccccc3)c(-c3oc(-c4ccc(-c5ccccc5)c5ccccc45)c4ccccc34)c2)cc1 ; c1ccc(-c2ccccc2-c2oc(-c3ccc(-c4cccc5ccccc45)cc3)c3ccccc23)cc1 ; c1ccc(-c2ccc3cc(-c4oc(-c5ccccc5-c5ccccc5)c5ccccc45)ccc3c2)cc1]

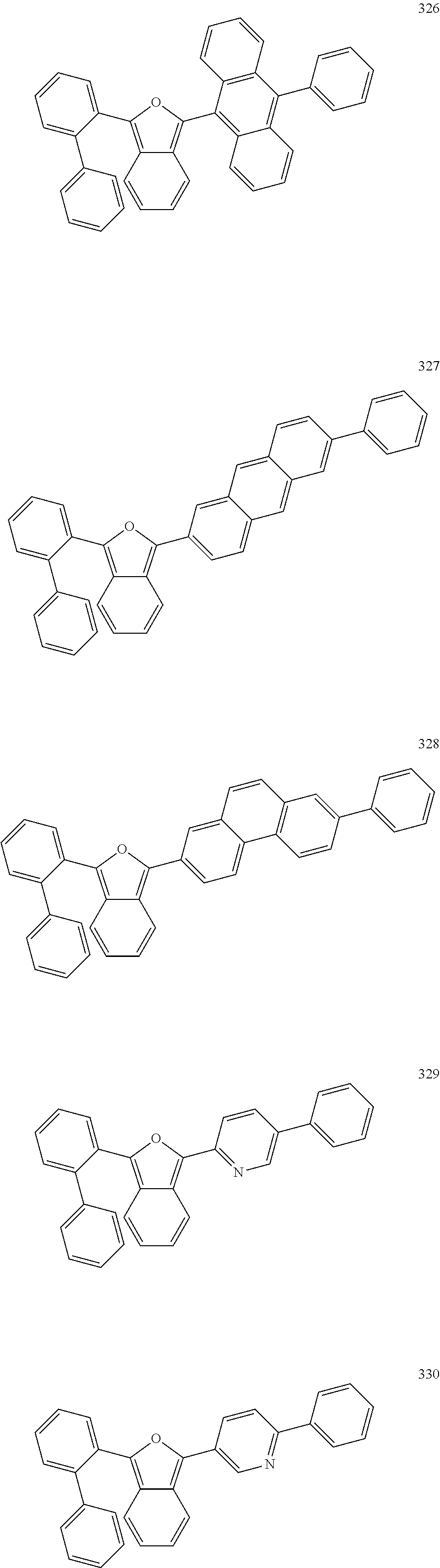 [SMILES: c1ccc(-c2ccccc2-c2oc(-c3c4ccccc4c(-c4ccccc4)c4ccccc34)c3ccccc23)cc1 ; c1ccc(-c2ccc3cc4cc(-c5oc(-c6ccccc6-c6ccccc6)c6ccccc56)ccc4cc3c2)cc1 ; c1ccc(-c2ccc3c(ccc4cc(-c5oc(-c6ccccc6-c6ccccc6)c6ccccc56)ccc43)c2)cc1 ; c1ccc(-c2ccc(-c3oc(-c4ccccc4-c4ccccc4)c4ccccc34)nc2)cc1 ; c1ccc(-c2ccc(-c3oc(-c4ccccc4-c4ccccc4)c4ccccc34)cn2)cc1]

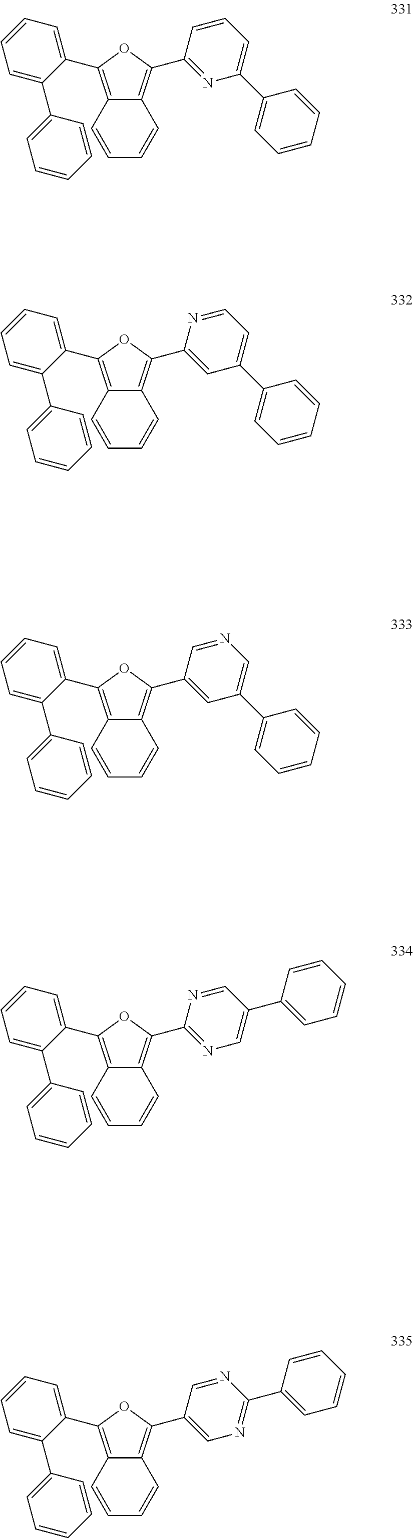 [SMILES: c1ccc(-c2cccc(-c3oc(-c4ccccc4-c4ccccc4)c4ccccc34)n2)cc1 ; c1ccc(-c2ccnc(-c3oc(-c4ccccc4-c4ccccc4)c4ccccc34)c2)cc1 ; c1ccc(-c2cncc(-c3oc(-c4ccccc4-c4ccccc4)c4ccccc34)c2)cc1 ; c1ccc(-c2cnc(-c3oc(-c4ccccc4-c4ccccc4)c4ccccc34)nc2)cc1 ; c1ccc(-c2ncc(-c3oc(-c4ccccc4-c4ccccc4)c4ccccc34)cn2)cc1]

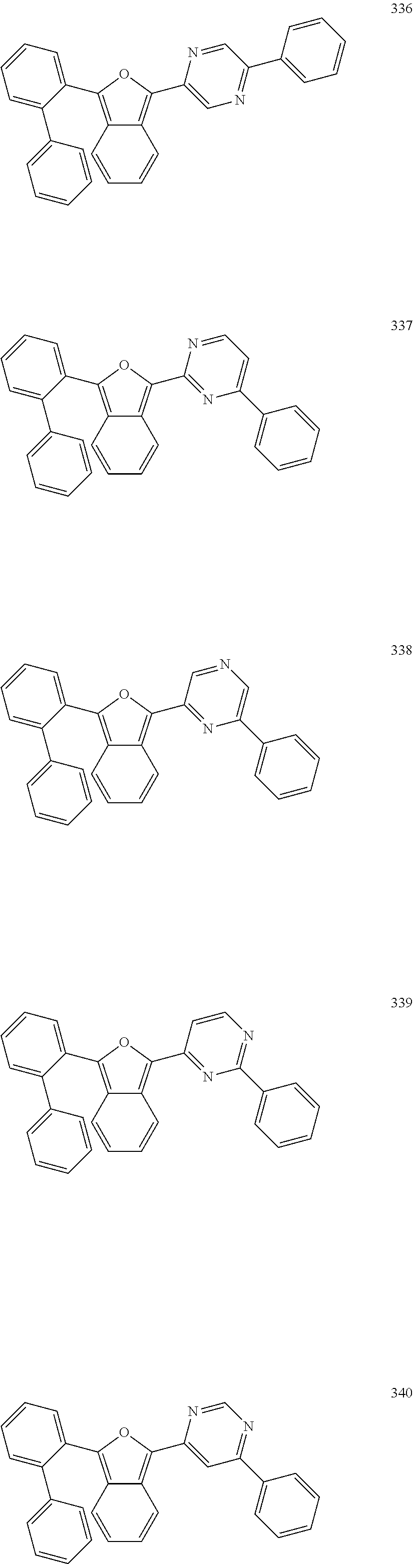 [SMILES: c1ccc(-c2cnc(-c3oc(-c4ccccc4-c4ccccc4)c4ccccc34)cn2)cc1 ; c1ccc(-c2ccnc(-c3oc(-c4ccccc4-c4ccccc4)c4ccccc34)n2)cc1 ; c1ccc(-c2cncc(-c3oc(-c4ccccc4-c4ccccc4)c4ccccc34)n2)cc1 ; c1ccc(-c2nccc(-c3oc(-c4ccccc4-c4ccccc4)c4ccccc34)n2)cc1 ; c1ccc(-c2cc(-c3oc(-c4ccccc4-c4ccccc4)c4ccccc34)ncn2)cc1]

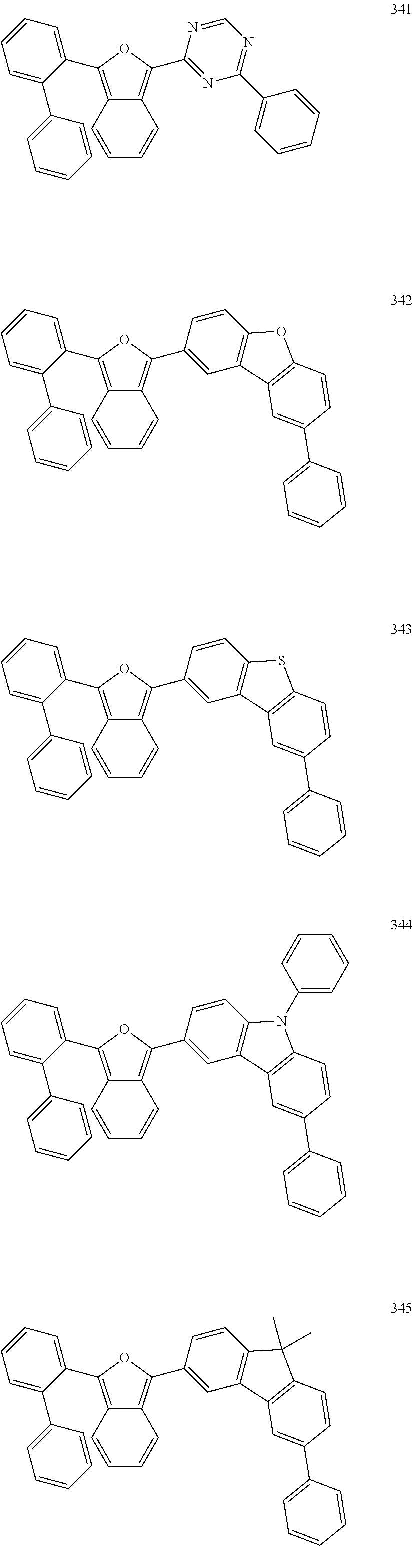 [SMILES: c1ccc(-c2ncnc(-c3oc(-c4ccccc4-c4ccccc4)c4ccccc34)n2)cc1 ; c1ccc(-c2ccc3oc4ccc(-c5oc(-c6ccccc6-c6ccccc6)c6ccccc56)cc4c3c2)cc1 ; c1ccc(-c2ccc3sc4ccc(-c5oc(-c6ccccc6-c6ccccc6)c6ccccc56)cc4c3c2)cc1 ; c1ccc(-c2ccc3c(c2)c2cc(-c4oc(-c5ccccc5-c5ccccc5)c5ccccc45)ccc2n3-c2ccccc2)cc1 ; CC1(C)c2ccc(-c3ccccc3)cc2-c2cc(-c3oc(-c4ccccc4-c4ccccc4)c4ccccc34)ccc21]

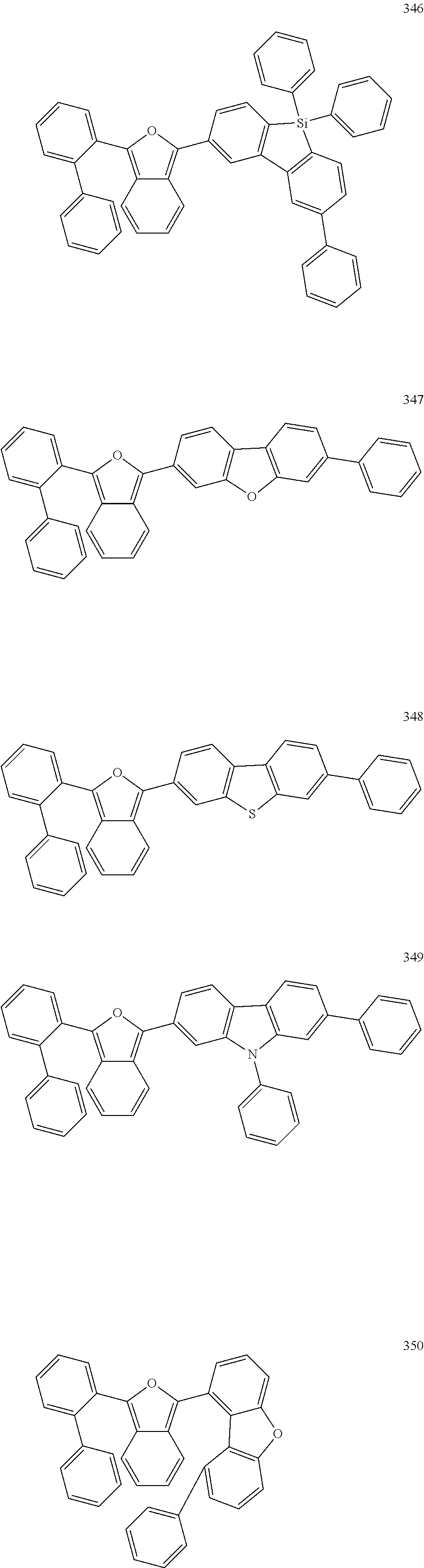 [SMILES: c1ccc(-c2ccc3c(c2)-c2cc(-c4oc(-c5ccccc5-c5ccccc5)c5ccccc45)ccc2[Si]3(c2ccccc2)c2ccccc2)cc1 ; c1ccc(-c2ccc3c(c2)oc2cc(-c4oc(-c5ccccc5-c5ccccc5)c5ccccc45)ccc23)cc1 ; c1ccc(-c2ccc3c(c2)sc2cc(-c4oc(-c5ccccc5-c5ccccc5)c5ccccc45)ccc23)cc1 ; c1ccc(-c2ccc3c4ccc(-c5oc(-c6ccccc6-c6ccccc6)c6ccccc56)cc4n(-c4ccccc4)c3c2)cc1 ; c1ccc(-c2ccccc2-c2oc(-c3cccc4oc5cccc(-c6ccccc6)c5c34)c3ccccc23)cc1]

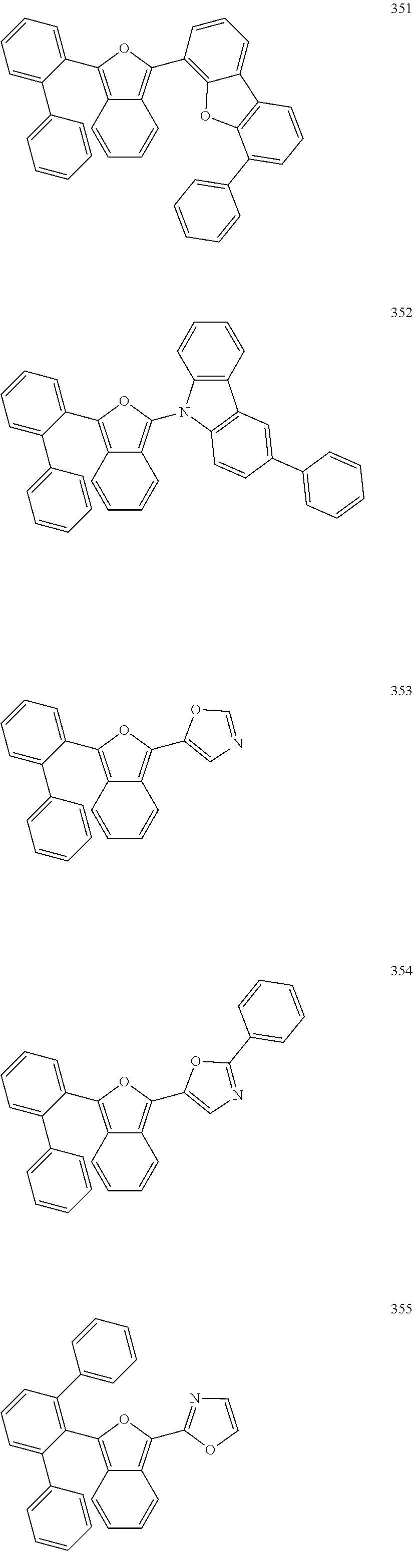 [SMILES: c1ccc(-c2ccccc2-c2oc(-c3cccc4c3oc3c(-c5ccccc5)cccc34)c3ccccc23)cc1 ; c1ccc(-c2ccc3c(c2)c2ccccc2n3-c2oc(-c3ccccc3-c3ccccc3)c3ccccc23)cc1 ; c1ccc(-c2ccccc2-c2oc(-c3cnco3)c3ccccc23)cc1 ; c1ccc(-c2ncc(-c3oc(-c4ccccc4-c4ccccc4)c4ccccc34)o2)cc1 ; c1ccc(-c2cccc(-c3ccccc3)c2-c2oc(-c3ncco3)c3ccccc23)cc1]

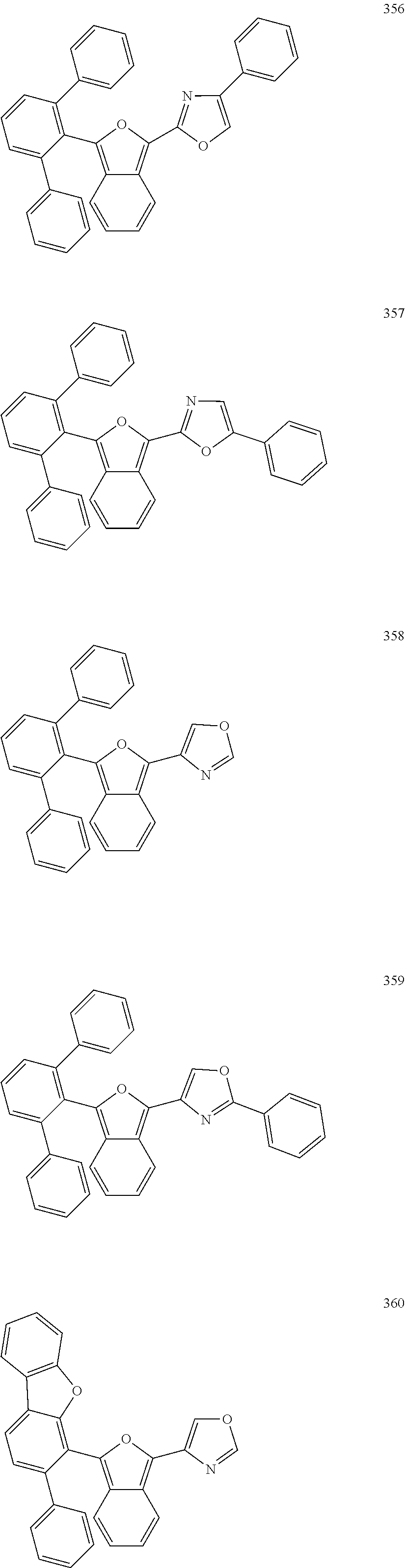 [SMILES: c1ccc(-c2coc(-c3oc(-c4c(-c5ccccc5)cccc4-c4ccccc4)c4ccccc34)n2)cc1 ; c1ccc(-c2cnc(-c3oc(-c4c(-c5ccccc5)cccc4-c4ccccc4)c4ccccc34)o2)cc1 ; c1ccc(-c2cccc(-c3ccccc3)c2-c2oc(-c3cocn3)c3ccccc23)cc1 ; c1ccc(-c2nc(-c3oc(-c4c(-c5ccccc5)cccc4-c4ccccc4)c4ccccc34)co2)cc1 ; c1ccc(-c2ccc3c(oc4ccccc43)c2-c2oc(-c3cocn3)c3ccccc23)cc1]

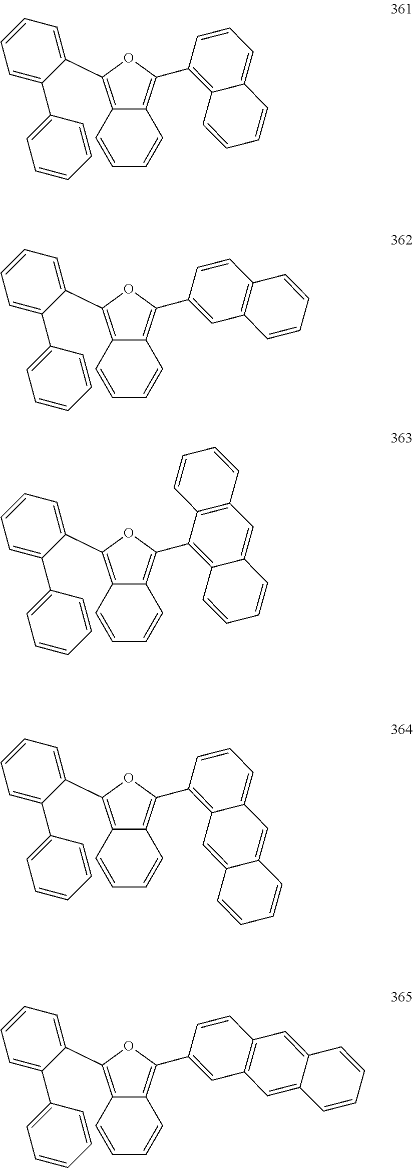 [SMILES: c1ccc(-c2ccccc2-c2oc(-c3cccc4ccccc34)c3ccccc23)cc1 ; c1ccc(-c2ccccc2-c2oc(-c3ccc4ccccc4c3)c3ccccc23)cc1 ; c1ccc(-c2ccccc2-c2oc(-c3c4ccccc4cc4ccccc34)c3ccccc23)cc1 ; c1ccc(-c2ccccc2-c2oc(-c3cccc4cc5ccccc5cc34)c3ccccc23)cc1 ; c1ccc(-c2ccccc2-c2oc(-c3ccc4cc5ccccc5cc4c3)c3ccccc23)cc1]

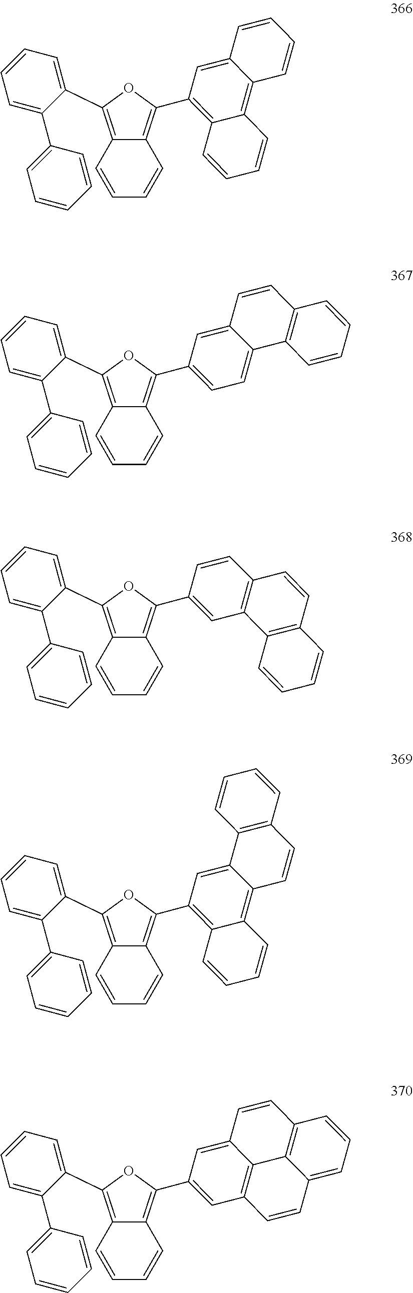 [SMILES: c1ccc(-c2ccccc2-c2oc(-c3cc4ccccc4c4ccccc34)c3ccccc23)cc1 ; c1ccc(-c2ccccc2-c2oc(-c3ccc4c(ccc5ccccc54)c3)c3ccccc23)cc1 ; c1ccc(-c2ccccc2-c2oc(-c3ccc4ccc5ccccc5c4c3)c3ccccc23)cc1 ; c1ccc(-c2ccccc2-c2oc(-c3cc4c5ccccc5ccc4c4ccccc34)c3ccccc23)cc1 ; c1ccc(-c2ccccc2-c2oc(-c3cc4ccc5cccc6ccc(c3)c4c56)c3ccccc23)cc1]

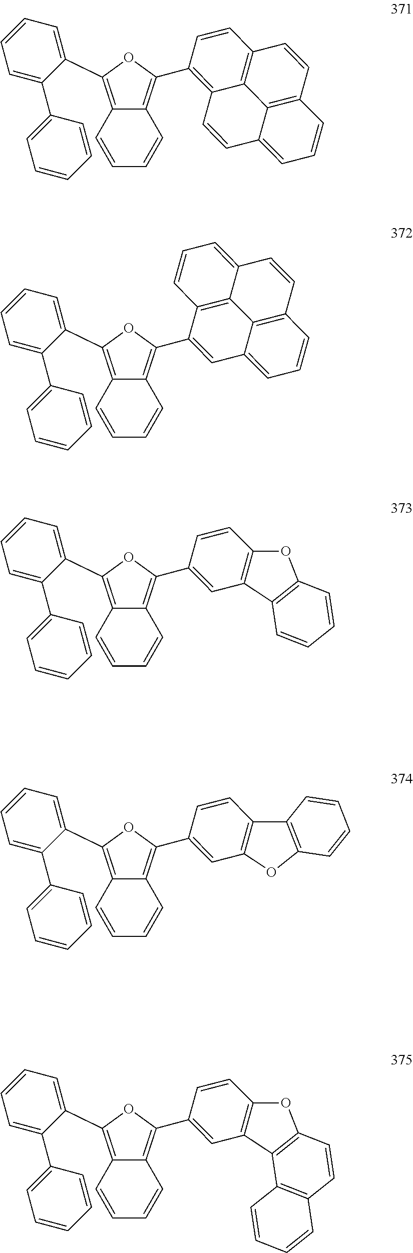 [SMILES: c1ccc(-c2ccccc2-c2oc(-c3ccc4ccc5cccc6ccc3c4c56)c3ccccc23)cc1 ; c1ccc(-c2ccccc2-c2oc(-c3cc4cccc5ccc6cccc3c6c54)c3ccccc23)cc1 ; c1ccc(-c2ccccc2-c2oc(-c3ccc4oc5ccccc5c4c3)c3ccccc23)cc1 ; c1ccc(-c2ccccc2-c2oc(-c3ccc4c(c3)oc3ccccc34)c3ccccc23)cc1 ; c1ccc(-c2ccccc2-c2oc(-c3ccc4oc5ccc6ccccc6c5c4c3)c3ccccc23)cc1]

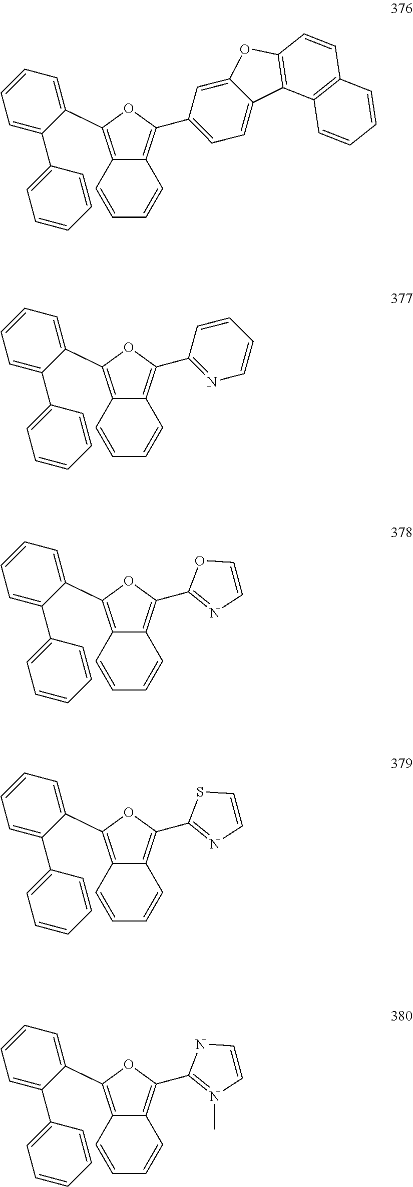

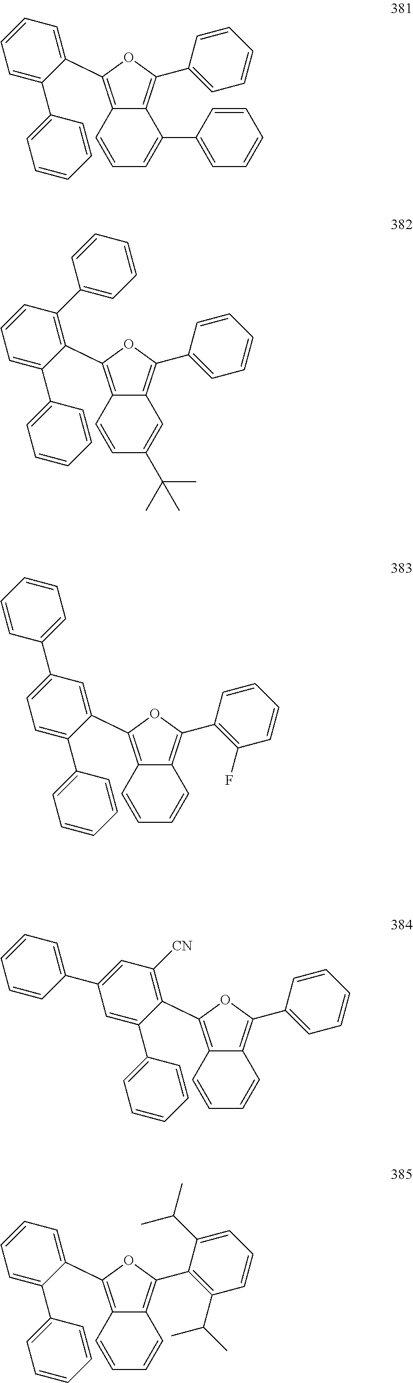 [SMILES: c1ccc(-c2ccccc2-c2oc(-c3ccccc3)c3c(-c4ccccc4)cccc23)cc1 ; CC(C)(C)c1ccc2c(-c3c(-c4ccccc4)cccc3-c3ccccc3)oc(-c3ccccc3)c2c1 ; Fc1ccccc1-c1oc(-c2cc(-c3ccccc3)ccc2-c2ccccc2)c2ccccc12 ; N#Cc1cc(-c2ccccc2)cc(-c2ccccc2)c1-c1oc(-c2ccccc2)c2ccccc12 ; CC(C)c1cccc(C(C)C)c1-c1oc(-c2ccccc2-c2ccccc2)c2ccccc12]

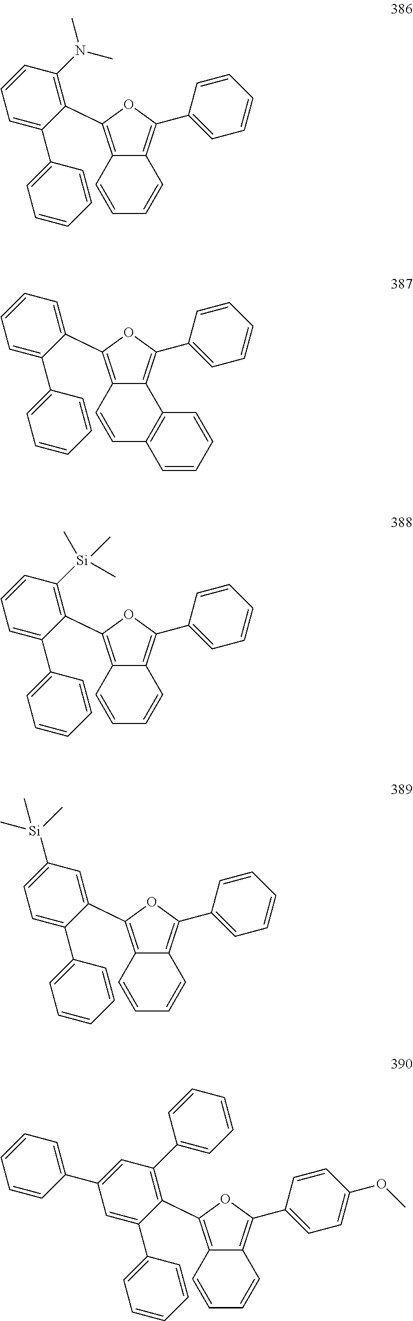 [SMILES: CN(C)c1cccc(-c2ccccc2)c1-c1oc(-c2ccccc2)c2ccccc12 ; c1ccc(-c2ccccc2-c2oc(-c3ccccc3)c3c2ccc2ccccc23)cc1 ; C[Si](C)(C)c1cccc(-c2ccccc2)c1-c1oc(-c2ccccc2)c2ccccc12 ; C[Si](C)(C)c1ccc(-c2ccccc2)c(-c2oc(-c3ccccc3)c3ccccc23)c1 ; COc1ccc(-c2oc(-c3c(-c4ccccc4)cc(-c4ccccc4)cc3-c3ccccc3)c3ccccc23)cc1]

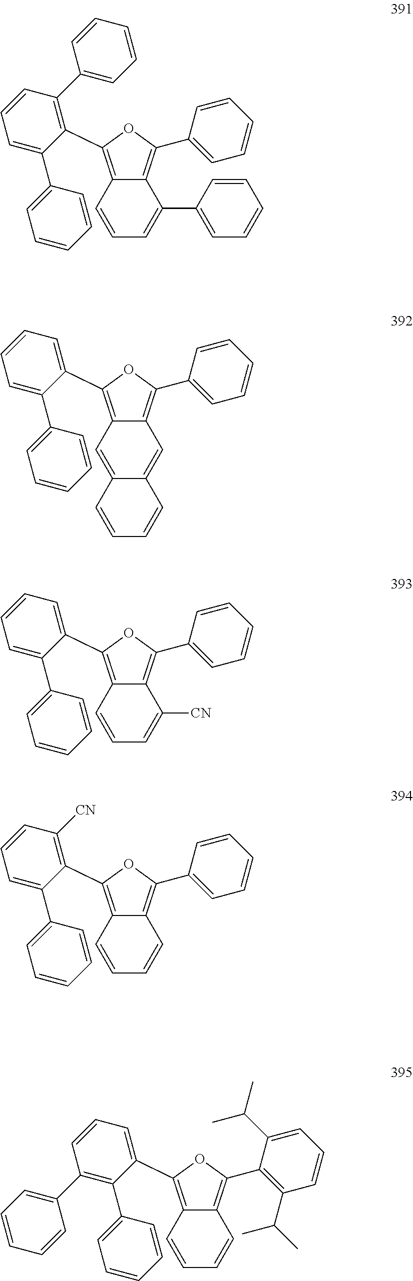 [SMILES: c1ccc(-c2cccc(-c3ccccc3)c2-c2oc(-c3ccccc3)c3c(-c4ccccc4)cccc23)cc1 ; c1ccc(-c2ccccc2-c2oc(-c3ccccc3)c3cc4ccccc4cc23)cc1 ; N#Cc1cccc2c(-c3ccccc3-c3ccccc3)oc(-c3ccccc3)c12 ; N#Cc1cccc(-c2ccccc2)c1-c1oc(-c2ccccc2)c2ccccc12 ; CC(C)c1cccc(C(C)C)c1-c1oc(-c2cccc(-c3ccccc3)c2-c2ccccc2)c2ccccc12]

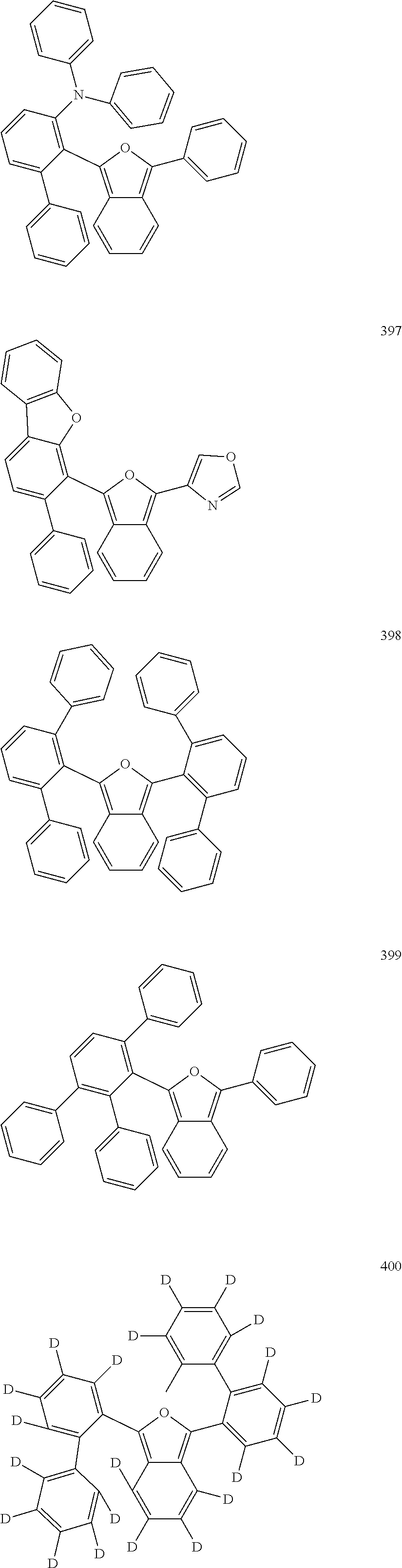 [SMILES: c1ccc(-c2cccc(N(c3ccccc3)c3ccccc3)c2-c2oc(-c3ccccc3)c3ccccc23)cc1 ; c1ccc(-c2ccc3c(oc4ccccc43)c2-c2oc(-c3cocn3)c3ccccc23)cc1 ; c1ccc(-c2cccc(-c3ccccc3)c2-c2oc(-c3c(-c4ccccc4)cccc3-c3ccccc3)c3ccccc23)cc1 ; c1ccc(-c2ccc(-c3ccccc3)c(-c3oc(-c4ccccc4)c4ccccc34)c2-c2ccccc2)cc1 ; [2H]c1c([2H])c([2H])c(-c2c([2H])c([2H])c([2H])c([2H])c2-c2oc(-c3c([2H])c([2H])c([2H])c([2H])c3-c3c([2H])c([2H])c([2H])c([2H])c3C)c3c([2H])c([2H])c([2H])c([2H])c23)c([2H])c1[2H]]

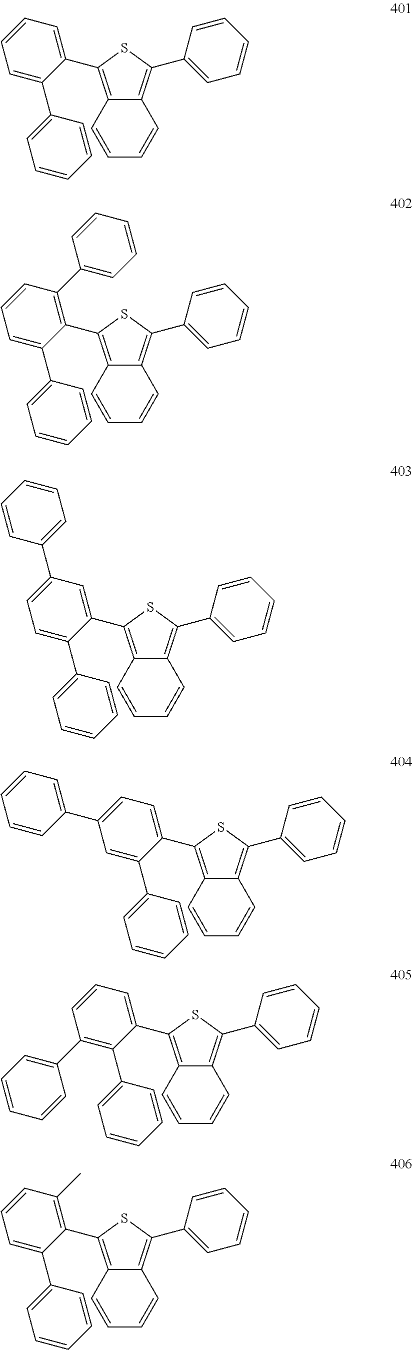

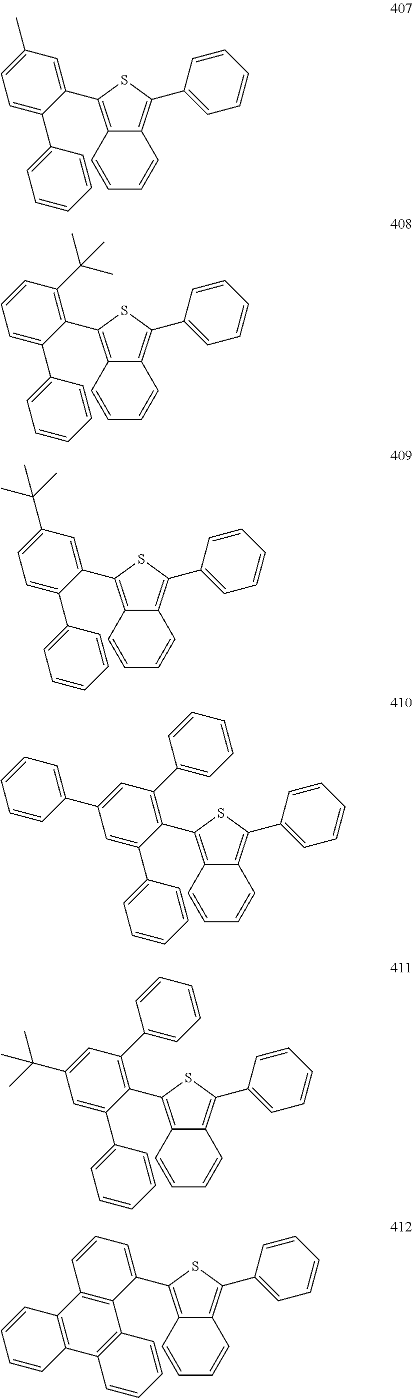

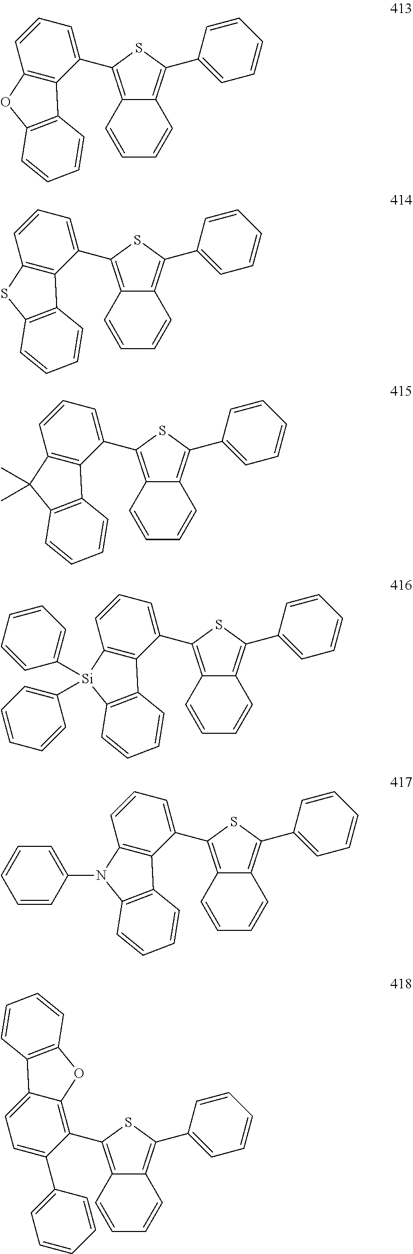

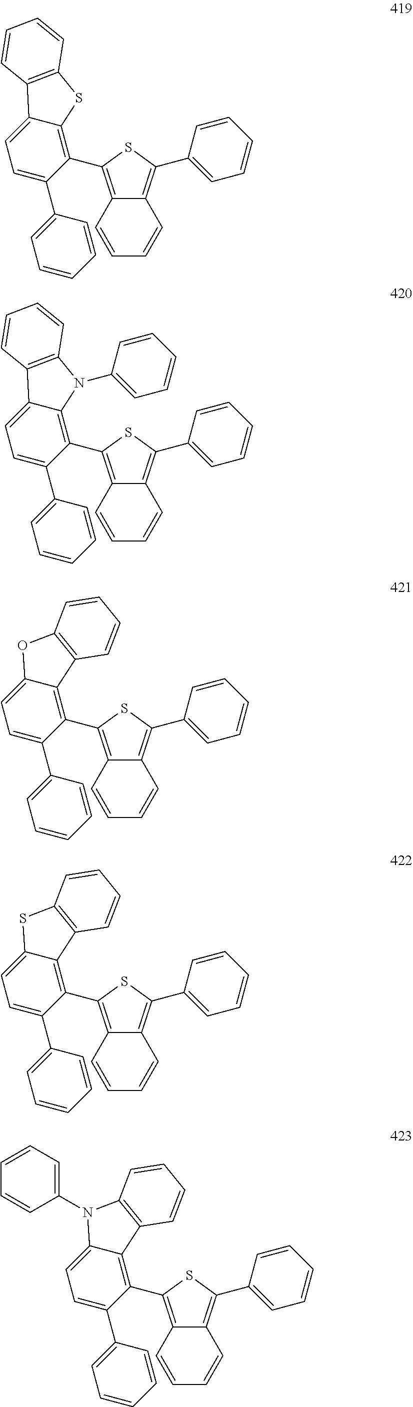

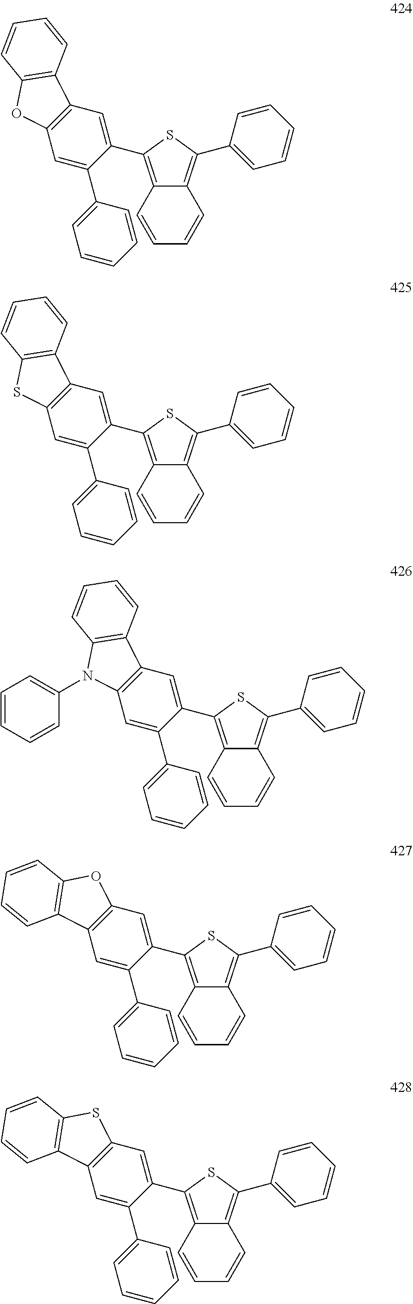

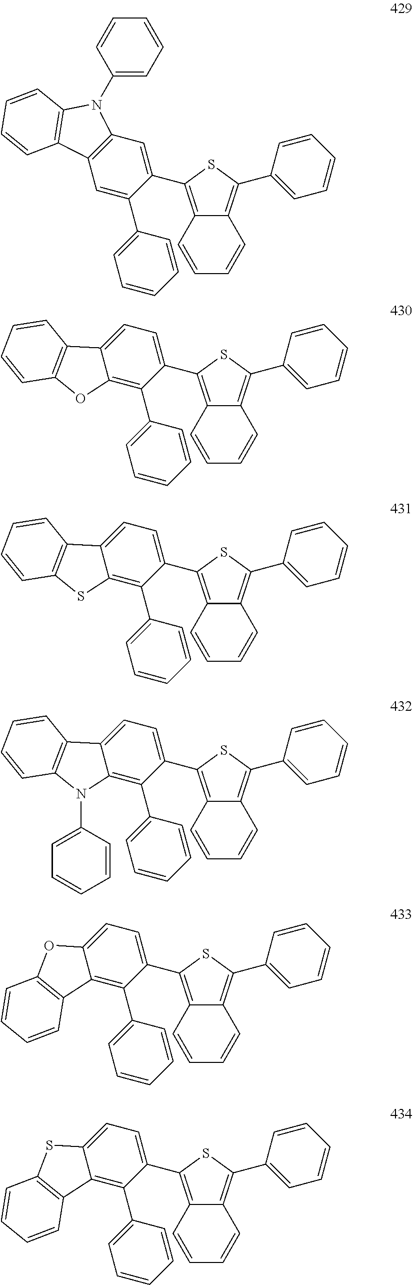

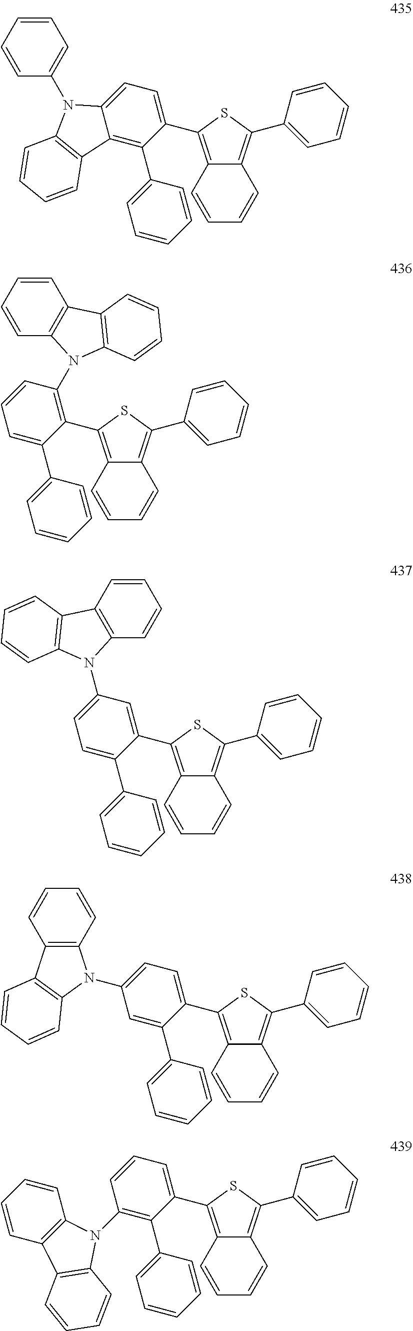

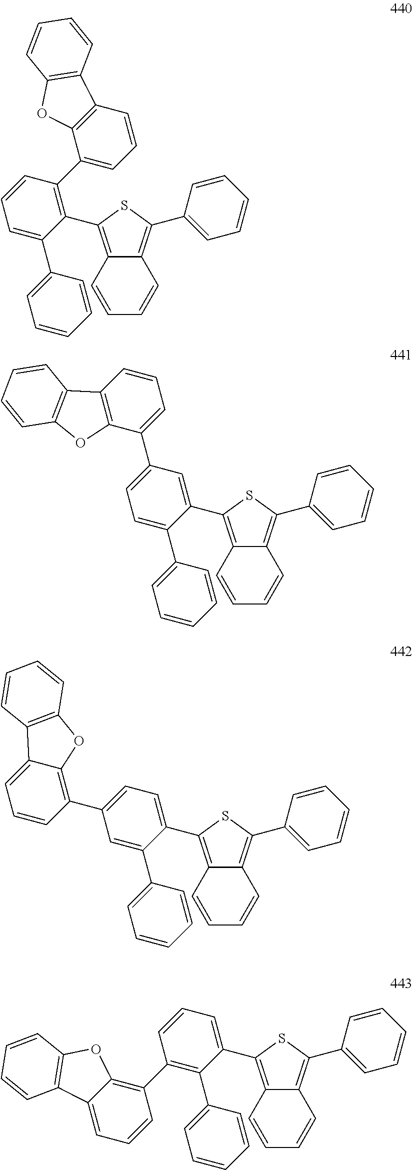

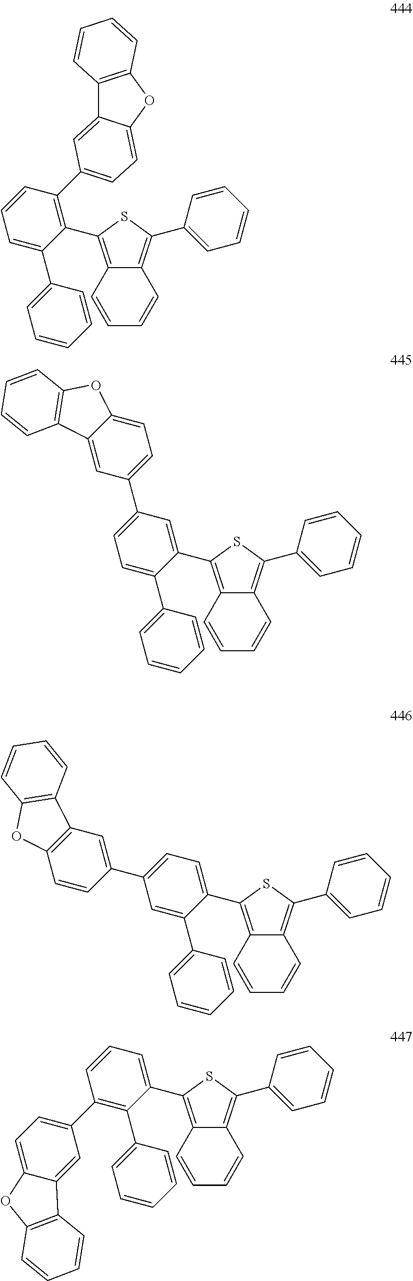

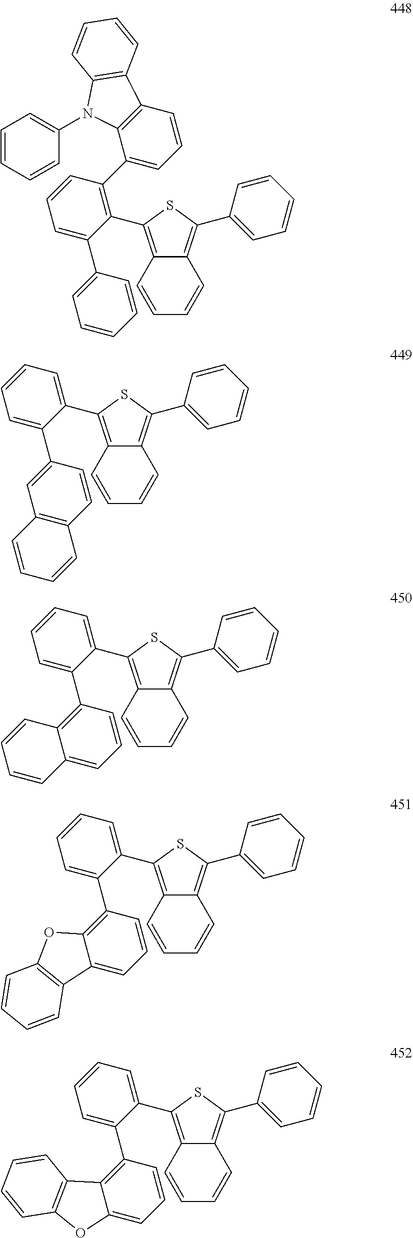

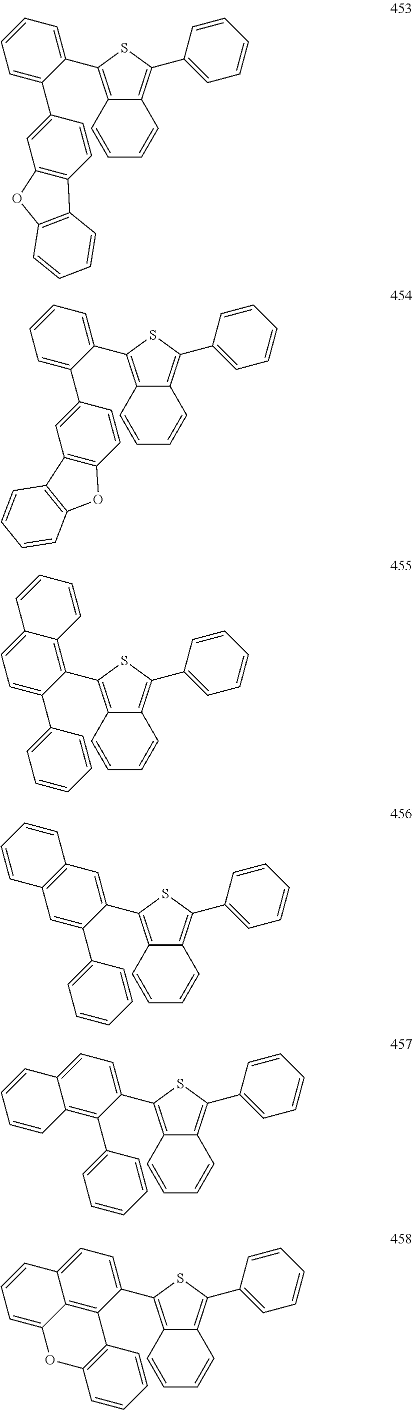

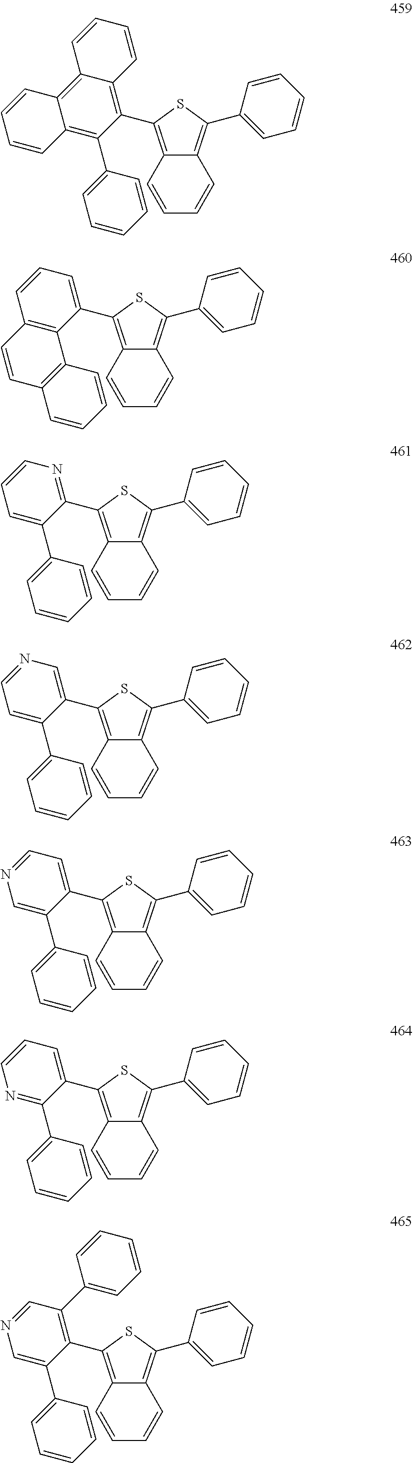

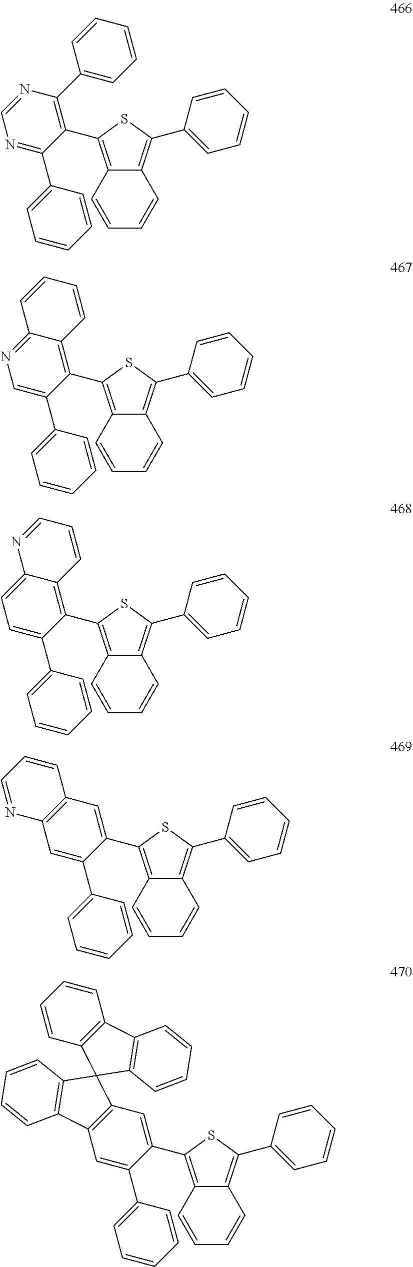

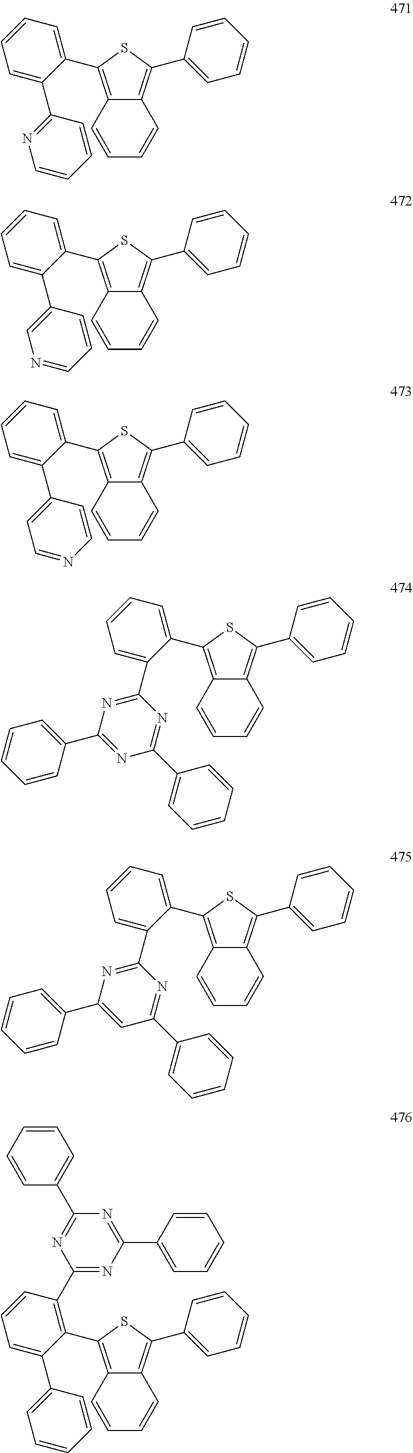

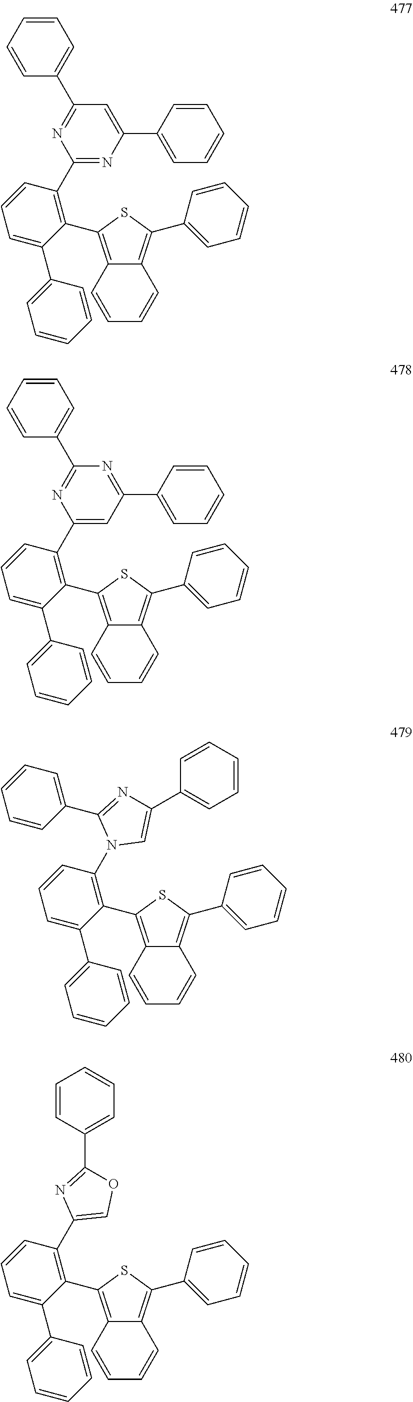

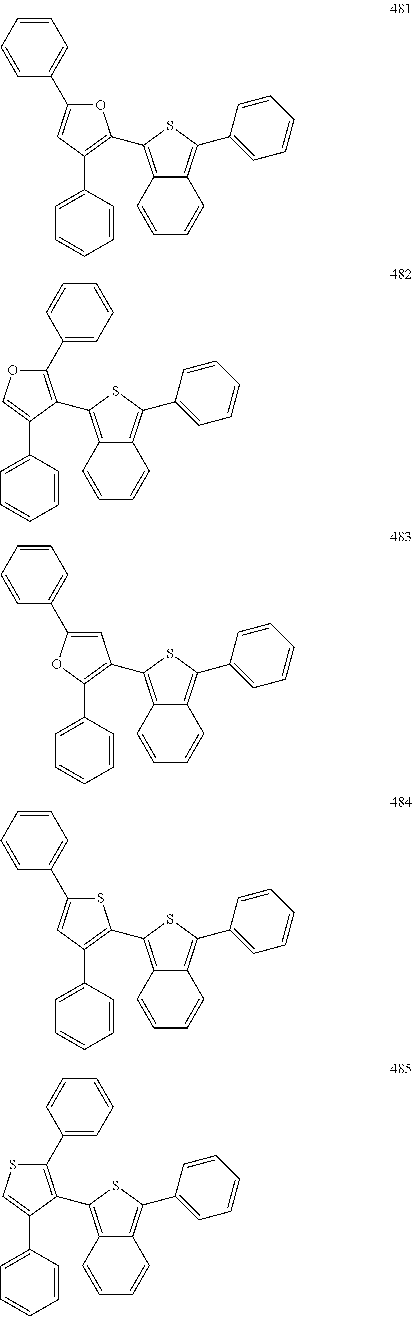

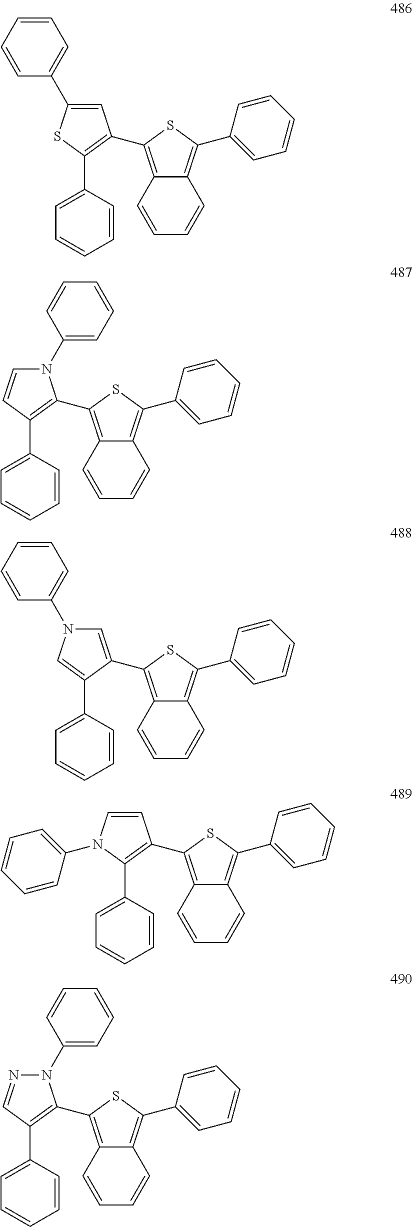

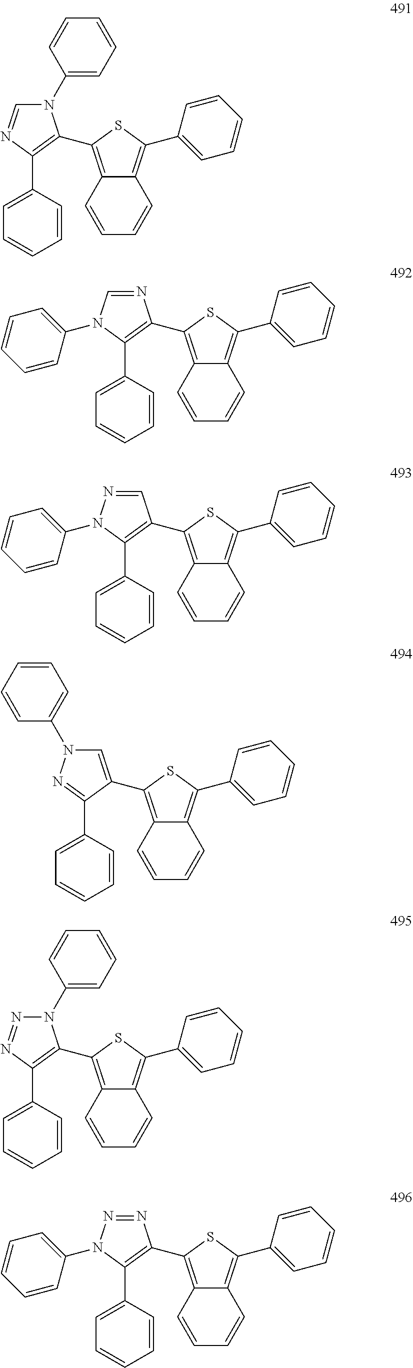

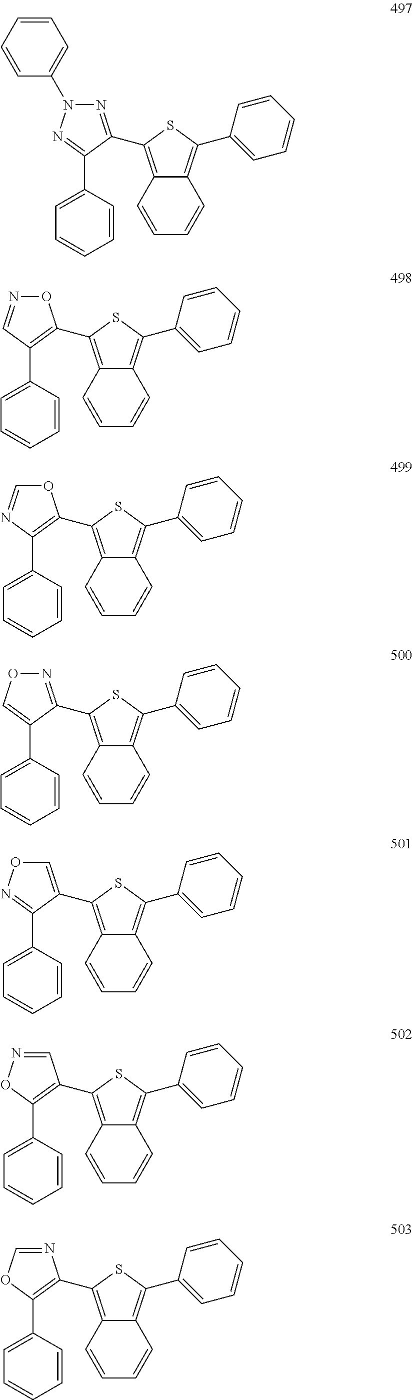

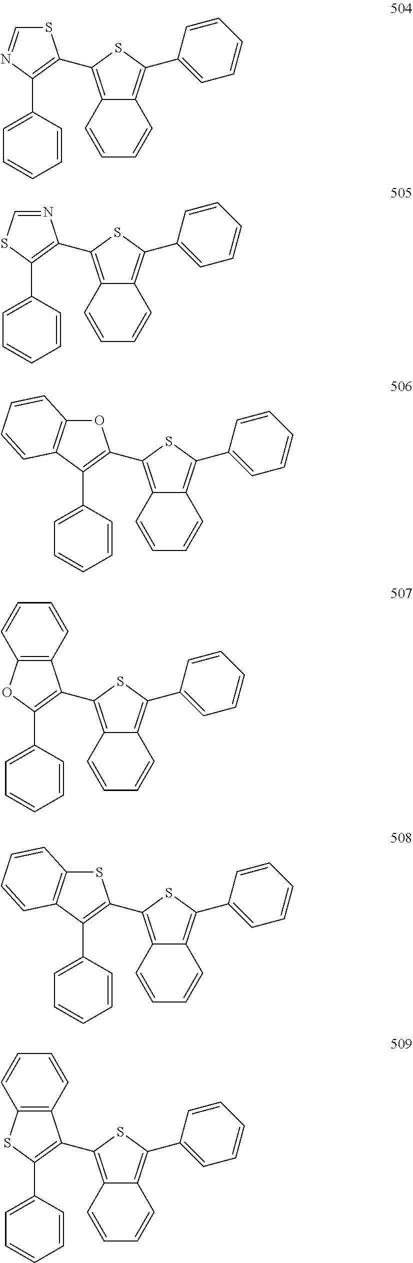

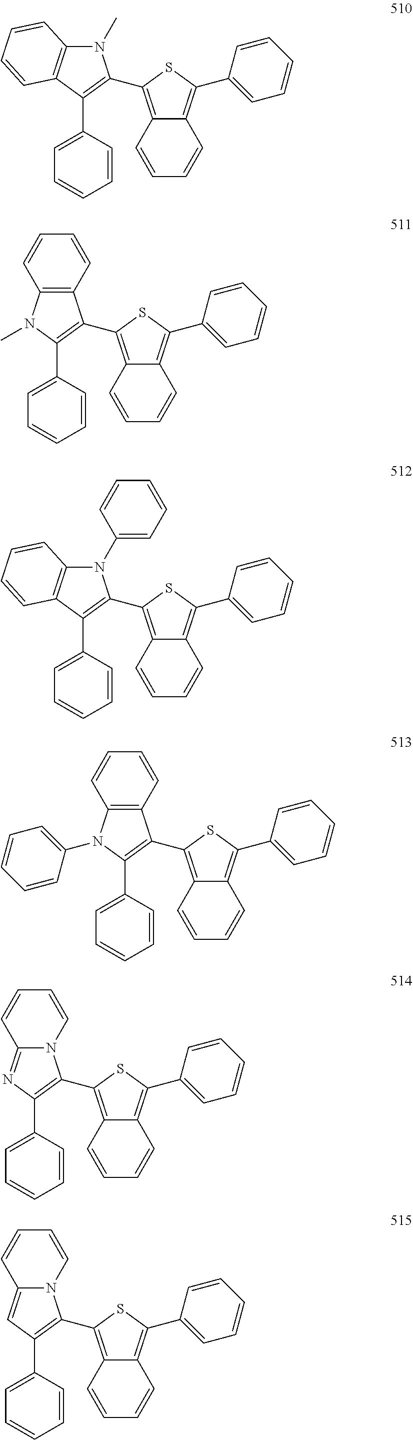

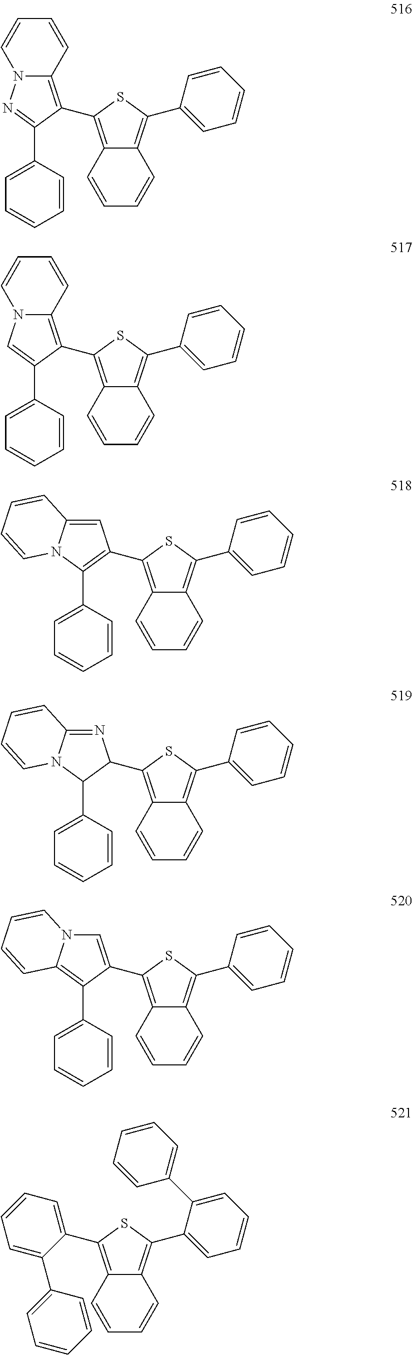

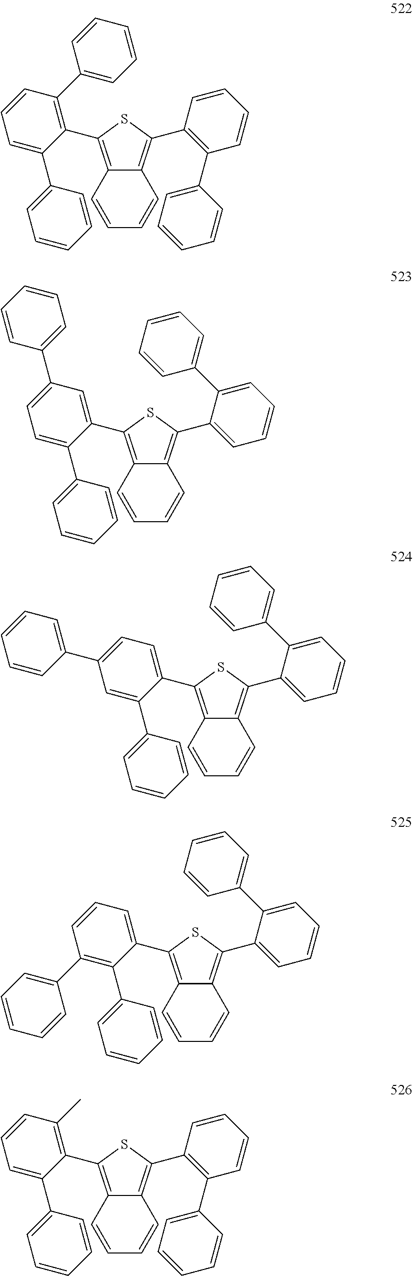

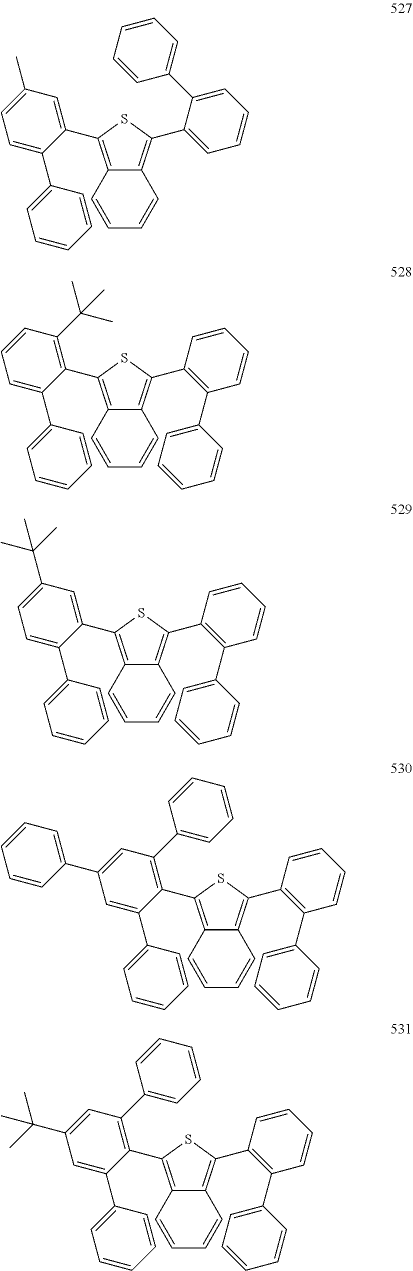

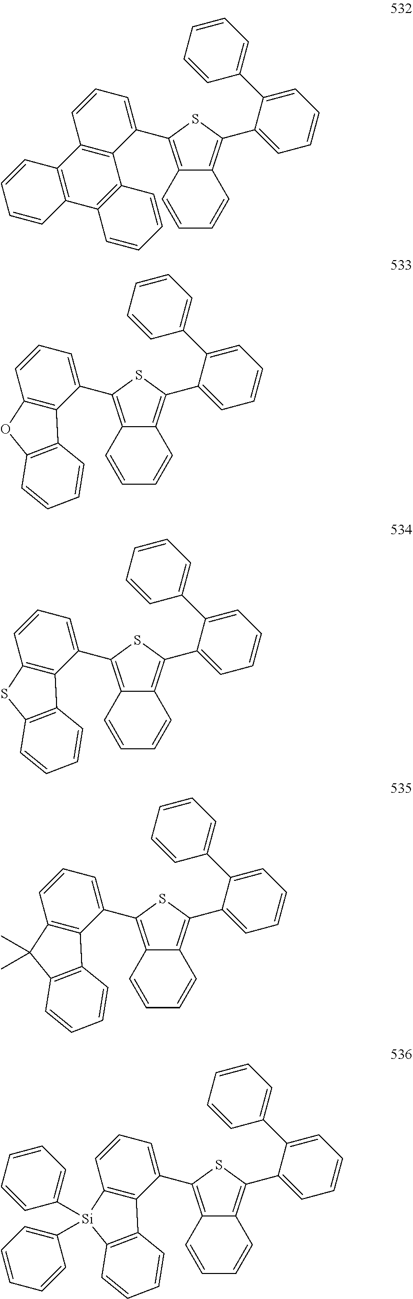

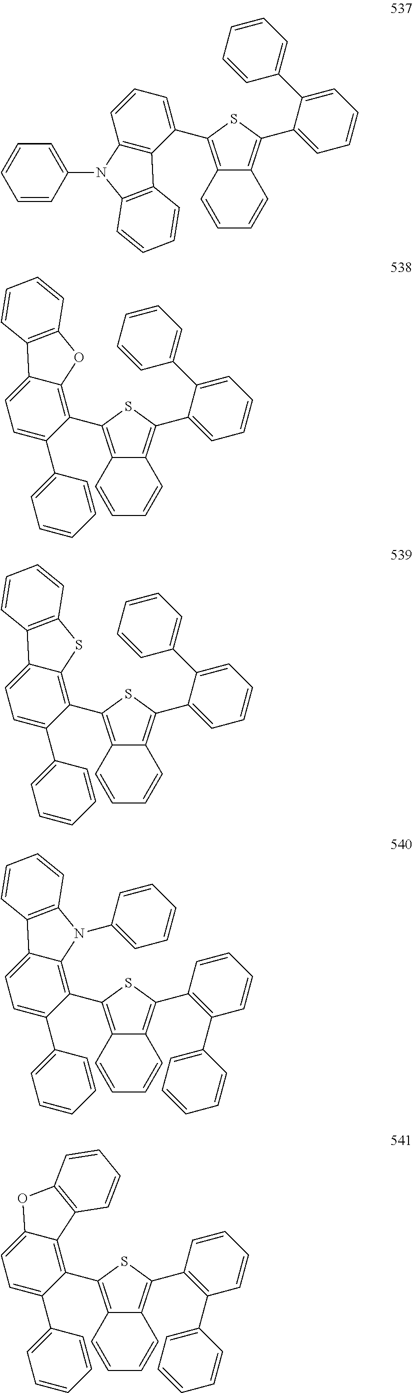

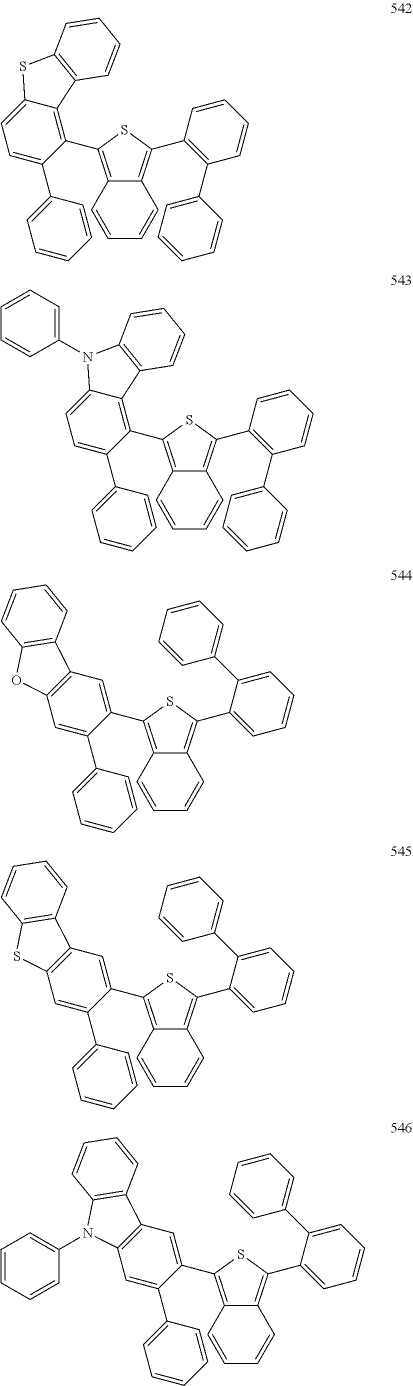

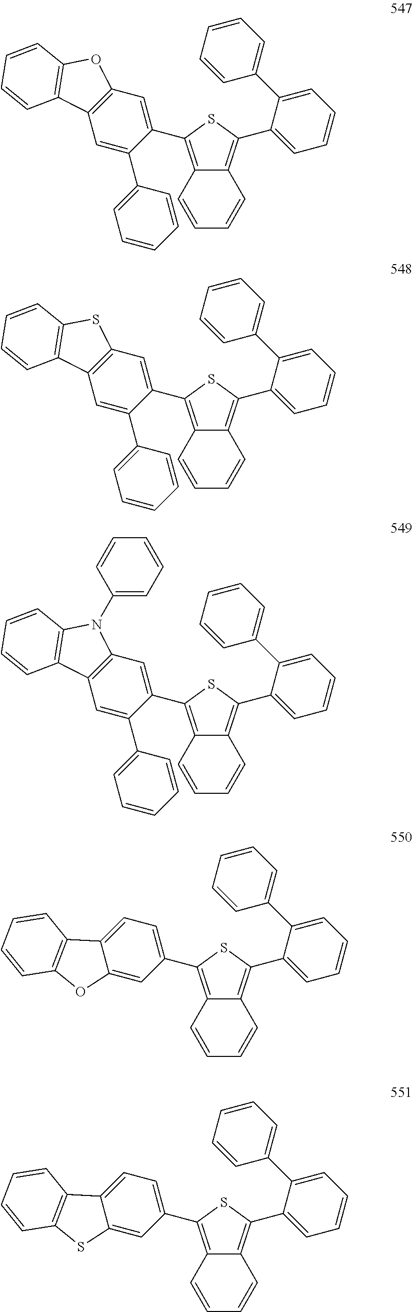

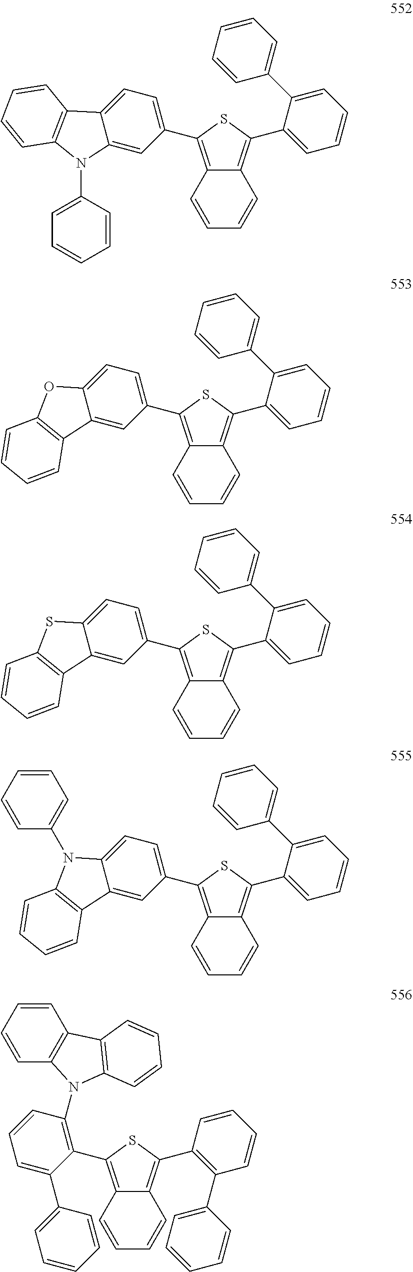

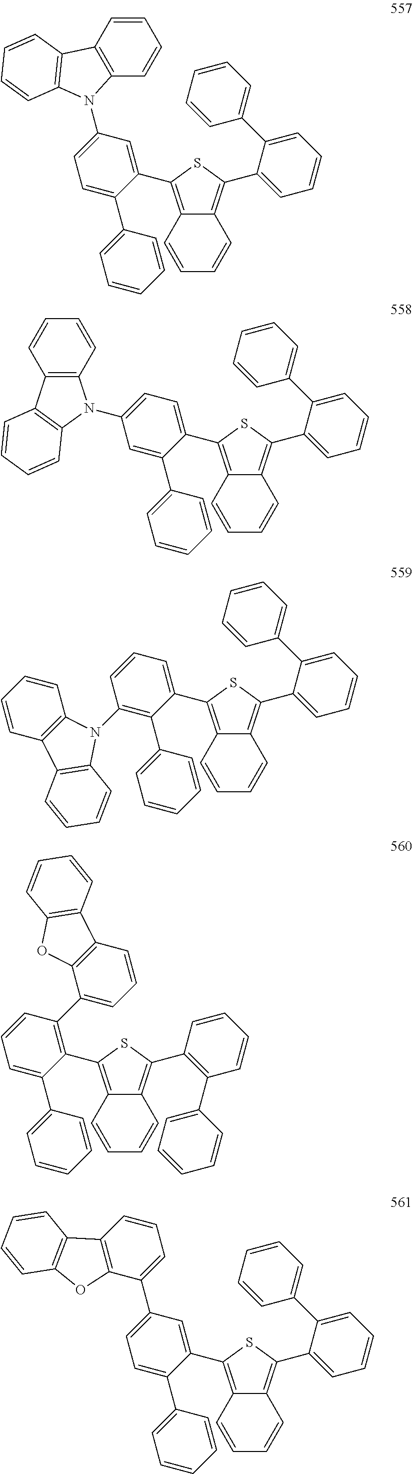

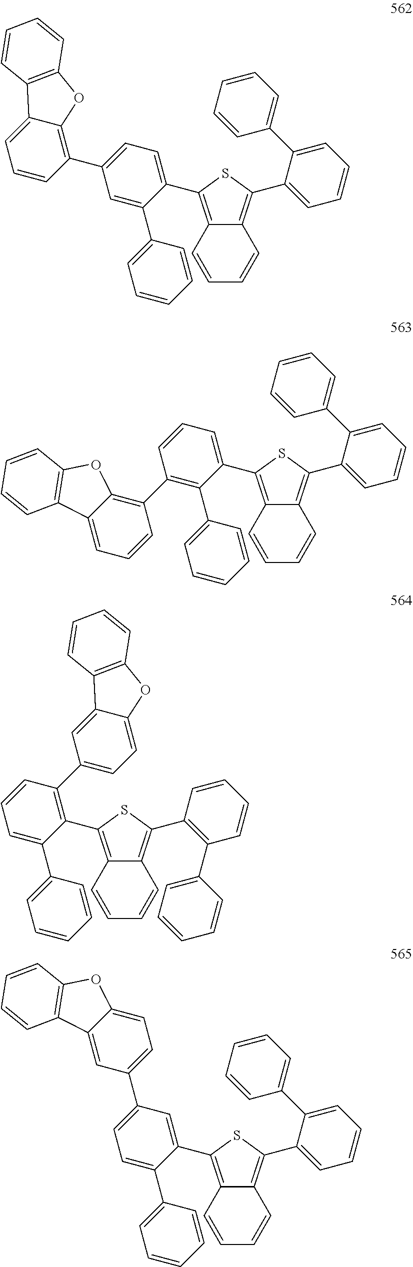

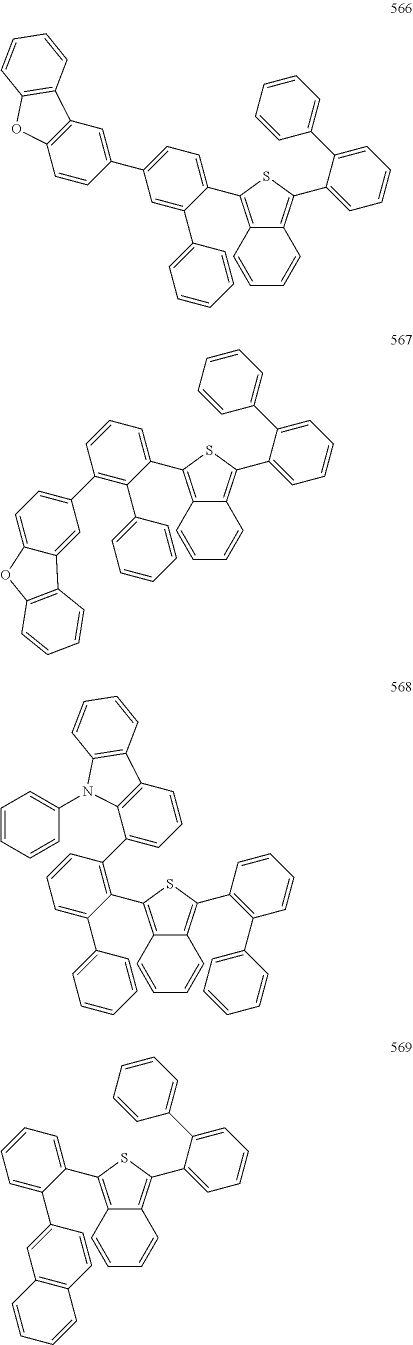 [SMILES: c1ccc(-c2ccccc2-c2sc(-c3ccc(-c4ccc5oc6ccccc6c5c4)cc3-c3ccccc3)c3ccccc23)cc1 ; c1ccc(-c2ccccc2-c2sc(-c3cccc(-c4ccc5oc6ccccc6c5c4)c3-c3ccccc3)c3ccccc23)cc1 ; c1ccc(-c2ccccc2-c2sc(-c3c(-c4ccccc4)cccc3-c3cccc4c5ccccc5n(-c5ccccc5)c34)c3ccccc23)cc1 ; c1ccc(-c2ccccc2-c2sc(-c3ccccc3-c3ccc4ccccc4c3)c3ccccc23)cc1]

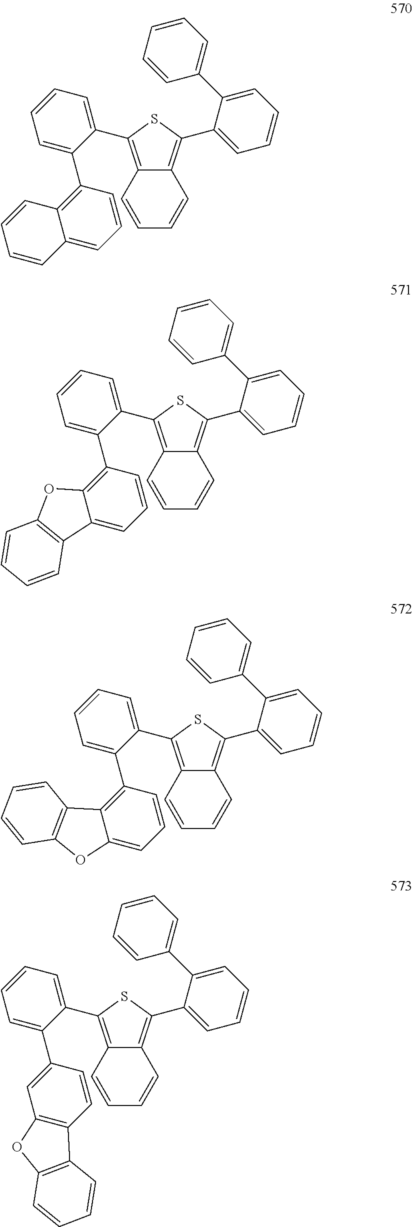 [SMILES: c1ccc(-c2ccccc2-c2sc(-c3ccccc3-c3cccc4ccccc34)c3ccccc23)cc1 ; c1ccc(-c2ccccc2-c2sc(-c3ccccc3-c3cccc4c3oc3ccccc34)c3ccccc23)cc1 ; c1ccc(-c2ccccc2-c2sc(-c3ccccc3-c3cccc4oc5ccccc5c34)c3ccccc23)cc1 ; c1ccc(-c2ccccc2-c2sc(-c3ccccc3-c3ccc4c(c3)oc3ccccc34)c3ccccc23)cc1]

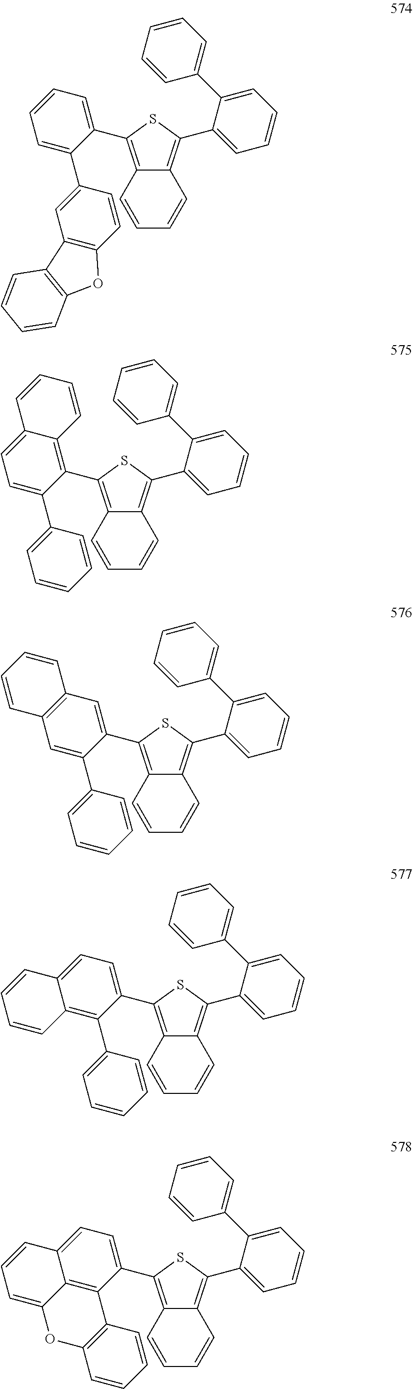

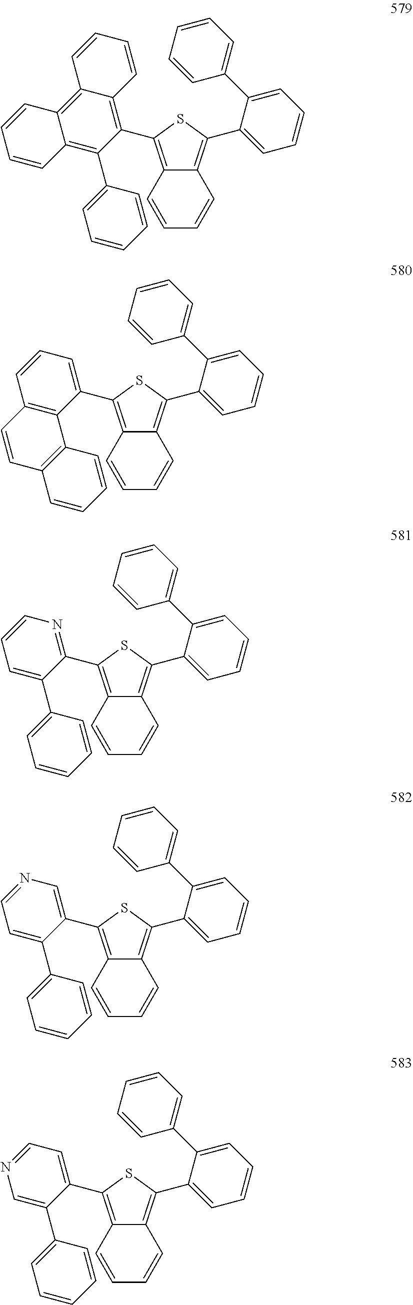

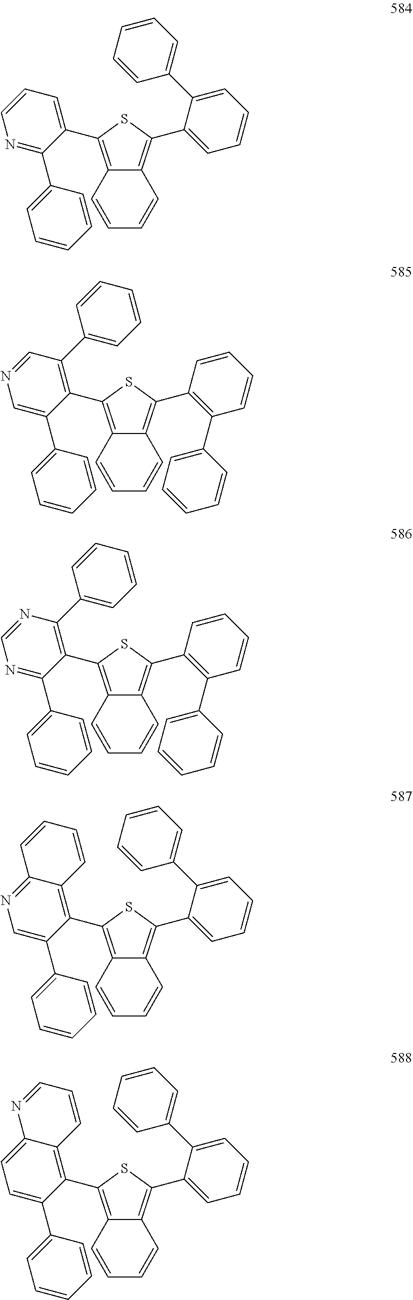

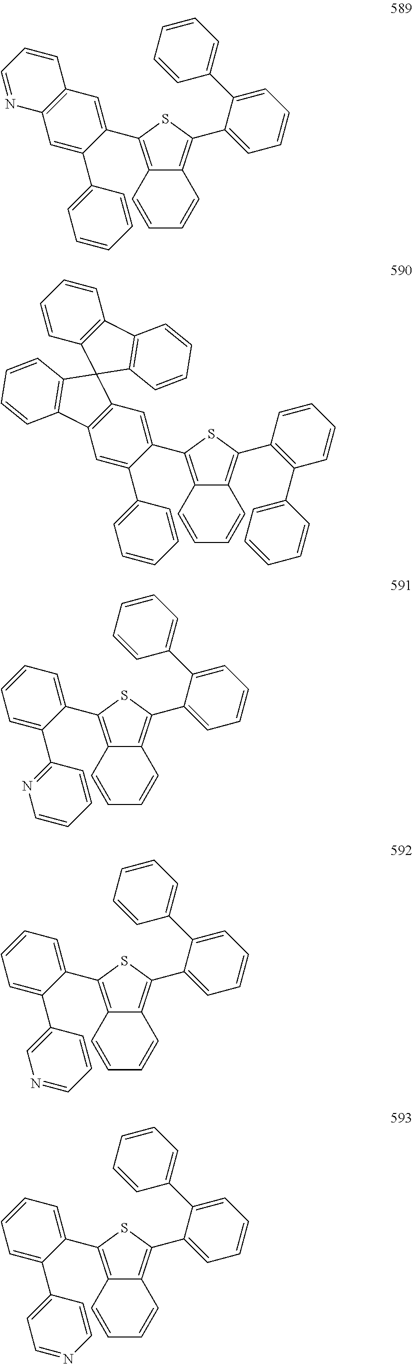

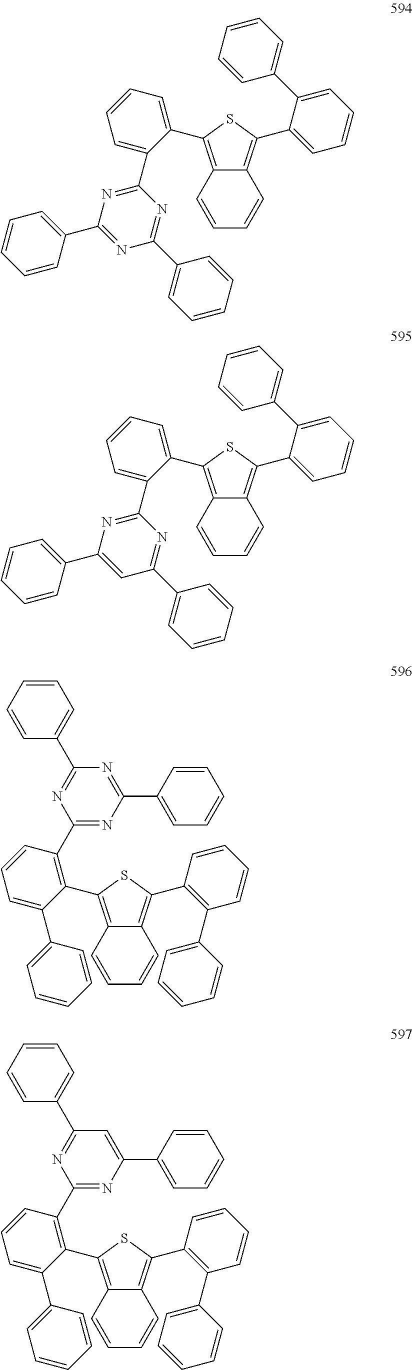

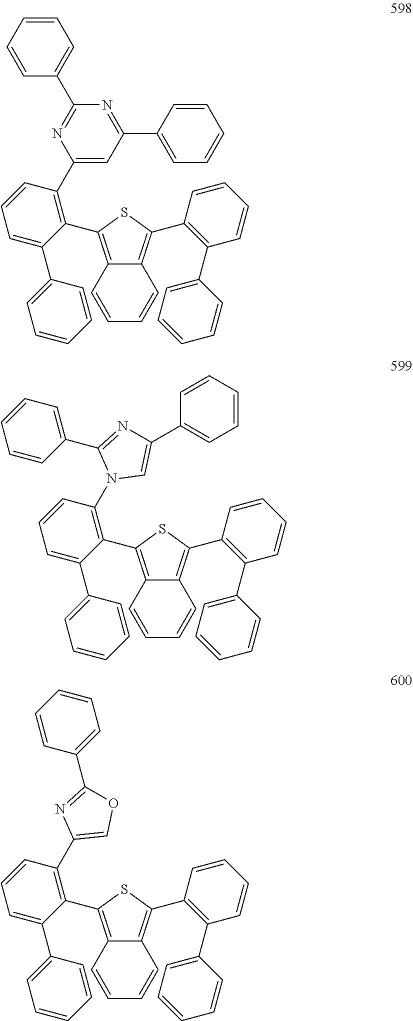

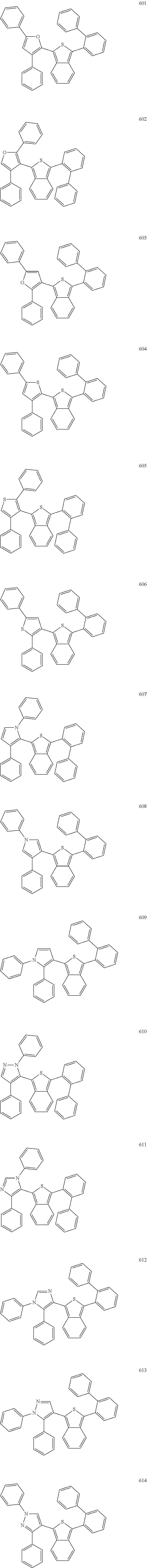

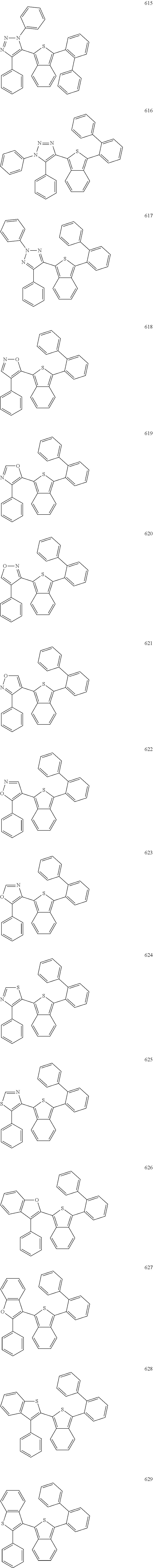

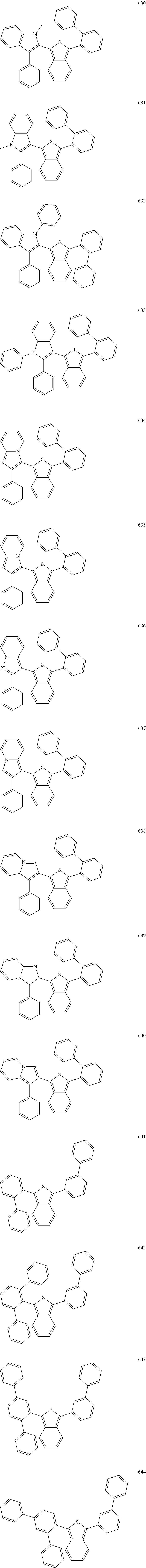

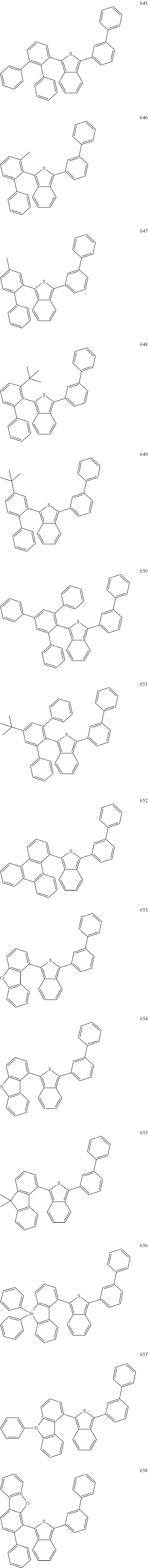

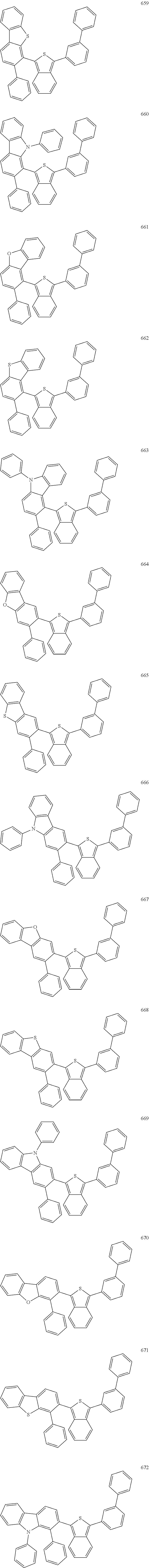

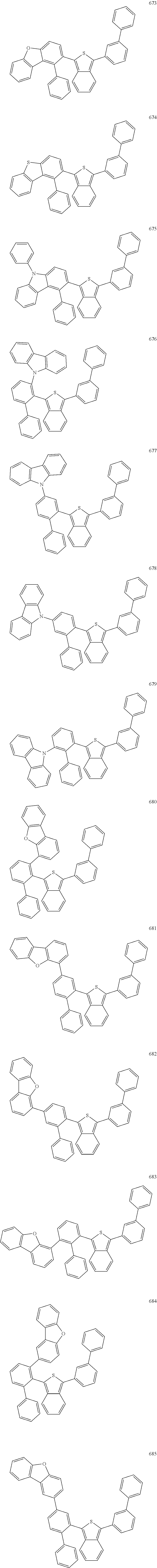

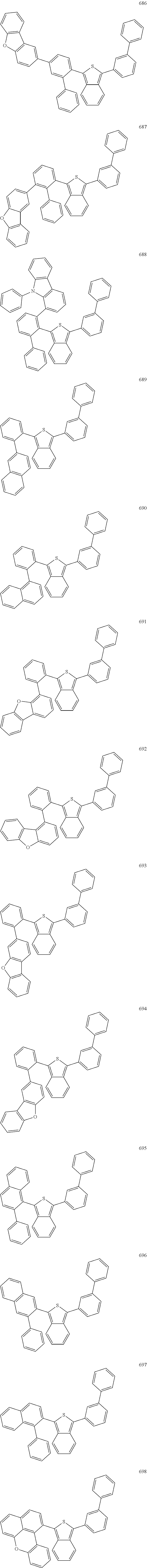

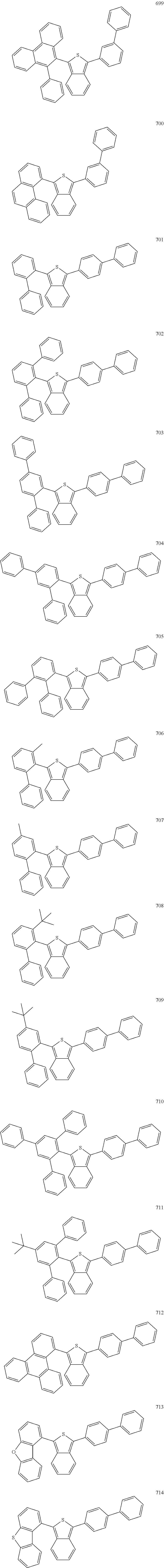

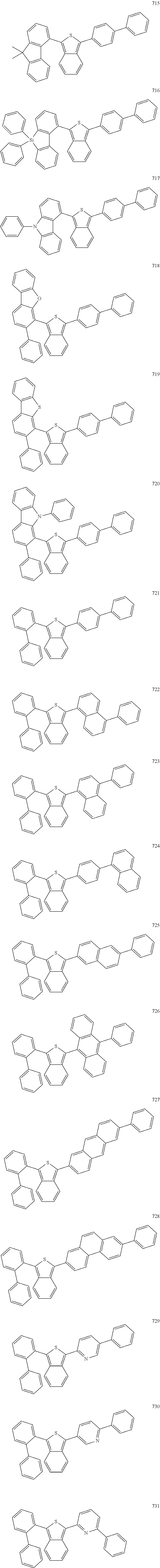

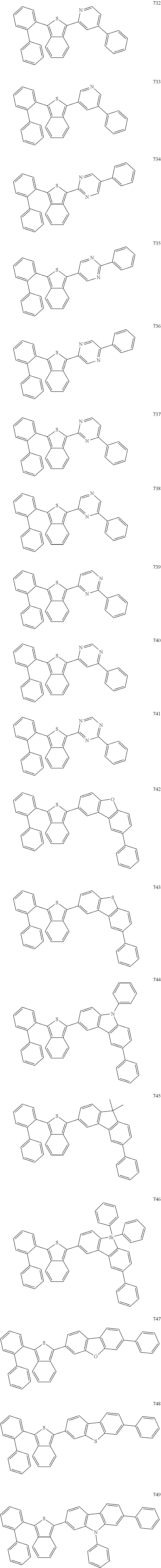

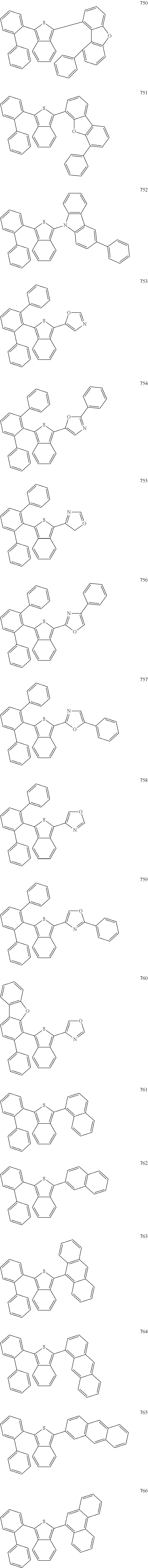

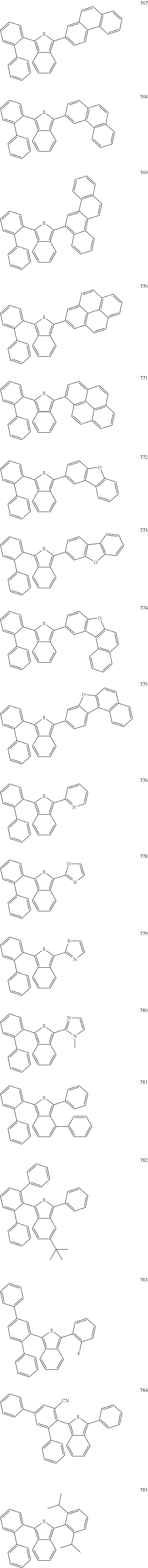

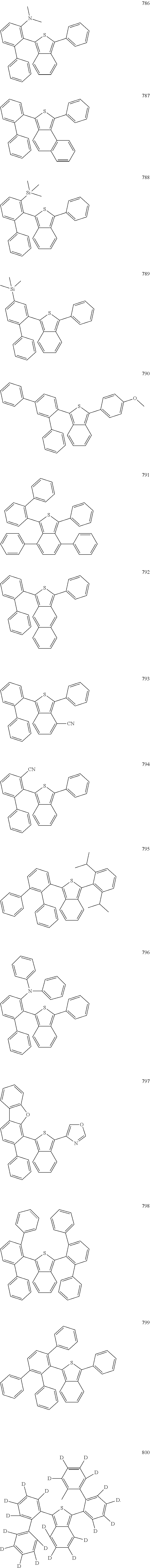

In some embodiments, the fluorescent host may be represented by Formula FH-3:

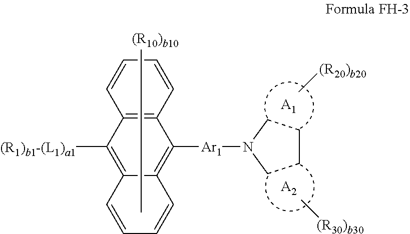

wherein, in Formula FH-3,

Ar₁ may be a group represented by Formula 2:

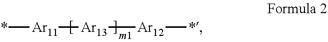

Ar₁ may include at least one cyano group,

A₁ and A₂ may each independently be a C₅-C₃₀ carbocyclic group or a C₁-C₃₀ heterocyclic group,

L₁ may be a substituted or unsubstituted C₅-C₃₀ carbocyclic group or a substituted or unsubstituted C₁-C₃₀ heterocyclic group,

a1 may be 0, 1, 2, or 3,

when a1 is 2 or greater, at least two L₁(s) may be identical to different from each other,

m1 may be 0,1, 2, or 3, and

Ar₁₁ may be a group represented by Formula 4, Ar₁₂ may be a group represented by Formula 5, or Ar₁₃ may be a group represented by Formula 6:

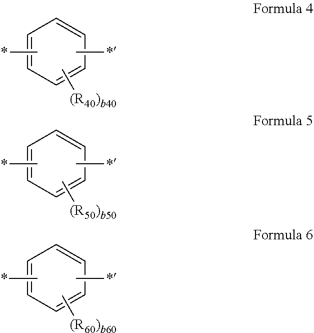

wherein, in Formulae 4 to 6,

R₁, R₁₀, R₂₀, R₃₀, R₄₀, R₅₀, and R₆₀ may each independently be hydrogen, deuterium, —F, —Cl, —Br, —I, —SF₅, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁ cycloalkyl group, a substituted or unsubstituted C₁-C₁₀ heterocycloalkyl group, a substituted or unsubstituted C₃-C₁ cycloalkenyl group, a substituted or unsubstituted C₁-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted C₁-C₆₀ heteroaryloxy group, a substituted or unsubstituted C₁-C₆₀ heteroarylthio group, a substituted or unsubstituted monovalent non-aromatic condensed polycyclic group, a substituted or unsubstituted monovalent non-aromatic condensed heteropolycyclic group, —N(Q₁)(Q₂), —Si(Q₃)(Q₄)(Q₅), —B(Q₆)(Q₇), or —P(═O)(Q₈)(Q₉),

b1 may be an integer from 1 to 5,

when b1 is 2 or greater, at least two R₁(s) may be identical to or different from each other,

b10 may be an integer from 1 to 8,

when b10 is 2 or greater, at least two Rio(s) may be identical to or different from each other,

b20 and b30 may each independently be an integer from 1 to 4,

when b20 is 2 or greater, at least two R₂₀(s) may be identical to or different from each other, and when b30 is 2 or greater, at least two R₃₀ (s) may be identical to or different from each other,

b40, b50, and b60 may each independently be an integer from 1 to 4,

when b40 is 2 or greater, at least two R₄₀ (s) may be identical to or different from each other, when b50 is 2 or greater, at least two R₅₀(s) may be identical to or different from each other, and when b60 is 2 or greater, at least two R₆₀ (s) may be identical to or different from each other, and

* and *′ each indicate a binding site to an adjacent atom.

In some embodiments, the fluorescent host represented by Formula FH-3 may be Group FH3, but embodiments are not limited thereto:

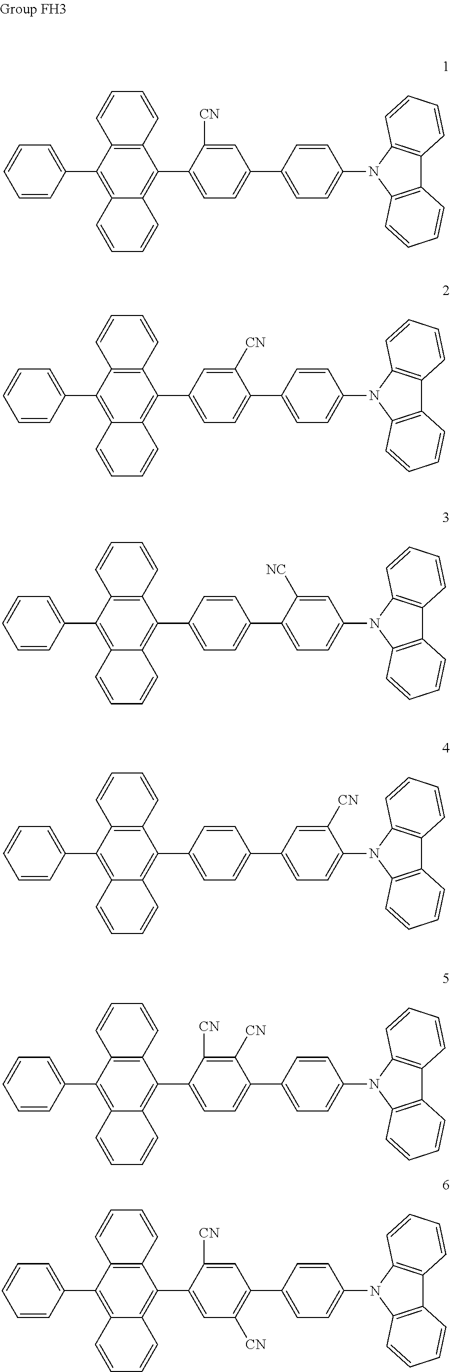 [SMILES: N#Cc1cc(-c2ccc(-n3c4ccccc4c4ccccc43)cc2)ccc1-c1c2ccccc2c(-c2ccccc2)c2ccccc12 ; N#Cc1cc(-c2c3ccccc3c(-c3ccccc3)c3ccccc23)ccc1-c1ccc(-n2c3ccccc3c3ccccc32)cc1 ; N#Cc1cc(-n2c3ccccc3c3ccccc32)ccc1-c1ccc(-c2c3ccccc3c(-c3ccccc3)c3ccccc23)cc1 ; N#Cc1cc(-c2ccc(-c3c4ccccc4c(-c4ccccc4)c4ccccc34)cc2)ccc1-n1c2ccccc2c2ccccc21 ; N#Cc1c(-c2ccc(-n3c4ccccc4c4ccccc43)cc2)ccc(-c2c3ccccc3c(-c3ccccc3)c3ccccc23)c1C#N ; N#Cc1cc(-c2c3ccccc3c(-c3ccccc3)c3ccccc23)c(C#N)cc1-c1ccc(-n2c3ccccc3c3ccccc32)cc1]

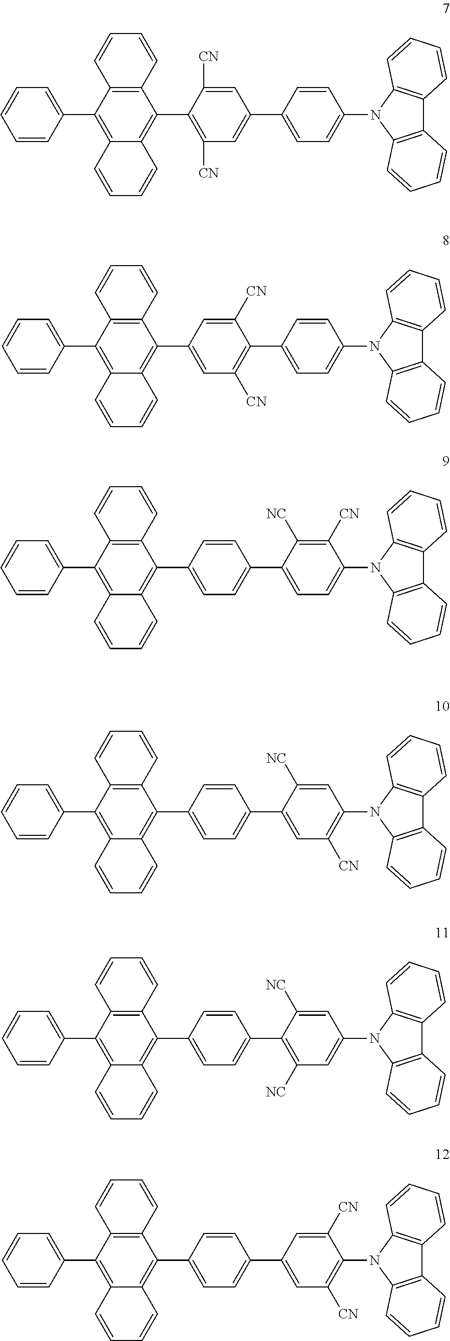

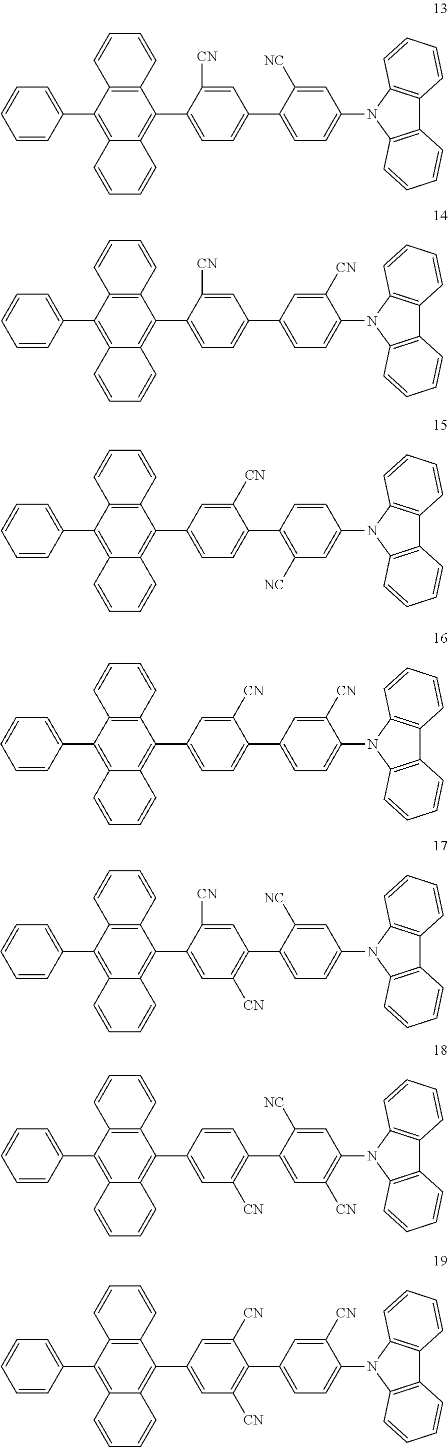

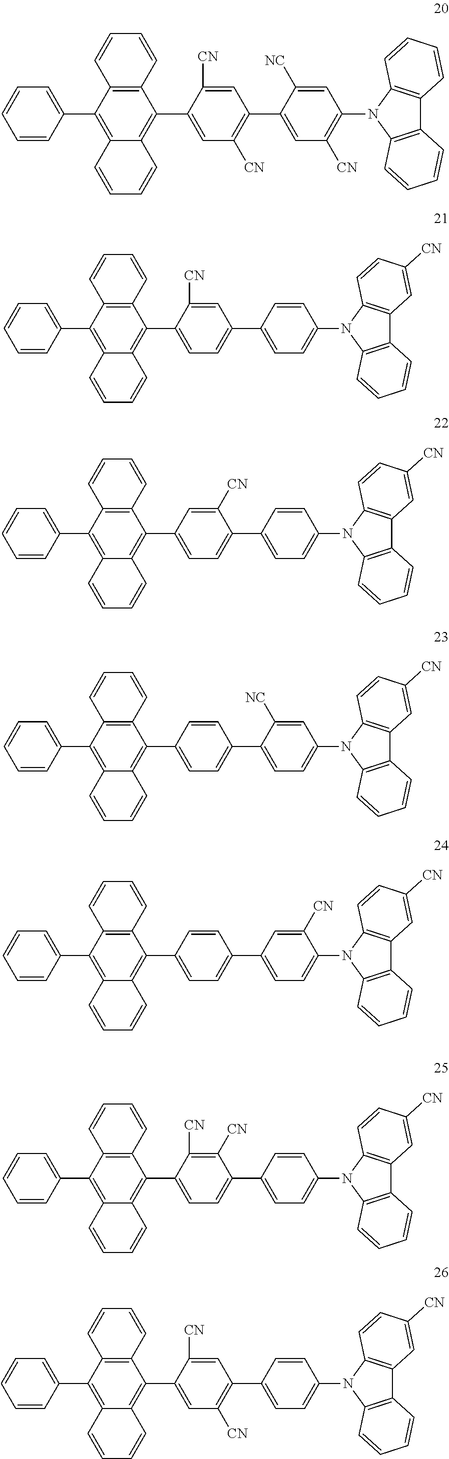

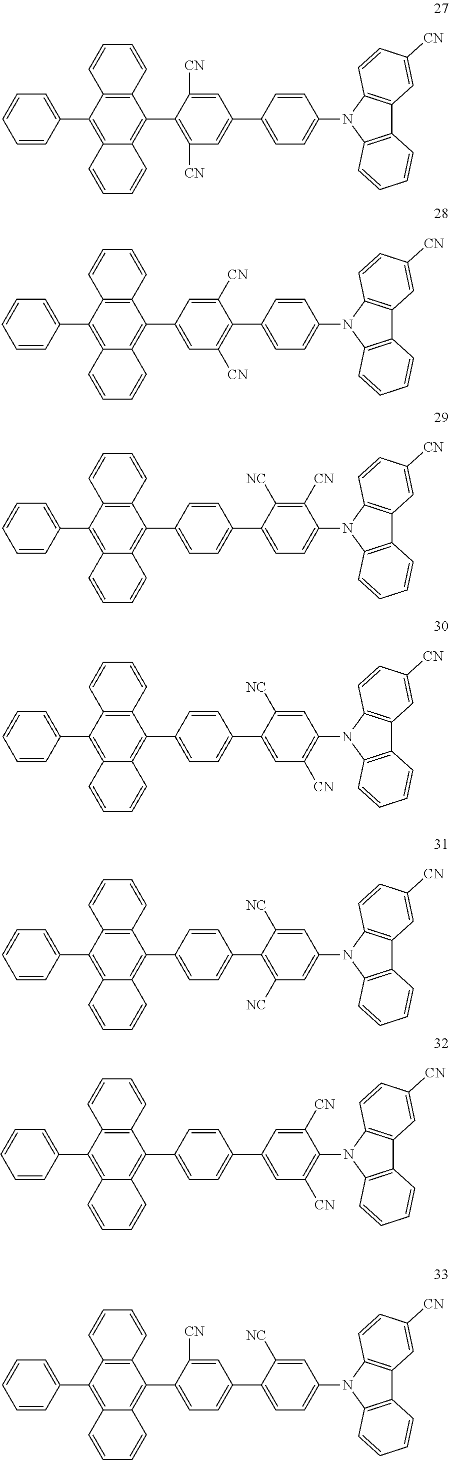

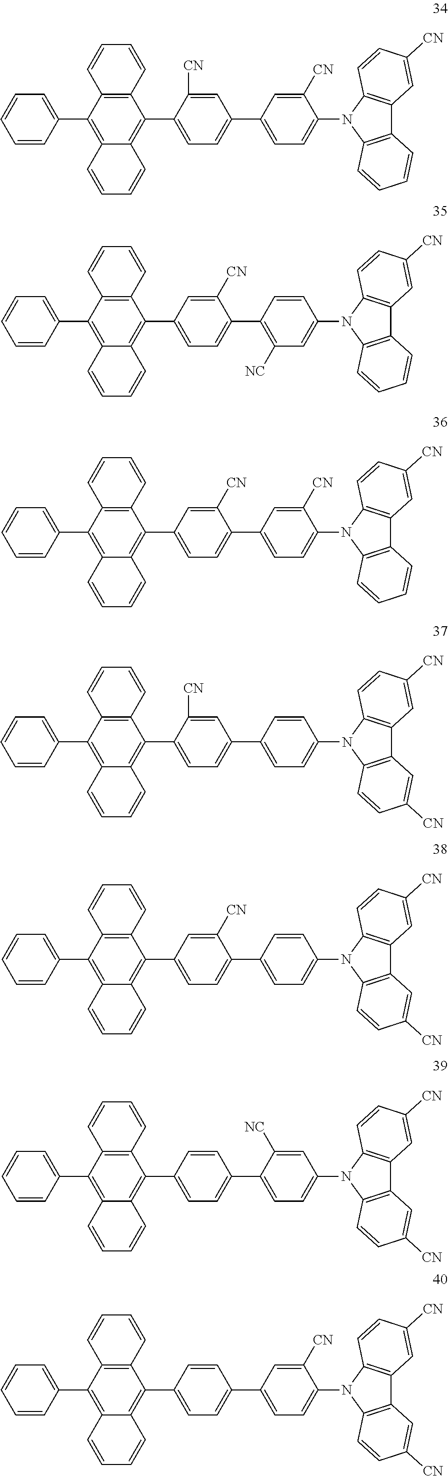

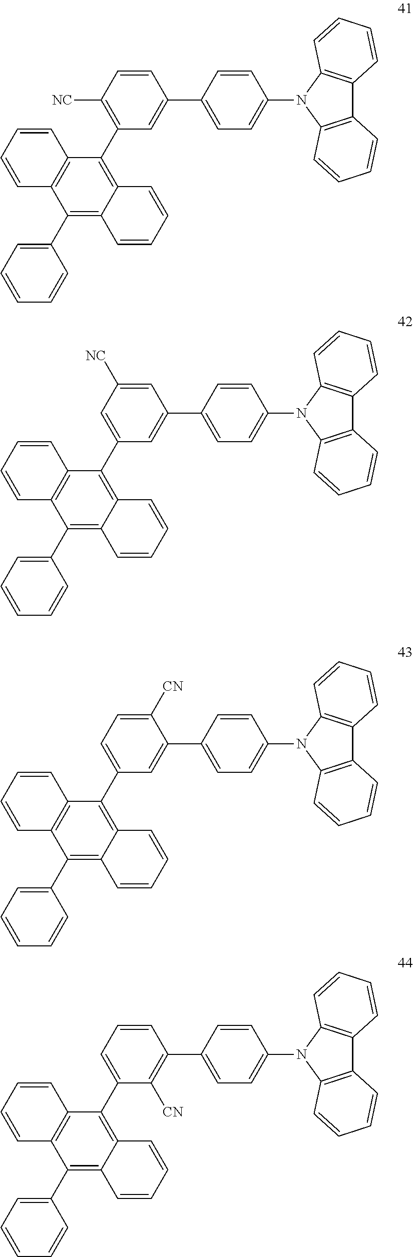 [SMILES: N#Cc1ccc(-c2ccc(-n3c4ccccc4c4ccccc43)cc2)cc1-c1c2ccccc2c(-c2ccccc2)c2ccccc12 ; N#Cc1cc(-c2ccc(-n3c4ccccc4c4ccccc43)cc2)cc(-c2c3ccccc3c(-c3ccccc3)c3ccccc23)c1 ; N#Cc1ccc(-c2c3ccccc3c(-c3ccccc3)c3ccccc23)cc1-c1ccc(-n2c3ccccc3c3ccccc32)cc1 ; N#Cc1c(-c2ccc(-n3c4ccccc4c4ccccc43)cc2)cccc1-c1c2ccccc2c(-c2ccccc2)c2ccccc12]

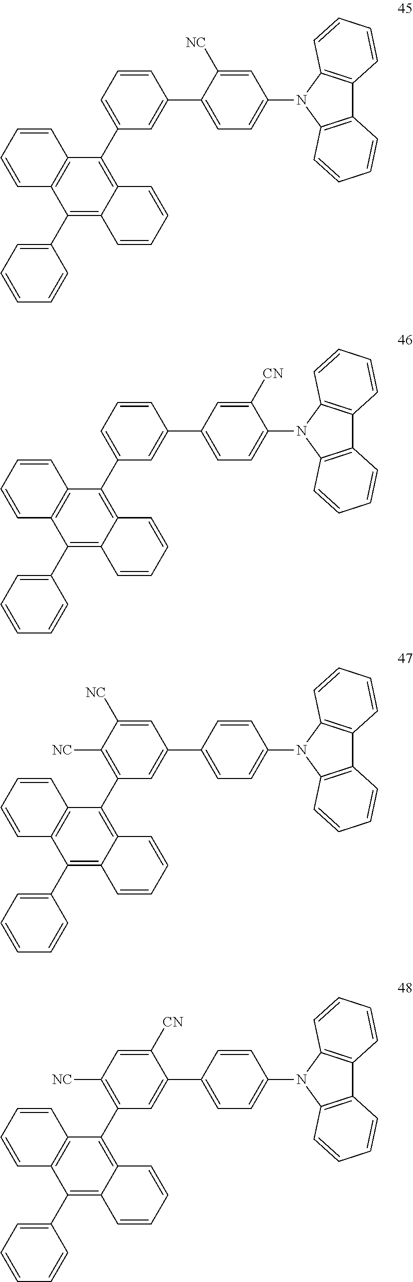 [SMILES: N#Cc1cc(-n2c3ccccc3c3ccccc32)ccc1-c1cccc(-c2c3ccccc3c(-c3ccccc3)c3ccccc23)c1 ; N#Cc1cc(-c2cccc(-c3c4ccccc4c(-c4ccccc4)c4ccccc34)c2)ccc1-n1c2ccccc2c2ccccc21 ; N#Cc1cc(-c2ccc(-n3c4ccccc4c4ccccc43)cc2)cc(-c2c3ccccc3c(-c3ccccc3)c3ccccc23)c1C#N ; N#Cc1cc(C#N)c(-c2c3ccccc3c(-c3ccccc3)c3ccccc23)cc1-c1ccc(-n2c3ccccc3c3ccccc32)cc1]

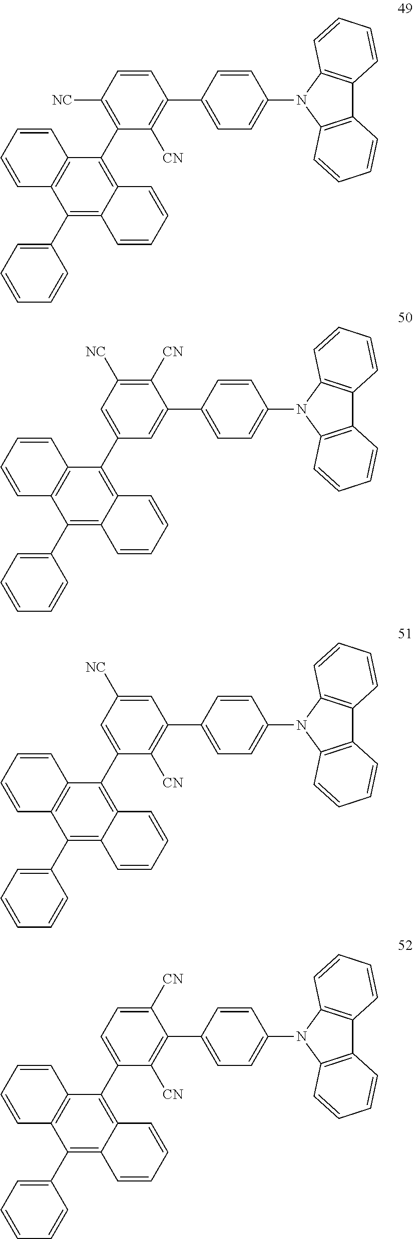 [SMILES: N#Cc1ccc(-c2ccc(-n3c4ccccc4c4ccccc43)cc2)c(C#N)c1-c1c2ccccc2c(-c2ccccc2)c2ccccc12 ; N#Cc1cc(-c2c3ccccc3c(-c3ccccc3)c3ccccc23)cc(-c2ccc(-n3c4ccccc4c4ccccc43)cc2)c1C#N ; N#Cc1cc(-c2ccc(-n3c4ccccc4c4ccccc43)cc2)c(C#N)c(-c2c3ccccc3c(-c3ccccc3)c3ccccc23)c1 ; N#Cc1ccc(-c2c3ccccc3c(-c3ccccc3)c3ccccc23)c(C#N)c1-c1ccc(-n2c3ccccc3c3ccccc32)cc1]

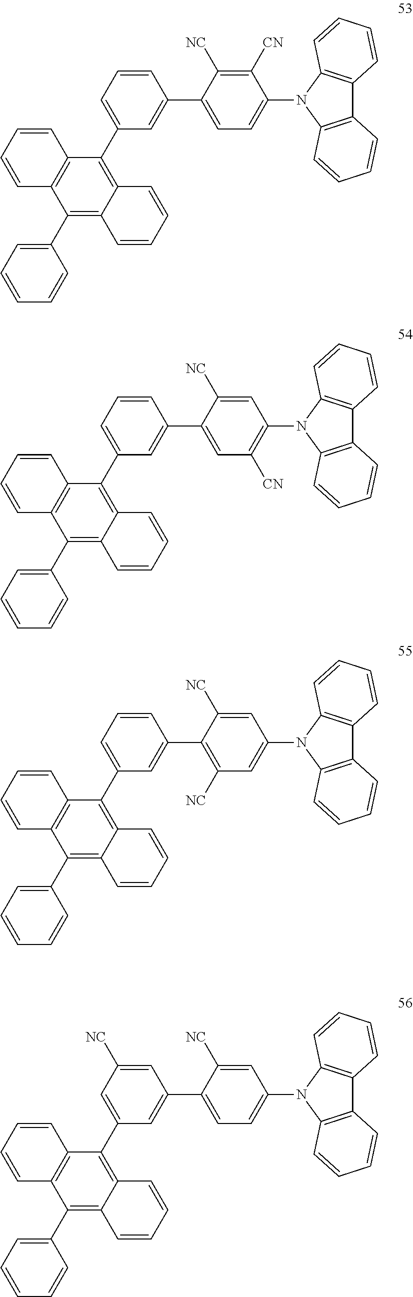 [SMILES: N#Cc1c(-c2cccc(-c3c4ccccc4c(-c4ccccc4)c4ccccc34)c2)ccc(-n2c3ccccc3c3ccccc32)c1C#N ; N#Cc1cc(-n2c3ccccc3c3ccccc32)c(C#N)cc1-c1cccc(-c2c3ccccc3c(-c3ccccc3)c3ccccc23)c1 ; N#Cc1cc(-n2c3ccccc3c3ccccc32)cc(C#N)c1-c1cccc(-c2c3ccccc3c(-c3ccccc3)c3ccccc23)c1 ; N#Cc1cc(-c2ccc(-n3c4ccccc4c4ccccc43)cc2C#N)cc(-c2c3ccccc3c(-c3ccccc3)c3ccccc23)c1]

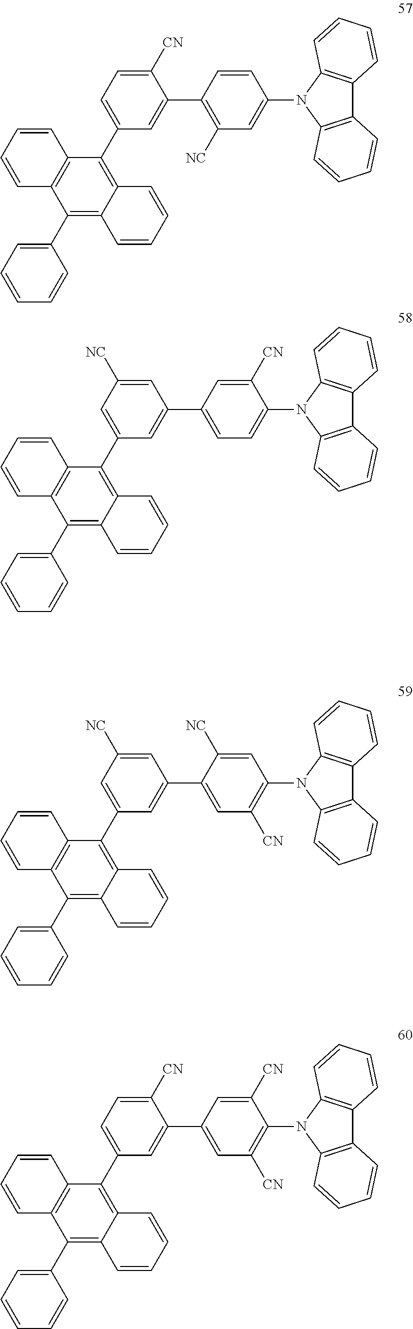 [SMILES: N#Cc1cc(-n2c3ccccc3c3ccccc32)ccc1-c1cc(-c2c3ccccc3c(-c3ccccc3)c3ccccc23)ccc1C#N ; N#Cc1cc(-c2ccc(-n3c4ccccc4c4ccccc43)c(C#N)c2)cc(-c2c3ccccc3c(-c3ccccc3)c3ccccc23)c1 ; N#Cc1cc(-c2cc(C#N)c(-n3c4ccccc4c4ccccc43)cc2C#N)cc(-c2c3ccccc3c(-c3ccccc3)c3ccccc23)c1 ; N#Cc1ccc(-c2c3ccccc3c(-c3ccccc3)c3ccccc23)cc1-c1cc(C#N)c(-n2c3ccccc3c3ccccc32)c(C#N)c1]

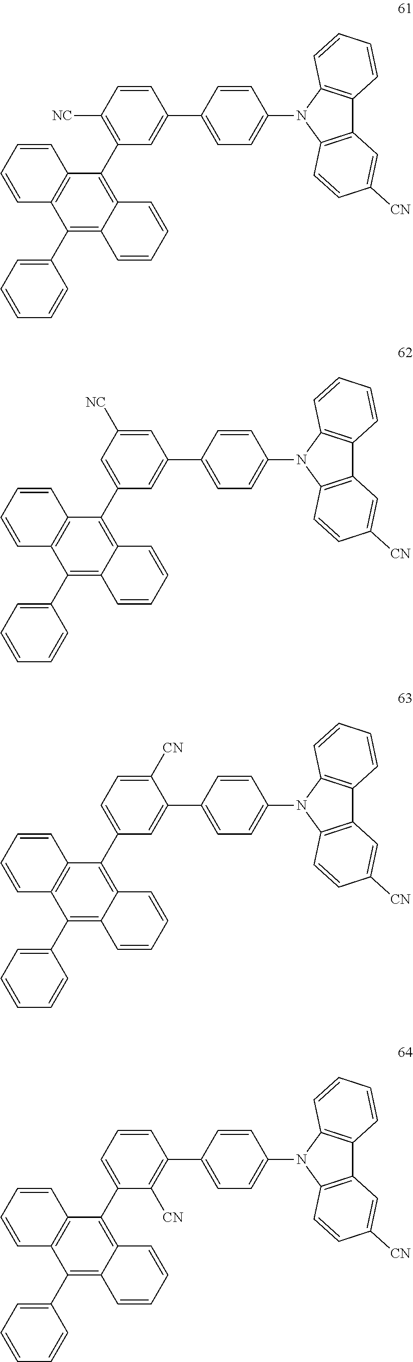 [SMILES: N#Cc1ccc2c(c1)c1ccccc1n2-c1ccc(-c2ccc(C#N)c(-c3c4ccccc4c(-c4ccccc4)c4ccccc34)c2)cc1 ; N#Cc1cc(-c2ccc(-n3c4ccccc4c4cc(C#N)ccc43)cc2)cc(-c2c3ccccc3c(-c3ccccc3)c3ccccc23)c1 ; N#Cc1ccc2c(c1)c1ccccc1n2-c1ccc(-c2cc(-c3c4ccccc4c(-c4ccccc4)c4ccccc34)ccc2C#N)cc1 ; N#Cc1ccc2c(c1)c1ccccc1n2-c1ccc(-c2cccc(-c3c4ccccc4c(-c4ccccc4)c4ccccc34)c2C#N)cc1]

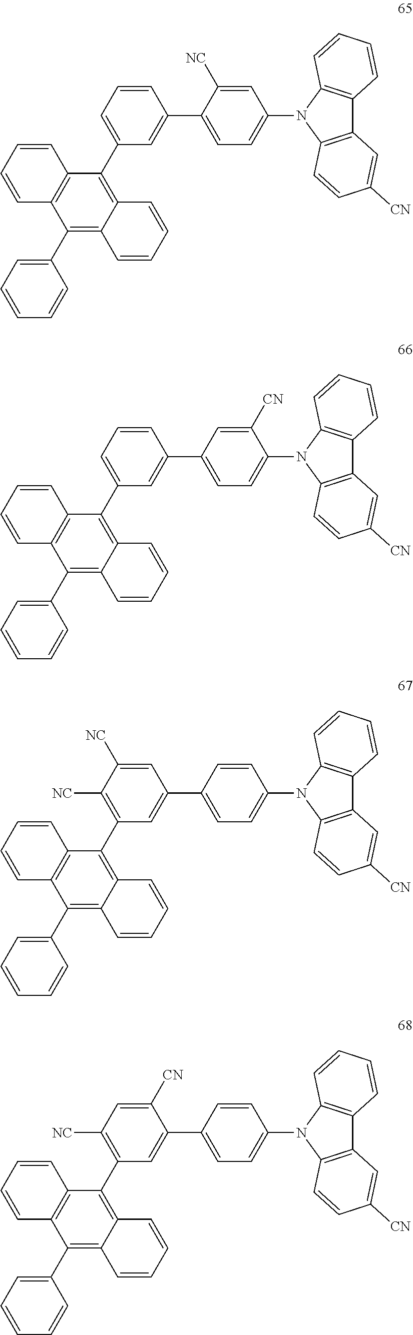 [SMILES: N#Cc1ccc2c(c1)c1ccccc1n2-c1ccc(-c2cccc(-c3c4ccccc4c(-c4ccccc4)c4ccccc34)c2)c(C#N)c1 ; N#Cc1ccc2c(c1)c1ccccc1n2-c1ccc(-c2cccc(-c3c4ccccc4c(-c4ccccc4)c4ccccc34)c2)cc1C#N ; N#Cc1ccc2c(c1)c1ccccc1n2-c1ccc(-c2cc(C#N)c(C#N)c(-c3c4ccccc4c(-c4ccccc4)c4ccccc34)c2)cc1 ; N#Cc1ccc2c(c1)c1ccccc1n2-c1ccc(-c2cc(-c3c4ccccc4c(-c4ccccc4)c4ccccc34)c(C#N)cc2C#N)cc1]

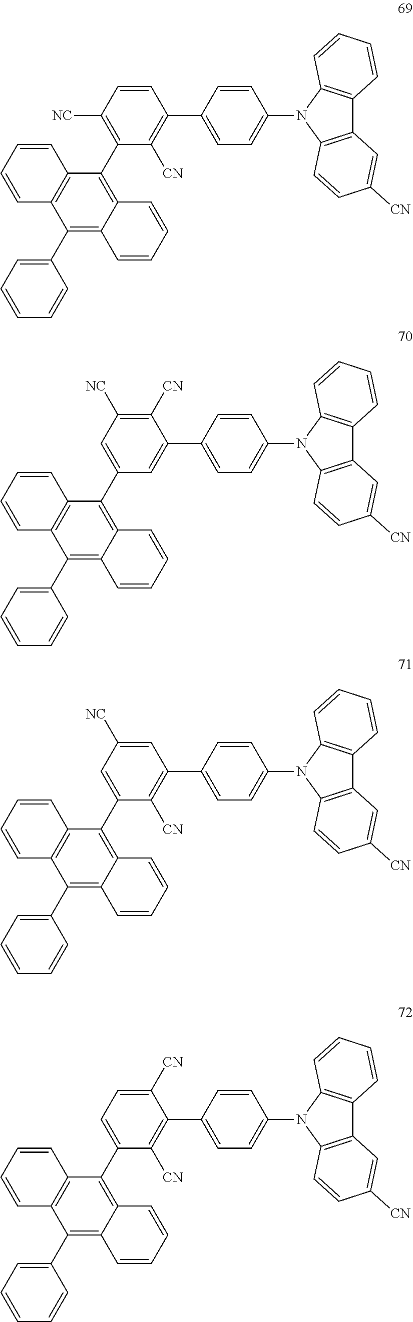 [SMILES: N#Cc1ccc2c(c1)c1ccccc1n2-c1ccc(-c2ccc(C#N)c(-c3c4ccccc4c(-c4ccccc4)c4ccccc34)c2C#N)cc1 ; N#Cc1ccc2c(c1)c1ccccc1n2-c1ccc(-c2cc(-c3c4ccccc4c(-c4ccccc4)c4ccccc34)cc(C#N)c2C#N)cc1 ; N#Cc1cc(-c2ccc(-n3c4ccccc4c4cc(C#N)ccc43)cc2)c(C#N)c(-c2c3ccccc3c(-c3ccccc3)c3ccccc23)c1 ; N#Cc1ccc2c(c1)c1ccccc1n2-c1ccc(-c2c(C#N)ccc(-c3c4ccccc4c(-c4ccccc4)c4ccccc34)c2C#N)cc1]

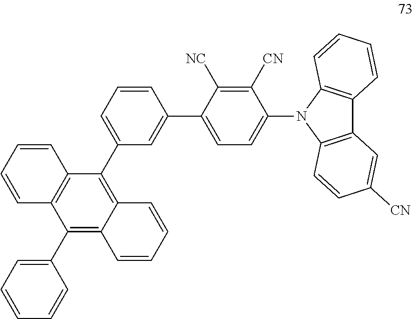 [SMILES: N#Cc1ccc2c(c1)c1ccccc1n2-c1ccc(-c2cccc(-c3c4ccccc4c(-c4ccccc4)c4ccccc34)c2)c(C#N)c1C#N]

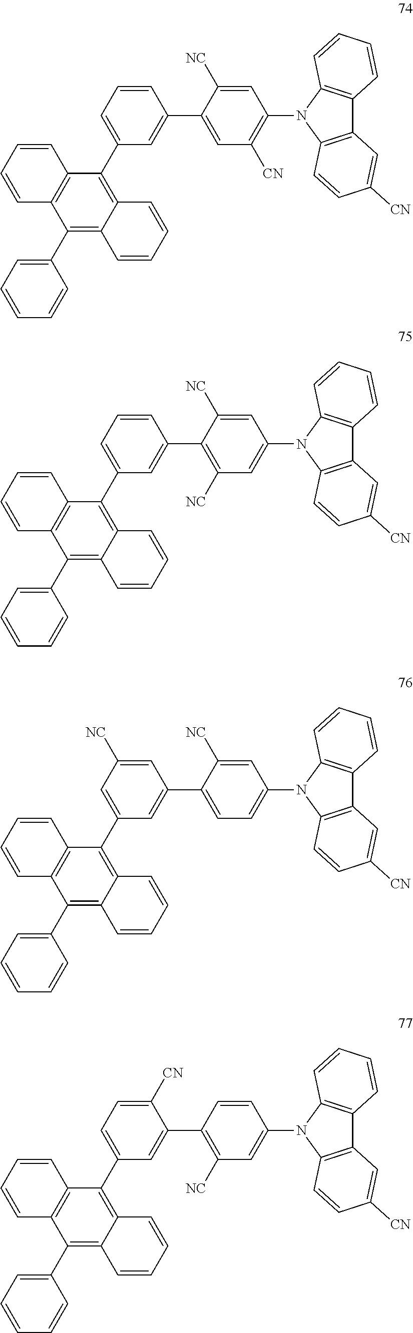 [SMILES: N#Cc1ccc2c(c1)c1ccccc1n2-c1cc(C#N)c(-c2cccc(-c3c4ccccc4c(-c4ccccc4)c4ccccc34)c2)cc1C#N ; N#Cc1ccc2c(c1)c1ccccc1n2-c1cc(C#N)c(-c2cccc(-c3c4ccccc4c(-c4ccccc4)c4ccccc34)c2)c(C#N)c1 ; N#Cc1cc(-c2ccc(-n3c4ccccc4c4cc(C#N)ccc43)cc2C#N)cc(-c2c3ccccc3c(-c3ccccc3)c3ccccc23)c1 ; N#Cc1ccc2c(c1)c1ccccc1n2-c1ccc(-c2cc(-c3c4ccccc4c(-c4ccccc4)c4ccccc34)ccc2C#N)c(C#N)c1]

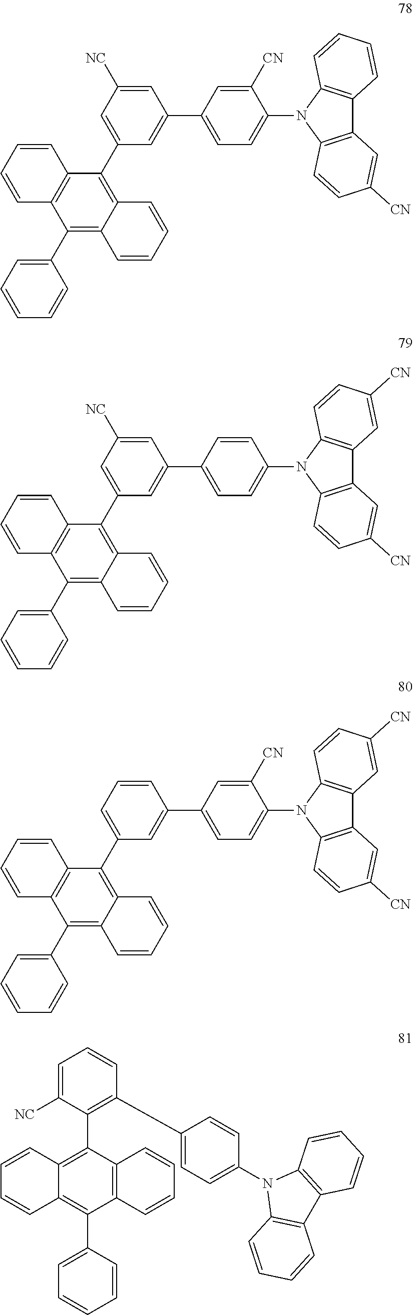 [SMILES: N#Cc1cc(-c2ccc(-n3c4ccccc4c4cc(C#N)ccc43)c(C#N)c2)cc(-c2c3ccccc3c(-c3ccccc3)c3ccccc23)c1 ; N#Cc1cc(-c2ccc(-n3c4ccc(C#N)cc4c4cc(C#N)ccc43)cc2)cc(-c2c3ccccc3c(-c3ccccc3)c3ccccc23)c1 ; N#Cc1ccc2c(c1)c1cc(C#N)ccc1n2-c1ccc(-c2cccc(-c3c4ccccc4c(-c4ccccc4)c4ccccc34)c2)cc1C#N ; N#Cc1cccc(-c2ccc(-n3c4ccccc4c4ccccc43)cc2)c1-c1c2ccccc2c(-c2ccccc2)c2ccccc12]

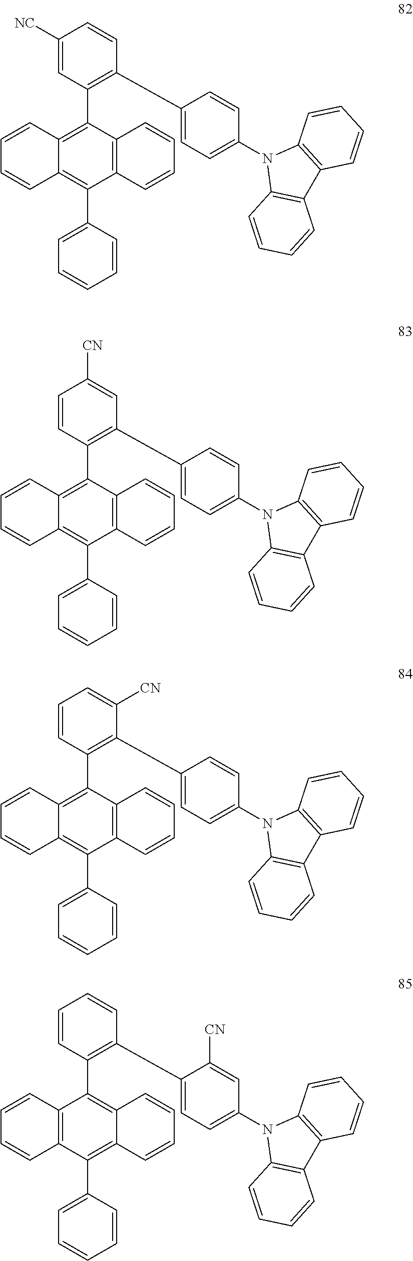

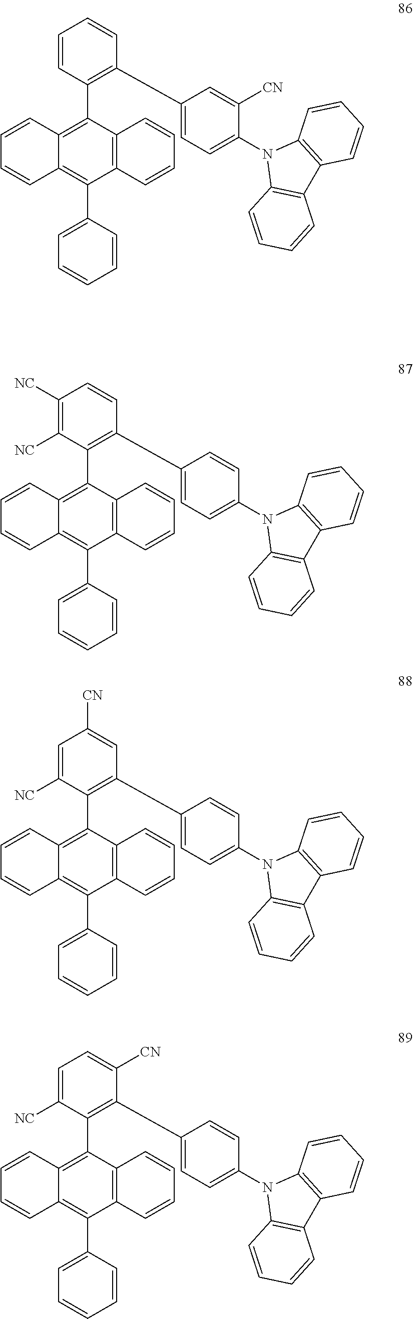

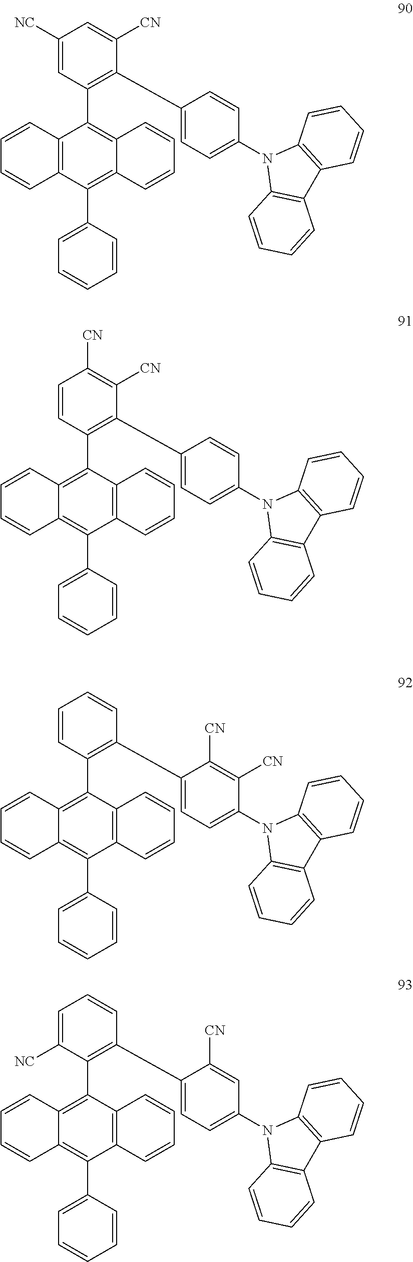

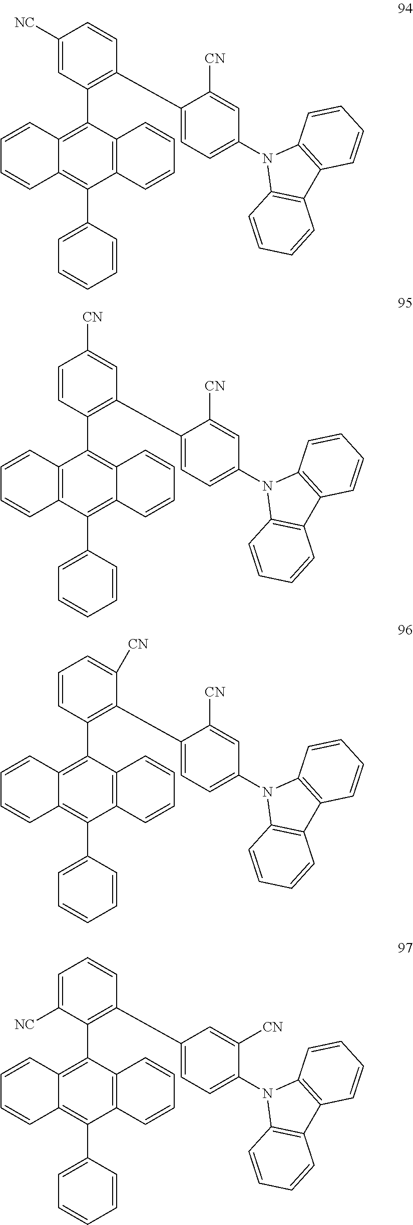

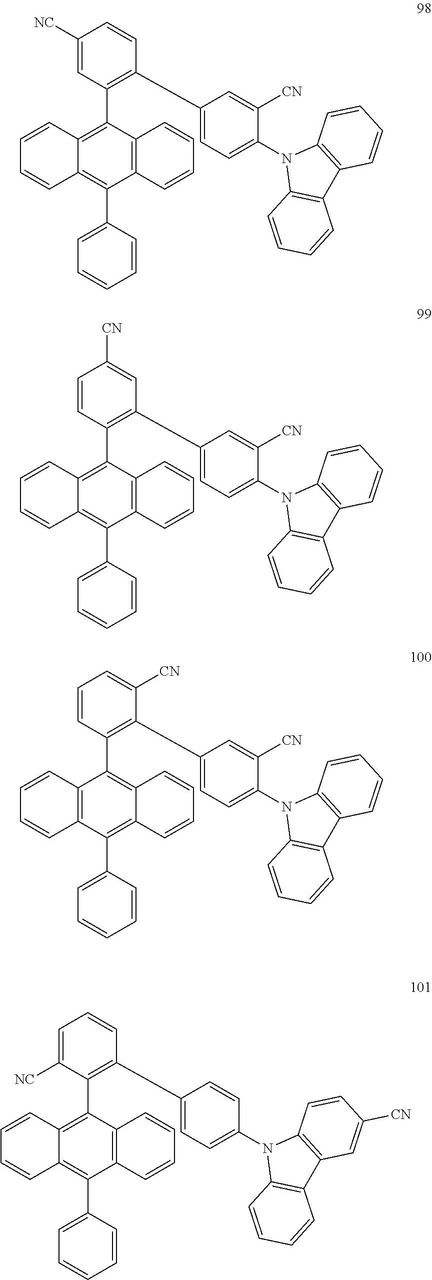

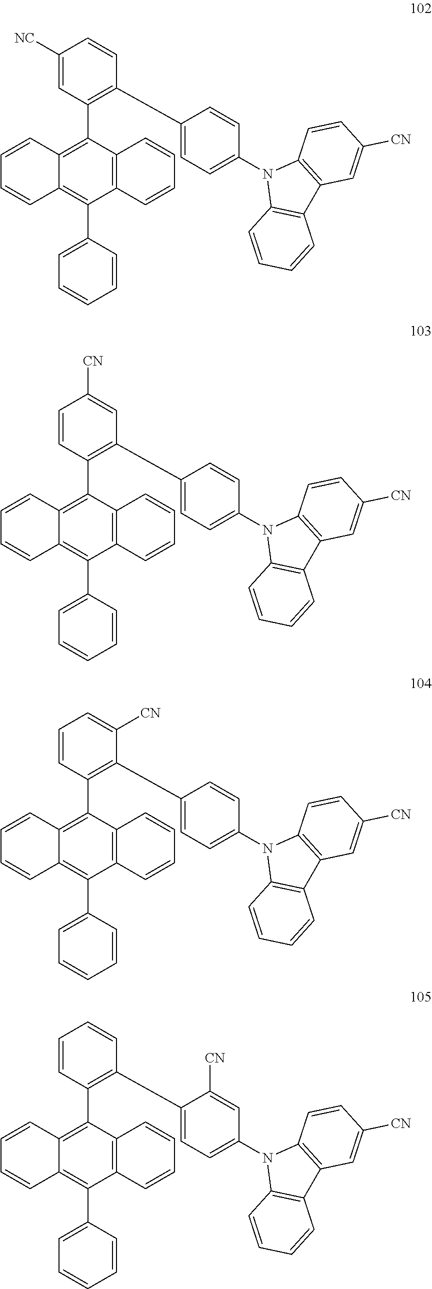

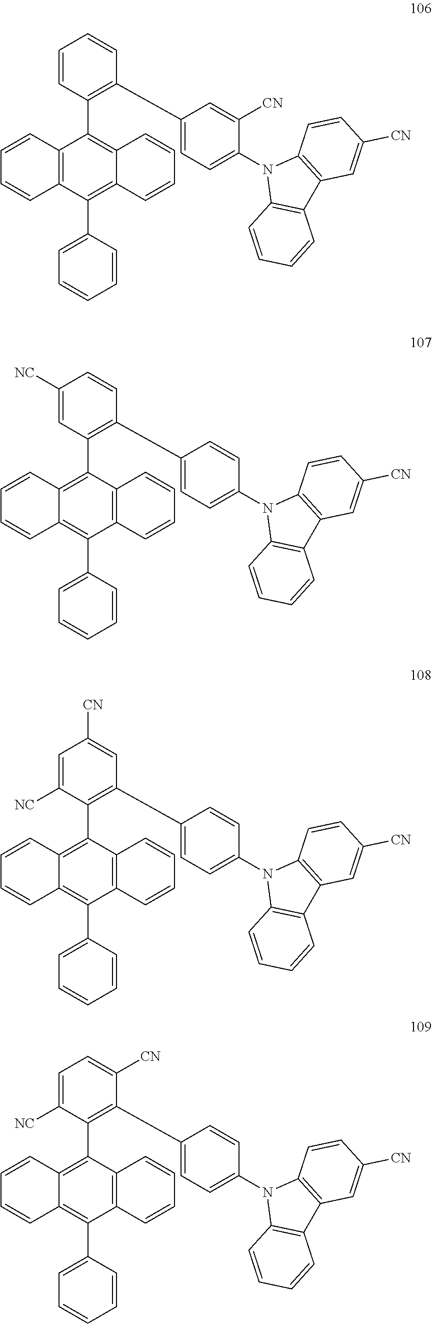 [SMILES: N#Cc1ccc2c(c1)c1ccccc1n2-c1ccc(-c2ccccc2-c2c3ccccc3c(-c3ccccc3)c3ccccc23)cc1C#N ; N#Cc1ccc(-c2ccc(-n3c4ccccc4c4cc(C#N)ccc43)cc2)c(-c2c3ccccc3c(-c3ccccc3)c3ccccc23)c1 ; N#Cc1cc(C#N)c(-c2c3ccccc3c(-c3ccccc3)c3ccccc23)c(-c2ccc(-n3c4ccccc4c4cc(C#N)ccc43)cc2)c1 ; N#Cc1ccc2c(c1)c1ccccc1n2-c1ccc(-c2c(C#N)ccc(C#N)c2-c2c3ccccc3c(-c3ccccc3)c3ccccc23)cc1]

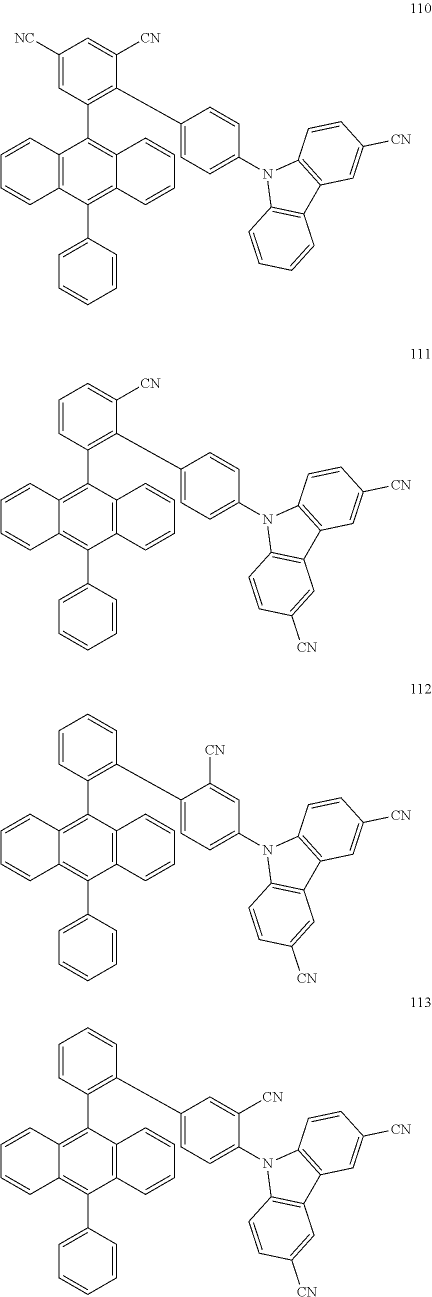 [SMILES: N#Cc1cc(C#N)c(-c2ccc(-n3c4ccccc4c4cc(C#N)ccc43)cc2)c(-c2c3ccccc3c(-c3ccccc3)c3ccccc23)c1 ; N#Cc1ccc2c(c1)c1cc(C#N)ccc1n2-c1ccc(-c2c(C#N)cccc2-c2c3ccccc3c(-c3ccccc3)c3ccccc23)cc1 ; N#Cc1ccc2c(c1)c1cc(C#N)ccc1n2-c1ccc(-c2ccccc2-c2c3ccccc3c(-c3ccccc3)c3ccccc23)c(C#N)c1 ; N#Cc1ccc2c(c1)c1cc(C#N)ccc1n2-c1ccc(-c2ccccc2-c2c3ccccc3c(-c3ccccc3)c3ccccc23)cc1C#N]

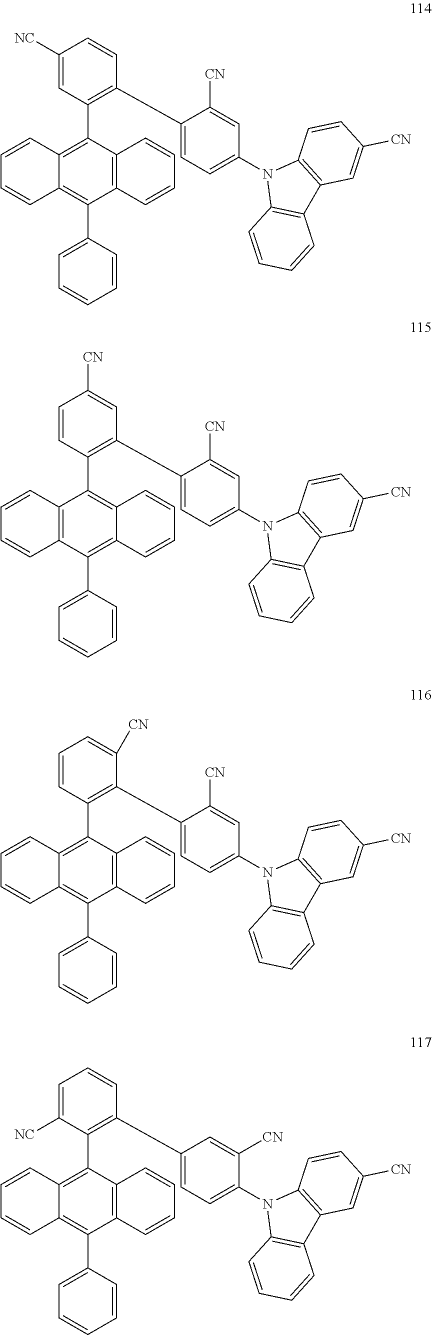 [SMILES: N#Cc1ccc(-c2ccc(-n3c4ccccc4c4cc(C#N)ccc43)cc2C#N)c(-c2c3ccccc3c(-c3ccccc3)c3ccccc23)c1 ; N#Cc1ccc(-c2c3ccccc3c(-c3ccccc3)c3ccccc23)c(-c2ccc(-n3c4ccccc4c4cc(C#N)ccc43)cc2C#N)c1 ; N#Cc1ccc2c(c1)c1ccccc1n2-c1ccc(-c2c(C#N)cccc2-c2c3ccccc3c(-c3ccccc3)c3ccccc23)c(C#N)c1 ; N#Cc1ccc2c(c1)c1ccccc1n2-c1ccc(-c2cccc(C#N)c2-c2c3ccccc3c(-c3ccccc3)c3ccccc23)cc1C#N]

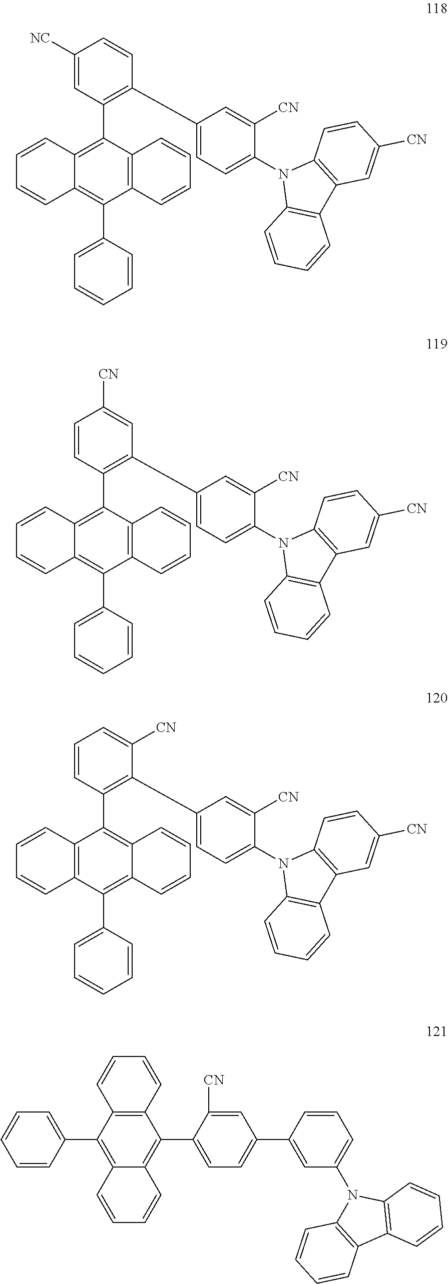 [SMILES: N#Cc1ccc(-c2ccc(-n3c4ccccc4c4cc(C#N)ccc43)c(C#N)c2)c(-c2c3ccccc3c(-c3ccccc3)c3ccccc23)c1 ; N#Cc1ccc(-c2c3ccccc3c(-c3ccccc3)c3ccccc23)c(-c2ccc(-n3c4ccccc4c4cc(C#N)ccc43)c(C#N)c2)c1 ; N#Cc1ccc2c(c1)c1ccccc1n2-c1ccc(-c2c(C#N)cccc2-c2c3ccccc3c(-c3ccccc3)c3ccccc23)cc1C#N ; N#Cc1cc(-c2cccc(-n3c4ccccc4c4ccccc43)c2)ccc1-c1c2ccccc2c(-c2ccccc2)c2ccccc12]

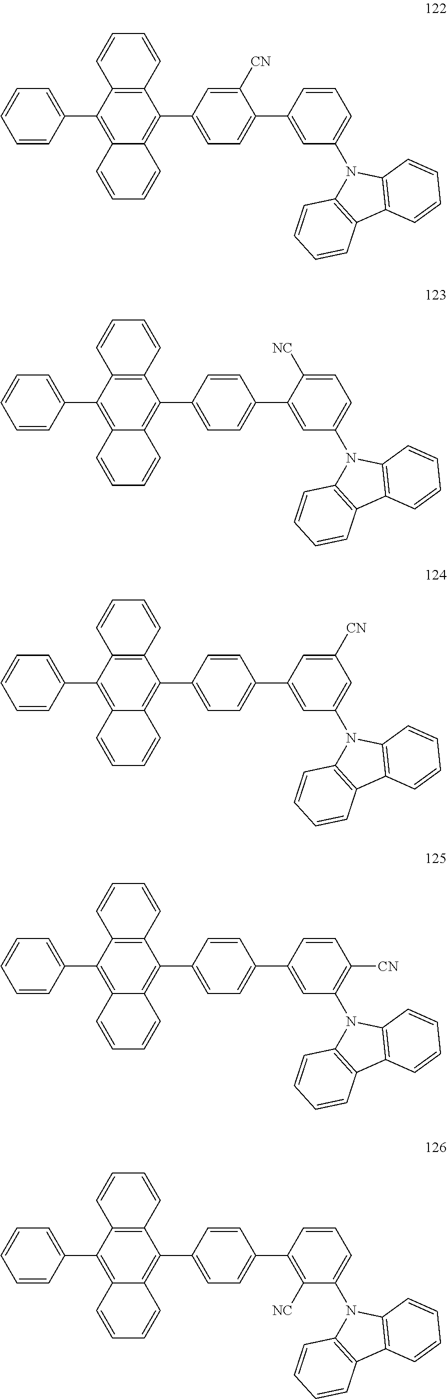 [SMILES: N#Cc1cc(-c2c3ccccc3c(-c3ccccc3)c3ccccc23)ccc1-c1cccc(-n2c3ccccc3c3ccccc32)c1 ; N#Cc1ccc(-n2c3ccccc3c3ccccc32)cc1-c1ccc(-c2c3ccccc3c(-c3ccccc3)c3ccccc23)cc1 ; N#Cc1cc(-c2ccc(-c3c4ccccc4c(-c4ccccc4)c4ccccc34)cc2)cc(-n2c3ccccc3c3ccccc32)c1 ; N#Cc1ccc(-c2ccc(-c3c4ccccc4c(-c4ccccc4)c4ccccc34)cc2)cc1-n1c2ccccc2c2ccccc21 ; N#Cc1c(-c2ccc(-c3c4ccccc4c(-c4ccccc4)c4ccccc34)cc2)cccc1-n1c2ccccc2c2ccccc21]

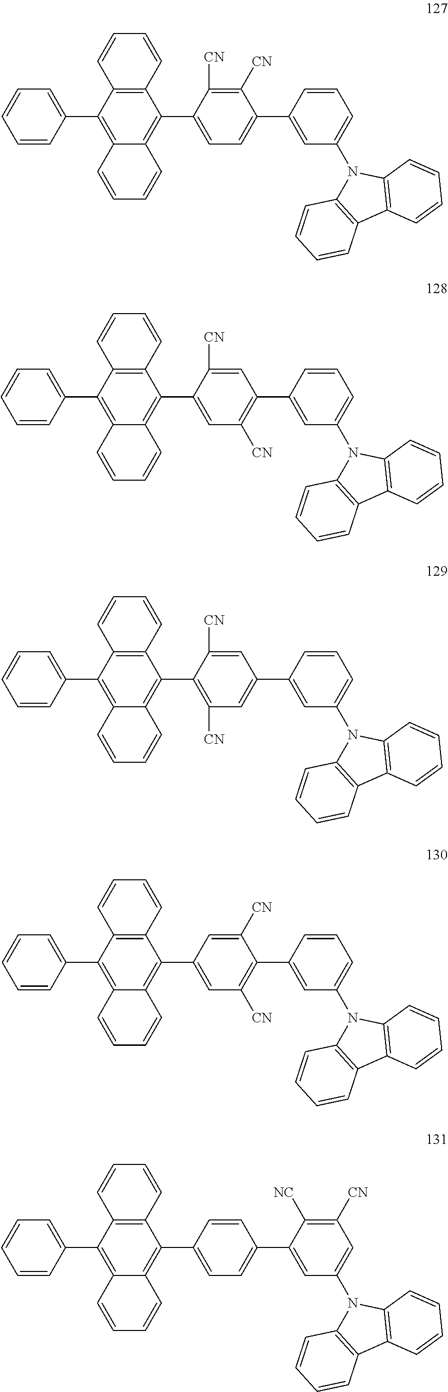 [SMILES: N#Cc1c(-c2cccc(-n3c4ccccc4c4ccccc43)c2)ccc(-c2c3ccccc3c(-c3ccccc3)c3ccccc23)c1C#N ; N#Cc1cc(-c2c3ccccc3c(-c3ccccc3)c3ccccc23)c(C#N)cc1-c1cccc(-n2c3ccccc3c3ccccc32)c1 ; N#Cc1cc(-c2cccc(-n3c4ccccc4c4ccccc43)c2)cc(C#N)c1-c1c2ccccc2c(-c2ccccc2)c2ccccc12 ; N#Cc1cc(-c2c3ccccc3c(-c3ccccc3)c3ccccc23)cc(C#N)c1-c1cccc(-n2c3ccccc3c3ccccc32)c1 ; N#Cc1cc(-n2c3ccccc3c3ccccc32)cc(-c2ccc(-c3c4ccccc4c(-c4ccccc4)c4ccccc34)cc2)c1C#N]

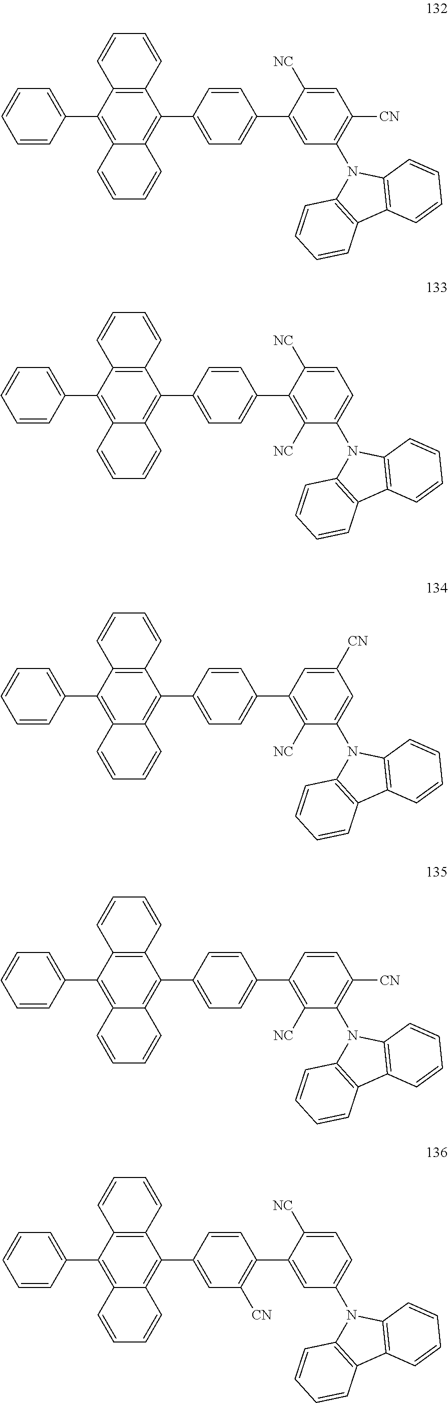 [SMILES: N#Cc1cc(C#N)c(-n2c3ccccc3c3ccccc32)cc1-c1ccc(-c2c3ccccc3c(-c3ccccc3)c3ccccc23)cc1 ; N#Cc1ccc(-n2c3ccccc3c3ccccc32)c(C#N)c1-c1ccc(-c2c3ccccc3c(-c3ccccc3)c3ccccc23)cc1 ; N#Cc1cc(-c2ccc(-c3c4ccccc4c(-c4ccccc4)c4ccccc34)cc2)c(C#N)c(-n2c3ccccc3c3ccccc32)c1 ; N#Cc1ccc(-c2ccc(-c3c4ccccc4c(-c4ccccc4)c4ccccc34)cc2)c(C#N)c1-n1c2ccccc2c2ccccc21 ; N#Cc1cc(-c2c3ccccc3c(-c3ccccc3)c3ccccc23)ccc1-c1cc(-n2c3ccccc3c3ccccc32)ccc1C#N]

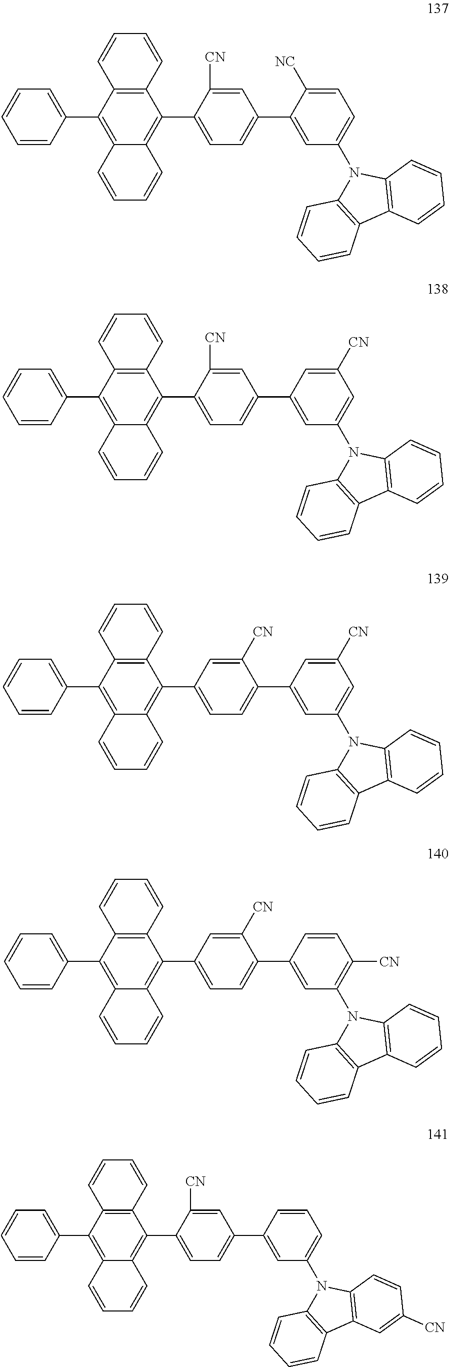 [SMILES: N#Cc1ccc(-n2c3ccccc3c3ccccc32)cc1-c1ccc(-c2c3ccccc3c(-c3ccccc3)c3ccccc23)c(C#N)c1 ; N#Cc1cc(-c2ccc(-c3c4ccccc4c(-c4ccccc4)c4ccccc34)c(C#N)c2)cc(-n2c3ccccc3c3ccccc32)c1 ; N#Cc1cc(-c2ccc(-c3c4ccccc4c(-c4ccccc4)c4ccccc34)cc2C#N)cc(-n2c3ccccc3c3ccccc32)c1 ; N#Cc1cc(-c2c3ccccc3c(-c3ccccc3)c3ccccc23)ccc1-c1ccc(C#N)c(-n2c3ccccc3c3ccccc32)c1 ; N#Cc1ccc2c(c1)c1ccccc1n2-c1cccc(-c2ccc(-c3c4ccccc4c(-c4ccccc4)c4ccccc34)c(C#N)c2)c1]

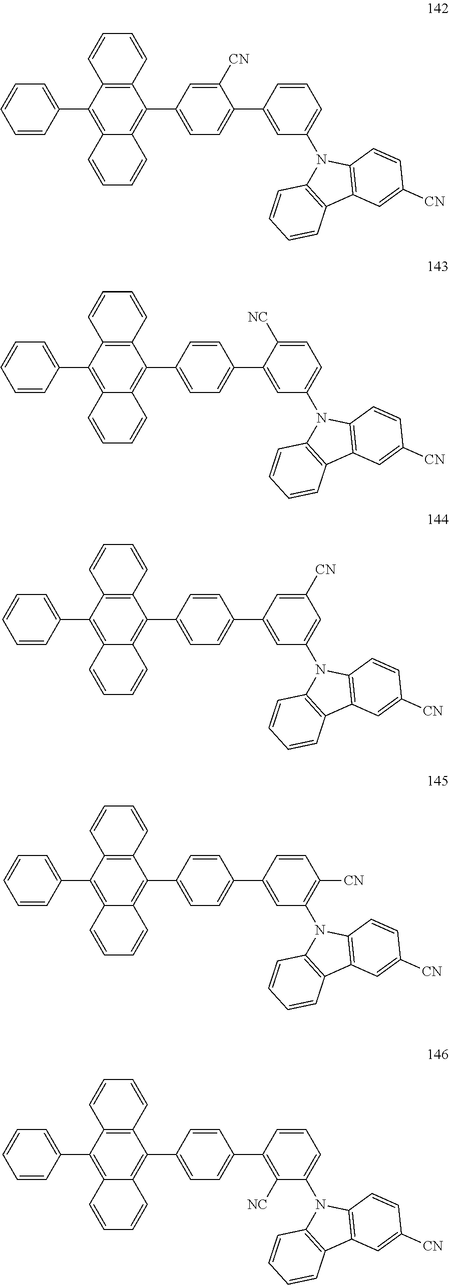 [SMILES: N#Cc1ccc2c(c1)c1ccccc1n2-c1cccc(-c2ccc(-c3c4ccccc4c(-c4ccccc4)c4ccccc34)cc2C#N)c1 ; N#Cc1ccc2c(c1)c1ccccc1n2-c1ccc(C#N)c(-c2ccc(-c3c4ccccc4c(-c4ccccc4)c4ccccc34)cc2)c1 ; N#Cc1cc(-c2ccc(-c3c4ccccc4c(-c4ccccc4)c4ccccc34)cc2)cc(-n2c3ccccc3c3cc(C#N)ccc32)c1 ; N#Cc1ccc2c(c1)c1ccccc1n2-c1cc(-c2ccc(-c3c4ccccc4c(-c4ccccc4)c4ccccc34)cc2)ccc1C#N ; N#Cc1ccc2c(c1)c1ccccc1n2-c1cccc(-c2ccc(-c3c4ccccc4c(-c4ccccc4)c4ccccc34)cc2)c1C#N]

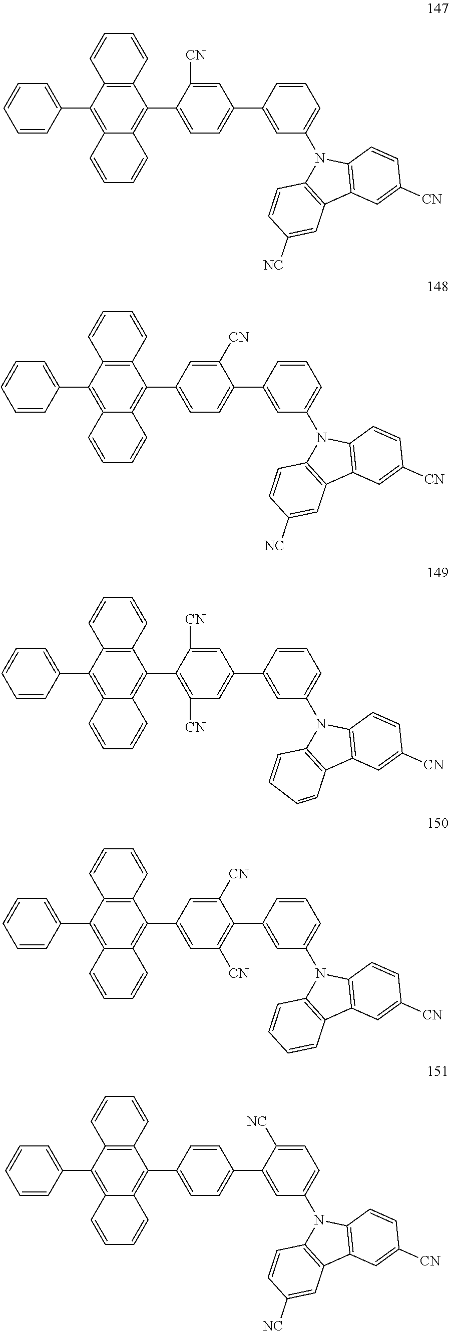 [SMILES: N#Cc1ccc2c(c1)c1cc(C#N)ccc1n2-c1cccc(-c2ccc(-c3c4ccccc4c(-c4ccccc4)c4ccccc34)c(C#N)c2)c1 ; N#Cc1ccc2c(c1)c1cc(C#N)ccc1n2-c1cccc(-c2ccc(-c3c4ccccc4c(-c4ccccc4)c4ccccc34)cc2C#N)c1 ; N#Cc1ccc2c(c1)c1ccccc1n2-c1cccc(-c2cc(C#N)c(-c3c4ccccc4c(-c4ccccc4)c4ccccc34)c(C#N)c2)c1 ; N#Cc1ccc2c(c1)c1ccccc1n2-c1cccc(-c2c(C#N)cc(-c3c4ccccc4c(-c4ccccc4)c4ccccc34)cc2C#N)c1 ; N#Cc1ccc2c(c1)c1cc(C#N)ccc1n2-c1ccc(C#N)c(-c2ccc(-c3c4ccccc4c(-c4ccccc4)c4ccccc34)cc2)c1]

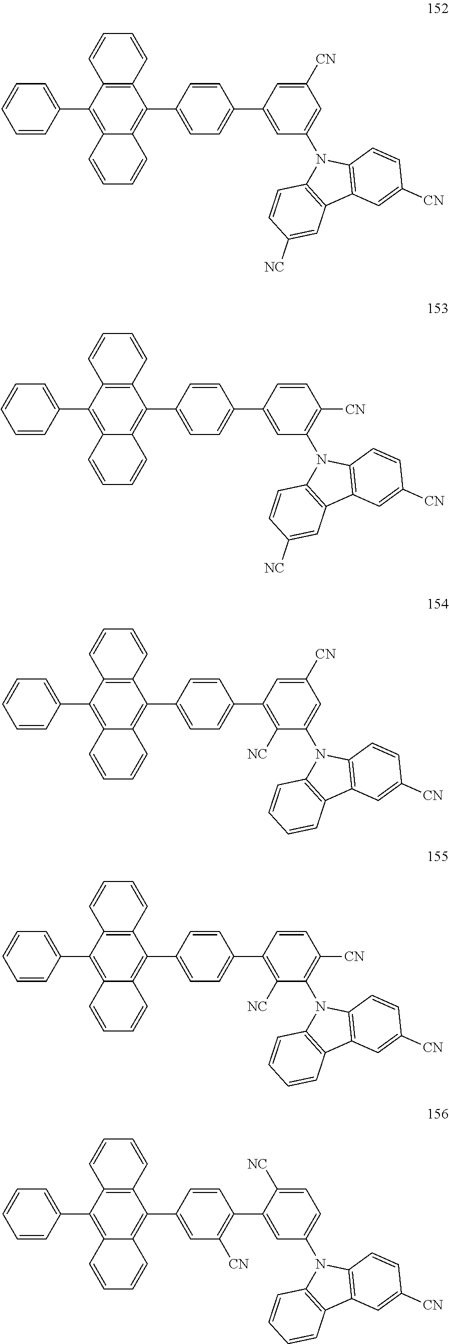 [SMILES: N#Cc1cc(-c2ccc(-c3c4ccccc4c(-c4ccccc4)c4ccccc34)cc2)cc(-n2c3ccc(C#N)cc3c3cc(C#N)ccc32)c1 ; N#Cc1ccc2c(c1)c1cc(C#N)ccc1n2-c1cc(-c2ccc(-c3c4ccccc4c(-c4ccccc4)c4ccccc34)cc2)ccc1C#N ; N#Cc1cc(-c2ccc(-c3c4ccccc4c(-c4ccccc4)c4ccccc34)cc2)c(C#N)c(-n2c3ccccc3c3cc(C#N)ccc32)c1 ; N#Cc1ccc2c(c1)c1ccccc1n2-c1c(C#N)ccc(-c2ccc(-c3c4ccccc4c(-c4ccccc4)c4ccccc34)cc2)c1C#N ; N#Cc1ccc2c(c1)c1ccccc1n2-c1ccc(C#N)c(-c2ccc(-c3c4ccccc4c(-c4ccccc4)c4ccccc34)cc2C#N)c1]

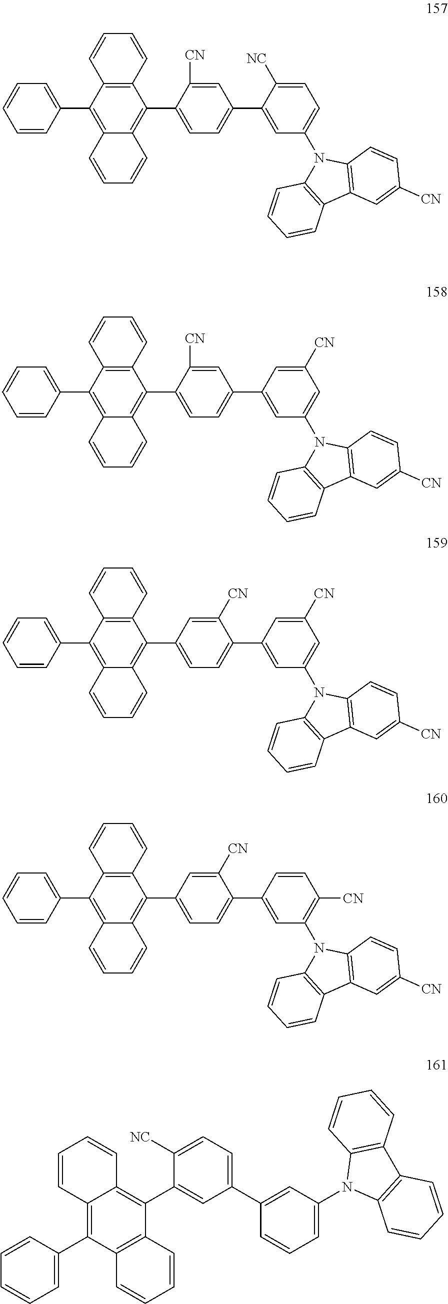 [SMILES: N#Cc1ccc2c(c1)c1ccccc1n2-c1ccc(C#N)c(-c2ccc(-c3c4ccccc4c(-c4ccccc4)c4ccccc34)c(C#N)c2)c1 ; N#Cc1cc(-c2ccc(-c3c4ccccc4c(-c4ccccc4)c4ccccc34)c(C#N)c2)cc(-n2c3ccccc3c3cc(C#N)ccc32)c1 ; N#Cc1cc(-c2ccc(-c3c4ccccc4c(-c4ccccc4)c4ccccc34)cc2C#N)cc(-n2c3ccccc3c3cc(C#N)ccc32)c1 ; N#Cc1ccc2c(c1)c1ccccc1n2-c1cc(-c2ccc(-c3c4ccccc4c(-c4ccccc4)c4ccccc34)cc2C#N)ccc1C#N ; N#Cc1ccc(-c2cccc(-n3c4ccccc4c4ccccc43)c2)cc1-c1c2ccccc2c(-c2ccccc2)c2ccccc12]

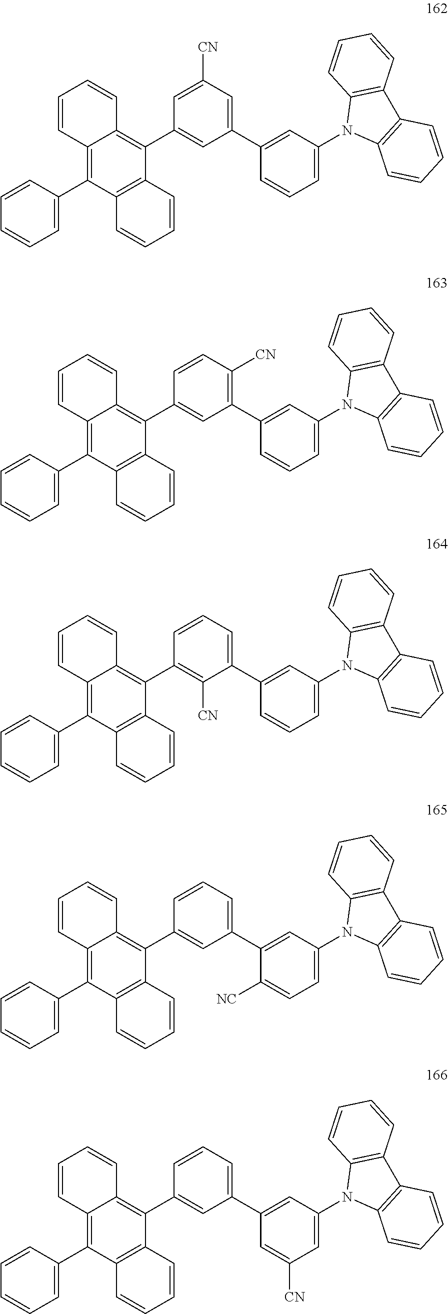 [SMILES: N#Cc1cc(-c2cccc(-n3c4ccccc4c4ccccc43)c2)cc(-c2c3ccccc3c(-c3ccccc3)c3ccccc23)c1 ; N#Cc1ccc(-c2c3ccccc3c(-c3ccccc3)c3ccccc23)cc1-c1cccc(-n2c3ccccc3c3ccccc32)c1 ; N#Cc1c(-c2cccc(-n3c4ccccc4c4ccccc43)c2)cccc1-c1c2ccccc2c(-c2ccccc2)c2ccccc12 ; N#Cc1ccc(-n2c3ccccc3c3ccccc32)cc1-c1cccc(-c2c3ccccc3c(-c3ccccc3)c3ccccc23)c1 ; N#Cc1cc(-c2cccc(-c3c4ccccc4c(-c4ccccc4)c4ccccc34)c2)cc(-n2c3ccccc3c3ccccc32)c1]

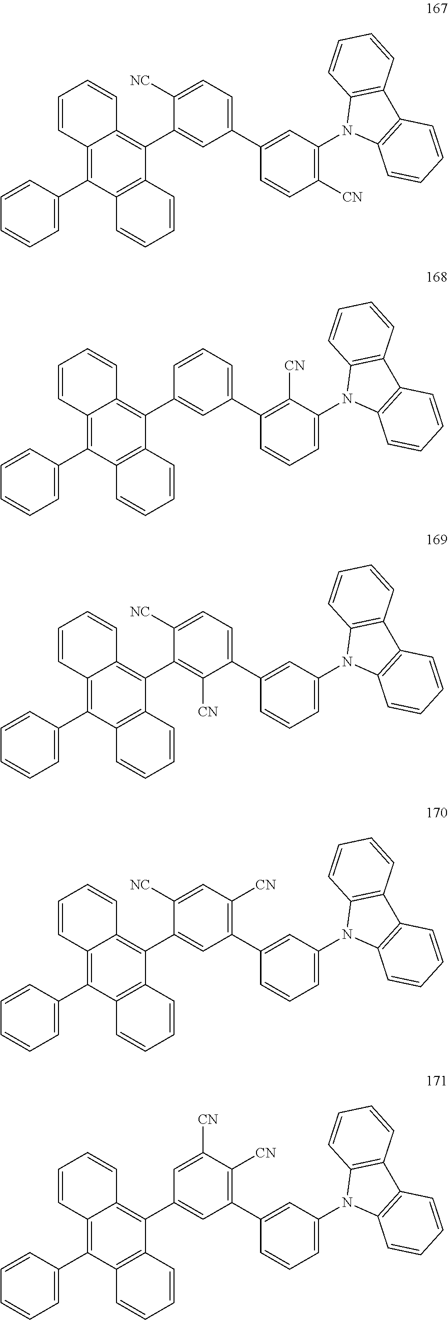 [SMILES: N#Cc1ccc(-c2ccc(C#N)c(-n3c4ccccc4c4ccccc43)c2)cc1-c1c2ccccc2c(-c2ccccc2)c2ccccc12 ; N#Cc1c(-c2cccc(-c3c4ccccc4c(-c4ccccc4)c4ccccc34)c2)cccc1-n1c2ccccc2c2ccccc21 ; N#Cc1ccc(-c2cccc(-n3c4ccccc4c4ccccc43)c2)c(C#N)c1-c1c2ccccc2c(-c2ccccc2)c2ccccc12 ; N#Cc1cc(C#N)c(-c2c3ccccc3c(-c3ccccc3)c3ccccc23)cc1-c1cccc(-n2c3ccccc3c3ccccc32)c1 ; N#Cc1cc(-c2c3ccccc3c(-c3ccccc3)c3ccccc23)cc(-c2cccc(-n3c4ccccc4c4ccccc43)c2)c1C#N]

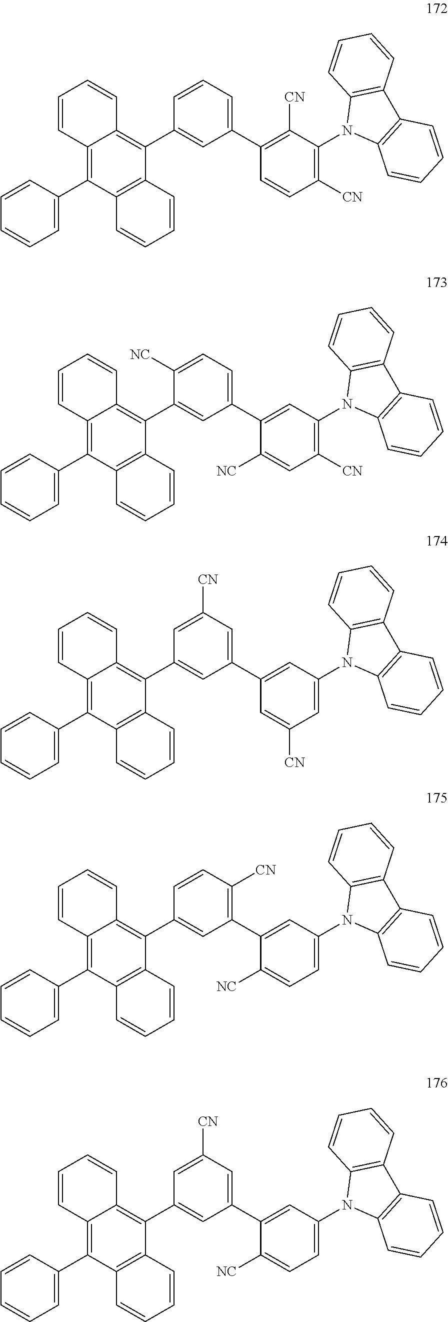 [SMILES: N#Cc1ccc(-c2cccc(-c3c4ccccc4c(-c4ccccc4)c4ccccc34)c2)c(C#N)c1-n1c2ccccc2c2ccccc21 ; N#Cc1cc(C#N)c(-n2c3ccccc3c3ccccc32)cc1-c1ccc(C#N)c(-c2c3ccccc3c(-c3ccccc3)c3ccccc23)c1 ; N#Cc1cc(-c2cc(C#N)cc(-n3c4ccccc4c4ccccc43)c2)cc(-c2c3ccccc3c(-c3ccccc3)c3ccccc23)c1 ; N#Cc1ccc(-c2c3ccccc3c(-c3ccccc3)c3ccccc23)cc1-c1cc(-n2c3ccccc3c3ccccc32)ccc1C#N ; N#Cc1cc(-c2cc(-n3c4ccccc4c4ccccc43)ccc2C#N)cc(-c2c3ccccc3c(-c3ccccc3)c3ccccc23)c1]

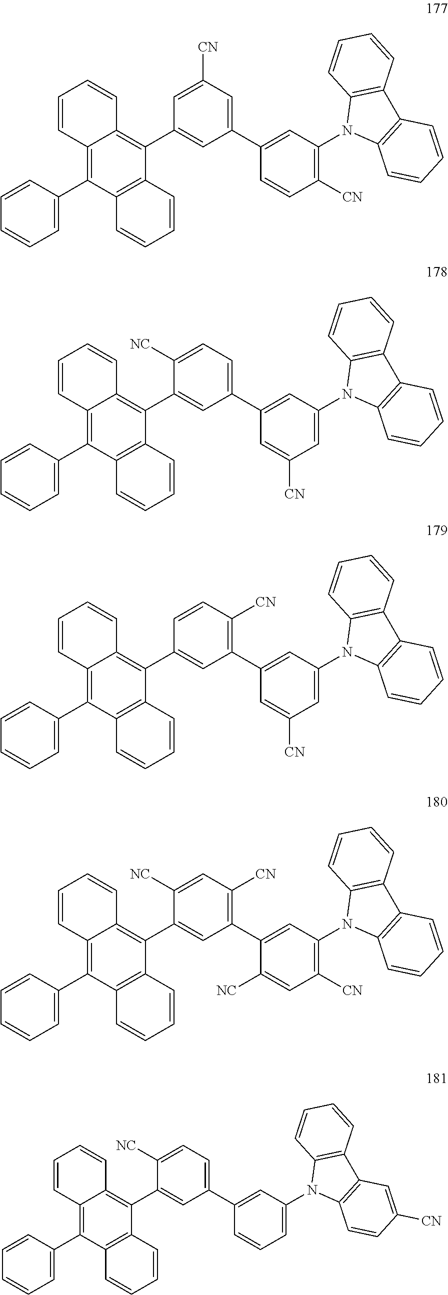 [SMILES: N#Cc1cc(-c2ccc(C#N)c(-n3c4ccccc4c4ccccc43)c2)cc(-c2c3ccccc3c(-c3ccccc3)c3ccccc23)c1 ; N#Cc1cc(-c2ccc(C#N)c(-c3c4ccccc4c(-c4ccccc4)c4ccccc34)c2)cc(-n2c3ccccc3c3ccccc32)c1 ; N#Cc1cc(-c2cc(-c3c4ccccc4c(-c4ccccc4)c4ccccc34)ccc2C#N)cc(-n2c3ccccc3c3ccccc32)c1 ; N#Cc1cc(C#N)c(-c2c3ccccc3c(-c3ccccc3)c3ccccc23)cc1-c1cc(-n2c3ccccc3c3ccccc32)c(C#N)cc1C#N ; N#Cc1ccc2c(c1)c1ccccc1n2-c1cccc(-c2ccc(C#N)c(-c3c4ccccc4c(-c4ccccc4)c4ccccc34)c2)c1]

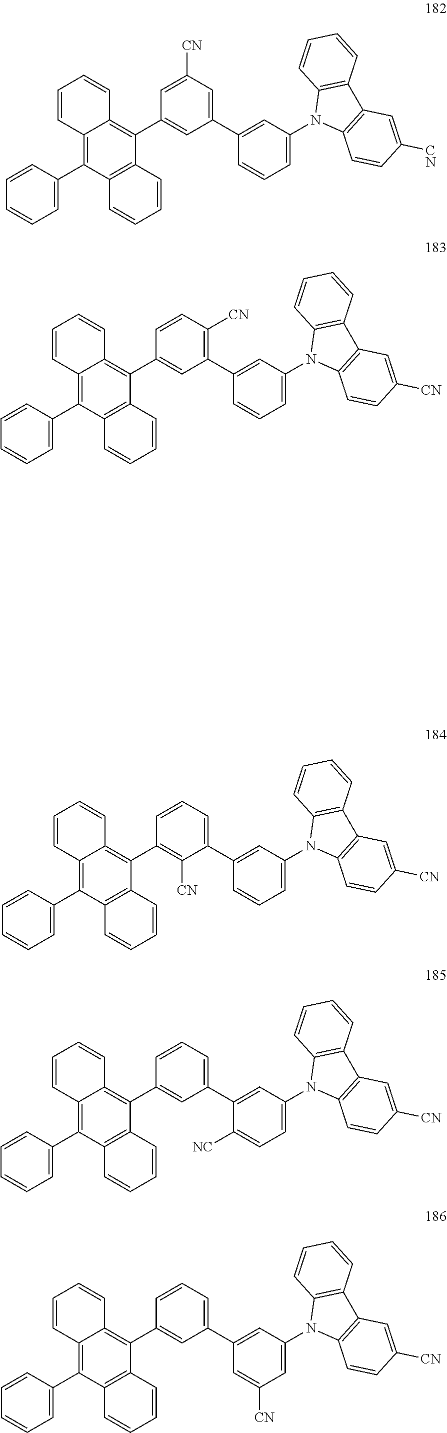 [SMILES: N#Cc1cc(-c2cccc(-n3c4ccccc4c4cc(C#N)ccc43)c2)cc(-c2c3ccccc3c(-c3ccccc3)c3ccccc23)c1 ; N#Cc1ccc2c(c1)c1ccccc1n2-c1cccc(-c2cc(-c3c4ccccc4c(-c4ccccc4)c4ccccc34)ccc2C#N)c1 ; N#Cc1ccc2c(c1)c1ccccc1n2-c1cccc(-c2cccc(-c3c4ccccc4c(-c4ccccc4)c4ccccc34)c2C#N)c1 ; N#Cc1ccc2c(c1)c1ccccc1n2-c1ccc(C#N)c(-c2cccc(-c3c4ccccc4c(-c4ccccc4)c4ccccc34)c2)c1 ; N#Cc1cc(-c2cccc(-c3c4ccccc4c(-c4ccccc4)c4ccccc34)c2)cc(-n2c3ccccc3c3cc(C#N)ccc32)c1]

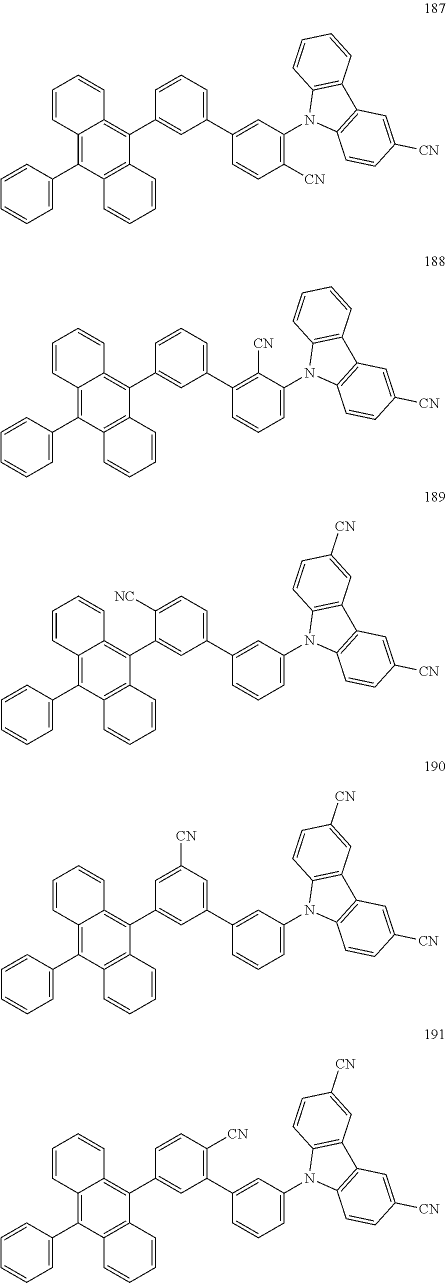 [SMILES: N#Cc1ccc2c(c1)c1ccccc1n2-c1cc(-c2cccc(-c3c4ccccc4c(-c4ccccc4)c4ccccc34)c2)ccc1C#N ; N#Cc1ccc2c(c1)c1ccccc1n2-c1cccc(-c2cccc(-c3c4ccccc4c(-c4ccccc4)c4ccccc34)c2)c1C#N ; N#Cc1ccc2c(c1)c1cc(C#N)ccc1n2-c1cccc(-c2ccc(C#N)c(-c3c4ccccc4c(-c4ccccc4)c4ccccc34)c2)c1 ; N#Cc1cc(-c2cccc(-n3c4ccc(C#N)cc4c4cc(C#N)ccc43)c2)cc(-c2c3ccccc3c(-c3ccccc3)c3ccccc23)c1 ; N#Cc1ccc2c(c1)c1cc(C#N)ccc1n2-c1cccc(-c2cc(-c3c4ccccc4c(-c4ccccc4)c4ccccc34)ccc2C#N)c1]

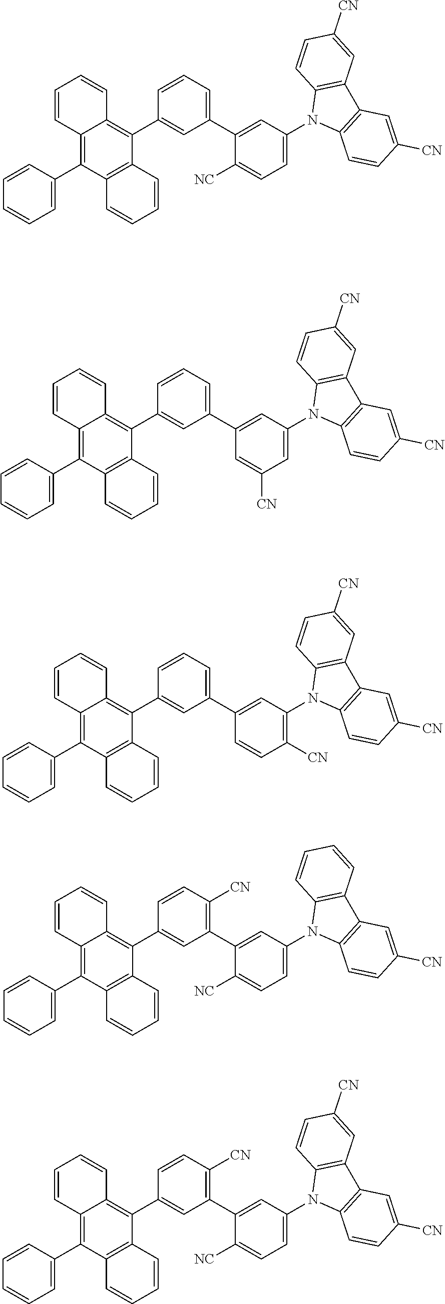 [SMILES: N#Cc1ccc2c(c1)c1cc(C#N)ccc1n2-c1ccc(C#N)c(-c2cccc(-c3c4ccccc4c(-c4ccccc4)c4ccccc34)c2)c1 ; N#Cc1cc(-c2cccc(-c3c4ccccc4c(-c4ccccc4)c4ccccc34)c2)cc(-n2c3ccc(C#N)cc3c3cc(C#N)ccc32)c1 ; N#Cc1ccc2c(c1)c1cc(C#N)ccc1n2-c1cc(-c2cccc(-c3c4ccccc4c(-c4ccccc4)c4ccccc34)c2)ccc1C#N ; N#Cc1ccc2c(c1)c1ccccc1n2-c1ccc(C#N)c(-c2cc(-c3c4ccccc4c(-c4ccccc4)c4ccccc34)ccc2C#N)c1 ; N#Cc1ccc2c(c1)c1cc(C#N)ccc1n2-c1ccc(C#N)c(-c2cc(-c3c4ccccc4c(-c4ccccc4)c4ccccc34)ccc2C#N)c1]

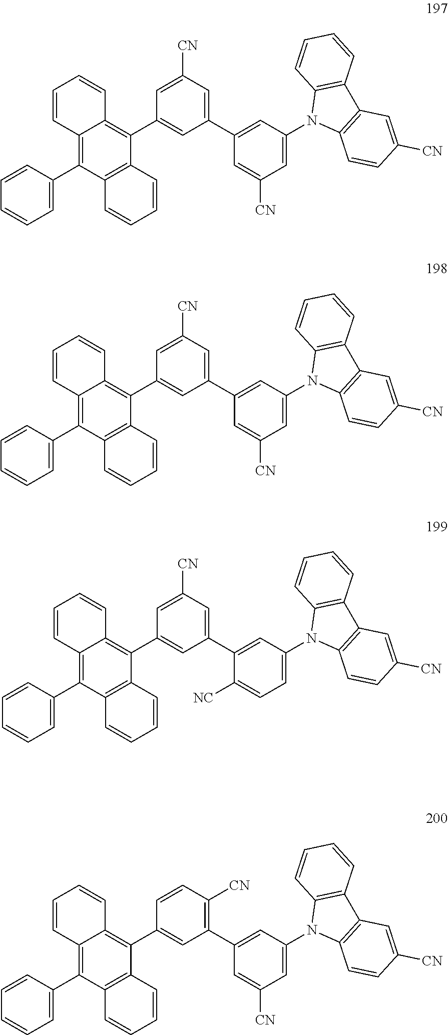 [SMILES: N#Cc1cc(-c2cc(C#N)cc(-n3c4ccccc4c4cc(C#N)ccc43)c2)cc(-c2c3ccccc3c(-c3ccccc3)c3ccccc23)c1 ; N#Cc1cc(-c2cc(C#N)cc(-n3c4ccccc4c4cc(C#N)ccc43)c2)cc(-c2c3ccccc3c(-c3ccccc3)c3ccccc23)c1 ; N#Cc1cc(-c2cc(-n3c4ccccc4c4cc(C#N)ccc43)ccc2C#N)cc(-c2c3ccccc3c(-c3ccccc3)c3ccccc23)c1 ; N#Cc1cc(-c2cc(-c3c4ccccc4c(-c4ccccc4)c4ccccc34)ccc2C#N)cc(-n2c3ccccc3c3cc(C#N)ccc32)c1]

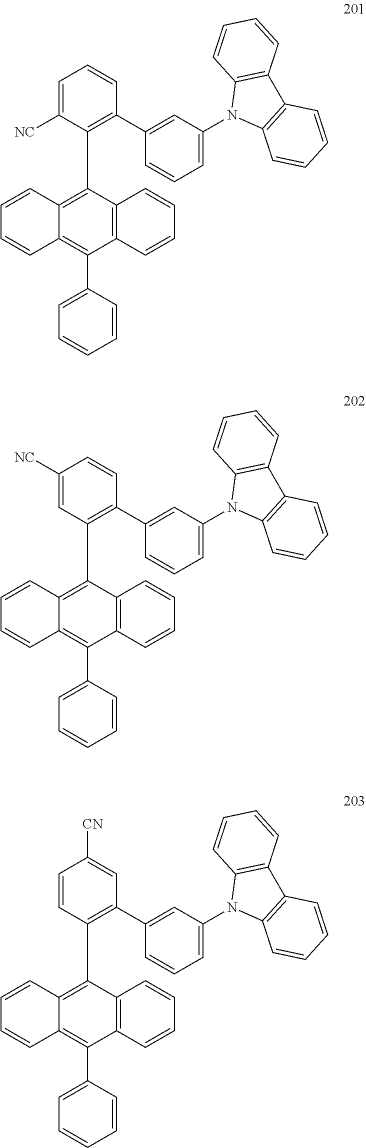 [SMILES: N#Cc1cccc(-c2cccc(-n3c4ccccc4c4ccccc43)c2)c1-c1c2ccccc2c(-c2ccccc2)c2ccccc12 ; N#Cc1ccc(-c2cccc(-n3c4ccccc4c4ccccc43)c2)c(-c2c3ccccc3c(-c3ccccc3)c3ccccc23)c1 ; N#Cc1ccc(-c2c3ccccc3c(-c3ccccc3)c3ccccc23)c(-c2cccc(-n3c4ccccc4c4ccccc43)c2)c1]

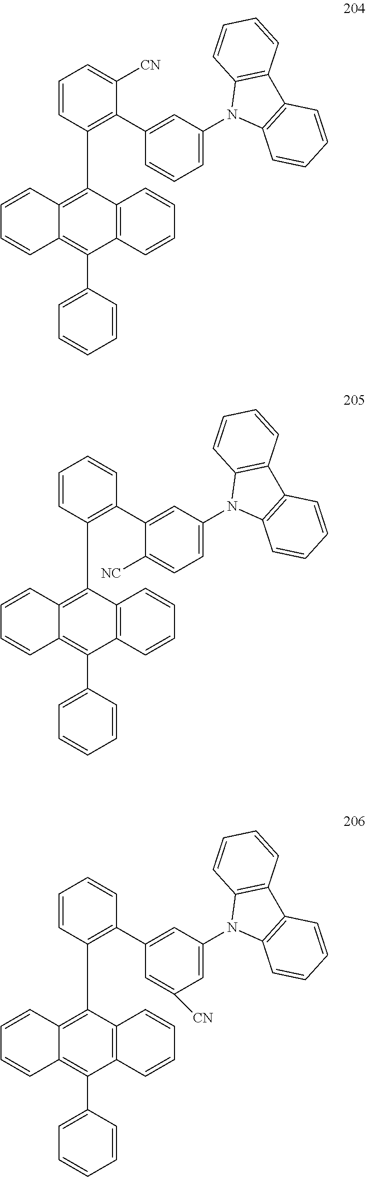 [SMILES: N#Cc1cccc(-c2c3ccccc3c(-c3ccccc3)c3ccccc23)c1-c1cccc(-n2c3ccccc3c3ccccc32)c1 ; N#Cc1ccc(-n2c3ccccc3c3ccccc32)cc1-c1ccccc1-c1c2ccccc2c(-c2ccccc2)c2ccccc12 ; N#Cc1cc(-c2ccccc2-c2c3ccccc3c(-c3ccccc3)c3ccccc23)cc(-n2c3ccccc3c3ccccc32)c1]

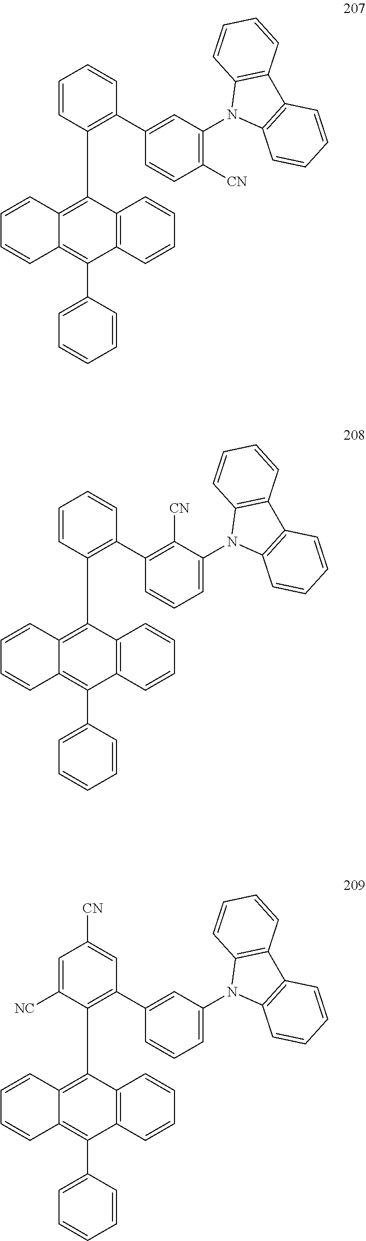 [SMILES: N#Cc1ccc(-c2ccccc2-c2c3ccccc3c(-c3ccccc3)c3ccccc23)cc1-n1c2ccccc2c2ccccc21 ; N#Cc1c(-c2ccccc2-c2c3ccccc3c(-c3ccccc3)c3ccccc23)cccc1-n1c2ccccc2c2ccccc21 ; N#Cc1cc(C#N)c(-c2c3ccccc3c(-c3ccccc3)c3ccccc23)c(-c2cccc(-n3c4ccccc4c4ccccc43)c2)c1]

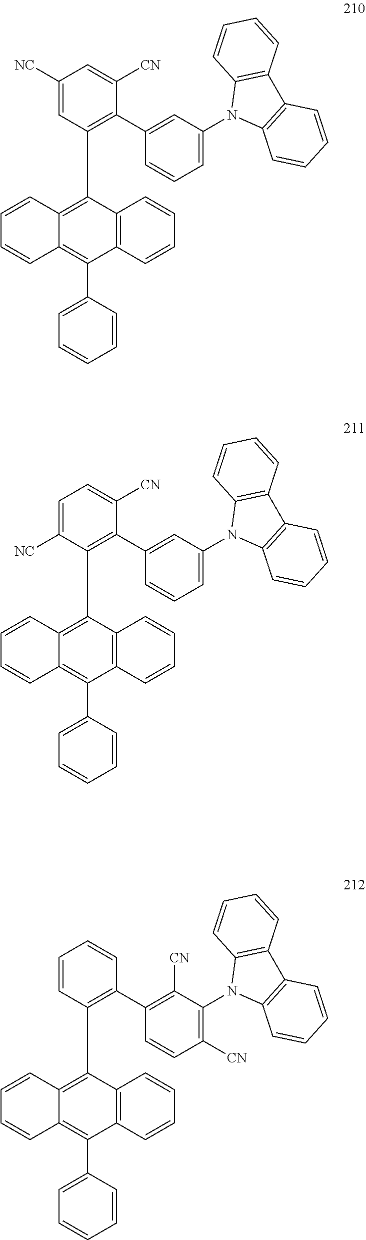 [SMILES: N#Cc1cc(C#N)c(-c2cccc(-n3c4ccccc4c4ccccc43)c2)c(-c2c3ccccc3c(-c3ccccc3)c3ccccc23)c1 ; N#Cc1ccc(C#N)c(-c2c3ccccc3c(-c3ccccc3)c3ccccc23)c1-c1cccc(-n2c3ccccc3c3ccccc32)c1 ; N#Cc1ccc(-c2ccccc2-c2c3ccccc3c(-c3ccccc3)c3ccccc23)c(C#N)c1-n1c2ccccc2c2ccccc21]

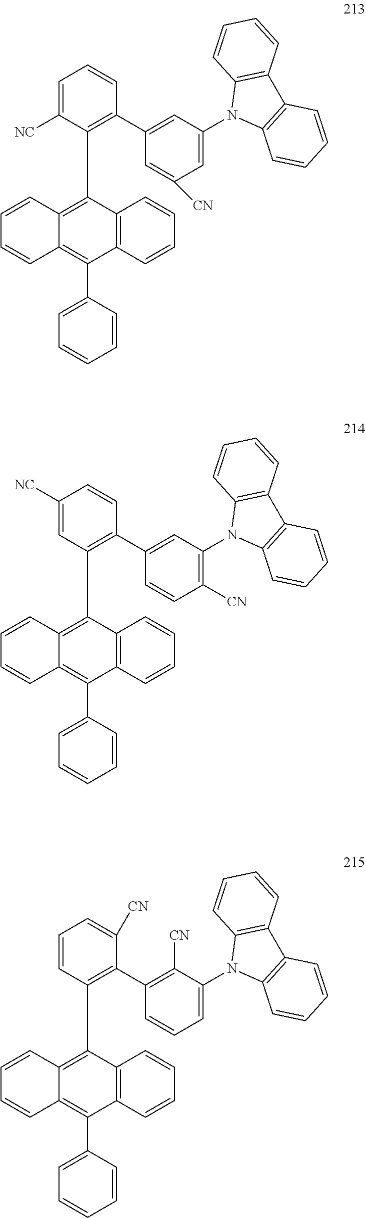 [SMILES: N#Cc1cc(-c2cccc(C#N)c2-c2c3ccccc3c(-c3ccccc3)c3ccccc23)cc(-n2c3ccccc3c3ccccc32)c1 ; N#Cc1ccc(-c2ccc(C#N)c(-n3c4ccccc4c4ccccc43)c2)c(-c2c3ccccc3c(-c3ccccc3)c3ccccc23)c1 ; N#Cc1cccc(-c2c3ccccc3c(-c3ccccc3)c3ccccc23)c1-c1cccc(-n2c3ccccc3c3ccccc32)c1C#N]

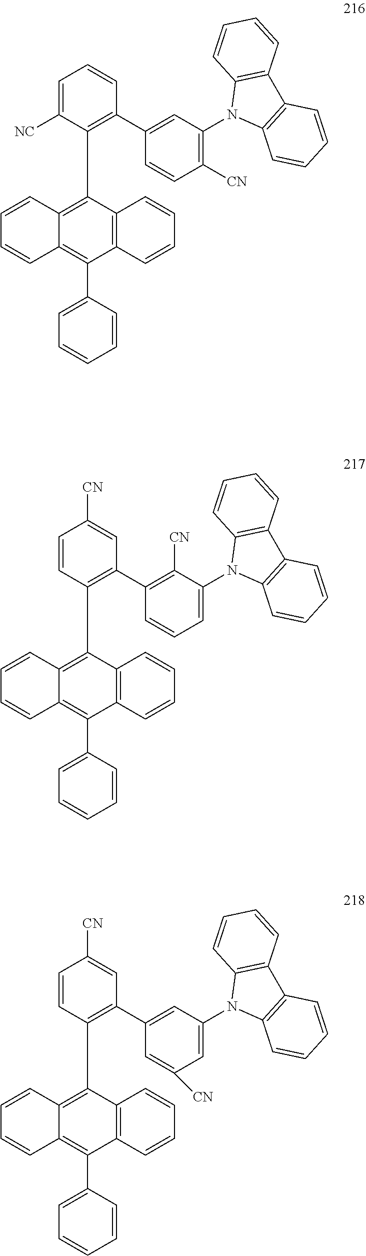 [SMILES: N#Cc1ccc(-c2cccc(C#N)c2-c2c3ccccc3c(-c3ccccc3)c3ccccc23)cc1-n1c2ccccc2c2ccccc21 ; N#Cc1ccc(-c2c3ccccc3c(-c3ccccc3)c3ccccc23)c(-c2cccc(-n3c4ccccc4c4ccccc43)c2C#N)c1 ; N#Cc1cc(-c2cc(C#N)ccc2-c2c3ccccc3c(-c3ccccc3)c3ccccc23)cc(-n2c3ccccc3c3ccccc32)c1]

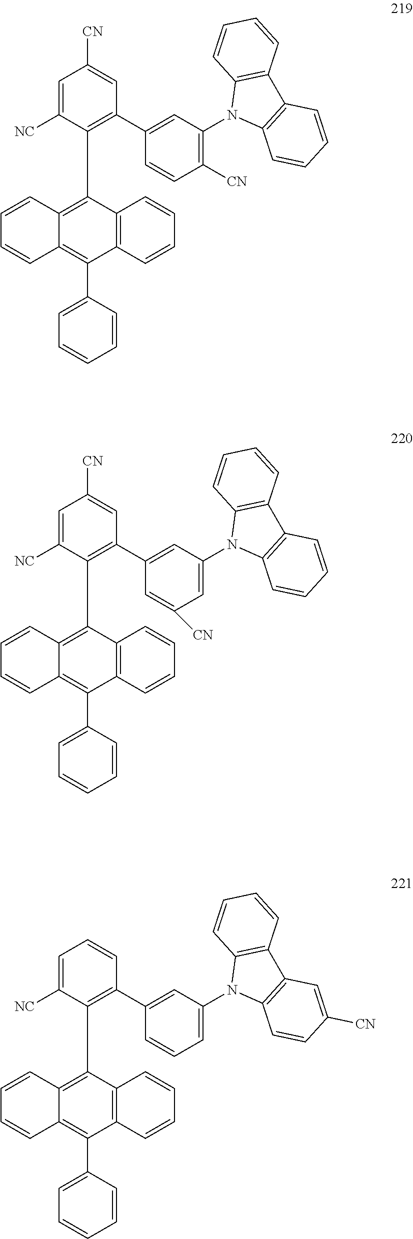 [SMILES: N#Cc1cc(C#N)c(-c2c3ccccc3c(-c3ccccc3)c3ccccc23)c(-c2ccc(C#N)c(-n3c4ccccc4c4ccccc43)c2)c1 ; N#Cc1cc(-c2cc(C#N)cc(C#N)c2-c2c3ccccc3c(-c3ccccc3)c3ccccc23)cc(-n2c3ccccc3c3ccccc32)c1 ; N#Cc1ccc2c(c1)c1ccccc1n2-c1cccc(-c2cccc(C#N)c2-c2c3ccccc3c(-c3ccccc3)c3ccccc23)c1]

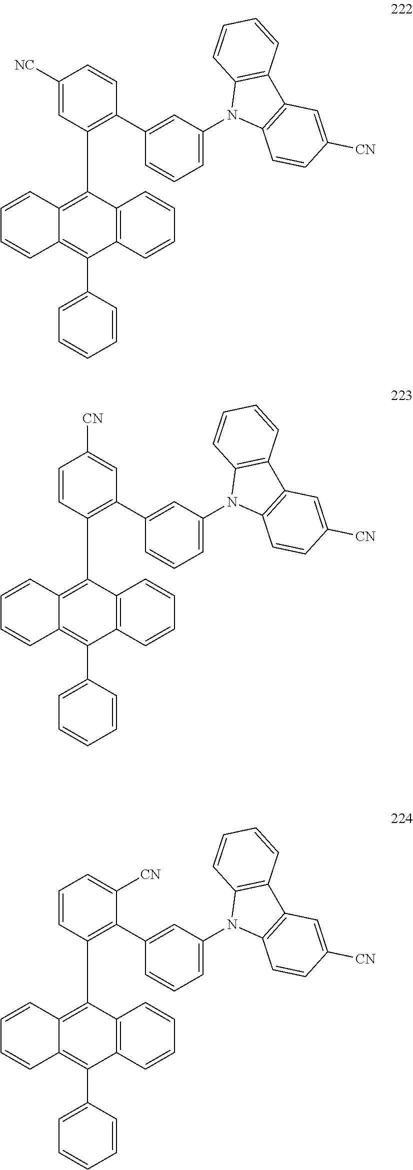 [SMILES: N#Cc1ccc(-c2cccc(-n3c4ccccc4c4cc(C#N)ccc43)c2)c(-c2c3ccccc3c(-c3ccccc3)c3ccccc23)c1 ; N#Cc1ccc(-c2c3ccccc3c(-c3ccccc3)c3ccccc23)c(-c2cccc(-n3c4ccccc4c4cc(C#N)ccc43)c2)c1 ; N#Cc1ccc2c(c1)c1ccccc1n2-c1cccc(-c2c(C#N)cccc2-c2c3ccccc3c(-c3ccccc3)c3ccccc23)c1]

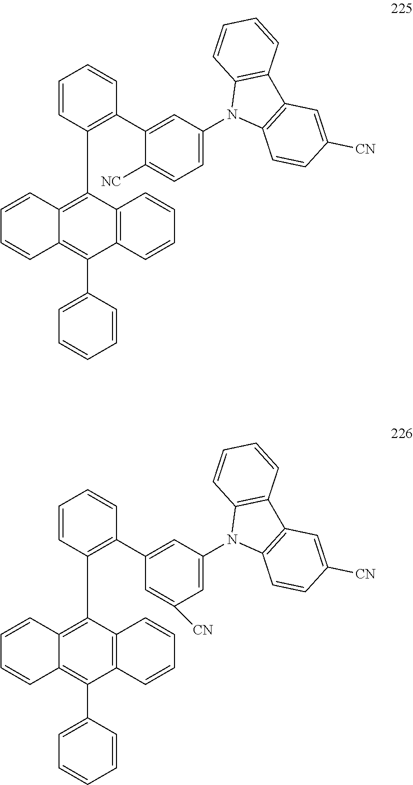 [SMILES: N#Cc1ccc2c(c1)c1ccccc1n2-c1ccc(C#N)c(-c2ccccc2-c2c3ccccc3c(-c3ccccc3)c3ccccc23)c1 ; N#Cc1cc(-c2ccccc2-c2c3ccccc3c(-c3ccccc3)c3ccccc23)cc(-n2c3ccccc3c3cc(C#N)ccc32)c1]

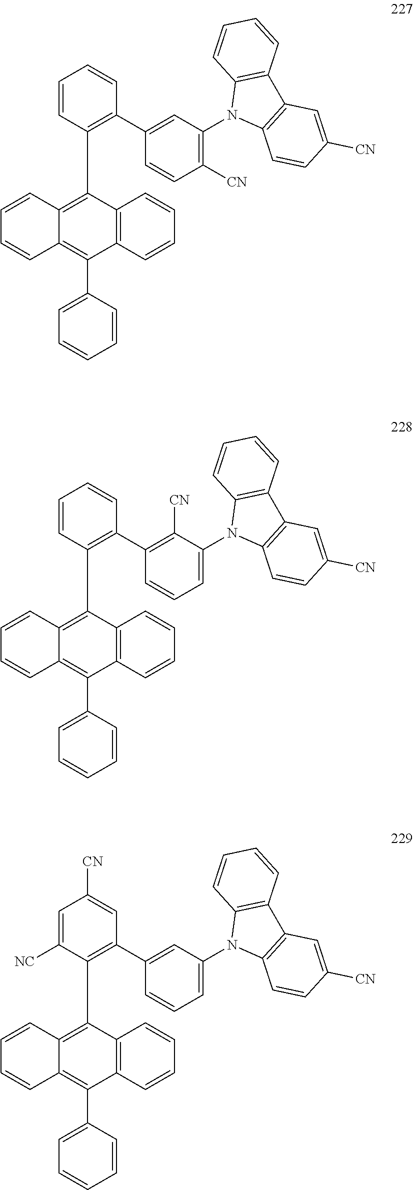 [SMILES: N#Cc1ccc2c(c1)c1ccccc1n2-c1cc(-c2ccccc2-c2c3ccccc3c(-c3ccccc3)c3ccccc23)ccc1C#N ; N#Cc1ccc2c(c1)c1ccccc1n2-c1cccc(-c2ccccc2-c2c3ccccc3c(-c3ccccc3)c3ccccc23)c1C#N ; N#Cc1cc(C#N)c(-c2c3ccccc3c(-c3ccccc3)c3ccccc23)c(-c2cccc(-n3c4ccccc4c4cc(C#N)ccc43)c2)c1]

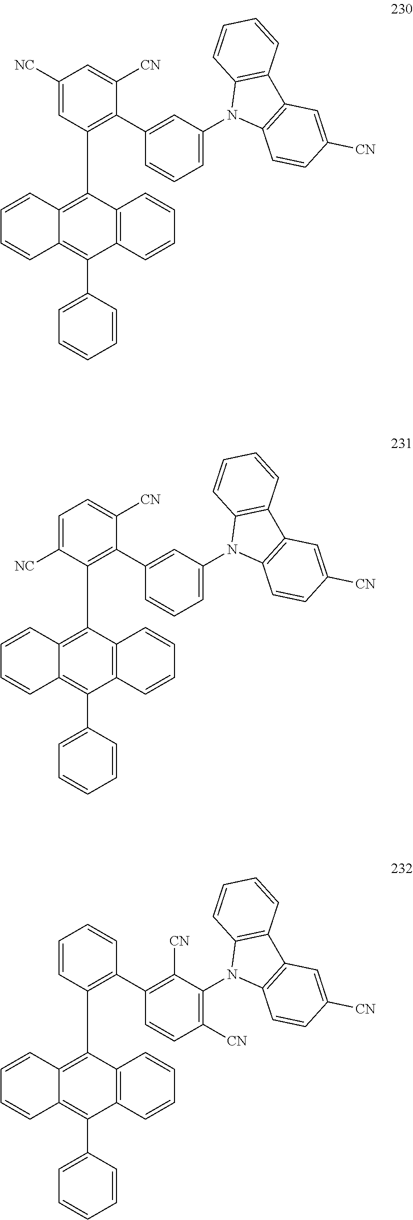 [SMILES: N#Cc1cc(C#N)c(-c2cccc(-n3c4ccccc4c4cc(C#N)ccc43)c2)c(-c2c3ccccc3c(-c3ccccc3)c3ccccc23)c1 ; N#Cc1ccc2c(c1)c1ccccc1n2-c1cccc(-c2c(C#N)ccc(C#N)c2-c2c3ccccc3c(-c3ccccc3)c3ccccc23)c1 ; N#Cc1ccc2c(c1)c1ccccc1n2-c1c(C#N)ccc(-c2ccccc2-c2c3ccccc3c(-c3ccccc3)c3ccccc23)c1C#N]

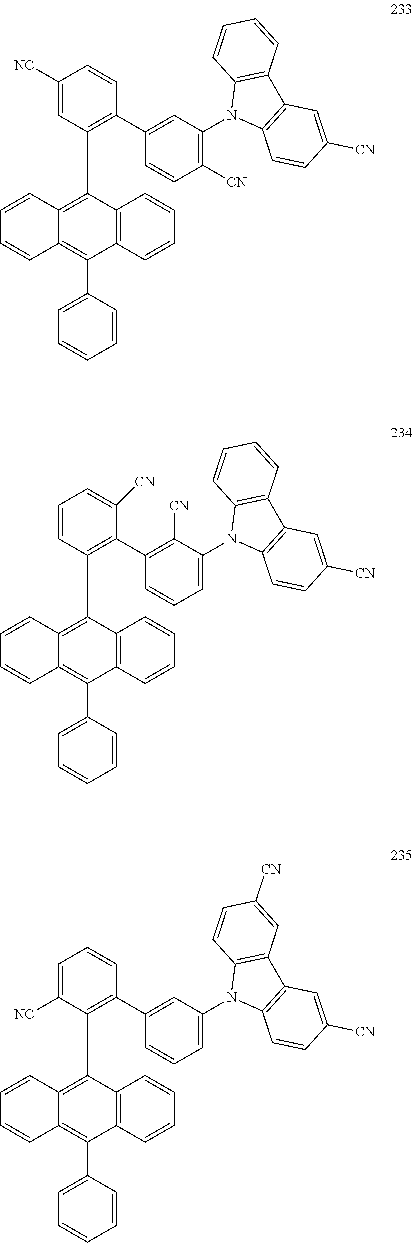 [SMILES: N#Cc1ccc(-c2ccc(C#N)c(-n3c4ccccc4c4cc(C#N)ccc43)c2)c(-c2c3ccccc3c(-c3ccccc3)c3ccccc23)c1 ; N#Cc1ccc2c(c1)c1ccccc1n2-c1cccc(-c2c(C#N)cccc2-c2c3ccccc3c(-c3ccccc3)c3ccccc23)c1C#N ; N#Cc1ccc2c(c1)c1cc(C#N)ccc1n2-c1cccc(-c2cccc(C#N)c2-c2c3ccccc3c(-c3ccccc3)c3ccccc23)c1]

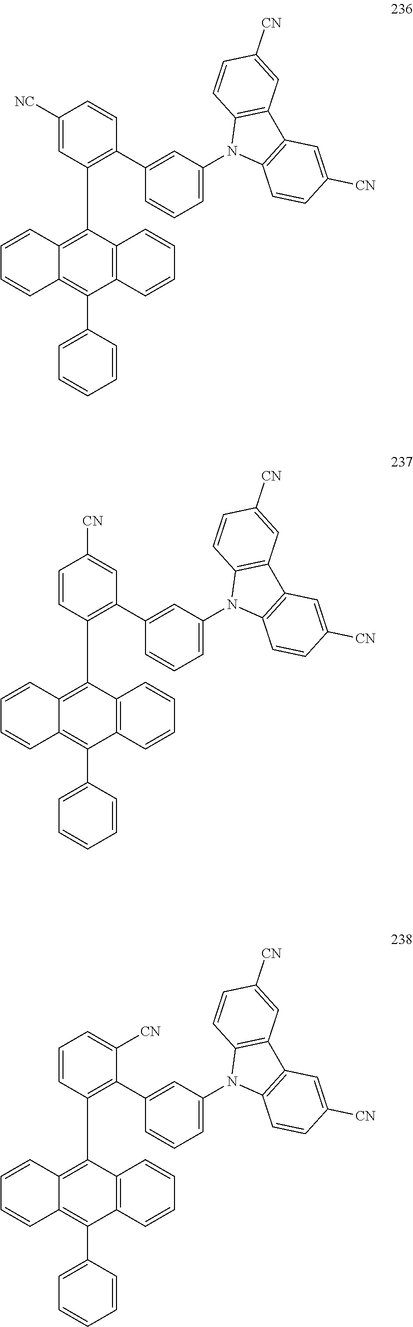 [SMILES: N#Cc1ccc(-c2cccc(-n3c4ccc(C#N)cc4c4cc(C#N)ccc43)c2)c(-c2c3ccccc3c(-c3ccccc3)c3ccccc23)c1 ; N#Cc1ccc(-c2c3ccccc3c(-c3ccccc3)c3ccccc23)c(-c2cccc(-n3c4ccc(C#N)cc4c4cc(C#N)ccc43)c2)c1 ; N#Cc1ccc2c(c1)c1cc(C#N)ccc1n2-c1cccc(-c2c(C#N)cccc2-c2c3ccccc3c(-c3ccccc3)c3ccccc23)c1]

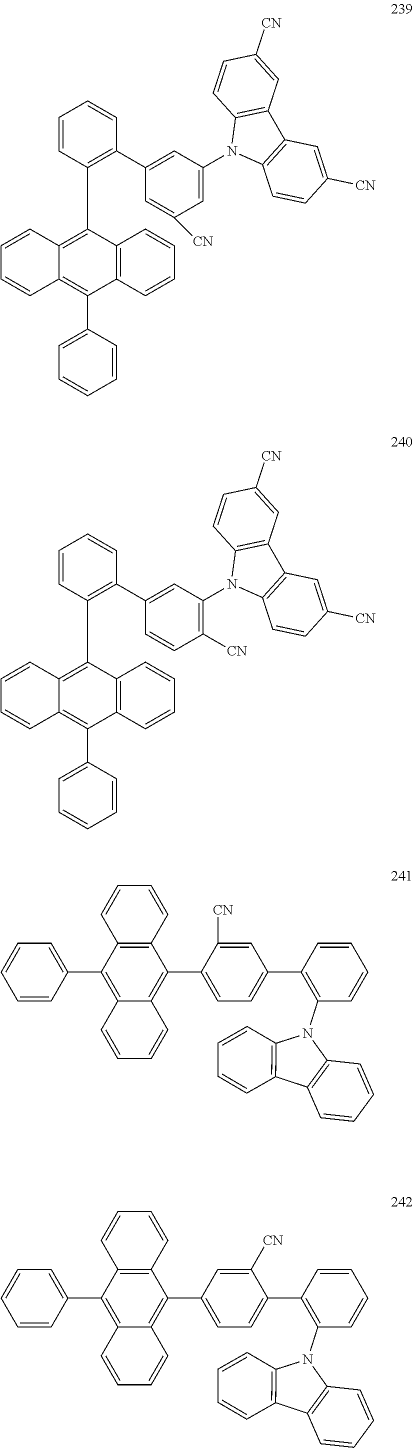 [SMILES: N#Cc1cc(-c2ccccc2-c2c3ccccc3c(-c3ccccc3)c3ccccc23)cc(-n2c3ccc(C#N)cc3c3cc(C#N)ccc32)c1 ; N#Cc1ccc2c(c1)c1cc(C#N)ccc1n2-c1cc(-c2ccccc2-c2c3ccccc3c(-c3ccccc3)c3ccccc23)ccc1C#N ; N#Cc1cc(-c2ccccc2-n2c3ccccc3c3ccccc32)ccc1-c1c2ccccc2c(-c2ccccc2)c2ccccc12 ; N#Cc1cc(-c2c3ccccc3c(-c3ccccc3)c3ccccc23)ccc1-c1ccccc1-n1c2ccccc2c2ccccc21]

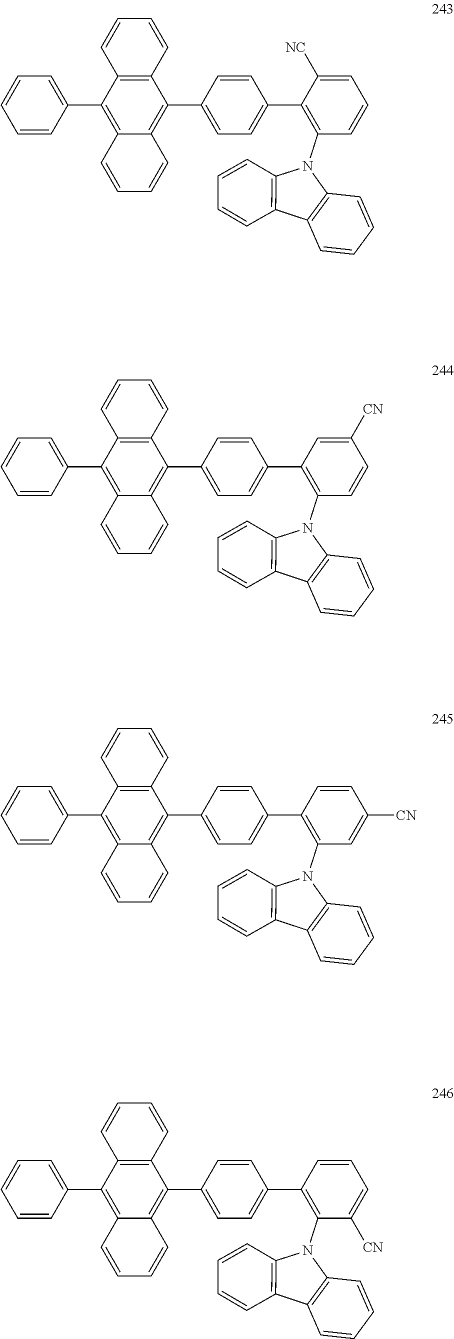 [SMILES: N#Cc1cccc(-n2c3ccccc3c3ccccc32)c1-c1ccc(-c2c3ccccc3c(-c3ccccc3)c3ccccc23)cc1 ; N#Cc1ccc(-n2c3ccccc3c3ccccc32)c(-c2ccc(-c3c4ccccc4c(-c4ccccc4)c4ccccc34)cc2)c1 ; N#Cc1ccc(-c2ccc(-c3c4ccccc4c(-c4ccccc4)c4ccccc34)cc2)c(-n2c3ccccc3c3ccccc32)c1 ; N#Cc1cccc(-c2ccc(-c3c4ccccc4c(-c4ccccc4)c4ccccc34)cc2)c1-n1c2ccccc2c2ccccc21]

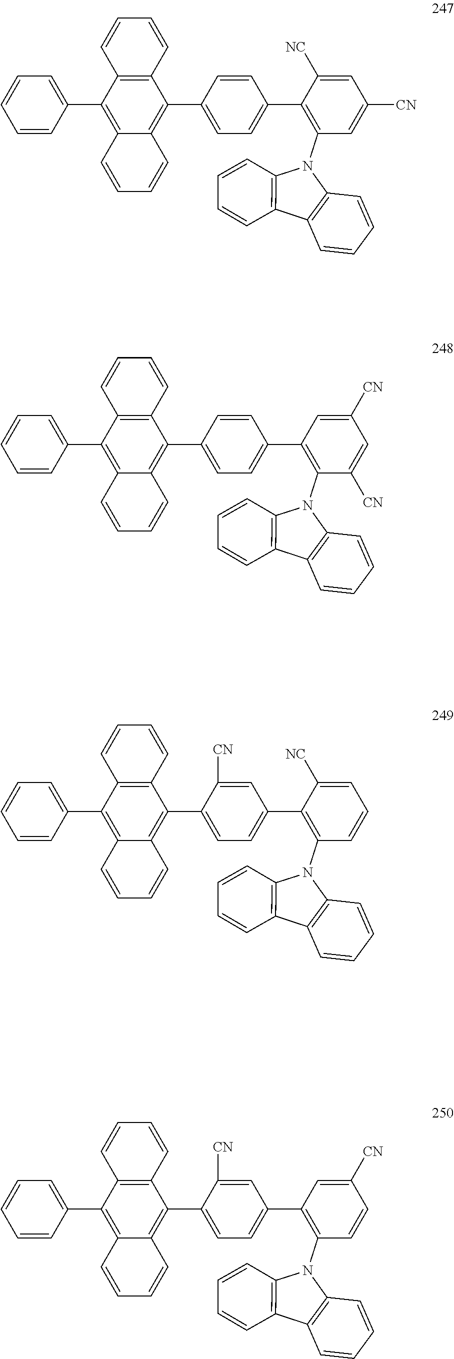 [SMILES: N#Cc1cc(C#N)c(-c2ccc(-c3c4ccccc4c(-c4ccccc4)c4ccccc34)cc2)c(-n2c3ccccc3c3ccccc32)c1 ; N#Cc1cc(C#N)c(-n2c3ccccc3c3ccccc32)c(-c2ccc(-c3c4ccccc4c(-c4ccccc4)c4ccccc34)cc2)c1 ; N#Cc1cc(-c2c(C#N)cccc2-n2c3ccccc3c3ccccc32)ccc1-c1c2ccccc2c(-c2ccccc2)c2ccccc12 ; N#Cc1ccc(-n2c3ccccc3c3ccccc32)c(-c2ccc(-c3c4ccccc4c(-c4ccccc4)c4ccccc34)c(C#N)c2)c1]

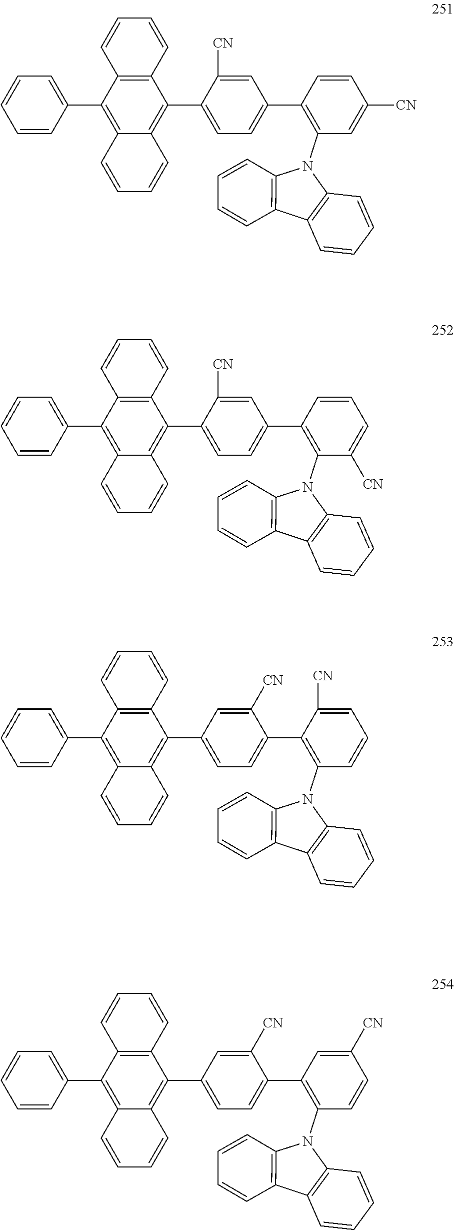 [SMILES: N#Cc1ccc(-c2ccc(-c3c4ccccc4c(-c4ccccc4)c4ccccc34)c(C#N)c2)c(-n2c3ccccc3c3ccccc32)c1 ; N#Cc1cc(-c2cccc(C#N)c2-n2c3ccccc3c3ccccc32)ccc1-c1c2ccccc2c(-c2ccccc2)c2ccccc12 ; N#Cc1cc(-c2c3ccccc3c(-c3ccccc3)c3ccccc23)ccc1-c1c(C#N)cccc1-n1c2ccccc2c2ccccc21 ; N#Cc1ccc(-n2c3ccccc3c3ccccc32)c(-c2ccc(-c3c4ccccc4c(-c4ccccc4)c4ccccc34)cc2C#N)c1]

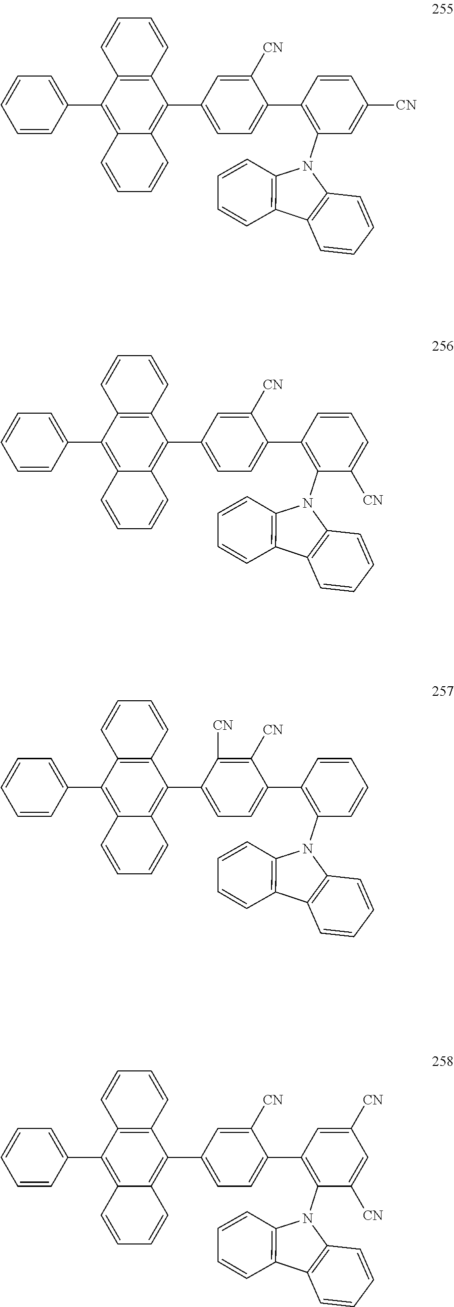 [SMILES: N#Cc1ccc(-c2ccc(-c3c4ccccc4c(-c4ccccc4)c4ccccc34)cc2C#N)c(-n2c3ccccc3c3ccccc32)c1 ; N#Cc1cc(-c2c3ccccc3c(-c3ccccc3)c3ccccc23)ccc1-c1cccc(C#N)c1-n1c2ccccc2c2ccccc21 ; N#Cc1c(-c2ccccc2-n2c3ccccc3c3ccccc32)ccc(-c2c3ccccc3c(-c3ccccc3)c3ccccc23)c1C#N ; N#Cc1cc(C#N)c(-n2c3ccccc3c3ccccc32)c(-c2ccc(-c3c4ccccc4c(-c4ccccc4)c4ccccc34)cc2C#N)c1]

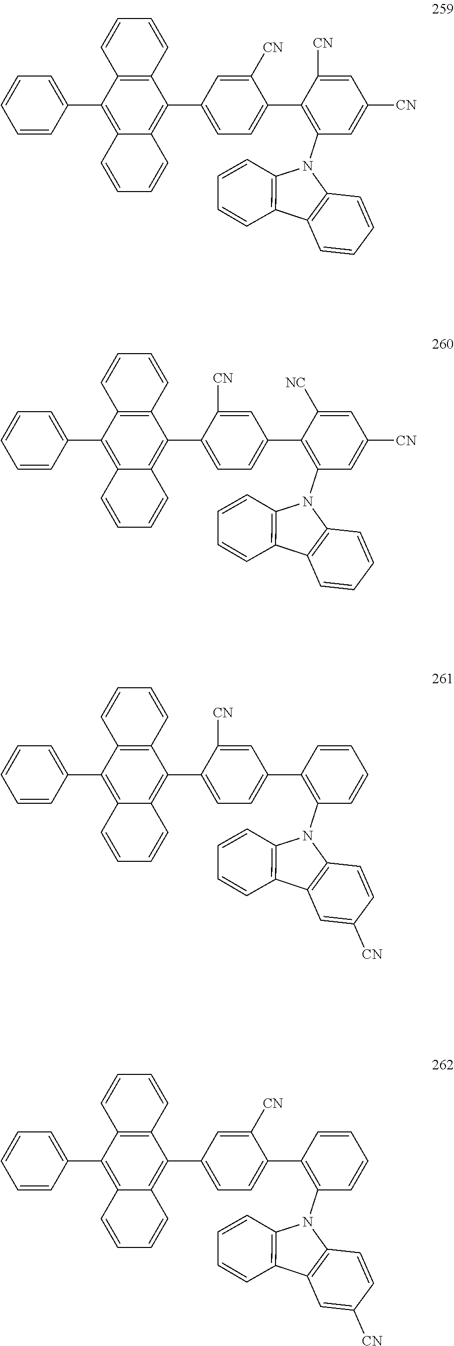 [SMILES: N#Cc1cc(C#N)c(-c2ccc(-c3c4ccccc4c(-c4ccccc4)c4ccccc34)cc2C#N)c(-n2c3ccccc3c3ccccc32)c1 ; N#Cc1cc(C#N)c(-c2ccc(-c3c4ccccc4c(-c4ccccc4)c4ccccc34)c(C#N)c2)c(-n2c3ccccc3c3ccccc32)c1 ; N#Cc1ccc2c(c1)c1ccccc1n2-c1ccccc1-c1ccc(-c2c3ccccc3c(-c3ccccc3)c3ccccc23)c(C#N)c1 ; N#Cc1ccc2c(c1)c1ccccc1n2-c1ccccc1-c1ccc(-c2c3ccccc3c(-c3ccccc3)c3ccccc23)cc1C#N]

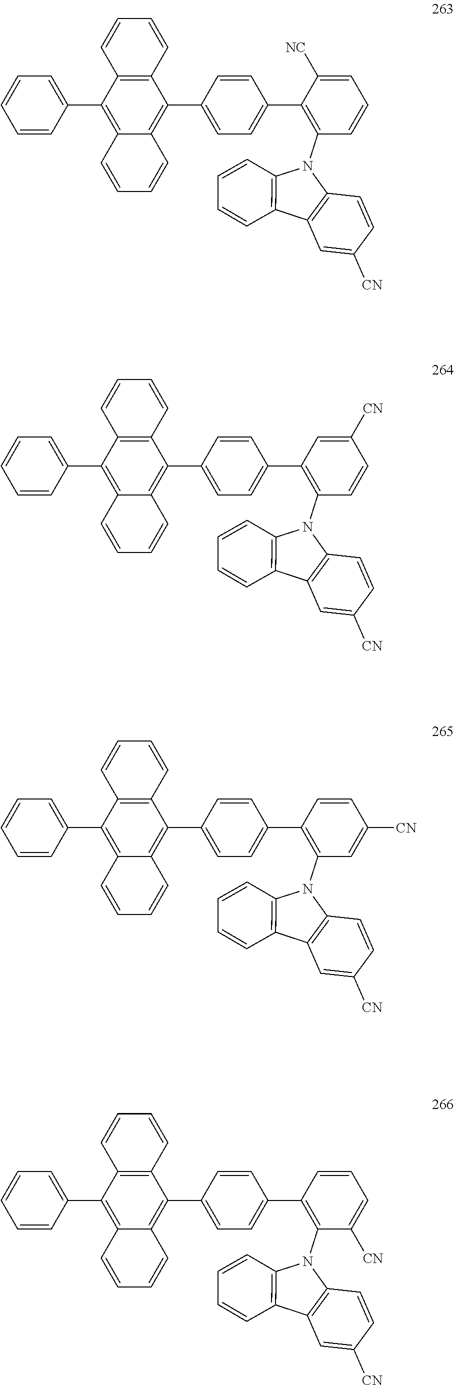 [SMILES: N#Cc1ccc2c(c1)c1ccccc1n2-c1cccc(C#N)c1-c1ccc(-c2c3ccccc3c(-c3ccccc3)c3ccccc23)cc1 ; N#Cc1ccc(-n2c3ccccc3c3cc(C#N)ccc32)c(-c2ccc(-c3c4ccccc4c(-c4ccccc4)c4ccccc34)cc2)c1 ; N#Cc1ccc(-c2ccc(-c3c4ccccc4c(-c4ccccc4)c4ccccc34)cc2)c(-n2c3ccccc3c3cc(C#N)ccc32)c1 ; N#Cc1ccc2c(c1)c1ccccc1n2-c1c(C#N)cccc1-c1ccc(-c2c3ccccc3c(-c3ccccc3)c3ccccc23)cc1]

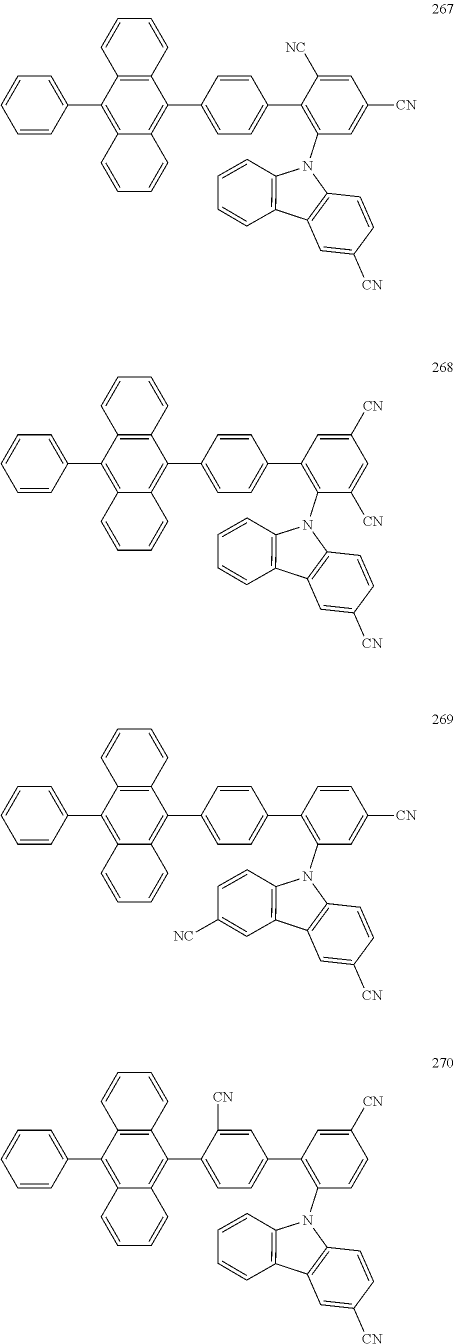 [SMILES: N#Cc1cc(C#N)c(-c2ccc(-c3c4ccccc4c(-c4ccccc4)c4ccccc34)cc2)c(-n2c3ccccc3c3cc(C#N)ccc32)c1 ; N#Cc1cc(C#N)c(-n2c3ccccc3c3cc(C#N)ccc32)c(-c2ccc(-c3c4ccccc4c(-c4ccccc4)c4ccccc34)cc2)c1 ; N#Cc1ccc(-c2ccc(-c3c4ccccc4c(-c4ccccc4)c4ccccc34)cc2)c(-n2c3ccc(C#N)cc3c3cc(C#N)ccc32)c1 ; N#Cc1ccc(-n2c3ccccc3c3cc(C#N)ccc32)c(-c2ccc(-c3c4ccccc4c(-c4ccccc4)c4ccccc34)c(C#N)c2)c1]

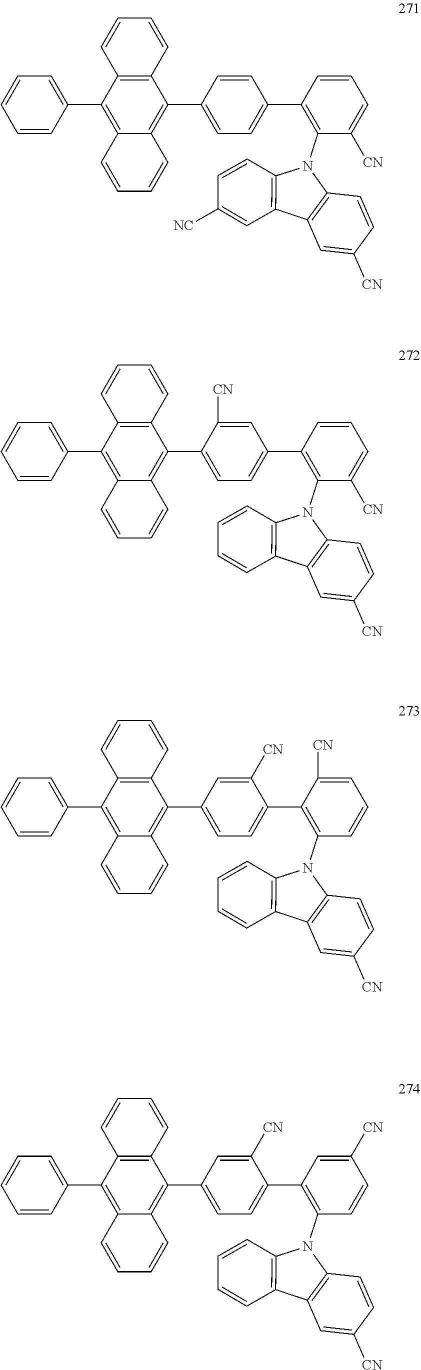 [SMILES: N#Cc1ccc2c(c1)c1cc(C#N)ccc1n2-c1c(C#N)cccc1-c1ccc(-c2c3ccccc3c(-c3ccccc3)c3ccccc23)cc1 ; N#Cc1ccc2c(c1)c1ccccc1n2-c1c(C#N)cccc1-c1ccc(-c2c3ccccc3c(-c3ccccc3)c3ccccc23)c(C#N)c1 ; N#Cc1ccc2c(c1)c1ccccc1n2-c1cccc(C#N)c1-c1ccc(-c2c3ccccc3c(-c3ccccc3)c3ccccc23)cc1C#N ; N#Cc1ccc(-n2c3ccccc3c3cc(C#N)ccc32)c(-c2ccc(-c3c4ccccc4c(-c4ccccc4)c4ccccc34)cc2C#N)c1]

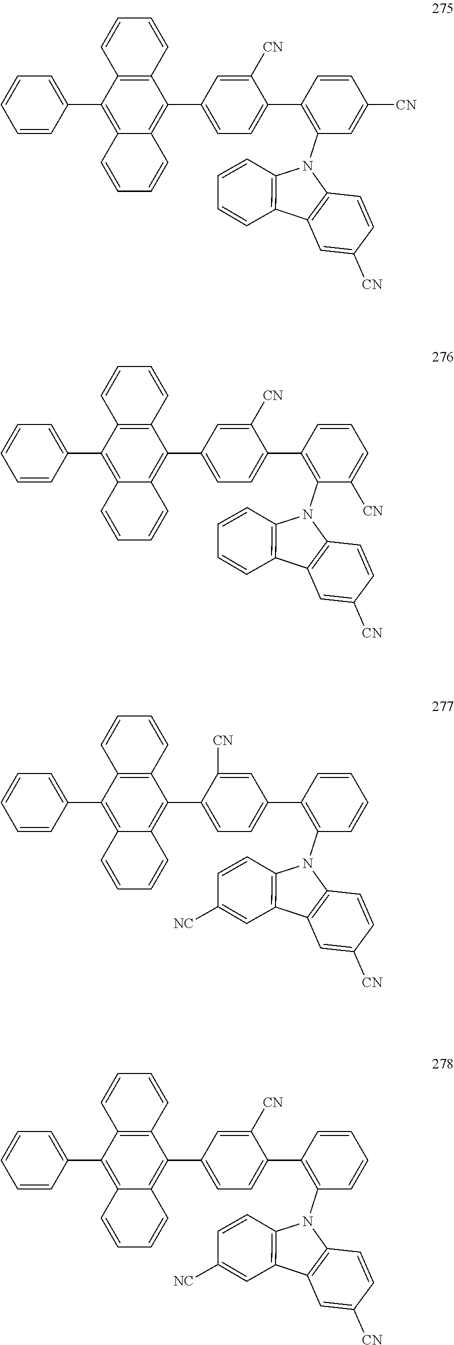 [SMILES: N#Cc1ccc(-c2ccc(-c3c4ccccc4c(-c4ccccc4)c4ccccc34)cc2C#N)c(-n2c3ccccc3c3cc(C#N)ccc32)c1 ; N#Cc1ccc2c(c1)c1ccccc1n2-c1c(C#N)cccc1-c1ccc(-c2c3ccccc3c(-c3ccccc3)c3ccccc23)cc1C#N ; N#Cc1ccc2c(c1)c1cc(C#N)ccc1n2-c1ccccc1-c1ccc(-c2c3ccccc3c(-c3ccccc3)c3ccccc23)c(C#N)c1 ; N#Cc1ccc2c(c1)c1cc(C#N)ccc1n2-c1ccccc1-c1ccc(-c2c3ccccc3c(-c3ccccc3)c3ccccc23)cc1C#N]

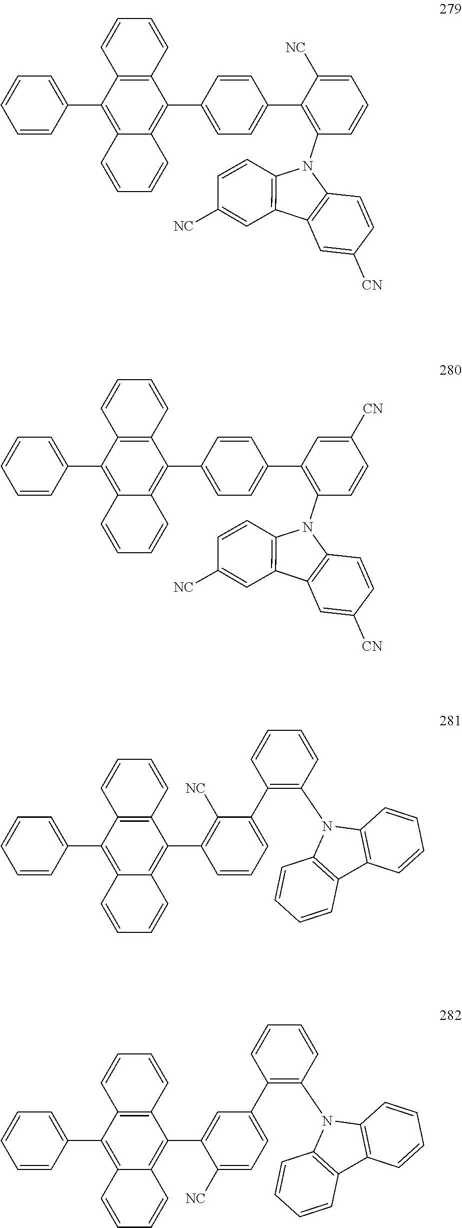 [SMILES: N#Cc1ccc2c(c1)c1cc(C#N)ccc1n2-c1cccc(C#N)c1-c1ccc(-c2c3ccccc3c(-c3ccccc3)c3ccccc23)cc1 ; N#Cc1ccc(-n2c3ccc(C#N)cc3c3cc(C#N)ccc32)c(-c2ccc(-c3c4ccccc4c(-c4ccccc4)c4ccccc34)cc2)c1 ; N#Cc1c(-c2ccccc2-n2c3ccccc3c3ccccc32)cccc1-c1c2ccccc2c(-c2ccccc2)c2ccccc12 ; N#Cc1ccc(-c2ccccc2-n2c3ccccc3c3ccccc32)cc1-c1c2ccccc2c(-c2ccccc2)c2ccccc12]

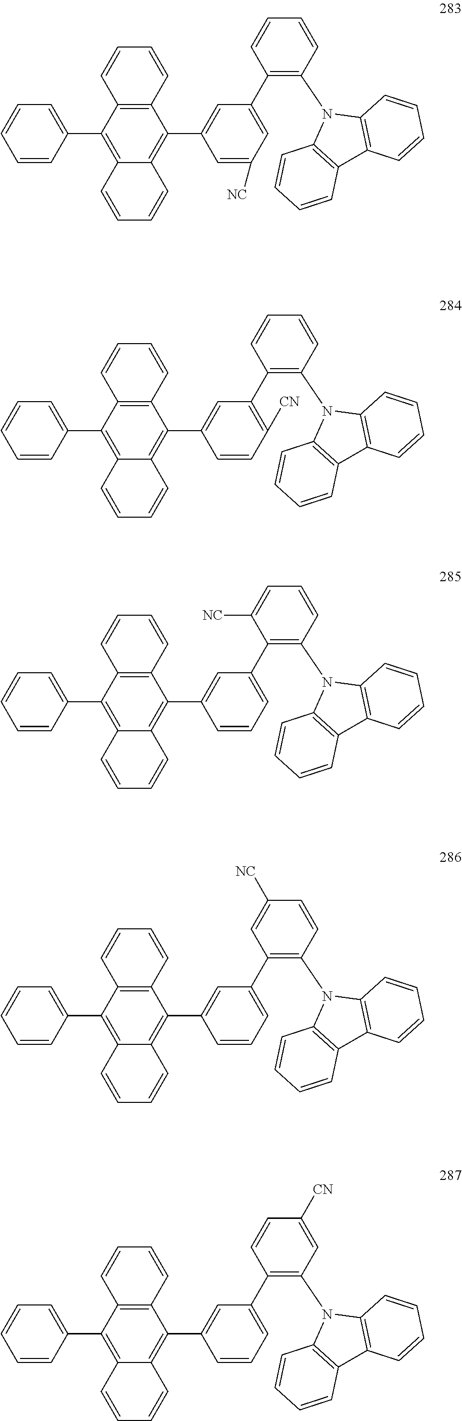 [SMILES: N#Cc1cc(-c2ccccc2-n2c3ccccc3c3ccccc32)cc(-c2c3ccccc3c(-c3ccccc3)c3ccccc23)c1 ; N#Cc1ccc(-c2c3ccccc3c(-c3ccccc3)c3ccccc23)cc1-c1ccccc1-n1c2ccccc2c2ccccc21 ; N#Cc1cccc(-n2c3ccccc3c3ccccc32)c1-c1cccc(-c2c3ccccc3c(-c3ccccc3)c3ccccc23)c1 ; N#Cc1ccc(-n2c3ccccc3c3ccccc32)c(-c2cccc(-c3c4ccccc4c(-c4ccccc4)c4ccccc34)c2)c1 ; N#Cc1ccc(-c2cccc(-c3c4ccccc4c(-c4ccccc4)c4ccccc34)c2)c(-n2c3ccccc3c3ccccc32)c1]

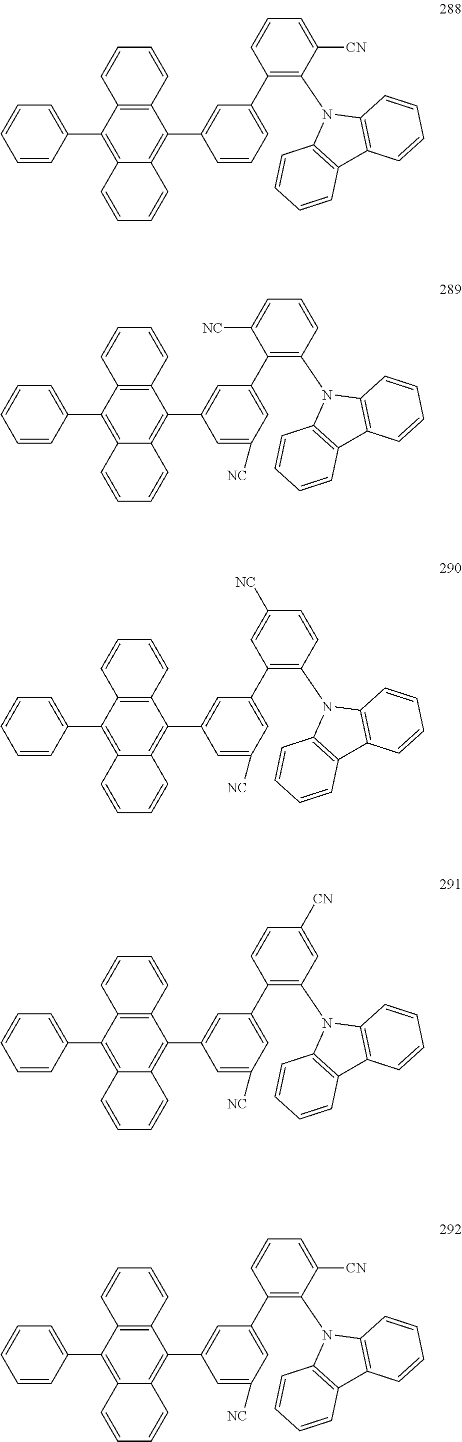 [SMILES: N#Cc1cccc(-c2cccc(-c3c4ccccc4c(-c4ccccc4)c4ccccc34)c2)c1-n1c2ccccc2c2ccccc21 ; N#Cc1cc(-c2c(C#N)cccc2-n2c3ccccc3c3ccccc32)cc(-c2c3ccccc3c(-c3ccccc3)c3ccccc23)c1 ; N#Cc1cc(-c2cc(C#N)ccc2-n2c3ccccc3c3ccccc32)cc(-c2c3ccccc3c(-c3ccccc3)c3ccccc23)c1 ; N#Cc1cc(-c2ccc(C#N)cc2-n2c3ccccc3c3ccccc32)cc(-c2c3ccccc3c(-c3ccccc3)c3ccccc23)c1 ; N#Cc1cc(-c2cccc(C#N)c2-n2c3ccccc3c3ccccc32)cc(-c2c3ccccc3c(-c3ccccc3)c3ccccc23)c1]

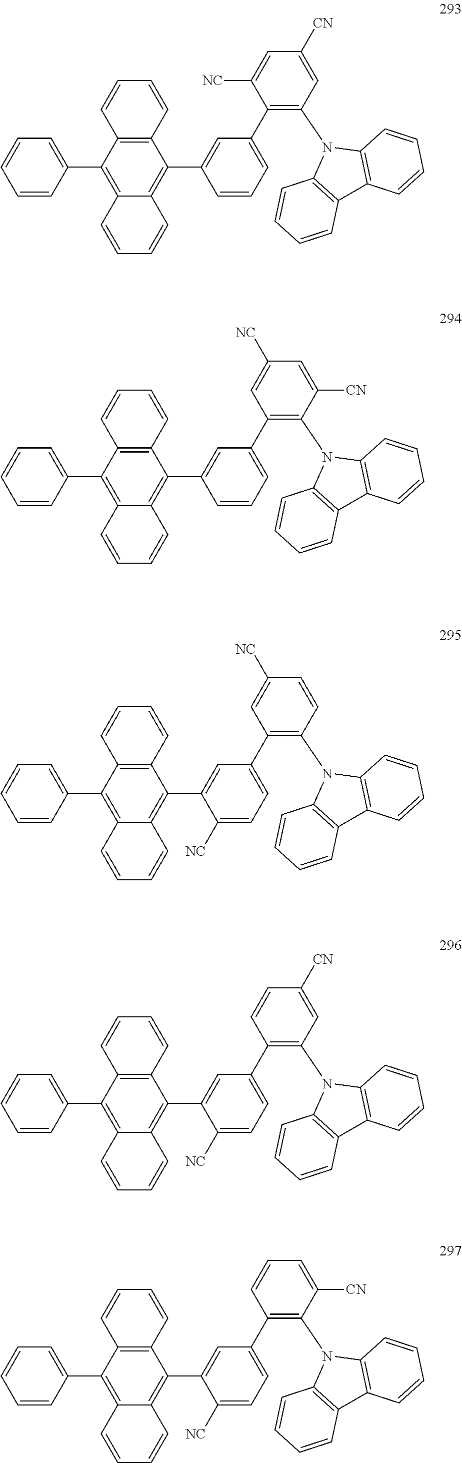 [SMILES: N#Cc1cc(C#N)c(-c2cccc(-c3c4ccccc4c(-c4ccccc4)c4ccccc34)c2)c(-n2c3ccccc3c3ccccc32)c1 ; N#Cc1cc(C#N)c(-n2c3ccccc3c3ccccc32)c(-c2cccc(-c3c4ccccc4c(-c4ccccc4)c4ccccc34)c2)c1 ; N#Cc1ccc(-n2c3ccccc3c3ccccc32)c(-c2ccc(C#N)c(-c3c4ccccc4c(-c4ccccc4)c4ccccc34)c2)c1 ; N#Cc1ccc(-c2ccc(C#N)c(-c3c4ccccc4c(-c4ccccc4)c4ccccc34)c2)c(-n2c3ccccc3c3ccccc32)c1 ; N#Cc1ccc(-c2cccc(C#N)c2-n2c3ccccc3c3ccccc32)cc1-c1c2ccccc2c(-c2ccccc2)c2ccccc12]

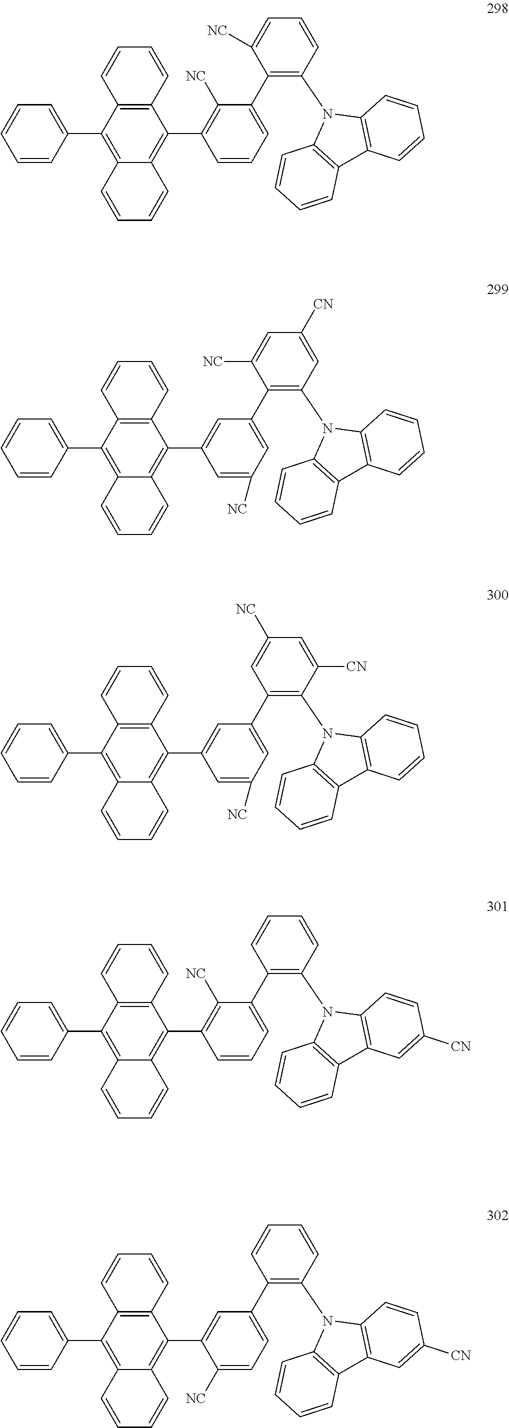 [SMILES: N#Cc1cccc(-n2c3ccccc3c3ccccc32)c1-c1cccc(-c2c3ccccc3c(-c3ccccc3)c3ccccc23)c1C#N ; N#Cc1cc(-c2c(C#N)cc(C#N)cc2-n2c3ccccc3c3ccccc32)cc(-c2c3ccccc3c(-c3ccccc3)c3ccccc23)c1 ; N#Cc1cc(-c2cc(C#N)cc(C#N)c2-n2c3ccccc3c3ccccc32)cc(-c2c3ccccc3c(-c3ccccc3)c3ccccc23)c1 ; N#Cc1ccc2c(c1)c1ccccc1n2-c1ccccc1-c1cccc(-c2c3ccccc3c(-c3ccccc3)c3ccccc23)c1C#N ; N#Cc1ccc2c(c1)c1ccccc1n2-c1ccccc1-c1ccc(C#N)c(-c2c3ccccc3c(-c3ccccc3)c3ccccc23)c1]

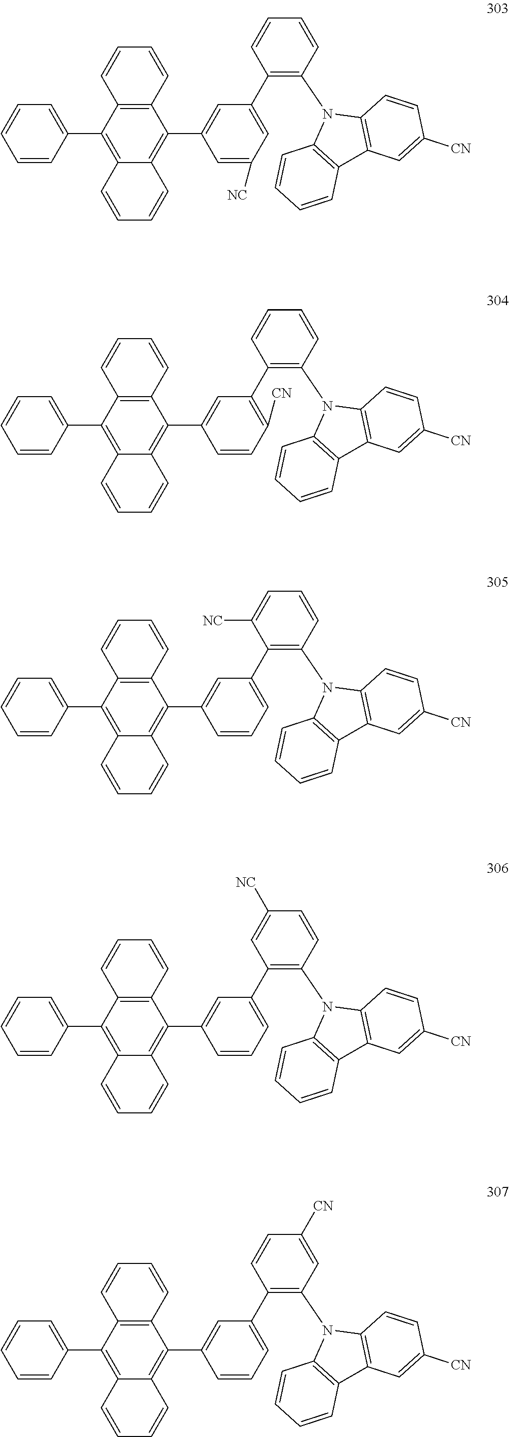 [SMILES: N#Cc1cc(-c2ccccc2-n2c3ccccc3c3cc(C#N)ccc32)cc(-c2c3ccccc3c(-c3ccccc3)c3ccccc23)c1 ; N#Cc1ccc2c(c1)c1ccccc1n2-c1ccccc1-c1cc(-c2c3ccccc3c(-c3ccccc3)c3ccccc23)ccc1C#N ; N#Cc1ccc2c(c1)c1ccccc1n2-c1cccc(C#N)c1-c1cccc(-c2c3ccccc3c(-c3ccccc3)c3ccccc23)c1 ; N#Cc1ccc(-n2c3ccccc3c3cc(C#N)ccc32)c(-c2cccc(-c3c4ccccc4c(-c4ccccc4)c4ccccc34)c2)c1 ; N#Cc1ccc(-c2cccc(-c3c4ccccc4c(-c4ccccc4)c4ccccc34)c2)c(-n2c3ccccc3c3cc(C#N)ccc32)c1]

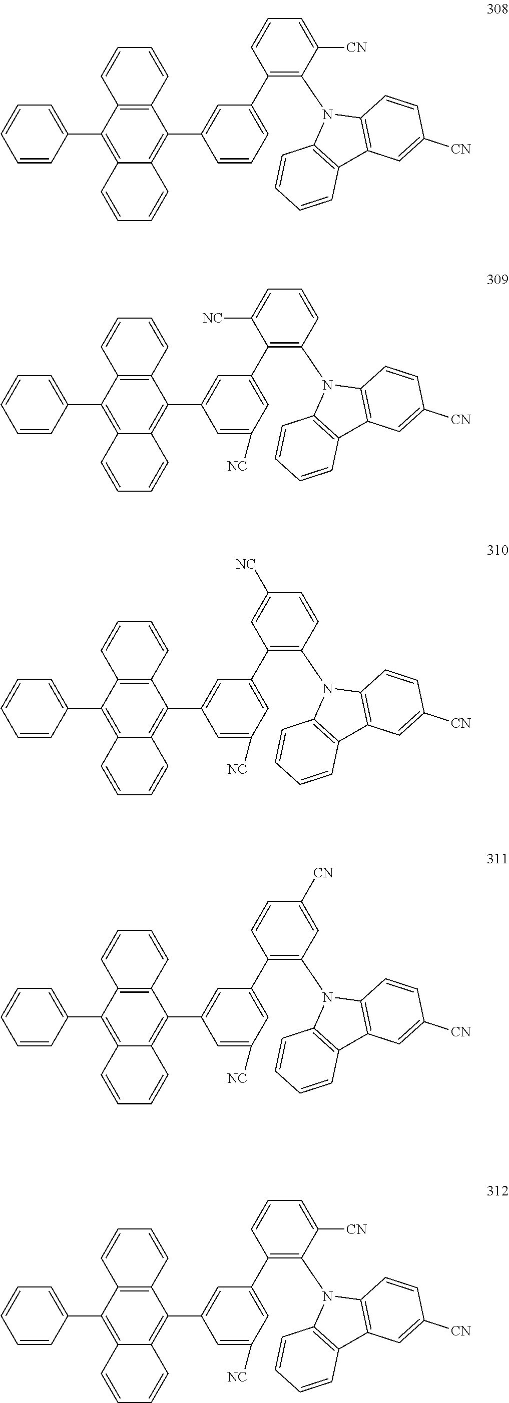 [SMILES: N#Cc1ccc2c(c1)c1ccccc1n2-c1c(C#N)cccc1-c1cccc(-c2c3ccccc3c(-c3ccccc3)c3ccccc23)c1 ; N#Cc1cc(-c2c(C#N)cccc2-n2c3ccccc3c3cc(C#N)ccc32)cc(-c2c3ccccc3c(-c3ccccc3)c3ccccc23)c1 ; N#Cc1cc(-c2cc(C#N)ccc2-n2c3ccccc3c3cc(C#N)ccc32)cc(-c2c3ccccc3c(-c3ccccc3)c3ccccc23)c1 ; N#Cc1cc(-c2ccc(C#N)cc2-n2c3ccccc3c3cc(C#N)ccc32)cc(-c2c3ccccc3c(-c3ccccc3)c3ccccc23)c1 ; N#Cc1cc(-c2cccc(C#N)c2-n2c3ccccc3c3cc(C#N)ccc32)cc(-c2c3ccccc3c(-c3ccccc3)c3ccccc23)c1]

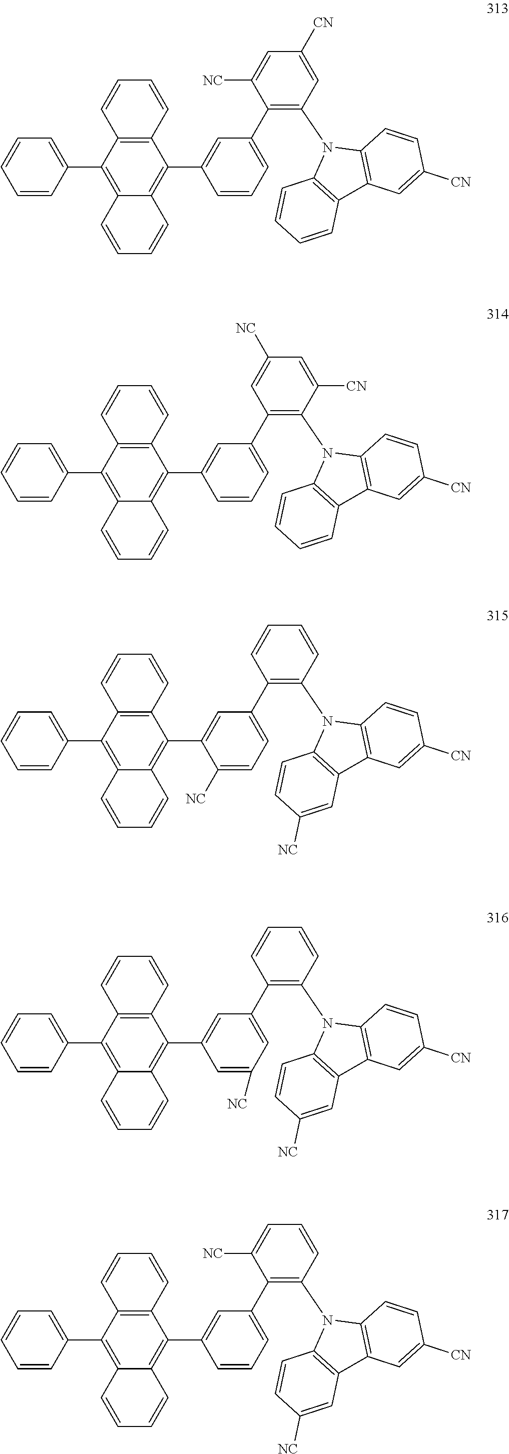 [SMILES: N#Cc1cc(C#N)c(-c2cccc(-c3c4ccccc4c(-c4ccccc4)c4ccccc34)c2)c(-n2c3ccccc3c3cc(C#N)ccc32)c1 ; N#Cc1cc(C#N)c(-n2c3ccccc3c3cc(C#N)ccc32)c(-c2cccc(-c3c4ccccc4c(-c4ccccc4)c4ccccc34)c2)c1 ; N#Cc1ccc2c(c1)c1cc(C#N)ccc1n2-c1ccccc1-c1ccc(C#N)c(-c2c3ccccc3c(-c3ccccc3)c3ccccc23)c1 ; N#Cc1cc(-c2ccccc2-n2c3ccc(C#N)cc3c3cc(C#N)ccc32)cc(-c2c3ccccc3c(-c3ccccc3)c3ccccc23)c1 ; N#Cc1ccc2c(c1)c1cc(C#N)ccc1n2-c1cccc(C#N)c1-c1cccc(-c2c3ccccc3c(-c3ccccc3)c3ccccc23)c1]

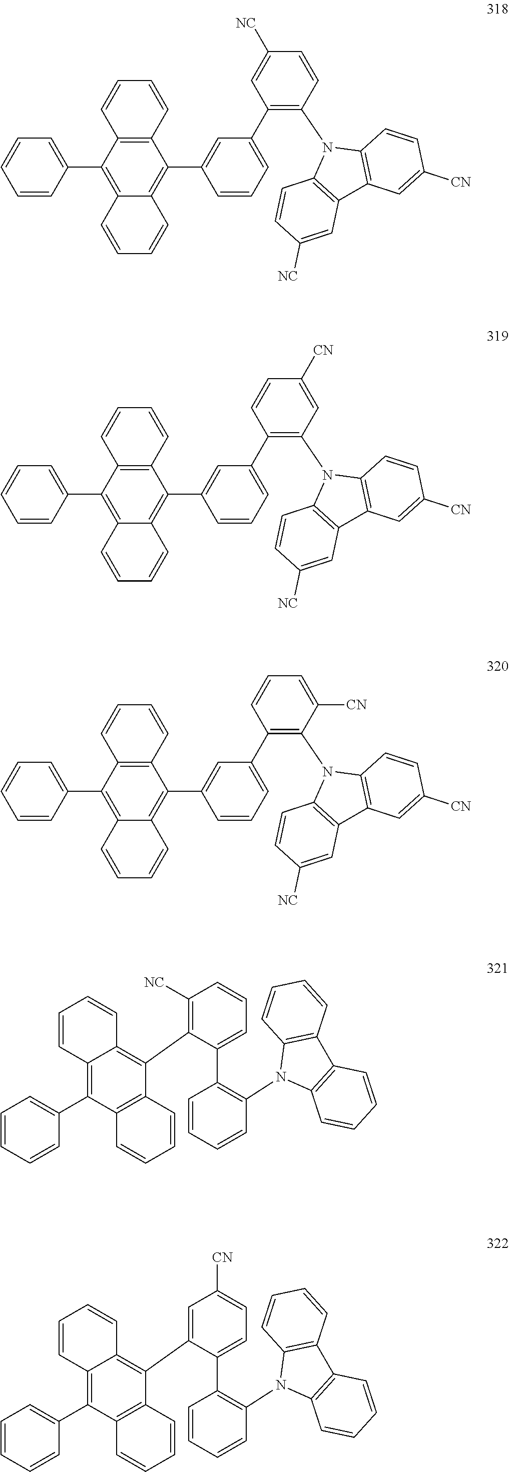 [SMILES: N#Cc1ccc(-n2c3ccc(C#N)cc3c3cc(C#N)ccc32)c(-c2cccc(-c3c4ccccc4c(-c4ccccc4)c4ccccc34)c2)c1 ; N#Cc1ccc(-c2cccc(-c3c4ccccc4c(-c4ccccc4)c4ccccc34)c2)c(-n2c3ccc(C#N)cc3c3cc(C#N)ccc32)c1 ; N#Cc1ccc2c(c1)c1cc(C#N)ccc1n2-c1c(C#N)cccc1-c1cccc(-c2c3ccccc3c(-c3ccccc3)c3ccccc23)c1 ; N#Cc1cccc(-c2ccccc2-n2c3ccccc3c3ccccc32)c1-c1c2ccccc2c(-c2ccccc2)c2ccccc12 ; N#Cc1ccc(-c2ccccc2-n2c3ccccc3c3ccccc32)c(-c2c3ccccc3c(-c3ccccc3)c3ccccc23)c1]

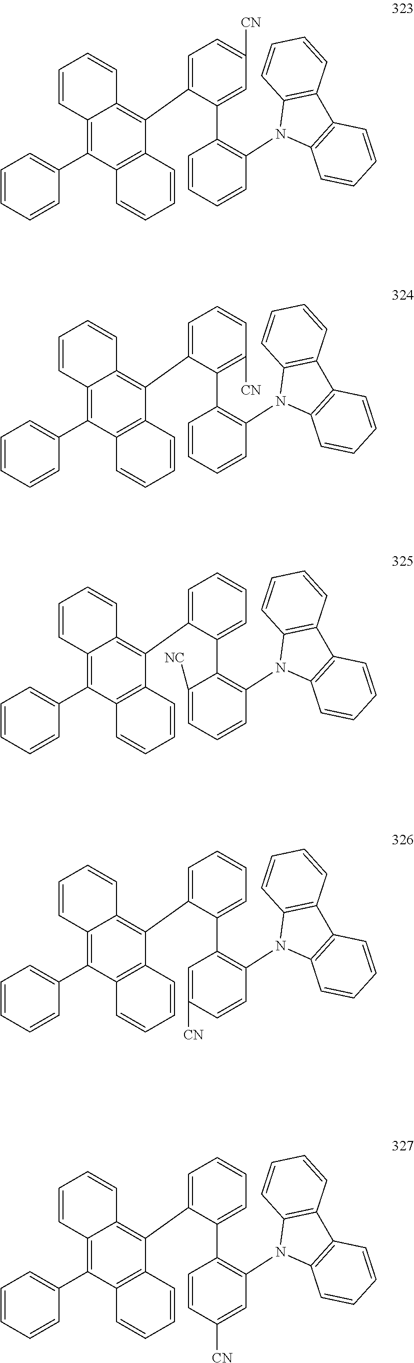 [SMILES: N#Cc1ccc(-c2c3ccccc3c(-c3ccccc3)c3ccccc23)c(-c2ccccc2-n2c3ccccc3c3ccccc32)c1 ; N#Cc1cccc(-c2c3ccccc3c(-c3ccccc3)c3ccccc23)c1-c1ccccc1-n1c2ccccc2c2ccccc21 ; N#Cc1cccc(-n2c3ccccc3c3ccccc32)c1-c1ccccc1-c1c2ccccc2c(-c2ccccc2)c2ccccc12 ; N#Cc1ccc(-n2c3ccccc3c3ccccc32)c(-c2ccccc2-c2c3ccccc3c(-c3ccccc3)c3ccccc23)c1 ; N#Cc1ccc(-c2ccccc2-c2c3ccccc3c(-c3ccccc3)c3ccccc23)c(-n2c3ccccc3c3ccccc32)c1]

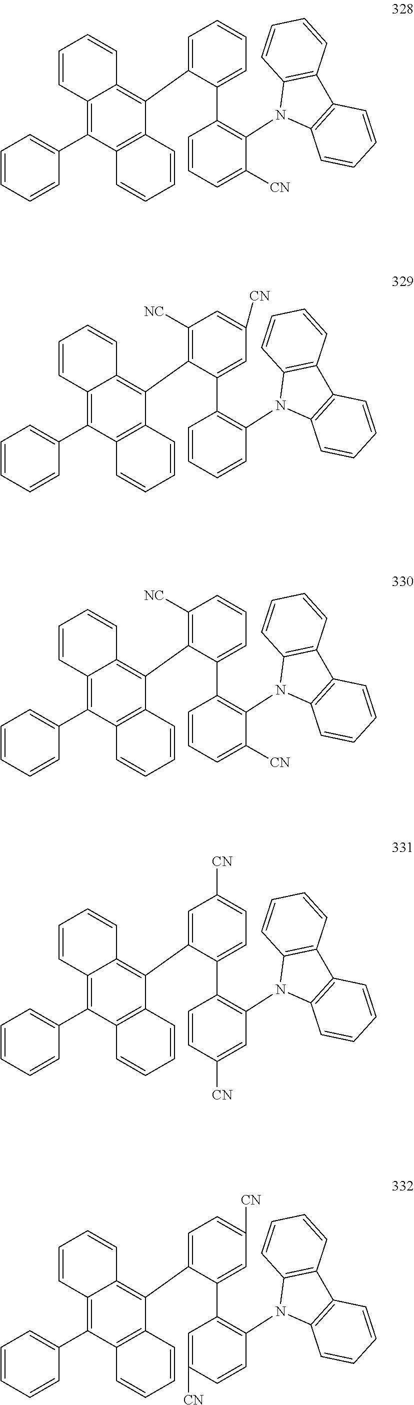 [SMILES: N#Cc1cccc(-c2ccccc2-c2c3ccccc3c(-c3ccccc3)c3ccccc23)c1-n1c2ccccc2c2ccccc21 ; N#Cc1cc(C#N)c(-c2c3ccccc3c(-c3ccccc3)c3ccccc23)c(-c2ccccc2-n2c3ccccc3c3ccccc32)c1 ; N#Cc1cccc(-c2cccc(C#N)c2-n2c3ccccc3c3ccccc32)c1-c1c2ccccc2c(-c2ccccc2)c2ccccc12 ; N#Cc1ccc(-c2ccc(C#N)cc2-n2c3ccccc3c3ccccc32)c(-c2c3ccccc3c(-c3ccccc3)c3ccccc23)c1 ; N#Cc1ccc(-c2c3ccccc3c(-c3ccccc3)c3ccccc23)c(-c2cc(C#N)ccc2-n2c3ccccc3c3ccccc32)c1]

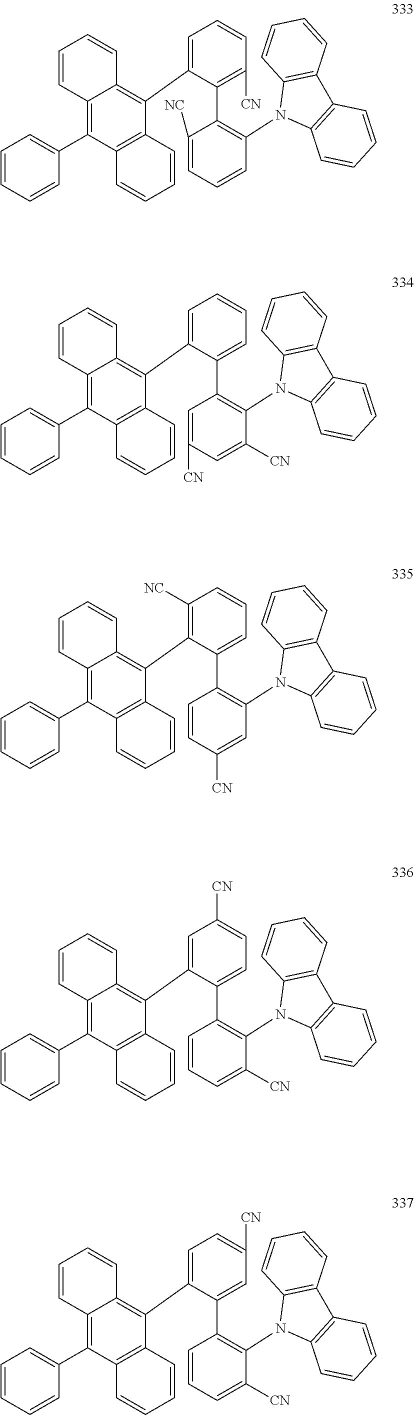 [SMILES: N#Cc1cccc(-c2c3ccccc3c(-c3ccccc3)c3ccccc23)c1-c1c(C#N)cccc1-n1c2ccccc2c2ccccc21 ; N#Cc1cc(C#N)c(-n2c3ccccc3c3ccccc32)c(-c2ccccc2-c2c3ccccc3c(-c3ccccc3)c3ccccc23)c1 ; N#Cc1ccc(-c2cccc(C#N)c2-c2c3ccccc3c(-c3ccccc3)c3ccccc23)c(-n2c3ccccc3c3ccccc32)c1 ; N#Cc1ccc(-c2cccc(C#N)c2-n2c3ccccc3c3ccccc32)c(-c2c3ccccc3c(-c3ccccc3)c3ccccc23)c1 ; N#Cc1ccc(-c2c3ccccc3c(-c3ccccc3)c3ccccc23)c(-c2cccc(C#N)c2-n2c3ccccc3c3ccccc32)c1]

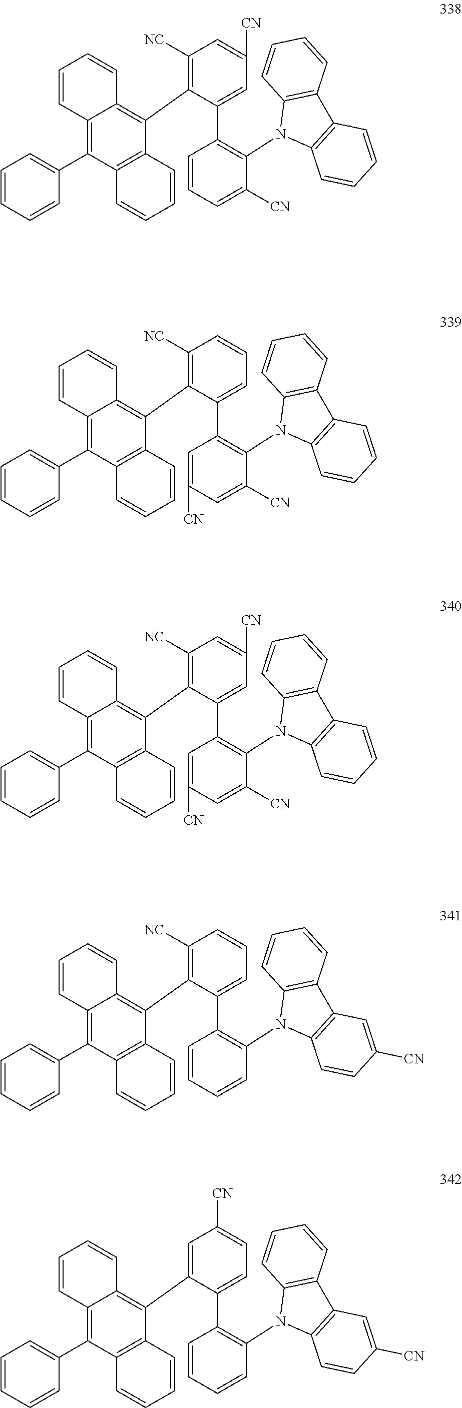 [SMILES: N#Cc1cc(C#N)c(-c2c3ccccc3c(-c3ccccc3)c3ccccc23)c(-c2cccc(C#N)c2-n2c3ccccc3c3ccccc32)c1 ; N#Cc1cc(C#N)c(-n2c3ccccc3c3ccccc32)c(-c2cccc(C#N)c2-c2c3ccccc3c(-c3ccccc3)c3ccccc23)c1 ; N#Cc1cc(C#N)c(-c2c3ccccc3c(-c3ccccc3)c3ccccc23)c(-c2cc(C#N)cc(C#N)c2-n2c3ccccc3c3ccccc32)c1 ; N#Cc1ccc2c(c1)c1ccccc1n2-c1ccccc1-c1cccc(C#N)c1-c1c2ccccc2c(-c2ccccc2)c2ccccc12 ; N#Cc1ccc(-c2ccccc2-n2c3ccccc3c3cc(C#N)ccc32)c(-c2c3ccccc3c(-c3ccccc3)c3ccccc23)c1]

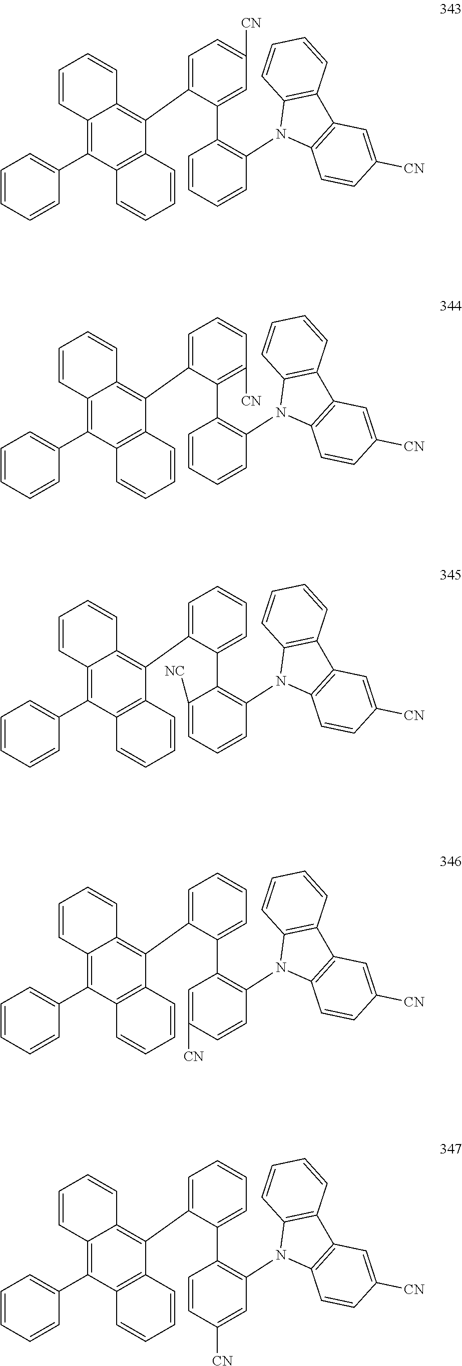 [SMILES: N#Cc1ccc(-c2c3ccccc3c(-c3ccccc3)c3ccccc23)c(-c2ccccc2-n2c3ccccc3c3cc(C#N)ccc32)c1 ; N#Cc1ccc2c(c1)c1ccccc1n2-c1ccccc1-c1c(C#N)cccc1-c1c2ccccc2c(-c2ccccc2)c2ccccc12 ; N#Cc1ccc2c(c1)c1ccccc1n2-c1cccc(C#N)c1-c1ccccc1-c1c2ccccc2c(-c2ccccc2)c2ccccc12 ; N#Cc1ccc(-n2c3ccccc3c3cc(C#N)ccc32)c(-c2ccccc2-c2c3ccccc3c(-c3ccccc3)c3ccccc23)c1 ; N#Cc1ccc(-c2ccccc2-c2c3ccccc3c(-c3ccccc3)c3ccccc23)c(-n2c3ccccc3c3cc(C#N)ccc32)c1]

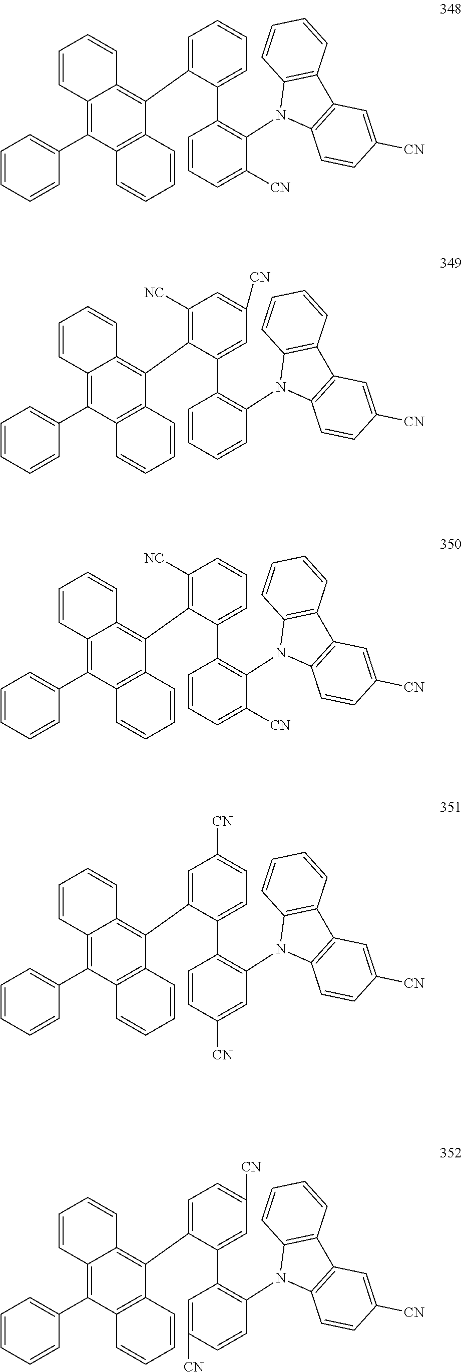 [SMILES: N#Cc1ccc2c(c1)c1ccccc1n2-c1c(C#N)cccc1-c1ccccc1-c1c2ccccc2c(-c2ccccc2)c2ccccc12 ; N#Cc1cc(C#N)c(-c2c3ccccc3c(-c3ccccc3)c3ccccc23)c(-c2ccccc2-n2c3ccccc3c3cc(C#N)ccc32)c1 ; N#Cc1ccc2c(c1)c1ccccc1n2-c1c(C#N)cccc1-c1cccc(C#N)c1-c1c2ccccc2c(-c2ccccc2)c2ccccc12 ; N#Cc1ccc(-c2ccc(C#N)cc2-n2c3ccccc3c3cc(C#N)ccc32)c(-c2c3ccccc3c(-c3ccccc3)c3ccccc23)c1 ; N#Cc1ccc(-c2c3ccccc3c(-c3ccccc3)c3ccccc23)c(-c2cc(C#N)ccc2-n2c3ccccc3c3cc(C#N)ccc32)c1]

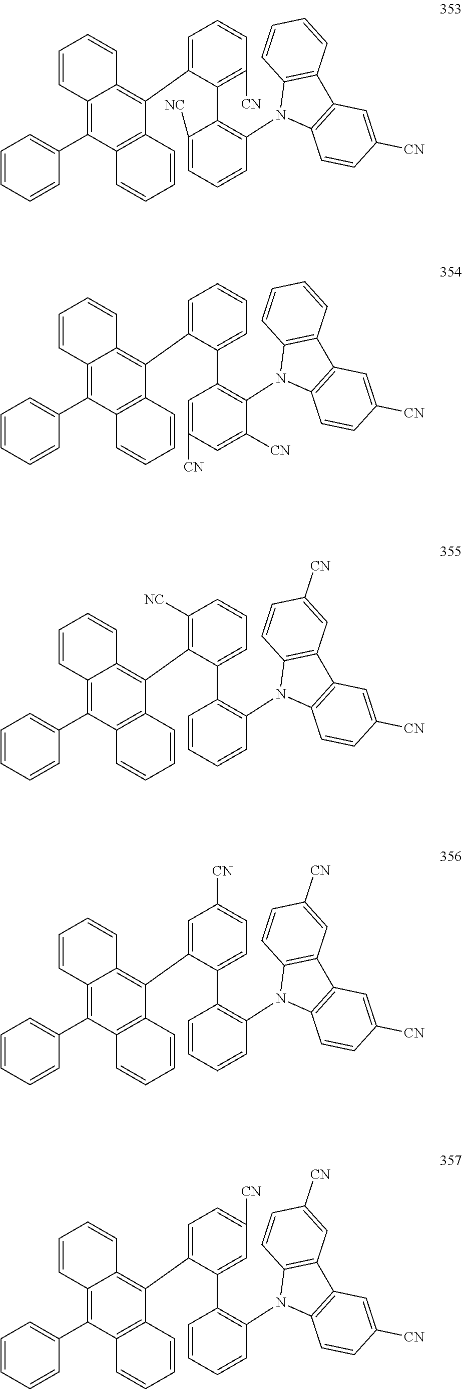 [SMILES: N#Cc1ccc2c(c1)c1ccccc1n2-c1cccc(C#N)c1-c1c(C#N)cccc1-c1c2ccccc2c(-c2ccccc2)c2ccccc12 ; N#Cc1cc(C#N)c(-n2c3ccccc3c3cc(C#N)ccc32)c(-c2ccccc2-c2c3ccccc3c(-c3ccccc3)c3ccccc23)c1 ; N#Cc1ccc2c(c1)c1cc(C#N)ccc1n2-c1ccccc1-c1cccc(C#N)c1-c1c2ccccc2c(-c2ccccc2)c2ccccc12 ; N#Cc1ccc(-c2ccccc2-n2c3ccc(C#N)cc3c3cc(C#N)ccc32)c(-c2c3ccccc3c(-c3ccccc3)c3ccccc23)c1 ; N#Cc1ccc(-c2c3ccccc3c(-c3ccccc3)c3ccccc23)c(-c2ccccc2-n2c3ccc(C#N)cc3c3cc(C#N)ccc32)c1]

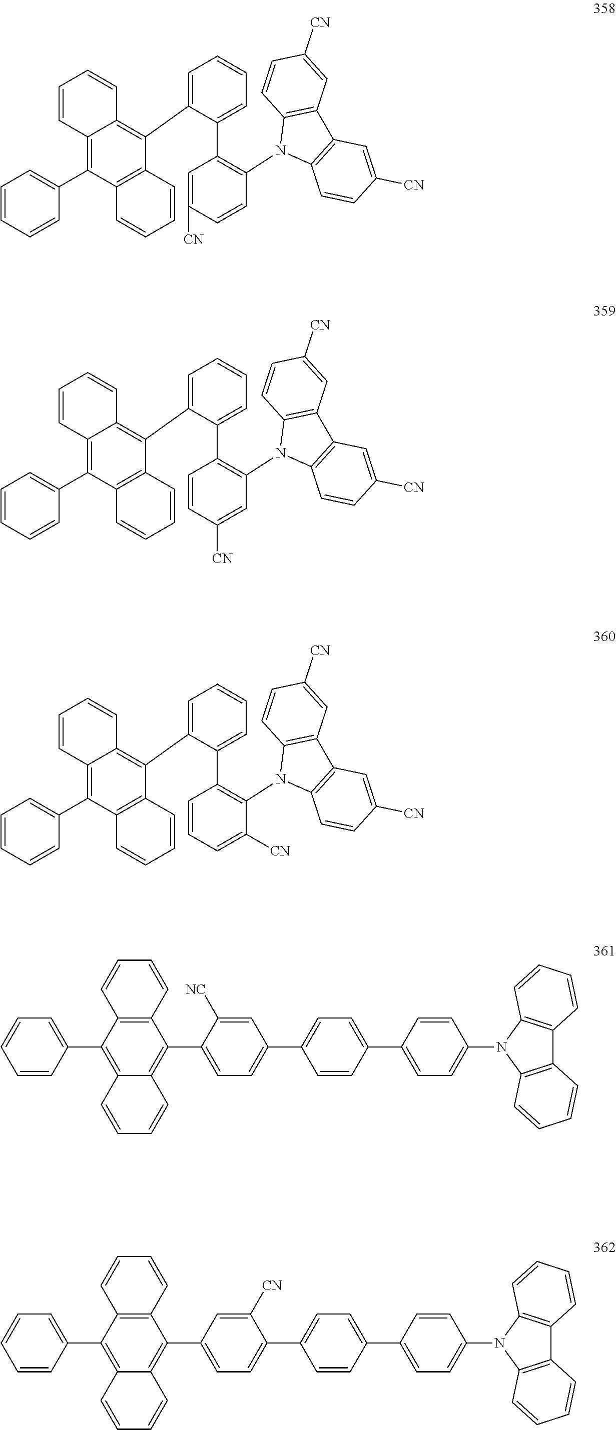 [SMILES: N#Cc1ccc(-n2c3ccc(C#N)cc3c3cc(C#N)ccc32)c(-c2ccccc2-c2c3ccccc3c(-c3ccccc3)c3ccccc23)c1 ; N#Cc1ccc(-c2ccccc2-c2c3ccccc3c(-c3ccccc3)c3ccccc23)c(-n2c3ccc(C#N)cc3c3cc(C#N)ccc32)c1 ; N#Cc1ccc2c(c1)c1cc(C#N)ccc1n2-c1c(C#N)cccc1-c1ccccc1-c1c2ccccc2c(-c2ccccc2)c2ccccc12 ; N#Cc1cc(-c2ccc(-c3ccc(-n4c5ccccc5c5ccccc54)cc3)cc2)ccc1-c1c2ccccc2c(-c2ccccc2)c2ccccc12 ; N#Cc1cc(-c2c3ccccc3c(-c3ccccc3)c3ccccc23)ccc1-c1ccc(-c2ccc(-n3c4ccccc4c4ccccc43)cc2)cc1]

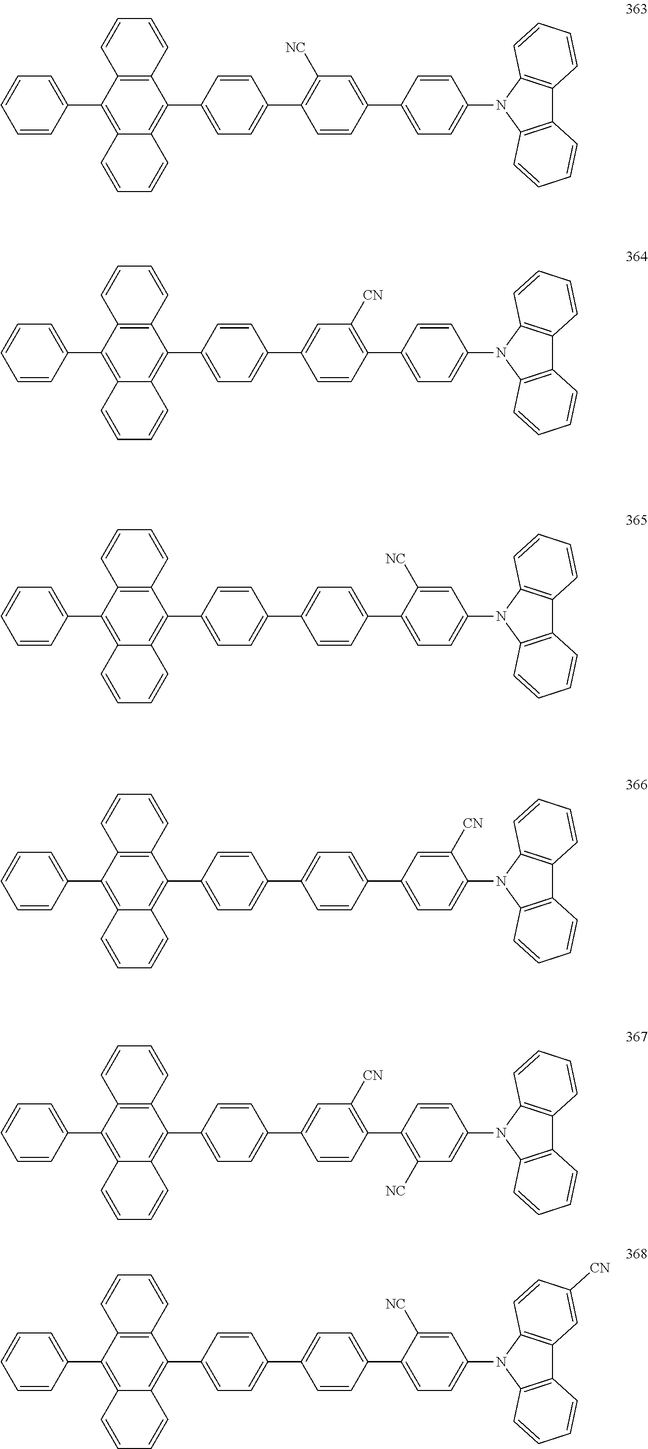

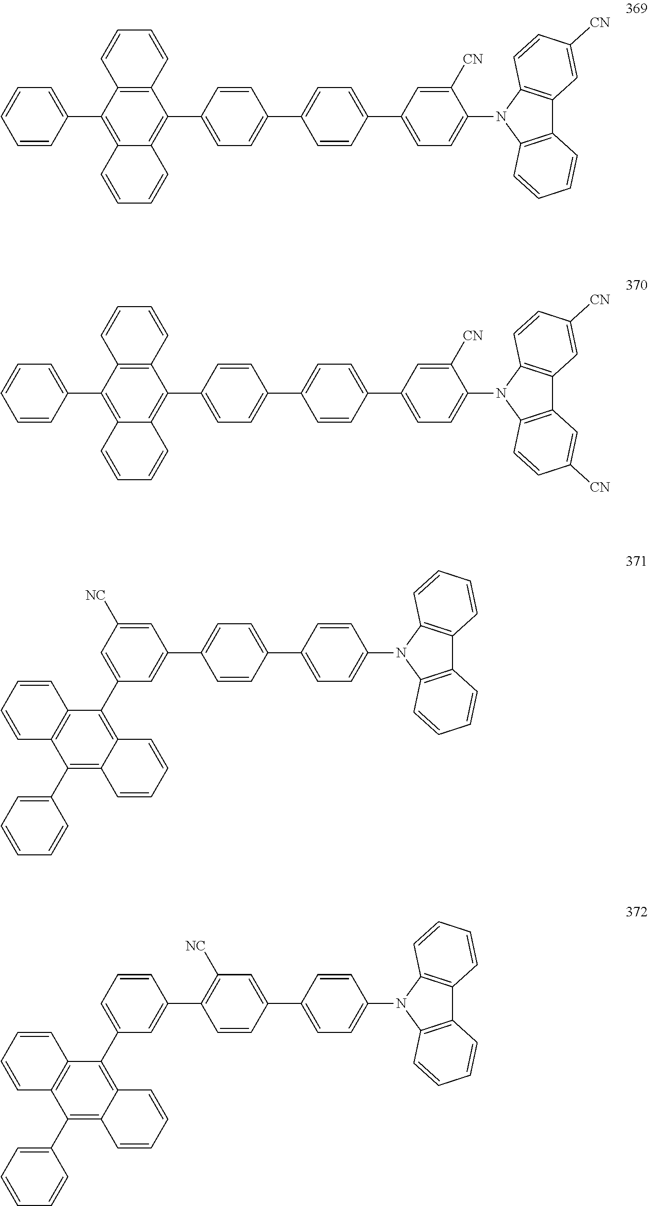 [SMILES: N#Cc1ccc2c(c1)c1ccccc1n2-c1ccc(-c2ccc(-c3ccc(-c4c5ccccc5c(-c5ccccc5)c5ccccc45)cc3)cc2)cc1C#N ; N#Cc1ccc2c(c1)c1cc(C#N)ccc1n2-c1ccc(-c2ccc(-c3ccc(-c4c5ccccc5c(-c5ccccc5)c5ccccc45)cc3)cc2)cc1C#N ; N#Cc1cc(-c2ccc(-c3ccc(-n4c5ccccc5c5ccccc54)cc3)cc2)cc(-c2c3ccccc3c(-c3ccccc3)c3ccccc23)c1 ; N#Cc1cc(-c2ccc(-n3c4ccccc4c4ccccc43)cc2)ccc1-c1cccc(-c2c3ccccc3c(-c3ccccc3)c3ccccc23)c1]

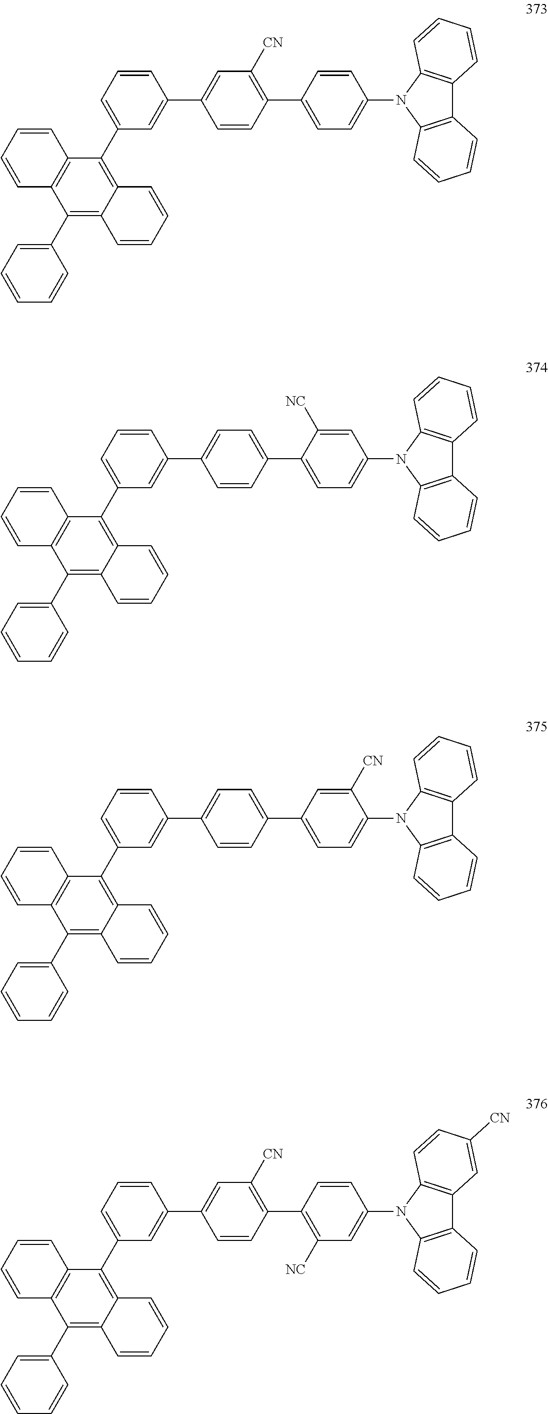 [SMILES: N#Cc1cc(-c2cccc(-c3c4ccccc4c(-c4ccccc4)c4ccccc34)c2)ccc1-c1ccc(-n2c3ccccc3c3ccccc32)cc1 ; N#Cc1cc(-n2c3ccccc3c3ccccc32)ccc1-c1ccc(-c2cccc(-c3c4ccccc4c(-c4ccccc4)c4ccccc34)c2)cc1 ; N#Cc1cc(-c2ccc(-c3cccc(-c4c5ccccc5c(-c5ccccc5)c5ccccc45)c3)cc2)ccc1-n1c2ccccc2c2ccccc21 ; N#Cc1ccc2c(c1)c1ccccc1n2-c1ccc(-c2ccc(-c3cccc(-c4c5ccccc5c(-c5ccccc5)c5ccccc45)c3)cc2C#N)c(C#N)c1]

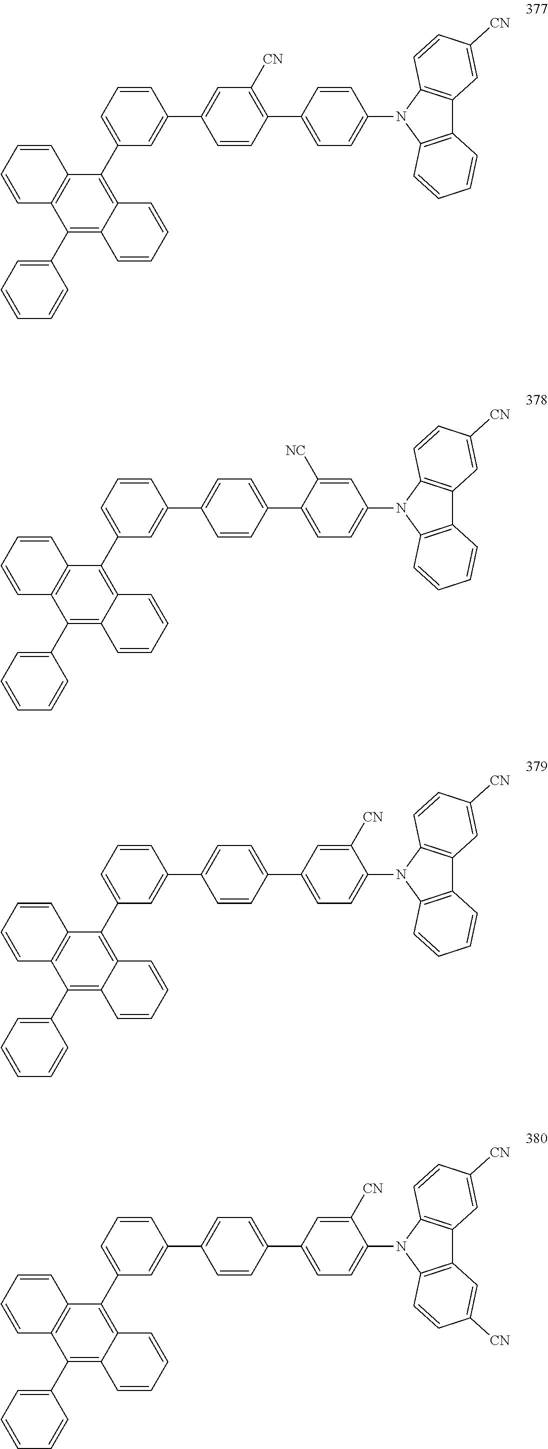 [SMILES: N#Cc1ccc2c(c1)c1ccccc1n2-c1ccc(-c2ccc(-c3cccc(-c4c5ccccc5c(-c5ccccc5)c5ccccc45)c3)cc2C#N)cc1 ; N#Cc1ccc2c(c1)c1ccccc1n2-c1ccc(-c2ccc(-c3cccc(-c4c5ccccc5c(-c5ccccc5)c5ccccc45)c3)cc2)c(C#N)c1 ; N#Cc1ccc2c(c1)c1ccccc1n2-c1ccc(-c2ccc(-c3cccc(-c4c5ccccc5c(-c5ccccc5)c5ccccc45)c3)cc2)cc1C#N ; N#Cc1ccc2c(c1)c1cc(C#N)ccc1n2-c1ccc(-c2ccc(-c3cccc(-c4c5ccccc5c(-c5ccccc5)c5ccccc45)c3)cc2)cc1C#N]

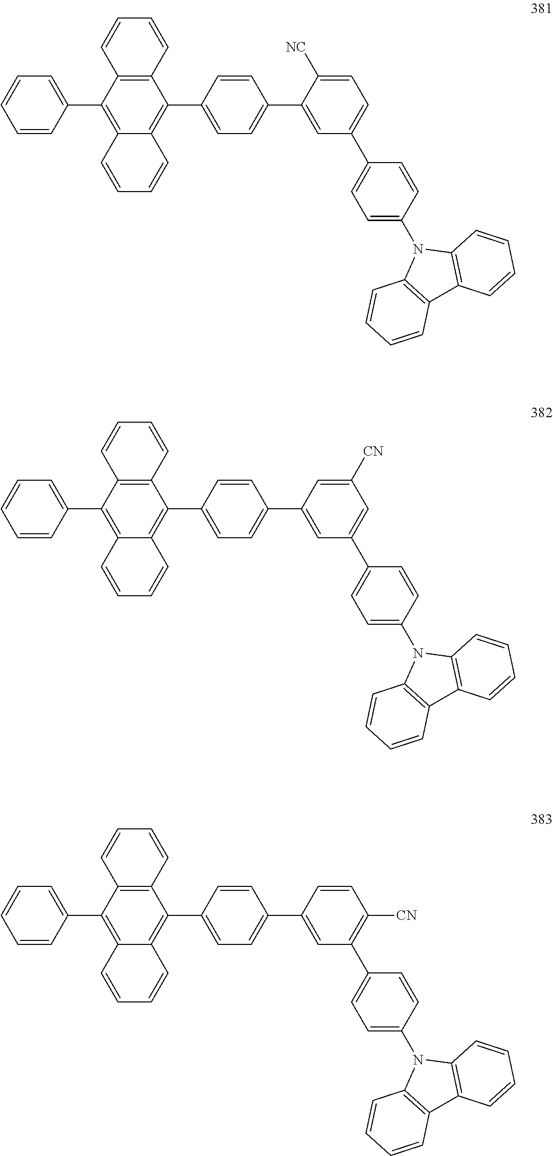 [SMILES: N#Cc1ccc(-c2ccc(-n3c4ccccc4c4ccccc43)cc2)cc1-c1ccc(-c2c3ccccc3c(-c3ccccc3)c3ccccc23)cc1 ; N#Cc1cc(-c2ccc(-c3c4ccccc4c(-c4ccccc4)c4ccccc34)cc2)cc(-c2ccc(-n3c4ccccc4c4ccccc43)cc2)c1 ; N#Cc1ccc(-c2ccc(-c3c4ccccc4c(-c4ccccc4)c4ccccc34)cc2)cc1-c1ccc(-n2c3ccccc3c3ccccc32)cc1]

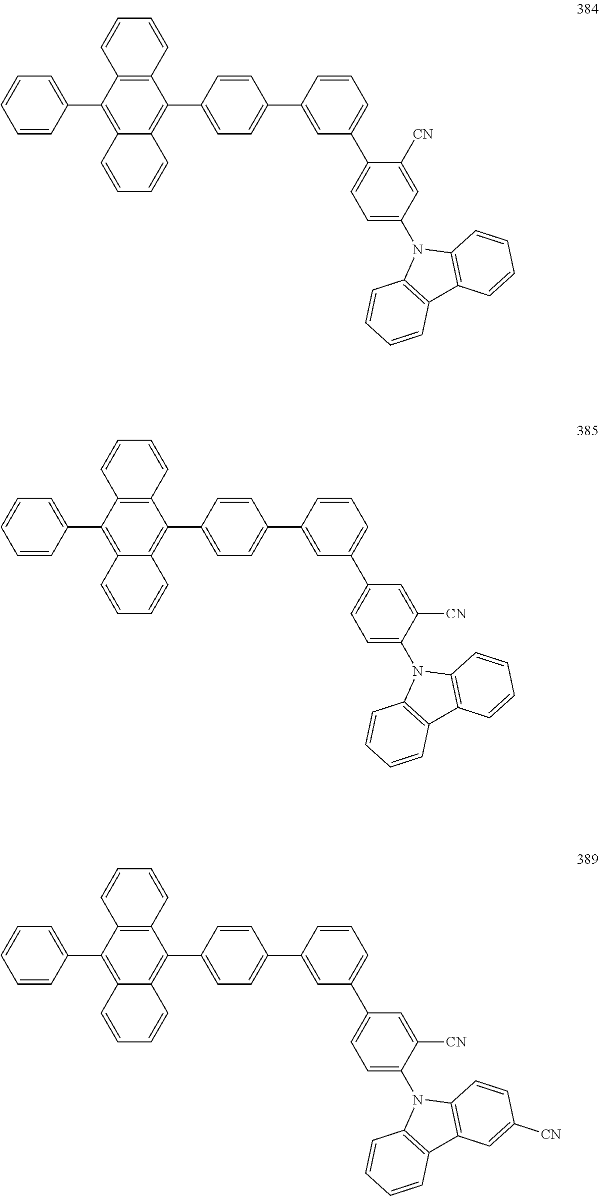 [SMILES: N#Cc1cc(-n2c3ccccc3c3ccccc32)ccc1-c1cccc(-c2ccc(-c3c4ccccc4c(-c4ccccc4)c4ccccc34)cc2)c1 ; N#Cc1cc(-c2cccc(-c3ccc(-c4c5ccccc5c(-c5ccccc5)c5ccccc45)cc3)c2)ccc1-n1c2ccccc2c2ccccc21 ; N#Cc1ccc2c(c1)c1ccccc1n2-c1ccc(-c2cccc(-c3ccc(-c4c5ccccc5c(-c5ccccc5)c5ccccc45)cc3)c2)cc1C#N]

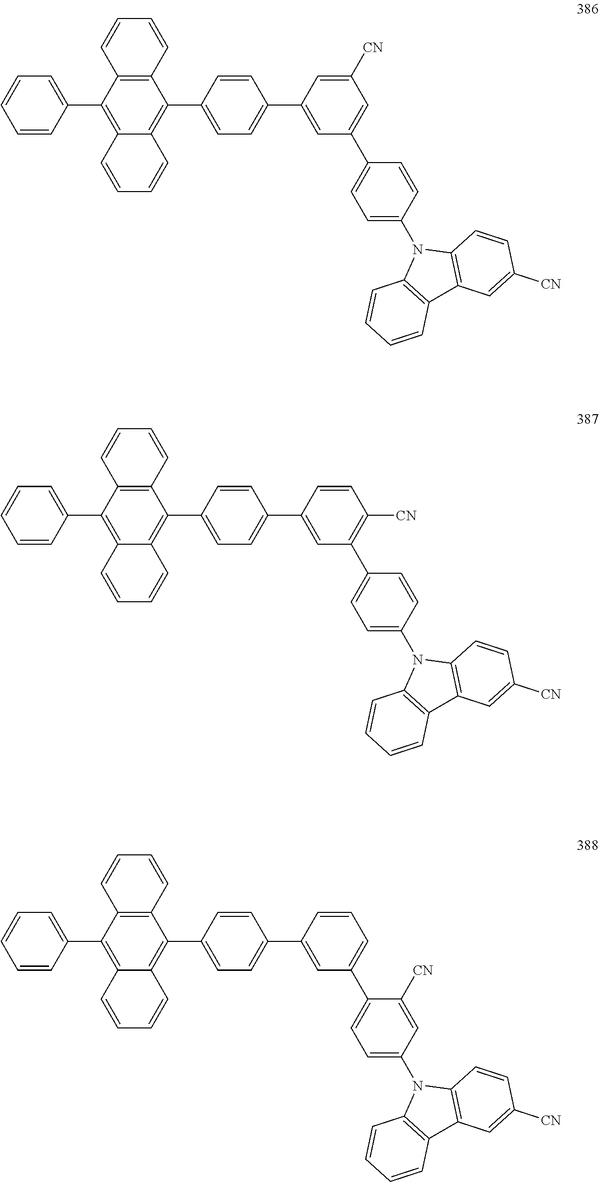 [SMILES: N#Cc1cc(-c2ccc(-c3c4ccccc4c(-c4ccccc4)c4ccccc34)cc2)cc(-c2ccc(-n3c4ccccc4c4cc(C#N)ccc43)cc2)c1 ; N#Cc1ccc2c(c1)c1ccccc1n2-c1ccc(-c2cc(-c3ccc(-c4c5ccccc5c(-c5ccccc5)c5ccccc45)cc3)ccc2C#N)cc1 ; N#Cc1ccc2c(c1)c1ccccc1n2-c1ccc(-c2cccc(-c3ccc(-c4c5ccccc5c(-c5ccccc5)c5ccccc45)cc3)c2)c(C#N)c1]

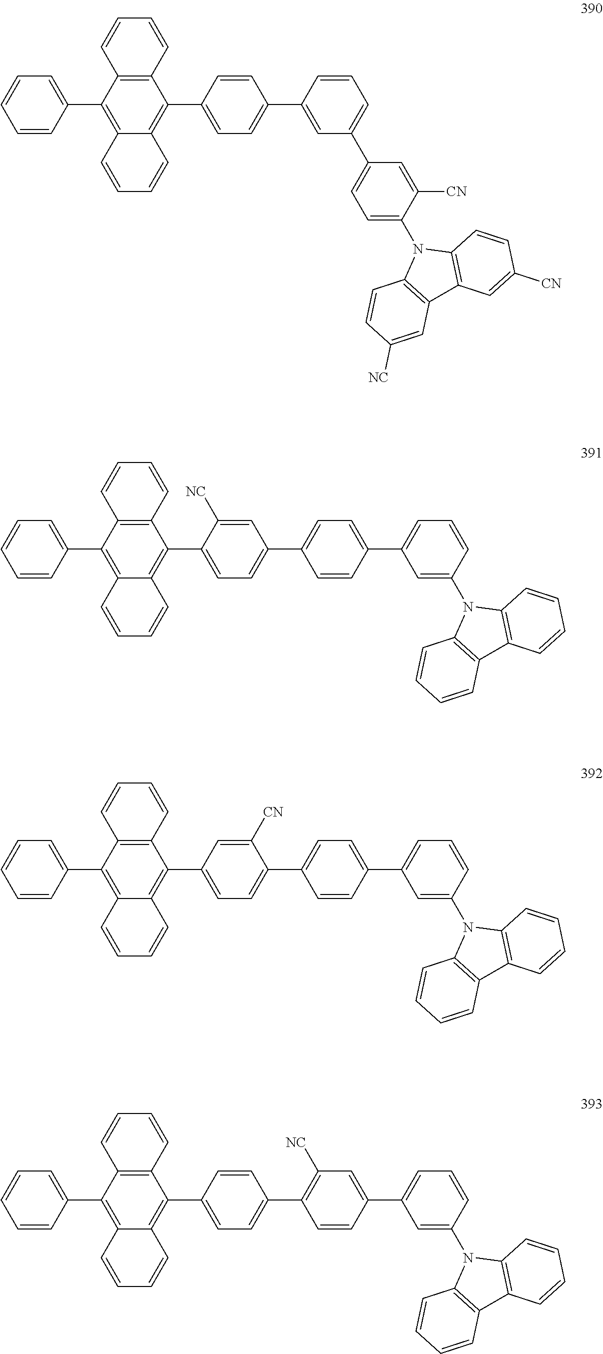 [SMILES: N#Cc1ccc2c(c1)c1cc(C#N)ccc1n2-c1ccc(-c2cccc(-c3ccc(-c4c5ccccc5c(-c5ccccc5)c5ccccc45)cc3)c2)cc1C#N ; N#Cc1cc(-c2ccc(-c3cccc(-n4c5ccccc5c5ccccc54)c3)cc2)ccc1-c1c2ccccc2c(-c2ccccc2)c2ccccc12 ; N#Cc1cc(-c2c3ccccc3c(-c3ccccc3)c3ccccc23)ccc1-c1ccc(-c2cccc(-n3c4ccccc4c4ccccc43)c2)cc1 ; N#Cc1cc(-c2cccc(-n3c4ccccc4c4ccccc43)c2)ccc1-c1ccc(-c2c3ccccc3c(-c3ccccc3)c3ccccc23)cc1]

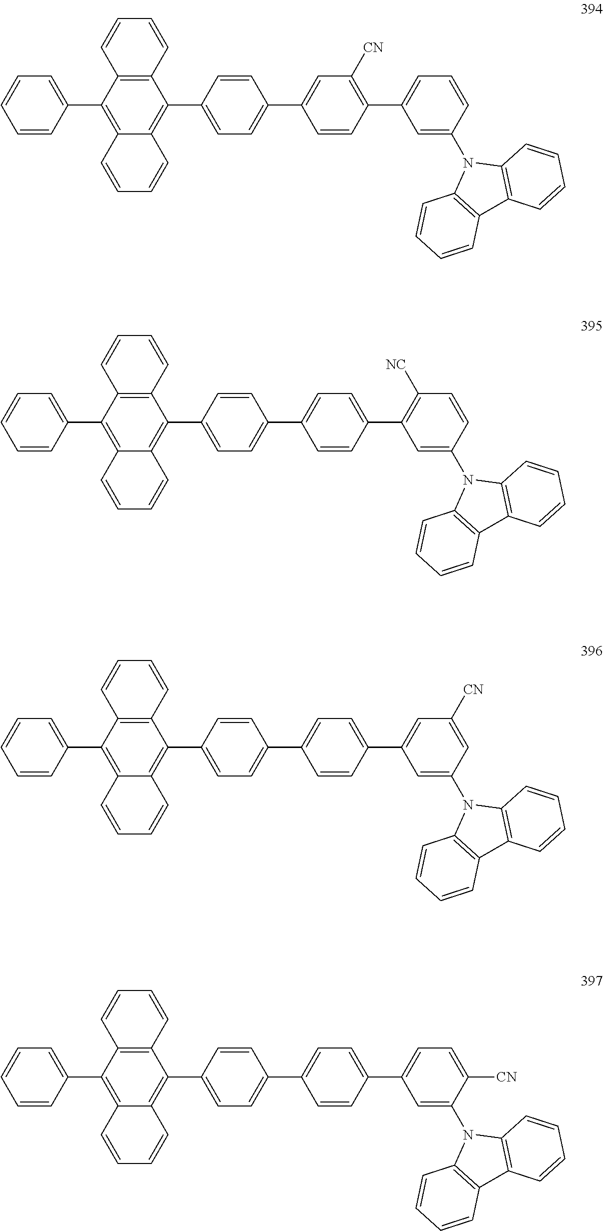 [SMILES: N#Cc1cc(-c2ccc(-c3c4ccccc4c(-c4ccccc4)c4ccccc34)cc2)ccc1-c1cccc(-n2c3ccccc3c3ccccc32)c1 ; N#Cc1ccc(-n2c3ccccc3c3ccccc32)cc1-c1ccc(-c2ccc(-c3c4ccccc4c(-c4ccccc4)c4ccccc34)cc2)cc1 ; N#Cc1cc(-c2ccc(-c3ccc(-c4c5ccccc5c(-c5ccccc5)c5ccccc45)cc3)cc2)cc(-n2c3ccccc3c3ccccc32)c1 ; N#Cc1ccc(-c2ccc(-c3ccc(-c4c5ccccc5c(-c5ccccc5)c5ccccc45)cc3)cc2)cc1-n1c2ccccc2c2ccccc21]

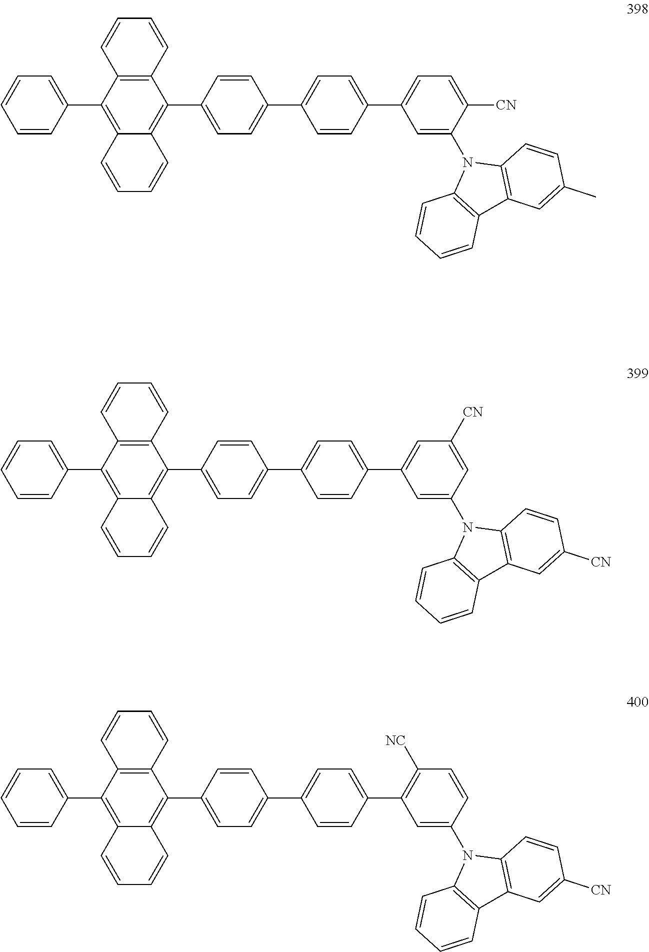 [SMILES: Cc1ccc2c(c1)c1ccccc1n2-c1cc(-c2ccc(-c3ccc(-c4c5ccccc5c(-c5ccccc5)c5ccccc45)cc3)cc2)ccc1C#N ; N#Cc1cc(-c2ccc(-c3ccc(-c4c5ccccc5c(-c5ccccc5)c5ccccc45)cc3)cc2)cc(-n2c3ccccc3c3cc(C#N)ccc32)c1 ; N#Cc1ccc2c(c1)c1ccccc1n2-c1ccc(C#N)c(-c2ccc(-c3ccc(-c4c5ccccc5c(-c5ccccc5)c5ccccc45)cc3)cc2)c1]

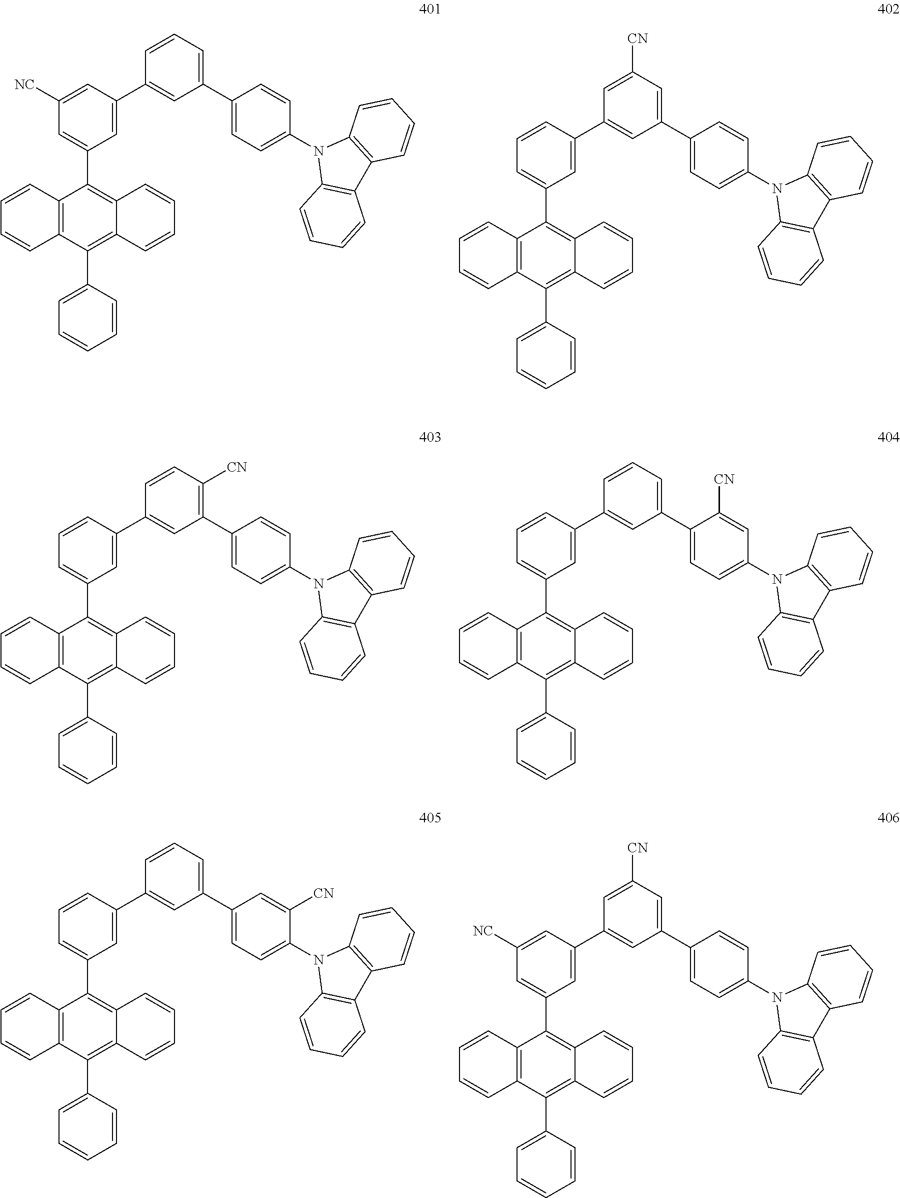

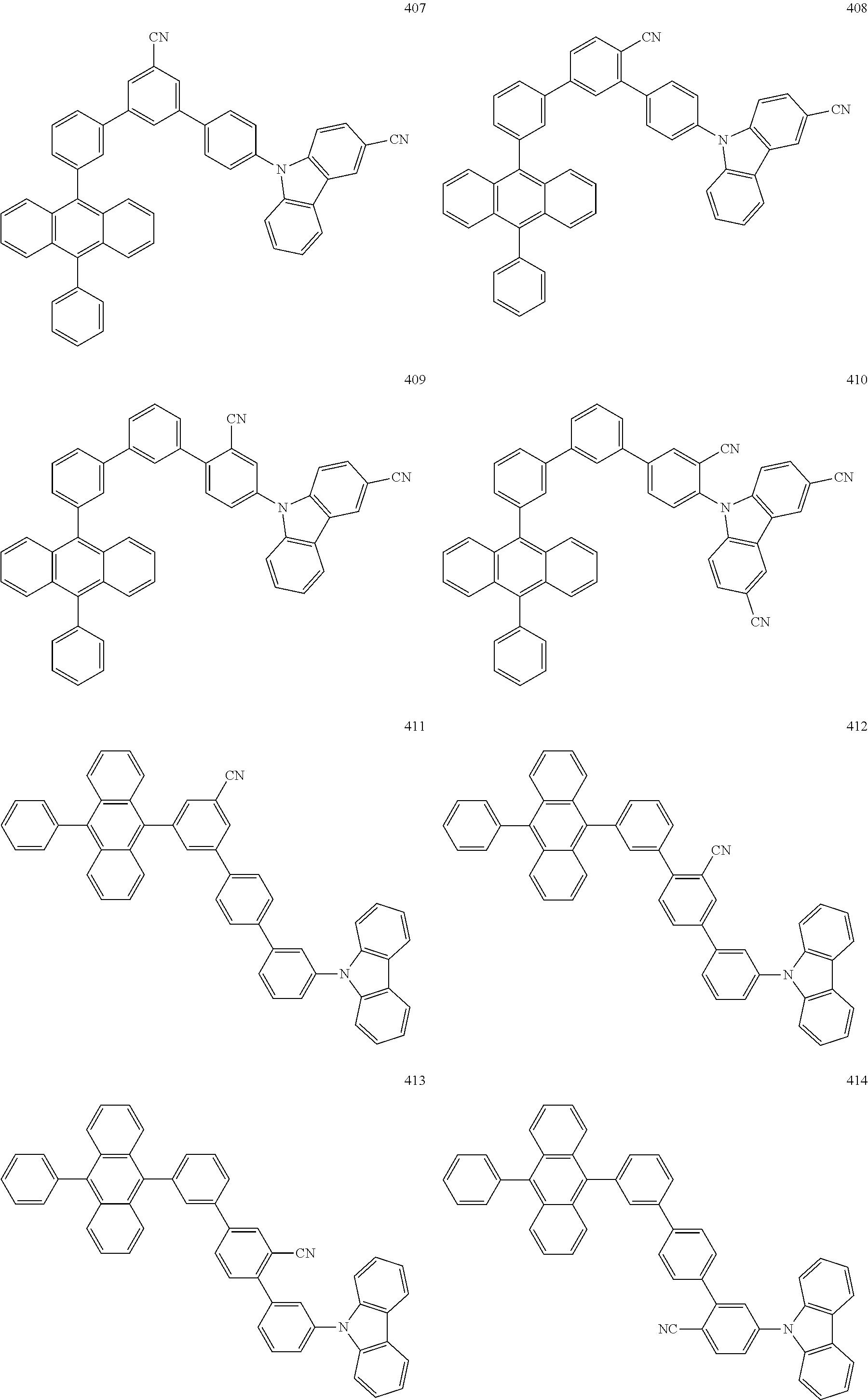

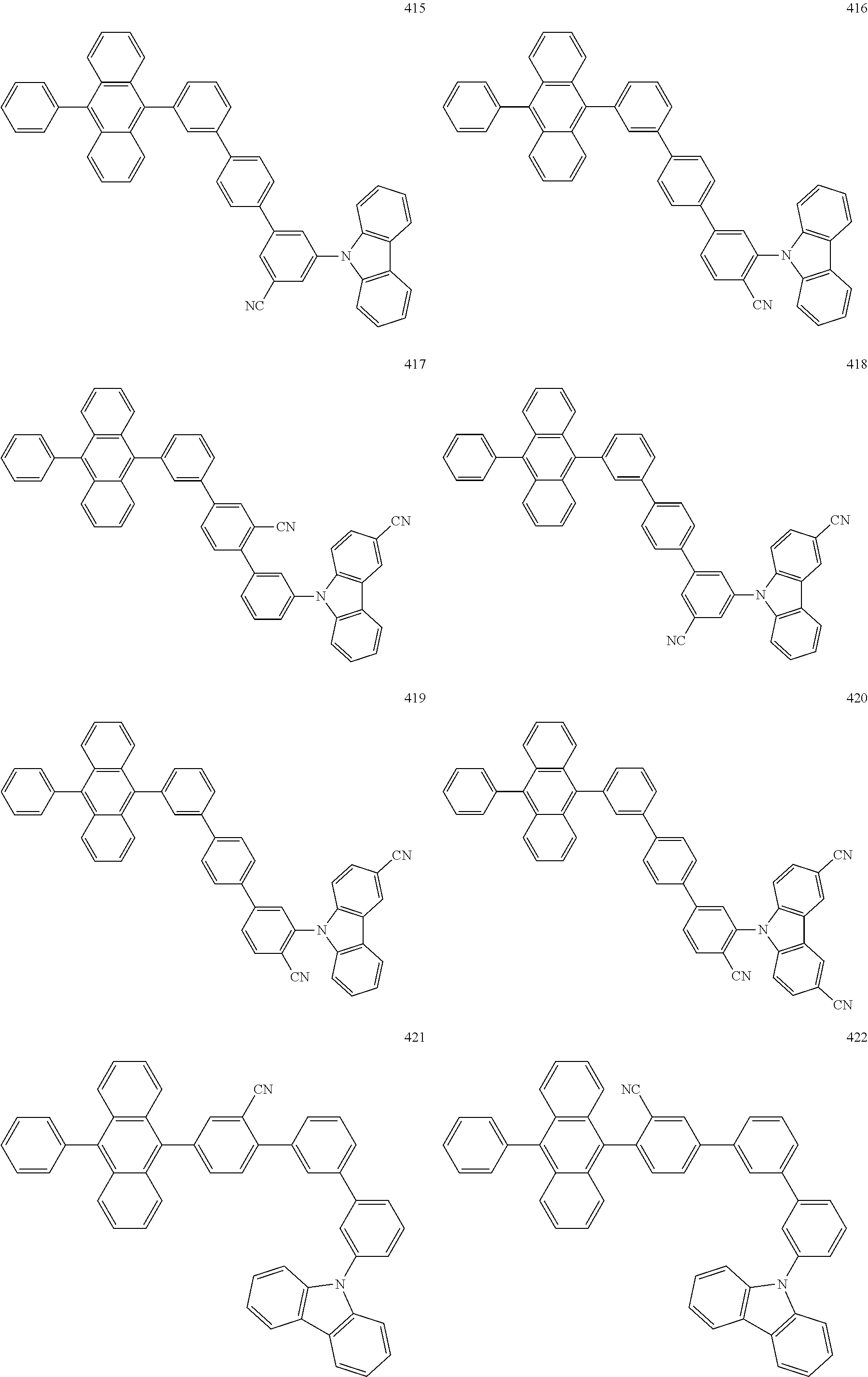

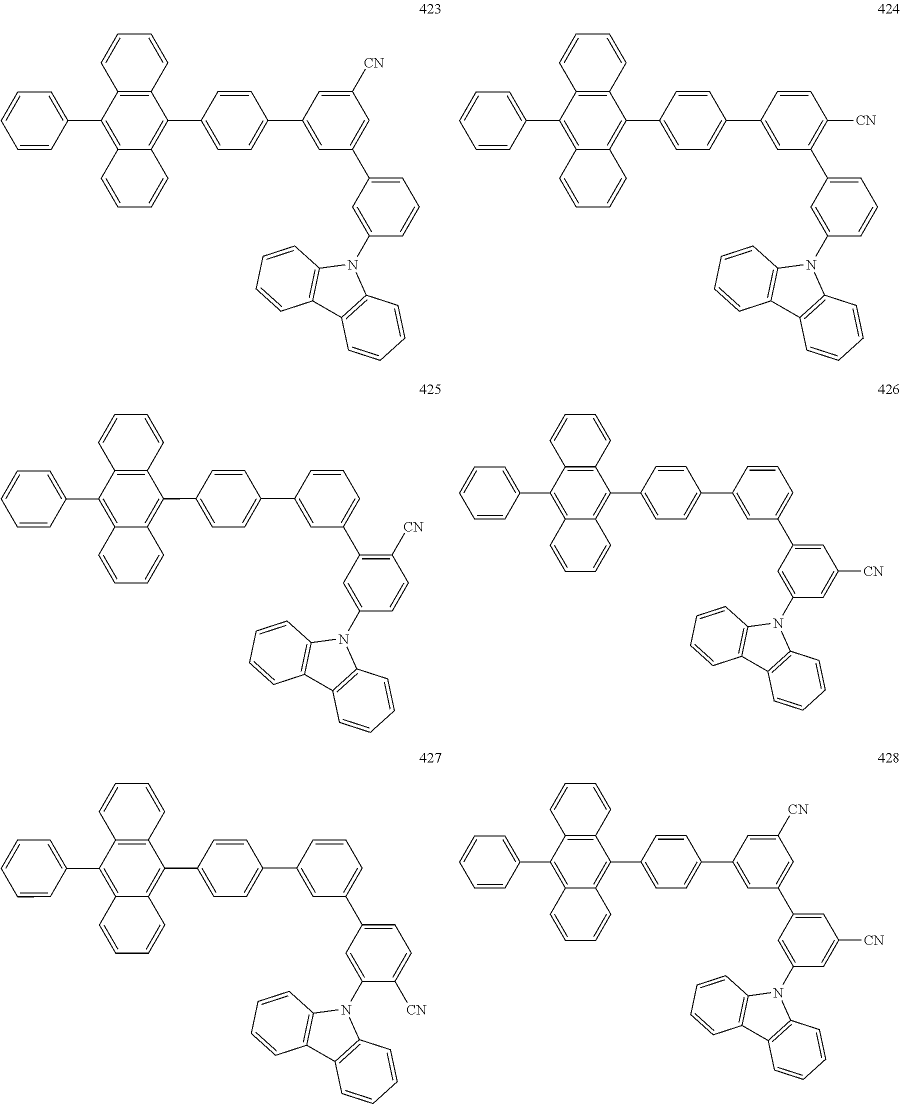

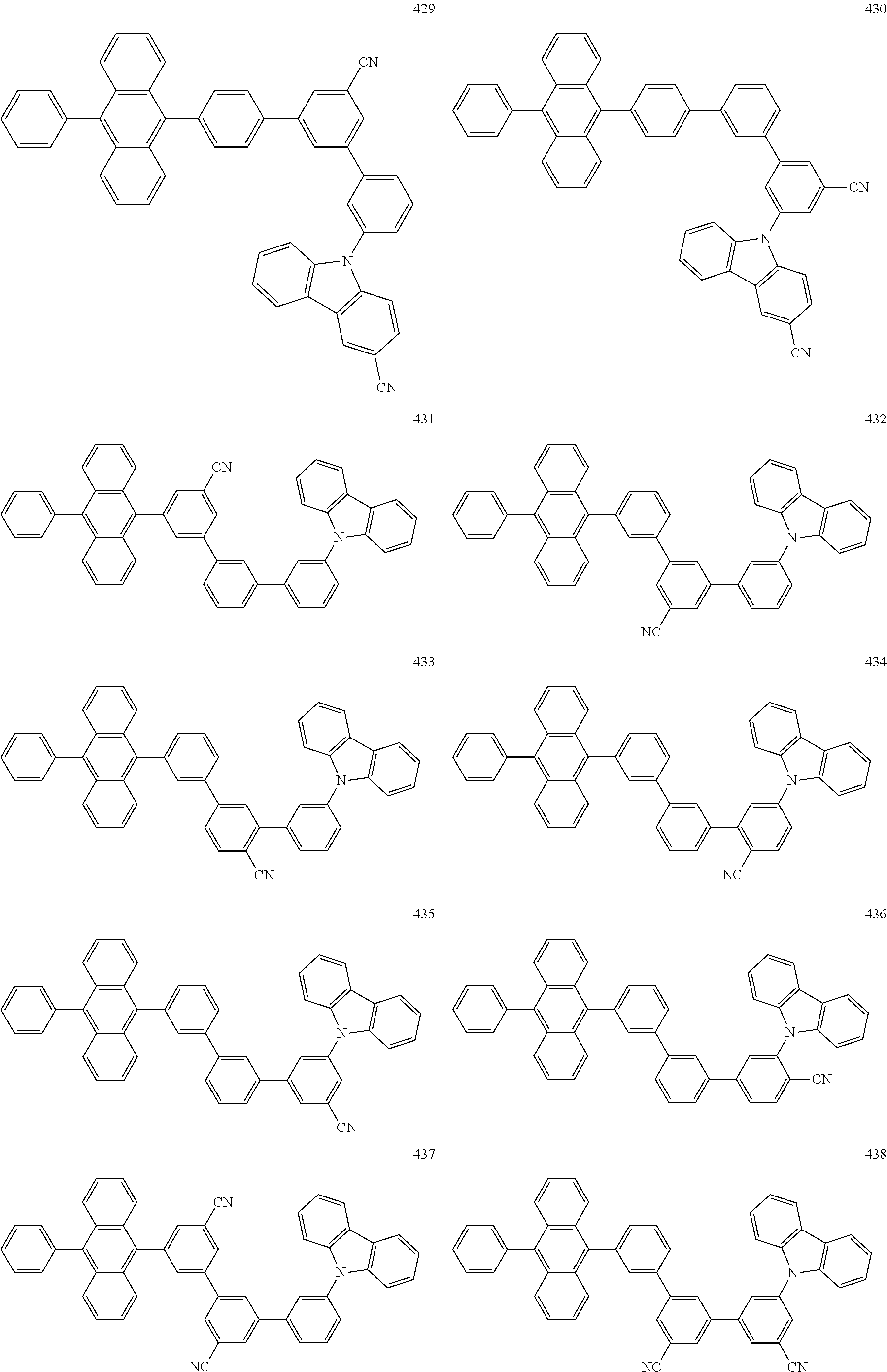

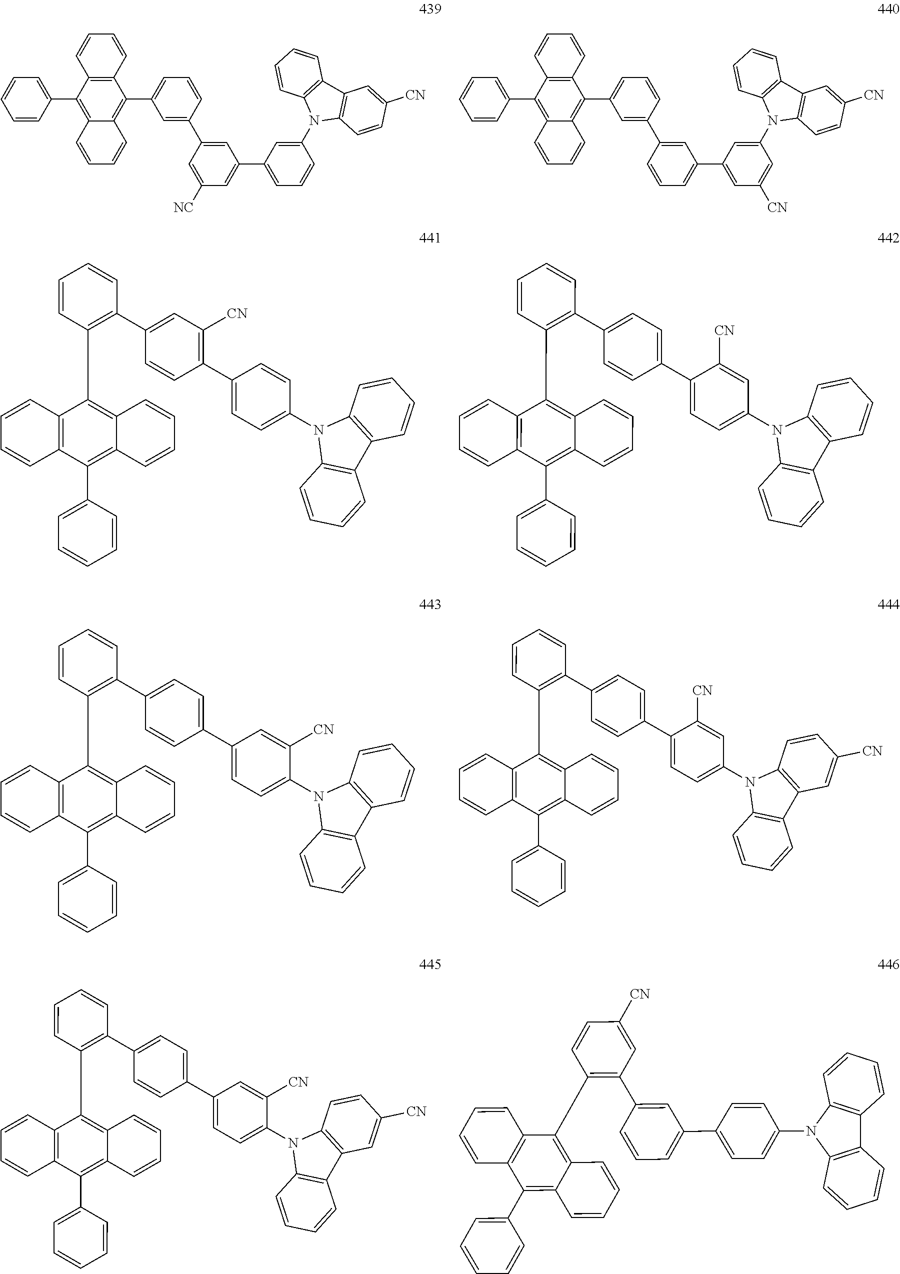

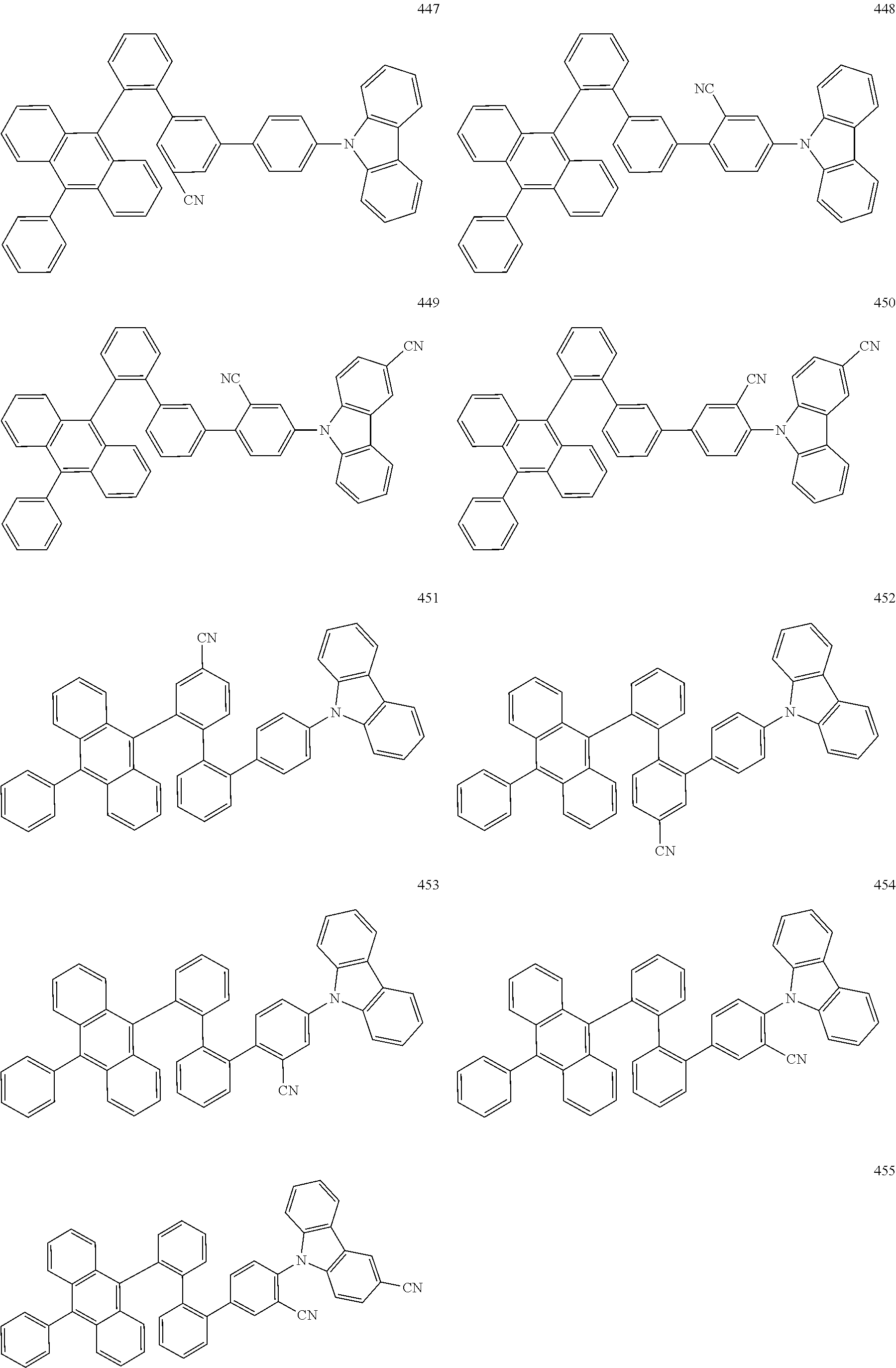

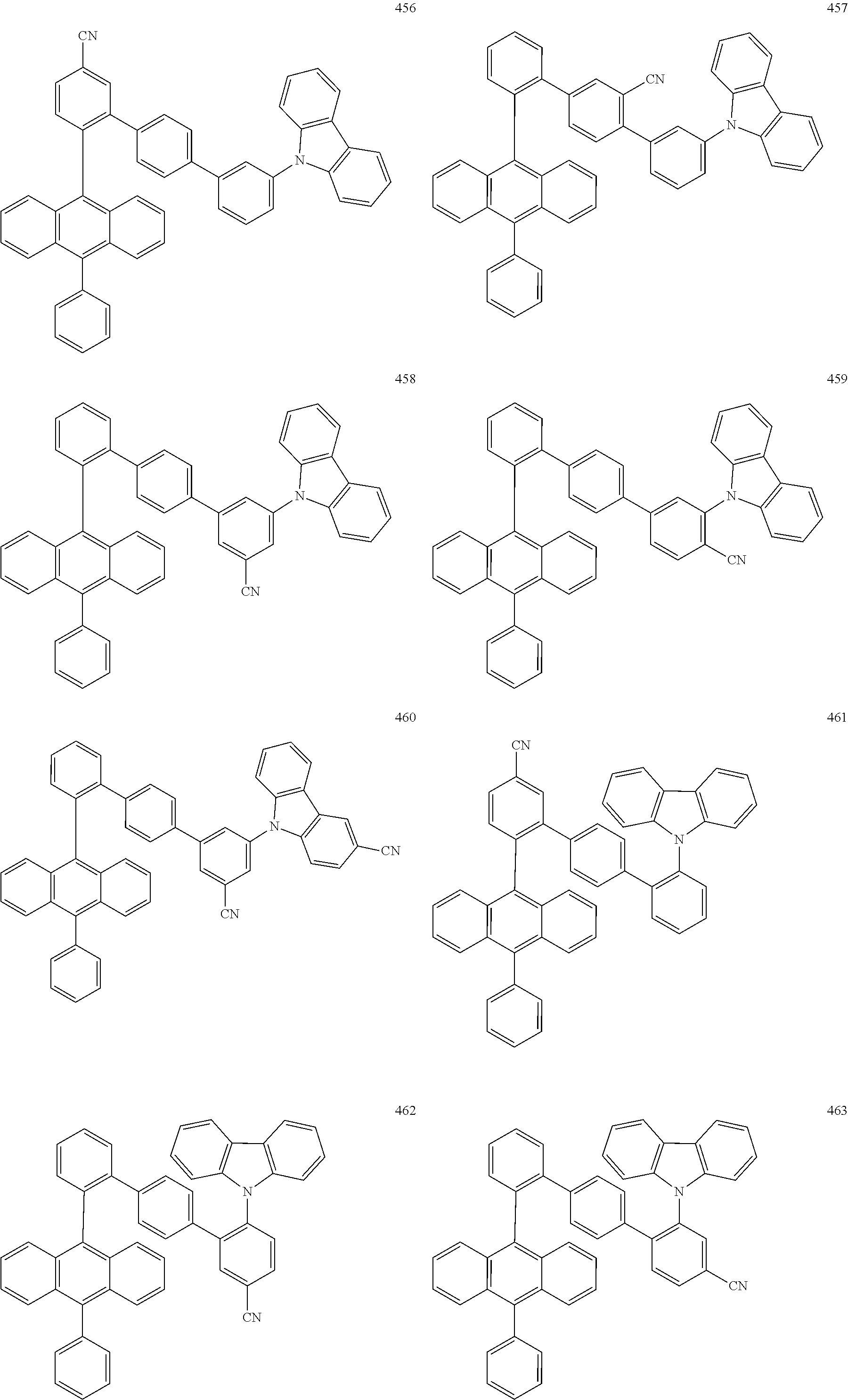

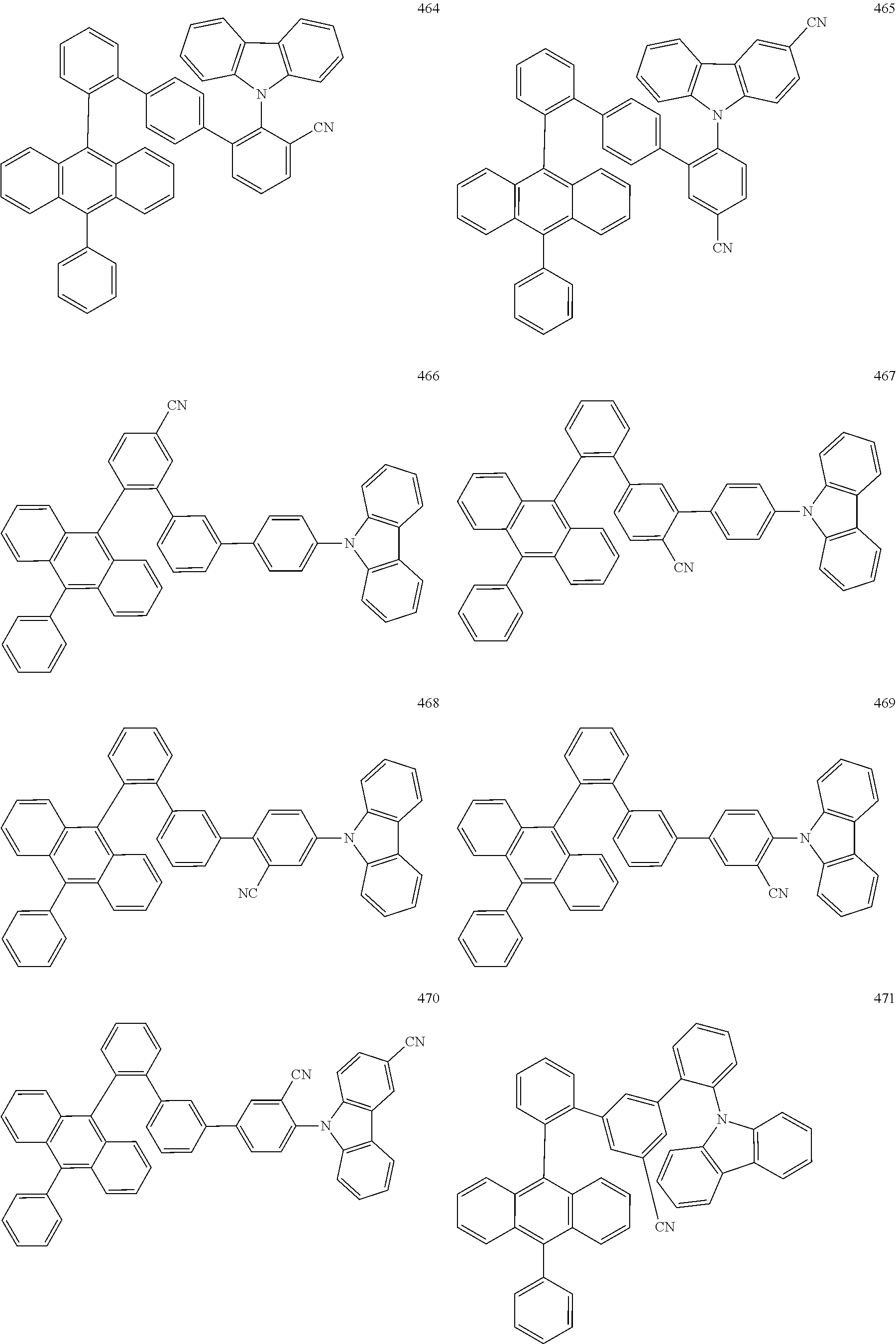

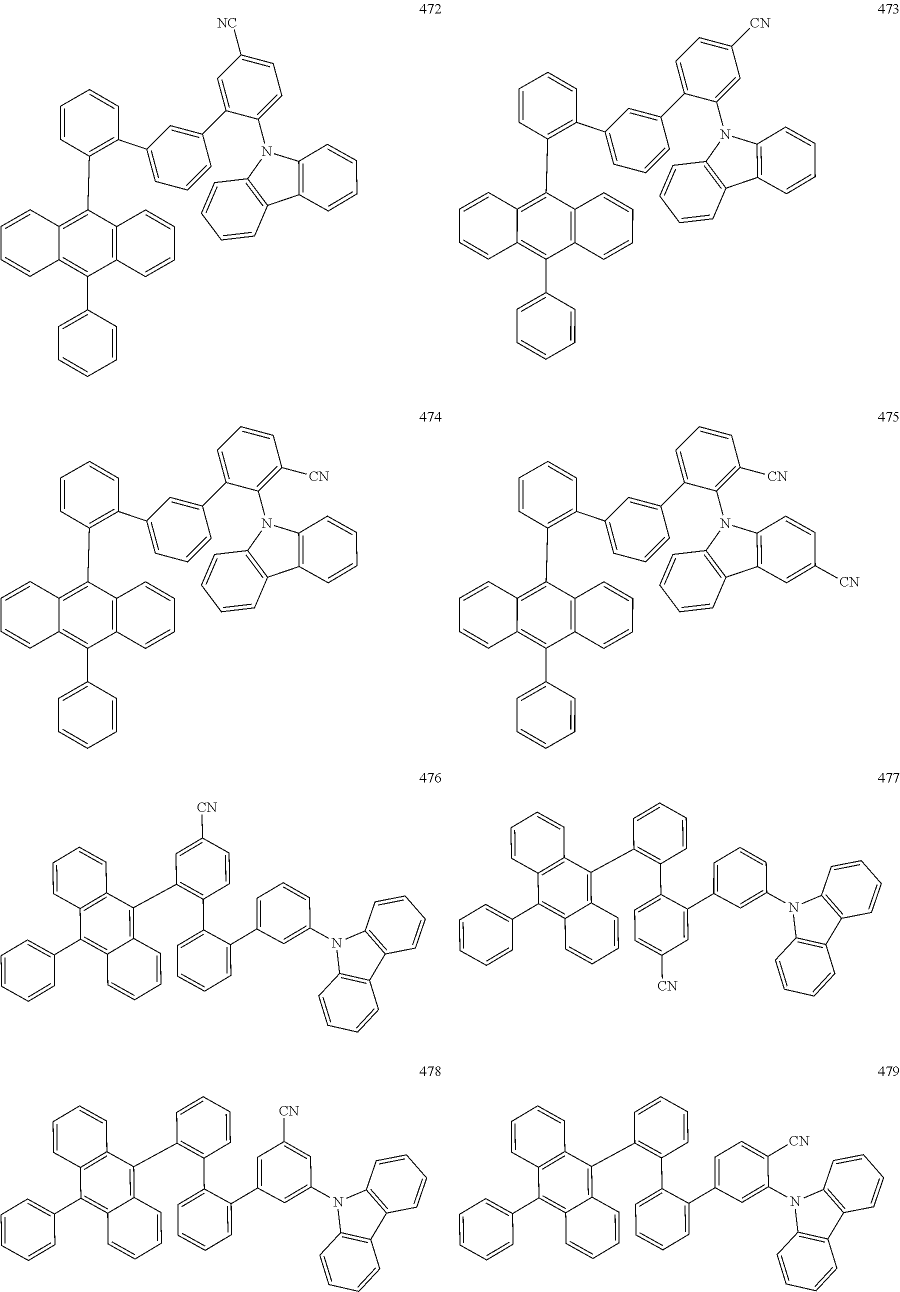

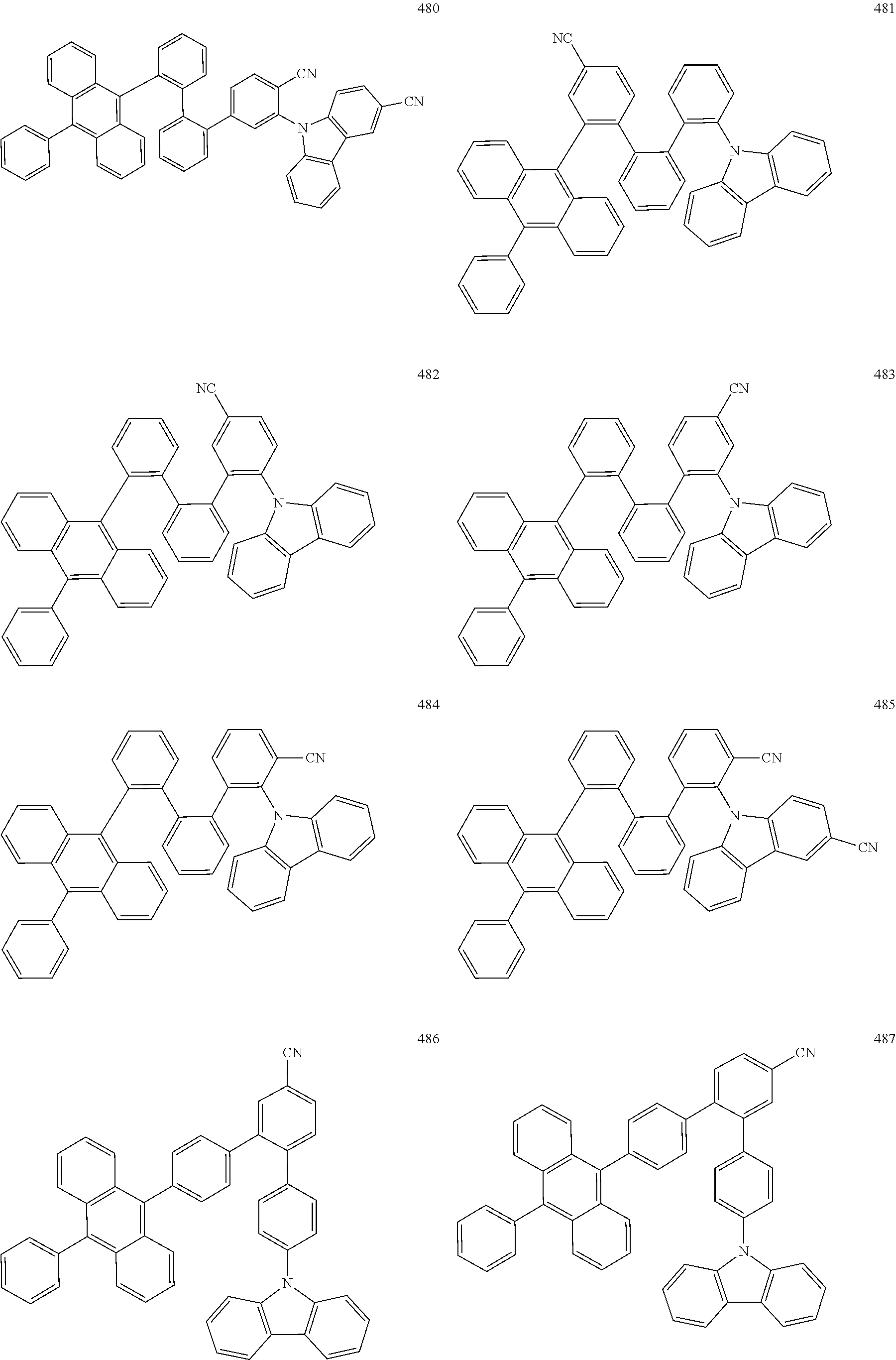

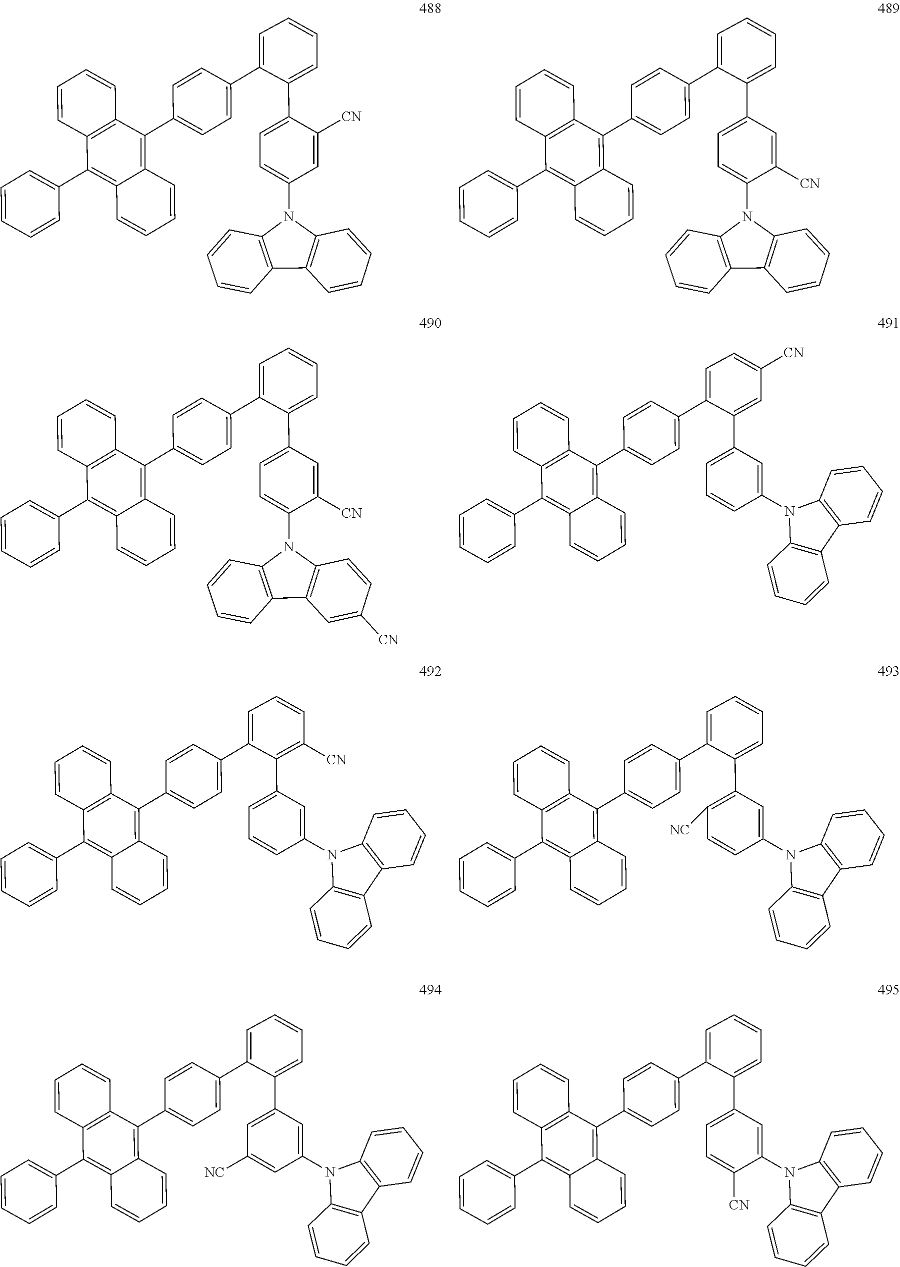

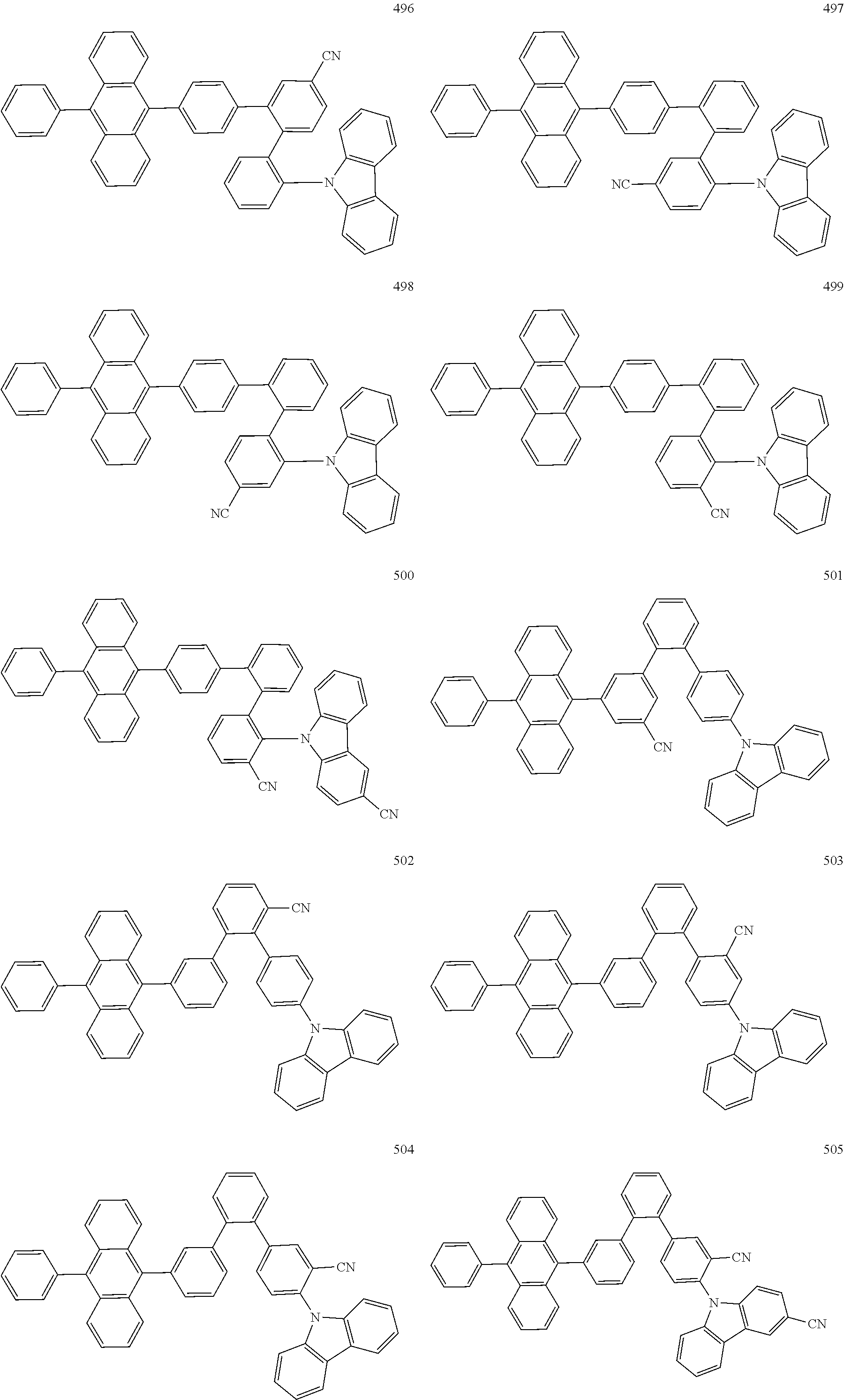

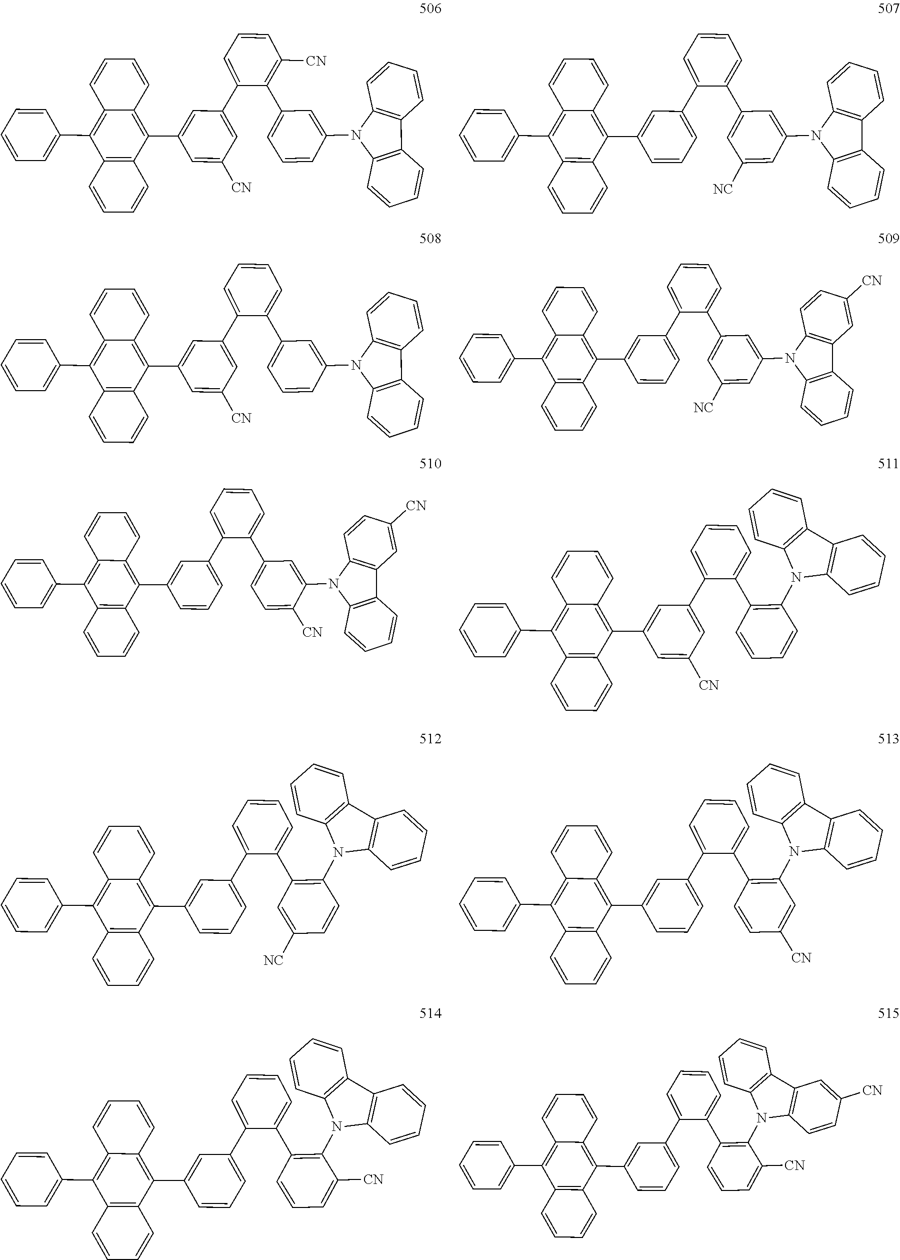

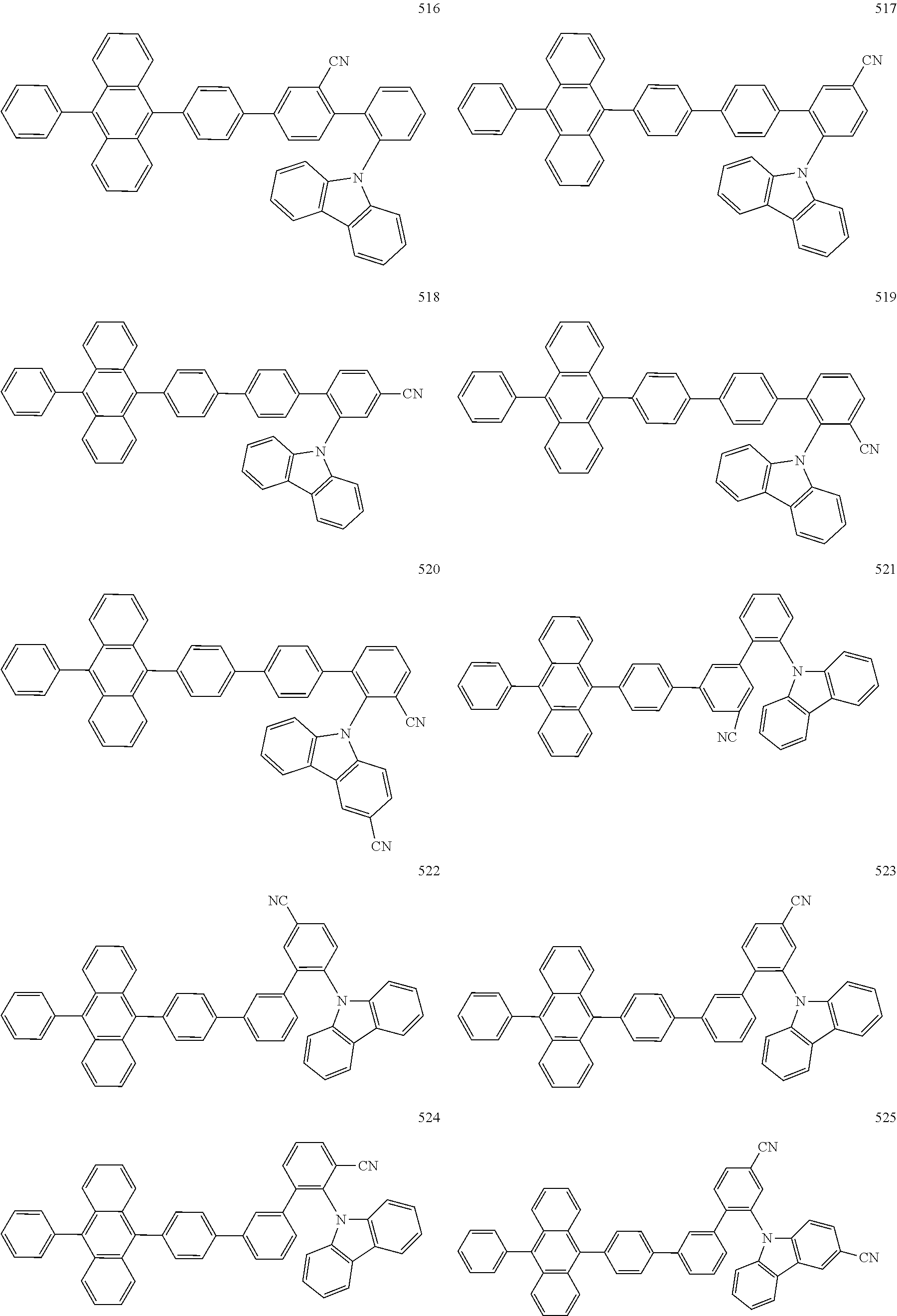

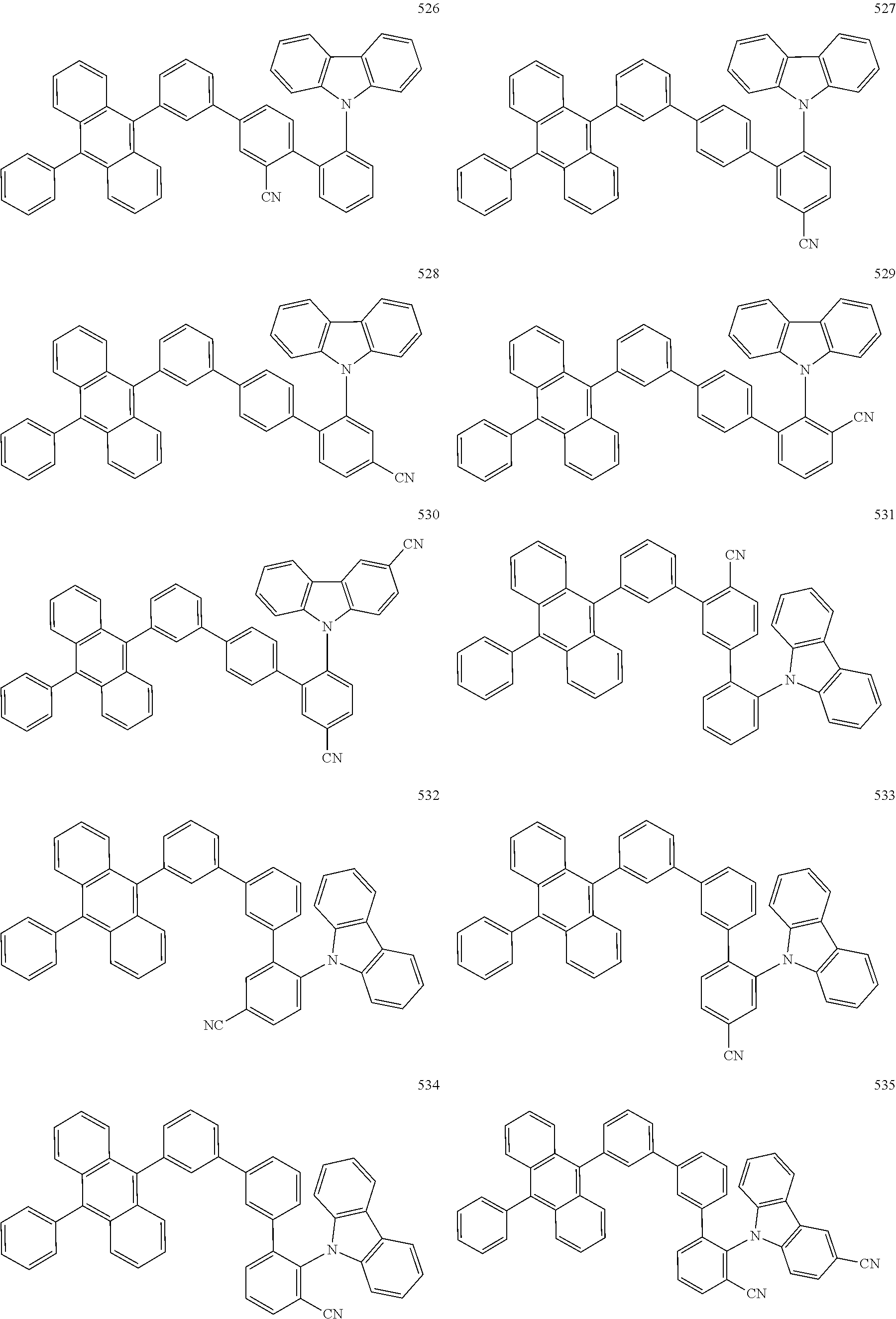

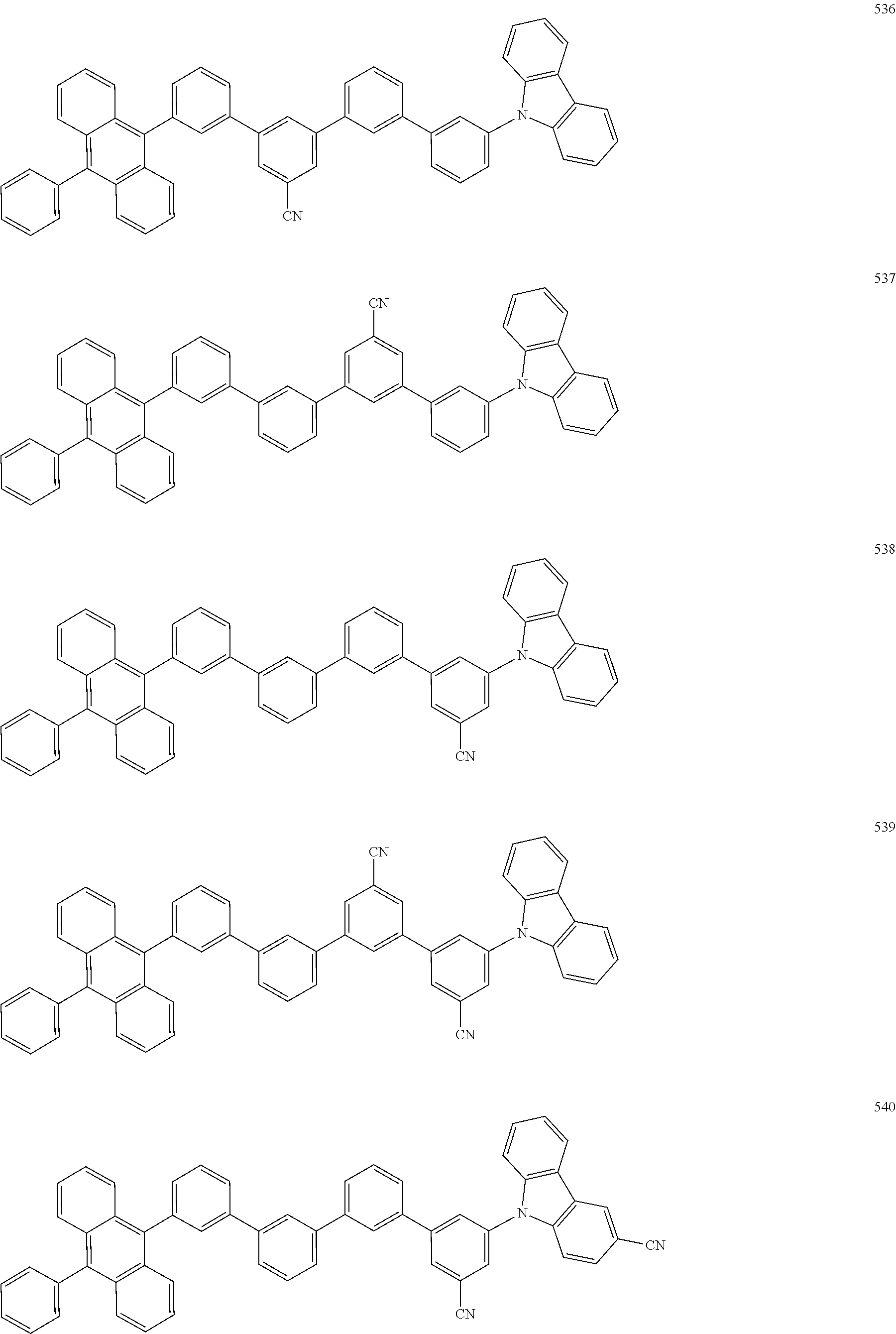

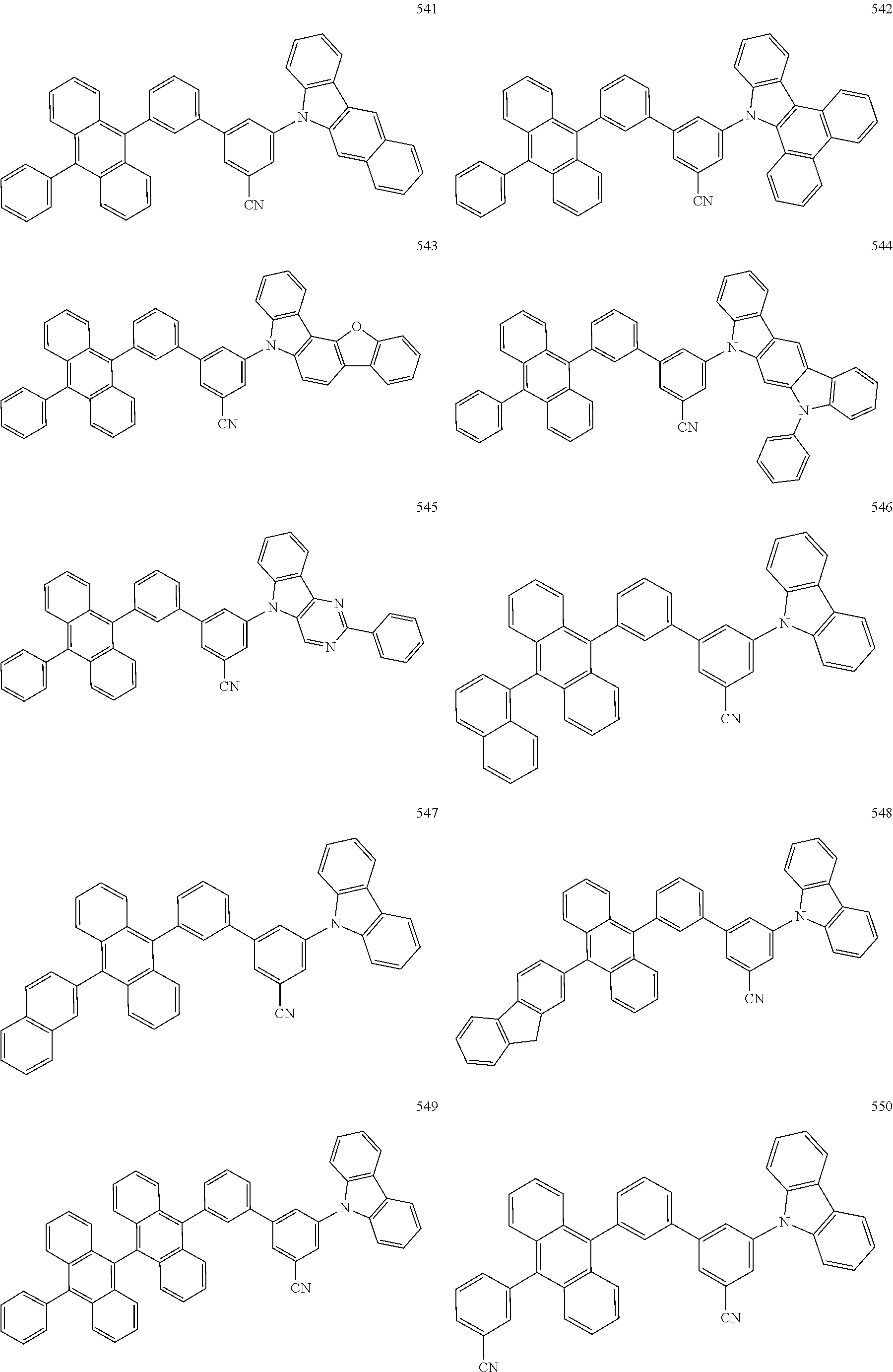

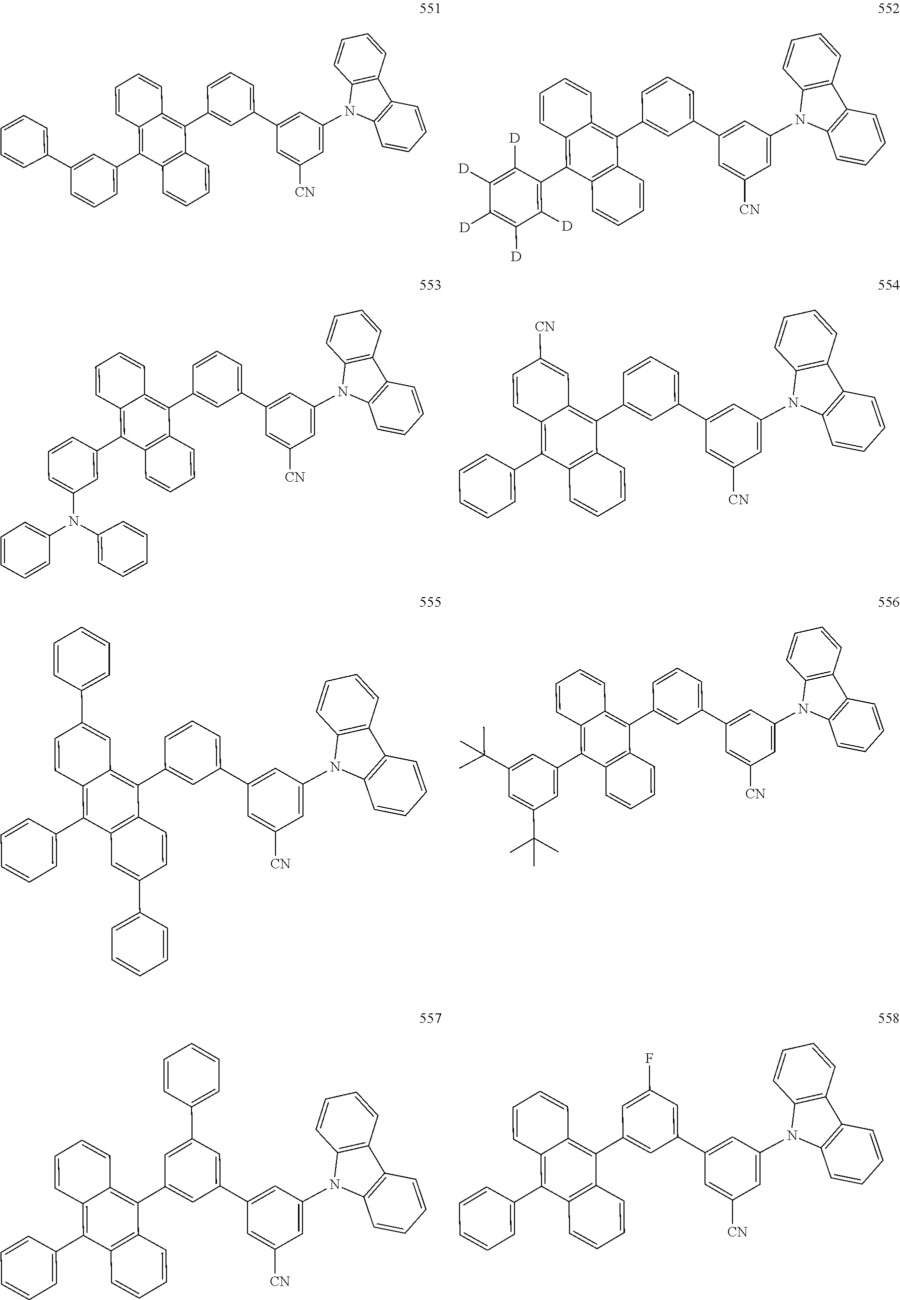

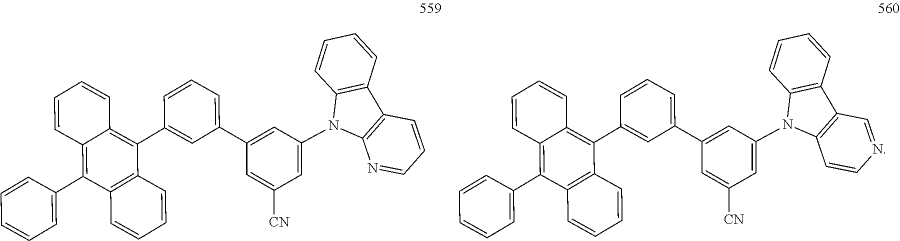 [SMILES: N#Cc1cc(-c2cccc(-c3c4ccccc4c(-c4ccccc4)c4ccccc34)c2)cc(-n2c3ccccc3c3cccnc32)c1 ; N#Cc1cc(-c2cccc(-c3c4ccccc4c(-c4ccccc4)c4ccccc34)c2)cc(-n2c3ccccc3c3cnccc32)c1]

In some embodiments, the fluorescent host may be represented by Formula FH-4:

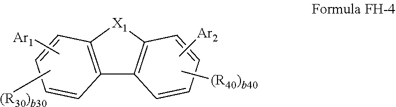

wherein, in Formula FH-4,

X₁ may be O or Se, and

Ar₁ may be a group represented by Formula 1A, and Ar₂ may be a group represented by Formula 1B:

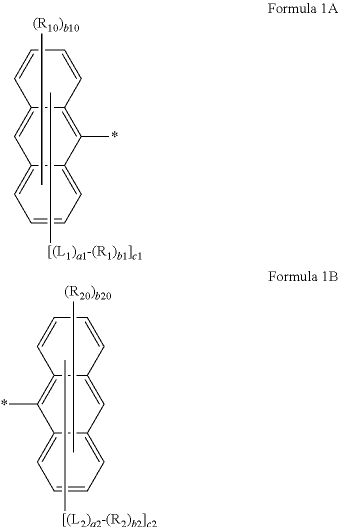

wherein, in Formulae 4 to 6,

L₁ and L₂ may each independently be a substituted or unsubstituted C₅-C₃₀ carbocyclic group or a substituted or unsubstituted C₁-C₃₀ heterocyclic group,

a1 and a2 may be each independently an integer from 0 to 3,

when a1 is 2 or greater, at least two L₁(s) may be identical to or different from each other, and when a2 is 2 or greater, at least two L₂(s) may be identical to or different from each other,

R₁, R₂, R₁₀, R₂₀, R₃₀, and R₄₀ may each independently be hydrogen, deuterium, —F, —Cl, —Br, —I, —SF₅, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C—C₆₀ aryl group, a substituted or unsubstituted C₆-C₆₀ aryloxy group, a substituted or unsubstituted C—C₆₀ arylthio group, a substituted or unsubstituted C₁-C₆₀ heteroaryl group, a substituted or unsubstituted monovalent non-aromatic condensed polycyclic group, a substituted or unsubstituted monovalent non-aromatic condensed heteropolycyclic group, —N(Q₁)(Q₂), —Si(Q₃)(Q₄)(Q₅), —B(Q₆)(Q₇), or —P(═O)(Q₈)(Q₉),

b1 and b2 may each independently be an integer from 1 to 5,

when b1 is 2 or greater, at least two R₁(s) may be identical to or different from each other, and when b2 is 2 or greater, at least two R₂(s) may be identical to or different from each other,

b10 and b20 may each independently be an integer from 1 to 8,

b30 and b40 may each independently be an integer from 1 to 3,

c1 and c2 may each independently be an integer from 1 to 8, and

a sum of b10 and c1 may be 9, and a sum of b20 and c2 may be 9.

In some embodiments, the fluorescent host represented by Formula FH-4 may be Group FH4, but embodiments are not limited thereto:

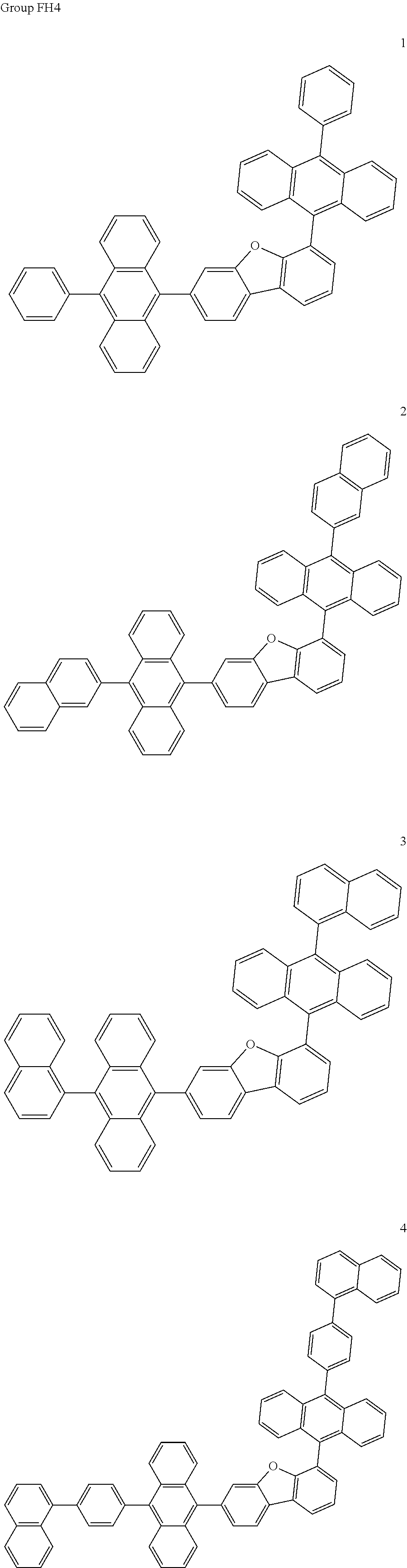 [SMILES: c1ccc(-c2c3ccccc3c(-c3ccc4c(c3)oc3c(-c5c6ccccc6c(-c6ccccc6)c6ccccc56)cccc34)c3ccccc23)cc1 ; c1ccc2cc(-c3c4ccccc4c(-c4ccc5c(c4)oc4c(-c6c7ccccc7c(-c7ccc8ccccc8c7)c7ccccc67)cccc45)c4ccccc34)ccc2c1 ; c1ccc2c(-c3c4ccccc4c(-c4ccc5c(c4)oc4c(-c6c7ccccc7c(-c7cccc8ccccc78)c7ccccc67)cccc45)c4ccccc34)cccc2c1 ; c1ccc2c(-c3ccc(-c4c5ccccc5c(-c5ccc6c(c5)oc5c(-c7c8ccccc8c(-c8ccc(-c9cccc%10ccccc9%10)cc8)c8ccccc78)cccc56)c5ccccc45)cc3)cccc2c1]

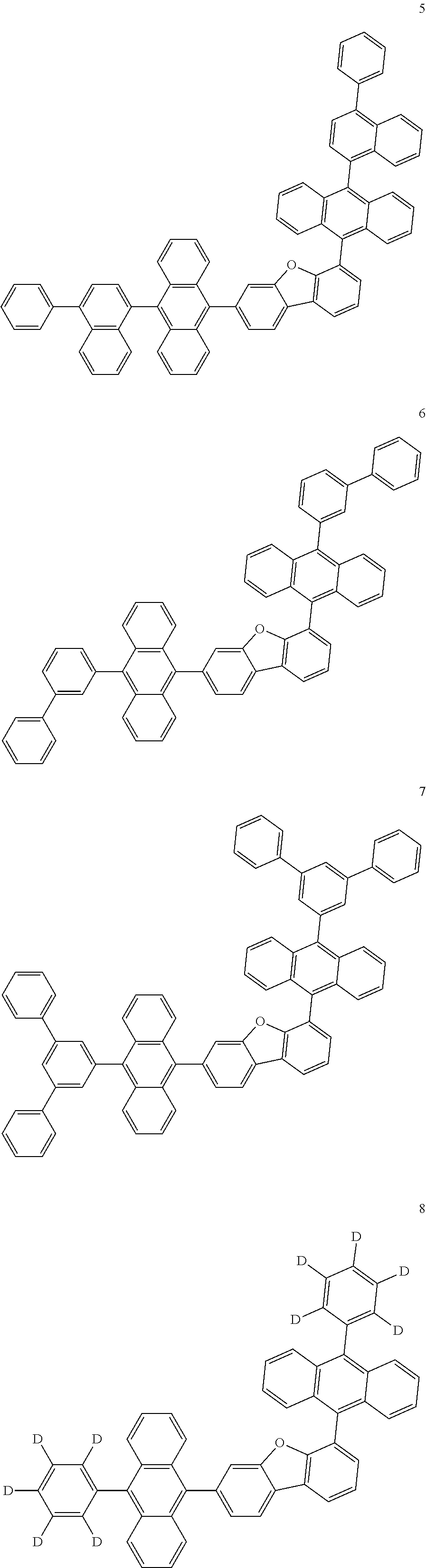

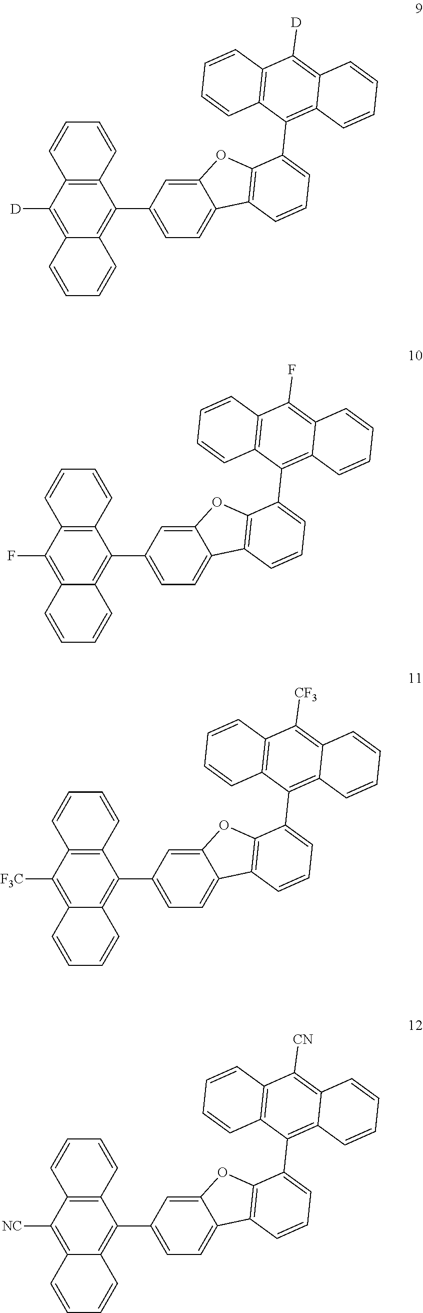 [SMILES: [2H]c1c2ccccc2c(-c2ccc3c(c2)oc2c(-c4c5ccccc5c([2H])c5ccccc45)cccc23)c2ccccc12 ; Fc1c2ccccc2c(-c2ccc3c(c2)oc2c(-c4c5ccccc5c(F)c5ccccc45)cccc23)c2ccccc12 ; FC(F)(F)c1c2ccccc2c(-c2ccc3c(c2)oc2c(-c4c5ccccc5c(C(F)(F)F)c5ccccc45)cccc23)c2ccccc12 ; N#Cc1c2ccccc2c(-c2ccc3c(c2)oc2c(-c4c5ccccc5c(C#N)c5ccccc45)cccc23)c2ccccc12]

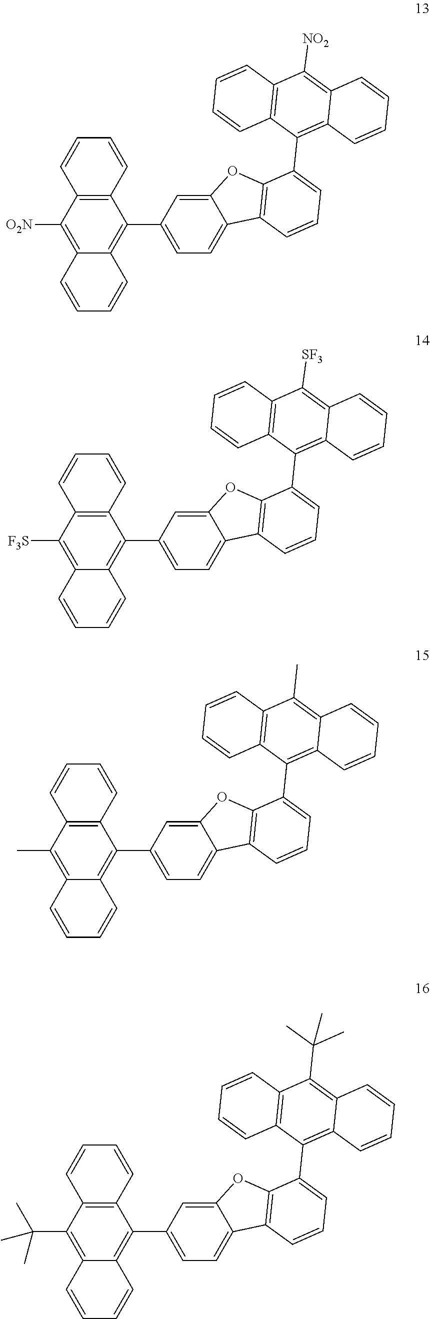

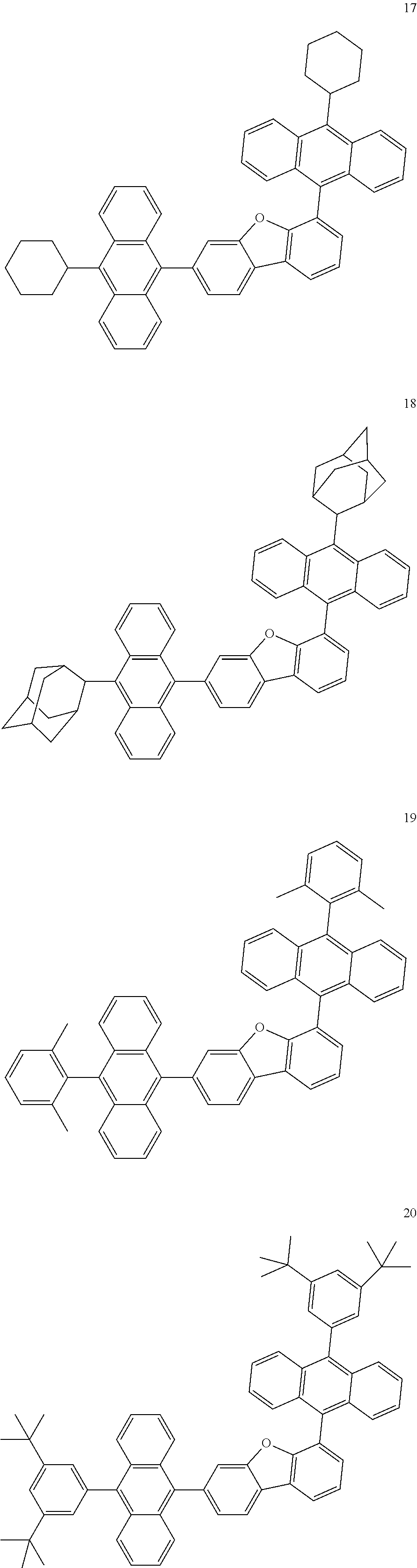 [SMILES: c1ccc2c(C3CCCCC3)c3ccccc3c(-c3ccc4c(c3)oc3c(-c5c6ccccc6c(C6CCCCC6)c6ccccc56)cccc34)c2c1 ; c1ccc2c(C3C4CC5CC(C4)CC3C5)c3ccccc3c(-c3ccc4c(c3)oc3c(-c5c6ccccc6c(C6C7CC8CC(C7)CC6C8)c6ccccc56)cccc34)c2c1 ; Cc1cccc(C)c1-c1c2ccccc2c(-c2ccc3c(c2)oc2c(-c4c5ccccc5c(-c5c(C)cccc5C)c5ccccc45)cccc23)c2ccccc12 ; CC(C)(C)c1cc(-c2c3ccccc3c(-c3ccc4c(c3)oc3c(-c5c6ccccc6c(-c6cc(C(C)(C)C)cc(C(C)(C)C)c6)c6ccccc56)cccc34)c3ccccc23)cc(C(C)(C)C)c1]

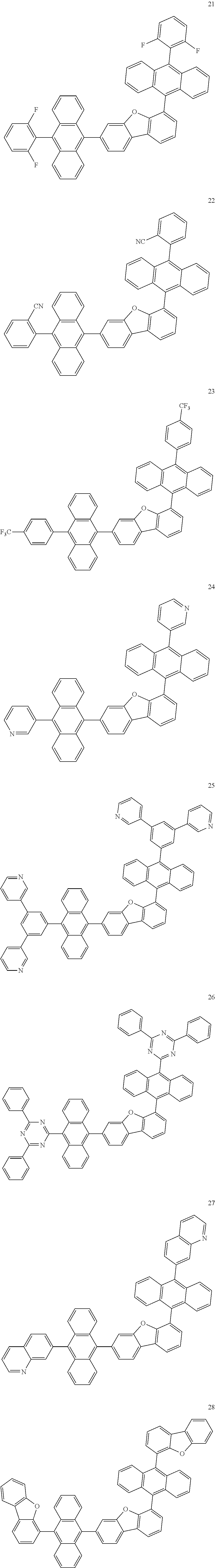

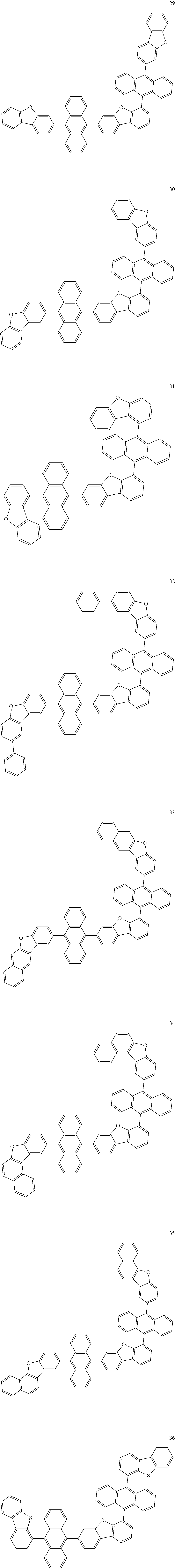

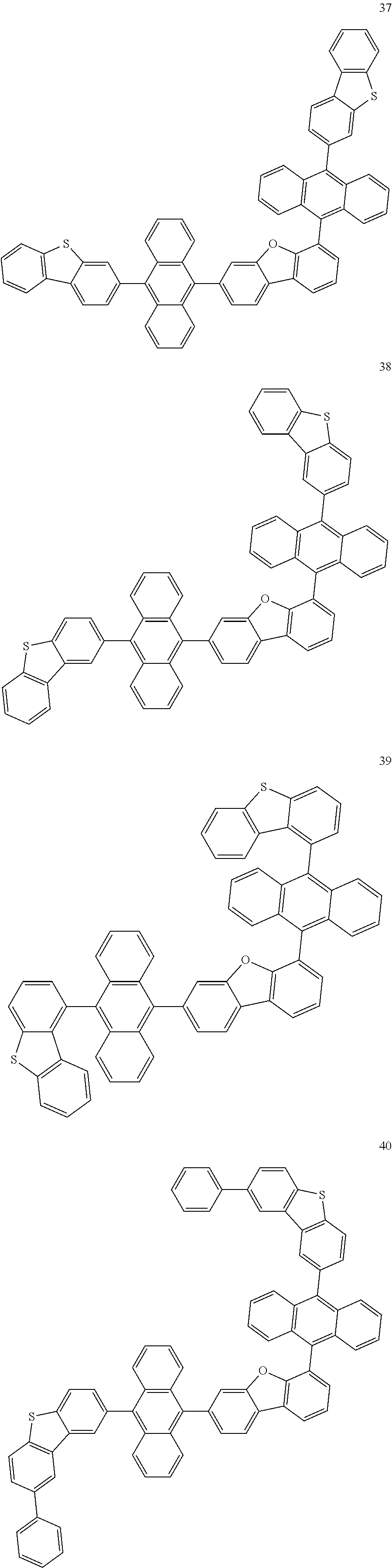 [SMILES: c1ccc2c(c1)sc1cc(-c3c4ccccc4c(-c4ccc5c(c4)oc4c(-c6c7ccccc7c(-c7ccc8c(c7)sc7ccccc78)c7ccccc67)cccc45)c4ccccc34)ccc12 ; c1ccc2c(c1)sc1ccc(-c3c4ccccc4c(-c4ccc5c(c4)oc4c(-c6c7ccccc7c(-c7ccc8sc9ccccc9c8c7)c7ccccc67)cccc45)c4ccccc34)cc12 ; c1ccc2c(c1)sc1cccc(-c3c4ccccc4c(-c4ccc5c(c4)oc4c(-c6c7ccccc7c(-c7cccc8sc9ccccc9c78)c7ccccc67)cccc45)c4ccccc34)c12 ; c1ccc(-c2ccc3sc4ccc(-c5c6ccccc6c(-c6ccc7c(c6)oc6c(-c8c9ccccc9c(-c9ccc%10sc%11ccc(-c%12ccccc%12)cc%11c%10c9)c9ccccc89)cccc67)c6ccccc56)cc4c3c2)cc1]

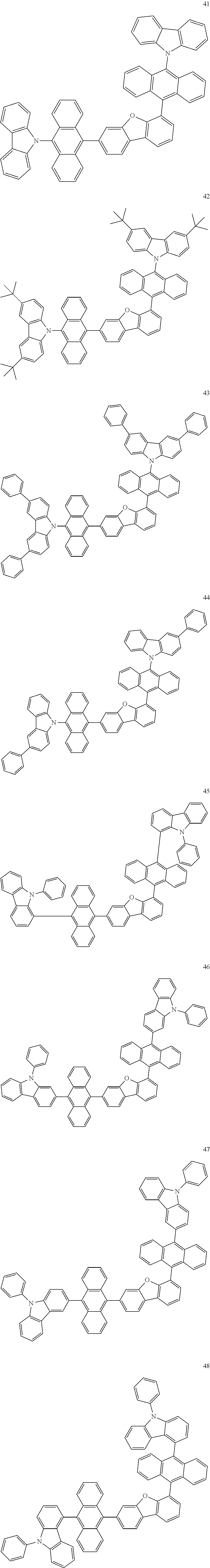

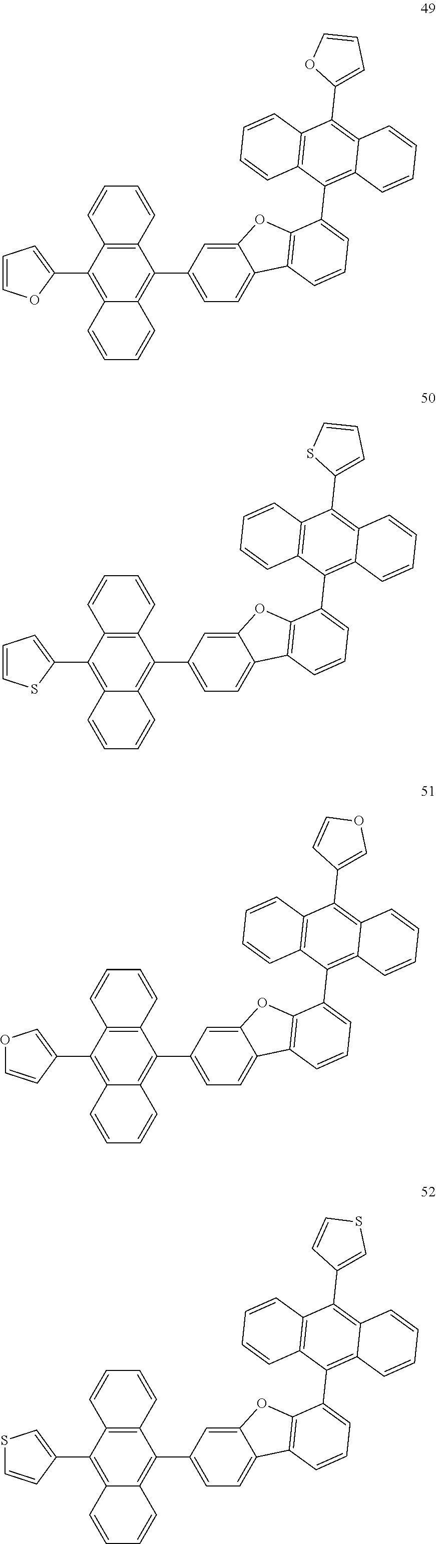 [SMILES: c1coc(-c2c3ccccc3c(-c3ccc4c(c3)oc3c(-c5c6ccccc6c(-c6ccco6)c6ccccc56)cccc34)c3ccccc23)c1 ; c1csc(-c2c3ccccc3c(-c3ccc4c(c3)oc3c(-c5c6ccccc6c(-c6cccs6)c6ccccc56)cccc34)c3ccccc23)c1 ; c1ccc2c(-c3ccc4c(c3)oc3c(-c5c6ccccc6c(-c6ccoc6)c6ccccc56)cccc34)c3ccccc3c(-c3ccoc3)c2c1 ; c1ccc2c(-c3ccc4c(c3)oc3c(-c5c6ccccc6c(-c6ccsc6)c6ccccc56)cccc34)c3ccccc3c(-c3ccsc3)c2c1]

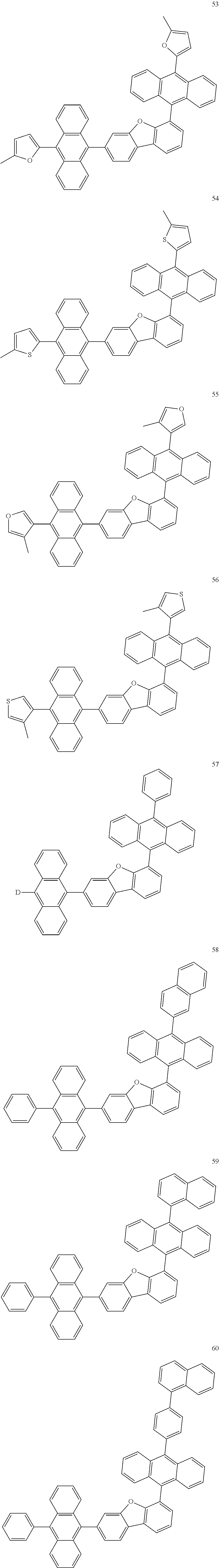

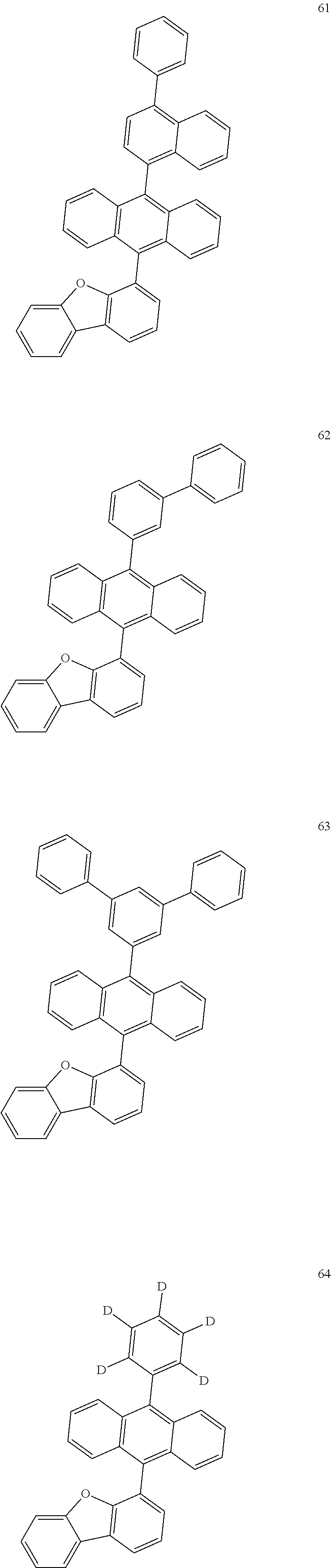 [SMILES: c1ccc(-c2ccc(-c3c4ccccc4c(-c4cccc5c4oc4ccccc45)c4ccccc34)c3ccccc23)cc1 ; c1ccc(-c2cccc(-c3c4ccccc4c(-c4cccc5c4oc4ccccc45)c4ccccc34)c2)cc1 ; c1ccc(-c2cc(-c3ccccc3)cc(-c3c4ccccc4c(-c4cccc5c4oc4ccccc45)c4ccccc34)c2)cc1 ; [2H]c1c([2H])c([2H])c(-c2c3ccccc3c(-c3cccc4c3oc3ccccc34)c3ccccc23)c([2H])c1[2H]]

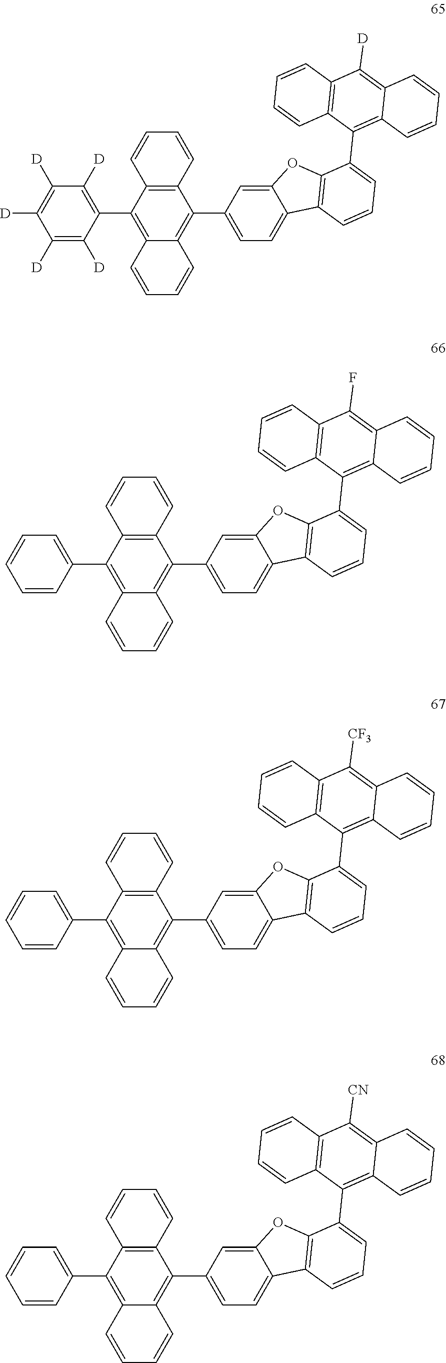 [SMILES: [2H]c1c([2H])c([2H])c(-c2c3ccccc3c(-c3ccc4c(c3)oc3c(-c5c6ccccc6c([2H])c6ccccc56)cccc34)c3ccccc23)c([2H])c1[2H] ; Fc1c2ccccc2c(-c2cccc3c2oc2cc(-c4c5ccccc5c(-c5ccccc5)c5ccccc45)ccc23)c2ccccc12 ; FC(F)(F)c1c2ccccc2c(-c2cccc3c2oc2cc(-c4c5ccccc5c(-c5ccccc5)c5ccccc45)ccc23)c2ccccc12 ; N#Cc1c2ccccc2c(-c2cccc3c2oc2cc(-c4c5ccccc5c(-c5ccccc5)c5ccccc45)ccc23)c2ccccc12]

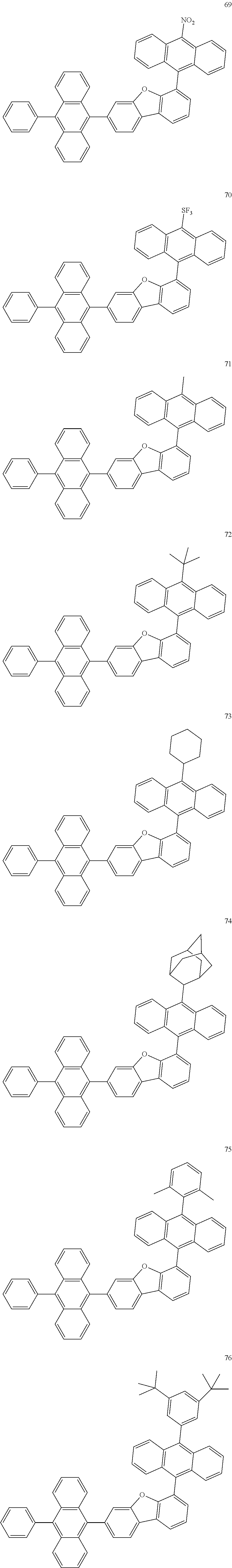

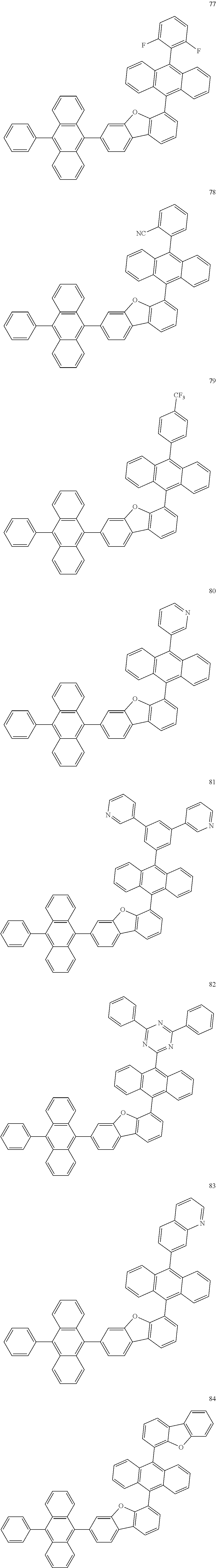

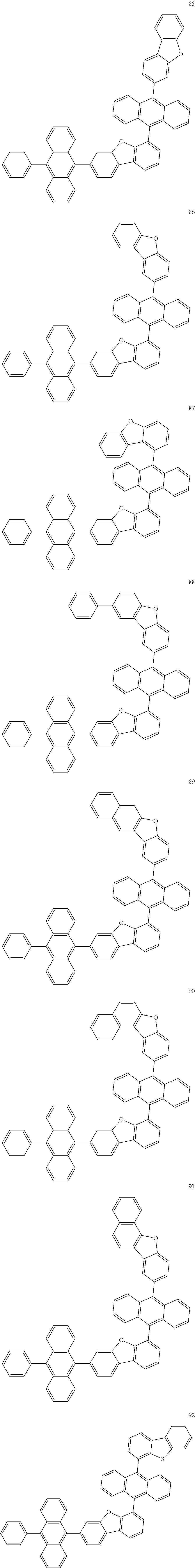

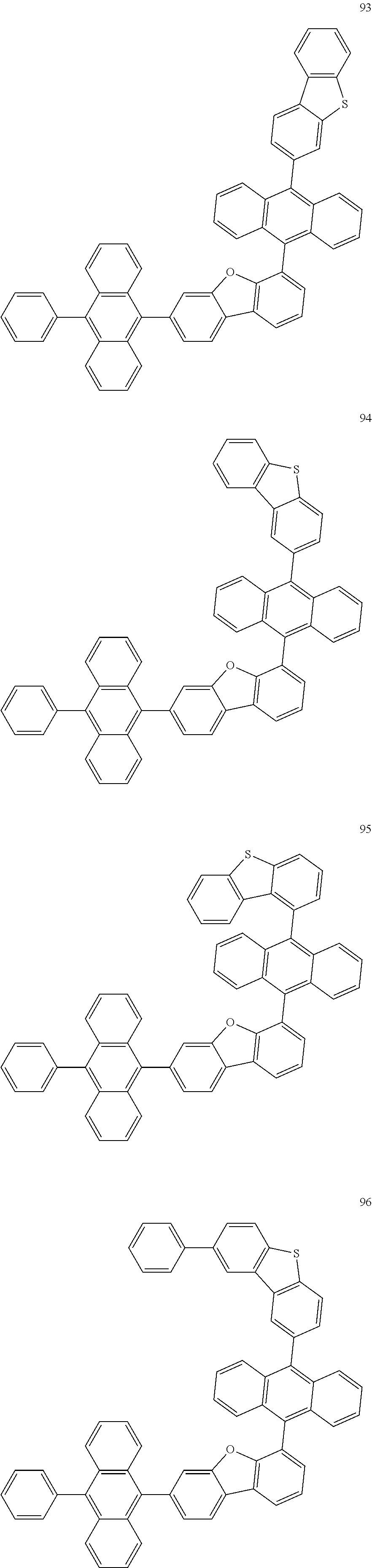 [SMILES: c1ccc(-c2c3ccccc3c(-c3ccc4c(c3)oc3c(-c5c6ccccc6c(-c6ccc7c(c6)sc6ccccc67)c6ccccc56)cccc34)c3ccccc23)cc1 ; c1ccc(-c2c3ccccc3c(-c3ccc4c(c3)oc3c(-c5c6ccccc6c(-c6ccc7sc8ccccc8c7c6)c6ccccc56)cccc34)c3ccccc23)cc1 ; c1ccc(-c2c3ccccc3c(-c3ccc4c(c3)oc3c(-c5c6ccccc6c(-c6cccc7sc8ccccc8c67)c6ccccc56)cccc34)c3ccccc23)cc1 ; c1ccc(-c2ccc3sc4ccc(-c5c6ccccc6c(-c6cccc7c6oc6cc(-c8c9ccccc9c(-c9ccccc9)c9ccccc89)ccc67)c6ccccc56)cc4c3c2)cc1]

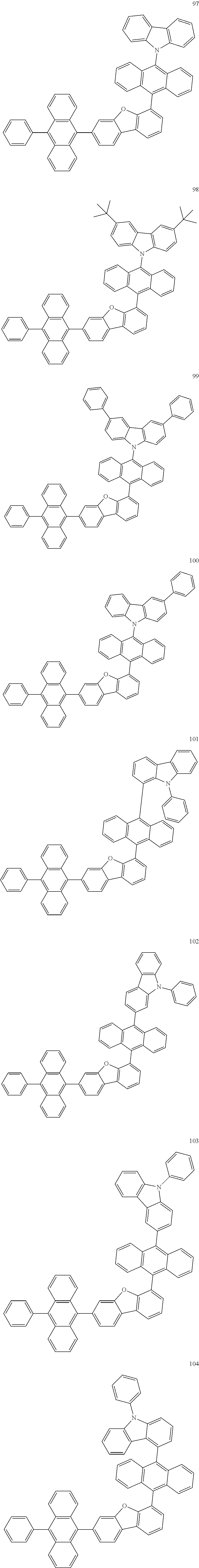

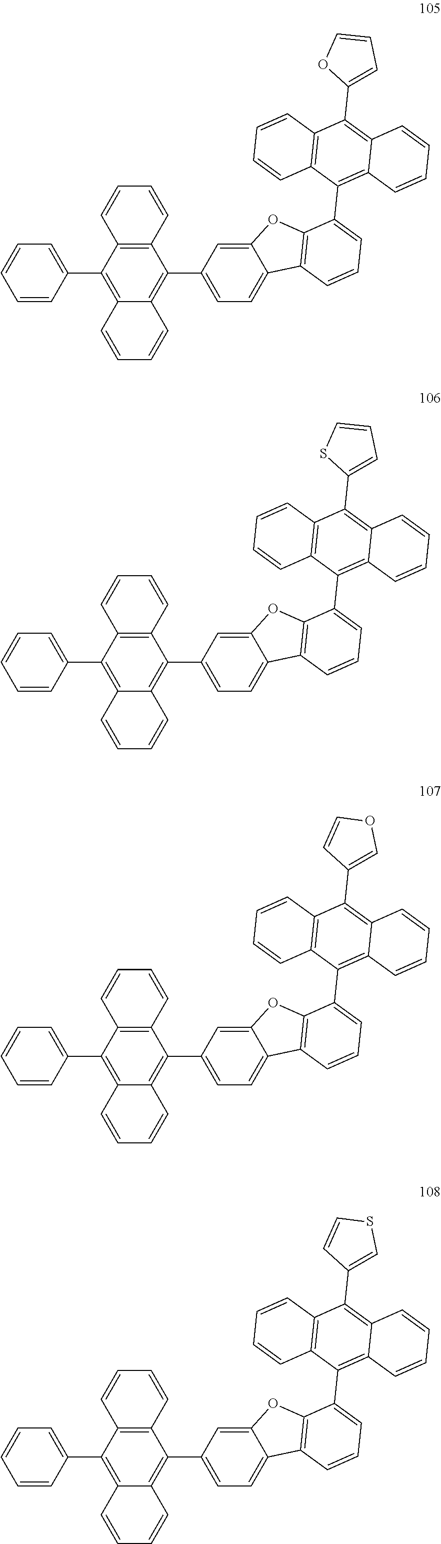 [SMILES: c1ccc(-c2c3ccccc3c(-c3ccc4c(c3)oc3c(-c5c6ccccc6c(-c6ccco6)c6ccccc56)cccc34)c3ccccc23)cc1 ; c1ccc(-c2c3ccccc3c(-c3ccc4c(c3)oc3c(-c5c6ccccc6c(-c6cccs6)c6ccccc56)cccc34)c3ccccc23)cc1 ; c1ccc(-c2c3ccccc3c(-c3ccc4c(c3)oc3c(-c5c6ccccc6c(-c6ccoc6)c6ccccc56)cccc34)c3ccccc23)cc1 ; c1ccc(-c2c3ccccc3c(-c3ccc4c(c3)oc3c(-c5c6ccccc6c(-c6ccsc6)c6ccccc56)cccc34)c3ccccc23)cc1]

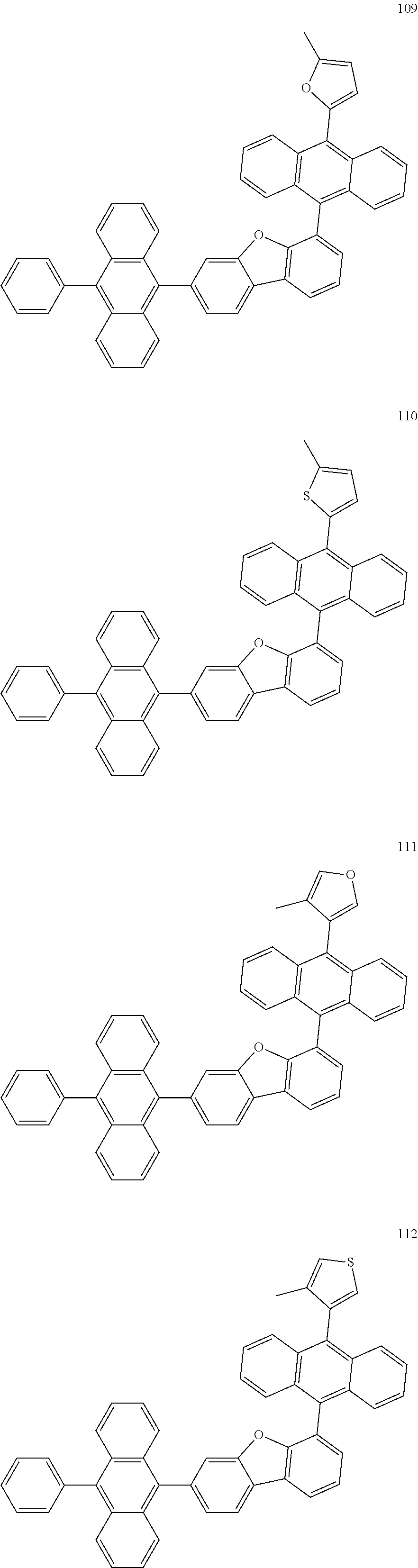 [SMILES: Cc1ccc(-c2c3ccccc3c(-c3cccc4c3oc3cc(-c5c6ccccc6c(-c6ccccc6)c6ccccc56)ccc34)c3ccccc23)o1 ; Cc1ccc(-c2c3ccccc3c(-c3cccc4c3oc3cc(-c5c6ccccc6c(-c6ccccc6)c6ccccc56)ccc34)c3ccccc23)s1 ; Cc1cocc1-c1c2ccccc2c(-c2cccc3c2oc2cc(-c4c5ccccc5c(-c5ccccc5)c5ccccc45)ccc23)c2ccccc12 ; Cc1cscc1-c1c2ccccc2c(-c2cccc3c2oc2cc(-c4c5ccccc5c(-c5ccccc5)c5ccccc45)ccc23)c2ccccc12]

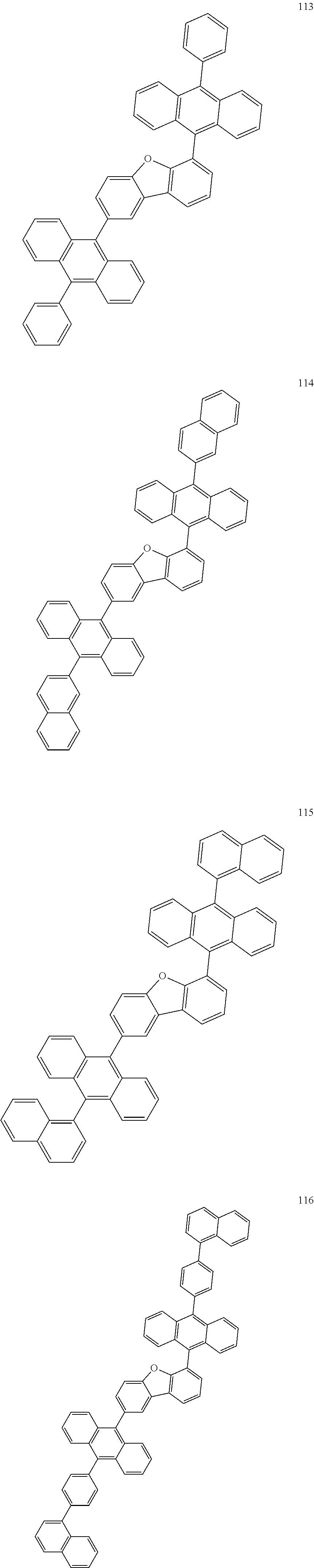 [SMILES: c1ccc(-c2c3ccccc3c(-c3ccc4oc5c(-c6c7ccccc7c(-c7ccccc7)c7ccccc67)cccc5c4c3)c3ccccc23)cc1 ; c1ccc2cc(-c3c4ccccc4c(-c4ccc5oc6c(-c7c8ccccc8c(-c8ccc9ccccc9c8)c8ccccc78)cccc6c5c4)c4ccccc34)ccc2c1 ; c1ccc2c(-c3c4ccccc4c(-c4ccc5oc6c(-c7c8ccccc8c(-c8cccc9ccccc89)c8ccccc78)cccc6c5c4)c4ccccc34)cccc2c1 ; c1ccc2c(-c3ccc(-c4c5ccccc5c(-c5ccc6oc7c(-c8c9ccccc9c(-c9ccc(-c%10cccc%11ccccc%10%11)cc9)c9ccccc89)cccc7c6c5)c5ccccc45)cc3)cccc2c1]

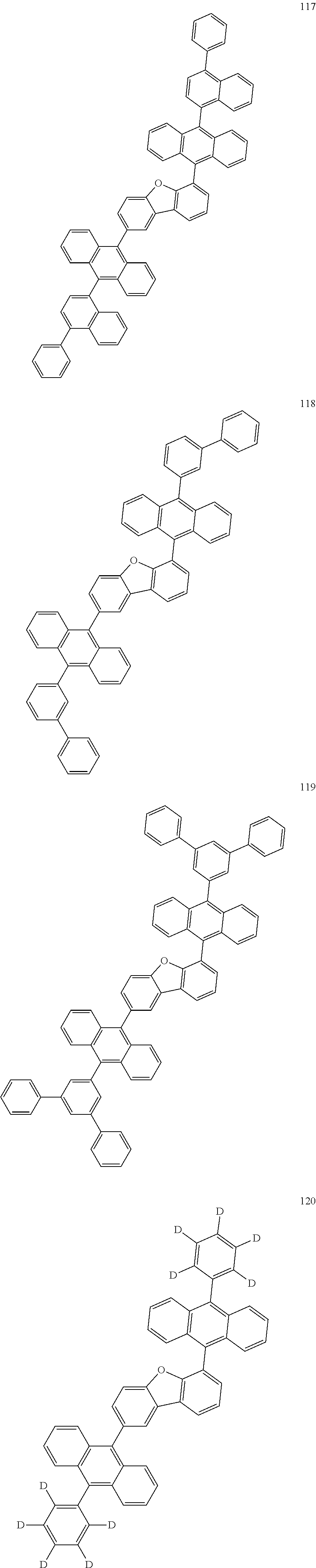

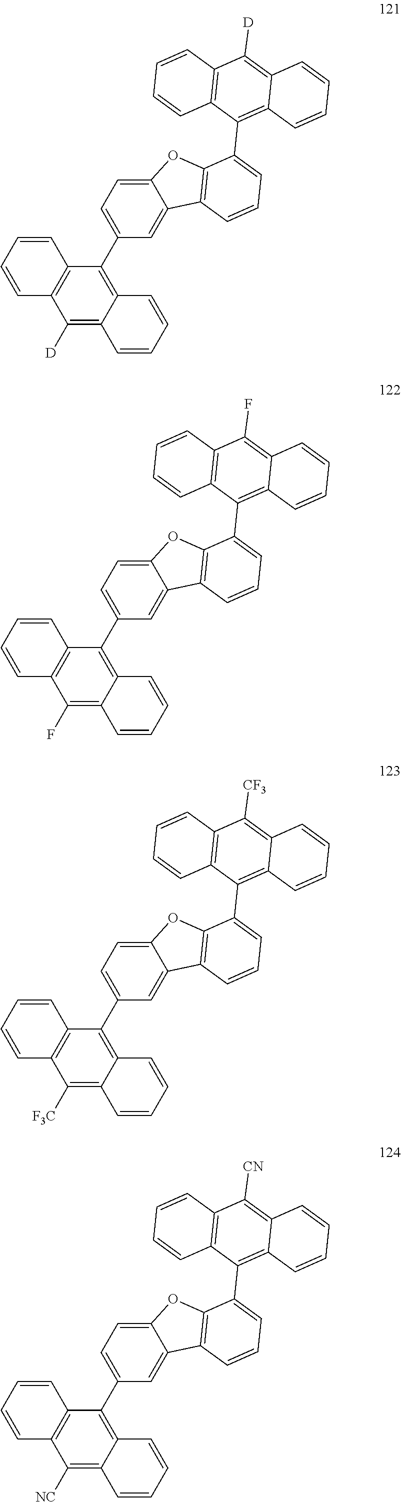 [SMILES: [2H]c1c2ccccc2c(-c2ccc3oc4c(-c5c6ccccc6c([2H])c6ccccc56)cccc4c3c2)c2ccccc12 ; Fc1c2ccccc2c(-c2ccc3oc4c(-c5c6ccccc6c(F)c6ccccc56)cccc4c3c2)c2ccccc12 ; FC(F)(F)c1c2ccccc2c(-c2ccc3oc4c(-c5c6ccccc6c(C(F)(F)F)c6ccccc56)cccc4c3c2)c2ccccc12 ; N#Cc1c2ccccc2c(-c2ccc3oc4c(-c5c6ccccc6c(C#N)c6ccccc56)cccc4c3c2)c2ccccc12]

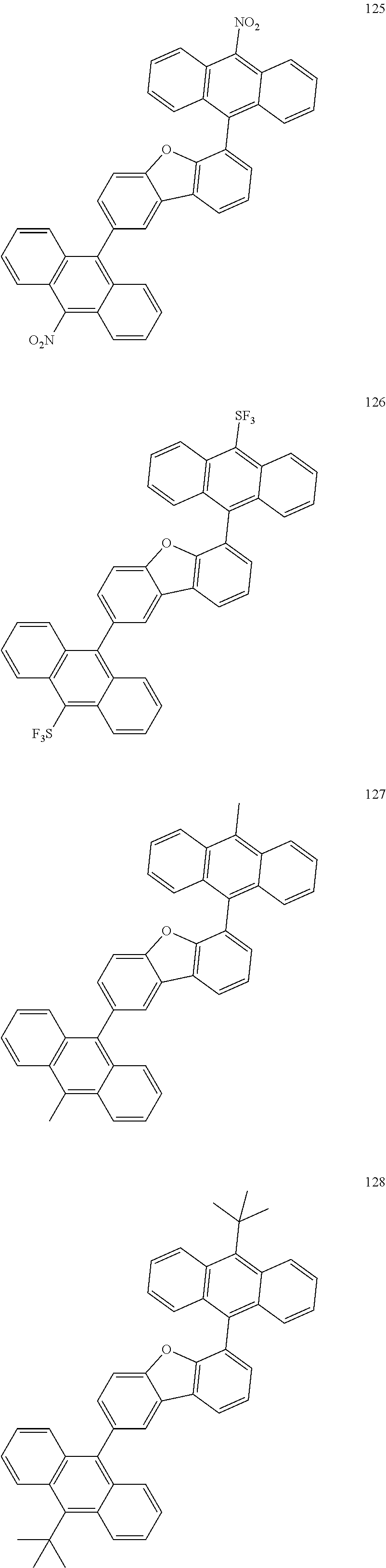

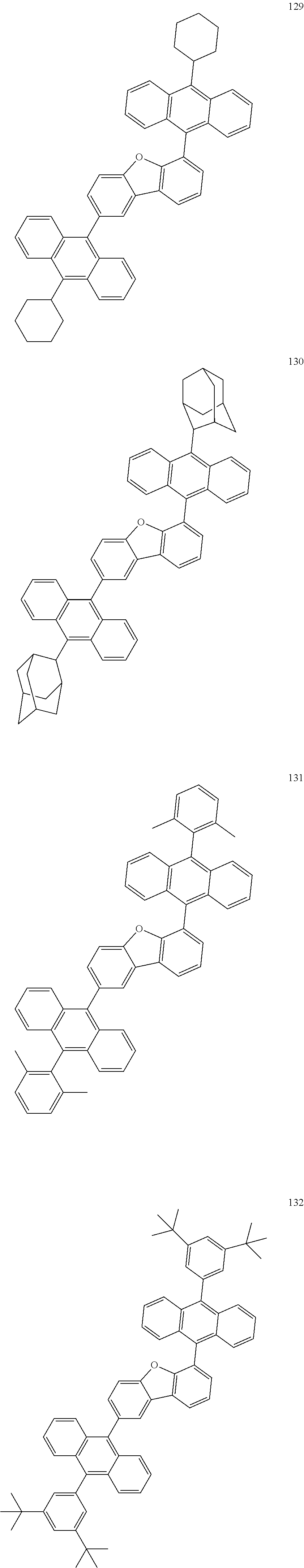 [SMILES: c1ccc2c(C3CCCCC3)c3ccccc3c(-c3ccc4oc5c(-c6c7ccccc7c(C7CCCCC7)c7ccccc67)cccc5c4c3)c2c1 ; c1ccc2c(C3C4CC5CC(C4)CC3C5)c3ccccc3c(-c3ccc4oc5c(-c6c7ccccc7c(C7C8CC9CC(C8)CC7C9)c7ccccc67)cccc5c4c3)c2c1 ; Cc1cccc(C)c1-c1c2ccccc2c(-c2ccc3oc4c(-c5c6ccccc6c(-c6c(C)cccc6C)c6ccccc56)cccc4c3c2)c2ccccc12 ; CC(C)(C)c1cc(-c2c3ccccc3c(-c3ccc4oc5c(-c6c7ccccc7c(-c7cc(C(C)(C)C)cc(C(C)(C)C)c7)c7ccccc67)cccc5c4c3)c3ccccc23)cc(C(C)(C)C)c1]

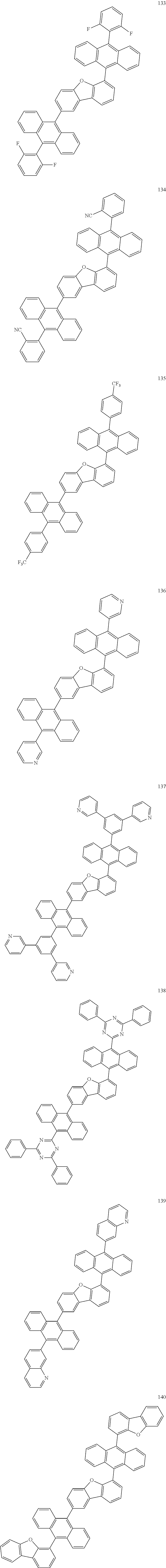

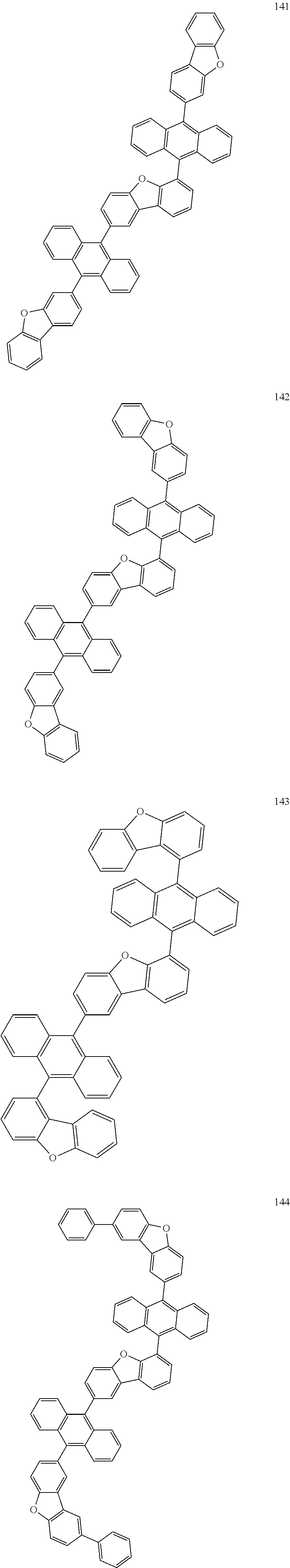

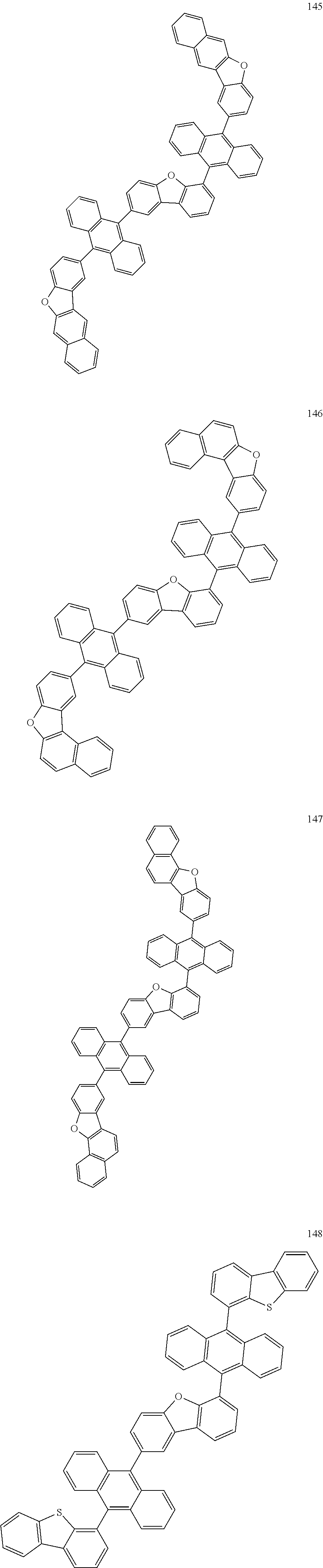

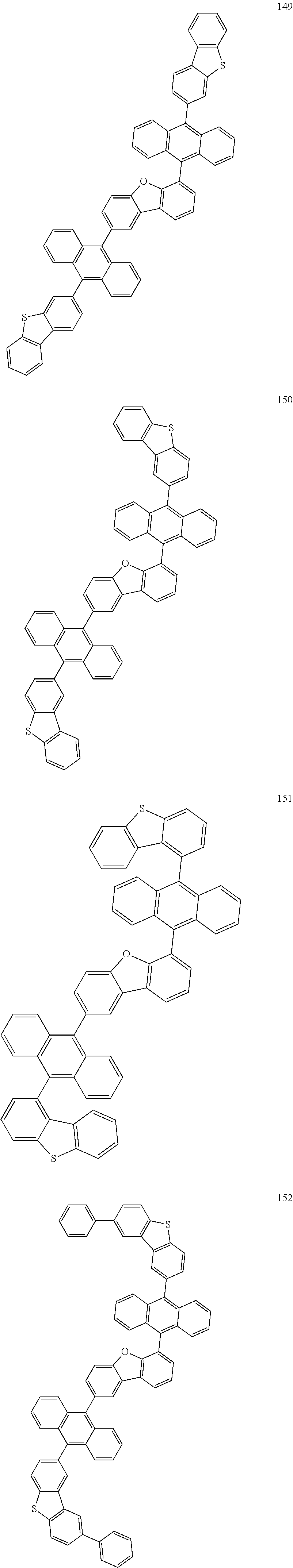

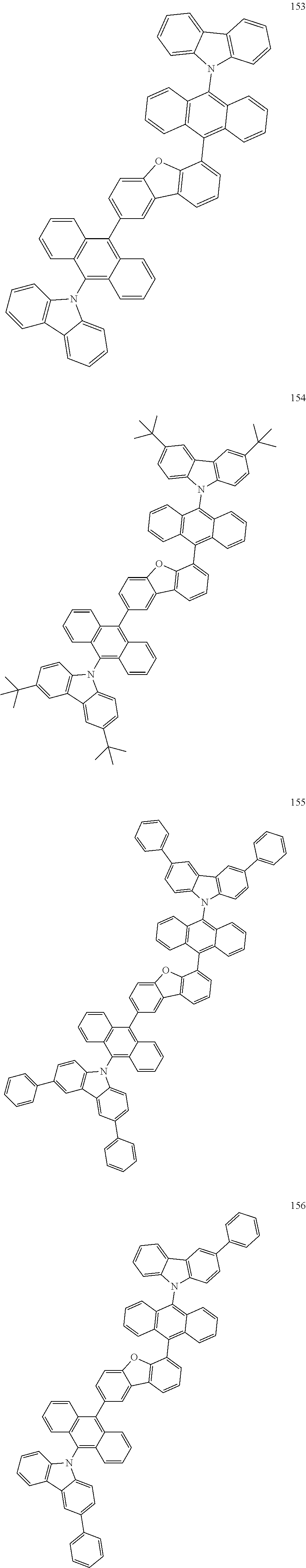

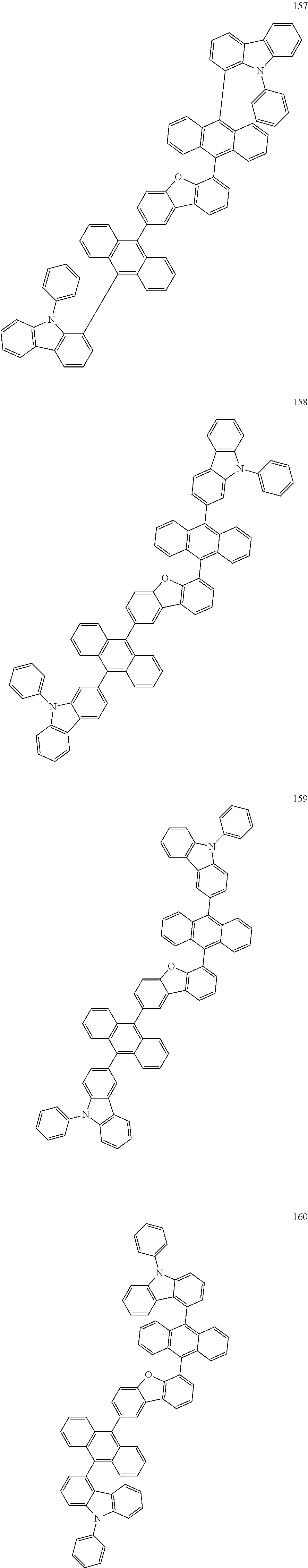

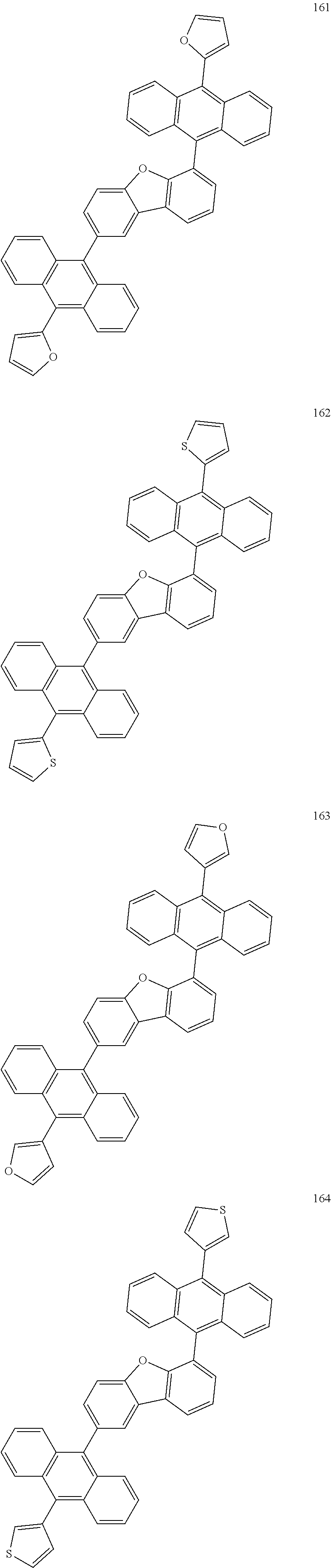 [SMILES: c1coc(-c2c3ccccc3c(-c3ccc4oc5c(-c6c7ccccc7c(-c7ccco7)c7ccccc67)cccc5c4c3)c3ccccc23)c1 ; c1csc(-c2c3ccccc3c(-c3ccc4oc5c(-c6c7ccccc7c(-c7cccs7)c7ccccc67)cccc5c4c3)c3ccccc23)c1 ; c1ccc2c(-c3ccc4oc5c(-c6c7ccccc7c(-c7ccoc7)c7ccccc67)cccc5c4c3)c3ccccc3c(-c3ccoc3)c2c1 ; c1ccc2c(-c3ccc4oc5c(-c6c7ccccc7c(-c7ccsc7)c7ccccc67)cccc5c4c3)c3ccccc3c(-c3ccsc3)c2c1]

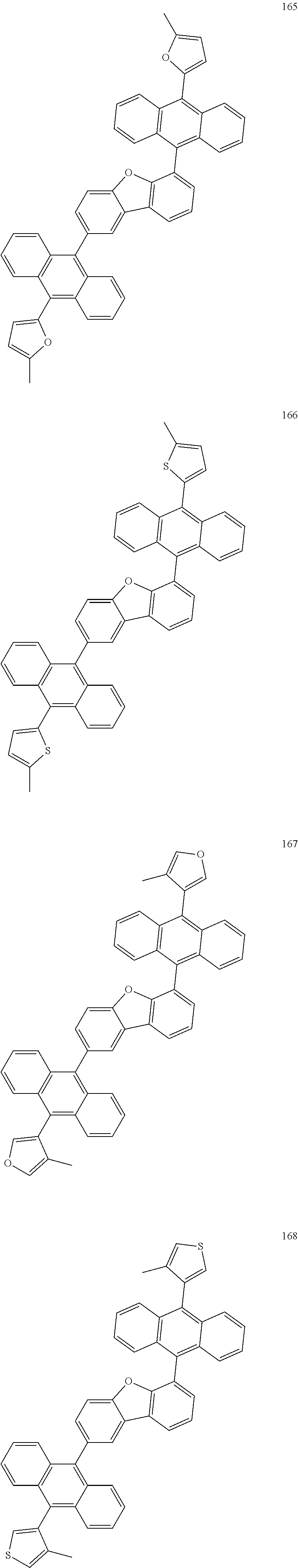 [SMILES: Cc1ccc(-c2c3ccccc3c(-c3ccc4oc5c(-c6c7ccccc7c(-c7ccc(C)o7)c7ccccc67)cccc5c4c3)c3ccccc23)o1 ; Cc1ccc(-c2c3ccccc3c(-c3ccc4oc5c(-c6c7ccccc7c(-c7ccc(C)s7)c7ccccc67)cccc5c4c3)c3ccccc23)s1 ; Cc1cocc1-c1c2ccccc2c(-c2ccc3oc4c(-c5c6ccccc6c(-c6cocc6C)c6ccccc56)cccc4c3c2)c2ccccc12 ; Cc1cscc1-c1c2ccccc2c(-c2ccc3oc4c(-c5c6ccccc6c(-c6cscc6C)c6ccccc56)cccc4c3c2)c2ccccc12]

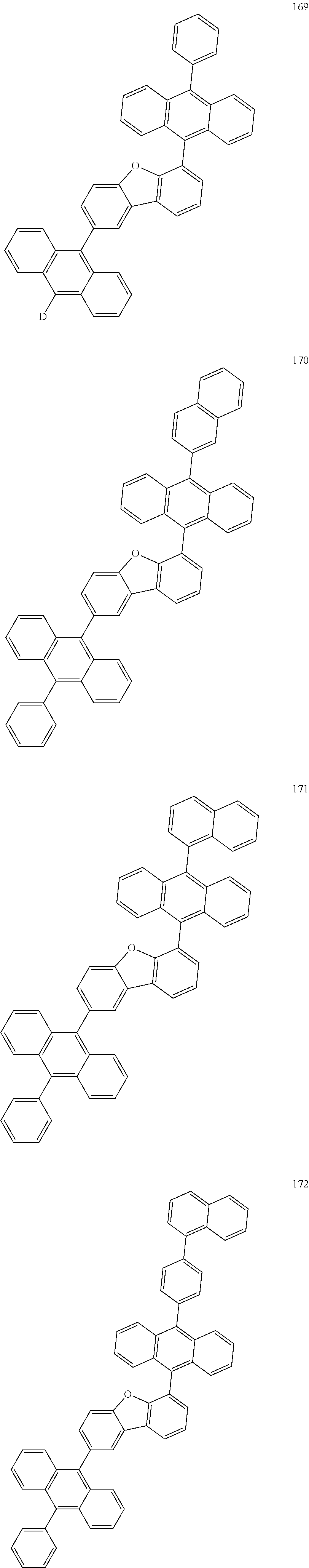 [SMILES: [2H]c1c2ccccc2c(-c2ccc3oc4c(-c5c6ccccc6c(-c6ccccc6)c6ccccc56)cccc4c3c2)c2ccccc12 ; c1ccc(-c2c3ccccc3c(-c3ccc4oc5c(-c6c7ccccc7c(-c7ccc8ccccc8c7)c7ccccc67)cccc5c4c3)c3ccccc23)cc1 ; c1ccc(-c2c3ccccc3c(-c3ccc4oc5c(-c6c7ccccc7c(-c7cccc8ccccc78)c7ccccc67)cccc5c4c3)c3ccccc23)cc1 ; c1ccc(-c2c3ccccc3c(-c3ccc4oc5c(-c6c7ccccc7c(-c7ccc(-c8cccc9ccccc89)cc7)c7ccccc67)cccc5c4c3)c3ccccc23)cc1]

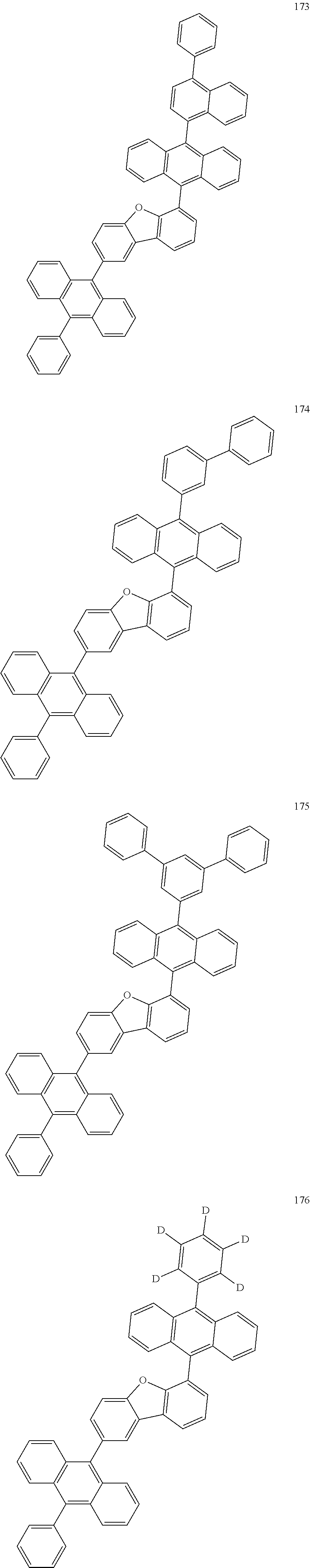 [SMILES: c1ccc(-c2ccc(-c3c4ccccc4c(-c4cccc5c4oc4ccc(-c6c7ccccc7c(-c7ccccc7)c7ccccc67)cc45)c4ccccc34)c3ccccc23)cc1 ; c1ccc(-c2cccc(-c3c4ccccc4c(-c4cccc5c4oc4ccc(-c6c7ccccc7c(-c7ccccc7)c7ccccc67)cc45)c4ccccc34)c2)cc1 ; c1ccc(-c2cc(-c3ccccc3)cc(-c3c4ccccc4c(-c4cccc5c4oc4ccc(-c6c7ccccc7c(-c7ccccc7)c7ccccc67)cc45)c4ccccc34)c2)cc1 ; [2H]c1c([2H])c([2H])c(-c2c3ccccc3c(-c3cccc4c3oc3ccc(-c5c6ccccc6c(-c6ccccc6)c6ccccc56)cc34)c3ccccc23)c([2H])c1[2H]]

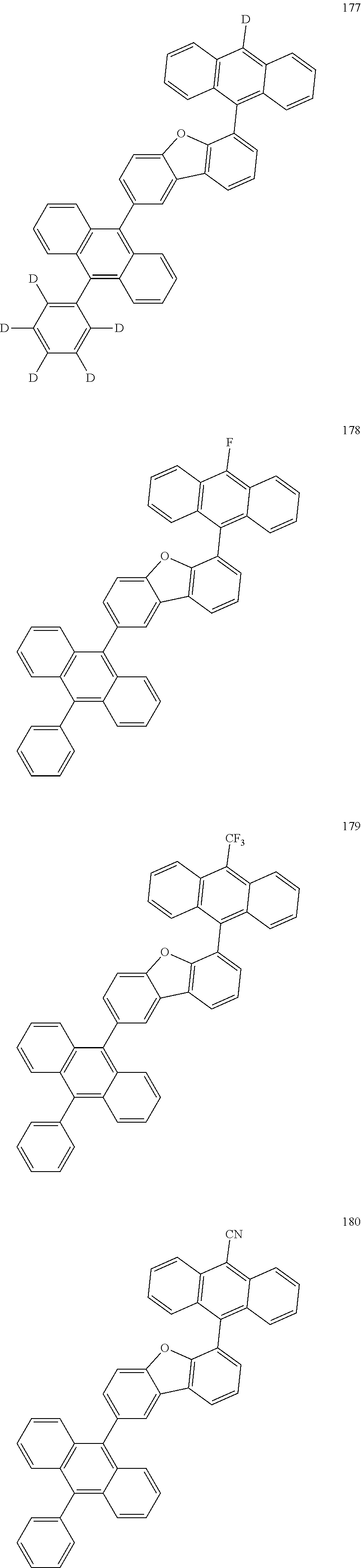 [SMILES: [2H]c1c([2H])c([2H])c(-c2c3ccccc3c(-c3ccc4oc5c(-c6c7ccccc7c([2H])c7ccccc67)cccc5c4c3)c3ccccc23)c([2H])c1[2H] ; Fc1c2ccccc2c(-c2cccc3c2oc2ccc(-c4c5ccccc5c(-c5ccccc5)c5ccccc45)cc23)c2ccccc12 ; FC(F)(F)c1c2ccccc2c(-c2cccc3c2oc2ccc(-c4c5ccccc5c(-c5ccccc5)c5ccccc45)cc23)c2ccccc12 ; N#Cc1c2ccccc2c(-c2cccc3c2oc2ccc(-c4c5ccccc5c(-c5ccccc5)c5ccccc45)cc23)c2ccccc12]

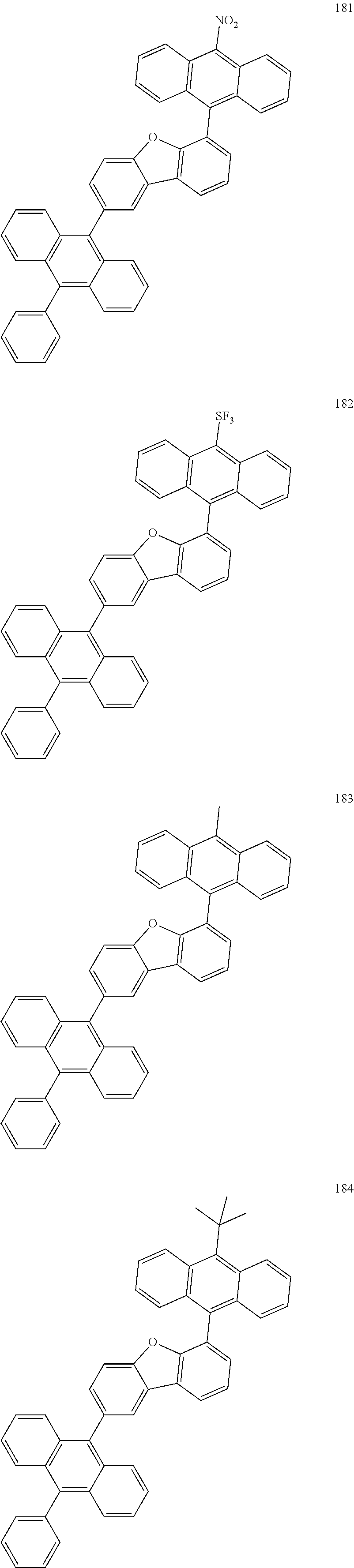

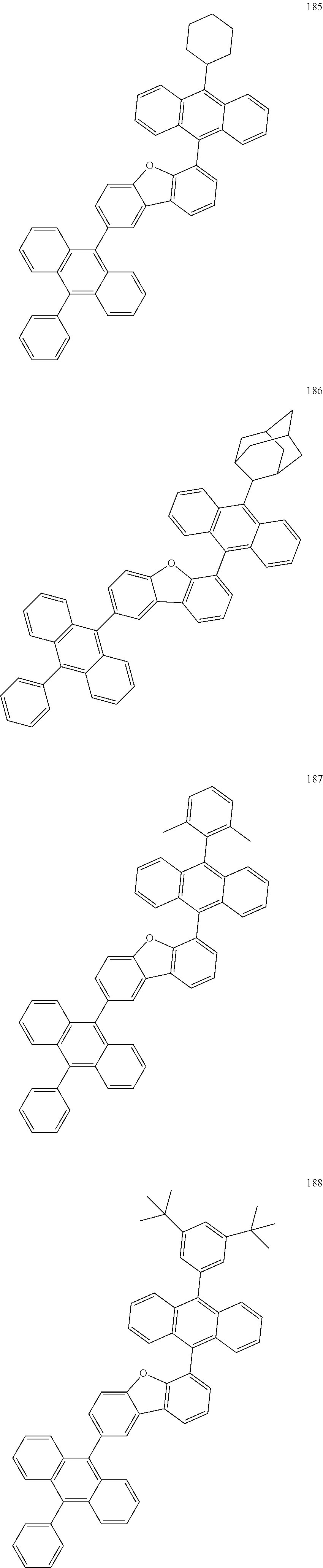 [SMILES: c1ccc(-c2c3ccccc3c(-c3ccc4oc5c(-c6c7ccccc7c(C7CCCCC7)c7ccccc67)cccc5c4c3)c3ccccc23)cc1 ; c1ccc(-c2c3ccccc3c(-c3ccc4oc5c(-c6c7ccccc7c(C7C8CC9CC(C8)CC7C9)c7ccccc67)cccc5c4c3)c3ccccc23)cc1 ; Cc1cccc(C)c1-c1c2ccccc2c(-c2cccc3c2oc2ccc(-c4c5ccccc5c(-c5ccccc5)c5ccccc45)cc23)c2ccccc12 ; CC(C)(C)c1cc(-c2c3ccccc3c(-c3cccc4c3oc3ccc(-c5c6ccccc6c(-c6ccccc6)c6ccccc56)cc34)c3ccccc23)cc(C(C)(C)C)c1]

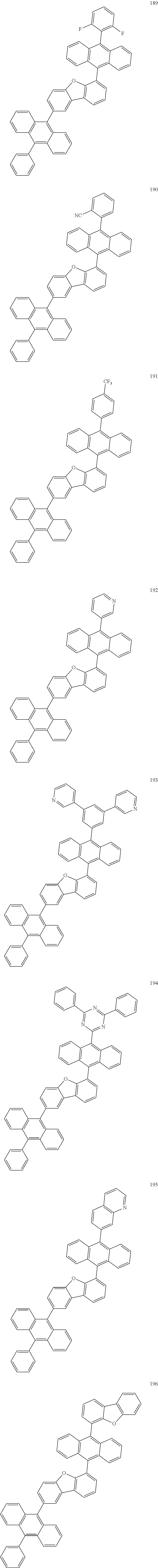

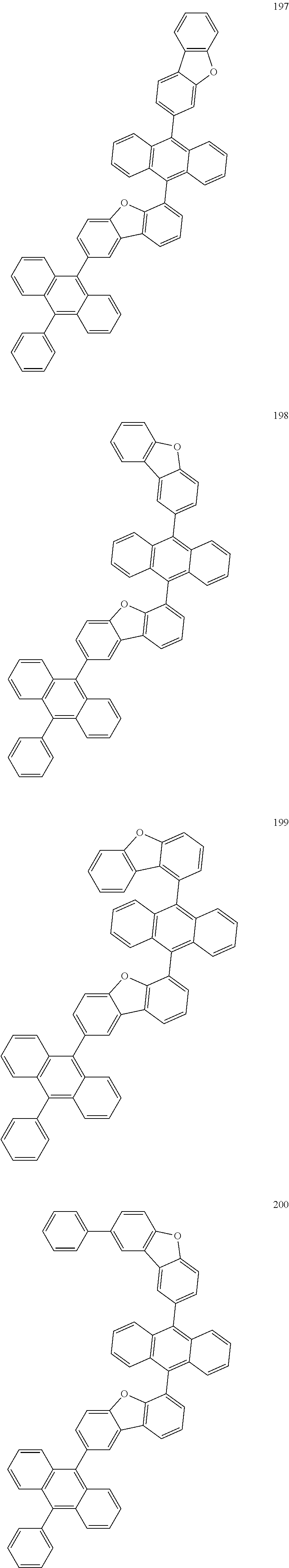 [SMILES: c1ccc(-c2c3ccccc3c(-c3ccc4oc5c(-c6c7ccccc7c(-c7ccc8c(c7)oc7ccccc78)c7ccccc67)cccc5c4c3)c3ccccc23)cc1 ; c1ccc(-c2c3ccccc3c(-c3ccc4oc5c(-c6c7ccccc7c(-c7ccc8oc9ccccc9c8c7)c7ccccc67)cccc5c4c3)c3ccccc23)cc1 ; c1ccc(-c2c3ccccc3c(-c3ccc4oc5c(-c6c7ccccc7c(-c7cccc8oc9ccccc9c78)c7ccccc67)cccc5c4c3)c3ccccc23)cc1 ; c1ccc(-c2ccc3oc4ccc(-c5c6ccccc6c(-c6cccc7c6oc6ccc(-c8c9ccccc9c(-c9ccccc9)c9ccccc89)cc67)c6ccccc56)cc4c3c2)cc1]

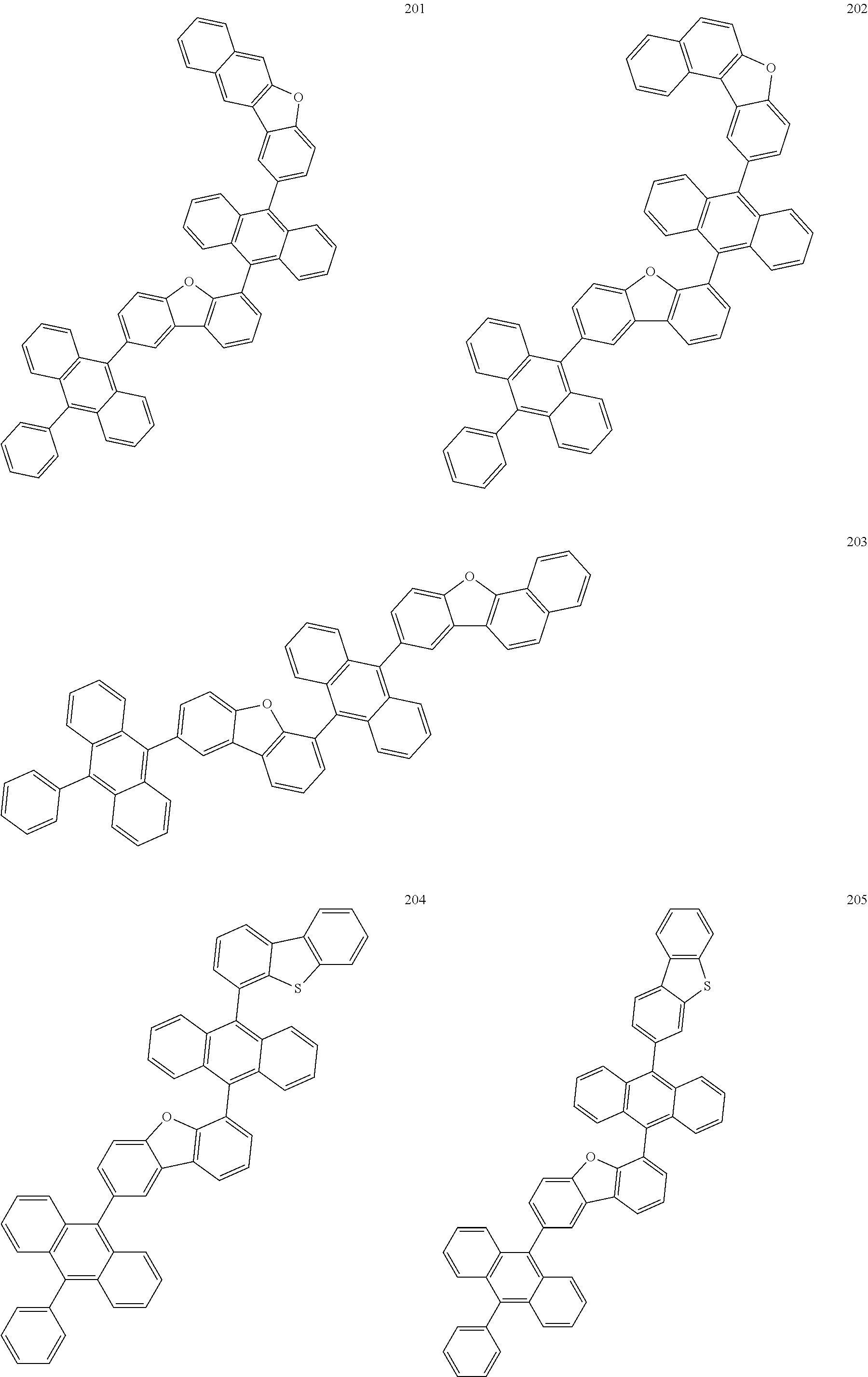

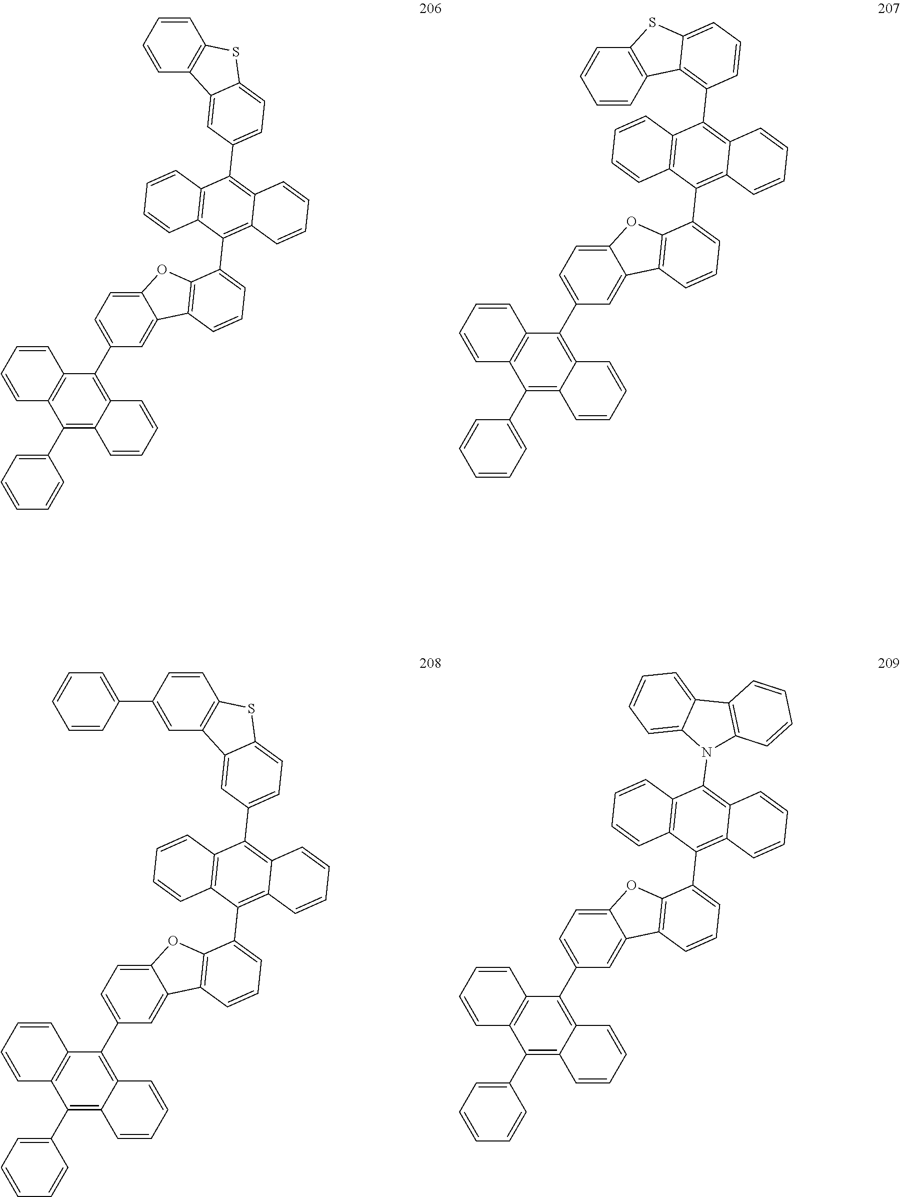 [SMILES: c1ccc(-c2c3ccccc3c(-c3ccc4oc5c(-c6c7ccccc7c(-c7ccc8sc9ccccc9c8c7)c7ccccc67)cccc5c4c3)c3ccccc23)cc1 ; c1ccc(-c2c3ccccc3c(-c3ccc4oc5c(-c6c7ccccc7c(-c7cccc8sc9ccccc9c78)c7ccccc67)cccc5c4c3)c3ccccc23)cc1 ; c1ccc(-c2ccc3sc4ccc(-c5c6ccccc6c(-c6cccc7c6oc6ccc(-c8c9ccccc9c(-c9ccccc9)c9ccccc89)cc67)c6ccccc56)cc4c3c2)cc1 ; c1ccc(-c2c3ccccc3c(-c3ccc4oc5c(-c6c7ccccc7c(-n7c8ccccc8c8ccccc87)c7ccccc67)cccc5c4c3)c3ccccc23)cc1]

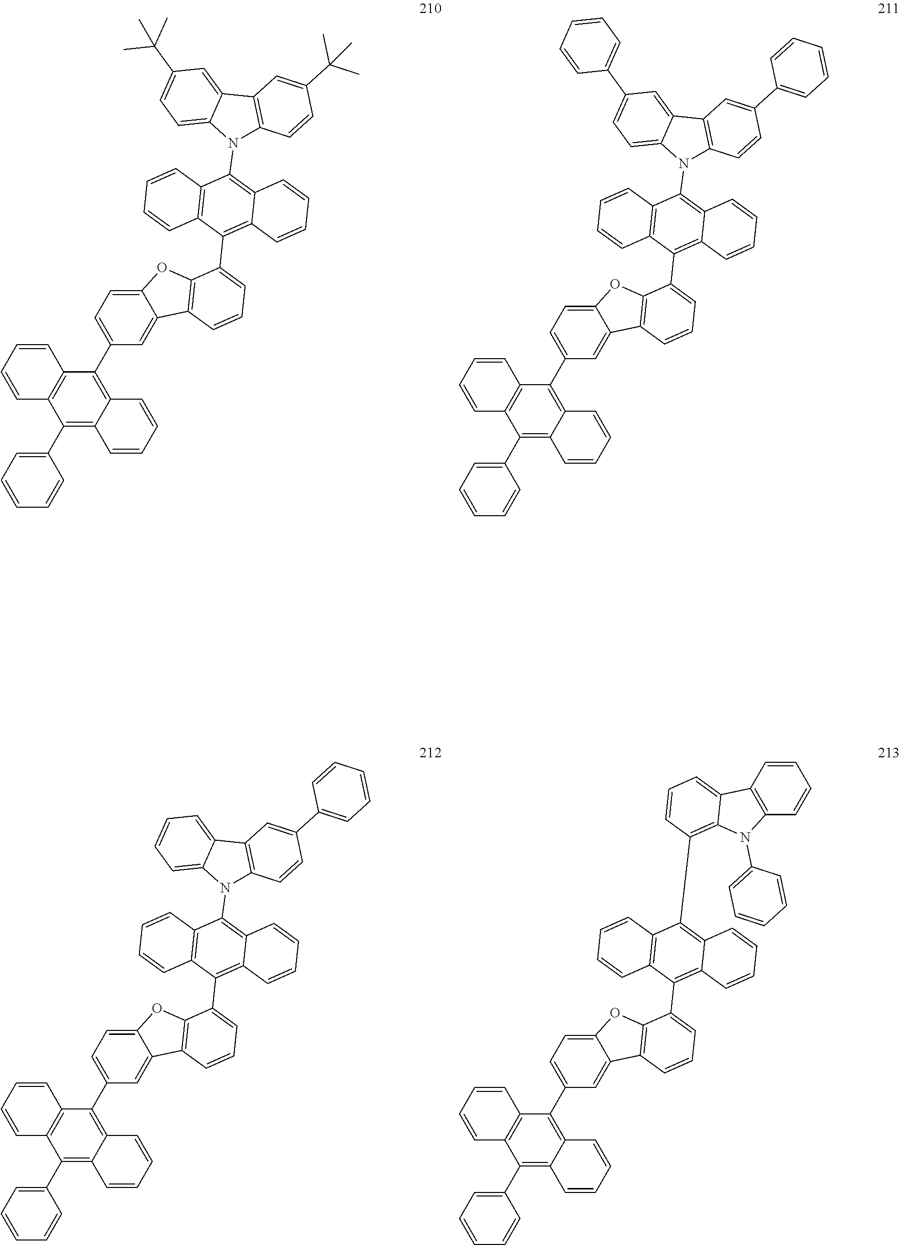 [SMILES: CC(C)(C)c1ccc2c(c1)c1cc(C(C)(C)C)ccc1n2-c1c2ccccc2c(-c2cccc3c2oc2ccc(-c4c5ccccc5c(-c5ccccc5)c5ccccc45)cc23)c2ccccc12 ; c1ccc(-c2ccc3c(c2)c2cc(-c4ccccc4)ccc2n3-c2c3ccccc3c(-c3cccc4c3oc3ccc(-c5c6ccccc6c(-c6ccccc6)c6ccccc56)cc34)c3ccccc23)cc1 ; c1ccc(-c2ccc3c(c2)c2ccccc2n3-c2c3ccccc3c(-c3cccc4c3oc3ccc(-c5c6ccccc6c(-c6ccccc6)c6ccccc56)cc34)c3ccccc23)cc1 ; c1ccc(-c2c3ccccc3c(-c3ccc4oc5c(-c6c7ccccc7c(-c7cccc8c9ccccc9n(-c9ccccc9)c78)c7ccccc67)cccc5c4c3)c3ccccc23)cc1]

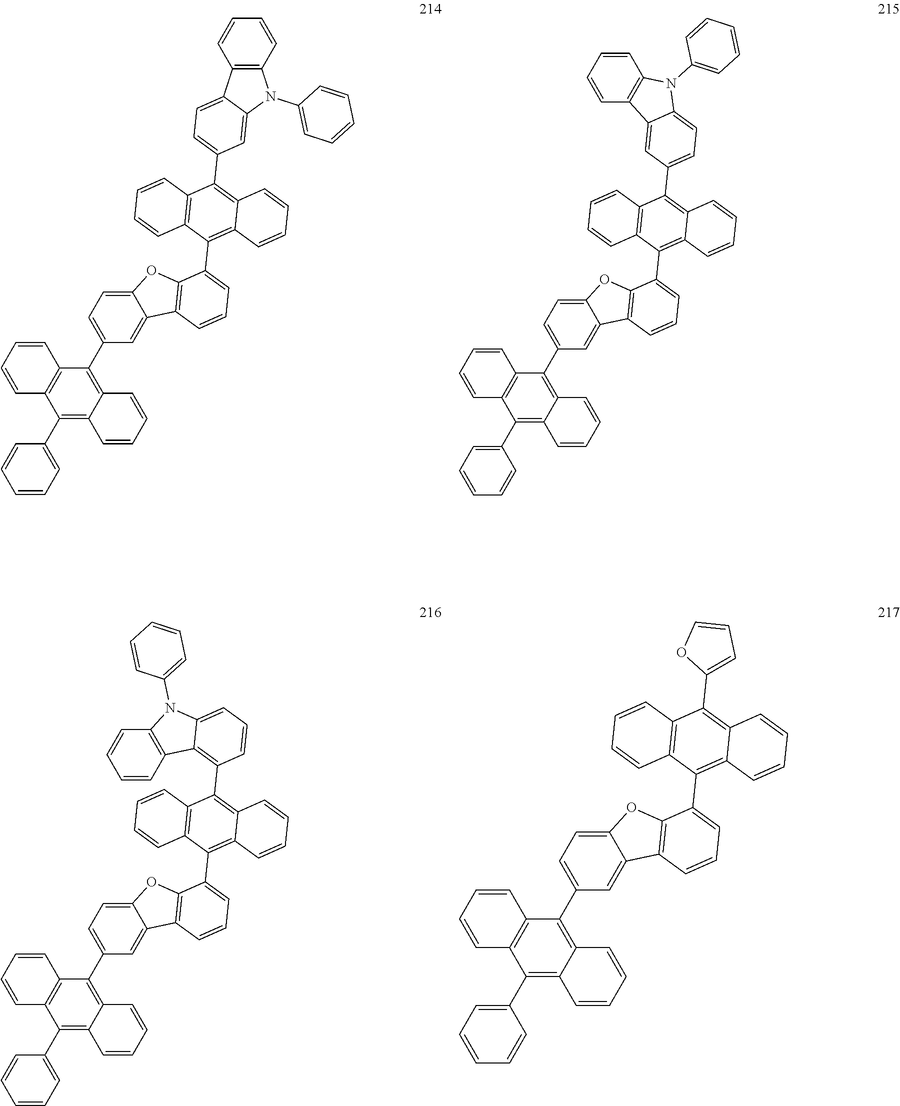 [SMILES: c1ccc(-c2c3ccccc3c(-c3ccc4oc5c(-c6c7ccccc7c(-c7ccc8c9ccccc9n(-c9ccccc9)c8c7)c7ccccc67)cccc5c4c3)c3ccccc23)cc1 ; c1ccc(-c2c3ccccc3c(-c3ccc4oc5c(-c6c7ccccc7c(-c7ccc8c(c7)c7ccccc7n8-c7ccccc7)c7ccccc67)cccc5c4c3)c3ccccc23)cc1 ; c1ccc(-c2c3ccccc3c(-c3ccc4oc5c(-c6c7ccccc7c(-c7cccc8c7c7ccccc7n8-c7ccccc7)c7ccccc67)cccc5c4c3)c3ccccc23)cc1 ; c1ccc(-c2c3ccccc3c(-c3ccc4oc5c(-c6c7ccccc7c(-c7ccco7)c7ccccc67)cccc5c4c3)c3ccccc23)cc1]

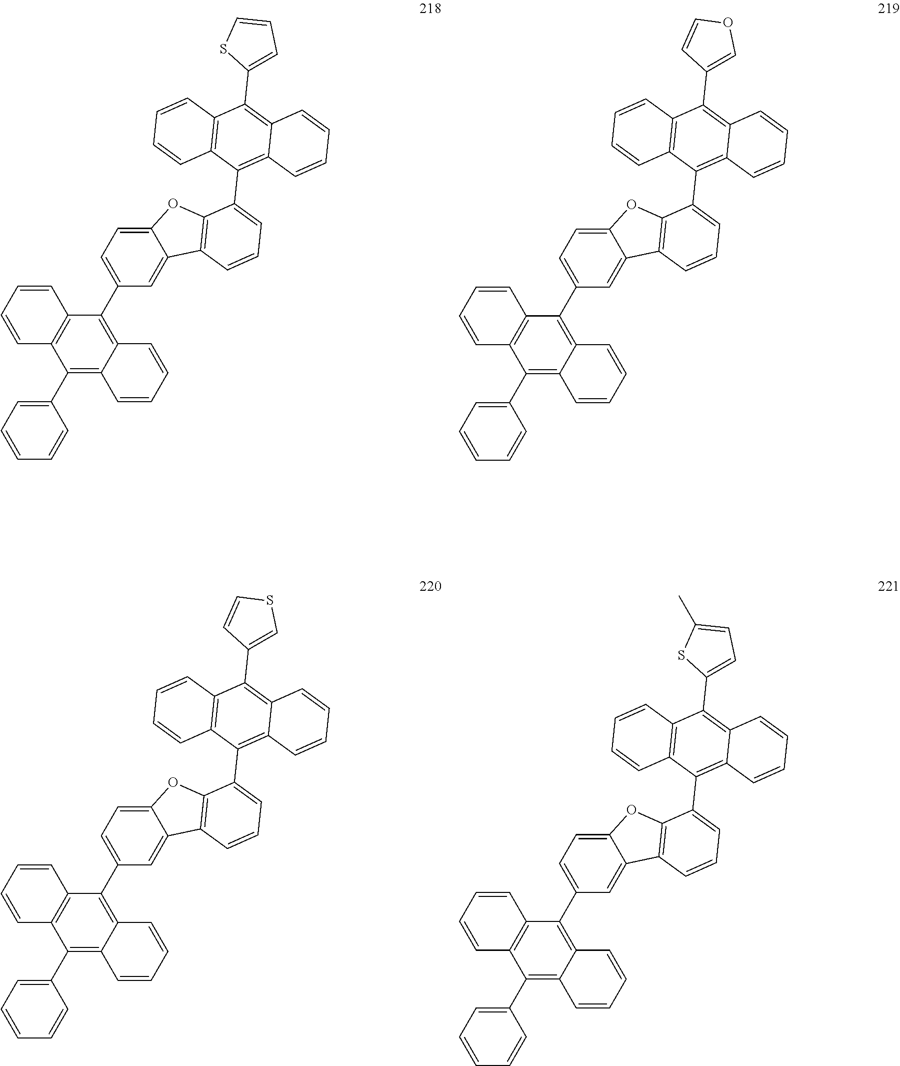 [SMILES: c1ccc(-c2c3ccccc3c(-c3ccc4oc5c(-c6c7ccccc7c(-c7cccs7)c7ccccc67)cccc5c4c3)c3ccccc23)cc1 ; c1ccc(-c2c3ccccc3c(-c3ccc4oc5c(-c6c7ccccc7c(-c7ccoc7)c7ccccc67)cccc5c4c3)c3ccccc23)cc1 ; c1ccc(-c2c3ccccc3c(-c3ccc4oc5c(-c6c7ccccc7c(-c7ccsc7)c7ccccc67)cccc5c4c3)c3ccccc23)cc1 ; Cc1ccc(-c2c3ccccc3c(-c3cccc4c3oc3ccc(-c5c6ccccc6c(-c6ccccc6)c6ccccc56)cc34)c3ccccc23)s1]

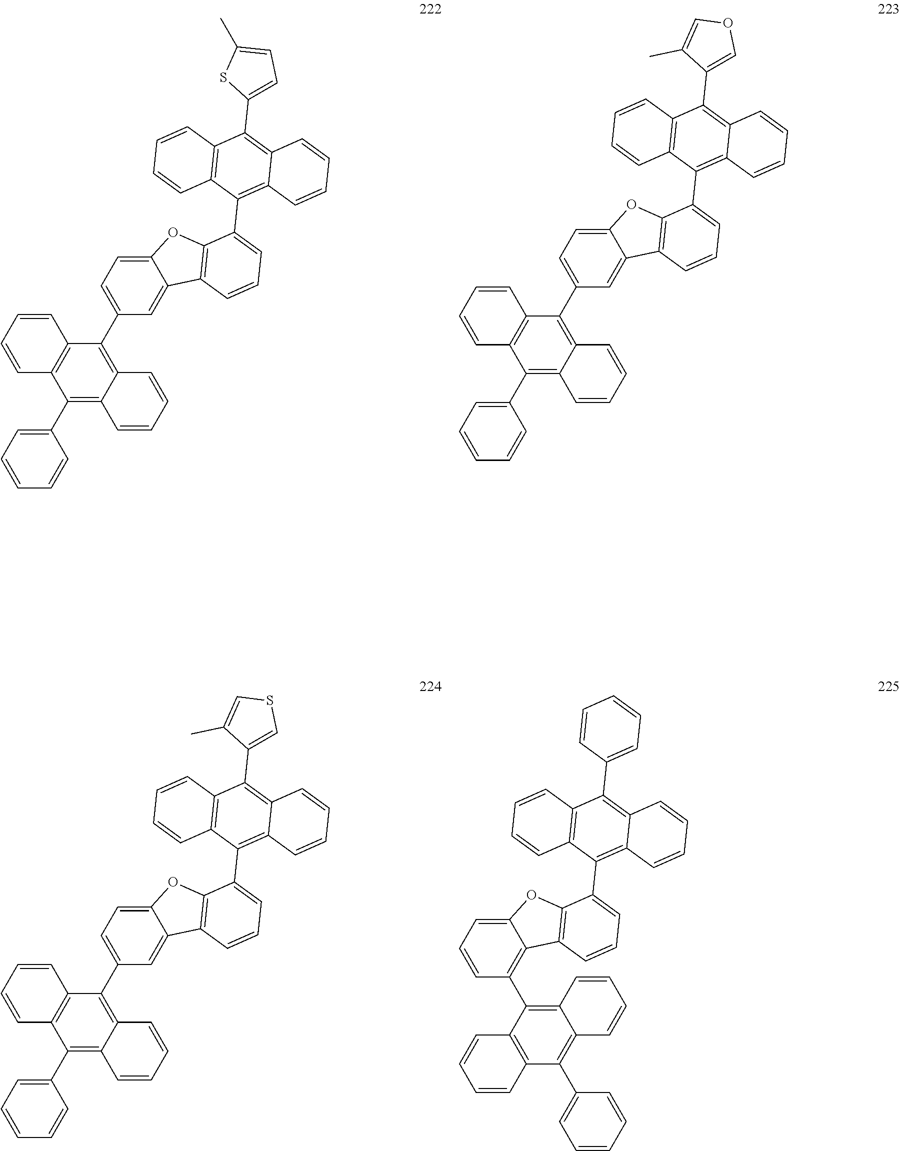 [SMILES: Cc1ccc(-c2c3ccccc3c(-c3cccc4c3oc3ccc(-c5c6ccccc6c(-c6ccccc6)c6ccccc56)cc34)c3ccccc23)s1 ; Cc1cocc1-c1c2ccccc2c(-c2cccc3c2oc2ccc(-c4c5ccccc5c(-c5ccccc5)c5ccccc45)cc23)c2ccccc12 ; Cc1cscc1-c1c2ccccc2c(-c2cccc3c2oc2ccc(-c4c5ccccc5c(-c5ccccc5)c5ccccc45)cc23)c2ccccc12 ; c1ccc(-c2c3ccccc3c(-c3cccc4c3oc3cccc(-c5c6ccccc6c(-c6ccccc6)c6ccccc56)c34)c3ccccc23)cc1]

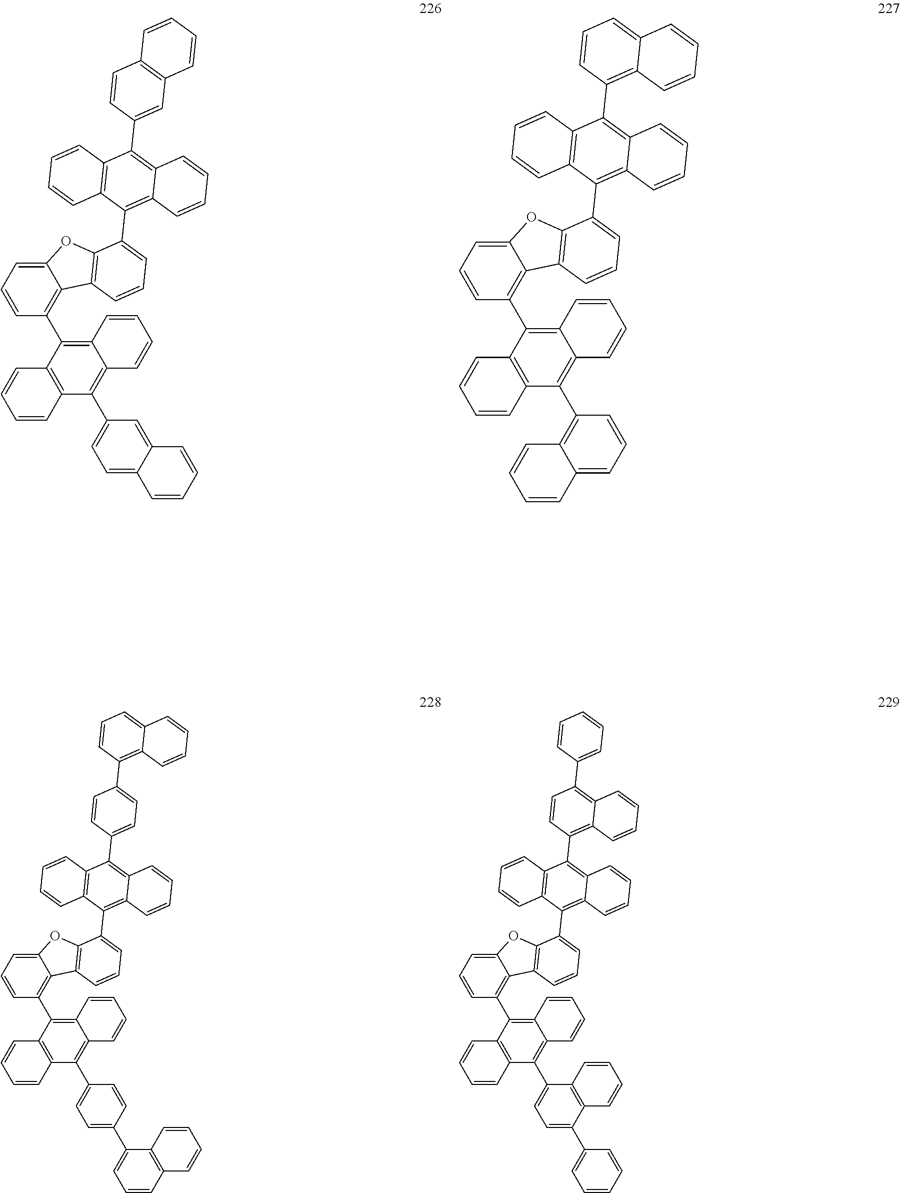 [SMILES: c1ccc2cc(-c3c4ccccc4c(-c4cccc5c4oc4cccc(-c6c7ccccc7c(-c7ccc8ccccc8c7)c7ccccc67)c45)c4ccccc34)ccc2c1 ; c1ccc2c(-c3c4ccccc4c(-c4cccc5c4oc4cccc(-c6c7ccccc7c(-c7cccc8ccccc78)c7ccccc67)c45)c4ccccc34)cccc2c1 ; c1ccc2c(-c3ccc(-c4c5ccccc5c(-c5cccc6c5oc5cccc(-c7c8ccccc8c(-c8ccc(-c9cccc%10ccccc9%10)cc8)c8ccccc78)c56)c5ccccc45)cc3)cccc2c1 ; c1ccc(-c2ccc(-c3c4ccccc4c(-c4cccc5c4oc4cccc(-c6c7ccccc7c(-c7ccc(-c8ccccc8)c8ccccc78)c7ccccc67)c45)c4ccccc34)c3ccccc23)cc1]

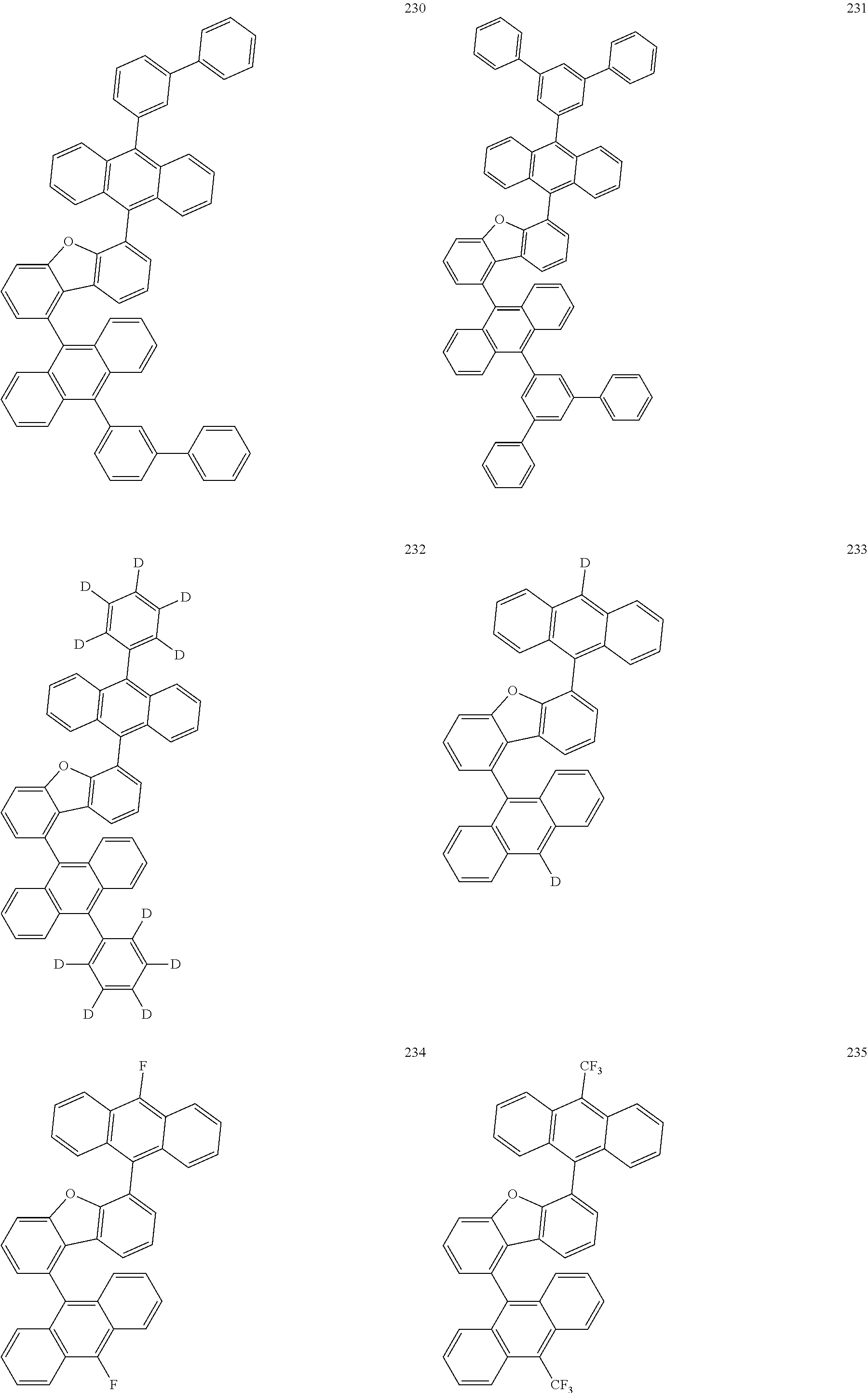

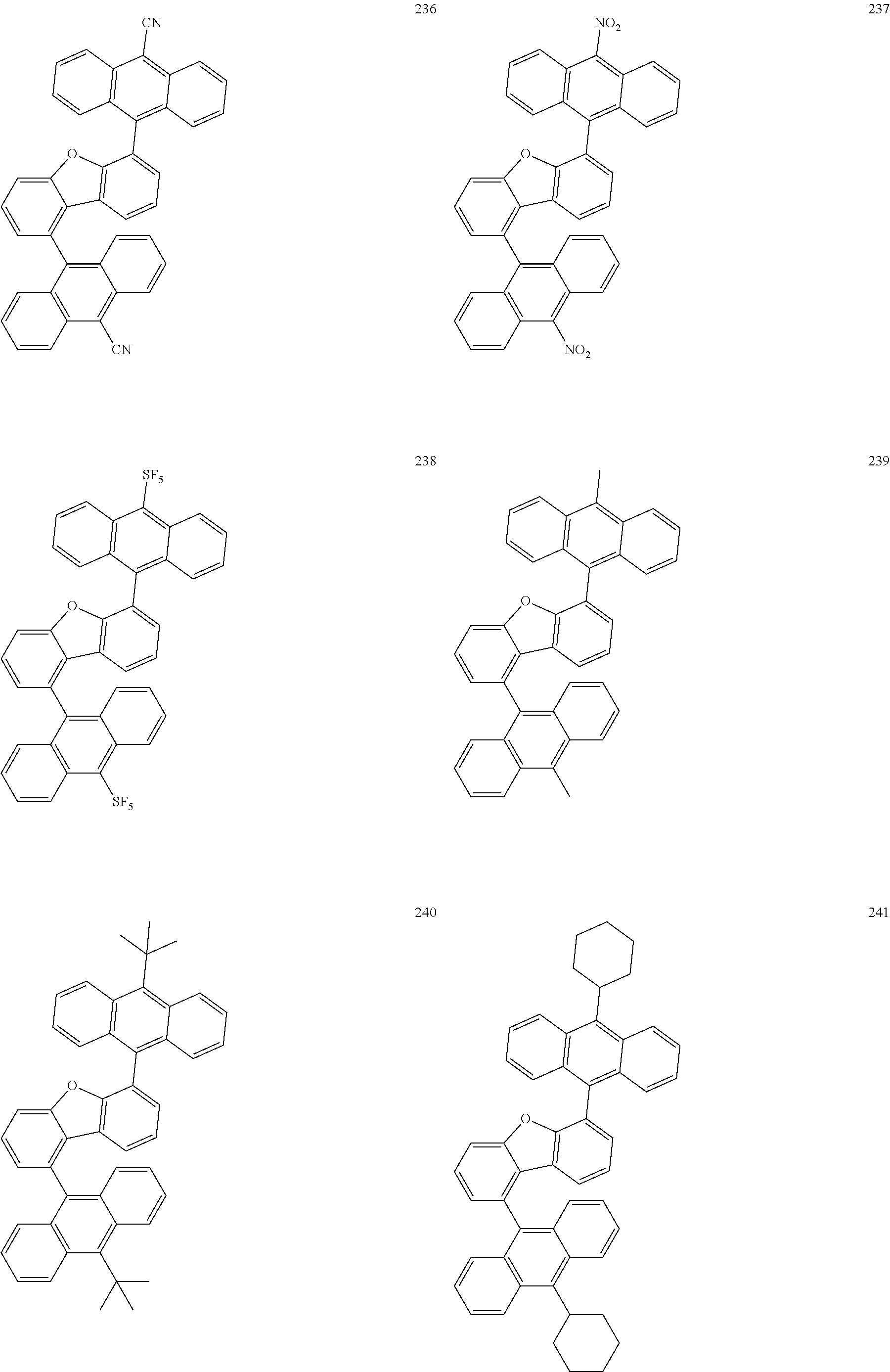

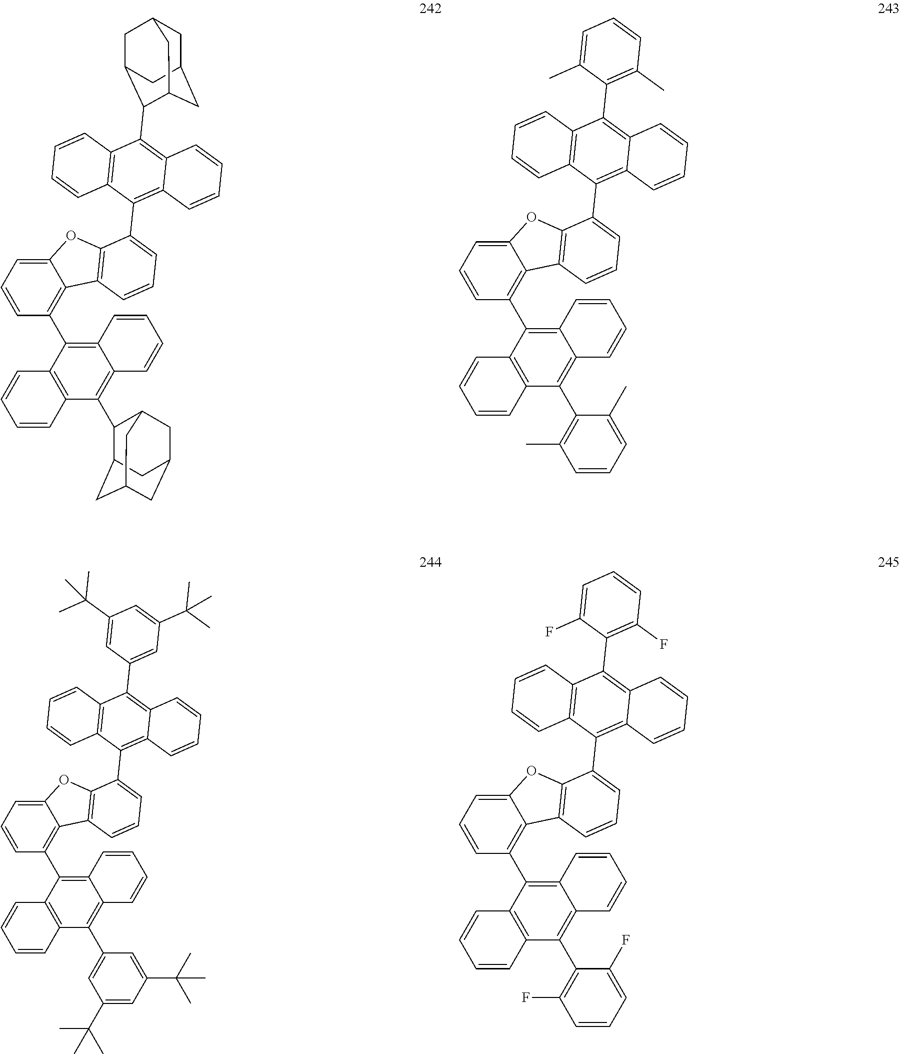 [SMILES: c1cc(-c2c3ccccc3c(C3C4CC5CC(C4)CC3C5)c3ccccc23)c2c(c1)oc1c(-c3c4ccccc4c(C4C5CC6CC(C5)CC4C6)c4ccccc34)cccc12 ; Cc1cccc(C)c1-c1c2ccccc2c(-c2cccc3c2oc2cccc(-c4c5ccccc5c(-c5c(C)cccc5C)c5ccccc45)c23)c2ccccc12 ; CC(C)(C)c1cc(-c2c3ccccc3c(-c3cccc4c3oc3cccc(-c5c6ccccc6c(-c6cc(C(C)(C)C)cc(C(C)(C)C)c6)c6ccccc56)c34)c3ccccc23)cc(C(C)(C)C)c1 ; Fc1cccc(F)c1-c1c2ccccc2c(-c2cccc3c2oc2cccc(-c4c5ccccc5c(-c5c(F)cccc5F)c5ccccc45)c23)c2ccccc12]

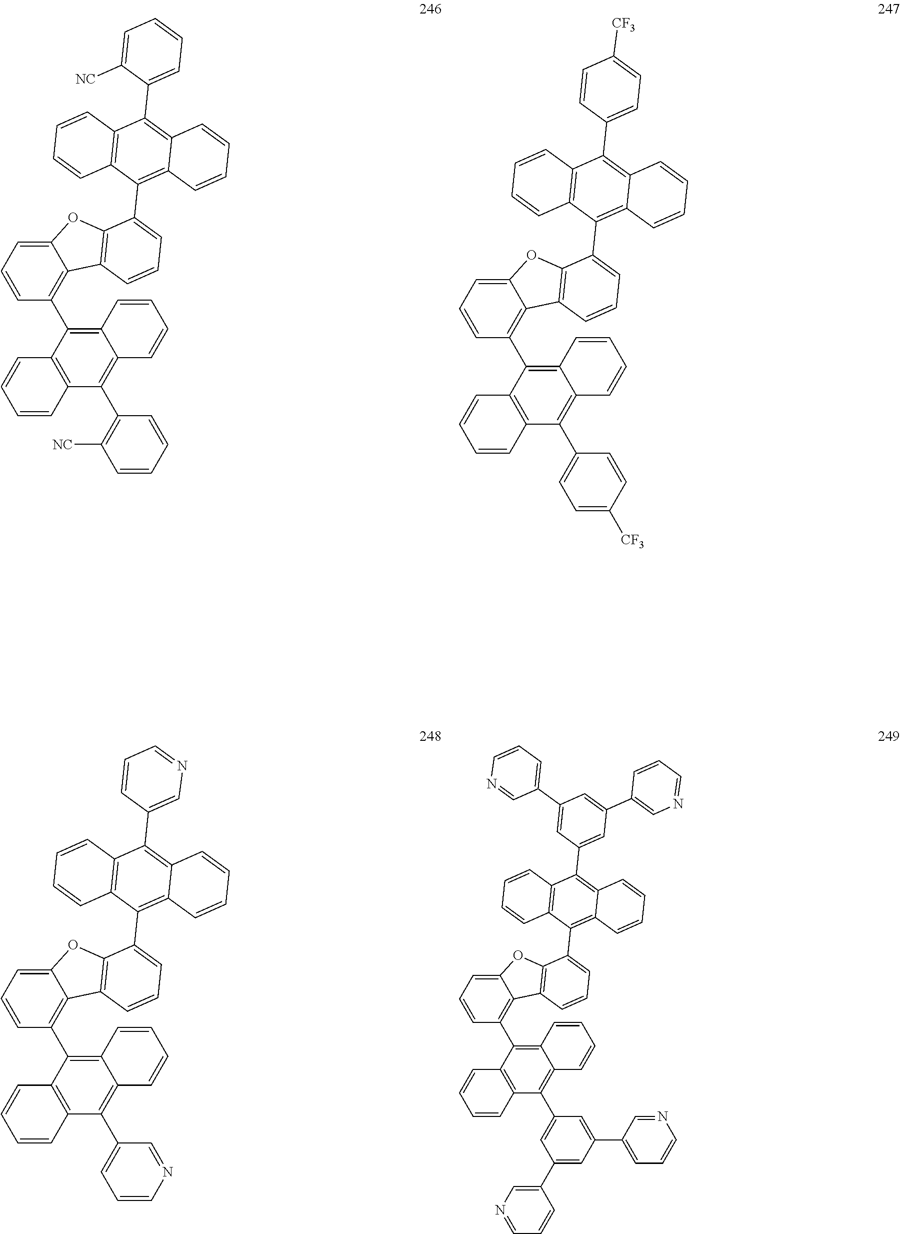 [SMILES: N#Cc1ccccc1-c1c2ccccc2c(-c2cccc3c2oc2cccc(-c4c5ccccc5c(-c5ccccc5C#N)c5ccccc45)c23)c2ccccc12 ; FC(F)(F)c1ccc(-c2c3ccccc3c(-c3cccc4c3oc3cccc(-c5c6ccccc6c(-c6ccc(C(F)(F)F)cc6)c6ccccc56)c34)c3ccccc23)cc1 ; c1cncc(-c2c3ccccc3c(-c3cccc4c3oc3cccc(-c5c6ccccc6c(-c6cccnc6)c6ccccc56)c34)c3ccccc23)c1 ; c1cncc(-c2cc(-c3cccnc3)cc(-c3c4ccccc4c(-c4cccc5c4oc4cccc(-c6c7ccccc7c(-c7cc(-c8cccnc8)cc(-c8cccnc8)c7)c7ccccc67)c45)c4ccccc34)c2)c1]

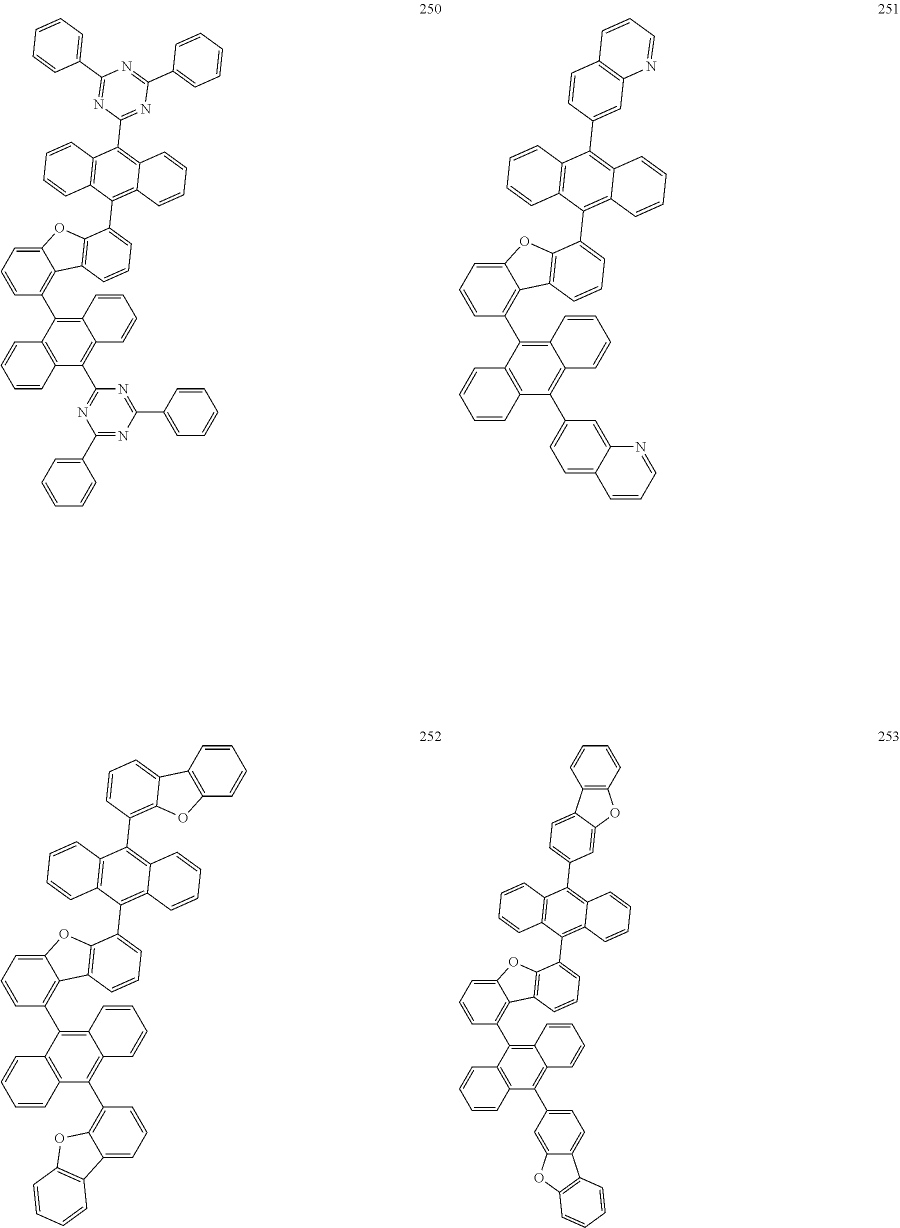 [SMILES: c1ccc(-c2nc(-c3ccccc3)nc(-c3c4ccccc4c(-c4cccc5c4oc4cccc(-c6c7ccccc7c(-c7nc(-c8ccccc8)nc(-c8ccccc8)n7)c7ccccc67)c45)c4ccccc34)n2)cc1 ; c1cnc2cc(-c3c4ccccc4c(-c4cccc5c4oc4cccc(-c6c7ccccc7c(-c7ccc8cccnc8c7)c7ccccc67)c45)c4ccccc34)ccc2c1 ; c1ccc2c(c1)oc1c(-c3c4ccccc4c(-c4cccc5c4oc4cccc(-c6c7ccccc7c(-c7cccc8c7oc7ccccc78)c7ccccc67)c45)c4ccccc34)cccc12 ; c1ccc2c(c1)oc1cc(-c3c4ccccc4c(-c4cccc5c4oc4cccc(-c6c7ccccc7c(-c7ccc8c(c7)oc7ccccc78)c7ccccc67)c45)c4ccccc34)ccc12]

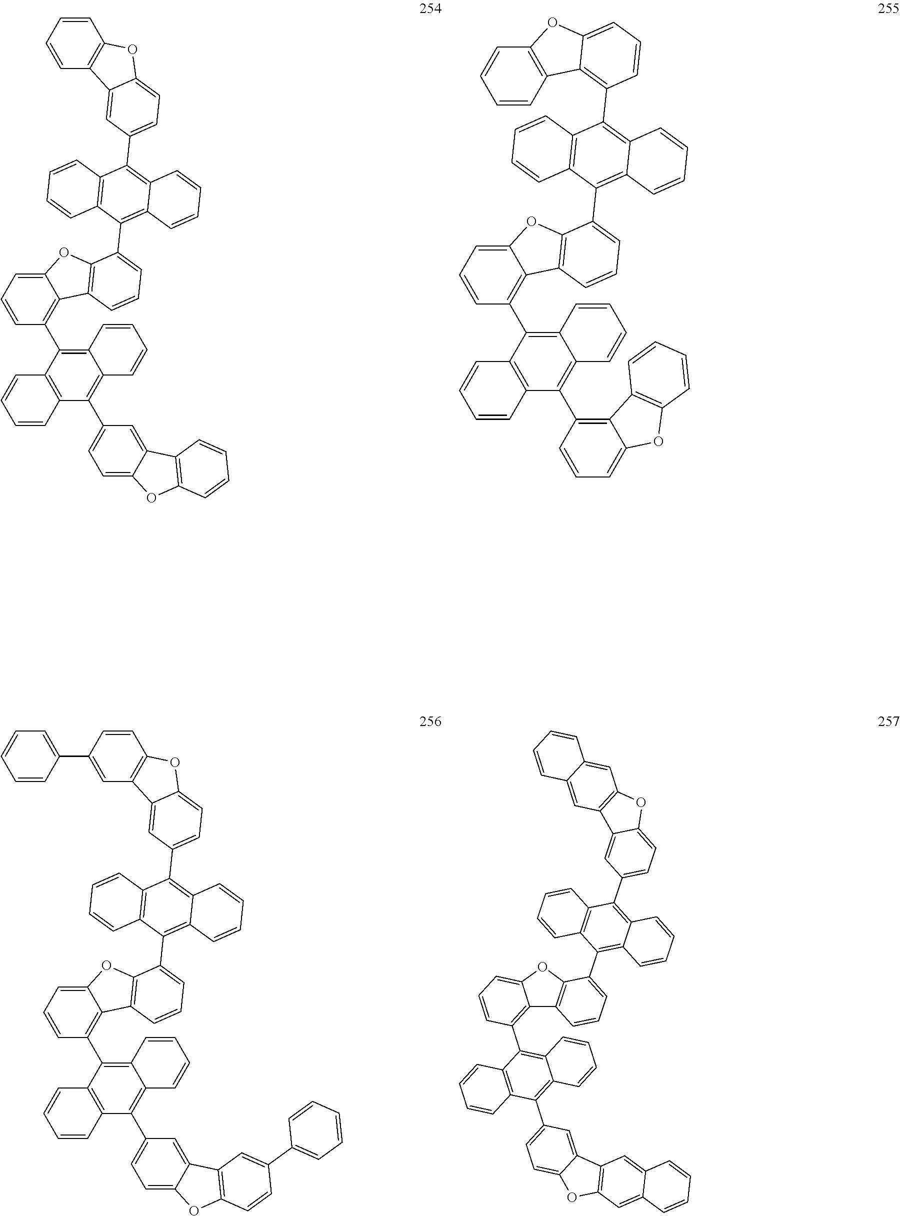

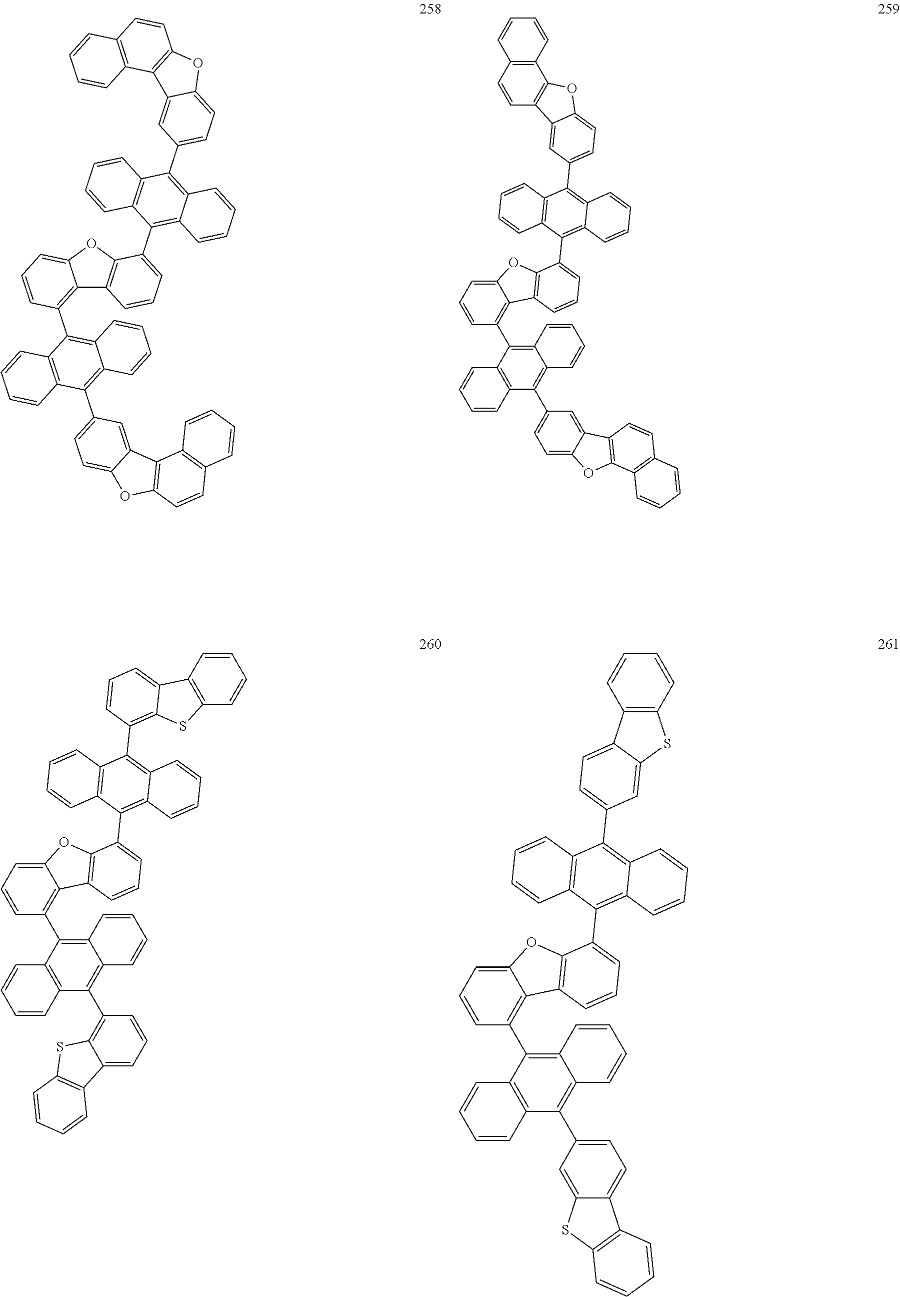 [SMILES: c1ccc2c(c1)ccc1oc3ccc(-c4c5ccccc5c(-c5cccc6c5oc5cccc(-c7c8ccccc8c(-c8ccc9oc%10ccc%11ccccc%11c%10c9c8)c8ccccc78)c56)c5ccccc45)cc3c12 ; c1ccc2c(c1)ccc1c3cc(-c4c5ccccc5c(-c5cccc6c5oc5cccc(-c7c8ccccc8c(-c8ccc9oc%10c%11ccccc%11ccc%10c9c8)c8ccccc78)c56)c5ccccc45)ccc3oc21 ; c1cc(-c2c3ccccc3c(-c3cccc4c3sc3ccccc34)c3ccccc23)c2c(c1)oc1c(-c3c4ccccc4c(-c4cccc5c4sc4ccccc45)c4ccccc34)cccc12 ; c1cc(-c2c3ccccc3c(-c3ccc4c(c3)sc3ccccc34)c3ccccc23)c2c(c1)oc1c(-c3c4ccccc4c(-c4ccc5c(c4)sc4ccccc45)c4ccccc34)cccc12]

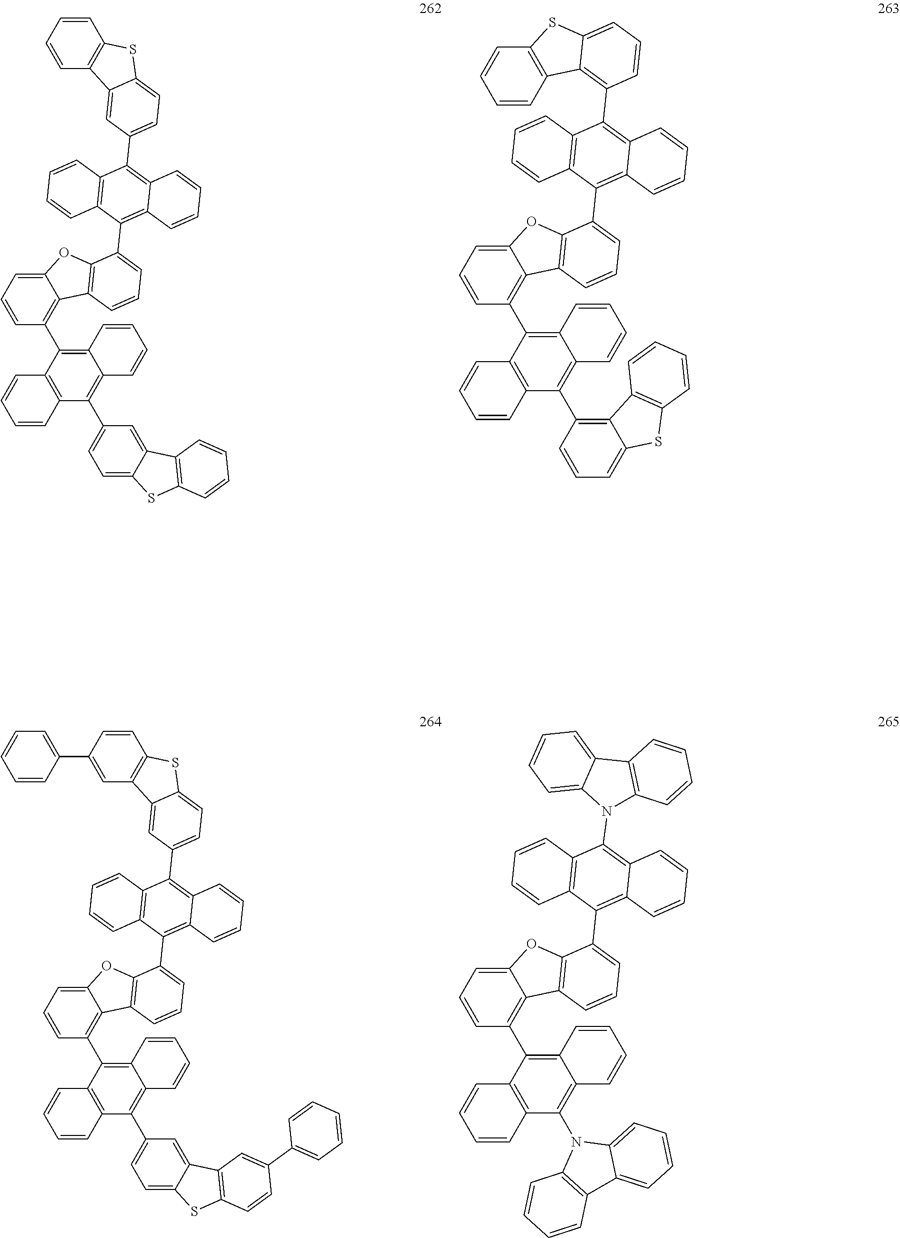 [SMILES: c1cc(-c2c3ccccc3c(-c3ccc4sc5ccccc5c4c3)c3ccccc23)c2c(c1)oc1c(-c3c4ccccc4c(-c4ccc5sc6ccccc6c5c4)c4ccccc34)cccc12 ; c1cc(-c2c3ccccc3c(-c3cccc4sc5ccccc5c34)c3ccccc23)c2c(c1)oc1c(-c3c4ccccc4c(-c4cccc5sc6ccccc6c45)c4ccccc34)cccc12 ; c1ccc(-c2ccc3sc4ccc(-c5c6ccccc6c(-c6cccc7c6oc6cccc(-c8c9ccccc9c(-c9ccc%10sc%11ccc(-c%12ccccc%12)cc%11c%10c9)c9ccccc89)c67)c6ccccc56)cc4c3c2)cc1 ; c1cc(-c2c3ccccc3c(-n3c4ccccc4c4ccccc43)c3ccccc23)c2c(c1)oc1c(-c3c4ccccc4c(-n4c5ccccc5c5ccccc54)c4ccccc34)cccc12]

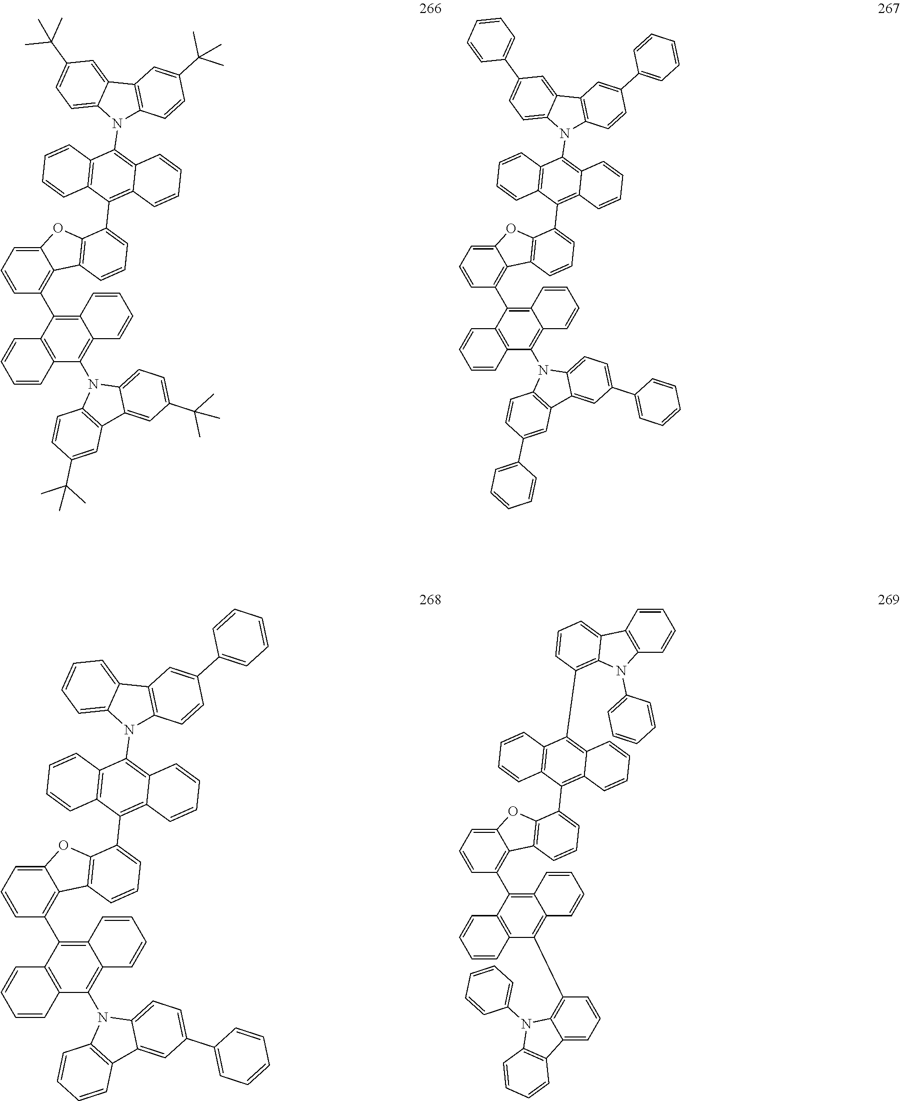

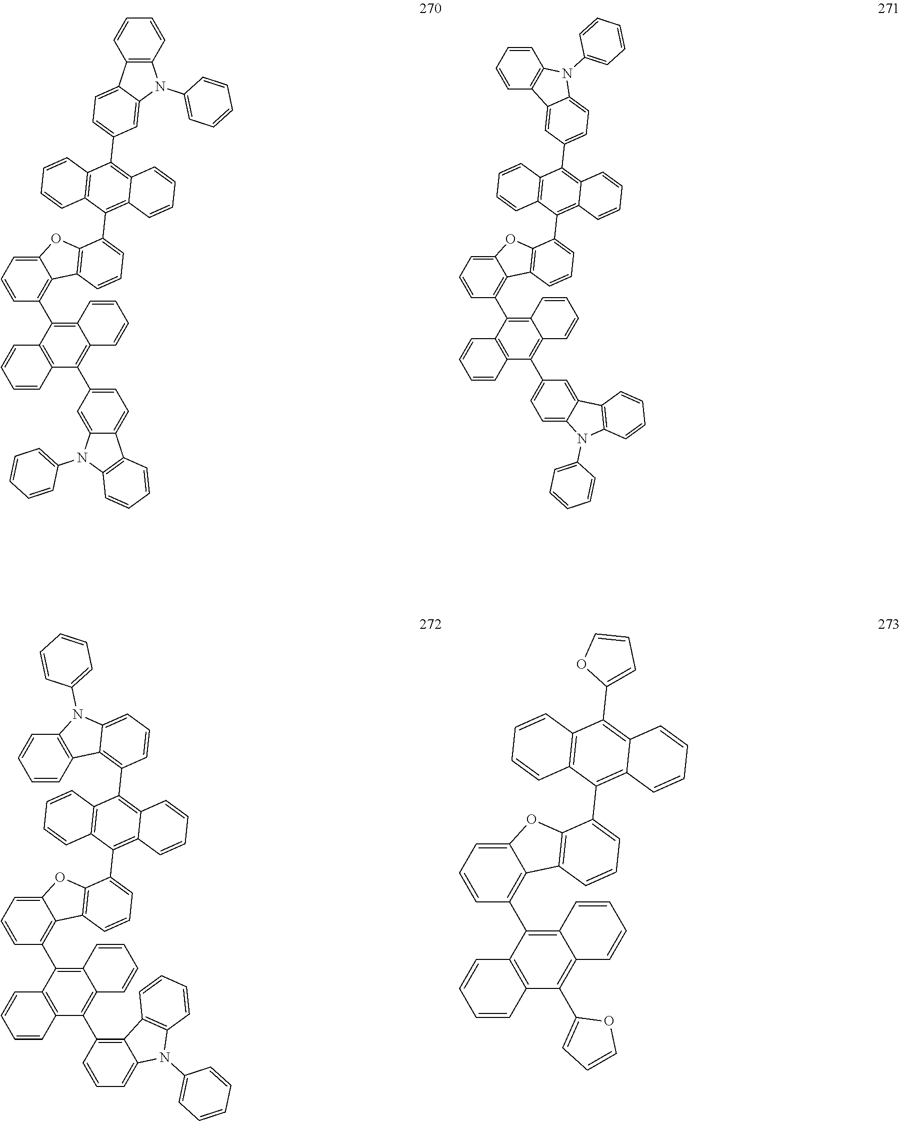 [SMILES: c1ccc(-n2c3ccccc3c3ccc(-c4c5ccccc5c(-c5cccc6c5oc5cccc(-c7c8ccccc8c(-c8ccc9c%10ccccc%10n(-c%10ccccc%10)c9c8)c8ccccc78)c56)c5ccccc45)cc32)cc1 ; c1ccc(-n2c3ccccc3c3cc(-c4c5ccccc5c(-c5cccc6c5oc5cccc(-c7c8ccccc8c(-c8ccc9c(c8)c8ccccc8n9-c8ccccc8)c8ccccc78)c56)c5ccccc45)ccc32)cc1 ; c1ccc(-n2c3ccccc3c3c(-c4c5ccccc5c(-c5cccc6c5oc5cccc(-c7c8ccccc8c(-c8cccc9c8c8ccccc8n9-c8ccccc8)c8ccccc78)c56)c5ccccc45)cccc32)cc1 ; c1coc(-c2c3ccccc3c(-c3cccc4c3oc3cccc(-c5c6ccccc6c(-c6ccco6)c6ccccc56)c34)c3ccccc23)c1]

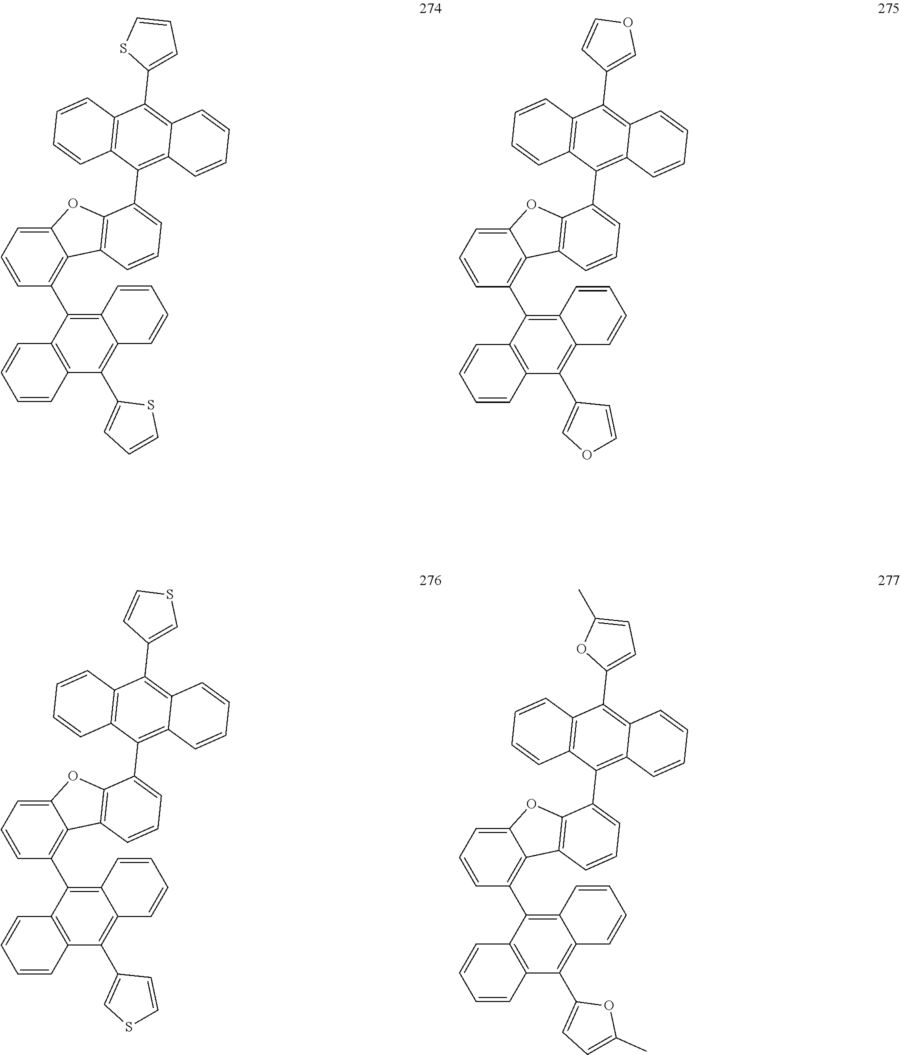 [SMILES: c1csc(-c2c3ccccc3c(-c3cccc4c3oc3cccc(-c5c6ccccc6c(-c6cccs6)c6ccccc56)c34)c3ccccc23)c1 ; c1cc(-c2c3ccccc3c(-c3ccoc3)c3ccccc23)c2c(c1)oc1c(-c3c4ccccc4c(-c4ccoc4)c4ccccc34)cccc12 ; c1cc(-c2c3ccccc3c(-c3ccsc3)c3ccccc23)c2c(c1)oc1c(-c3c4ccccc4c(-c4ccsc4)c4ccccc34)cccc12 ; Cc1ccc(-c2c3ccccc3c(-c3cccc4c3oc3cccc(-c5c6ccccc6c(-c6ccc(C)o6)c6ccccc56)c34)c3ccccc23)o1]

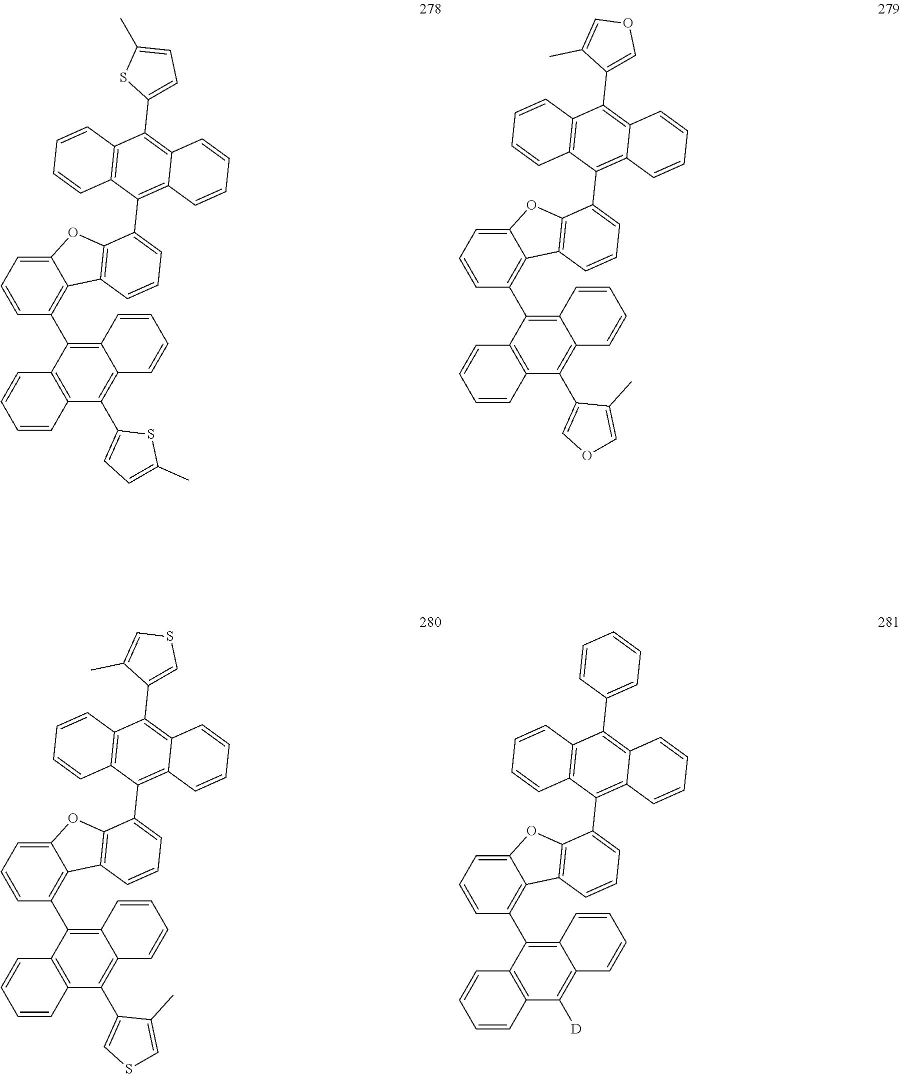 [SMILES: Cc1ccc(-c2c3ccccc3c(-c3cccc4c3oc3cccc(-c5c6ccccc6c(-c6ccc(C)s6)c6ccccc56)c34)c3ccccc23)s1 ; Cc1cocc1-c1c2ccccc2c(-c2cccc3c2oc2cccc(-c4c5ccccc5c(-c5cocc5C)c5ccccc45)c23)c2ccccc12 ; Cc1cscc1-c1c2ccccc2c(-c2cccc3c2oc2cccc(-c4c5ccccc5c(-c5cscc5C)c5ccccc45)c23)c2ccccc12 ; [2H]c1c2ccccc2c(-c2cccc3oc4c(-c5c6ccccc6c(-c6ccccc6)c6ccccc56)cccc4c23)c2ccccc12]

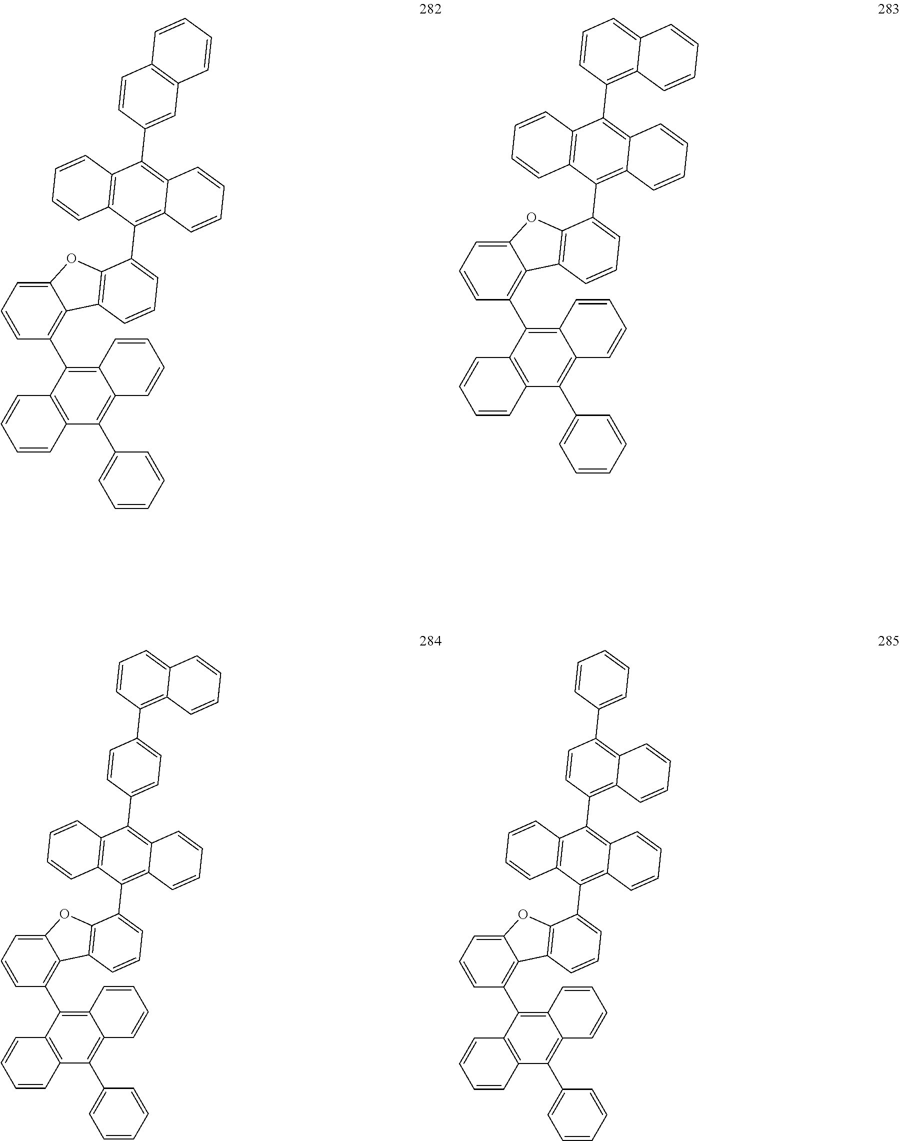 [SMILES: c1ccc(-c2c3ccccc3c(-c3cccc4oc5c(-c6c7ccccc7c(-c7ccc8ccccc8c7)c7ccccc67)cccc5c34)c3ccccc23)cc1 ; c1ccc(-c2c3ccccc3c(-c3cccc4oc5c(-c6c7ccccc7c(-c7cccc8ccccc78)c7ccccc67)cccc5c34)c3ccccc23)cc1 ; c1ccc(-c2c3ccccc3c(-c3cccc4oc5c(-c6c7ccccc7c(-c7ccc(-c8cccc9ccccc89)cc7)c7ccccc67)cccc5c34)c3ccccc23)cc1 ; c1ccc(-c2ccc(-c3c4ccccc4c(-c4cccc5c4oc4cccc(-c6c7ccccc7c(-c7ccccc7)c7ccccc67)c45)c4ccccc34)c3ccccc23)cc1]

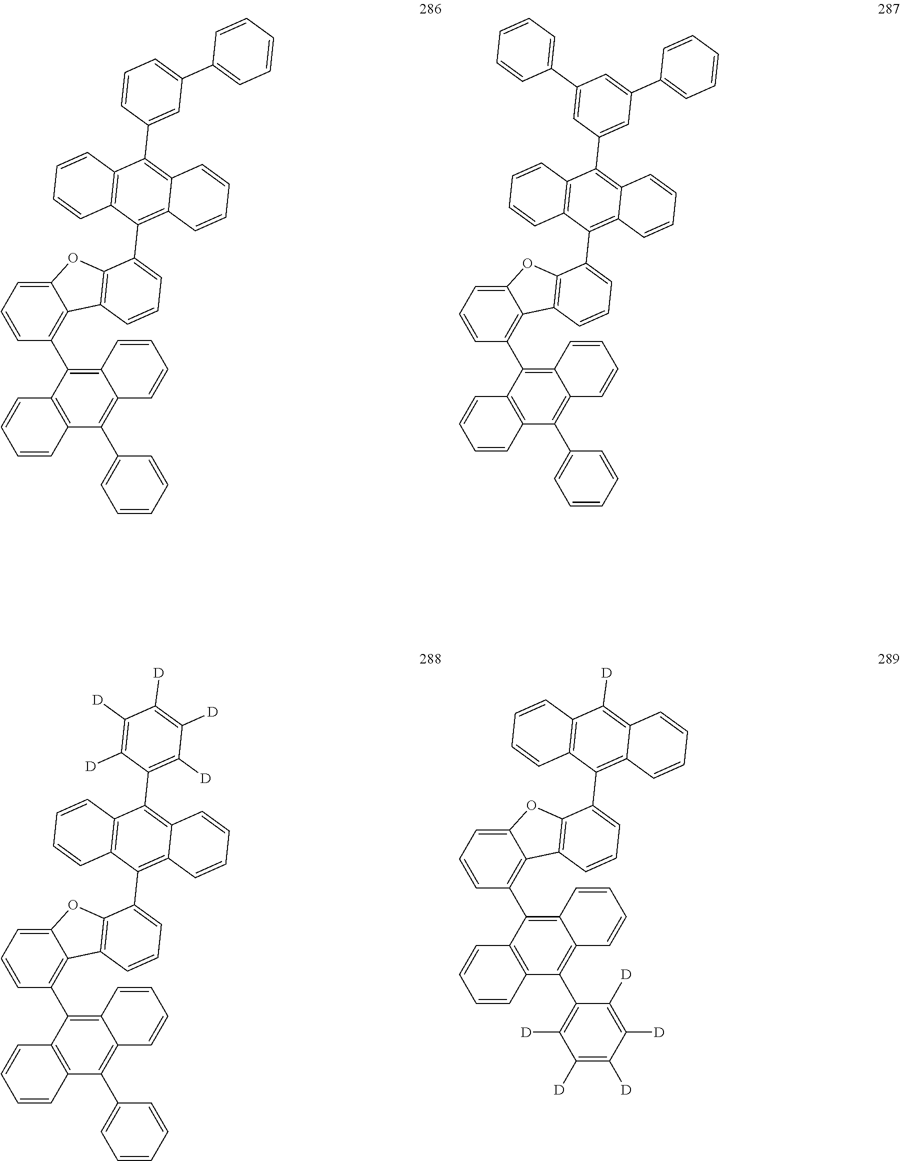 [SMILES: c1ccc(-c2cccc(-c3c4ccccc4c(-c4cccc5c4oc4cccc(-c6c7ccccc7c(-c7ccccc7)c7ccccc67)c45)c4ccccc34)c2)cc1 ; c1ccc(-c2cc(-c3ccccc3)cc(-c3c4ccccc4c(-c4cccc5c4oc4cccc(-c6c7ccccc7c(-c7ccccc7)c7ccccc67)c45)c4ccccc34)c2)cc1 ; [2H]c1c([2H])c([2H])c(-c2c3ccccc3c(-c3cccc4c3oc3cccc(-c5c6ccccc6c(-c6ccccc6)c6ccccc56)c34)c3ccccc23)c([2H])c1[2H] ; [2H]c1c([2H])c([2H])c(-c2c3ccccc3c(-c3cccc4oc5c(-c6c7ccccc7c([2H])c7ccccc67)cccc5c34)c3ccccc23)c([2H])c1[2H]]

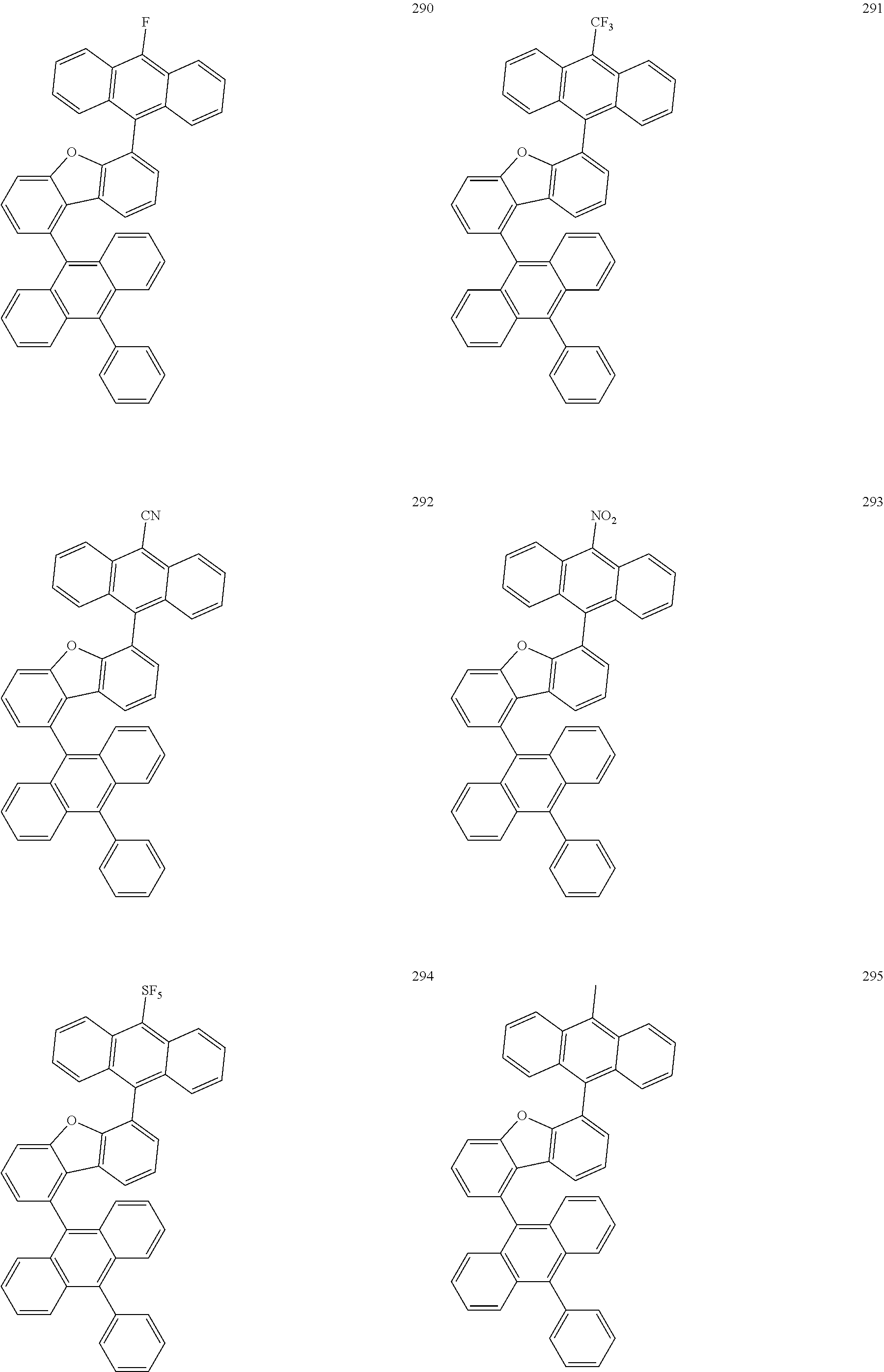

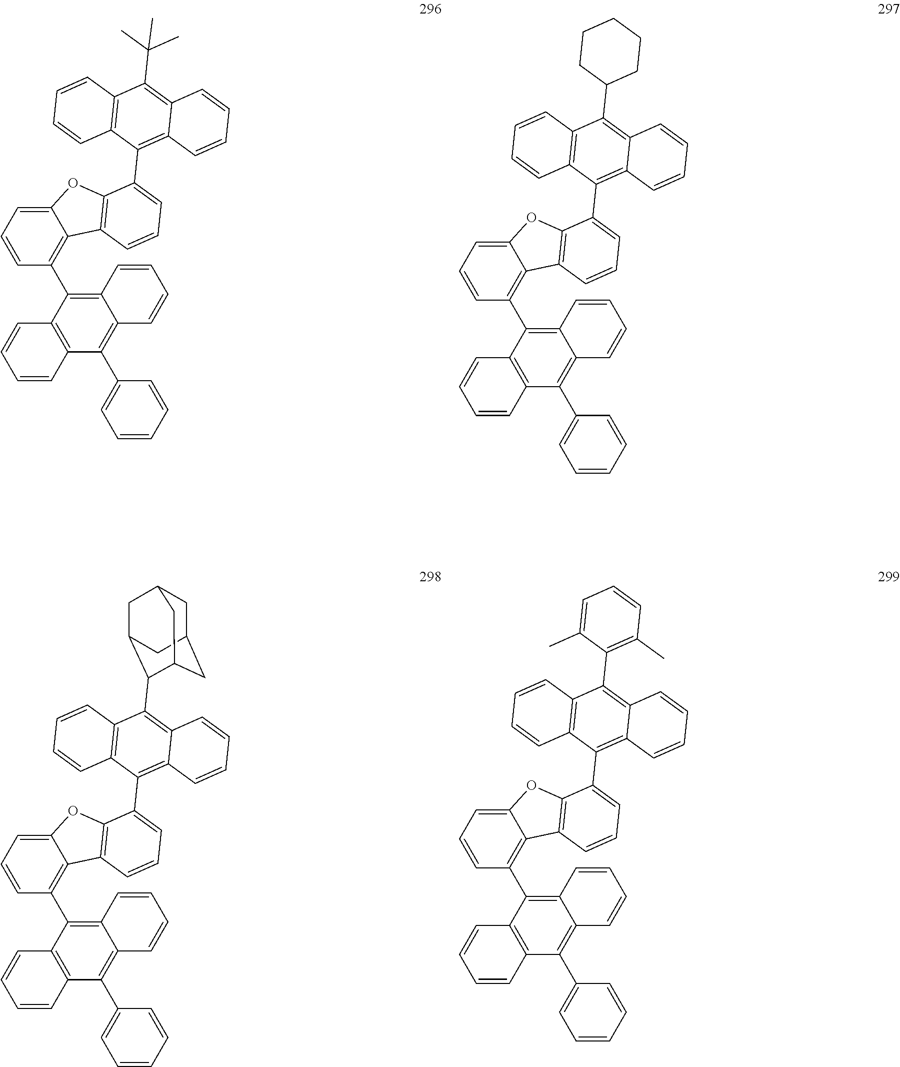 [SMILES: CC(C)(C)c1c2ccccc2c(-c2cccc3c2oc2cccc(-c4c5ccccc5c(-c5ccccc5)c5ccccc45)c23)c2ccccc12 ; c1ccc(-c2c3ccccc3c(-c3cccc4oc5c(-c6c7ccccc7c(C7CCCCC7)c7ccccc67)cccc5c34)c3ccccc23)cc1 ; c1ccc(-c2c3ccccc3c(-c3cccc4oc5c(-c6c7ccccc7c(C7C8CC9CC(C8)CC7C9)c7ccccc67)cccc5c34)c3ccccc23)cc1 ; Cc1cccc(C)c1-c1c2ccccc2c(-c2cccc3c2oc2cccc(-c4c5ccccc5c(-c5ccccc5)c5ccccc45)c23)c2ccccc12]

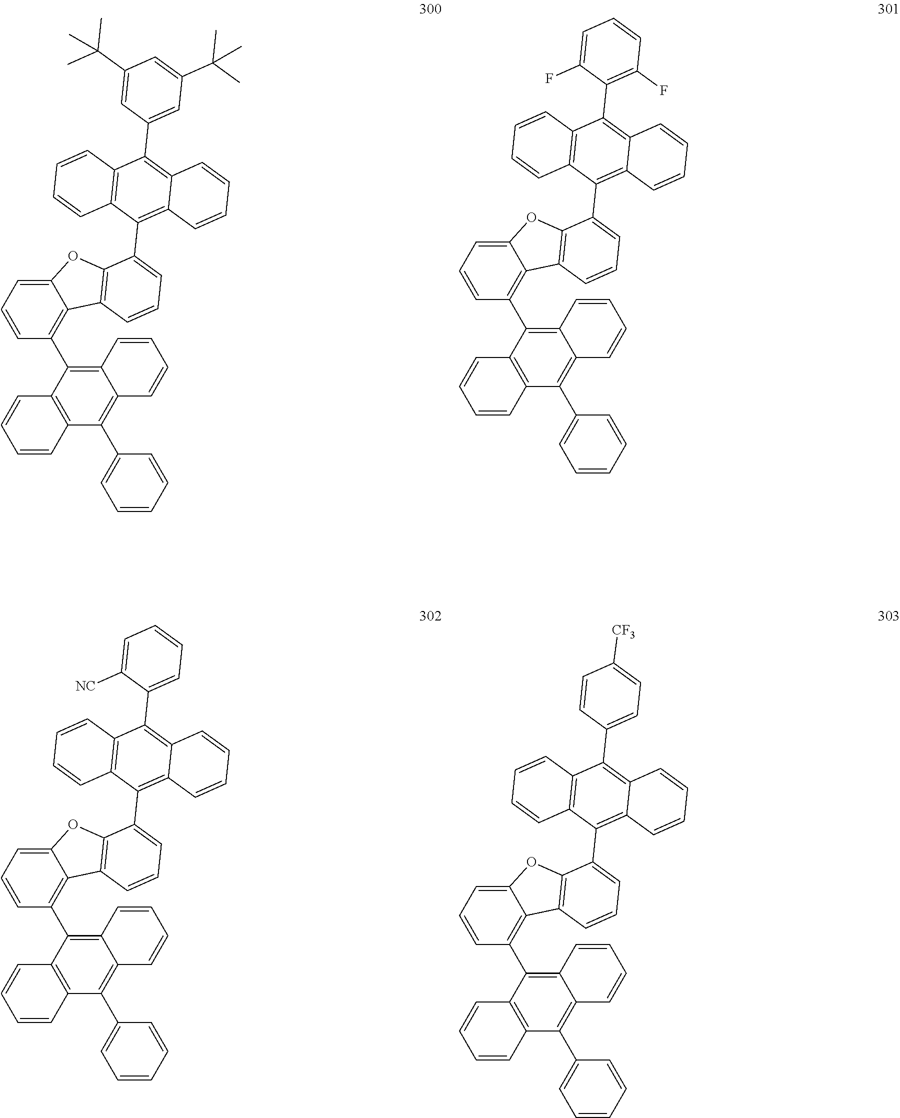 [SMILES: CC(C)(C)c1cc(-c2c3ccccc3c(-c3cccc4c3oc3cccc(-c5c6ccccc6c(-c6ccccc6)c6ccccc56)c34)c3ccccc23)cc(C(C)(C)C)c1 ; Fc1cccc(F)c1-c1c2ccccc2c(-c2cccc3c2oc2cccc(-c4c5ccccc5c(-c5ccccc5)c5ccccc45)c23)c2ccccc12 ; N#Cc1ccccc1-c1c2ccccc2c(-c2cccc3c2oc2cccc(-c4c5ccccc5c(-c5ccccc5)c5ccccc45)c23)c2ccccc12 ; FC(F)(F)c1ccc(-c2c3ccccc3c(-c3cccc4c3oc3cccc(-c5c6ccccc6c(-c6ccccc6)c6ccccc56)c34)c3ccccc23)cc1]

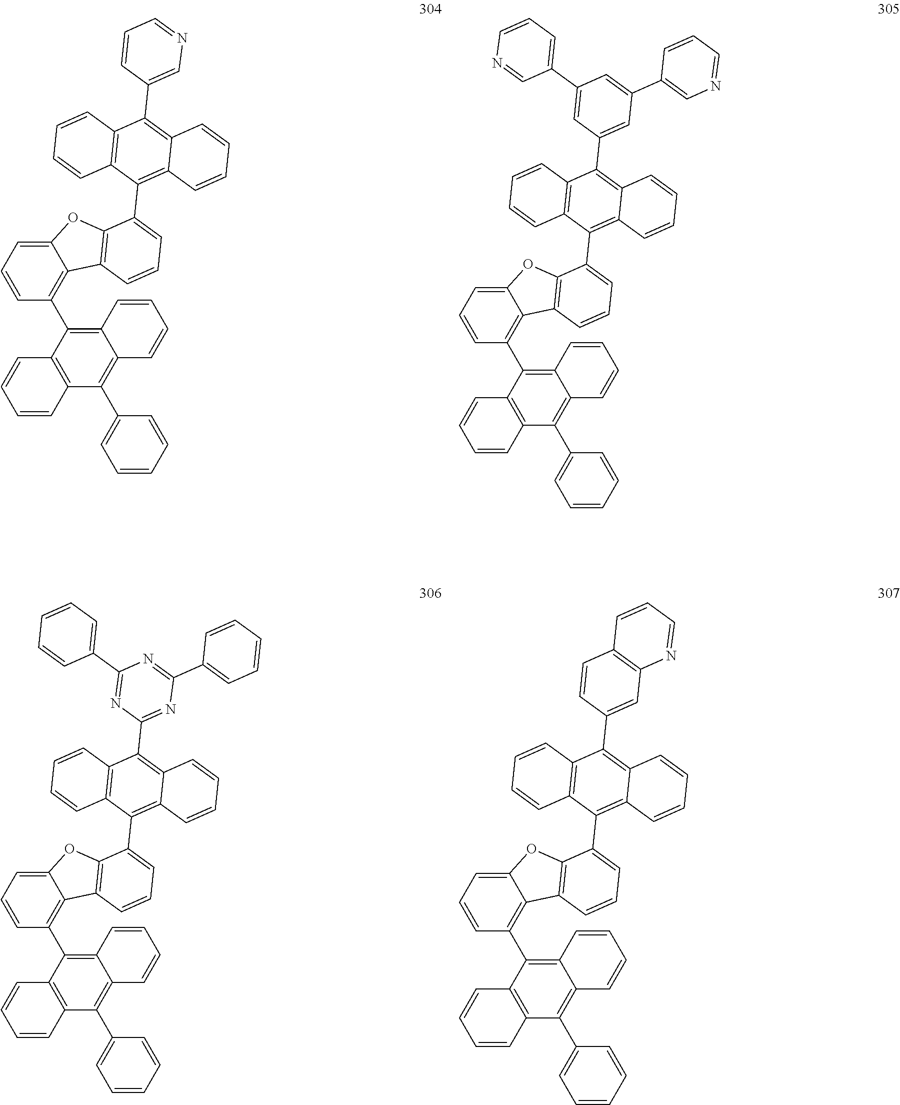 [SMILES: c1ccc(-c2c3ccccc3c(-c3cccc4oc5c(-c6c7ccccc7c(-c7cccnc7)c7ccccc67)cccc5c34)c3ccccc23)cc1 ; c1ccc(-c2c3ccccc3c(-c3cccc4oc5c(-c6c7ccccc7c(-c7cc(-c8cccnc8)cc(-c8cccnc8)c7)c7ccccc67)cccc5c34)c3ccccc23)cc1 ; c1ccc(-c2nc(-c3ccccc3)nc(-c3c4ccccc4c(-c4cccc5c4oc4cccc(-c6c7ccccc7c(-c7ccccc7)c7ccccc67)c45)c4ccccc34)n2)cc1 ; c1ccc(-c2c3ccccc3c(-c3cccc4oc5c(-c6c7ccccc7c(-c7ccc8cccnc8c7)c7ccccc67)cccc5c34)c3ccccc23)cc1]

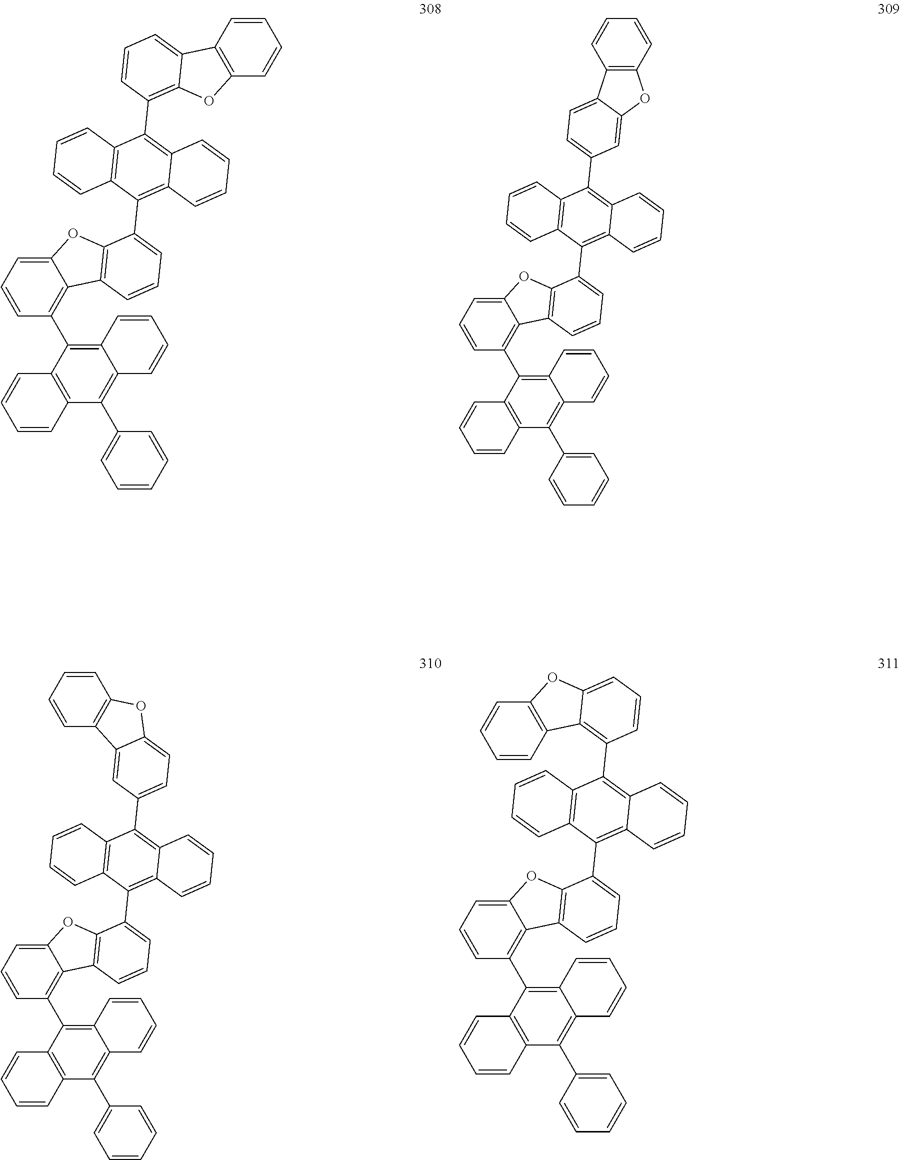 [SMILES: c1ccc(-c2c3ccccc3c(-c3cccc4oc5c(-c6c7ccccc7c(-c7cccc8c7oc7ccccc78)c7ccccc67)cccc5c34)c3ccccc23)cc1 ; c1ccc(-c2c3ccccc3c(-c3cccc4oc5c(-c6c7ccccc7c(-c7ccc8c(c7)oc7ccccc78)c7ccccc67)cccc5c34)c3ccccc23)cc1 ; c1ccc(-c2c3ccccc3c(-c3cccc4oc5c(-c6c7ccccc7c(-c7ccc8oc9ccccc9c8c7)c7ccccc67)cccc5c34)c3ccccc23)cc1 ; c1ccc(-c2c3ccccc3c(-c3cccc4oc5c(-c6c7ccccc7c(-c7cccc8oc9ccccc9c78)c7ccccc67)cccc5c34)c3ccccc23)cc1]

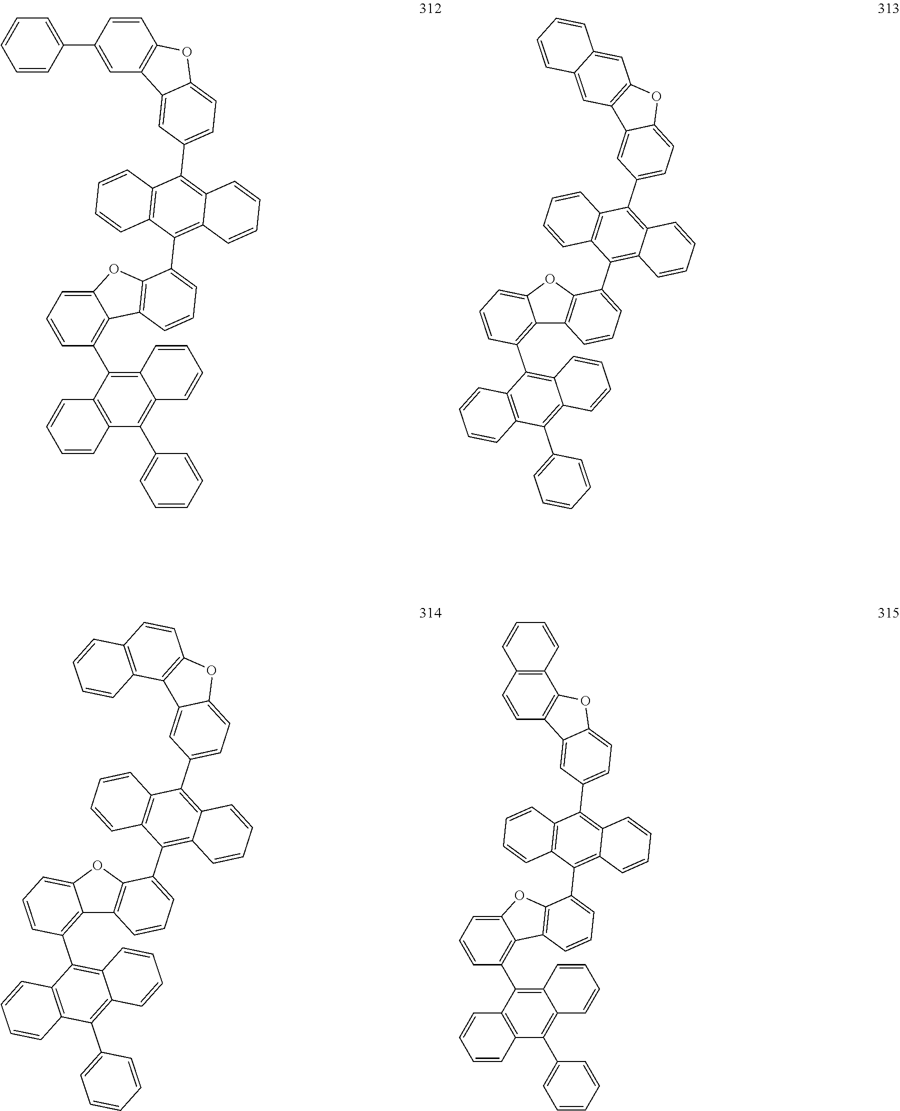 [SMILES: c1ccc(-c2ccc3oc4ccc(-c5c6ccccc6c(-c6cccc7c6oc6cccc(-c8c9ccccc9c(-c9ccccc9)c9ccccc89)c67)c6ccccc56)cc4c3c2)cc1 ; c1ccc(-c2c3ccccc3c(-c3cccc4oc5c(-c6c7ccccc7c(-c7ccc8oc9cc%10ccccc%10cc9c8c7)c7ccccc67)cccc5c34)c3ccccc23)cc1 ; c1ccc(-c2c3ccccc3c(-c3cccc4oc5c(-c6c7ccccc7c(-c7ccc8oc9ccc%10ccccc%10c9c8c7)c7ccccc67)cccc5c34)c3ccccc23)cc1 ; c1ccc(-c2c3ccccc3c(-c3cccc4oc5c(-c6c7ccccc7c(-c7ccc8oc9c%10ccccc%10ccc9c8c7)c7ccccc67)cccc5c34)c3ccccc23)cc1]

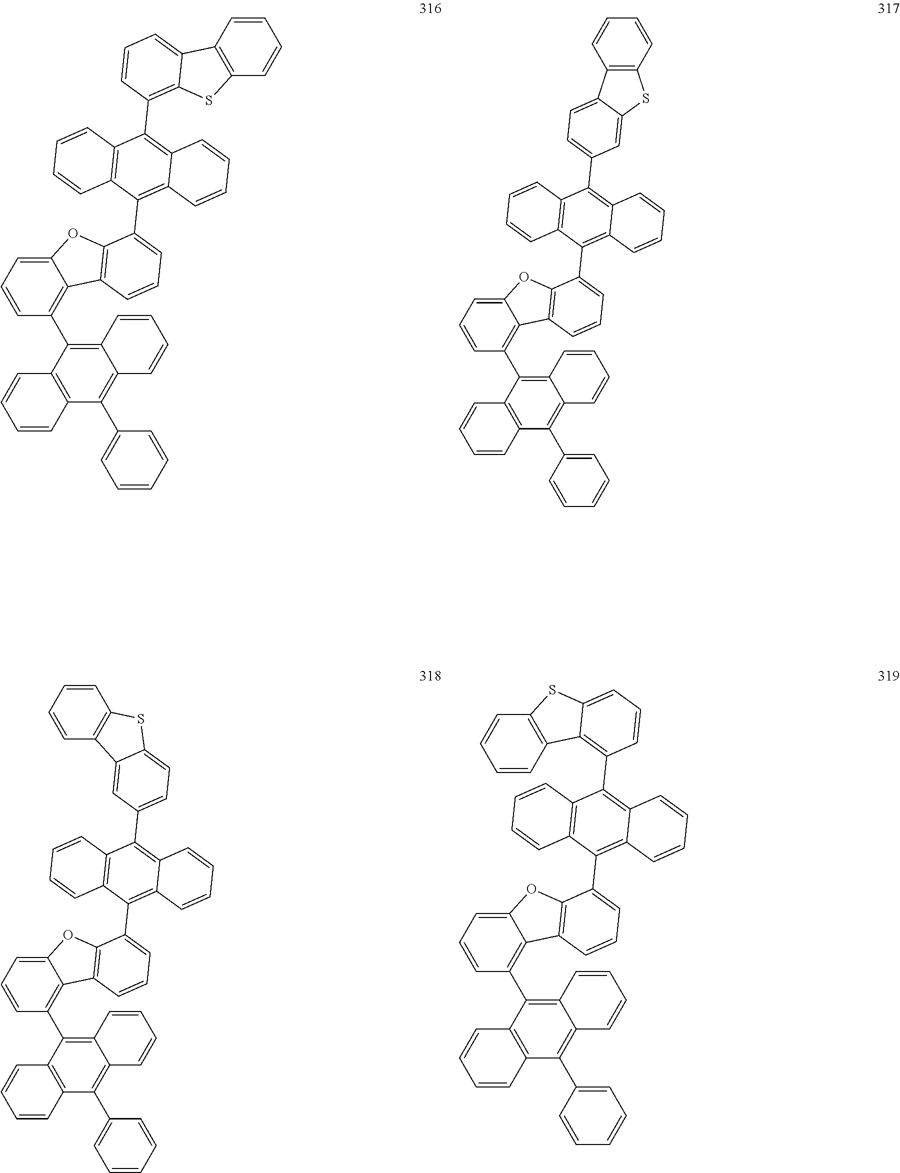 [SMILES: c1ccc(-c2c3ccccc3c(-c3cccc4oc5c(-c6c7ccccc7c(-c7cccc8c7sc7ccccc78)c7ccccc67)cccc5c34)c3ccccc23)cc1 ; c1ccc(-c2c3ccccc3c(-c3cccc4oc5c(-c6c7ccccc7c(-c7ccc8c(c7)sc7ccccc78)c7ccccc67)cccc5c34)c3ccccc23)cc1 ; c1ccc(-c2c3ccccc3c(-c3cccc4oc5c(-c6c7ccccc7c(-c7ccc8sc9ccccc9c8c7)c7ccccc67)cccc5c34)c3ccccc23)cc1 ; c1ccc(-c2c3ccccc3c(-c3cccc4oc5c(-c6c7ccccc7c(-c7cccc8sc9ccccc9c78)c7ccccc67)cccc5c34)c3ccccc23)cc1]

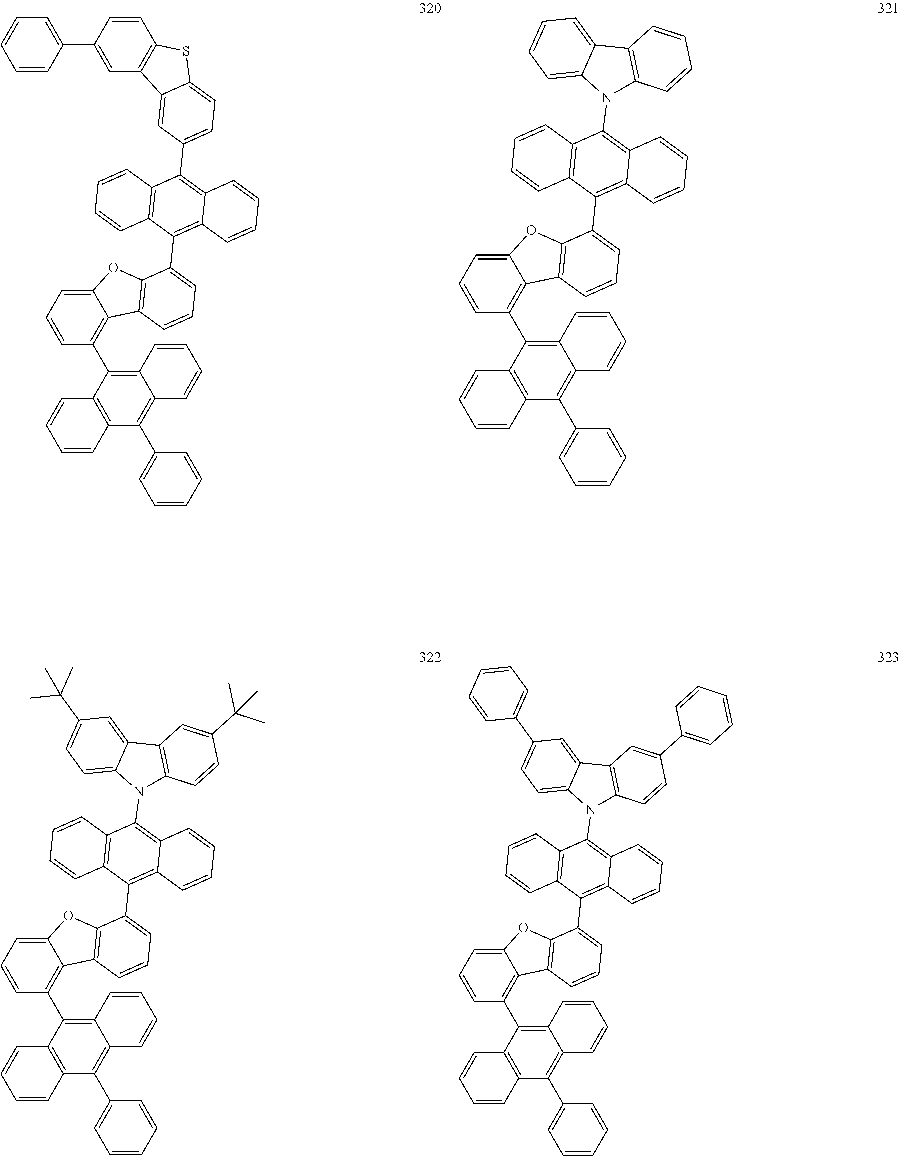 [SMILES: c1ccc(-c2ccc3sc4ccc(-c5c6ccccc6c(-c6cccc7c6oc6cccc(-c8c9ccccc9c(-c9ccccc9)c9ccccc89)c67)c6ccccc56)cc4c3c2)cc1 ; c1ccc(-c2c3ccccc3c(-c3cccc4oc5c(-c6c7ccccc7c(-n7c8ccccc8c8ccccc87)c7ccccc67)cccc5c34)c3ccccc23)cc1 ; CC(C)(C)c1ccc2c(c1)c1cc(C(C)(C)C)ccc1n2-c1c2ccccc2c(-c2cccc3c2oc2cccc(-c4c5ccccc5c(-c5ccccc5)c5ccccc45)c23)c2ccccc12 ; c1ccc(-c2ccc3c(c2)c2cc(-c4ccccc4)ccc2n3-c2c3ccccc3c(-c3cccc4c3oc3cccc(-c5c6ccccc6c(-c6ccccc6)c6ccccc56)c34)c3ccccc23)cc1]

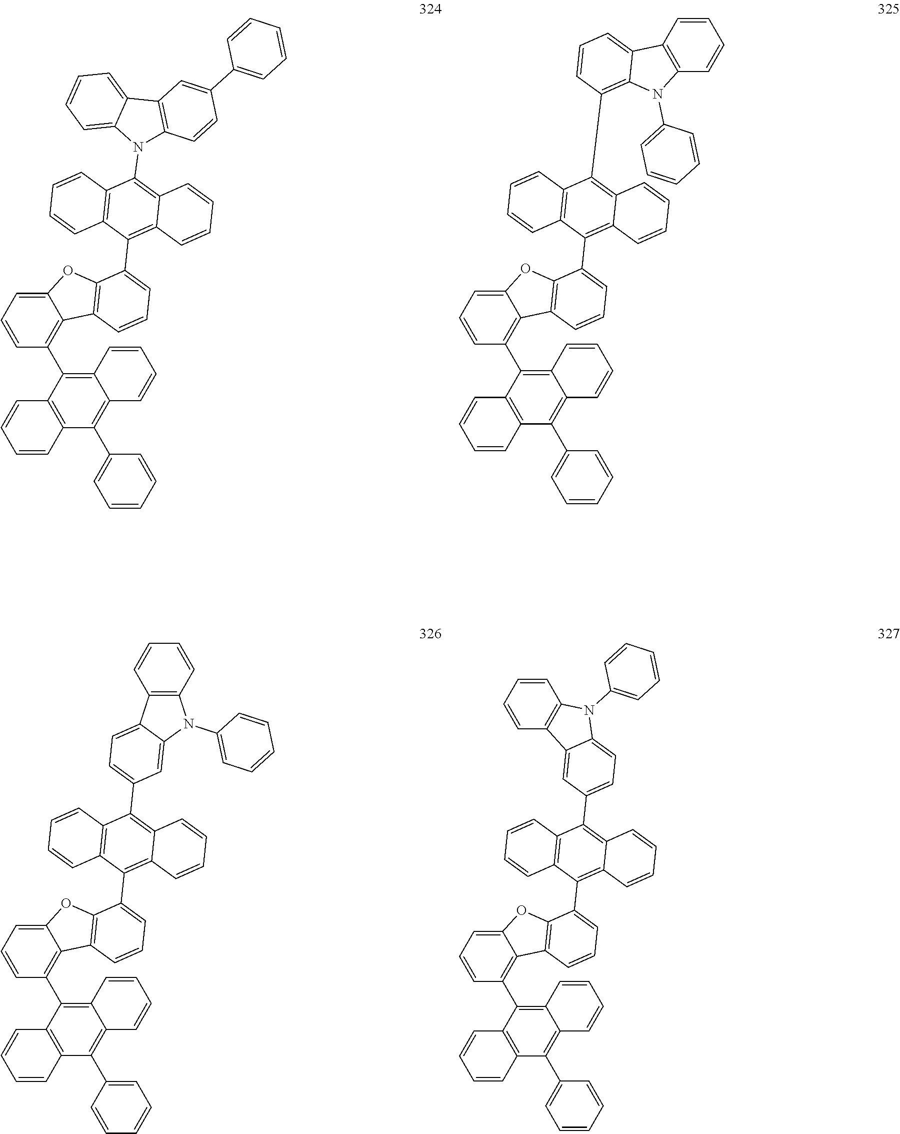 [SMILES: c1ccc(-c2ccc3c(c2)c2ccccc2n3-c2c3ccccc3c(-c3cccc4c3oc3cccc(-c5c6ccccc6c(-c6ccccc6)c6ccccc56)c34)c3ccccc23)cc1 ; c1ccc(-c2c3ccccc3c(-c3cccc4oc5c(-c6c7ccccc7c(-c7cccc8c9ccccc9n(-c9ccccc9)c78)c7ccccc67)cccc5c34)c3ccccc23)cc1 ; c1ccc(-c2c3ccccc3c(-c3cccc4oc5c(-c6c7ccccc7c(-c7ccc8c9ccccc9n(-c9ccccc9)c8c7)c7ccccc67)cccc5c34)c3ccccc23)cc1 ; c1ccc(-c2c3ccccc3c(-c3cccc4oc5c(-c6c7ccccc7c(-c7ccc8c(c7)c7ccccc7n8-c7ccccc7)c7ccccc67)cccc5c34)c3ccccc23)cc1]

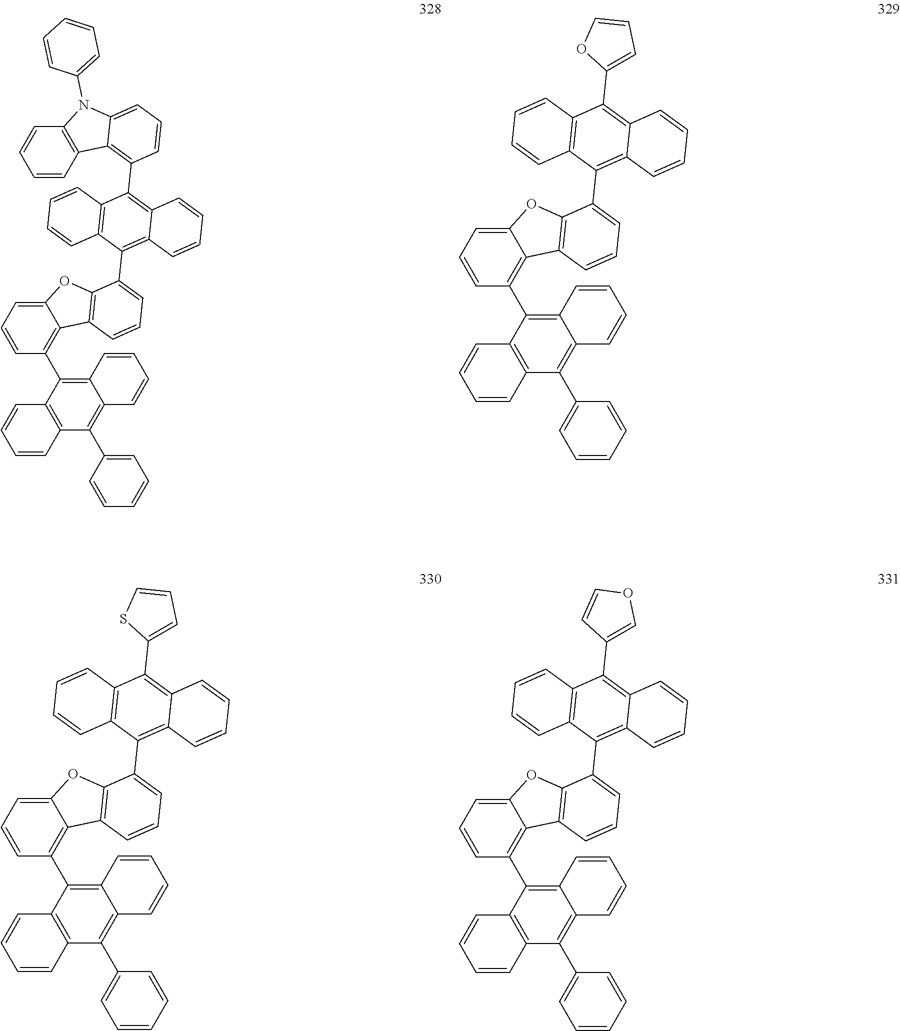 [SMILES: c1ccc(-c2c3ccccc3c(-c3cccc4oc5c(-c6c7ccccc7c(-c7cccc8c7c7ccccc7n8-c7ccccc7)c7ccccc67)cccc5c34)c3ccccc23)cc1 ; c1ccc(-c2c3ccccc3c(-c3cccc4oc5c(-c6c7ccccc7c(-c7ccco7)c7ccccc67)cccc5c34)c3ccccc23)cc1 ; c1ccc(-c2c3ccccc3c(-c3cccc4oc5c(-c6c7ccccc7c(-c7cccs7)c7ccccc67)cccc5c34)c3ccccc23)cc1 ; c1ccc(-c2c3ccccc3c(-c3cccc4oc5c(-c6c7ccccc7c(-c7ccoc7)c7ccccc67)cccc5c34)c3ccccc23)cc1]

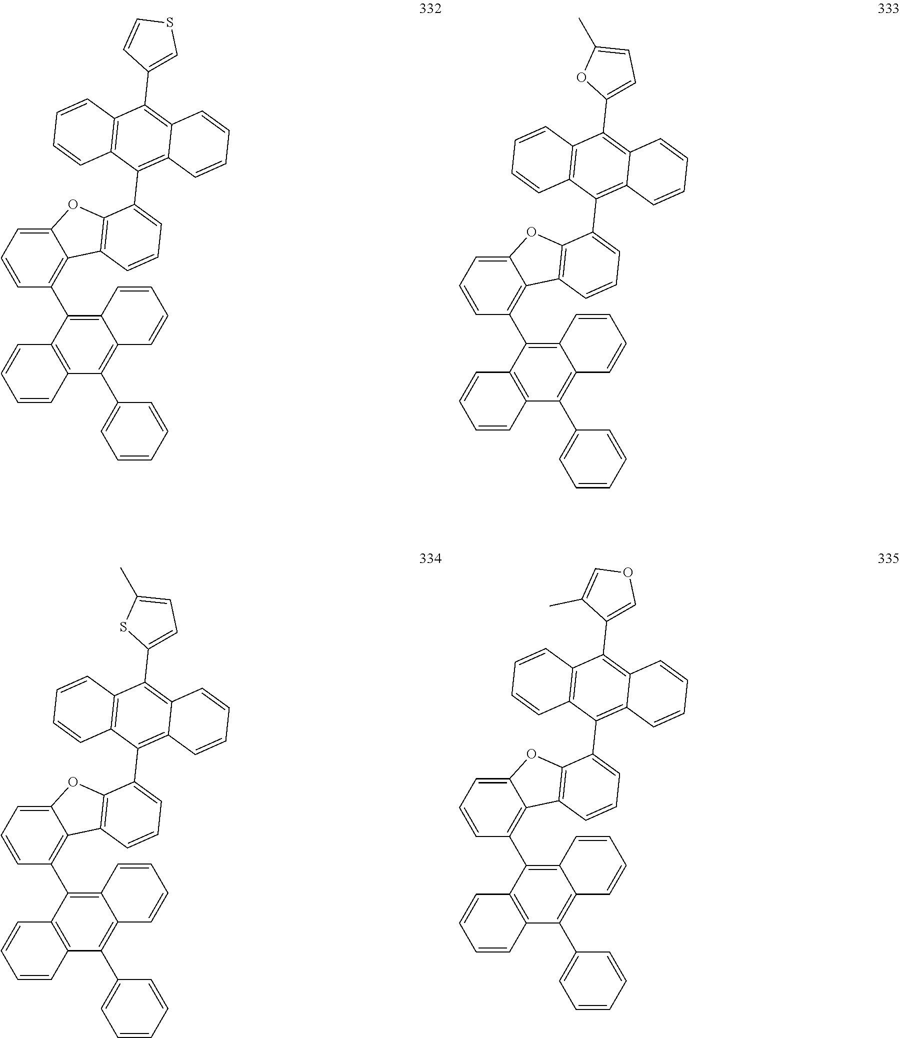 [SMILES: c1ccc(-c2c3ccccc3c(-c3cccc4oc5c(-c6c7ccccc7c(-c7ccsc7)c7ccccc67)cccc5c34)c3ccccc23)cc1 ; Cc1ccc(-c2c3ccccc3c(-c3cccc4c3oc3cccc(-c5c6ccccc6c(-c6ccccc6)c6ccccc56)c34)c3ccccc23)o1 ; Cc1ccc(-c2c3ccccc3c(-c3cccc4c3oc3cccc(-c5c6ccccc6c(-c6ccccc6)c6ccccc56)c34)c3ccccc23)s1 ; Cc1cocc1-c1c2ccccc2c(-c2cccc3c2oc2cccc(-c4c5ccccc5c(-c5ccccc5)c5ccccc45)c23)c2ccccc12]

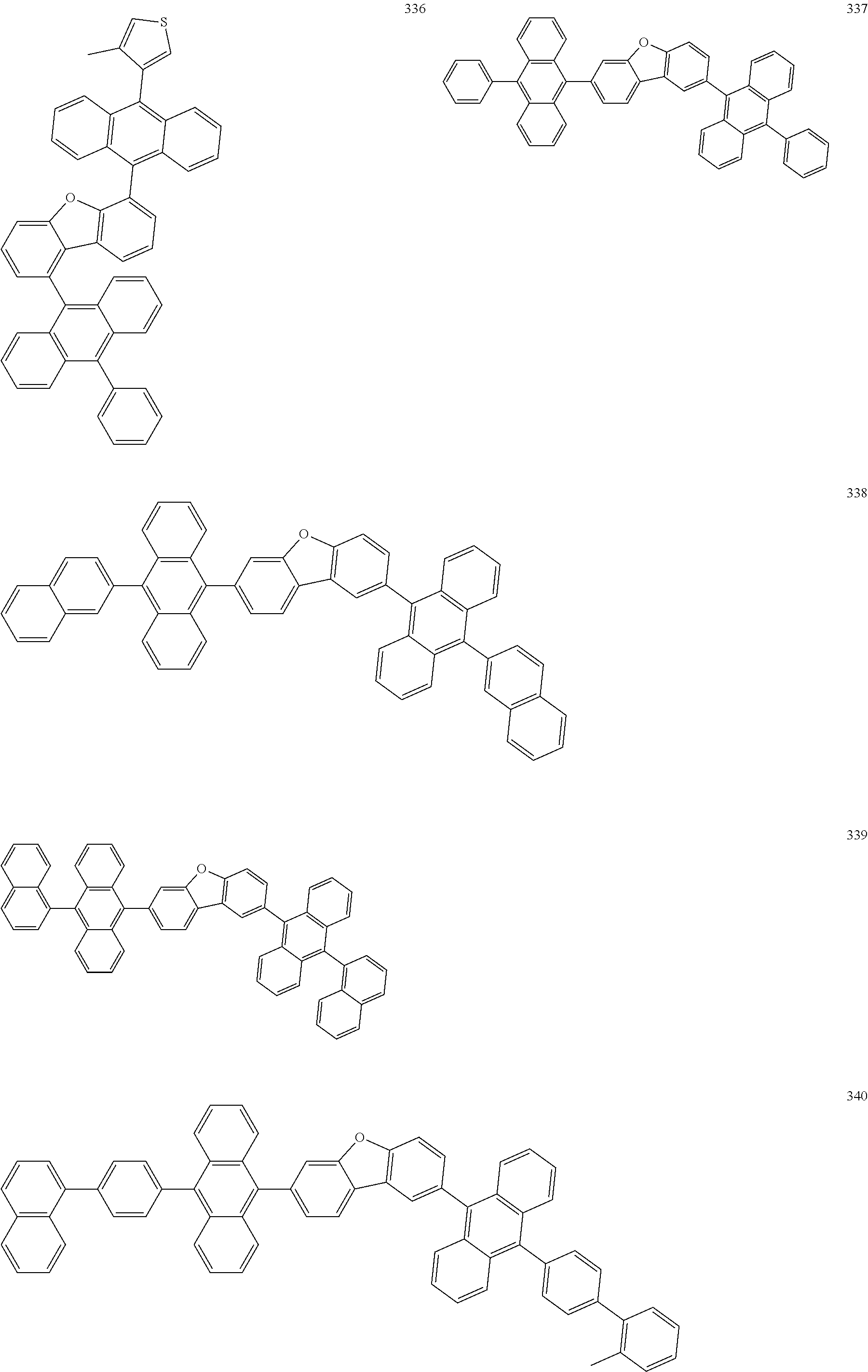

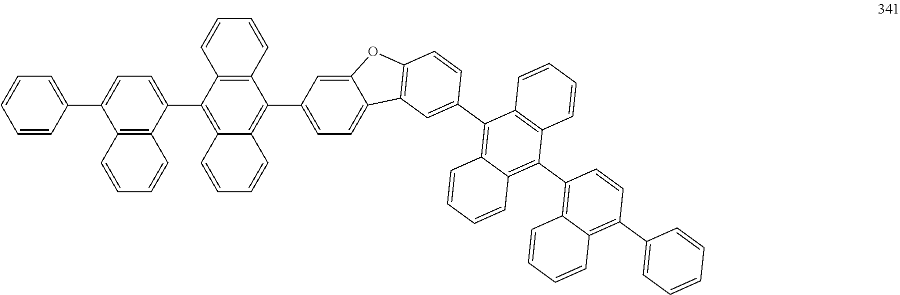 [SMILES: c1ccc(-c2ccc(-c3c4ccccc4c(-c4ccc5c(c4)oc4ccc(-c6c7ccccc7c(-c7ccc(-c8ccccc8)c8ccccc78)c7ccccc67)cc45)c4ccccc34)c3ccccc23)cc1]

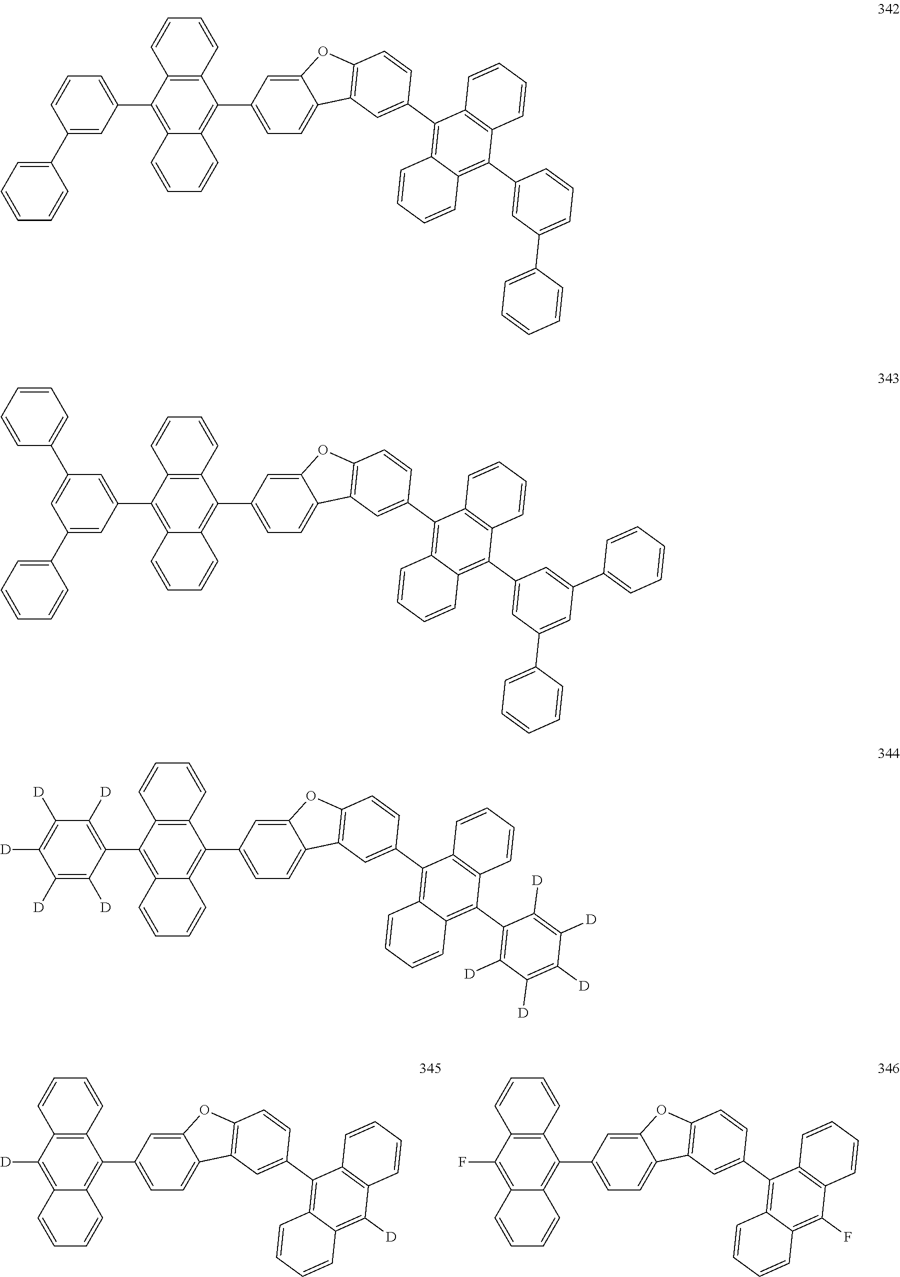

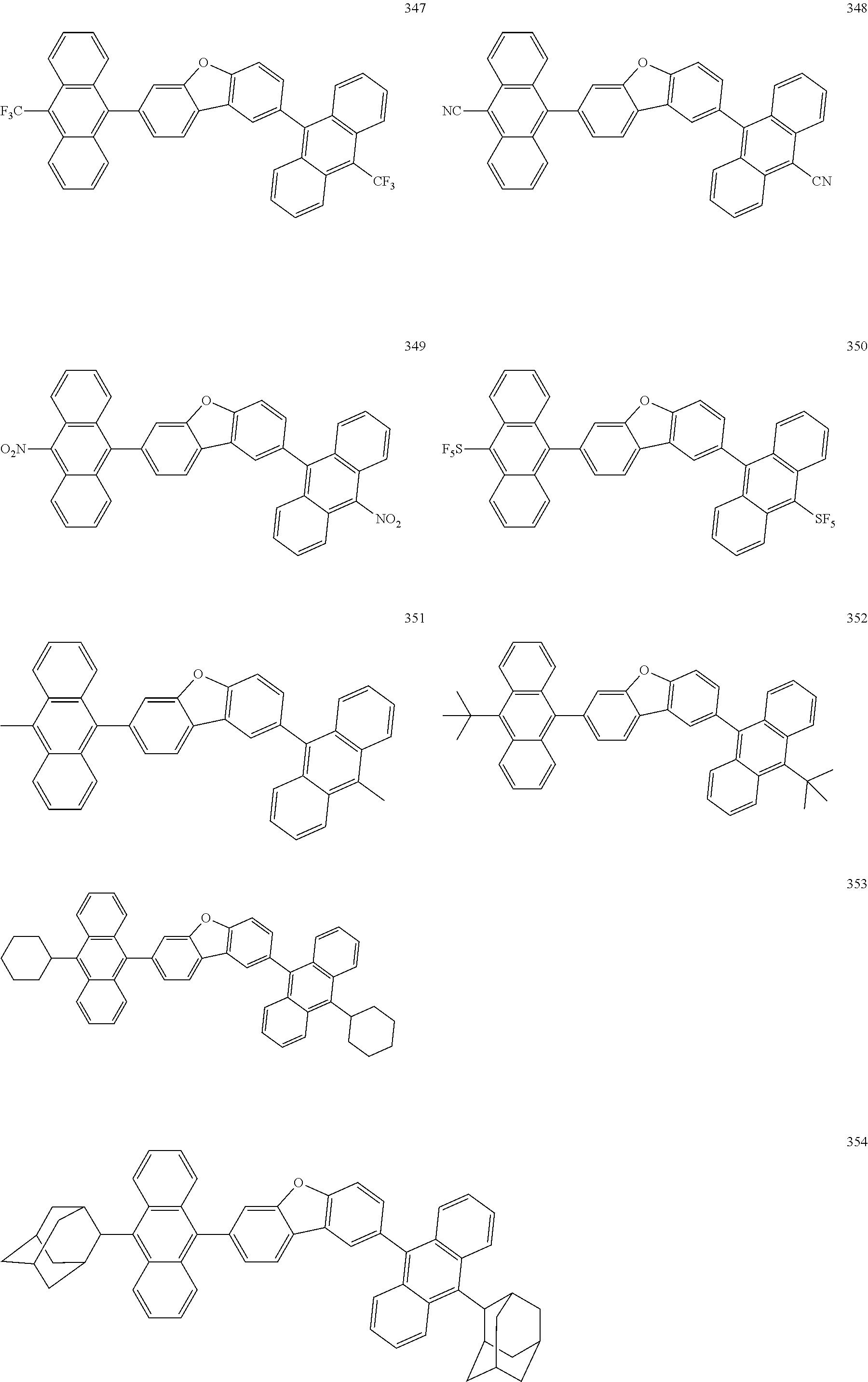

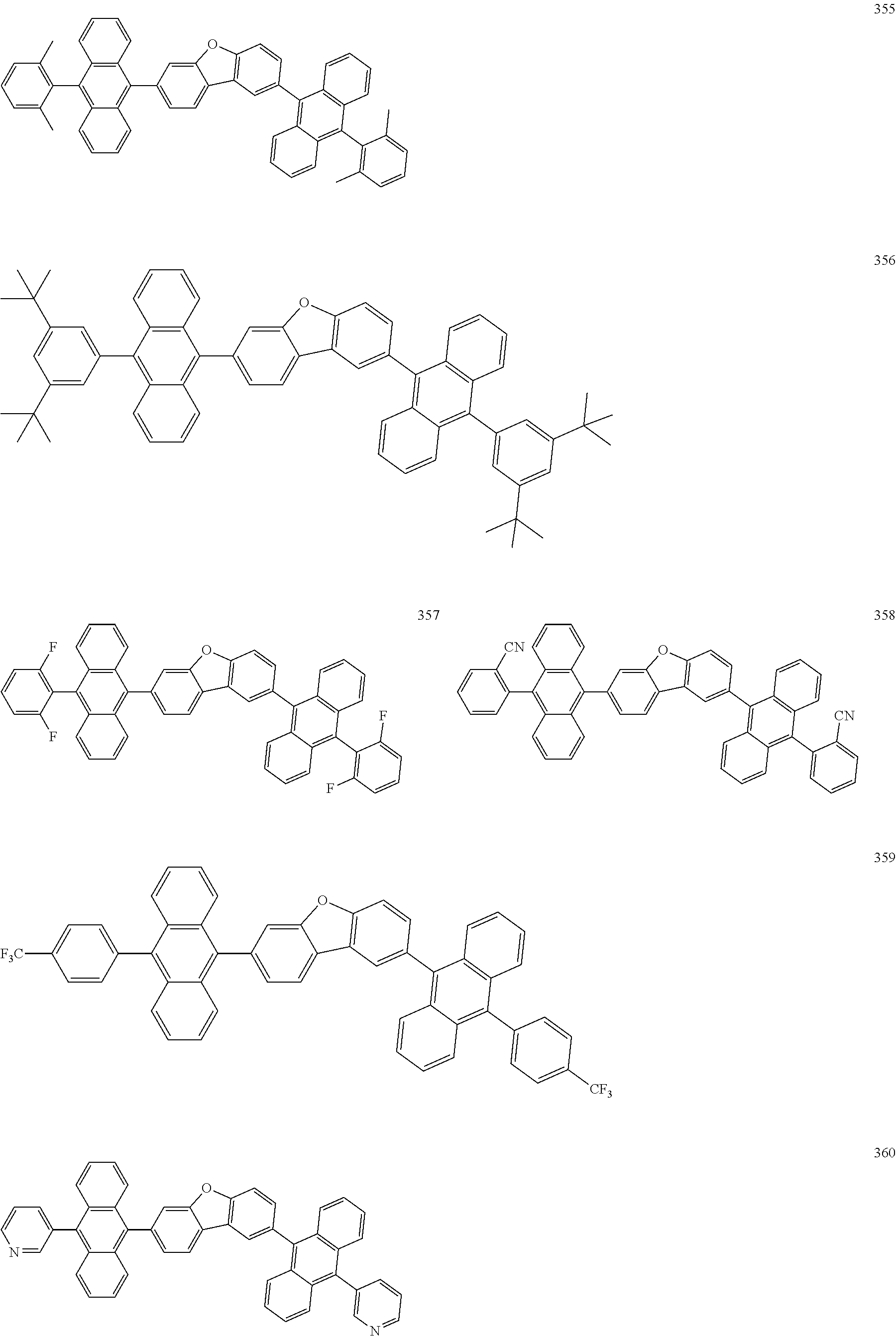

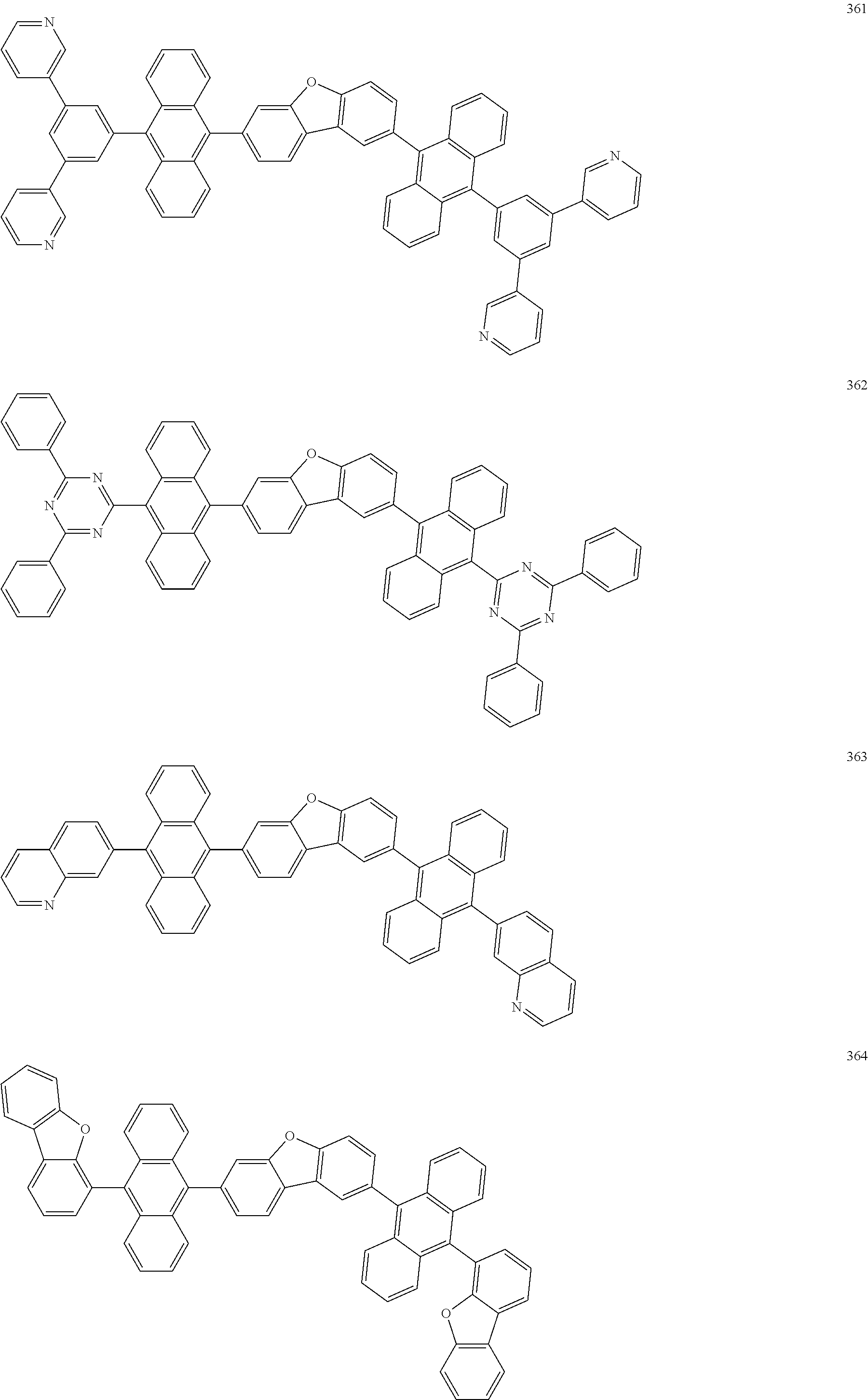 [SMILES: c1cncc(-c2cc(-c3cccnc3)cc(-c3c4ccccc4c(-c4ccc5c(c4)oc4ccc(-c6c7ccccc7c(-c7cc(-c8cccnc8)cc(-c8cccnc8)c7)c7ccccc67)cc45)c4ccccc34)c2)c1 ; c1ccc(-c2nc(-c3ccccc3)nc(-c3c4ccccc4c(-c4ccc5c(c4)oc4ccc(-c6c7ccccc7c(-c7nc(-c8ccccc8)nc(-c8ccccc8)n7)c7ccccc67)cc45)c4ccccc34)n2)cc1 ; c1cnc2cc(-c3c4ccccc4c(-c4ccc5c(c4)oc4ccc(-c6c7ccccc7c(-c7ccc8cccnc8c7)c7ccccc67)cc45)c4ccccc34)ccc2c1 ; c1ccc2c(c1)oc1c(-c3c4ccccc4c(-c4ccc5c(c4)oc4ccc(-c6c7ccccc7c(-c7cccc8c7oc7ccccc78)c7ccccc67)cc45)c4ccccc34)cccc12]

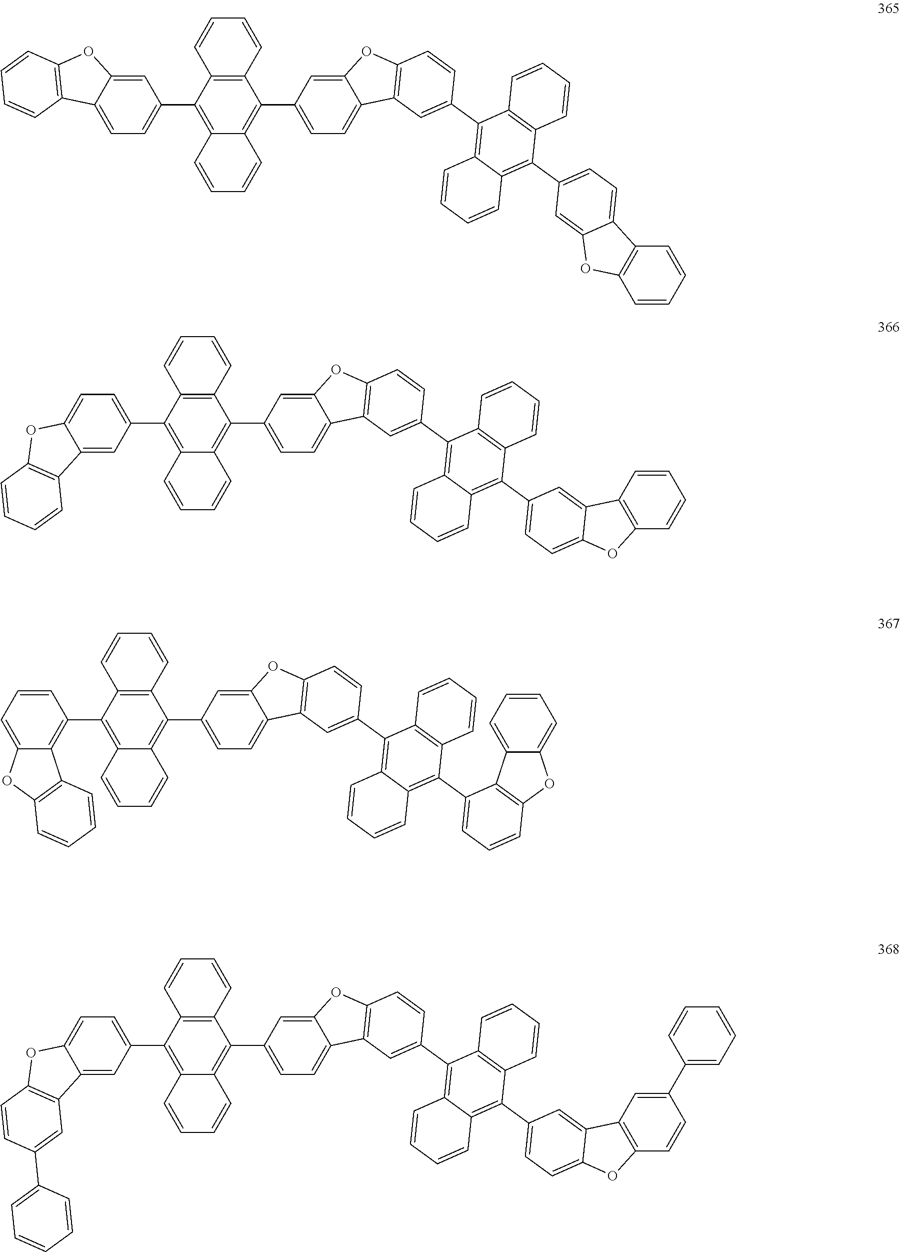 [SMILES: c1ccc2c(c1)oc1cc(-c3c4ccccc4c(-c4ccc5c(c4)oc4ccc(-c6c7ccccc7c(-c7ccc8c(c7)oc7ccccc78)c7ccccc67)cc45)c4ccccc34)ccc12 ; c1ccc2c(c1)oc1ccc(-c3c4ccccc4c(-c4ccc5c(c4)oc4ccc(-c6c7ccccc7c(-c7ccc8oc9ccccc9c8c7)c7ccccc67)cc45)c4ccccc34)cc12 ; c1ccc2c(c1)oc1cccc(-c3c4ccccc4c(-c4ccc5c(c4)oc4ccc(-c6c7ccccc7c(-c7cccc8oc9ccccc9c78)c7ccccc67)cc45)c4ccccc34)c12 ; c1ccc(-c2ccc3oc4ccc(-c5c6ccccc6c(-c6ccc7c(c6)oc6ccc(-c8c9ccccc9c(-c9ccc%10oc%11ccc(-c%12ccccc%12)cc%11c%10c9)c9ccccc89)cc67)c6ccccc56)cc4c3c2)cc1]

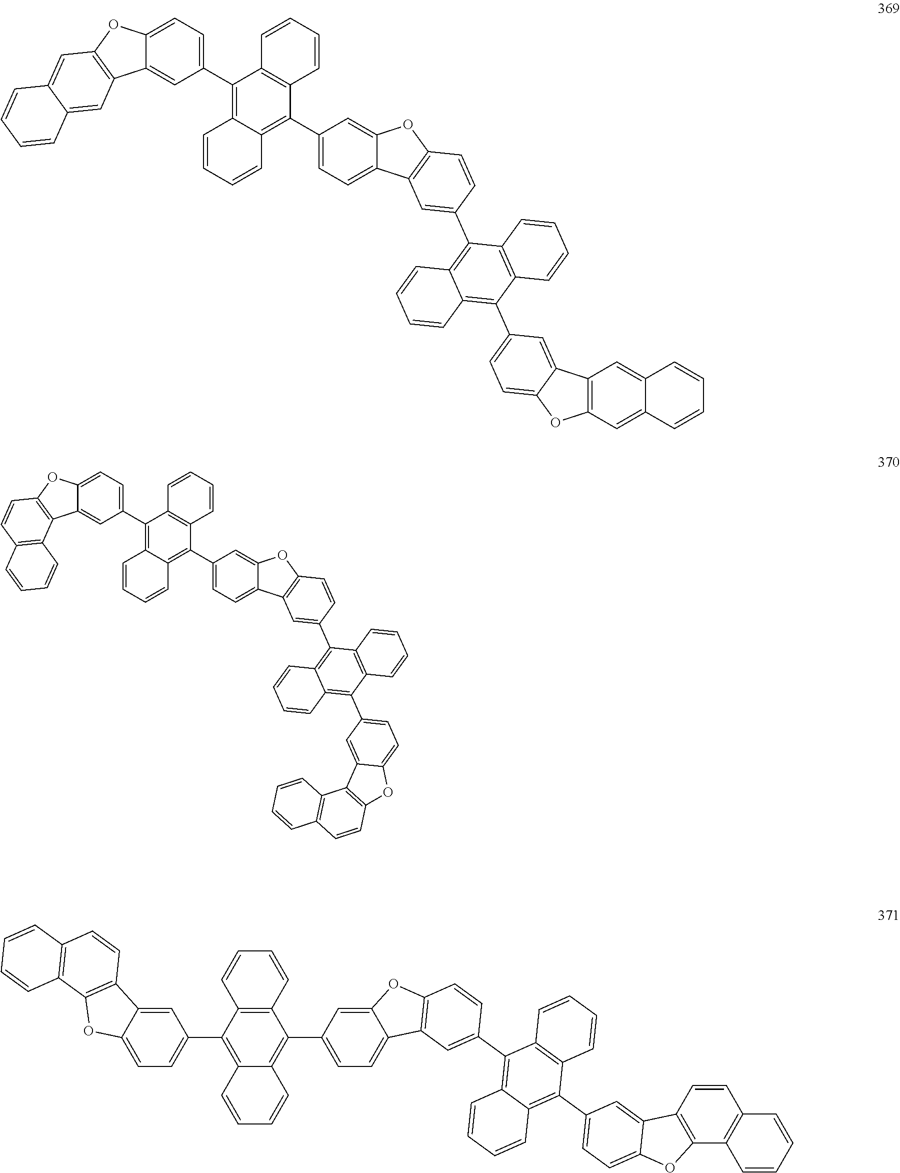 [SMILES: c1ccc2cc3c(cc2c1)oc1ccc(-c2c4ccccc4c(-c4ccc5c(c4)oc4ccc(-c6c7ccccc7c(-c7ccc8oc9cc%10ccccc%10cc9c8c7)c7ccccc67)cc45)c4ccccc24)cc13 ; c1ccc2c(c1)ccc1oc3ccc(-c4c5ccccc5c(-c5ccc6c(c5)oc5ccc(-c7c8ccccc8c(-c8ccc9oc%10ccc%11ccccc%11c%10c9c8)c8ccccc78)cc56)c5ccccc45)cc3c12 ; c1ccc2c(c1)ccc1c3cc(-c4c5ccccc5c(-c5ccc6c(c5)oc5ccc(-c7c8ccccc8c(-c8ccc9oc%10c%11ccccc%11ccc%10c9c8)c8ccccc78)cc56)c5ccccc45)ccc3oc21]

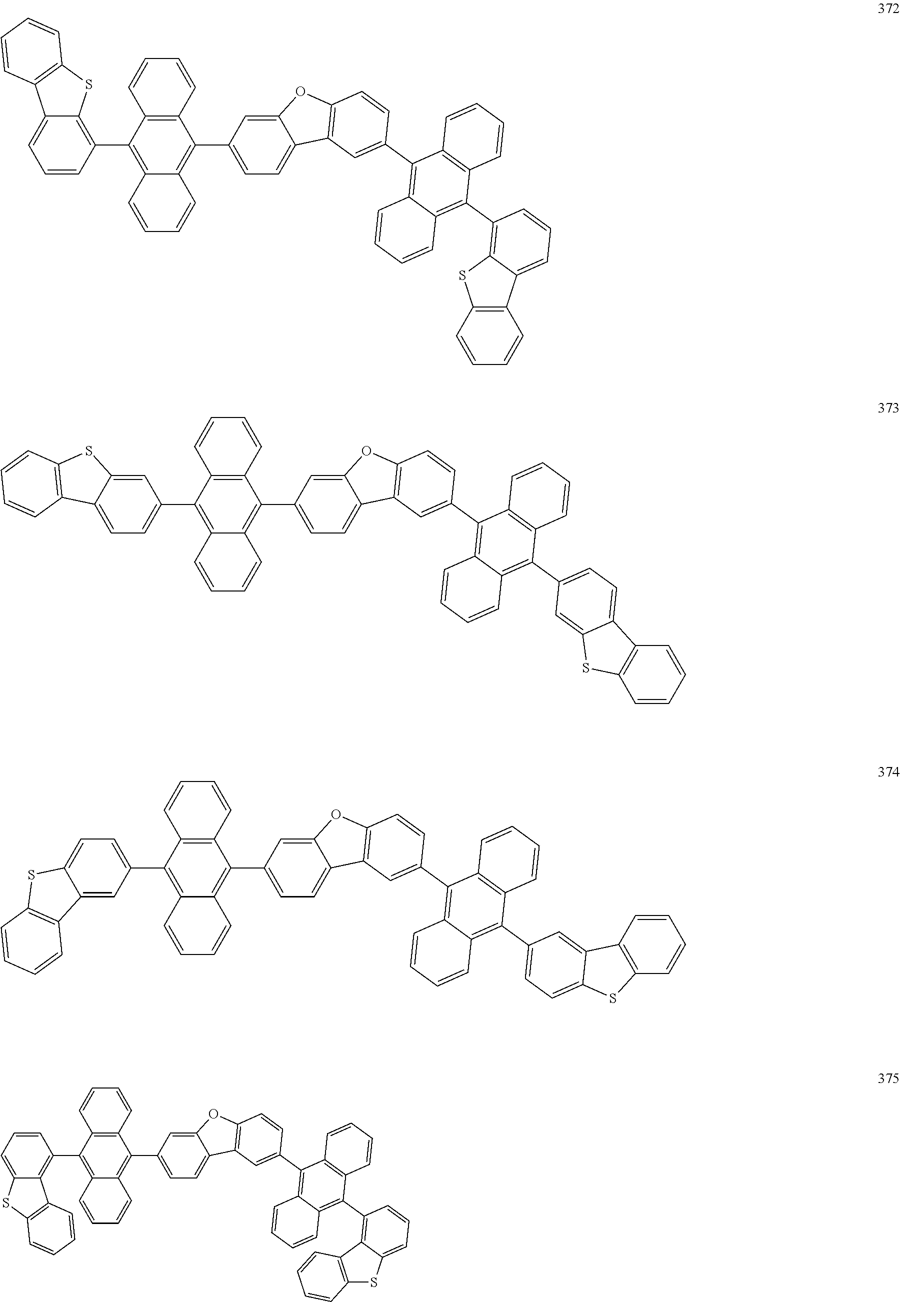 [SMILES: c1ccc2c(c1)sc1c(-c3c4ccccc4c(-c4ccc5c(c4)oc4ccc(-c6c7ccccc7c(-c7cccc8c7sc7ccccc78)c7ccccc67)cc45)c4ccccc34)cccc12 ; c1ccc2c(c1)sc1cc(-c3c4ccccc4c(-c4ccc5c(c4)oc4ccc(-c6c7ccccc7c(-c7ccc8c(c7)sc7ccccc78)c7ccccc67)cc45)c4ccccc34)ccc12 ; c1ccc2c(c1)sc1ccc(-c3c4ccccc4c(-c4ccc5c(c4)oc4ccc(-c6c7ccccc7c(-c7ccc8sc9ccccc9c8c7)c7ccccc67)cc45)c4ccccc34)cc12 ; c1ccc2c(c1)sc1cccc(-c3c4ccccc4c(-c4ccc5c(c4)oc4ccc(-c6c7ccccc7c(-c7cccc8sc9ccccc9c78)c7ccccc67)cc45)c4ccccc34)c12]

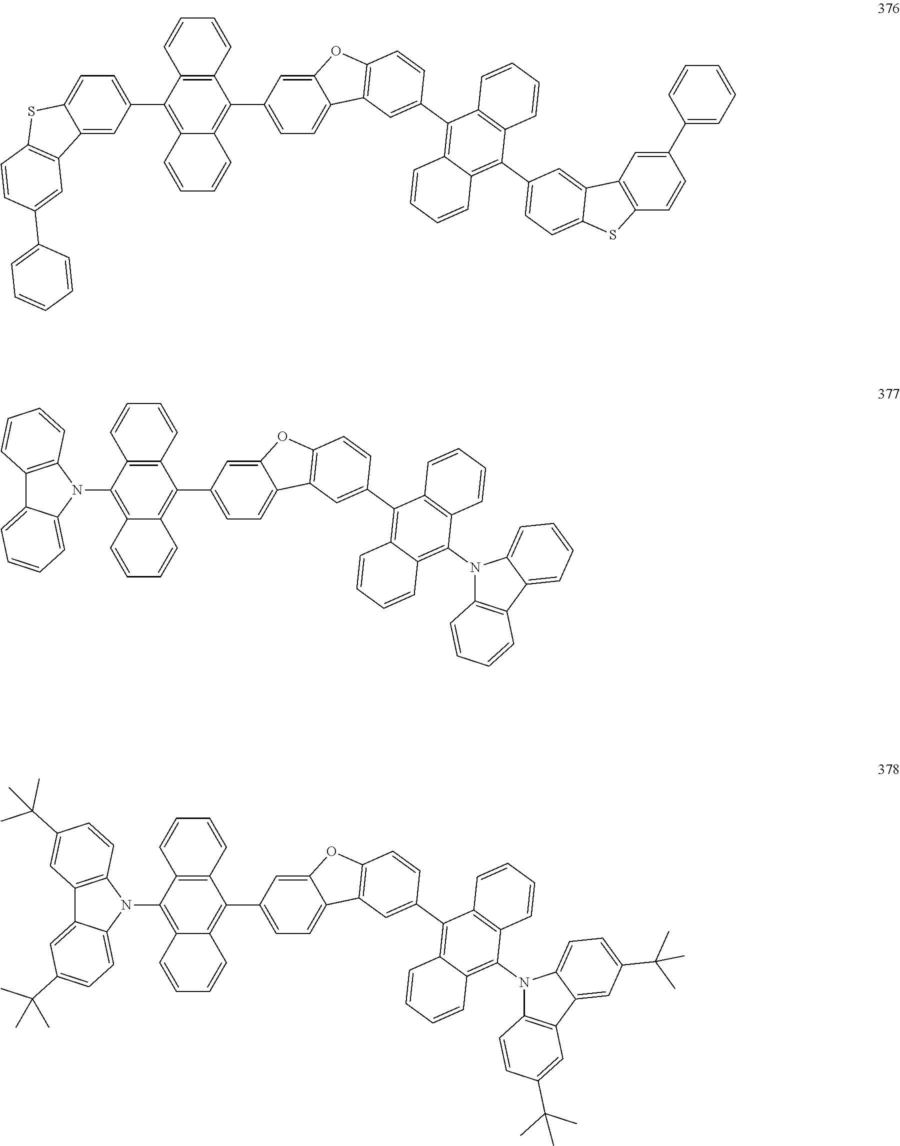 [SMILES: c1ccc(-c2ccc3sc4ccc(-c5c6ccccc6c(-c6ccc7c(c6)oc6ccc(-c8c9ccccc9c(-c9ccc%10sc%11ccc(-c%12ccccc%12)cc%11c%10c9)c9ccccc89)cc67)c6ccccc56)cc4c3c2)cc1 ; c1ccc2c(-n3c4ccccc4c4ccccc43)c3ccccc3c(-c3ccc4c(c3)oc3ccc(-c5c6ccccc6c(-n6c7ccccc7c7ccccc76)c6ccccc56)cc34)c2c1 ; CC(C)(C)c1ccc2c(c1)c1cc(C(C)(C)C)ccc1n2-c1c2ccccc2c(-c2ccc3c(c2)oc2ccc(-c4c5ccccc5c(-n5c6ccc(C(C)(C)C)cc6c6cc(C(C)(C)C)ccc65)c5ccccc45)cc23)c2ccccc12]

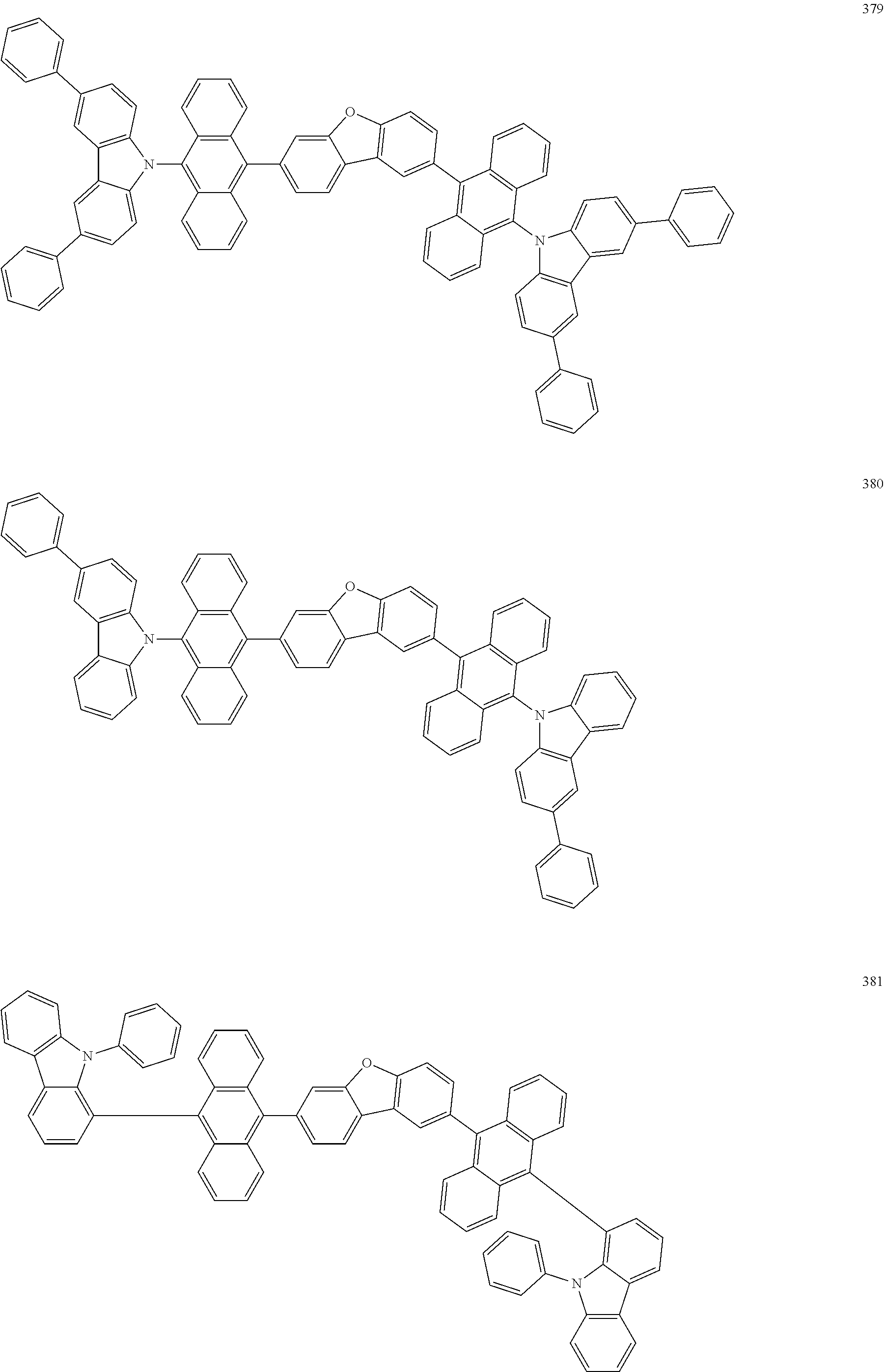 [SMILES: c1ccc(-c2ccc3c(c2)c2cc(-c4ccccc4)ccc2n3-c2c3ccccc3c(-c3ccc4c(c3)oc3ccc(-c5c6ccccc6c(-n6c7ccc(-c8ccccc8)cc7c7cc(-c8ccccc8)ccc76)c6ccccc56)cc34)c3ccccc23)cc1 ; c1ccc(-c2ccc3c(c2)c2ccccc2n3-c2c3ccccc3c(-c3ccc4c(c3)oc3ccc(-c5c6ccccc6c(-n6c7ccccc7c7cc(-c8ccccc8)ccc76)c6ccccc56)cc34)c3ccccc23)cc1 ; c1ccc(-n2c3ccccc3c3cccc(-c4c5ccccc5c(-c5ccc6c(c5)oc5ccc(-c7c8ccccc8c(-c8cccc9c%10ccccc%10n(-c%10ccccc%10)c89)c8ccccc78)cc56)c5ccccc45)c32)cc1]

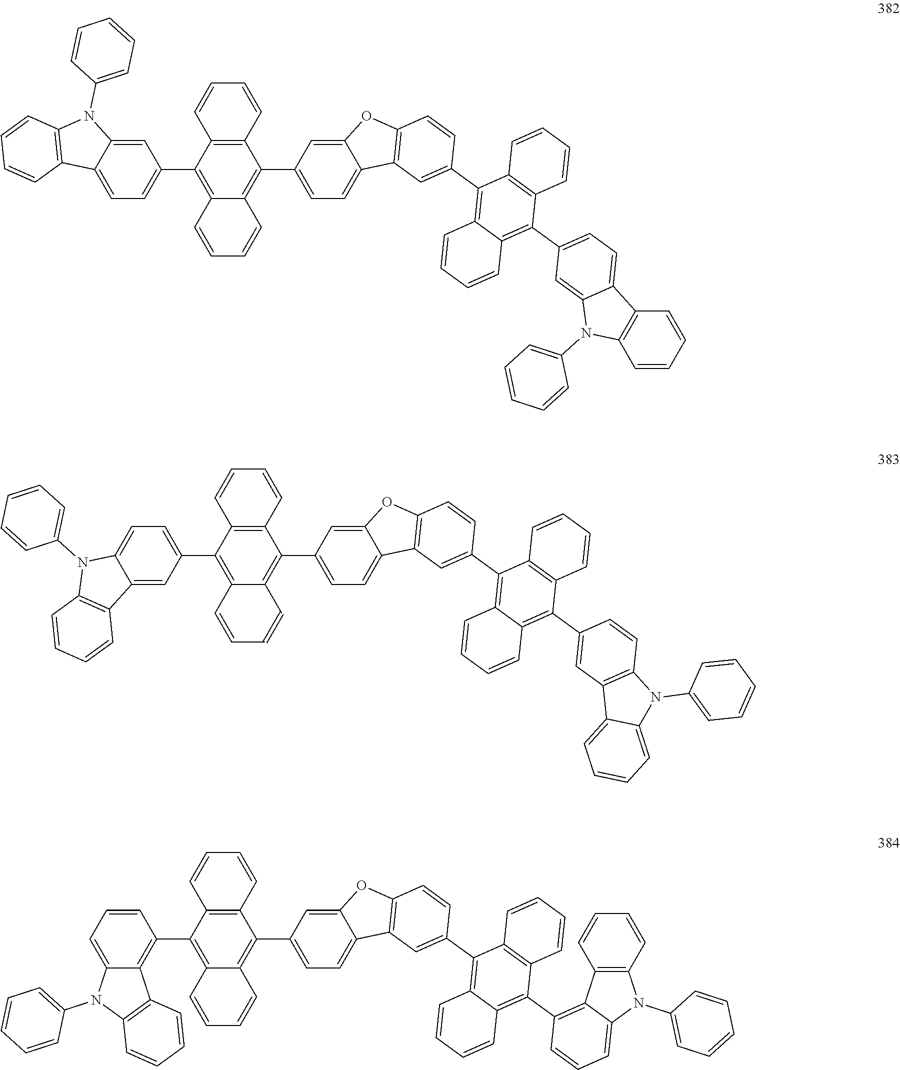 [SMILES: c1ccc(-n2c3ccccc3c3ccc(-c4c5ccccc5c(-c5ccc6c(c5)oc5ccc(-c7c8ccccc8c(-c8ccc9c%10ccccc%10n(-c%10ccccc%10)c9c8)c8ccccc78)cc56)c5ccccc45)cc32)cc1 ; c1ccc(-n2c3ccccc3c3cc(-c4c5ccccc5c(-c5ccc6c(c5)oc5ccc(-c7c8ccccc8c(-c8ccc9c(c8)c8ccccc8n9-c8ccccc8)c8ccccc78)cc56)c5ccccc45)ccc32)cc1 ; c1ccc(-n2c3ccccc3c3c(-c4c5ccccc5c(-c5ccc6c(c5)oc5ccc(-c7c8ccccc8c(-c8cccc9c8c8ccccc8n9-c8ccccc8)c8ccccc78)cc56)c5ccccc45)cccc32)cc1]

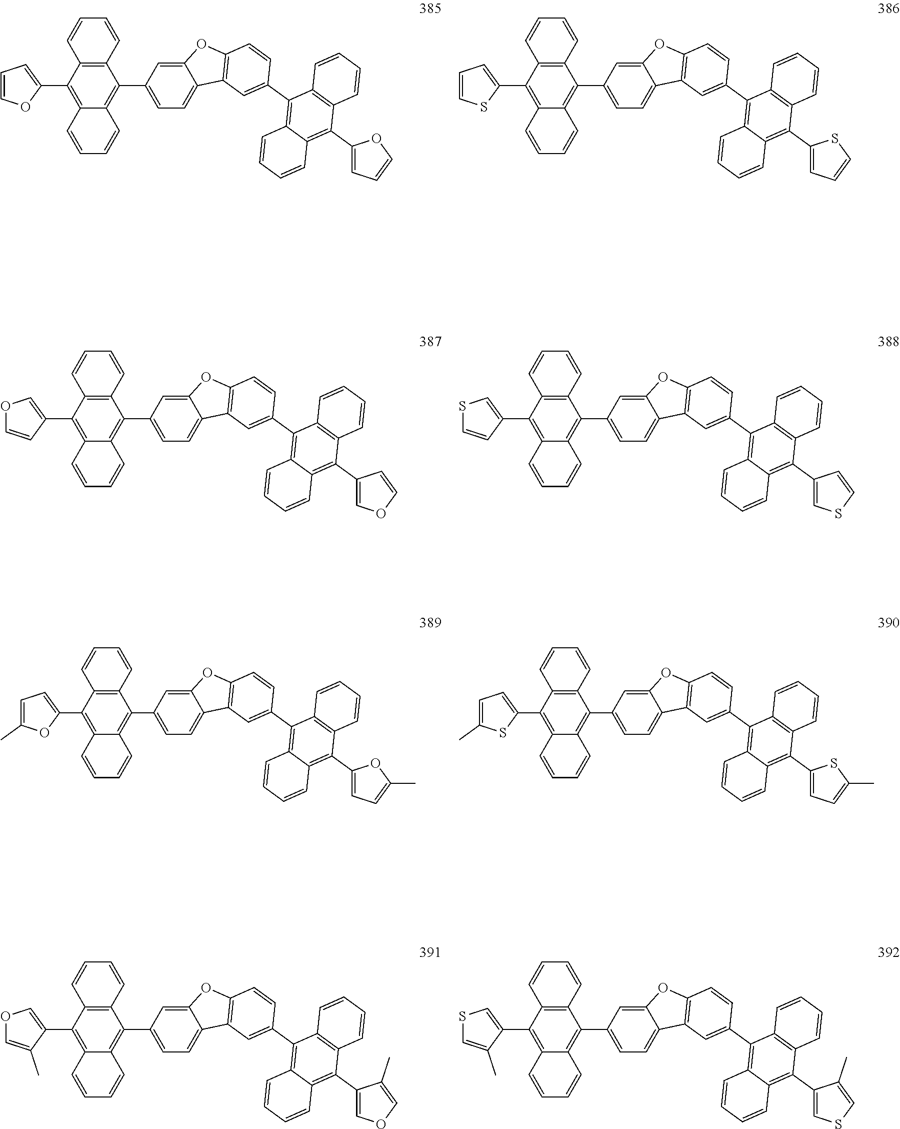

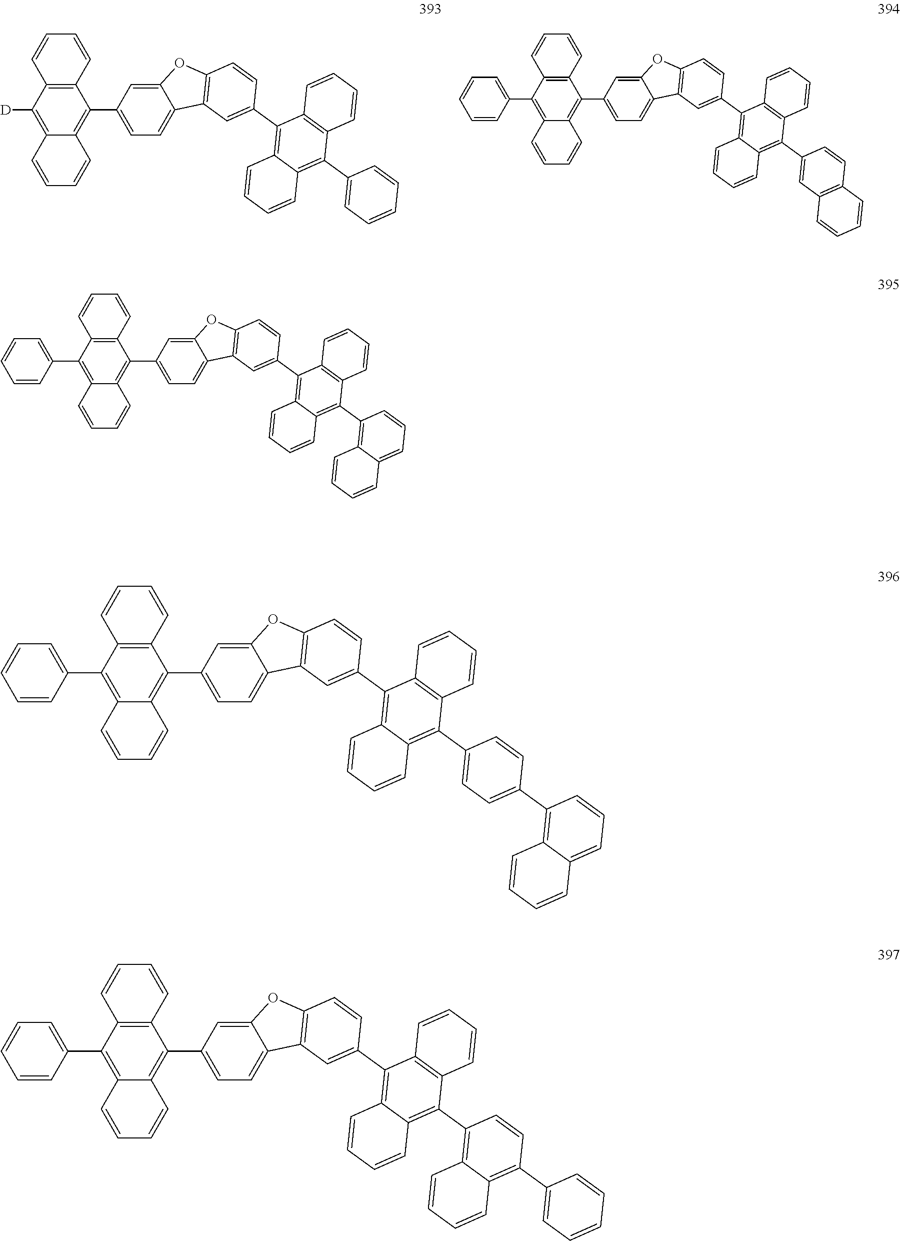 [SMILES: [2H]c1c2ccccc2c(-c2ccc3c(c2)oc2ccc(-c4c5ccccc5c(-c5ccccc5)c5ccccc45)cc23)c2ccccc12 ; c1ccc(-c2c3ccccc3c(-c3ccc4c(c3)oc3ccc(-c5c6ccccc6c(-c6ccc7ccccc7c6)c6ccccc56)cc34)c3ccccc23)cc1 ; c1ccc(-c2c3ccccc3c(-c3ccc4c(c3)oc3ccc(-c5c6ccccc6c(-c6cccc7ccccc67)c6ccccc56)cc34)c3ccccc23)cc1 ; c1ccc(-c2c3ccccc3c(-c3ccc4c(c3)oc3ccc(-c5c6ccccc6c(-c6ccc(-c7cccc8ccccc78)cc6)c6ccccc56)cc34)c3ccccc23)cc1 ; c1ccc(-c2ccc(-c3c4ccccc4c(-c4ccc5oc6cc(-c7c8ccccc8c(-c8ccccc8)c8ccccc78)ccc6c5c4)c4ccccc34)c3ccccc23)cc1]

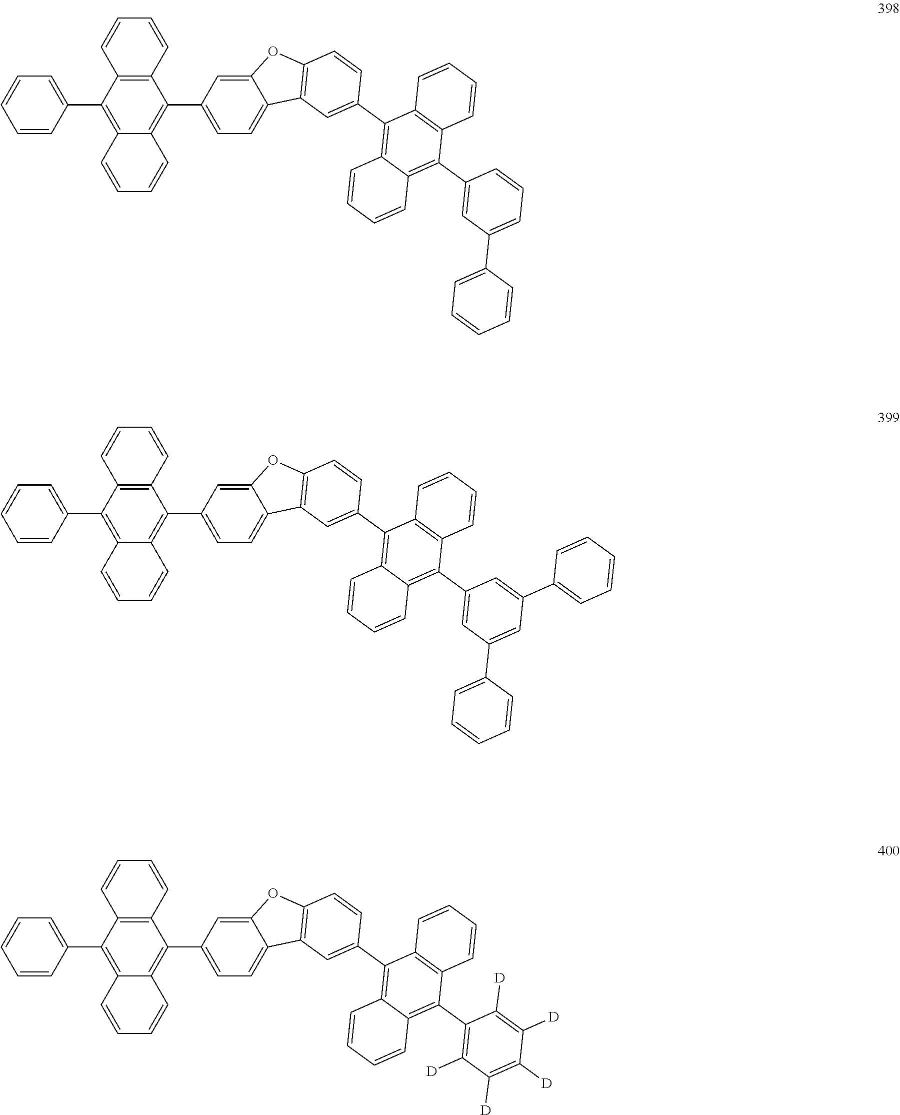 [SMILES: c1ccc(-c2cccc(-c3c4ccccc4c(-c4ccc5oc6cc(-c7c8ccccc8c(-c8ccccc8)c8ccccc78)ccc6c5c4)c4ccccc34)c2)cc1 ; c1ccc(-c2cc(-c3ccccc3)cc(-c3c4ccccc4c(-c4ccc5oc6cc(-c7c8ccccc8c(-c8ccccc8)c8ccccc78)ccc6c5c4)c4ccccc34)c2)cc1 ; [2H]c1c([2H])c([2H])c(-c2c3ccccc3c(-c3ccc4oc5cc(-c6c7ccccc7c(-c7ccccc7)c7ccccc67)ccc5c4c3)c3ccccc23)c([2H])c1[2H]]

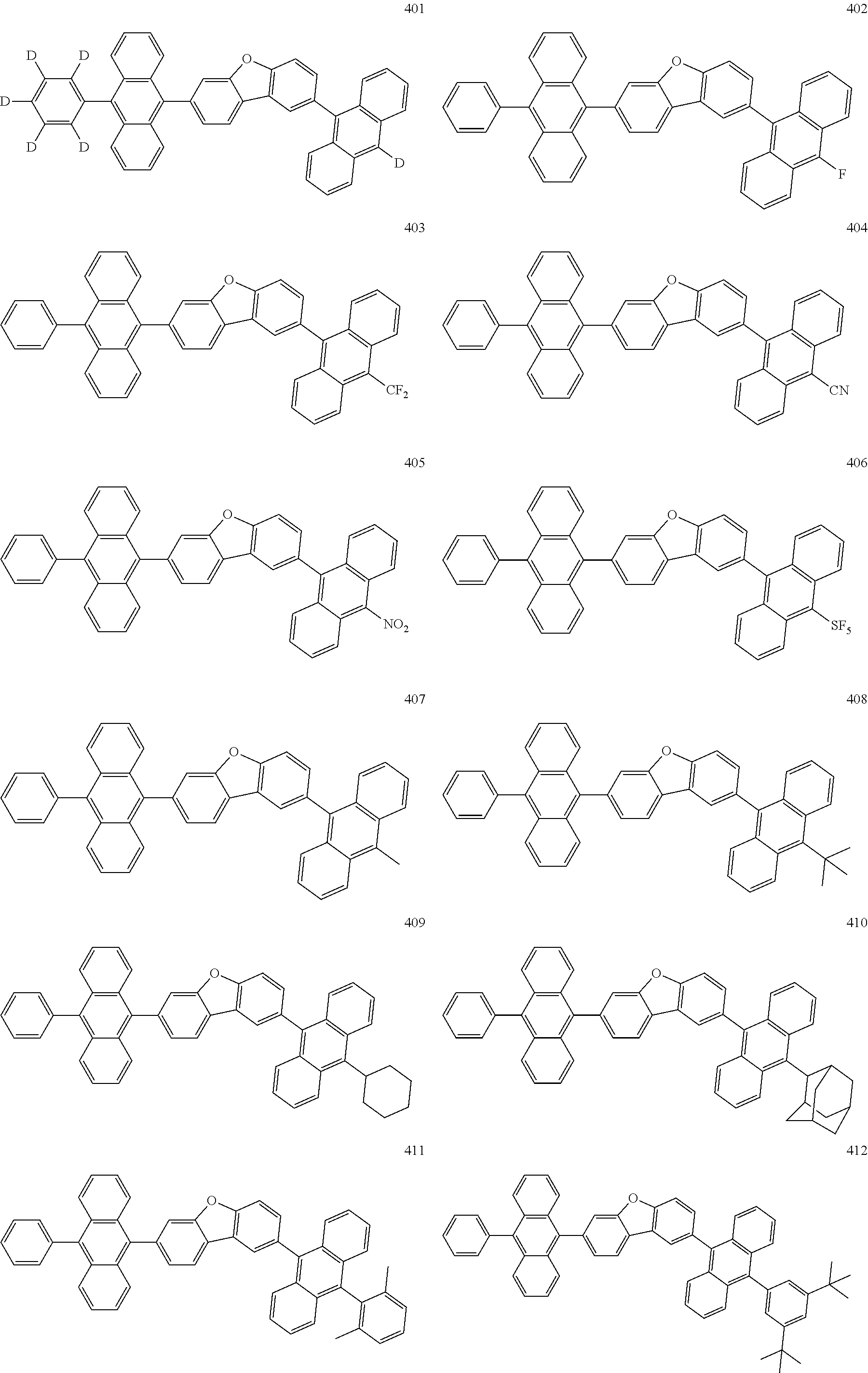

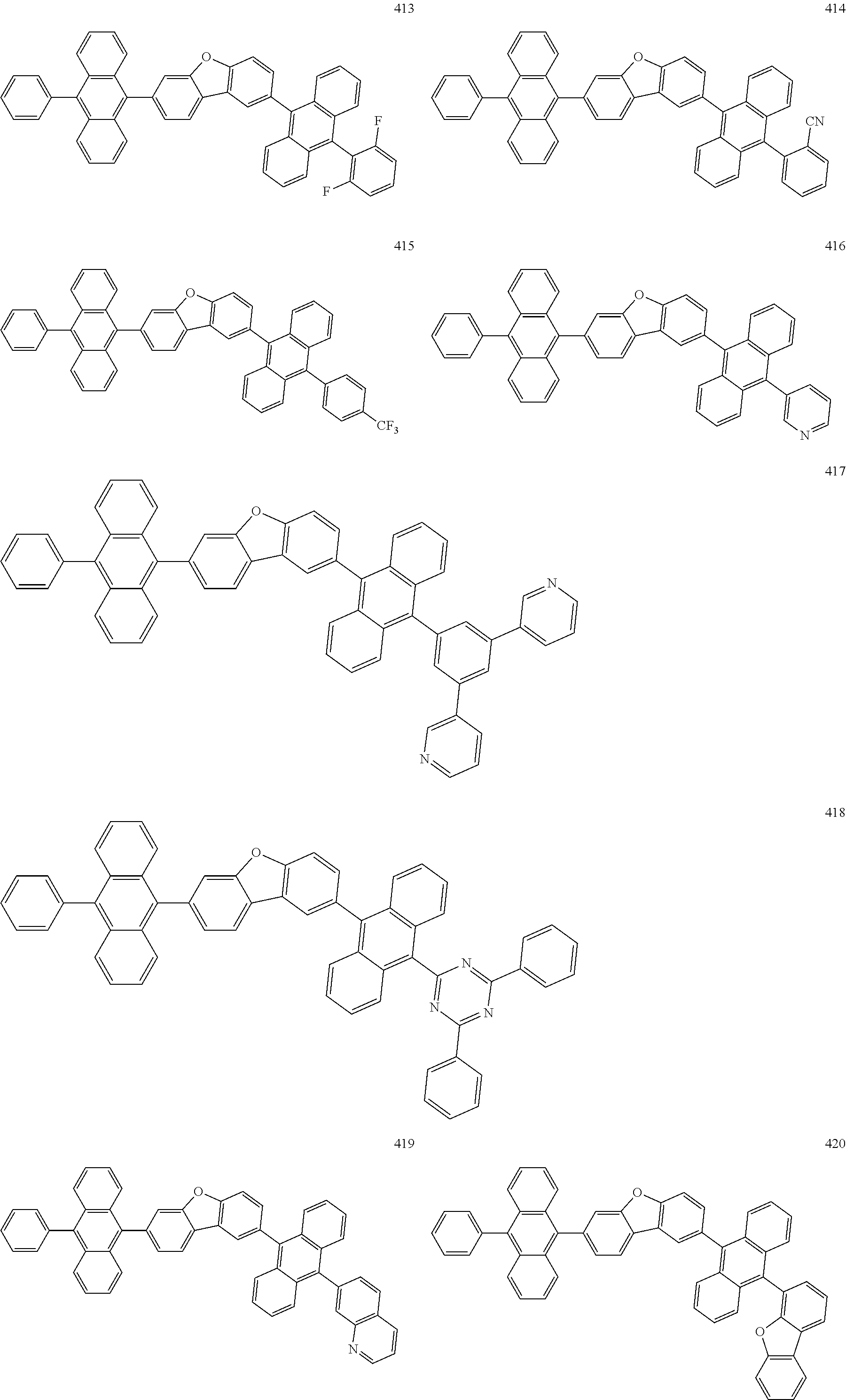

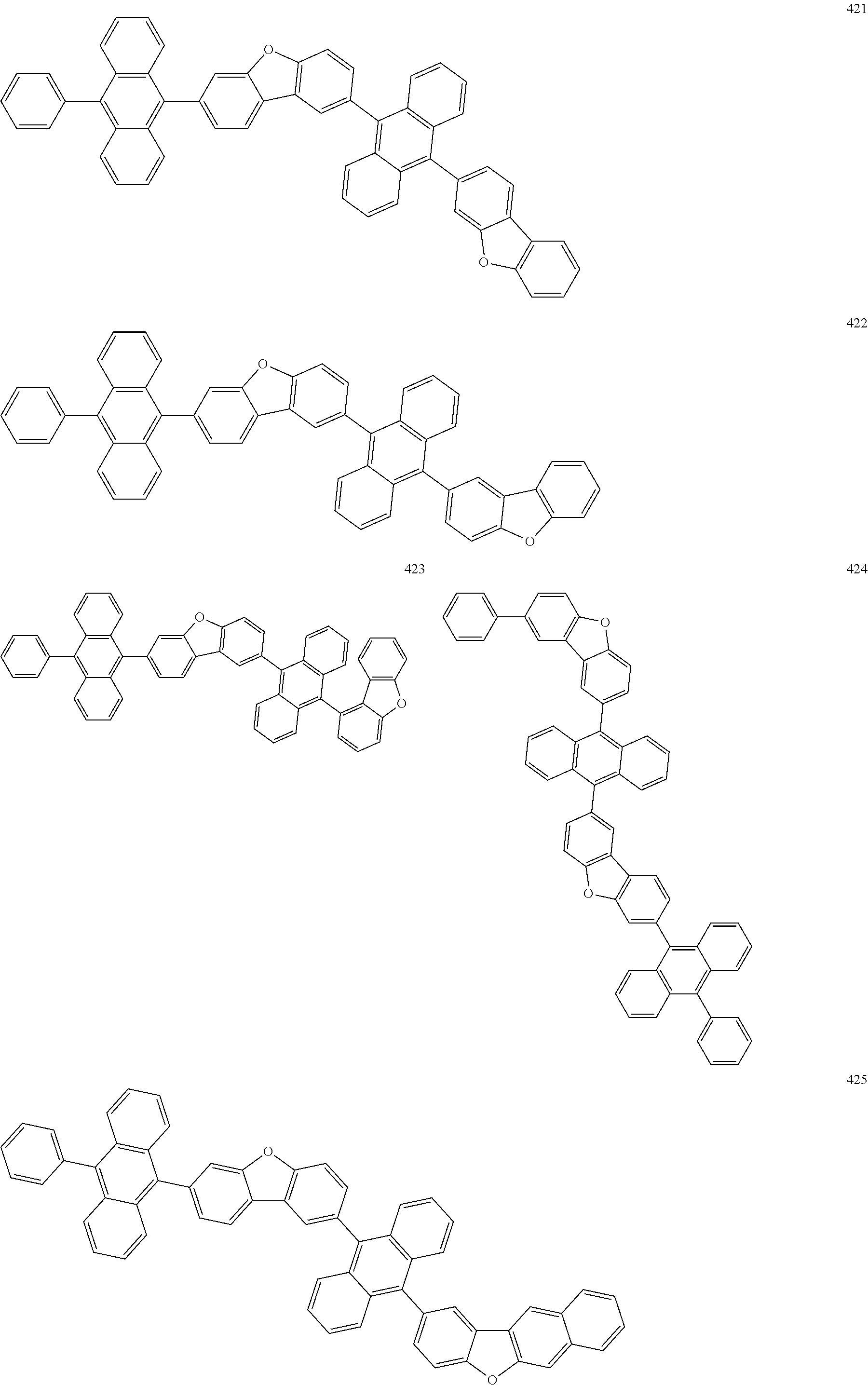

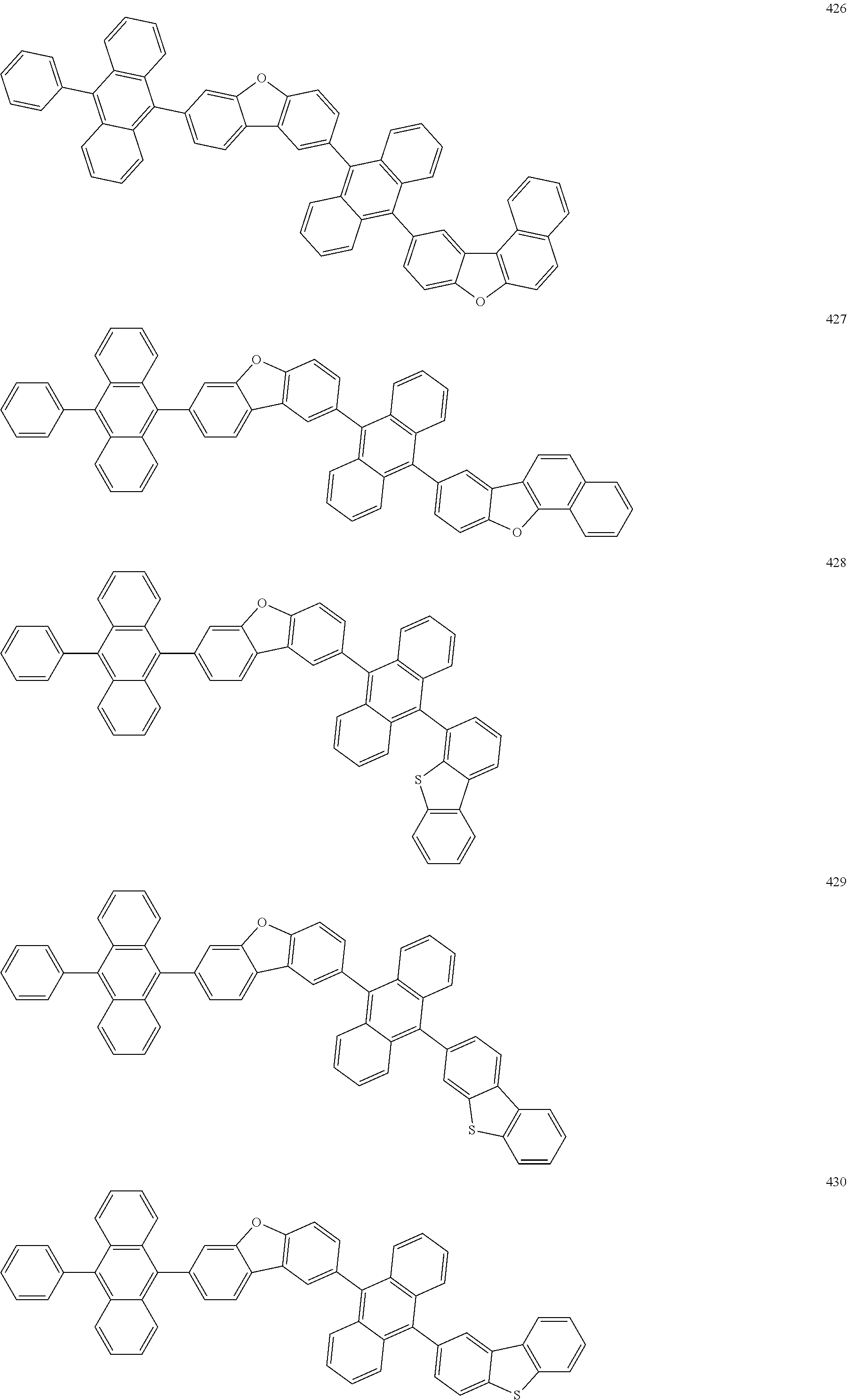

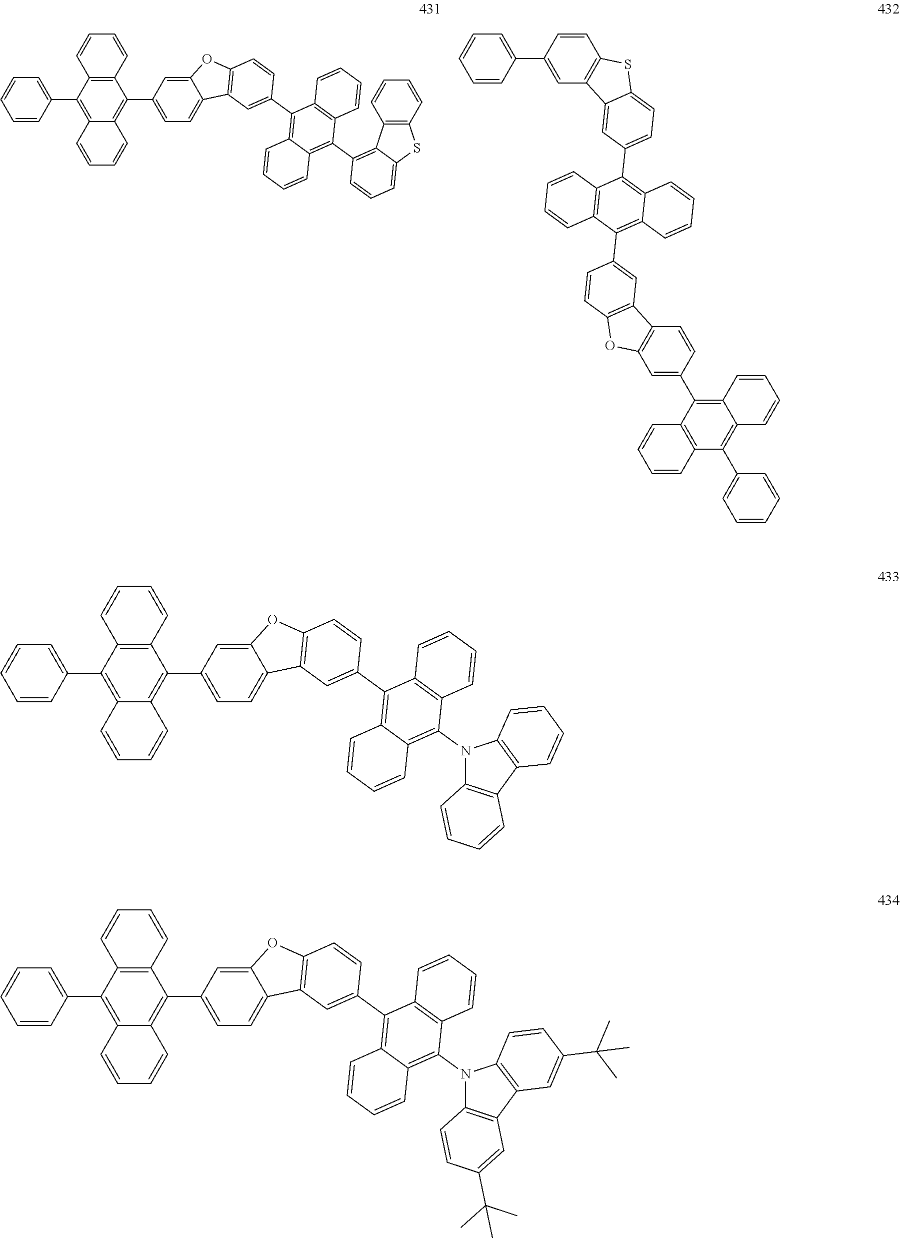 [SMILES: c1ccc(-c2c3ccccc3c(-c3ccc4c(c3)oc3ccc(-c5c6ccccc6c(-c6cccc7sc8ccccc8c67)c6ccccc56)cc34)c3ccccc23)cc1 ; c1ccc(-c2ccc3sc4ccc(-c5c6ccccc6c(-c6ccc7oc8cc(-c9c%10ccccc%10c(-c%10ccccc%10)c%10ccccc9%10)ccc8c7c6)c6ccccc56)cc4c3c2)cc1 ; c1ccc(-c2c3ccccc3c(-c3ccc4c(c3)oc3ccc(-c5c6ccccc6c(-n6c7ccccc7c7ccccc76)c6ccccc56)cc34)c3ccccc23)cc1 ; CC(C)(C)c1ccc2c(c1)c1cc(C(C)(C)C)ccc1n2-c1c2ccccc2c(-c2ccc3oc4cc(-c5c6ccccc6c(-c6ccccc6)c6ccccc56)ccc4c3c2)c2ccccc12]

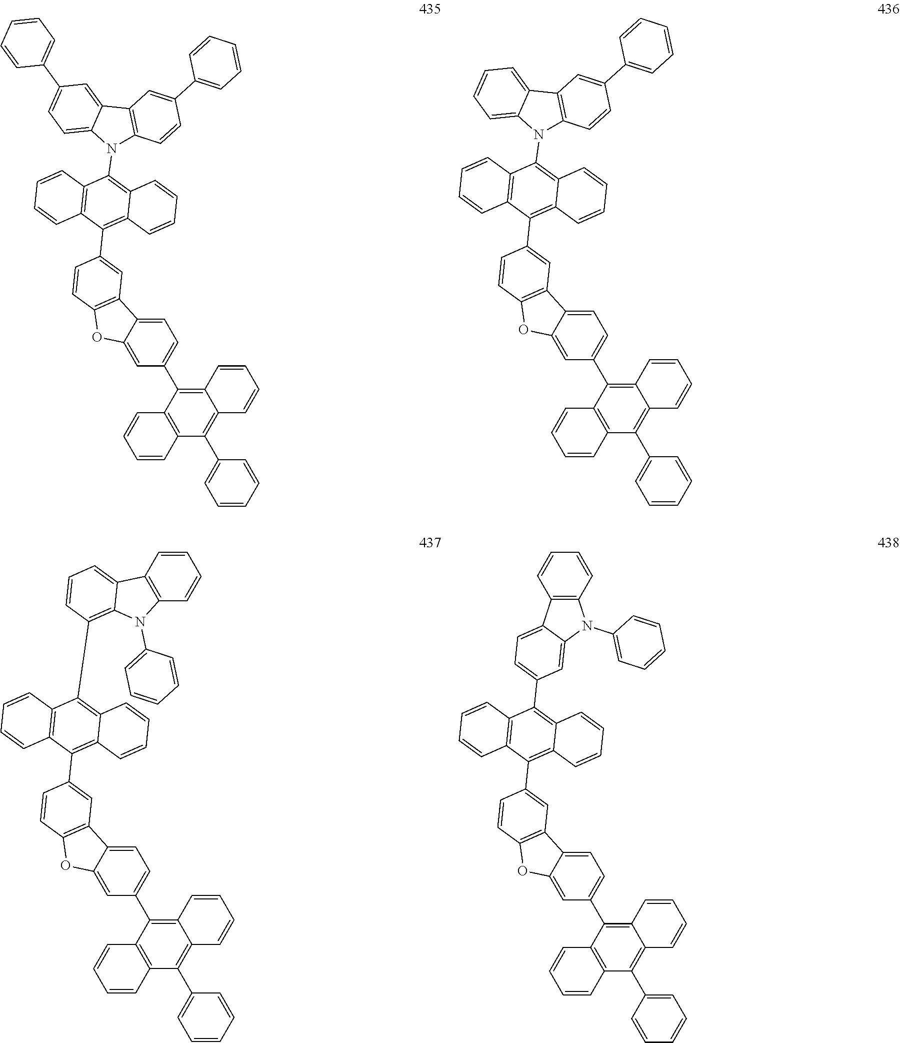 [SMILES: c1ccc(-c2ccc3c(c2)c2cc(-c4ccccc4)ccc2n3-c2c3ccccc3c(-c3ccc4oc5cc(-c6c7ccccc7c(-c7ccccc7)c7ccccc67)ccc5c4c3)c3ccccc23)cc1 ; c1ccc(-c2ccc3c(c2)c2ccccc2n3-c2c3ccccc3c(-c3ccc4oc5cc(-c6c7ccccc7c(-c7ccccc7)c7ccccc67)ccc5c4c3)c3ccccc23)cc1 ; c1ccc(-c2c3ccccc3c(-c3ccc4c(c3)oc3ccc(-c5c6ccccc6c(-c6cccc7c8ccccc8n(-c8ccccc8)c67)c6ccccc56)cc34)c3ccccc23)cc1 ; c1ccc(-c2c3ccccc3c(-c3ccc4c(c3)oc3ccc(-c5c6ccccc6c(-c6ccc7c8ccccc8n(-c8ccccc8)c7c6)c6ccccc56)cc34)c3ccccc23)cc1]

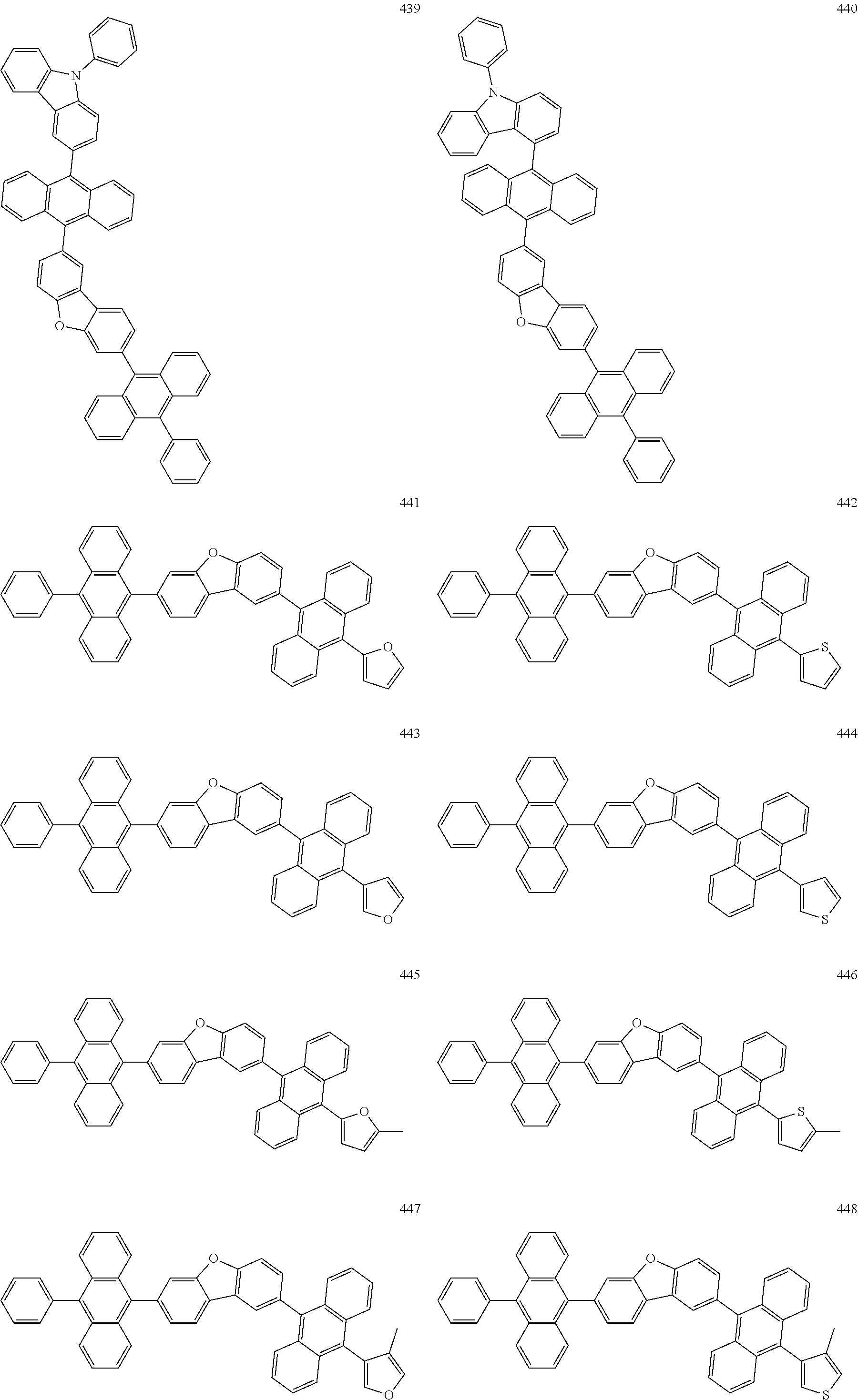

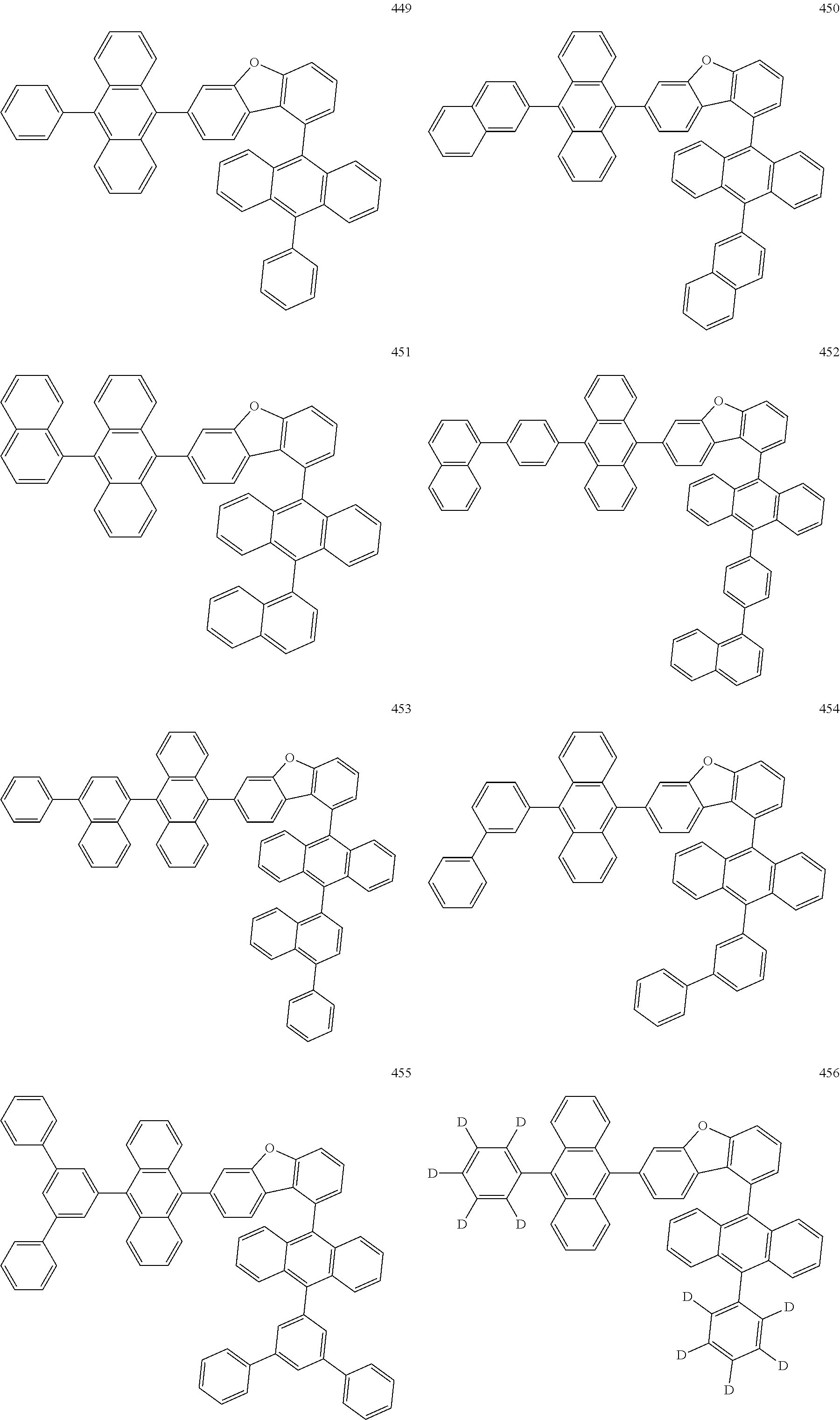

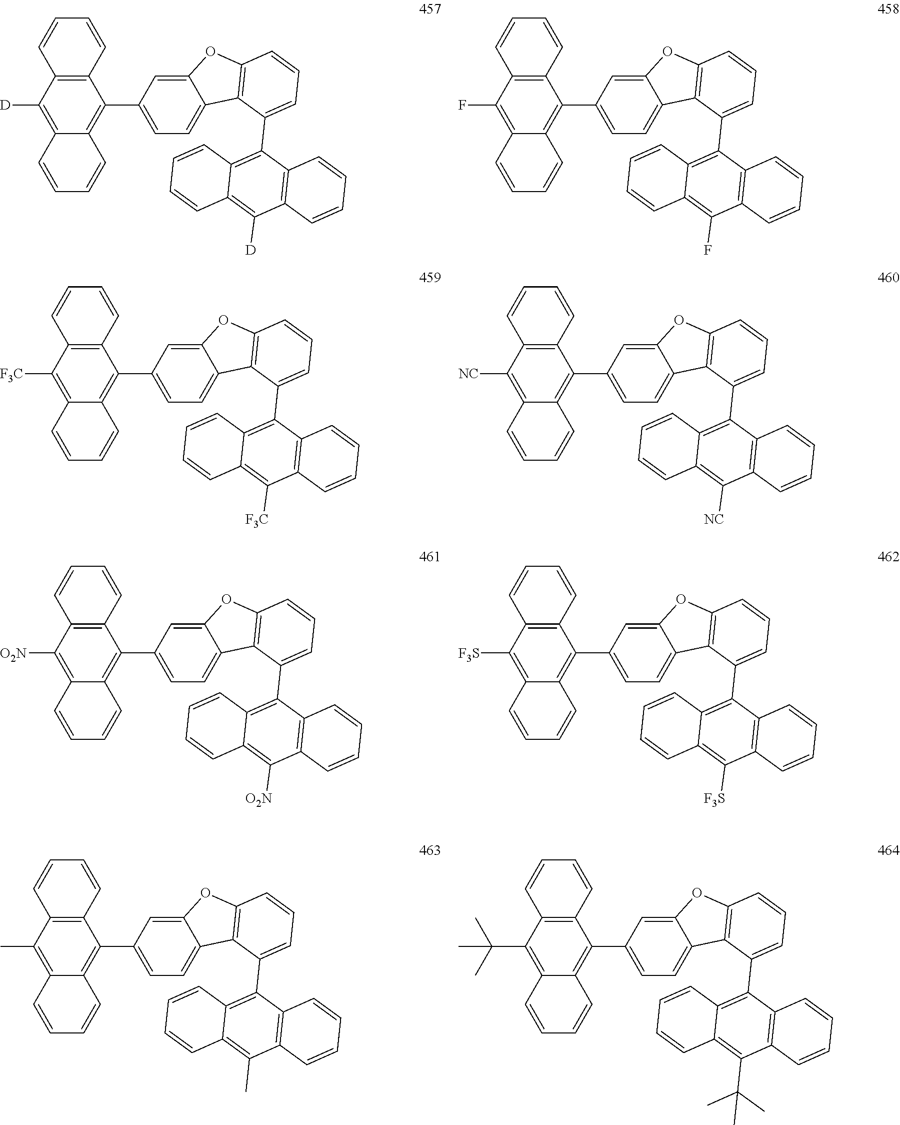

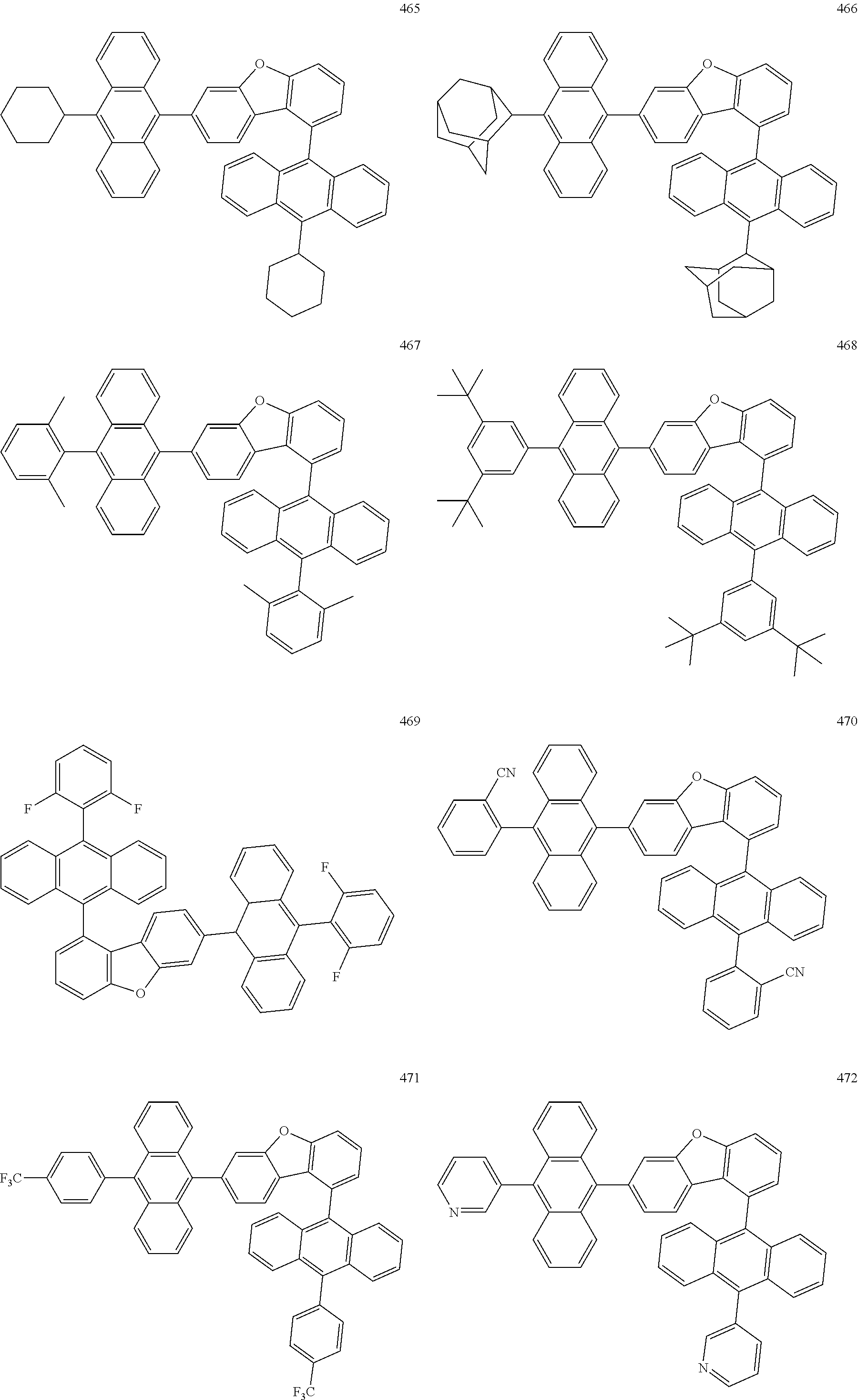

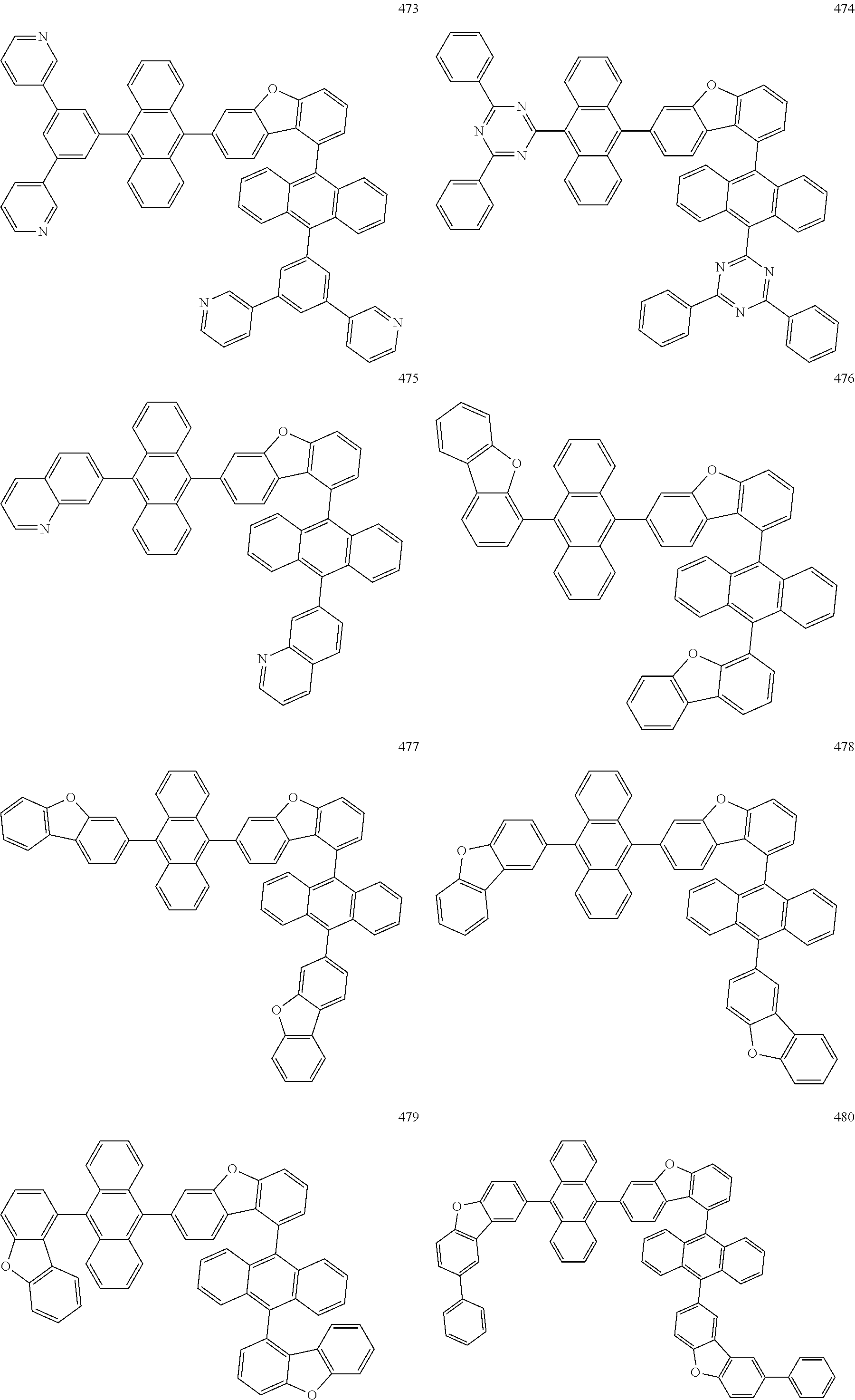

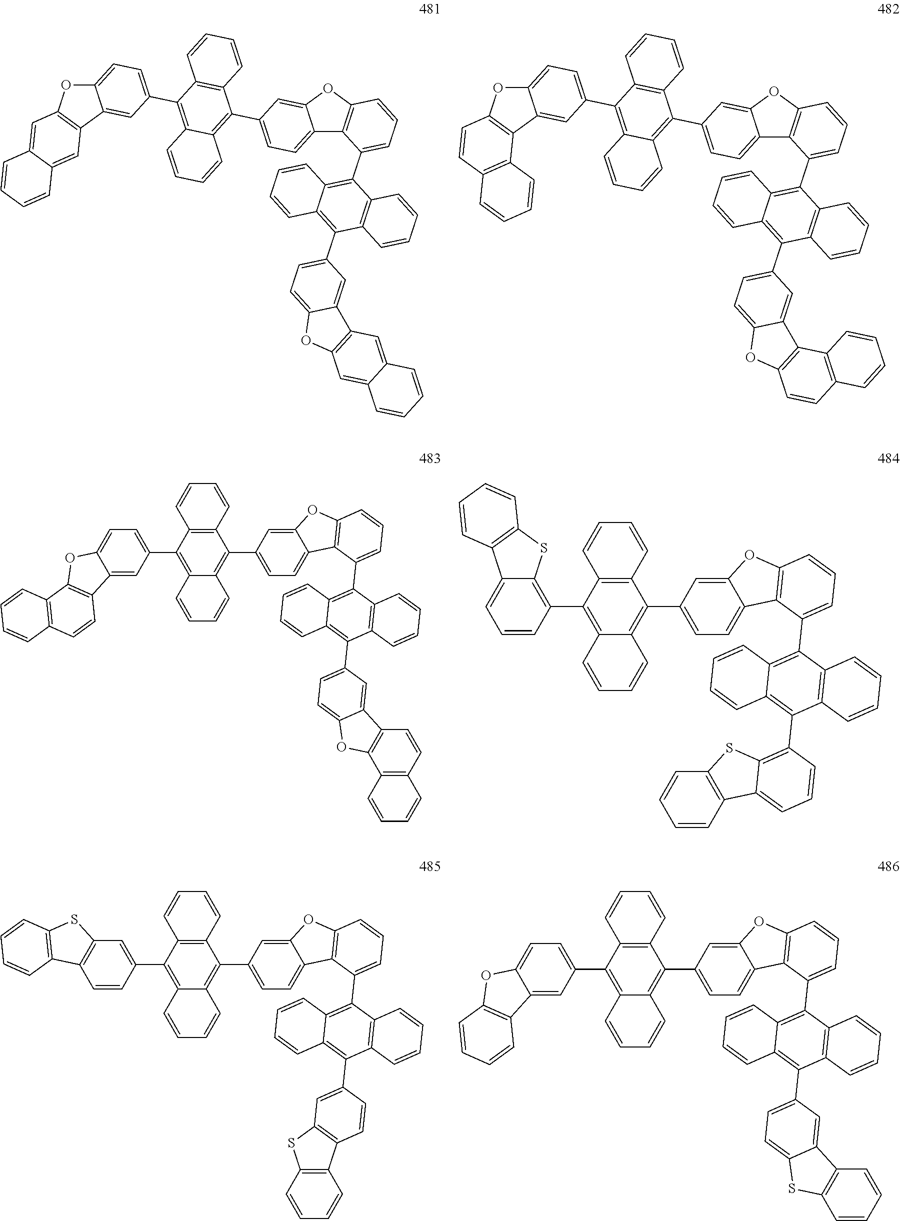

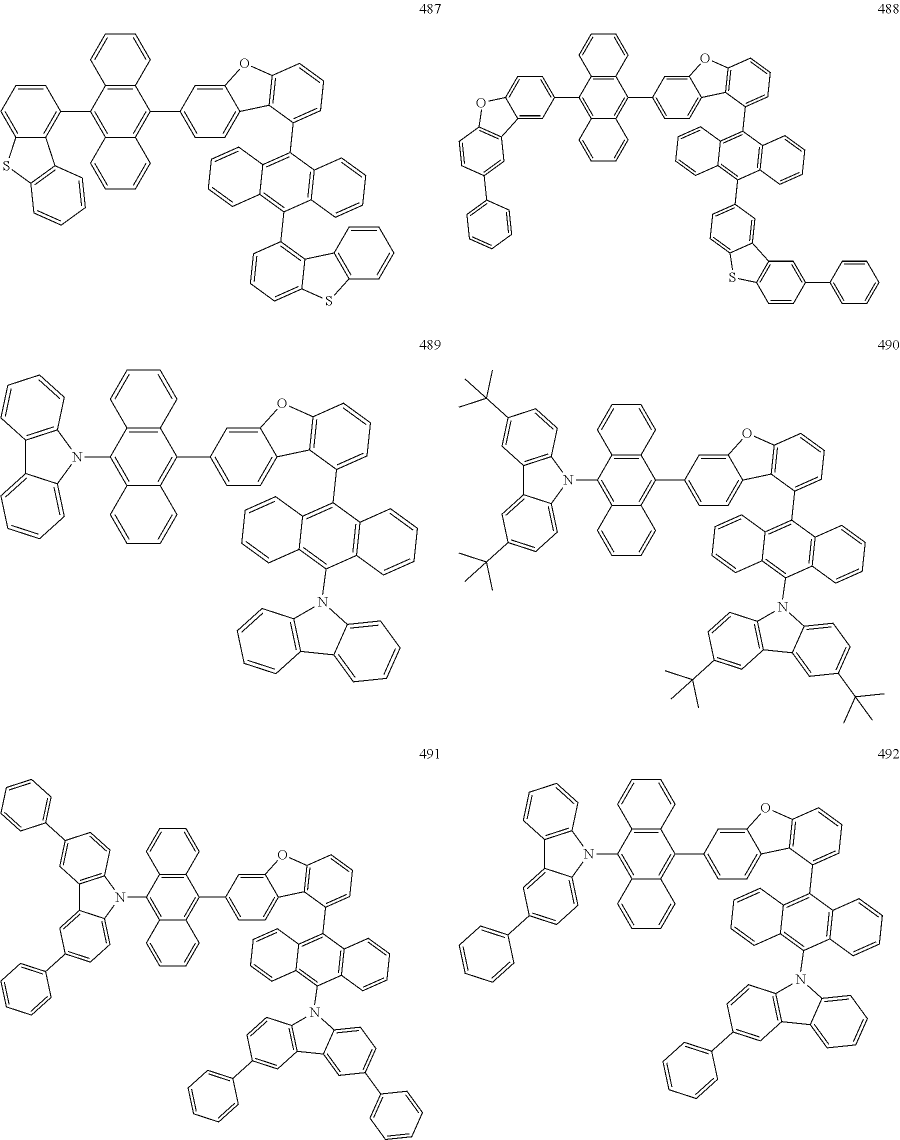

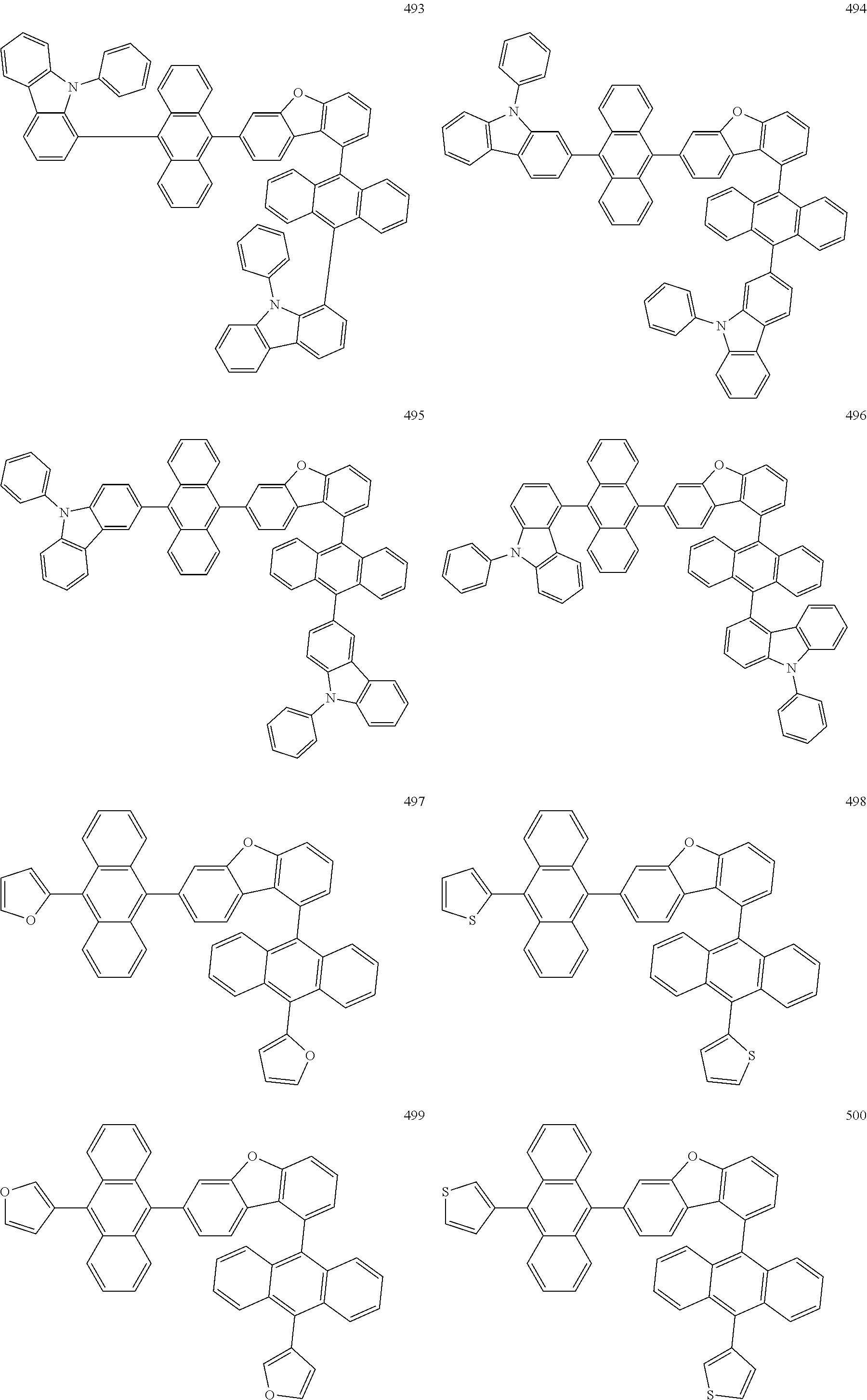

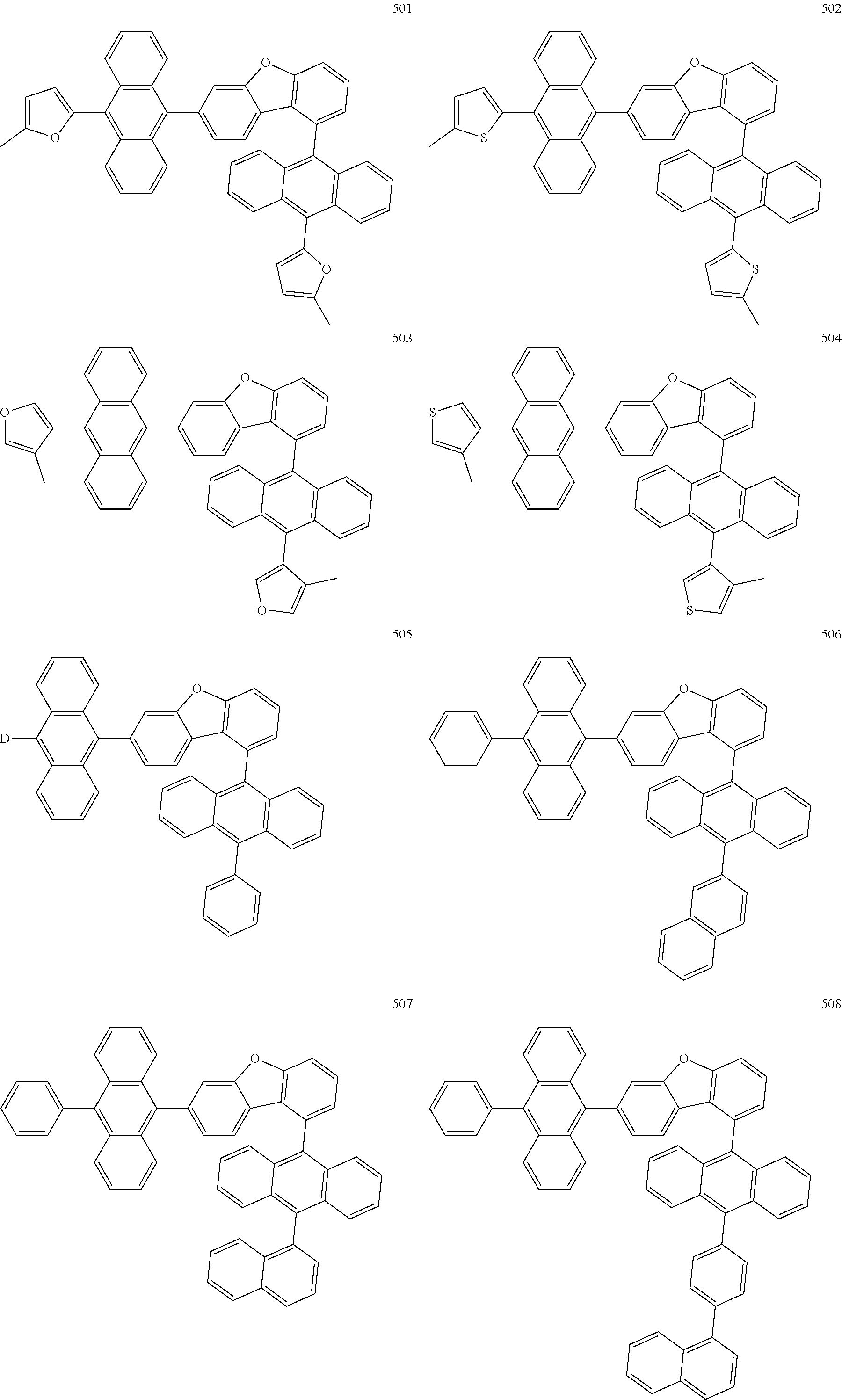

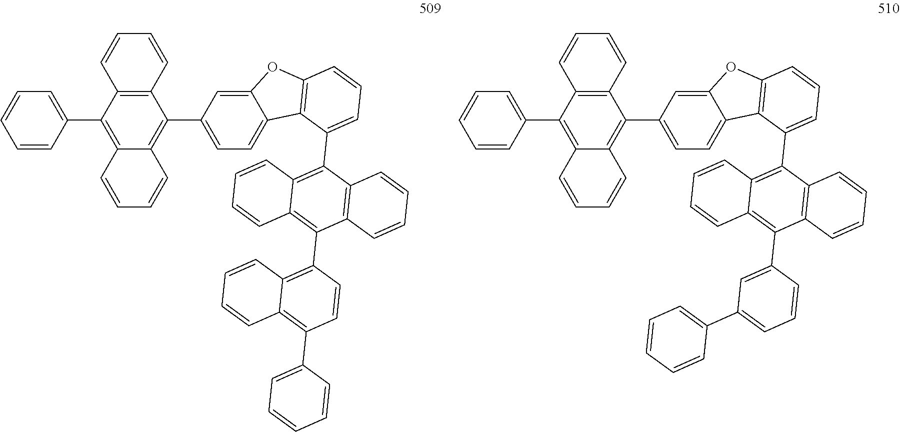 [SMILES: c1ccc(-c2ccc(-c3c4ccccc4c(-c4cccc5oc6cc(-c7c8ccccc8c(-c8ccccc8)c8ccccc78)ccc6c45)c4ccccc34)c3ccccc23)cc1 ; c1ccc(-c2cccc(-c3c4ccccc4c(-c4cccc5oc6cc(-c7c8ccccc8c(-c8ccccc8)c8ccccc78)ccc6c45)c4ccccc34)c2)cc1]

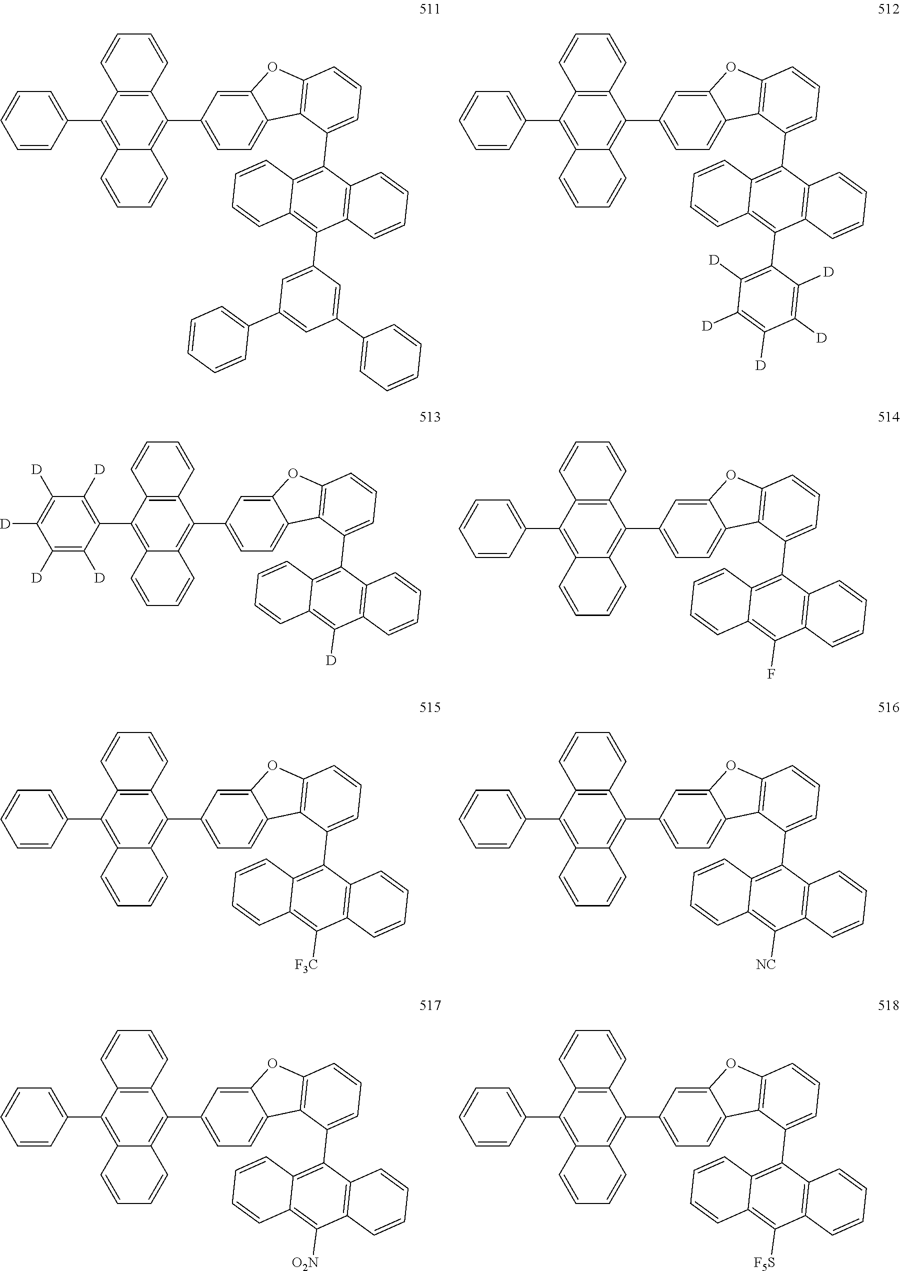

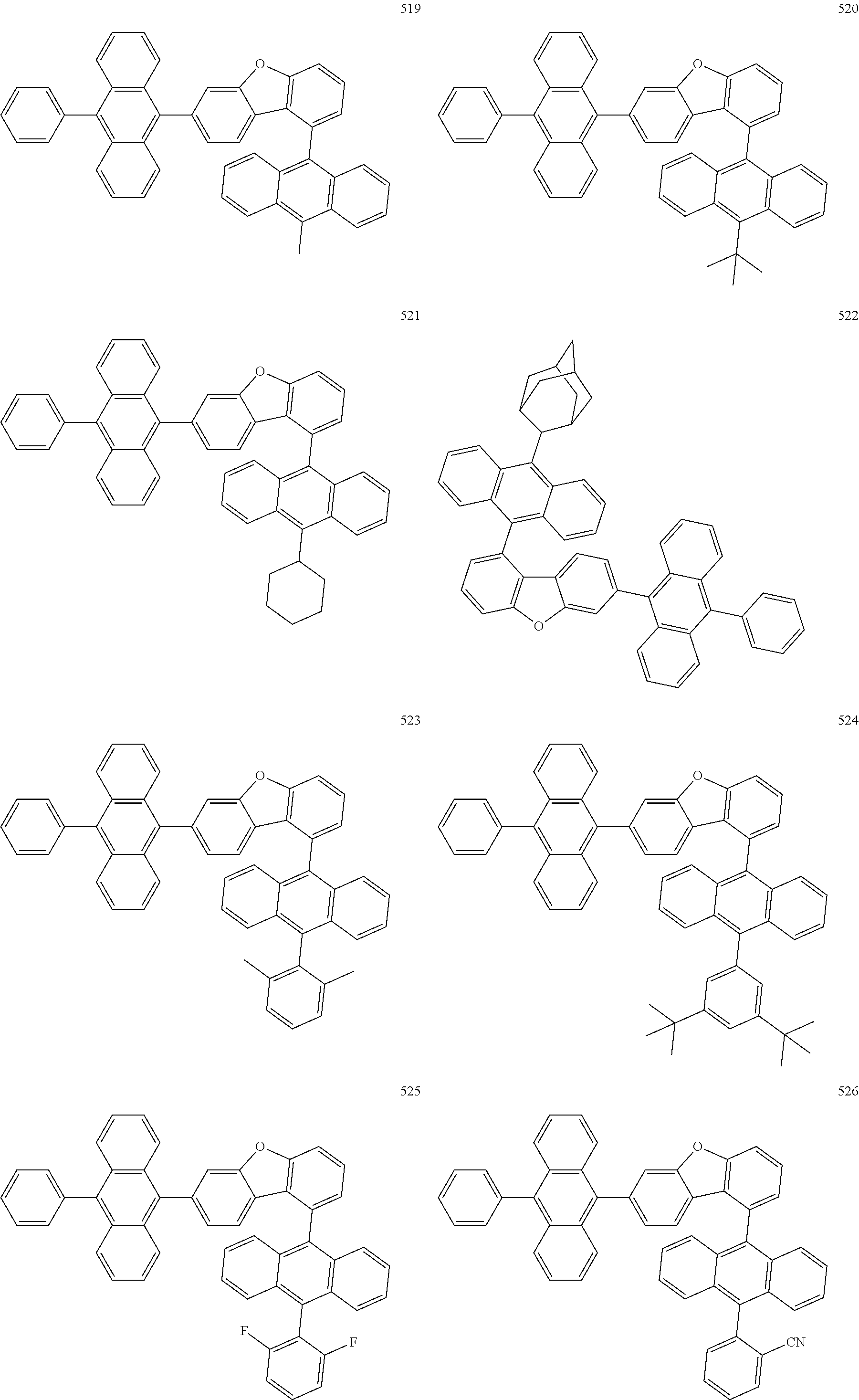

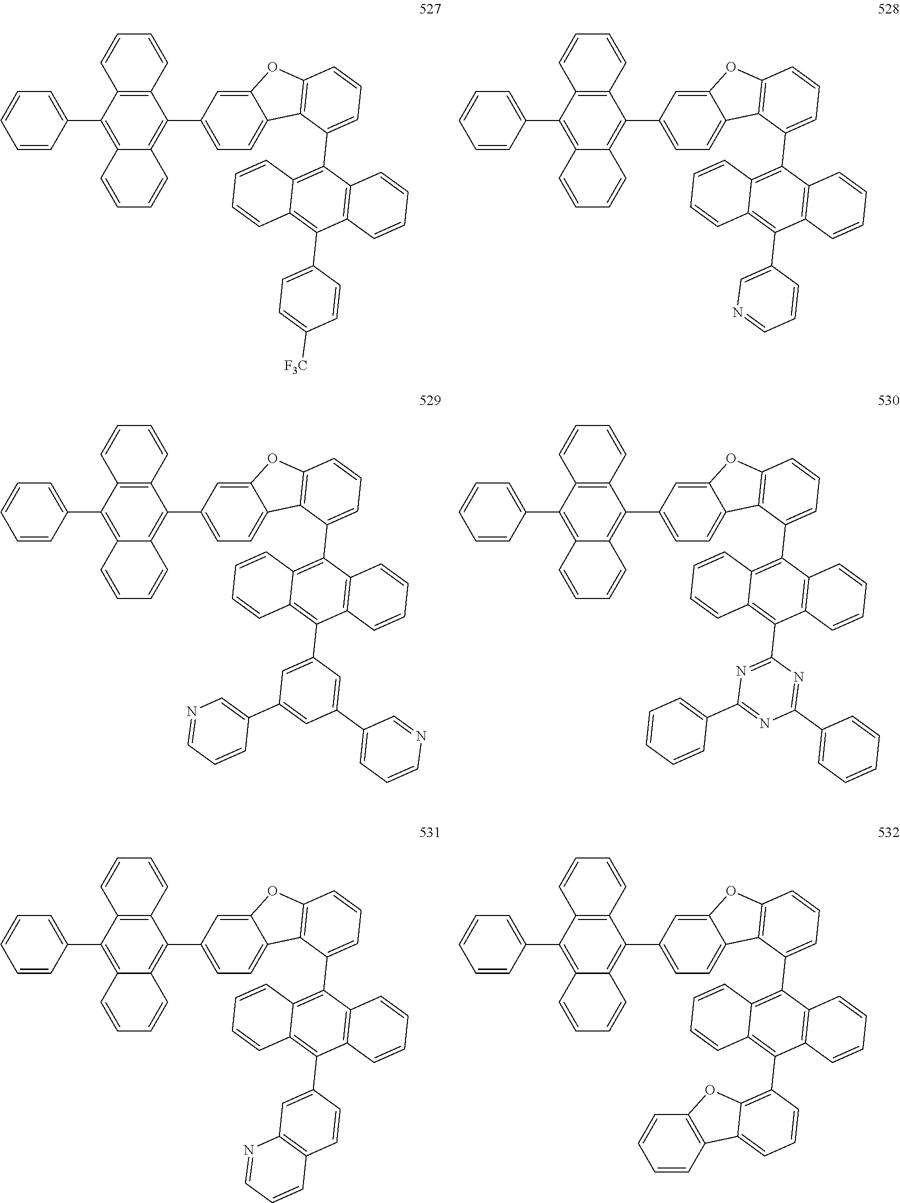

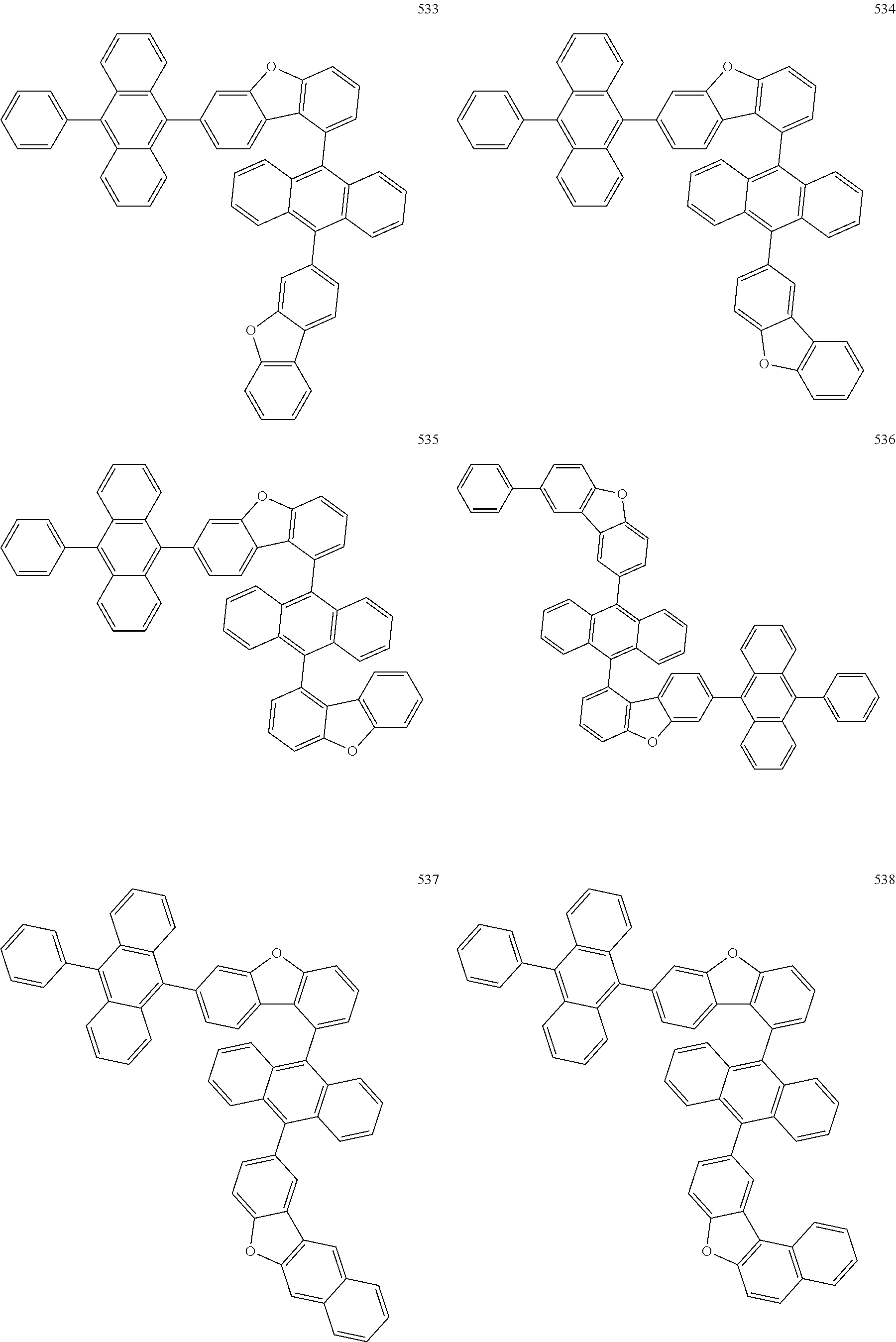

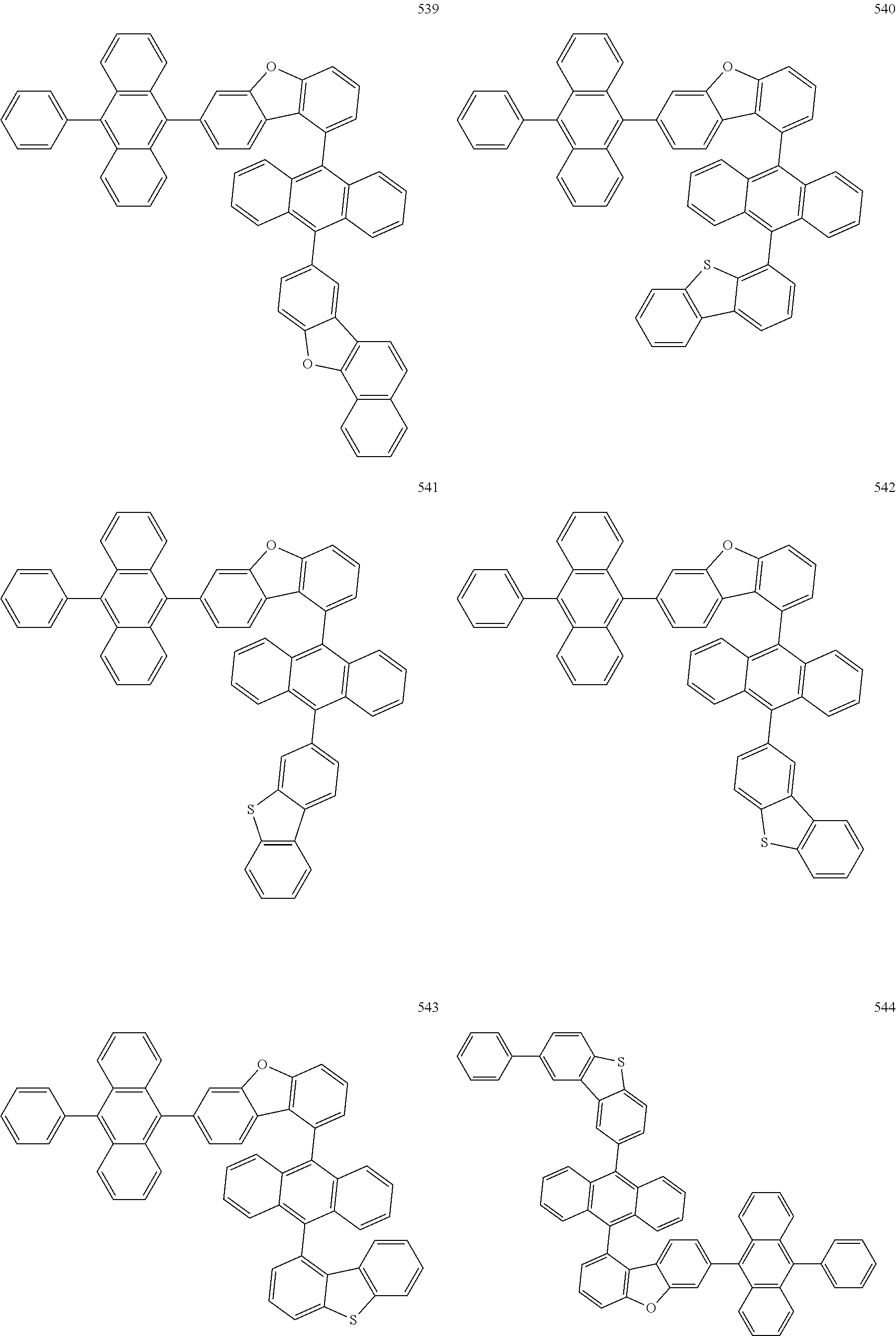

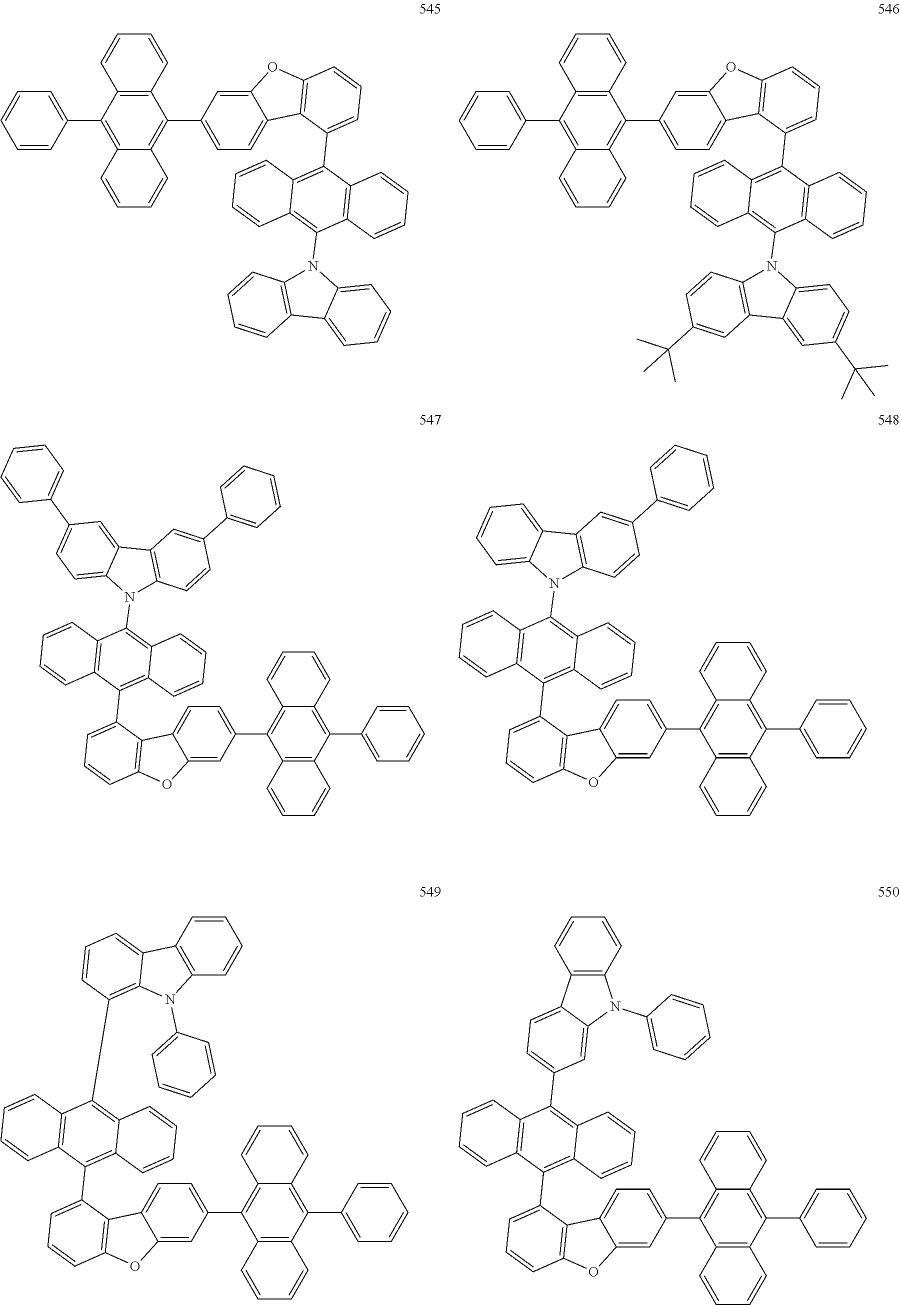

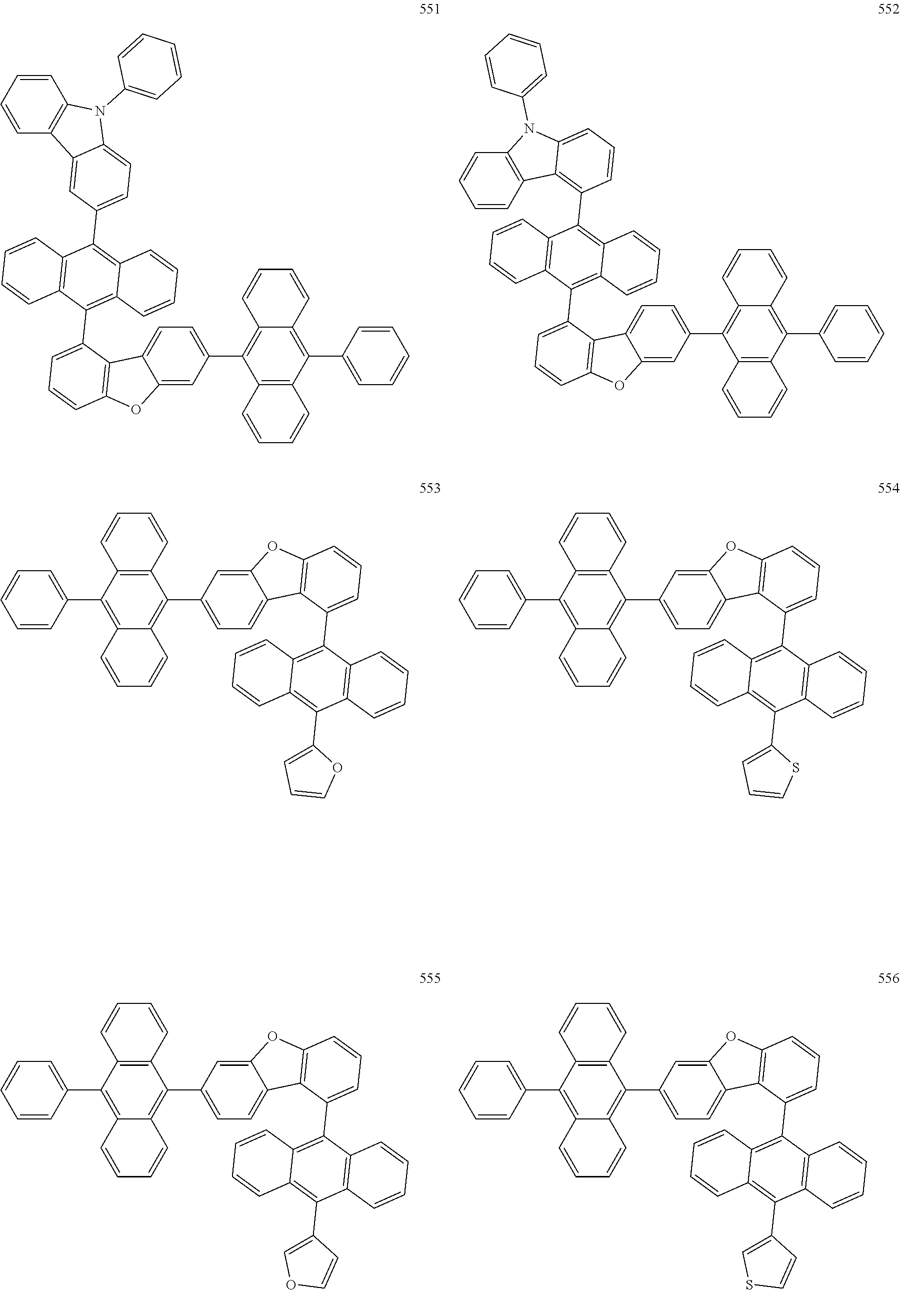

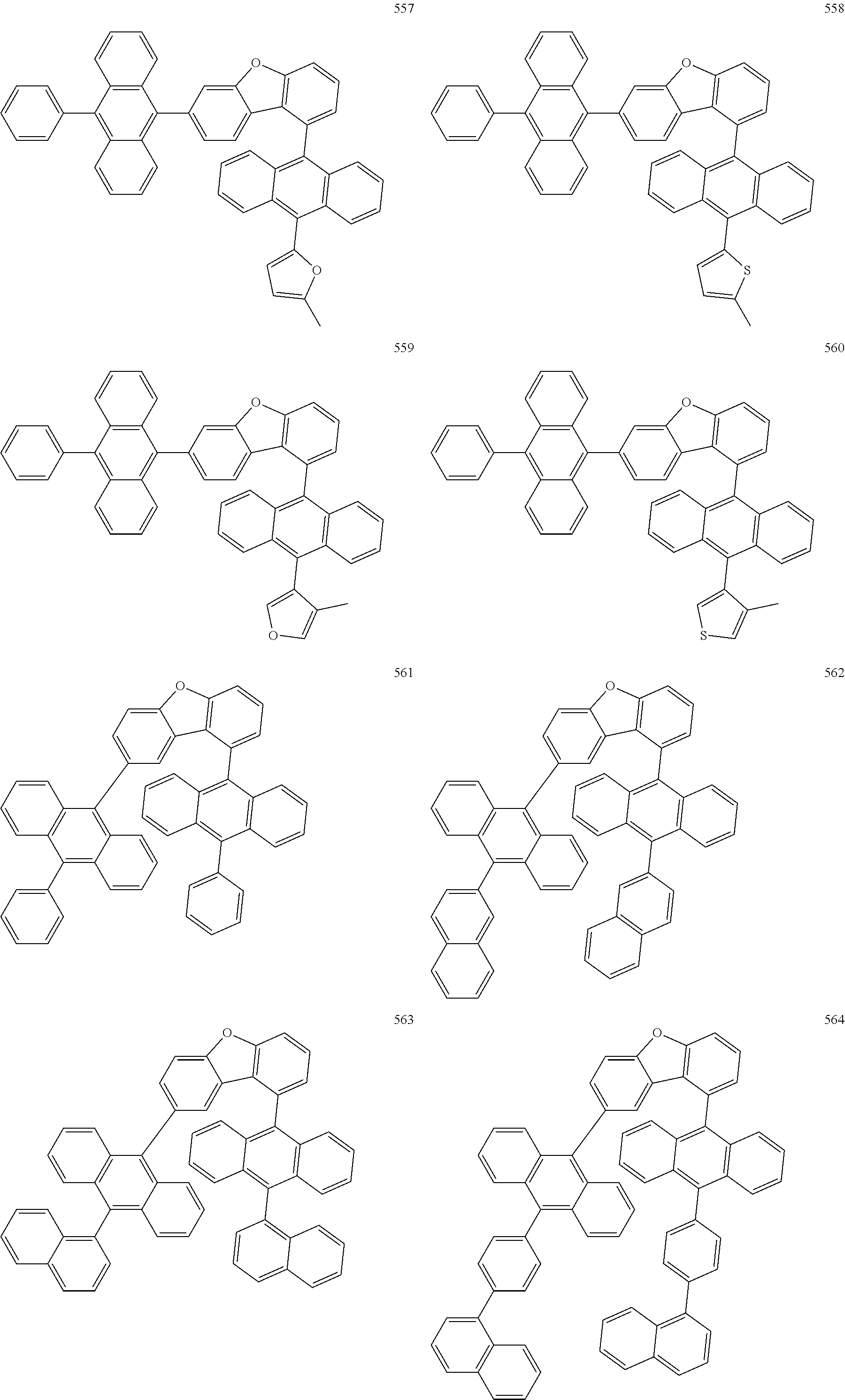

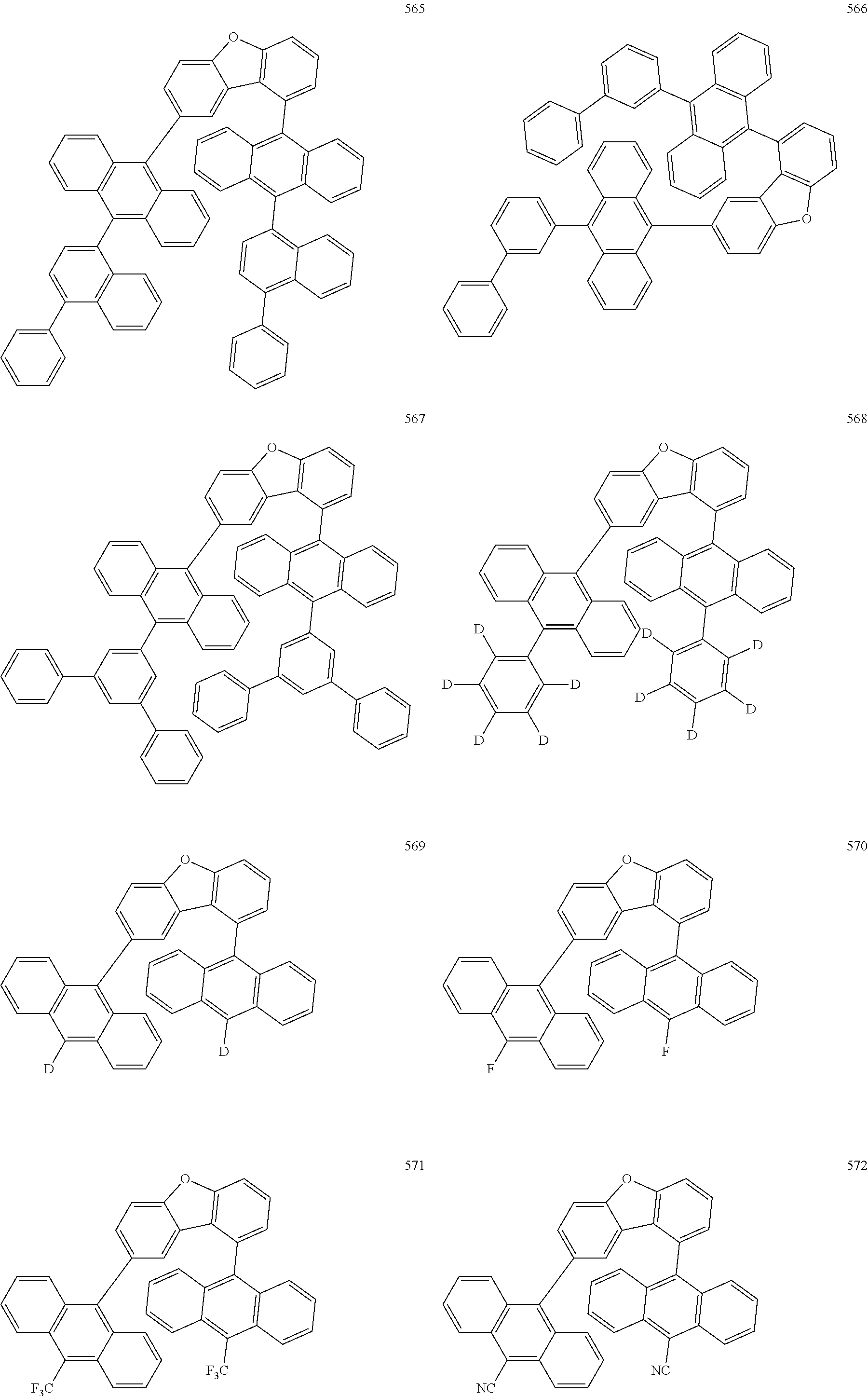

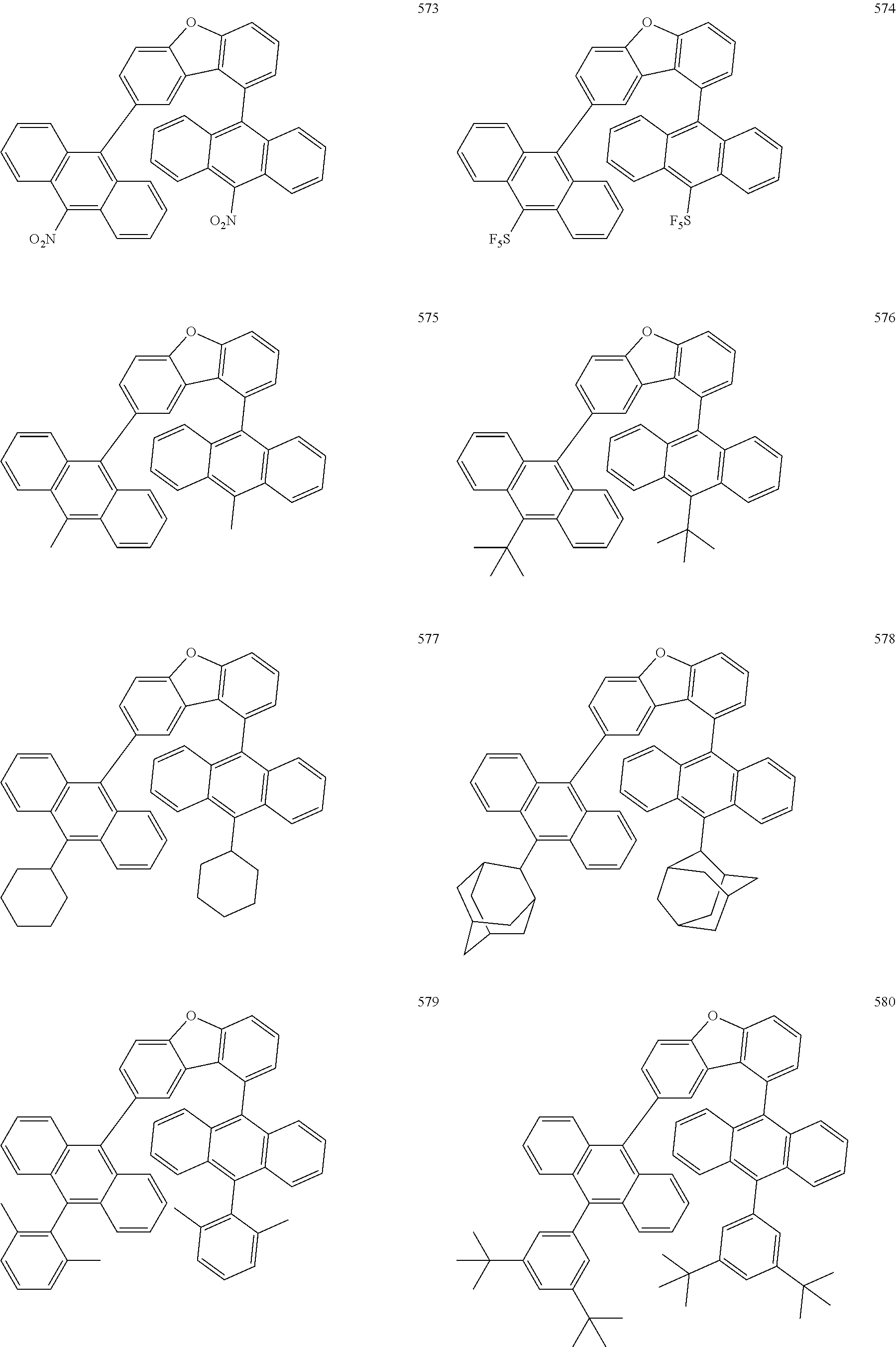

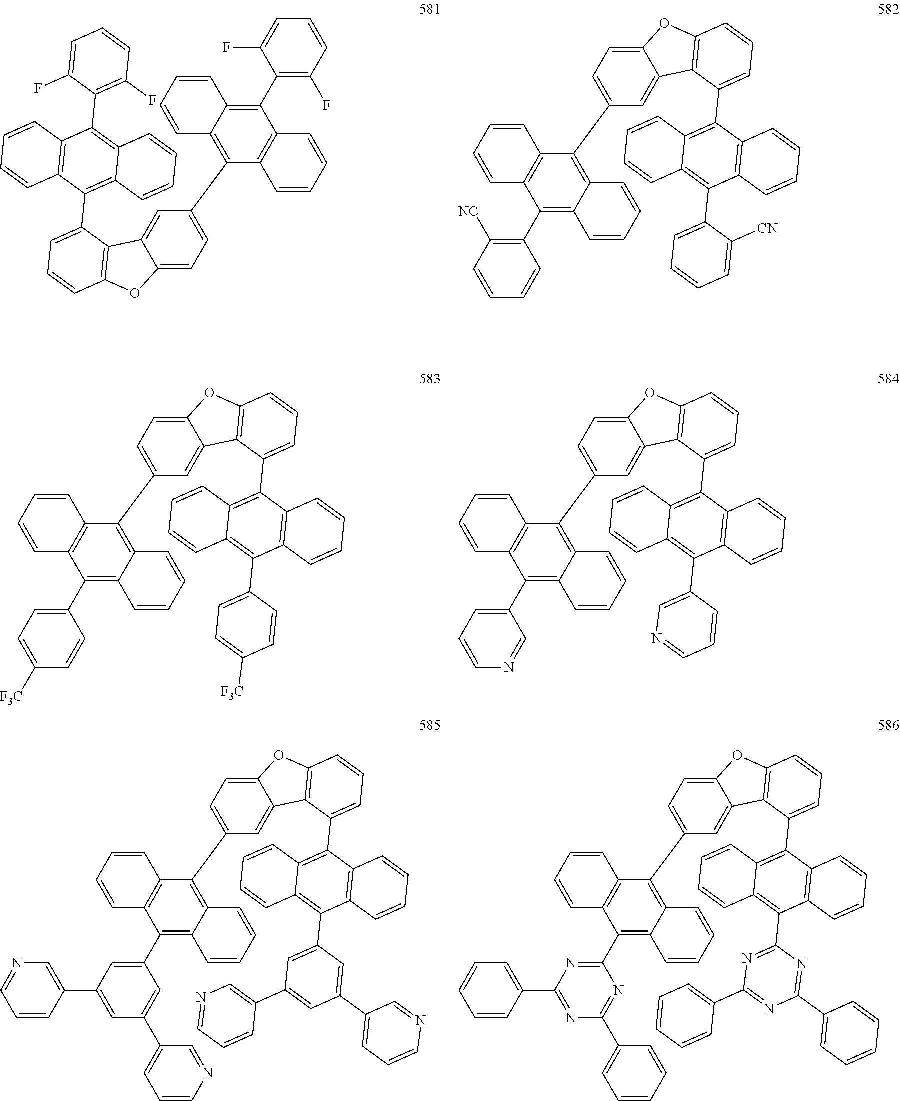

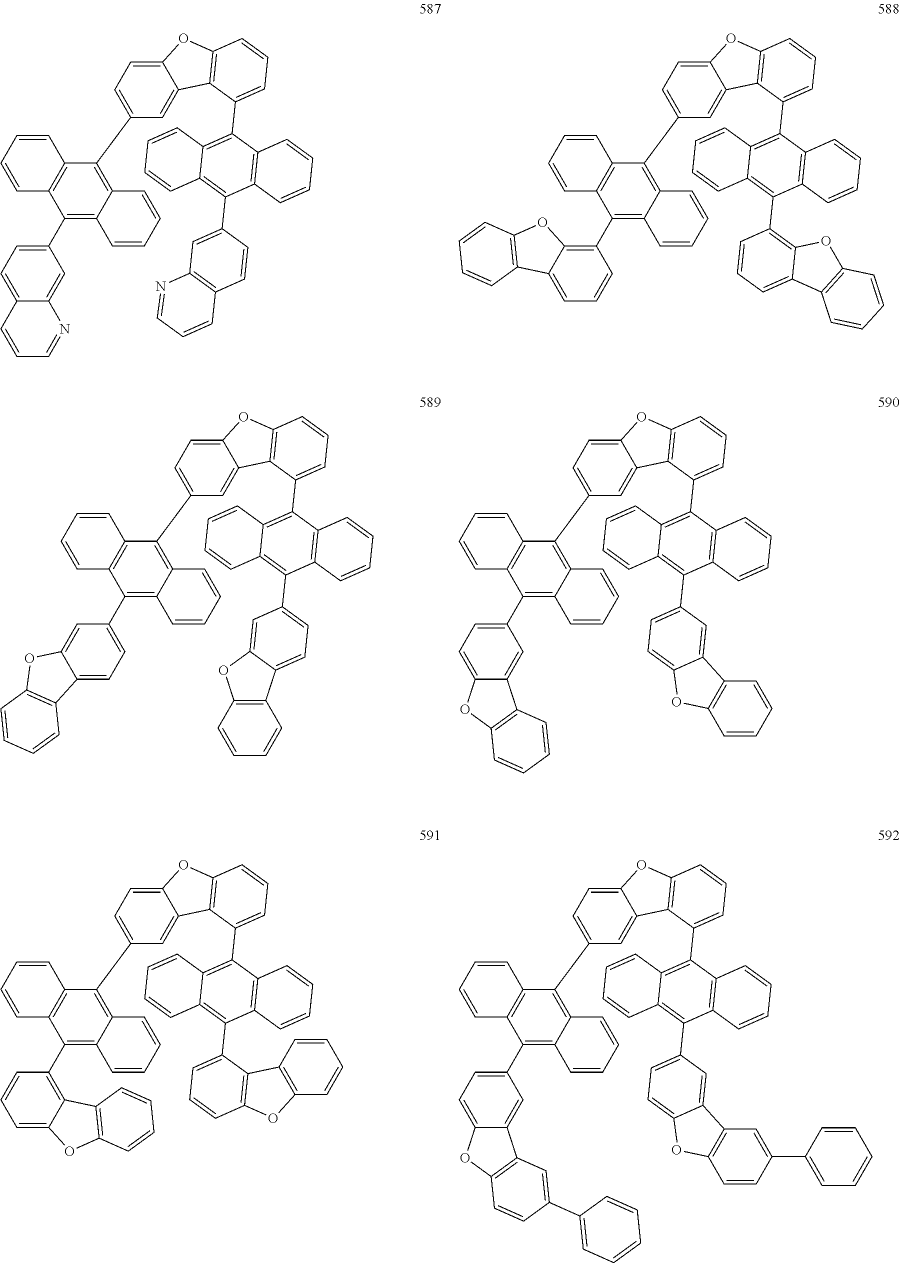

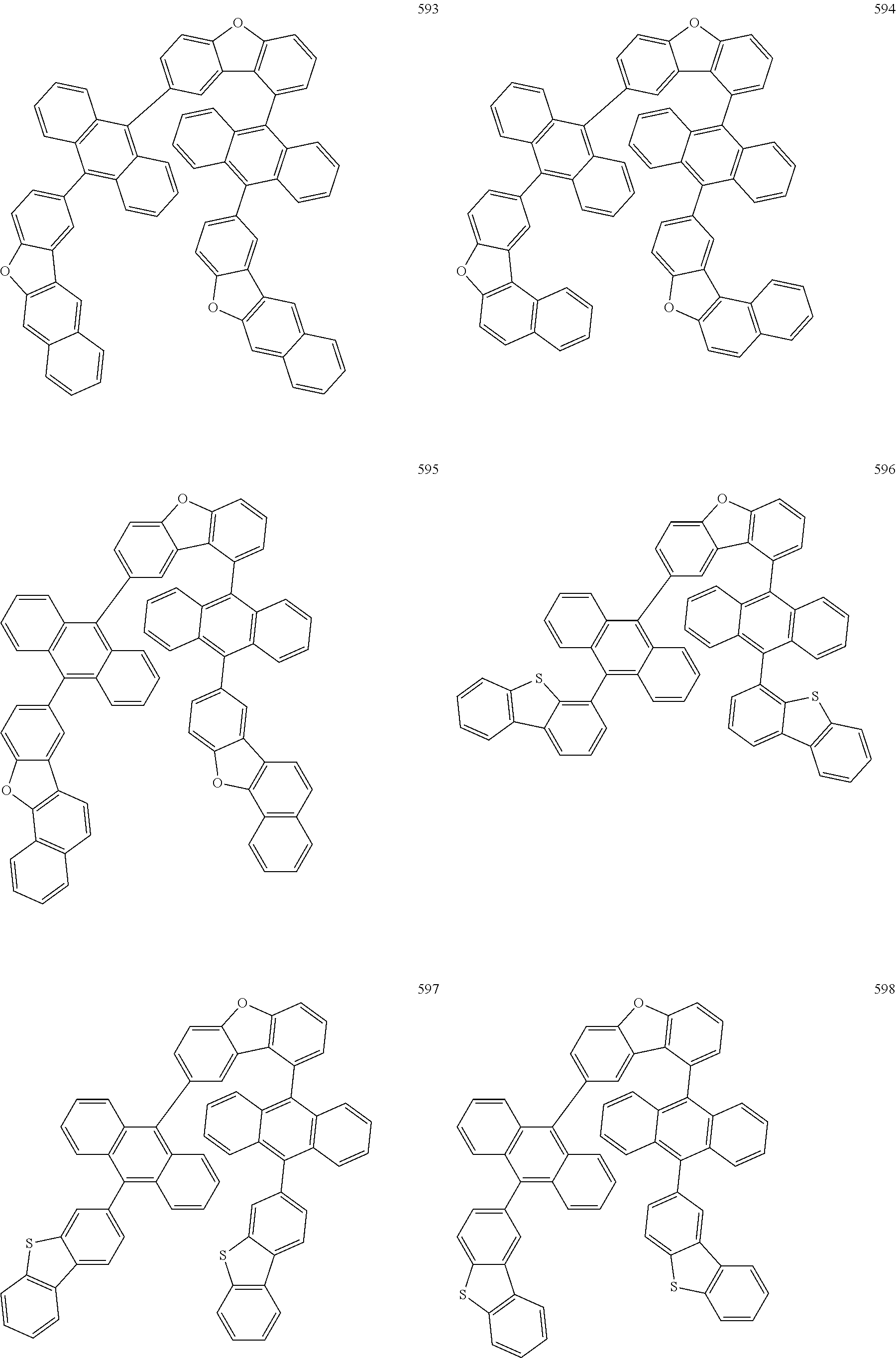

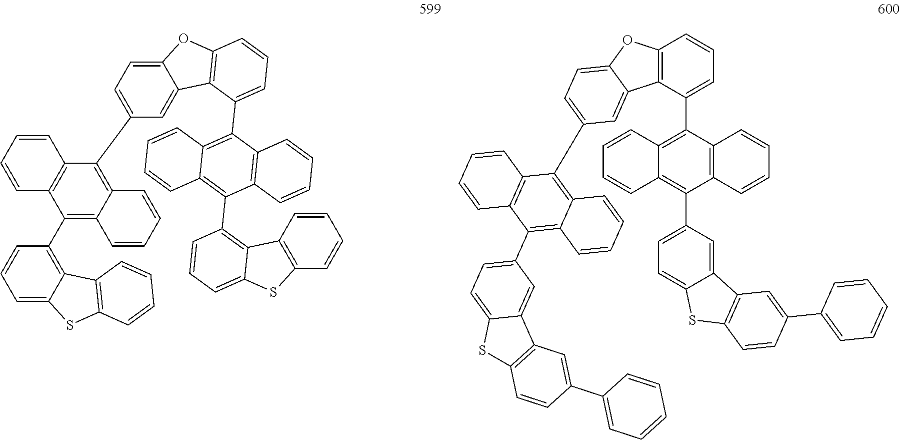 [SMILES: c1cc(-c2c3ccccc3c(-c3cccc4sc5ccccc5c34)c3ccccc23)c2c(c1)oc1ccc(-c3c4ccccc4c(-c4cccc5sc6ccccc6c45)c4ccccc34)cc12 ; c1ccc(-c2ccc3sc4ccc(-c5c6ccccc6c(-c6ccc7oc8cccc(-c9c%10ccccc%10c(-c%10ccc%11sc%12ccc(-c%13ccccc%13)cc%12c%11c%10)c%10ccccc9%10)c8c7c6)c6ccccc56)cc4c3c2)cc1]

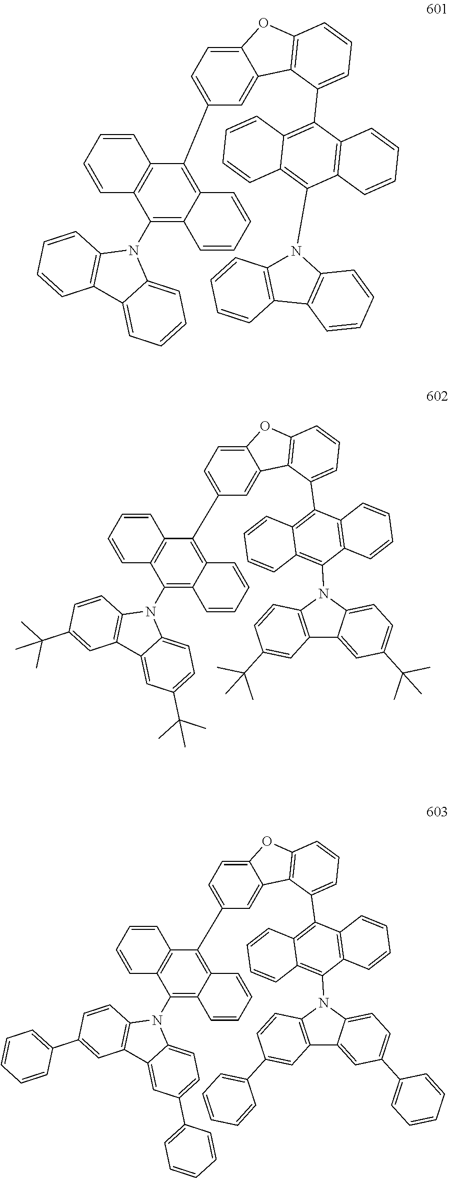 [SMILES: c1cc(-c2c3ccccc3c(-n3c4ccccc4c4ccccc43)c3ccccc23)c2c(c1)oc1ccc(-c3c4ccccc4c(-n4c5ccccc5c5ccccc54)c4ccccc34)cc12 ; CC(C)(C)c1ccc2c(c1)c1cc(C(C)(C)C)ccc1n2-c1c2ccccc2c(-c2ccc3oc4cccc(-c5c6ccccc6c(-n6c7ccc(C(C)(C)C)cc7c7cc(C(C)(C)C)ccc76)c6ccccc56)c4c3c2)c2ccccc12 ; c1ccc(-c2ccc3c(c2)c2cc(-c4ccccc4)ccc2n3-c2c3ccccc3c(-c3ccc4oc5cccc(-c6c7ccccc7c(-n7c8ccc(-c9ccccc9)cc8c8cc(-c9ccccc9)ccc87)c7ccccc67)c5c4c3)c3ccccc23)cc1]

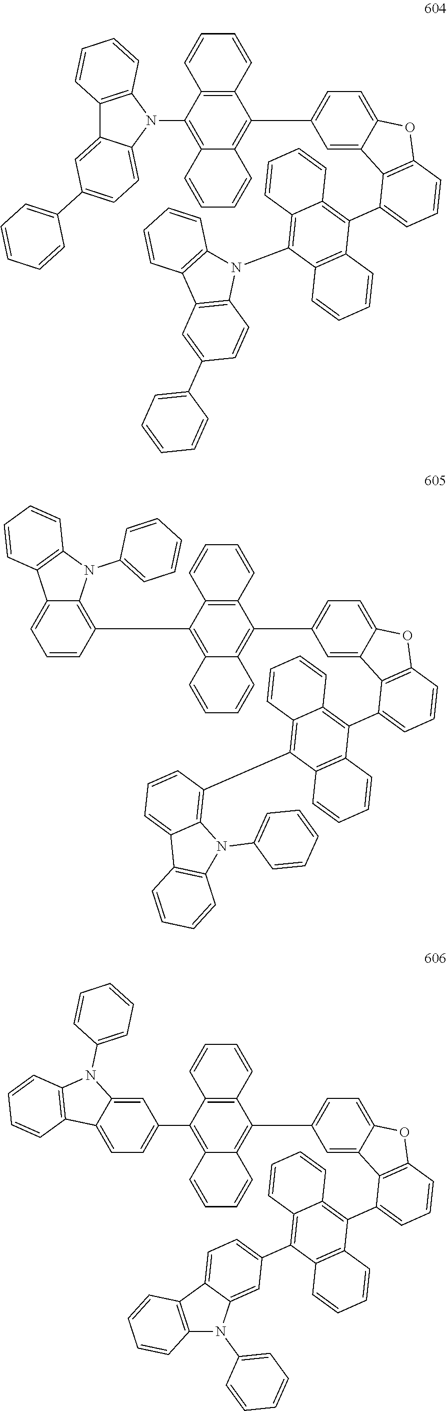 [SMILES: c1ccc(-c2ccc3c(c2)c2ccccc2n3-c2c3ccccc3c(-c3ccc4oc5cccc(-c6c7ccccc7c(-n7c8ccccc8c8cc(-c9ccccc9)ccc87)c7ccccc67)c5c4c3)c3ccccc23)cc1 ; c1ccc(-n2c3ccccc3c3cccc(-c4c5ccccc5c(-c5ccc6oc7cccc(-c8c9ccccc9c(-c9cccc%10c%11ccccc%11n(-c%11ccccc%11)c9%10)c9ccccc89)c7c6c5)c5ccccc45)c32)cc1 ; c1ccc(-n2c3ccccc3c3ccc(-c4c5ccccc5c(-c5ccc6oc7cccc(-c8c9ccccc9c(-c9ccc%10c%11ccccc%11n(-c%11ccccc%11)c%10c9)c9ccccc89)c7c6c5)c5ccccc45)cc32)cc1]

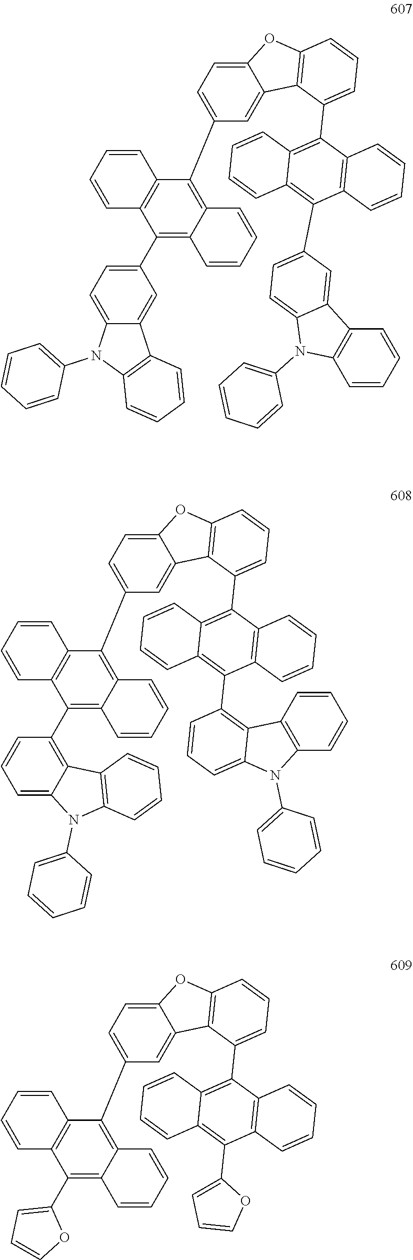 [SMILES: c1ccc(-n2c3ccccc3c3cc(-c4c5ccccc5c(-c5ccc6oc7cccc(-c8c9ccccc9c(-c9ccc%10c(c9)c9ccccc9n%10-c9ccccc9)c9ccccc89)c7c6c5)c5ccccc45)ccc32)cc1 ; c1ccc(-n2c3ccccc3c3c(-c4c5ccccc5c(-c5ccc6oc7cccc(-c8c9ccccc9c(-c9cccc%10c9c9ccccc9n%10-c9ccccc9)c9ccccc89)c7c6c5)c5ccccc45)cccc32)cc1 ; c1coc(-c2c3ccccc3c(-c3ccc4oc5cccc(-c6c7ccccc7c(-c7ccco7)c7ccccc67)c5c4c3)c3ccccc23)c1]

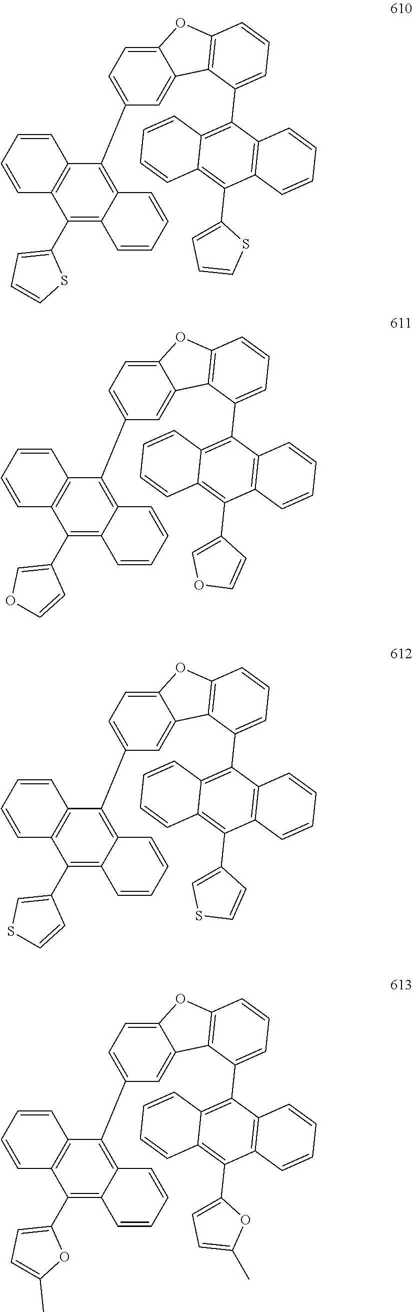 [SMILES: c1csc(-c2c3ccccc3c(-c3ccc4oc5cccc(-c6c7ccccc7c(-c7cccs7)c7ccccc67)c5c4c3)c3ccccc23)c1 ; c1cc(-c2c3ccccc3c(-c3ccoc3)c3ccccc23)c2c(c1)oc1ccc(-c3c4ccccc4c(-c4ccoc4)c4ccccc34)cc12 ; c1cc(-c2c3ccccc3c(-c3ccsc3)c3ccccc23)c2c(c1)oc1ccc(-c3c4ccccc4c(-c4ccsc4)c4ccccc34)cc12 ; Cc1ccc(-c2c3ccccc3c(-c3ccc4oc5cccc(-c6c7ccccc7c(-c7ccc(C)o7)c7ccccc67)c5c4c3)c3ccccc23)o1]

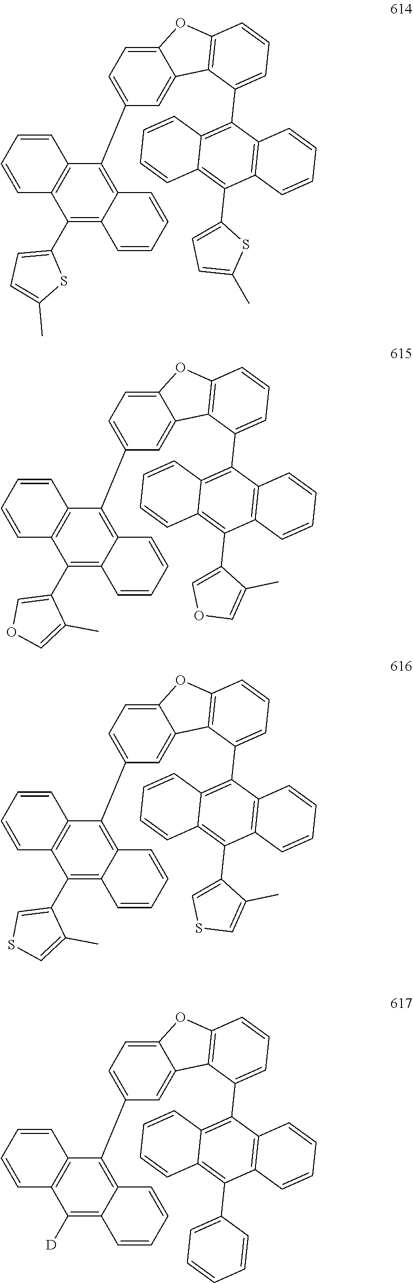 [SMILES: Cc1ccc(-c2c3ccccc3c(-c3ccc4oc5cccc(-c6c7ccccc7c(-c7ccc(C)s7)c7ccccc67)c5c4c3)c3ccccc23)s1 ; Cc1cocc1-c1c2ccccc2c(-c2ccc3oc4cccc(-c5c6ccccc6c(-c6cocc6C)c6ccccc56)c4c3c2)c2ccccc12 ; Cc1cscc1-c1c2ccccc2c(-c2ccc3oc4cccc(-c5c6ccccc6c(-c6cscc6C)c6ccccc56)c4c3c2)c2ccccc12 ; [2H]c1c2ccccc2c(-c2ccc3oc4cccc(-c5c6ccccc6c(-c6ccccc6)c6ccccc56)c4c3c2)c2ccccc12]

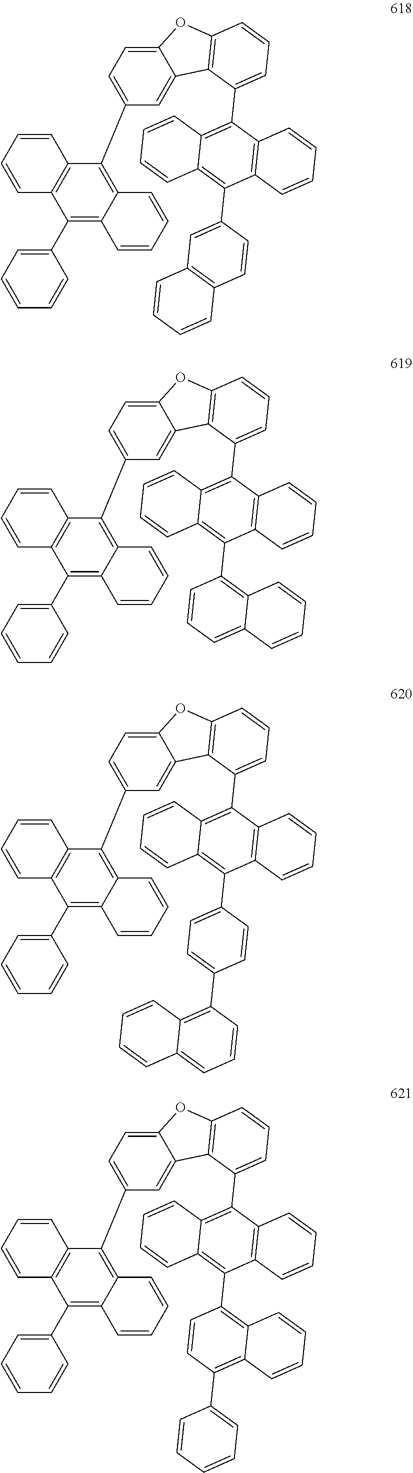

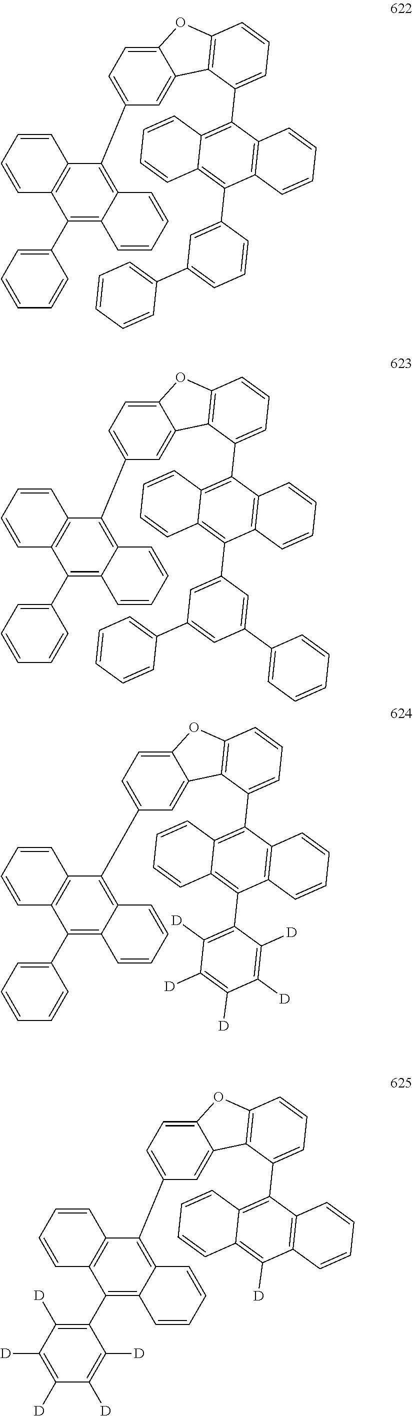 [SMILES: c1ccc(-c2cccc(-c3c4ccccc4c(-c4cccc5oc6ccc(-c7c8ccccc8c(-c8ccccc8)c8ccccc78)cc6c45)c4ccccc34)c2)cc1 ; c1ccc(-c2cc(-c3ccccc3)cc(-c3c4ccccc4c(-c4cccc5oc6ccc(-c7c8ccccc8c(-c8ccccc8)c8ccccc78)cc6c45)c4ccccc34)c2)cc1 ; [2H]c1c([2H])c([2H])c(-c2c3ccccc3c(-c3cccc4oc5ccc(-c6c7ccccc7c(-c7ccccc7)c7ccccc67)cc5c34)c3ccccc23)c([2H])c1[2H] ; [2H]c1c([2H])c([2H])c(-c2c3ccccc3c(-c3ccc4oc5cccc(-c6c7ccccc7c([2H])c7ccccc67)c5c4c3)c3ccccc23)c([2H])c1[2H]]

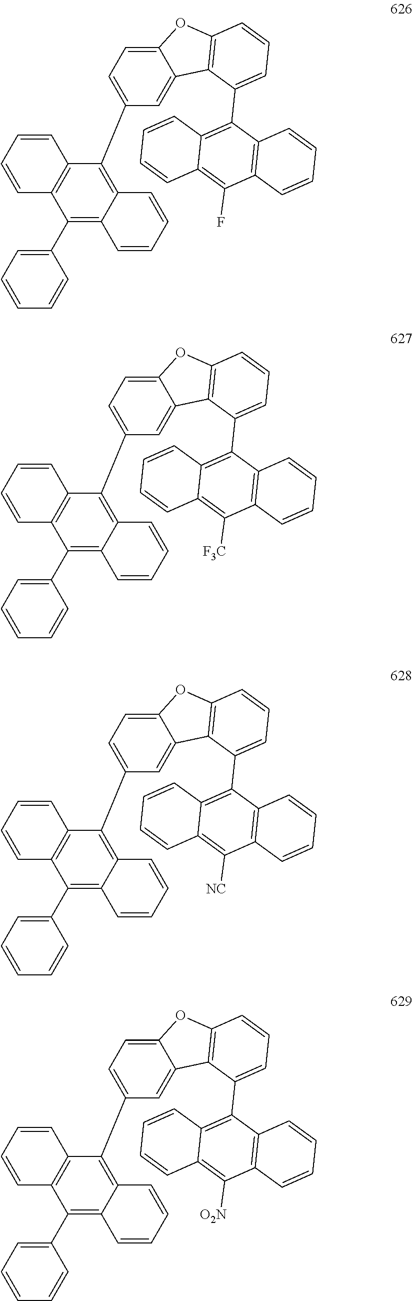 [SMILES: Fc1c2ccccc2c(-c2cccc3oc4ccc(-c5c6ccccc6c(-c6ccccc6)c6ccccc56)cc4c23)c2ccccc12 ; FC(F)(F)c1c2ccccc2c(-c2cccc3oc4ccc(-c5c6ccccc6c(-c6ccccc6)c6ccccc56)cc4c23)c2ccccc12 ; N#Cc1c2ccccc2c(-c2cccc3oc4ccc(-c5c6ccccc6c(-c6ccccc6)c6ccccc56)cc4c23)c2ccccc12 ; O=[N+]([O-])c1c2ccccc2c(-c2cccc3oc4ccc(-c5c6ccccc6c(-c6ccccc6)c6ccccc56)cc4c23)c2ccccc12]

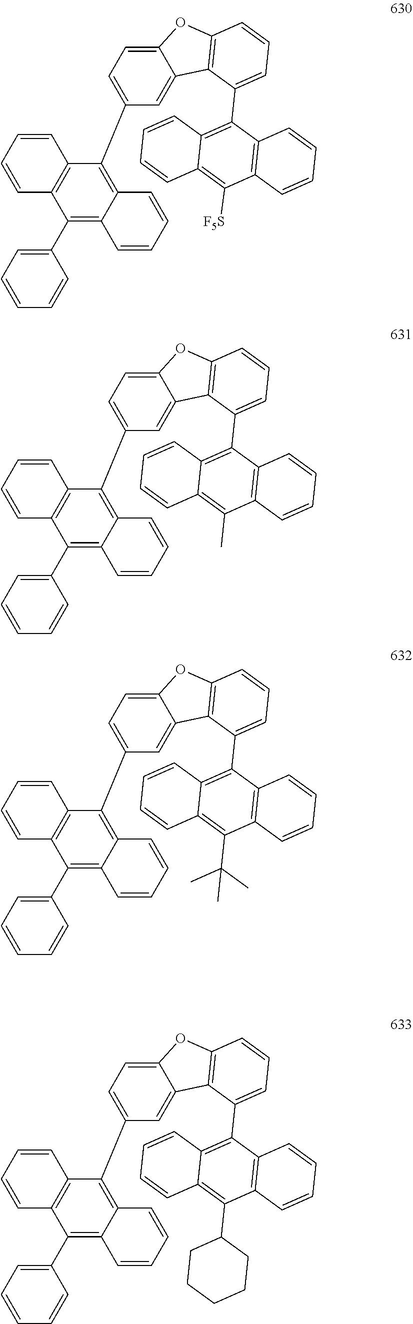

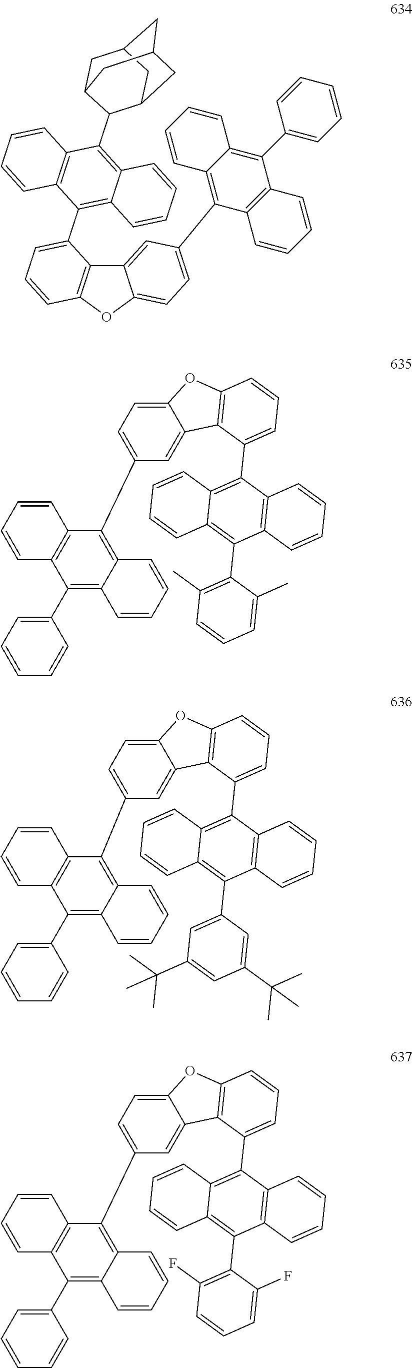 [SMILES: c1ccc(-c2c3ccccc3c(-c3ccc4oc5cccc(-c6c7ccccc7c(C7C8CC9CC(C8)CC7C9)c7ccccc67)c5c4c3)c3ccccc23)cc1 ; Cc1cccc(C)c1-c1c2ccccc2c(-c2cccc3oc4ccc(-c5c6ccccc6c(-c6ccccc6)c6ccccc56)cc4c23)c2ccccc12 ; CC(C)(C)c1cc(-c2c3ccccc3c(-c3cccc4oc5ccc(-c6c7ccccc7c(-c7ccccc7)c7ccccc67)cc5c34)c3ccccc23)cc(C(C)(C)C)c1 ; Fc1cccc(F)c1-c1c2ccccc2c(-c2cccc3oc4ccc(-c5c6ccccc6c(-c6ccccc6)c6ccccc56)cc4c23)c2ccccc12]

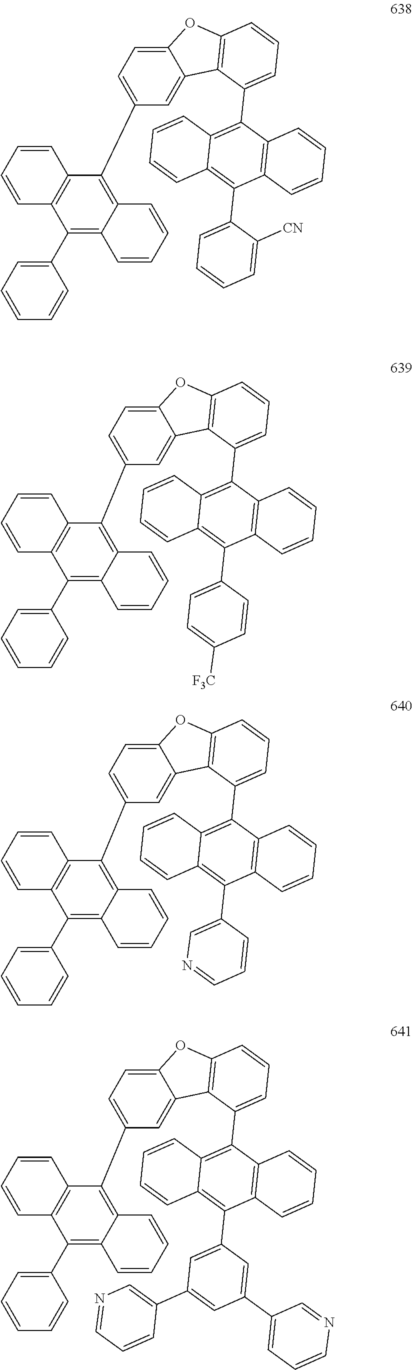 [SMILES: N#Cc1ccccc1-c1c2ccccc2c(-c2cccc3oc4ccc(-c5c6ccccc6c(-c6ccccc6)c6ccccc56)cc4c23)c2ccccc12 ; FC(F)(F)c1ccc(-c2c3ccccc3c(-c3cccc4oc5ccc(-c6c7ccccc7c(-c7ccccc7)c7ccccc67)cc5c34)c3ccccc23)cc1 ; c1ccc(-c2c3ccccc3c(-c3ccc4oc5cccc(-c6c7ccccc7c(-c7cccnc7)c7ccccc67)c5c4c3)c3ccccc23)cc1 ; c1ccc(-c2c3ccccc3c(-c3ccc4oc5cccc(-c6c7ccccc7c(-c7cc(-c8cccnc8)cc(-c8cccnc8)c7)c7ccccc67)c5c4c3)c3ccccc23)cc1]

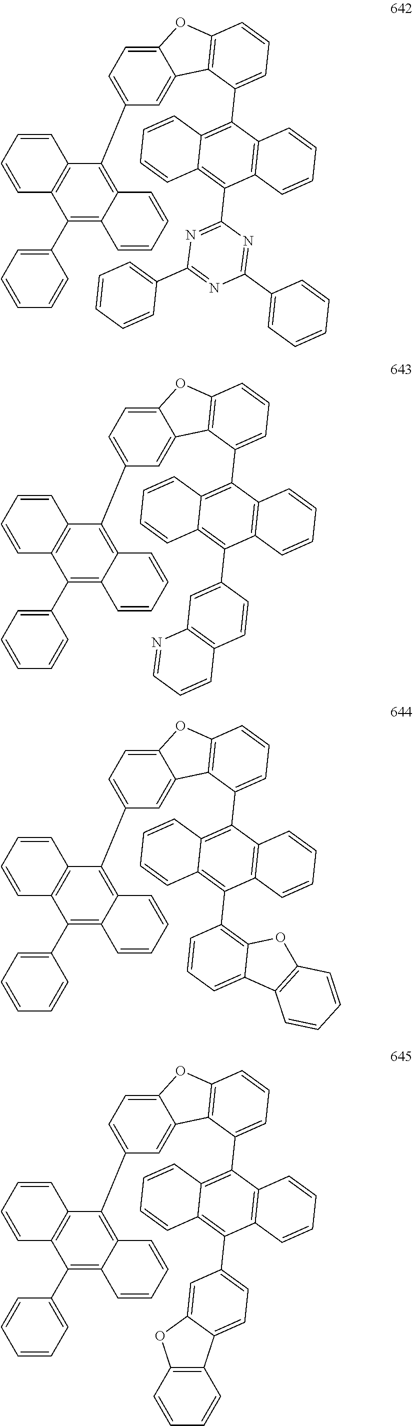

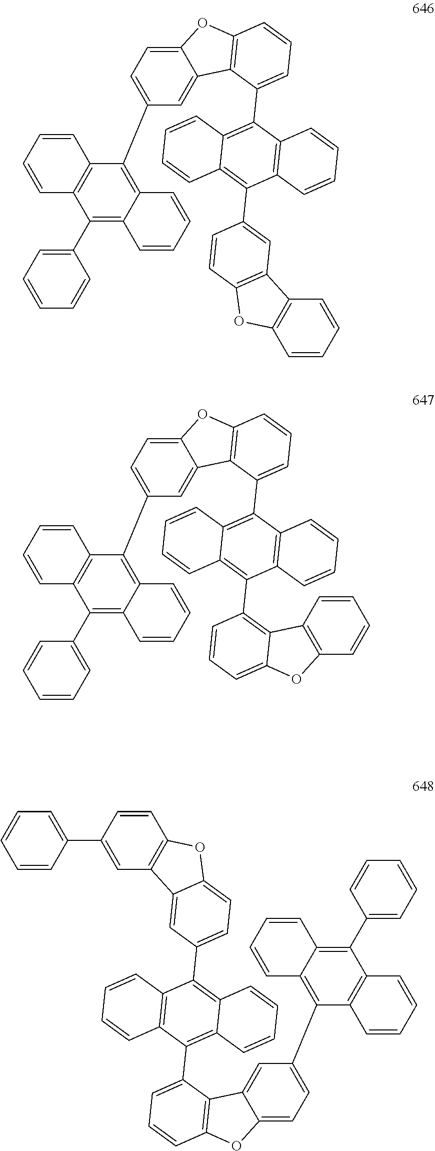 [SMILES: c1ccc(-c2c3ccccc3c(-c3ccc4oc5cccc(-c6c7ccccc7c(-c7ccc8oc9ccccc9c8c7)c7ccccc67)c5c4c3)c3ccccc23)cc1 ; c1ccc(-c2c3ccccc3c(-c3ccc4oc5cccc(-c6c7ccccc7c(-c7cccc8oc9ccccc9c78)c7ccccc67)c5c4c3)c3ccccc23)cc1 ; c1ccc(-c2ccc3oc4ccc(-c5c6ccccc6c(-c6cccc7oc8ccc(-c9c%10ccccc%10c(-c%10ccccc%10)c%10ccccc9%10)cc8c67)c6ccccc56)cc4c3c2)cc1]

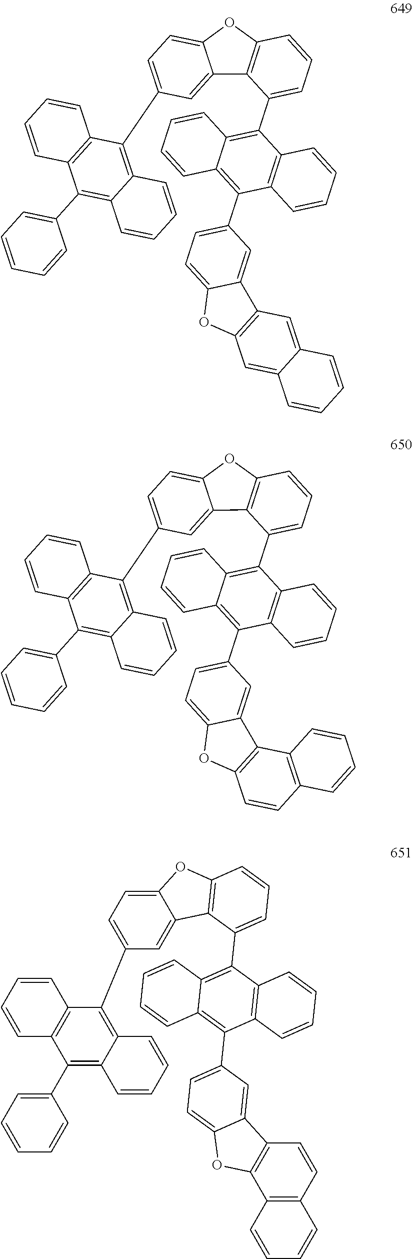 [SMILES: c1ccc(-c2c3ccccc3c(-c3ccc4oc5cccc(-c6c7ccccc7c(-c7ccc8oc9cc%10ccccc%10cc9c8c7)c7ccccc67)c5c4c3)c3ccccc23)cc1 ; c1ccc(-c2c3ccccc3c(-c3ccc4oc5cccc(-c6c7ccccc7c(-c7ccc8oc9ccc%10ccccc%10c9c8c7)c7ccccc67)c5c4c3)c3ccccc23)cc1 ; c1ccc(-c2c3ccccc3c(-c3ccc4oc5cccc(-c6c7ccccc7c(-c7ccc8oc9c%10ccccc%10ccc9c8c7)c7ccccc67)c5c4c3)c3ccccc23)cc1]

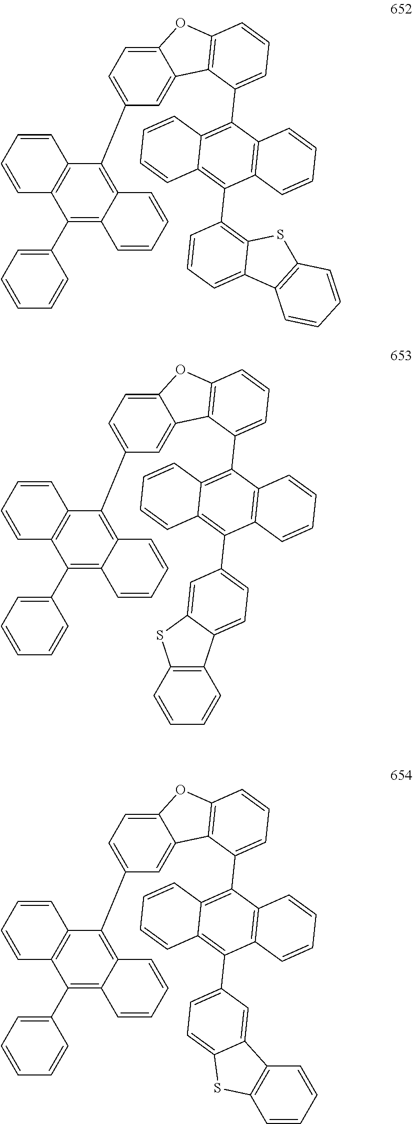 [SMILES: c1ccc(-c2c3ccccc3c(-c3ccc4oc5cccc(-c6c7ccccc7c(-c7cccc8c7sc7ccccc78)c7ccccc67)c5c4c3)c3ccccc23)cc1 ; c1ccc(-c2c3ccccc3c(-c3ccc4oc5cccc(-c6c7ccccc7c(-c7ccc8c(c7)sc7ccccc78)c7ccccc67)c5c4c3)c3ccccc23)cc1 ; c1ccc(-c2c3ccccc3c(-c3ccc4oc5cccc(-c6c7ccccc7c(-c7ccc8sc9ccccc9c8c7)c7ccccc67)c5c4c3)c3ccccc23)cc1]

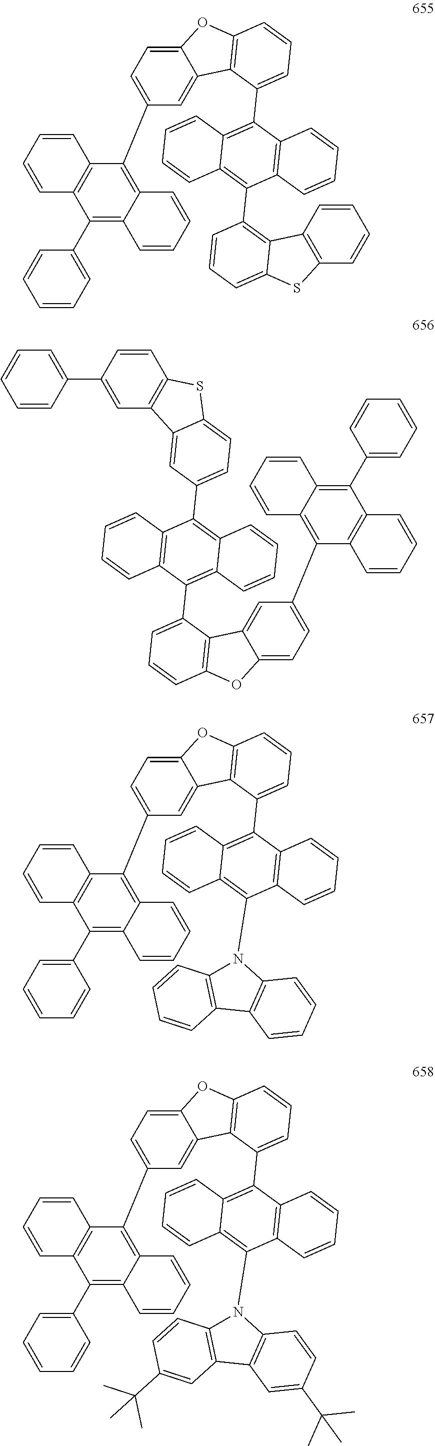

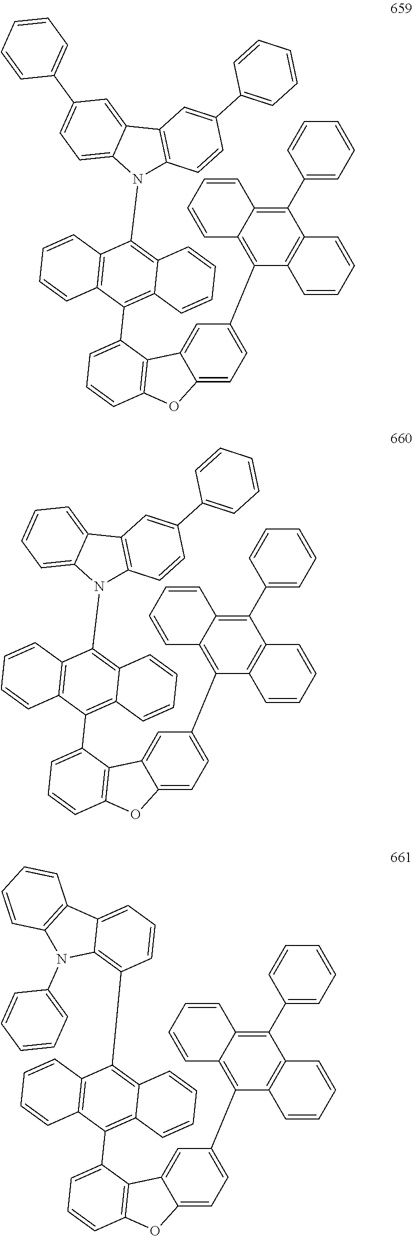 [SMILES: c1ccc(-c2ccc3c(c2)c2cc(-c4ccccc4)ccc2n3-c2c3ccccc3c(-c3cccc4oc5ccc(-c6c7ccccc7c(-c7ccccc7)c7ccccc67)cc5c34)c3ccccc23)cc1 ; c1ccc(-c2ccc3c(c2)c2ccccc2n3-c2c3ccccc3c(-c3cccc4oc5ccc(-c6c7ccccc7c(-c7ccccc7)c7ccccc67)cc5c34)c3ccccc23)cc1 ; c1ccc(-c2c3ccccc3c(-c3ccc4oc5cccc(-c6c7ccccc7c(-c7cccc8c9ccccc9n(-c9ccccc9)c78)c7ccccc67)c5c4c3)c3ccccc23)cc1]

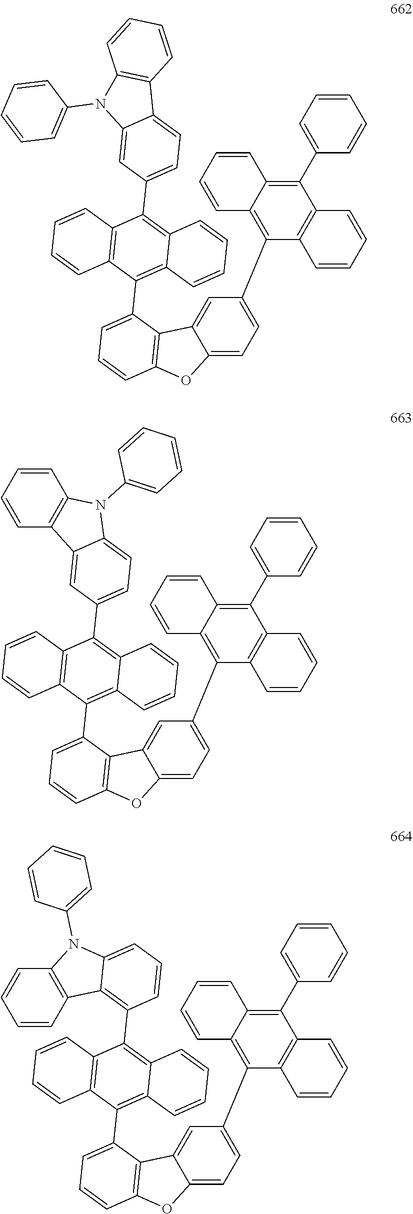 [SMILES: c1ccc(-c2c3ccccc3c(-c3ccc4oc5cccc(-c6c7ccccc7c(-c7ccc8c9ccccc9n(-c9ccccc9)c8c7)c7ccccc67)c5c4c3)c3ccccc23)cc1 ; c1ccc(-c2c3ccccc3c(-c3ccc4oc5cccc(-c6c7ccccc7c(-c7ccc8c(c7)c7ccccc7n8-c7ccccc7)c7ccccc67)c5c4c3)c3ccccc23)cc1 ; c1ccc(-c2c3ccccc3c(-c3ccc4oc5cccc(-c6c7ccccc7c(-c7cccc8c7c7ccccc7n8-c7ccccc7)c7ccccc67)c5c4c3)c3ccccc23)cc1]

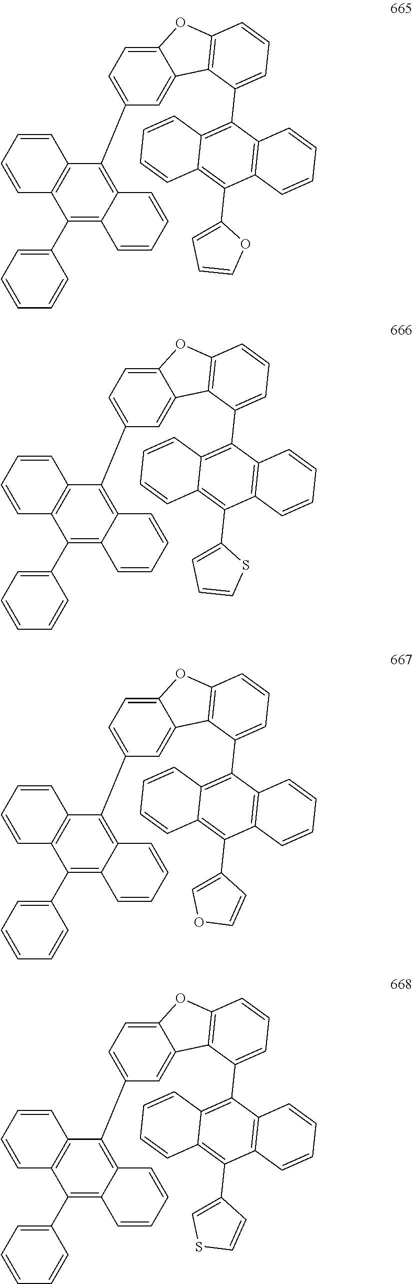 [SMILES: c1ccc(-c2c3ccccc3c(-c3ccc4oc5cccc(-c6c7ccccc7c(-c7ccco7)c7ccccc67)c5c4c3)c3ccccc23)cc1 ; c1ccc(-c2c3ccccc3c(-c3ccc4oc5cccc(-c6c7ccccc7c(-c7cccs7)c7ccccc67)c5c4c3)c3ccccc23)cc1 ; c1ccc(-c2c3ccccc3c(-c3ccc4oc5cccc(-c6c7ccccc7c(-c7ccoc7)c7ccccc67)c5c4c3)c3ccccc23)cc1 ; c1ccc(-c2c3ccccc3c(-c3ccc4oc5cccc(-c6c7ccccc7c(-c7ccsc7)c7ccccc67)c5c4c3)c3ccccc23)cc1]

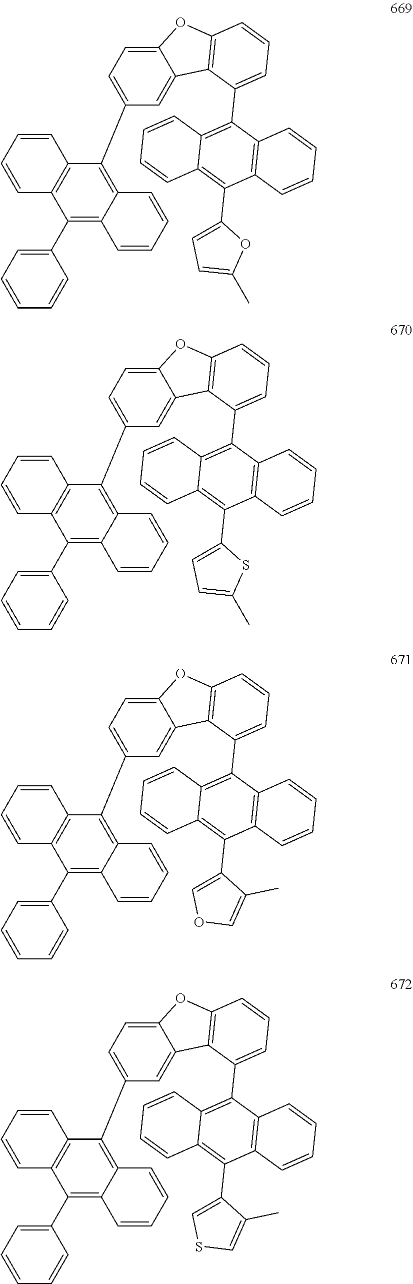

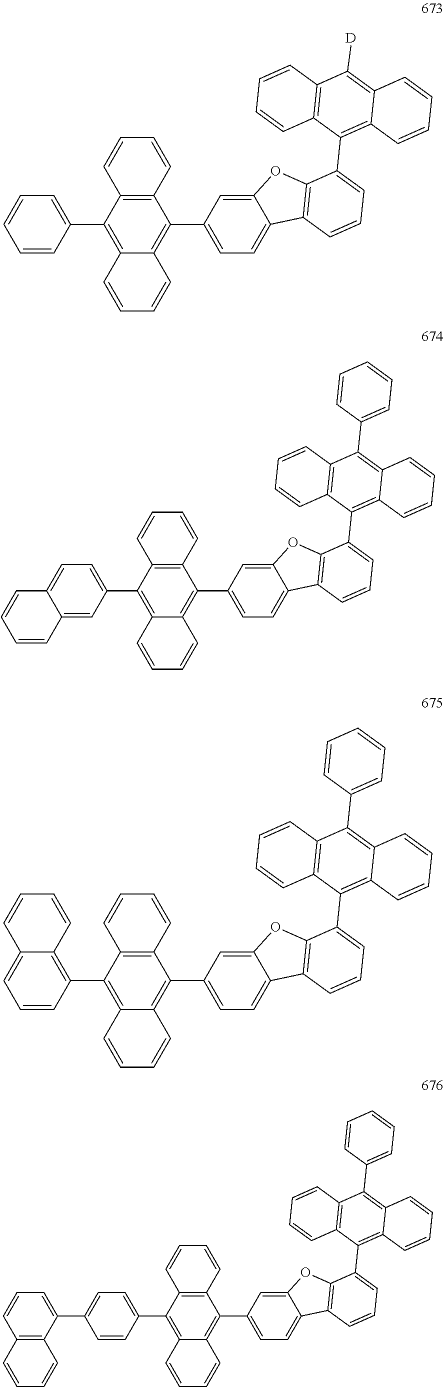 [SMILES: [2H]c1c2ccccc2c(-c2cccc3c2oc2cc(-c4c5ccccc5c(-c5ccccc5)c5ccccc45)ccc23)c2ccccc12 ; c1ccc(-c2c3ccccc3c(-c3cccc4c3oc3cc(-c5c6ccccc6c(-c6ccc7ccccc7c6)c6ccccc56)ccc34)c3ccccc23)cc1 ; c1ccc(-c2c3ccccc3c(-c3cccc4c3oc3cc(-c5c6ccccc6c(-c6cccc7ccccc67)c6ccccc56)ccc34)c3ccccc23)cc1 ; c1ccc(-c2c3ccccc3c(-c3cccc4c3oc3cc(-c5c6ccccc6c(-c6ccc(-c7cccc8ccccc78)cc6)c6ccccc56)ccc34)c3ccccc23)cc1]

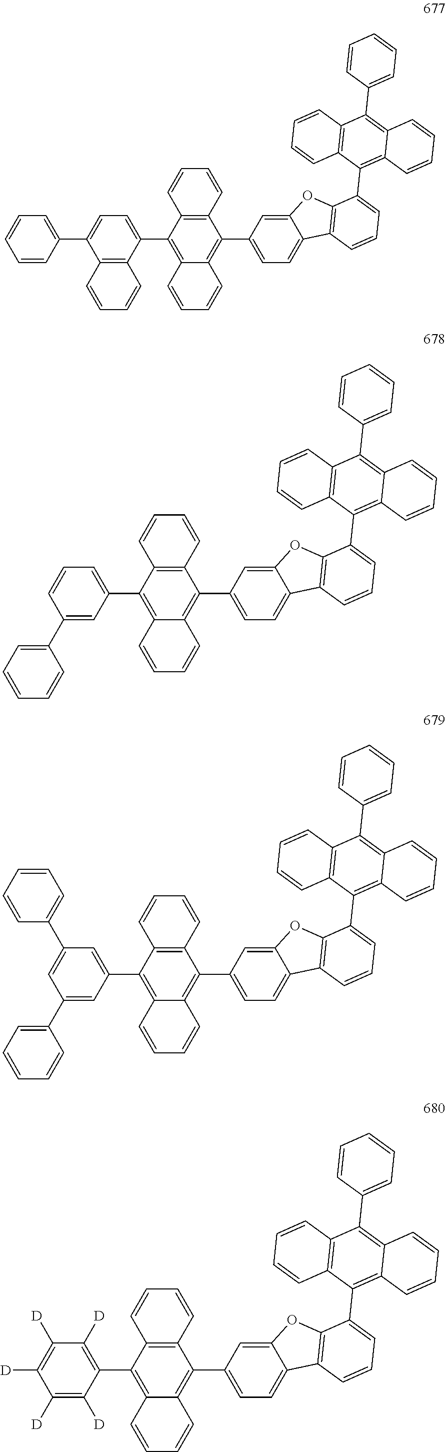 [SMILES: c1ccc(-c2ccc(-c3c4ccccc4c(-c4ccc5c(c4)oc4c(-c6c7ccccc7c(-c7ccccc7)c7ccccc67)cccc45)c4ccccc34)c3ccccc23)cc1 ; c1ccc(-c2cccc(-c3c4ccccc4c(-c4ccc5c(c4)oc4c(-c6c7ccccc7c(-c7ccccc7)c7ccccc67)cccc45)c4ccccc34)c2)cc1 ; c1ccc(-c2cc(-c3ccccc3)cc(-c3c4ccccc4c(-c4ccc5c(c4)oc4c(-c6c7ccccc7c(-c7ccccc7)c7ccccc67)cccc45)c4ccccc34)c2)cc1 ; [2H]c1c([2H])c([2H])c(-c2c3ccccc3c(-c3ccc4c(c3)oc3c(-c5c6ccccc6c(-c6ccccc6)c6ccccc56)cccc34)c3ccccc23)c([2H])c1[2H]]

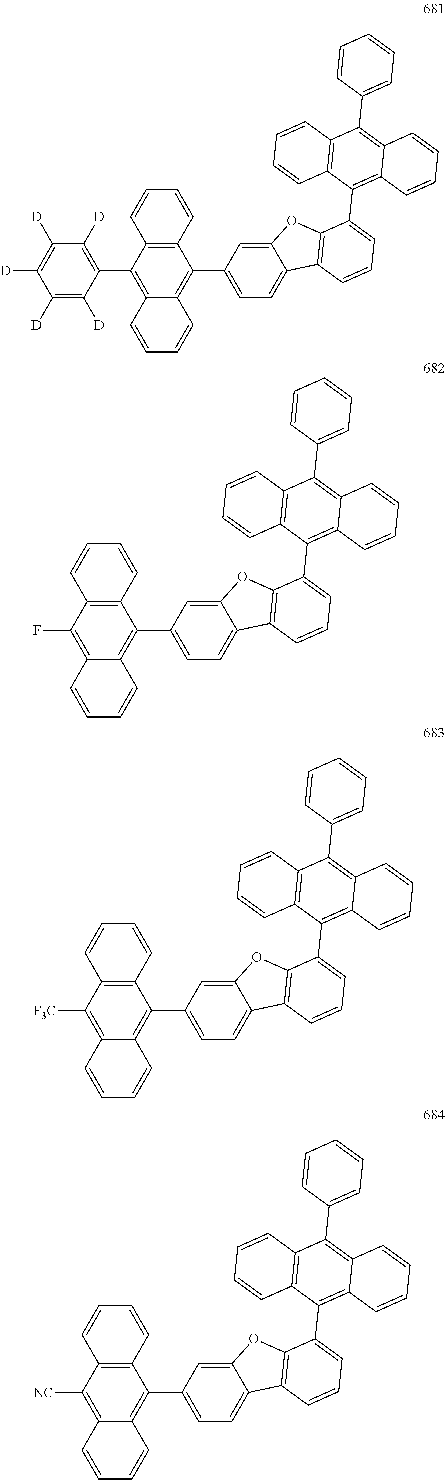 [SMILES: [2H]c1c([2H])c([2H])c(-c2c3ccccc3c(-c3ccc4c(c3)oc3c(-c5c6ccccc6c(-c6ccccc6)c6ccccc56)cccc34)c3ccccc23)c([2H])c1[2H] ; Fc1c2ccccc2c(-c2ccc3c(c2)oc2c(-c4c5ccccc5c(-c5ccccc5)c5ccccc45)cccc23)c2ccccc12 ; FC(F)(F)c1c2ccccc2c(-c2ccc3c(c2)oc2c(-c4c5ccccc5c(-c5ccccc5)c5ccccc45)cccc23)c2ccccc12 ; N#Cc1c2ccccc2c(-c2ccc3c(c2)oc2c(-c4c5ccccc5c(-c5ccccc5)c5ccccc45)cccc23)c2ccccc12]

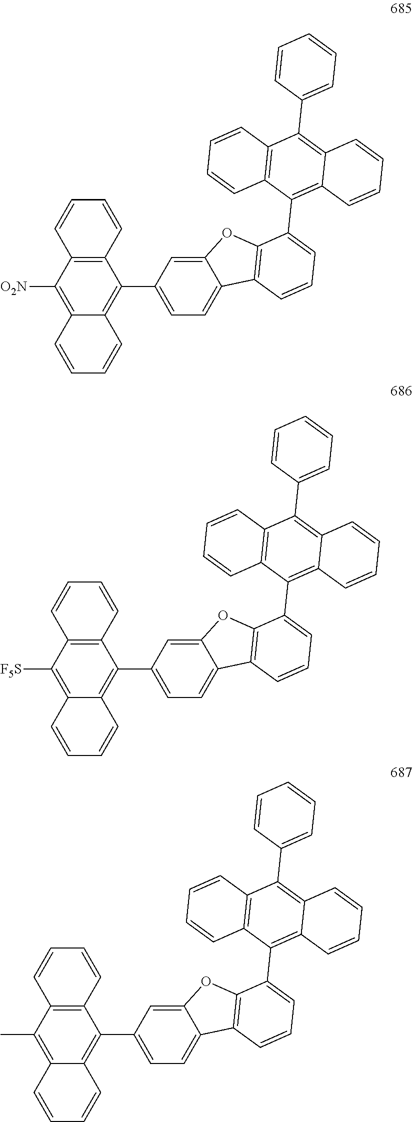

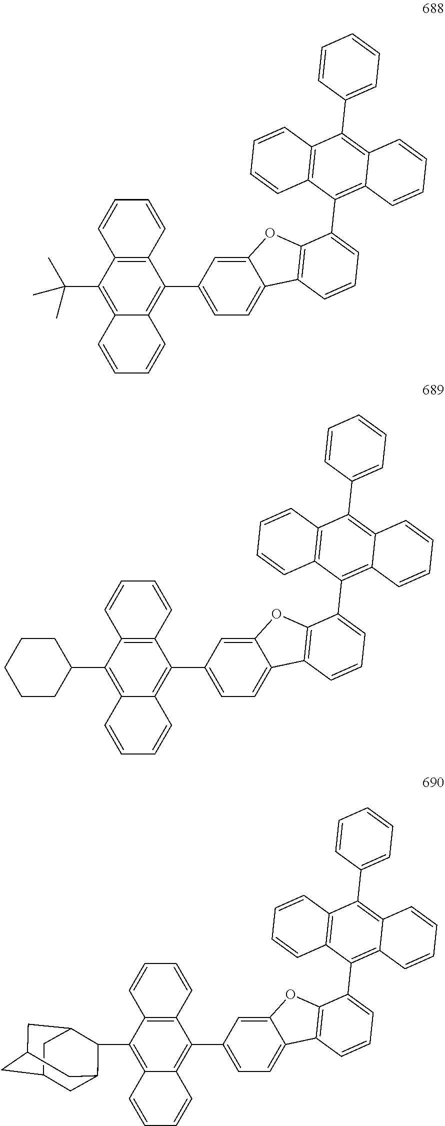 [SMILES: CC(C)(C)c1c2ccccc2c(-c2ccc3c(c2)oc2c(-c4c5ccccc5c(-c5ccccc5)c5ccccc45)cccc23)c2ccccc12 ; c1ccc(-c2c3ccccc3c(-c3cccc4c3oc3cc(-c5c6ccccc6c(C6CCCCC6)c6ccccc56)ccc34)c3ccccc23)cc1 ; c1ccc(-c2c3ccccc3c(-c3cccc4c3oc3cc(-c5c6ccccc6c(C6C7CC8CC(C7)CC6C8)c6ccccc56)ccc34)c3ccccc23)cc1]

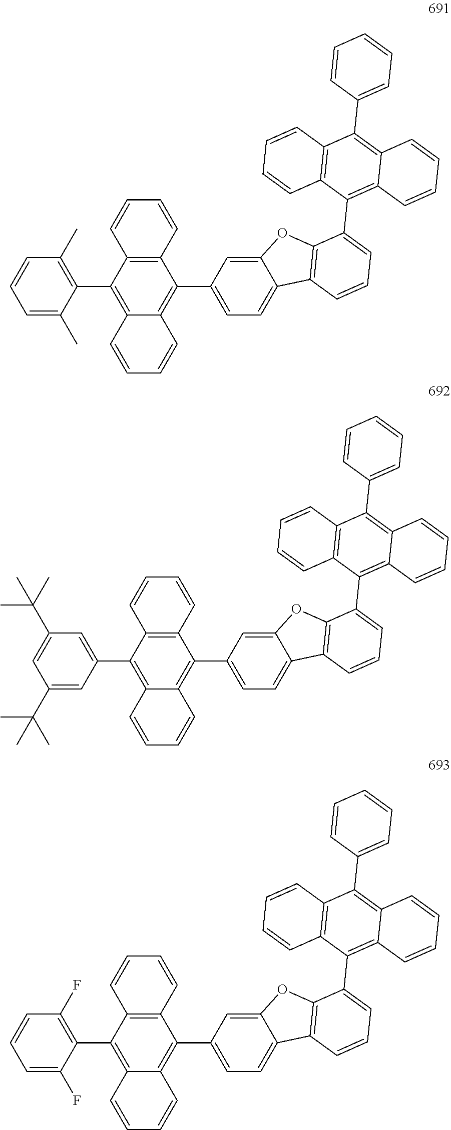 [SMILES: Cc1cccc(C)c1-c1c2ccccc2c(-c2ccc3c(c2)oc2c(-c4c5ccccc5c(-c5ccccc5)c5ccccc45)cccc23)c2ccccc12 ; CC(C)(C)c1cc(-c2c3ccccc3c(-c3ccc4c(c3)oc3c(-c5c6ccccc6c(-c6ccccc6)c6ccccc56)cccc34)c3ccccc23)cc(C(C)(C)C)c1 ; Fc1cccc(F)c1-c1c2ccccc2c(-c2ccc3c(c2)oc2c(-c4c5ccccc5c(-c5ccccc5)c5ccccc45)cccc23)c2ccccc12]

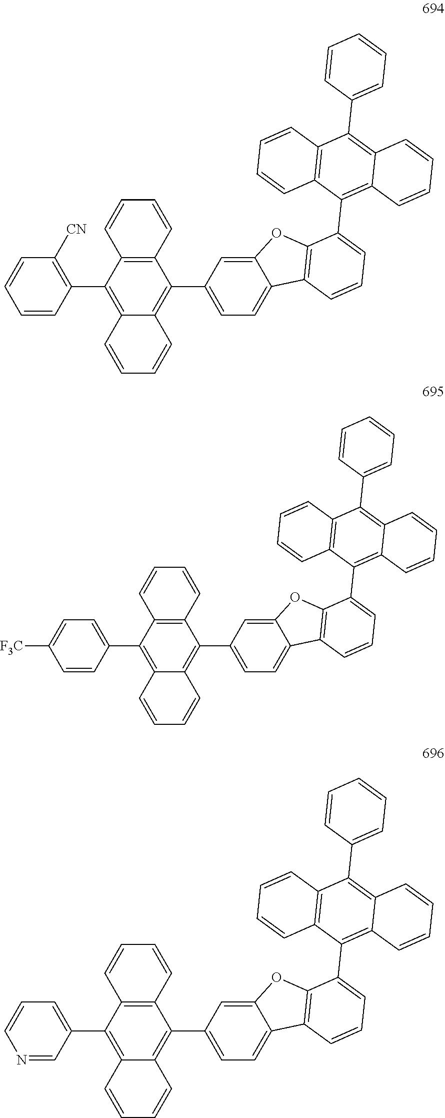 [SMILES: N#Cc1ccccc1-c1c2ccccc2c(-c2ccc3c(c2)oc2c(-c4c5ccccc5c(-c5ccccc5)c5ccccc45)cccc23)c2ccccc12 ; FC(F)(F)c1ccc(-c2c3ccccc3c(-c3ccc4c(c3)oc3c(-c5c6ccccc6c(-c6ccccc6)c6ccccc56)cccc34)c3ccccc23)cc1 ; c1ccc(-c2c3ccccc3c(-c3cccc4c3oc3cc(-c5c6ccccc6c(-c6cccnc6)c6ccccc56)ccc34)c3ccccc23)cc1]

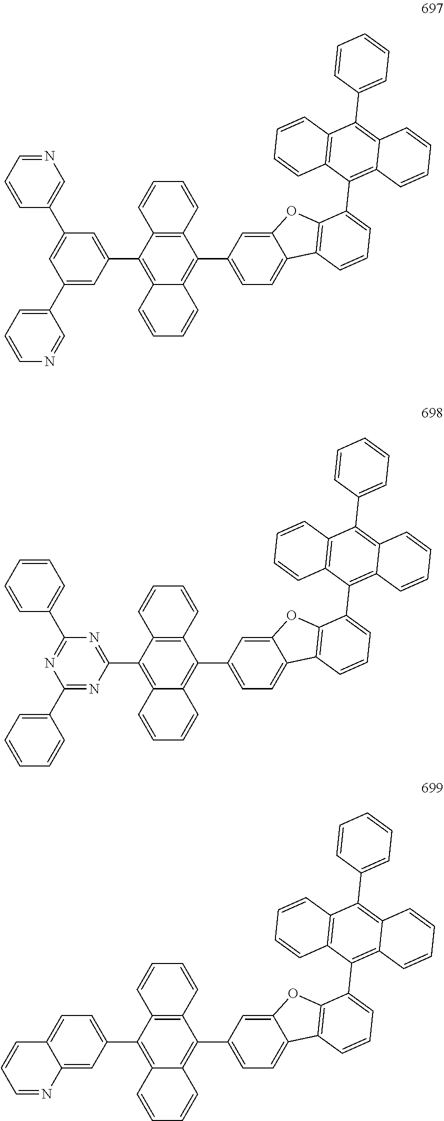 [SMILES: c1ccc(-c2c3ccccc3c(-c3cccc4c3oc3cc(-c5c6ccccc6c(-c6cc(-c7cccnc7)cc(-c7cccnc7)c6)c6ccccc56)ccc34)c3ccccc23)cc1 ; c1ccc(-c2nc(-c3ccccc3)nc(-c3c4ccccc4c(-c4ccc5c(c4)oc4c(-c6c7ccccc7c(-c7ccccc7)c7ccccc67)cccc45)c4ccccc34)n2)cc1 ; c1ccc(-c2c3ccccc3c(-c3cccc4c3oc3cc(-c5c6ccccc6c(-c6ccc7cccnc7c6)c6ccccc56)ccc34)c3ccccc23)cc1]

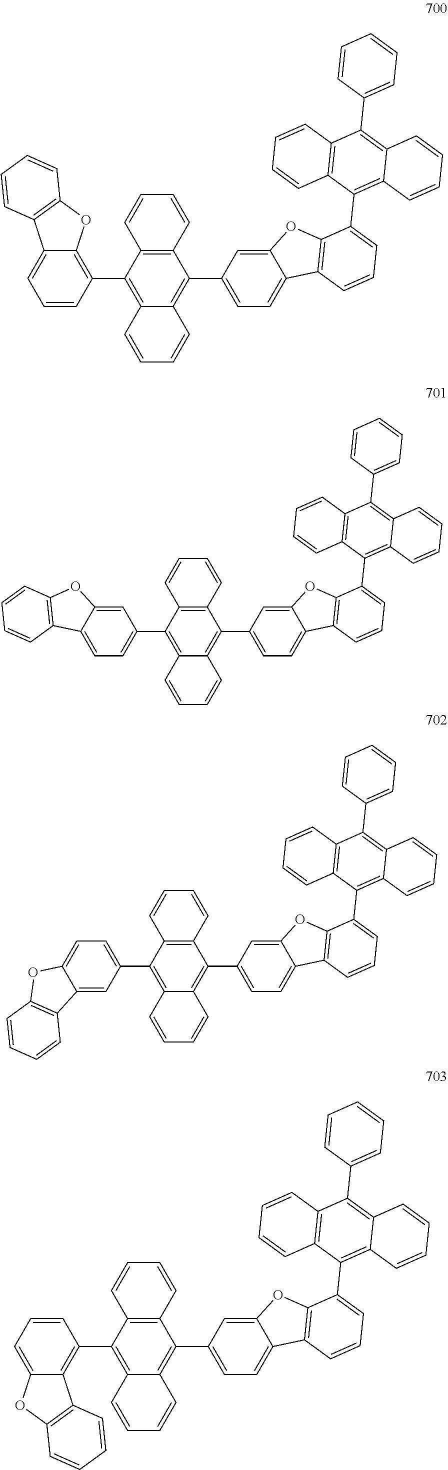 [SMILES: c1ccc(-c2c3ccccc3c(-c3cccc4c3oc3cc(-c5c6ccccc6c(-c6cccc7c6oc6ccccc67)c6ccccc56)ccc34)c3ccccc23)cc1 ; c1ccc(-c2c3ccccc3c(-c3cccc4c3oc3cc(-c5c6ccccc6c(-c6ccc7c(c6)oc6ccccc67)c6ccccc56)ccc34)c3ccccc23)cc1 ; c1ccc(-c2c3ccccc3c(-c3cccc4c3oc3cc(-c5c6ccccc6c(-c6ccc7oc8ccccc8c7c6)c6ccccc56)ccc34)c3ccccc23)cc1 ; c1ccc(-c2c3ccccc3c(-c3cccc4c3oc3cc(-c5c6ccccc6c(-c6cccc7oc8ccccc8c67)c6ccccc56)ccc34)c3ccccc23)cc1]

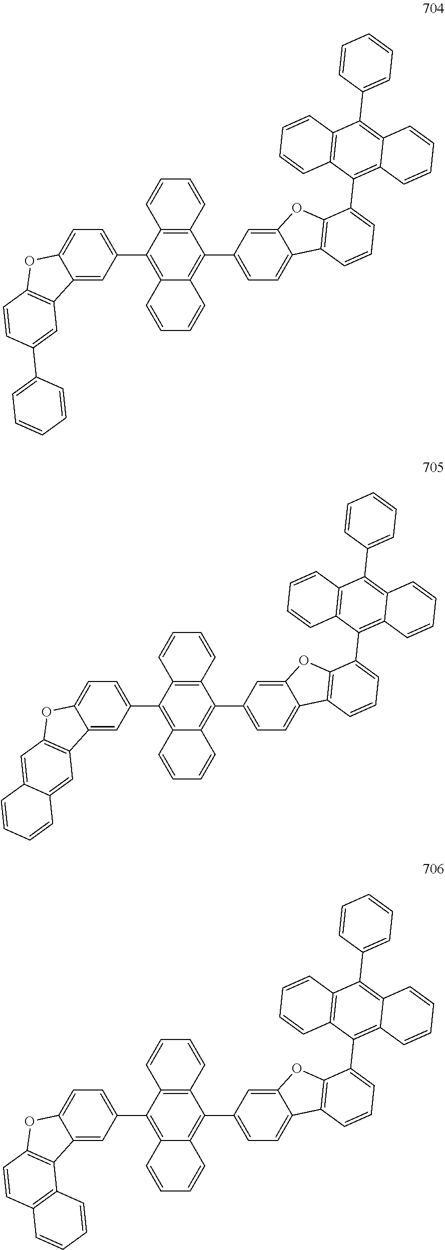 [SMILES: c1ccc(-c2ccc3oc4ccc(-c5c6ccccc6c(-c6ccc7c(c6)oc6c(-c8c9ccccc9c(-c9ccccc9)c9ccccc89)cccc67)c6ccccc56)cc4c3c2)cc1 ; c1ccc(-c2c3ccccc3c(-c3cccc4c3oc3cc(-c5c6ccccc6c(-c6ccc7oc8cc9ccccc9cc8c7c6)c6ccccc56)ccc34)c3ccccc23)cc1 ; c1ccc(-c2c3ccccc3c(-c3cccc4c3oc3cc(-c5c6ccccc6c(-c6ccc7oc8ccc9ccccc9c8c7c6)c6ccccc56)ccc34)c3ccccc23)cc1]

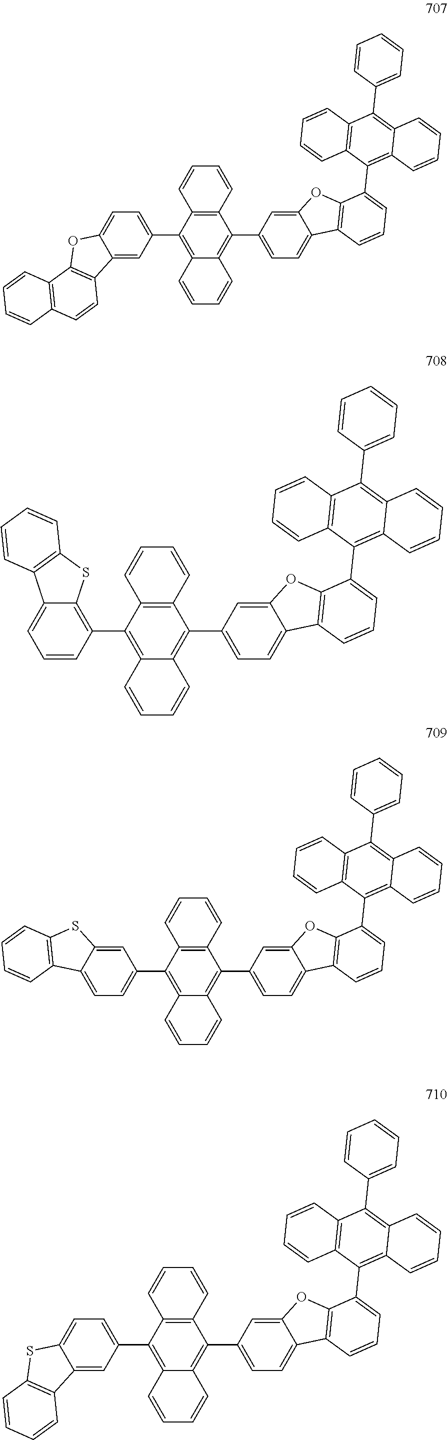 [SMILES: c1ccc(-c2c3ccccc3c(-c3cccc4c3oc3cc(-c5c6ccccc6c(-c6ccc7oc8c9ccccc9ccc8c7c6)c6ccccc56)ccc34)c3ccccc23)cc1 ; c1ccc(-c2c3ccccc3c(-c3cccc4c3oc3cc(-c5c6ccccc6c(-c6cccc7c6sc6ccccc67)c6ccccc56)ccc34)c3ccccc23)cc1 ; c1ccc(-c2c3ccccc3c(-c3cccc4c3oc3cc(-c5c6ccccc6c(-c6ccc7c(c6)sc6ccccc67)c6ccccc56)ccc34)c3ccccc23)cc1 ; c1ccc(-c2c3ccccc3c(-c3cccc4c3oc3cc(-c5c6ccccc6c(-c6ccc7sc8ccccc8c7c6)c6ccccc56)ccc34)c3ccccc23)cc1]

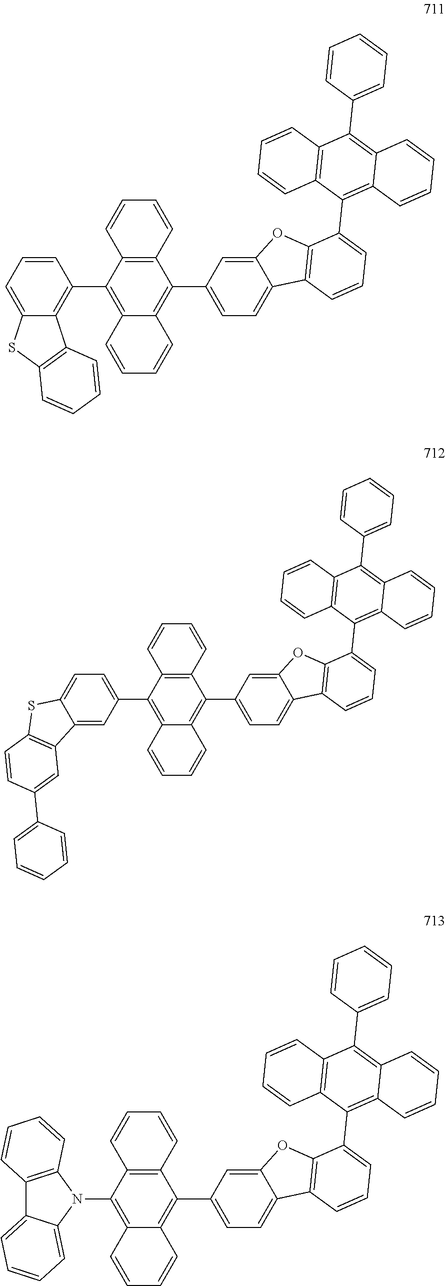 [SMILES: c1ccc(-c2c3ccccc3c(-c3cccc4c3oc3cc(-c5c6ccccc6c(-c6cccc7sc8ccccc8c67)c6ccccc56)ccc34)c3ccccc23)cc1 ; c1ccc(-c2ccc3sc4ccc(-c5c6ccccc6c(-c6ccc7c(c6)oc6c(-c8c9ccccc9c(-c9ccccc9)c9ccccc89)cccc67)c6ccccc56)cc4c3c2)cc1 ; c1ccc(-c2c3ccccc3c(-c3cccc4c3oc3cc(-c5c6ccccc6c(-n6c7ccccc7c7ccccc76)c6ccccc56)ccc34)c3ccccc23)cc1]

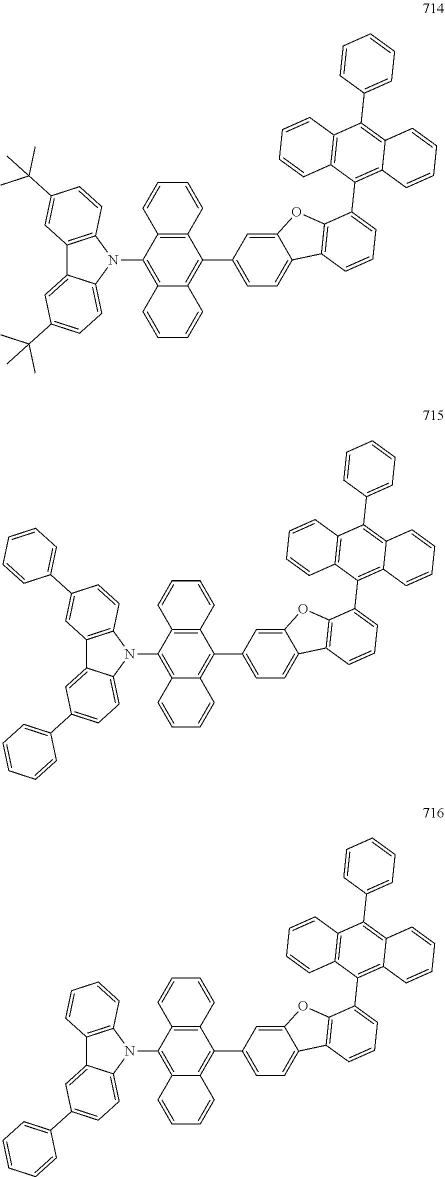 [SMILES: CC(C)(C)c1ccc2c(c1)c1cc(C(C)(C)C)ccc1n2-c1c2ccccc2c(-c2ccc3c(c2)oc2c(-c4c5ccccc5c(-c5ccccc5)c5ccccc45)cccc23)c2ccccc12 ; c1ccc(-c2ccc3c(c2)c2cc(-c4ccccc4)ccc2n3-c2c3ccccc3c(-c3ccc4c(c3)oc3c(-c5c6ccccc6c(-c6ccccc6)c6ccccc56)cccc34)c3ccccc23)cc1 ; c1ccc(-c2ccc3c(c2)c2ccccc2n3-c2c3ccccc3c(-c3ccc4c(c3)oc3c(-c5c6ccccc6c(-c6ccccc6)c6ccccc56)cccc34)c3ccccc23)cc1]

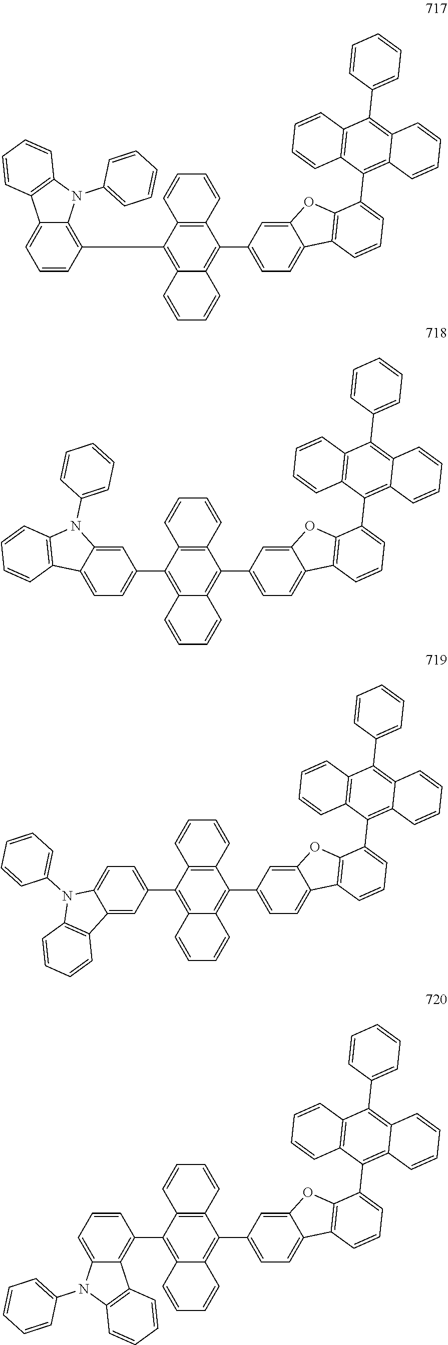 [SMILES: c1ccc(-c2c3ccccc3c(-c3cccc4c3oc3cc(-c5c6ccccc6c(-c6cccc7c8ccccc8n(-c8ccccc8)c67)c6ccccc56)ccc34)c3ccccc23)cc1 ; c1ccc(-c2c3ccccc3c(-c3cccc4c3oc3cc(-c5c6ccccc6c(-c6ccc7c8ccccc8n(-c8ccccc8)c7c6)c6ccccc56)ccc34)c3ccccc23)cc1 ; c1ccc(-c2c3ccccc3c(-c3cccc4c3oc3cc(-c5c6ccccc6c(-c6ccc7c(c6)c6ccccc6n7-c6ccccc6)c6ccccc56)ccc34)c3ccccc23)cc1 ; c1ccc(-c2c3ccccc3c(-c3cccc4c3oc3cc(-c5c6ccccc6c(-c6cccc7c6c6ccccc6n7-c6ccccc6)c6ccccc56)ccc34)c3ccccc23)cc1]

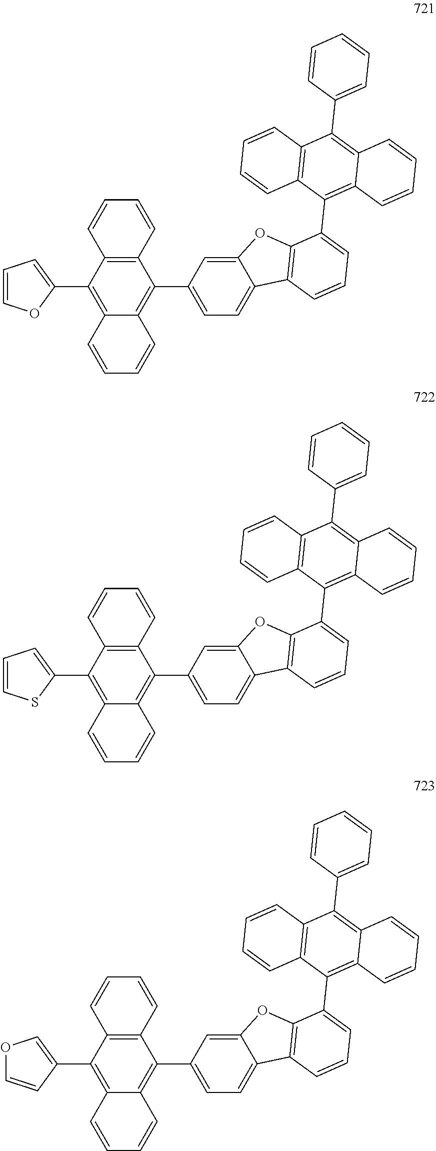 [SMILES: c1ccc(-c2c3ccccc3c(-c3cccc4c3oc3cc(-c5c6ccccc6c(-c6ccco6)c6ccccc56)ccc34)c3ccccc23)cc1 ; c1ccc(-c2c3ccccc3c(-c3cccc4c3oc3cc(-c5c6ccccc6c(-c6cccs6)c6ccccc56)ccc34)c3ccccc23)cc1 ; c1ccc(-c2c3ccccc3c(-c3cccc4c3oc3cc(-c5c6ccccc6c(-c6ccoc6)c6ccccc56)ccc34)c3ccccc23)cc1]

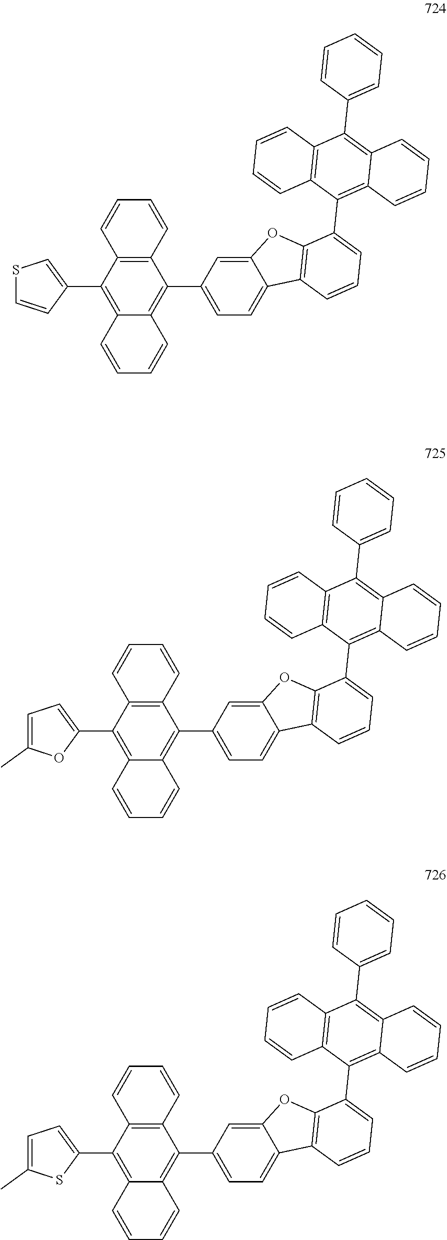 [SMILES: c1ccc(-c2c3ccccc3c(-c3cccc4c3oc3cc(-c5c6ccccc6c(-c6ccsc6)c6ccccc56)ccc34)c3ccccc23)cc1 ; Cc1ccc(-c2c3ccccc3c(-c3ccc4c(c3)oc3c(-c5c6ccccc6c(-c6ccccc6)c6ccccc56)cccc34)c3ccccc23)o1 ; Cc1ccc(-c2c3ccccc3c(-c3ccc4c(c3)oc3c(-c5c6ccccc6c(-c6ccccc6)c6ccccc56)cccc34)c3ccccc23)s1]

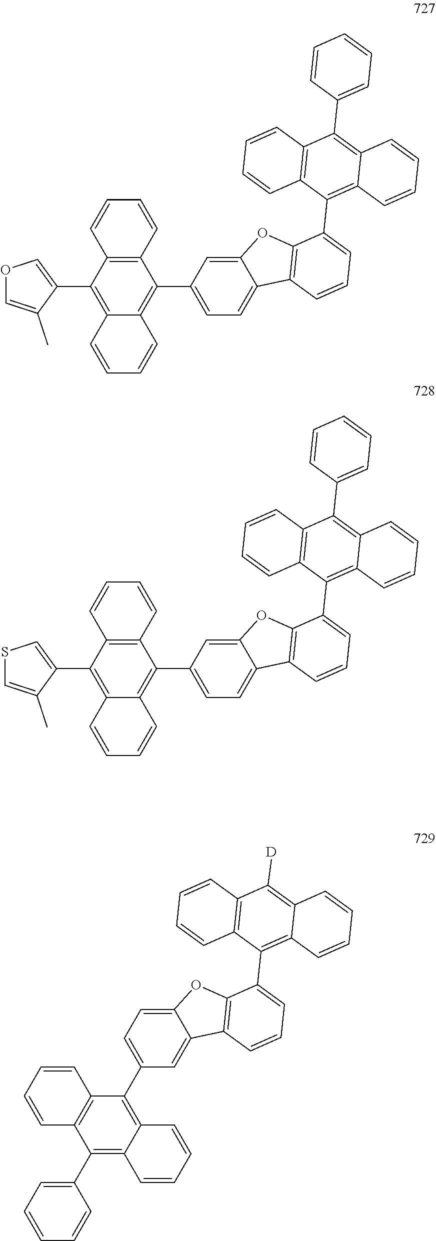 [SMILES: Cc1cocc1-c1c2ccccc2c(-c2ccc3c(c2)oc2c(-c4c5ccccc5c(-c5ccccc5)c5ccccc45)cccc23)c2ccccc12 ; Cc1cscc1-c1c2ccccc2c(-c2ccc3c(c2)oc2c(-c4c5ccccc5c(-c5ccccc5)c5ccccc45)cccc23)c2ccccc12 ; [2H]c1c2ccccc2c(-c2cccc3c2oc2ccc(-c4c5ccccc5c(-c5ccccc5)c5ccccc45)cc23)c2ccccc12]

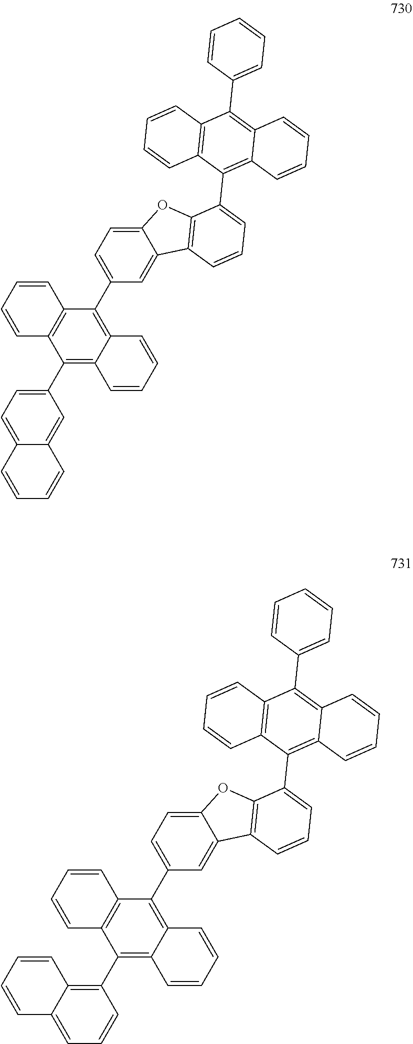 [SMILES: c1ccc(-c2c3ccccc3c(-c3cccc4c3oc3ccc(-c5c6ccccc6c(-c6ccc7ccccc7c6)c6ccccc56)cc34)c3ccccc23)cc1 ; c1ccc(-c2c3ccccc3c(-c3cccc4c3oc3ccc(-c5c6ccccc6c(-c6cccc7ccccc67)c6ccccc56)cc34)c3ccccc23)cc1]

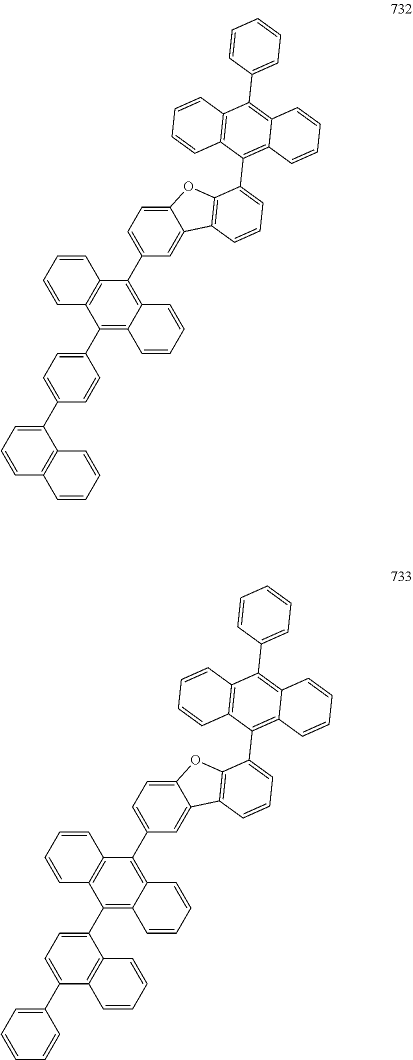 [SMILES: c1ccc(-c2c3ccccc3c(-c3cccc4c3oc3ccc(-c5c6ccccc6c(-c6ccc(-c7cccc8ccccc78)cc6)c6ccccc56)cc34)c3ccccc23)cc1 ; c1ccc(-c2ccc(-c3c4ccccc4c(-c4ccc5oc6c(-c7c8ccccc8c(-c8ccccc8)c8ccccc78)cccc6c5c4)c4ccccc34)c3ccccc23)cc1]

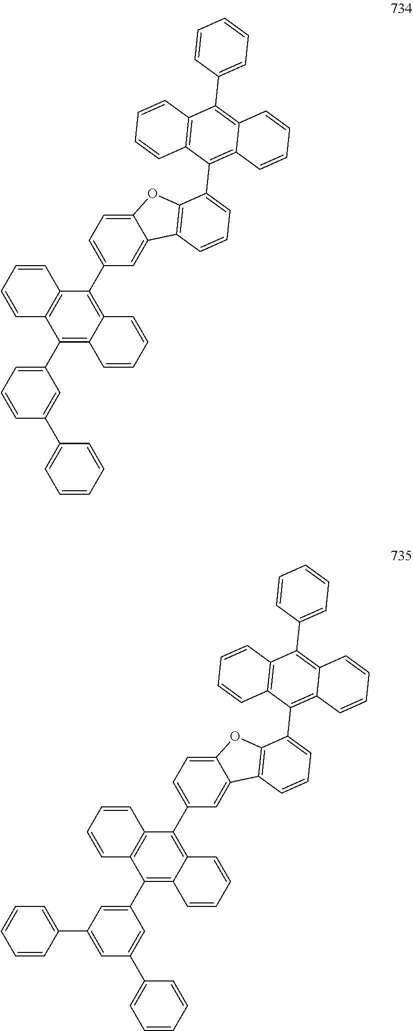 [SMILES: c1ccc(-c2cccc(-c3c4ccccc4c(-c4ccc5oc6c(-c7c8ccccc8c(-c8ccccc8)c8ccccc78)cccc6c5c4)c4ccccc34)c2)cc1 ; c1ccc(-c2cc(-c3ccccc3)cc(-c3c4ccccc4c(-c4ccc5oc6c(-c7c8ccccc8c(-c8ccccc8)c8ccccc78)cccc6c5c4)c4ccccc34)c2)cc1]

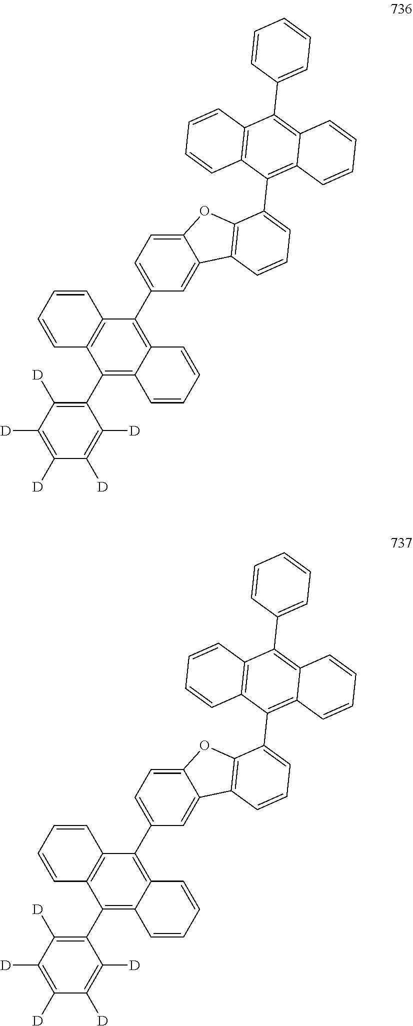 [SMILES: [2H]c1c([2H])c([2H])c(-c2c3ccccc3c(-c3ccc4oc5c(-c6c7ccccc7c(-c7ccccc7)c7ccccc67)cccc5c4c3)c3ccccc23)c([2H])c1[2H] ; [2H]c1c([2H])c([2H])c(-c2c3ccccc3c(-c3ccc4oc5c(-c6c7ccccc7c(-c7ccccc7)c7ccccc67)cccc5c4c3)c3ccccc23)c([2H])c1[2H]]

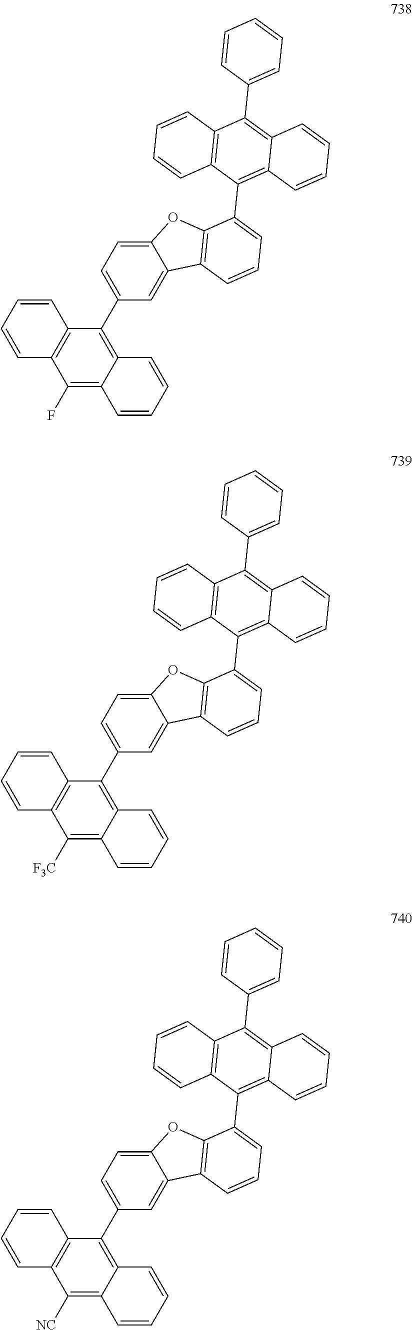 [SMILES: Fc1c2ccccc2c(-c2ccc3oc4c(-c5c6ccccc6c(-c6ccccc6)c6ccccc56)cccc4c3c2)c2ccccc12 ; FC(F)(F)c1c2ccccc2c(-c2ccc3oc4c(-c5c6ccccc6c(-c6ccccc6)c6ccccc56)cccc4c3c2)c2ccccc12 ; N#Cc1c2ccccc2c(-c2ccc3oc4c(-c5c6ccccc6c(-c6ccccc6)c6ccccc56)cccc4c3c2)c2ccccc12]

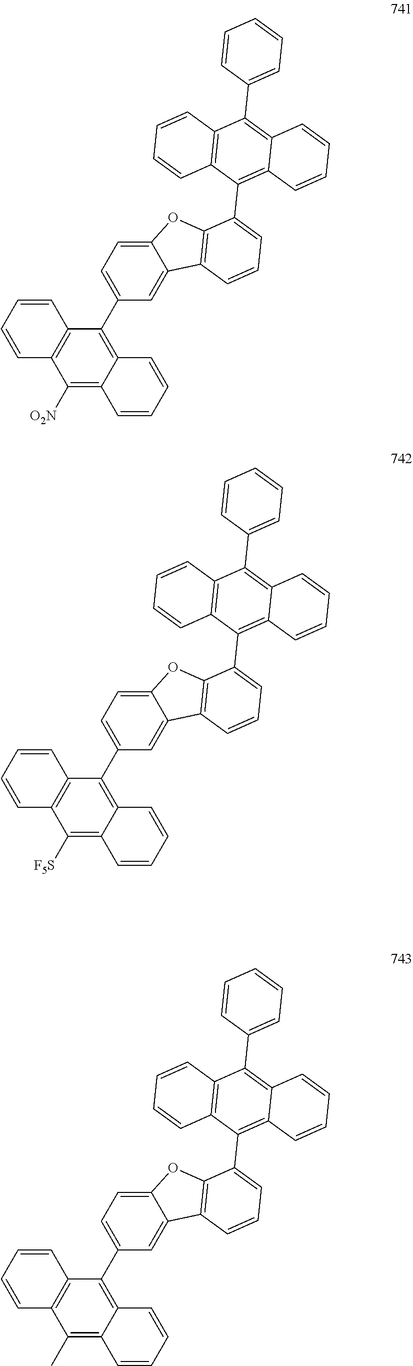

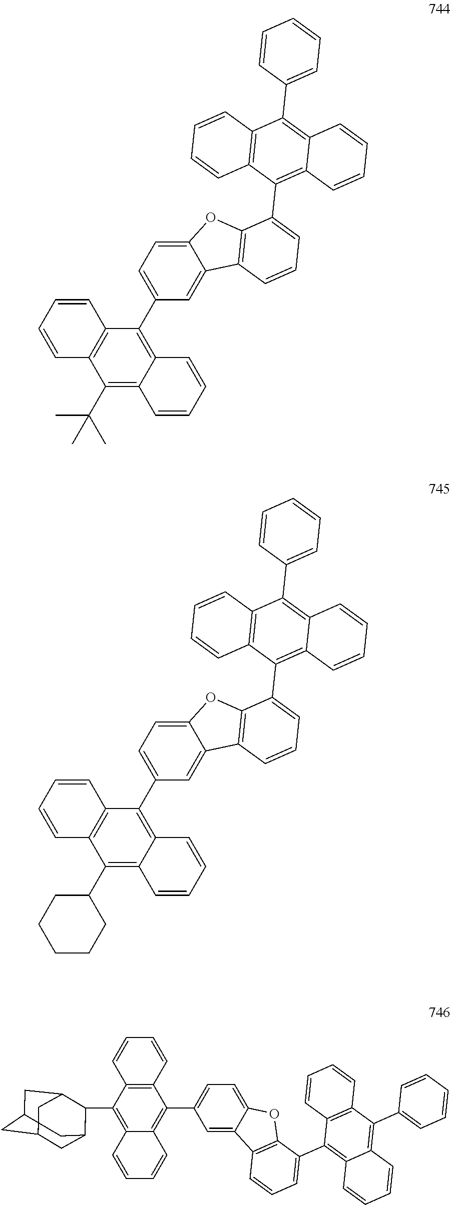 [SMILES: CC(C)(C)c1c2ccccc2c(-c2ccc3oc4c(-c5c6ccccc6c(-c6ccccc6)c6ccccc56)cccc4c3c2)c2ccccc12 ; c1ccc(-c2c3ccccc3c(-c3cccc4c3oc3ccc(-c5c6ccccc6c(C6CCCCC6)c6ccccc56)cc34)c3ccccc23)cc1 ; c1ccc(-c2c3ccccc3c(-c3cccc4c3oc3ccc(-c5c6ccccc6c(C6C7CC8CC(C7)CC6C8)c6ccccc56)cc34)c3ccccc23)cc1]

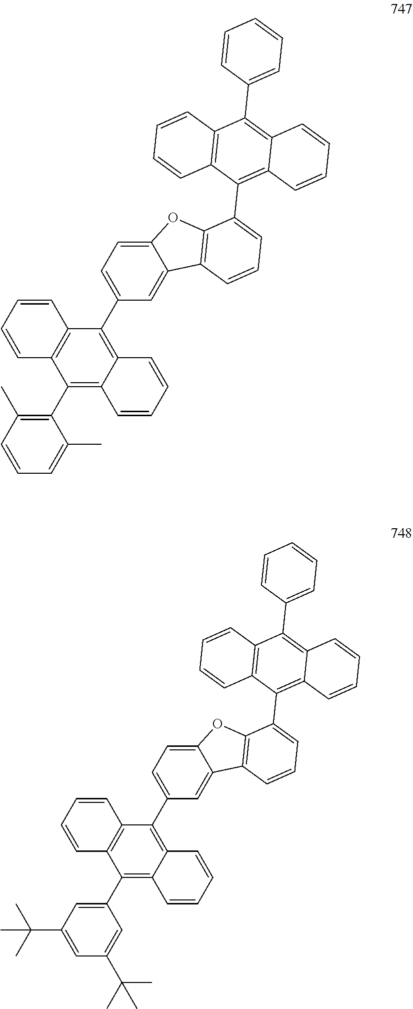 [SMILES: Cc1cccc(C)c1-c1c2ccccc2c(-c2ccc3oc4c(-c5c6ccccc6c(-c6ccccc6)c6ccccc56)cccc4c3c2)c2ccccc12 ; CC(C)(C)c1cc(-c2c3ccccc3c(-c3ccc4oc5c(-c6c7ccccc7c(-c7ccccc7)c7ccccc67)cccc5c4c3)c3ccccc23)cc(C(C)(C)C)c1]

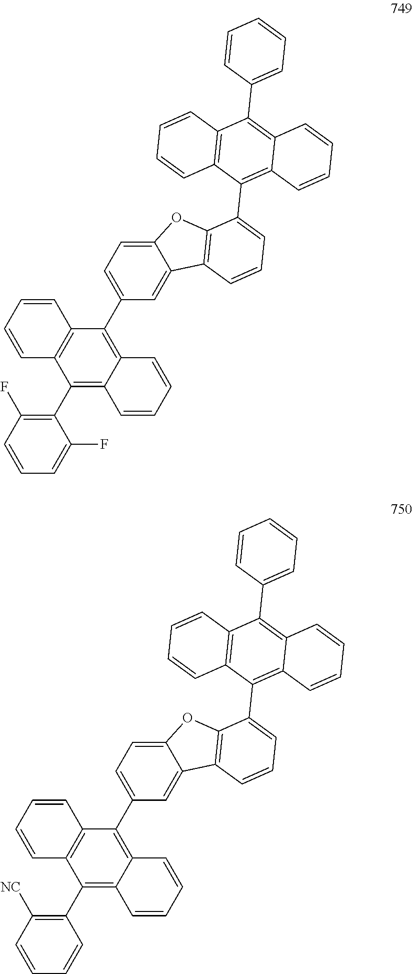 [SMILES: Fc1cccc(F)c1-c1c2ccccc2c(-c2ccc3oc4c(-c5c6ccccc6c(-c6ccccc6)c6ccccc56)cccc4c3c2)c2ccccc12 ; N#Cc1ccccc1-c1c2ccccc2c(-c2ccc3oc4c(-c5c6ccccc6c(-c6ccccc6)c6ccccc56)cccc4c3c2)c2ccccc12]

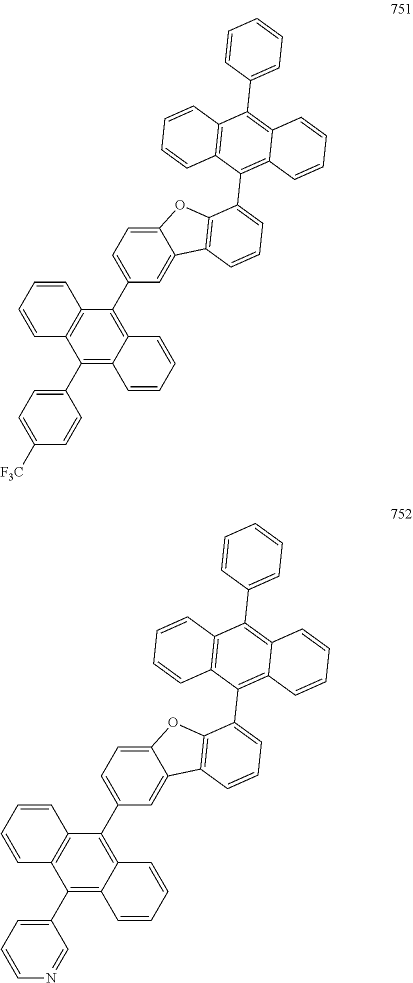 [SMILES: FC(F)(F)c1ccc(-c2c3ccccc3c(-c3ccc4oc5c(-c6c7ccccc7c(-c7ccccc7)c7ccccc67)cccc5c4c3)c3ccccc23)cc1 ; c1ccc(-c2c3ccccc3c(-c3cccc4c3oc3ccc(-c5c6ccccc6c(-c6cccnc6)c6ccccc56)cc34)c3ccccc23)cc1]

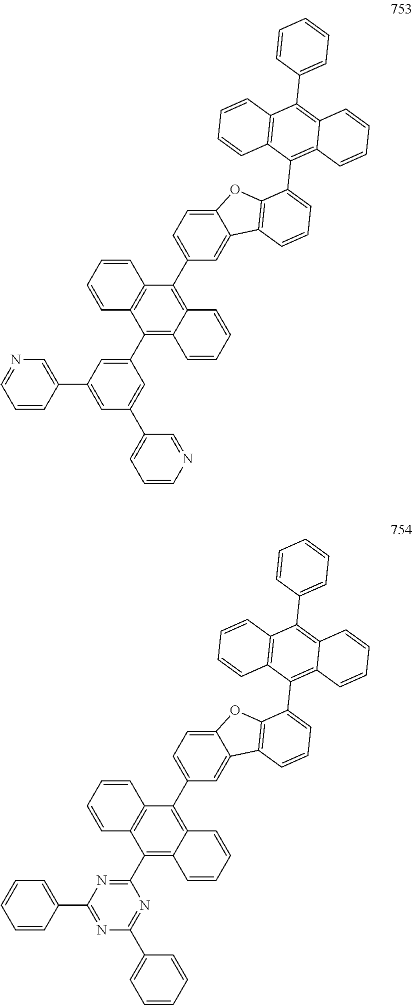 [SMILES: c1ccc(-c2c3ccccc3c(-c3cccc4c3oc3ccc(-c5c6ccccc6c(-c6cc(-c7cccnc7)cc(-c7cccnc7)c6)c6ccccc56)cc34)c3ccccc23)cc1 ; c1ccc(-c2nc(-c3ccccc3)nc(-c3c4ccccc4c(-c4ccc5oc6c(-c7c8ccccc8c(-c8ccccc8)c8ccccc78)cccc6c5c4)c4ccccc34)n2)cc1]

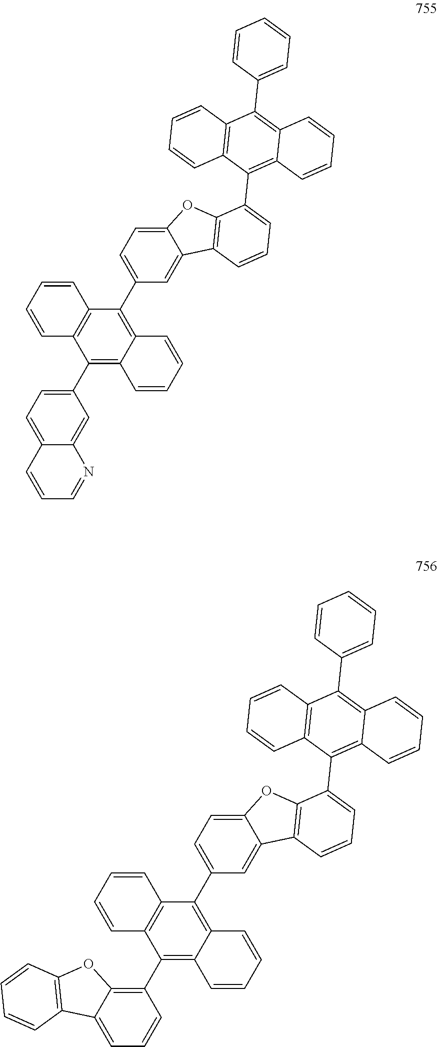 [SMILES: c1ccc(-c2c3ccccc3c(-c3cccc4c3oc3ccc(-c5c6ccccc6c(-c6ccc7cccnc7c6)c6ccccc56)cc34)c3ccccc23)cc1 ; c1ccc(-c2c3ccccc3c(-c3cccc4c3oc3ccc(-c5c6ccccc6c(-c6cccc7c6oc6ccccc67)c6ccccc56)cc34)c3ccccc23)cc1]

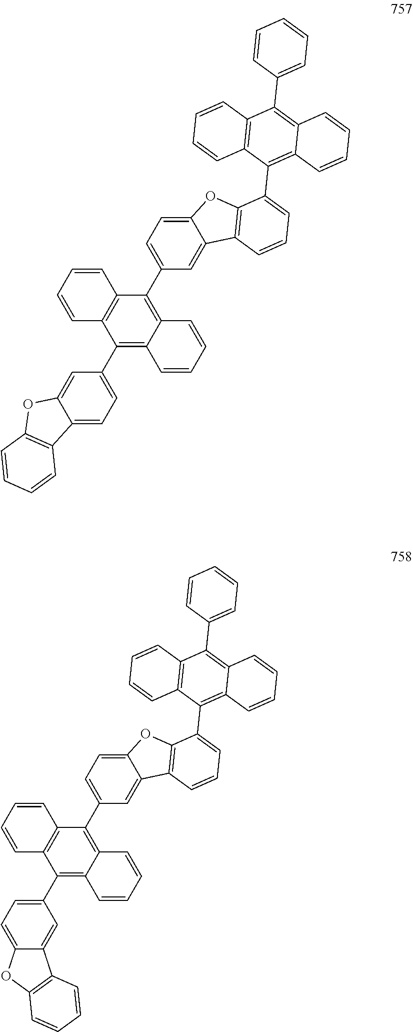 [SMILES: c1ccc(-c2c3ccccc3c(-c3cccc4c3oc3ccc(-c5c6ccccc6c(-c6ccc7c(c6)oc6ccccc67)c6ccccc56)cc34)c3ccccc23)cc1 ; c1ccc(-c2c3ccccc3c(-c3cccc4c3oc3ccc(-c5c6ccccc6c(-c6ccc7oc8ccccc8c7c6)c6ccccc56)cc34)c3ccccc23)cc1]

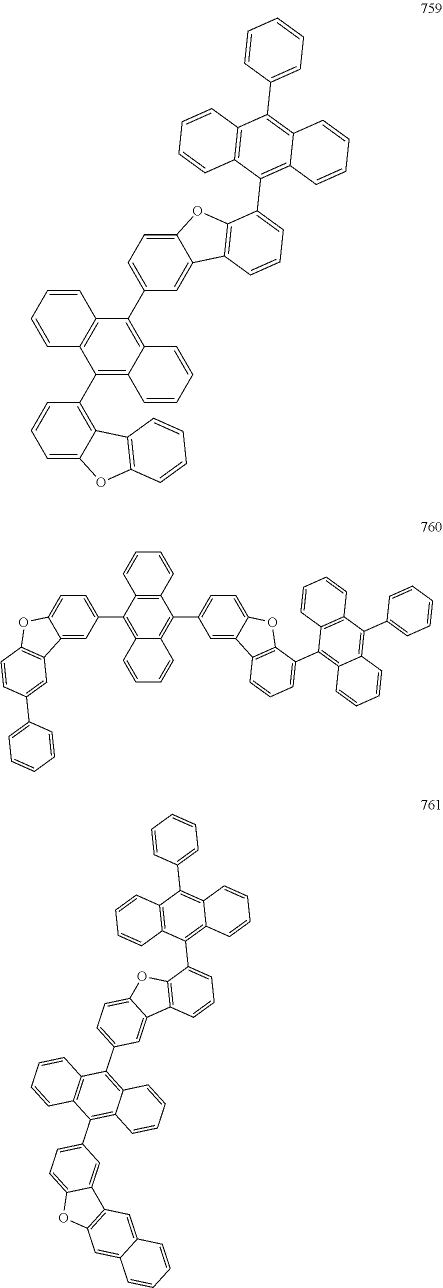 [SMILES: c1ccc(-c2c3ccccc3c(-c3cccc4c3oc3ccc(-c5c6ccccc6c(-c6cccc7oc8ccccc8c67)c6ccccc56)cc34)c3ccccc23)cc1 ; c1ccc(-c2ccc3oc4ccc(-c5c6ccccc6c(-c6ccc7oc8c(-c9c%10ccccc%10c(-c%10ccccc%10)c%10ccccc9%10)cccc8c7c6)c6ccccc56)cc4c3c2)cc1 ; c1ccc(-c2c3ccccc3c(-c3cccc4c3oc3ccc(-c5c6ccccc6c(-c6ccc7oc8cc9ccccc9cc8c7c6)c6ccccc56)cc34)c3ccccc23)cc1]

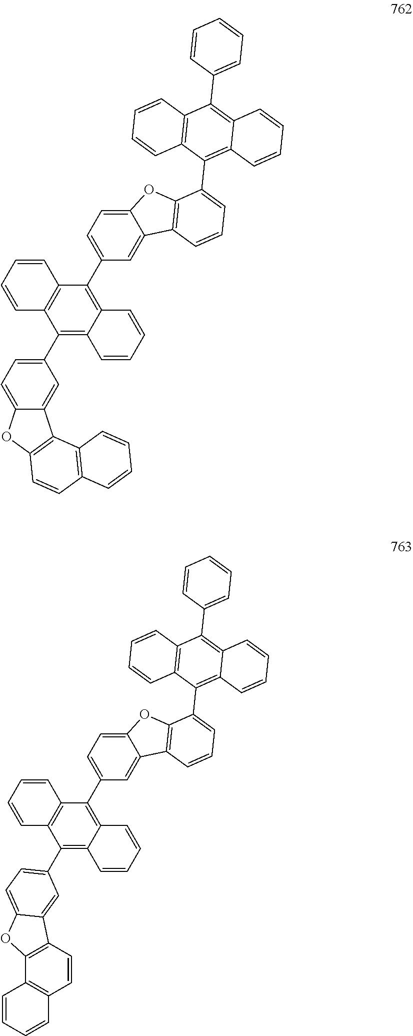 [SMILES: c1ccc(-c2c3ccccc3c(-c3cccc4c3oc3ccc(-c5c6ccccc6c(-c6ccc7oc8ccc9ccccc9c8c7c6)c6ccccc56)cc34)c3ccccc23)cc1 ; c1ccc(-c2c3ccccc3c(-c3cccc4c3oc3ccc(-c5c6ccccc6c(-c6ccc7oc8c9ccccc9ccc8c7c6)c6ccccc56)cc34)c3ccccc23)cc1]

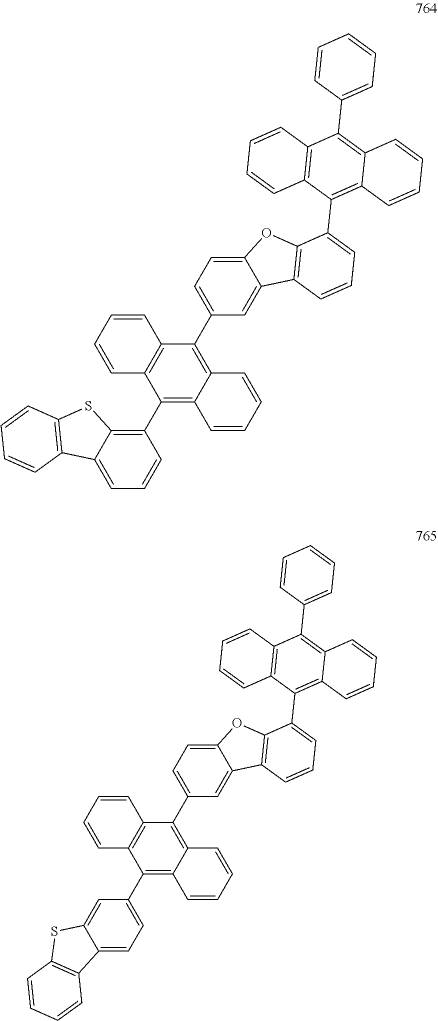 [SMILES: c1ccc(-c2c3ccccc3c(-c3cccc4c3oc3ccc(-c5c6ccccc6c(-c6cccc7c6sc6ccccc67)c6ccccc56)cc34)c3ccccc23)cc1 ; c1ccc(-c2c3ccccc3c(-c3cccc4c3oc3ccc(-c5c6ccccc6c(-c6ccc7c(c6)sc6ccccc67)c6ccccc56)cc34)c3ccccc23)cc1]

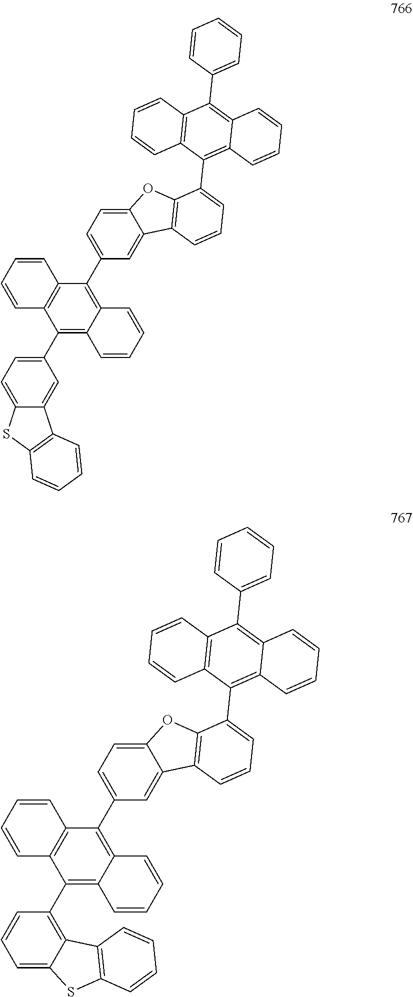 [SMILES: c1ccc(-c2c3ccccc3c(-c3cccc4c3oc3ccc(-c5c6ccccc6c(-c6ccc7sc8ccccc8c7c6)c6ccccc56)cc34)c3ccccc23)cc1 ; c1ccc(-c2c3ccccc3c(-c3cccc4c3oc3ccc(-c5c6ccccc6c(-c6cccc7sc8ccccc8c67)c6ccccc56)cc34)c3ccccc23)cc1]

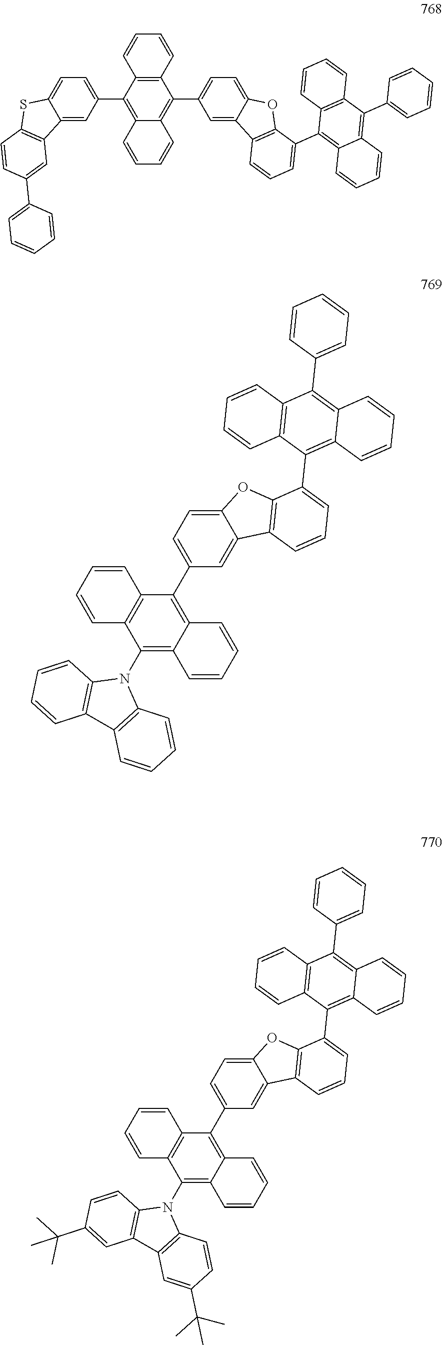 [SMILES: c1ccc(-c2ccc3sc4ccc(-c5c6ccccc6c(-c6ccc7oc8c(-c9c%10ccccc%10c(-c%10ccccc%10)c%10ccccc9%10)cccc8c7c6)c6ccccc56)cc4c3c2)cc1 ; c1ccc(-c2c3ccccc3c(-c3cccc4c3oc3ccc(-c5c6ccccc6c(-n6c7ccccc7c7ccccc76)c6ccccc56)cc34)c3ccccc23)cc1 ; CC(C)(C)c1ccc2c(c1)c1cc(C(C)(C)C)ccc1n2-c1c2ccccc2c(-c2ccc3oc4c(-c5c6ccccc6c(-c6ccccc6)c6ccccc56)cccc4c3c2)c2ccccc12]

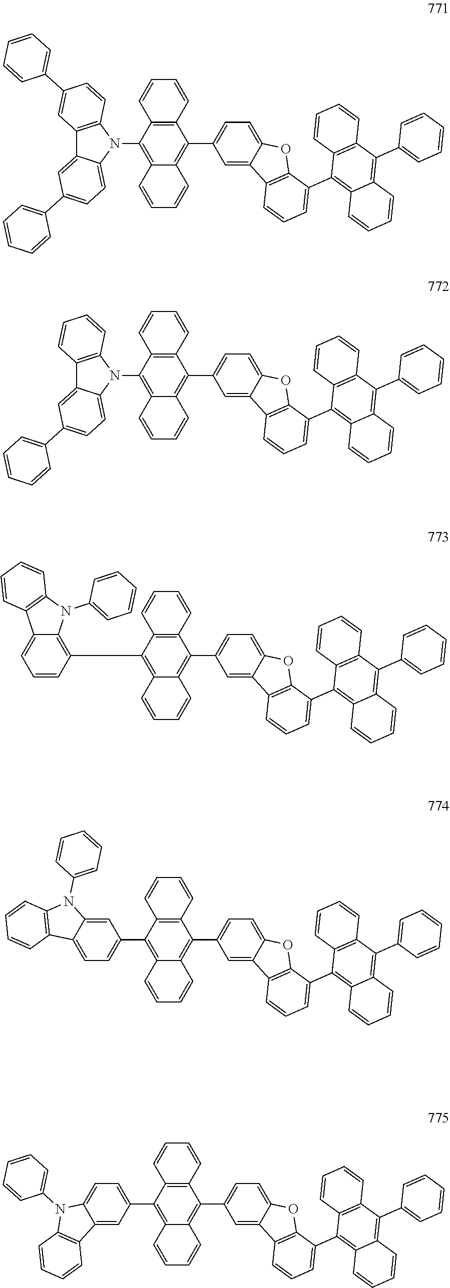

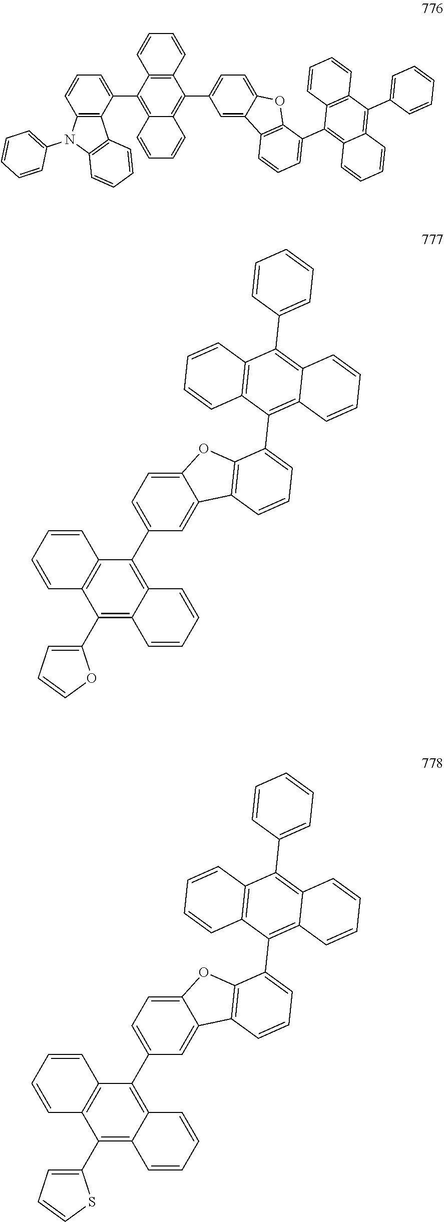 [SMILES: c1ccc(-c2c3ccccc3c(-c3cccc4c3oc3ccc(-c5c6ccccc6c(-c6cccc7c6c6ccccc6n7-c6ccccc6)c6ccccc56)cc34)c3ccccc23)cc1 ; c1ccc(-c2c3ccccc3c(-c3cccc4c3oc3ccc(-c5c6ccccc6c(-c6ccco6)c6ccccc56)cc34)c3ccccc23)cc1 ; c1ccc(-c2c3ccccc3c(-c3cccc4c3oc3ccc(-c5c6ccccc6c(-c6cccs6)c6ccccc56)cc34)c3ccccc23)cc1]

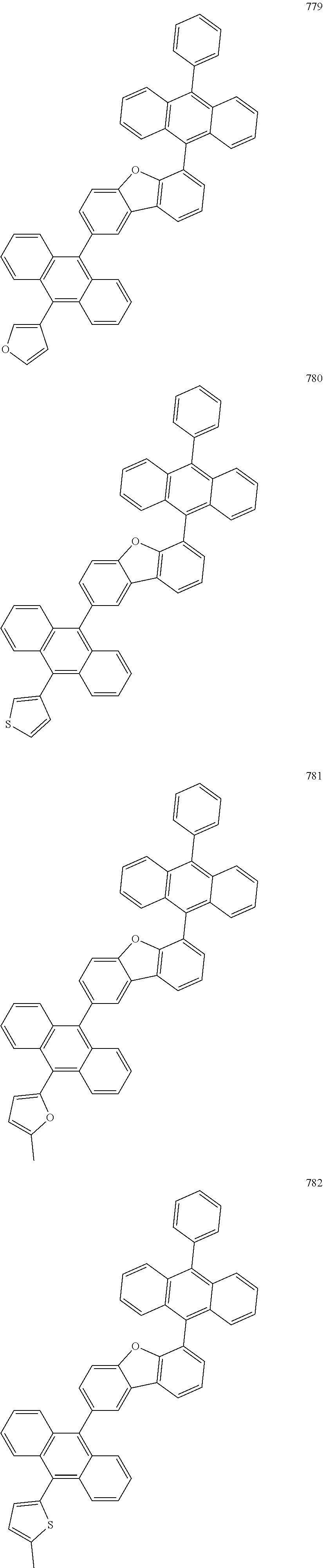 [SMILES: c1ccc(-c2c3ccccc3c(-c3cccc4c3oc3ccc(-c5c6ccccc6c(-c6ccoc6)c6ccccc56)cc34)c3ccccc23)cc1 ; c1ccc(-c2c3ccccc3c(-c3cccc4c3oc3ccc(-c5c6ccccc6c(-c6ccsc6)c6ccccc56)cc34)c3ccccc23)cc1 ; Cc1ccc(-c2c3ccccc3c(-c3ccc4oc5c(-c6c7ccccc7c(-c7ccccc7)c7ccccc67)cccc5c4c3)c3ccccc23)o1 ; Cc1ccc(-c2c3ccccc3c(-c3ccc4oc5c(-c6c7ccccc7c(-c7ccccc7)c7ccccc67)cccc5c4c3)c3ccccc23)s1]

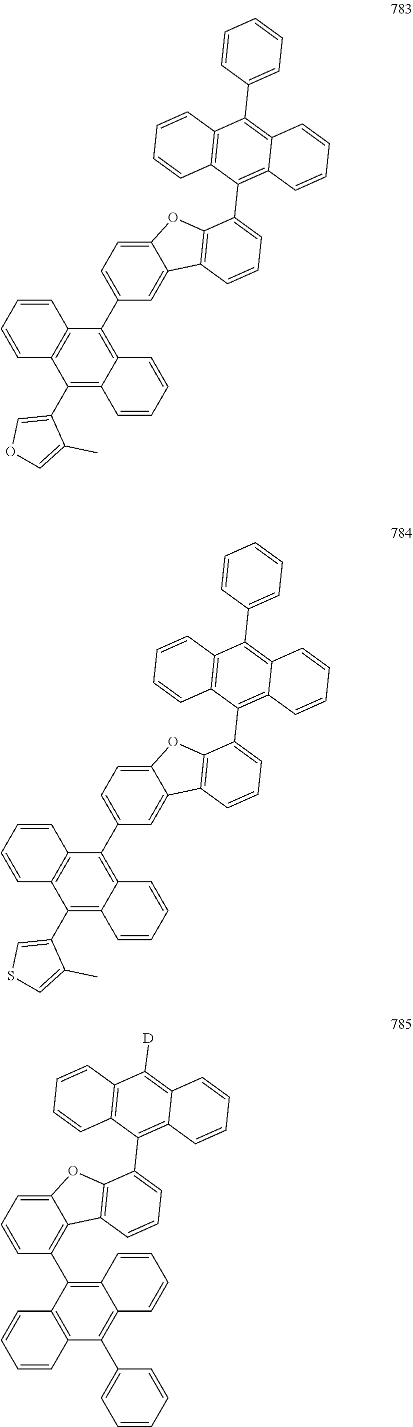 [SMILES: Cc1cocc1-c1c2ccccc2c(-c2ccc3oc4c(-c5c6ccccc6c(-c6ccccc6)c6ccccc56)cccc4c3c2)c2ccccc12 ; Cc1cscc1-c1c2ccccc2c(-c2ccc3oc4c(-c5c6ccccc6c(-c6ccccc6)c6ccccc56)cccc4c3c2)c2ccccc12 ; [2H]c1c2ccccc2c(-c2cccc3c2oc2cccc(-c4c5ccccc5c(-c5ccccc5)c5ccccc45)c23)c2ccccc12]

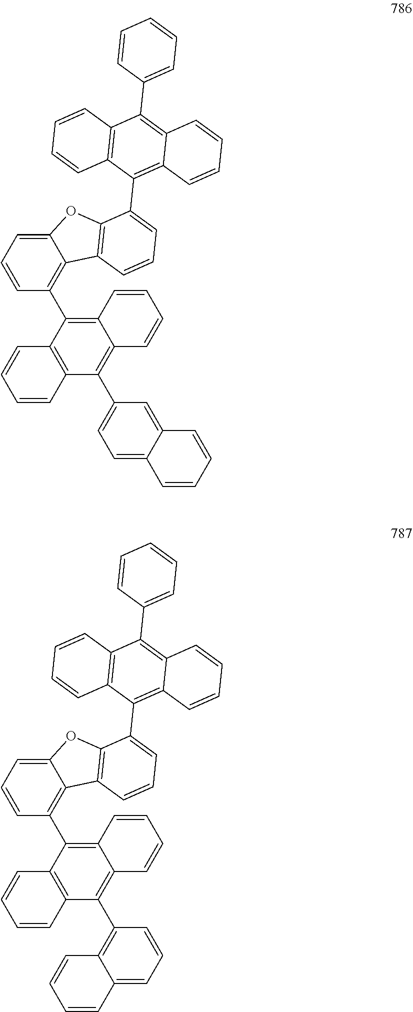 [SMILES: c1ccc(-c2c3ccccc3c(-c3cccc4c3oc3cccc(-c5c6ccccc6c(-c6ccc7ccccc7c6)c6ccccc56)c34)c3ccccc23)cc1 ; c1ccc(-c2c3ccccc3c(-c3cccc4c3oc3cccc(-c5c6ccccc6c(-c6cccc7ccccc67)c6ccccc56)c34)c3ccccc23)cc1]

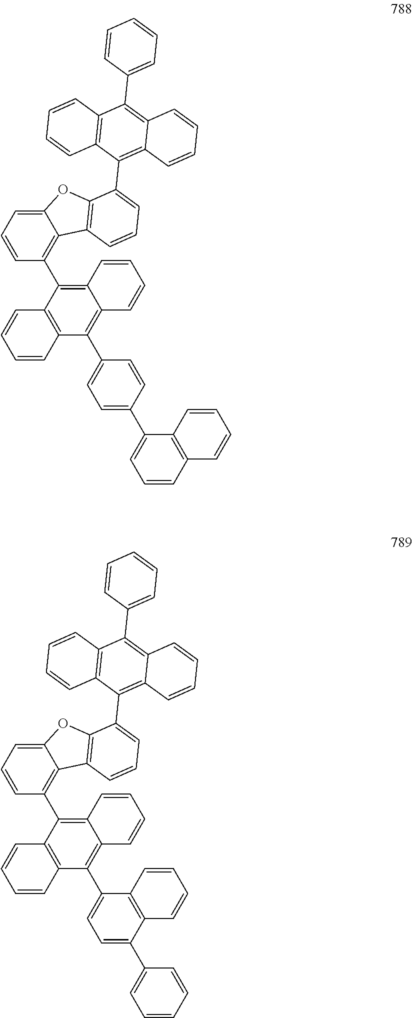 [SMILES: c1ccc(-c2c3ccccc3c(-c3cccc4c3oc3cccc(-c5c6ccccc6c(-c6ccc(-c7cccc8ccccc78)cc6)c6ccccc56)c34)c3ccccc23)cc1 ; c1ccc(-c2ccc(-c3c4ccccc4c(-c4cccc5oc6c(-c7c8ccccc8c(-c8ccccc8)c8ccccc78)cccc6c45)c4ccccc34)c3ccccc23)cc1]

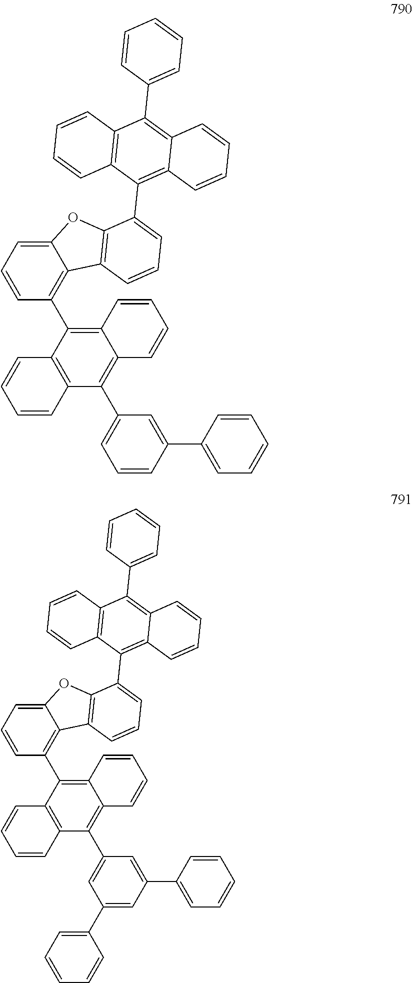

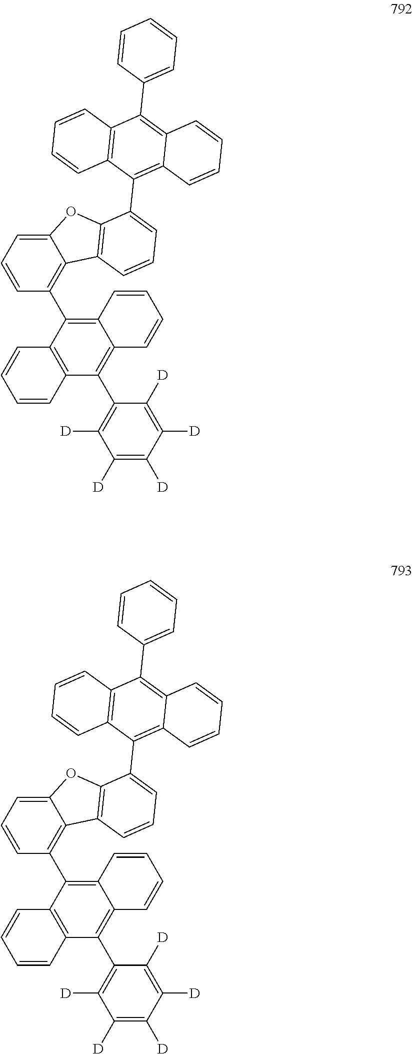 [SMILES: [2H]c1c([2H])c([2H])c(-c2c3ccccc3c(-c3cccc4oc5c(-c6c7ccccc7c(-c7ccccc7)c7ccccc67)cccc5c34)c3ccccc23)c([2H])c1[2H] ; [2H]c1c([2H])c([2H])c(-c2c3ccccc3c(-c3cccc4oc5c(-c6c7ccccc7c(-c7ccccc7)c7ccccc67)cccc5c34)c3ccccc23)c([2H])c1[2H]]

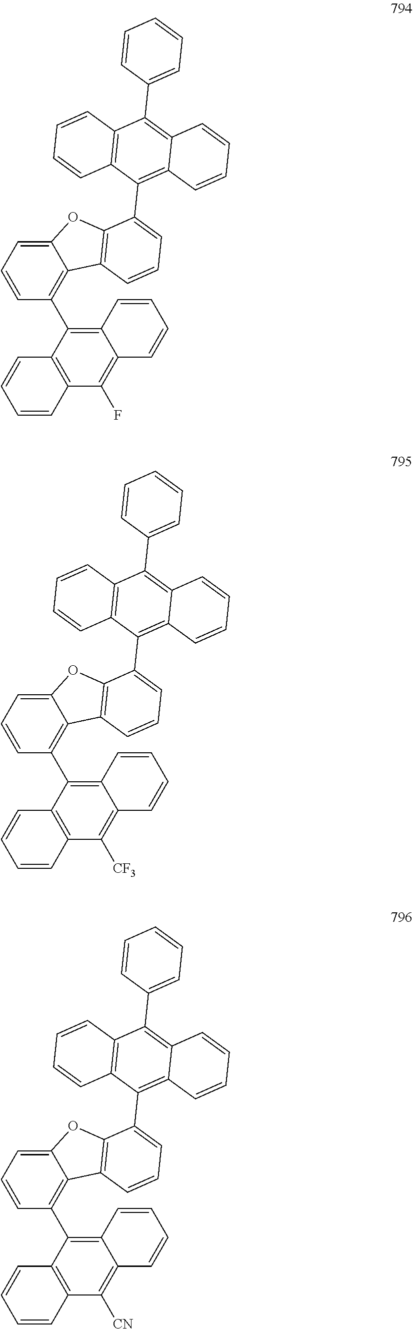 [SMILES: Fc1c2ccccc2c(-c2cccc3oc4c(-c5c6ccccc6c(-c6ccccc6)c6ccccc56)cccc4c23)c2ccccc12 ; FC(F)(F)c1c2ccccc2c(-c2cccc3oc4c(-c5c6ccccc6c(-c6ccccc6)c6ccccc56)cccc4c23)c2ccccc12 ; N#Cc1c2ccccc2c(-c2cccc3oc4c(-c5c6ccccc6c(-c6ccccc6)c6ccccc56)cccc4c23)c2ccccc12]

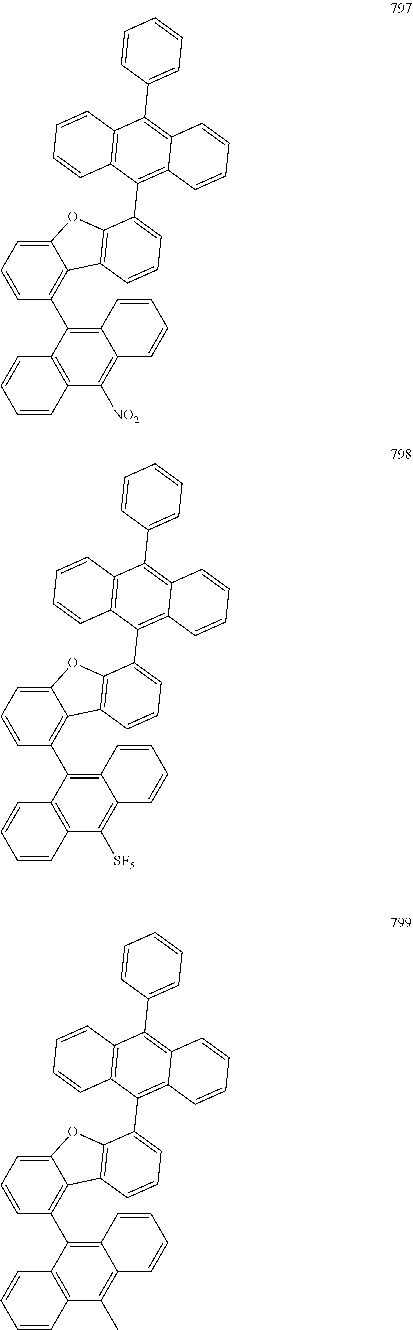

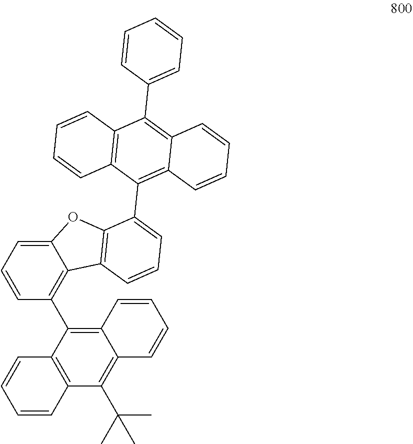 [SMILES: CC(C)(C)c1c2ccccc2c(-c2cccc3oc4c(-c5c6ccccc6c(-c6ccccc6)c6ccccc56)cccc4c23)c2ccccc12]

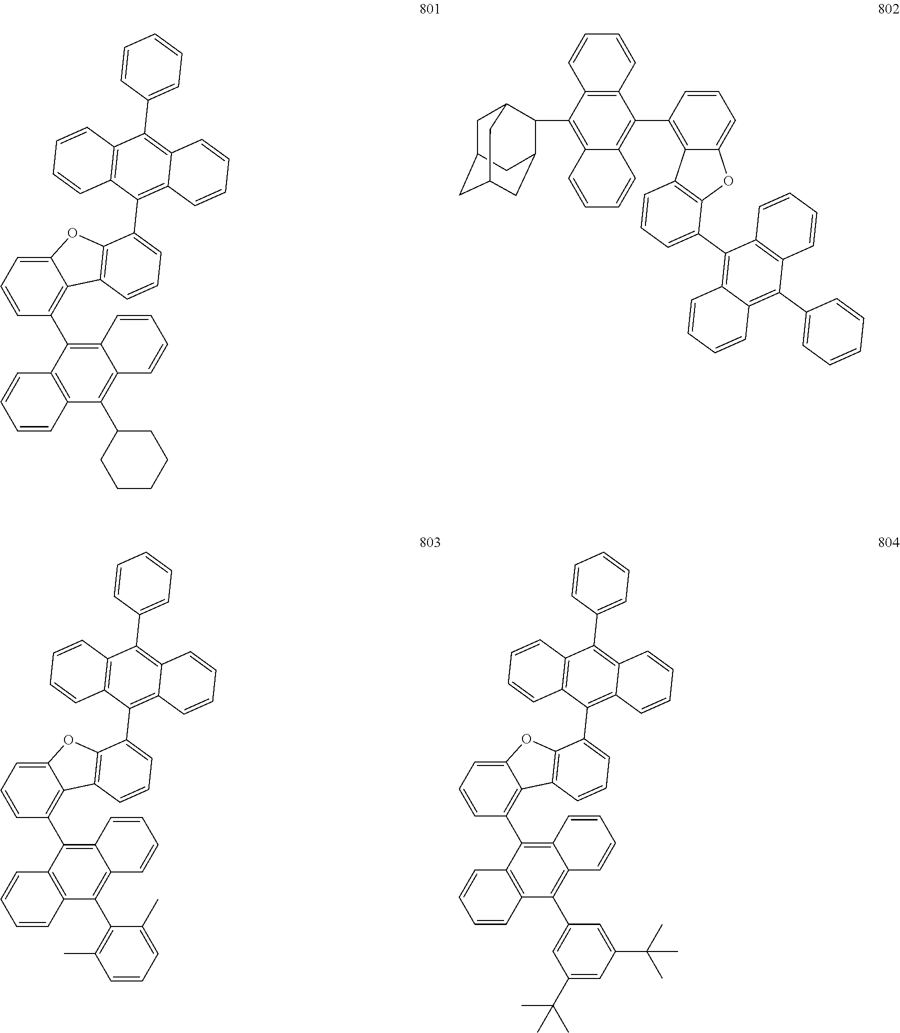 [SMILES: c1ccc(-c2c3ccccc3c(-c3cccc4c3oc3cccc(-c5c6ccccc6c(C6CCCCC6)c6ccccc56)c34)c3ccccc23)cc1 ; c1ccc(-c2c3ccccc3c(-c3cccc4c3oc3cccc(-c5c6ccccc6c(C6C7CC8CC(C7)CC6C8)c6ccccc56)c34)c3ccccc23)cc1 ; Cc1cccc(C)c1-c1c2ccccc2c(-c2cccc3oc4c(-c5c6ccccc6c(-c6ccccc6)c6ccccc56)cccc4c23)c2ccccc12 ; CC(C)(C)c1cc(-c2c3ccccc3c(-c3cccc4oc5c(-c6c7ccccc7c(-c7ccccc7)c7ccccc67)cccc5c34)c3ccccc23)cc(C(C)(C)C)c1]

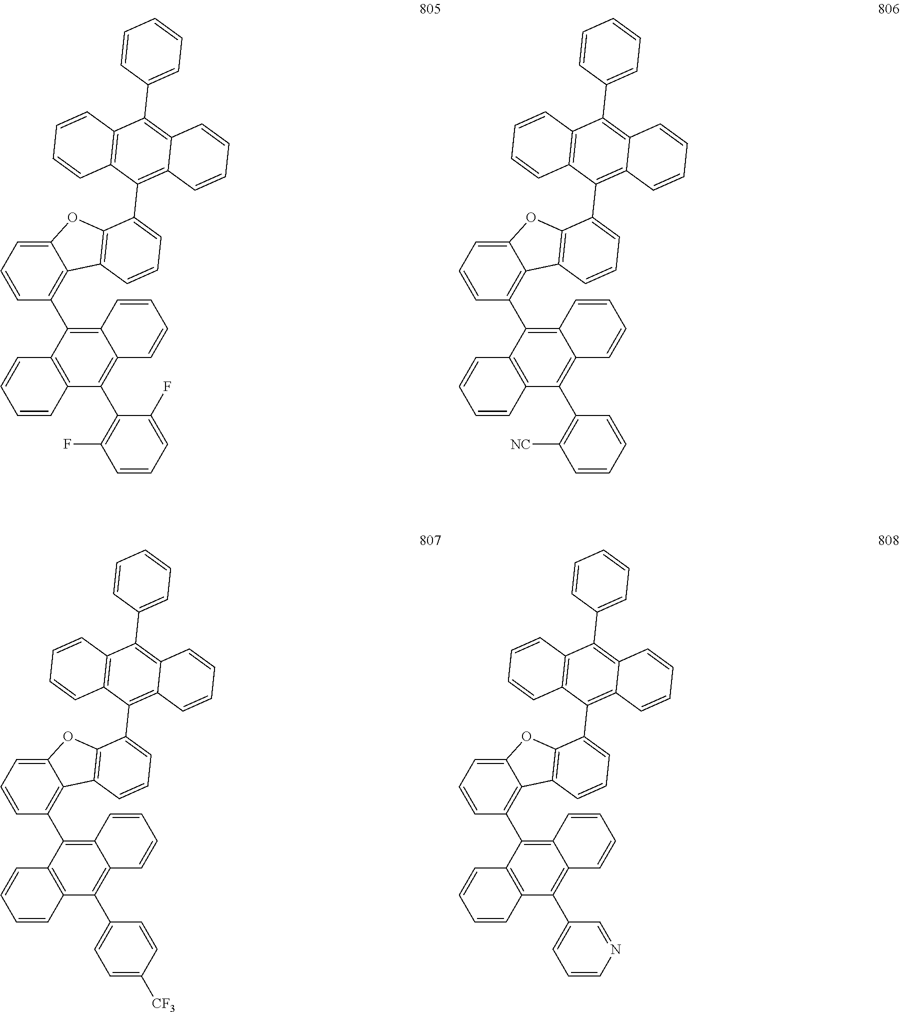 [SMILES: Fc1cccc(F)c1-c1c2ccccc2c(-c2cccc3oc4c(-c5c6ccccc6c(-c6ccccc6)c6ccccc56)cccc4c23)c2ccccc12 ; N#Cc1ccccc1-c1c2ccccc2c(-c2cccc3oc4c(-c5c6ccccc6c(-c6ccccc6)c6ccccc56)cccc4c23)c2ccccc12 ; FC(F)(F)c1ccc(-c2c3ccccc3c(-c3cccc4oc5c(-c6c7ccccc7c(-c7ccccc7)c7ccccc67)cccc5c34)c3ccccc23)cc1 ; c1ccc(-c2c3ccccc3c(-c3cccc4c3oc3cccc(-c5c6ccccc6c(-c6cccnc6)c6ccccc56)c34)c3ccccc23)cc1]

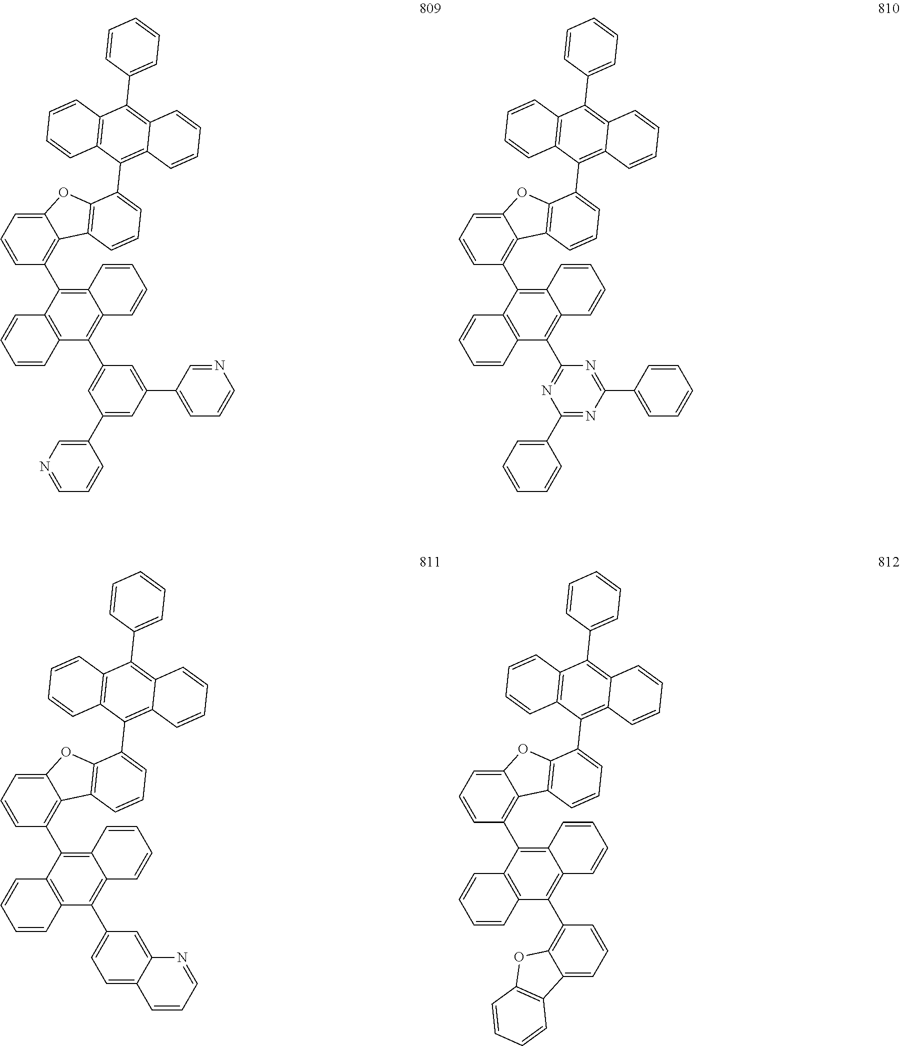 [SMILES: c1ccc(-c2c3ccccc3c(-c3cccc4c3oc3cccc(-c5c6ccccc6c(-c6cc(-c7cccnc7)cc(-c7cccnc7)c6)c6ccccc56)c34)c3ccccc23)cc1 ; c1ccc(-c2nc(-c3ccccc3)nc(-c3c4ccccc4c(-c4cccc5oc6c(-c7c8ccccc8c(-c8ccccc8)c8ccccc78)cccc6c45)c4ccccc34)n2)cc1 ; c1ccc(-c2c3ccccc3c(-c3cccc4c3oc3cccc(-c5c6ccccc6c(-c6ccc7cccnc7c6)c6ccccc56)c34)c3ccccc23)cc1 ; c1ccc(-c2c3ccccc3c(-c3cccc4c3oc3cccc(-c5c6ccccc6c(-c6cccc7c6oc6ccccc67)c6ccccc56)c34)c3ccccc23)cc1]

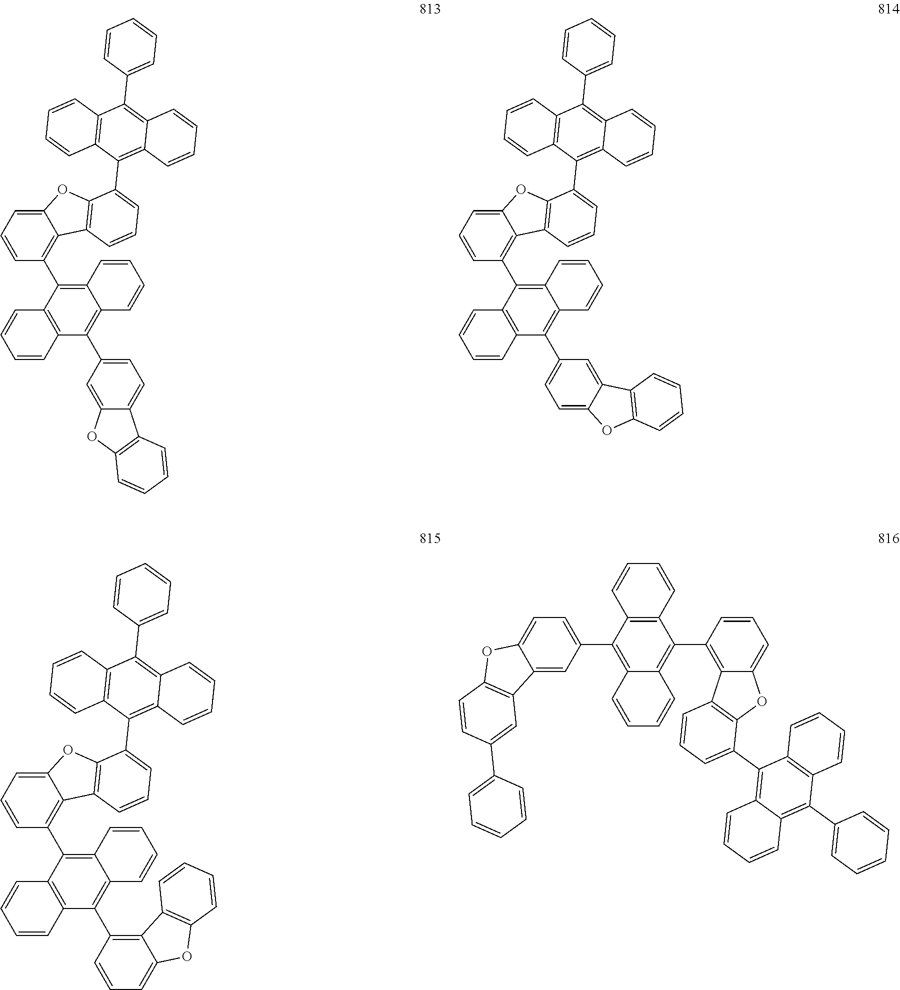 [SMILES: c1ccc(-c2c3ccccc3c(-c3cccc4c3oc3cccc(-c5c6ccccc6c(-c6ccc7c(c6)oc6ccccc67)c6ccccc56)c34)c3ccccc23)cc1 ; c1ccc(-c2c3ccccc3c(-c3cccc4c3oc3cccc(-c5c6ccccc6c(-c6ccc7oc8ccccc8c7c6)c6ccccc56)c34)c3ccccc23)cc1 ; c1ccc(-c2c3ccccc3c(-c3cccc4c3oc3cccc(-c5c6ccccc6c(-c6cccc7oc8ccccc8c67)c6ccccc56)c34)c3ccccc23)cc1 ; c1ccc(-c2ccc3oc4ccc(-c5c6ccccc6c(-c6cccc7oc8c(-c9c%10ccccc%10c(-c%10ccccc%10)c%10ccccc9%10)cccc8c67)c6ccccc56)cc4c3c2)cc1]

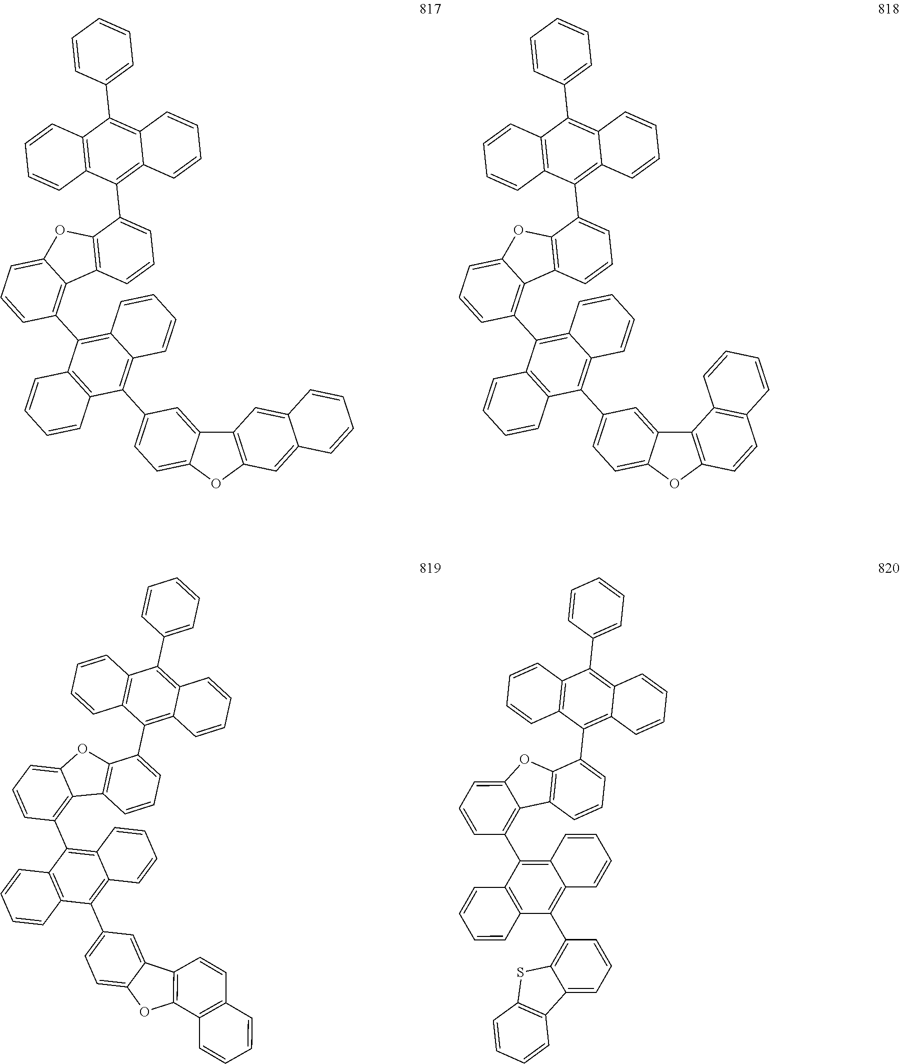 [SMILES: c1ccc(-c2c3ccccc3c(-c3cccc4c3oc3cccc(-c5c6ccccc6c(-c6ccc7oc8cc9ccccc9cc8c7c6)c6ccccc56)c34)c3ccccc23)cc1 ; c1ccc(-c2c3ccccc3c(-c3cccc4c3oc3cccc(-c5c6ccccc6c(-c6ccc7oc8ccc9ccccc9c8c7c6)c6ccccc56)c34)c3ccccc23)cc1 ; c1ccc(-c2c3ccccc3c(-c3cccc4c3oc3cccc(-c5c6ccccc6c(-c6ccc7oc8c9ccccc9ccc8c7c6)c6ccccc56)c34)c3ccccc23)cc1 ; c1ccc(-c2c3ccccc3c(-c3cccc4c3oc3cccc(-c5c6ccccc6c(-c6cccc7c6sc6ccccc67)c6ccccc56)c34)c3ccccc23)cc1]

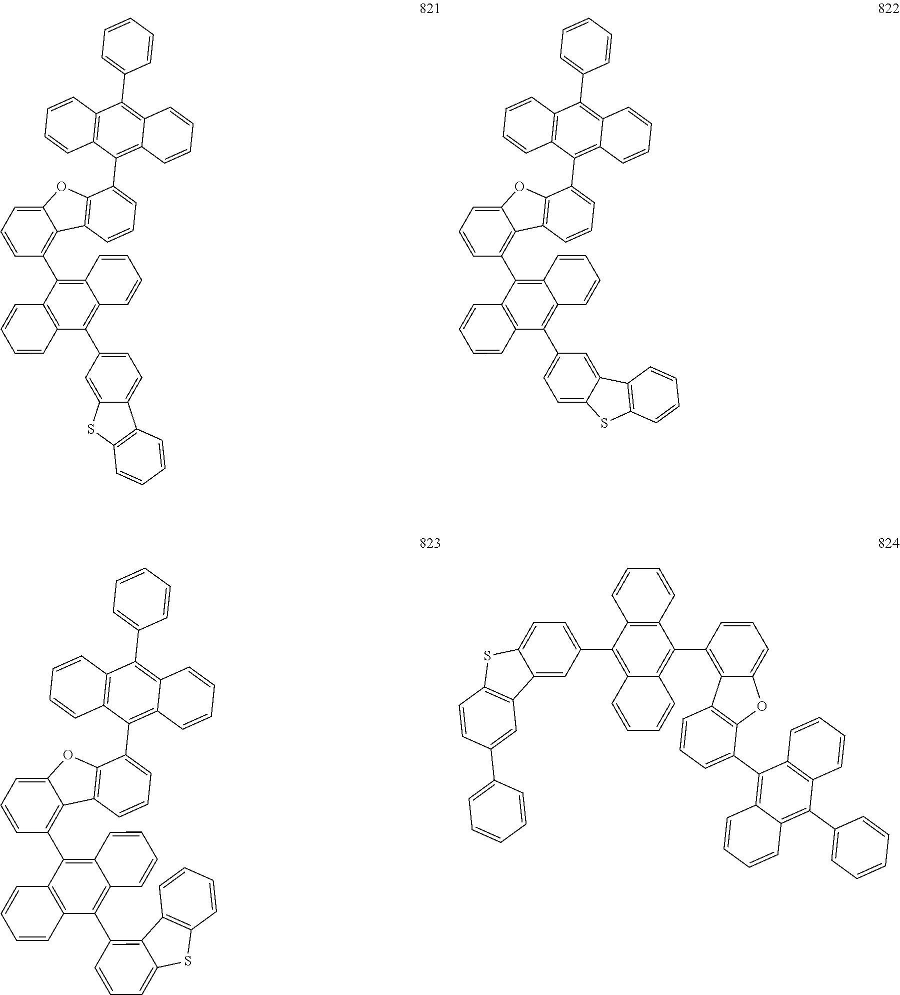 [SMILES: c1ccc(-c2c3ccccc3c(-c3cccc4c3oc3cccc(-c5c6ccccc6c(-c6ccc7c(c6)sc6ccccc67)c6ccccc56)c34)c3ccccc23)cc1 ; c1ccc(-c2c3ccccc3c(-c3cccc4c3oc3cccc(-c5c6ccccc6c(-c6ccc7sc8ccccc8c7c6)c6ccccc56)c34)c3ccccc23)cc1 ; c1ccc(-c2c3ccccc3c(-c3cccc4c3oc3cccc(-c5c6ccccc6c(-c6cccc7sc8ccccc8c67)c6ccccc56)c34)c3ccccc23)cc1 ; c1ccc(-c2ccc3sc4ccc(-c5c6ccccc6c(-c6cccc7oc8c(-c9c%10ccccc%10c(-c%10ccccc%10)c%10ccccc9%10)cccc8c67)c6ccccc56)cc4c3c2)cc1]

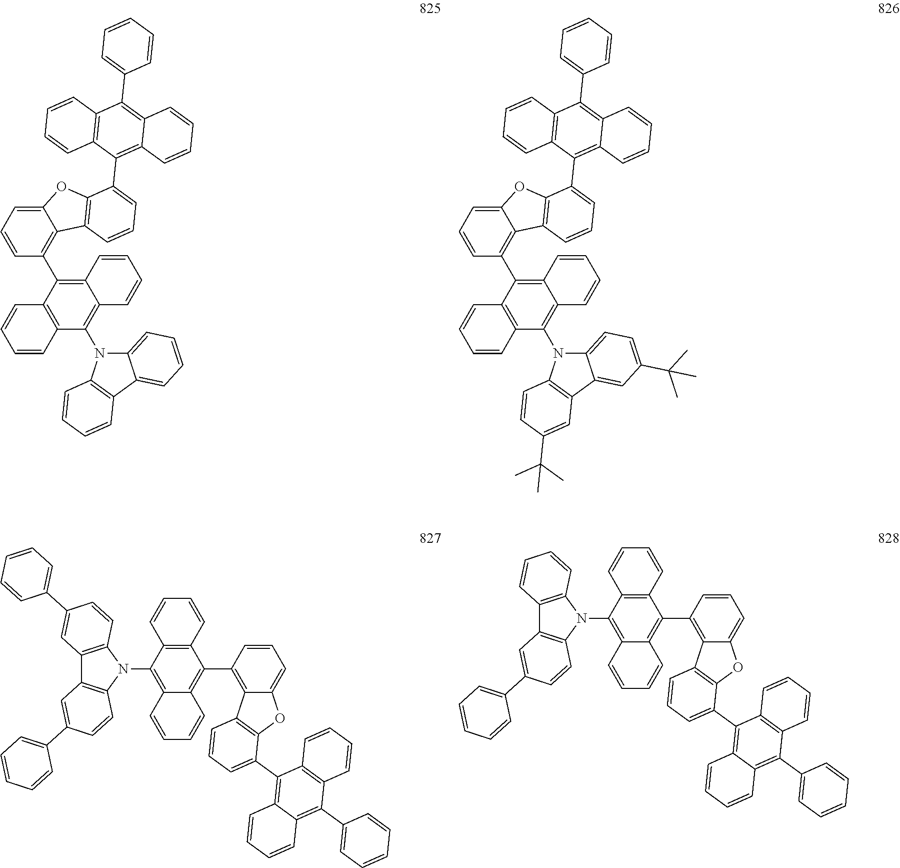 [SMILES: c1ccc(-c2c3ccccc3c(-c3cccc4c3oc3cccc(-c5c6ccccc6c(-n6c7ccccc7c7ccccc76)c6ccccc56)c34)c3ccccc23)cc1 ; CC(C)(C)c1ccc2c(c1)c1cc(C(C)(C)C)ccc1n2-c1c2ccccc2c(-c2cccc3oc4c(-c5c6ccccc6c(-c6ccccc6)c6ccccc56)cccc4c23)c2ccccc12 ; c1ccc(-c2ccc3c(c2)c2cc(-c4ccccc4)ccc2n3-c2c3ccccc3c(-c3cccc4oc5c(-c6c7ccccc7c(-c7ccccc7)c7ccccc67)cccc5c34)c3ccccc23)cc1 ; c1ccc(-c2ccc3c(c2)c2ccccc2n3-c2c3ccccc3c(-c3cccc4oc5c(-c6c7ccccc7c(-c7ccccc7)c7ccccc67)cccc5c34)c3ccccc23)cc1]

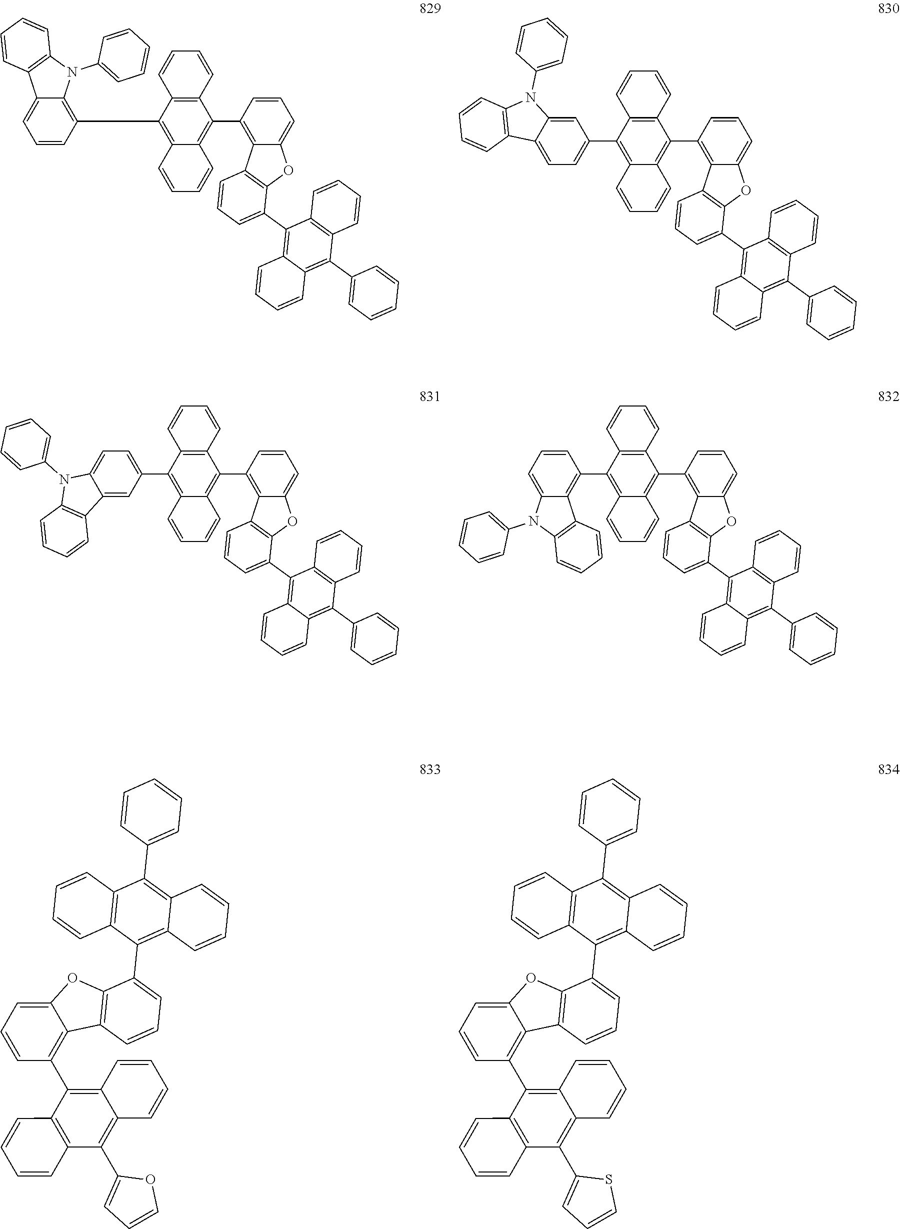

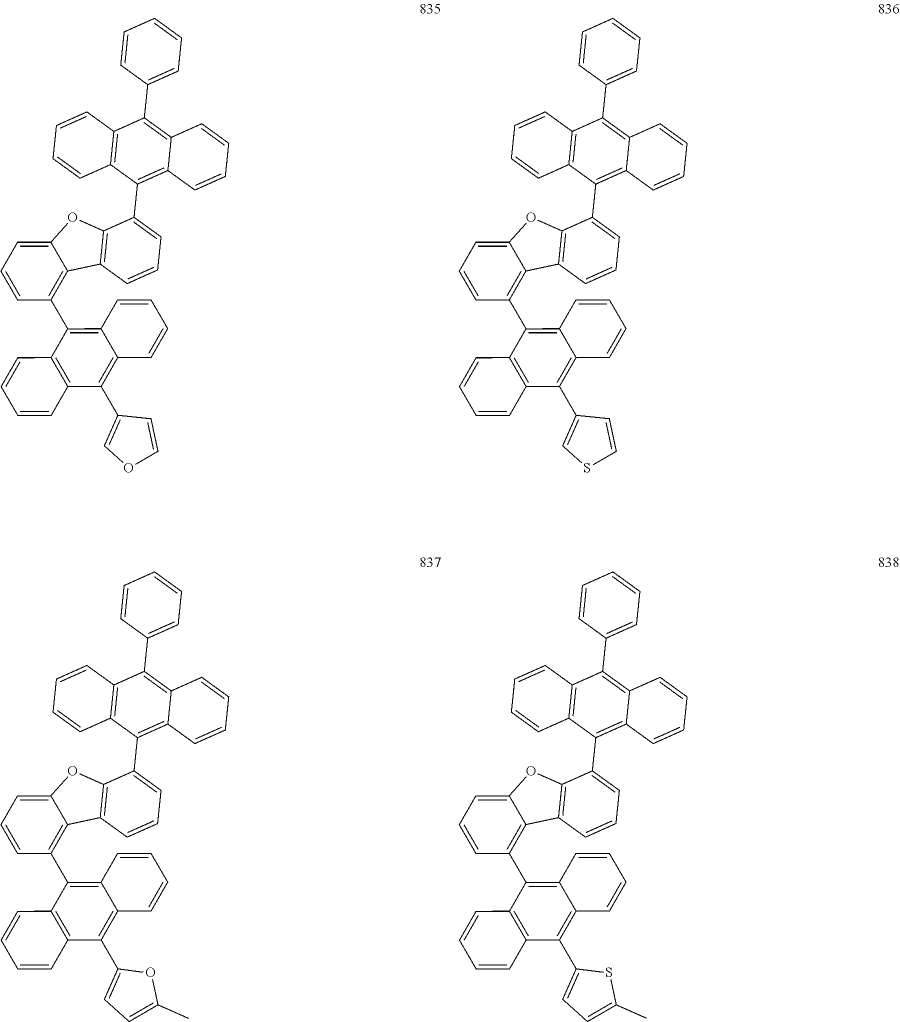 [SMILES: c1ccc(-c2c3ccccc3c(-c3cccc4c3oc3cccc(-c5c6ccccc6c(-c6ccoc6)c6ccccc56)c34)c3ccccc23)cc1 ; c1ccc(-c2c3ccccc3c(-c3cccc4c3oc3cccc(-c5c6ccccc6c(-c6ccsc6)c6ccccc56)c34)c3ccccc23)cc1 ; Cc1ccc(-c2c3ccccc3c(-c3cccc4oc5c(-c6c7ccccc7c(-c7ccccc7)c7ccccc67)cccc5c34)c3ccccc23)o1 ; Cc1ccc(-c2c3ccccc3c(-c3cccc4oc5c(-c6c7ccccc7c(-c7ccccc7)c7ccccc67)cccc5c34)c3ccccc23)s1]

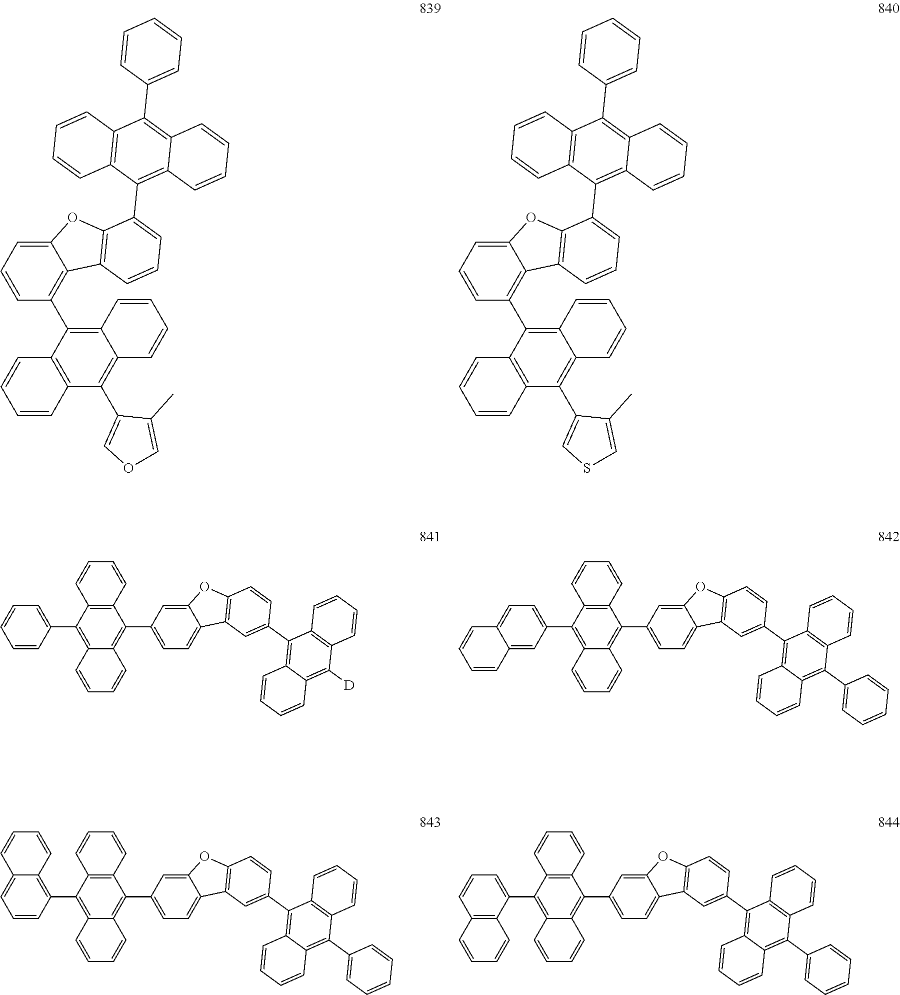

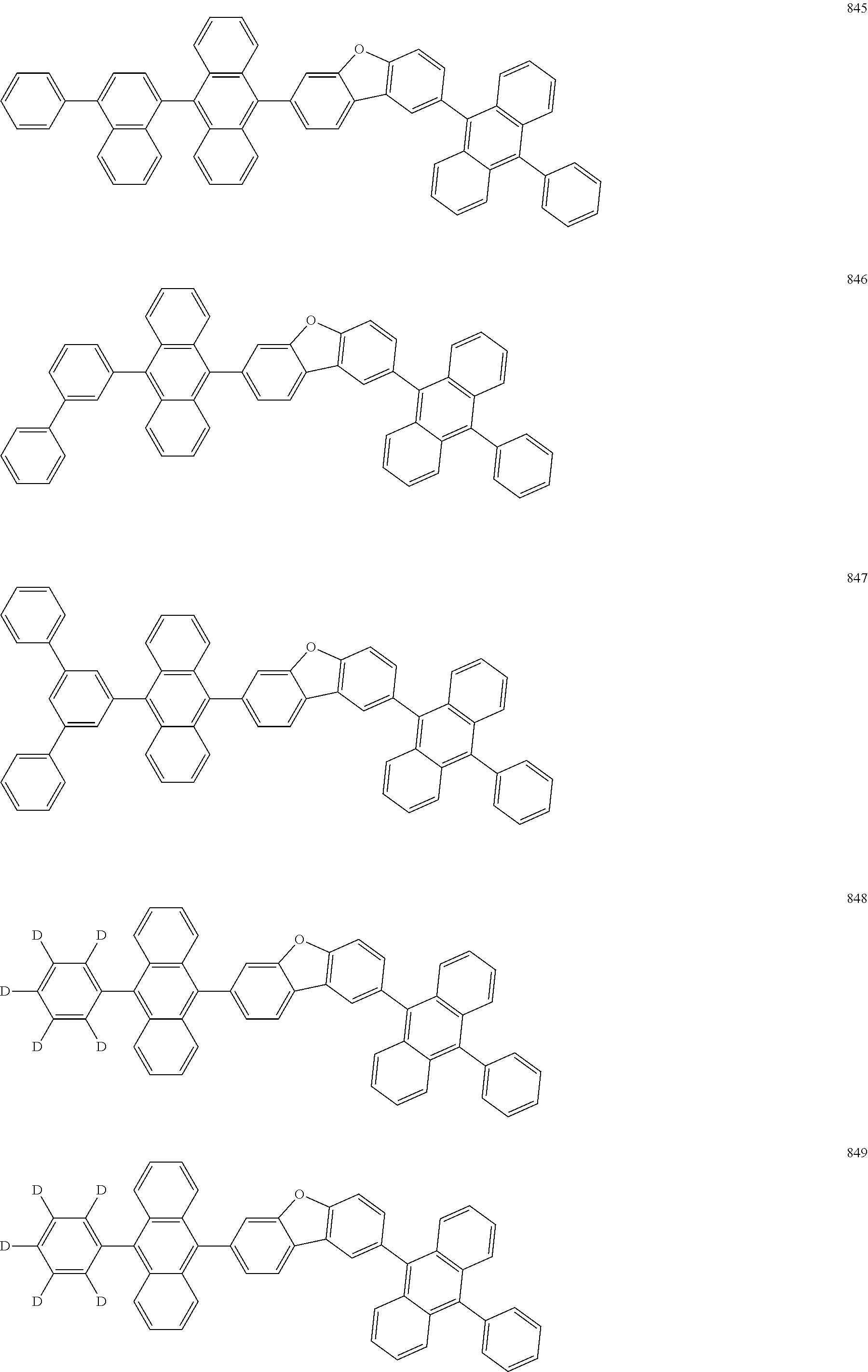

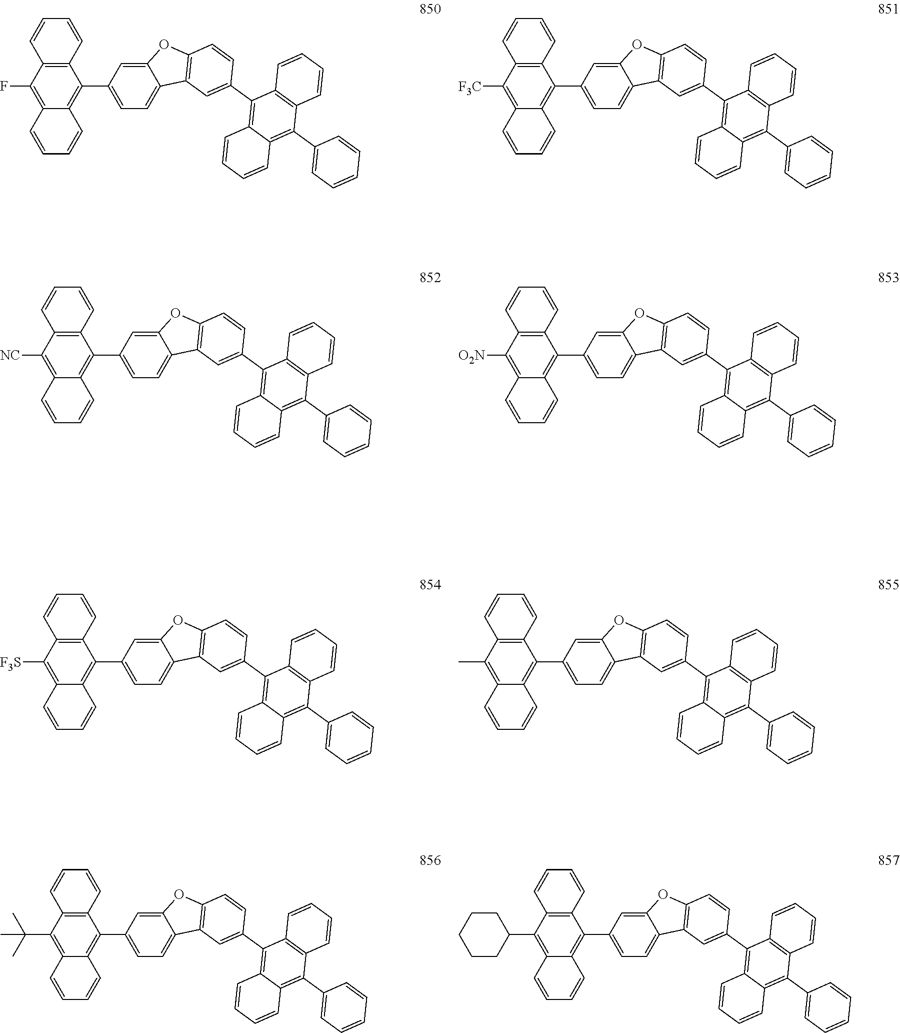

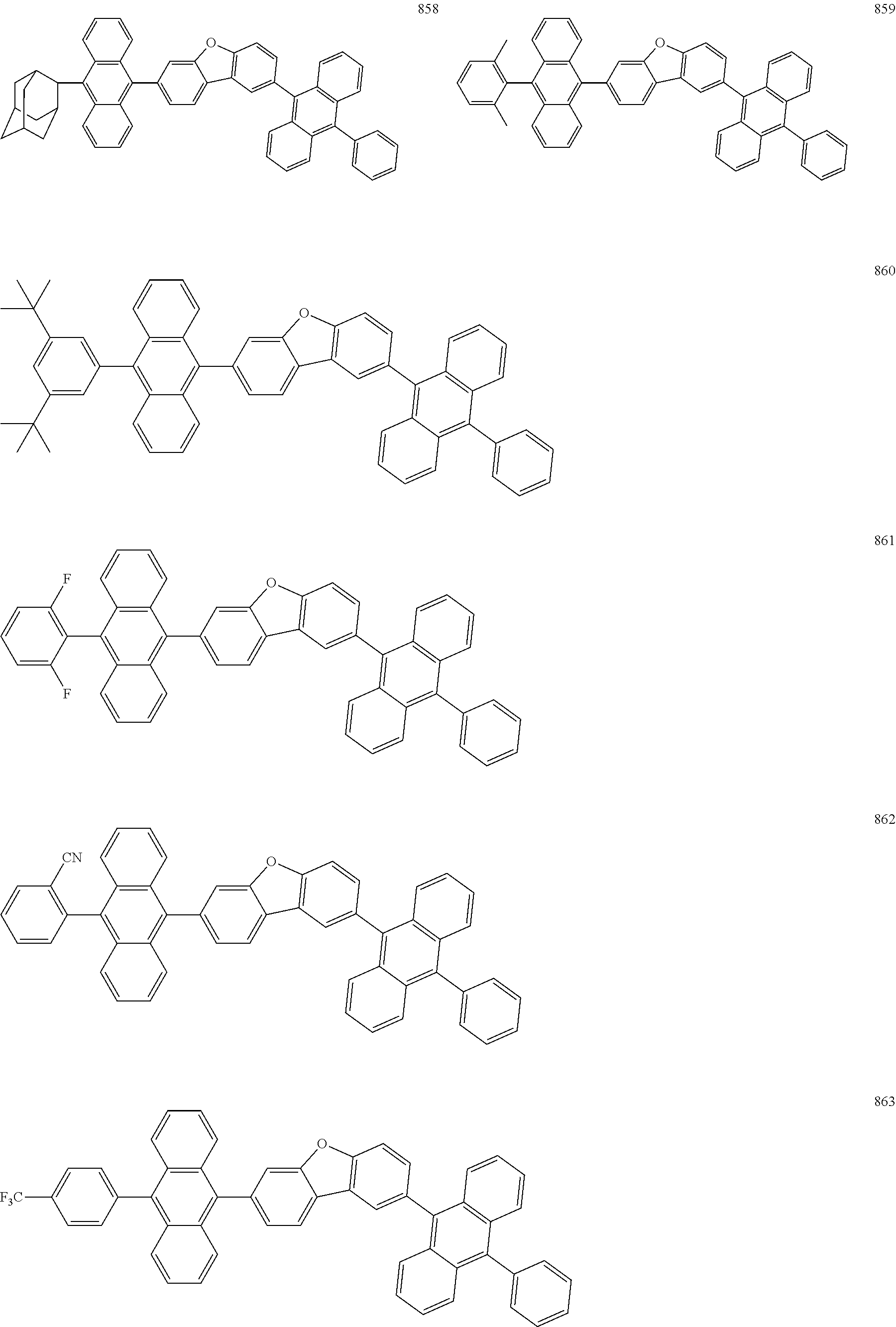

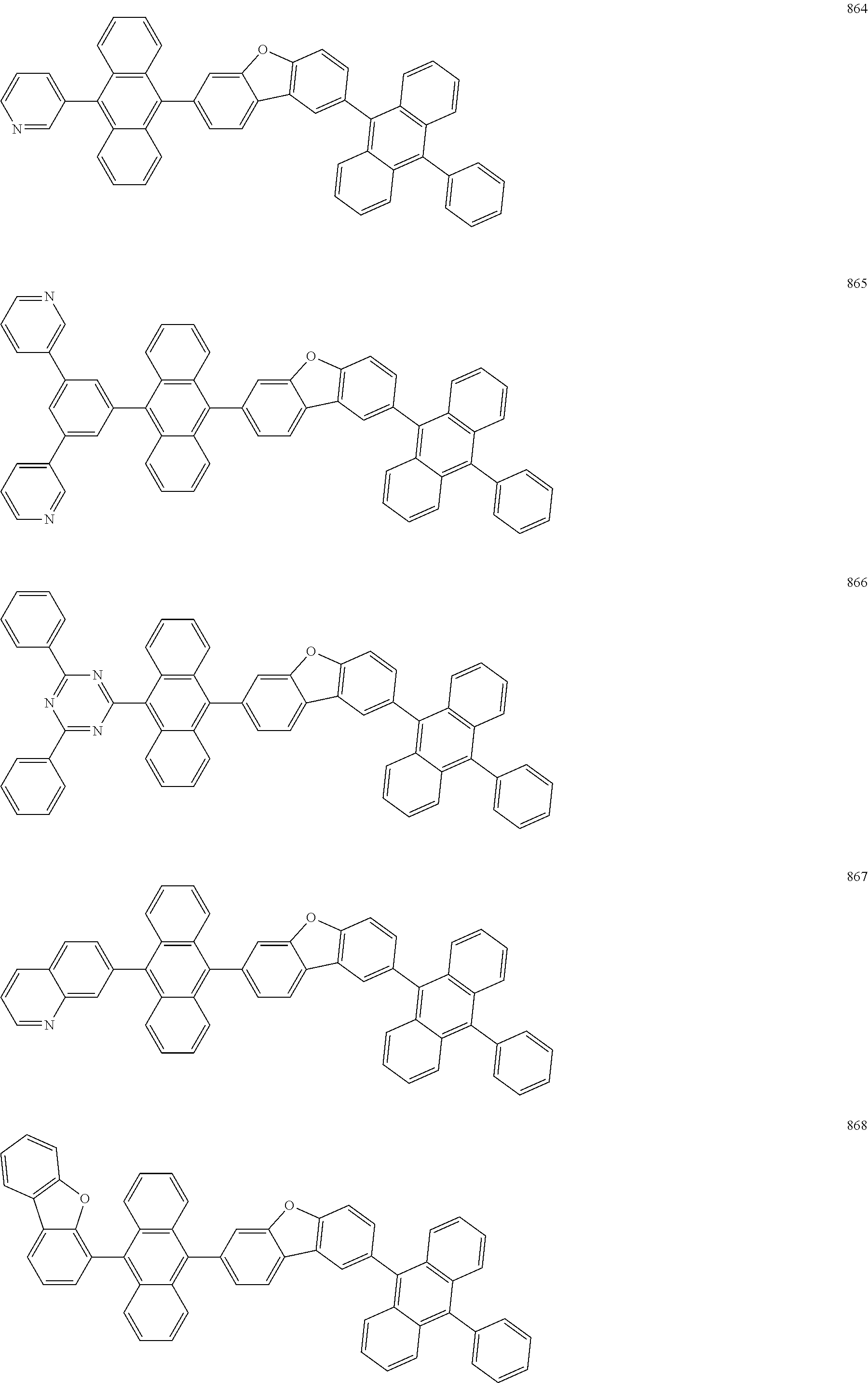

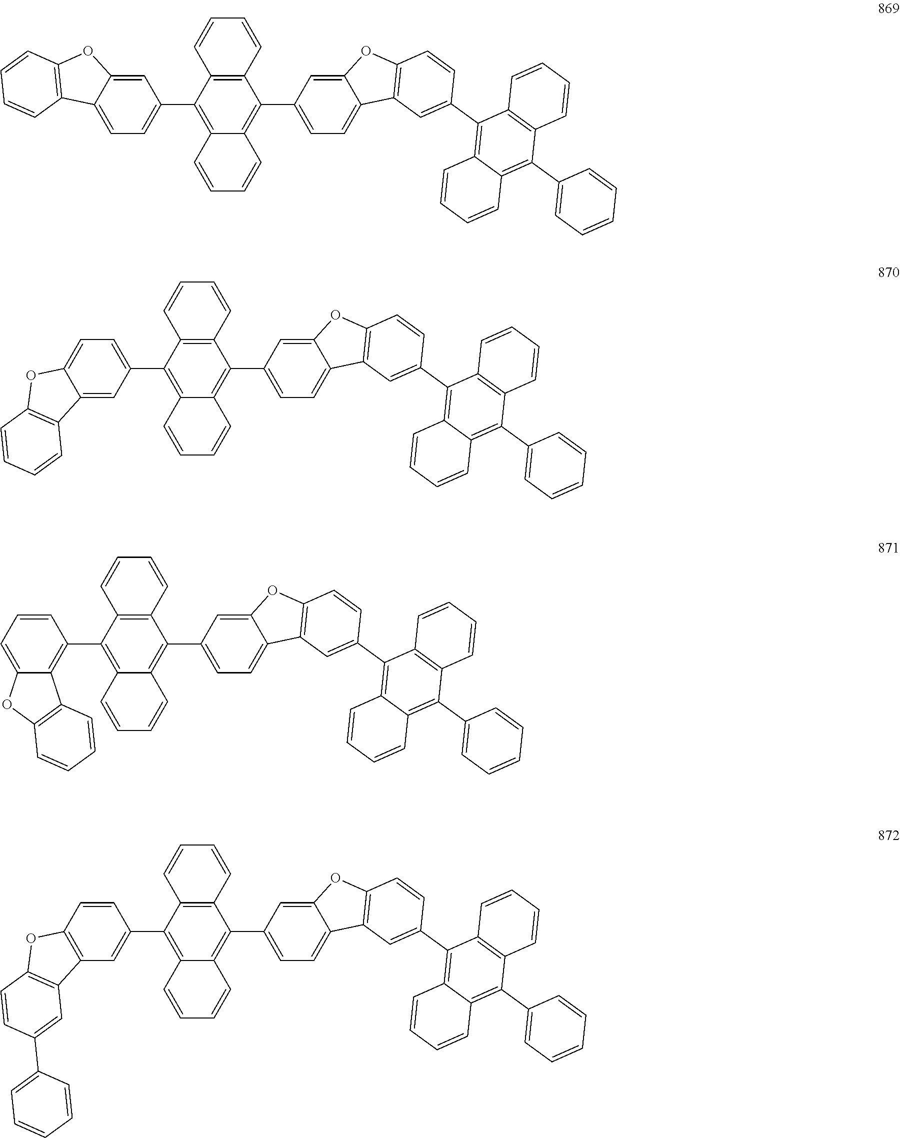 [SMILES: c1ccc(-c2c3ccccc3c(-c3ccc4oc5cc(-c6c7ccccc7c(-c7ccc8c(c7)oc7ccccc78)c7ccccc67)ccc5c4c3)c3ccccc23)cc1 ; c1ccc(-c2c3ccccc3c(-c3ccc4oc5cc(-c6c7ccccc7c(-c7ccc8oc9ccccc9c8c7)c7ccccc67)ccc5c4c3)c3ccccc23)cc1 ; c1ccc(-c2c3ccccc3c(-c3ccc4oc5cc(-c6c7ccccc7c(-c7cccc8oc9ccccc9c78)c7ccccc67)ccc5c4c3)c3ccccc23)cc1 ; c1ccc(-c2ccc3oc4ccc(-c5c6ccccc6c(-c6ccc7c(c6)oc6ccc(-c8c9ccccc9c(-c9ccccc9)c9ccccc89)cc67)c6ccccc56)cc4c3c2)cc1]

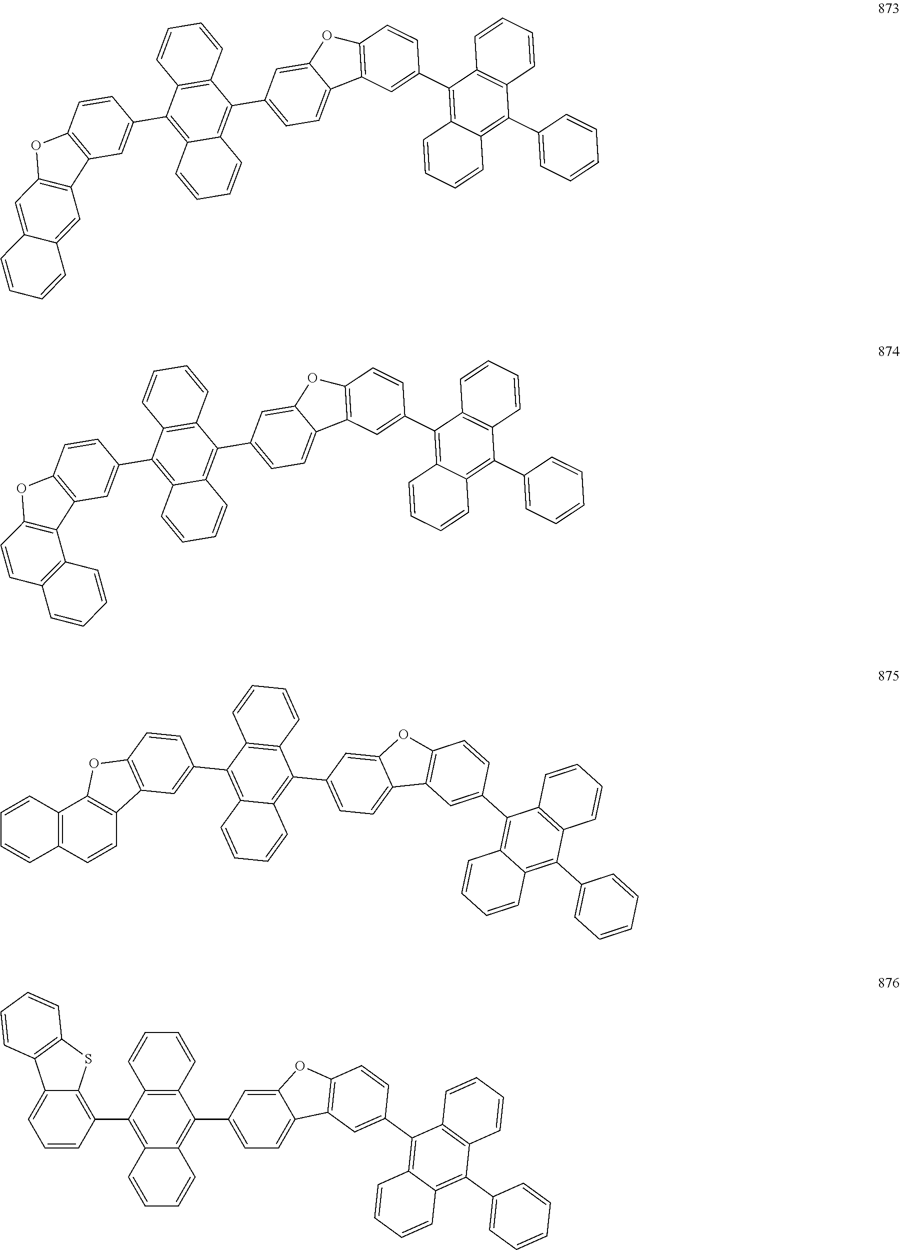 [SMILES: c1ccc(-c2c3ccccc3c(-c3ccc4oc5cc(-c6c7ccccc7c(-c7ccc8oc9cc%10ccccc%10cc9c8c7)c7ccccc67)ccc5c4c3)c3ccccc23)cc1 ; c1ccc(-c2c3ccccc3c(-c3ccc4oc5cc(-c6c7ccccc7c(-c7ccc8oc9ccc%10ccccc%10c9c8c7)c7ccccc67)ccc5c4c3)c3ccccc23)cc1 ; c1ccc(-c2c3ccccc3c(-c3ccc4oc5cc(-c6c7ccccc7c(-c7ccc8oc9c%10ccccc%10ccc9c8c7)c7ccccc67)ccc5c4c3)c3ccccc23)cc1 ; c1ccc(-c2c3ccccc3c(-c3ccc4oc5cc(-c6c7ccccc7c(-c7cccc8c7sc7ccccc78)c7ccccc67)ccc5c4c3)c3ccccc23)cc1]

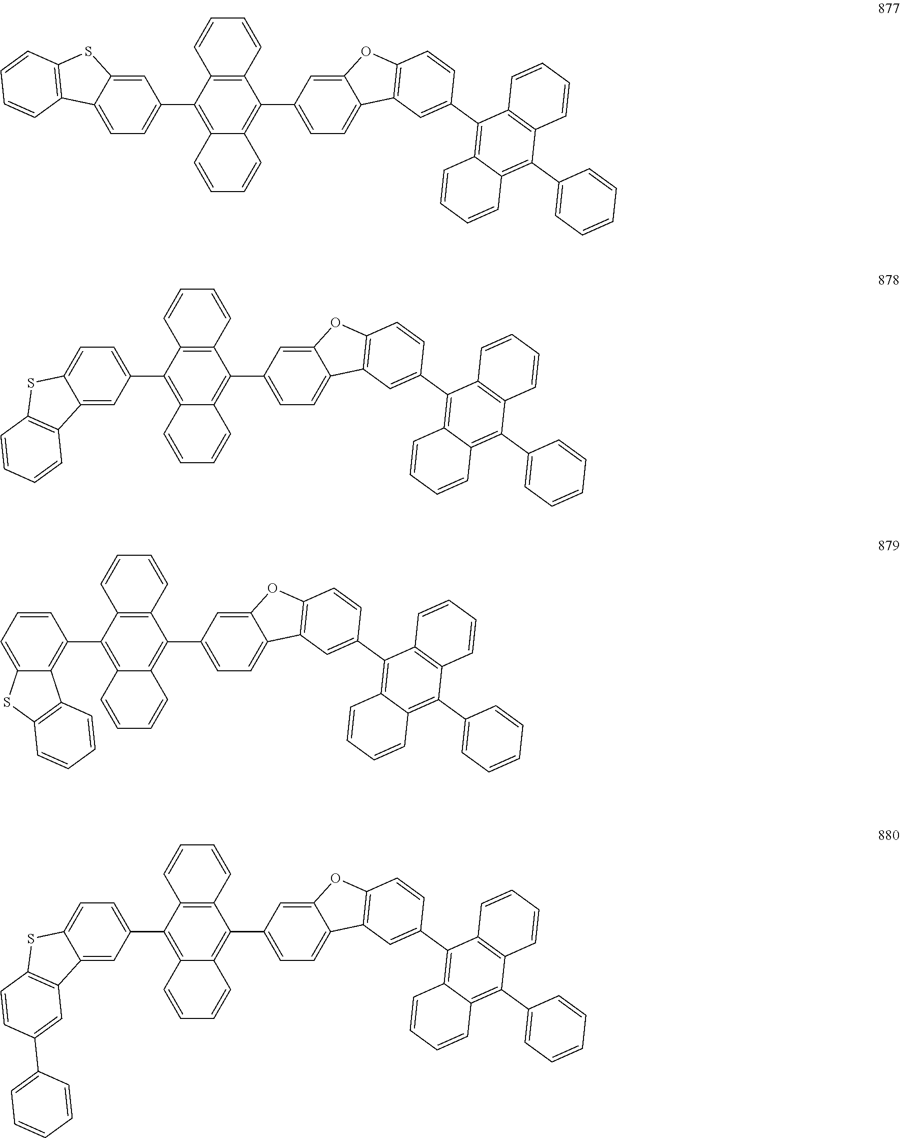 [SMILES: c1ccc(-c2c3ccccc3c(-c3ccc4oc5cc(-c6c7ccccc7c(-c7ccc8c(c7)sc7ccccc78)c7ccccc67)ccc5c4c3)c3ccccc23)cc1 ; c1ccc(-c2c3ccccc3c(-c3ccc4oc5cc(-c6c7ccccc7c(-c7ccc8sc9ccccc9c8c7)c7ccccc67)ccc5c4c3)c3ccccc23)cc1 ; c1ccc(-c2c3ccccc3c(-c3ccc4oc5cc(-c6c7ccccc7c(-c7cccc8sc9ccccc9c78)c7ccccc67)ccc5c4c3)c3ccccc23)cc1 ; c1ccc(-c2ccc3sc4ccc(-c5c6ccccc6c(-c6ccc7c(c6)oc6ccc(-c8c9ccccc9c(-c9ccccc9)c9ccccc89)cc67)c6ccccc56)cc4c3c2)cc1]

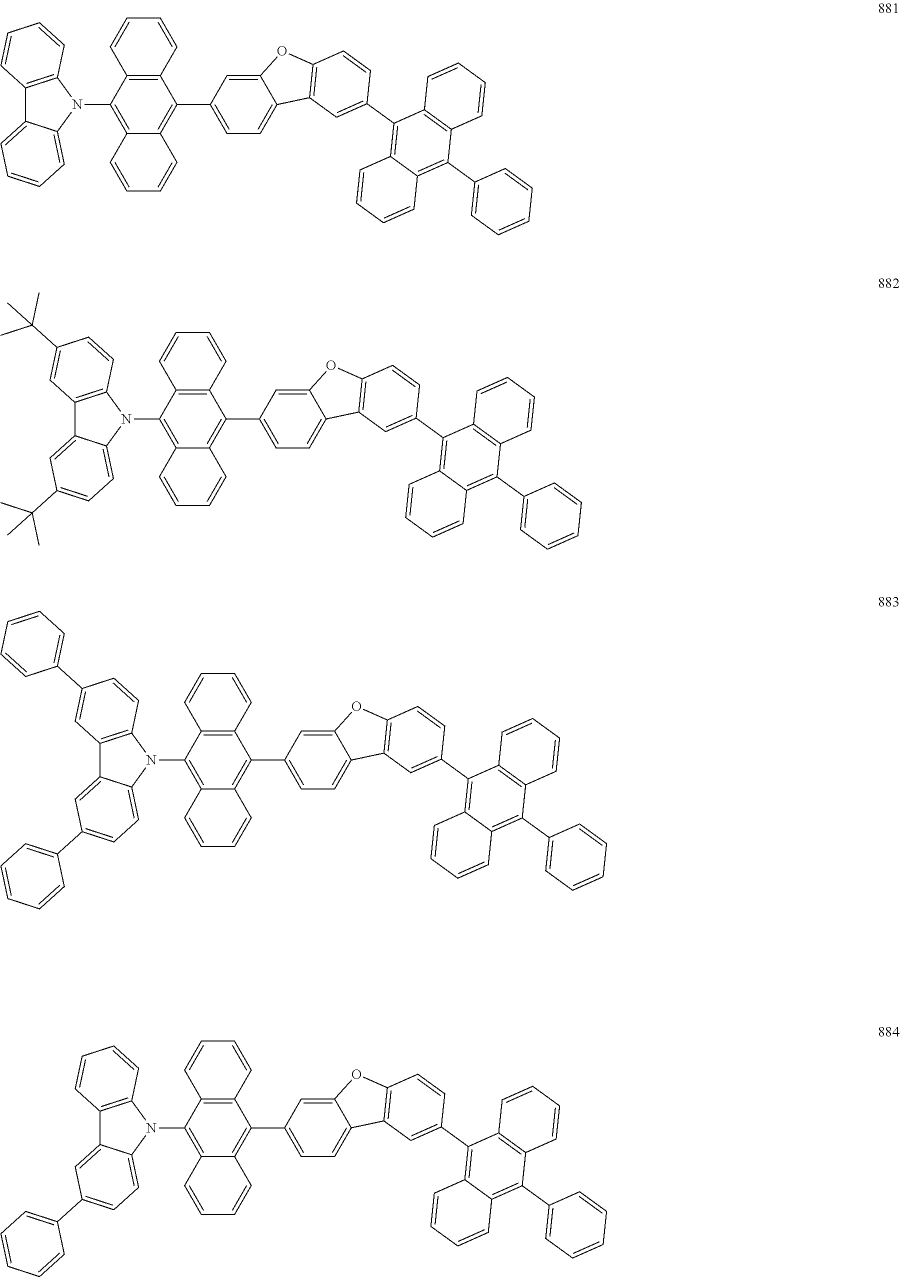 [SMILES: c1ccc(-c2c3ccccc3c(-c3ccc4oc5cc(-c6c7ccccc7c(-n7c8ccccc8c8ccccc87)c7ccccc67)ccc5c4c3)c3ccccc23)cc1 ; CC(C)(C)c1ccc2c(c1)c1cc(C(C)(C)C)ccc1n2-c1c2ccccc2c(-c2ccc3c(c2)oc2ccc(-c4c5ccccc5c(-c5ccccc5)c5ccccc45)cc23)c2ccccc12 ; c1ccc(-c2ccc3c(c2)c2cc(-c4ccccc4)ccc2n3-c2c3ccccc3c(-c3ccc4c(c3)oc3ccc(-c5c6ccccc6c(-c6ccccc6)c6ccccc56)cc34)c3ccccc23)cc1 ; c1ccc(-c2ccc3c(c2)c2ccccc2n3-c2c3ccccc3c(-c3ccc4c(c3)oc3ccc(-c5c6ccccc6c(-c6ccccc6)c6ccccc56)cc34)c3ccccc23)cc1]

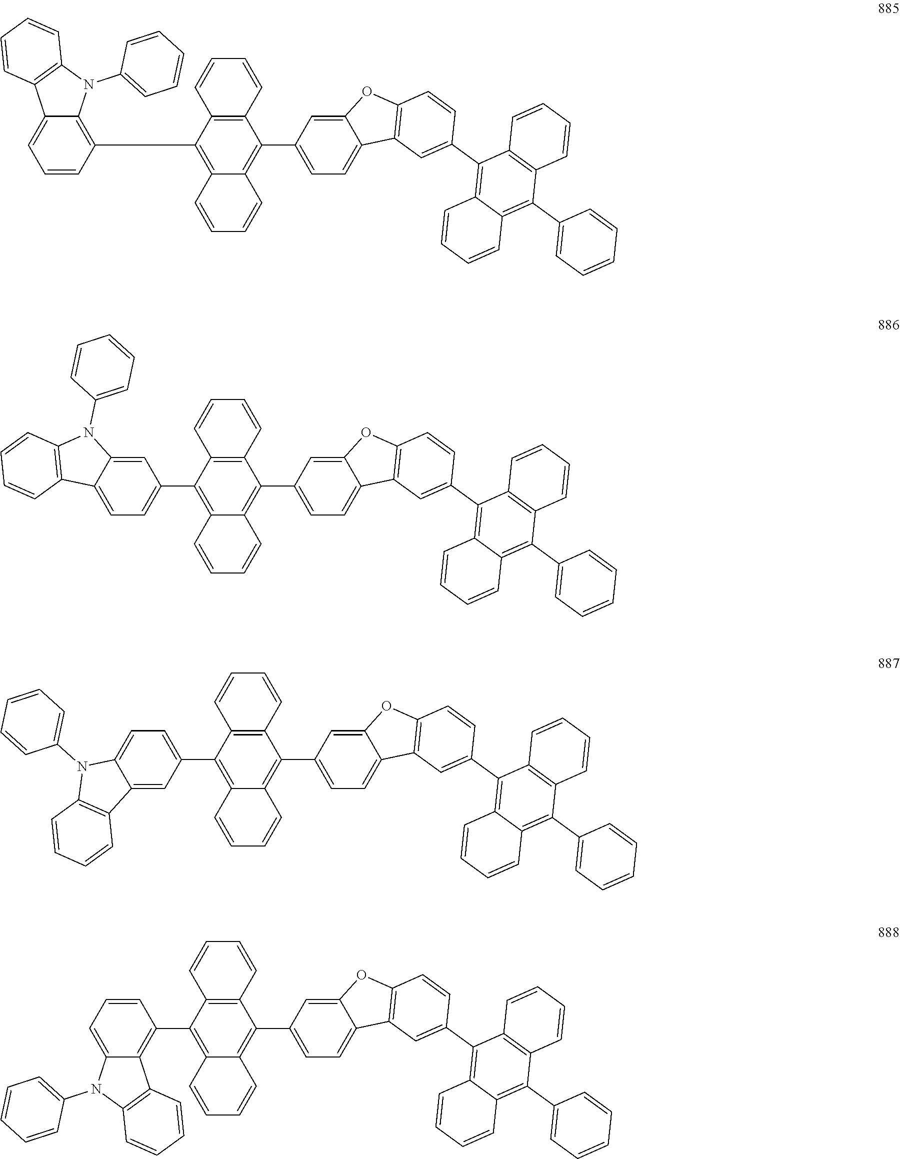 [SMILES: c1ccc(-c2c3ccccc3c(-c3ccc4oc5cc(-c6c7ccccc7c(-c7cccc8c9ccccc9n(-c9ccccc9)c78)c7ccccc67)ccc5c4c3)c3ccccc23)cc1 ; c1ccc(-c2c3ccccc3c(-c3ccc4oc5cc(-c6c7ccccc7c(-c7ccc8c9ccccc9n(-c9ccccc9)c8c7)c7ccccc67)ccc5c4c3)c3ccccc23)cc1 ; c1ccc(-c2c3ccccc3c(-c3ccc4oc5cc(-c6c7ccccc7c(-c7ccc8c(c7)c7ccccc7n8-c7ccccc7)c7ccccc67)ccc5c4c3)c3ccccc23)cc1 ; c1ccc(-c2c3ccccc3c(-c3ccc4oc5cc(-c6c7ccccc7c(-c7cccc8c7c7ccccc7n8-c7ccccc7)c7ccccc67)ccc5c4c3)c3ccccc23)cc1]

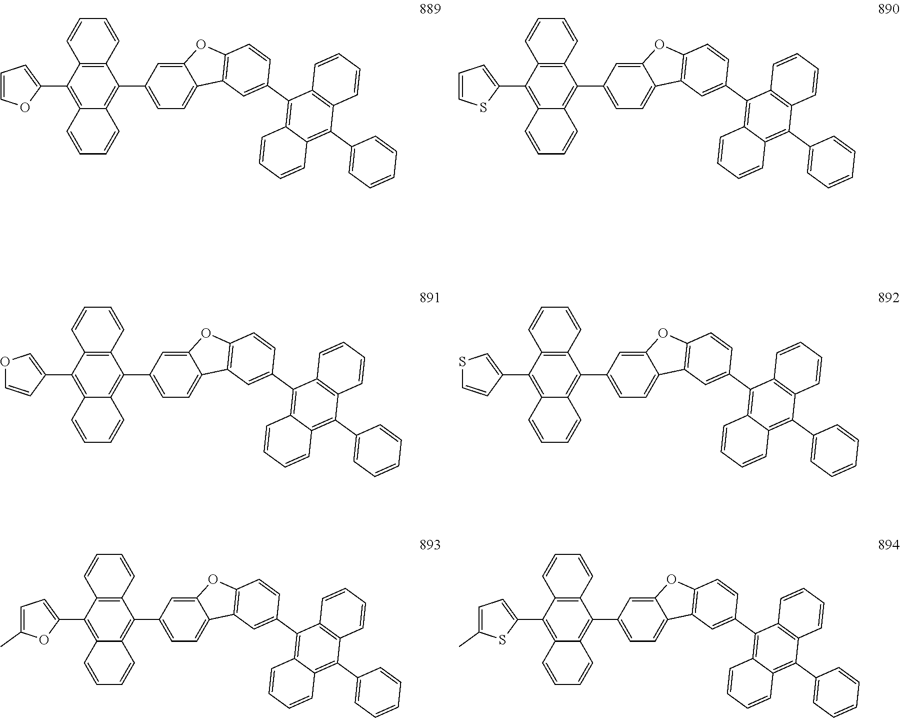

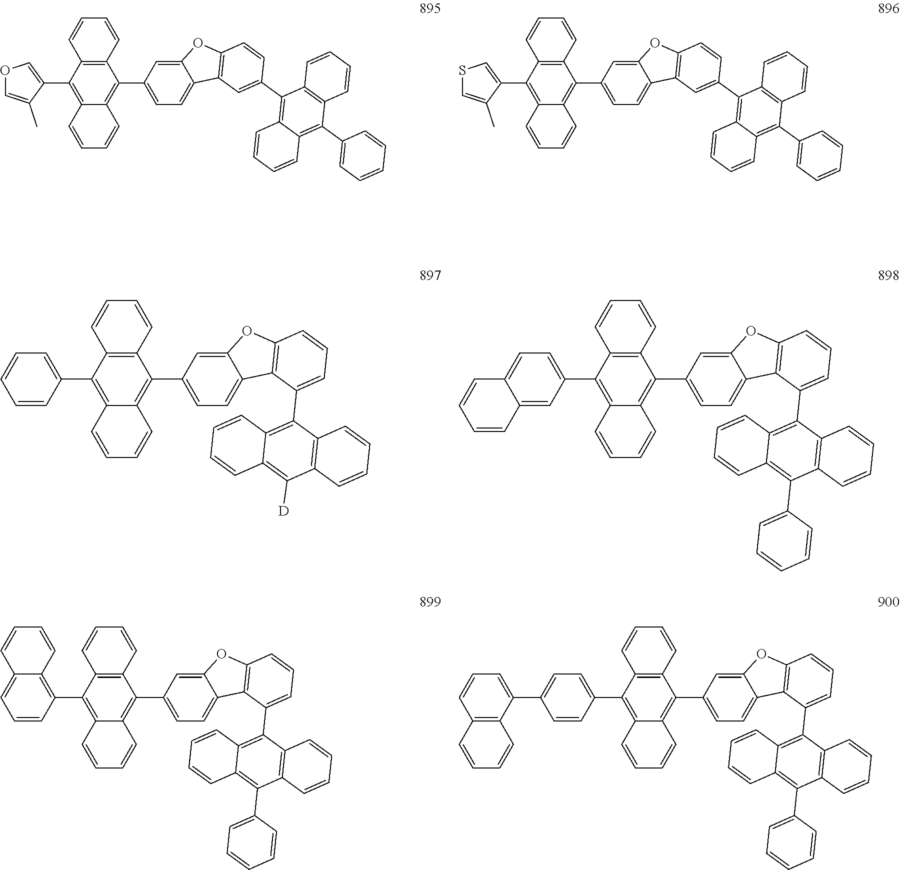

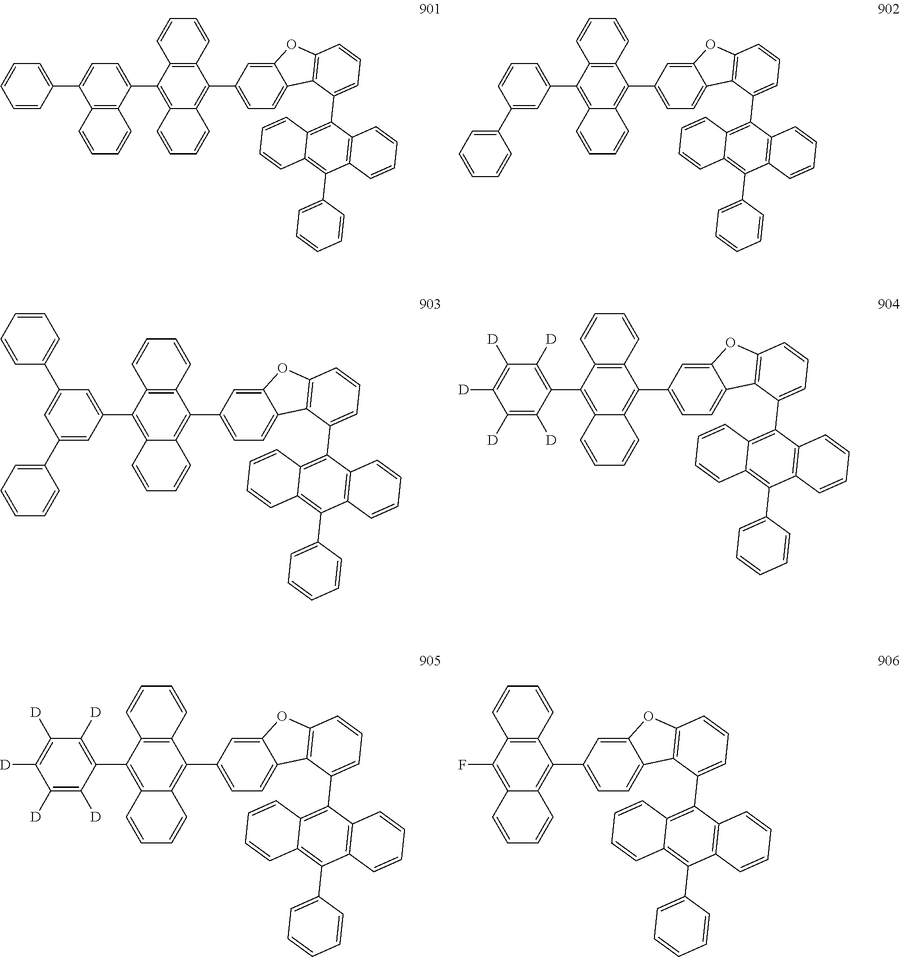

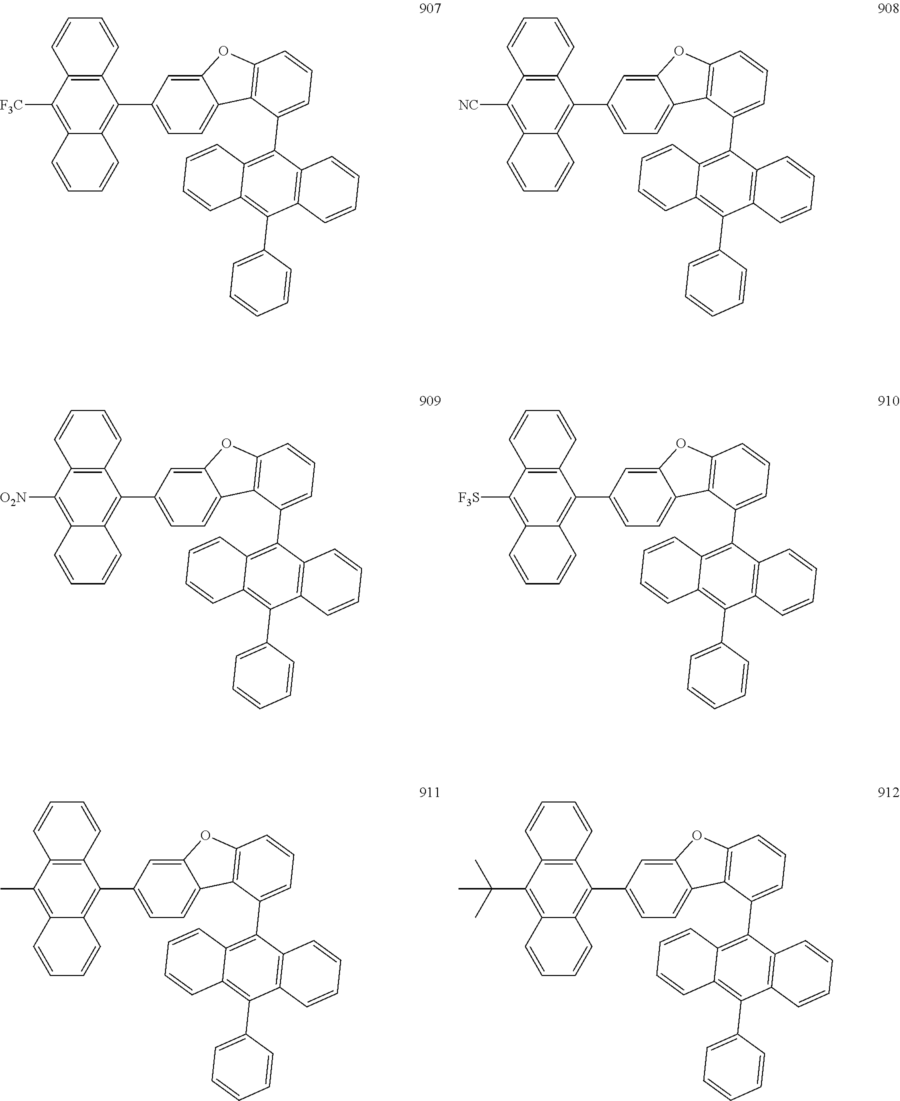

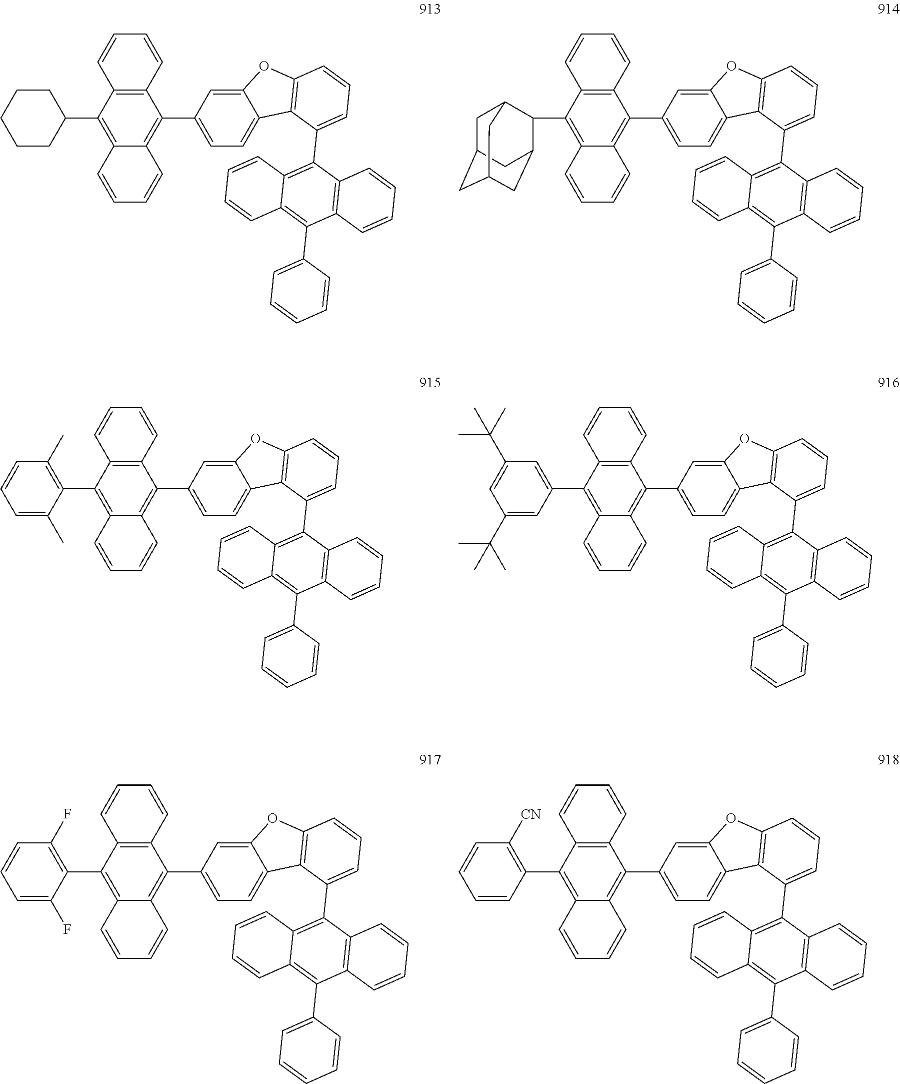

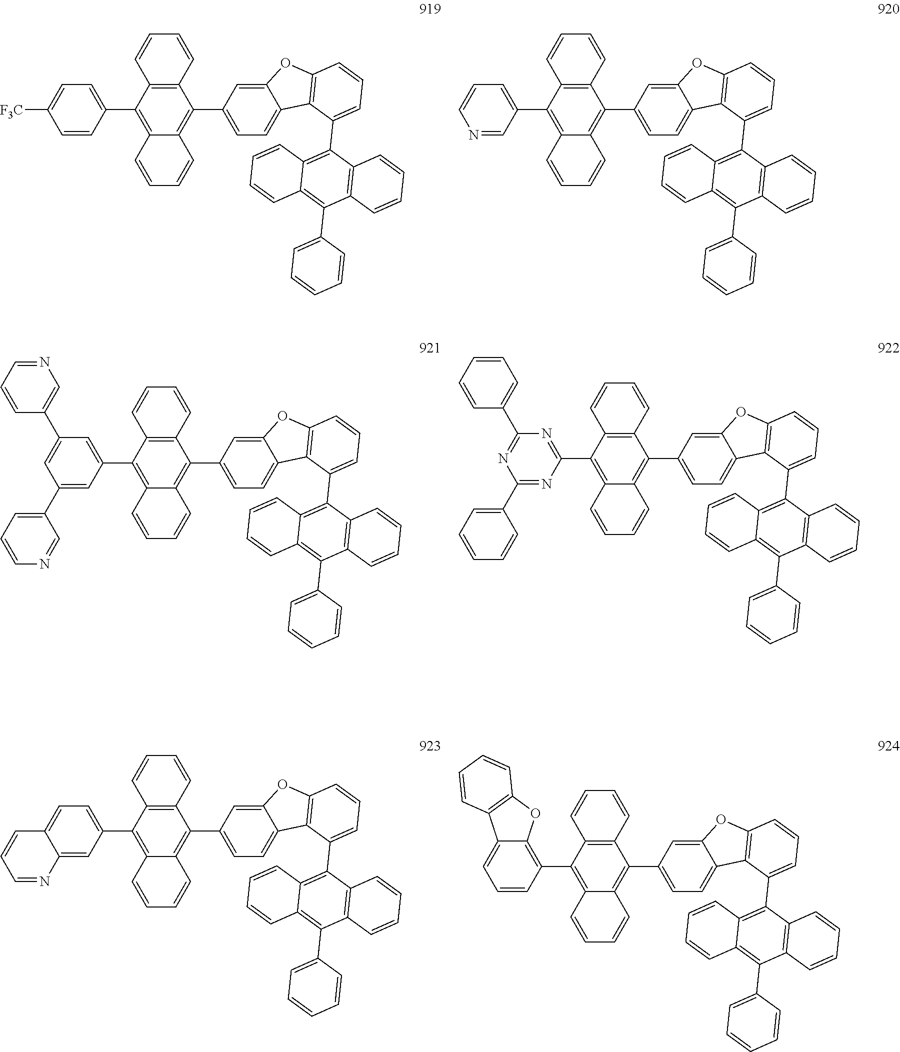

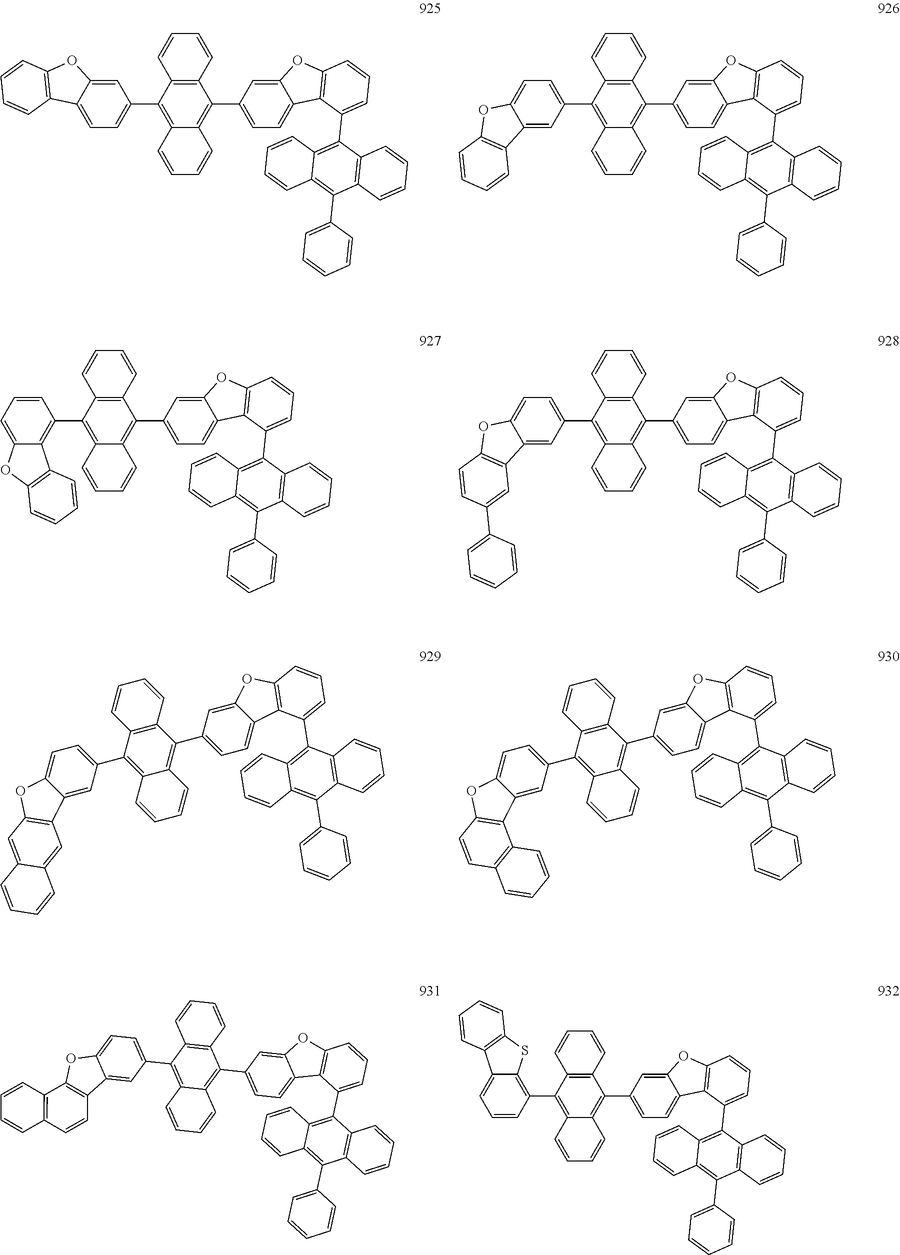

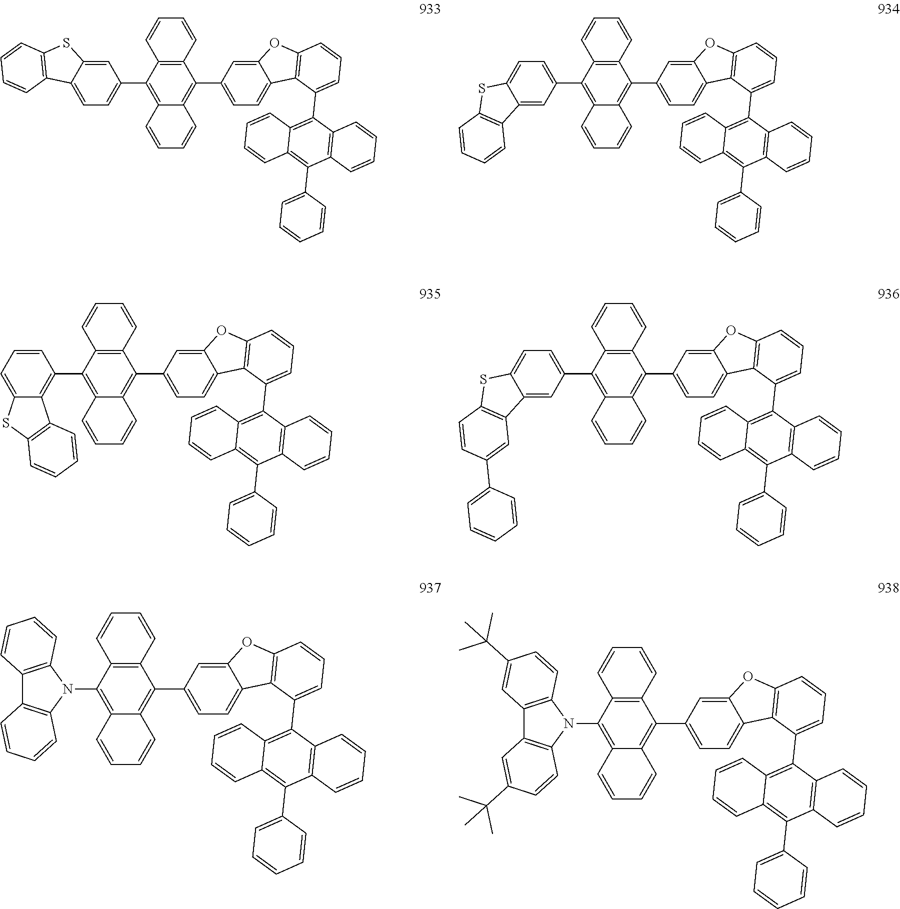

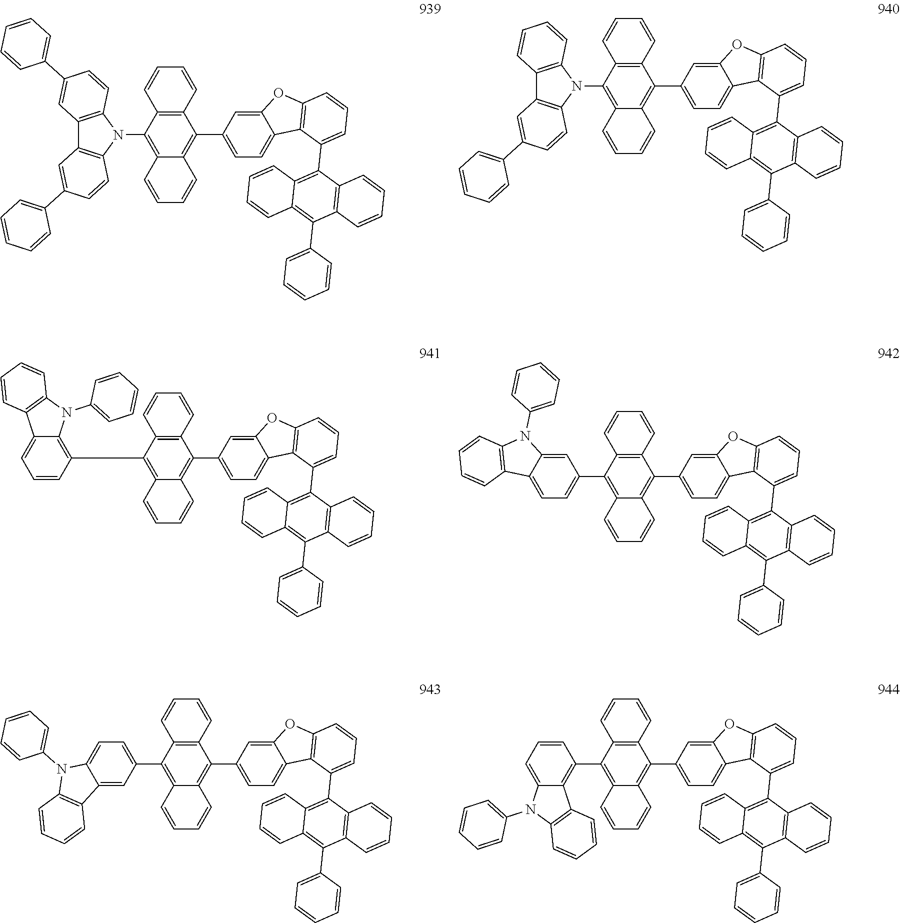

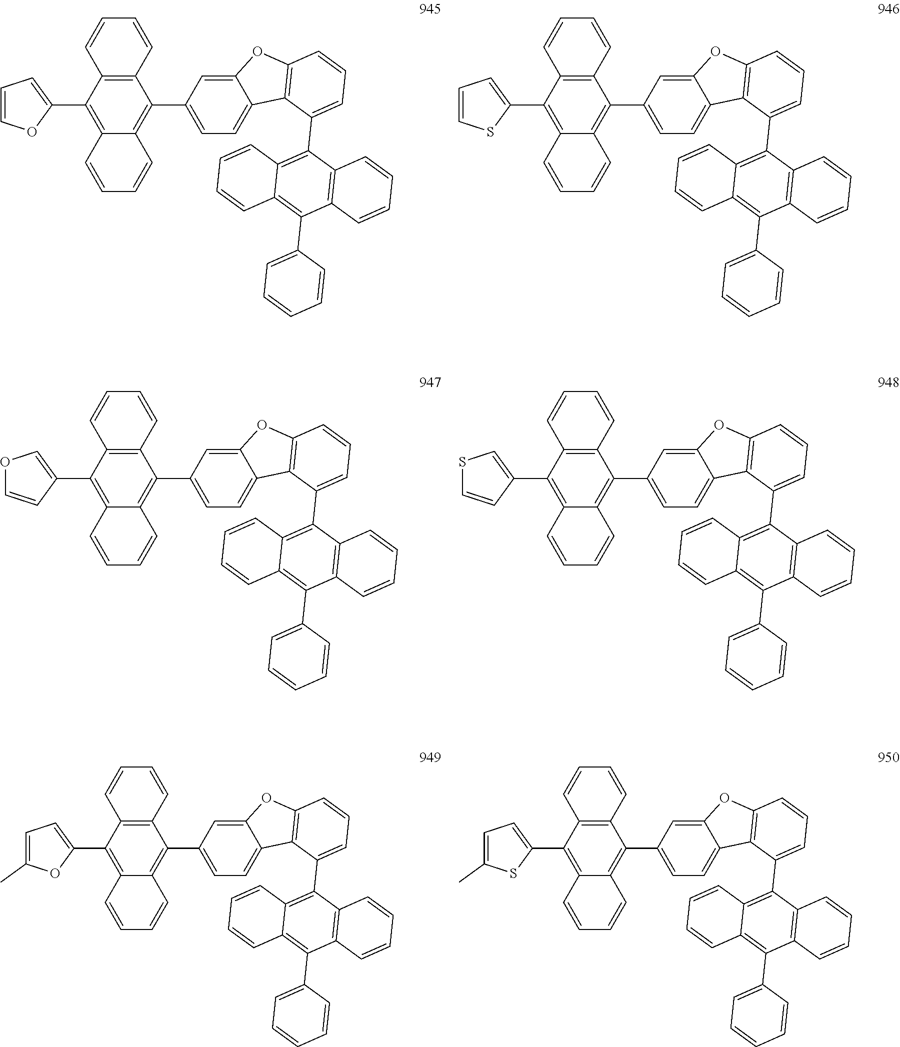

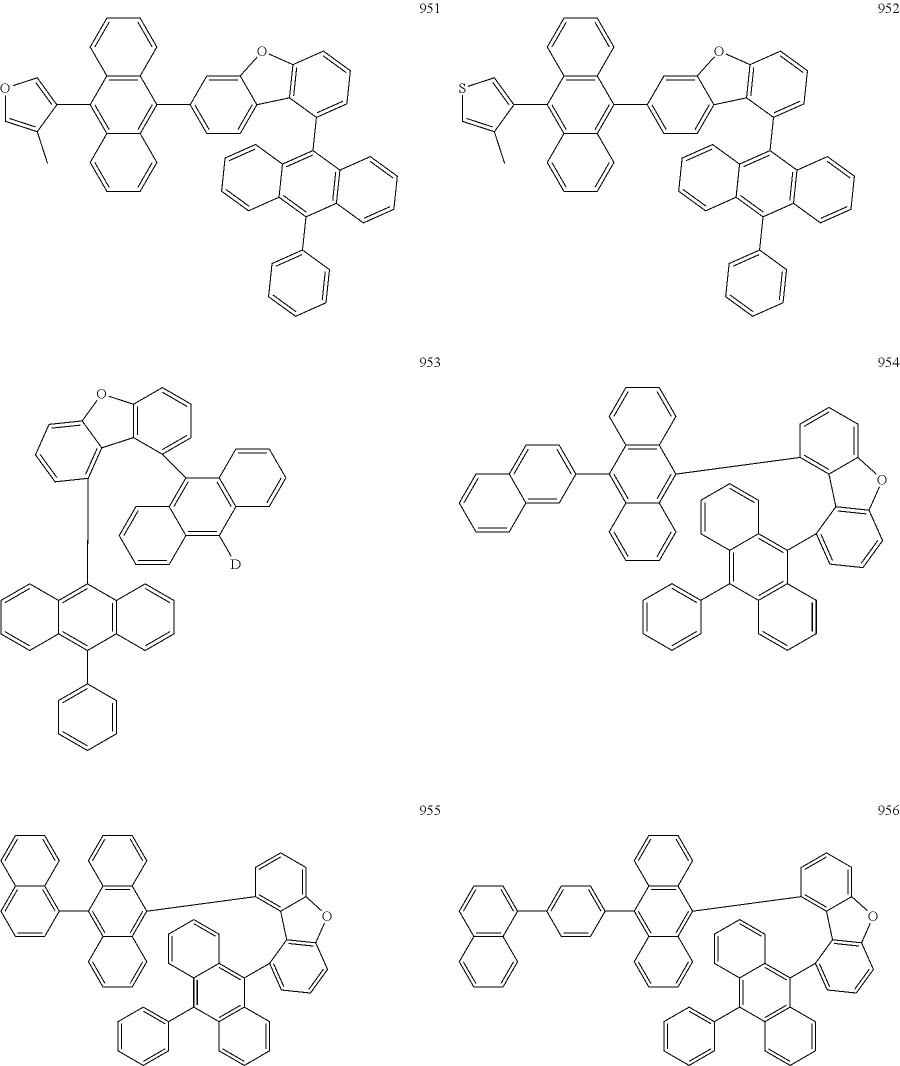

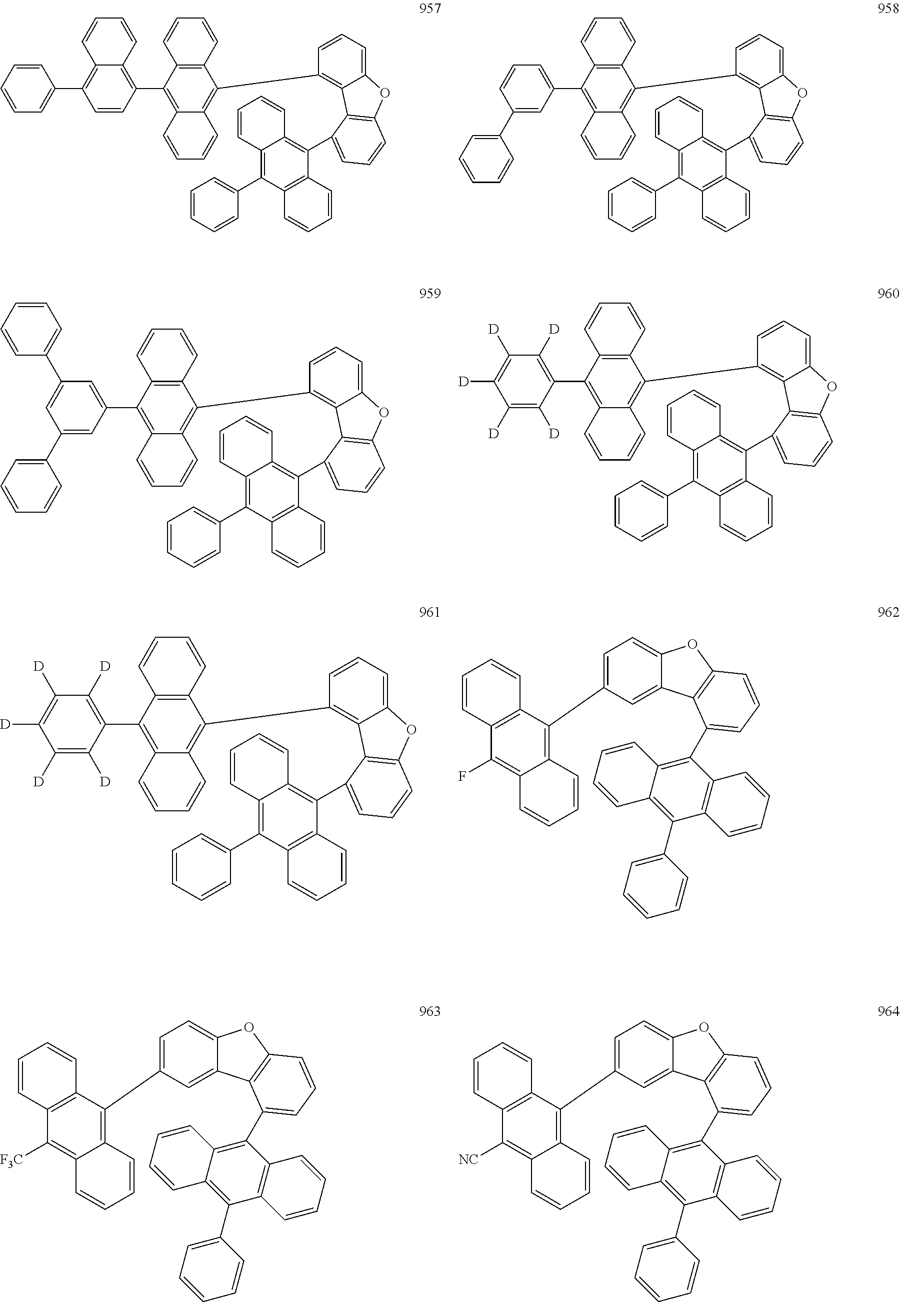

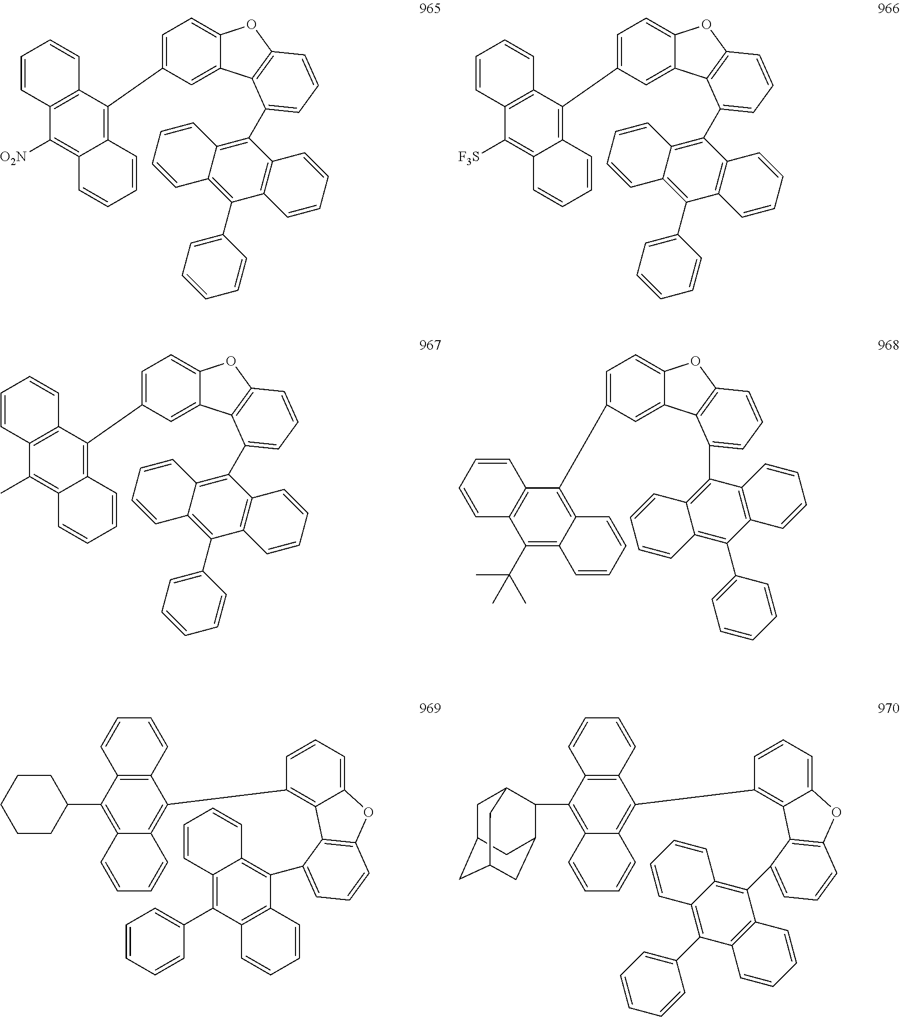

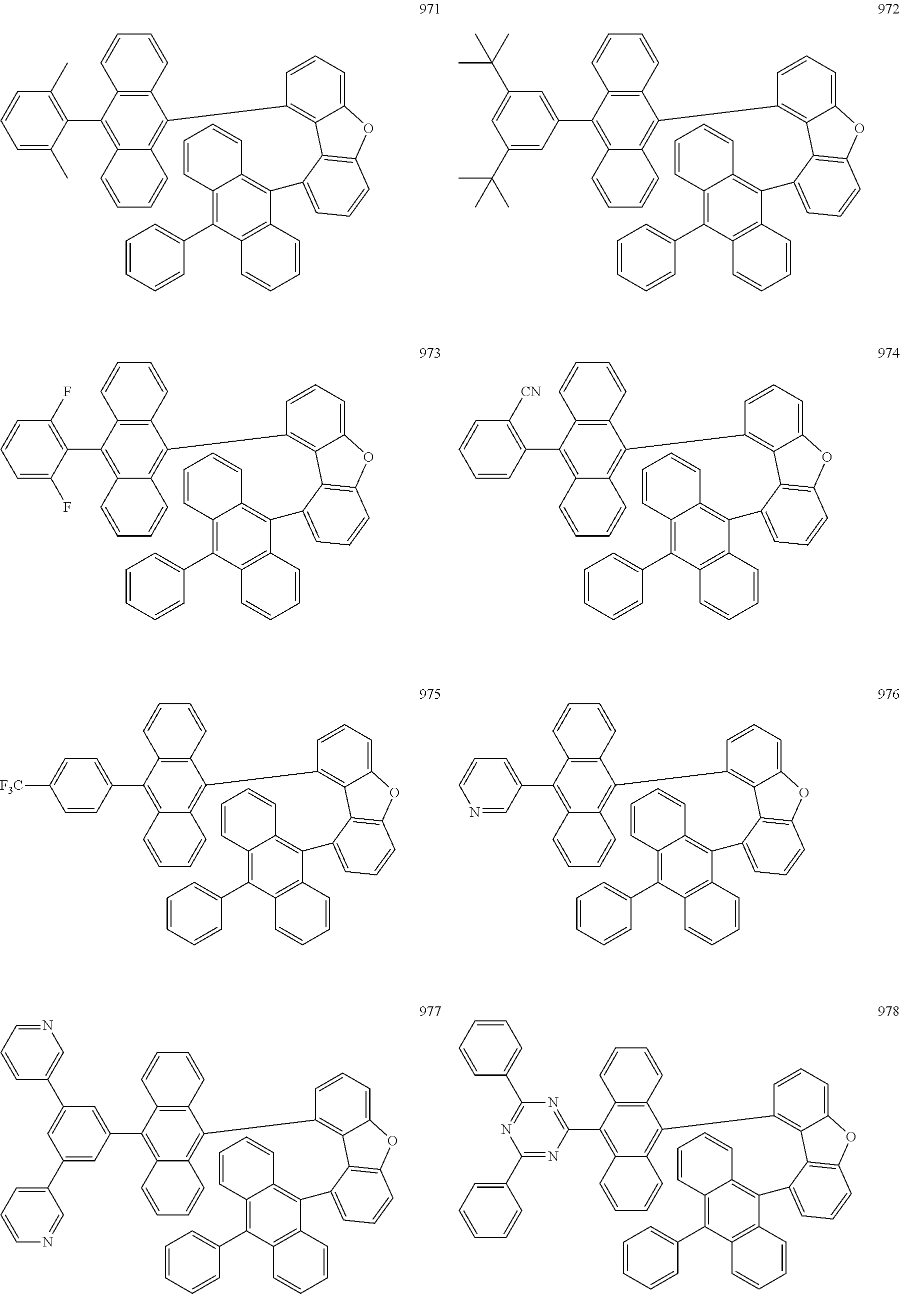

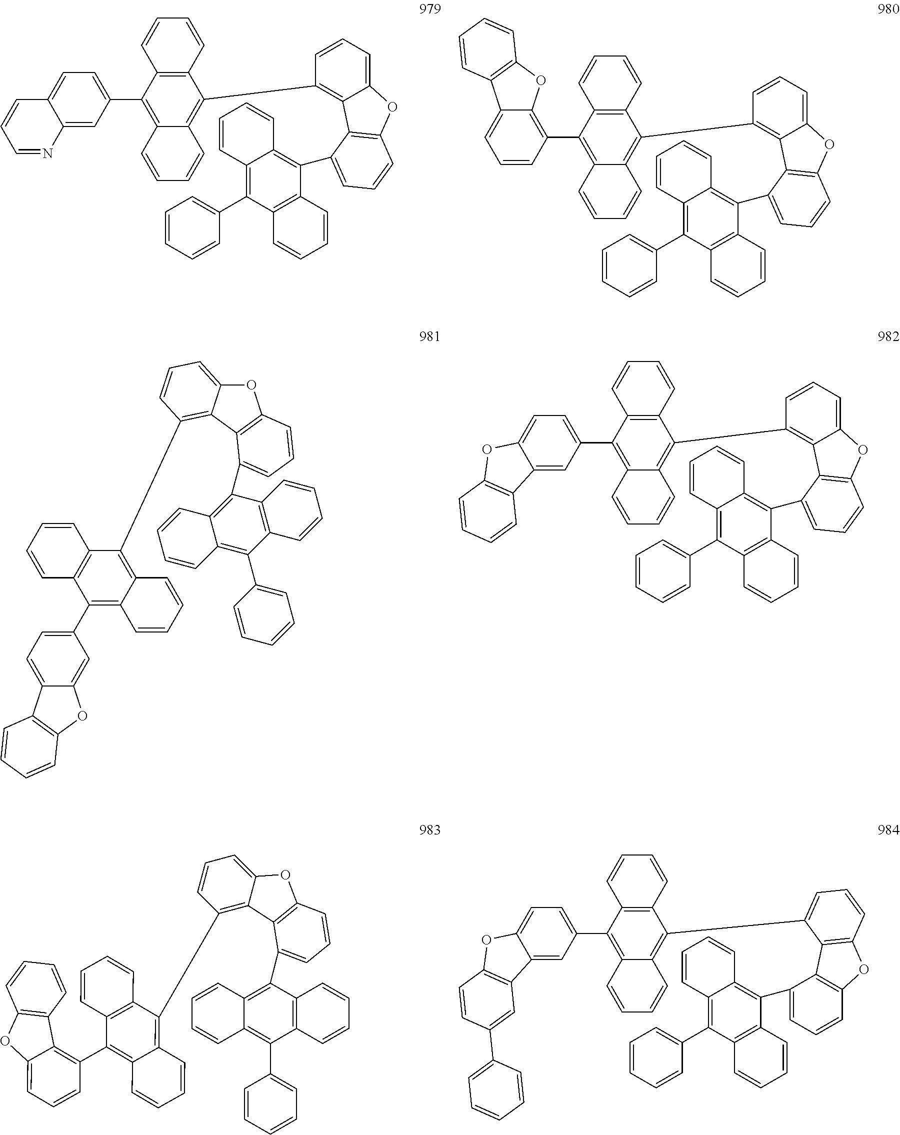

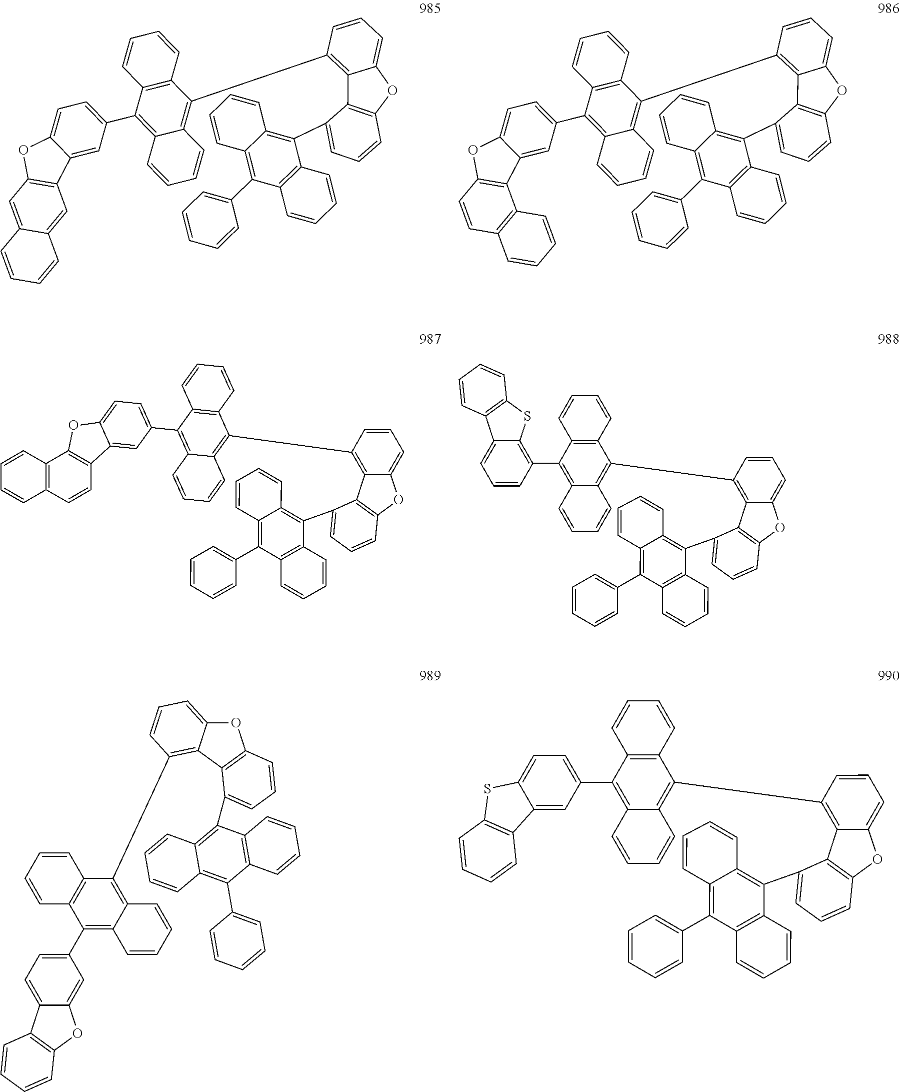

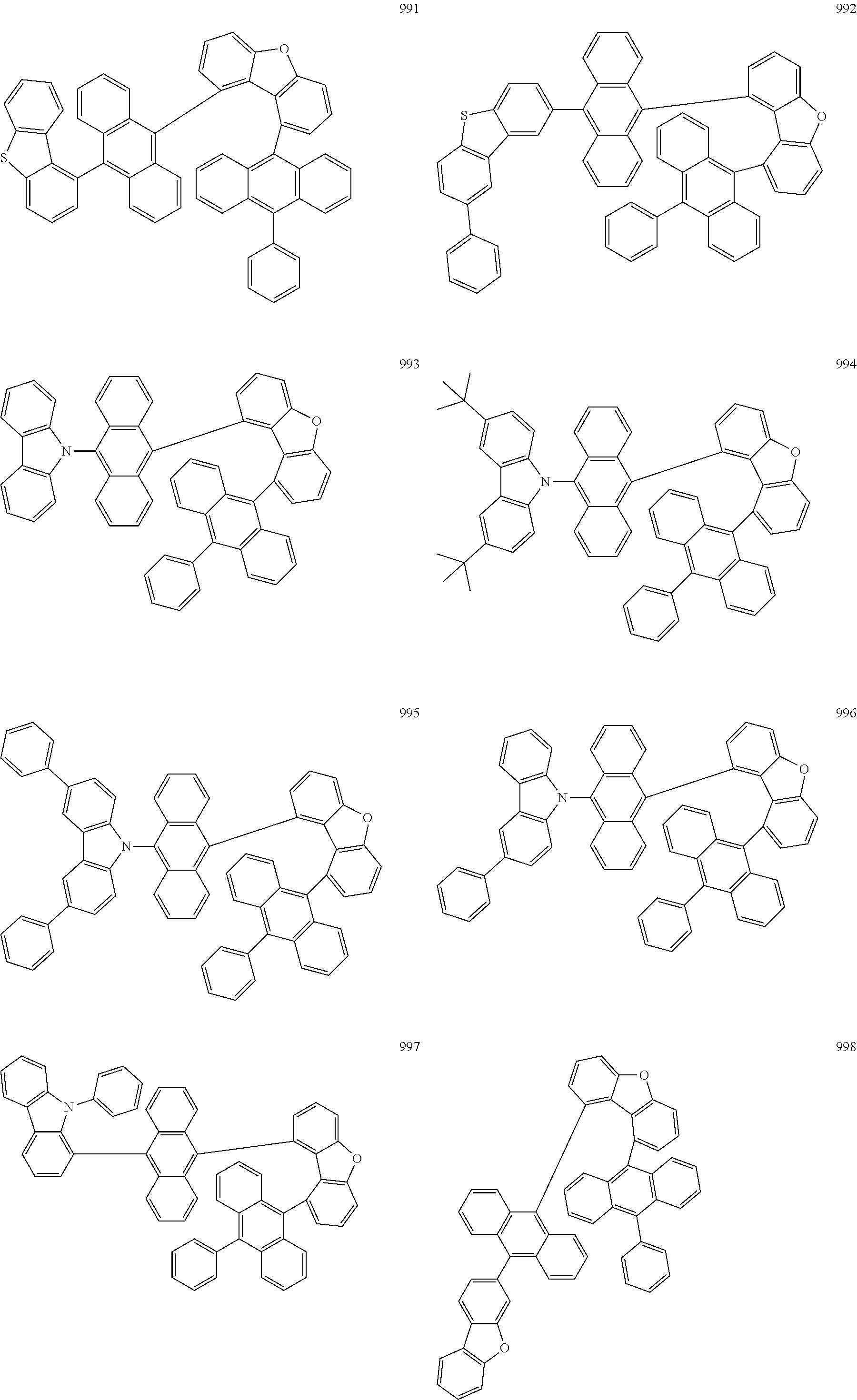

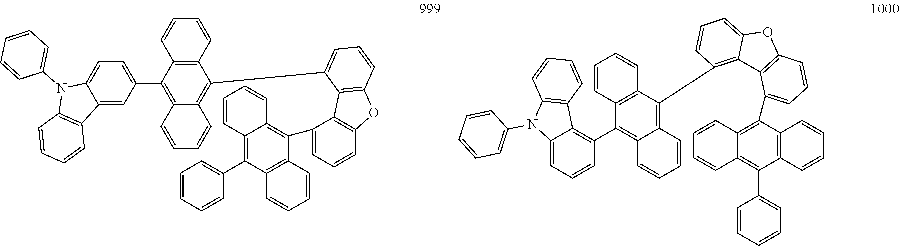 [SMILES: c1ccc(-c2c3ccccc3c(-c3cccc4oc5cccc(-c6c7ccccc7c(-c7ccc8c(c7)c7ccccc7n8-c7ccccc7)c7ccccc67)c5c34)c3ccccc23)cc1 ; c1ccc(-c2c3ccccc3c(-c3cccc4oc5cccc(-c6c7ccccc7c(-c7cccc8c7c7ccccc7n8-c7ccccc7)c7ccccc67)c5c34)c3ccccc23)cc1]

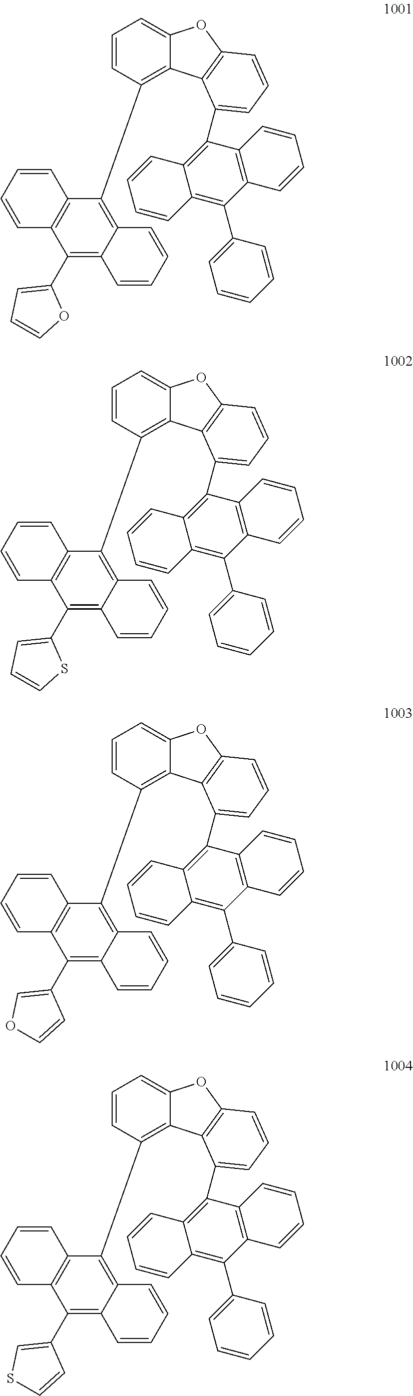 [SMILES: c1ccc(-c2c3ccccc3c(-c3cccc4oc5cccc(-c6c7ccccc7c(-c7ccco7)c7ccccc67)c5c34)c3ccccc23)cc1 ; c1ccc(-c2c3ccccc3c(-c3cccc4oc5cccc(-c6c7ccccc7c(-c7cccs7)c7ccccc67)c5c34)c3ccccc23)cc1 ; c1ccc(-c2c3ccccc3c(-c3cccc4oc5cccc(-c6c7ccccc7c(-c7ccoc7)c7ccccc67)c5c34)c3ccccc23)cc1 ; c1ccc(-c2c3ccccc3c(-c3cccc4oc5cccc(-c6c7ccccc7c(-c7ccsc7)c7ccccc67)c5c34)c3ccccc23)cc1]

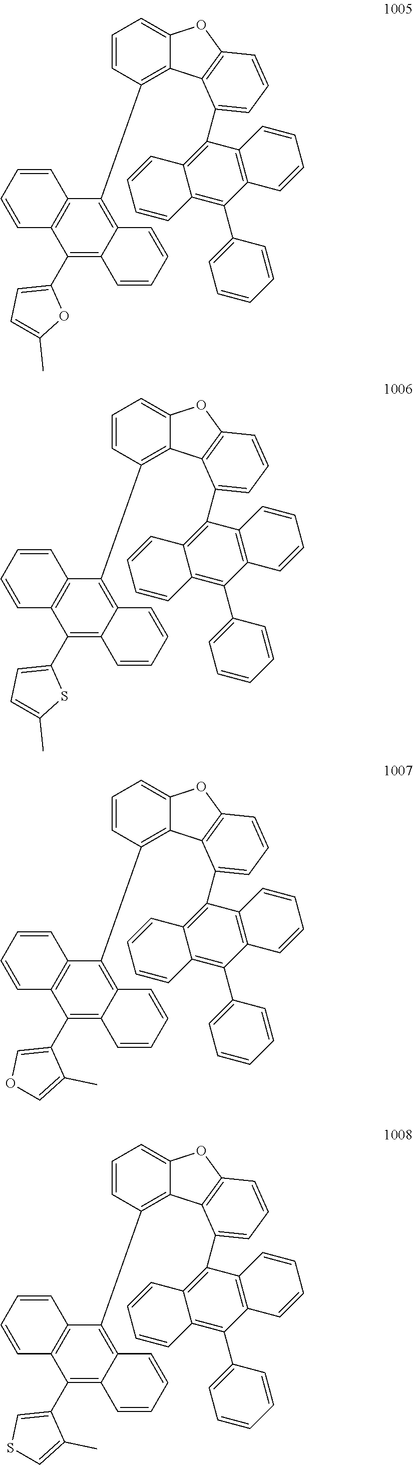 [SMILES: Cc1ccc(-c2c3ccccc3c(-c3cccc4oc5cccc(-c6c7ccccc7c(-c7ccccc7)c7ccccc67)c5c34)c3ccccc23)o1 ; Cc1ccc(-c2c3ccccc3c(-c3cccc4oc5cccc(-c6c7ccccc7c(-c7ccccc7)c7ccccc67)c5c34)c3ccccc23)s1 ; Cc1cocc1-c1c2ccccc2c(-c2cccc3oc4cccc(-c5c6ccccc6c(-c6ccccc6)c6ccccc56)c4c23)c2ccccc12 ; Cc1cscc1-c1c2ccccc2c(-c2cccc3oc4cccc(-c5c6ccccc6c(-c6ccccc6)c6ccccc56)c4c23)c2ccccc12]

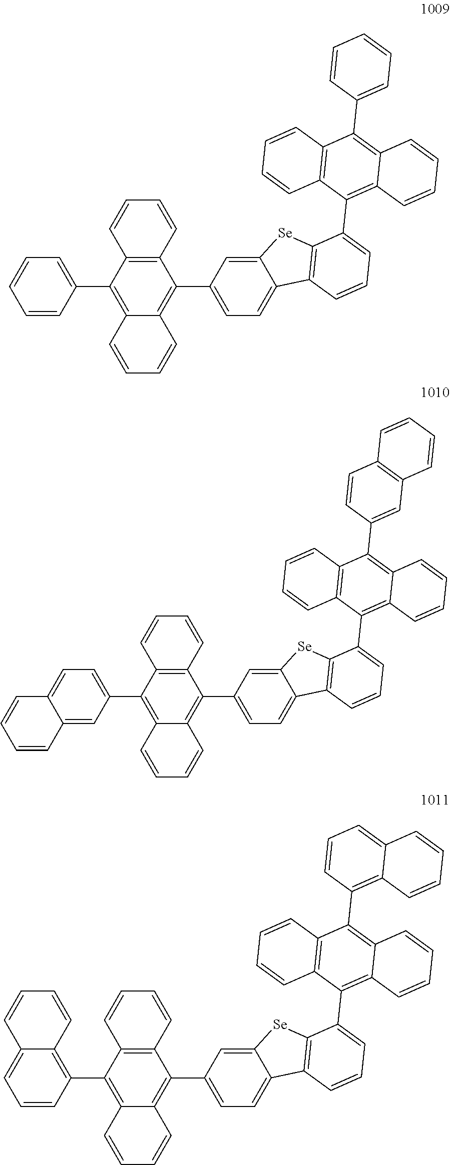 [SMILES: c1ccc(-c2c3ccccc3c(-c3ccc4c(c3)[se]c3c(-c5c6ccccc6c(-c6ccccc6)c6ccccc56)cccc34)c3ccccc23)cc1 ; c1ccc2cc(-c3c4ccccc4c(-c4ccc5c(c4)[se]c4c(-c6c7ccccc7c(-c7ccc8ccccc8c7)c7ccccc67)cccc45)c4ccccc34)ccc2c1 ; c1ccc2c(-c3c4ccccc4c(-c4ccc5c(c4)[se]c4c(-c6c7ccccc7c(-c7cccc8ccccc78)c7ccccc67)cccc45)c4ccccc34)cccc2c1]

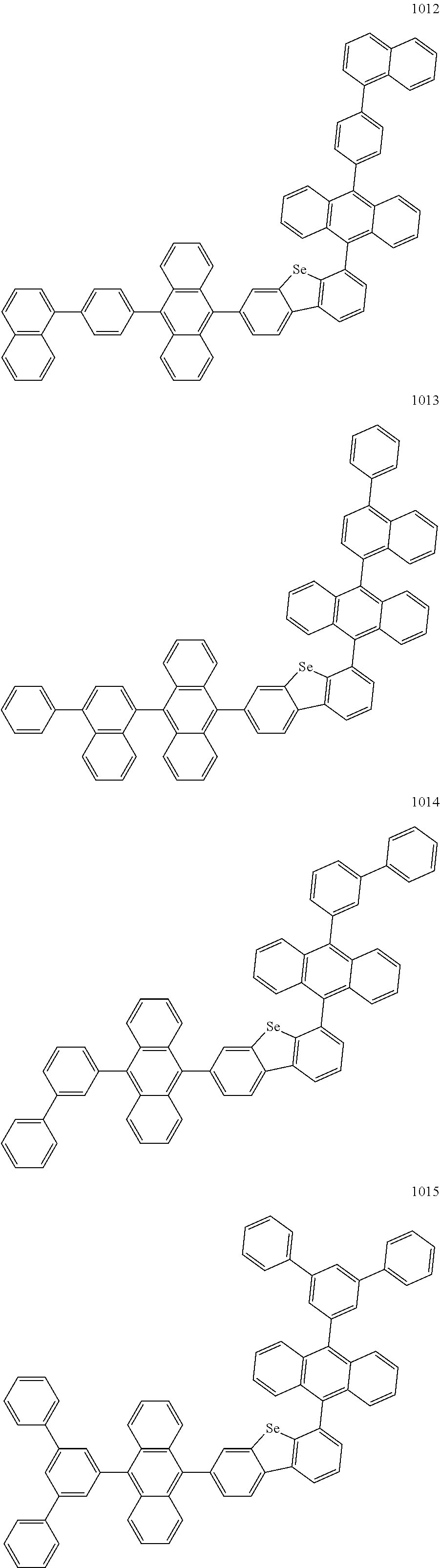

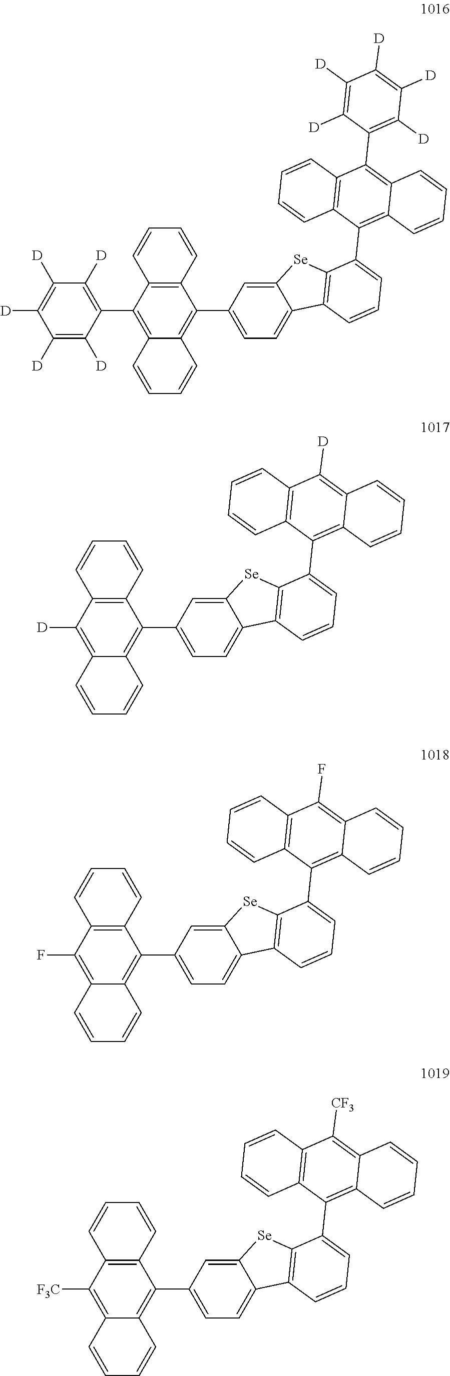 [SMILES: [2H]c1c([2H])c([2H])c(-c2c3ccccc3c(-c3ccc4c(c3)[se]c3c(-c5c6ccccc6c(-c6c([2H])c([2H])c([2H])c([2H])c6[2H])c6ccccc56)cccc34)c3ccccc23)c([2H])c1[2H] ; [2H]c1c2ccccc2c(-c2ccc3c(c2)[se]c2c(-c4c5ccccc5c([2H])c5ccccc45)cccc23)c2ccccc12 ; Fc1c2ccccc2c(-c2ccc3c(c2)[se]c2c(-c4c5ccccc5c(F)c5ccccc45)cccc23)c2ccccc12 ; FC(F)(F)c1c2ccccc2c(-c2ccc3c(c2)[se]c2c(-c4c5ccccc5c(C(F)(F)F)c5ccccc45)cccc23)c2ccccc12]

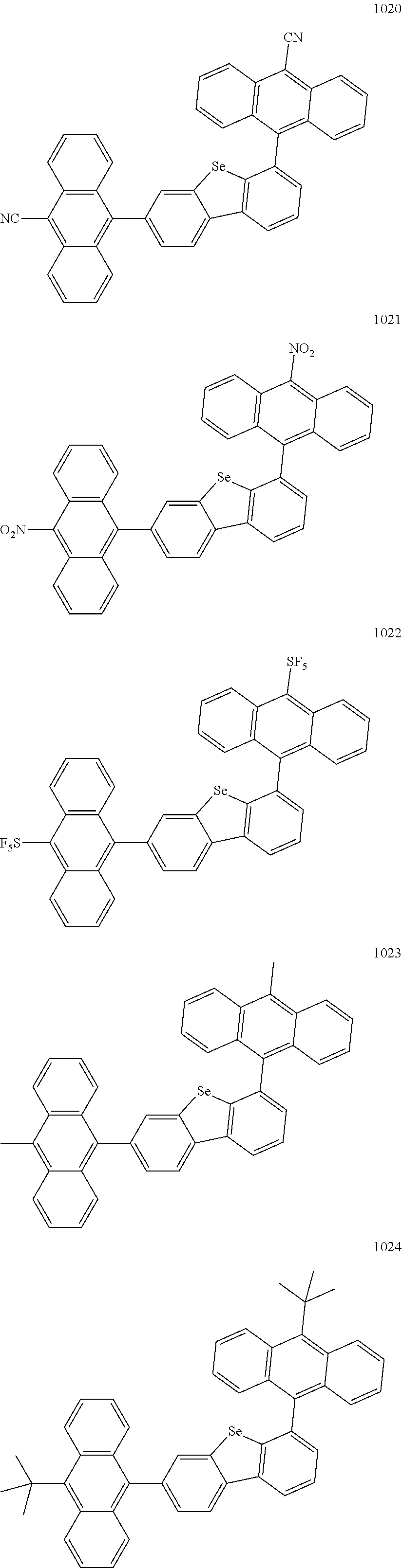

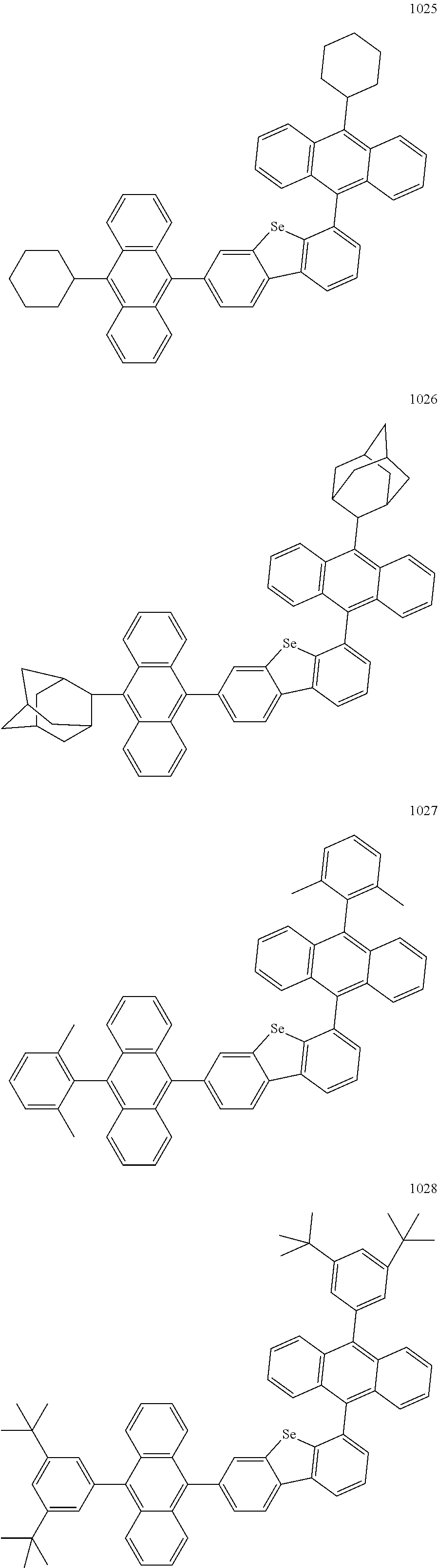 [SMILES: c1ccc2c(C3CCCCC3)c3ccccc3c(-c3ccc4c(c3)[se]c3c(-c5c6ccccc6c(C6CCCCC6)c6ccccc56)cccc34)c2c1 ; c1ccc2c(C3C4CC5CC(C4)CC3C5)c3ccccc3c(-c3ccc4c(c3)[se]c3c(-c5c6ccccc6c(C6C7CC8CC(C7)CC6C8)c6ccccc56)cccc34)c2c1 ; Cc1cccc(C)c1-c1c2ccccc2c(-c2ccc3c(c2)[se]c2c(-c4c5ccccc5c(-c5c(C)cccc5C)c5ccccc45)cccc23)c2ccccc12 ; CC(C)(C)c1cc(-c2c3ccccc3c(-c3ccc4c(c3)[se]c3c(-c5c6ccccc6c(-c6cc(C(C)(C)C)cc(C(C)(C)C)c6)c6ccccc56)cccc34)c3ccccc23)cc(C(C)(C)C)c1]

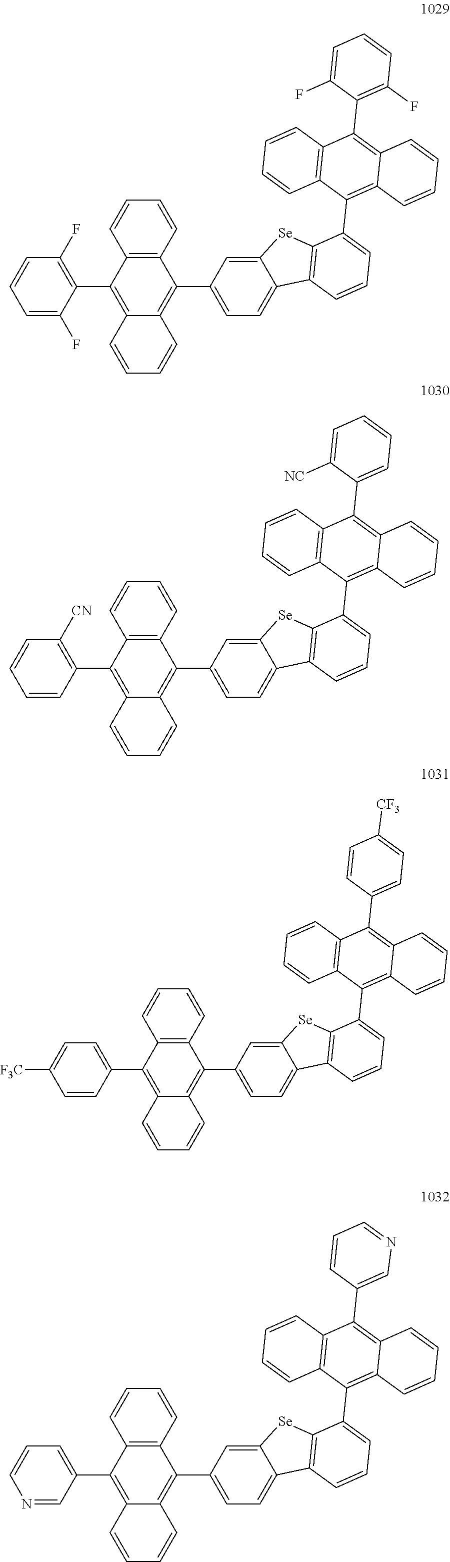 [SMILES: Fc1cccc(F)c1-c1c2ccccc2c(-c2ccc3c(c2)[se]c2c(-c4c5ccccc5c(-c5c(F)cccc5F)c5ccccc45)cccc23)c2ccccc12 ; N#Cc1ccccc1-c1c2ccccc2c(-c2ccc3c(c2)[se]c2c(-c4c5ccccc5c(-c5ccccc5C#N)c5ccccc45)cccc23)c2ccccc12 ; FC(F)(F)c1ccc(-c2c3ccccc3c(-c3ccc4c(c3)[se]c3c(-c5c6ccccc6c(-c6ccc(C(F)(F)F)cc6)c6ccccc56)cccc34)c3ccccc23)cc1 ; c1cncc(-c2c3ccccc3c(-c3ccc4c(c3)[se]c3c(-c5c6ccccc6c(-c6cccnc6)c6ccccc56)cccc34)c3ccccc23)c1]

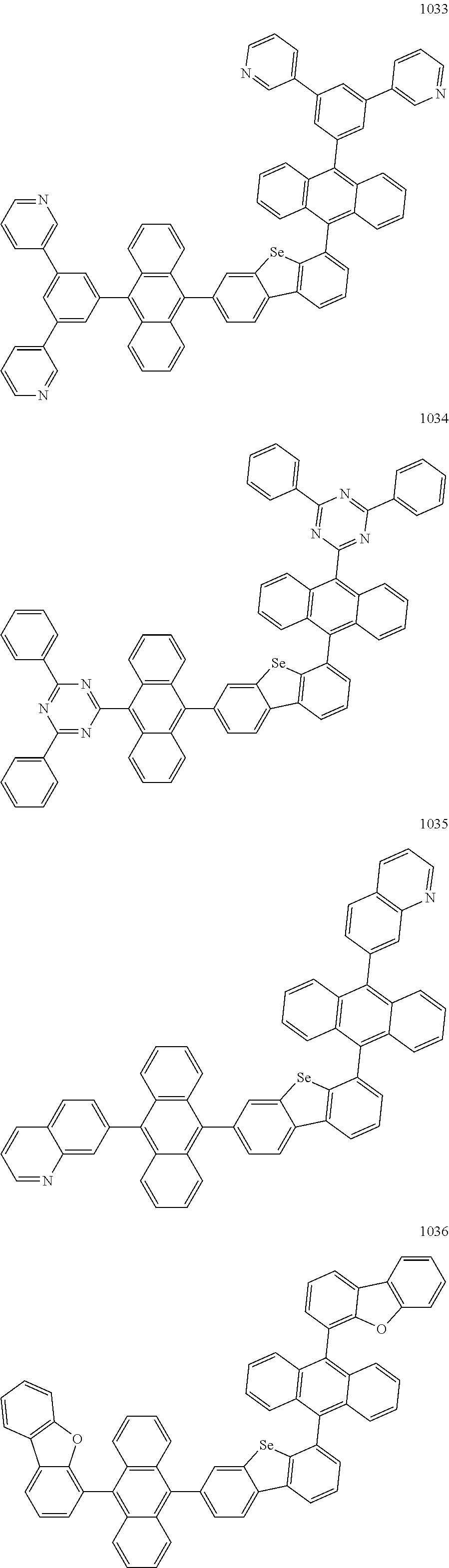

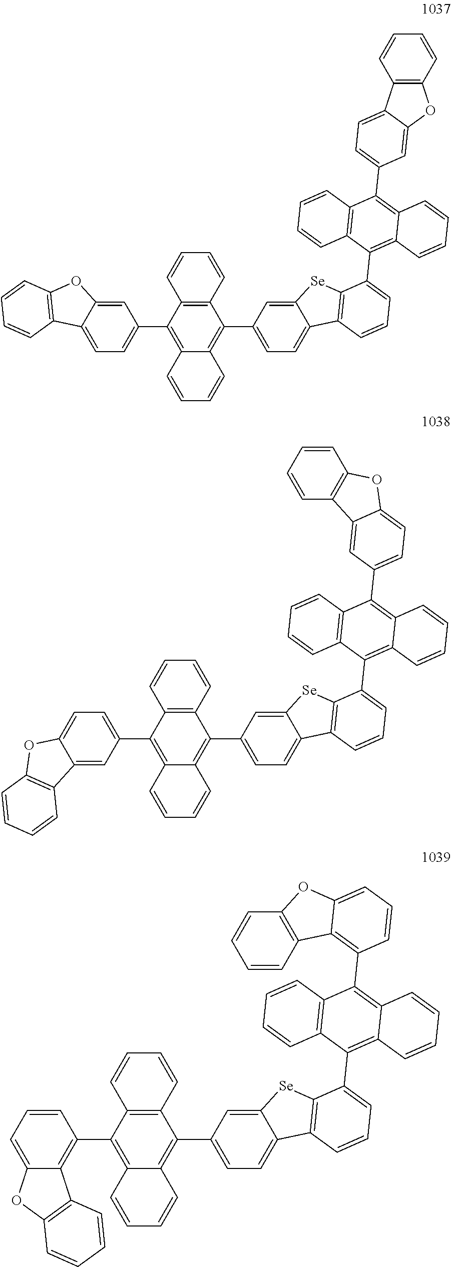 [SMILES: c1ccc2c(c1)oc1cc(-c3c4ccccc4c(-c4ccc5c(c4)[se]c4c(-c6c7ccccc7c(-c7ccc8c(c7)oc7ccccc78)c7ccccc67)cccc45)c4ccccc34)ccc12 ; c1ccc2c(c1)oc1ccc(-c3c4ccccc4c(-c4ccc5c(c4)[se]c4c(-c6c7ccccc7c(-c7ccc8oc9ccccc9c8c7)c7ccccc67)cccc45)c4ccccc34)cc12 ; c1ccc2c(c1)oc1cccc(-c3c4ccccc4c(-c4ccc5c(c4)[se]c4c(-c6c7ccccc7c(-c7cccc8oc9ccccc9c78)c7ccccc67)cccc45)c4ccccc34)c12]

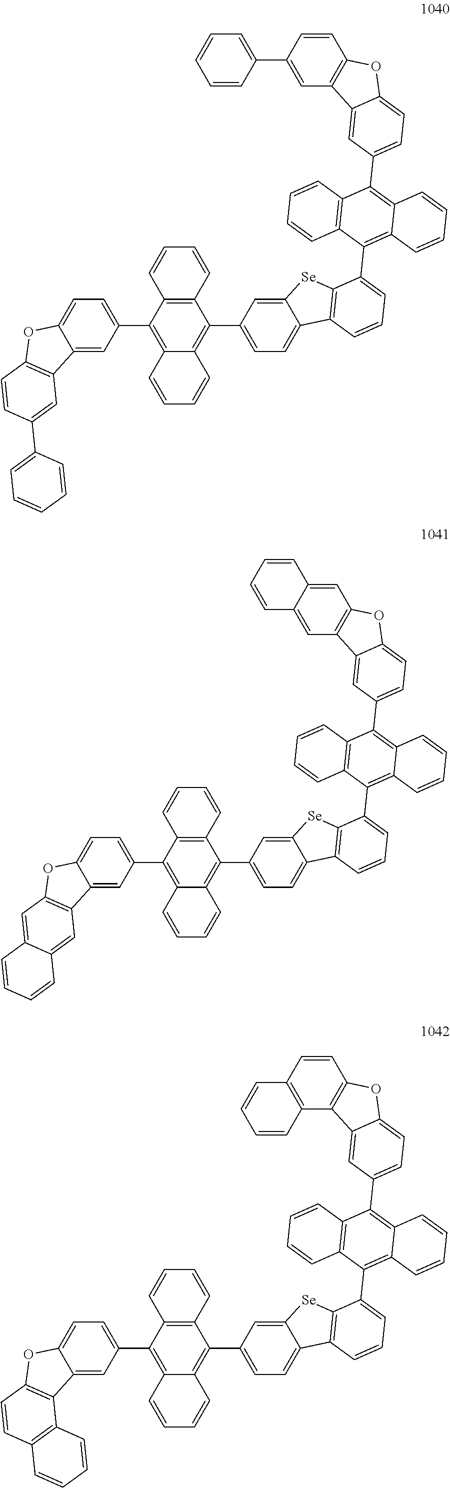 [SMILES: c1ccc(-c2ccc3oc4ccc(-c5c6ccccc6c(-c6ccc7c(c6)[se]c6c(-c8c9ccccc9c(-c9ccc%10oc%11ccc(-c%12ccccc%12)cc%11c%10c9)c9ccccc89)cccc67)c6ccccc56)cc4c3c2)cc1 ; c1ccc2cc3c(cc2c1)oc1ccc(-c2c4ccccc4c(-c4ccc5c(c4)[se]c4c(-c6c7ccccc7c(-c7ccc8oc9cc%10ccccc%10cc9c8c7)c7ccccc67)cccc45)c4ccccc24)cc13 ; c1ccc2c(c1)ccc1oc3ccc(-c4c5ccccc5c(-c5ccc6c(c5)[se]c5c(-c7c8ccccc8c(-c8ccc9oc%10ccc%11ccccc%11c%10c9c8)c8ccccc78)cccc56)c5ccccc45)cc3c12]

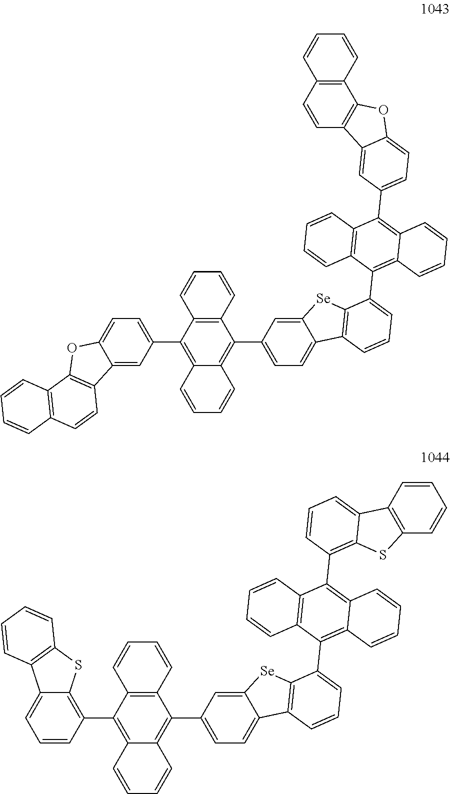 [SMILES: c1ccc2c(c1)ccc1c3cc(-c4c5ccccc5c(-c5ccc6c(c5)[se]c5c(-c7c8ccccc8c(-c8ccc9oc%10c%11ccccc%11ccc%10c9c8)c8ccccc78)cccc56)c5ccccc45)ccc3oc21 ; c1ccc2c(c1)sc1c(-c3c4ccccc4c(-c4ccc5c(c4)[se]c4c(-c6c7ccccc7c(-c7cccc8c7sc7ccccc78)c7ccccc67)cccc45)c4ccccc34)cccc12]

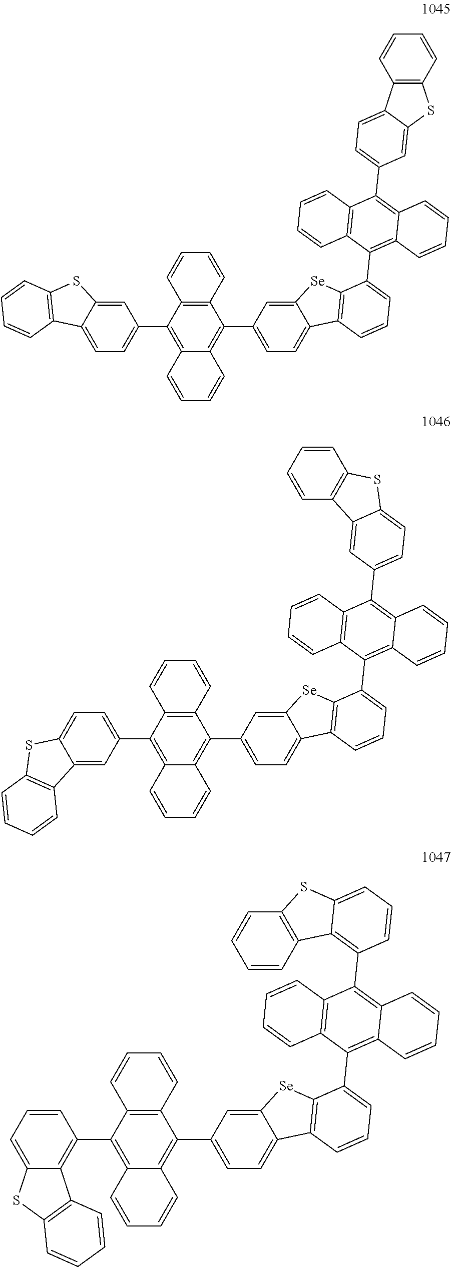 [SMILES: c1ccc2c(c1)sc1cc(-c3c4ccccc4c(-c4ccc5c(c4)[se]c4c(-c6c7ccccc7c(-c7ccc8c(c7)sc7ccccc78)c7ccccc67)cccc45)c4ccccc34)ccc12 ; c1ccc2c(c1)sc1ccc(-c3c4ccccc4c(-c4ccc5c(c4)[se]c4c(-c6c7ccccc7c(-c7ccc8sc9ccccc9c8c7)c7ccccc67)cccc45)c4ccccc34)cc12 ; c1ccc2c(c1)sc1cccc(-c3c4ccccc4c(-c4ccc5c(c4)[se]c4c(-c6c7ccccc7c(-c7cccc8sc9ccccc9c78)c7ccccc67)cccc45)c4ccccc34)c12]

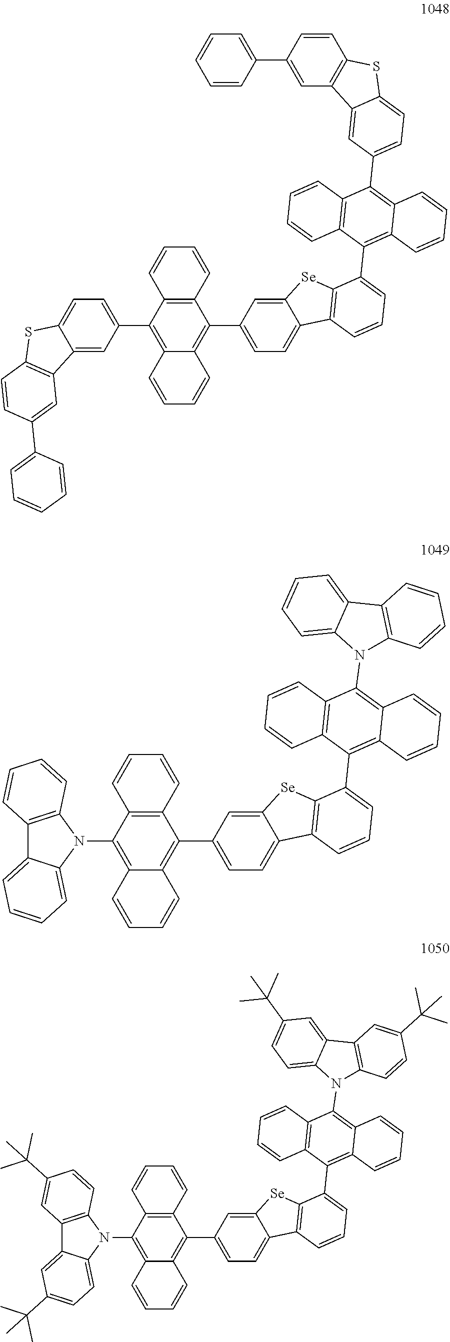 [SMILES: c1ccc(-c2ccc3sc4ccc(-c5c6ccccc6c(-c6ccc7c(c6)[se]c6c(-c8c9ccccc9c(-c9ccc%10sc%11ccc(-c%12ccccc%12)cc%11c%10c9)c9ccccc89)cccc67)c6ccccc56)cc4c3c2)cc1 ; c1ccc2c(-n3c4ccccc4c4ccccc43)c3ccccc3c(-c3ccc4c(c3)[se]c3c(-c5c6ccccc6c(-n6c7ccccc7c7ccccc76)c6ccccc56)cccc34)c2c1 ; CC(C)(C)c1ccc2c(c1)c1cc(C(C)(C)C)ccc1n2-c1c2ccccc2c(-c2ccc3c(c2)[se]c2c(-c4c5ccccc5c(-n5c6ccc(C(C)(C)C)cc6c6cc(C(C)(C)C)ccc65)c5ccccc45)cccc23)c2ccccc12]

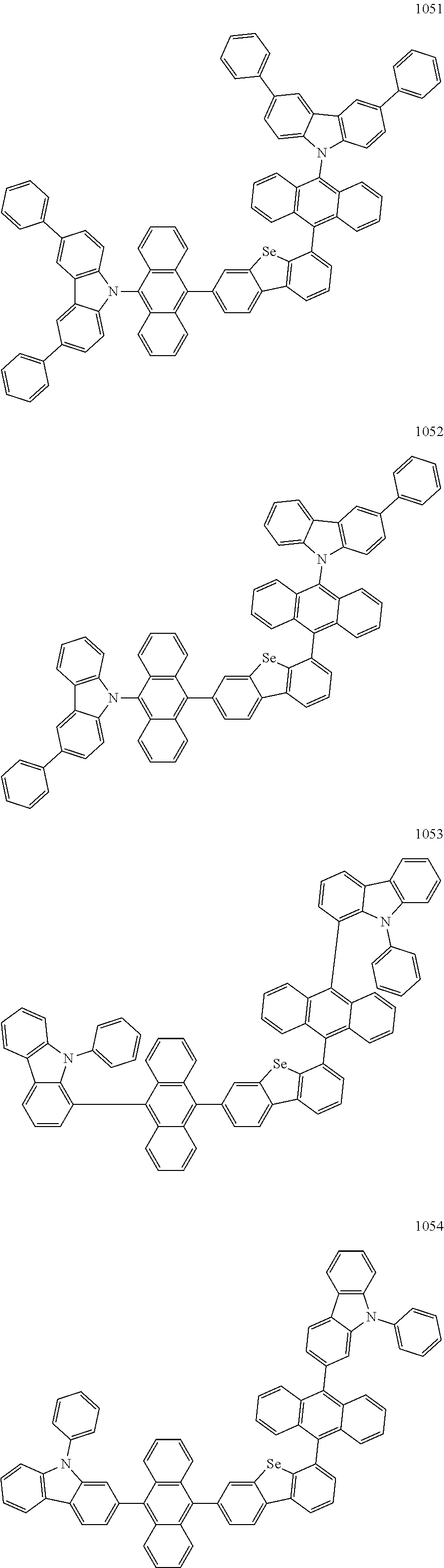

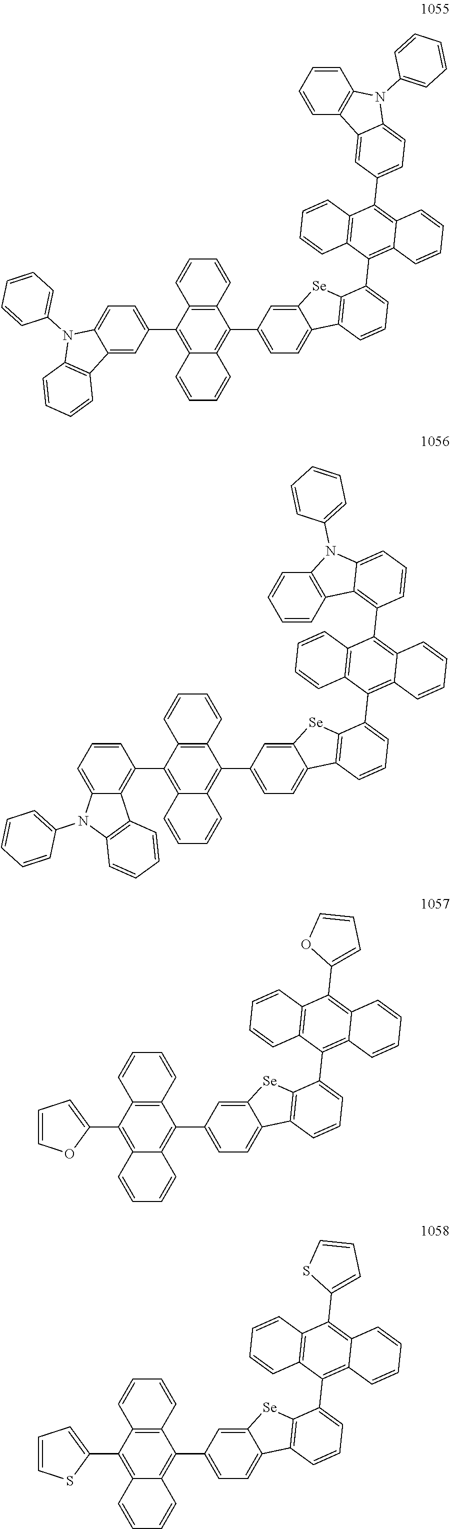

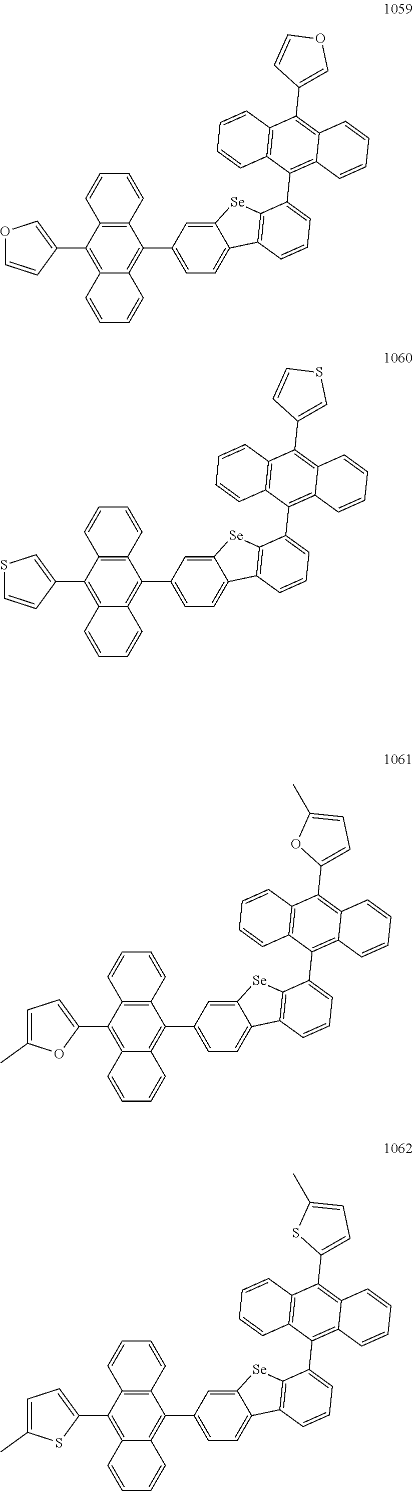

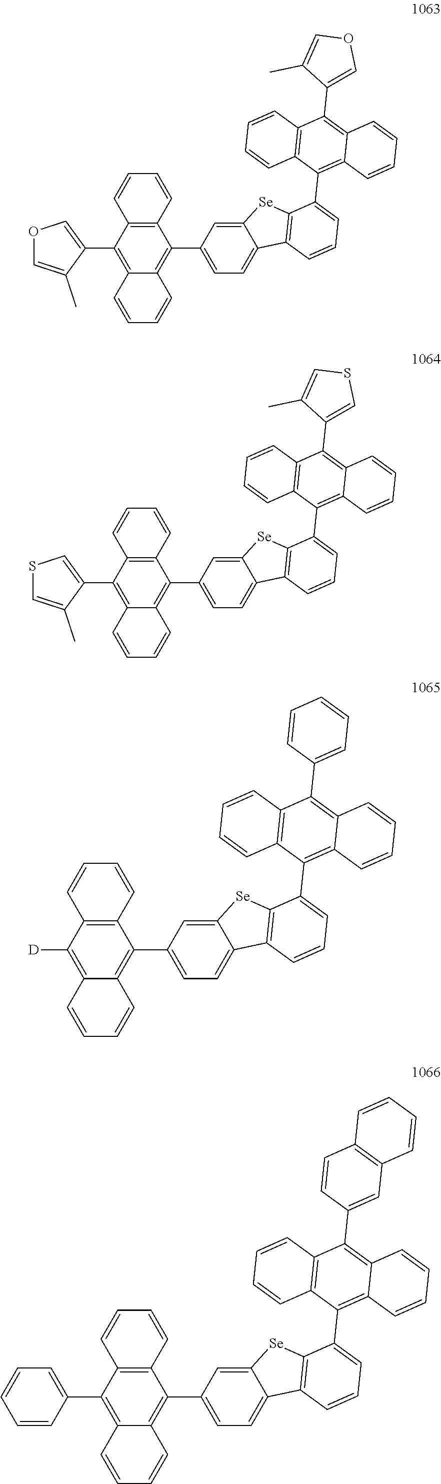

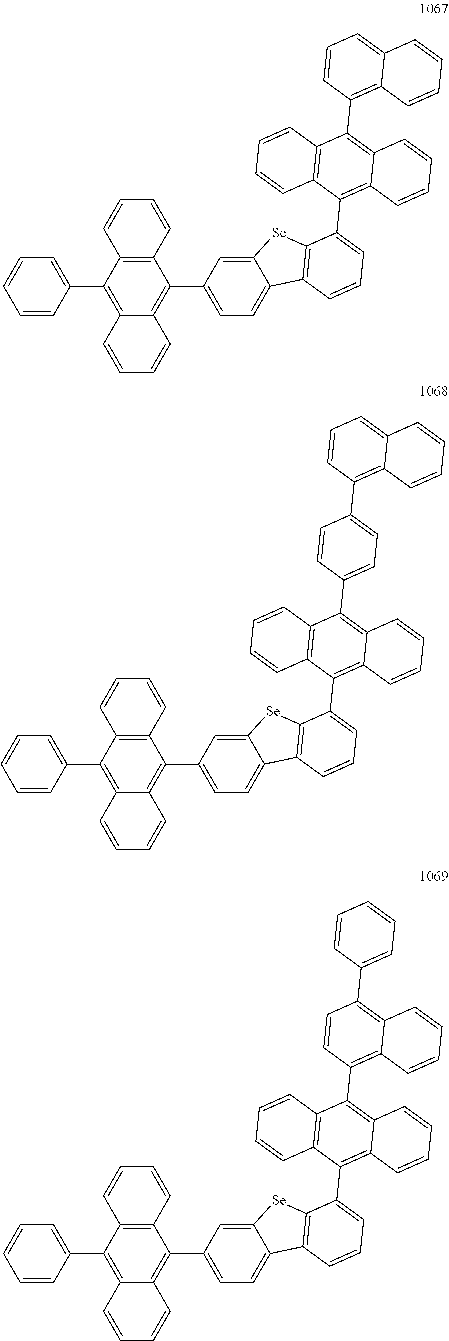

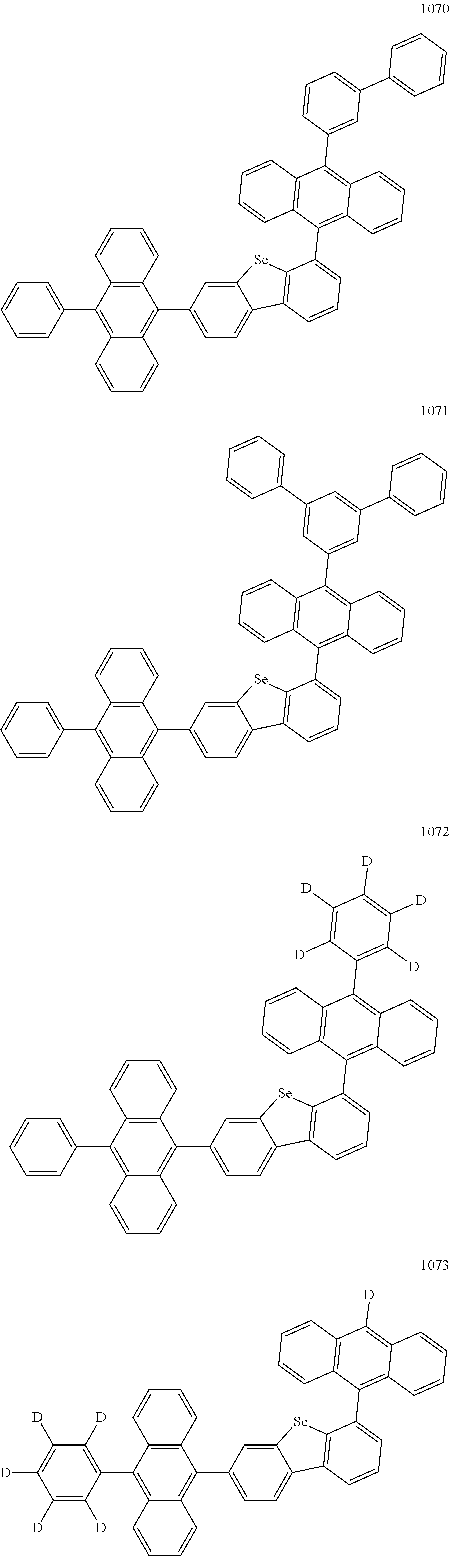

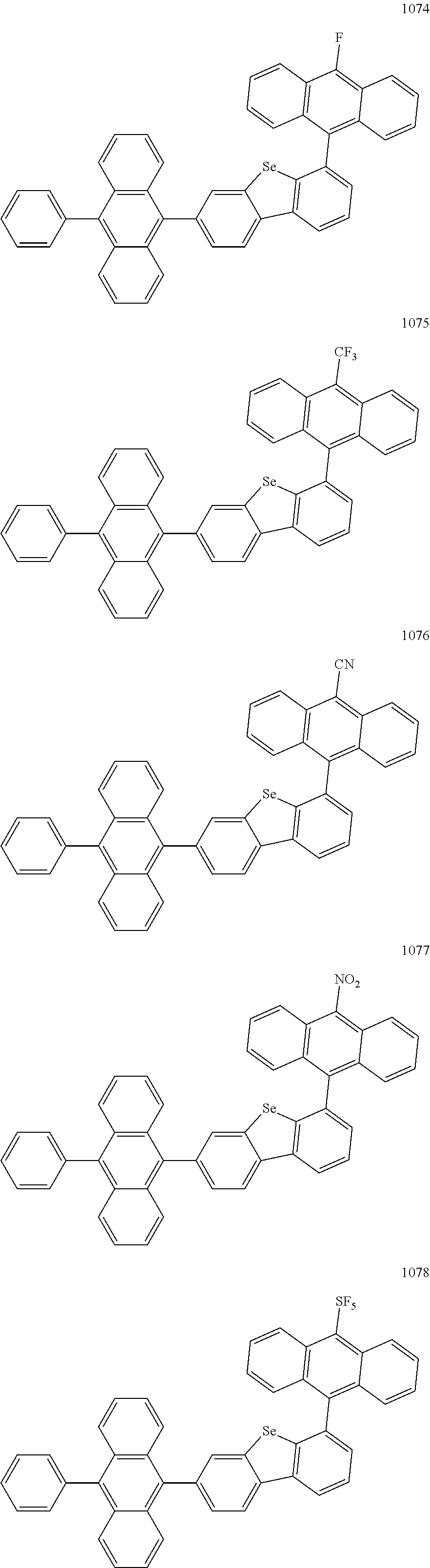

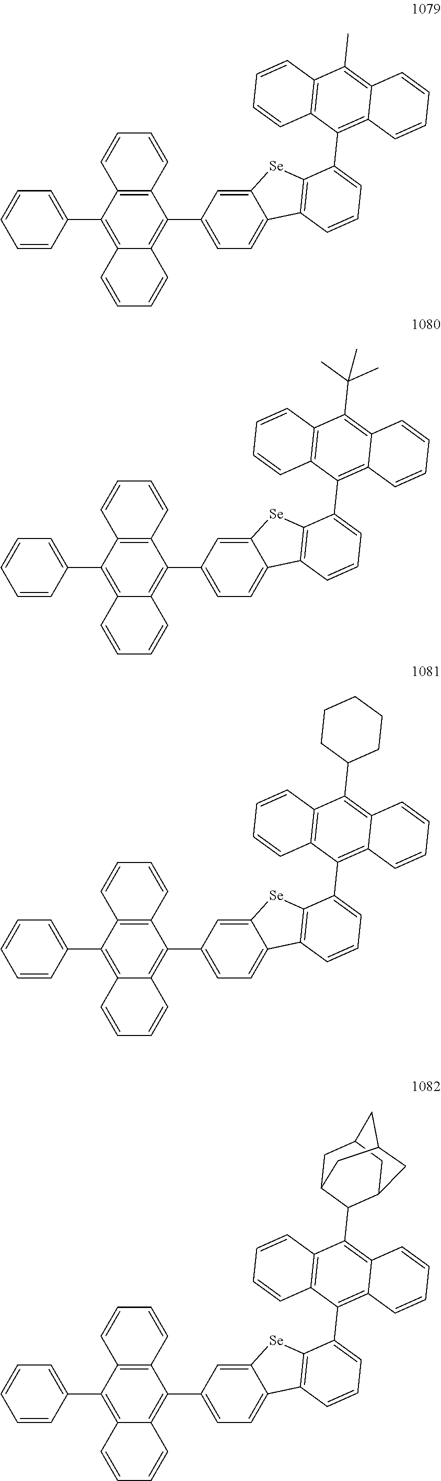 [SMILES: Cc1c2ccccc2c(-c2cccc3c2[se]c2cc(-c4c5ccccc5c(-c5ccccc5)c5ccccc45)ccc23)c2ccccc12 ; CC(C)(C)c1c2ccccc2c(-c2cccc3c2[se]c2cc(-c4c5ccccc5c(-c5ccccc5)c5ccccc45)ccc23)c2ccccc12 ; c1ccc(-c2c3ccccc3c(-c3ccc4c(c3)[se]c3c(-c5c6ccccc6c(C6CCCCC6)c6ccccc56)cccc34)c3ccccc23)cc1 ; c1ccc(-c2c3ccccc3c(-c3ccc4c(c3)[se]c3c(-c5c6ccccc6c(C6C7CC8CC(C7)CC6C8)c6ccccc56)cccc34)c3ccccc23)cc1]

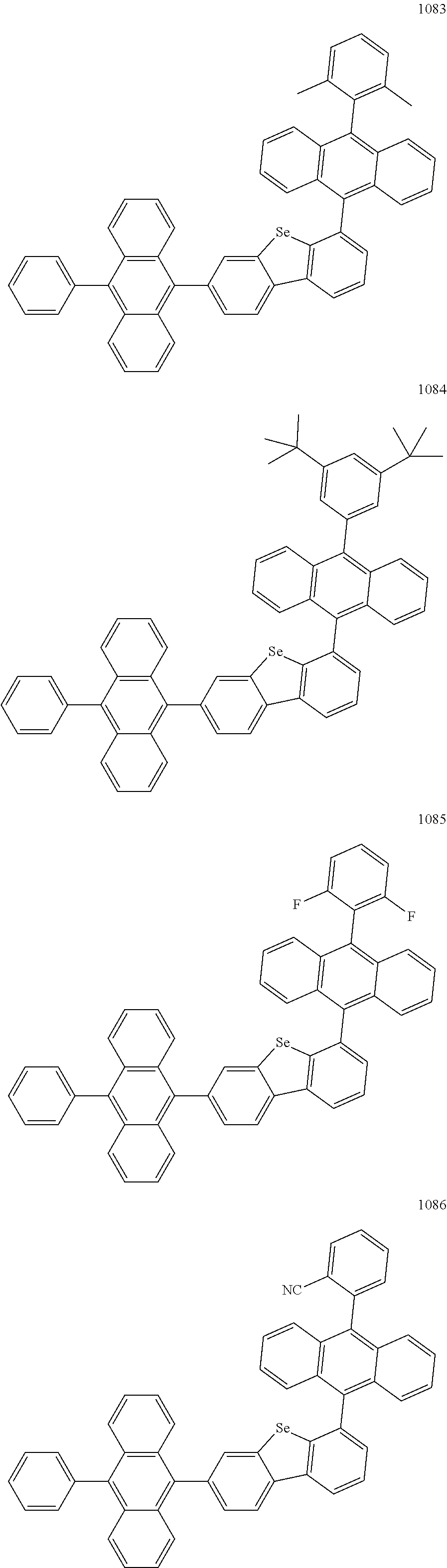 [SMILES: Cc1cccc(C)c1-c1c2ccccc2c(-c2cccc3c2[se]c2cc(-c4c5ccccc5c(-c5ccccc5)c5ccccc45)ccc23)c2ccccc12 ; CC(C)(C)c1cc(-c2c3ccccc3c(-c3cccc4c3[se]c3cc(-c5c6ccccc6c(-c6ccccc6)c6ccccc56)ccc34)c3ccccc23)cc(C(C)(C)C)c1 ; Fc1cccc(F)c1-c1c2ccccc2c(-c2cccc3c2[se]c2cc(-c4c5ccccc5c(-c5ccccc5)c5ccccc45)ccc23)c2ccccc12 ; N#Cc1ccccc1-c1c2ccccc2c(-c2cccc3c2[se]c2cc(-c4c5ccccc5c(-c5ccccc5)c5ccccc45)ccc23)c2ccccc12]

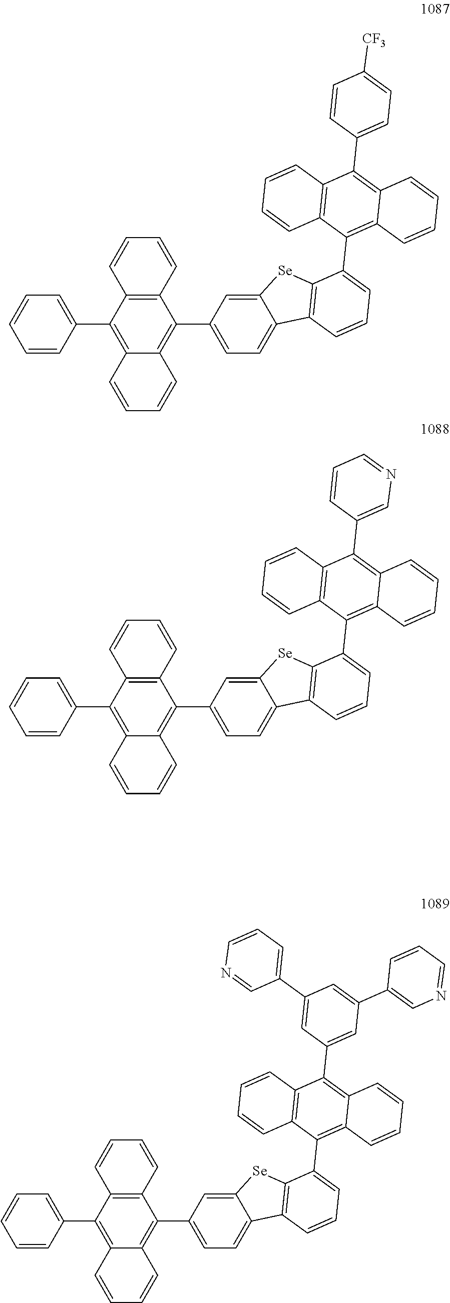 [SMILES: FC(F)(F)c1ccc(-c2c3ccccc3c(-c3cccc4c3[se]c3cc(-c5c6ccccc6c(-c6ccccc6)c6ccccc56)ccc34)c3ccccc23)cc1 ; c1ccc(-c2c3ccccc3c(-c3ccc4c(c3)[se]c3c(-c5c6ccccc6c(-c6cccnc6)c6ccccc56)cccc34)c3ccccc23)cc1 ; c1ccc(-c2c3ccccc3c(-c3ccc4c(c3)[se]c3c(-c5c6ccccc6c(-c6cc(-c7cccnc7)cc(-c7cccnc7)c6)c6ccccc56)cccc34)c3ccccc23)cc1]

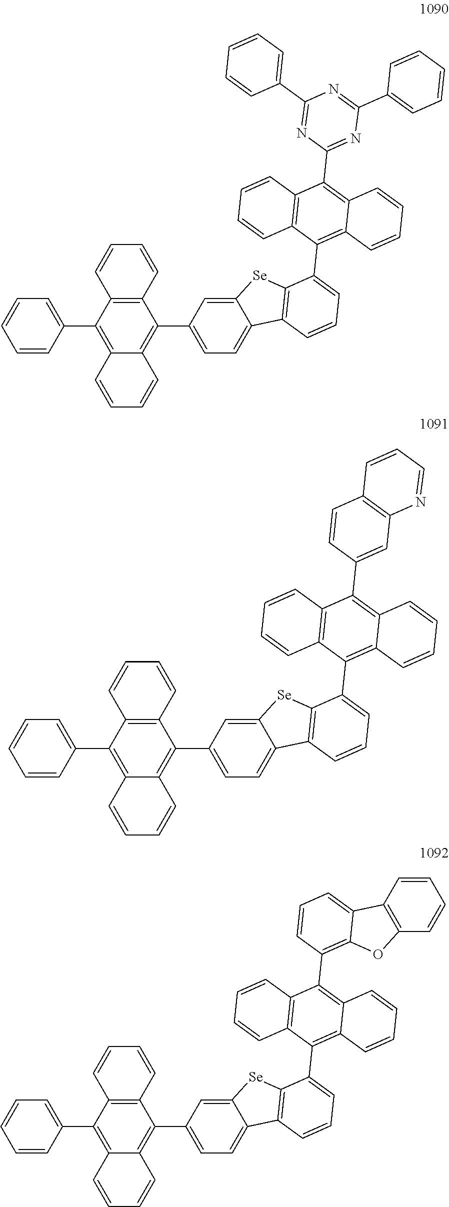 [SMILES: c1ccc(-c2nc(-c3ccccc3)nc(-c3c4ccccc4c(-c4cccc5c4[se]c4cc(-c6c7ccccc7c(-c7ccccc7)c7ccccc67)ccc45)c4ccccc34)n2)cc1 ; c1ccc(-c2c3ccccc3c(-c3ccc4c(c3)[se]c3c(-c5c6ccccc6c(-c6ccc7cccnc7c6)c6ccccc56)cccc34)c3ccccc23)cc1 ; c1ccc(-c2c3ccccc3c(-c3ccc4c(c3)[se]c3c(-c5c6ccccc6c(-c6cccc7c6oc6ccccc67)c6ccccc56)cccc34)c3ccccc23)cc1]

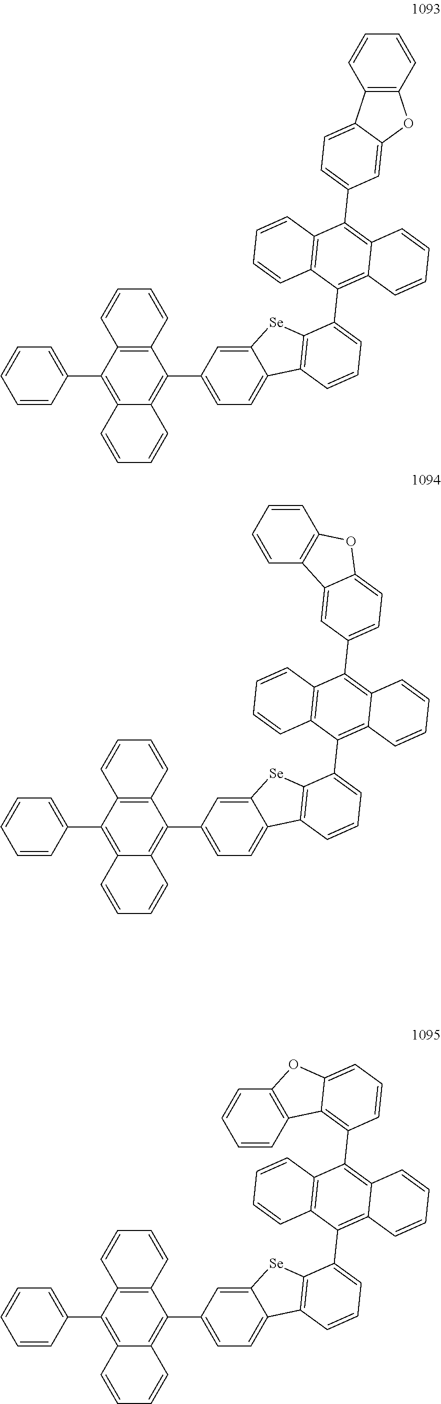 [SMILES: c1ccc(-c2c3ccccc3c(-c3ccc4c(c3)[se]c3c(-c5c6ccccc6c(-c6ccc7c(c6)oc6ccccc67)c6ccccc56)cccc34)c3ccccc23)cc1 ; c1ccc(-c2c3ccccc3c(-c3ccc4c(c3)[se]c3c(-c5c6ccccc6c(-c6ccc7oc8ccccc8c7c6)c6ccccc56)cccc34)c3ccccc23)cc1 ; c1ccc(-c2c3ccccc3c(-c3ccc4c(c3)[se]c3c(-c5c6ccccc6c(-c6cccc7oc8ccccc8c67)c6ccccc56)cccc34)c3ccccc23)cc1]

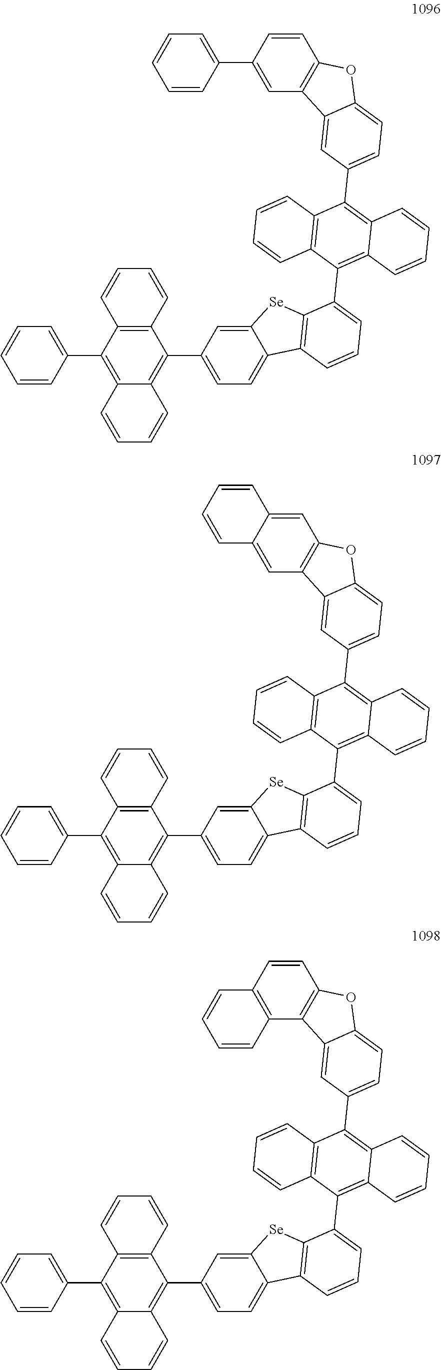 [SMILES: c1ccc(-c2ccc3oc4ccc(-c5c6ccccc6c(-c6cccc7c6[se]c6cc(-c8c9ccccc9c(-c9ccccc9)c9ccccc89)ccc67)c6ccccc56)cc4c3c2)cc1 ; c1ccc(-c2c3ccccc3c(-c3ccc4c(c3)[se]c3c(-c5c6ccccc6c(-c6ccc7oc8cc9ccccc9cc8c7c6)c6ccccc56)cccc34)c3ccccc23)cc1 ; c1ccc(-c2c3ccccc3c(-c3ccc4c(c3)[se]c3c(-c5c6ccccc6c(-c6ccc7oc8ccc9ccccc9c8c7c6)c6ccccc56)cccc34)c3ccccc23)cc1]

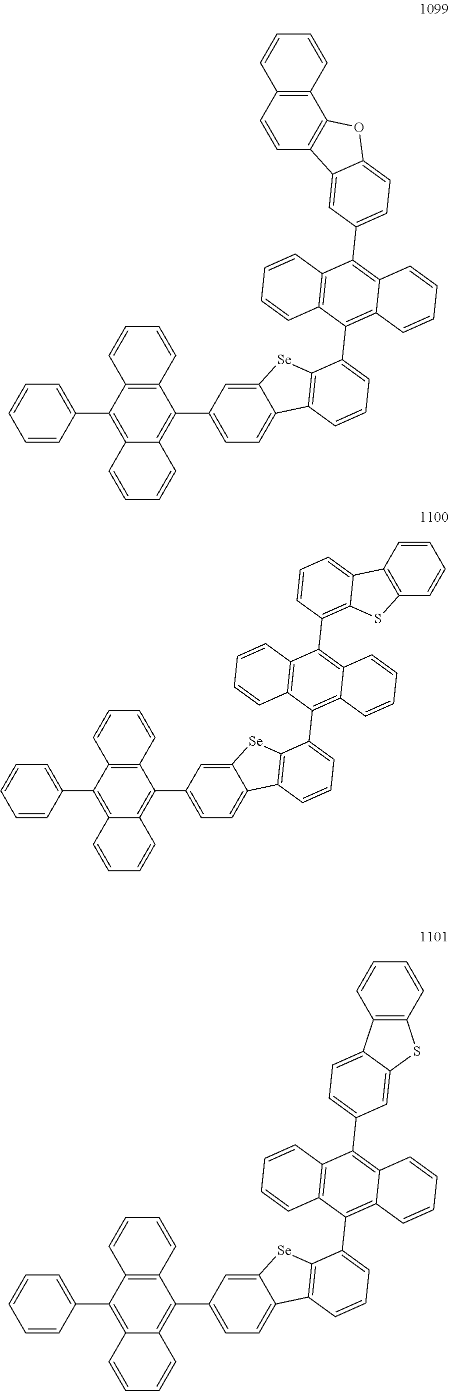 [SMILES: c1ccc(-c2c3ccccc3c(-c3ccc4c(c3)[se]c3c(-c5c6ccccc6c(-c6ccc7oc8c9ccccc9ccc8c7c6)c6ccccc56)cccc34)c3ccccc23)cc1 ; c1ccc(-c2c3ccccc3c(-c3ccc4c(c3)[se]c3c(-c5c6ccccc6c(-c6cccc7c6sc6ccccc67)c6ccccc56)cccc34)c3ccccc23)cc1 ; c1ccc(-c2c3ccccc3c(-c3ccc4c(c3)[se]c3c(-c5c6ccccc6c(-c6ccc7c(c6)sc6ccccc67)c6ccccc56)cccc34)c3ccccc23)cc1]

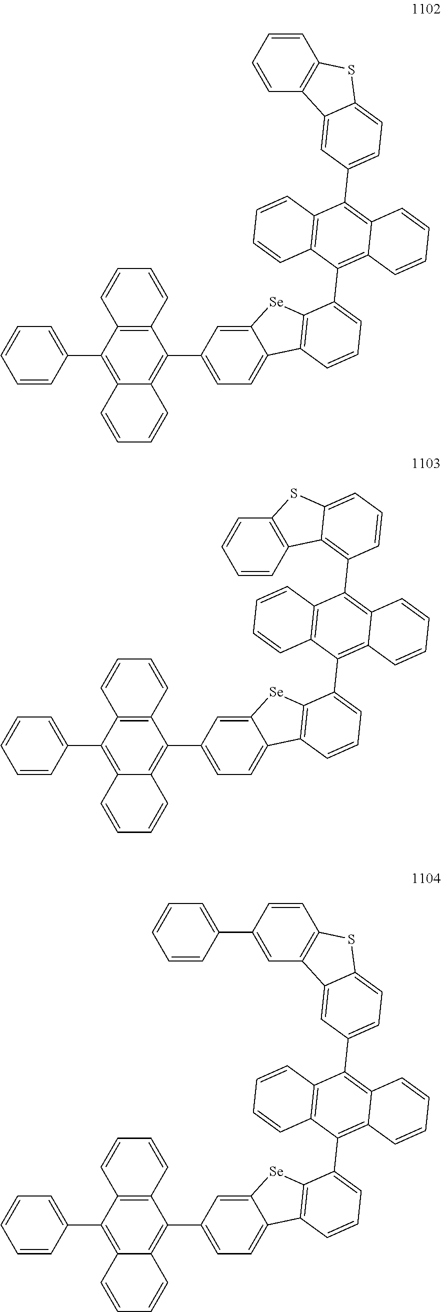 [SMILES: c1ccc(-c2c3ccccc3c(-c3ccc4c(c3)[se]c3c(-c5c6ccccc6c(-c6ccc7sc8ccccc8c7c6)c6ccccc56)cccc34)c3ccccc23)cc1 ; c1ccc(-c2c3ccccc3c(-c3ccc4c(c3)[se]c3c(-c5c6ccccc6c(-c6cccc7sc8ccccc8c67)c6ccccc56)cccc34)c3ccccc23)cc1 ; c1ccc(-c2ccc3sc4ccc(-c5c6ccccc6c(-c6cccc7c6[se]c6cc(-c8c9ccccc9c(-c9ccccc9)c9ccccc89)ccc67)c6ccccc56)cc4c3c2)cc1]

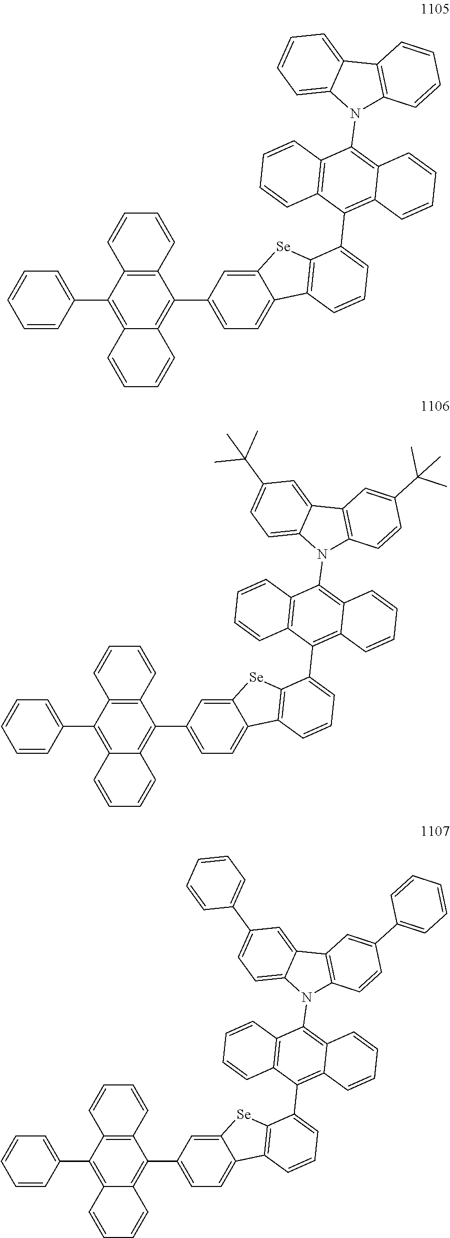 [SMILES: c1ccc(-c2c3ccccc3c(-c3ccc4c(c3)[se]c3c(-c5c6ccccc6c(-n6c7ccccc7c7ccccc76)c6ccccc56)cccc34)c3ccccc23)cc1 ; CC(C)(C)c1ccc2c(c1)c1cc(C(C)(C)C)ccc1n2-c1c2ccccc2c(-c2cccc3c2[se]c2cc(-c4c5ccccc5c(-c5ccccc5)c5ccccc45)ccc23)c2ccccc12 ; c1ccc(-c2ccc3c(c2)c2cc(-c4ccccc4)ccc2n3-c2c3ccccc3c(-c3cccc4c3[se]c3cc(-c5c6ccccc6c(-c6ccccc6)c6ccccc56)ccc34)c3ccccc23)cc1]

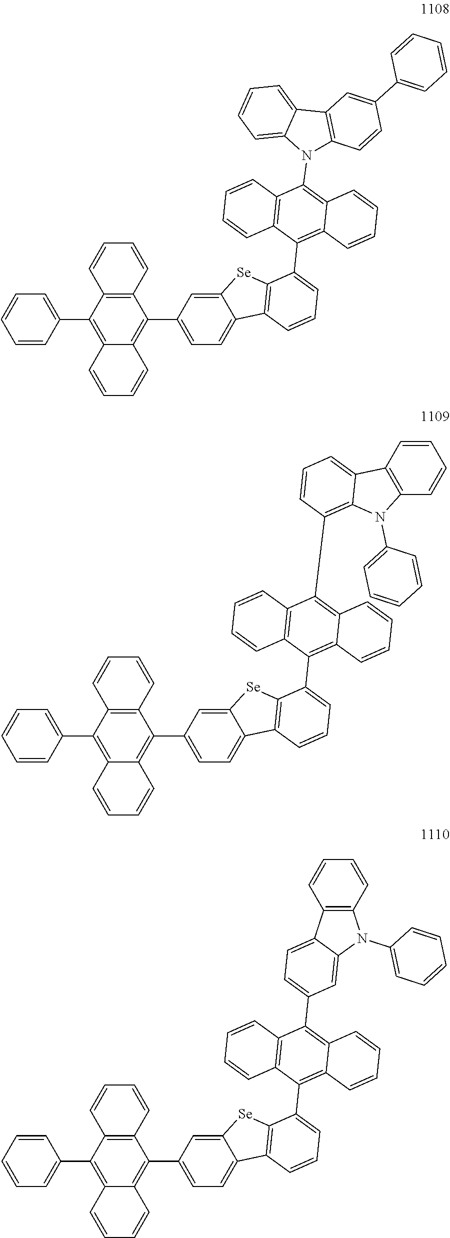 [SMILES: c1ccc(-c2ccc3c(c2)c2ccccc2n3-c2c3ccccc3c(-c3cccc4c3[se]c3cc(-c5c6ccccc6c(-c6ccccc6)c6ccccc56)ccc34)c3ccccc23)cc1 ; c1ccc(-c2c3ccccc3c(-c3ccc4c(c3)[se]c3c(-c5c6ccccc6c(-c6cccc7c8ccccc8n(-c8ccccc8)c67)c6ccccc56)cccc34)c3ccccc23)cc1 ; c1ccc(-c2c3ccccc3c(-c3ccc4c(c3)[se]c3c(-c5c6ccccc6c(-c6ccc7c8ccccc8n(-c8ccccc8)c7c6)c6ccccc56)cccc34)c3ccccc23)cc1]

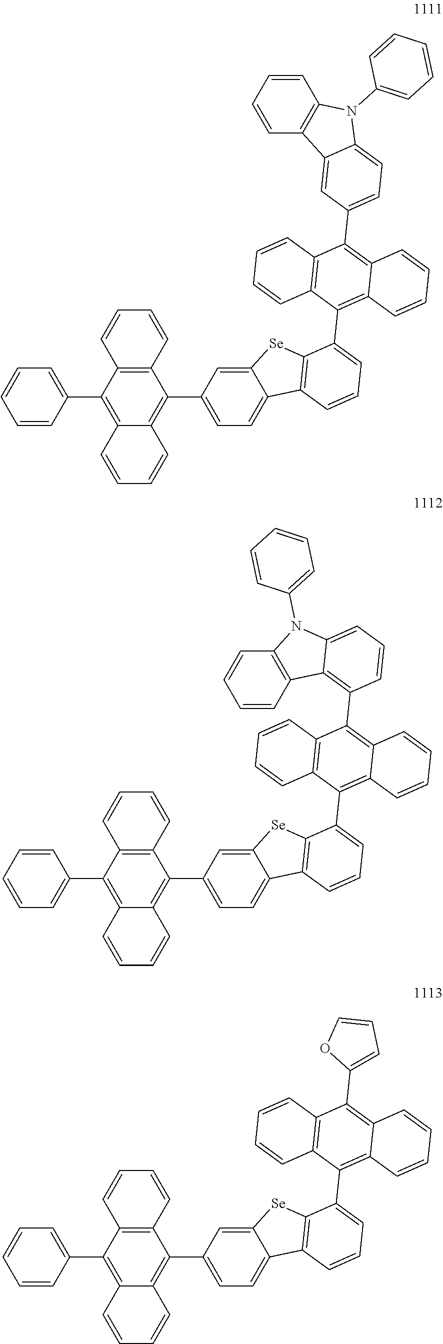

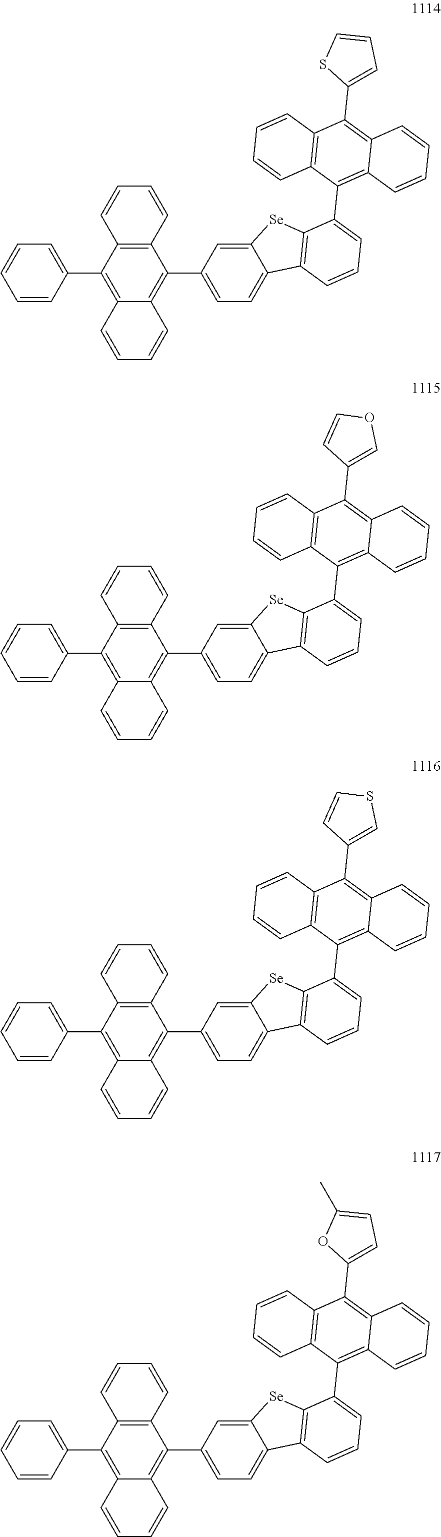

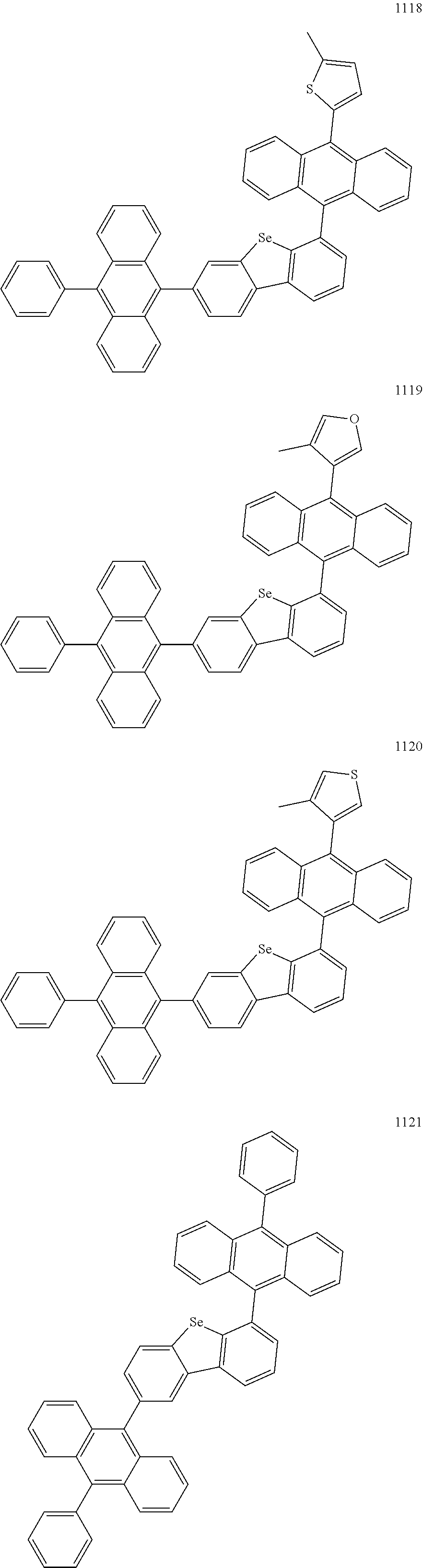

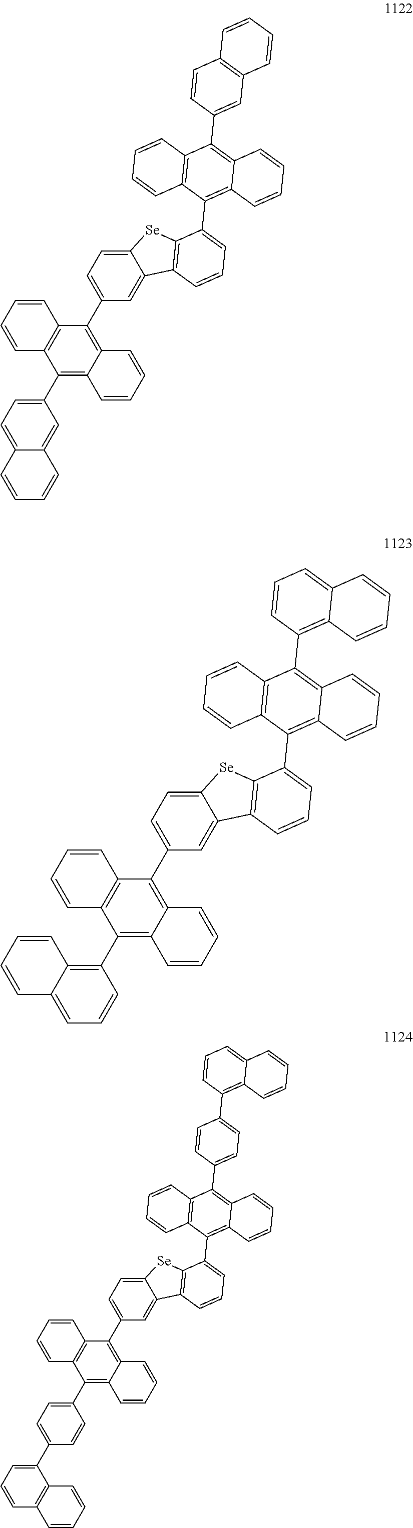 [SMILES: c1ccc2cc(-c3c4ccccc4c(-c4ccc5[se]c6c(-c7c8ccccc8c(-c8ccc9ccccc9c8)c8ccccc78)cccc6c5c4)c4ccccc34)ccc2c1 ; c1ccc2c(-c3c4ccccc4c(-c4ccc5[se]c6c(-c7c8ccccc8c(-c8cccc9ccccc89)c8ccccc78)cccc6c5c4)c4ccccc34)cccc2c1 ; c1ccc2c(-c3ccc(-c4c5ccccc5c(-c5ccc6[se]c7c(-c8c9ccccc9c(-c9ccc(-c%10cccc%11ccccc%10%11)cc9)c9ccccc89)cccc7c6c5)c5ccccc45)cc3)cccc2c1]

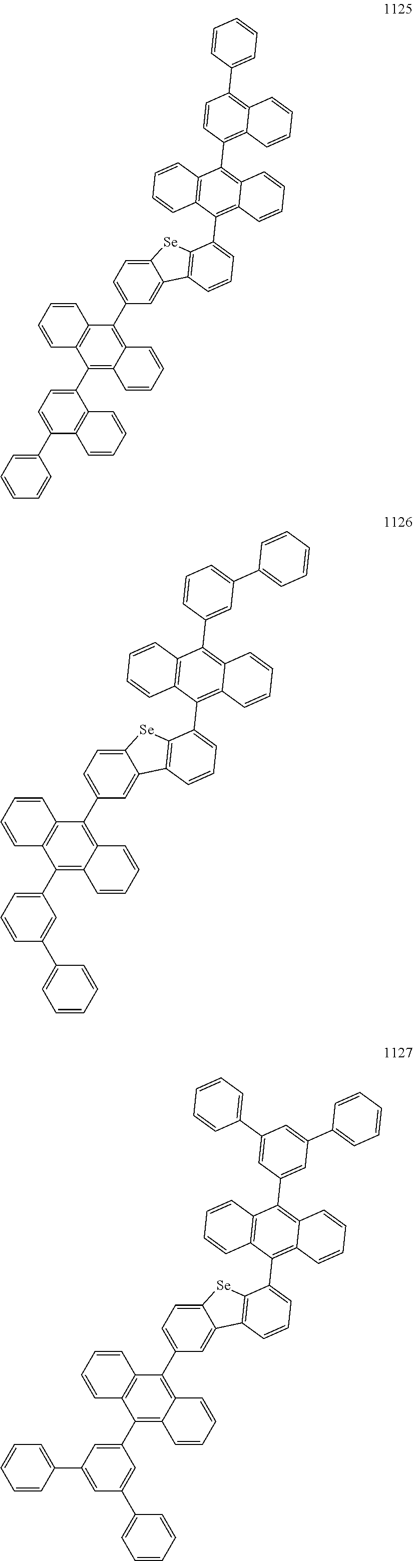 [SMILES: c1ccc(-c2ccc(-c3c4ccccc4c(-c4ccc5[se]c6c(-c7c8ccccc8c(-c8ccc(-c9ccccc9)c9ccccc89)c8ccccc78)cccc6c5c4)c4ccccc34)c3ccccc23)cc1 ; c1ccc(-c2cccc(-c3c4ccccc4c(-c4ccc5[se]c6c(-c7c8ccccc8c(-c8cccc(-c9ccccc9)c8)c8ccccc78)cccc6c5c4)c4ccccc34)c2)cc1 ; c1ccc(-c2cc(-c3ccccc3)cc(-c3c4ccccc4c(-c4ccc5[se]c6c(-c7c8ccccc8c(-c8cc(-c9ccccc9)cc(-c9ccccc9)c8)c8ccccc78)cccc6c5c4)c4ccccc34)c2)cc1]

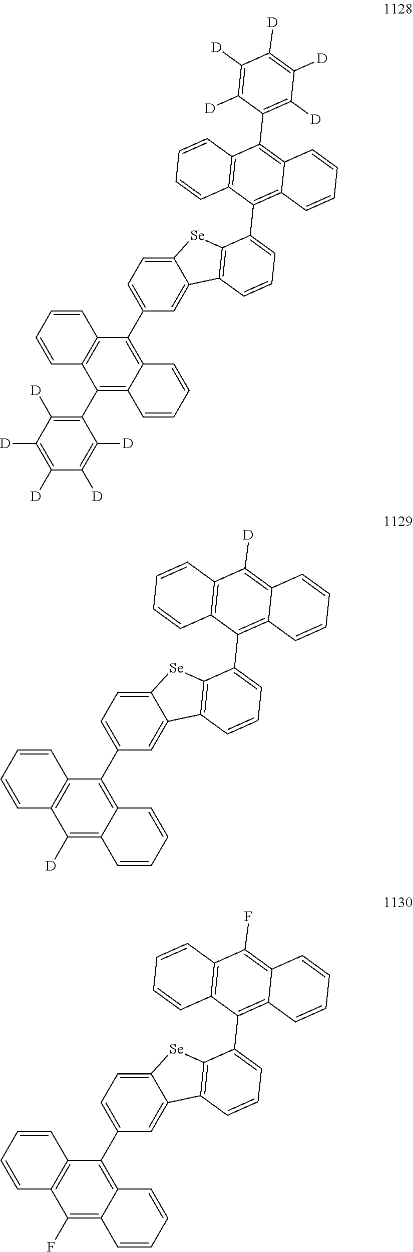 [SMILES: [2H]c1c([2H])c([2H])c(-c2c3ccccc3c(-c3ccc4[se]c5c(-c6c7ccccc7c(-c7c([2H])c([2H])c([2H])c([2H])c7[2H])c7ccccc67)cccc5c4c3)c3ccccc23)c([2H])c1[2H] ; [2H]c1c2ccccc2c(-c2ccc3[se]c4c(-c5c6ccccc6c([2H])c6ccccc56)cccc4c3c2)c2ccccc12 ; Fc1c2ccccc2c(-c2ccc3[se]c4c(-c5c6ccccc6c(F)c6ccccc56)cccc4c3c2)c2ccccc12]

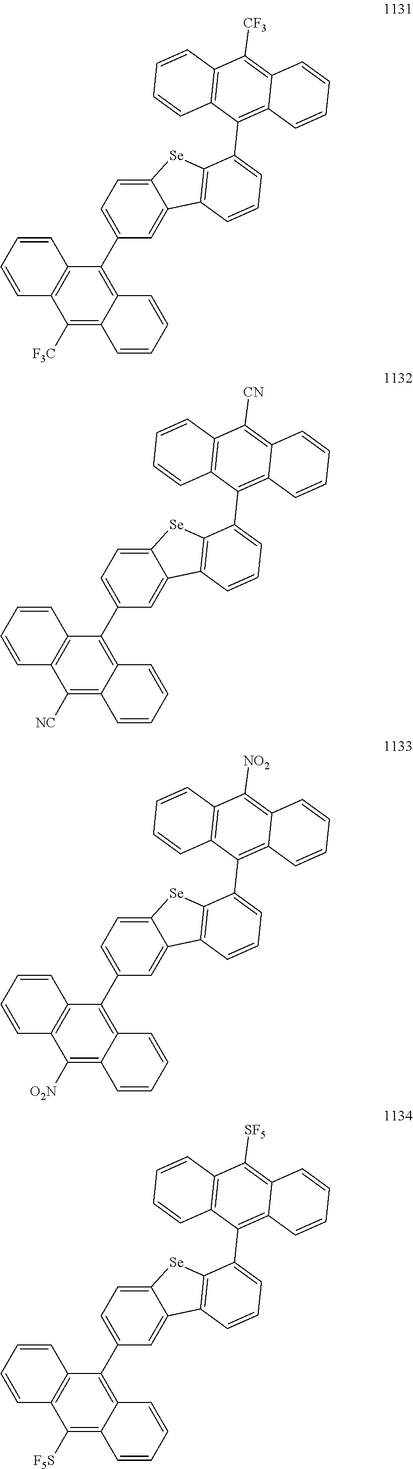

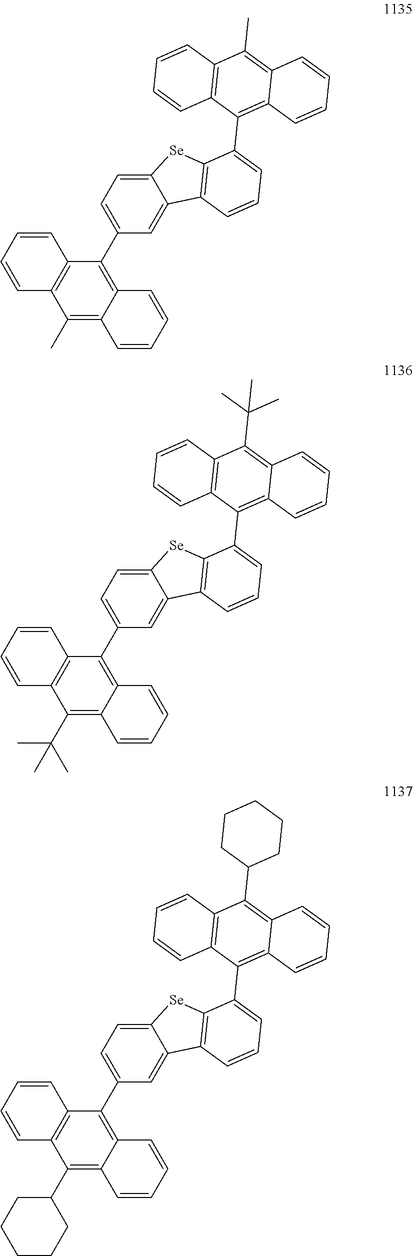 [SMILES: Cc1c2ccccc2c(-c2ccc3[se]c4c(-c5c6ccccc6c(C)c6ccccc56)cccc4c3c2)c2ccccc12 ; CC(C)(C)c1c2ccccc2c(-c2ccc3[se]c4c(-c5c6ccccc6c(C(C)(C)C)c6ccccc56)cccc4c3c2)c2ccccc12 ; c1ccc2c(C3CCCCC3)c3ccccc3c(-c3ccc4[se]c5c(-c6c7ccccc7c(C7CCCCC7)c7ccccc67)cccc5c4c3)c2c1]

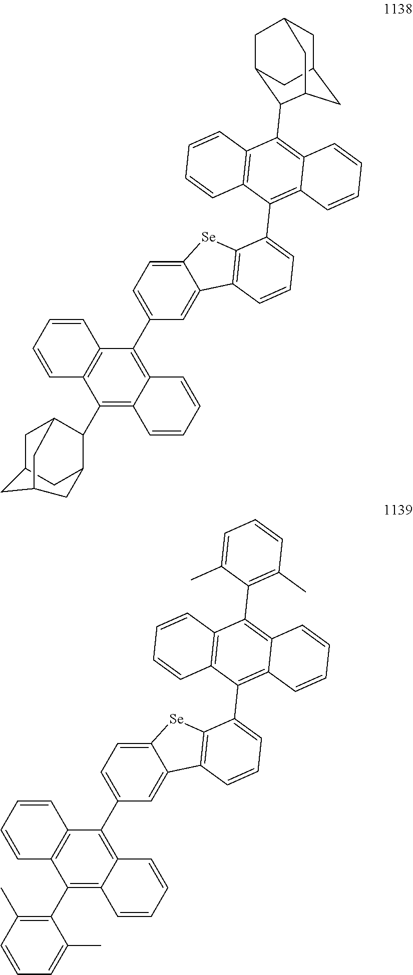 [SMILES: c1ccc2c(C3C4CC5CC(C4)CC3C5)c3ccccc3c(-c3ccc4[se]c5c(-c6c7ccccc7c(C7C8CC9CC(C8)CC7C9)c7ccccc67)cccc5c4c3)c2c1 ; Cc1cccc(C)c1-c1c2ccccc2c(-c2ccc3[se]c4c(-c5c6ccccc6c(-c6c(C)cccc6C)c6ccccc56)cccc4c3c2)c2ccccc12]

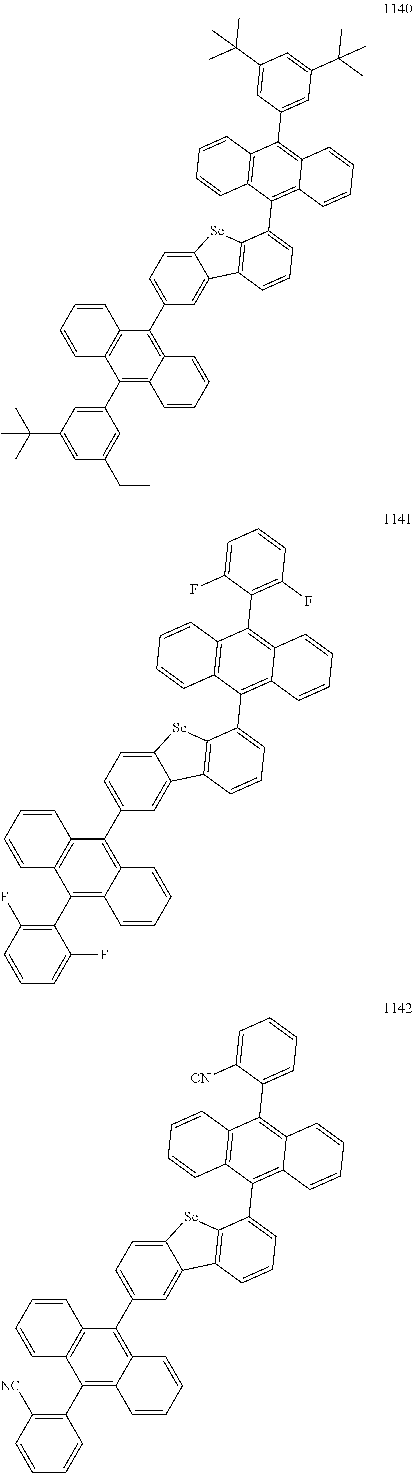

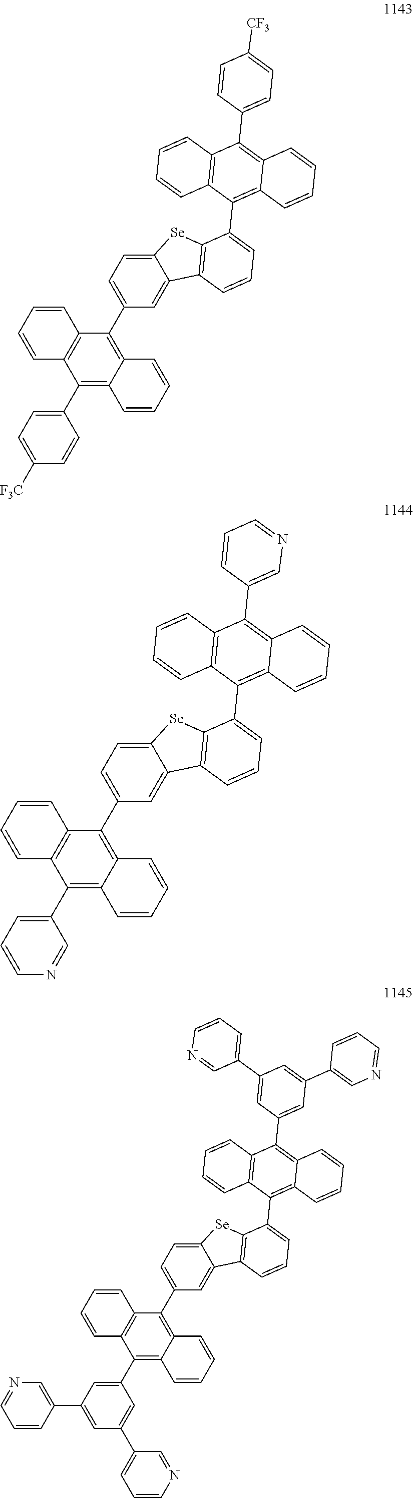 [SMILES: FC(F)(F)c1ccc(-c2c3ccccc3c(-c3ccc4[se]c5c(-c6c7ccccc7c(-c7ccc(C(F)(F)F)cc7)c7ccccc67)cccc5c4c3)c3ccccc23)cc1 ; c1cncc(-c2c3ccccc3c(-c3ccc4[se]c5c(-c6c7ccccc7c(-c7cccnc7)c7ccccc67)cccc5c4c3)c3ccccc23)c1 ; c1cncc(-c2cc(-c3cccnc3)cc(-c3c4ccccc4c(-c4ccc5[se]c6c(-c7c8ccccc8c(-c8cc(-c9cccnc9)cc(-c9cccnc9)c8)c8ccccc78)cccc6c5c4)c4ccccc34)c2)c1]

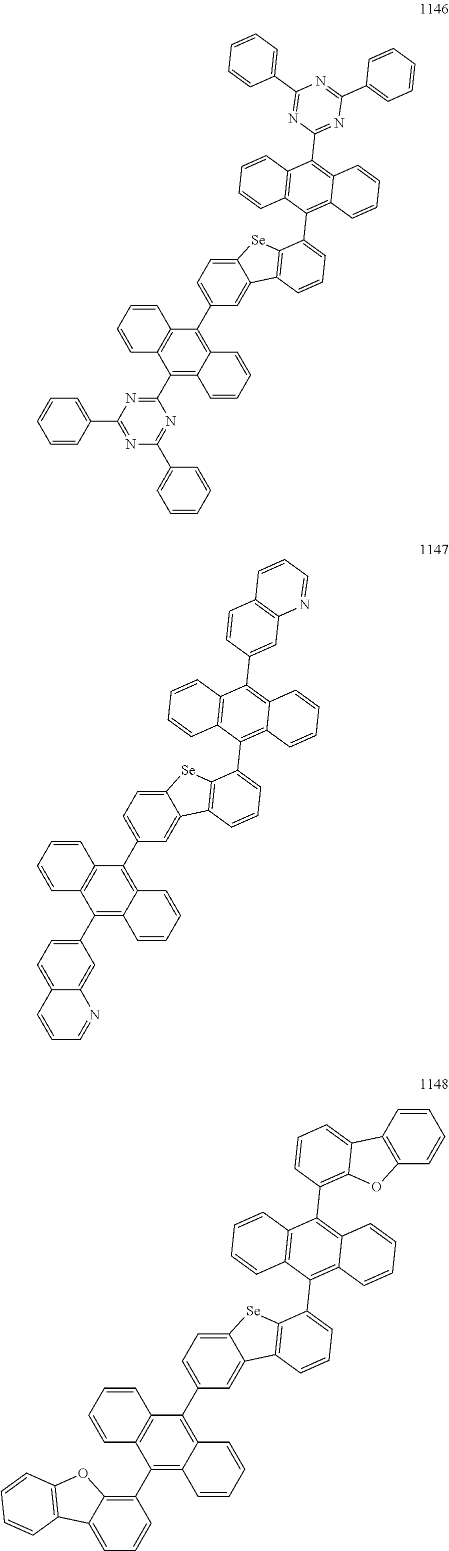 [SMILES: c1ccc(-c2nc(-c3ccccc3)nc(-c3c4ccccc4c(-c4ccc5[se]c6c(-c7c8ccccc8c(-c8nc(-c9ccccc9)nc(-c9ccccc9)n8)c8ccccc78)cccc6c5c4)c4ccccc34)n2)cc1 ; c1cnc2cc(-c3c4ccccc4c(-c4ccc5[se]c6c(-c7c8ccccc8c(-c8ccc9cccnc9c8)c8ccccc78)cccc6c5c4)c4ccccc34)ccc2c1 ; c1ccc2c(c1)oc1c(-c3c4ccccc4c(-c4ccc5[se]c6c(-c7c8ccccc8c(-c8cccc9c8oc8ccccc89)c8ccccc78)cccc6c5c4)c4ccccc34)cccc12]

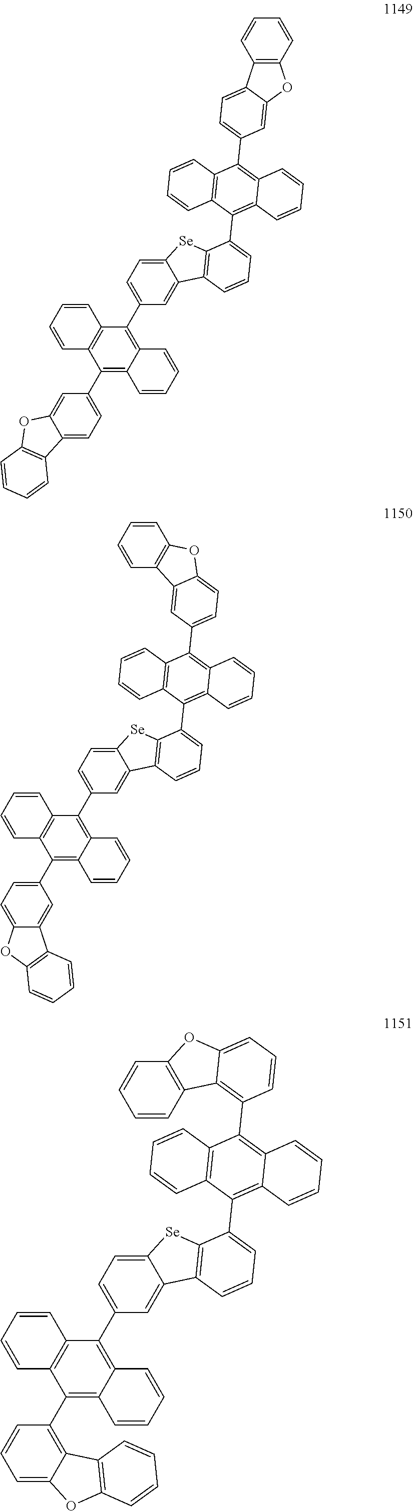 [SMILES: c1ccc2c(c1)oc1cc(-c3c4ccccc4c(-c4ccc5[se]c6c(-c7c8ccccc8c(-c8ccc9c(c8)oc8ccccc89)c8ccccc78)cccc6c5c4)c4ccccc34)ccc12 ; c1ccc2c(c1)oc1ccc(-c3c4ccccc4c(-c4ccc5[se]c6c(-c7c8ccccc8c(-c8ccc9oc%10ccccc%10c9c8)c8ccccc78)cccc6c5c4)c4ccccc34)cc12 ; c1ccc2c(c1)oc1cccc(-c3c4ccccc4c(-c4ccc5[se]c6c(-c7c8ccccc8c(-c8cccc9oc%10ccccc%10c89)c8ccccc78)cccc6c5c4)c4ccccc34)c12]

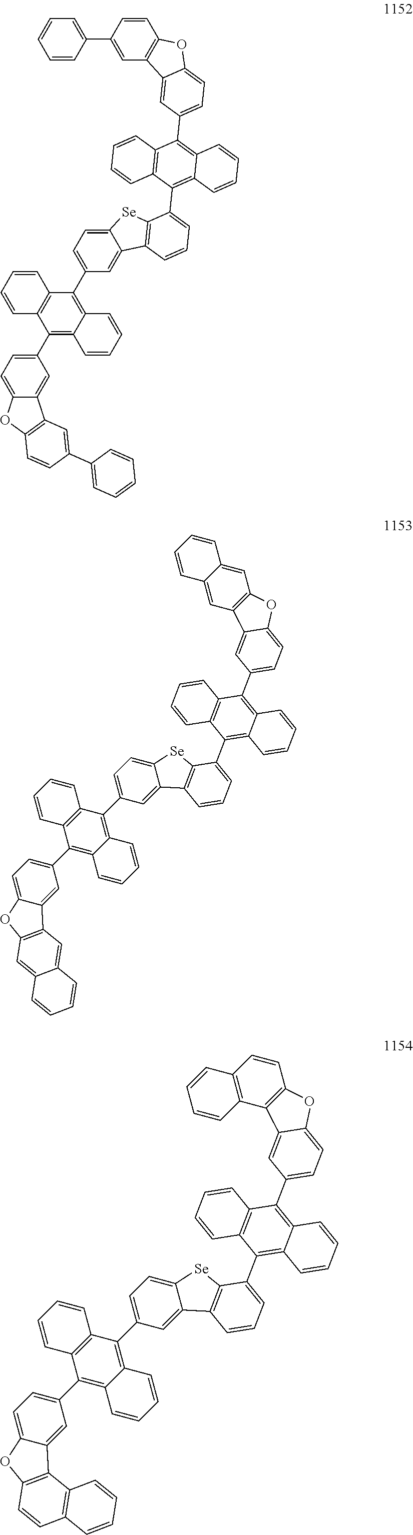 [SMILES: c1ccc(-c2ccc3oc4ccc(-c5c6ccccc6c(-c6ccc7[se]c8c(-c9c%10ccccc%10c(-c%10ccc%11oc%12ccc(-c%13ccccc%13)cc%12c%11c%10)c%10ccccc9%10)cccc8c7c6)c6ccccc56)cc4c3c2)cc1 ; c1ccc2cc3c(cc2c1)oc1ccc(-c2c4ccccc4c(-c4ccc5[se]c6c(-c7c8ccccc8c(-c8ccc9oc%10cc%11ccccc%11cc%10c9c8)c8ccccc78)cccc6c5c4)c4ccccc24)cc13 ; c1ccc2c(c1)ccc1oc3ccc(-c4c5ccccc5c(-c5ccc6[se]c7c(-c8c9ccccc9c(-c9ccc%10oc%11ccc%12ccccc%12c%11c%10c9)c9ccccc89)cccc7c6c5)c5ccccc45)cc3c12]

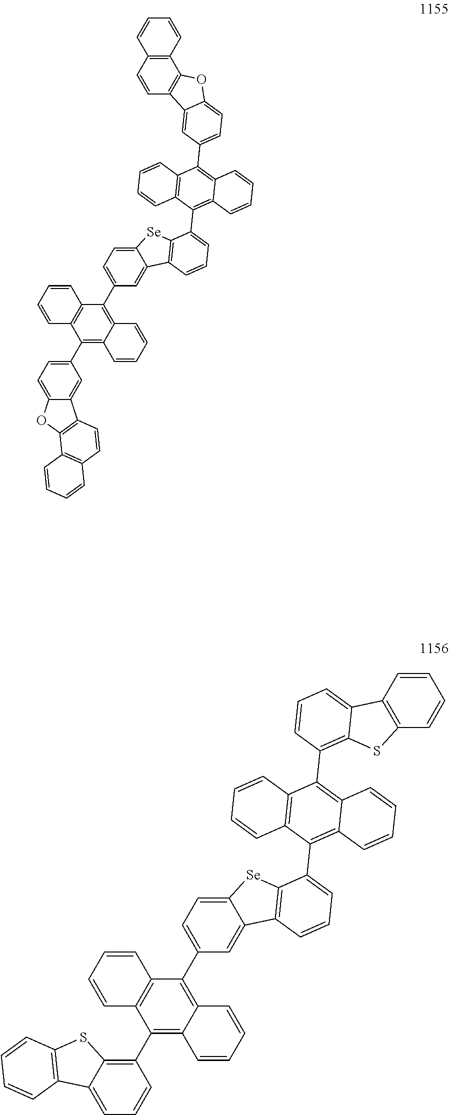 [SMILES: c1ccc2c(c1)ccc1c3cc(-c4c5ccccc5c(-c5ccc6[se]c7c(-c8c9ccccc9c(-c9ccc%10oc%11c%12ccccc%12ccc%11c%10c9)c9ccccc89)cccc7c6c5)c5ccccc45)ccc3oc21 ; c1ccc2c(c1)sc1c(-c3c4ccccc4c(-c4ccc5[se]c6c(-c7c8ccccc8c(-c8cccc9c8sc8ccccc89)c8ccccc78)cccc6c5c4)c4ccccc34)cccc12]

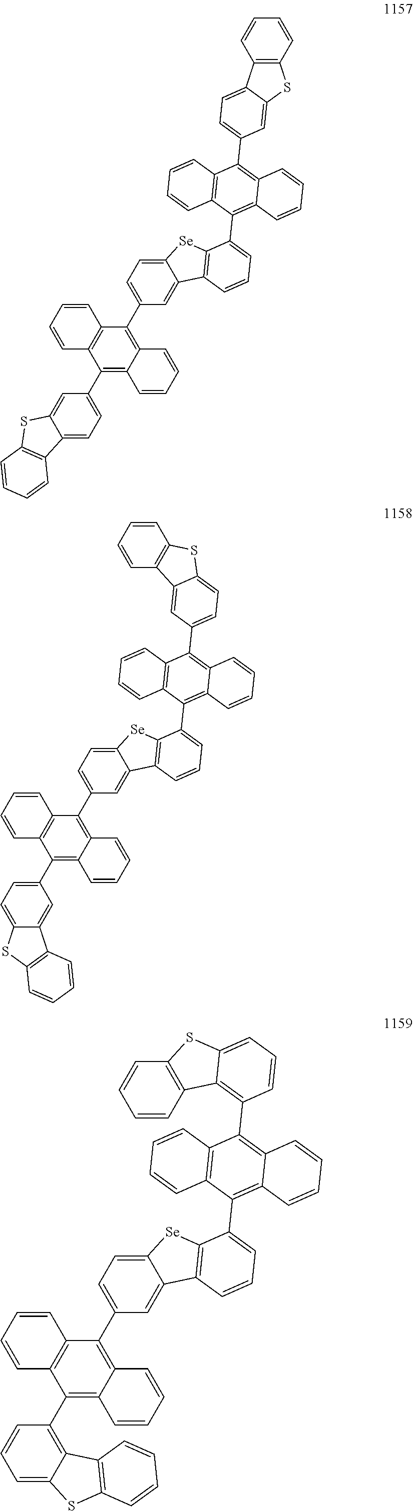 [SMILES: c1ccc2c(c1)sc1cc(-c3c4ccccc4c(-c4ccc5[se]c6c(-c7c8ccccc8c(-c8ccc9c(c8)sc8ccccc89)c8ccccc78)cccc6c5c4)c4ccccc34)ccc12 ; c1ccc2c(c1)sc1ccc(-c3c4ccccc4c(-c4ccc5[se]c6c(-c7c8ccccc8c(-c8ccc9sc%10ccccc%10c9c8)c8ccccc78)cccc6c5c4)c4ccccc34)cc12 ; c1ccc2c(c1)sc1cccc(-c3c4ccccc4c(-c4ccc5[se]c6c(-c7c8ccccc8c(-c8cccc9sc%10ccccc%10c89)c8ccccc78)cccc6c5c4)c4ccccc34)c12]

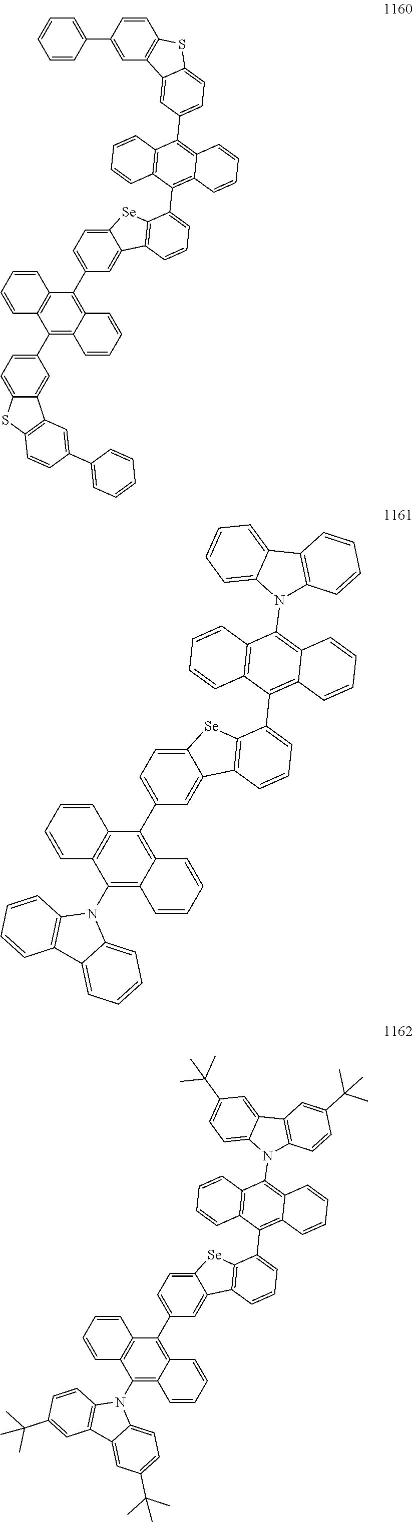 [SMILES: c1ccc(-c2ccc3sc4ccc(-c5c6ccccc6c(-c6ccc7[se]c8c(-c9c%10ccccc%10c(-c%10ccc%11sc%12ccc(-c%13ccccc%13)cc%12c%11c%10)c%10ccccc9%10)cccc8c7c6)c6ccccc56)cc4c3c2)cc1 ; c1ccc2c(-n3c4ccccc4c4ccccc43)c3ccccc3c(-c3ccc4[se]c5c(-c6c7ccccc7c(-n7c8ccccc8c8ccccc87)c7ccccc67)cccc5c4c3)c2c1 ; CC(C)(C)c1ccc2c(c1)c1cc(C(C)(C)C)ccc1n2-c1c2ccccc2c(-c2ccc3[se]c4c(-c5c6ccccc6c(-n6c7ccc(C(C)(C)C)cc7c7cc(C(C)(C)C)ccc76)c6ccccc56)cccc4c3c2)c2ccccc12]

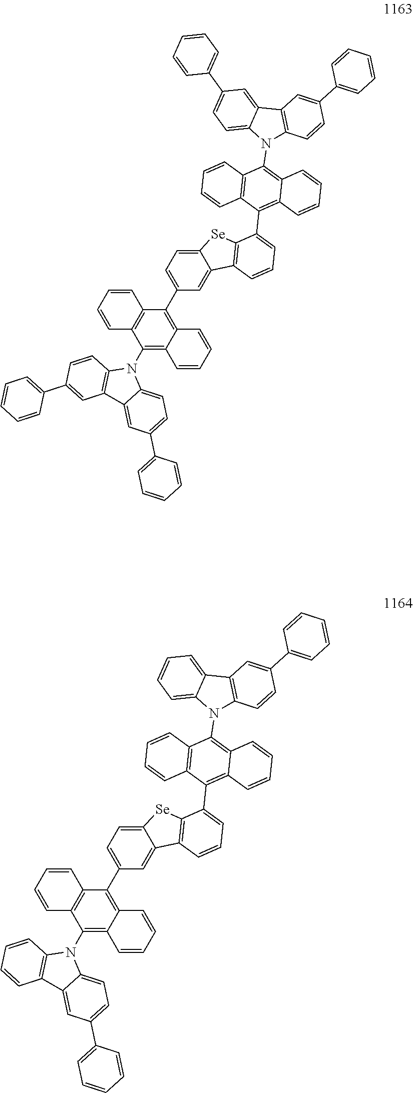 [SMILES: c1ccc(-c2ccc3c(c2)c2cc(-c4ccccc4)ccc2n3-c2c3ccccc3c(-c3ccc4[se]c5c(-c6c7ccccc7c(-n7c8ccc(-c9ccccc9)cc8c8cc(-c9ccccc9)ccc87)c7ccccc67)cccc5c4c3)c3ccccc23)cc1 ; c1ccc(-c2ccc3c(c2)c2ccccc2n3-c2c3ccccc3c(-c3ccc4[se]c5c(-c6c7ccccc7c(-n7c8ccccc8c8cc(-c9ccccc9)ccc87)c7ccccc67)cccc5c4c3)c3ccccc23)cc1]

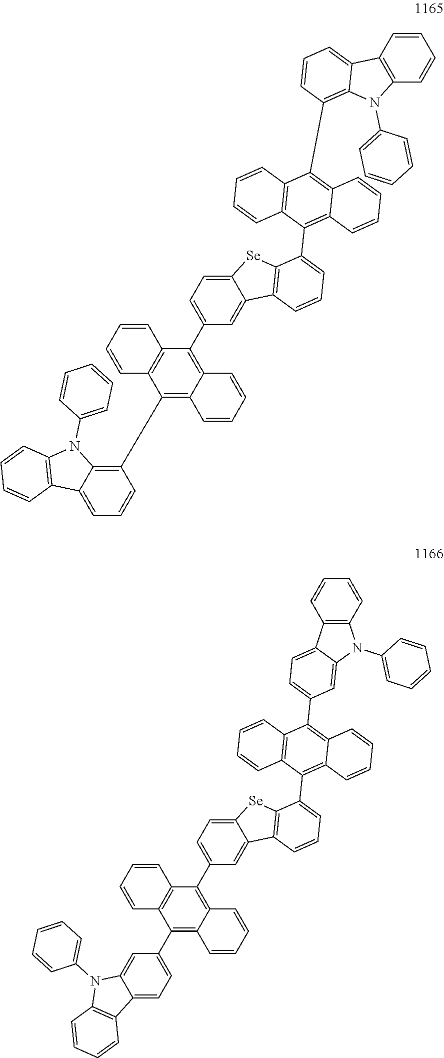 [SMILES: c1ccc(-n2c3ccccc3c3cccc(-c4c5ccccc5c(-c5ccc6[se]c7c(-c8c9ccccc9c(-c9cccc%10c%11ccccc%11n(-c%11ccccc%11)c9%10)c9ccccc89)cccc7c6c5)c5ccccc45)c32)cc1 ; c1ccc(-n2c3ccccc3c3ccc(-c4c5ccccc5c(-c5ccc6[se]c7c(-c8c9ccccc9c(-c9ccc%10c%11ccccc%11n(-c%11ccccc%11)c%10c9)c9ccccc89)cccc7c6c5)c5ccccc45)cc32)cc1]

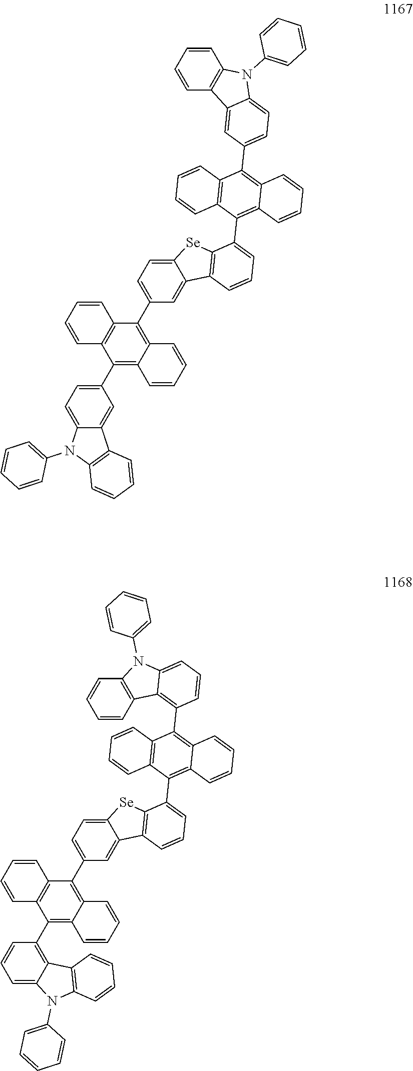 [SMILES: c1ccc(-n2c3ccccc3c3cc(-c4c5ccccc5c(-c5ccc6[se]c7c(-c8c9ccccc9c(-c9ccc%10c(c9)c9ccccc9n%10-c9ccccc9)c9ccccc89)cccc7c6c5)c5ccccc45)ccc32)cc1 ; c1ccc(-n2c3ccccc3c3c(-c4c5ccccc5c(-c5ccc6[se]c7c(-c8c9ccccc9c(-c9cccc%10c9c9ccccc9n%10-c9ccccc9)c9ccccc89)cccc7c6c5)c5ccccc45)cccc32)cc1]

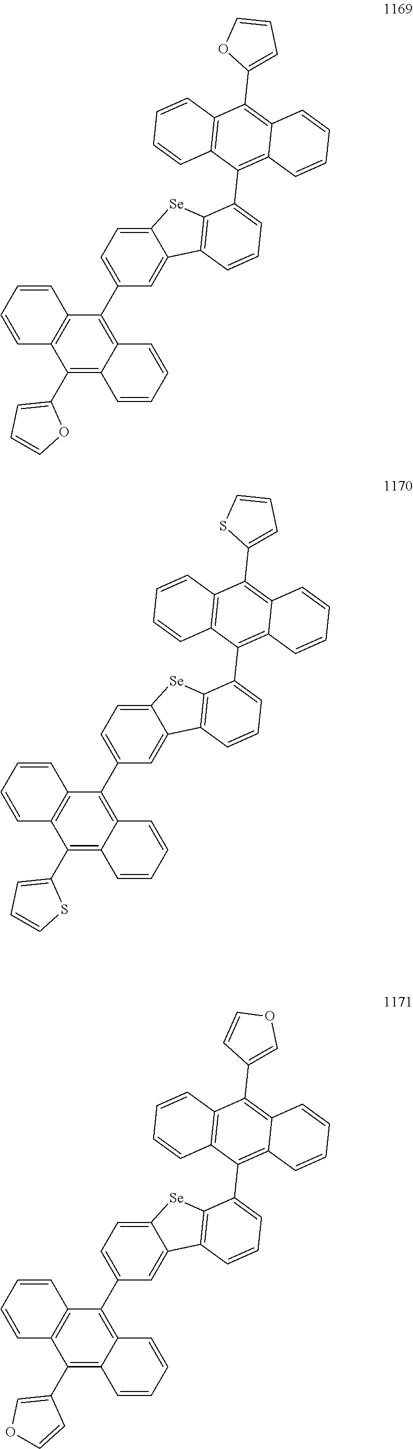 [SMILES: c1coc(-c2c3ccccc3c(-c3ccc4[se]c5c(-c6c7ccccc7c(-c7ccco7)c7ccccc67)cccc5c4c3)c3ccccc23)c1 ; c1csc(-c2c3ccccc3c(-c3ccc4[se]c5c(-c6c7ccccc7c(-c7cccs7)c7ccccc67)cccc5c4c3)c3ccccc23)c1 ; c1ccc2c(-c3ccc4[se]c5c(-c6c7ccccc7c(-c7ccoc7)c7ccccc67)cccc5c4c3)c3ccccc3c(-c3ccoc3)c2c1]

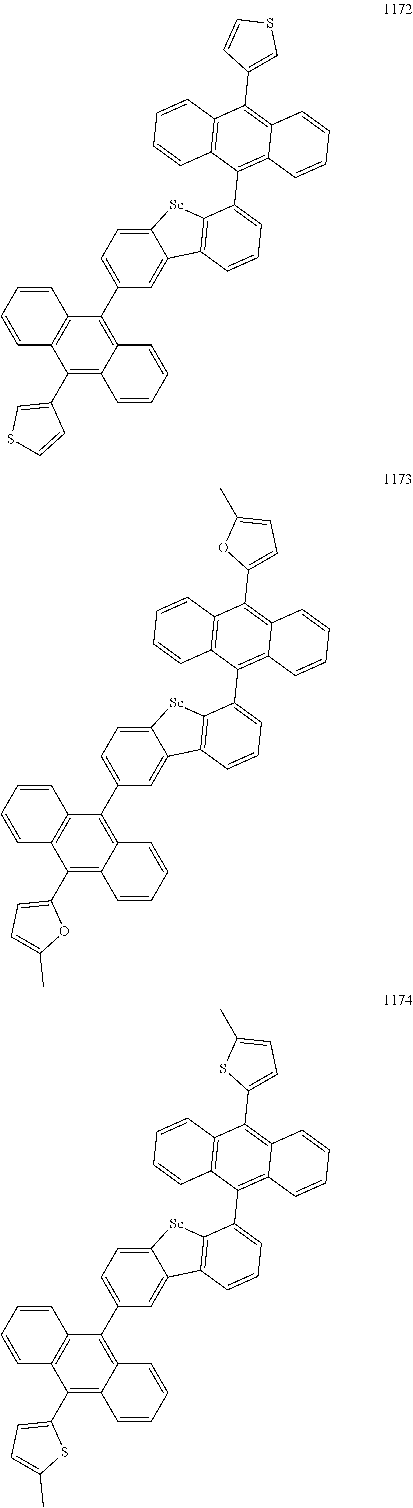 [SMILES: c1ccc2c(-c3ccc4[se]c5c(-c6c7ccccc7c(-c7ccsc7)c7ccccc67)cccc5c4c3)c3ccccc3c(-c3ccsc3)c2c1 ; Cc1ccc(-c2c3ccccc3c(-c3ccc4[se]c5c(-c6c7ccccc7c(-c7ccc(C)o7)c7ccccc67)cccc5c4c3)c3ccccc23)o1 ; Cc1ccc(-c2c3ccccc3c(-c3ccc4[se]c5c(-c6c7ccccc7c(-c7ccc(C)s7)c7ccccc67)cccc5c4c3)c3ccccc23)s1]

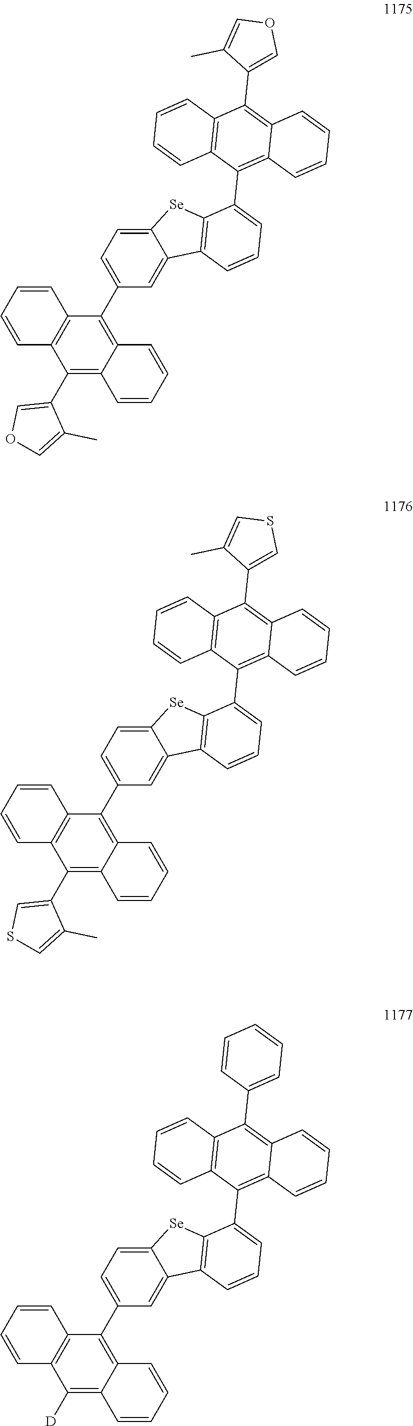 [SMILES: Cc1cocc1-c1c2ccccc2c(-c2ccc3[se]c4c(-c5c6ccccc6c(-c6cocc6C)c6ccccc56)cccc4c3c2)c2ccccc12 ; Cc1cscc1-c1c2ccccc2c(-c2ccc3[se]c4c(-c5c6ccccc6c(-c6cscc6C)c6ccccc56)cccc4c3c2)c2ccccc12 ; [2H]c1c2ccccc2c(-c2ccc3[se]c4c(-c5c6ccccc6c(-c6ccccc6)c6ccccc56)cccc4c3c2)c2ccccc12]

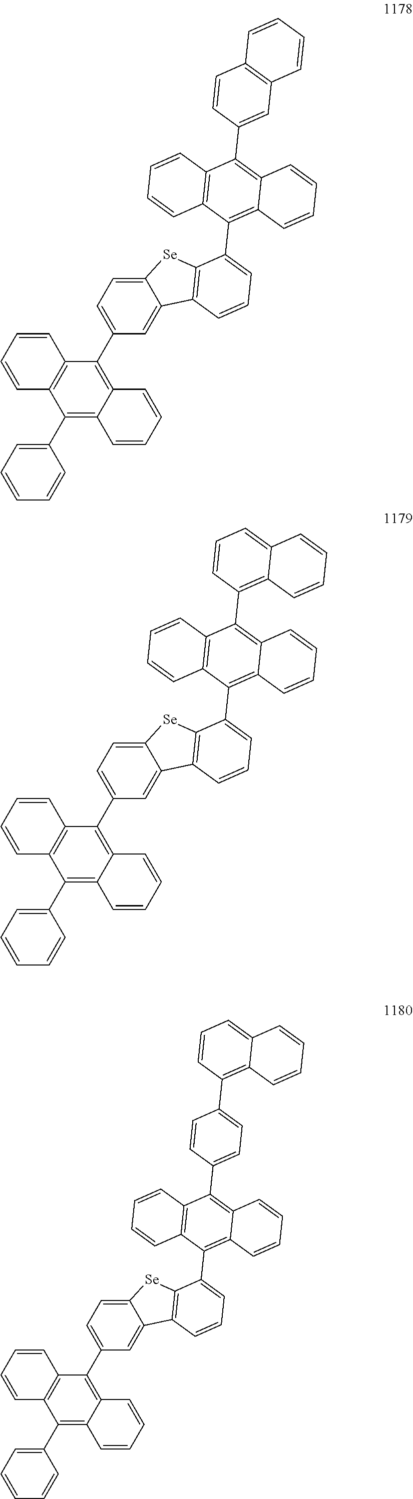 [SMILES: c1ccc(-c2c3ccccc3c(-c3ccc4[se]c5c(-c6c7ccccc7c(-c7ccc8ccccc8c7)c7ccccc67)cccc5c4c3)c3ccccc23)cc1 ; c1ccc(-c2c3ccccc3c(-c3ccc4[se]c5c(-c6c7ccccc7c(-c7cccc8ccccc78)c7ccccc67)cccc5c4c3)c3ccccc23)cc1 ; c1ccc(-c2c3ccccc3c(-c3ccc4[se]c5c(-c6c7ccccc7c(-c7ccc(-c8cccc9ccccc89)cc7)c7ccccc67)cccc5c4c3)c3ccccc23)cc1]

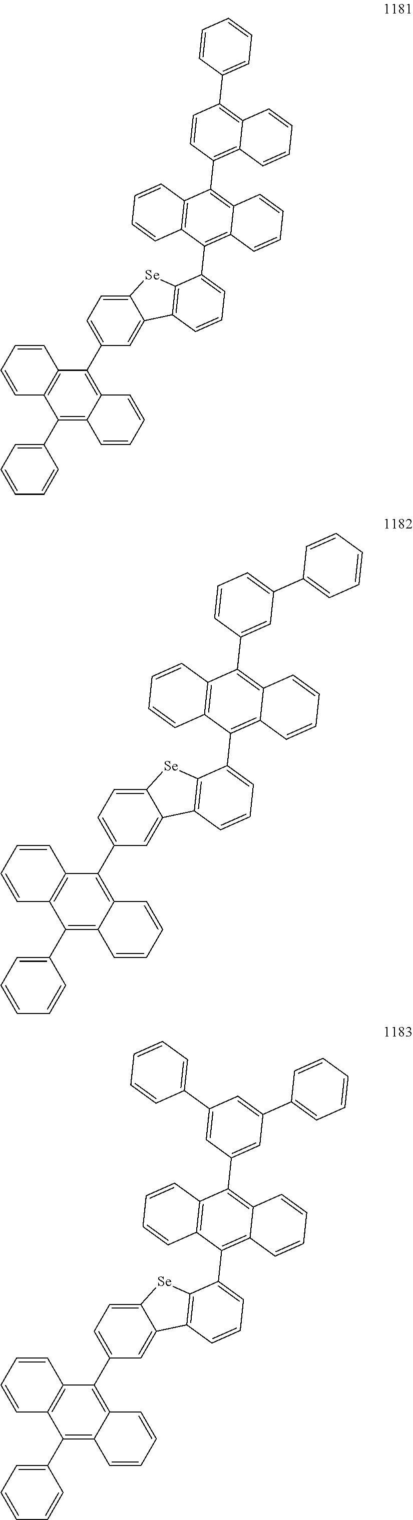 [SMILES: c1ccc(-c2ccc(-c3c4ccccc4c(-c4cccc5c4[se]c4ccc(-c6c7ccccc7c(-c7ccccc7)c7ccccc67)cc45)c4ccccc34)c3ccccc23)cc1 ; c1ccc(-c2cccc(-c3c4ccccc4c(-c4cccc5c4[se]c4ccc(-c6c7ccccc7c(-c7ccccc7)c7ccccc67)cc45)c4ccccc34)c2)cc1 ; c1ccc(-c2cc(-c3ccccc3)cc(-c3c4ccccc4c(-c4cccc5c4[se]c4ccc(-c6c7ccccc7c(-c7ccccc7)c7ccccc67)cc45)c4ccccc34)c2)cc1]

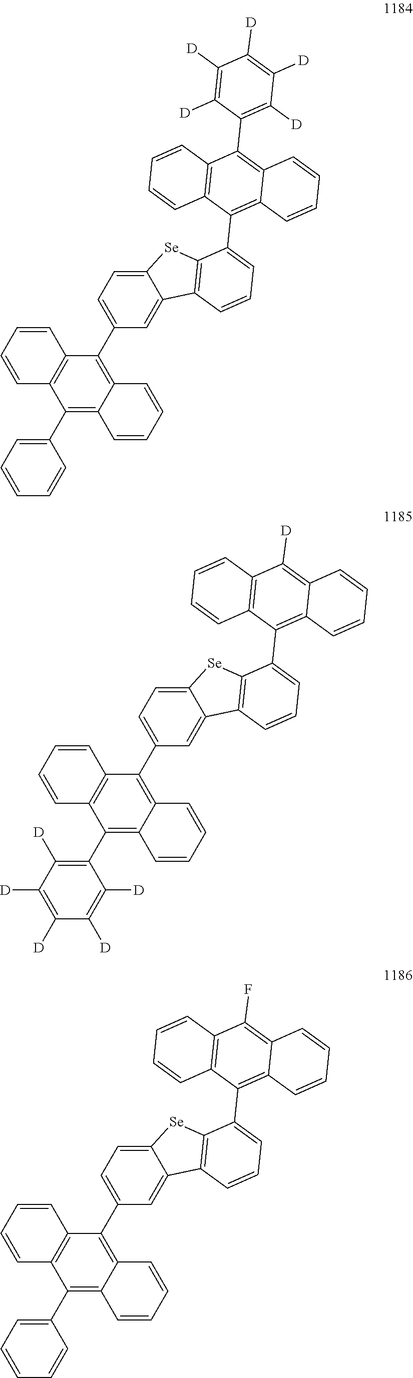 [SMILES: [2H]c1c([2H])c([2H])c(-c2c3ccccc3c(-c3cccc4c3[se]c3ccc(-c5c6ccccc6c(-c6ccccc6)c6ccccc56)cc34)c3ccccc23)c([2H])c1[2H] ; [2H]c1c([2H])c([2H])c(-c2c3ccccc3c(-c3ccc4[se]c5c(-c6c7ccccc7c([2H])c7ccccc67)cccc5c4c3)c3ccccc23)c([2H])c1[2H] ; Fc1c2ccccc2c(-c2cccc3c2[se]c2ccc(-c4c5ccccc5c(-c5ccccc5)c5ccccc45)cc23)c2ccccc12]

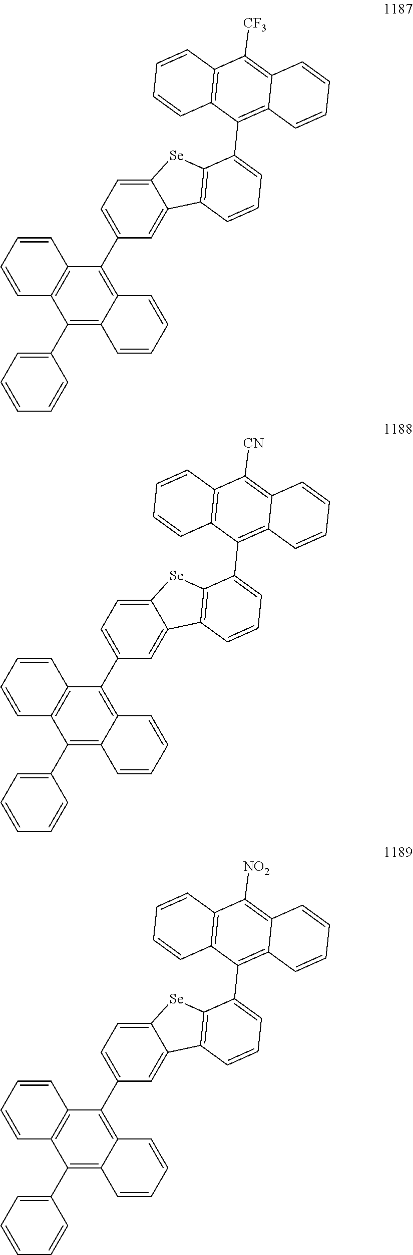 [SMILES: FC(F)(F)c1c2ccccc2c(-c2cccc3c2[se]c2ccc(-c4c5ccccc5c(-c5ccccc5)c5ccccc45)cc23)c2ccccc12 ; N#Cc1c2ccccc2c(-c2cccc3c2[se]c2ccc(-c4c5ccccc5c(-c5ccccc5)c5ccccc45)cc23)c2ccccc12 ; O=[N+]([O-])c1c2ccccc2c(-c2cccc3c2[se]c2ccc(-c4c5ccccc5c(-c5ccccc5)c5ccccc45)cc23)c2ccccc12]

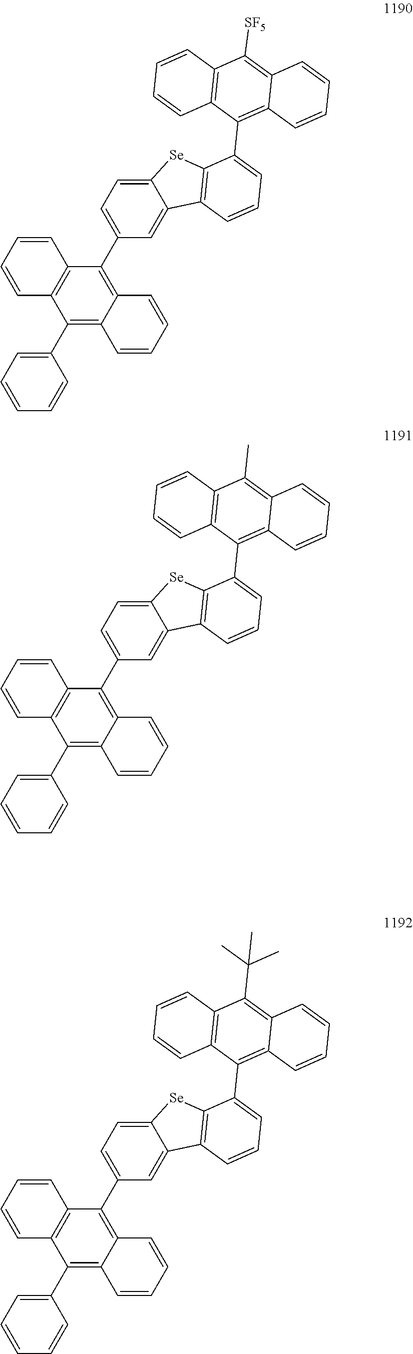

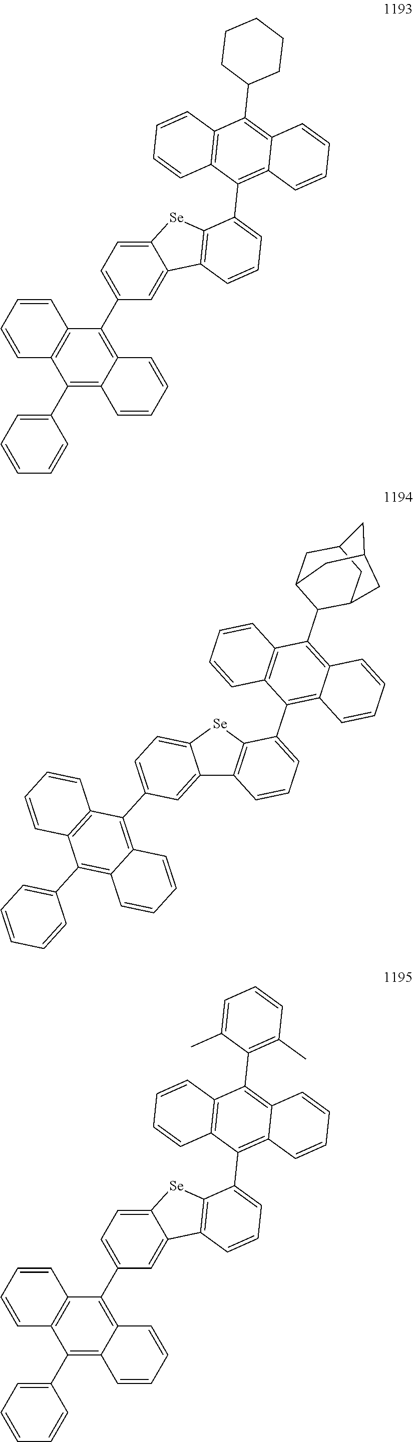 [SMILES: c1ccc(-c2c3ccccc3c(-c3ccc4[se]c5c(-c6c7ccccc7c(C7CCCCC7)c7ccccc67)cccc5c4c3)c3ccccc23)cc1 ; c1ccc(-c2c3ccccc3c(-c3ccc4[se]c5c(-c6c7ccccc7c(C7C8CC9CC(C8)CC7C9)c7ccccc67)cccc5c4c3)c3ccccc23)cc1 ; Cc1cccc(C)c1-c1c2ccccc2c(-c2cccc3c2[se]c2ccc(-c4c5ccccc5c(-c5ccccc5)c5ccccc45)cc23)c2ccccc12]

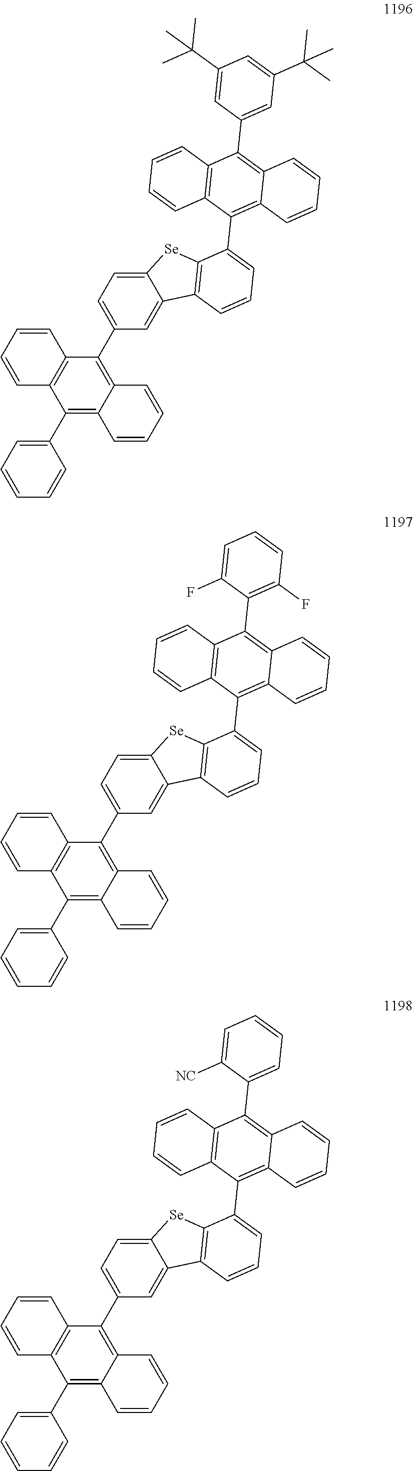 [SMILES: CC(C)(C)c1cc(-c2c3ccccc3c(-c3cccc4c3[se]c3ccc(-c5c6ccccc6c(-c6ccccc6)c6ccccc56)cc34)c3ccccc23)cc(C(C)(C)C)c1 ; Fc1cccc(F)c1-c1c2ccccc2c(-c2cccc3c2[se]c2ccc(-c4c5ccccc5c(-c5ccccc5)c5ccccc45)cc23)c2ccccc12 ; N#Cc1ccccc1-c1c2ccccc2c(-c2cccc3c2[se]c2ccc(-c4c5ccccc5c(-c5ccccc5)c5ccccc45)cc23)c2ccccc12]

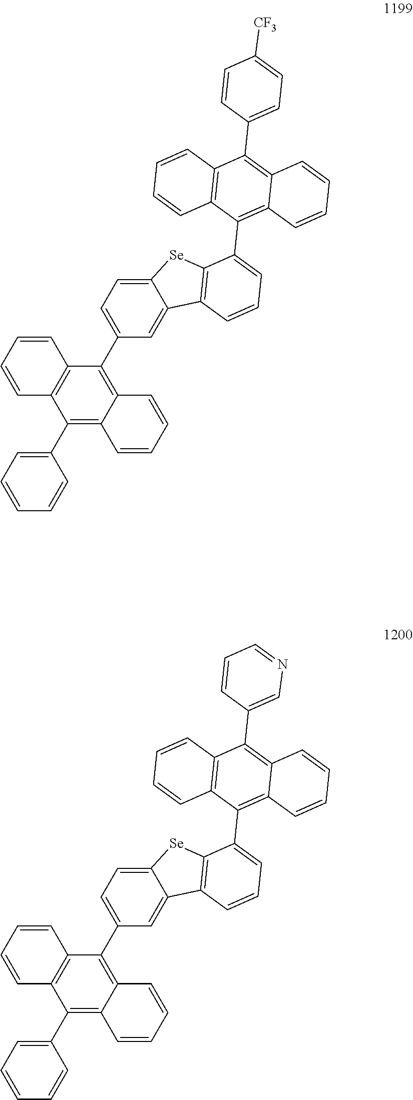 [SMILES: FC(F)(F)c1ccc(-c2c3ccccc3c(-c3cccc4c3[se]c3ccc(-c5c6ccccc6c(-c6ccccc6)c6ccccc56)cc34)c3ccccc23)cc1 ; c1ccc(-c2c3ccccc3c(-c3ccc4[se]c5c(-c6c7ccccc7c(-c7cccnc7)c7ccccc67)cccc5c4c3)c3ccccc23)cc1]

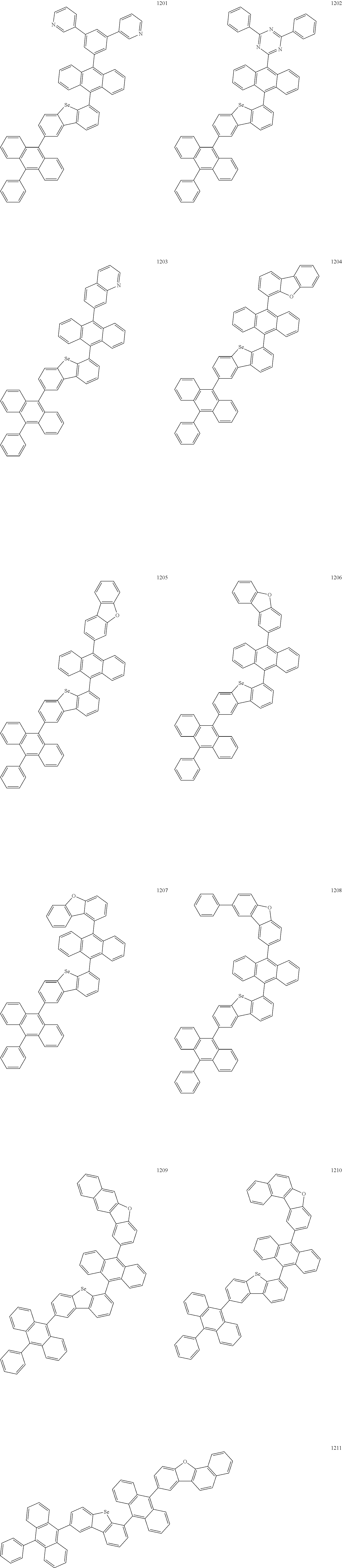

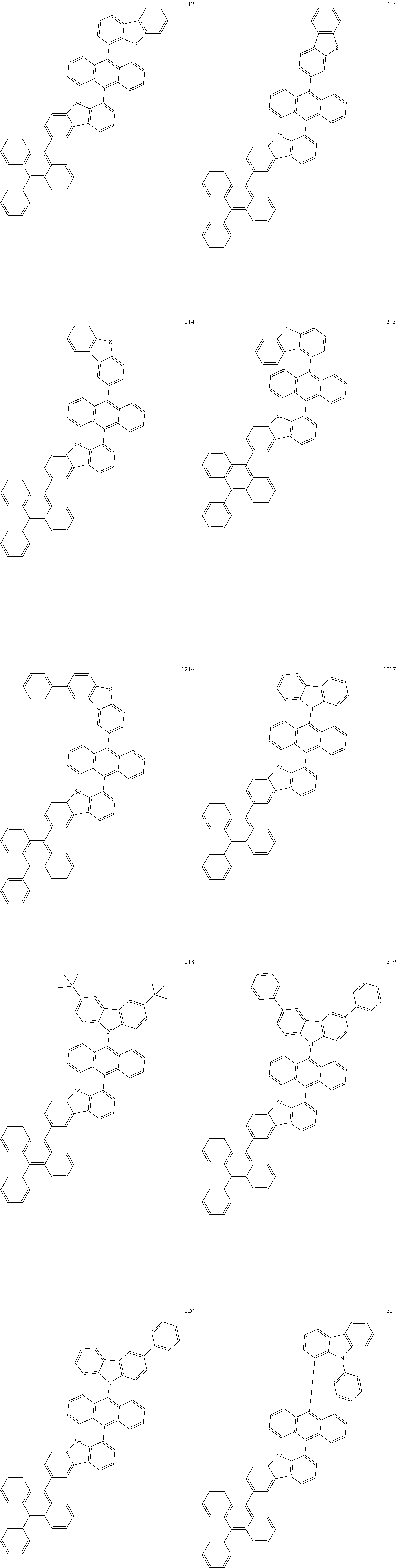

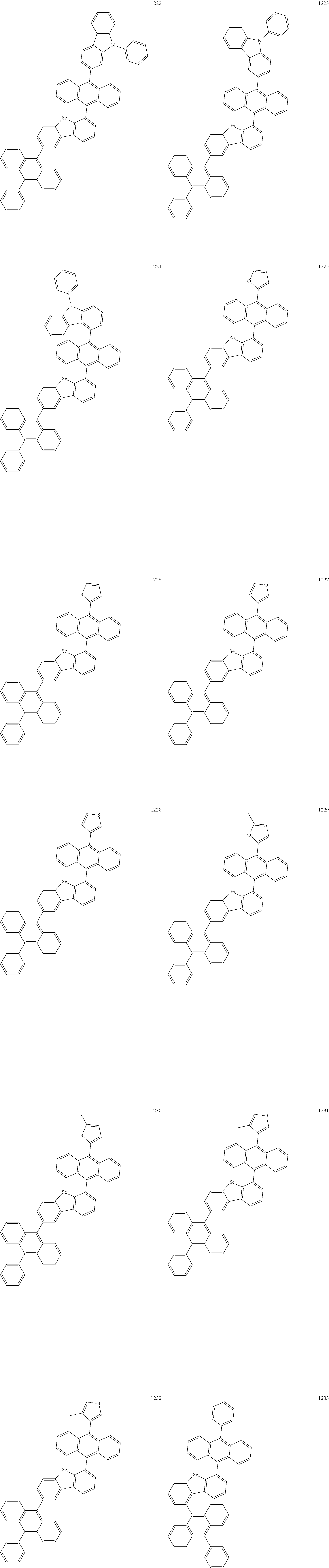

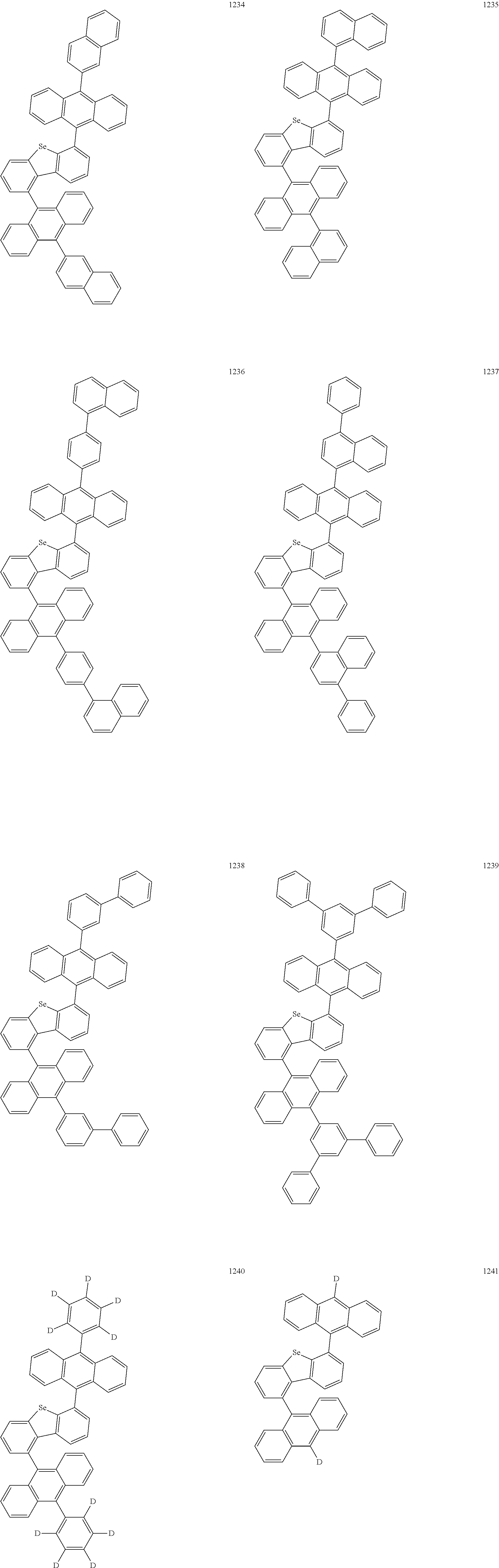

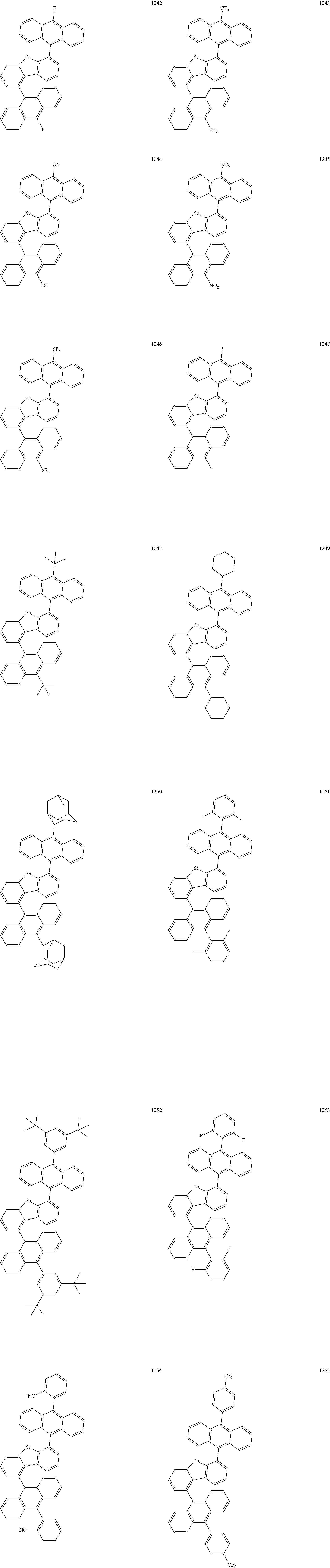

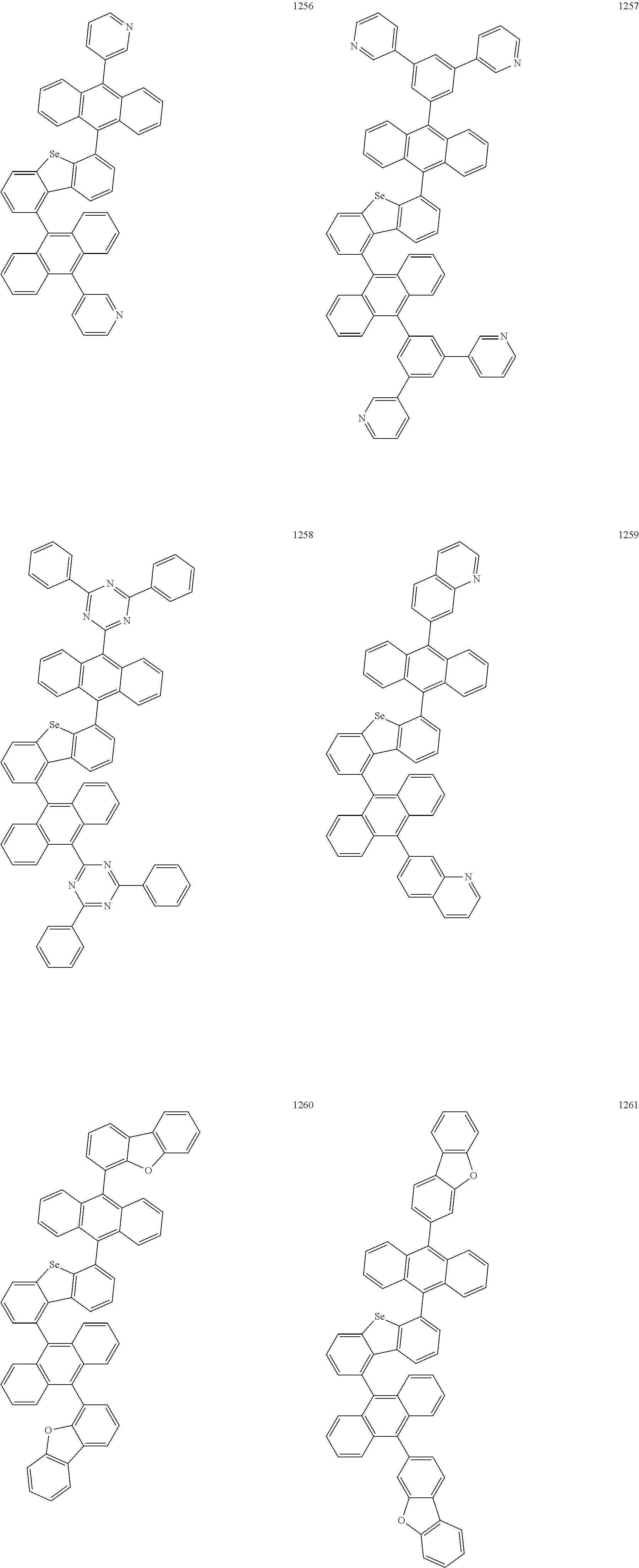

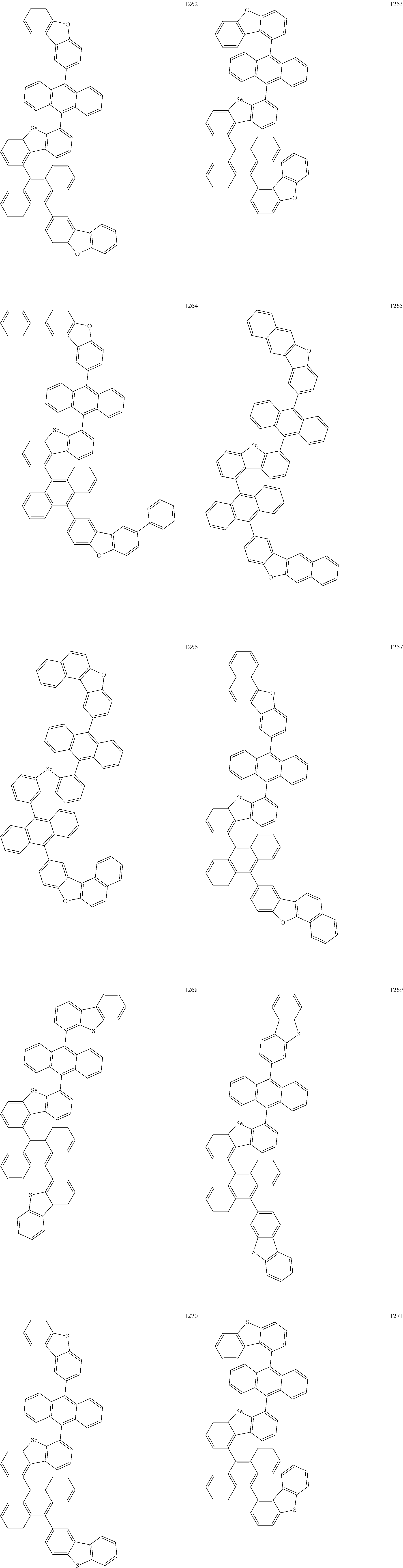

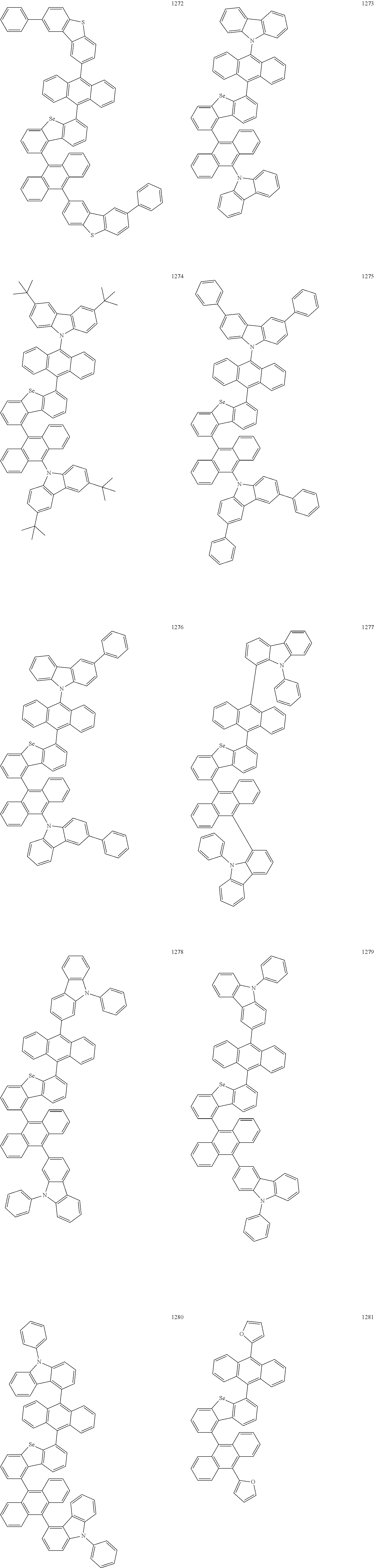

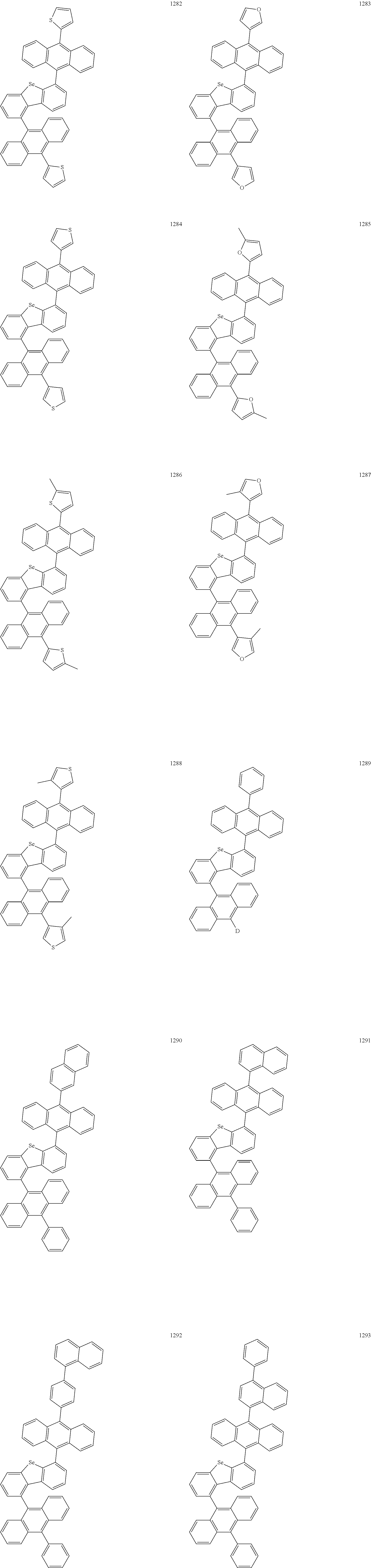

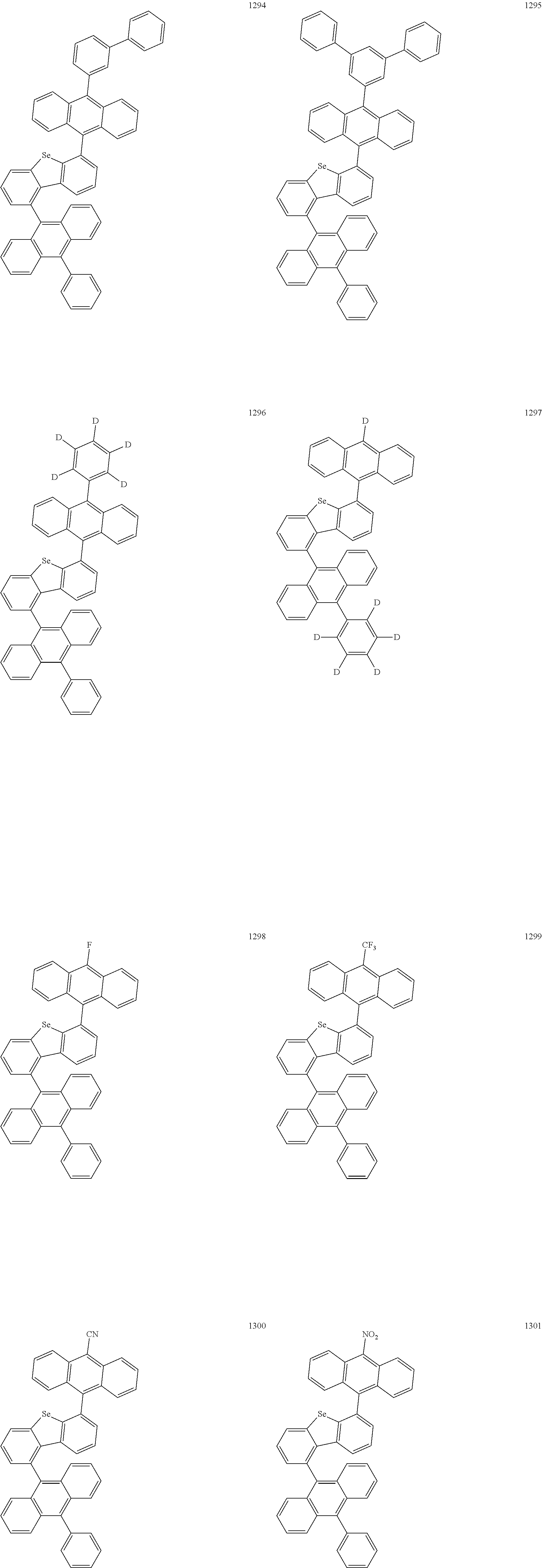

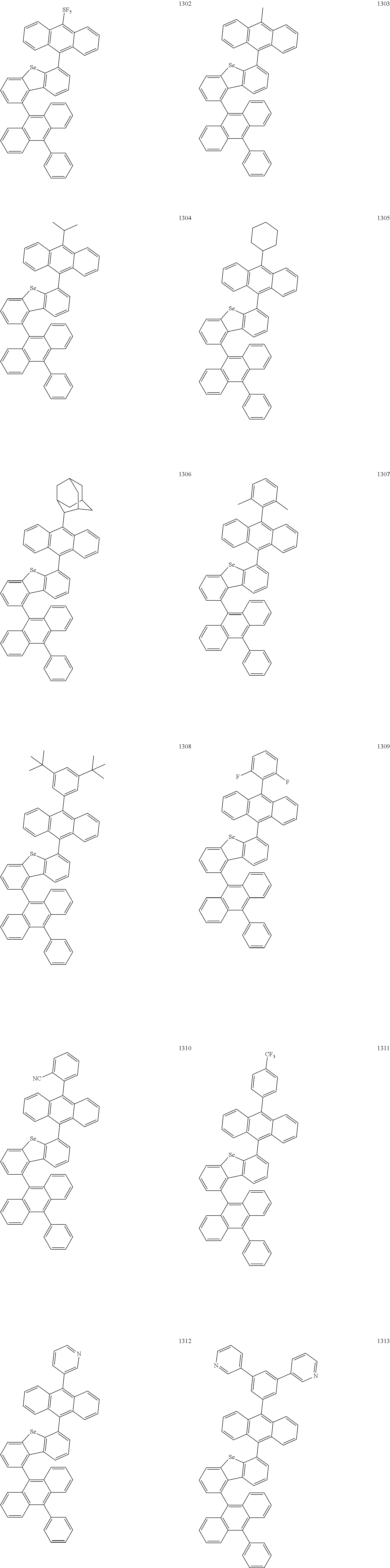

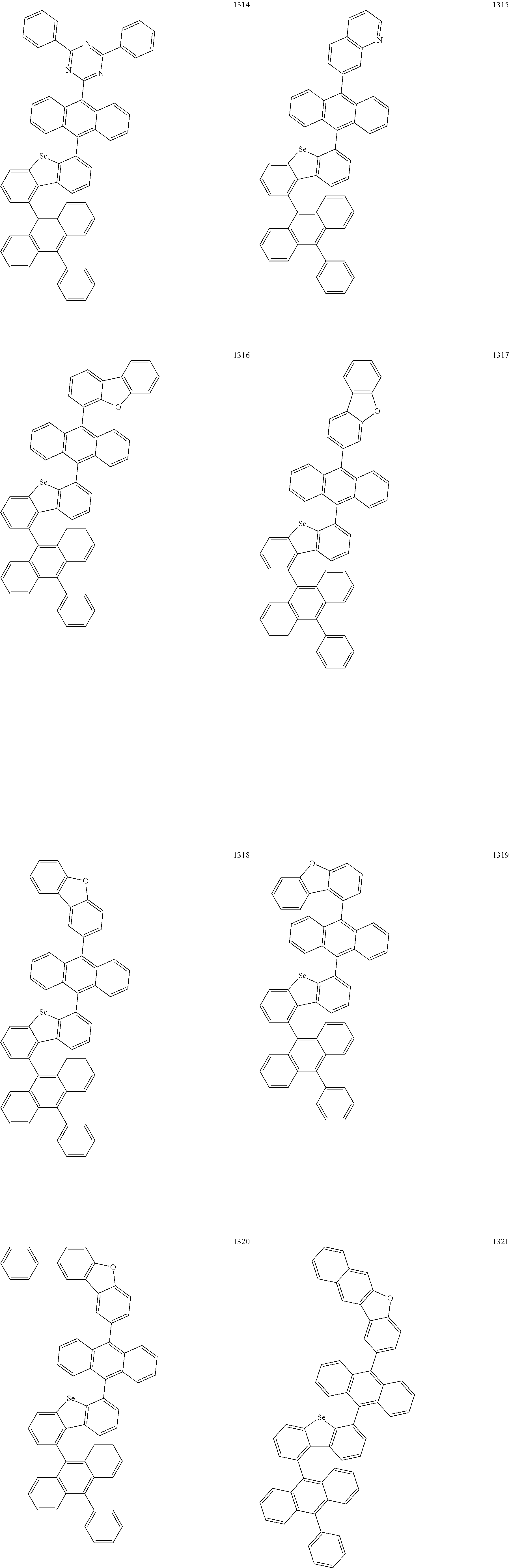

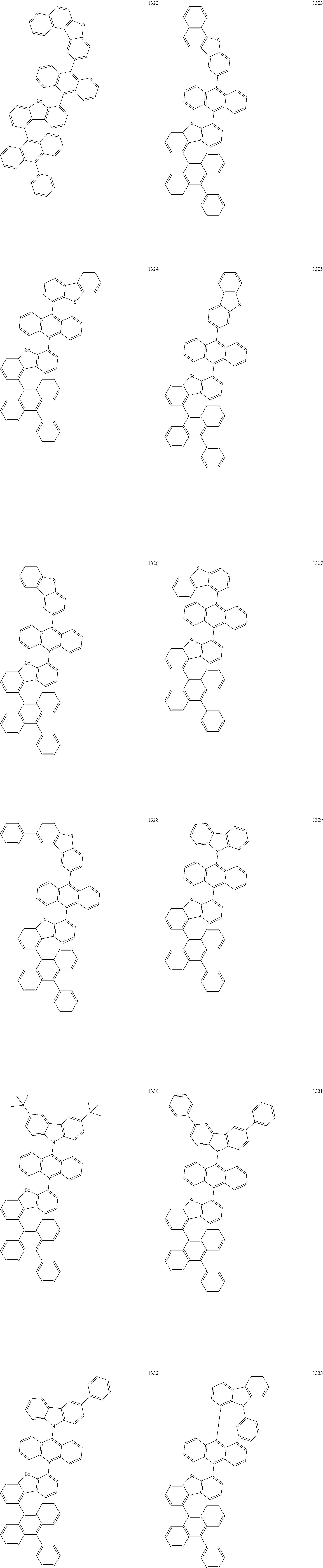

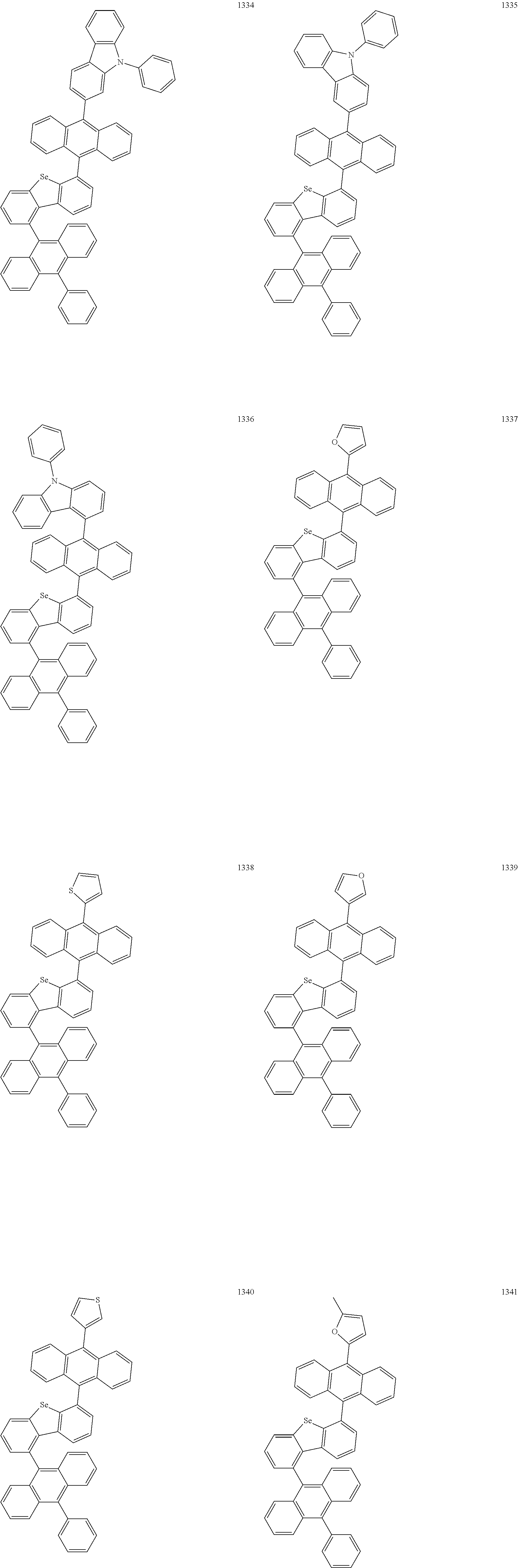

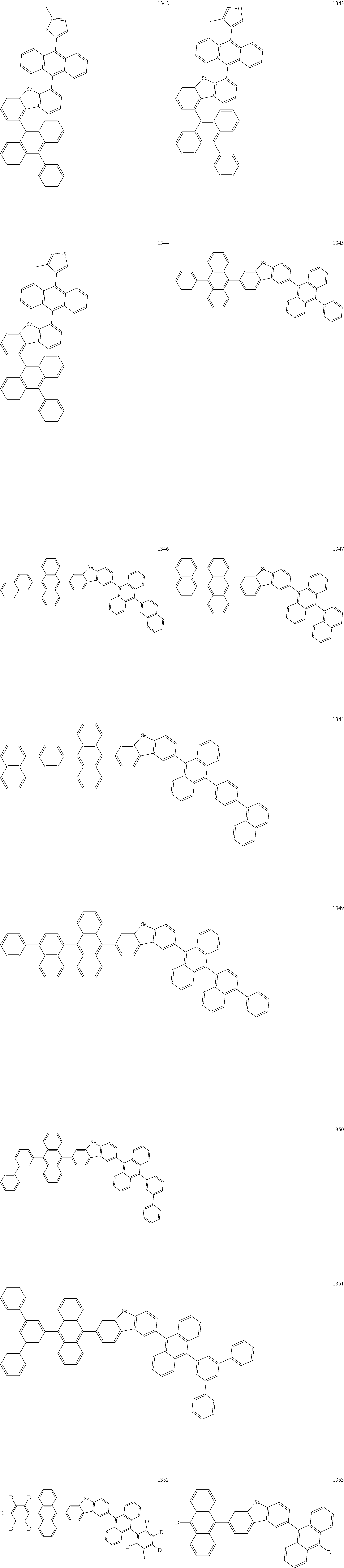

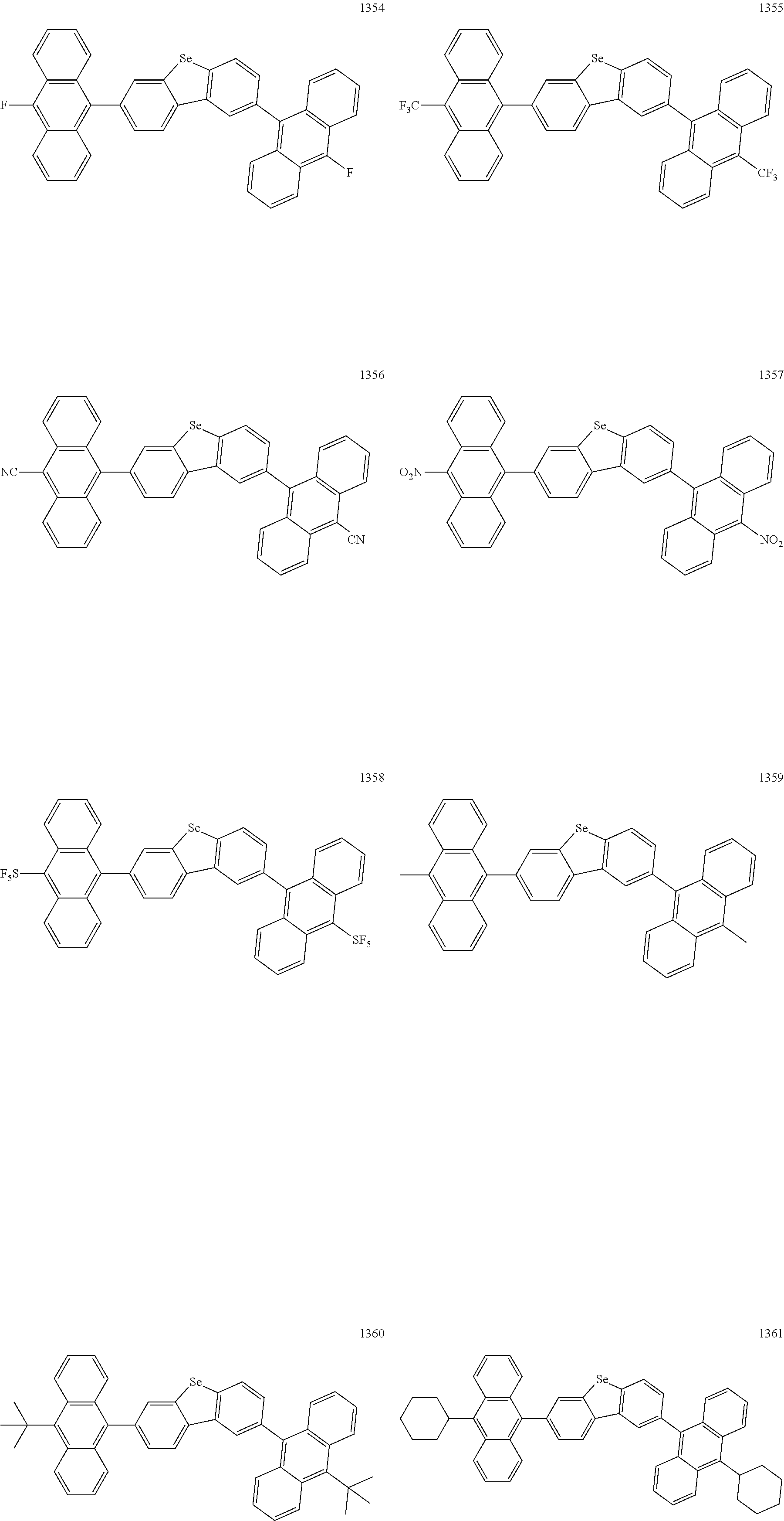

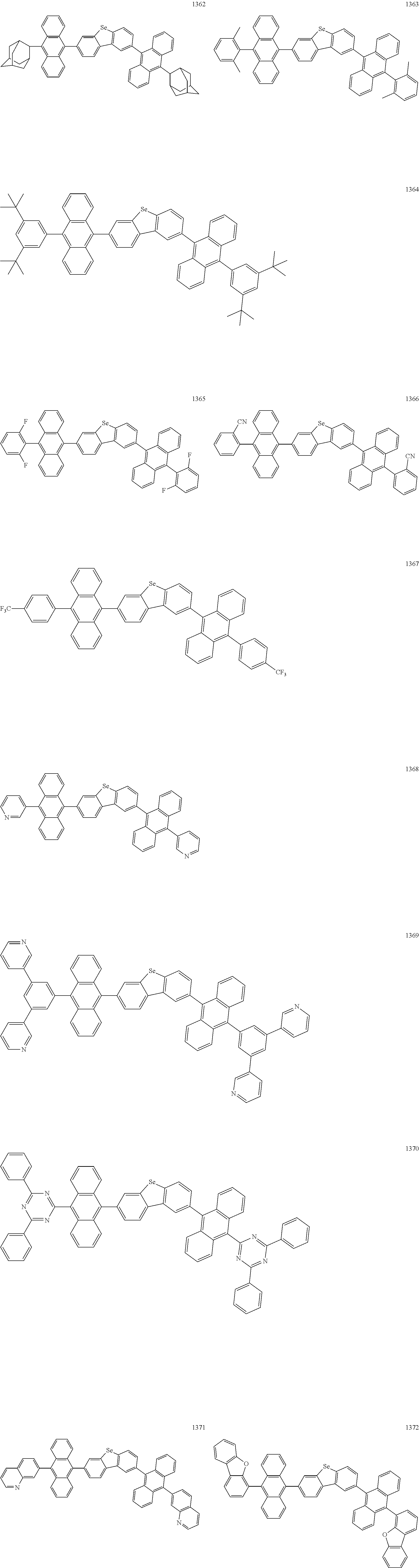

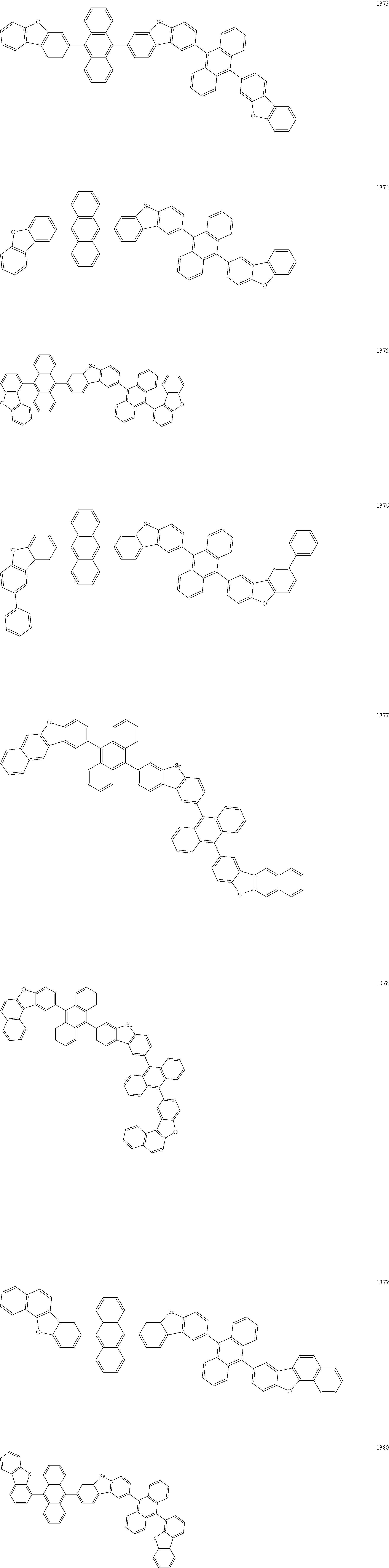

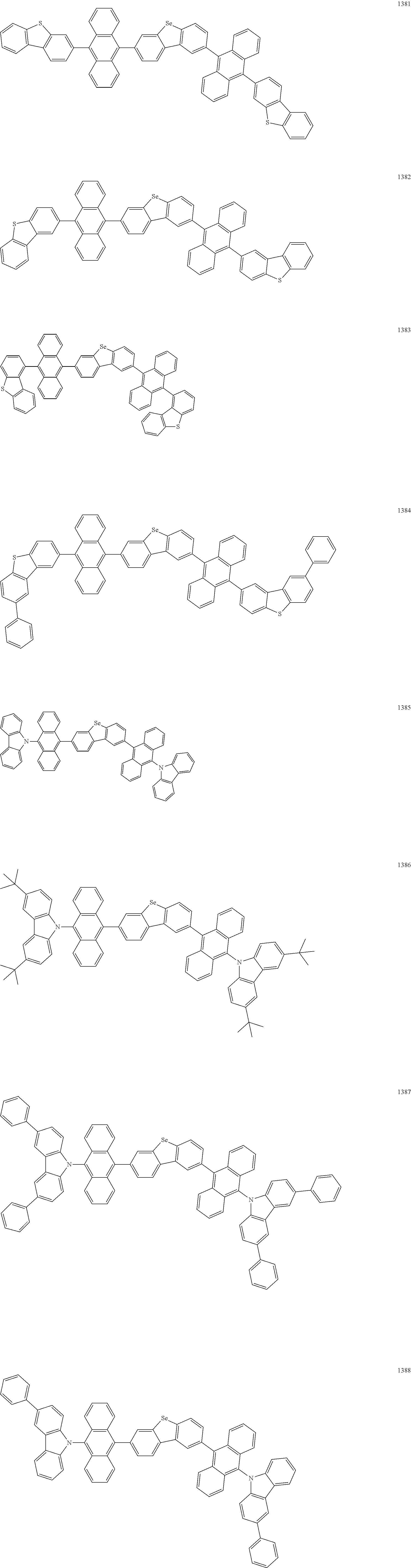

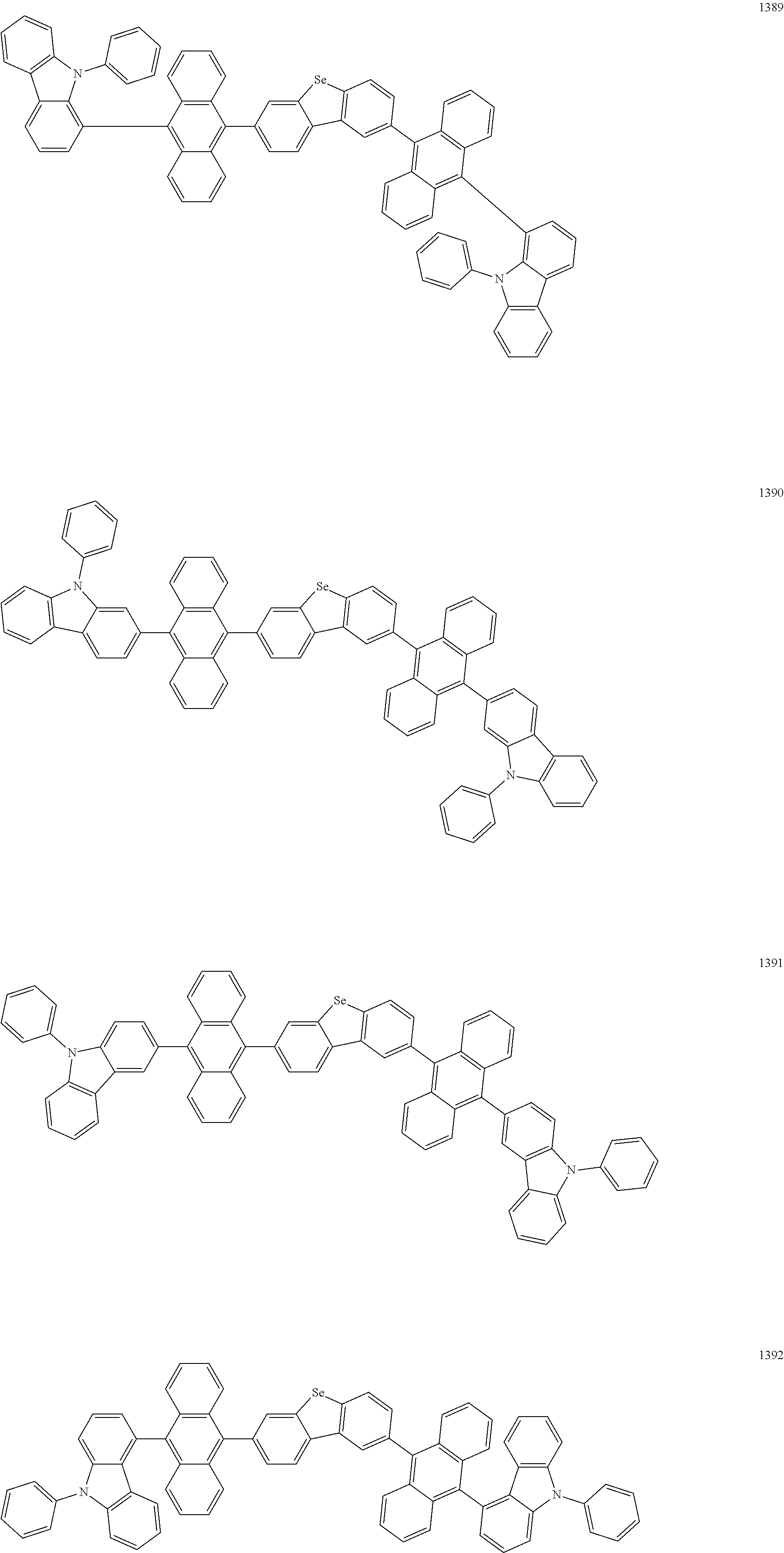

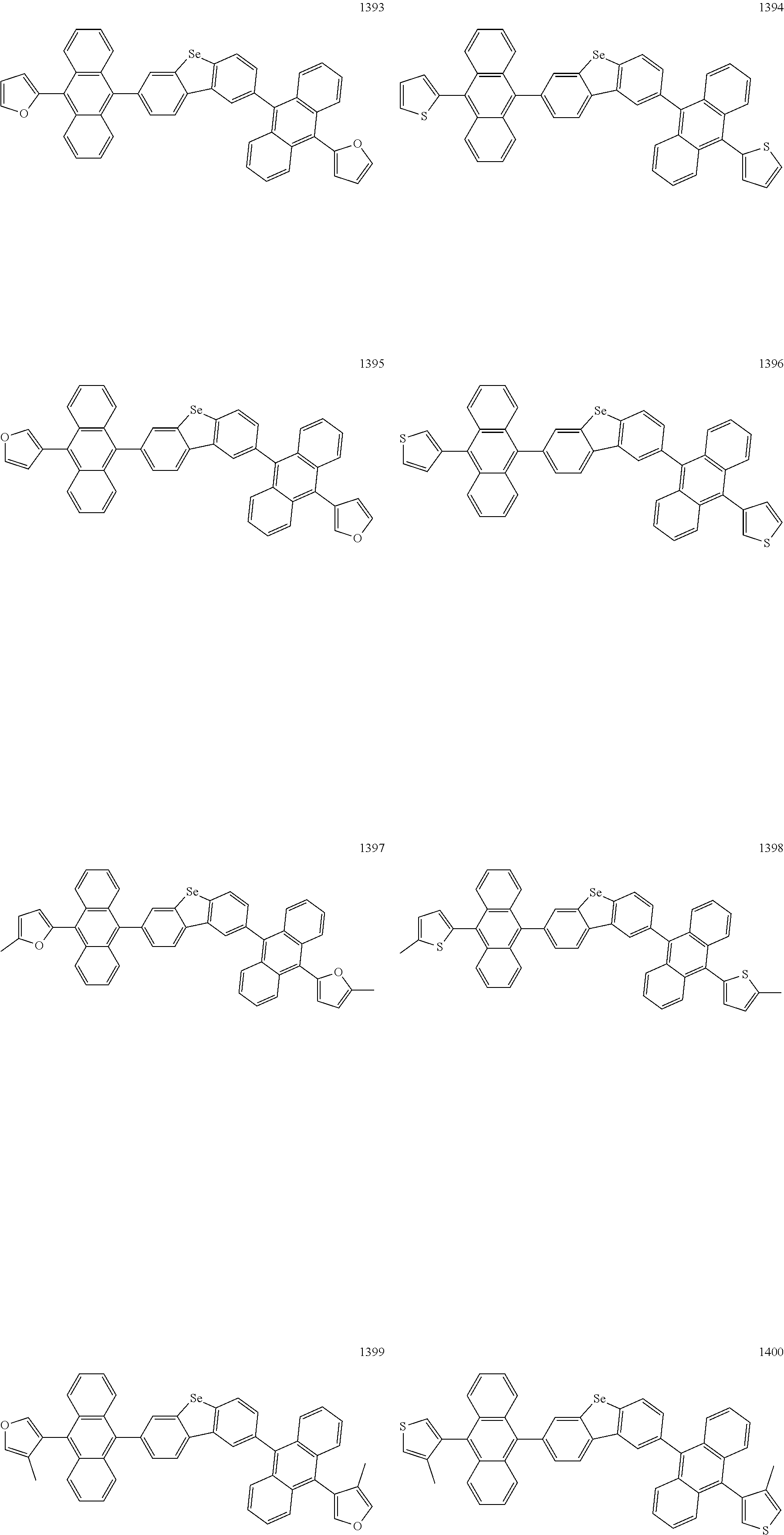

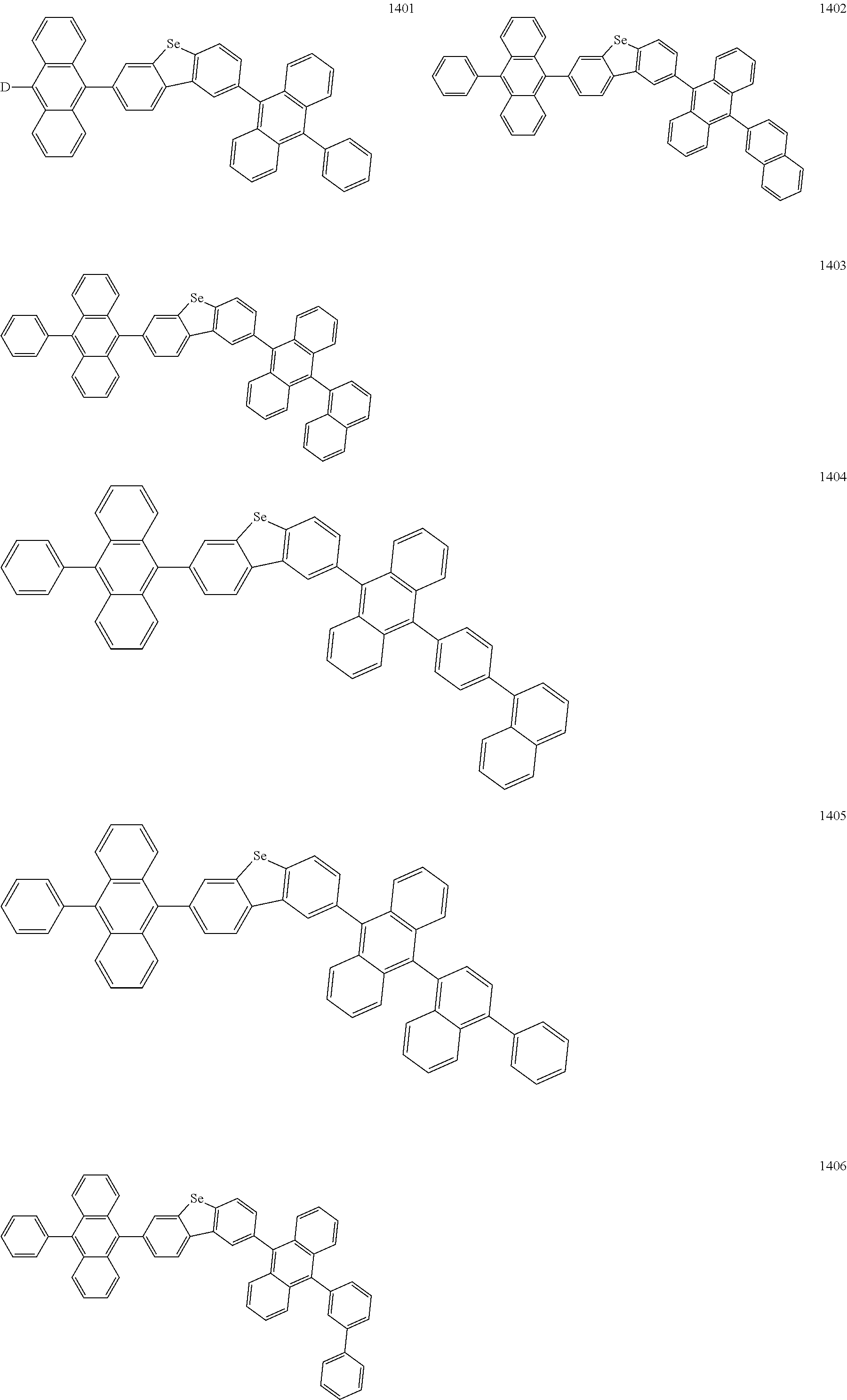

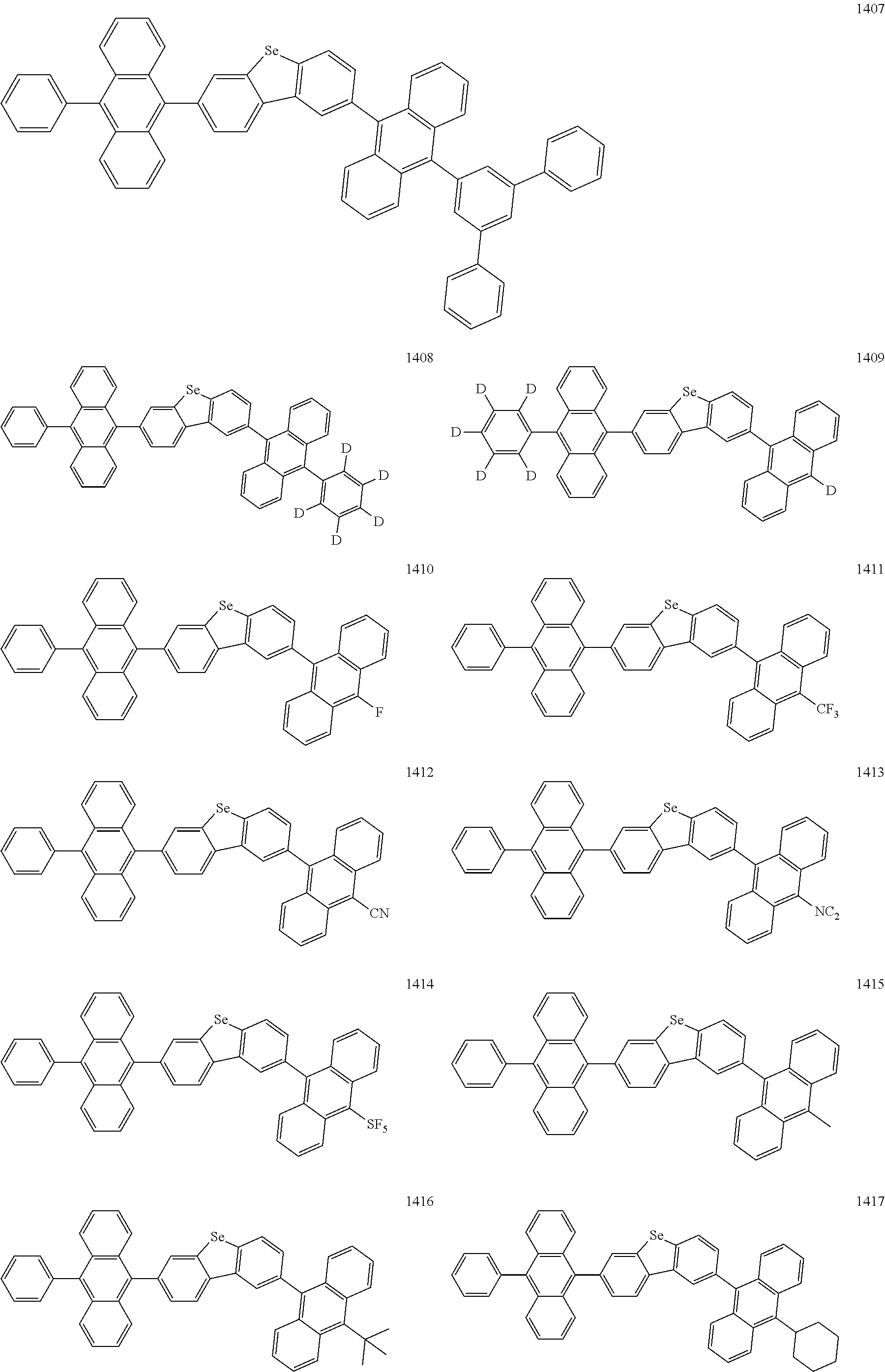

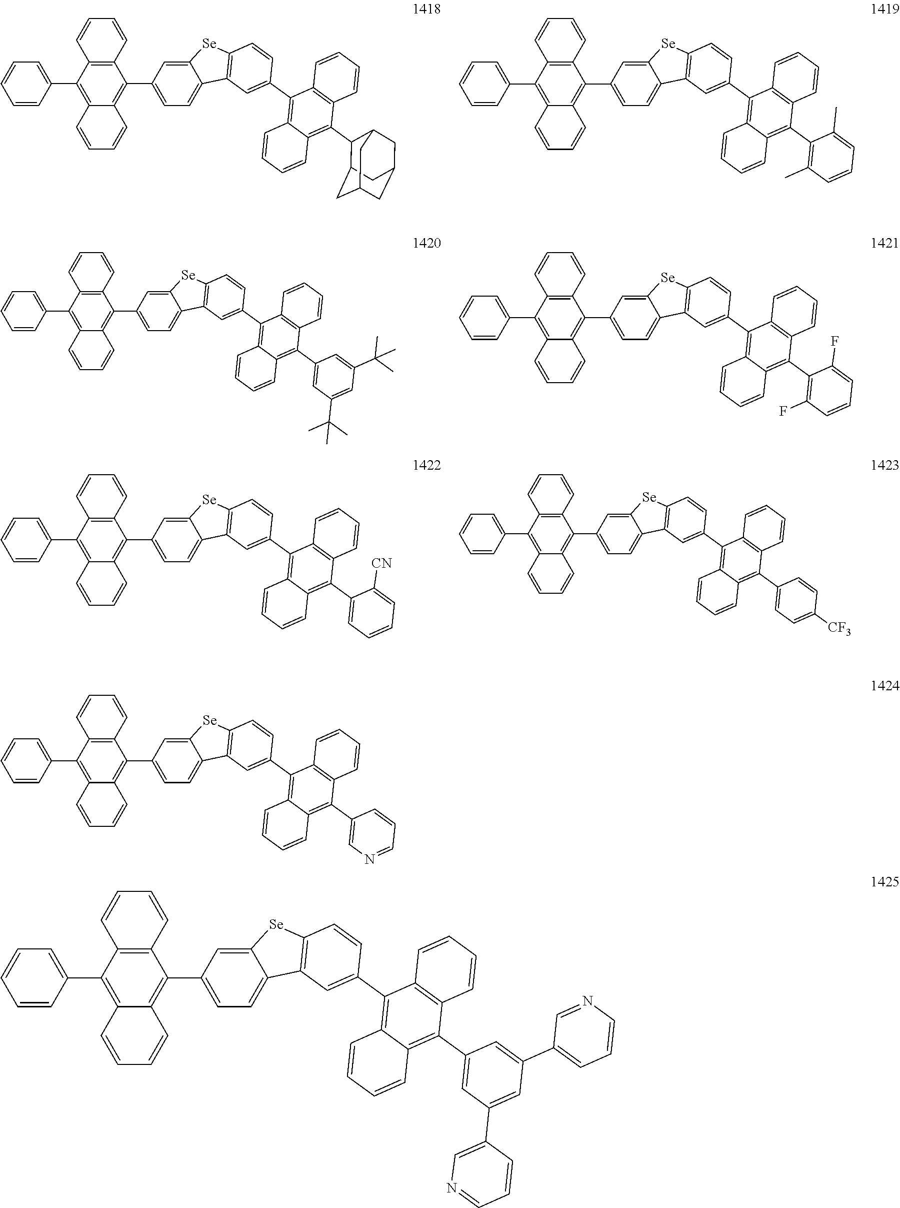

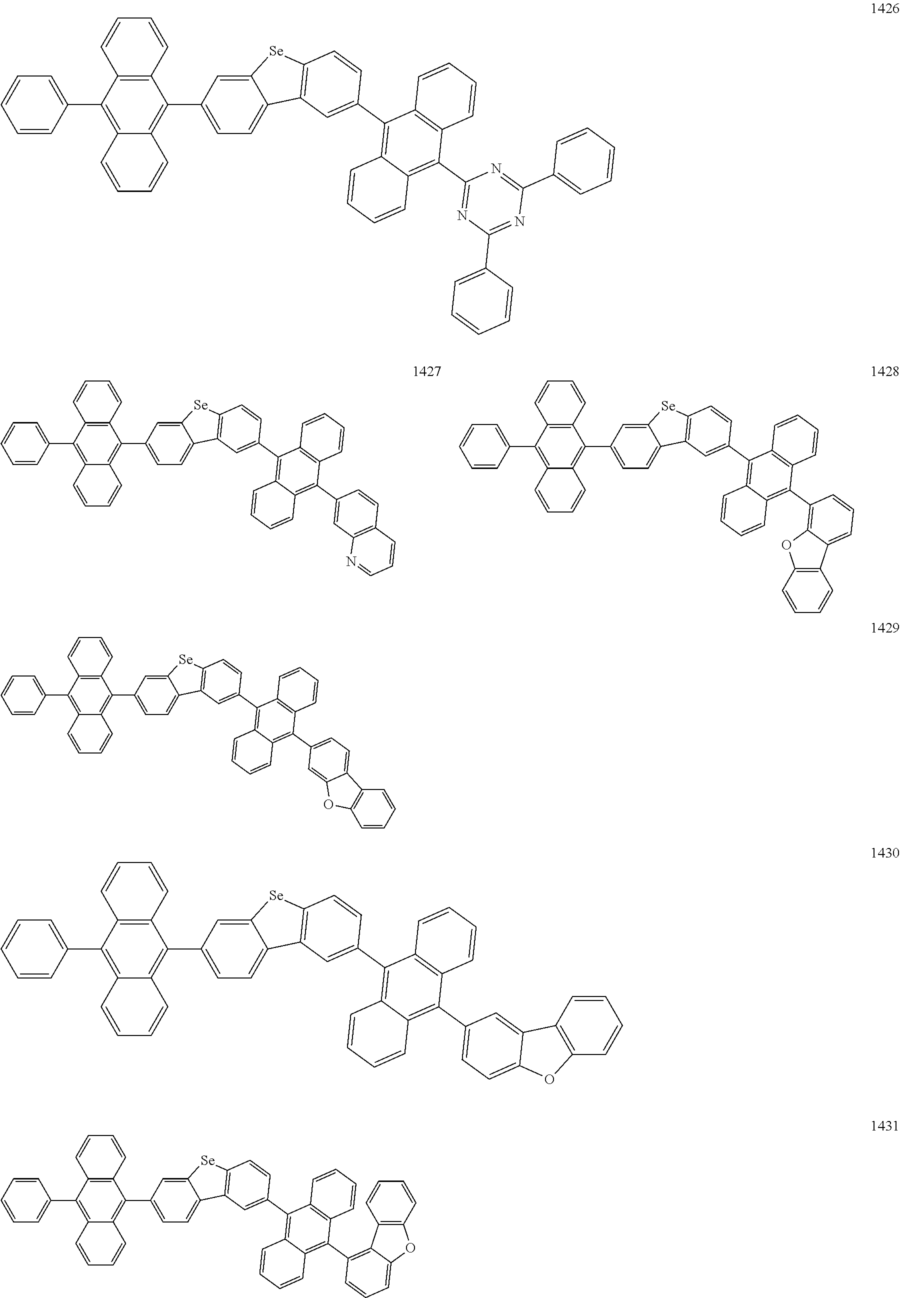

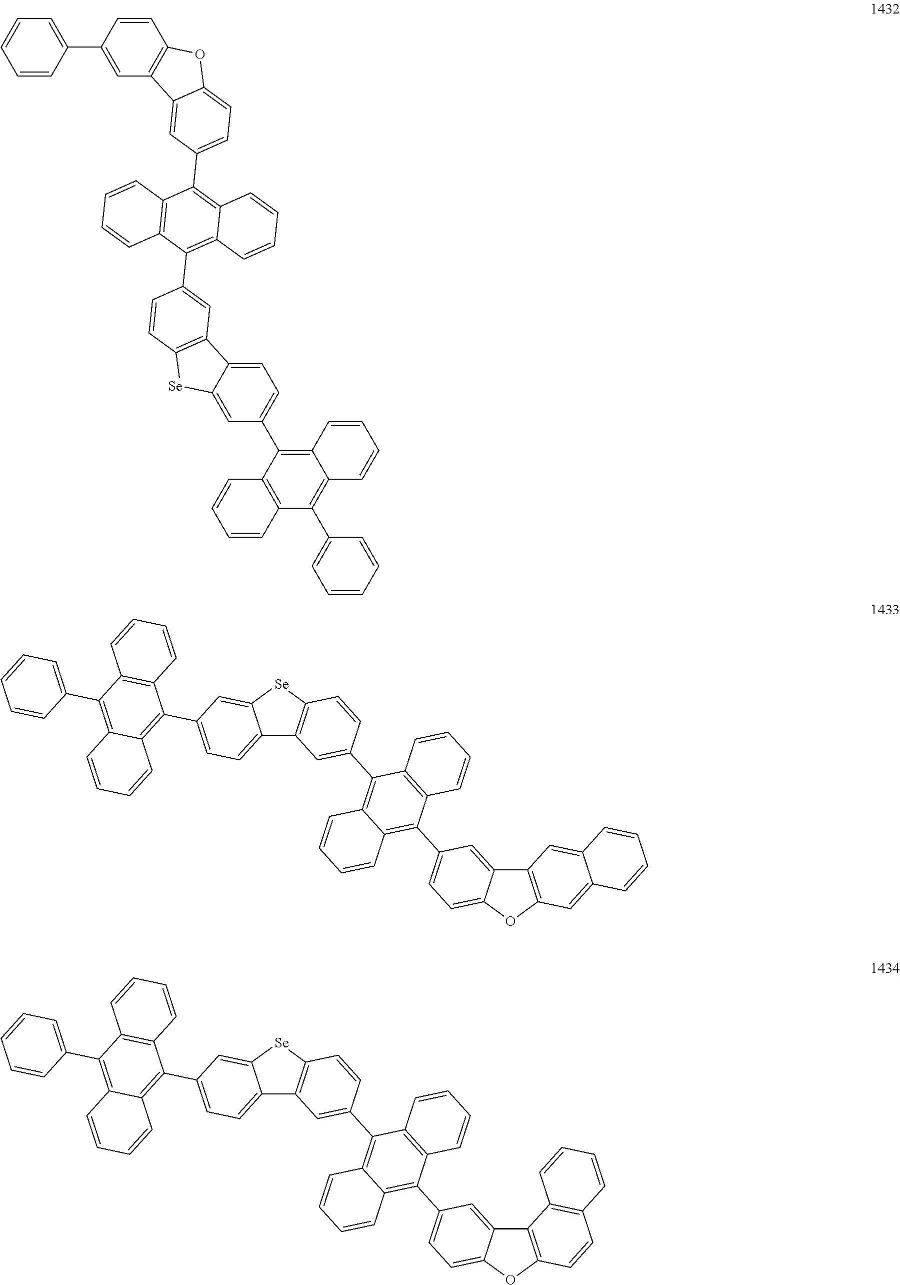 [SMILES: c1ccc(-c2ccc3oc4ccc(-c5c6ccccc6c(-c6ccc7[se]c8cc(-c9c%10ccccc%10c(-c%10ccccc%10)c%10ccccc9%10)ccc8c7c6)c6ccccc56)cc4c3c2)cc1 ; c1ccc(-c2c3ccccc3c(-c3ccc4c(c3)[se]c3ccc(-c5c6ccccc6c(-c6ccc7oc8cc9ccccc9cc8c7c6)c6ccccc56)cc34)c3ccccc23)cc1 ; c1ccc(-c2c3ccccc3c(-c3ccc4c(c3)[se]c3ccc(-c5c6ccccc6c(-c6ccc7oc8ccc9ccccc9c8c7c6)c6ccccc56)cc34)c3ccccc23)cc1]

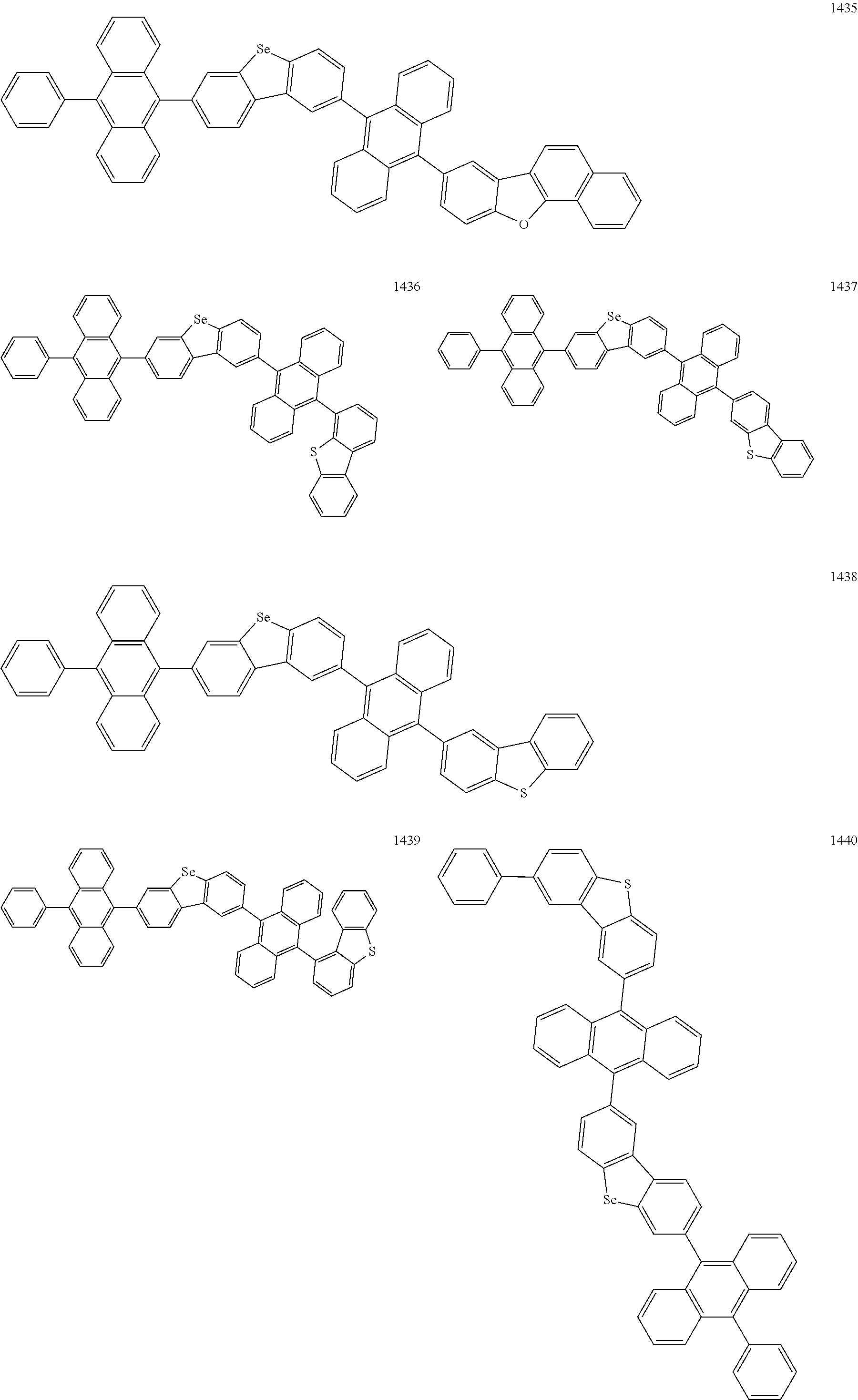

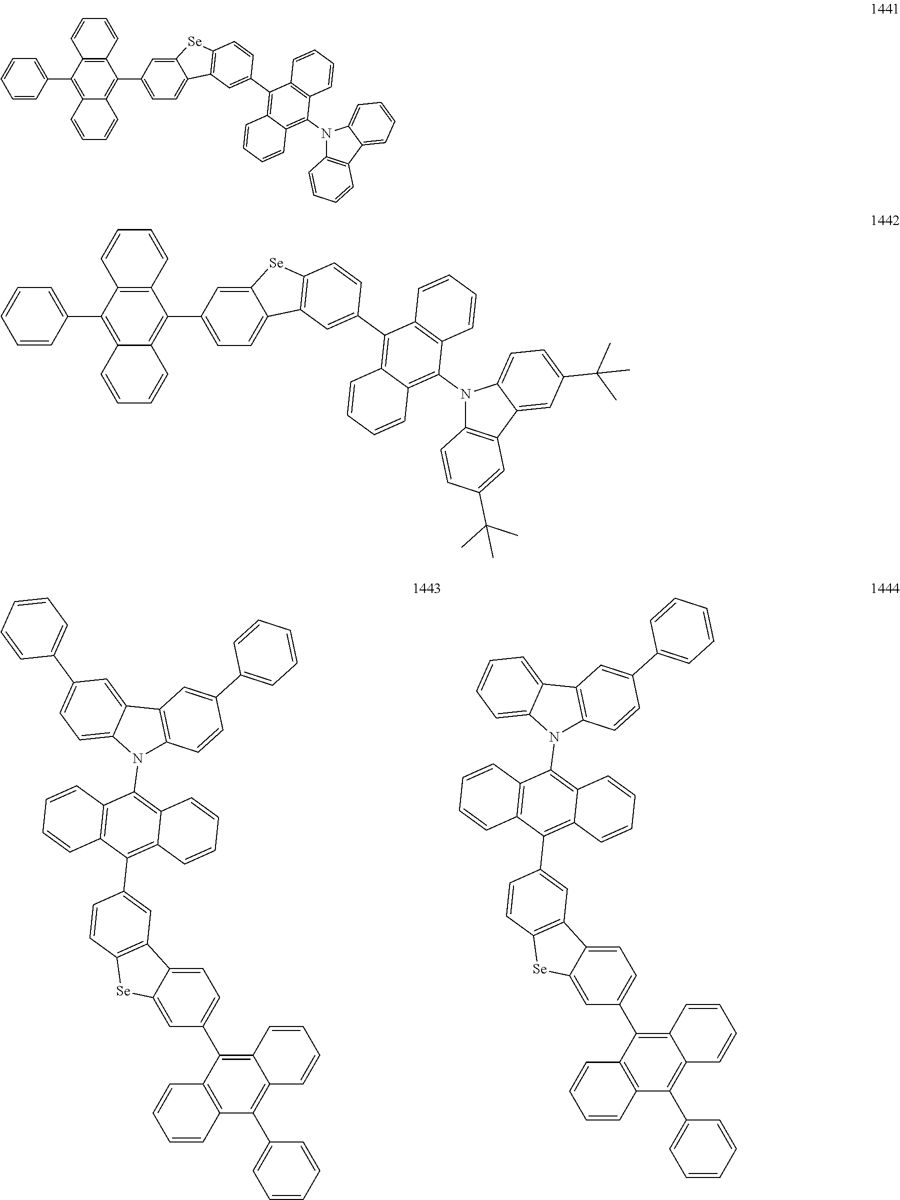 [SMILES: c1ccc(-c2c3ccccc3c(-c3ccc4c(c3)[se]c3ccc(-c5c6ccccc6c(-n6c7ccccc7c7ccccc76)c6ccccc56)cc34)c3ccccc23)cc1 ; CC(C)(C)c1ccc2c(c1)c1cc(C(C)(C)C)ccc1n2-c1c2ccccc2c(-c2ccc3[se]c4cc(-c5c6ccccc6c(-c6ccccc6)c6ccccc56)ccc4c3c2)c2ccccc12 ; c1ccc(-c2ccc3c(c2)c2cc(-c4ccccc4)ccc2n3-c2c3ccccc3c(-c3ccc4[se]c5cc(-c6c7ccccc7c(-c7ccccc7)c7ccccc67)ccc5c4c3)c3ccccc23)cc1 ; c1ccc(-c2ccc3c(c2)c2ccccc2n3-c2c3ccccc3c(-c3ccc4[se]c5cc(-c6c7ccccc7c(-c7ccccc7)c7ccccc67)ccc5c4c3)c3ccccc23)cc1]

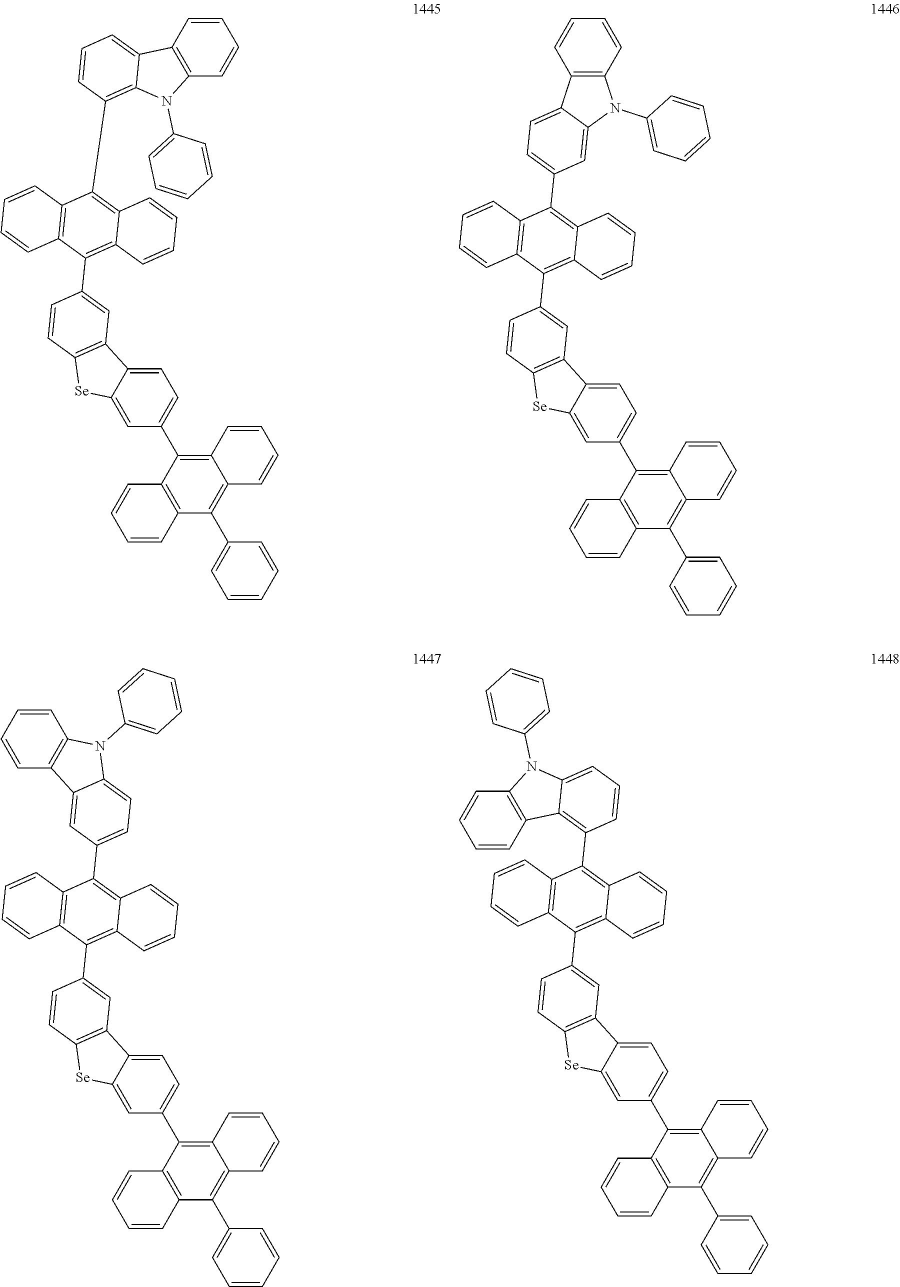 [SMILES: c1ccc(-c2c3ccccc3c(-c3ccc4c(c3)[se]c3ccc(-c5c6ccccc6c(-c6cccc7c8ccccc8n(-c8ccccc8)c67)c6ccccc56)cc34)c3ccccc23)cc1 ; c1ccc(-c2c3ccccc3c(-c3ccc4c(c3)[se]c3ccc(-c5c6ccccc6c(-c6ccc7c8ccccc8n(-c8ccccc8)c7c6)c6ccccc56)cc34)c3ccccc23)cc1 ; c1ccc(-c2c3ccccc3c(-c3ccc4c(c3)[se]c3ccc(-c5c6ccccc6c(-c6ccc7c(c6)c6ccccc6n7-c6ccccc6)c6ccccc56)cc34)c3ccccc23)cc1 ; c1ccc(-c2c3ccccc3c(-c3ccc4c(c3)[se]c3ccc(-c5c6ccccc6c(-c6cccc7c6c6ccccc6n7-c6ccccc6)c6ccccc56)cc34)c3ccccc23)cc1]

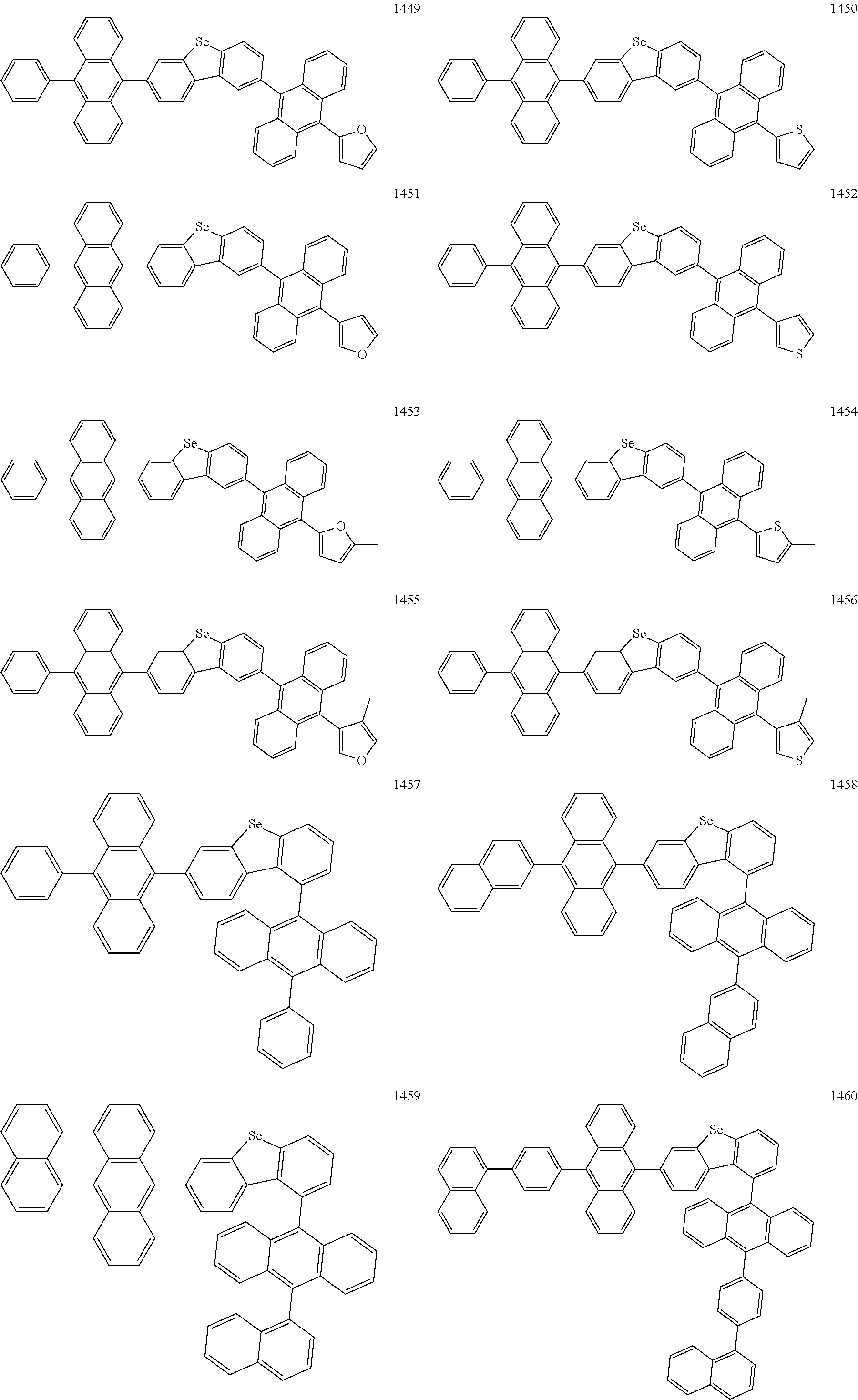

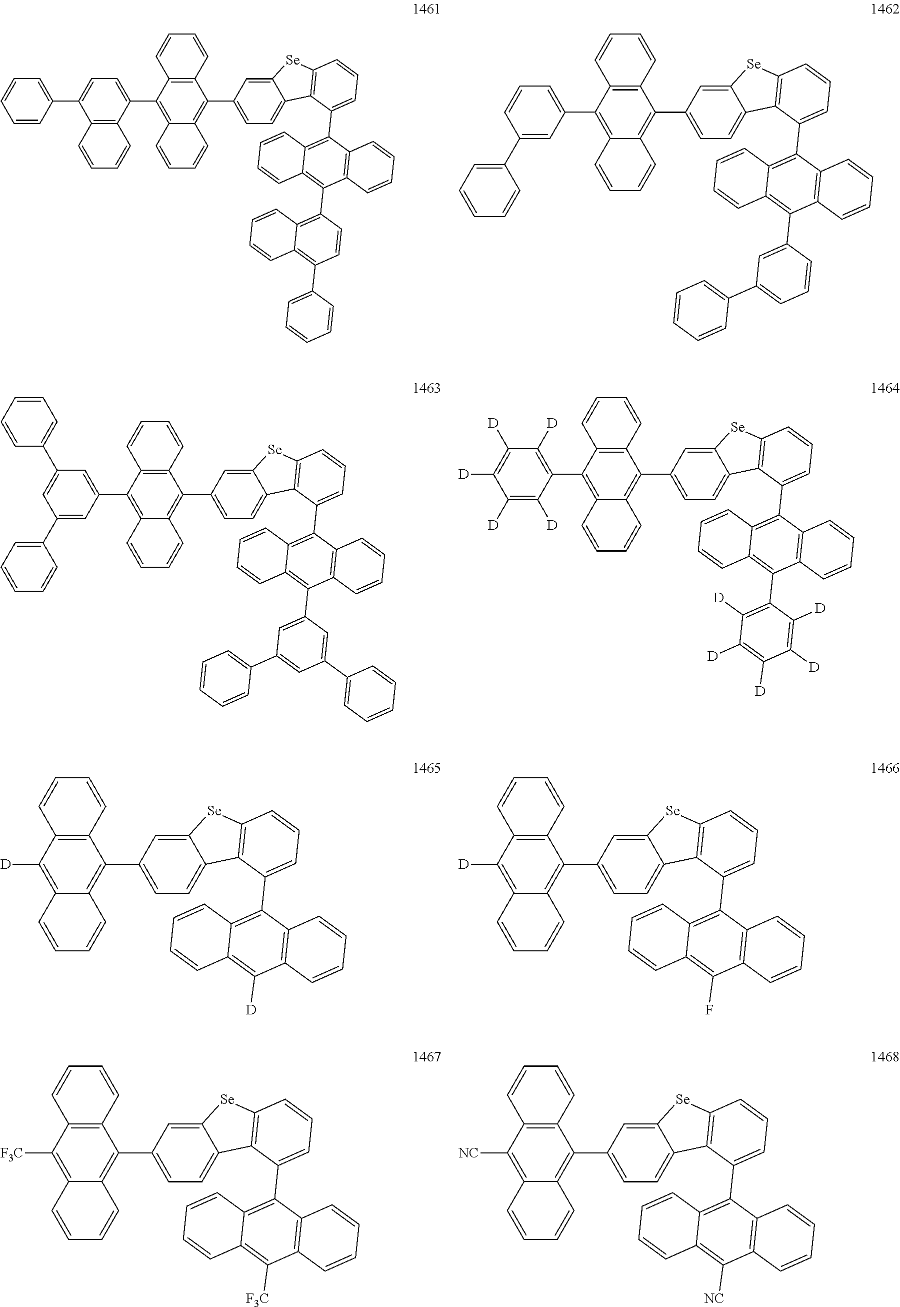

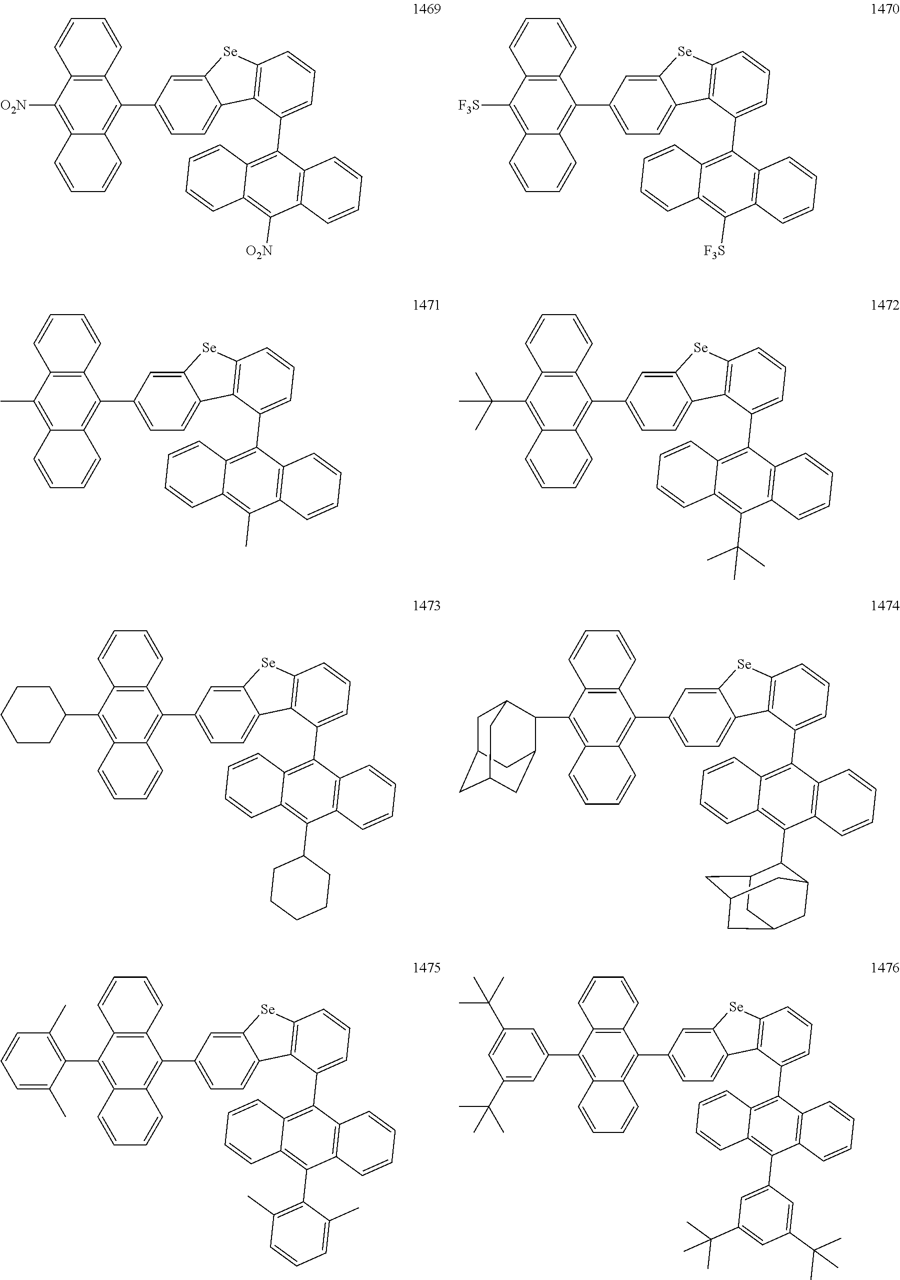

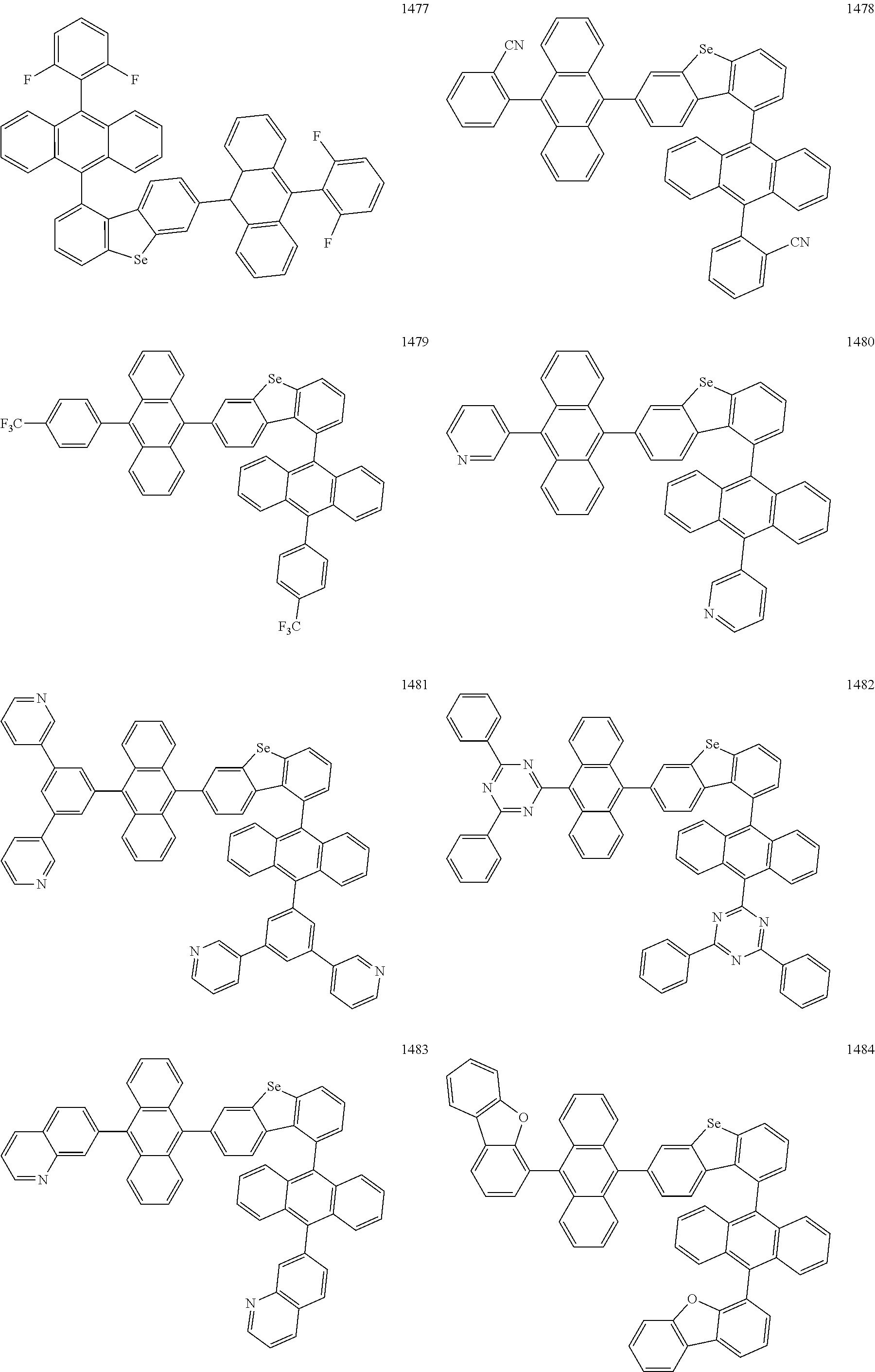

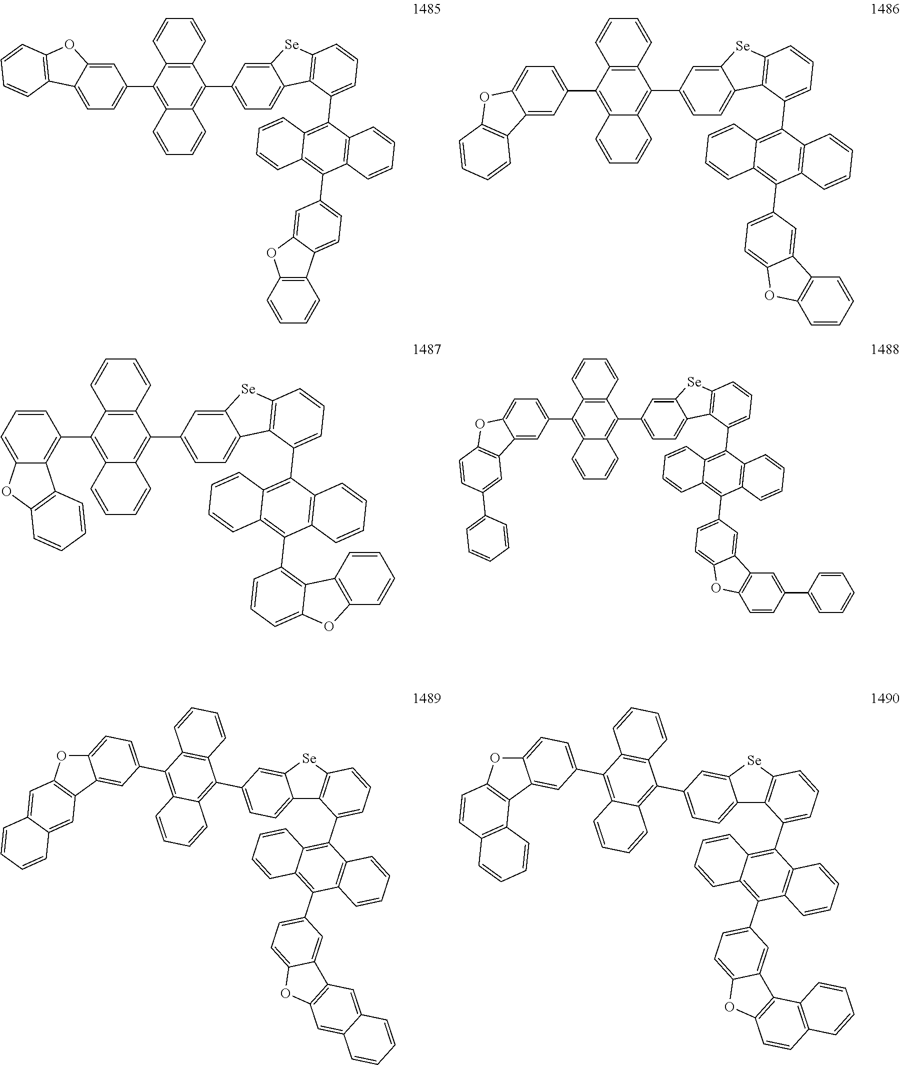

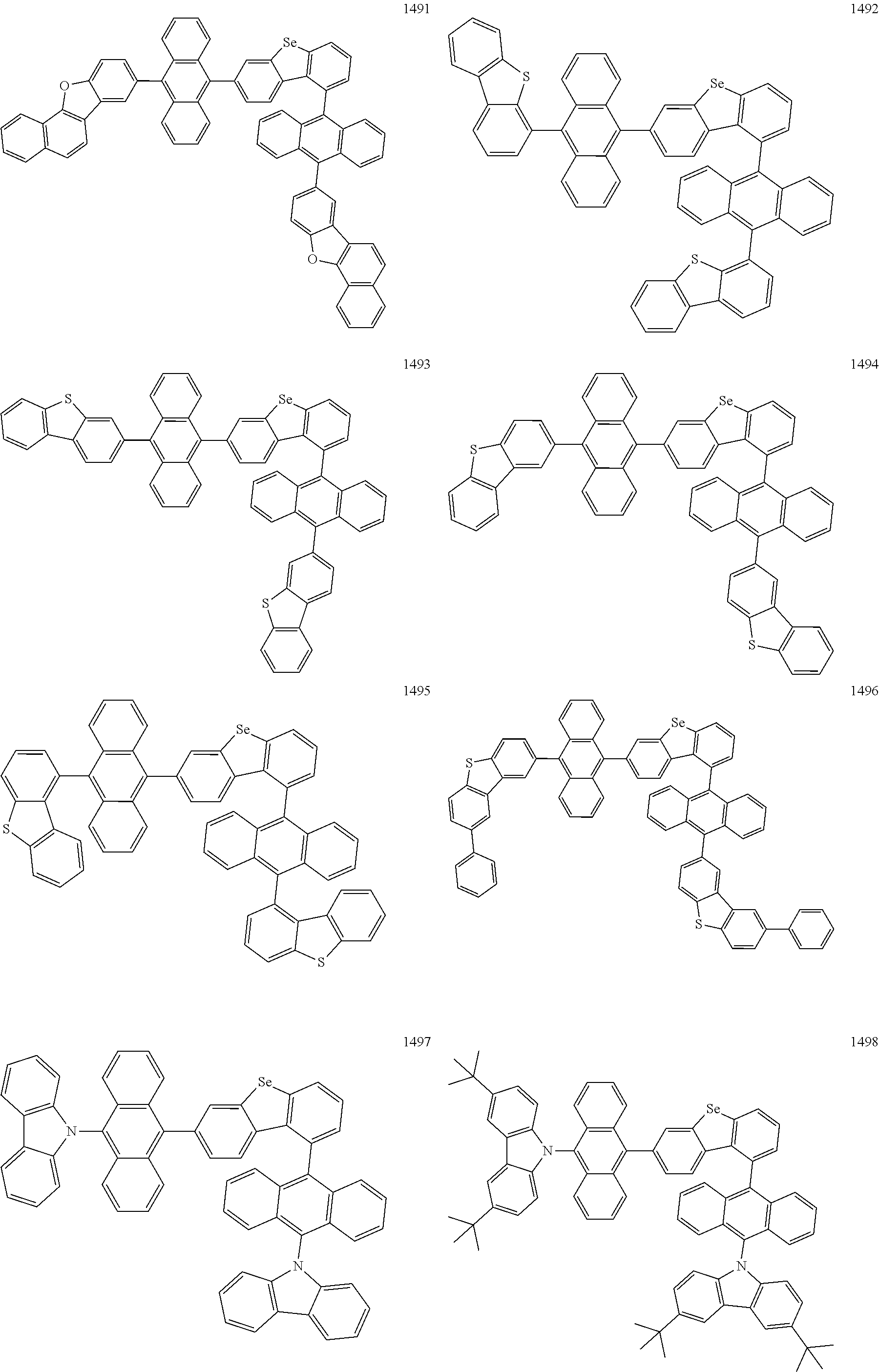

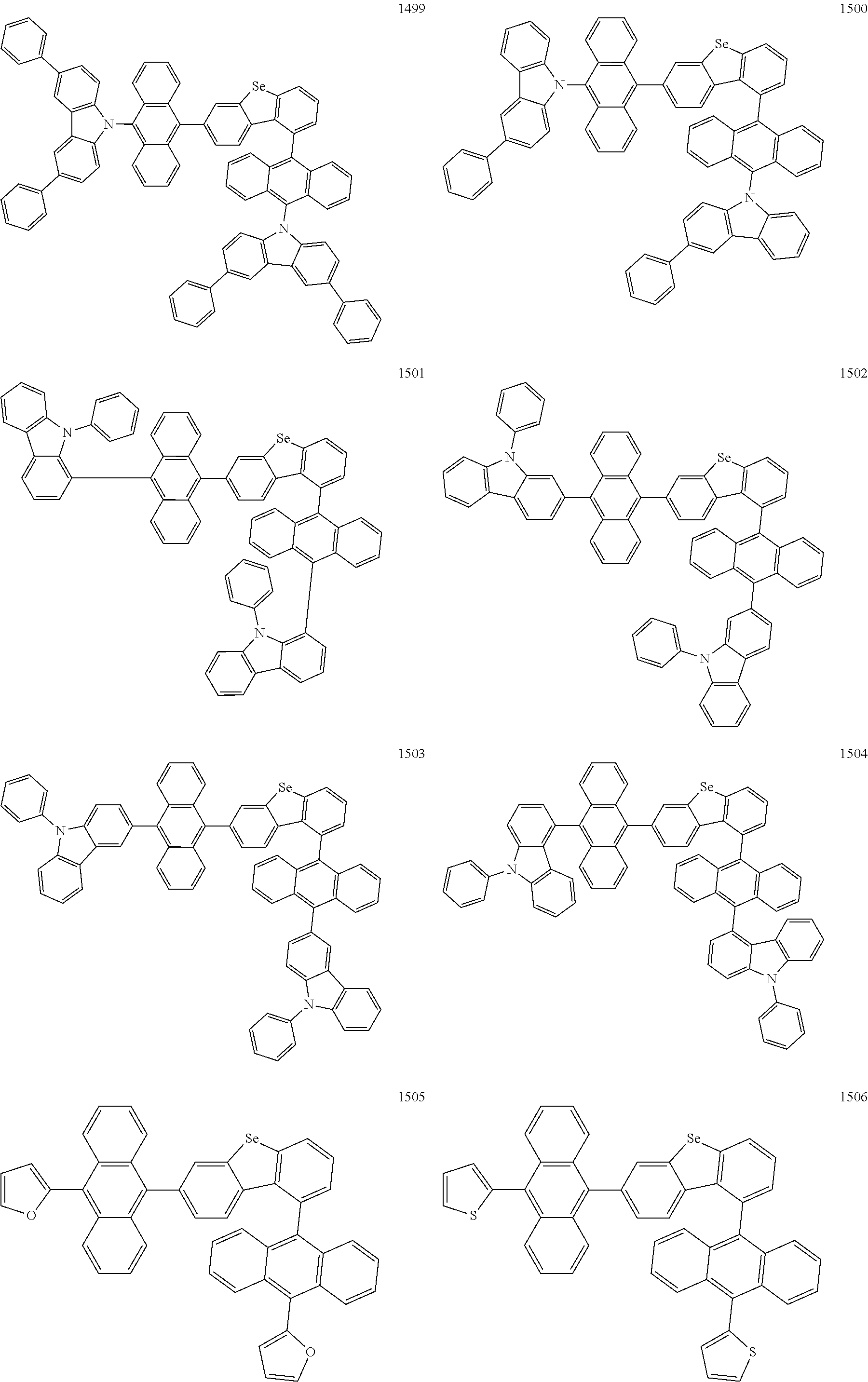

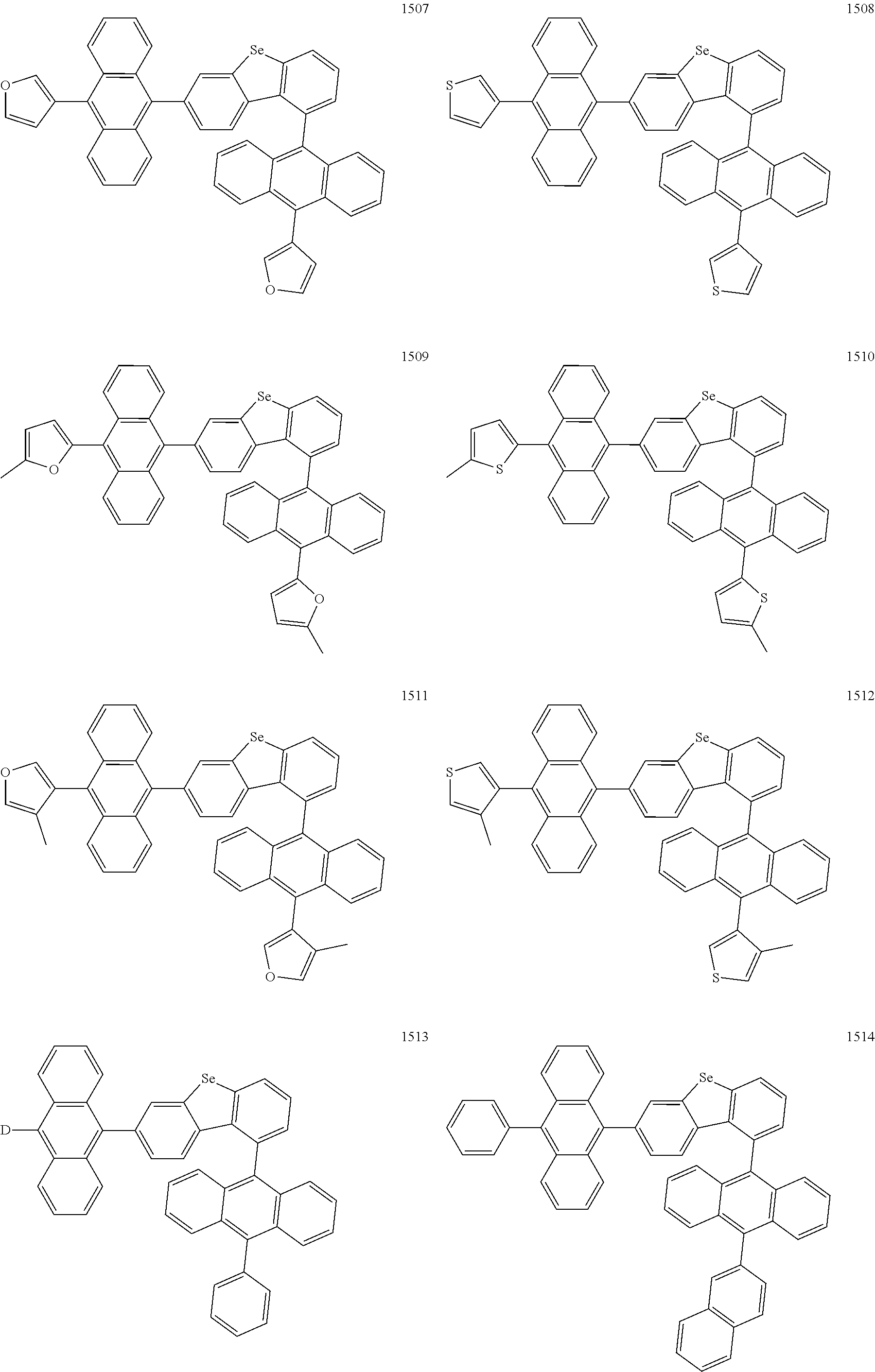

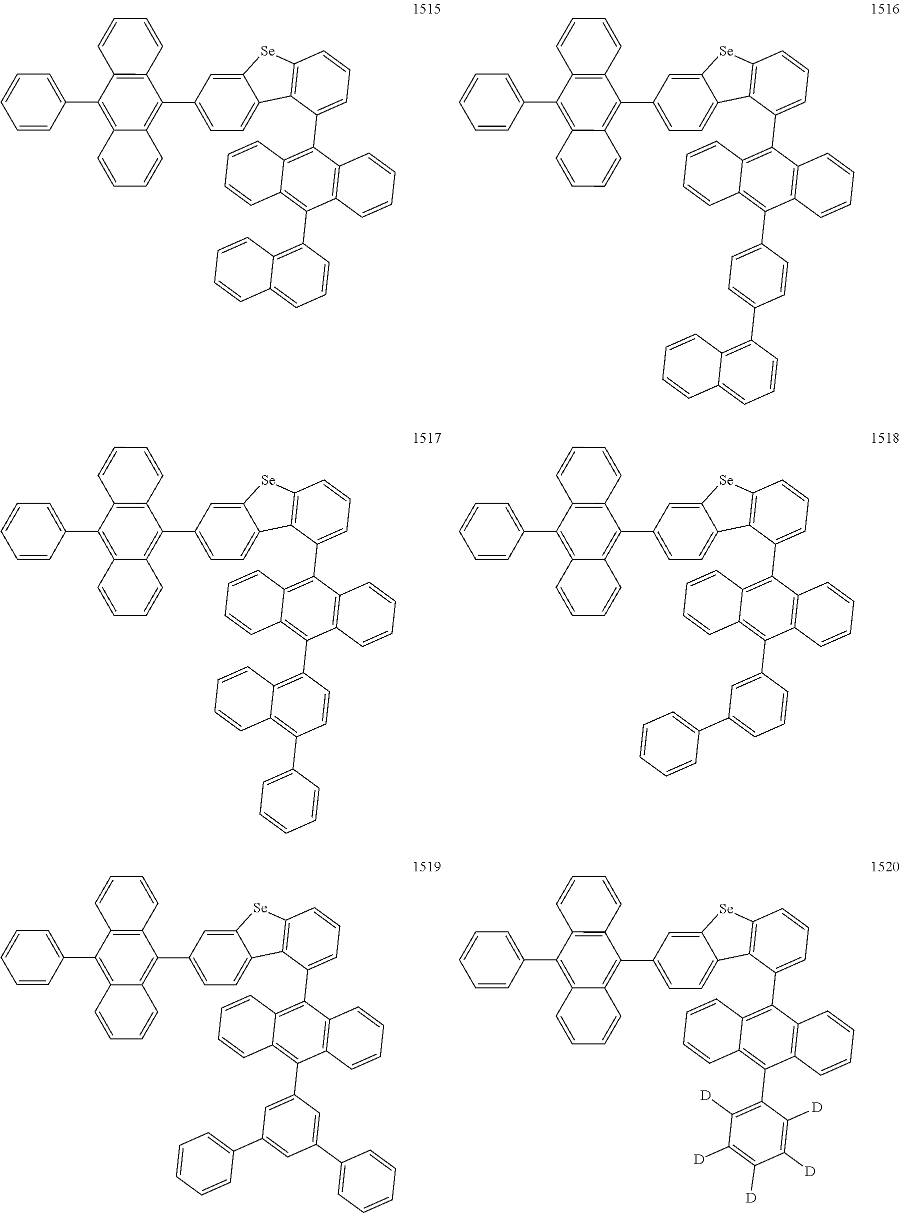

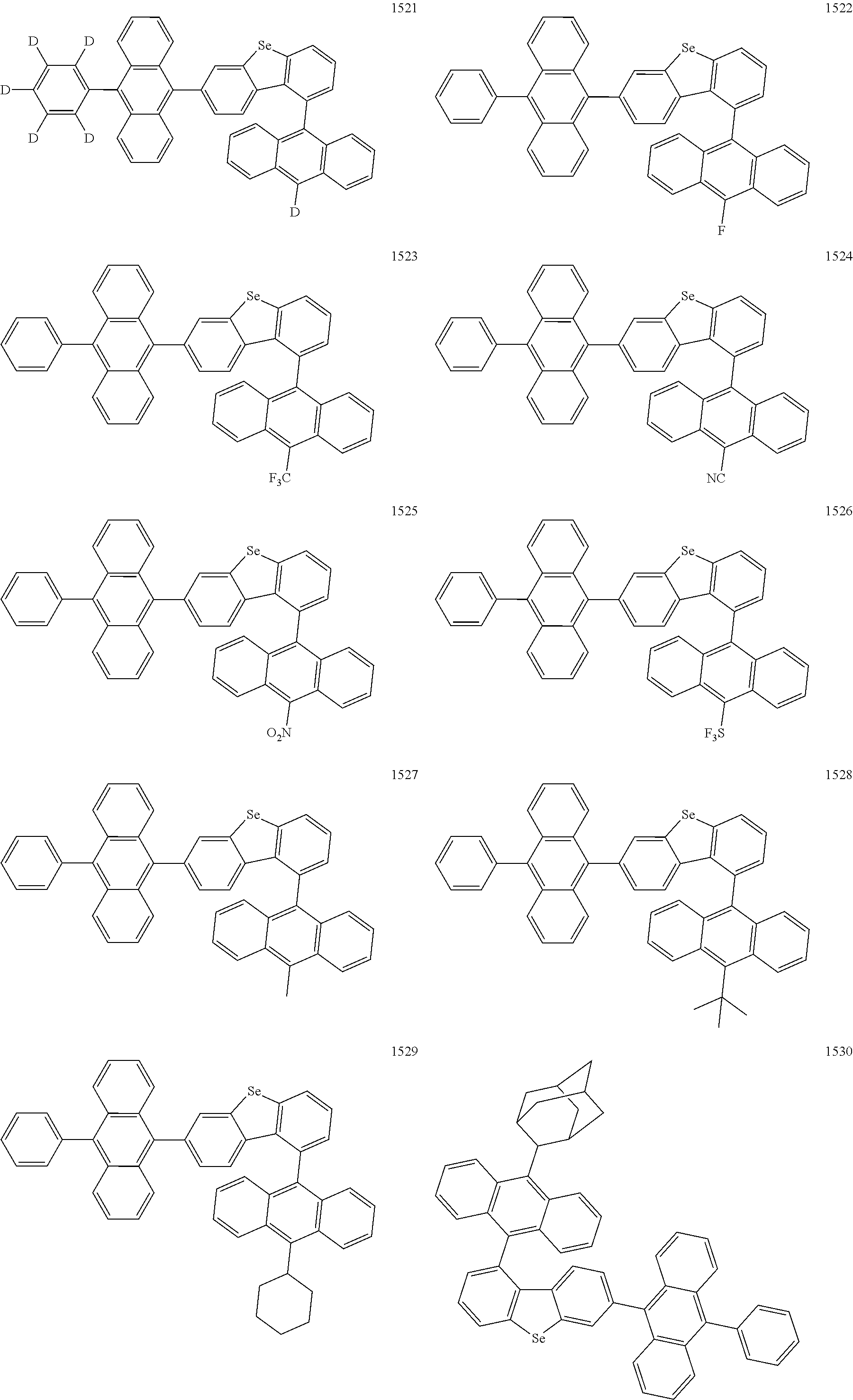

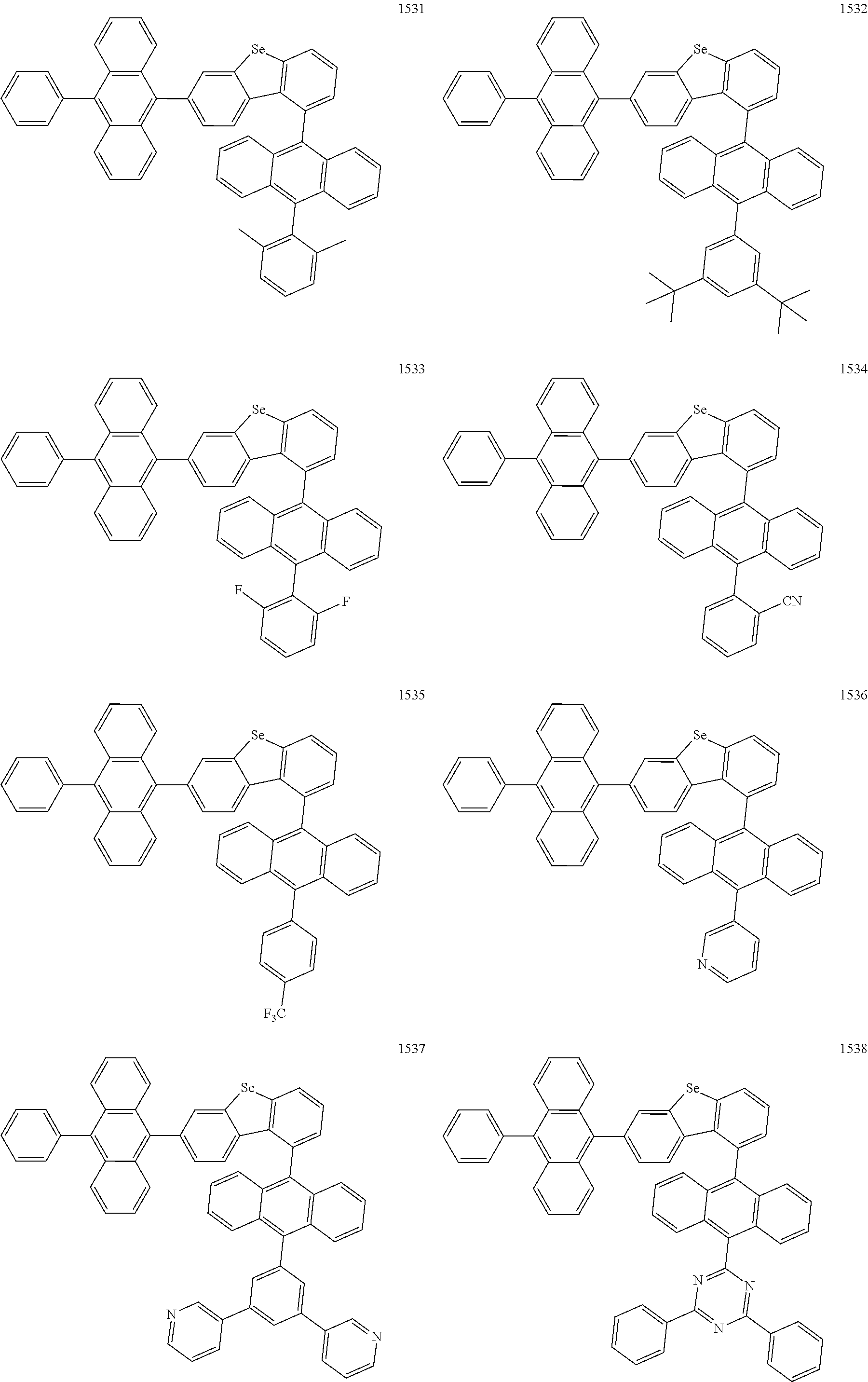

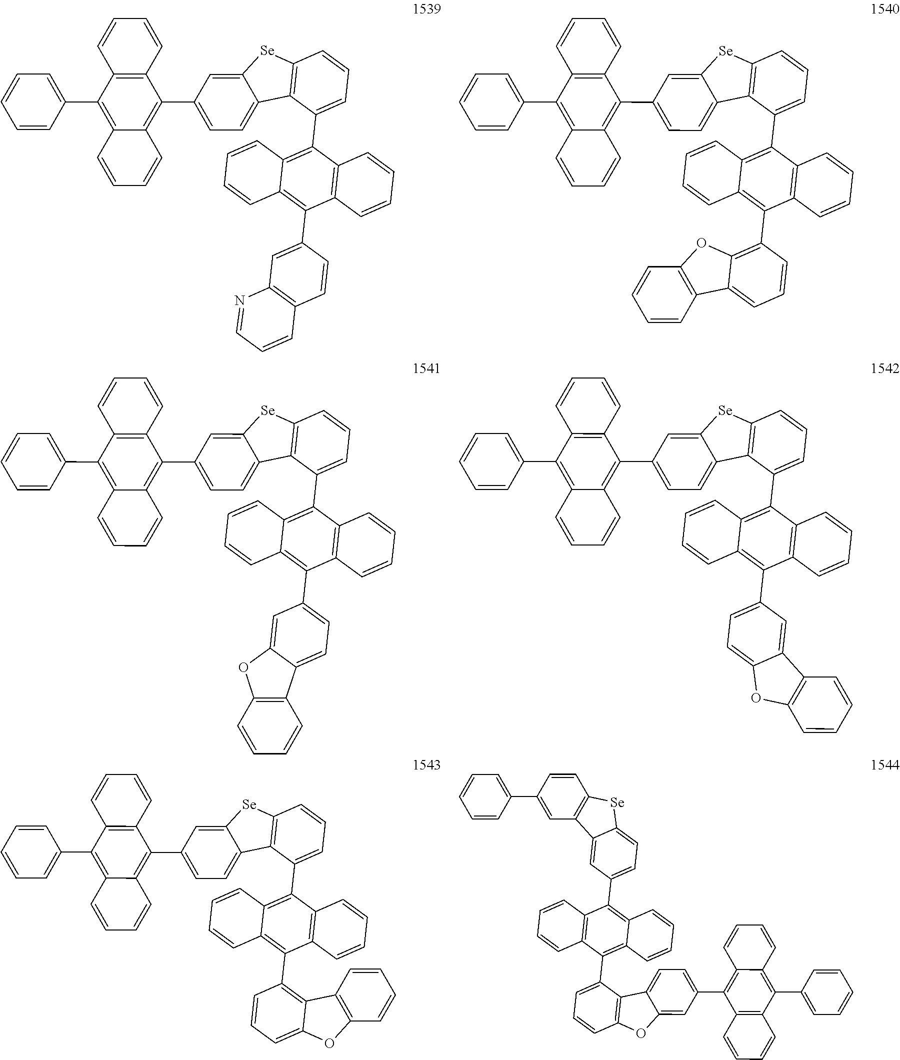

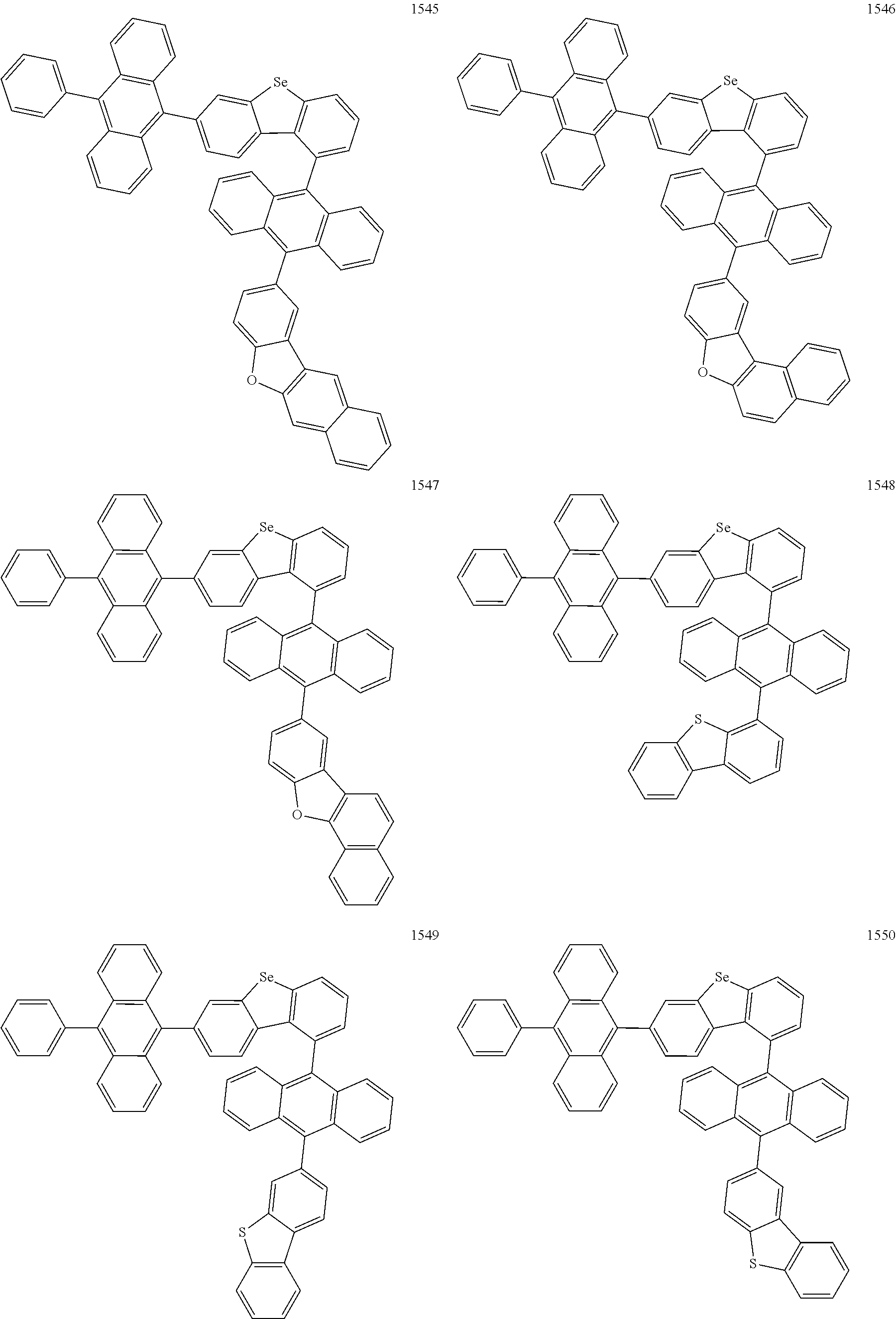

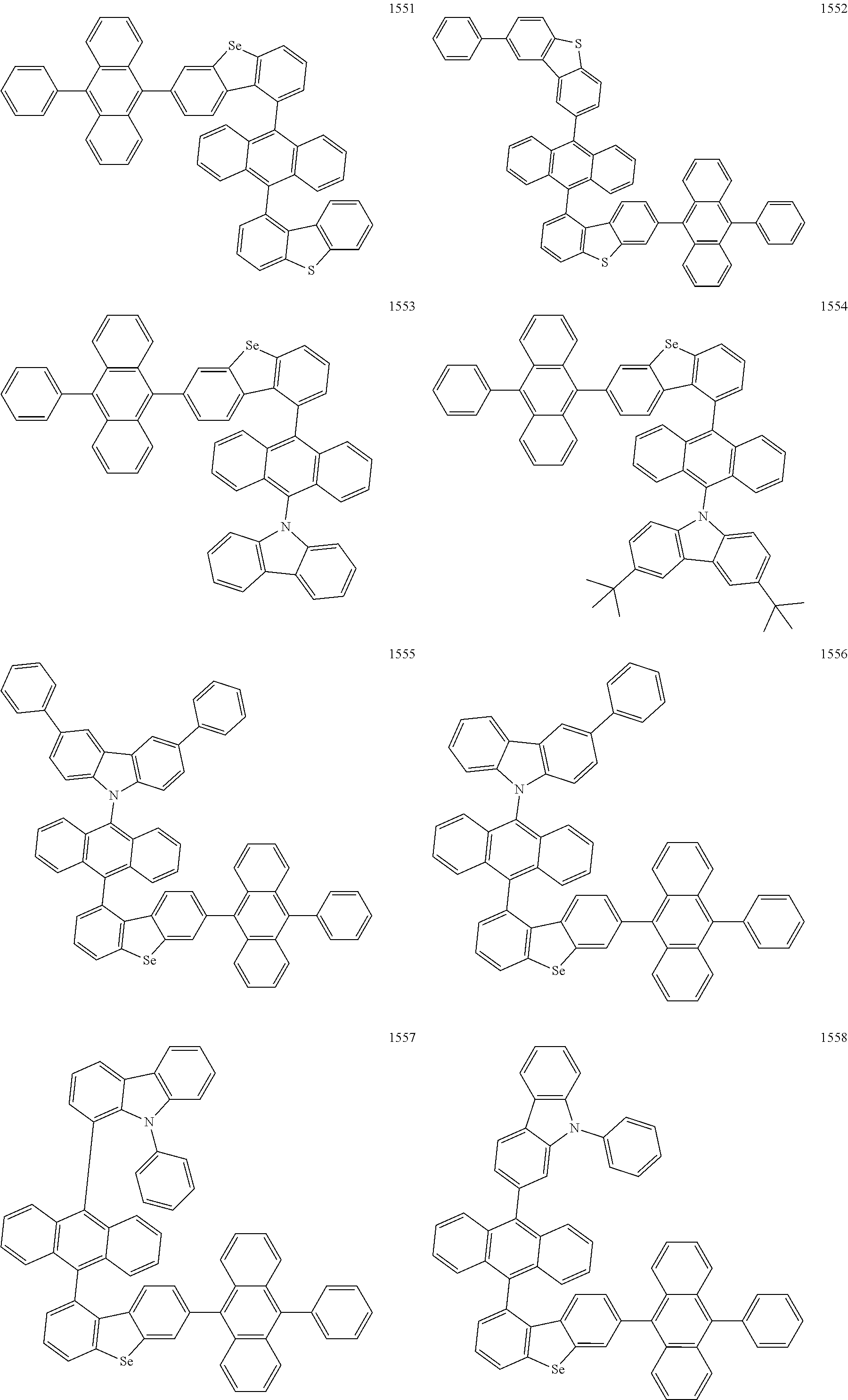

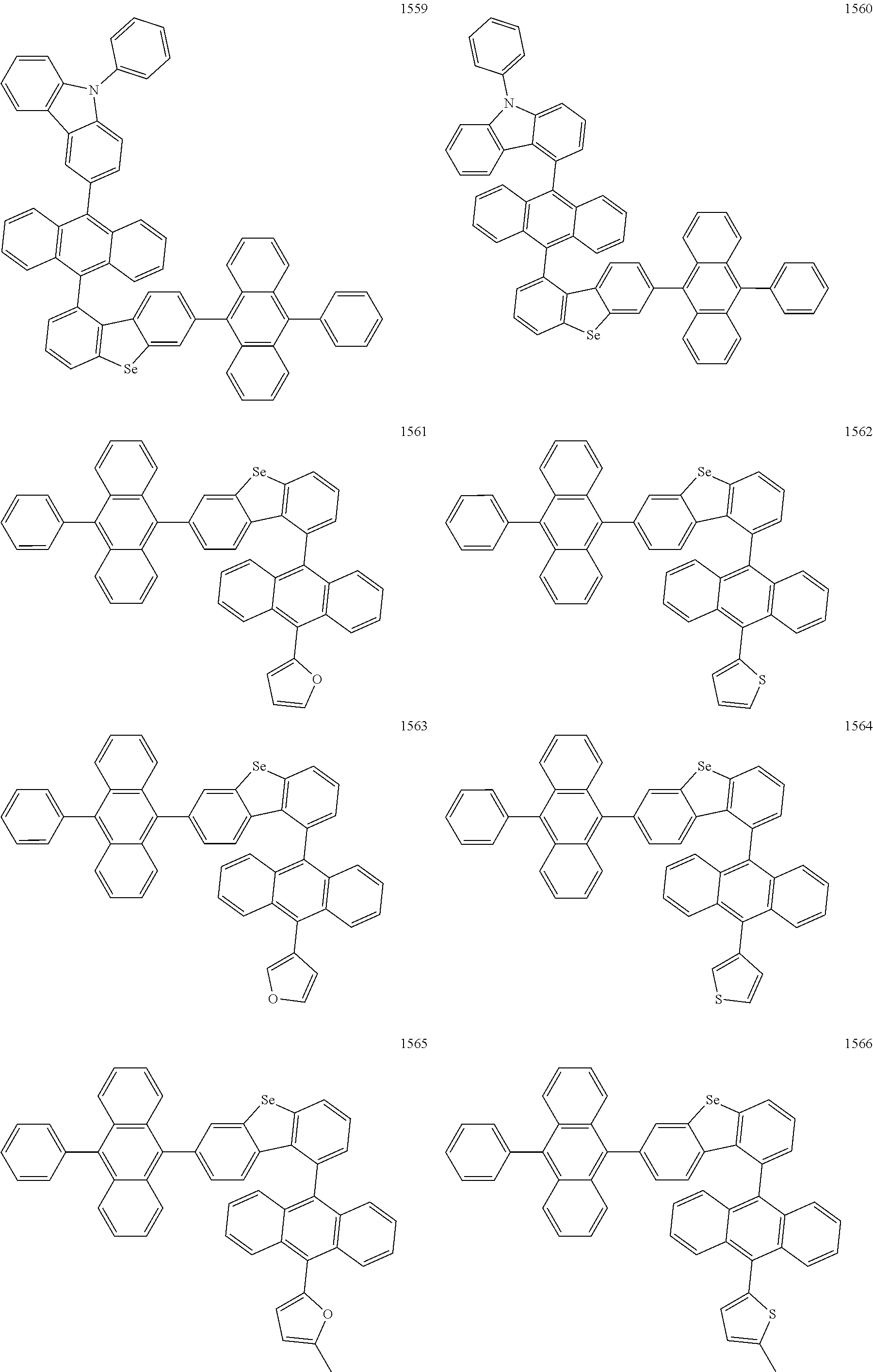

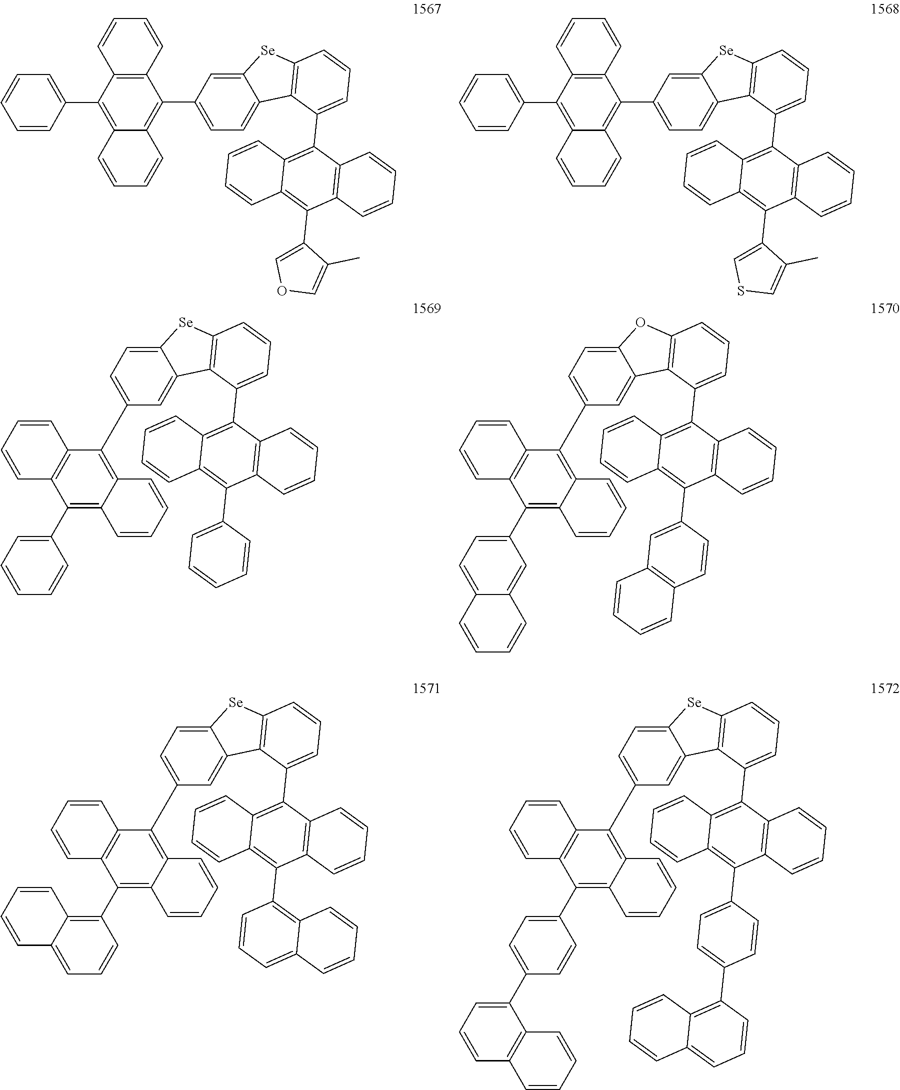

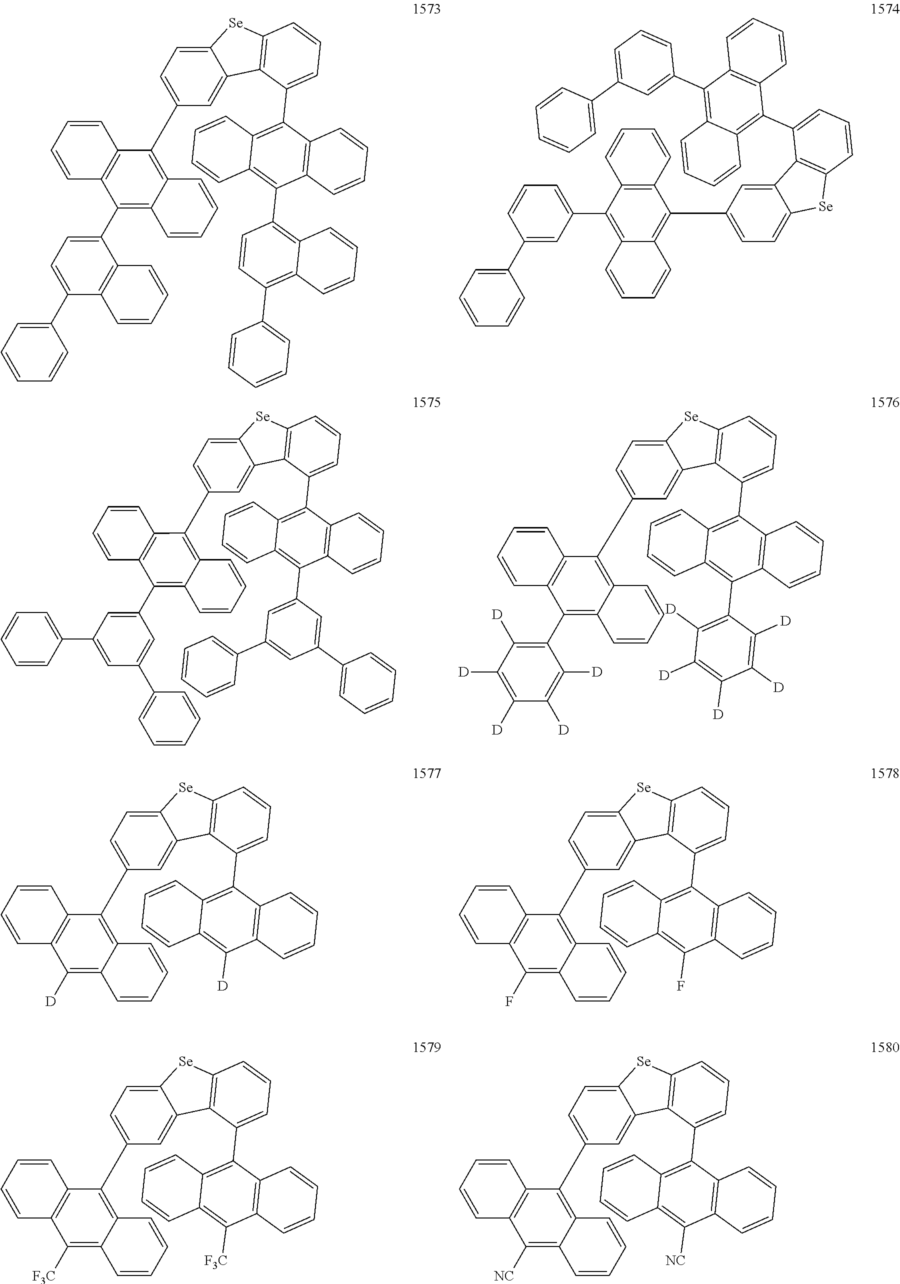

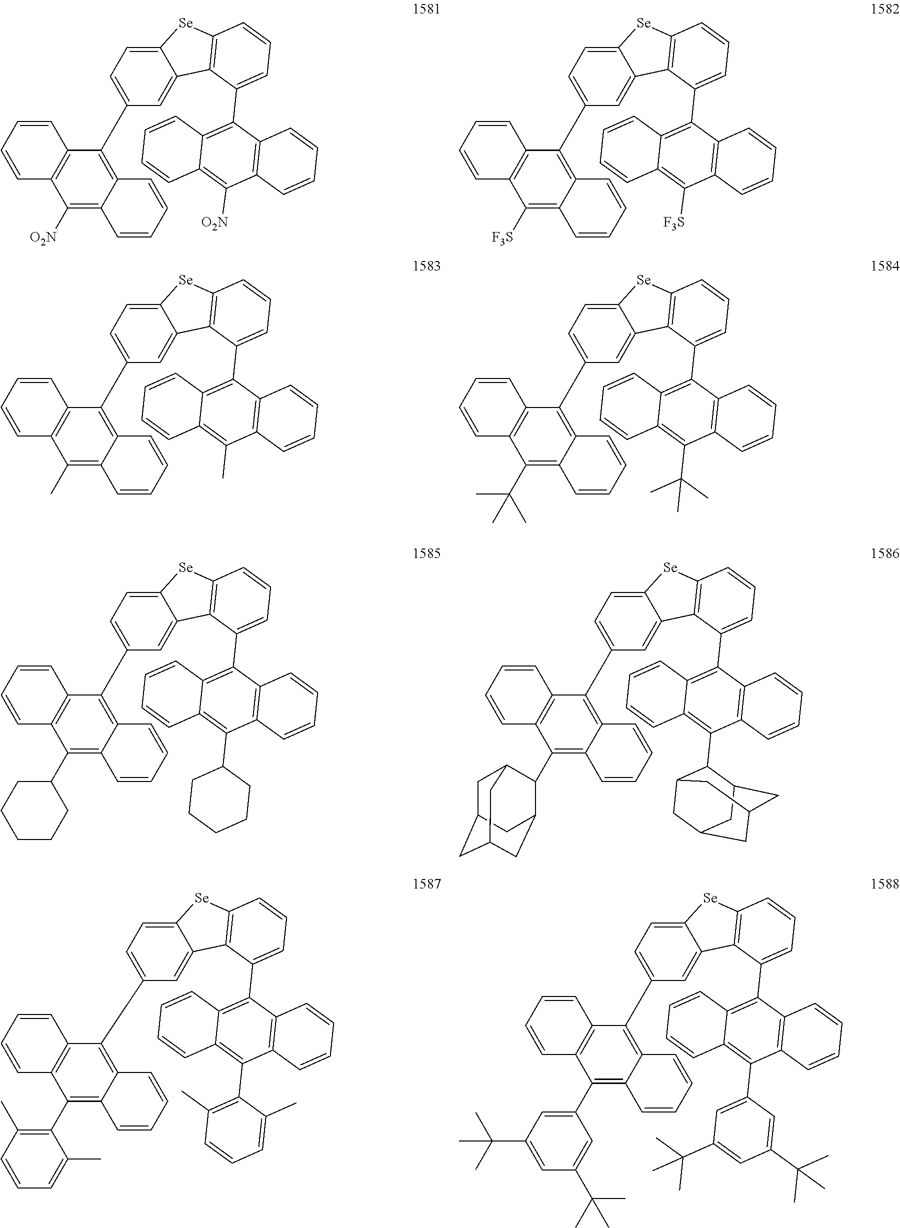

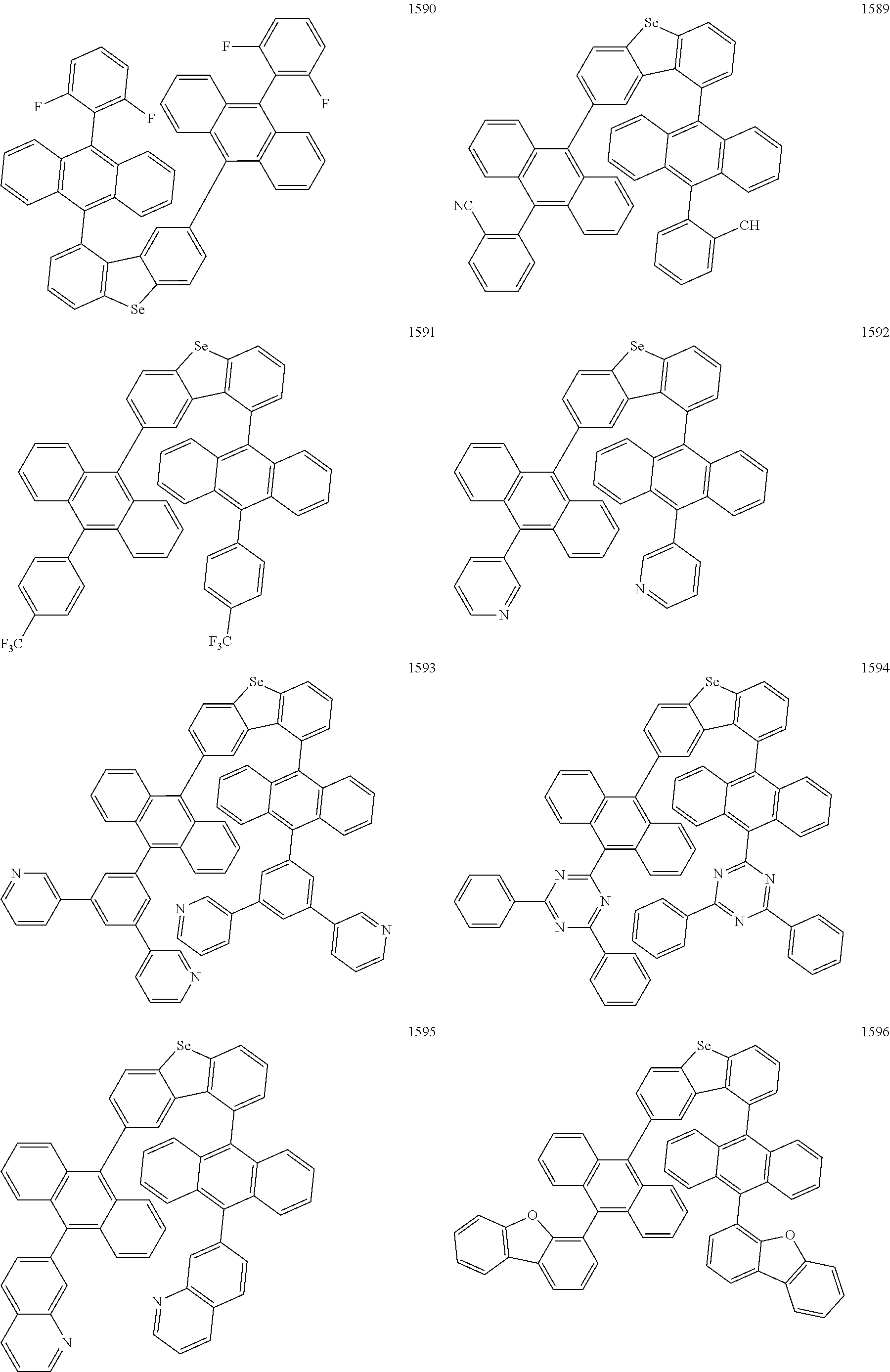

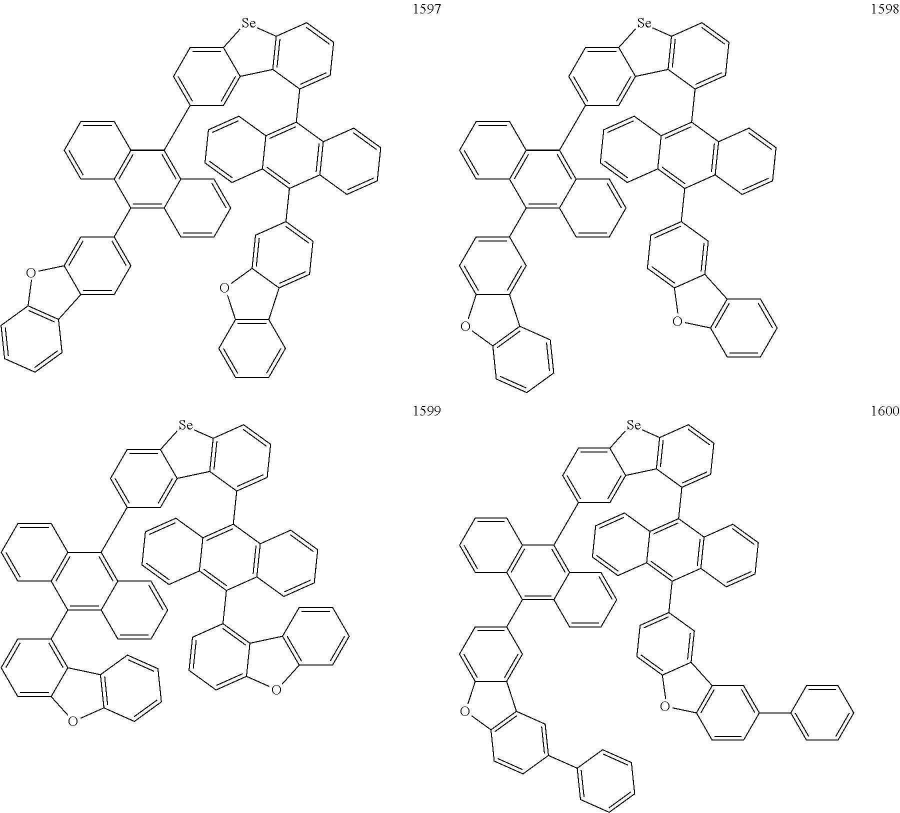

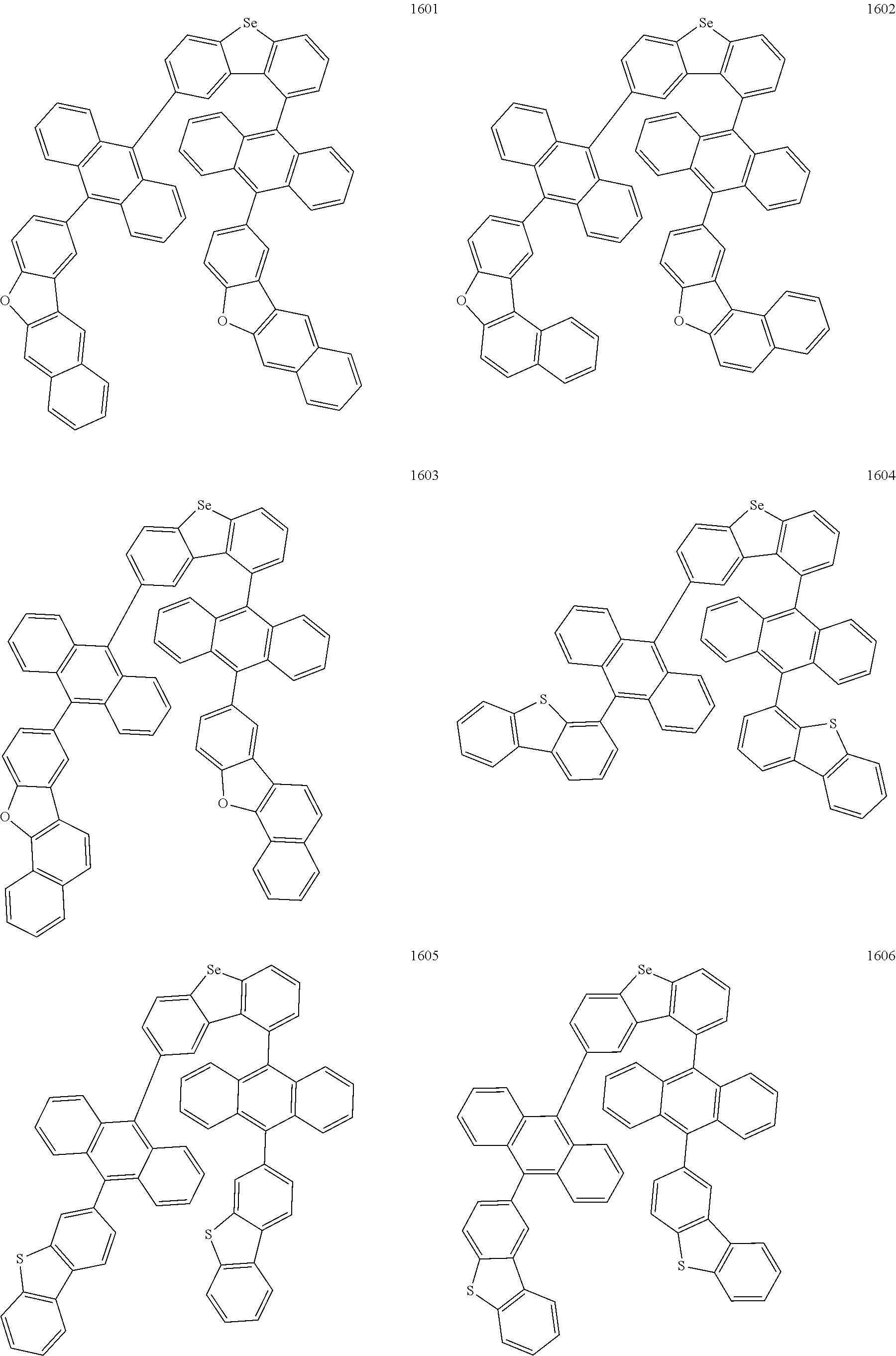

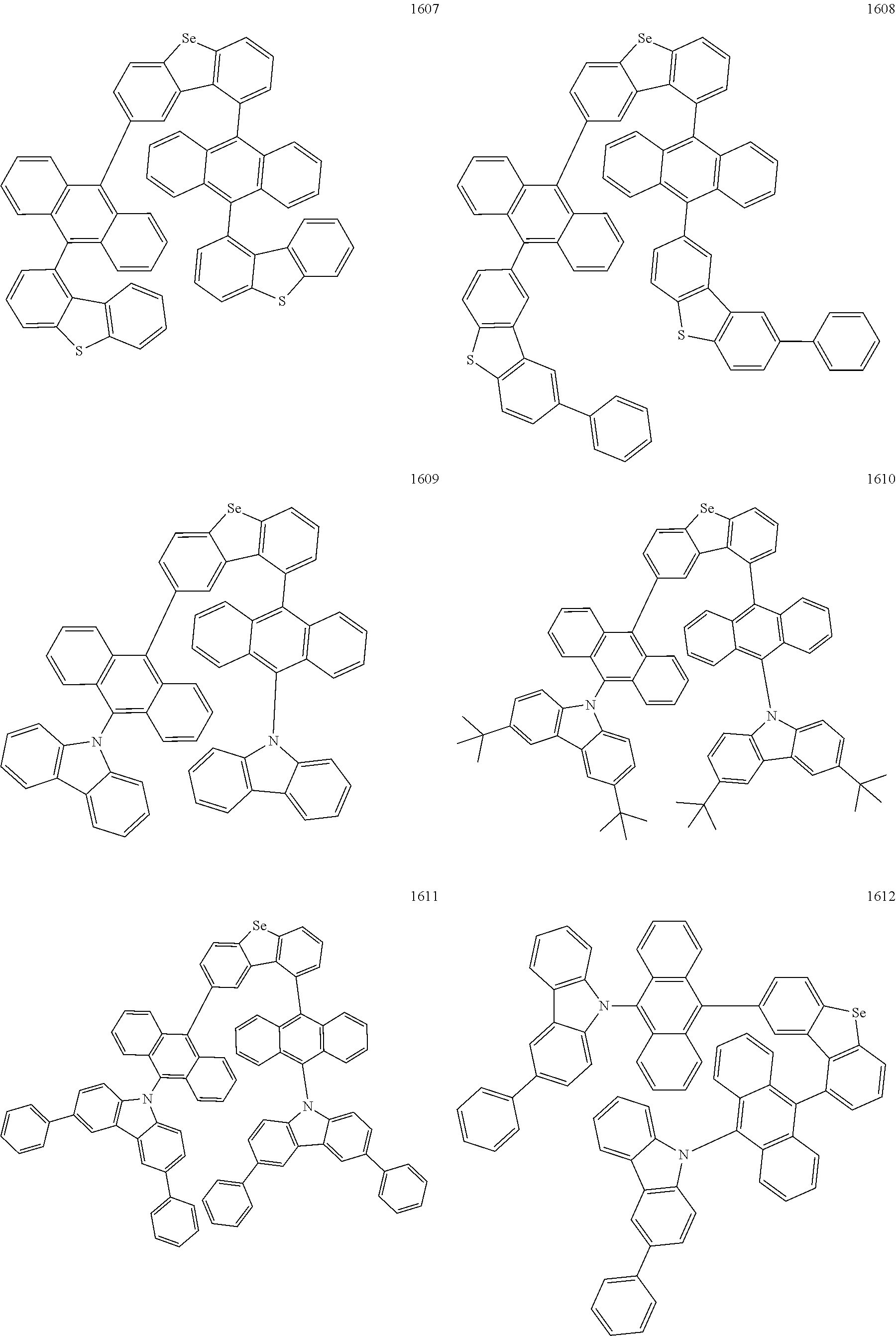

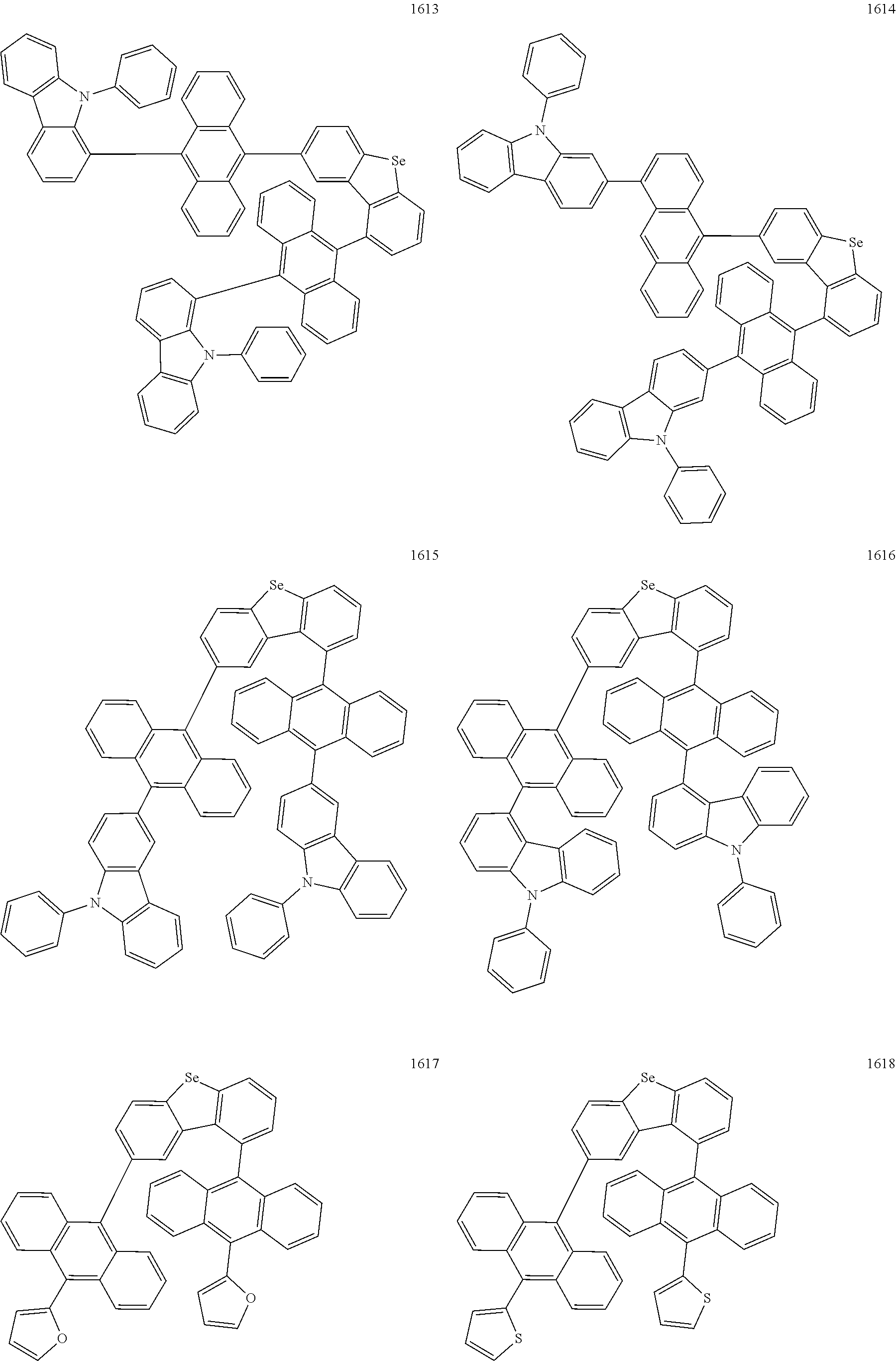

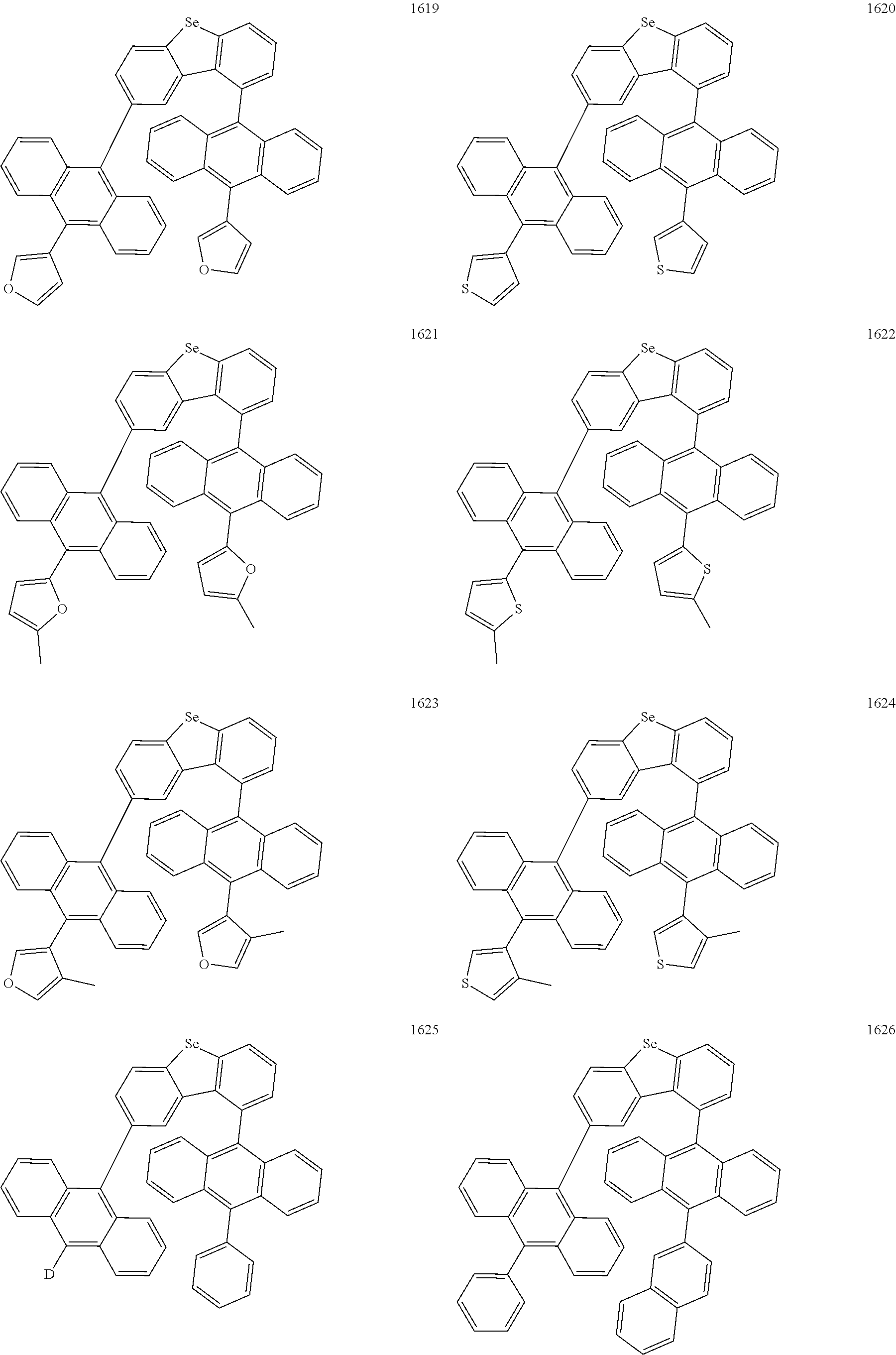

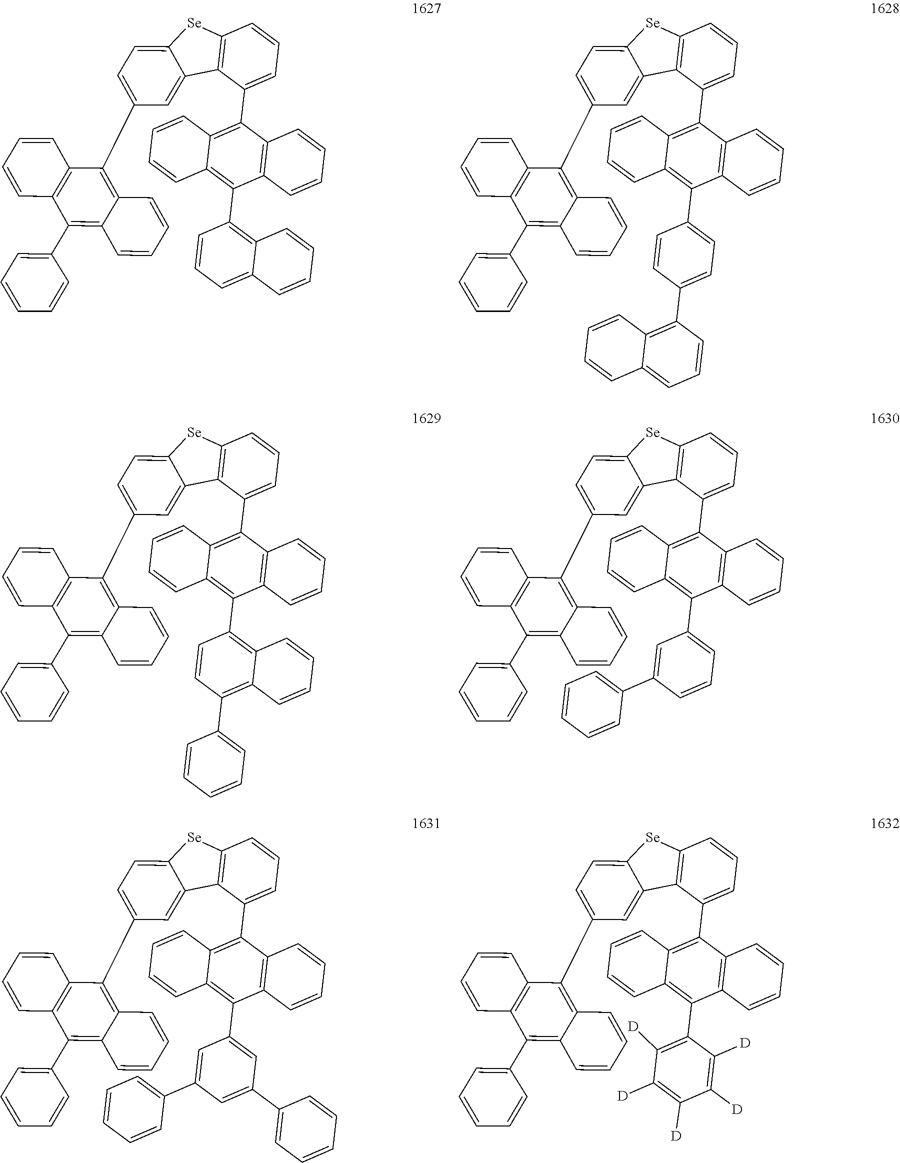

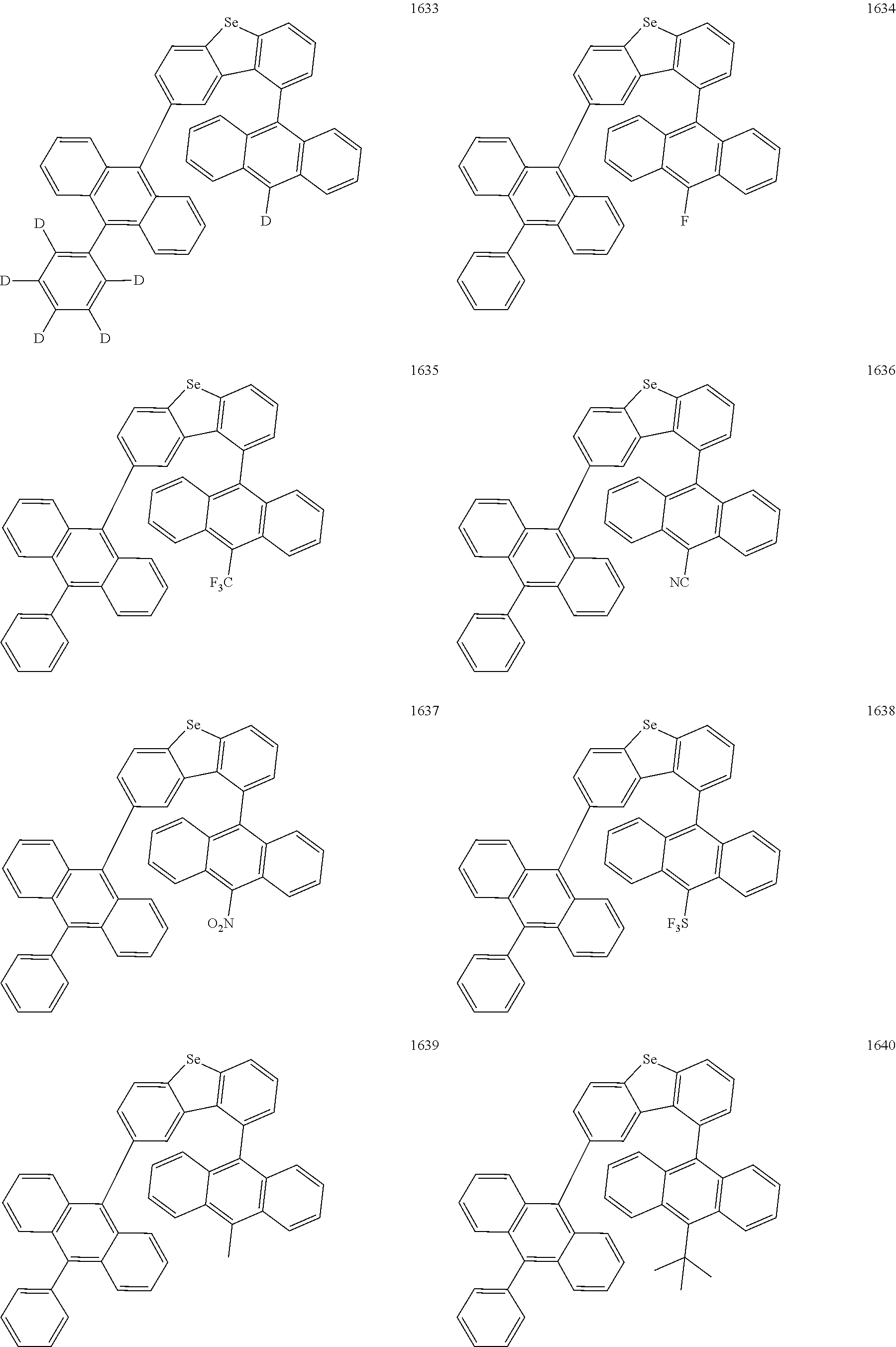

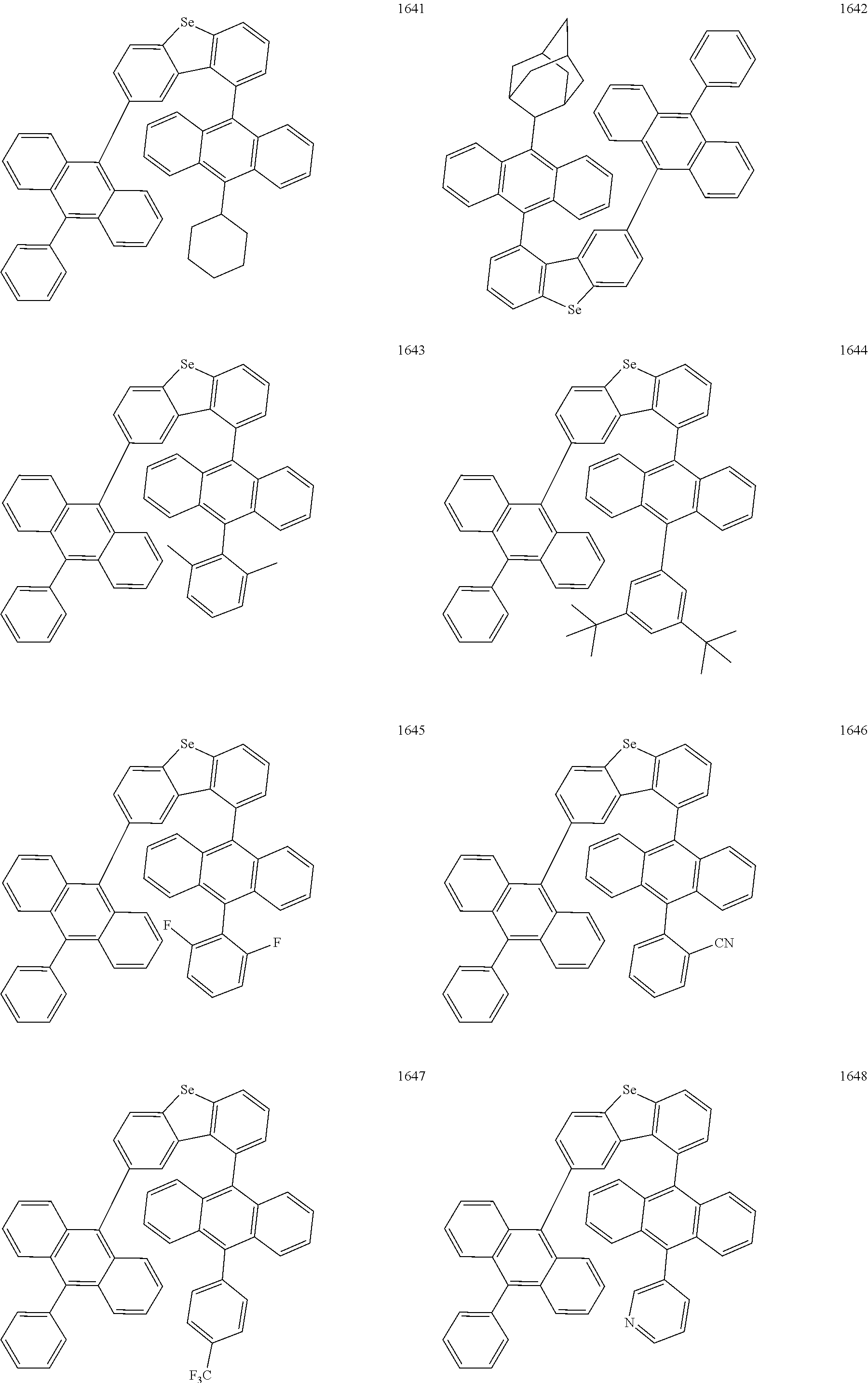

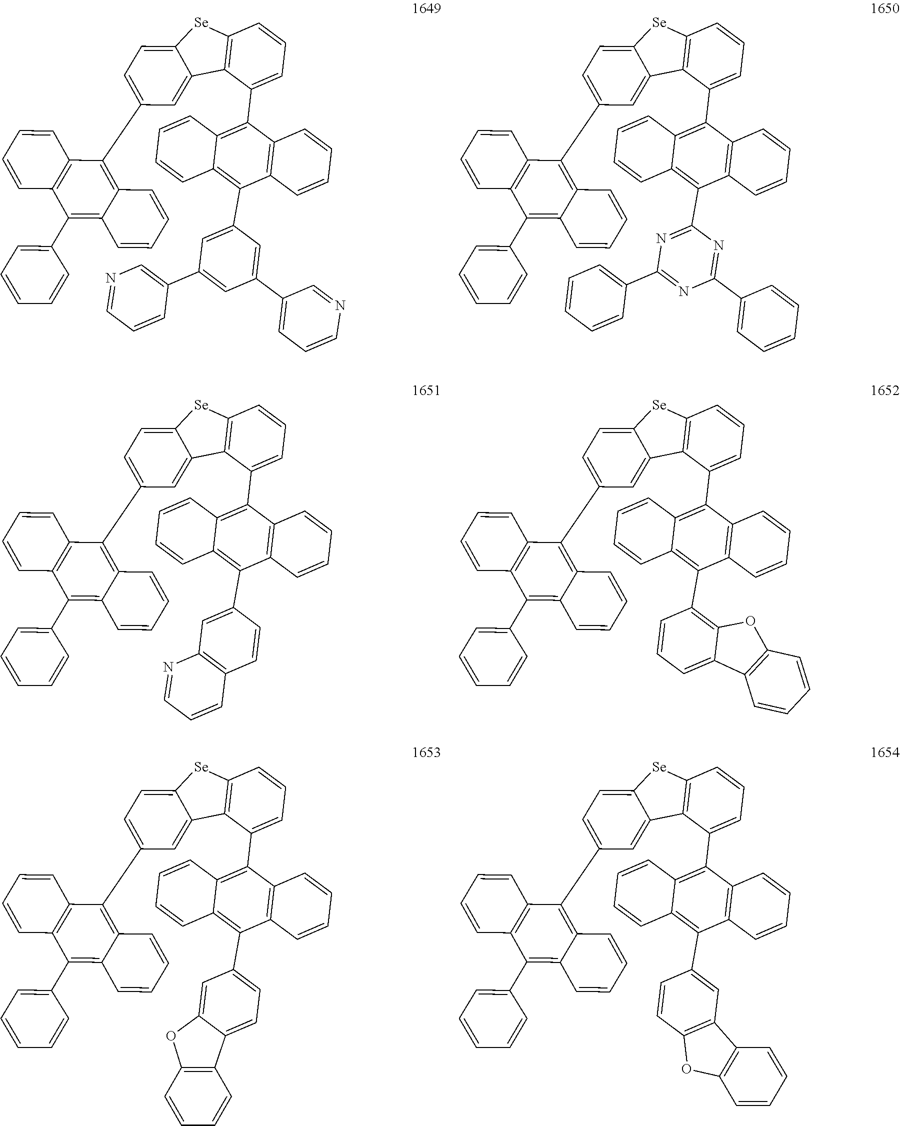

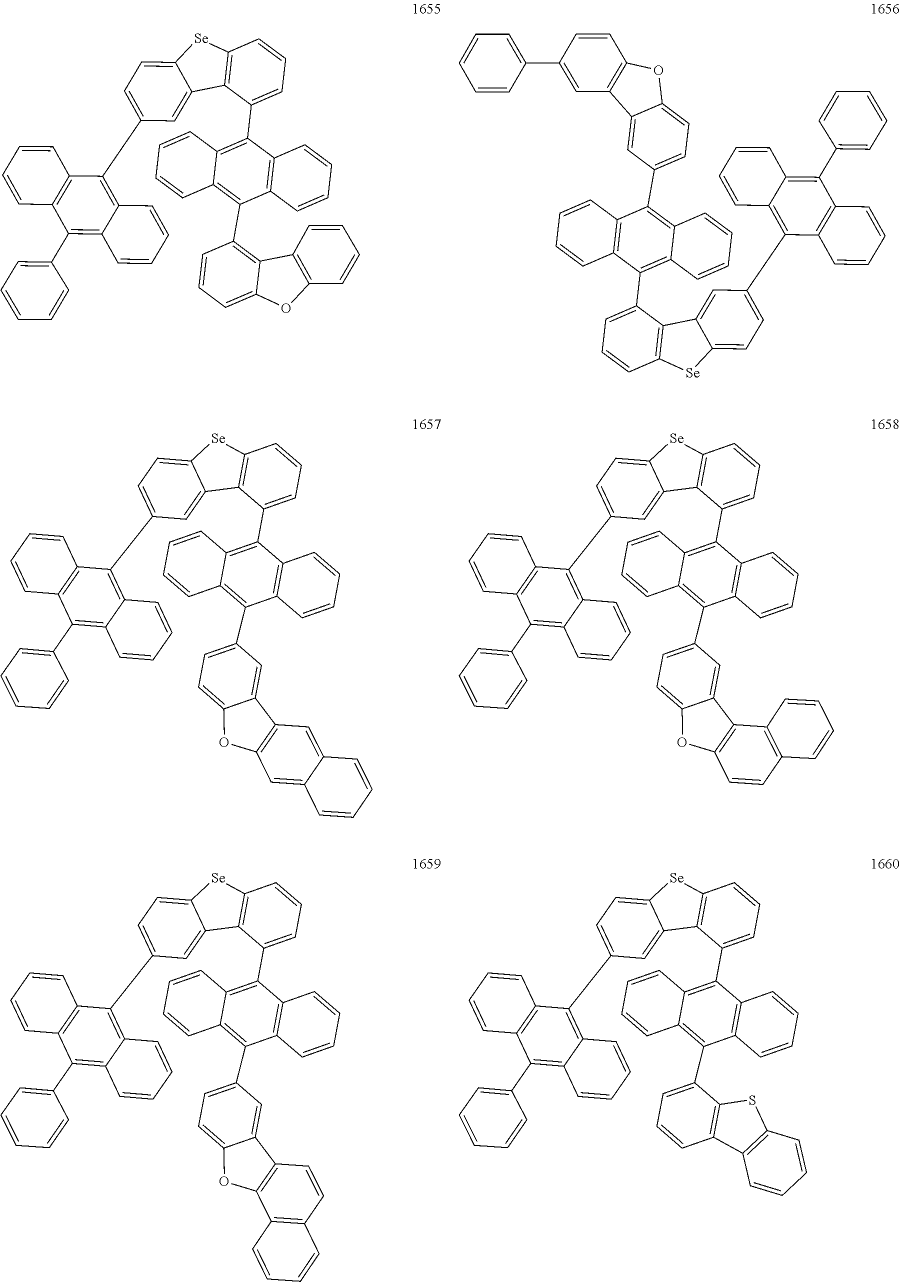

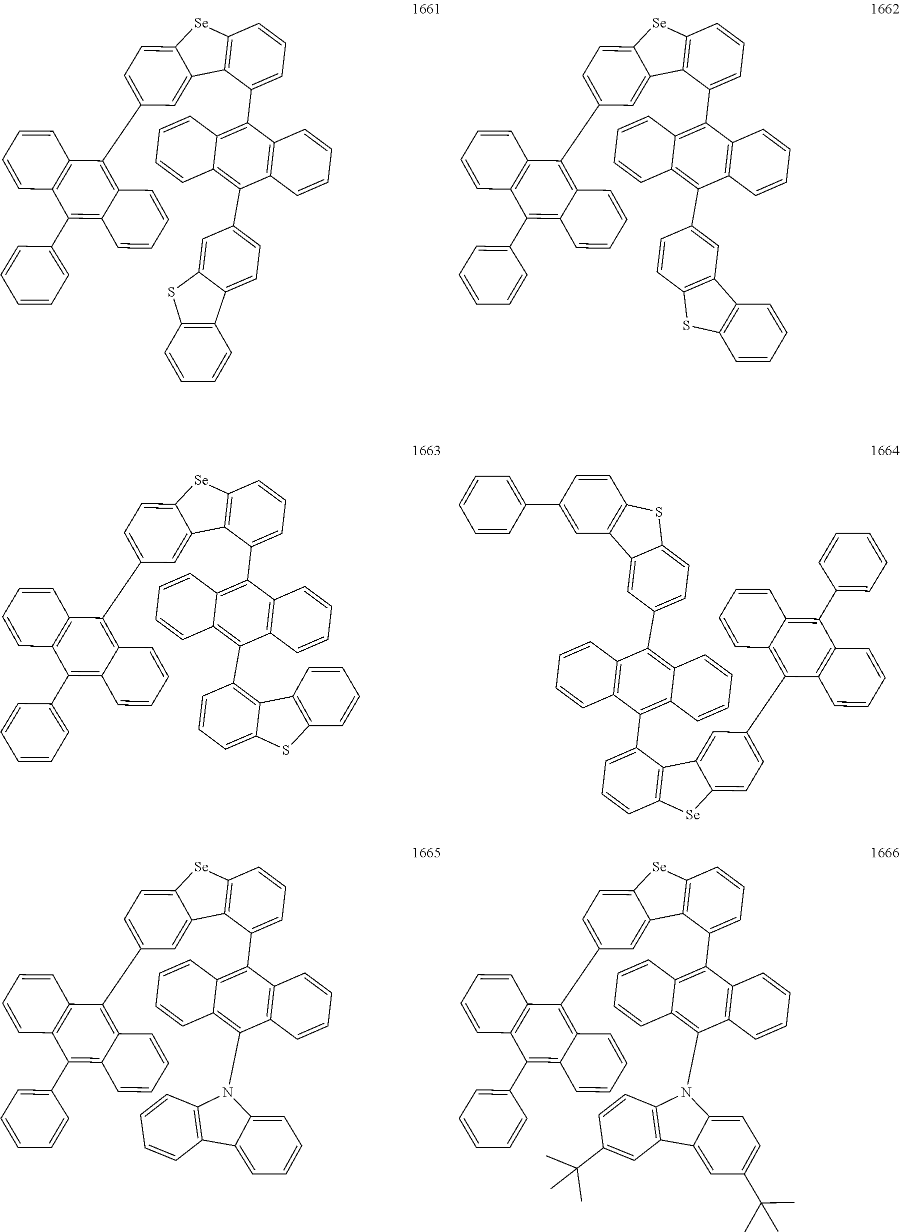

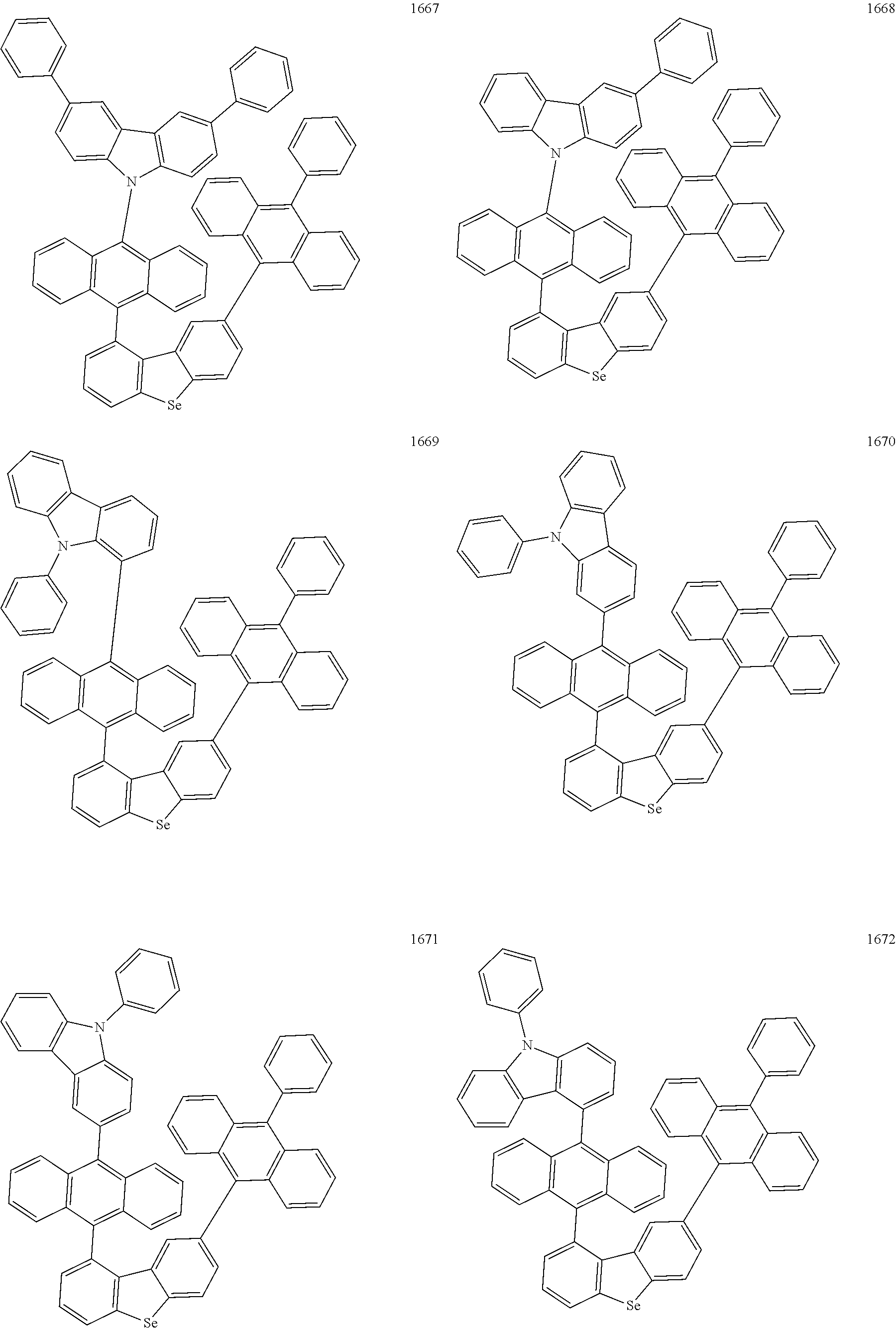

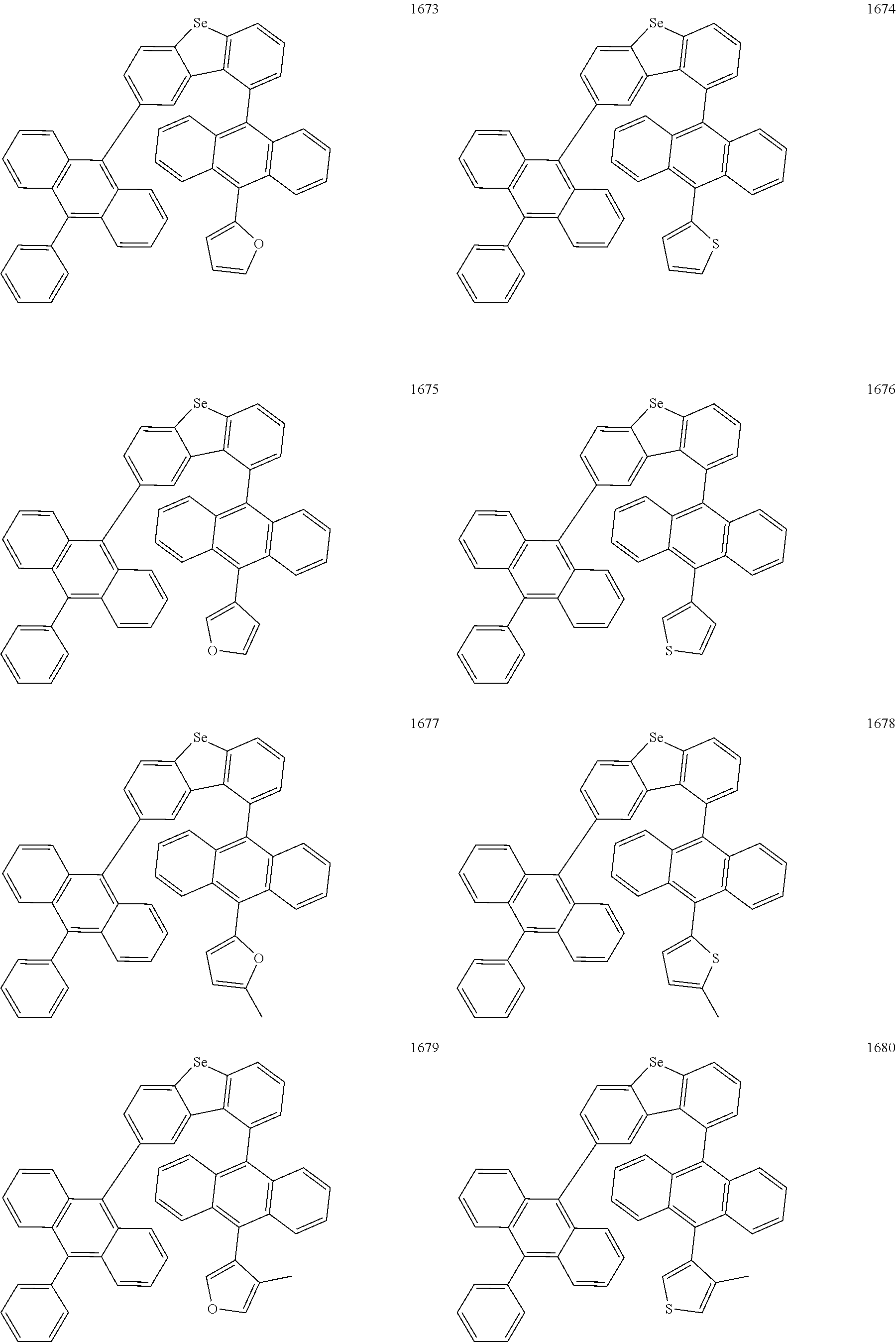

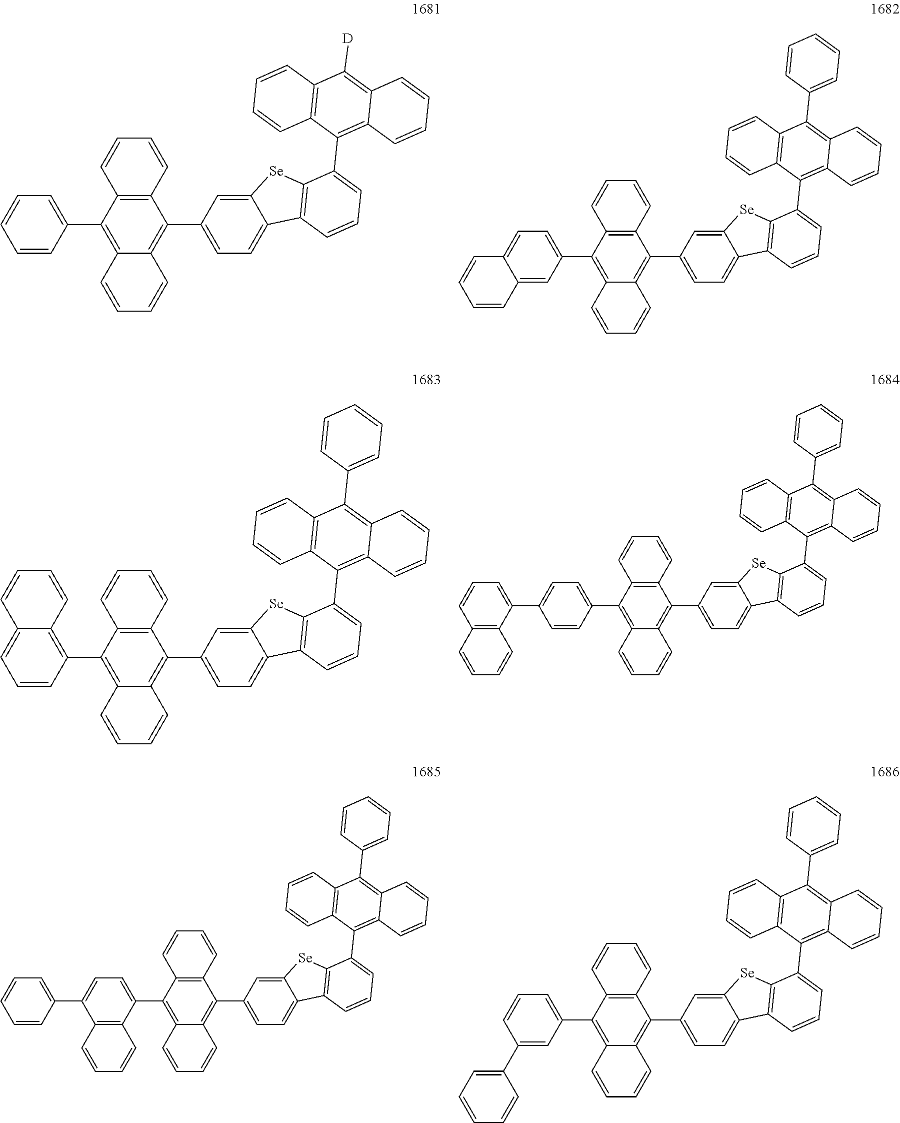

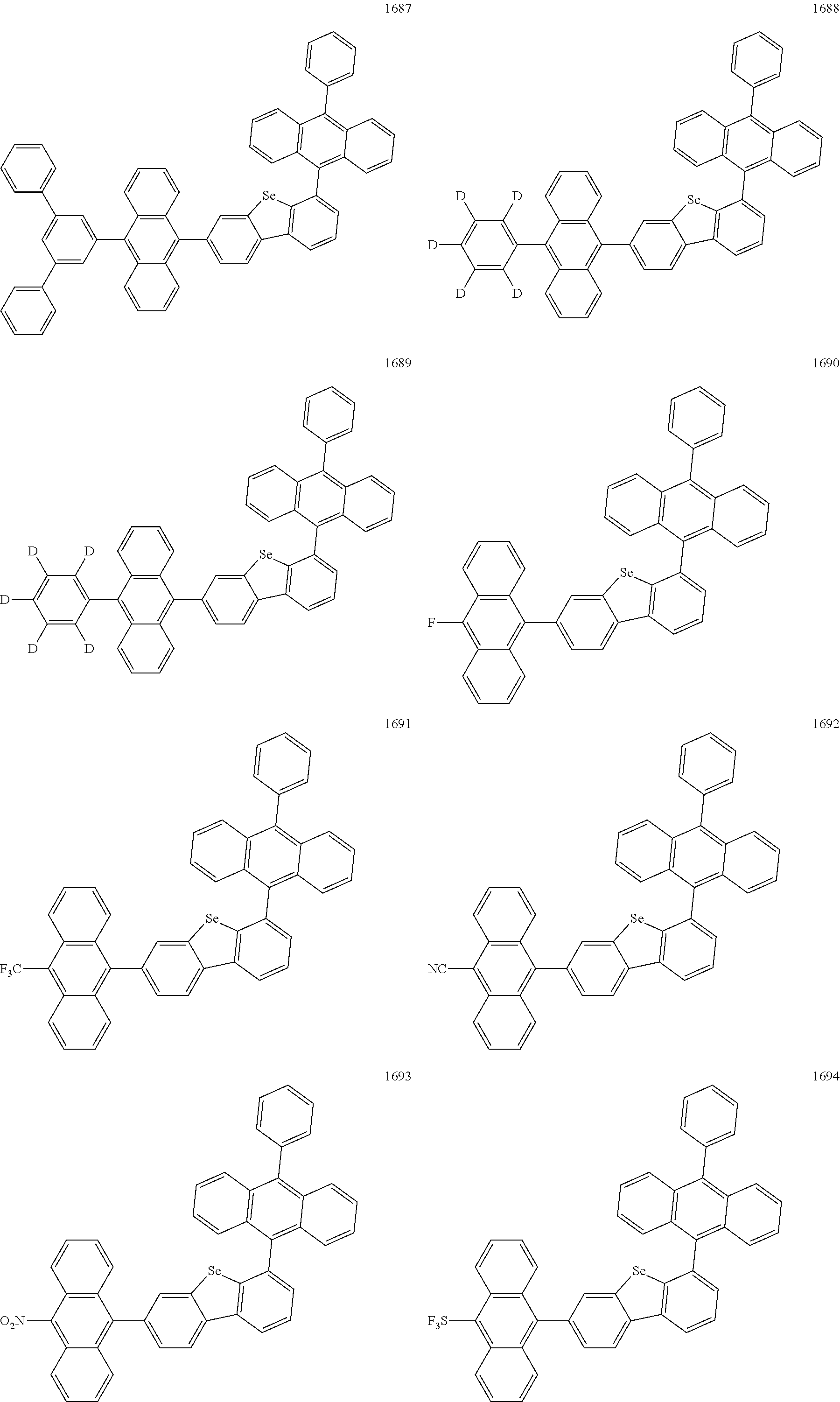

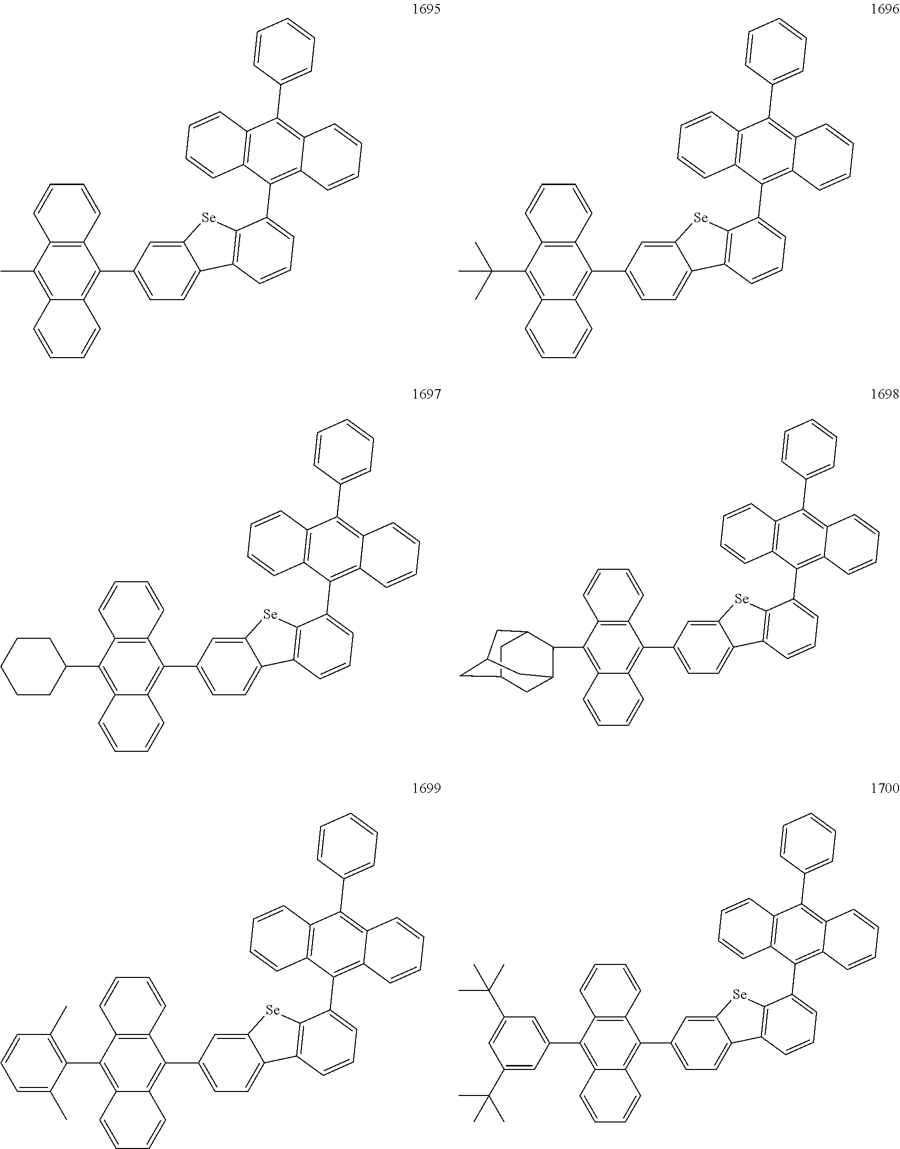

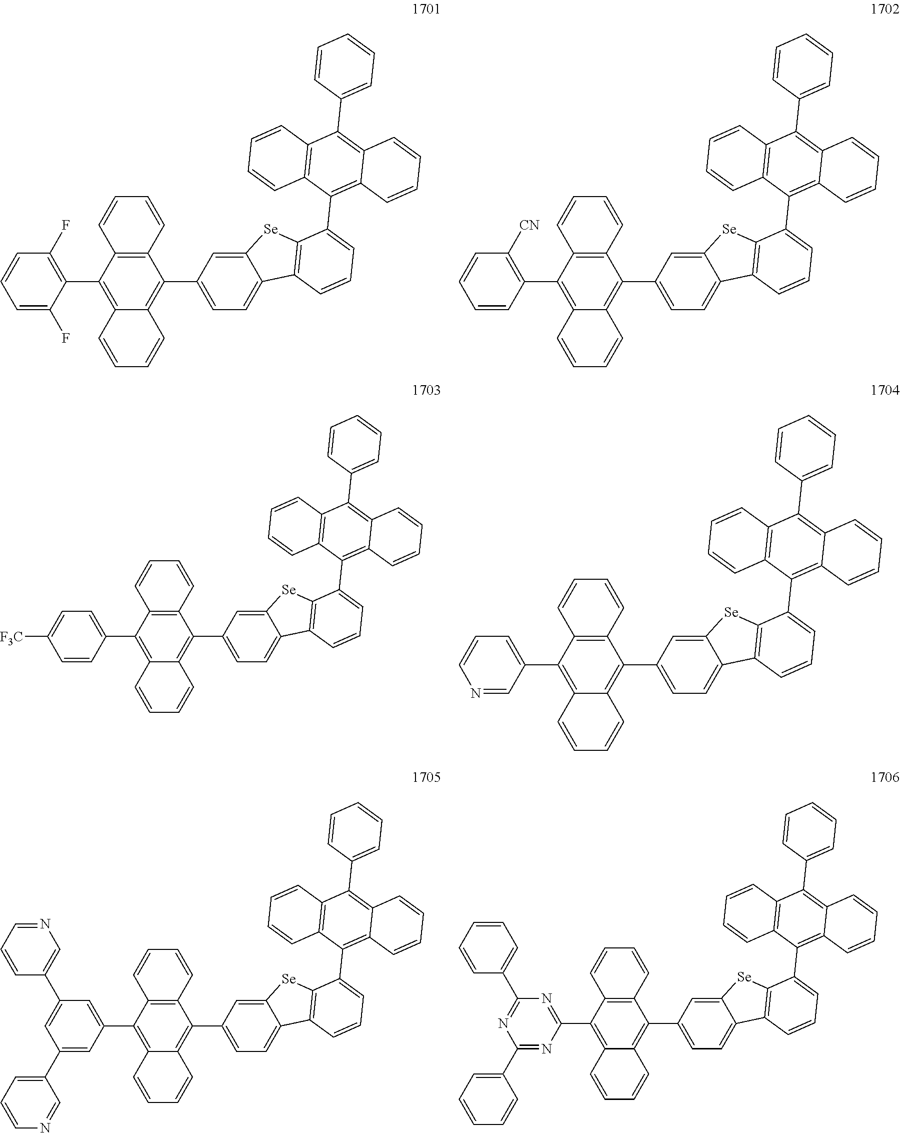

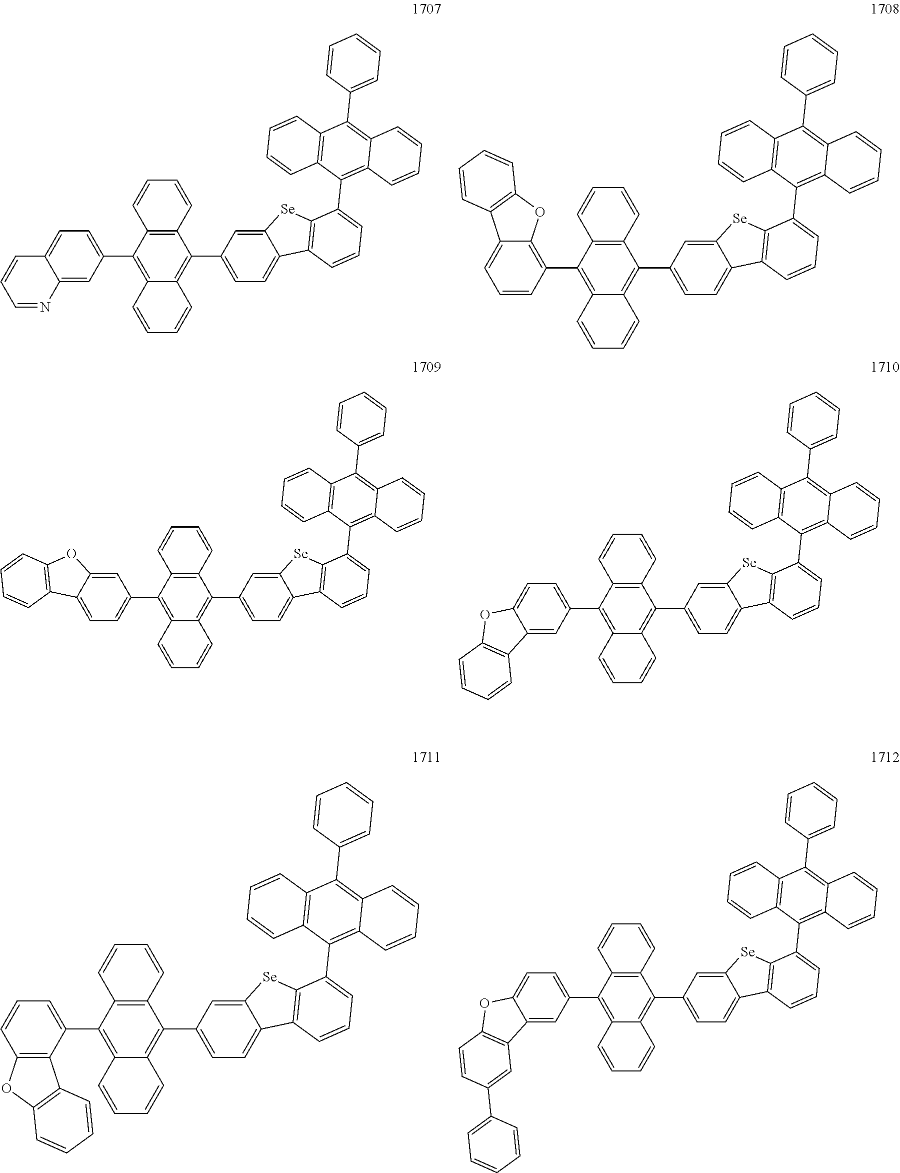

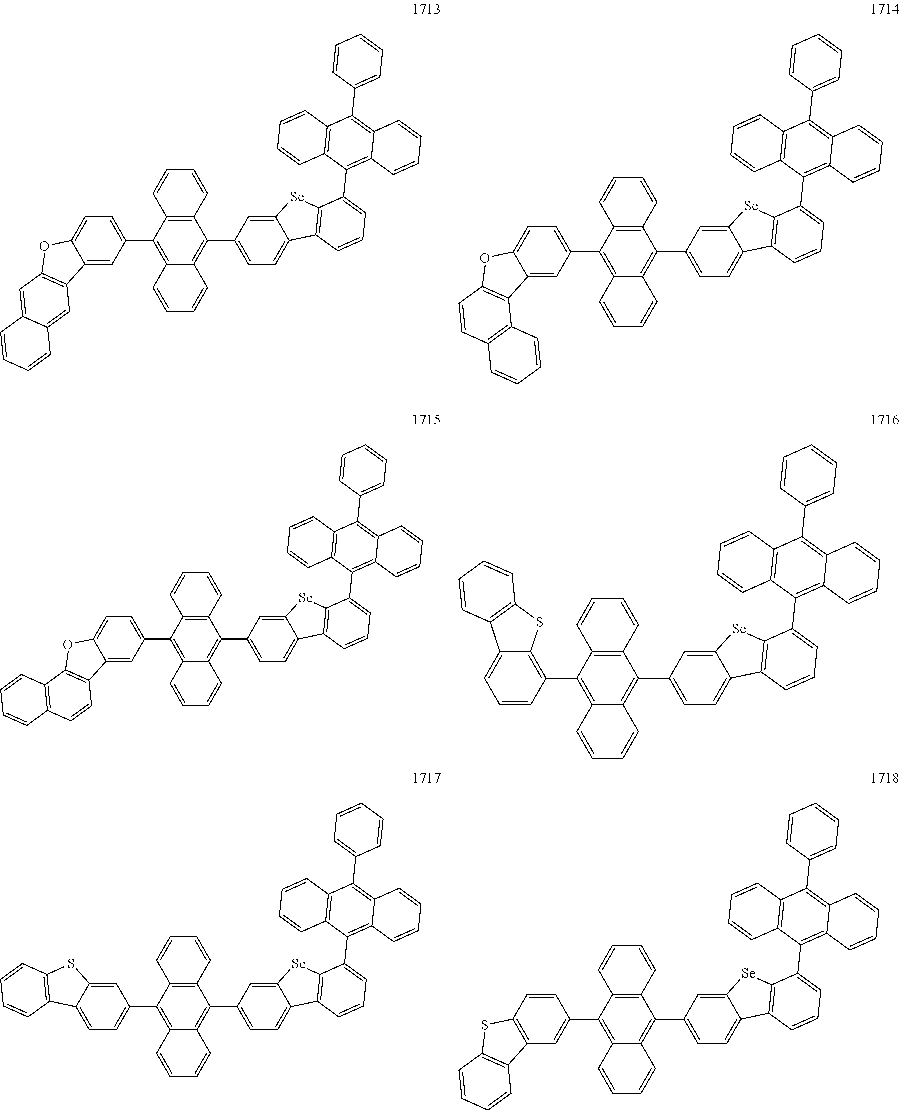

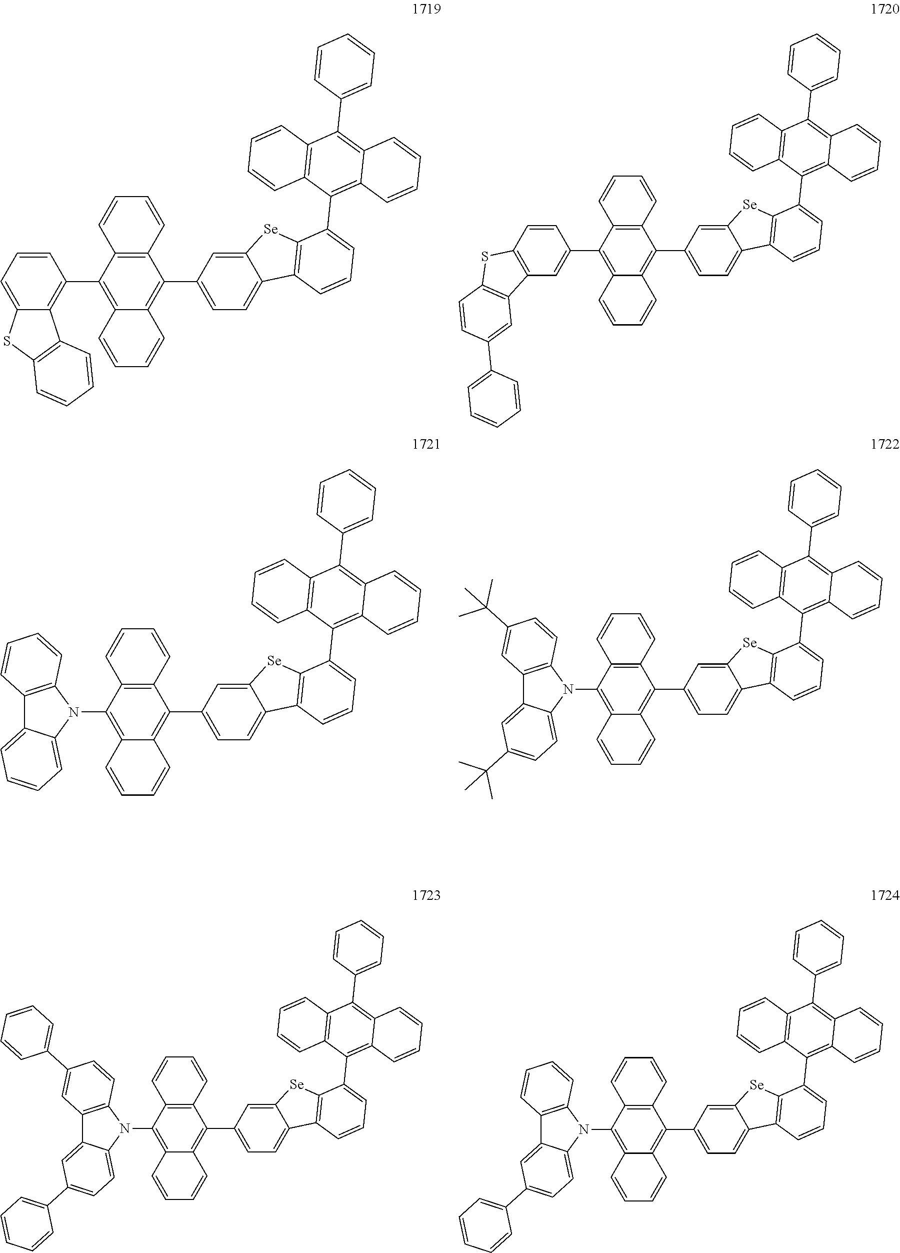

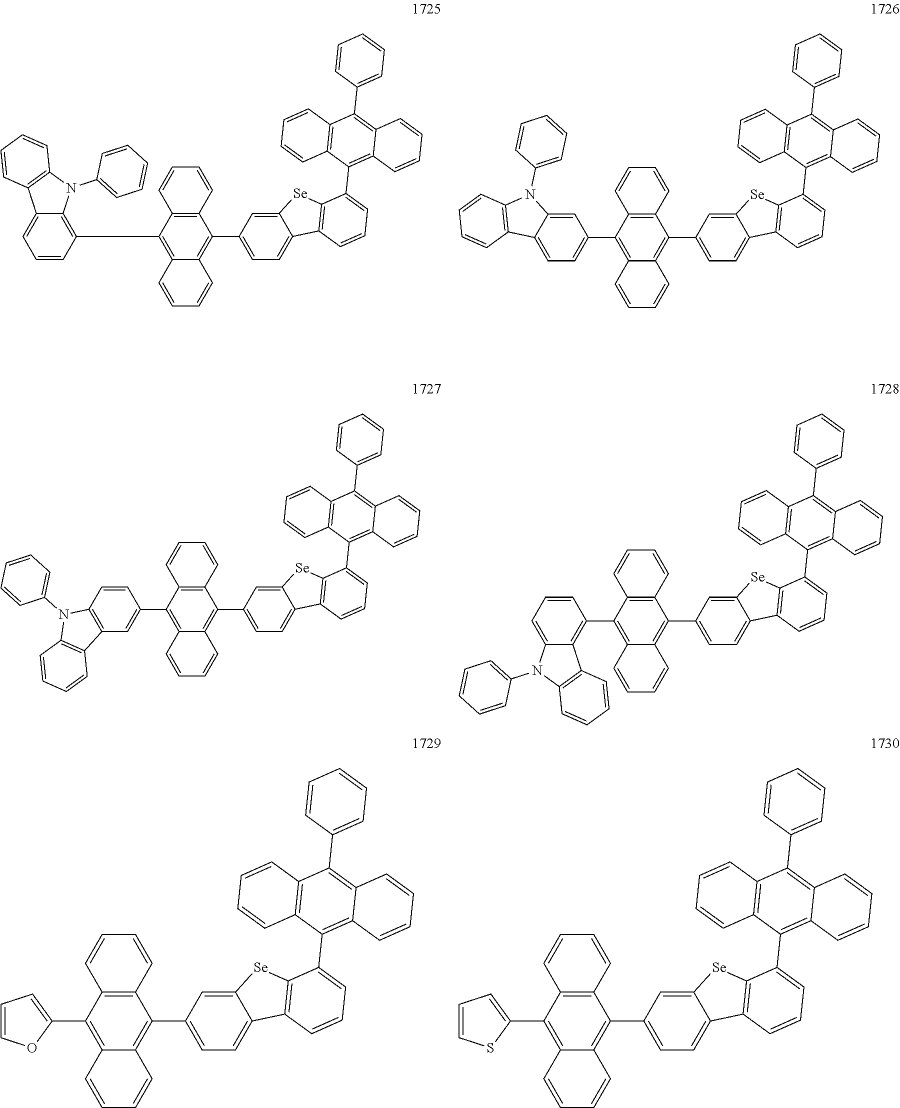

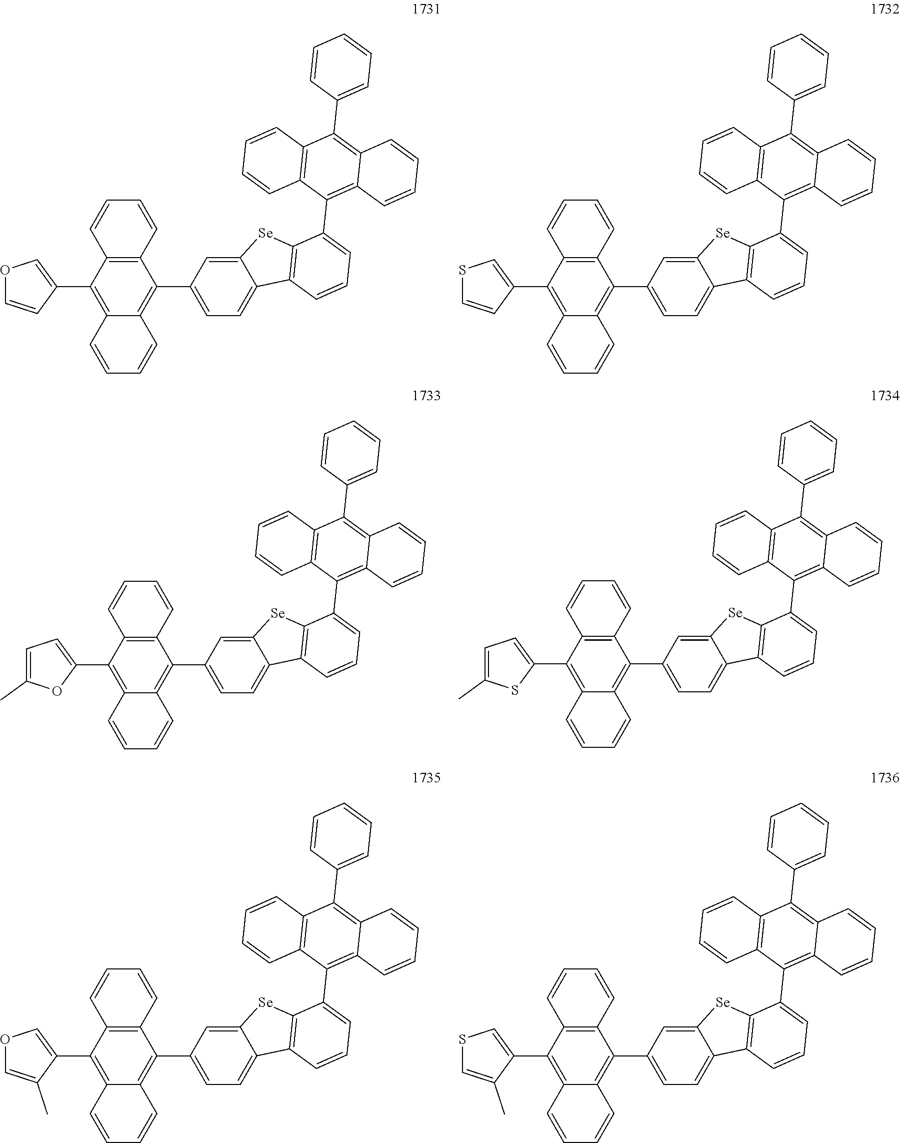

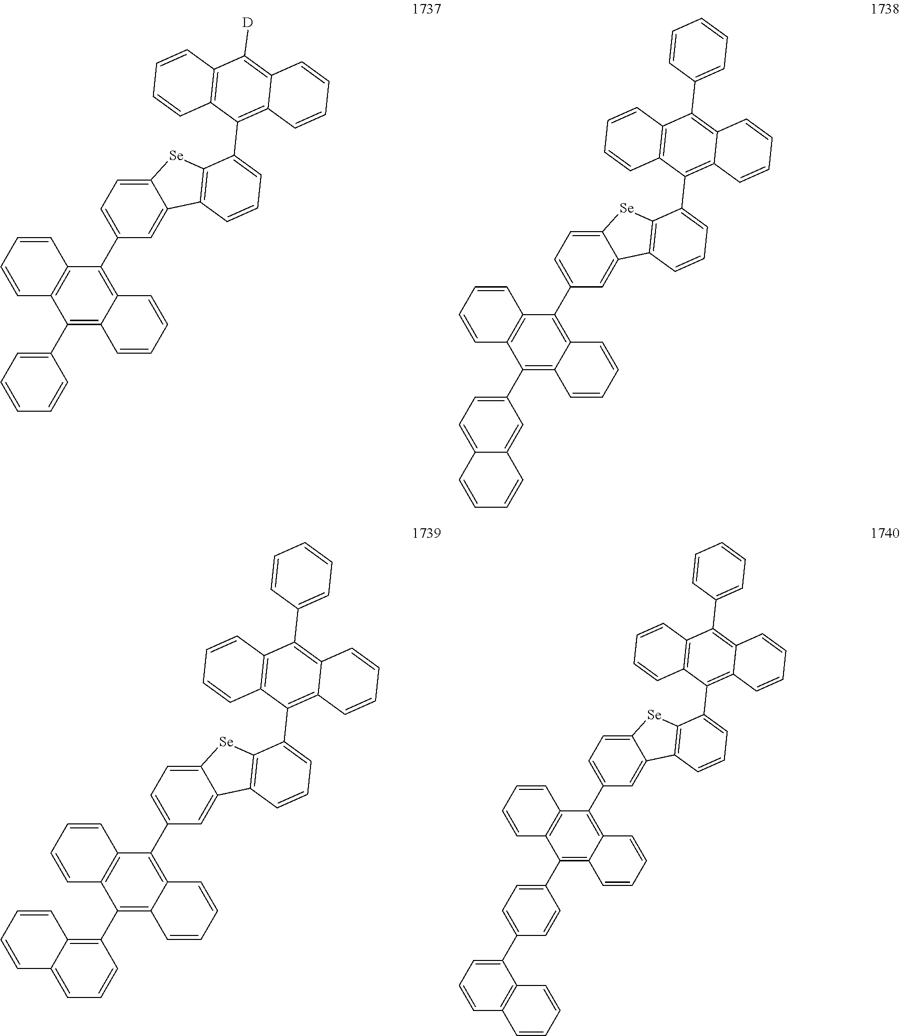 [SMILES: [2H]c1c2ccccc2c(-c2cccc3c2[se]c2ccc(-c4c5ccccc5c(-c5ccccc5)c5ccccc45)cc23)c2ccccc12 ; c1ccc(-c2c3ccccc3c(-c3cccc4c3[se]c3ccc(-c5c6ccccc6c(-c6ccc7ccccc7c6)c6ccccc56)cc34)c3ccccc23)cc1 ; c1ccc(-c2c3ccccc3c(-c3cccc4c3[se]c3ccc(-c5c6ccccc6c(-c6cccc7ccccc67)c6ccccc56)cc34)c3ccccc23)cc1 ; c1ccc(-c2c3ccccc3c(-c3cccc4c3[se]c3ccc(-c5c6ccccc6c(-c6ccc(-c7cccc8ccccc78)cc6)c6ccccc56)cc34)c3ccccc23)cc1]

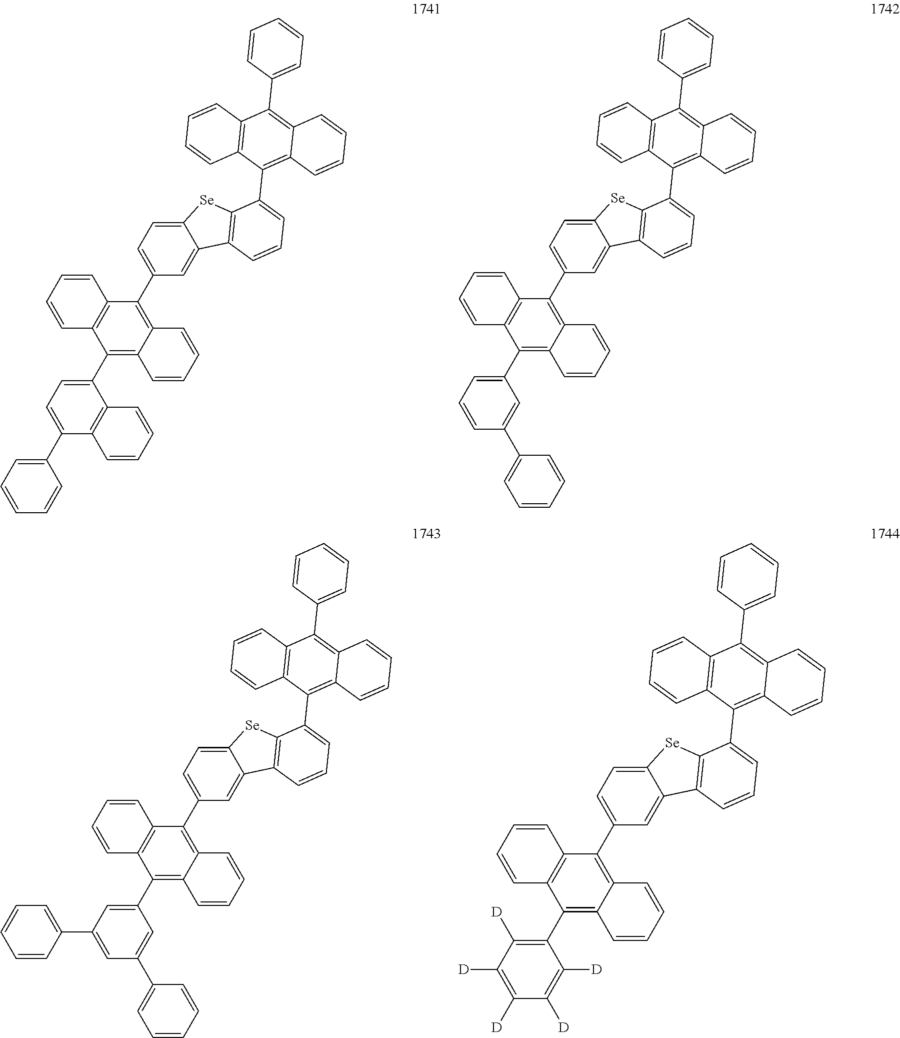 [SMILES: c1ccc(-c2ccc(-c3c4ccccc4c(-c4ccc5[se]c6c(-c7c8ccccc8c(-c8ccccc8)c8ccccc78)cccc6c5c4)c4ccccc34)c3ccccc23)cc1 ; c1ccc(-c2cccc(-c3c4ccccc4c(-c4ccc5[se]c6c(-c7c8ccccc8c(-c8ccccc8)c8ccccc78)cccc6c5c4)c4ccccc34)c2)cc1 ; c1ccc(-c2cc(-c3ccccc3)cc(-c3c4ccccc4c(-c4ccc5[se]c6c(-c7c8ccccc8c(-c8ccccc8)c8ccccc78)cccc6c5c4)c4ccccc34)c2)cc1 ; [2H]c1c([2H])c([2H])c(-c2c3ccccc3c(-c3ccc4[se]c5c(-c6c7ccccc7c(-c7ccccc7)c7ccccc67)cccc5c4c3)c3ccccc23)c([2H])c1[2H]]

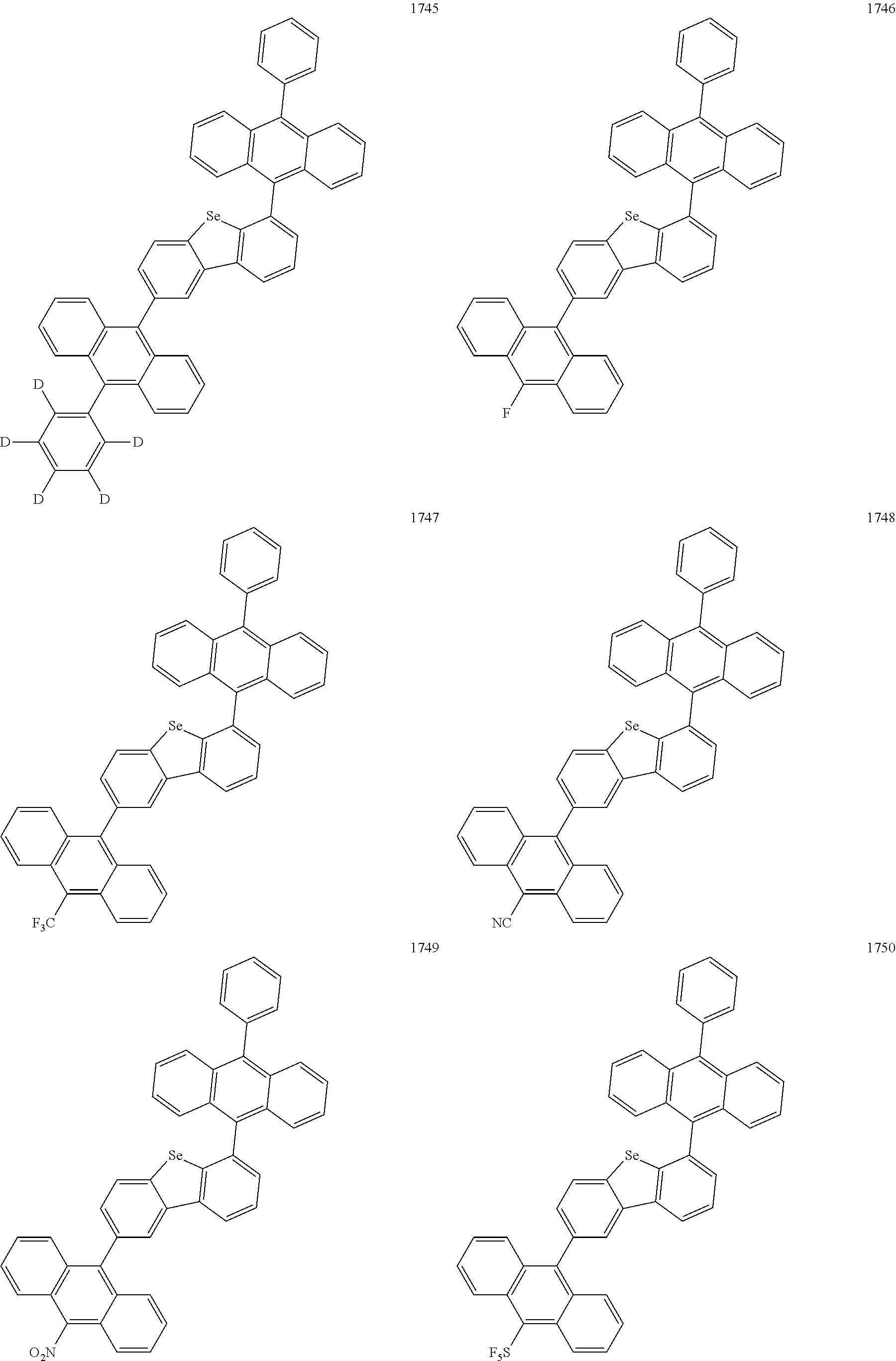

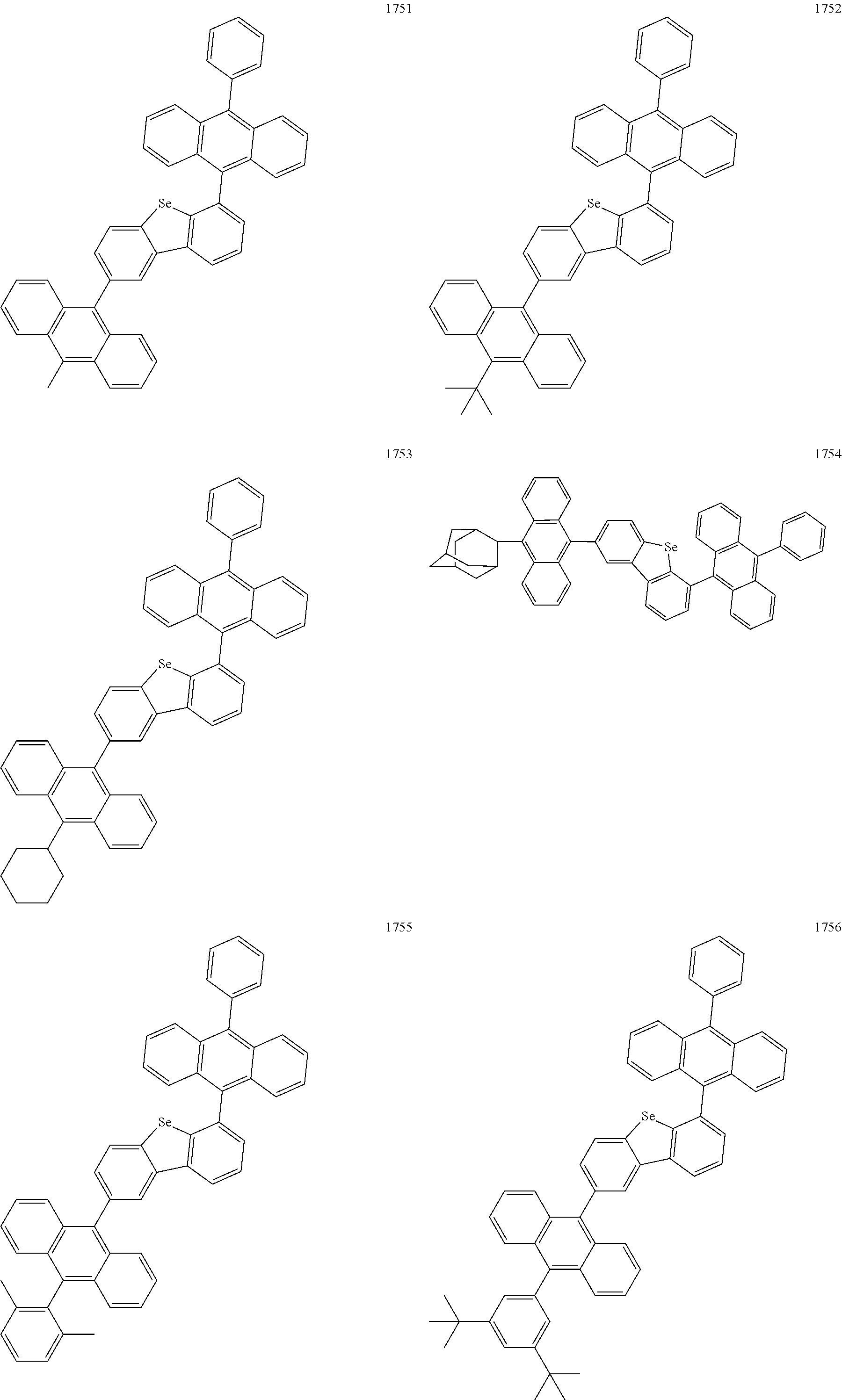

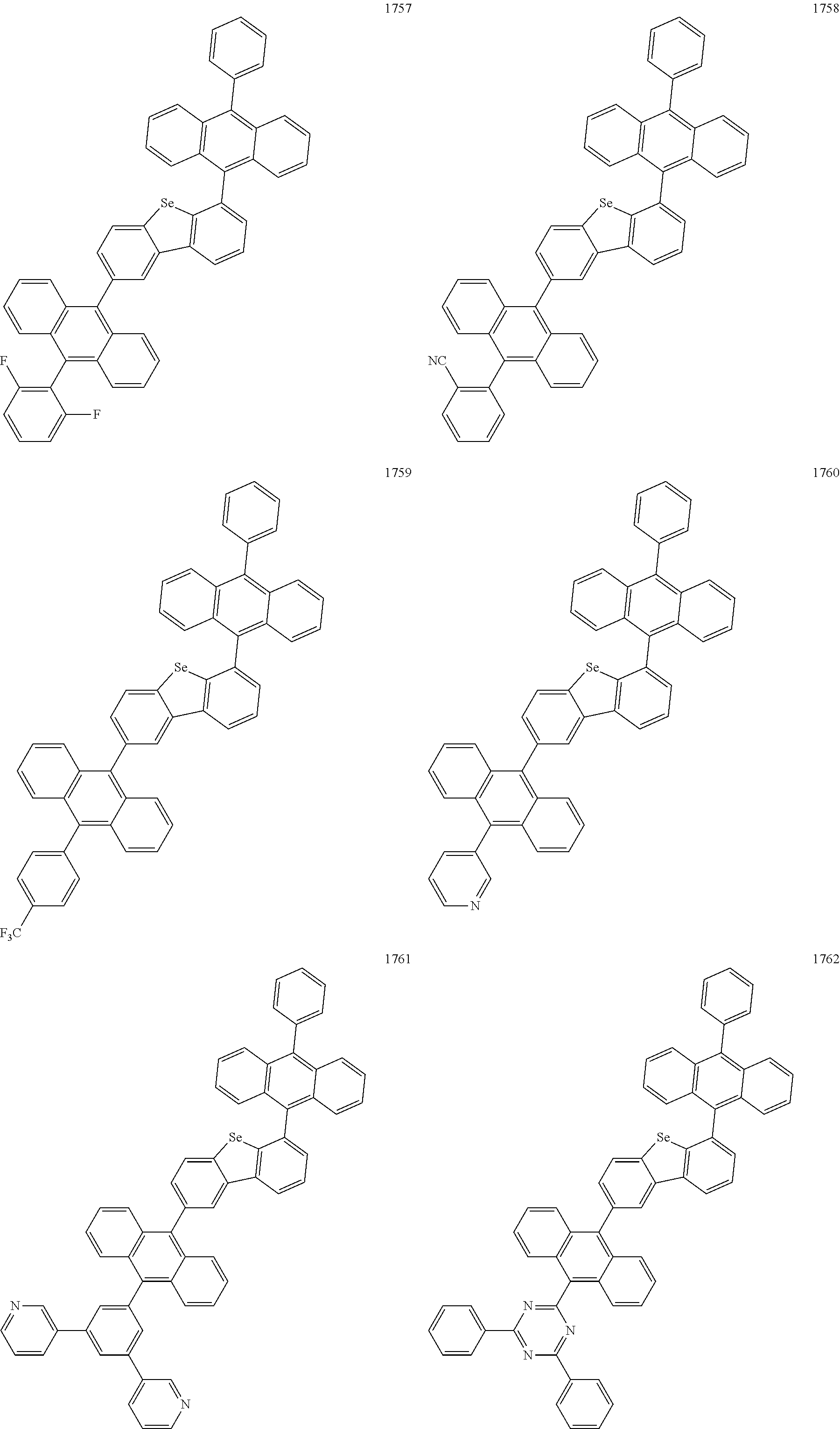

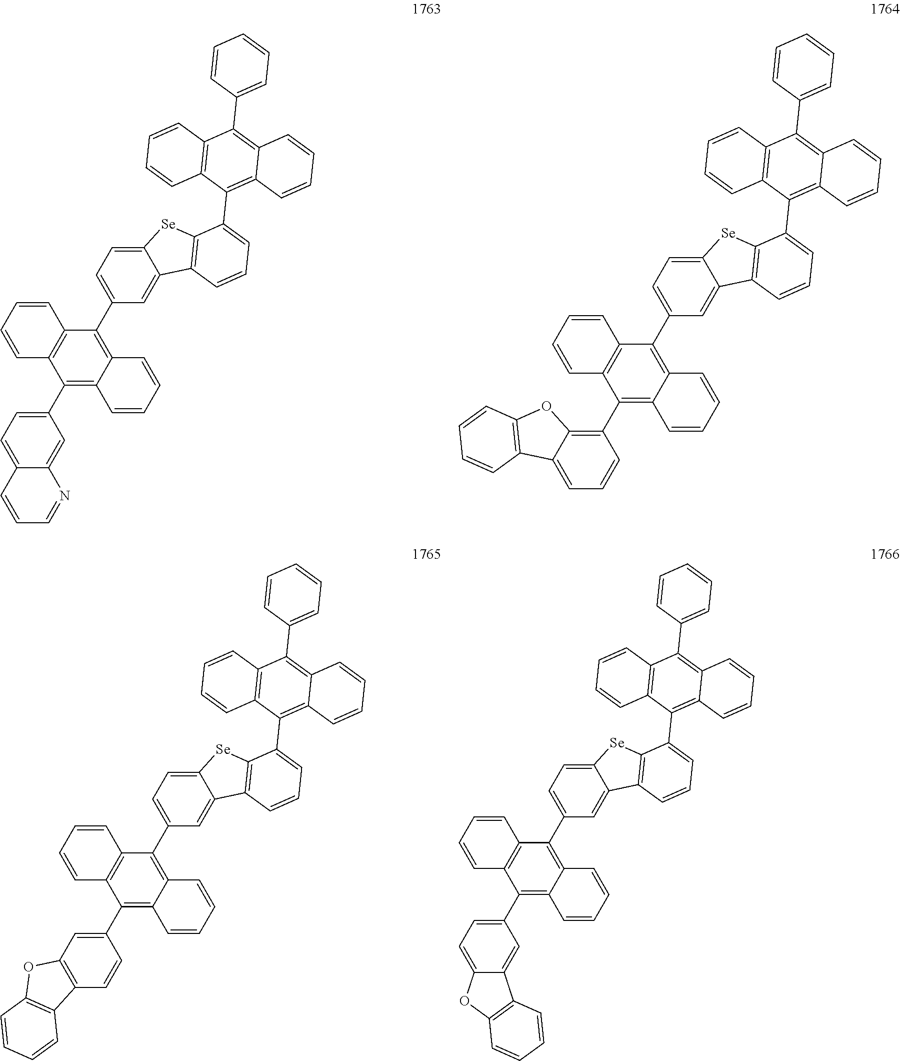 [SMILES: c1ccc(-c2c3ccccc3c(-c3cccc4c3[se]c3ccc(-c5c6ccccc6c(-c6ccc7cccnc7c6)c6ccccc56)cc34)c3ccccc23)cc1 ; c1ccc(-c2c3ccccc3c(-c3cccc4c3[se]c3ccc(-c5c6ccccc6c(-c6cccc7c6oc6ccccc67)c6ccccc56)cc34)c3ccccc23)cc1 ; c1ccc(-c2c3ccccc3c(-c3cccc4c3[se]c3ccc(-c5c6ccccc6c(-c6ccc7c(c6)oc6ccccc67)c6ccccc56)cc34)c3ccccc23)cc1 ; c1ccc(-c2c3ccccc3c(-c3cccc4c3[se]c3ccc(-c5c6ccccc6c(-c6ccc7oc8ccccc8c7c6)c6ccccc56)cc34)c3ccccc23)cc1]

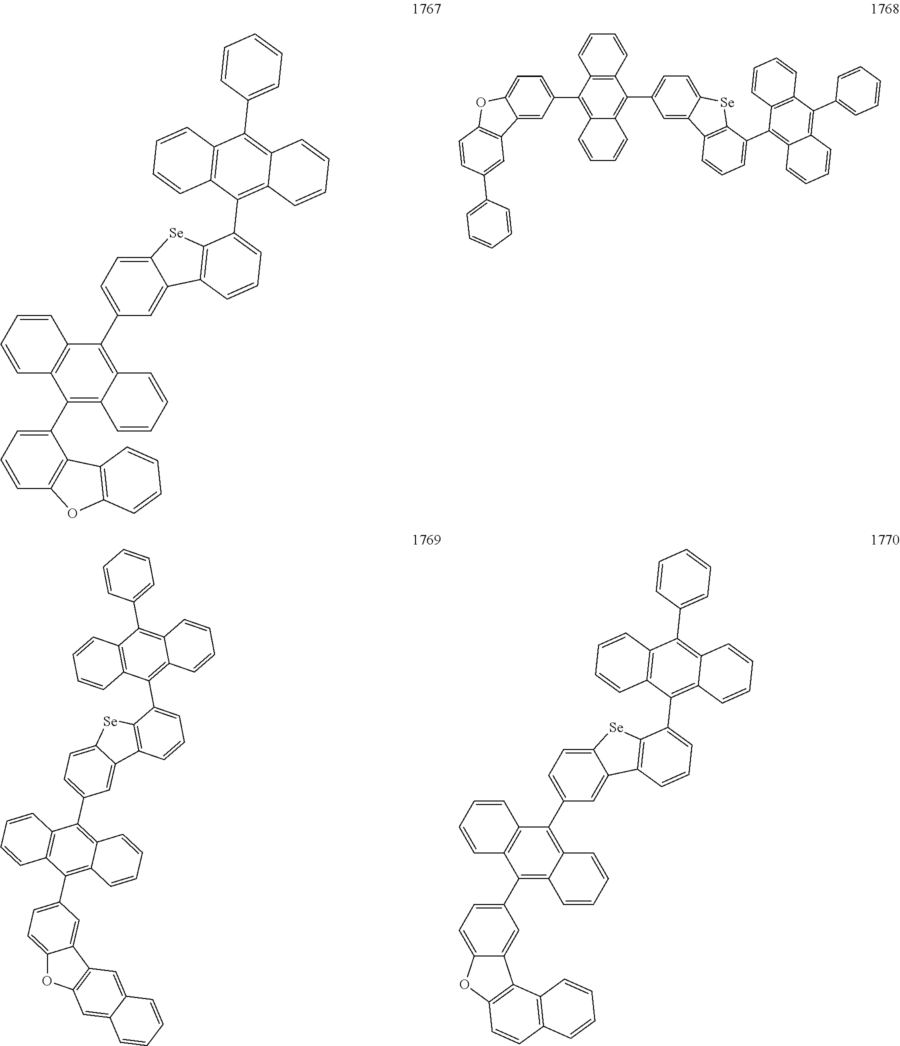 [SMILES: c1ccc(-c2c3ccccc3c(-c3cccc4c3[se]c3ccc(-c5c6ccccc6c(-c6cccc7oc8ccccc8c67)c6ccccc56)cc34)c3ccccc23)cc1 ; c1ccc(-c2ccc3oc4ccc(-c5c6ccccc6c(-c6ccc7[se]c8c(-c9c%10ccccc%10c(-c%10ccccc%10)c%10ccccc9%10)cccc8c7c6)c6ccccc56)cc4c3c2)cc1 ; c1ccc(-c2c3ccccc3c(-c3cccc4c3[se]c3ccc(-c5c6ccccc6c(-c6ccc7oc8cc9ccccc9cc8c7c6)c6ccccc56)cc34)c3ccccc23)cc1 ; c1ccc(-c2c3ccccc3c(-c3cccc4c3[se]c3ccc(-c5c6ccccc6c(-c6ccc7oc8ccc9ccccc9c8c7c6)c6ccccc56)cc34)c3ccccc23)cc1]

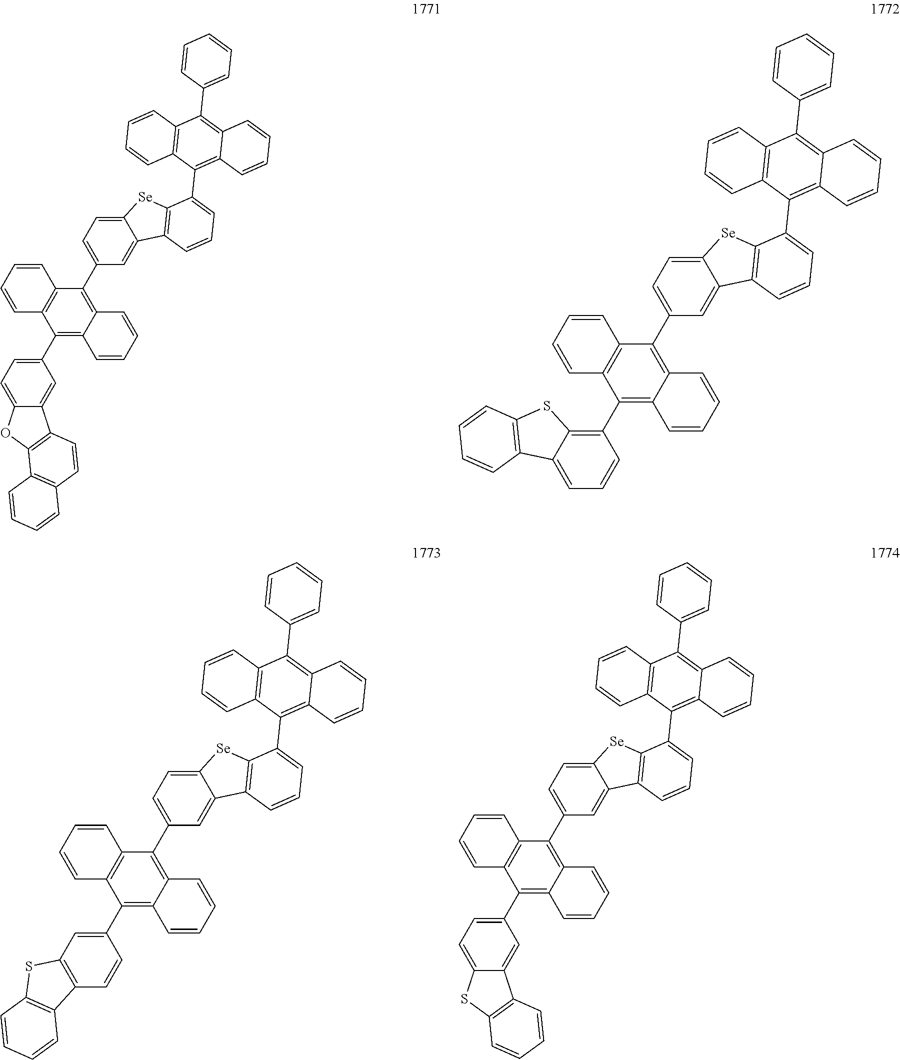 [SMILES: c1ccc(-c2c3ccccc3c(-c3cccc4c3[se]c3ccc(-c5c6ccccc6c(-c6ccc7oc8c9ccccc9ccc8c7c6)c6ccccc56)cc34)c3ccccc23)cc1 ; c1ccc(-c2c3ccccc3c(-c3cccc4c3[se]c3ccc(-c5c6ccccc6c(-c6cccc7c6sc6ccccc67)c6ccccc56)cc34)c3ccccc23)cc1 ; c1ccc(-c2c3ccccc3c(-c3cccc4c3[se]c3ccc(-c5c6ccccc6c(-c6ccc7c(c6)sc6ccccc67)c6ccccc56)cc34)c3ccccc23)cc1 ; c1ccc(-c2c3ccccc3c(-c3cccc4c3[se]c3ccc(-c5c6ccccc6c(-c6ccc7sc8ccccc8c7c6)c6ccccc56)cc34)c3ccccc23)cc1]

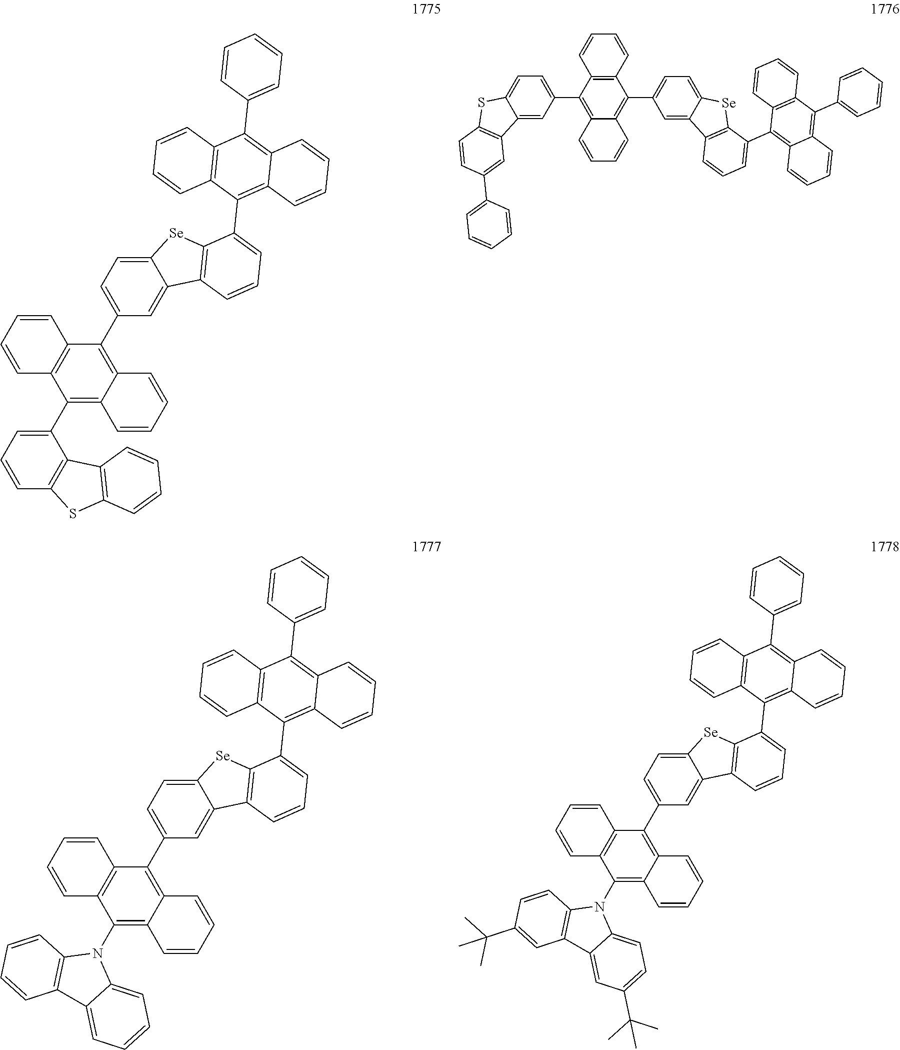 [SMILES: c1ccc(-c2c3ccccc3c(-c3cccc4c3[se]c3ccc(-c5c6ccccc6c(-c6cccc7sc8ccccc8c67)c6ccccc56)cc34)c3ccccc23)cc1 ; c1ccc(-c2ccc3sc4ccc(-c5c6ccccc6c(-c6ccc7[se]c8c(-c9c%10ccccc%10c(-c%10ccccc%10)c%10ccccc9%10)cccc8c7c6)c6ccccc56)cc4c3c2)cc1 ; c1ccc(-c2c3ccccc3c(-c3cccc4c3[se]c3ccc(-c5c6ccccc6c(-n6c7ccccc7c7ccccc76)c6ccccc56)cc34)c3ccccc23)cc1 ; CC(C)(C)c1ccc2c(c1)c1cc(C(C)(C)C)ccc1n2-c1c2ccccc2c(-c2ccc3[se]c4c(-c5c6ccccc6c(-c6ccccc6)c6ccccc56)cccc4c3c2)c2ccccc12]

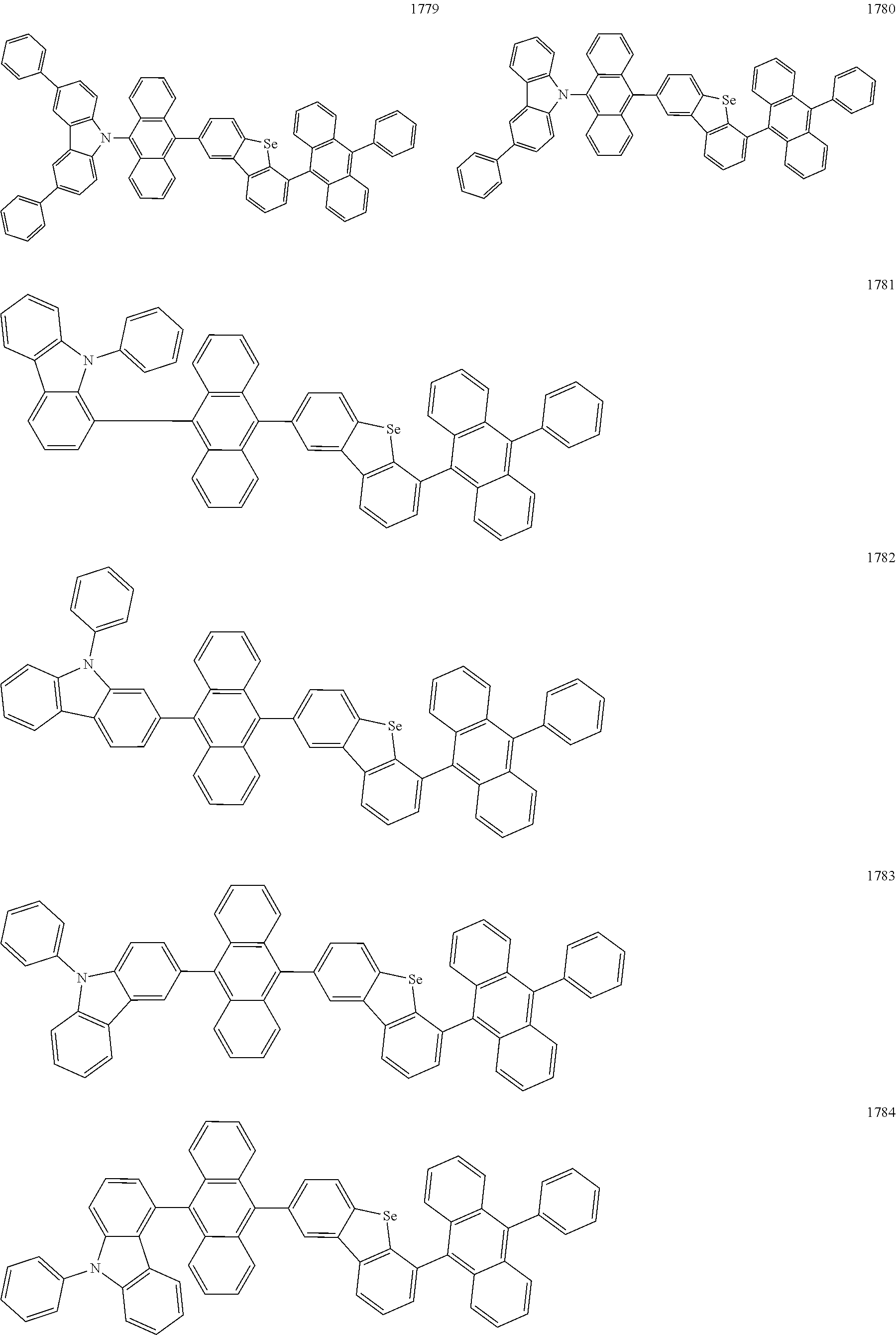

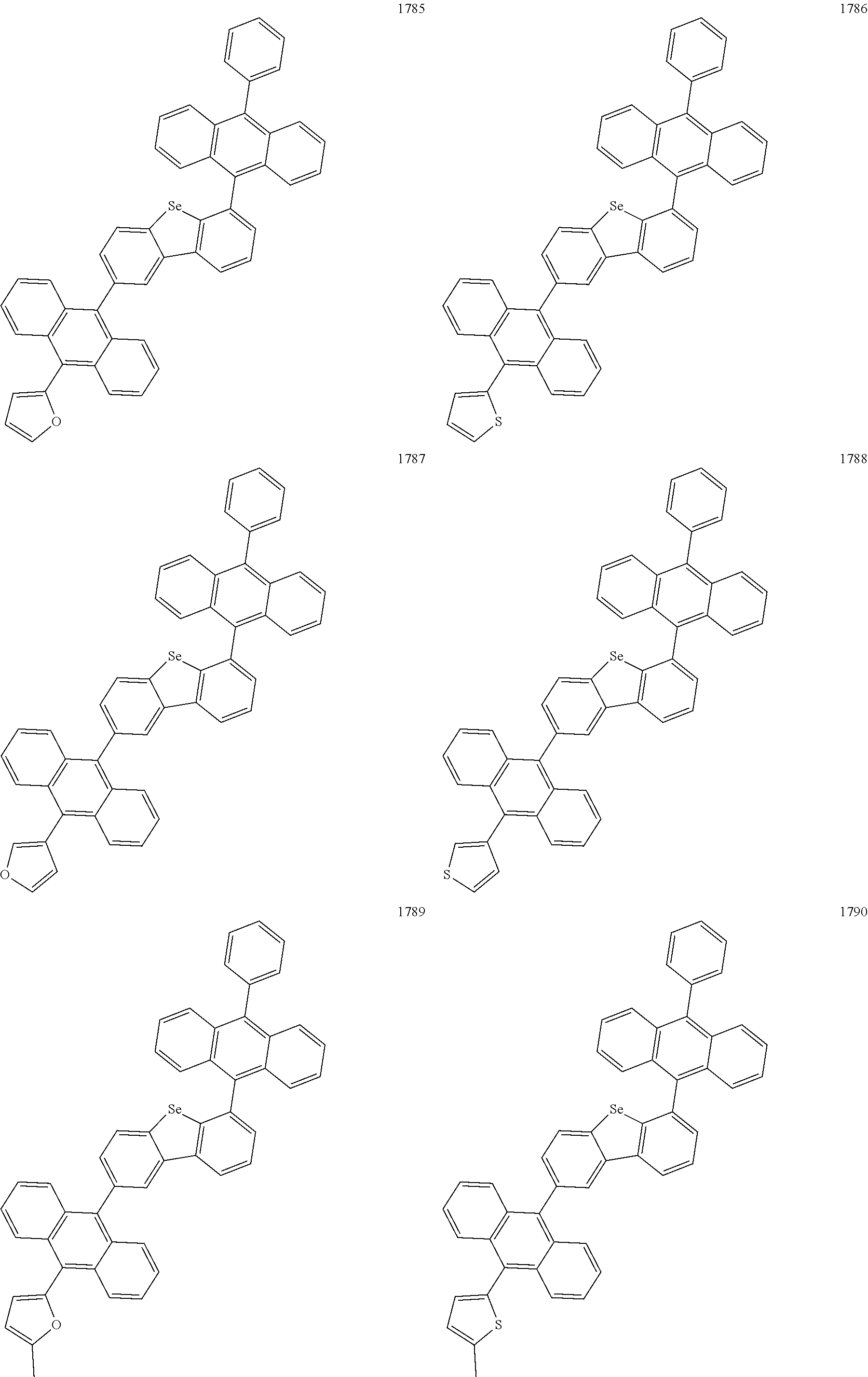

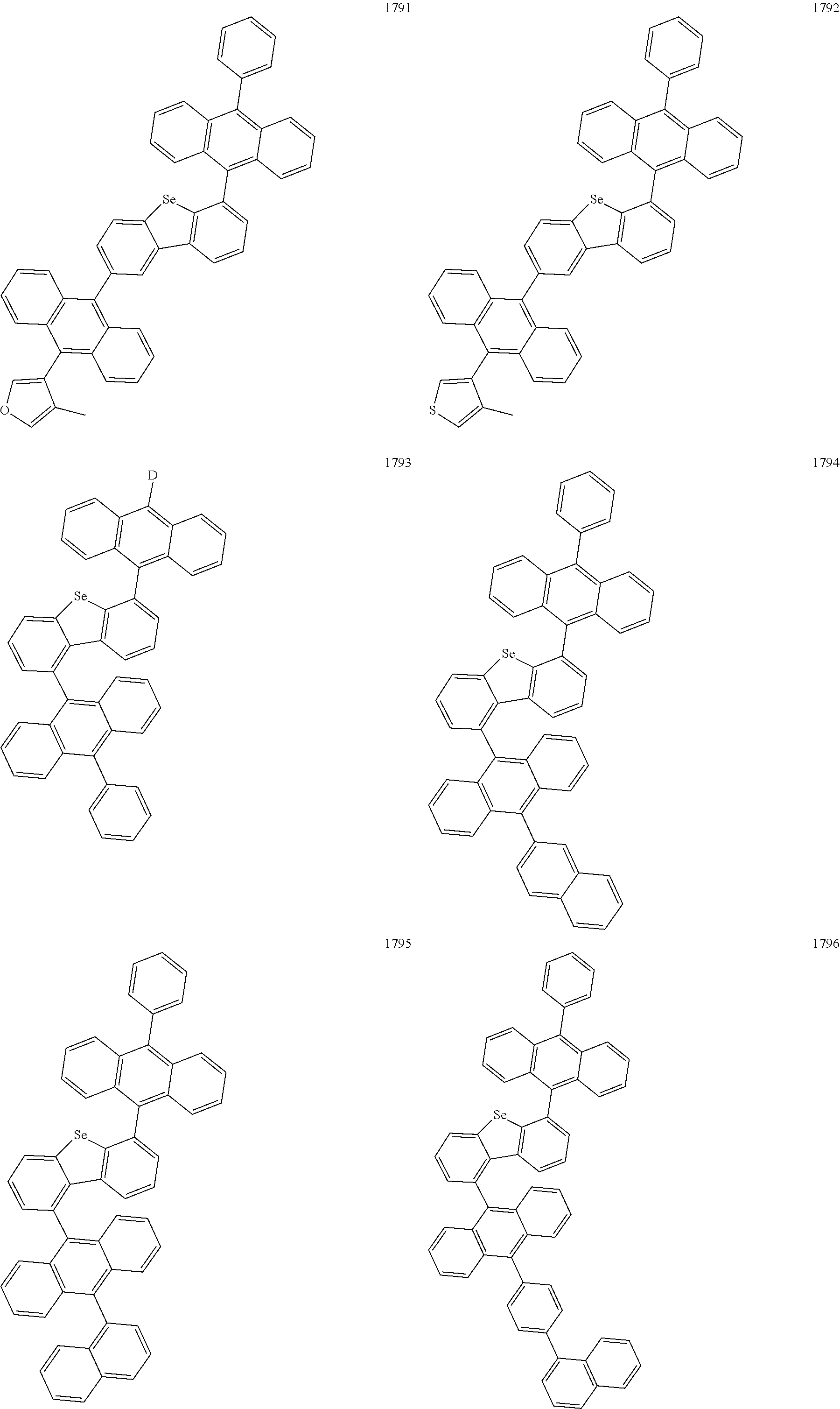

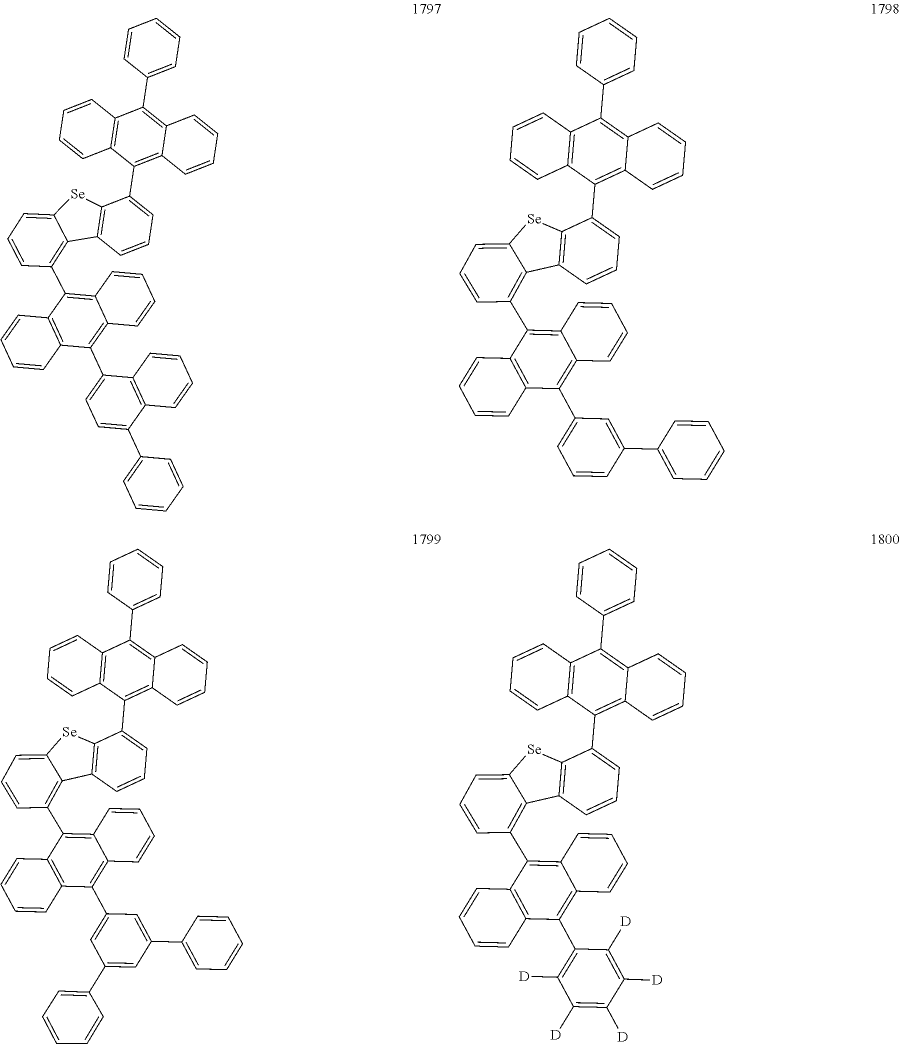

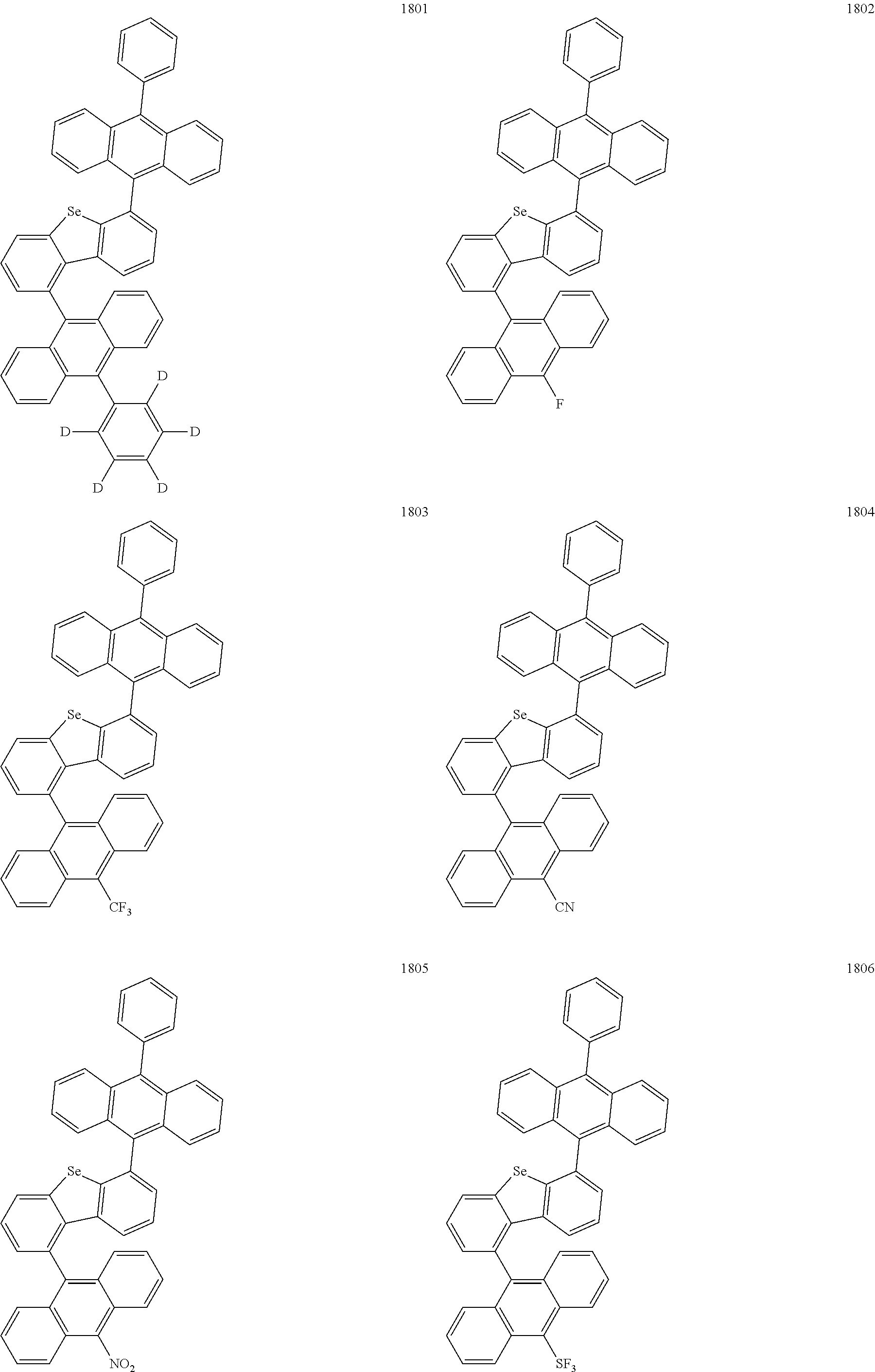

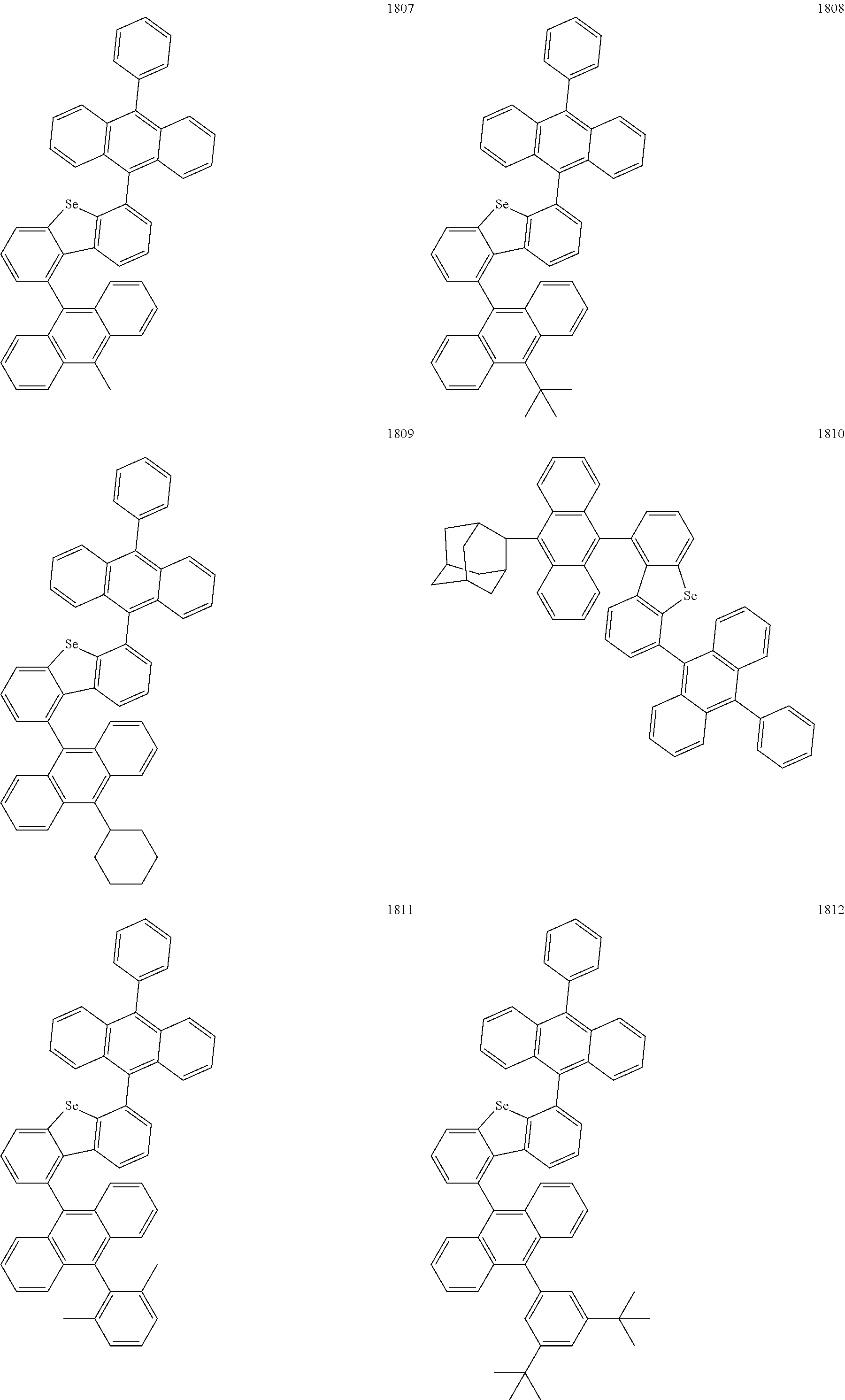

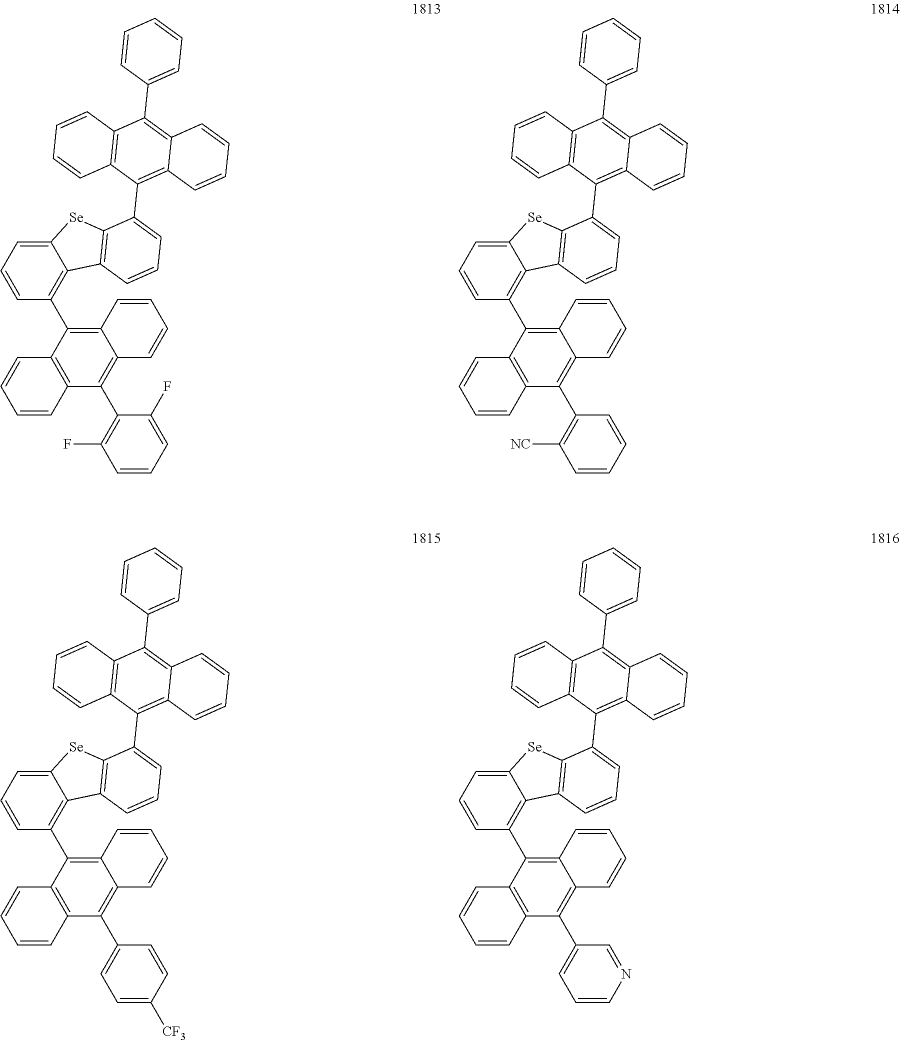 [SMILES: Fc1cccc(F)c1-c1c2ccccc2c(-c2cccc3[se]c4c(-c5c6ccccc6c(-c6ccccc6)c6ccccc56)cccc4c23)c2ccccc12 ; N#Cc1ccccc1-c1c2ccccc2c(-c2cccc3[se]c4c(-c5c6ccccc6c(-c6ccccc6)c6ccccc56)cccc4c23)c2ccccc12 ; FC(F)(F)c1ccc(-c2c3ccccc3c(-c3cccc4[se]c5c(-c6c7ccccc7c(-c7ccccc7)c7ccccc67)cccc5c34)c3ccccc23)cc1 ; c1ccc(-c2c3ccccc3c(-c3cccc4c3[se]c3cccc(-c5c6ccccc6c(-c6cccnc6)c6ccccc56)c34)c3ccccc23)cc1]

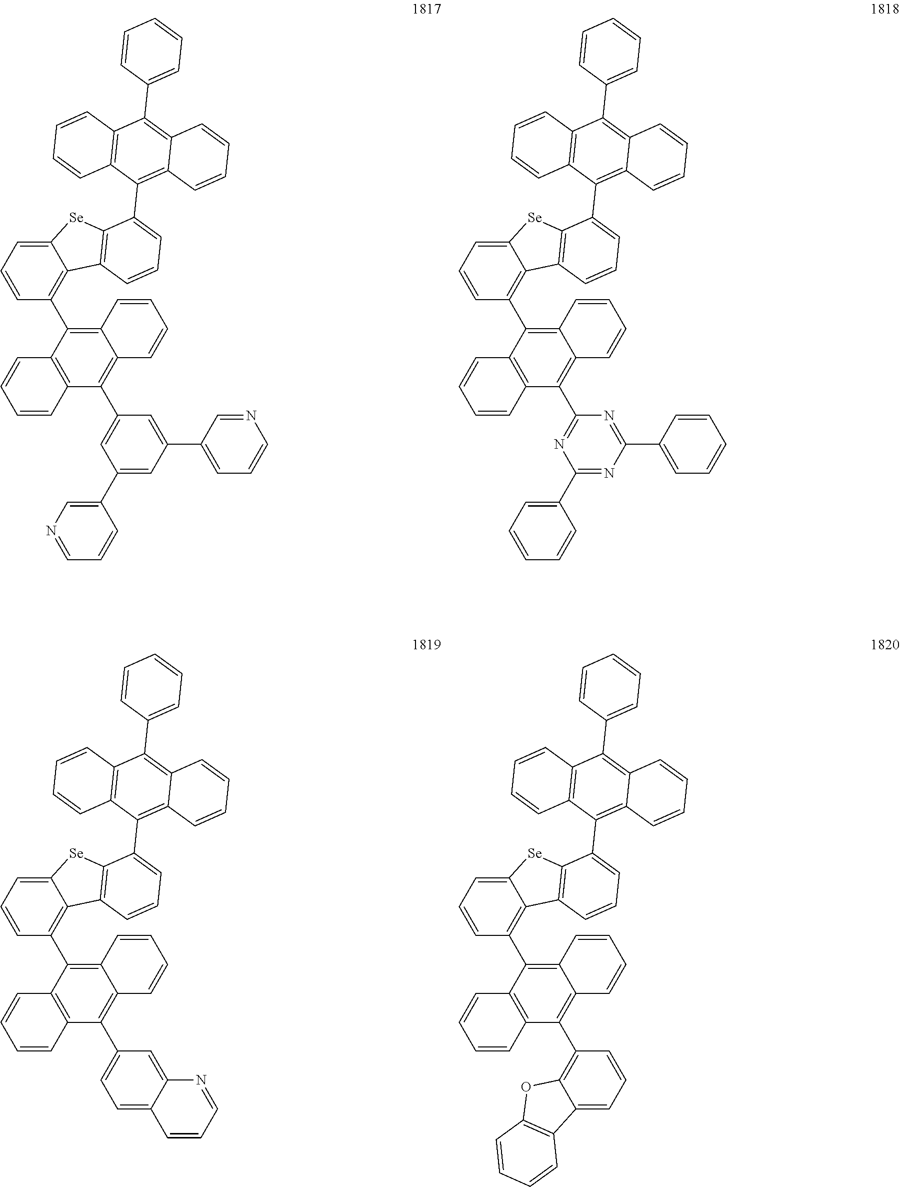 [SMILES: c1ccc(-c2c3ccccc3c(-c3cccc4c3[se]c3cccc(-c5c6ccccc6c(-c6cc(-c7cccnc7)cc(-c7cccnc7)c6)c6ccccc56)c34)c3ccccc23)cc1 ; c1ccc(-c2nc(-c3ccccc3)nc(-c3c4ccccc4c(-c4cccc5[se]c6c(-c7c8ccccc8c(-c8ccccc8)c8ccccc78)cccc6c45)c4ccccc34)n2)cc1 ; c1ccc(-c2c3ccccc3c(-c3cccc4c3[se]c3cccc(-c5c6ccccc6c(-c6ccc7cccnc7c6)c6ccccc56)c34)c3ccccc23)cc1 ; c1ccc(-c2c3ccccc3c(-c3cccc4c3[se]c3cccc(-c5c6ccccc6c(-c6cccc7c6oc6ccccc67)c6ccccc56)c34)c3ccccc23)cc1]

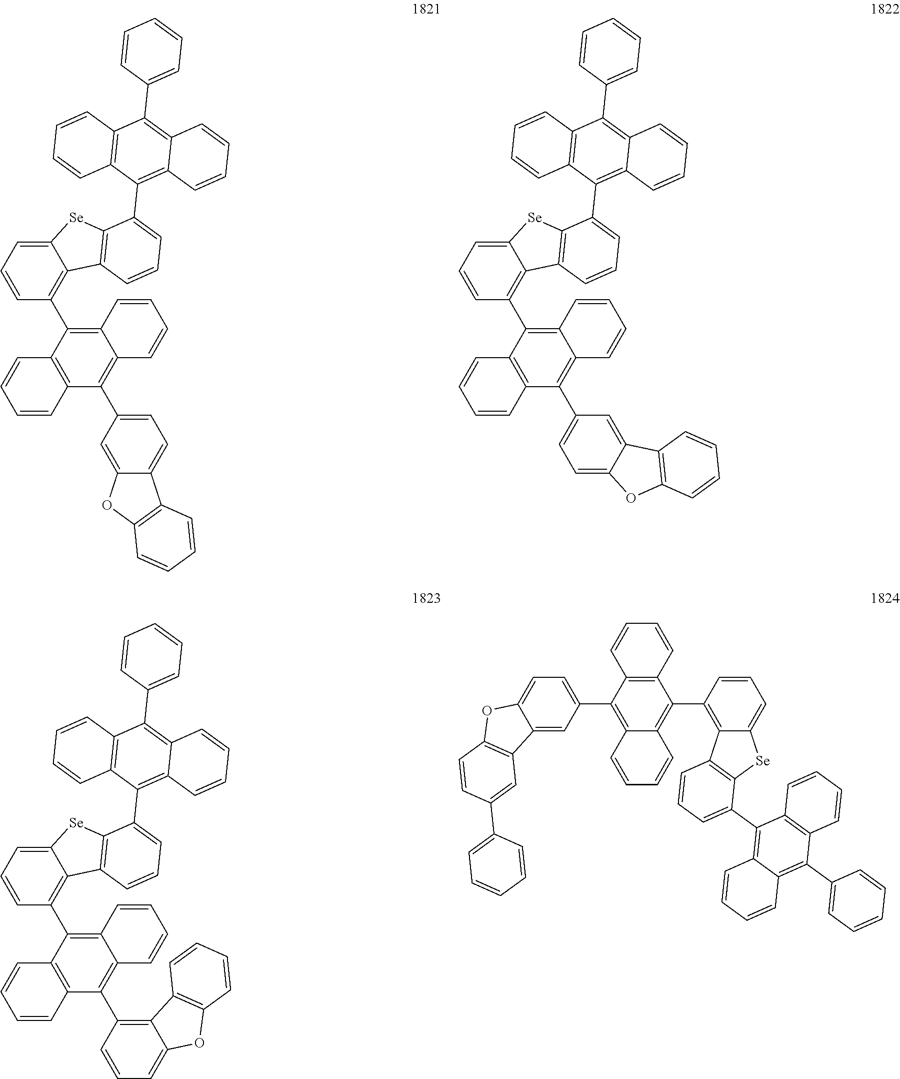 [SMILES: c1ccc(-c2c3ccccc3c(-c3cccc4c3[se]c3cccc(-c5c6ccccc6c(-c6ccc7c(c6)oc6ccccc67)c6ccccc56)c34)c3ccccc23)cc1 ; c1ccc(-c2c3ccccc3c(-c3cccc4c3[se]c3cccc(-c5c6ccccc6c(-c6ccc7oc8ccccc8c7c6)c6ccccc56)c34)c3ccccc23)cc1 ; c1ccc(-c2c3ccccc3c(-c3cccc4c3[se]c3cccc(-c5c6ccccc6c(-c6cccc7oc8ccccc8c67)c6ccccc56)c34)c3ccccc23)cc1 ; c1ccc(-c2ccc3oc4ccc(-c5c6ccccc6c(-c6cccc7[se]c8c(-c9c%10ccccc%10c(-c%10ccccc%10)c%10ccccc9%10)cccc8c67)c6ccccc56)cc4c3c2)cc1]

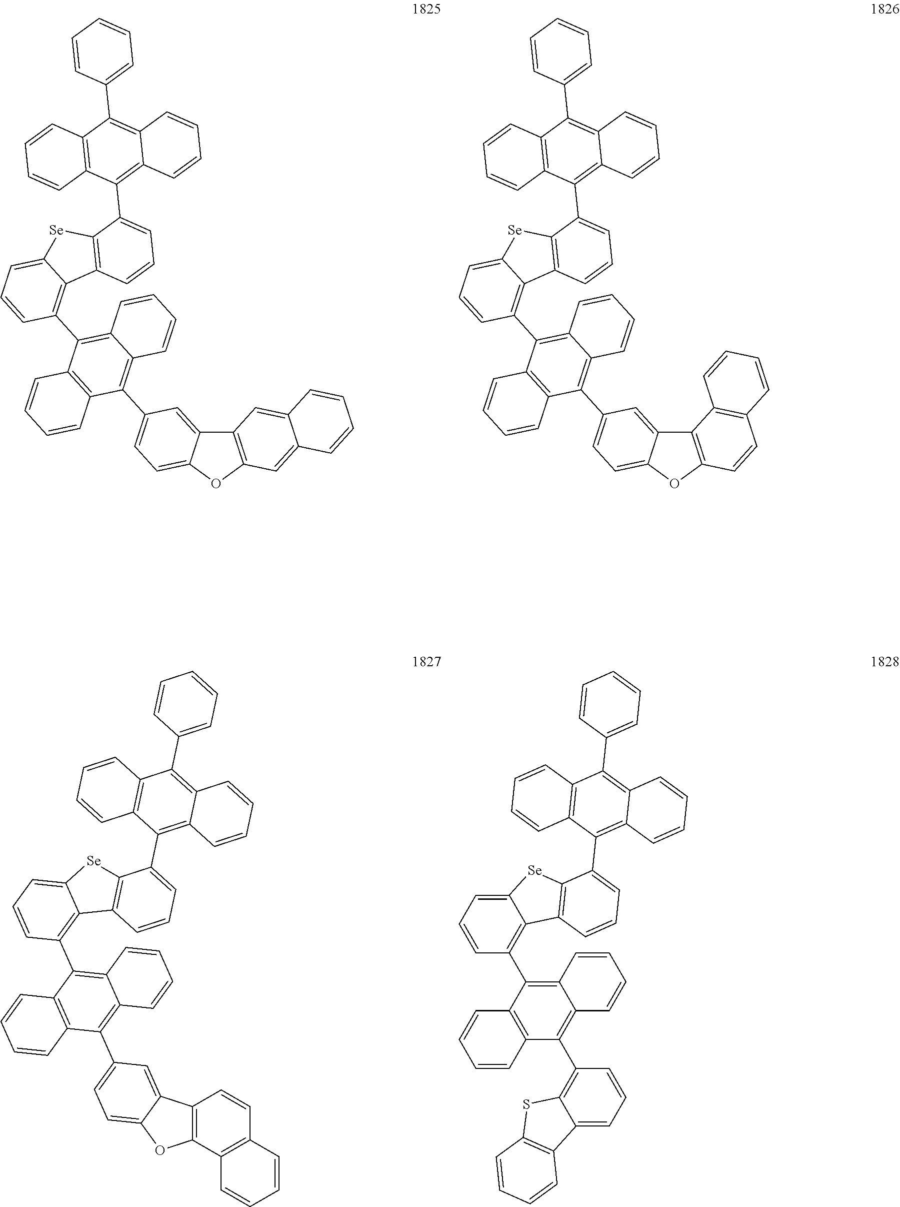 [SMILES: c1ccc(-c2c3ccccc3c(-c3cccc4c3[se]c3cccc(-c5c6ccccc6c(-c6ccc7oc8cc9ccccc9cc8c7c6)c6ccccc56)c34)c3ccccc23)cc1 ; c1ccc(-c2c3ccccc3c(-c3cccc4c3[se]c3cccc(-c5c6ccccc6c(-c6ccc7oc8ccc9ccccc9c8c7c6)c6ccccc56)c34)c3ccccc23)cc1 ; c1ccc(-c2c3ccccc3c(-c3cccc4c3[se]c3cccc(-c5c6ccccc6c(-c6ccc7oc8c9ccccc9ccc8c7c6)c6ccccc56)c34)c3ccccc23)cc1 ; c1ccc(-c2c3ccccc3c(-c3cccc4c3[se]c3cccc(-c5c6ccccc6c(-c6cccc7c6sc6ccccc67)c6ccccc56)c34)c3ccccc23)cc1]

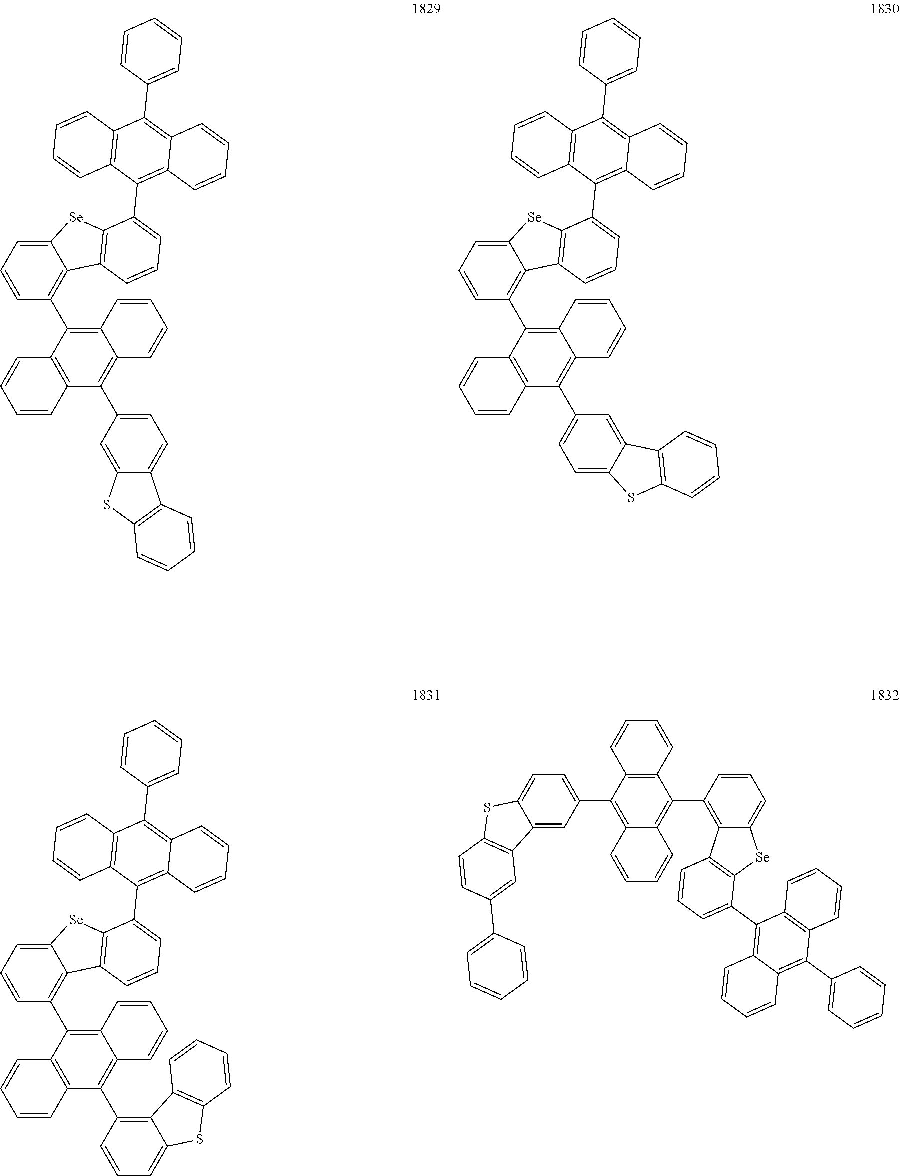 [SMILES: c1ccc(-c2c3ccccc3c(-c3cccc4c3[se]c3cccc(-c5c6ccccc6c(-c6ccc7c(c6)sc6ccccc67)c6ccccc56)c34)c3ccccc23)cc1 ; c1ccc(-c2c3ccccc3c(-c3cccc4c3[se]c3cccc(-c5c6ccccc6c(-c6ccc7sc8ccccc8c7c6)c6ccccc56)c34)c3ccccc23)cc1 ; c1ccc(-c2c3ccccc3c(-c3cccc4c3[se]c3cccc(-c5c6ccccc6c(-c6cccc7sc8ccccc8c67)c6ccccc56)c34)c3ccccc23)cc1 ; c1ccc(-c2ccc3sc4ccc(-c5c6ccccc6c(-c6cccc7[se]c8c(-c9c%10ccccc%10c(-c%10ccccc%10)c%10ccccc9%10)cccc8c67)c6ccccc56)cc4c3c2)cc1]

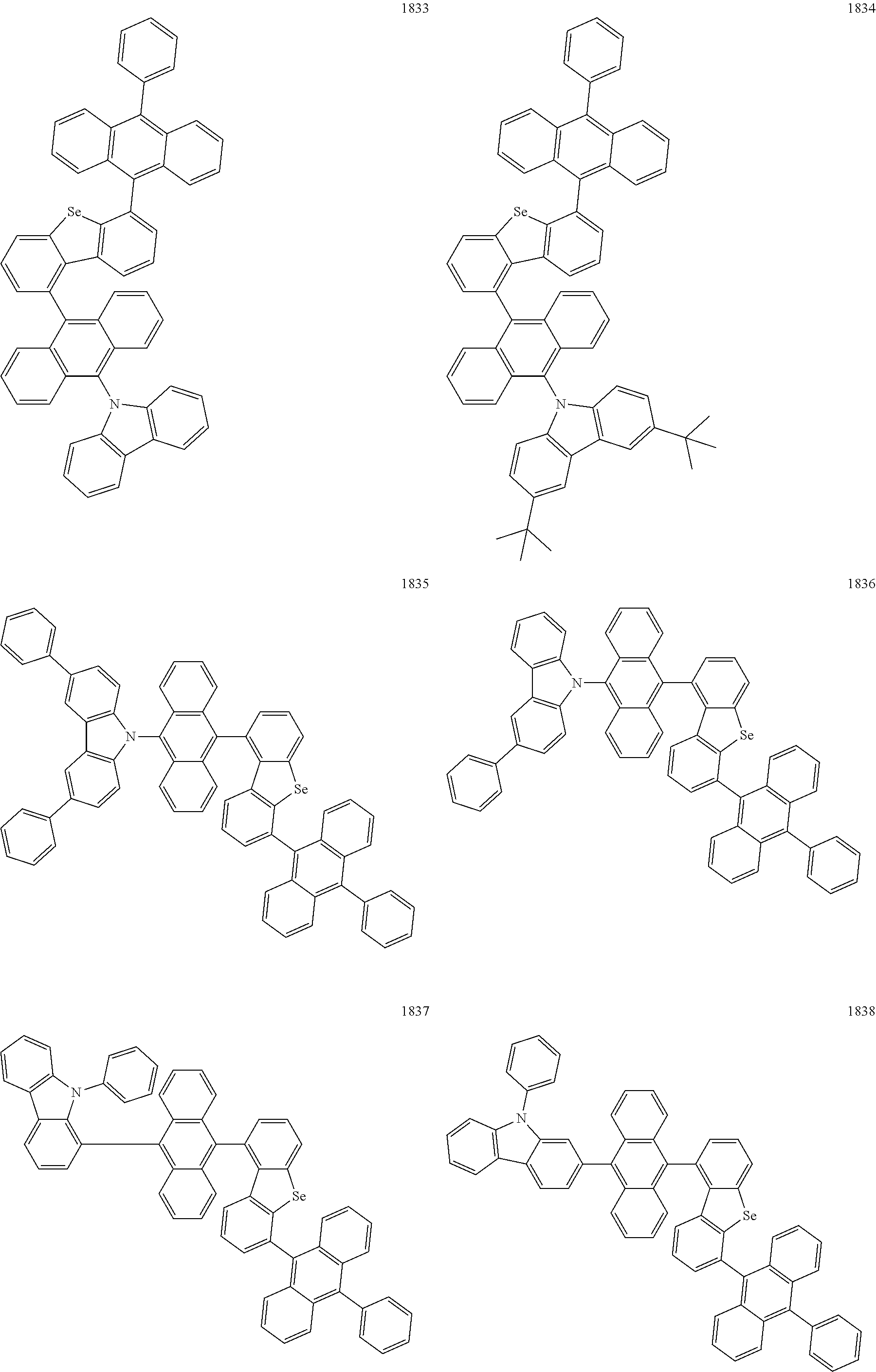

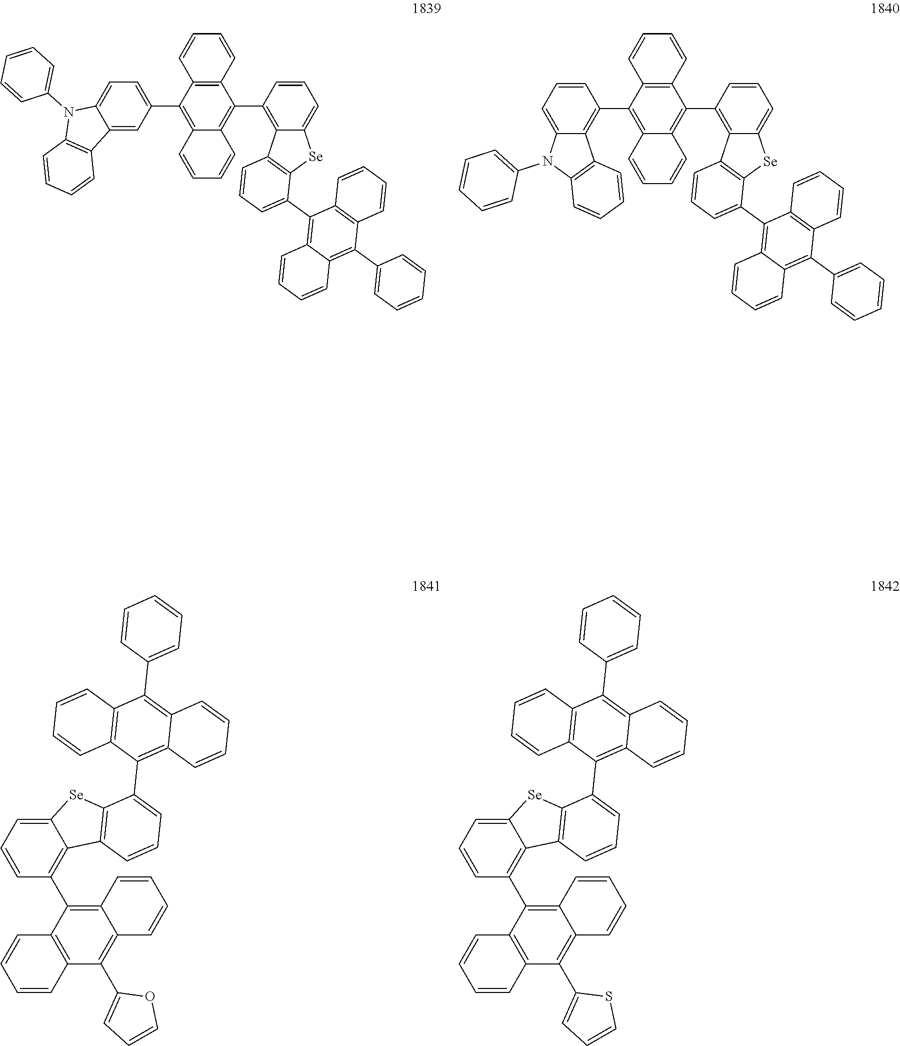 [SMILES: c1ccc(-c2c3ccccc3c(-c3cccc4c3[se]c3cccc(-c5c6ccccc6c(-c6ccc7c(c6)c6ccccc6n7-c6ccccc6)c6ccccc56)c34)c3ccccc23)cc1 ; c1ccc(-c2c3ccccc3c(-c3cccc4c3[se]c3cccc(-c5c6ccccc6c(-c6cccc7c6c6ccccc6n7-c6ccccc6)c6ccccc56)c34)c3ccccc23)cc1 ; c1ccc(-c2c3ccccc3c(-c3cccc4c3[se]c3cccc(-c5c6ccccc6c(-c6ccco6)c6ccccc56)c34)c3ccccc23)cc1 ; c1ccc(-c2c3ccccc3c(-c3cccc4c3[se]c3cccc(-c5c6ccccc6c(-c6cccs6)c6ccccc56)c34)c3ccccc23)cc1]

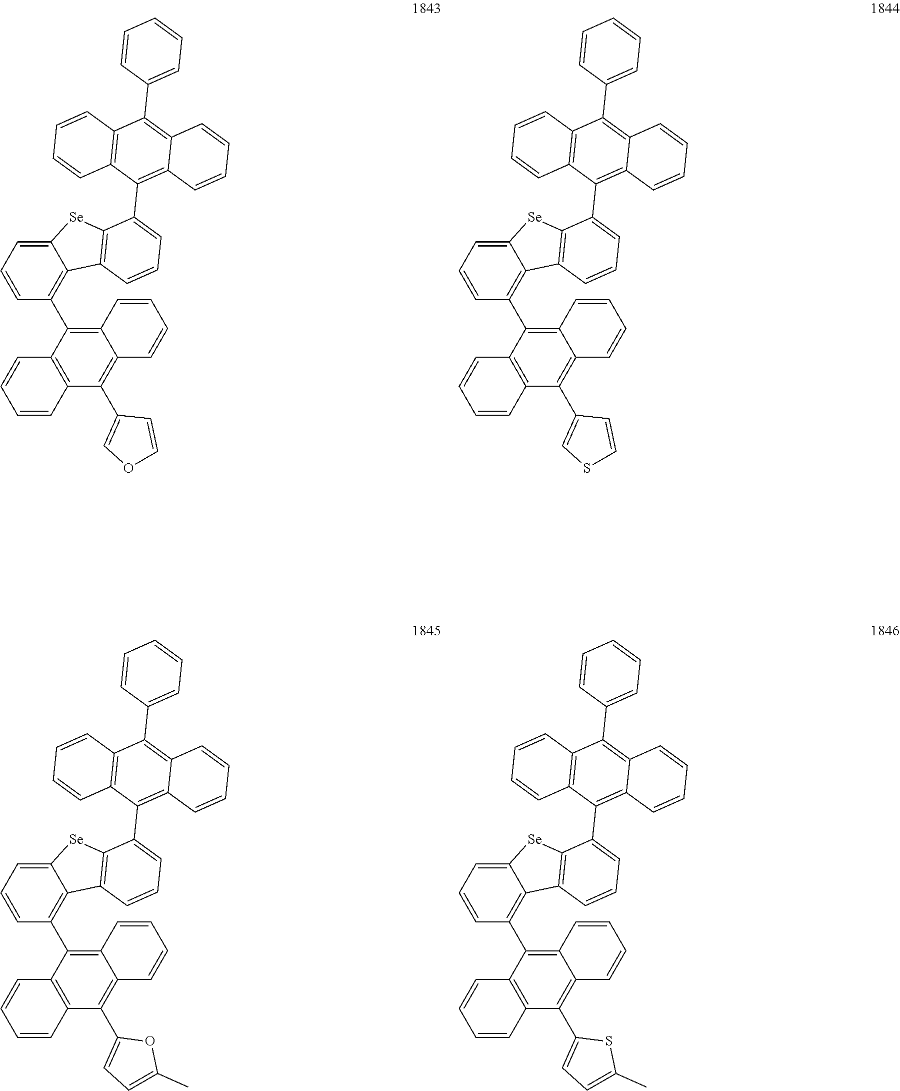 [SMILES: c1ccc(-c2c3ccccc3c(-c3cccc4c3[se]c3cccc(-c5c6ccccc6c(-c6ccoc6)c6ccccc56)c34)c3ccccc23)cc1 ; c1ccc(-c2c3ccccc3c(-c3cccc4c3[se]c3cccc(-c5c6ccccc6c(-c6ccsc6)c6ccccc56)c34)c3ccccc23)cc1 ; Cc1ccc(-c2c3ccccc3c(-c3cccc4[se]c5c(-c6c7ccccc7c(-c7ccccc7)c7ccccc67)cccc5c34)c3ccccc23)o1 ; Cc1ccc(-c2c3ccccc3c(-c3cccc4[se]c5c(-c6c7ccccc7c(-c7ccccc7)c7ccccc67)cccc5c34)c3ccccc23)s1]

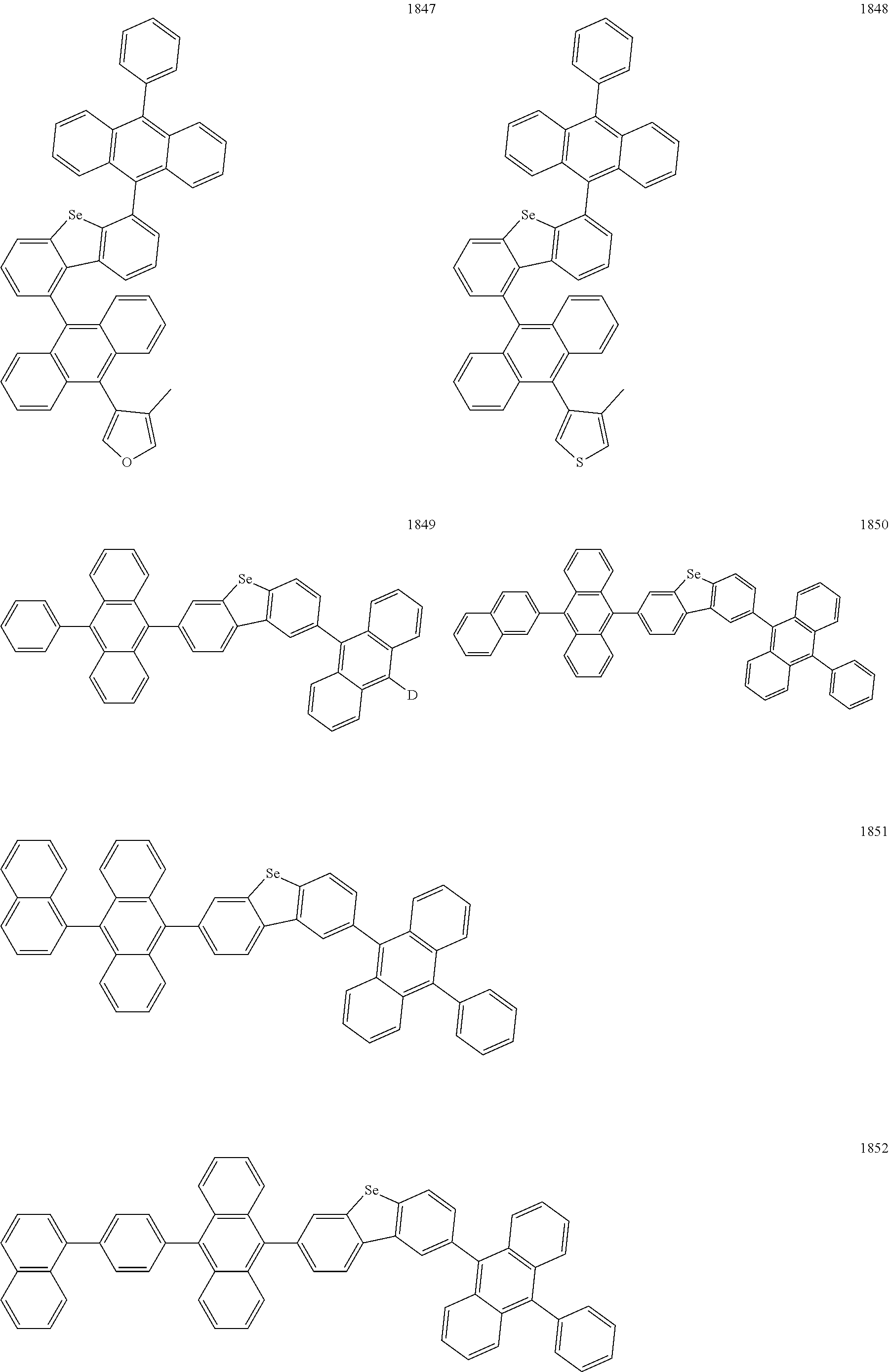

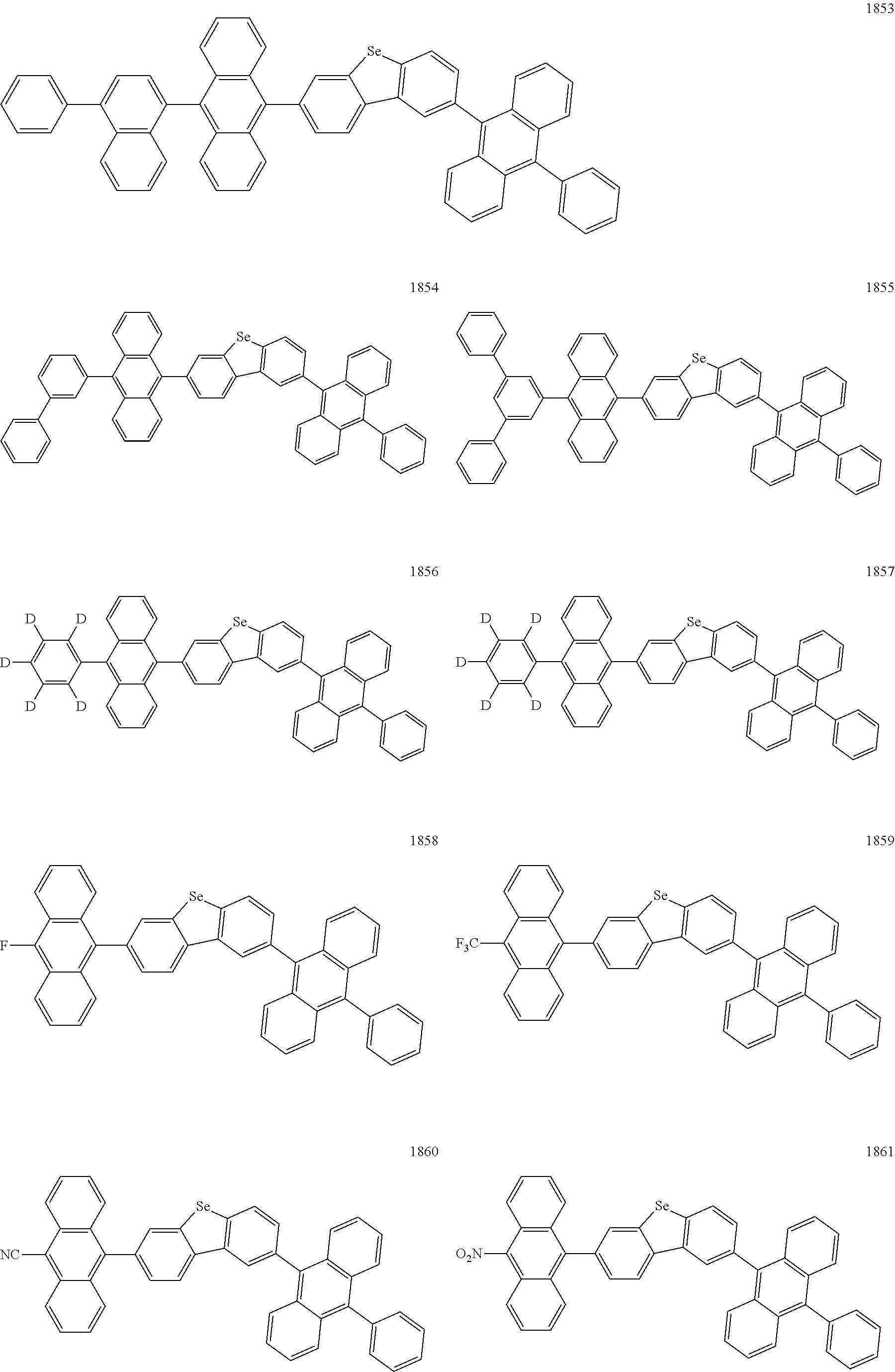

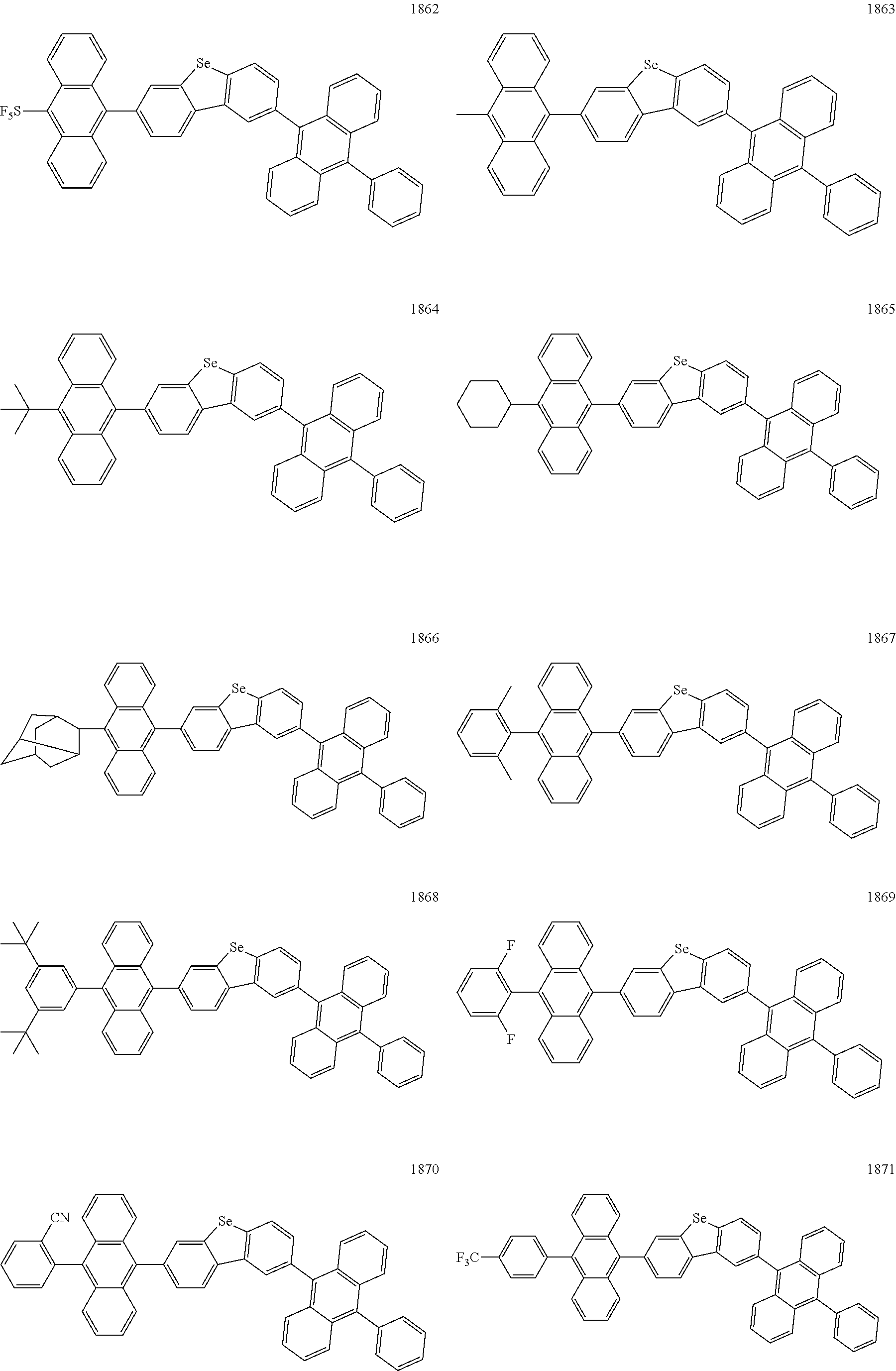

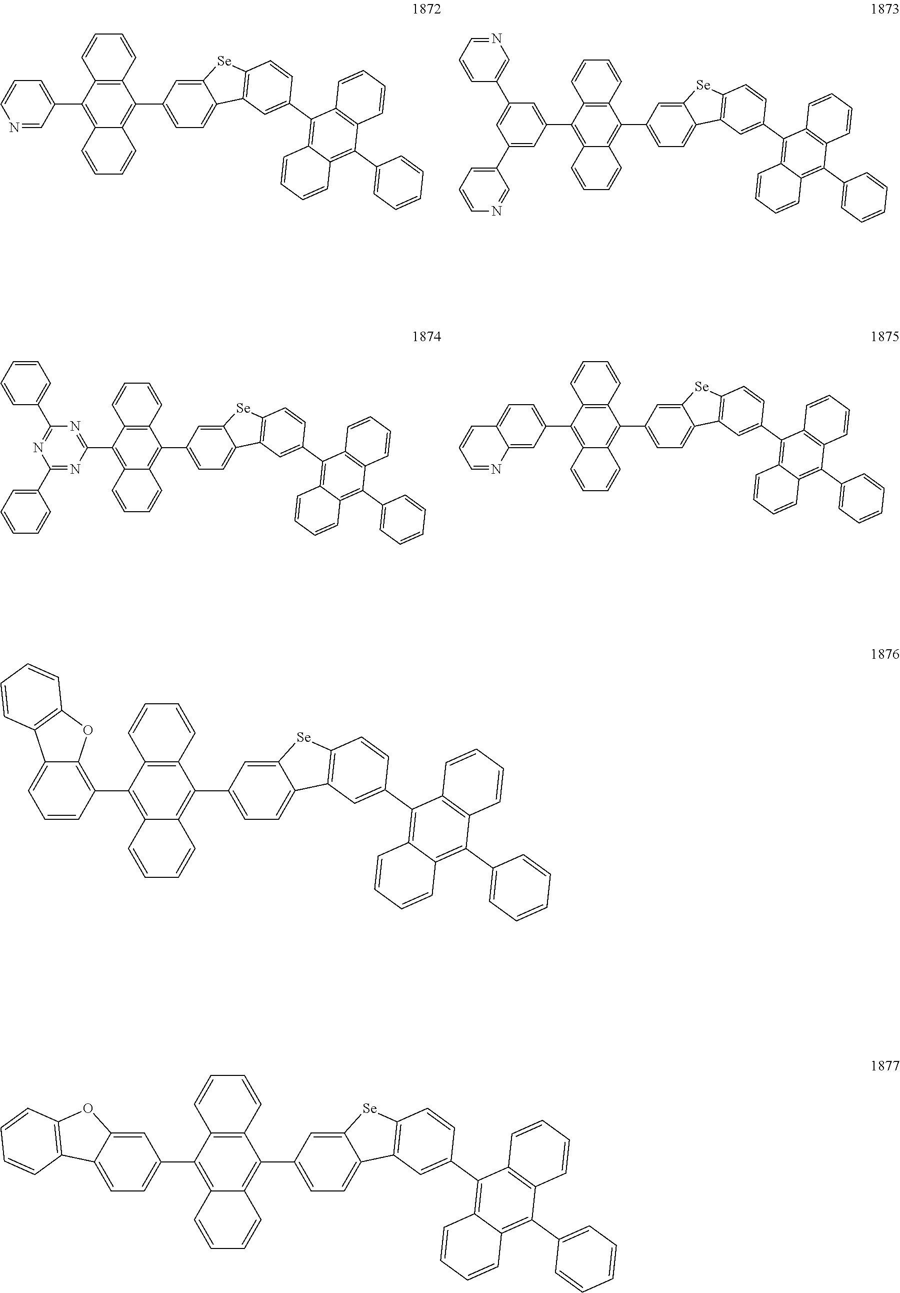

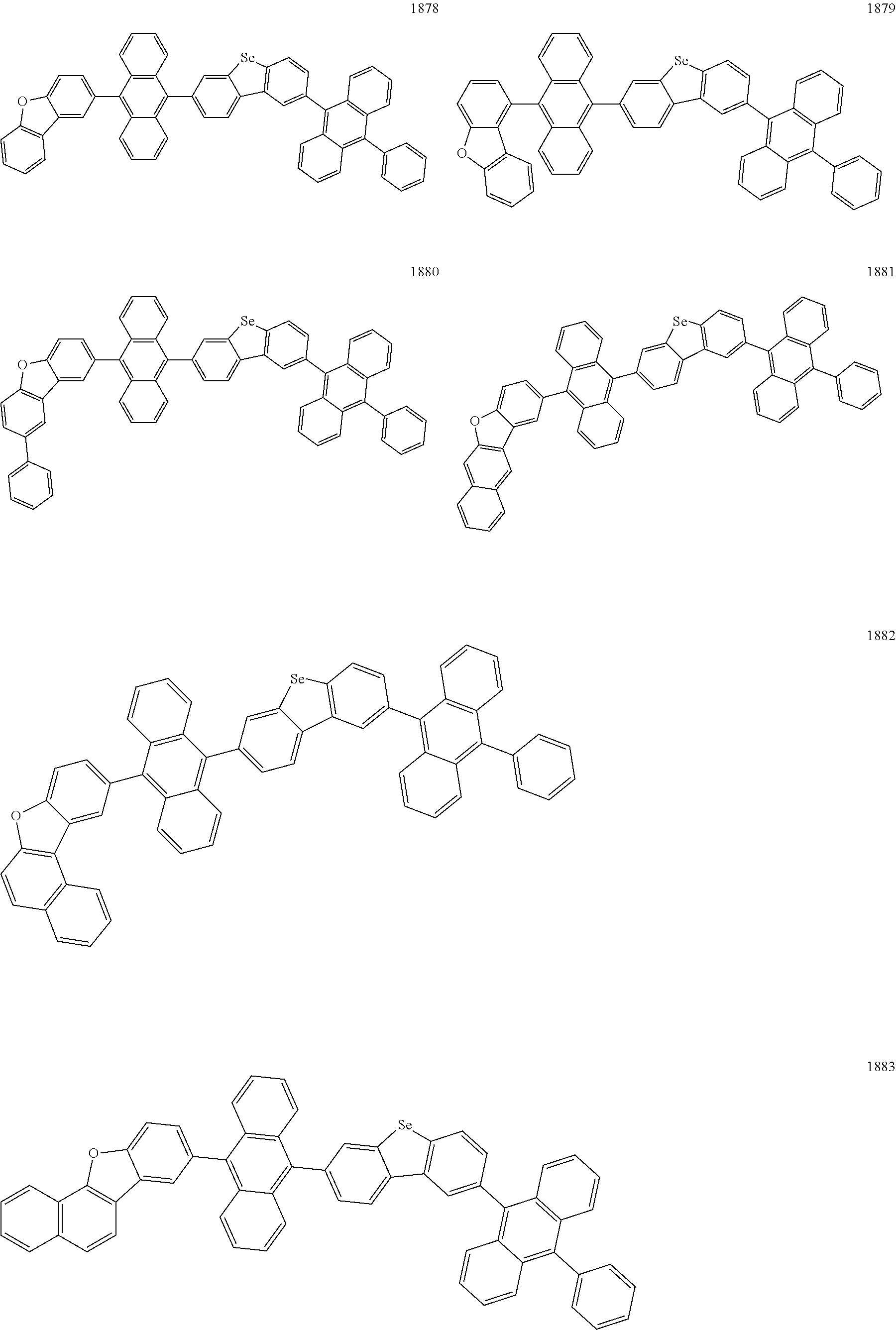

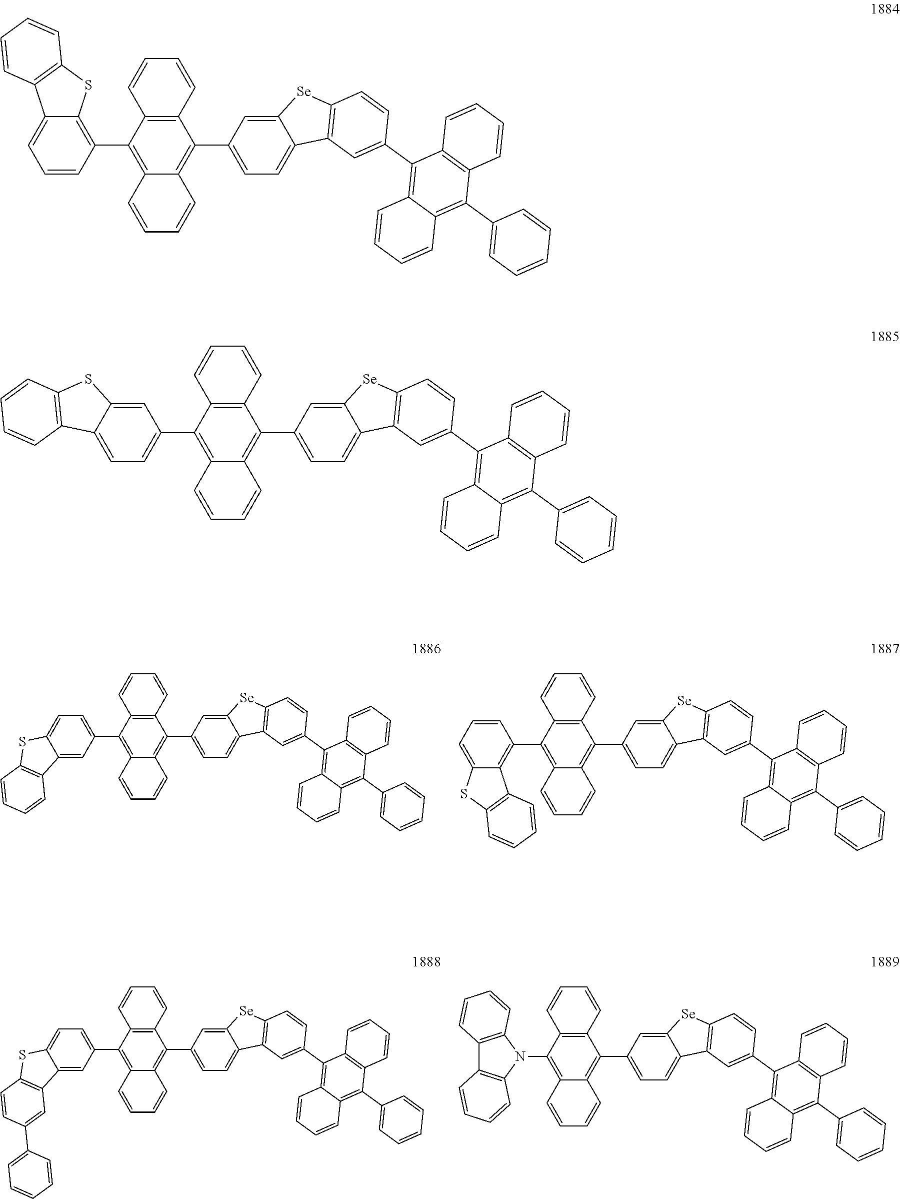

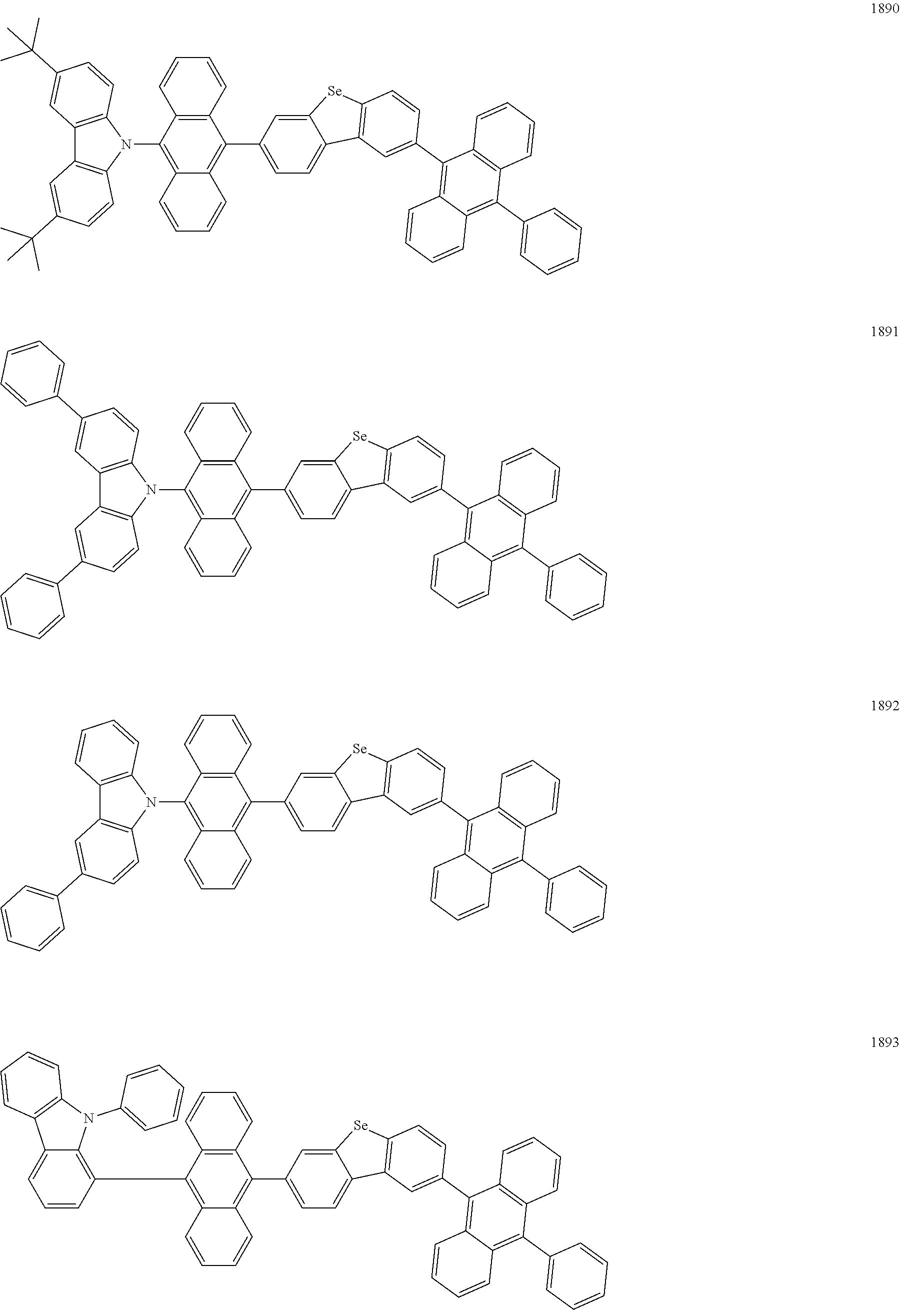 [SMILES: CC(C)(C)c1ccc2c(c1)c1cc(C(C)(C)C)ccc1n2-c1c2ccccc2c(-c2ccc3c(c2)[se]c2ccc(-c4c5ccccc5c(-c5ccccc5)c5ccccc45)cc23)c2ccccc12 ; c1ccc(-c2ccc3c(c2)c2cc(-c4ccccc4)ccc2n3-c2c3ccccc3c(-c3ccc4c(c3)[se]c3ccc(-c5c6ccccc6c(-c6ccccc6)c6ccccc56)cc34)c3ccccc23)cc1 ; c1ccc(-c2ccc3c(c2)c2ccccc2n3-c2c3ccccc3c(-c3ccc4c(c3)[se]c3ccc(-c5c6ccccc6c(-c6ccccc6)c6ccccc56)cc34)c3ccccc23)cc1 ; c1ccc(-c2c3ccccc3c(-c3ccc4[se]c5cc(-c6c7ccccc7c(-c7cccc8c9ccccc9n(-c9ccccc9)c78)c7ccccc67)ccc5c4c3)c3ccccc23)cc1]

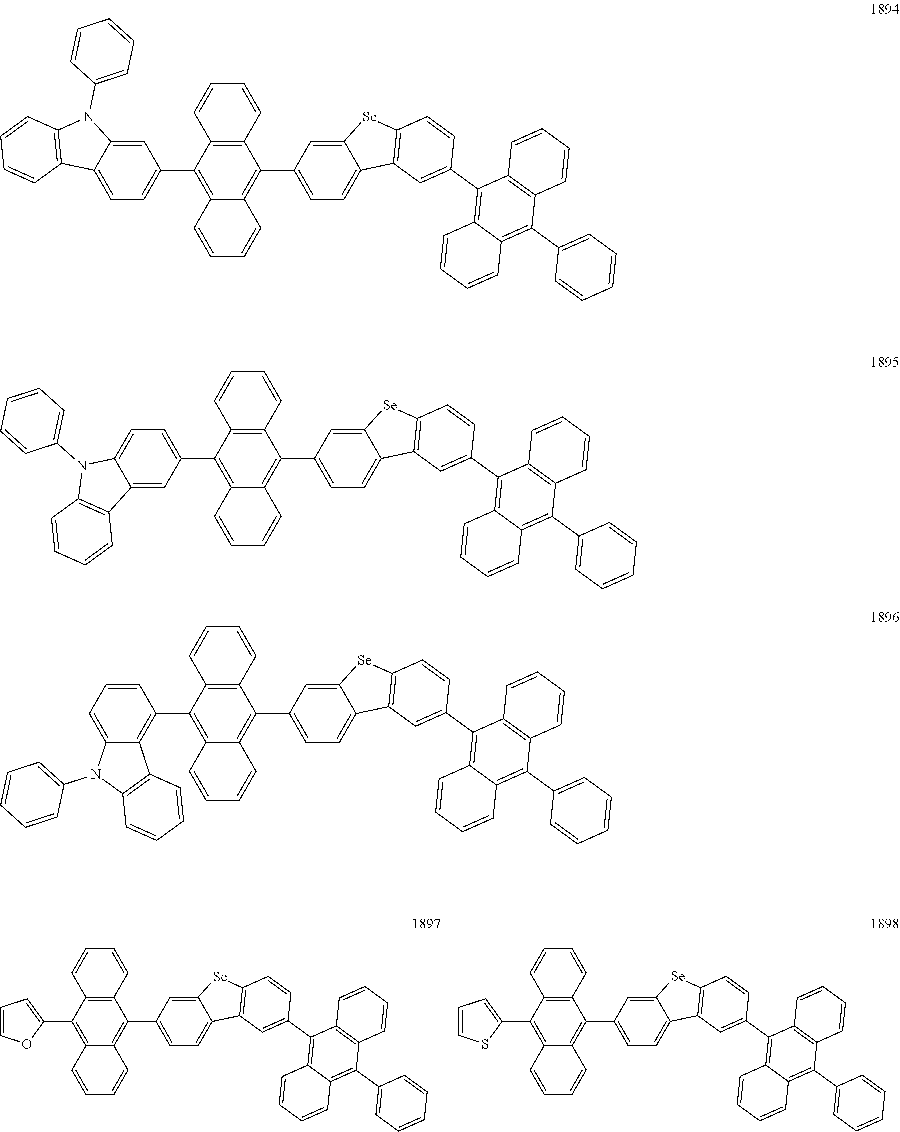

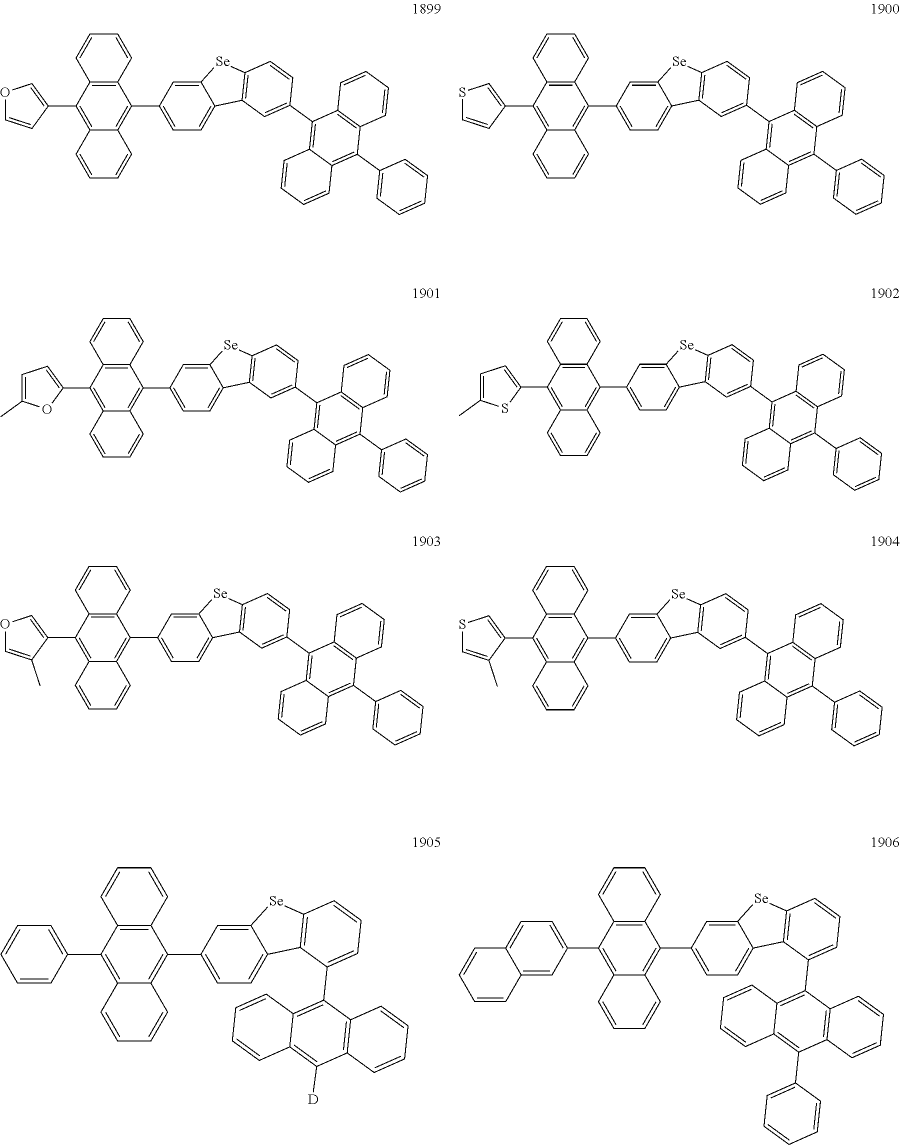

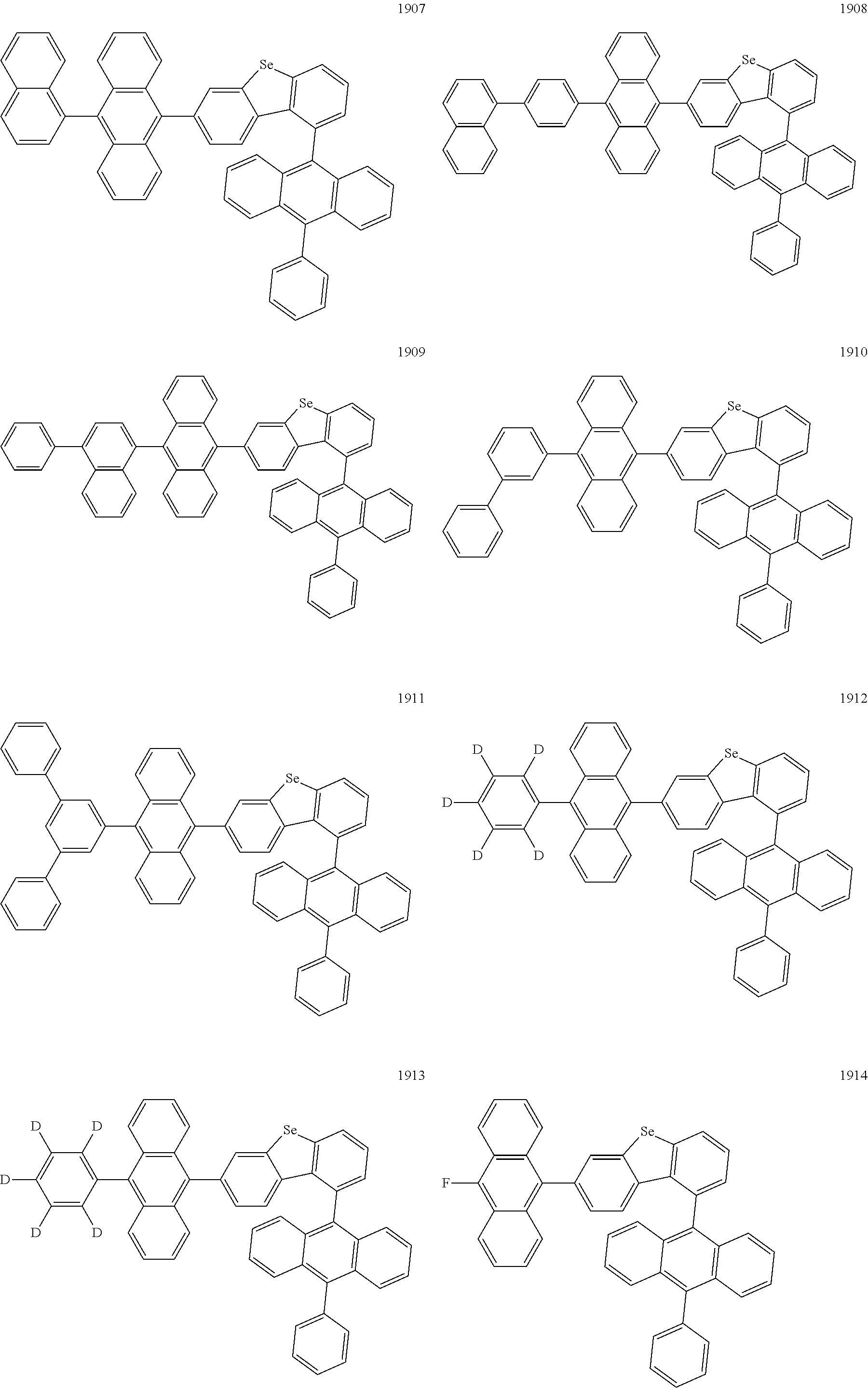

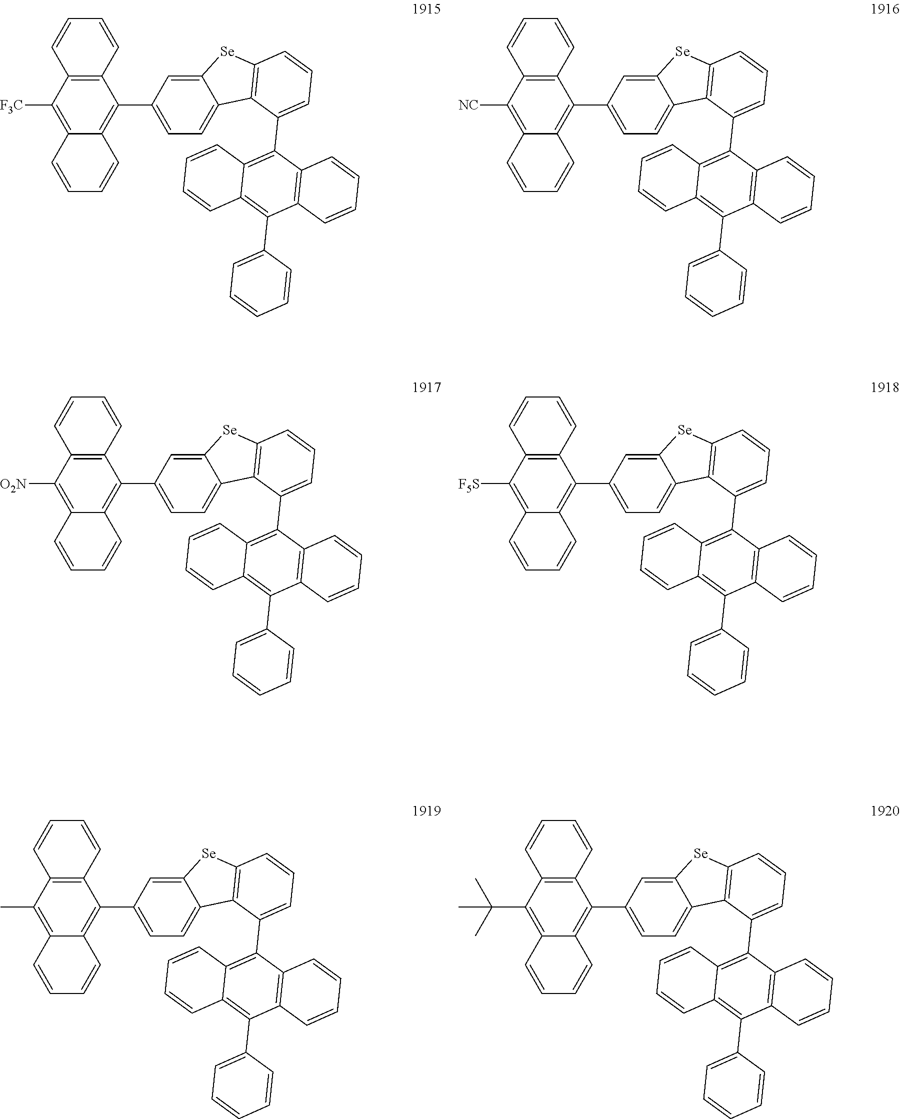

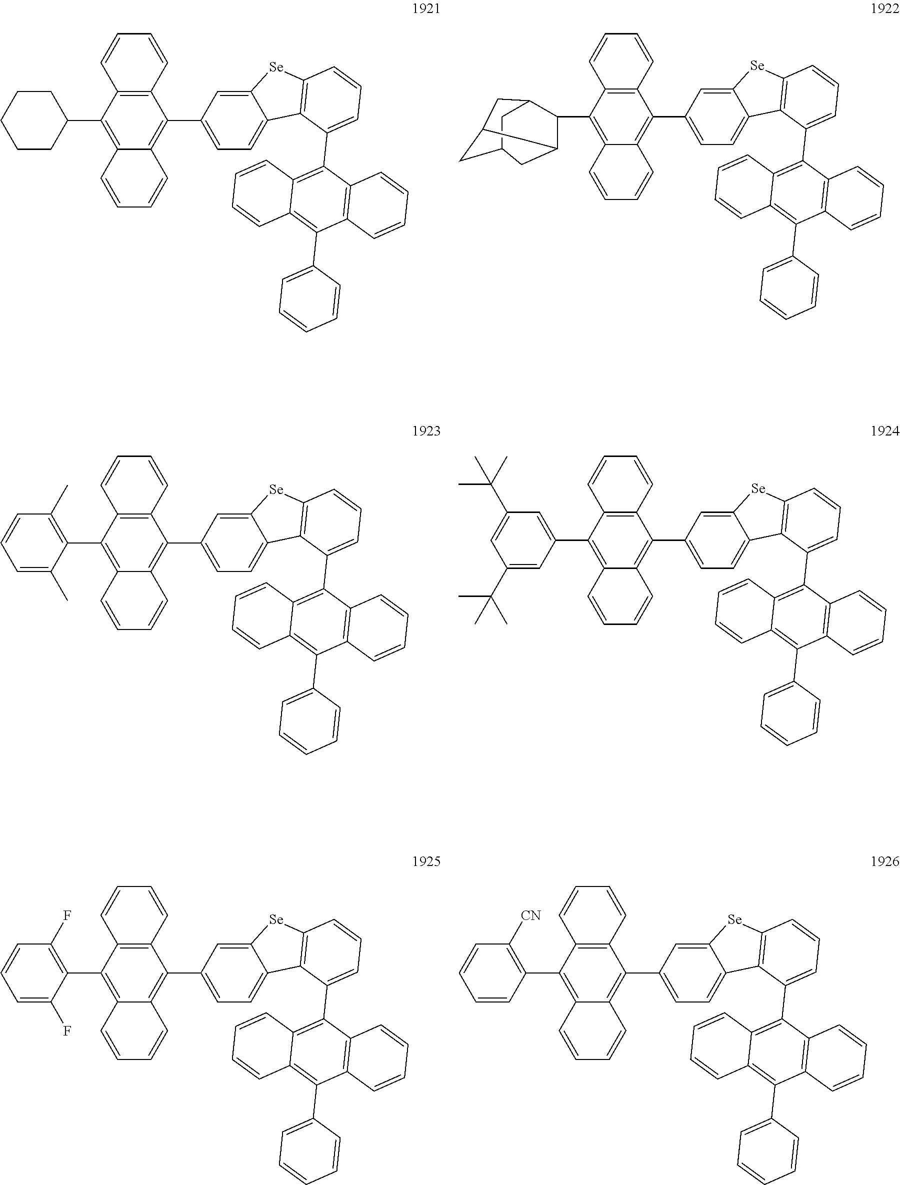

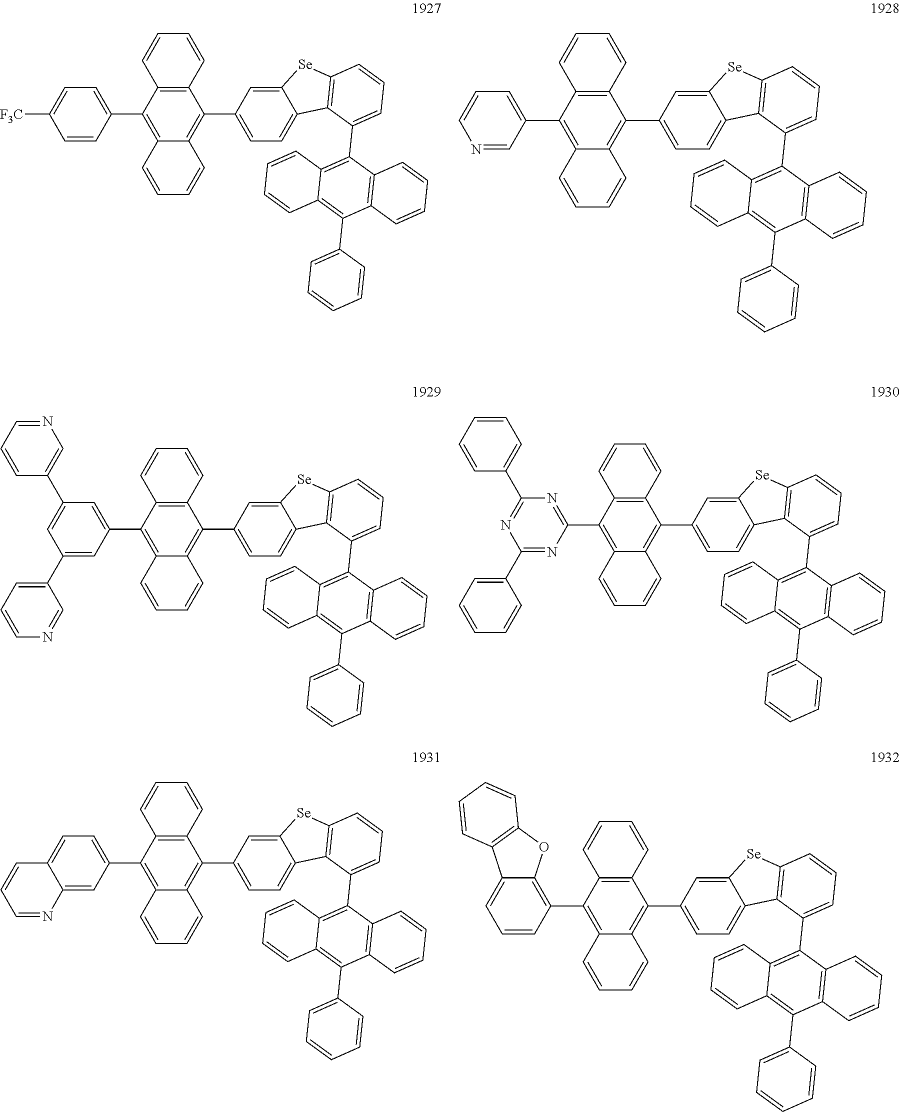

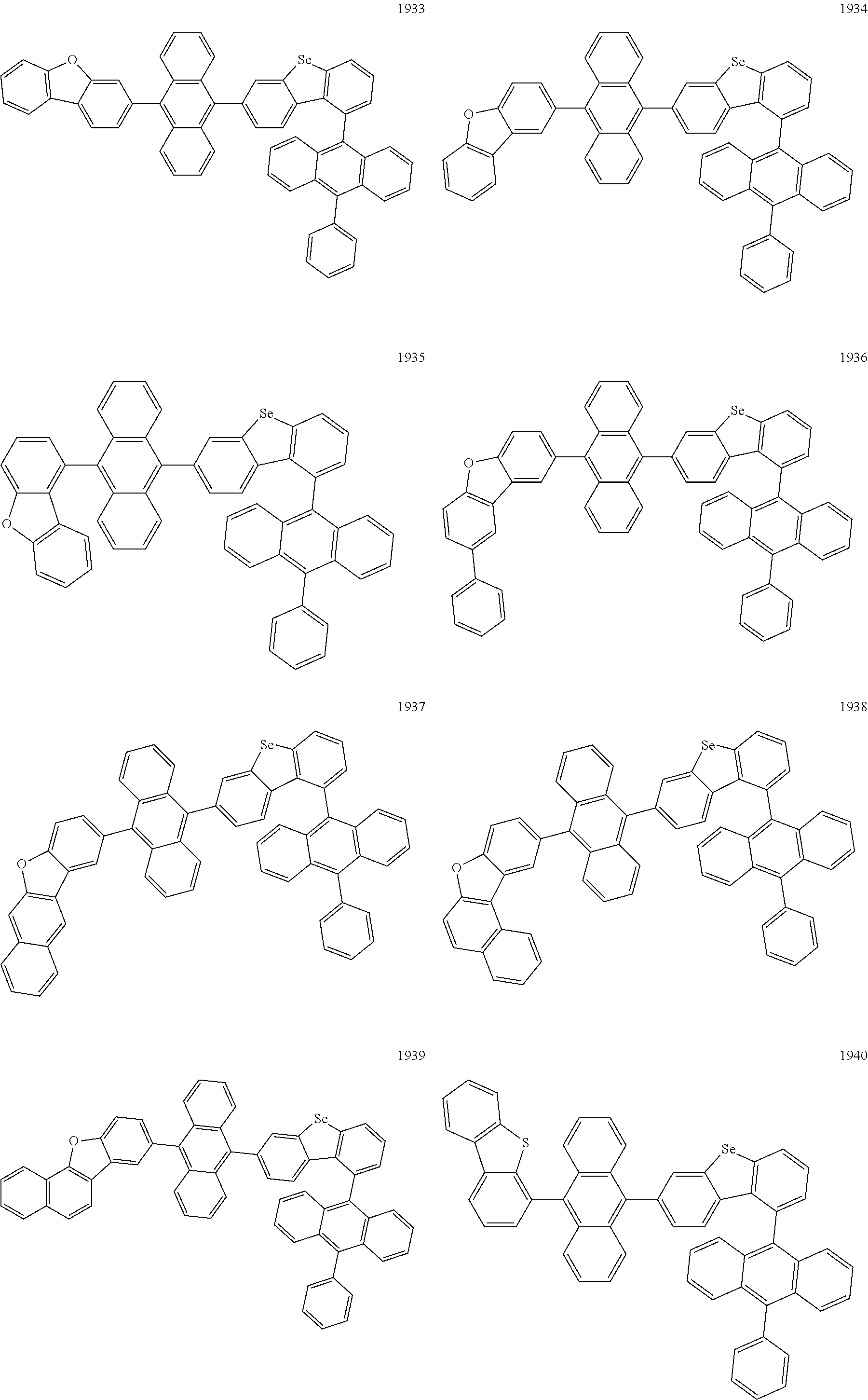

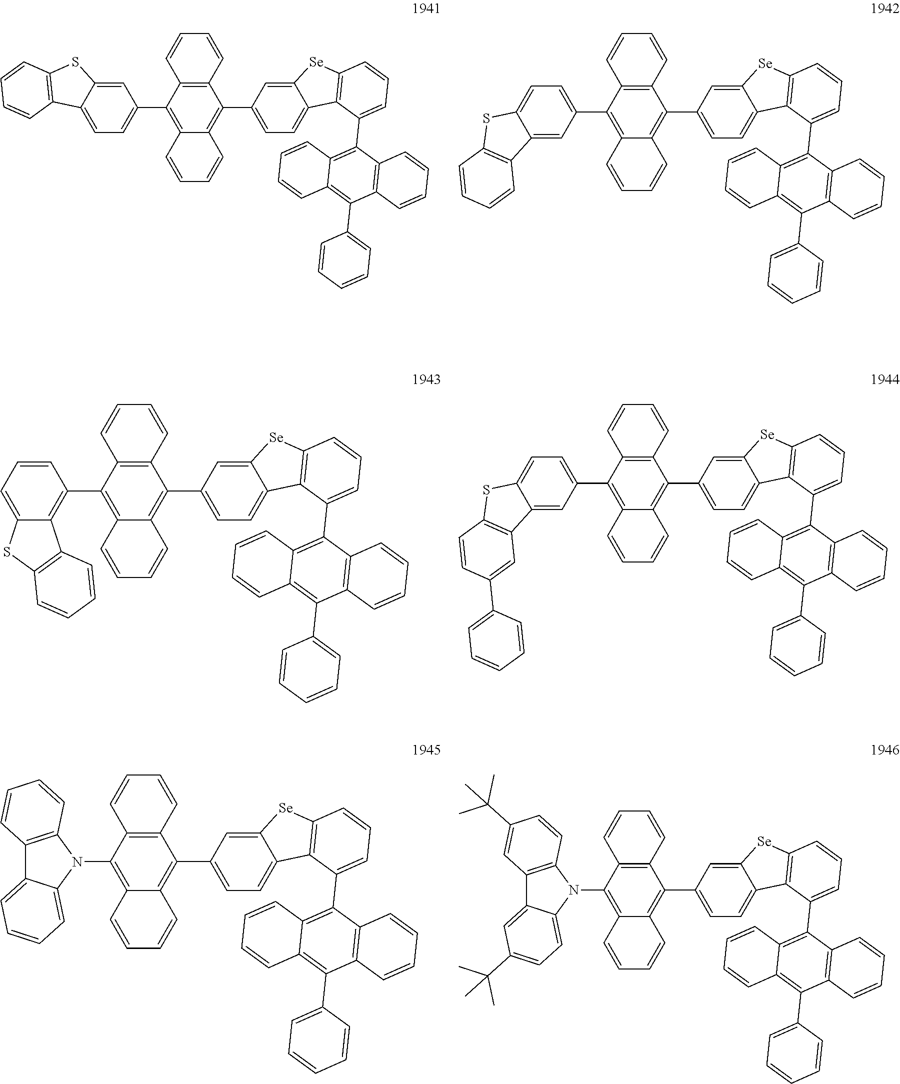

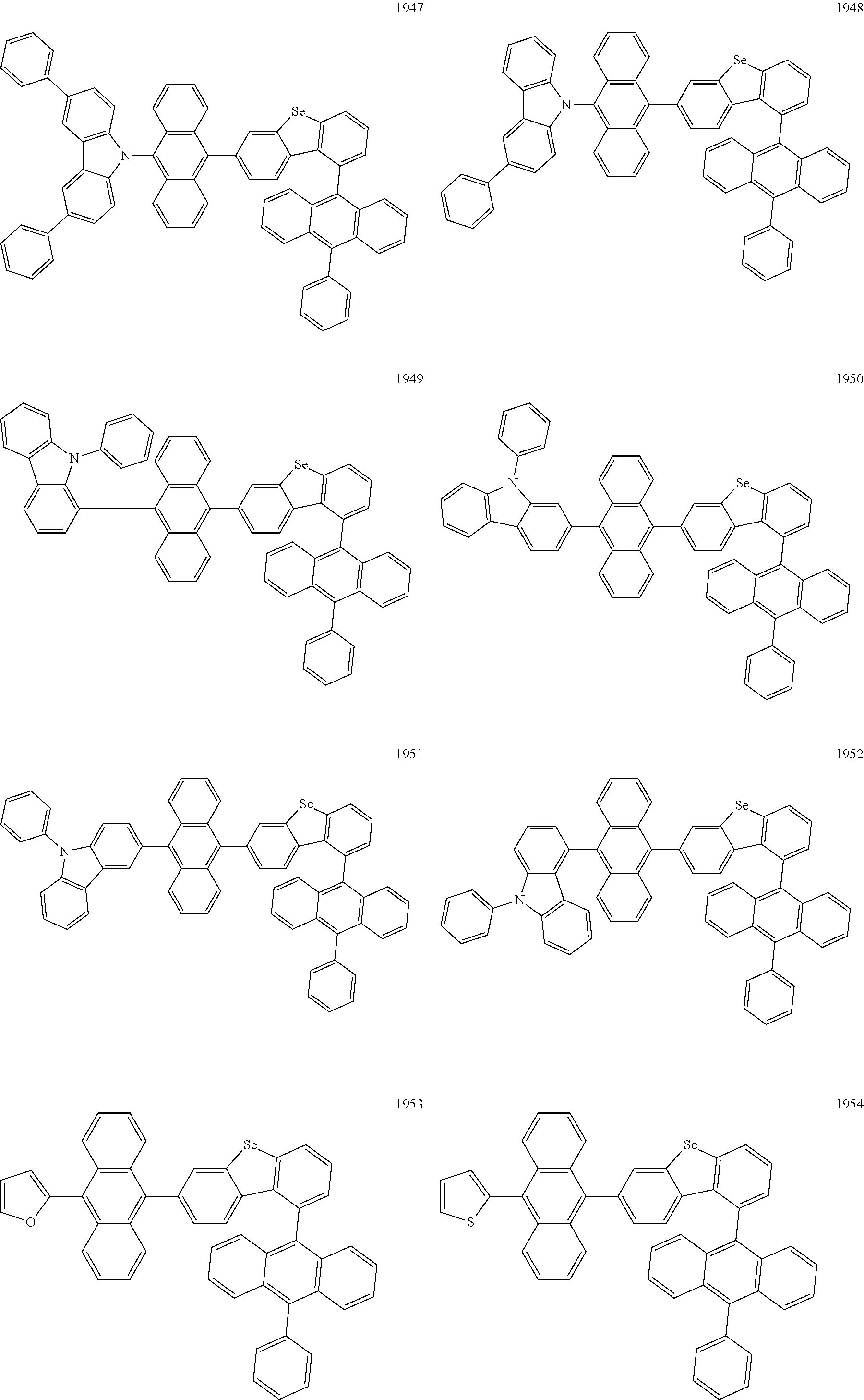

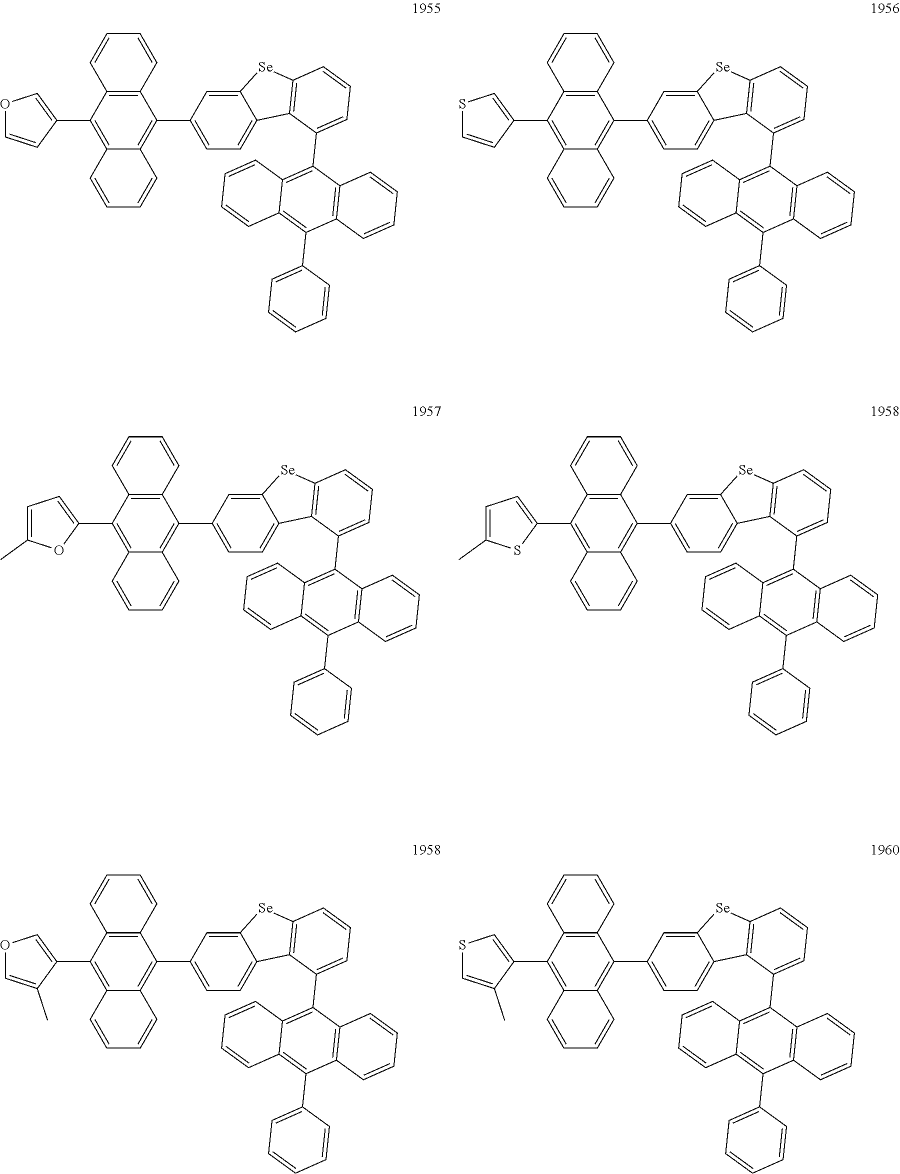

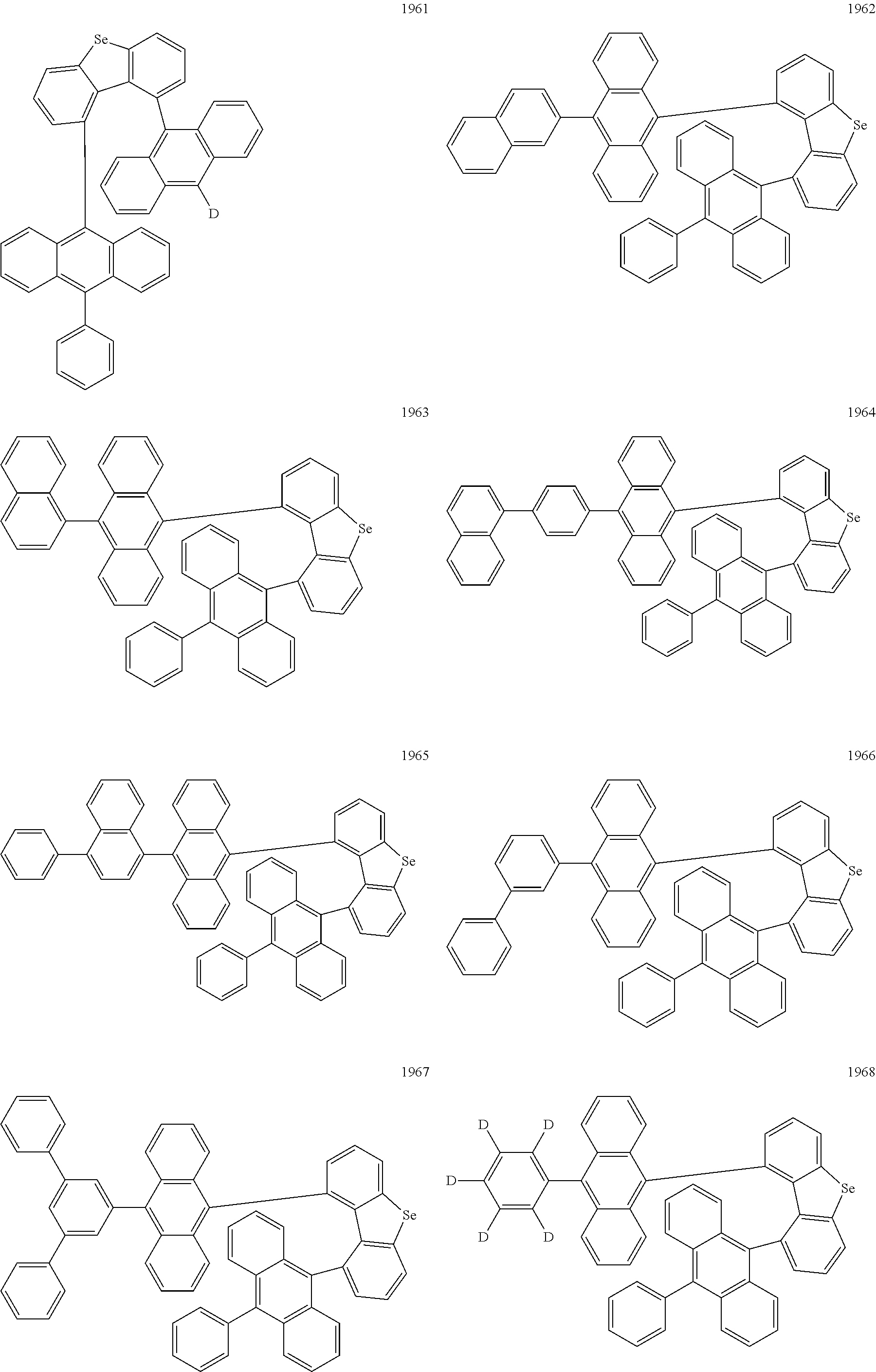

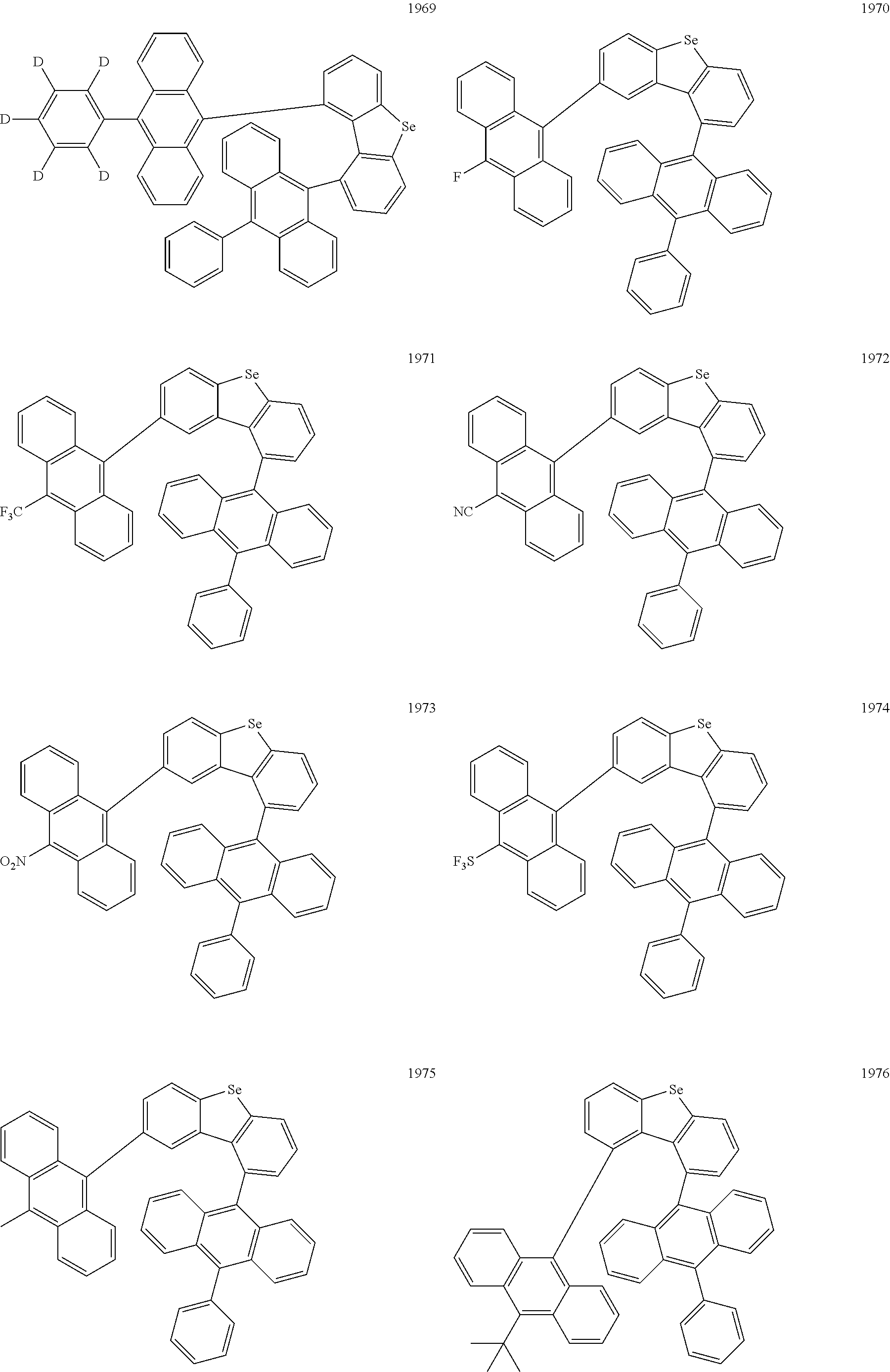

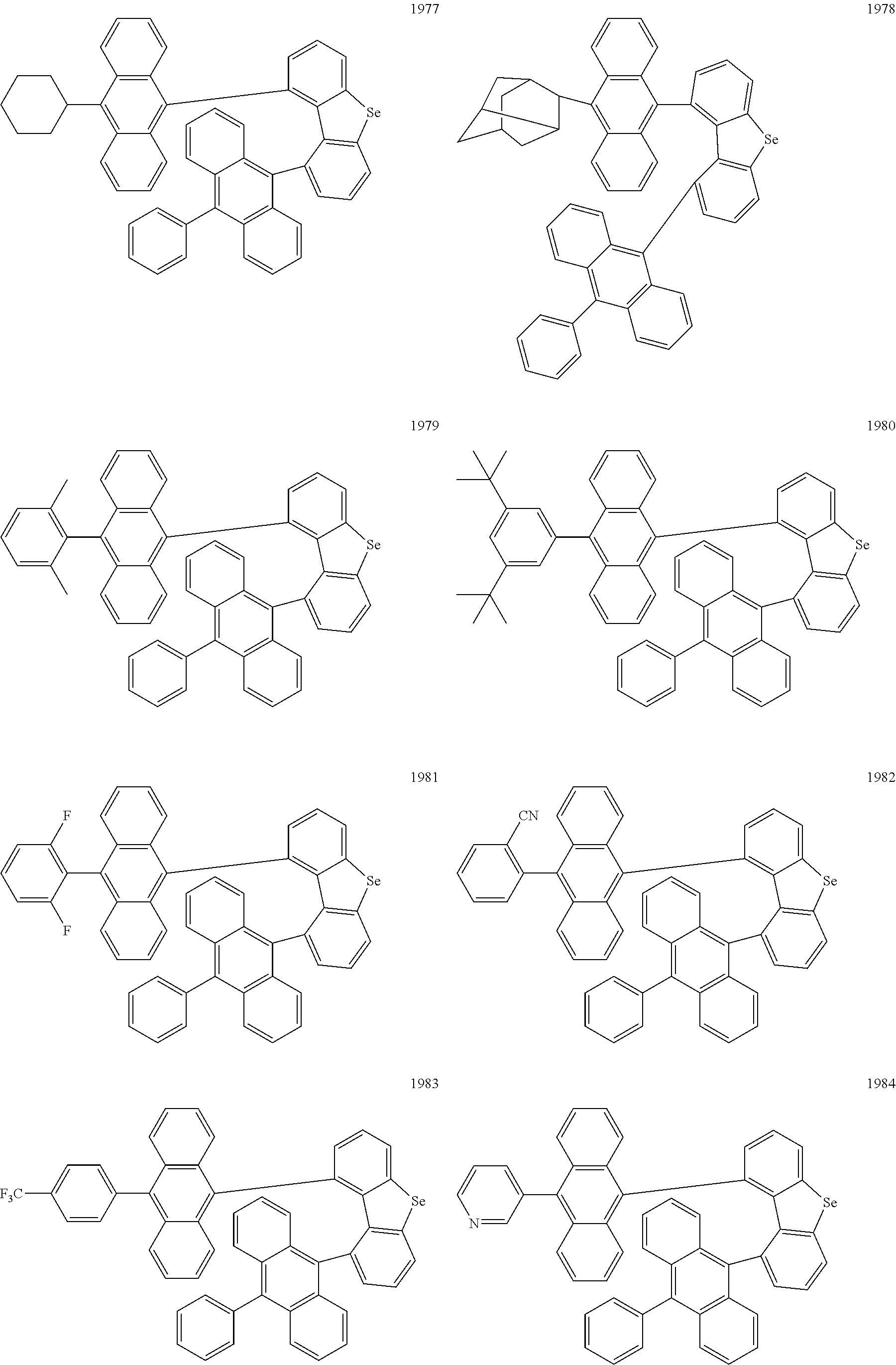

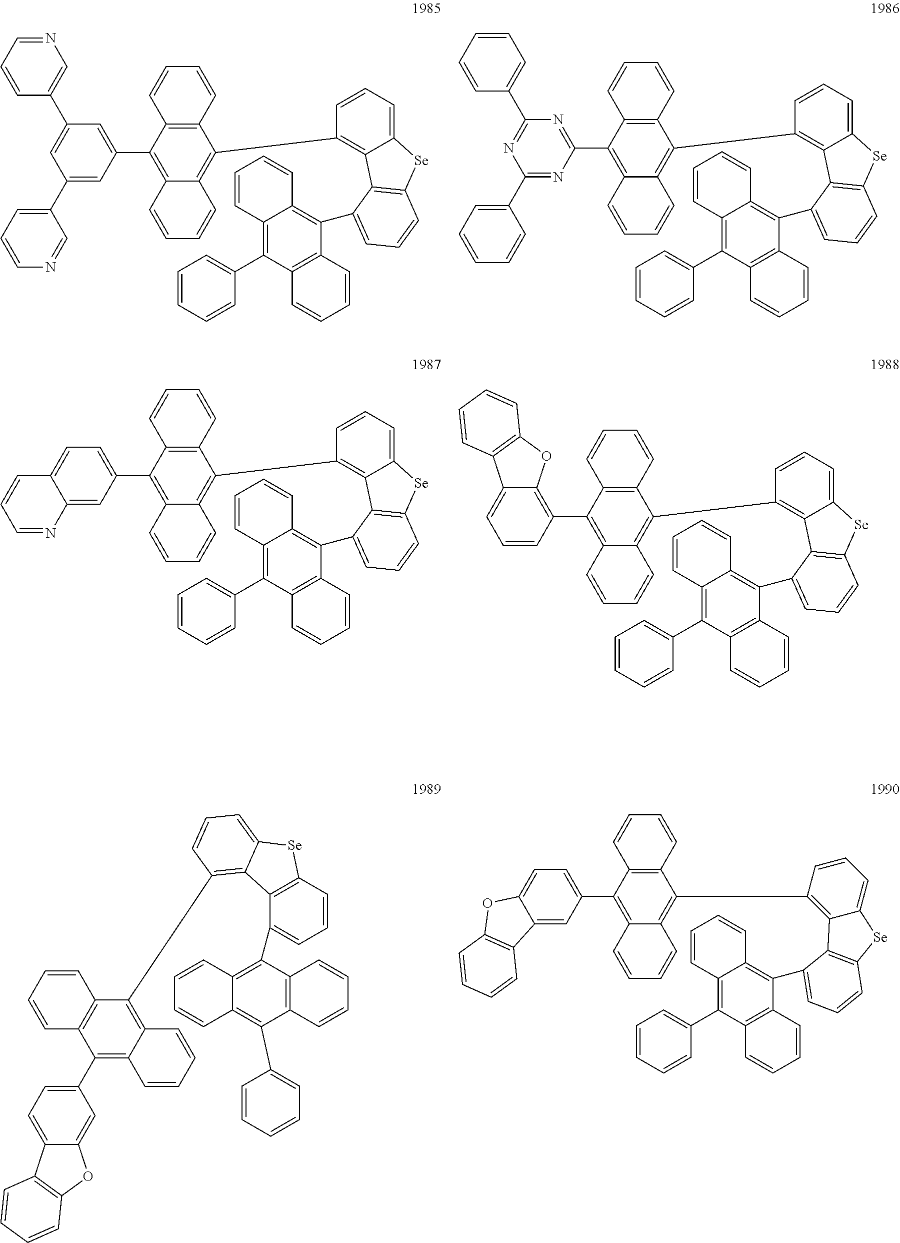

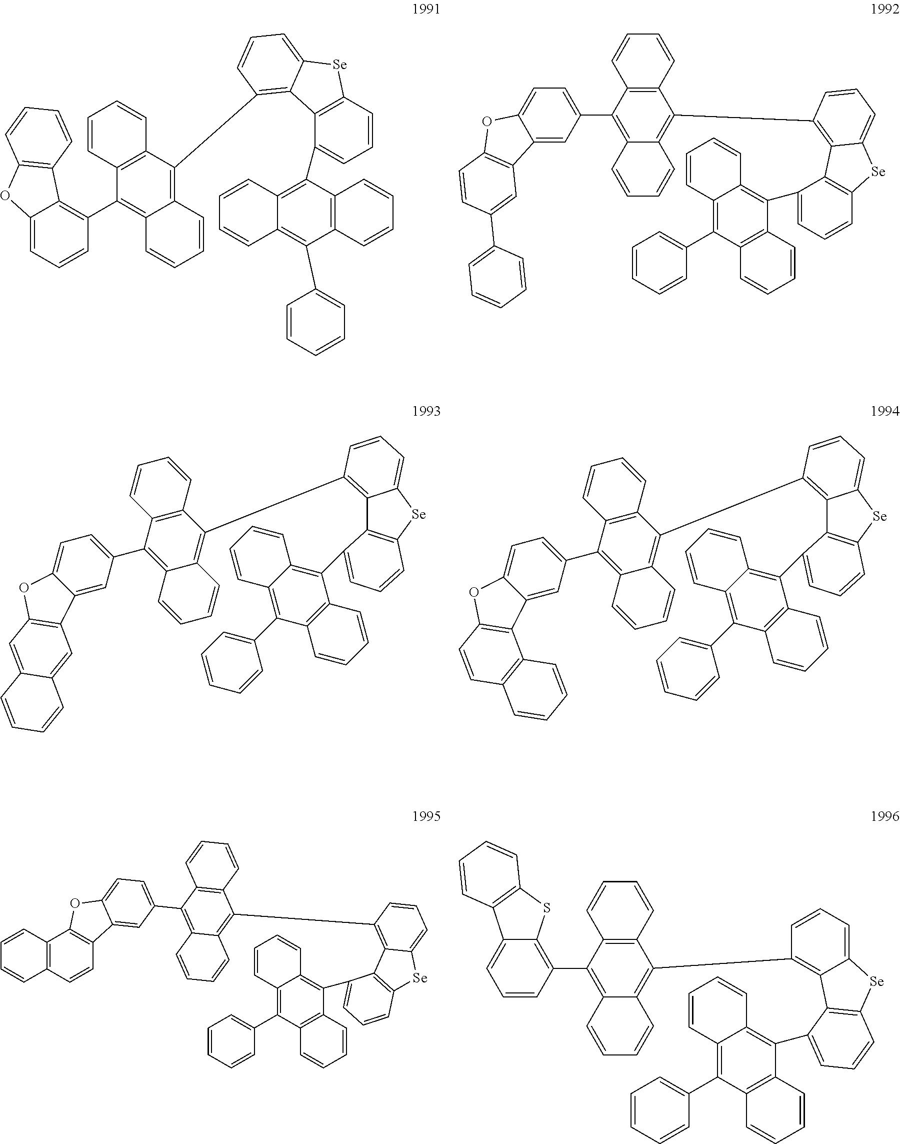

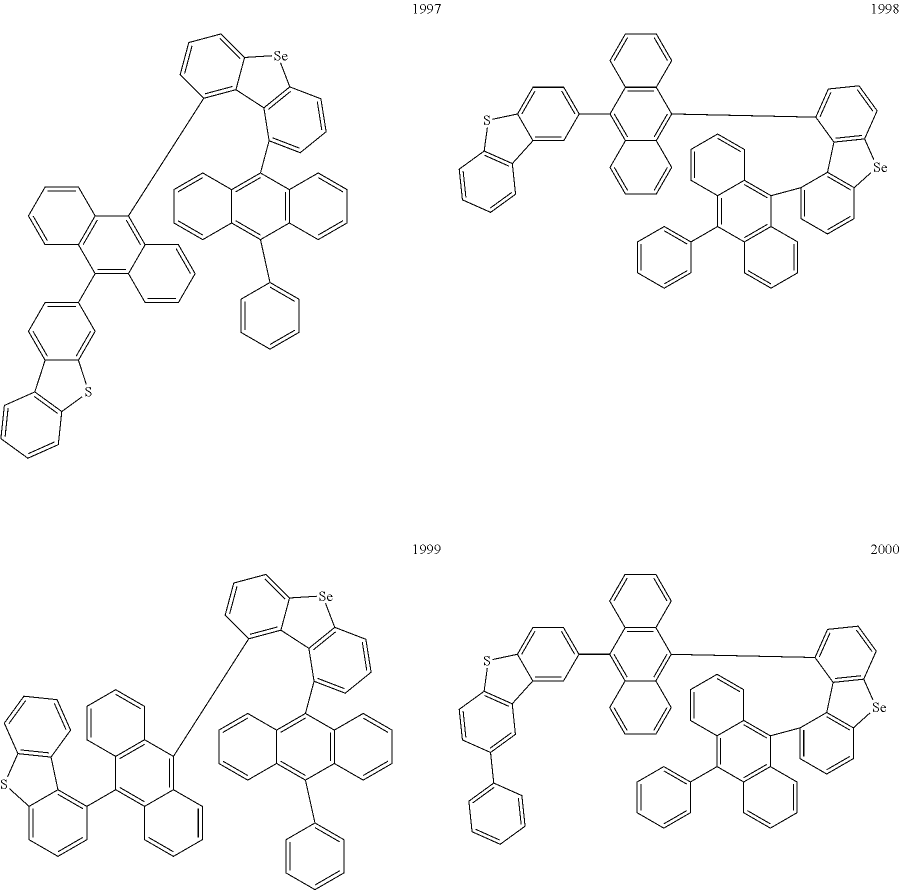 [SMILES: c1ccc(-c2c3ccccc3c(-c3cccc4[se]c5cccc(-c6c7ccccc7c(-c7ccc8c(c7)sc7ccccc78)c7ccccc67)c5c34)c3ccccc23)cc1 ; c1ccc(-c2c3ccccc3c(-c3cccc4[se]c5cccc(-c6c7ccccc7c(-c7ccc8sc9ccccc9c8c7)c7ccccc67)c5c34)c3ccccc23)cc1 ; c1ccc(-c2c3ccccc3c(-c3cccc4[se]c5cccc(-c6c7ccccc7c(-c7cccc8sc9ccccc9c78)c7ccccc67)c5c34)c3ccccc23)cc1 ; c1ccc(-c2ccc3sc4ccc(-c5c6ccccc6c(-c6cccc7[se]c8cccc(-c9c%10ccccc%10c(-c%10ccccc%10)c%10ccccc9%10)c8c67)c6ccccc56)cc4c3c2)cc1]

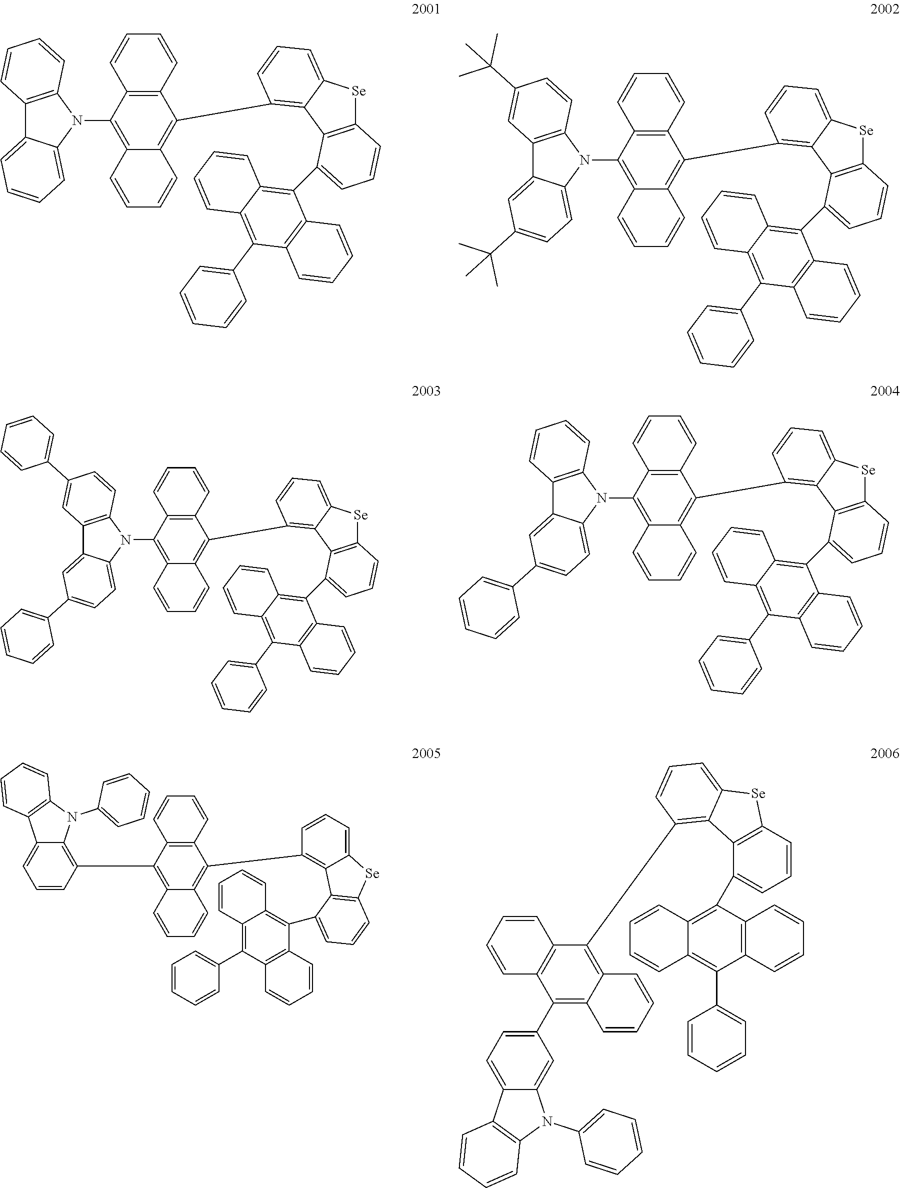

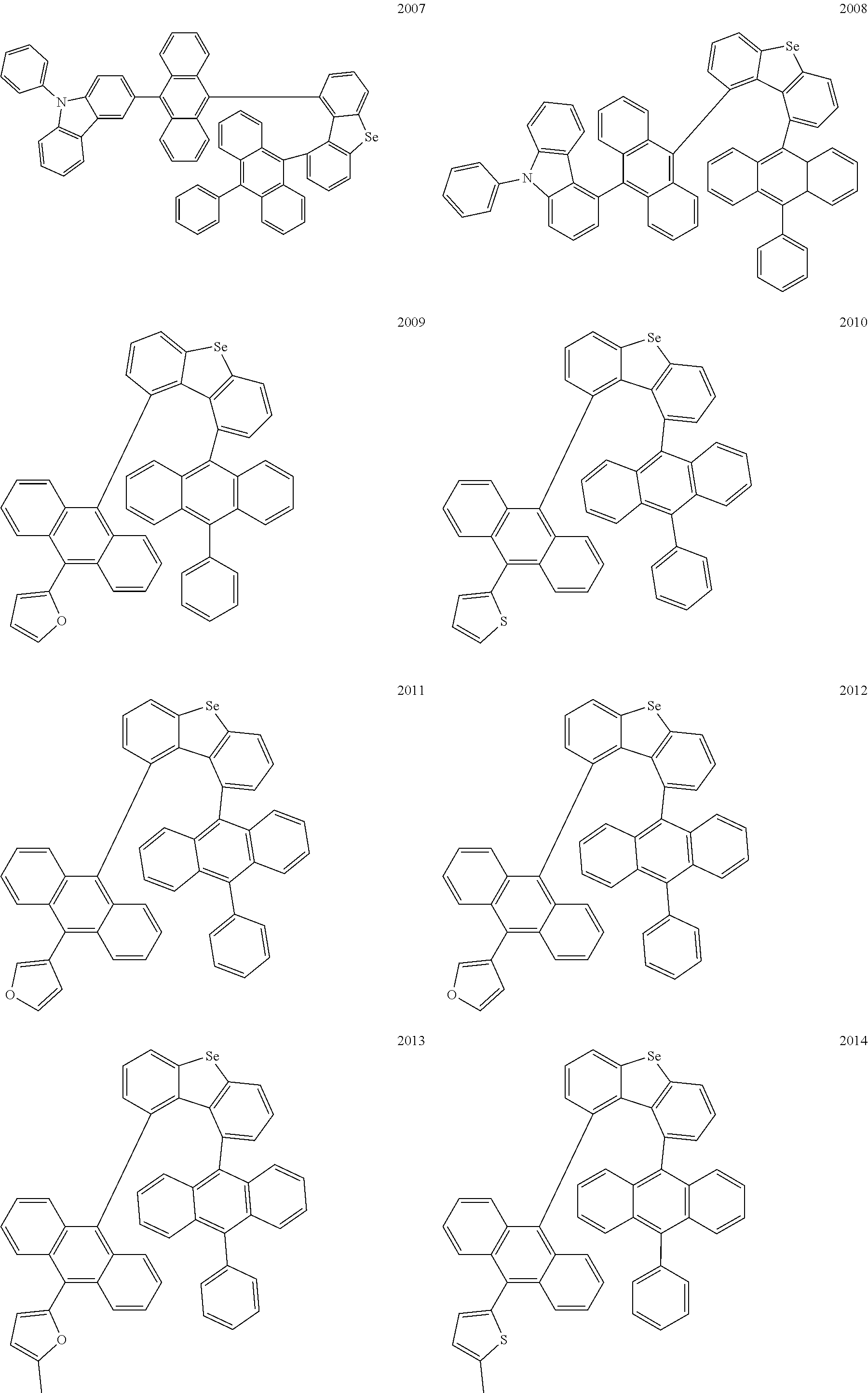

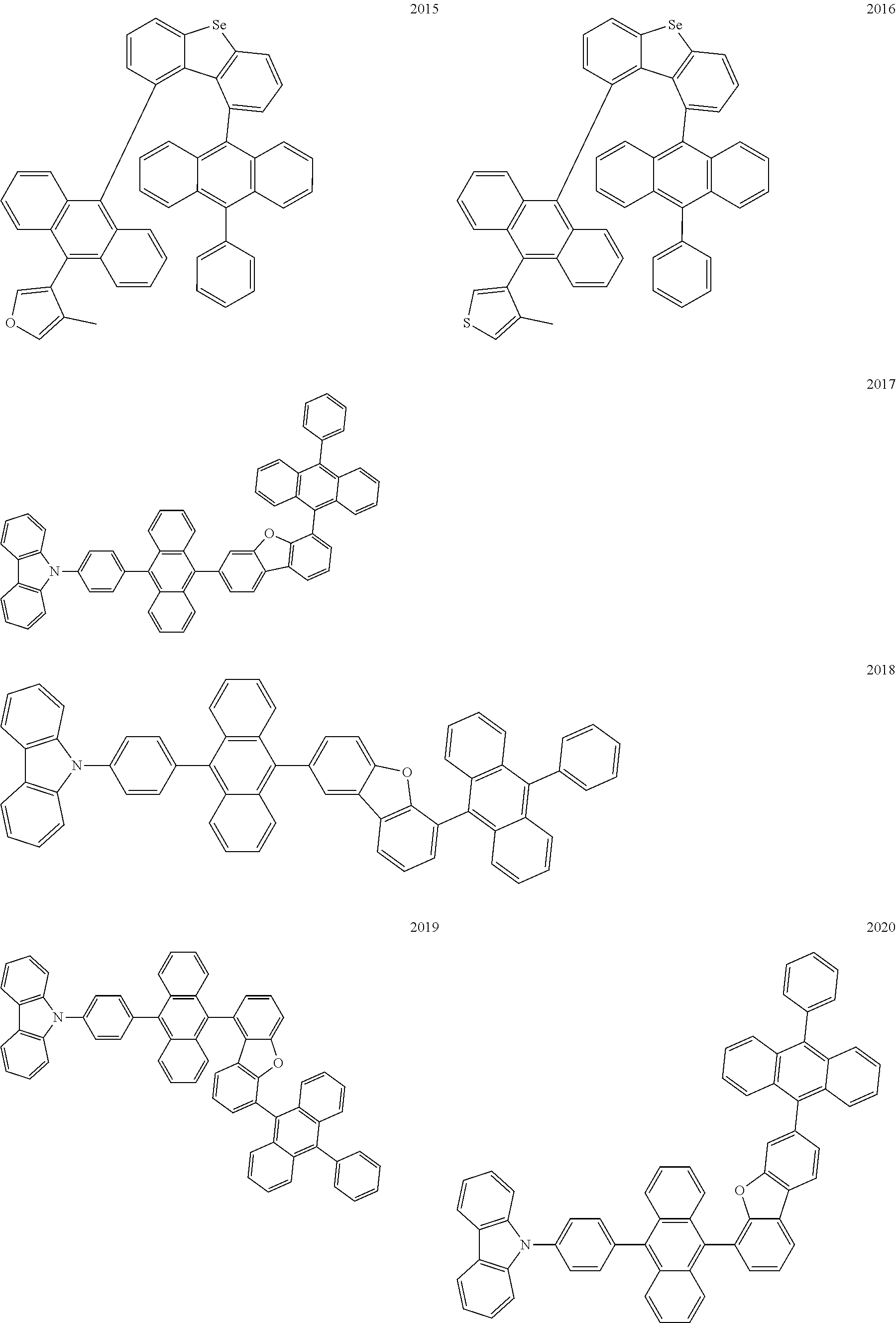

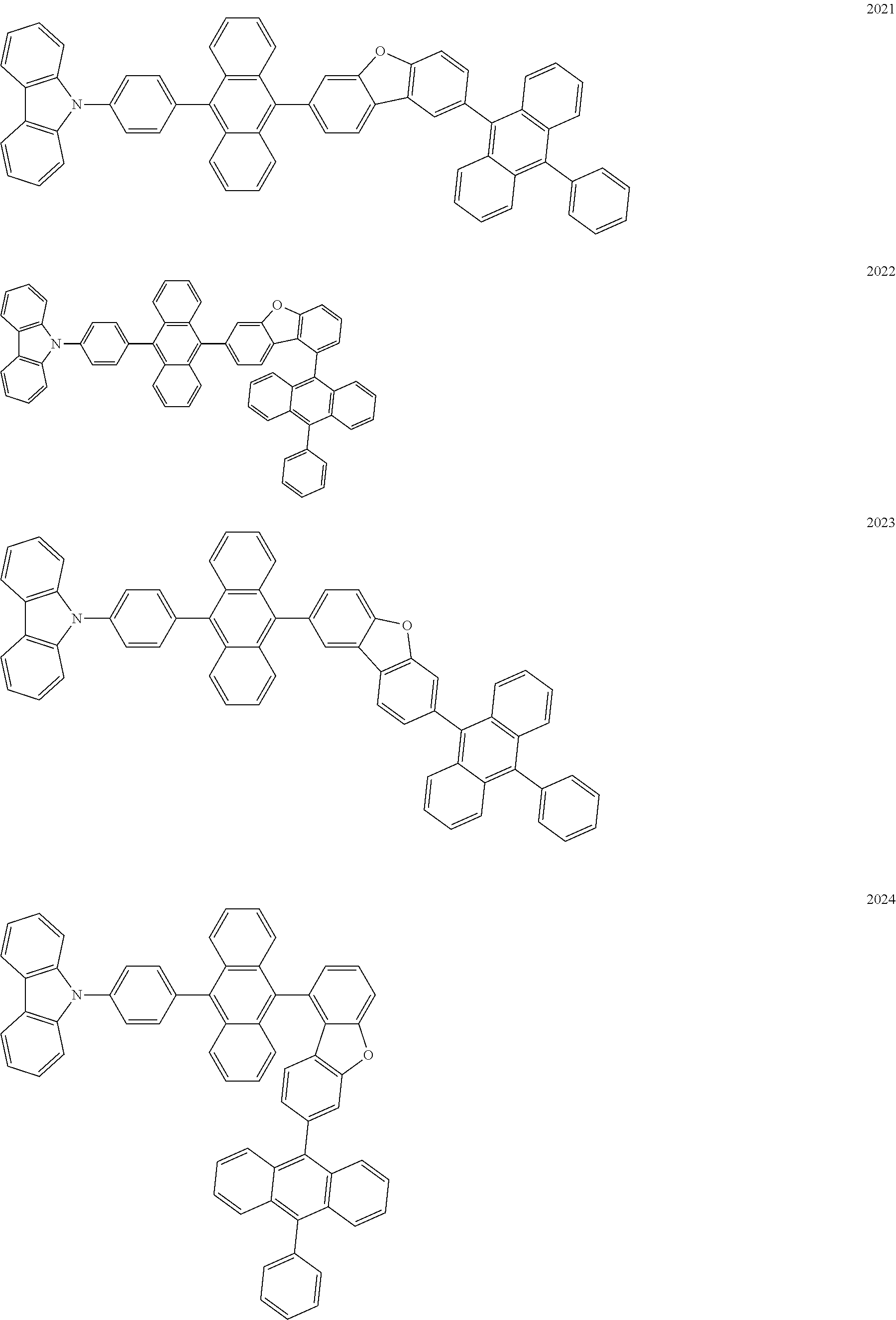 [SMILES: c1ccc(-c2c3ccccc3c(-c3ccc4oc5cc(-c6c7ccccc7c(-c7ccc(-n8c9ccccc9c9ccccc98)cc7)c7ccccc67)ccc5c4c3)c3ccccc23)cc1 ; c1ccc(-c2c3ccccc3c(-c3cccc4oc5cc(-c6c7ccccc7c(-c7ccc(-n8c9ccccc9c9ccccc98)cc7)c7ccccc67)ccc5c34)c3ccccc23)cc1 ; c1ccc(-c2c3ccccc3c(-c3ccc4c(c3)oc3ccc(-c5c6ccccc6c(-c6ccc(-n7c8ccccc8c8ccccc87)cc6)c6ccccc56)cc34)c3ccccc23)cc1 ; c1ccc(-c2c3ccccc3c(-c3ccc4c(c3)oc3cccc(-c5c6ccccc6c(-c6ccc(-n7c8ccccc8c8ccccc87)cc6)c6ccccc56)c34)c3ccccc23)cc1]

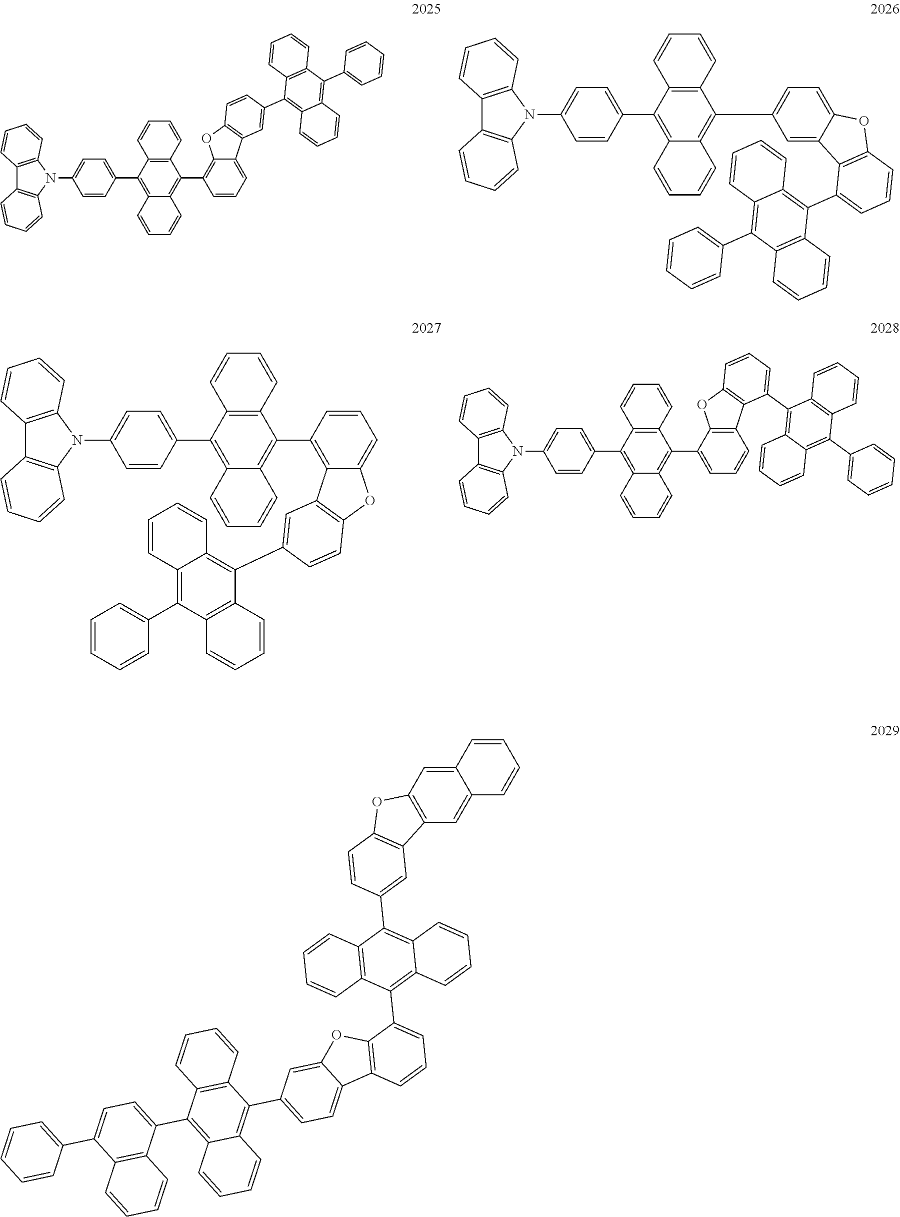

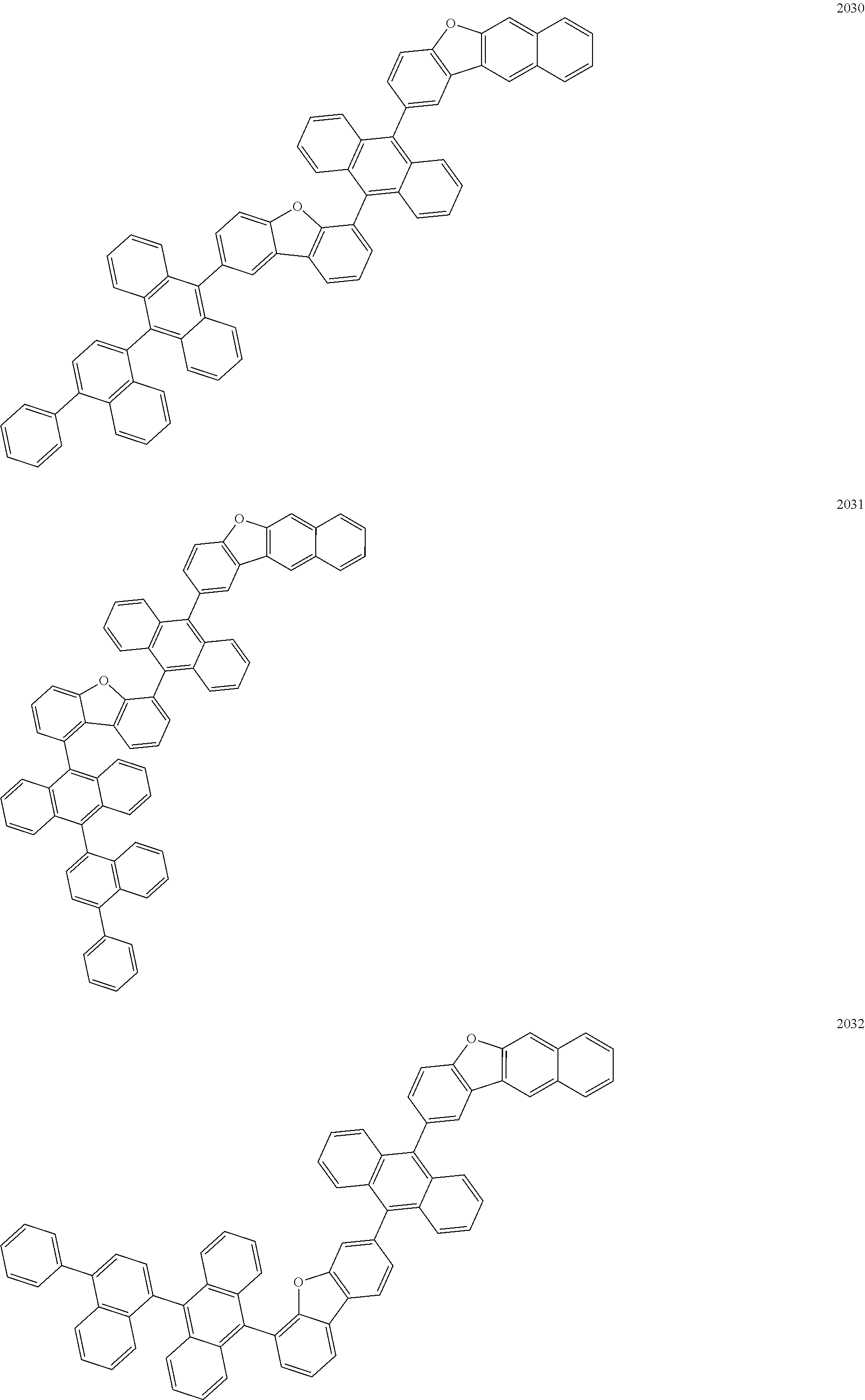 [SMILES: c1ccc(-c2ccc(-c3c4ccccc4c(-c4ccc5oc6c(-c7c8ccccc8c(-c8ccc9oc%10cc%11ccccc%11cc%10c9c8)c8ccccc78)cccc6c5c4)c4ccccc34)c3ccccc23)cc1 ; c1ccc(-c2ccc(-c3c4ccccc4c(-c4cccc5oc6c(-c7c8ccccc8c(-c8ccc9oc%10cc%11ccccc%11cc%10c9c8)c8ccccc78)cccc6c45)c4ccccc34)c3ccccc23)cc1 ; c1ccc(-c2ccc(-c3c4ccccc4c(-c4cccc5c4oc4cc(-c6c7ccccc7c(-c7ccc8oc9cc%10ccccc%10cc9c8c7)c7ccccc67)ccc45)c4ccccc34)c3ccccc23)cc1]

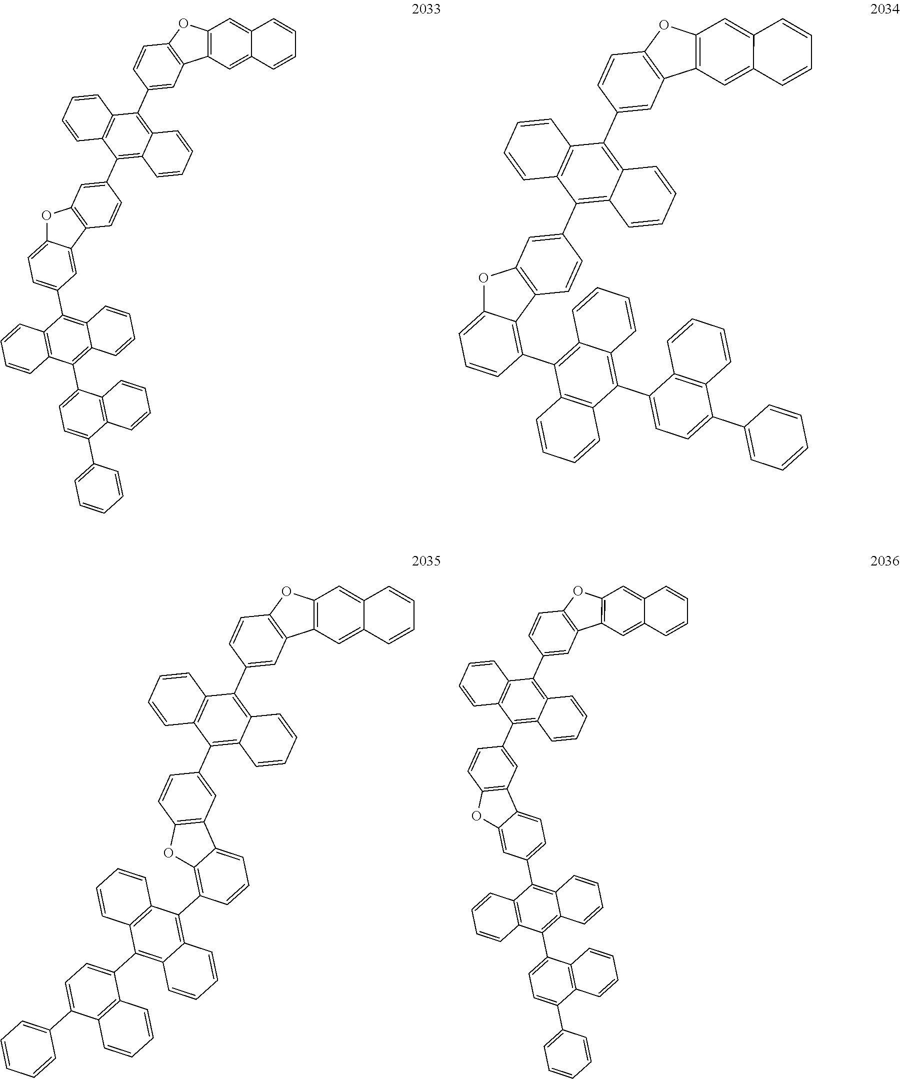

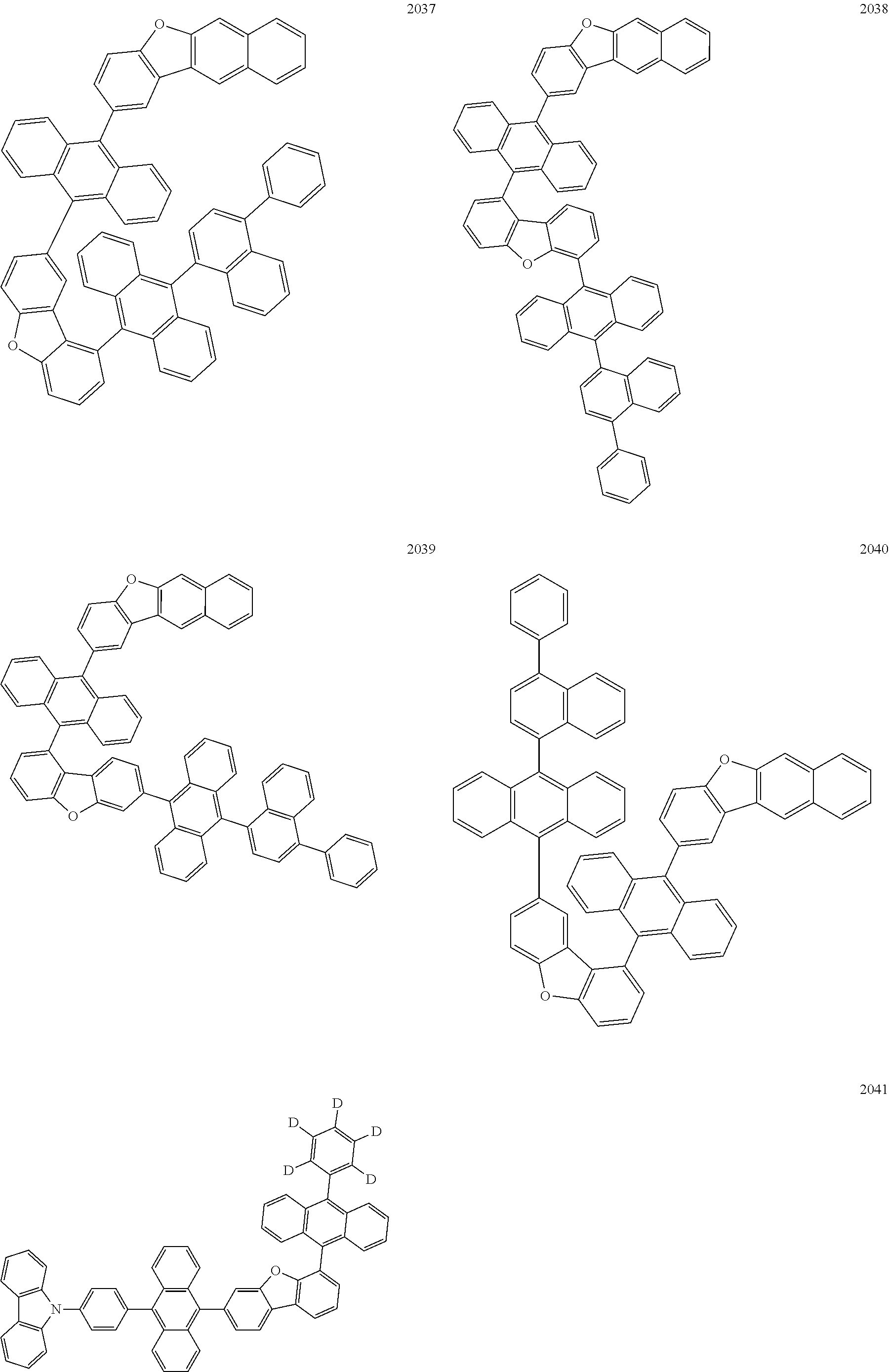

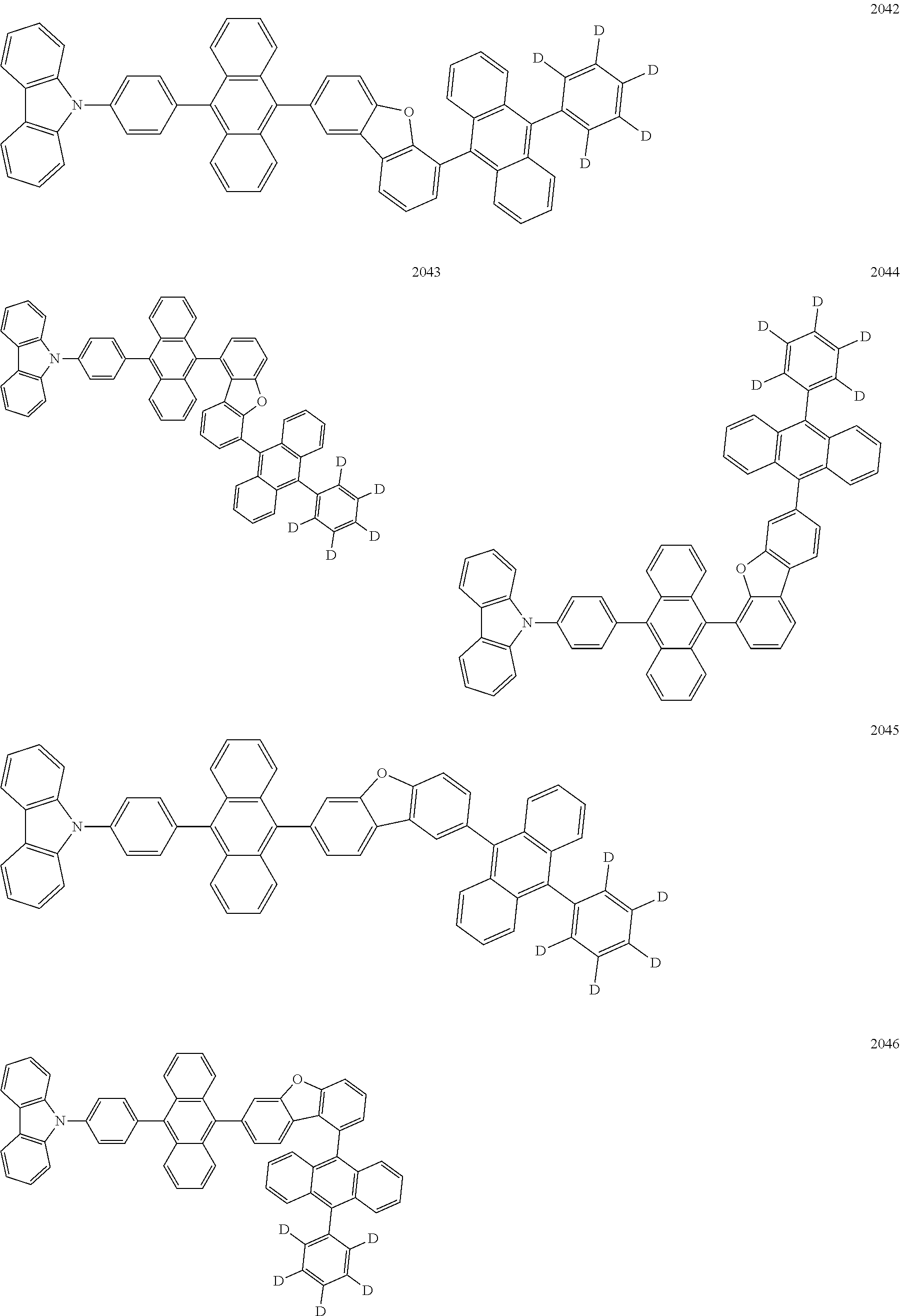

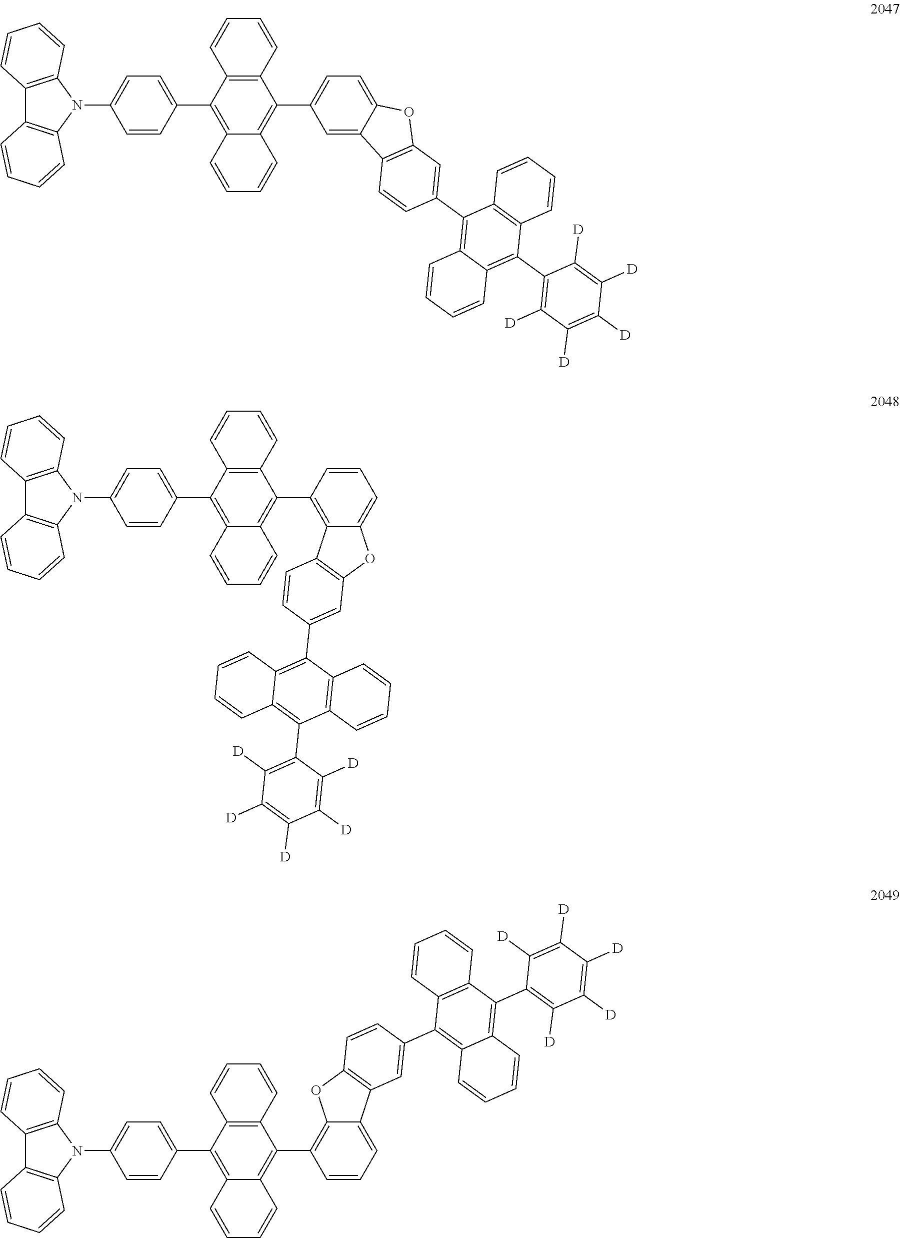 [SMILES: [2H]c1c([2H])c([2H])c(-c2c3ccccc3c(-c3ccc4c(c3)oc3ccc(-c5c6ccccc6c(-c6ccc(-n7c8ccccc8c8ccccc87)cc6)c6ccccc56)cc34)c3ccccc23)c([2H])c1[2H] ; [2H]c1c([2H])c([2H])c(-c2c3ccccc3c(-c3ccc4c(c3)oc3cccc(-c5c6ccccc6c(-c6ccc(-n7c8ccccc8c8ccccc87)cc6)c6ccccc56)c34)c3ccccc23)c([2H])c1[2H] ; [2H]c1c([2H])c([2H])c(-c2c3ccccc3c(-c3ccc4oc5c(-c6c7ccccc7c(-c7ccc(-n8c9ccccc9c9ccccc98)cc7)c7ccccc67)cccc5c4c3)c3ccccc23)c([2H])c1[2H]]

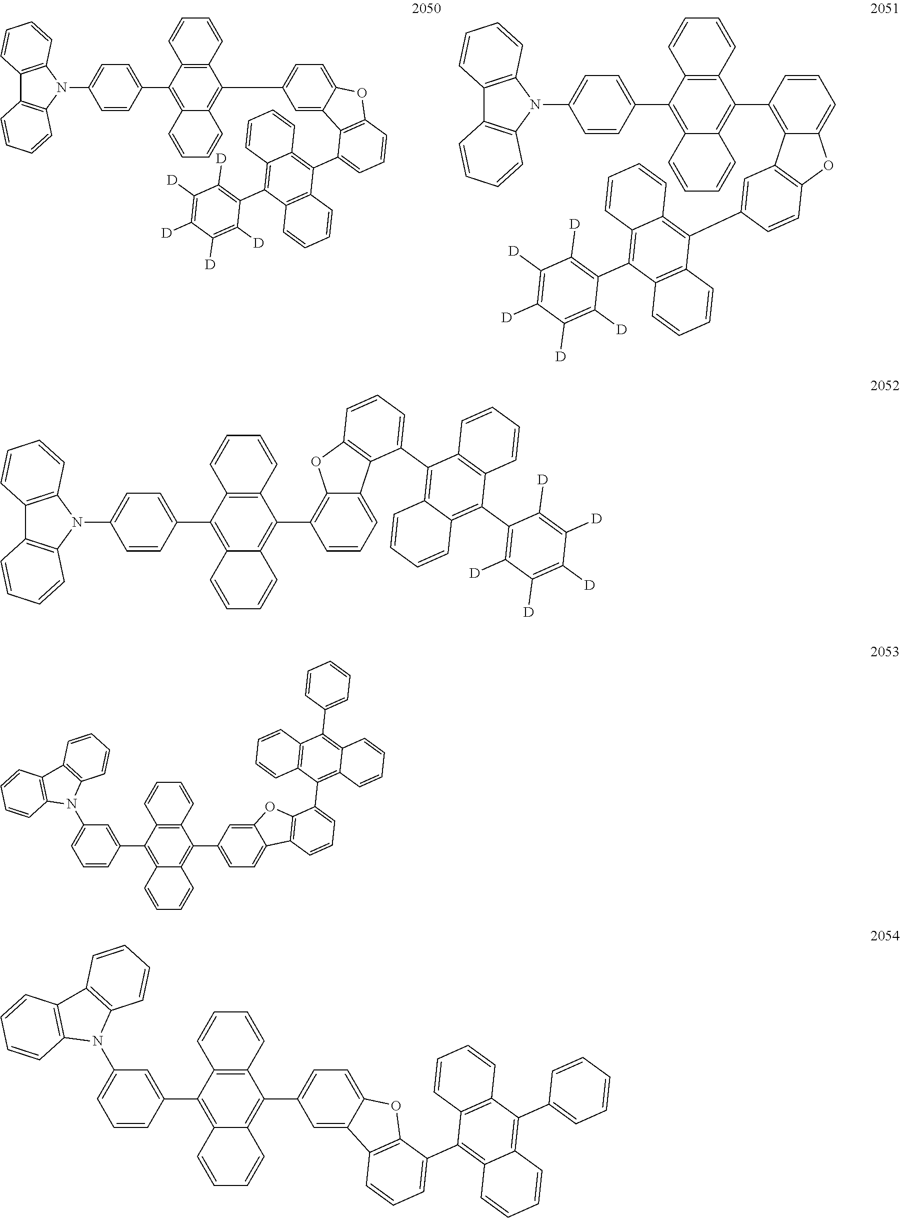

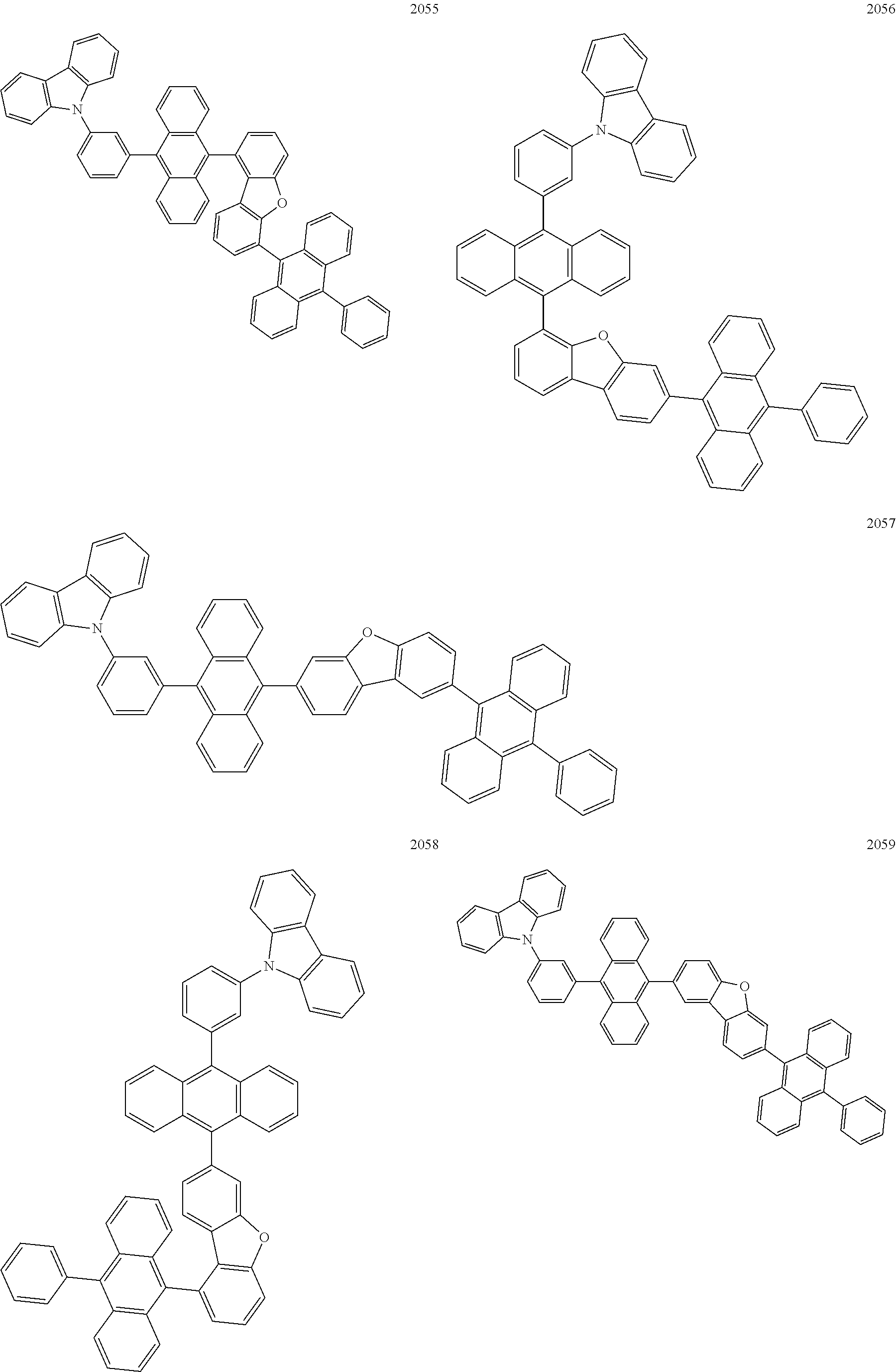

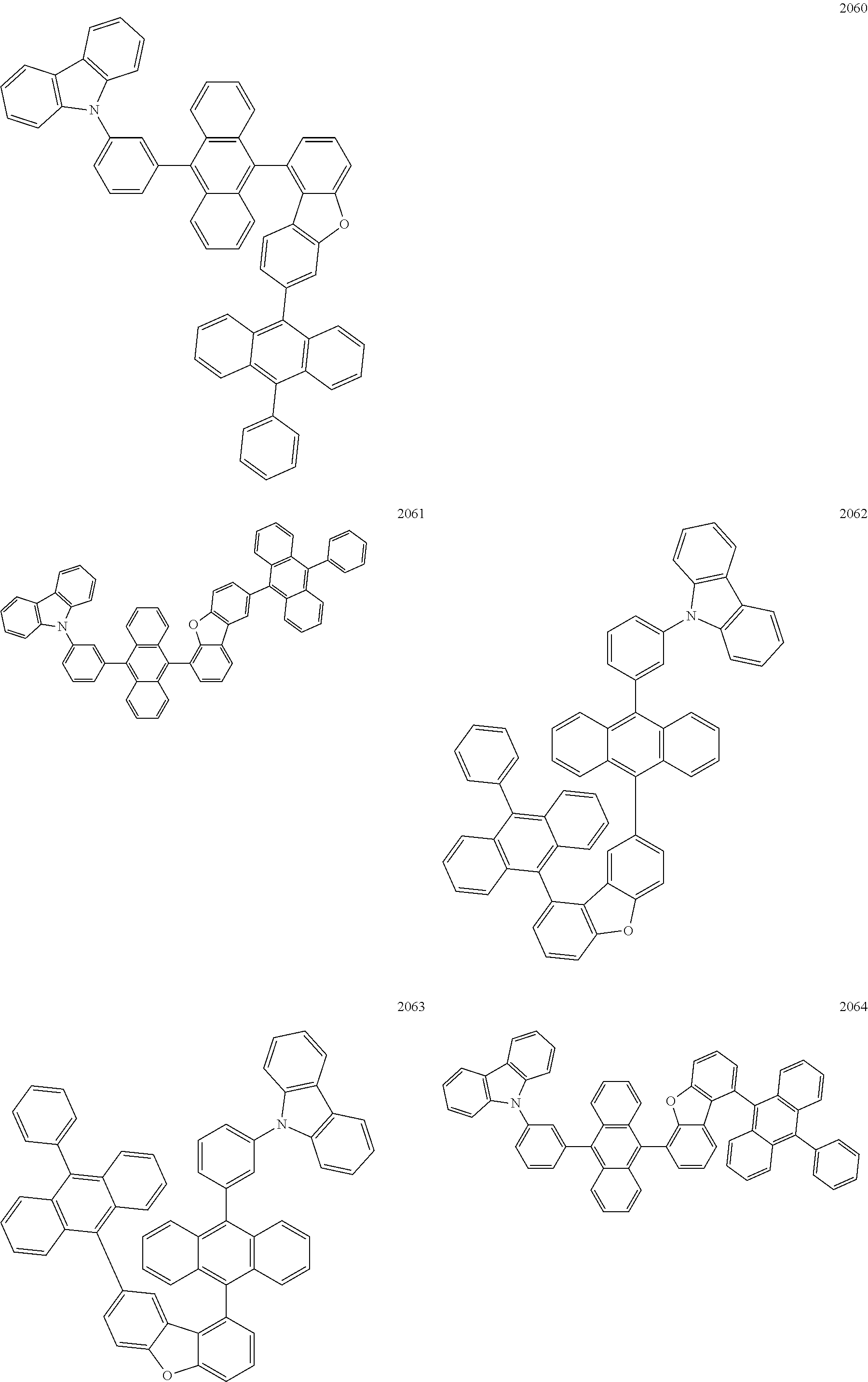

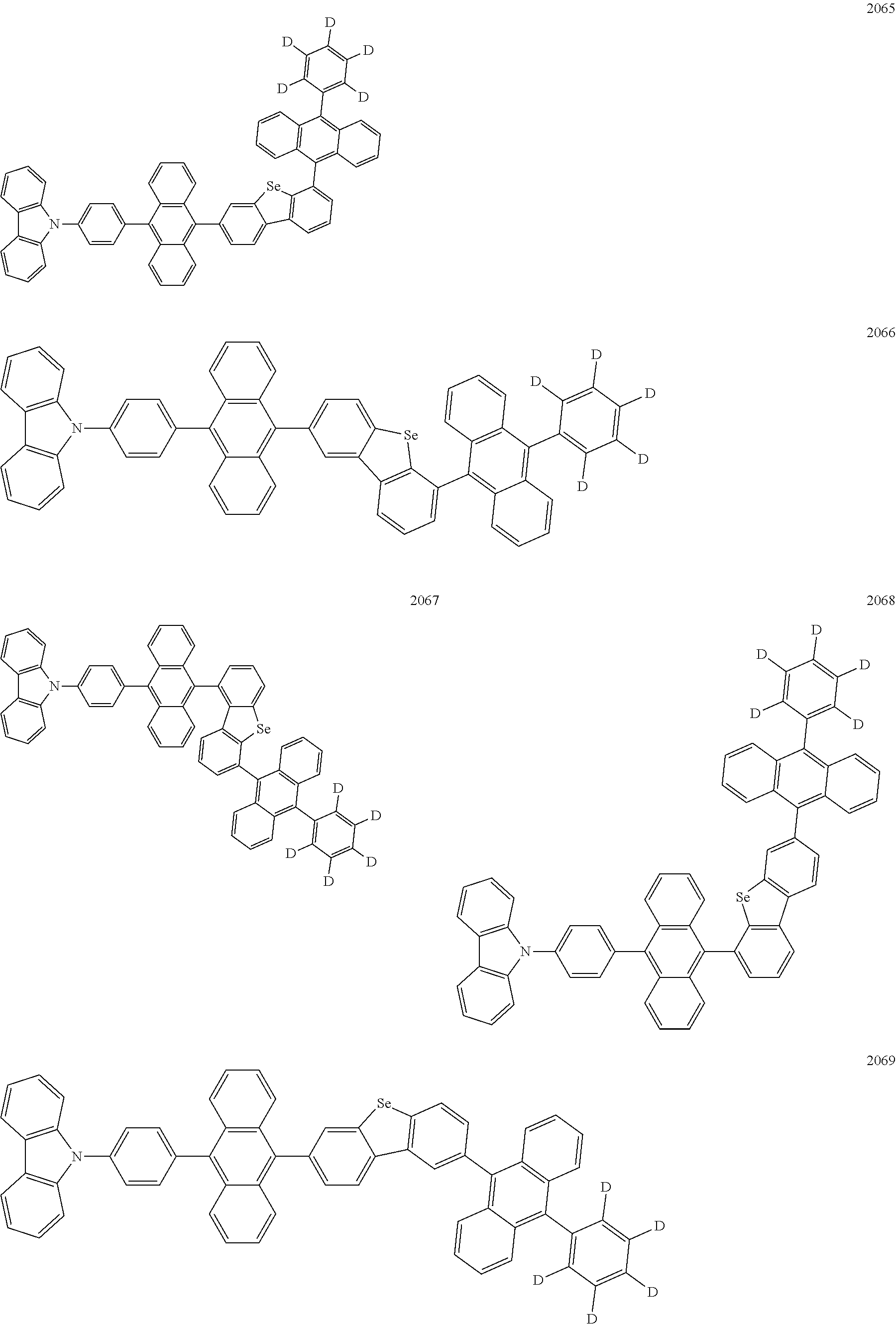

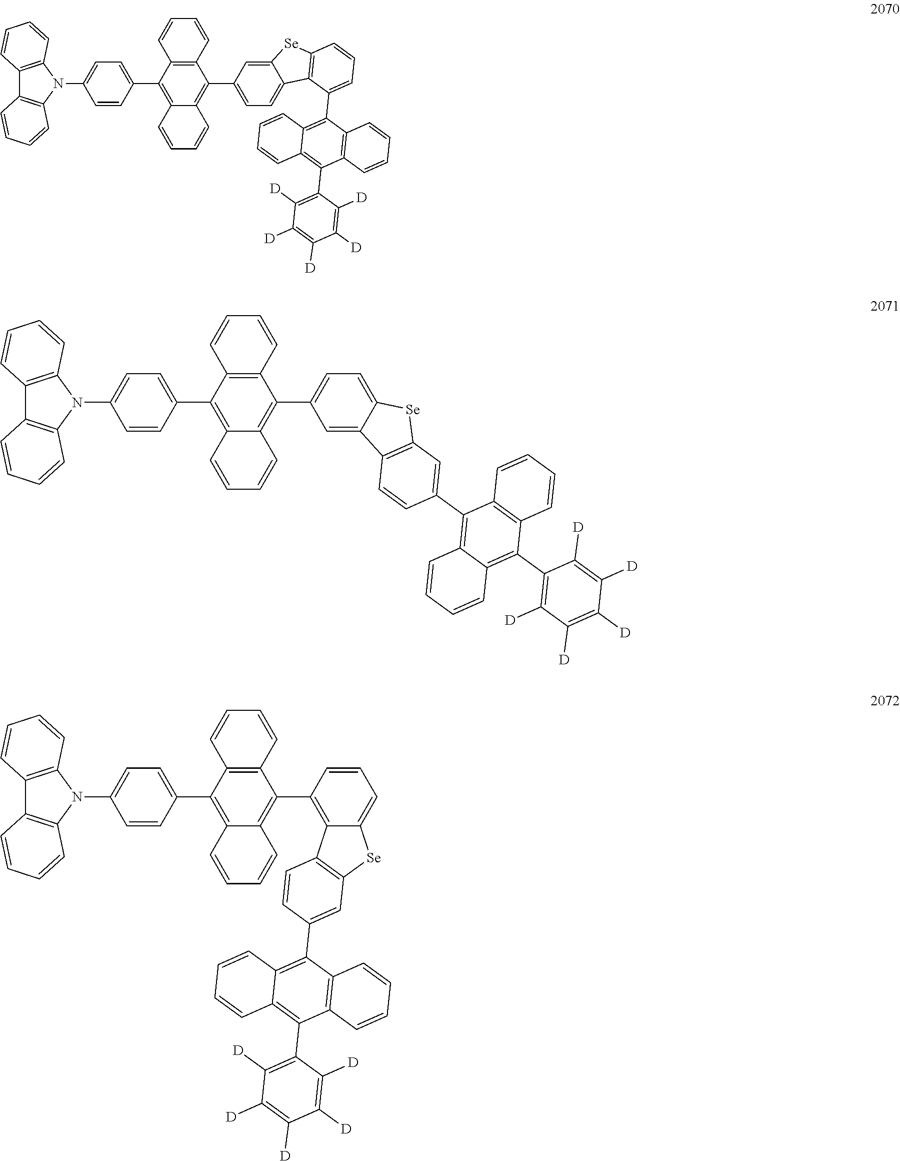 [SMILES: [2H]c1c([2H])c([2H])c(-c2c3ccccc3c(-c3cccc4[se]c5cc(-c6c7ccccc7c(-c7ccc(-n8c9ccccc9c9ccccc98)cc7)c7ccccc67)ccc5c34)c3ccccc23)c([2H])c1[2H] ; [2H]c1c([2H])c([2H])c(-c2c3ccccc3c(-c3ccc4c(c3)[se]c3ccc(-c5c6ccccc6c(-c6ccc(-n7c8ccccc8c8ccccc87)cc6)c6ccccc56)cc34)c3ccccc23)c([2H])c1[2H] ; [2H]c1c([2H])c([2H])c(-c2c3ccccc3c(-c3ccc4c(c3)[se]c3cccc(-c5c6ccccc6c(-c6ccc(-n7c8ccccc8c8ccccc87)cc6)c6ccccc56)c34)c3ccccc23)c([2H])c1[2H]]

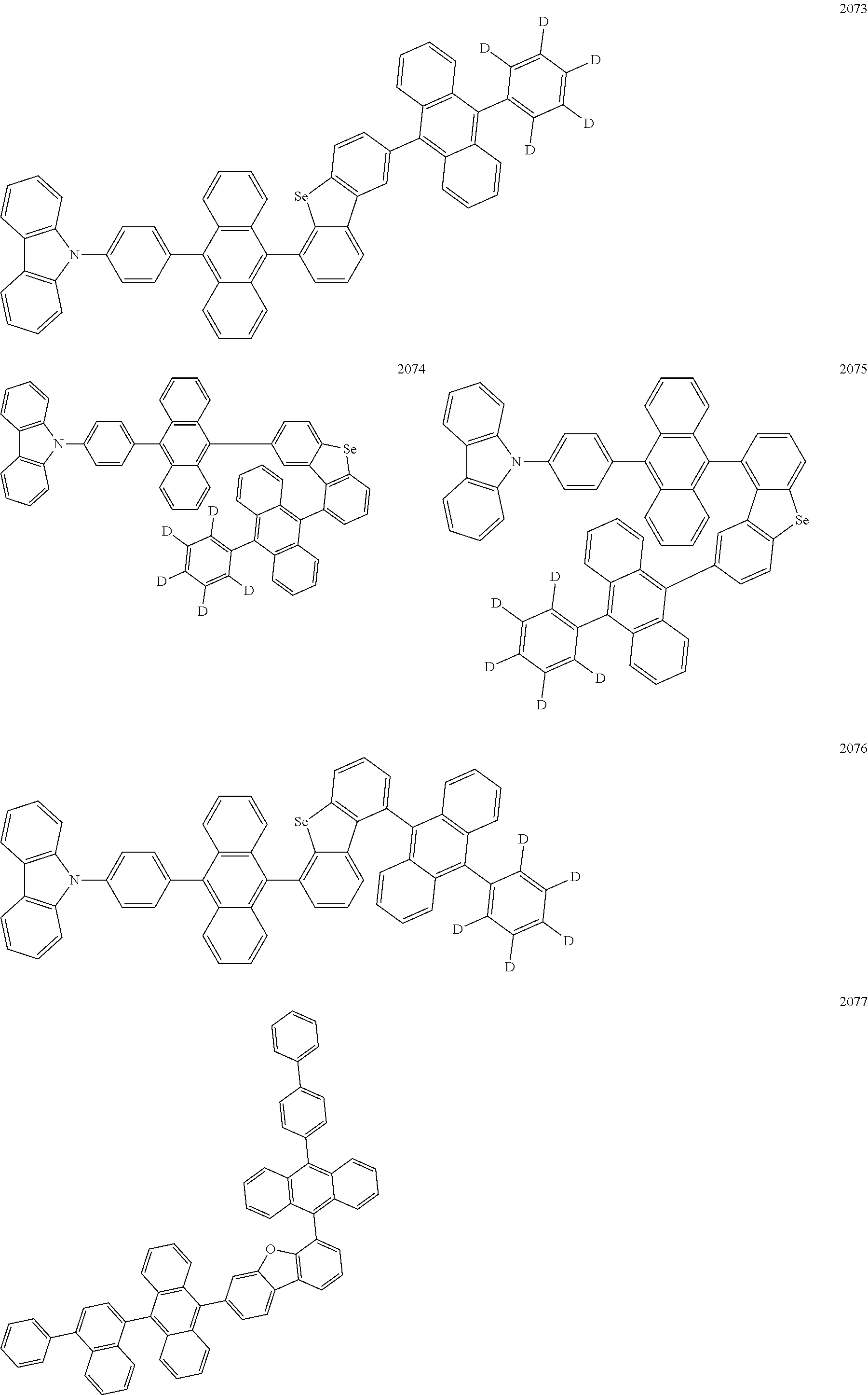

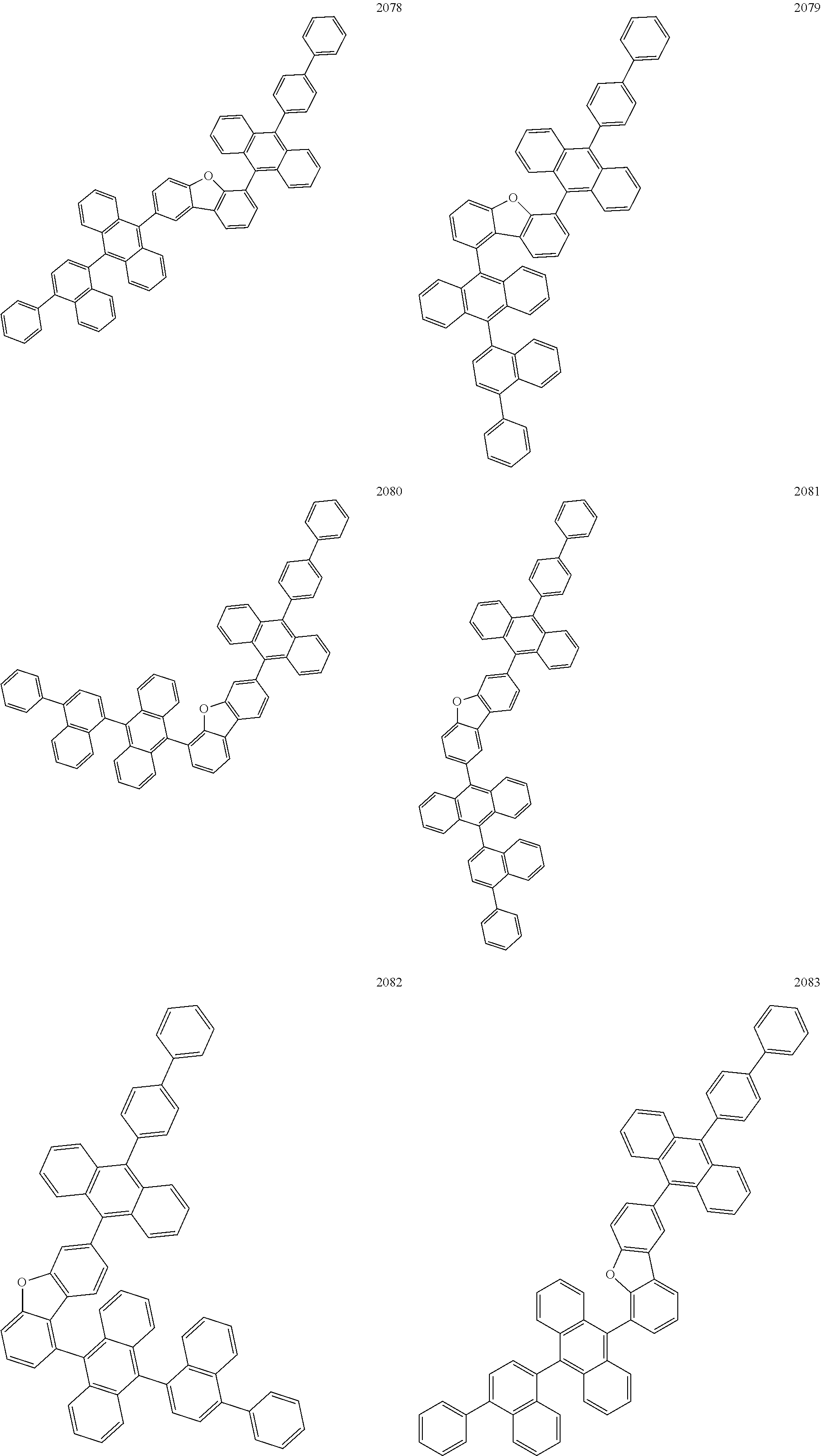

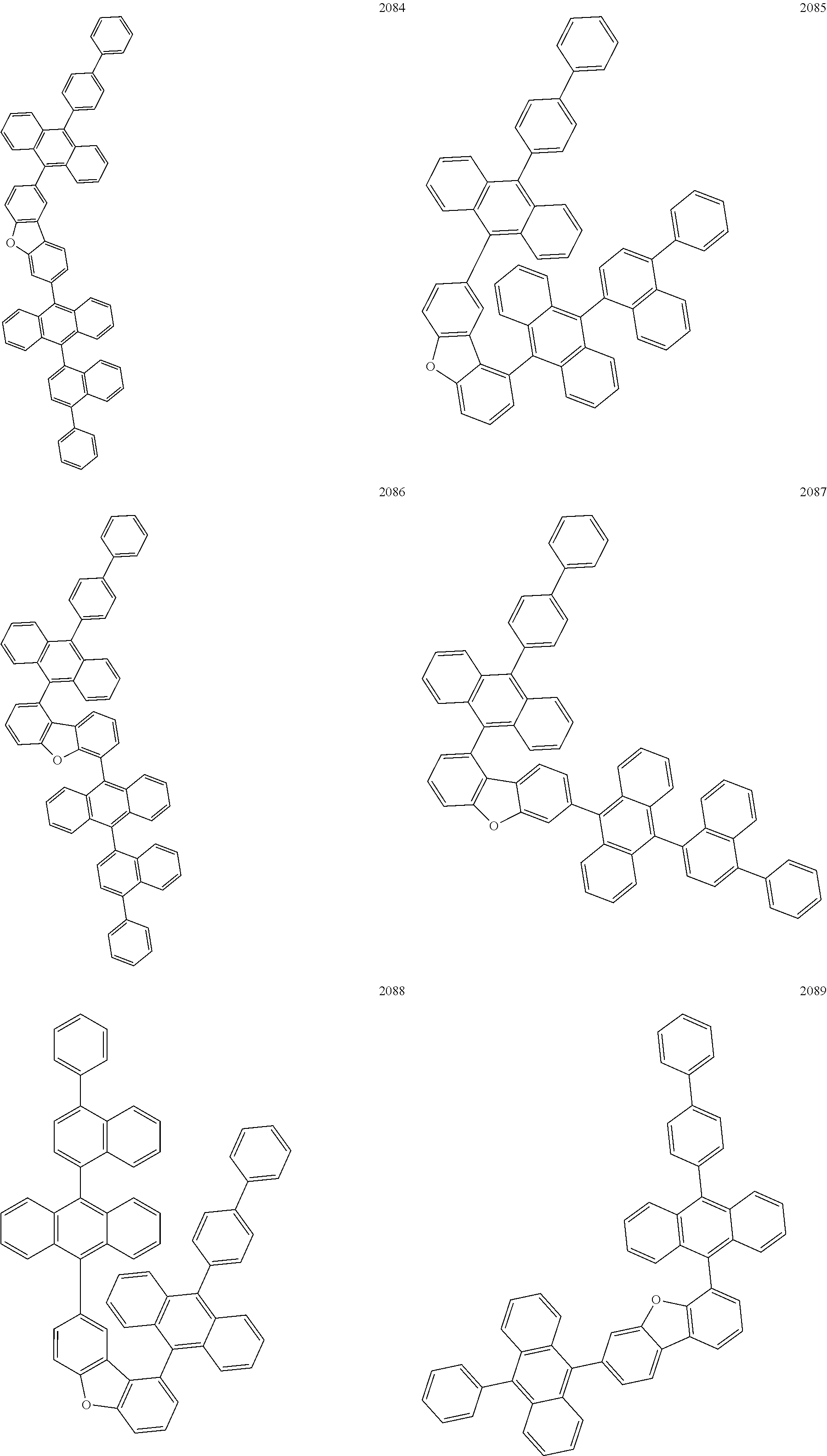

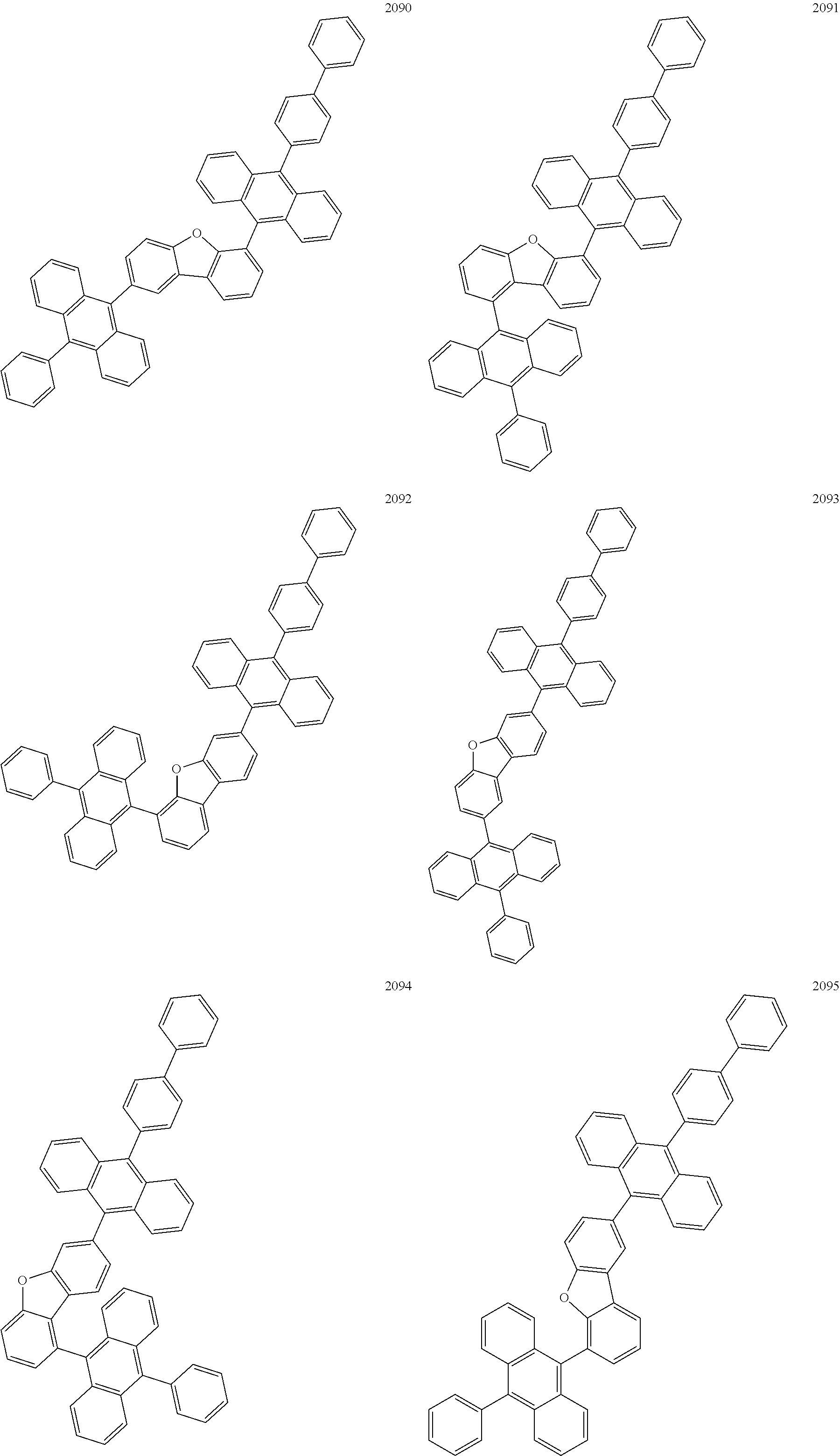

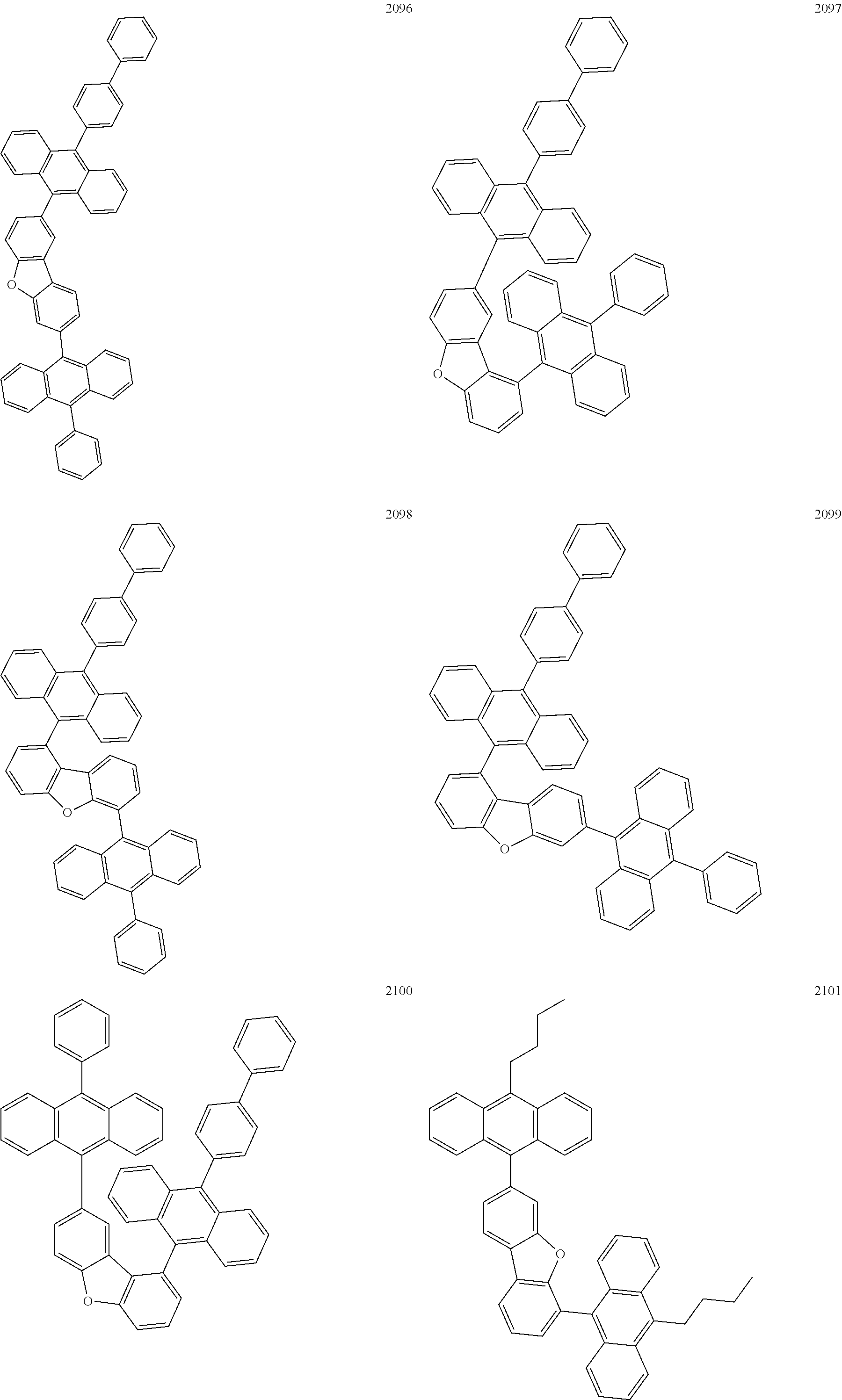

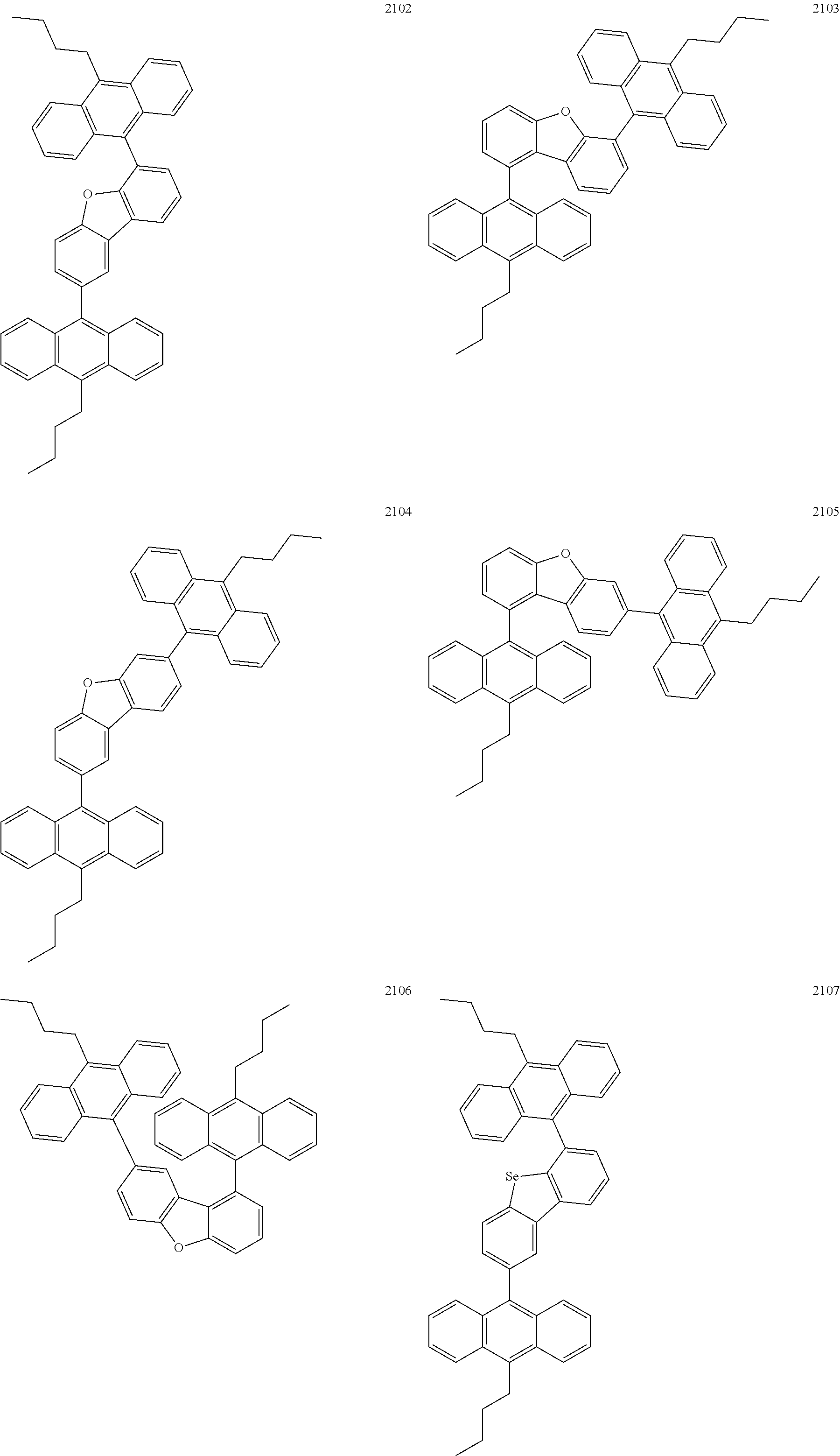

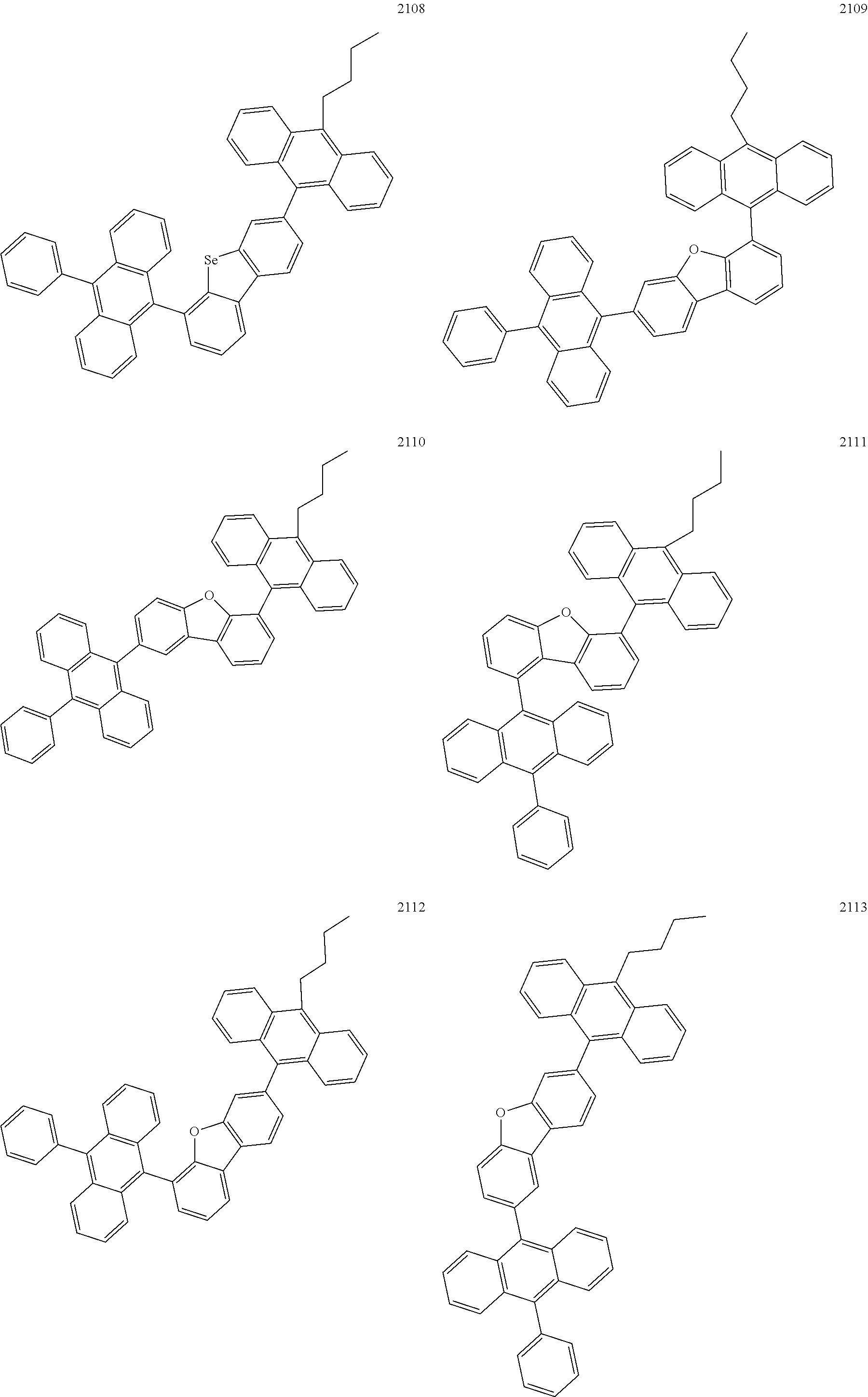

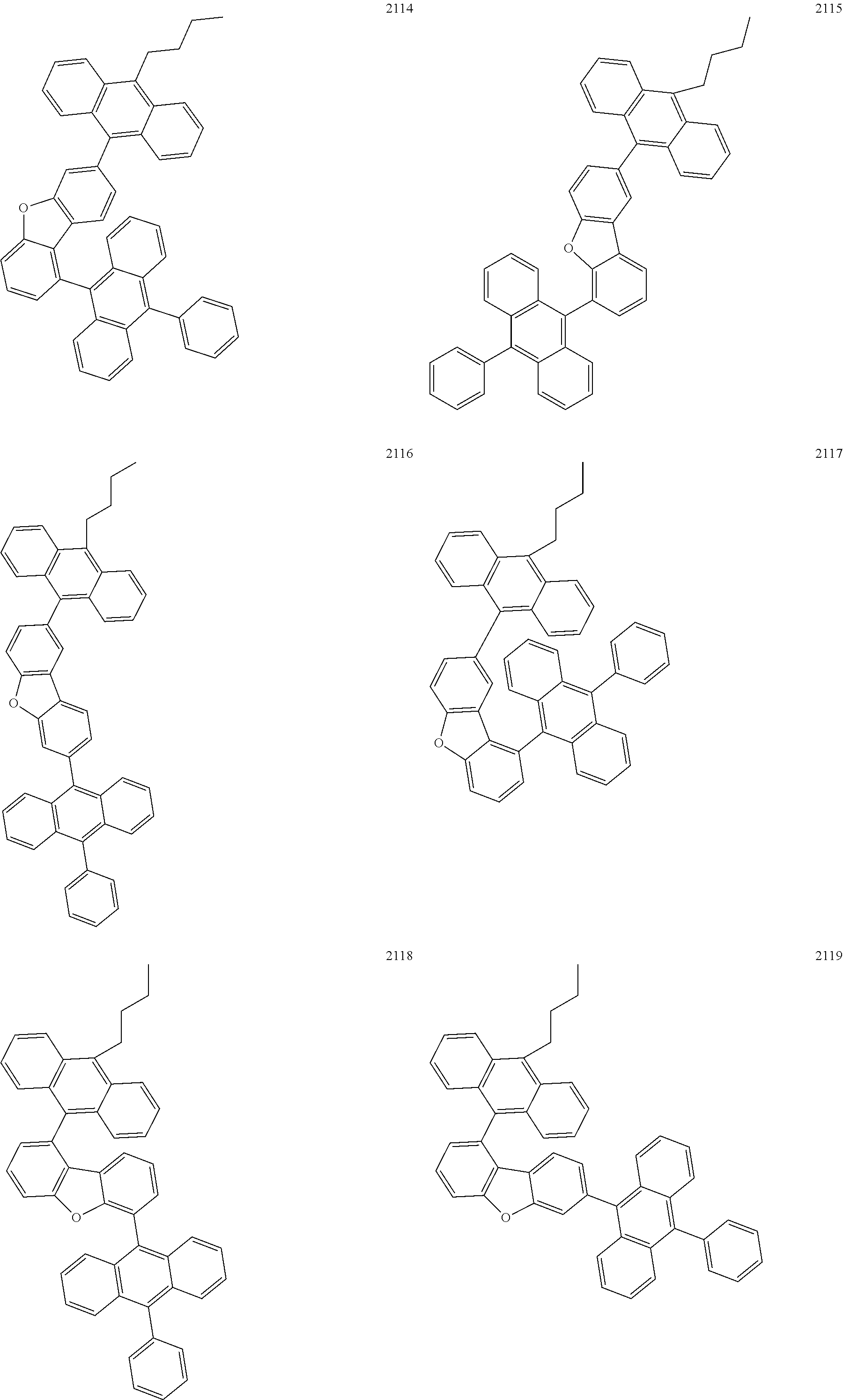 [SMILES: CCCCc1c2ccccc2c(-c2ccc3c(c2)oc2cccc(-c4c5ccccc5c(-c5ccccc5)c5ccccc45)c23)c2ccccc12 ; CCCCc1c2ccccc2c(-c2ccc3oc4c(-c5c6ccccc6c(-c6ccccc6)c6ccccc56)cccc4c3c2)c2ccccc12 ; CCCCc1c2ccccc2c(-c2ccc3oc4cc(-c5c6ccccc6c(-c6ccccc6)c6ccccc56)ccc4c3c2)c2ccccc12 ; CCCCc1c2ccccc2c(-c2ccc3oc4cccc(-c5c6ccccc6c(-c6ccccc6)c6ccccc56)c4c3c2)c2ccccc12 ; CCCCc1c2ccccc2c(-c2cccc3oc4c(-c5c6ccccc6c(-c6ccccc6)c6ccccc56)cccc4c23)c2ccccc12 ; CCCCc1c2ccccc2c(-c2cccc3oc4cc(-c5c6ccccc6c(-c6ccccc6)c6ccccc56)ccc4c23)c2ccccc12]

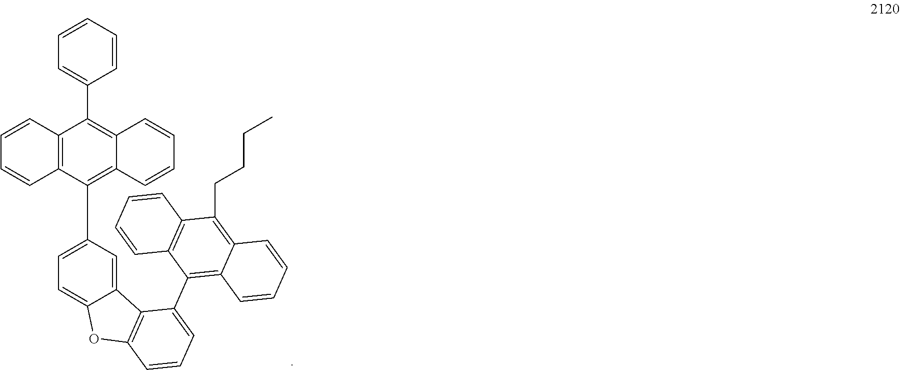 [SMILES: CCCCc1c2ccccc2c(-c2cccc3oc4ccc(-c5c6ccccc6c(-c6ccccc6)c6ccccc56)cc4c23)c2ccccc12]

When the host is a mixture of an electron transporting host and a hole transporting host, a weight ratio of the electron transporting host to the hole transporting host may be in a range of about 1:9 to about 9:1, for example, about 2:8 to about 8:2, for example about 4:6 to about 6:4, or for example, about 5:5. When a weight ratio of the electron transporting host to a hole transporting host is within any of these ranges, holes and electrons transport balance into the emission layer 15 may be achieved.

Dopant in emission layer 15

The dopant may include the heterocyclic compound.

Sensitizer in emission layer 15

In some embodiments, the sensitizer may be a phosphorescence sensitizer including at least one metal a first-row transition metal, a second-row transition metal, a third-row transition metal, or any combination thereof.

In some embodiments, the sensitizer may include a metal (M₁₁) which may include at least one metal of a first-row transition metal, a second-row transition metal, a third-row transition metal, or any combination thereof, and an organic ligand (L₁₁), and L₁₁ and M₁₁ may form 1, 2, 3, or 4 cyclometallated ring.

In some embodiments, the sensitizer may include an organometallic compound represented by Formula 101:

M₁₁(L₁₁)ₙ₁₁(L₁₂)ₙ₁₂ Formula 101

wherein, in Formula 101,

M₁₁ may be a first-row transition metal, a second-row transition metal, or a third-row transition metal,

L₁₁ may be a ligand represented by one of Formulae 1-1 to 1-4,

L₁₂ may be a monodentate ligand or a bidentate ligand,

n11 may be 1, and

n12 may be 0, 1, or 2:

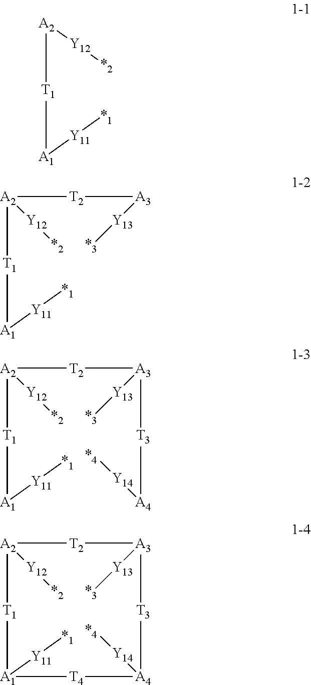

wherein, in Formulae 1-1 to 1-4,

A₁ to A₄ may each independently be a substituted or unsubstituted C₅-C₃₀ carbocyclic group, a substituted or unsubstituted C₁-C₃₀ heterocyclic group, or a non-cyclic group,

Y₁₁ to Y₁₄ may each independently be a chemical bond, O, S, N(R₉₁), B(R₉₁), P(R₉₁), or C(R₉₁)(R₉₂),

T₁ to T₄ may each independently be a single bond, a double bond, *—N(R₉₃)—*′, *—B(R₉₃)—*′, *—P(R₉₃)—*′, *—C(R₉₃)(R₉₄)—*′, *—Si(R₉₃)(R₉₄)—*′, *—Ge(R₉₃)(R₉₄)—*′, *S—*′, *—Se—*′, * 0—*′, *—C(═O)—*′, *—S(═O)—*′, *—S(═O)₂—*′, *—C(R₉₃)=*′, *═C(R₉₃)—*′, *—C(R₉₃)═C(R₉₄)—*′, *—C(=S)—*′, or *—C—C—*′,

a substituent of the substituted C₅-C₃₀ carbocyclic group, a substituent of the substituted C₁-C₃₀ heterocyclic group, and R₉₁ to R₉₄ may each independently be hydrogen, deuterium, —F, —Cl, —Br, —I, —SF₅, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent aromatic condensed polycyclic group, a substituted or unsubstituted monovalent aromatic condensed heteropolycyclic group, a substituted or unsubstituted monovalent non-aromatic condensed polycyclic group, a substituted or unsubstituted monovalent non-aromatic condensed heteropolycyclic group, —Si(Q₁)(Q₂)(Q₃), —Ge(Q₁)(Q₂)(Q₃), —C(Q₁)(Q₂)(Q₃), —B(Q₁)(Q₂), —N(Q₁)(Q₂), —P(Q₁)(Q₂), —C(═O)(Q₁), —S(═O)(Q₁), —S(═O)₂(Q₁), —P(═O)(Q₁)(Q₂), or —P(═S)(Q₁)(Q₂), provided that the substituent of the substituted C₅-C₃₀ carbocyclic group and the substituent of the substituted C₁-C₃₀ heterocyclic group may not each be hydrogen, and

*1, *2, *3, and *4 each indicate a binding site to M₁₁,

wherein Q₁ to Q₃ may each independently be hydrogen, deuterium, —F, —Cl, —Br, —I, a hydroxyl group, a cyano group, a nitro group, an amidino group, a hydrazino group, a hydrazono group, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₇-C₆₀ alkyl aryl group, a C₆-C₆₀ aryloxy group, a C₆-C₆₀ arylthio group, a C₁-C₆₀ heteroaryl group, a C₂-C₆₀ alkyl heteroaryl group, a C₁-C₆₀ heteroaryloxy group, a C₁-C₆₀ heteroarylthio group, a monovalent aromatic condensed polycyclic group, a monovalent aromatic condensed heteropolycyclic group, a monovalent non-aromatic condensed polycyclic group, a monovalent non-aromatic condensed heteropolycyclic group, a C₁-C₆₀ alkyl group substituted with at least one deuterium, —F, a cyano group, a C₁-C₆₀ alkyl group, a C₆-C₆₀ aryl group, or any combination thereof, or a C₆-C₆₀ aryl group substituted with at least one deuterium, —F, a cyano group, a C₁-C₆₀ alkyl group, a C₆-C₆₀ aryl group, or any combination thereof.

In some embodiments, the sensitizer may be of Groups I to VIII, but embodiments are not limited thereto:

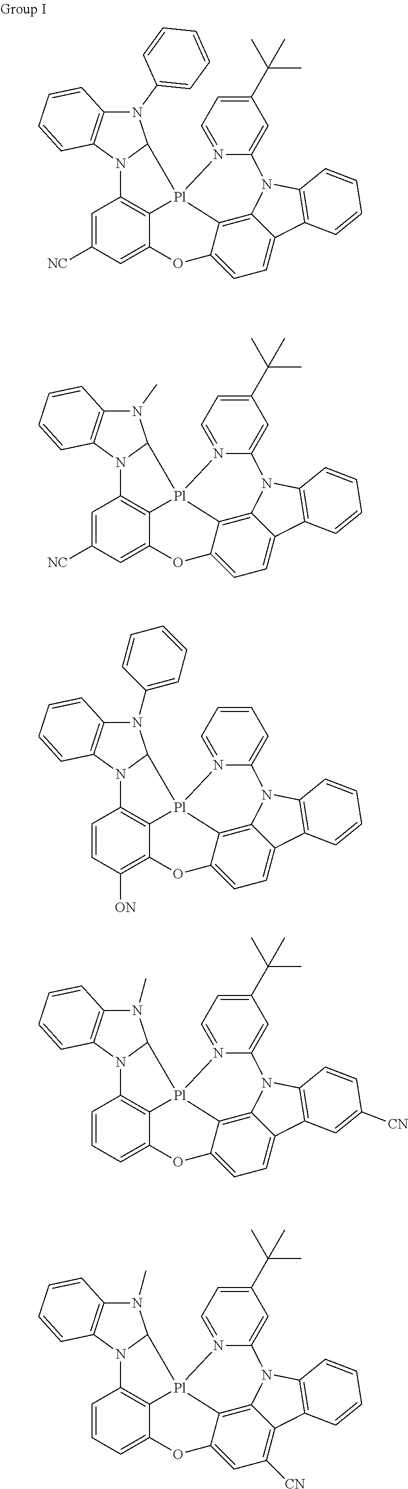

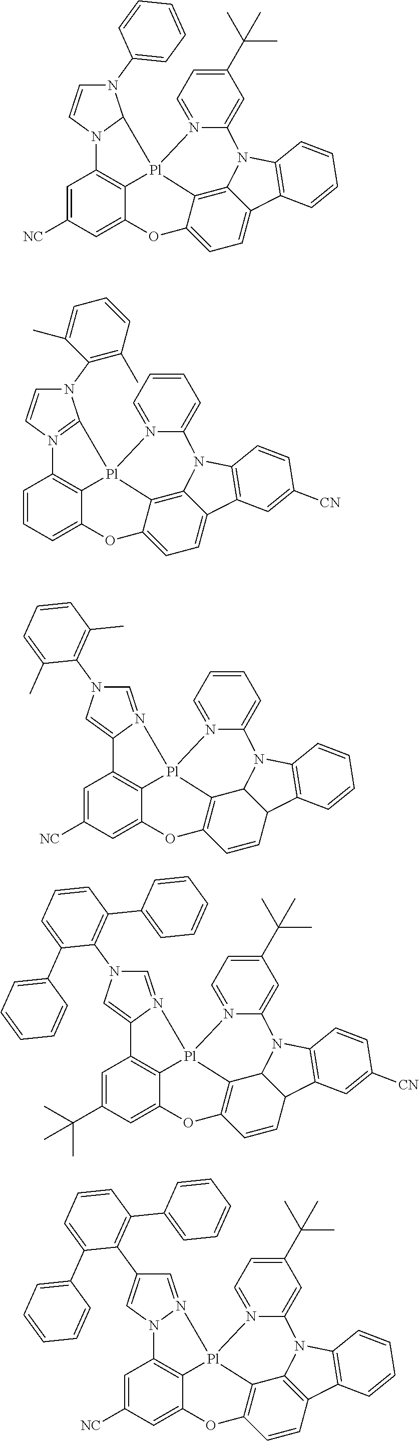

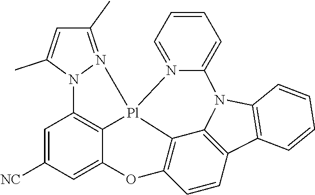

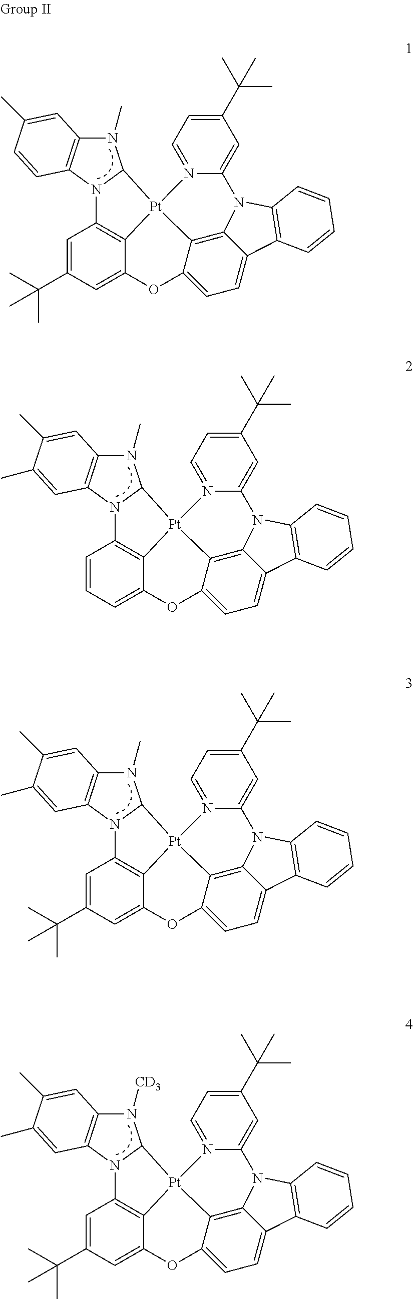

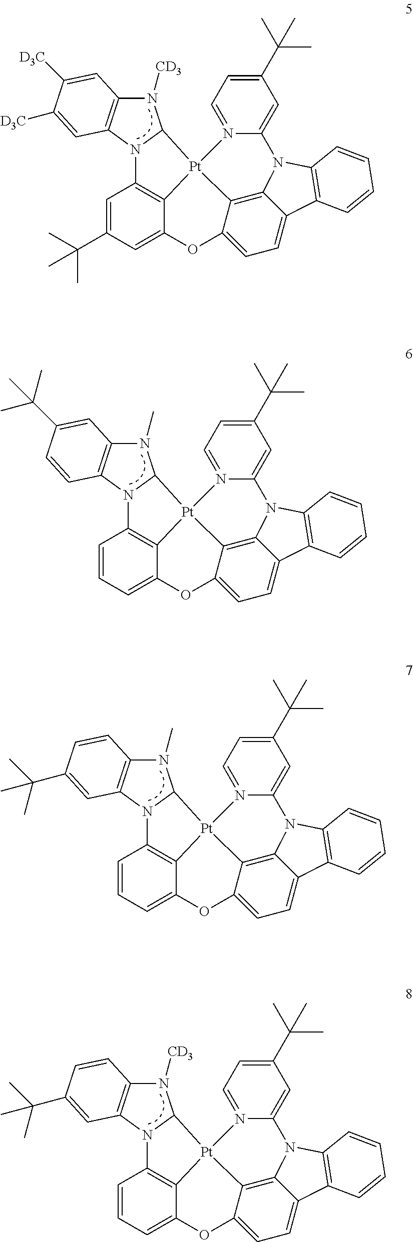

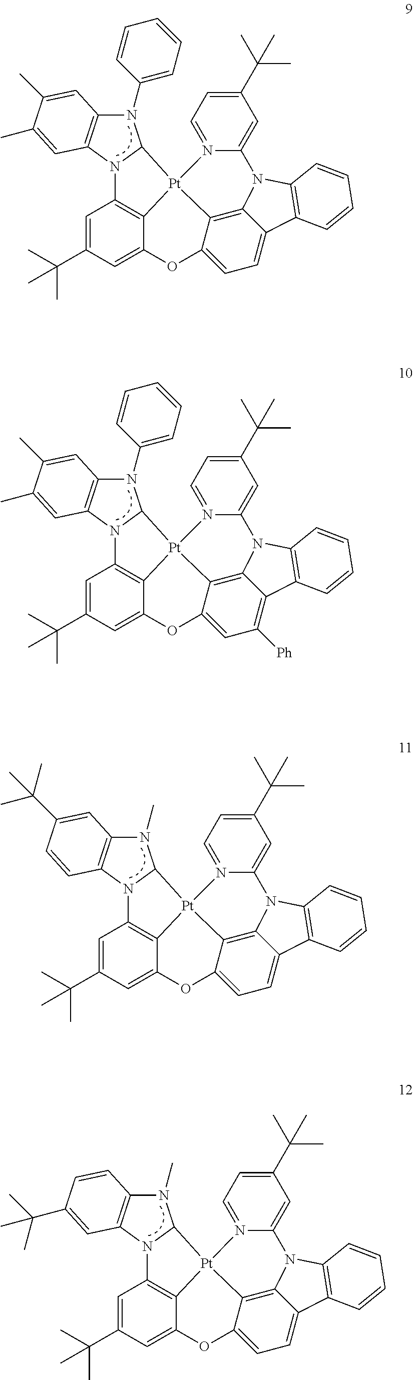

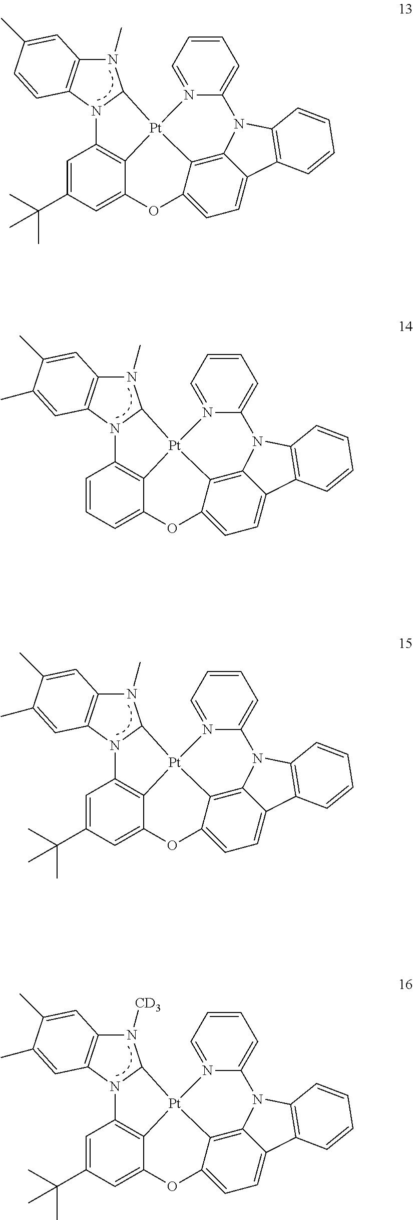

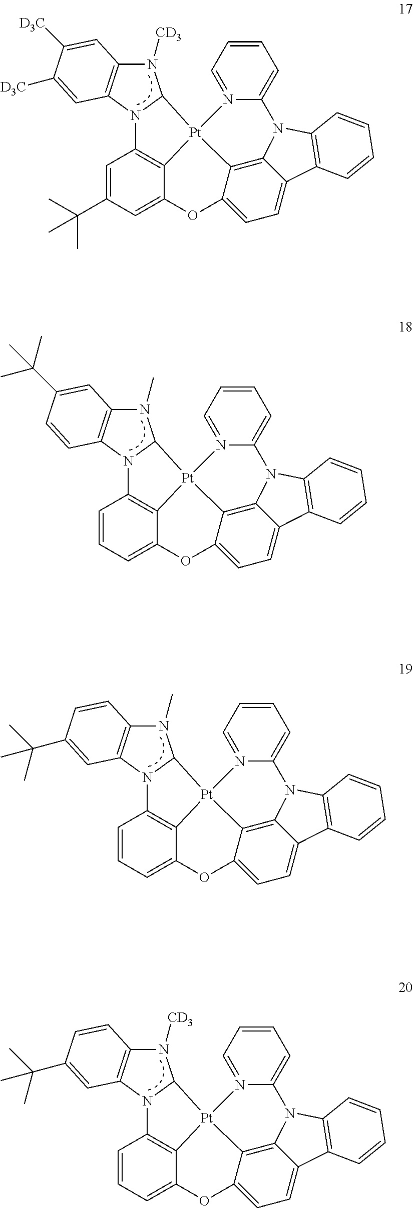

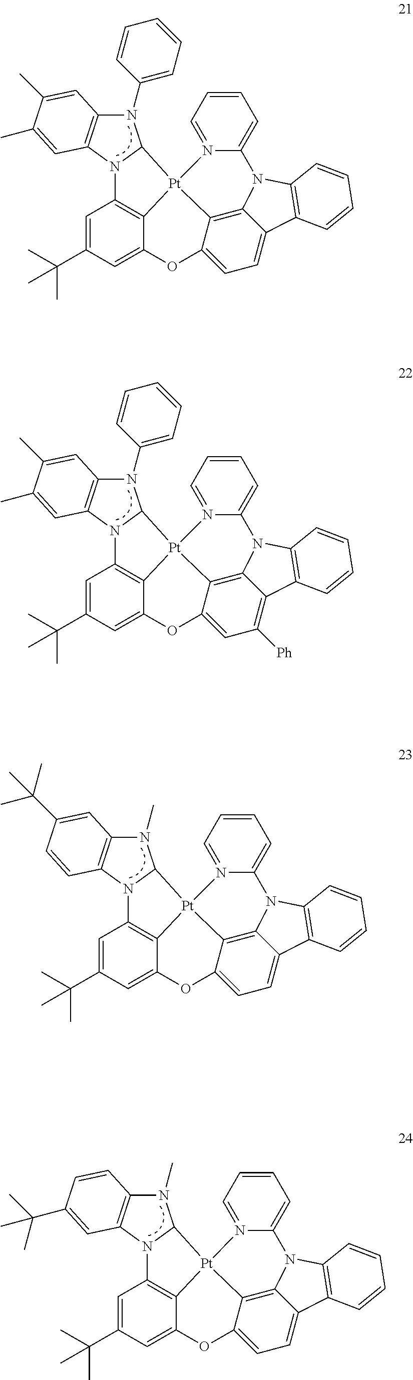

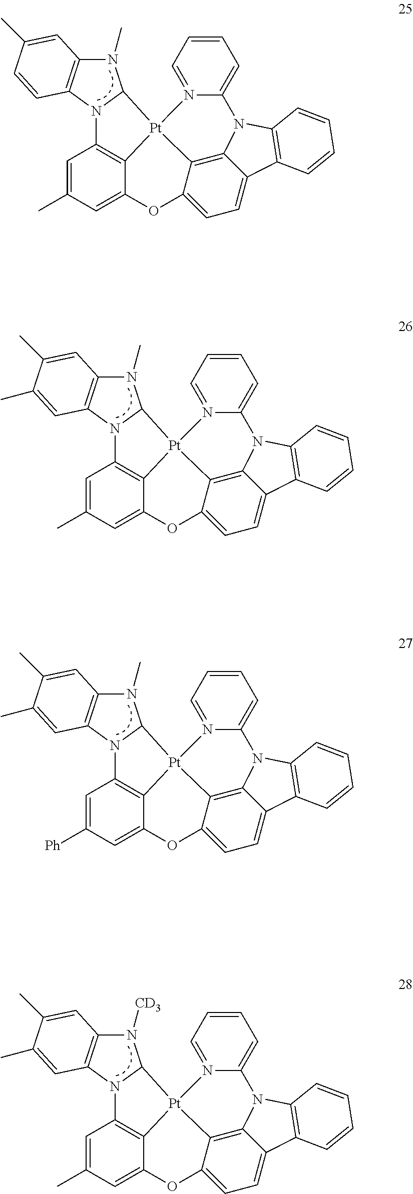

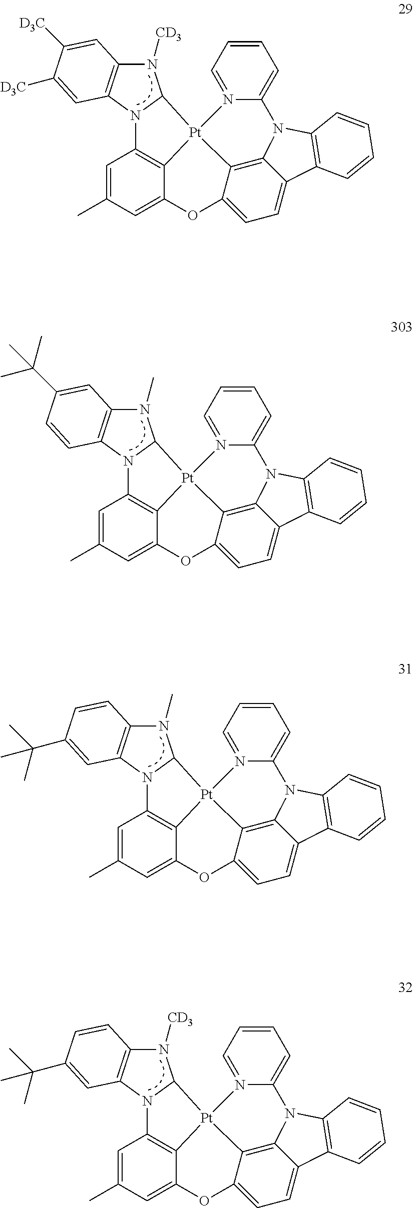

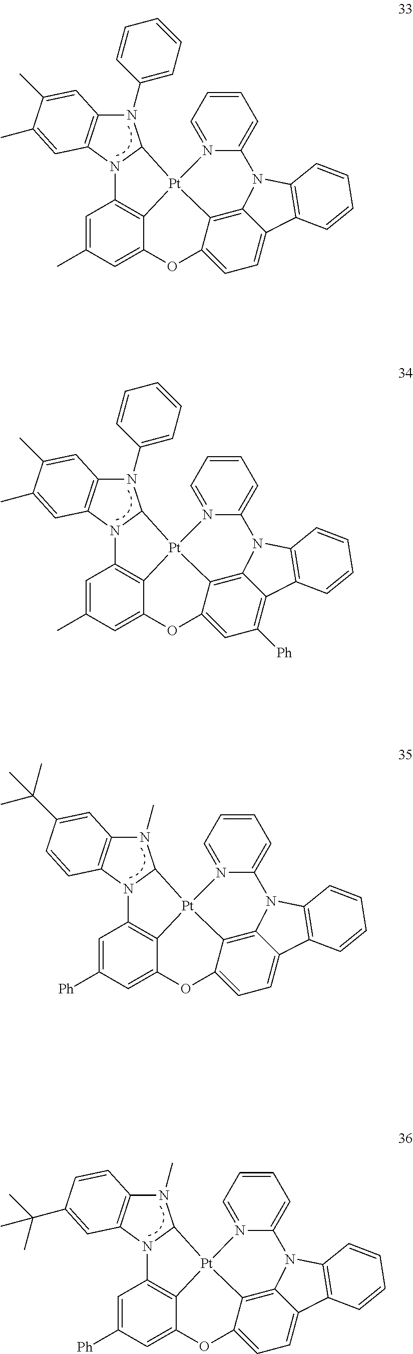

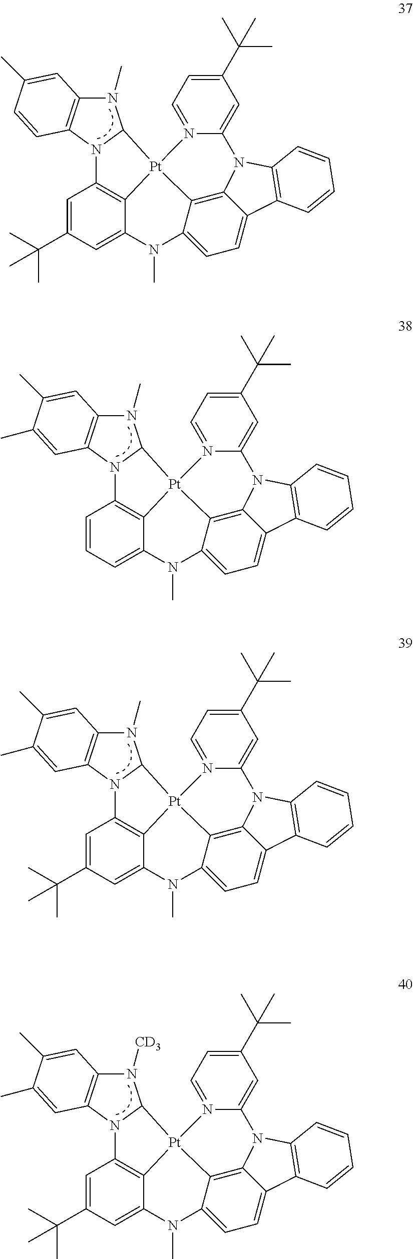

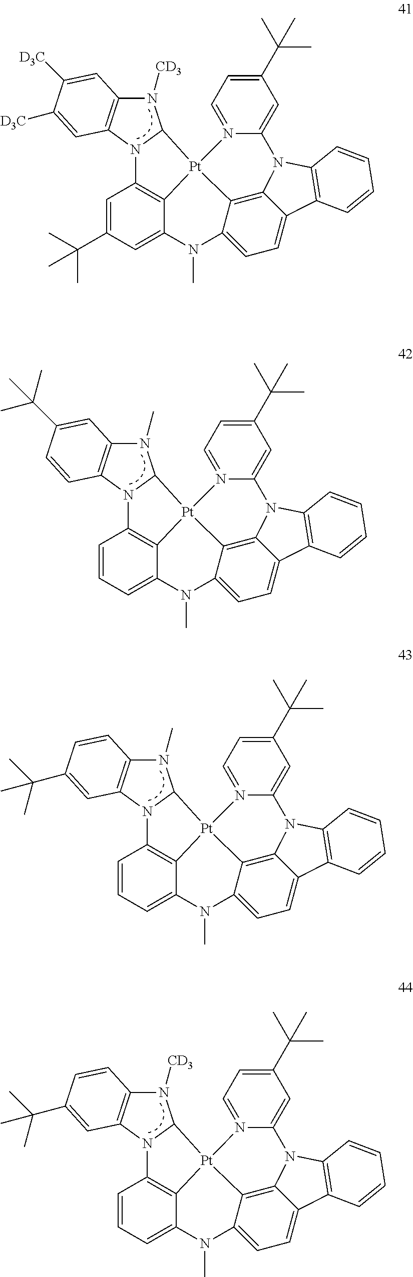

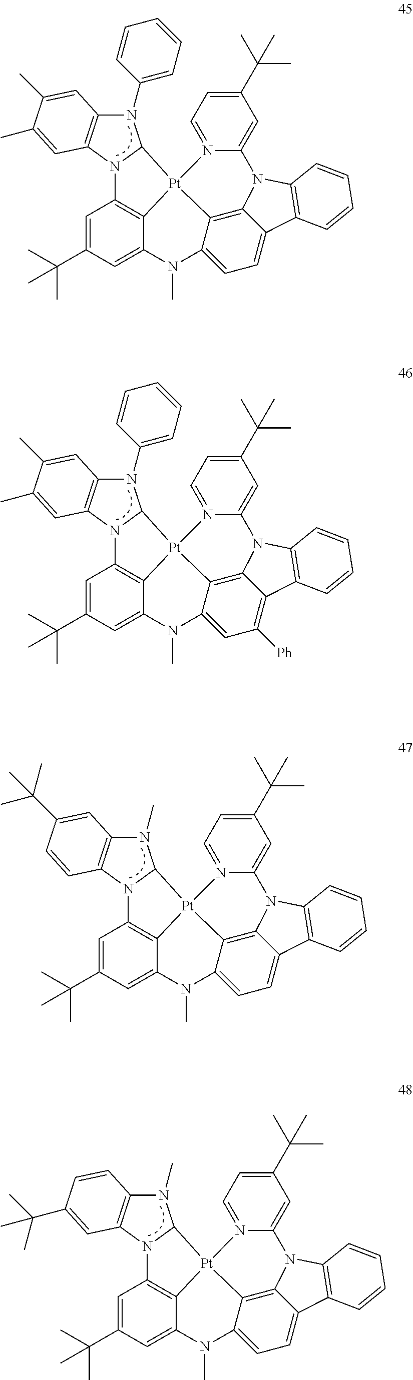

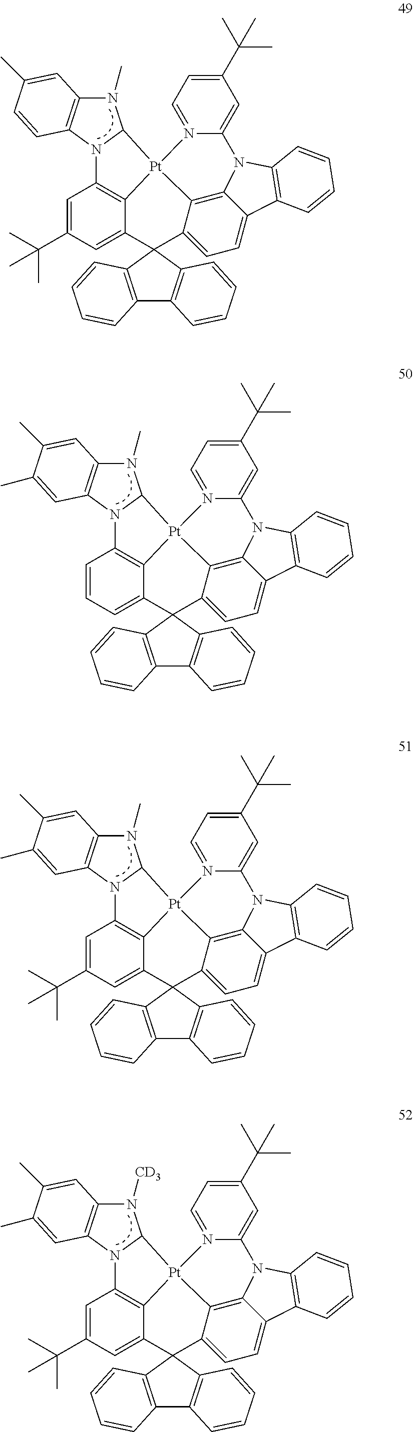

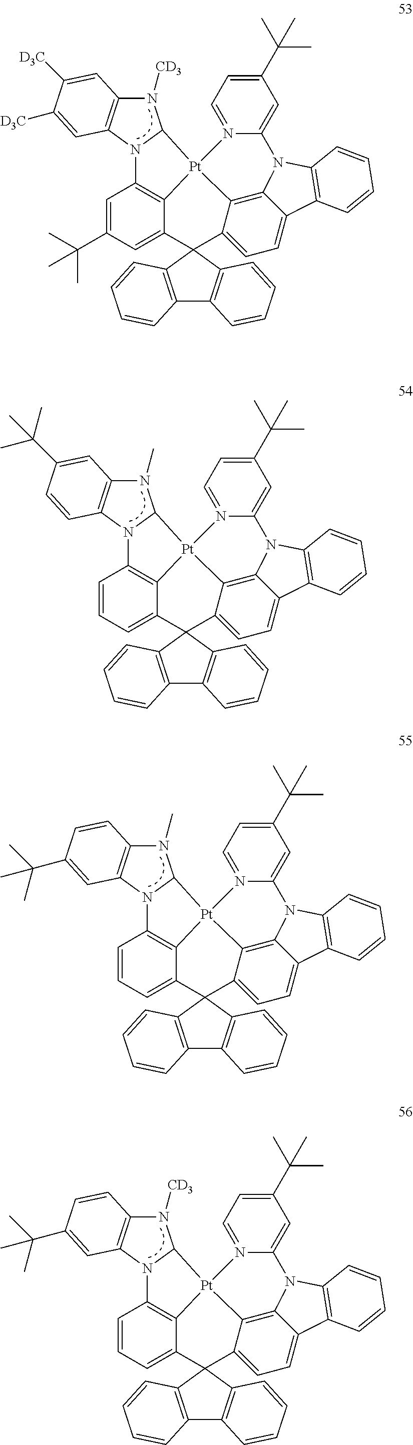

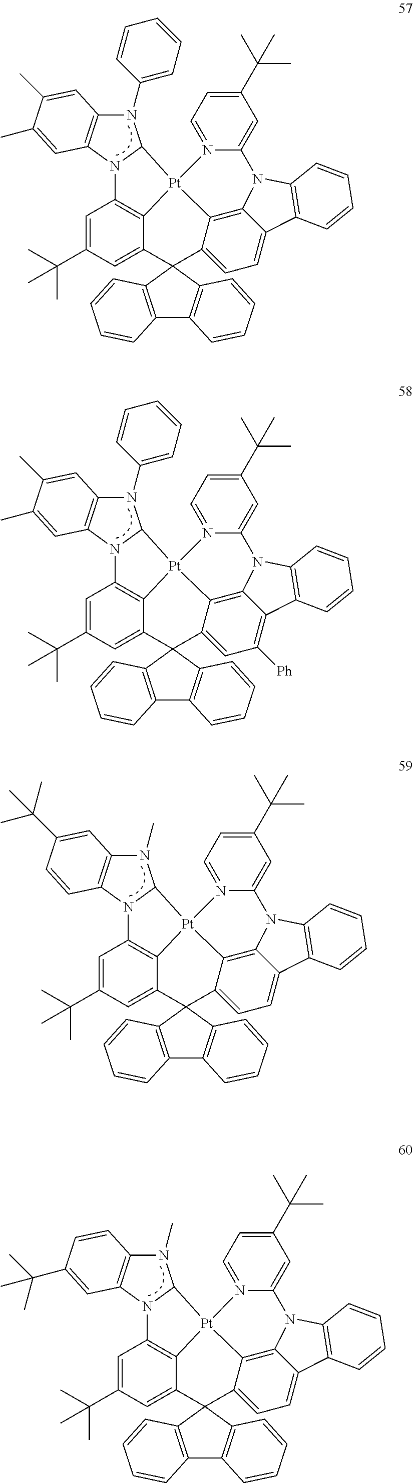

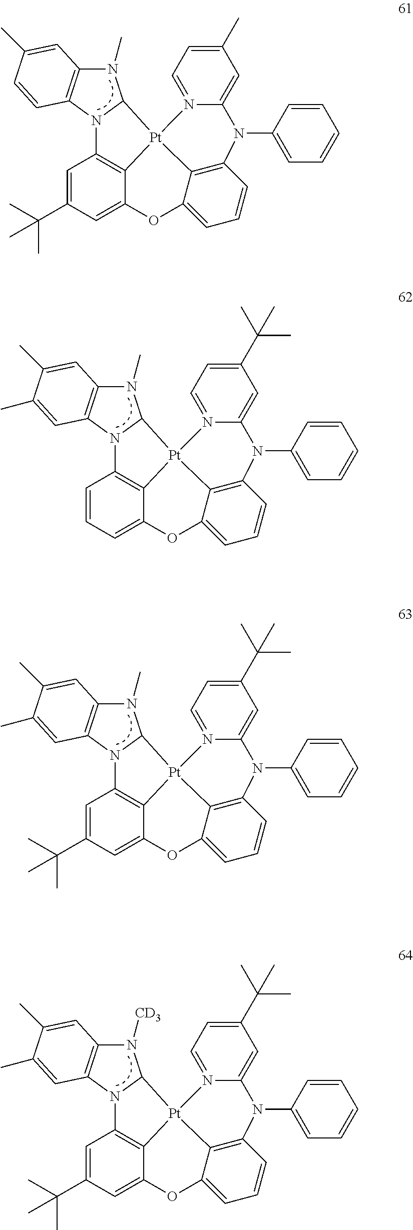

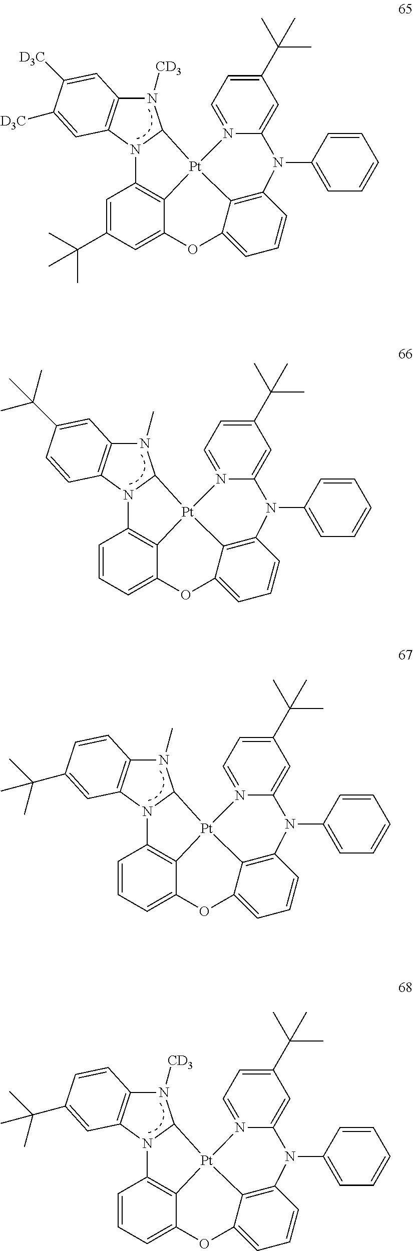

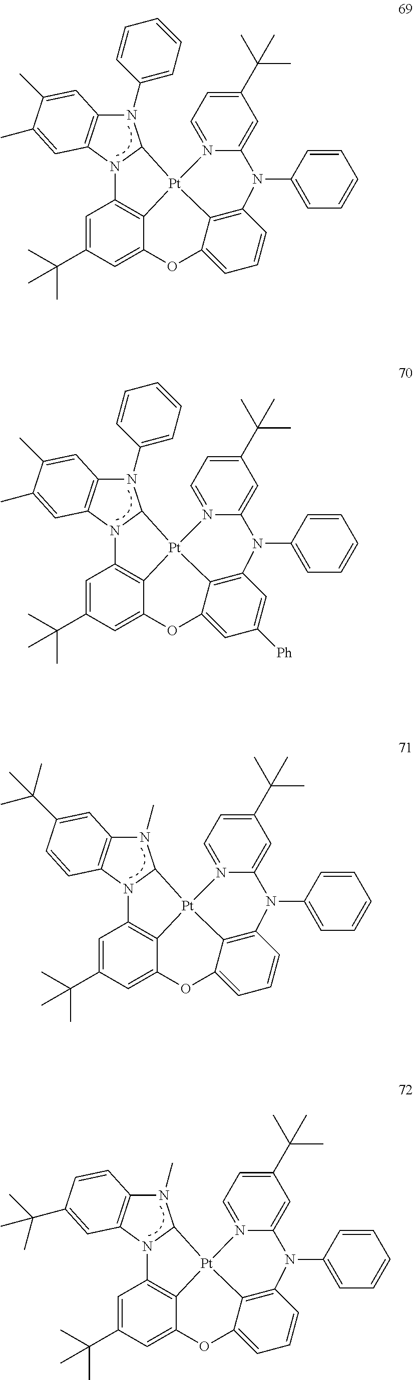

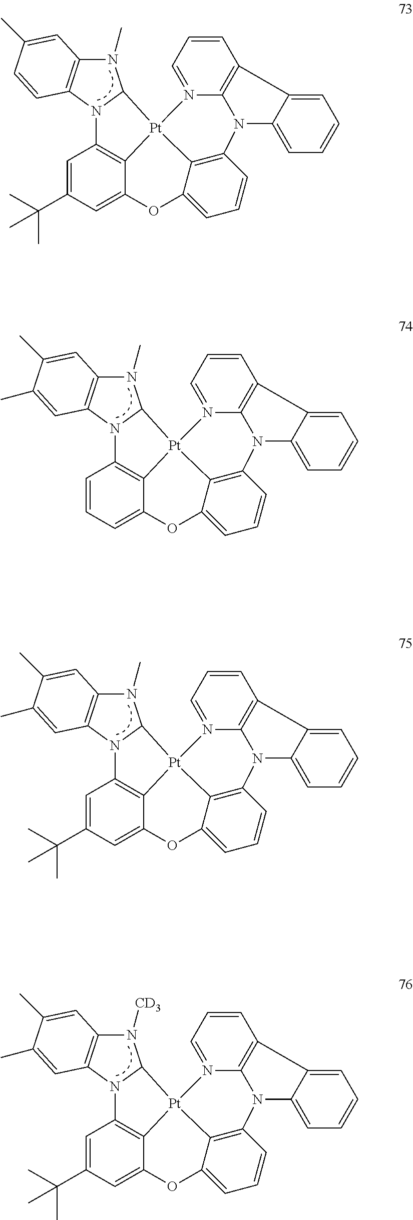

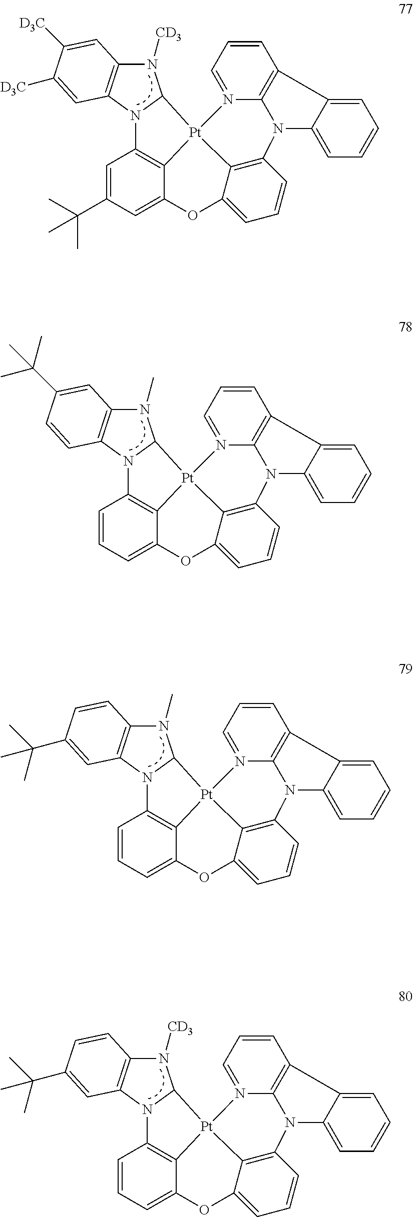

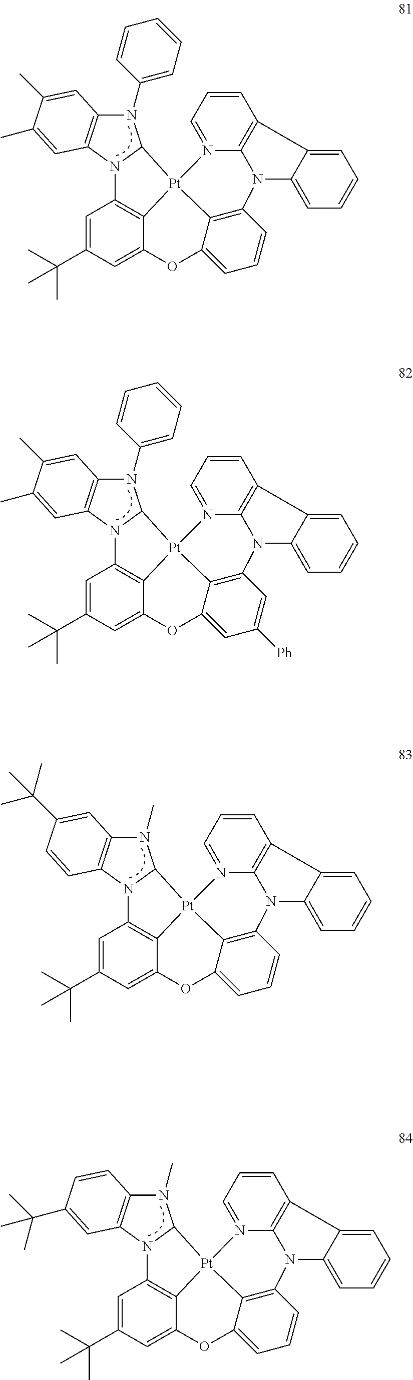

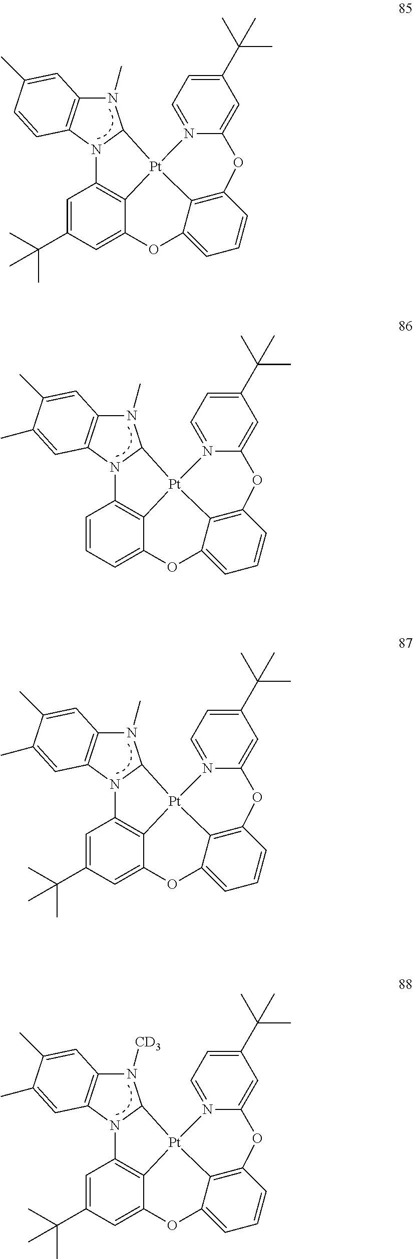

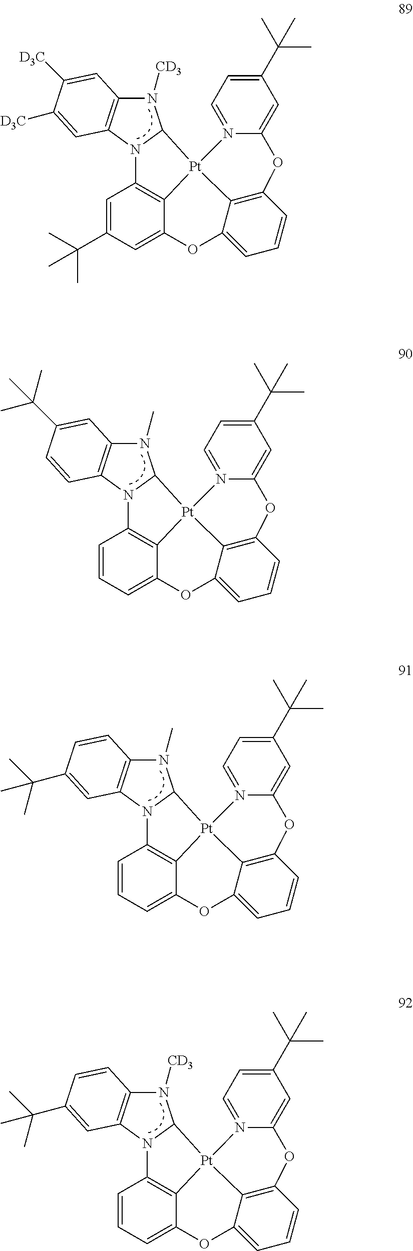

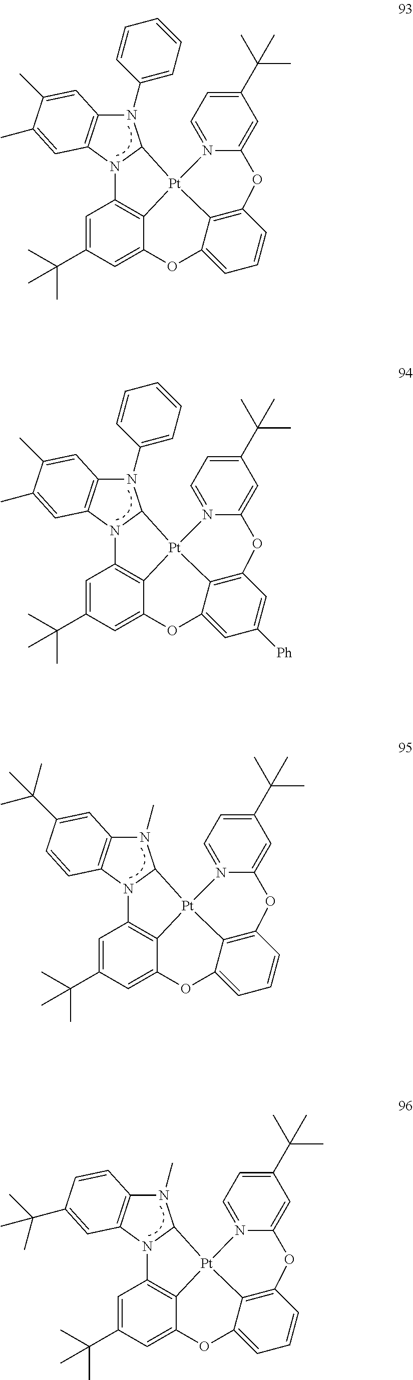

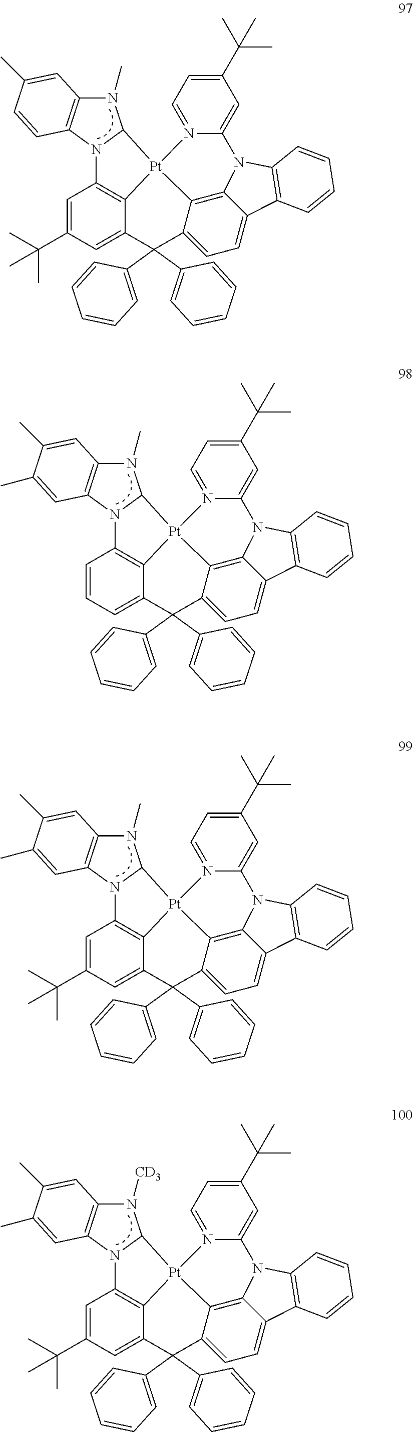

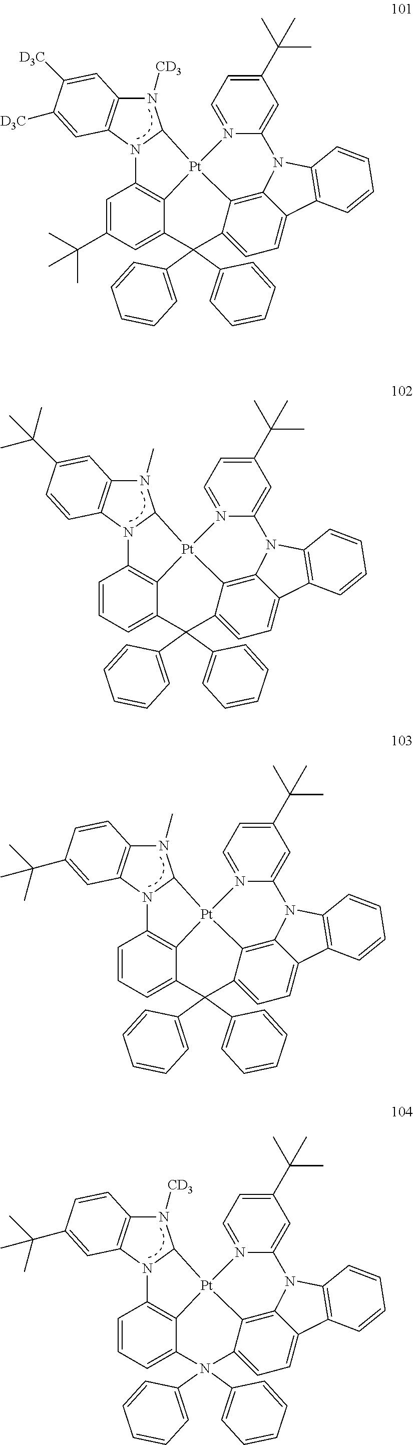

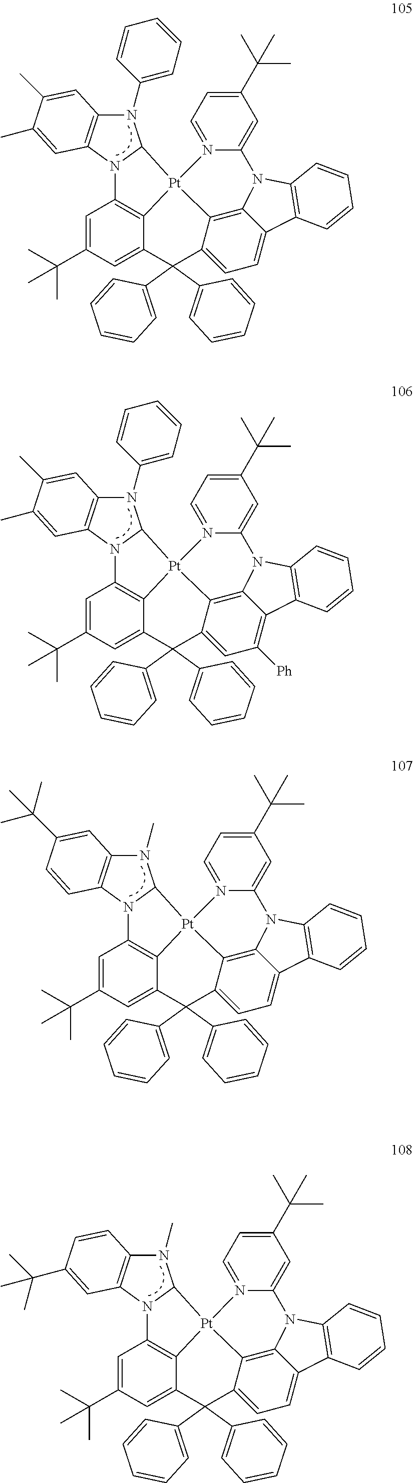

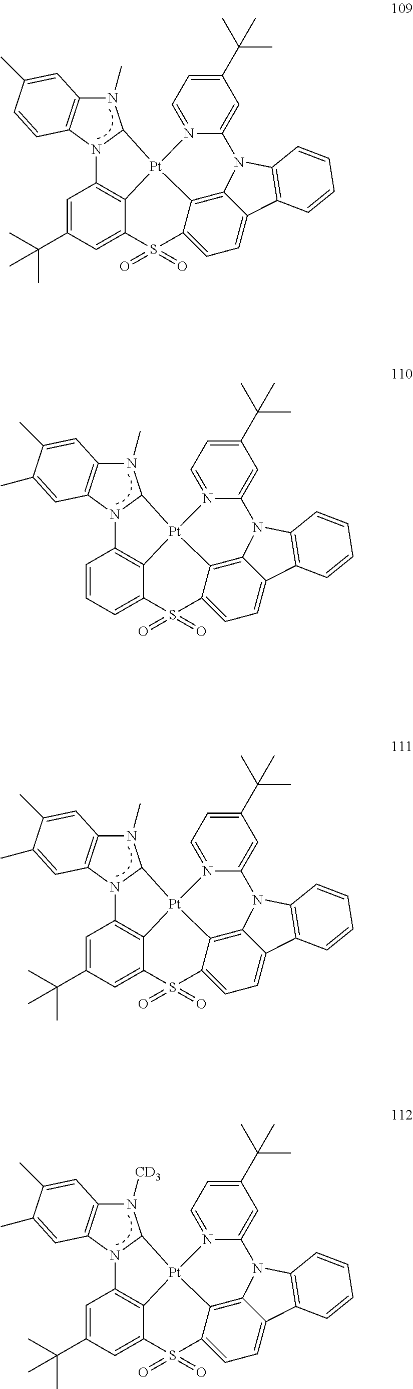

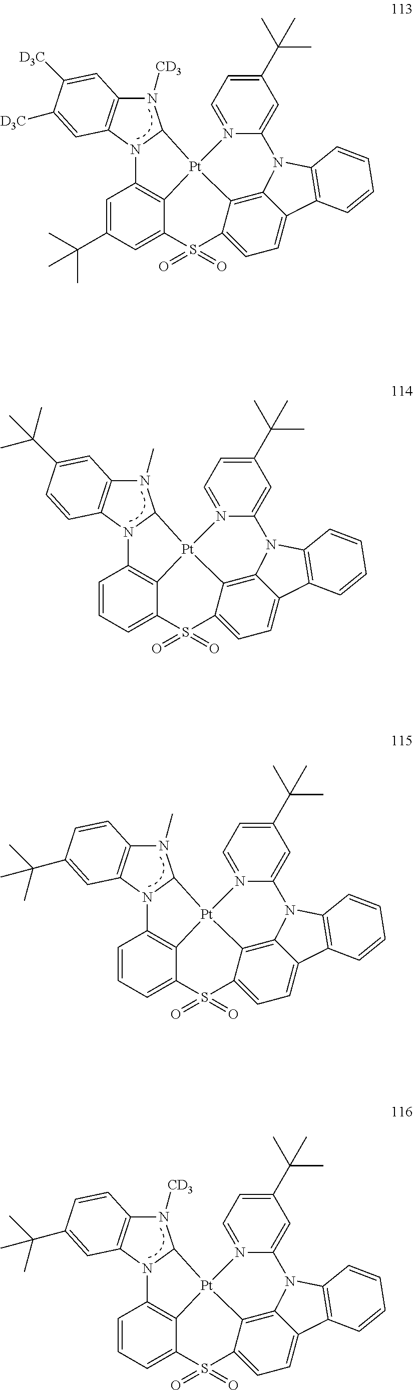

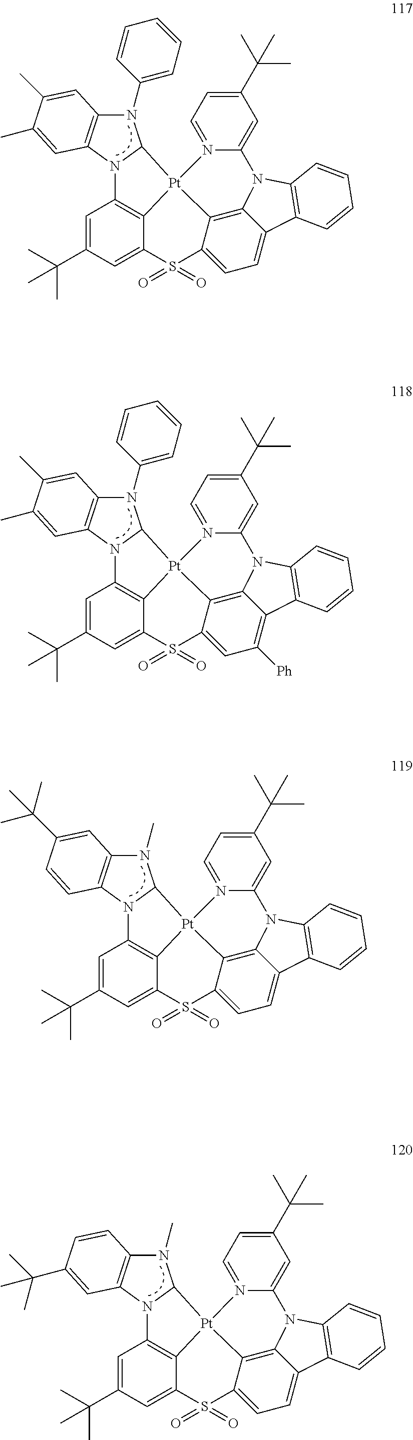

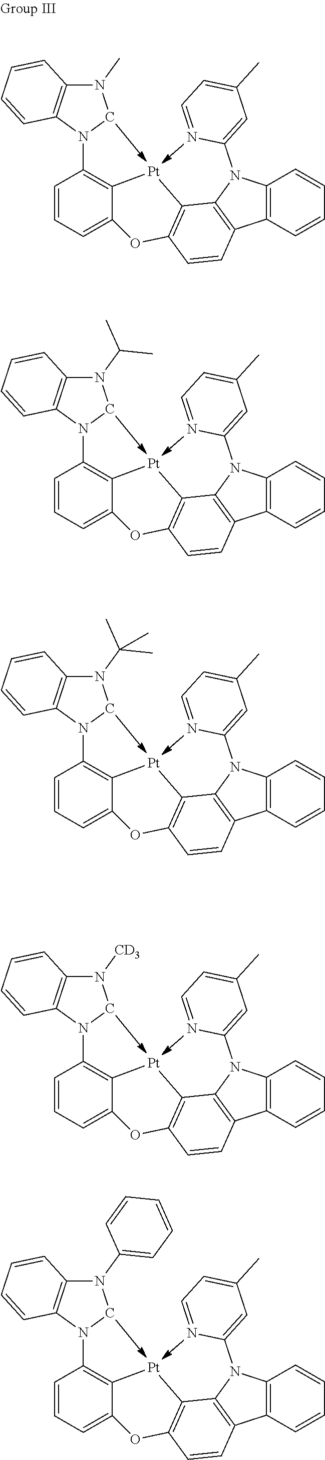

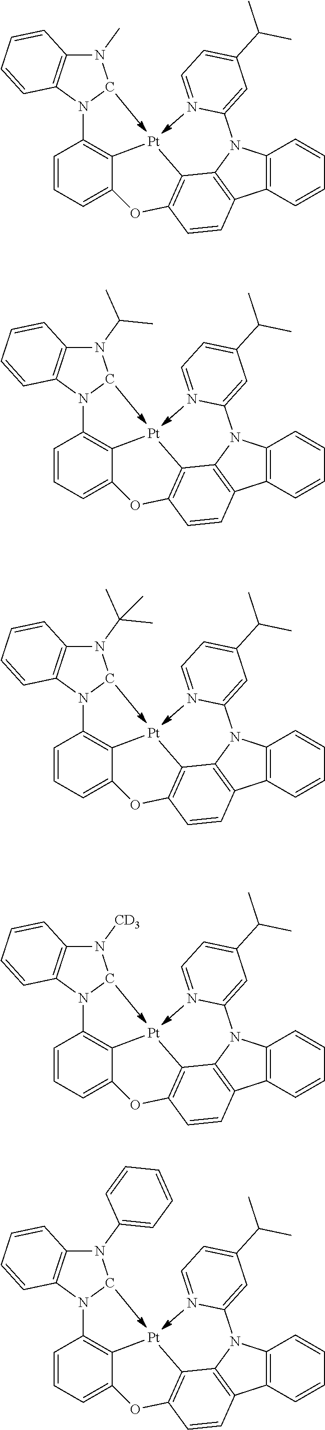

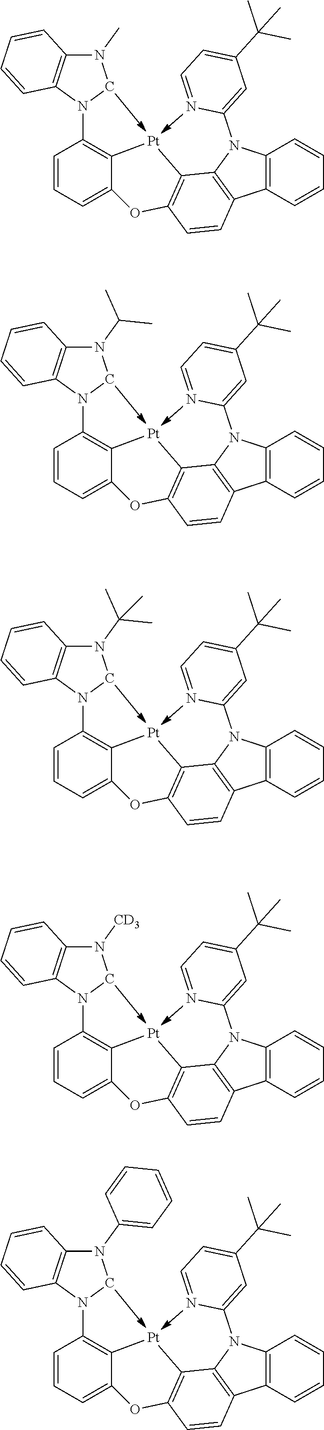

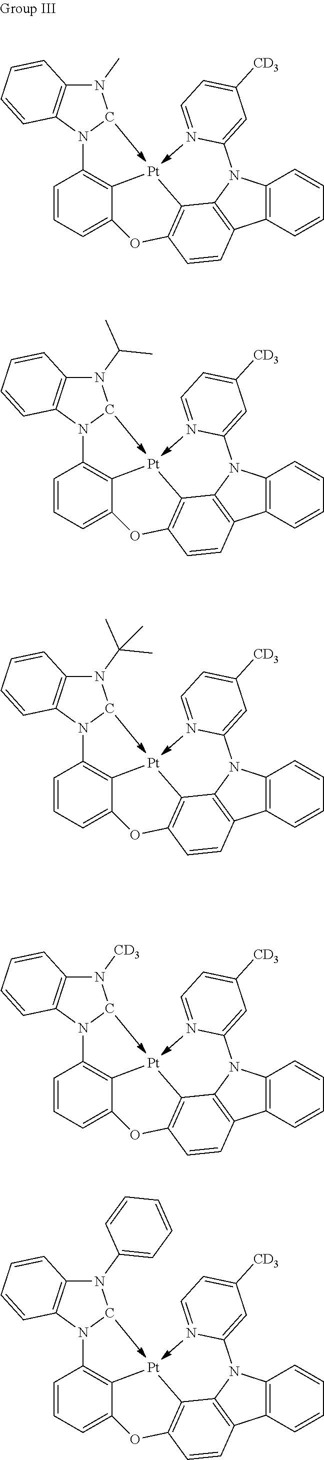

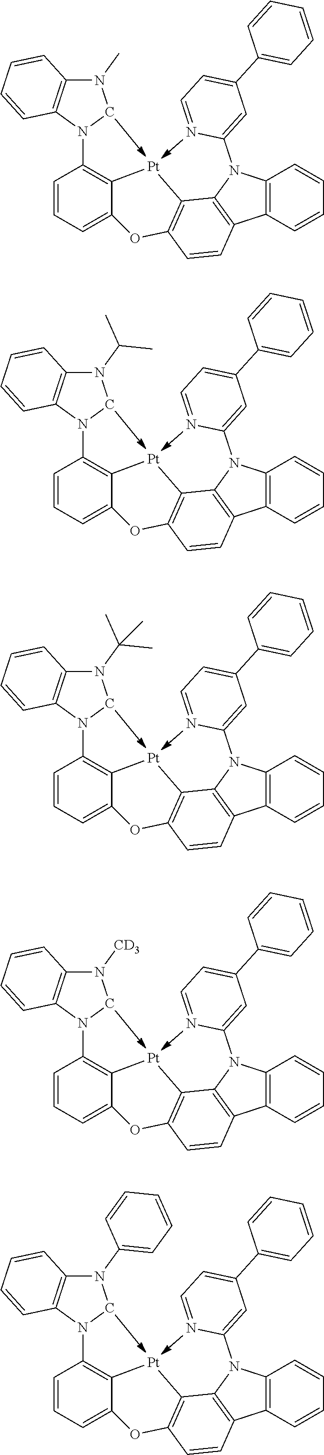

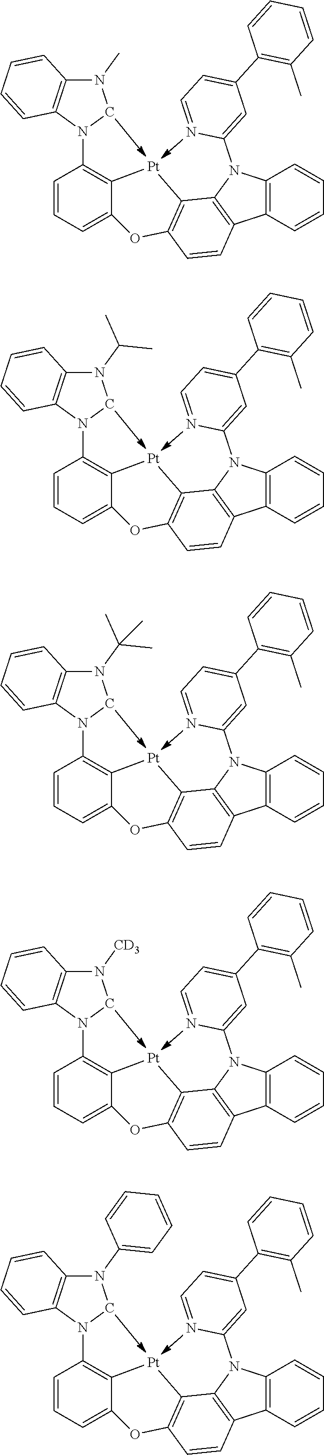

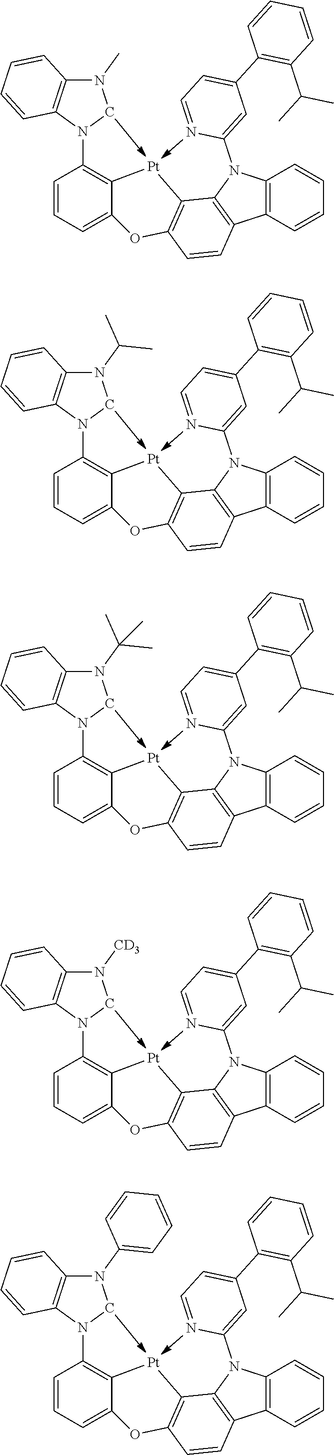

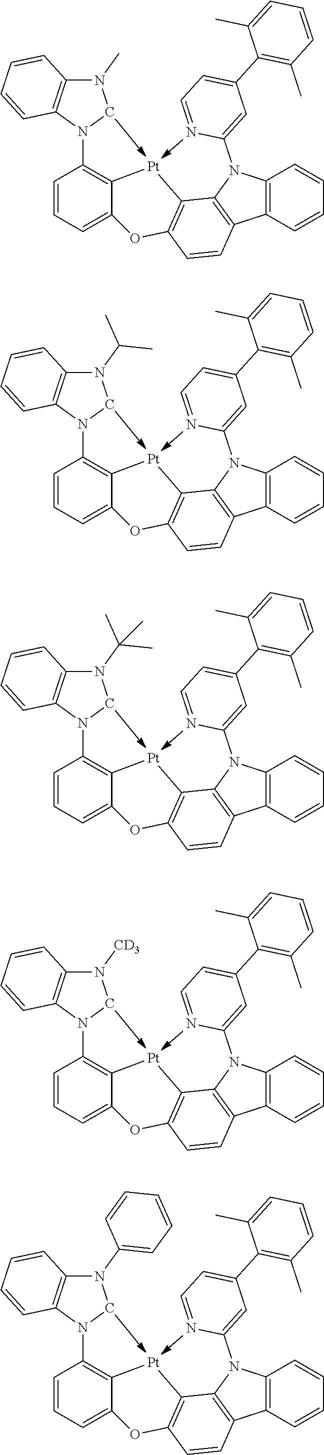

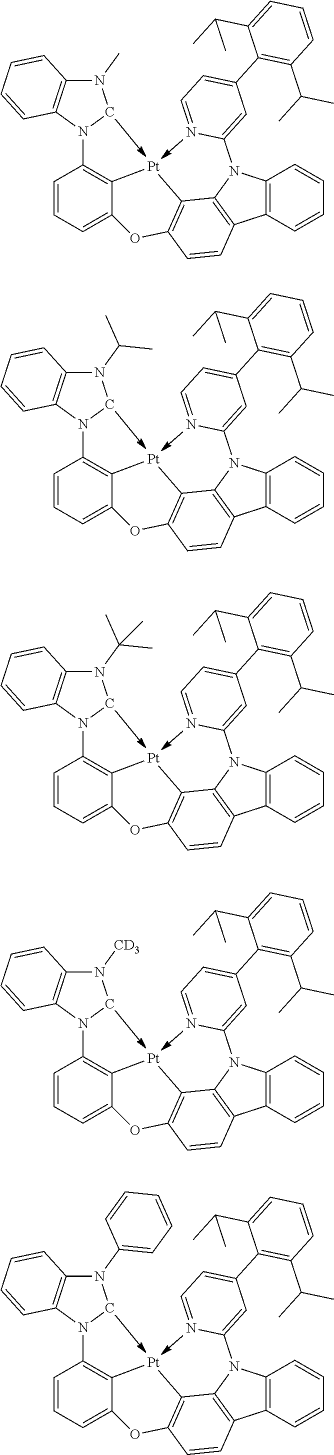

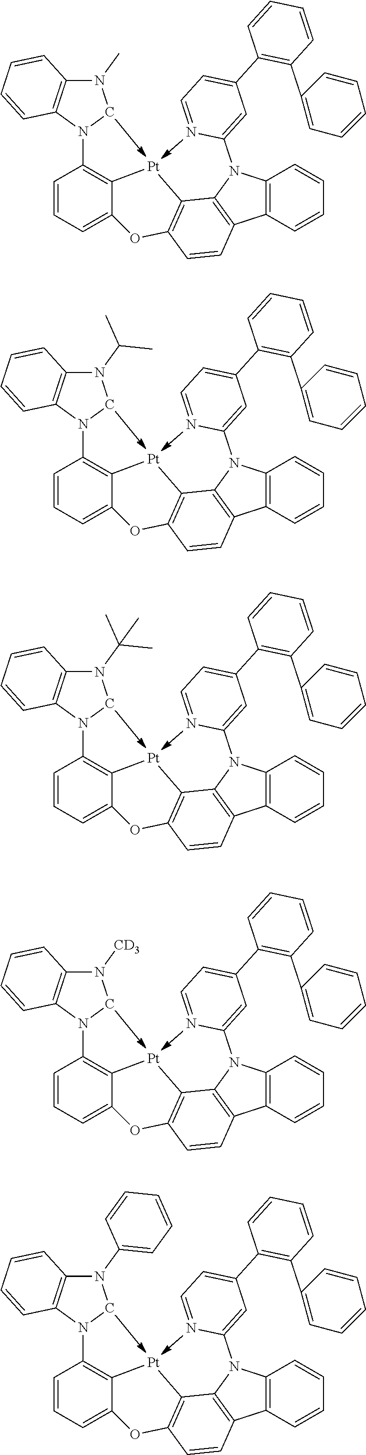

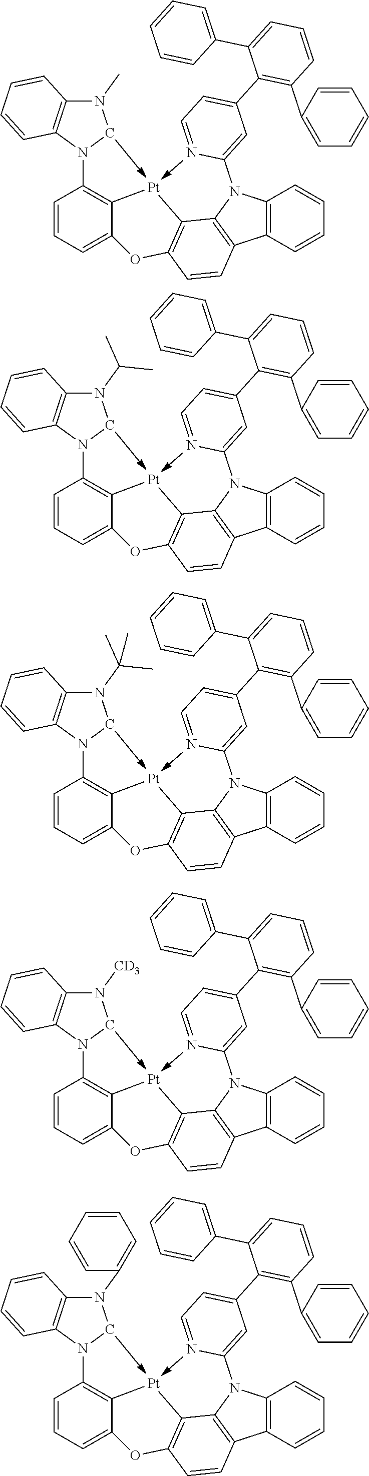

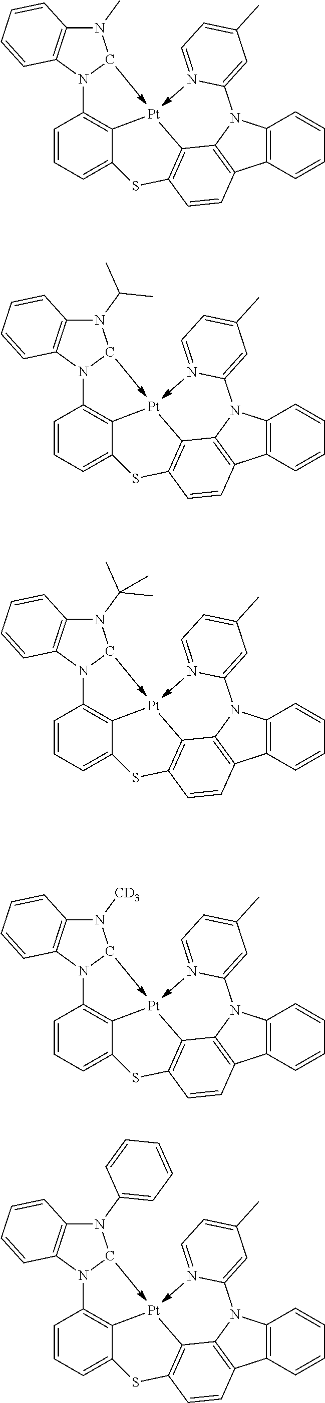

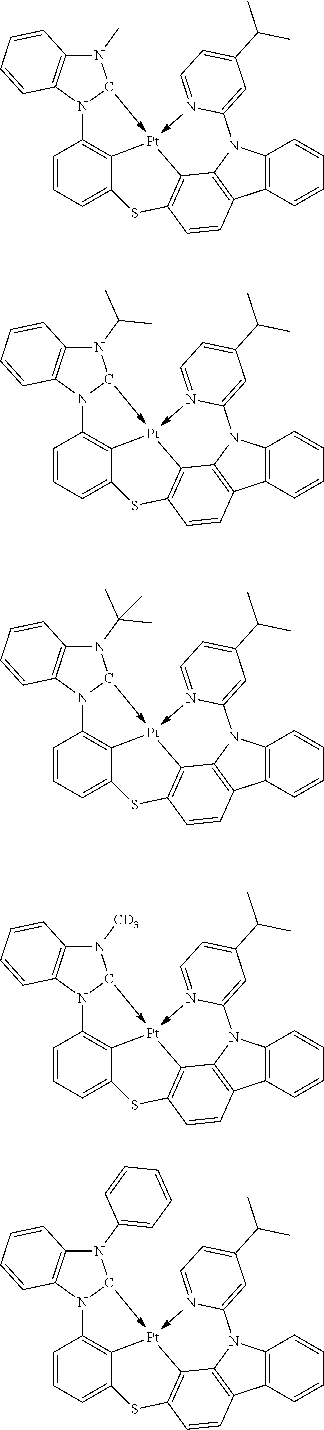

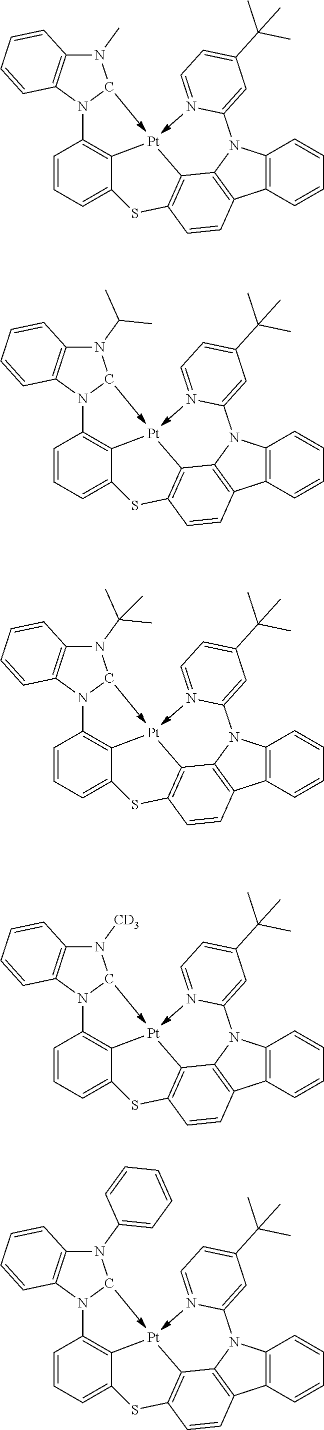

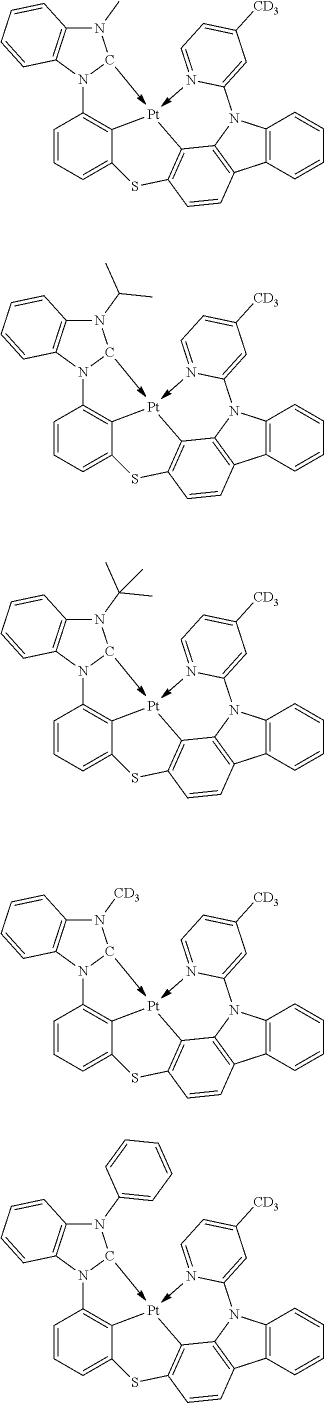

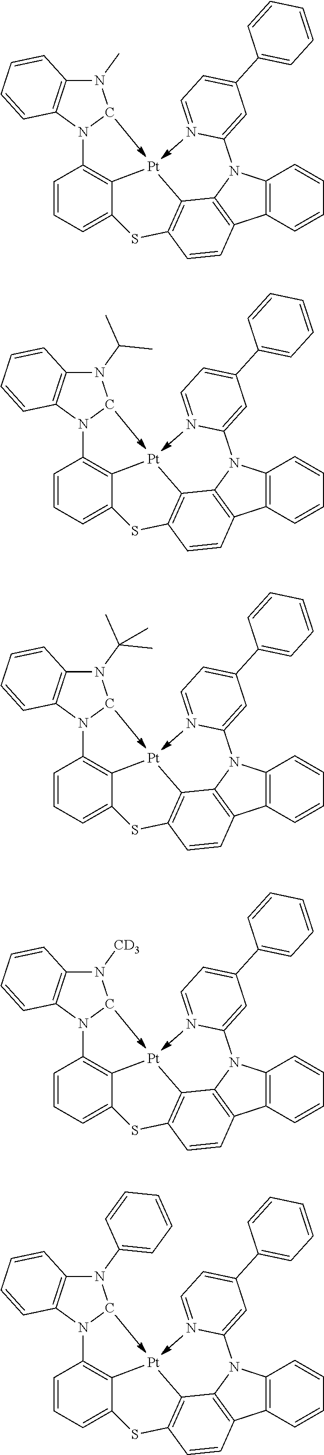

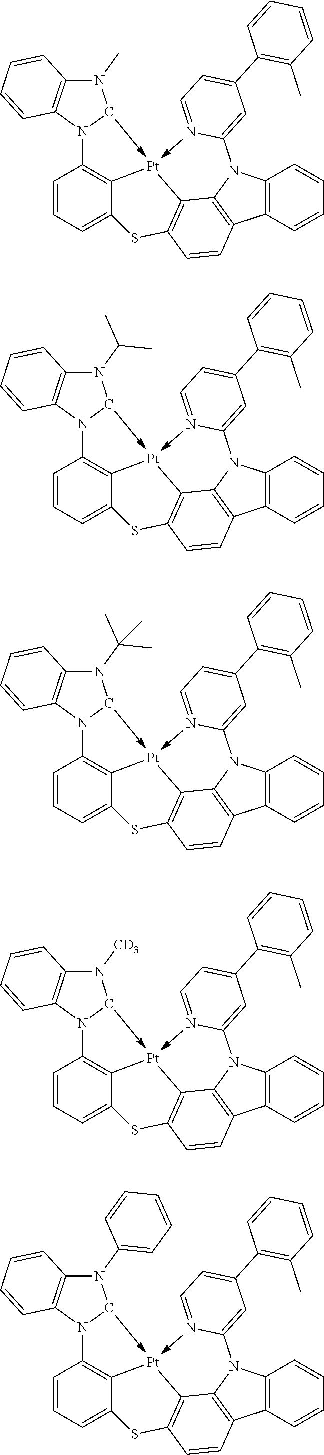

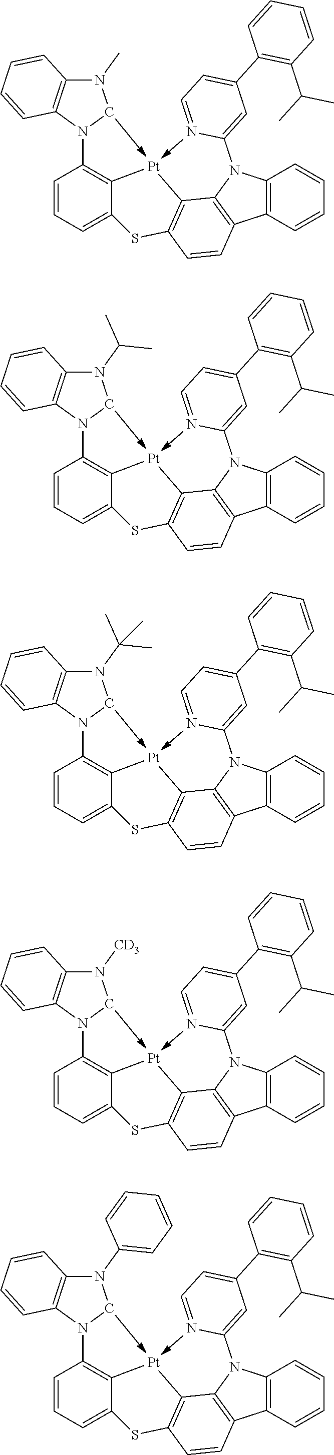

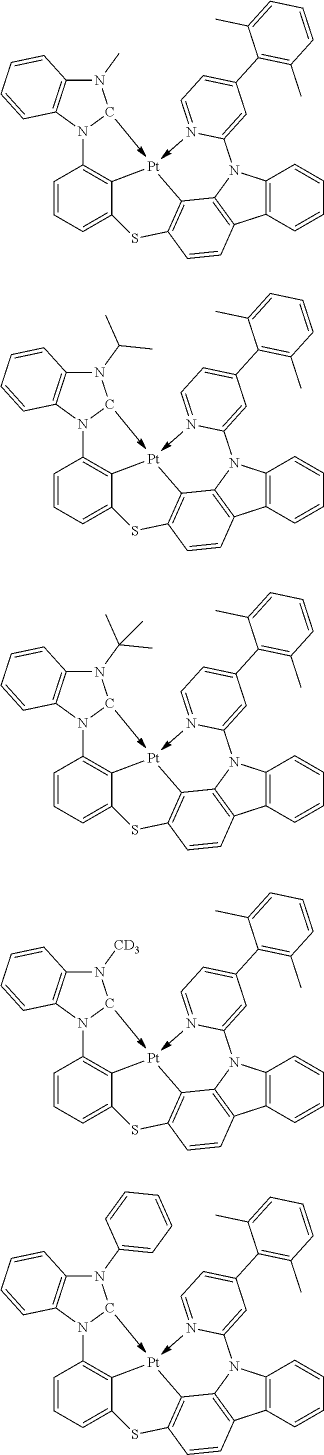

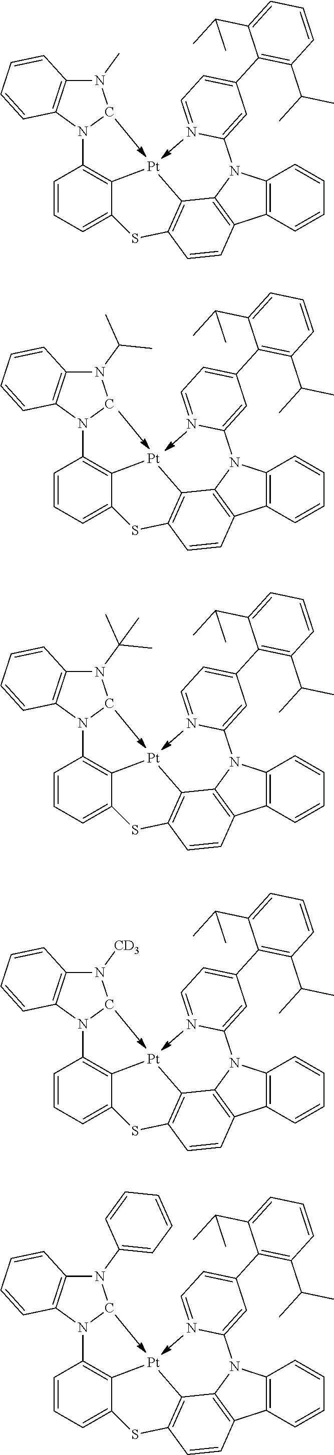

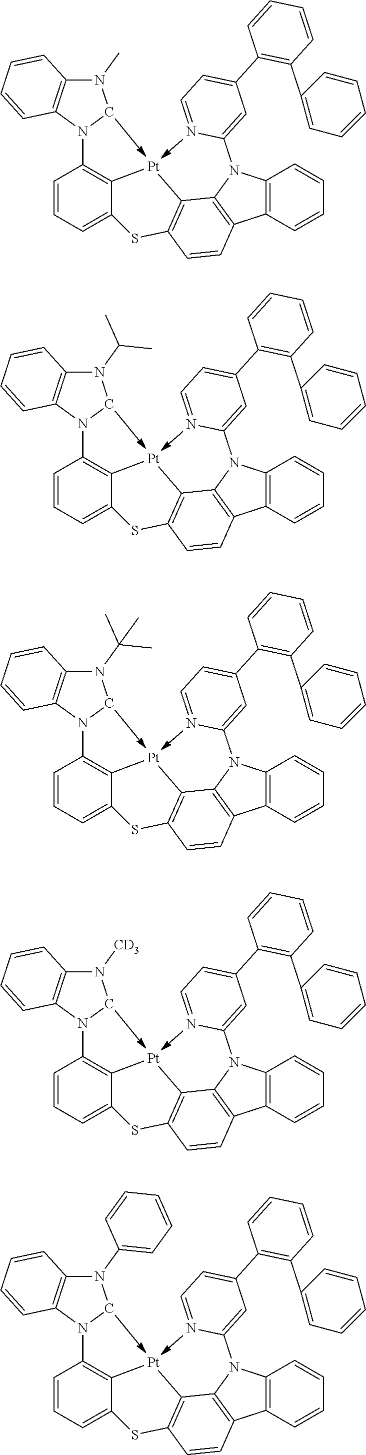

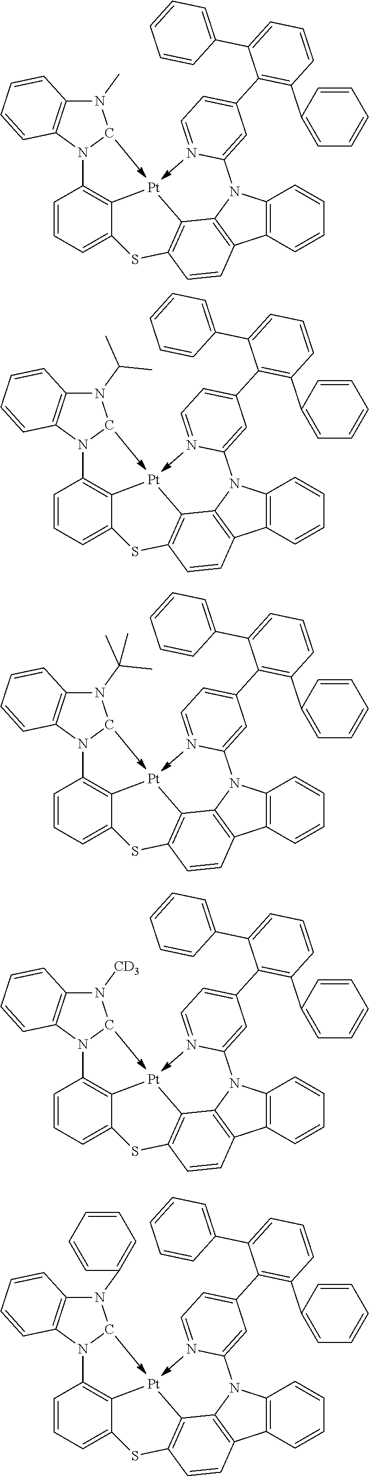

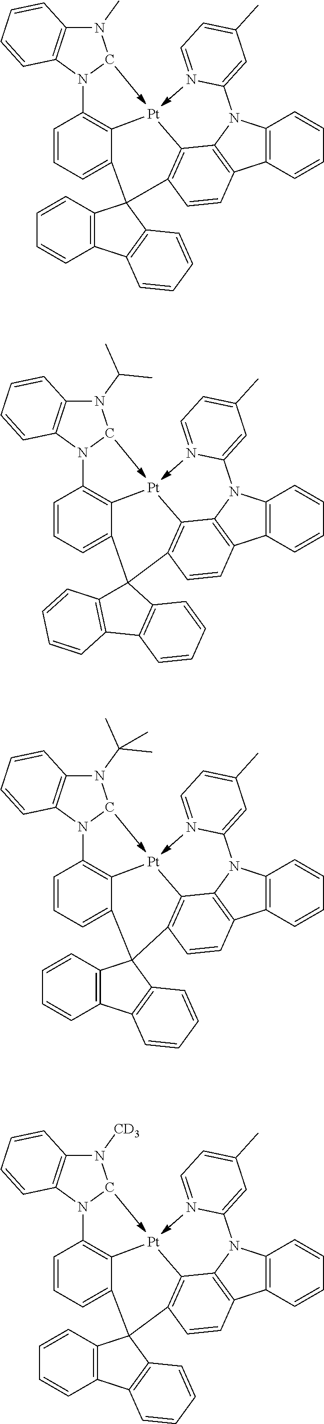

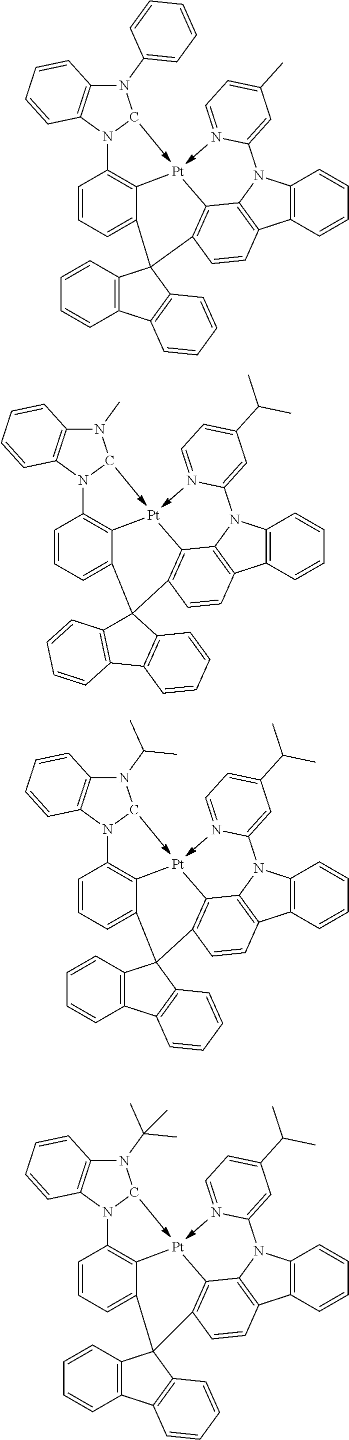

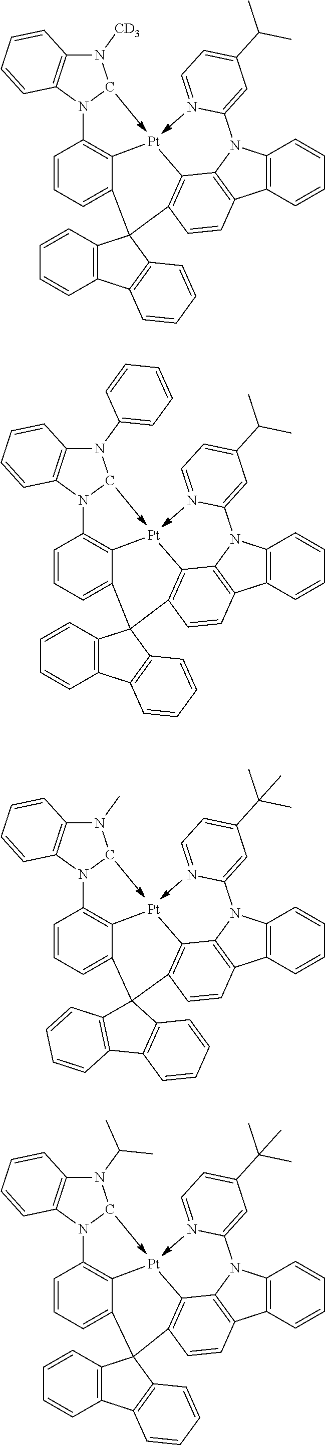

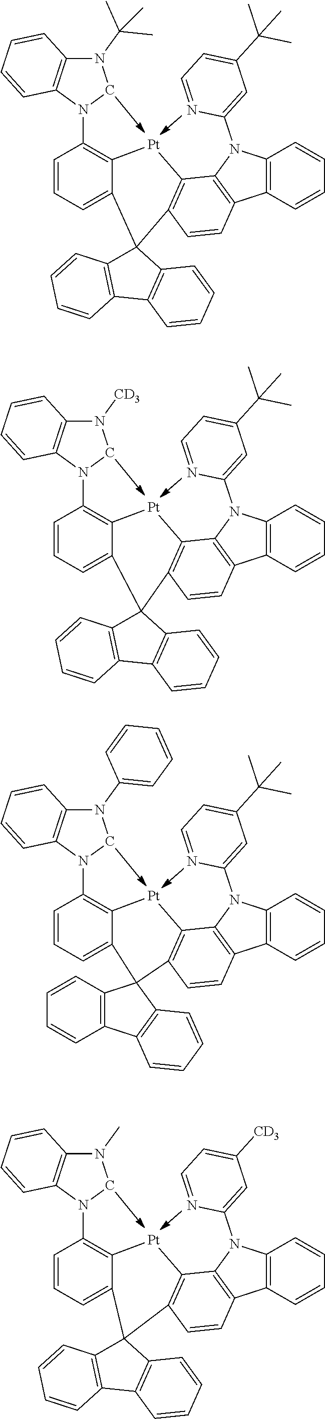

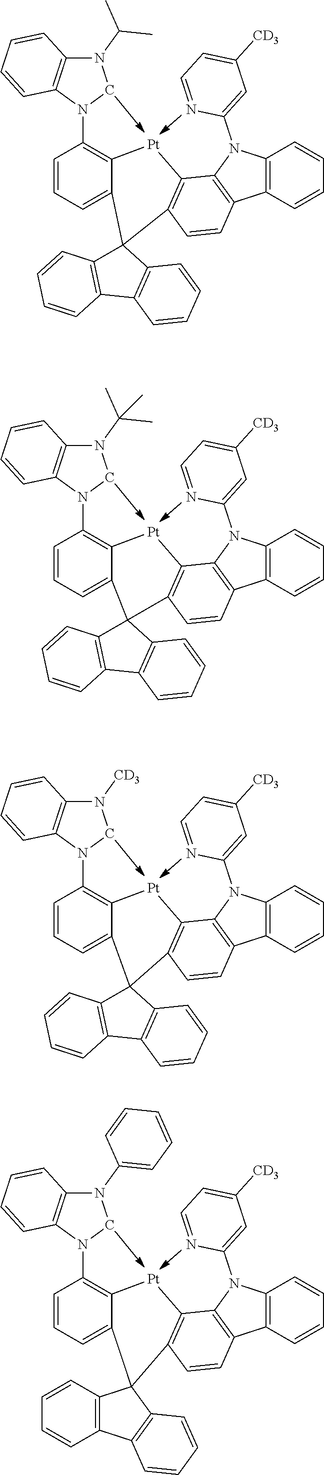

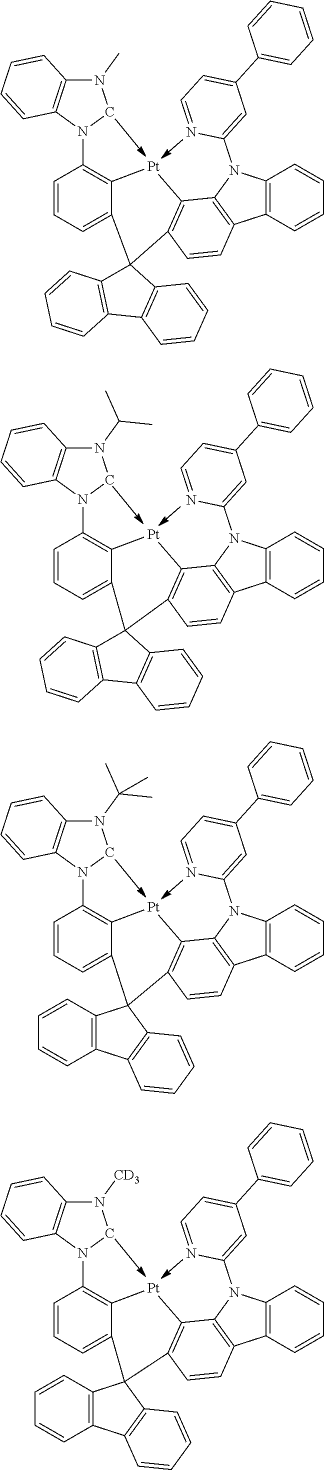

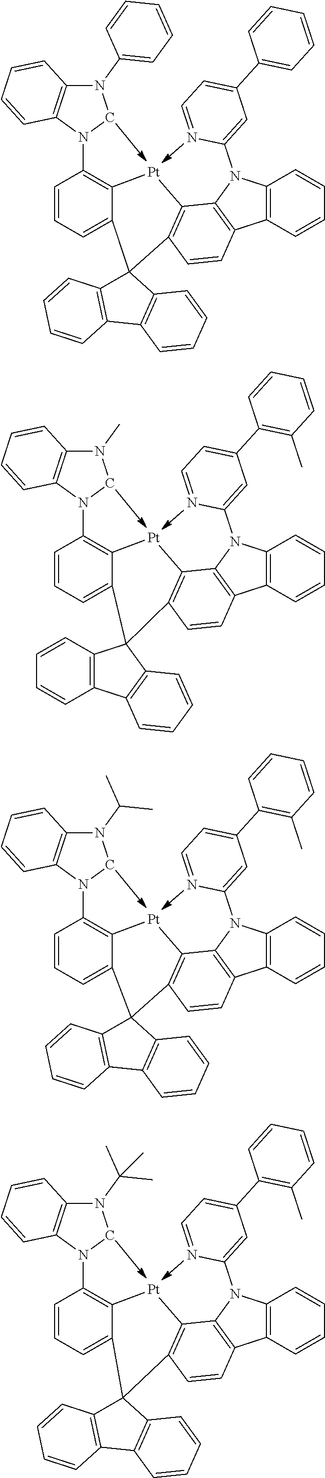

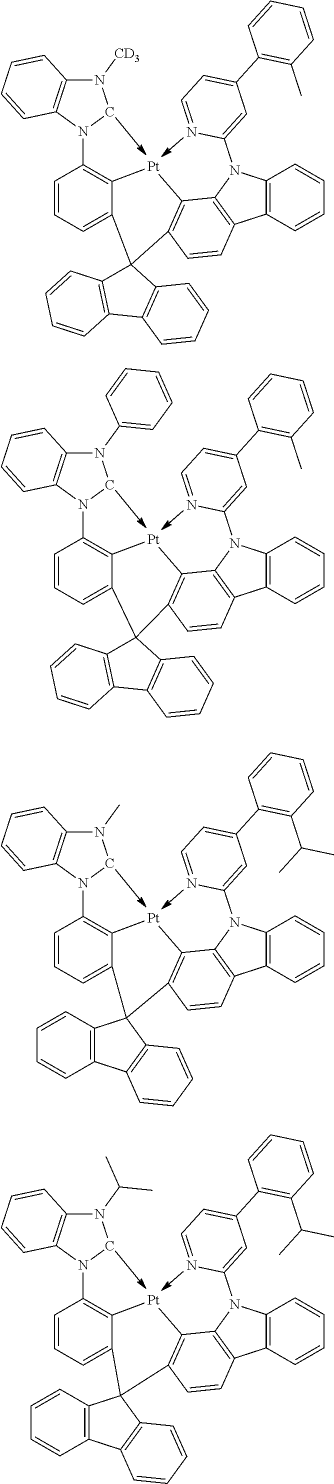

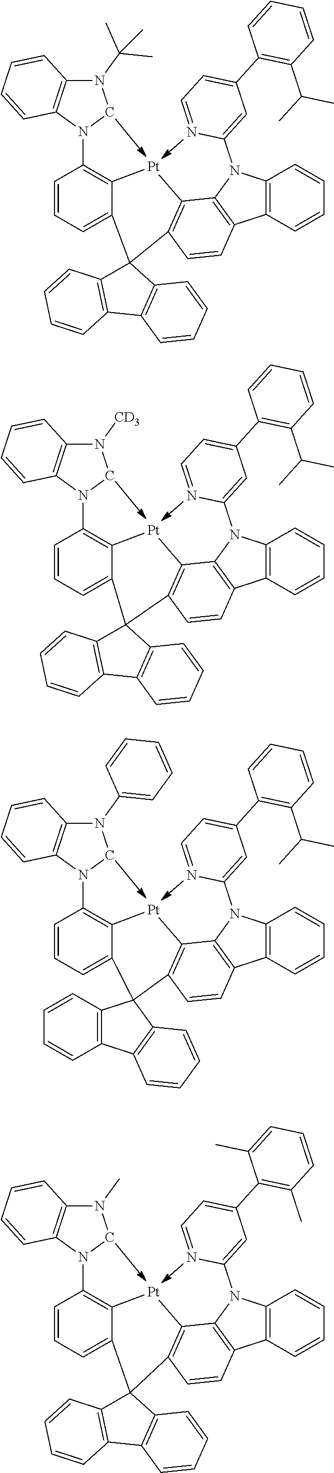

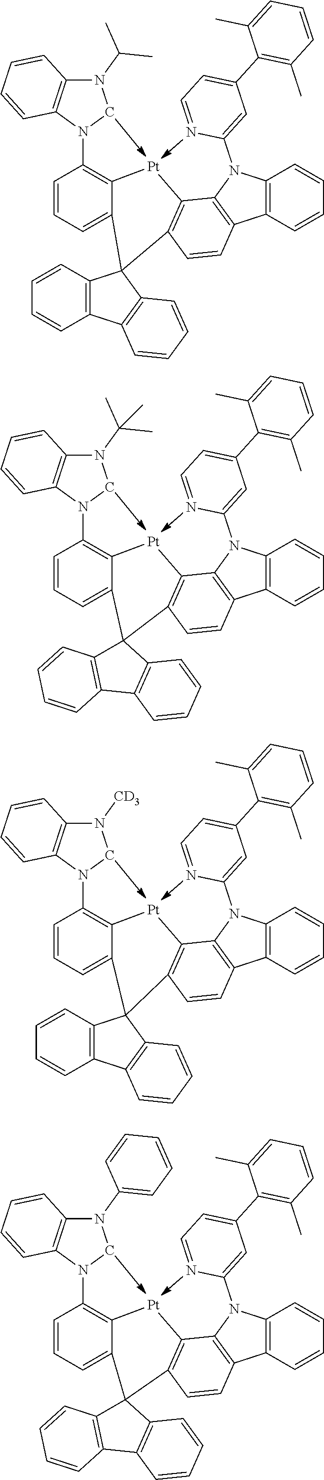

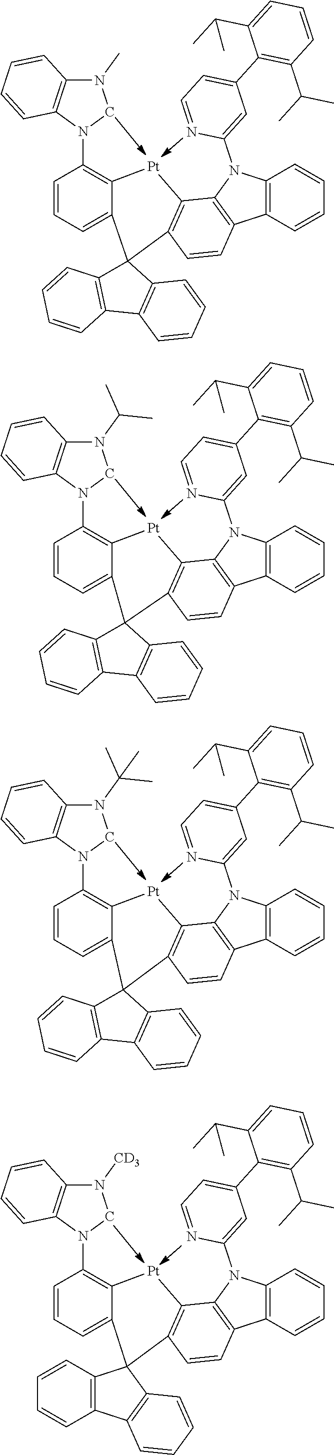

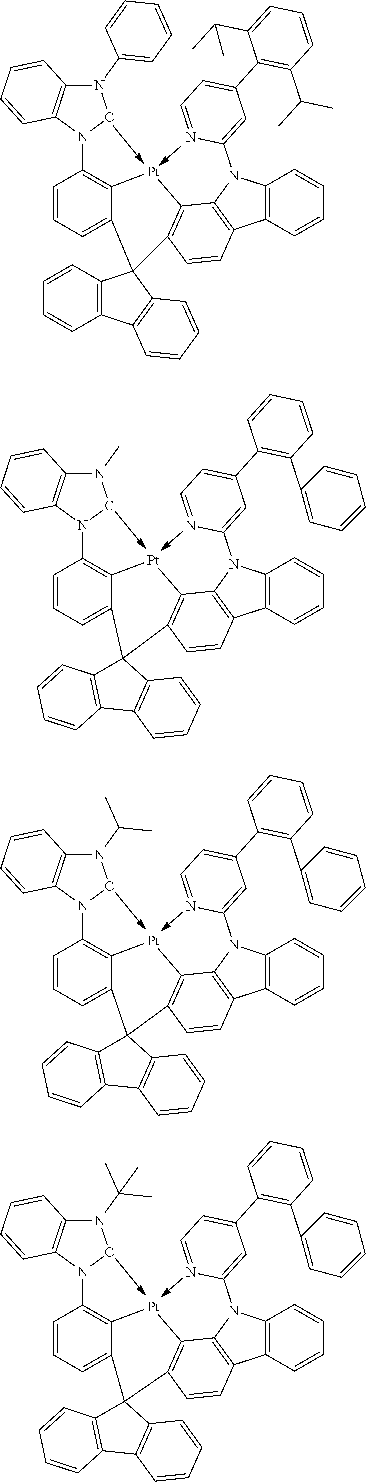

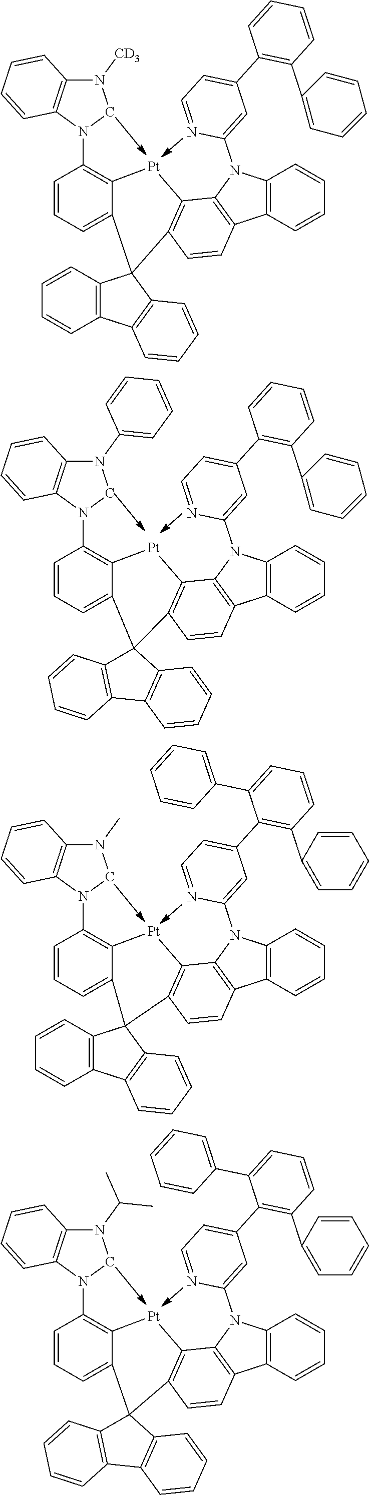

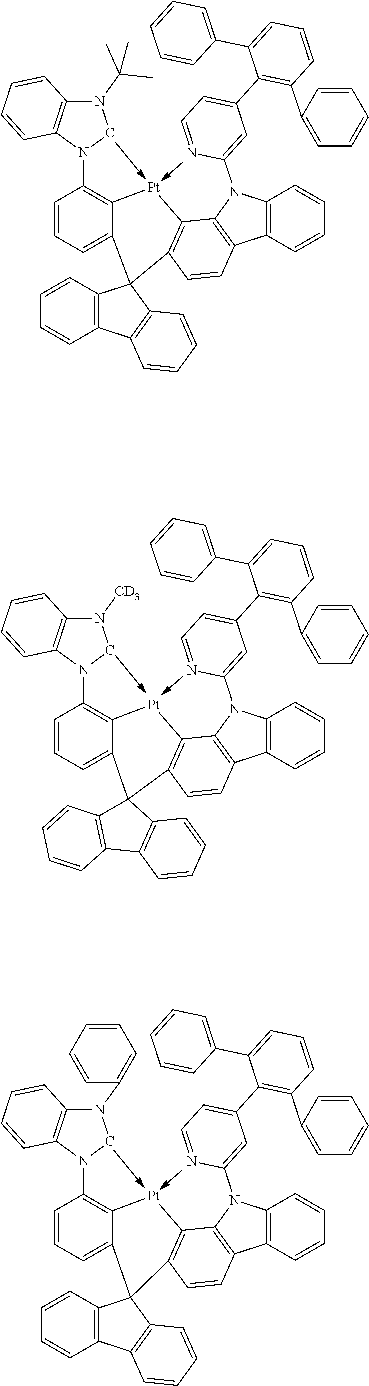

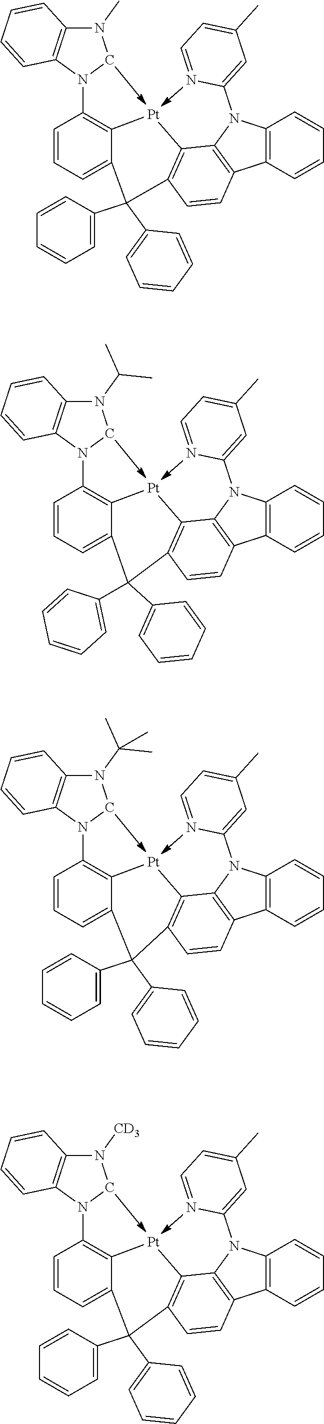

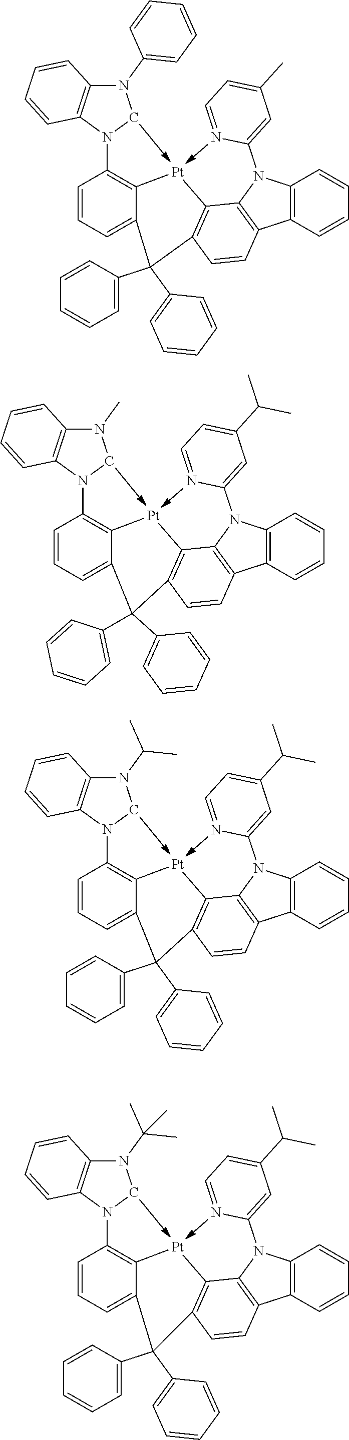

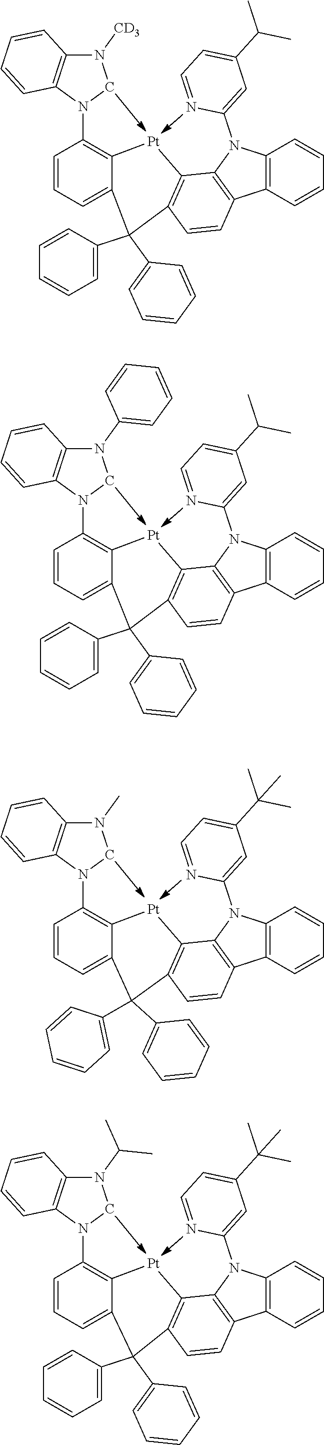

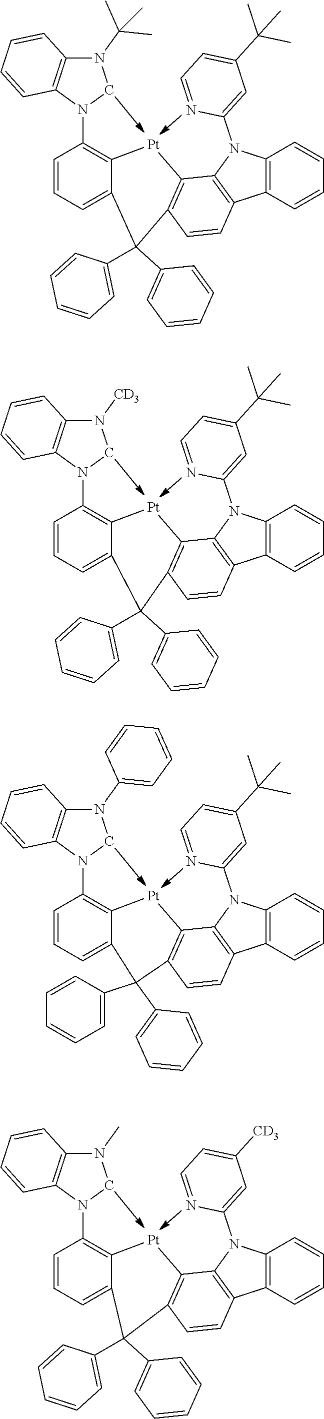

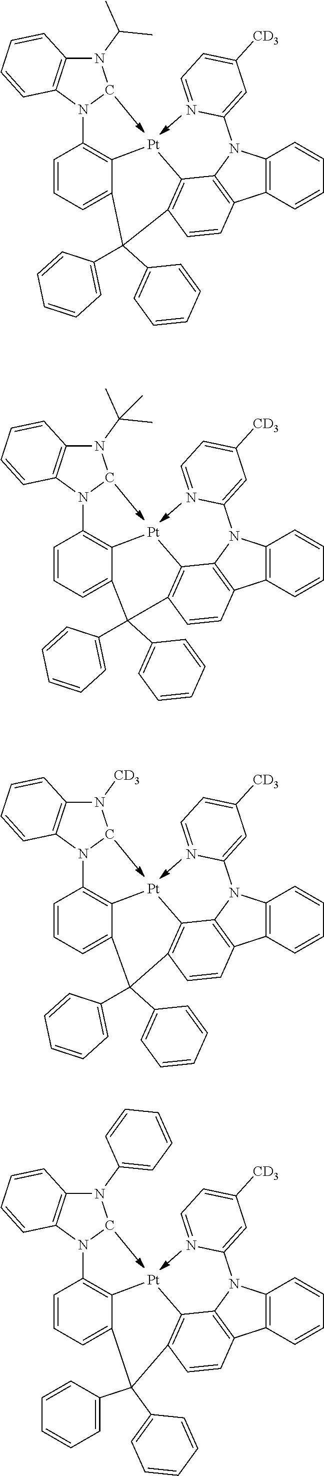

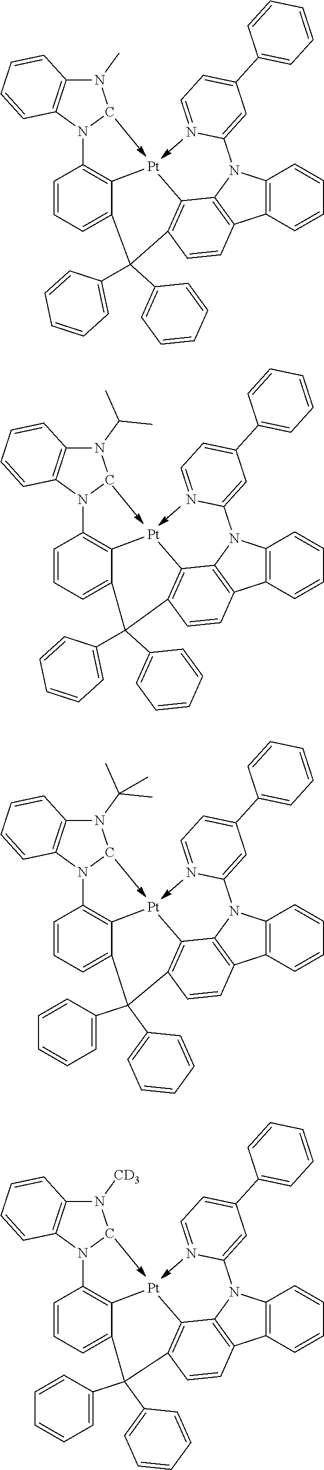

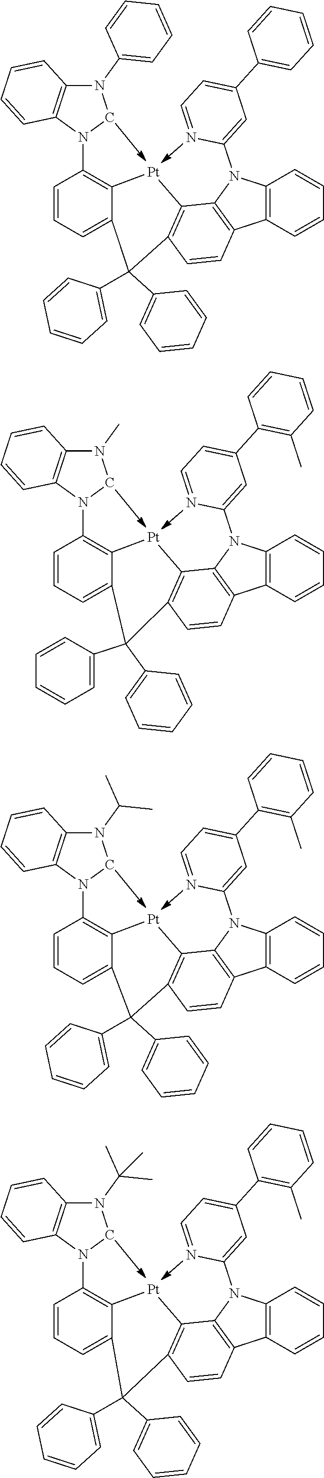

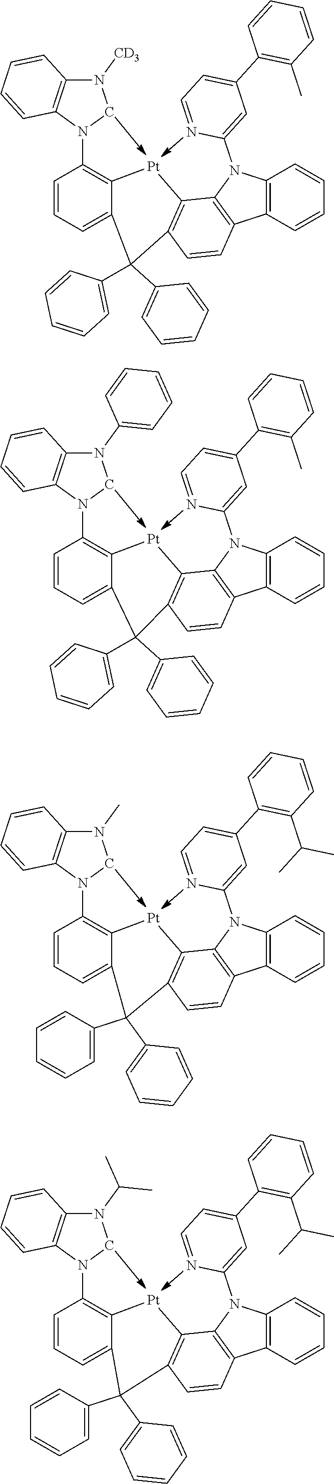

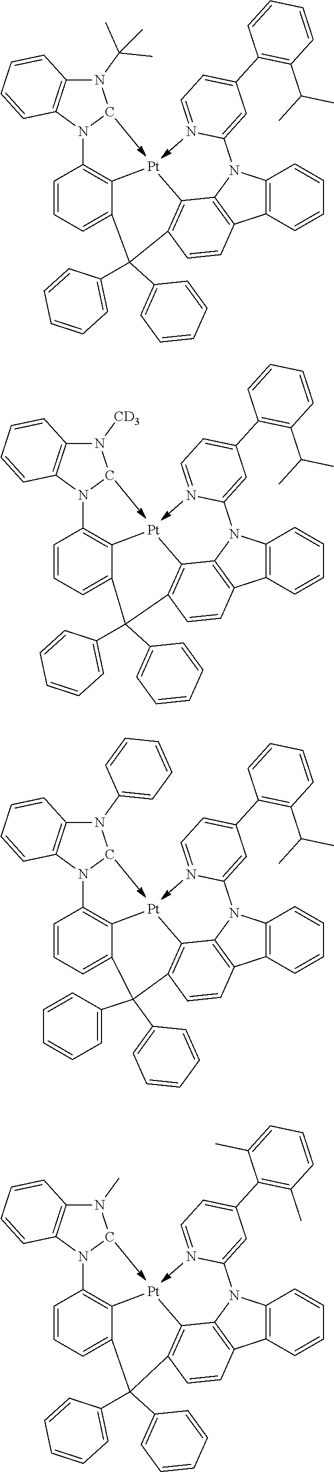

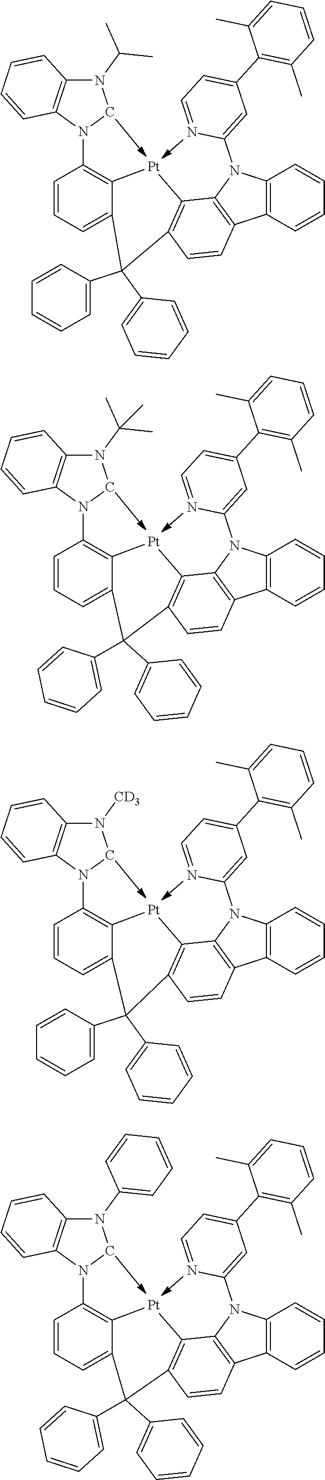

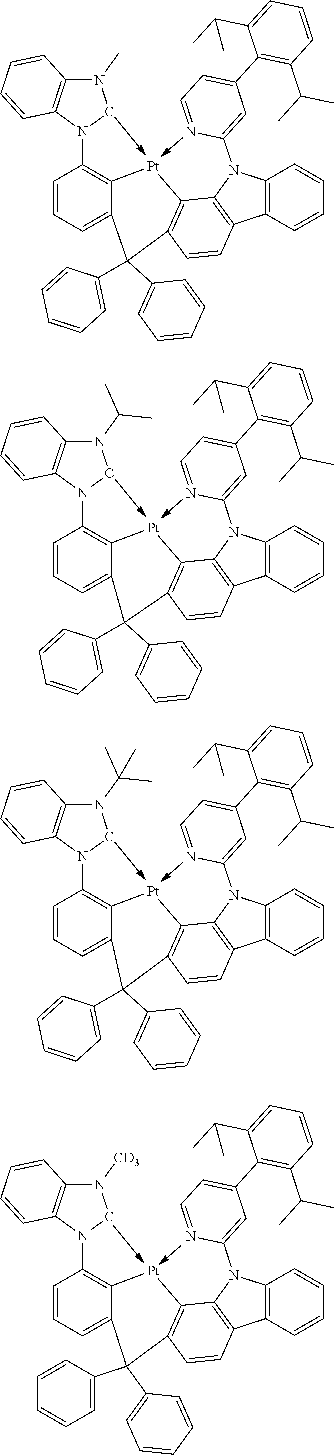

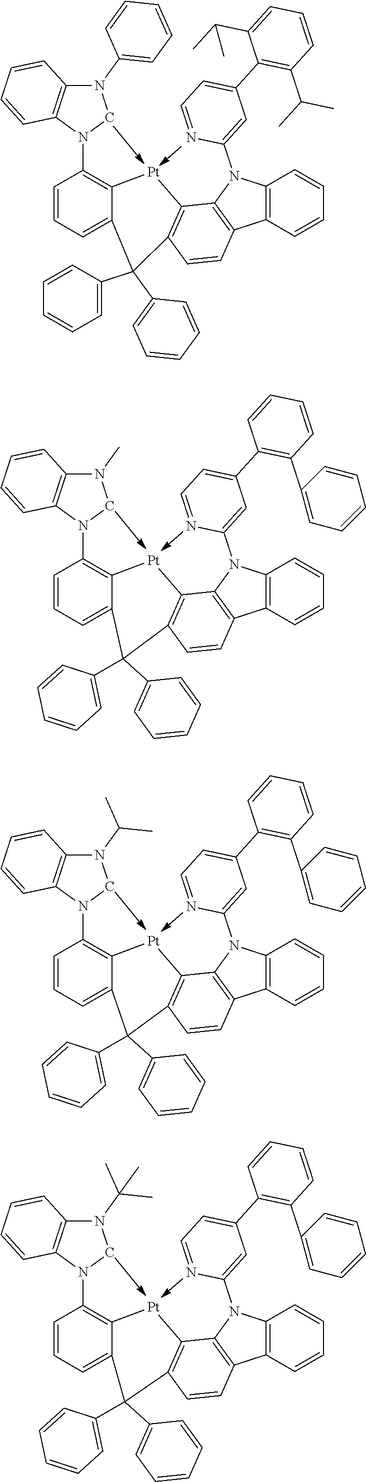

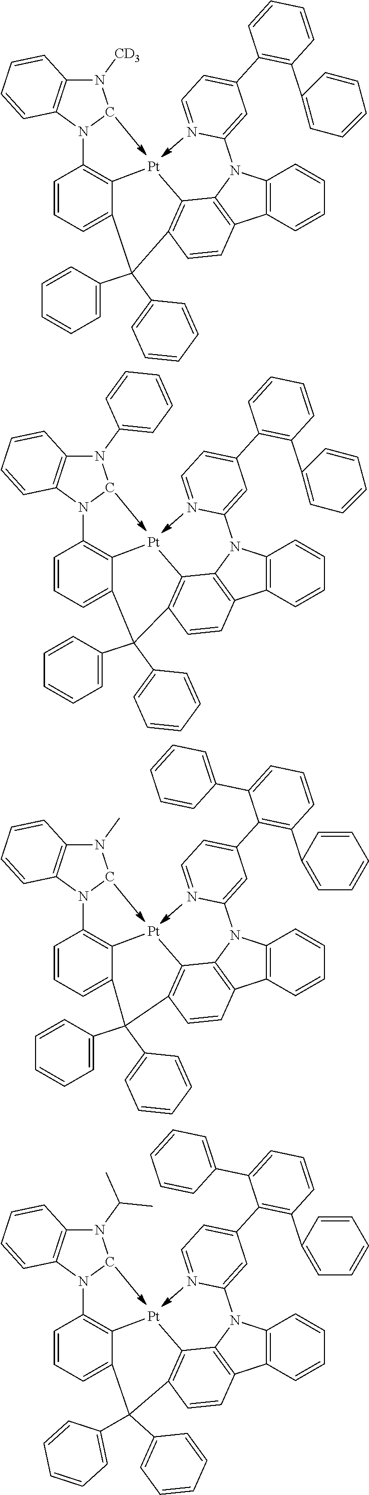

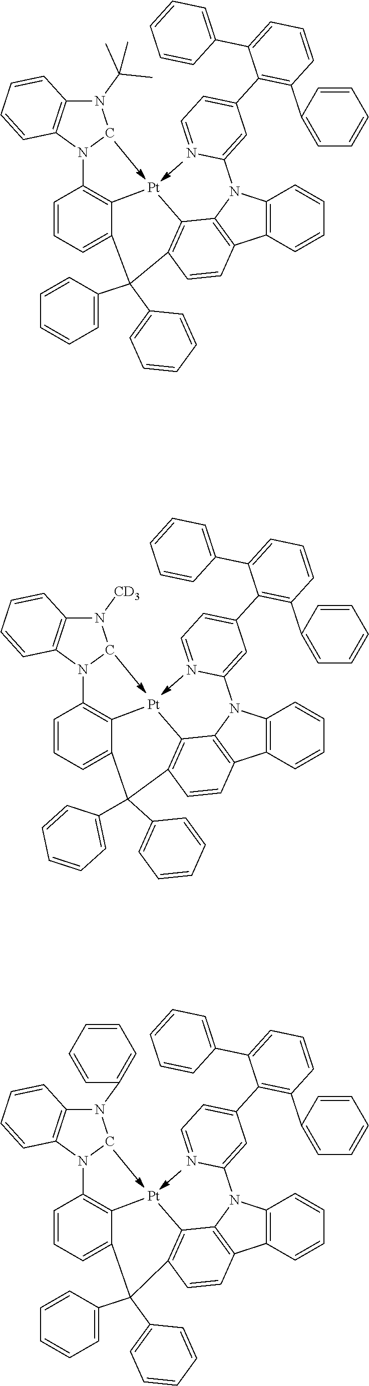

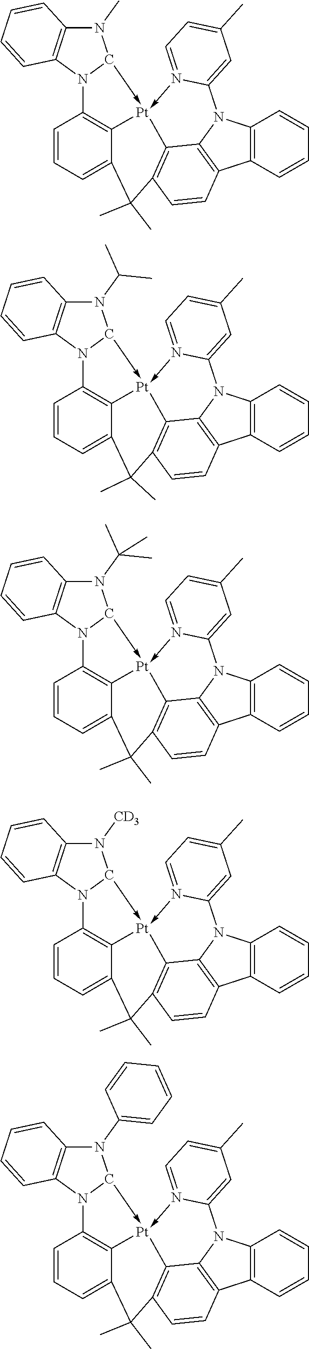

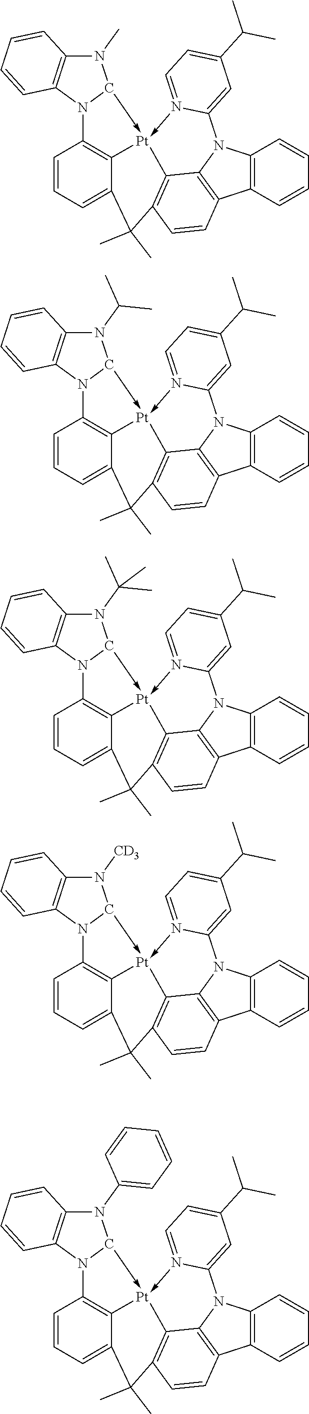

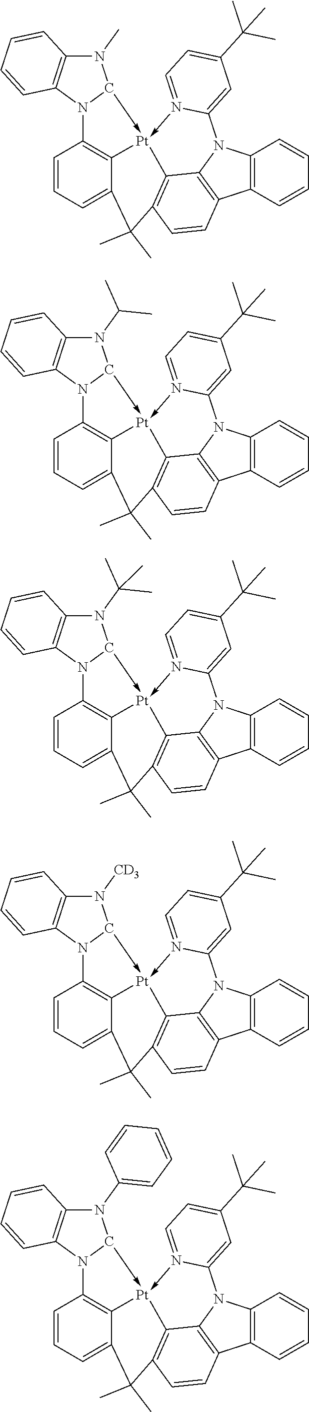

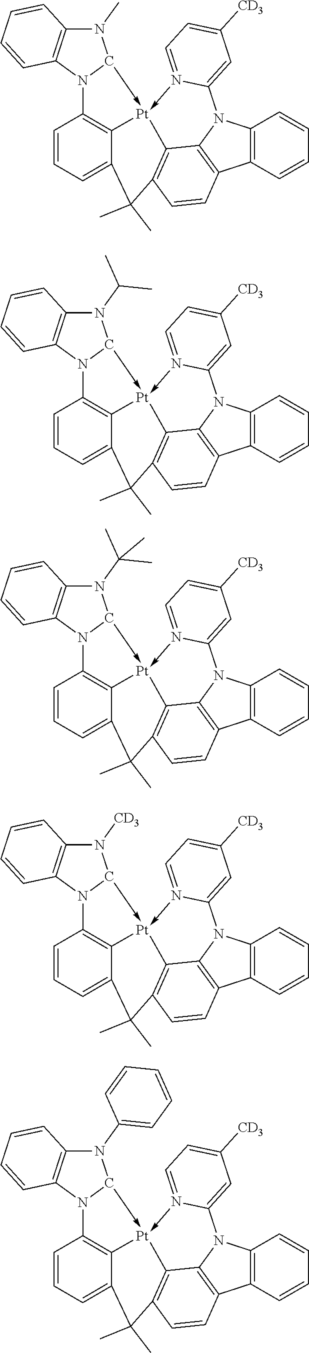

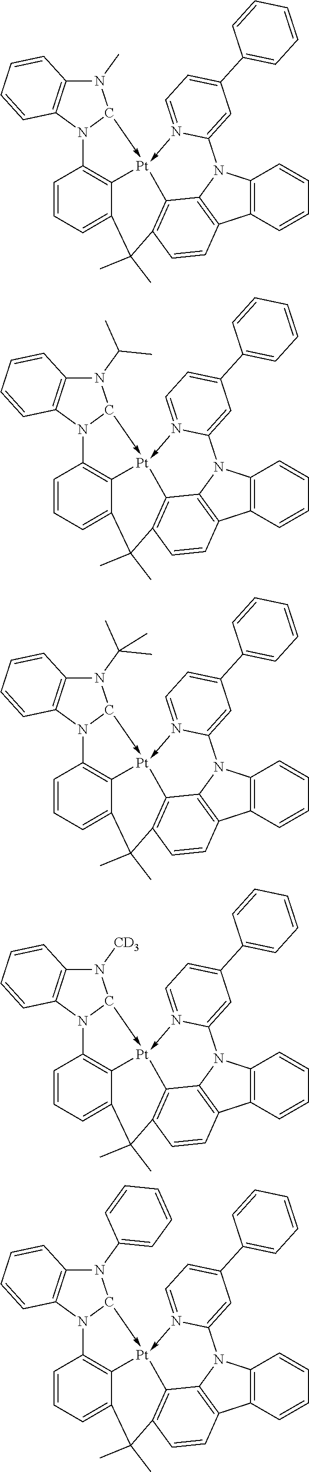

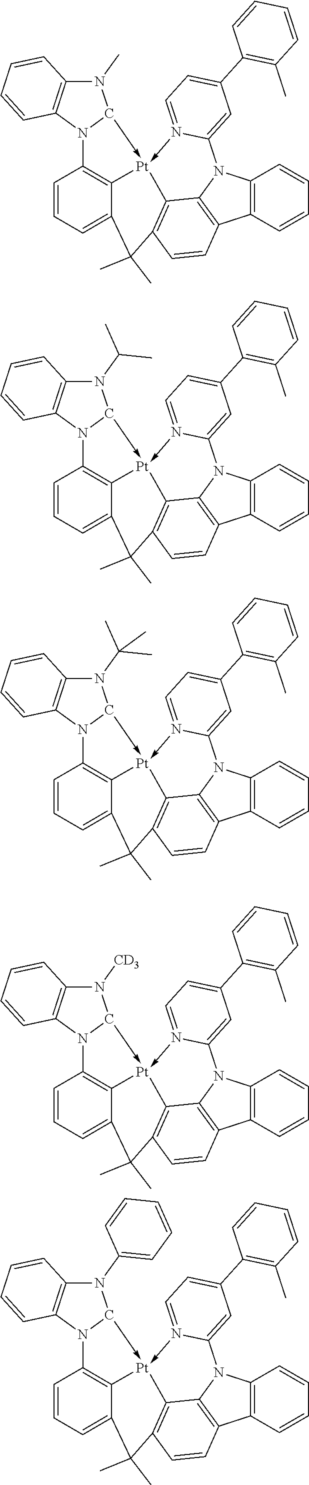

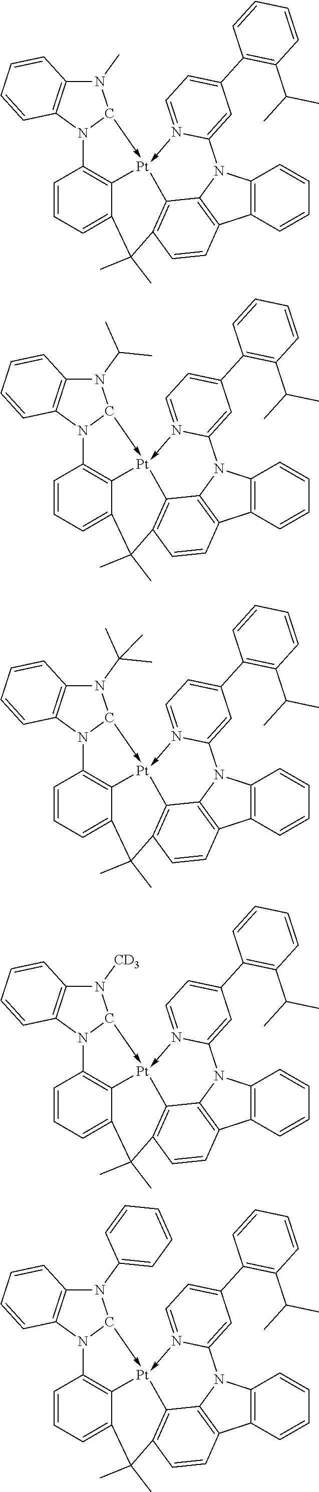

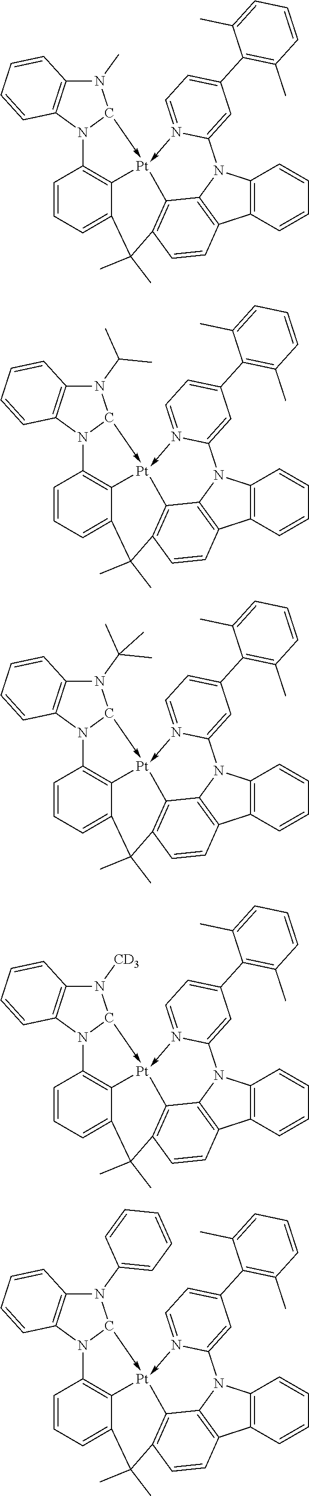

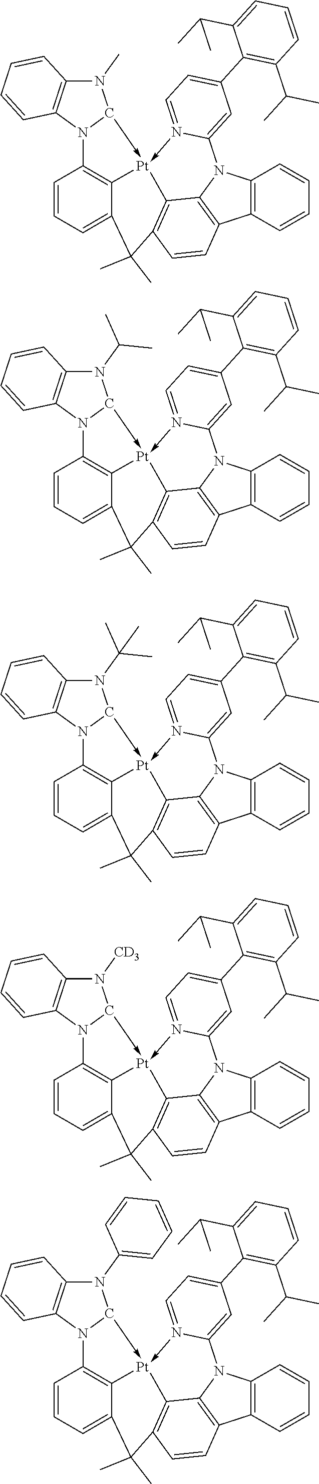

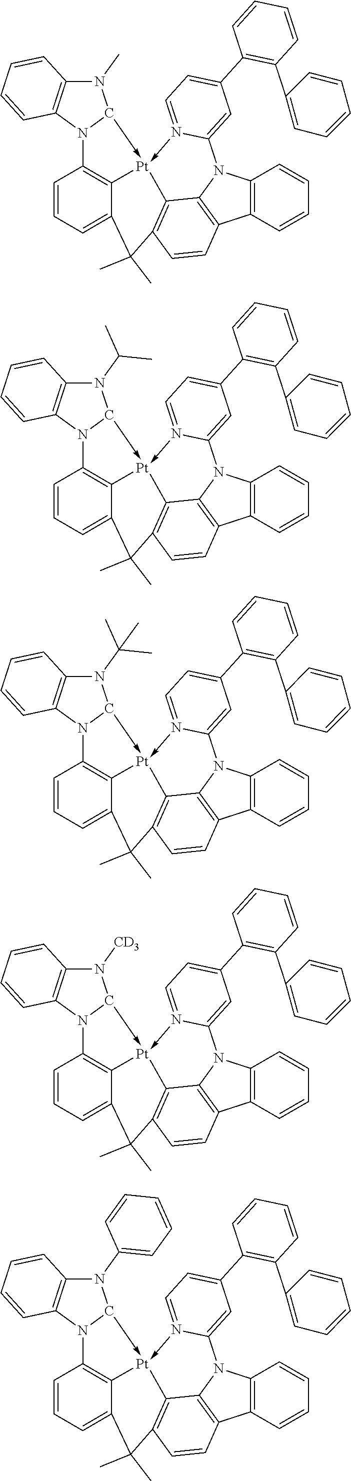

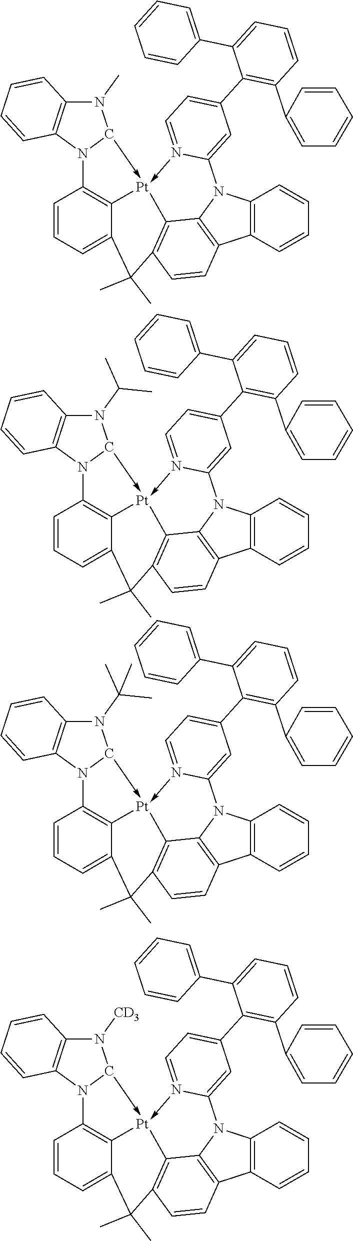

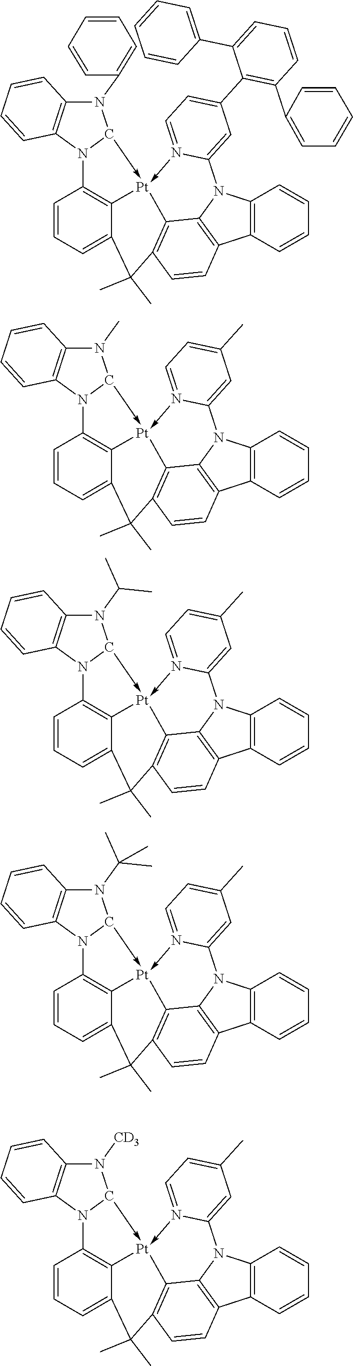

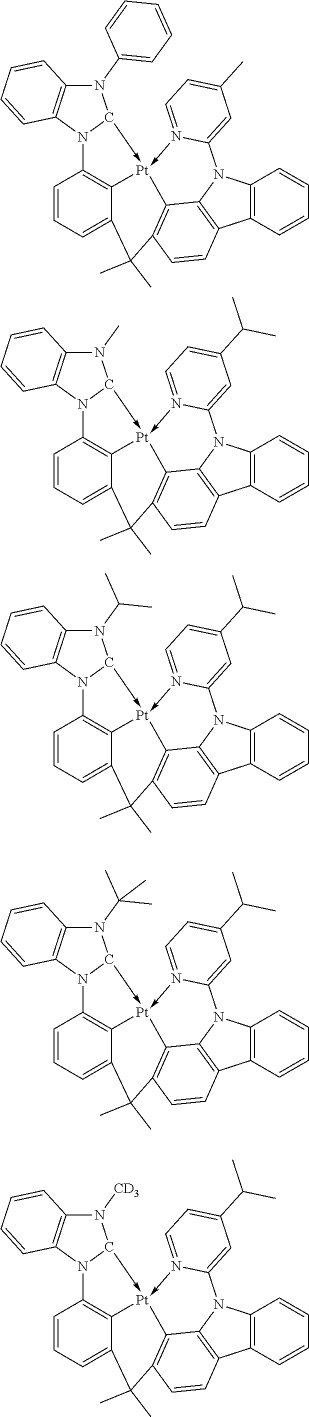

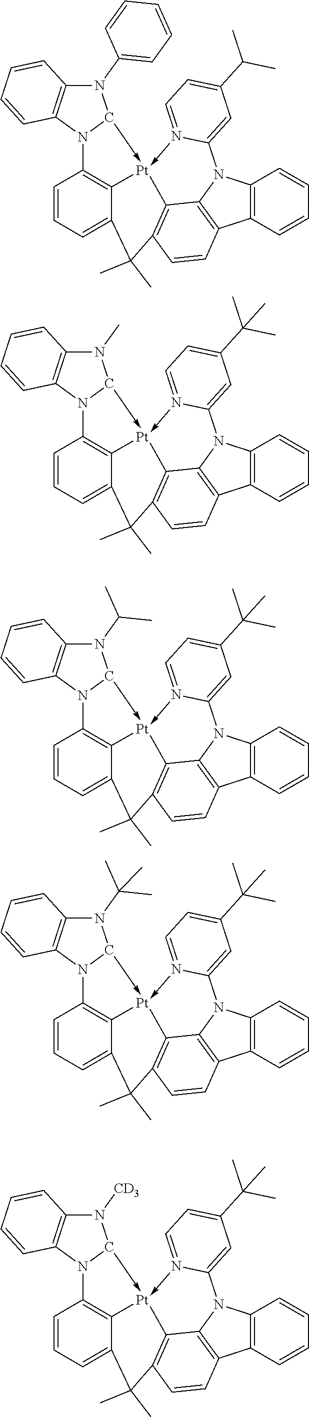

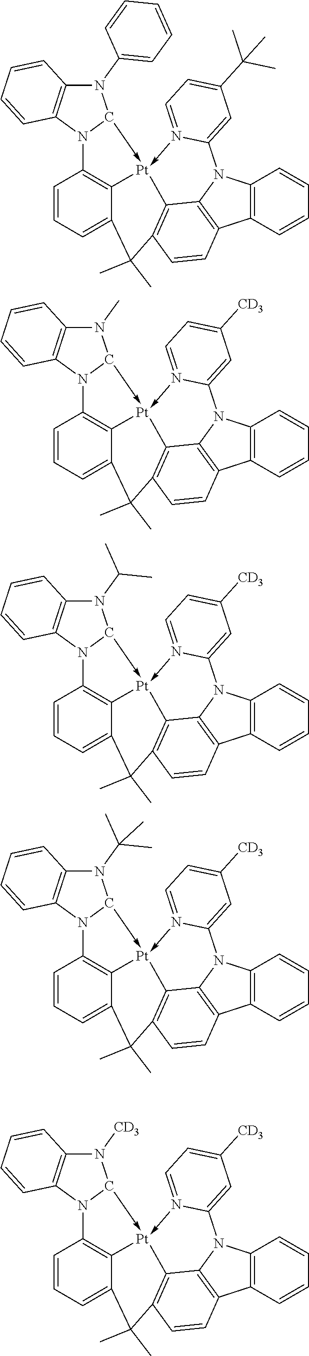

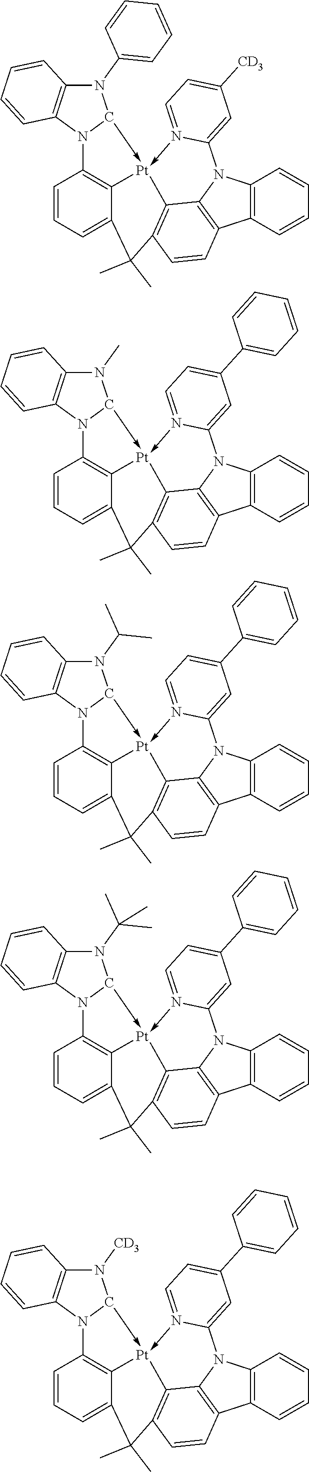

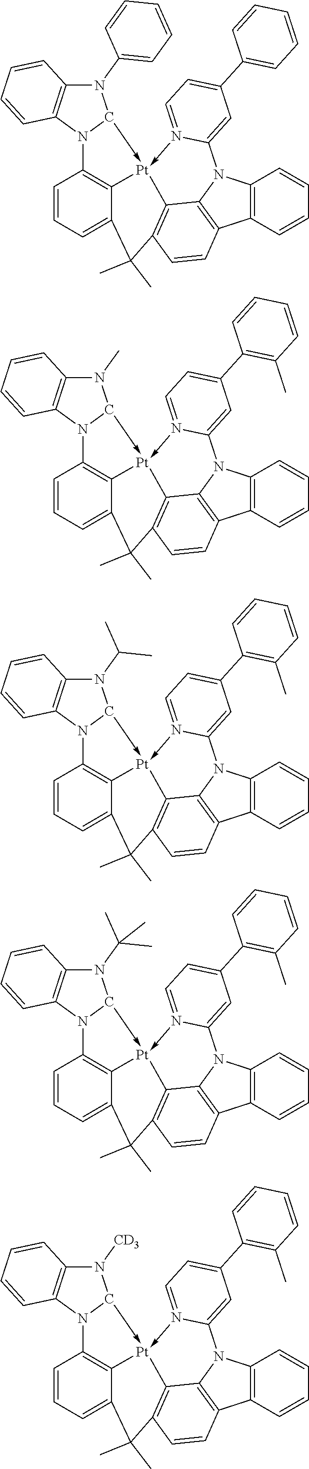

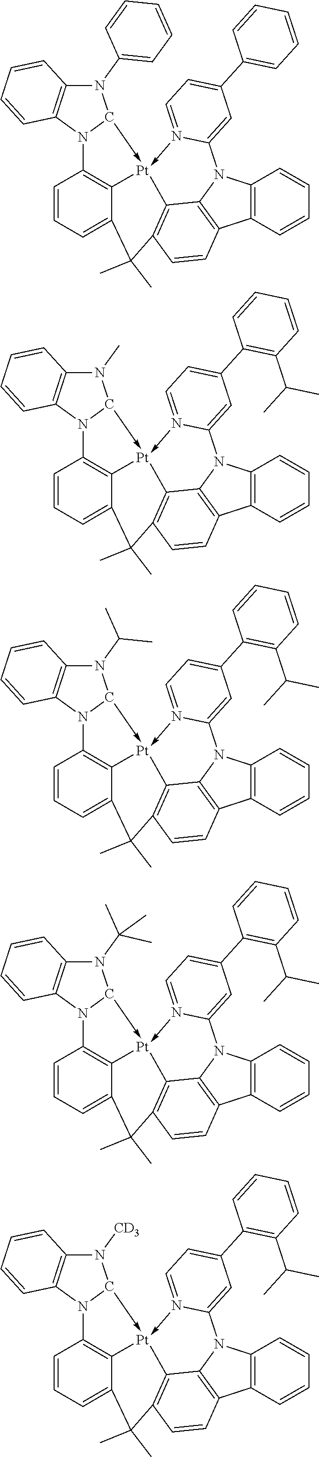

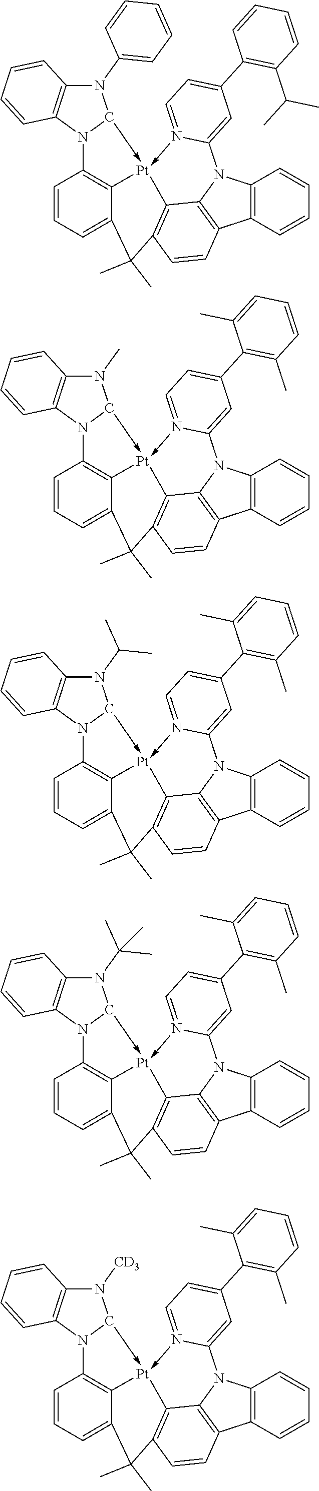

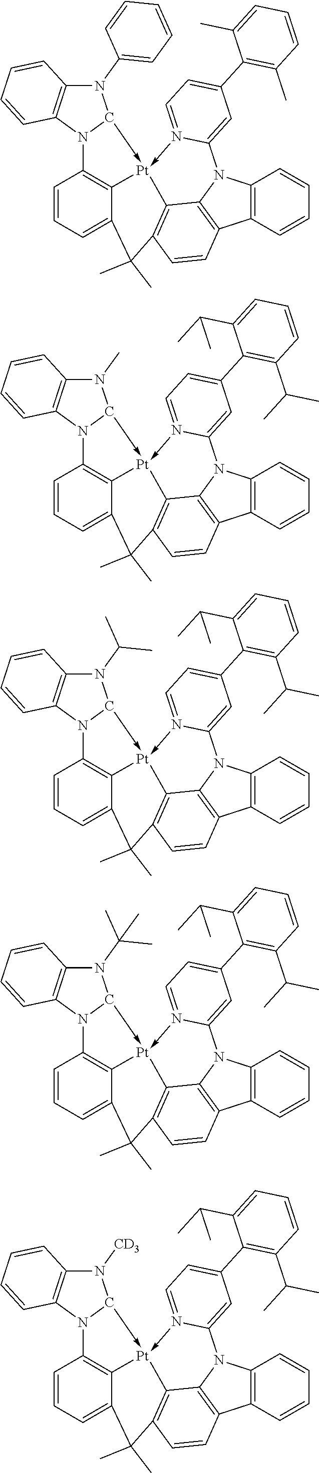

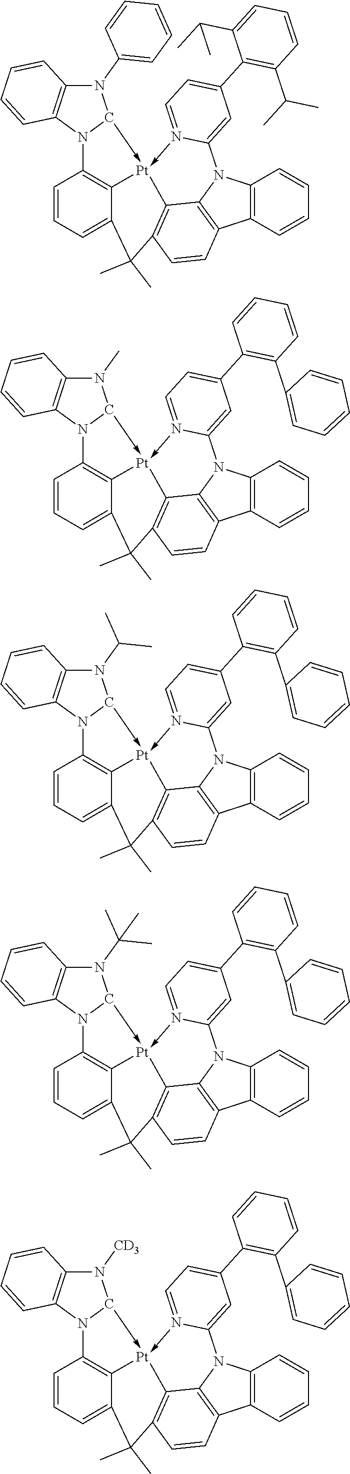

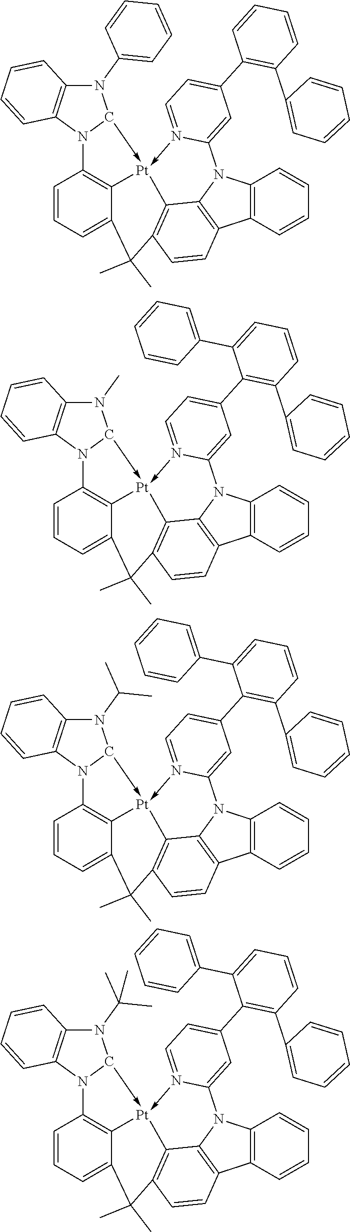

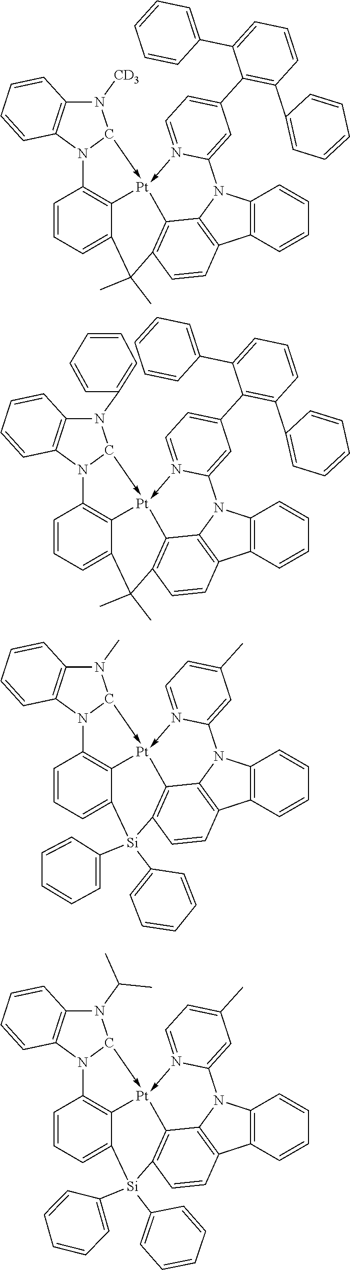

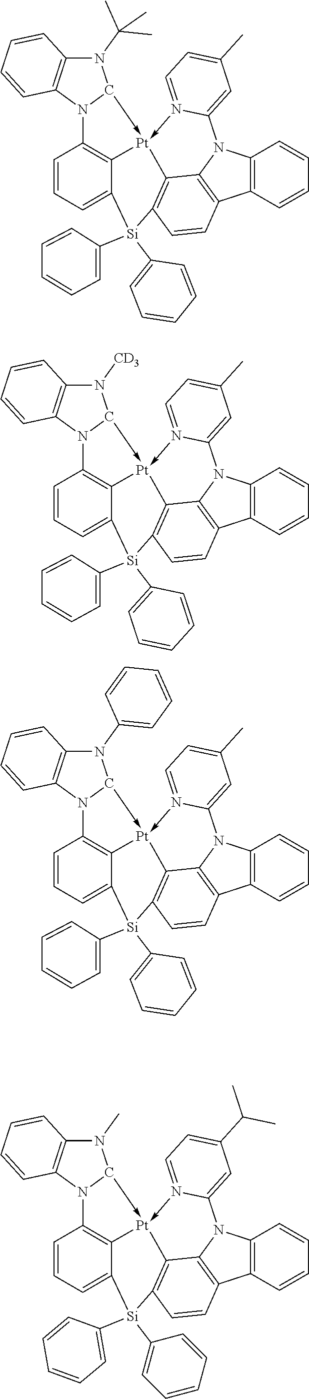

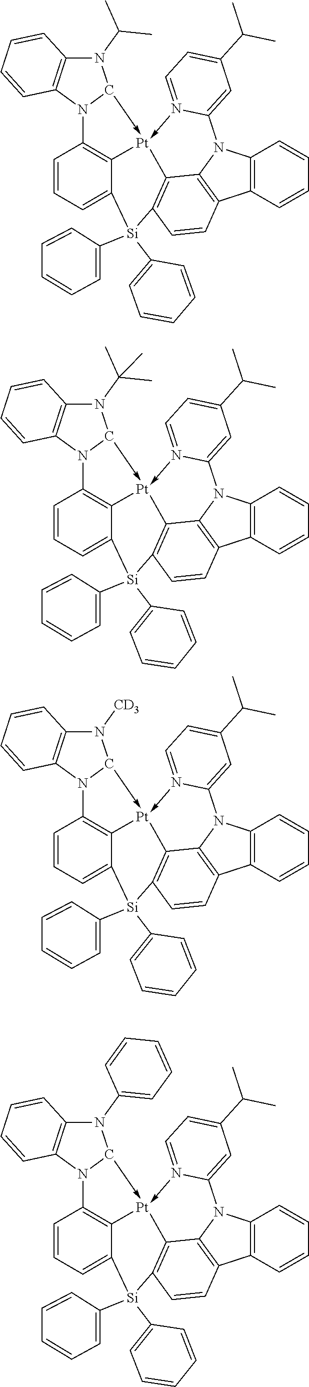

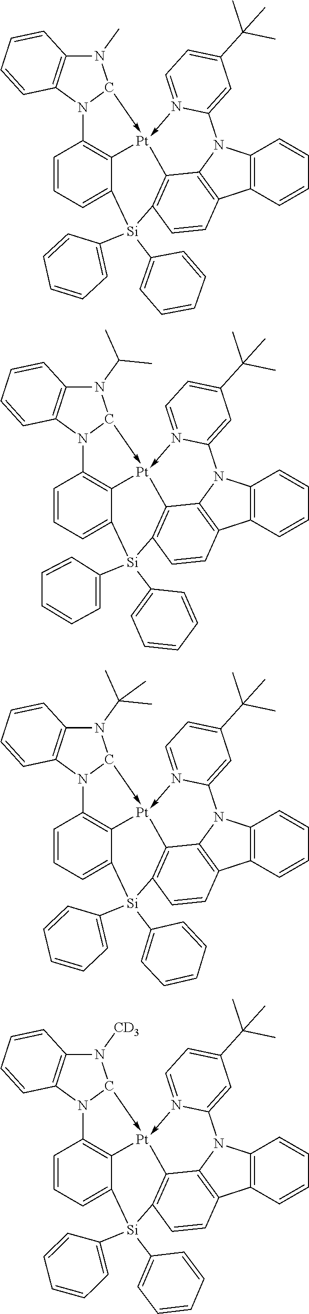

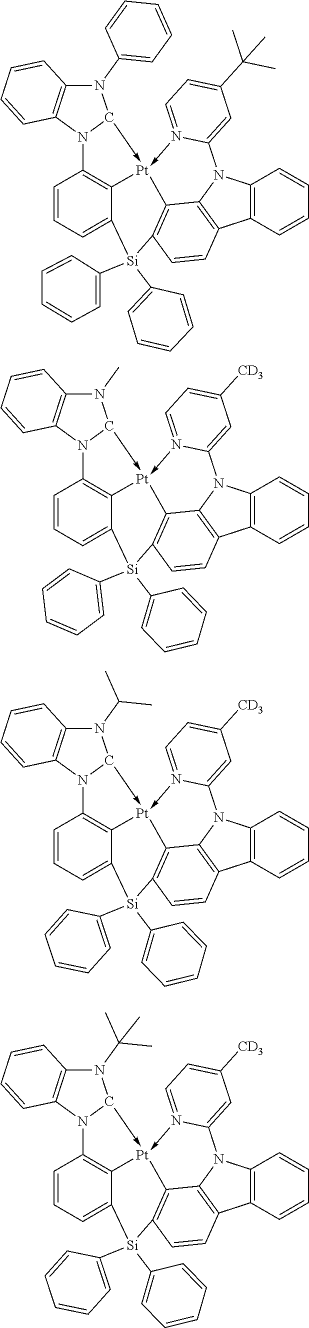

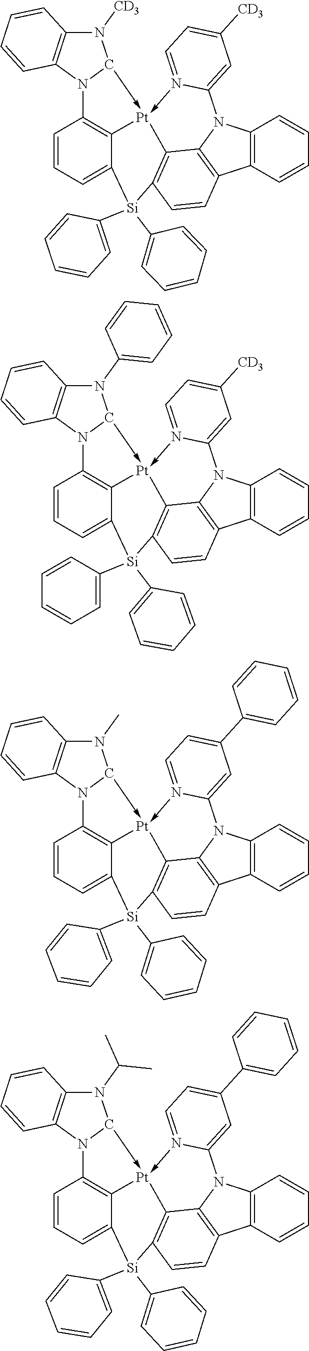

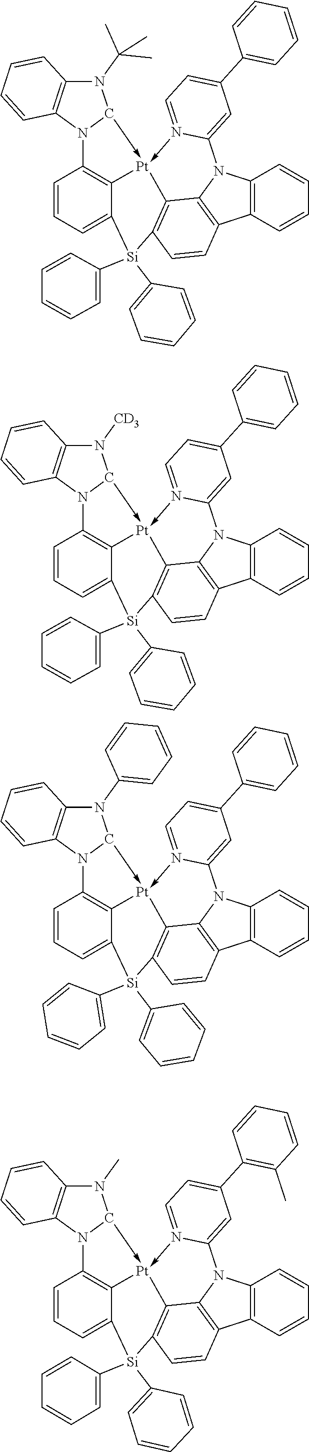

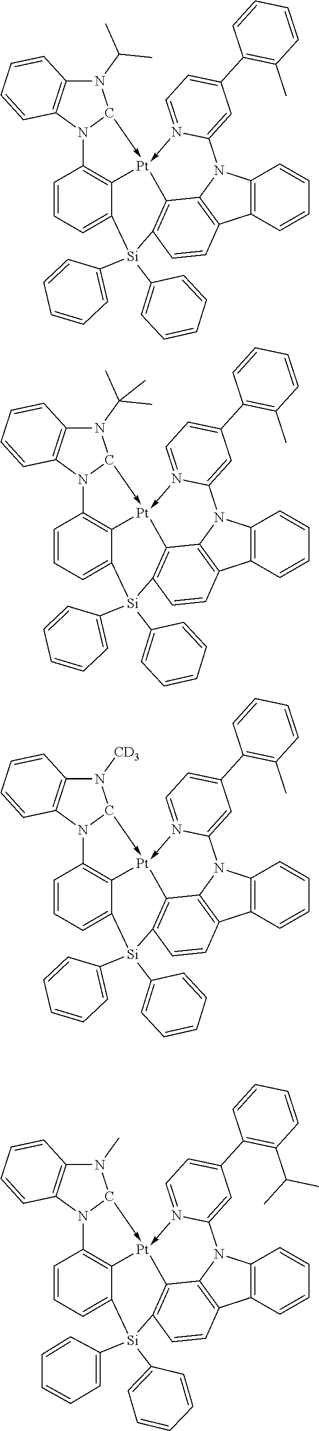

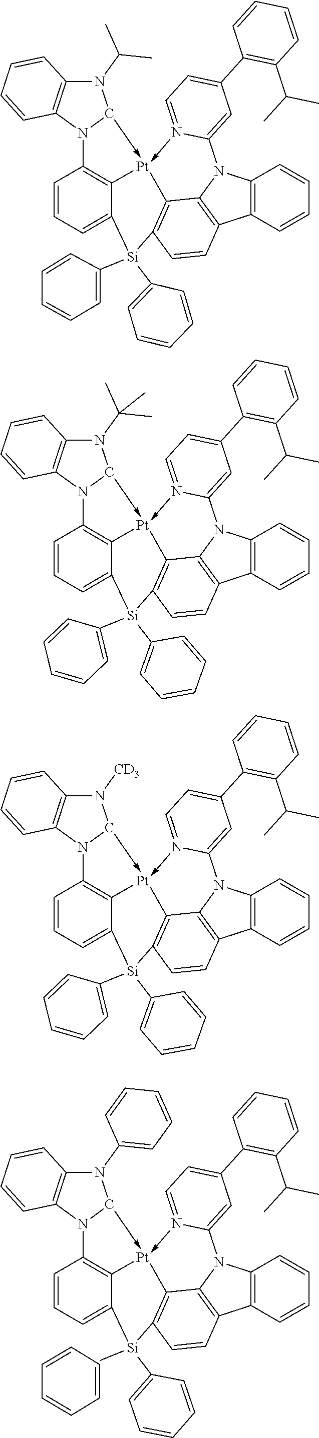

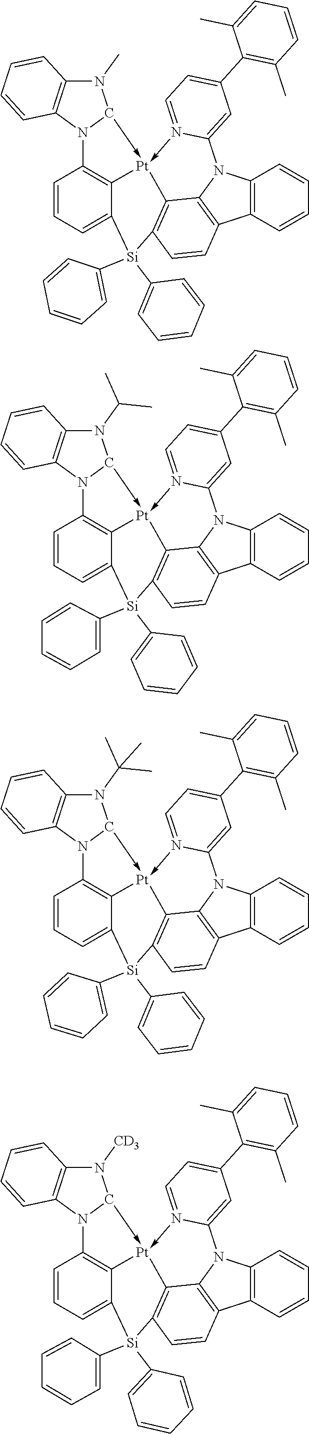

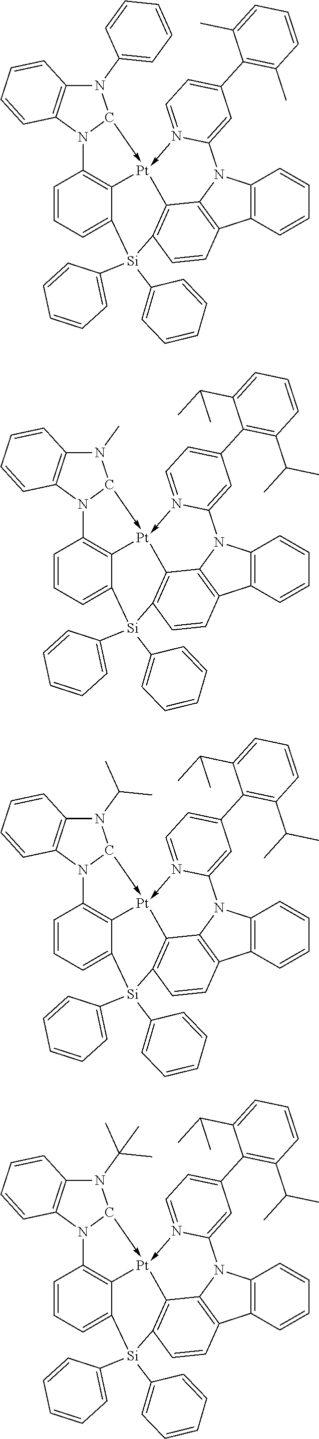

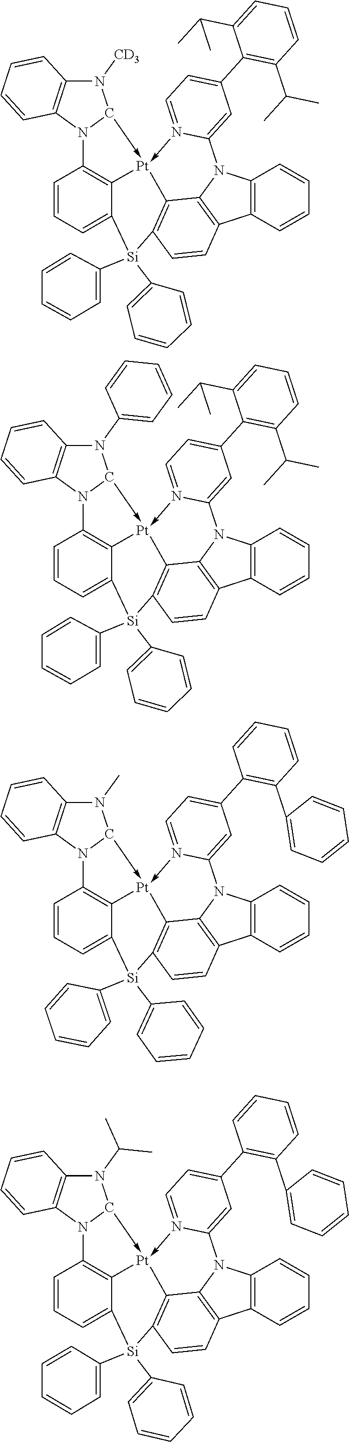

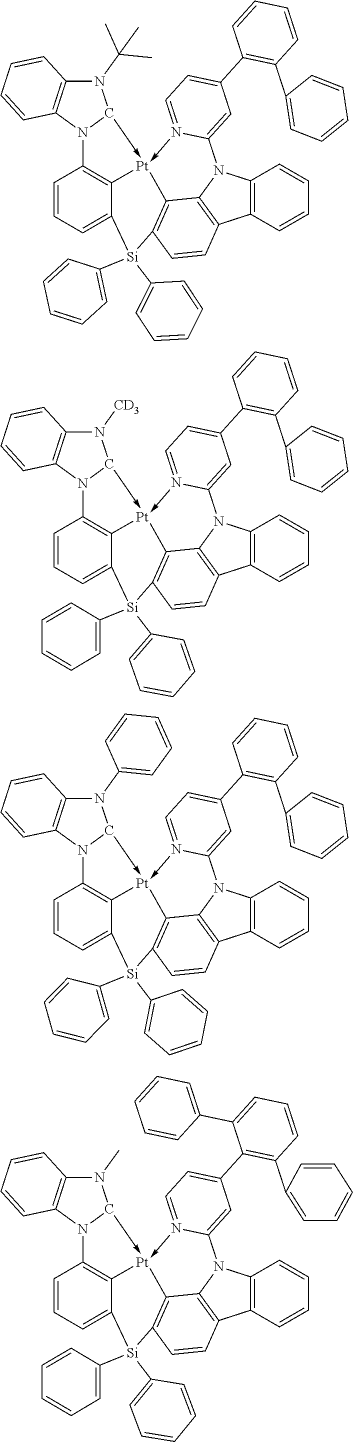

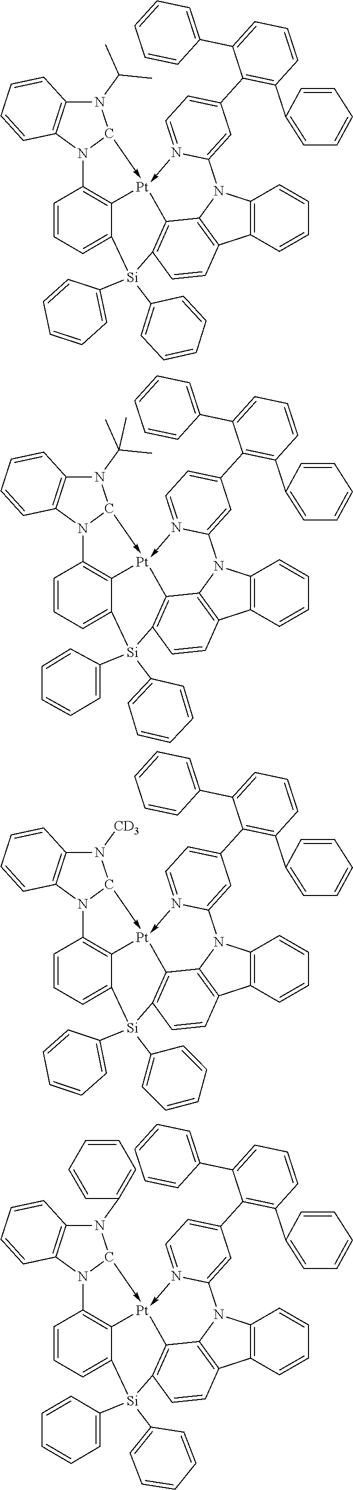

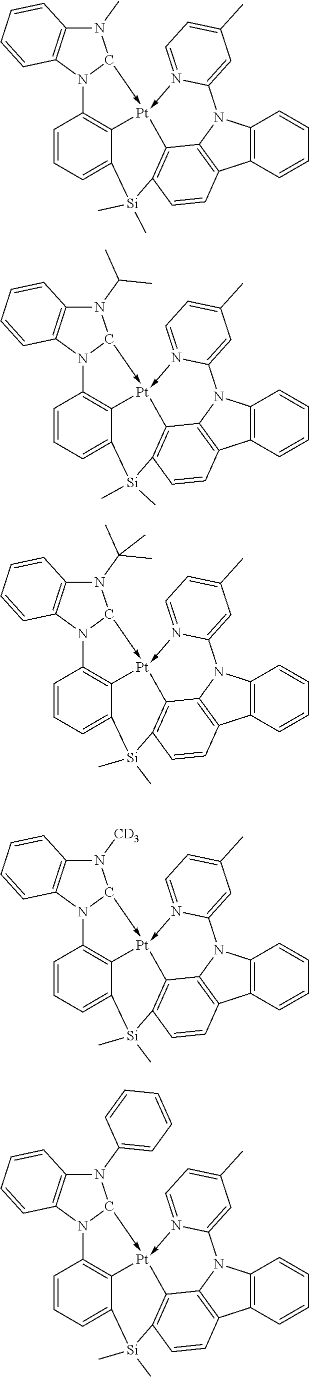

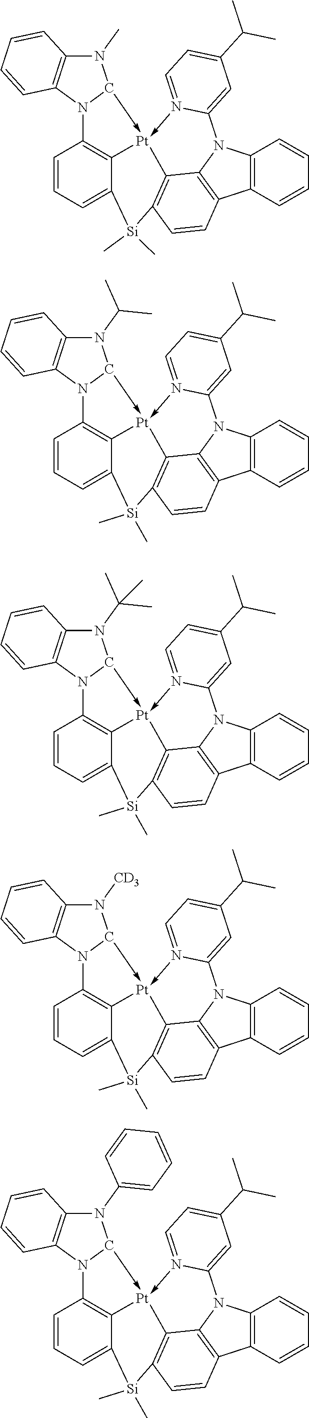

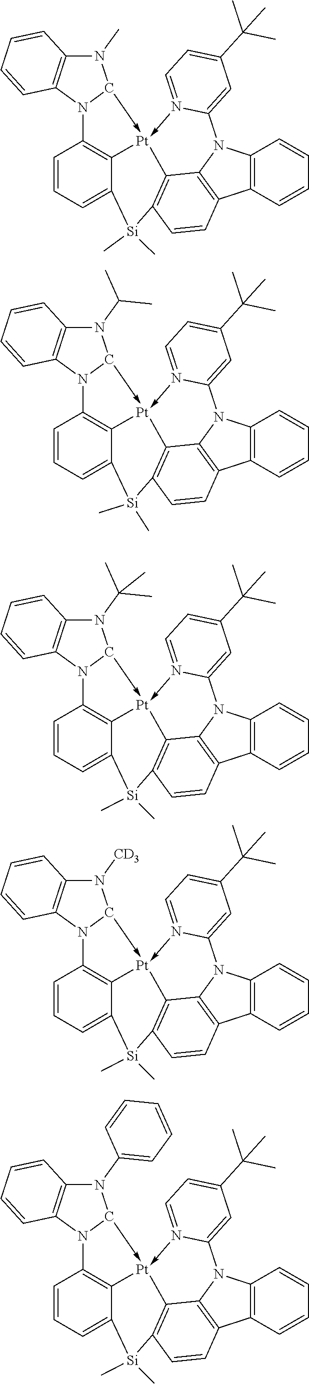

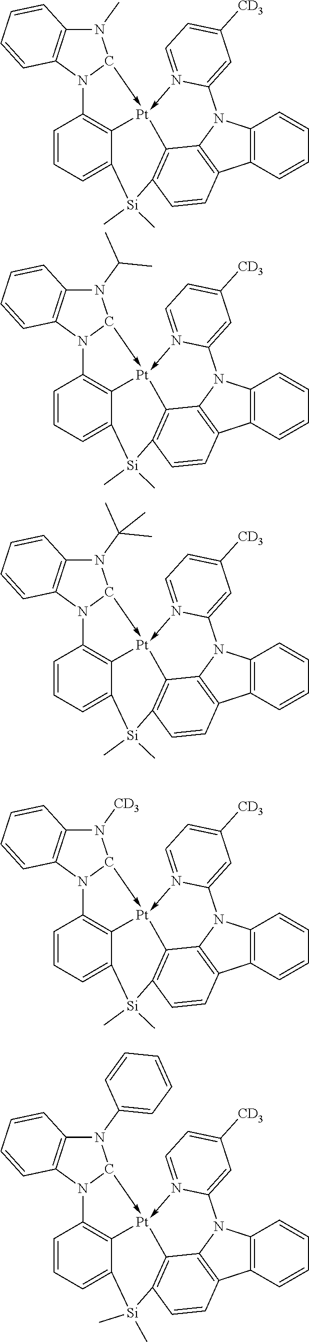

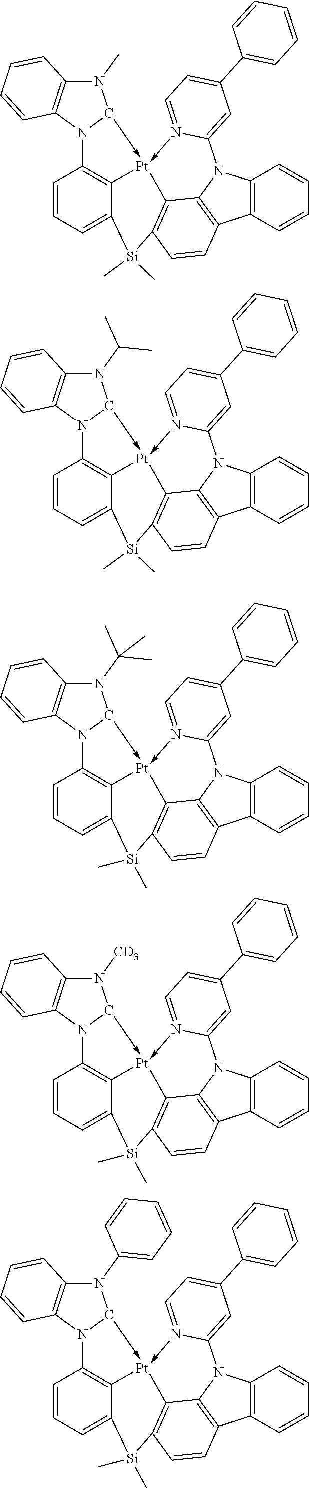

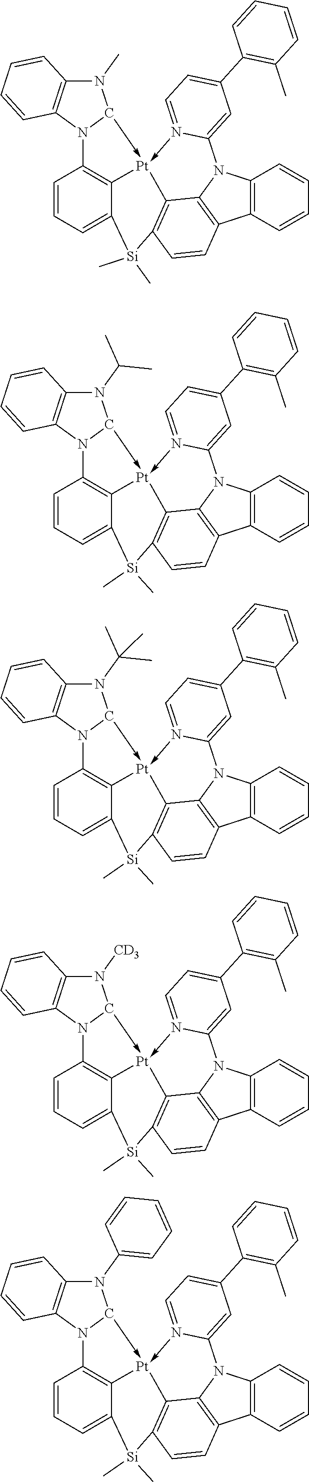

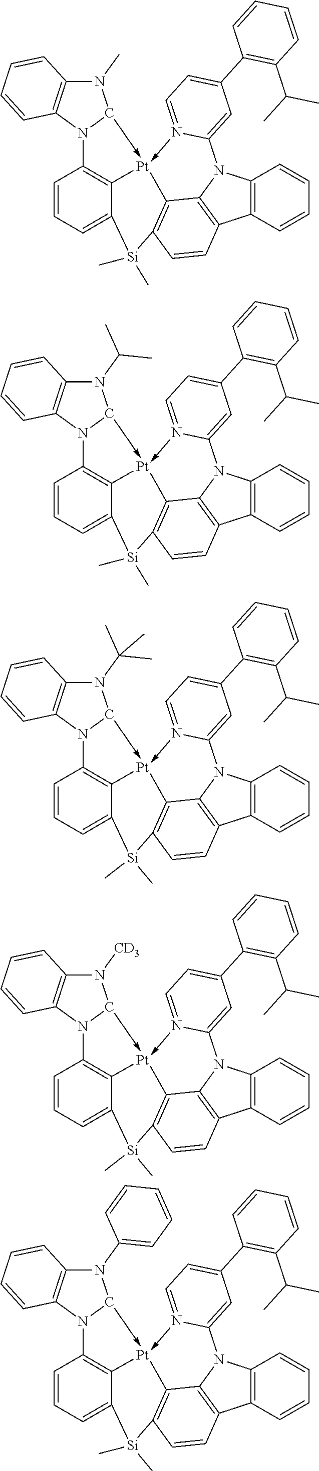

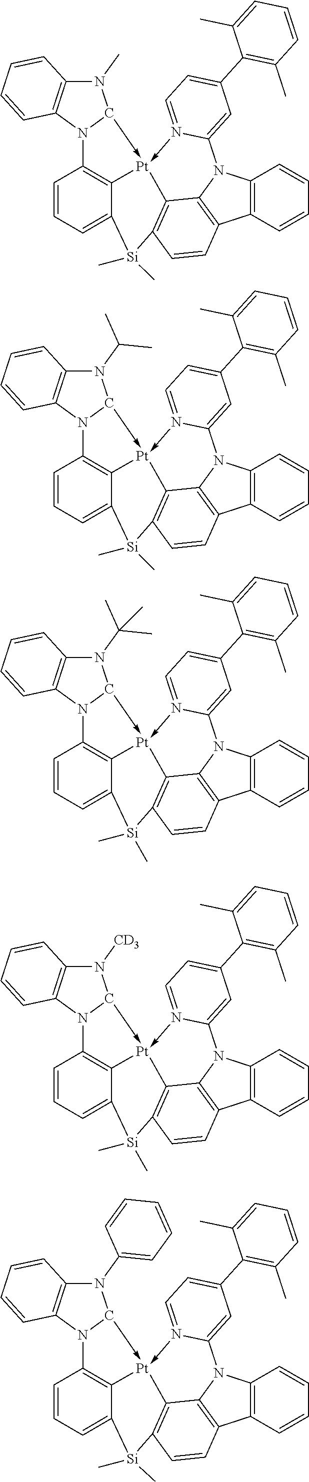

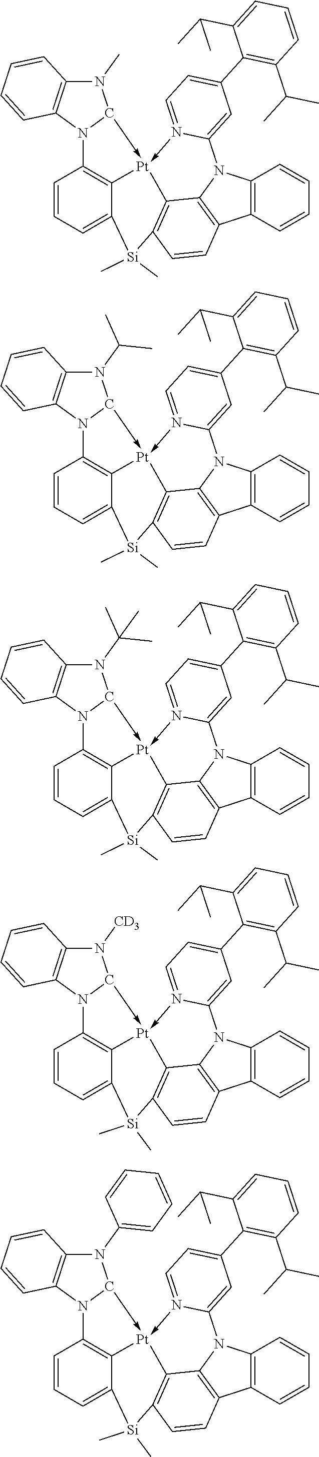

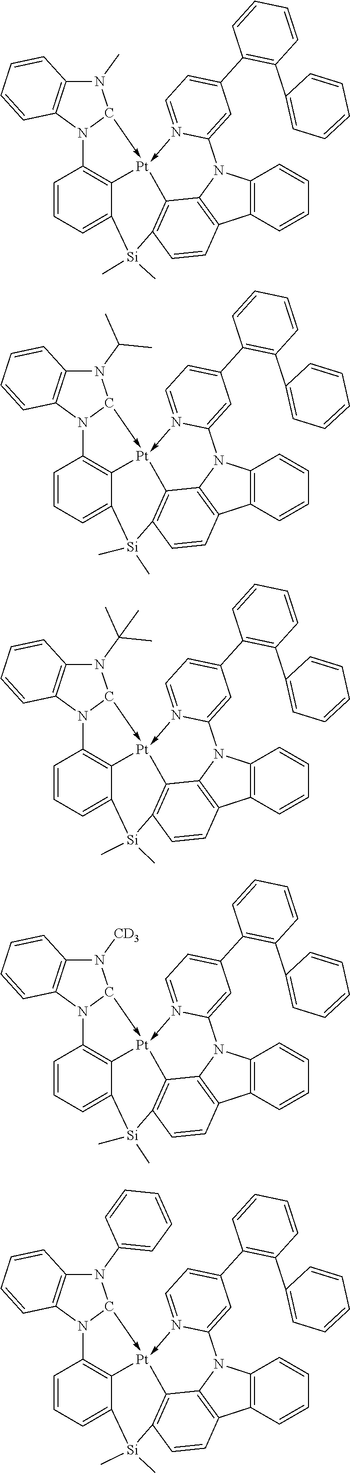

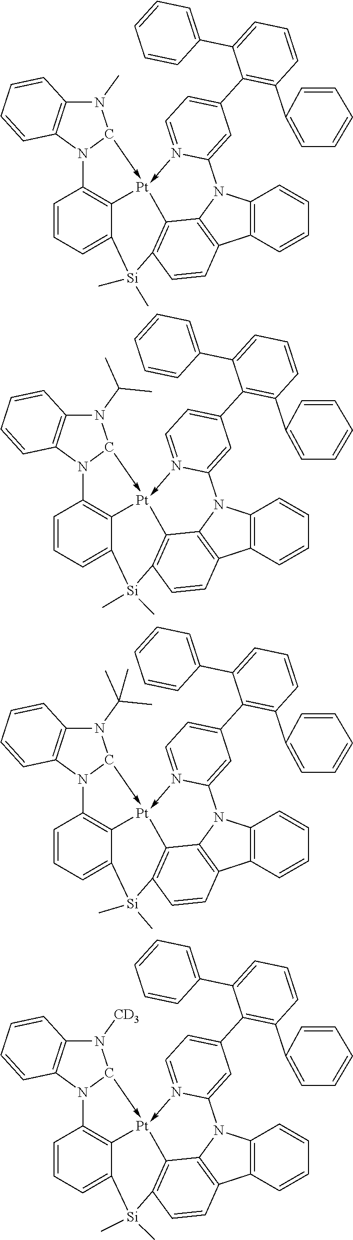

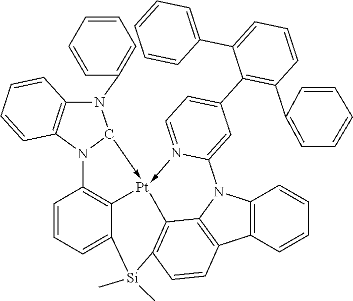

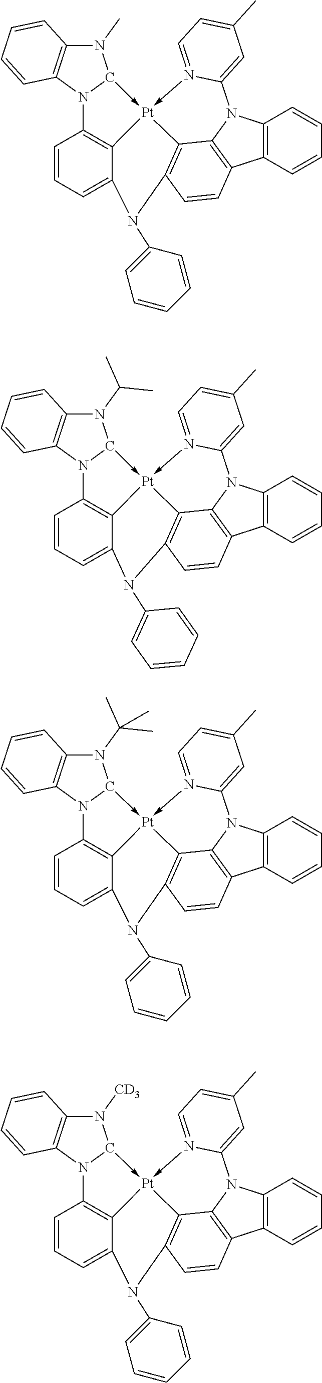

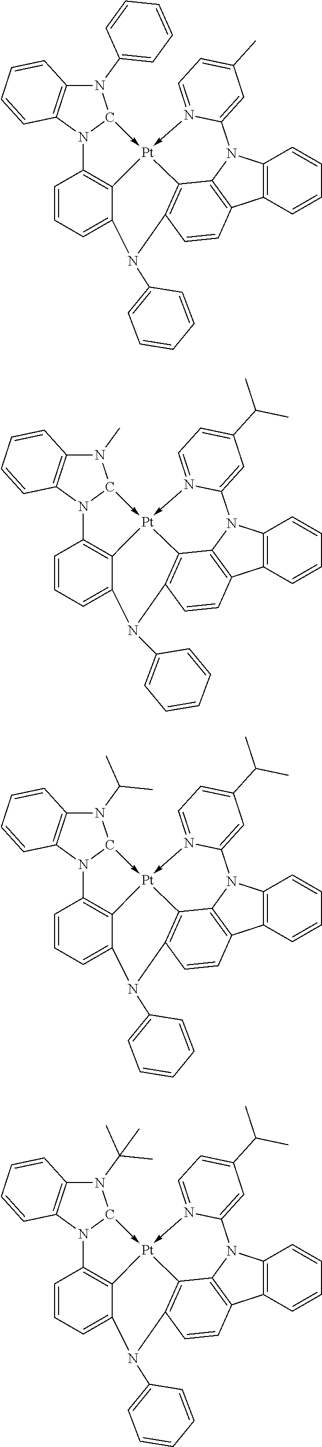

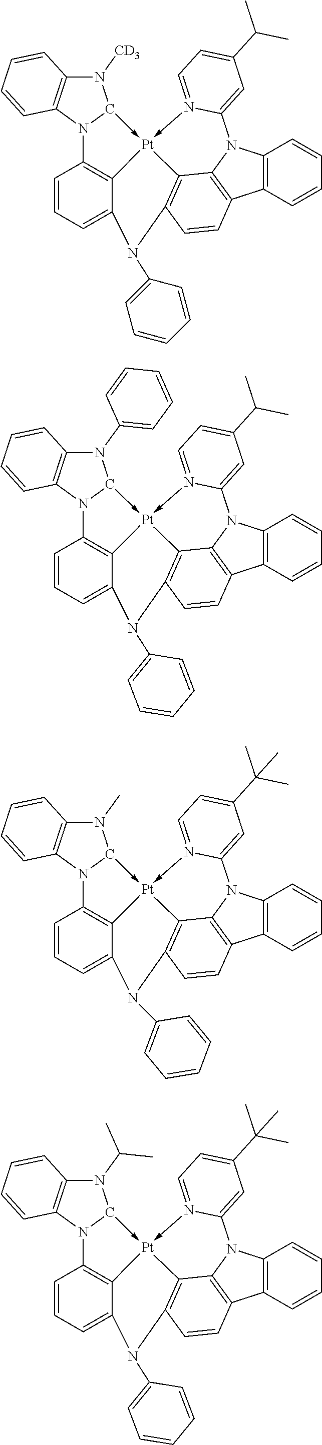

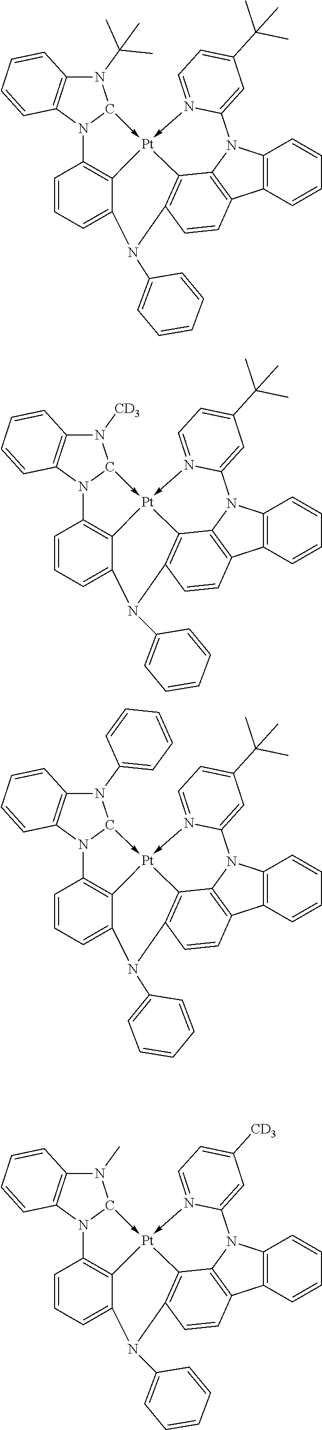

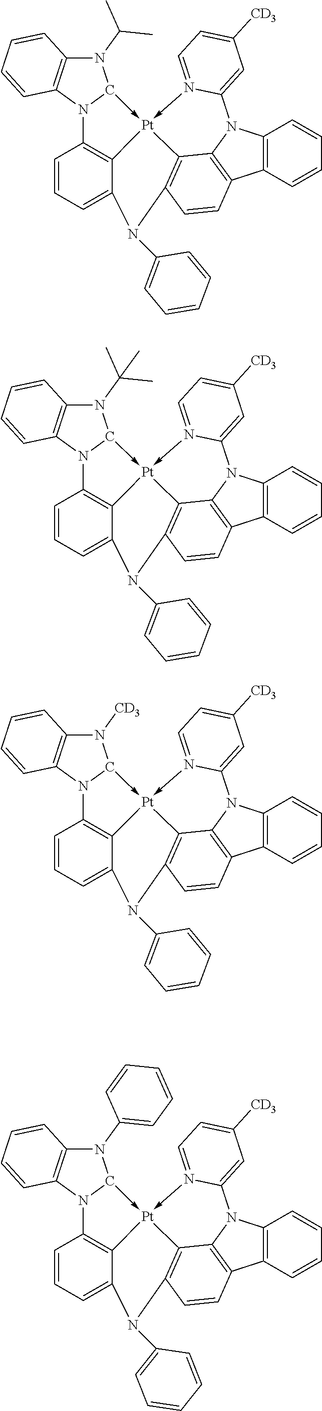

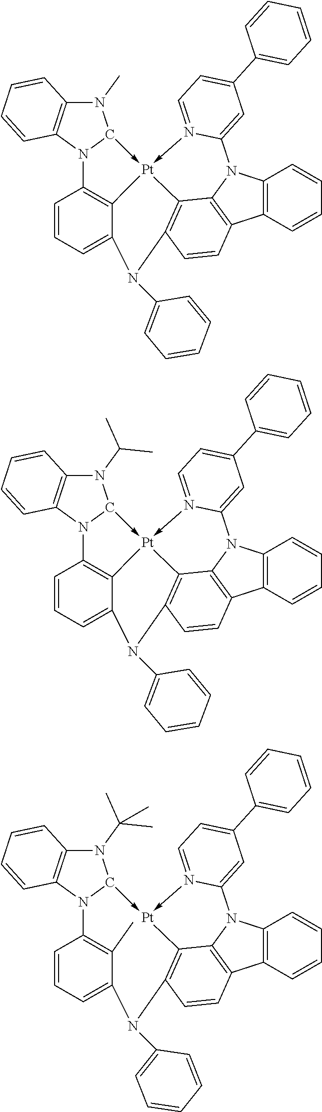

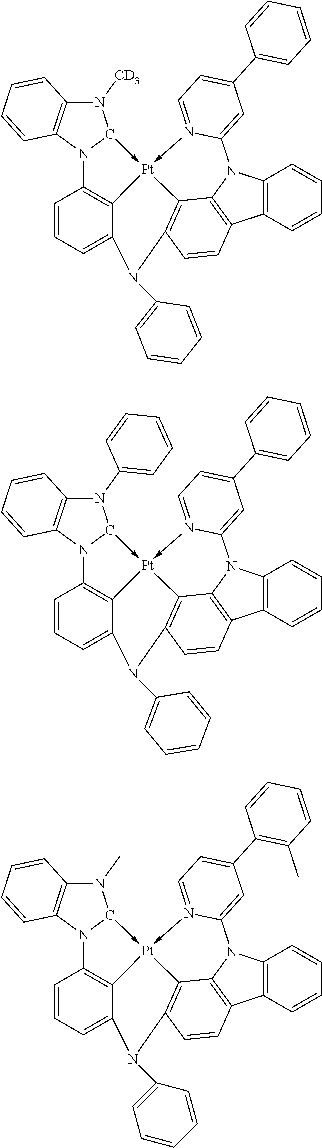

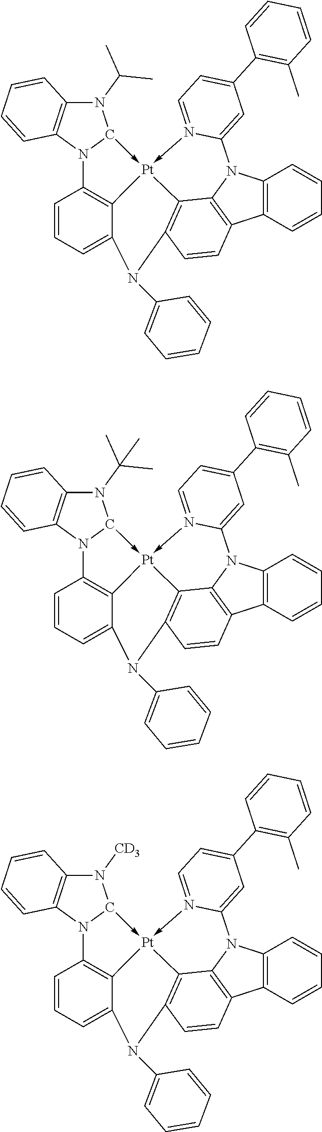

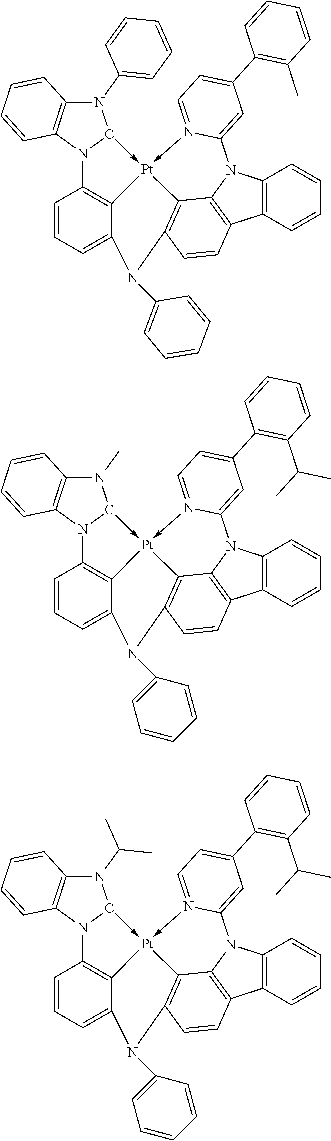

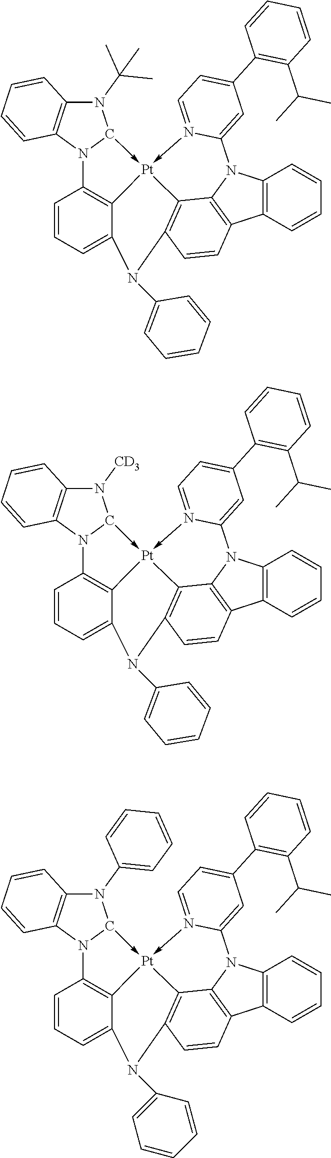

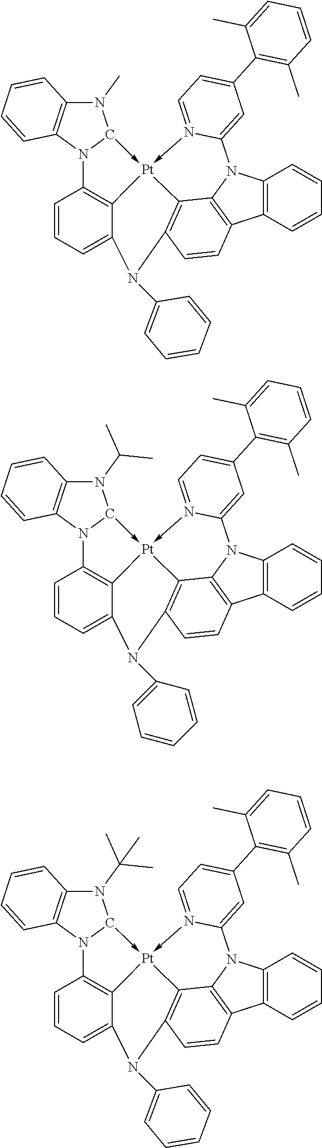

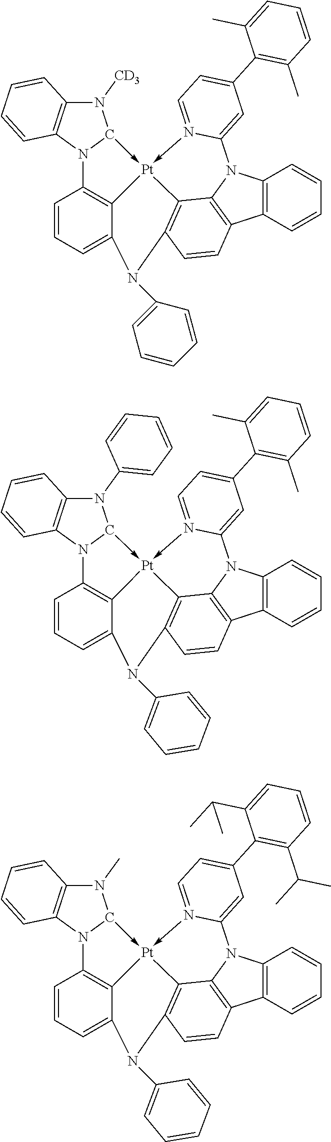

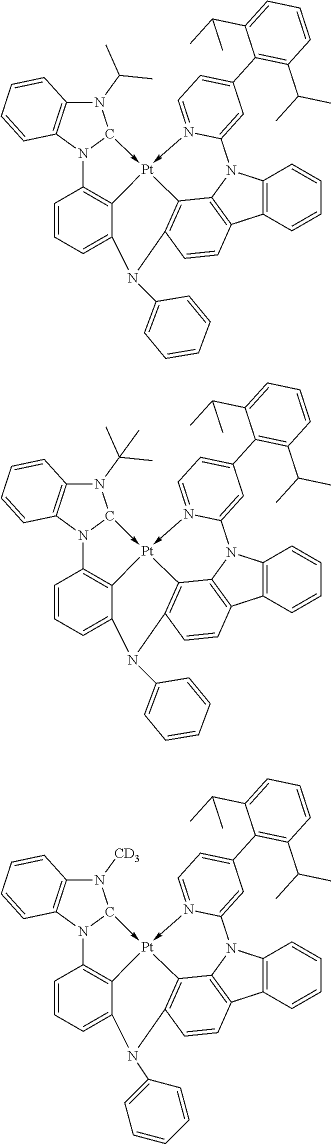

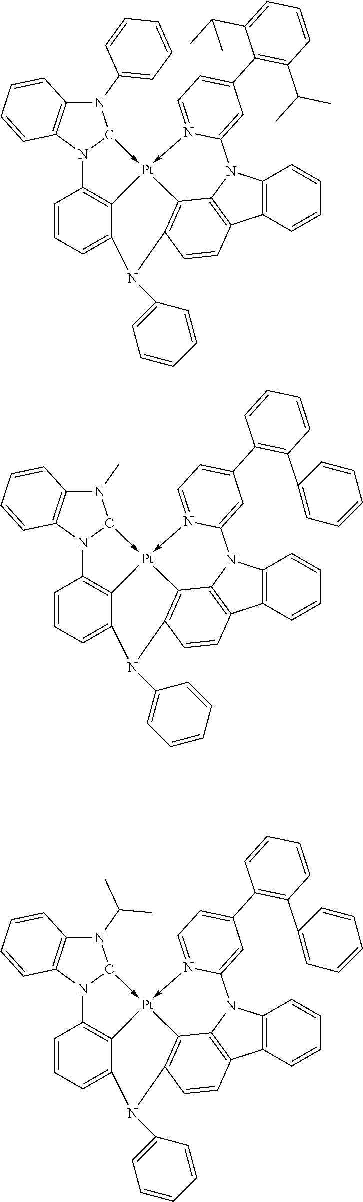

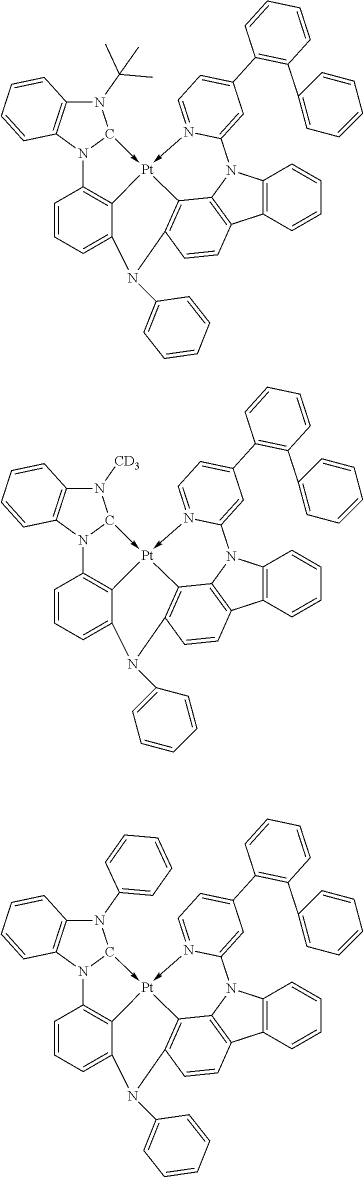

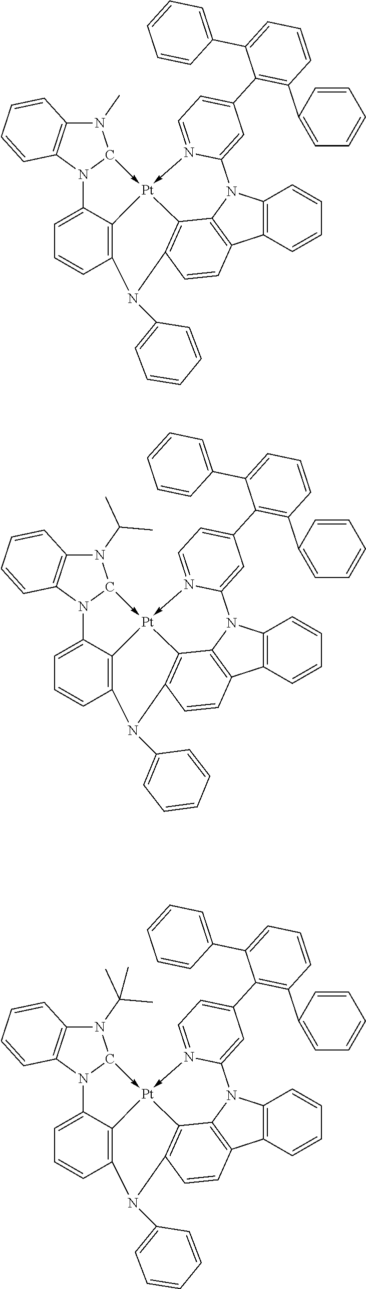

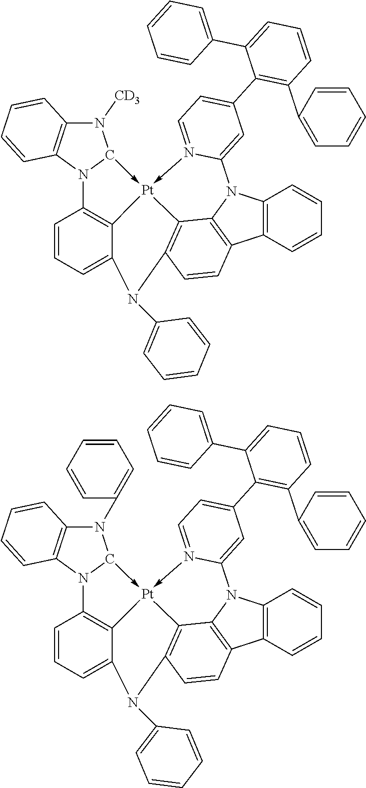

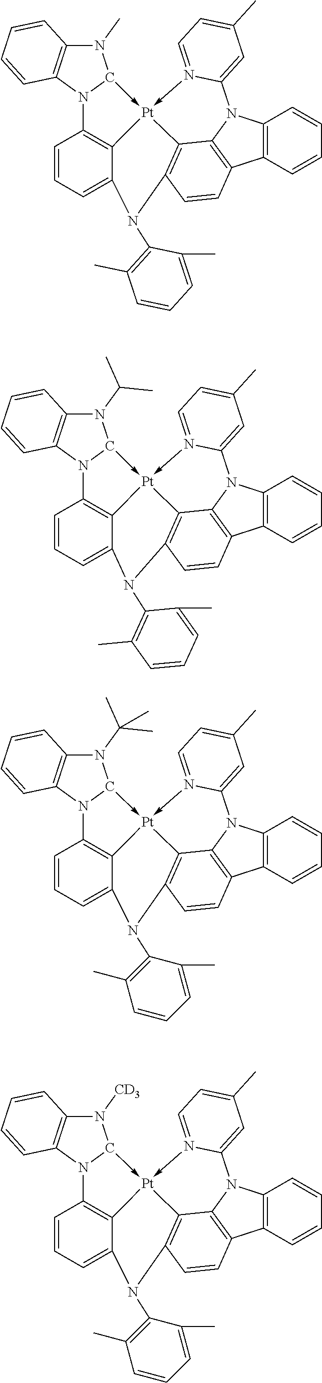

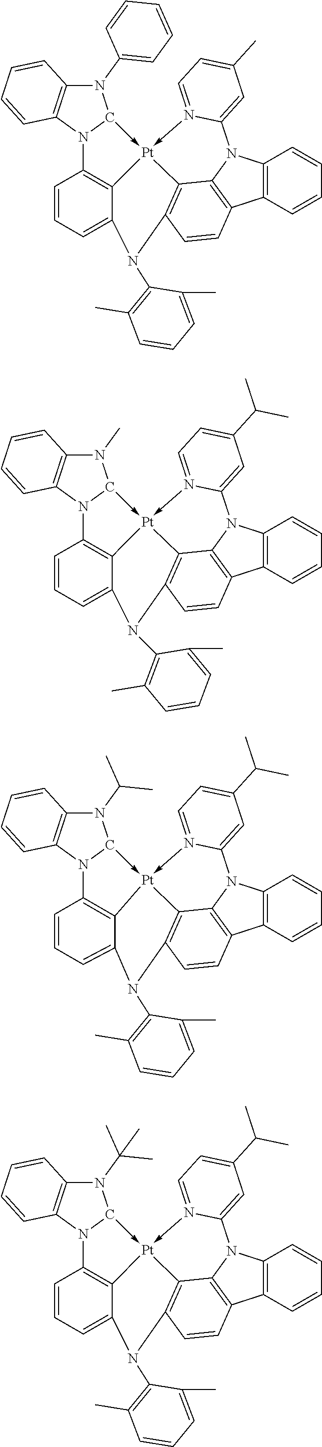

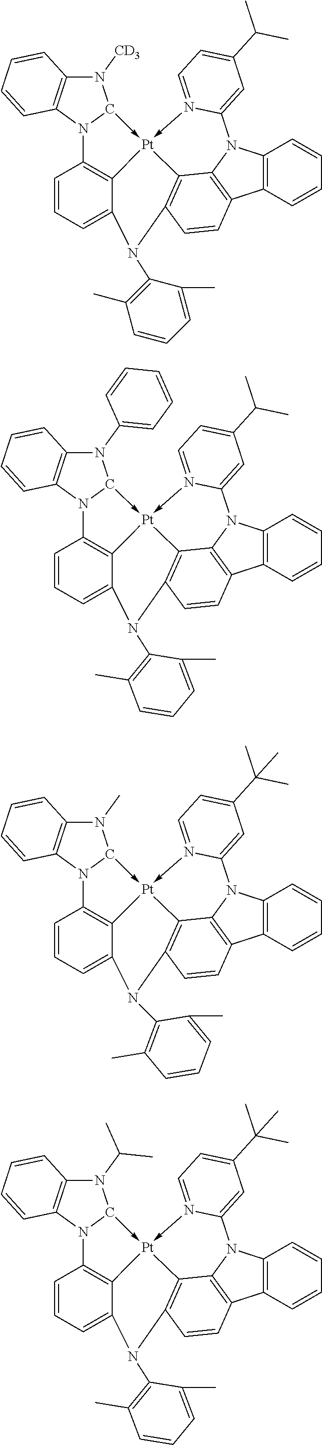

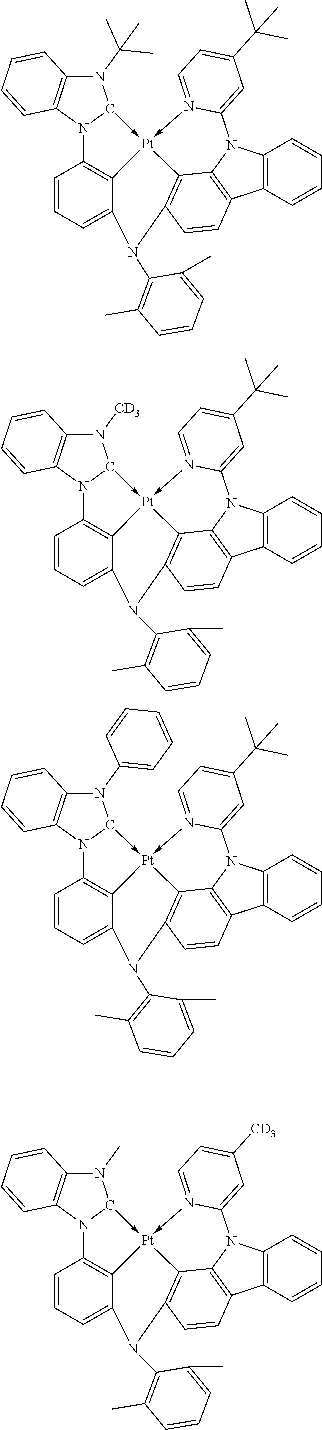

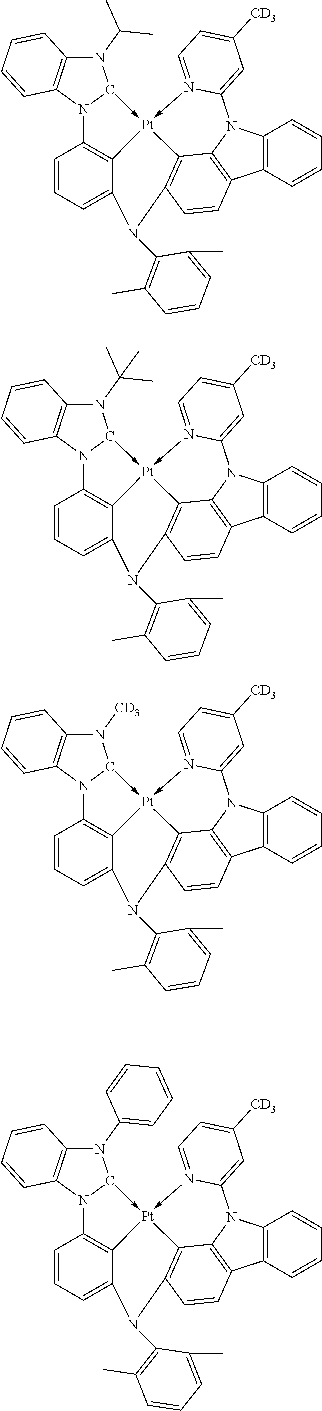

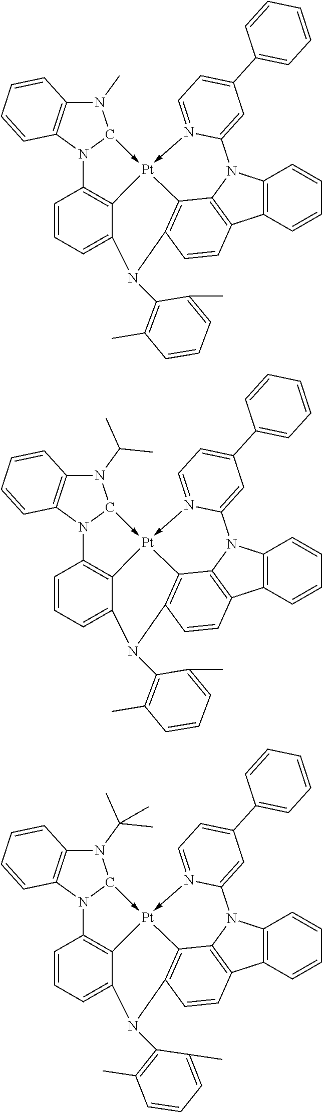

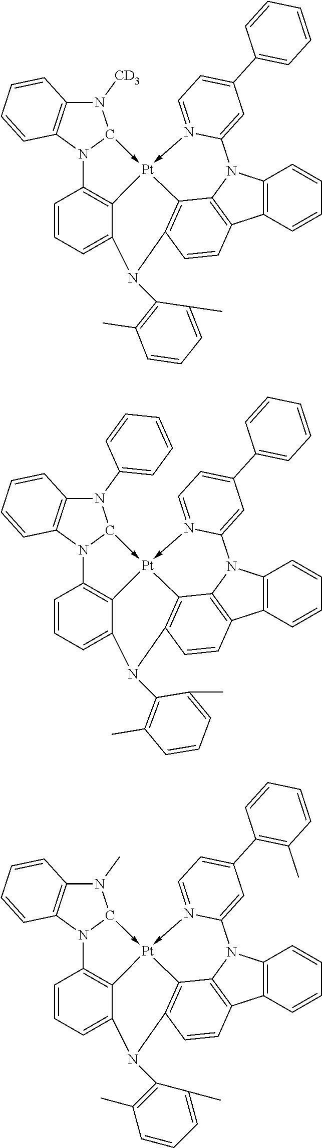

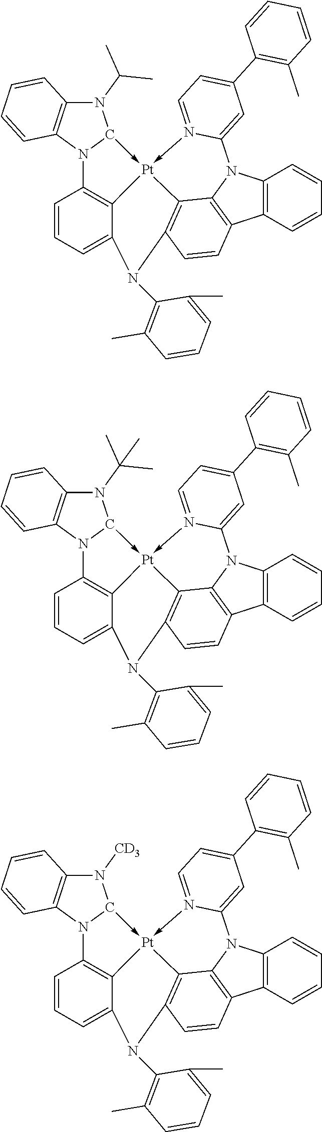

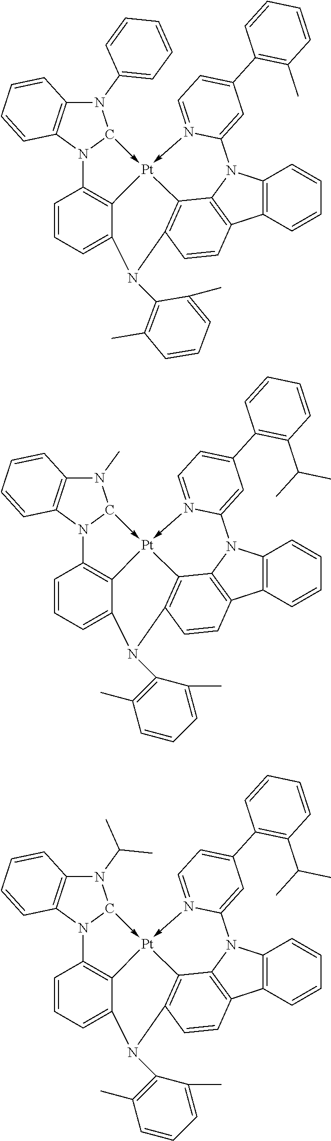

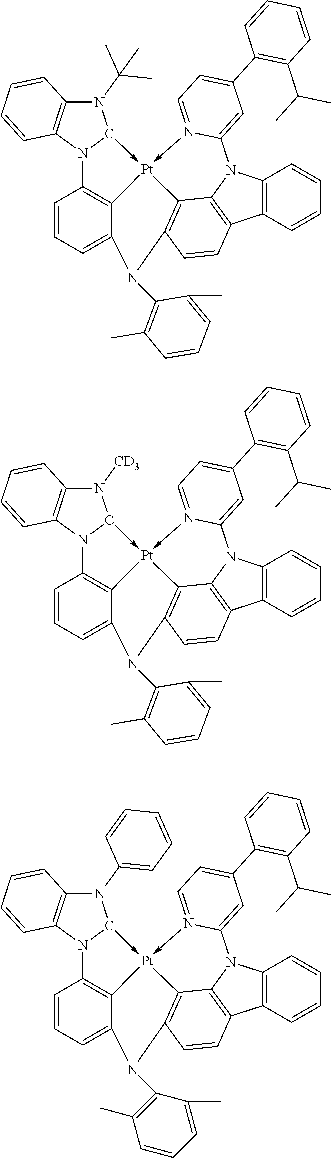

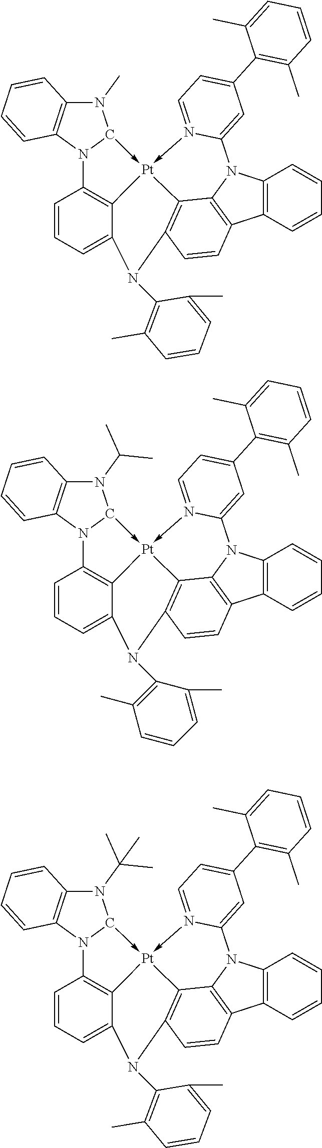

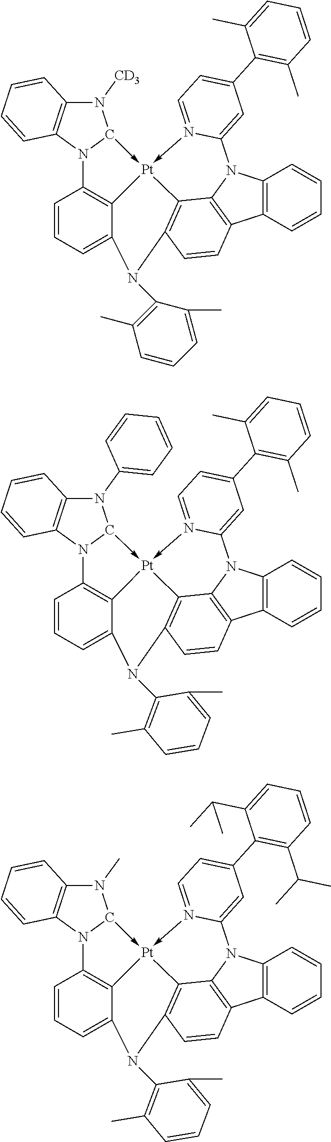

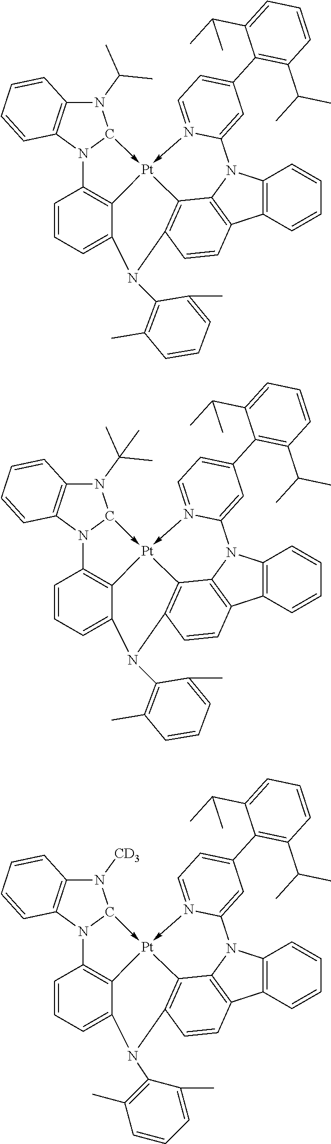

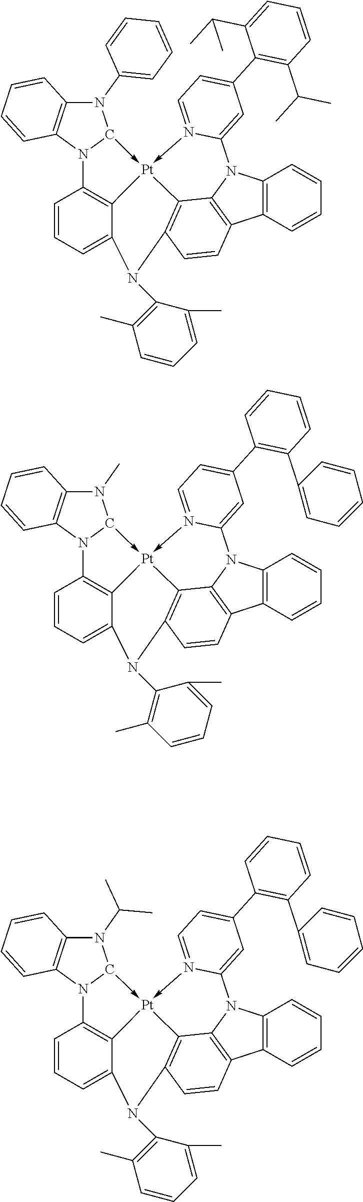

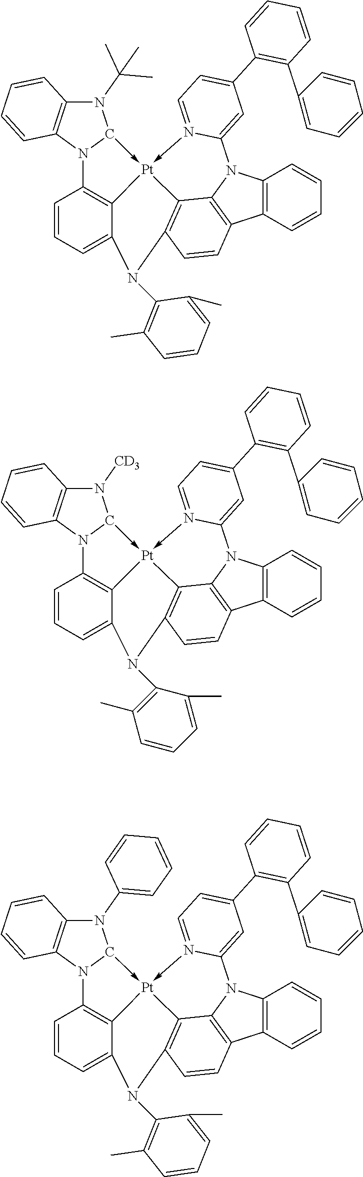

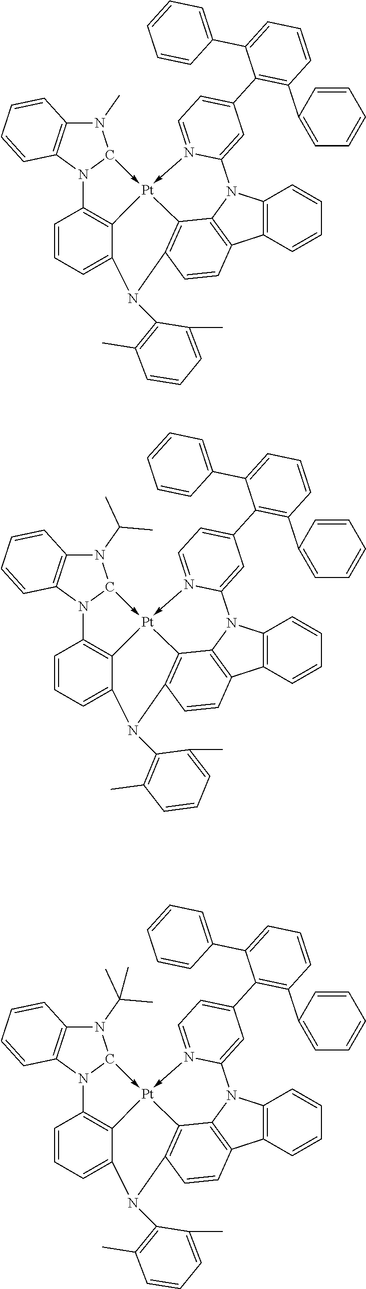

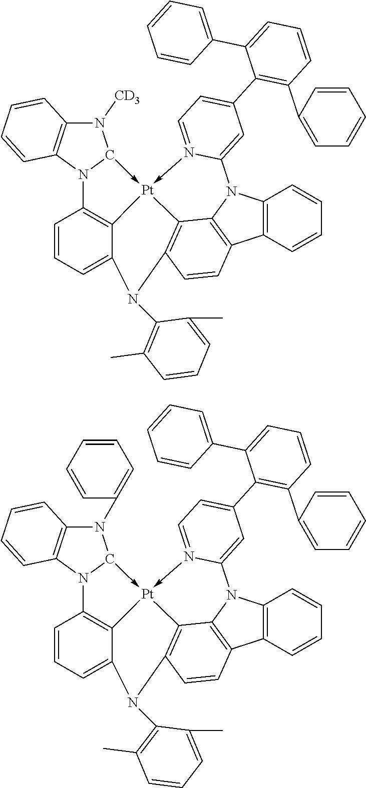

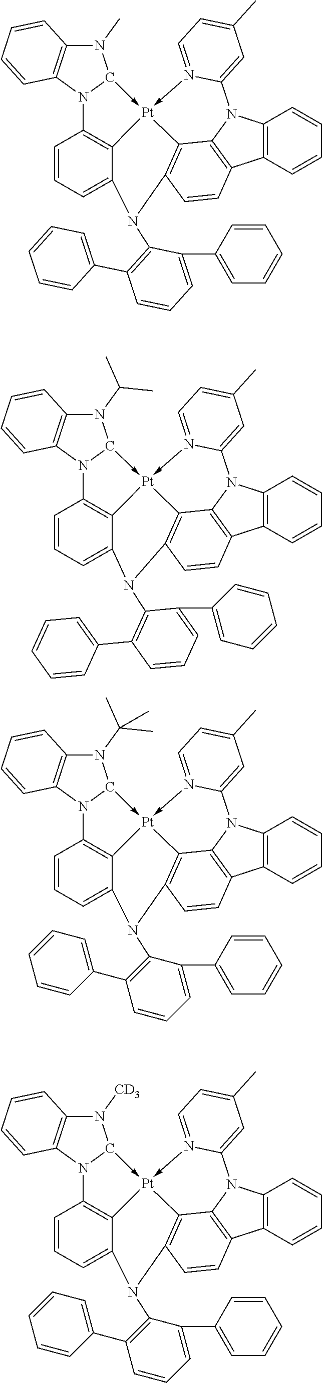

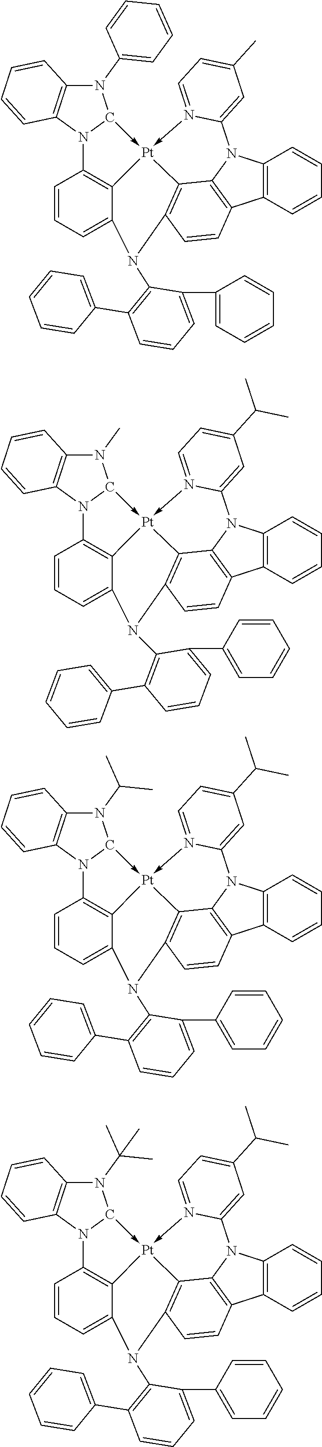

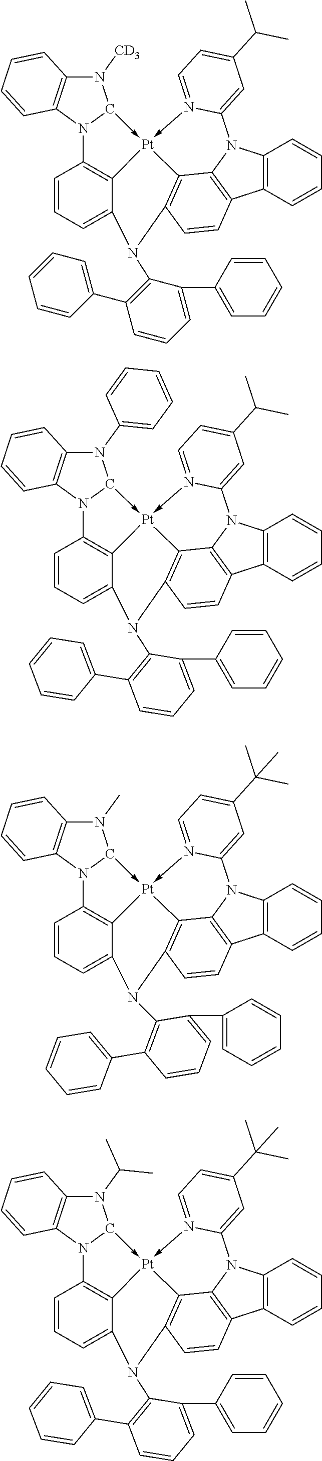

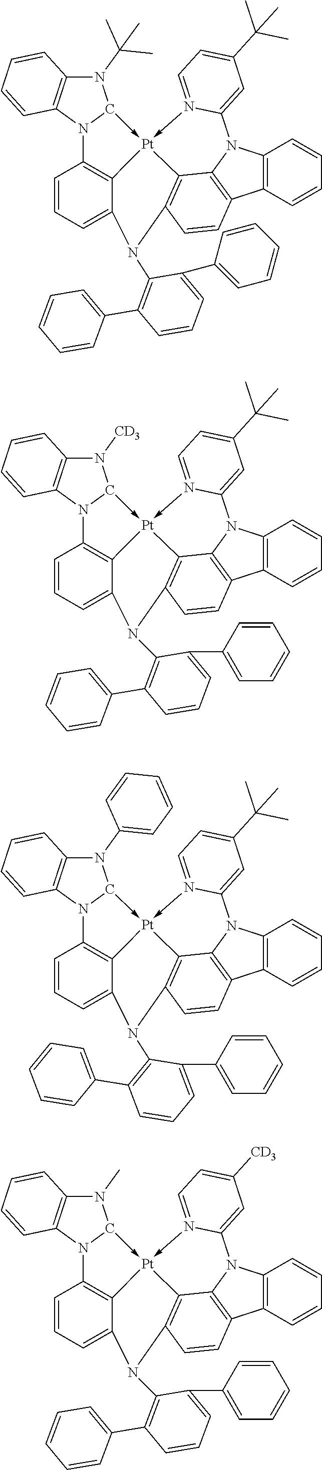

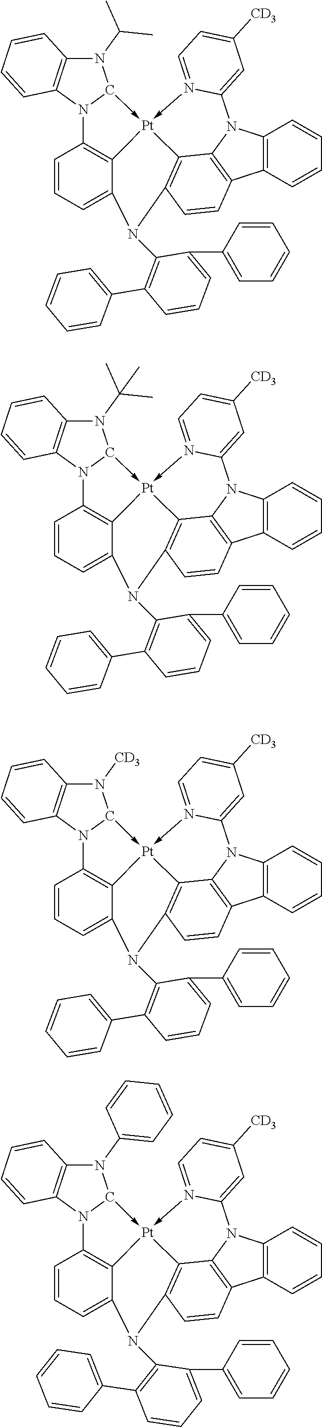

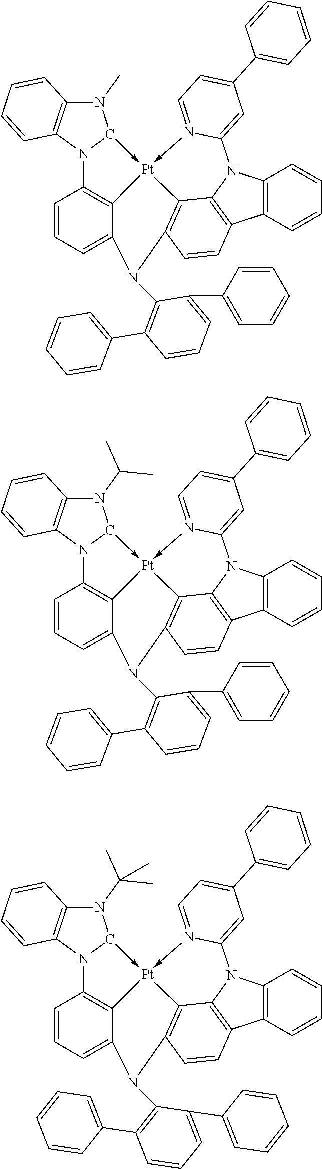

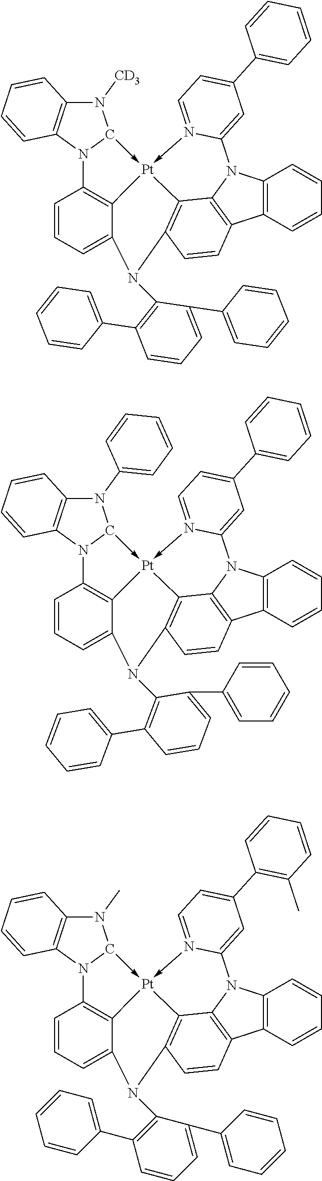

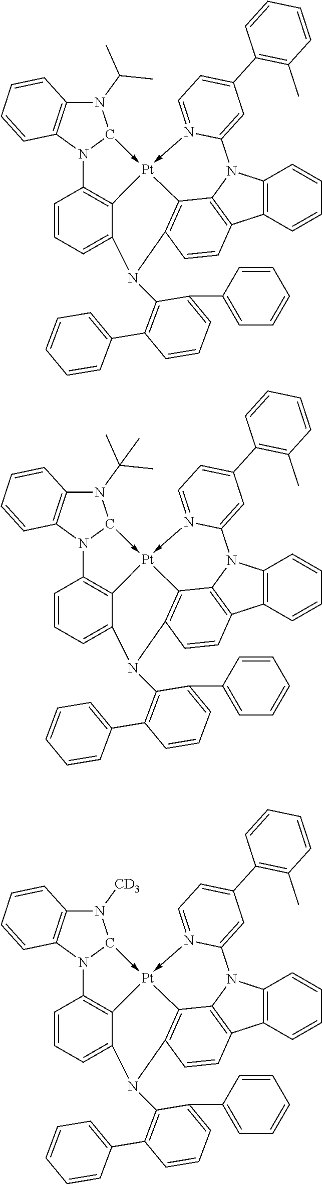

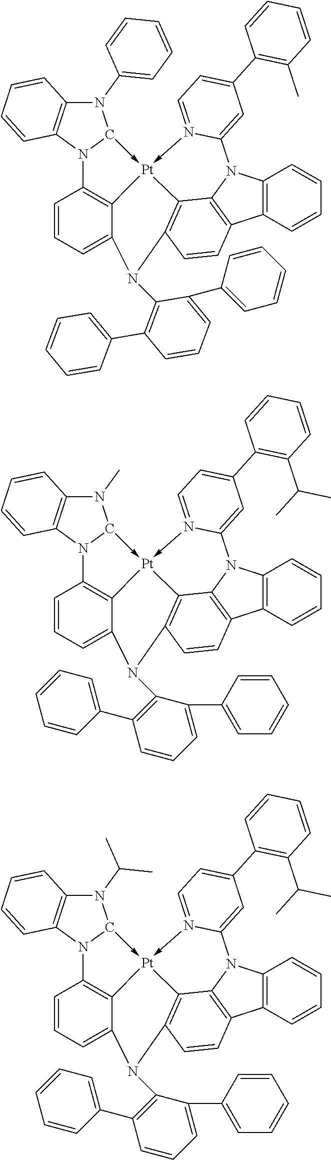

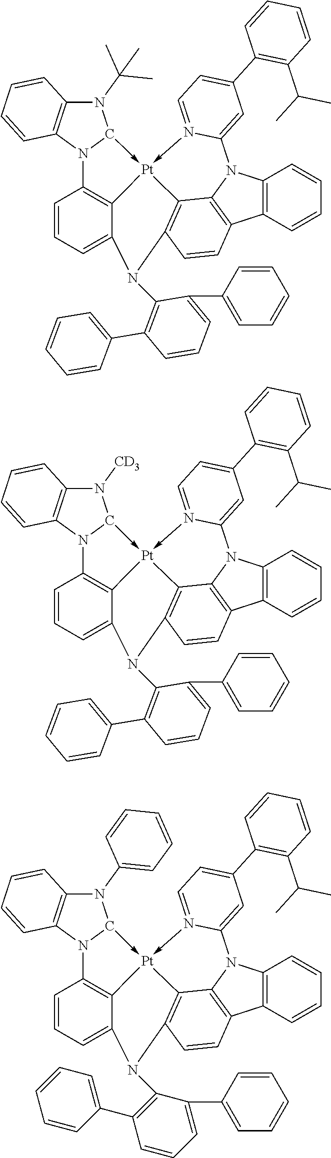

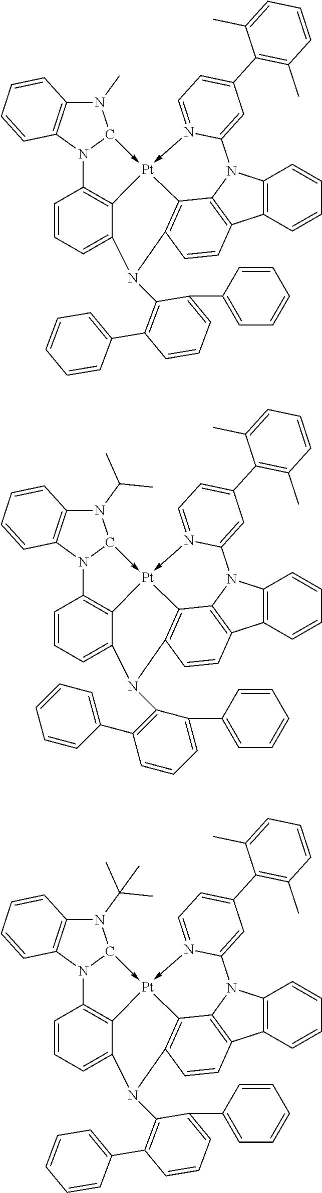

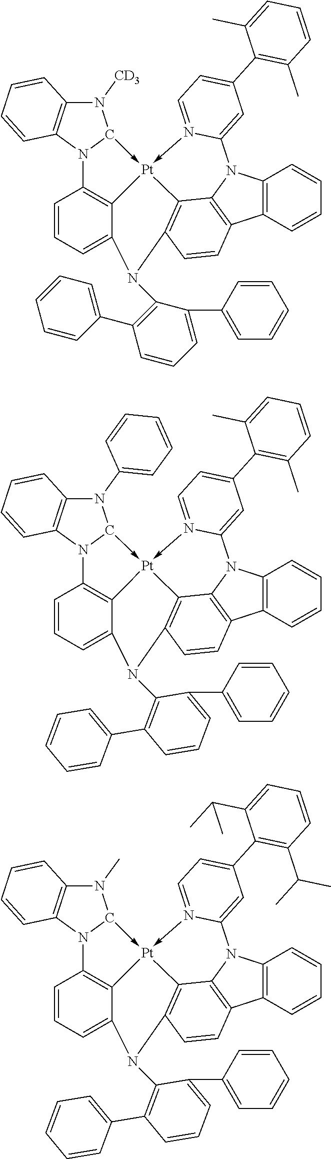

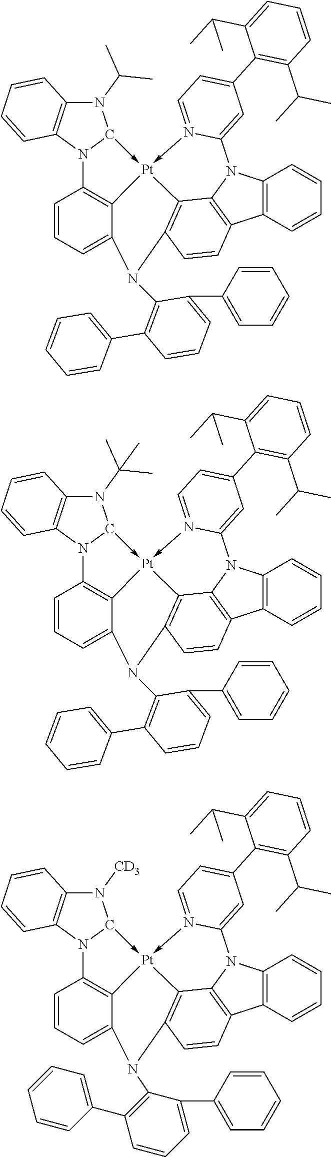

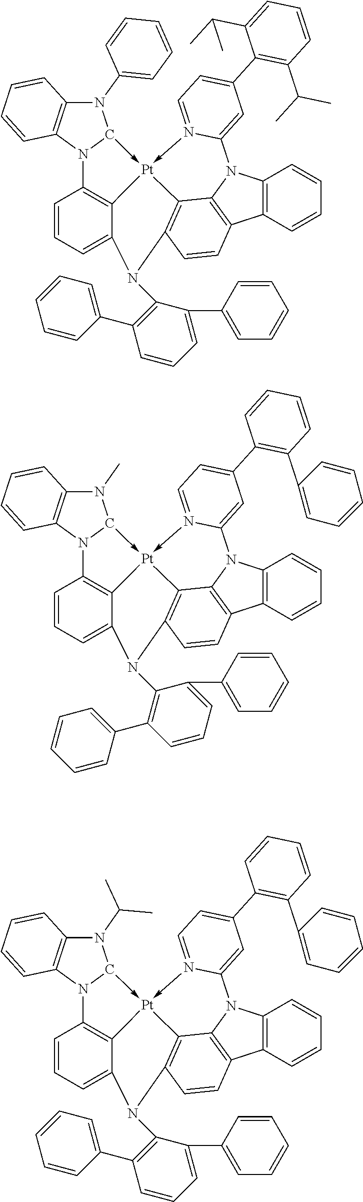

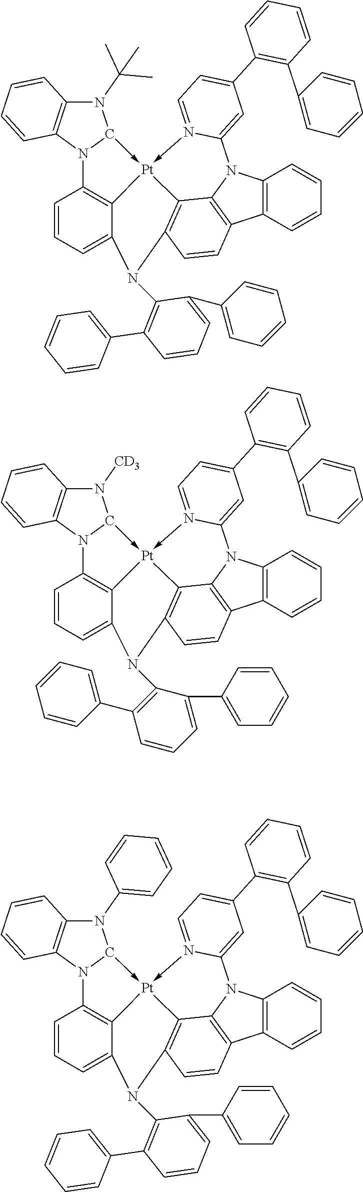

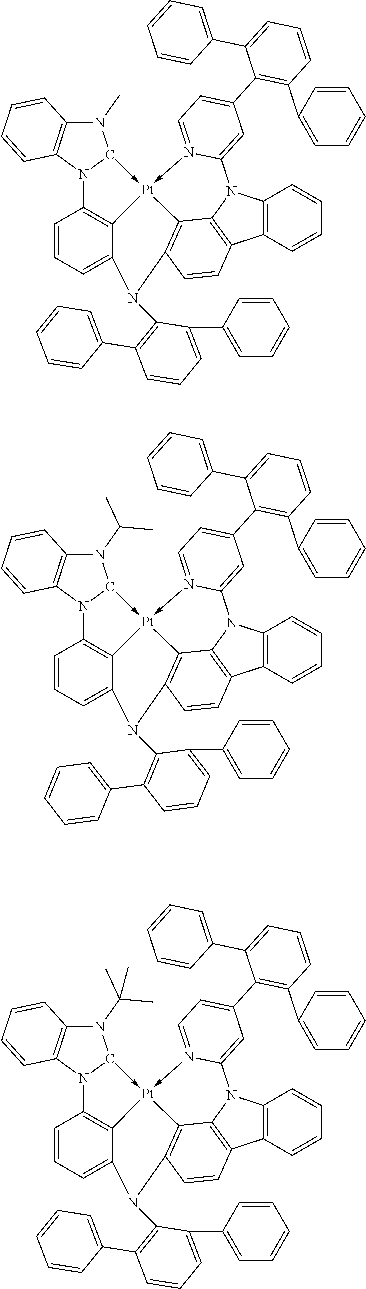

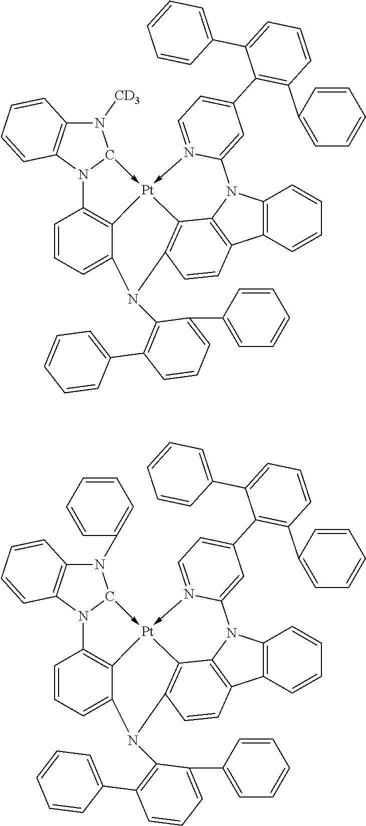

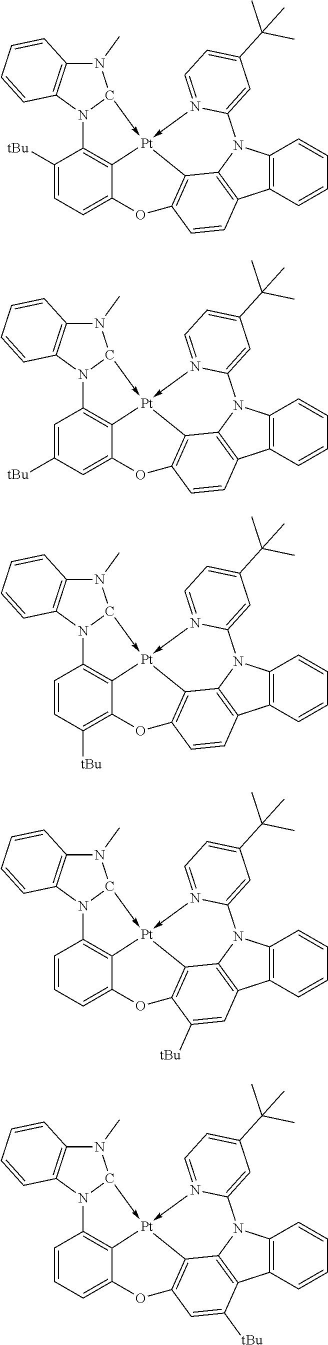

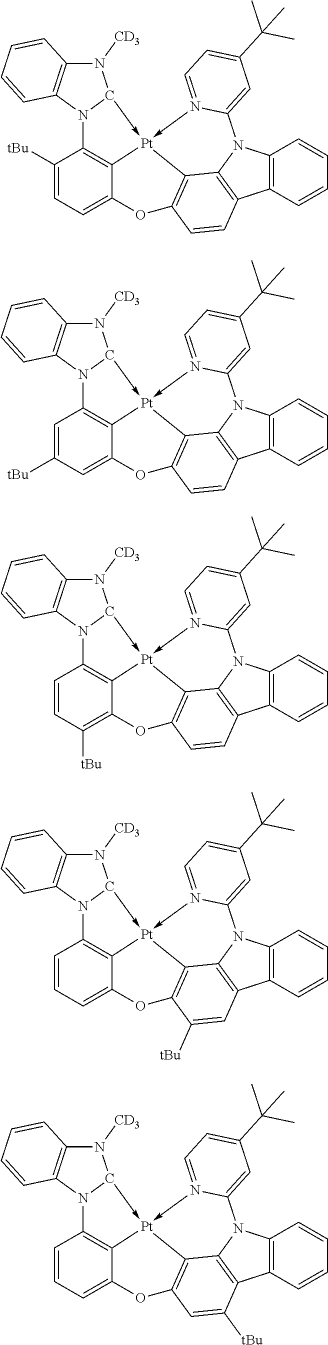

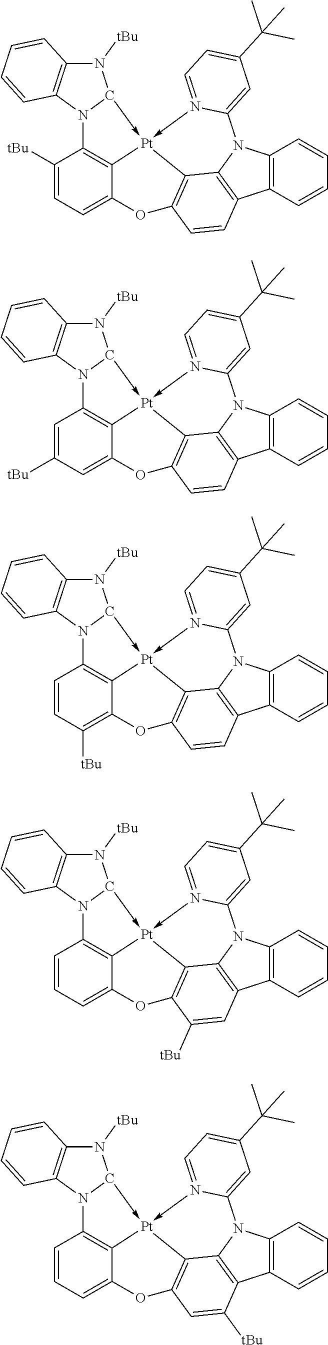

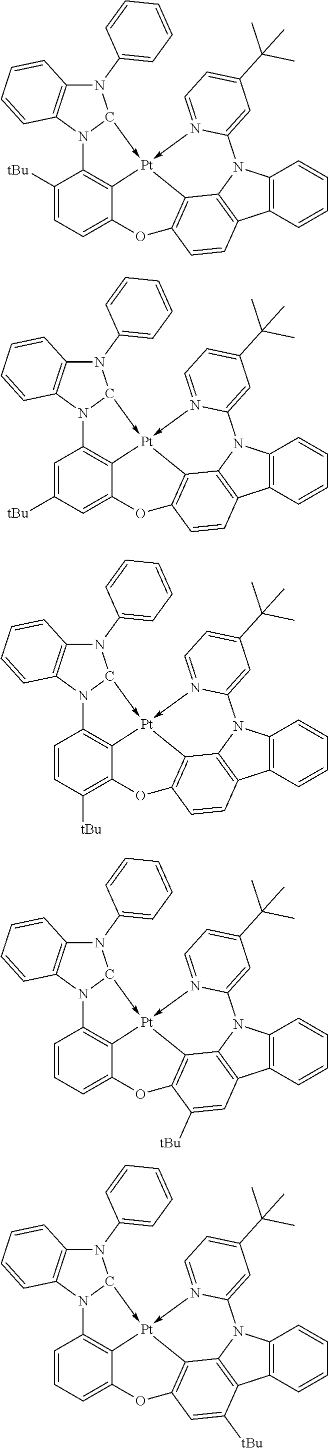

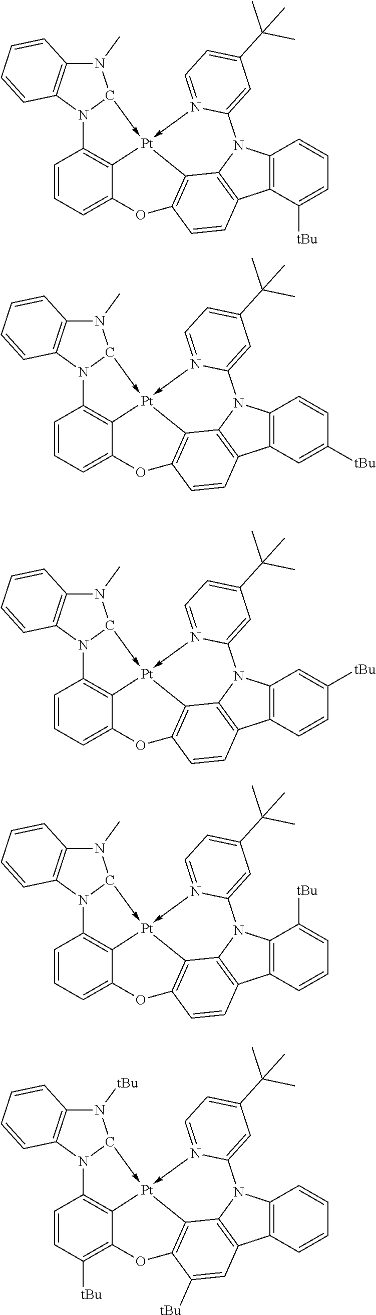

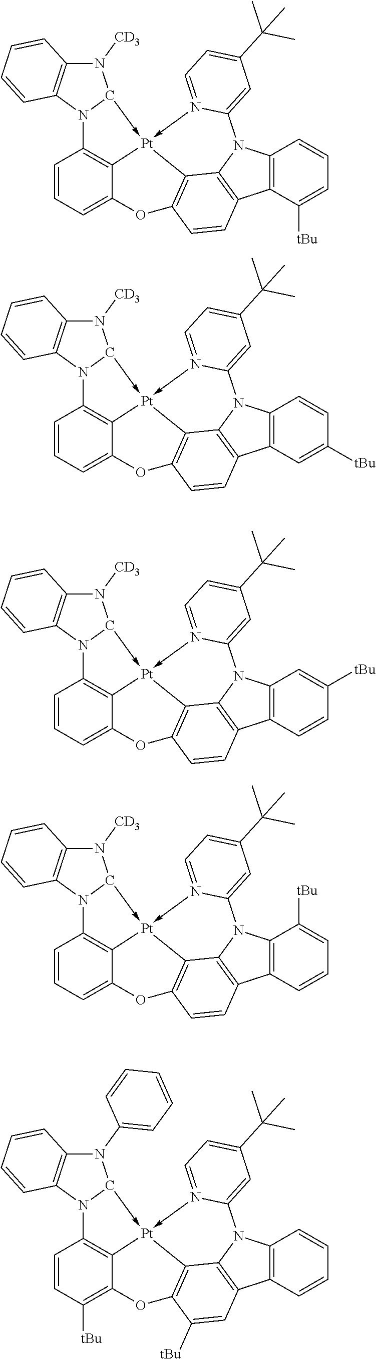

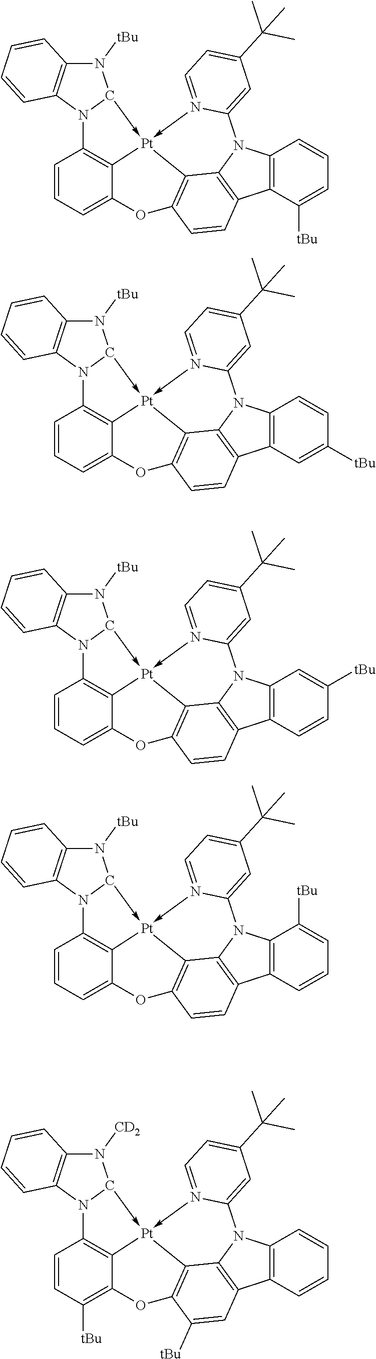

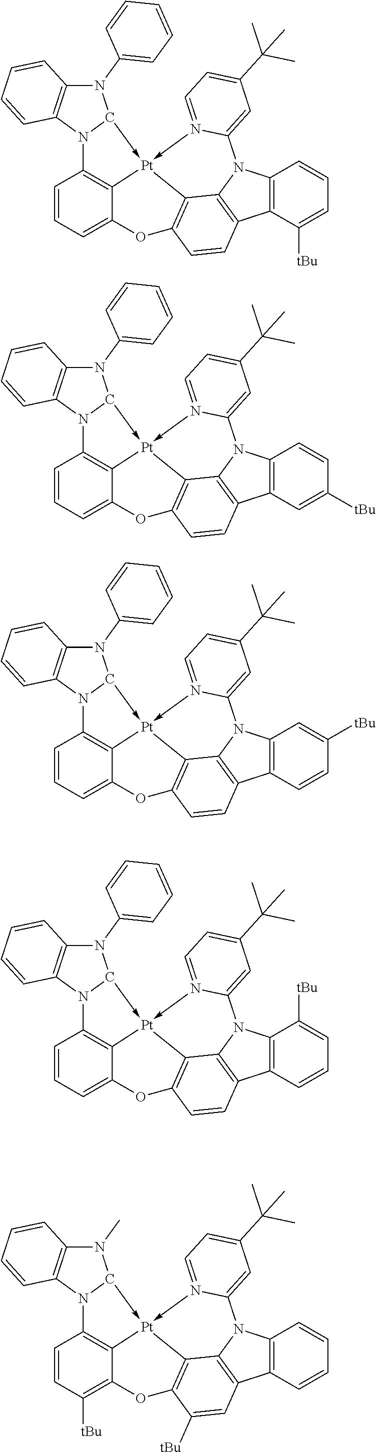

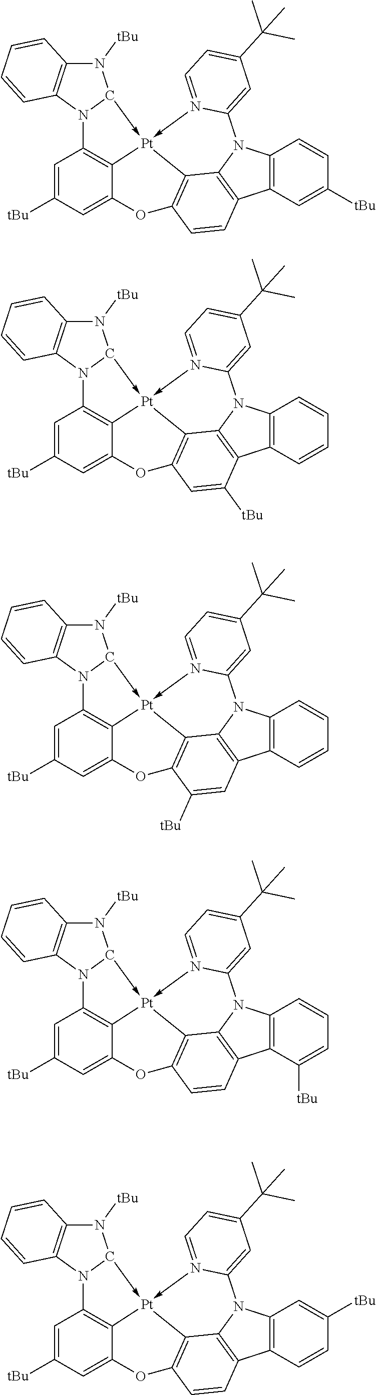

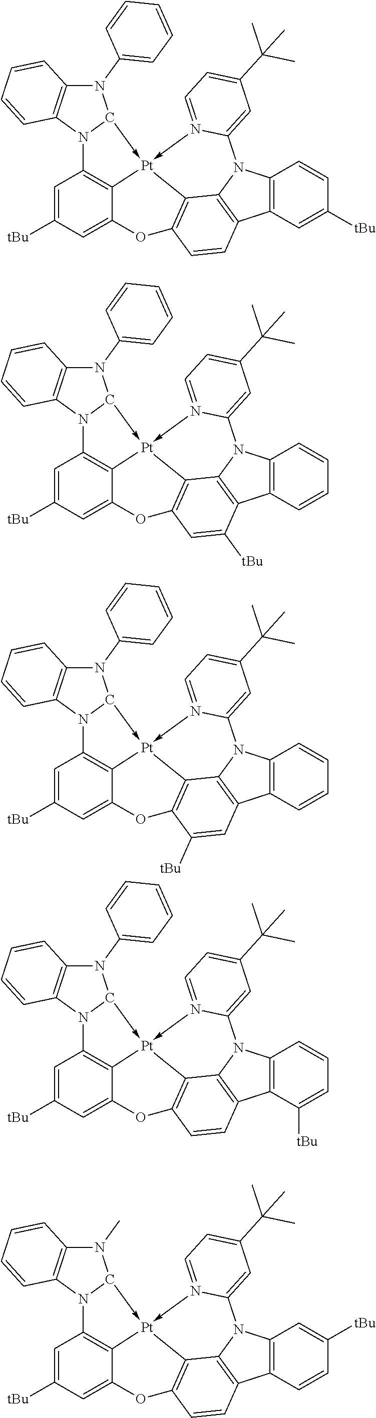

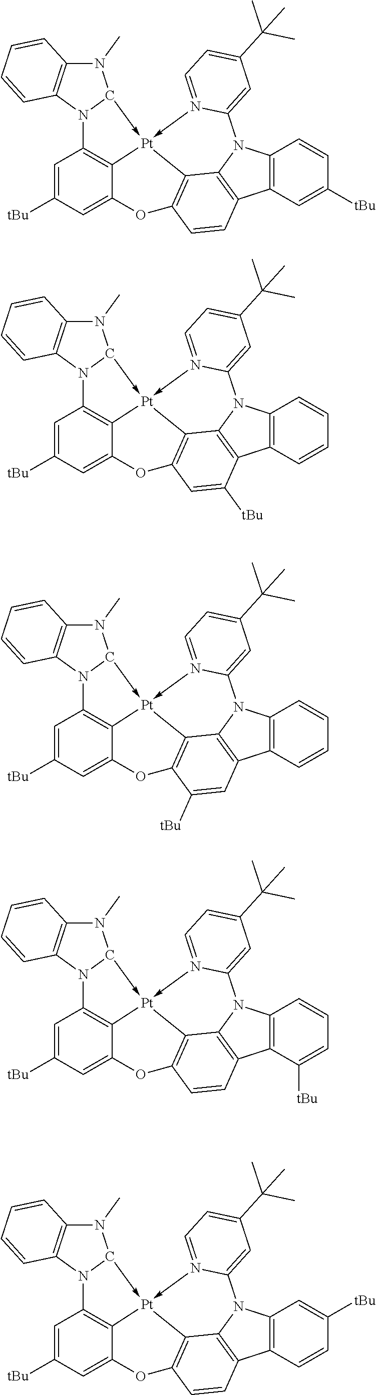

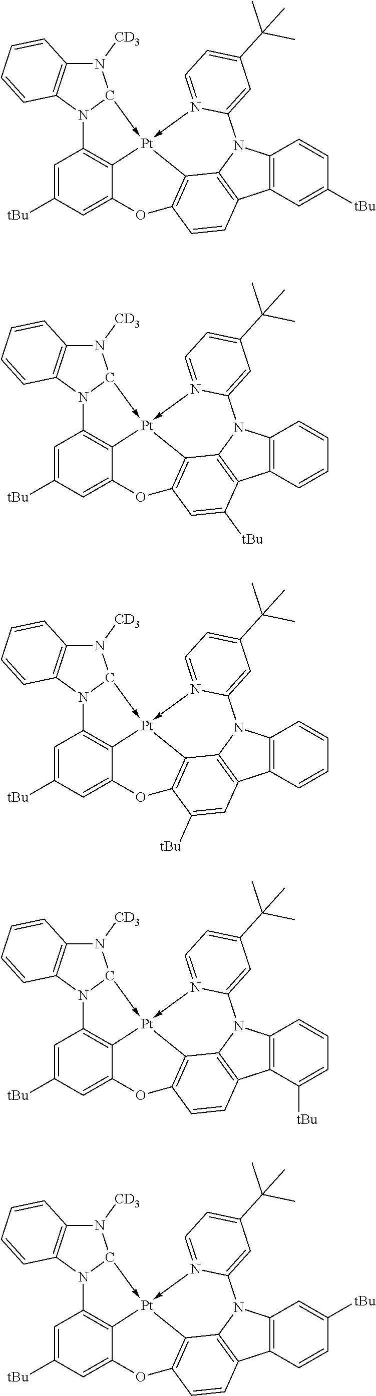

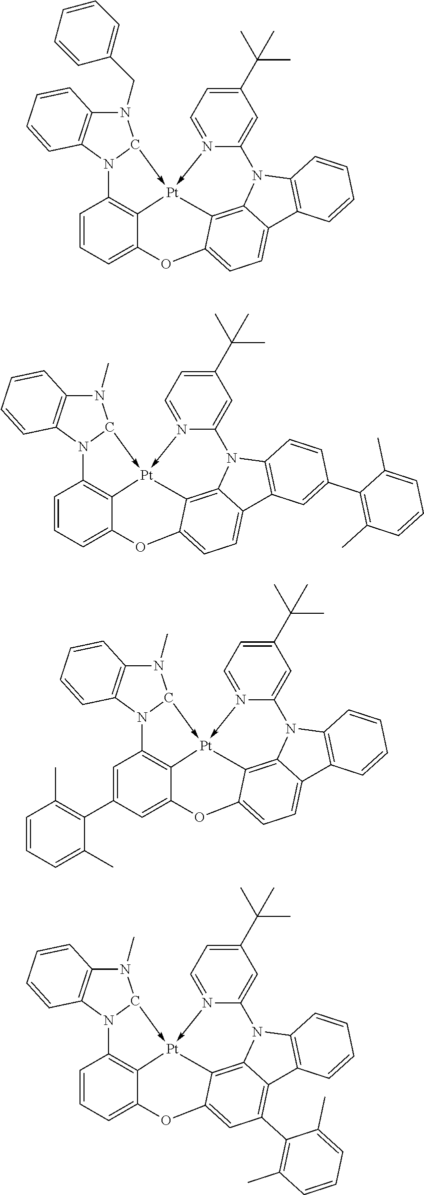

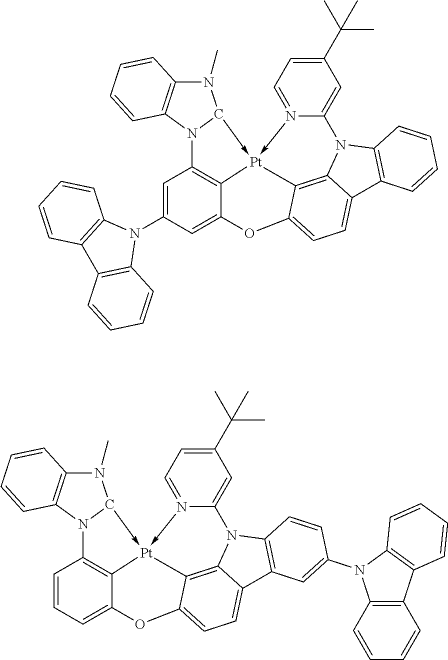

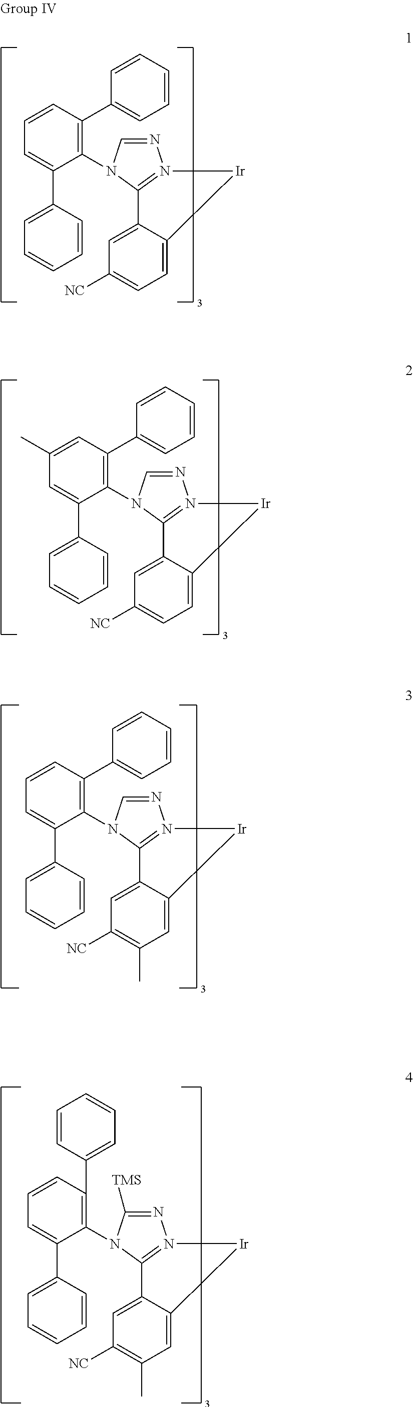

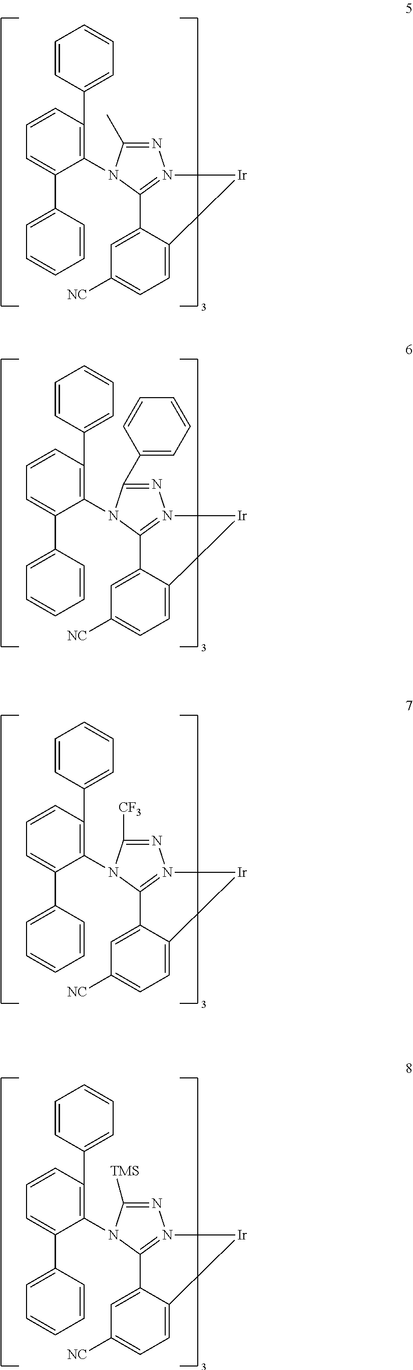

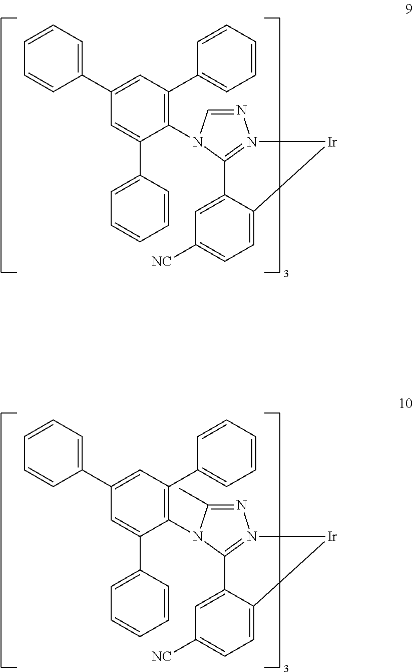

### Group V

A compound represented by Formula 1:

(L₁₀₁)ₙ₁₀₁—M₁₀₁—(L₁₀₂)ₘ₁₀₁ Formula A

wherein, in Formula A, L₁₀₁, n101, M₁₀₁, L₁₀₂, and m101 may be understood by referring to Tables 11 to 13:

TABLE 11

| Compound | L₁₀₁ | n101 | M₁₀₁ | L₁₀₂ | m101 |
|---|---|---|---|---|---|
| BD001 | LM1 | 3 | Ir | — | 0 |
| BD002 | LM2 | 3 | Ir | — | 0 |
| BD003 | LM3 | 3 | Ir | — | 0 |
| BD004 | LM4 | 3 | Ir | — | 0 |
| BD005 | LM5 | 3 | Ir | — | 0 |
| BD006 | LM6 | 3 | Ir | — | 0 |
| BD007 | LM7 | 3 | Ir | — | 0 |
| BD008 | LM8 | 3 | Ir | — | 0 |
| BD009 | LM9 | 3 | Ir | — | 0 |
| BD010 | LM10 | 3 | Ir | — | 0 |
| BD011 | LM11 | 3 | Ir | — | 0 |
| BD012 | LM12 | 3 | Ir | — | 0 |
| BD013 | LM13 | 3 | Ir | — | 0 |
| BD014 | LM14 | 3 | Ir | — | 0 |
| BD015 | LM15 | 3 | Ir | — | 0 |
| BD016 | LM16 | 3 | Ir | — | 0 |
| BD017 | LM17 | 3 | Ir | — | 0 |
| BD018 | LM18 | 3 | Ir | — | 0 |
| BD019 | LM19 | 3 | Ir | — | 0 |
| BD020 | LM20 | 3 | Ir | — | 0 |
| BD021 | LM21 | 3 | Ir | — | 0 |
| BD022 | LM22 | 3 | Ir | — | 0 |
| BD023 | LM23 | 3 | Ir | — | 0 |
| BD024 | LM24 | 3 | Ir | — | 0 |
| BD025 | LM25 | 3 | Ir | — | 0 |
| BD026 | LM26 | 3 | Ir | — | 0 |
| BD027 | LM27 | 3 | Ir | — | 0 |
| BD028 | LM28 | 3 | Ir | — | 0 |
| BD029 | LM29 | 3 | Ir | — | 0 |
| BD030 | LM30 | 3 | Ir | — | 0 |
| BD031 | LM31 | 3 | Ir | — | 0 |
| BD032 | LM32 | 3 | Ir | — | 0 |
| BD033 | LM33 | 3 | Ir | — | 0 |
| BD034 | LM34 | 3 | Ir | — | 0 |
| BD035 | LM35 | 3 | Ir | — | 0 |
| BD036 | LM36 | 3 | Ir | — | 0 |
| BD037 | LM37 | 3 | Ir | — | 0 |
| BD038 | LM38 | 3 | Ir | — | 0 |
| BD039 | LM39 | 3 | Ir | — | 0 |
| BD040 | LM40 | 3 | Ir | — | 0 |
| BD041 | LM41 | 3 | Ir | — | 0 |
| BD042 | LM42 | 3 | Ir | — | 0 |
| BD043 | LM43 | 3 | Ir | — | 0 |
| BD044 | LM44 | 3 | Ir | — | 0 |
| BD045 | LM45 | 3 | Ir | — | 0 |
| BD046 | LM46 | 3 | Ir | — | 0 |
| BD047 | LM47 | 3 | Ir | — | 0 |
| BD048 | LM48 | 3 | Ir | — | 0 |
| BD049 | LM49 | 3 | Ir | — | 0 |
| BD050 | LM50 | 3 | Ir | — | 0 |
| BD051 | LM51 | 3 | Ir | — | 0 |
| BD052 | LM52 | 3 | Ir | — | 0 |
| BD053 | LM53 | 3 | Ir | — | 0 |
| BD054 | LM54 | 3 | Ir | — | 0 |
| BD055 | LM55 | 3 | Ir | — | 0 |
| BD056 | LM56 | 3 | Ir | — | 0 |
| BD057 | LM57 | 3 | Ir | — | 0 |
| BD058 | LM58 | 3 | Ir | — | 0 |
| BD059 | LM59 | 3 | Ir | — | 0 |
| BD060 | LM60 | 3 | Ir | — | 0 |
| BD061 | LM61 | 3 | Ir | — | 0 |
| BD062 | LM62 | 3 | Ir | — | 0 |
| BD063 | LM63 | 3 | Ir | — | 0 |
| BD064 | LM64 | 3 | Ir | — | 0 |
| BD065 | LM65 | 3 | Ir | — | 0 |
| BD066 | LM66 | 3 | Ir | — | 0 |
| BD067 | LM67 | 3 | Ir | — | 0 |
| BD068 | LM68 | 3 | Ir | — | 0 |
| BD069 | LM69 | 3 | Ir | — | 0 |
| BD070 | LM70 | 3 | Ir | — | 0 |
| BD071 | LM71 | 3 | Ir | — | 0 |
| BD072 | LM72 | 3 | Ir | — | 0 |
| BD073 | LM73 | 3 | Ir | — | 0 |
| BD074 | LM74 | 3 | Ir | — | 0 |
| BD075 | LM75 | 3 | Ir | — | 0 |
| BD076 | LM76 | 3 | Ir | — | 0 |
| BD077 | LM77 | 3 | Ir | — | 0 |
| BD078 | LM78 | 3 | Ir | — | 0 |
| BD079 | LM79 | 3 | Ir | — | 0 |
| BD080 | LM80 | 3 | Ir | — | 0 |
| BD081 | LM81 | 3 | Ir | — | 0 |
| BD082 | LM82 | 3 | Ir | — | 0 |
| BD083 | LM83 | 3 | Ir | — | 0 |
| BD084 | LM84 | 3 | Ir | — | 0 |
| BD085 | LM85 | 3 | Ir | — | 0 |
| BD086 | LM86 | 3 | Ir | — | 0 |
| BD087 | LM87 | 3 | Ir | — | 0 |
| BD088 | LM88 | 3 | Ir | — | 0 |
| BD089 | LM89 | 3 | Ir | — | 0 |
| BD090 | LM90 | 3 | Ir | — | 0 |
| BD091 | LM91 | 3 | Ir | — | 0 |
| BD092 | LM92 | 3 | Ir | — | 0 |
| BD093 | LM93 | 3 | Ir | — | 0 |
| BD094 | LM94 | 3 | Ir | — | 0 |
| BD095 | LM95 | 3 | Ir | — | 0 |
| BD096 | LM96 | 3 | Ir | — | 0 |
| BD097 | LM97 | 3 | Ir | — | 0 |
| BD098 | LM98 | 3 | Ir | — | 0 |
| BD099 | LM99 | 3 | Ir | — | 0 |
| BD100 | LM100 | 3 | Ir | — | 0 |

TABLE 12

| Compound | L₁₀₁ | n101 | M₁₀₁ | L₁₀₂ | m101 |
|---|---|---|---|---|---|
| BD101 | LM101 | 3 | Ir | — | 0 |
| BD102 | LM102 | 3 | Ir | — | 0 |
| BD103 | LM103 | 3 | Ir | — | 0 |
| BD104 | LM104 | 3 | Ir | — | 0 |
| BD105 | LM105 | 3 | Ir | — | 0 |
| BD106 | LM106 | 3 | Ir | — | 0 |
| BD107 | LM107 | 3 | Ir | — | 0 |
| BD108 | LM108 | 3 | Ir | — | 0 |
| BD109 | LM109 | 3 | Ir | — | 0 |
| BD110 | LM110 | 3 | Ir | — | 0 |
| BD111 | LM111 | 3 | Ir | — | 0 |
| BD112 | LM112 | 3 | Ir | — | 0 |
| BD113 | LM113 | 3 | Ir | — | 0 |
| BD114 | LM114 | 3 | Ir | — | 0 |
| BD115 | LM115 | 3 | Ir | — | 0 |
| BD116 | LM116 | 3 | Ir | — | 0 |
| BD117 | LM117 | 3 | Ir | — | 0 |
| BD118 | LM118 | 3 | Ir | — | 0 |
| BD119 | LM119 | 3 | Ir | — | 0 |
| BD120 | LM120 | 3 | Ir | — | 0 |
| BD121 | LM121 | 3 | Ir | — | 0 |
| BD122 | LM122 | 3 | Ir | — | 0 |
| BD123 | LM123 | 3 | Ir | — | 0 |
| BD124 | LM124 | 3 | Ir | — | 0 |
| BD125 | LM125 | 3 | Ir | — | 0 |
| BD126 | LM126 | 3 | Ir | — | 0 |
| BD127 | LM127 | 3 | Ir | — | 0 |
| BD128 | LM128 | 3 | Ir | — | 0 |
| BD129 | LM129 | 3 | Ir | — | 0 |
| BD130 | LM130 | 3 | Ir | — | 0 |
| BD131 | LM131 | 3 | Ir | — | 0 |
| BD132 | LM132 | 3 | Ir | — | 0 |
| BD133 | LM133 | 3 | Ir | — | 0 |
| BD134 | LM134 | 3 | Ir | — | 0 |
| BD135 | LM135 | 3 | Ir | — | 0 |
| BD136 | LM136 | 3 | Ir | — | 0 |
| BD137 | LM137 | 3 | Ir | — | 0 |
| BD138 | LM138 | 3 | Ir | — | 0 |
| BD139 | LM139 | 3 | Ir | — | 0 |
| BD140 | LM140 | 3 | Ir | — | 0 |
| BD141 | LM141 | 3 | Ir | — | 0 |
| BD142 | LM142 | 3 | Ir | — | 0 |
| BD143 | LM143 | 3 | Ir | — | 0 |
| BD144 | LM144 | 3 | Ir | — | 0 |
| BD145 | LM145 | 3 | Ir | — | 0 |
| BD146 | LM146 | 3 | Ir | — | 0 |
| BD147 | LM147 | 3 | Ir | — | 0 |
| BD148 | LM148 | 3 | Ir | — | 0 |
| BD149 | LM149 | 3 | Ir | — | 0 |
| BD150 | LM150 | 3 | Ir | — | 0 |
| BD151 | LM151 | 3 | Ir | — | 0 |
| BD152 | LM152 | 3 | Ir | — | 0 |
| BD153 | LM153 | 3 | Ir | — | 0 |
| BD154 | LM154 | 3 | Ir | — | 0 |
| BD155 | LM155 | 3 | Ir | — | 0 |
| BD156 | LM156 | 3 | Ir | — | 0 |
| BD157 | LM157 | 3 | Ir | — | 0 |
| BD158 | LM158 | 3 | Ir | — | 0 |
| BD159 | LM159 | 3 | Ir | — | 0 |
| BD160 | LM160 | 3 | Ir | — | 0 |
| BD161 | LM161 | 3 | Ir | — | 0 |
| BD162 | LM162 | 3 | Ir | — | 0 |
| BD163 | LM163 | 3 | Ir | — | 0 |
| BD164 | LM164 | 3 | Ir | — | 0 |
| BD165 | LM165 | 3 | Ir | — | 0 |
| BD166 | LM166 | 3 | Ir | — | 0 |
| BD167 | LM167 | 3 | Ir | — | 0 |
| BD168 | LM168 | 3 | Ir | — | 0 |
| BD169 | LM169 | 3 | Ir | — | 0 |
| BD170 | LM170 | 3 | Ir | — | 0 |
| BD171 | LM171 | 3 | Ir | — | 0 |
| BD172 | LM172 | 3 | Ir | — | 0 |
| BD173 | LM173 | 3 | Ir | — | 0 |
| BD174 | LM174 | 3 | Ir | — | 0 |
| BD175 | LM175 | 3 | Ir | — | 0 |
| BD176 | LM176 | 3 | Ir | — | 0 |
| BD177 | LM177 | 3 | Ir | — | 0 |
| BD178 | LM178 | 3 | Ir | — | 0 |
| BD179 | LM179 | 3 | Ir | — | 0 |
| BD180 | LM180 | 3 | Ir | — | 0 |
| BD181 | LM181 | 3 | Ir | — | 0 |
| BD182 | LM182 | 3 | Ir | — | 0 |
| BD183 | LM183 | 3 | Ir | — | 0 |
| BD184 | LM184 | 3 | Ir | — | 0 |
| BD185 | LM185 | 3 | Ir | — | 0 |
| BD186 | LM186 | 3 | Ir | — | 0 |
| BD187 | LM187 | 3 | Ir | — | 0 |
| BD188 | LM188 | 3 | Ir | — | 0 |
| BD189 | LM189 | 3 | Ir | — | 0 |
| BD190 | LM190 | 3 | Ir | — | 0 |
| BD191 | LM191 | 3 | Ir | — | 0 |
| BD192 | LM192 | 3 | Ir | — | 0 |
| BD193 | LM193 | 3 | Ir | — | 0 |
| BD194 | LM194 | 3 | Ir | — | 0 |
| BD195 | LM195 | 3 | Ir | — | 0 |
| BD196 | LM196 | 3 | Ir | — | 0 |
| BD197 | LM197 | 3 | Ir | — | 0 |
| BD198 | LM198 | 3 | Ir | — | 0 |
| BD199 | LM199 | 3 | Ir | — | 0 |
| BD200 | LM200 | 3 | Ir | — | 0 |

TABLE 13

| Compound | L₁₀₁ | n101 | M₁₀₁ | L₁₀₂ | m101 |
|---|---|---|---|---|---|
| BD201 | LM201 | 3 | Ir | — | 0 |
| BD202 | LM202 | 3 | Ir | — | 0 |
| BD203 | LM203 | 3 | Ir | — | 0 |
| BD204 | LM204 | 3 | Ir | — | 0 |
| BD205 | LM205 | 3 | Ir | — | 0 |
| BD206 | LM206 | 3 | Ir | — | 0 |
| BD207 | LM207 | 3 | Ir | — | 0 |
| BD208 | LM208 | 3 | Ir | — | 0 |
| BD209 | LM209 | 3 | Ir | — | 0 |
| BD210 | LM210 | 3 | Ir | — | 0 |
| BD211 | LM211 | 3 | Ir | — | 0 |
| BD212 | LM212 | 3 | Ir | — | 0 |
| BD213 | LM213 | 3 | Ir | — | 0 |
| BD214 | LM214 | 3 | Ir | — | 0 |
| BD215 | LM215 | 3 | Ir | — | 0 |
| BD216 | LM216 | 3 | Ir | — | 0 |
| BD217 | LM217 | 3 | Ir | — | 0 |
| BD218 | LM218 | 3 | Ir | — | 0 |
| BD219 | LM219 | 3 | Ir | — | 0 |
| BD220 | LM220 | 3 | Ir | — | 0 |
| BD221 | LM221 | 3 | Ir | — | 0 |
| BD222 | LM222 | 3 | Ir | — | 0 |
| BD223 | LM223 | 3 | Ir | — | 0 |
| BD224 | LM224 | 3 | Ir | — | 0 |
| BD225 | LM225 | 3 | Ir | — | 0 |
| BD226 | LM226 | 3 | Ir | — | 0 |
| BD227 | LM227 | 3 | Ir | — | 0 |
| BD228 | LM228 | 3 | Ir | — | 0 |
| BD229 | LM229 | 3 | Ir | — | 0 |
| BD230 | LM230 | 3 | Ir | — | 0 |
| BD231 | LM231 | 3 | Ir | — | 0 |
| BD232 | LM232 | 3 | Ir | — | 0 |
| BD233 | LM233 | 3 | Ir | — | 0 |
| BD234 | LM234 | 3 | Ir | — | 0 |
| BD235 | LM235 | 3 | Ir | — | 0 |
| BD236 | LM236 | 3 | Ir | — | 0 |
| BD237 | LM237 | 3 | Ir | — | 0 |
| BD238 | LM238 | 3 | Ir | — | 0 |
| BD239 | LM239 | 3 | Ir | — | 0 |
| BD240 | LM240 | 3 | Ir | — | 0 |
| BD241 | LM241 | 3 | Ir | — | 0 |
| BD242 | LM242 | 3 | Ir | — | 0 |
| BD243 | LM243 | 3 | Ir | — | 0 |
| BD244 | LFM1 | 3 | Ir | — | 0 |
| BD245 | LFM2 | 3 | Ir | — | 0 |
| BD246 | LFM3 | 3 | Ir | — | 0 |
| BD247 | LFM4 | 3 | Ir | — | 0 |
| BD248 | LFM5 | 3 | Ir | — | 0 |
| BD249 | LFM6 | 3 | Ir | — | 0 |
| BD250 | LFM7 | 3 | Ir | — | 0 |
| BD251 | LFP1 | 3 | Ir | — | 0 |
| BD252 | LFP2 | 3 | Ir | — | 0 |
| BD253 | LFP3 | 3 | Ir | — | 0 |
| BD254 | LFP4 | 3 | Ir | — | 0 |
| BD255 | LFP5 | 3 | Ir | — | 0 |
| BD256 | LFP6 | 3 | Ir | — | 0 |
| BD257 | LFP7 | 3 | Ir | — | 0 |
| BD258 | LM47 | 2 | Ir | AN1 | 1 |
| BD259 | LM47 | 2 | Ir | AN2 | 1 |
| BD260 | LM47 | 2 | Ir | AN3 | 1 |
| BD261 | LM47 | 2 | Ir | AN4 | 1 |
| BD262 | LM47 | 2 | Ir | AN5 | 1 |
| BD263 | LM11 | 2 | Pt | — | 0 |
| BD264 | LM13 | 2 | Pt | — | 0 |
| BD265 | LM15 | 2 | Pt | — | 0 |
| BD266 | LM45 | 2 | Pt | — | 0 |
| BD267 | LM47 | 2 | Pt | — | 0 |
| BD268 | LM49 | 2 | Pt | — | 0 |
| BD269 | LM98 | 2 | Pt | — | 0 |
| BD270 | LM100 | 2 | Pt | — | 0 |
| BD271 | LM102 | 2 | Pt | — | 0 |
| BD272 | LM132 | 2 | Pt | — | 0 |
| BD273 | LM134 | 2 | Pt | — | 0 |
| BD274 | LM136 | 2 | Pt | — | 0 |
| BD275 | LM151 | 2 | Pt | — | 0 |
| BD276 | LM153 | 2 | Pt | — | 0 |
| BD277 | LM158 | 2 | Pt | — | 0 |
| BD278 | LM180 | 2 | Pt | — | 0 |
| BD279 | LM182 | 2 | Pt | — | 0 |
| BD280 | LM187 | 2 | Pt | — | 0 |
| BD281 | LM201 | 2 | Pt | — | 0 |
| BD282 | LM206 | 2 | Pt | — | 0 |
| BD283 | LM211 | 2 | Pt | — | 0 |
| BD284 | LM233 | 2 | Pt | — | 0 |
| BD285 | LM235 | 2 | Pt | — | 0 |
| BD286 | LM240 | 2 | Pt | — | 0 |
| BD287 | LFM5 | 2 | Pt | — | 0 |
| BD288 | LFM6 | 2 | Pt | — | 0 |
| BD289 | LFM7 | 2 | Pt | — | 0 |
| BD290 | LFP5 | 2 | Pt | — | 0 |
| BD291 | LFP6 | 2 | Pt | — | 0 |
| BD292 | LFP7 | 2 | Pt | — | 0 |
| BD293 | LM47 | 1 | Pt | AN1 | 1 |
| BD294 | LM47 | 1 | Pt | AN2 | 1 |
| BD295 | LM47 | 1 | Pt | AN3 | 1 |
| BD296 | LM47 | 1 | Pt | AN4 | 1 |
| BD297 | LM47 | 1 | Pt | AN5 | 1 |

In Tables 11 to 13, LM1 to LM243 may be understood by referring to Formulae 1-i to 1-3 and Tables 14 to 16:

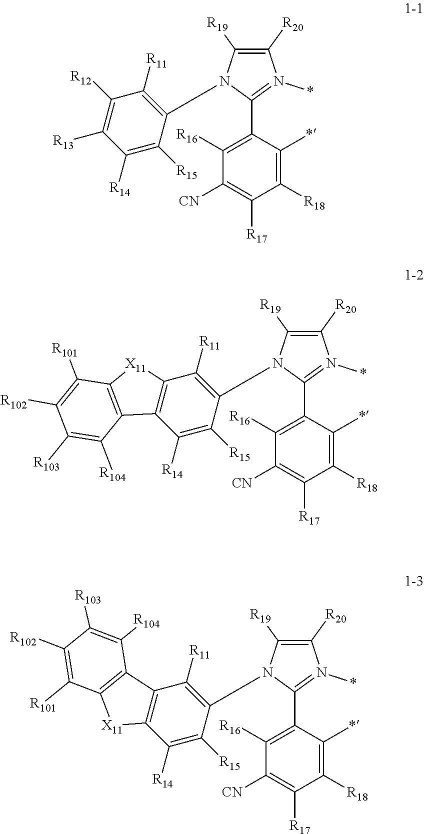

TABLE 14
Formula 1-1

| Ligand | R₁₁ | R₁₂ | R₁₃ | R₁₄ | R₁₅ | R₁₆ | R₁₇ | R₁₈ | R₁₉ | R₂₀ |
|---|---|---|---|---|---|---|---|---|---|---|
| LM1 | X1 | H | X3 | H | X1 | H | H | H | H | D |
| LM2 | X1 | H | X3 | H | X1 | H | H | H | D | H |
| LM3 | X1 | H | X3 | H | X1 | H | H | H | D | D |
| LM4 | Y1 | H | X3 | H | Y1 | H | H | H | D | D |
| LM5 | Y2 | H | X3 | H | Y2 | H | H | H | D | D |
| LM6 | Y3 | H | X3 | H | Y3 | H | H | H | D | D |
| LM7 | Y3 | D | X3 | D | Y3 | H | H | H | D | D |
| LM8 | Y3 | D | X3 | D | Y3 | D | H | H | D | D |
| LM9 | Y3 | D | X3 | D | Y3 | D | D | H | D | D |
| LM10 | Y3 | D | X3 | D | Y3 | D | D | D | D | D |
| LM11 | Y3 | D | Y11 | D | Y3 | D | D | D | D | D |
| LM12 | Y3 | D | Y11 | D | Y3 | H | X1 | H | D | D |
| LM13 | Y3 | D | Y11 | D | Y3 | D | Y3 | D | D | D |
| LM14 | Y3 | D | Y11 | D | Y3 | H | X4 | H | D | D |
| LM15 | Y3 | D | Y11 | D | Y3 | D | Y12 | D | D | D |
| LM16 | X2 | H | X3 | H | X2 | H | H | H | H | D |
| LM17 | X2 | H | X3 | H | X2 | H | H | H | D | H |
| LM18 | X2 | H | X3 | H | X2 | H | H | H | D | D |
| LM19 | Y4 | H | X3 | H | Y4 | H | H | H | D | D |
| LM20 | Y5 | H | X3 | H | Y5 | H | H | H | D | D |
| LM21 | Y6 | H | X3 | H | Y6 | H | H | H | D | D |
| LM22 | Y7 | H | X3 | H | Y7 | H | H | H | D | D |
| LM23 | Y8 | H | X3 | H | Y8 | H | H | H | D | D |
| LM24 | Y9 | H | X3 | H | Y9 | H | H | H | D | D |
| LM25 | Y10 | H | X3 | H | Y10 | H | H | H | D | D |
| LM26 | Y10 | D | X3 | D | Y10 | H | H | H | D | D |
| LM27 | Y10 | D | X3 | D | Y10 | D | H | H | D | D |
| LM28 | Y10 | D | X3 | D | Y10 | D | D | H | D | D |
| LM29 | Y10 | D | X3 | D | Y10 | D | D | D | D | D |
| LM30 | Y10 | D | Y11 | D | Y10 | D | D | D | D | D |
| LM31 | Y10 | D | Y11 | D | Y10 | H | X1 | H | D | D |
| LM32 | Y10 | D | Y11 | D | Y10 | D | Y3 | D | D | D |
| LM33 | Y10 | D | Y11 | D | Y10 | H | X4 | H | D | D |
| LM34 | Y10 | D | Y11 | D | Y10 | D | Y12 | D | D | D |
| LM35 | X1 | H | X4 | H | X1 | H | H | H | H | D |
| LM36 | X1 | H | X4 | H | X1 | H | H | H | D | H |
| LM37 | X1 | H | X4 | H | X1 | H | H | H | D | D |
| LM38 | Y1 | H | X4 | H | Y1 | H | H | H | D | D |
| LM39 | Y2 | H | X4 | H | Y2 | H | H | H | D | D |
| LM40 | Y3 | H | X4 | H | Y3 | H | H | H | D | D |
| LM41 | Y3 | D | X4 | D | Y3 | H | H | H | D | D |
| LM42 | Y3 | D | X4 | D | Y3 | D | H | H | D | D |
| LM43 | Y3 | D | X4 | D | Y3 | D | D | H | D | D |
| LM44 | Y3 | D | X4 | D | Y3 | D | D | D | D | D |
| LM45 | Y3 | D | Y12 | D | Y3 | D | D | D | D | D |
| LM46 | Y3 | D | Y12 | D | Y3 | H | X1 | H | D | D |
| LM47 | Y3 | D | Y12 | D | Y3 | D | Y3 | D | D | D |
| LM48 | Y3 | D | Y12 | D | Y3 | H | X4 | H | D | D |
| LM49 | Y3 | D | Y12 | D | Y3 | D | Y12 | D | D | D |
| LM50 | X2 | H | X4 | H | X2 | H | H | H | H | D |
| LM51 | X2 | H | X4 | H | X2 | H | H | H | D | H |
| LM52 | X2 | H | X4 | H | X2 | H | H | H | D | D |
| LM53 | Y4 | H | X4 | H | Y4 | H | H | H | D | D |
| LM54 | Y5 | H | X4 | H | Y5 | H | H | H | D | D |
| LM55 | Y6 | H | X4 | H | Y6 | H | H | H | D | D |
| LM56 | Y7 | H | X4 | H | Y7 | H | H | H | D | D |
| LM57 | Y8 | H | X4 | H | Y8 | H | H | H | D | D |
| LM58 | Y9 | H | X4 | H | Y9 | H | H | H | D | D |
| LM59 | Y10 | H | X4 | H | Y10 | H | H | H | D | D |
| LM60 | Y10 | D | X4 | D | Y10 | H | H | H | D | D |
| LM61 | Y10 | D | X4 | D | Y10 | D | H | H | D | D |
| LM62 | Y10 | D | X4 | D | Y10 | D | D | H | D | D |
| LM63 | Y10 | D | X4 | D | Y10 | D | D | D | D | D |
| LM64 | Y10 | D | Y12 | D | Y10 | D | D | D | D | D |
| LM65 | Y10 | D | Y12 | D | Y10 | H | X1 | H | D | D |
| LM66 | Y10 | D | Y12 | D | Y10 | D | Y3 | D | D | D |
| LM67 | Y10 | D | Y12 | D | Y10 | H | X4 | H | D | D |
| LM68 | Y10 | D | Y12 | D | Y10 | D | Y12 | D | D | D |
| LM69 | X1 | H | X5 | H | X1 | H | H | H | H | D |
| LM70 | X1 | H | X5 | H | X1 | H | H | H | D | H |
| LM71 | X1 | H | X5 | H | X1 | H | H | H | D | D |
| LM72 | Y1 | H | X5 | H | Y1 | H | H | H | D | D |
| LM73 | Y2 | H | X5 | H | Y2 | H | H | H | D | D |
| LM74 | Y3 | H | X5 | H | Y3 | H | H | H | D | D |
| LM75 | Y3 | D | X5 | D | Y3 | H | H | H | D | D |
| LM76 | Y3 | D | X5 | D | Y3 | D | H | H | D | D |
| LM77 | Y3 | D | X5 | D | Y3 | D | D | H | D | D |
| LM78 | Y3 | D | X5 | D | Y3 | D | D | D | D | D |
| LM79 | Y3 | D | Y13 | D | Y3 | D | D | D | D | D |
| LM80 | Y3 | D | Y13 | D | Y3 | H | X1 | H | D | D |
| LM81 | Y3 | D | Y13 | D | Y3 | D | Y3 | D | D | D |
| LM82 | Y3 | D | Y13 | D | Y3 | H | X4 | H | D | D |
| LM83 | Y3 | D | Y13 | D | Y3 | D | Y12 | D | D | D |
| LM84 | X2 | H | X5 | H | X2 | H | H | H | H | D |
| LM85 | X2 | H | X5 | H | X2 | H | H | H | D | H |
| LM86 | X2 | H | X5 | H | X2 | H | H | H | D | D |
| LM87 | Y4 | H | X5 | H | Y4 | H | H | H | D | D |
| LM88 | Y5 | H | X5 | H | Y5 | H | H | H | D | D |
| LM89 | Y6 | H | X5 | H | Y6 | H | H | H | D | D |
| LM90 | Y7 | H | X5 | H | Y7 | H | H | H | D | D |
| LM91 | Y8 | H | X5 | H | Y8 | H | H | H | D | D |
| LM92 | Y9 | H | X5 | H | Y9 | H | H | H | D | D |
| LM93 | Y10 | H | X5 | H | Y10 | H | H | H | D | D |
| LM94 | Y10 | D | X5 | D | Y10 | H | H | H | D | D |
| LM95 | Y10 | D | X5 | D | Y10 | D | H | H | D | D |
| LM96 | Y10 | D | X5 | D | Y10 | D | D | H | D | D |
| LM97 | Y10 | D | X5 | D | Y10 | D | D | D | D | D |
| LM98 | Y10 | D | Y13 | D | Y10 | D | D | D | D | D |
| LM99 | Y10 | D | Y13 | D | Y10 | H | X1 | H | D | D |
| LM100 | Y10 | D | Y13 | D | Y10 | D | Y3 | D | D | D |
| LM101 | Y10 | D | Y13 | D | Y10 | H | X4 | H | D | D |
| LM102 | Y10 | D | Y13 | D | Y10 | D | Y12 | D | D | D |
| LM103 | X1 | H | X6 | H | X1 | H | H | H | H | D |
| LM104 | X1 | H | X6 | H | X1 | H | H | H | D | H |
| LM105 | X1 | H | X6 | H | X1 | H | H | H | D | D |
| LM106 | Y1 | H | X6 | H | Y1 | H | H | H | D | D |
| LM107 | Y2 | H | X6 | H | Y2 | H | H | H | D | D |
| LM108 | Y3 | H | X6 | H | Y3 | H | H | H | D | D |
| LM109 | Y3 | D | X6 | D | Y3 | H | H | H | D | D |
| LM110 | Y3 | D | X6 | D | Y3 | D | H | H | D | D |
| LM111 | Y3 | D | X6 | D | Y3 | D | D | H | D | D |
| LM112 | Y3 | D | X6 | D | Y3 | D | D | D | D | D |
| LM113 | Y3 | D | Y14 | D | Y3 | D | D | D | D | D |
| LM114 | Y3 | D | Y14 | D | Y3 | H | X1 | H | D | D |
| LM115 | Y3 | D | Y14 | D | Y3 | D | Y3 | D | D | D |
| LM116 | Y3 | D | Y14 | D | Y3 | H | X4 | H | D | D |
| LM117 | Y3 | D | Y14 | D | Y3 | D | Y12 | D | D | D |
| LM118 | X2 | H | X6 | H | X2 | H | H | H | H | D |
| LM119 | X2 | H | X6 | H | X2 | H | H | H | D | H |
| LM120 | X2 | H | X6 | H | X2 | H | H | H | D | D |
| LM121 | Y4 | H | X6 | H | Y4 | H | H | H | D | D |
| LM122 | Y5 | H | X6 | H | Y5 | H | H | H | D | D |
| LM123 | Y6 | H | X6 | H | Y6 | H | H | H | D | D |
| LM124 | Y7 | H | X6 | H | Y7 | H | H | H | D | D |
| LM125 | Y8 | H | X6 | H | Y8 | H | H | H | D | D |
| LM126 | Y9 | H | X6 | H | Y9 | H | H | H | D | D |
| LM127 | Y10 | H | X6 | H | Y10 | H | H | H | D | D |
| LM128 | Y10 | D | X6 | D | Y10 | H | H | H | D | D |
| LM129 | Y10 | D | X6 | D | Y10 | D | H | H | D | D |
| LM130 | Y10 | D | X6 | D | Y10 | D | D | H | D | D |
| LM131 | Y10 | D | X6 | D | Y10 | D | D | D | D | D |
| LM132 | Y10 | D | Y14 | D | Y10 | D | D | D | D | D |
| LM133 | Y10 | D | Y14 | D | Y10 | H | X1 | H | D | D |
| LM134 | Y10 | D | Y14 | D | Y10 | D | Y3 | D | D | D |
| LM135 | Y10 | D | Y14 | D | Y10 | H | X4 | H | D | D |
| LM136 | Y10 | D | Y14 | D | Y10 | D | Y12 | D | D | D |
| LM137 | X1 | H | X7 | H | X1 | H | H | H | H | D |
| LM138 | X1 | H | X7 | H | X1 | H | H | H | D | H |
| LM139 | X1 | H | X7 | H | X1 | H | H | H | D | D |
| LM140 | Y1 | H | X7 | H | Y1 | H | H | H | D | D |
| LM141 | Y2 | H | X7 | H | Y2 | H | H | H | D | D |
| LM142 | Y3 | H | X7 | H | Y3 | H | H | H | D | D |
| LM143 | Y3 | D | X7 | D | Y3 | H | H | H | D | D |
| LM144 | Y3 | D | X7 | D | Y3 | D | H | H | D | D |
| LM145 | Y3 | D | X7 | D | Y3 | D | D | H | D | D |
| LM146 | Y3 | D | X7 | D | Y3 | D | D | D | D | D |
| LM147 | Y3 | D | X8 | D | Y3 | D | D | D | D | D |
| LM148 | Y3 | D | Y16 | D | Y3 | D | D | D | D | D |
| LM149 | Y3 | D | Y17 | D | Y3 | D | D | D | D | D |
| LM150 | Y3 | D | Y18 | D | Y3 | D | D | D | D | D |
| LM151 | Y3 | D | Y15 | D | Y3 | D | D | D | D | D |
| LM152 | Y3 | D | Y15 | D | Y3 | H | X1 | H | D | D |
| LM153 | Y3 | D | Y15 | D | Y3 | D | Y3 | D | D | D |
| LM154 | Y3 | D | Y16 | D | Y3 | D | Y3 | D | D | D |
| LM155 | Y3 | D | Y17 | D | Y3 | D | Y3 | D | D | D |
| LM156 | Y3 | D | Y18 | D | Y3 | D | Y3 | D | D | D |
| LM157 | Y3 | D | Y15 | D | Y3 | H | X4 | H | D | D |
| LM158 | Y3 | D | Y15 | D | Y3 | D | Y12 | D | D | D |
| LM159 | Y3 | D | Y16 | D | Y3 | D | Y12 | D | D | D |
| LM160 | Y3 | D | Y17 | D | Y3 | D | Y12 | D | D | D |
| LM161 | Y3 | D | Y18 | D | Y3 | D | Y12 | D | D | D |
| LM162 | X2 | H | X7 | H | X2 | H | H | H | H | D |
| LM163 | X2 | H | X7 | H | X2 | H | H | H | D | H |
| LM164 | X2 | H | X7 | H | X2 | H | H | H | D | D |
| LM165 | Y4 | H | X7 | H | Y4 | H | H | H | D | D |
| LM166 | Y5 | H | X7 | H | Y5 | H | H | H | D | D |
| LM167 | Y6 | H | X7 | H | Y6 | H | H | H | D | D |
| LM168 | Y7 | H | X7 | H | Y7 | H | H | H | D | D |
| LM169 | Y8 | H | X7 | H | Y8 | H | H | H | D | D |
| LM170 | Y9 | H | X7 | H | Y9 | H | H | H | D | D |
| LM171 | Y10 | H | X7 | H | Y10 | H | H | H | D | D |
| LM172 | Y10 | D | X7 | D | Y10 | H | H | H | D | D |
| LM173 | Y10 | D | X7 | D | Y10 | D | H | H | D | D |
| LM174 | Y10 | D | X7 | D | Y10 | D | D | H | D | D |
| LM175 | Y10 | D | X7 | D | Y10 | D | D | D | D | D |
| LM176 | Y10 | D | X8 | D | Y10 | D | D | D | D | D |
| LM177 | Y10 | D | Y16 | D | Y10 | D | D | D | D | D |
| LM178 | Y10 | D | Y17 | D | Y10 | D | D | D | D | D |
| LM179 | Y10 | D | Y18 | D | Y10 | D | D | D | D | D |
| LM180 | Y10 | D | Y15 | D | Y10 | D | D | D | D | D |
| LM181 | Y10 | D | Y15 | D | Y10 | H | X1 | H | D | D |
| LM182 | Y10 | D | Y15 | D | Y10 | D | Y3 | D | D | D |
| LM183 | Y10 | D | Y16 | D | Y10 | D | Y3 | D | D | D |
| LM184 | Y10 | D | Y17 | D | Y10 | D | Y3 | D | D | D |
| LM185 | Y10 | D | Y18 | D | Y10 | D | Y3 | D | D | D |
| LM186 | Y10 | D | Y15 | D | Y10 | H | X4 | H | D | D |
| LM187 | Y10 | D | Y15 | D | Y10 | D | Y12 | D | D | D |
| LM188 | Y10 | D | Y16 | D | Y10 | D | Y12 | D | D | D |
| LM189 | Y10 | D | Y17 | D | Y10 | D | Y12 | D | D | D |
| LM190 | Y10 | D | Y18 | D | Y10 | D | Y12 | D | D | D |
| LM191 | X1 | X7 | H | H | X1 | H | H | H | H | D |
| LM192 | X1 | X7 | H | H | X1 | H | H | H | D | H |
| LM193 | X1 | X7 | H | H | X1 | H | H | H | D | D |
| LM194 | Y1 | X7 | H | H | Y1 | H | H | H | D | D |
| LM195 | Y2 | X7 | H | H | Y2 | H | H | H | D | D |
| LM196 | Y3 | X7 | H | H | Y3 | H | H | H | D | D |
| LM197 | Y3 | X7 | D | D | Y3 | H | H | H | D | D |
| LM198 | Y3 | X7 | D | D | Y3 | D | H | H | D | D |
| LM199 | Y3 | X7 | D | D | Y3 | D | D | H | D | D |
| LM200 | Y3 | X7 | D | D | Y3 | D | D | D | D | D |
| LM201 | Y3 | Y15 | D | D | Y3 | D | D | D | D | D |
| LM202 | Y3 | Y16 | D | D | Y3 | D | D | D | D | D |
| LM203 | Y3 | Y17 | D | D | Y3 | D | D | D | D | D |
| LM204 | Y3 | Y18 | D | D | Y3 | D | D | D | D | D |
| LM205 | Y3 | Y15 | D | D | Y3 | H | X1 | H | D | D |
| LM206 | Y3 | Y15 | D | D | Y3 | D | Y3 | D | D | D |
| LM207 | Y3 | Y16 | D | D | Y3 | D | Y3 | D | D | D |
| LM208 | Y3 | Y17 | D | D | Y3 | D | Y3 | D | D | D |
| LM209 | Y3 | Y18 | D | D | Y3 | D | Y3 | D | D | D |
| LM210 | Y3 | Y15 | D | D | Y3 | H | X4 | H | D | D |
| LM211 | Y3 | Y15 | D | D | Y3 | D | Y12 | D | D | D |
| LM212 | Y3 | Y16 | D | D | Y3 | D | Y12 | D | D | D |
| LM213 | Y3 | Y17 | D | D | Y3 | D | Y12 | D | D | D |
| LM214 | Y3 | Y18 | D | D | Y3 | D | Y12 | D | D | D |
| LM215 | X2 | X7 | H | H | X2 | H | H | H | H | D |
| LM216 | X2 | X7 | H | H | X2 | H | H | H | D | H |
| LM217 | X2 | X7 | H | H | X2 | H | H | H | D | D |
| LM218 | Y4 | X7 | H | H | Y4 | H | H | H | D | D |
| LM219 | Y5 | X7 | H | H | Y5 | H | H | H | D | D |
| LM220 | Y6 | X7 | H | H | Y6 | H | H | H | D | D |
| LM221 | Y7 | X7 | H | H | Y7 | H | H | H | D | D |
| LM222 | Y8 | X7 | H | H | Y8 | H | H | H | D | D |
| LM223 | Y9 | X7 | H | H | Y9 | H | H | H | D | D |
| LM224 | Y10 | X7 | H | H | Y10 | H | H | H | D | D |
| LM225 | Y10 | X7 | D | D | Y10 | H | H | H | D | D |
| LM226 | Y10 | X7 | D | D | Y10 | D | H | H | D | D |
| LM227 | Y10 | X7 | D | D | Y10 | D | D | H | D | D |
| LM228 | Y10 | X7 | D | D | Y10 | D | D | D | D | D |
| LM229 | Y10 | X8 | D | D | Y10 | D | D | D | D | D |
| LM230 | Y10 | Y16 | D | D | Y10 | D | D | D | D | D |
| LM231 | Y10 | Y17 | D | D | Y10 | D | D | D | D | D |
| LM232 | Y10 | Y18 | D | D | Y10 | D | D | D | D | D |
| LM233 | Y10 | Y15 | D | D | Y10 | D | D | D | D | D |
| LM234 | Y10 | Y15 | D | D | Y10 | H | X1 | H | D | D |
| LM235 | Y10 | Y15 | D | D | Y10 | D | Y3 | D | D | D |
| LM236 | Y10 | Y16 | D | D | Y10 | D | Y3 | D | D | D |
| LM237 | Y10 | Y17 | D | D | Y10 | D | Y3 | D | D | D |
| LM238 | Y10 | Y18 | D | D | Y10 | D | Y3 | D | D | D |
| LM239 | Y10 | Y15 | D | D | Y10 | H | X4 | H | D | D |
| LM240 | Y10 | Y15 | D | D | Y10 | D | Y12 | D | D | D |
| LM241 | Y10 | Y16 | D | D | Y10 | D | Y12 | D | D | D |
| LM242 | Y10 | Y17 | D | D | Y10 | D | Y12 | D | D | D |
| LM243 | Y10 | Y18 | D | D | Y10 | D | Y12 | D | D | D |

TABLE 15
Formula 1-2

| Ligand | R₁₁ | X₁₁ | R₁₀₁ | R₁₀₂ | R₁₀₃ | R₁₀₄ | R₁₄ | R₁₅ | R₁₆ | R₁₇ | R₁₈ | R₁₉ | R₂₀ |
|---|---|---|---|---|---|---|---|---|---|---|---|---|---|
| LFM1 | Y10 | N—Ph | D | D | D | D | D | Y10 | D | D | D | D | D |
| LFM2 | Y10 | S | D | D | D | D | D | Y10 | D | D | D | D | D |
| LFM3 | Y10 | O | D | D | D | D | D | Y10 | D | D | D | D | D |
| LFM4 | Y3 | O | D | D | D | D | D | Y3 | D | D | D | D | D |
| LFM5 | Y10 | O | D | D | D | D | D | Y10 | D | D | D | D | D |
| LFM6 | Y10 | O | D | D | D | D | D | Y10 | D | Y3 | D | D | D |
| LFM7 | Y10 | O | D | D | D | D | D | Y10 | D | Y12 | D | D | D |

TABLE 16
Formula 1-3

| Ligand | R₁₁ | X₁₁ | R₁₀₁ | R₁₀₂ | R₁₀₃ | R₁₀₄ | R₁₄ | R₁₅ | R₁₆ | R₁₇ | R₁₈ | R₁₉ | R₂₀ |
|---|---|---|---|---|---|---|---|---|---|---|---|---|---|
| LFP1 | Y10 | N—Ph | D | D | D | D | D | Y10 | D | D | D | D | D |
| LFP2 | Y10 | S | D | D | D | D | D | Y10 | D | D | D | D | D |
| LFP3 | Y10 | O | D | D | D | D | D | Y10 | D | D | D | D | D |
| LFP4 | Y3 | O | D | D | D | D | D | Y3 | D | D | D | D | D |
| LFP5 | Y10 | O | D | D | D | D | D | Y10 | D | D | D | D | D |
| LFP6 | Y10 | O | D | D | D | D | D | Y10 | D | Y3 | D | D | D |
| LFP7 | Y10 | O | D | D | D | D | D | Y10 | D | Y12 | D | D | D |

In Tables 14 to 16, X1 to X10 and Y1 to Y18 may be as follows, and “Ph” represents a phenyl group:

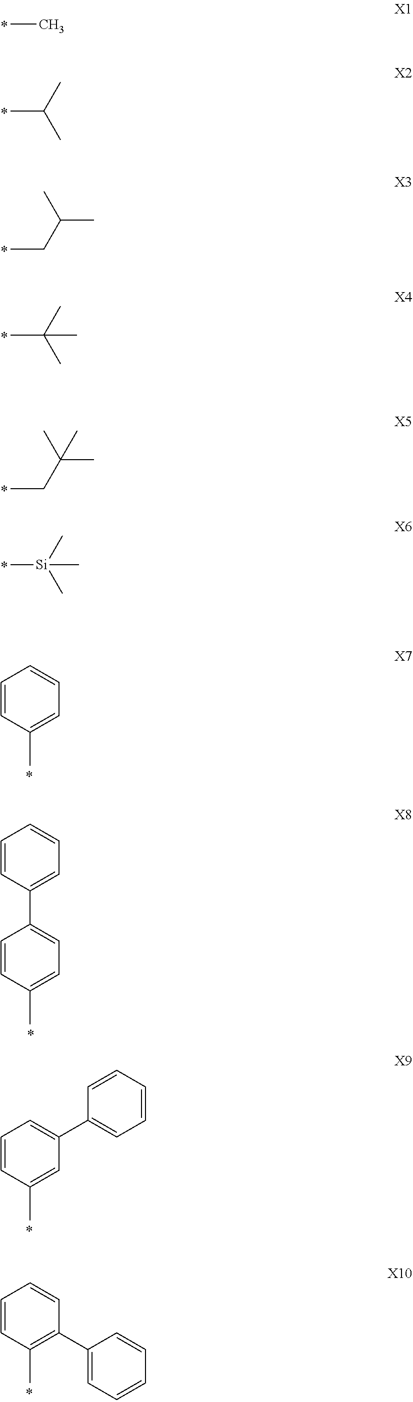

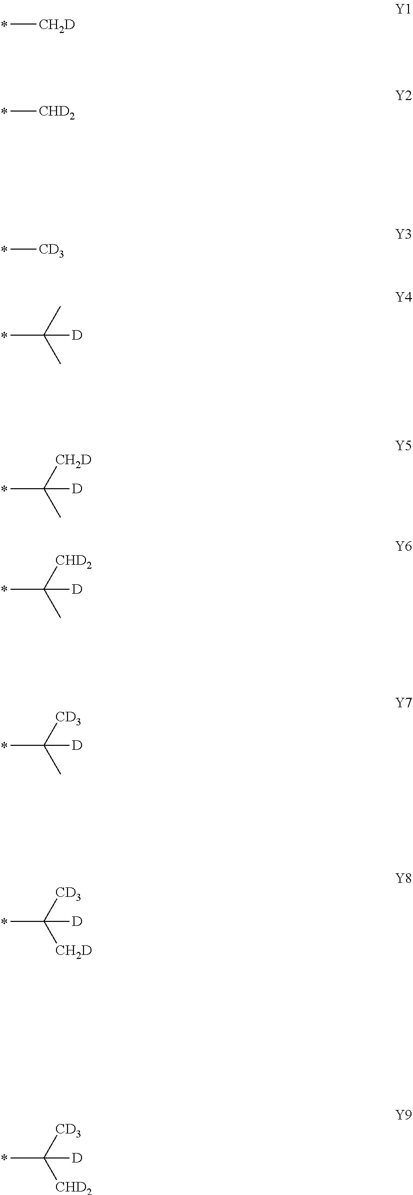

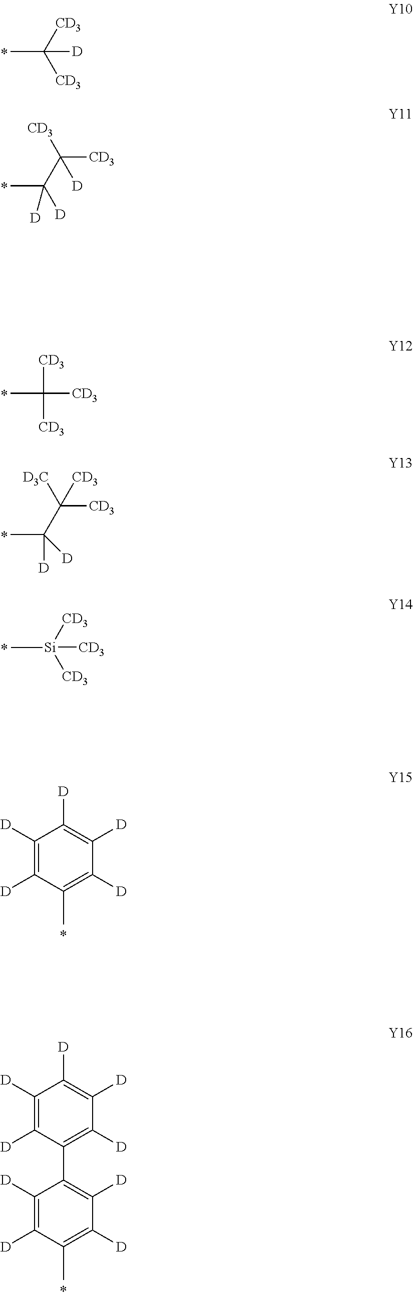

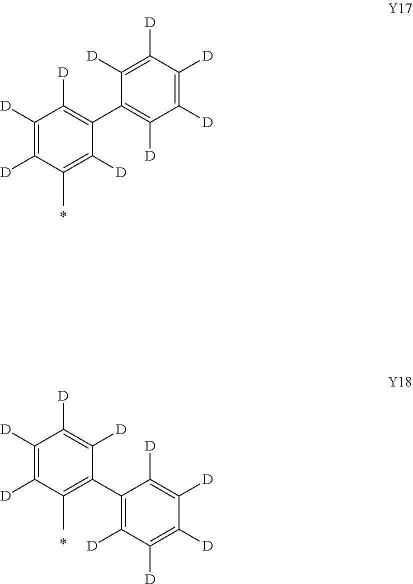 [SMILES: [2H]c1c([2H])c([2H])c(-c2c([2H])c([2H])c([2H])c(C)c2[2H])c([2H])c1[2H] ; [2H]c1c([2H])c([2H])c(-c2c([2H])c([2H])c([2H])c([2H])c2C)c([2H])c1[2H]]

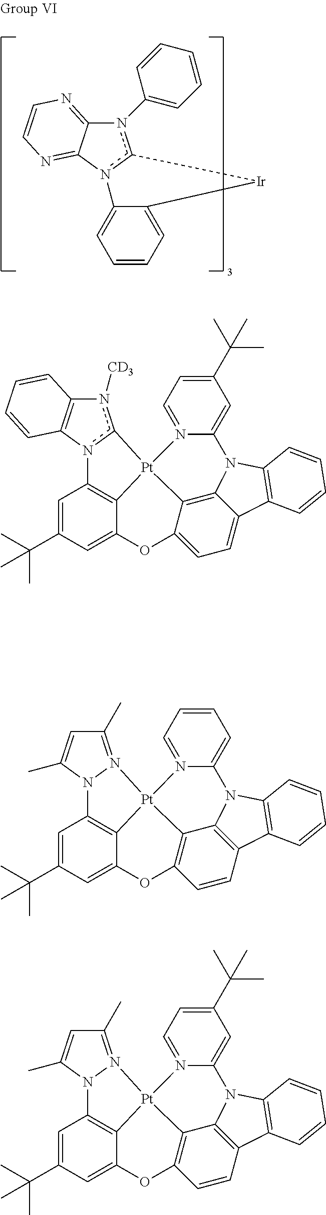

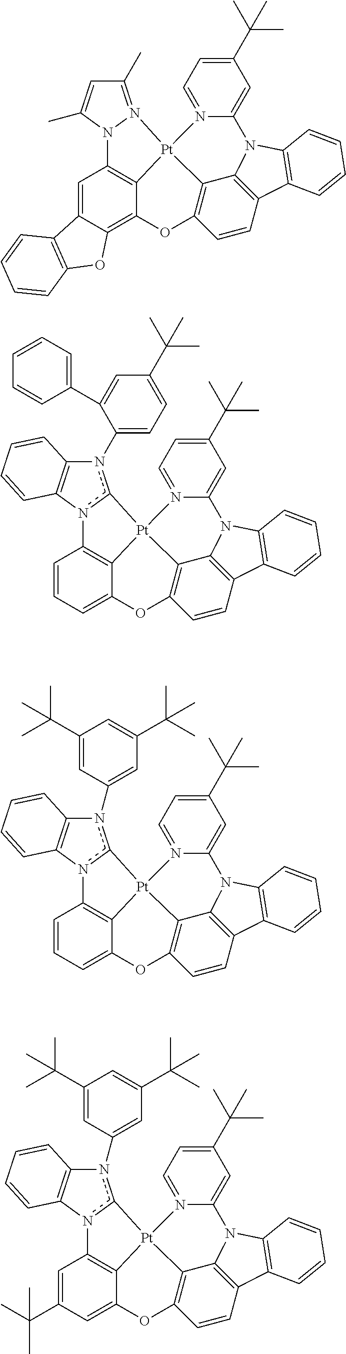

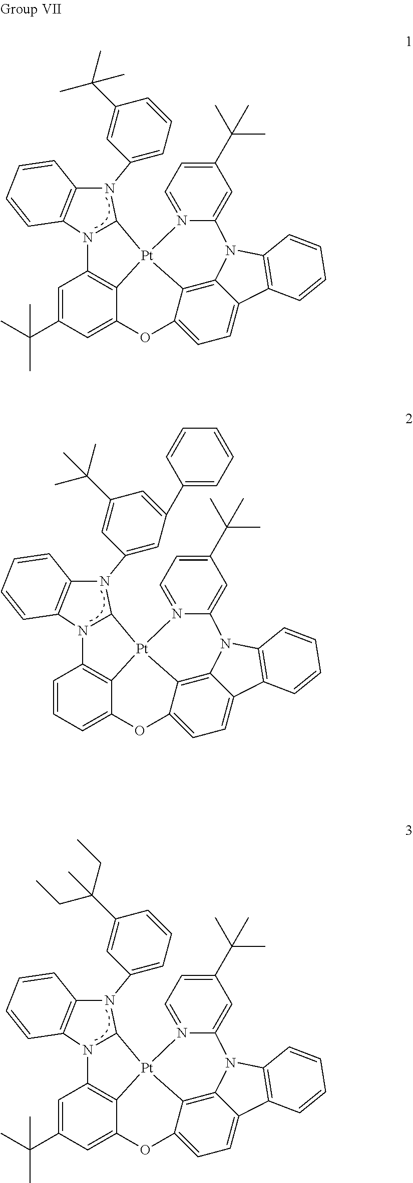

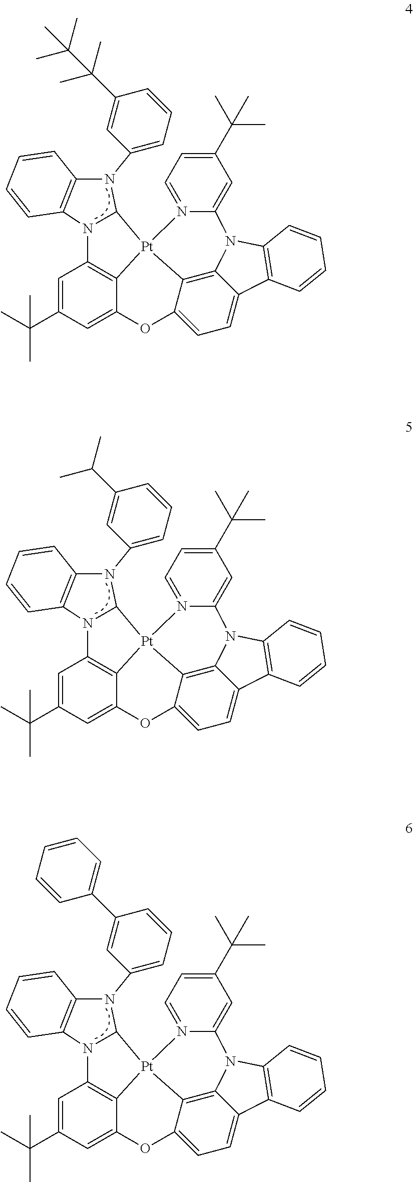

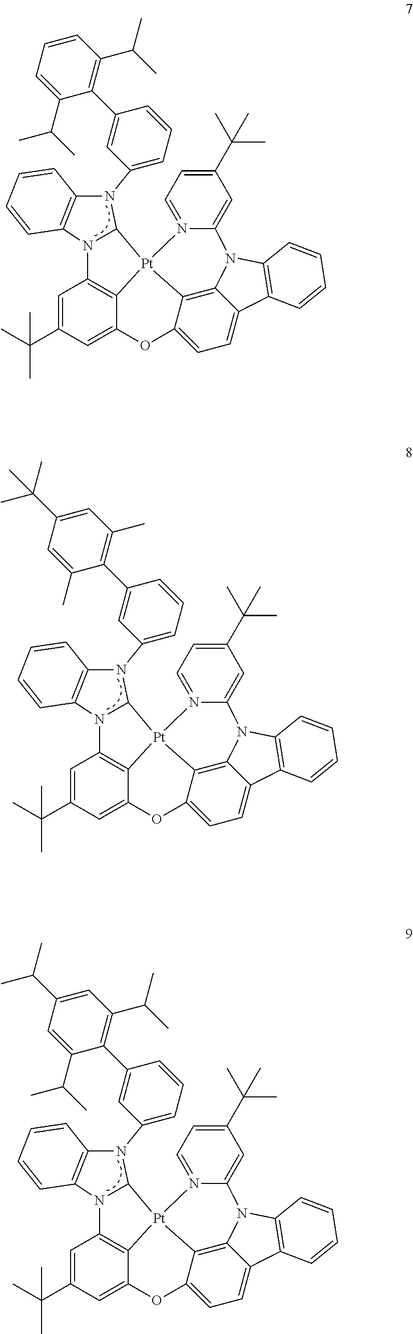

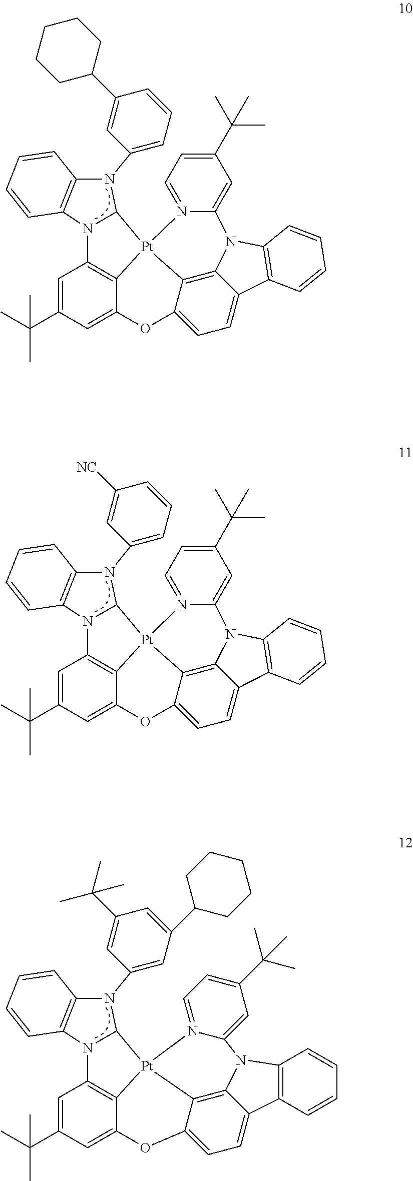

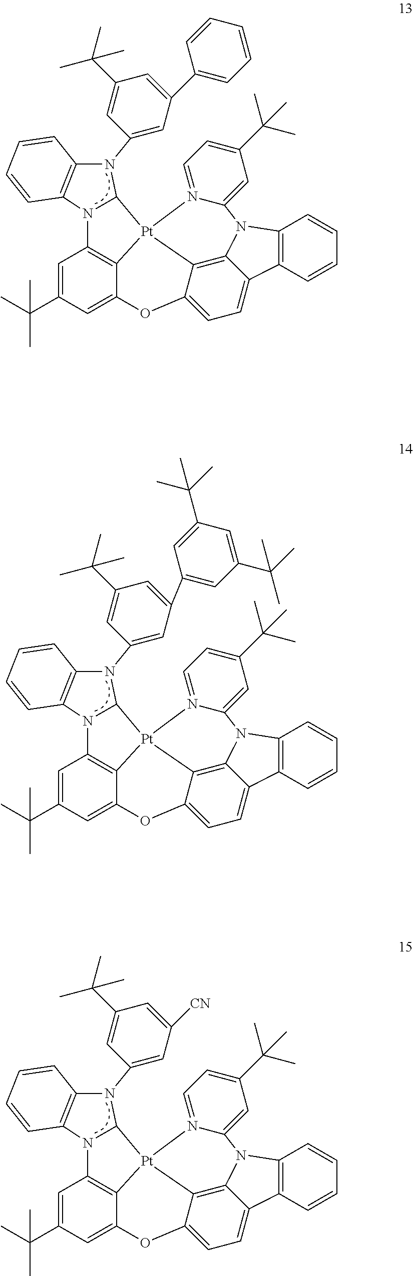

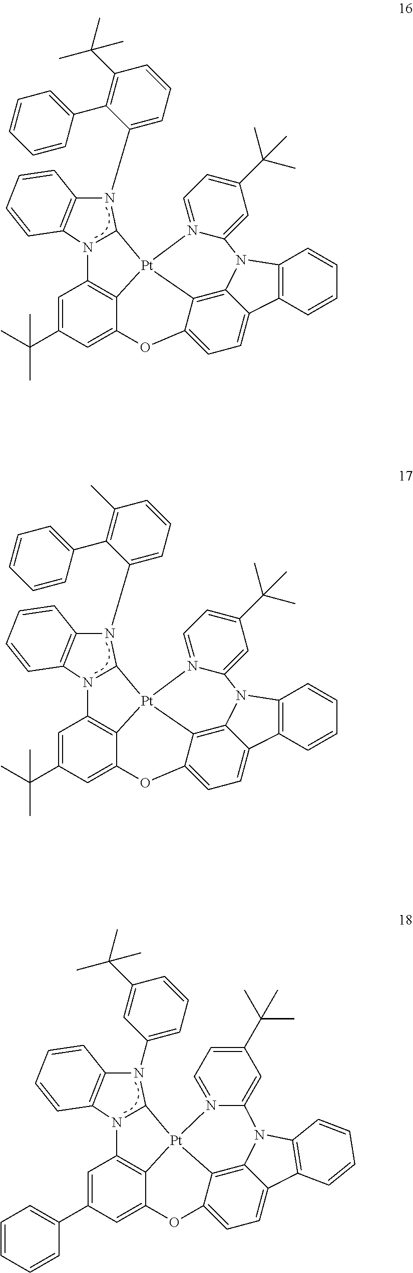

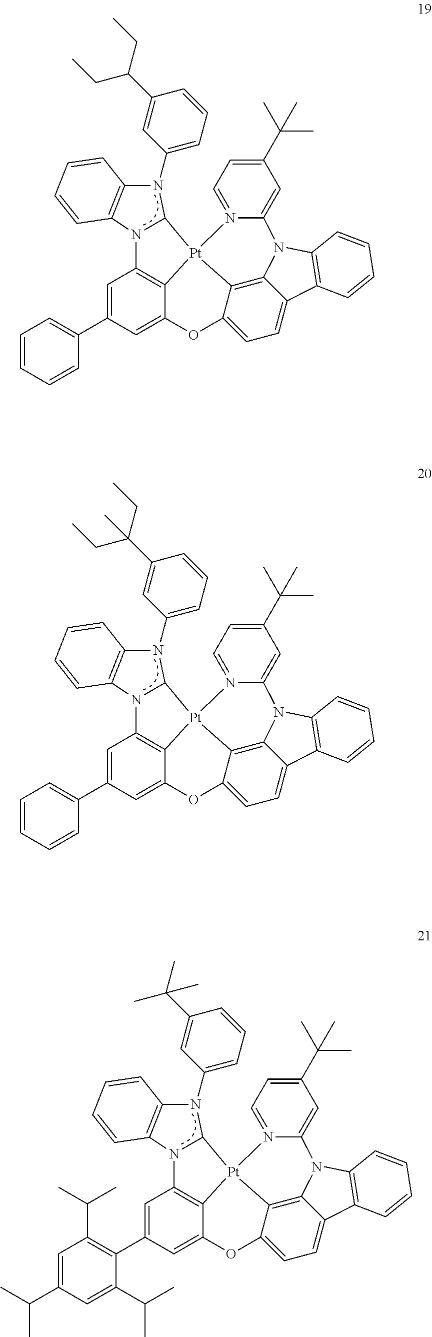

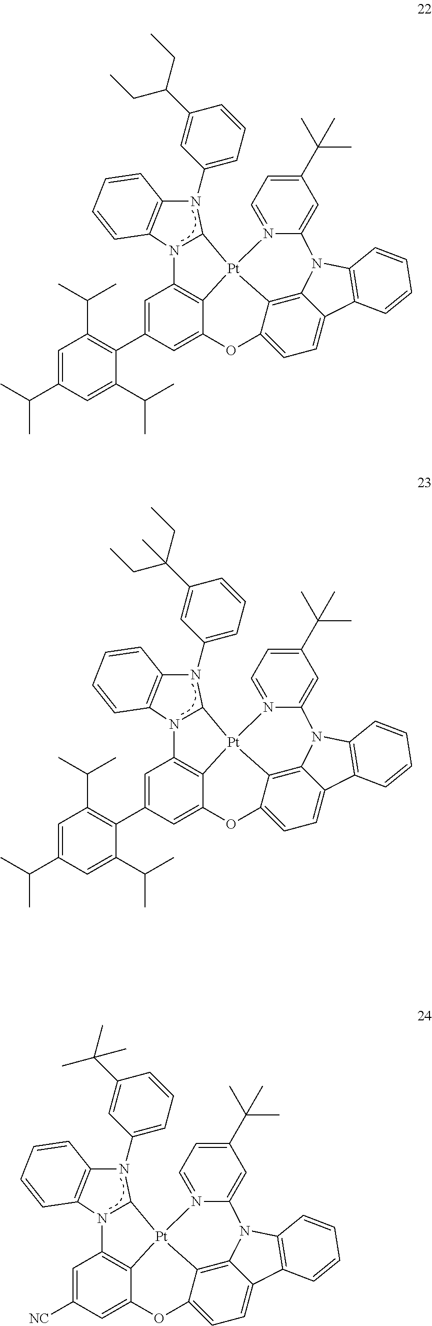

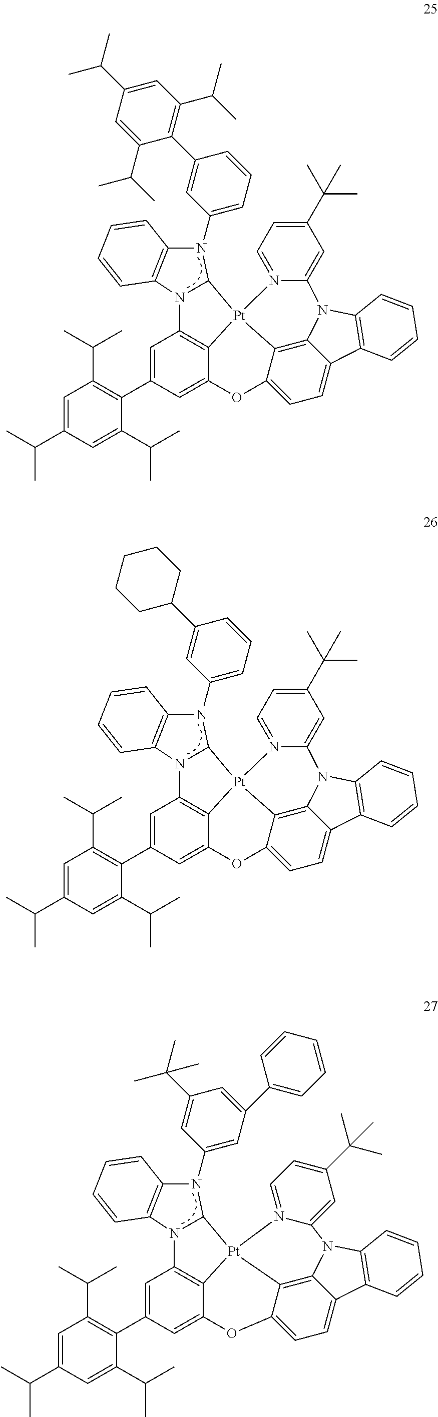

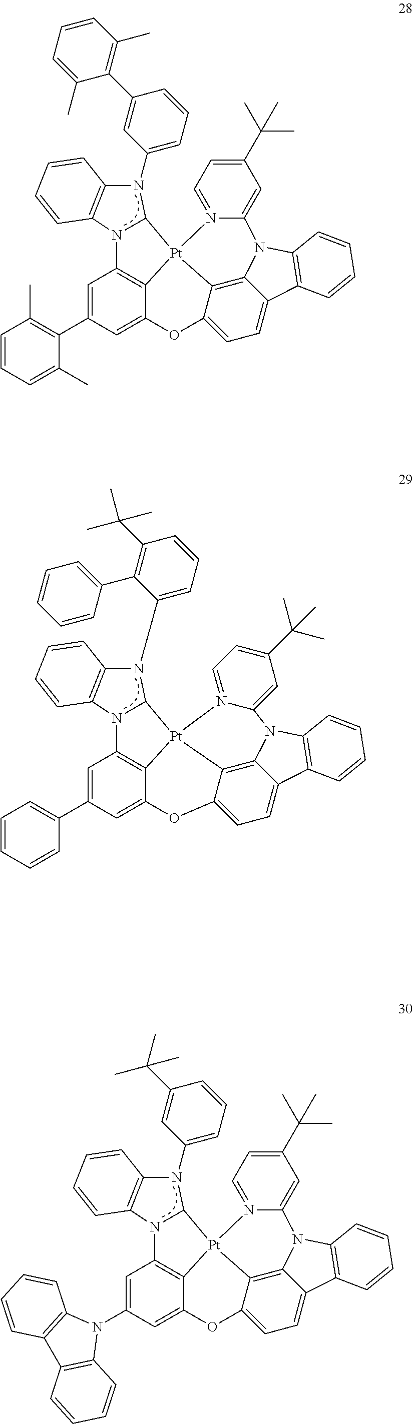

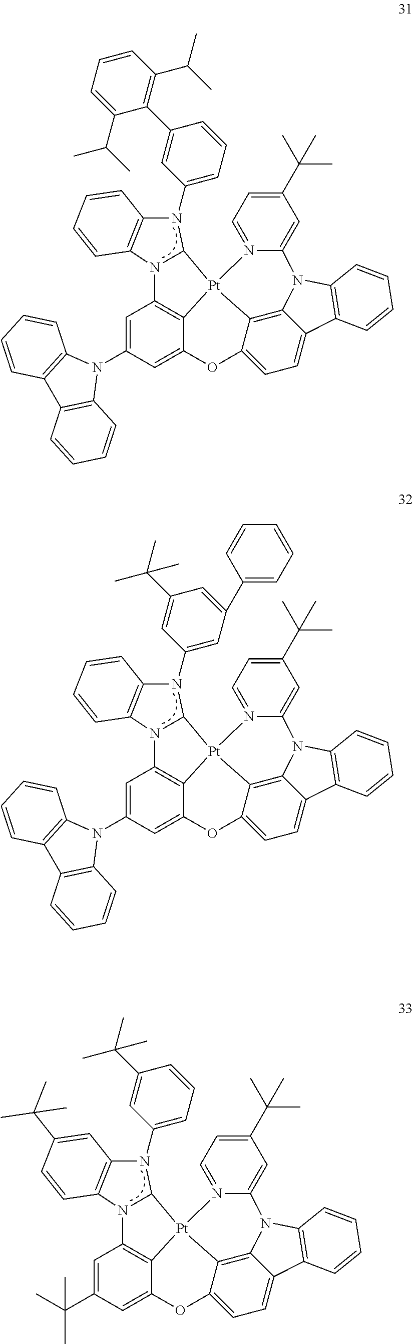

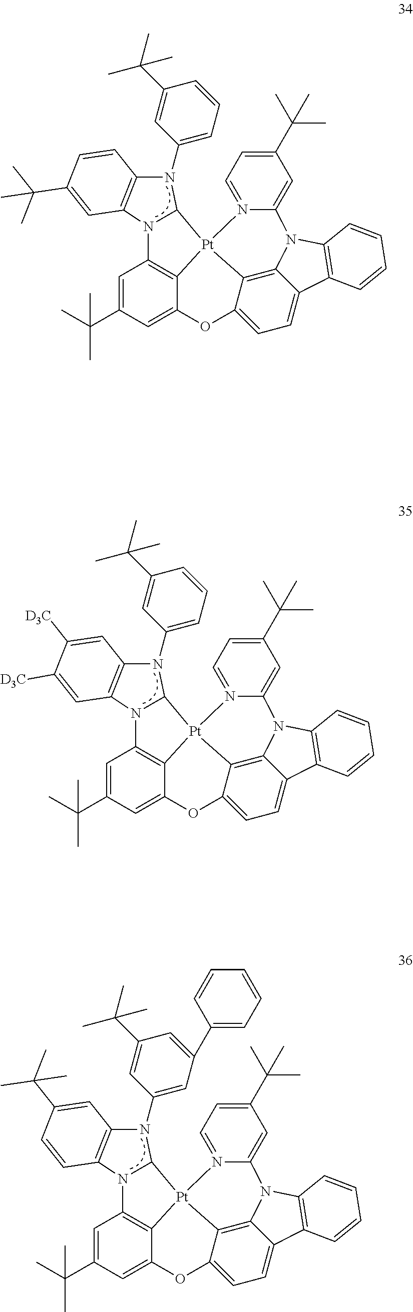

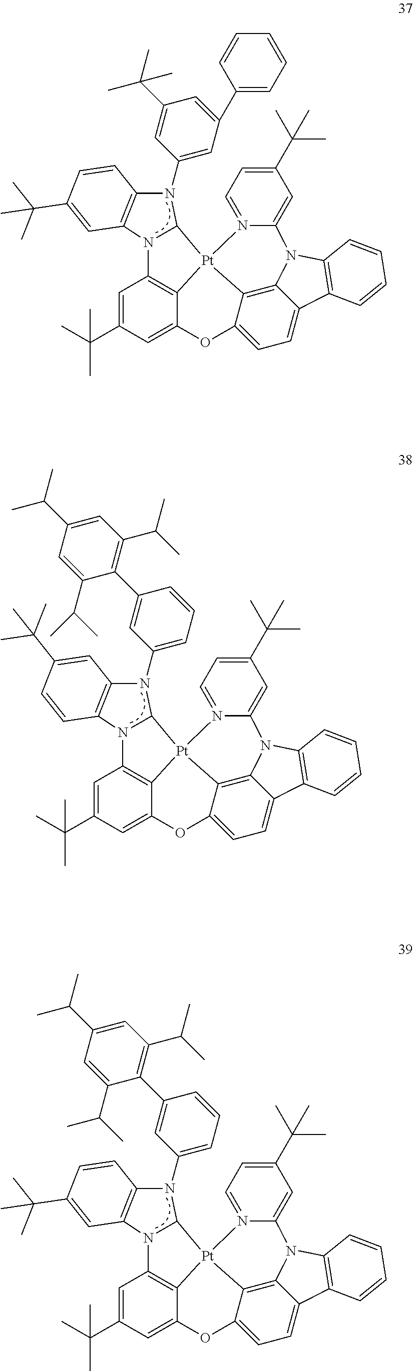

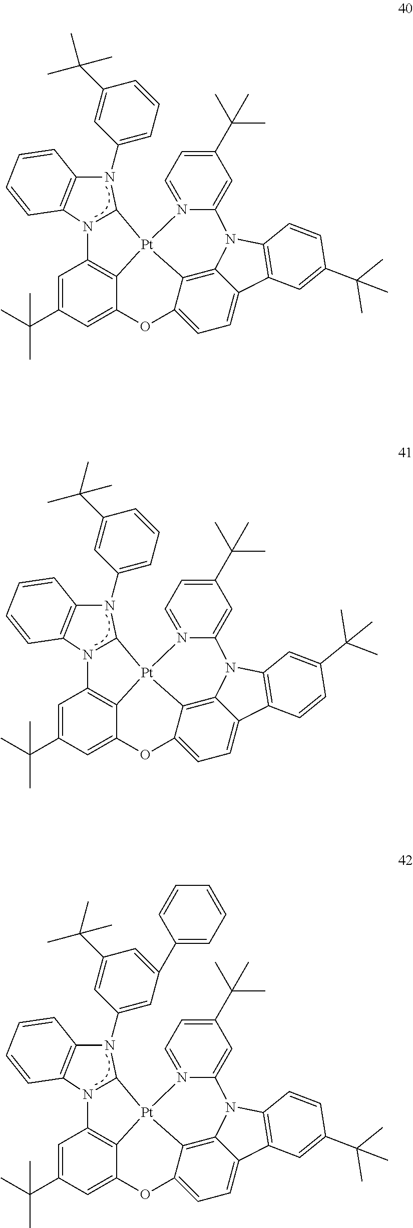

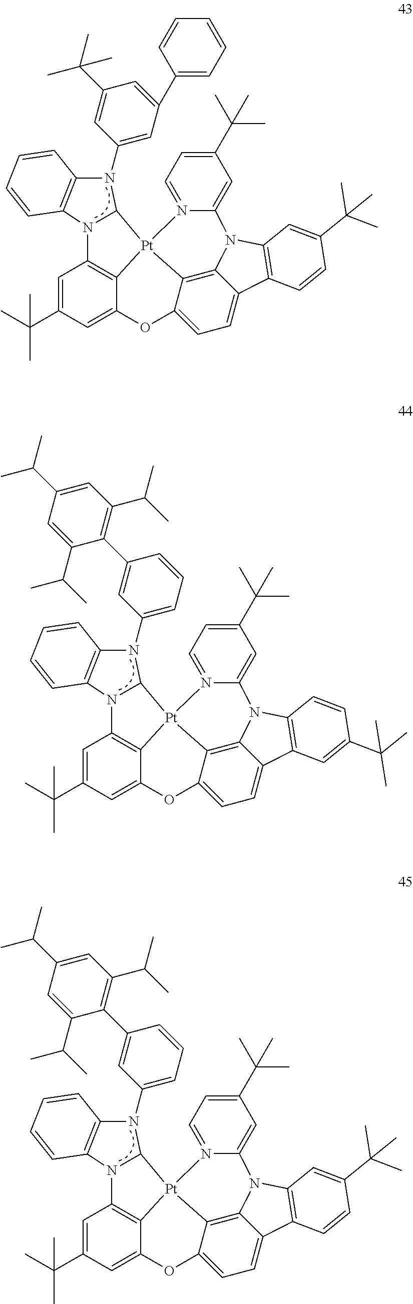

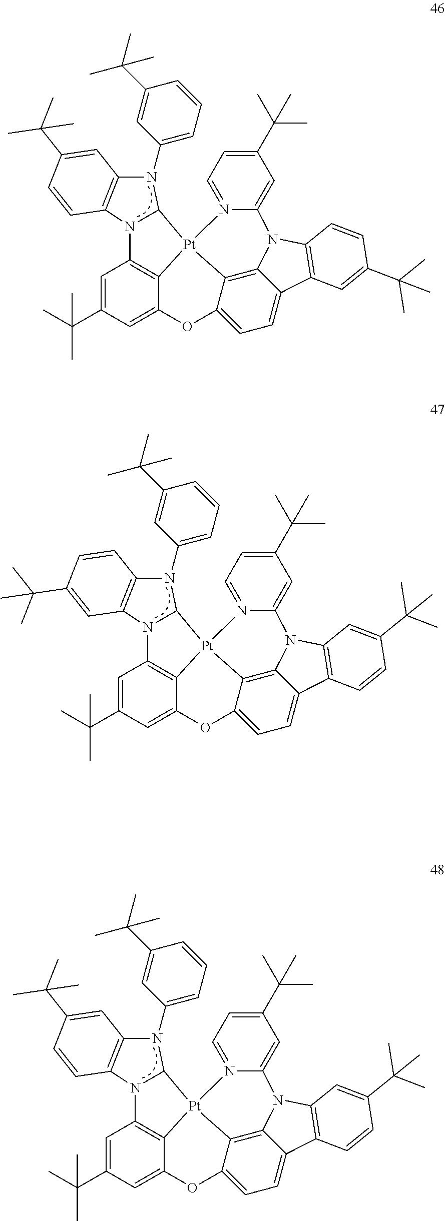

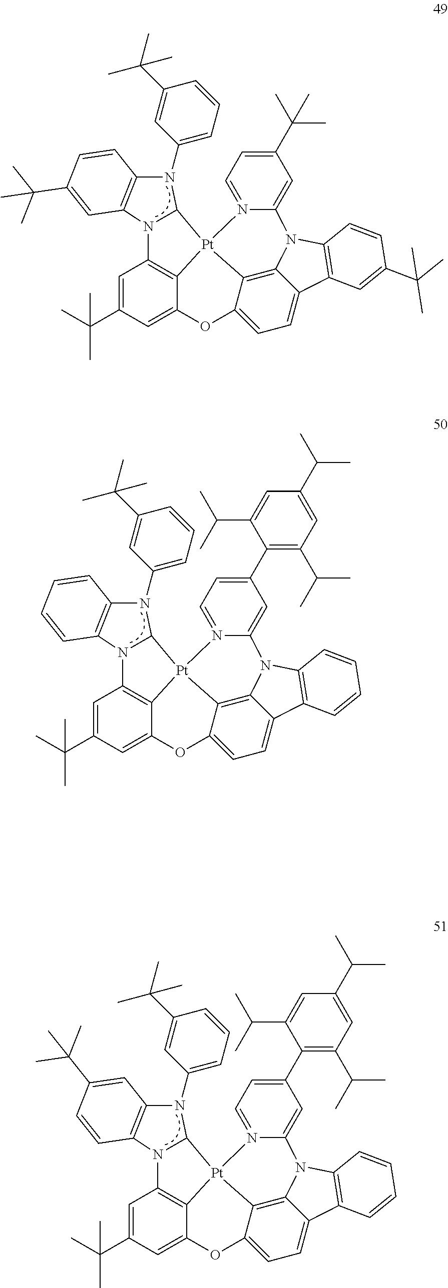

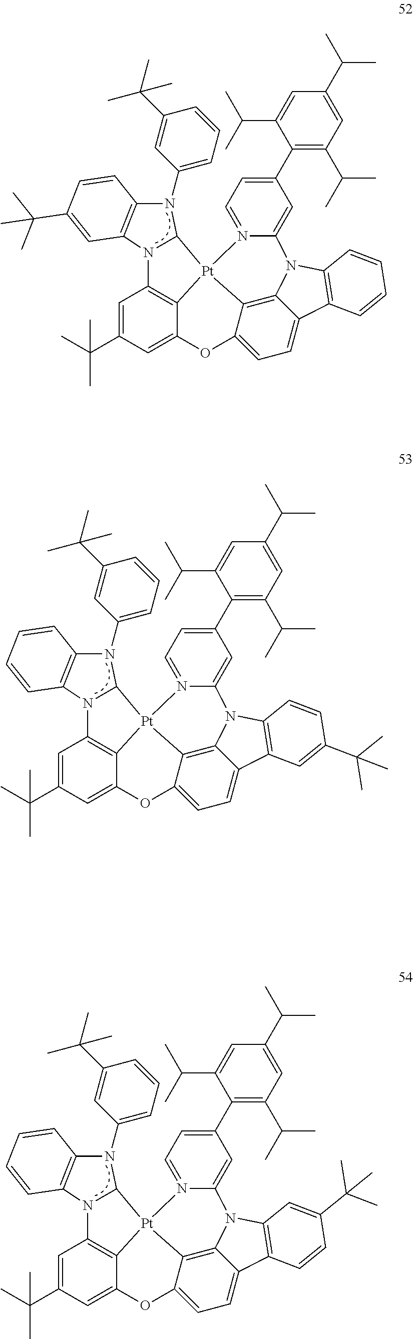

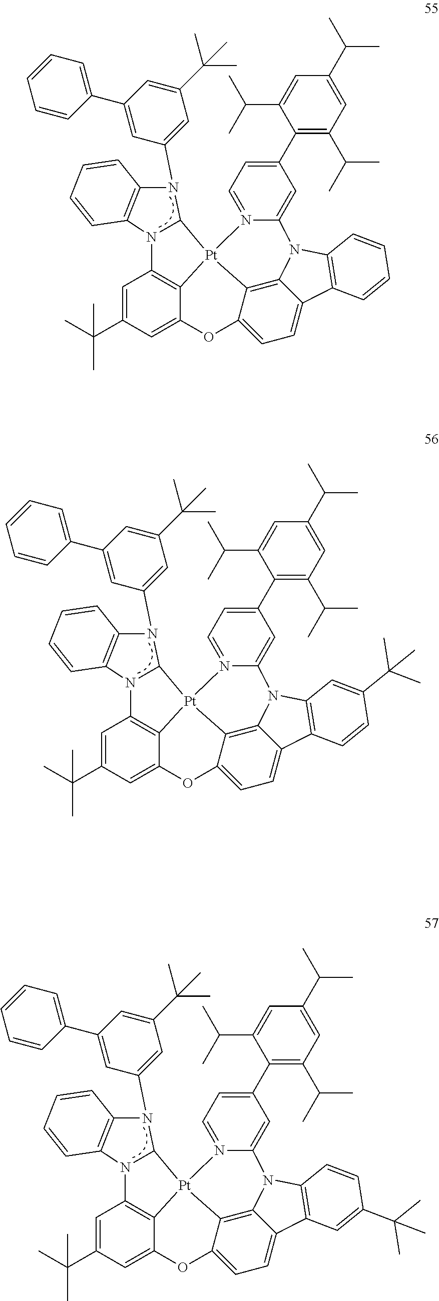

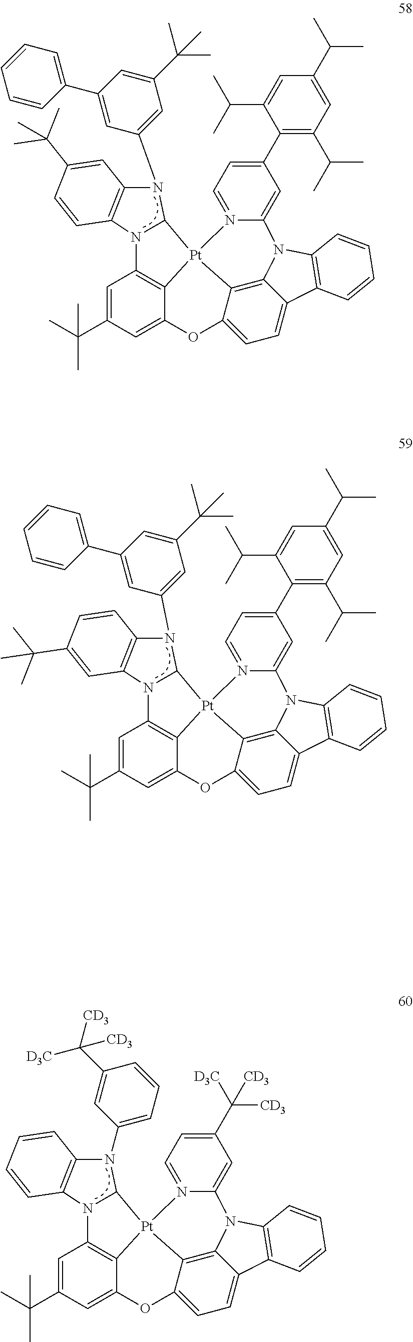

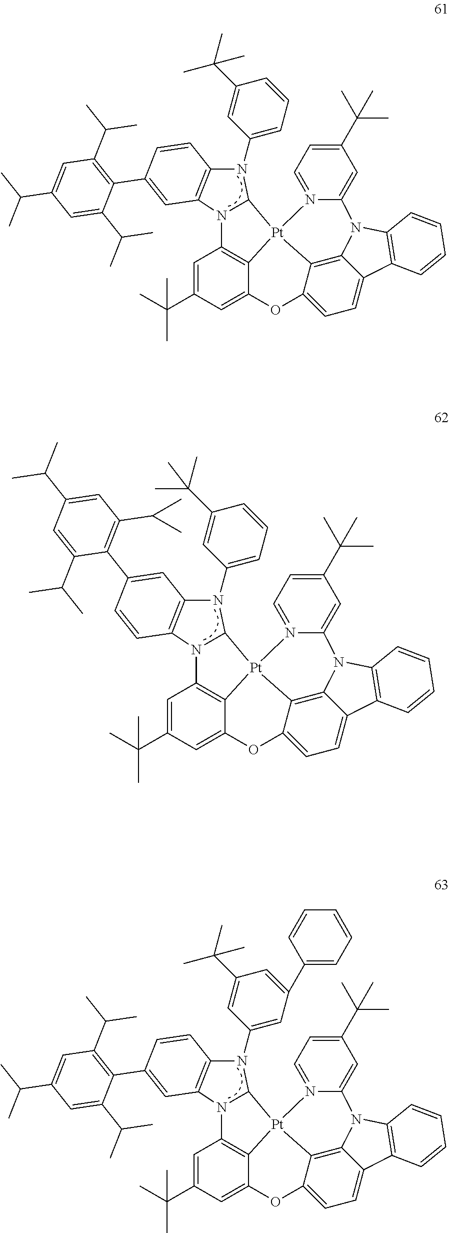

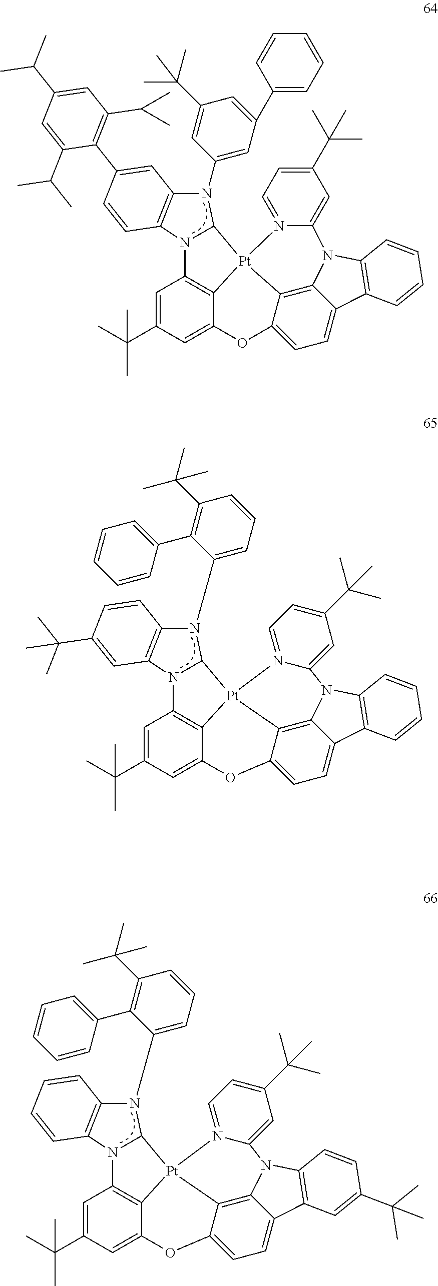

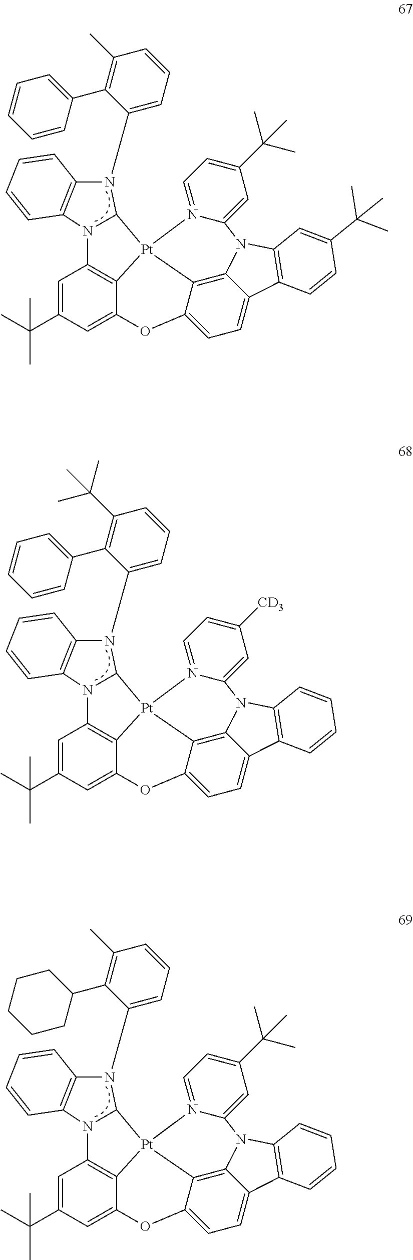

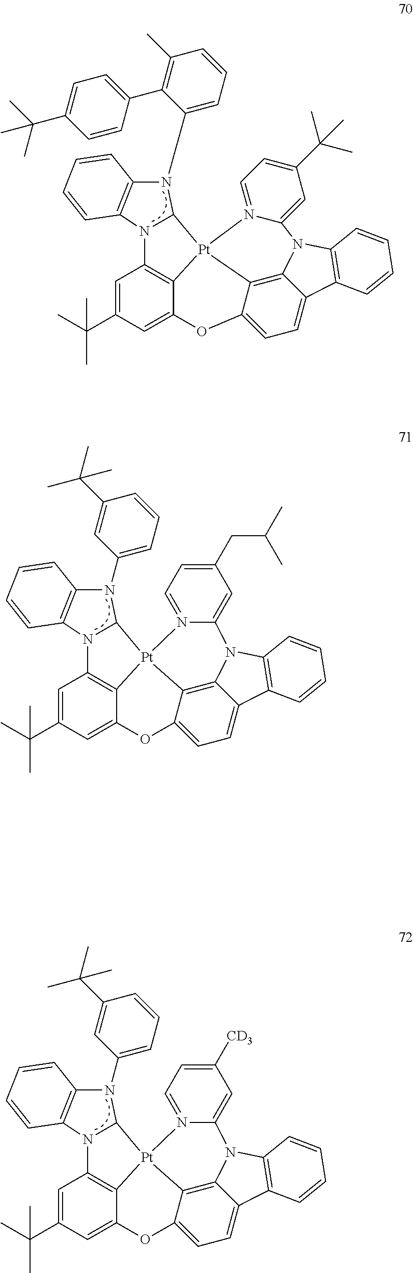

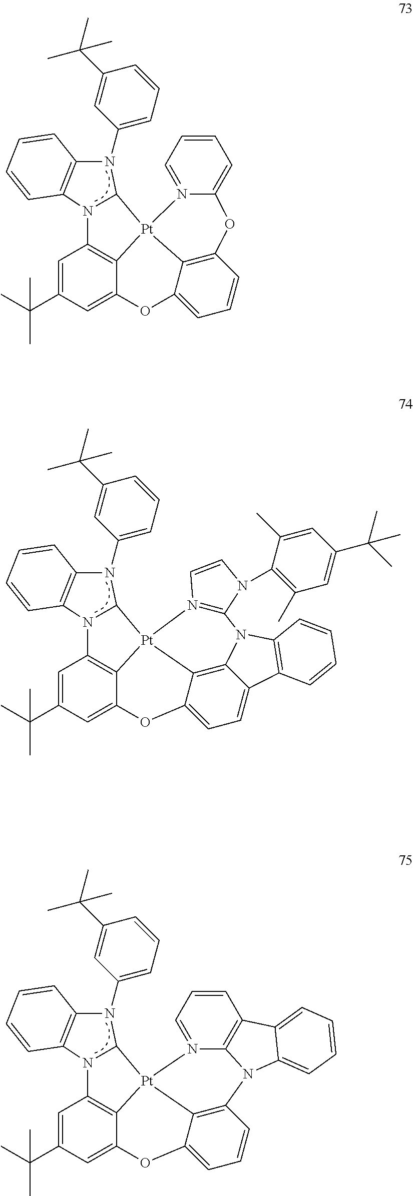

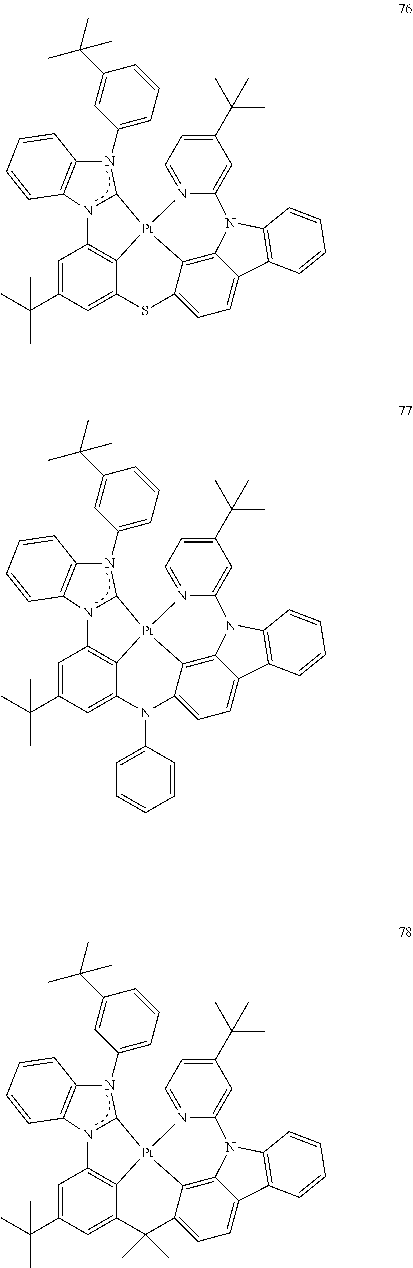

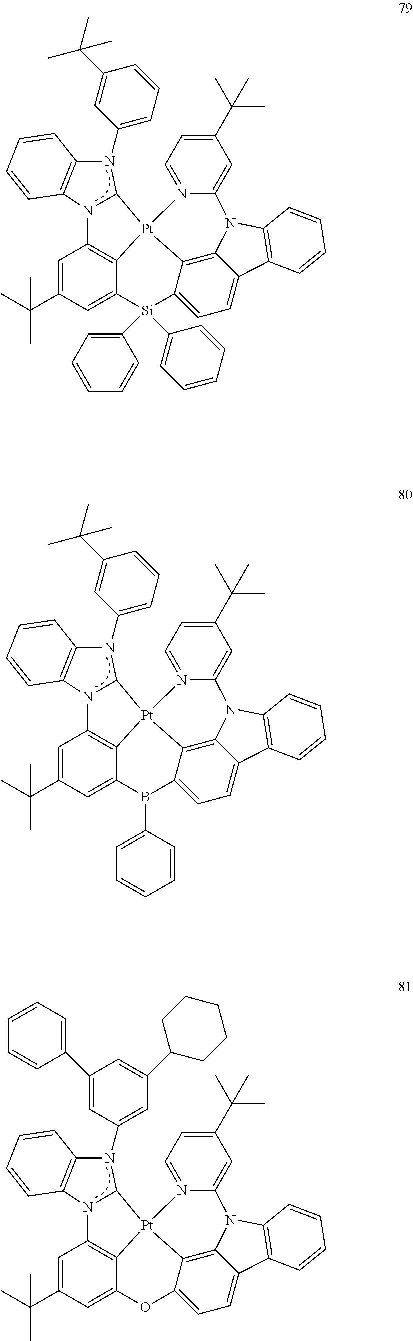

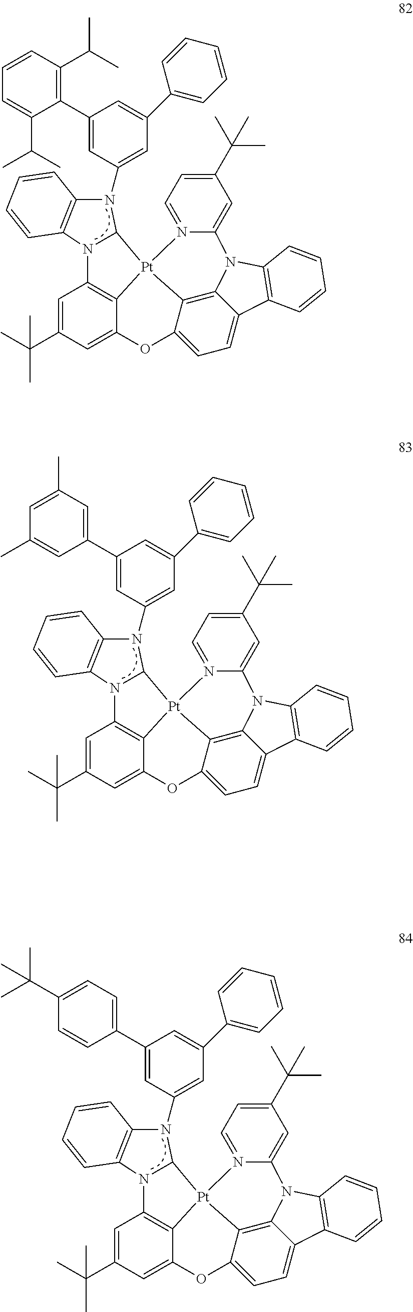

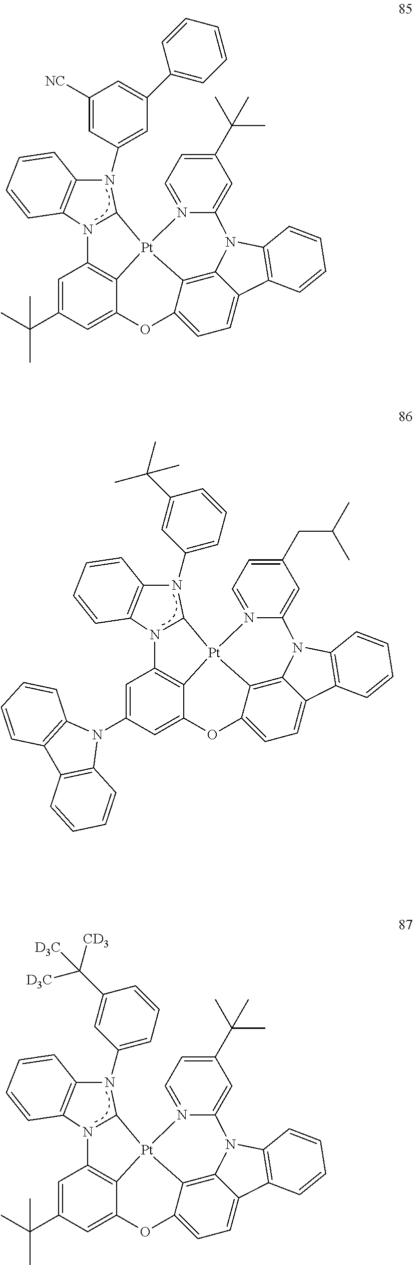

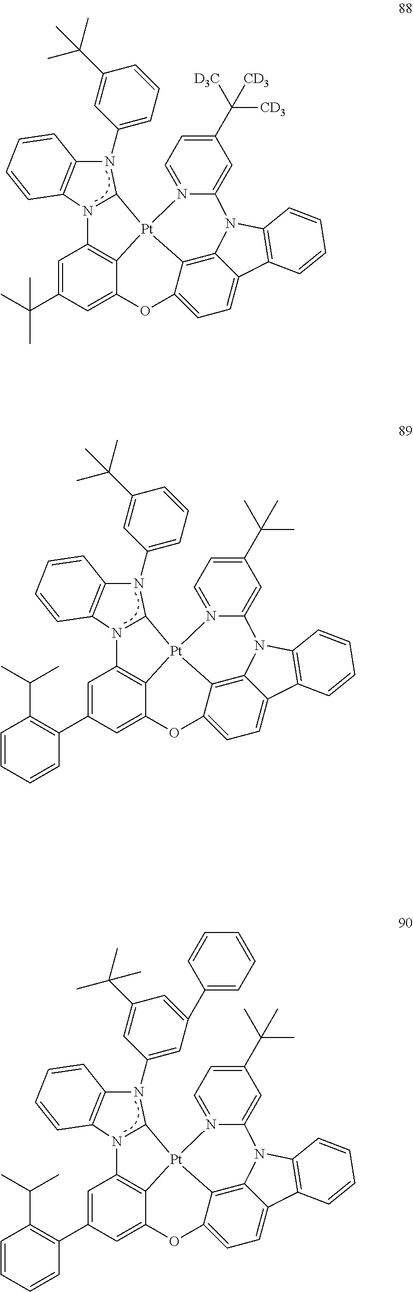

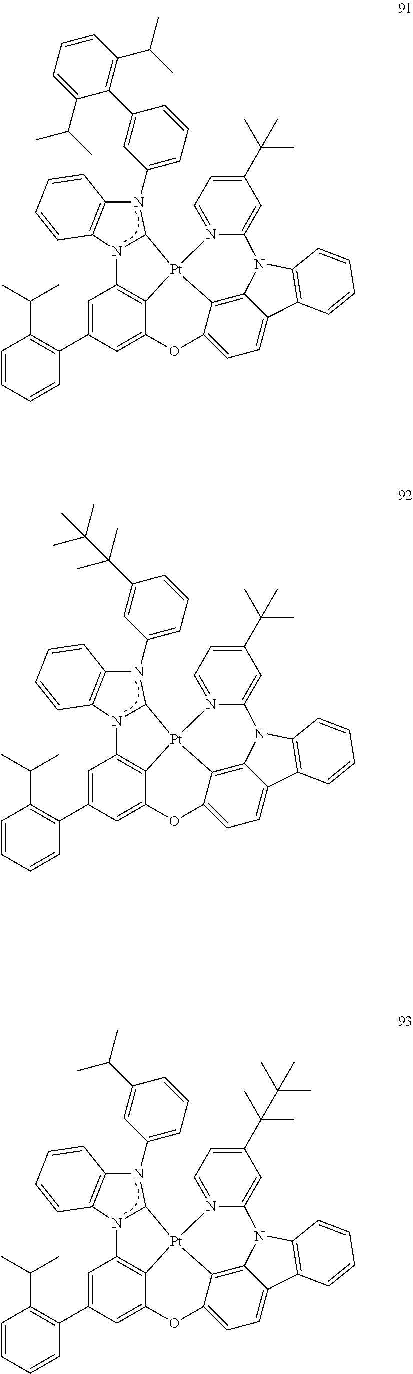

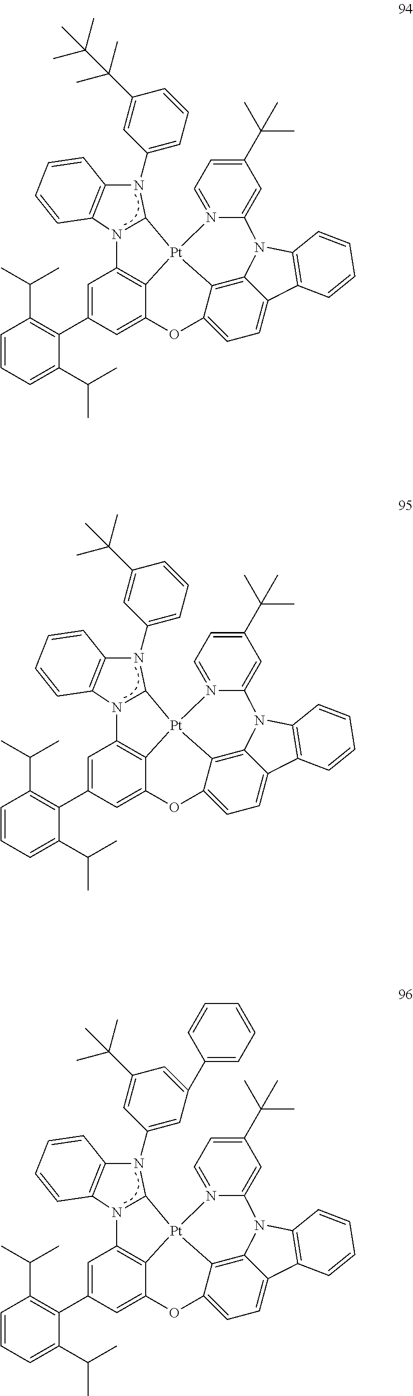

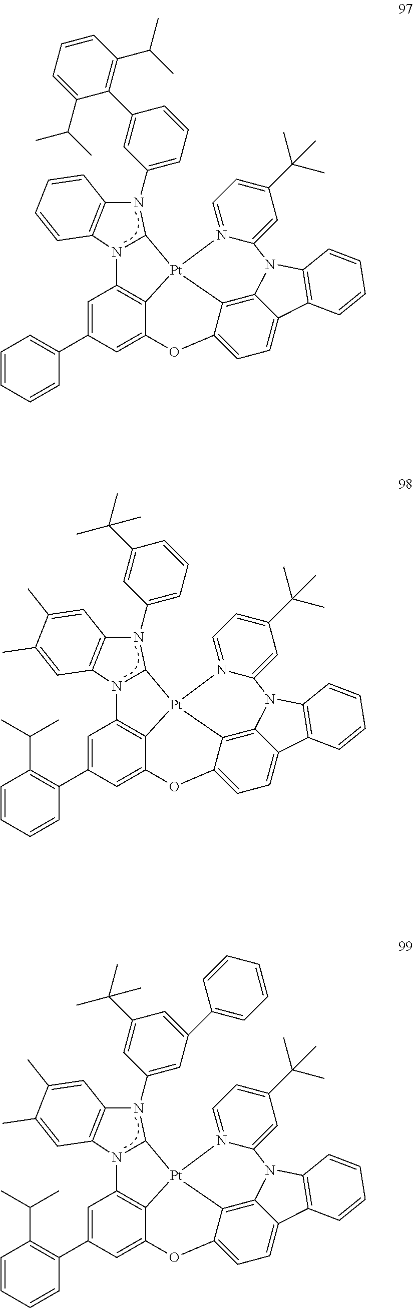

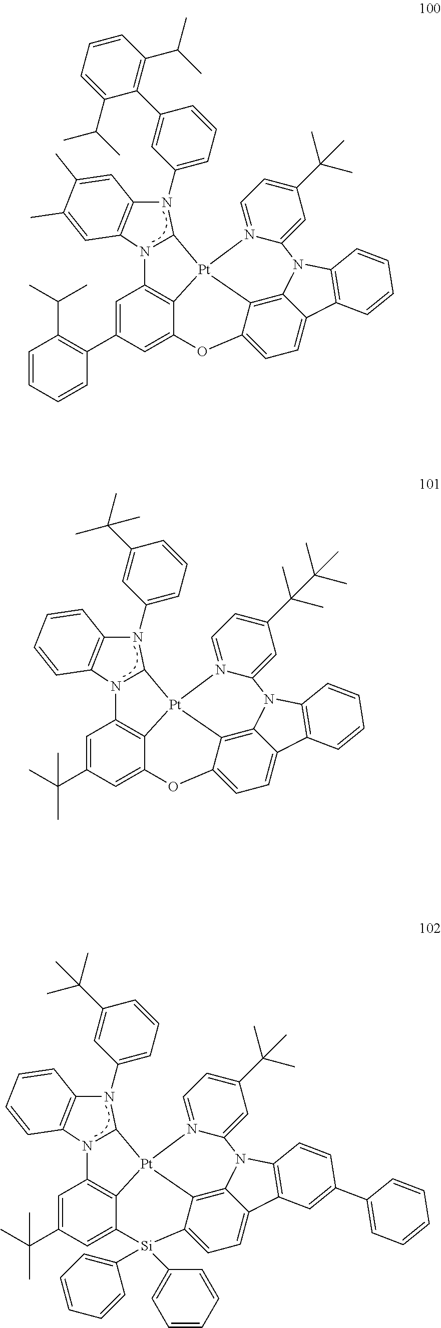

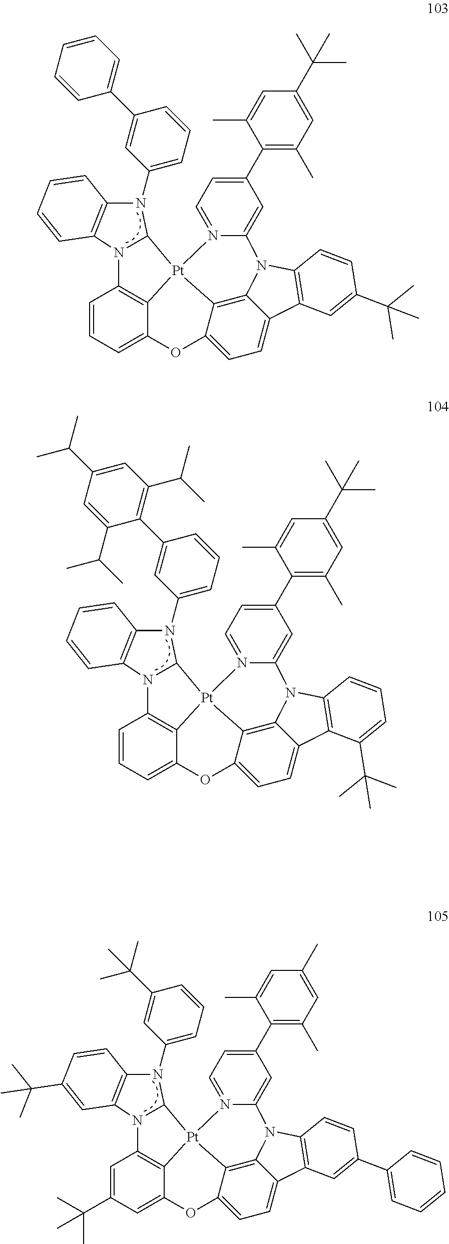

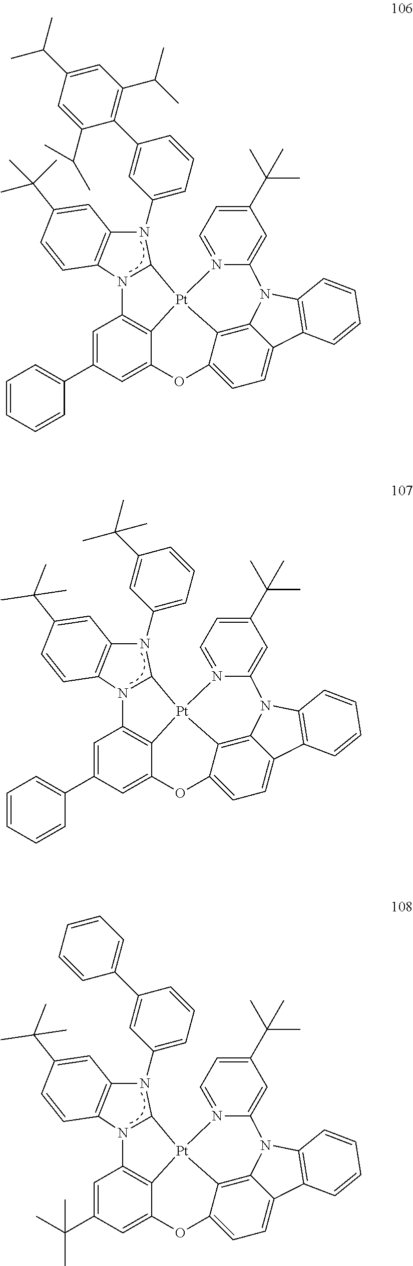

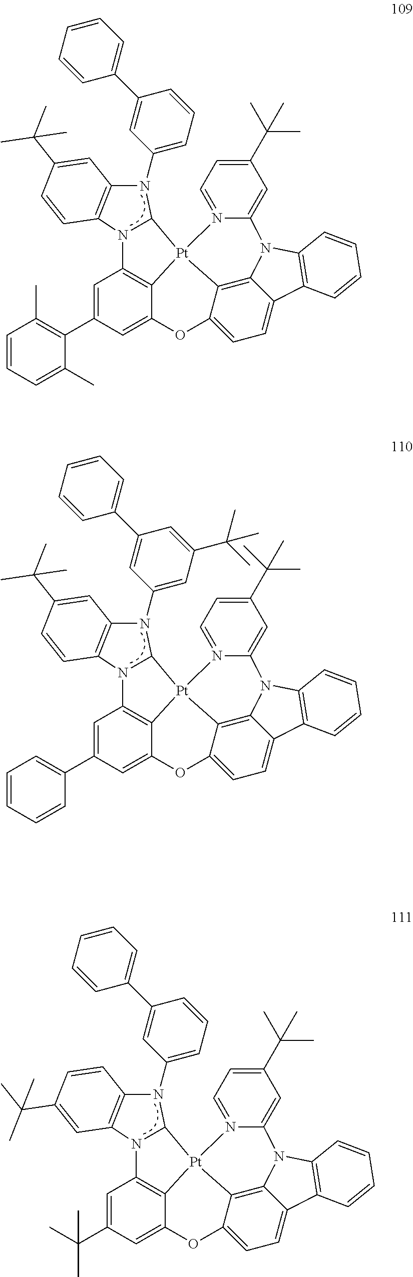

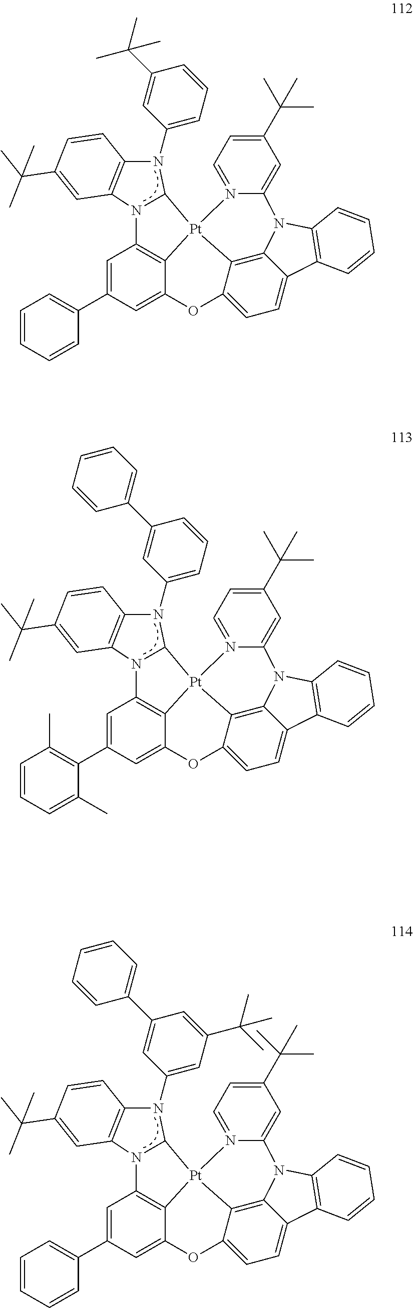

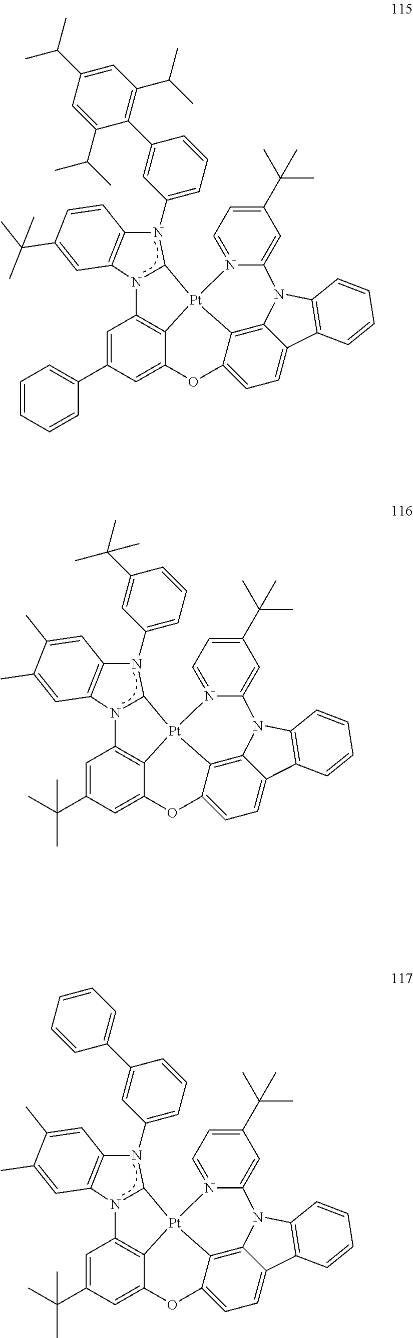

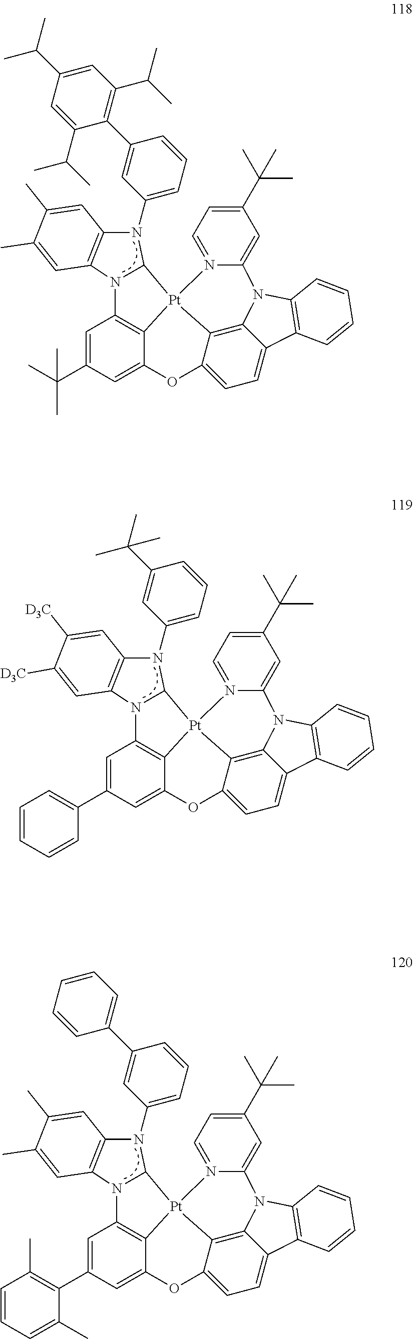

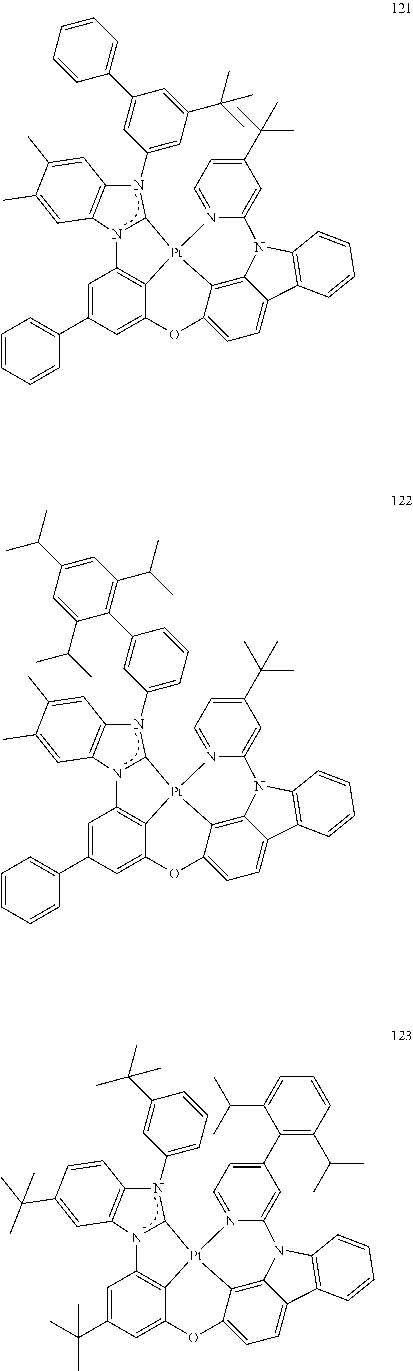

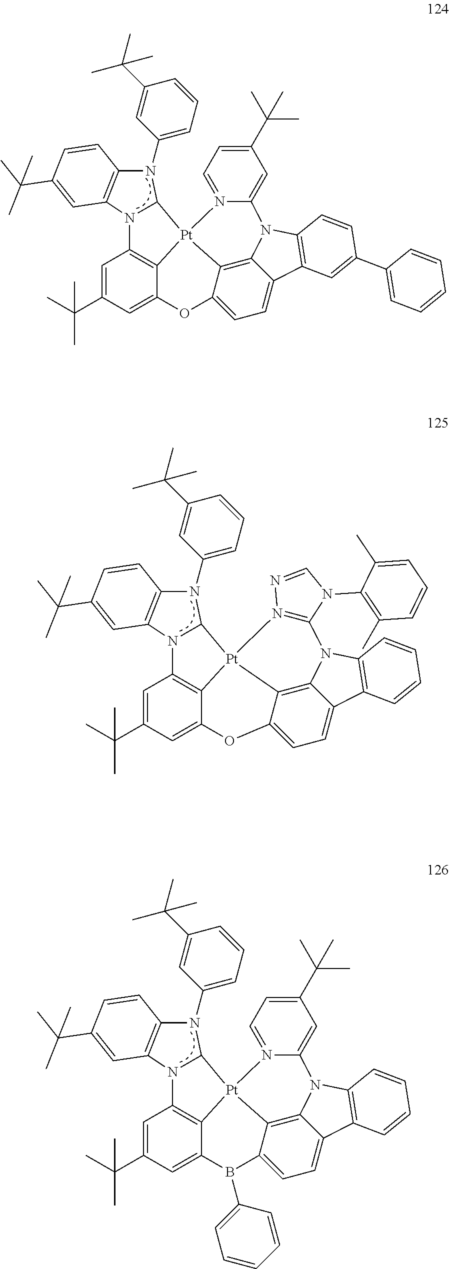

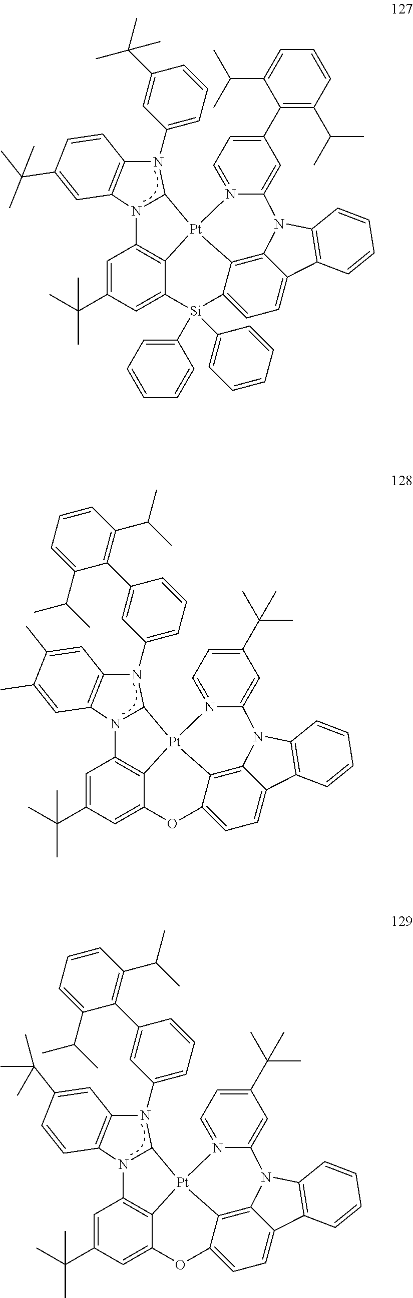

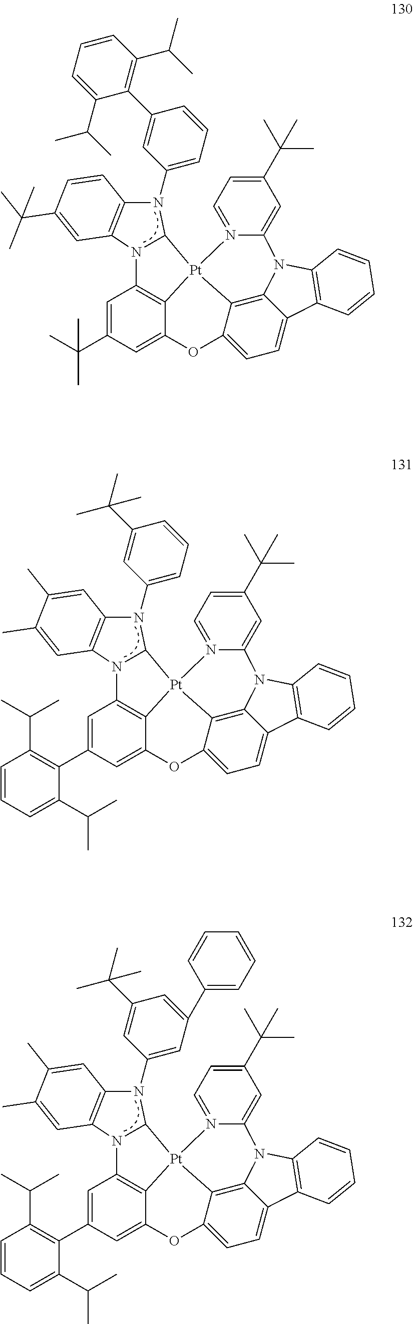

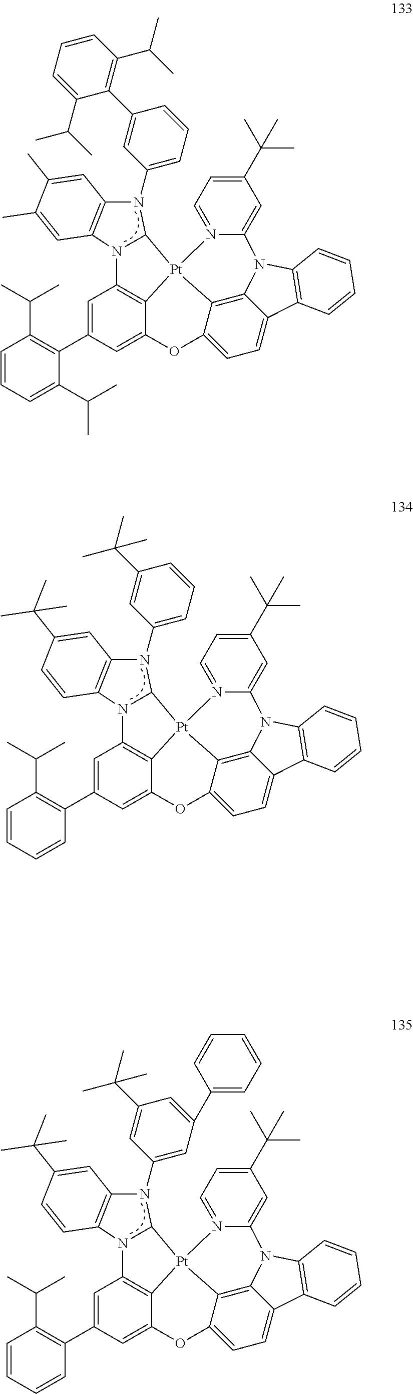

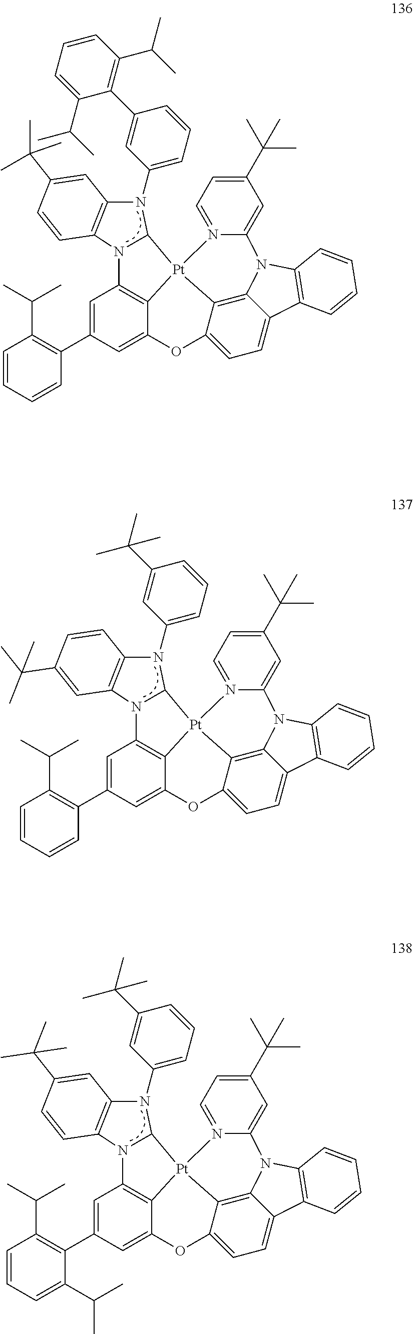

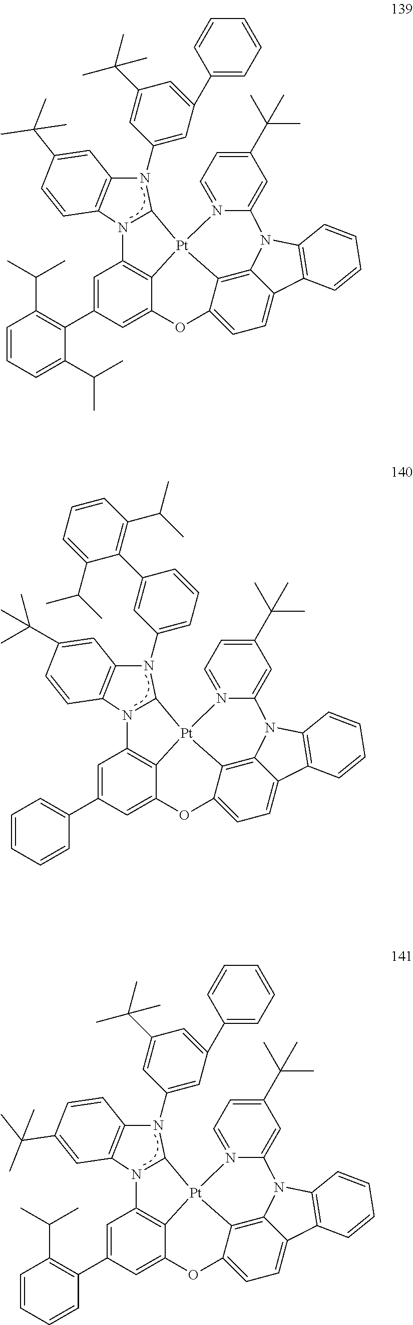

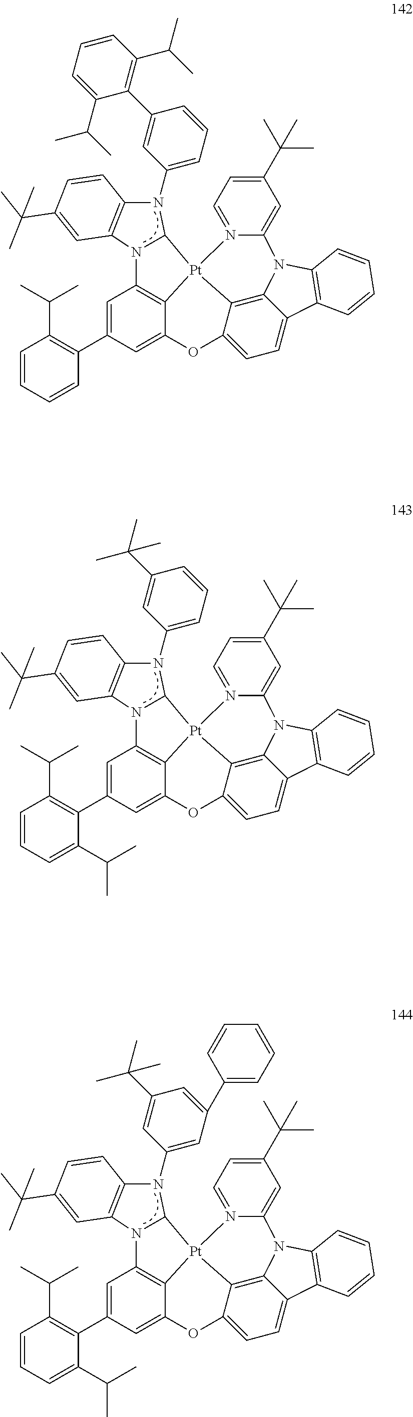

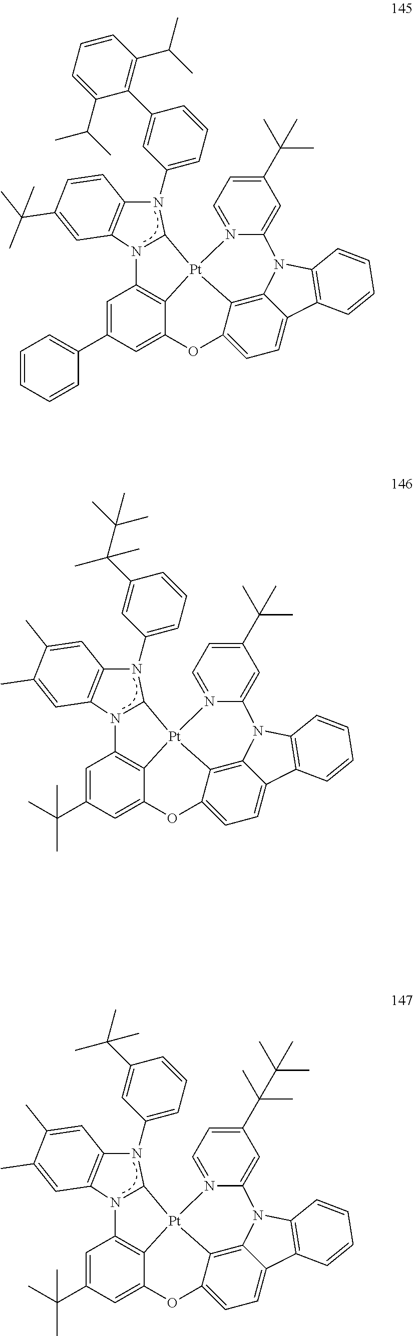

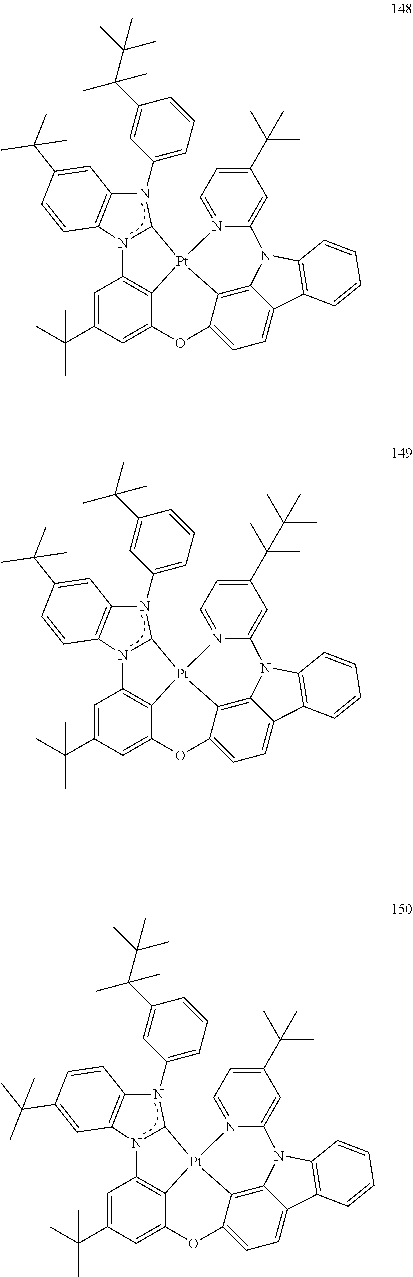

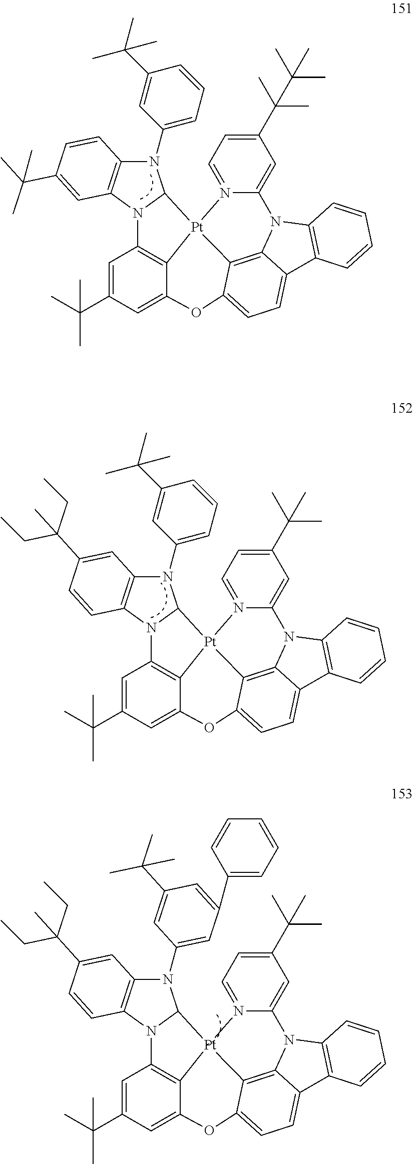

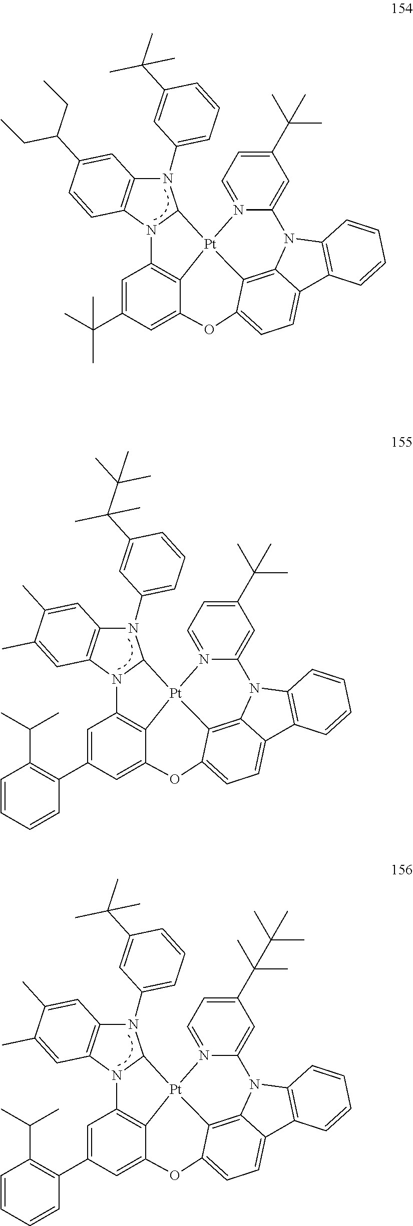

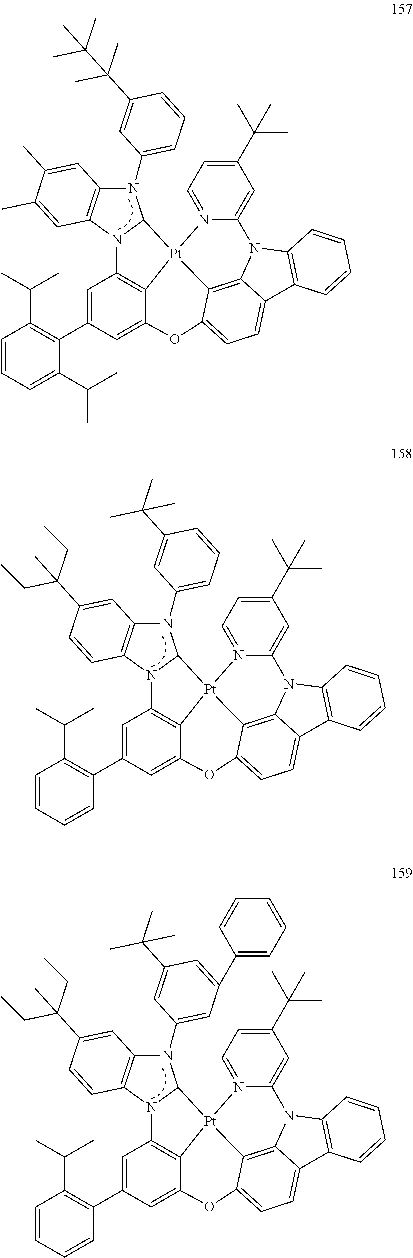

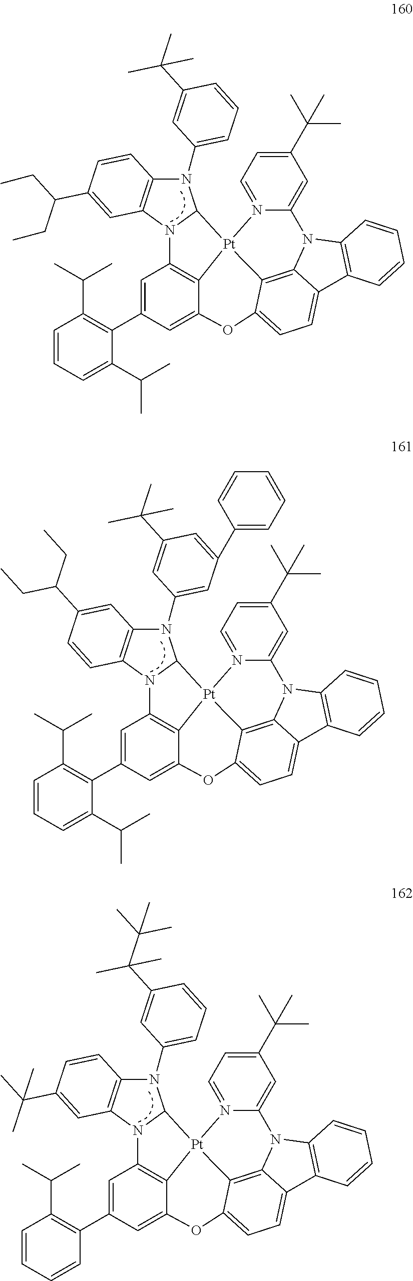

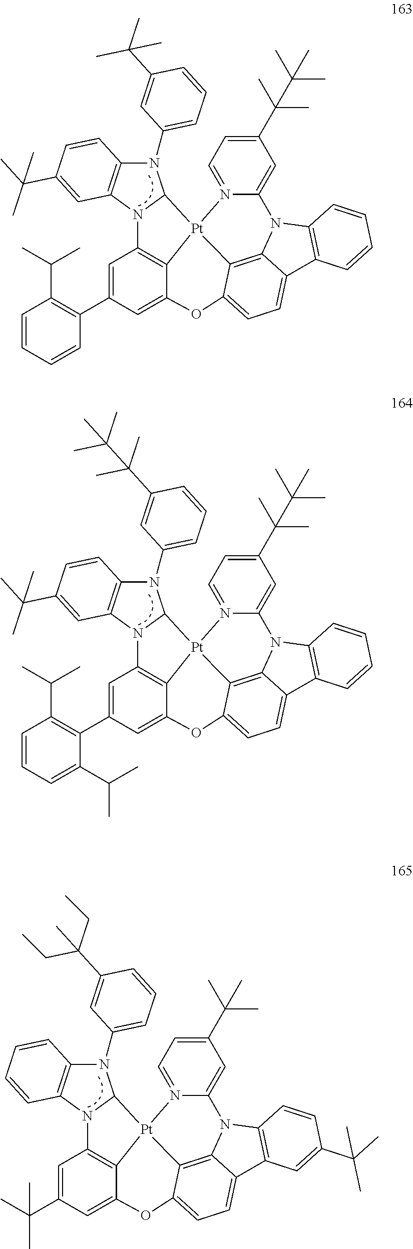

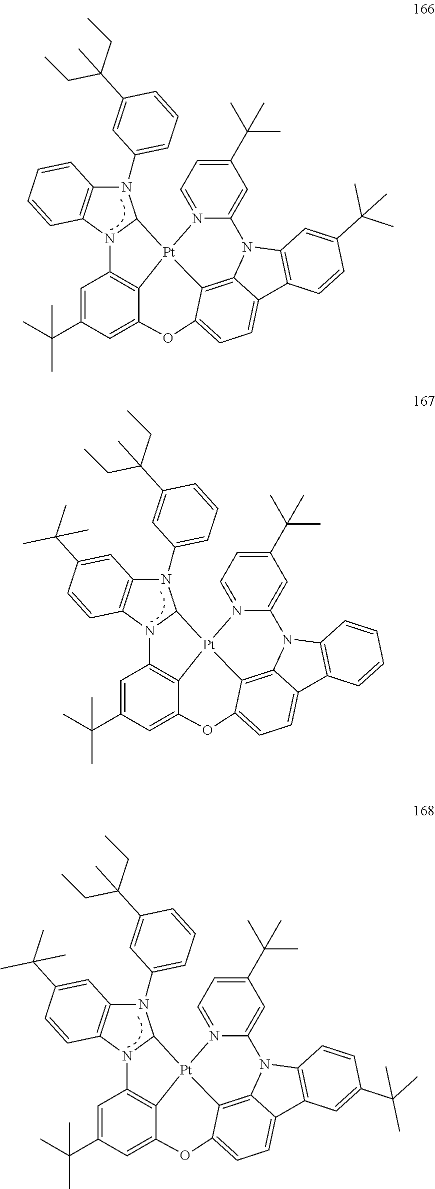

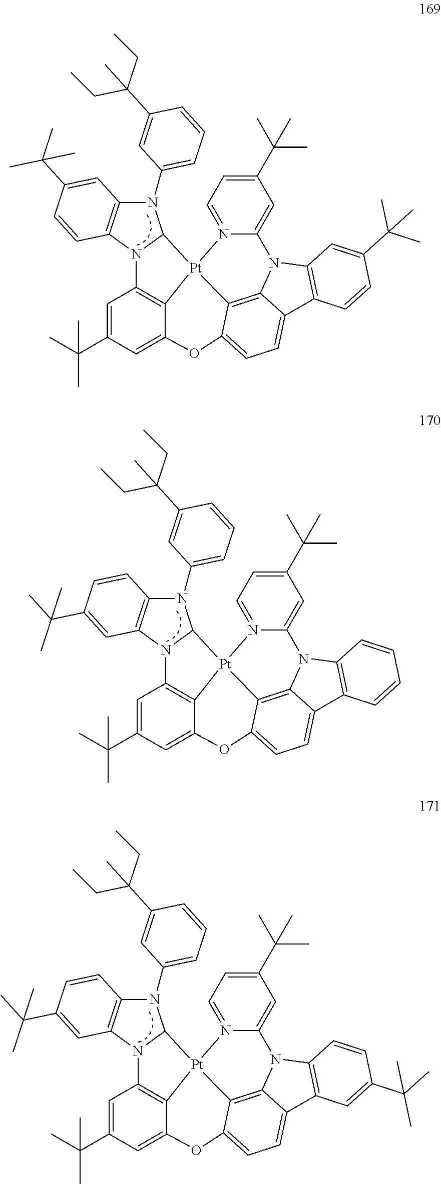

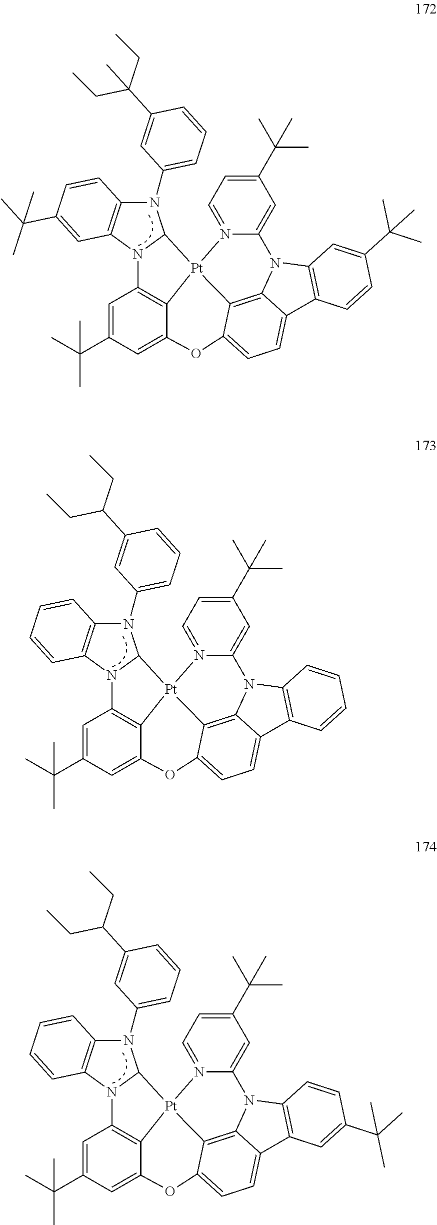

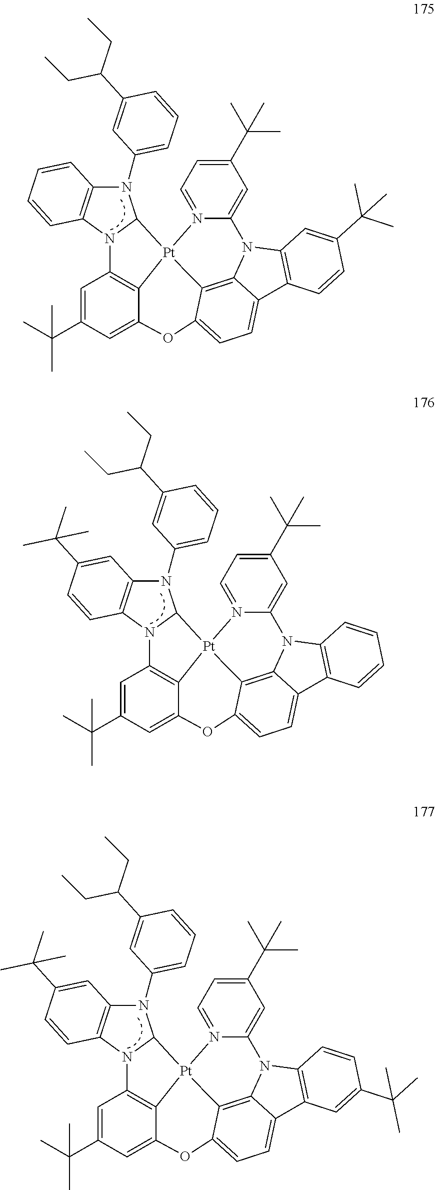

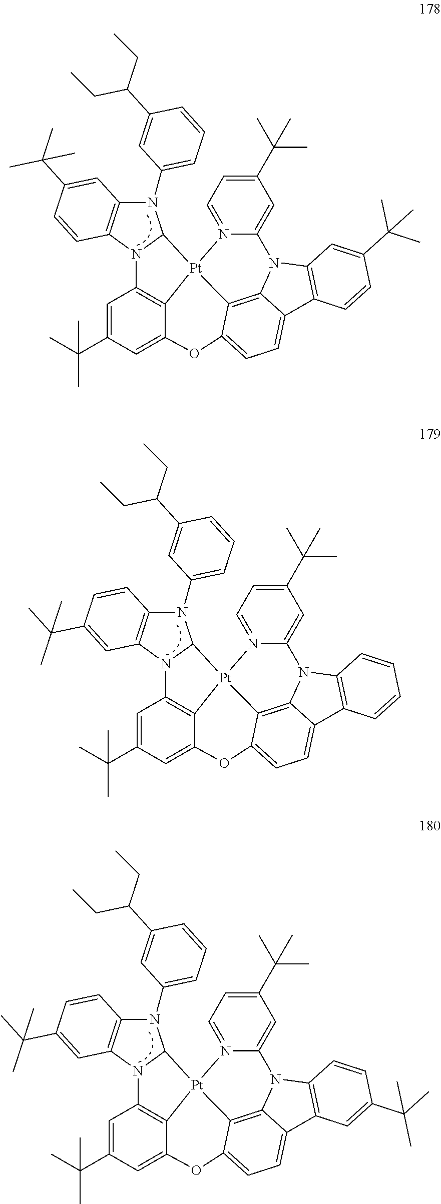

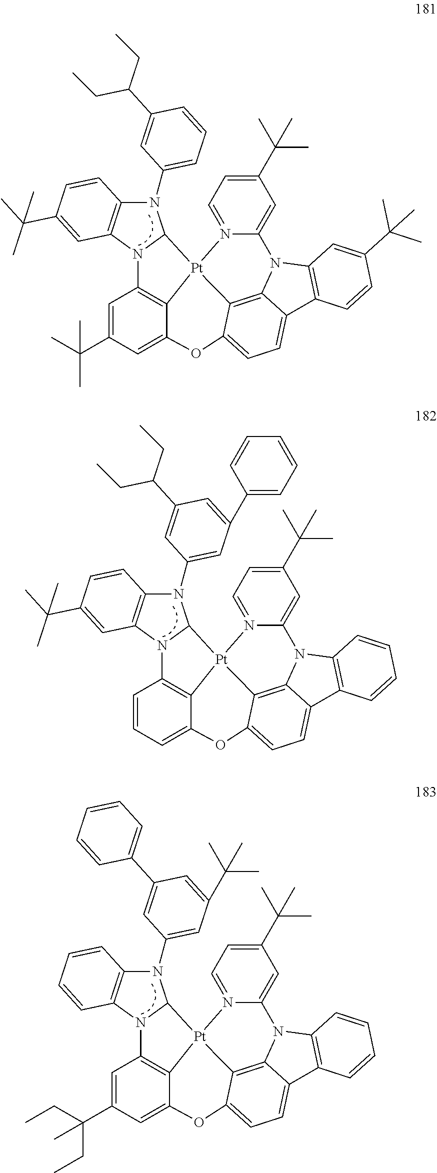

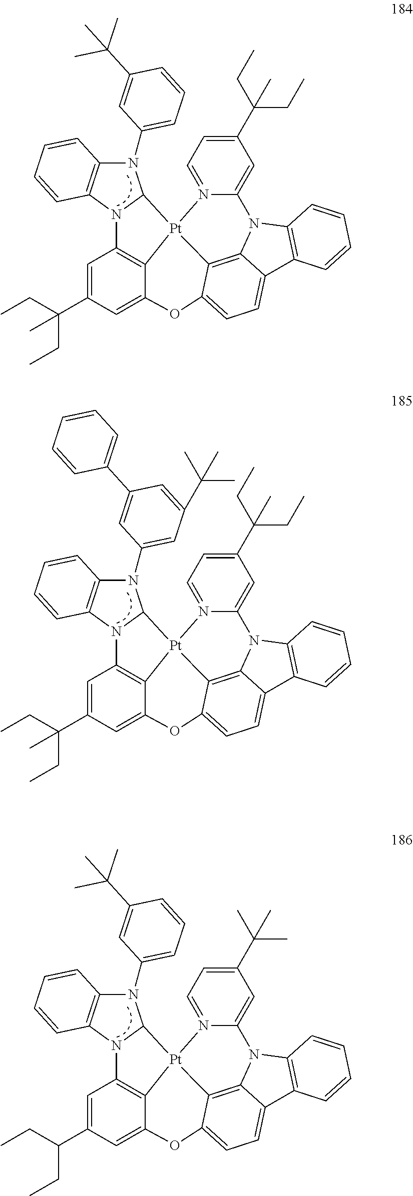

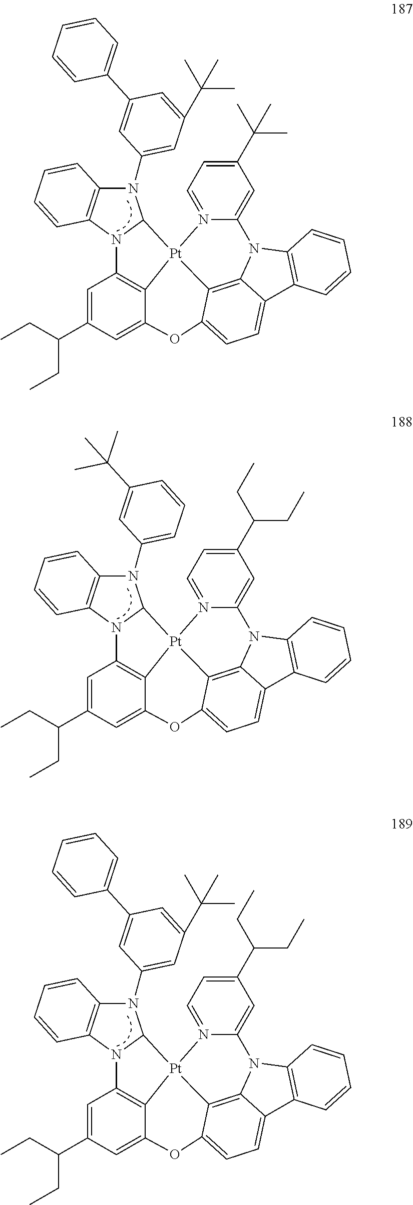

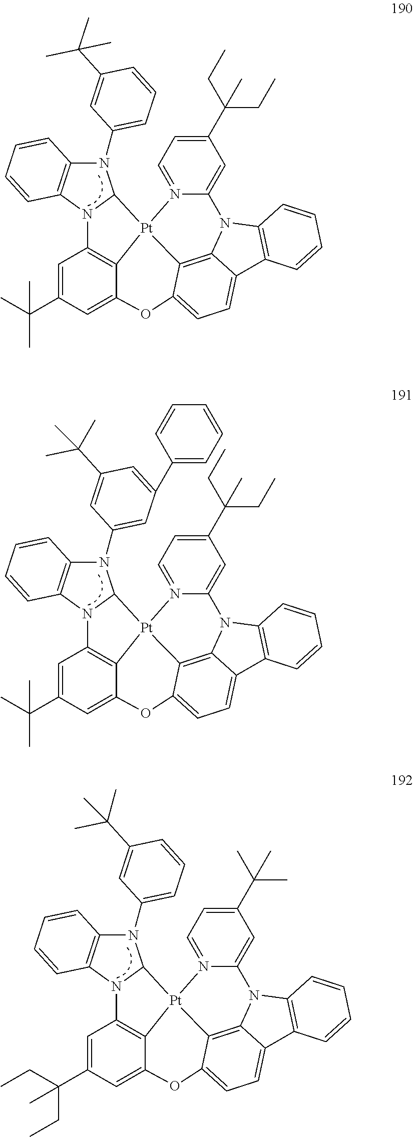

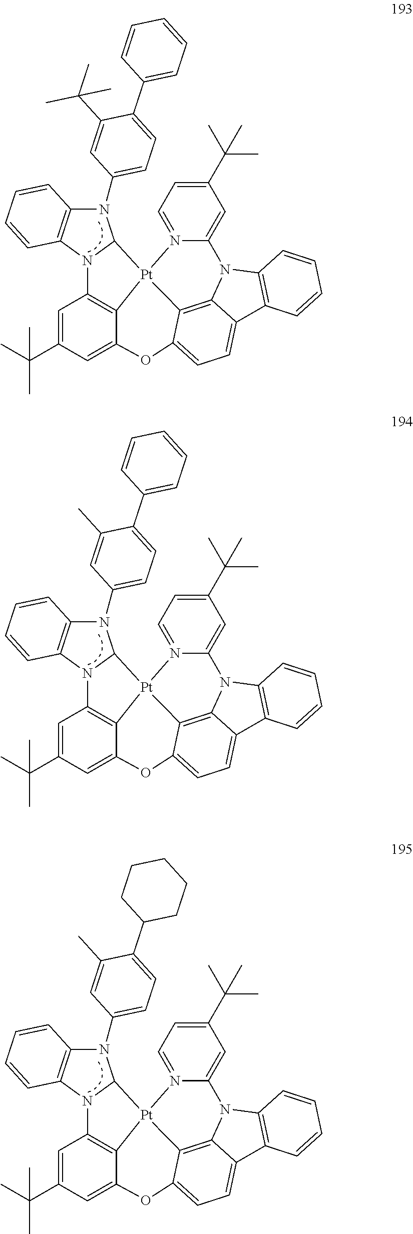

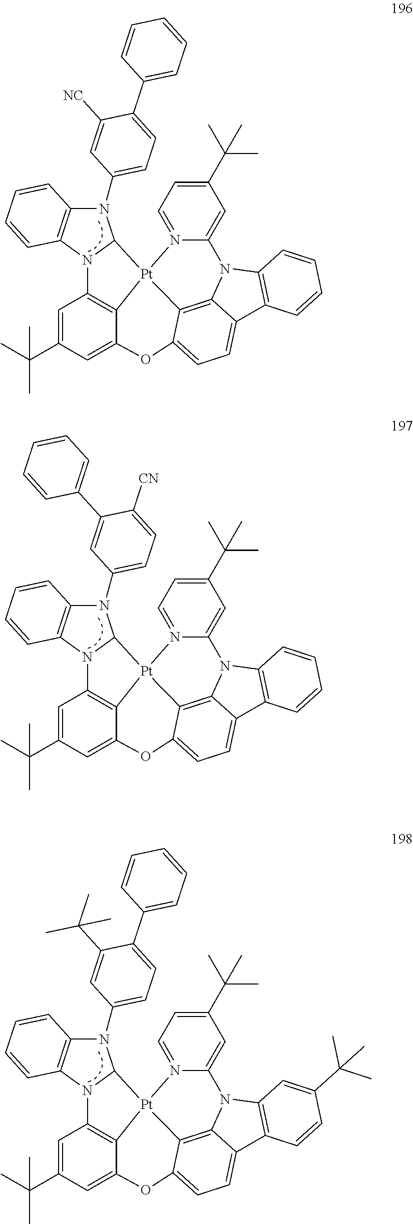

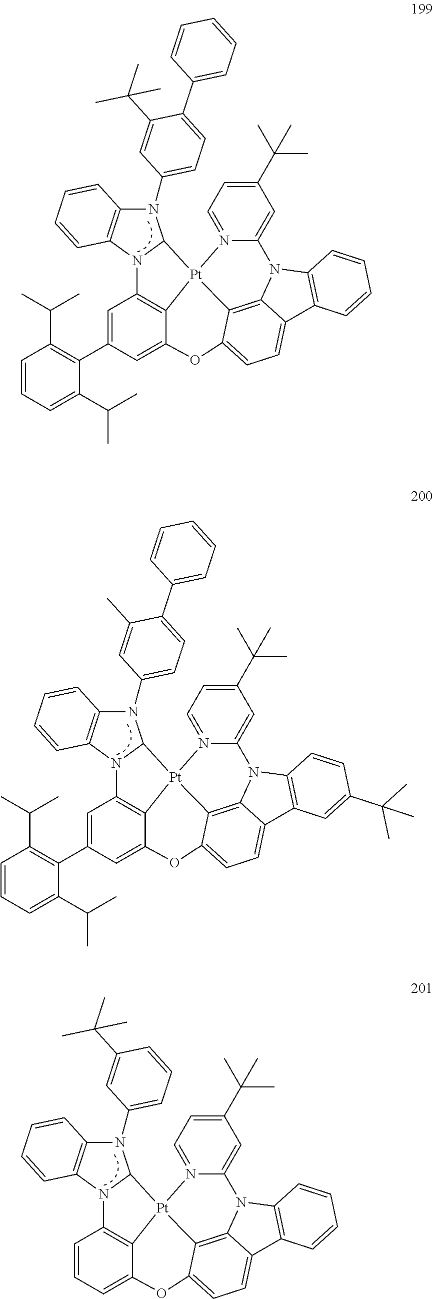

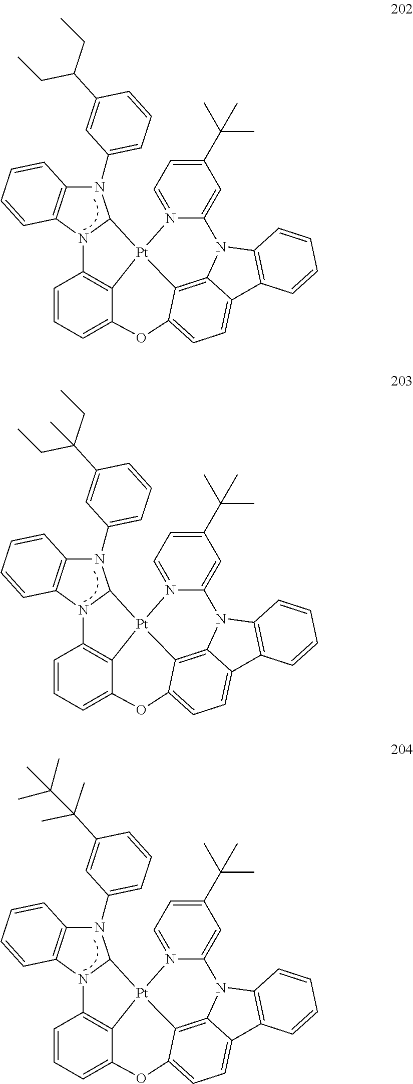

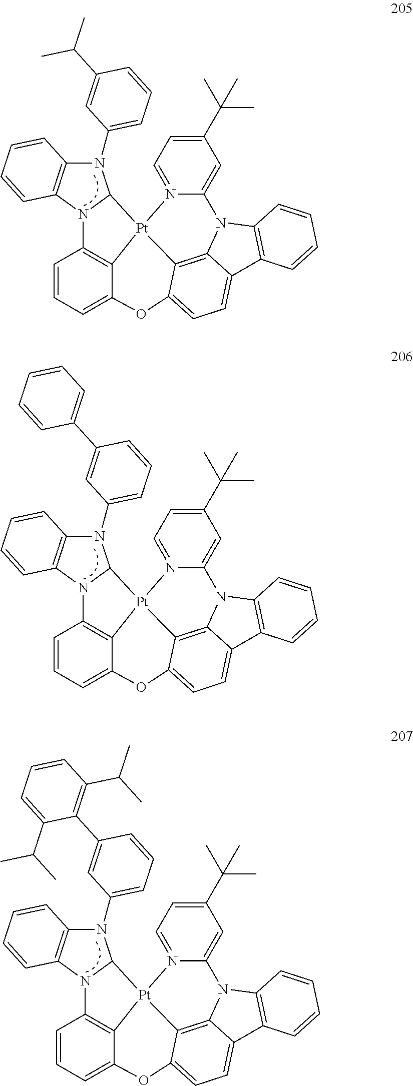

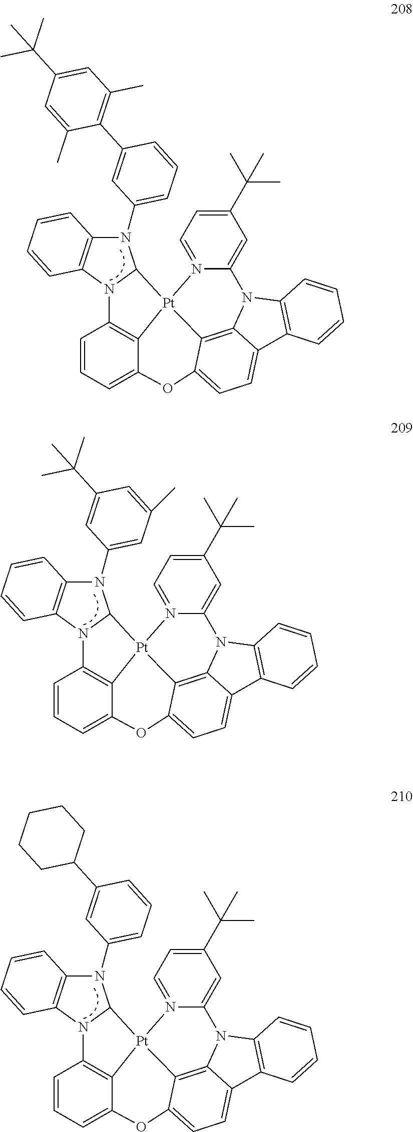

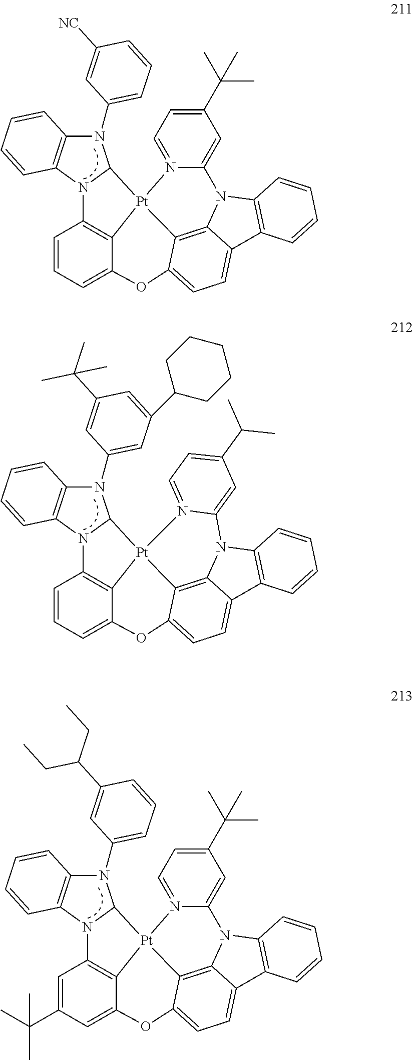

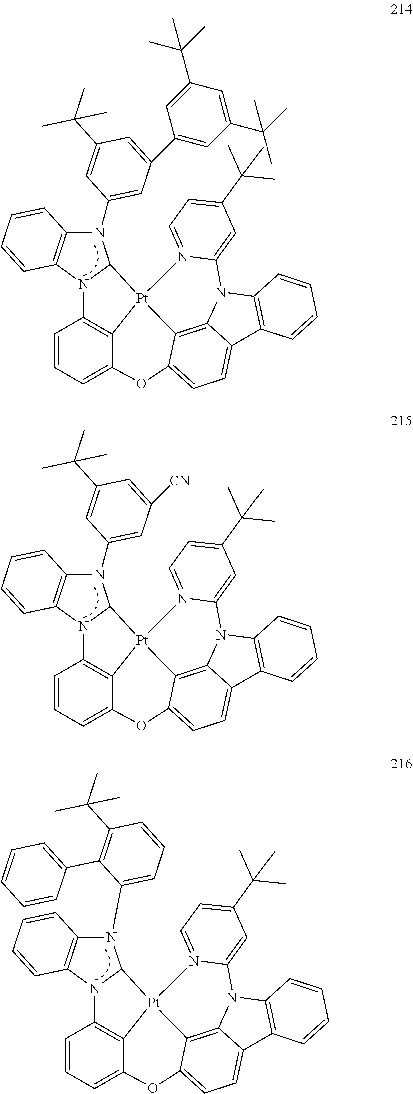

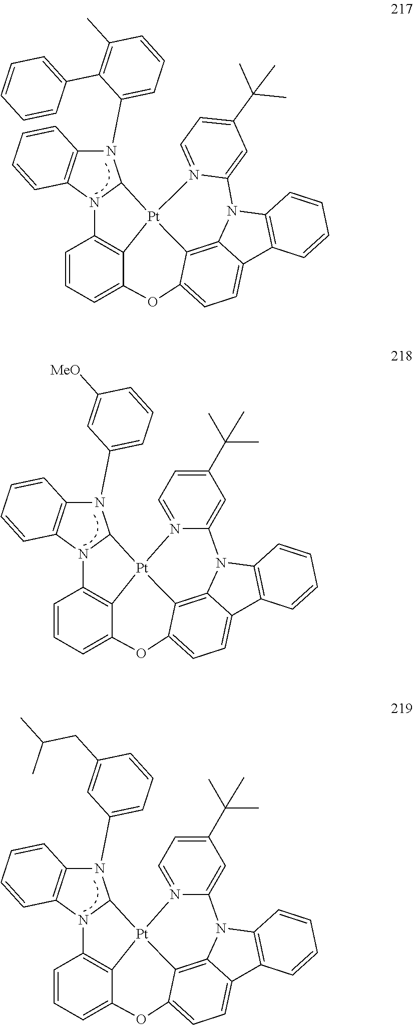

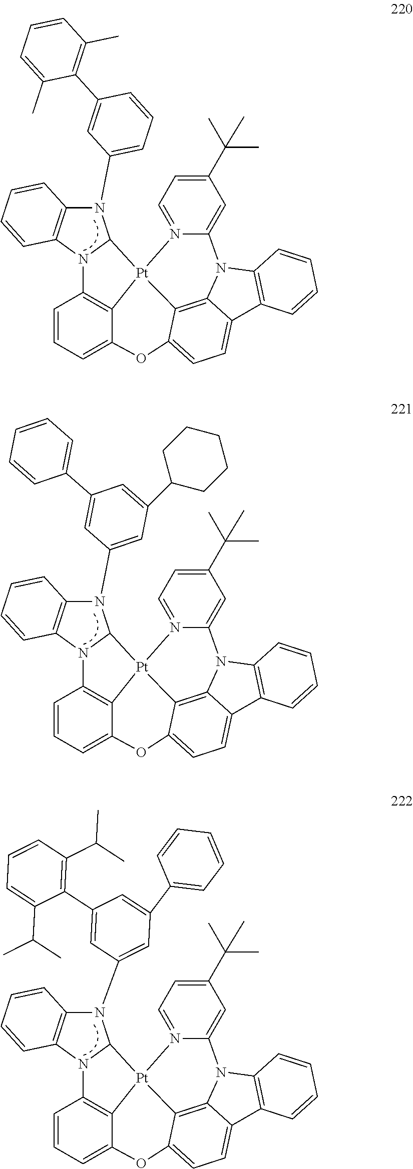

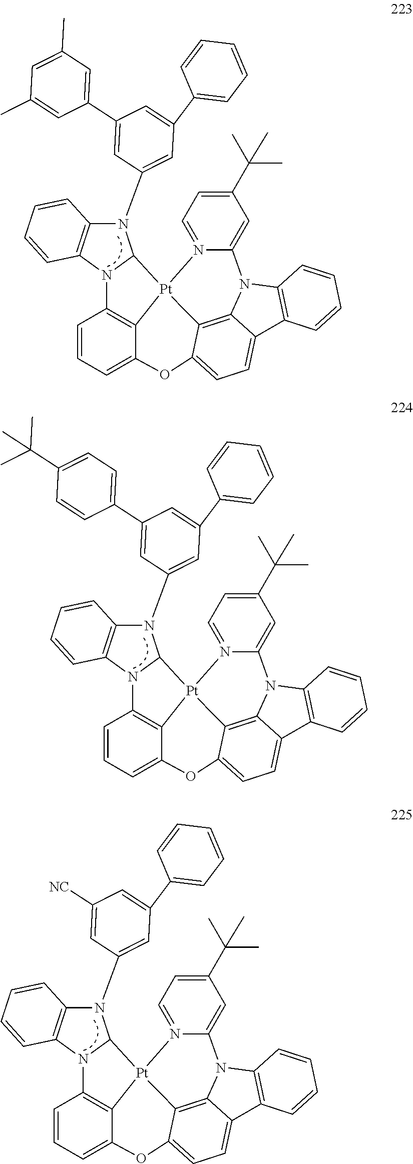

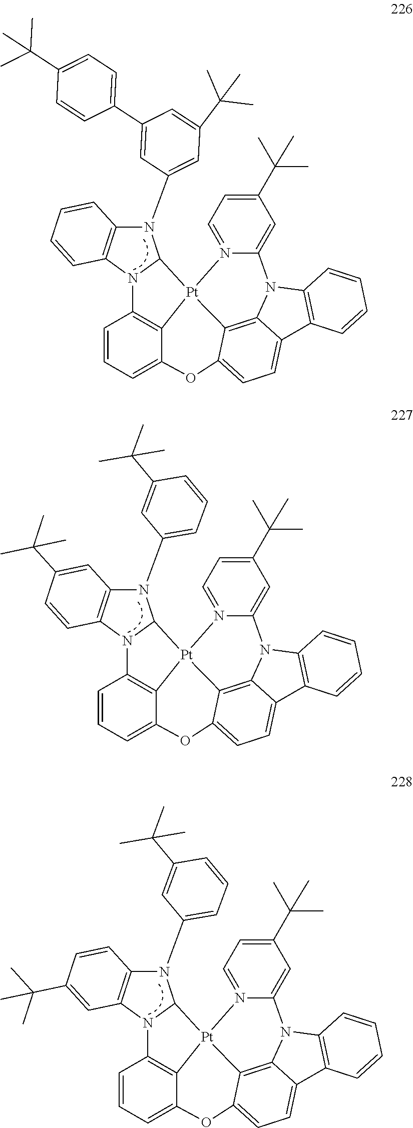

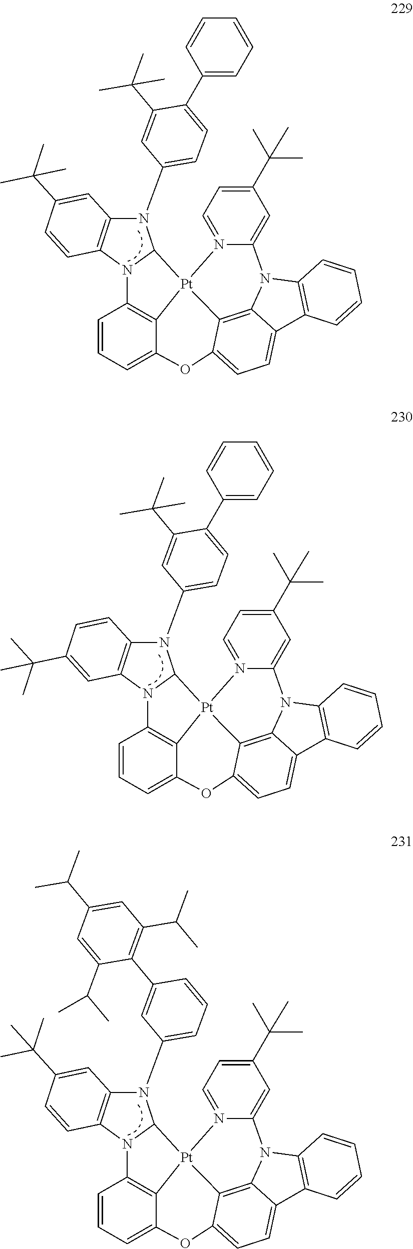

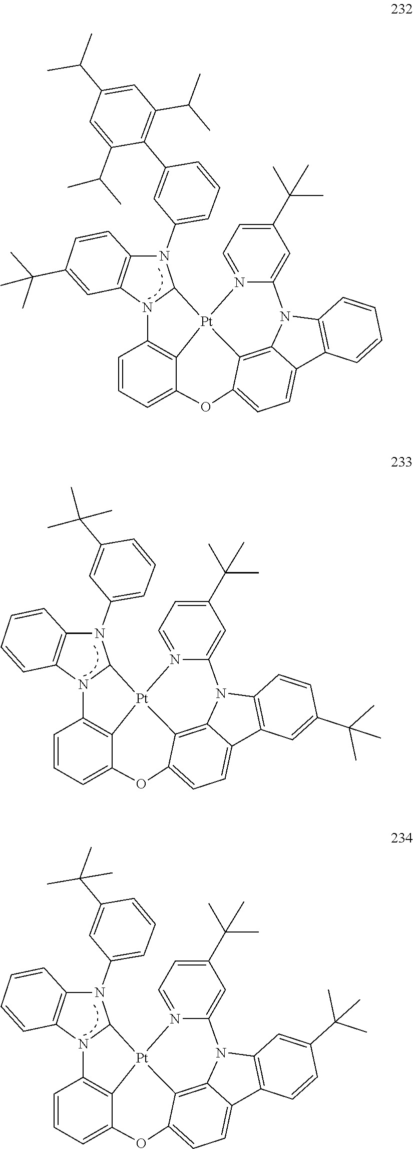

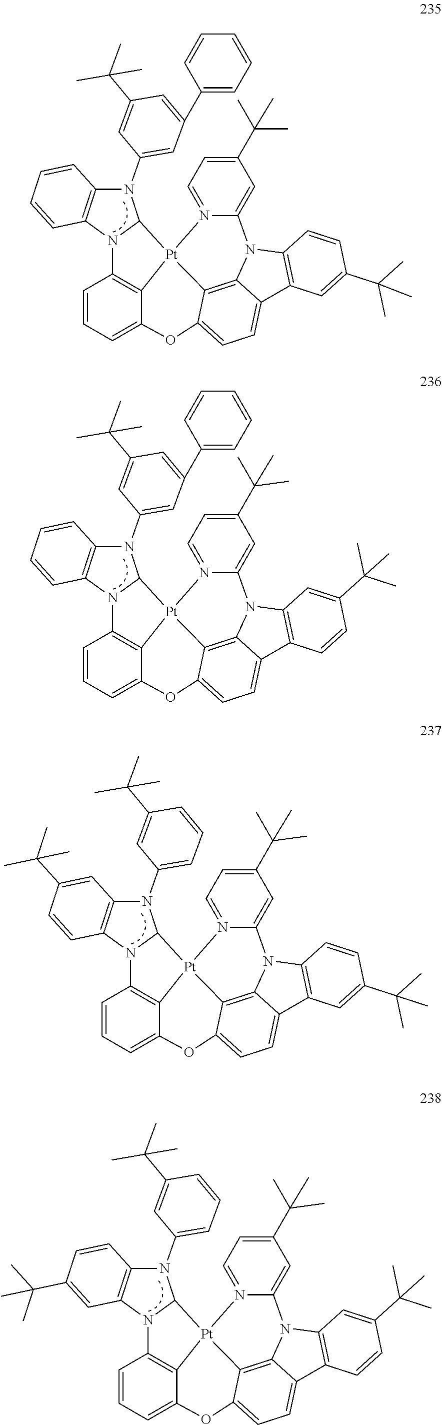

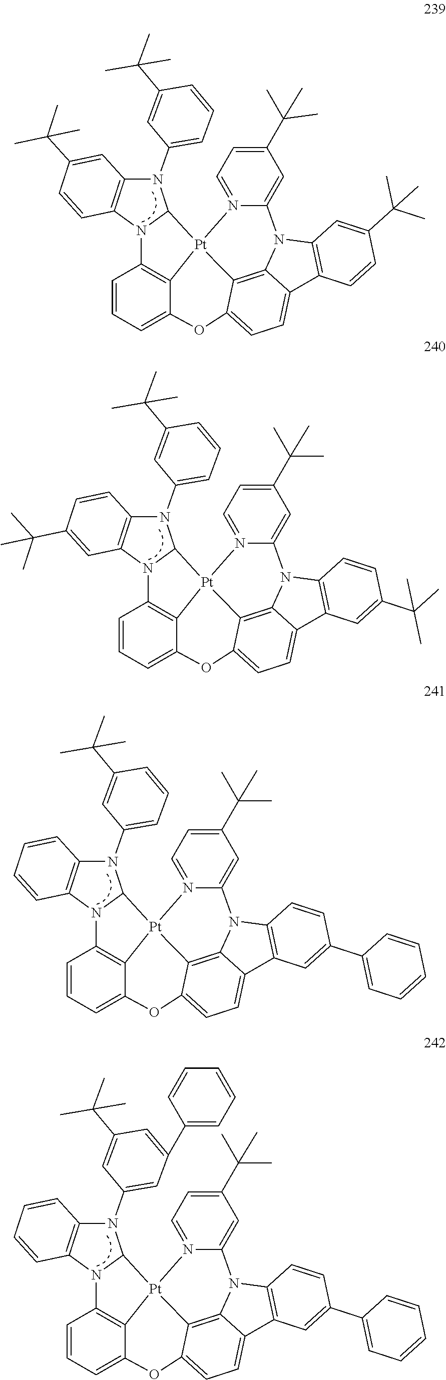

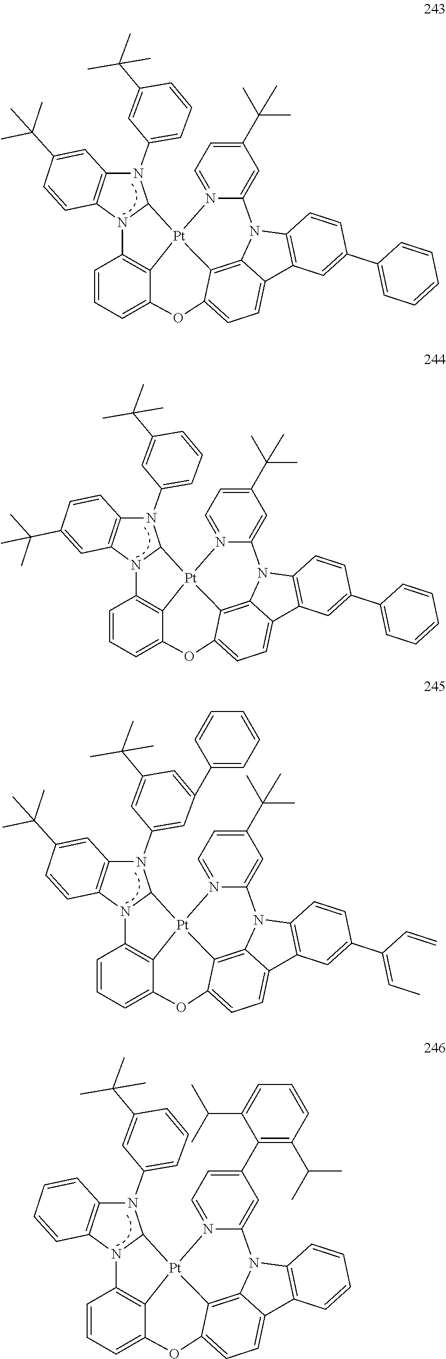

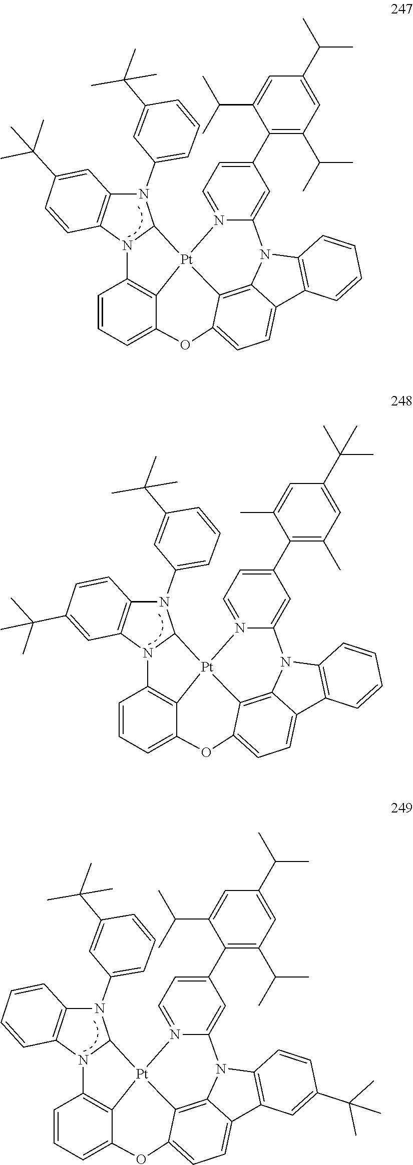

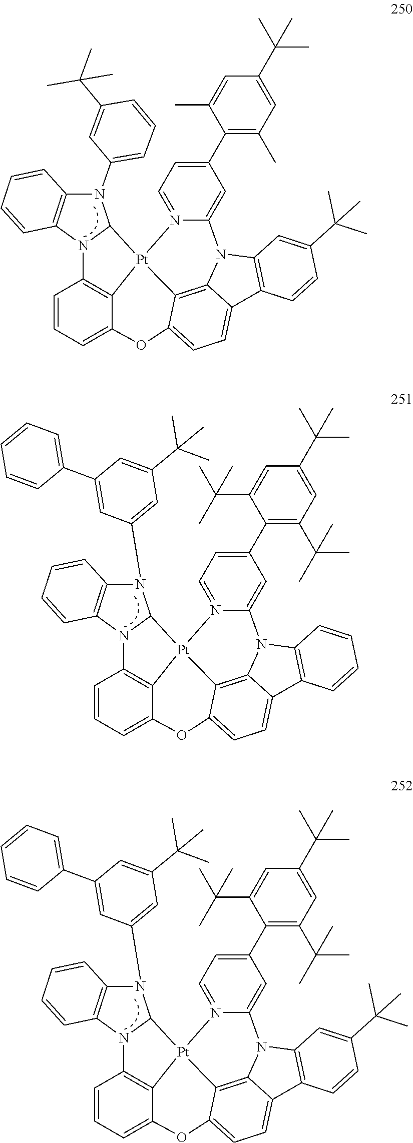

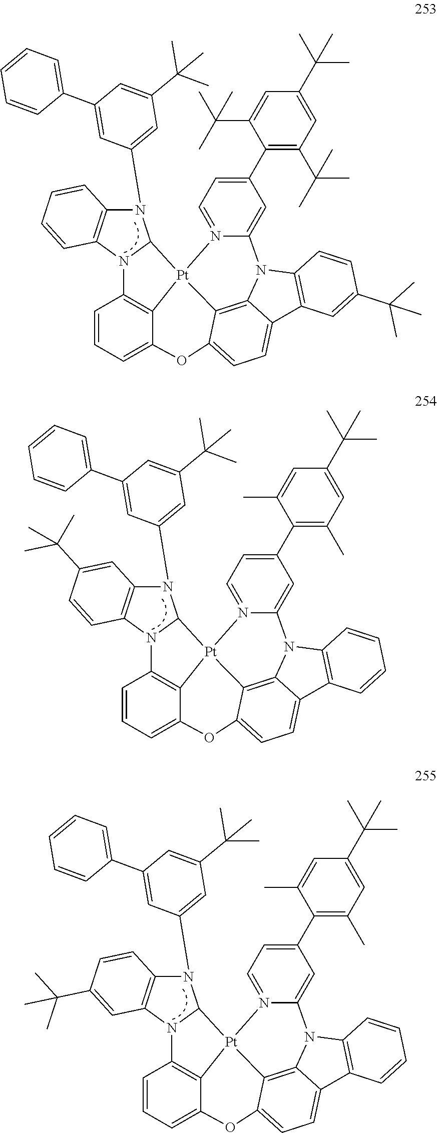

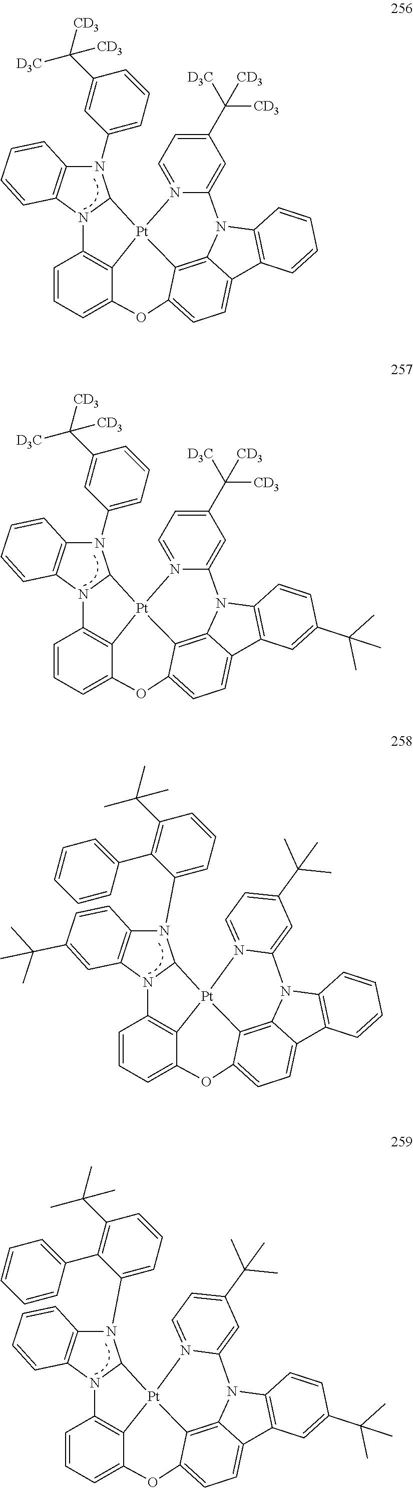

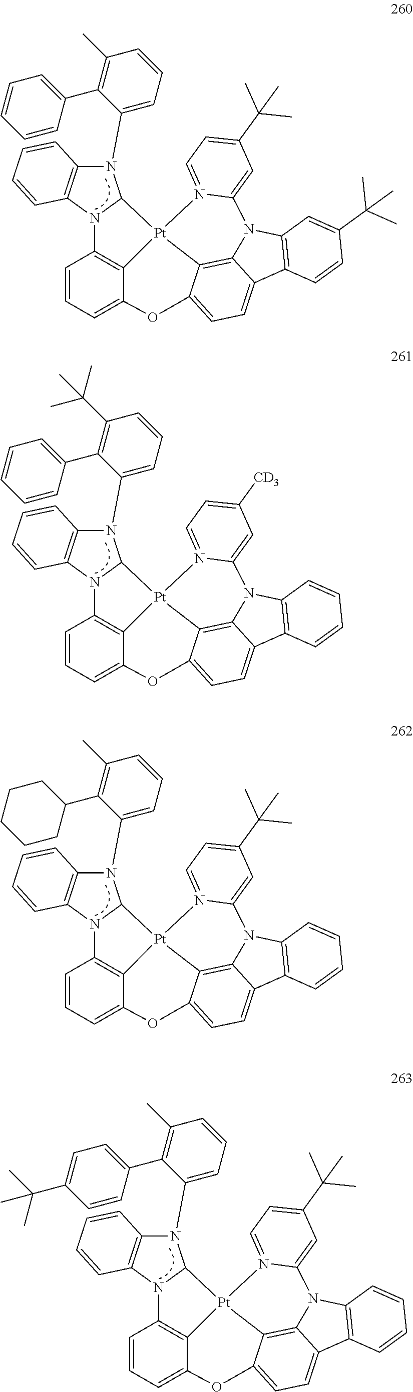

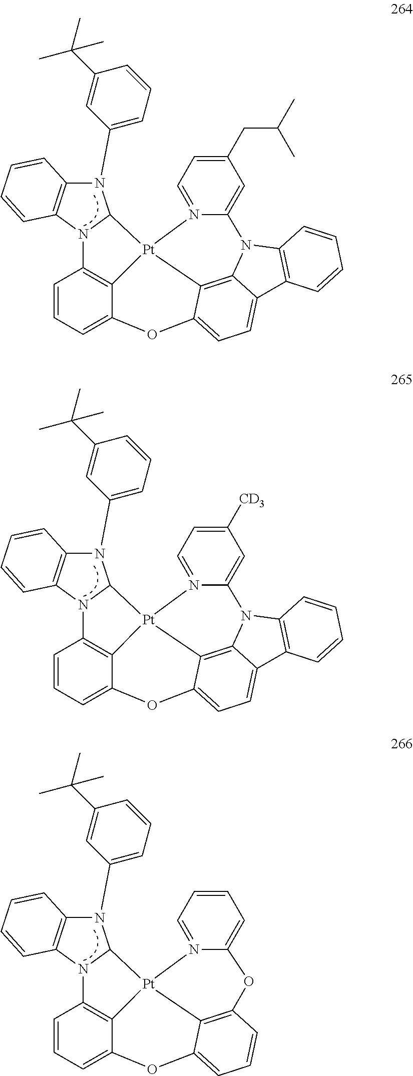

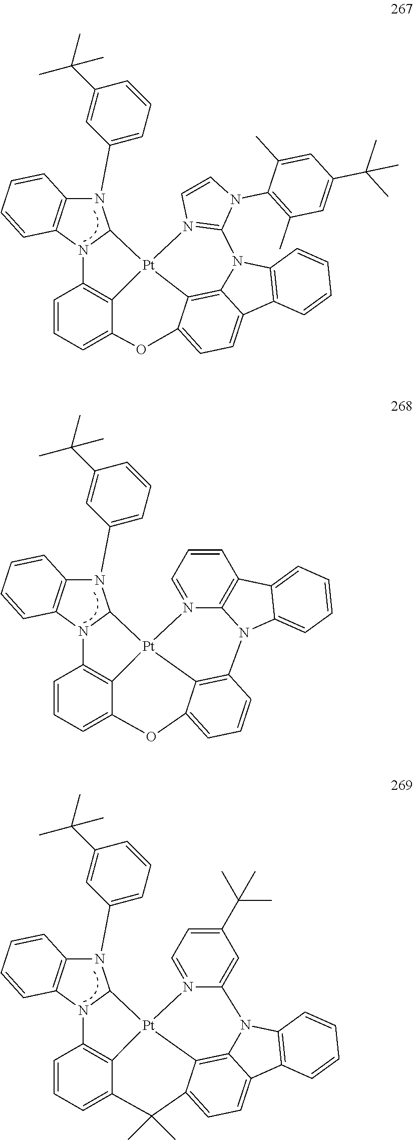

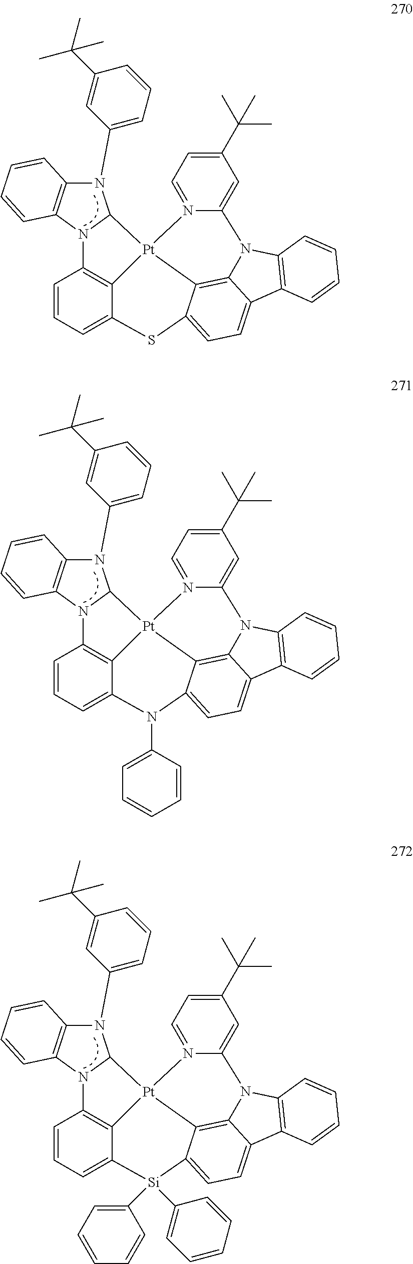

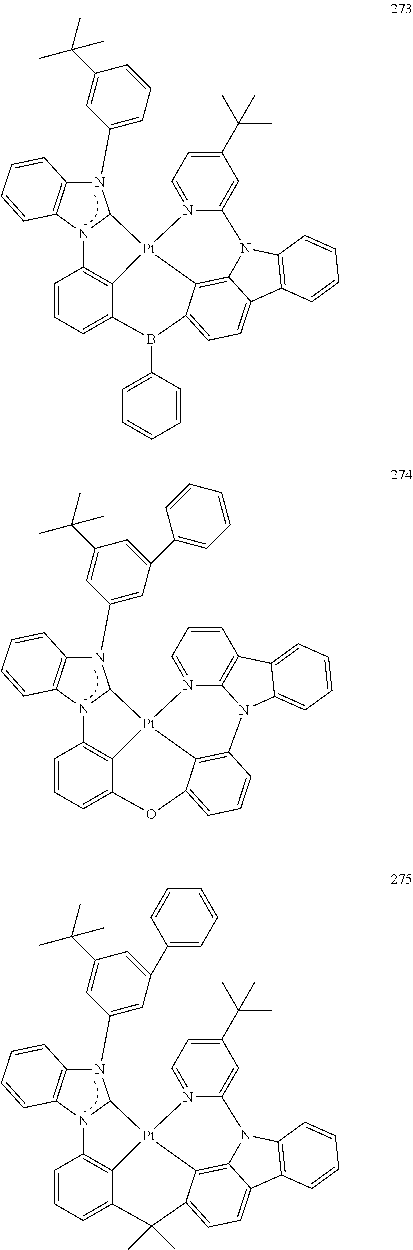

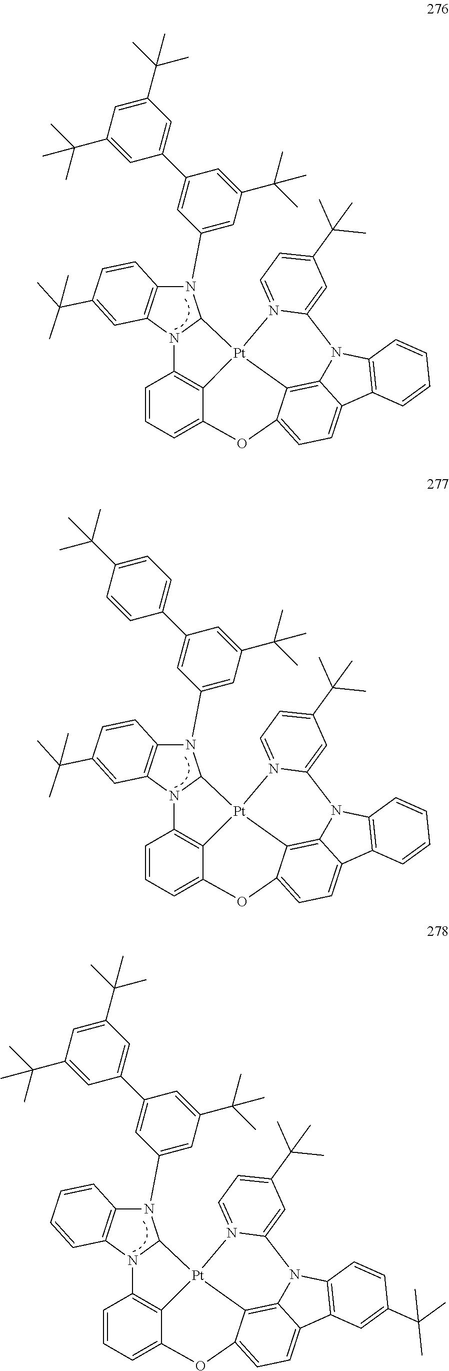

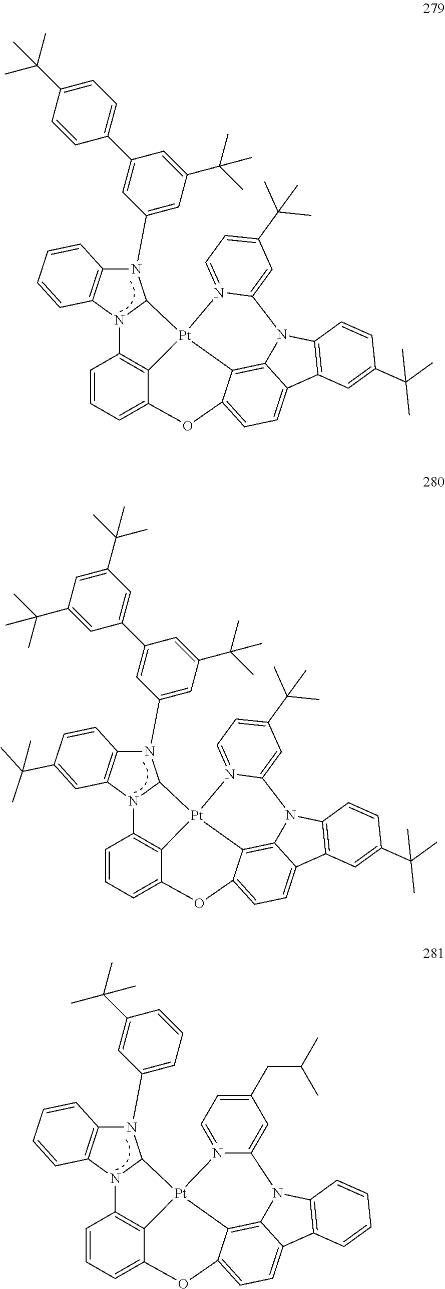

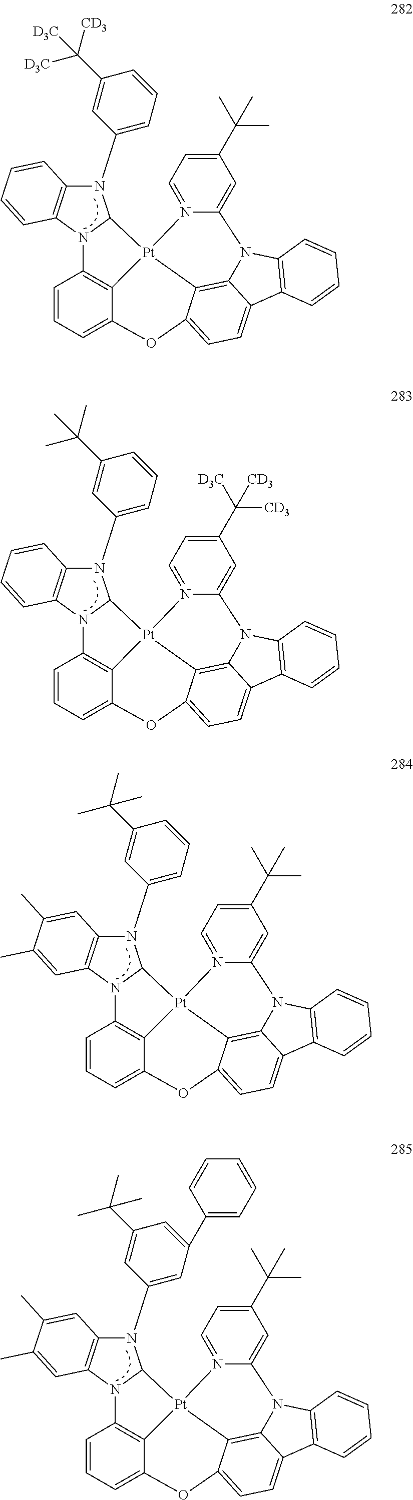

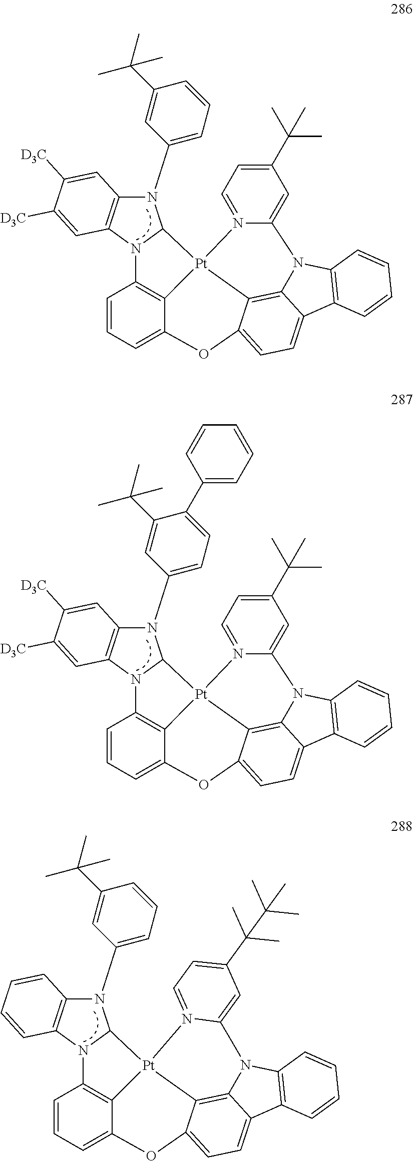

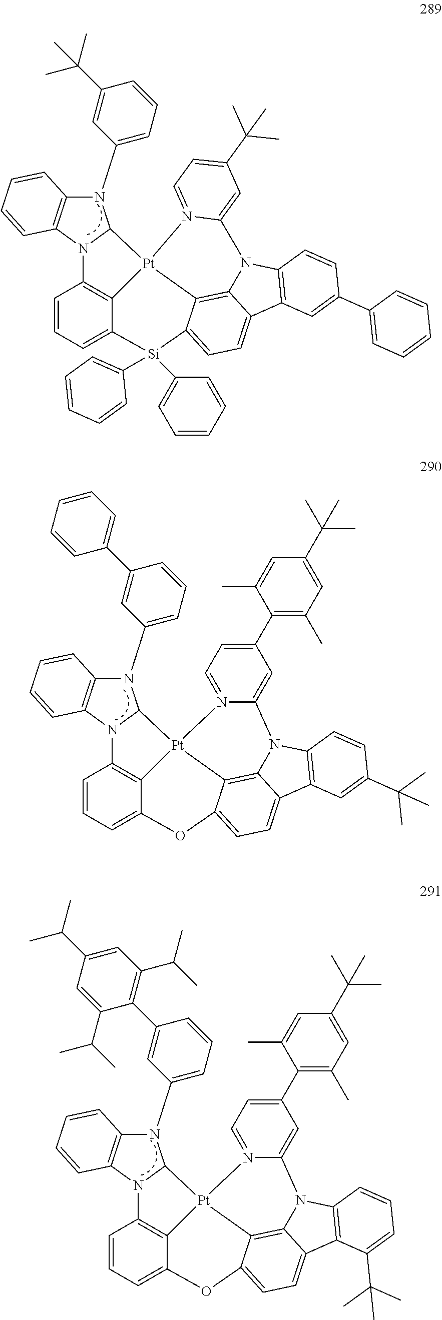

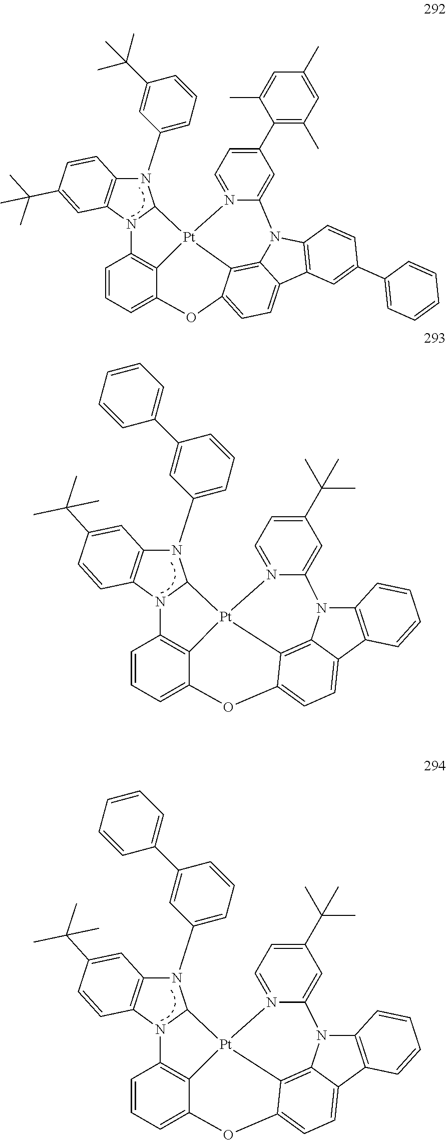

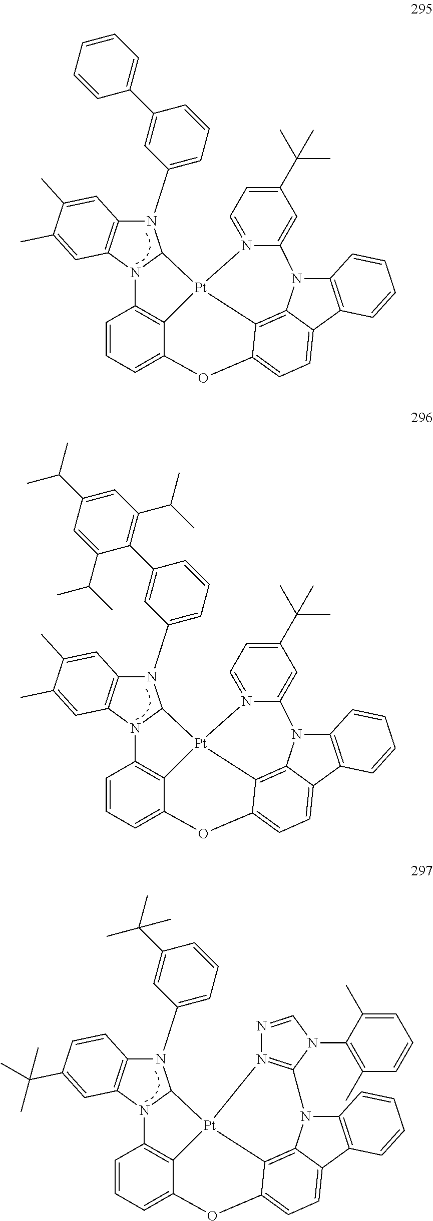

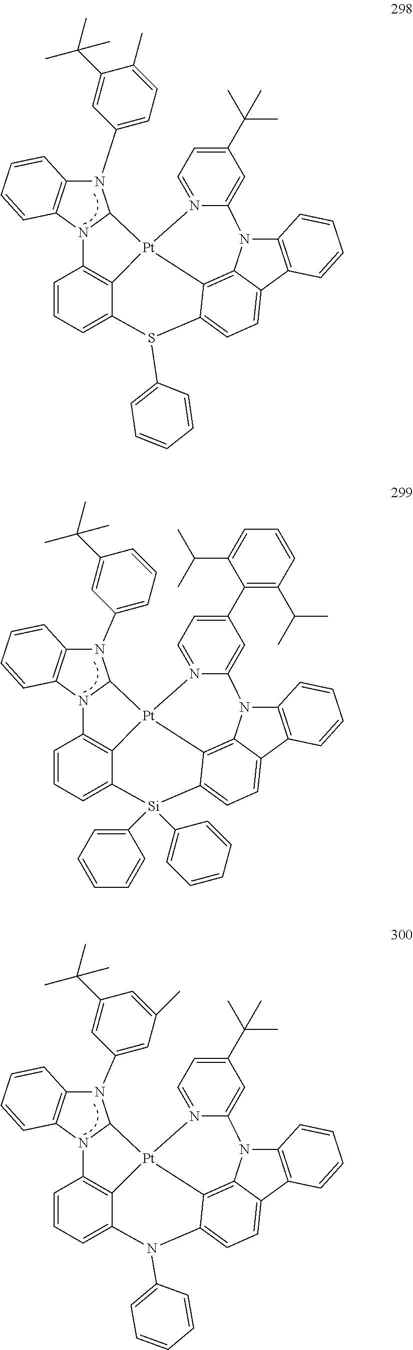

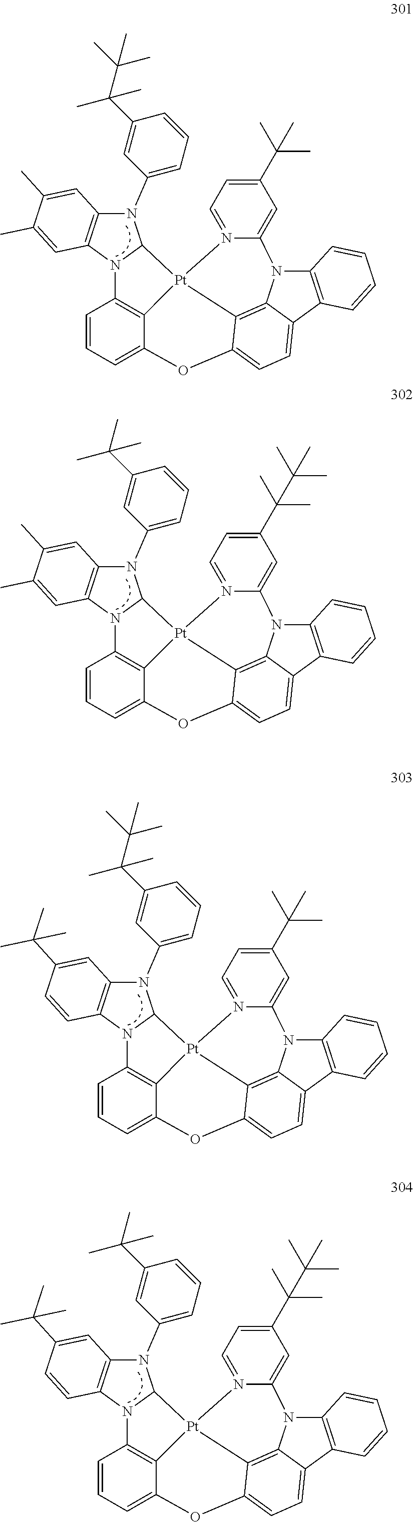

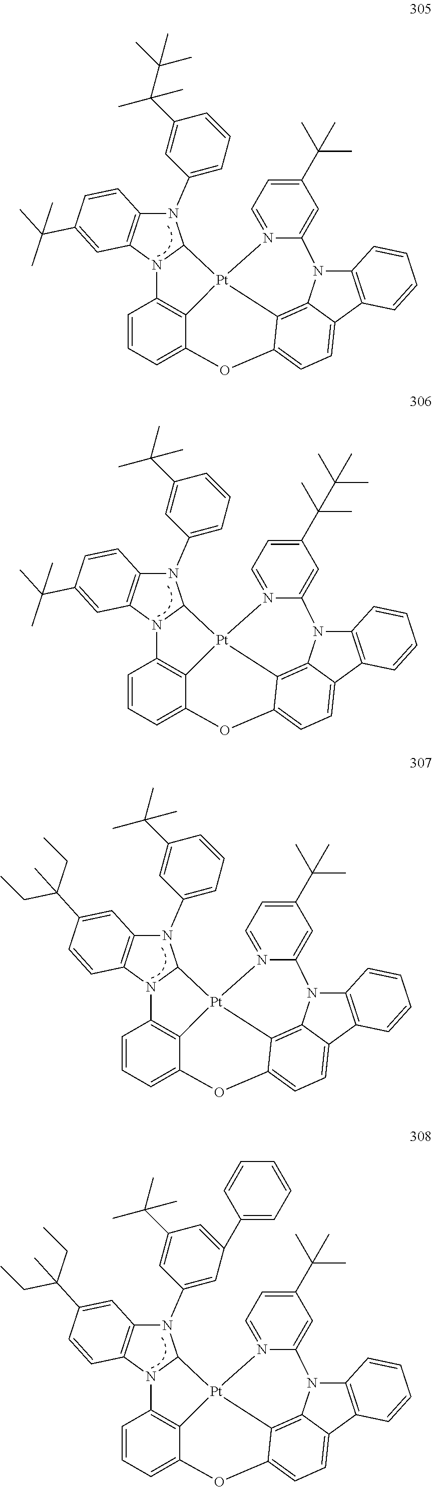

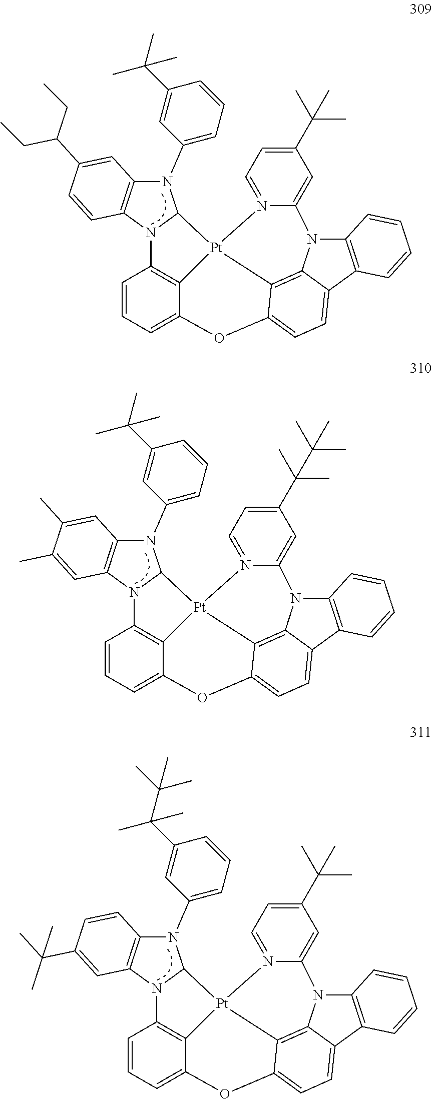

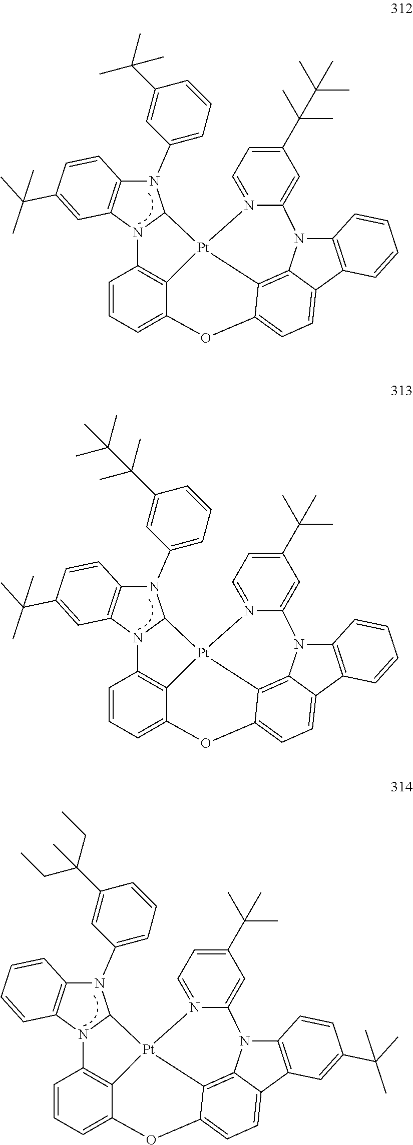

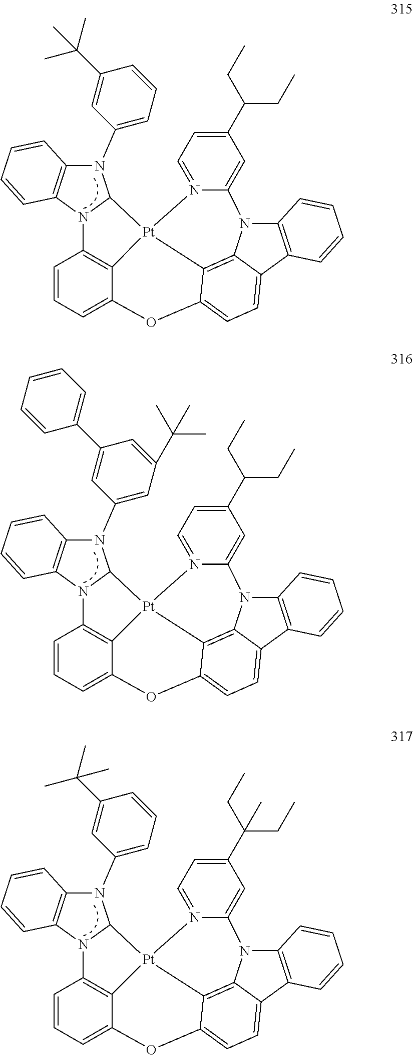

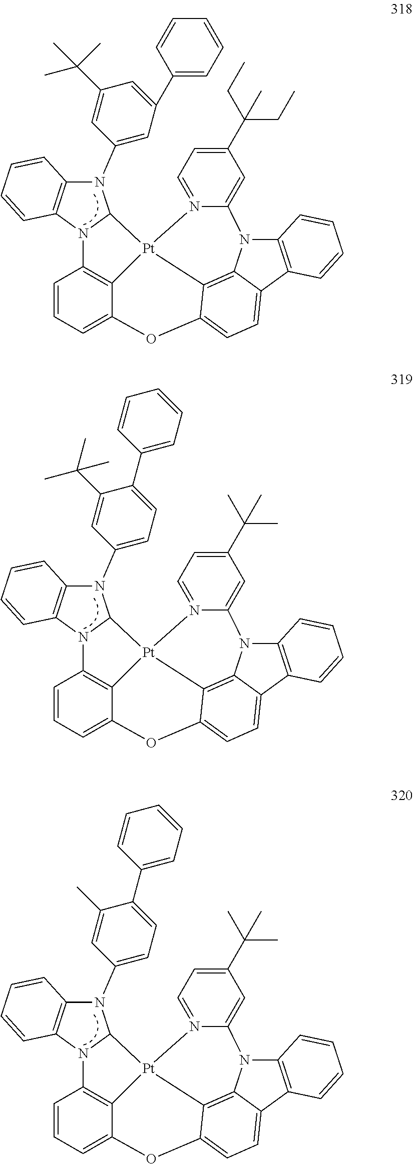

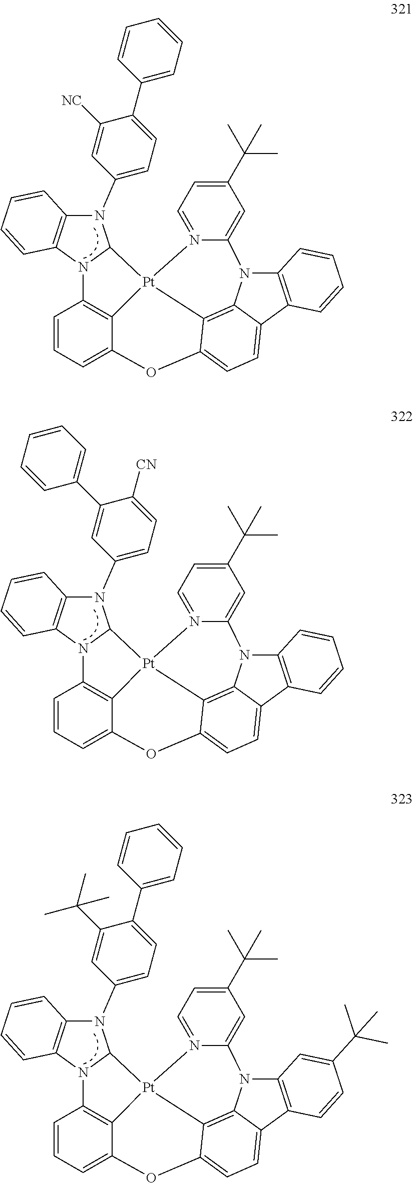

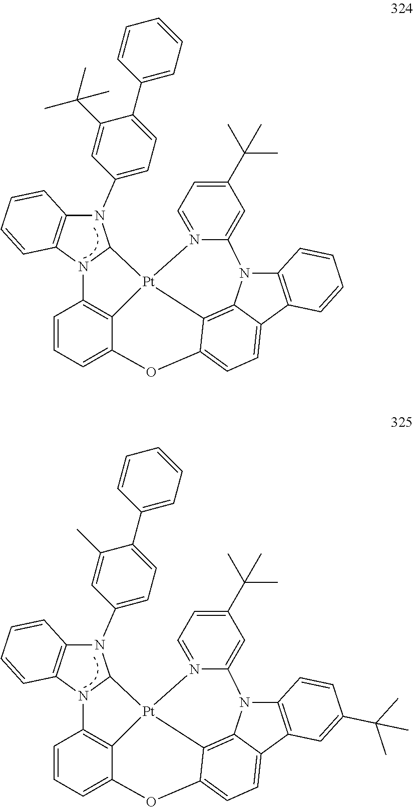

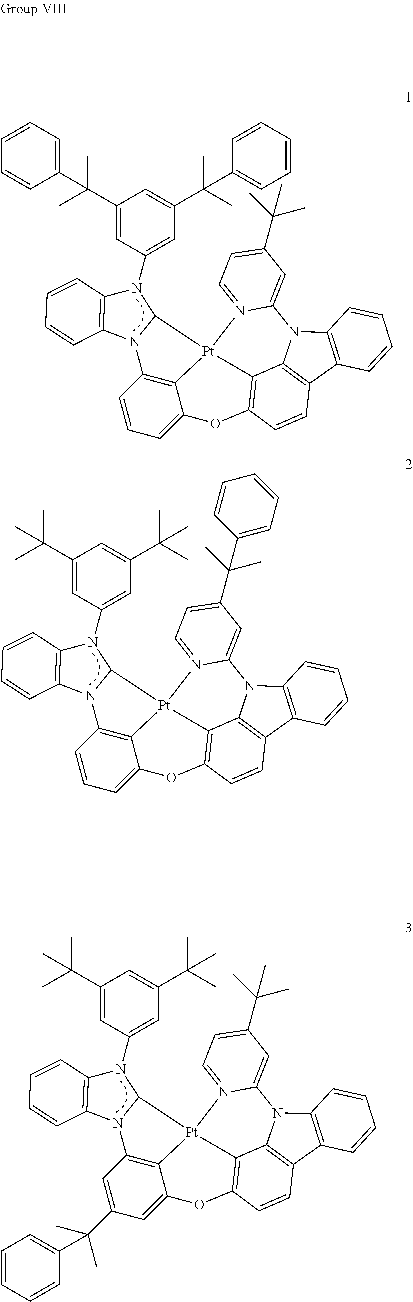

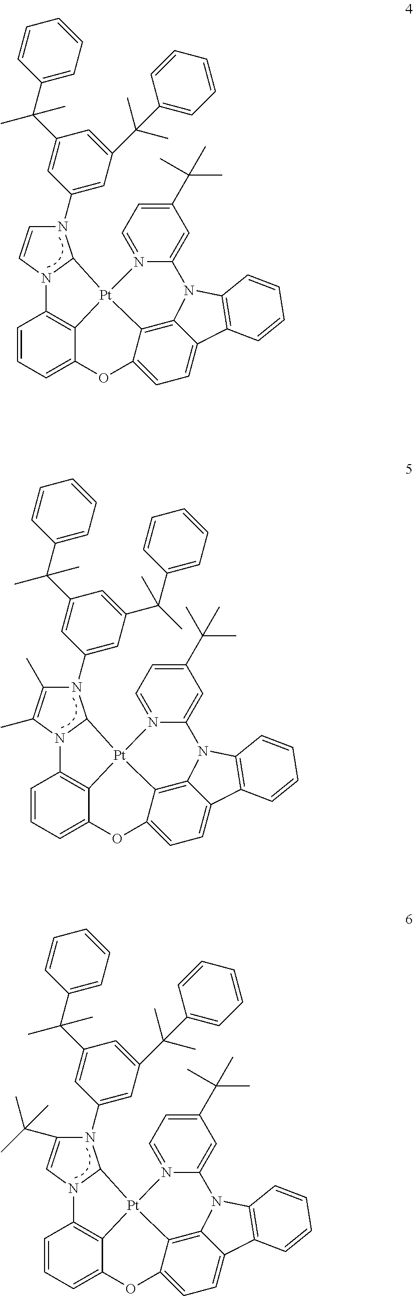

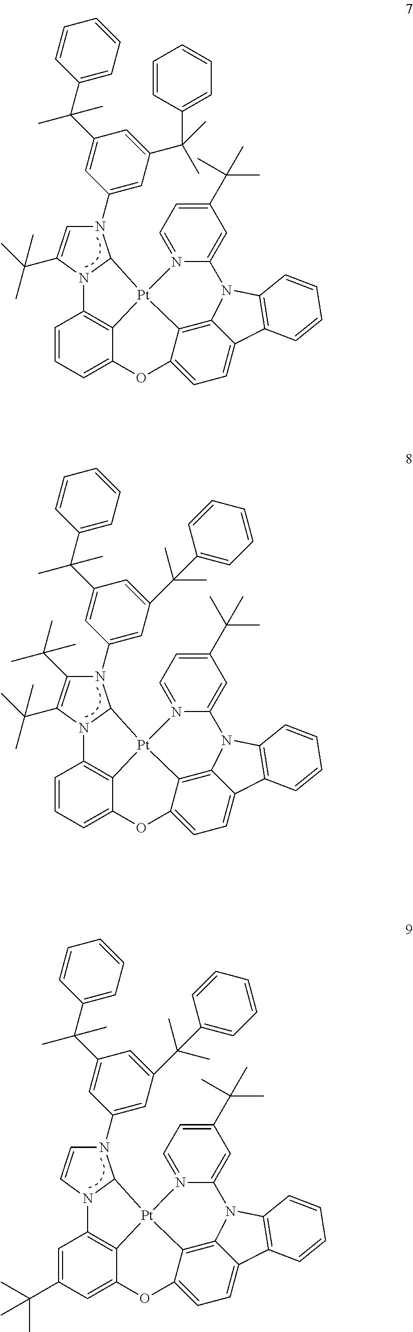

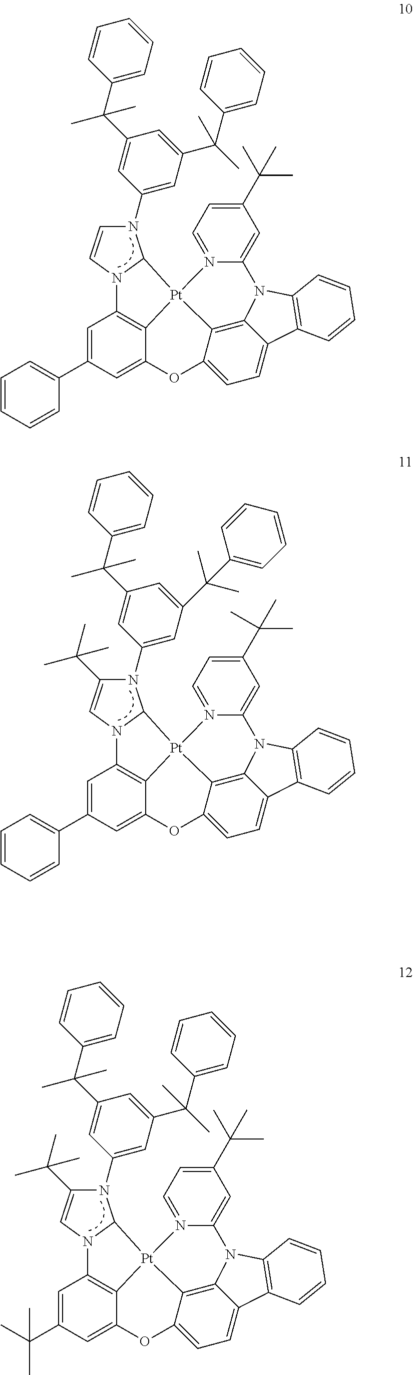

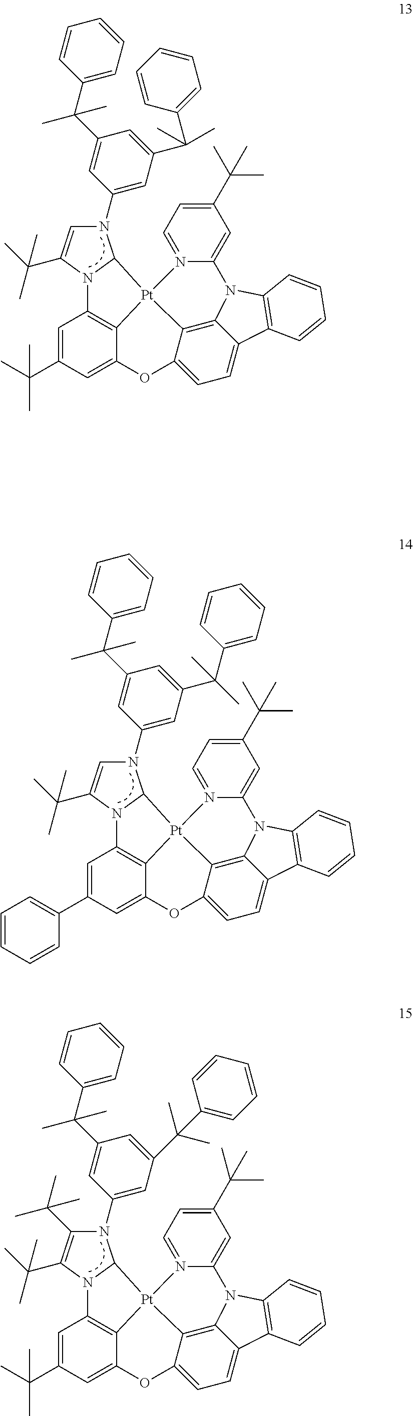

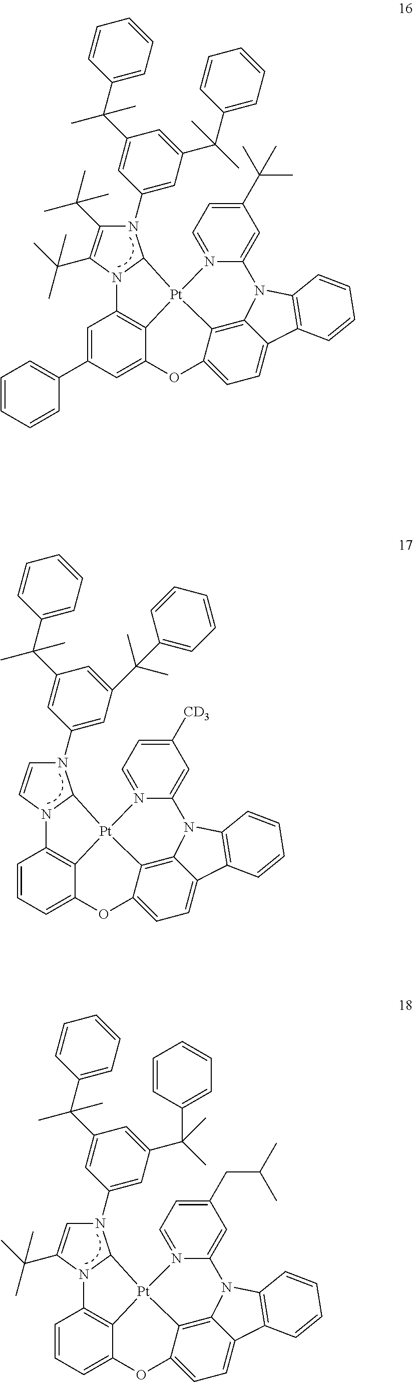

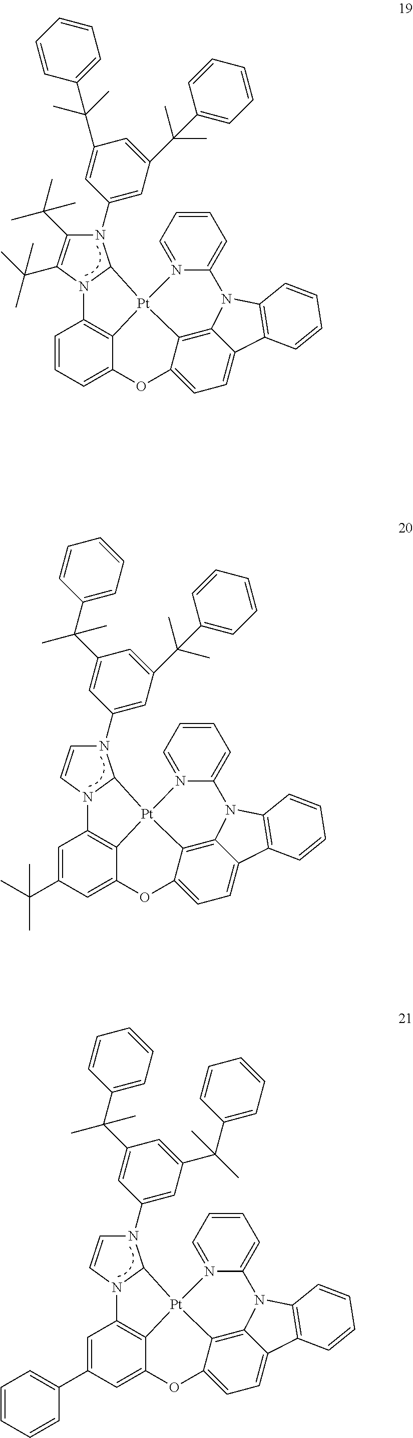

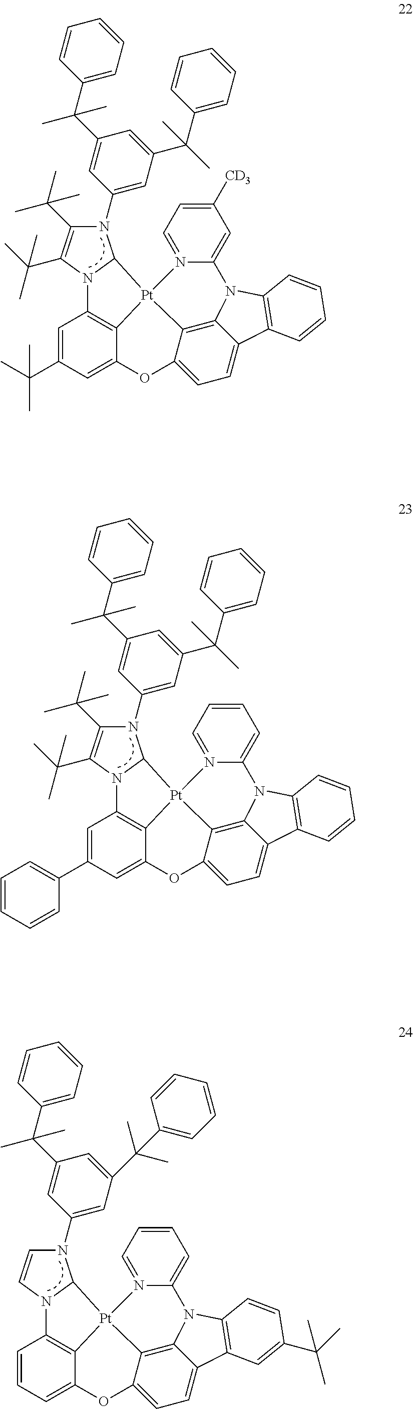

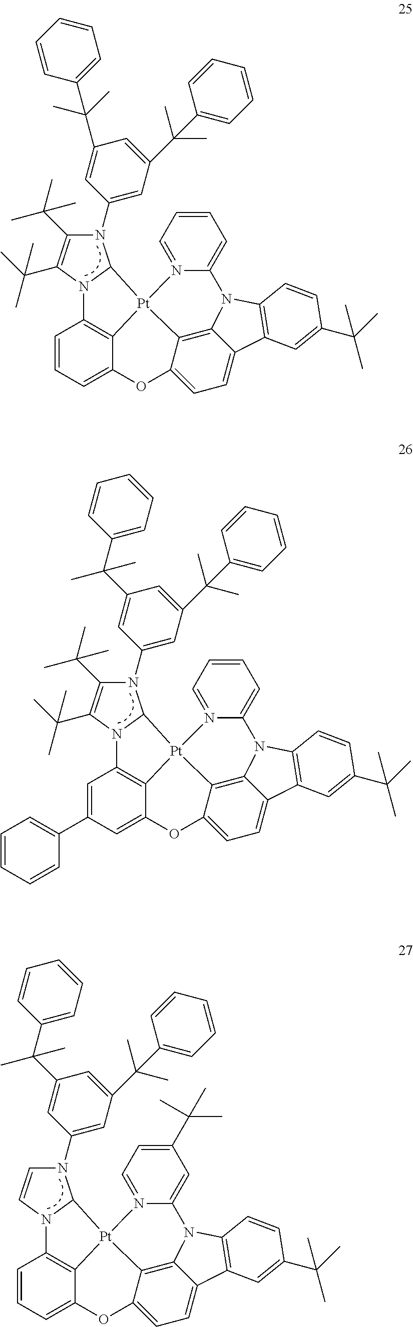

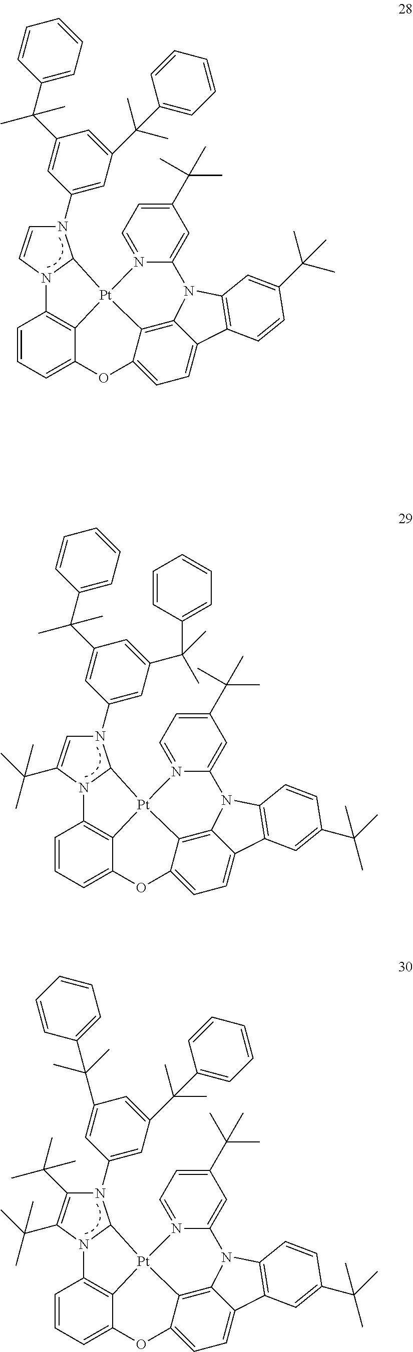

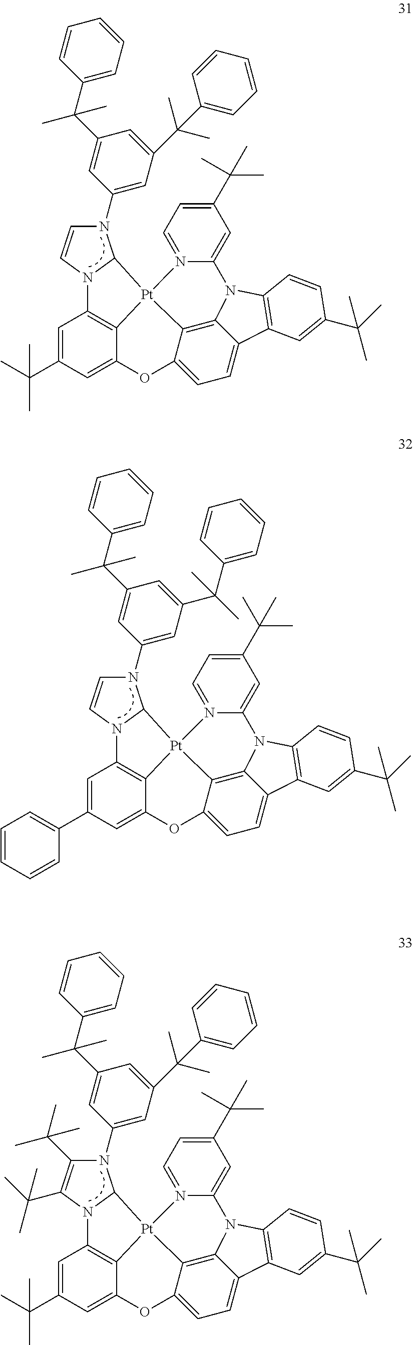

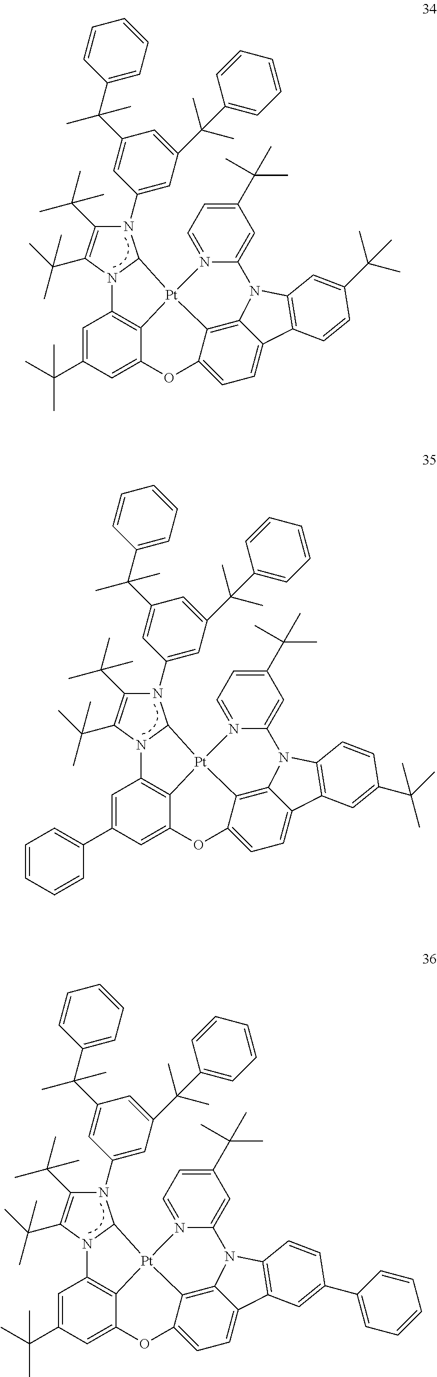

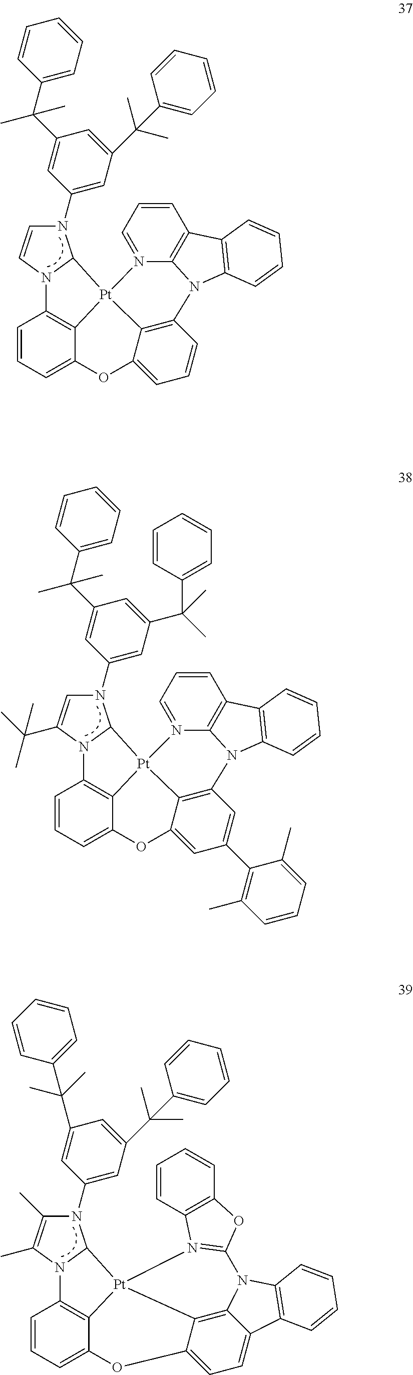

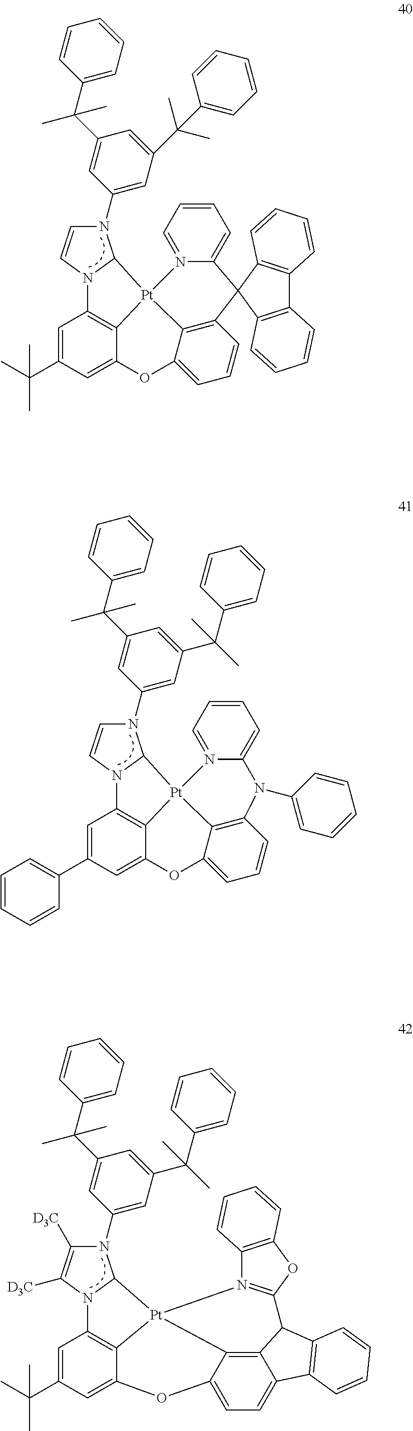

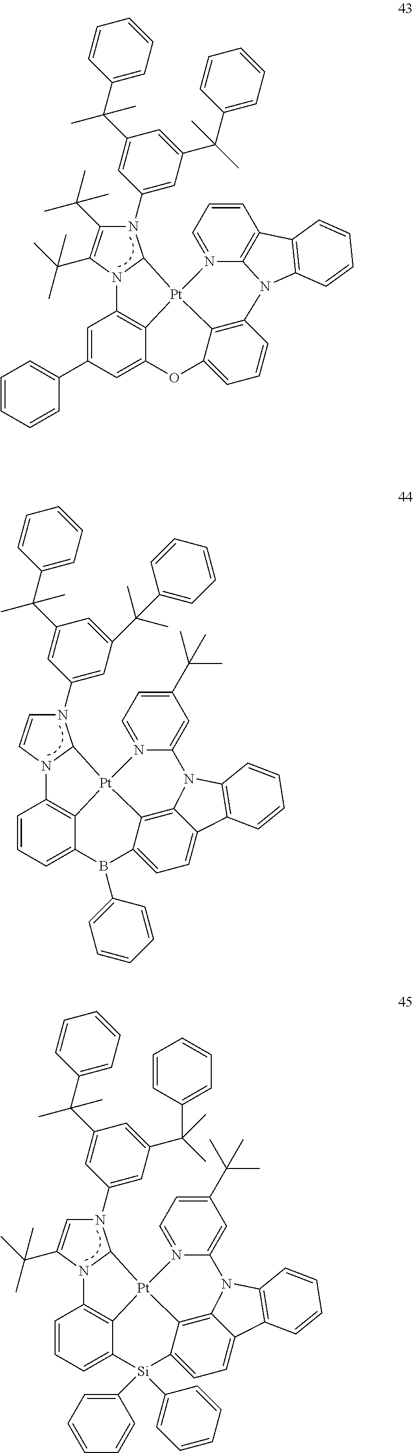

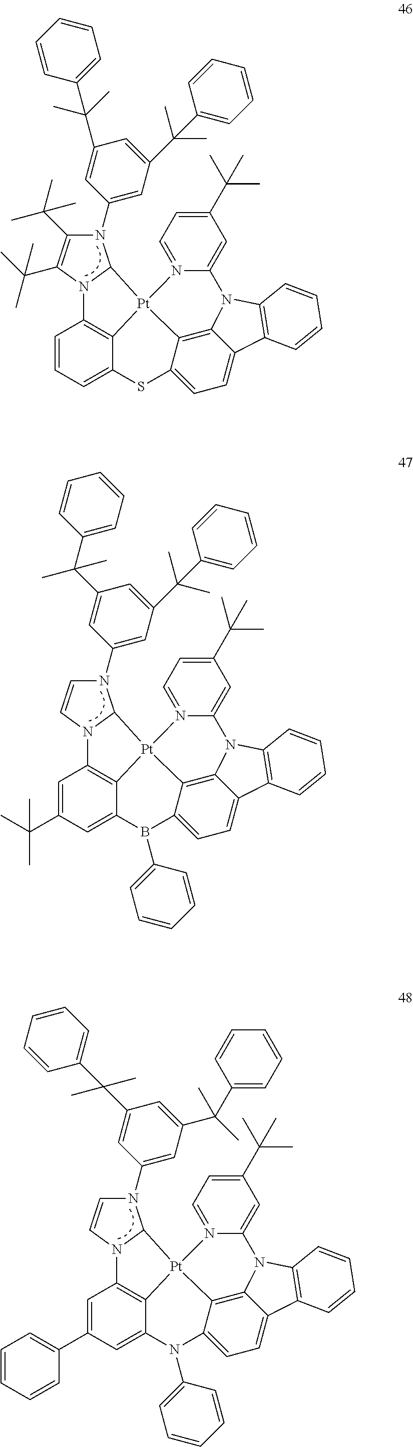

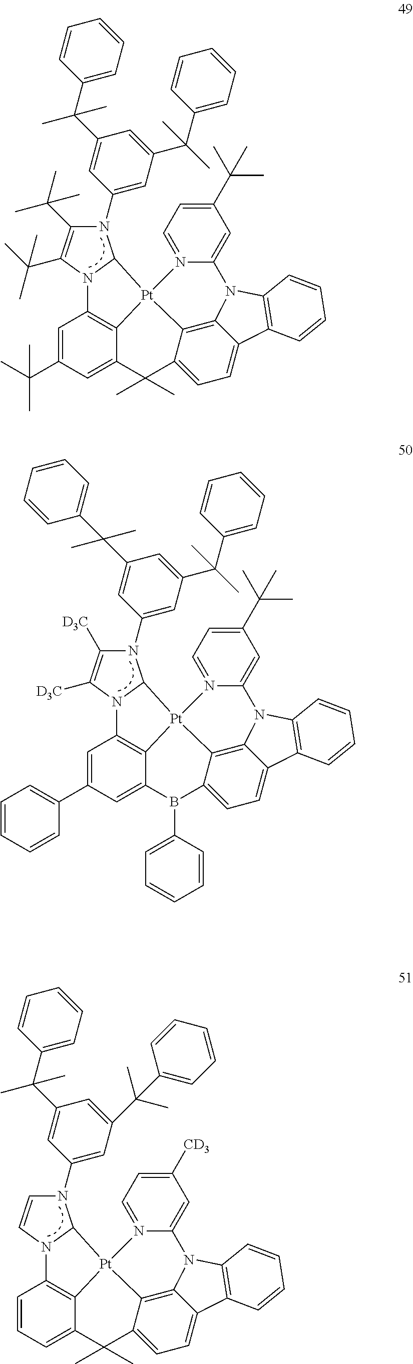

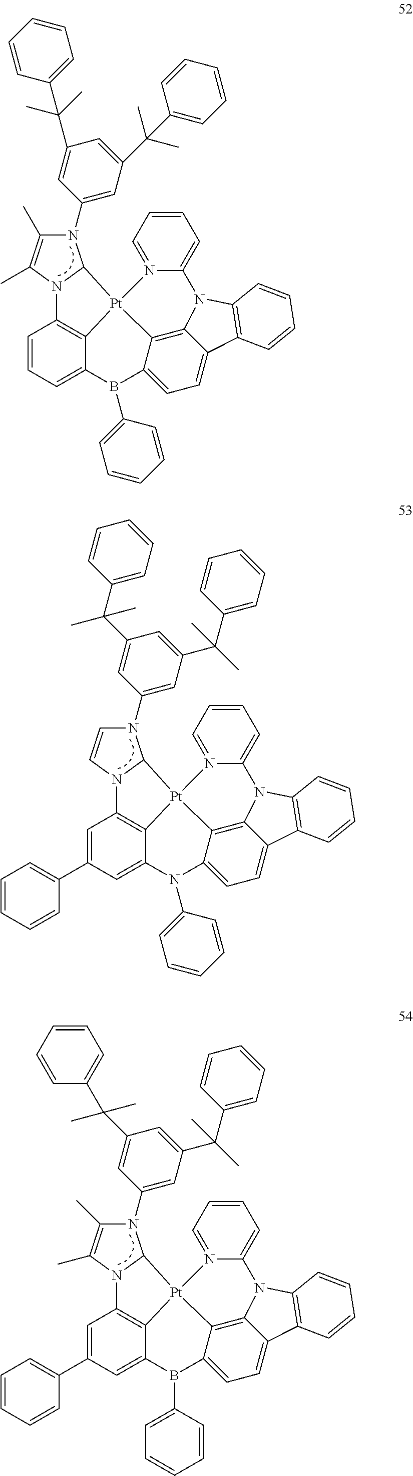

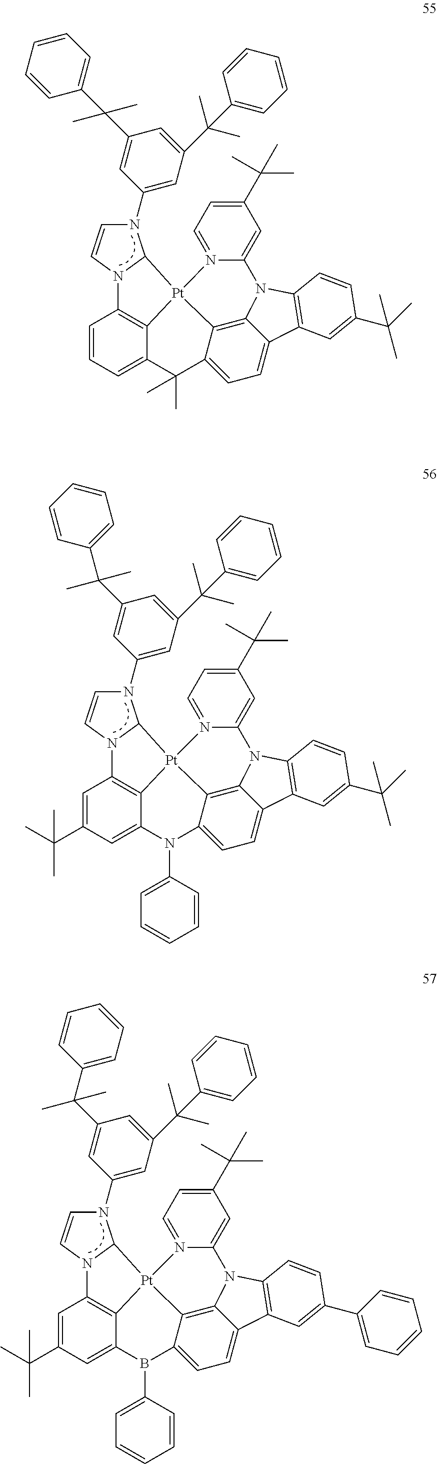

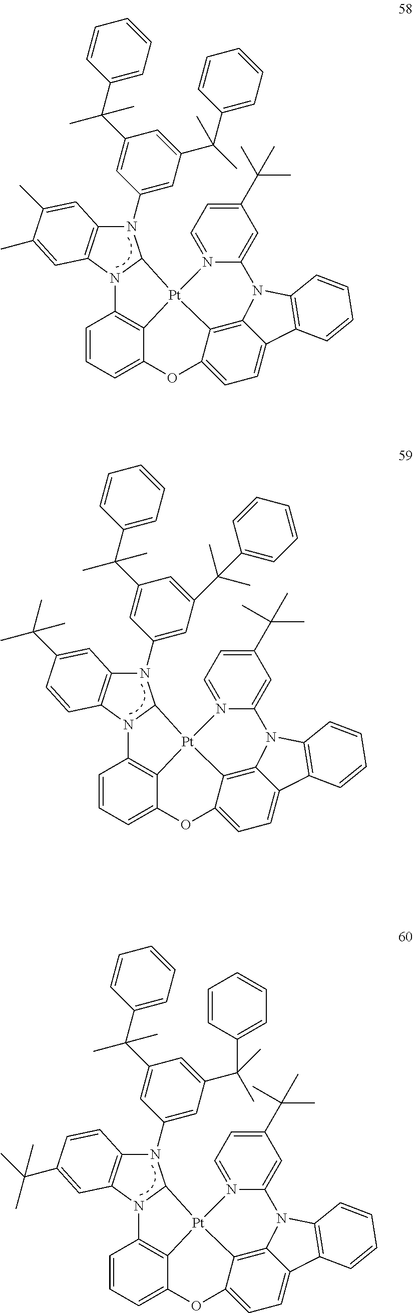

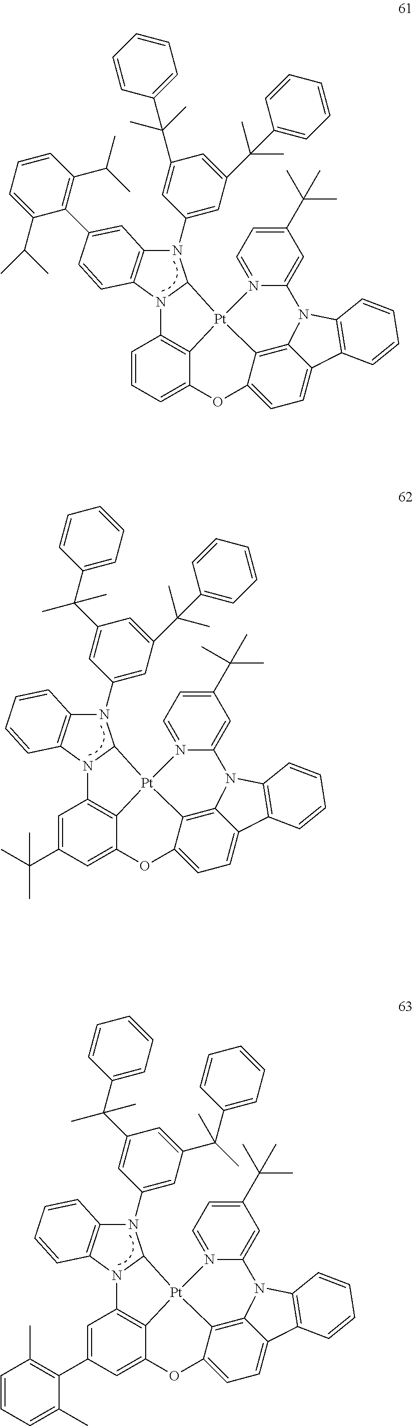

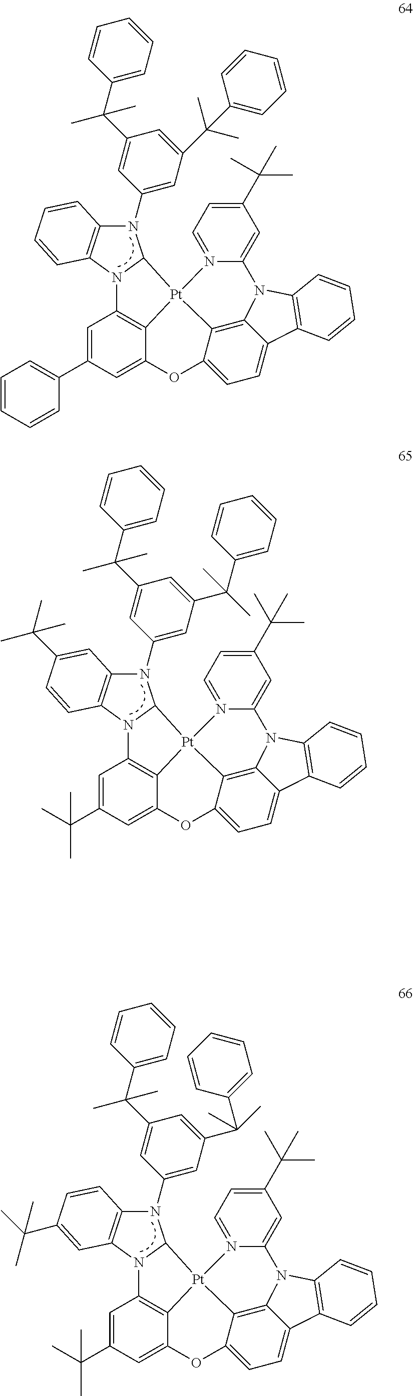

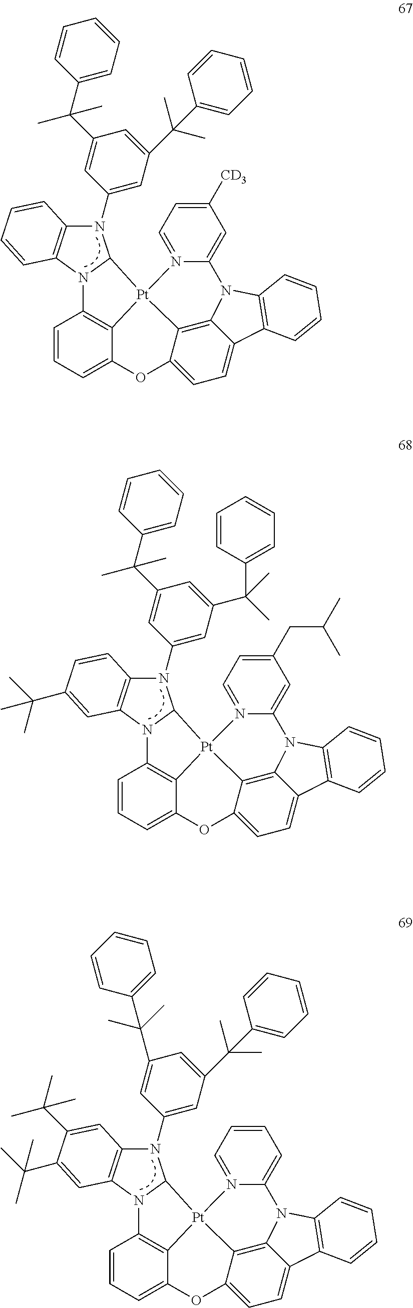

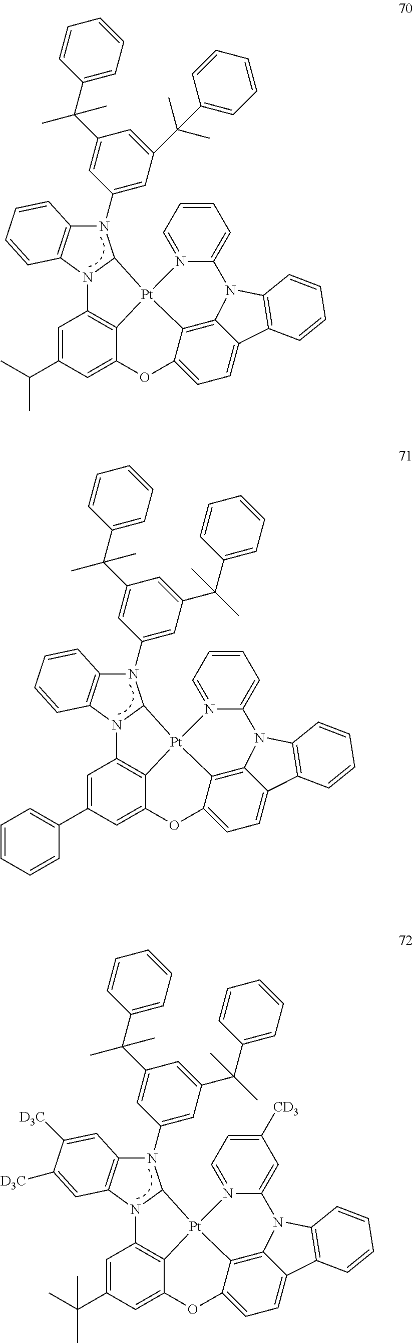

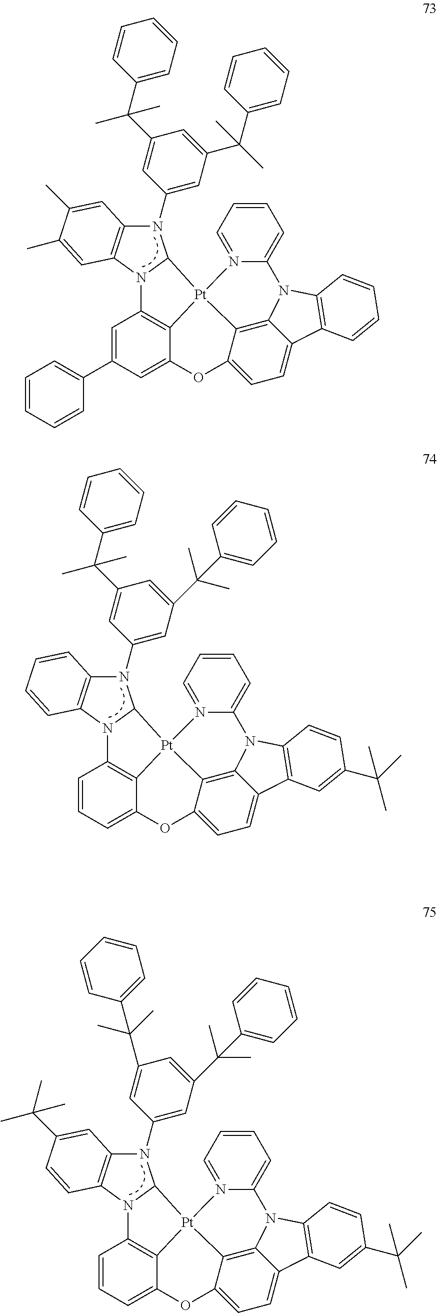

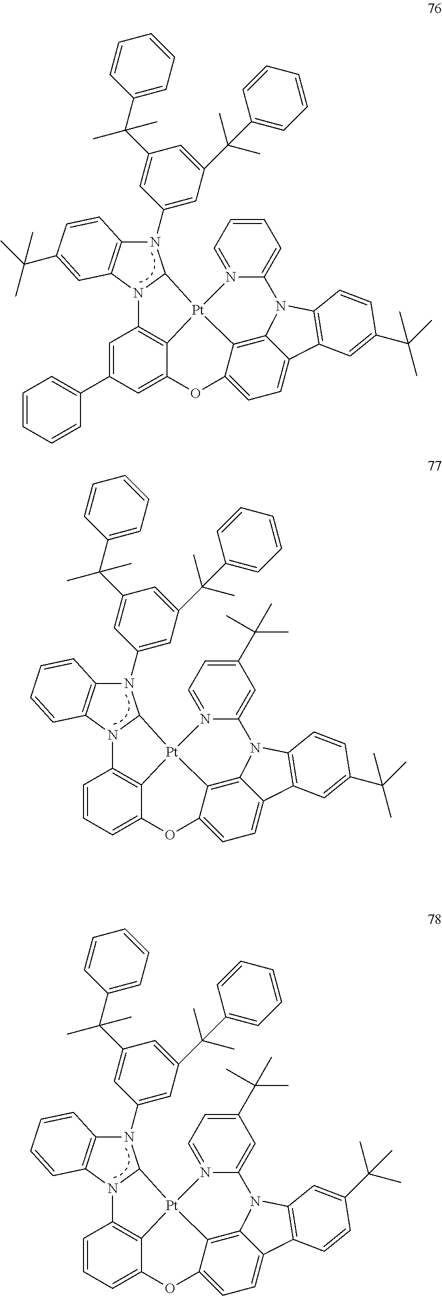

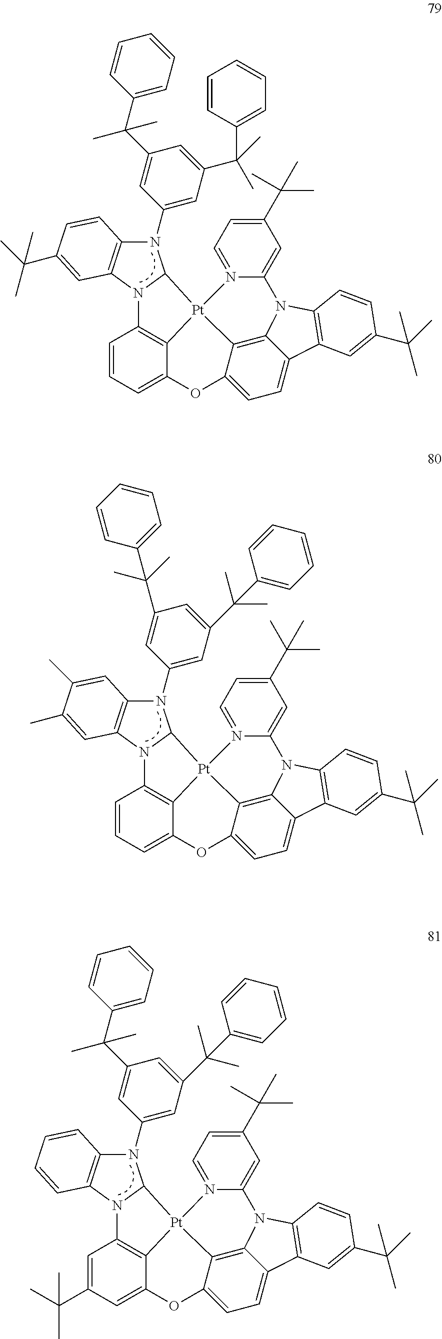

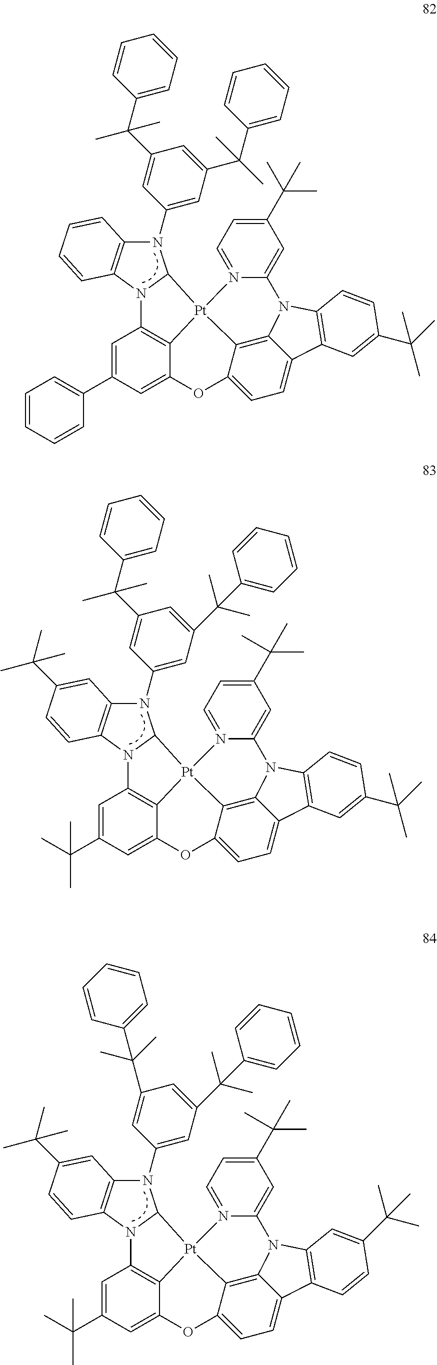

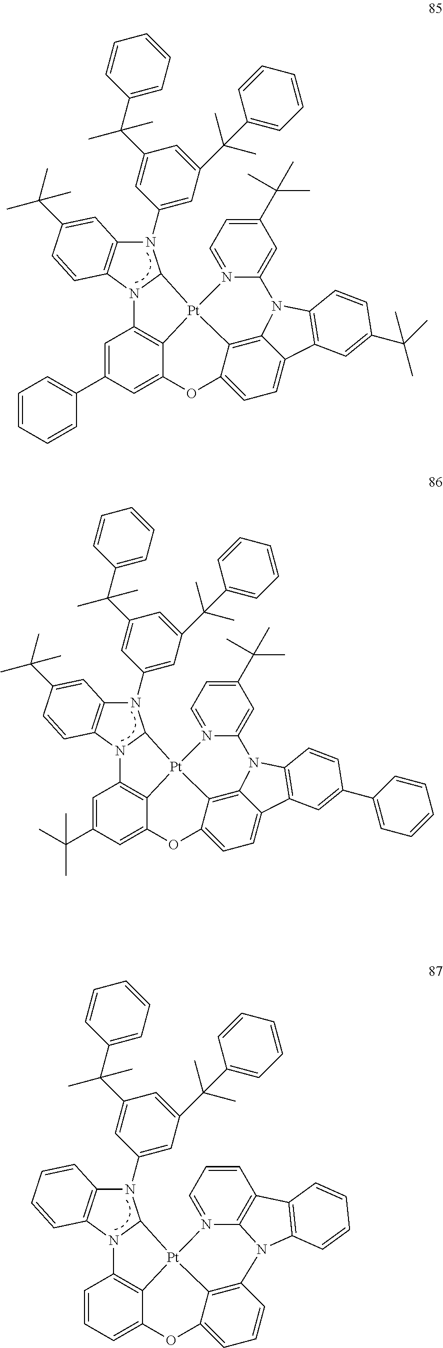

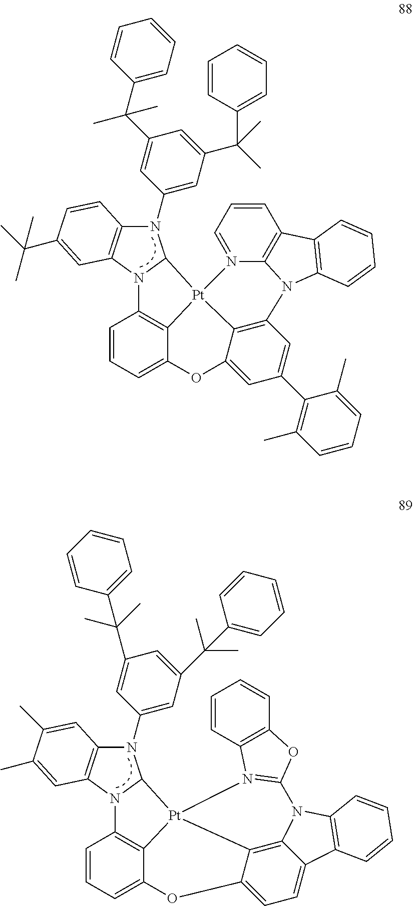

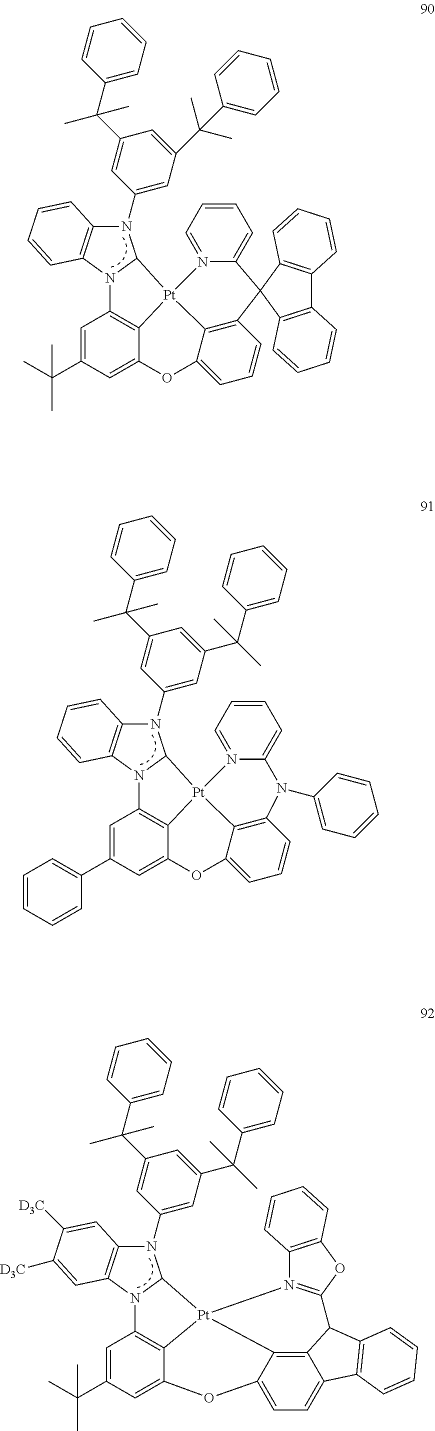

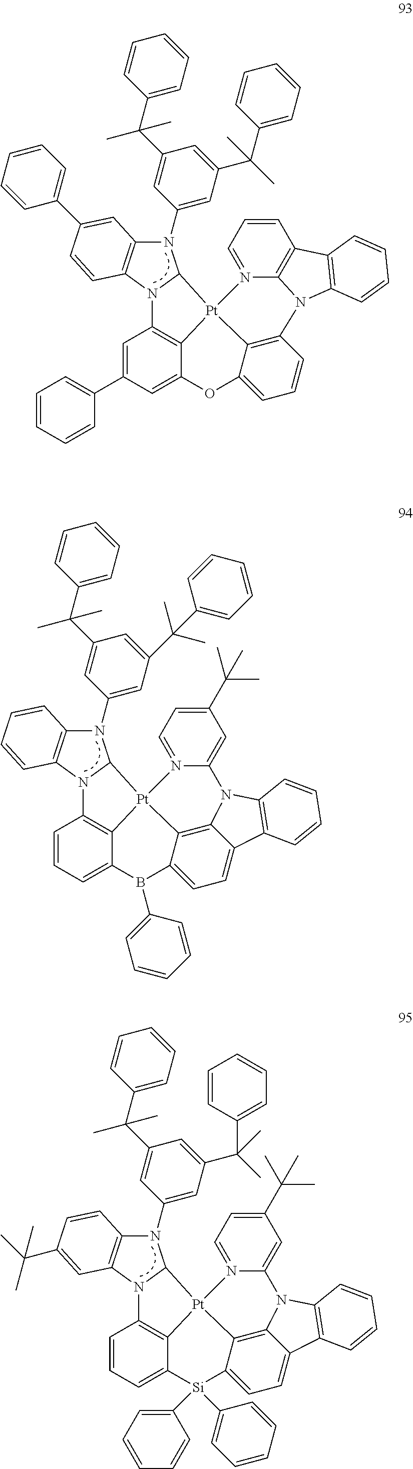

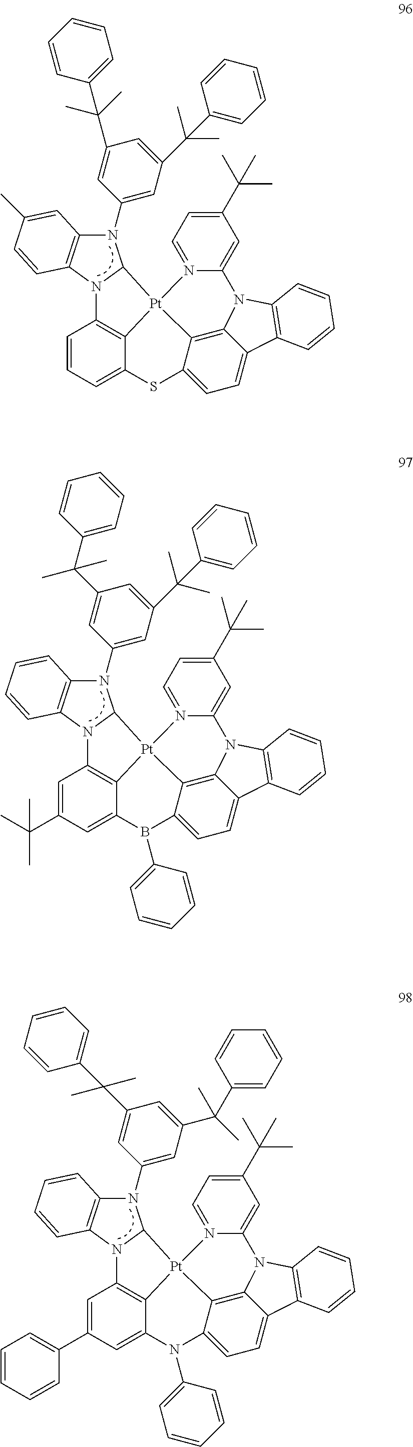

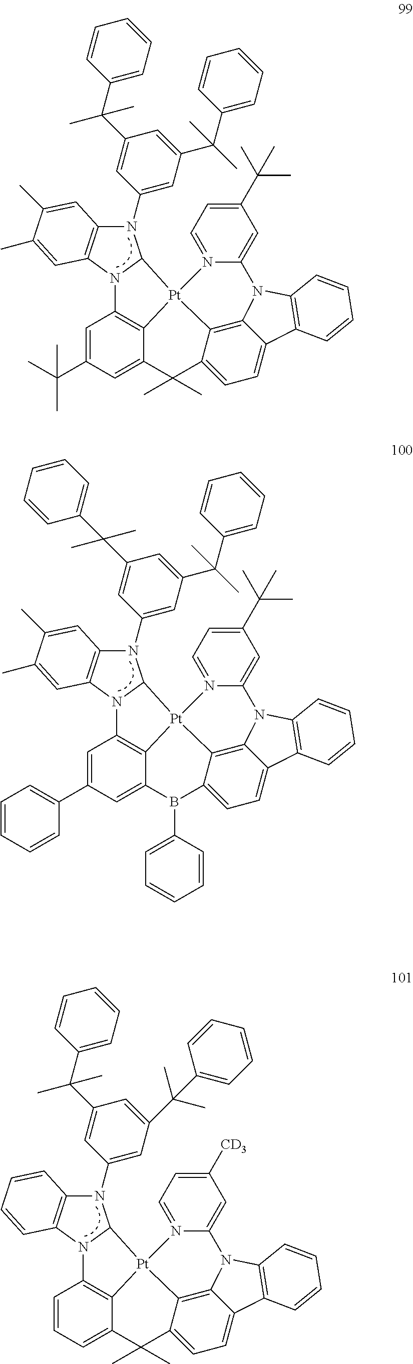

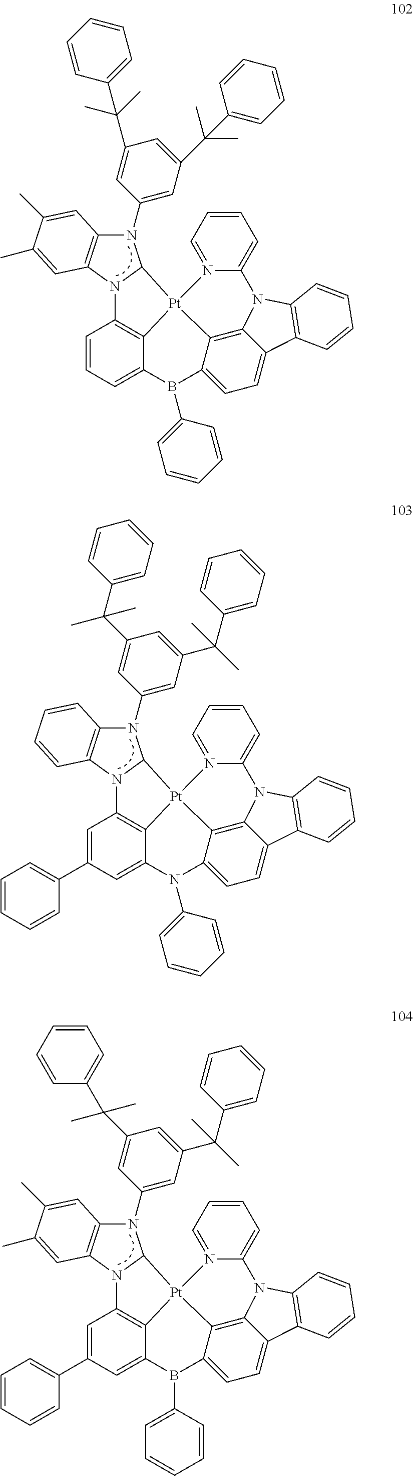

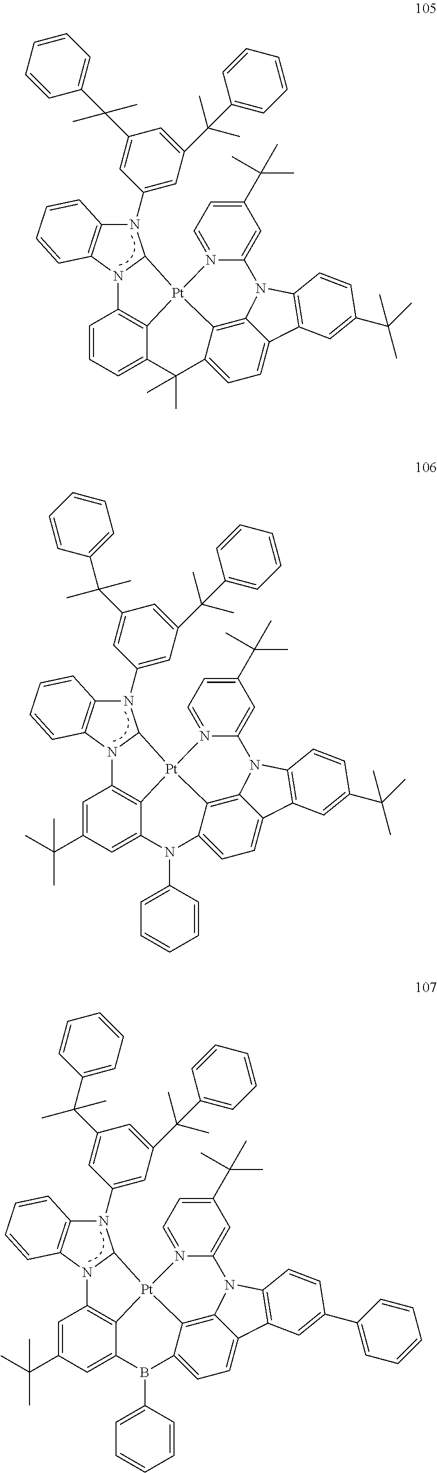

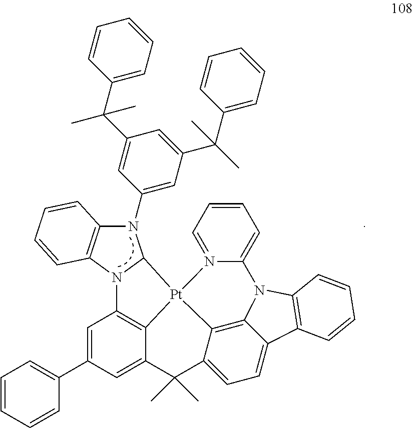

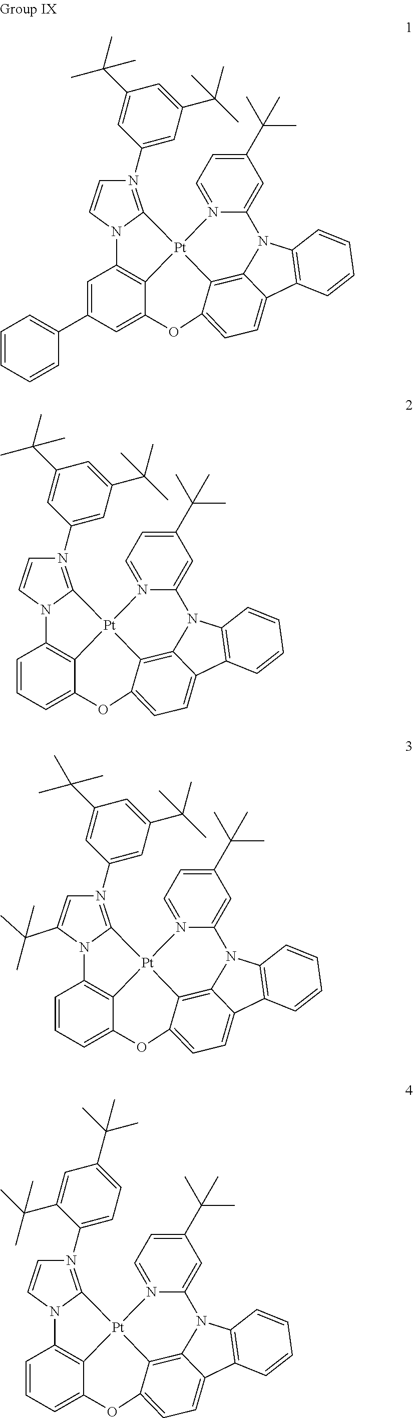

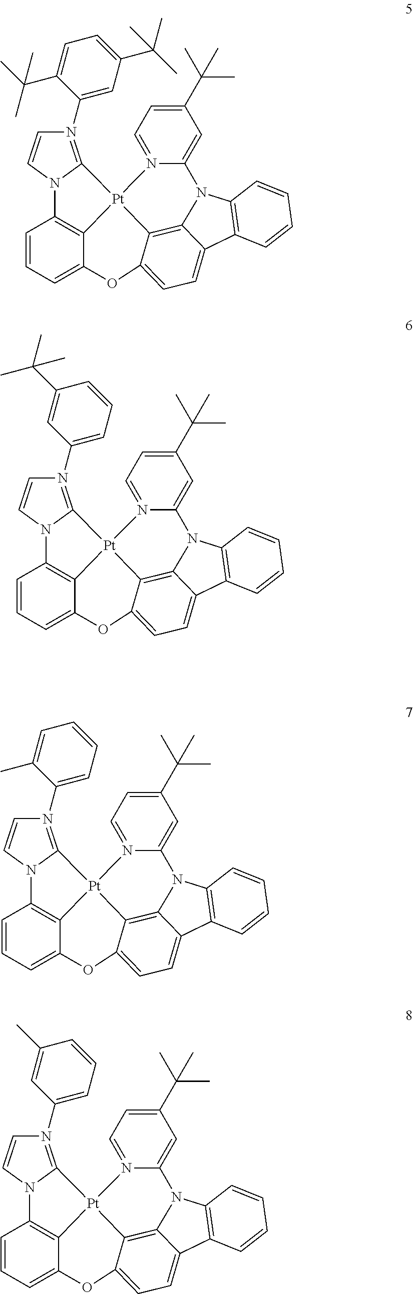

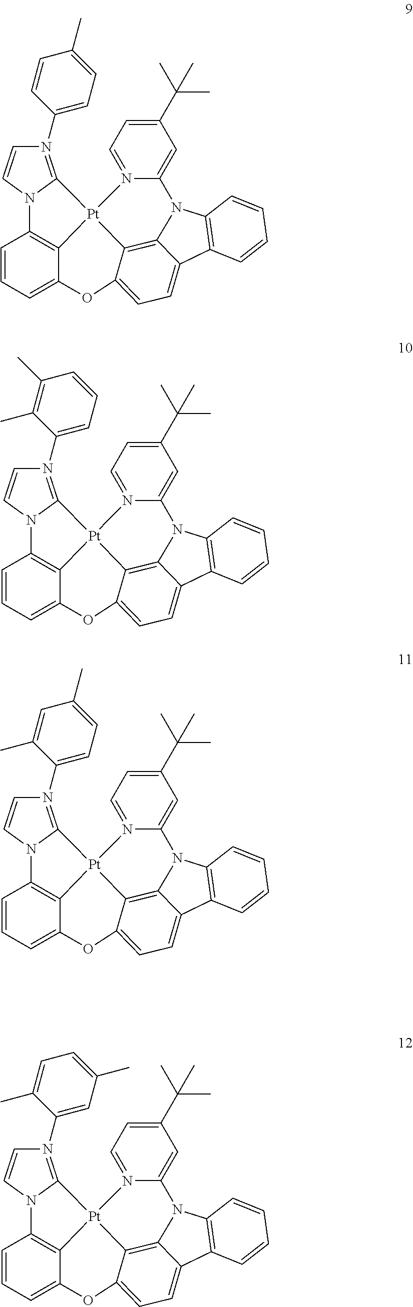

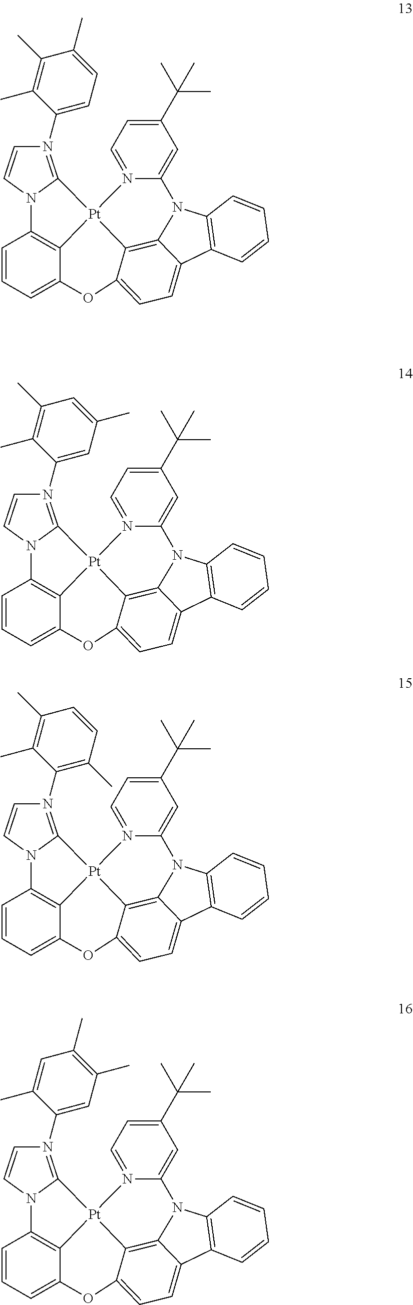

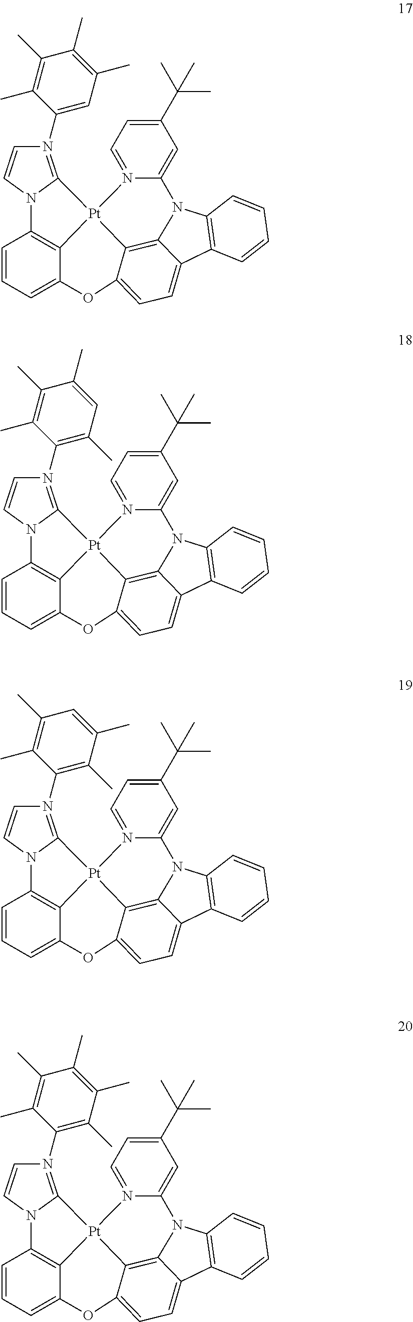

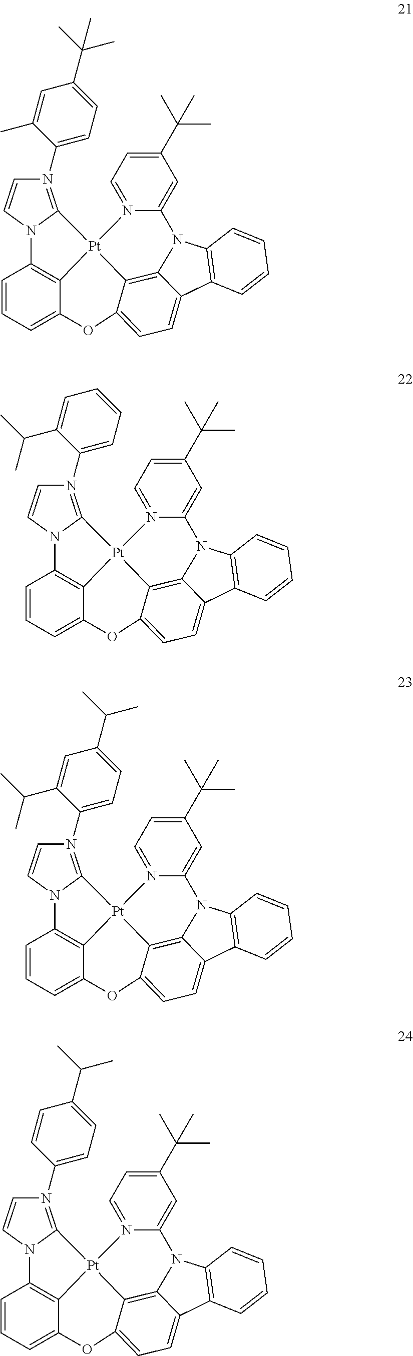

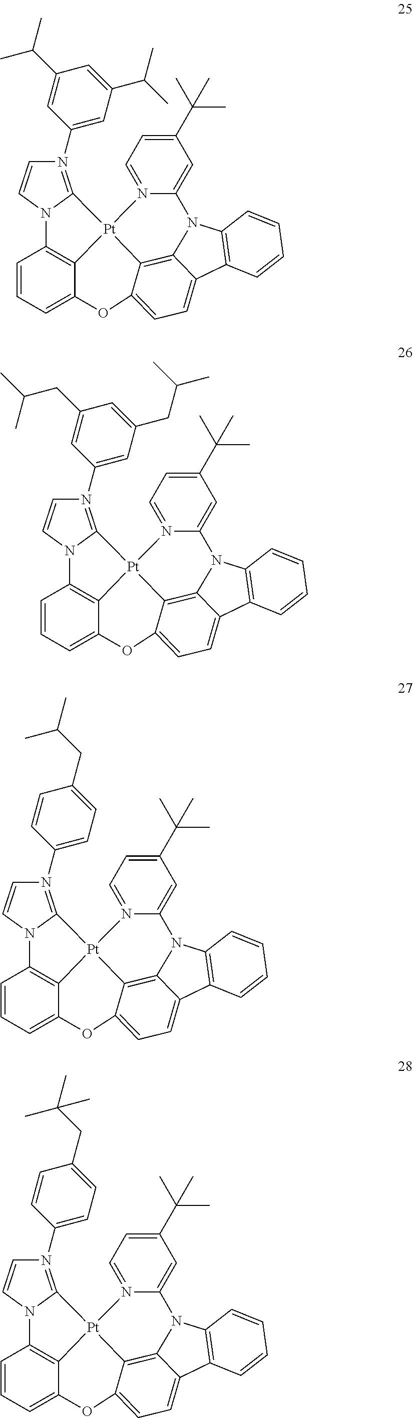

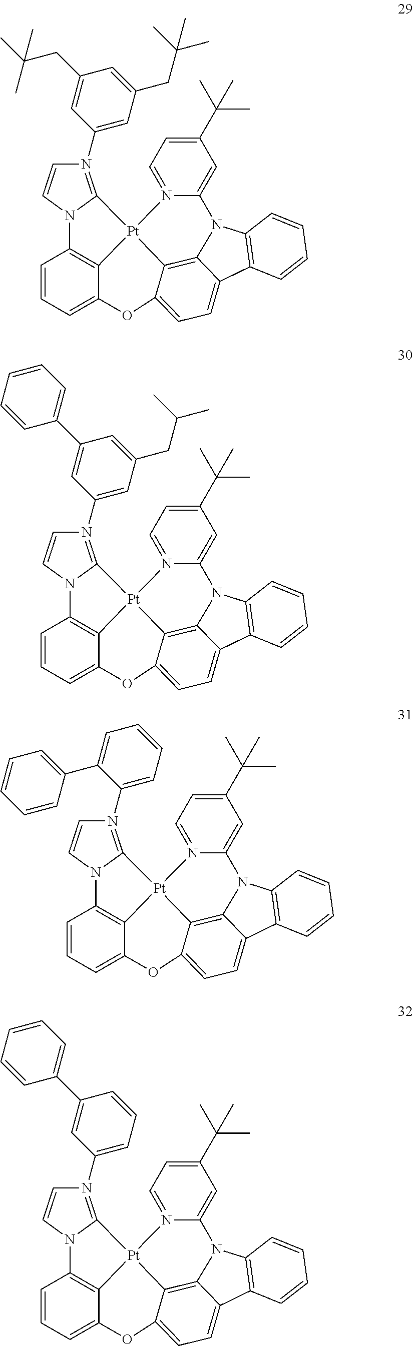

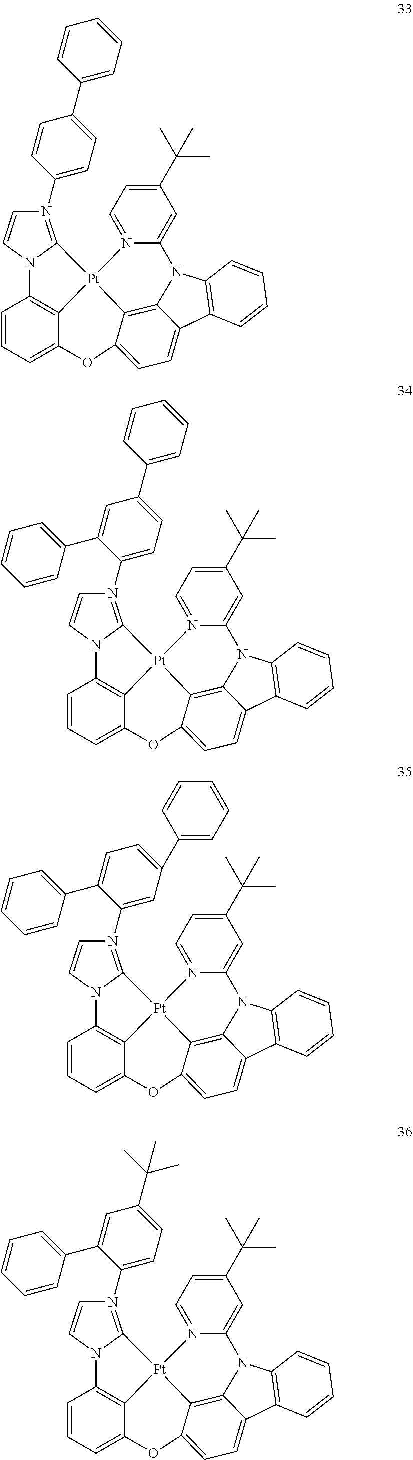

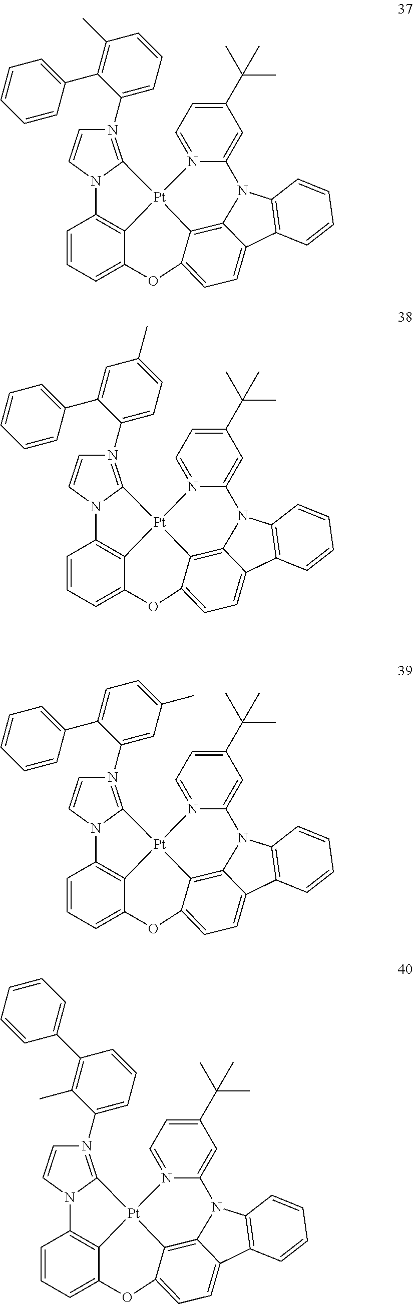

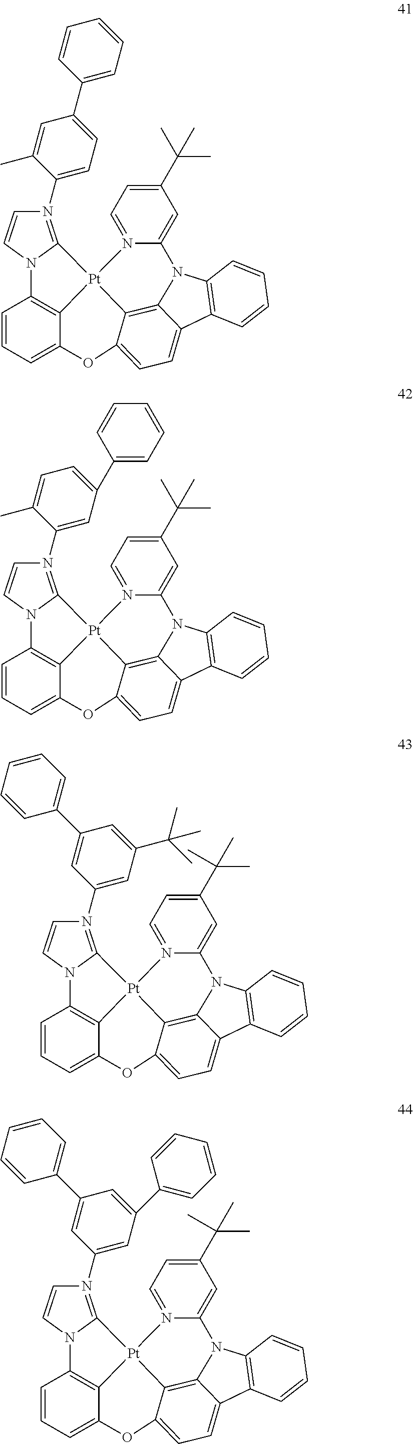

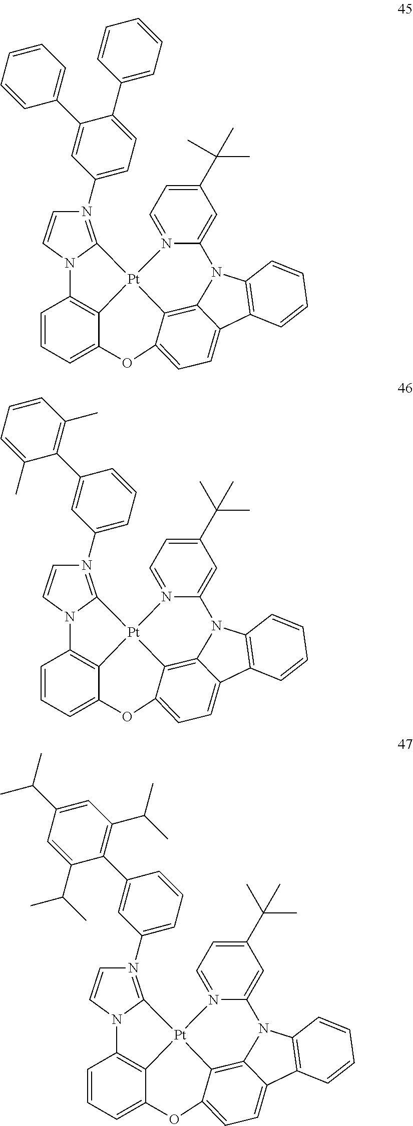

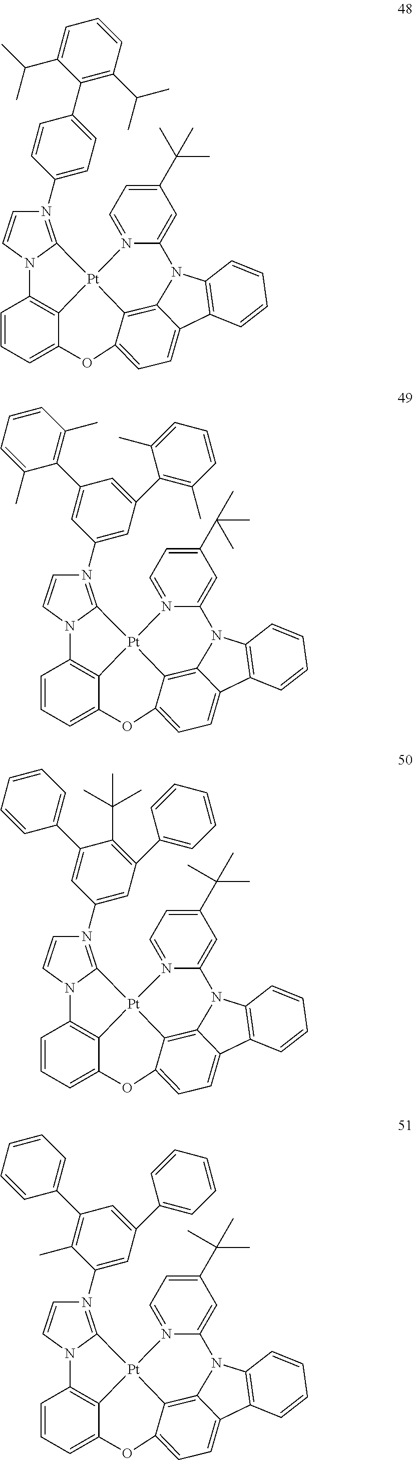

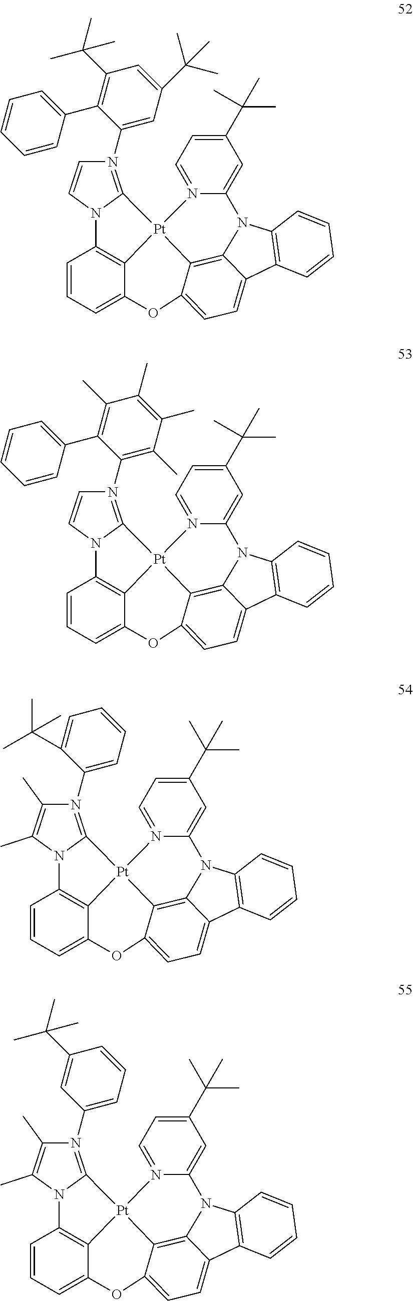

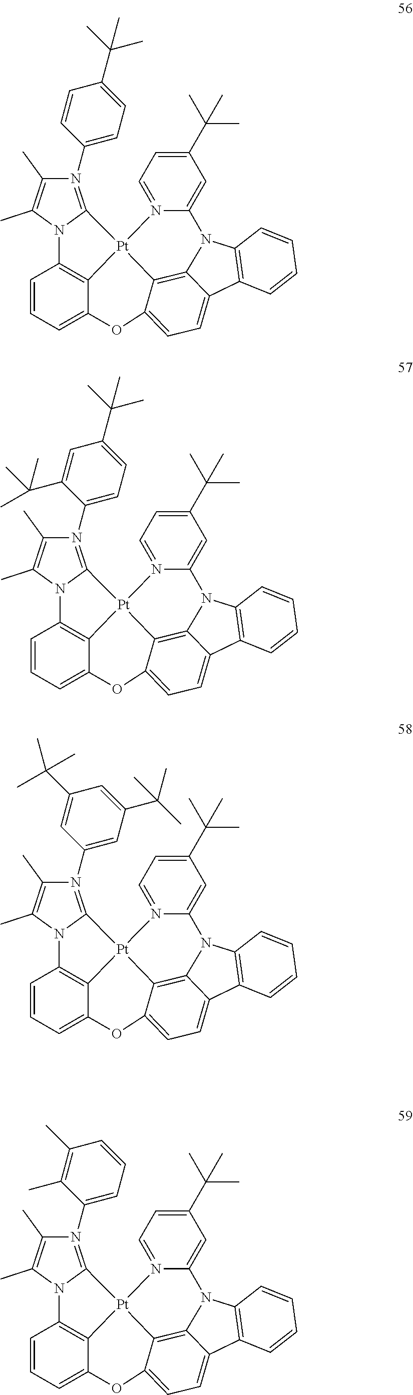

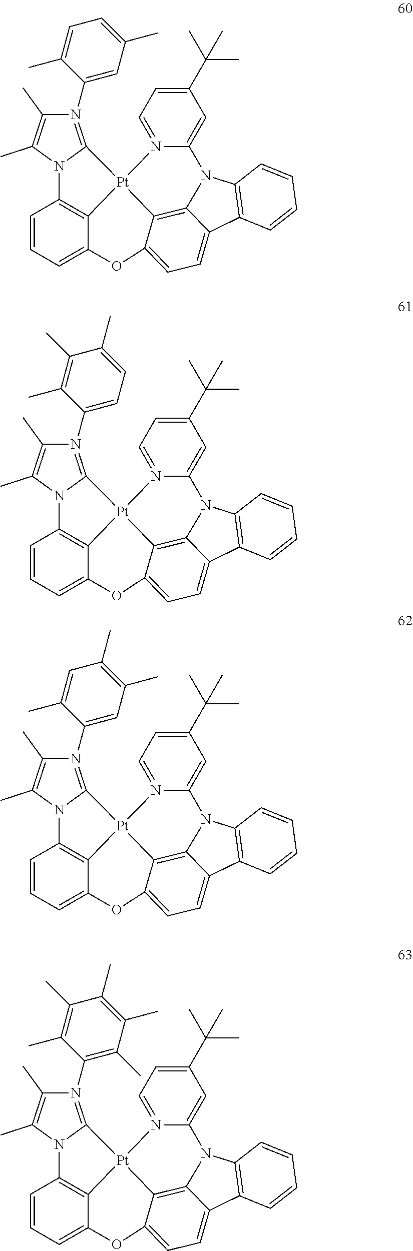

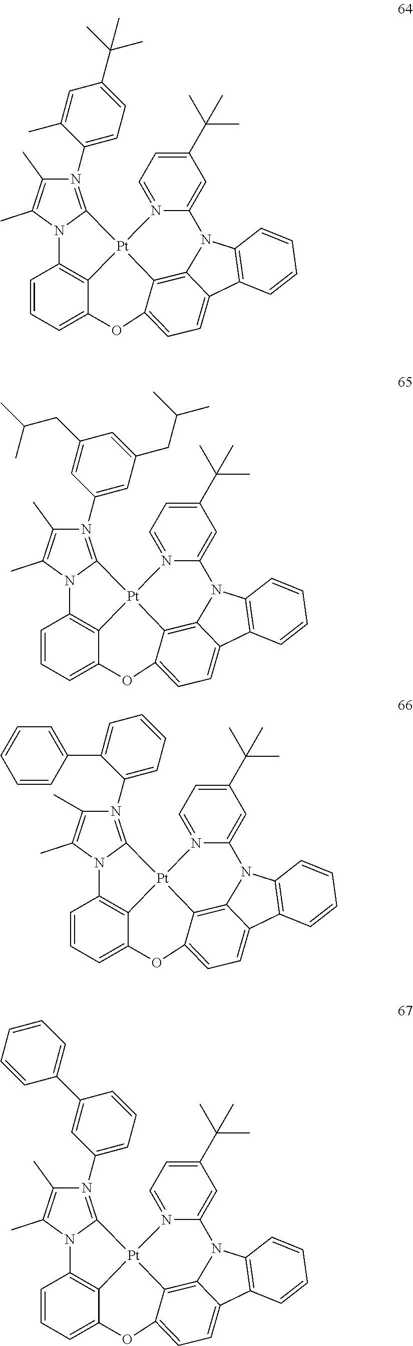

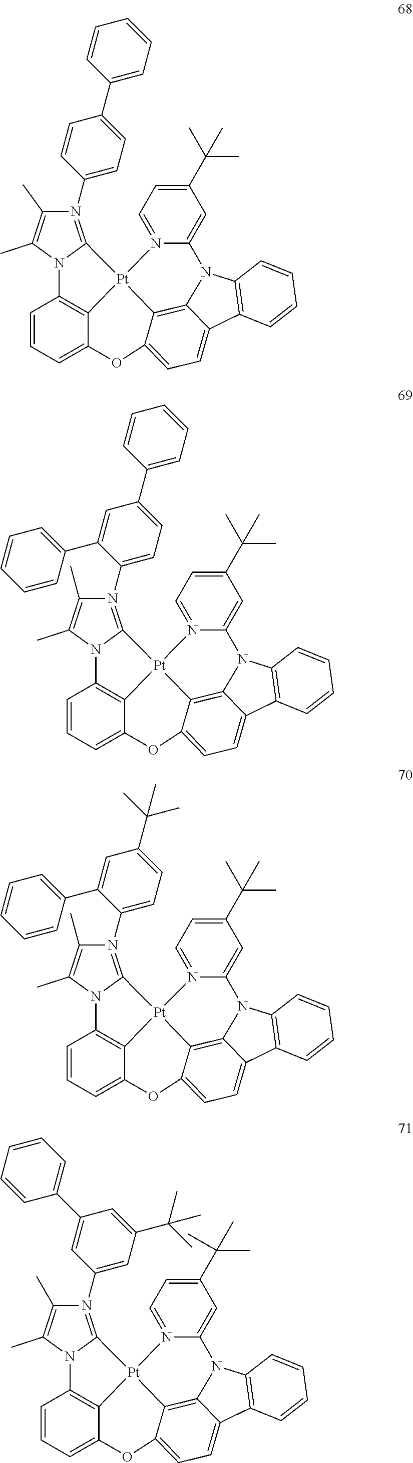

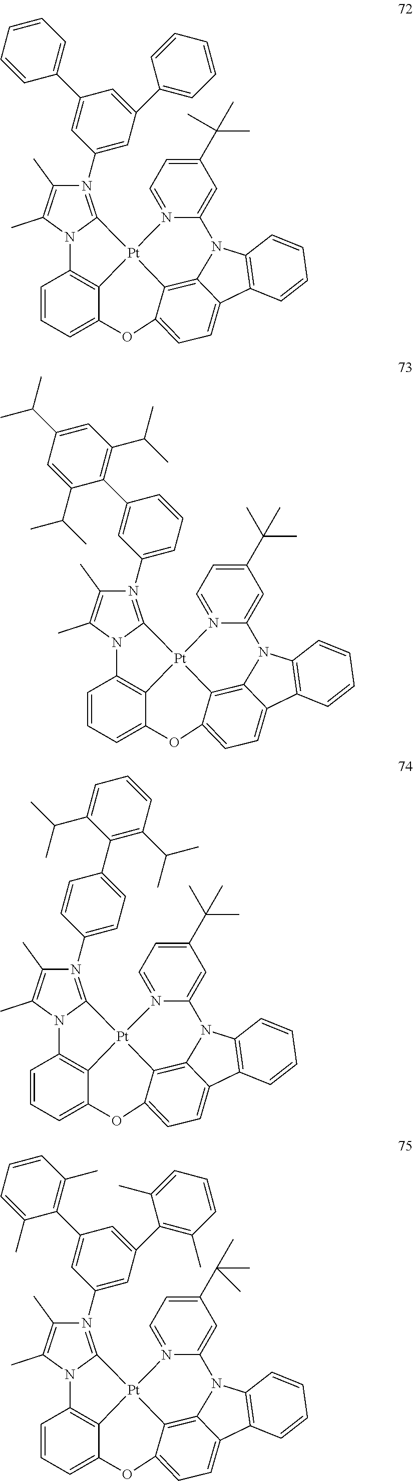

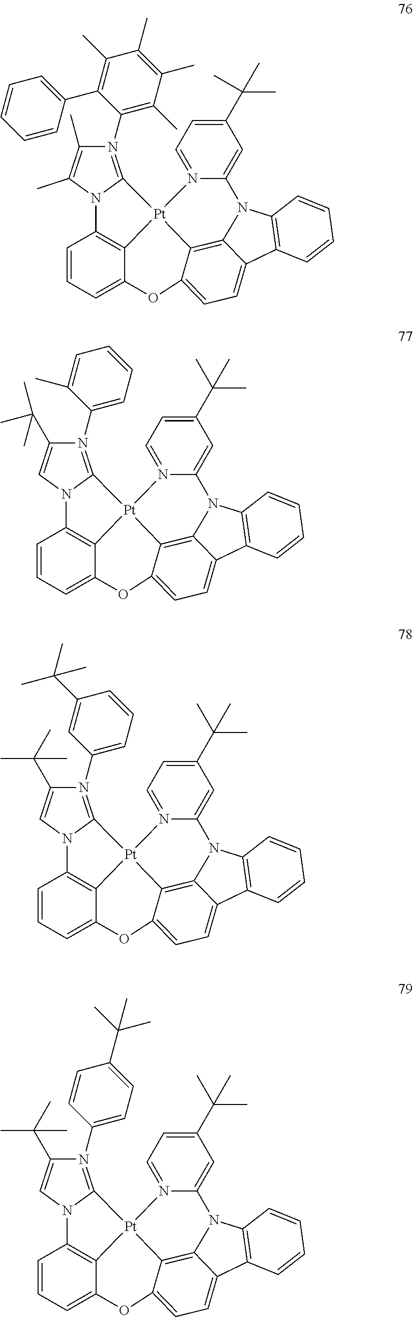

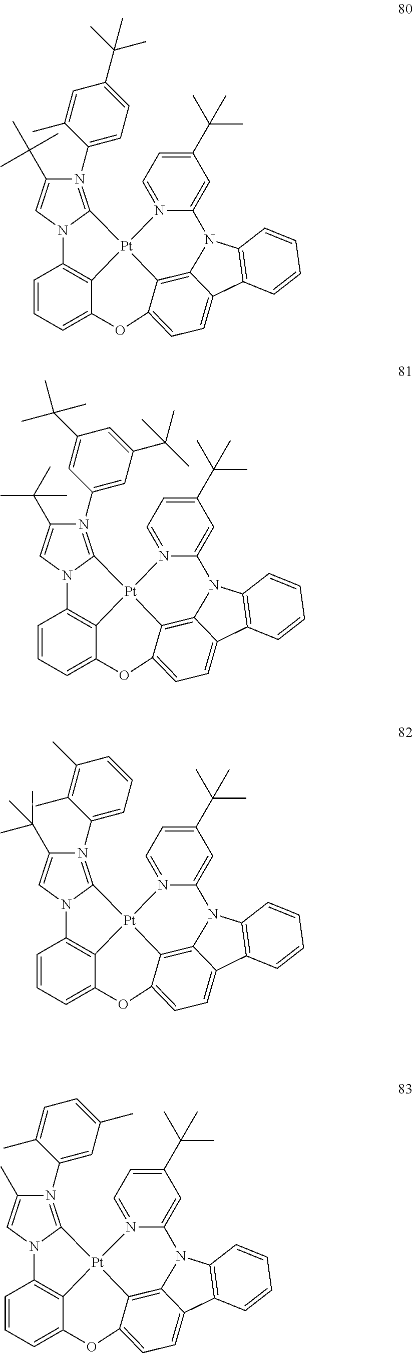

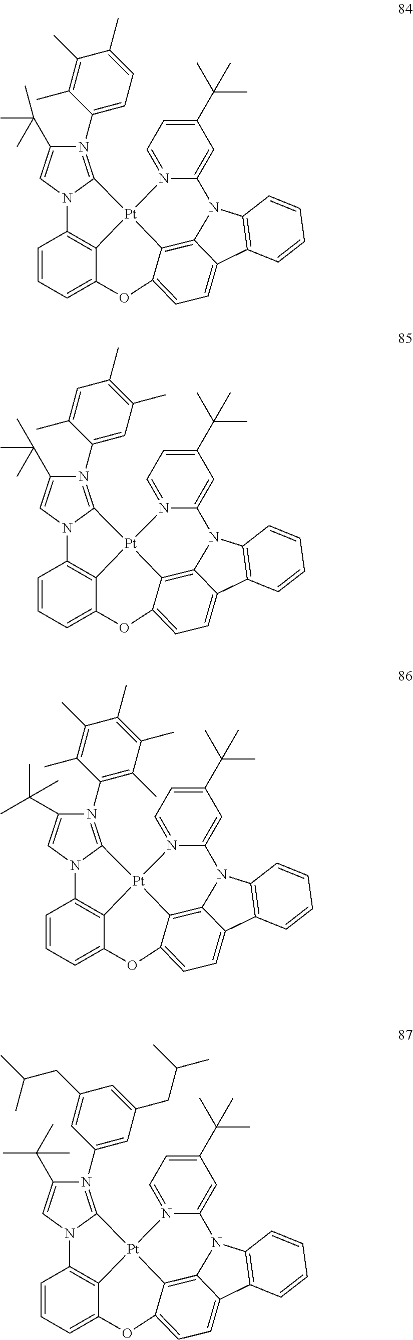

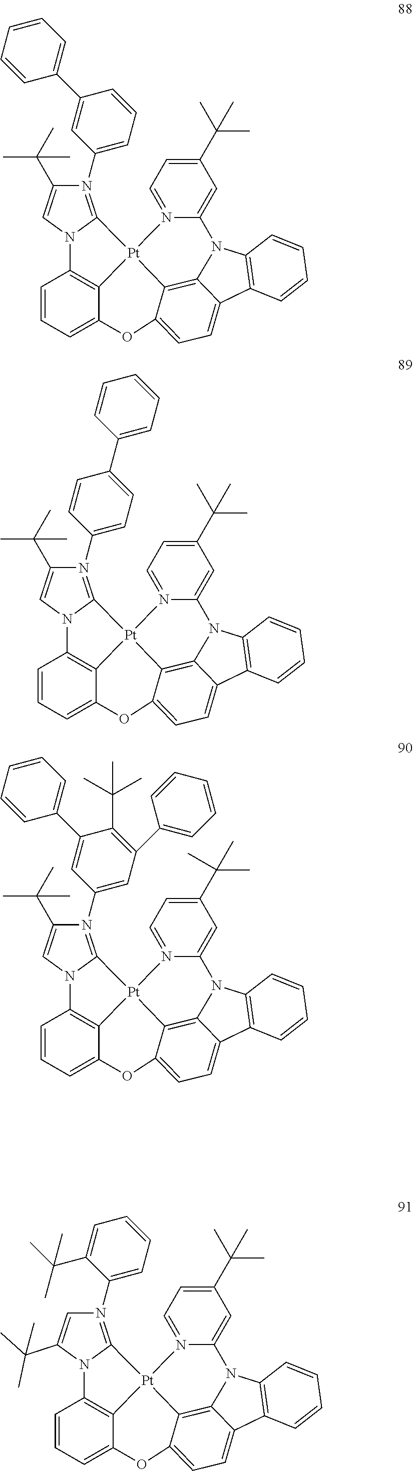

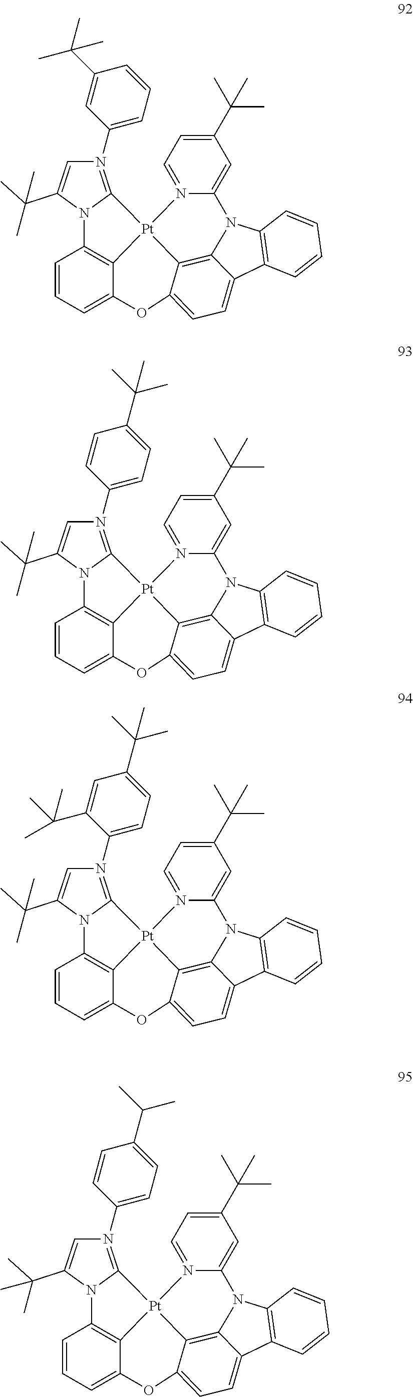

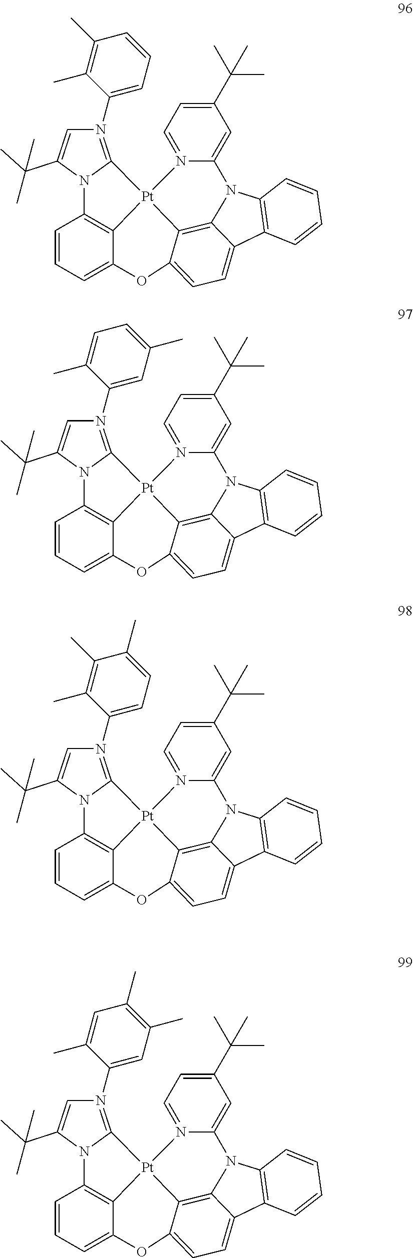

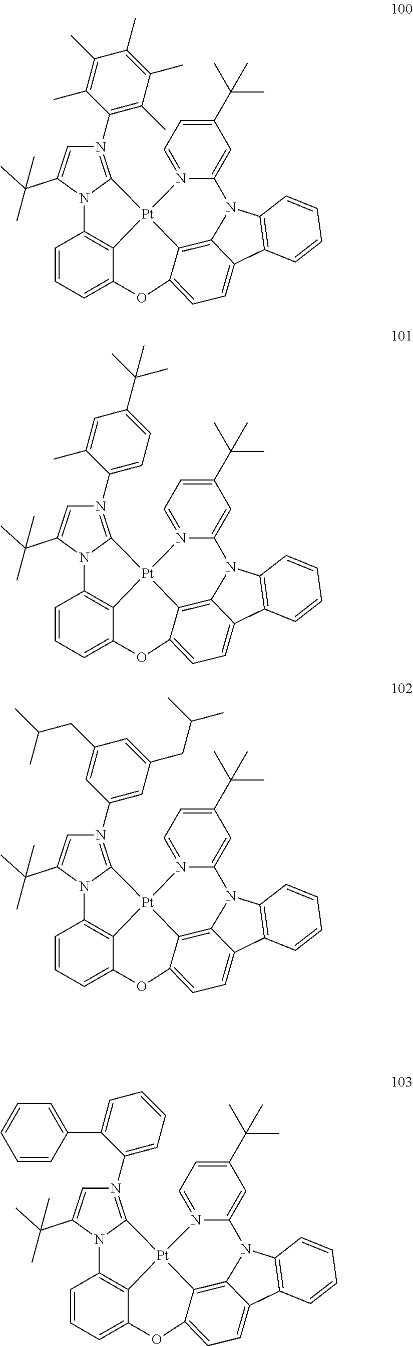

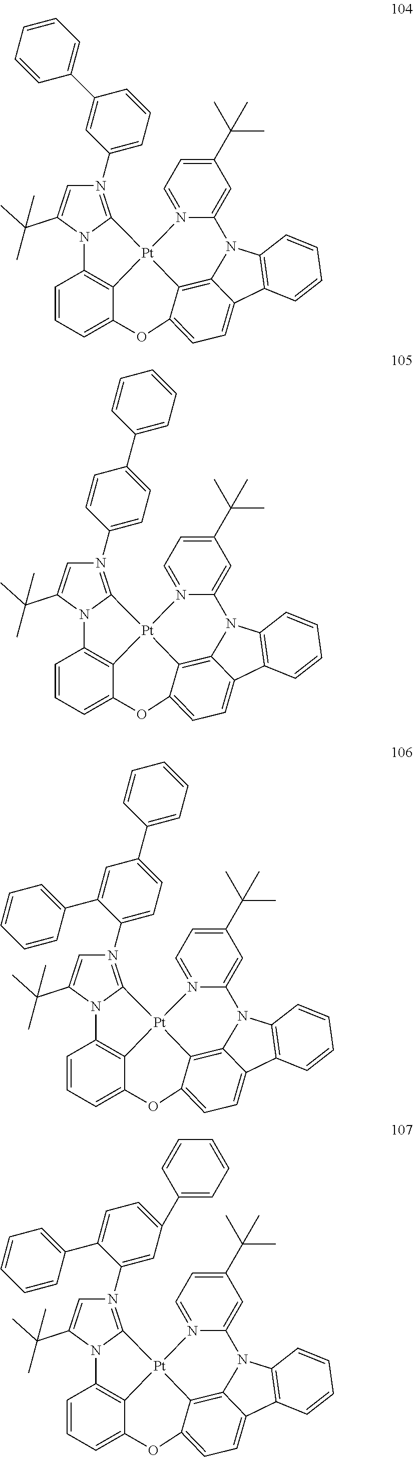

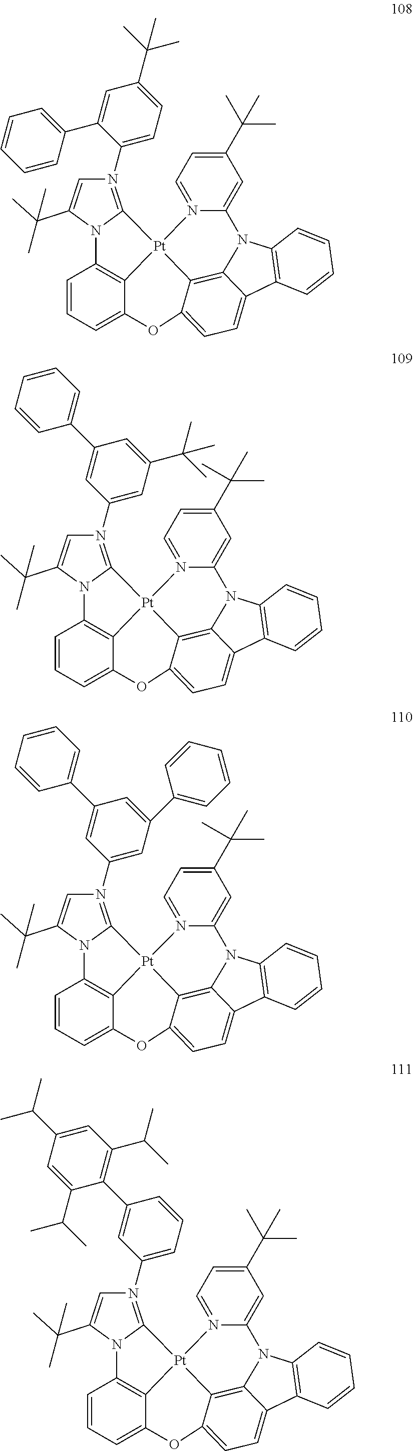

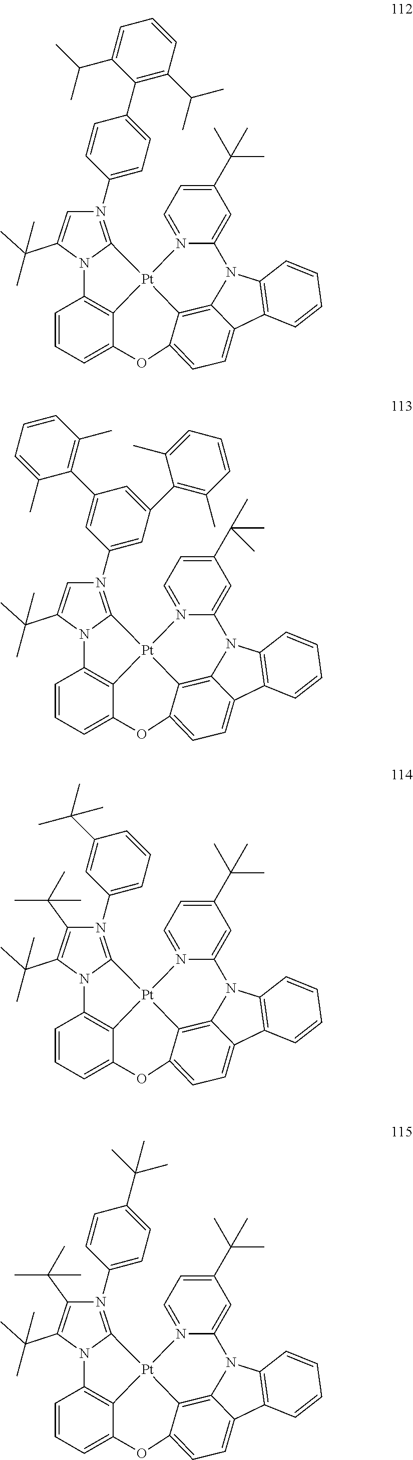

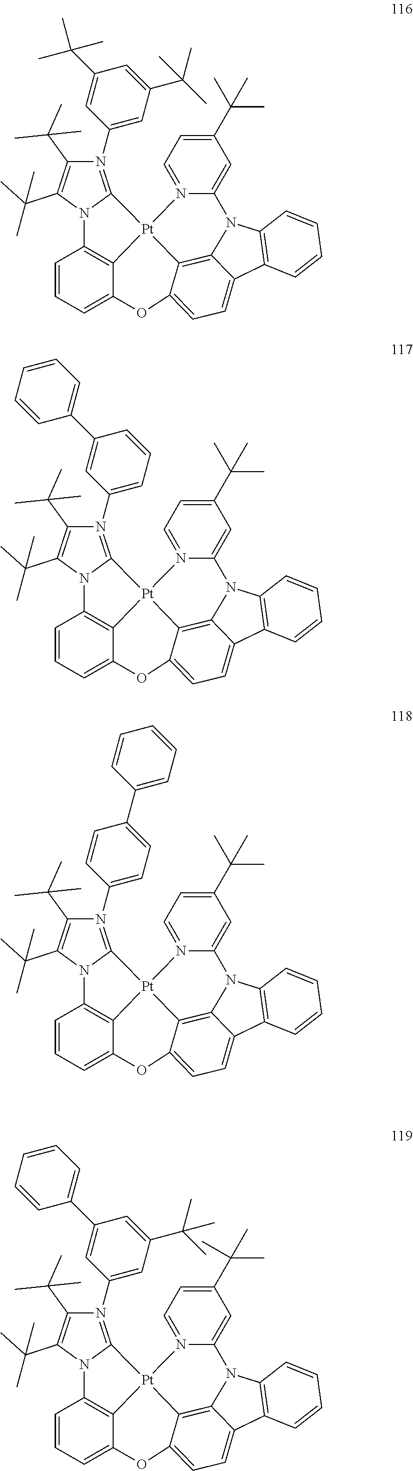

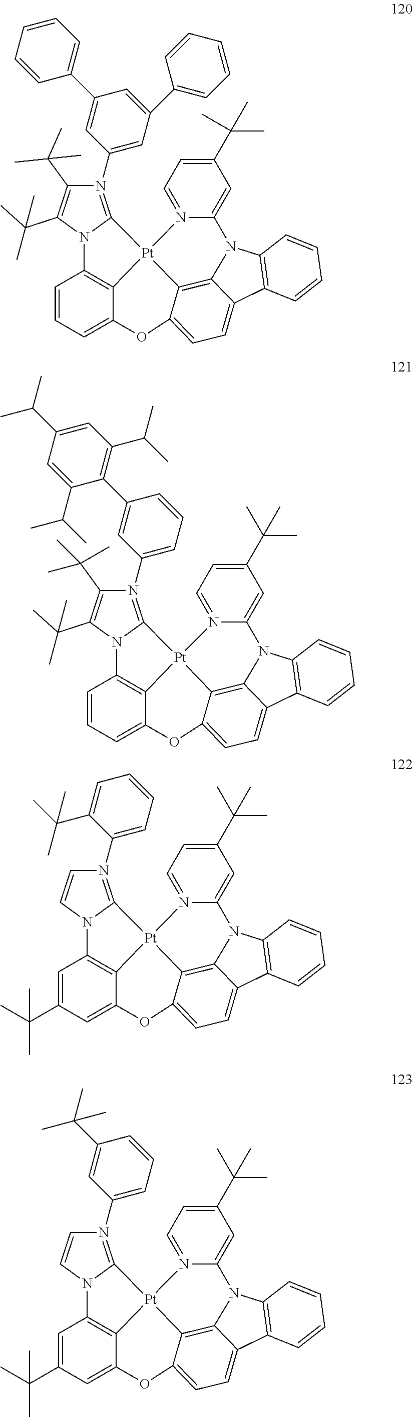

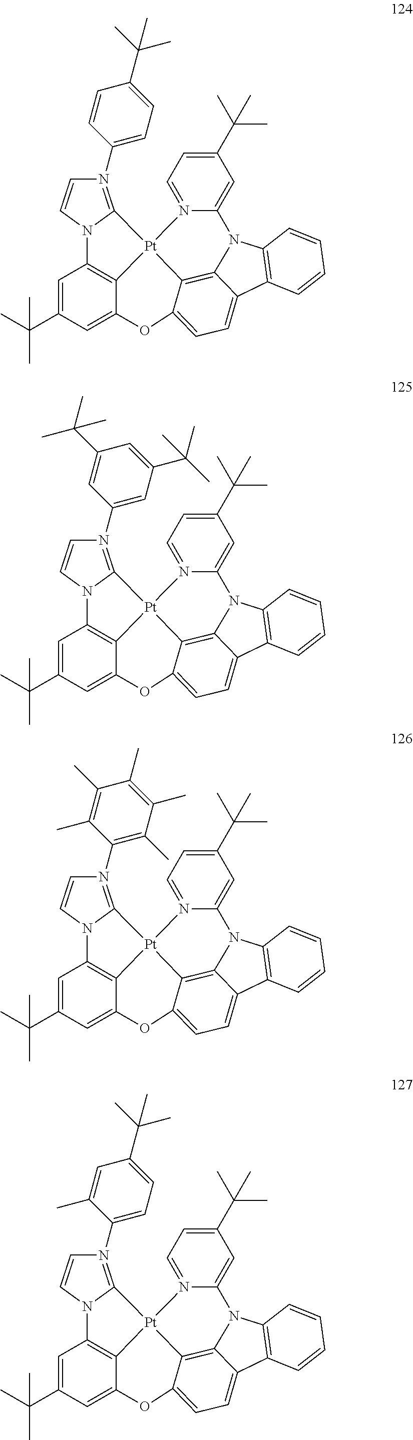

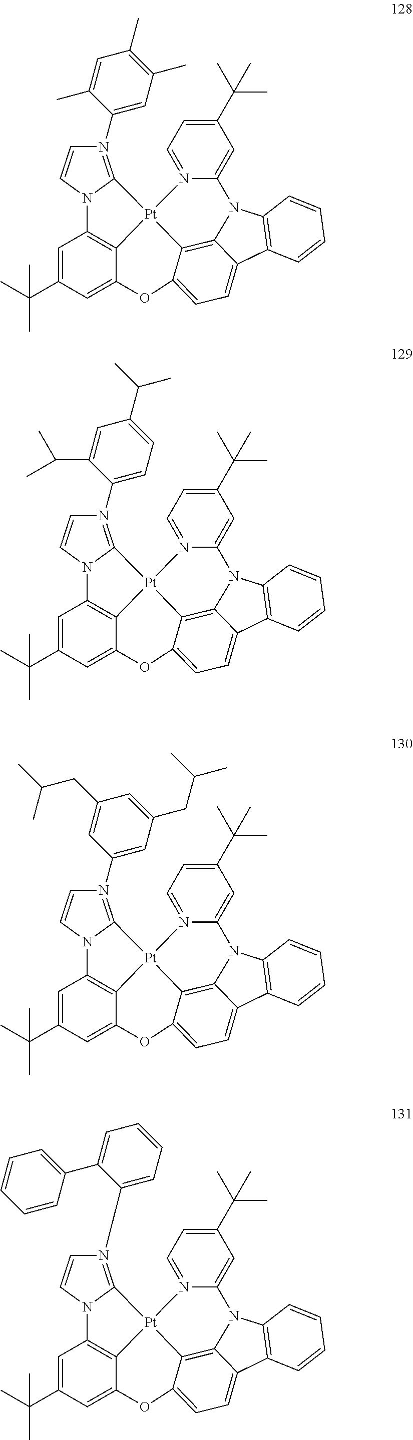

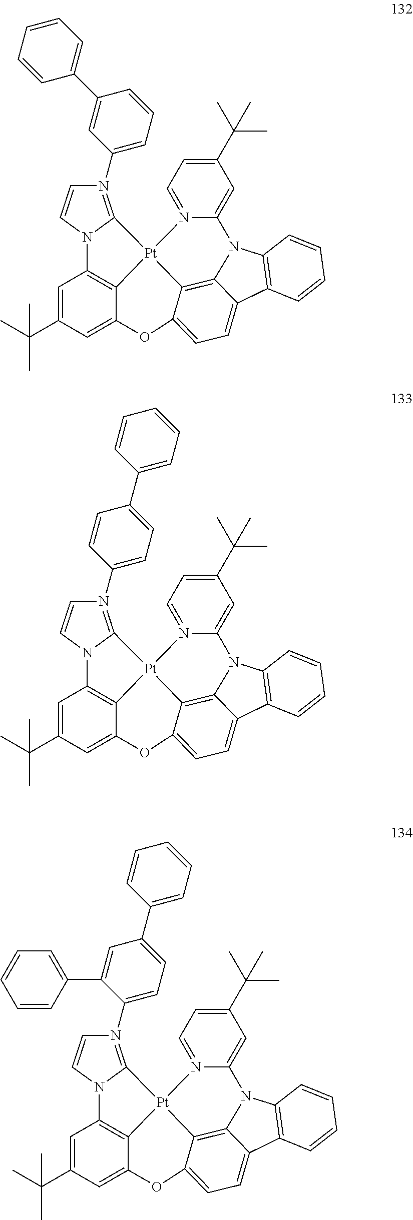

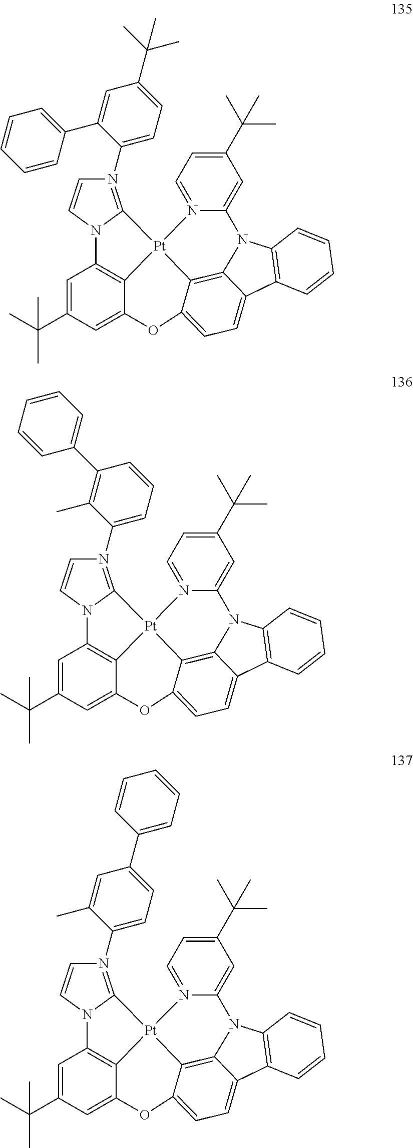

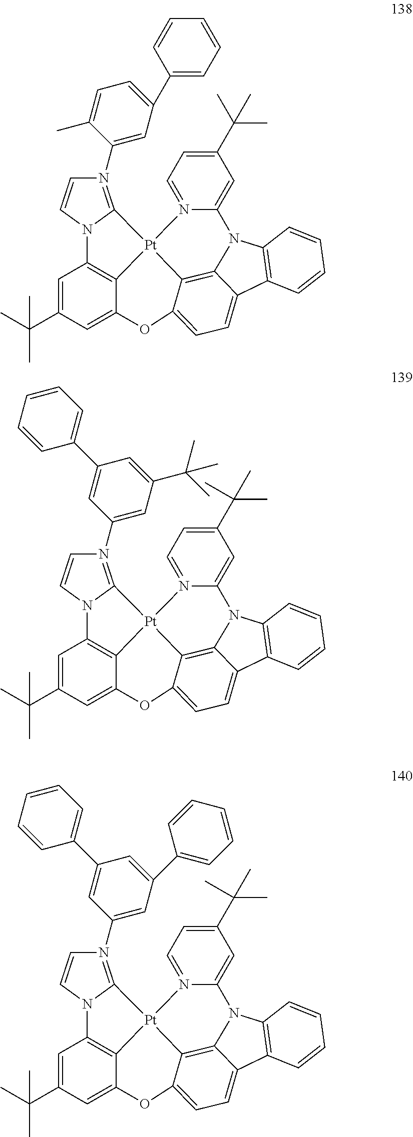

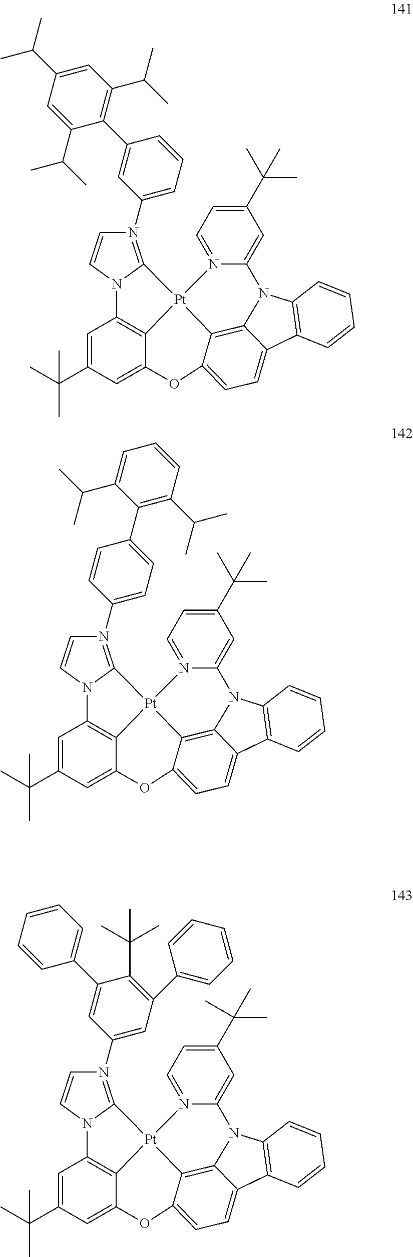

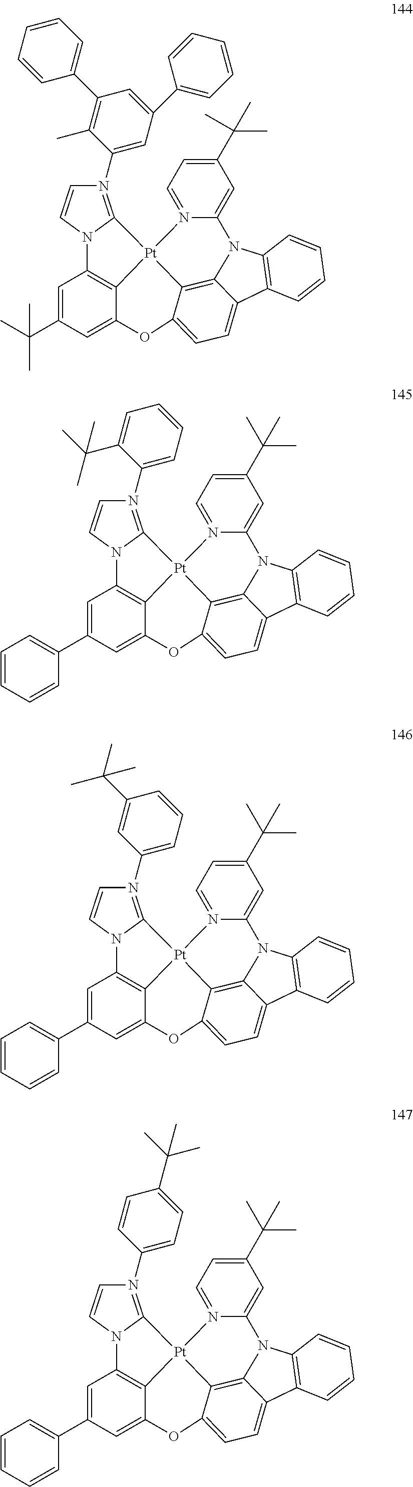

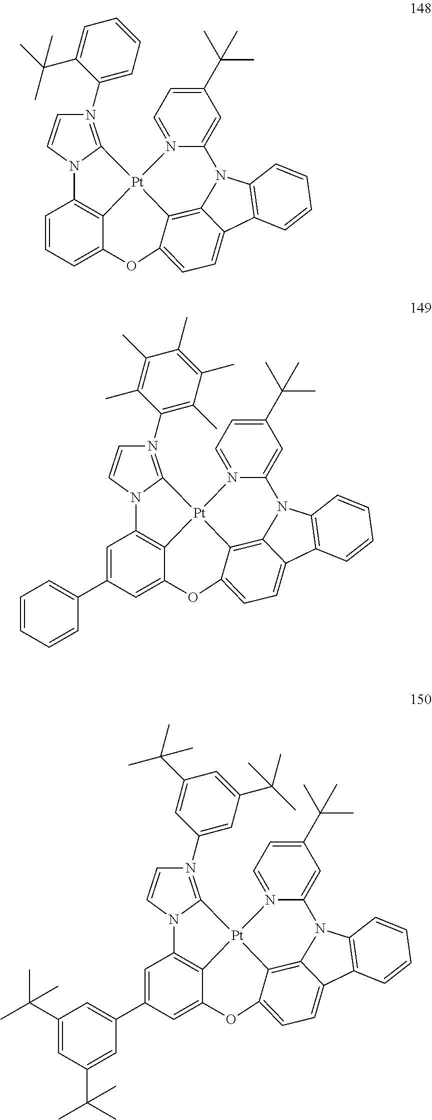

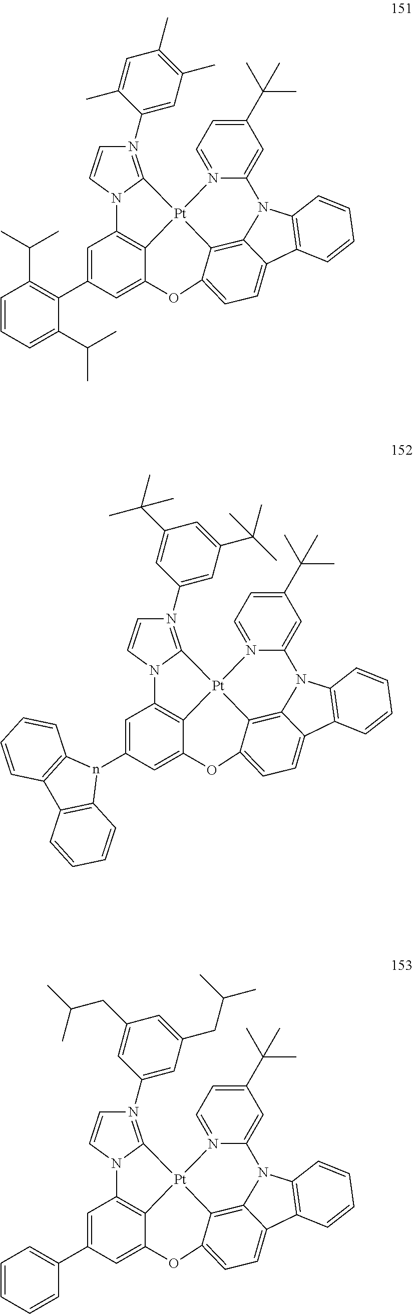

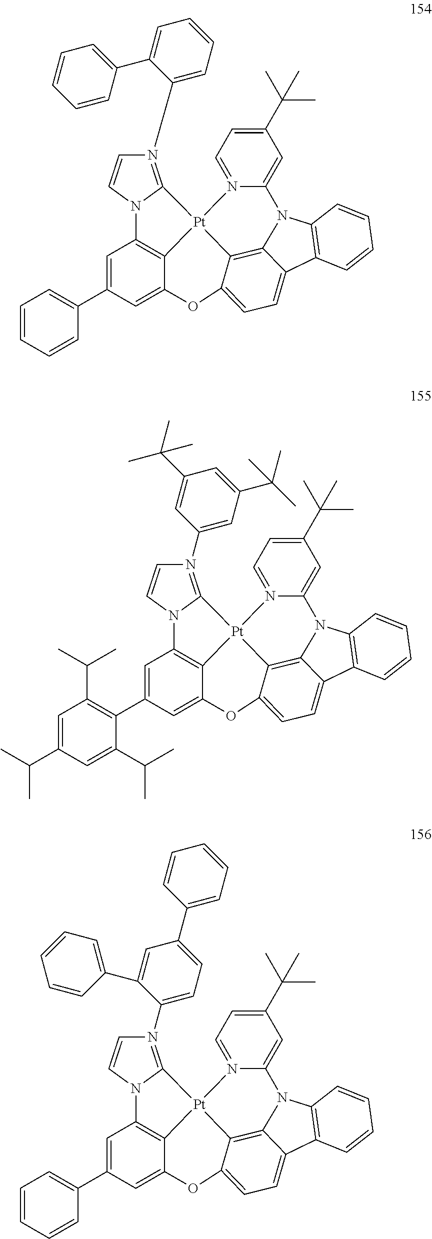

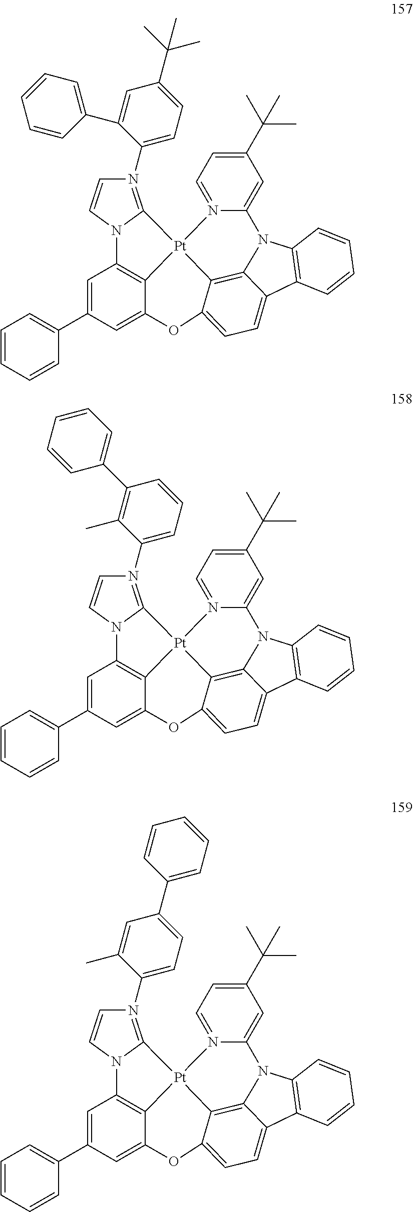

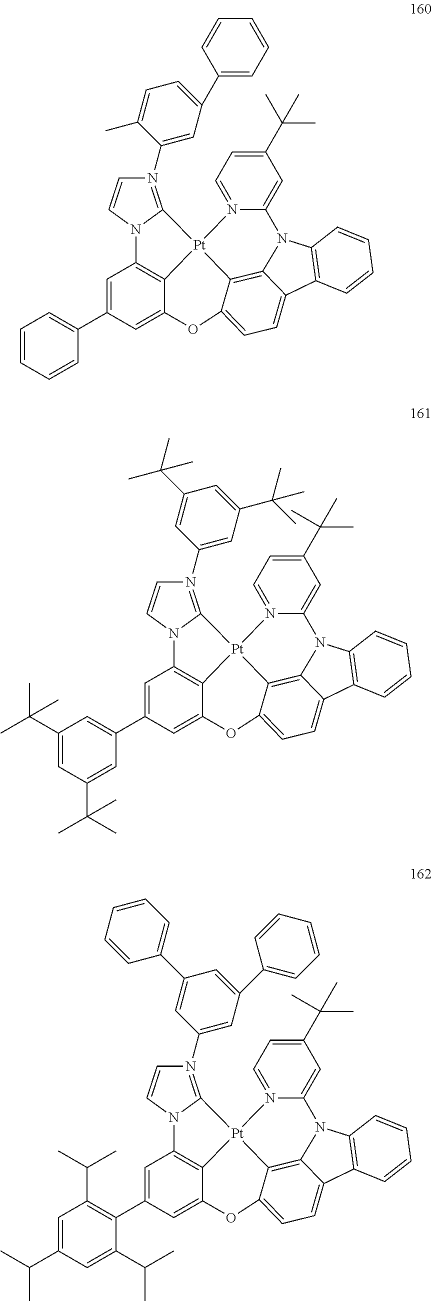

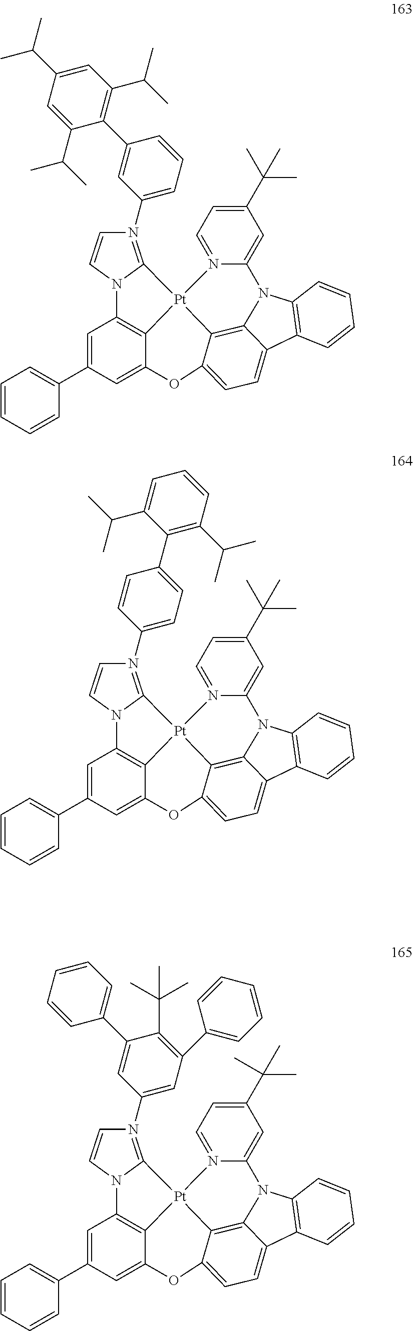

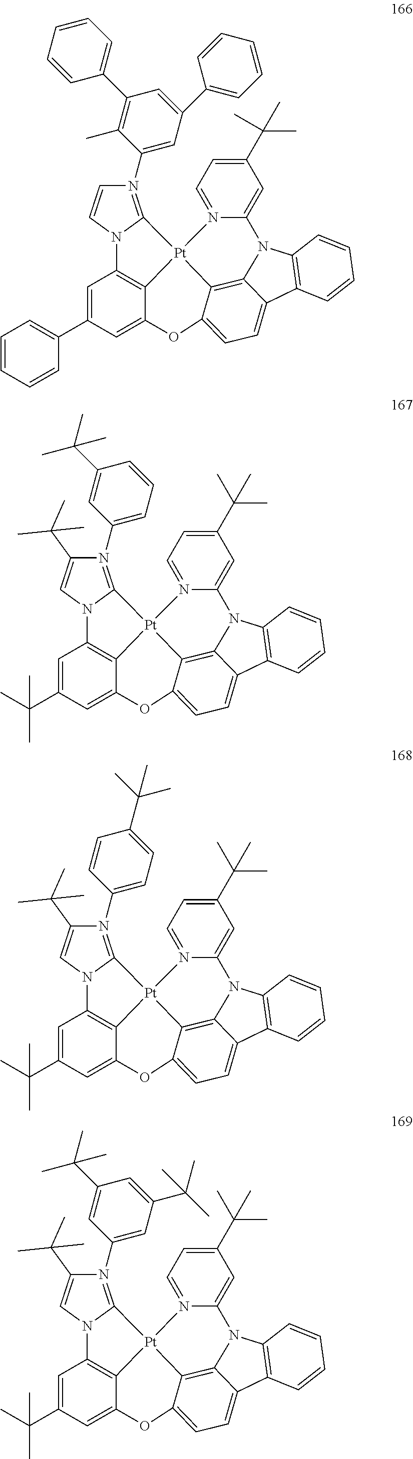

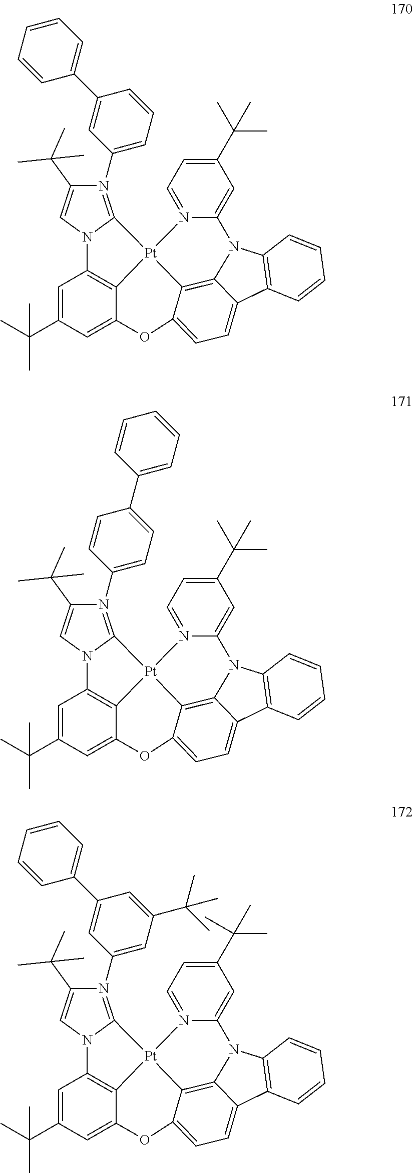

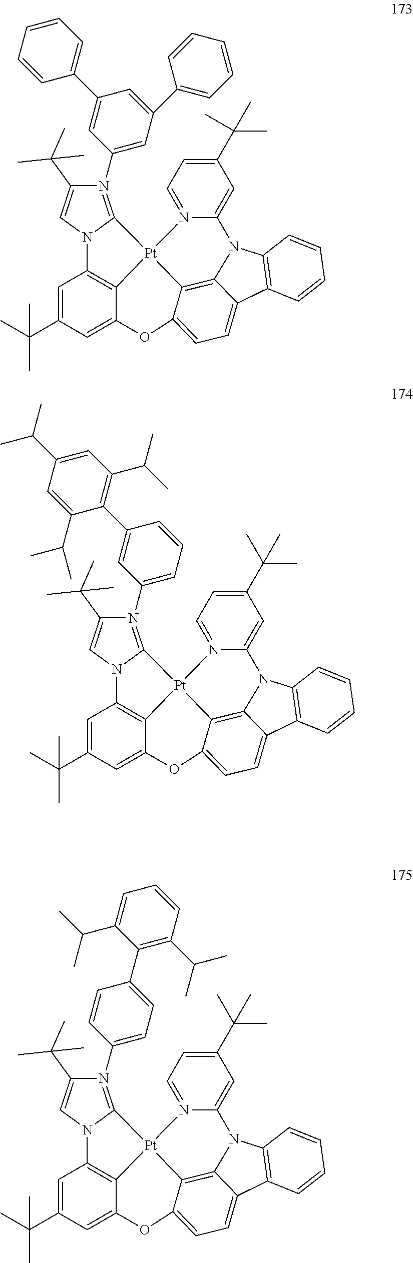

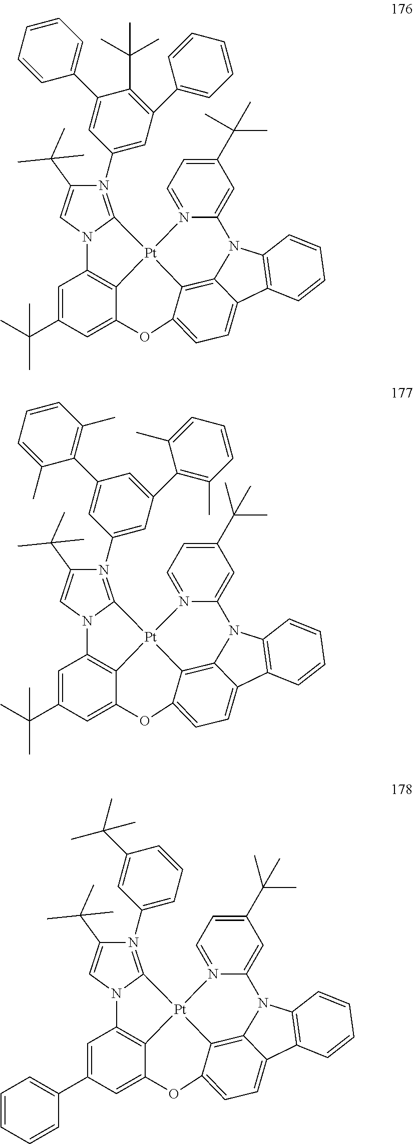

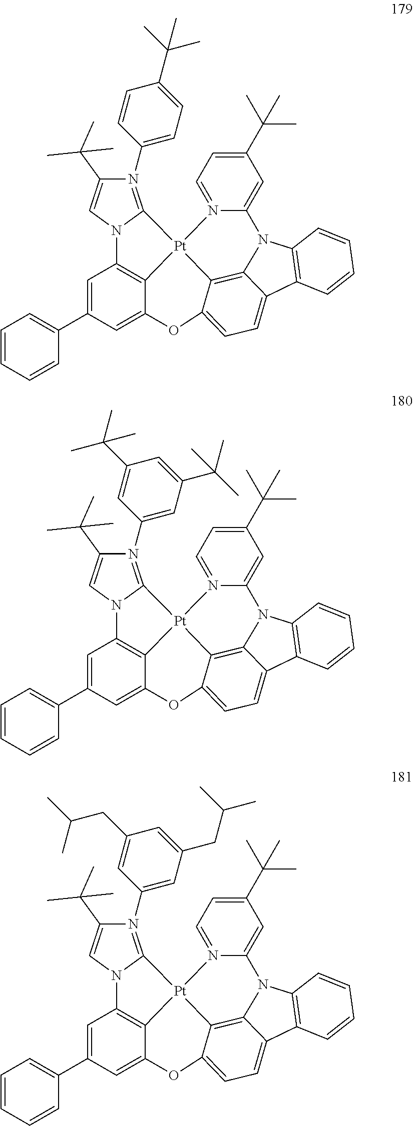

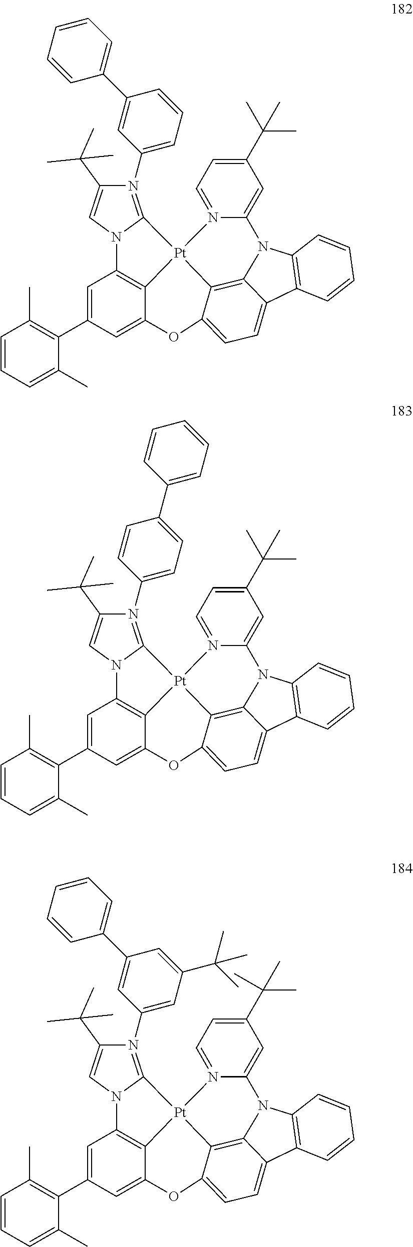

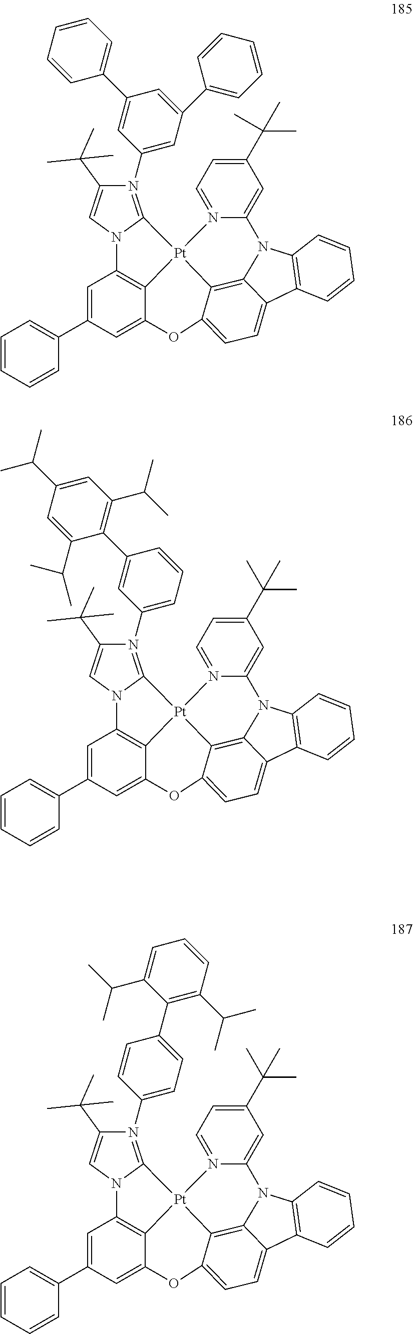

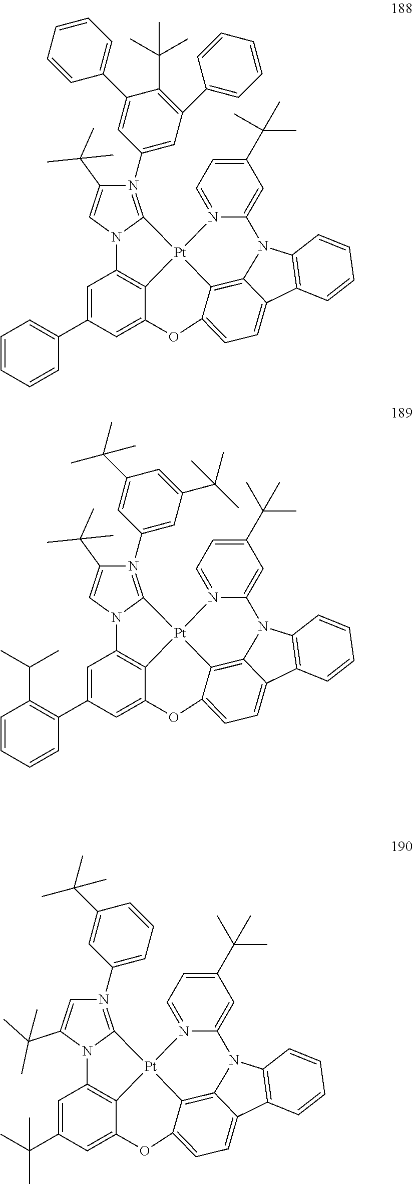

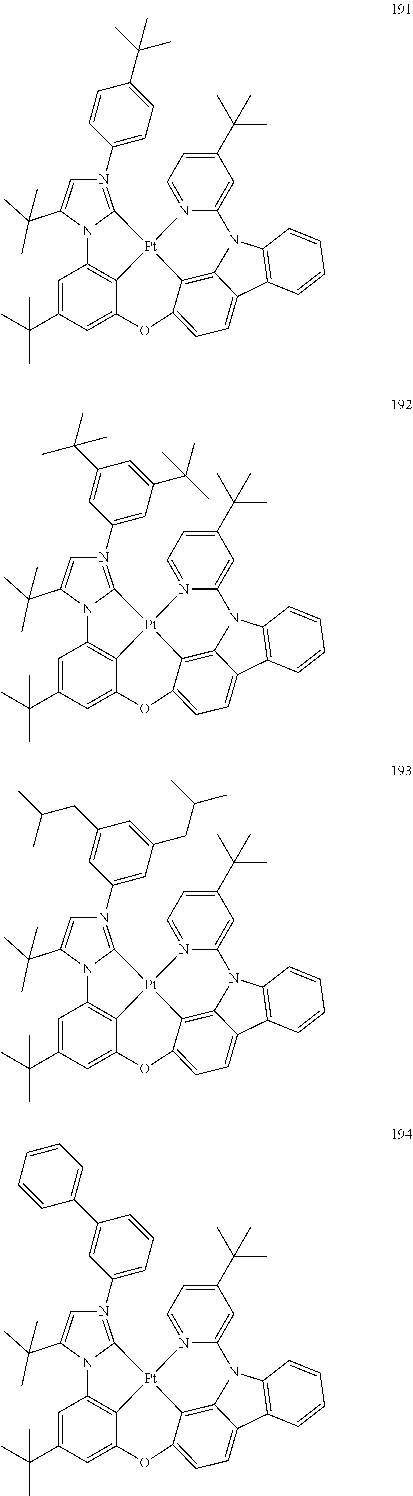

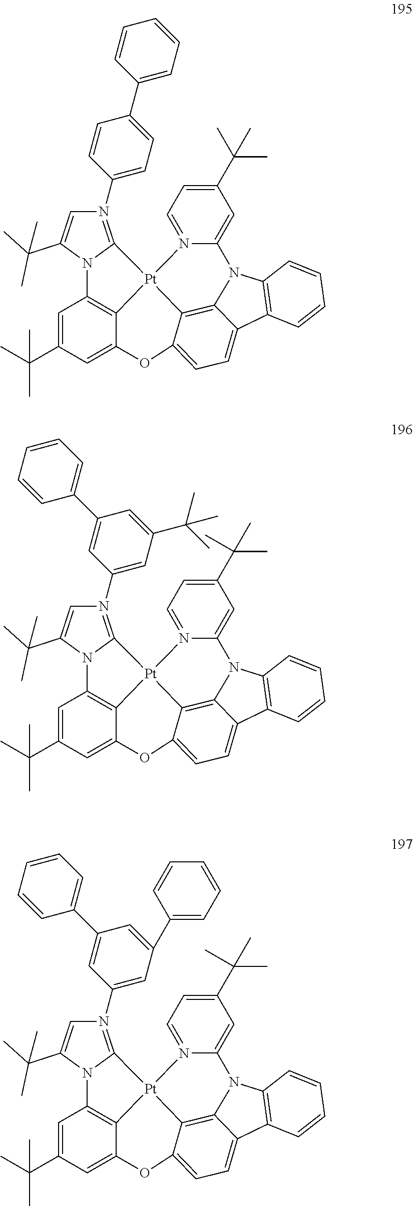

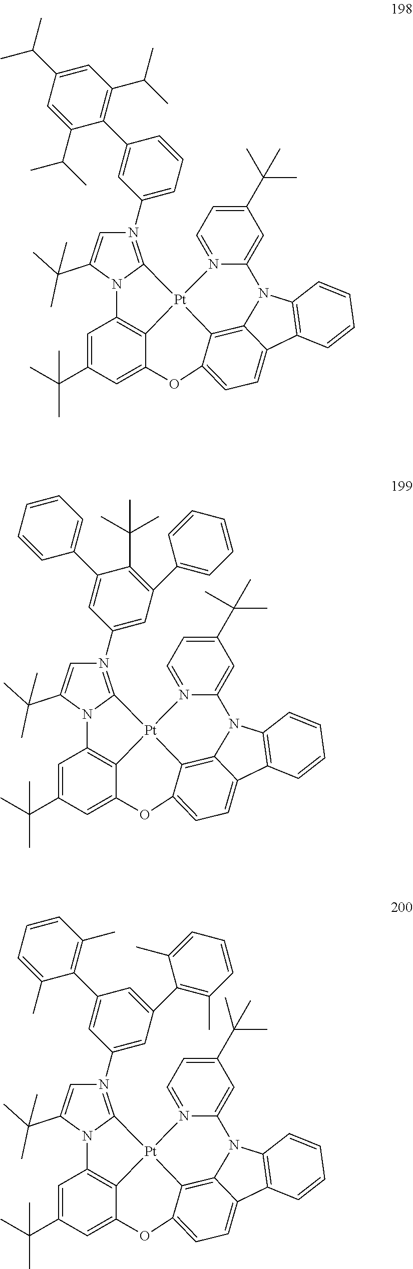

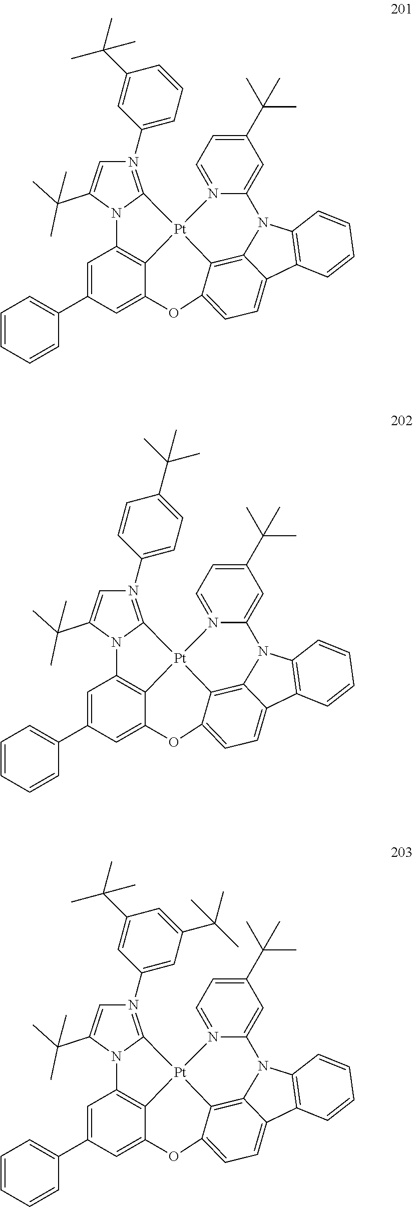

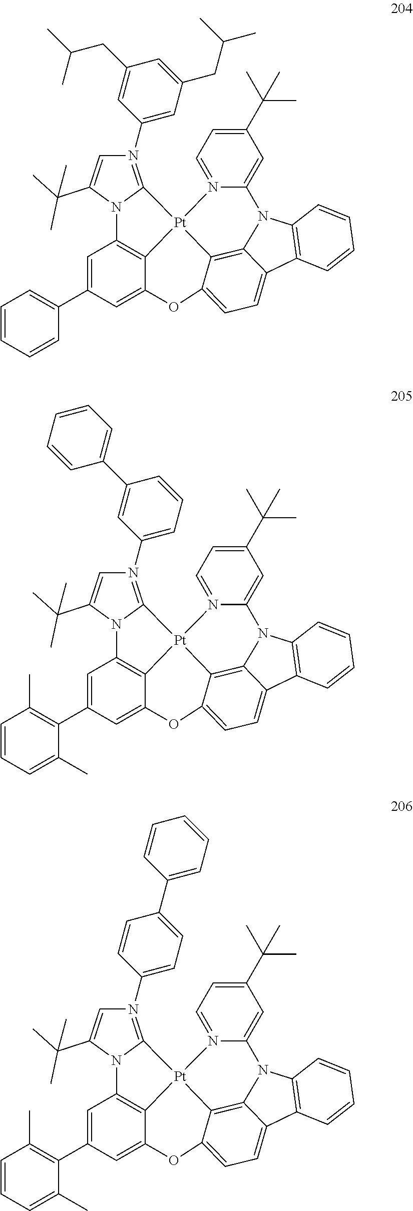

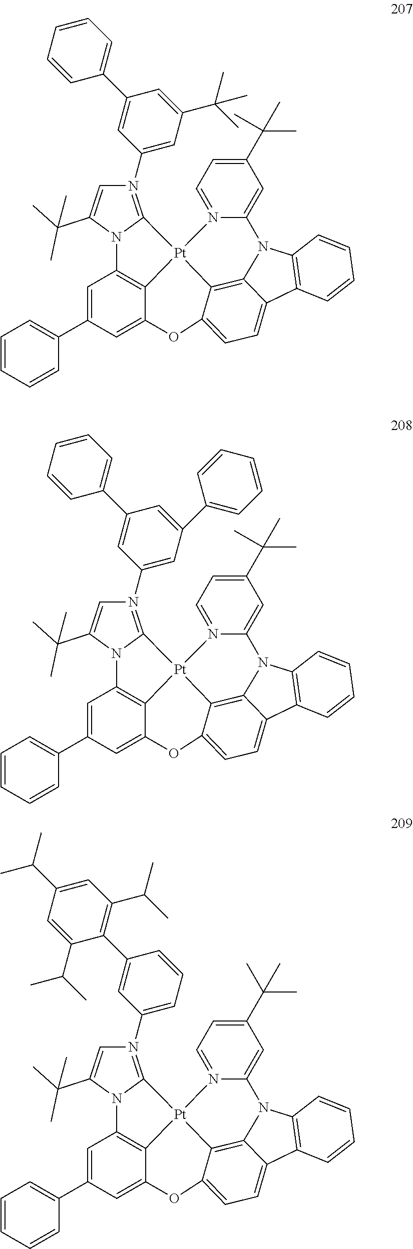

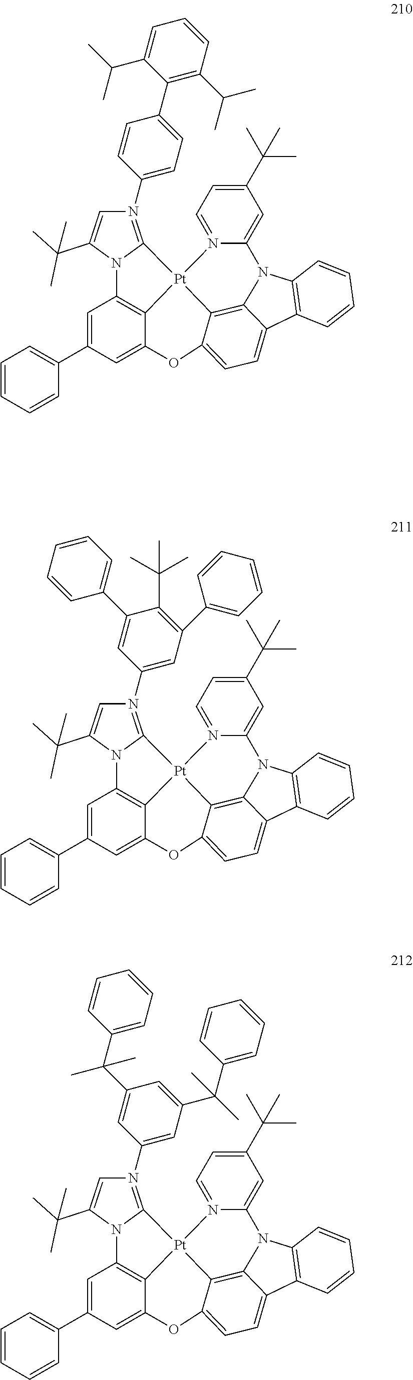

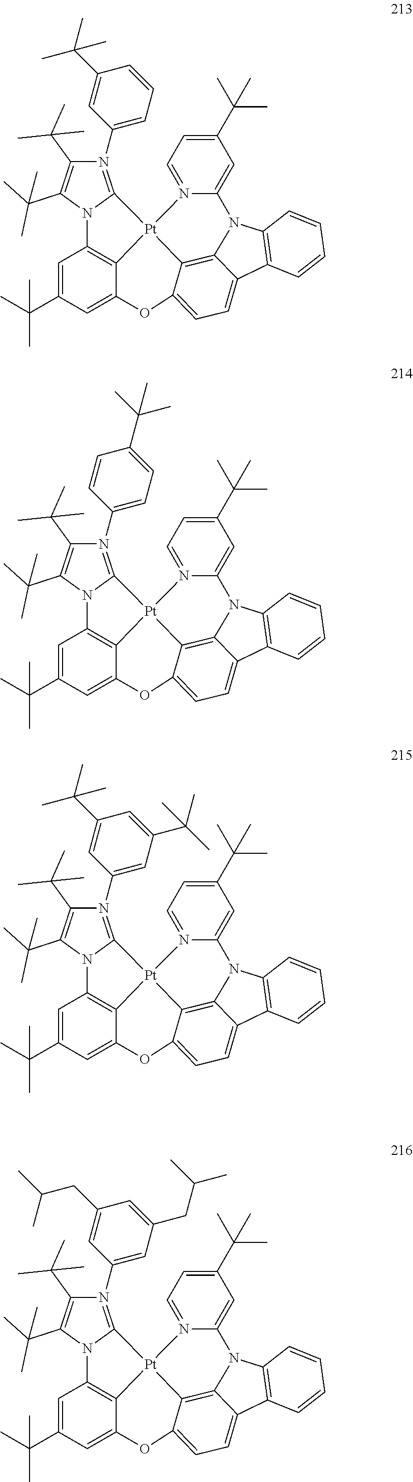

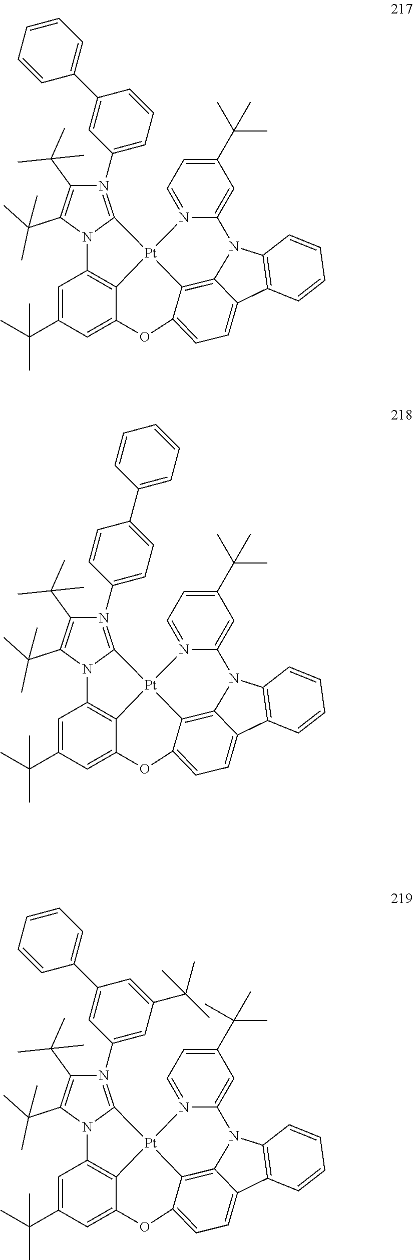

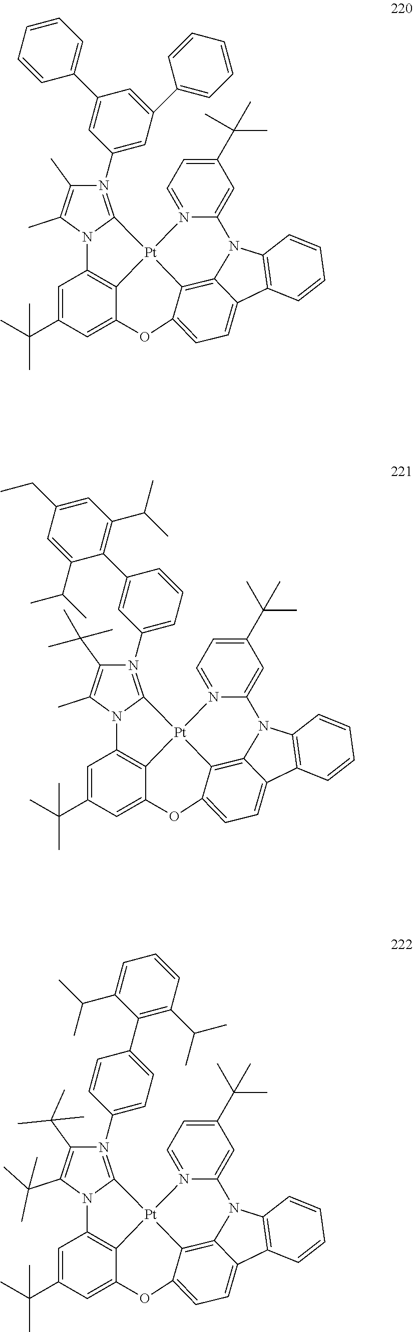

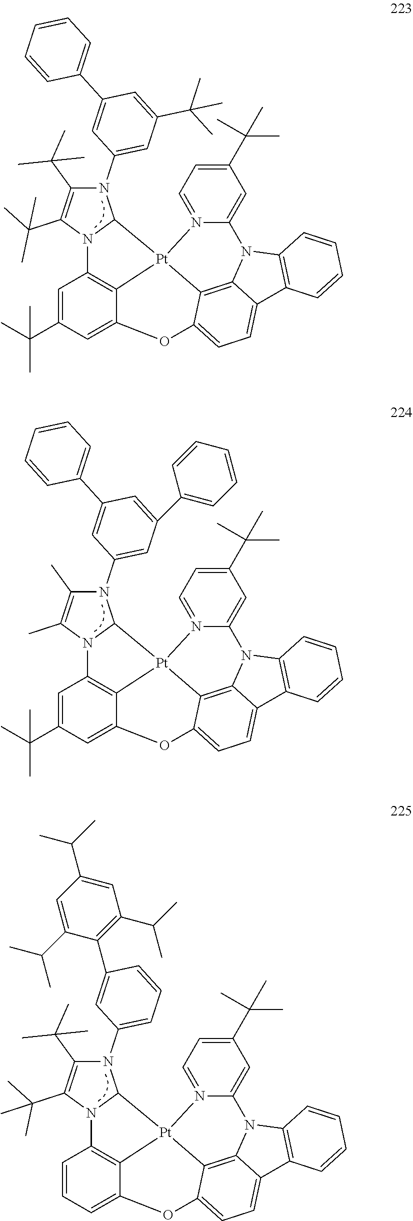

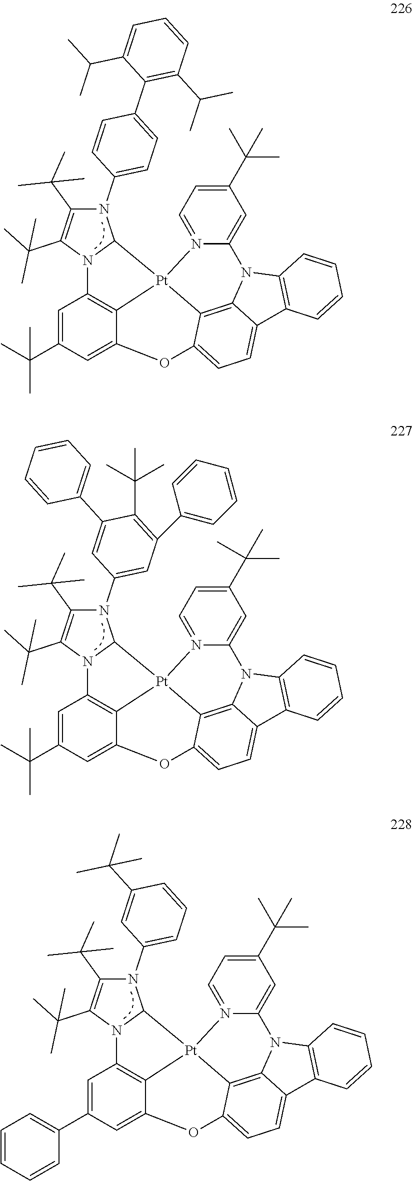

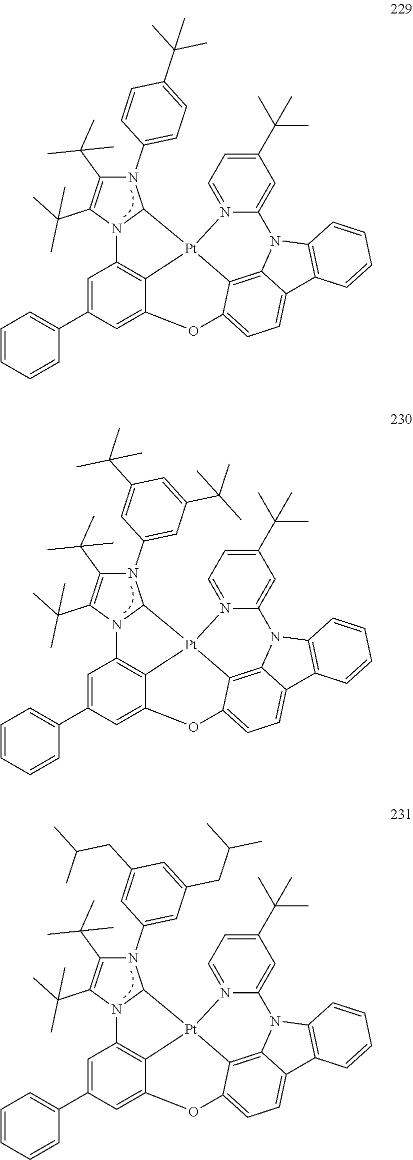

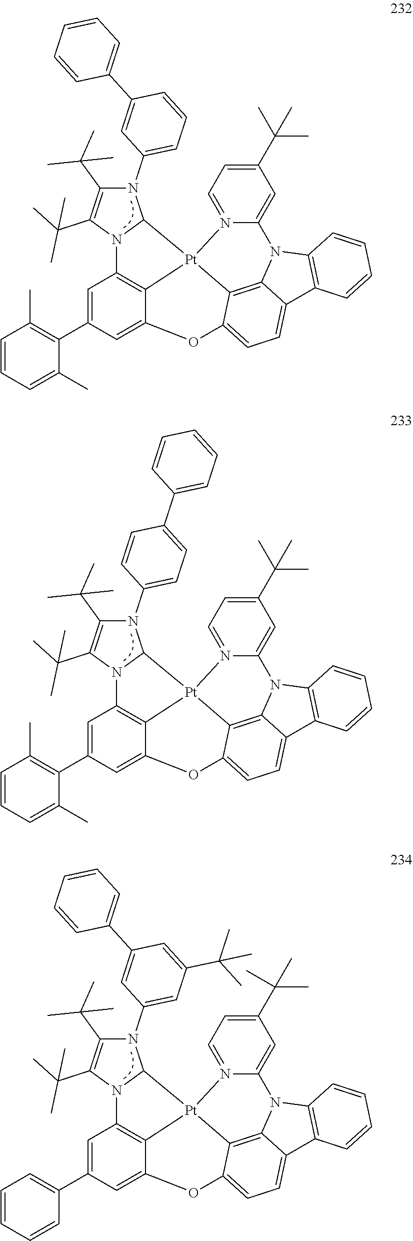

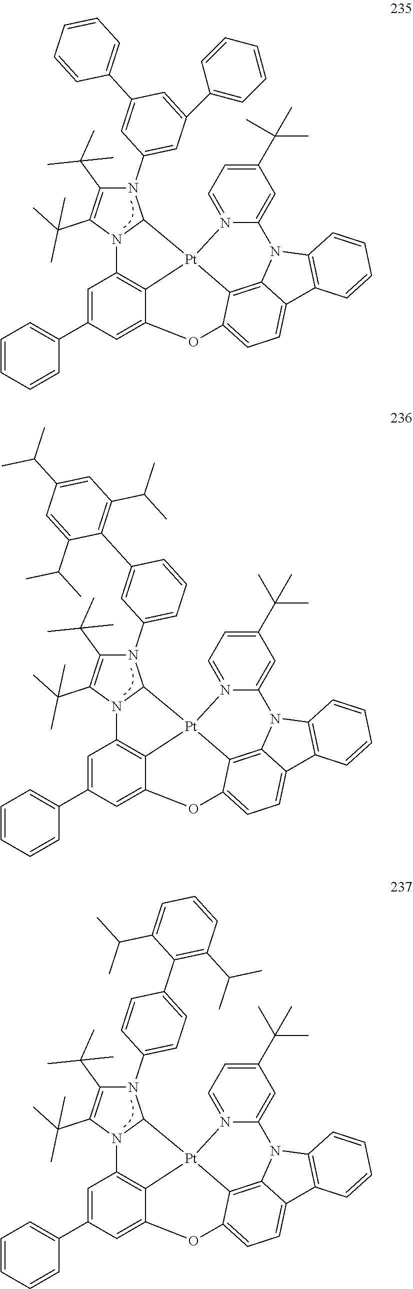

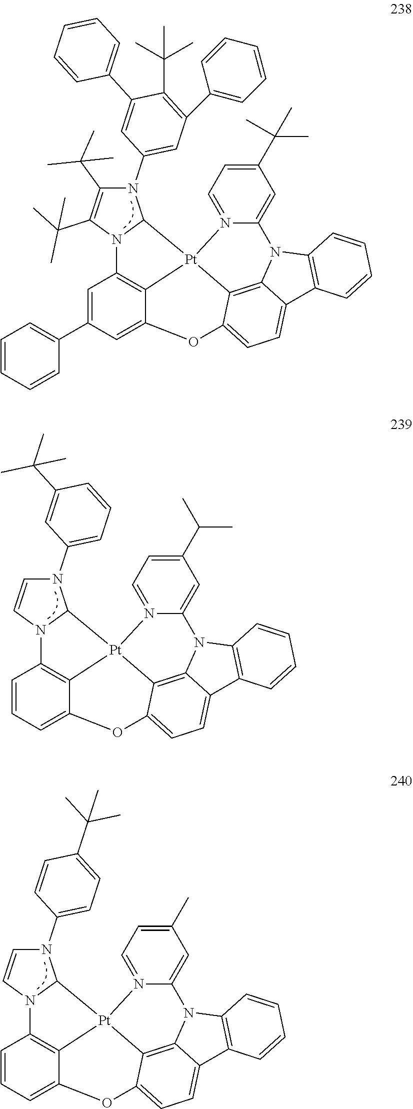

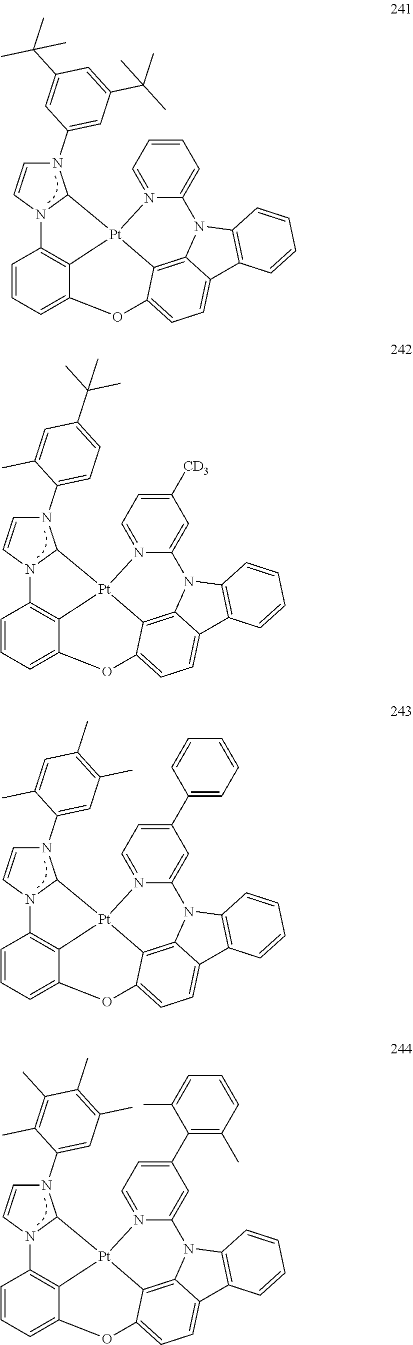

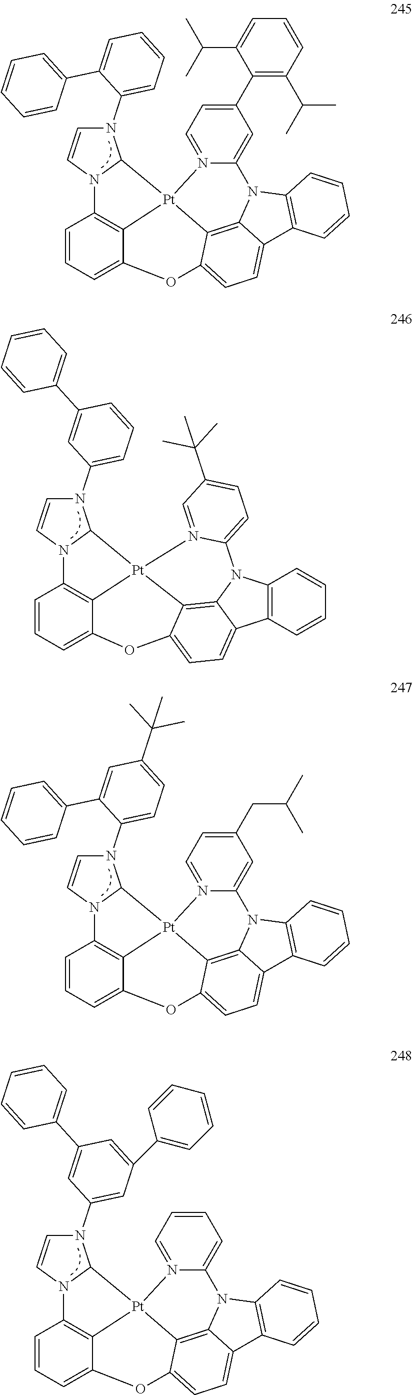

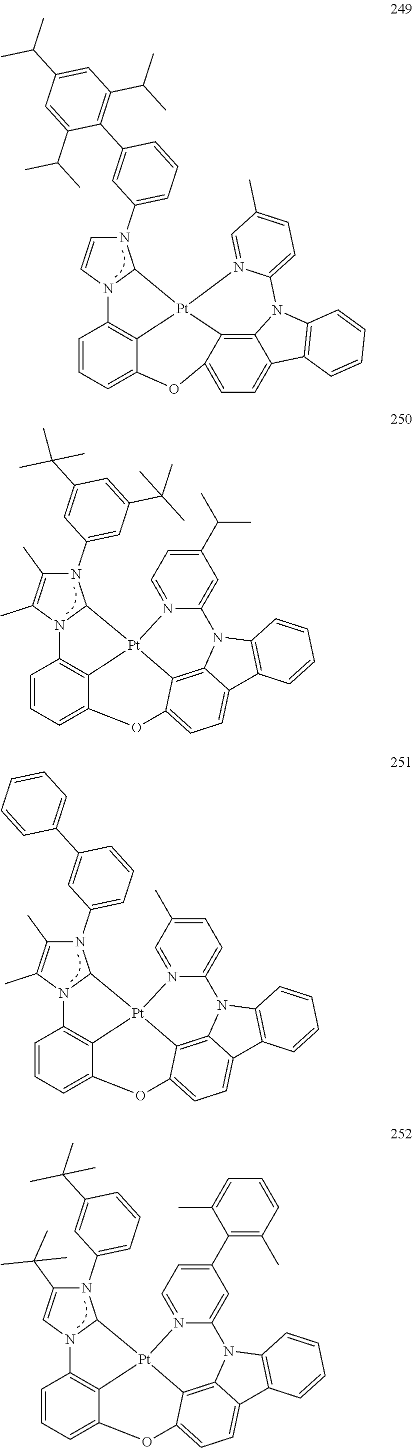

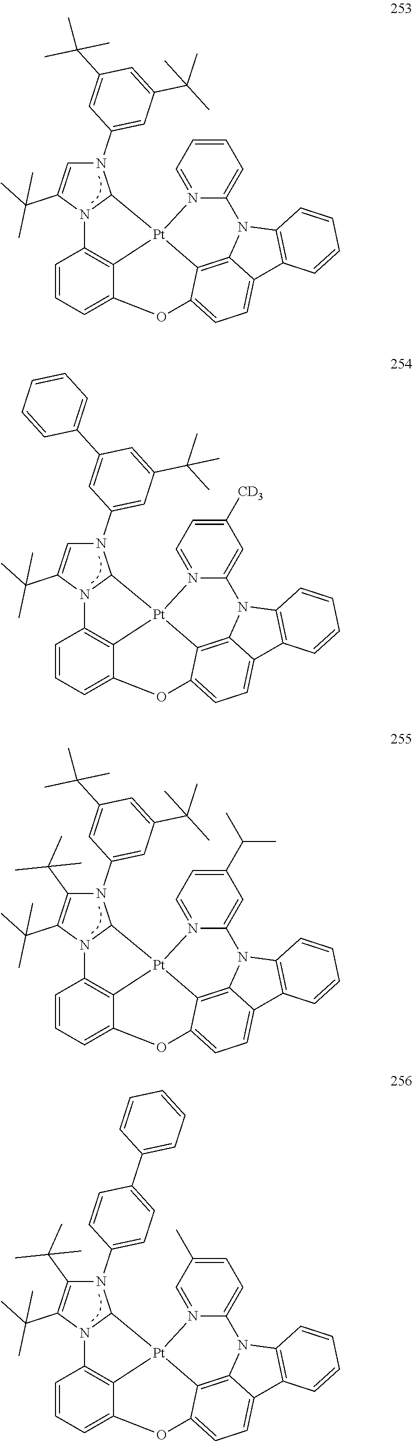

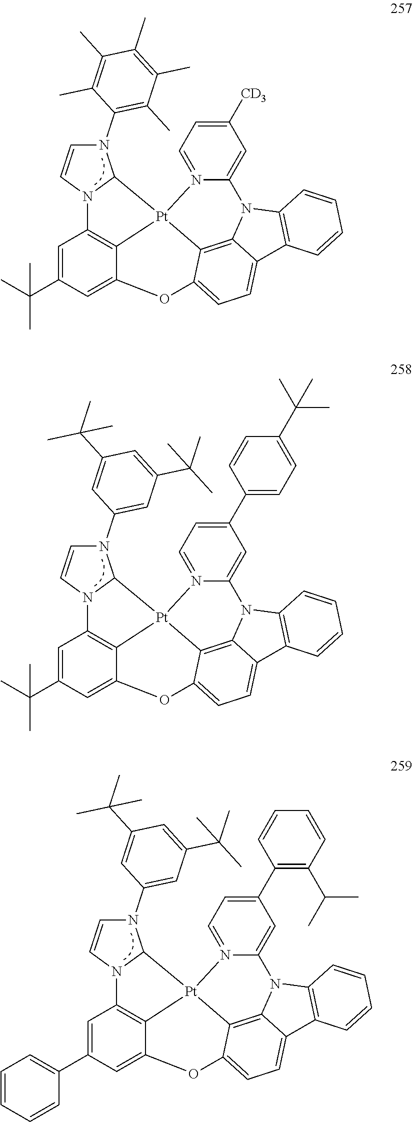

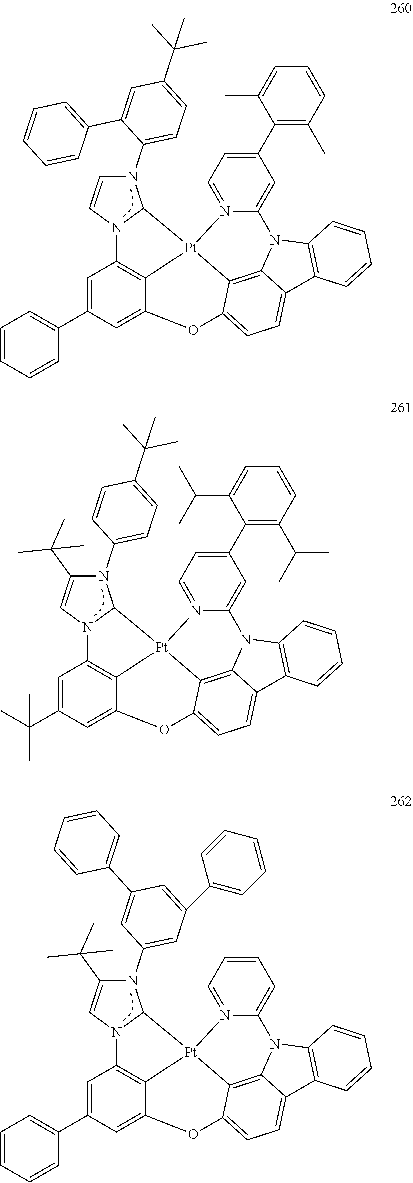

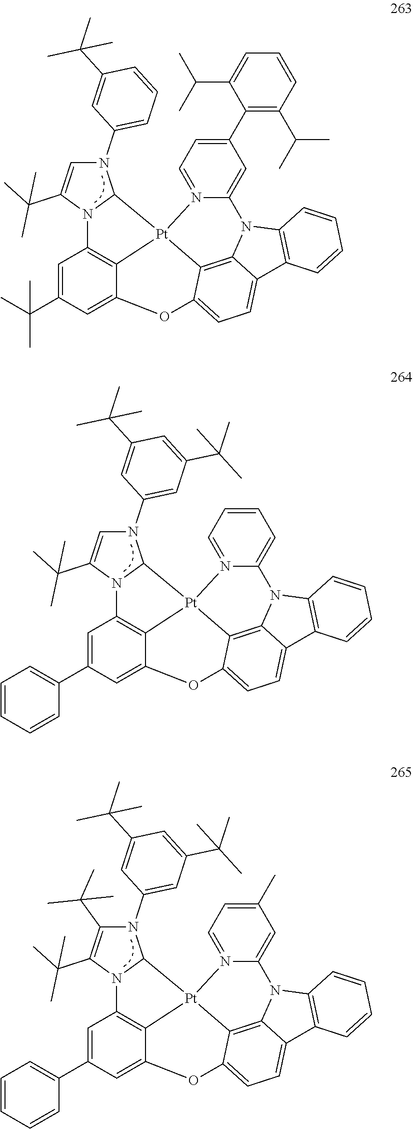

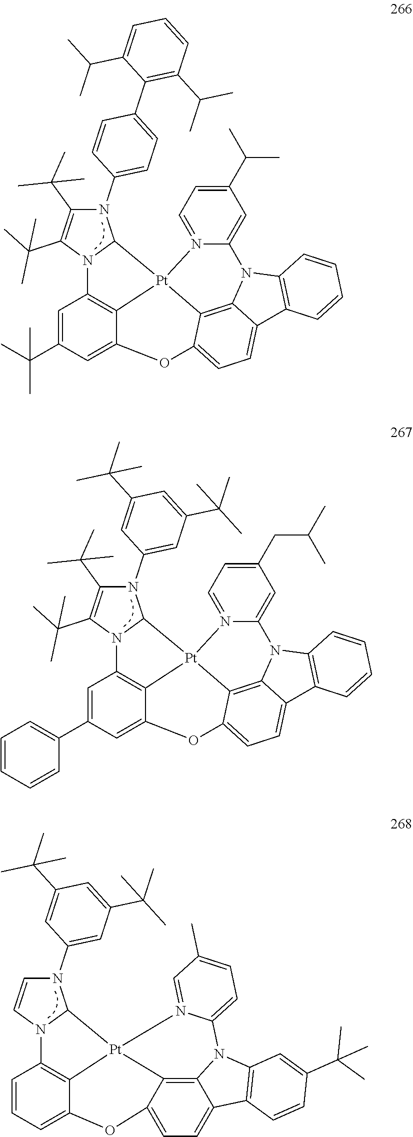

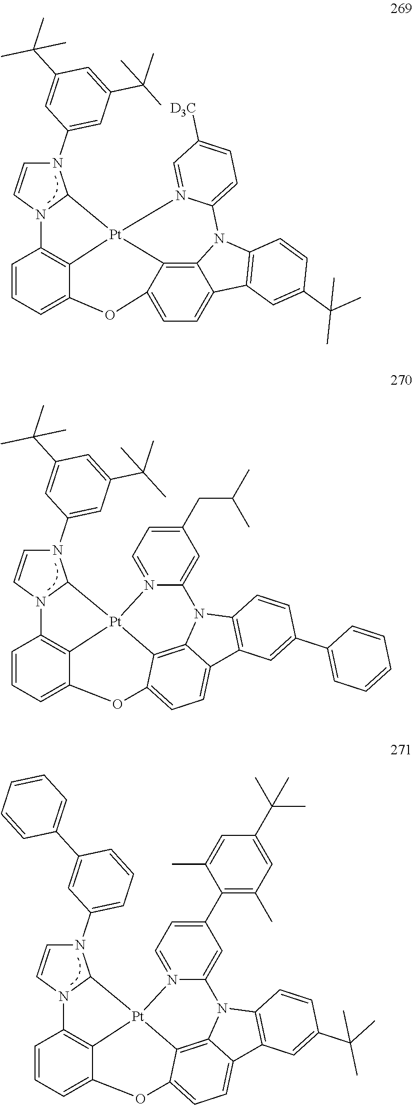

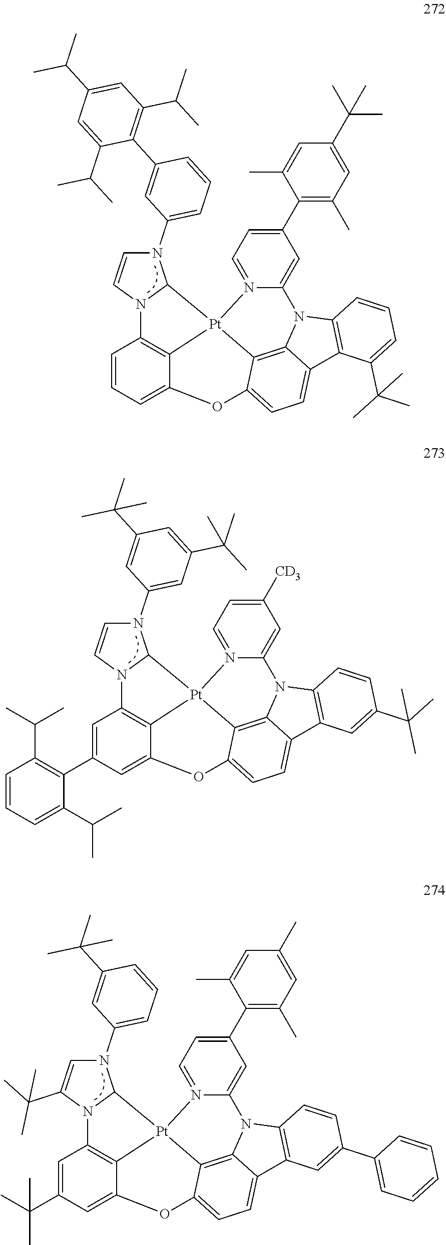

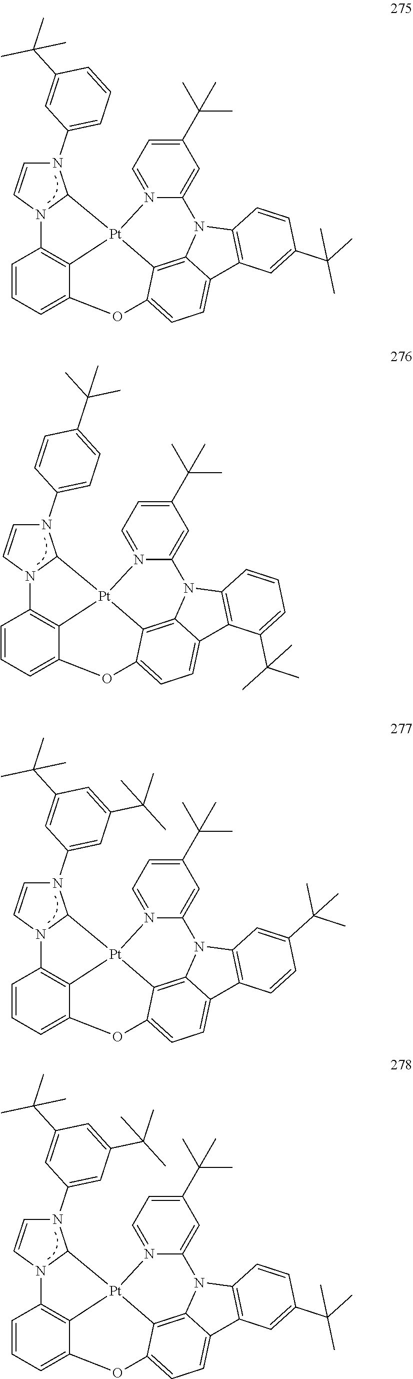

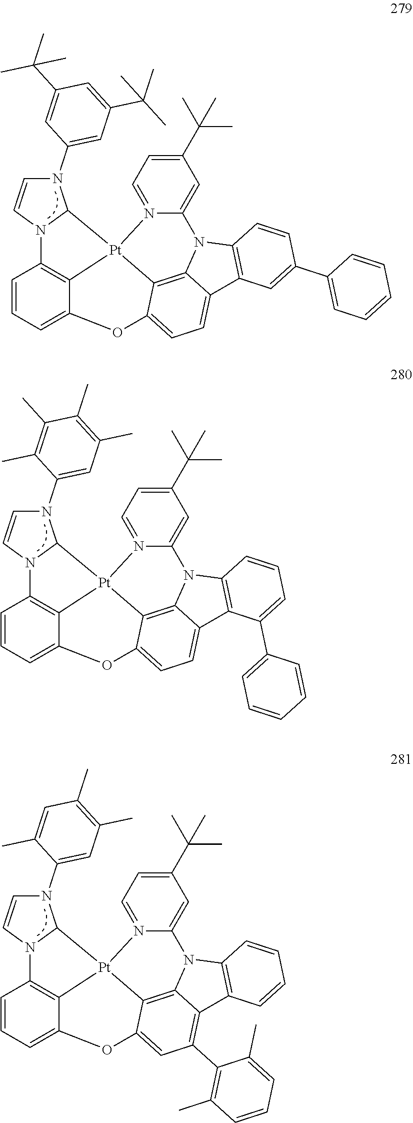

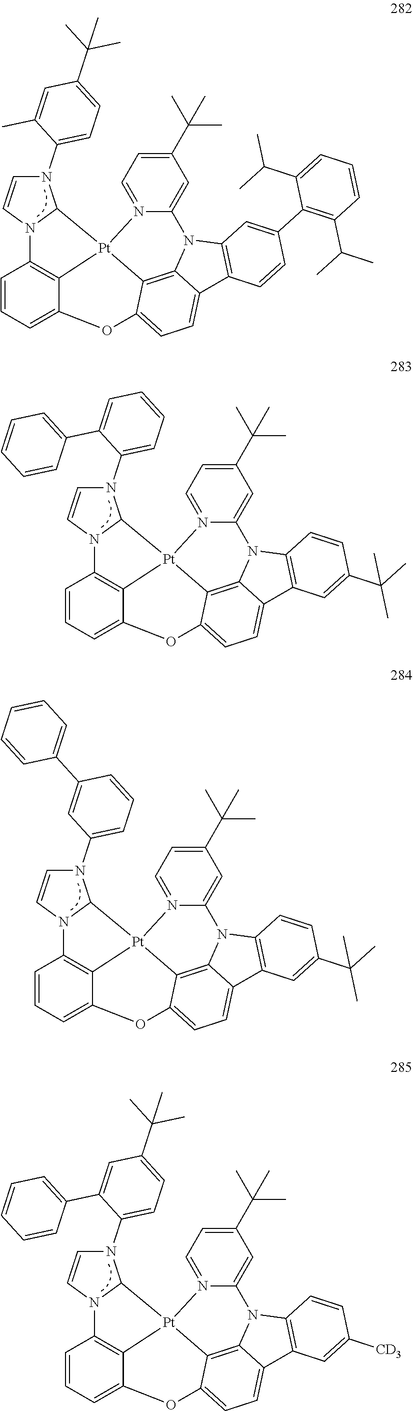

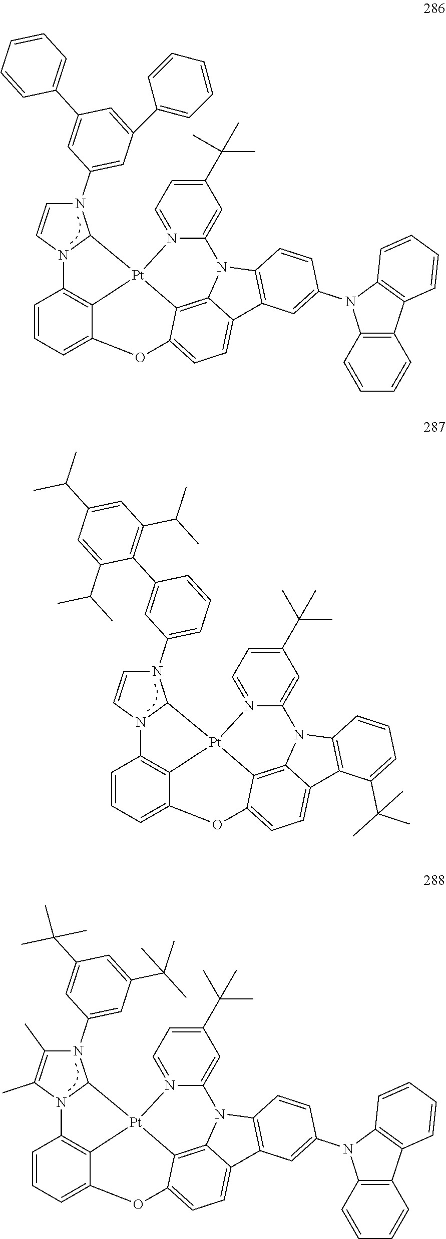

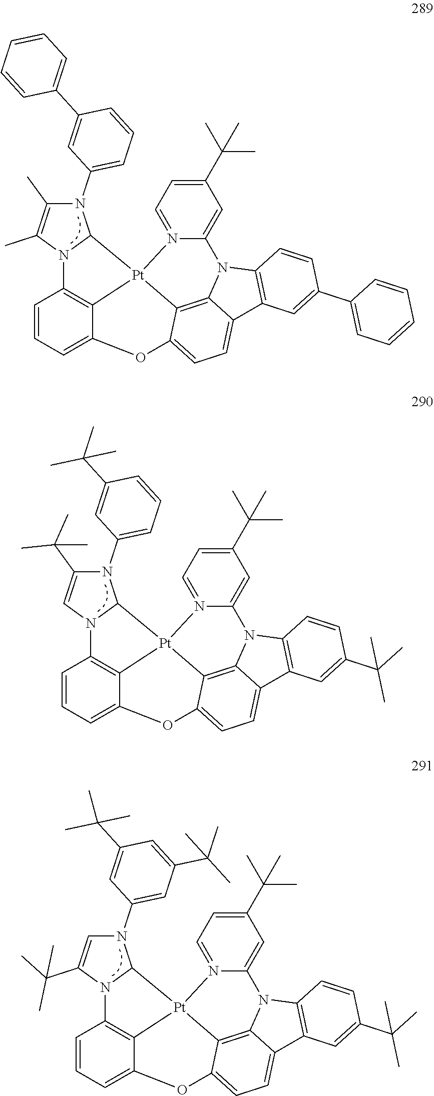

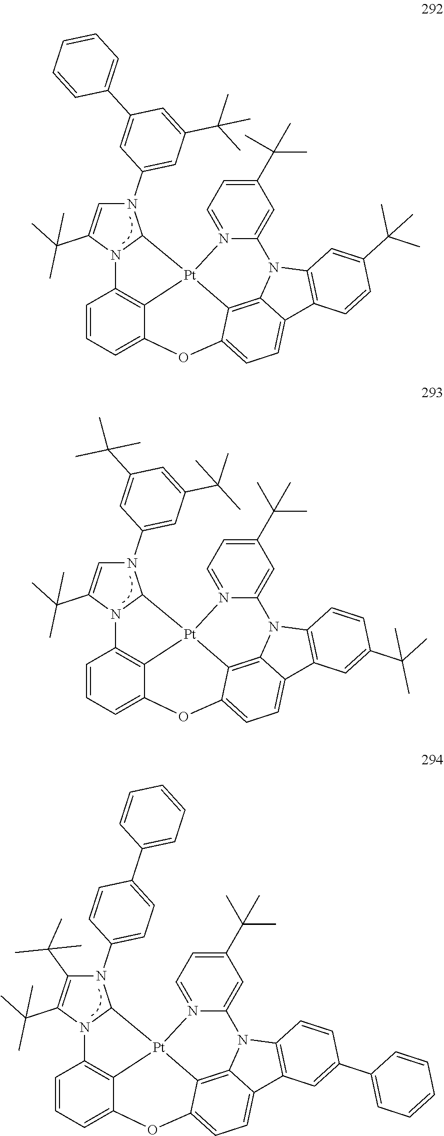

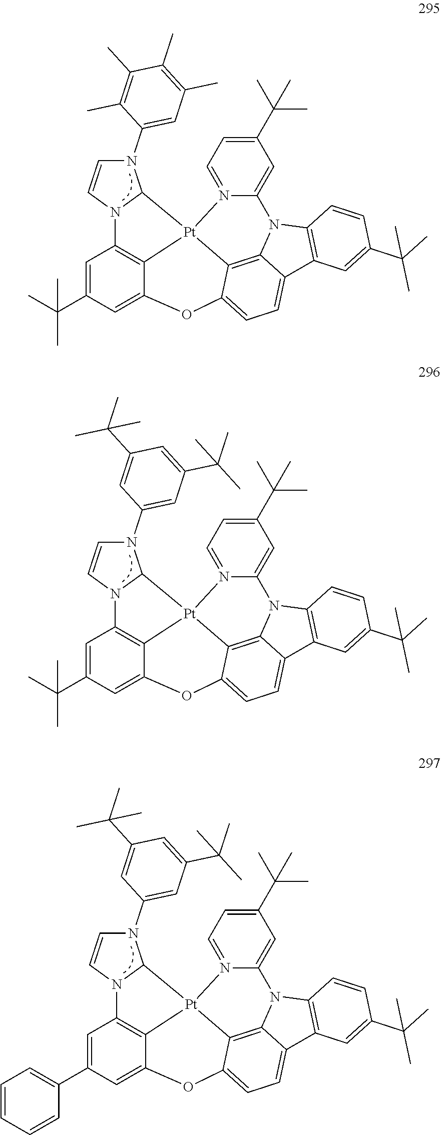

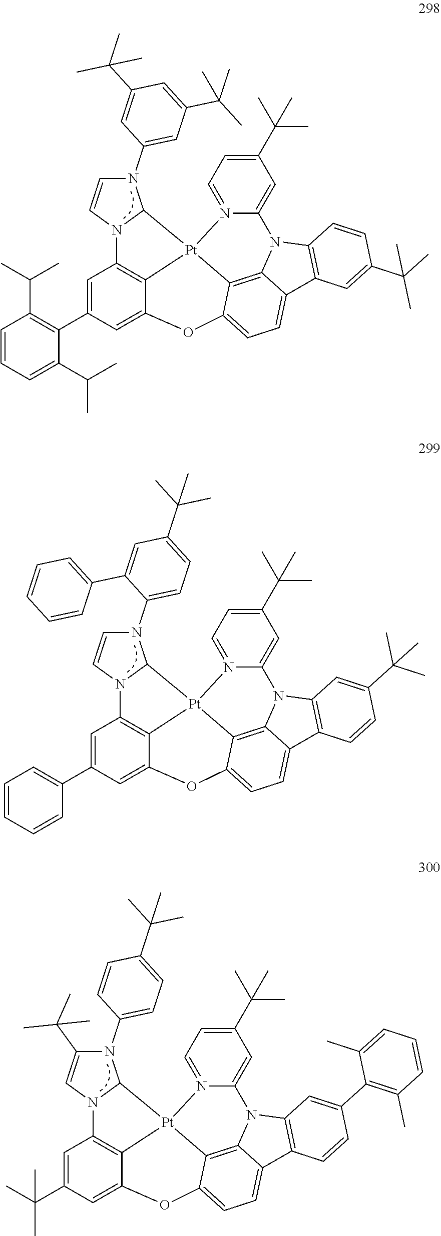

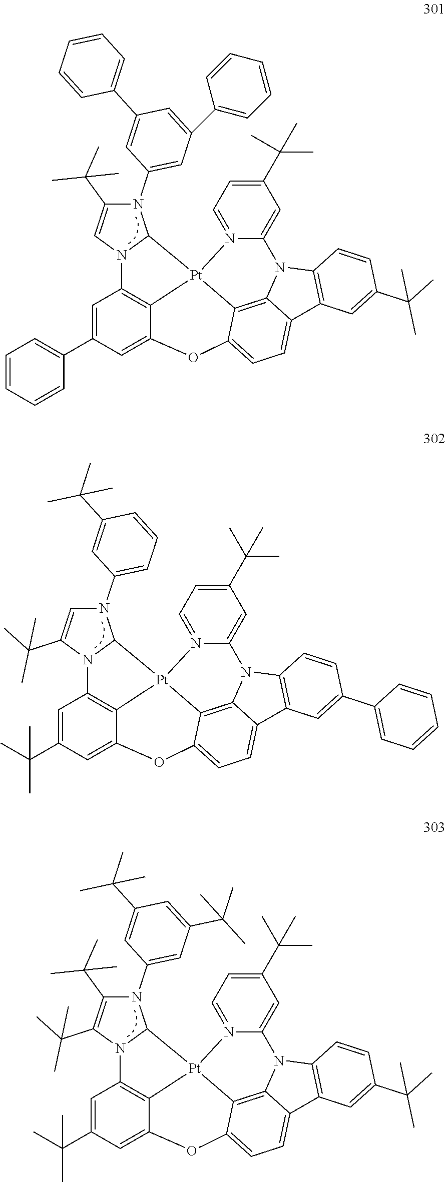

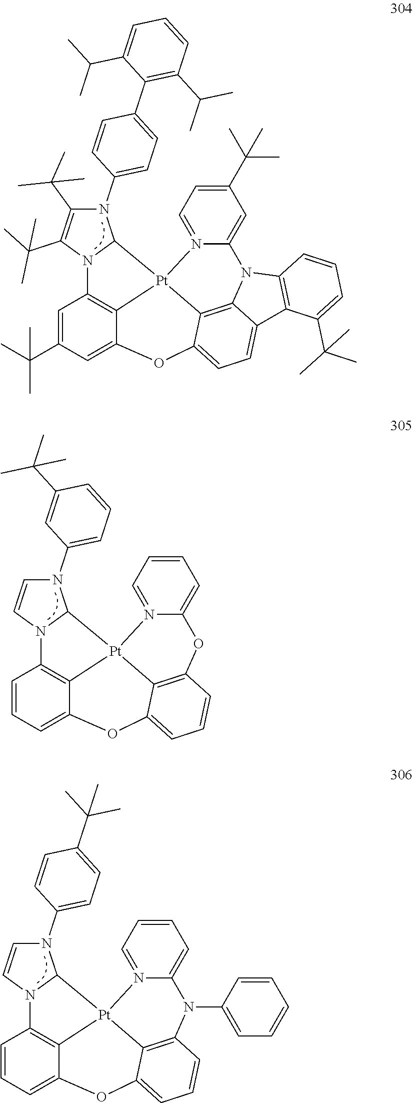

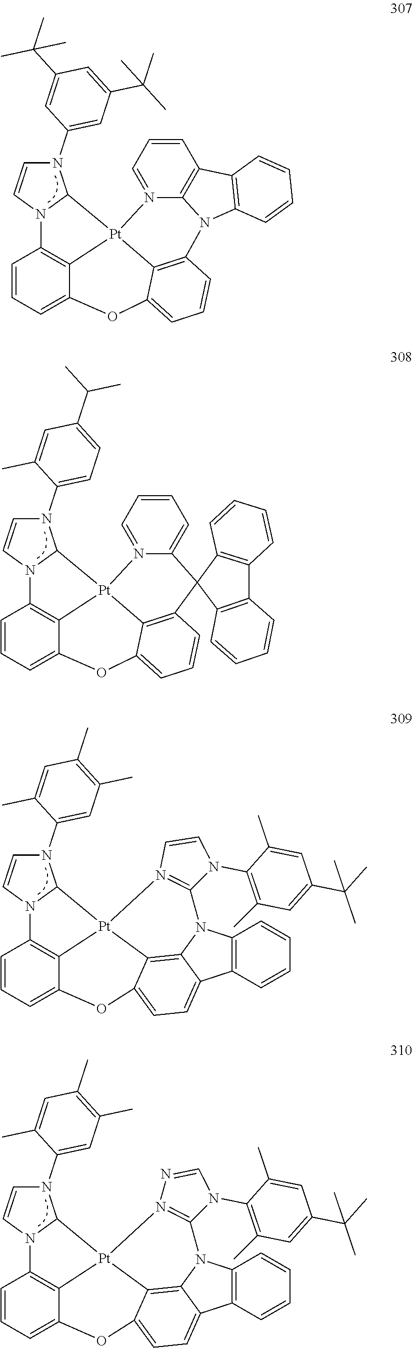

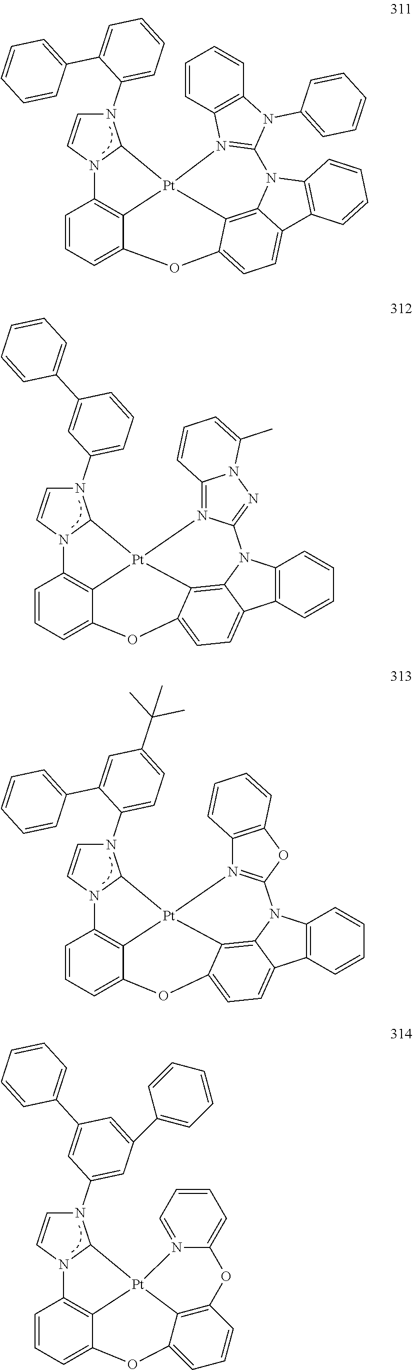

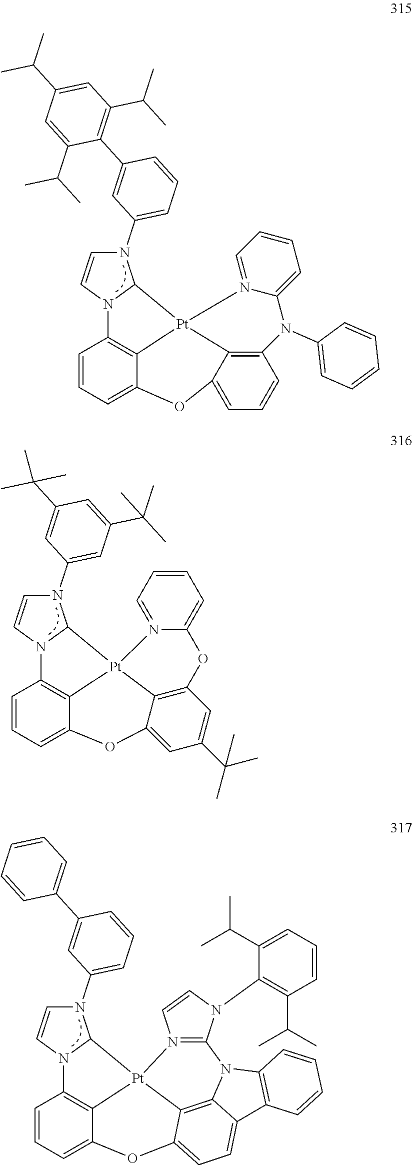

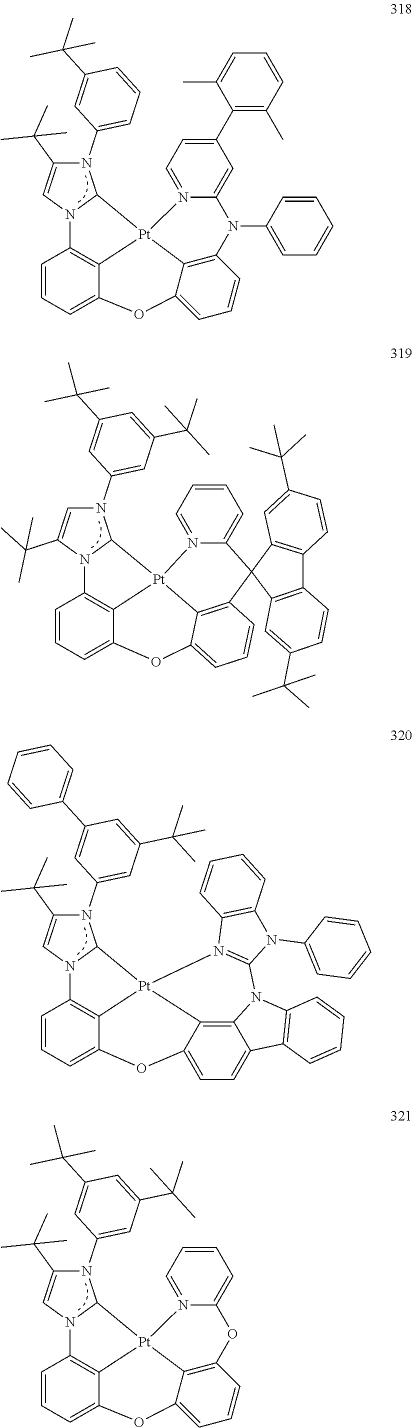

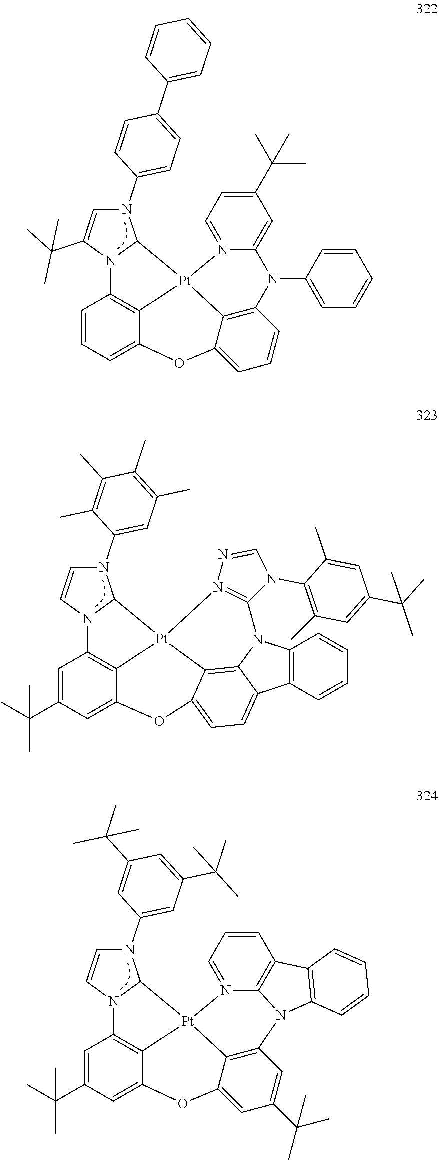

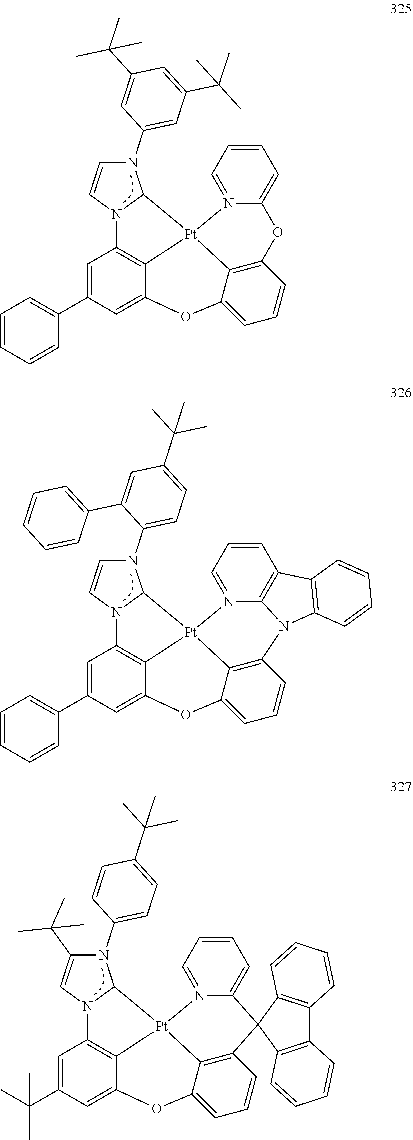

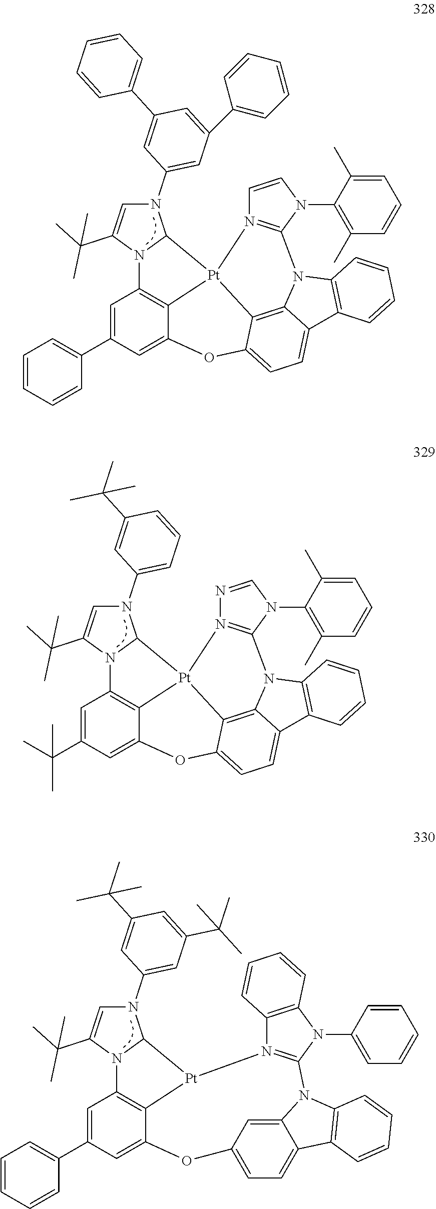

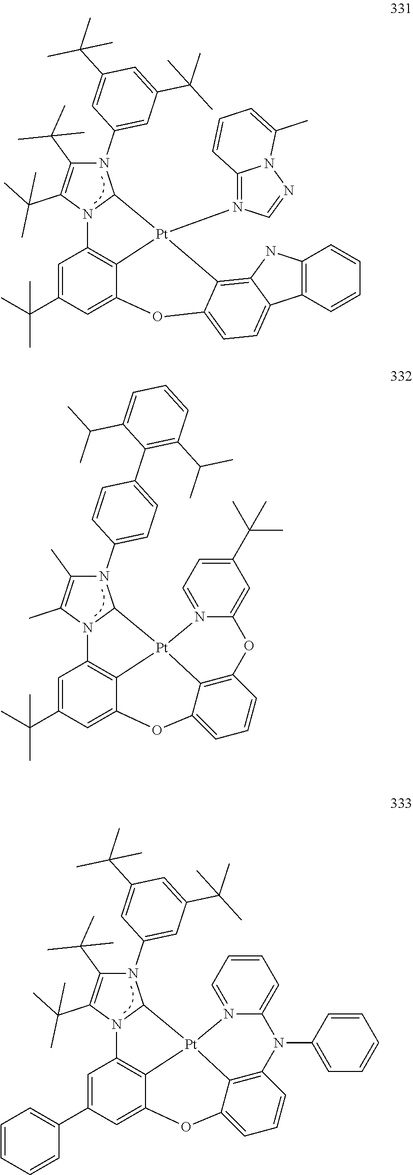

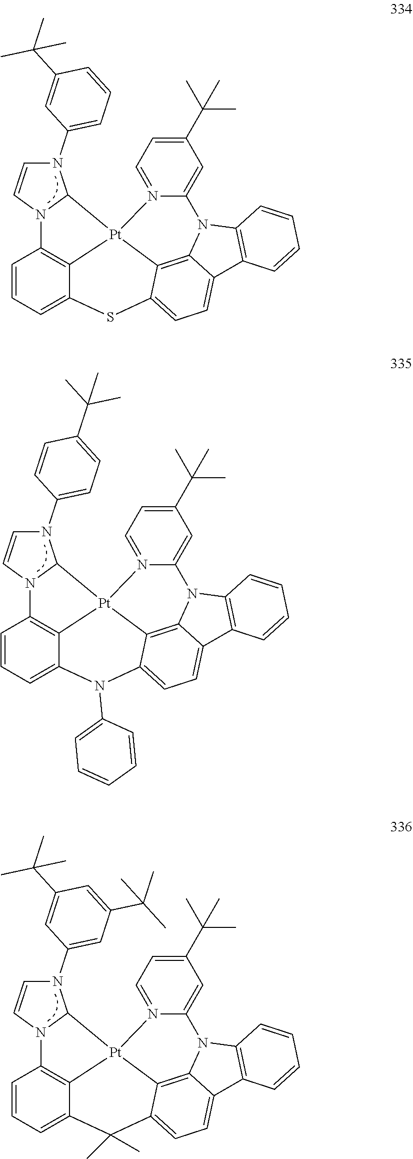

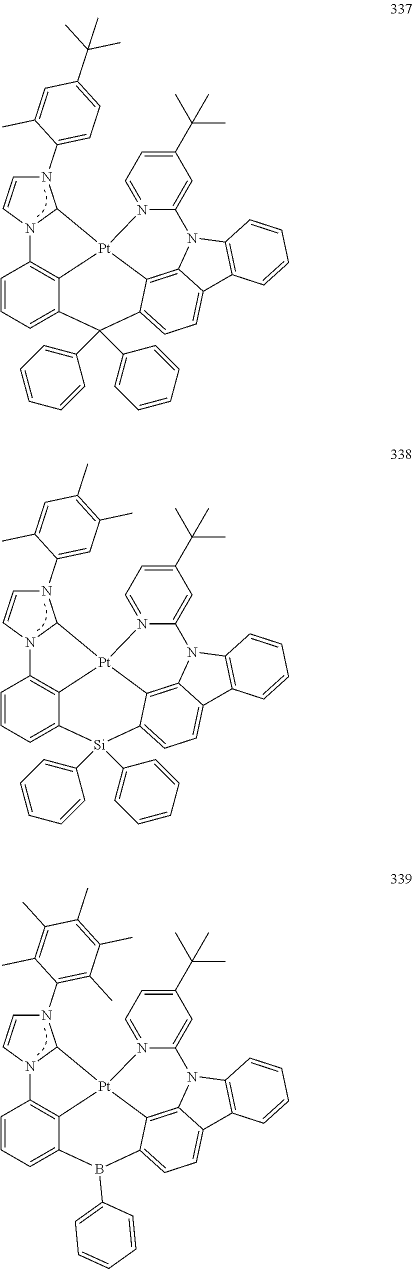

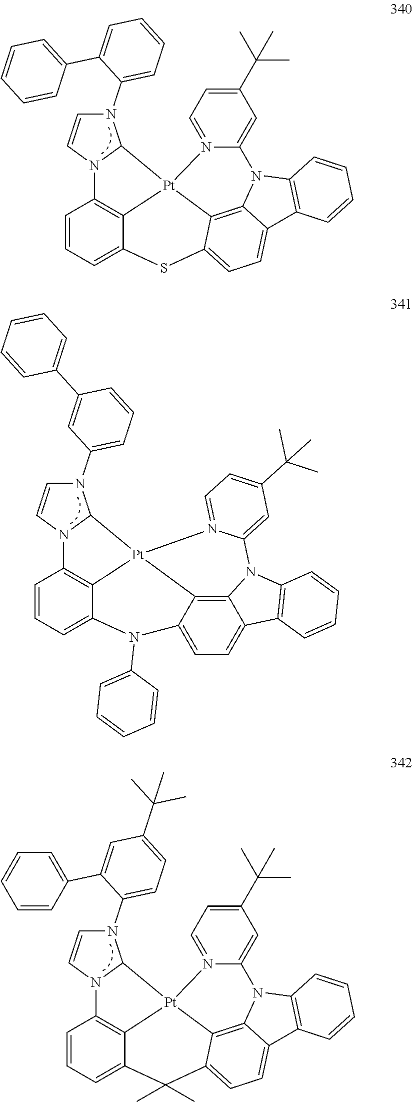

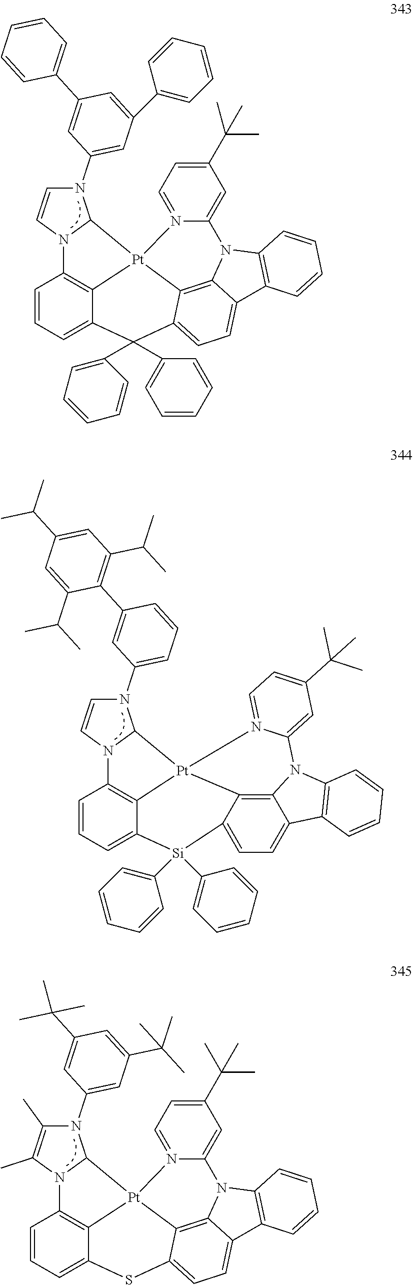

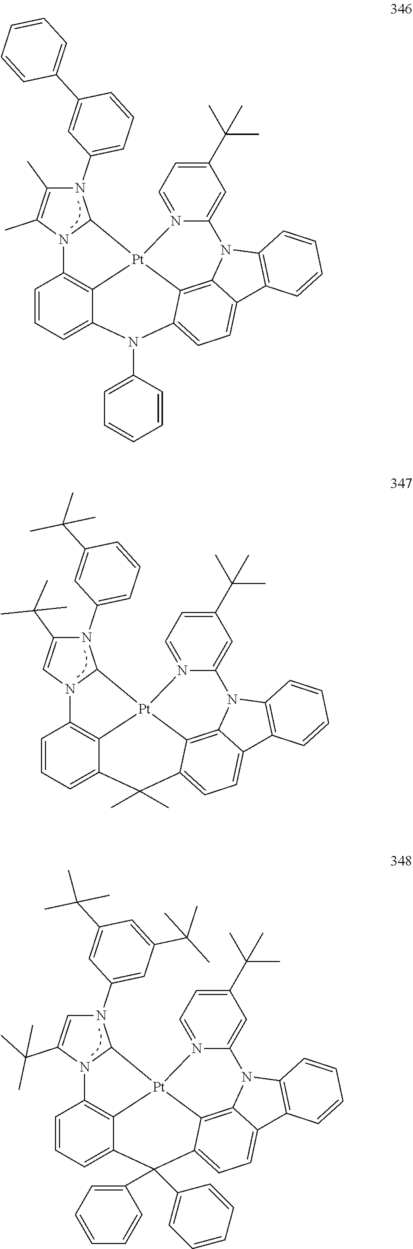

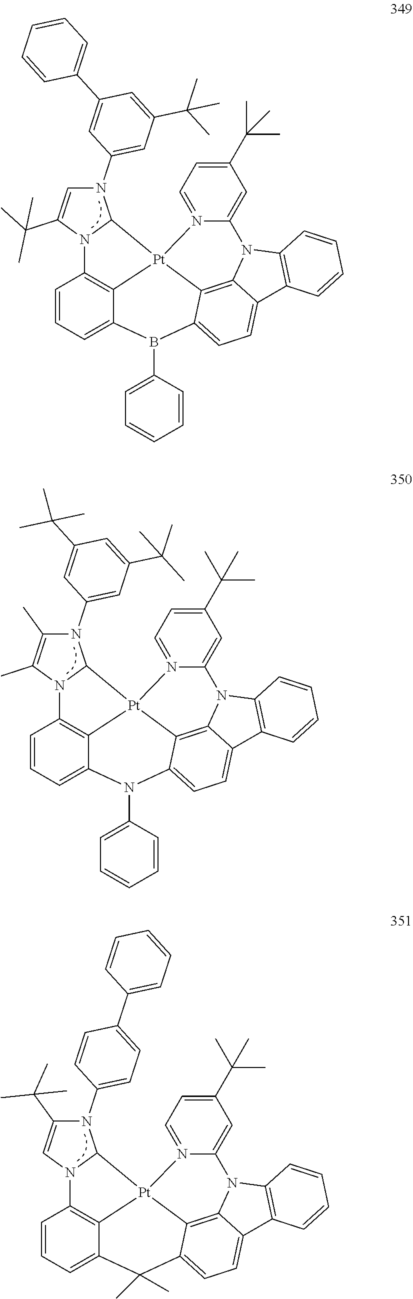

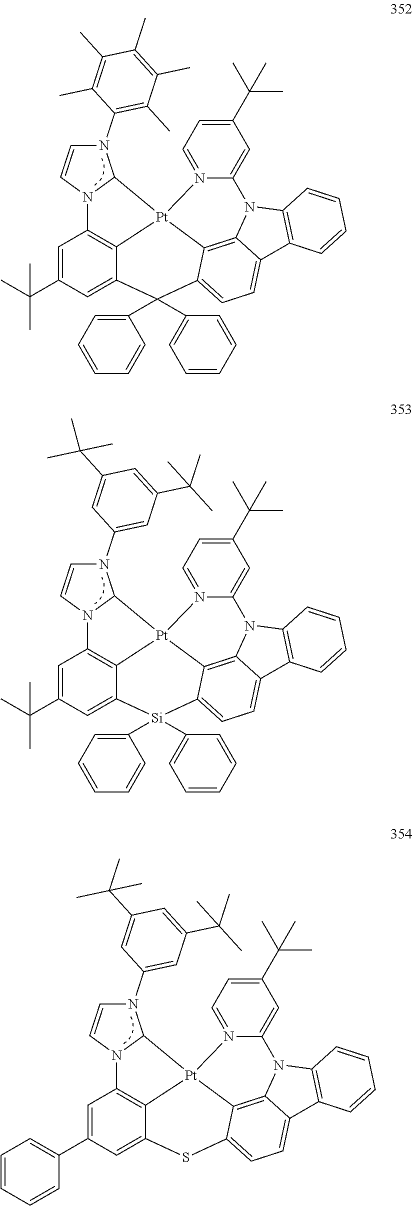

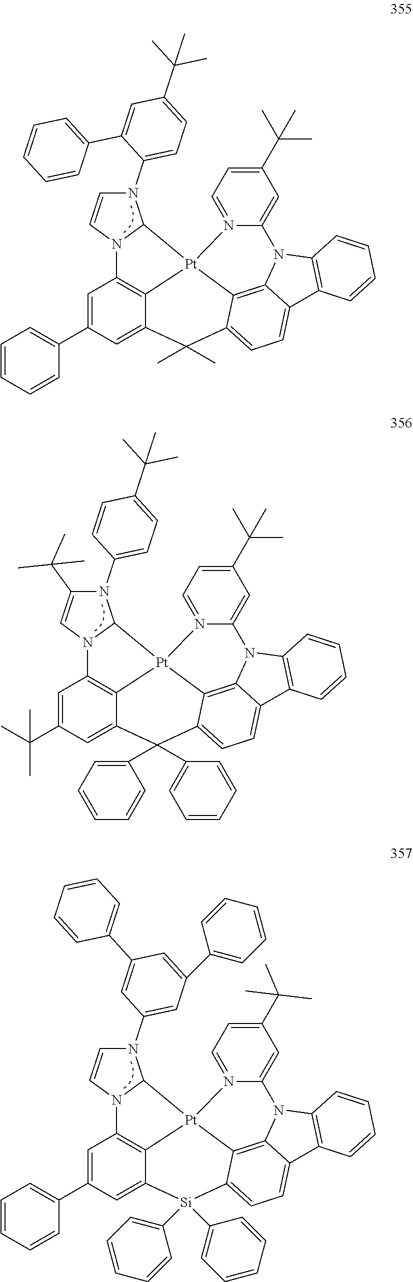

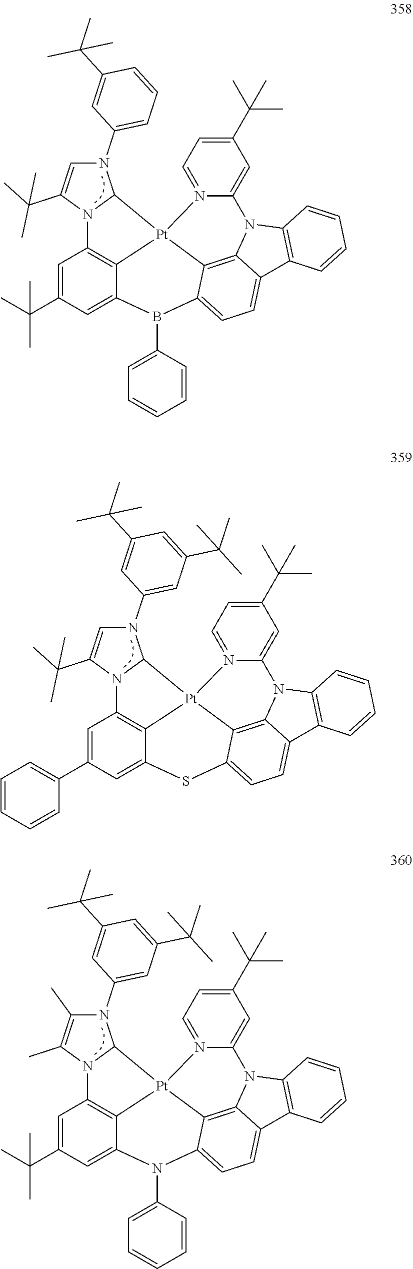

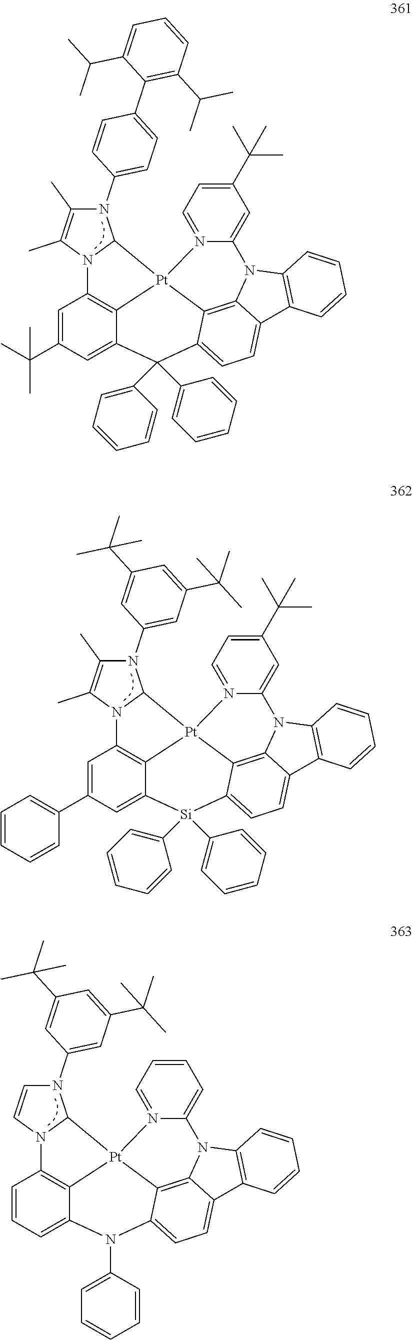

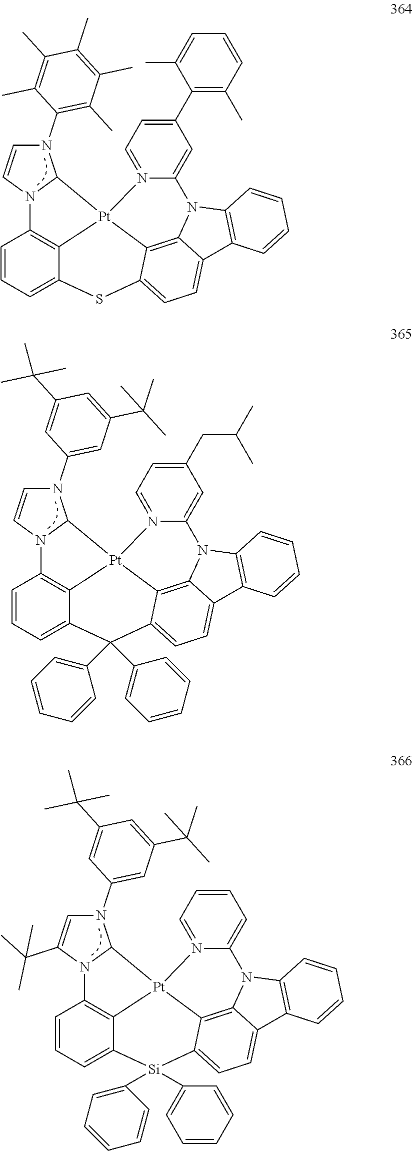

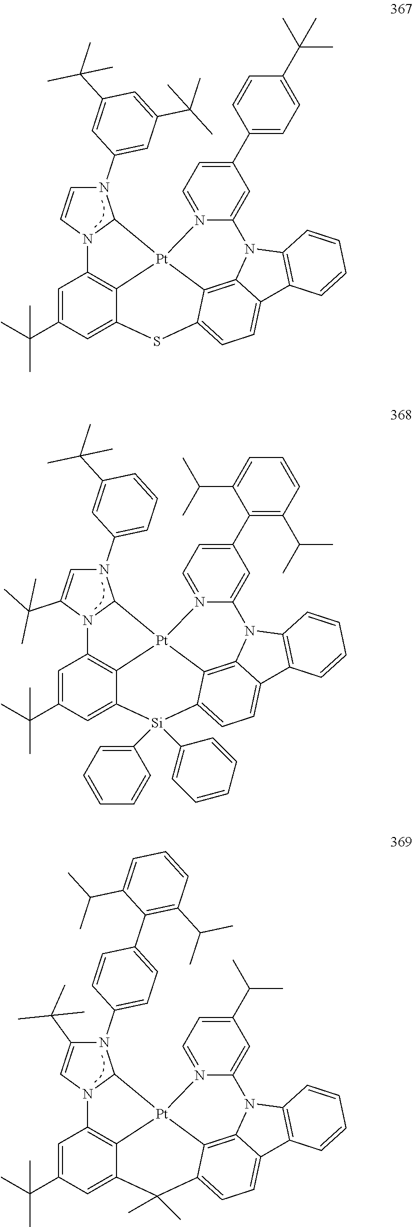

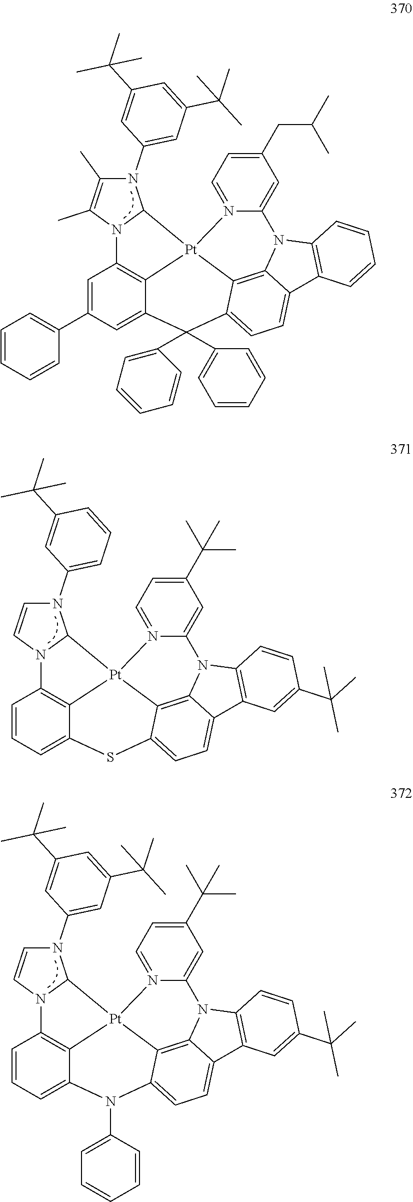

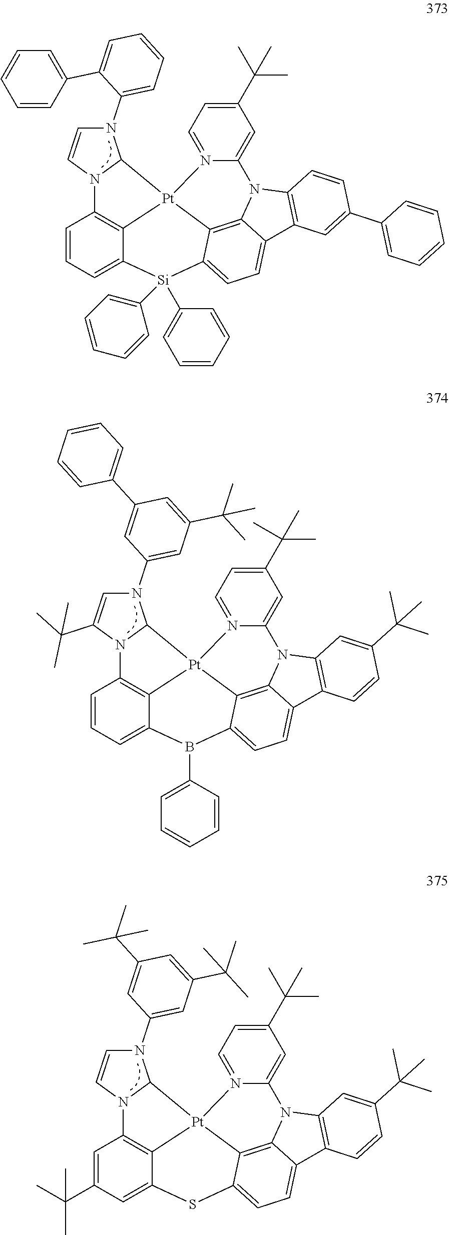

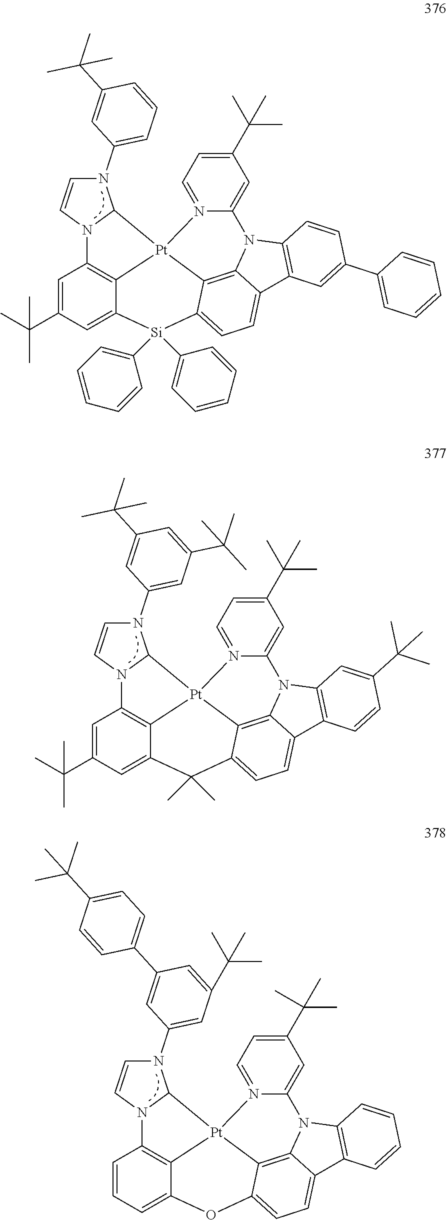

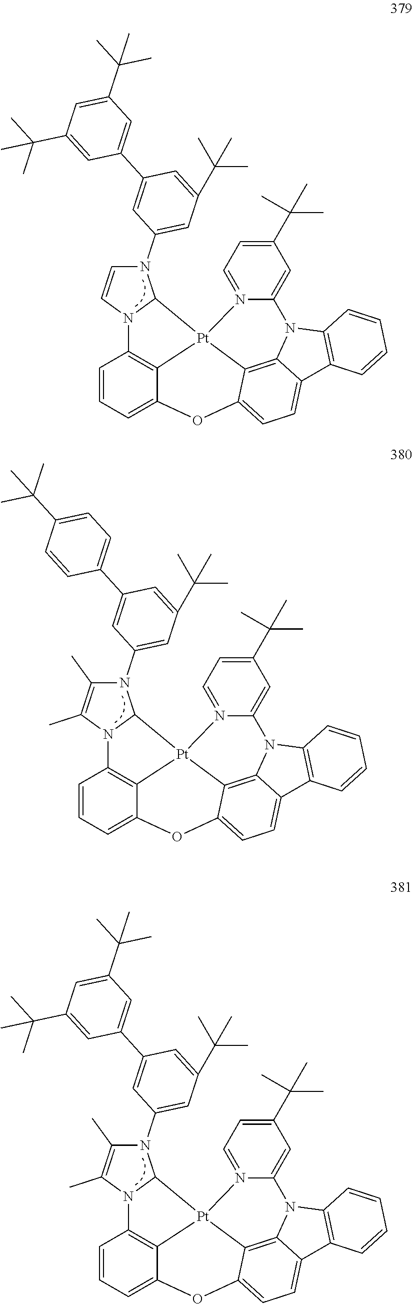

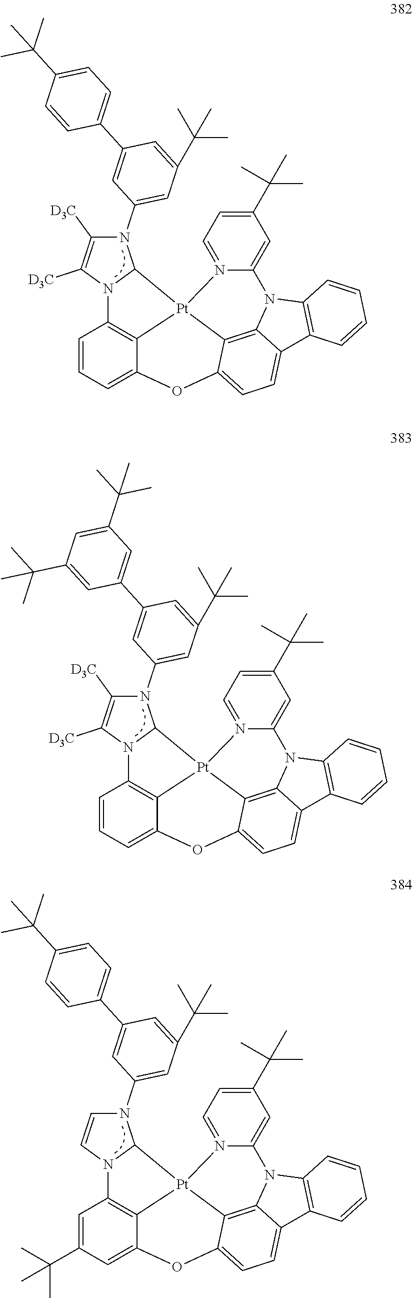

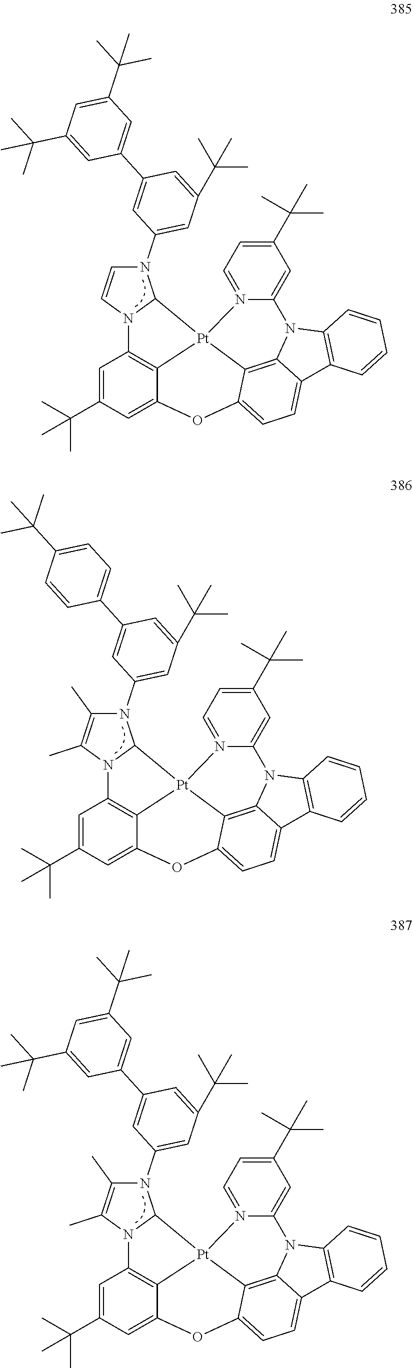

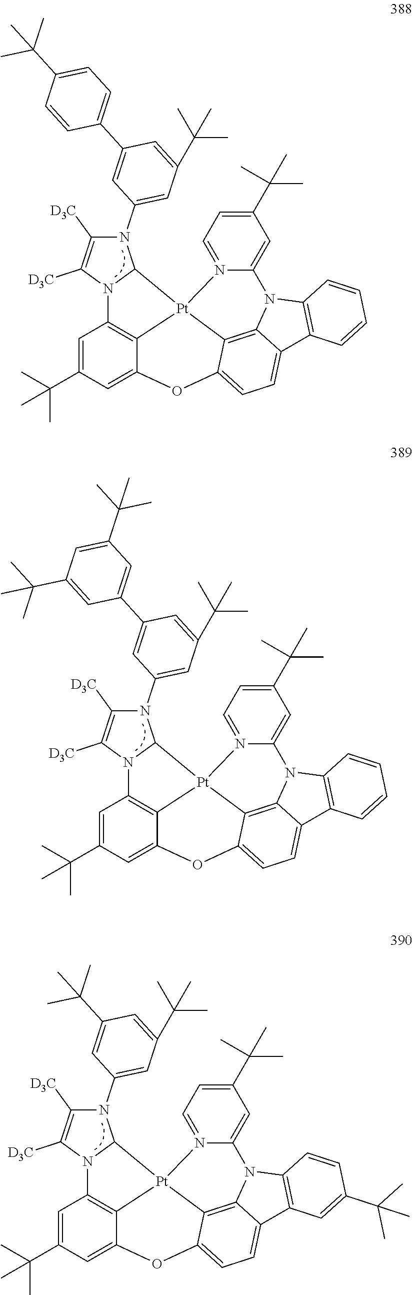

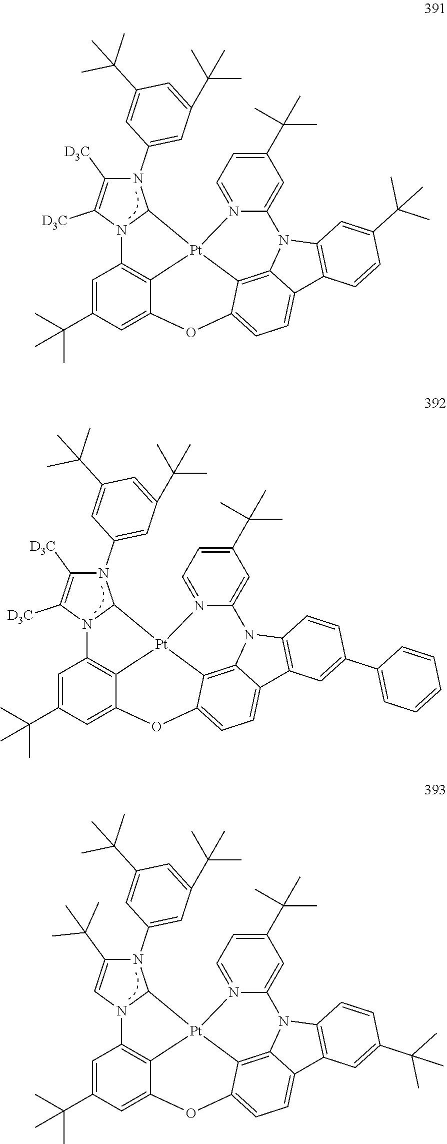

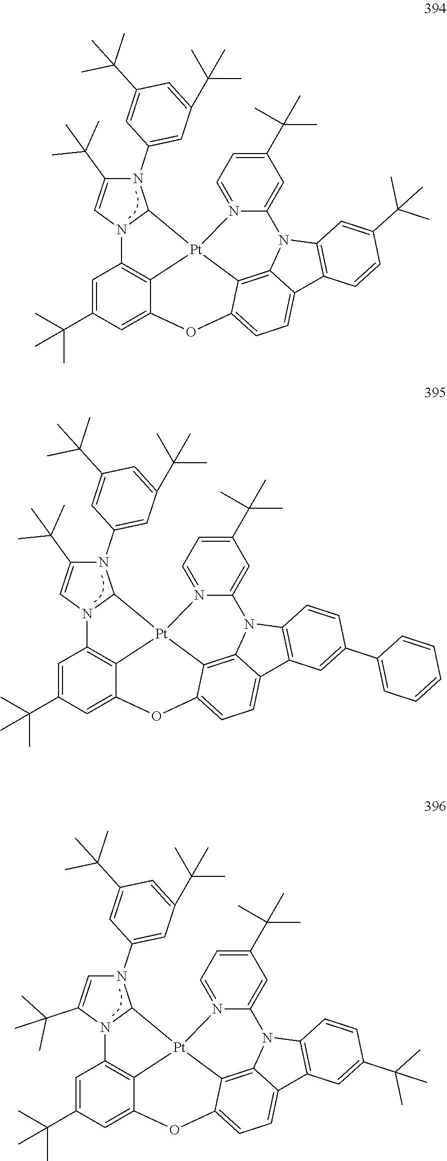

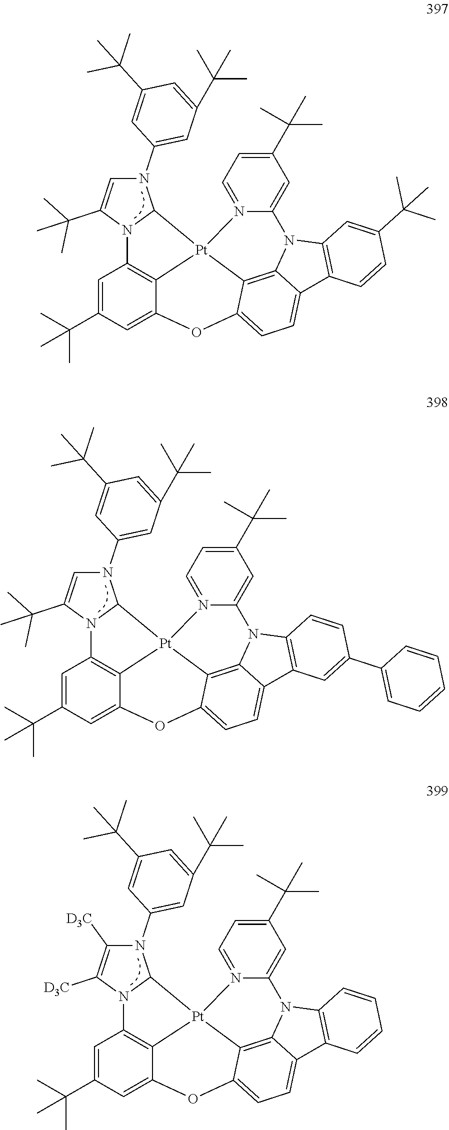

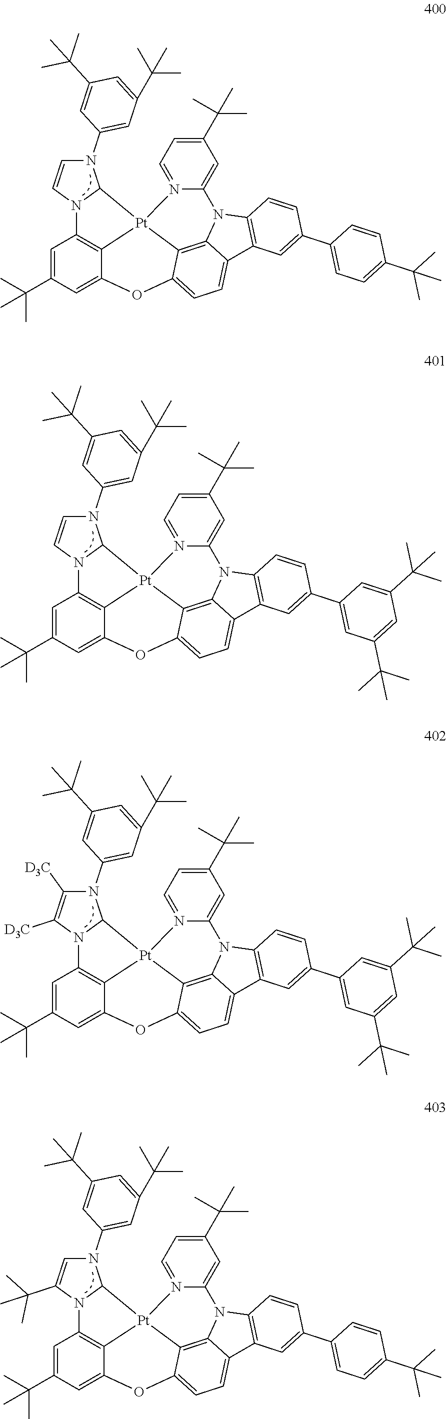

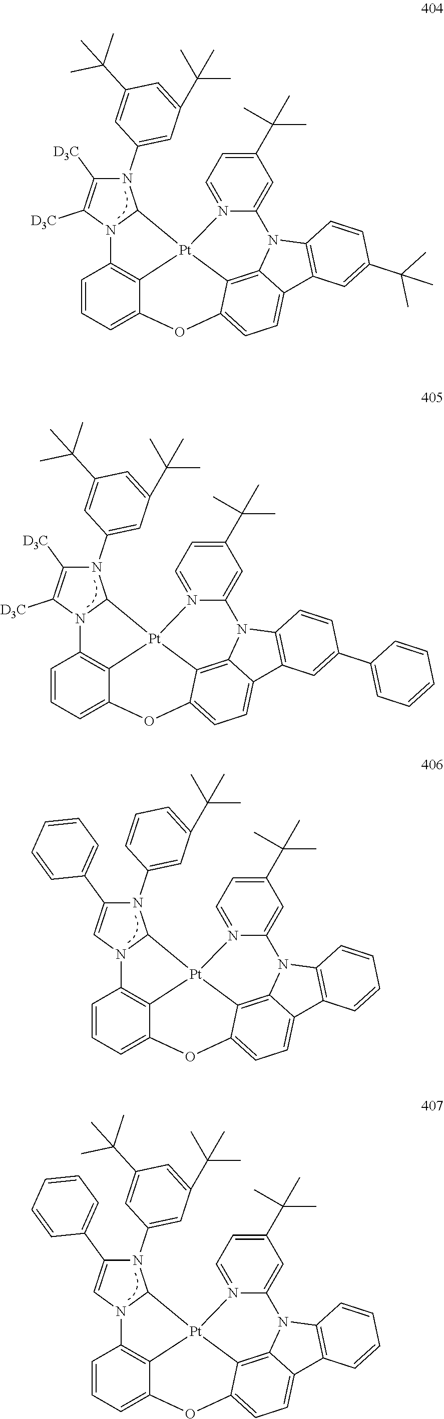

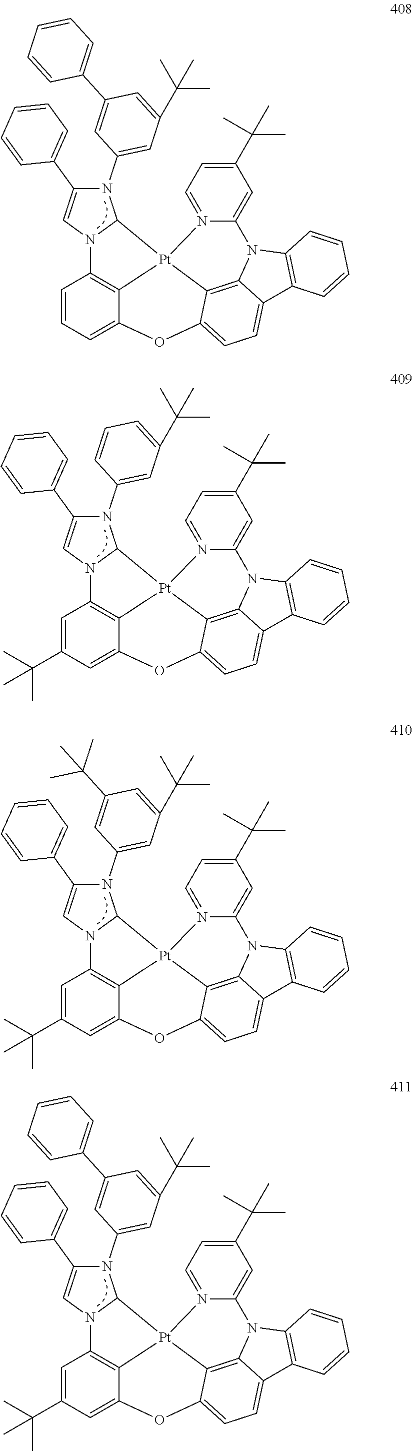

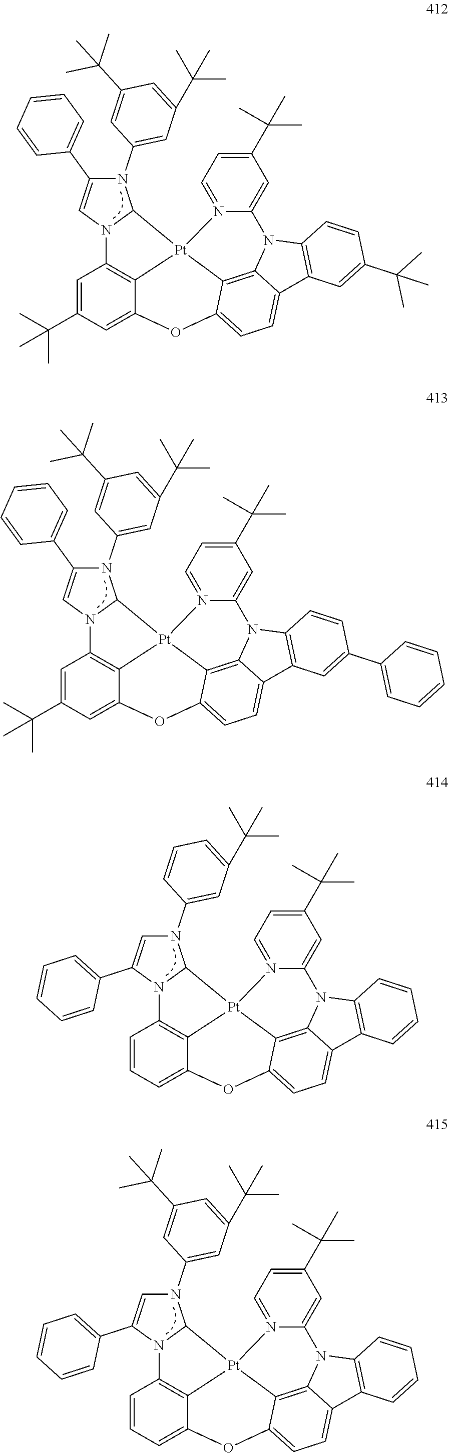

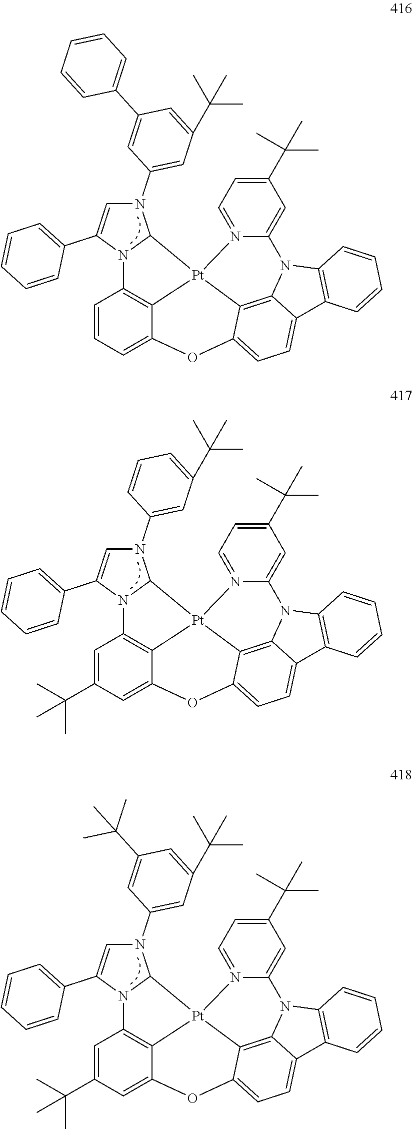

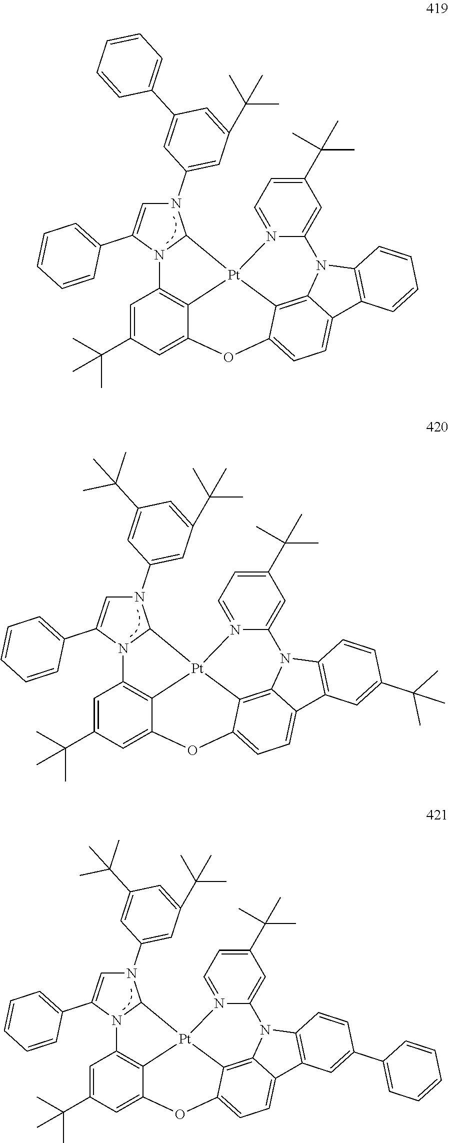

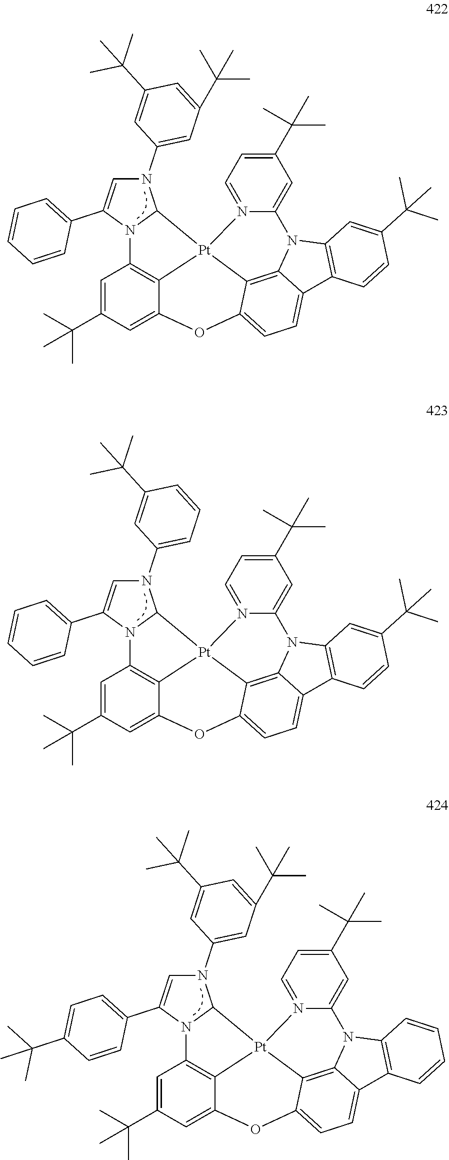

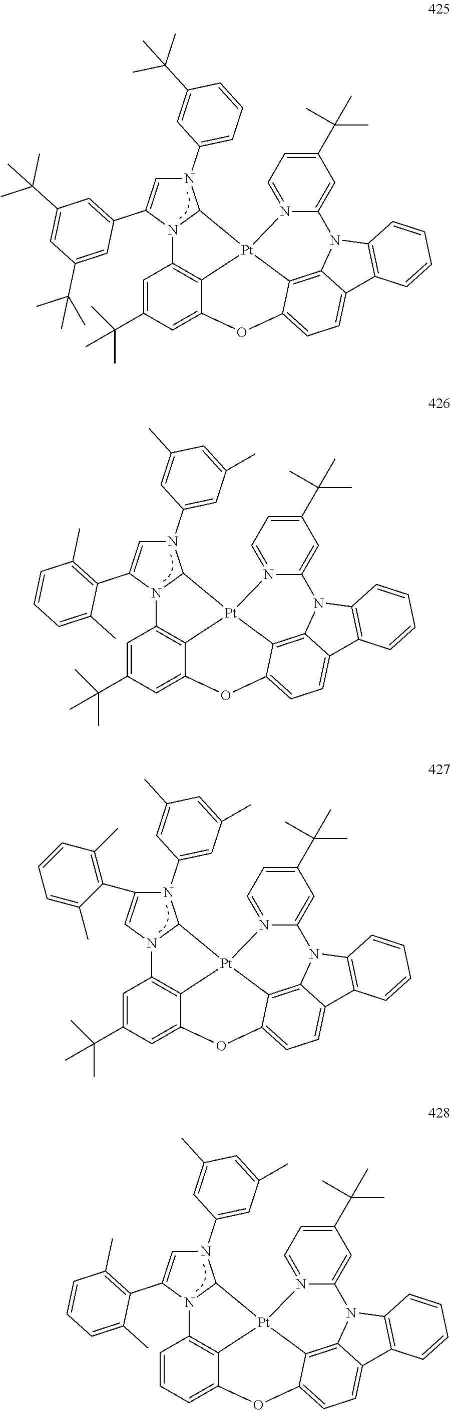

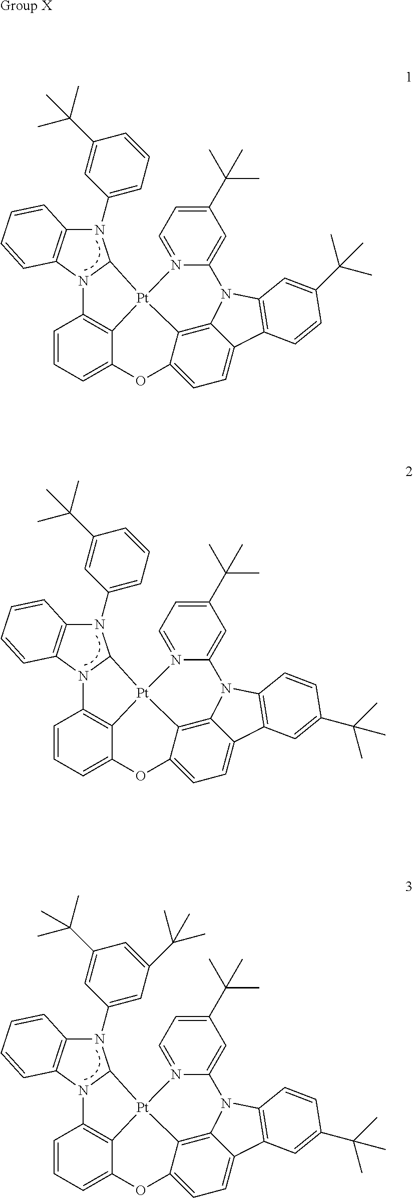

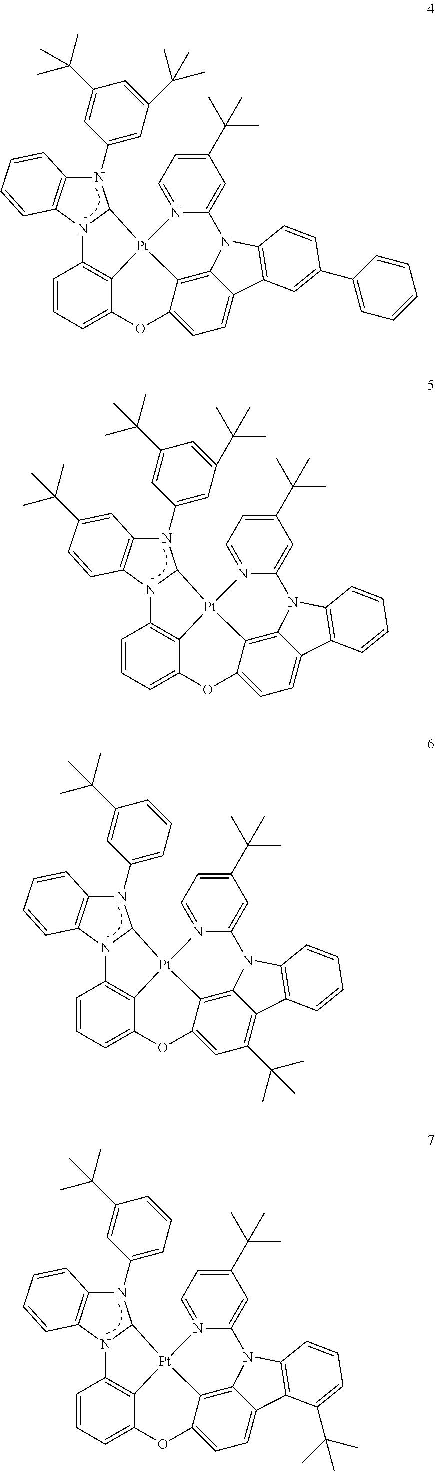

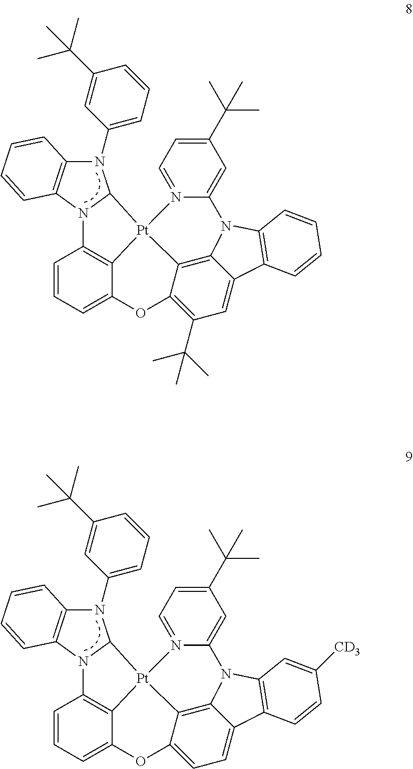

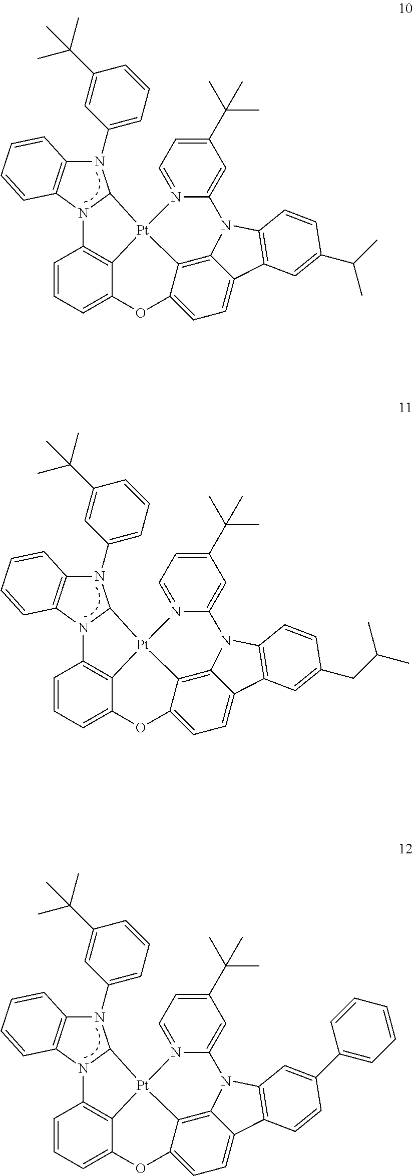

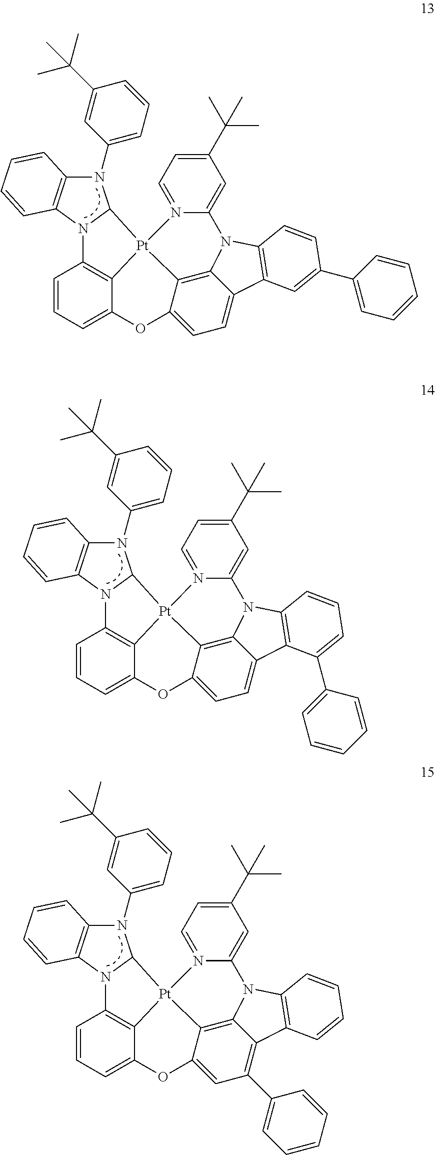

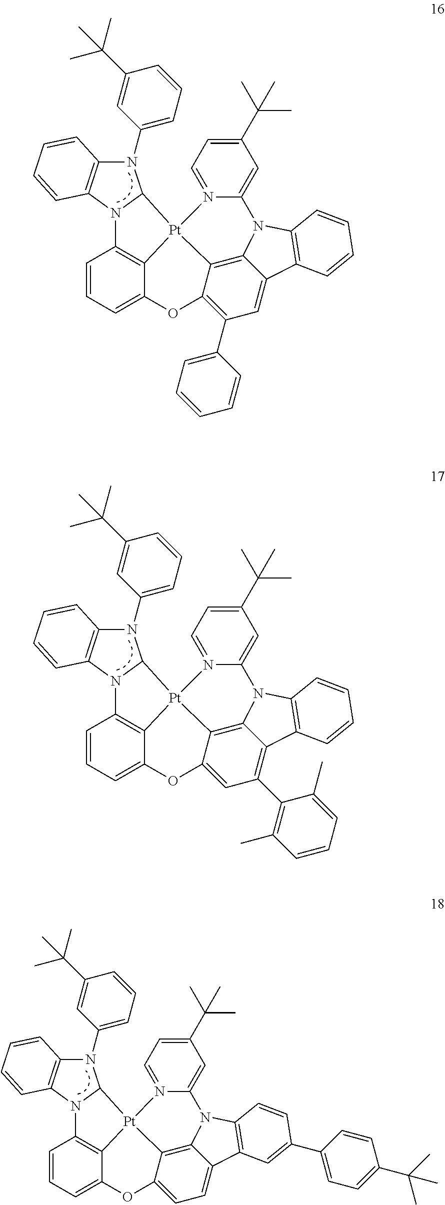

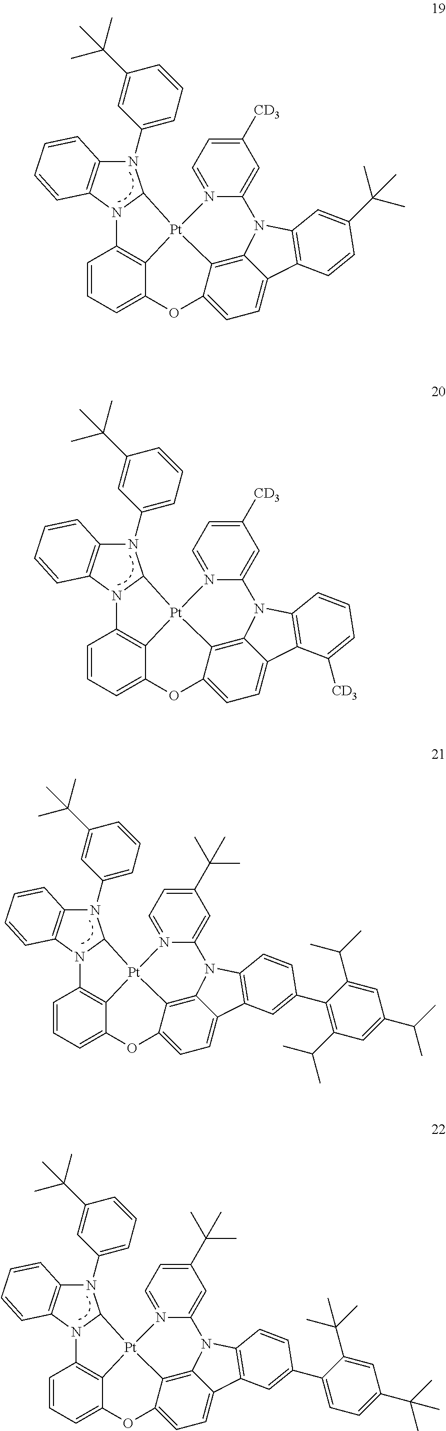

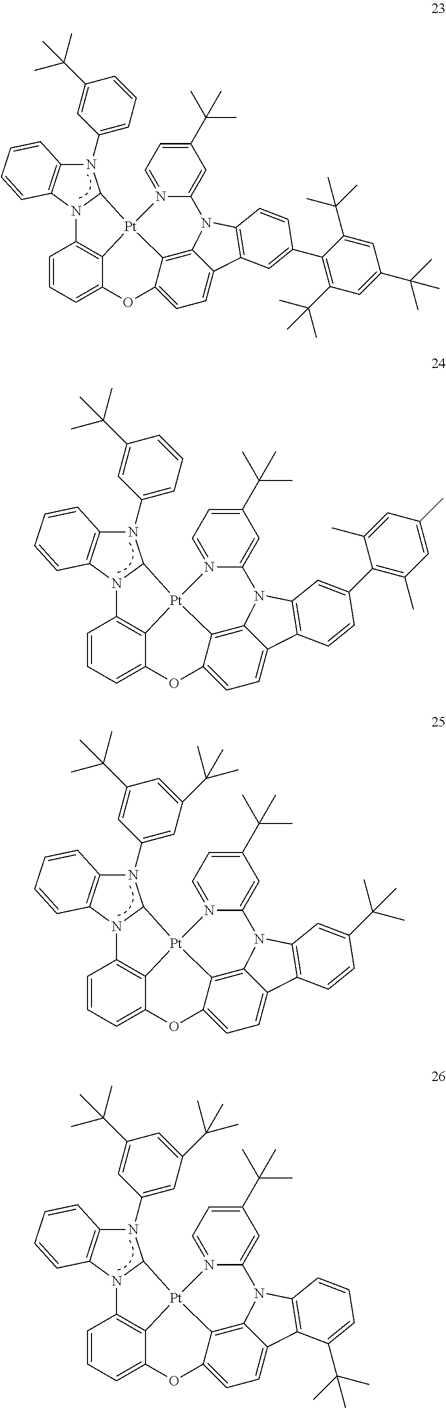

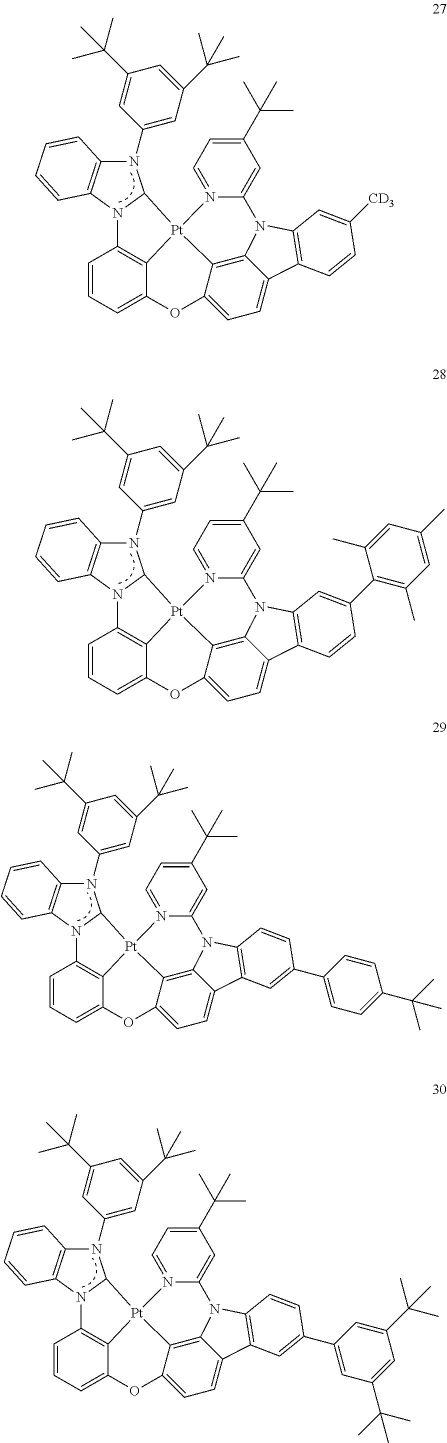

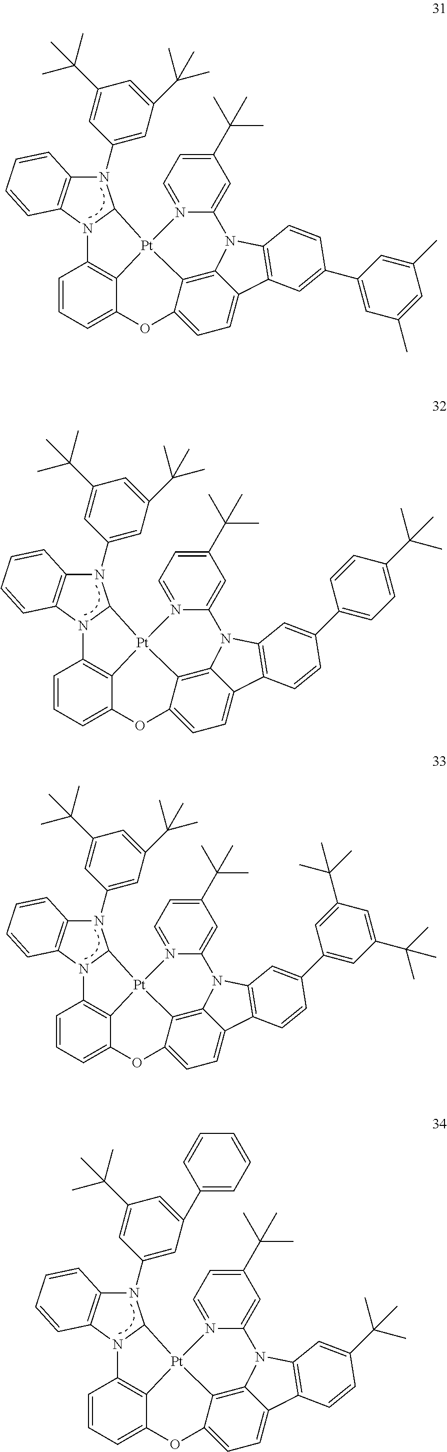

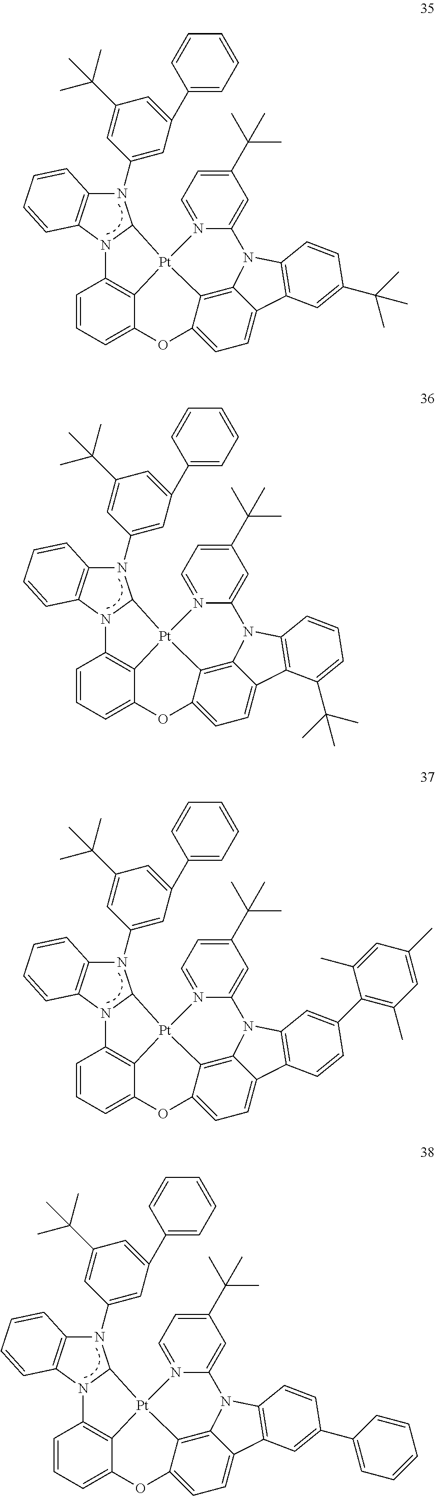

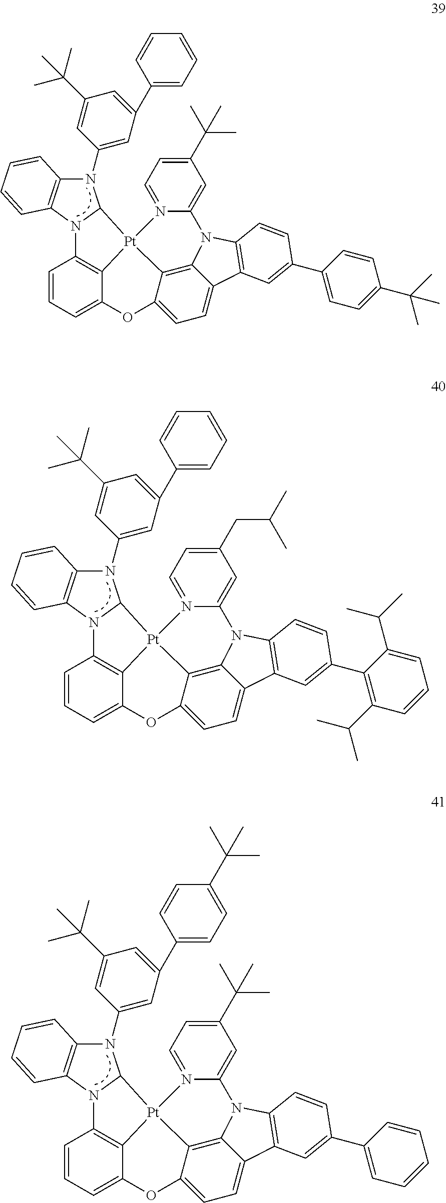

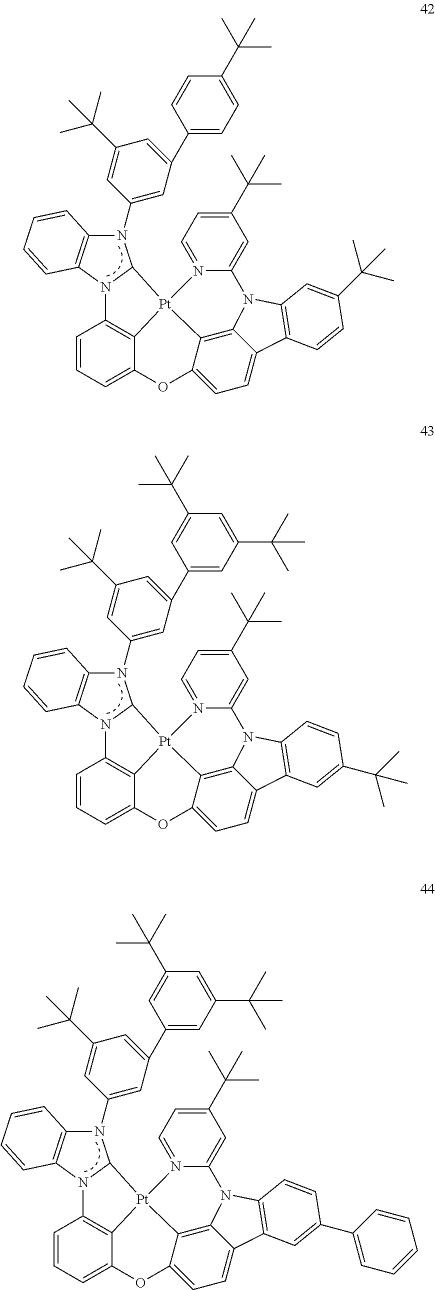

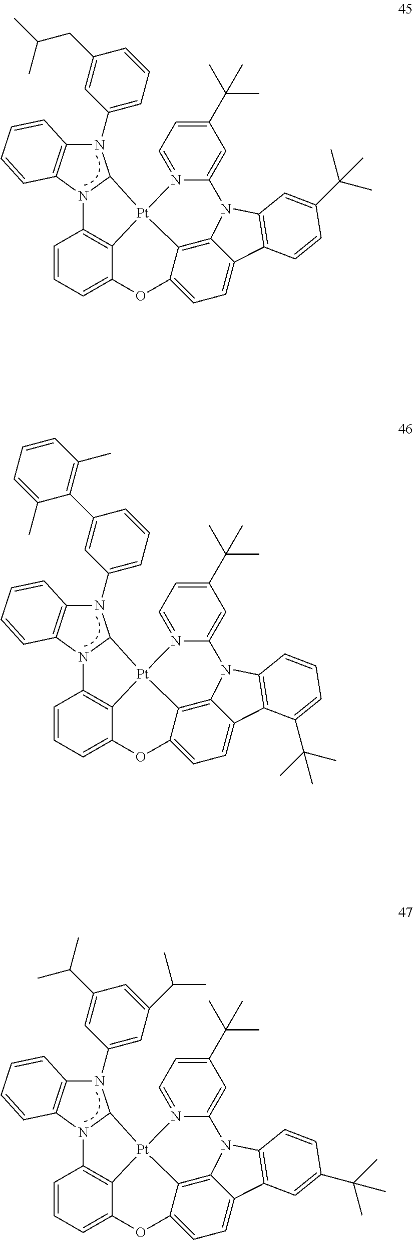

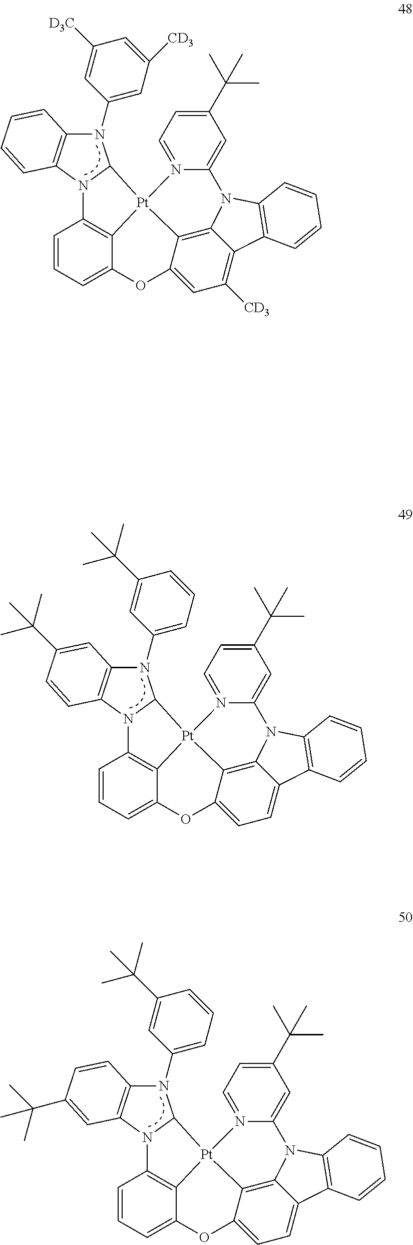

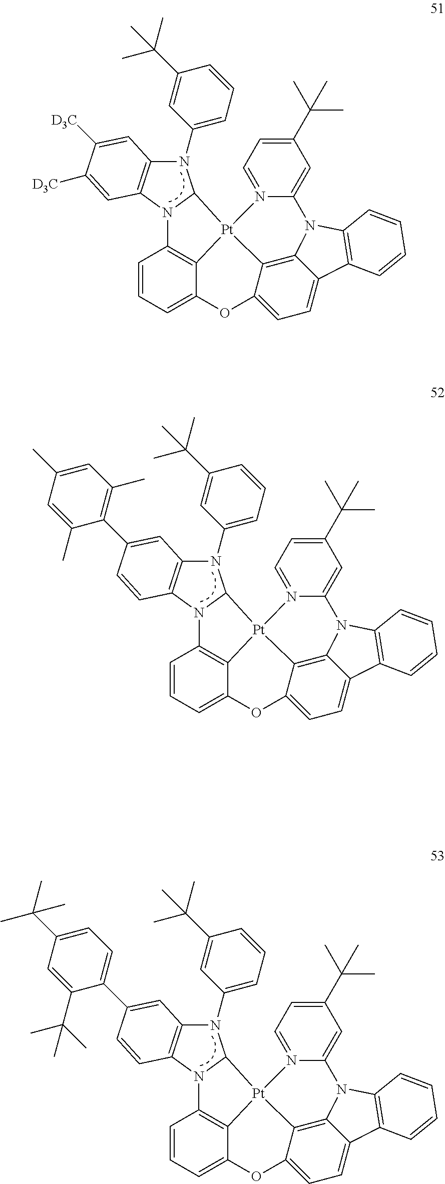

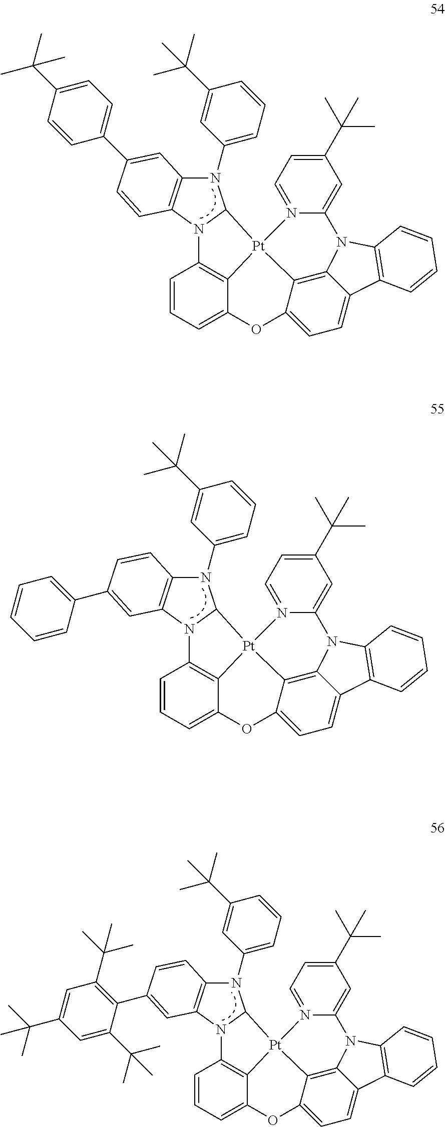

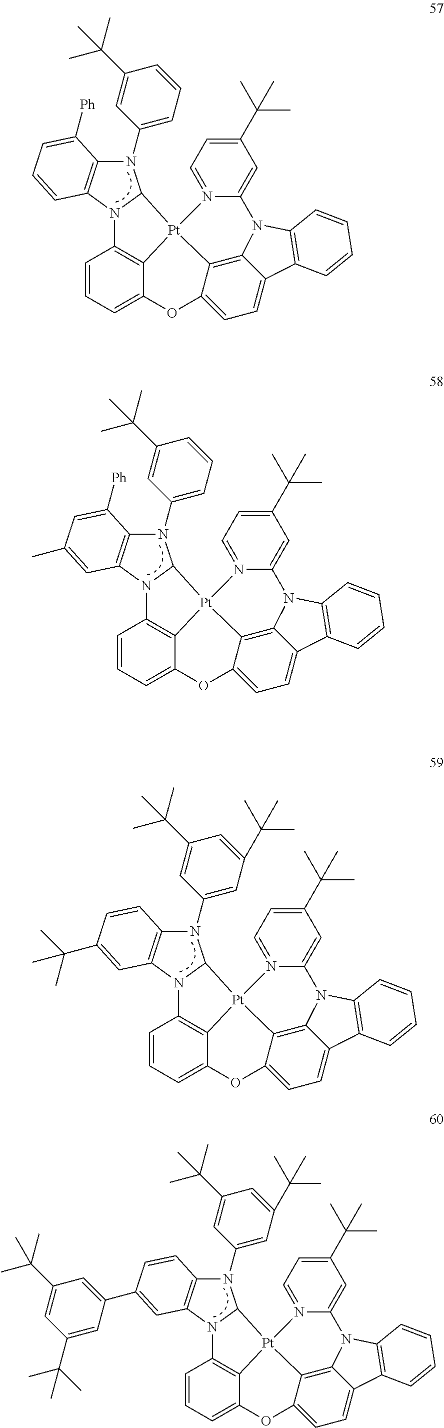

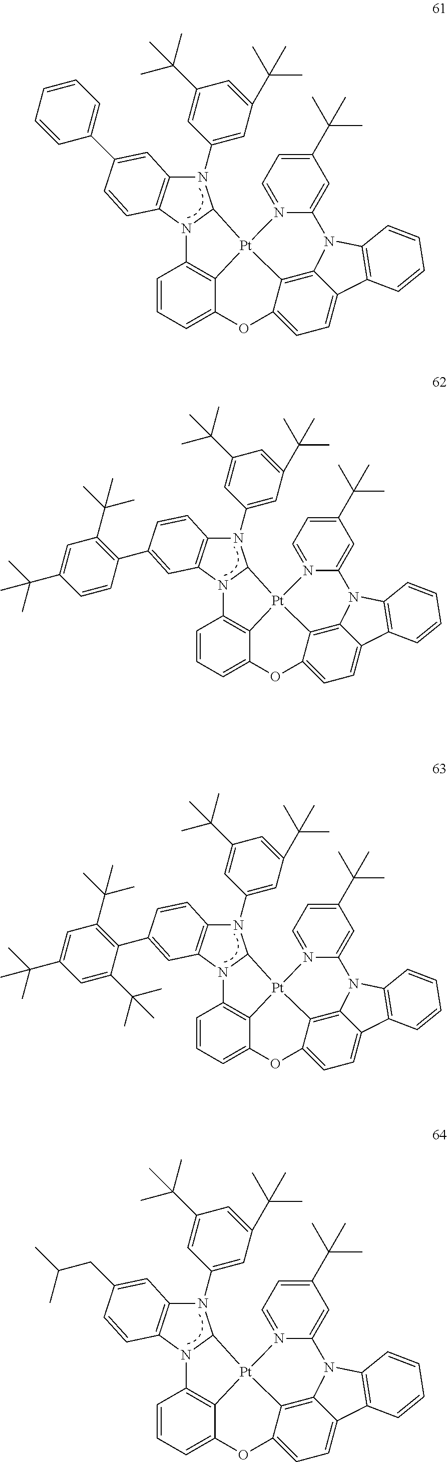

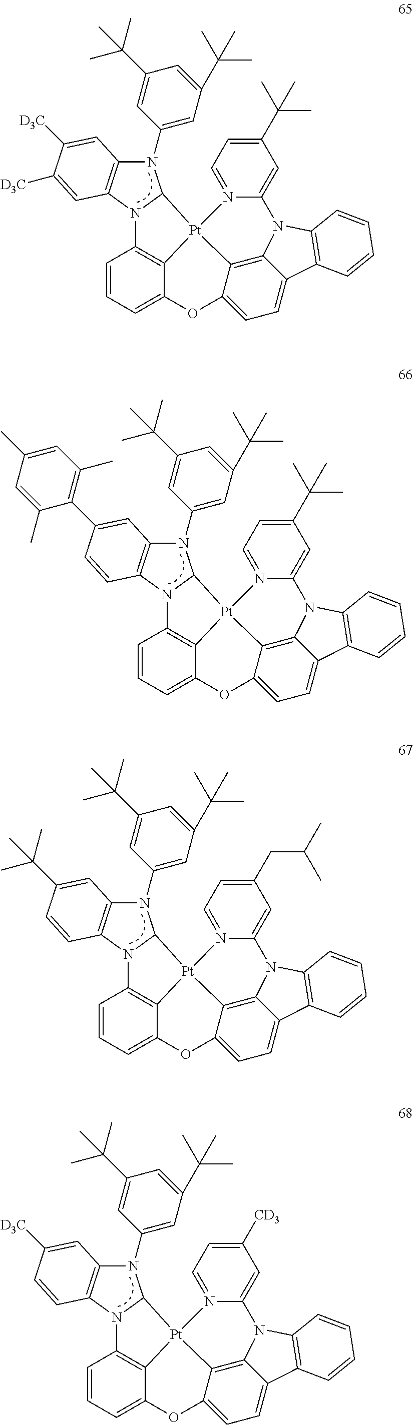

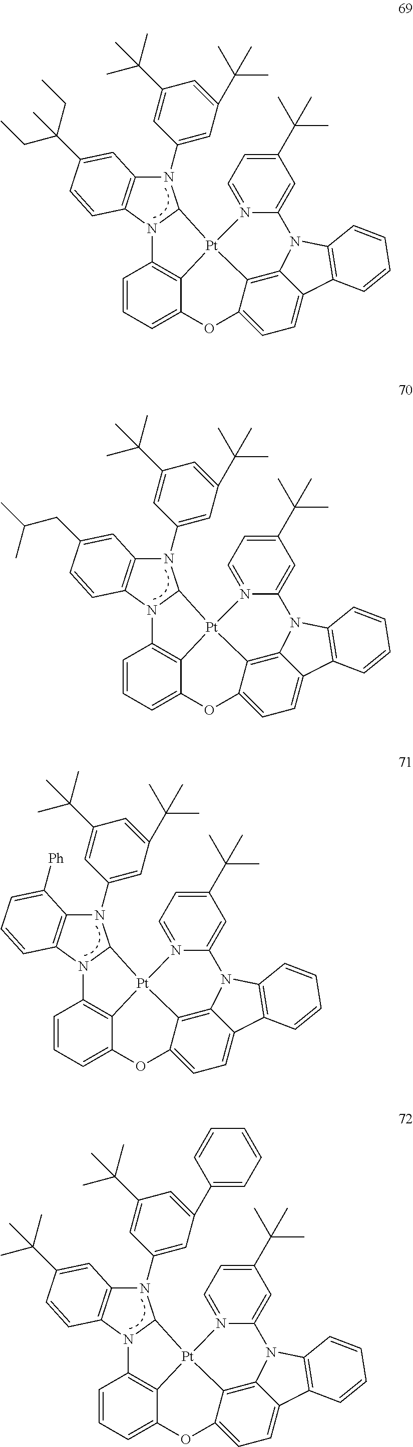

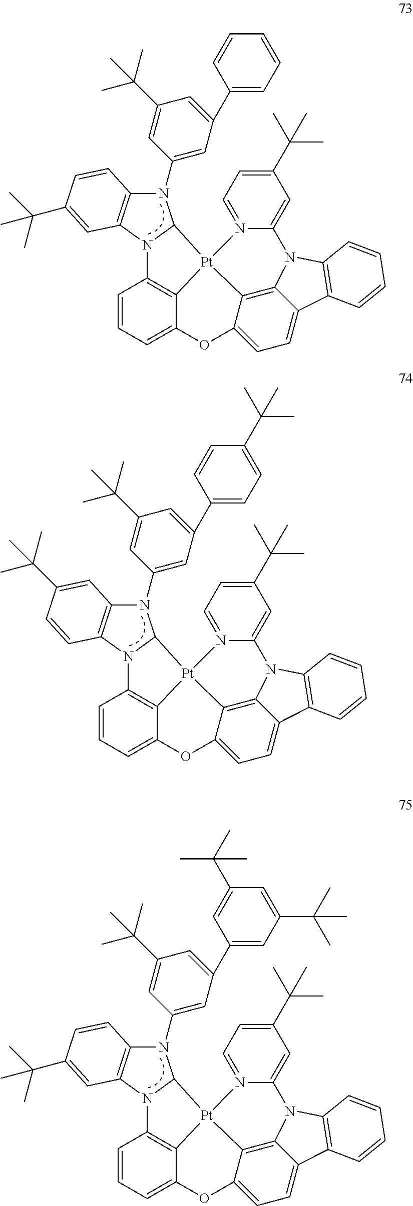

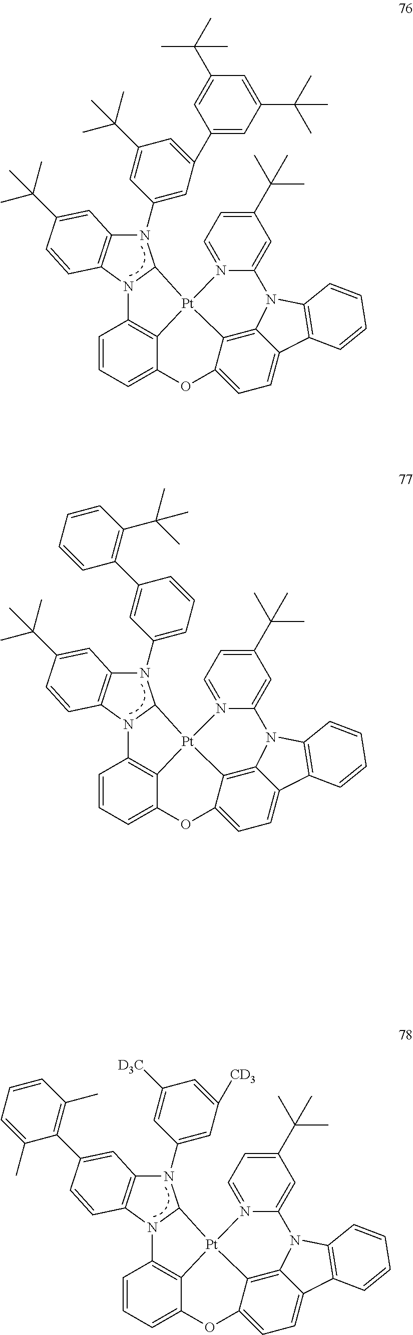

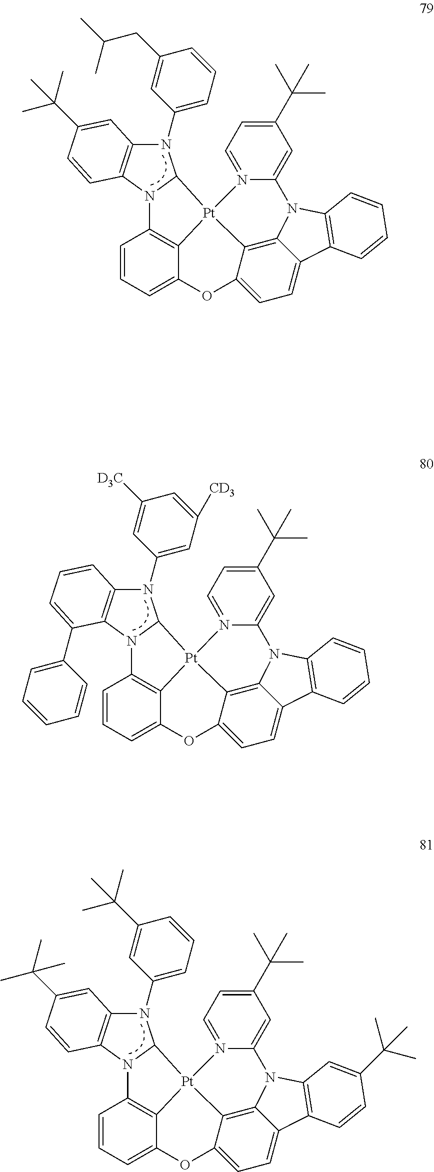

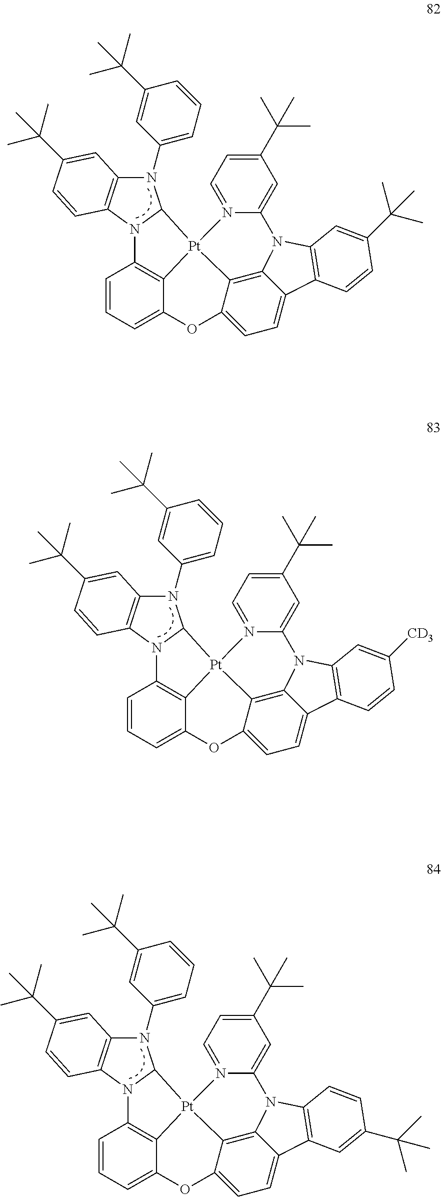

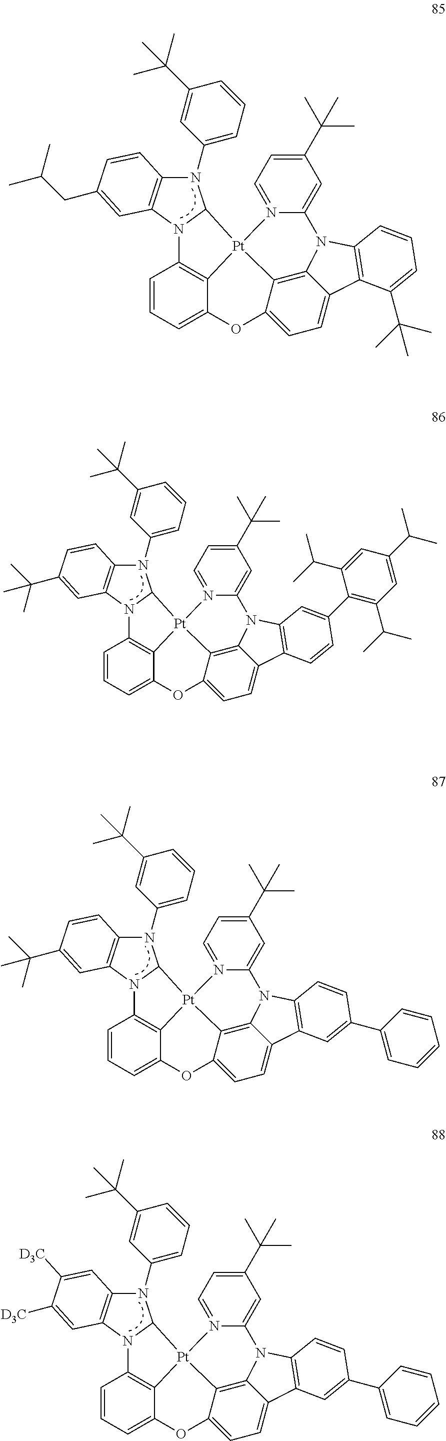

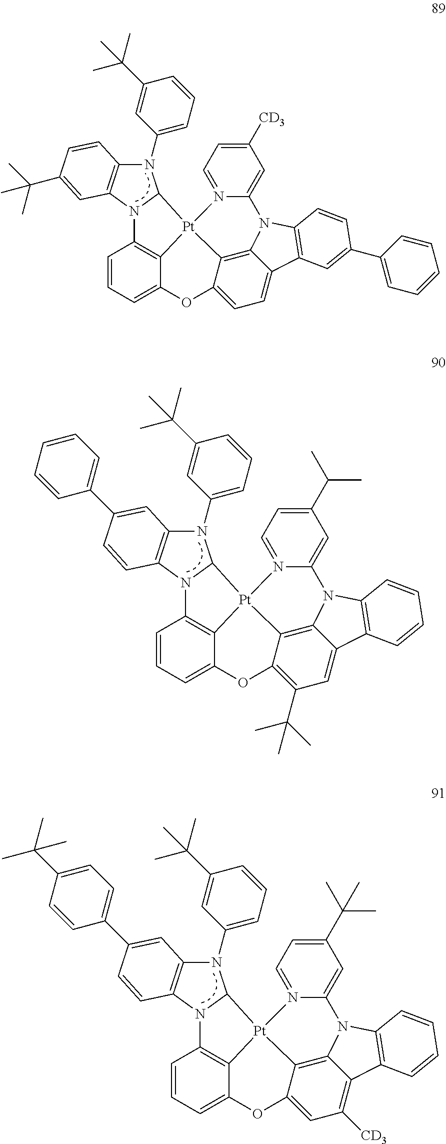

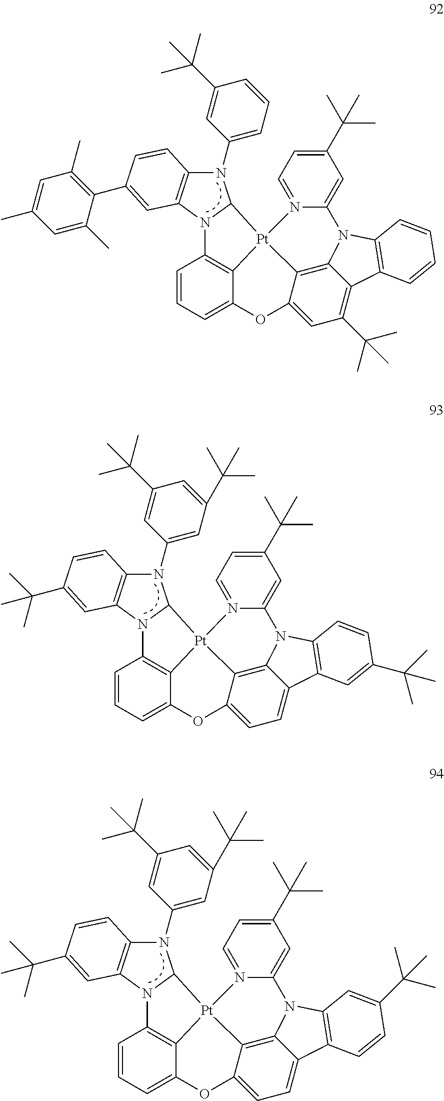

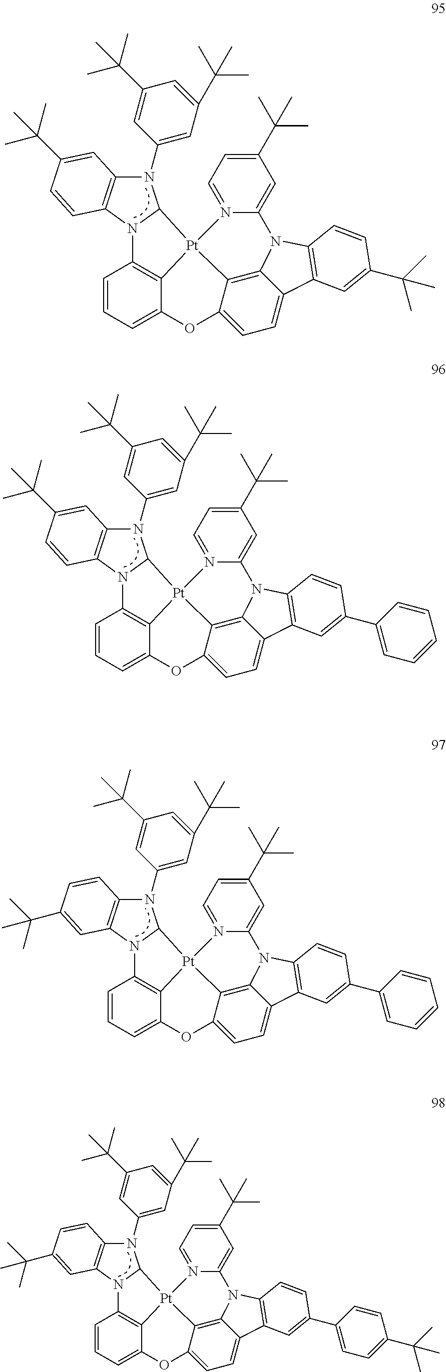

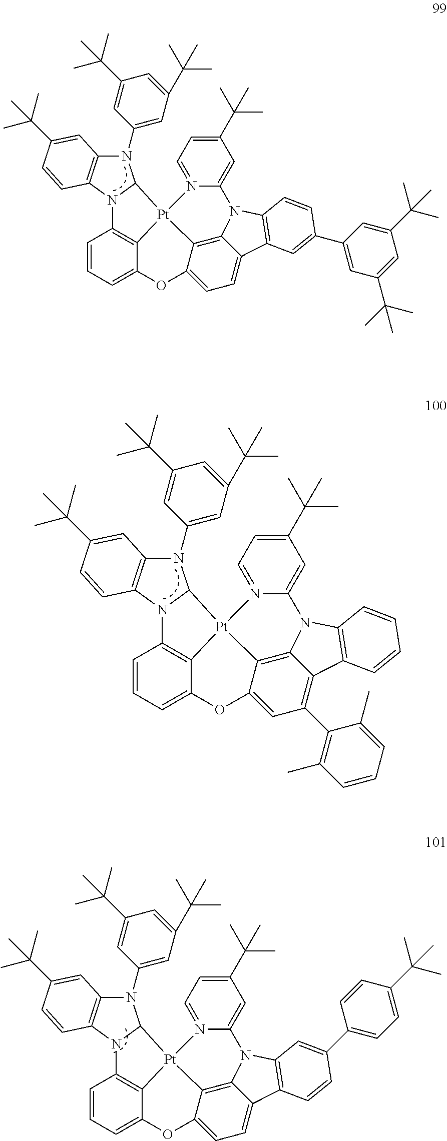

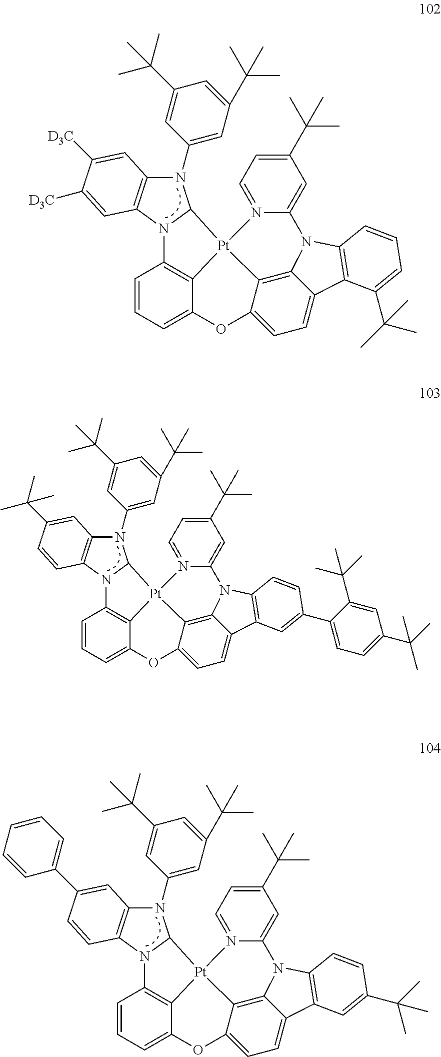

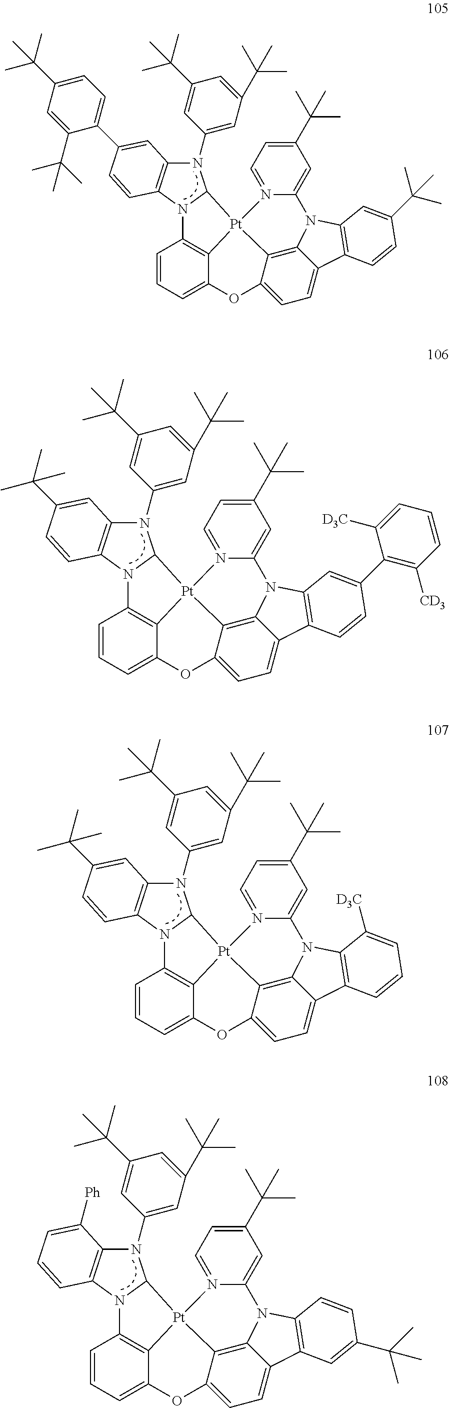

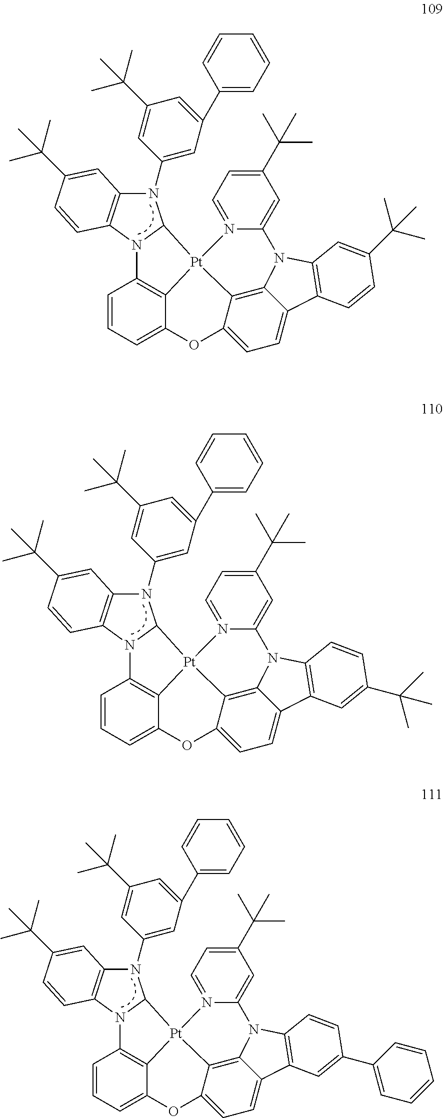

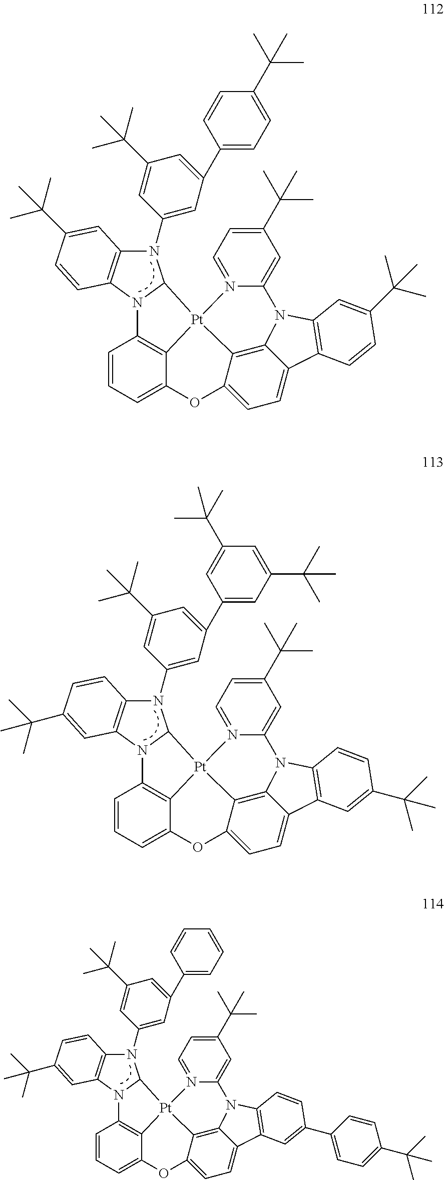

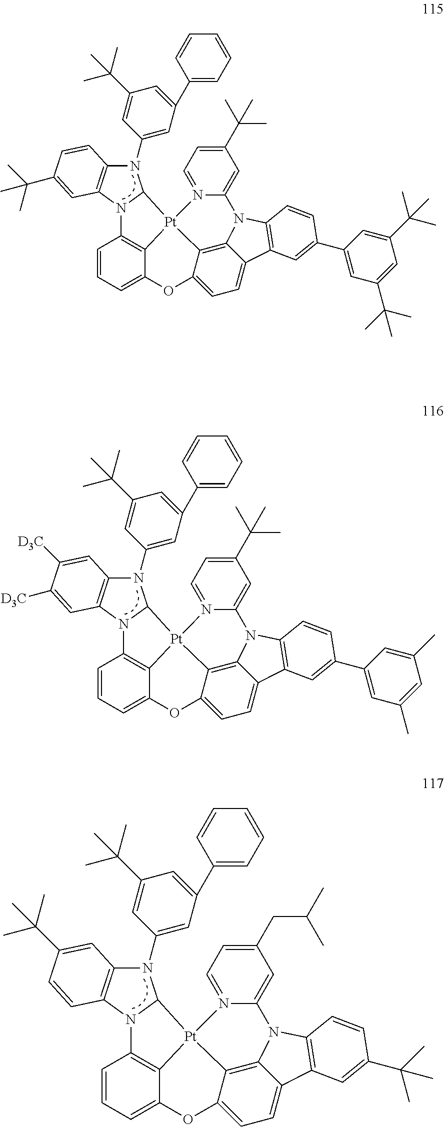

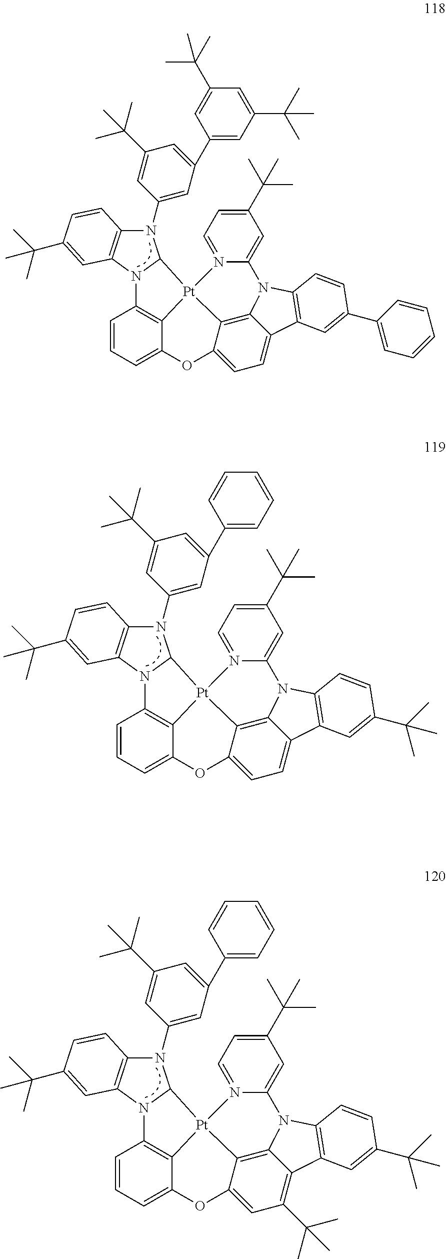

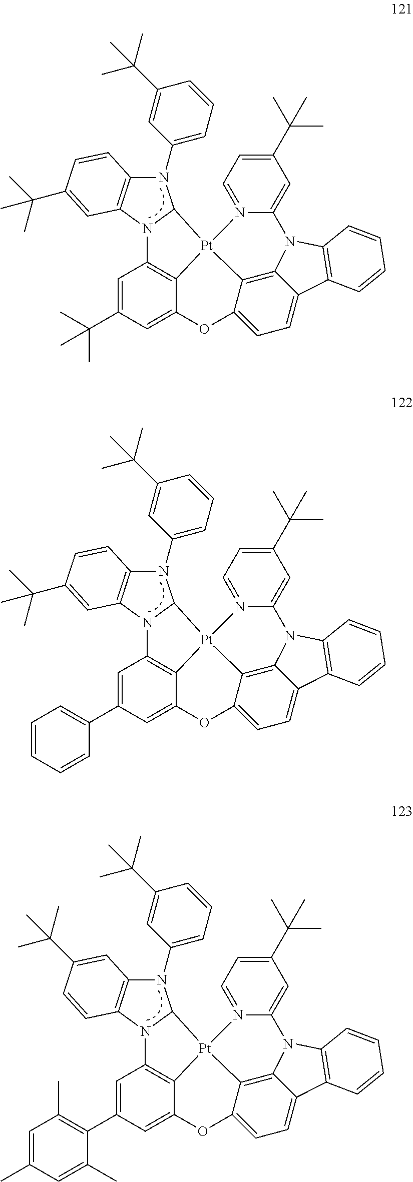

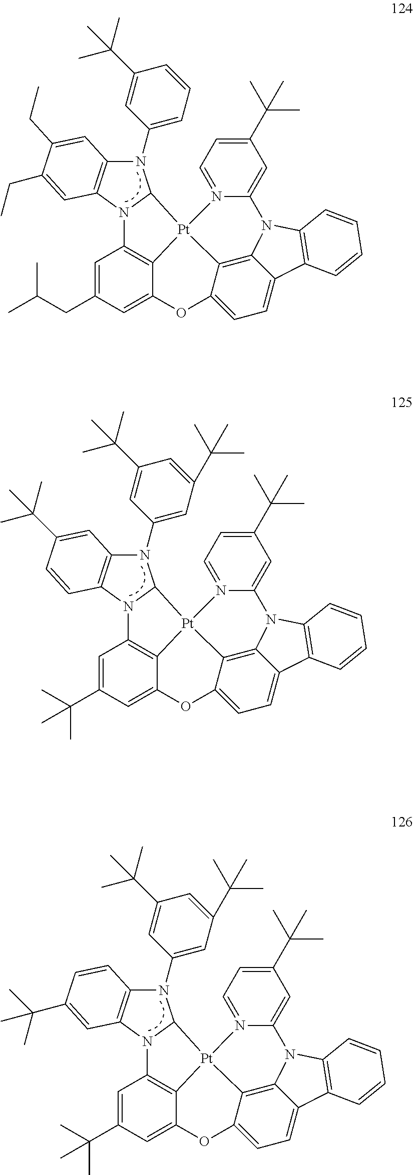

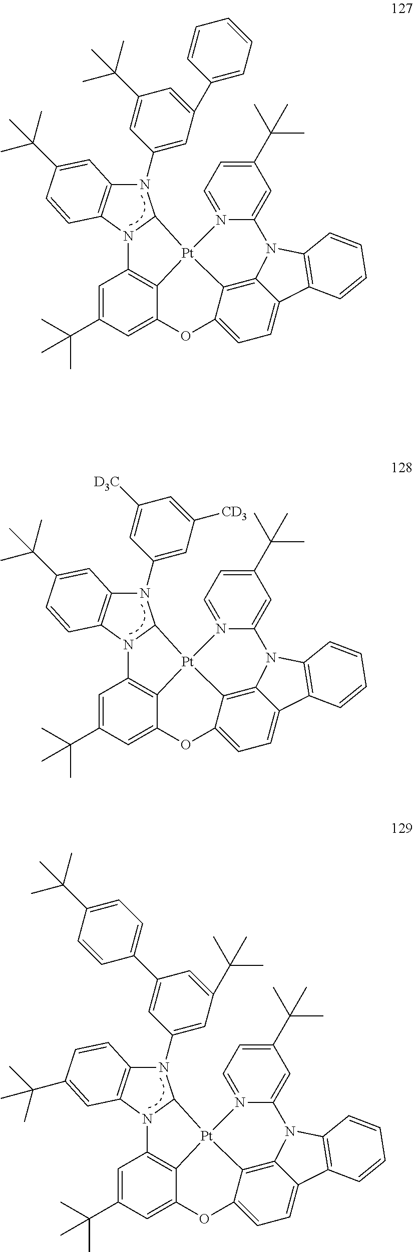

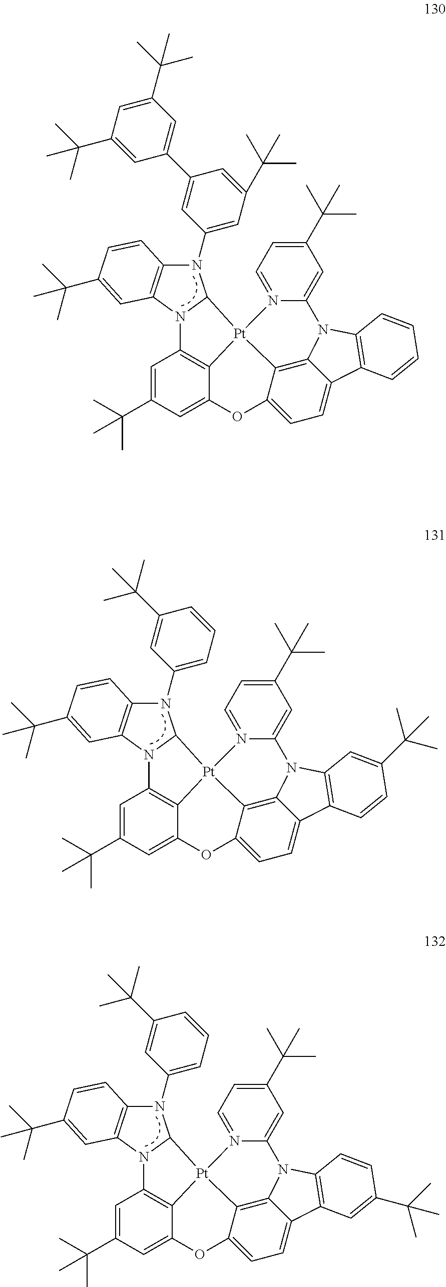

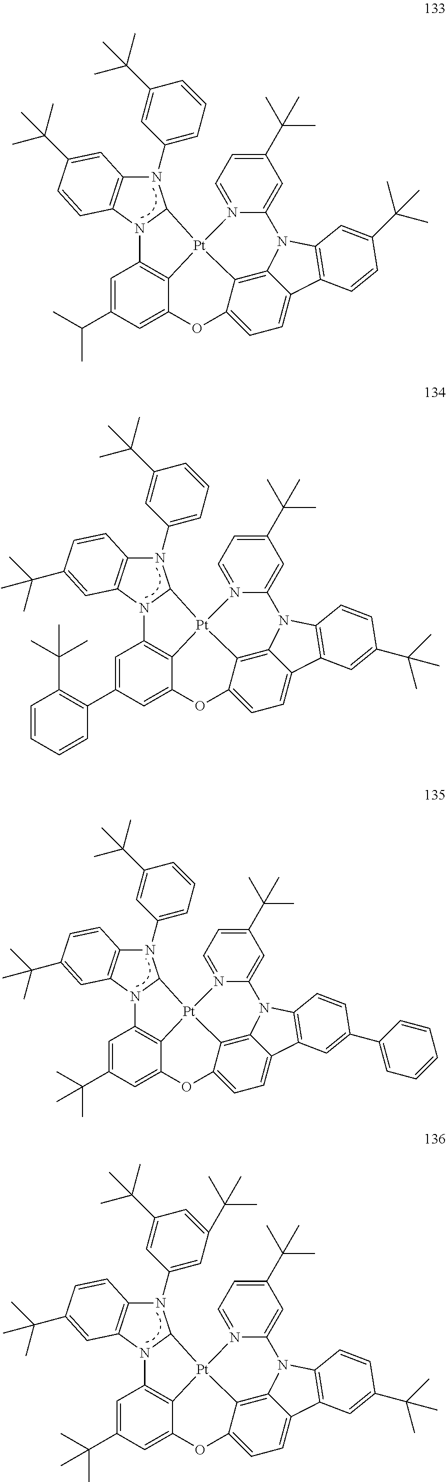

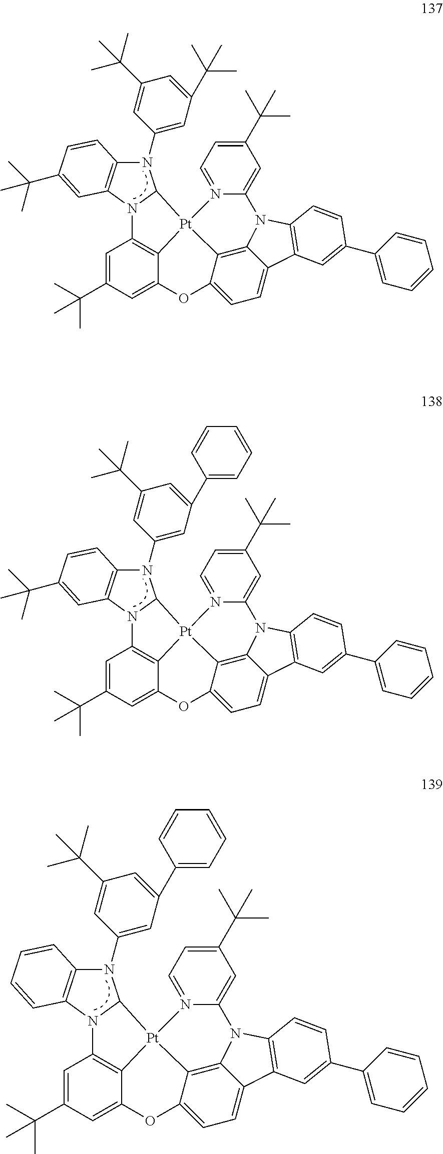

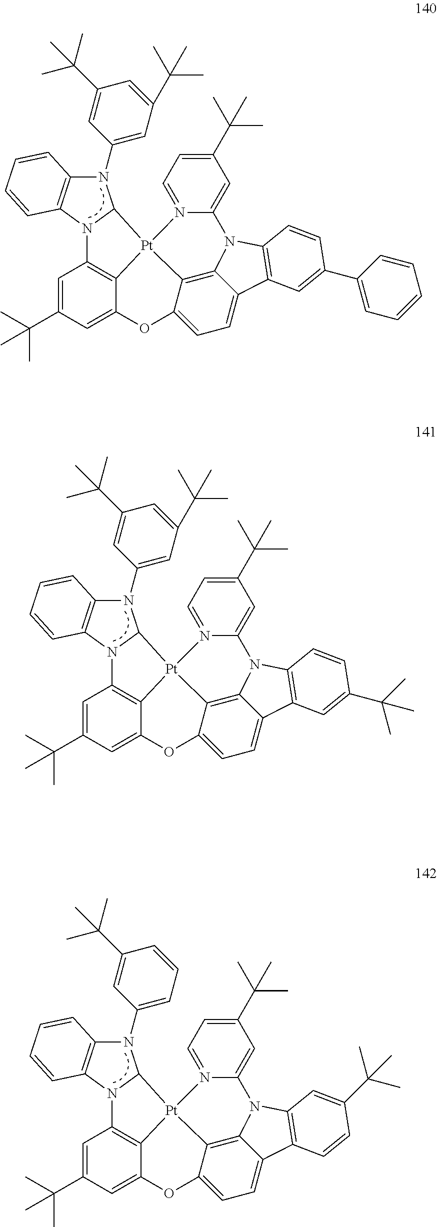

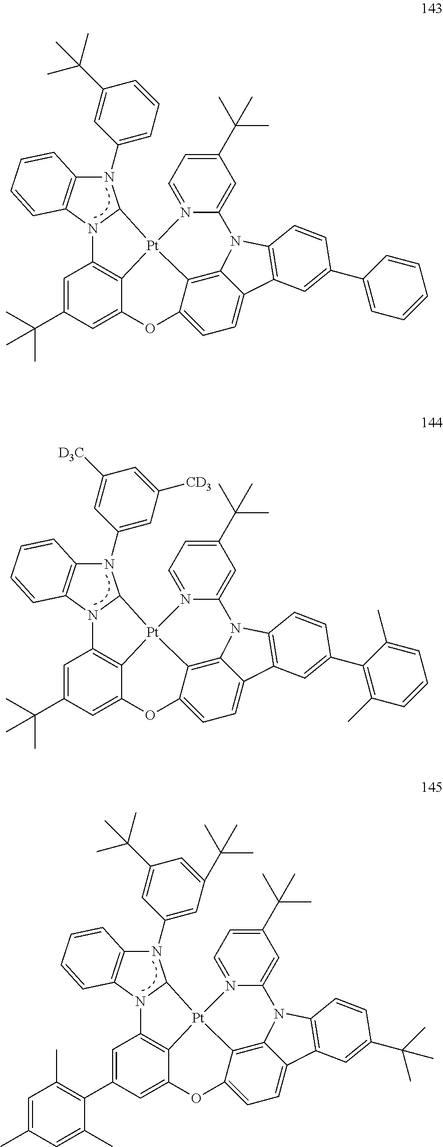

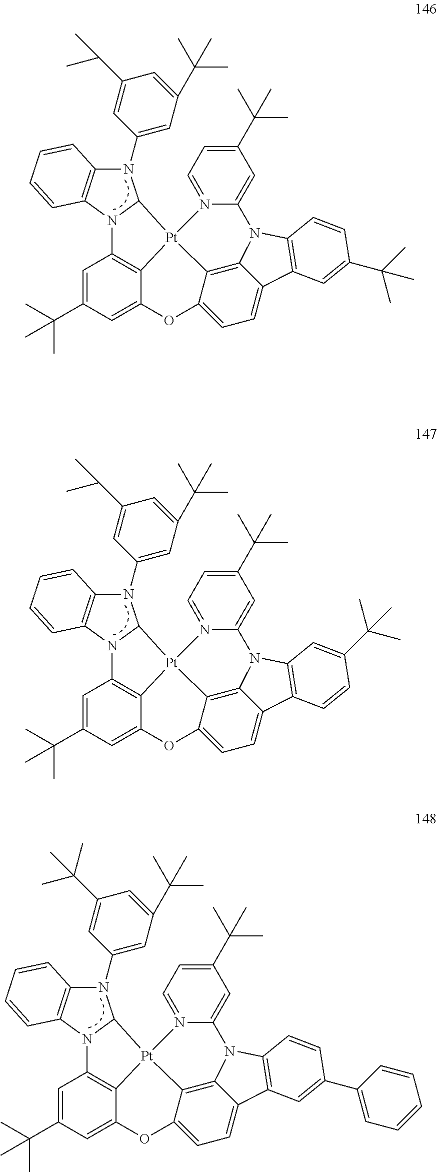

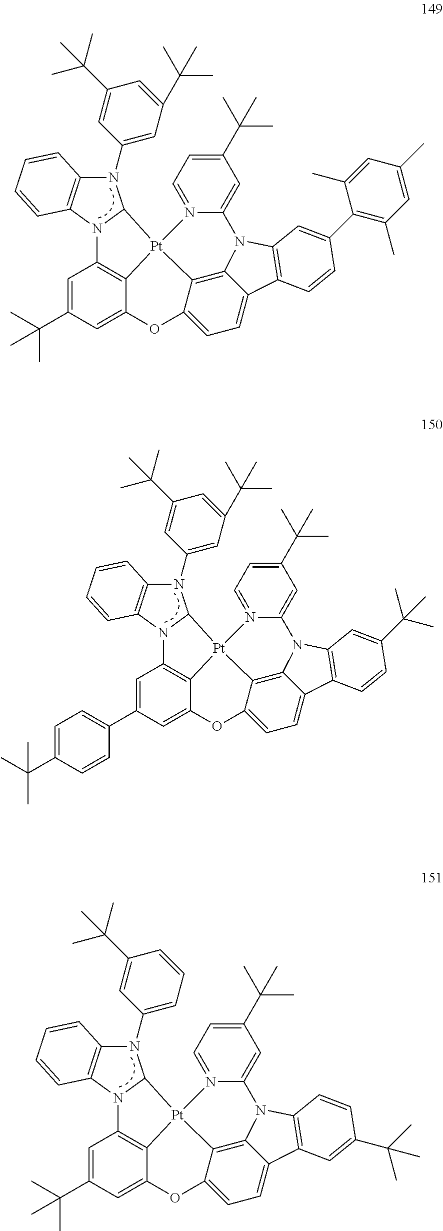

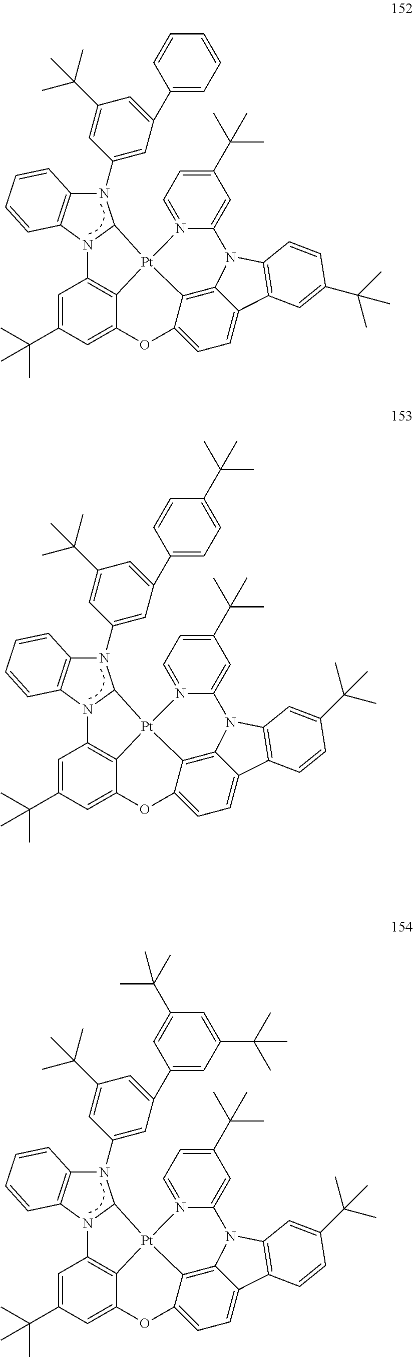

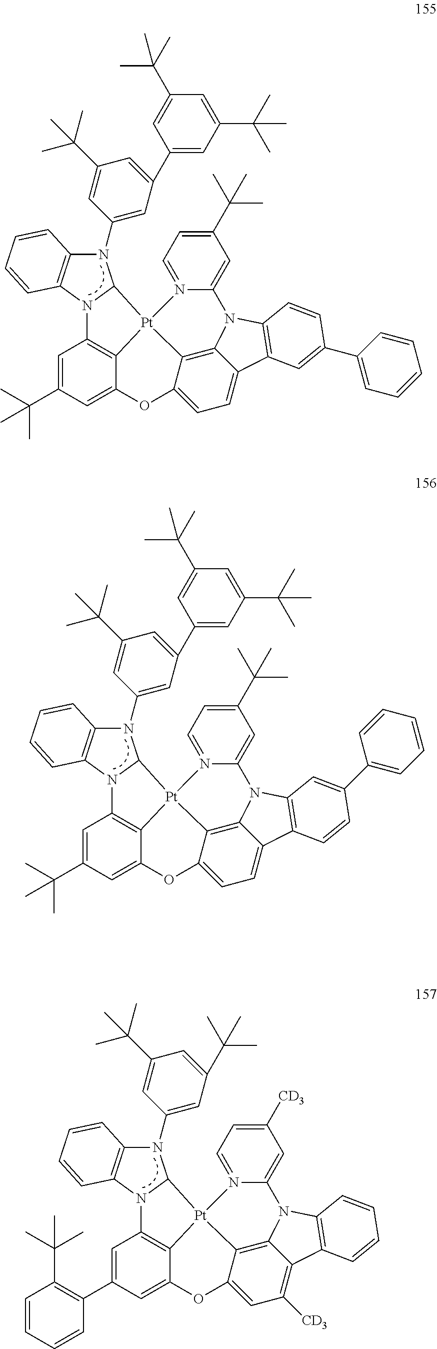

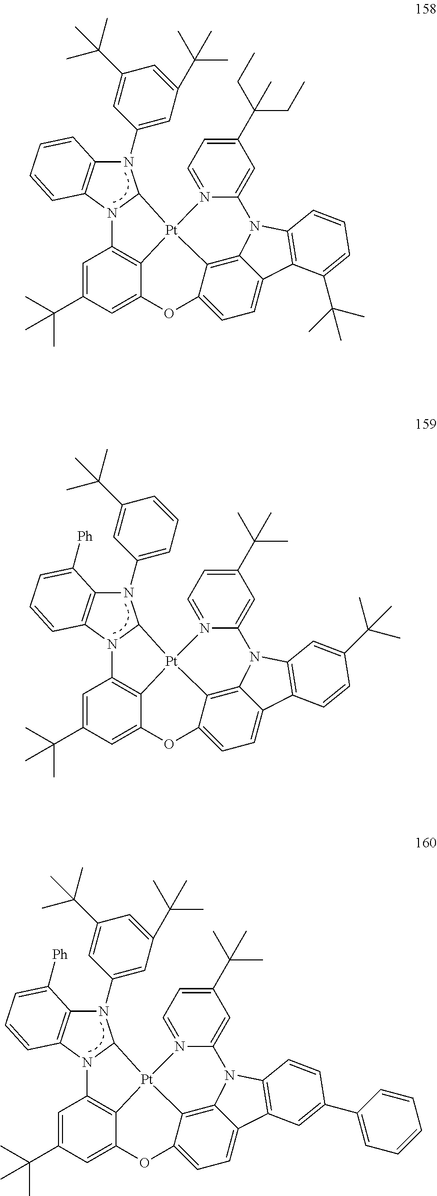

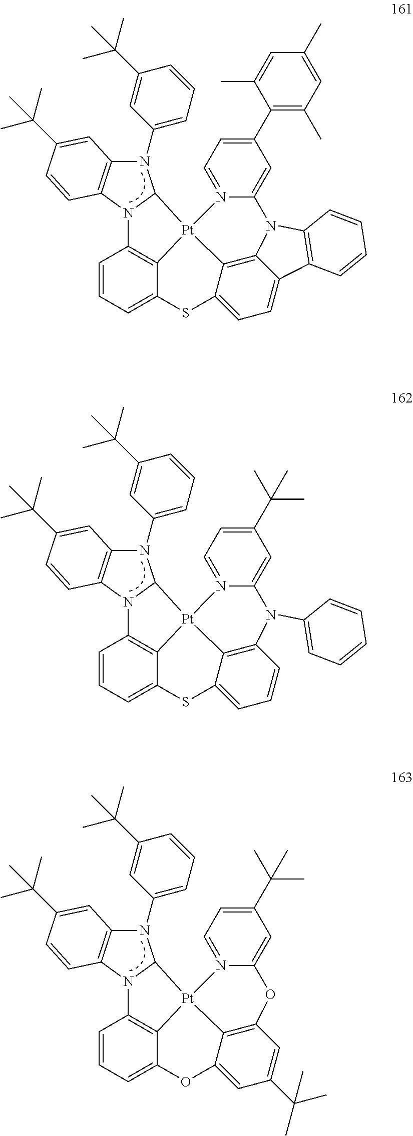

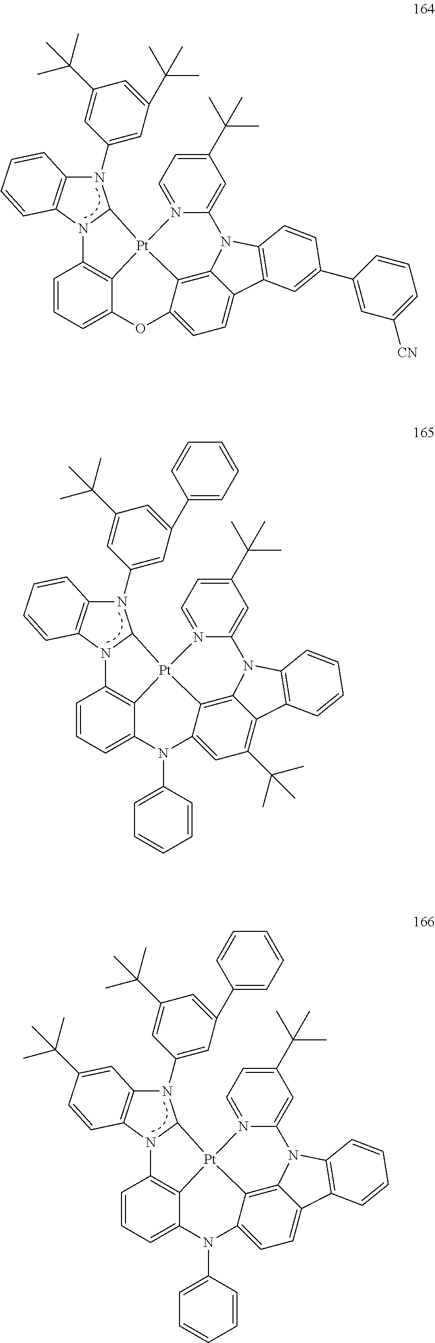

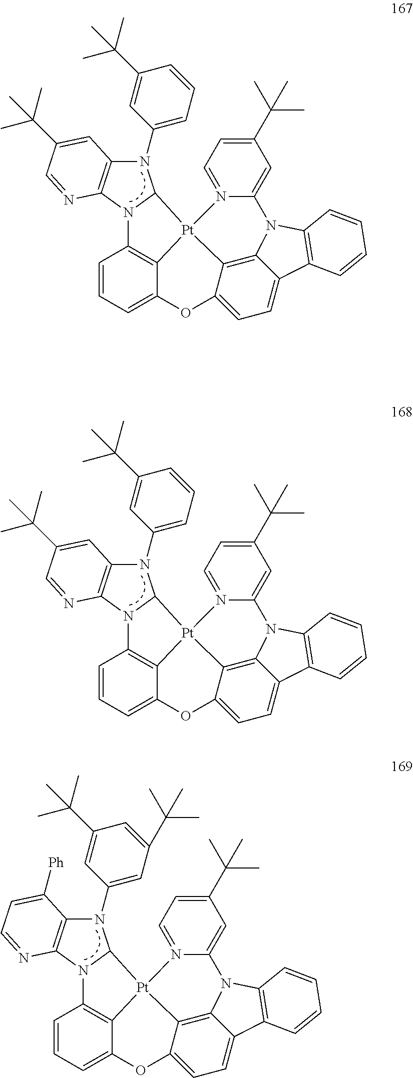

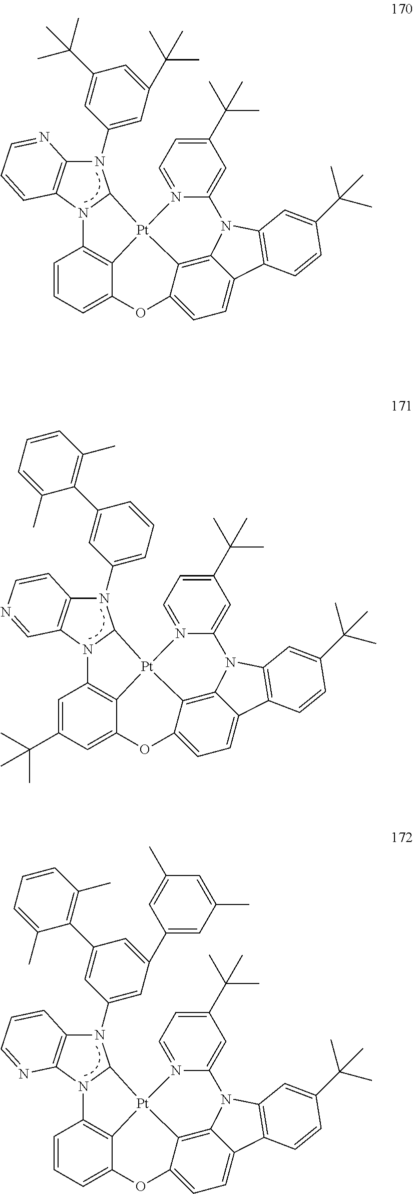

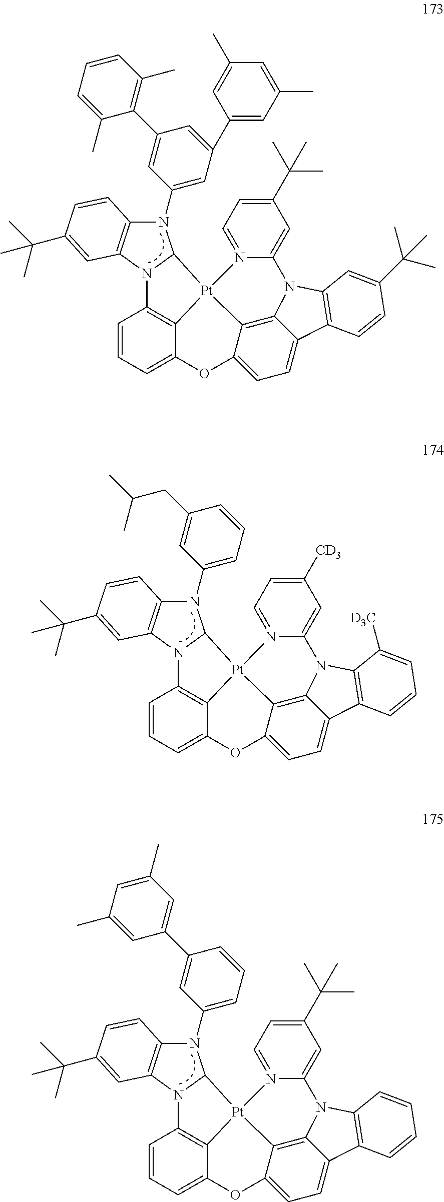

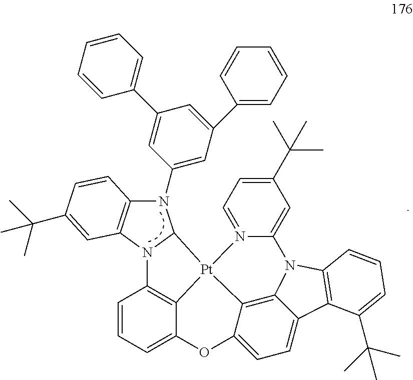

In some embodiments, the sensitizer may be represented by Formula 111 or Formula 112, and in this embodiment, the sensitizer may be referred to as a delayed fluorescence sensitizer:

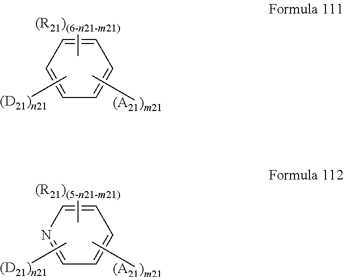

wherein, in Formulae 111 and 112,

A₂₁ may be an acceptor,

D₂₁ may be a donor,

m21 may be 1, 2, or 3, and n21 may b 1, 2, or 3,

in Formula 111, a sum of n21 and m21 may be 6 or less, and in Formula 112, a sum of n21 and m21 may be 5 or less, and

R₂₁ may be hydrogen, deuterium, —F, —Cl, —Br, —I, —SF₅, a hydroxyl group, a cyano group, a nitro group, an amidino group, a hydrazino group, a hydrazono group, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₇-C₆₀ alkyl aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted C₂-C₆₀ alkyl heteroaryl group, a substituted or unsubstituted C₁-C₆₀hetero aryloxy group, a substituted or unsubstituted C₁-C₆₀hetero arylthio group, a substituted or unsubstituted monovalent non-aromatic condensed polycyclic group, a substituted or unsubstituted monovalent non-aromatic condensed heteropolycyclic group, —Si(Q₁)(Q₂)(Q₃), —Ge(Q₁)(Q₂)(Q₃), —C(Q₁)(Q₂)(Q₃), —B(Q₁)(Q₂), —N(Q₁)(Q₂), —P(Q₁)(Q₂), —C(═O)(Q₁), —S(═O)(Q₁), —S(═O)₂(Q₁), —P(═O)(Q₁)(Q₂), or —P(═S)(Q₁)(Q₂), and a plurality of R₂₁(s) may optionally be bound to each other to form a substituted or unsubstituted C₅-C₃₀ carbocyclic group or a substituted or unsubstituted C₁-C₃₀ heterocyclic group,

wherein Q₁ to Q₃ may each independently be hydrogen, deuterium, —F, —Cl, —Br, —I, a hydroxyl group, a cyano group, a nitro group, an amidino group, a hydrazino group, a hydrazono group, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₇-C₆₀ alkyl aryl group, a C₆-C₆₀ aryloxy group, a C₆-C₆₀ arylthio group, a C₁-C₆₀ heteroaryl group, a C₂-C₆₀ alkyl heteroaryl group, a C₁-C₆₀ heteroaryloxy group, a C₁-C₆₀ heteroarylthio group, a monovalent aromatic condensed polycyclic group, a monovalent aromatic condensed heteropolycyclic group, a monovalent non-aromatic condensed polycyclic group, a monovalent non-aromatic condensed heteropolycyclic group, a C₁-C₆₀ alkyl group substituted with at least one deuterium, —F, a cyano group, a C₁-C₆₀ alkyl group, a C₆-C₆₀ aryl group, or any combination thereof, or a C₆-C₆₀ aryl group substituted with at least one deuterium, —F, a cyano group, a C₁-C₆₀ alkyl group, a C₆-C₆₀ aryl group, or any combination thereof.

For example, in Formulae 111 and 112, A₂₁ may be a substituted or unsubstituted π electron-depleted nitrogen-free cyclic group.

In some embodiments, the π electron-depleted nitrogen-free cyclic group may be a benzene group, a heptalene group, an indene group, a naphthalene group, an azulene group, an indacene group, an acenaphthylene group, a fluorene group, a spiro-bifluorene group, a benzofluorene group, a dibenzofluorene group, a phenalene group, a phenanthrene group, an anthracene group, a fluoranthene group, a triphenylene group, a pyrene group, a chrysene group, a naphthacene group, a picene group, a perylene group, a pentacene group, a hexacene group, a pentacene group, a rubicene group, a coronene group, an ovalene group, a pyrrole group, an isoindole group, an indole group, a furan group, a thiophene group, a benzofuran group, a benzothiophene group, a benzocarbazole group, a dibenzocarbazole group, a dibenzofuran group, a dibenzothiophene group, a dibenzothiophene sulfone group, a carbazole group, a dibenzosilole group, an indenocarbazole group, an indolocarbazole group, a benzofurocarbazole group, a benzothienocarbazole group, a triindolobenzene group; or a condensed ring of at least two π electron-depleted nitrogen-free cyclic groups, but embodiments are not limited thereto.

In some embodiments, in Formulae 111 and 112, D₂₁ may be: —F, a cyano group, or a π electron-depleted nitrogen-containing cyclic group;

a C₁-C₆₀ alkyl group, a π electron-depleted nitrogen-containing cyclic group, or π electron-depleted nitrogen-free cyclic group, each substituted with at least one of —F, a cyano group, or any combination thereof; or

a π electron-depleted nitrogen-containing cyclic group substituted with at least one of deuterium, a C₁-C₆₀ alkyl group, a π electron-depleted nitrogen-containing cyclic group, π electron-depleted nitrogen-free cyclic group, or any combination thereof.

In some embodiments, the π electron-depleted nitrogen-free cyclic group may be understood by referring to the description of the π electron-depleted nitrogen-free cyclic group provided herein.

The π electron-depleted nitrogen-containing cyclic group may be a cyclic group having at least one *—N═*′ moiety. Examples thereof may include: an imidazole group, a pyrazole group, a thiazole group, an isothiazole group, an oxazole group, an isoxazole group, a pyridine group, a pyrazine group, a pyridazine group, a pyrimidine group, an indazole group, a purine group, a quinoline group, an isoquinoline group, a benzoquinoline group, a phthalazine group, a naphthyridine group, a quinoxaline group, a quinazoline group, a cinnoline group, a phenanthridine group, an acridine group, a phenanthroline group, a phenazine group, a benzimidazole group, an isobenzothiazole group, a benzoxazole group, an isobenzoxazole group, a triazole group, a tetrazole group, an oxadiazole group, a triazine group, a thiadiazole group, an imidazopyridine group, an imidazopyrimidine group, an azacarbazole group, and a benzimidazolobenzimidazole group; and a condensed ring of at least two π electron-depleted nitrogen-containing cyclic groups.

In some embodiments, the sensitizer may be Groups VII to XII, but embodiments are not limited thereto:

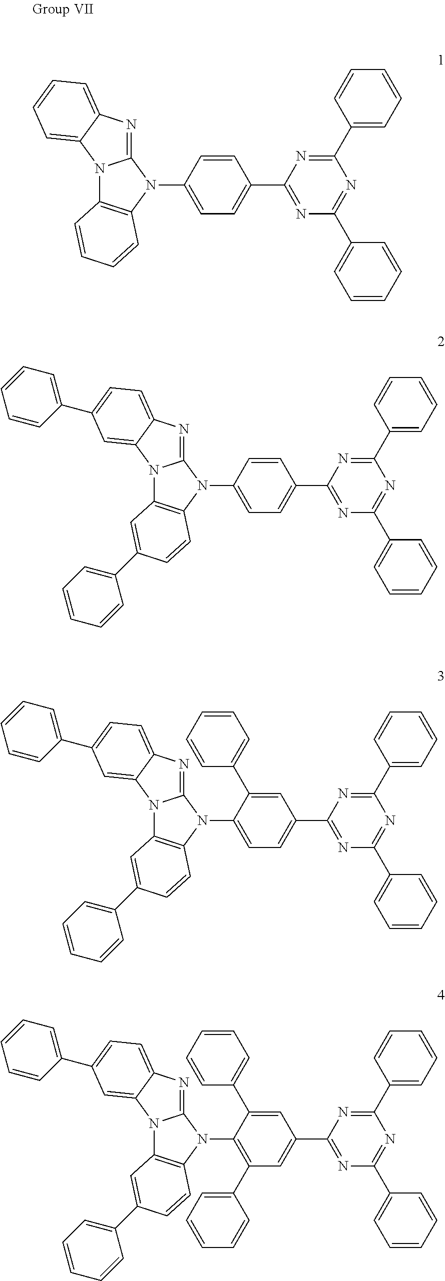 [SMILES: c1ccc(-c2nc(-c3ccccc3)nc(-c3ccc(-n4c5ccccc5n5c6ccccc6nc45)cc3)n2)cc1 ; c1ccc(-c2ccc3nc4n(-c5ccc(-c6nc(-c7ccccc7)nc(-c7ccccc7)n6)cc5)c5ccc(-c6ccccc6)cc5n4c3c2)cc1 ; c1ccc(-c2ccc3nc4n(-c5ccc(-c6nc(-c7ccccc7)nc(-c7ccccc7)n6)cc5-c5ccccc5)c5ccc(-c6ccccc6)cc5n4c3c2)cc1 ; c1ccc(-c2ccc3nc4n(-c5c(-c6ccccc6)cc(-c6nc(-c7ccccc7)nc(-c7ccccc7)n6)cc5-c5ccccc5)c5ccc(-c6ccccc6)cc5n4c3c2)cc1]

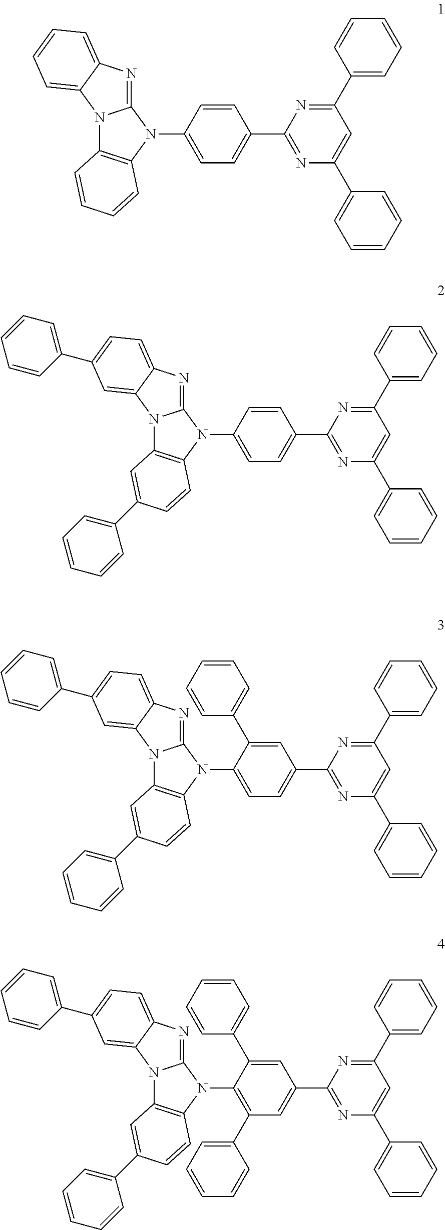 [SMILES: c1ccc(-c2cc(-c3ccccc3)nc(-c3ccc(-n4c5ccccc5n5c6ccccc6nc45)cc3)n2)cc1 ; c1ccc(-c2ccc3nc4n(-c5ccc(-c6nc(-c7ccccc7)cc(-c7ccccc7)n6)cc5)c5ccc(-c6ccccc6)cc5n4c3c2)cc1 ; c1ccc(-c2ccc3nc4n(-c5ccc(-c6nc(-c7ccccc7)cc(-c7ccccc7)n6)cc5-c5ccccc5)c5ccc(-c6ccccc6)cc5n4c3c2)cc1 ; c1ccc(-c2ccc3nc4n(-c5c(-c6ccccc6)cc(-c6nc(-c7ccccc7)cc(-c7ccccc7)n6)cc5-c5ccccc5)c5ccc(-c6ccccc6)cc5n4c3c2)cc1]

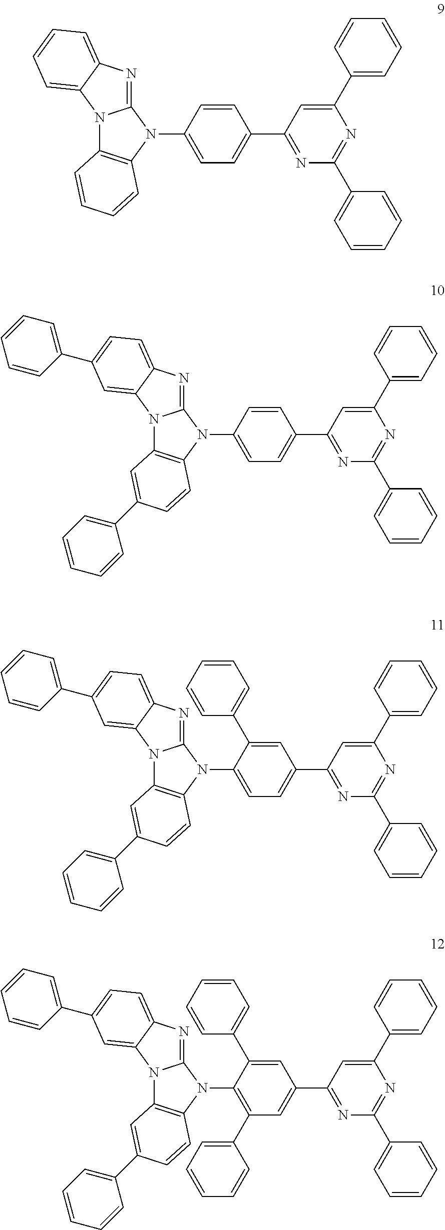 [SMILES: c1ccc(-c2cc(-c3ccc(-n4c5ccccc5n5c6ccccc6nc45)cc3)nc(-c3ccccc3)n2)cc1 ; c1ccc(-c2ccc3nc4n(-c5ccc(-c6cc(-c7ccccc7)nc(-c7ccccc7)n6)cc5)c5ccc(-c6ccccc6)cc5n4c3c2)cc1 ; c1ccc(-c2ccc3nc4n(-c5ccc(-c6cc(-c7ccccc7)nc(-c7ccccc7)n6)cc5-c5ccccc5)c5ccc(-c6ccccc6)cc5n4c3c2)cc1 ; c1ccc(-c2ccc3nc4n(-c5c(-c6ccccc6)cc(-c6cc(-c7ccccc7)nc(-c7ccccc7)n6)cc5-c5ccccc5)c5ccc(-c6ccccc6)cc5n4c3c2)cc1]

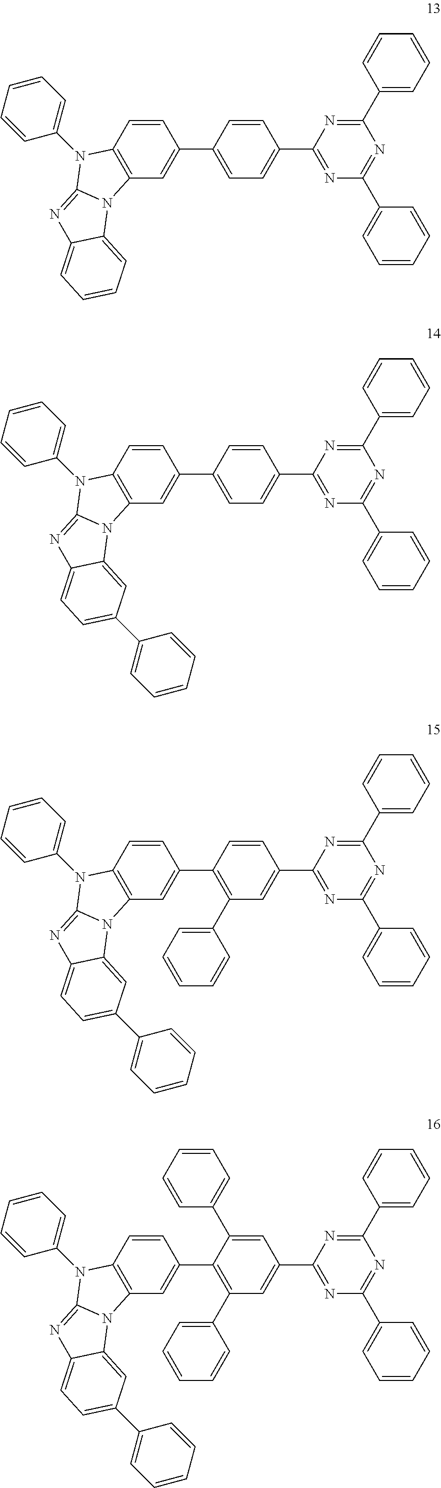 [SMILES: c1ccc(-c2nc(-c3ccccc3)nc(-c3ccc(-c4ccc5c(c4)n4c6ccccc6nc4n5-c4ccccc4)cc3)n2)cc1 ; c1ccc(-c2ccc3nc4n(-c5ccccc5)c5ccc(-c6ccc(-c7nc(-c8ccccc8)nc(-c8ccccc8)n7)cc6)cc5n4c3c2)cc1 ; c1ccc(-c2ccc3nc4n(-c5ccccc5)c5ccc(-c6ccc(-c7nc(-c8ccccc8)nc(-c8ccccc8)n7)cc6-c6ccccc6)cc5n4c3c2)cc1 ; c1ccc(-c2ccc3nc4n(-c5ccccc5)c5ccc(-c6c(-c7ccccc7)cc(-c7nc(-c8ccccc8)nc(-c8ccccc8)n7)cc6-c6ccccc6)cc5n4c3c2)cc1]

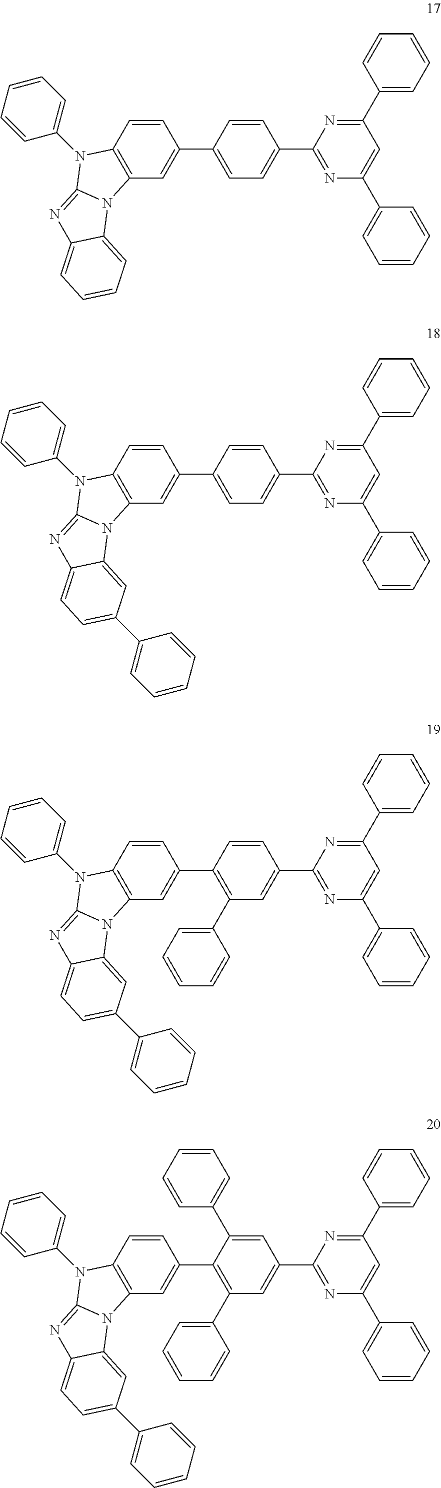 [SMILES: c1ccc(-c2cc(-c3ccccc3)nc(-c3ccc(-c4ccc5c(c4)n4c6ccccc6nc4n5-c4ccccc4)cc3)n2)cc1 ; c1ccc(-c2ccc3nc4n(-c5ccccc5)c5ccc(-c6ccc(-c7nc(-c8ccccc8)cc(-c8ccccc8)n7)cc6)cc5n4c3c2)cc1 ; c1ccc(-c2ccc3nc4n(-c5ccccc5)c5ccc(-c6ccc(-c7nc(-c8ccccc8)cc(-c8ccccc8)n7)cc6-c6ccccc6)cc5n4c3c2)cc1 ; c1ccc(-c2ccc3nc4n(-c5ccccc5)c5ccc(-c6c(-c7ccccc7)cc(-c7nc(-c8ccccc8)cc(-c8ccccc8)n7)cc6-c6ccccc6)cc5n4c3c2)cc1]

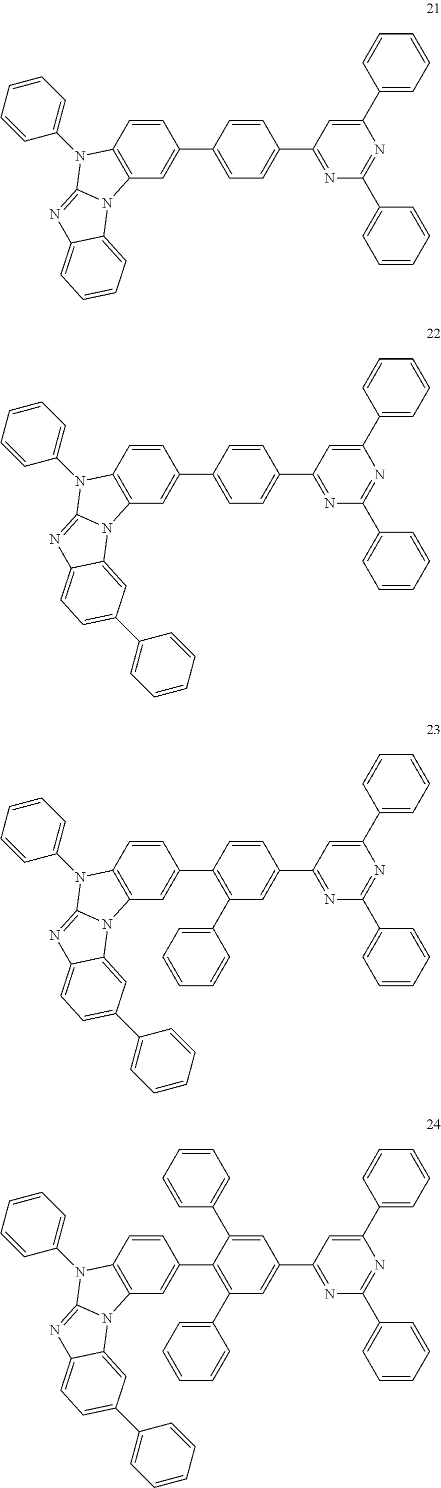 [SMILES: c1ccc(-c2cc(-c3ccc(-c4ccc5c(c4)n4c6ccccc6nc4n5-c4ccccc4)cc3)nc(-c3ccccc3)n2)cc1 ; c1ccc(-c2ccc3nc4n(-c5ccccc5)c5ccc(-c6ccc(-c7cc(-c8ccccc8)nc(-c8ccccc8)n7)cc6)cc5n4c3c2)cc1 ; c1ccc(-c2ccc3nc4n(-c5ccccc5)c5ccc(-c6ccc(-c7cc(-c8ccccc8)nc(-c8ccccc8)n7)cc6-c6ccccc6)cc5n4c3c2)cc1 ; c1ccc(-c2ccc3nc4n(-c5ccccc5)c5ccc(-c6c(-c7ccccc7)cc(-c7cc(-c8ccccc8)nc(-c8ccccc8)n7)cc6-c6ccccc6)cc5n4c3c2)cc1]

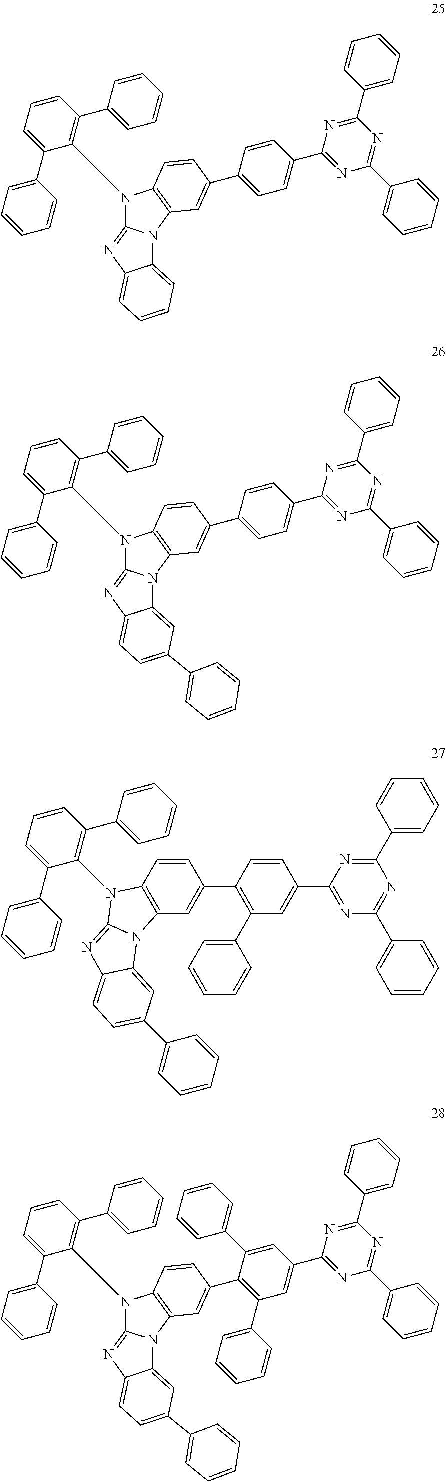 [SMILES: c1ccc(-c2nc(-c3ccccc3)nc(-c3ccc(-c4ccc5c(c4)n4c6ccccc6nc4n5-c4c(-c5ccccc5)cccc4-c4ccccc4)cc3)n2)cc1 ; c1ccc(-c2ccc3nc4n(-c5c(-c6ccccc6)cccc5-c5ccccc5)c5ccc(-c6ccc(-c7nc(-c8ccccc8)nc(-c8ccccc8)n7)cc6)cc5n4c3c2)cc1 ; c1ccc(-c2ccc3nc4n(-c5c(-c6ccccc6)cccc5-c5ccccc5)c5ccc(-c6ccc(-c7nc(-c8ccccc8)nc(-c8ccccc8)n7)cc6-c6ccccc6)cc5n4c3c2)cc1 ; c1ccc(-c2ccc3nc4n(-c5c(-c6ccccc6)cccc5-c5ccccc5)c5ccc(-c6c(-c7ccccc7)cc(-c7nc(-c8ccccc8)nc(-c8ccccc8)n7)cc6-c6ccccc6)cc5n4c3c2)cc1]

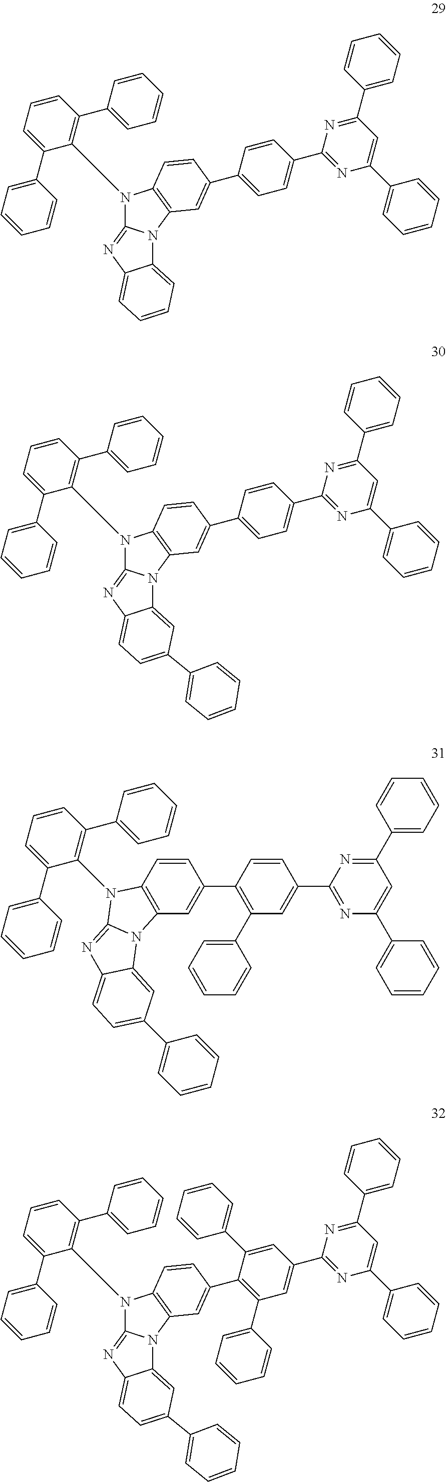 [SMILES: c1ccc(-c2cc(-c3ccccc3)nc(-c3ccc(-c4ccc5c(c4)n4c6ccccc6nc4n5-c4c(-c5ccccc5)cccc4-c4ccccc4)cc3)n2)cc1 ; c1ccc(-c2ccc3nc4n(-c5c(-c6ccccc6)cccc5-c5ccccc5)c5ccc(-c6ccc(-c7nc(-c8ccccc8)cc(-c8ccccc8)n7)cc6)cc5n4c3c2)cc1 ; c1ccc(-c2ccc3nc4n(-c5c(-c6ccccc6)cccc5-c5ccccc5)c5ccc(-c6ccc(-c7nc(-c8ccccc8)cc(-c8ccccc8)n7)cc6-c6ccccc6)cc5n4c3c2)cc1 ; c1ccc(-c2ccc3nc4n(-c5c(-c6ccccc6)cccc5-c5ccccc5)c5ccc(-c6c(-c7ccccc7)cc(-c7nc(-c8ccccc8)cc(-c8ccccc8)n7)cc6-c6ccccc6)cc5n4c3c2)cc1]

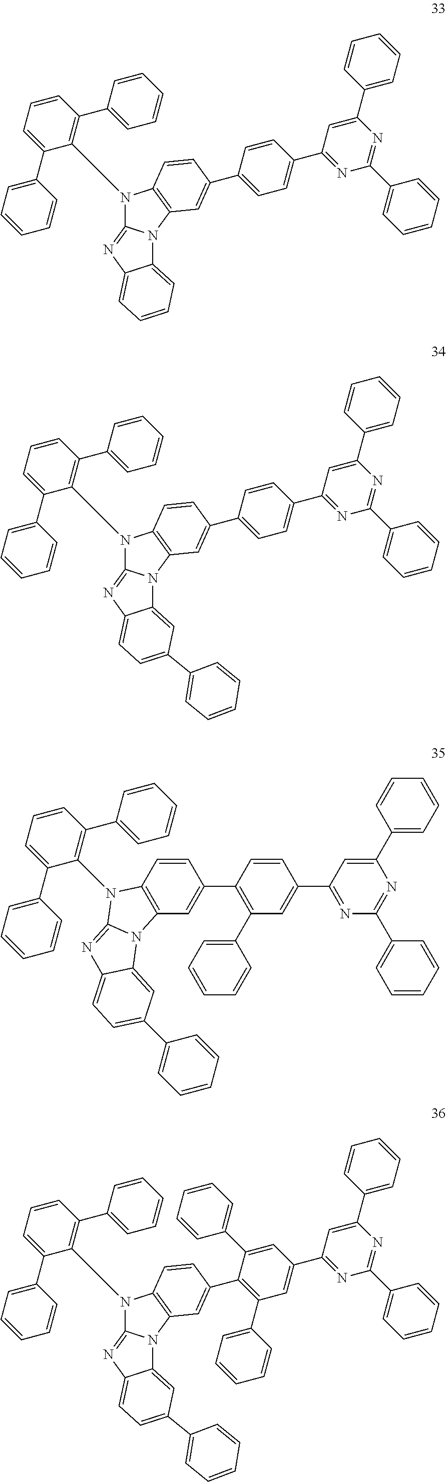 [SMILES: c1ccc(-c2cc(-c3ccc(-c4ccc5c(c4)n4c6ccccc6nc4n5-c4c(-c5ccccc5)cccc4-c4ccccc4)cc3)nc(-c3ccccc3)n2)cc1 ; c1ccc(-c2ccc3nc4n(-c5c(-c6ccccc6)cccc5-c5ccccc5)c5ccc(-c6ccc(-c7cc(-c8ccccc8)nc(-c8ccccc8)n7)cc6)cc5n4c3c2)cc1 ; c1ccc(-c2ccc3nc4n(-c5c(-c6ccccc6)cccc5-c5ccccc5)c5ccc(-c6ccc(-c7cc(-c8ccccc8)nc(-c8ccccc8)n7)cc6-c6ccccc6)cc5n4c3c2)cc1 ; c1ccc(-c2ccc3nc4n(-c5c(-c6ccccc6)cccc5-c5ccccc5)c5ccc(-c6c(-c7ccccc7)cc(-c7cc(-c8ccccc8)nc(-c8ccccc8)n7)cc6-c6ccccc6)cc5n4c3c2)cc1]

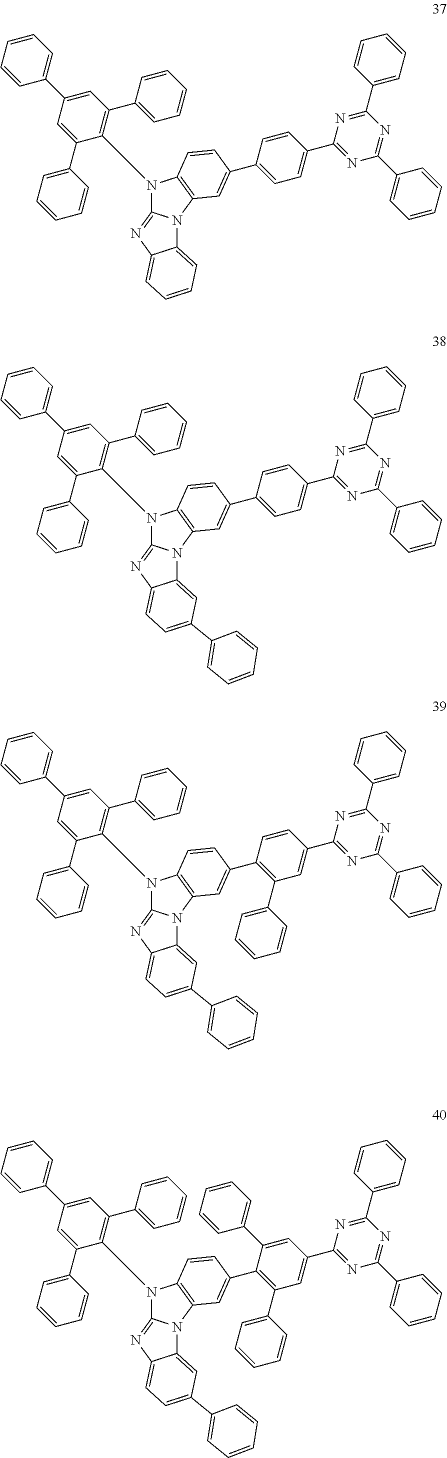

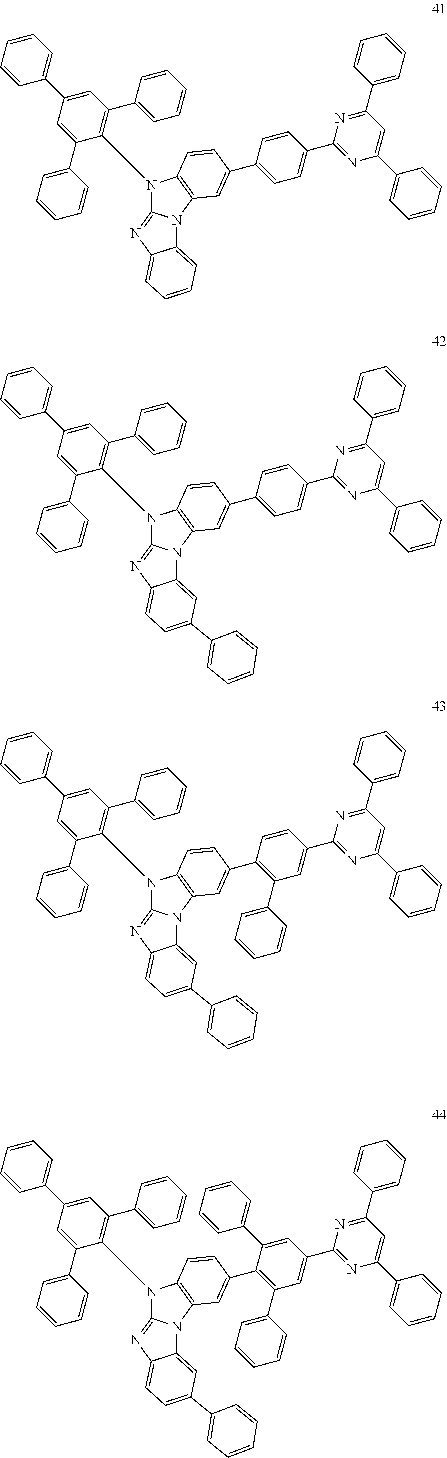

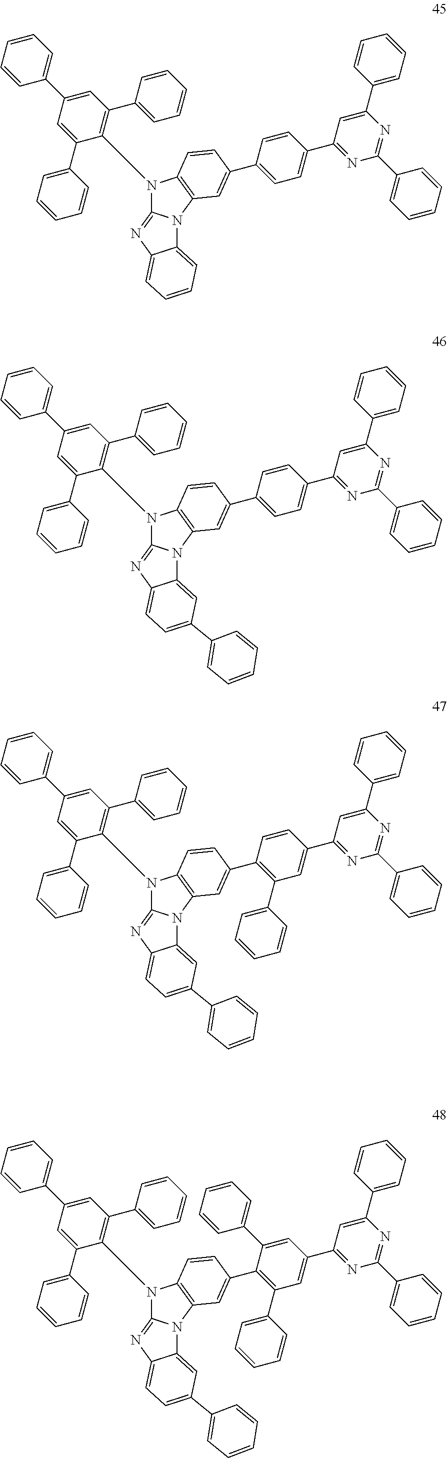

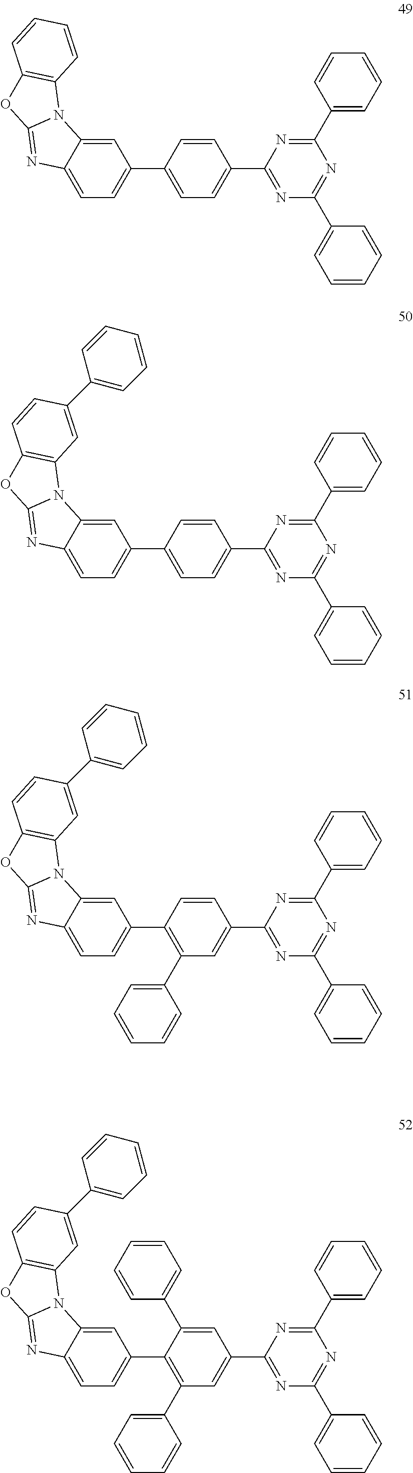 [SMILES: c1ccc(-c2nc(-c3ccccc3)nc(-c3ccc(-c4ccc5nc6oc7ccccc7n6c5c4)cc3)n2)cc1 ; c1ccc(-c2ccc3oc4nc5ccc(-c6ccc(-c7nc(-c8ccccc8)nc(-c8ccccc8)n7)cc6)cc5n4c3c2)cc1 ; c1ccc(-c2ccc3oc4nc5ccc(-c6ccc(-c7nc(-c8ccccc8)nc(-c8ccccc8)n7)cc6-c6ccccc6)cc5n4c3c2)cc1 ; c1ccc(-c2ccc3oc4nc5ccc(-c6c(-c7ccccc7)cc(-c7nc(-c8ccccc8)nc(-c8ccccc8)n7)cc6-c6ccccc6)cc5n4c3c2)cc1]

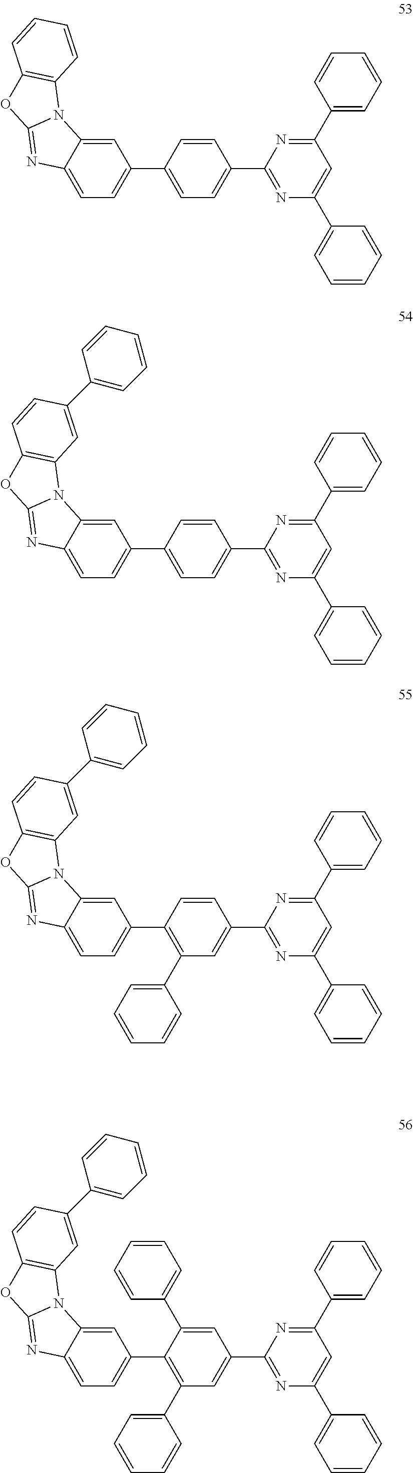 [SMILES: c1ccc(-c2cc(-c3ccccc3)nc(-c3ccc(-c4ccc5nc6oc7ccccc7n6c5c4)cc3)n2)cc1 ; c1ccc(-c2ccc3oc4nc5ccc(-c6ccc(-c7nc(-c8ccccc8)cc(-c8ccccc8)n7)cc6)cc5n4c3c2)cc1 ; c1ccc(-c2ccc3oc4nc5ccc(-c6ccc(-c7nc(-c8ccccc8)cc(-c8ccccc8)n7)cc6-c6ccccc6)cc5n4c3c2)cc1 ; c1ccc(-c2ccc3oc4nc5ccc(-c6c(-c7ccccc7)cc(-c7nc(-c8ccccc8)cc(-c8ccccc8)n7)cc6-c6ccccc6)cc5n4c3c2)cc1]

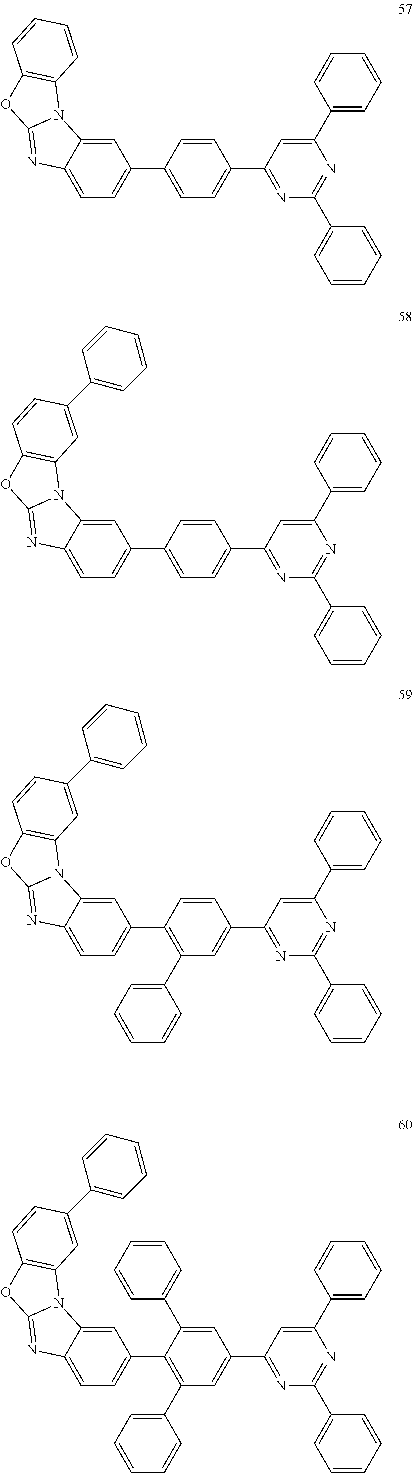 [SMILES: c1ccc(-c2cc(-c3ccc(-c4ccc5nc6oc7ccccc7n6c5c4)cc3)nc(-c3ccccc3)n2)cc1 ; c1ccc(-c2ccc3oc4nc5ccc(-c6ccc(-c7cc(-c8ccccc8)nc(-c8ccccc8)n7)cc6)cc5n4c3c2)cc1 ; c1ccc(-c2ccc3oc4nc5ccc(-c6ccc(-c7cc(-c8ccccc8)nc(-c8ccccc8)n7)cc6-c6ccccc6)cc5n4c3c2)cc1 ; c1ccc(-c2ccc3oc4nc5ccc(-c6c(-c7ccccc7)cc(-c7cc(-c8ccccc8)nc(-c8ccccc8)n7)cc6-c6ccccc6)cc5n4c3c2)cc1]

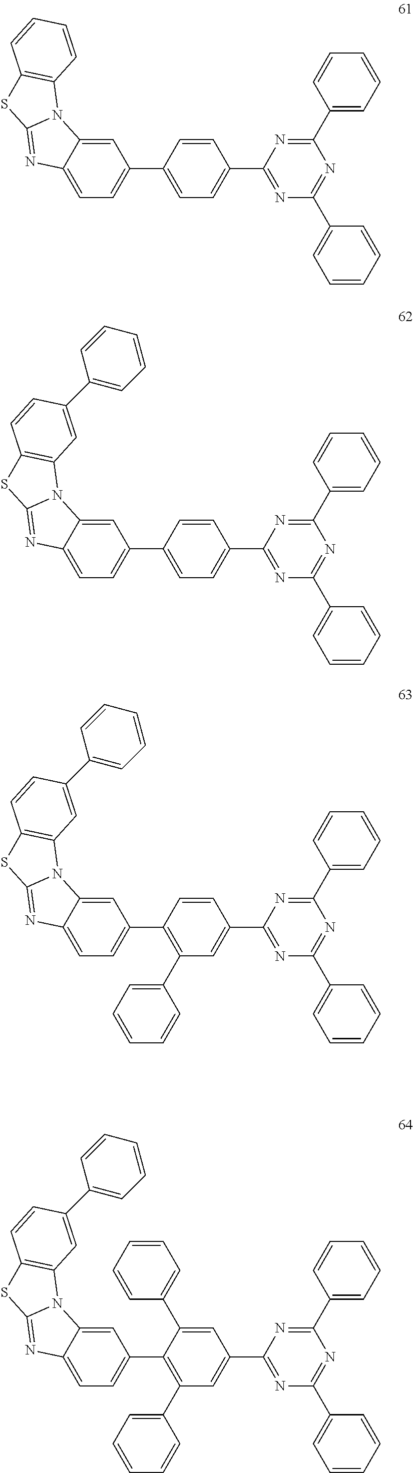 [SMILES: c1ccc(-c2nc(-c3ccccc3)nc(-c3ccc(-c4ccc5nc6sc7ccccc7n6c5c4)cc3)n2)cc1 ; c1ccc(-c2ccc3sc4nc5ccc(-c6ccc(-c7nc(-c8ccccc8)nc(-c8ccccc8)n7)cc6)cc5n4c3c2)cc1 ; c1ccc(-c2ccc3sc4nc5ccc(-c6ccc(-c7nc(-c8ccccc8)nc(-c8ccccc8)n7)cc6-c6ccccc6)cc5n4c3c2)cc1 ; c1ccc(-c2ccc3sc4nc5ccc(-c6c(-c7ccccc7)cc(-c7nc(-c8ccccc8)nc(-c8ccccc8)n7)cc6-c6ccccc6)cc5n4c3c2)cc1]

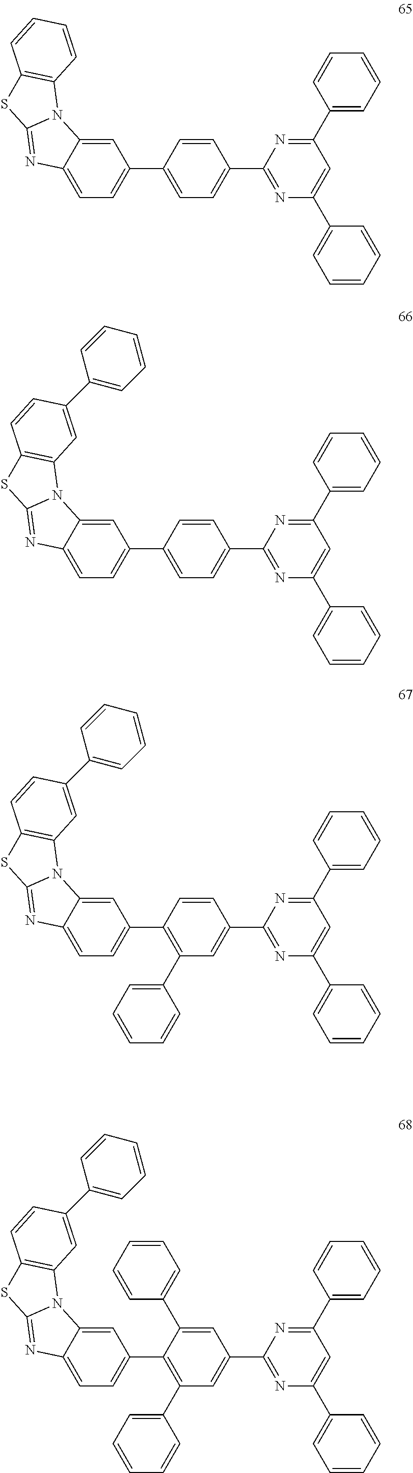 [SMILES: c1ccc(-c2cc(-c3ccccc3)nc(-c3ccc(-c4ccc5nc6sc7ccccc7n6c5c4)cc3)n2)cc1 ; c1ccc(-c2ccc3sc4nc5ccc(-c6ccc(-c7nc(-c8ccccc8)cc(-c8ccccc8)n7)cc6)cc5n4c3c2)cc1 ; c1ccc(-c2ccc3sc4nc5ccc(-c6ccc(-c7nc(-c8ccccc8)cc(-c8ccccc8)n7)cc6-c6ccccc6)cc5n4c3c2)cc1 ; c1ccc(-c2ccc3sc4nc5ccc(-c6c(-c7ccccc7)cc(-c7nc(-c8ccccc8)cc(-c8ccccc8)n7)cc6-c6ccccc6)cc5n4c3c2)cc1]

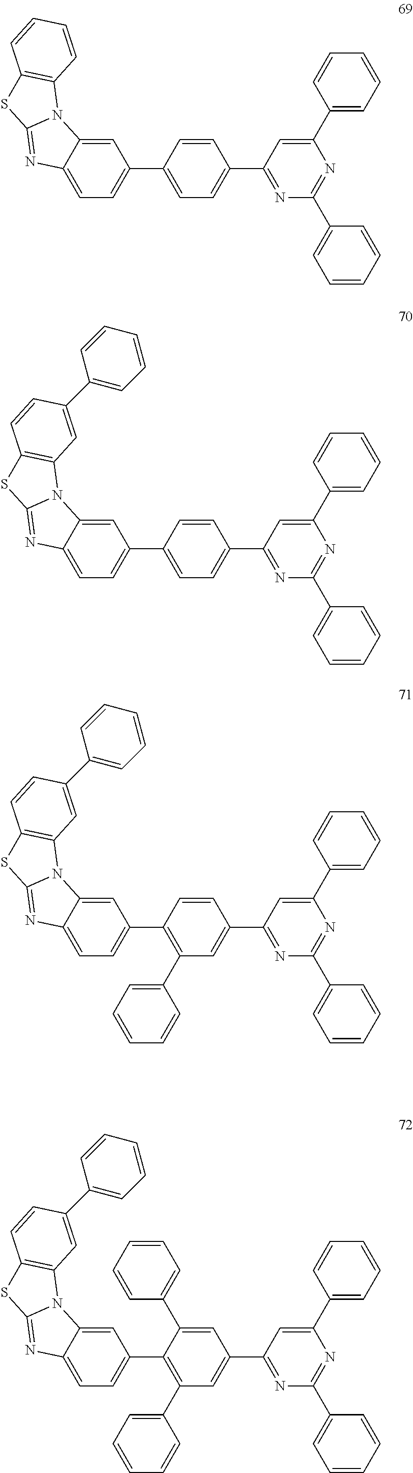 [SMILES: c1ccc(-c2cc(-c3ccc(-c4ccc5nc6sc7ccccc7n6c5c4)cc3)nc(-c3ccccc3)n2)cc1 ; c1ccc(-c2ccc3sc4nc5ccc(-c6ccc(-c7cc(-c8ccccc8)nc(-c8ccccc8)n7)cc6)cc5n4c3c2)cc1 ; c1ccc(-c2ccc3sc4nc5ccc(-c6ccc(-c7cc(-c8ccccc8)nc(-c8ccccc8)n7)cc6-c6ccccc6)cc5n4c3c2)cc1 ; c1ccc(-c2ccc3sc4nc5ccc(-c6c(-c7ccccc7)cc(-c7cc(-c8ccccc8)nc(-c8ccccc8)n7)cc6-c6ccccc6)cc5n4c3c2)cc1]

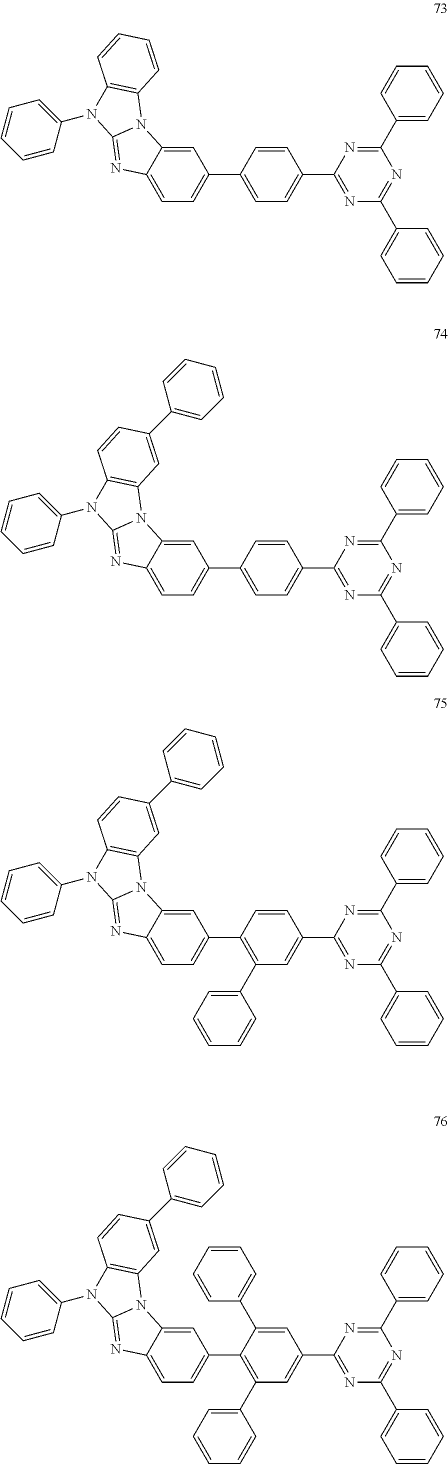 [SMILES: c1ccc(-c2nc(-c3ccccc3)nc(-c3ccc(-c4ccc5nc6n(-c7ccccc7)c7ccccc7n6c5c4)cc3)n2)cc1 ; c1ccc(-c2ccc3c(c2)n2c4cc(-c5ccc(-c6nc(-c7ccccc7)nc(-c7ccccc7)n6)cc5)ccc4nc2n3-c2ccccc2)cc1 ; c1ccc(-c2ccc3c(c2)n2c4cc(-c5ccc(-c6nc(-c7ccccc7)nc(-c7ccccc7)n6)cc5-c5ccccc5)ccc4nc2n3-c2ccccc2)cc1 ; c1ccc(-c2ccc3c(c2)n2c4cc(-c5c(-c6ccccc6)cc(-c6nc(-c7ccccc7)nc(-c7ccccc7)n6)cc5-c5ccccc5)ccc4nc2n3-c2ccccc2)cc1]

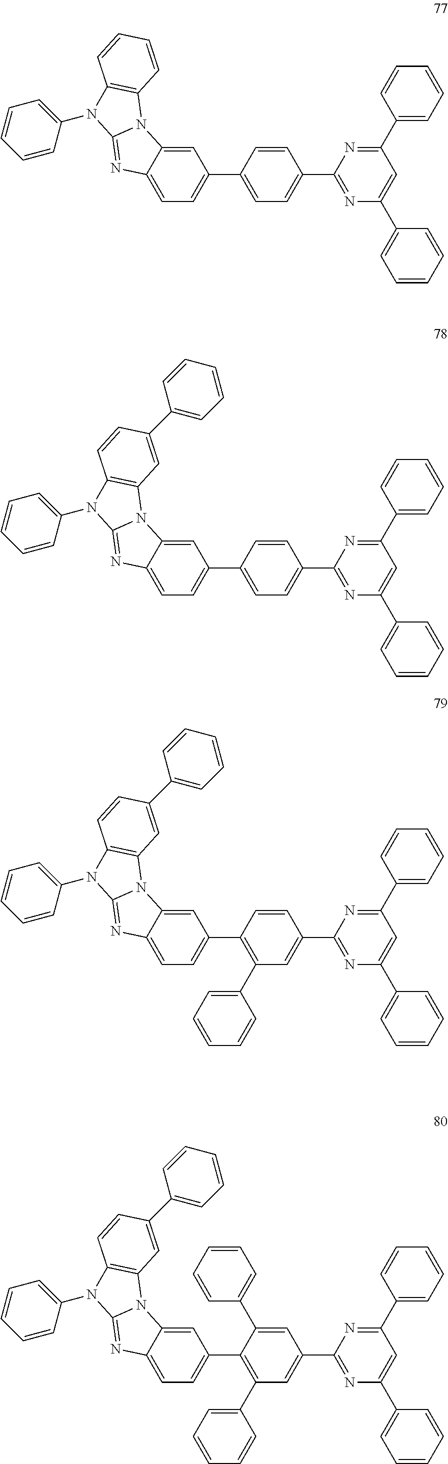 [SMILES: c1ccc(-c2cc(-c3ccccc3)nc(-c3ccc(-c4ccc5nc6n(-c7ccccc7)c7ccccc7n6c5c4)cc3)n2)cc1 ; c1ccc(-c2ccc3c(c2)n2c4cc(-c5ccc(-c6nc(-c7ccccc7)cc(-c7ccccc7)n6)cc5)ccc4nc2n3-c2ccccc2)cc1 ; c1ccc(-c2ccc3c(c2)n2c4cc(-c5ccc(-c6nc(-c7ccccc7)cc(-c7ccccc7)n6)cc5-c5ccccc5)ccc4nc2n3-c2ccccc2)cc1 ; c1ccc(-c2ccc3c(c2)n2c4cc(-c5c(-c6ccccc6)cc(-c6nc(-c7ccccc7)cc(-c7ccccc7)n6)cc5-c5ccccc5)ccc4nc2n3-c2ccccc2)cc1]

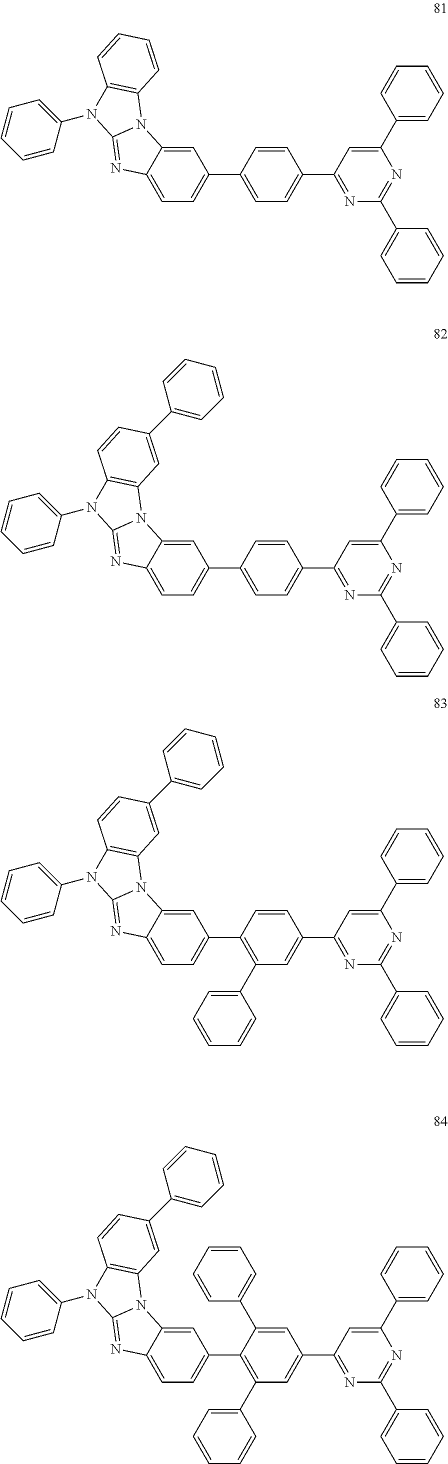 [SMILES: c1ccc(-c2cc(-c3ccc(-c4ccc5nc6n(-c7ccccc7)c7ccccc7n6c5c4)cc3)nc(-c3ccccc3)n2)cc1 ; c1ccc(-c2ccc3c(c2)n2c4cc(-c5ccc(-c6cc(-c7ccccc7)nc(-c7ccccc7)n6)cc5)ccc4nc2n3-c2ccccc2)cc1 ; c1ccc(-c2ccc3c(c2)n2c4cc(-c5ccc(-c6cc(-c7ccccc7)nc(-c7ccccc7)n6)cc5-c5ccccc5)ccc4nc2n3-c2ccccc2)cc1 ; c1ccc(-c2ccc3c(c2)n2c4cc(-c5c(-c6ccccc6)cc(-c6cc(-c7ccccc7)nc(-c7ccccc7)n6)cc5-c5ccccc5)ccc4nc2n3-c2ccccc2)cc1]

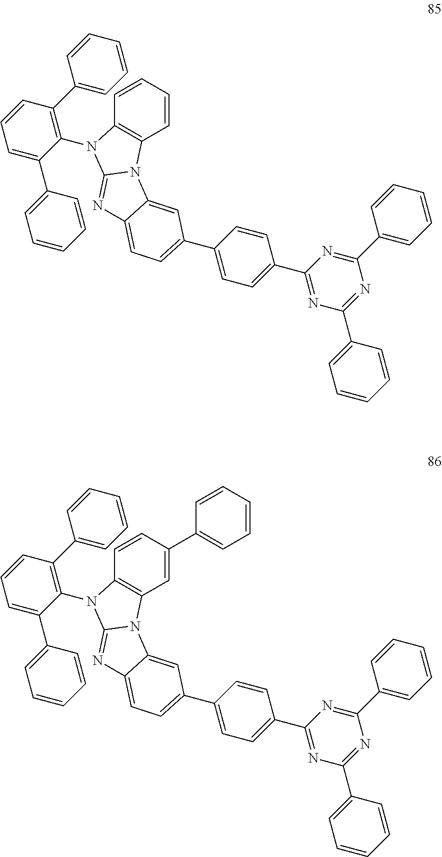 [SMILES: c1ccc(-c2nc(-c3ccccc3)nc(-c3ccc(-c4ccc5nc6n(-c7c(-c8ccccc8)cccc7-c7ccccc7)c7ccccc7n6c5c4)cc3)n2)cc1 ; c1ccc(-c2ccc3c(c2)n2c4cc(-c5ccc(-c6nc(-c7ccccc7)nc(-c7ccccc7)n6)cc5)ccc4nc2n3-c2c(-c3ccccc3)cccc2-c2ccccc2)cc1]

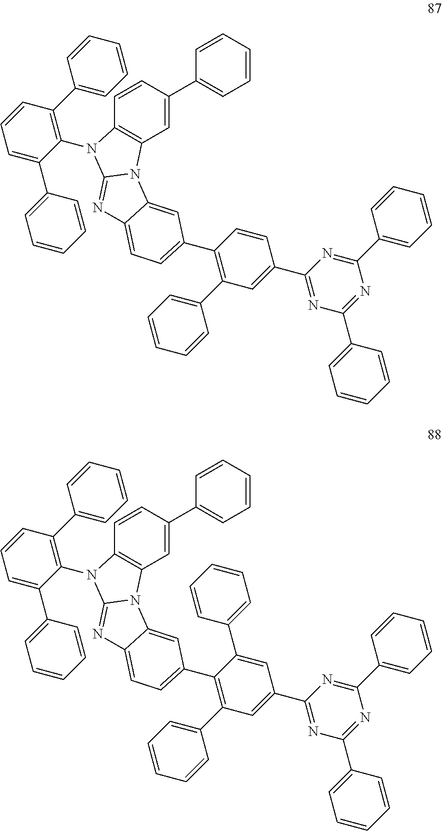 [SMILES: c1ccc(-c2ccc3c(c2)n2c4cc(-c5ccc(-c6nc(-c7ccccc7)nc(-c7ccccc7)n6)cc5-c5ccccc5)ccc4nc2n3-c2c(-c3ccccc3)cccc2-c2ccccc2)cc1 ; c1ccc(-c2ccc3c(c2)n2c4cc(-c5c(-c6ccccc6)cc(-c6nc(-c7ccccc7)nc(-c7ccccc7)n6)cc5-c5ccccc5)ccc4nc2n3-c2c(-c3ccccc3)cccc2-c2ccccc2)cc1]

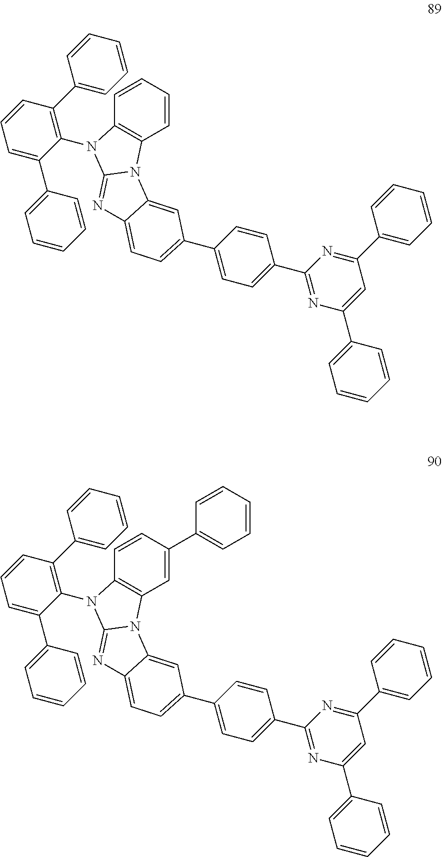 [SMILES: c1ccc(-c2cc(-c3ccccc3)nc(-c3ccc(-c4ccc5nc6n(-c7c(-c8ccccc8)cccc7-c7ccccc7)c7ccccc7n6c5c4)cc3)n2)cc1 ; c1ccc(-c2ccc3c(c2)n2c4cc(-c5ccc(-c6nc(-c7ccccc7)cc(-c7ccccc7)n6)cc5)ccc4nc2n3-c2c(-c3ccccc3)cccc2-c2ccccc2)cc1]

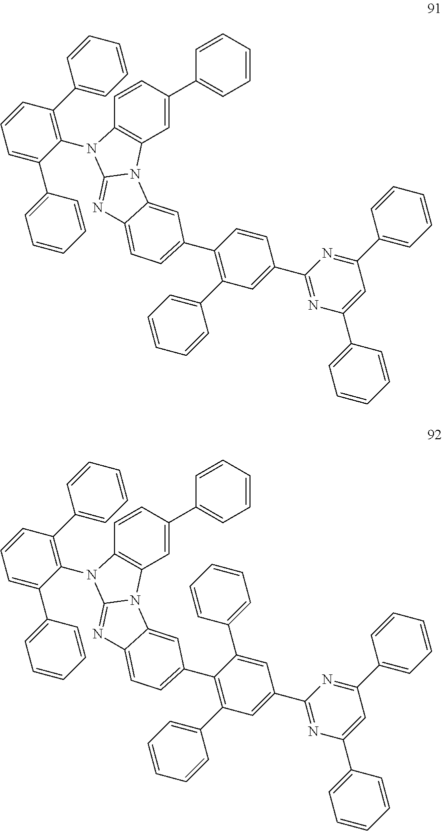 [SMILES: c1ccc(-c2ccc3c(c2)n2c4cc(-c5ccc(-c6nc(-c7ccccc7)cc(-c7ccccc7)n6)cc5-c5ccccc5)ccc4nc2n3-c2c(-c3ccccc3)cccc2-c2ccccc2)cc1 ; c1ccc(-c2ccc3c(c2)n2c4cc(-c5c(-c6ccccc6)cc(-c6nc(-c7ccccc7)cc(-c7ccccc7)n6)cc5-c5ccccc5)ccc4nc2n3-c2c(-c3ccccc3)cccc2-c2ccccc2)cc1]

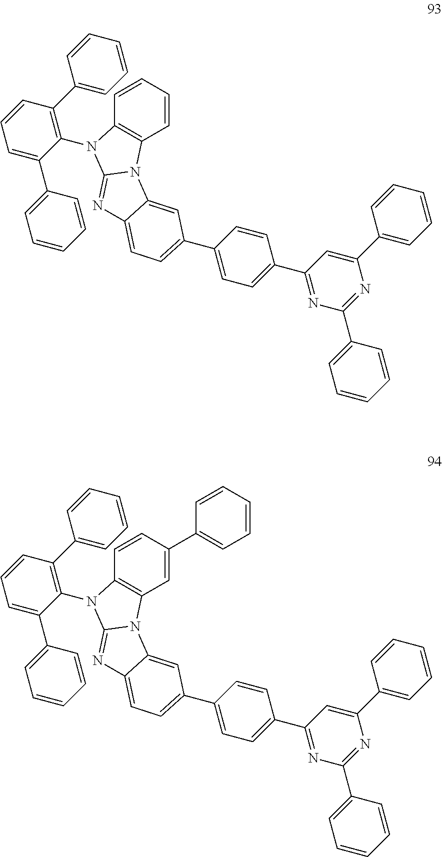 [SMILES: c1ccc(-c2cc(-c3ccc(-c4ccc5nc6n(-c7c(-c8ccccc8)cccc7-c7ccccc7)c7ccccc7n6c5c4)cc3)nc(-c3ccccc3)n2)cc1 ; c1ccc(-c2ccc3c(c2)n2c4cc(-c5ccc(-c6cc(-c7ccccc7)nc(-c7ccccc7)n6)cc5)ccc4nc2n3-c2c(-c3ccccc3)cccc2-c2ccccc2)cc1]

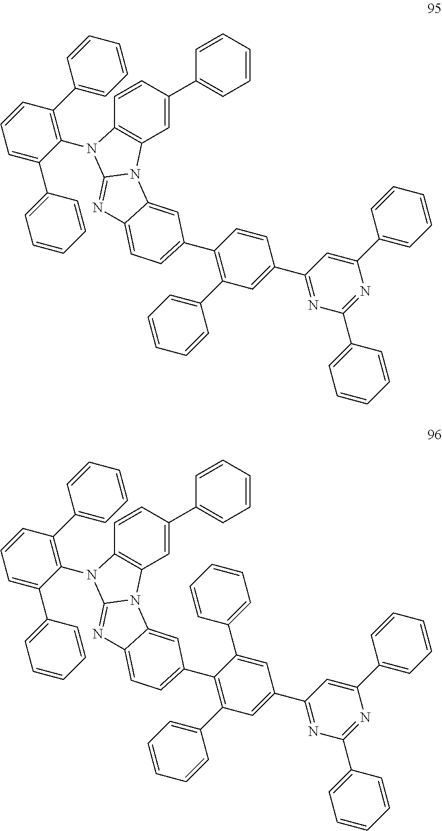 [SMILES: c1ccc(-c2ccc3c(c2)n2c4cc(-c5ccc(-c6cc(-c7ccccc7)nc(-c7ccccc7)n6)cc5-c5ccccc5)ccc4nc2n3-c2c(-c3ccccc3)cccc2-c2ccccc2)cc1 ; c1ccc(-c2ccc3c(c2)n2c4cc(-c5c(-c6ccccc6)cc(-c6cc(-c7ccccc7)nc(-c7ccccc7)n6)cc5-c5ccccc5)ccc4nc2n3-c2c(-c3ccccc3)cccc2-c2ccccc2)cc1]

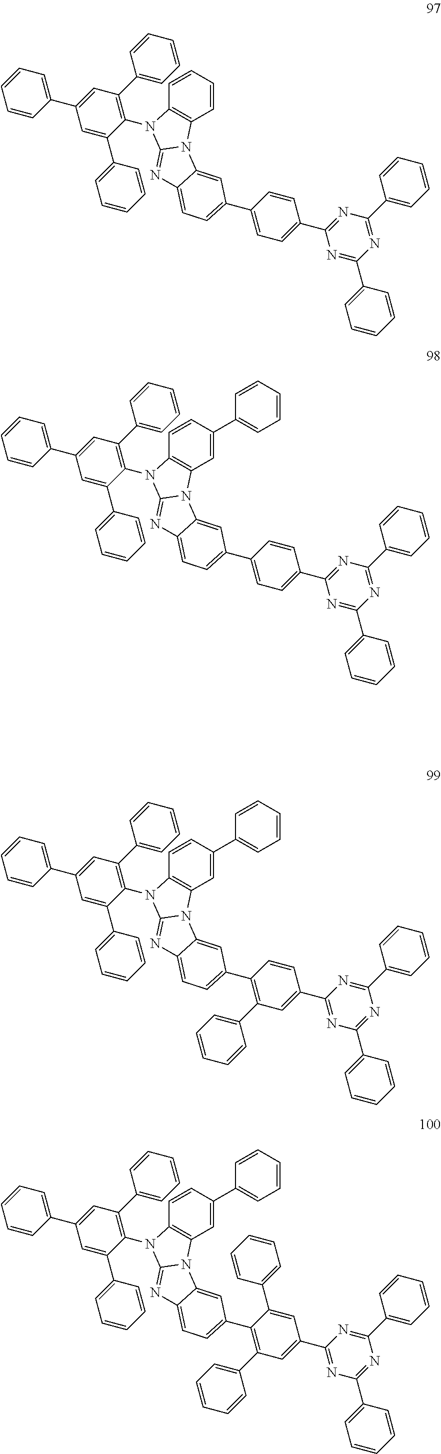

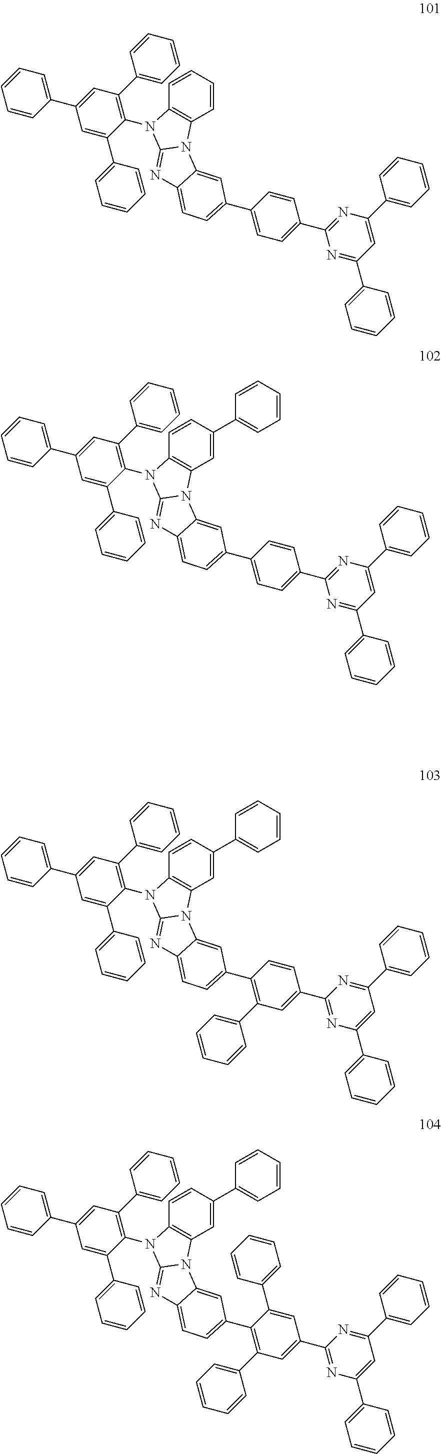

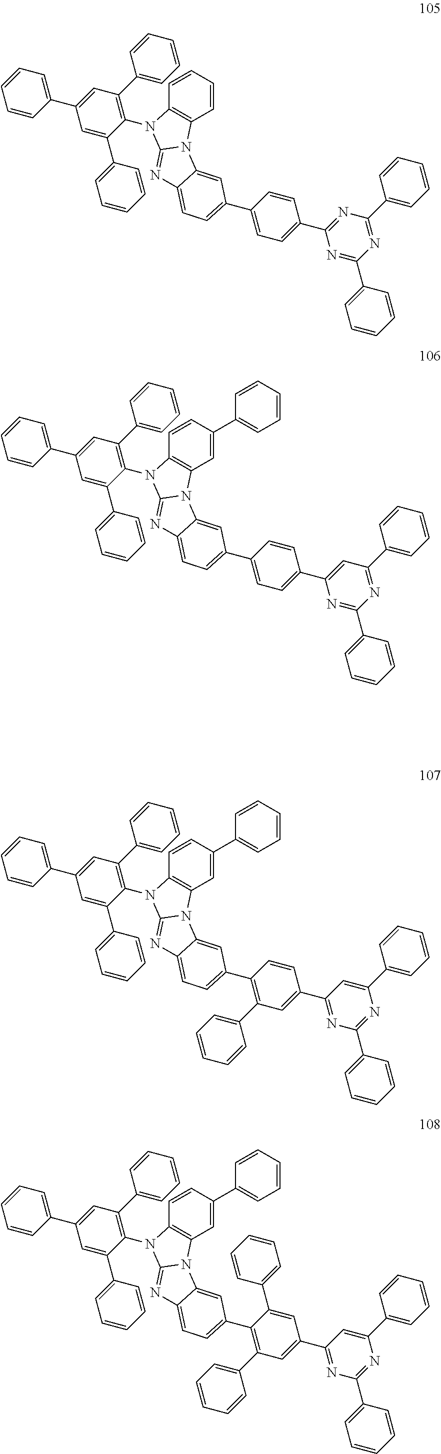

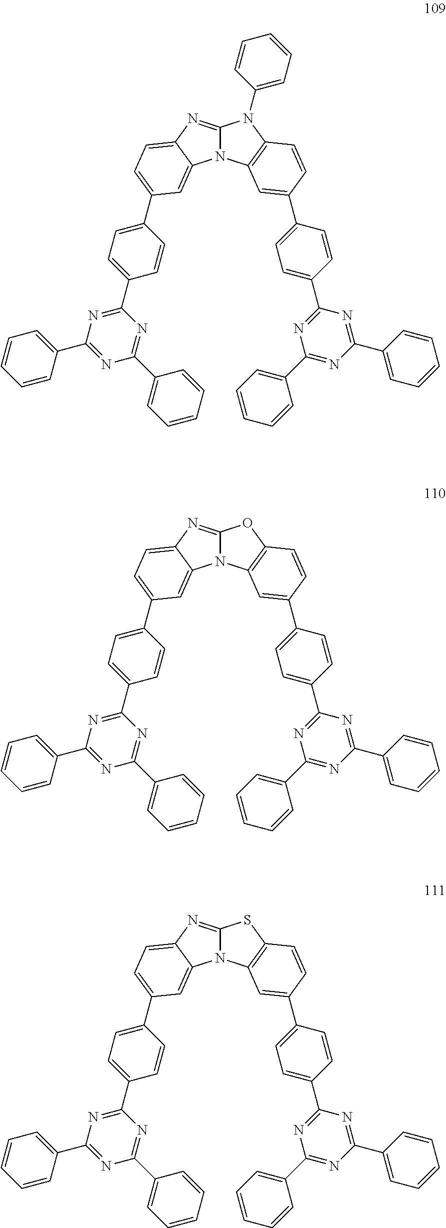 [SMILES: c1ccc(-c2nc(-c3ccccc3)nc(-c3ccc(-c4ccc5nc6n(-c7ccccc7)c7ccc(-c8ccc(-c9nc(-c%10ccccc%10)nc(-c%10ccccc%10)n9)cc8)cc7n6c5c4)cc3)n2)cc1 ; c1ccc(-c2nc(-c3ccccc3)nc(-c3ccc(-c4ccc5nc6oc7ccc(-c8ccc(-c9nc(-c%10ccccc%10)nc(-c%10ccccc%10)n9)cc8)cc7n6c5c4)cc3)n2)cc1 ; c1ccc(-c2nc(-c3ccccc3)nc(-c3ccc(-c4ccc5nc6sc7ccc(-c8ccc(-c9nc(-c%10ccccc%10)nc(-c%10ccccc%10)n9)cc8)cc7n6c5c4)cc3)n2)cc1]

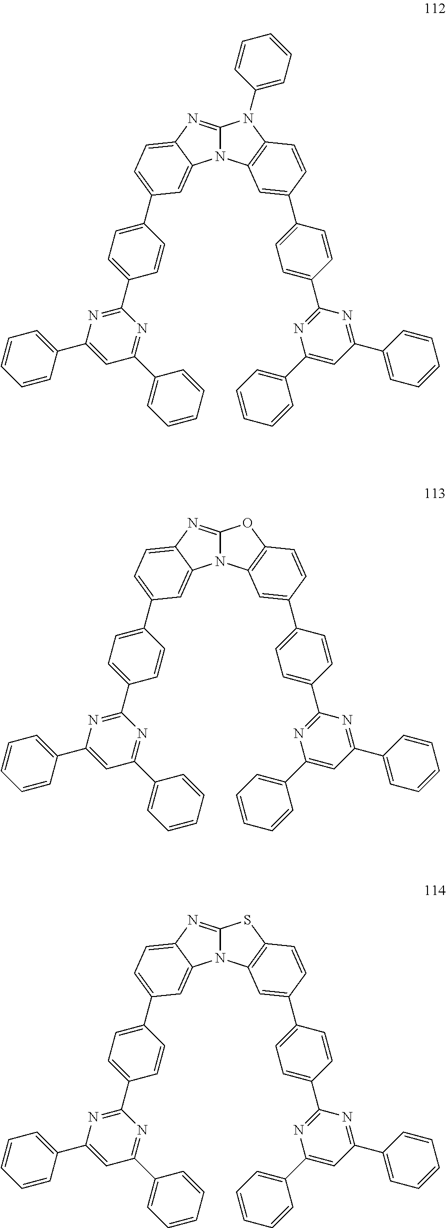 [SMILES: c1ccc(-c2cc(-c3ccccc3)nc(-c3ccc(-c4ccc5nc6n(-c7ccccc7)c7ccc(-c8ccc(-c9nc(-c%10ccccc%10)cc(-c%10ccccc%10)n9)cc8)cc7n6c5c4)cc3)n2)cc1 ; c1ccc(-c2cc(-c3ccccc3)nc(-c3ccc(-c4ccc5nc6oc7ccc(-c8ccc(-c9nc(-c%10ccccc%10)cc(-c%10ccccc%10)n9)cc8)cc7n6c5c4)cc3)n2)cc1 ; c1ccc(-c2cc(-c3ccccc3)nc(-c3ccc(-c4ccc5nc6sc7ccc(-c8ccc(-c9nc(-c%10ccccc%10)cc(-c%10ccccc%10)n9)cc8)cc7n6c5c4)cc3)n2)cc1]

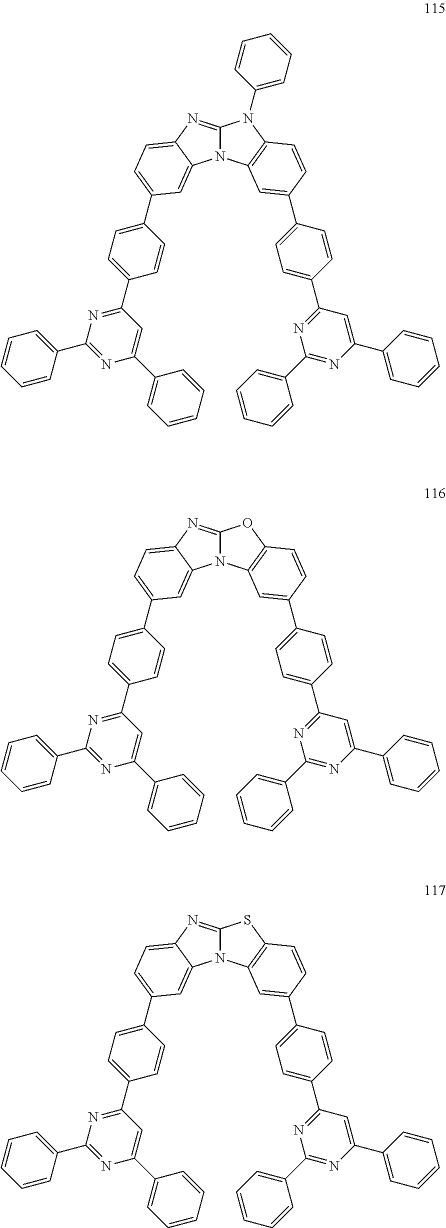 [SMILES: c1ccc(-c2cc(-c3ccc(-c4ccc5nc6n(-c7ccccc7)c7ccc(-c8ccc(-c9cc(-c%10ccccc%10)nc(-c%10ccccc%10)n9)cc8)cc7n6c5c4)cc3)nc(-c3ccccc3)n2)cc1 ; c1ccc(-c2cc(-c3ccc(-c4ccc5nc6oc7ccc(-c8ccc(-c9cc(-c%10ccccc%10)nc(-c%10ccccc%10)n9)cc8)cc7n6c5c4)cc3)nc(-c3ccccc3)n2)cc1 ; c1ccc(-c2cc(-c3ccc(-c4ccc5nc6sc7ccc(-c8ccc(-c9cc(-c%10ccccc%10)nc(-c%10ccccc%10)n9)cc8)cc7n6c5c4)cc3)nc(-c3ccccc3)n2)cc1]

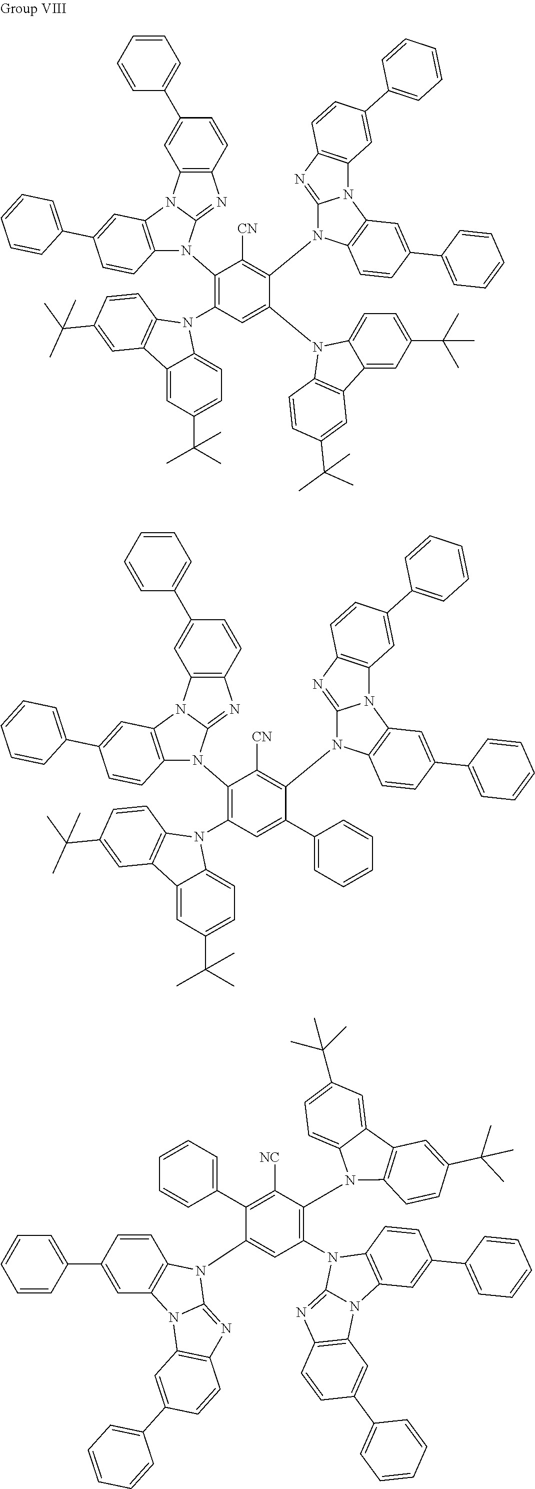

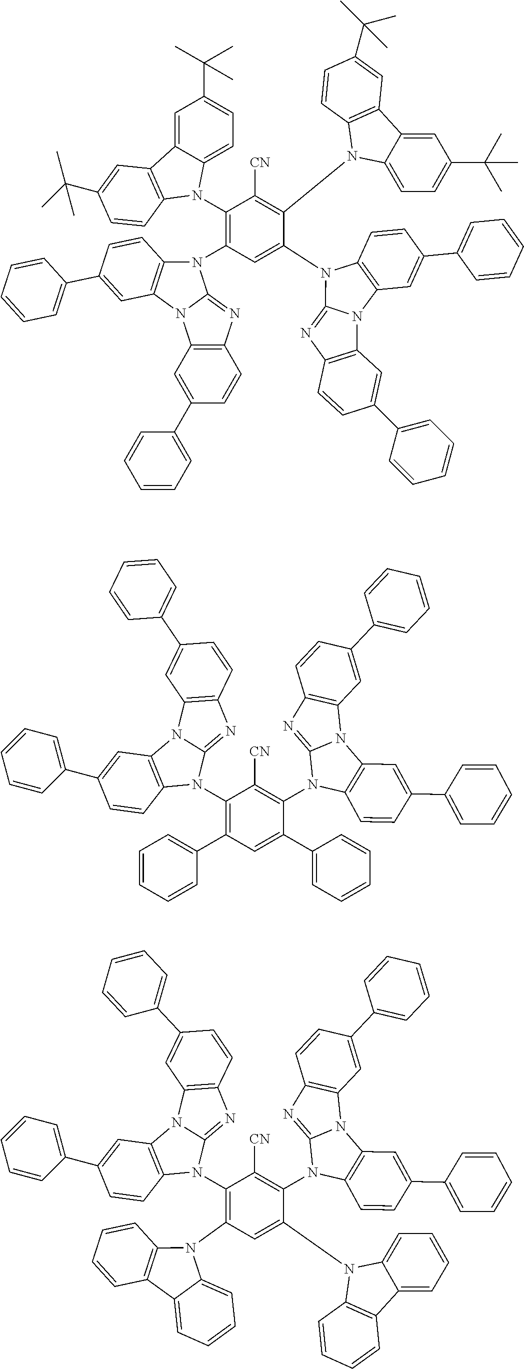 [SMILES: CC(C)(C)c1ccc2c(c1)c1cc(C(C)(C)C)ccc1n2-c1c(-n2c3ccc(-c4ccccc4)cc3n3c4cc(-c5ccccc5)ccc4nc23)cc(-n2c3ccc(-c4ccccc4)cc3n3c4cc(-c5ccccc5)ccc4nc23)c(-n2c3ccc(C(C)(C)C)cc3c3cc(C(C)(C)C)ccc32)c1C#N ; N#Cc1c(-n2c3ccc(-c4ccccc4)cc3n3c4cc(-c5ccccc5)ccc4nc23)c(-c2ccccc2)cc(-c2ccccc2)c1-n1c2ccc(-c3ccccc3)cc2n2c3cc(-c4ccccc4)ccc3nc12 ; N#Cc1c(-n2c3ccc(-c4ccccc4)cc3n3c4cc(-c5ccccc5)ccc4nc23)c(-n2c3ccccc3c3ccccc32)cc(-n2c3ccccc3c3ccccc32)c1-n1c2ccc(-c3ccccc3)cc2n2c3cc(-c4ccccc4)ccc3nc12]

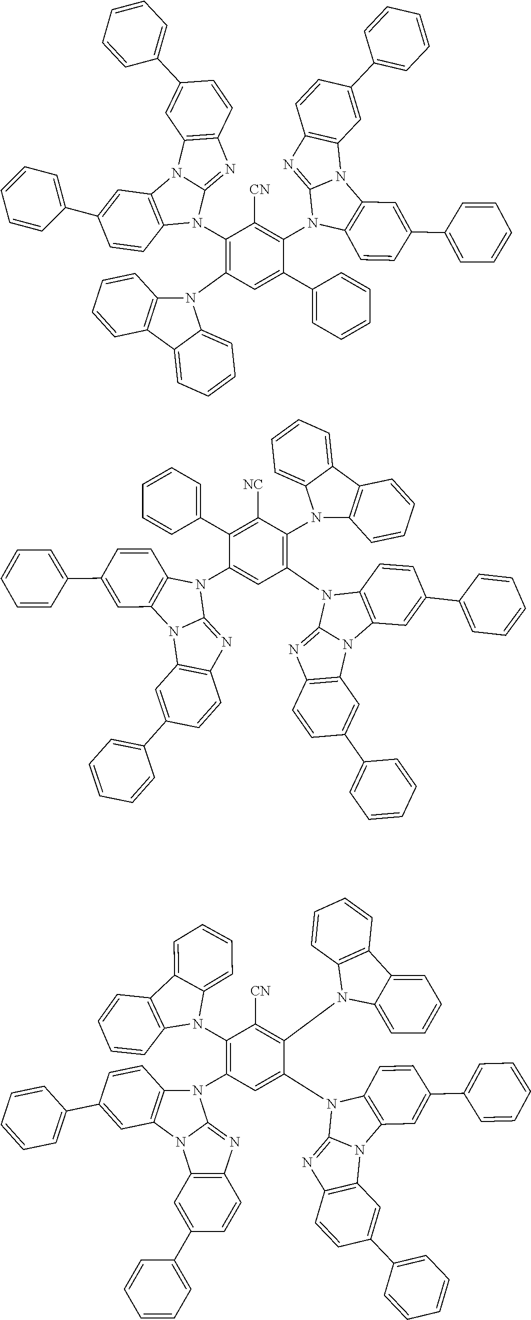 [SMILES: N#Cc1c(-n2c3ccc(-c4ccccc4)cc3n3c4cc(-c5ccccc5)ccc4nc23)c(-c2ccccc2)cc(-n2c3ccccc3c3ccccc32)c1-n1c2ccc(-c3ccccc3)cc2n2c3cc(-c4ccccc4)ccc3nc12 ; N#Cc1c(-c2ccccc2)c(-n2c3ccc(-c4ccccc4)cc3n3c4cc(-c5ccccc5)ccc4nc23)cc(-n2c3ccc(-c4ccccc4)cc3n3c4cc(-c5ccccc5)ccc4nc23)c1-n1c2ccccc2c2ccccc21 ; N#Cc1c(-n2c3ccccc3c3ccccc32)c(-n2c3ccc(-c4ccccc4)cc3n3c4cc(-c5ccccc5)ccc4nc23)cc(-n2c3ccc(-c4ccccc4)cc3n3c4cc(-c5ccccc5)ccc4nc23)c1-n1c2ccccc2c2ccccc21]

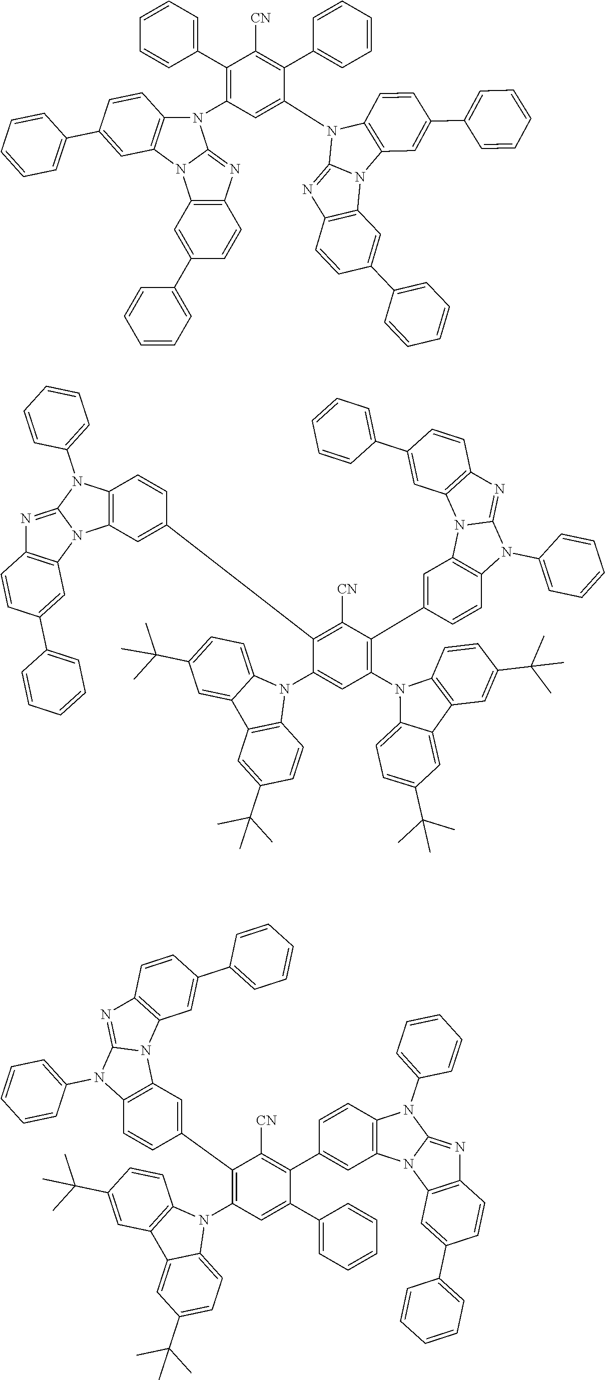 [SMILES: N#Cc1c(-c2ccccc2)c(-n2c3ccc(-c4ccccc4)cc3n3c4cc(-c5ccccc5)ccc4nc23)cc(-n2c3ccc(-c4ccccc4)cc3n3c4cc(-c5ccccc5)ccc4nc23)c1-c1ccccc1 ; CC(C)(C)c1ccc2c(c1)c1cc(C(C)(C)C)ccc1n2-c1cc(-n2c3ccc(C(C)(C)C)cc3c3cc(C(C)(C)C)ccc32)c(-c2ccc3c(c2)n2c4cc(-c5ccccc5)ccc4nc2n3-c2ccccc2)c(C#N)c1-c1ccc2c(c1)n1c3cc(-c4ccccc4)ccc3nc1n2-c1ccccc1 ; CC(C)(C)c1ccc2c(c1)c1cc(C(C)(C)C)ccc1n2-c1cc(-c2ccccc2)c(-c2ccc3c(c2)n2c4cc(-c5ccccc5)ccc4nc2n3-c2ccccc2)c(C#N)c1-c1ccc2c(c1)n1c3cc(-c4ccccc4)ccc3nc1n2-c1ccccc1]

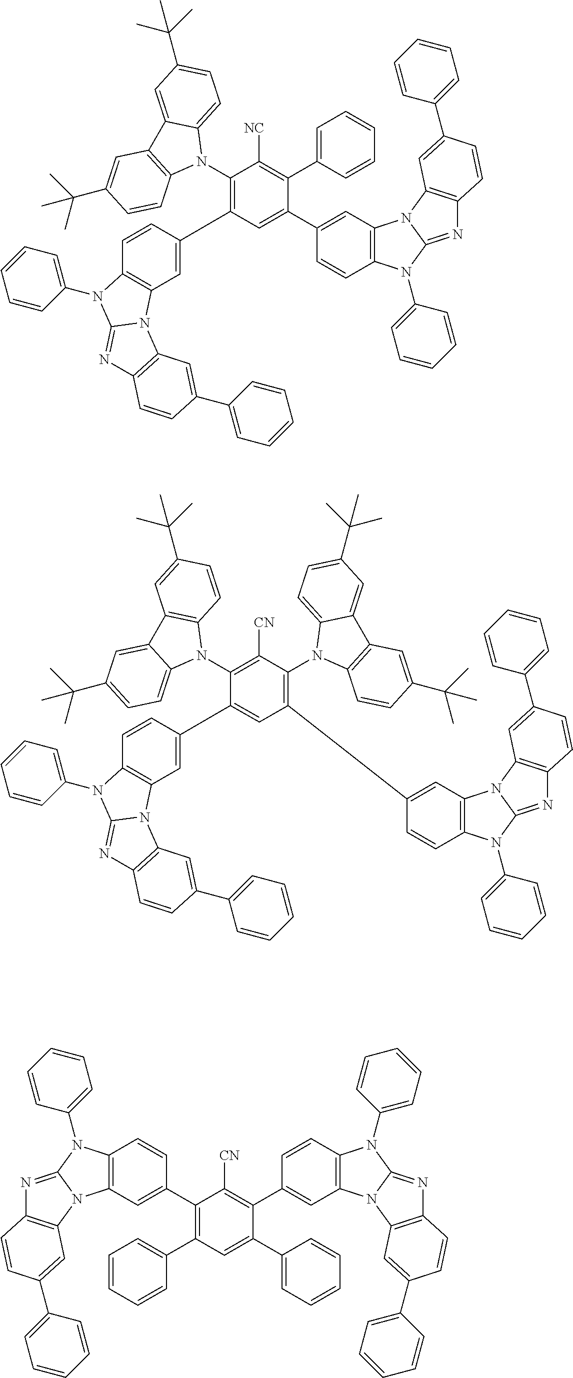

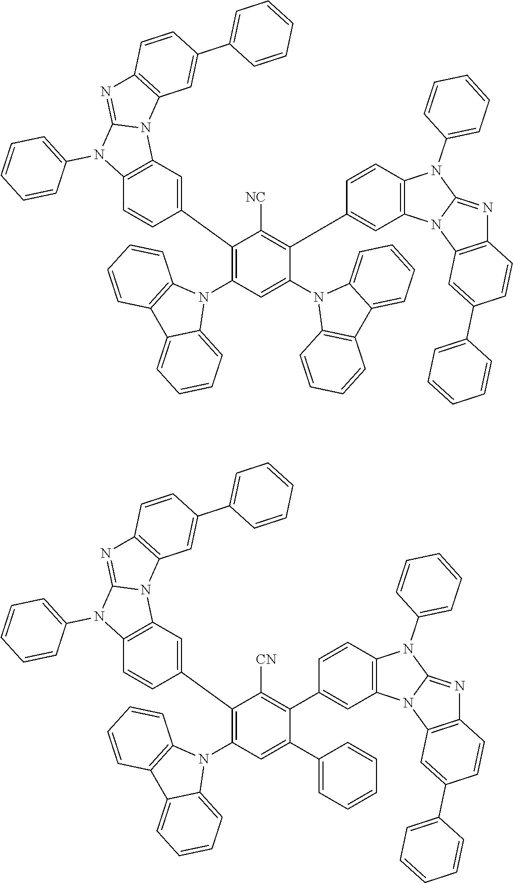 [SMILES: N#Cc1c(-c2ccc3c(c2)n2c4cc(-c5ccccc5)ccc4nc2n3-c2ccccc2)c(-n2c3ccccc3c3ccccc32)cc(-n2c3ccccc3c3ccccc32)c1-c1ccc2c(c1)n1c3cc(-c4ccccc4)ccc3nc1n2-c1ccccc1 ; N#Cc1c(-c2ccc3c(c2)n2c4cc(-c5ccccc5)ccc4nc2n3-c2ccccc2)c(-c2ccccc2)cc(-n2c3ccccc3c3ccccc32)c1-c1ccc2c(c1)n1c3cc(-c4ccccc4)ccc3nc1n2-c1ccccc1]

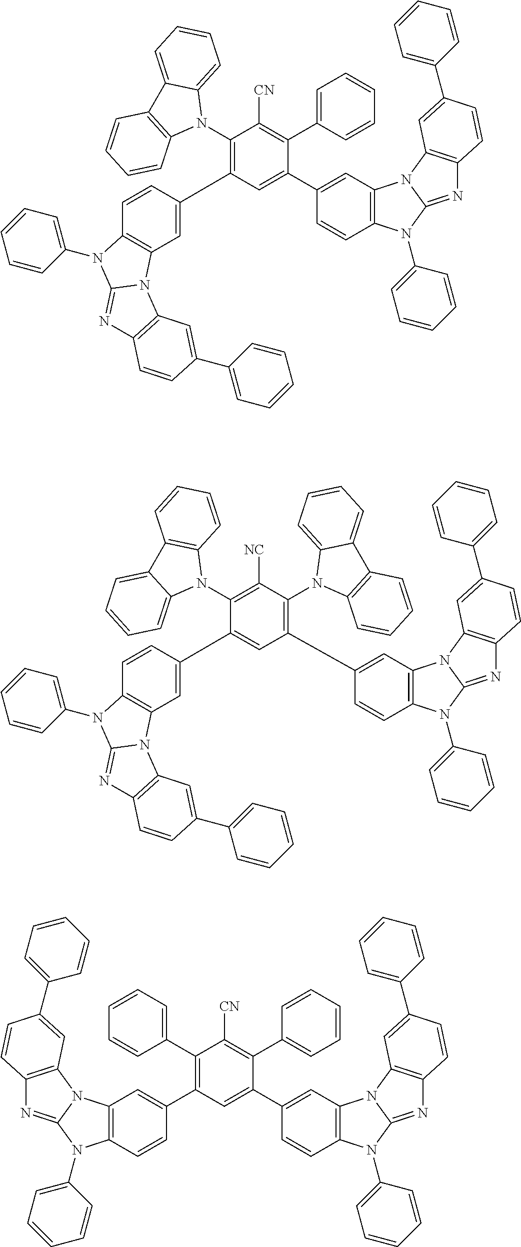

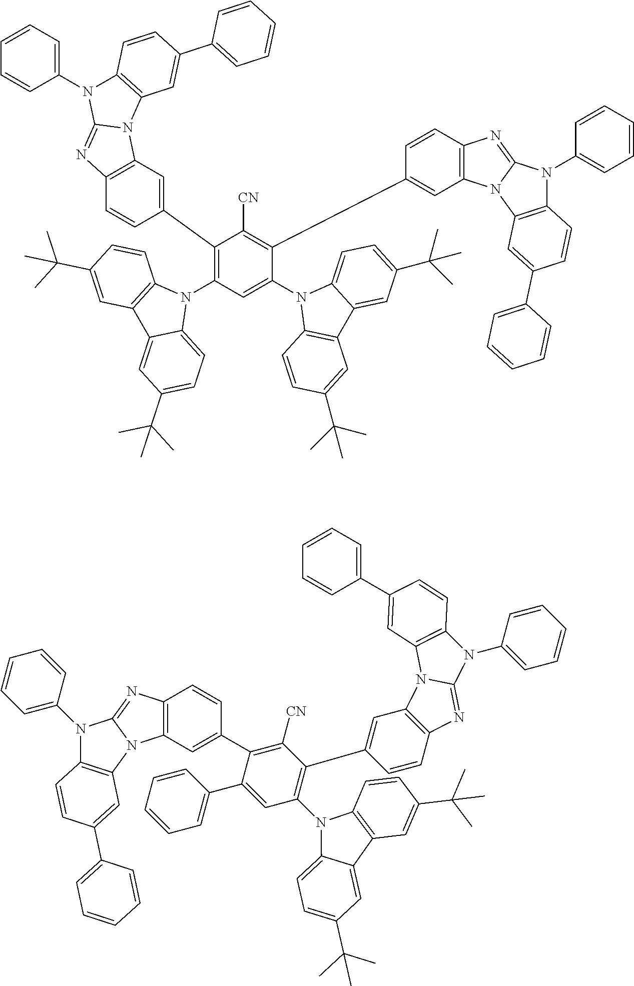 [SMILES: CC(C)(C)c1ccc2c(c1)c1cc(C(C)(C)C)ccc1n2-c1cc(-n2c3ccc(C(C)(C)C)cc3c3cc(C(C)(C)C)ccc32)c(-c2ccc3nc4n(-c5ccccc5)c5ccc(-c6ccccc6)cc5n4c3c2)c(C#N)c1-c1ccc2nc3n(-c4ccccc4)c4ccc(-c5ccccc5)cc4n3c2c1 ; CC(C)(C)c1ccc2c(c1)c1cc(C(C)(C)C)ccc1n2-c1cc(-c2ccccc2)c(-c2ccc3nc4n(-c5ccccc5)c5ccc(-c6ccccc6)cc5n4c3c2)c(C#N)c1-c1ccc2nc3n(-c4ccccc4)c4ccc(-c5ccccc5)cc4n3c2c1]

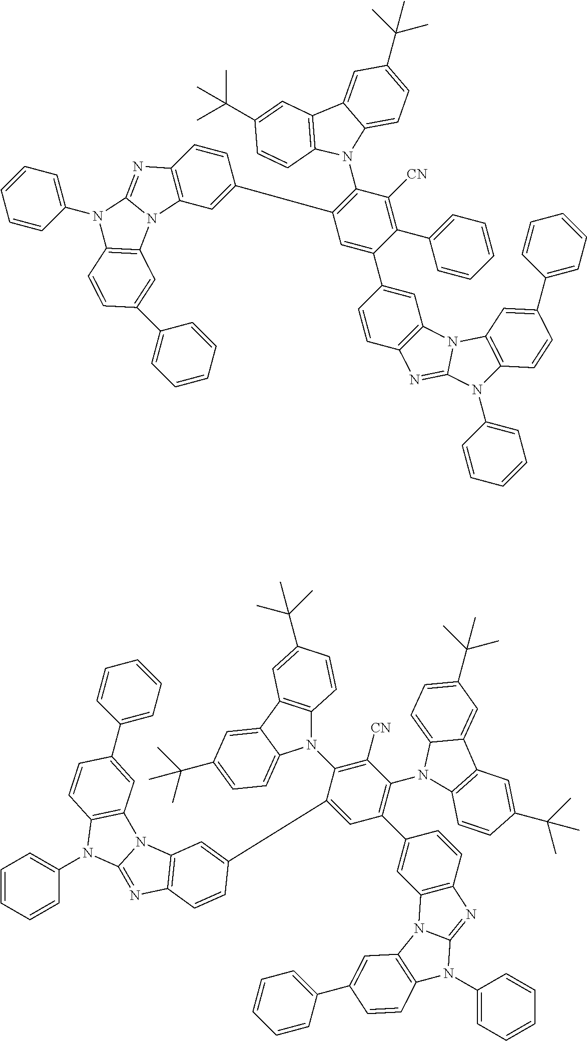 [SMILES: CC(C)(C)c1ccc2c(c1)c1cc(C(C)(C)C)ccc1n2-c1c(-c2ccc3nc4n(-c5ccccc5)c5ccc(-c6ccccc6)cc5n4c3c2)cc(-c2ccc3nc4n(-c5ccccc5)c5ccc(-c6ccccc6)cc5n4c3c2)c(-c2ccccc2)c1C#N ; CC(C)(C)c1ccc2c(c1)c1cc(C(C)(C)C)ccc1n2-c1c(-c2ccc3nc4n(-c5ccccc5)c5ccc(-c6ccccc6)cc5n4c3c2)cc(-c2ccc3nc4n(-c5ccccc5)c5ccc(-c6ccccc6)cc5n4c3c2)c(-n2c3ccc(C(C)(C)C)cc3c3cc(C(C)(C)C)ccc32)c1C#N]

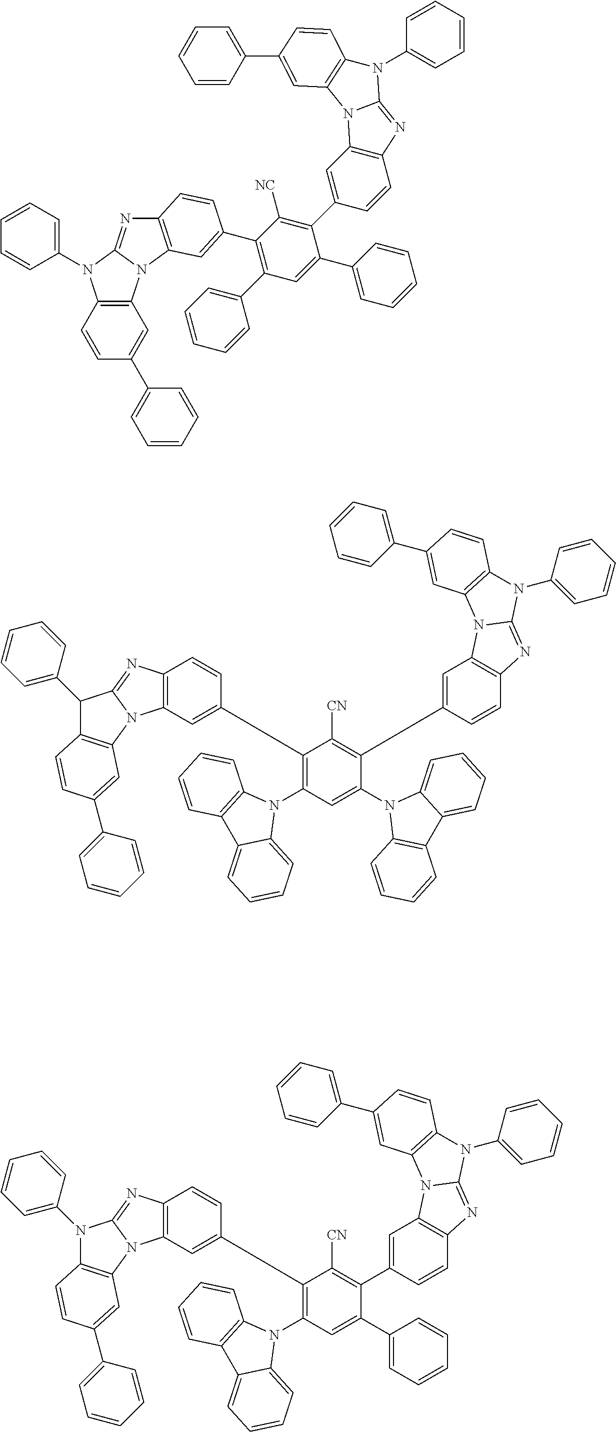 [SMILES: N#Cc1c(-c2ccc3nc4n(-c5ccccc5)c5ccc(-c6ccccc6)cc5n4c3c2)c(-c2ccccc2)cc(-c2ccccc2)c1-c1ccc2nc3n(-c4ccccc4)c4ccc(-c5ccccc5)cc4n3c2c1 ; N#Cc1c(-c2ccc3nc4n(c3c2)-c2cc(-c3ccccc3)ccc2C4c2ccccc2)c(-n2c3ccccc3c3ccccc32)cc(-n2c3ccccc3c3ccccc32)c1-c1ccc2nc3n(-c4ccccc4)c4ccc(-c5ccccc5)cc4n3c2c1 ; N#Cc1c(-c2ccc3nc4n(-c5ccccc5)c5ccc(-c6ccccc6)cc5n4c3c2)c(-c2ccccc2)cc(-n2c3ccccc3c3ccccc32)c1-c1ccc2nc3n(-c4ccccc4)c4ccc(-c5ccccc5)cc4n3c2c1]

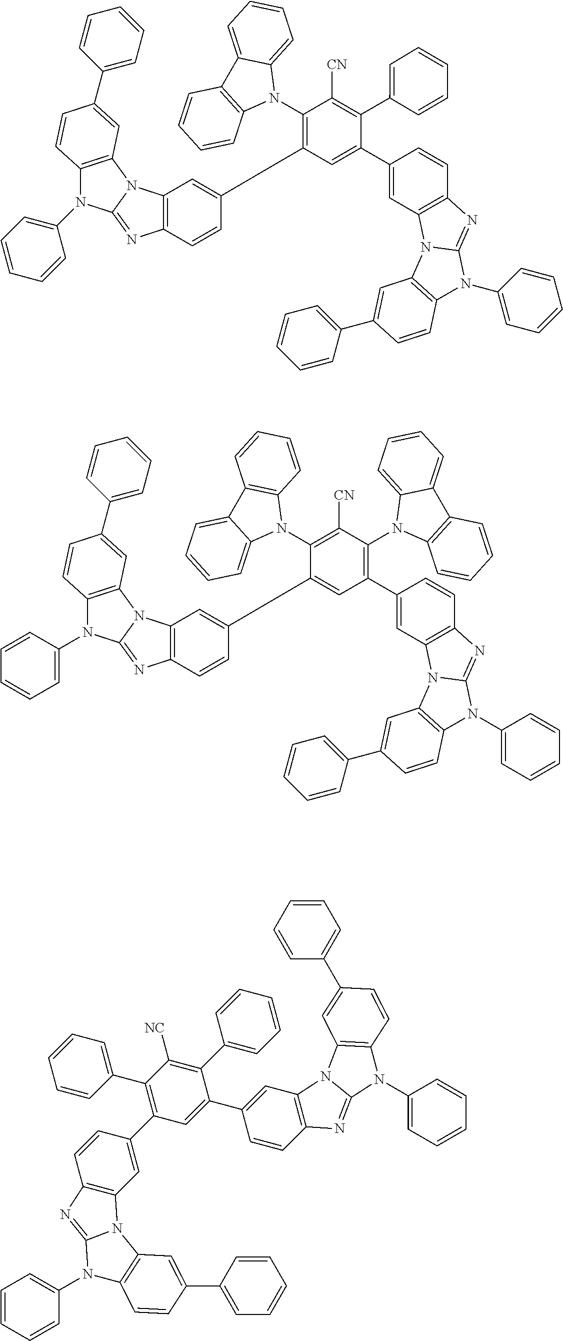 [SMILES: N#Cc1c(-c2ccccc2)c(-c2ccc3nc4n(-c5ccccc5)c5ccc(-c6ccccc6)cc5n4c3c2)cc(-c2ccc3nc4n(-c5ccccc5)c5ccc(-c6ccccc6)cc5n4c3c2)c1-n1c2ccccc2c2ccccc21 ; N#Cc1c(-n2c3ccccc3c3ccccc32)c(-c2ccc3nc4n(-c5ccccc5)c5ccc(-c6ccccc6)cc5n4c3c2)cc(-c2ccc3nc4n(-c5ccccc5)c5ccc(-c6ccccc6)cc5n4c3c2)c1-n1c2ccccc2c2ccccc21 ; N#Cc1c(-c2ccccc2)c(-c2ccc3nc4n(-c5ccccc5)c5ccc(-c6ccccc6)cc5n4c3c2)cc(-c2ccc3nc4n(-c5ccccc5)c5ccc(-c6ccccc6)cc5n4c3c2)c1-c1ccccc1]

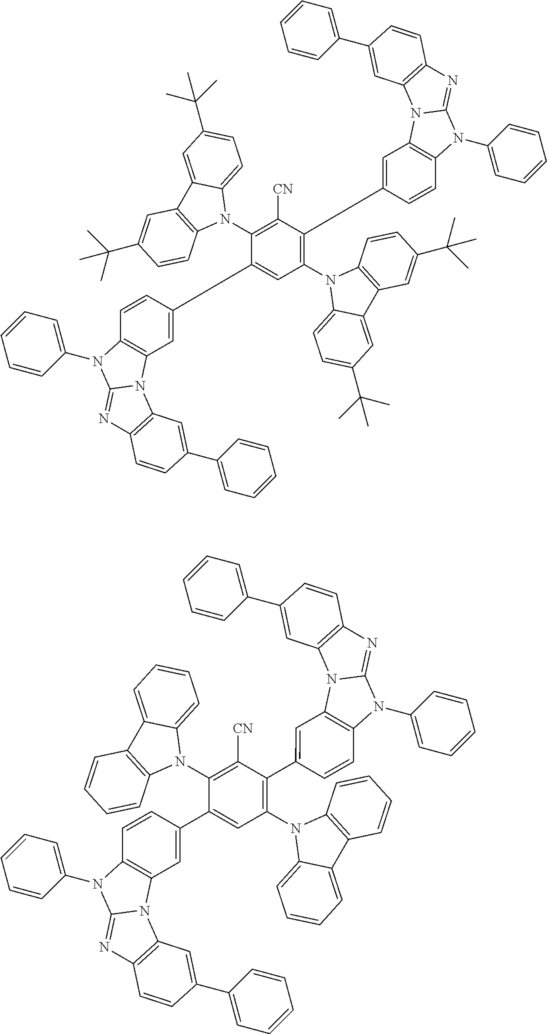 [SMILES: CC(C)(C)c1ccc2c(c1)c1cc(C(C)(C)C)ccc1n2-c1cc(-c2ccc3c(c2)n2c4cc(-c5ccccc5)ccc4nc2n3-c2ccccc2)c(-n2c3ccc(C(C)(C)C)cc3c3cc(C(C)(C)C)ccc32)c(C#N)c1-c1ccc2c(c1)n1c3cc(-c4ccccc4)ccc3nc1n2-c1ccccc1 ; N#Cc1c(-c2ccc3c(c2)n2c4cc(-c5ccccc5)ccc4nc2n3-c2ccccc2)c(-n2c3ccccc3c3ccccc32)cc(-c2ccc3c(c2)n2c4cc(-c5ccccc5)ccc4nc2n3-c2ccccc2)c1-n1c2ccccc2c2ccccc21]

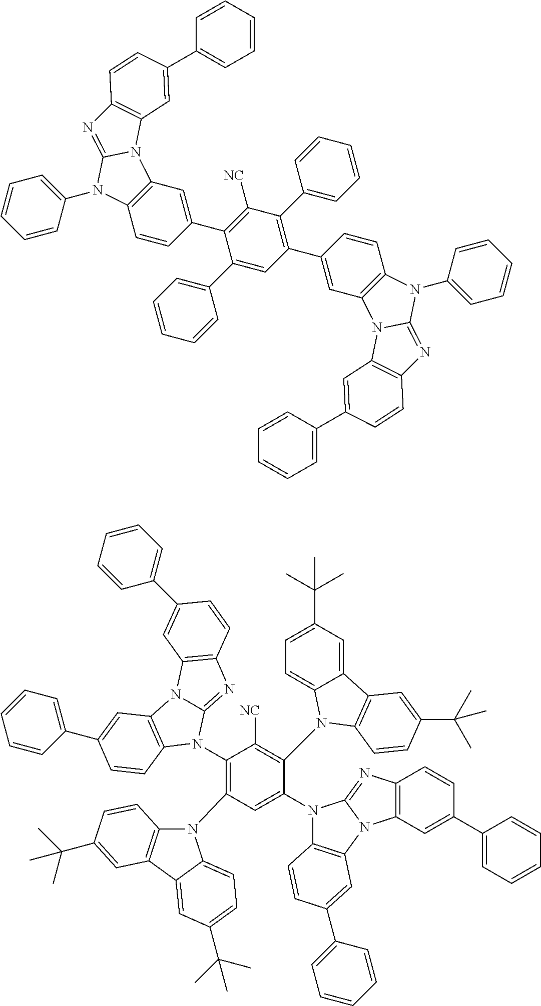 [SMILES: N#Cc1c(-c2ccccc2)c(-c2ccc3c(c2)n2c4cc(-c5ccccc5)ccc4nc2n3-c2ccccc2)cc(-c2ccccc2)c1-c1ccc2c(c1)n1c3cc(-c4ccccc4)ccc3nc1n2-c1ccccc1 ; CC(C)(C)c1ccc2c(c1)c1cc(C(C)(C)C)ccc1n2-c1cc(-n2c3ccc(-c4ccccc4)cc3n3c4cc(-c5ccccc5)ccc4nc23)c(-n2c3ccc(C(C)(C)C)cc3c3cc(C(C)(C)C)ccc32)c(C#N)c1-n1c2ccc(-c3ccccc3)cc2n2c3cc(-c4ccccc4)ccc3nc12]

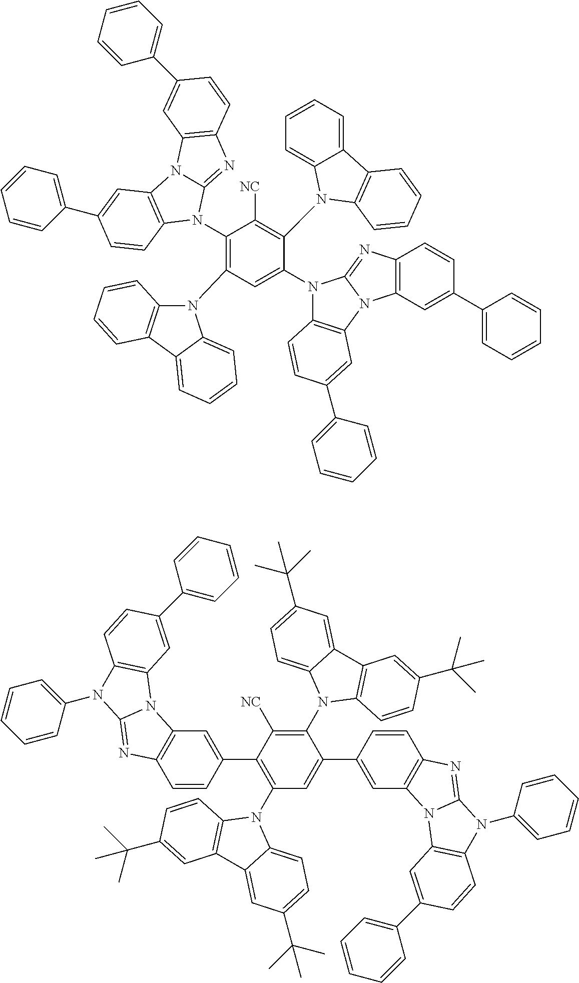 [SMILES: N#Cc1c(-n2c3ccccc3c3ccccc32)c(-n2c3ccc(-c4ccccc4)cc3n3c4cc(-c5ccccc5)ccc4nc23)cc(-n2c3ccccc3c3ccccc32)c1-n1c2ccc(-c3ccccc3)cc2n2c3cc(-c4ccccc4)ccc3nc12 ; CC(C)(C)c1ccc2c(c1)c1cc(C(C)(C)C)ccc1n2-c1cc(-c2ccc3nc4n(-c5ccccc5)c5ccc(-c6ccccc6)cc5n4c3c2)c(-n2c3ccc(C(C)(C)C)cc3c3cc(C(C)(C)C)ccc32)c(C#N)c1-c1ccc2nc3n(-c4ccccc4)c4ccc(-c5ccccc5)cc4n3c2c1]

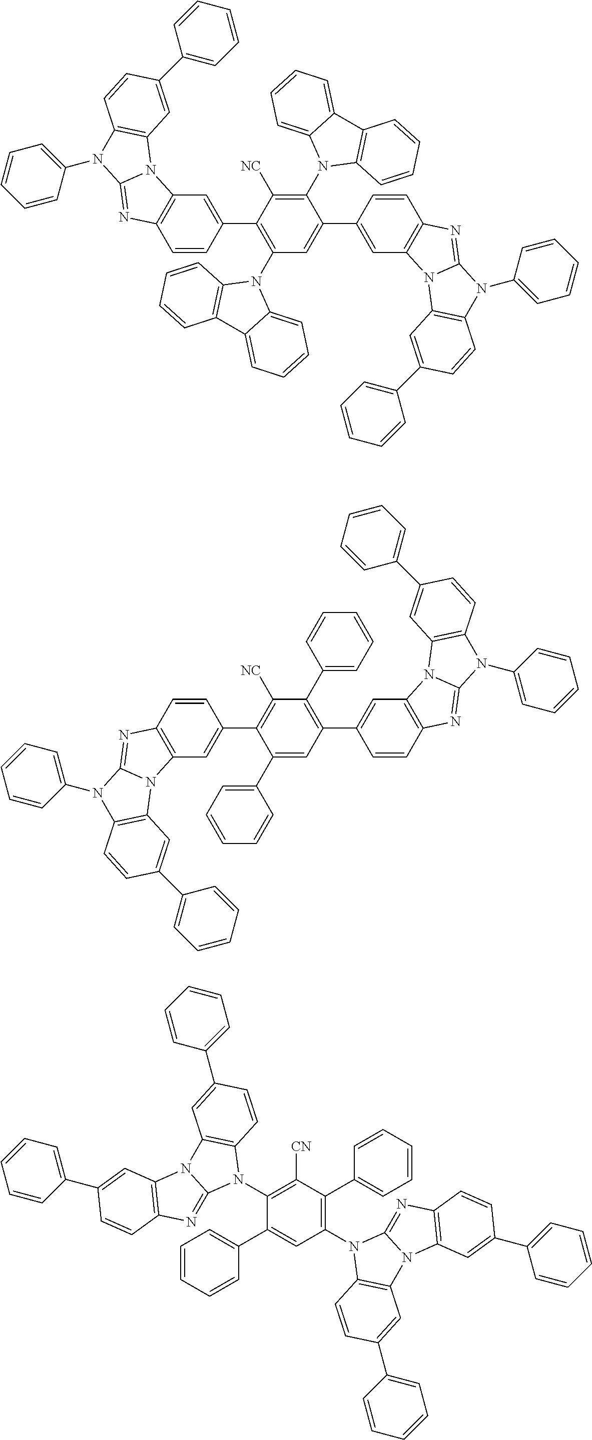

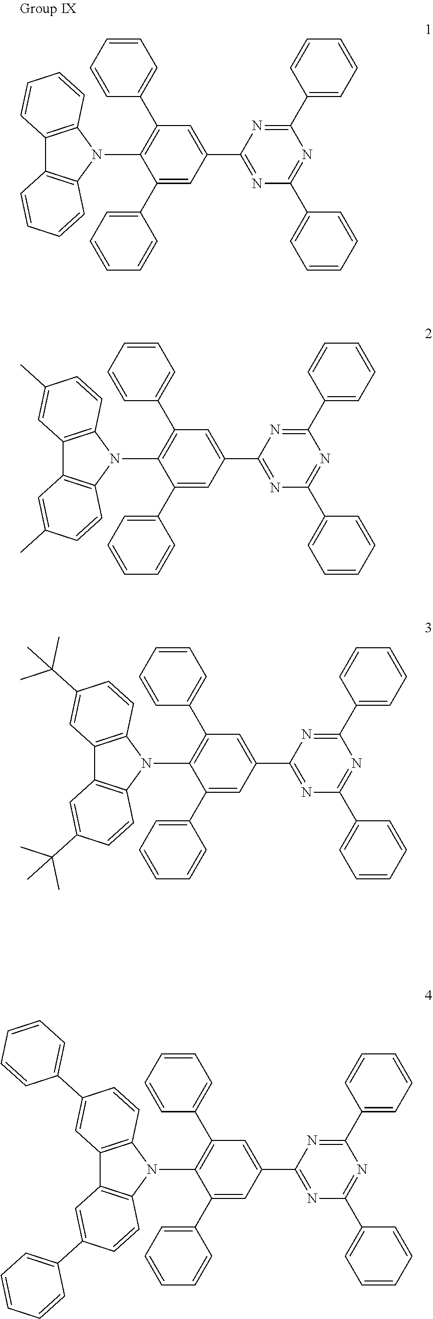 [SMILES: c1ccc(-c2nc(-c3ccccc3)nc(-c3cc(-c4ccccc4)c(-n4c5ccccc5c5ccccc54)c(-c4ccccc4)c3)n2)cc1 ; Cc1ccc2c(c1)c1cc(C)ccc1n2-c1c(-c2ccccc2)cc(-c2nc(-c3ccccc3)nc(-c3ccccc3)n2)cc1-c1ccccc1 ; CC(C)(C)c1ccc2c(c1)c1cc(C(C)(C)C)ccc1n2-c1c(-c2ccccc2)cc(-c2nc(-c3ccccc3)nc(-c3ccccc3)n2)cc1-c1ccccc1 ; c1ccc(-c2ccc3c(c2)c2cc(-c4ccccc4)ccc2n3-c2c(-c3ccccc3)cc(-c3nc(-c4ccccc4)nc(-c4ccccc4)n3)cc2-c2ccccc2)cc1]

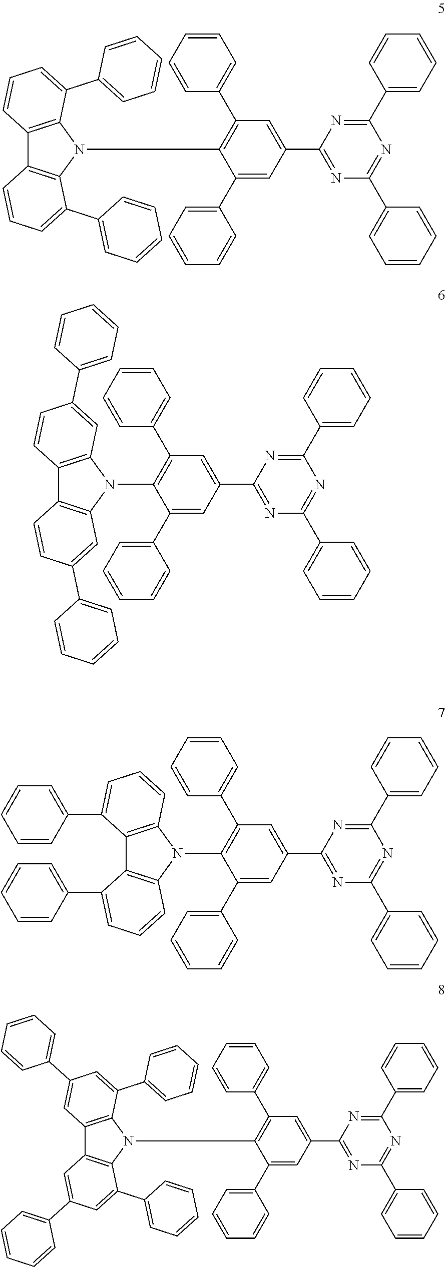 [SMILES: c1ccc(-c2nc(-c3ccccc3)nc(-c3cc(-c4ccccc4)c(-n4c5c(-c6ccccc6)cccc5c5cccc(-c6ccccc6)c54)c(-c4ccccc4)c3)n2)cc1 ; c1ccc(-c2ccc3c4ccc(-c5ccccc5)cc4n(-c4c(-c5ccccc5)cc(-c5nc(-c6ccccc6)nc(-c6ccccc6)n5)cc4-c4ccccc4)c3c2)cc1 ; c1ccc(-c2nc(-c3ccccc3)nc(-c3cc(-c4ccccc4)c(-n4c5cccc(-c6ccccc6)c5c5c(-c6ccccc6)cccc54)c(-c4ccccc4)c3)n2)cc1 ; c1ccc(-c2cc(-c3ccccc3)c3c(c2)c2cc(-c4ccccc4)cc(-c4ccccc4)c2n3-c2c(-c3ccccc3)cc(-c3nc(-c4ccccc4)nc(-c4ccccc4)n3)cc2-c2ccccc2)cc1]

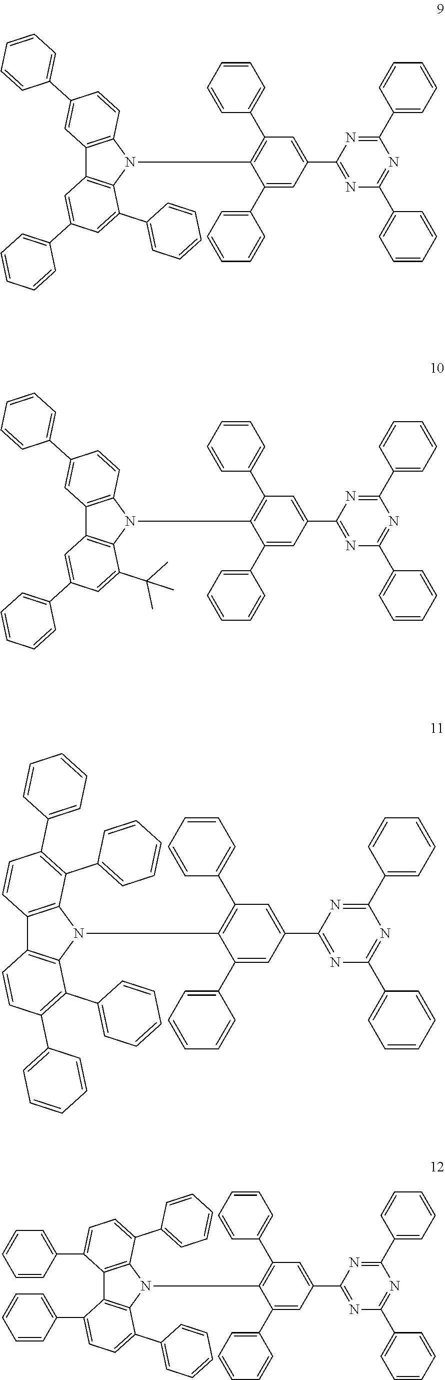 [SMILES: c1ccc(-c2ccc3c(c2)c2cc(-c4ccccc4)cc(-c4ccccc4)c2n3-c2c(-c3ccccc3)cc(-c3nc(-c4ccccc4)nc(-c4ccccc4)n3)cc2-c2ccccc2)cc1 ; CC(C)(C)c1cc(-c2ccccc2)cc2c3cc(-c4ccccc4)ccc3n(-c3c(-c4ccccc4)cc(-c4nc(-c5ccccc5)nc(-c5ccccc5)n4)cc3-c3ccccc3)c12 ; c1ccc(-c2nc(-c3ccccc3)nc(-c3cc(-c4ccccc4)c(-n4c5c(-c6ccccc6)c(-c6ccccc6)ccc5c5ccc(-c6ccccc6)c(-c6ccccc6)c54)c(-c4ccccc4)c3)n2)cc1 ; c1ccc(-c2nc(-c3ccccc3)nc(-c3cc(-c4ccccc4)c(-n4c5c(-c6ccccc6)ccc(-c6ccccc6)c5c5c(-c6ccccc6)ccc(-c6ccccc6)c54)c(-c4ccccc4)c3)n2)cc1]

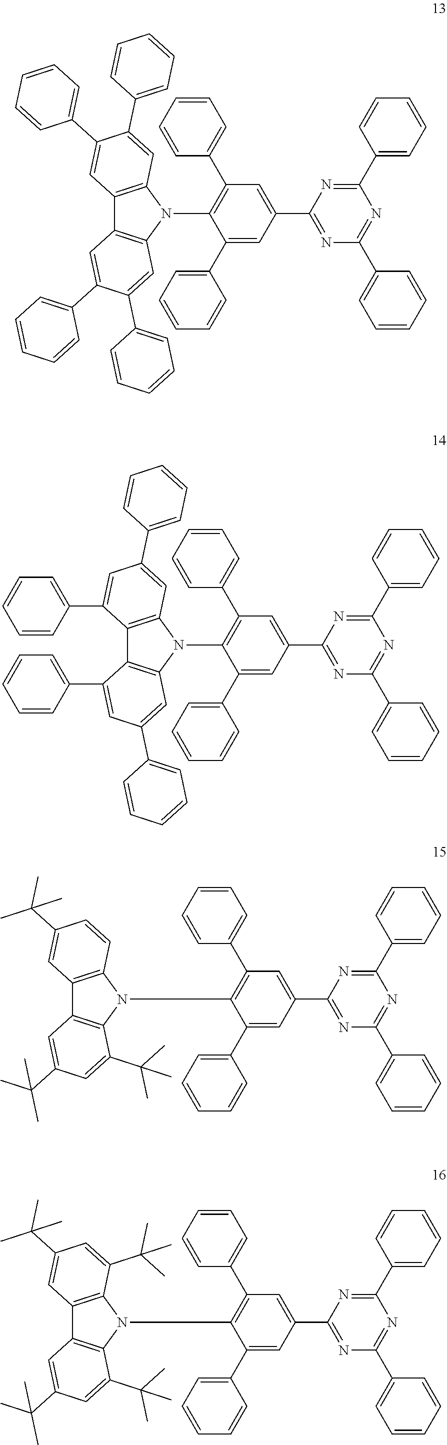 [SMILES: c1ccc(-c2nc(-c3ccccc3)nc(-c3cc(-c4ccccc4)c(-n4c5cc(-c6ccccc6)c(-c6ccccc6)cc5c5cc(-c6ccccc6)c(-c6ccccc6)cc54)c(-c4ccccc4)c3)n2)cc1 ; c1ccc(-c2cc(-c3ccccc3)c3c4c(-c5ccccc5)cc(-c5ccccc5)cc4n(-c4c(-c5ccccc5)cc(-c5nc(-c6ccccc6)nc(-c6ccccc6)n5)cc4-c4ccccc4)c3c2)cc1 ; CC(C)(C)c1ccc2c(c1)c1cc(C(C)(C)C)cc(C(C)(C)C)c1n2-c1c(-c2ccccc2)cc(-c2nc(-c3ccccc3)nc(-c3ccccc3)n2)cc1-c1ccccc1 ; CC(C)(C)c1cc(C(C)(C)C)c2c(c1)c1cc(C(C)(C)C)cc(C(C)(C)C)c1n2-c1c(-c2ccccc2)cc(-c2nc(-c3ccccc3)nc(-c3ccccc3)n2)cc1-c1ccccc1]

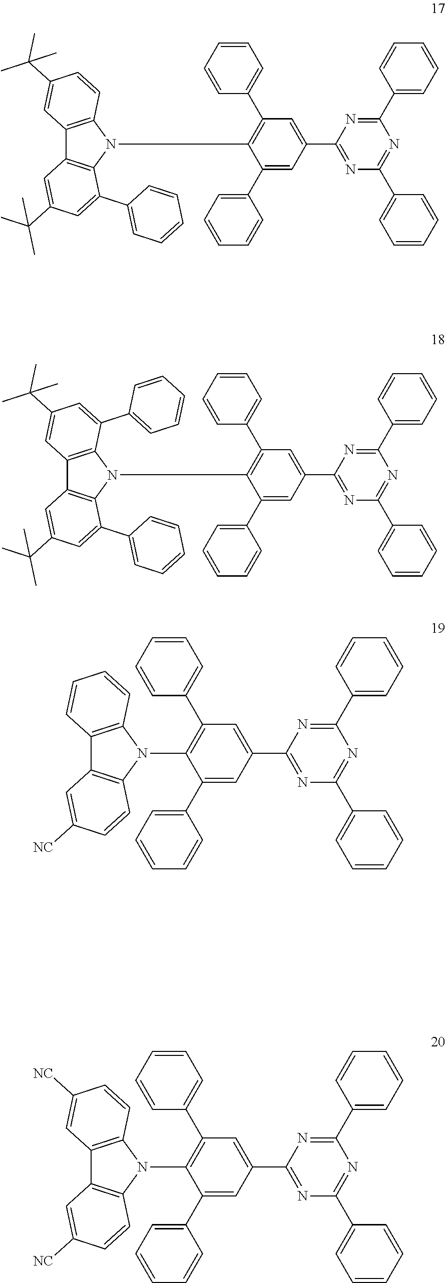 [SMILES: CC(C)(C)c1ccc2c(c1)c1cc(C(C)(C)C)cc(-c3ccccc3)c1n2-c1c(-c2ccccc2)cc(-c2nc(-c3ccccc3)nc(-c3ccccc3)n2)cc1-c1ccccc1 ; CC(C)(C)c1cc(-c2ccccc2)c2c(c1)c1cc(C(C)(C)C)cc(-c3ccccc3)c1n2-c1c(-c2ccccc2)cc(-c2nc(-c3ccccc3)nc(-c3ccccc3)n2)cc1-c1ccccc1 ; N#Cc1ccc2c(c1)c1ccccc1n2-c1c(-c2ccccc2)cc(-c2nc(-c3ccccc3)nc(-c3ccccc3)n2)cc1-c1ccccc1 ; N#Cc1ccc2c(c1)c1cc(C#N)ccc1n2-c1c(-c2ccccc2)cc(-c2nc(-c3ccccc3)nc(-c3ccccc3)n2)cc1-c1ccccc1]

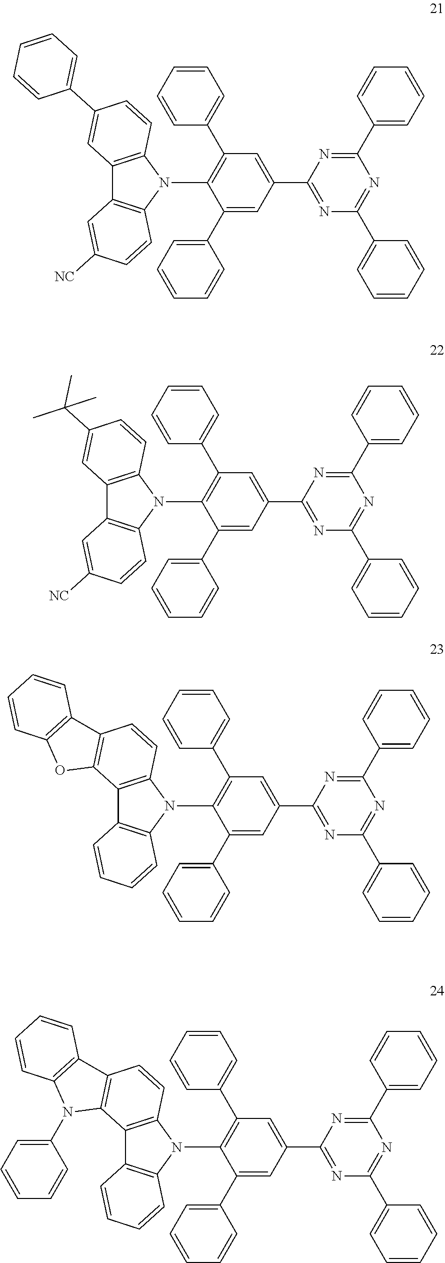 [SMILES: N#Cc1ccc2c(c1)c1cc(-c3ccccc3)ccc1n2-c1c(-c2ccccc2)cc(-c2nc(-c3ccccc3)nc(-c3ccccc3)n2)cc1-c1ccccc1 ; CC(C)(C)c1ccc2c(c1)c1cc(C#N)ccc1n2-c1c(-c2ccccc2)cc(-c2nc(-c3ccccc3)nc(-c3ccccc3)n2)cc1-c1ccccc1 ; c1ccc(-c2nc(-c3ccccc3)nc(-c3cc(-c4ccccc4)c(-n4c5ccccc5c5c6oc7ccccc7c6ccc54)c(-c4ccccc4)c3)n2)cc1 ; c1ccc(-c2nc(-c3ccccc3)nc(-c3cc(-c4ccccc4)c(-n4c5ccccc5c5c4ccc4c6ccccc6n(-c6ccccc6)c45)c(-c4ccccc4)c3)n2)cc1]

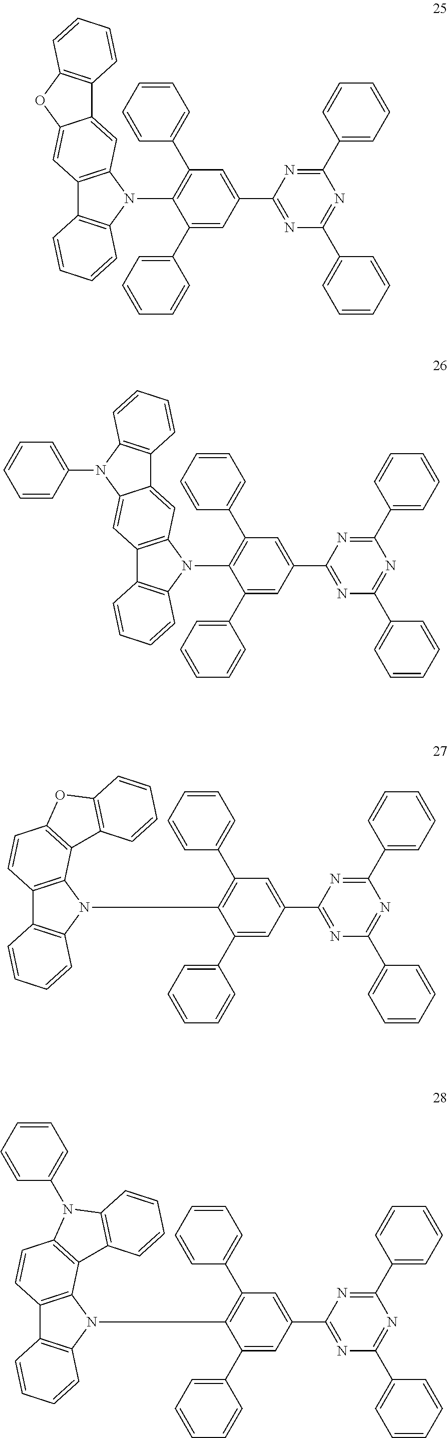 [SMILES: c1ccc(-c2nc(-c3ccccc3)nc(-c3cc(-c4ccccc4)c(-n4c5ccccc5c5cc6oc7ccccc7c6cc54)c(-c4ccccc4)c3)n2)cc1 ; c1ccc(-c2nc(-c3ccccc3)nc(-c3cc(-c4ccccc4)c(-n4c5ccccc5c5cc6c(cc54)c4ccccc4n6-c4ccccc4)c(-c4ccccc4)c3)n2)cc1 ; c1ccc(-c2nc(-c3ccccc3)nc(-c3cc(-c4ccccc4)c(-n4c5ccccc5c5ccc6oc7ccccc7c6c54)c(-c4ccccc4)c3)n2)cc1 ; c1ccc(-c2nc(-c3ccccc3)nc(-c3cc(-c4ccccc4)c(-n4c5ccccc5c5ccc6c(c7ccccc7n6-c6ccccc6)c54)c(-c4ccccc4)c3)n2)cc1]

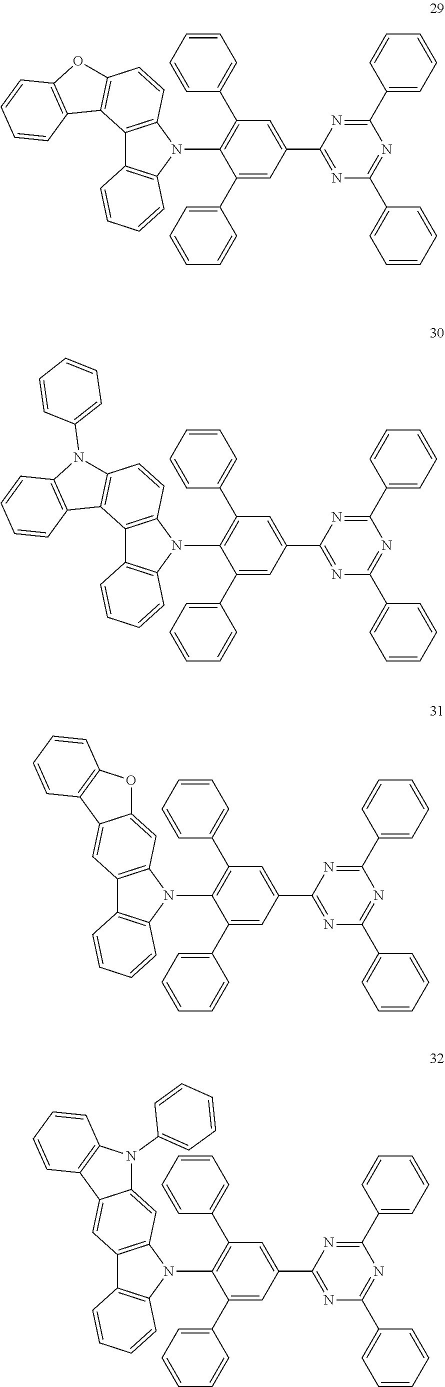 [SMILES: c1ccc(-c2nc(-c3ccccc3)nc(-c3cc(-c4ccccc4)c(-n4c5ccccc5c5c6c(ccc54)oc4ccccc46)c(-c4ccccc4)c3)n2)cc1 ; c1ccc(-c2nc(-c3ccccc3)nc(-c3cc(-c4ccccc4)c(-n4c5ccccc5c5c6c7ccccc7n(-c7ccccc7)c6ccc54)c(-c4ccccc4)c3)n2)cc1 ; c1ccc(-c2nc(-c3ccccc3)nc(-c3cc(-c4ccccc4)c(-n4c5ccccc5c5cc6c(cc54)oc4ccccc46)c(-c4ccccc4)c3)n2)cc1 ; c1ccc(-c2nc(-c3ccccc3)nc(-c3cc(-c4ccccc4)c(-n4c5ccccc5c5cc6c7ccccc7n(-c7ccccc7)c6cc54)c(-c4ccccc4)c3)n2)cc1]

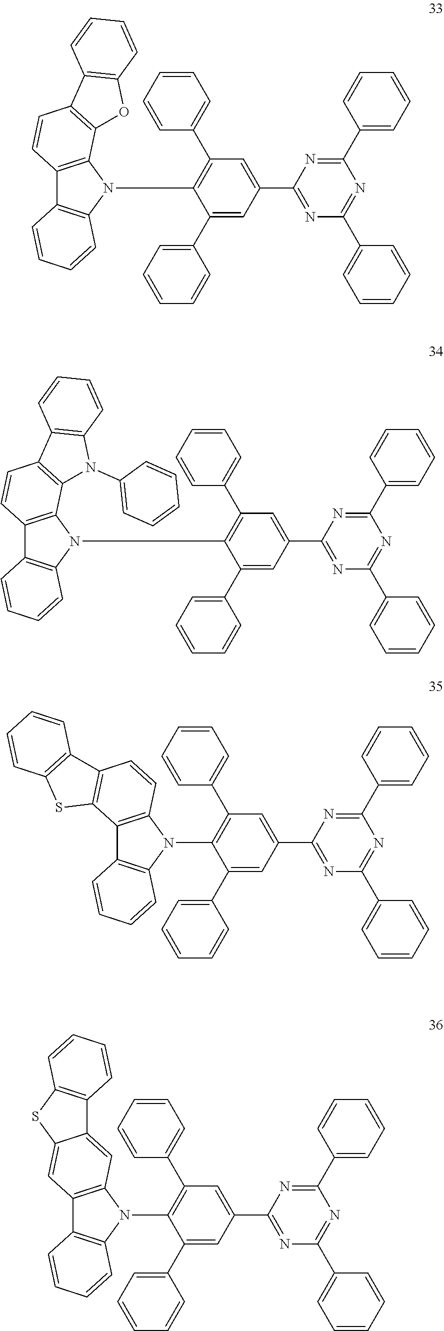 [SMILES: c1ccc(-c2nc(-c3ccccc3)nc(-c3cc(-c4ccccc4)c(-n4c5ccccc5c5ccc6c7ccccc7oc6c54)c(-c4ccccc4)c3)n2)cc1 ; c1ccc(-c2nc(-c3ccccc3)nc(-c3cc(-c4ccccc4)c(-n4c5ccccc5c5ccc6c7ccccc7n(-c7ccccc7)c6c54)c(-c4ccccc4)c3)n2)cc1 ; c1ccc(-c2nc(-c3ccccc3)nc(-c3cc(-c4ccccc4)c(-n4c5ccccc5c5c6sc7ccccc7c6ccc54)c(-c4ccccc4)c3)n2)cc1 ; c1ccc(-c2nc(-c3ccccc3)nc(-c3cc(-c4ccccc4)c(-n4c5ccccc5c5cc6sc7ccccc7c6cc54)c(-c4ccccc4)c3)n2)cc1]

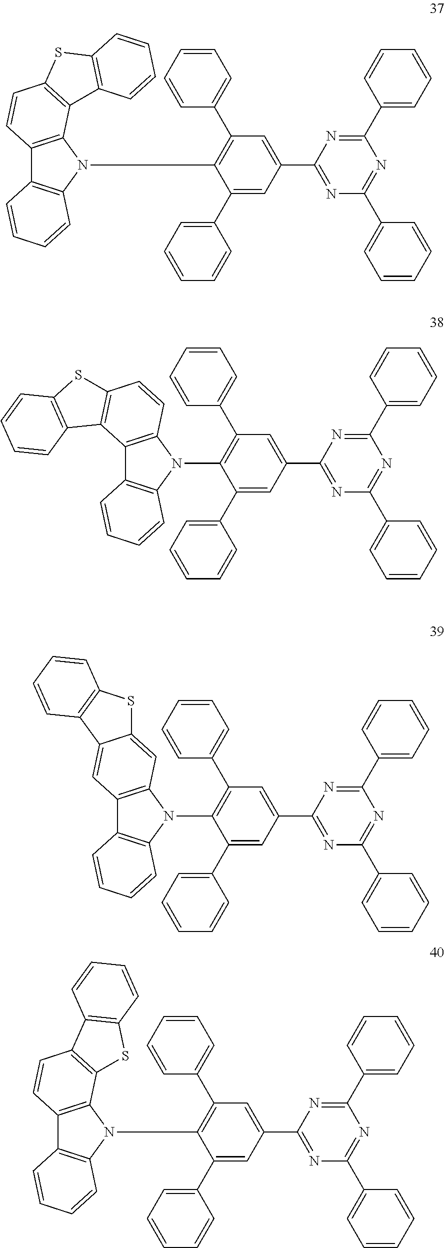 [SMILES: c1ccc(-c2nc(-c3ccccc3)nc(-c3cc(-c4ccccc4)c(-n4c5ccccc5c5ccc6sc7ccccc7c6c54)c(-c4ccccc4)c3)n2)cc1 ; c1ccc(-c2nc(-c3ccccc3)nc(-c3cc(-c4ccccc4)c(-n4c5ccccc5c5c6c(ccc54)sc4ccccc46)c(-c4ccccc4)c3)n2)cc1 ; c1ccc(-c2nc(-c3ccccc3)nc(-c3cc(-c4ccccc4)c(-n4c5ccccc5c5cc6c(cc54)sc4ccccc46)c(-c4ccccc4)c3)n2)cc1 ; c1ccc(-c2nc(-c3ccccc3)nc(-c3cc(-c4ccccc4)c(-n4c5ccccc5c5ccc6c7ccccc7sc6c54)c(-c4ccccc4)c3)n2)cc1]

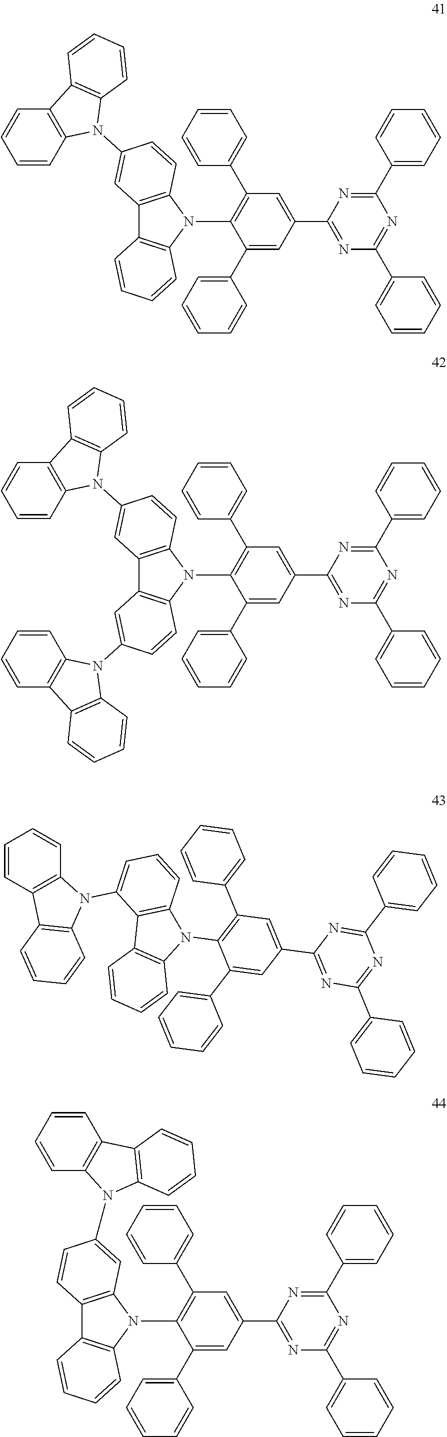 [SMILES: c1ccc(-c2nc(-c3ccccc3)nc(-c3cc(-c4ccccc4)c(-n4c5ccccc5c5cc(-n6c7ccccc7c7ccccc76)ccc54)c(-c4ccccc4)c3)n2)cc1 ; c1ccc(-c2nc(-c3ccccc3)nc(-c3cc(-c4ccccc4)c(-n4c5ccc(-n6c7ccccc7c7ccccc76)cc5c5cc(-n6c7ccccc7c7ccccc76)ccc54)c(-c4ccccc4)c3)n2)cc1 ; c1ccc(-c2nc(-c3ccccc3)nc(-c3cc(-c4ccccc4)c(-n4c5ccccc5c5c(-n6c7ccccc7c7ccccc76)cccc54)c(-c4ccccc4)c3)n2)cc1 ; c1ccc(-c2nc(-c3ccccc3)nc(-c3cc(-c4ccccc4)c(-n4c5ccccc5c5ccc(-n6c7ccccc7c7ccccc76)cc54)c(-c4ccccc4)c3)n2)cc1]

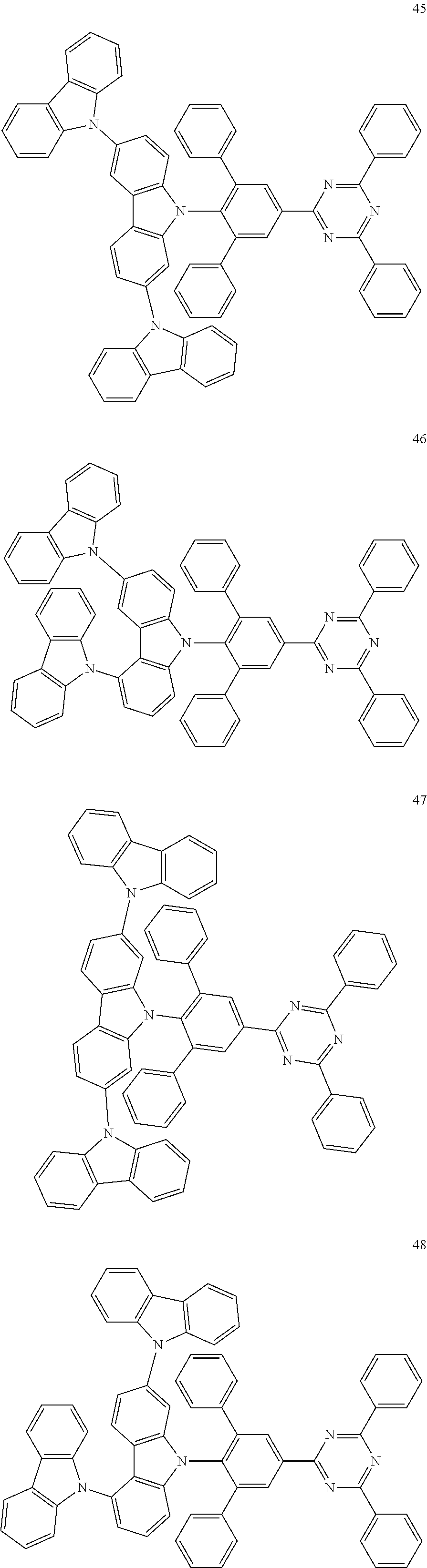

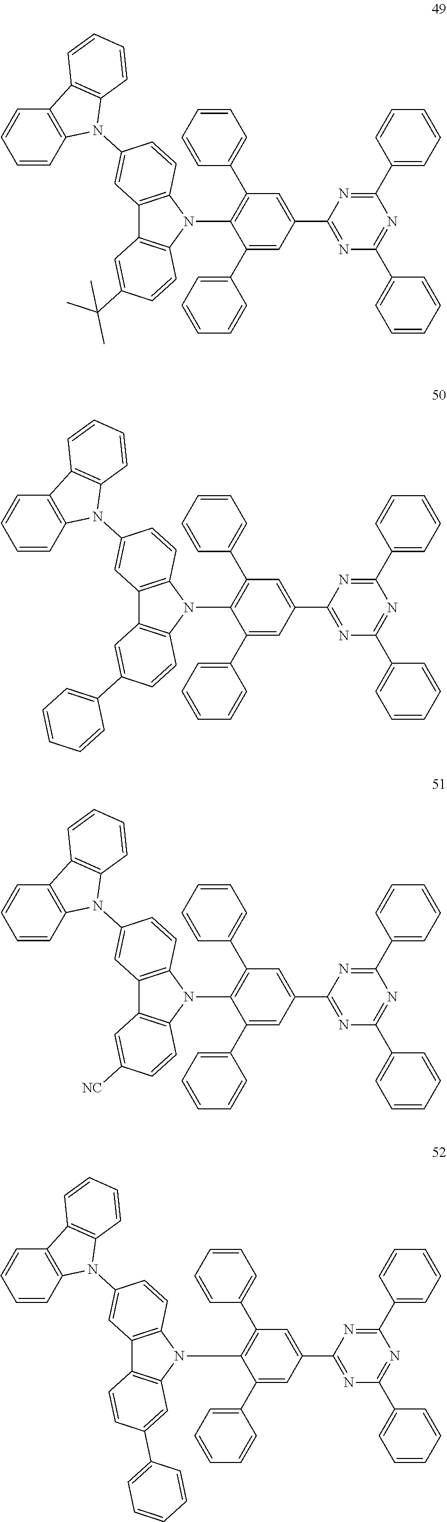 [SMILES: CC(C)(C)c1ccc2c(c1)c1cc(-n3c4ccccc4c4ccccc43)ccc1n2-c1c(-c2ccccc2)cc(-c2nc(-c3ccccc3)nc(-c3ccccc3)n2)cc1-c1ccccc1 ; c1ccc(-c2ccc3c(c2)c2cc(-n4c5ccccc5c5ccccc54)ccc2n3-c2c(-c3ccccc3)cc(-c3nc(-c4ccccc4)nc(-c4ccccc4)n3)cc2-c2ccccc2)cc1 ; N#Cc1ccc2c(c1)c1cc(-n3c4ccccc4c4ccccc43)ccc1n2-c1c(-c2ccccc2)cc(-c2nc(-c3ccccc3)nc(-c3ccccc3)n2)cc1-c1ccccc1 ; c1ccc(-c2ccc3c4cc(-n5c6ccccc6c6ccccc65)ccc4n(-c4c(-c5ccccc5)cc(-c5nc(-c6ccccc6)nc(-c6ccccc6)n5)cc4-c4ccccc4)c3c2)cc1]

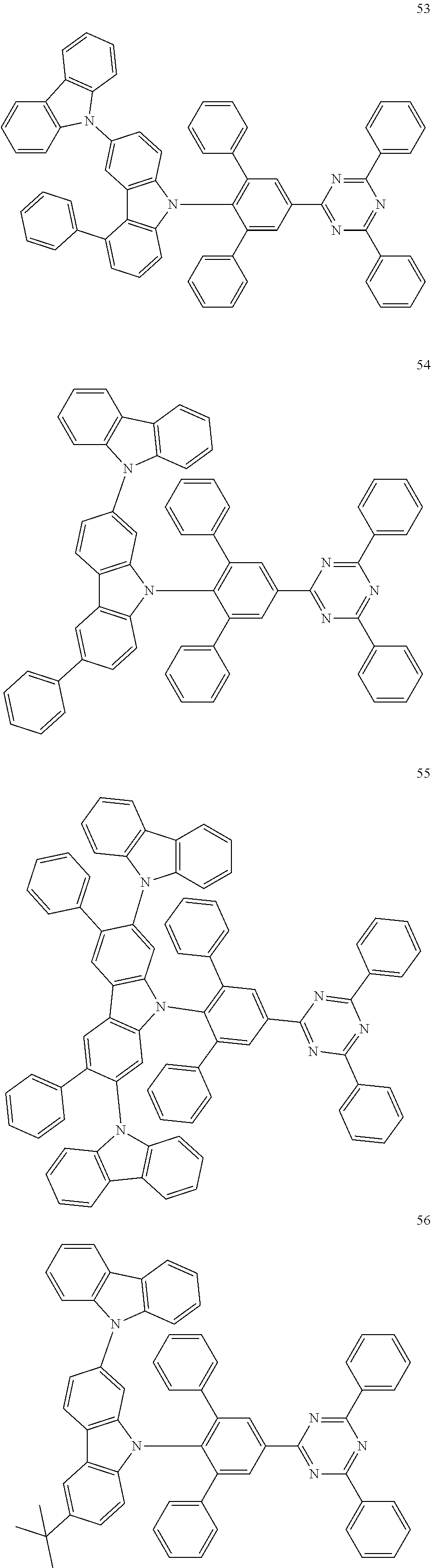

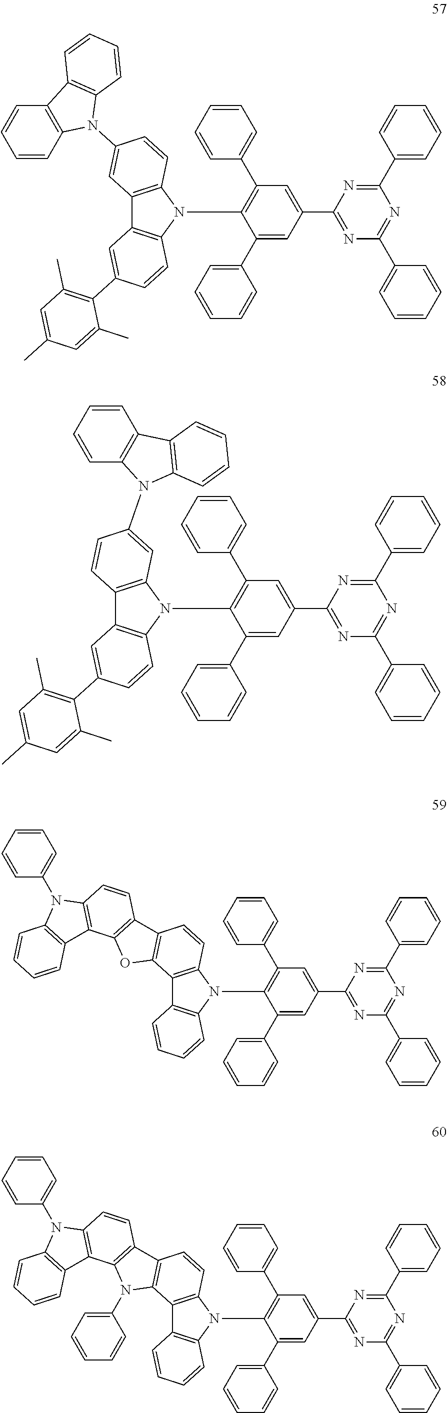

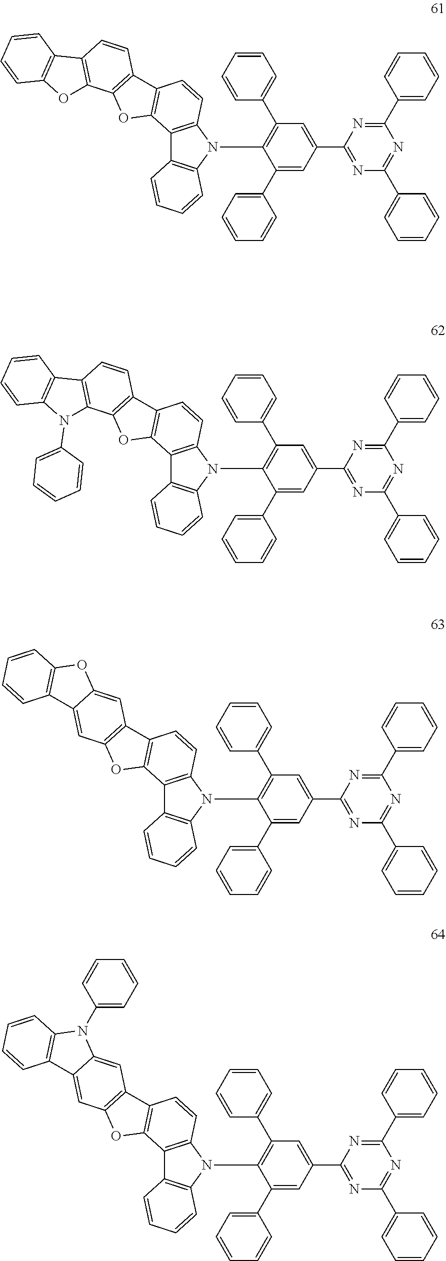 [SMILES: c1ccc(-c2nc(-c3ccccc3)nc(-c3cc(-c4ccccc4)c(-n4c5ccccc5c5c6oc7c(ccc8c9ccccc9oc87)c6ccc54)c(-c4ccccc4)c3)n2)cc1 ; c1ccc(-c2nc(-c3ccccc3)nc(-c3cc(-c4ccccc4)c(-n4c5ccccc5c5c6oc7c(ccc8c9ccccc9n(-c9ccccc9)c87)c6ccc54)c(-c4ccccc4)c3)n2)cc1 ; c1ccc(-c2nc(-c3ccccc3)nc(-c3cc(-c4ccccc4)c(-n4c5ccccc5c5c6oc7cc8c(cc7c6ccc54)oc4ccccc48)c(-c4ccccc4)c3)n2)cc1 ; c1ccc(-c2nc(-c3ccccc3)nc(-c3cc(-c4ccccc4)c(-n4c5ccccc5c5c6oc7cc8c9ccccc9n(-c9ccccc9)c8cc7c6ccc54)c(-c4ccccc4)c3)n2)cc1]

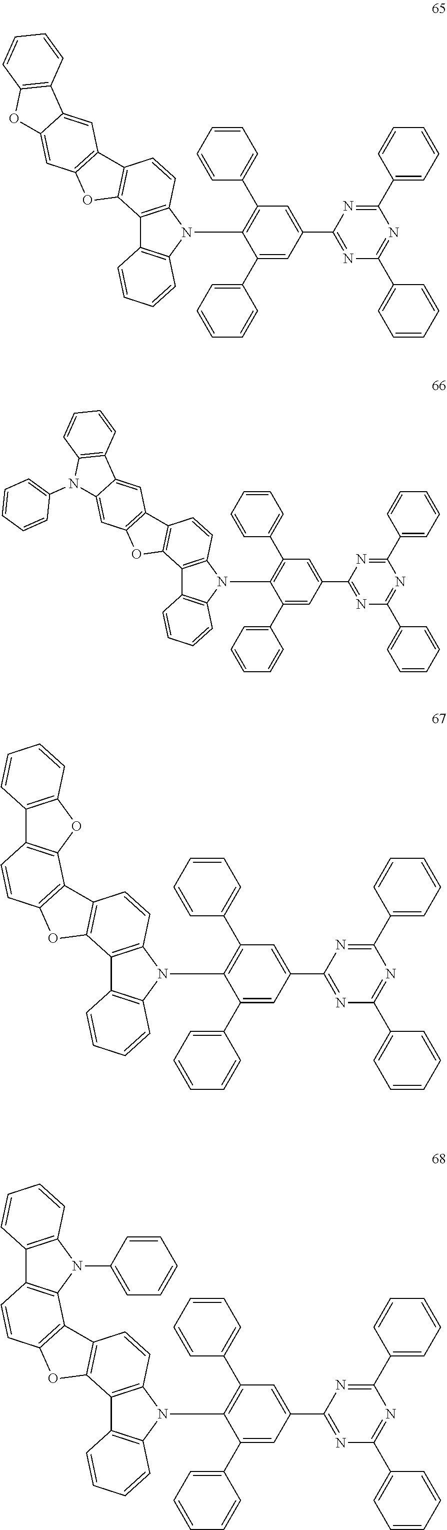 [SMILES: c1ccc(-c2nc(-c3ccccc3)nc(-c3cc(-c4ccccc4)c(-n4c5ccccc5c5c6oc7cc8oc9ccccc9c8cc7c6ccc54)c(-c4ccccc4)c3)n2)cc1 ; c1ccc(-c2nc(-c3ccccc3)nc(-c3cc(-c4ccccc4)c(-n4c5ccccc5c5c6oc7cc8c(cc7c6ccc54)c4ccccc4n8-c4ccccc4)c(-c4ccccc4)c3)n2)cc1 ; c1ccc(-c2nc(-c3ccccc3)nc(-c3cc(-c4ccccc4)c(-n4c5ccccc5c5c6oc7ccc8c9ccccc9oc8c7c6ccc54)c(-c4ccccc4)c3)n2)cc1 ; c1ccc(-c2nc(-c3ccccc3)nc(-c3cc(-c4ccccc4)c(-n4c5ccccc5c5c6oc7ccc8c9ccccc9n(-c9ccccc9)c8c7c6ccc54)c(-c4ccccc4)c3)n2)cc1]

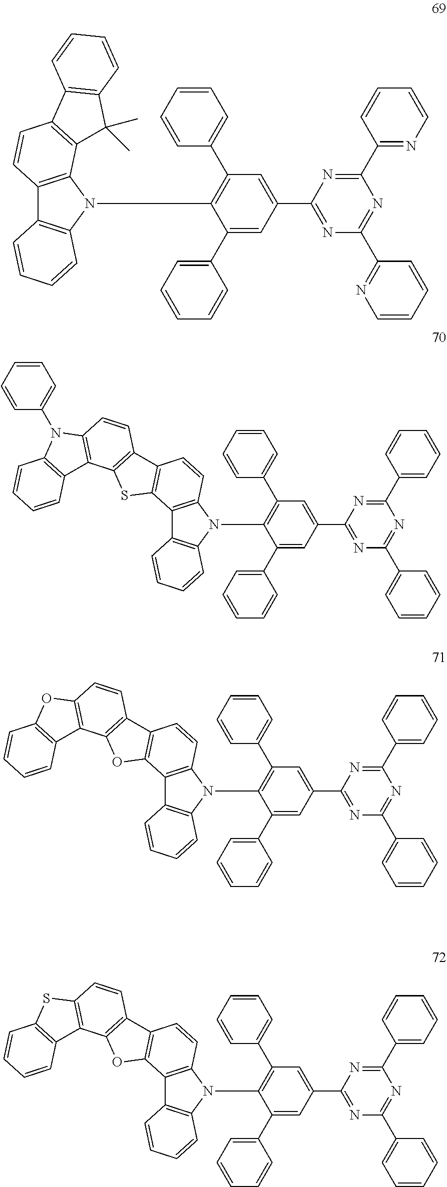 [SMILES: CC1(C)c2ccccc2-c2ccc3c4ccccc4n(-c4c(-c5ccccc5)cc(-c5nc(-c6ccccn6)nc(-c6ccccn6)n5)cc4-c4ccccc4)c3c21 ; c1ccc(-c2nc(-c3ccccc3)nc(-c3cc(-c4ccccc4)c(-n4c5ccccc5c5c6sc7c(ccc8c7c7ccccc7n8-c7ccccc7)c6ccc54)c(-c4ccccc4)c3)n2)cc1 ; c1ccc(-c2nc(-c3ccccc3)nc(-c3cc(-c4ccccc4)c(-n4c5ccccc5c5c6oc7c(ccc8oc9ccccc9c87)c6ccc54)c(-c4ccccc4)c3)n2)cc1 ; c1ccc(-c2nc(-c3ccccc3)nc(-c3cc(-c4ccccc4)c(-n4c5ccccc5c5c6oc7c(ccc8sc9ccccc9c87)c6ccc54)c(-c4ccccc4)c3)n2)cc1]

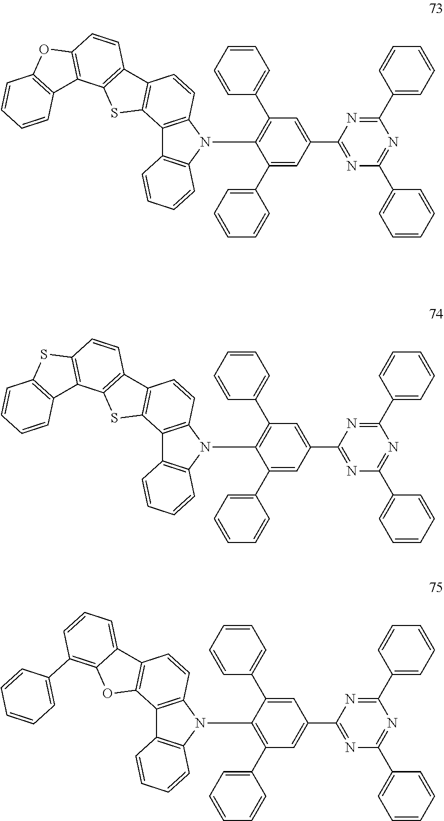 [SMILES: c1ccc(-c2nc(-c3ccccc3)nc(-c3cc(-c4ccccc4)c(-n4c5ccccc5c5c6sc7c(ccc8oc9ccccc9c87)c6ccc54)c(-c4ccccc4)c3)n2)cc1 ; c1ccc(-c2nc(-c3ccccc3)nc(-c3cc(-c4ccccc4)c(-n4c5ccccc5c5c6sc7c(ccc8sc9ccccc9c87)c6ccc54)c(-c4ccccc4)c3)n2)cc1 ; c1ccc(-c2nc(-c3ccccc3)nc(-c3cc(-c4ccccc4)c(-n4c5ccccc5c5c6oc7c(-c8ccccc8)cccc7c6ccc54)c(-c4ccccc4)c3)n2)cc1]

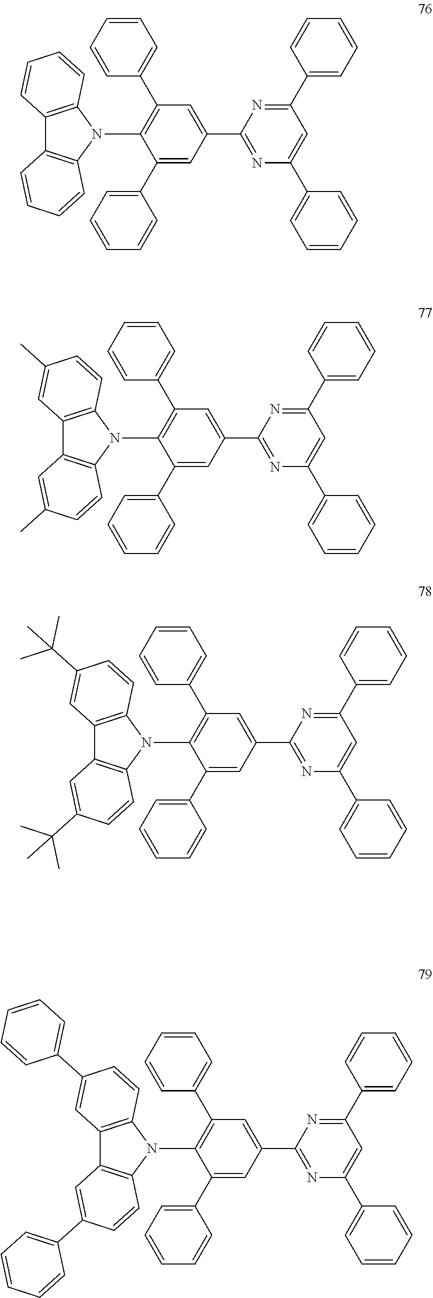 [SMILES: c1ccc(-c2cc(-c3ccccc3)nc(-c3cc(-c4ccccc4)c(-n4c5ccccc5c5ccccc54)c(-c4ccccc4)c3)n2)cc1 ; Cc1ccc2c(c1)c1cc(C)ccc1n2-c1c(-c2ccccc2)cc(-c2nc(-c3ccccc3)cc(-c3ccccc3)n2)cc1-c1ccccc1 ; CC(C)(C)c1ccc2c(c1)c1cc(C(C)(C)C)ccc1n2-c1c(-c2ccccc2)cc(-c2nc(-c3ccccc3)cc(-c3ccccc3)n2)cc1-c1ccccc1 ; c1ccc(-c2ccc3c(c2)c2cc(-c4ccccc4)ccc2n3-c2c(-c3ccccc3)cc(-c3nc(-c4ccccc4)cc(-c4ccccc4)n3)cc2-c2ccccc2)cc1]

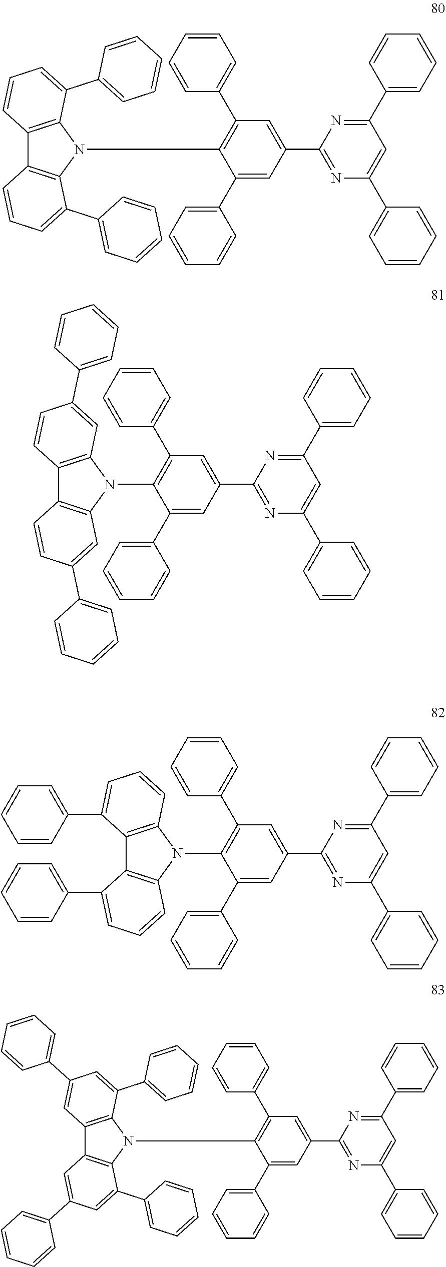 [SMILES: c1ccc(-c2cc(-c3ccccc3)nc(-c3cc(-c4ccccc4)c(-n4c5c(-c6ccccc6)cccc5c5cccc(-c6ccccc6)c54)c(-c4ccccc4)c3)n2)cc1 ; c1ccc(-c2ccc3c4ccc(-c5ccccc5)cc4n(-c4c(-c5ccccc5)cc(-c5nc(-c6ccccc6)cc(-c6ccccc6)n5)cc4-c4ccccc4)c3c2)cc1 ; c1ccc(-c2cc(-c3ccccc3)nc(-c3cc(-c4ccccc4)c(-n4c5cccc(-c6ccccc6)c5c5c(-c6ccccc6)cccc54)c(-c4ccccc4)c3)n2)cc1 ; c1ccc(-c2cc(-c3ccccc3)c3c(c2)c2cc(-c4ccccc4)cc(-c4ccccc4)c2n3-c2c(-c3ccccc3)cc(-c3nc(-c4ccccc4)cc(-c4ccccc4)n3)cc2-c2ccccc2)cc1]

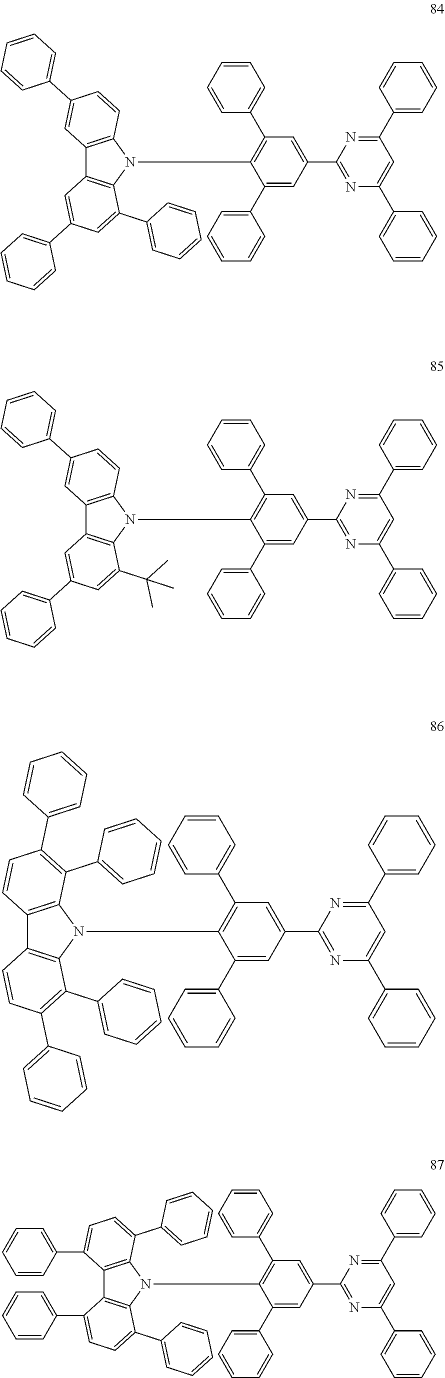 [SMILES: c1ccc(-c2ccc3c(c2)c2cc(-c4ccccc4)cc(-c4ccccc4)c2n3-c2c(-c3ccccc3)cc(-c3nc(-c4ccccc4)cc(-c4ccccc4)n3)cc2-c2ccccc2)cc1 ; CC(C)(C)c1cc(-c2ccccc2)cc2c3cc(-c4ccccc4)ccc3n(-c3c(-c4ccccc4)cc(-c4nc(-c5ccccc5)cc(-c5ccccc5)n4)cc3-c3ccccc3)c12 ; c1ccc(-c2cc(-c3ccccc3)nc(-c3cc(-c4ccccc4)c(-n4c5c(-c6ccccc6)c(-c6ccccc6)ccc5c5ccc(-c6ccccc6)c(-c6ccccc6)c54)c(-c4ccccc4)c3)n2)cc1 ; c1ccc(-c2cc(-c3ccccc3)nc(-c3cc(-c4ccccc4)c(-n4c5c(-c6ccccc6)ccc(-c6ccccc6)c5c5c(-c6ccccc6)ccc(-c6ccccc6)c54)c(-c4ccccc4)c3)n2)cc1]

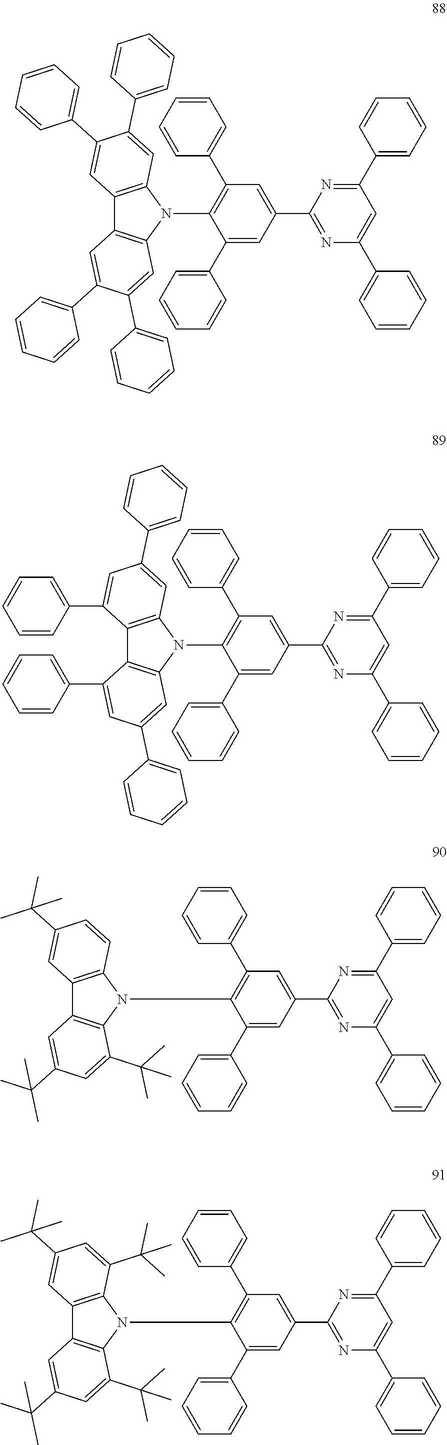 [SMILES: c1ccc(-c2cc(-c3ccccc3)nc(-c3cc(-c4ccccc4)c(-n4c5cc(-c6ccccc6)c(-c6ccccc6)cc5c5cc(-c6ccccc6)c(-c6ccccc6)cc54)c(-c4ccccc4)c3)n2)cc1 ; c1ccc(-c2cc(-c3ccccc3)c3c4c(-c5ccccc5)cc(-c5ccccc5)cc4n(-c4c(-c5ccccc5)cc(-c5nc(-c6ccccc6)cc(-c6ccccc6)n5)cc4-c4ccccc4)c3c2)cc1 ; CC(C)(C)c1ccc2c(c1)c1cc(C(C)(C)C)cc(C(C)(C)C)c1n2-c1c(-c2ccccc2)cc(-c2nc(-c3ccccc3)cc(-c3ccccc3)n2)cc1-c1ccccc1 ; CC(C)(C)c1cc(C(C)(C)C)c2c(c1)c1cc(C(C)(C)C)cc(C(C)(C)C)c1n2-c1c(-c2ccccc2)cc(-c2nc(-c3ccccc3)cc(-c3ccccc3)n2)cc1-c1ccccc1]

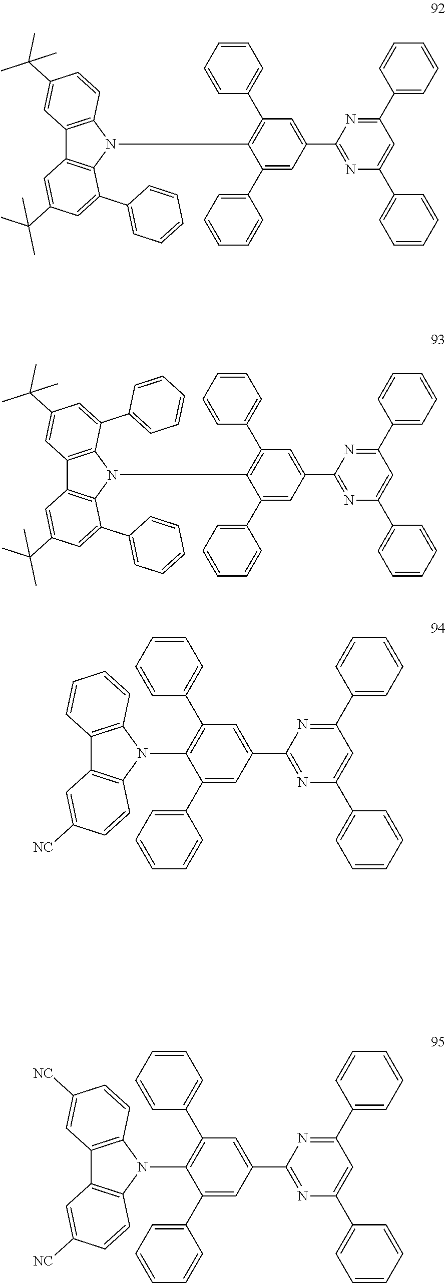 [SMILES: CC(C)(C)c1ccc2c(c1)c1cc(C(C)(C)C)cc(-c3ccccc3)c1n2-c1c(-c2ccccc2)cc(-c2nc(-c3ccccc3)cc(-c3ccccc3)n2)cc1-c1ccccc1 ; CC(C)(C)c1cc(-c2ccccc2)c2c(c1)c1cc(C(C)(C)C)cc(-c3ccccc3)c1n2-c1c(-c2ccccc2)cc(-c2nc(-c3ccccc3)cc(-c3ccccc3)n2)cc1-c1ccccc1 ; N#Cc1ccc2c(c1)c1ccccc1n2-c1c(-c2ccccc2)cc(-c2nc(-c3ccccc3)cc(-c3ccccc3)n2)cc1-c1ccccc1 ; N#Cc1ccc2c(c1)c1cc(C#N)ccc1n2-c1c(-c2ccccc2)cc(-c2nc(-c3ccccc3)cc(-c3ccccc3)n2)cc1-c1ccccc1]

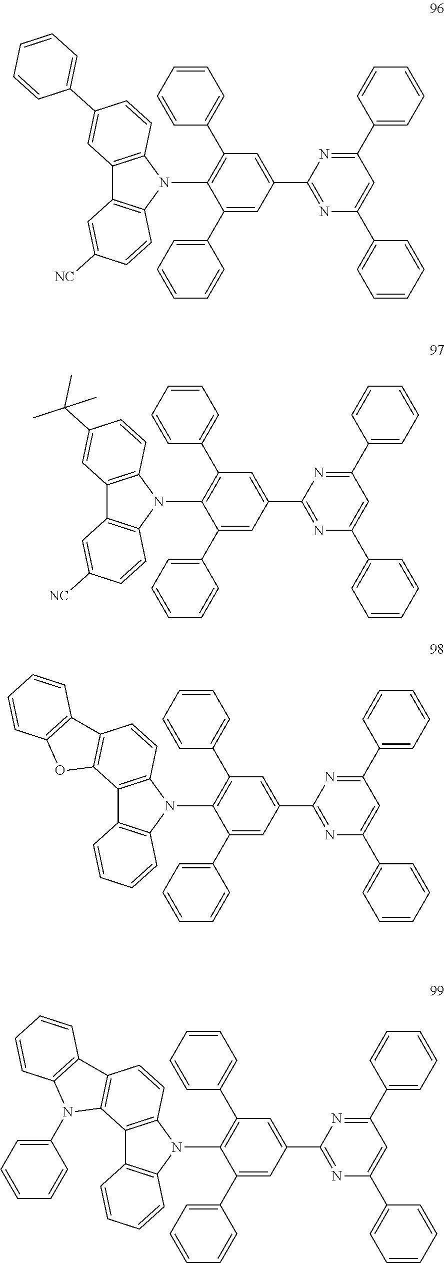 [SMILES: N#Cc1ccc2c(c1)c1cc(-c3ccccc3)ccc1n2-c1c(-c2ccccc2)cc(-c2nc(-c3ccccc3)cc(-c3ccccc3)n2)cc1-c1ccccc1 ; CC(C)(C)c1ccc2c(c1)c1cc(C#N)ccc1n2-c1c(-c2ccccc2)cc(-c2nc(-c3ccccc3)cc(-c3ccccc3)n2)cc1-c1ccccc1 ; c1ccc(-c2cc(-c3ccccc3)nc(-c3cc(-c4ccccc4)c(-n4c5ccccc5c5c6oc7ccccc7c6ccc54)c(-c4ccccc4)c3)n2)cc1 ; c1ccc(-c2cc(-c3ccccc3)nc(-c3cc(-c4ccccc4)c(-n4c5ccccc5c5c4ccc4c6ccccc6n(-c6ccccc6)c45)c(-c4ccccc4)c3)n2)cc1]

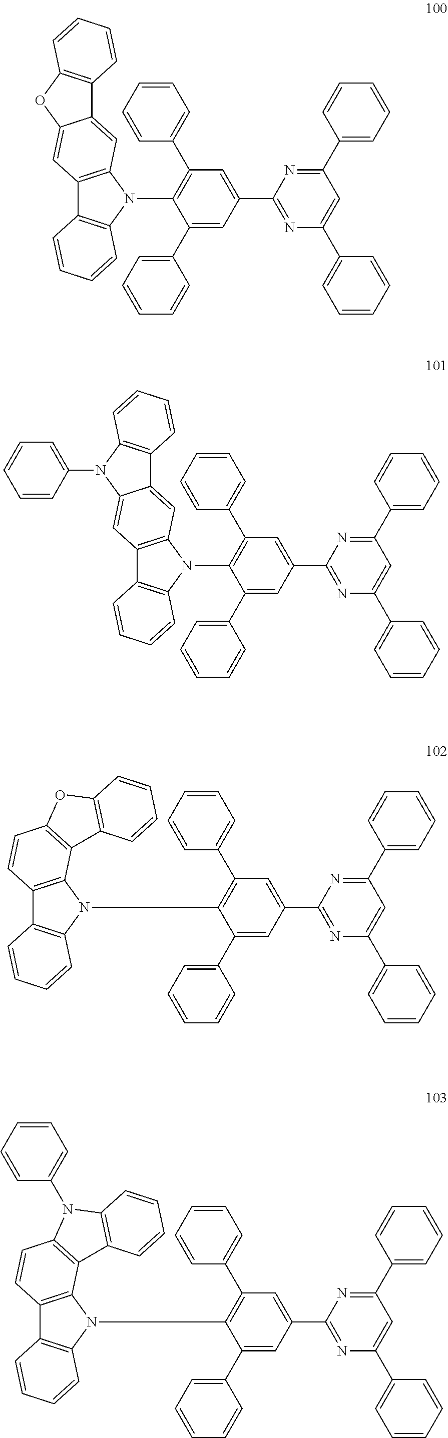 [SMILES: c1ccc(-c2cc(-c3ccccc3)nc(-c3cc(-c4ccccc4)c(-n4c5ccccc5c5cc6oc7ccccc7c6cc54)c(-c4ccccc4)c3)n2)cc1 ; c1ccc(-c2cc(-c3ccccc3)nc(-c3cc(-c4ccccc4)c(-n4c5ccccc5c5cc6c(cc54)c4ccccc4n6-c4ccccc4)c(-c4ccccc4)c3)n2)cc1 ; c1ccc(-c2cc(-c3ccccc3)nc(-c3cc(-c4ccccc4)c(-n4c5ccccc5c5ccc6oc7ccccc7c6c54)c(-c4ccccc4)c3)n2)cc1 ; c1ccc(-c2cc(-c3ccccc3)nc(-c3cc(-c4ccccc4)c(-n4c5ccccc5c5ccc6c(c7ccccc7n6-c6ccccc6)c54)c(-c4ccccc4)c3)n2)cc1]

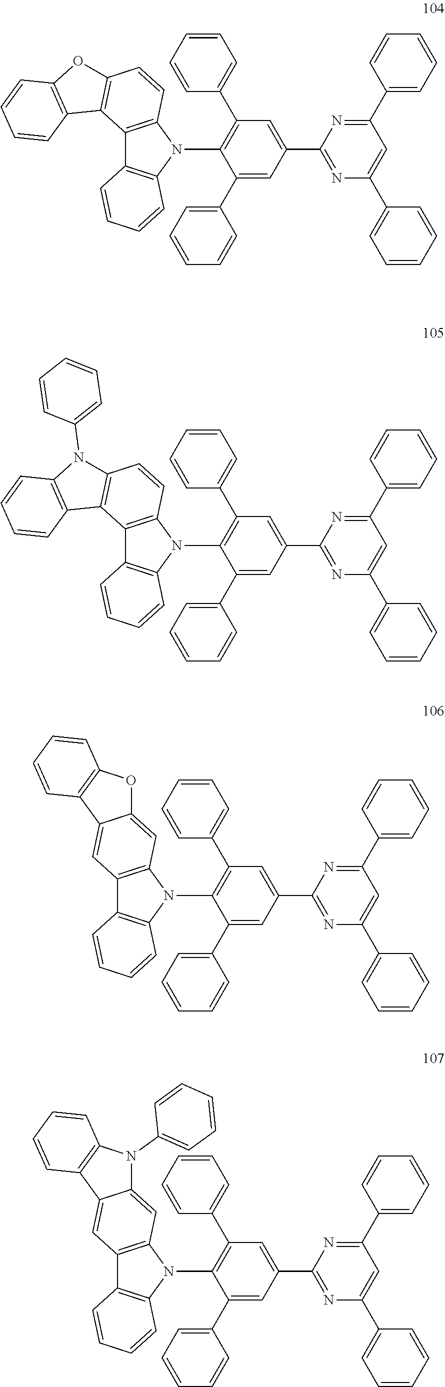 [SMILES: c1ccc(-c2cc(-c3ccccc3)nc(-c3cc(-c4ccccc4)c(-n4c5ccccc5c5c6c(ccc54)oc4ccccc46)c(-c4ccccc4)c3)n2)cc1 ; c1ccc(-c2cc(-c3ccccc3)nc(-c3cc(-c4ccccc4)c(-n4c5ccccc5c5c6c7ccccc7n(-c7ccccc7)c6ccc54)c(-c4ccccc4)c3)n2)cc1 ; c1ccc(-c2cc(-c3ccccc3)nc(-c3cc(-c4ccccc4)c(-n4c5ccccc5c5cc6c(cc54)oc4ccccc46)c(-c4ccccc4)c3)n2)cc1 ; c1ccc(-c2cc(-c3ccccc3)nc(-c3cc(-c4ccccc4)c(-n4c5ccccc5c5cc6c7ccccc7n(-c7ccccc7)c6cc54)c(-c4ccccc4)c3)n2)cc1]

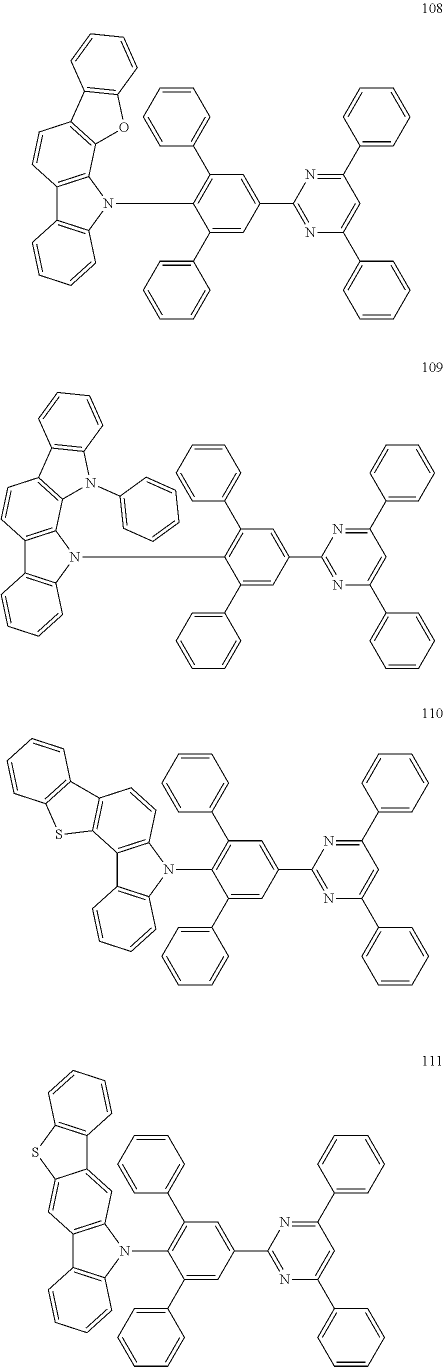 [SMILES: c1ccc(-c2cc(-c3ccccc3)nc(-c3cc(-c4ccccc4)c(-n4c5ccccc5c5ccc6c7ccccc7oc6c54)c(-c4ccccc4)c3)n2)cc1 ; c1ccc(-c2cc(-c3ccccc3)nc(-c3cc(-c4ccccc4)c(-n4c5ccccc5c5ccc6c7ccccc7n(-c7ccccc7)c6c54)c(-c4ccccc4)c3)n2)cc1 ; c1ccc(-c2cc(-c3ccccc3)nc(-c3cc(-c4ccccc4)c(-n4c5ccccc5c5c6sc7ccccc7c6ccc54)c(-c4ccccc4)c3)n2)cc1 ; c1ccc(-c2cc(-c3ccccc3)nc(-c3cc(-c4ccccc4)c(-n4c5ccccc5c5cc6sc7ccccc7c6cc54)c(-c4ccccc4)c3)n2)cc1]

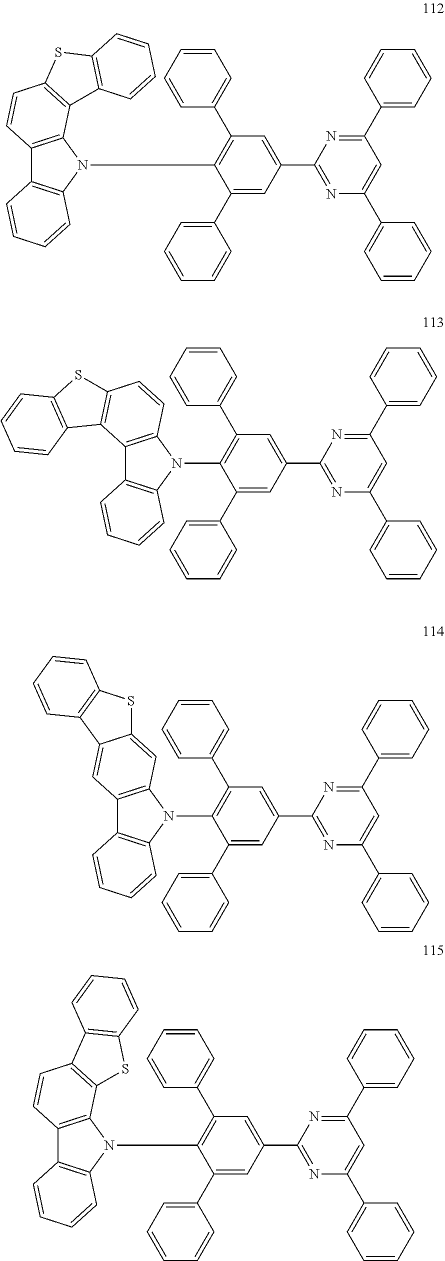 [SMILES: c1ccc(-c2cc(-c3ccccc3)nc(-c3cc(-c4ccccc4)c(-n4c5ccccc5c5ccc6sc7ccccc7c6c54)c(-c4ccccc4)c3)n2)cc1 ; c1ccc(-c2cc(-c3ccccc3)nc(-c3cc(-c4ccccc4)c(-n4c5ccccc5c5c6c(ccc54)sc4ccccc46)c(-c4ccccc4)c3)n2)cc1 ; c1ccc(-c2cc(-c3ccccc3)nc(-c3cc(-c4ccccc4)c(-n4c5ccccc5c5cc6c(cc54)sc4ccccc46)c(-c4ccccc4)c3)n2)cc1 ; c1ccc(-c2cc(-c3ccccc3)nc(-c3cc(-c4ccccc4)c(-n4c5ccccc5c5ccc6c7ccccc7sc6c54)c(-c4ccccc4)c3)n2)cc1]

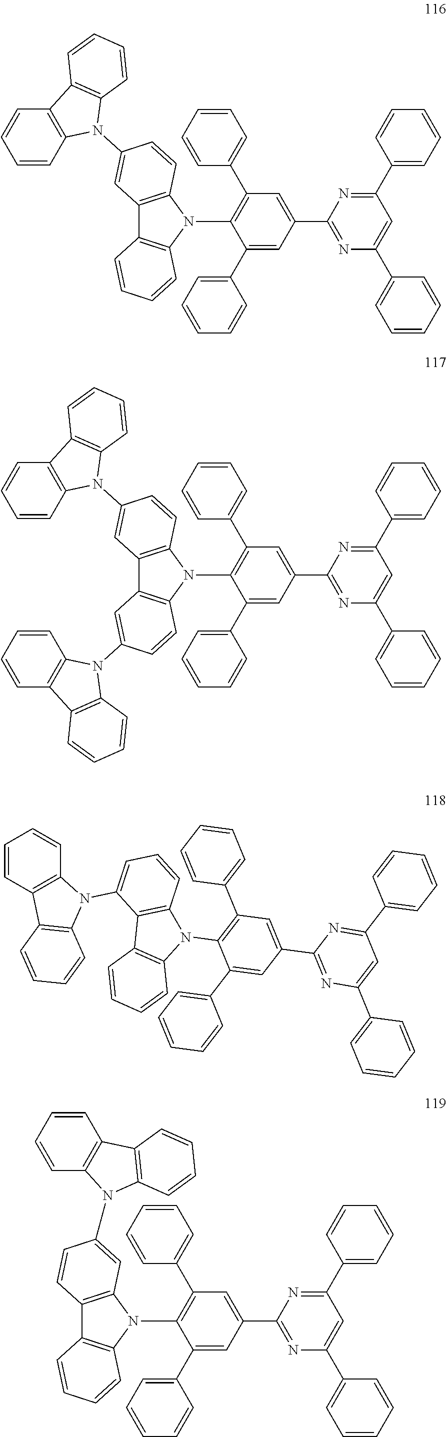 [SMILES: c1ccc(-c2cc(-c3ccccc3)nc(-c3cc(-c4ccccc4)c(-n4c5ccccc5c5cc(-n6c7ccccc7c7ccccc76)ccc54)c(-c4ccccc4)c3)n2)cc1 ; c1ccc(-c2cc(-c3ccccc3)nc(-c3cc(-c4ccccc4)c(-n4c5ccc(-n6c7ccccc7c7ccccc76)cc5c5cc(-n6c7ccccc7c7ccccc76)ccc54)c(-c4ccccc4)c3)n2)cc1 ; c1ccc(-c2cc(-c3ccccc3)nc(-c3cc(-c4ccccc4)c(-n4c5ccccc5c5c(-n6c7ccccc7c7ccccc76)cccc54)c(-c4ccccc4)c3)n2)cc1 ; c1ccc(-c2cc(-c3ccccc3)nc(-c3cc(-c4ccccc4)c(-n4c5ccccc5c5ccc(-n6c7ccccc7c7ccccc76)cc54)c(-c4ccccc4)c3)n2)cc1]

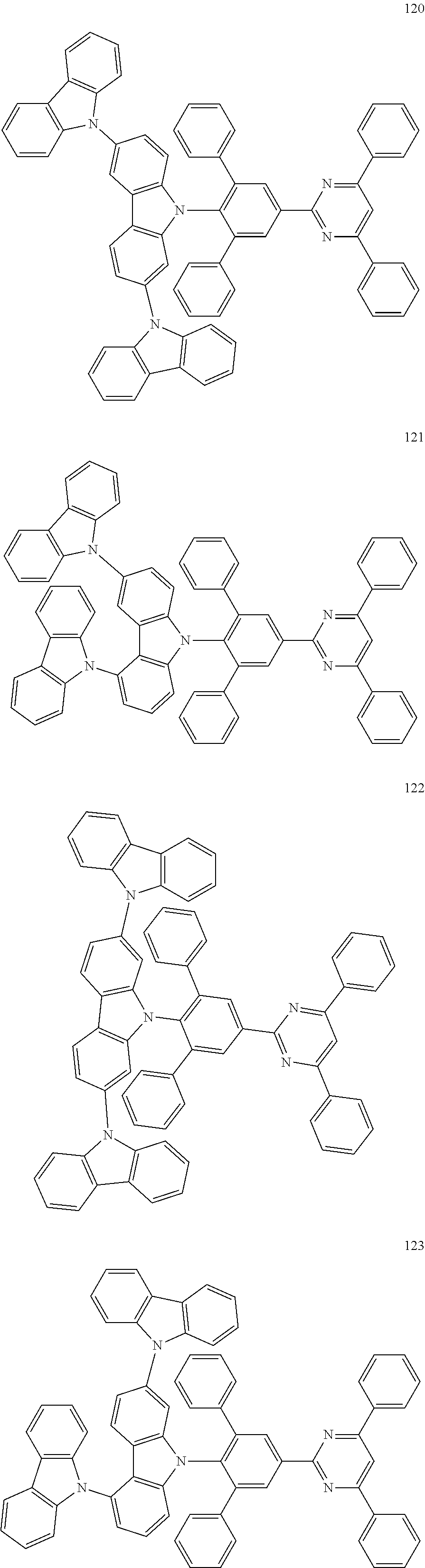

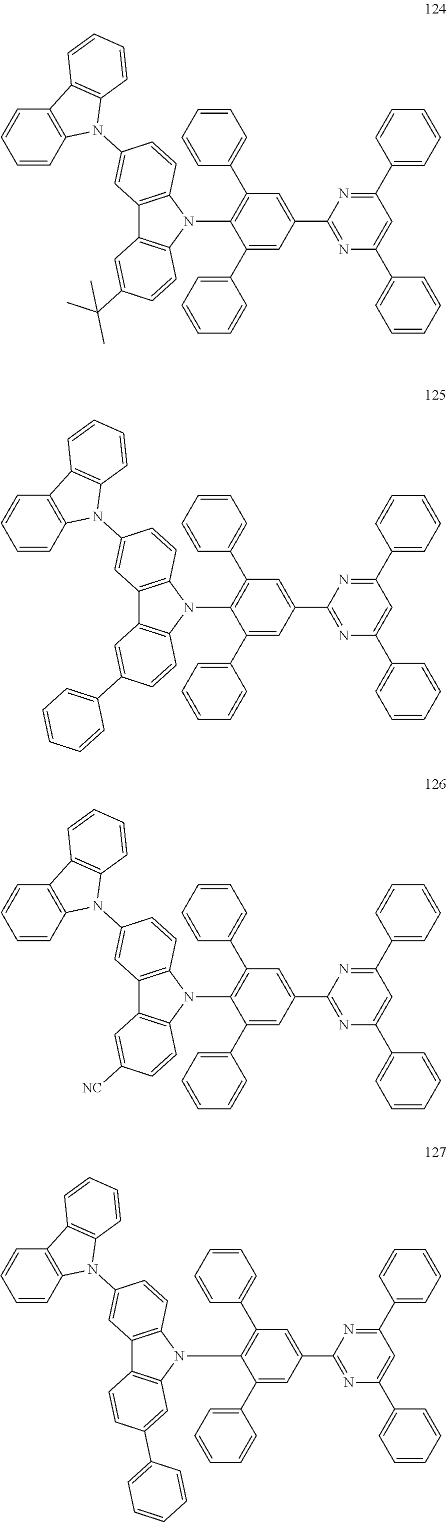 [SMILES: CC(C)(C)c1ccc2c(c1)c1cc(-n3c4ccccc4c4ccccc43)ccc1n2-c1c(-c2ccccc2)cc(-c2nc(-c3ccccc3)cc(-c3ccccc3)n2)cc1-c1ccccc1 ; c1ccc(-c2ccc3c(c2)c2cc(-n4c5ccccc5c5ccccc54)ccc2n3-c2c(-c3ccccc3)cc(-c3nc(-c4ccccc4)cc(-c4ccccc4)n3)cc2-c2ccccc2)cc1 ; N#Cc1ccc2c(c1)c1cc(-n3c4ccccc4c4ccccc43)ccc1n2-c1c(-c2ccccc2)cc(-c2nc(-c3ccccc3)cc(-c3ccccc3)n2)cc1-c1ccccc1 ; c1ccc(-c2ccc3c4cc(-n5c6ccccc6c6ccccc65)ccc4n(-c4c(-c5ccccc5)cc(-c5nc(-c6ccccc6)cc(-c6ccccc6)n5)cc4-c4ccccc4)c3c2)cc1]

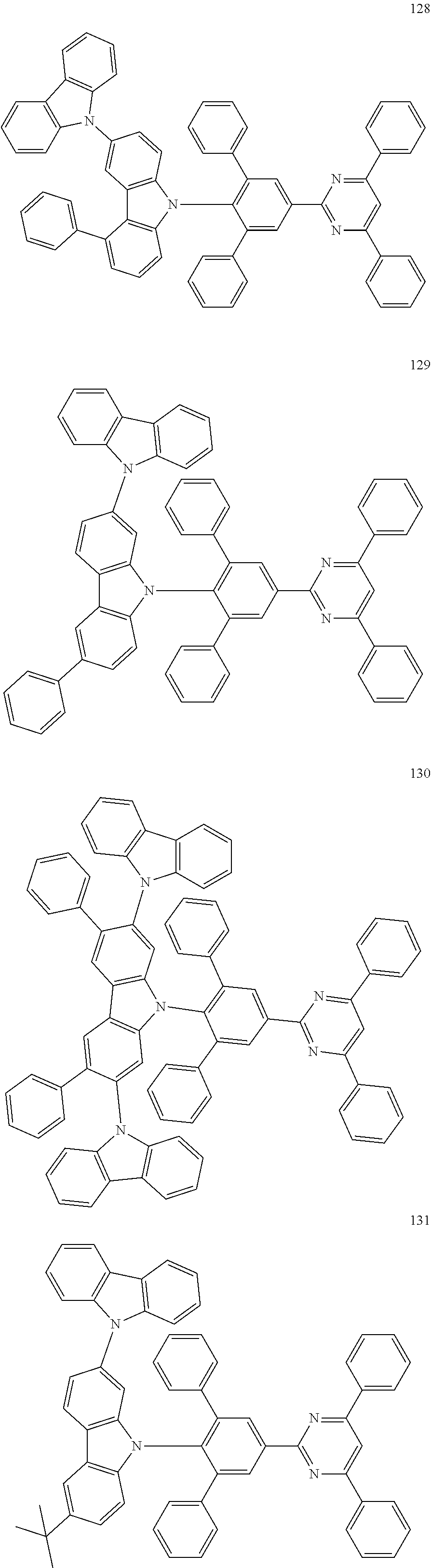

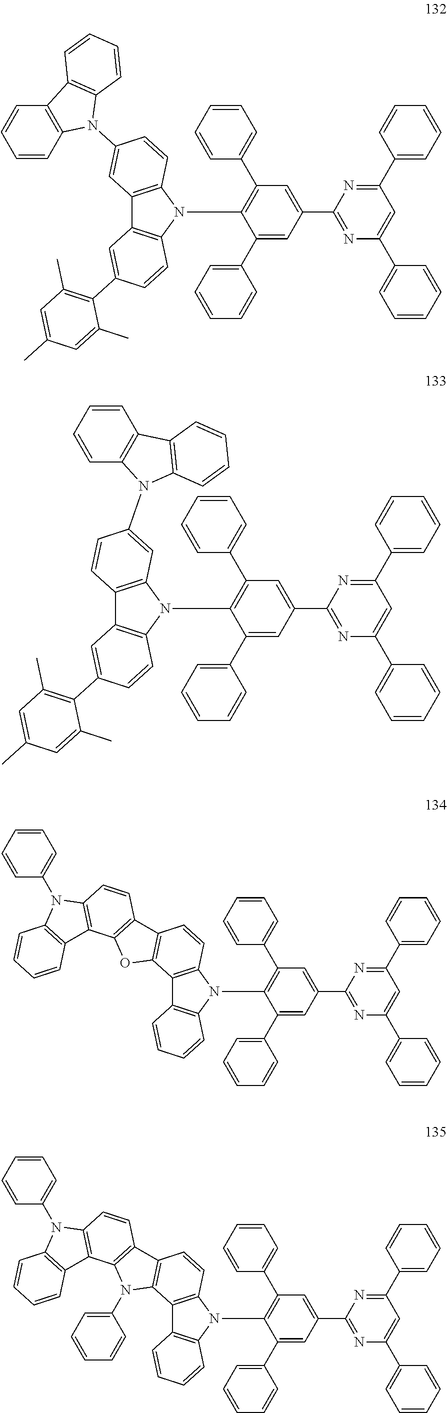

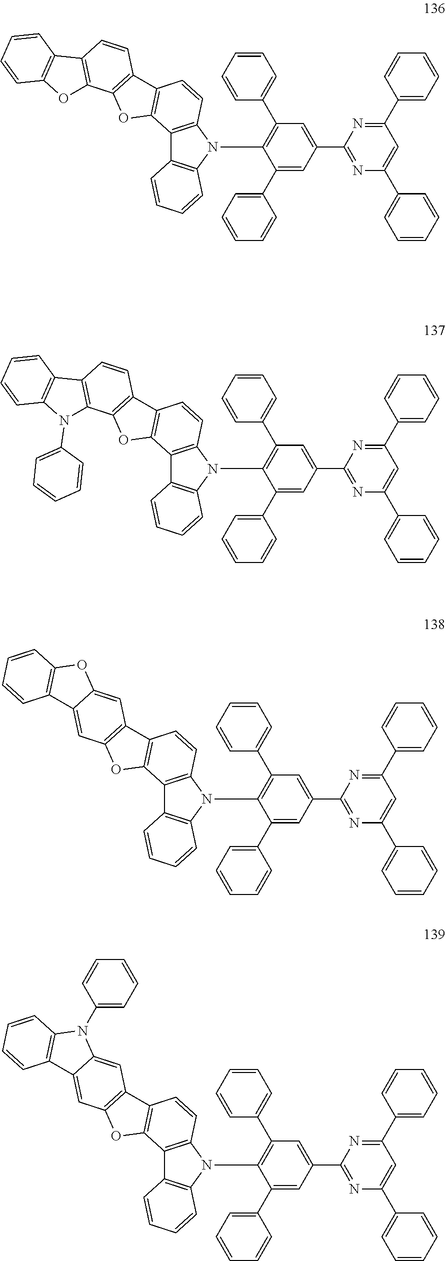 [SMILES: c1ccc(-c2cc(-c3ccccc3)nc(-c3cc(-c4ccccc4)c(-n4c5ccccc5c5c6oc7c(ccc8c9ccccc9oc87)c6ccc54)c(-c4ccccc4)c3)n2)cc1 ; c1ccc(-c2cc(-c3ccccc3)nc(-c3cc(-c4ccccc4)c(-n4c5ccccc5c5c6oc7c(ccc8c9ccccc9n(-c9ccccc9)c87)c6ccc54)c(-c4ccccc4)c3)n2)cc1 ; c1ccc(-c2cc(-c3ccccc3)nc(-c3cc(-c4ccccc4)c(-n4c5ccccc5c5c6oc7cc8c(cc7c6ccc54)oc4ccccc48)c(-c4ccccc4)c3)n2)cc1 ; c1ccc(-c2cc(-c3ccccc3)nc(-c3cc(-c4ccccc4)c(-n4c5ccccc5c5c6oc7cc8c9ccccc9n(-c9ccccc9)c8cc7c6ccc54)c(-c4ccccc4)c3)n2)cc1]

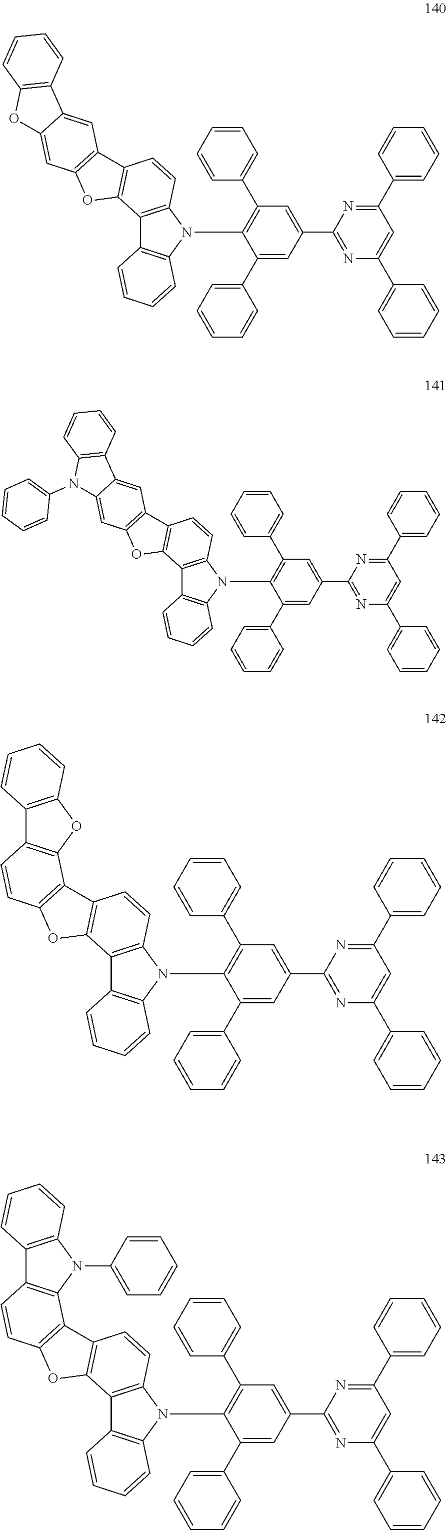 [SMILES: c1ccc(-c2cc(-c3ccccc3)nc(-c3cc(-c4ccccc4)c(-n4c5ccccc5c5c6oc7cc8oc9ccccc9c8cc7c6ccc54)c(-c4ccccc4)c3)n2)cc1 ; c1ccc(-c2cc(-c3ccccc3)nc(-c3cc(-c4ccccc4)c(-n4c5ccccc5c5c6oc7cc8c(cc7c6ccc54)c4ccccc4n8-c4ccccc4)c(-c4ccccc4)c3)n2)cc1 ; c1ccc(-c2cc(-c3ccccc3)nc(-c3cc(-c4ccccc4)c(-n4c5ccccc5c5c6oc7ccc8c9ccccc9oc8c7c6ccc54)c(-c4ccccc4)c3)n2)cc1 ; c1ccc(-c2cc(-c3ccccc3)nc(-c3cc(-c4ccccc4)c(-n4c5ccccc5c5c6oc7ccc8c9ccccc9n(-c9ccccc9)c8c7c6ccc54)c(-c4ccccc4)c3)n2)cc1]

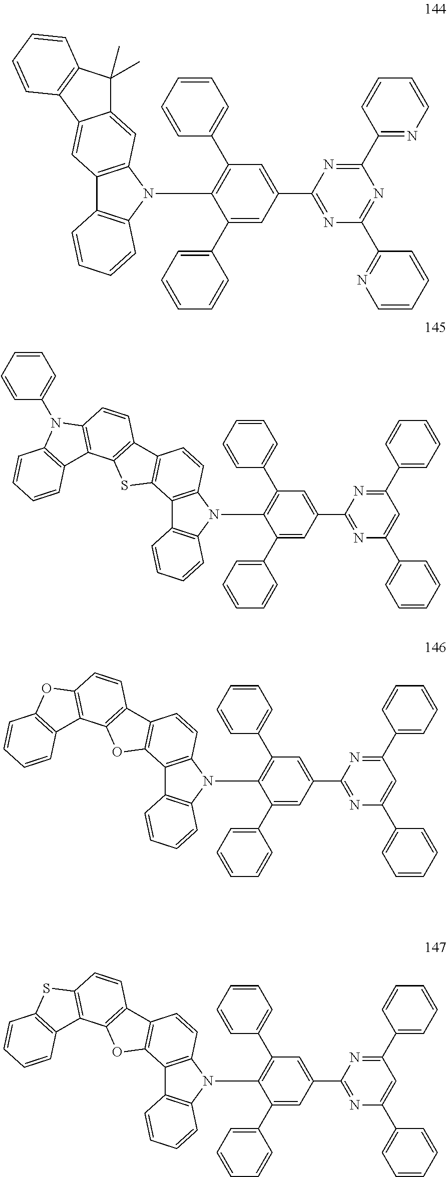 [SMILES: CC1(C)c2ccccc2-c2cc3c4ccccc4n(-c4c(-c5ccccc5)cc(-c5nc(-c6ccccn6)nc(-c6ccccn6)n5)cc4-c4ccccc4)c3cc21 ; c1ccc(-c2cc(-c3ccccc3)nc(-c3cc(-c4ccccc4)c(-n4c5ccccc5c5c6sc7c(ccc8c7c7ccccc7n8-c7ccccc7)c6ccc54)c(-c4ccccc4)c3)n2)cc1 ; c1ccc(-c2cc(-c3ccccc3)nc(-c3cc(-c4ccccc4)c(-n4c5ccccc5c5c6oc7c(ccc8oc9ccccc9c87)c6ccc54)c(-c4ccccc4)c3)n2)cc1 ; c1ccc(-c2cc(-c3ccccc3)nc(-c3cc(-c4ccccc4)c(-n4c5ccccc5c5c6oc7c(ccc8sc9ccccc9c87)c6ccc54)c(-c4ccccc4)c3)n2)cc1]

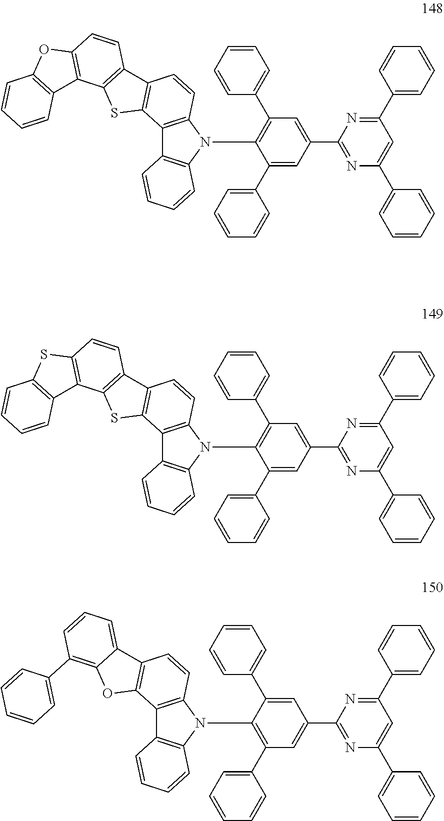 [SMILES: c1ccc(-c2cc(-c3ccccc3)nc(-c3cc(-c4ccccc4)c(-n4c5ccccc5c5c6sc7c(ccc8oc9ccccc9c87)c6ccc54)c(-c4ccccc4)c3)n2)cc1 ; c1ccc(-c2cc(-c3ccccc3)nc(-c3cc(-c4ccccc4)c(-n4c5ccccc5c5c6sc7c(ccc8sc9ccccc9c87)c6ccc54)c(-c4ccccc4)c3)n2)cc1 ; c1ccc(-c2cc(-c3ccccc3)nc(-c3cc(-c4ccccc4)c(-n4c5ccccc5c5c6oc7c(-c8ccccc8)cccc7c6ccc54)c(-c4ccccc4)c3)n2)cc1]

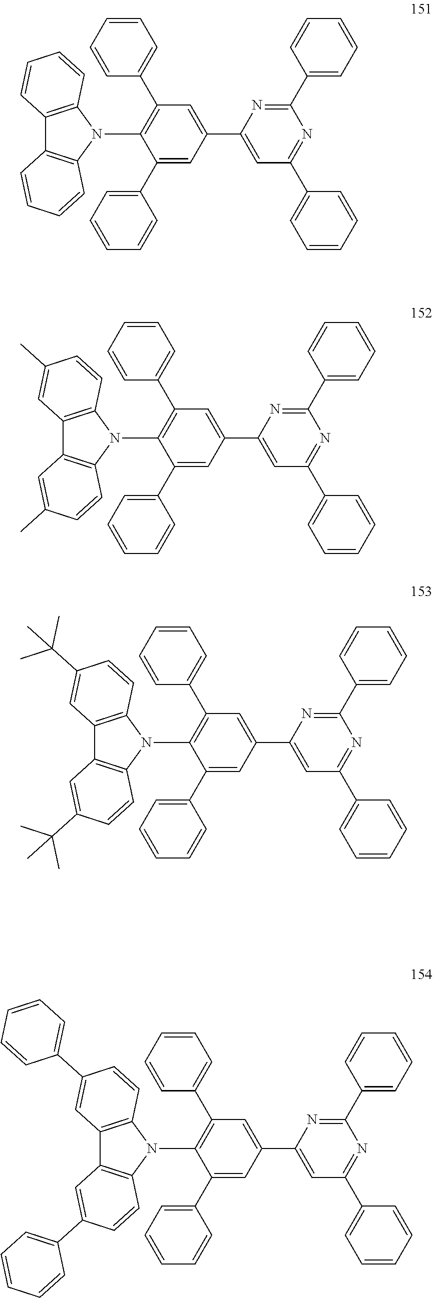 [SMILES: c1ccc(-c2cc(-c3cc(-c4ccccc4)c(-n4c5ccccc5c5ccccc54)c(-c4ccccc4)c3)nc(-c3ccccc3)n2)cc1 ; Cc1ccc2c(c1)c1cc(C)ccc1n2-c1c(-c2ccccc2)cc(-c2cc(-c3ccccc3)nc(-c3ccccc3)n2)cc1-c1ccccc1 ; CC(C)(C)c1ccc2c(c1)c1cc(C(C)(C)C)ccc1n2-c1c(-c2ccccc2)cc(-c2cc(-c3ccccc3)nc(-c3ccccc3)n2)cc1-c1ccccc1 ; c1ccc(-c2ccc3c(c2)c2cc(-c4ccccc4)ccc2n3-c2c(-c3ccccc3)cc(-c3cc(-c4ccccc4)nc(-c4ccccc4)n3)cc2-c2ccccc2)cc1]

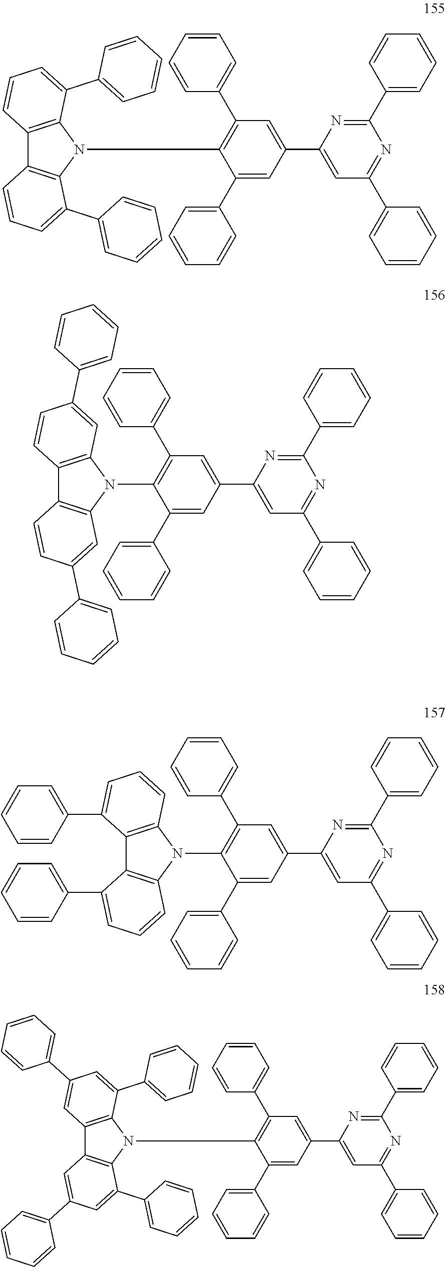 [SMILES: c1ccc(-c2cc(-c3cc(-c4ccccc4)c(-n4c5c(-c6ccccc6)cccc5c5cccc(-c6ccccc6)c54)c(-c4ccccc4)c3)nc(-c3ccccc3)n2)cc1 ; c1ccc(-c2ccc3c4ccc(-c5ccccc5)cc4n(-c4c(-c5ccccc5)cc(-c5cc(-c6ccccc6)nc(-c6ccccc6)n5)cc4-c4ccccc4)c3c2)cc1 ; c1ccc(-c2cc(-c3cc(-c4ccccc4)c(-n4c5cccc(-c6ccccc6)c5c5c(-c6ccccc6)cccc54)c(-c4ccccc4)c3)nc(-c3ccccc3)n2)cc1 ; c1ccc(-c2cc(-c3ccccc3)c3c(c2)c2cc(-c4ccccc4)cc(-c4ccccc4)c2n3-c2c(-c3ccccc3)cc(-c3cc(-c4ccccc4)nc(-c4ccccc4)n3)cc2-c2ccccc2)cc1]

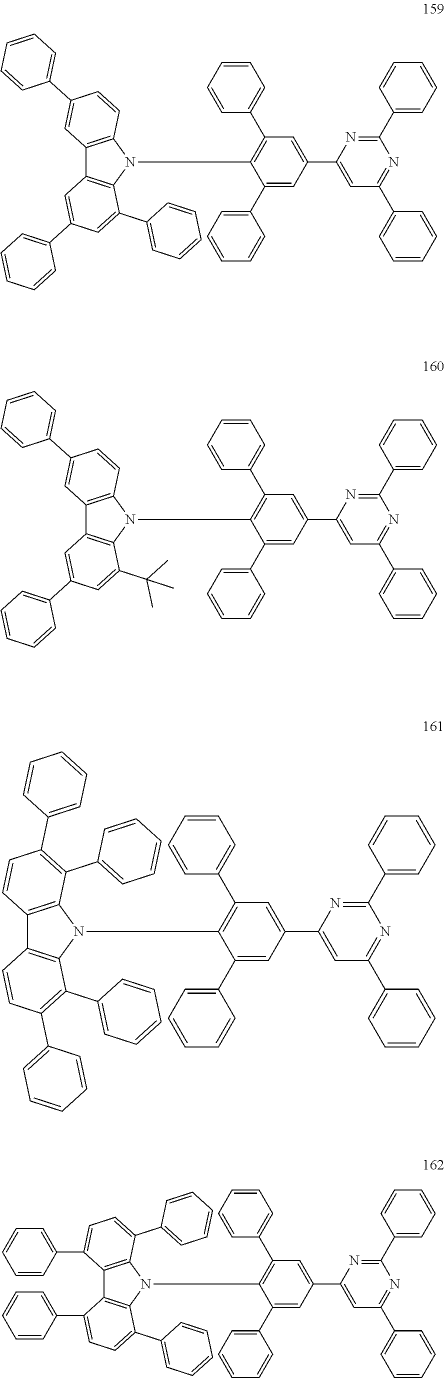 [SMILES: c1ccc(-c2ccc3c(c2)c2cc(-c4ccccc4)cc(-c4ccccc4)c2n3-c2c(-c3ccccc3)cc(-c3cc(-c4ccccc4)nc(-c4ccccc4)n3)cc2-c2ccccc2)cc1 ; CC(C)(C)c1cc(-c2ccccc2)cc2c3cc(-c4ccccc4)ccc3n(-c3c(-c4ccccc4)cc(-c4cc(-c5ccccc5)nc(-c5ccccc5)n4)cc3-c3ccccc3)c12 ; c1ccc(-c2cc(-c3cc(-c4ccccc4)c(-n4c5c(-c6ccccc6)c(-c6ccccc6)ccc5c5ccc(-c6ccccc6)c(-c6ccccc6)c54)c(-c4ccccc4)c3)nc(-c3ccccc3)n2)cc1 ; c1ccc(-c2cc(-c3cc(-c4ccccc4)c(-n4c5c(-c6ccccc6)ccc(-c6ccccc6)c5c5c(-c6ccccc6)ccc(-c6ccccc6)c54)c(-c4ccccc4)c3)nc(-c3ccccc3)n2)cc1]

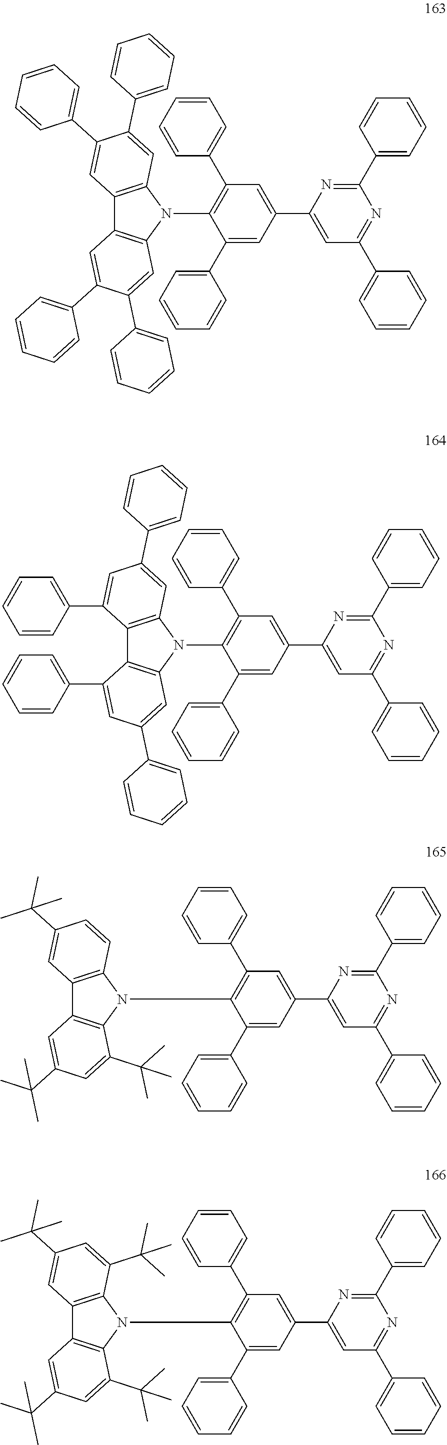 [SMILES: c1ccc(-c2cc(-c3cc(-c4ccccc4)c(-n4c5cc(-c6ccccc6)c(-c6ccccc6)cc5c5cc(-c6ccccc6)c(-c6ccccc6)cc54)c(-c4ccccc4)c3)nc(-c3ccccc3)n2)cc1 ; c1ccc(-c2cc(-c3ccccc3)c3c4c(-c5ccccc5)cc(-c5ccccc5)cc4n(-c4c(-c5ccccc5)cc(-c5cc(-c6ccccc6)nc(-c6ccccc6)n5)cc4-c4ccccc4)c3c2)cc1 ; CC(C)(C)c1ccc2c(c1)c1cc(C(C)(C)C)cc(C(C)(C)C)c1n2-c1c(-c2ccccc2)cc(-c2cc(-c3ccccc3)nc(-c3ccccc3)n2)cc1-c1ccccc1 ; CC(C)(C)c1cc(C(C)(C)C)c2c(c1)c1cc(C(C)(C)C)cc(C(C)(C)C)c1n2-c1c(-c2ccccc2)cc(-c2cc(-c3ccccc3)nc(-c3ccccc3)n2)cc1-c1ccccc1]

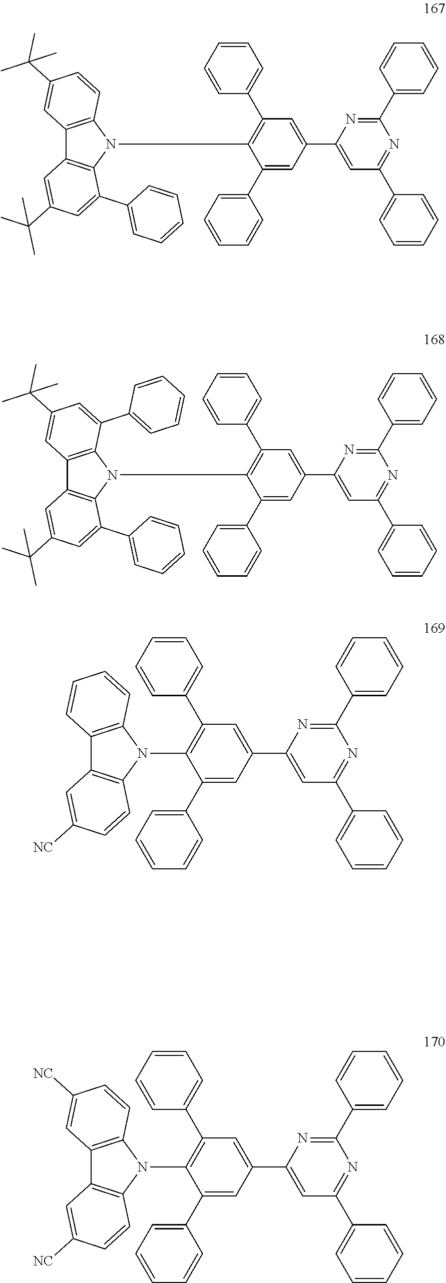 [SMILES: CC(C)(C)c1ccc2c(c1)c1cc(C(C)(C)C)cc(-c3ccccc3)c1n2-c1c(-c2ccccc2)cc(-c2cc(-c3ccccc3)nc(-c3ccccc3)n2)cc1-c1ccccc1 ; CC(C)(C)c1cc(-c2ccccc2)c2c(c1)c1cc(C(C)(C)C)cc(-c3ccccc3)c1n2-c1c(-c2ccccc2)cc(-c2cc(-c3ccccc3)nc(-c3ccccc3)n2)cc1-c1ccccc1 ; N#Cc1ccc2c(c1)c1ccccc1n2-c1c(-c2ccccc2)cc(-c2cc(-c3ccccc3)nc(-c3ccccc3)n2)cc1-c1ccccc1 ; N#Cc1ccc2c(c1)c1cc(C#N)ccc1n2-c1c(-c2ccccc2)cc(-c2cc(-c3ccccc3)nc(-c3ccccc3)n2)cc1-c1ccccc1]

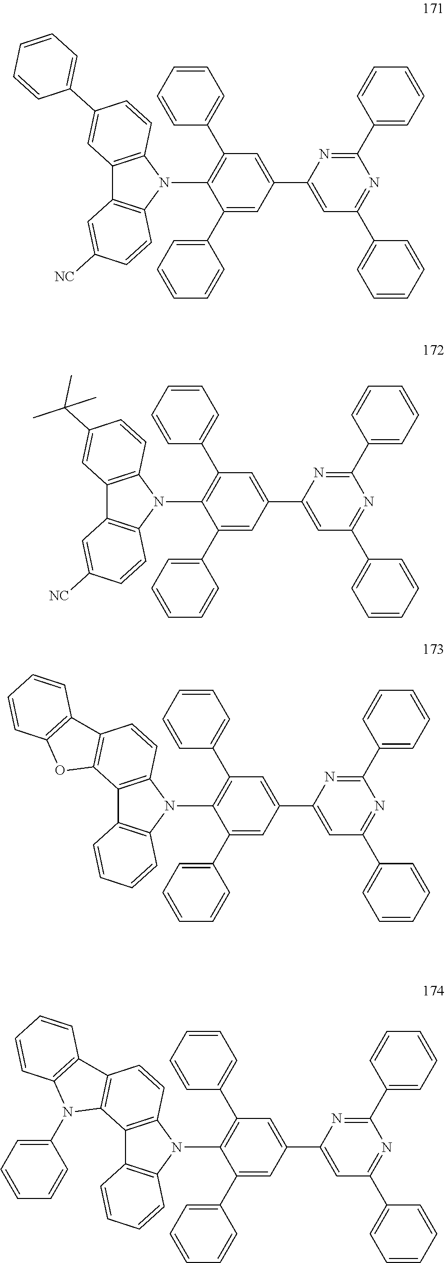 [SMILES: N#Cc1ccc2c(c1)c1cc(-c3ccccc3)ccc1n2-c1c(-c2ccccc2)cc(-c2cc(-c3ccccc3)nc(-c3ccccc3)n2)cc1-c1ccccc1 ; CC(C)(C)c1ccc2c(c1)c1cc(C#N)ccc1n2-c1c(-c2ccccc2)cc(-c2cc(-c3ccccc3)nc(-c3ccccc3)n2)cc1-c1ccccc1 ; c1ccc(-c2cc(-c3cc(-c4ccccc4)c(-n4c5ccccc5c5c6oc7ccccc7c6ccc54)c(-c4ccccc4)c3)nc(-c3ccccc3)n2)cc1 ; c1ccc(-c2cc(-c3cc(-c4ccccc4)c(-n4c5ccccc5c5c4ccc4c6ccccc6n(-c6ccccc6)c45)c(-c4ccccc4)c3)nc(-c3ccccc3)n2)cc1]

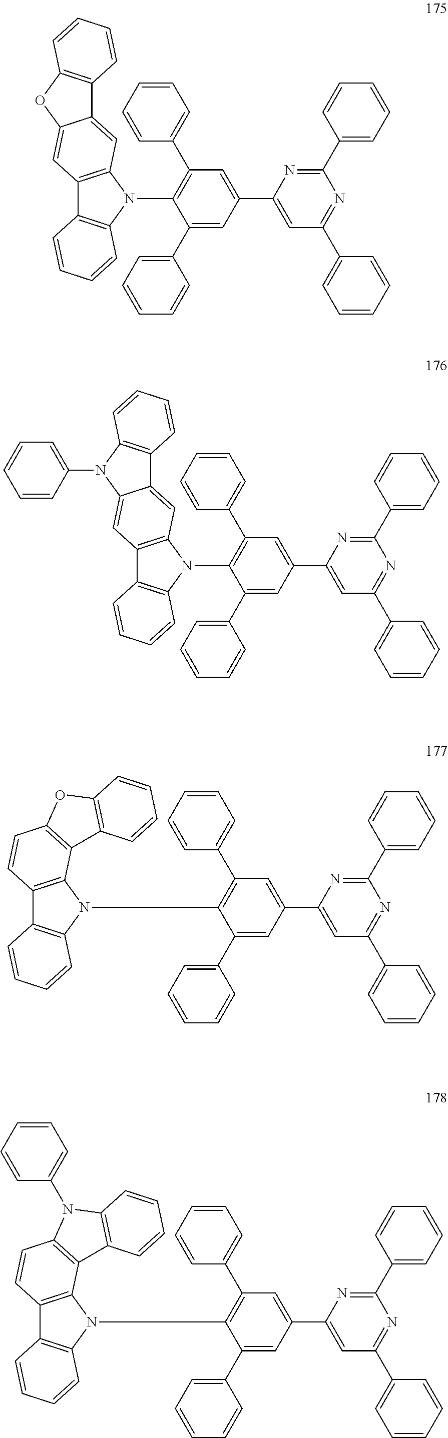 [SMILES: c1ccc(-c2cc(-c3cc(-c4ccccc4)c(-n4c5ccccc5c5cc6oc7ccccc7c6cc54)c(-c4ccccc4)c3)nc(-c3ccccc3)n2)cc1 ; c1ccc(-c2cc(-c3cc(-c4ccccc4)c(-n4c5ccccc5c5cc6c(cc54)c4ccccc4n6-c4ccccc4)c(-c4ccccc4)c3)nc(-c3ccccc3)n2)cc1 ; c1ccc(-c2cc(-c3cc(-c4ccccc4)c(-n4c5ccccc5c5ccc6oc7ccccc7c6c54)c(-c4ccccc4)c3)nc(-c3ccccc3)n2)cc1 ; c1ccc(-c2cc(-c3cc(-c4ccccc4)c(-n4c5ccccc5c5ccc6c(c7ccccc7n6-c6ccccc6)c54)c(-c4ccccc4)c3)nc(-c3ccccc3)n2)cc1]

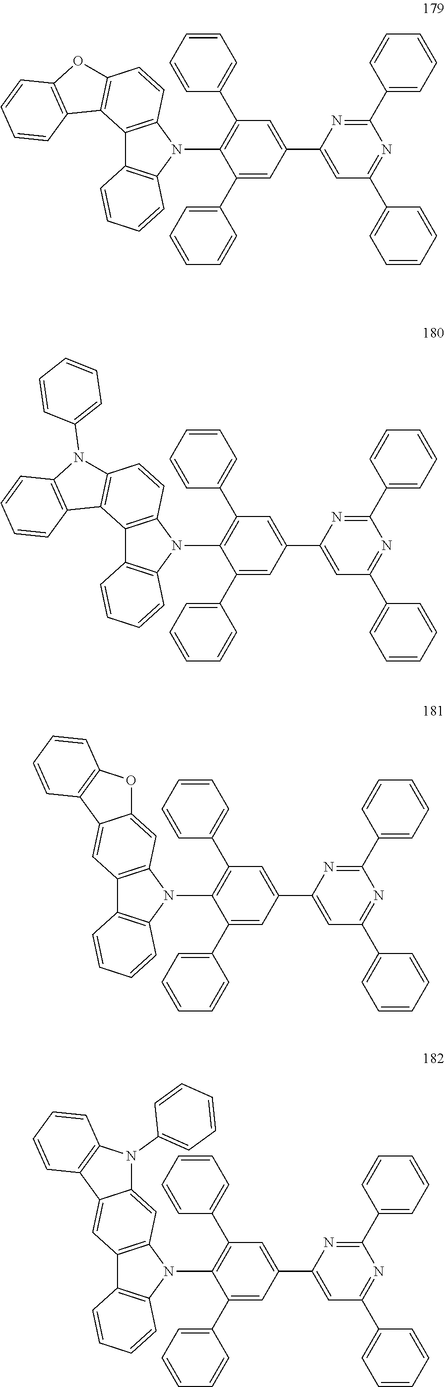 [SMILES: c1ccc(-c2cc(-c3cc(-c4ccccc4)c(-n4c5ccccc5c5c6c(ccc54)oc4ccccc46)c(-c4ccccc4)c3)nc(-c3ccccc3)n2)cc1 ; c1ccc(-c2cc(-c3cc(-c4ccccc4)c(-n4c5ccccc5c5c6c7ccccc7n(-c7ccccc7)c6ccc54)c(-c4ccccc4)c3)nc(-c3ccccc3)n2)cc1 ; c1ccc(-c2cc(-c3cc(-c4ccccc4)c(-n4c5ccccc5c5cc6c(cc54)oc4ccccc46)c(-c4ccccc4)c3)nc(-c3ccccc3)n2)cc1 ; c1ccc(-c2cc(-c3cc(-c4ccccc4)c(-n4c5ccccc5c5cc6c7ccccc7n(-c7ccccc7)c6cc54)c(-c4ccccc4)c3)nc(-c3ccccc3)n2)cc1]

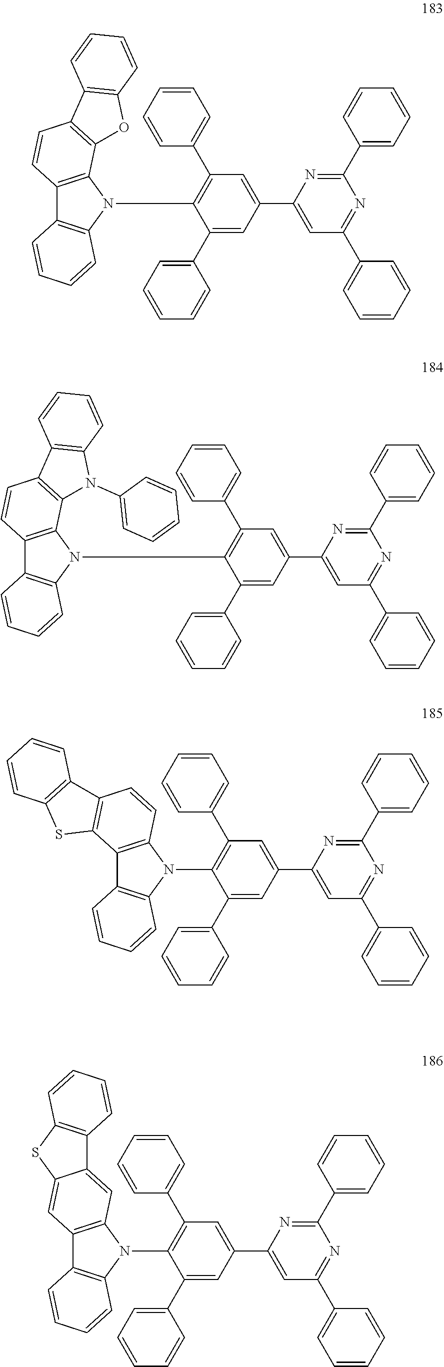 [SMILES: c1ccc(-c2cc(-c3cc(-c4ccccc4)c(-n4c5ccccc5c5ccc6c7ccccc7oc6c54)c(-c4ccccc4)c3)nc(-c3ccccc3)n2)cc1 ; c1ccc(-c2cc(-c3cc(-c4ccccc4)c(-n4c5ccccc5c5ccc6c7ccccc7n(-c7ccccc7)c6c54)c(-c4ccccc4)c3)nc(-c3ccccc3)n2)cc1 ; c1ccc(-c2cc(-c3cc(-c4ccccc4)c(-n4c5ccccc5c5c6sc7ccccc7c6ccc54)c(-c4ccccc4)c3)nc(-c3ccccc3)n2)cc1 ; c1ccc(-c2cc(-c3cc(-c4ccccc4)c(-n4c5ccccc5c5cc6sc7ccccc7c6cc54)c(-c4ccccc4)c3)nc(-c3ccccc3)n2)cc1]

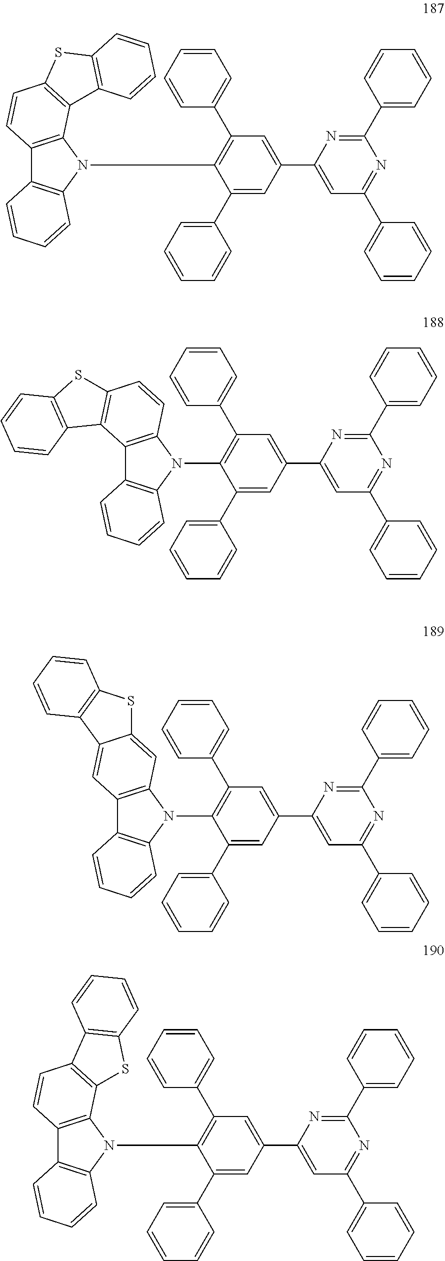 [SMILES: c1ccc(-c2cc(-c3cc(-c4ccccc4)c(-n4c5ccccc5c5ccc6sc7ccccc7c6c54)c(-c4ccccc4)c3)nc(-c3ccccc3)n2)cc1 ; c1ccc(-c2cc(-c3cc(-c4ccccc4)c(-n4c5ccccc5c5c6c(ccc54)sc4ccccc46)c(-c4ccccc4)c3)nc(-c3ccccc3)n2)cc1 ; c1ccc(-c2cc(-c3cc(-c4ccccc4)c(-n4c5ccccc5c5cc6c(cc54)sc4ccccc46)c(-c4ccccc4)c3)nc(-c3ccccc3)n2)cc1 ; c1ccc(-c2cc(-c3cc(-c4ccccc4)c(-n4c5ccccc5c5ccc6c7ccccc7sc6c54)c(-c4ccccc4)c3)nc(-c3ccccc3)n2)cc1]

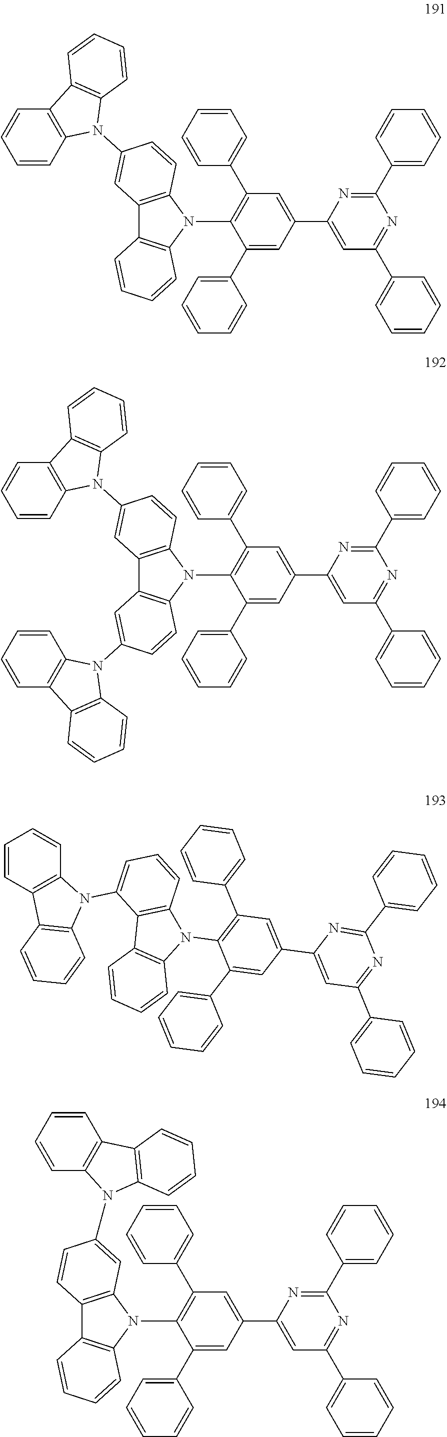 [SMILES: c1ccc(-c2cc(-c3cc(-c4ccccc4)c(-n4c5ccccc5c5cc(-n6c7ccccc7c7ccccc76)ccc54)c(-c4ccccc4)c3)nc(-c3ccccc3)n2)cc1 ; c1ccc(-c2cc(-c3cc(-c4ccccc4)c(-n4c5ccc(-n6c7ccccc7c7ccccc76)cc5c5cc(-n6c7ccccc7c7ccccc76)ccc54)c(-c4ccccc4)c3)nc(-c3ccccc3)n2)cc1 ; c1ccc(-c2cc(-c3cc(-c4ccccc4)c(-n4c5ccccc5c5c(-n6c7ccccc7c7ccccc76)cccc54)c(-c4ccccc4)c3)nc(-c3ccccc3)n2)cc1 ; c1ccc(-c2cc(-c3cc(-c4ccccc4)c(-n4c5ccccc5c5ccc(-n6c7ccccc7c7ccccc76)cc54)c(-c4ccccc4)c3)nc(-c3ccccc3)n2)cc1]

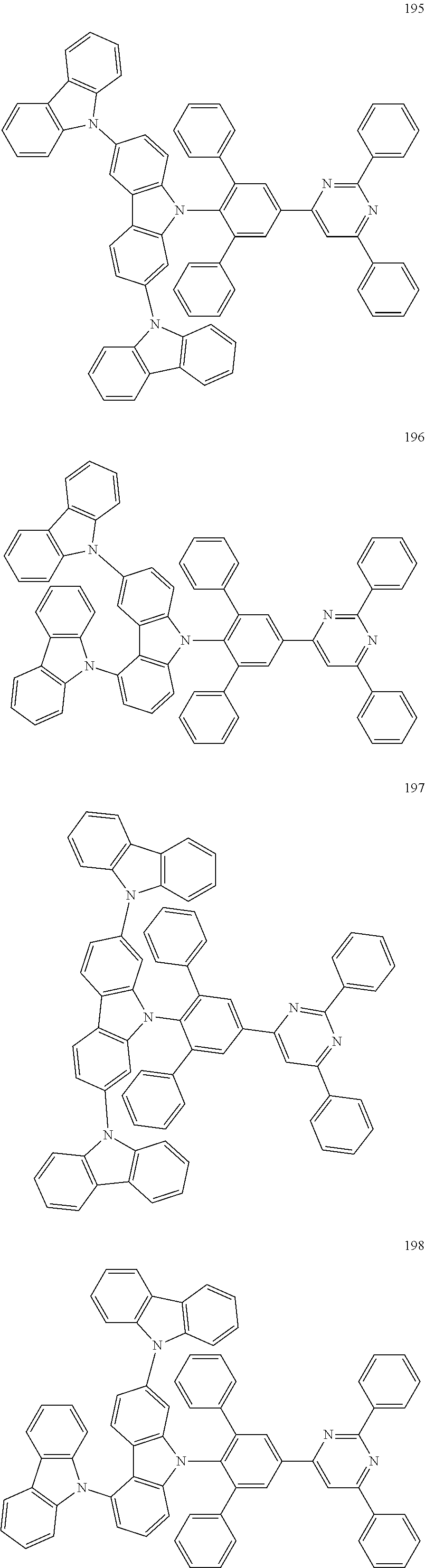

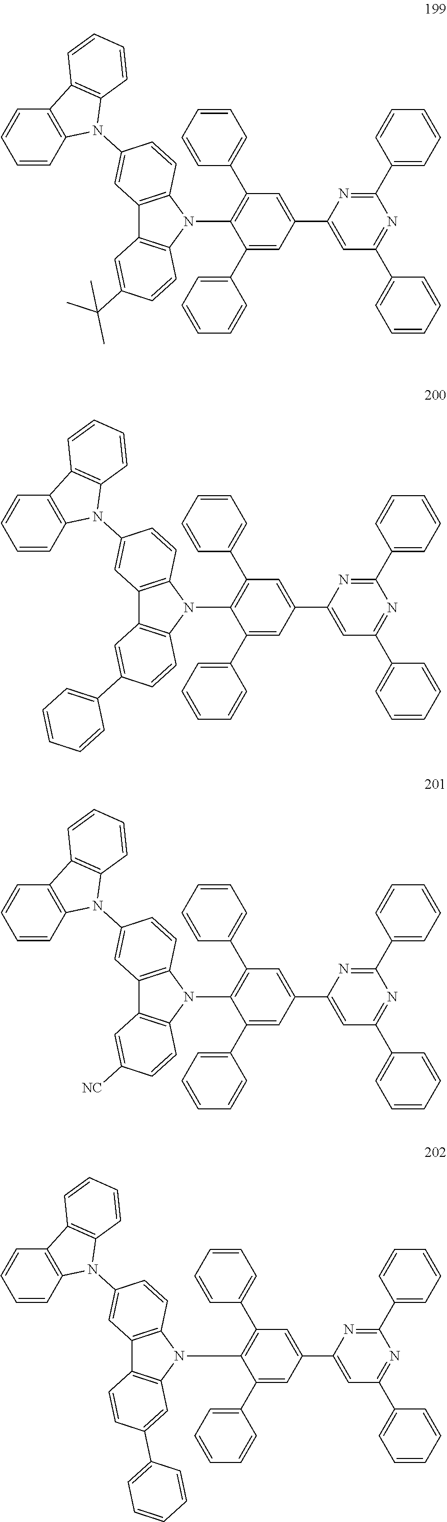 [SMILES: CC(C)(C)c1ccc2c(c1)c1cc(-n3c4ccccc4c4ccccc43)ccc1n2-c1c(-c2ccccc2)cc(-c2cc(-c3ccccc3)nc(-c3ccccc3)n2)cc1-c1ccccc1 ; c1ccc(-c2ccc3c(c2)c2cc(-n4c5ccccc5c5ccccc54)ccc2n3-c2c(-c3ccccc3)cc(-c3cc(-c4ccccc4)nc(-c4ccccc4)n3)cc2-c2ccccc2)cc1 ; N#Cc1ccc2c(c1)c1cc(-n3c4ccccc4c4ccccc43)ccc1n2-c1c(-c2ccccc2)cc(-c2cc(-c3ccccc3)nc(-c3ccccc3)n2)cc1-c1ccccc1 ; c1ccc(-c2ccc3c4cc(-n5c6ccccc6c6ccccc65)ccc4n(-c4c(-c5ccccc5)cc(-c5cc(-c6ccccc6)nc(-c6ccccc6)n5)cc4-c4ccccc4)c3c2)cc1]

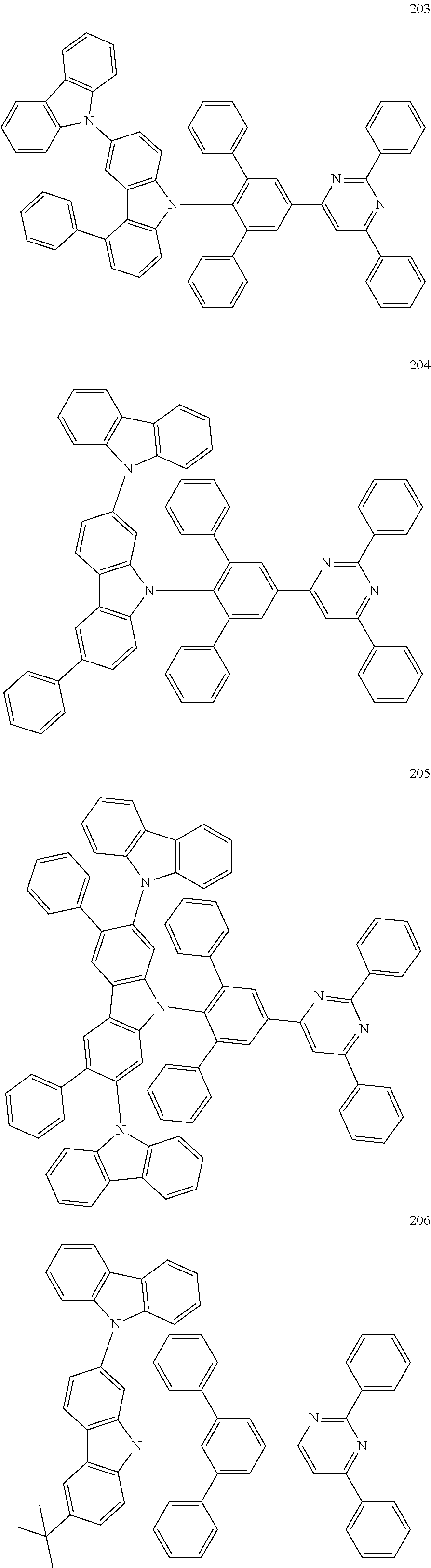

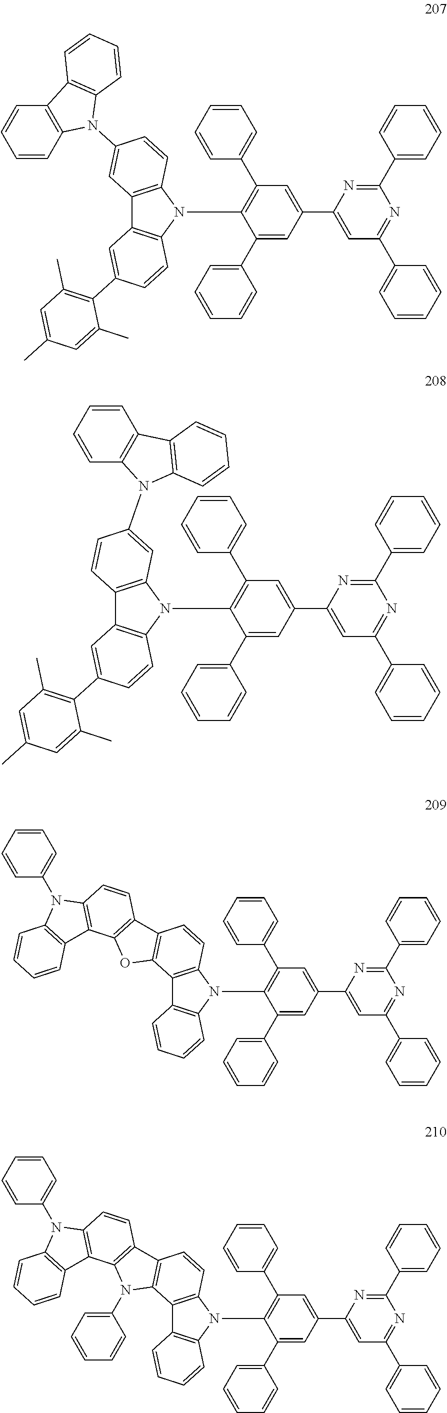

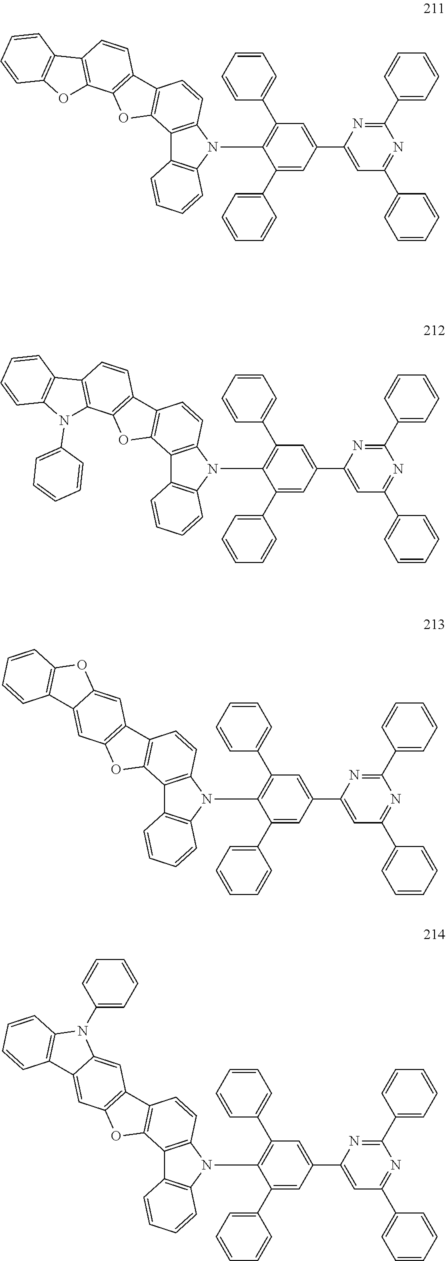 [SMILES: c1ccc(-c2cc(-c3cc(-c4ccccc4)c(-n4c5ccccc5c5c6oc7c(ccc8c9ccccc9oc87)c6ccc54)c(-c4ccccc4)c3)nc(-c3ccccc3)n2)cc1 ; c1ccc(-c2cc(-c3cc(-c4ccccc4)c(-n4c5ccccc5c5c6oc7c(ccc8c9ccccc9n(-c9ccccc9)c87)c6ccc54)c(-c4ccccc4)c3)nc(-c3ccccc3)n2)cc1 ; c1ccc(-c2cc(-c3cc(-c4ccccc4)c(-n4c5ccccc5c5c6oc7cc8c(cc7c6ccc54)oc4ccccc48)c(-c4ccccc4)c3)nc(-c3ccccc3)n2)cc1 ; c1ccc(-c2cc(-c3cc(-c4ccccc4)c(-n4c5ccccc5c5c6oc7cc8c9ccccc9n(-c9ccccc9)c8cc7c6ccc54)c(-c4ccccc4)c3)nc(-c3ccccc3)n2)cc1]

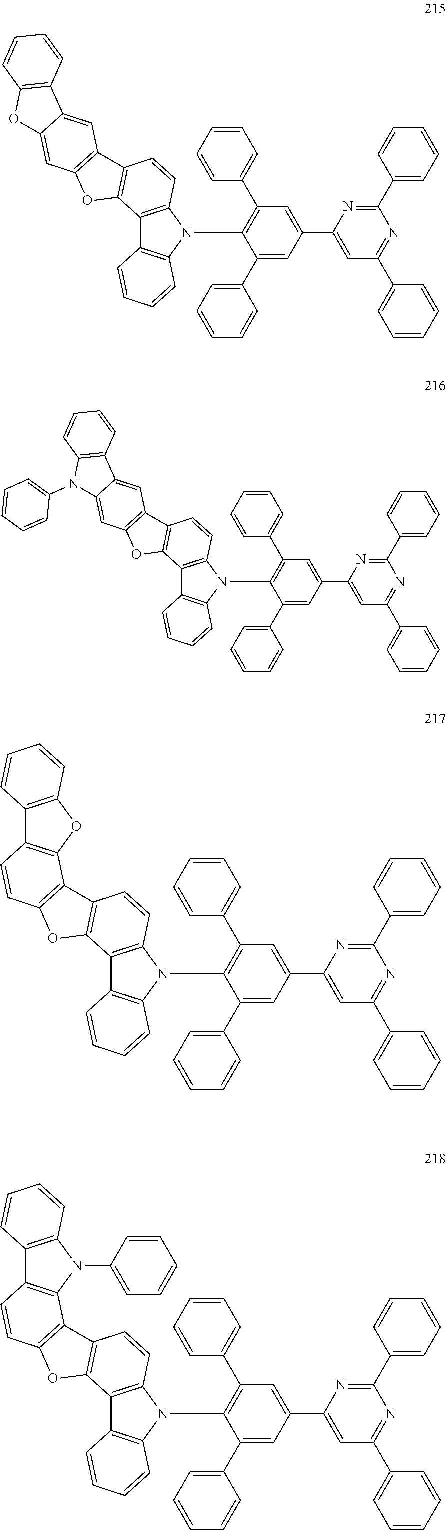 [SMILES: c1ccc(-c2cc(-c3cc(-c4ccccc4)c(-n4c5ccccc5c5c6oc7cc8oc9ccccc9c8cc7c6ccc54)c(-c4ccccc4)c3)nc(-c3ccccc3)n2)cc1 ; c1ccc(-c2cc(-c3cc(-c4ccccc4)c(-n4c5ccccc5c5c6oc7cc8c(cc7c6ccc54)c4ccccc4n8-c4ccccc4)c(-c4ccccc4)c3)nc(-c3ccccc3)n2)cc1 ; c1ccc(-c2cc(-c3cc(-c4ccccc4)c(-n4c5ccccc5c5c6oc7ccc8c9ccccc9oc8c7c6ccc54)c(-c4ccccc4)c3)nc(-c3ccccc3)n2)cc1 ; c1ccc(-c2cc(-c3cc(-c4ccccc4)c(-n4c5ccccc5c5c6oc7ccc8c9ccccc9n(-c9ccccc9)c8c7c6ccc54)c(-c4ccccc4)c3)nc(-c3ccccc3)n2)cc1]

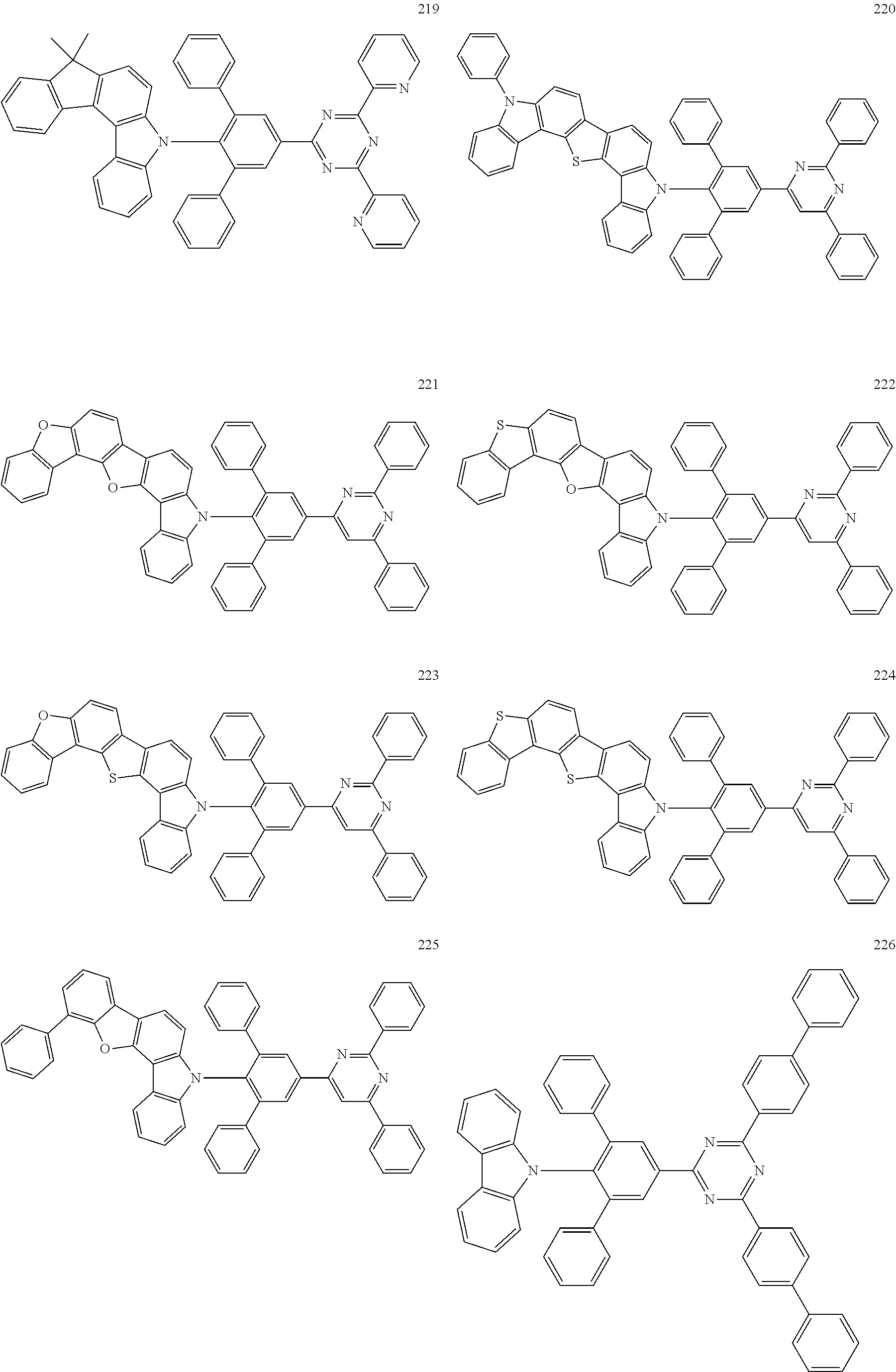

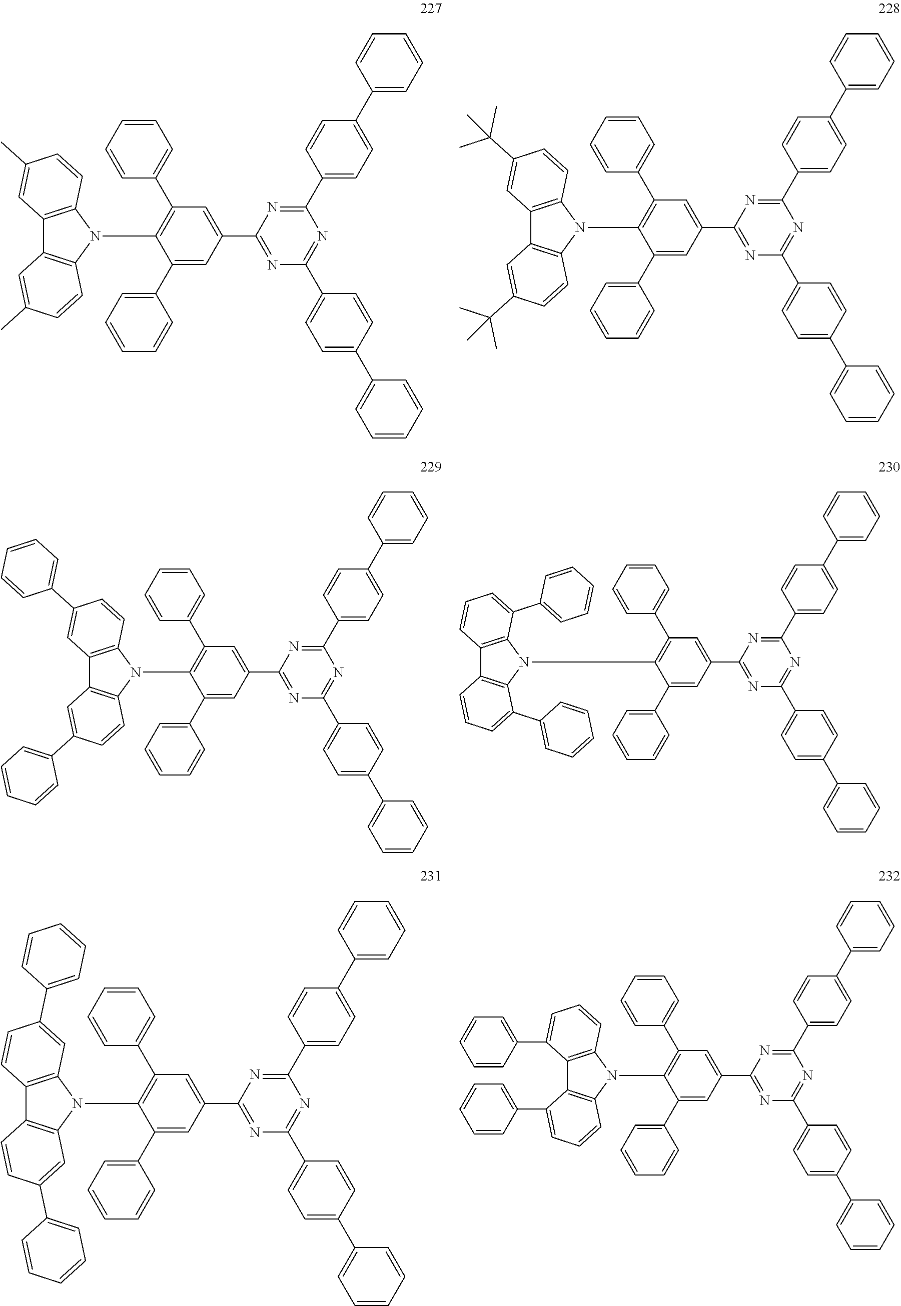

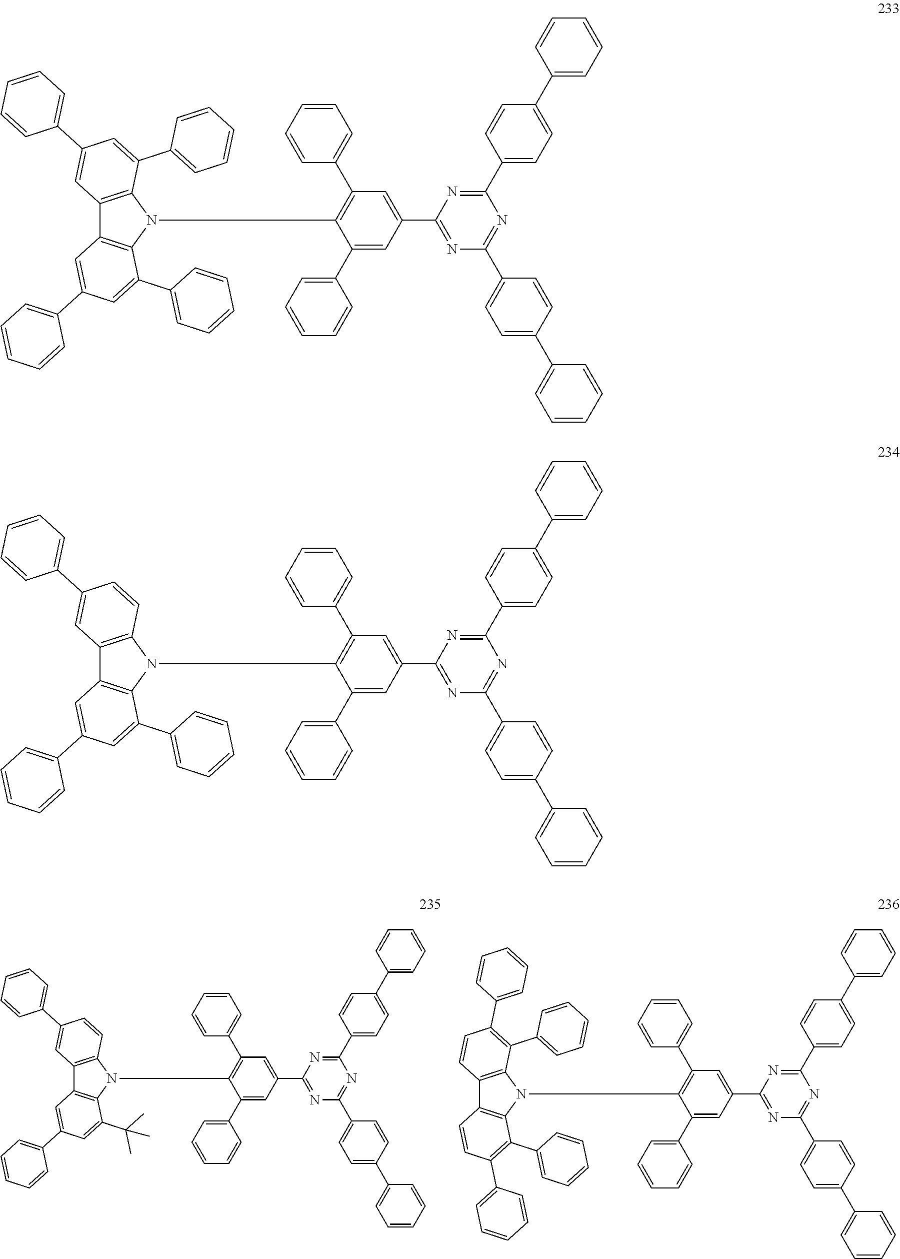

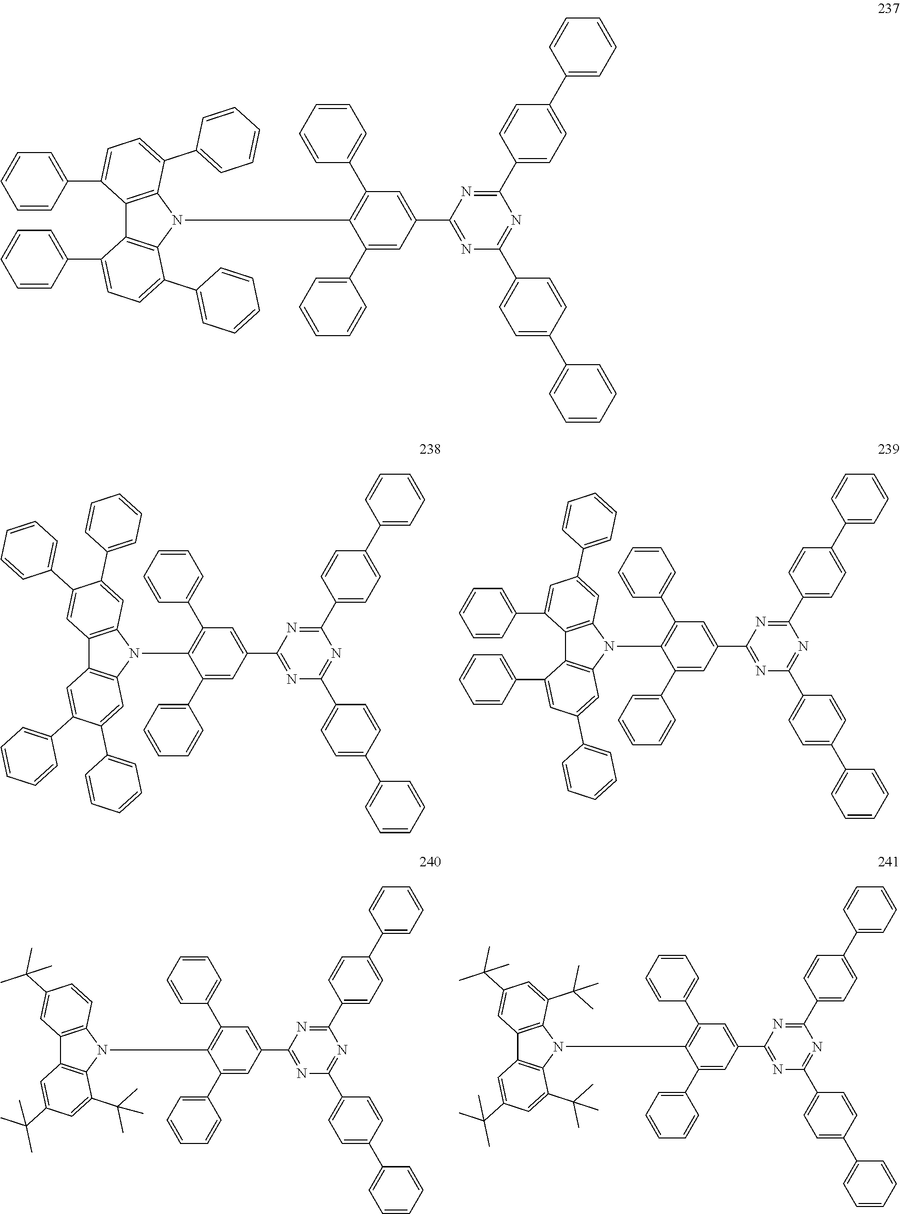

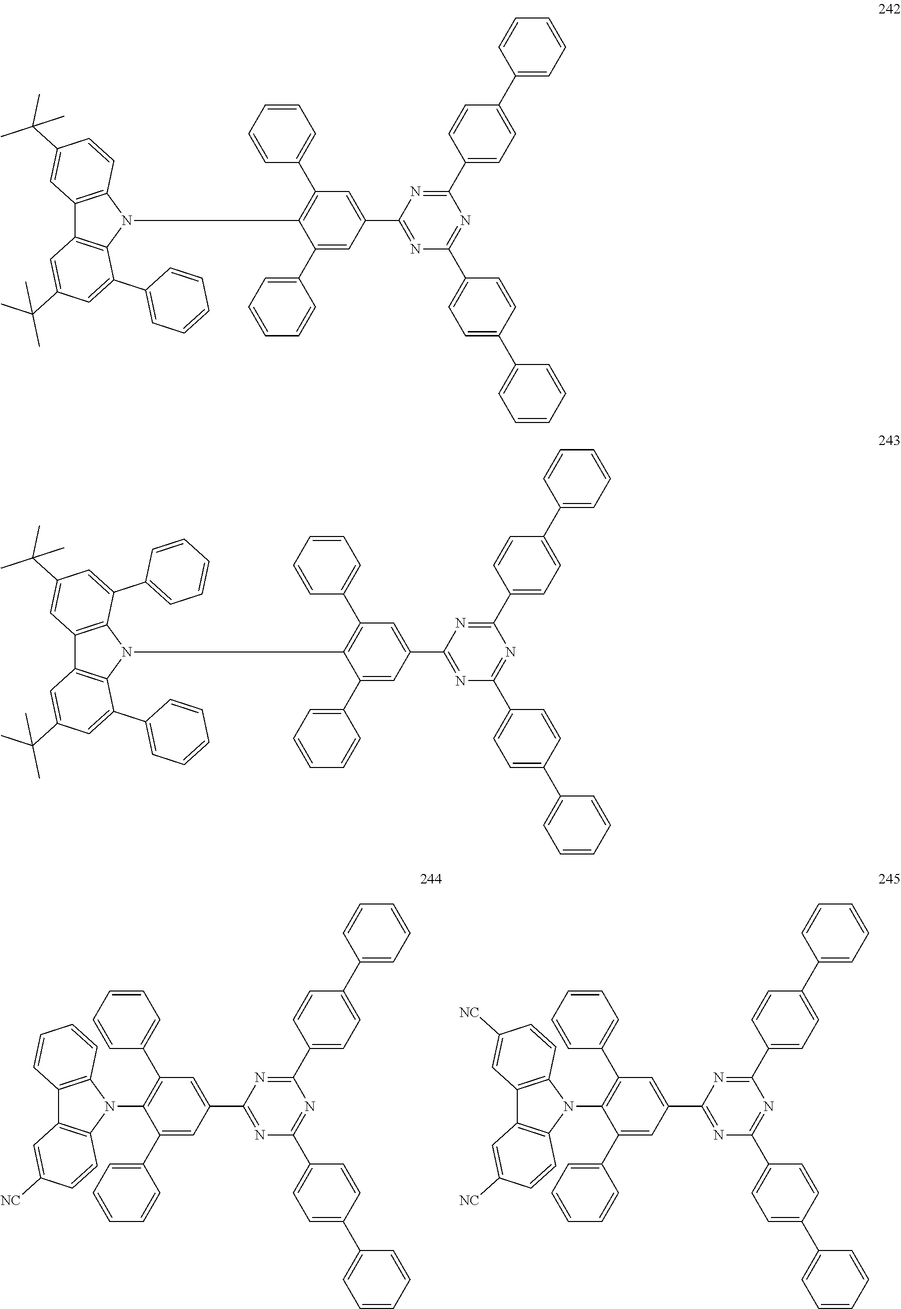

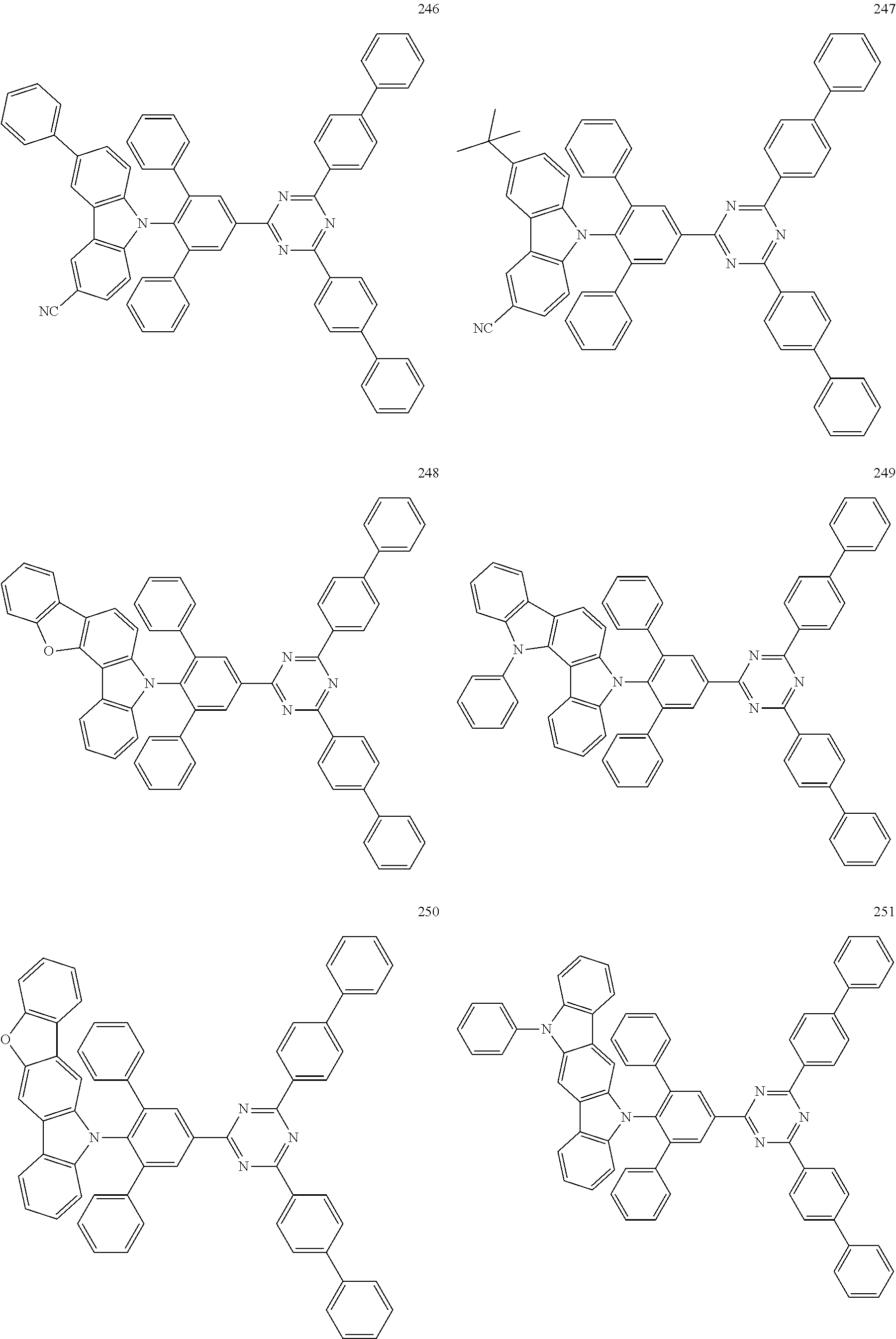

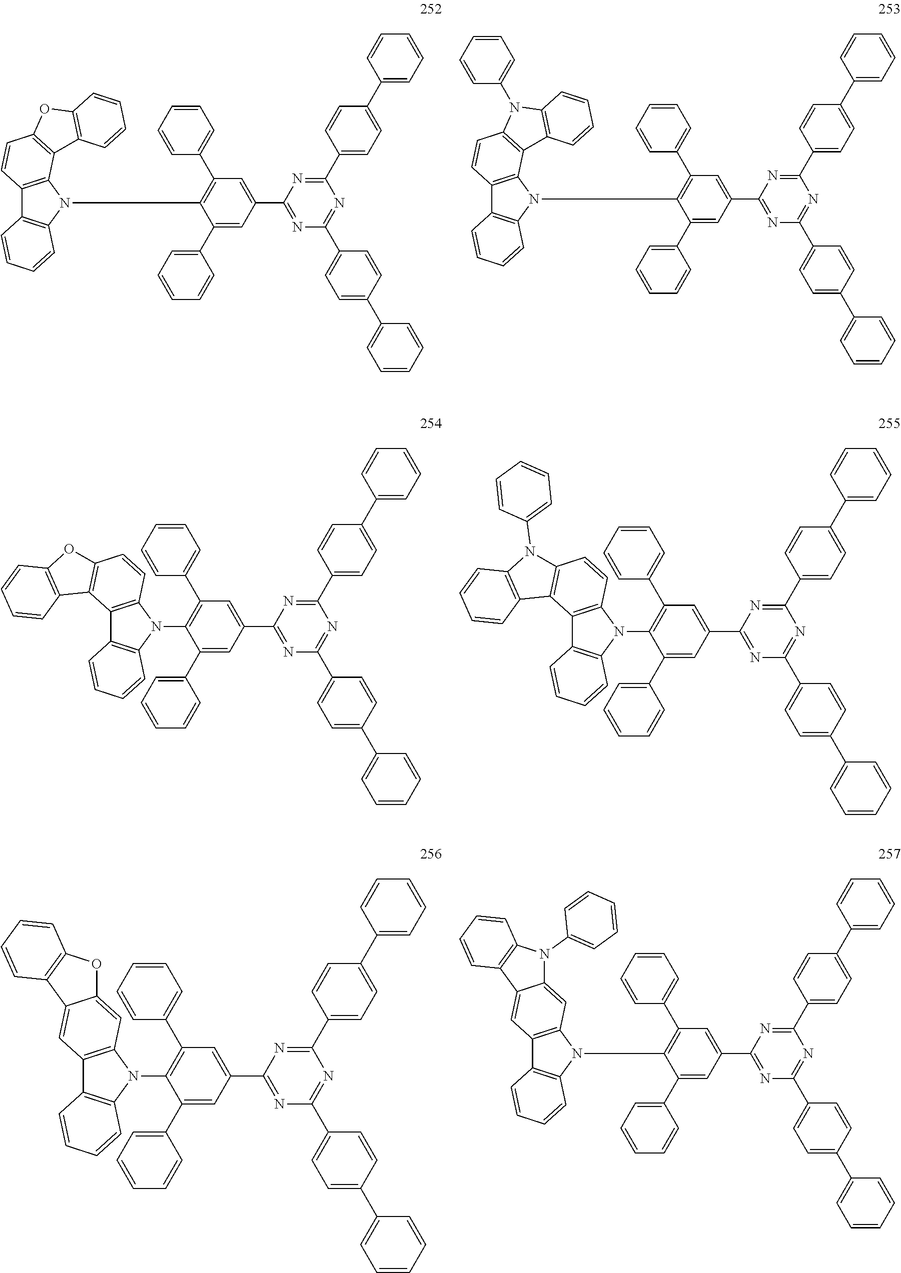

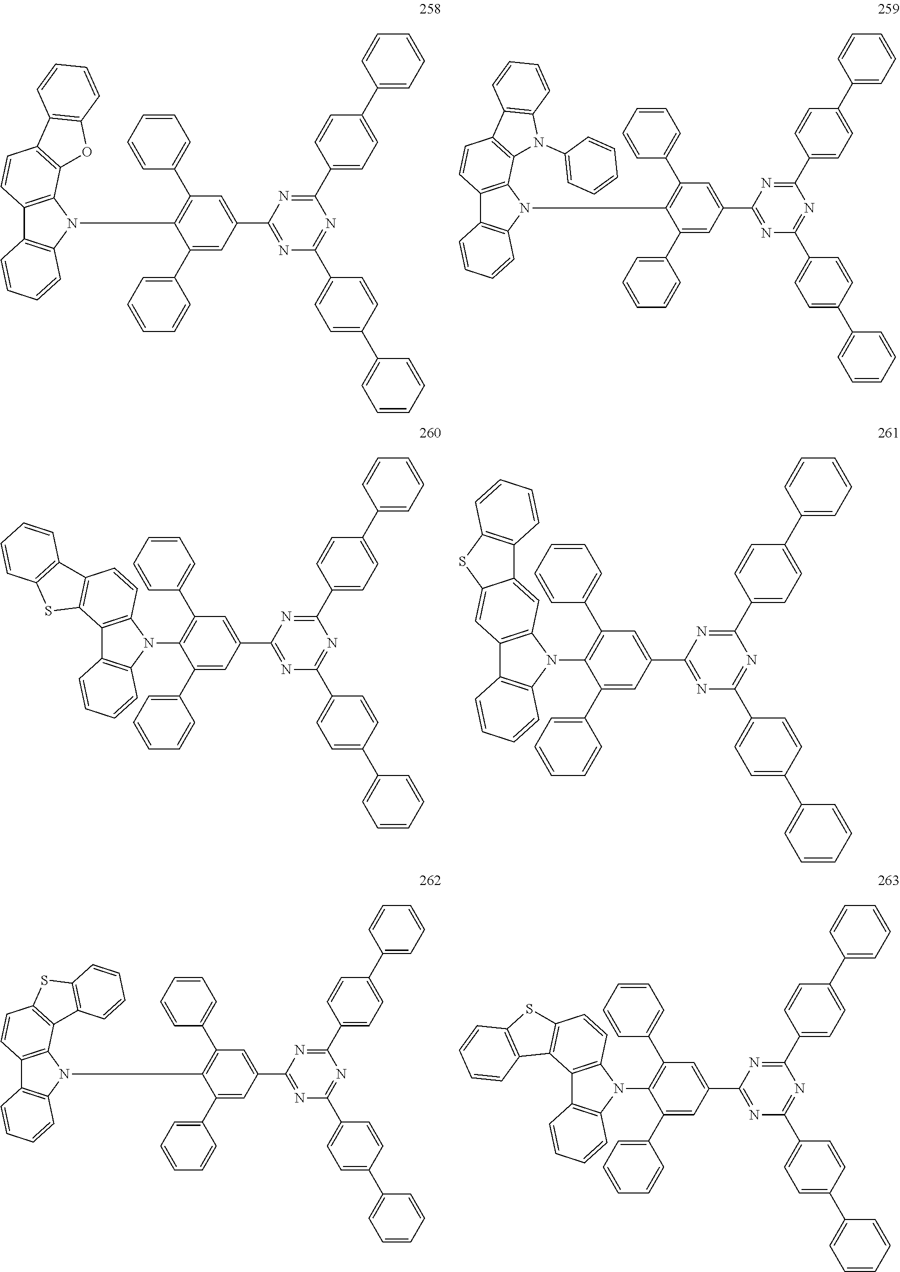

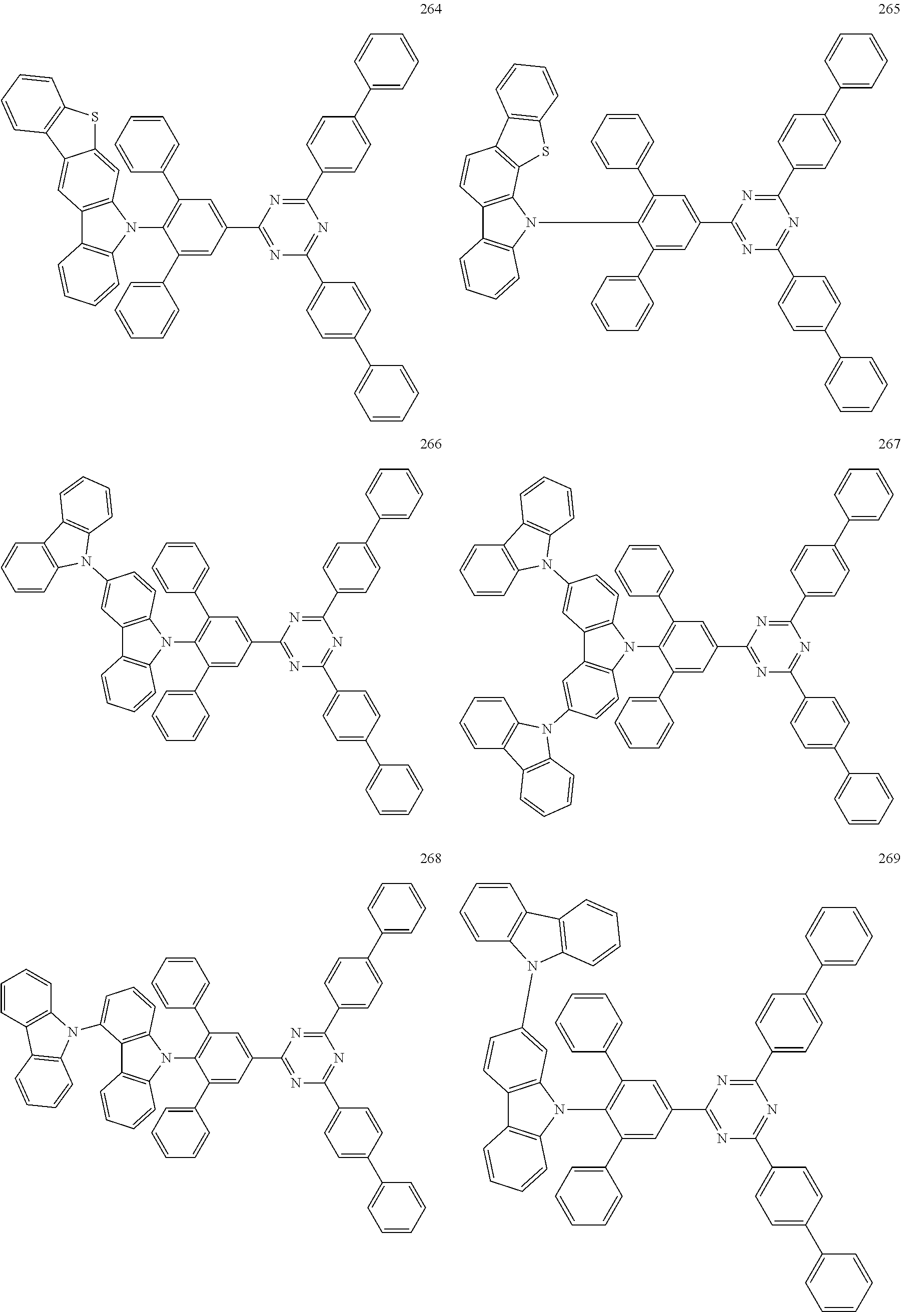

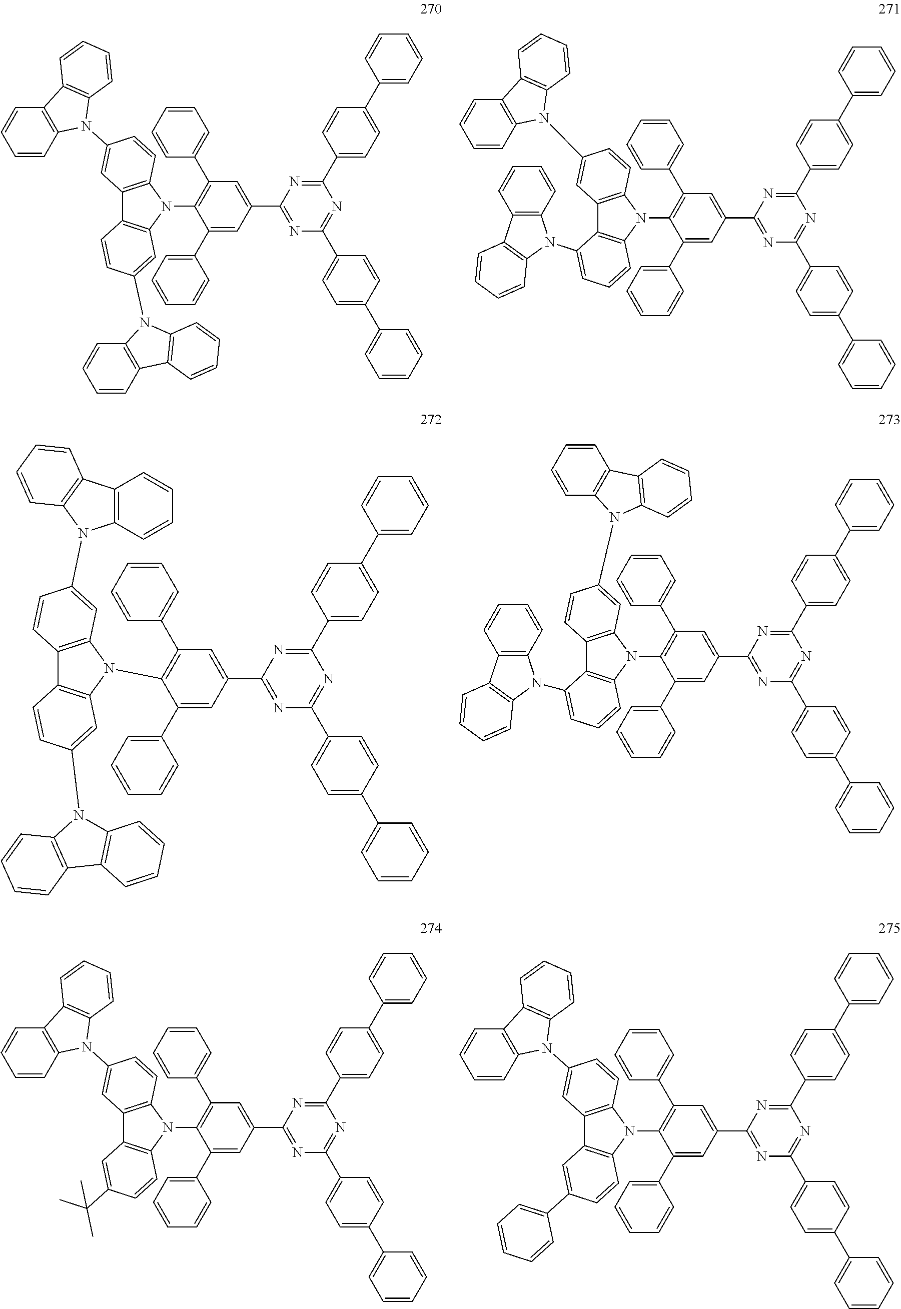

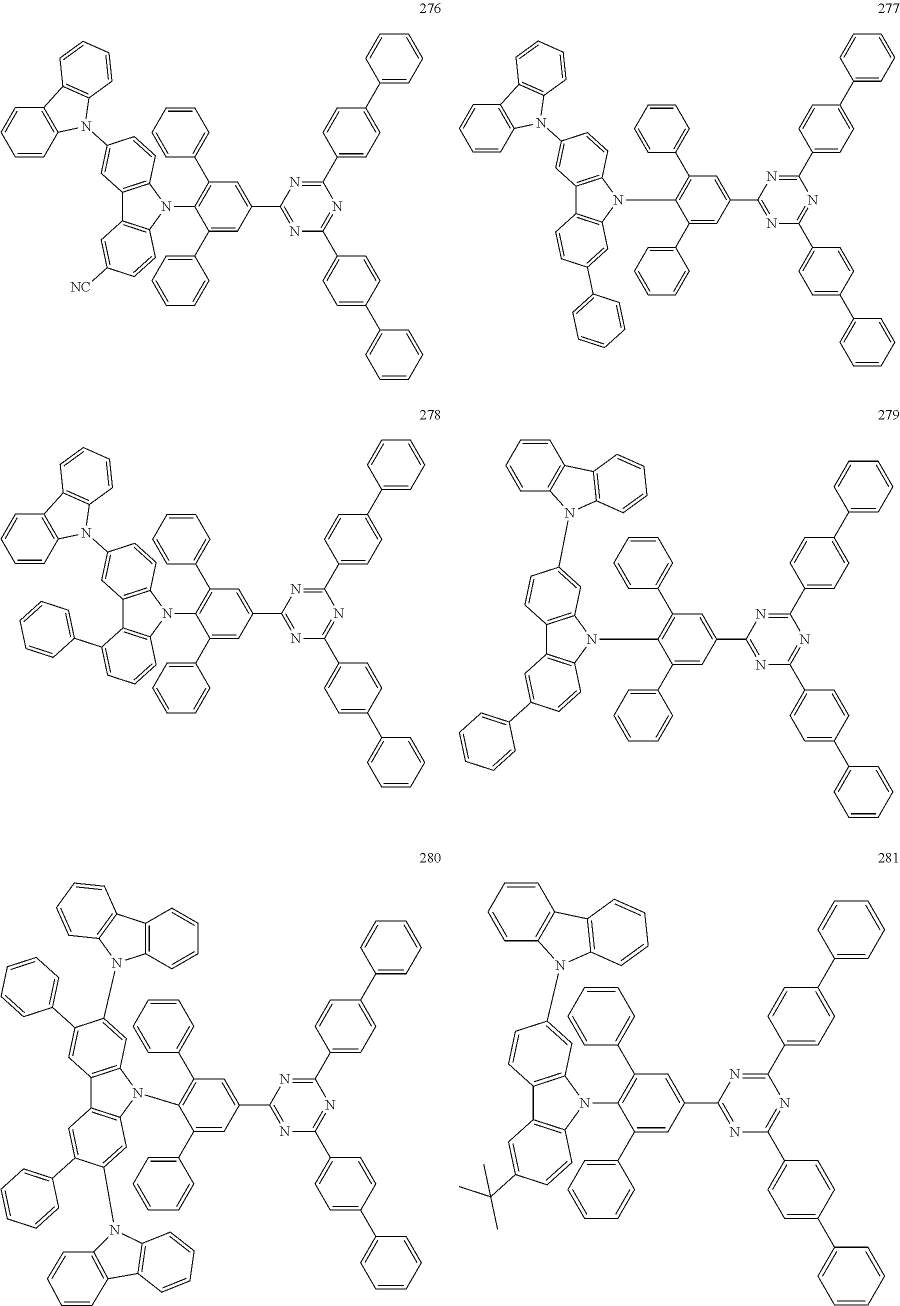

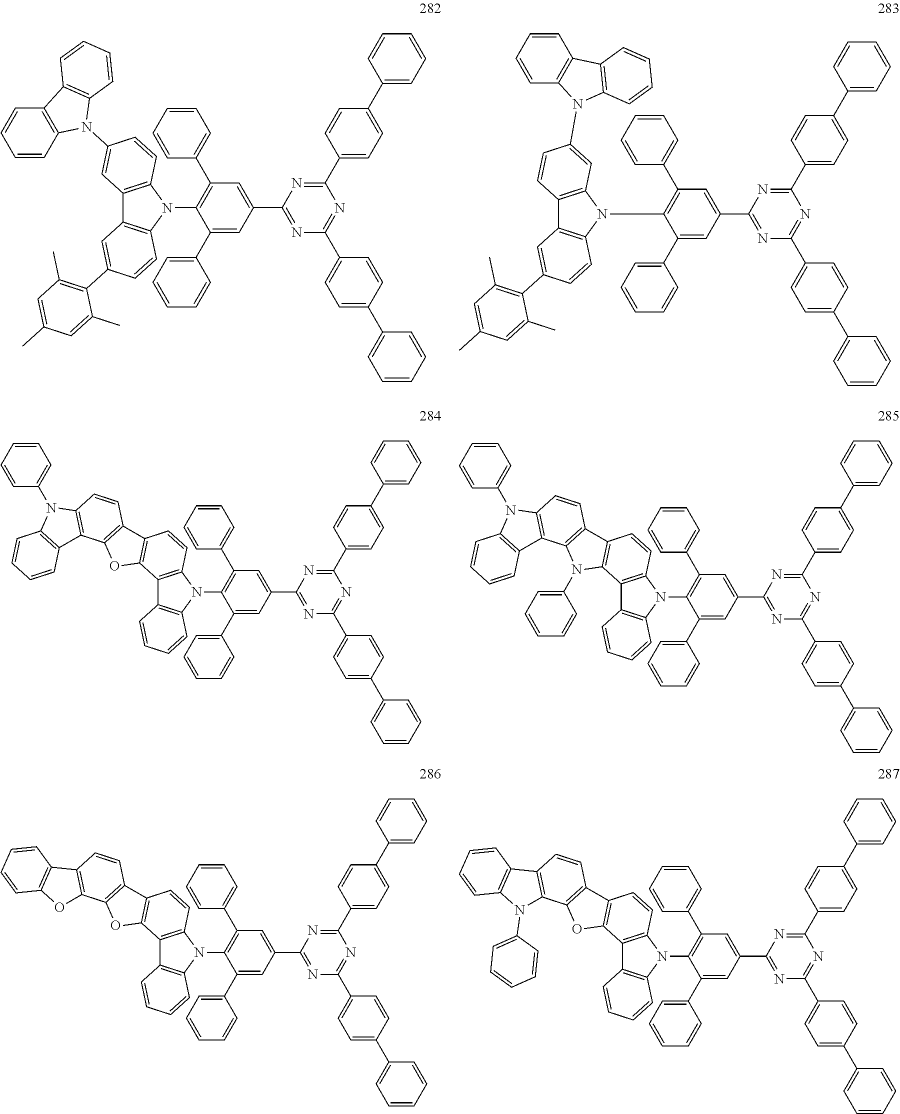

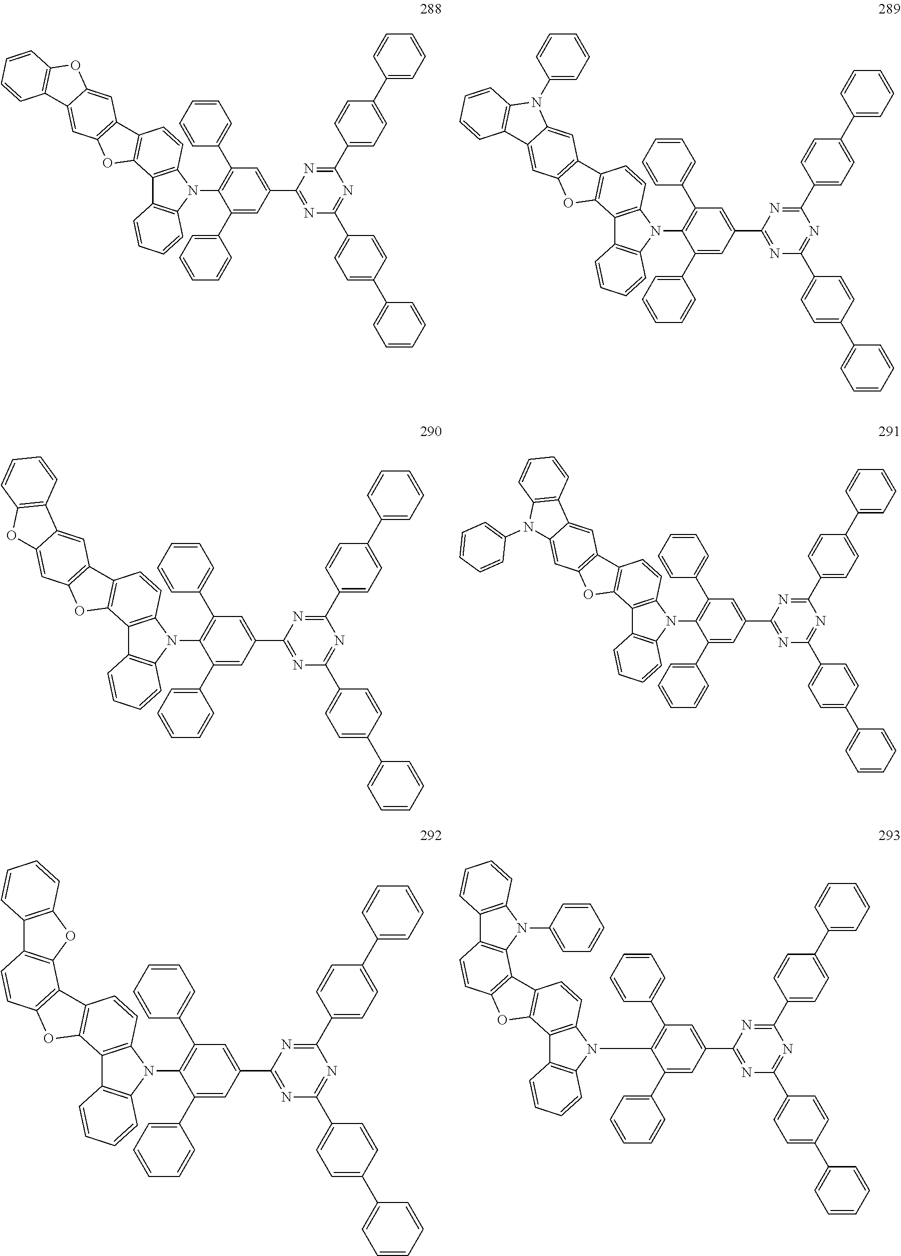

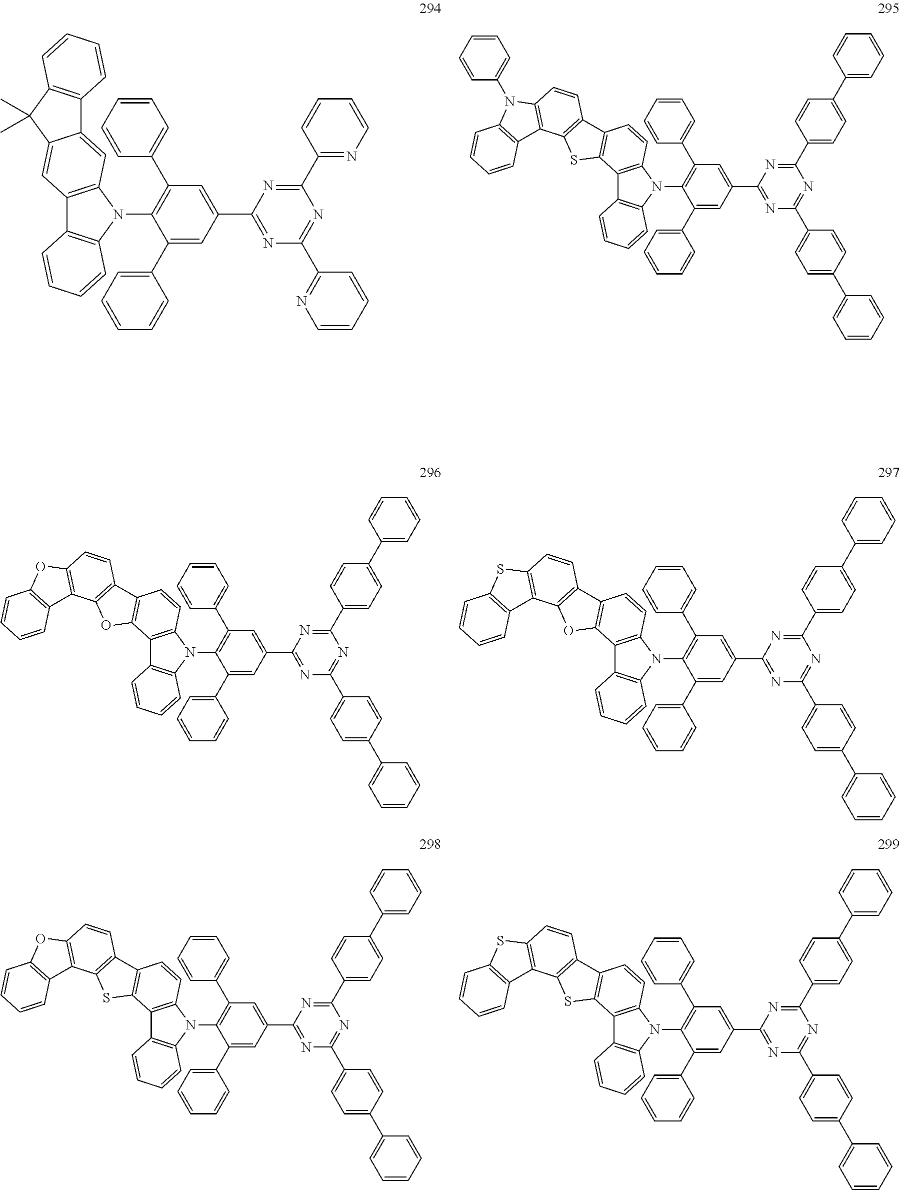

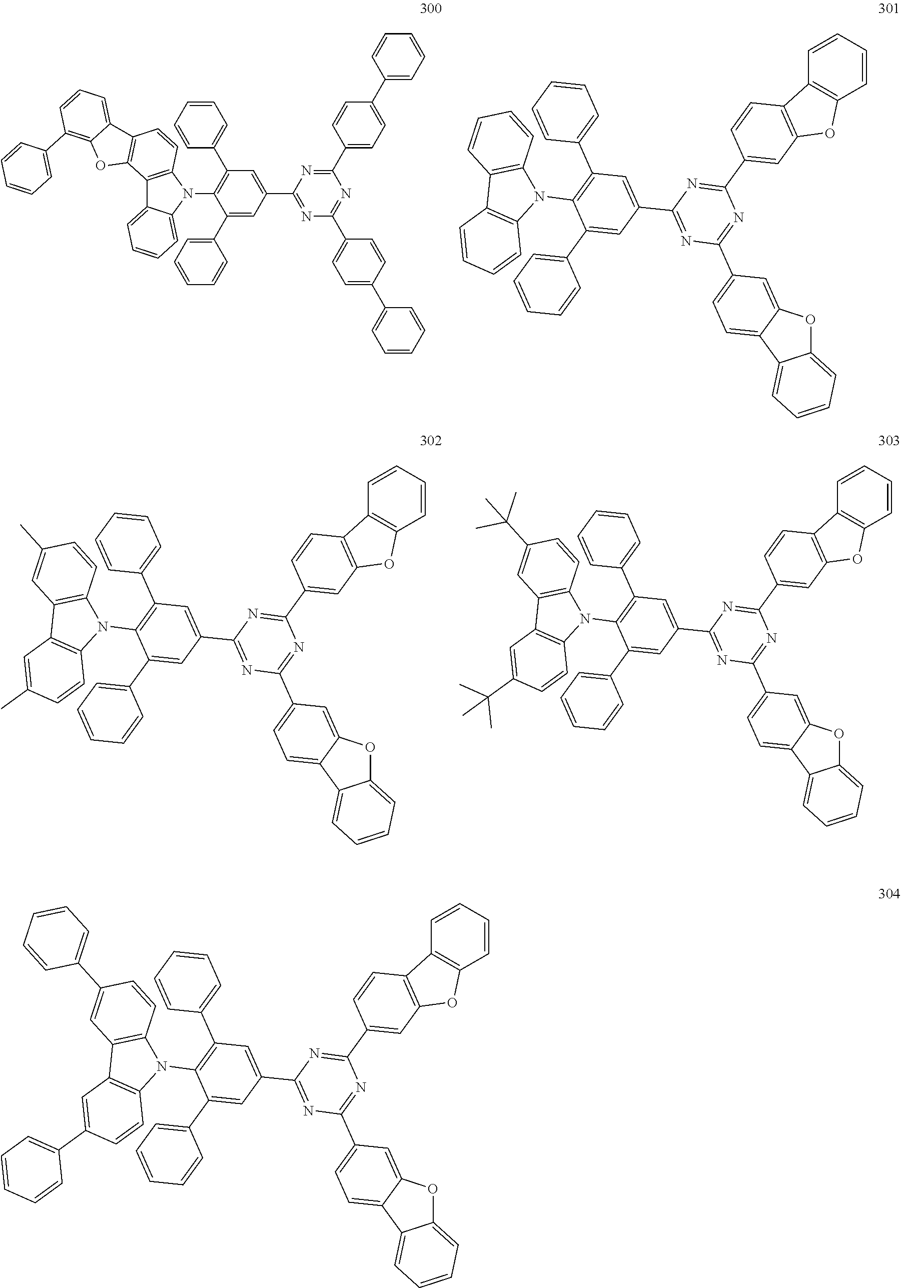

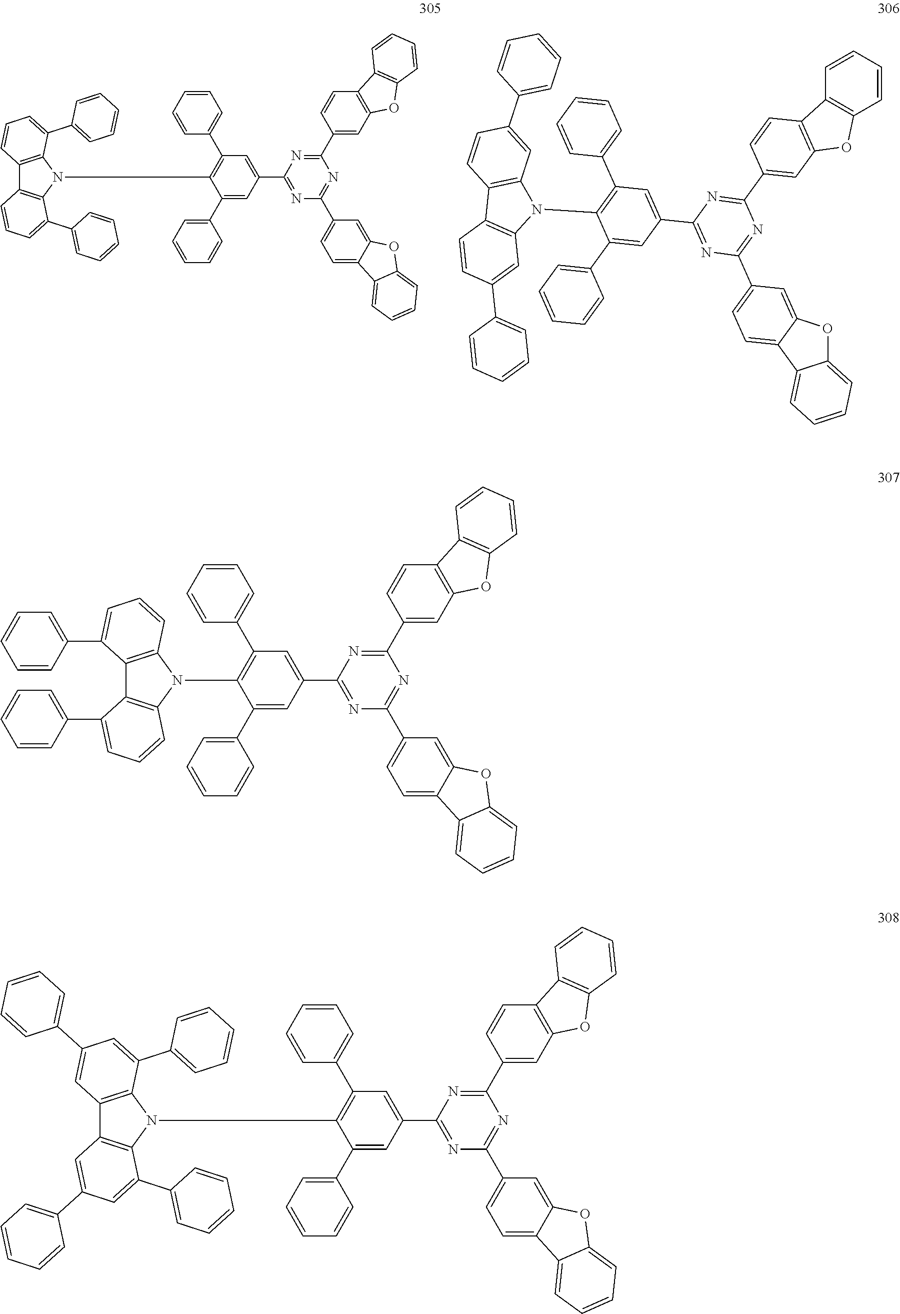

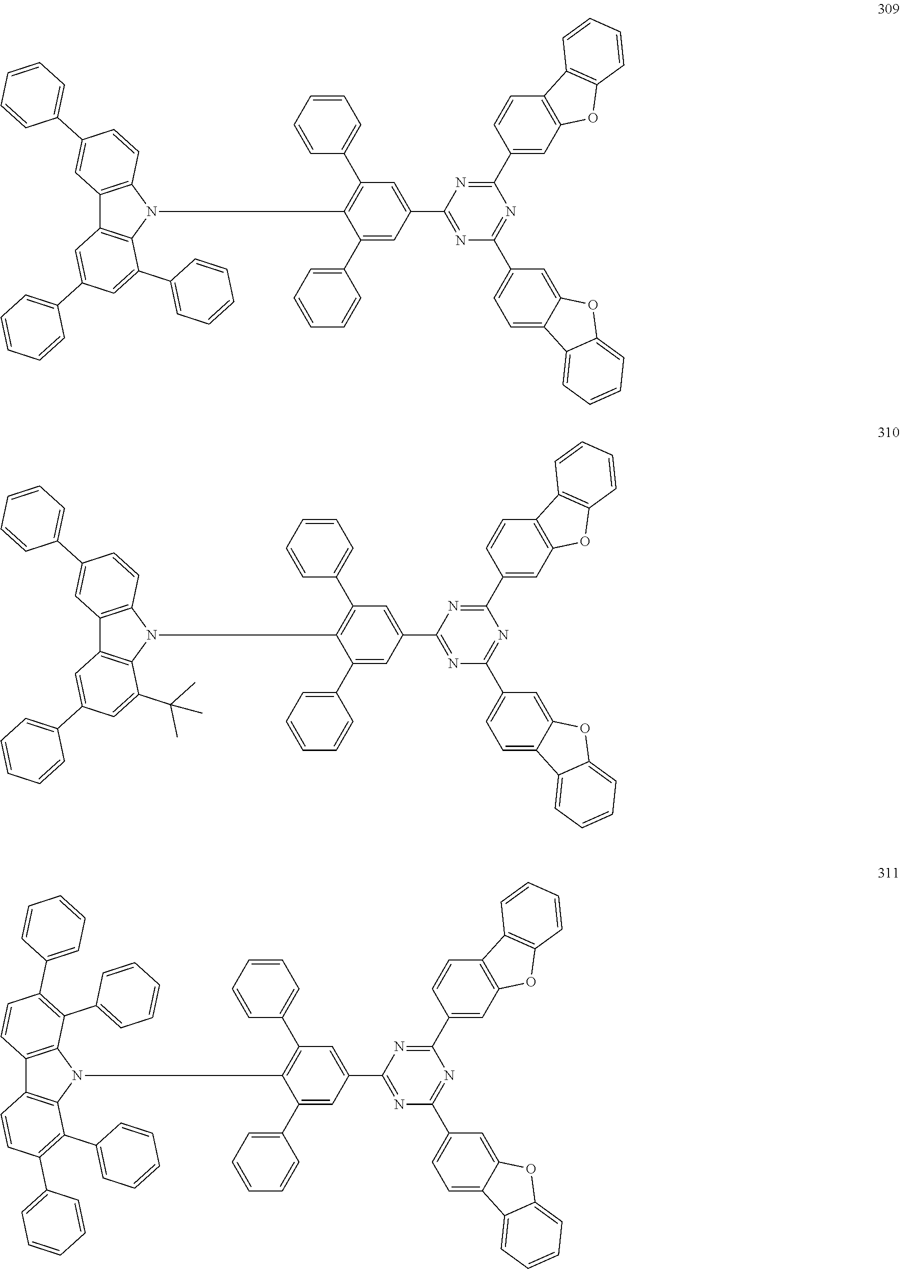

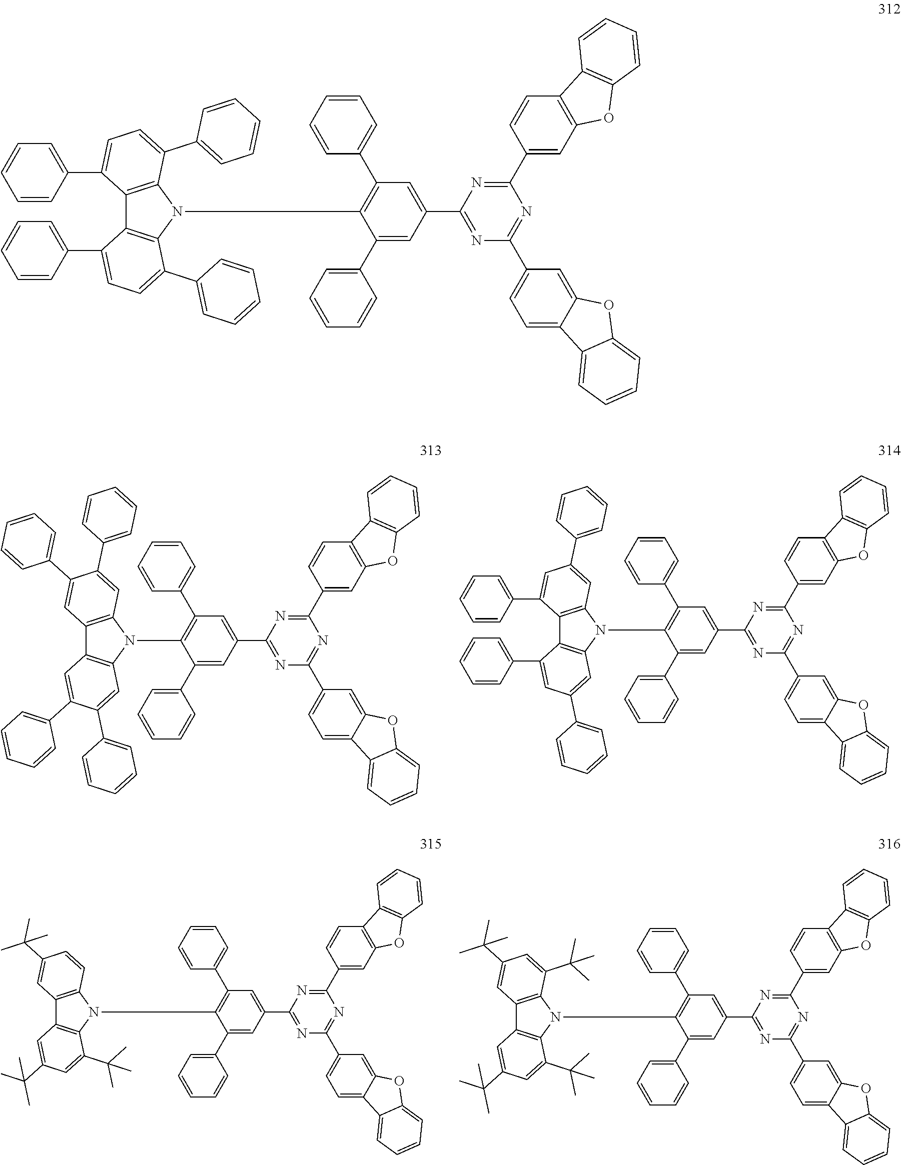

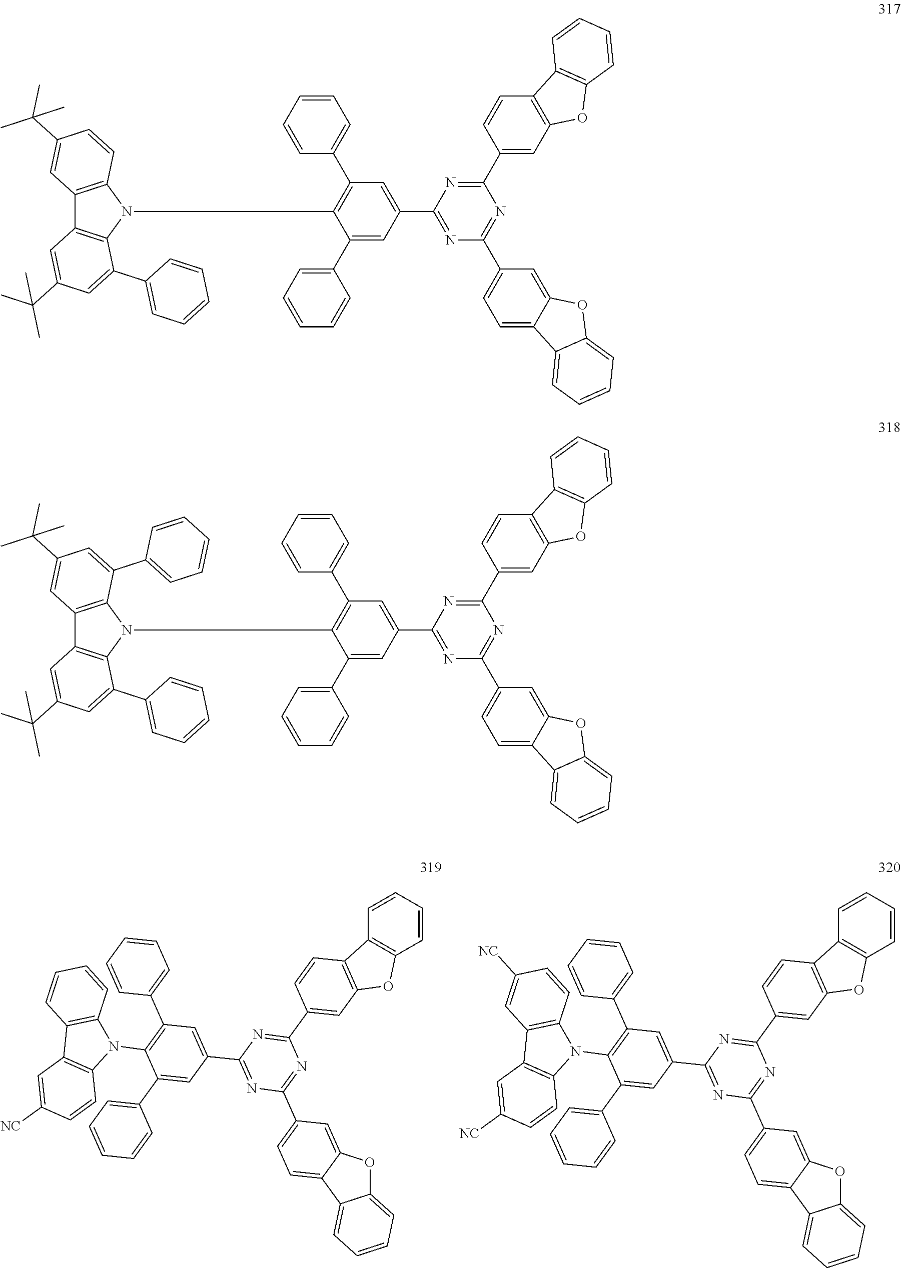

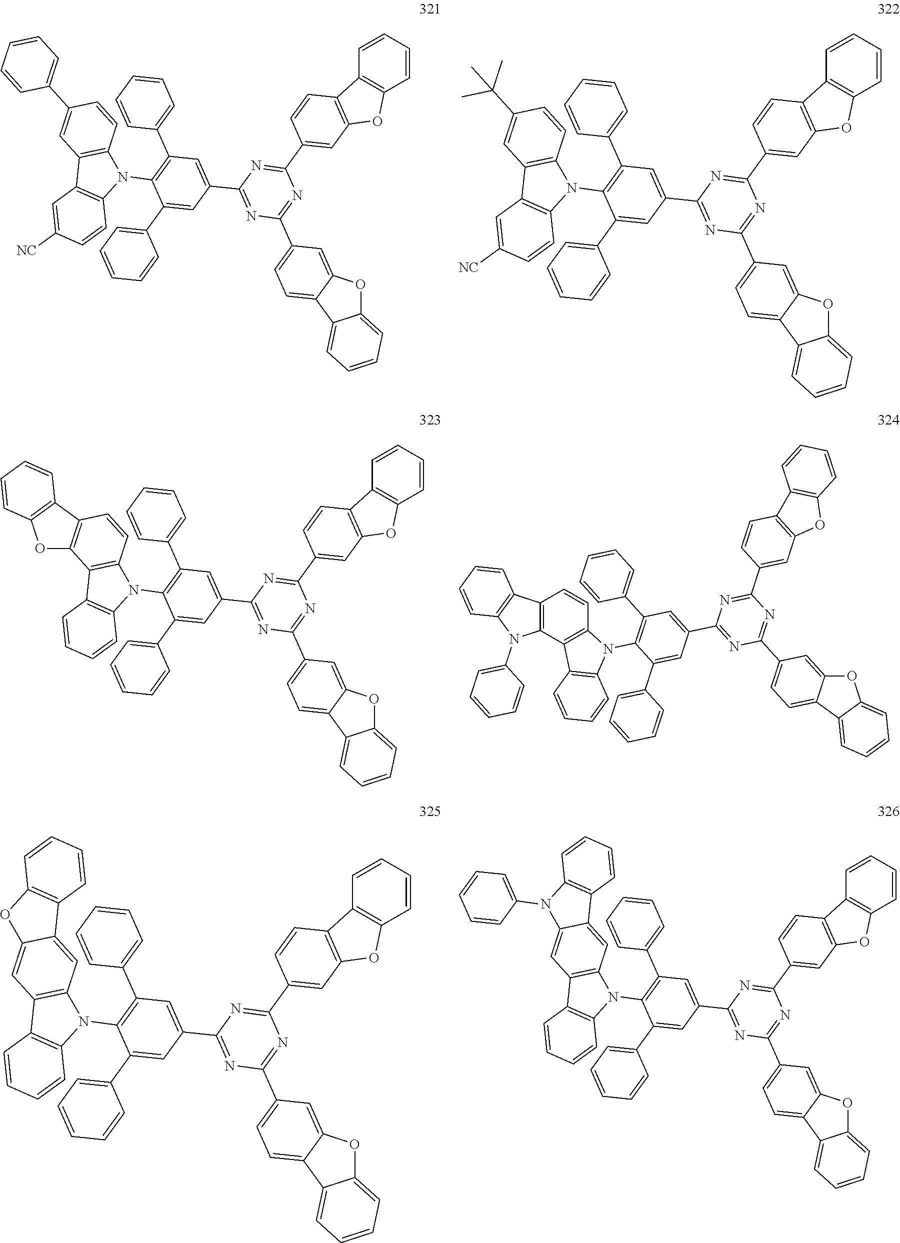

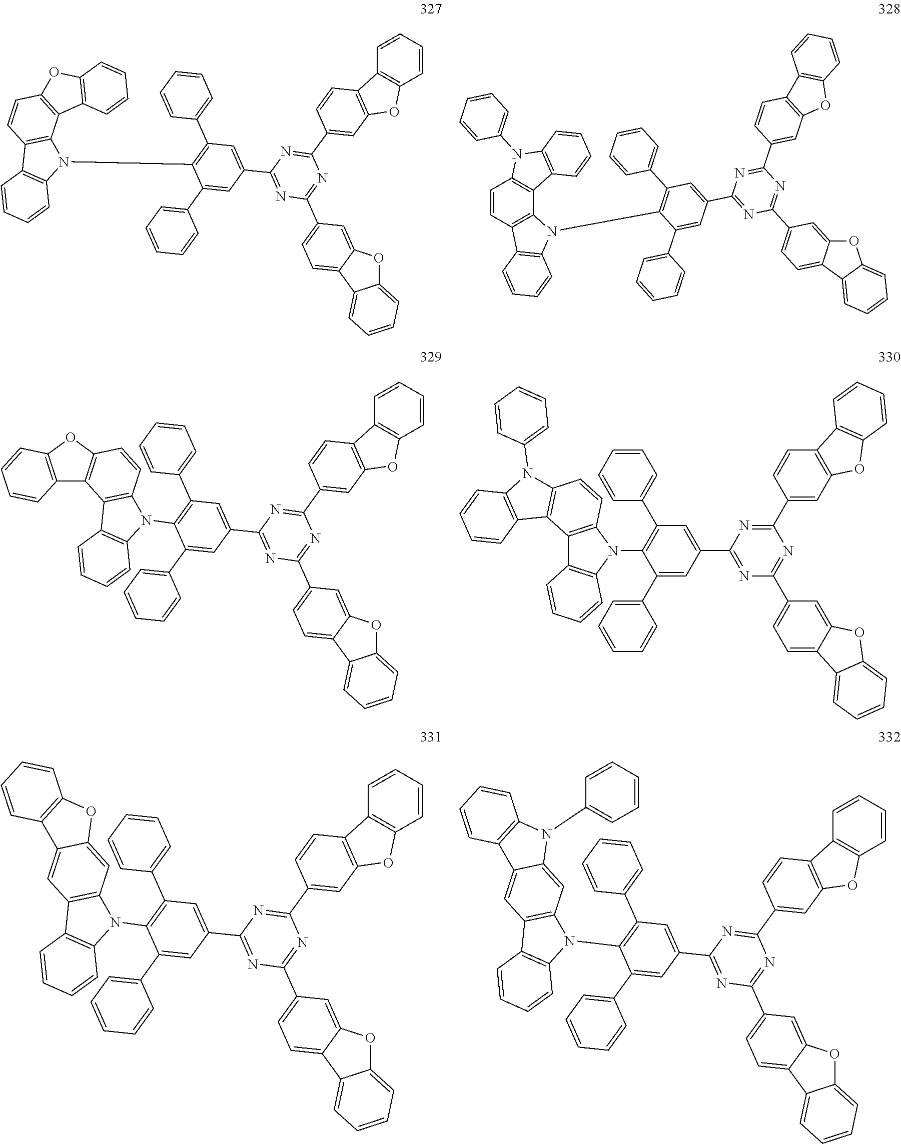

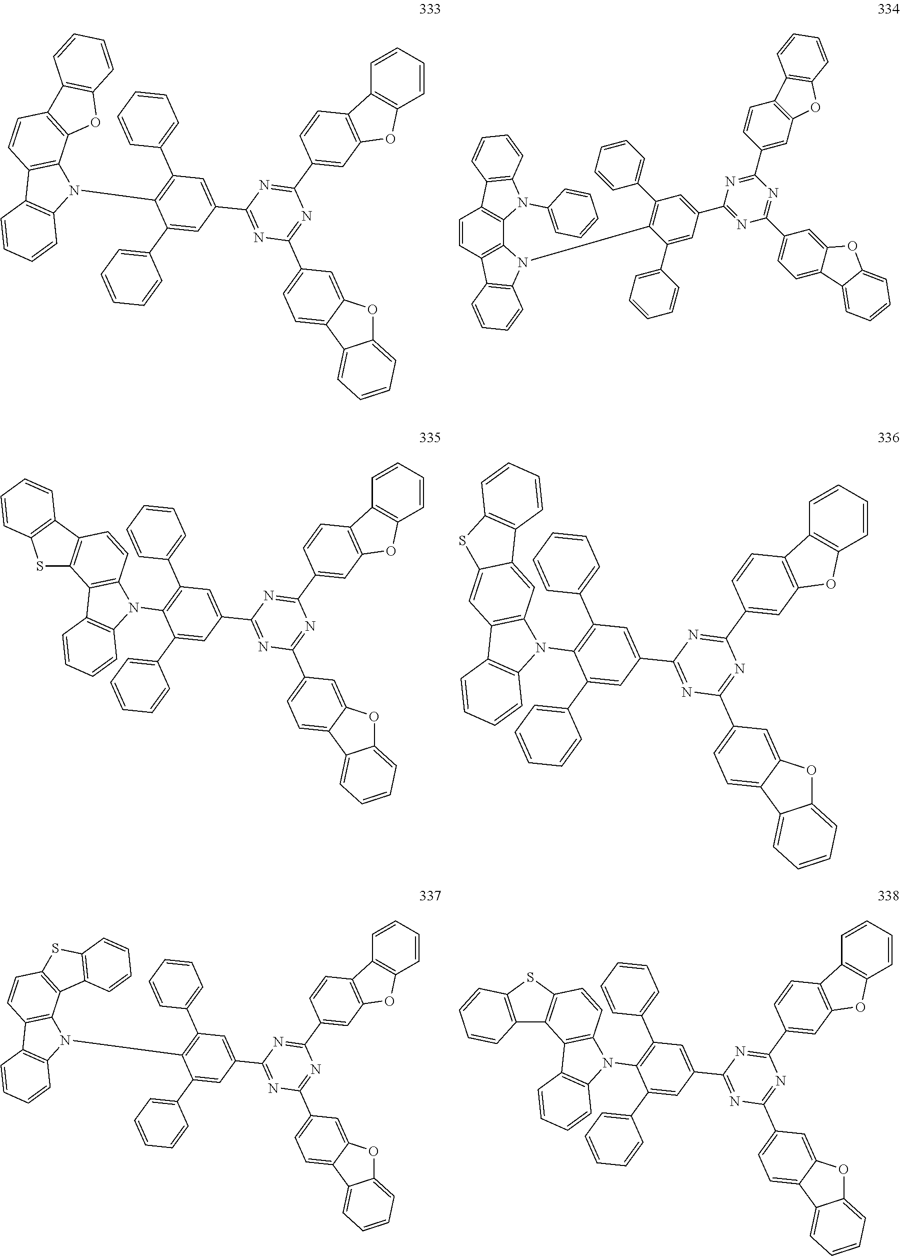

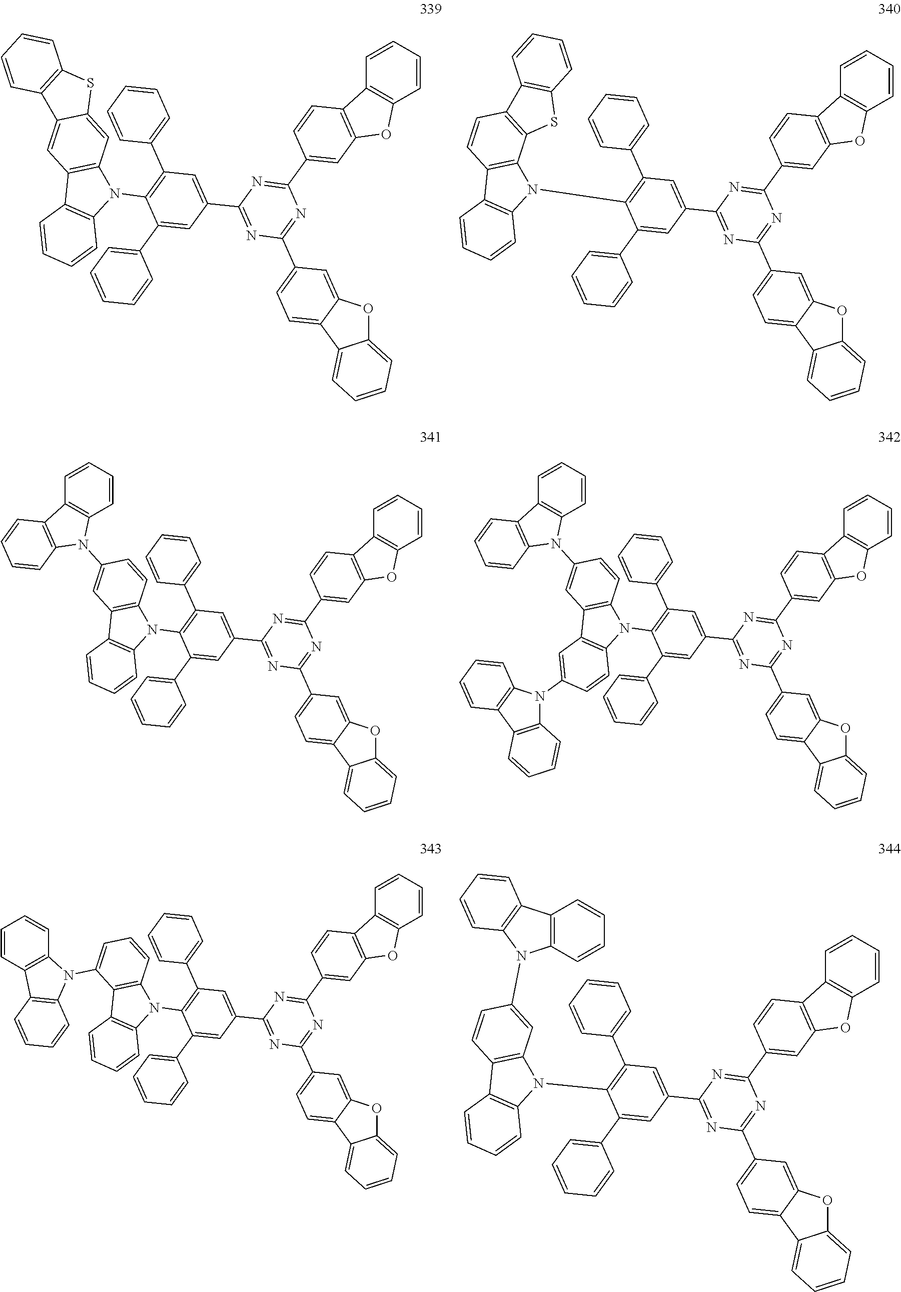

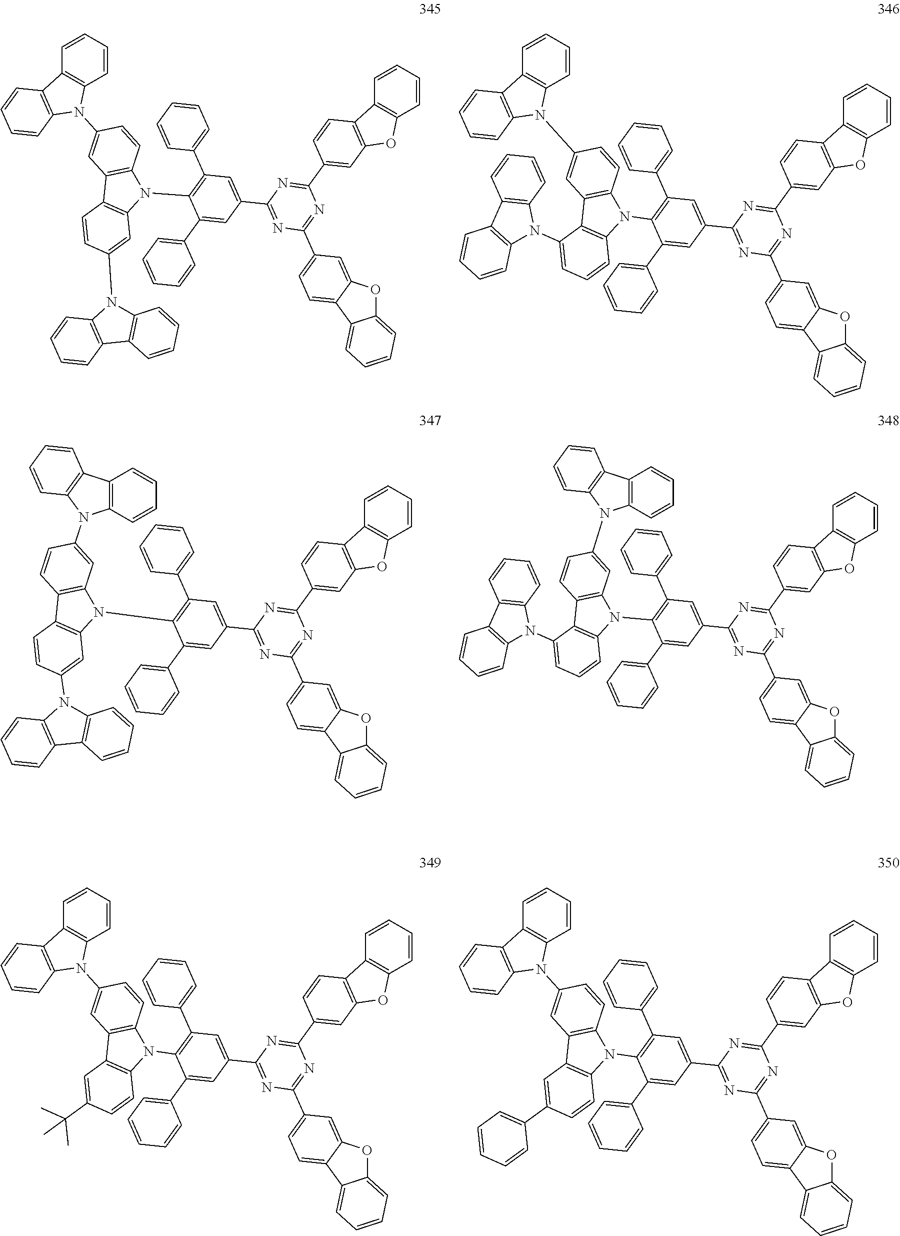

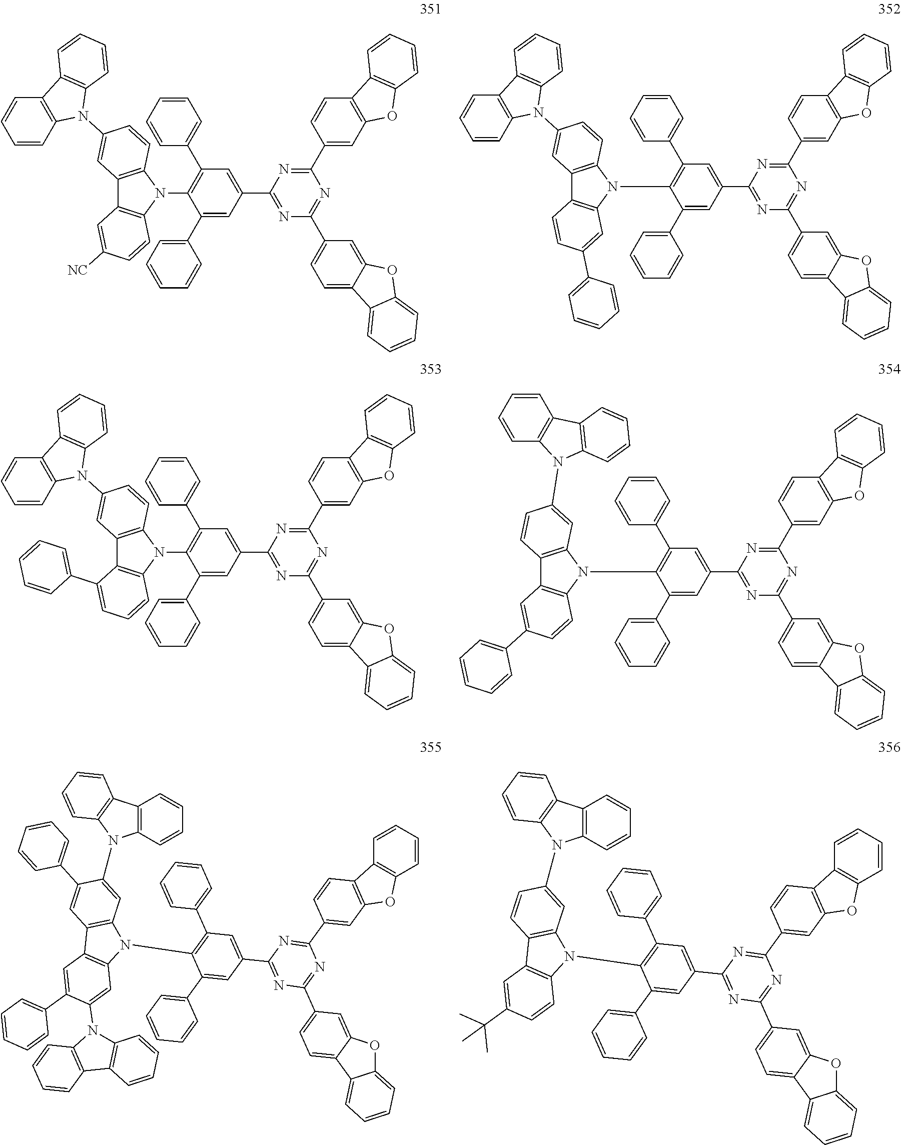

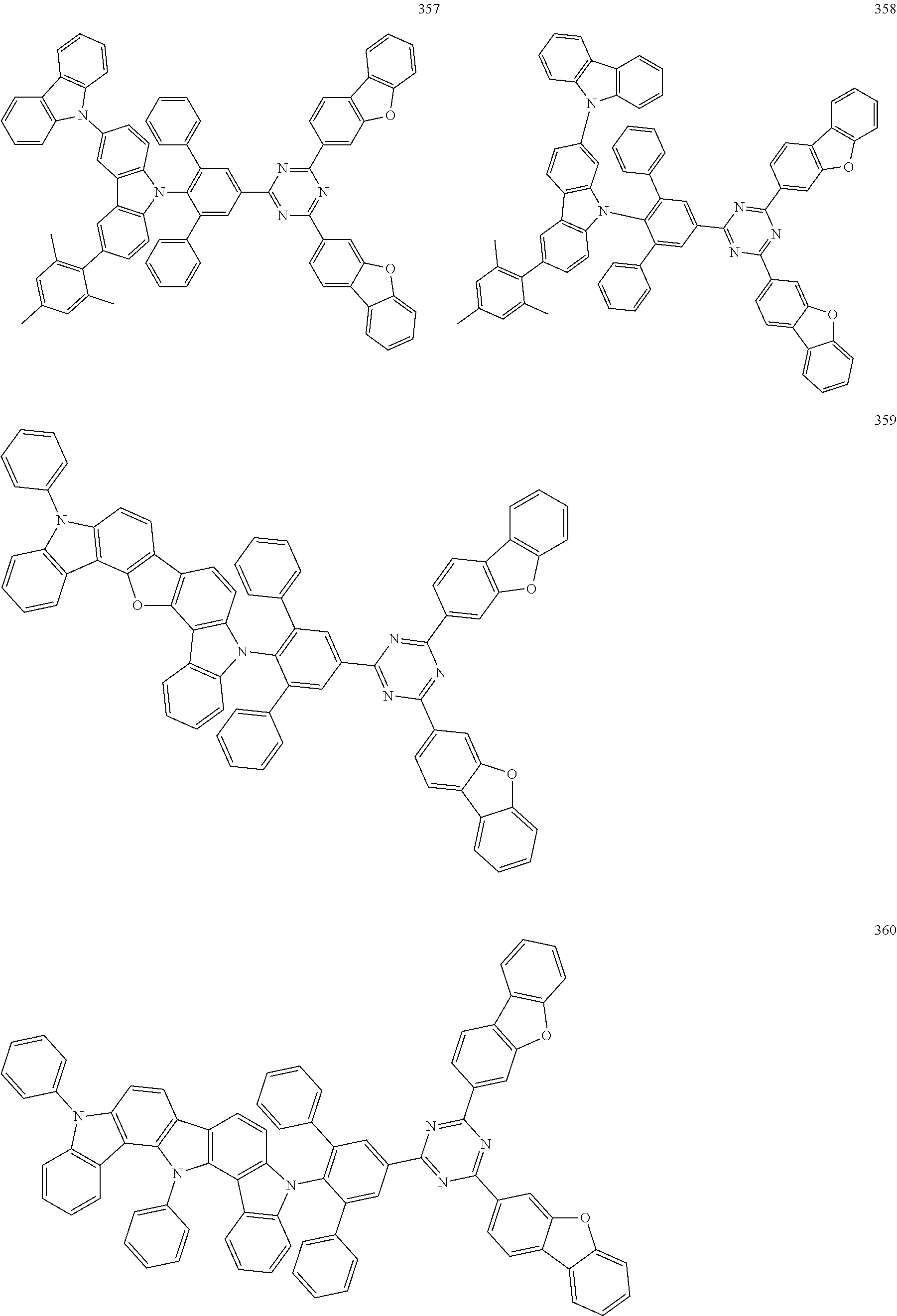

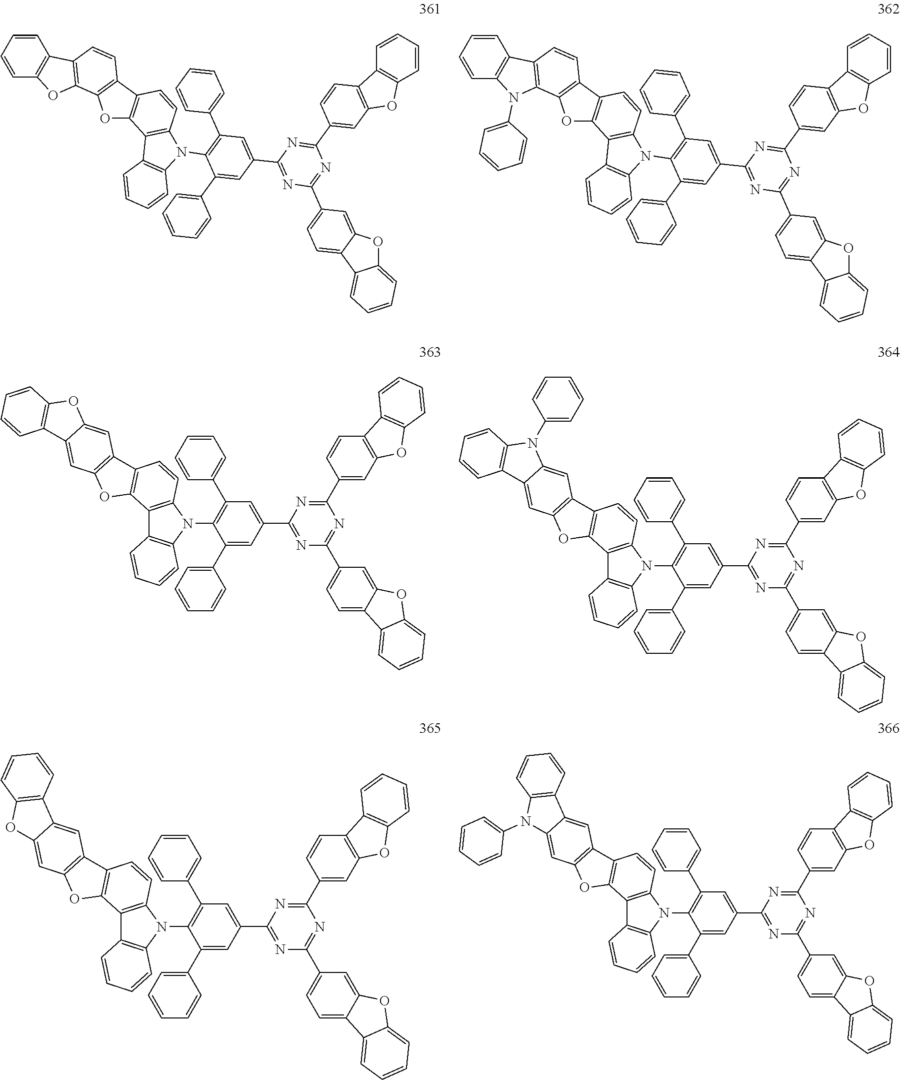

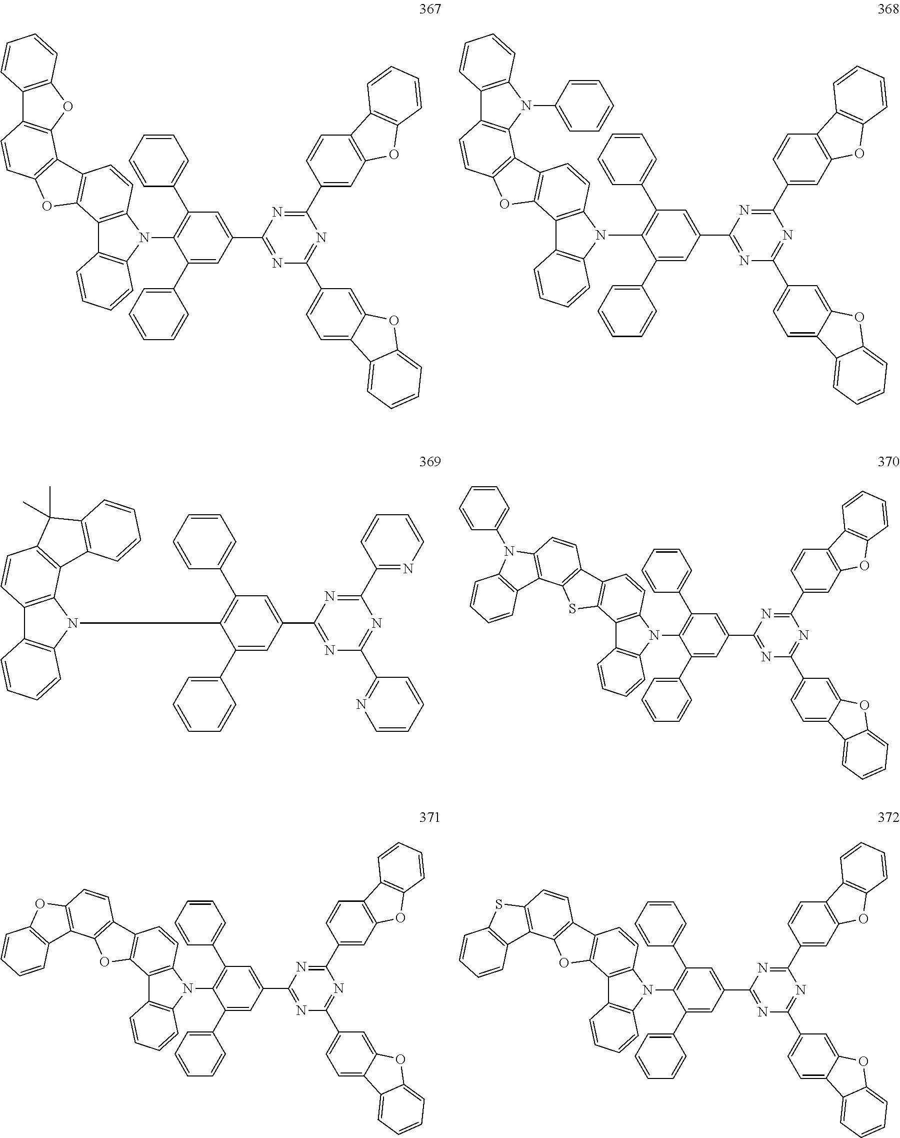

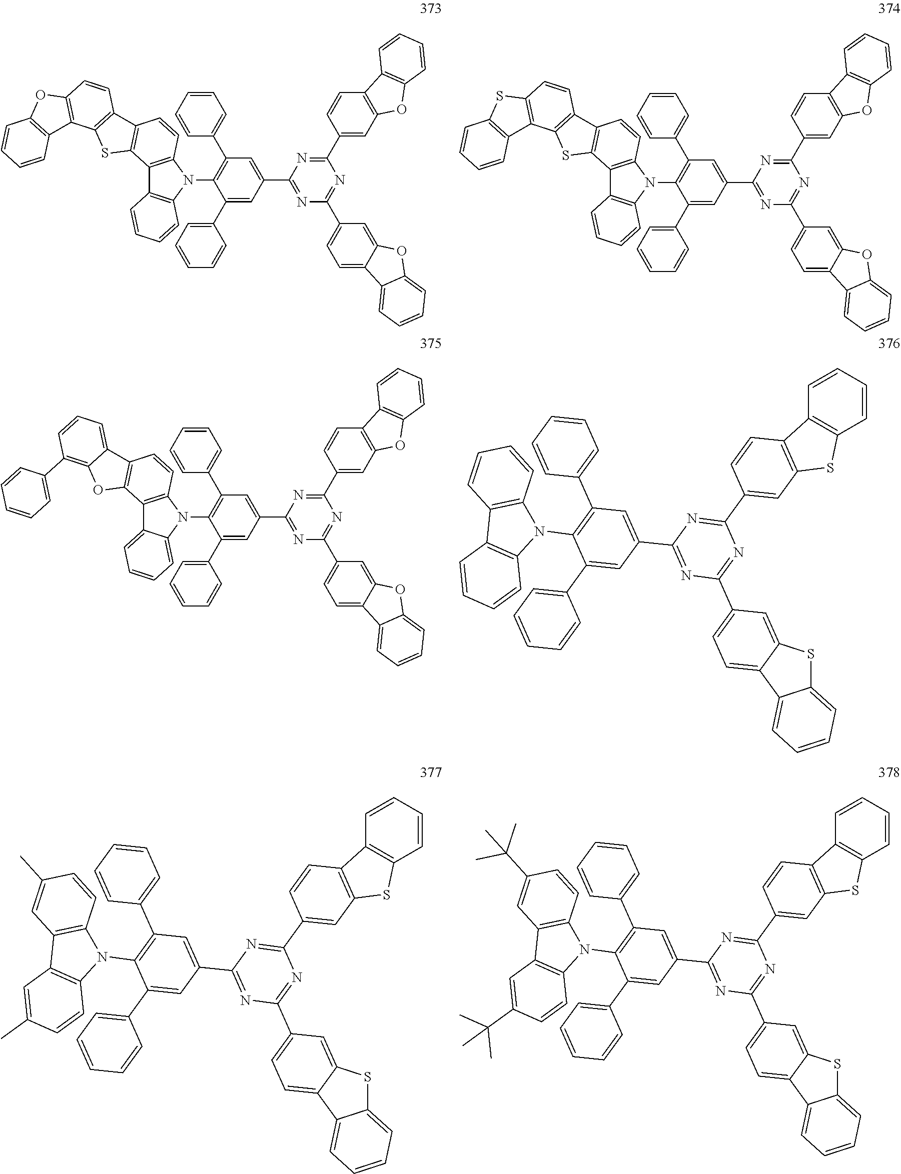

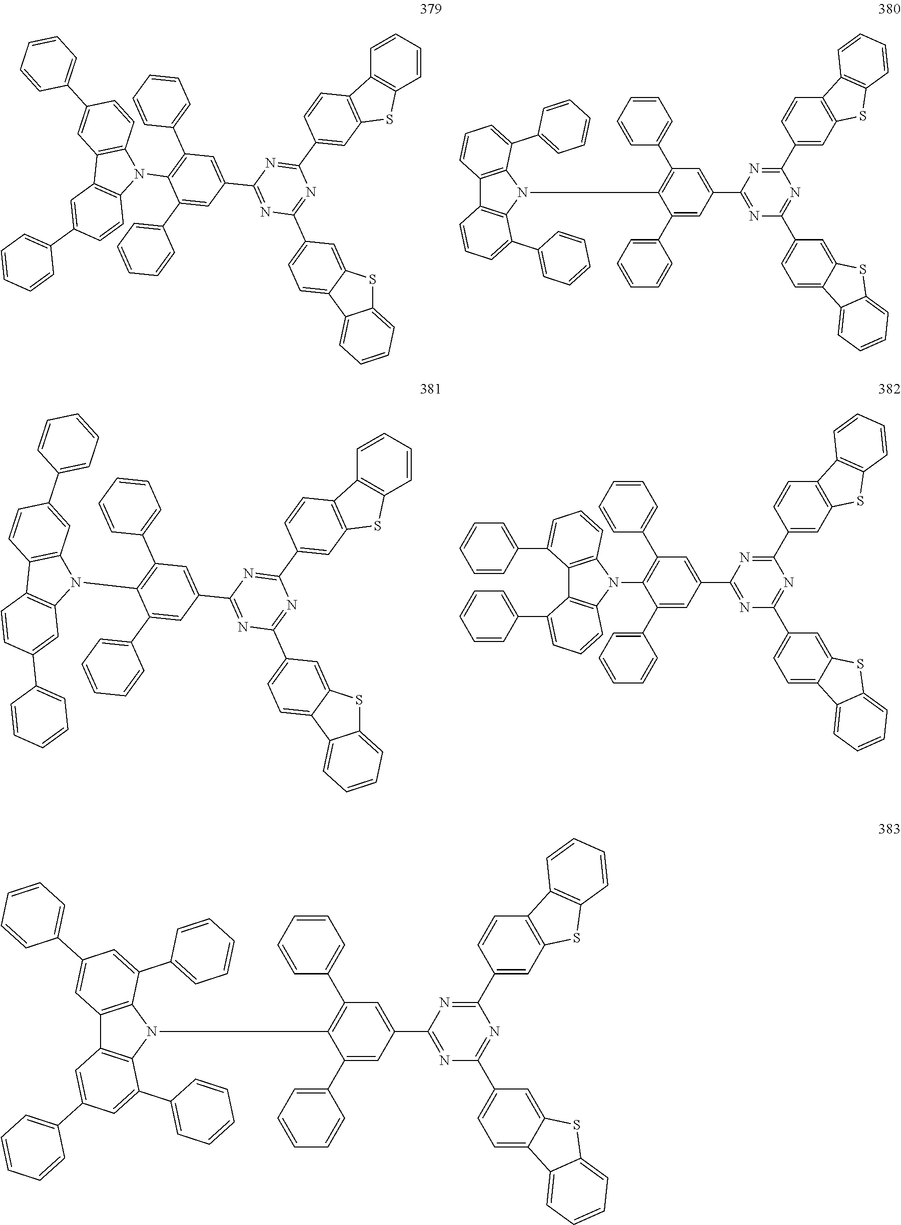

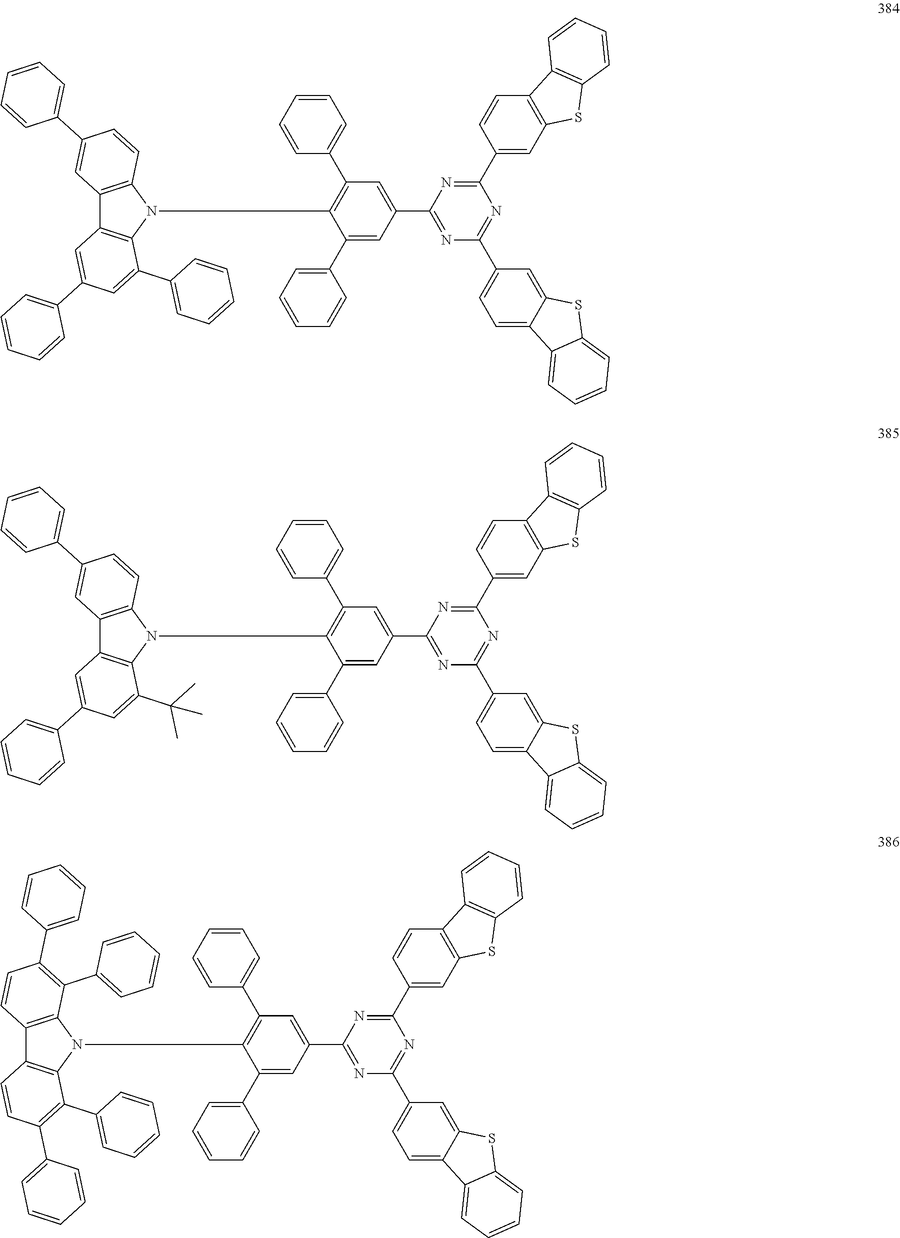

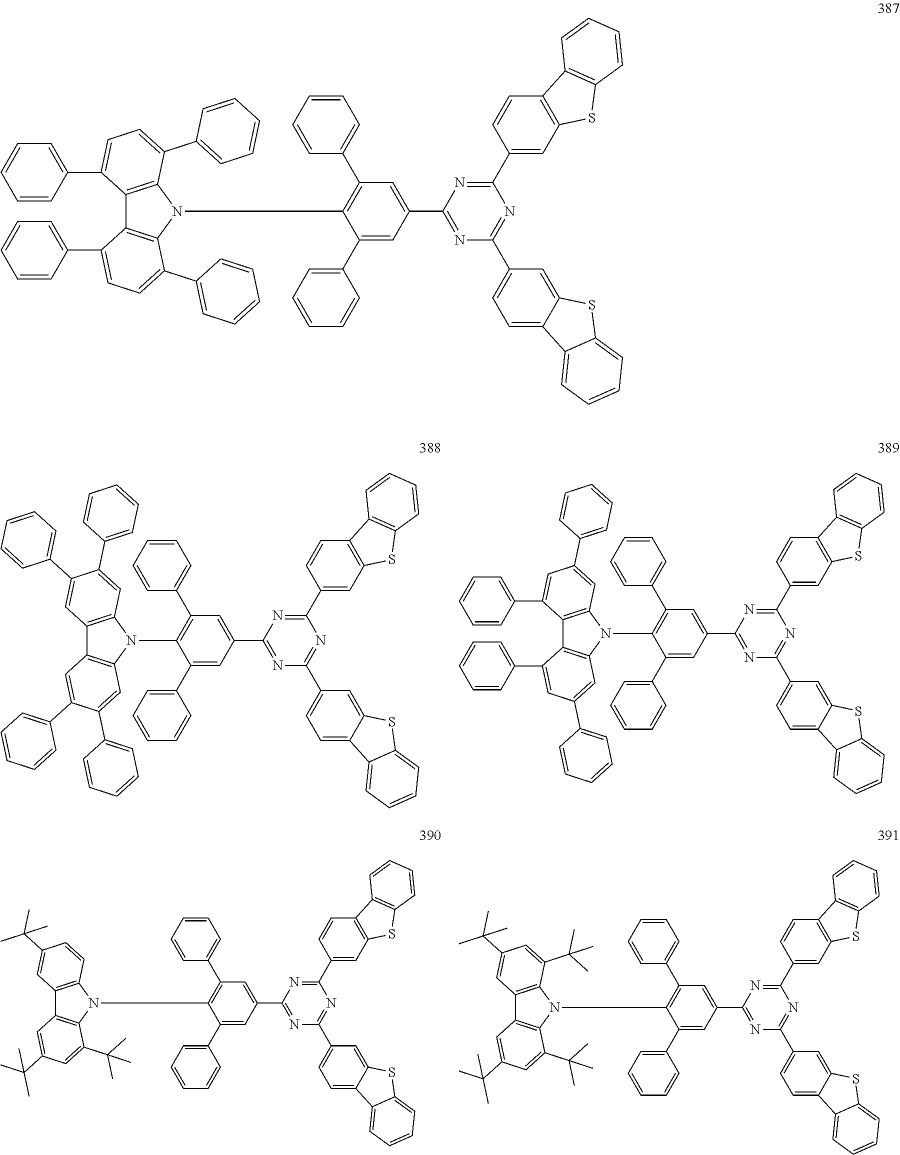

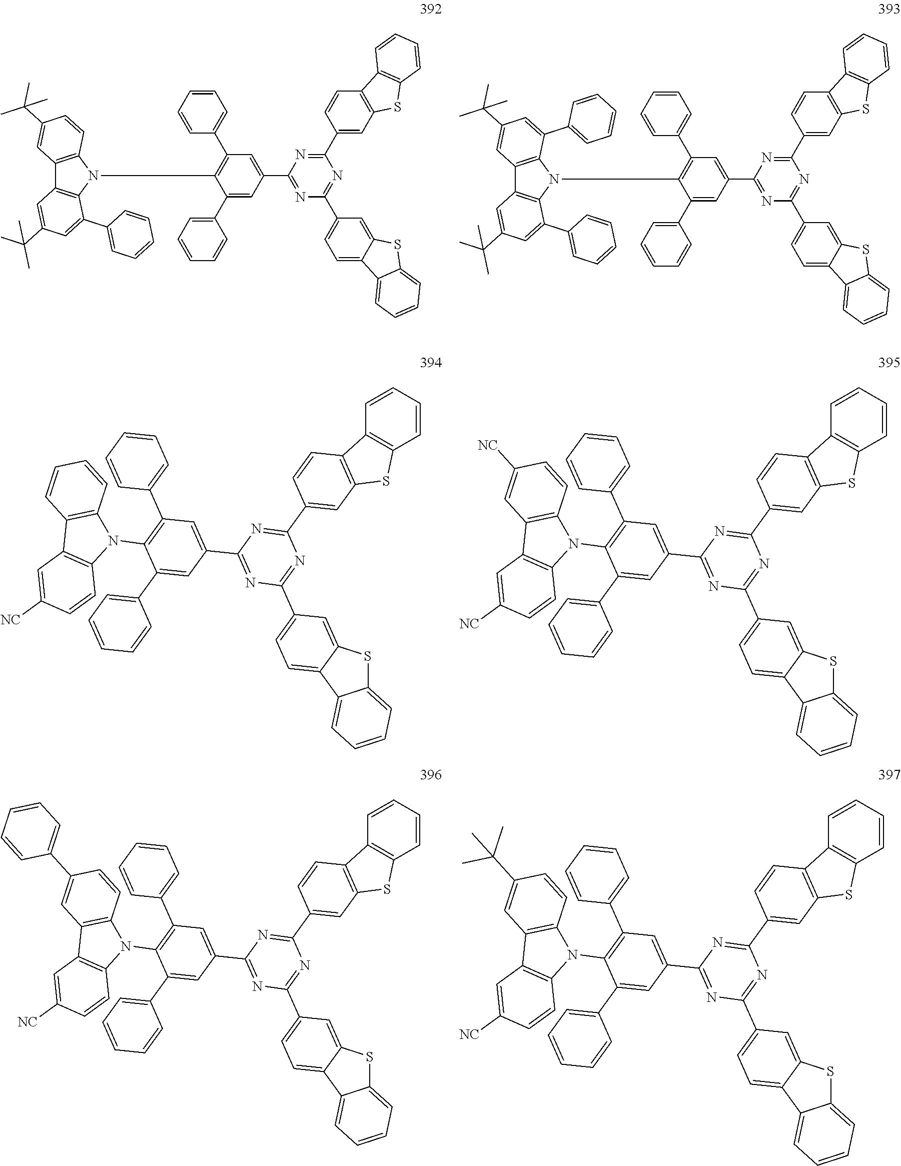

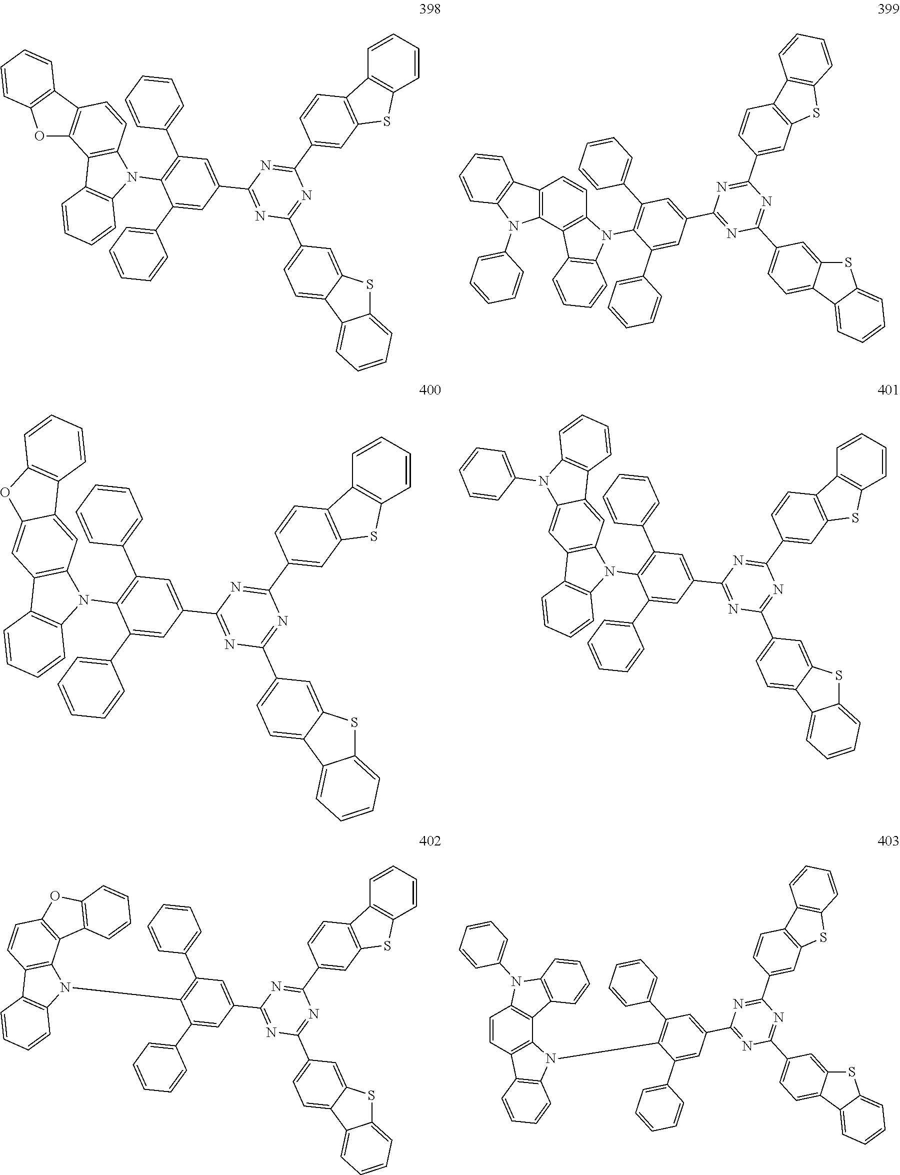

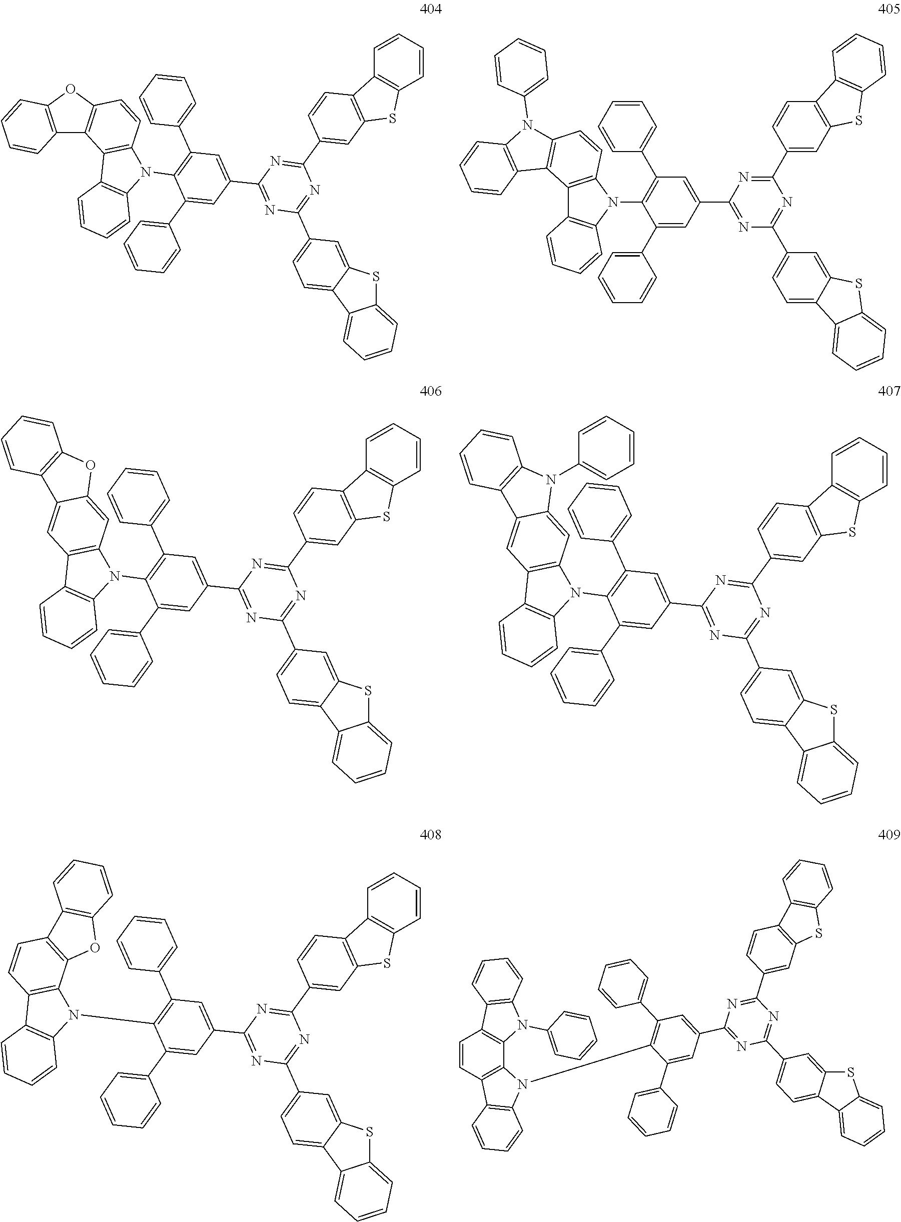

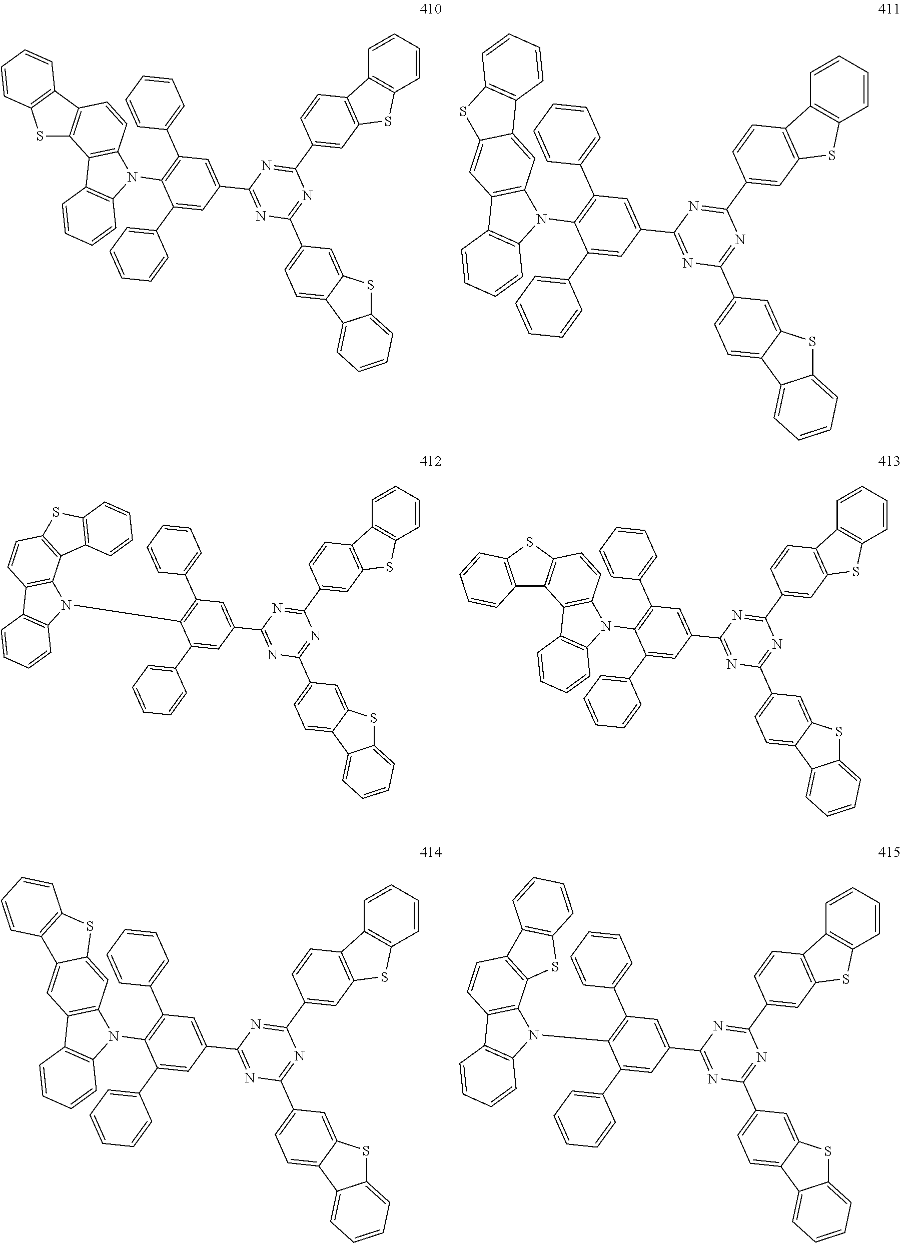

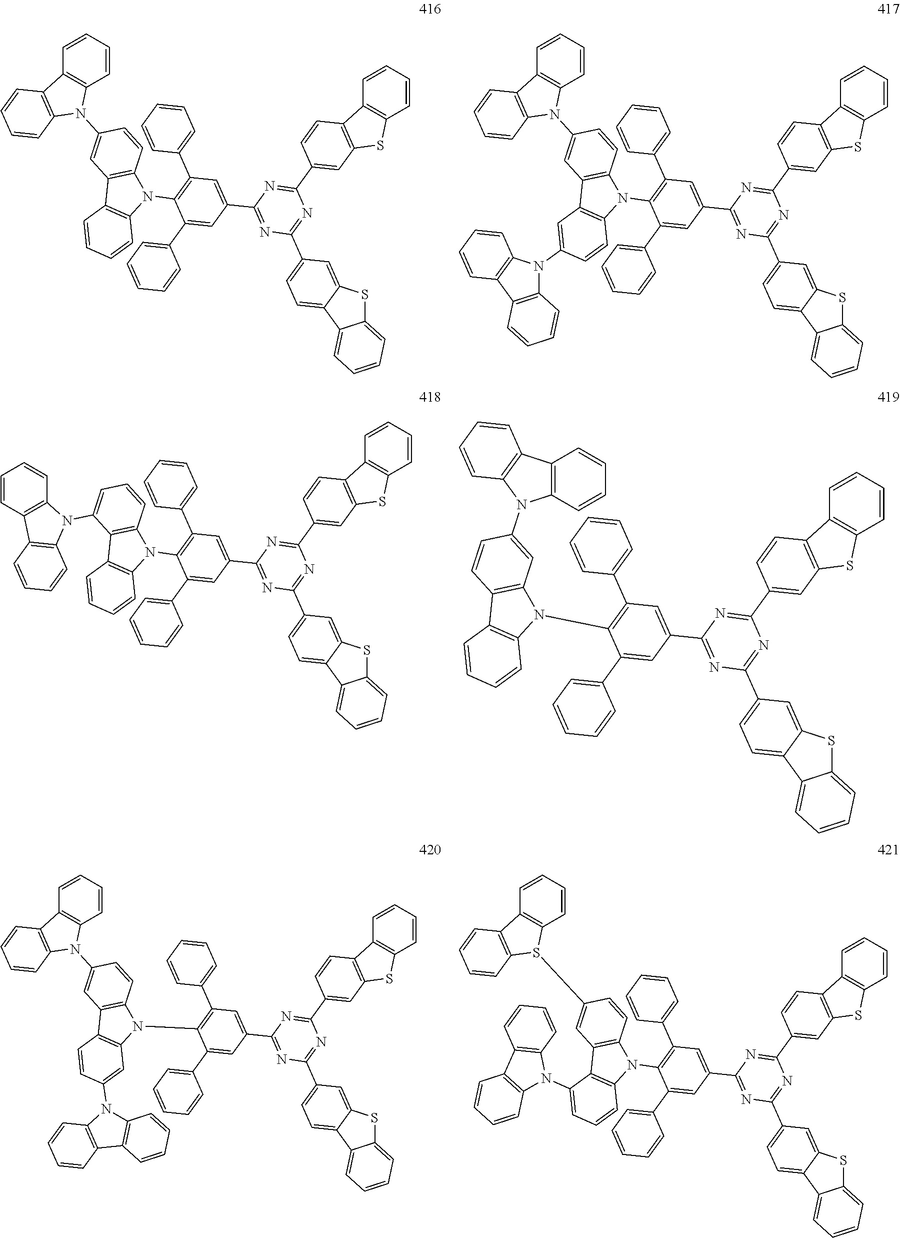

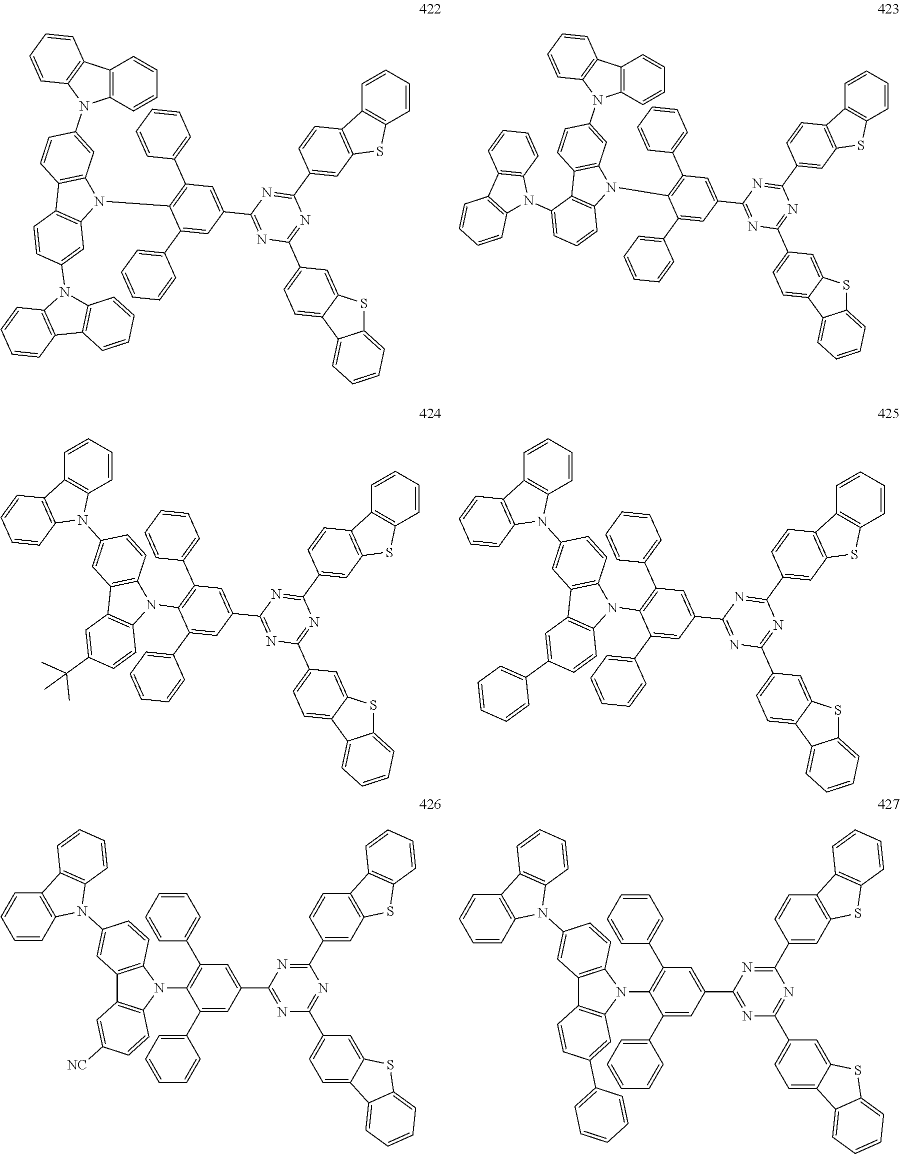

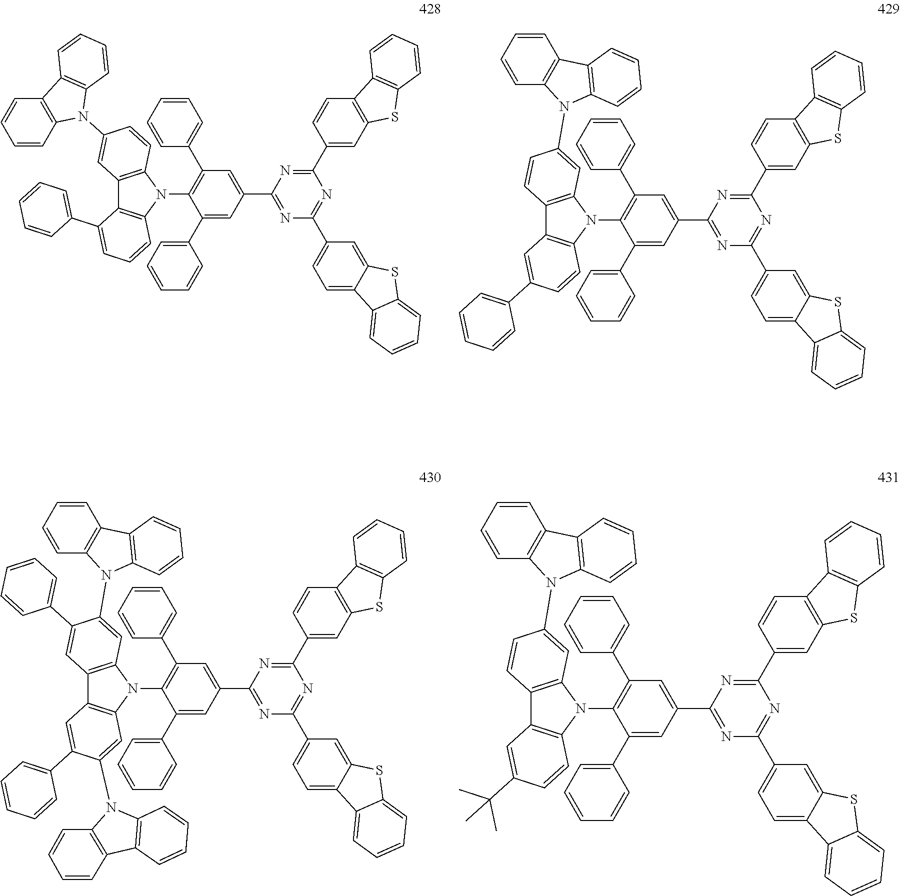

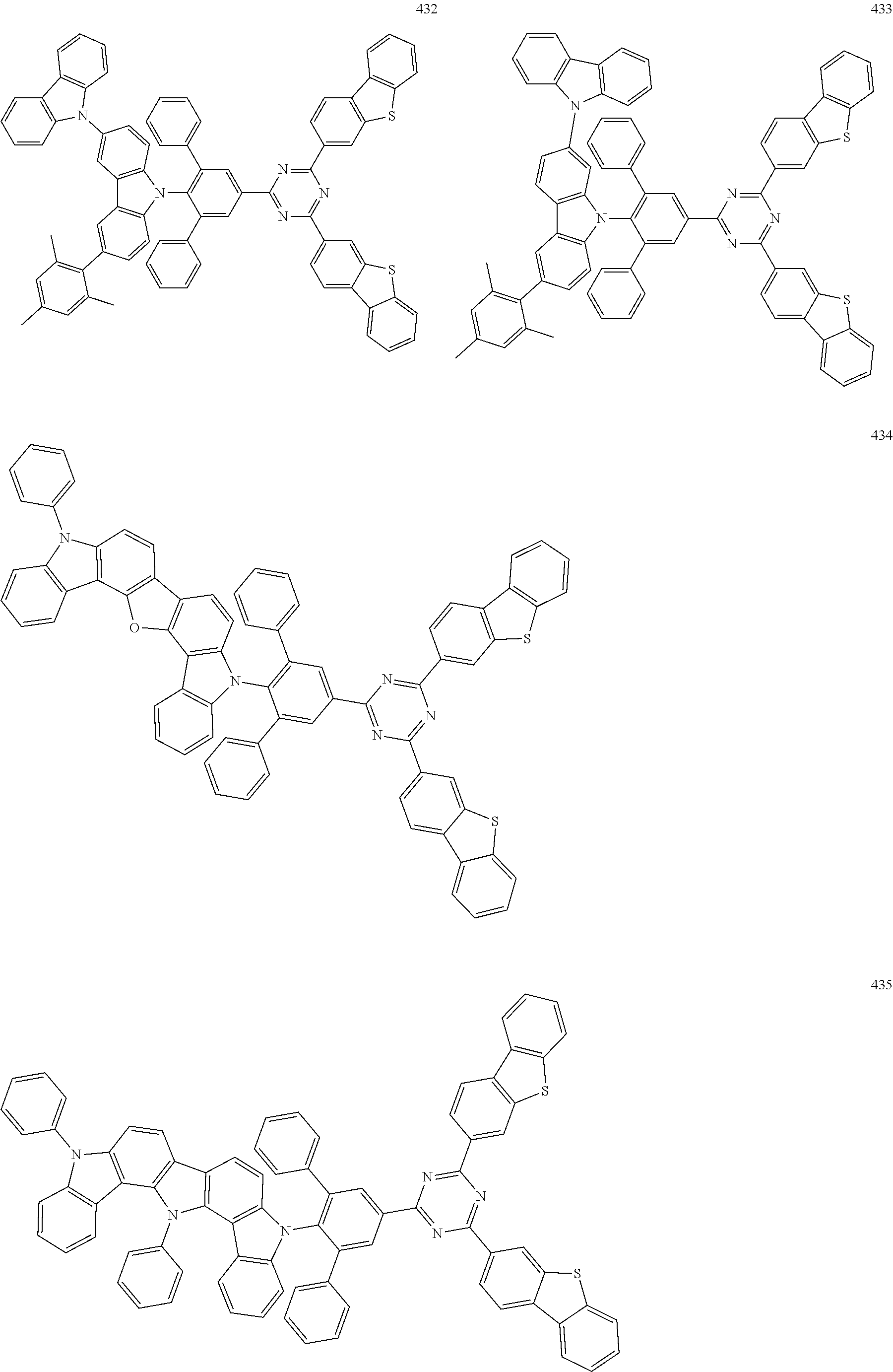

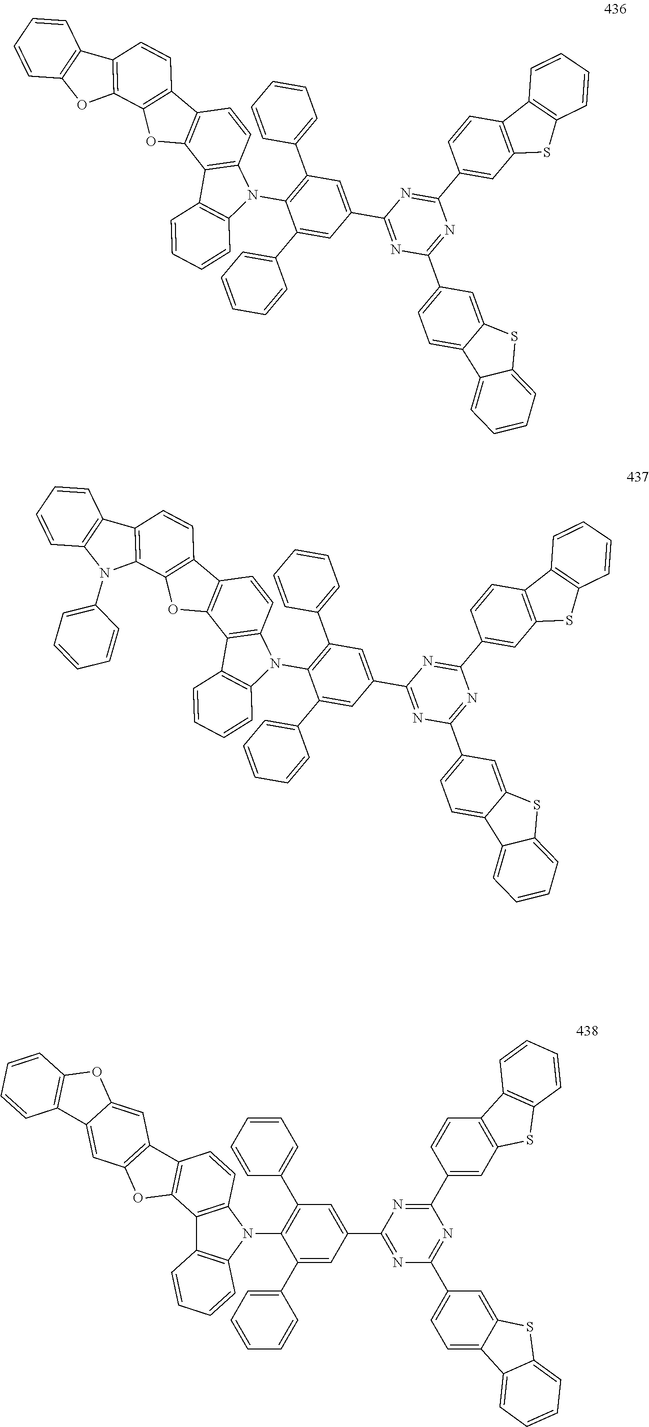 [SMILES: c1ccc(-c2cc(-c3nc(-c4ccc5c(c4)sc4ccccc45)nc(-c4ccc5c(c4)sc4ccccc45)n3)cc(-c3ccccc3)c2-n2c3ccccc3c3c4oc5c(ccc6c7ccccc7oc65)c4ccc32)cc1 ; c1ccc(-c2cc(-c3nc(-c4ccc5c(c4)sc4ccccc45)nc(-c4ccc5c(c4)sc4ccccc45)n3)cc(-c3ccccc3)c2-n2c3ccccc3c3c4oc5c(ccc6c7ccccc7n(-c7ccccc7)c65)c4ccc32)cc1 ; c1ccc(-c2cc(-c3nc(-c4ccc5c(c4)sc4ccccc45)nc(-c4ccc5c(c4)sc4ccccc45)n3)cc(-c3ccccc3)c2-n2c3ccccc3c3c4oc5cc6c(cc5c4ccc32)oc2ccccc26)cc1]

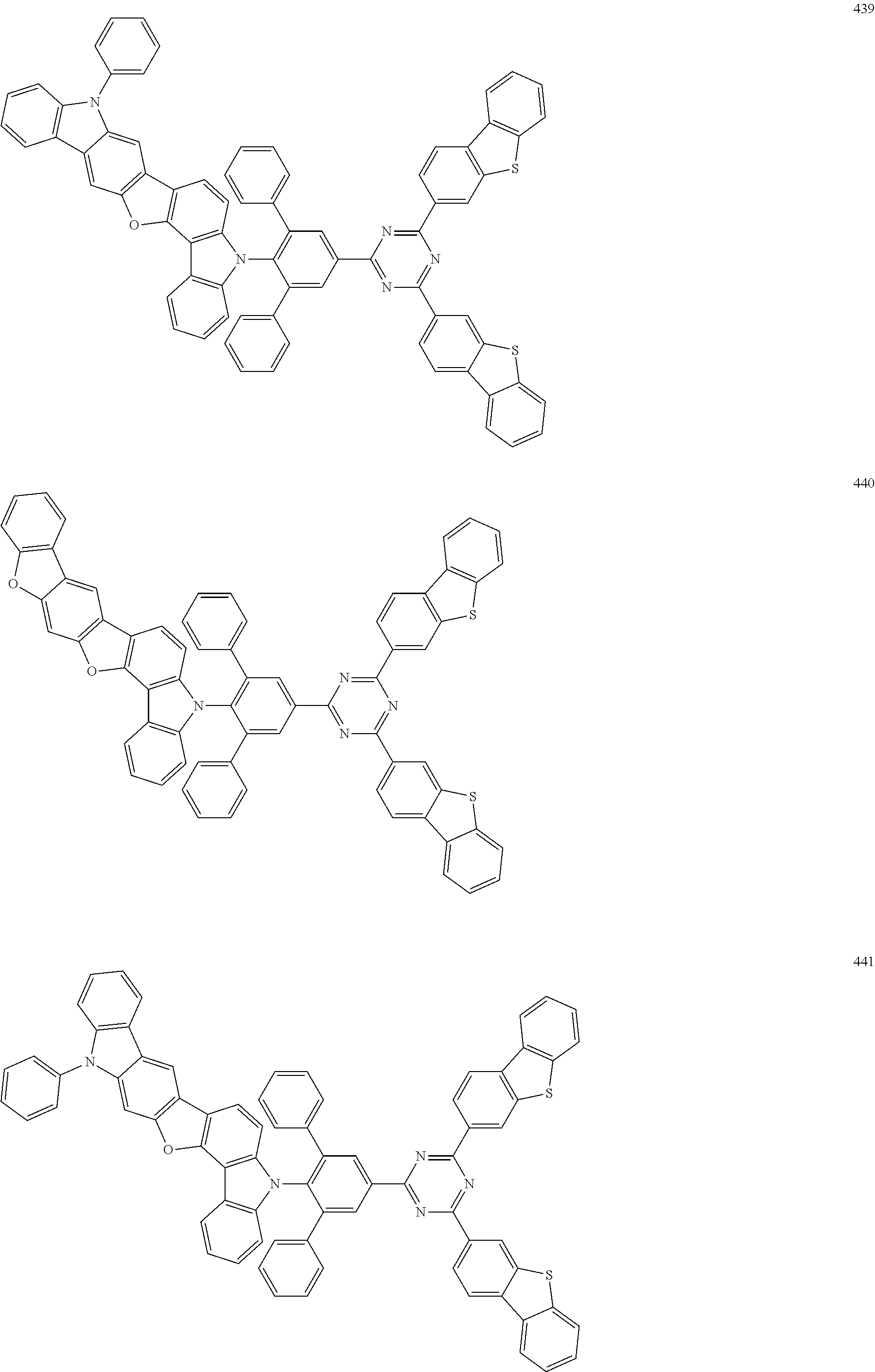 [SMILES: c1ccc(-c2cc(-c3nc(-c4ccc5c(c4)sc4ccccc45)nc(-c4ccc5c(c4)sc4ccccc45)n3)cc(-c3ccccc3)c2-n2c3ccccc3c3c4oc5cc6c7ccccc7n(-c7ccccc7)c6cc5c4ccc32)cc1 ; c1ccc(-c2cc(-c3nc(-c4ccc5c(c4)sc4ccccc45)nc(-c4ccc5c(c4)sc4ccccc45)n3)cc(-c3ccccc3)c2-n2c3ccccc3c3c4oc5cc6oc7ccccc7c6cc5c4ccc32)cc1 ; c1ccc(-c2cc(-c3nc(-c4ccc5c(c4)sc4ccccc45)nc(-c4ccc5c(c4)sc4ccccc45)n3)cc(-c3ccccc3)c2-n2c3ccccc3c3c4oc5cc6c(cc5c4ccc32)c2ccccc2n6-c2ccccc2)cc1]

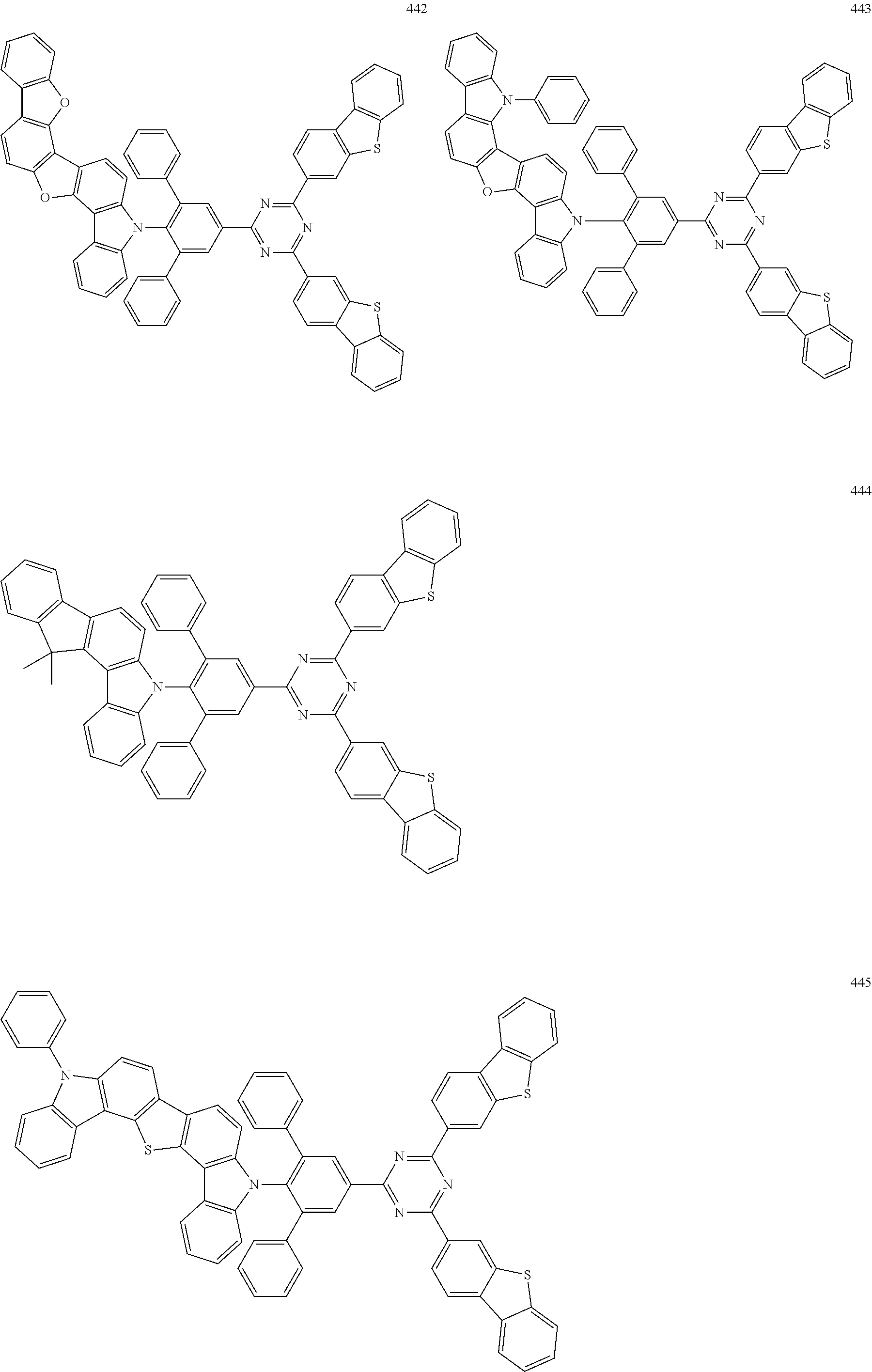

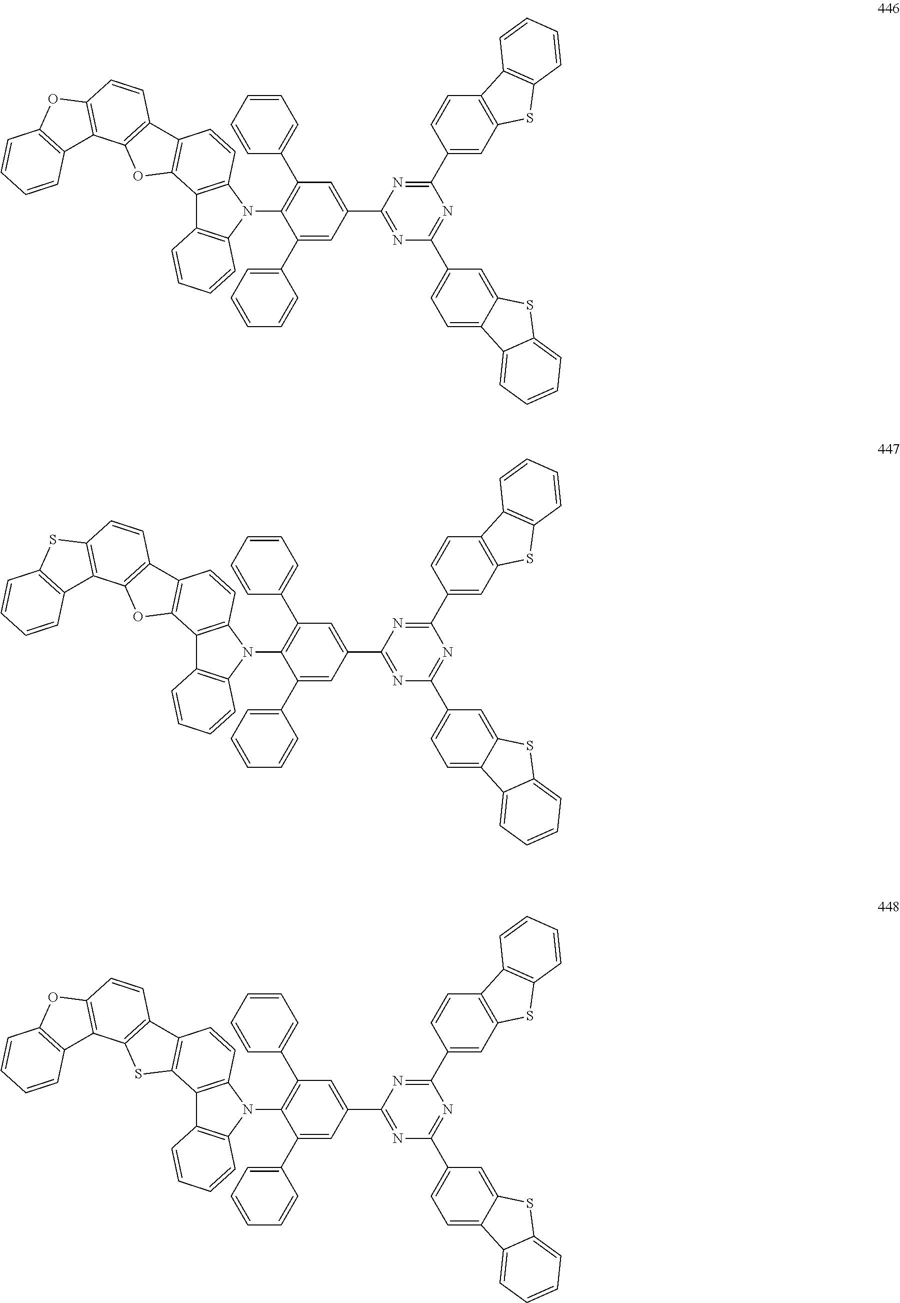 [SMILES: c1ccc(-c2cc(-c3nc(-c4ccc5c(c4)sc4ccccc45)nc(-c4ccc5c(c4)sc4ccccc45)n3)cc(-c3ccccc3)c2-n2c3ccccc3c3c4oc5c(ccc6oc7ccccc7c65)c4ccc32)cc1 ; c1ccc(-c2cc(-c3nc(-c4ccc5c(c4)sc4ccccc45)nc(-c4ccc5c(c4)sc4ccccc45)n3)cc(-c3ccccc3)c2-n2c3ccccc3c3c4oc5c(ccc6sc7ccccc7c65)c4ccc32)cc1 ; c1ccc(-c2cc(-c3nc(-c4ccc5c(c4)sc4ccccc45)nc(-c4ccc5c(c4)sc4ccccc45)n3)cc(-c3ccccc3)c2-n2c3ccccc3c3c4sc5c(ccc6oc7ccccc7c65)c4ccc32)cc1]

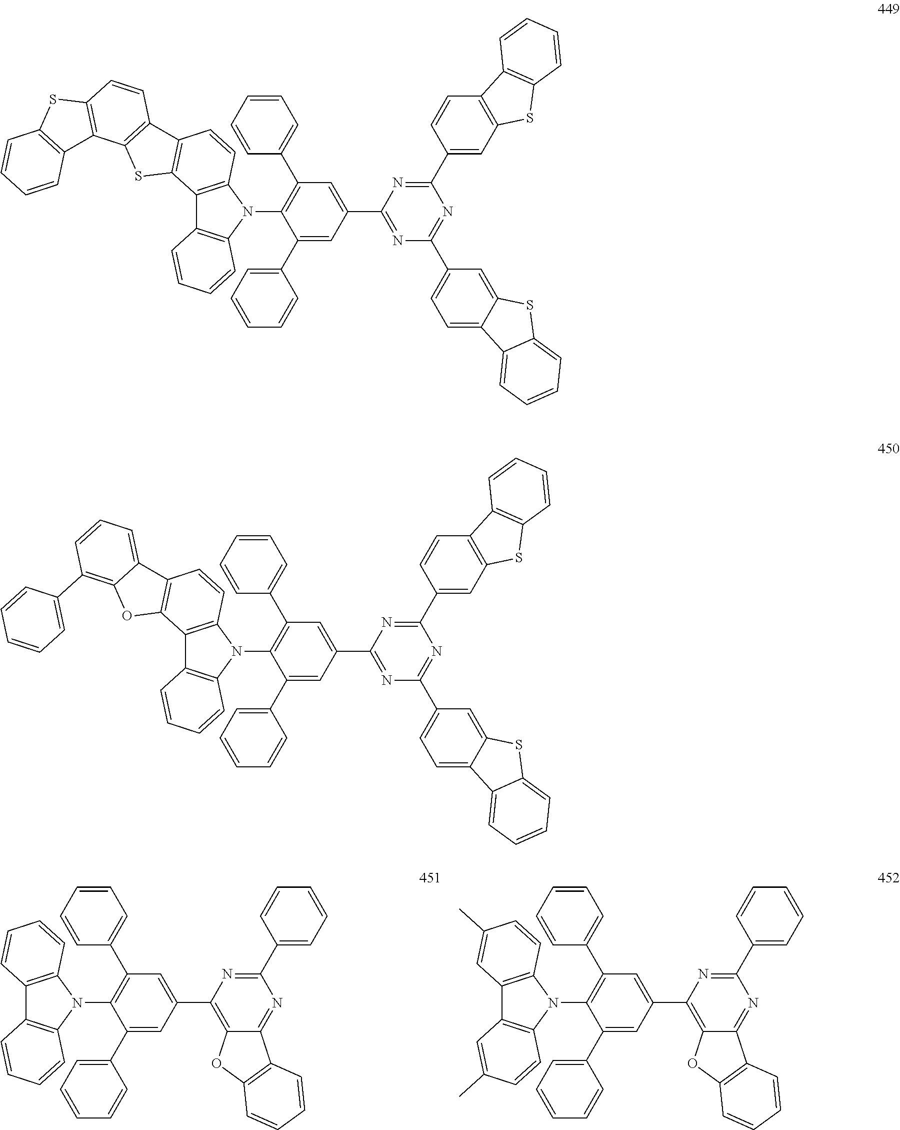 [SMILES: c1ccc(-c2cc(-c3nc(-c4ccc5c(c4)sc4ccccc45)nc(-c4ccc5c(c4)sc4ccccc45)n3)cc(-c3ccccc3)c2-n2c3ccccc3c3c4sc5c(ccc6sc7ccccc7c65)c4ccc32)cc1 ; c1ccc(-c2cc(-c3nc(-c4ccc5c(c4)sc4ccccc45)nc(-c4ccc5c(c4)sc4ccccc45)n3)cc(-c3ccccc3)c2-n2c3ccccc3c3c4oc5c(-c6ccccc6)cccc5c4ccc32)cc1 ; c1ccc(-c2nc(-c3cc(-c4ccccc4)c(-n4c5ccccc5c5ccccc54)c(-c4ccccc4)c3)c3oc4ccccc4c3n2)cc1 ; Cc1ccc2c(c1)c1cc(C)ccc1n2-c1c(-c2ccccc2)cc(-c2nc(-c3ccccc3)nc3c2oc2ccccc23)cc1-c1ccccc1]

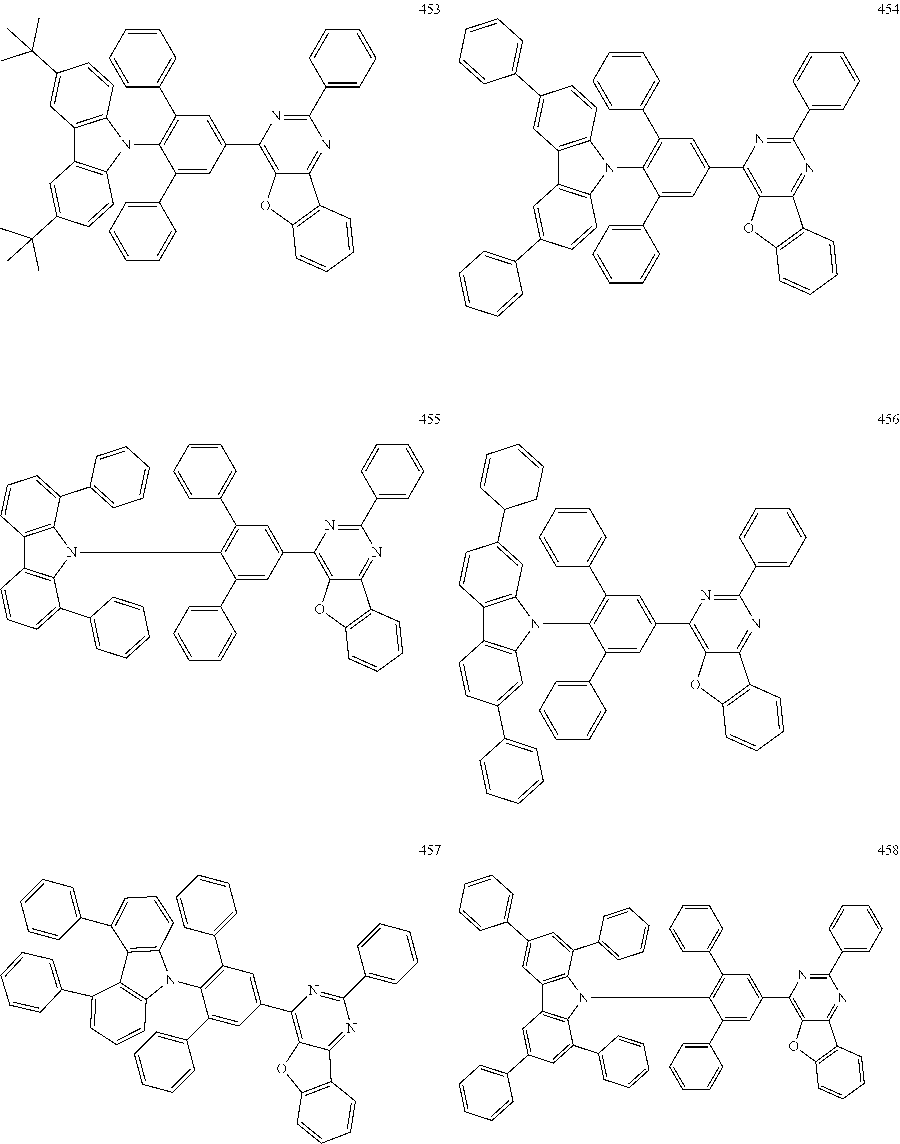

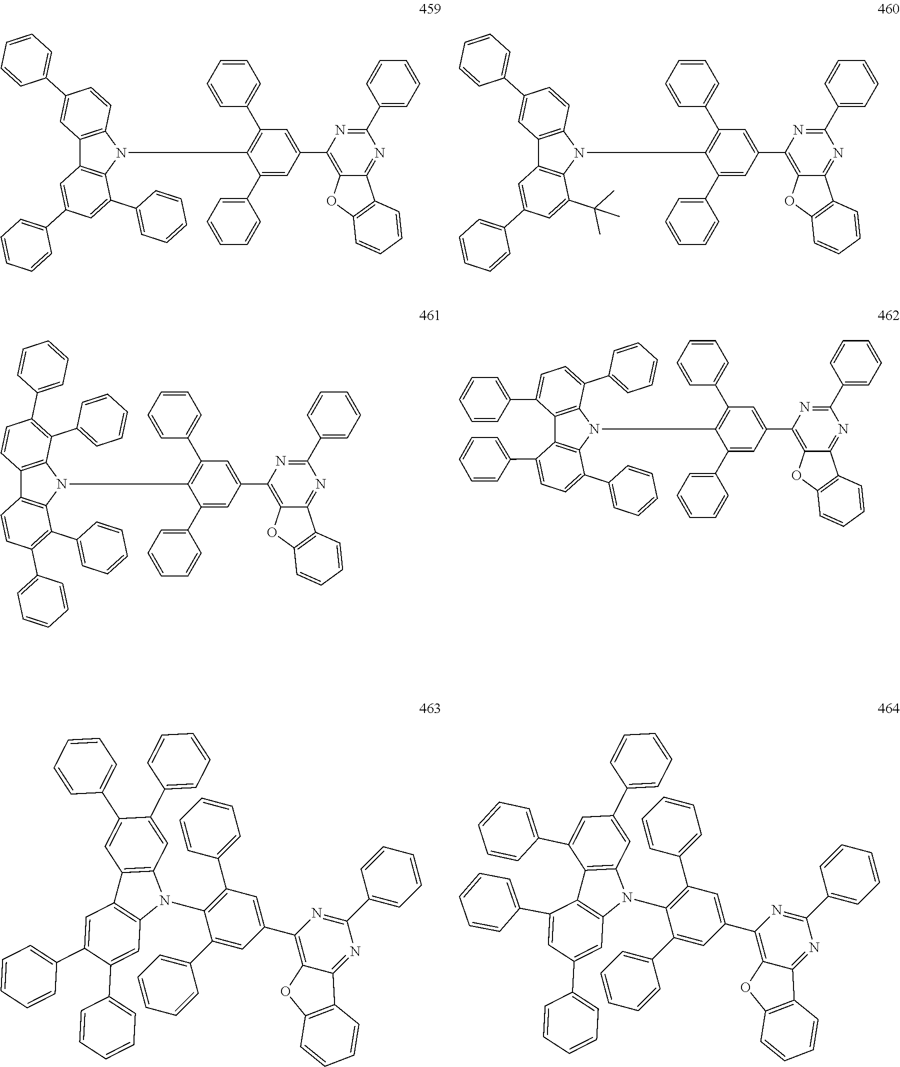

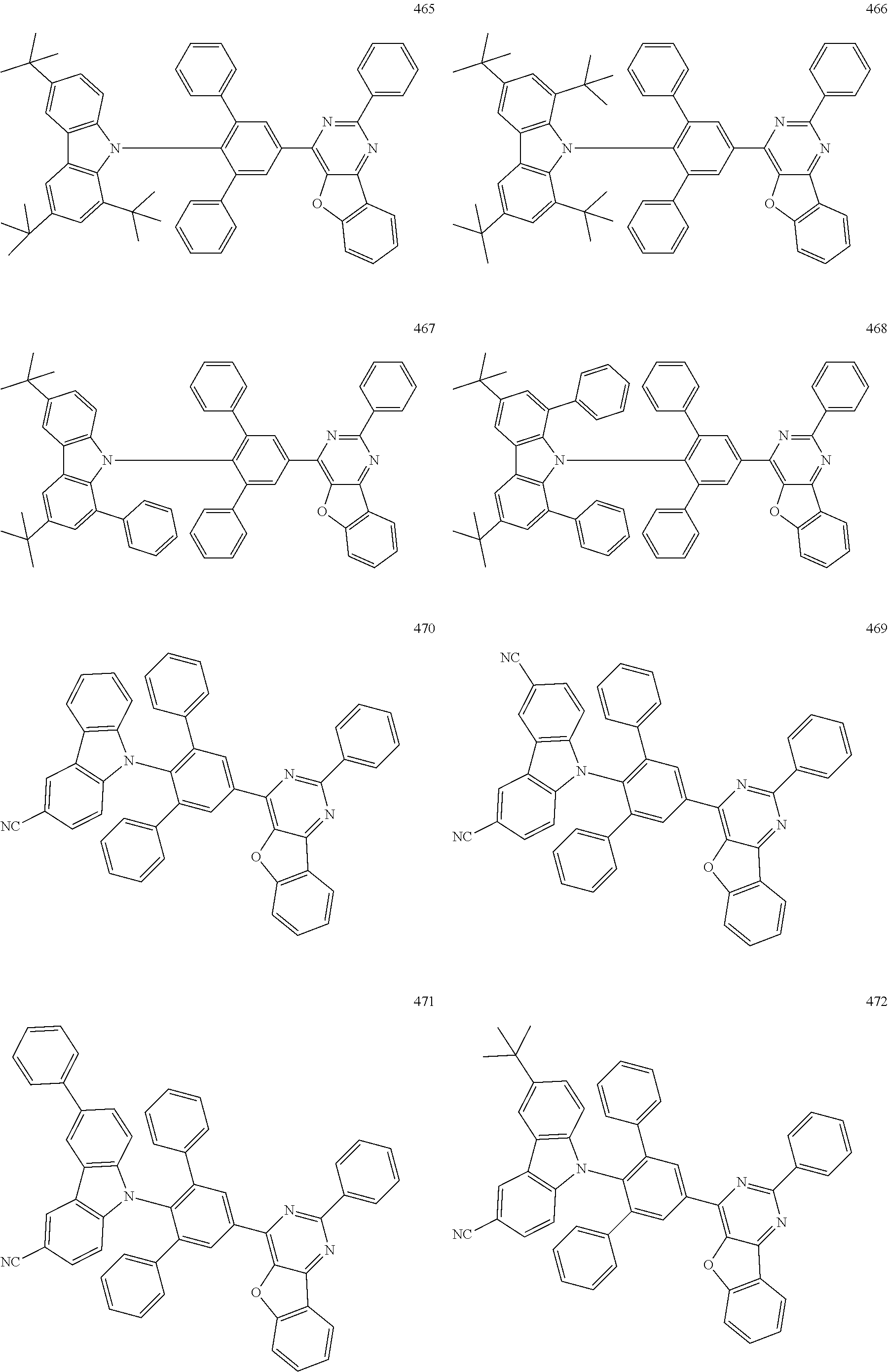

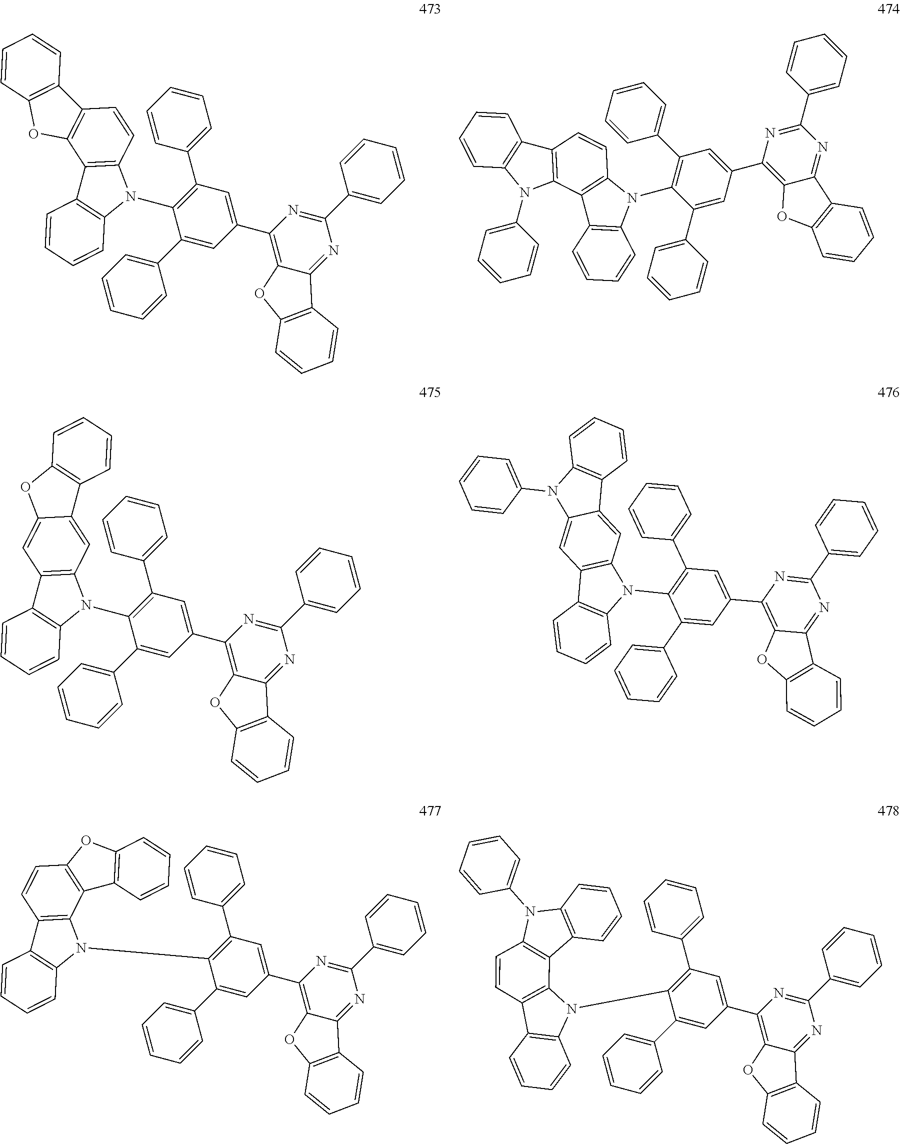

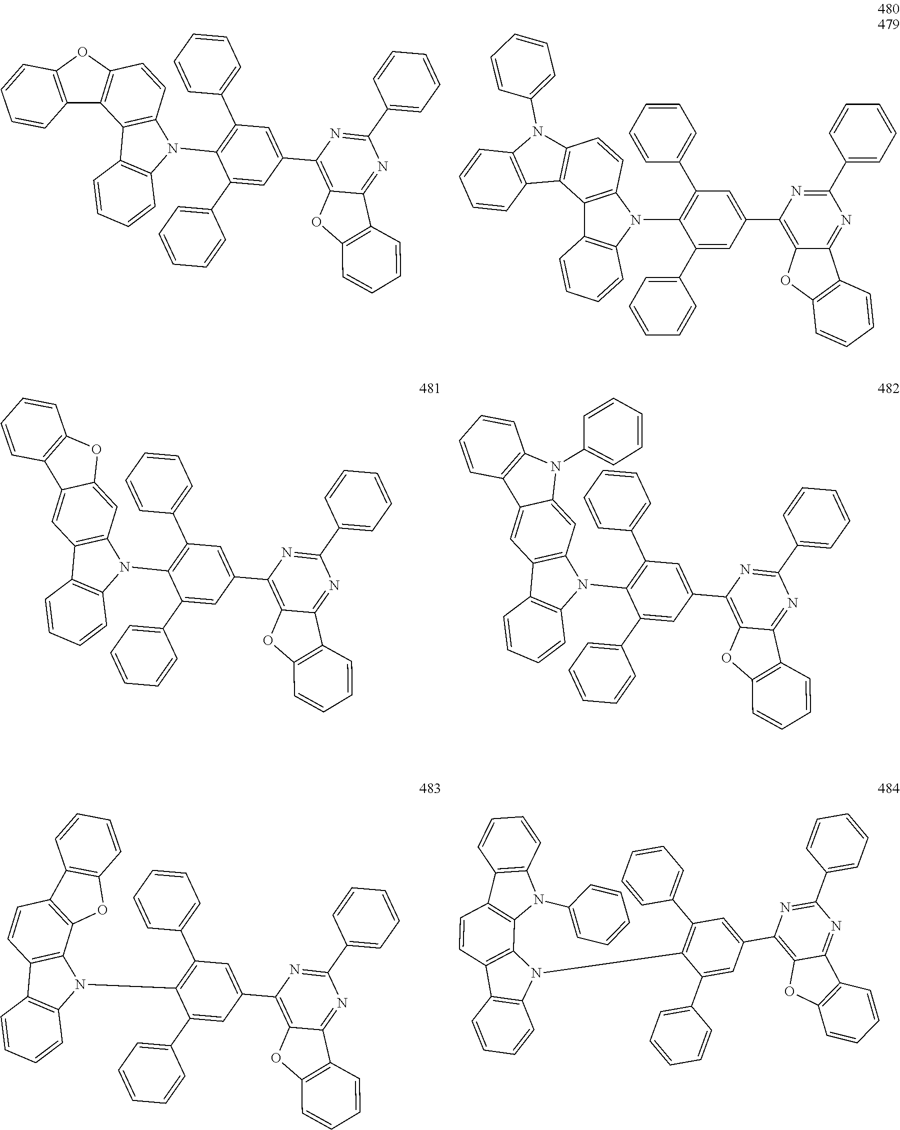

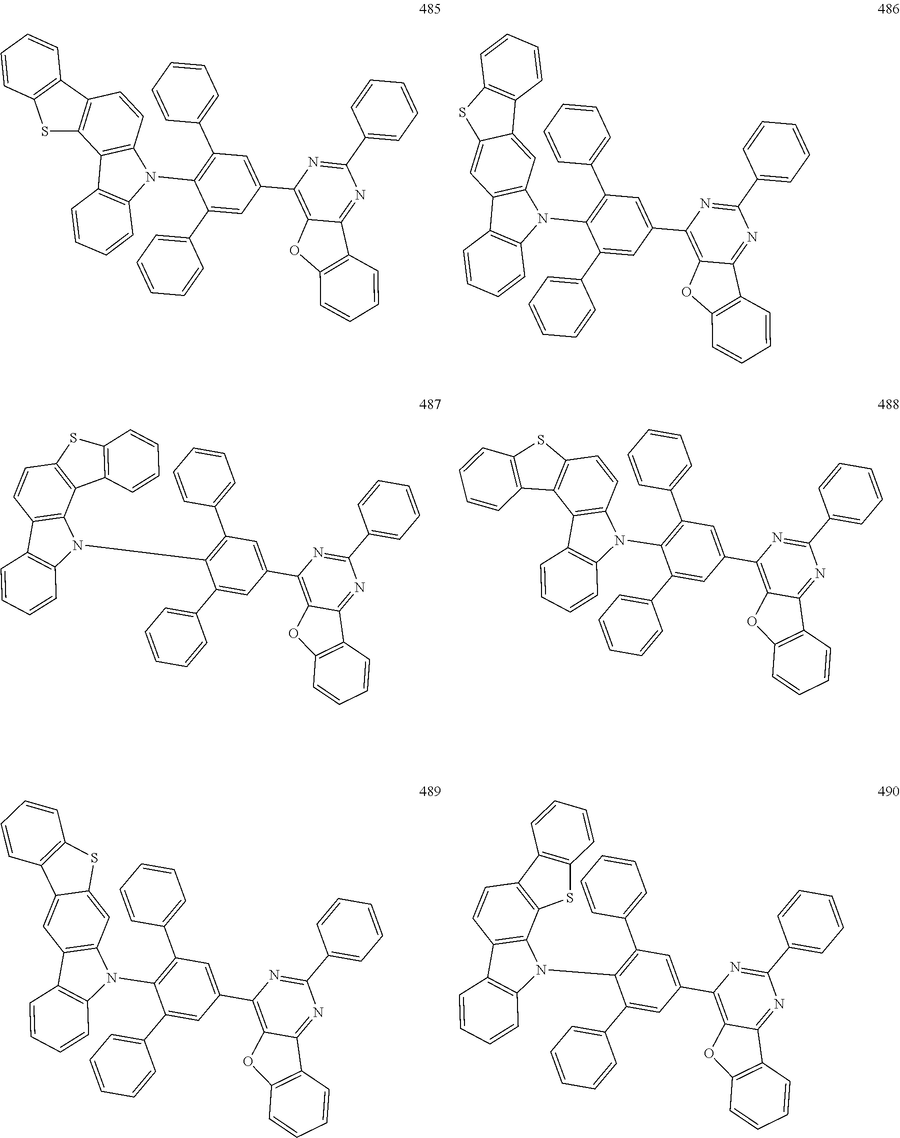

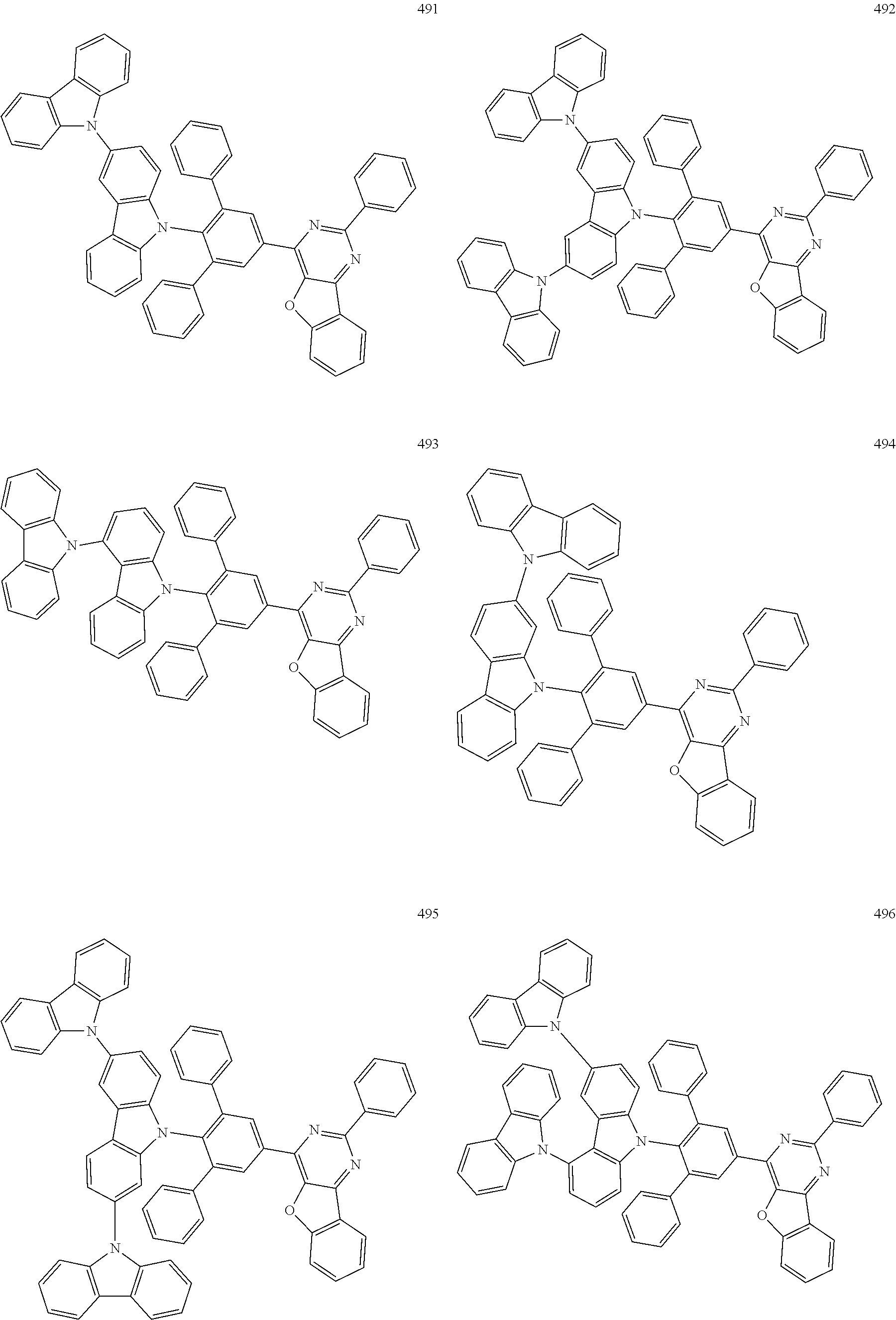

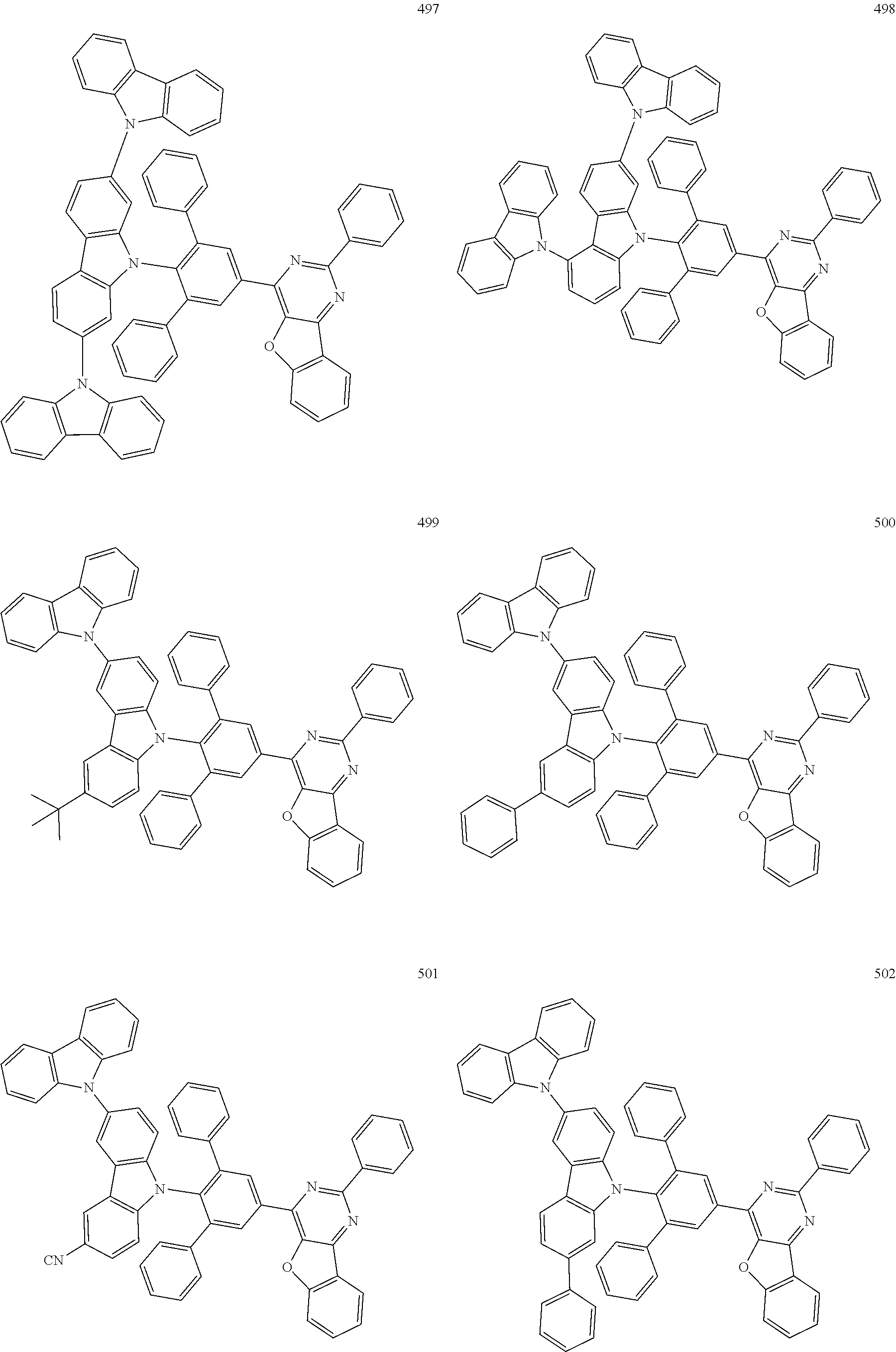

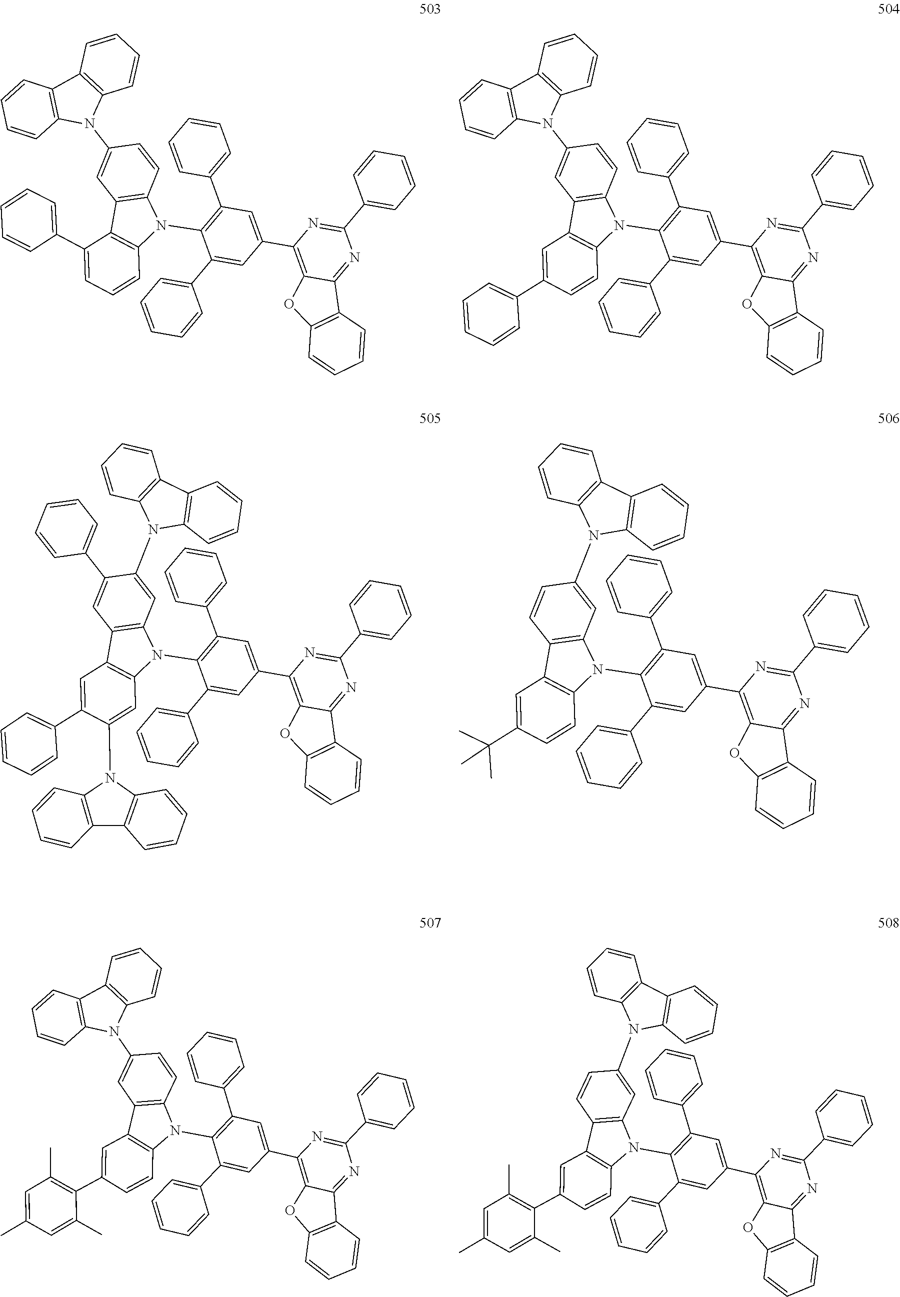

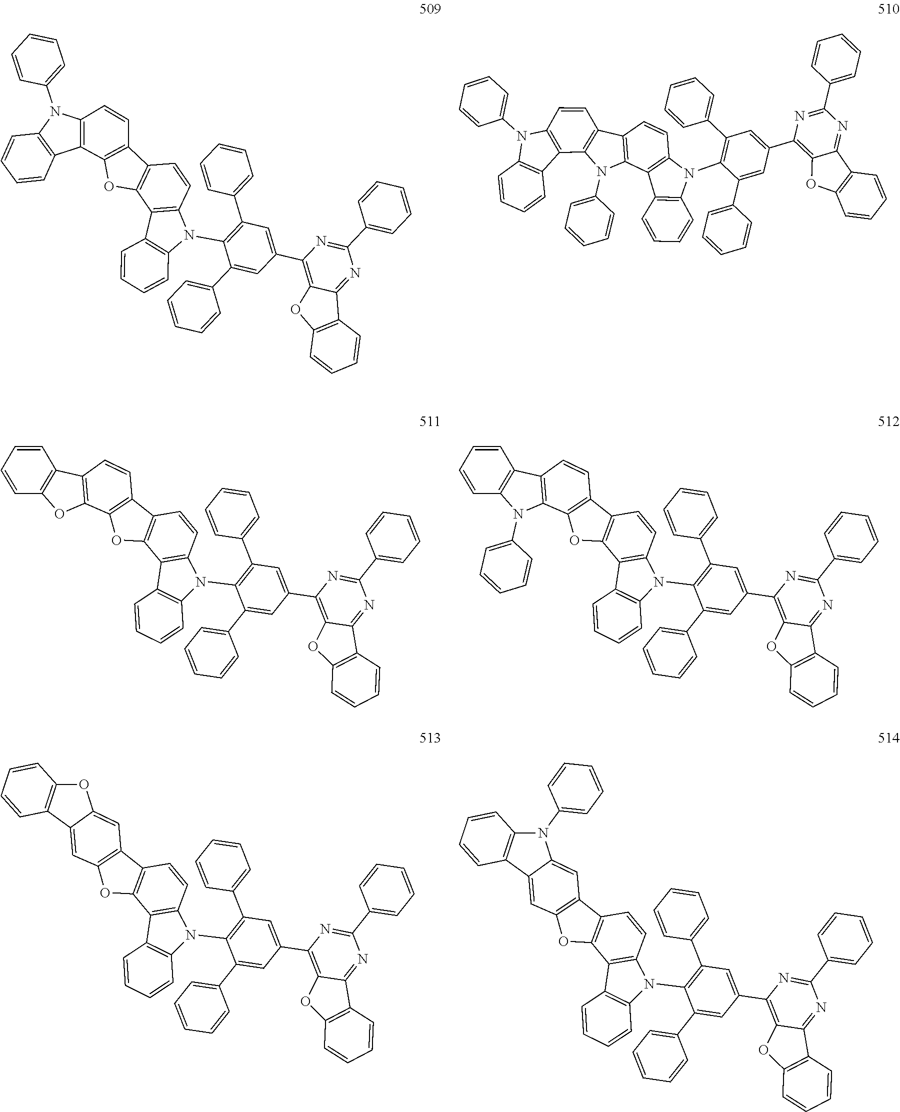

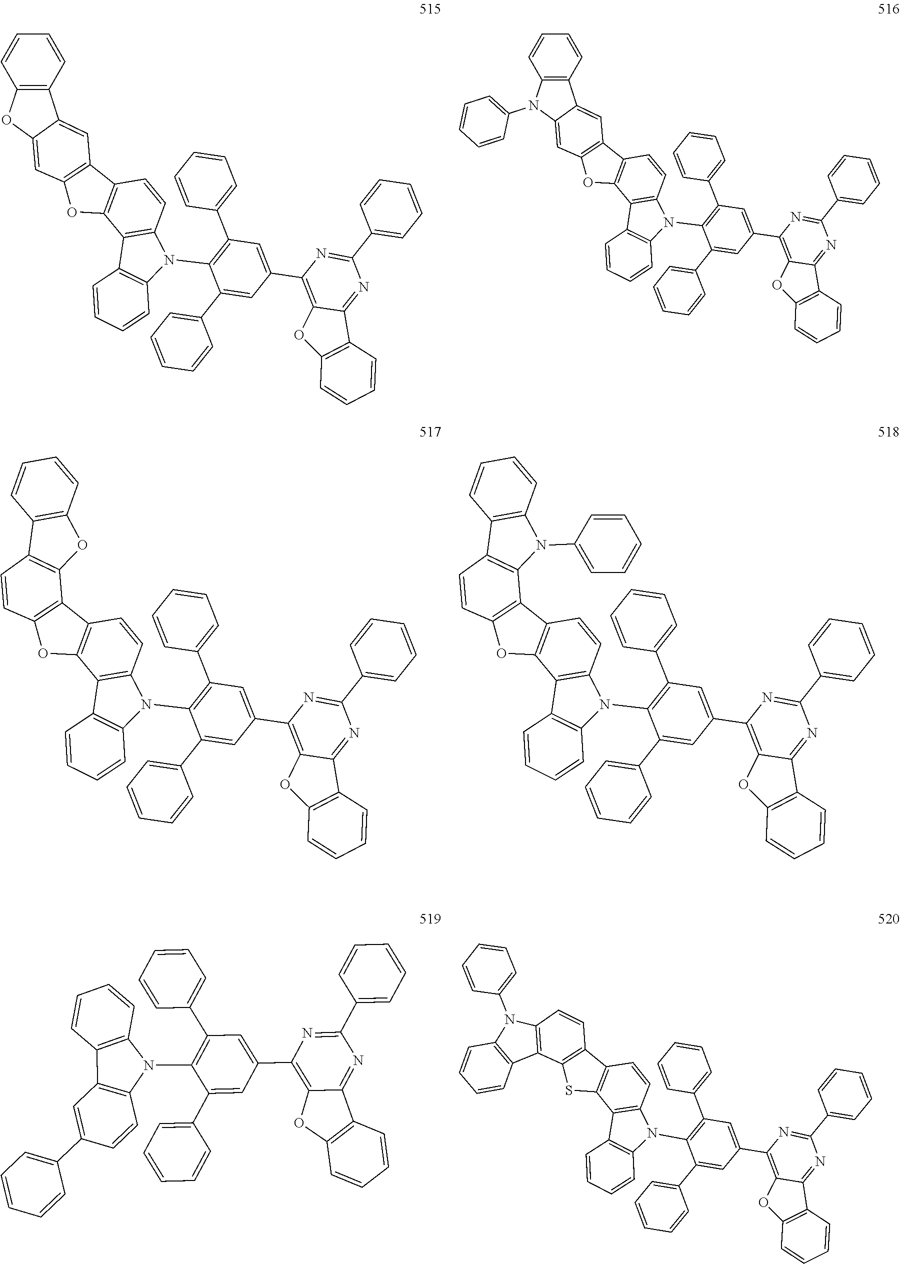

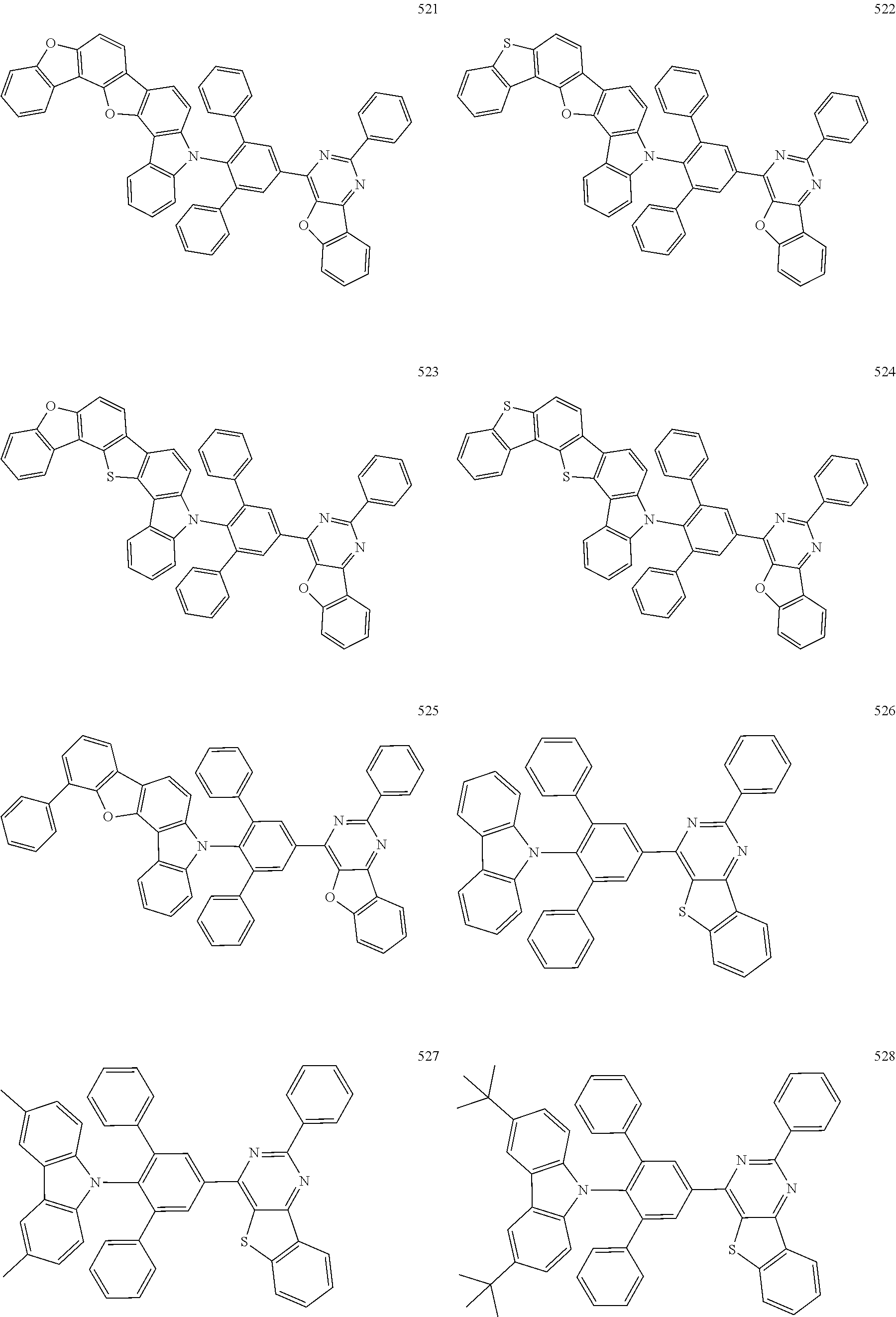

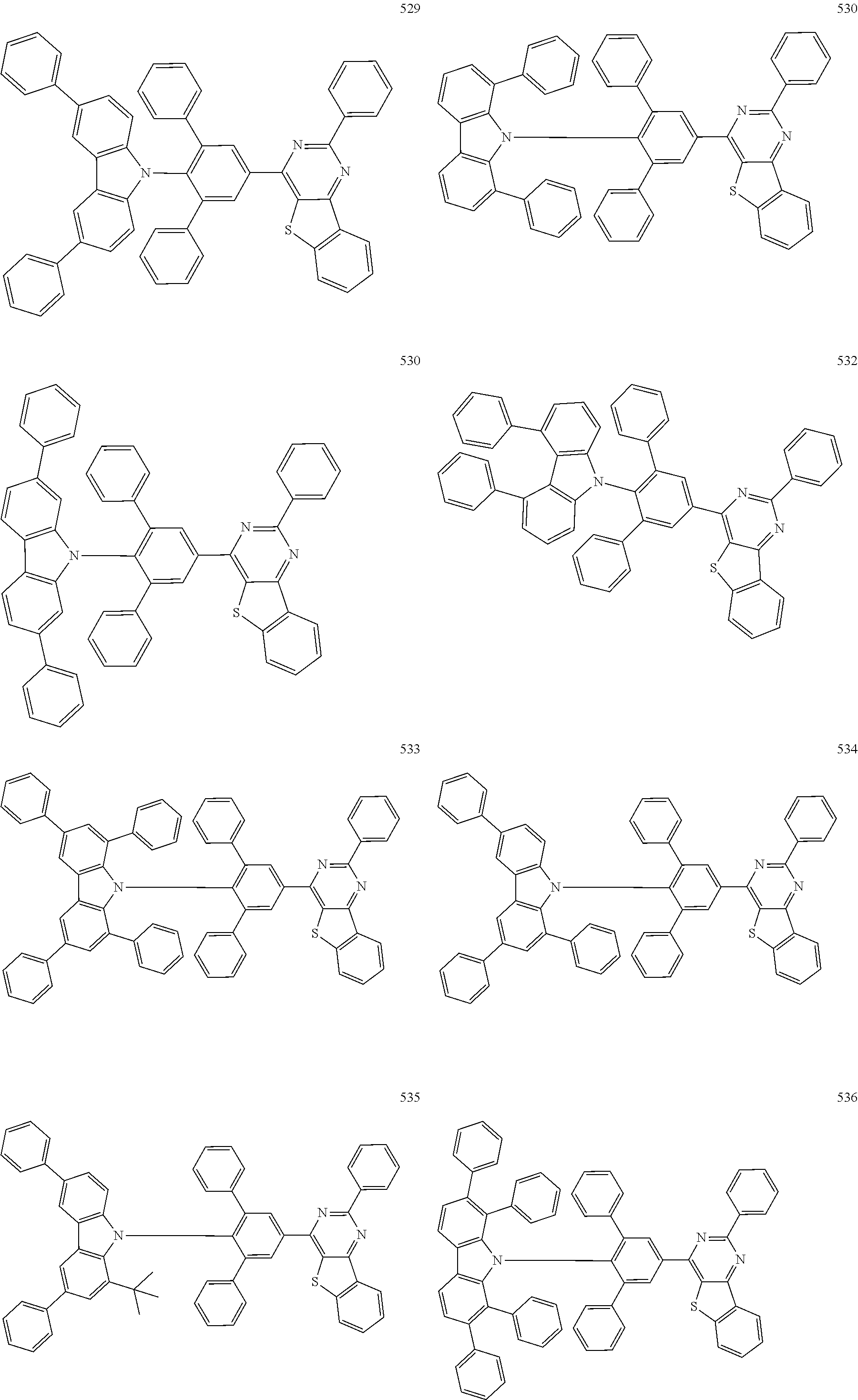

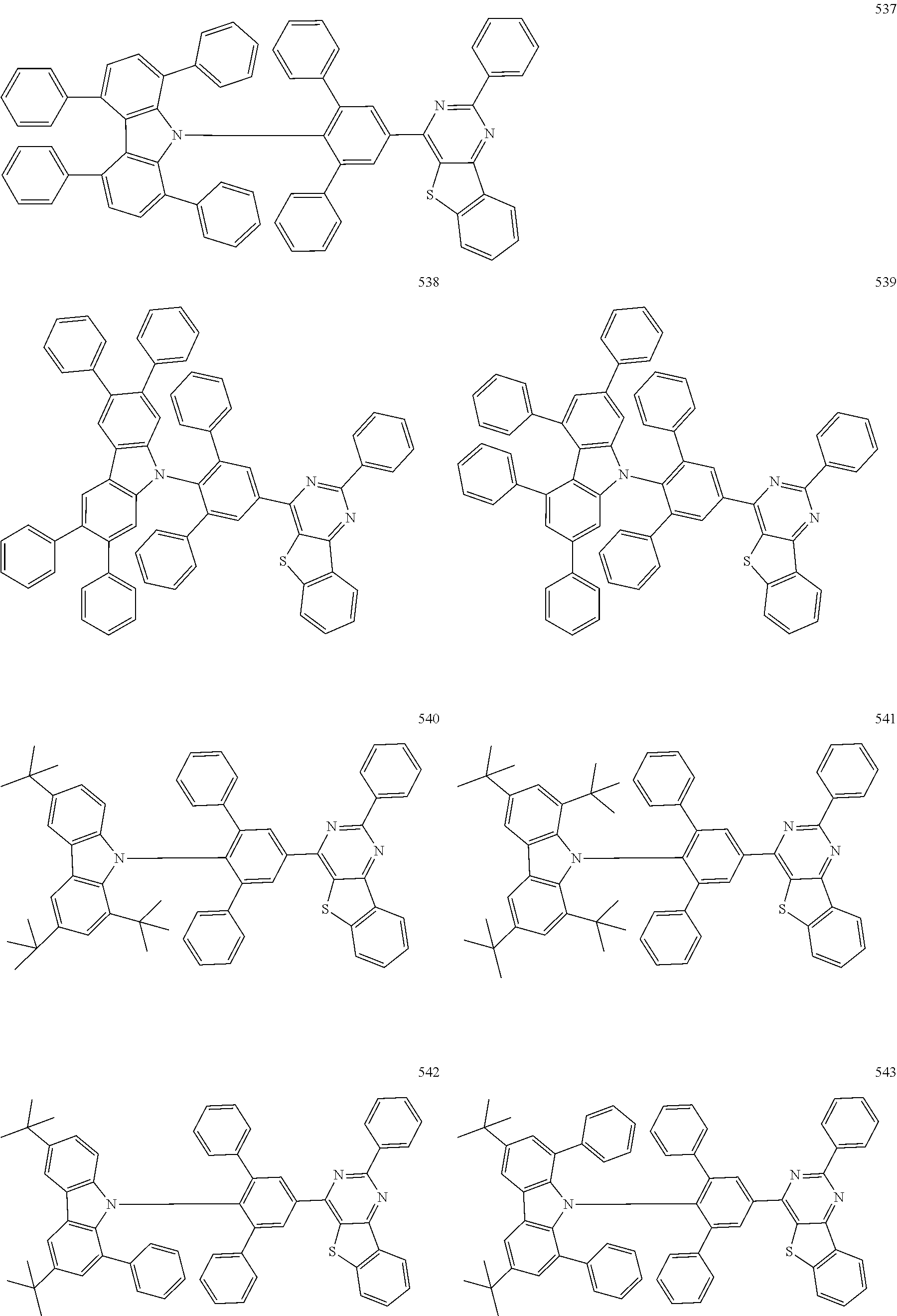

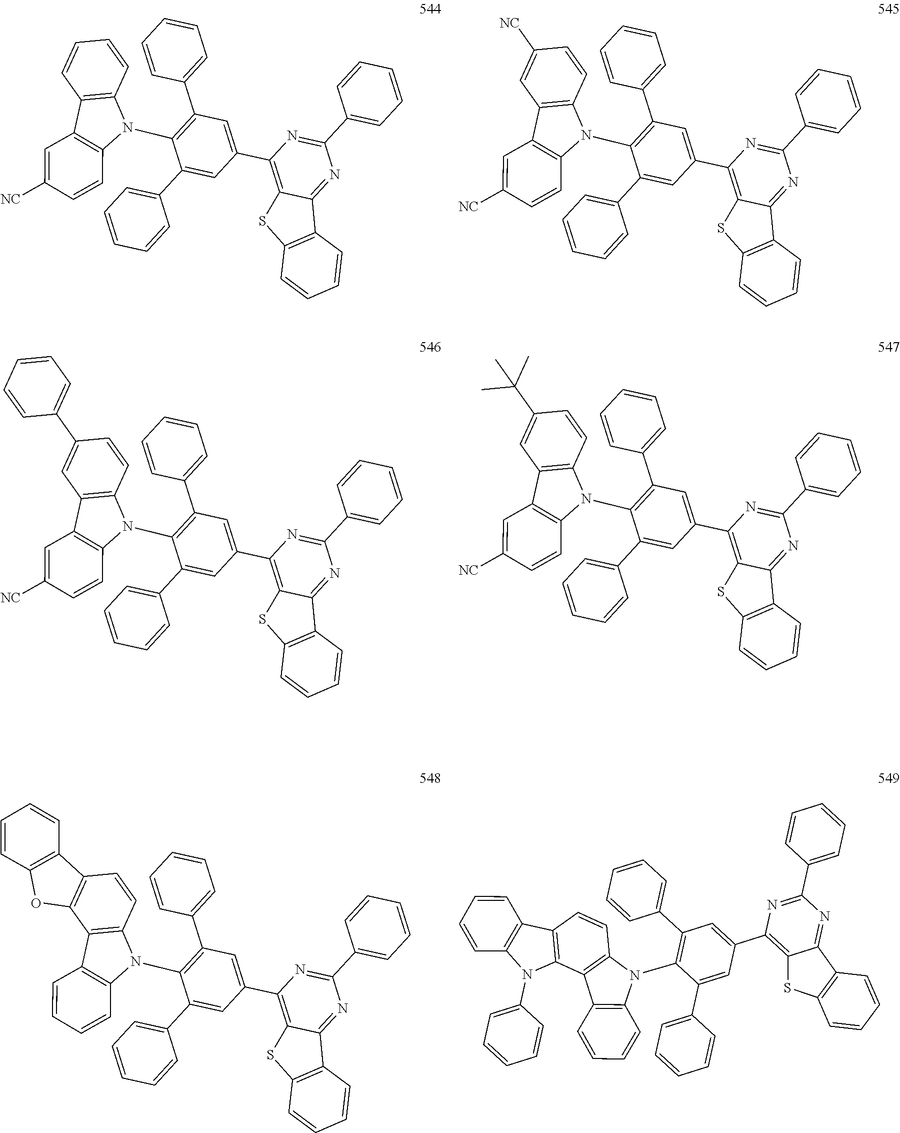

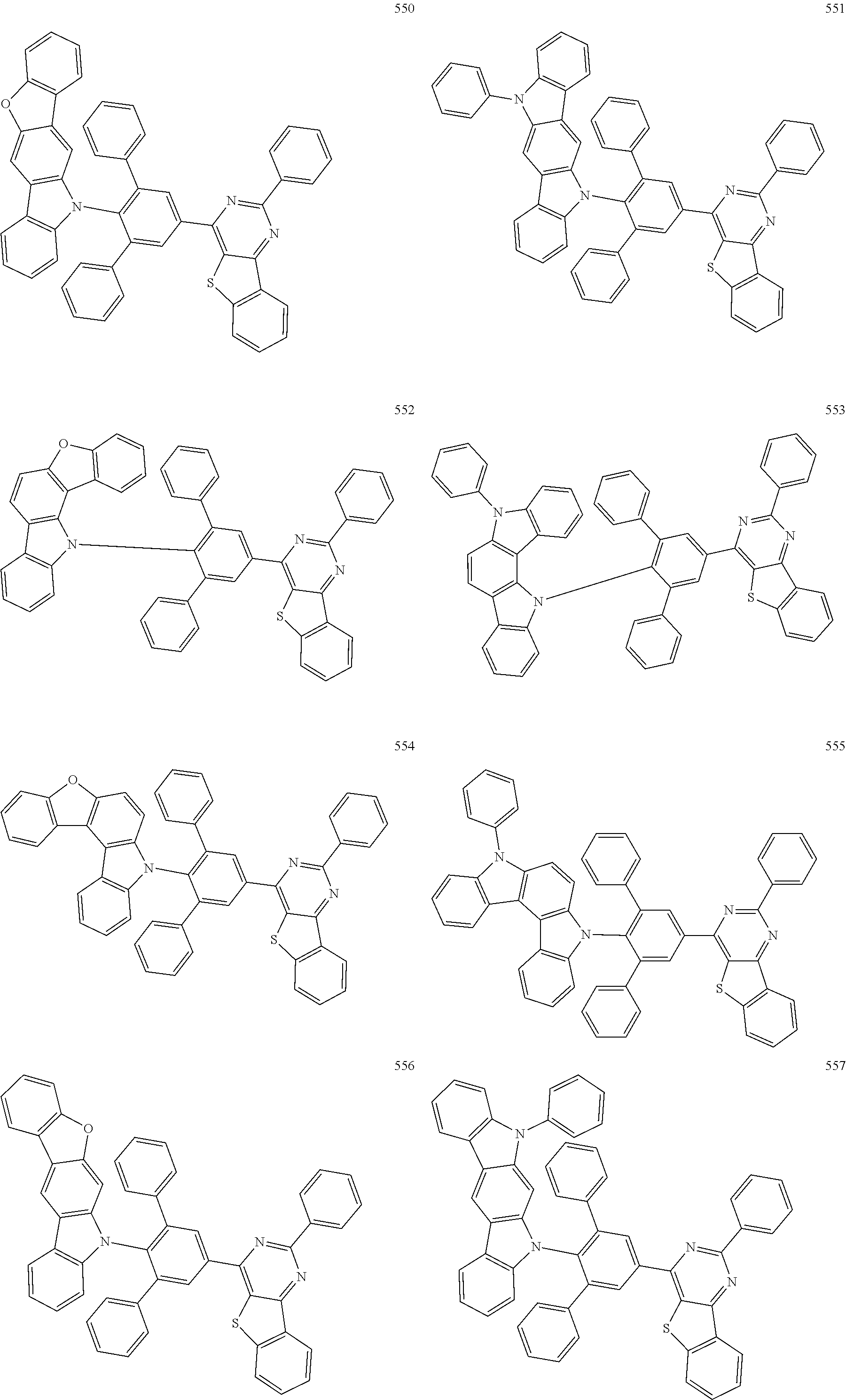

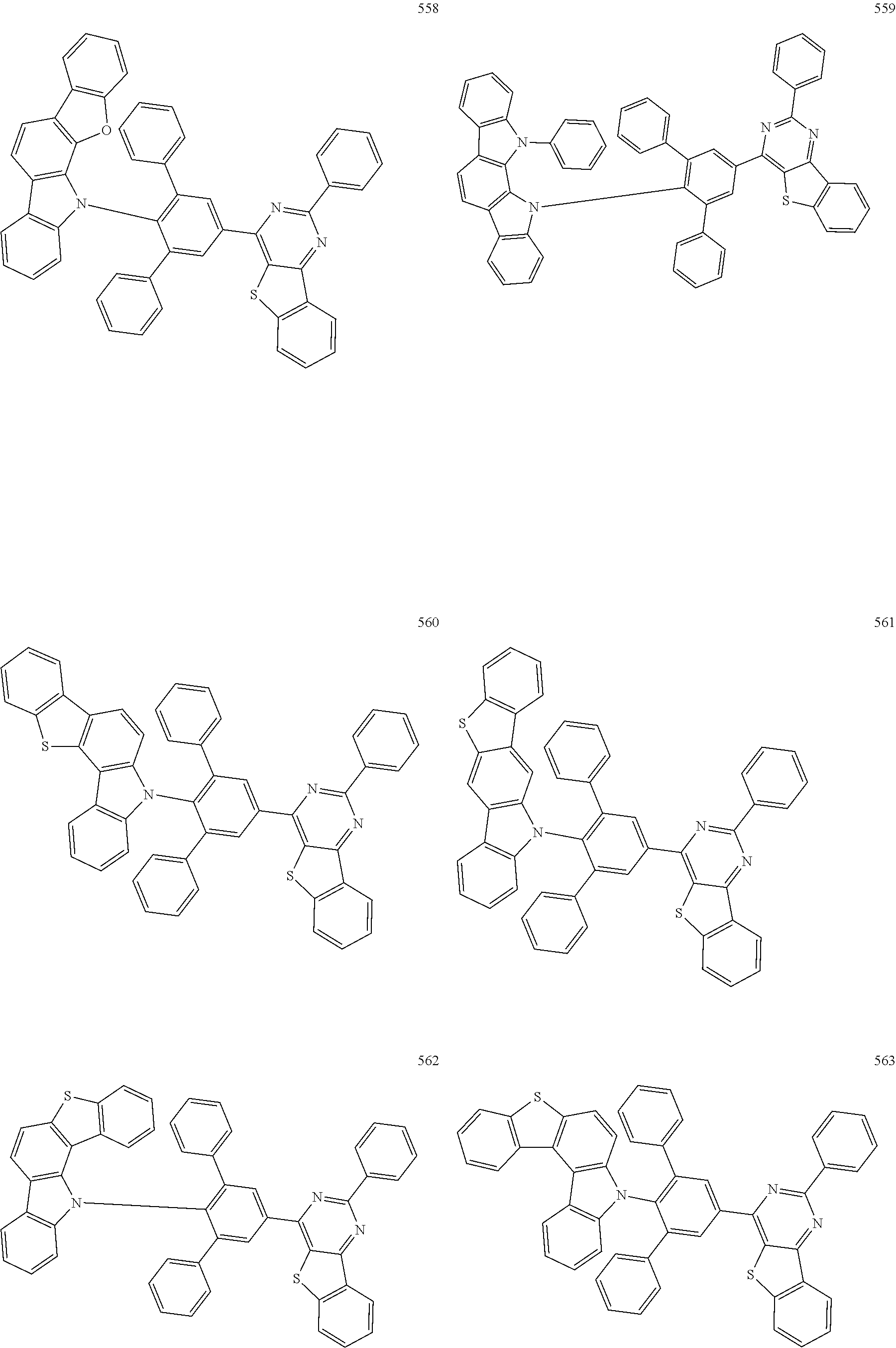

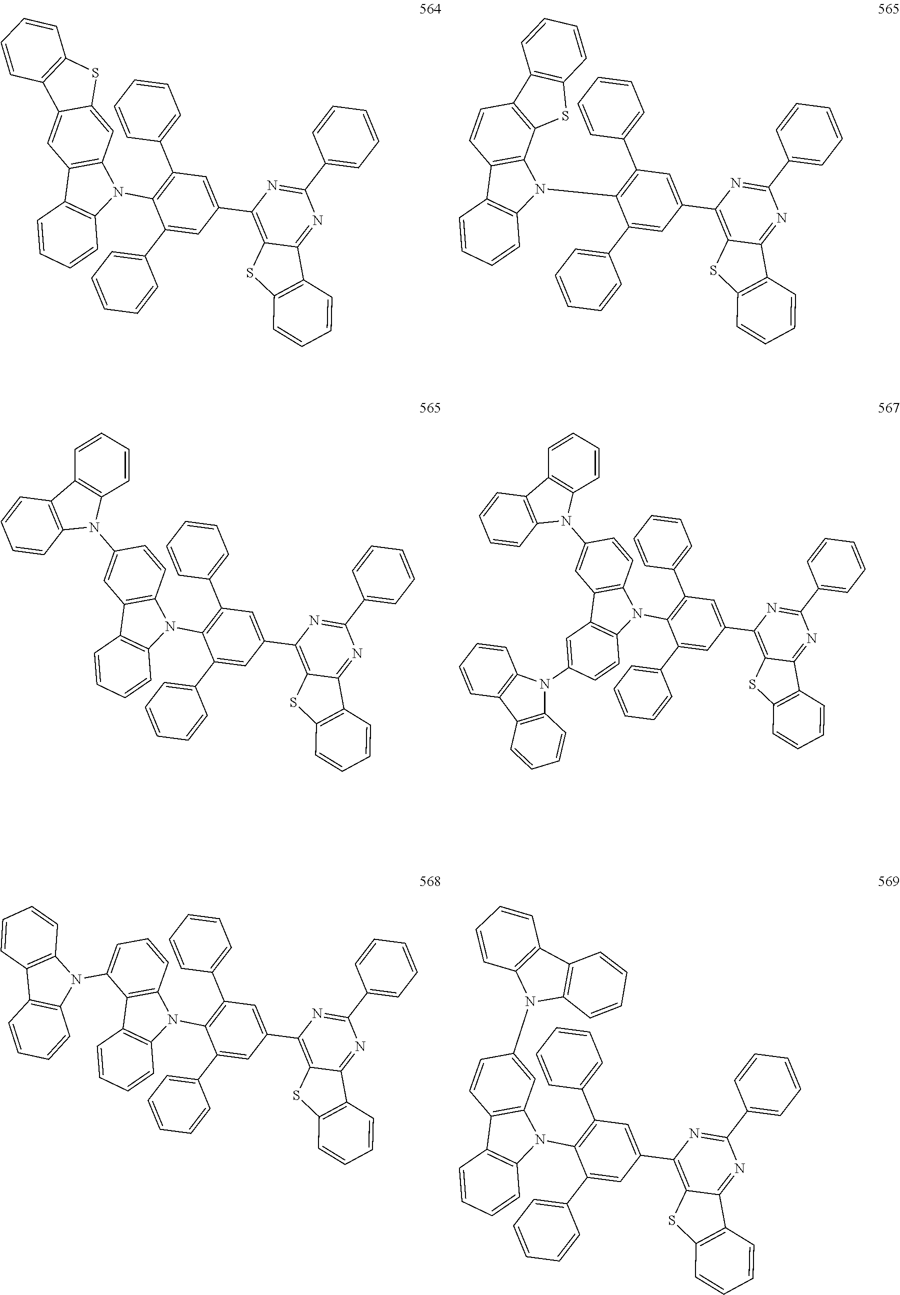

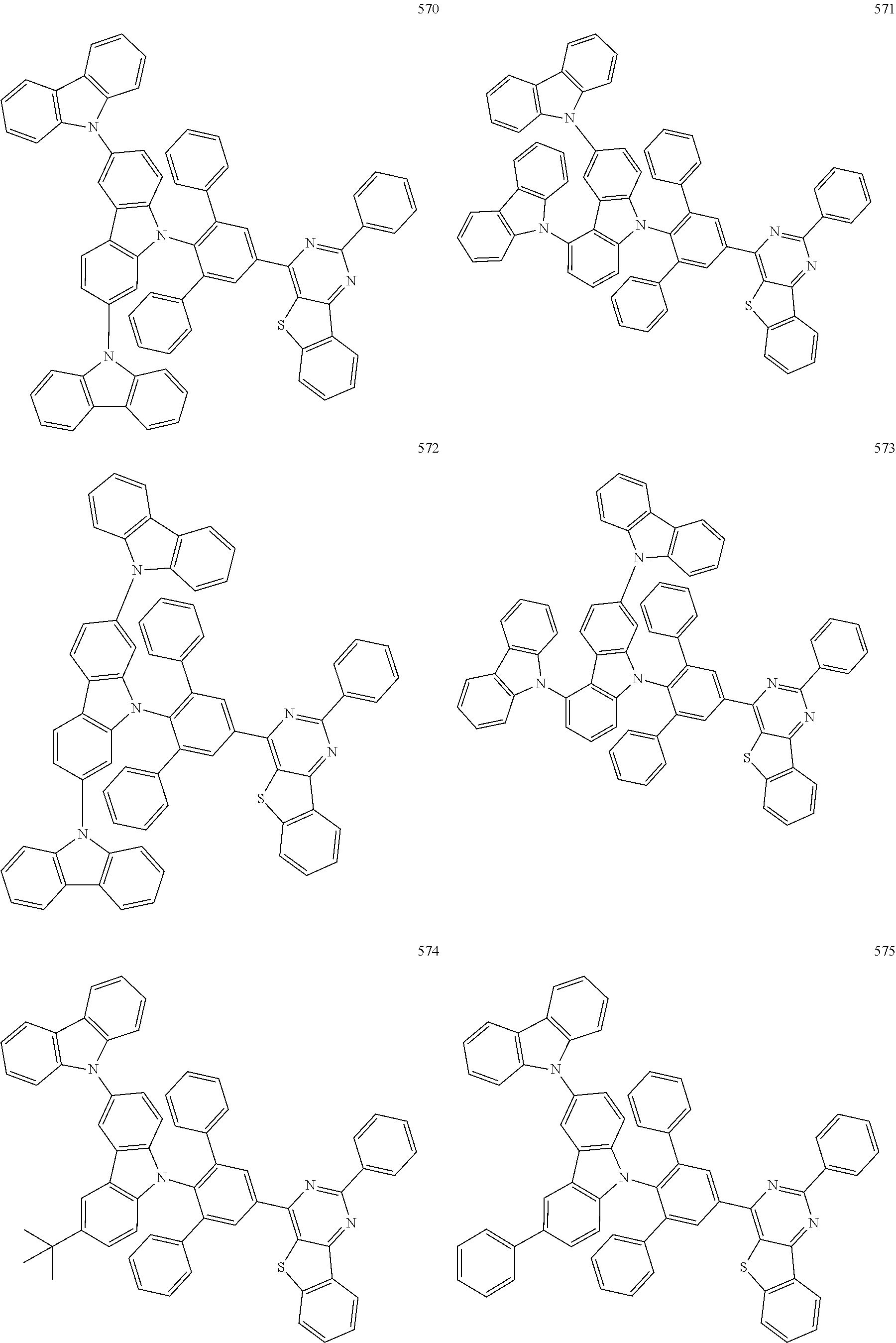

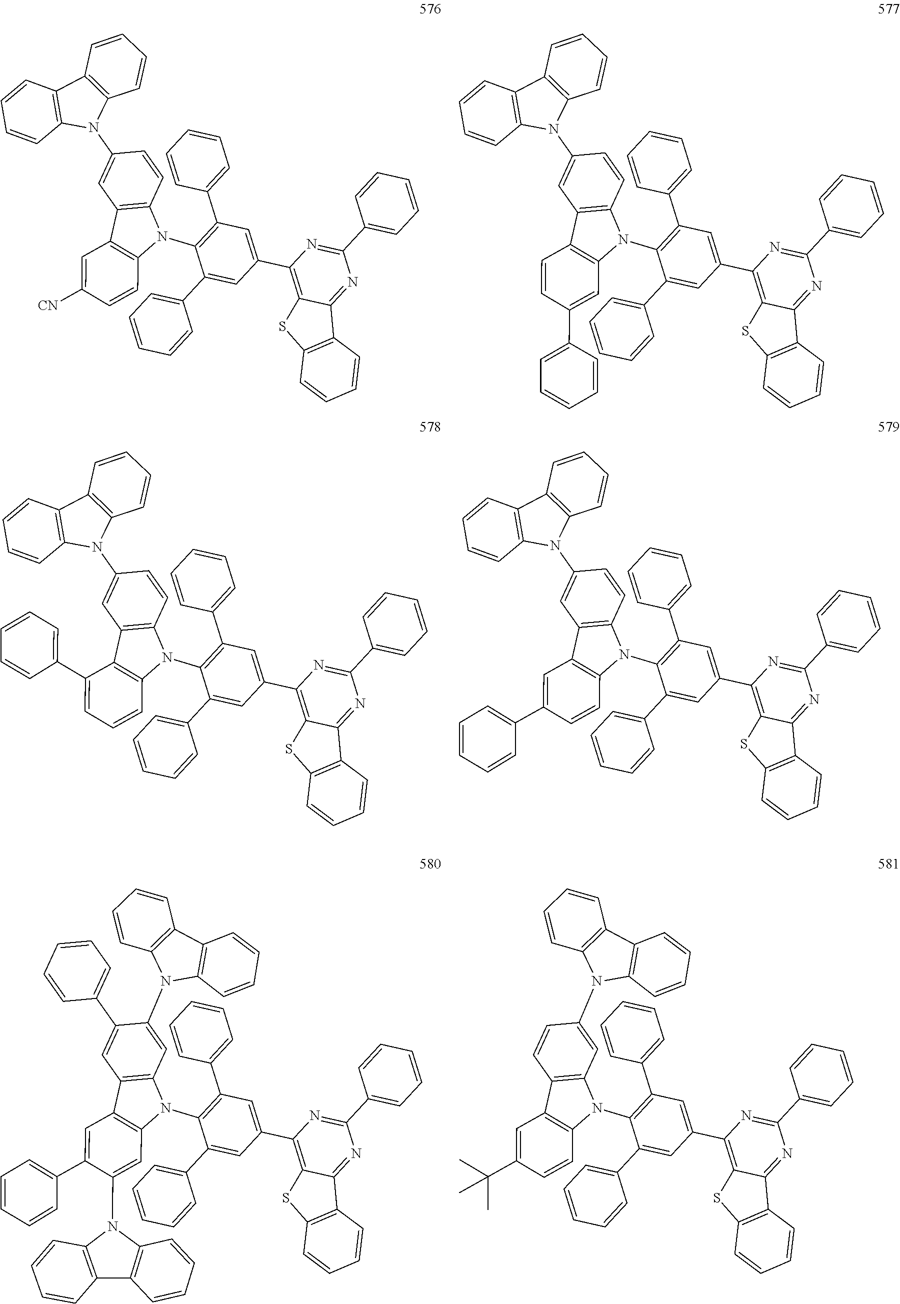

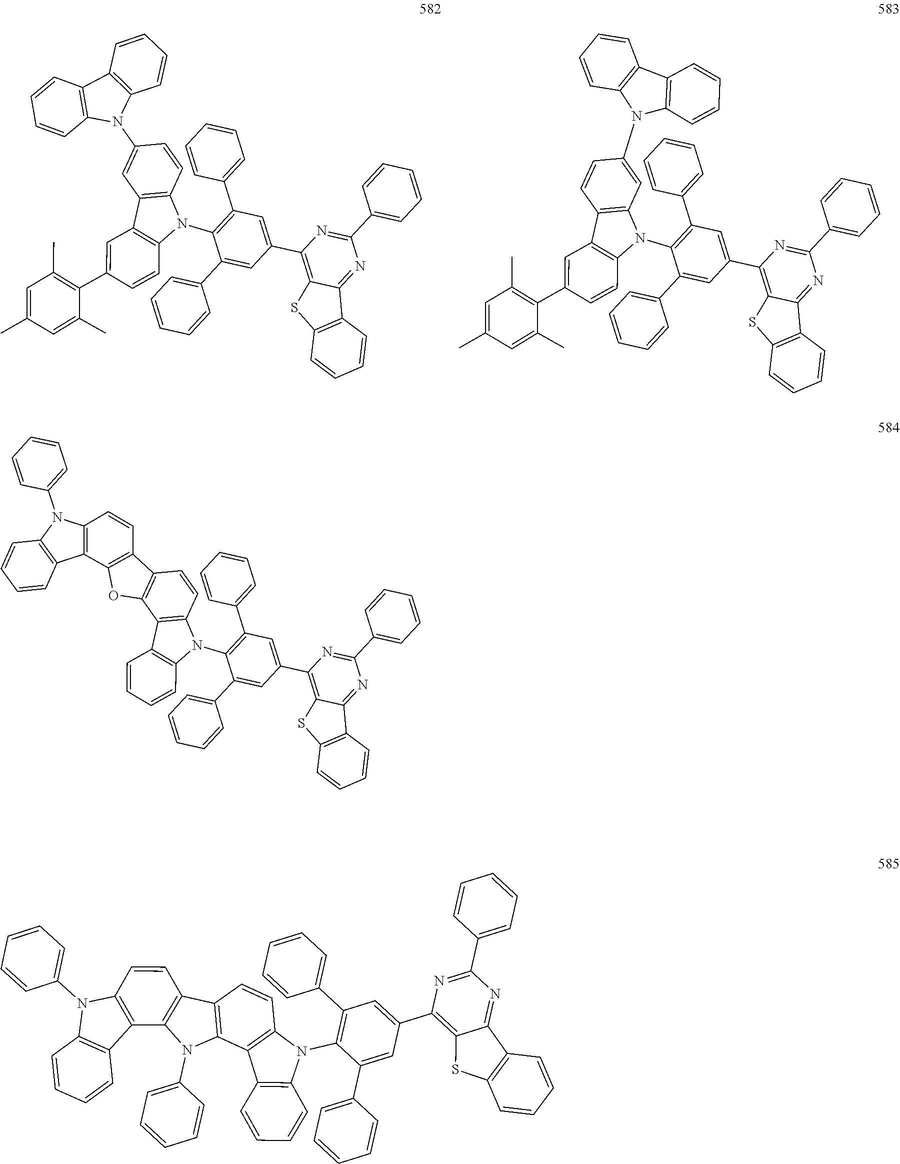

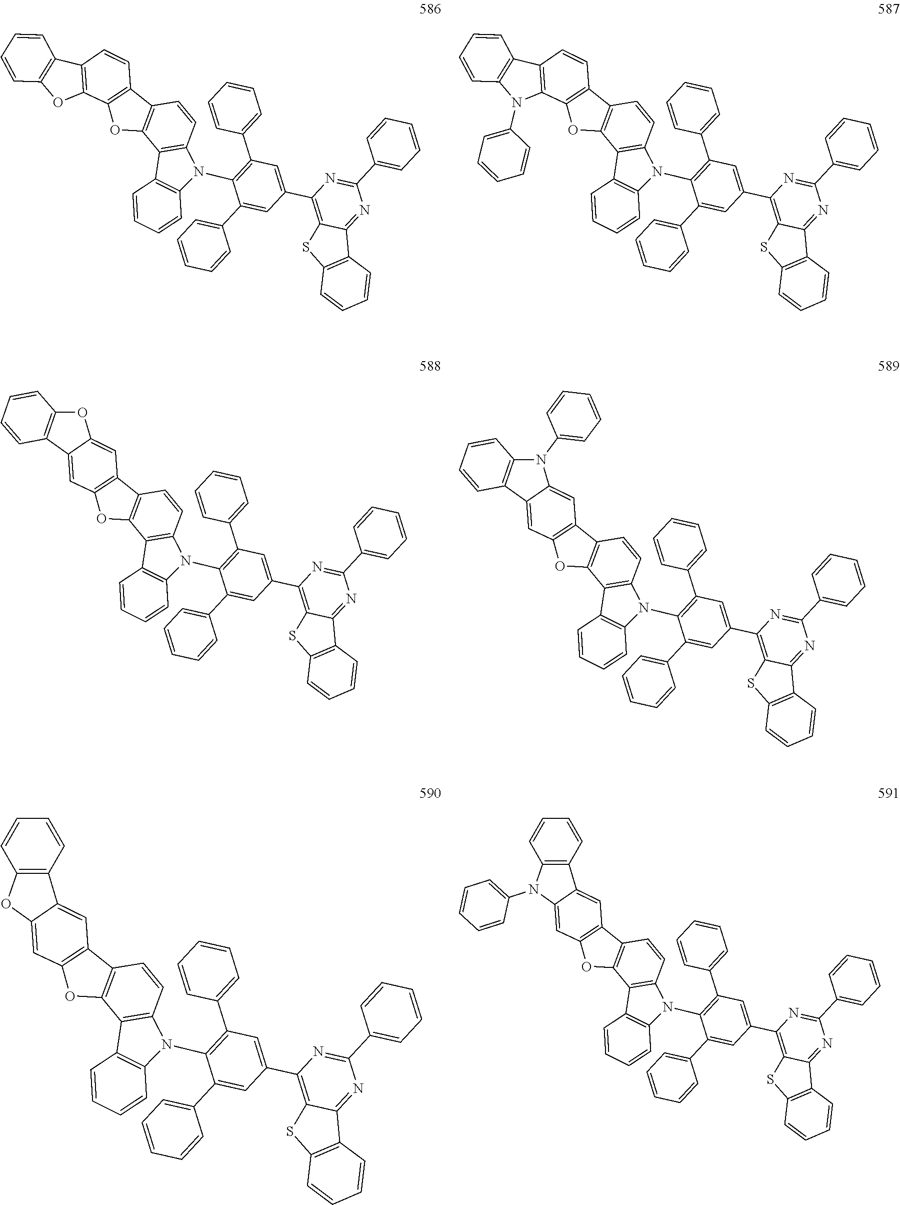

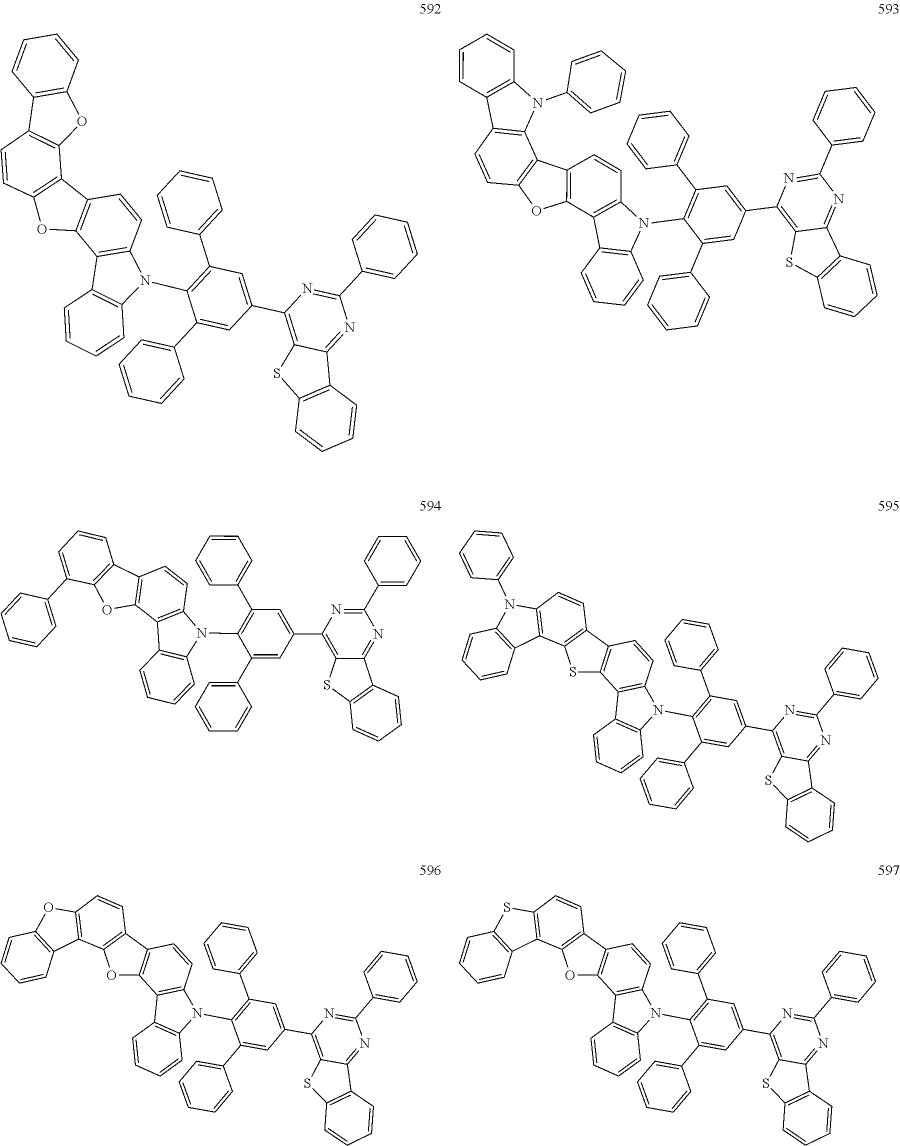

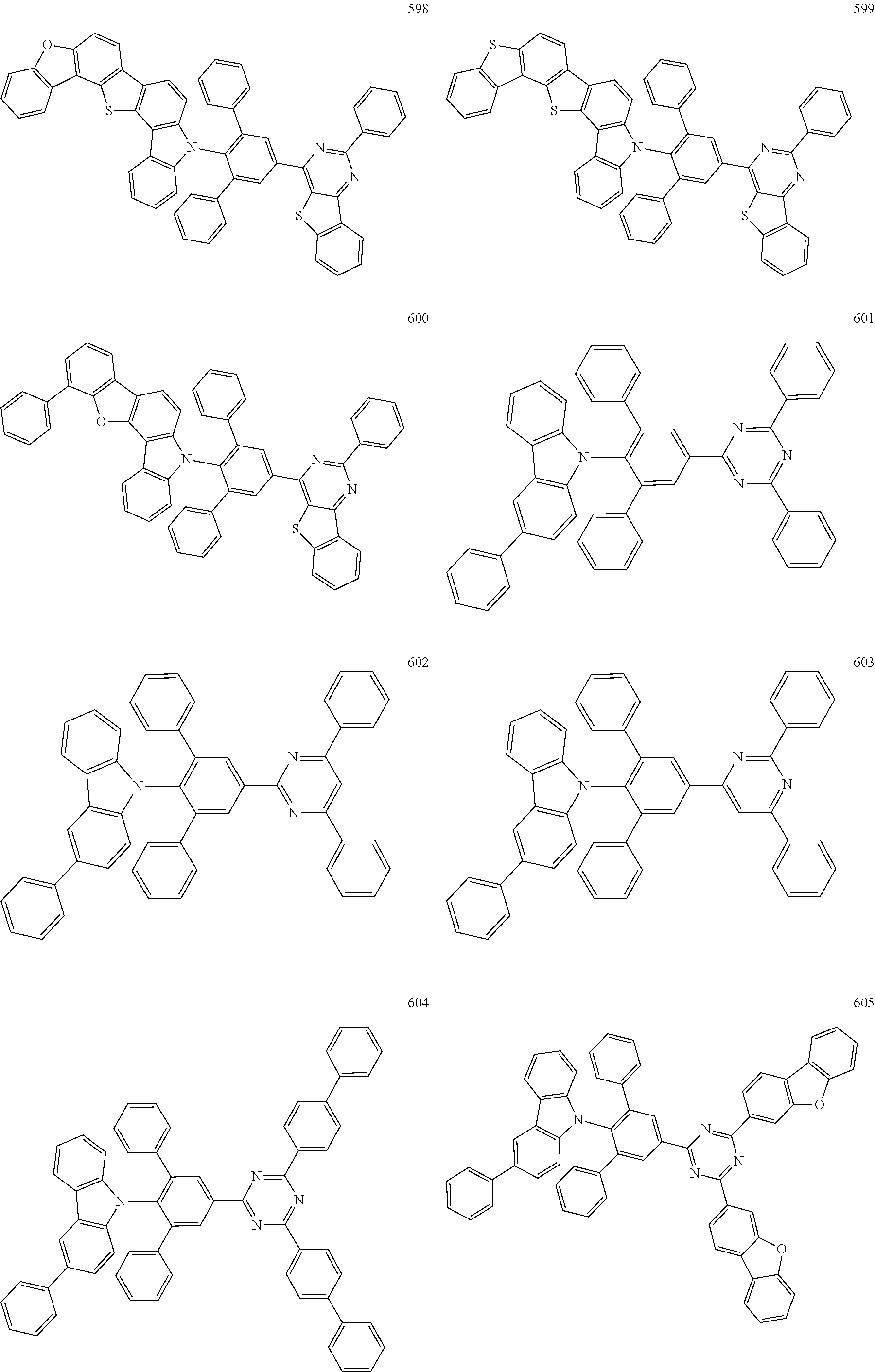

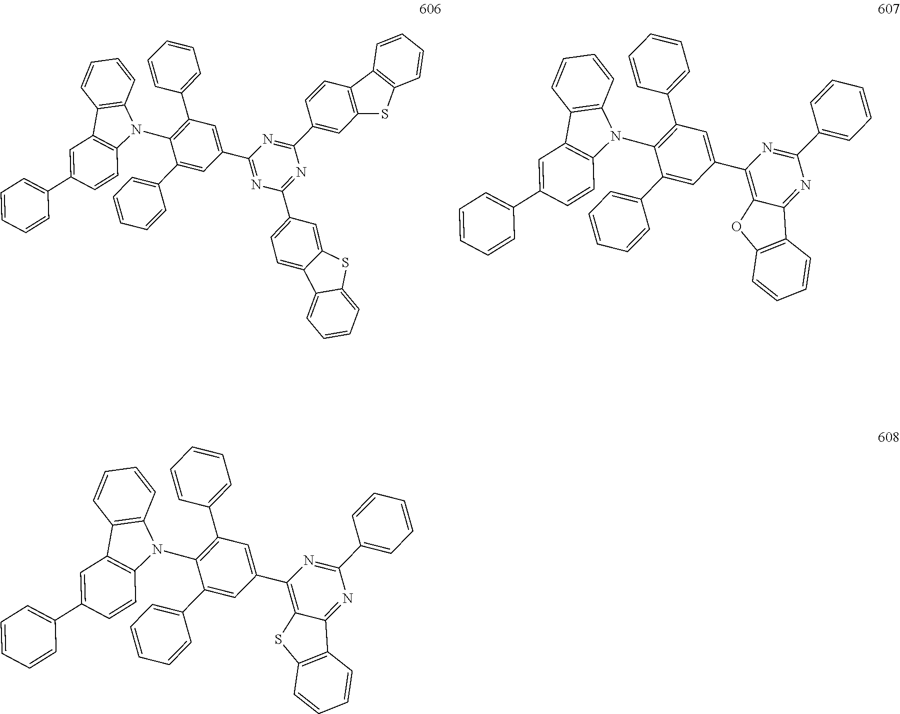 [SMILES: c1ccc(-c2ccc3c(c2)c2ccccc2n3-c2c(-c3ccccc3)cc(-c3nc(-c4ccc5c(c4)sc4ccccc45)nc(-c4ccc5c(c4)sc4ccccc45)n3)cc2-c2ccccc2)cc1 ; c1ccc(-c2ccc3c(c2)c2ccccc2n3-c2c(-c3ccccc3)cc(-c3nc(-c4ccccc4)nc4c3oc3ccccc34)cc2-c2ccccc2)cc1 ; c1ccc(-c2ccc3c(c2)c2ccccc2n3-c2c(-c3ccccc3)cc(-c3nc(-c4ccccc4)nc4c3sc3ccccc34)cc2-c2ccccc2)cc1]

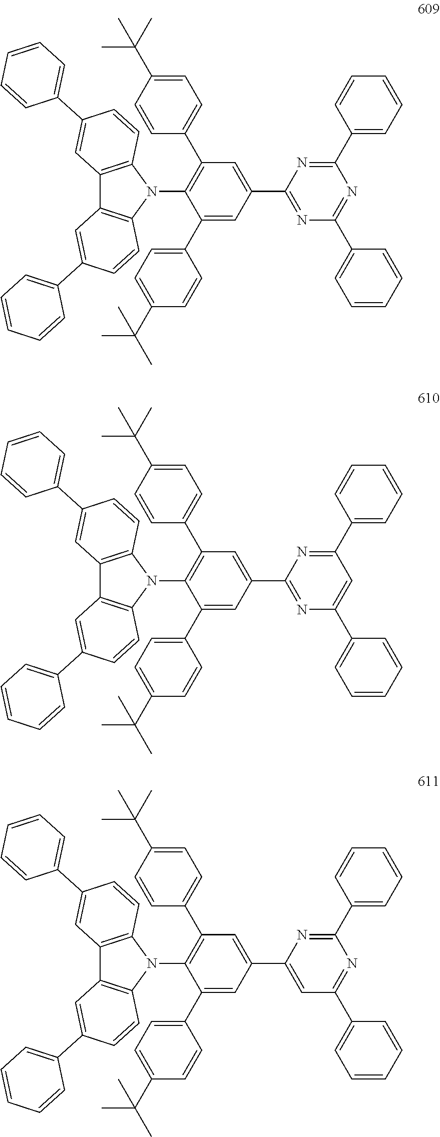

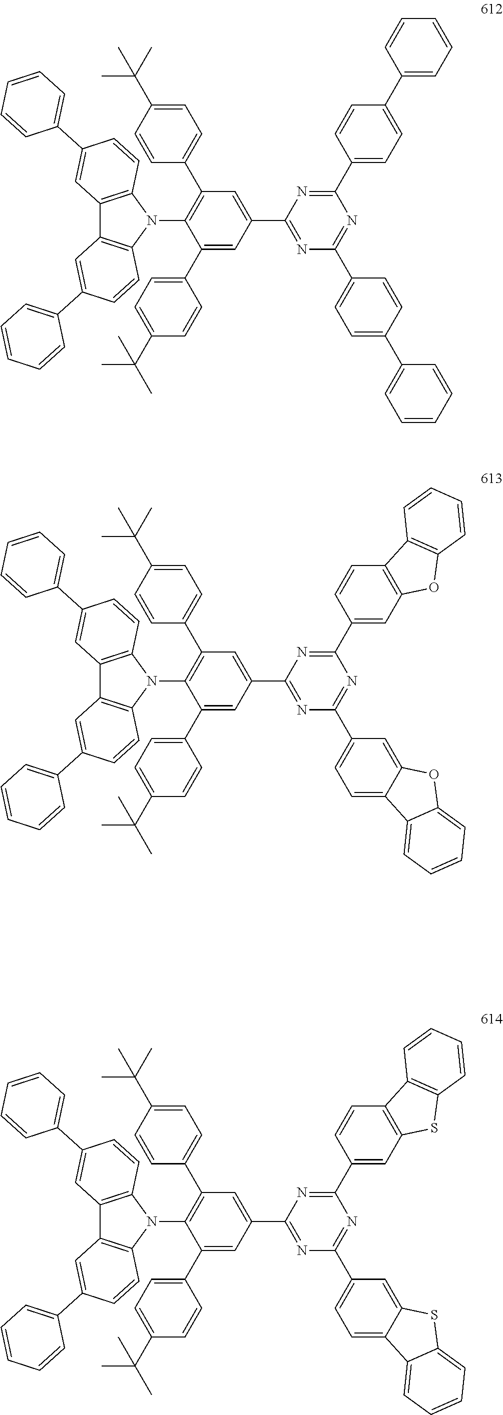 [SMILES: CC(C)(C)c1ccc(-c2cc(-c3nc(-c4ccc(-c5ccccc5)cc4)nc(-c4ccc(-c5ccccc5)cc4)n3)cc(-c3ccc(C(C)(C)C)cc3)c2-n2c3ccc(-c4ccccc4)cc3c3cc(-c4ccccc4)ccc32)cc1 ; CC(C)(C)c1ccc(-c2cc(-c3nc(-c4ccc5c(c4)oc4ccccc45)nc(-c4ccc5c(c4)oc4ccccc45)n3)cc(-c3ccc(C(C)(C)C)cc3)c2-n2c3ccc(-c4ccccc4)cc3c3cc(-c4ccccc4)ccc32)cc1 ; CC(C)(C)c1ccc(-c2cc(-c3nc(-c4ccc5c(c4)sc4ccccc45)nc(-c4ccc5c(c4)sc4ccccc45)n3)cc(-c3ccc(C(C)(C)C)cc3)c2-n2c3ccc(-c4ccccc4)cc3c3cc(-c4ccccc4)ccc32)cc1]

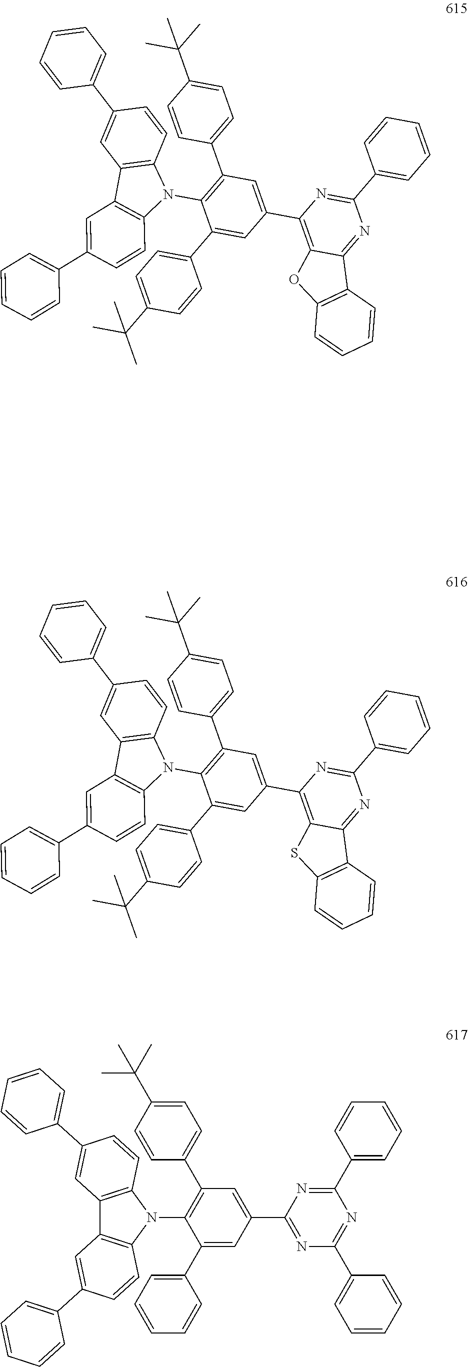 [SMILES: CC(C)(C)c1ccc(-c2cc(-c3nc(-c4ccccc4)nc4c3oc3ccccc34)cc(-c3ccc(C(C)(C)C)cc3)c2-n2c3ccc(-c4ccccc4)cc3c3cc(-c4ccccc4)ccc32)cc1 ; CC(C)(C)c1ccc(-c2cc(-c3nc(-c4ccccc4)nc4c3sc3ccccc34)cc(-c3ccc(C(C)(C)C)cc3)c2-n2c3ccc(-c4ccccc4)cc3c3cc(-c4ccccc4)ccc32)cc1 ; CC(C)(C)c1ccc(-c2cc(-c3nc(-c4ccccc4)nc(-c4ccccc4)n3)cc(-c3ccccc3)c2-n2c3ccc(-c4ccccc4)cc3c3cc(-c4ccccc4)ccc32)cc1]

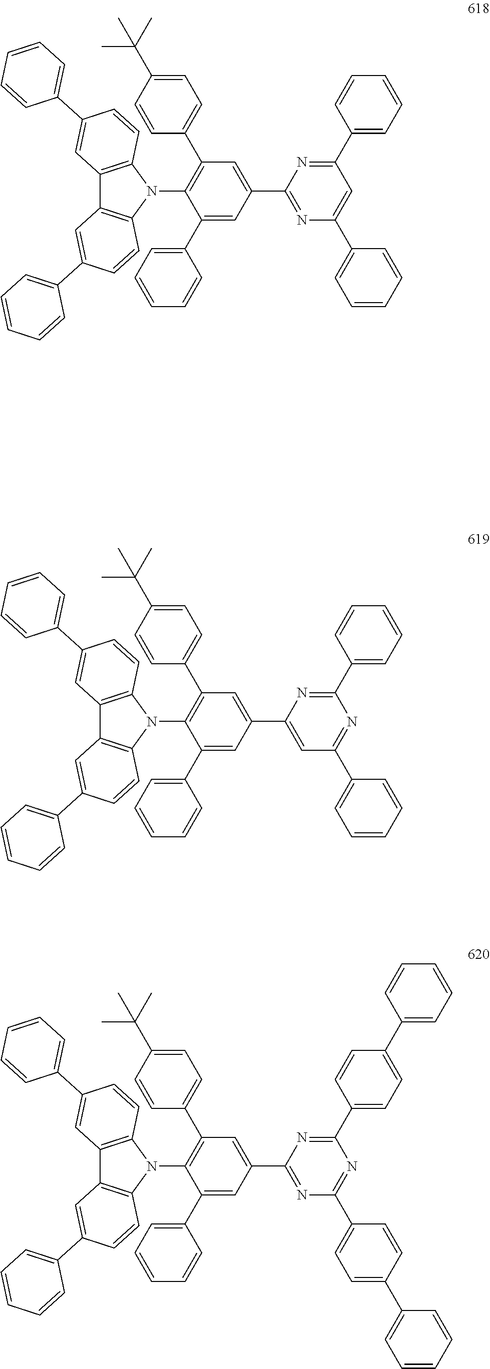 [SMILES: CC(C)(C)c1ccc(-c2cc(-c3nc(-c4ccccc4)cc(-c4ccccc4)n3)cc(-c3ccccc3)c2-n2c3ccc(-c4ccccc4)cc3c3cc(-c4ccccc4)ccc32)cc1 ; CC(C)(C)c1ccc(-c2cc(-c3cc(-c4ccccc4)nc(-c4ccccc4)n3)cc(-c3ccccc3)c2-n2c3ccc(-c4ccccc4)cc3c3cc(-c4ccccc4)ccc32)cc1 ; CC(C)(C)c1ccc(-c2cc(-c3nc(-c4ccc(-c5ccccc5)cc4)nc(-c4ccc(-c5ccccc5)cc4)n3)cc(-c3ccccc3)c2-n2c3ccc(-c4ccccc4)cc3c3cc(-c4ccccc4)ccc32)cc1]

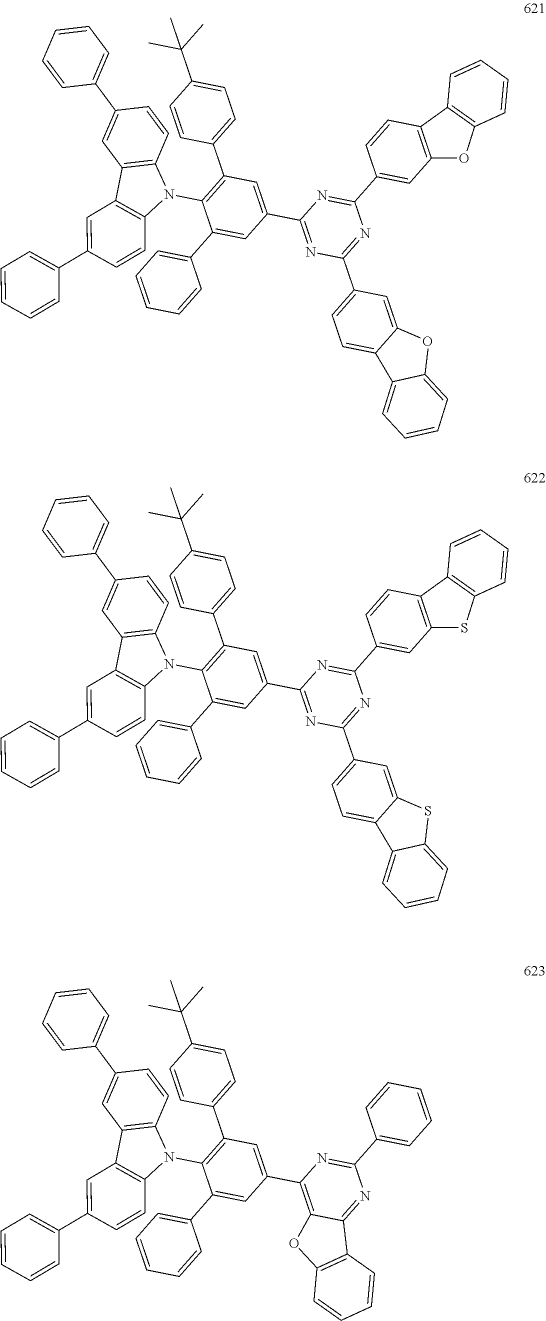 [SMILES: CC(C)(C)c1ccc(-c2cc(-c3nc(-c4ccc5c(c4)oc4ccccc45)nc(-c4ccc5c(c4)oc4ccccc45)n3)cc(-c3ccccc3)c2-n2c3ccc(-c4ccccc4)cc3c3cc(-c4ccccc4)ccc32)cc1 ; CC(C)(C)c1ccc(-c2cc(-c3nc(-c4ccc5c(c4)sc4ccccc45)nc(-c4ccc5c(c4)sc4ccccc45)n3)cc(-c3ccccc3)c2-n2c3ccc(-c4ccccc4)cc3c3cc(-c4ccccc4)ccc32)cc1 ; CC(C)(C)c1ccc(-c2cc(-c3nc(-c4ccccc4)nc4c3oc3ccccc34)cc(-c3ccccc3)c2-n2c3ccc(-c4ccccc4)cc3c3cc(-c4ccccc4)ccc32)cc1]

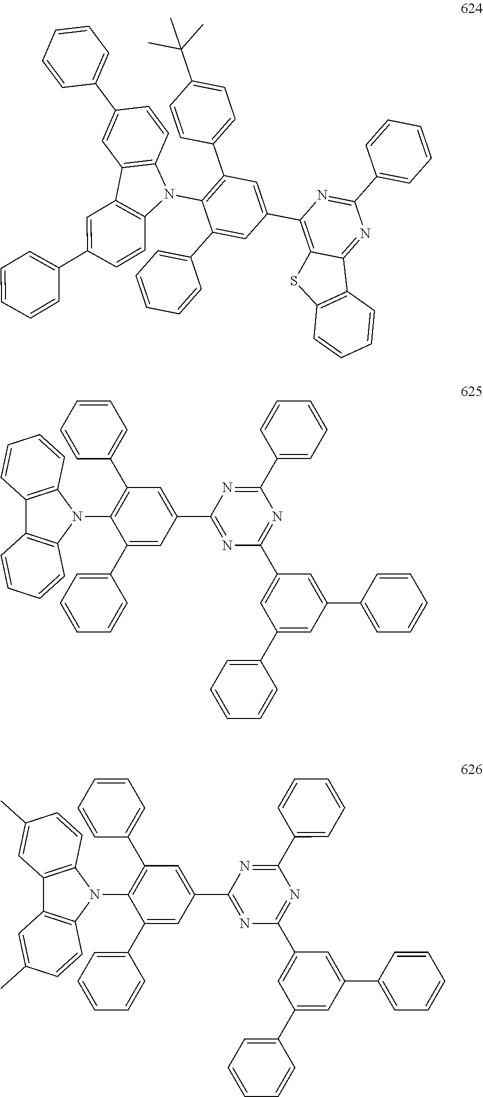 [SMILES: CC(C)(C)c1ccc(-c2cc(-c3nc(-c4ccccc4)nc4c3sc3ccccc34)cc(-c3ccccc3)c2-n2c3ccc(-c4ccccc4)cc3c3cc(-c4ccccc4)ccc32)cc1 ; c1ccc(-c2cc(-c3ccccc3)cc(-c3nc(-c4ccccc4)nc(-c4cc(-c5ccccc5)c(-n5c6ccccc6c6ccccc65)c(-c5ccccc5)c4)n3)c2)cc1 ; Cc1ccc2c(c1)c1cc(C)ccc1n2-c1c(-c2ccccc2)cc(-c2nc(-c3ccccc3)nc(-c3cc(-c4ccccc4)cc(-c4ccccc4)c3)n2)cc1-c1ccccc1]

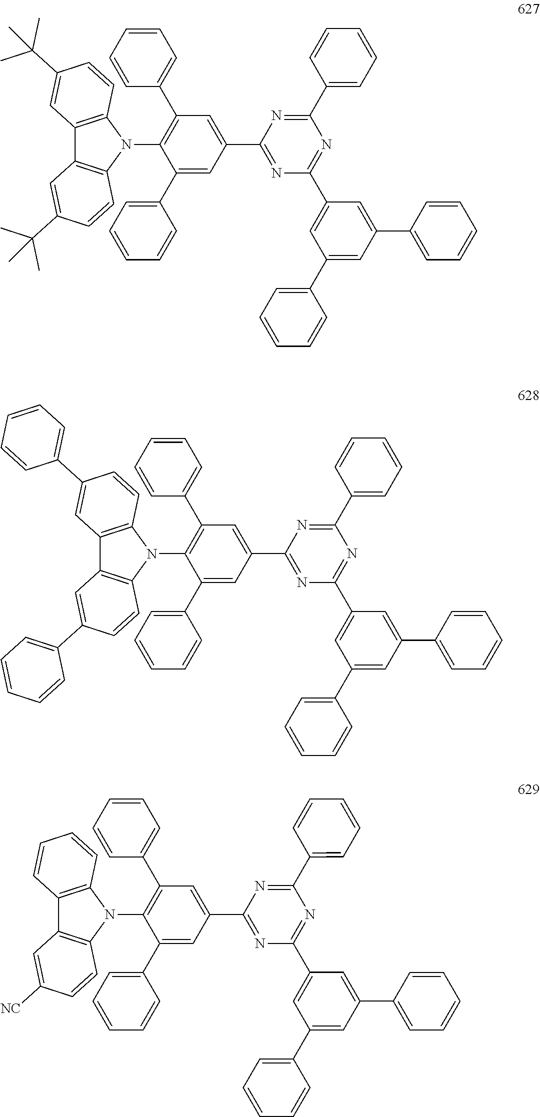

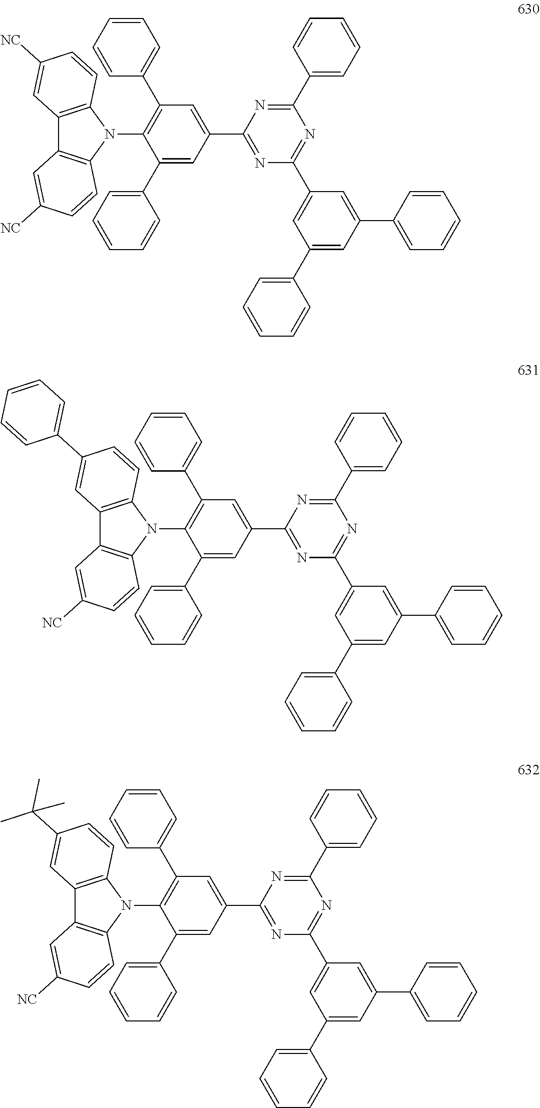 [SMILES: N#Cc1ccc2c(c1)c1cc(C#N)ccc1n2-c1c(-c2ccccc2)cc(-c2nc(-c3ccccc3)nc(-c3cc(-c4ccccc4)cc(-c4ccccc4)c3)n2)cc1-c1ccccc1 ; N#Cc1ccc2c(c1)c1cc(-c3ccccc3)ccc1n2-c1c(-c2ccccc2)cc(-c2nc(-c3ccccc3)nc(-c3cc(-c4ccccc4)cc(-c4ccccc4)c3)n2)cc1-c1ccccc1 ; CC(C)(C)c1ccc2c(c1)c1cc(C#N)ccc1n2-c1c(-c2ccccc2)cc(-c2nc(-c3ccccc3)nc(-c3cc(-c4ccccc4)cc(-c4ccccc4)c3)n2)cc1-c1ccccc1]

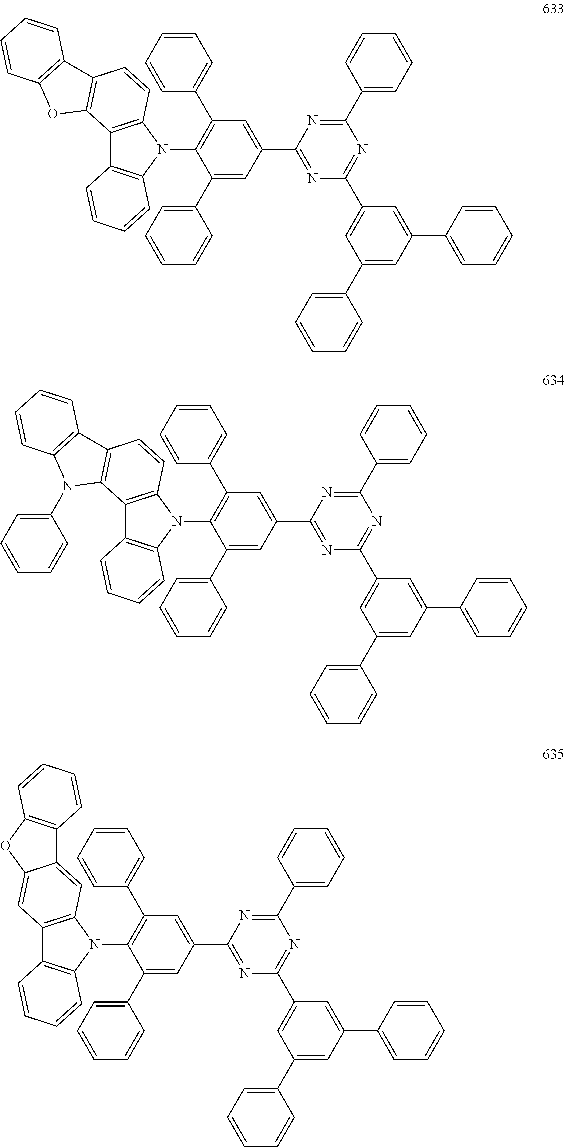 [SMILES: c1ccc(-c2cc(-c3ccccc3)cc(-c3nc(-c4ccccc4)nc(-c4cc(-c5ccccc5)c(-n5c6ccccc6c6c7oc8ccccc8c7ccc65)c(-c5ccccc5)c4)n3)c2)cc1 ; c1ccc(-c2cc(-c3ccccc3)cc(-c3nc(-c4ccccc4)nc(-c4cc(-c5ccccc5)c(-n5c6ccccc6c6c5ccc5c7ccccc7n(-c7ccccc7)c56)c(-c5ccccc5)c4)n3)c2)cc1 ; c1ccc(-c2cc(-c3ccccc3)cc(-c3nc(-c4ccccc4)nc(-c4cc(-c5ccccc5)c(-n5c6ccccc6c6cc7oc8ccccc8c7cc65)c(-c5ccccc5)c4)n3)c2)cc1]

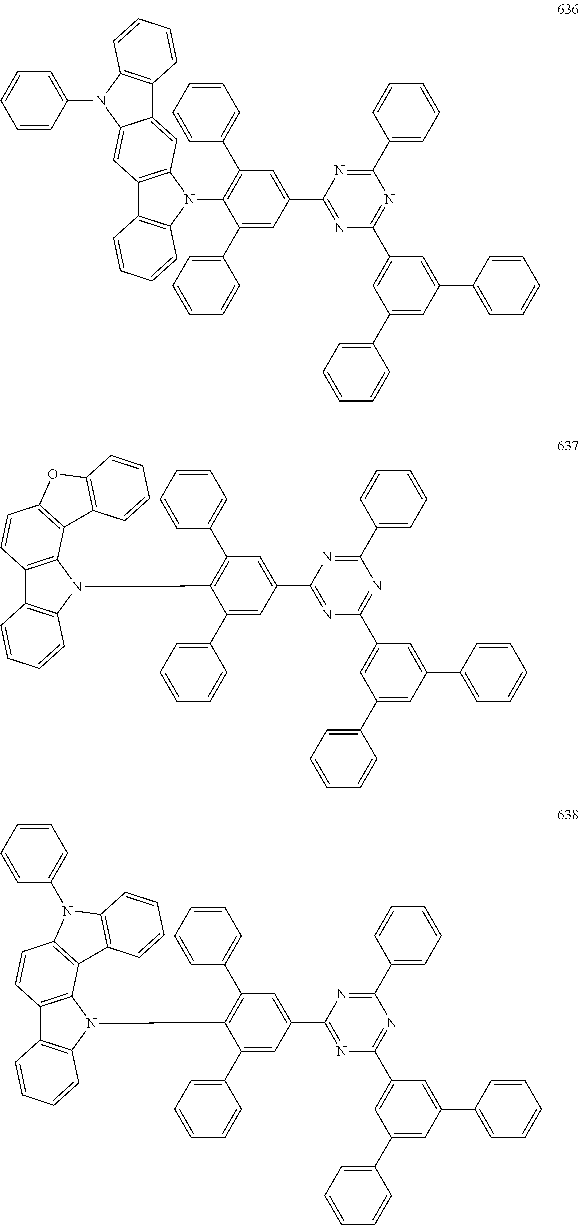 [SMILES: c1ccc(-c2cc(-c3ccccc3)cc(-c3nc(-c4ccccc4)nc(-c4cc(-c5ccccc5)c(-n5c6ccccc6c6cc7c(cc65)c5ccccc5n7-c5ccccc5)c(-c5ccccc5)c4)n3)c2)cc1 ; c1ccc(-c2cc(-c3ccccc3)cc(-c3nc(-c4ccccc4)nc(-c4cc(-c5ccccc5)c(-n5c6ccccc6c6ccc7oc8ccccc8c7c65)c(-c5ccccc5)c4)n3)c2)cc1 ; c1ccc(-c2cc(-c3ccccc3)cc(-c3nc(-c4ccccc4)nc(-c4cc(-c5ccccc5)c(-n5c6ccccc6c6ccc7c(c8ccccc8n7-c7ccccc7)c65)c(-c5ccccc5)c4)n3)c2)cc1]

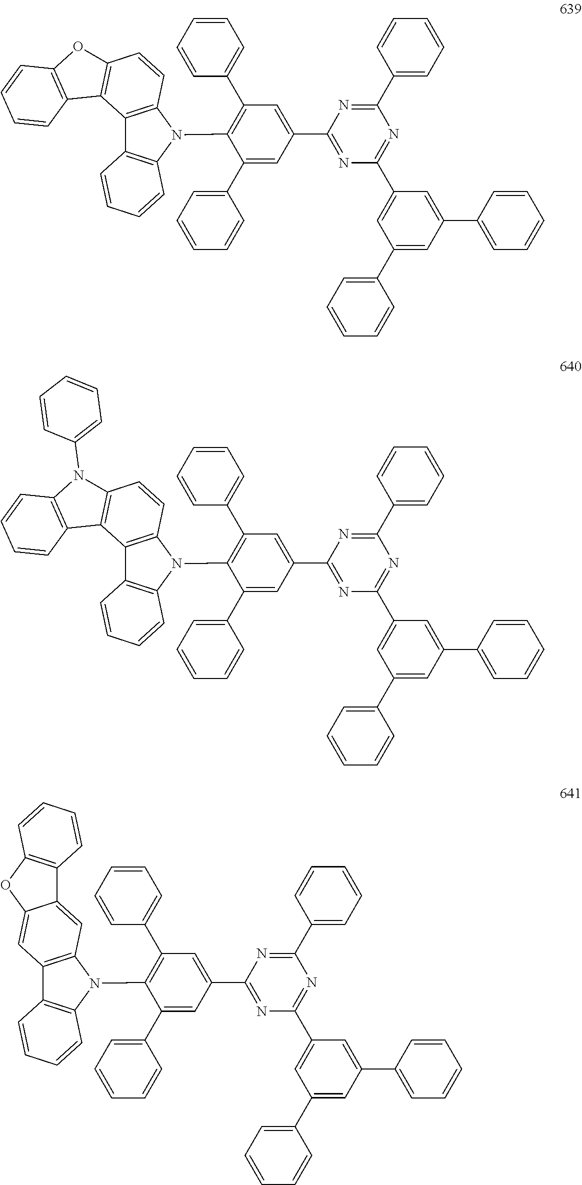 [SMILES: c1ccc(-c2cc(-c3ccccc3)cc(-c3nc(-c4ccccc4)nc(-c4cc(-c5ccccc5)c(-n5c6ccccc6c6c7c(ccc65)oc5ccccc57)c(-c5ccccc5)c4)n3)c2)cc1 ; c1ccc(-c2cc(-c3ccccc3)cc(-c3nc(-c4ccccc4)nc(-c4cc(-c5ccccc5)c(-n5c6ccccc6c6c7c8ccccc8n(-c8ccccc8)c7ccc65)c(-c5ccccc5)c4)n3)c2)cc1 ; c1ccc(-c2cc(-c3ccccc3)cc(-c3nc(-c4ccccc4)nc(-c4cc(-c5ccccc5)c(-n5c6ccccc6c6cc7oc8ccccc8c7cc65)c(-c5ccccc5)c4)n3)c2)cc1]

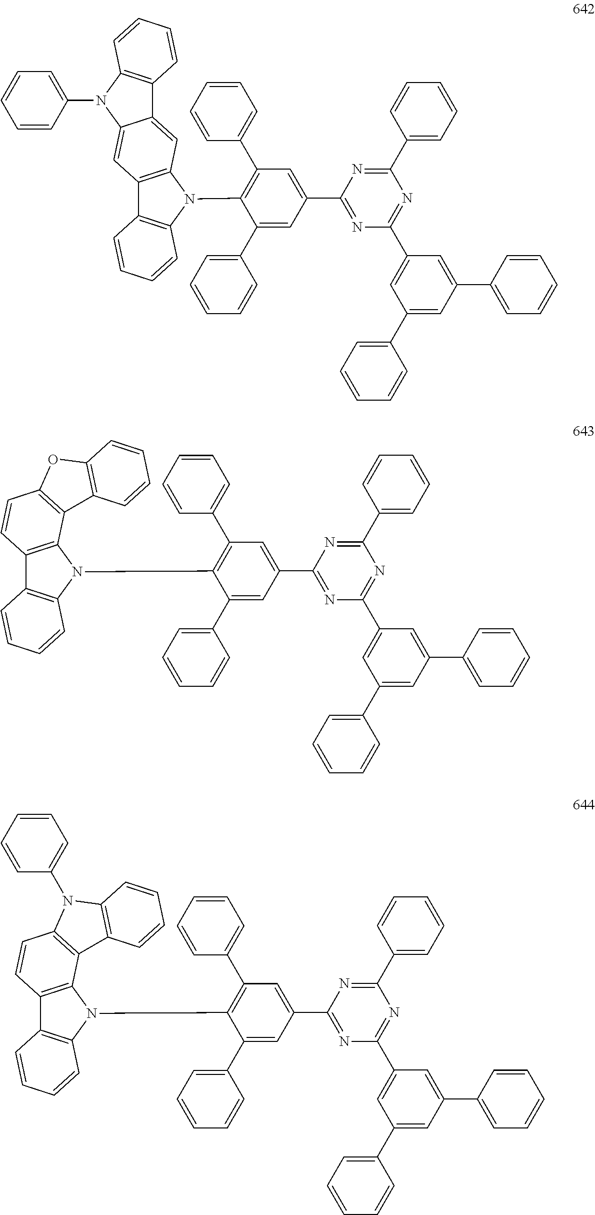 [SMILES: c1ccc(-c2cc(-c3ccccc3)cc(-c3nc(-c4ccccc4)nc(-c4cc(-c5ccccc5)c(-n5c6ccccc6c6cc7c(cc65)c5ccccc5n7-c5ccccc5)c(-c5ccccc5)c4)n3)c2)cc1 ; c1ccc(-c2cc(-c3ccccc3)cc(-c3nc(-c4ccccc4)nc(-c4cc(-c5ccccc5)c(-n5c6ccccc6c6ccc7oc8ccccc8c7c65)c(-c5ccccc5)c4)n3)c2)cc1 ; c1ccc(-c2cc(-c3ccccc3)cc(-c3nc(-c4ccccc4)nc(-c4cc(-c5ccccc5)c(-n5c6ccccc6c6ccc7c(c8ccccc8n7-c7ccccc7)c65)c(-c5ccccc5)c4)n3)c2)cc1]

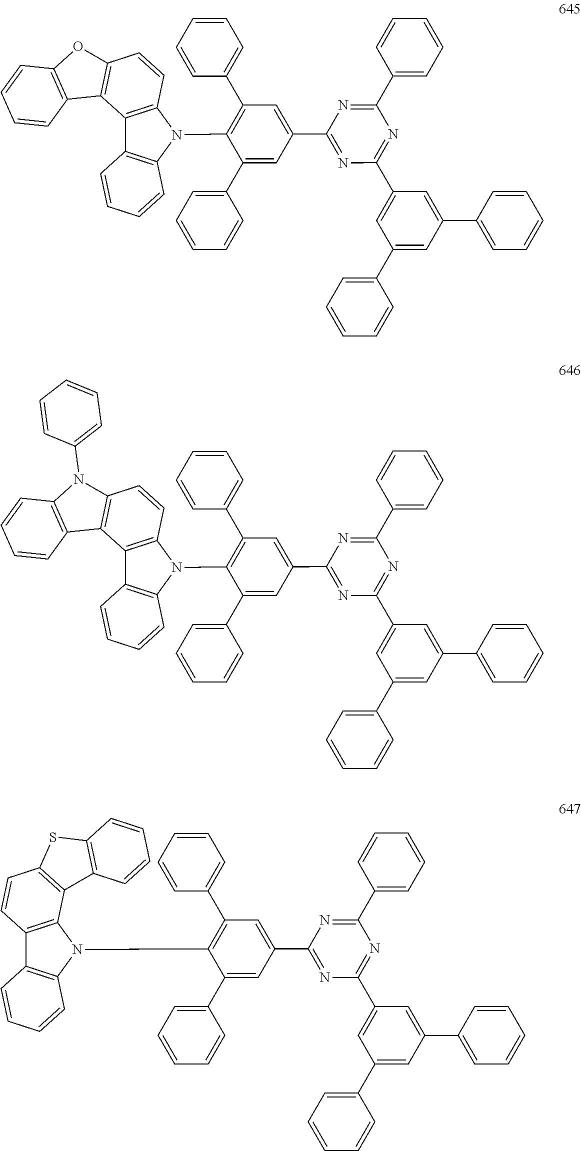 [SMILES: c1ccc(-c2cc(-c3ccccc3)cc(-c3nc(-c4ccccc4)nc(-c4cc(-c5ccccc5)c(-n5c6ccccc6c6c7c(ccc65)oc5ccccc57)c(-c5ccccc5)c4)n3)c2)cc1 ; c1ccc(-c2cc(-c3ccccc3)cc(-c3nc(-c4ccccc4)nc(-c4cc(-c5ccccc5)c(-n5c6ccccc6c6c7c8ccccc8n(-c8ccccc8)c7ccc65)c(-c5ccccc5)c4)n3)c2)cc1 ; c1ccc(-c2cc(-c3ccccc3)cc(-c3nc(-c4ccccc4)nc(-c4cc(-c5ccccc5)c(-n5c6ccccc6c6ccc7sc8ccccc8c7c65)c(-c5ccccc5)c4)n3)c2)cc1]

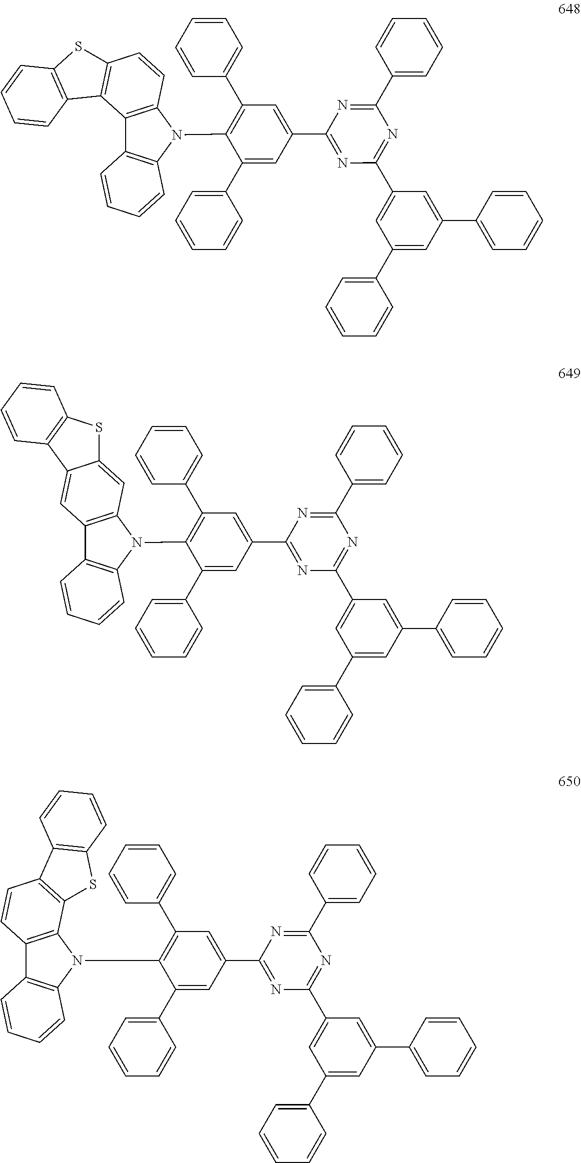 [SMILES: c1ccc(-c2cc(-c3ccccc3)cc(-c3nc(-c4ccccc4)nc(-c4cc(-c5ccccc5)c(-n5c6ccccc6c6c7c(ccc65)sc5ccccc57)c(-c5ccccc5)c4)n3)c2)cc1 ; c1ccc(-c2cc(-c3ccccc3)cc(-c3nc(-c4ccccc4)nc(-c4cc(-c5ccccc5)c(-n5c6ccccc6c6cc7c(cc65)sc5ccccc57)c(-c5ccccc5)c4)n3)c2)cc1 ; c1ccc(-c2cc(-c3ccccc3)cc(-c3nc(-c4ccccc4)nc(-c4cc(-c5ccccc5)c(-n5c6ccccc6c6ccc7c8ccccc8sc7c65)c(-c5ccccc5)c4)n3)c2)cc1]

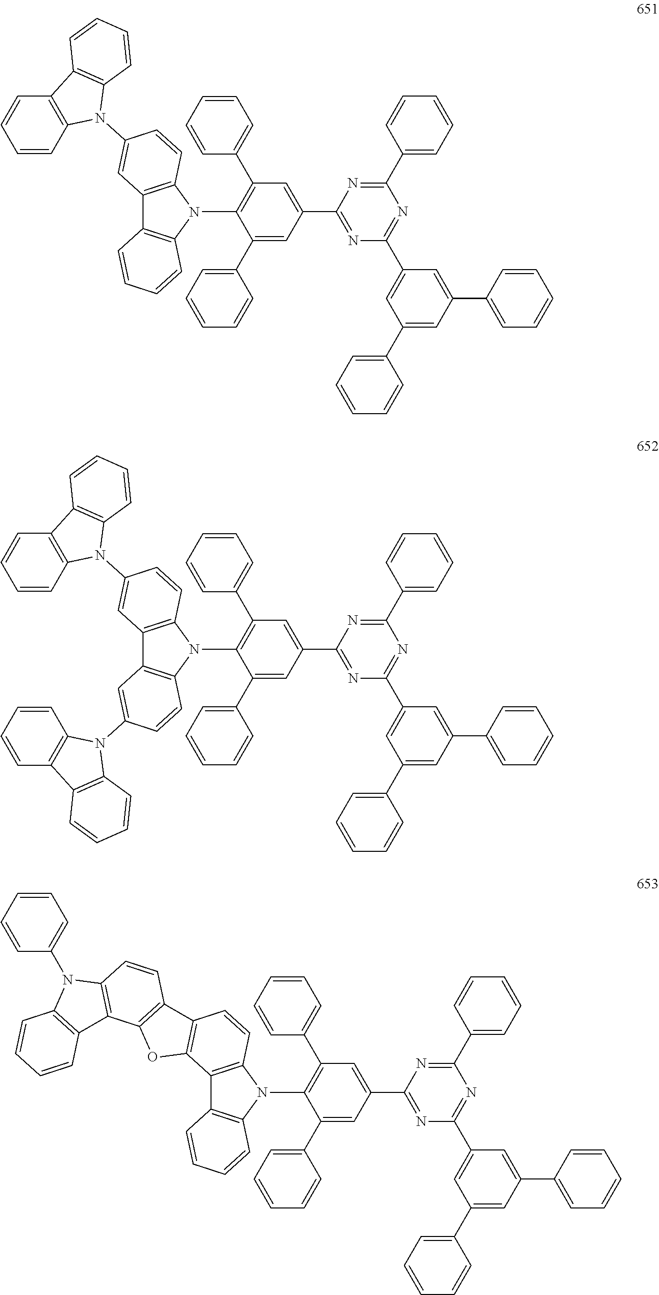 [SMILES: c1ccc(-c2cc(-c3ccccc3)cc(-c3nc(-c4ccccc4)nc(-c4cc(-c5ccccc5)c(-n5c6ccccc6c6cc(-n7c8ccccc8c8ccccc87)ccc65)c(-c5ccccc5)c4)n3)c2)cc1 ; c1ccc(-c2cc(-c3ccccc3)cc(-c3nc(-c4ccccc4)nc(-c4cc(-c5ccccc5)c(-n5c6ccc(-n7c8ccccc8c8ccccc87)cc6c6cc(-n7c8ccccc8c8ccccc87)ccc65)c(-c5ccccc5)c4)n3)c2)cc1 ; c1ccc(-c2cc(-c3ccccc3)cc(-c3nc(-c4ccccc4)nc(-c4cc(-c5ccccc5)c(-n5c6ccccc6c6c7oc8c(ccc9c8c8ccccc8n9-c8ccccc8)c7ccc65)c(-c5ccccc5)c4)n3)c2)cc1]

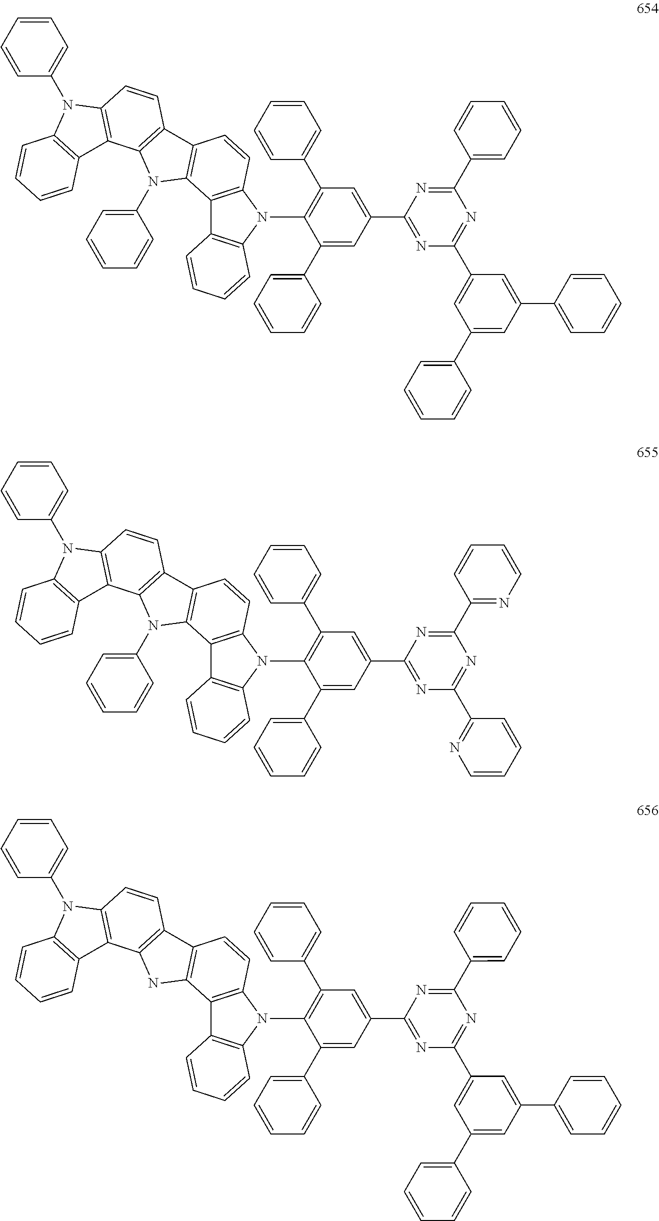 [SMILES: c1ccc(-c2cc(-c3ccccc3)cc(-c3nc(-c4ccccc4)nc(-c4cc(-c5ccccc5)c(-n5c6ccccc6c6c5ccc5c7ccc8c(c9ccccc9n8-c8ccccc8)c7n(-c7ccccc7)c56)c(-c5ccccc5)c4)n3)c2)cc1 ; c1ccc(-c2cc(-c3nc(-c4ccccn4)nc(-c4ccccn4)n3)cc(-c3ccccc3)c2-n2c3ccccc3c3c2ccc2c4ccc5c(c6ccccc6n5-c5ccccc5)c4n(-c4ccccc4)c23)cc1 ; c1ccc(-c2cc(-c3ccccc3)cc(-c3nc(-c4ccccc4)nc(-c4cc(-c5ccccc5)c(-n5c6ccccc6c6c7[nH]c8c(ccc9c8c8ccccc8n9-c8ccccc8)c7ccc65)c(-c5ccccc5)c4)n3)c2)cc1]

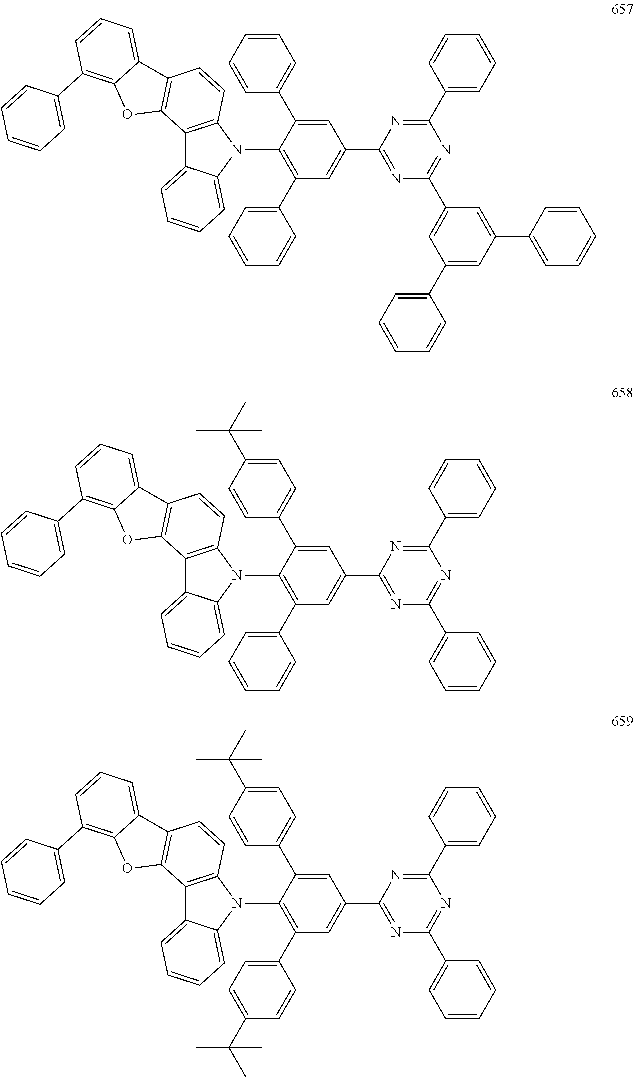

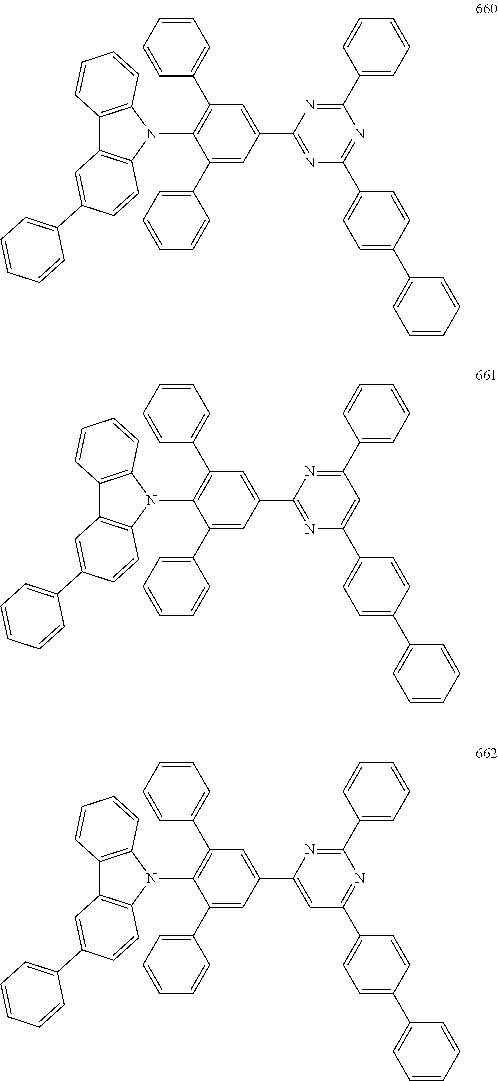 [SMILES: c1ccc(-c2ccc(-c3nc(-c4ccccc4)nc(-c4cc(-c5ccccc5)c(-n5c6ccccc6c6cc(-c7ccccc7)ccc65)c(-c5ccccc5)c4)n3)cc2)cc1 ; c1ccc(-c2ccc(-c3cc(-c4ccccc4)nc(-c4cc(-c5ccccc5)c(-n5c6ccccc6c6cc(-c7ccccc7)ccc65)c(-c5ccccc5)c4)n3)cc2)cc1 ; c1ccc(-c2ccc(-c3cc(-c4cc(-c5ccccc5)c(-n5c6ccccc6c6cc(-c7ccccc7)ccc65)c(-c5ccccc5)c4)nc(-c4ccccc4)n3)cc2)cc1]

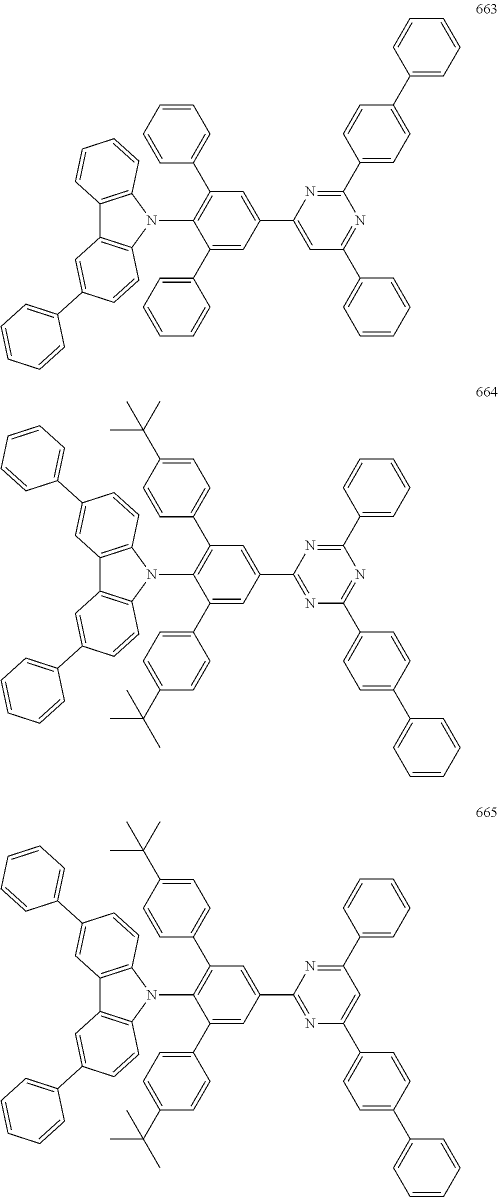 [SMILES: c1ccc(-c2ccc(-c3nc(-c4ccccc4)cc(-c4cc(-c5ccccc5)c(-n5c6ccccc6c6cc(-c7ccccc7)ccc65)c(-c5ccccc5)c4)n3)cc2)cc1 ; CC(C)(C)c1ccc(-c2cc(-c3nc(-c4ccccc4)nc(-c4ccc(-c5ccccc5)cc4)n3)cc(-c3ccc(C(C)(C)C)cc3)c2-n2c3ccc(-c4ccccc4)cc3c3cc(-c4ccccc4)ccc32)cc1 ; CC(C)(C)c1ccc(-c2cc(-c3nc(-c4ccccc4)cc(-c4ccc(-c5ccccc5)cc4)n3)cc(-c3ccc(C(C)(C)C)cc3)c2-n2c3ccc(-c4ccccc4)cc3c3cc(-c4ccccc4)ccc32)cc1]

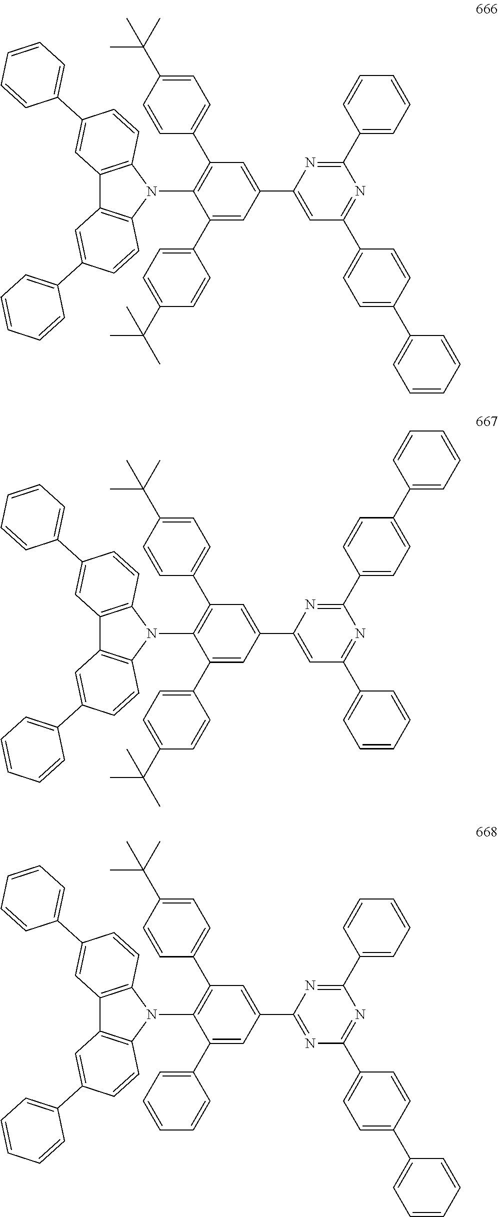

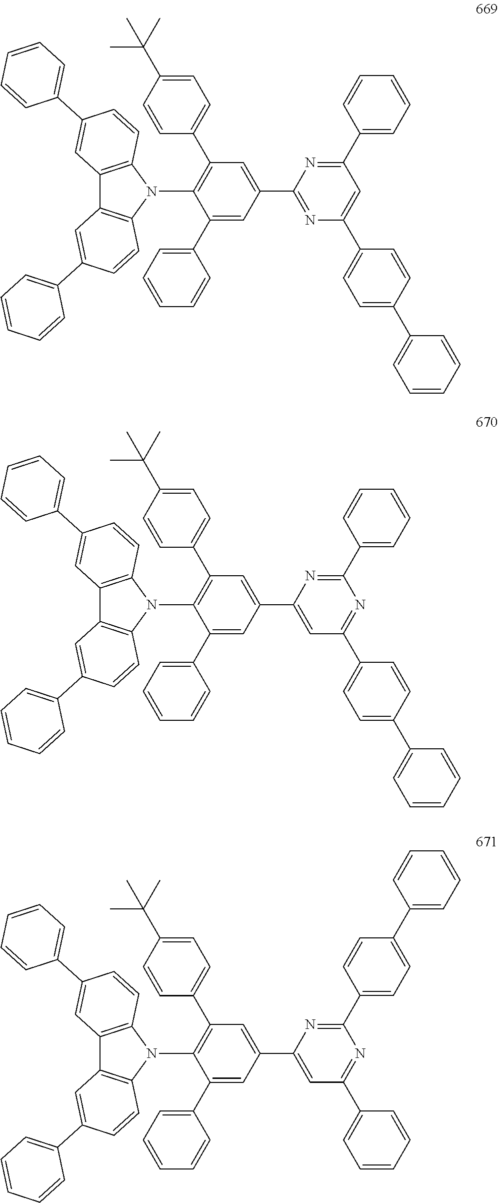

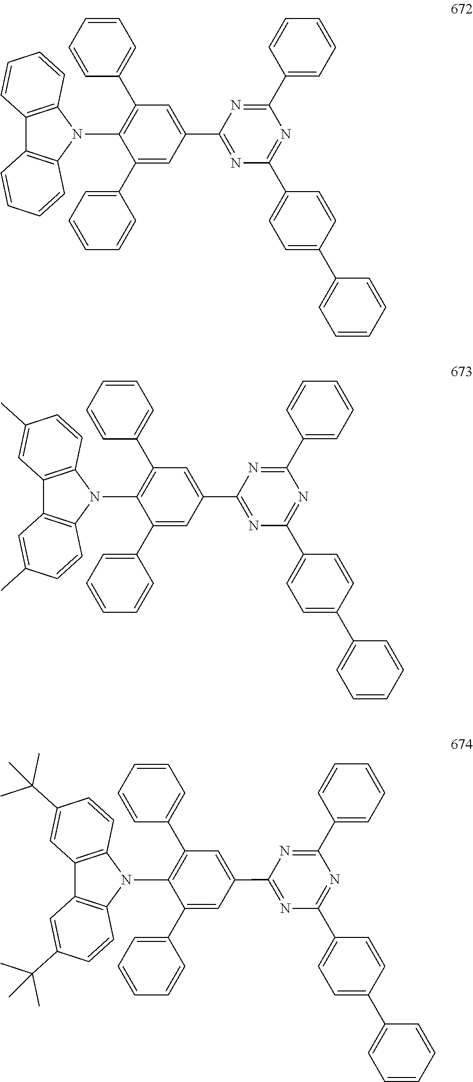

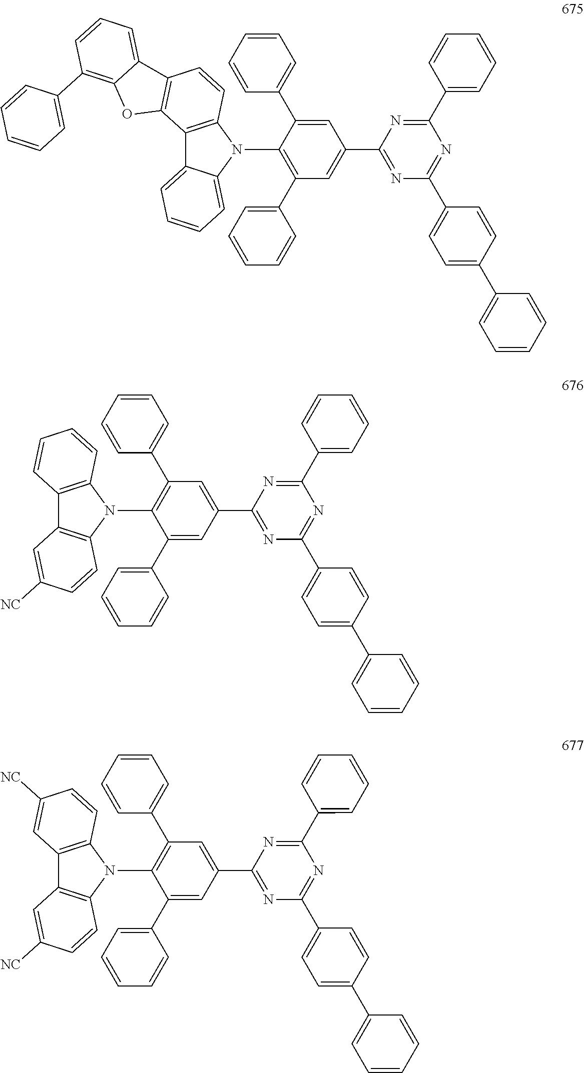

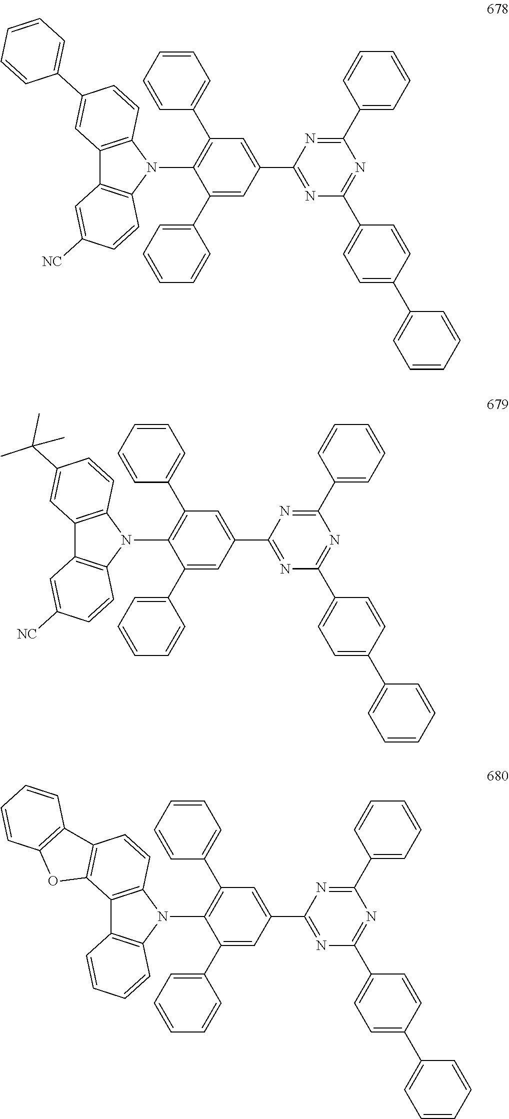 [SMILES: N#Cc1ccc2c(c1)c1cc(-c3ccccc3)ccc1n2-c1c(-c2ccccc2)cc(-c2nc(-c3ccccc3)nc(-c3ccc(-c4ccccc4)cc3)n2)cc1-c1ccccc1 ; CC(C)(C)c1ccc2c(c1)c1cc(C#N)ccc1n2-c1c(-c2ccccc2)cc(-c2nc(-c3ccccc3)nc(-c3ccc(-c4ccccc4)cc3)n2)cc1-c1ccccc1 ; c1ccc(-c2ccc(-c3nc(-c4ccccc4)nc(-c4cc(-c5ccccc5)c(-n5c6ccccc6c6c7oc8ccccc8c7ccc65)c(-c5ccccc5)c4)n3)cc2)cc1]

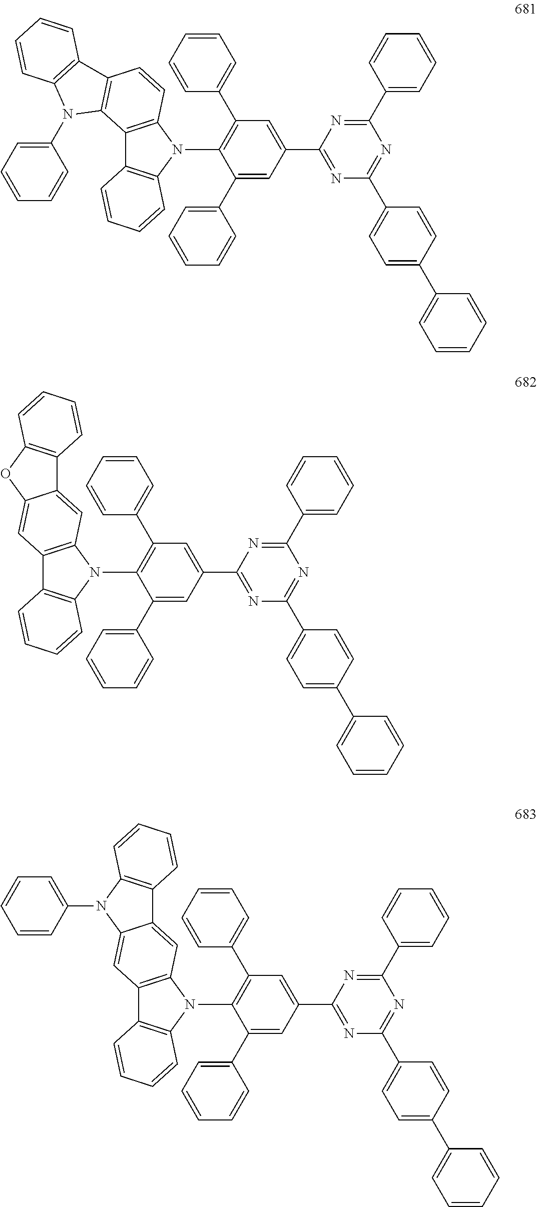 [SMILES: c1ccc(-c2ccc(-c3nc(-c4ccccc4)nc(-c4cc(-c5ccccc5)c(-n5c6ccccc6c6c5ccc5c7ccccc7n(-c7ccccc7)c56)c(-c5ccccc5)c4)n3)cc2)cc1 ; c1ccc(-c2ccc(-c3nc(-c4ccccc4)nc(-c4cc(-c5ccccc5)c(-n5c6ccccc6c6cc7oc8ccccc8c7cc65)c(-c5ccccc5)c4)n3)cc2)cc1 ; c1ccc(-c2ccc(-c3nc(-c4ccccc4)nc(-c4cc(-c5ccccc5)c(-n5c6ccccc6c6cc7c(cc65)c5ccccc5n7-c5ccccc5)c(-c5ccccc5)c4)n3)cc2)cc1]

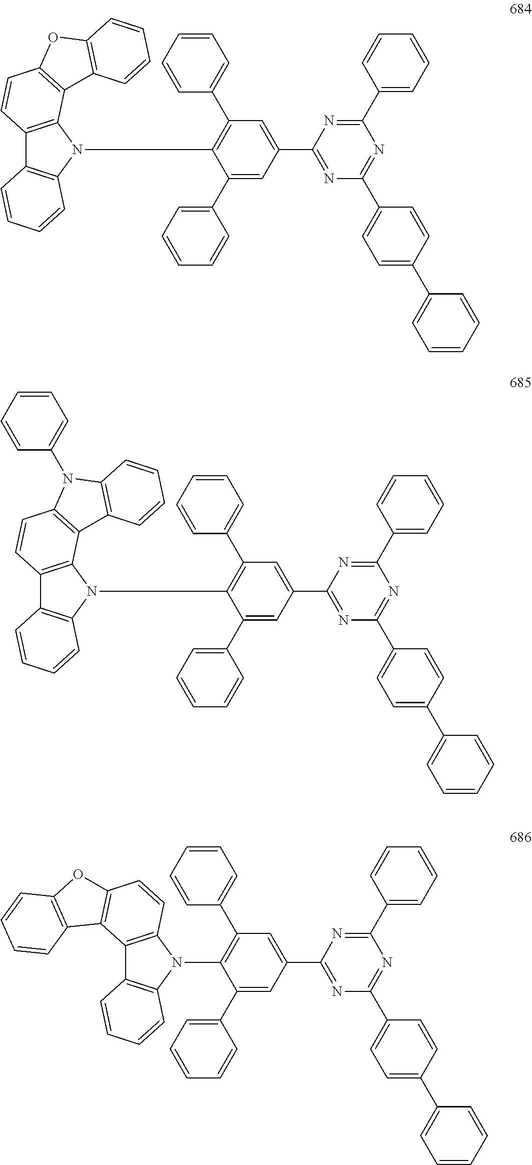 [SMILES: c1ccc(-c2ccc(-c3nc(-c4ccccc4)nc(-c4cc(-c5ccccc5)c(-n5c6ccccc6c6ccc7oc8ccccc8c7c65)c(-c5ccccc5)c4)n3)cc2)cc1 ; c1ccc(-c2ccc(-c3nc(-c4ccccc4)nc(-c4cc(-c5ccccc5)c(-n5c6ccccc6c6ccc7c(c8ccccc8n7-c7ccccc7)c65)c(-c5ccccc5)c4)n3)cc2)cc1 ; c1ccc(-c2ccc(-c3nc(-c4ccccc4)nc(-c4cc(-c5ccccc5)c(-n5c6ccccc6c6c7c(ccc65)oc5ccccc57)c(-c5ccccc5)c4)n3)cc2)cc1]

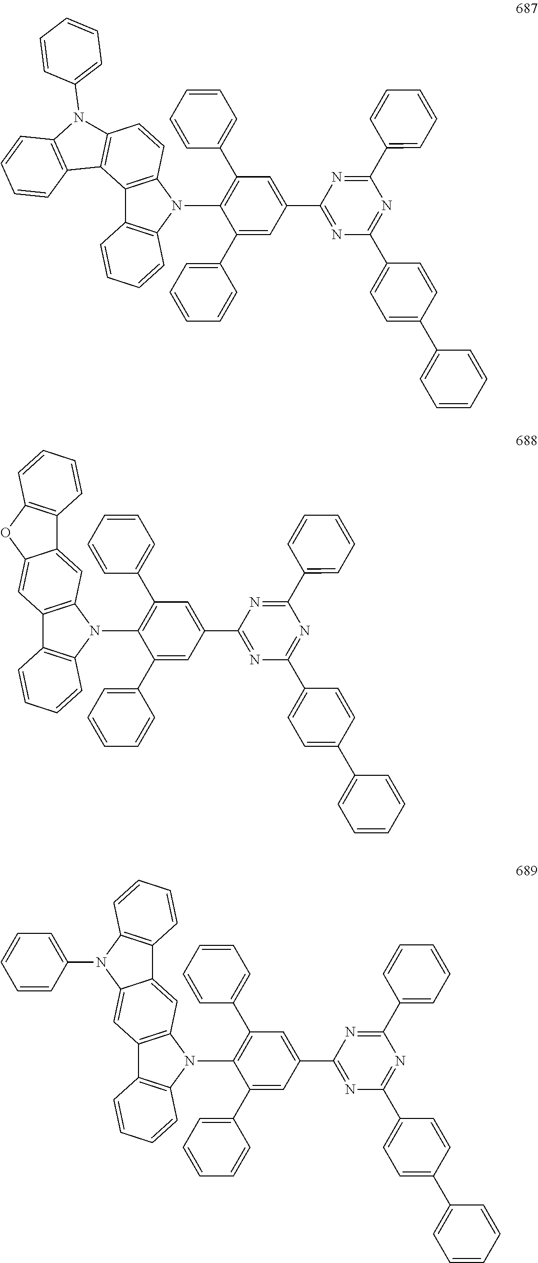 [SMILES: c1ccc(-c2ccc(-c3nc(-c4ccccc4)nc(-c4cc(-c5ccccc5)c(-n5c6ccccc6c6c7c8ccccc8n(-c8ccccc8)c7ccc65)c(-c5ccccc5)c4)n3)cc2)cc1 ; c1ccc(-c2ccc(-c3nc(-c4ccccc4)nc(-c4cc(-c5ccccc5)c(-n5c6ccccc6c6cc7oc8ccccc8c7cc65)c(-c5ccccc5)c4)n3)cc2)cc1 ; c1ccc(-c2ccc(-c3nc(-c4ccccc4)nc(-c4cc(-c5ccccc5)c(-n5c6ccccc6c6cc7c(cc65)c5ccccc5n7-c5ccccc5)c(-c5ccccc5)c4)n3)cc2)cc1]

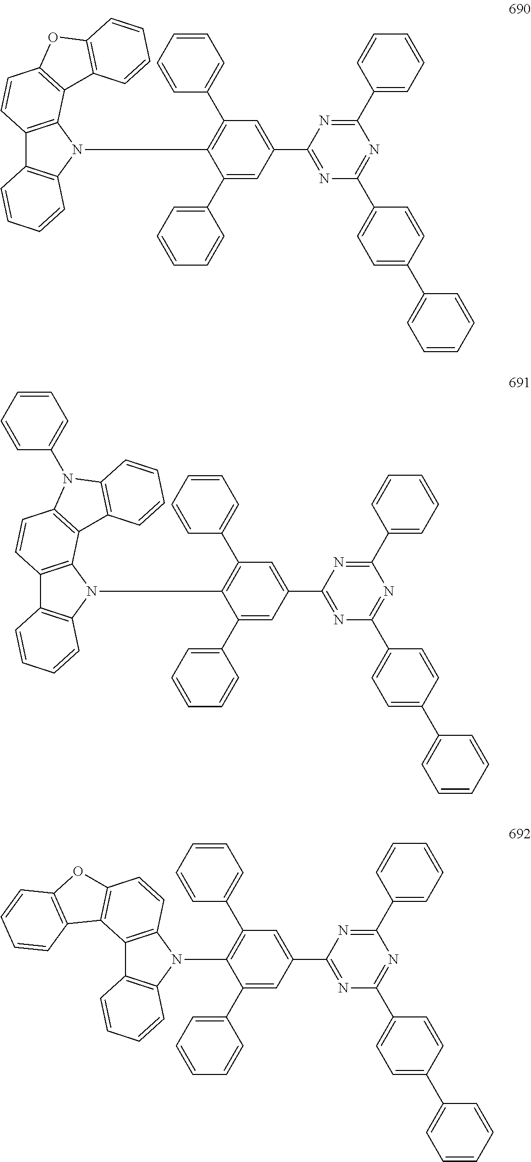 [SMILES: c1ccc(-c2ccc(-c3nc(-c4ccccc4)nc(-c4cc(-c5ccccc5)c(-n5c6ccccc6c6ccc7oc8ccccc8c7c65)c(-c5ccccc5)c4)n3)cc2)cc1 ; c1ccc(-c2ccc(-c3nc(-c4ccccc4)nc(-c4cc(-c5ccccc5)c(-n5c6ccccc6c6ccc7c(c8ccccc8n7-c7ccccc7)c65)c(-c5ccccc5)c4)n3)cc2)cc1 ; c1ccc(-c2ccc(-c3nc(-c4ccccc4)nc(-c4cc(-c5ccccc5)c(-n5c6ccccc6c6c7c(ccc65)oc5ccccc57)c(-c5ccccc5)c4)n3)cc2)cc1]

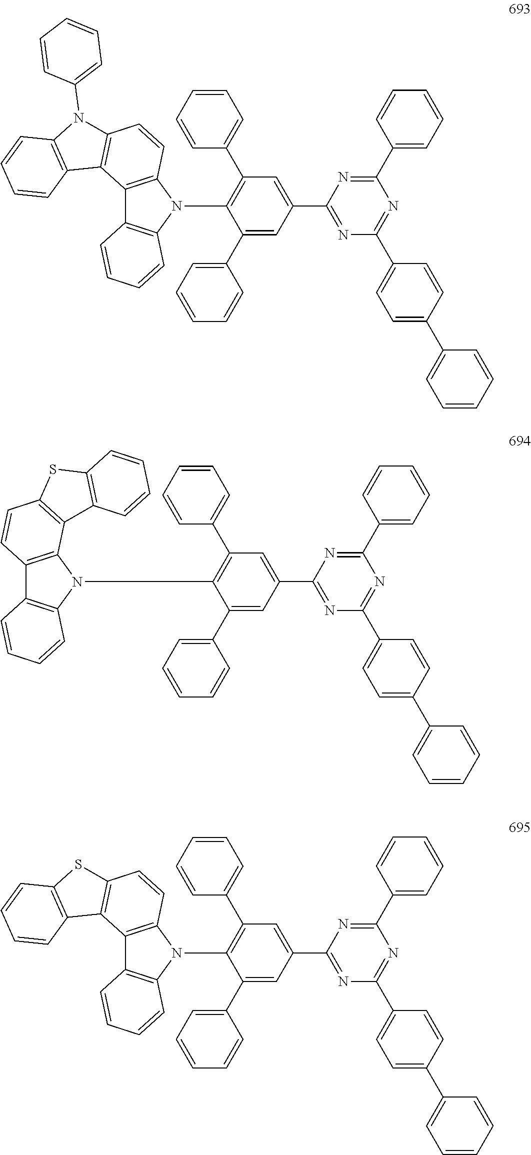 [SMILES: c1ccc(-c2ccc(-c3nc(-c4ccccc4)nc(-c4cc(-c5ccccc5)c(-n5c6ccccc6c6c7c8ccccc8n(-c8ccccc8)c7ccc65)c(-c5ccccc5)c4)n3)cc2)cc1 ; c1ccc(-c2ccc(-c3nc(-c4ccccc4)nc(-c4cc(-c5ccccc5)c(-n5c6ccccc6c6ccc7sc8ccccc8c7c65)c(-c5ccccc5)c4)n3)cc2)cc1 ; c1ccc(-c2ccc(-c3nc(-c4ccccc4)nc(-c4cc(-c5ccccc5)c(-n5c6ccccc6c6c7c(ccc65)sc5ccccc57)c(-c5ccccc5)c4)n3)cc2)cc1]

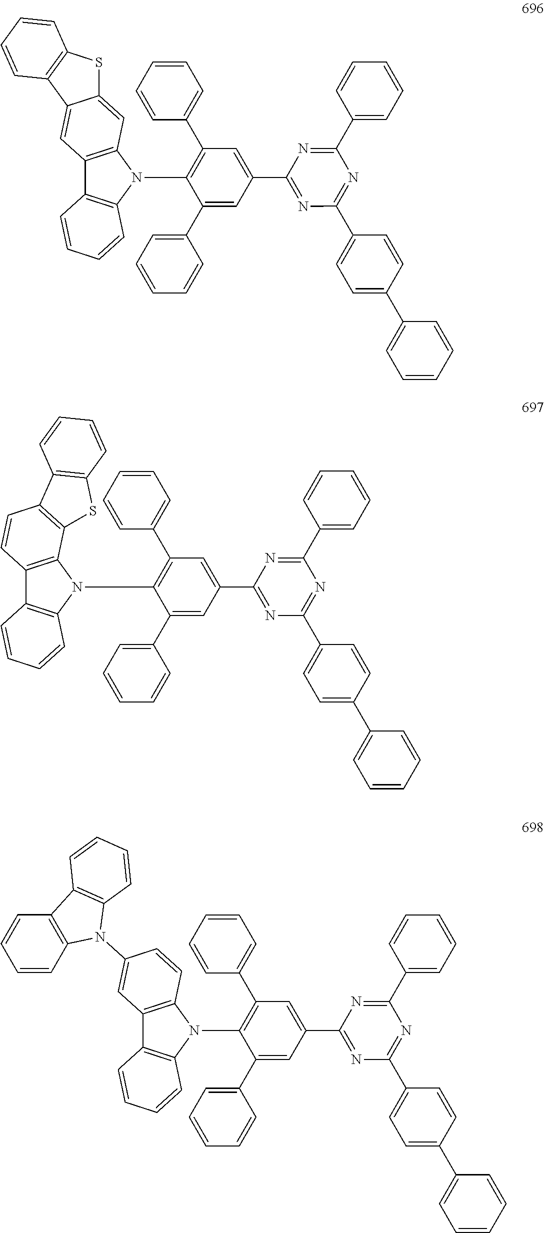 [SMILES: c1ccc(-c2ccc(-c3nc(-c4ccccc4)nc(-c4cc(-c5ccccc5)c(-n5c6ccccc6c6cc7c(cc65)sc5ccccc57)c(-c5ccccc5)c4)n3)cc2)cc1 ; c1ccc(-c2ccc(-c3nc(-c4ccccc4)nc(-c4cc(-c5ccccc5)c(-n5c6ccccc6c6ccc7c8ccccc8sc7c65)c(-c5ccccc5)c4)n3)cc2)cc1 ; c1ccc(-c2ccc(-c3nc(-c4ccccc4)nc(-c4cc(-c5ccccc5)c(-n5c6ccccc6c6cc(-n7c8ccccc8c8ccccc87)ccc65)c(-c5ccccc5)c4)n3)cc2)cc1]

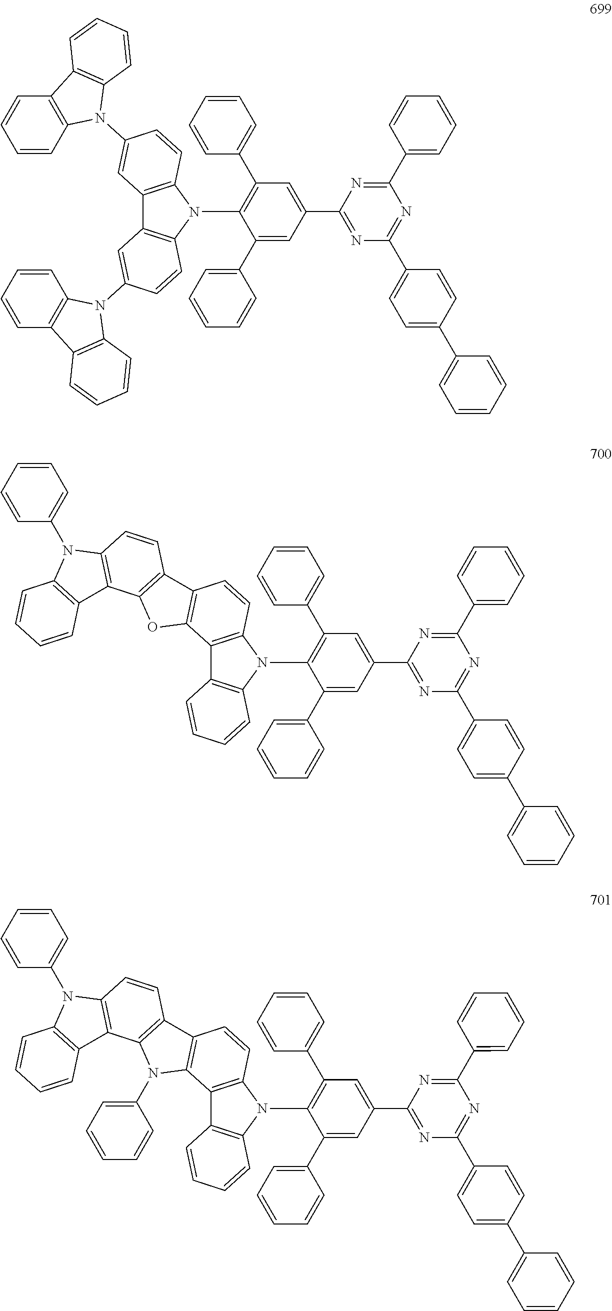 [SMILES: c1ccc(-c2ccc(-c3nc(-c4ccccc4)nc(-c4cc(-c5ccccc5)c(-n5c6ccc(-n7c8ccccc8c8ccccc87)cc6c6cc(-n7c8ccccc8c8ccccc87)ccc65)c(-c5ccccc5)c4)n3)cc2)cc1 ; c1ccc(-c2ccc(-c3nc(-c4ccccc4)nc(-c4cc(-c5ccccc5)c(-n5c6ccccc6c6c7oc8c(ccc9c8c8ccccc8n9-c8ccccc8)c7ccc65)c(-c5ccccc5)c4)n3)cc2)cc1 ; c1ccc(-c2ccc(-c3nc(-c4ccccc4)nc(-c4cc(-c5ccccc5)c(-n5c6ccccc6c6c5ccc5c7ccc8c(c9ccccc9n8-c8ccccc8)c7n(-c7ccccc7)c56)c(-c5ccccc5)c4)n3)cc2)cc1]

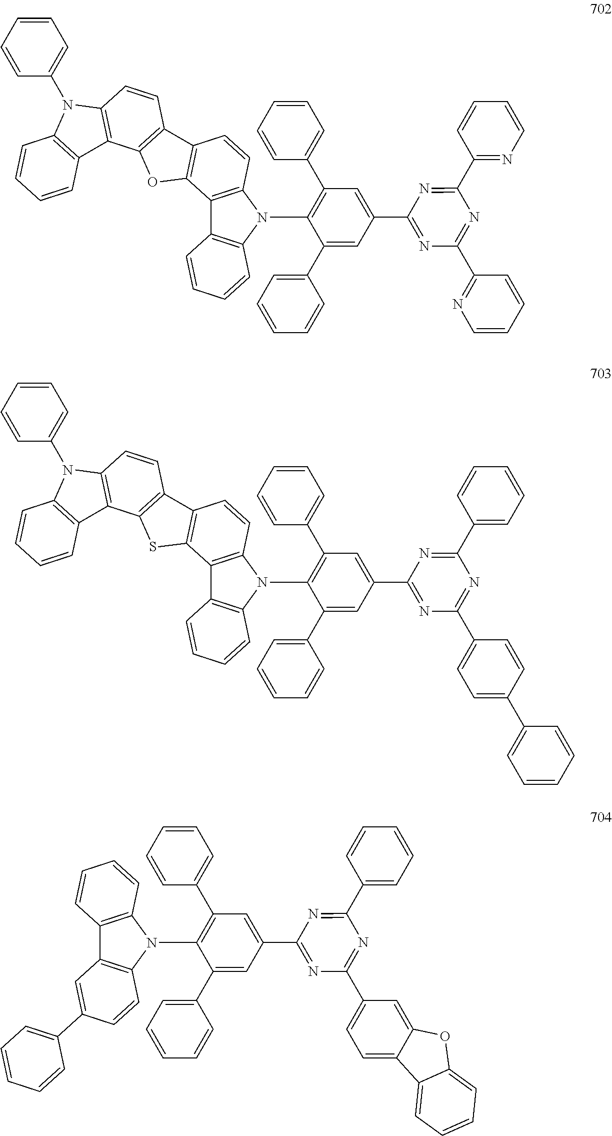 [SMILES: c1ccc(-c2cc(-c3nc(-c4ccccn4)nc(-c4ccccn4)n3)cc(-c3ccccc3)c2-n2c3ccccc3c3c4oc5c(ccc6c5c5ccccc5n6-c5ccccc5)c4ccc32)cc1 ; c1ccc(-c2ccc(-c3nc(-c4ccccc4)nc(-c4cc(-c5ccccc5)c(-n5c6ccccc6c6c7sc8c(ccc9c8c8ccccc8n9-c8ccccc8)c7ccc65)c(-c5ccccc5)c4)n3)cc2)cc1 ; c1ccc(-c2ccc3c(c2)c2ccccc2n3-c2c(-c3ccccc3)cc(-c3nc(-c4ccccc4)nc(-c4ccc5c(c4)oc4ccccc45)n3)cc2-c2ccccc2)cc1]

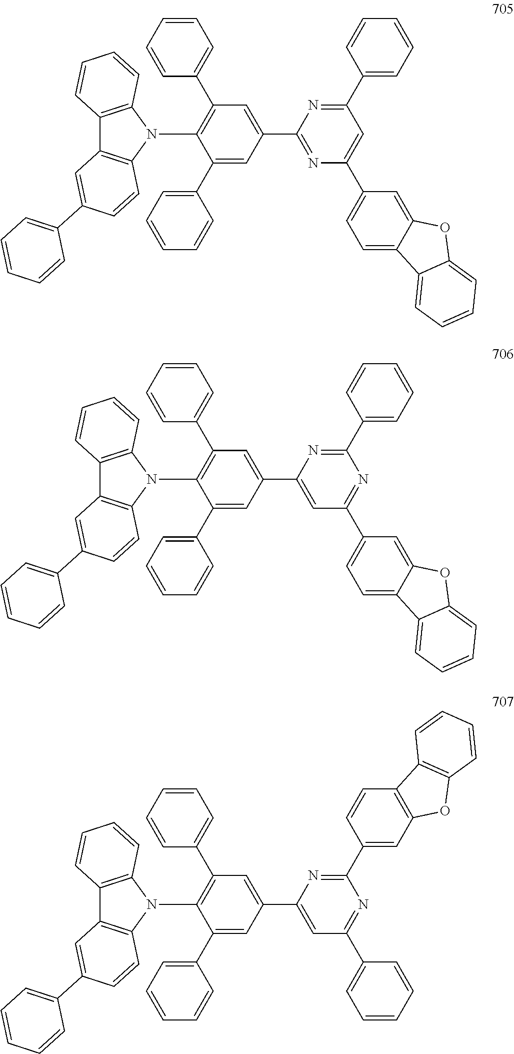

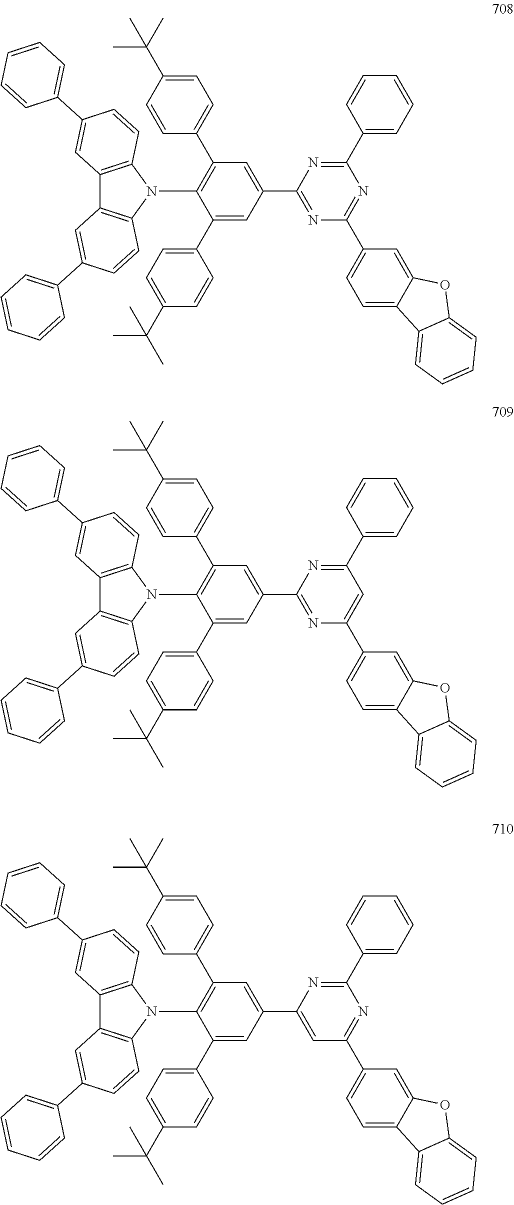 [SMILES: CC(C)(C)c1ccc(-c2cc(-c3nc(-c4ccccc4)nc(-c4ccc5c(c4)oc4ccccc45)n3)cc(-c3ccc(C(C)(C)C)cc3)c2-n2c3ccc(-c4ccccc4)cc3c3cc(-c4ccccc4)ccc32)cc1 ; CC(C)(C)c1ccc(-c2cc(-c3nc(-c4ccccc4)cc(-c4ccc5c(c4)oc4ccccc45)n3)cc(-c3ccc(C(C)(C)C)cc3)c2-n2c3ccc(-c4ccccc4)cc3c3cc(-c4ccccc4)ccc32)cc1 ; CC(C)(C)c1ccc(-c2cc(-c3cc(-c4ccc5c(c4)oc4ccccc45)nc(-c4ccccc4)n3)cc(-c3ccc(C(C)(C)C)cc3)c2-n2c3ccc(-c4ccccc4)cc3c3cc(-c4ccccc4)ccc32)cc1]

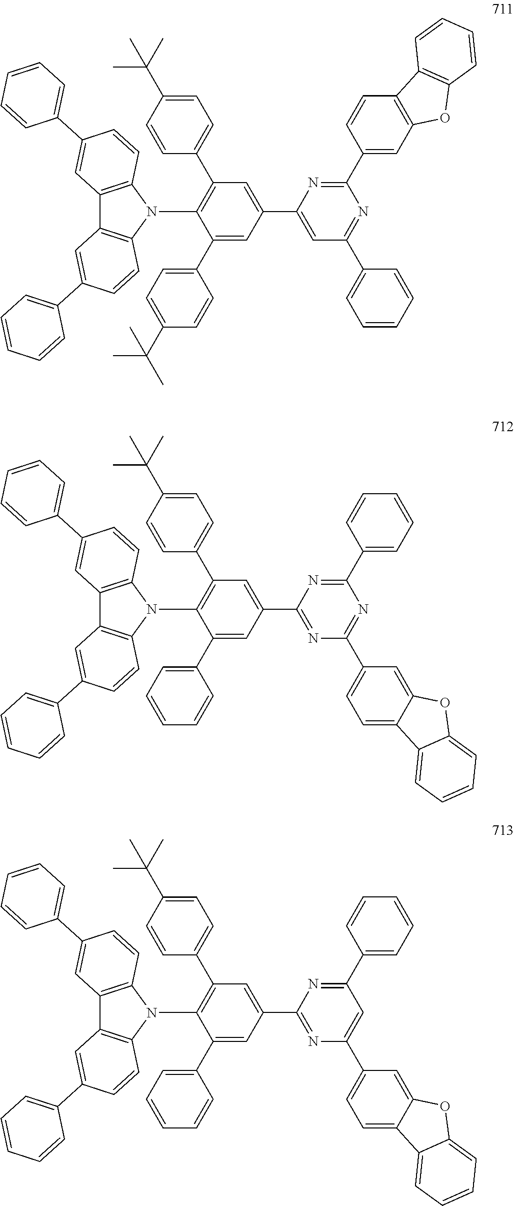 [SMILES: CC(C)(C)c1ccc(-c2cc(-c3cc(-c4ccccc4)nc(-c4ccc5c(c4)oc4ccccc45)n3)cc(-c3ccc(C(C)(C)C)cc3)c2-n2c3ccc(-c4ccccc4)cc3c3cc(-c4ccccc4)ccc32)cc1 ; CC(C)(C)c1ccc(-c2cc(-c3nc(-c4ccccc4)nc(-c4ccc5c(c4)oc4ccccc45)n3)cc(-c3ccccc3)c2-n2c3ccc(-c4ccccc4)cc3c3cc(-c4ccccc4)ccc32)cc1 ; CC(C)(C)c1ccc(-c2cc(-c3nc(-c4ccccc4)cc(-c4ccc5c(c4)oc4ccccc45)n3)cc(-c3ccccc3)c2-n2c3ccc(-c4ccccc4)cc3c3cc(-c4ccccc4)ccc32)cc1]

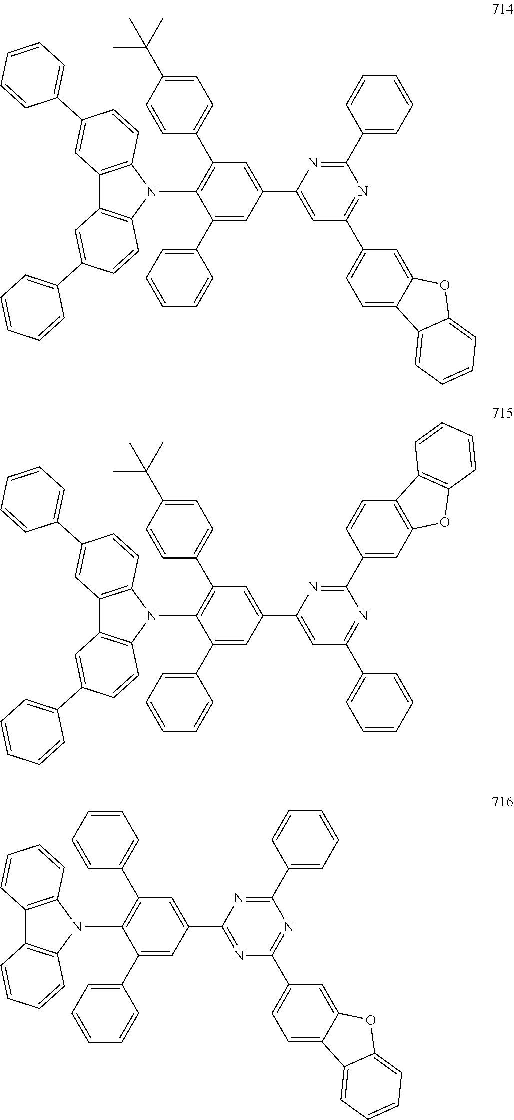

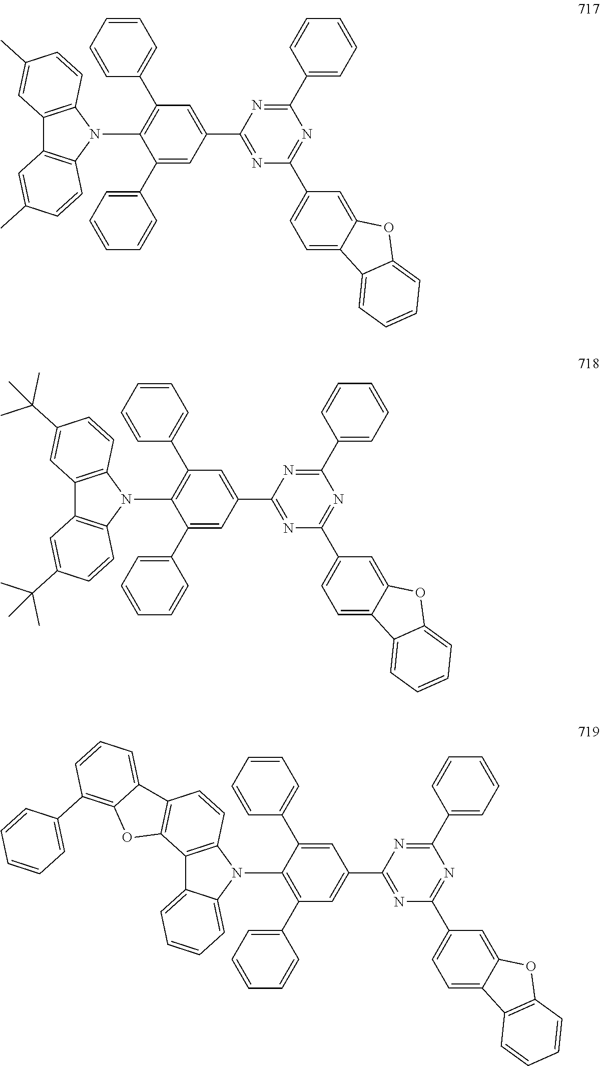 [SMILES: Cc1ccc2c(c1)c1cc(C)ccc1n2-c1c(-c2ccccc2)cc(-c2nc(-c3ccccc3)nc(-c3ccc4c(c3)oc3ccccc34)n2)cc1-c1ccccc1 ; CC(C)(C)c1ccc2c(c1)c1cc(C(C)(C)C)ccc1n2-c1c(-c2ccccc2)cc(-c2nc(-c3ccccc3)nc(-c3ccc4c(c3)oc3ccccc34)n2)cc1-c1ccccc1 ; c1ccc(-c2nc(-c3cc(-c4ccccc4)c(-n4c5ccccc5c5c6oc7c(-c8ccccc8)cccc7c6ccc54)c(-c4ccccc4)c3)nc(-c3ccc4c(c3)oc3ccccc34)n2)cc1]

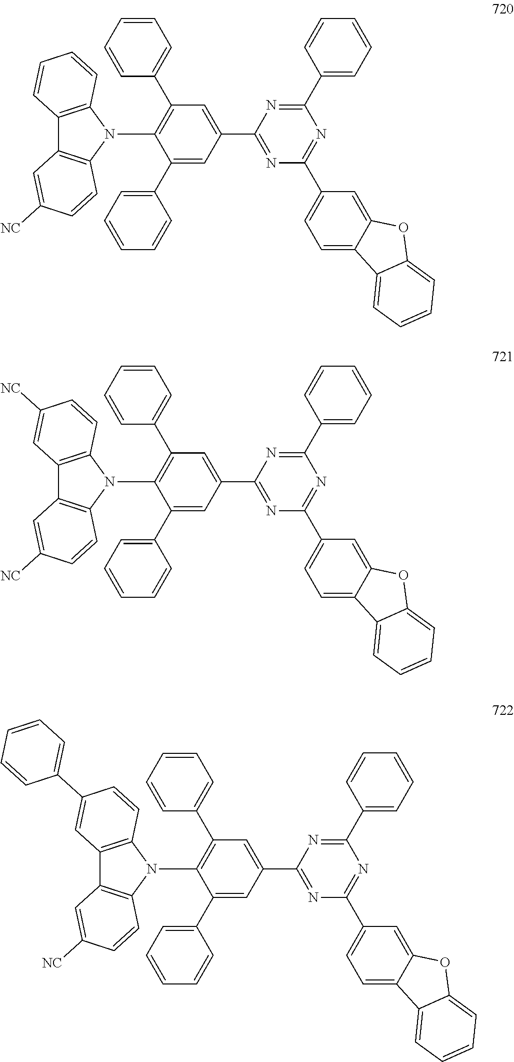 [SMILES: N#Cc1ccc2c(c1)c1ccccc1n2-c1c(-c2ccccc2)cc(-c2nc(-c3ccccc3)nc(-c3ccc4c(c3)oc3ccccc34)n2)cc1-c1ccccc1 ; N#Cc1ccc2c(c1)c1cc(C#N)ccc1n2-c1c(-c2ccccc2)cc(-c2nc(-c3ccccc3)nc(-c3ccc4c(c3)oc3ccccc34)n2)cc1-c1ccccc1 ; N#Cc1ccc2c(c1)c1cc(-c3ccccc3)ccc1n2-c1c(-c2ccccc2)cc(-c2nc(-c3ccccc3)nc(-c3ccc4c(c3)oc3ccccc34)n2)cc1-c1ccccc1]

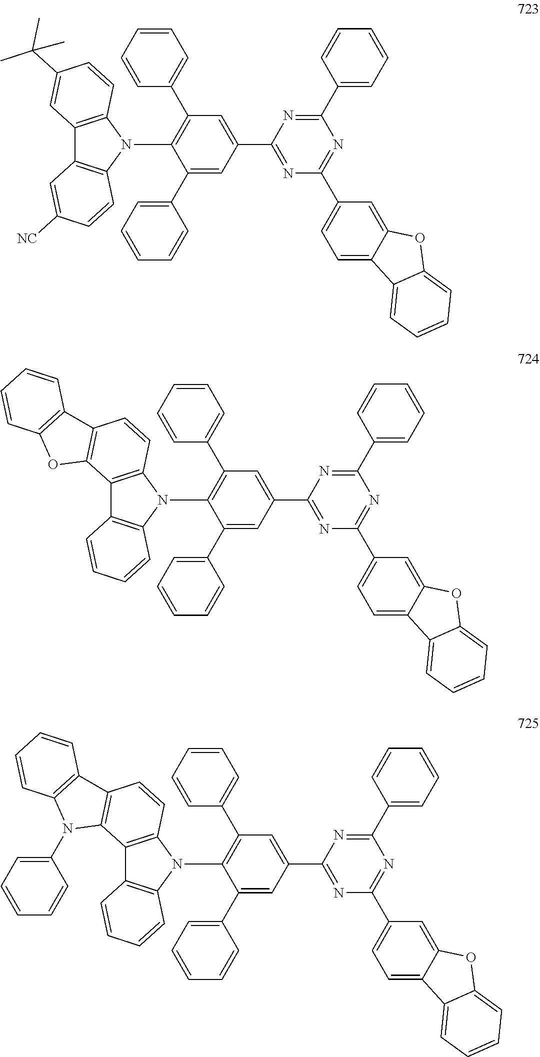 [SMILES: CC(C)(C)c1ccc2c(c1)c1cc(C#N)ccc1n2-c1c(-c2ccccc2)cc(-c2nc(-c3ccccc3)nc(-c3ccc4c(c3)oc3ccccc34)n2)cc1-c1ccccc1 ; c1ccc(-c2nc(-c3cc(-c4ccccc4)c(-n4c5ccccc5c5c6oc7ccccc7c6ccc54)c(-c4ccccc4)c3)nc(-c3ccc4c(c3)oc3ccccc34)n2)cc1 ; c1ccc(-c2nc(-c3cc(-c4ccccc4)c(-n4c5ccccc5c5c4ccc4c6ccccc6n(-c6ccccc6)c45)c(-c4ccccc4)c3)nc(-c3ccc4c(c3)oc3ccccc34)n2)cc1]

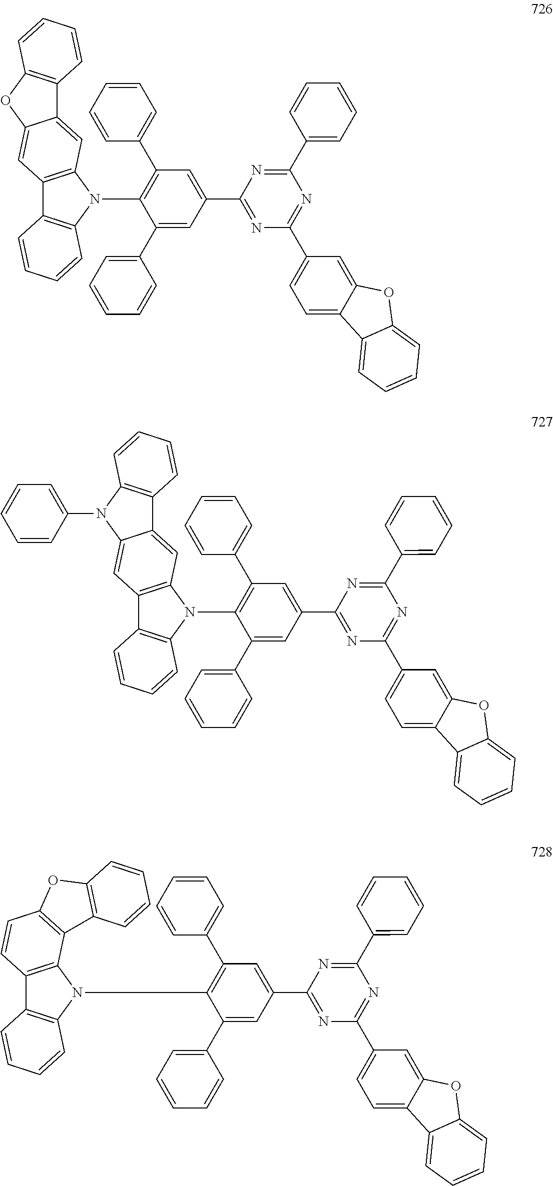 [SMILES: c1ccc(-c2nc(-c3cc(-c4ccccc4)c(-n4c5ccccc5c5cc6oc7ccccc7c6cc54)c(-c4ccccc4)c3)nc(-c3ccc4c(c3)oc3ccccc34)n2)cc1 ; c1ccc(-c2nc(-c3cc(-c4ccccc4)c(-n4c5ccccc5c5cc6c(cc54)c4ccccc4n6-c4ccccc4)c(-c4ccccc4)c3)nc(-c3ccc4c(c3)oc3ccccc34)n2)cc1 ; c1ccc(-c2nc(-c3cc(-c4ccccc4)c(-n4c5ccccc5c5ccc6oc7ccccc7c6c54)c(-c4ccccc4)c3)nc(-c3ccc4c(c3)oc3ccccc34)n2)cc1]

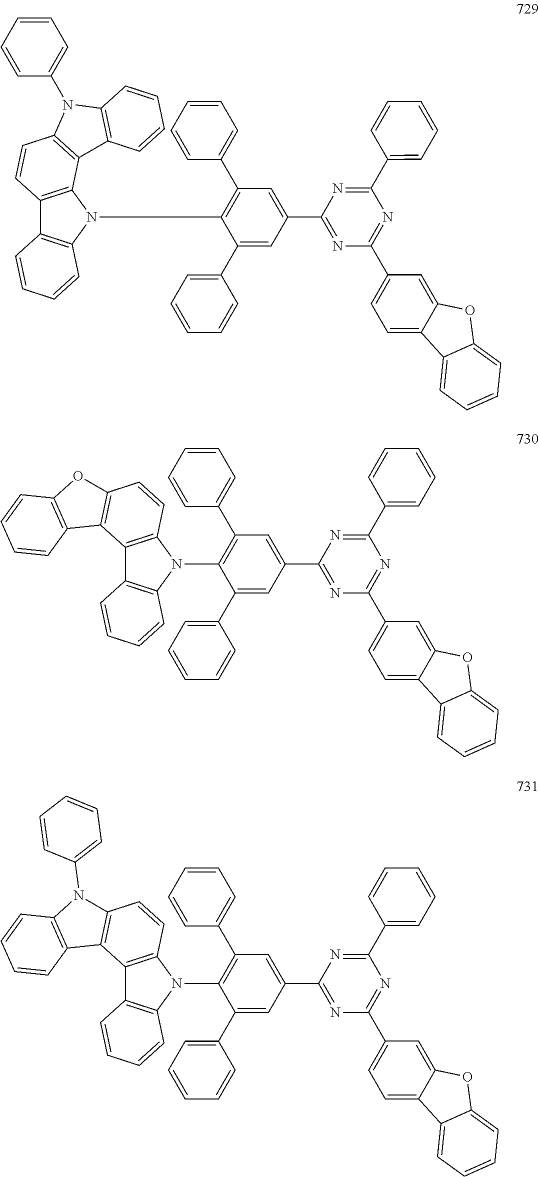 [SMILES: c1ccc(-c2nc(-c3cc(-c4ccccc4)c(-n4c5ccccc5c5ccc6c(c7ccccc7n6-c6ccccc6)c54)c(-c4ccccc4)c3)nc(-c3ccc4c(c3)oc3ccccc34)n2)cc1 ; c1ccc(-c2nc(-c3cc(-c4ccccc4)c(-n4c5ccccc5c5c6c(ccc54)oc4ccccc46)c(-c4ccccc4)c3)nc(-c3ccc4c(c3)oc3ccccc34)n2)cc1 ; c1ccc(-c2nc(-c3cc(-c4ccccc4)c(-n4c5ccccc5c5c6c7ccccc7n(-c7ccccc7)c6ccc54)c(-c4ccccc4)c3)nc(-c3ccc4c(c3)oc3ccccc34)n2)cc1]

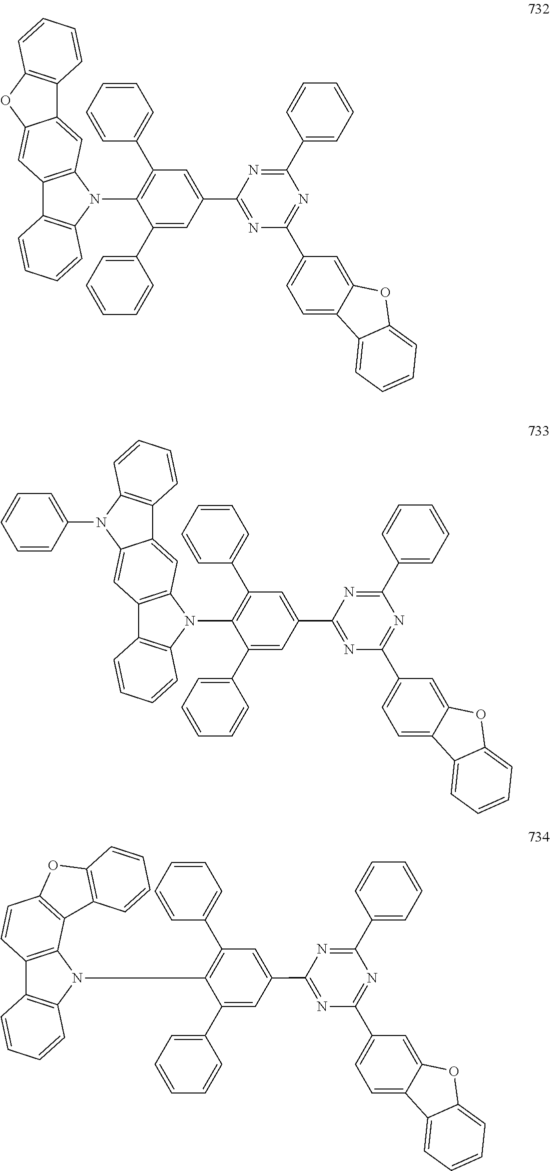 [SMILES: c1ccc(-c2nc(-c3cc(-c4ccccc4)c(-n4c5ccccc5c5cc6oc7ccccc7c6cc54)c(-c4ccccc4)c3)nc(-c3ccc4c(c3)oc3ccccc34)n2)cc1 ; c1ccc(-c2nc(-c3cc(-c4ccccc4)c(-n4c5ccccc5c5cc6c(cc54)c4ccccc4n6-c4ccccc4)c(-c4ccccc4)c3)nc(-c3ccc4c(c3)oc3ccccc34)n2)cc1 ; c1ccc(-c2nc(-c3cc(-c4ccccc4)c(-n4c5ccccc5c5ccc6oc7ccccc7c6c54)c(-c4ccccc4)c3)nc(-c3ccc4c(c3)oc3ccccc34)n2)cc1]

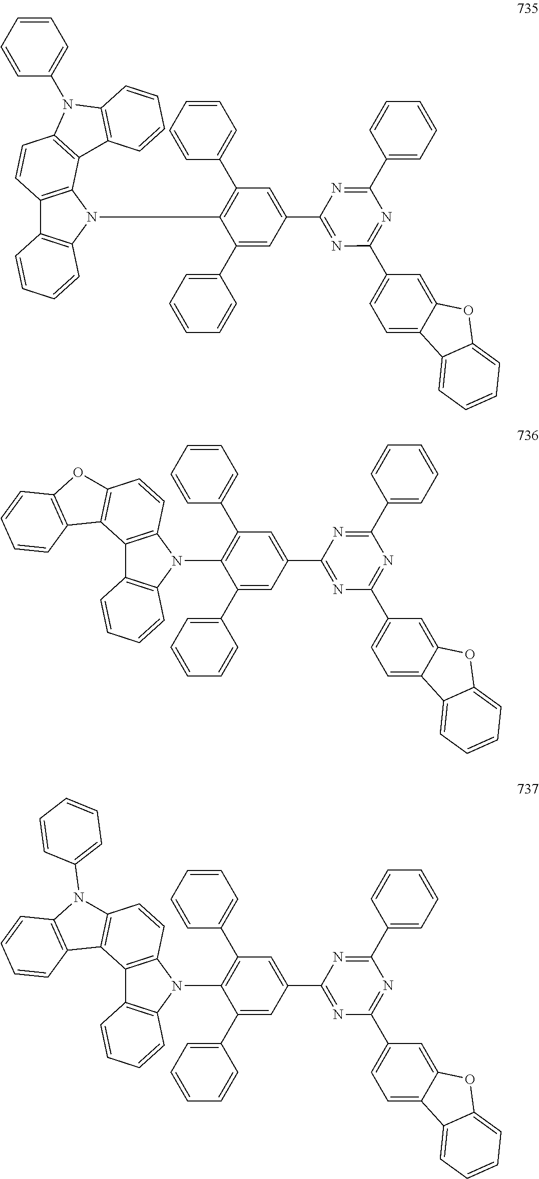 [SMILES: c1ccc(-c2nc(-c3cc(-c4ccccc4)c(-n4c5ccccc5c5ccc6c(c7ccccc7n6-c6ccccc6)c54)c(-c4ccccc4)c3)nc(-c3ccc4c(c3)oc3ccccc34)n2)cc1 ; c1ccc(-c2nc(-c3cc(-c4ccccc4)c(-n4c5ccccc5c5c6c(ccc54)oc4ccccc46)c(-c4ccccc4)c3)nc(-c3ccc4c(c3)oc3ccccc34)n2)cc1 ; c1ccc(-c2nc(-c3cc(-c4ccccc4)c(-n4c5ccccc5c5c6c7ccccc7n(-c7ccccc7)c6ccc54)c(-c4ccccc4)c3)nc(-c3ccc4c(c3)oc3ccccc34)n2)cc1]

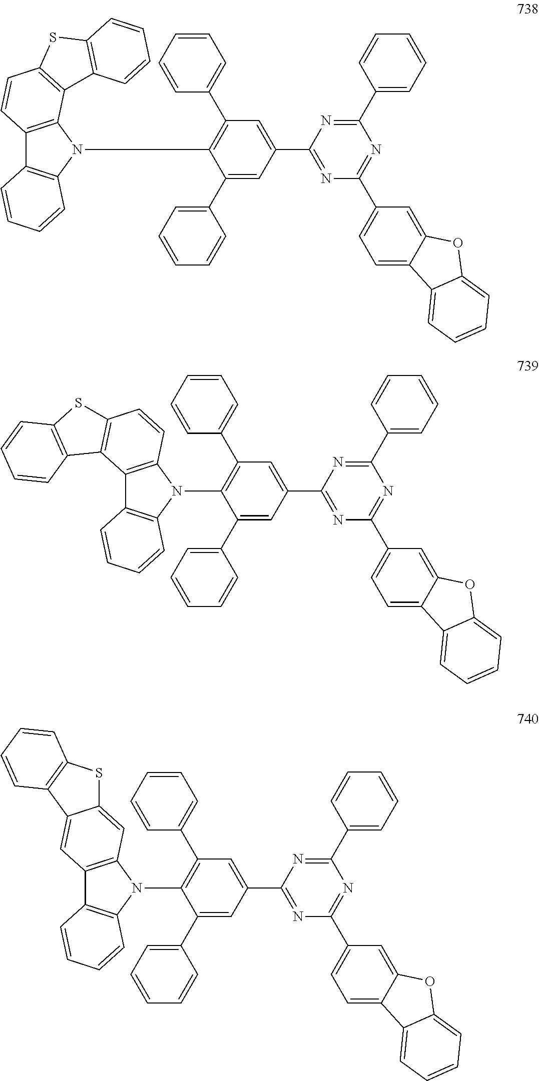 [SMILES: c1ccc(-c2nc(-c3cc(-c4ccccc4)c(-n4c5ccccc5c5ccc6sc7ccccc7c6c54)c(-c4ccccc4)c3)nc(-c3ccc4c(c3)oc3ccccc34)n2)cc1 ; c1ccc(-c2nc(-c3cc(-c4ccccc4)c(-n4c5ccccc5c5c6c(ccc54)sc4ccccc46)c(-c4ccccc4)c3)nc(-c3ccc4c(c3)oc3ccccc34)n2)cc1 ; c1ccc(-c2nc(-c3cc(-c4ccccc4)c(-n4c5ccccc5c5cc6c(cc54)sc4ccccc46)c(-c4ccccc4)c3)nc(-c3ccc4c(c3)oc3ccccc34)n2)cc1]

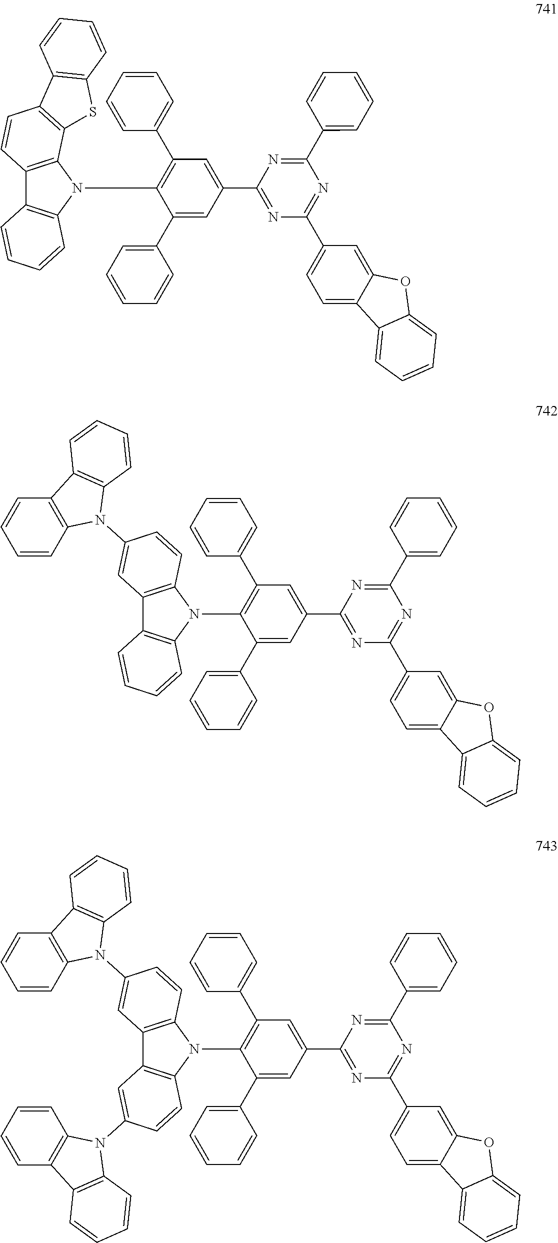 [SMILES: c1ccc(-c2nc(-c3cc(-c4ccccc4)c(-n4c5ccccc5c5ccc6c7ccccc7sc6c54)c(-c4ccccc4)c3)nc(-c3ccc4c(c3)oc3ccccc34)n2)cc1 ; c1ccc(-c2nc(-c3cc(-c4ccccc4)c(-n4c5ccccc5c5cc(-n6c7ccccc7c7ccccc76)ccc54)c(-c4ccccc4)c3)nc(-c3ccc4c(c3)oc3ccccc34)n2)cc1 ; c1ccc(-c2nc(-c3cc(-c4ccccc4)c(-n4c5ccc(-n6c7ccccc7c7ccccc76)cc5c5cc(-n6c7ccccc7c7ccccc76)ccc54)c(-c4ccccc4)c3)nc(-c3ccc4c(c3)oc3ccccc34)n2)cc1]

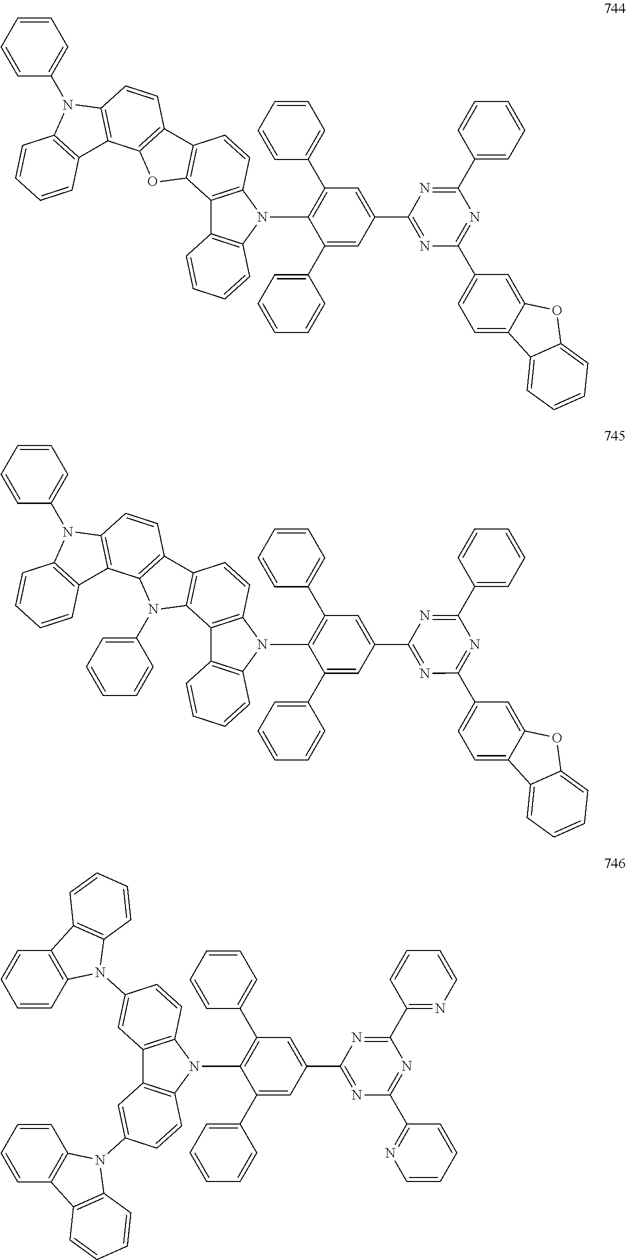 [SMILES: c1ccc(-c2nc(-c3cc(-c4ccccc4)c(-n4c5ccccc5c5c6oc7c(ccc8c7c7ccccc7n8-c7ccccc7)c6ccc54)c(-c4ccccc4)c3)nc(-c3ccc4c(c3)oc3ccccc34)n2)cc1 ; c1ccc(-c2nc(-c3cc(-c4ccccc4)c(-n4c5ccccc5c5c4ccc4c6ccc7c(c8ccccc8n7-c7ccccc7)c6n(-c6ccccc6)c45)c(-c4ccccc4)c3)nc(-c3ccc4c(c3)oc3ccccc34)n2)cc1 ; c1ccc(-c2cc(-c3nc(-c4ccccn4)nc(-c4ccccn4)n3)cc(-c3ccccc3)c2-n2c3ccc(-n4c5ccccc5c5ccccc54)cc3c3cc(-n4c5ccccc5c5ccccc54)ccc32)cc1]

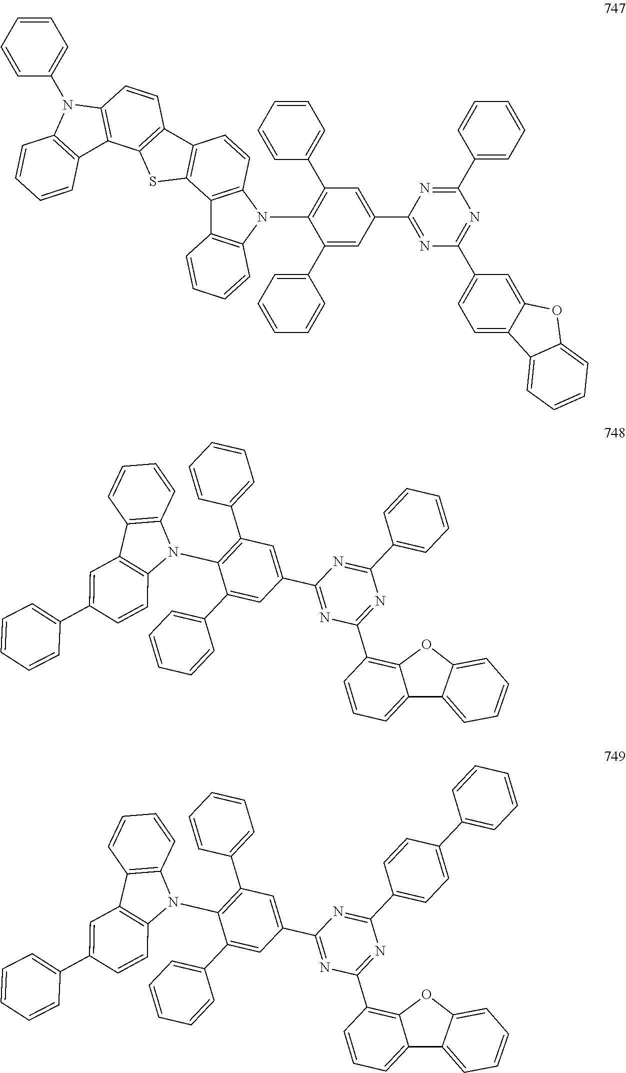

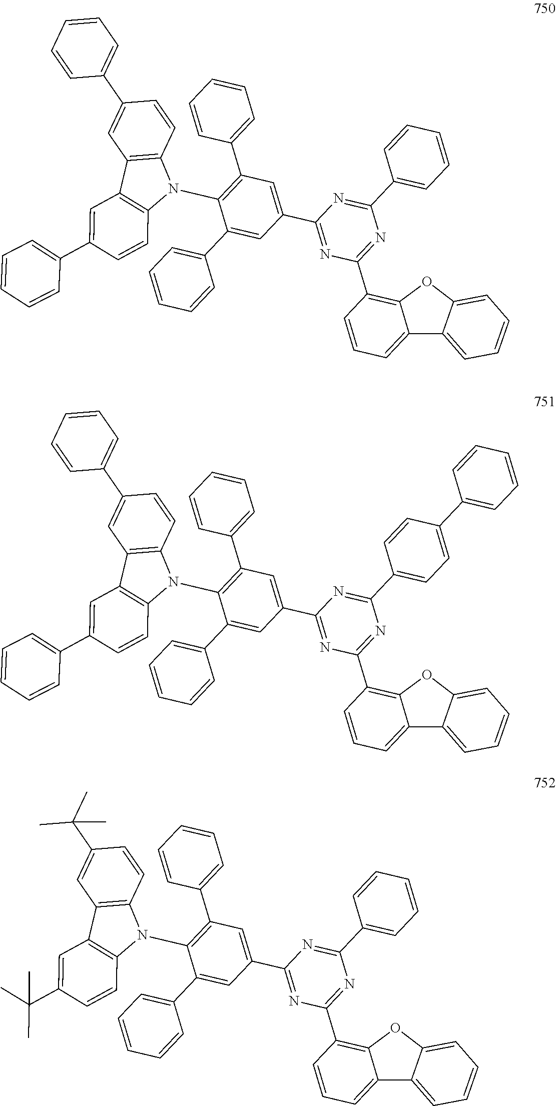 [SMILES: c1ccc(-c2ccc3c(c2)c2cc(-c4ccccc4)ccc2n3-c2c(-c3ccccc3)cc(-c3nc(-c4ccccc4)nc(-c4cccc5c4oc4ccccc45)n3)cc2-c2ccccc2)cc1 ; c1ccc(-c2ccc(-c3nc(-c4cc(-c5ccccc5)c(-n5c6ccc(-c7ccccc7)cc6c6cc(-c7ccccc7)ccc65)c(-c5ccccc5)c4)nc(-c4cccc5c4oc4ccccc45)n3)cc2)cc1 ; CC(C)(C)c1ccc2c(c1)c1cc(C(C)(C)C)ccc1n2-c1c(-c2ccccc2)cc(-c2nc(-c3ccccc3)nc(-c3cccc4c3oc3ccccc34)n2)cc1-c1ccccc1]

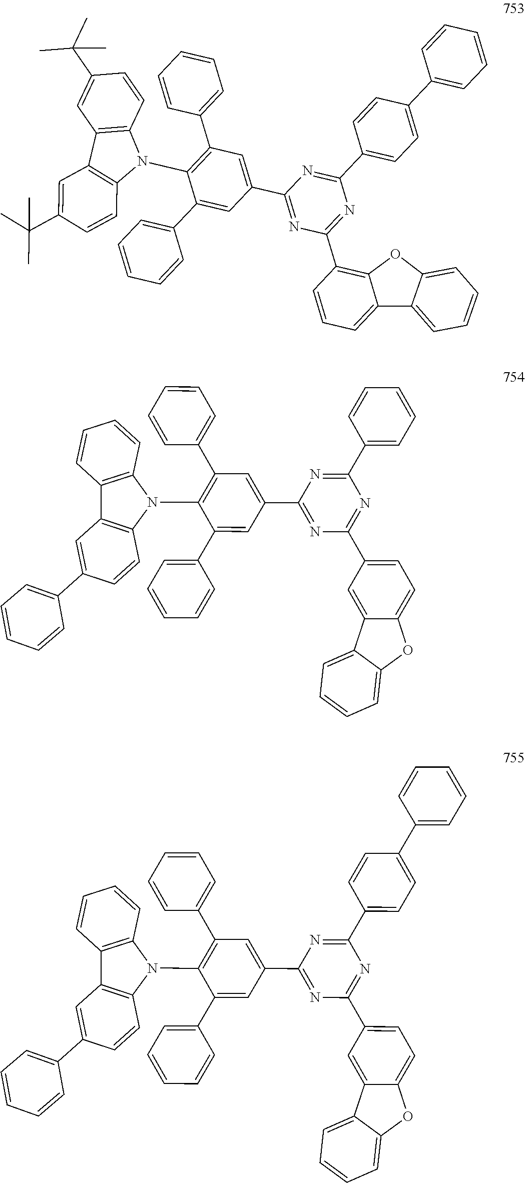 [SMILES: CC(C)(C)c1ccc2c(c1)c1cc(C(C)(C)C)ccc1n2-c1c(-c2ccccc2)cc(-c2nc(-c3ccc(-c4ccccc4)cc3)nc(-c3cccc4c3oc3ccccc34)n2)cc1-c1ccccc1 ; c1ccc(-c2ccc3c(c2)c2ccccc2n3-c2c(-c3ccccc3)cc(-c3nc(-c4ccccc4)nc(-c4ccc5oc6ccccc6c5c4)n3)cc2-c2ccccc2)cc1 ; c1ccc(-c2ccc(-c3nc(-c4cc(-c5ccccc5)c(-n5c6ccccc6c6cc(-c7ccccc7)ccc65)c(-c5ccccc5)c4)nc(-c4ccc5oc6ccccc6c5c4)n3)cc2)cc1]

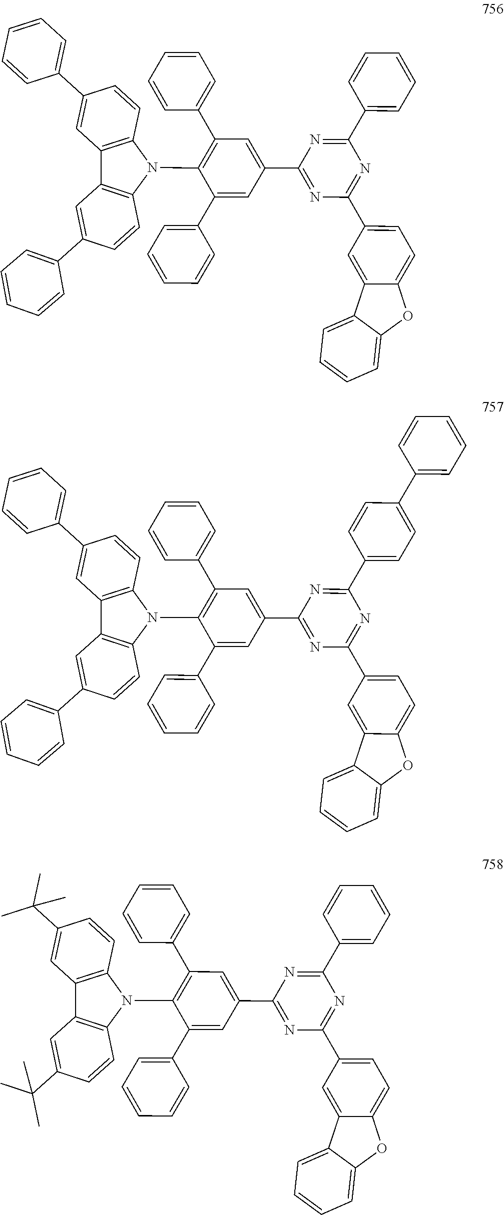 [SMILES: c1ccc(-c2ccc3c(c2)c2cc(-c4ccccc4)ccc2n3-c2c(-c3ccccc3)cc(-c3nc(-c4ccccc4)nc(-c4ccc5oc6ccccc6c5c4)n3)cc2-c2ccccc2)cc1 ; c1ccc(-c2ccc(-c3nc(-c4cc(-c5ccccc5)c(-n5c6ccc(-c7ccccc7)cc6c6cc(-c7ccccc7)ccc65)c(-c5ccccc5)c4)nc(-c4ccc5oc6ccccc6c5c4)n3)cc2)cc1 ; CC(C)(C)c1ccc2c(c1)c1cc(C(C)(C)C)ccc1n2-c1c(-c2ccccc2)cc(-c2nc(-c3ccccc3)nc(-c3ccc4oc5ccccc5c4c3)n2)cc1-c1ccccc1]

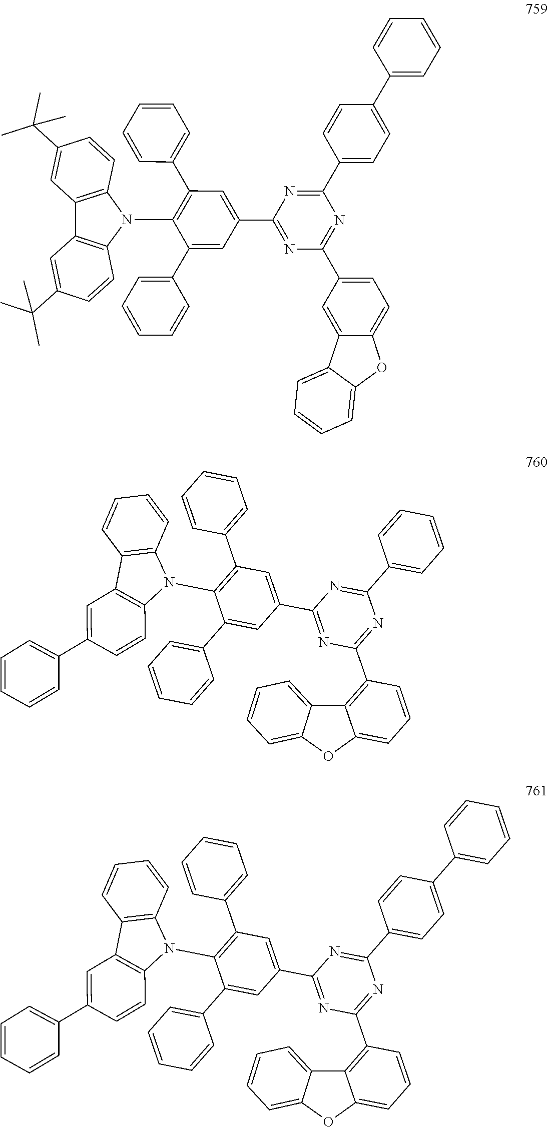 [SMILES: CC(C)(C)c1ccc2c(c1)c1cc(C(C)(C)C)ccc1n2-c1c(-c2ccccc2)cc(-c2nc(-c3ccc(-c4ccccc4)cc3)nc(-c3ccc4oc5ccccc5c4c3)n2)cc1-c1ccccc1 ; c1ccc(-c2ccc3c(c2)c2ccccc2n3-c2c(-c3ccccc3)cc(-c3nc(-c4ccccc4)nc(-c4cccc5oc6ccccc6c45)n3)cc2-c2ccccc2)cc1 ; c1ccc(-c2ccc(-c3nc(-c4cc(-c5ccccc5)c(-n5c6ccccc6c6cc(-c7ccccc7)ccc65)c(-c5ccccc5)c4)nc(-c4cccc5oc6ccccc6c45)n3)cc2)cc1]

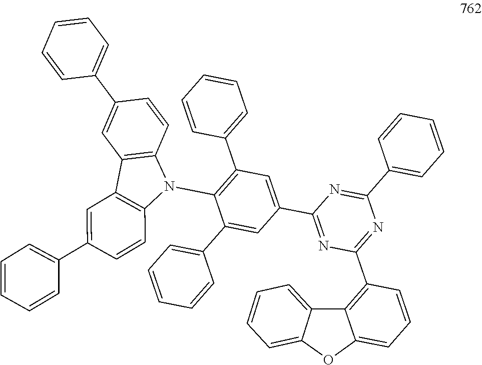 [SMILES: c1ccc(-c2ccc3c(c2)c2cc(-c4ccccc4)ccc2n3-c2c(-c3ccccc3)cc(-c3nc(-c4ccccc4)nc(-c4cccc5oc6ccccc6c45)n3)cc2-c2ccccc2)cc1]

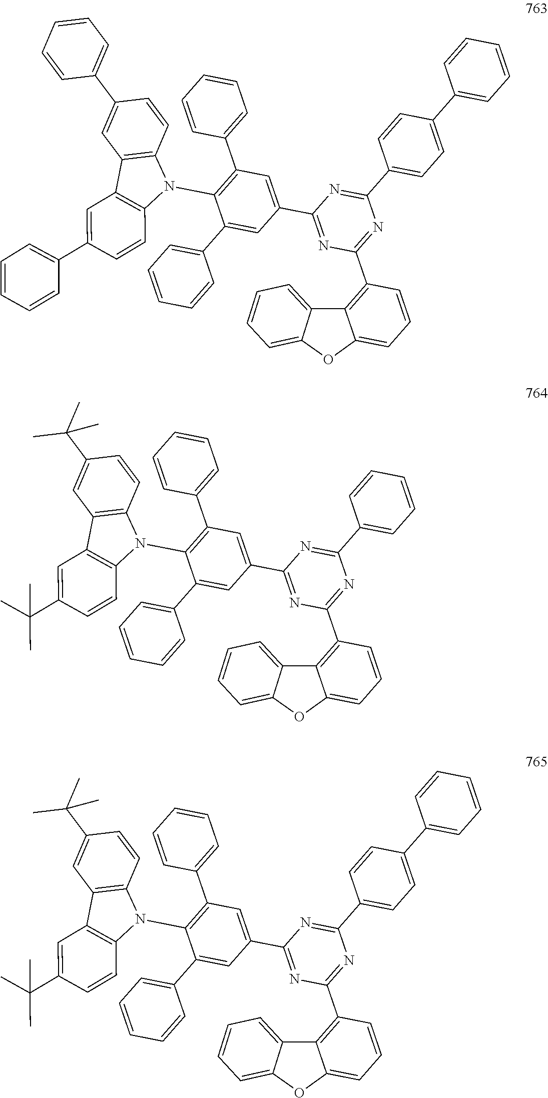 [SMILES: c1ccc(-c2ccc(-c3nc(-c4cc(-c5ccccc5)c(-n5c6ccc(-c7ccccc7)cc6c6cc(-c7ccccc7)ccc65)c(-c5ccccc5)c4)nc(-c4cccc5oc6ccccc6c45)n3)cc2)cc1 ; CC(C)(C)c1ccc2c(c1)c1cc(C(C)(C)C)ccc1n2-c1c(-c2ccccc2)cc(-c2nc(-c3ccccc3)nc(-c3cccc4oc5ccccc5c34)n2)cc1-c1ccccc1 ; CC(C)(C)c1ccc2c(c1)c1cc(C(C)(C)C)ccc1n2-c1c(-c2ccccc2)cc(-c2nc(-c3ccc(-c4ccccc4)cc3)nc(-c3cccc4oc5ccccc5c34)n2)cc1-c1ccccc1]

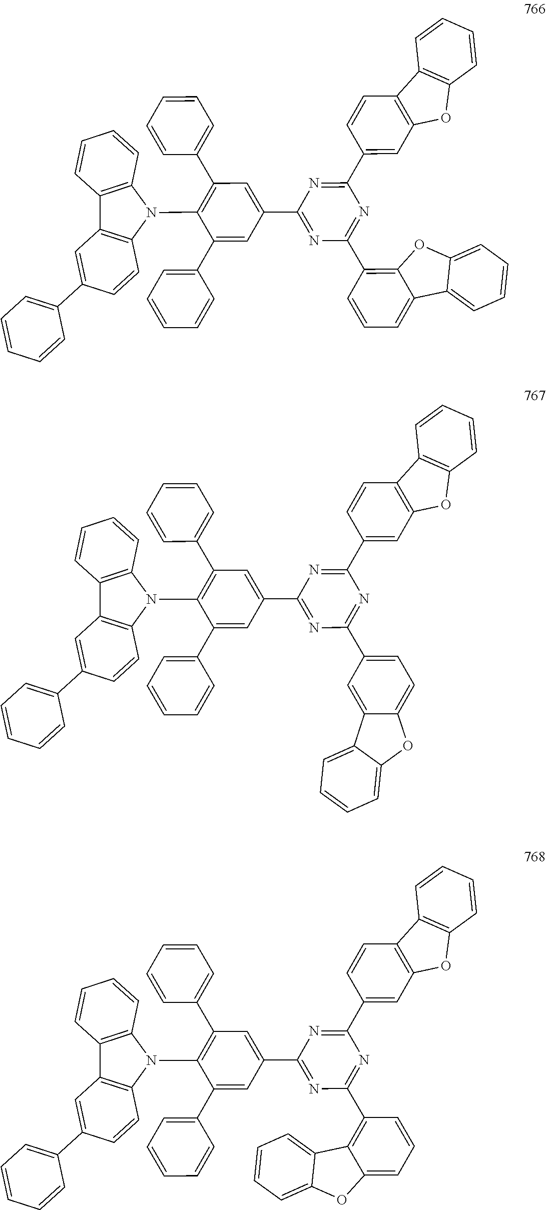 [SMILES: c1ccc(-c2ccc3c(c2)c2ccccc2n3-c2c(-c3ccccc3)cc(-c3nc(-c4ccc5c(c4)oc4ccccc45)nc(-c4cccc5c4oc4ccccc45)n3)cc2-c2ccccc2)cc1 ; c1ccc(-c2ccc3c(c2)c2ccccc2n3-c2c(-c3ccccc3)cc(-c3nc(-c4ccc5c(c4)oc4ccccc45)nc(-c4ccc5oc6ccccc6c5c4)n3)cc2-c2ccccc2)cc1 ; c1ccc(-c2ccc3c(c2)c2ccccc2n3-c2c(-c3ccccc3)cc(-c3nc(-c4ccc5c(c4)oc4ccccc45)nc(-c4cccc5oc6ccccc6c45)n3)cc2-c2ccccc2)cc1]

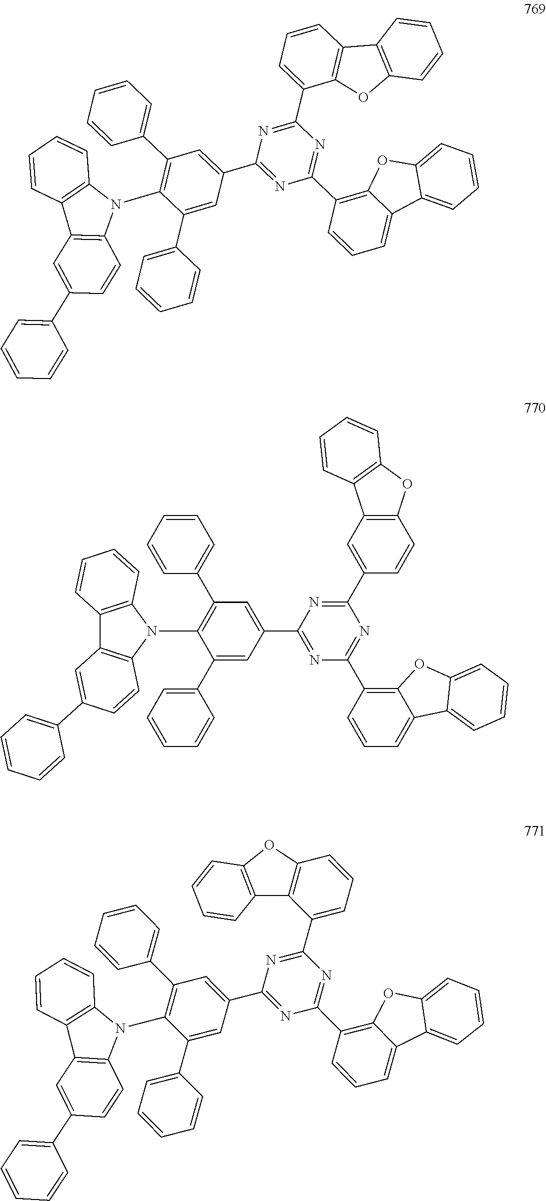 [SMILES: c1ccc(-c2ccc3c(c2)c2ccccc2n3-c2c(-c3ccccc3)cc(-c3nc(-c4cccc5c4oc4ccccc45)nc(-c4cccc5c4oc4ccccc45)n3)cc2-c2ccccc2)cc1 ; c1ccc(-c2ccc3c(c2)c2ccccc2n3-c2c(-c3ccccc3)cc(-c3nc(-c4ccc5oc6ccccc6c5c4)nc(-c4cccc5c4oc4ccccc45)n3)cc2-c2ccccc2)cc1 ; c1ccc(-c2ccc3c(c2)c2ccccc2n3-c2c(-c3ccccc3)cc(-c3nc(-c4cccc5c4oc4ccccc45)nc(-c4cccc5oc6ccccc6c45)n3)cc2-c2ccccc2)cc1]

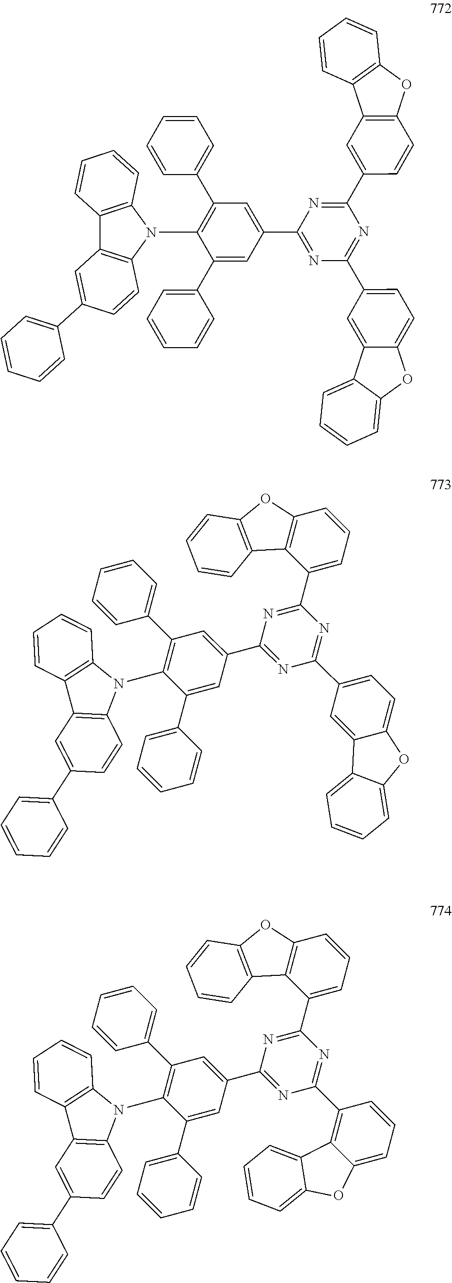 [SMILES: c1ccc(-c2ccc3c(c2)c2ccccc2n3-c2c(-c3ccccc3)cc(-c3nc(-c4ccc5oc6ccccc6c5c4)nc(-c4ccc5oc6ccccc6c5c4)n3)cc2-c2ccccc2)cc1 ; c1ccc(-c2ccc3c(c2)c2ccccc2n3-c2c(-c3ccccc3)cc(-c3nc(-c4ccc5oc6ccccc6c5c4)nc(-c4cccc5oc6ccccc6c45)n3)cc2-c2ccccc2)cc1 ; c1ccc(-c2ccc3c(c2)c2ccccc2n3-c2c(-c3ccccc3)cc(-c3nc(-c4cccc5oc6ccccc6c45)nc(-c4cccc5oc6ccccc6c45)n3)cc2-c2ccccc2)cc1]

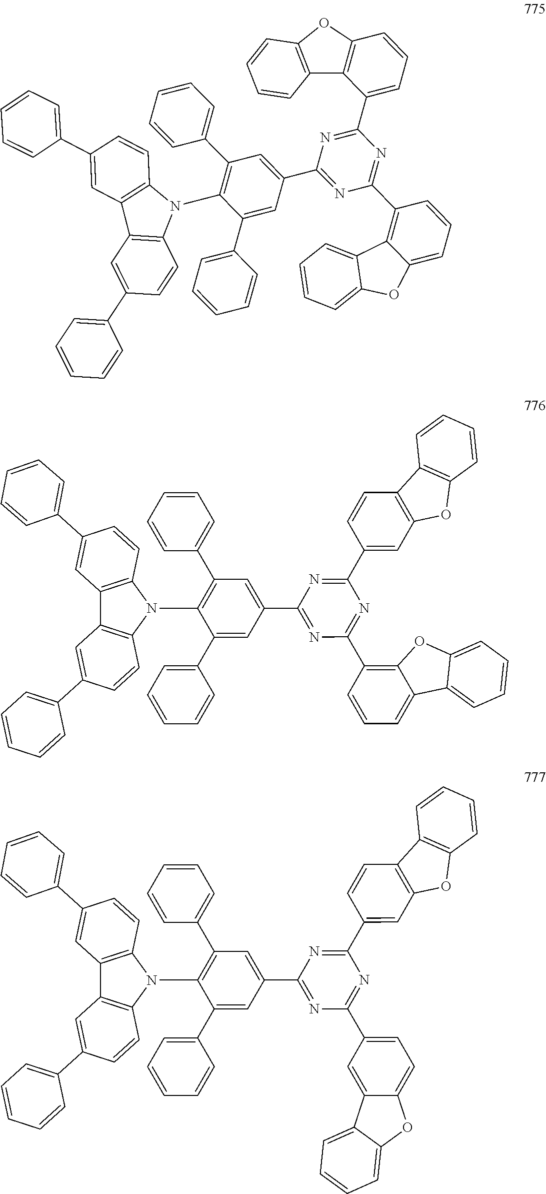 [SMILES: c1ccc(-c2ccc3c(c2)c2cc(-c4ccccc4)ccc2n3-c2c(-c3ccccc3)cc(-c3nc(-c4cccc5oc6ccccc6c45)nc(-c4cccc5oc6ccccc6c45)n3)cc2-c2ccccc2)cc1 ; c1ccc(-c2ccc3c(c2)c2cc(-c4ccccc4)ccc2n3-c2c(-c3ccccc3)cc(-c3nc(-c4ccc5c(c4)oc4ccccc45)nc(-c4cccc5c4oc4ccccc45)n3)cc2-c2ccccc2)cc1 ; c1ccc(-c2ccc3c(c2)c2cc(-c4ccccc4)ccc2n3-c2c(-c3ccccc3)cc(-c3nc(-c4ccc5c(c4)oc4ccccc45)nc(-c4ccc5oc6ccccc6c5c4)n3)cc2-c2ccccc2)cc1]

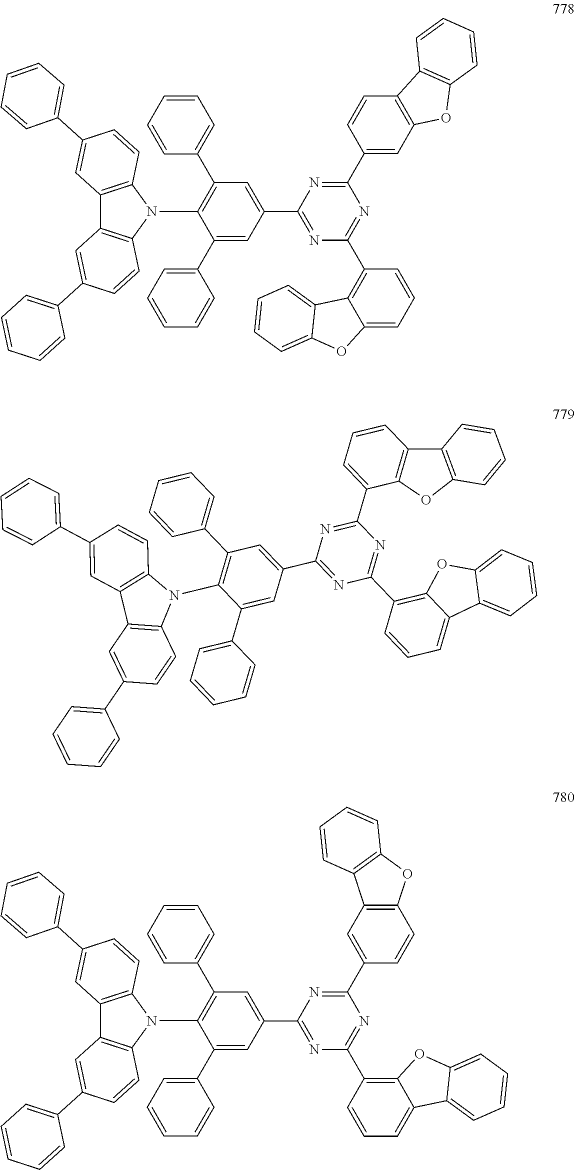 [SMILES: c1ccc(-c2ccc3c(c2)c2cc(-c4ccccc4)ccc2n3-c2c(-c3ccccc3)cc(-c3nc(-c4ccc5c(c4)oc4ccccc45)nc(-c4cccc5oc6ccccc6c45)n3)cc2-c2ccccc2)cc1 ; c1ccc(-c2ccc3c(c2)c2cc(-c4ccccc4)ccc2n3-c2c(-c3ccccc3)cc(-c3nc(-c4cccc5c4oc4ccccc45)nc(-c4cccc5c4oc4ccccc45)n3)cc2-c2ccccc2)cc1 ; c1ccc(-c2ccc3c(c2)c2cc(-c4ccccc4)ccc2n3-c2c(-c3ccccc3)cc(-c3nc(-c4ccc5oc6ccccc6c5c4)nc(-c4cccc5c4oc4ccccc45)n3)cc2-c2ccccc2)cc1]

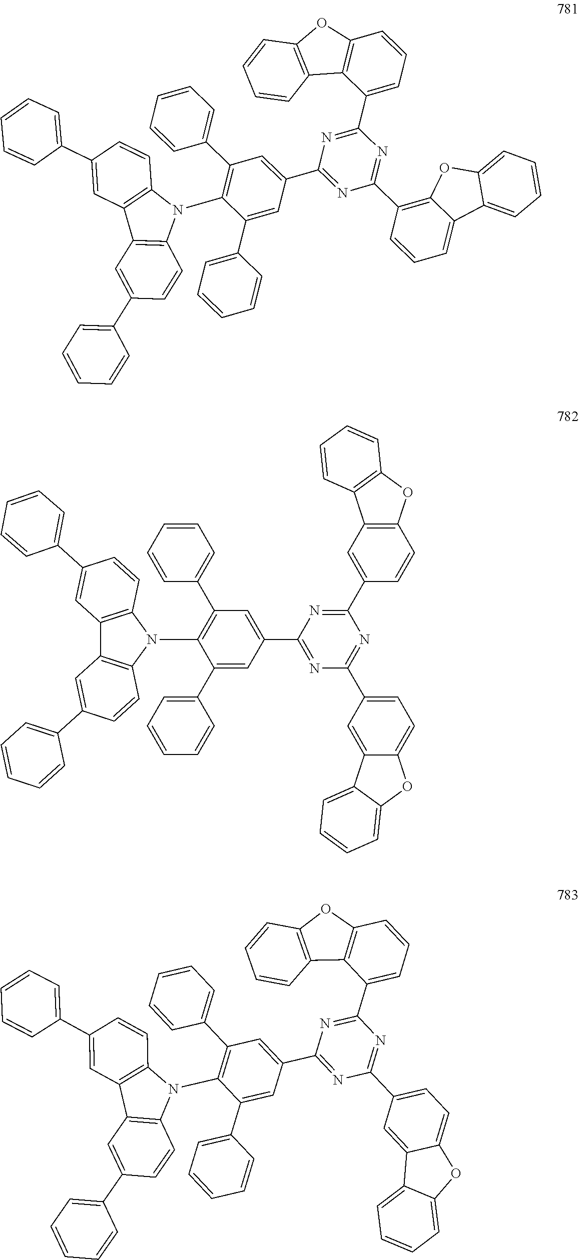 [SMILES: c1ccc(-c2ccc3c(c2)c2cc(-c4ccccc4)ccc2n3-c2c(-c3ccccc3)cc(-c3nc(-c4cccc5c4oc4ccccc45)nc(-c4cccc5oc6ccccc6c45)n3)cc2-c2ccccc2)cc1 ; c1ccc(-c2ccc3c(c2)c2cc(-c4ccccc4)ccc2n3-c2c(-c3ccccc3)cc(-c3nc(-c4ccc5oc6ccccc6c5c4)nc(-c4ccc5oc6ccccc6c5c4)n3)cc2-c2ccccc2)cc1 ; c1ccc(-c2ccc3c(c2)c2cc(-c4ccccc4)ccc2n3-c2c(-c3ccccc3)cc(-c3nc(-c4ccc5oc6ccccc6c5c4)nc(-c4cccc5oc6ccccc6c45)n3)cc2-c2ccccc2)cc1]

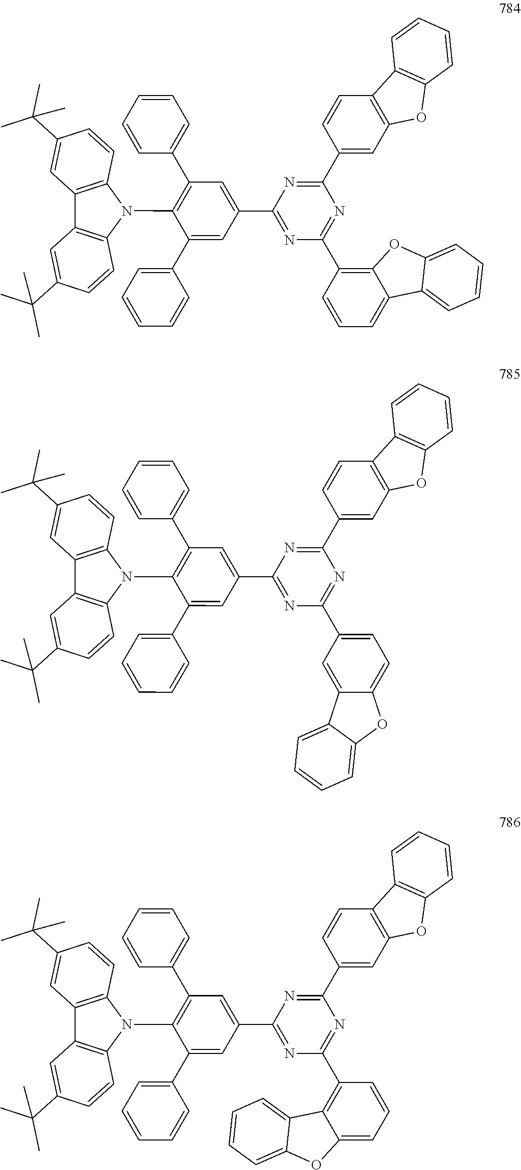 [SMILES: CC(C)(C)c1ccc2c(c1)c1cc(C(C)(C)C)ccc1n2-c1c(-c2ccccc2)cc(-c2nc(-c3ccc4c(c3)oc3ccccc34)nc(-c3cccc4c3oc3ccccc34)n2)cc1-c1ccccc1 ; CC(C)(C)c1ccc2c(c1)c1cc(C(C)(C)C)ccc1n2-c1c(-c2ccccc2)cc(-c2nc(-c3ccc4c(c3)oc3ccccc34)nc(-c3ccc4oc5ccccc5c4c3)n2)cc1-c1ccccc1 ; CC(C)(C)c1ccc2c(c1)c1cc(C(C)(C)C)ccc1n2-c1c(-c2ccccc2)cc(-c2nc(-c3ccc4c(c3)oc3ccccc34)nc(-c3cccc4oc5ccccc5c34)n2)cc1-c1ccccc1]

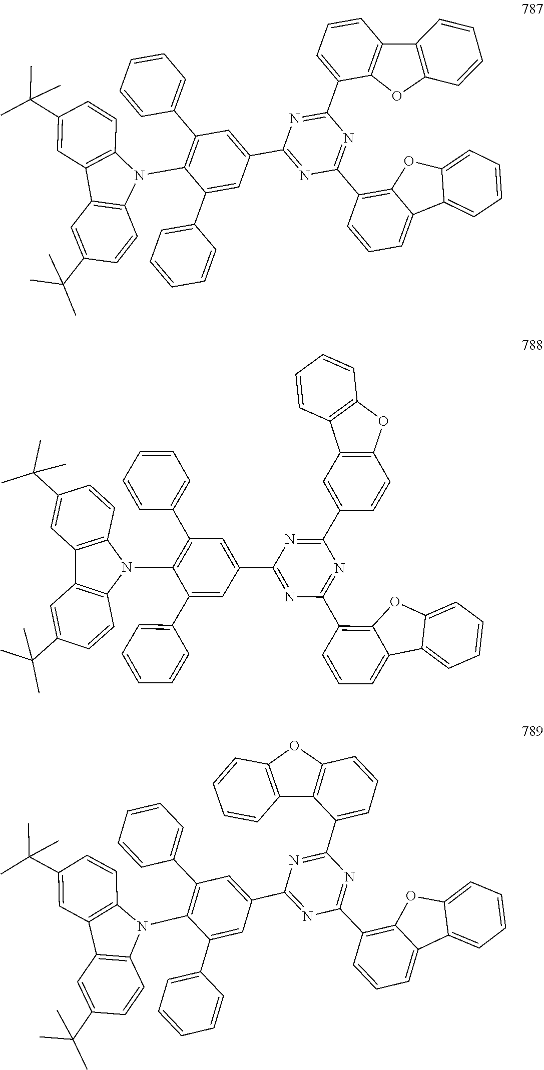 [SMILES: CC(C)(C)c1ccc2c(c1)c1cc(C(C)(C)C)ccc1n2-c1c(-c2ccccc2)cc(-c2nc(-c3cccc4c3oc3ccccc34)nc(-c3cccc4c3oc3ccccc34)n2)cc1-c1ccccc1 ; CC(C)(C)c1ccc2c(c1)c1cc(C(C)(C)C)ccc1n2-c1c(-c2ccccc2)cc(-c2nc(-c3ccc4oc5ccccc5c4c3)nc(-c3cccc4c3oc3ccccc34)n2)cc1-c1ccccc1 ; CC(C)(C)c1ccc2c(c1)c1cc(C(C)(C)C)ccc1n2-c1c(-c2ccccc2)cc(-c2nc(-c3cccc4c3oc3ccccc34)nc(-c3cccc4oc5ccccc5c34)n2)cc1-c1ccccc1]

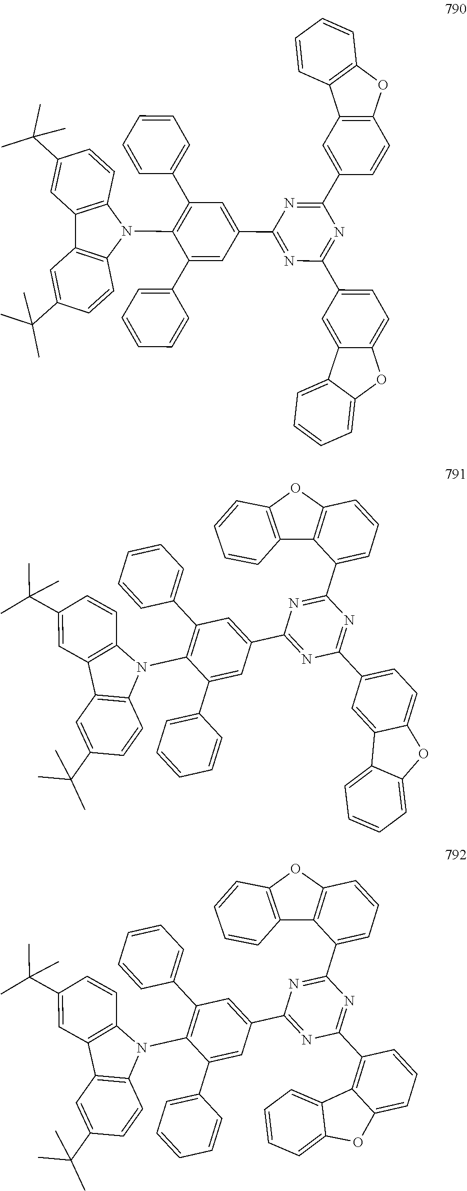 [SMILES: CC(C)(C)c1ccc2c(c1)c1cc(C(C)(C)C)ccc1n2-c1c(-c2ccccc2)cc(-c2nc(-c3ccc4oc5ccccc5c4c3)nc(-c3ccc4oc5ccccc5c4c3)n2)cc1-c1ccccc1 ; CC(C)(C)c1ccc2c(c1)c1cc(C(C)(C)C)ccc1n2-c1c(-c2ccccc2)cc(-c2nc(-c3ccc4oc5ccccc5c4c3)nc(-c3cccc4oc5ccccc5c34)n2)cc1-c1ccccc1 ; CC(C)(C)c1ccc2c(c1)c1cc(C(C)(C)C)ccc1n2-c1c(-c2ccccc2)cc(-c2nc(-c3cccc4oc5ccccc5c34)nc(-c3cccc4oc5ccccc5c34)n2)cc1-c1ccccc1]

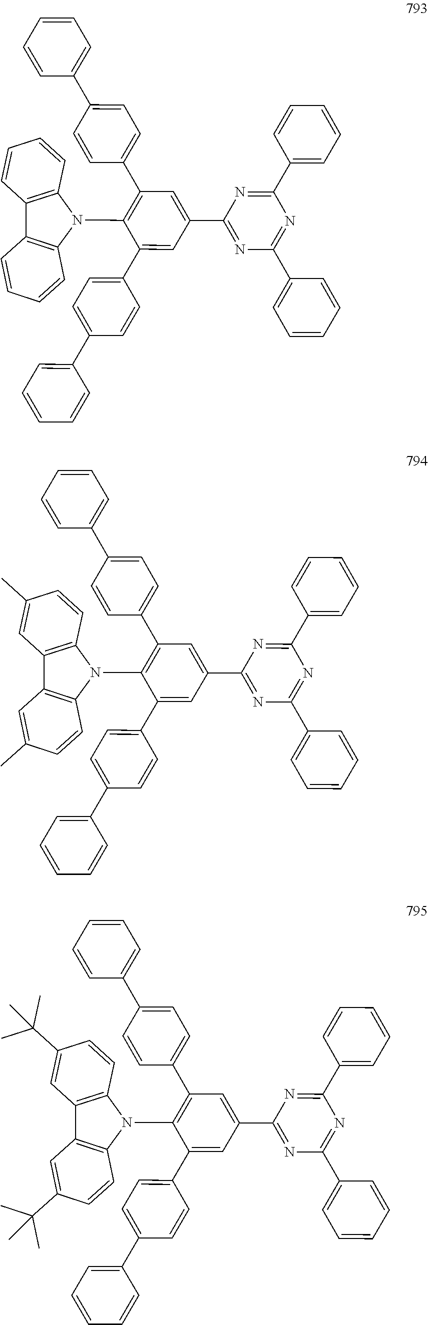

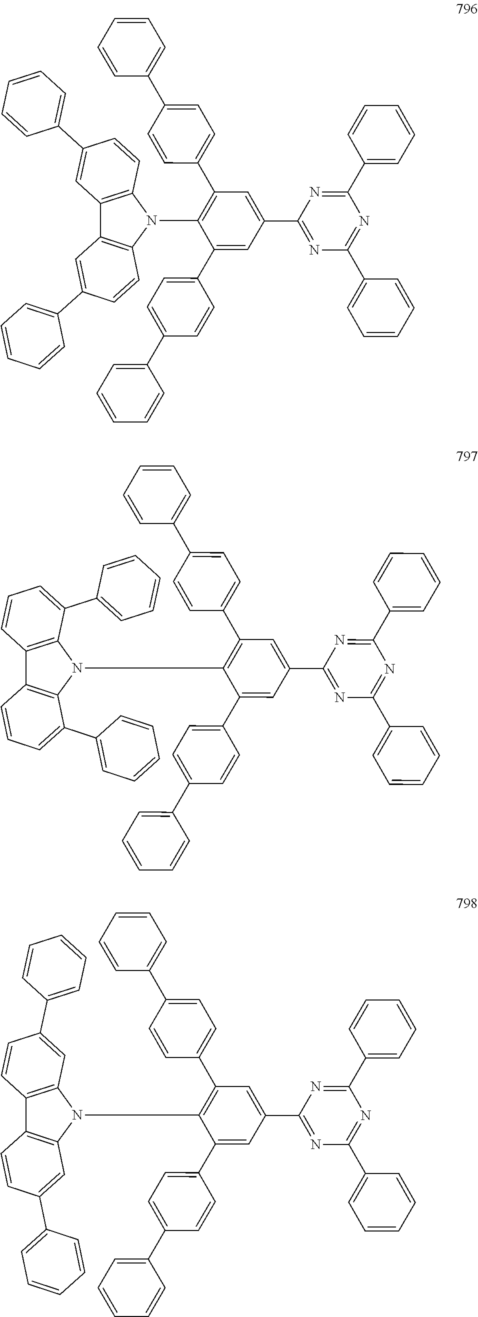

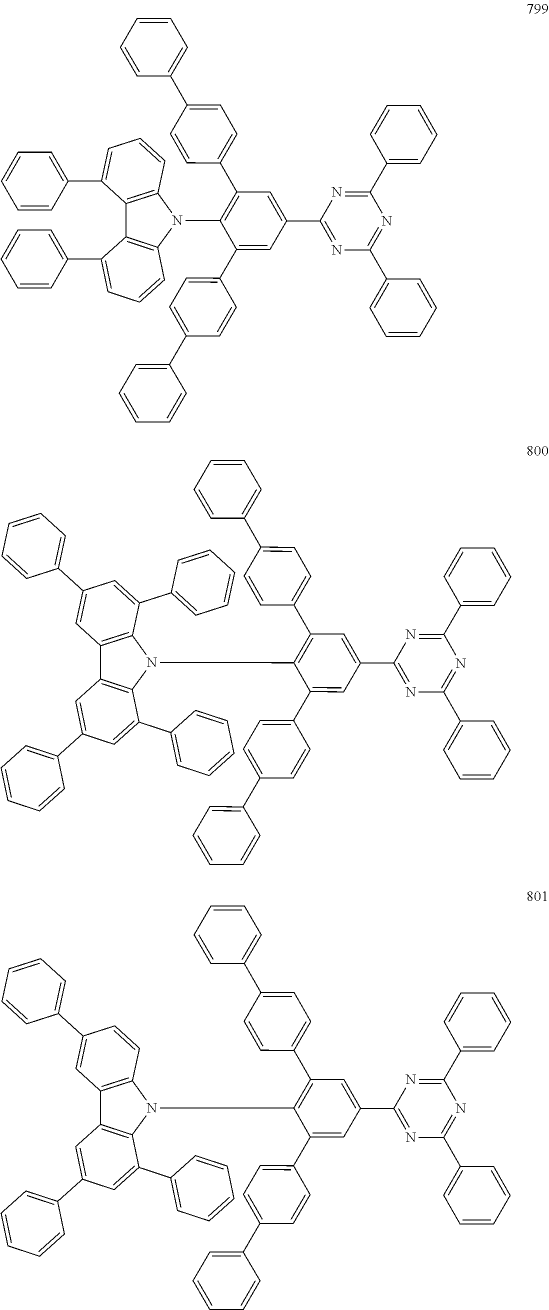

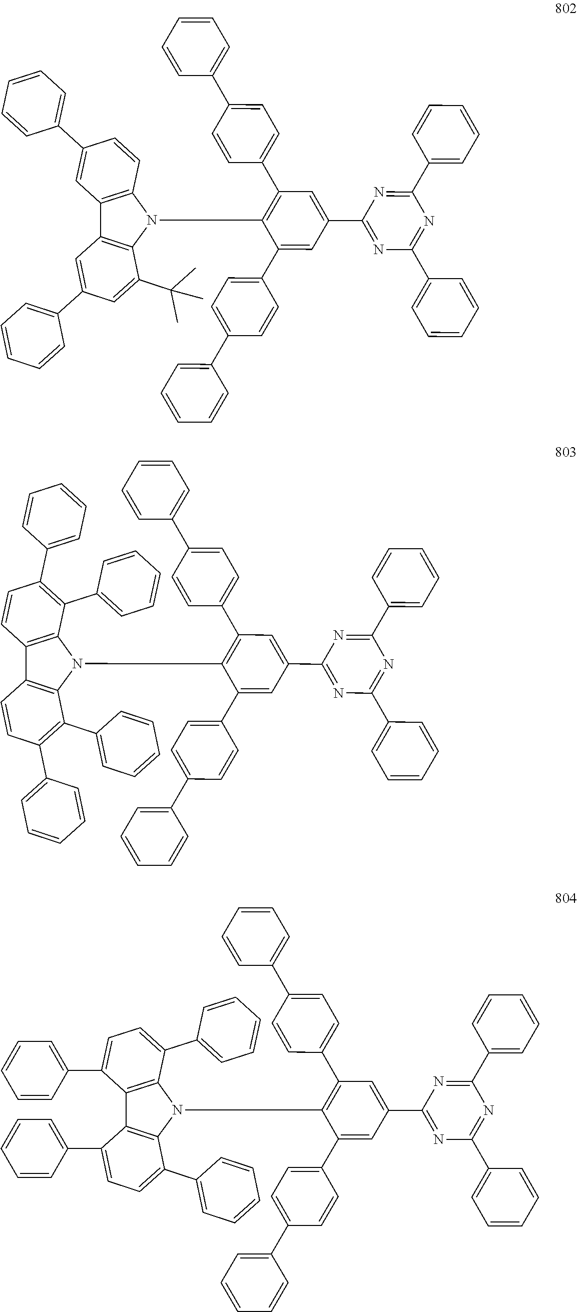

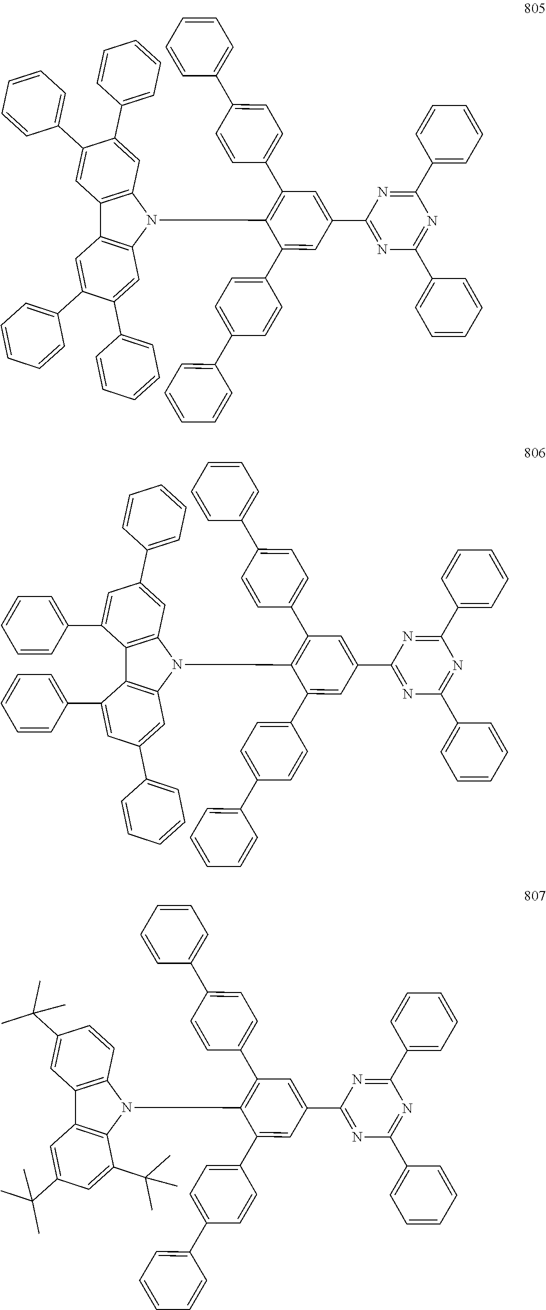

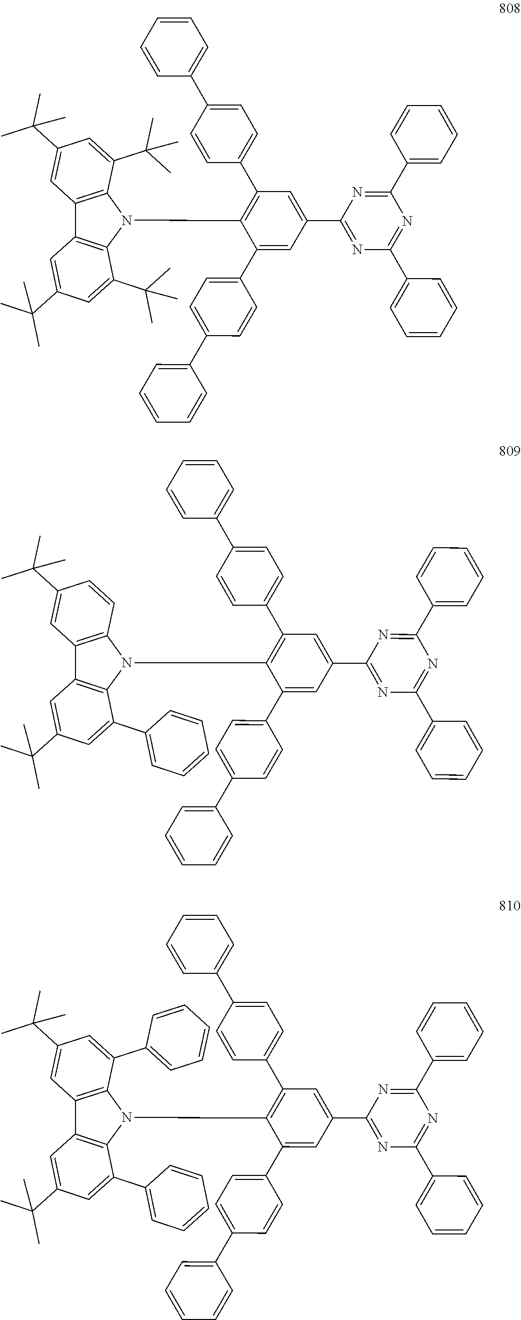

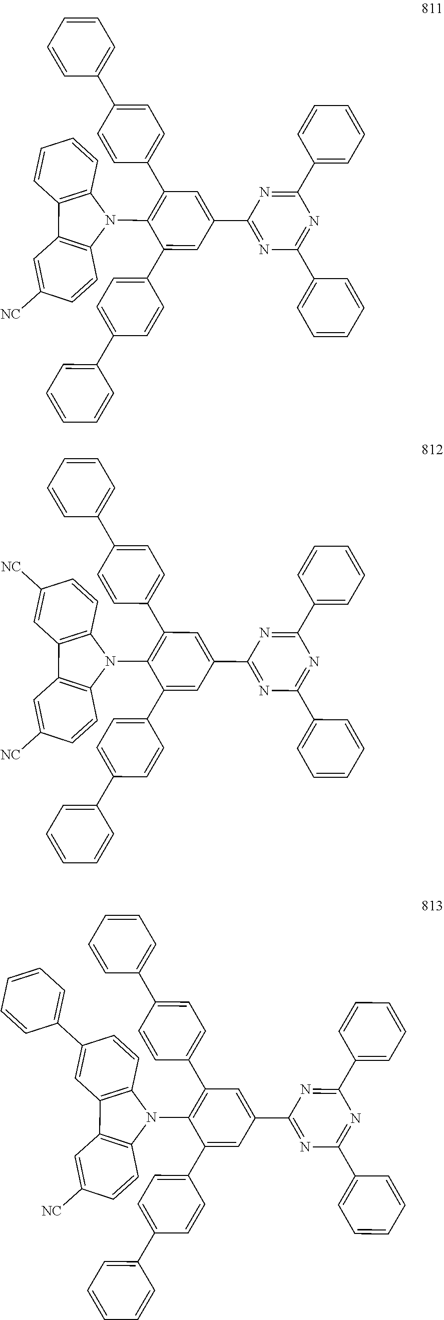

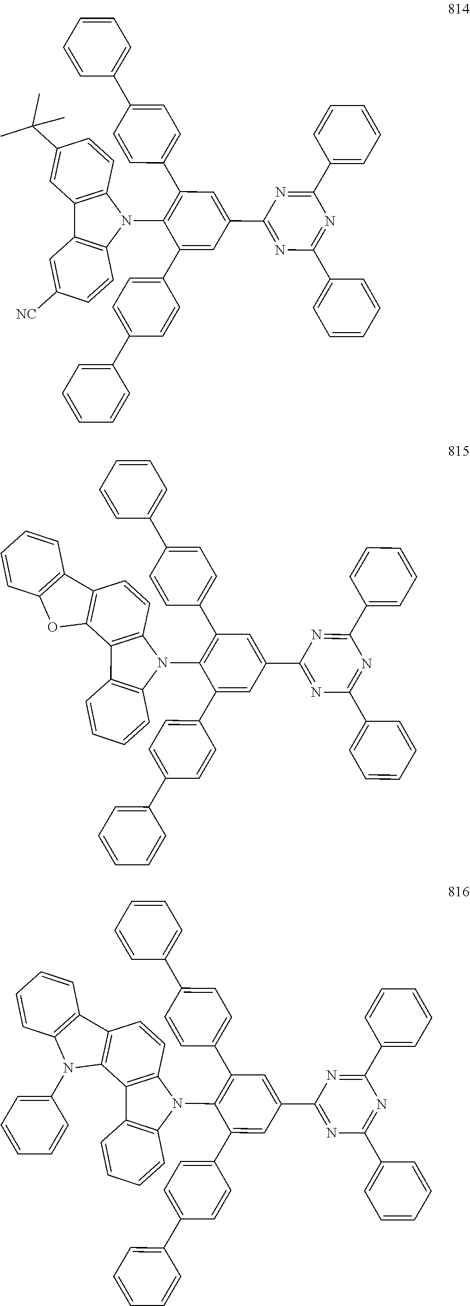

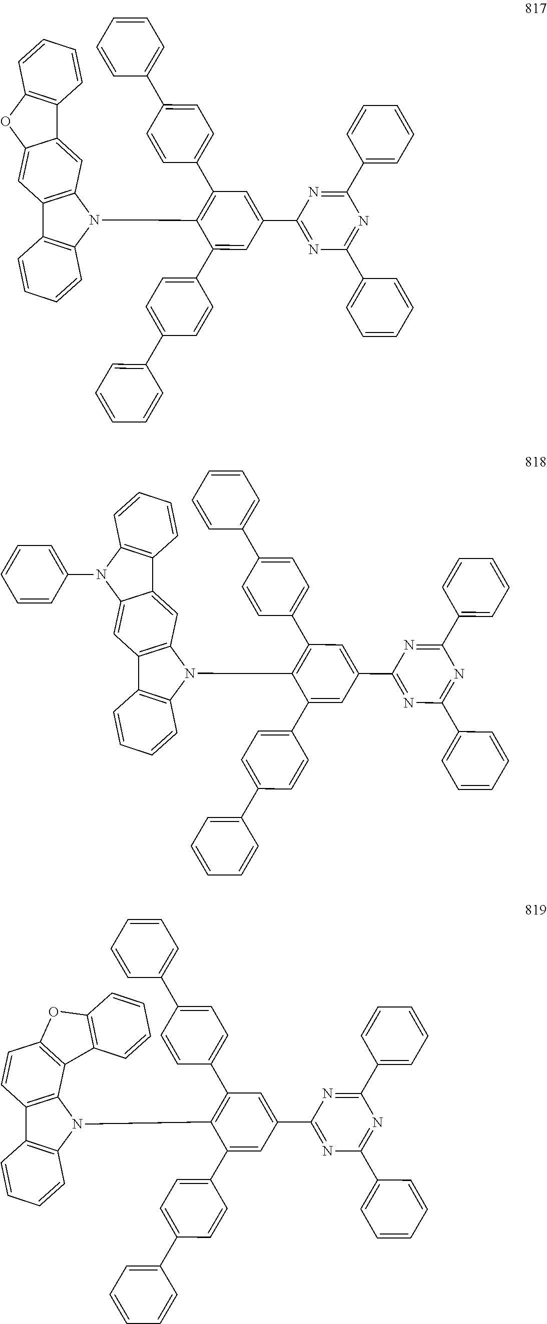 [SMILES: c1ccc(-c2ccc(-c3cc(-c4nc(-c5ccccc5)nc(-c5ccccc5)n4)cc(-c4ccc(-c5ccccc5)cc4)c3-n3c4ccccc4c4cc5oc6ccccc6c5cc43)cc2)cc1 ; c1ccc(-c2ccc(-c3cc(-c4nc(-c5ccccc5)nc(-c5ccccc5)n4)cc(-c4ccc(-c5ccccc5)cc4)c3-n3c4ccccc4c4cc5c(cc43)c3ccccc3n5-c3ccccc3)cc2)cc1 ; c1ccc(-c2ccc(-c3cc(-c4nc(-c5ccccc5)nc(-c5ccccc5)n4)cc(-c4ccc(-c5ccccc5)cc4)c3-n3c4ccccc4c4ccc5oc6ccccc6c5c43)cc2)cc1]

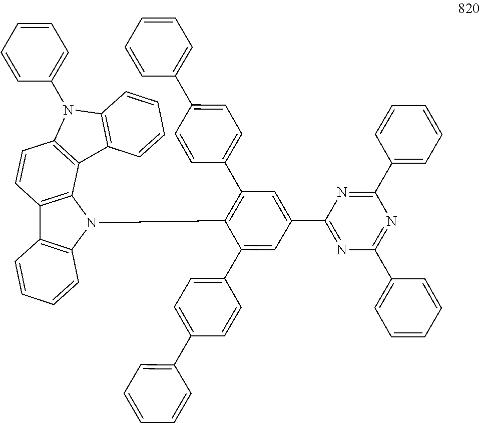 [SMILES: c1ccc(-c2ccc(-c3cc(-c4nc(-c5ccccc5)nc(-c5ccccc5)n4)cc(-c4ccc(-c5ccccc5)cc4)c3-n3c4ccccc4c4ccc5c(c6ccccc6n5-c5ccccc5)c43)cc2)cc1]

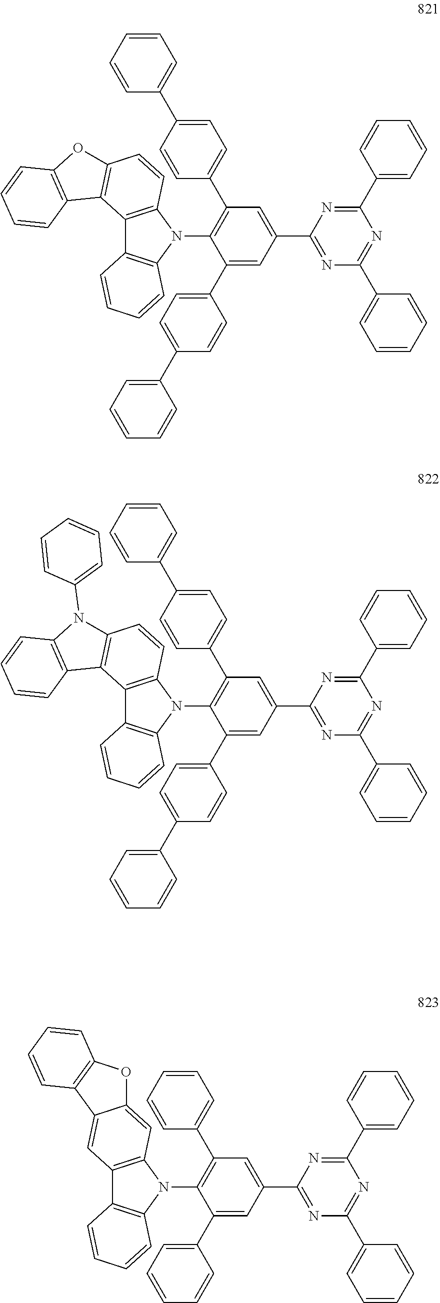 [SMILES: c1ccc(-c2ccc(-c3cc(-c4nc(-c5ccccc5)nc(-c5ccccc5)n4)cc(-c4ccc(-c5ccccc5)cc4)c3-n3c4ccccc4c4c5c(ccc43)oc3ccccc35)cc2)cc1 ; c1ccc(-c2ccc(-c3cc(-c4nc(-c5ccccc5)nc(-c5ccccc5)n4)cc(-c4ccc(-c5ccccc5)cc4)c3-n3c4ccccc4c4c5c6ccccc6n(-c6ccccc6)c5ccc43)cc2)cc1 ; c1ccc(-c2nc(-c3ccccc3)nc(-c3cc(-c4ccccc4)c(-n4c5ccccc5c5cc6c(cc54)oc4ccccc46)c(-c4ccccc4)c3)n2)cc1]

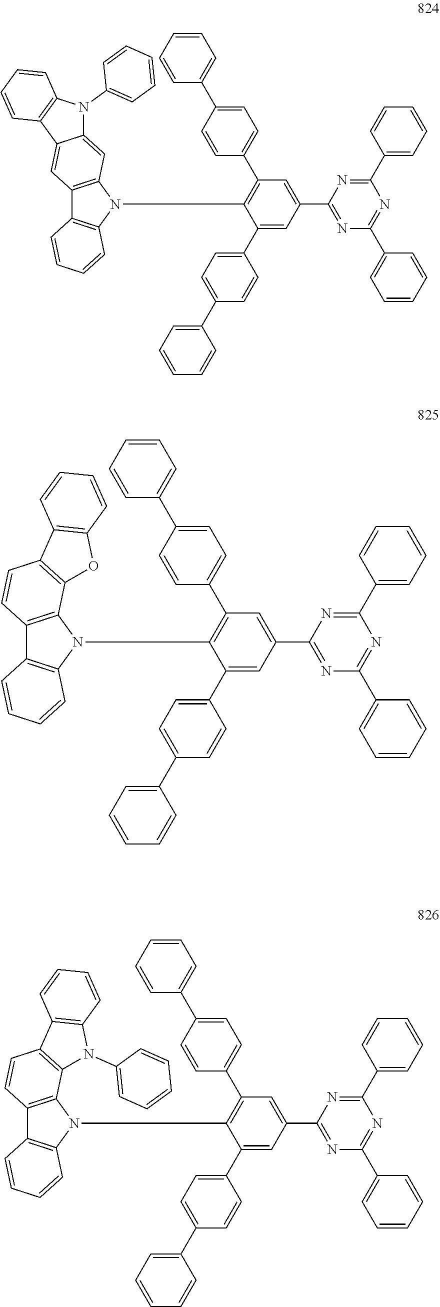 [SMILES: c1ccc(-c2ccc(-c3cc(-c4nc(-c5ccccc5)nc(-c5ccccc5)n4)cc(-c4ccc(-c5ccccc5)cc4)c3-n3c4ccccc4c4cc5c6ccccc6n(-c6ccccc6)c5cc43)cc2)cc1 ; c1ccc(-c2ccc(-c3cc(-c4nc(-c5ccccc5)nc(-c5ccccc5)n4)cc(-c4ccc(-c5ccccc5)cc4)c3-n3c4ccccc4c4ccc5c6ccccc6oc5c43)cc2)cc1 ; c1ccc(-c2ccc(-c3cc(-c4nc(-c5ccccc5)nc(-c5ccccc5)n4)cc(-c4ccc(-c5ccccc5)cc4)c3-n3c4ccccc4c4ccc5c6ccccc6n(-c6ccccc6)c5c43)cc2)cc1]

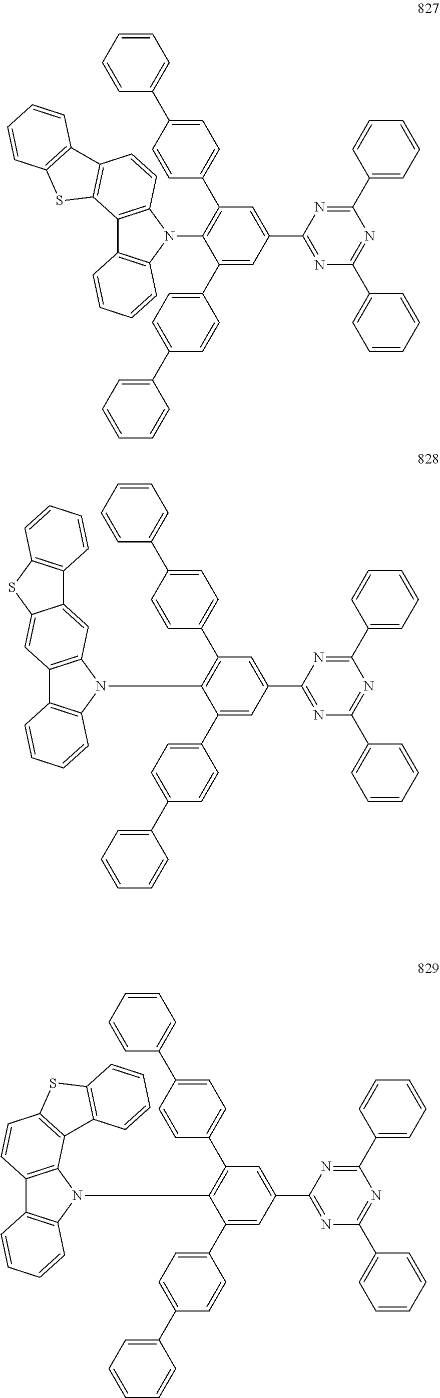

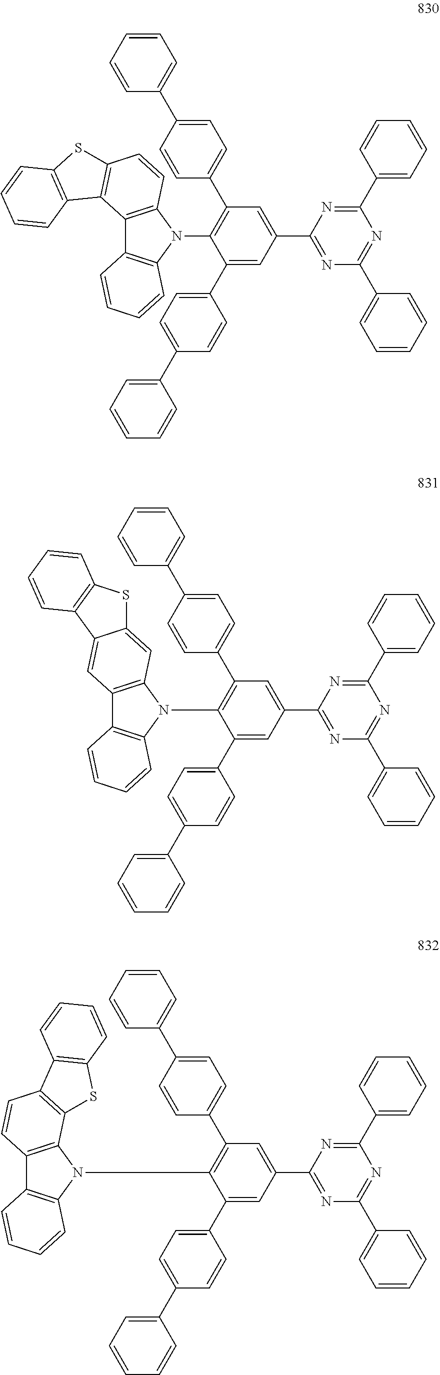 [SMILES: c1ccc(-c2ccc(-c3cc(-c4nc(-c5ccccc5)nc(-c5ccccc5)n4)cc(-c4ccc(-c5ccccc5)cc4)c3-n3c4ccccc4c4c5c(ccc43)sc3ccccc35)cc2)cc1 ; c1ccc(-c2ccc(-c3cc(-c4nc(-c5ccccc5)nc(-c5ccccc5)n4)cc(-c4ccc(-c5ccccc5)cc4)c3-n3c4ccccc4c4cc5c(cc43)sc3ccccc35)cc2)cc1 ; c1ccc(-c2ccc(-c3cc(-c4nc(-c5ccccc5)nc(-c5ccccc5)n4)cc(-c4ccc(-c5ccccc5)cc4)c3-n3c4ccccc4c4ccc5c6ccccc6sc5c43)cc2)cc1]

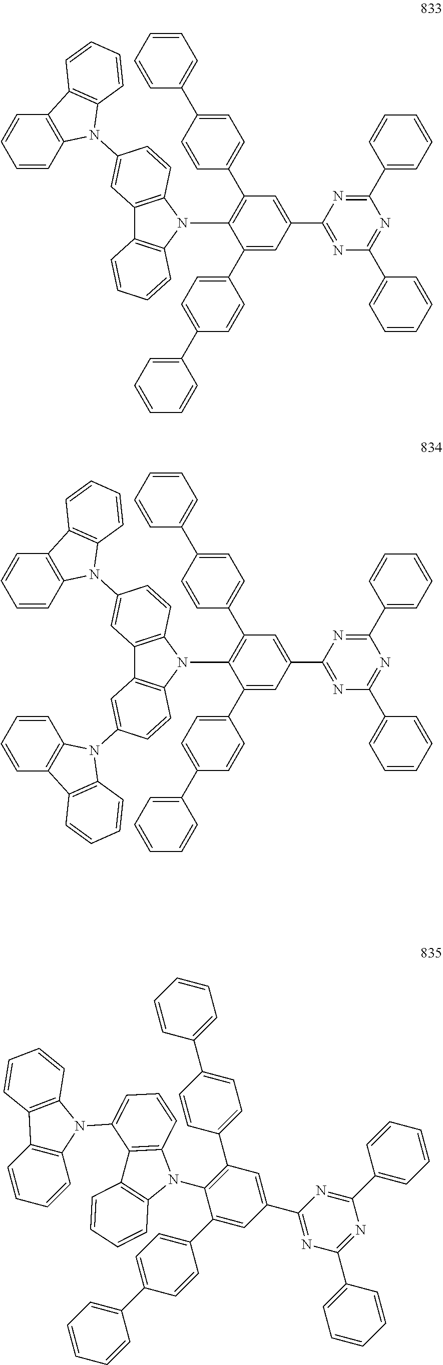 [SMILES: c1ccc(-c2ccc(-c3cc(-c4nc(-c5ccccc5)nc(-c5ccccc5)n4)cc(-c4ccc(-c5ccccc5)cc4)c3-n3c4ccccc4c4cc(-n5c6ccccc6c6ccccc65)ccc43)cc2)cc1 ; c1ccc(-c2ccc(-c3cc(-c4nc(-c5ccccc5)nc(-c5ccccc5)n4)cc(-c4ccc(-c5ccccc5)cc4)c3-n3c4ccc(-n5c6ccccc6c6ccccc65)cc4c4cc(-n5c6ccccc6c6ccccc65)ccc43)cc2)cc1 ; c1ccc(-c2ccc(-c3cc(-c4nc(-c5ccccc5)nc(-c5ccccc5)n4)cc(-c4ccc(-c5ccccc5)cc4)c3-n3c4ccccc4c4c(-n5c6ccccc6c6ccccc65)cccc43)cc2)cc1]

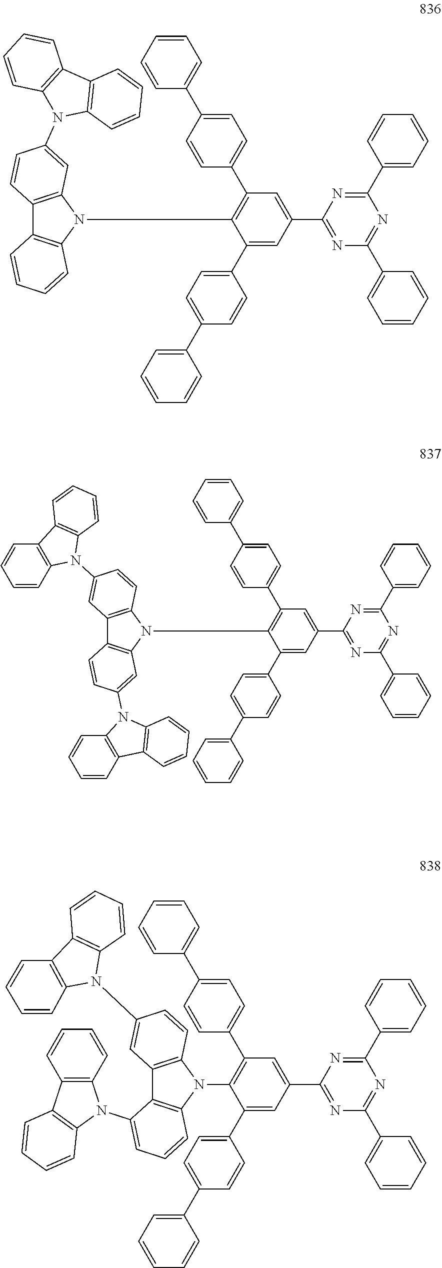 [SMILES: c1ccc(-c2ccc(-c3cc(-c4nc(-c5ccccc5)nc(-c5ccccc5)n4)cc(-c4ccc(-c5ccccc5)cc4)c3-n3c4ccccc4c4ccc(-n5c6ccccc6c6ccccc65)cc43)cc2)cc1 ; c1ccc(-c2ccc(-c3cc(-c4nc(-c5ccccc5)nc(-c5ccccc5)n4)cc(-c4ccc(-c5ccccc5)cc4)c3-n3c4ccc(-n5c6ccccc6c6ccccc65)cc4c4ccc(-n5c6ccccc6c6ccccc65)cc43)cc2)cc1 ; c1ccc(-c2ccc(-c3cc(-c4nc(-c5ccccc5)nc(-c5ccccc5)n4)cc(-c4ccc(-c5ccccc5)cc4)c3-n3c4ccc(-n5c6ccccc6c6ccccc65)cc4c4c(-n5c6ccccc6c6ccccc65)cccc43)cc2)cc1]

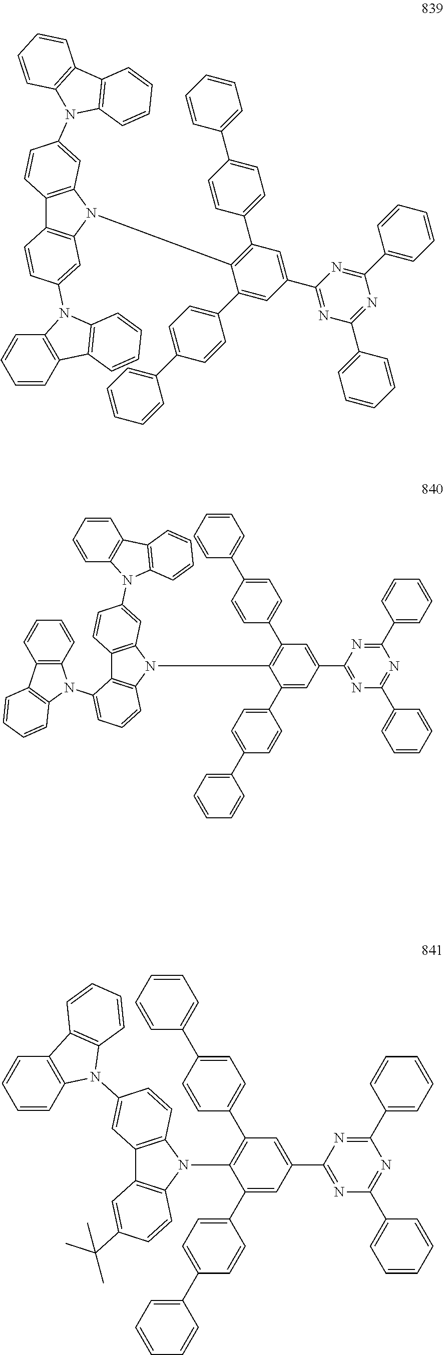 [SMILES: c1ccc(-c2ccc(-c3cc(-c4nc(-c5ccccc5)nc(-c5ccccc5)n4)cc(-c4ccc(-c5ccccc5)cc4)c3-n3c4cc(-n5c6ccccc6c6ccccc65)ccc4c4ccc(-n5c6ccccc6c6ccccc65)cc43)cc2)cc1 ; c1ccc(-c2ccc(-c3cc(-c4nc(-c5ccccc5)nc(-c5ccccc5)n4)cc(-c4ccc(-c5ccccc5)cc4)c3-n3c4cc(-n5c6ccccc6c6ccccc65)ccc4c4c(-n5c6ccccc6c6ccccc65)cccc43)cc2)cc1 ; CC(C)(C)c1ccc2c(c1)c1cc(-n3c4ccccc4c4ccccc43)ccc1n2-c1c(-c2ccc(-c3ccccc3)cc2)cc(-c2nc(-c3ccccc3)nc(-c3ccccc3)n2)cc1-c1ccc(-c2ccccc2)cc1]

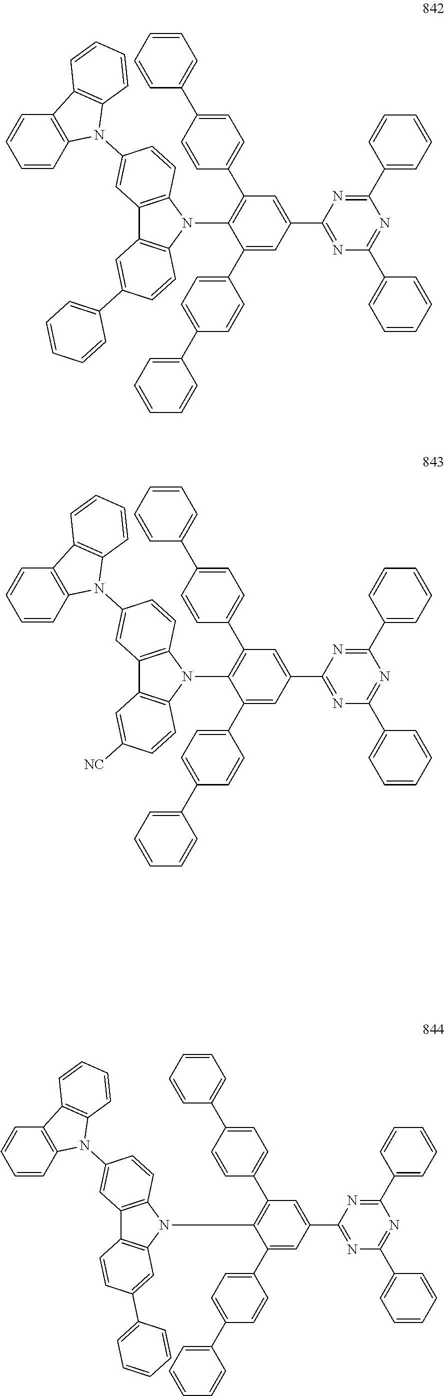 [SMILES: c1ccc(-c2ccc(-c3cc(-c4nc(-c5ccccc5)nc(-c5ccccc5)n4)cc(-c4ccc(-c5ccccc5)cc4)c3-n3c4ccc(-c5ccccc5)cc4c4cc(-n5c6ccccc6c6ccccc65)ccc43)cc2)cc1 ; N#Cc1ccc2c(c1)c1cc(-n3c4ccccc4c4ccccc43)ccc1n2-c1c(-c2ccc(-c3ccccc3)cc2)cc(-c2nc(-c3ccccc3)nc(-c3ccccc3)n2)cc1-c1ccc(-c2ccccc2)cc1 ; c1ccc(-c2ccc(-c3cc(-c4nc(-c5ccccc5)nc(-c5ccccc5)n4)cc(-c4ccc(-c5ccccc5)cc4)c3-n3c4ccc(-n5c6ccccc6c6ccccc65)cc4c4ccc(-c5ccccc5)cc43)cc2)cc1]

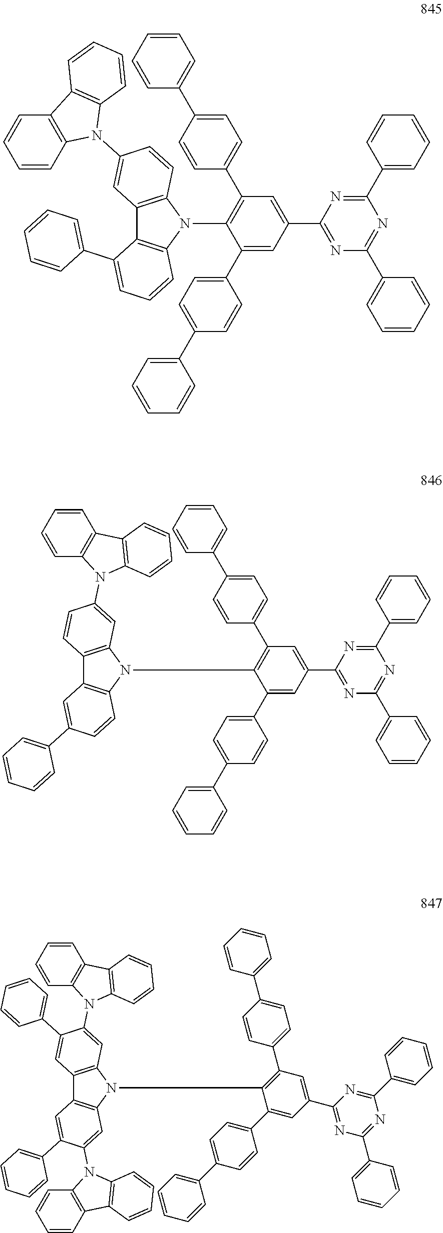 [SMILES: c1ccc(-c2ccc(-c3cc(-c4nc(-c5ccccc5)nc(-c5ccccc5)n4)cc(-c4ccc(-c5ccccc5)cc4)c3-n3c4ccc(-n5c6ccccc6c6ccccc65)cc4c4c(-c5ccccc5)cccc43)cc2)cc1 ; c1ccc(-c2ccc(-c3cc(-c4nc(-c5ccccc5)nc(-c5ccccc5)n4)cc(-c4ccc(-c5ccccc5)cc4)c3-n3c4ccc(-c5ccccc5)cc4c4ccc(-n5c6ccccc6c6ccccc65)cc43)cc2)cc1 ; c1ccc(-c2ccc(-c3cc(-c4nc(-c5ccccc5)nc(-c5ccccc5)n4)cc(-c4ccc(-c5ccccc5)cc4)c3-n3c4cc(-n5c6ccccc6c6ccccc65)c(-c5ccccc5)cc4c4cc(-c5ccccc5)c(-n5c6ccccc6c6ccccc65)cc43)cc2)cc1]

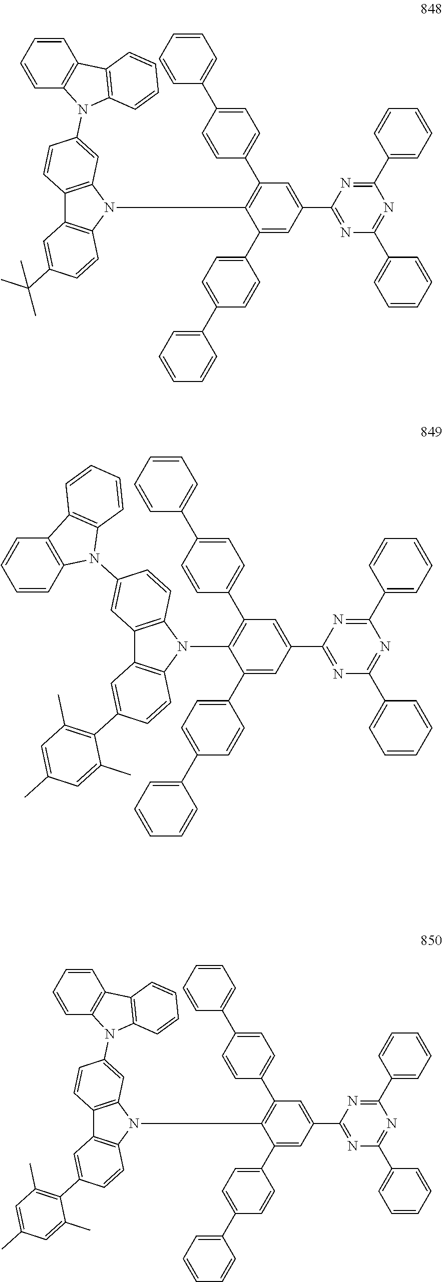 [SMILES: CC(C)(C)c1ccc2c(c1)c1ccc(-n3c4ccccc4c4ccccc43)cc1n2-c1c(-c2ccc(-c3ccccc3)cc2)cc(-c2nc(-c3ccccc3)nc(-c3ccccc3)n2)cc1-c1ccc(-c2ccccc2)cc1 ; Cc1cc(C)c(-c2ccc3c(c2)c2cc(-n4c5ccccc5c5ccccc54)ccc2n3-c2c(-c3ccc(-c4ccccc4)cc3)cc(-c3nc(-c4ccccc4)nc(-c4ccccc4)n3)cc2-c2ccc(-c3ccccc3)cc2)c(C)c1 ; Cc1cc(C)c(-c2ccc3c(c2)c2ccc(-n4c5ccccc5c5ccccc54)cc2n3-c2c(-c3ccc(-c4ccccc4)cc3)cc(-c3nc(-c4ccccc4)nc(-c4ccccc4)n3)cc2-c2ccc(-c3ccccc3)cc2)c(C)c1]

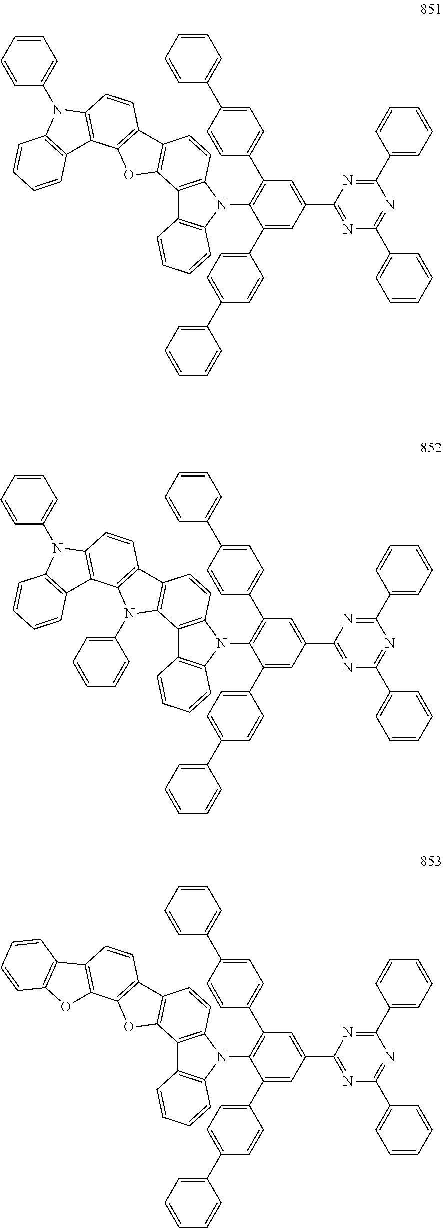 [SMILES: c1ccc(-c2ccc(-c3cc(-c4nc(-c5ccccc5)nc(-c5ccccc5)n4)cc(-c4ccc(-c5ccccc5)cc4)c3-n3c4ccccc4c4c5oc6c(ccc7c6c6ccccc6n7-c6ccccc6)c5ccc43)cc2)cc1 ; c1ccc(-c2ccc(-c3cc(-c4nc(-c5ccccc5)nc(-c5ccccc5)n4)cc(-c4ccc(-c5ccccc5)cc4)c3-n3c4ccccc4c4c3ccc3c5ccc6c(c7ccccc7n6-c6ccccc6)c5n(-c5ccccc5)c34)cc2)cc1 ; c1ccc(-c2ccc(-c3cc(-c4nc(-c5ccccc5)nc(-c5ccccc5)n4)cc(-c4ccc(-c5ccccc5)cc4)c3-n3c4ccccc4c4c5oc6c(ccc7c8ccccc8oc76)c5ccc43)cc2)cc1]

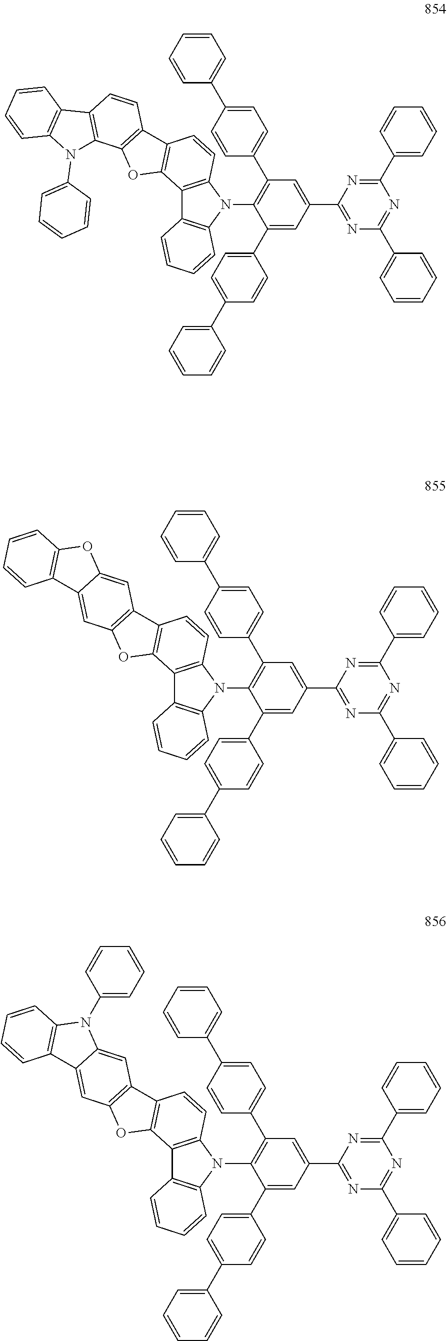 [SMILES: c1ccc(-c2ccc(-c3cc(-c4nc(-c5ccccc5)nc(-c5ccccc5)n4)cc(-c4ccc(-c5ccccc5)cc4)c3-n3c4ccccc4c4c5oc6c(ccc7c8ccccc8n(-c8ccccc8)c76)c5ccc43)cc2)cc1 ; c1ccc(-c2ccc(-c3cc(-c4nc(-c5ccccc5)nc(-c5ccccc5)n4)cc(-c4ccc(-c5ccccc5)cc4)c3-n3c4ccccc4c4c5oc6cc7c(cc6c5ccc43)oc3ccccc37)cc2)cc1 ; c1ccc(-c2ccc(-c3cc(-c4nc(-c5ccccc5)nc(-c5ccccc5)n4)cc(-c4ccc(-c5ccccc5)cc4)c3-n3c4ccccc4c4c5oc6cc7c8ccccc8n(-c8ccccc8)c7cc6c5ccc43)cc2)cc1]

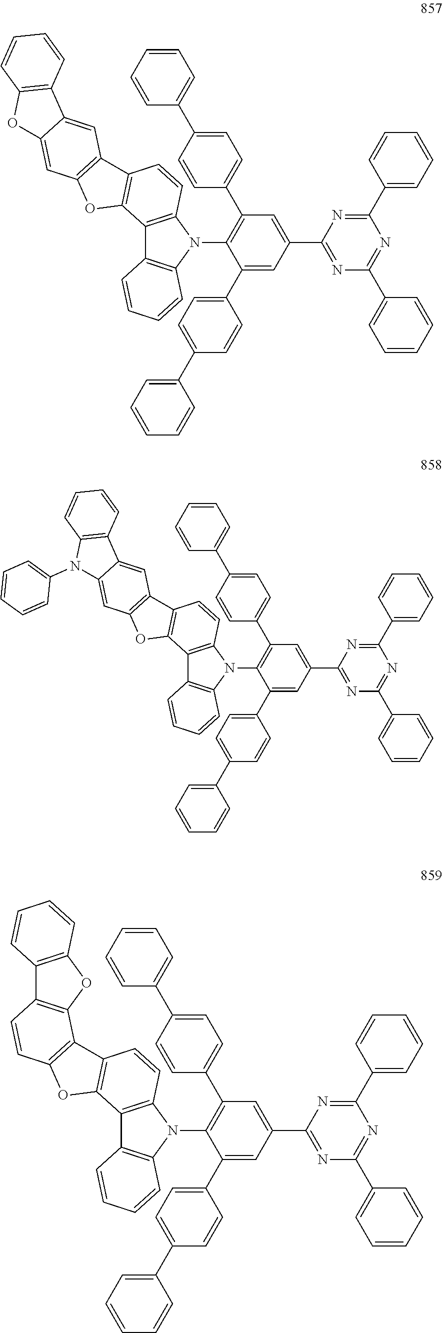 [SMILES: c1ccc(-c2ccc(-c3cc(-c4nc(-c5ccccc5)nc(-c5ccccc5)n4)cc(-c4ccc(-c5ccccc5)cc4)c3-n3c4ccccc4c4c5oc6cc7oc8ccccc8c7cc6c5ccc43)cc2)cc1 ; c1ccc(-c2ccc(-c3cc(-c4nc(-c5ccccc5)nc(-c5ccccc5)n4)cc(-c4ccc(-c5ccccc5)cc4)c3-n3c4ccccc4c4c5oc6cc7c(cc6c5ccc43)c3ccccc3n7-c3ccccc3)cc2)cc1 ; c1ccc(-c2ccc(-c3cc(-c4nc(-c5ccccc5)nc(-c5ccccc5)n4)cc(-c4ccc(-c5ccccc5)cc4)c3-n3c4ccccc4c4c5oc6ccc7c8ccccc8oc7c6c5ccc43)cc2)cc1]

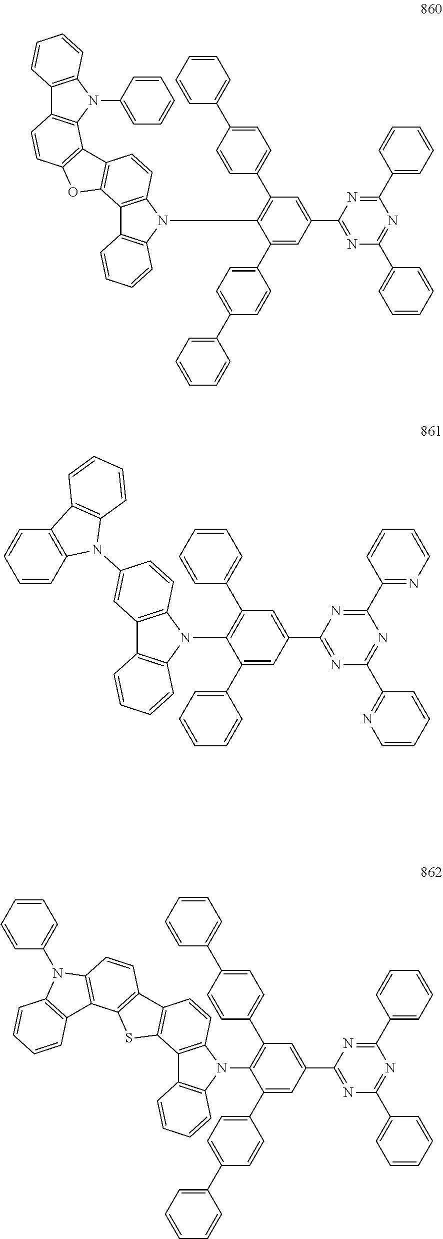 [SMILES: c1ccc(-c2ccc(-c3cc(-c4nc(-c5ccccc5)nc(-c5ccccc5)n4)cc(-c4ccc(-c5ccccc5)cc4)c3-n3c4ccccc4c4c5oc6ccc7c8ccccc8n(-c8ccccc8)c7c6c5ccc43)cc2)cc1 ; c1ccc(-c2cc(-c3nc(-c4ccccn4)nc(-c4ccccn4)n3)cc(-c3ccccc3)c2-n2c3ccccc3c3cc(-n4c5ccccc5c5ccccc54)ccc32)cc1 ; c1ccc(-c2ccc(-c3cc(-c4nc(-c5ccccc5)nc(-c5ccccc5)n4)cc(-c4ccc(-c5ccccc5)cc4)c3-n3c4ccccc4c4c5sc6c(ccc7c6c6ccccc6n7-c6ccccc6)c5ccc43)cc2)cc1]

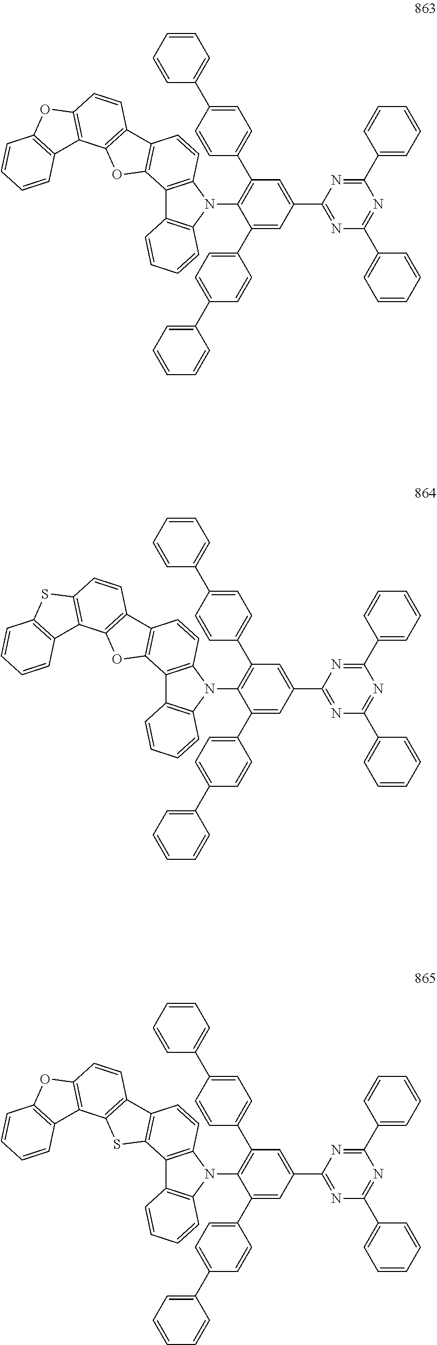 [SMILES: c1ccc(-c2ccc(-c3cc(-c4nc(-c5ccccc5)nc(-c5ccccc5)n4)cc(-c4ccc(-c5ccccc5)cc4)c3-n3c4ccccc4c4c5oc6c(ccc7oc8ccccc8c76)c5ccc43)cc2)cc1 ; c1ccc(-c2ccc(-c3cc(-c4nc(-c5ccccc5)nc(-c5ccccc5)n4)cc(-c4ccc(-c5ccccc5)cc4)c3-n3c4ccccc4c4c5oc6c(ccc7sc8ccccc8c76)c5ccc43)cc2)cc1 ; c1ccc(-c2ccc(-c3cc(-c4nc(-c5ccccc5)nc(-c5ccccc5)n4)cc(-c4ccc(-c5ccccc5)cc4)c3-n3c4ccccc4c4c5sc6c(ccc7oc8ccccc8c76)c5ccc43)cc2)cc1]

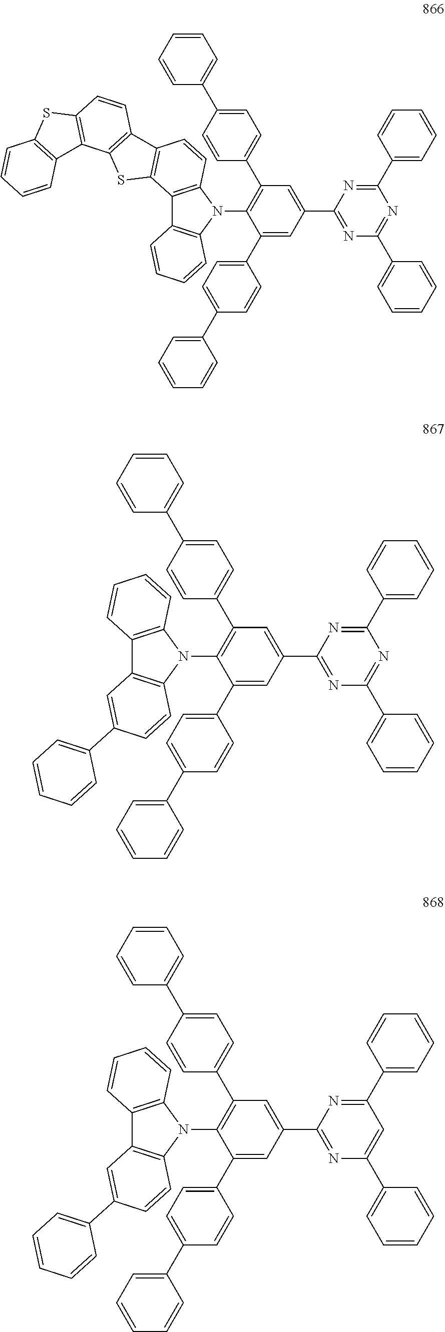 [SMILES: c1ccc(-c2ccc(-c3cc(-c4nc(-c5ccccc5)nc(-c5ccccc5)n4)cc(-c4ccc(-c5ccccc5)cc4)c3-n3c4ccccc4c4c5sc6c(ccc7sc8ccccc8c76)c5ccc43)cc2)cc1 ; c1ccc(-c2ccc(-c3cc(-c4nc(-c5ccccc5)nc(-c5ccccc5)n4)cc(-c4ccc(-c5ccccc5)cc4)c3-n3c4ccccc4c4cc(-c5ccccc5)ccc43)cc2)cc1 ; c1ccc(-c2ccc(-c3cc(-c4nc(-c5ccccc5)cc(-c5ccccc5)n4)cc(-c4ccc(-c5ccccc5)cc4)c3-n3c4ccccc4c4cc(-c5ccccc5)ccc43)cc2)cc1]

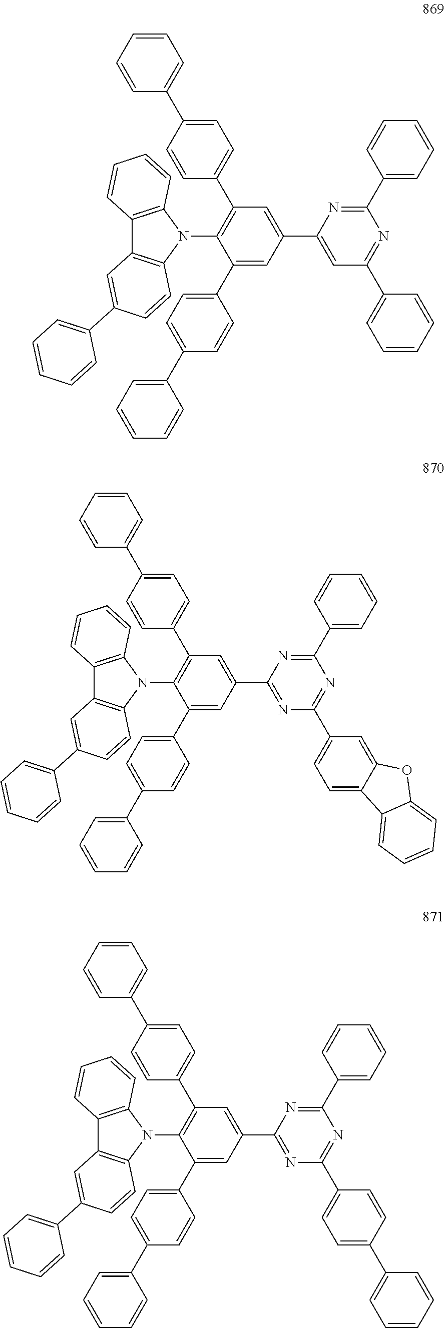 [SMILES: c1ccc(-c2ccc(-c3cc(-c4cc(-c5ccccc5)nc(-c5ccccc5)n4)cc(-c4ccc(-c5ccccc5)cc4)c3-n3c4ccccc4c4cc(-c5ccccc5)ccc43)cc2)cc1 ; c1ccc(-c2ccc(-c3cc(-c4nc(-c5ccccc5)nc(-c5ccc6c(c5)oc5ccccc56)n4)cc(-c4ccc(-c5ccccc5)cc4)c3-n3c4ccccc4c4cc(-c5ccccc5)ccc43)cc2)cc1 ; c1ccc(-c2ccc(-c3nc(-c4ccccc4)nc(-c4cc(-c5ccc(-c6ccccc6)cc5)c(-n5c6ccccc6c6cc(-c7ccccc7)ccc65)c(-c5ccc(-c6ccccc6)cc5)c4)n3)cc2)cc1]

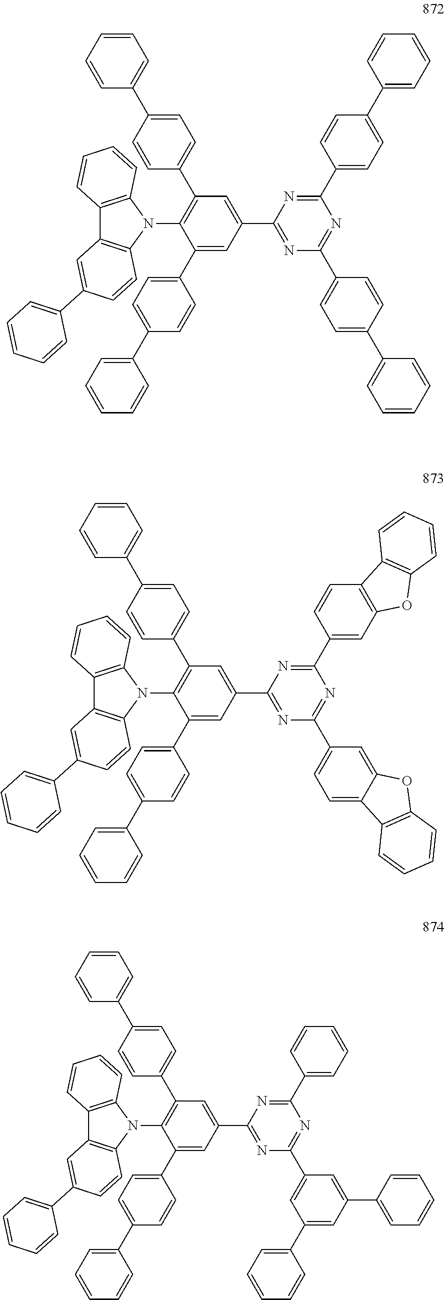 [SMILES: c1ccc(-c2ccc(-c3nc(-c4ccc(-c5ccccc5)cc4)nc(-c4cc(-c5ccc(-c6ccccc6)cc5)c(-n5c6ccccc6c6cc(-c7ccccc7)ccc65)c(-c5ccc(-c6ccccc6)cc5)c4)n3)cc2)cc1 ; c1ccc(-c2ccc(-c3cc(-c4nc(-c5ccc6c(c5)oc5ccccc56)nc(-c5ccc6c(c5)oc5ccccc56)n4)cc(-c4ccc(-c5ccccc5)cc4)c3-n3c4ccccc4c4cc(-c5ccccc5)ccc43)cc2)cc1 ; c1ccc(-c2ccc(-c3cc(-c4nc(-c5ccccc5)nc(-c5cc(-c6ccccc6)cc(-c6ccccc6)c5)n4)cc(-c4ccc(-c5ccccc5)cc4)c3-n3c4ccccc4c4cc(-c5ccccc5)ccc43)cc2)cc1]

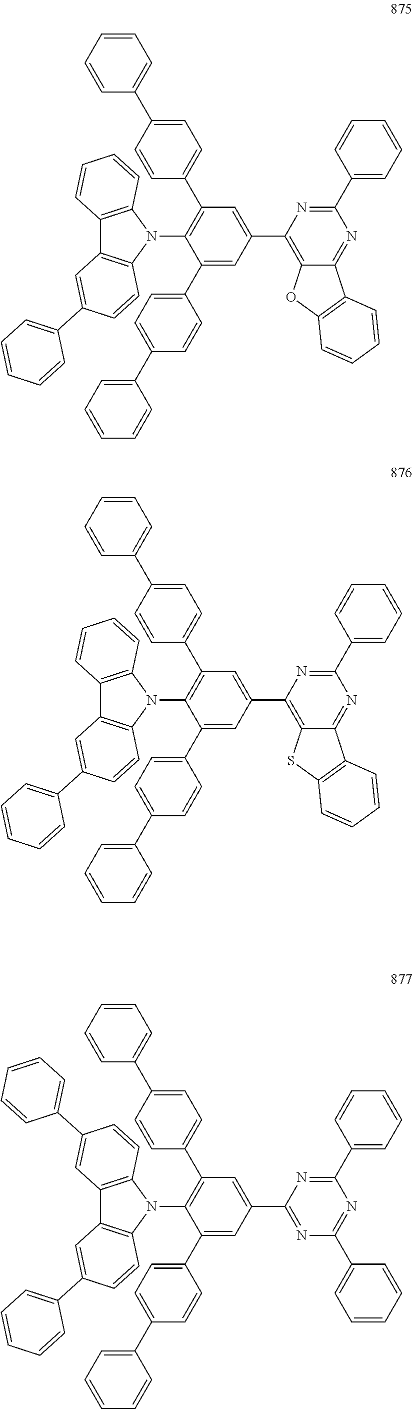 [SMILES: c1ccc(-c2ccc(-c3cc(-c4nc(-c5ccccc5)nc5c4oc4ccccc45)cc(-c4ccc(-c5ccccc5)cc4)c3-n3c4ccccc4c4cc(-c5ccccc5)ccc43)cc2)cc1 ; c1ccc(-c2ccc(-c3cc(-c4nc(-c5ccccc5)nc5c4sc4ccccc45)cc(-c4ccc(-c5ccccc5)cc4)c3-n3c4ccccc4c4cc(-c5ccccc5)ccc43)cc2)cc1 ; c1ccc(-c2ccc(-c3cc(-c4nc(-c5ccccc5)nc(-c5ccccc5)n4)cc(-c4ccc(-c5ccccc5)cc4)c3-n3c4ccc(-c5ccccc5)cc4c4cc(-c5ccccc5)ccc43)cc2)cc1]

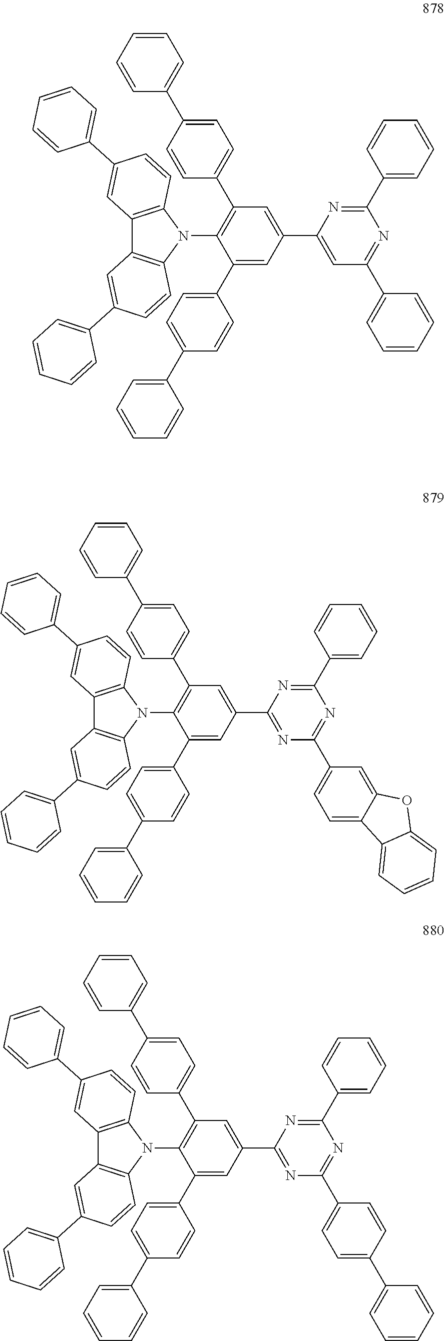 [SMILES: c1ccc(-c2ccc(-c3cc(-c4cc(-c5ccccc5)nc(-c5ccccc5)n4)cc(-c4ccc(-c5ccccc5)cc4)c3-n3c4ccc(-c5ccccc5)cc4c4cc(-c5ccccc5)ccc43)cc2)cc1 ; c1ccc(-c2ccc(-c3cc(-c4nc(-c5ccccc5)nc(-c5ccc6c(c5)oc5ccccc56)n4)cc(-c4ccc(-c5ccccc5)cc4)c3-n3c4ccc(-c5ccccc5)cc4c4cc(-c5ccccc5)ccc43)cc2)cc1 ; c1ccc(-c2ccc(-c3nc(-c4ccccc4)nc(-c4cc(-c5ccc(-c6ccccc6)cc5)c(-n5c6ccc(-c7ccccc7)cc6c6cc(-c7ccccc7)ccc65)c(-c5ccc(-c6ccccc6)cc5)c4)n3)cc2)cc1]

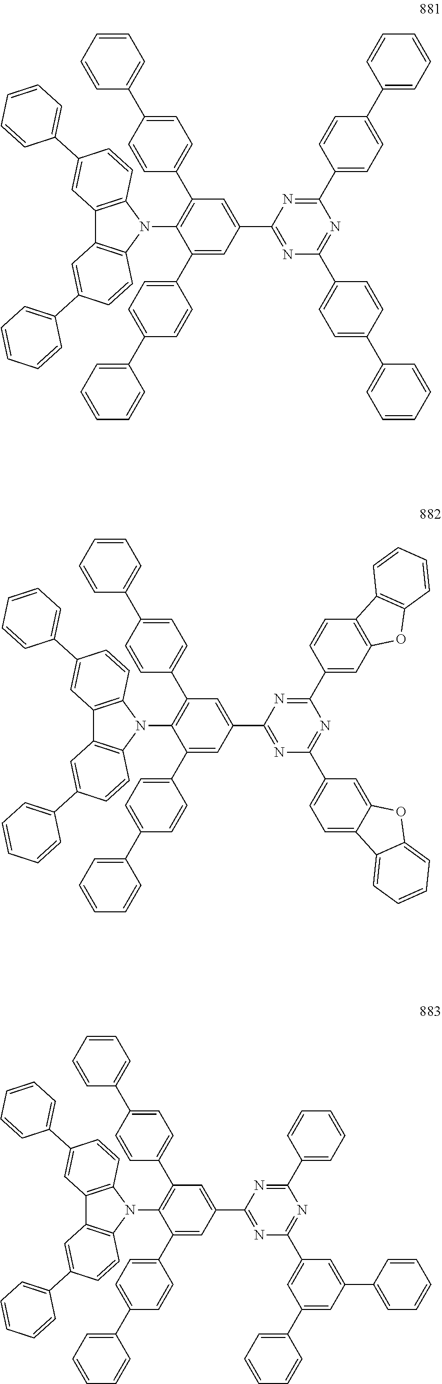 [SMILES: c1ccc(-c2ccc(-c3nc(-c4ccc(-c5ccccc5)cc4)nc(-c4cc(-c5ccc(-c6ccccc6)cc5)c(-n5c6ccc(-c7ccccc7)cc6c6cc(-c7ccccc7)ccc65)c(-c5ccc(-c6ccccc6)cc5)c4)n3)cc2)cc1 ; c1ccc(-c2ccc(-c3cc(-c4nc(-c5ccc6c(c5)oc5ccccc56)nc(-c5ccc6c(c5)oc5ccccc56)n4)cc(-c4ccc(-c5ccccc5)cc4)c3-n3c4ccc(-c5ccccc5)cc4c4cc(-c5ccccc5)ccc43)cc2)cc1 ; c1ccc(-c2ccc(-c3cc(-c4nc(-c5ccccc5)nc(-c5cc(-c6ccccc6)cc(-c6ccccc6)c5)n4)cc(-c4ccc(-c5ccccc5)cc4)c3-n3c4ccc(-c5ccccc5)cc4c4cc(-c5ccccc5)ccc43)cc2)cc1]

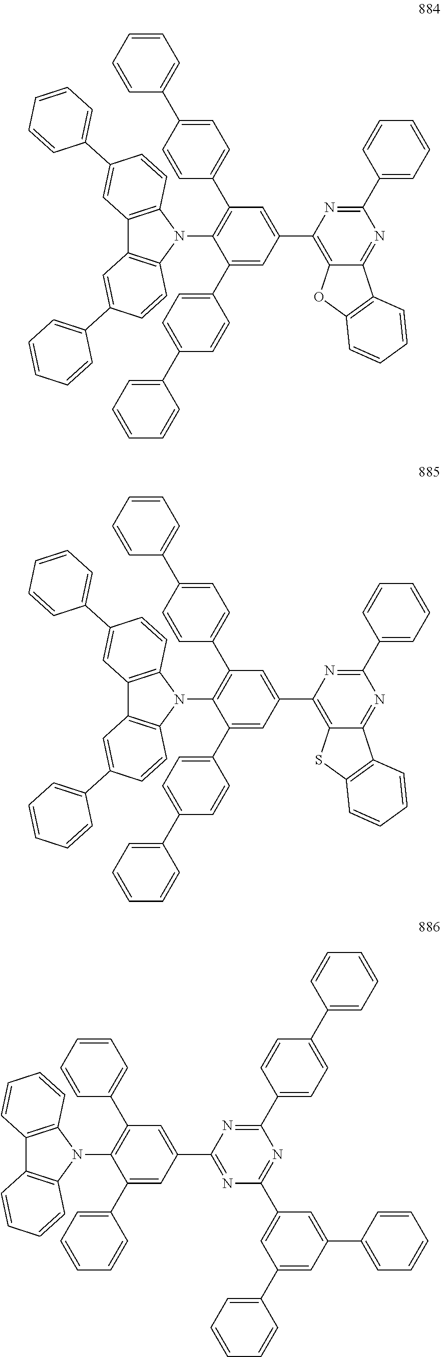 [SMILES: c1ccc(-c2ccc(-c3cc(-c4nc(-c5ccccc5)nc5c4oc4ccccc45)cc(-c4ccc(-c5ccccc5)cc4)c3-n3c4ccc(-c5ccccc5)cc4c4cc(-c5ccccc5)ccc43)cc2)cc1 ; c1ccc(-c2ccc(-c3cc(-c4nc(-c5ccccc5)nc5c4sc4ccccc45)cc(-c4ccc(-c5ccccc5)cc4)c3-n3c4ccc(-c5ccccc5)cc4c4cc(-c5ccccc5)ccc43)cc2)cc1 ; c1ccc(-c2ccc(-c3nc(-c4cc(-c5ccccc5)cc(-c5ccccc5)c4)nc(-c4cc(-c5ccccc5)c(-n5c6ccccc6c6ccccc65)c(-c5ccccc5)c4)n3)cc2)cc1]

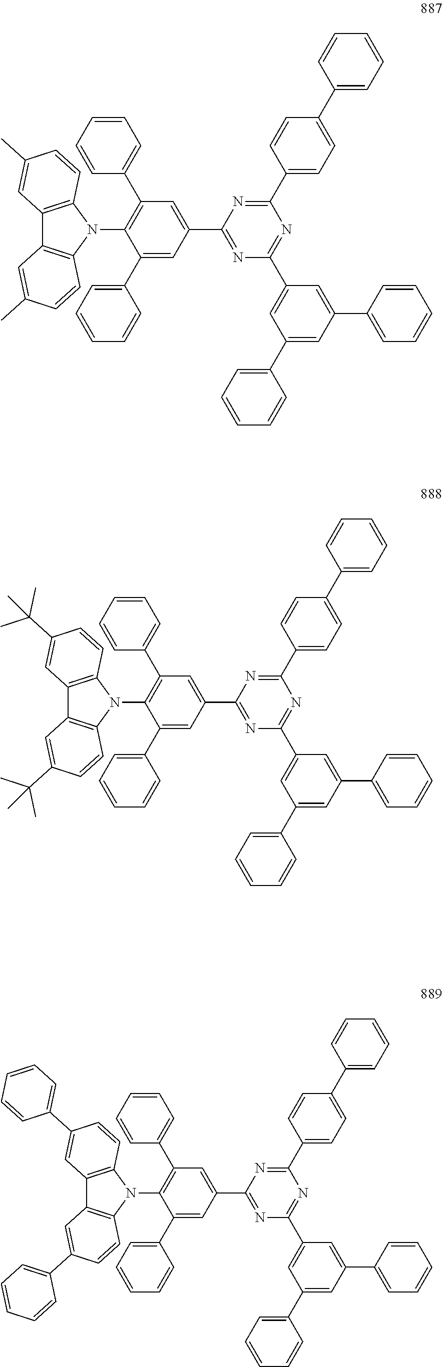 [SMILES: Cc1ccc2c(c1)c1cc(C)ccc1n2-c1c(-c2ccccc2)cc(-c2nc(-c3ccc(-c4ccccc4)cc3)nc(-c3cc(-c4ccccc4)cc(-c4ccccc4)c3)n2)cc1-c1ccccc1 ; CC(C)(C)c1ccc2c(c1)c1cc(C(C)(C)C)ccc1n2-c1c(-c2ccccc2)cc(-c2nc(-c3ccc(-c4ccccc4)cc3)nc(-c3cc(-c4ccccc4)cc(-c4ccccc4)c3)n2)cc1-c1ccccc1 ; c1ccc(-c2ccc(-c3nc(-c4cc(-c5ccccc5)cc(-c5ccccc5)c4)nc(-c4cc(-c5ccccc5)c(-n5c6ccc(-c7ccccc7)cc6c6cc(-c7ccccc7)ccc65)c(-c5ccccc5)c4)n3)cc2)cc1]

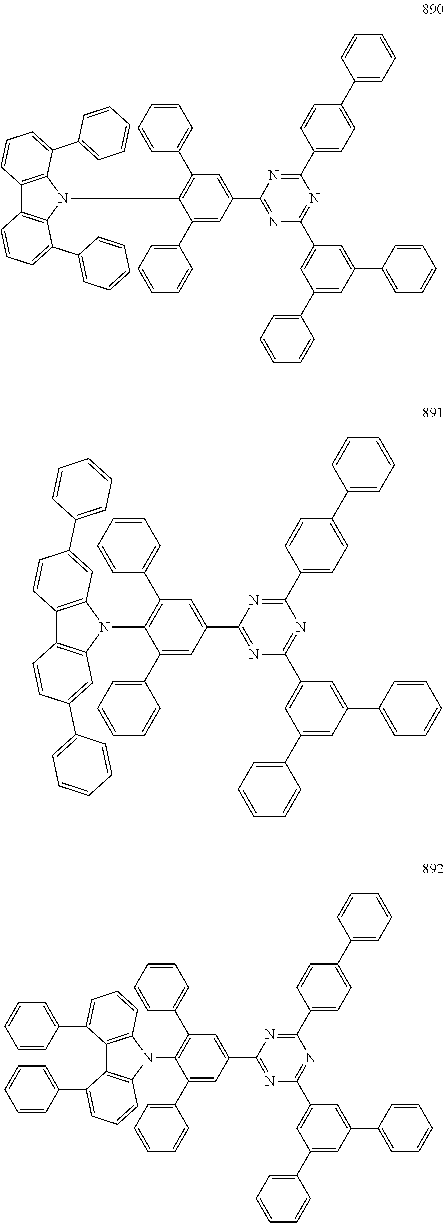 [SMILES: c1ccc(-c2ccc(-c3nc(-c4cc(-c5ccccc5)cc(-c5ccccc5)c4)nc(-c4cc(-c5ccccc5)c(-n5c6c(-c7ccccc7)cccc6c6cccc(-c7ccccc7)c65)c(-c5ccccc5)c4)n3)cc2)cc1 ; c1ccc(-c2ccc(-c3nc(-c4cc(-c5ccccc5)cc(-c5ccccc5)c4)nc(-c4cc(-c5ccccc5)c(-n5c6cc(-c7ccccc7)ccc6c6ccc(-c7ccccc7)cc65)c(-c5ccccc5)c4)n3)cc2)cc1 ; c1ccc(-c2ccc(-c3nc(-c4cc(-c5ccccc5)cc(-c5ccccc5)c4)nc(-c4cc(-c5ccccc5)c(-n5c6cccc(-c7ccccc7)c6c6c(-c7ccccc7)cccc65)c(-c5ccccc5)c4)n3)cc2)cc1]

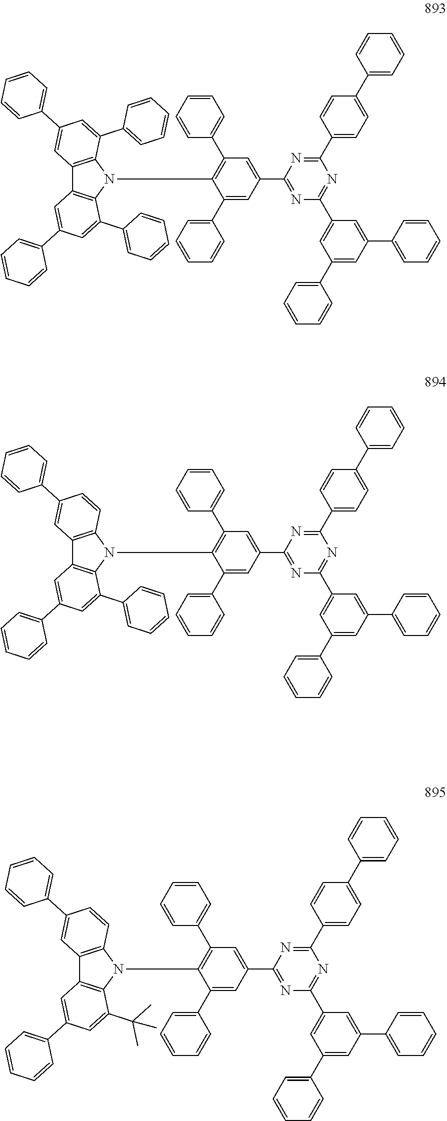 [SMILES: c1ccc(-c2ccc(-c3nc(-c4cc(-c5ccccc5)cc(-c5ccccc5)c4)nc(-c4cc(-c5ccccc5)c(-n5c6c(-c7ccccc7)cc(-c7ccccc7)cc6c6cc(-c7ccccc7)cc(-c7ccccc7)c65)c(-c5ccccc5)c4)n3)cc2)cc1 ; c1ccc(-c2ccc(-c3nc(-c4cc(-c5ccccc5)cc(-c5ccccc5)c4)nc(-c4cc(-c5ccccc5)c(-n5c6ccc(-c7ccccc7)cc6c6cc(-c7ccccc7)cc(-c7ccccc7)c65)c(-c5ccccc5)c4)n3)cc2)cc1 ; CC(C)(C)c1cc(-c2ccccc2)cc2c3cc(-c4ccccc4)ccc3n(-c3c(-c4ccccc4)cc(-c4nc(-c5ccc(-c6ccccc6)cc5)nc(-c5cc(-c6ccccc6)cc(-c6ccccc6)c5)n4)cc3-c3ccccc3)c12]

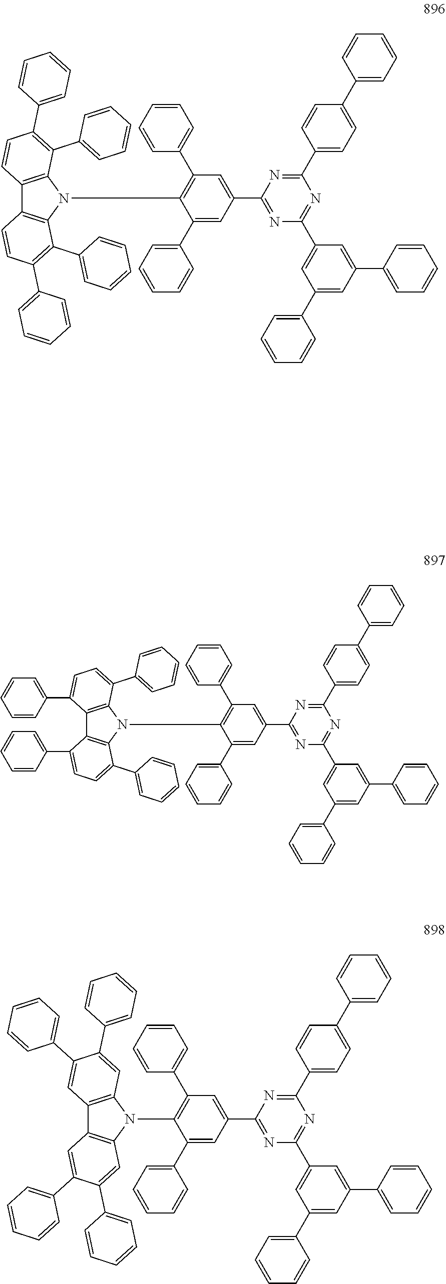 [SMILES: c1ccc(-c2ccc(-c3nc(-c4cc(-c5ccccc5)cc(-c5ccccc5)c4)nc(-c4cc(-c5ccccc5)c(-n5c6c(-c7ccccc7)c(-c7ccccc7)ccc6c6ccc(-c7ccccc7)c(-c7ccccc7)c65)c(-c5ccccc5)c4)n3)cc2)cc1 ; c1ccc(-c2ccc(-c3nc(-c4cc(-c5ccccc5)cc(-c5ccccc5)c4)nc(-c4cc(-c5ccccc5)c(-n5c6c(-c7ccccc7)ccc(-c7ccccc7)c6c6c(-c7ccccc7)ccc(-c7ccccc7)c65)c(-c5ccccc5)c4)n3)cc2)cc1 ; c1ccc(-c2ccc(-c3nc(-c4cc(-c5ccccc5)cc(-c5ccccc5)c4)nc(-c4cc(-c5ccccc5)c(-n5c6cc(-c7ccccc7)c(-c7ccccc7)cc6c6cc(-c7ccccc7)c(-c7ccccc7)cc65)c(-c5ccccc5)c4)n3)cc2)cc1]

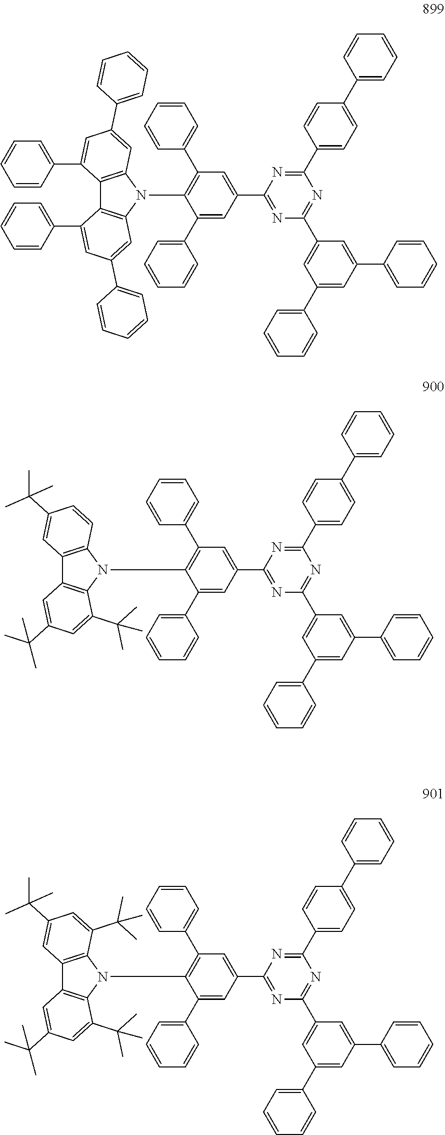 [SMILES: c1ccc(-c2ccc(-c3nc(-c4cc(-c5ccccc5)cc(-c5ccccc5)c4)nc(-c4cc(-c5ccccc5)c(-n5c6cc(-c7ccccc7)cc(-c7ccccc7)c6c6c(-c7ccccc7)cc(-c7ccccc7)cc65)c(-c5ccccc5)c4)n3)cc2)cc1 ; CC(C)(C)c1ccc2c(c1)c1cc(C(C)(C)C)cc(C(C)(C)C)c1n2-c1c(-c2ccccc2)cc(-c2nc(-c3ccc(-c4ccccc4)cc3)nc(-c3cc(-c4ccccc4)cc(-c4ccccc4)c3)n2)cc1-c1ccccc1 ; CC(C)(C)c1cc(C(C)(C)C)c2c(c1)c1cc(C(C)(C)C)cc(C(C)(C)C)c1n2-c1c(-c2ccccc2)cc(-c2nc(-c3ccc(-c4ccccc4)cc3)nc(-c3cc(-c4ccccc4)cc(-c4ccccc4)c3)n2)cc1-c1ccccc1]

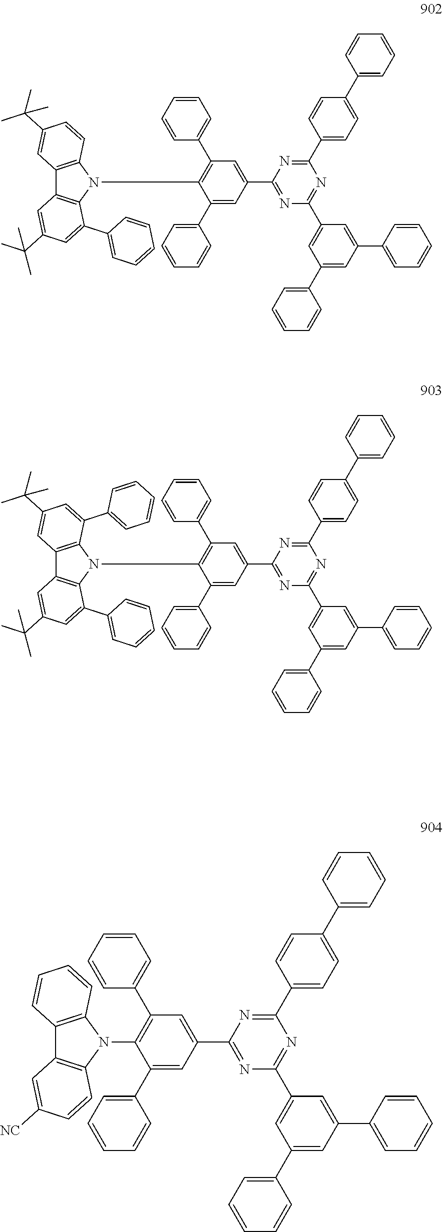 [SMILES: CC(C)(C)c1ccc2c(c1)c1cc(C(C)(C)C)cc(-c3ccccc3)c1n2-c1c(-c2ccccc2)cc(-c2nc(-c3ccc(-c4ccccc4)cc3)nc(-c3cc(-c4ccccc4)cc(-c4ccccc4)c3)n2)cc1-c1ccccc1 ; CC(C)(C)c1cc(-c2ccccc2)c2c(c1)c1cc(C(C)(C)C)cc(-c3ccccc3)c1n2-c1c(-c2ccccc2)cc(-c2nc(-c3ccc(-c4ccccc4)cc3)nc(-c3cc(-c4ccccc4)cc(-c4ccccc4)c3)n2)cc1-c1ccccc1 ; N#Cc1ccc2c(c1)c1ccccc1n2-c1c(-c2ccccc2)cc(-c2nc(-c3ccc(-c4ccccc4)cc3)nc(-c3cc(-c4ccccc4)cc(-c4ccccc4)c3)n2)cc1-c1ccccc1]

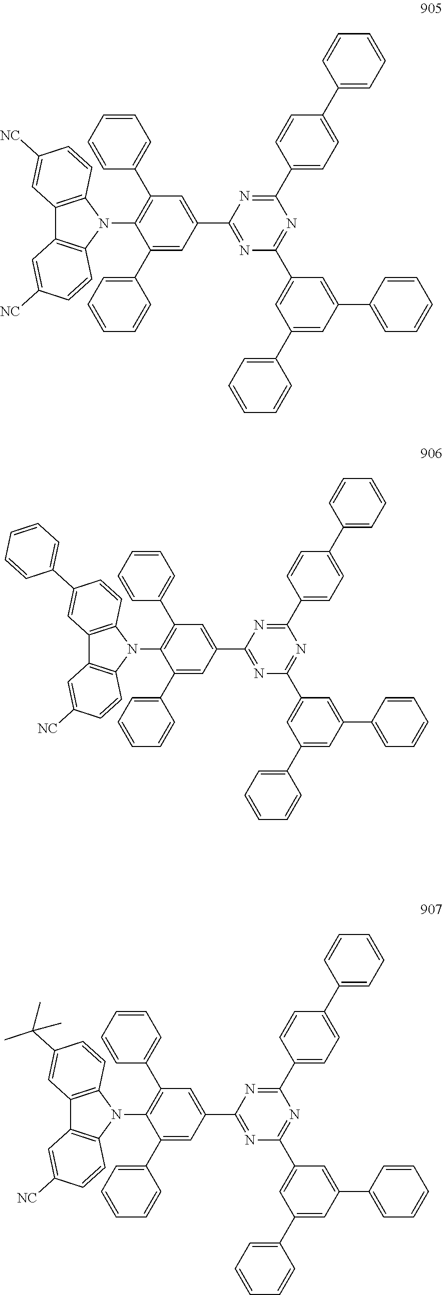 [SMILES: N#Cc1ccc2c(c1)c1cc(C#N)ccc1n2-c1c(-c2ccccc2)cc(-c2nc(-c3ccc(-c4ccccc4)cc3)nc(-c3cc(-c4ccccc4)cc(-c4ccccc4)c3)n2)cc1-c1ccccc1 ; N#Cc1ccc2c(c1)c1cc(-c3ccccc3)ccc1n2-c1c(-c2ccccc2)cc(-c2nc(-c3ccc(-c4ccccc4)cc3)nc(-c3cc(-c4ccccc4)cc(-c4ccccc4)c3)n2)cc1-c1ccccc1 ; CC(C)(C)c1ccc2c(c1)c1cc(C#N)ccc1n2-c1c(-c2ccccc2)cc(-c2nc(-c3ccc(-c4ccccc4)cc3)nc(-c3cc(-c4ccccc4)cc(-c4ccccc4)c3)n2)cc1-c1ccccc1]

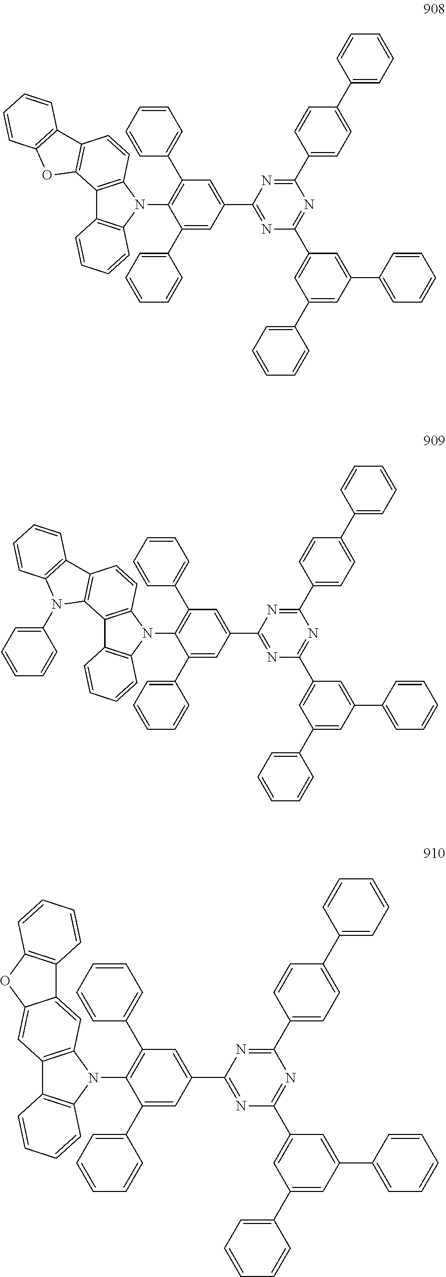 [SMILES: c1ccc(-c2ccc(-c3nc(-c4cc(-c5ccccc5)cc(-c5ccccc5)c4)nc(-c4cc(-c5ccccc5)c(-n5c6ccccc6c6c7oc8ccccc8c7ccc65)c(-c5ccccc5)c4)n3)cc2)cc1 ; c1ccc(-c2ccc(-c3nc(-c4cc(-c5ccccc5)cc(-c5ccccc5)c4)nc(-c4cc(-c5ccccc5)c(-n5c6ccccc6c6c5ccc5c7ccccc7n(-c7ccccc7)c56)c(-c5ccccc5)c4)n3)cc2)cc1 ; c1ccc(-c2ccc(-c3nc(-c4cc(-c5ccccc5)cc(-c5ccccc5)c4)nc(-c4cc(-c5ccccc5)c(-n5c6ccccc6c6cc7oc8ccccc8c7cc65)c(-c5ccccc5)c4)n3)cc2)cc1]

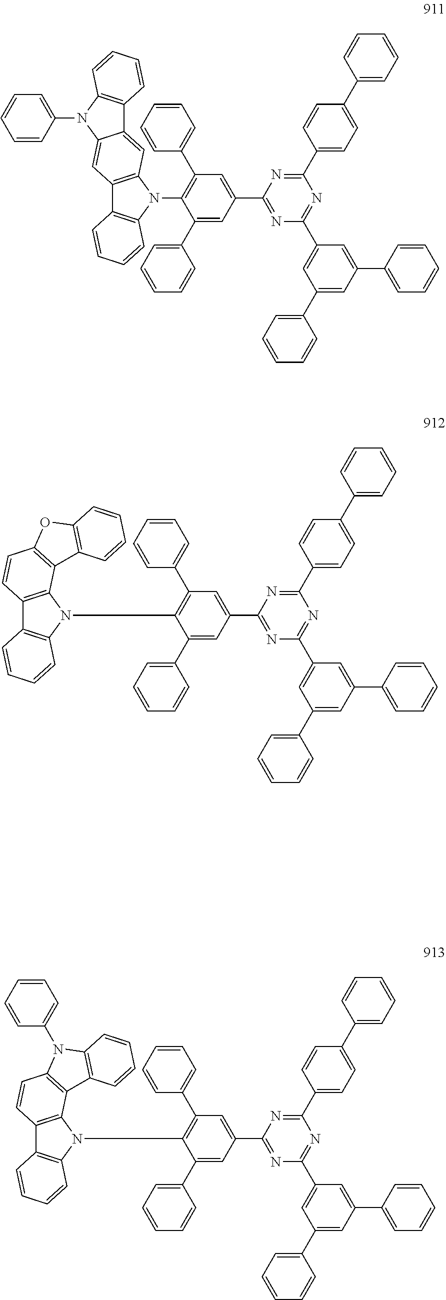 [SMILES: c1ccc(-c2ccc(-c3nc(-c4cc(-c5ccccc5)cc(-c5ccccc5)c4)nc(-c4cc(-c5ccccc5)c(-n5c6ccccc6c6cc7c(cc65)c5ccccc5n7-c5ccccc5)c(-c5ccccc5)c4)n3)cc2)cc1 ; c1ccc(-c2ccc(-c3nc(-c4cc(-c5ccccc5)cc(-c5ccccc5)c4)nc(-c4cc(-c5ccccc5)c(-n5c6ccccc6c6ccc7oc8ccccc8c7c65)c(-c5ccccc5)c4)n3)cc2)cc1 ; c1ccc(-c2ccc(-c3nc(-c4cc(-c5ccccc5)cc(-c5ccccc5)c4)nc(-c4cc(-c5ccccc5)c(-n5c6ccccc6c6ccc7c(c8ccccc8n7-c7ccccc7)c65)c(-c5ccccc5)c4)n3)cc2)cc1]

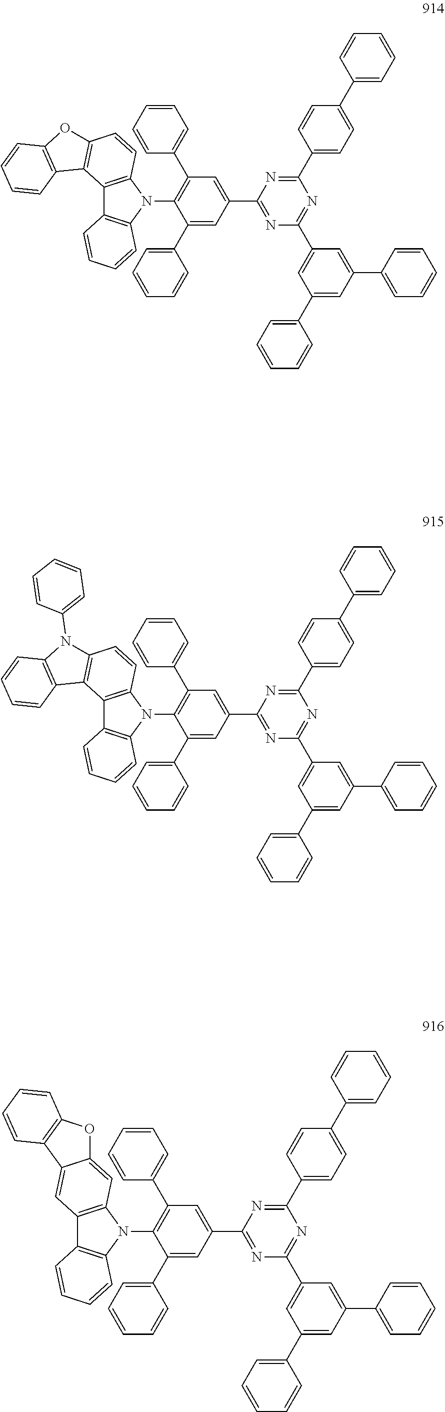 [SMILES: c1ccc(-c2ccc(-c3nc(-c4cc(-c5ccccc5)cc(-c5ccccc5)c4)nc(-c4cc(-c5ccccc5)c(-n5c6ccccc6c6c7c(ccc65)oc5ccccc57)c(-c5ccccc5)c4)n3)cc2)cc1 ; c1ccc(-c2ccc(-c3nc(-c4cc(-c5ccccc5)cc(-c5ccccc5)c4)nc(-c4cc(-c5ccccc5)c(-n5c6ccccc6c6c7c8ccccc8n(-c8ccccc8)c7ccc65)c(-c5ccccc5)c4)n3)cc2)cc1 ; c1ccc(-c2ccc(-c3nc(-c4cc(-c5ccccc5)cc(-c5ccccc5)c4)nc(-c4cc(-c5ccccc5)c(-n5c6ccccc6c6cc7c(cc65)oc5ccccc57)c(-c5ccccc5)c4)n3)cc2)cc1]

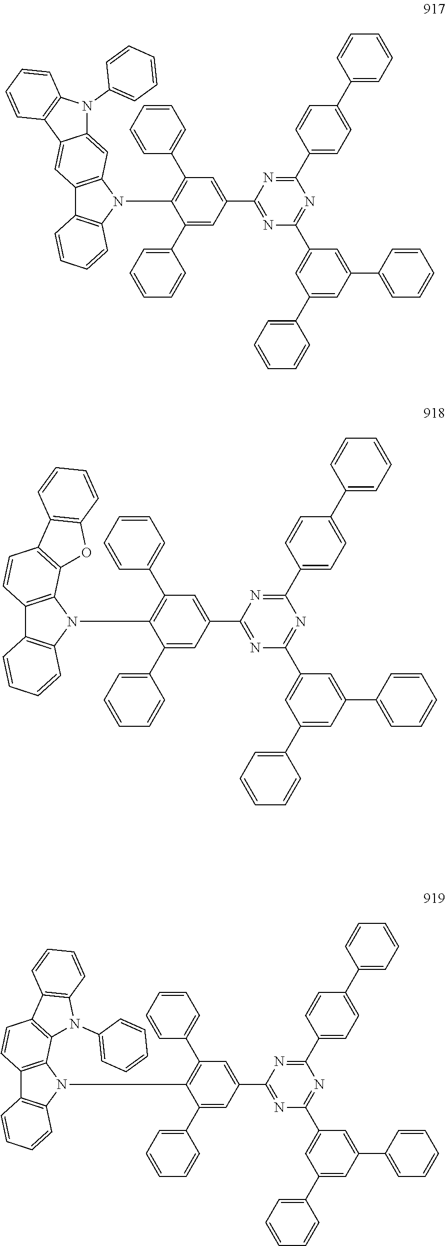 [SMILES: c1ccc(-c2ccc(-c3nc(-c4cc(-c5ccccc5)cc(-c5ccccc5)c4)nc(-c4cc(-c5ccccc5)c(-n5c6ccccc6c6cc7c8ccccc8n(-c8ccccc8)c7cc65)c(-c5ccccc5)c4)n3)cc2)cc1 ; c1ccc(-c2ccc(-c3nc(-c4cc(-c5ccccc5)cc(-c5ccccc5)c4)nc(-c4cc(-c5ccccc5)c(-n5c6ccccc6c6ccc7c8ccccc8oc7c65)c(-c5ccccc5)c4)n3)cc2)cc1 ; c1ccc(-c2ccc(-c3nc(-c4cc(-c5ccccc5)cc(-c5ccccc5)c4)nc(-c4cc(-c5ccccc5)c(-n5c6ccccc6c6ccc7c8ccccc8n(-c8ccccc8)c7c65)c(-c5ccccc5)c4)n3)cc2)cc1]

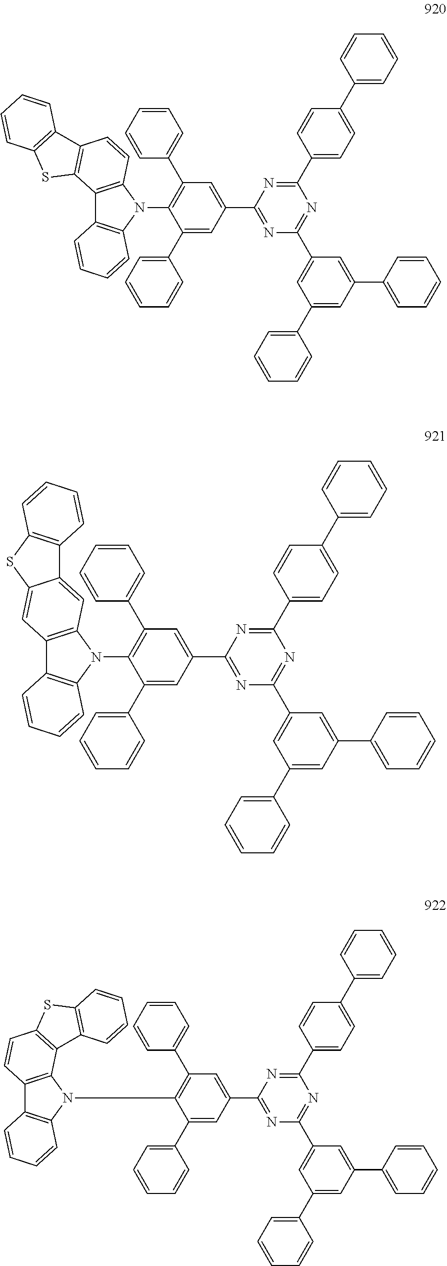 [SMILES: c1ccc(-c2ccc(-c3nc(-c4cc(-c5ccccc5)cc(-c5ccccc5)c4)nc(-c4cc(-c5ccccc5)c(-n5c6ccccc6c6c7sc8ccccc8c7ccc65)c(-c5ccccc5)c4)n3)cc2)cc1 ; c1ccc(-c2ccc(-c3nc(-c4cc(-c5ccccc5)cc(-c5ccccc5)c4)nc(-c4cc(-c5ccccc5)c(-n5c6ccccc6c6cc7sc8ccccc8c7cc65)c(-c5ccccc5)c4)n3)cc2)cc1 ; c1ccc(-c2ccc(-c3nc(-c4cc(-c5ccccc5)cc(-c5ccccc5)c4)nc(-c4cc(-c5ccccc5)c(-n5c6ccccc6c6ccc7sc8ccccc8c7c65)c(-c5ccccc5)c4)n3)cc2)cc1]

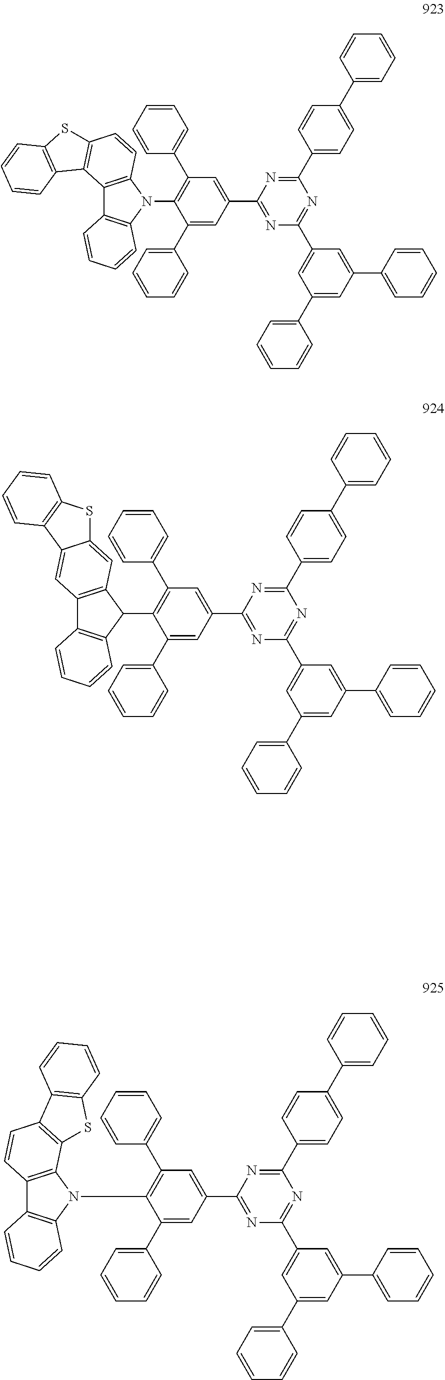 [SMILES: c1ccc(-c2ccc(-c3nc(-c4cc(-c5ccccc5)cc(-c5ccccc5)c4)nc(-c4cc(-c5ccccc5)c(-n5c6ccccc6c6c7c(ccc65)sc5ccccc57)c(-c5ccccc5)c4)n3)cc2)cc1 ; c1ccc(-c2ccc(-c3nc(-c4cc(-c5ccccc5)cc(-c5ccccc5)c4)nc(-c4cc(-c5ccccc5)c(C5c6ccccc6-c6cc7c(cc65)sc5ccccc57)c(-c5ccccc5)c4)n3)cc2)cc1 ; c1ccc(-c2ccc(-c3nc(-c4cc(-c5ccccc5)cc(-c5ccccc5)c4)nc(-c4cc(-c5ccccc5)c(-n5c6ccccc6c6ccc7c8ccccc8sc7c65)c(-c5ccccc5)c4)n3)cc2)cc1]

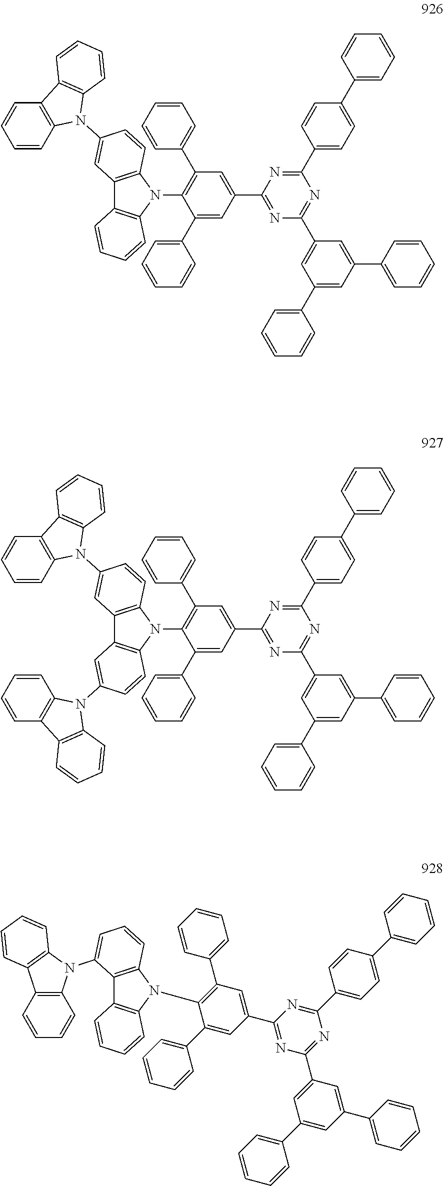 [SMILES: c1ccc(-c2ccc(-c3nc(-c4cc(-c5ccccc5)cc(-c5ccccc5)c4)nc(-c4cc(-c5ccccc5)c(-n5c6ccccc6c6cc(-n7c8ccccc8c8ccccc87)ccc65)c(-c5ccccc5)c4)n3)cc2)cc1 ; c1ccc(-c2ccc(-c3nc(-c4cc(-c5ccccc5)cc(-c5ccccc5)c4)nc(-c4cc(-c5ccccc5)c(-n5c6ccc(-n7c8ccccc8c8ccccc87)cc6c6cc(-n7c8ccccc8c8ccccc87)ccc65)c(-c5ccccc5)c4)n3)cc2)cc1 ; c1ccc(-c2ccc(-c3nc(-c4cc(-c5ccccc5)cc(-c5ccccc5)c4)nc(-c4cc(-c5ccccc5)c(-n5c6ccccc6c6c(-n7c8ccccc8c8ccccc87)cccc65)c(-c5ccccc5)c4)n3)cc2)cc1]

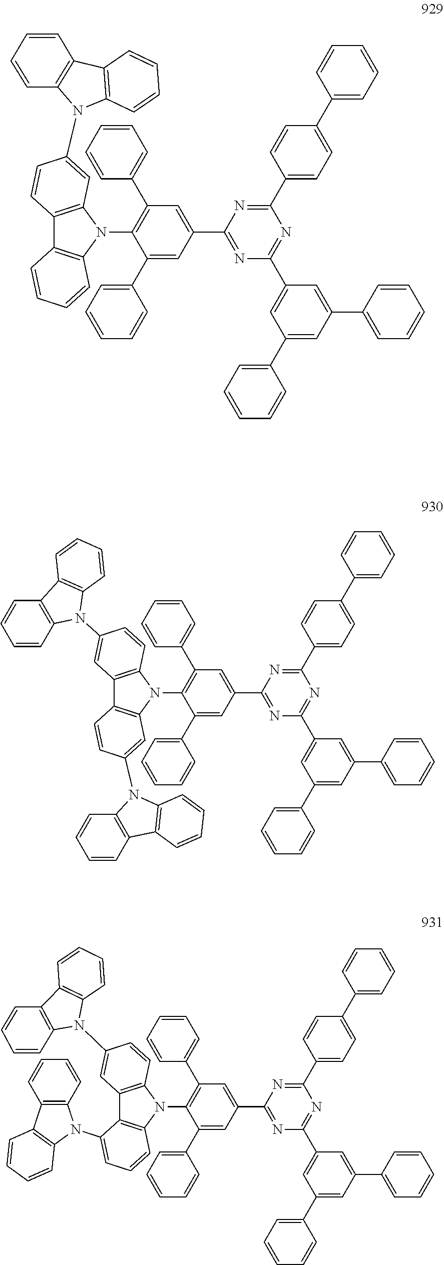 [SMILES: c1ccc(-c2ccc(-c3nc(-c4cc(-c5ccccc5)cc(-c5ccccc5)c4)nc(-c4cc(-c5ccccc5)c(-n5c6ccccc6c6ccc(-n7c8ccccc8c8ccccc87)cc65)c(-c5ccccc5)c4)n3)cc2)cc1 ; c1ccc(-c2ccc(-c3nc(-c4cc(-c5ccccc5)cc(-c5ccccc5)c4)nc(-c4cc(-c5ccccc5)c(-n5c6ccc(-n7c8ccccc8c8ccccc87)cc6c6ccc(-n7c8ccccc8c8ccccc87)cc65)c(-c5ccccc5)c4)n3)cc2)cc1 ; c1ccc(-c2ccc(-c3nc(-c4cc(-c5ccccc5)cc(-c5ccccc5)c4)nc(-c4cc(-c5ccccc5)c(-n5c6ccc(-n7c8ccccc8c8ccccc87)cc6c6c(-n7c8ccccc8c8ccccc87)cccc65)c(-c5ccccc5)c4)n3)cc2)cc1]

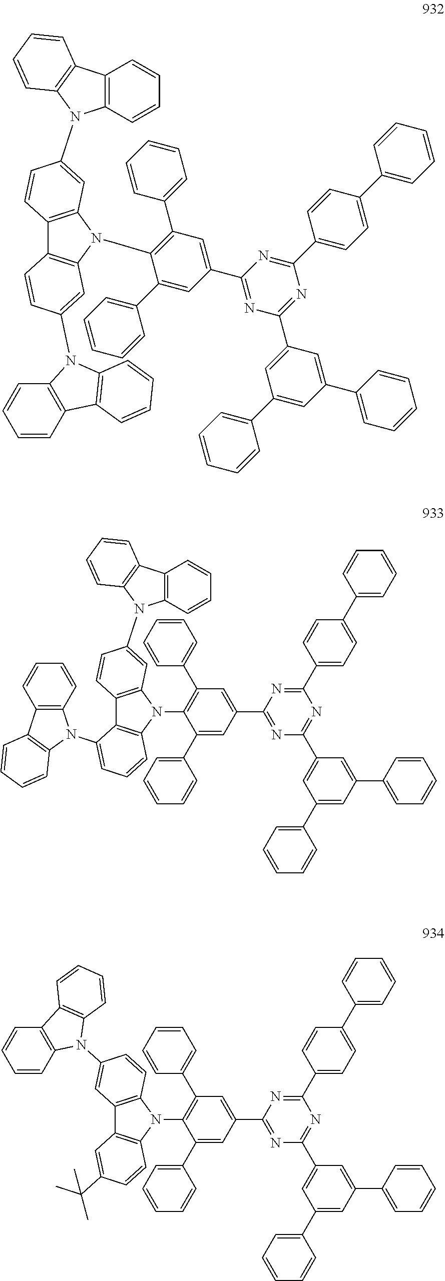 [SMILES: c1ccc(-c2ccc(-c3nc(-c4cc(-c5ccccc5)cc(-c5ccccc5)c4)nc(-c4cc(-c5ccccc5)c(Cn5c6cc(-n7c8ccccc8c8ccccc87)ccc6c6ccc(-n7c8ccccc8c8ccccc87)cc65)c(-c5ccccc5)c4)n3)cc2)cc1 ; c1ccc(-c2ccc(-c3nc(-c4cc(-c5ccccc5)cc(-c5ccccc5)c4)nc(-c4cc(-c5ccccc5)c(-n5c6cc(-n7c8ccccc8c8ccccc87)ccc6c6c(-n7c8ccccc8c8ccccc87)cccc65)c(-c5ccccc5)c4)n3)cc2)cc1 ; CC(C)(C)c1ccc2c(c1)c1cc(-n3c4ccccc4c4ccccc43)ccc1n2-c1c(-c2ccccc2)cc(-c2nc(-c3ccc(-c4ccccc4)cc3)nc(-c3cc(-c4ccccc4)cc(-c4ccccc4)c3)n2)cc1-c1ccccc1]

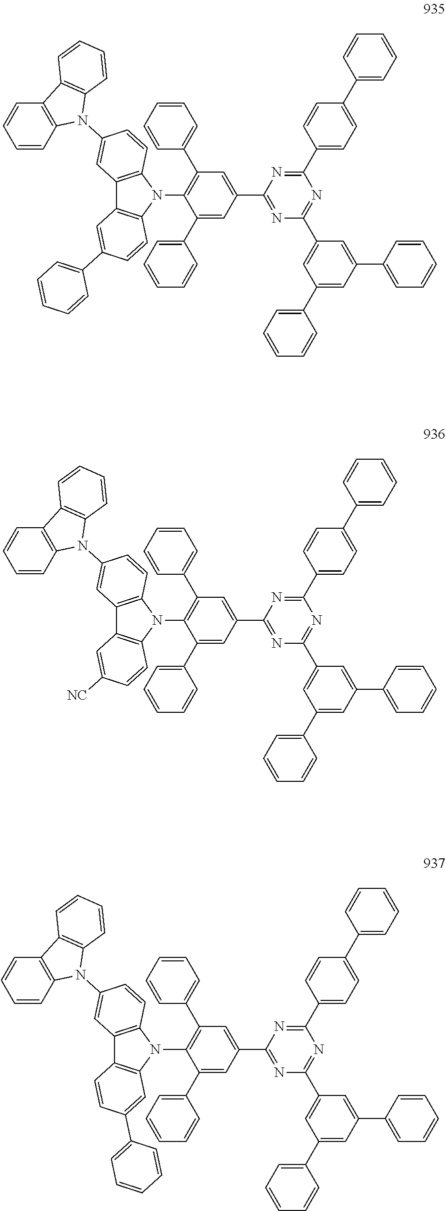 [SMILES: c1ccc(-c2ccc(-c3nc(-c4cc(-c5ccccc5)cc(-c5ccccc5)c4)nc(-c4cc(-c5ccccc5)c(-n5c6ccc(-c7ccccc7)cc6c6cc(-n7c8ccccc8c8ccccc87)ccc65)c(-c5ccccc5)c4)n3)cc2)cc1 ; N#Cc1ccc2c(c1)c1cc(-n3c4ccccc4c4ccccc43)ccc1n2-c1c(-c2ccccc2)cc(-c2nc(-c3ccc(-c4ccccc4)cc3)nc(-c3cc(-c4ccccc4)cc(-c4ccccc4)c3)n2)cc1-c1ccccc1 ; c1ccc(-c2ccc(-c3nc(-c4cc(-c5ccccc5)cc(-c5ccccc5)c4)nc(-c4cc(-c5ccccc5)c(-n5c6ccc(-n7c8ccccc8c8ccccc87)cc6c6ccc(-c7ccccc7)cc65)c(-c5ccccc5)c4)n3)cc2)cc1]

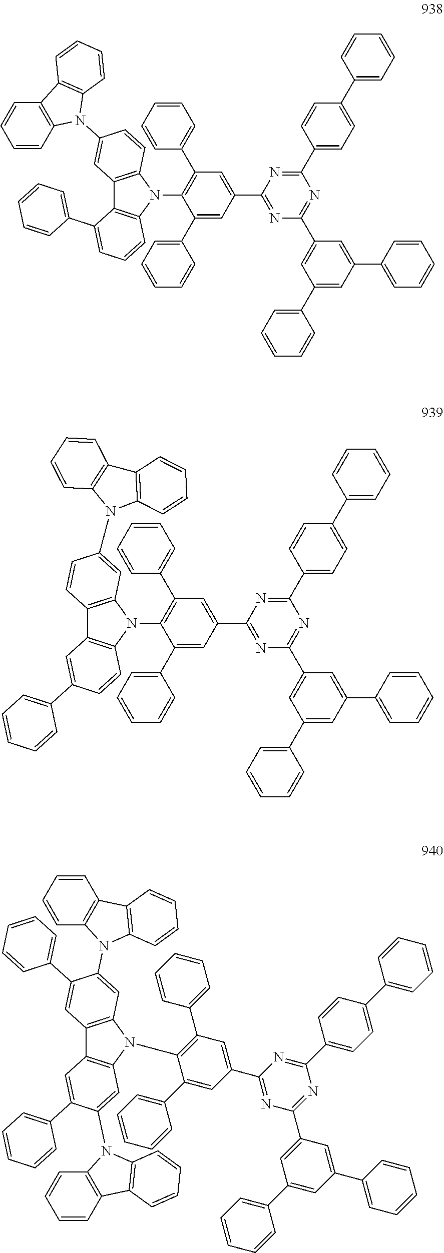 [SMILES: c1ccc(-c2ccc(-c3nc(-c4cc(-c5ccccc5)cc(-c5ccccc5)c4)nc(-c4cc(-c5ccccc5)c(-n5c6ccc(-n7c8ccccc8c8ccccc87)cc6c6c(-c7ccccc7)cccc65)c(-c5ccccc5)c4)n3)cc2)cc1 ; c1ccc(-c2ccc(-c3nc(-c4cc(-c5ccccc5)cc(-c5ccccc5)c4)nc(-c4cc(-c5ccccc5)c(-n5c6ccc(-c7ccccc7)cc6c6ccc(-n7c8ccccc8c8ccccc87)cc65)c(-c5ccccc5)c4)n3)cc2)cc1 ; c1ccc(-c2ccc(-c3nc(-c4cc(-c5ccccc5)cc(-c5ccccc5)c4)nc(-c4cc(-c5ccccc5)c(-n5c6cc(-n7c8ccccc8c8ccccc87)c(-c7ccccc7)cc6c6cc(-c7ccccc7)c(-n7c8ccccc8c8ccccc87)cc65)c(-c5ccccc5)c4)n3)cc2)cc1]

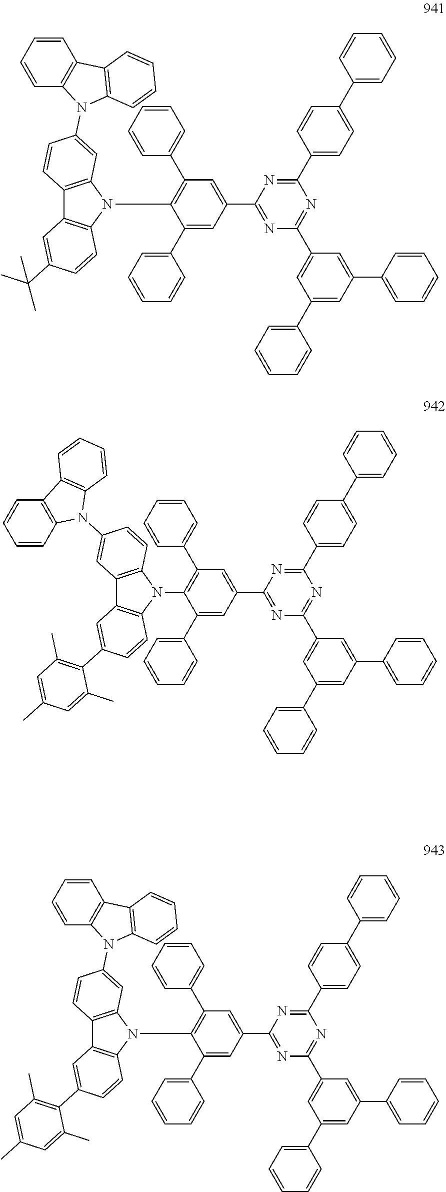 [SMILES: CC(C)(C)c1ccc2c(c1)c1ccc(-n3c4ccccc4c4ccccc43)cc1n2-c1c(-c2ccccc2)cc(-c2nc(-c3ccc(-c4ccccc4)cc3)nc(-c3cc(-c4ccccc4)cc(-c4ccccc4)c3)n2)cc1-c1ccccc1 ; Cc1cc(C)c(-c2ccc3c(c2)c2cc(-n4c5ccccc5c5ccccc54)ccc2n3-c2c(-c3ccccc3)cc(-c3nc(-c4ccc(-c5ccccc5)cc4)nc(-c4cc(-c5ccccc5)cc(-c5ccccc5)c4)n3)cc2-c2ccccc2)c(C)c1 ; Cc1cc(C)c(-c2ccc3c(c2)c2ccc(-n4c5ccccc5c5ccccc54)cc2n3-c2c(-c3ccccc3)cc(-c3nc(-c4ccc(-c5ccccc5)cc4)nc(-c4cc(-c5ccccc5)cc(-c5ccccc5)c4)n3)cc2-c2ccccc2)c(C)c1]

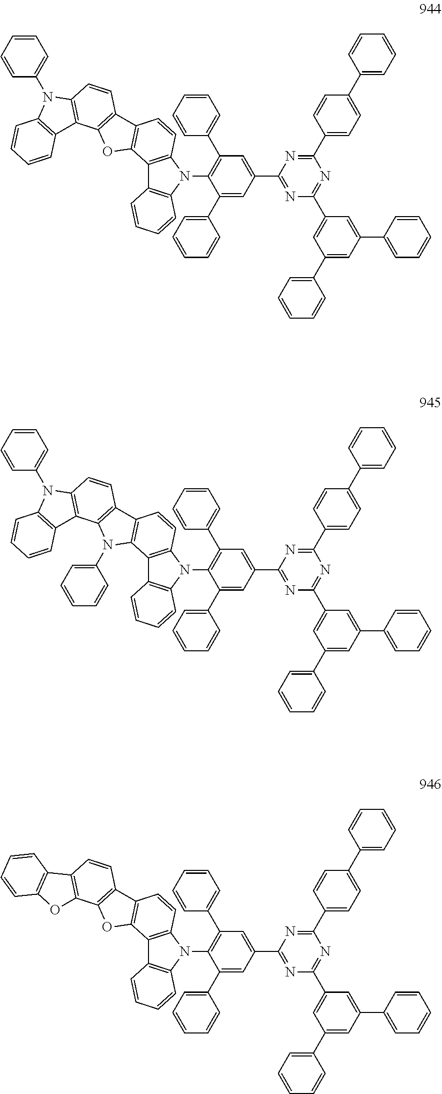 [SMILES: c1ccc(-c2ccc(-c3nc(-c4cc(-c5ccccc5)cc(-c5ccccc5)c4)nc(-c4cc(-c5ccccc5)c(-n5c6ccccc6c6c7oc8c(ccc9c8c8ccccc8n9-c8ccccc8)c7ccc65)c(-c5ccccc5)c4)n3)cc2)cc1 ; c1ccc(-c2ccc(-c3nc(-c4cc(-c5ccccc5)cc(-c5ccccc5)c4)nc(-c4cc(-c5ccccc5)c(-n5c6ccccc6c6c5ccc5c7ccc8c(c9ccccc9n8-c8ccccc8)c7n(-c7ccccc7)c56)c(-c5ccccc5)c4)n3)cc2)cc1 ; c1ccc(-c2ccc(-c3nc(-c4cc(-c5ccccc5)cc(-c5ccccc5)c4)nc(-c4cc(-c5ccccc5)c(-n5c6ccccc6c6c7oc8c(ccc9c%10ccccc%10oc98)c7ccc65)c(-c5ccccc5)c4)n3)cc2)cc1]

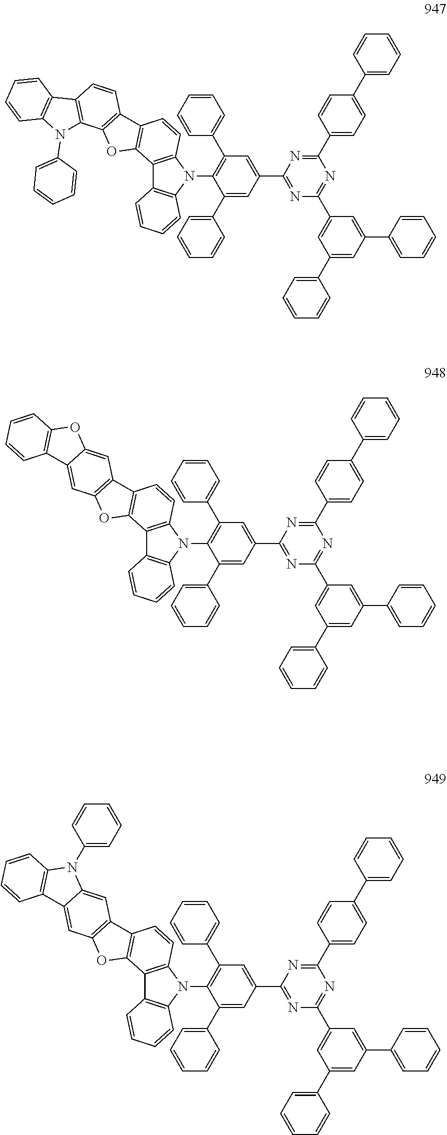 [SMILES: c1ccc(-c2ccc(-c3nc(-c4cc(-c5ccccc5)cc(-c5ccccc5)c4)nc(-c4cc(-c5ccccc5)c(-n5c6ccccc6c6c7oc8c(ccc9c%10ccccc%10n(-c%10ccccc%10)c98)c7ccc65)c(-c5ccccc5)c4)n3)cc2)cc1 ; c1ccc(-c2ccc(-c3nc(-c4cc(-c5ccccc5)cc(-c5ccccc5)c4)nc(-c4cc(-c5ccccc5)c(-n5c6ccccc6c6c7oc8cc9c(cc8c7ccc65)oc5ccccc59)c(-c5ccccc5)c4)n3)cc2)cc1 ; c1ccc(-c2ccc(-c3nc(-c4cc(-c5ccccc5)cc(-c5ccccc5)c4)nc(-c4cc(-c5ccccc5)c(-n5c6ccccc6c6c7oc8cc9c%10ccccc%10n(-c%10ccccc%10)c9cc8c7ccc65)c(-c5ccccc5)c4)n3)cc2)cc1]

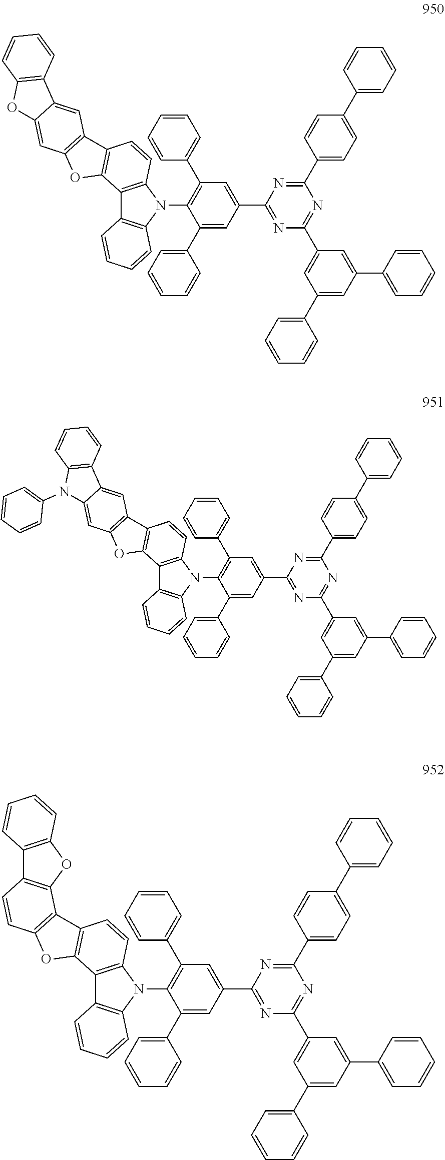 [SMILES: c1ccc(-c2ccc(-c3nc(-c4cc(-c5ccccc5)cc(-c5ccccc5)c4)nc(-c4cc(-c5ccccc5)c(-n5c6ccccc6c6c7oc8cc9oc%10ccccc%10c9cc8c7ccc65)c(-c5ccccc5)c4)n3)cc2)cc1 ; c1ccc(-c2ccc(-c3nc(-c4cc(-c5ccccc5)cc(-c5ccccc5)c4)nc(-c4cc(-c5ccccc5)c(-n5c6ccccc6c6c7oc8cc9c(cc8c7ccc65)c5ccccc5n9-c5ccccc5)c(-c5ccccc5)c4)n3)cc2)cc1 ; c1ccc(-c2ccc(-c3nc(-c4cc(-c5ccccc5)cc(-c5ccccc5)c4)nc(-c4cc(-c5ccccc5)c(-n5c6ccccc6c6c7oc8ccc9c%10ccccc%10oc9c8c7ccc65)c(-c5ccccc5)c4)n3)cc2)cc1]

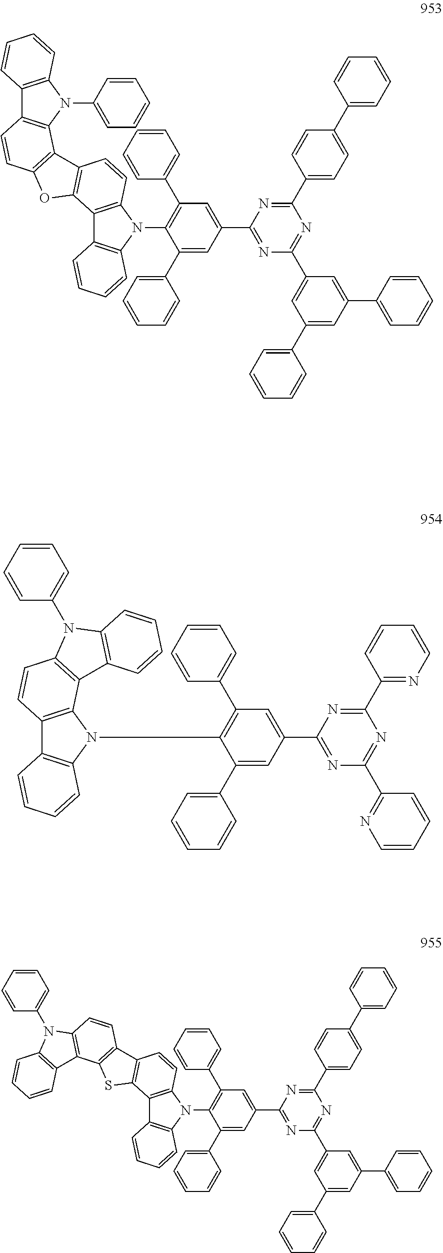 [SMILES: c1ccc(-c2ccc(-c3nc(-c4cc(-c5ccccc5)cc(-c5ccccc5)c4)nc(-c4cc(-c5ccccc5)c(-n5c6ccccc6c6c7oc8ccc9c%10ccccc%10n(-c%10ccccc%10)c9c8c7ccc65)c(-c5ccccc5)c4)n3)cc2)cc1 ; c1ccc(-c2cc(-c3nc(-c4ccccn4)nc(-c4ccccn4)n3)cc(-c3ccccc3)c2-n2c3ccccc3c3ccc4c(c5ccccc5n4-c4ccccc4)c32)cc1 ; c1ccc(-c2ccc(-c3nc(-c4cc(-c5ccccc5)cc(-c5ccccc5)c4)nc(-c4cc(-c5ccccc5)c(-n5c6ccccc6c6c7sc8c(ccc9c8c8ccccc8n9-c8ccccc8)c7ccc65)c(-c5ccccc5)c4)n3)cc2)cc1]

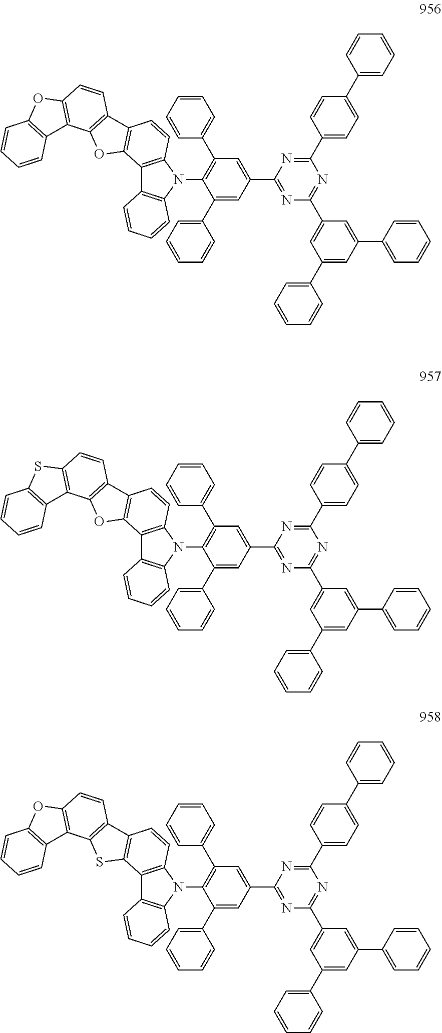 [SMILES: c1ccc(-c2ccc(-c3nc(-c4cc(-c5ccccc5)cc(-c5ccccc5)c4)nc(-c4cc(-c5ccccc5)c(-n5c6ccccc6c6c7oc8c(ccc9oc%10ccccc%10c98)c7ccc65)c(-c5ccccc5)c4)n3)cc2)cc1 ; c1ccc(-c2ccc(-c3nc(-c4cc(-c5ccccc5)cc(-c5ccccc5)c4)nc(-c4cc(-c5ccccc5)c(-n5c6ccccc6c6c7oc8c(ccc9sc%10ccccc%10c98)c7ccc65)c(-c5ccccc5)c4)n3)cc2)cc1 ; c1ccc(-c2ccc(-c3nc(-c4cc(-c5ccccc5)cc(-c5ccccc5)c4)nc(-c4cc(-c5ccccc5)c(-n5c6ccccc6c6c7sc8c(ccc9oc%10ccccc%10c98)c7ccc65)c(-c5ccccc5)c4)n3)cc2)cc1]

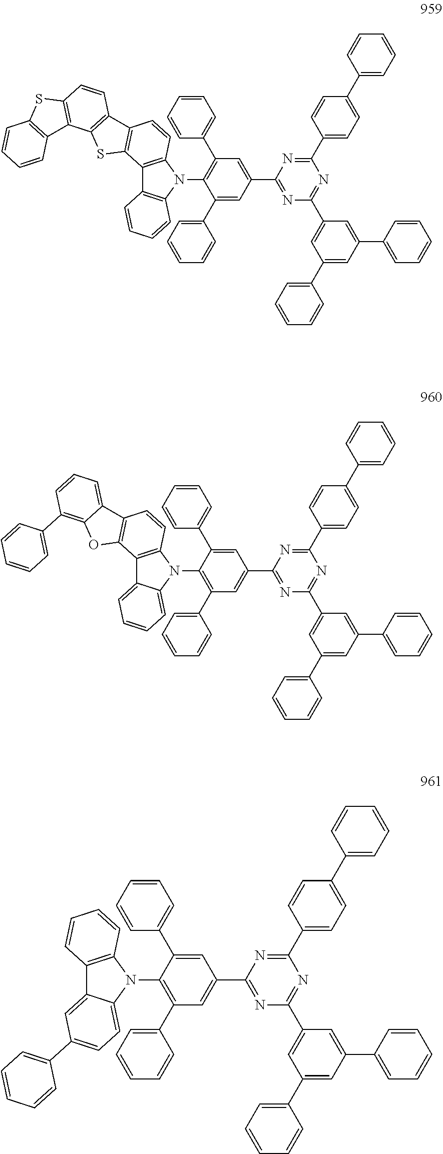 [SMILES: c1ccc(-c2ccc(-c3nc(-c4cc(-c5ccccc5)cc(-c5ccccc5)c4)nc(-c4cc(-c5ccccc5)c(-n5c6ccccc6c6c7sc8c(ccc9sc%10ccccc%10c98)c7ccc65)c(-c5ccccc5)c4)n3)cc2)cc1 ; c1ccc(-c2ccc(-c3nc(-c4cc(-c5ccccc5)cc(-c5ccccc5)c4)nc(-c4cc(-c5ccccc5)c(-n5c6ccccc6c6c7oc8c(-c9ccccc9)cccc8c7ccc65)c(-c5ccccc5)c4)n3)cc2)cc1 ; c1ccc(-c2ccc(-c3nc(-c4cc(-c5ccccc5)cc(-c5ccccc5)c4)nc(-c4cc(-c5ccccc5)c(-n5c6ccccc6c6cc(-c7ccccc7)ccc65)c(-c5ccccc5)c4)n3)cc2)cc1]

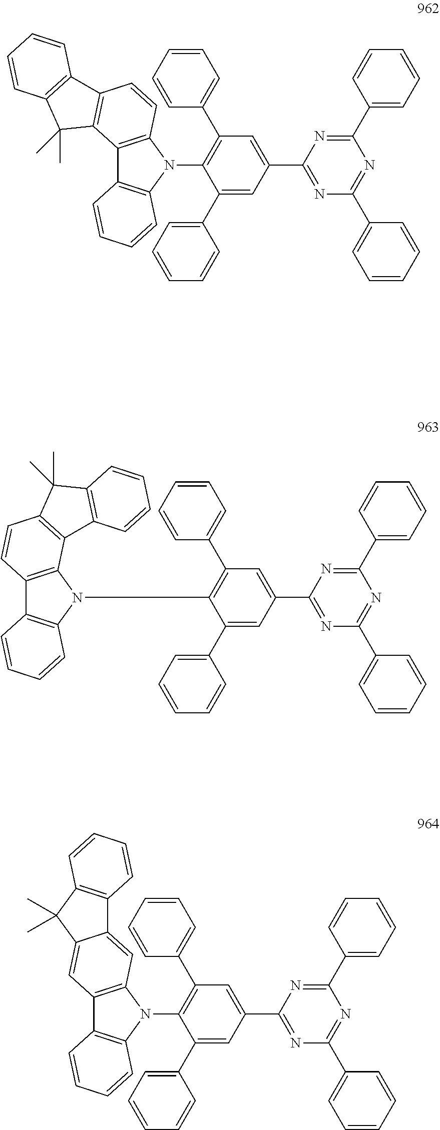 [SMILES: CC1(C)c2ccccc2-c2ccc3c(c21)c1ccccc1n3-c1c(-c2ccccc2)cc(-c2nc(-c3ccccc3)nc(-c3ccccc3)n2)cc1-c1ccccc1 ; CC1(C)c2ccccc2-c2c1ccc1c3ccccc3n(-c3c(-c4ccccc4)cc(-c4nc(-c5ccccc5)nc(-c5ccccc5)n4)cc3-c3ccccc3)c21 ; CC1(C)c2ccccc2-c2cc3c(cc21)c1ccccc1n3-c1c(-c2ccccc2)cc(-c2nc(-c3ccccc3)nc(-c3ccccc3)n2)cc1-c1ccccc1]

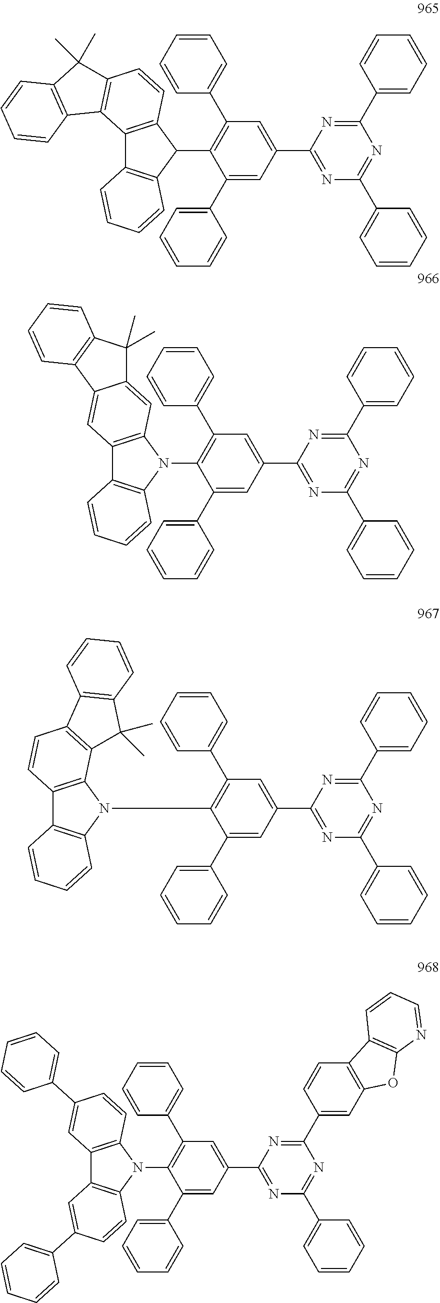 [SMILES: CC1(C)c2ccccc2-c2c1ccc1c2-c2ccccc2C1c1c(-c2ccccc2)cc(-c2nc(-c3ccccc3)nc(-c3ccccc3)n2)cc1-c1ccccc1 ; CC1(C)c2ccccc2-c2cc3c4ccccc4n(-c4c(-c5ccccc5)cc(-c5nc(-c6ccccc6)nc(-c6ccccc6)n5)cc4-c4ccccc4)c3cc21 ; CC1(C)c2ccccc2-c2ccc3c4ccccc4n(-c4c(-c5ccccc5)cc(-c5nc(-c6ccccc6)nc(-c6ccccc6)n5)cc4-c4ccccc4)c3c21 ; c1ccc(-c2ccc3c(c2)c2cc(-c4ccccc4)ccc2n3-c2c(-c3ccccc3)cc(-c3nc(-c4ccccc4)nc(-c4ccc5c(c4)oc4ncccc45)n3)cc2-c2ccccc2)cc1]

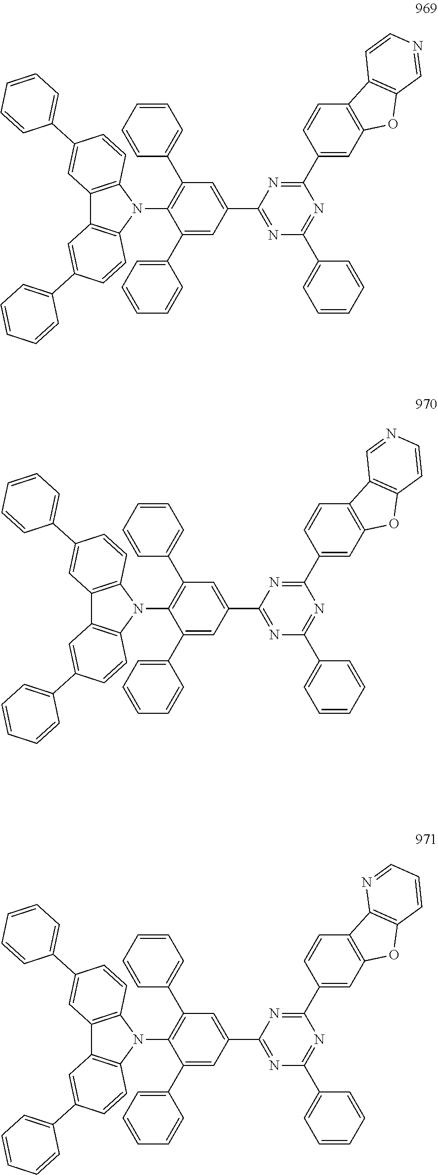 [SMILES: c1ccc(-c2ccc3c(c2)c2cc(-c4ccccc4)ccc2n3-c2c(-c3ccccc3)cc(-c3nc(-c4ccccc4)nc(-c4ccc5c(c4)oc4cnccc45)n3)cc2-c2ccccc2)cc1 ; c1ccc(-c2ccc3c(c2)c2cc(-c4ccccc4)ccc2n3-c2c(-c3ccccc3)cc(-c3nc(-c4ccccc4)nc(-c4ccc5c(c4)oc4ccncc45)n3)cc2-c2ccccc2)cc1 ; c1ccc(-c2ccc3c(c2)c2cc(-c4ccccc4)ccc2n3-c2c(-c3ccccc3)cc(-c3nc(-c4ccccc4)nc(-c4ccc5c(c4)oc4cccnc45)n3)cc2-c2ccccc2)cc1]

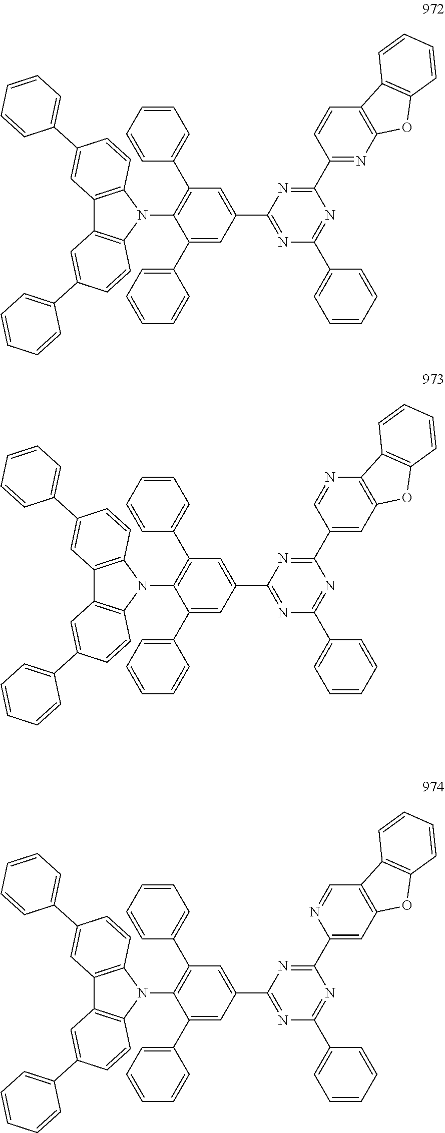 [SMILES: c1ccc(-c2ccc3c(c2)c2cc(-c4ccccc4)ccc2n3-c2c(-c3ccccc3)cc(-c3nc(-c4ccccc4)nc(-c4ccc5c(n4)oc4ccccc45)n3)cc2-c2ccccc2)cc1 ; c1ccc(-c2ccc3c(c2)c2cc(-c4ccccc4)ccc2n3-c2c(-c3ccccc3)cc(-c3nc(-c4ccccc4)nc(-c4cnc5c(c4)oc4ccccc45)n3)cc2-c2ccccc2)cc1 ; c1ccc(-c2ccc3c(c2)c2cc(-c4ccccc4)ccc2n3-c2c(-c3ccccc3)cc(-c3nc(-c4ccccc4)nc(-c4cc5oc6ccccc6c5cn4)n3)cc2-c2ccccc2)cc1]

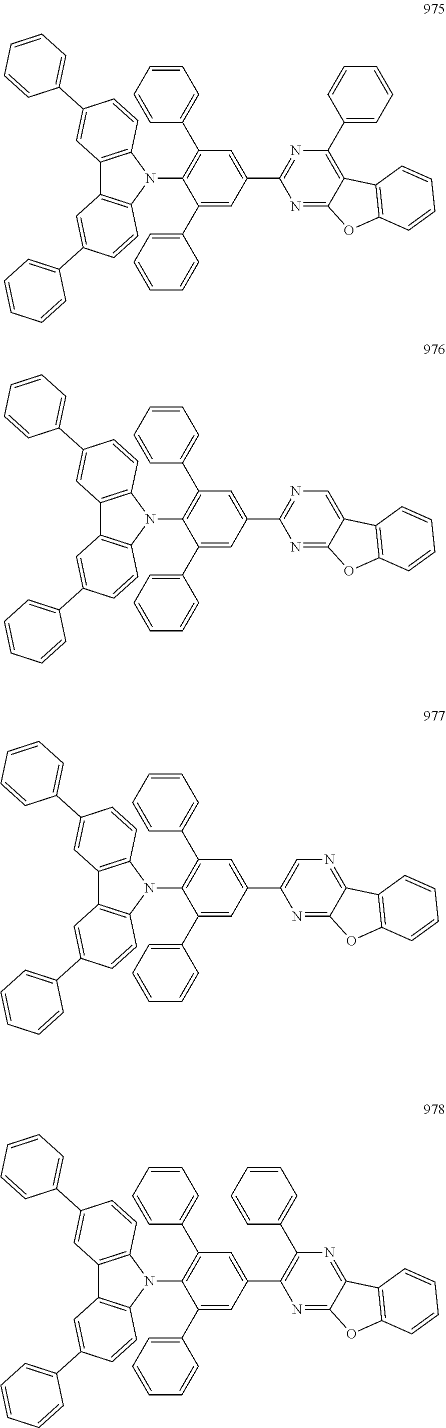 [SMILES: c1ccc(-c2ccc3c(c2)c2cc(-c4ccccc4)ccc2n3-c2c(-c3ccccc3)cc(-c3nc(-c4ccccc4)c4c(n3)oc3ccccc34)cc2-c2ccccc2)cc1 ; c1ccc(-c2ccc3c(c2)c2cc(-c4ccccc4)ccc2n3-c2c(-c3ccccc3)cc(-c3ncc4c(n3)oc3ccccc34)cc2-c2ccccc2)cc1 ; c1ccc(-c2ccc3c(c2)c2cc(-c4ccccc4)ccc2n3-c2c(-c3ccccc3)cc(-c3cnc4c(n3)oc3ccccc34)cc2-c2ccccc2)cc1 ; c1ccc(-c2ccc3c(c2)c2cc(-c4ccccc4)ccc2n3-c2c(-c3ccccc3)cc(-c3nc4oc5ccccc5c4nc3-c3ccccc3)cc2-c2ccccc2)cc1]

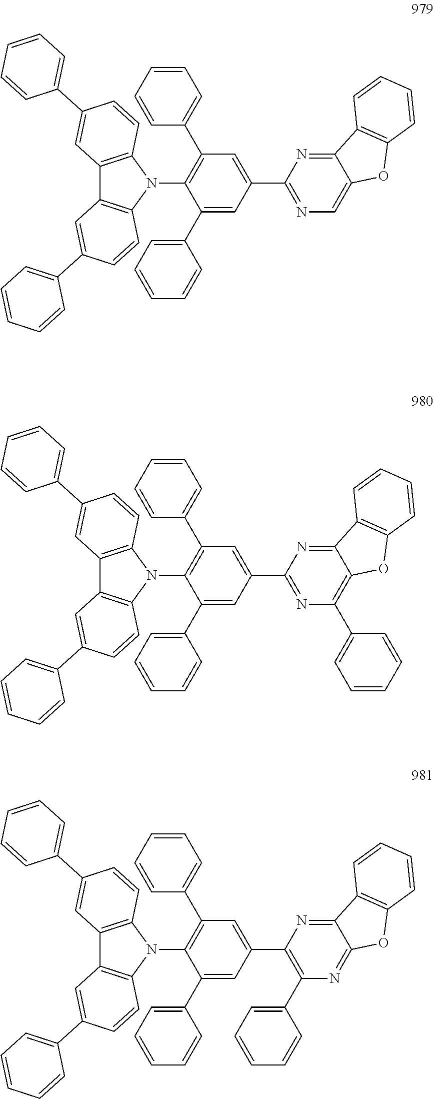 [SMILES: c1ccc(-c2ccc3c(c2)c2cc(-c4ccccc4)ccc2n3-c2c(-c3ccccc3)cc(-c3ncc4oc5ccccc5c4n3)cc2-c2ccccc2)cc1 ; c1ccc(-c2ccc3c(c2)c2cc(-c4ccccc4)ccc2n3-c2c(-c3ccccc3)cc(-c3nc(-c4ccccc4)c4oc5ccccc5c4n3)cc2-c2ccccc2)cc1 ; c1ccc(-c2ccc3c(c2)c2cc(-c4ccccc4)ccc2n3-c2c(-c3ccccc3)cc(-c3nc4c(nc3-c3ccccc3)oc3ccccc34)cc2-c2ccccc2)cc1]

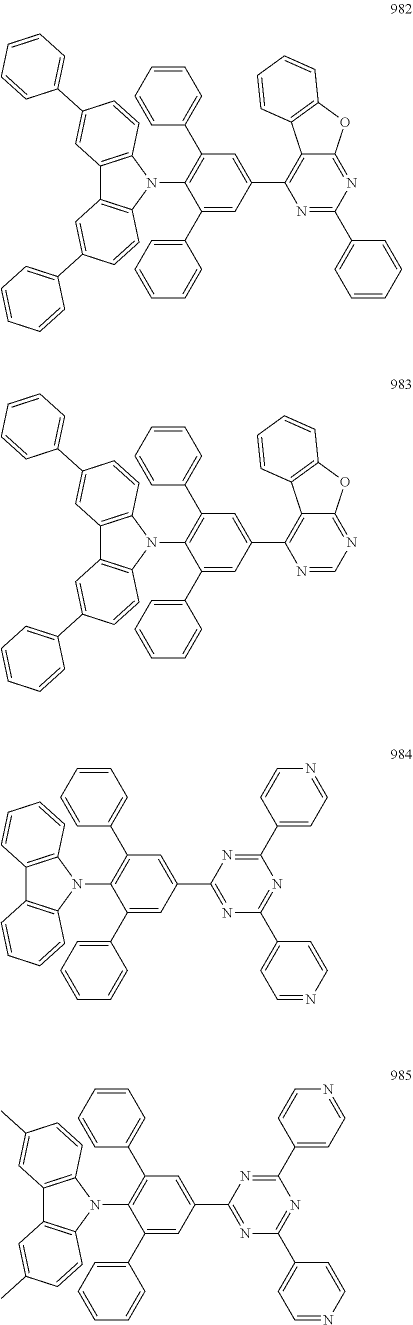 [SMILES: c1ccc(-c2ccc3c(c2)c2cc(-c4ccccc4)ccc2n3-c2c(-c3ccccc3)cc(-c3nc(-c4ccccc4)nc4oc5ccccc5c34)cc2-c2ccccc2)cc1 ; c1ccc(-c2ccc3c(c2)c2cc(-c4ccccc4)ccc2n3-c2c(-c3ccccc3)cc(-c3ncnc4oc5ccccc5c34)cc2-c2ccccc2)cc1 ; c1ccc(-c2cc(-c3nc(-c4ccncc4)nc(-c4ccncc4)n3)cc(-c3ccccc3)c2-n2c3ccccc3c3ccccc32)cc1 ; Cc1ccc2c(c1)c1cc(C)ccc1n2-c1c(-c2ccccc2)cc(-c2nc(-c3ccncc3)nc(-c3ccncc3)n2)cc1-c1ccccc1]

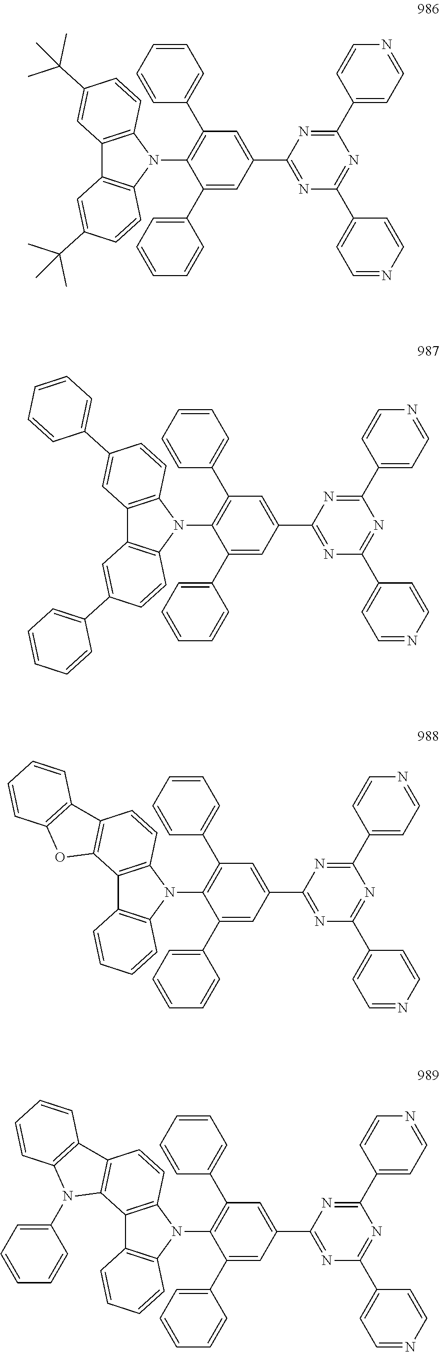 [SMILES: CC(C)(C)c1ccc2c(c1)c1cc(C(C)(C)C)ccc1n2-c1c(-c2ccccc2)cc(-c2nc(-c3ccncc3)nc(-c3ccncc3)n2)cc1-c1ccccc1 ; c1ccc(-c2ccc3c(c2)c2cc(-c4ccccc4)ccc2n3-c2c(-c3ccccc3)cc(-c3nc(-c4ccncc4)nc(-c4ccncc4)n3)cc2-c2ccccc2)cc1 ; c1ccc(-c2cc(-c3nc(-c4ccncc4)nc(-c4ccncc4)n3)cc(-c3ccccc3)c2-n2c3ccccc3c3c4oc5ccccc5c4ccc32)cc1 ; c1ccc(-c2cc(-c3nc(-c4ccncc4)nc(-c4ccncc4)n3)cc(-c3ccccc3)c2-n2c3ccccc3c3c2ccc2c4ccccc4n(-c4ccccc4)c23)cc1]

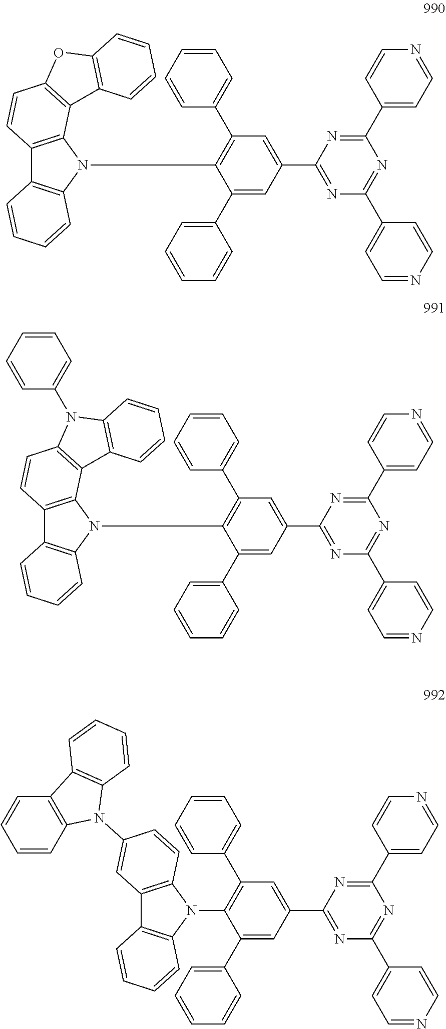 [SMILES: c1ccc(-c2cc(-c3nc(-c4ccncc4)nc(-c4ccncc4)n3)cc(-c3ccccc3)c2-n2c3ccccc3c3ccc4oc5ccccc5c4c32)cc1 ; c1ccc(-c2cc(-c3nc(-c4ccncc4)nc(-c4ccncc4)n3)cc(-c3ccccc3)c2-n2c3ccccc3c3ccc4c(c5ccccc5n4-c4ccccc4)c32)cc1 ; c1ccc(-c2cc(-c3nc(-c4ccncc4)nc(-c4ccncc4)n3)cc(-c3ccccc3)c2-n2c3ccccc3c3cc(-n4c5ccccc5c5ccccc54)ccc32)cc1]

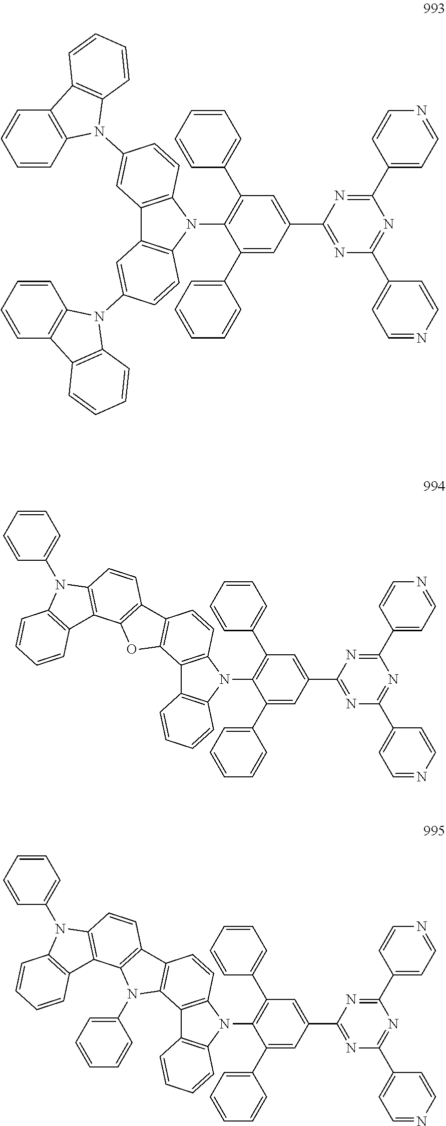 [SMILES: c1ccc(-c2cc(-c3nc(-c4ccncc4)nc(-c4ccncc4)n3)cc(-c3ccccc3)c2-n2c3ccc(-n4c5ccccc5c5ccccc54)cc3c3cc(-n4c5ccccc5c5ccccc54)ccc32)cc1 ; c1ccc(-c2cc(-c3nc(-c4ccncc4)nc(-c4ccncc4)n3)cc(-c3ccccc3)c2-n2c3ccccc3c3c4oc5c(ccc6c5c5ccccc5n6-c5ccccc5)c4ccc32)cc1 ; c1ccc(-c2cc(-c3nc(-c4ccncc4)nc(-c4ccncc4)n3)cc(-c3ccccc3)c2-n2c3ccccc3c3c2ccc2c4ccc5c(c6ccccc6n5-c5ccccc5)c4n(-c4ccccc4)c23)cc1]

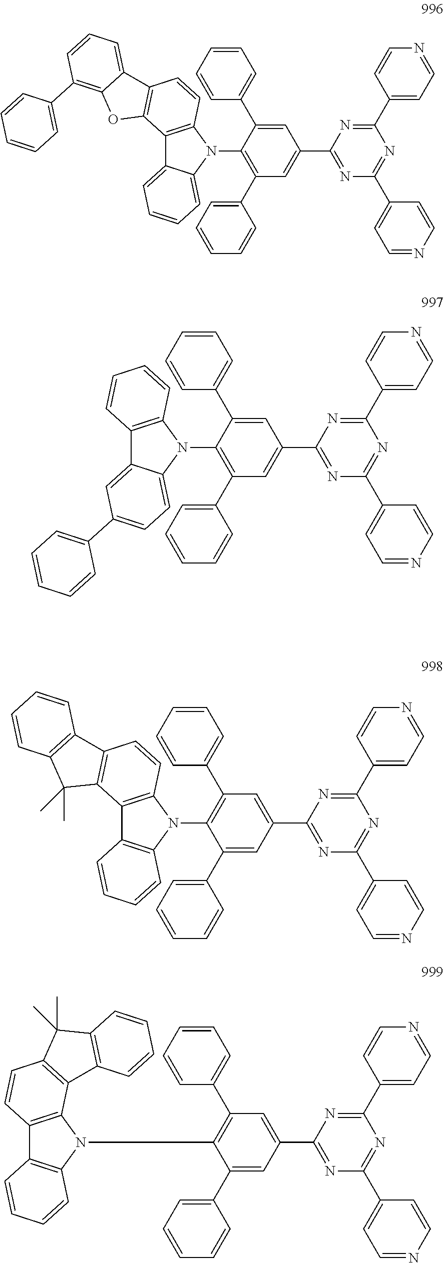 [SMILES: c1ccc(-c2cc(-c3nc(-c4ccncc4)nc(-c4ccncc4)n3)cc(-c3ccccc3)c2-n2c3ccccc3c3c4oc5c(-c6ccccc6)cccc5c4ccc32)cc1 ; c1ccc(-c2ccc3c(c2)c2ccccc2n3-c2c(-c3ccccc3)cc(-c3nc(-c4ccncc4)nc(-c4ccncc4)n3)cc2-c2ccccc2)cc1 ; CC1(C)c2ccccc2-c2ccc3c(c21)c1ccccc1n3-c1c(-c2ccccc2)cc(-c2nc(-c3ccncc3)nc(-c3ccncc3)n2)cc1-c1ccccc1 ; CC1(C)c2ccccc2-c2c1ccc1c3ccccc3n(-c3c(-c4ccccc4)cc(-c4nc(-c5ccncc5)nc(-c5ccncc5)n4)cc3-c3ccccc3)c21]

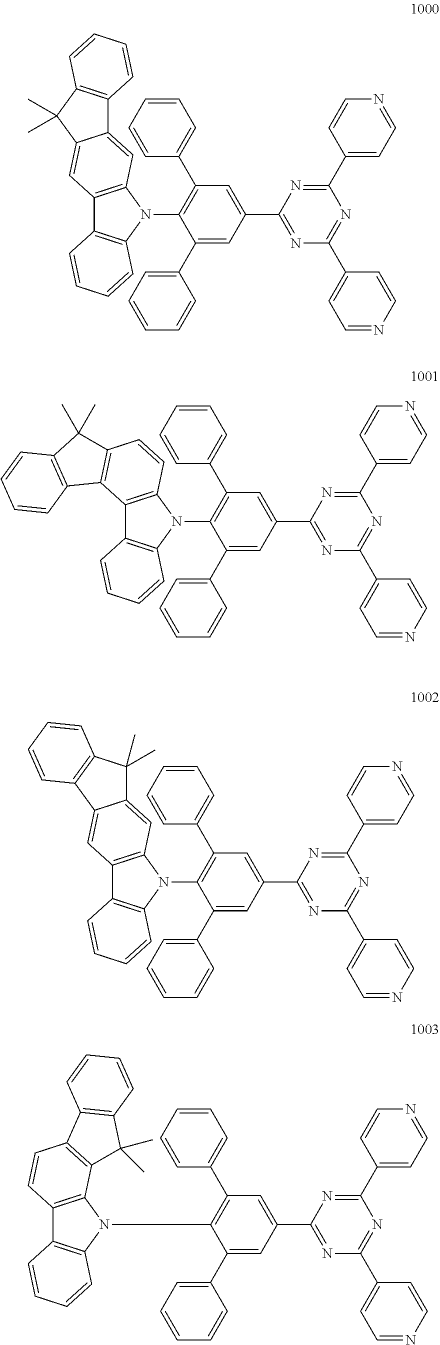 [SMILES: CC1(C)c2ccccc2-c2cc3c(cc21)c1ccccc1n3-c1c(-c2ccccc2)cc(-c2nc(-c3ccncc3)nc(-c3ccncc3)n2)cc1-c1ccccc1 ; CC1(C)c2ccccc2-c2c1ccc1c2c2ccccc2n1-c1c(-c2ccccc2)cc(-c2nc(-c3ccncc3)nc(-c3ccncc3)n2)cc1-c1ccccc1 ; CC1(C)c2ccccc2-c2cc3c4ccccc4n(-c4c(-c5ccccc5)cc(-c5nc(-c6ccncc6)nc(-c6ccncc6)n5)cc4-c4ccccc4)c3cc21 ; CC1(C)c2ccccc2-c2ccc3c4ccccc4n(-c4c(-c5ccccc5)cc(-c5nc(-c6ccncc6)nc(-c6ccncc6)n5)cc4-c4ccccc4)c3c21]

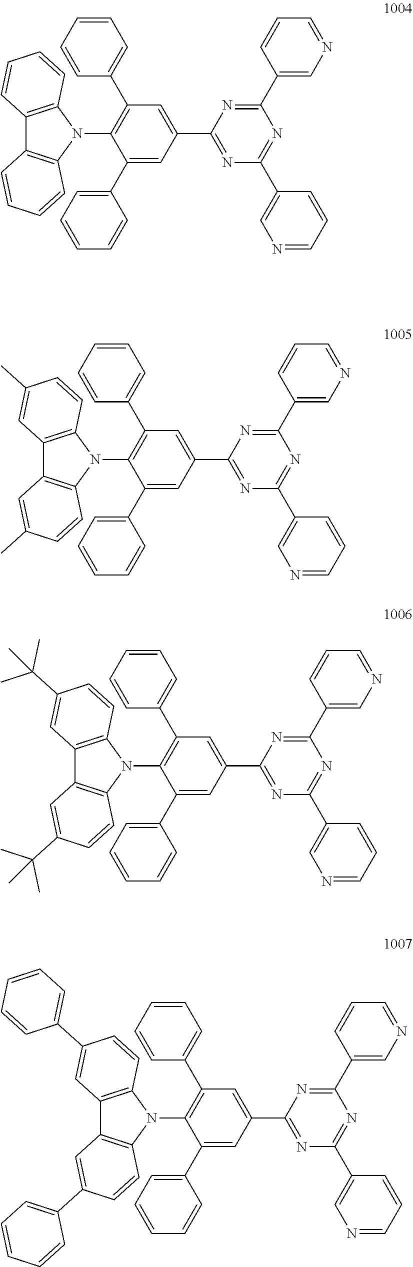 [SMILES: c1ccc(-c2cc(-c3nc(-c4cccnc4)nc(-c4cccnc4)n3)cc(-c3ccccc3)c2-n2c3ccccc3c3ccccc32)cc1 ; Cc1ccc2c(c1)c1cc(C)ccc1n2-c1c(-c2ccccc2)cc(-c2nc(-c3cccnc3)nc(-c3cccnc3)n2)cc1-c1ccccc1 ; CC(C)(C)c1ccc2c(c1)c1cc(C(C)(C)C)ccc1n2-c1c(-c2ccccc2)cc(-c2nc(-c3cccnc3)nc(-c3cccnc3)n2)cc1-c1ccccc1 ; c1ccc(-c2ccc3c(c2)c2cc(-c4ccccc4)ccc2n3-c2c(-c3ccccc3)cc(-c3nc(-c4cccnc4)nc(-c4cccnc4)n3)cc2-c2ccccc2)cc1]

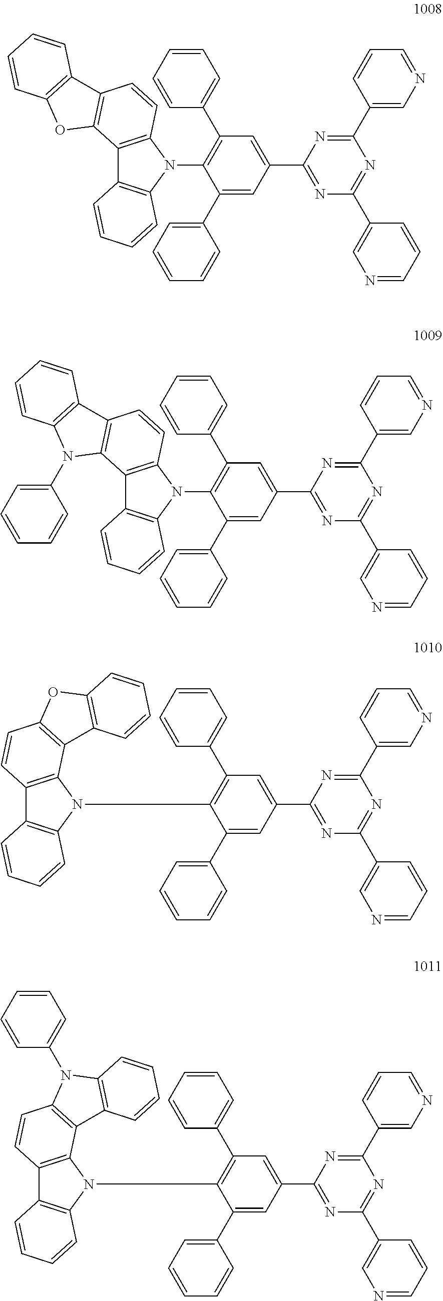 [SMILES: c1ccc(-c2cc(-c3nc(-c4cccnc4)nc(-c4cccnc4)n3)cc(-c3ccccc3)c2-n2c3ccccc3c3c4oc5ccccc5c4ccc32)cc1 ; c1ccc(-c2cc(-c3nc(-c4cccnc4)nc(-c4cccnc4)n3)cc(-c3ccccc3)c2-n2c3ccccc3c3c2ccc2c4ccccc4n(-c4ccccc4)c23)cc1 ; c1ccc(-c2cc(-c3nc(-c4cccnc4)nc(-c4cccnc4)n3)cc(-c3ccccc3)c2-n2c3ccccc3c3ccc4oc5ccccc5c4c32)cc1 ; c1ccc(-c2cc(-c3nc(-c4cccnc4)nc(-c4cccnc4)n3)cc(-c3ccccc3)c2-n2c3ccccc3c3ccc4c(c5ccccc5n4-c4ccccc4)c32)cc1]

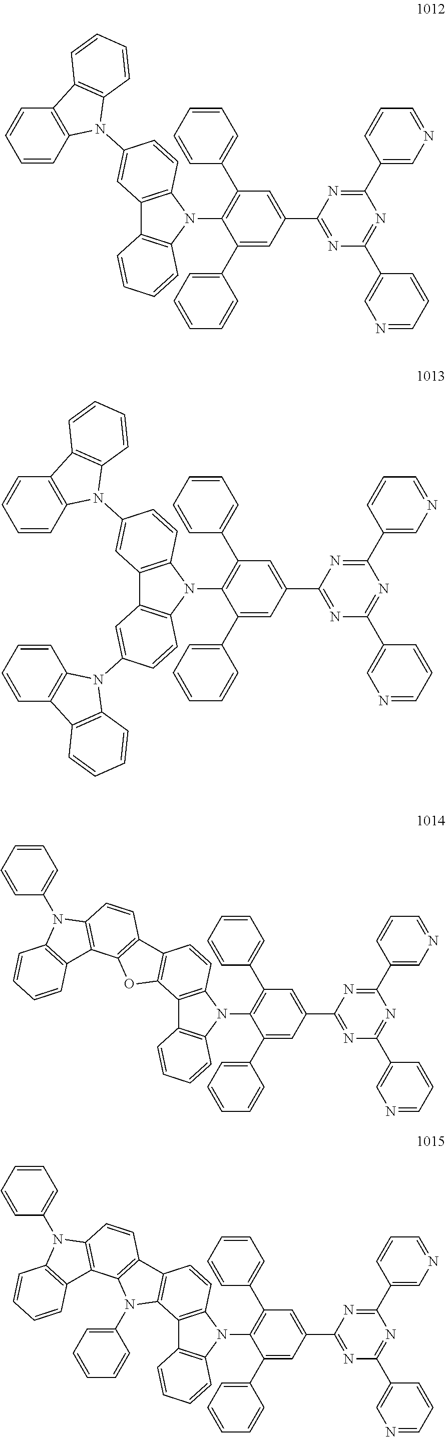 [SMILES: c1ccc(-c2cc(-c3nc(-c4cccnc4)nc(-c4cccnc4)n3)cc(-c3ccccc3)c2-n2c3ccccc3c3cc(-n4c5ccccc5c5ccccc54)ccc32)cc1 ; c1ccc(-c2cc(-c3nc(-c4cccnc4)nc(-c4cccnc4)n3)cc(-c3ccccc3)c2-n2c3ccc(-n4c5ccccc5c5ccccc54)cc3c3cc(-n4c5ccccc5c5ccccc54)ccc32)cc1 ; c1ccc(-c2cc(-c3nc(-c4cccnc4)nc(-c4cccnc4)n3)cc(-c3ccccc3)c2-n2c3ccccc3c3c4oc5c(ccc6c5c5ccccc5n6-c5ccccc5)c4ccc32)cc1 ; c1ccc(-c2cc(-c3nc(-c4cccnc4)nc(-c4cccnc4)n3)cc(-c3ccccc3)c2-n2c3ccccc3c3c2ccc2c4ccc5c(c6ccccc6n5-c5ccccc5)c4n(-c4ccccc4)c23)cc1]

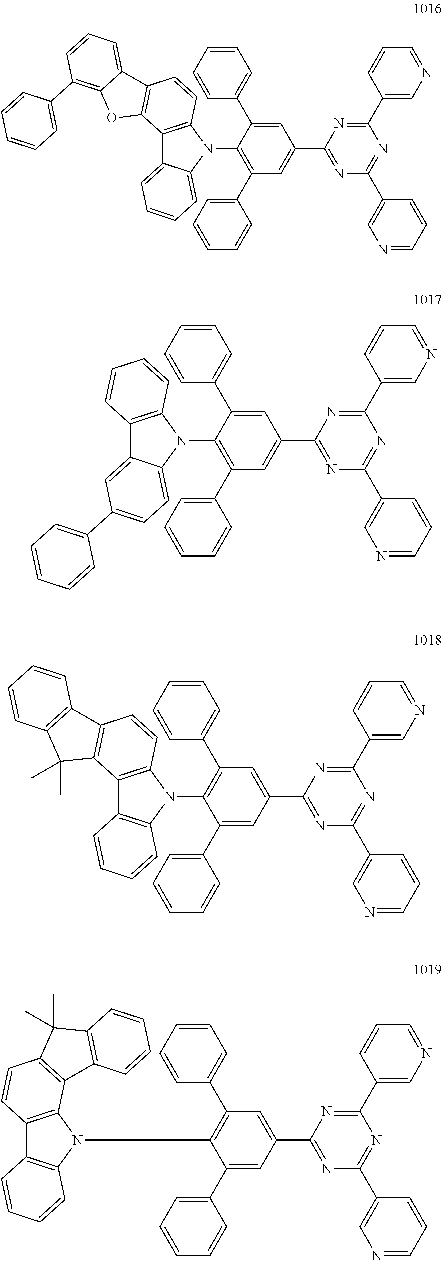 [SMILES: c1ccc(-c2cc(-c3nc(-c4cccnc4)nc(-c4cccnc4)n3)cc(-c3ccccc3)c2-n2c3ccccc3c3c4oc5c(-c6ccccc6)cccc5c4ccc32)cc1 ; c1ccc(-c2ccc3c(c2)c2ccccc2n3-c2c(-c3ccccc3)cc(-c3nc(-c4cccnc4)nc(-c4cccnc4)n3)cc2-c2ccccc2)cc1 ; CC1(C)c2ccccc2-c2ccc3c(c21)c1ccccc1n3-c1c(-c2ccccc2)cc(-c2nc(-c3cccnc3)nc(-c3cccnc3)n2)cc1-c1ccccc1 ; CC1(C)c2ccccc2-c2c1ccc1c3ccccc3n(-c3c(-c4ccccc4)cc(-c4nc(-c5cccnc5)nc(-c5cccnc5)n4)cc3-c3ccccc3)c21]

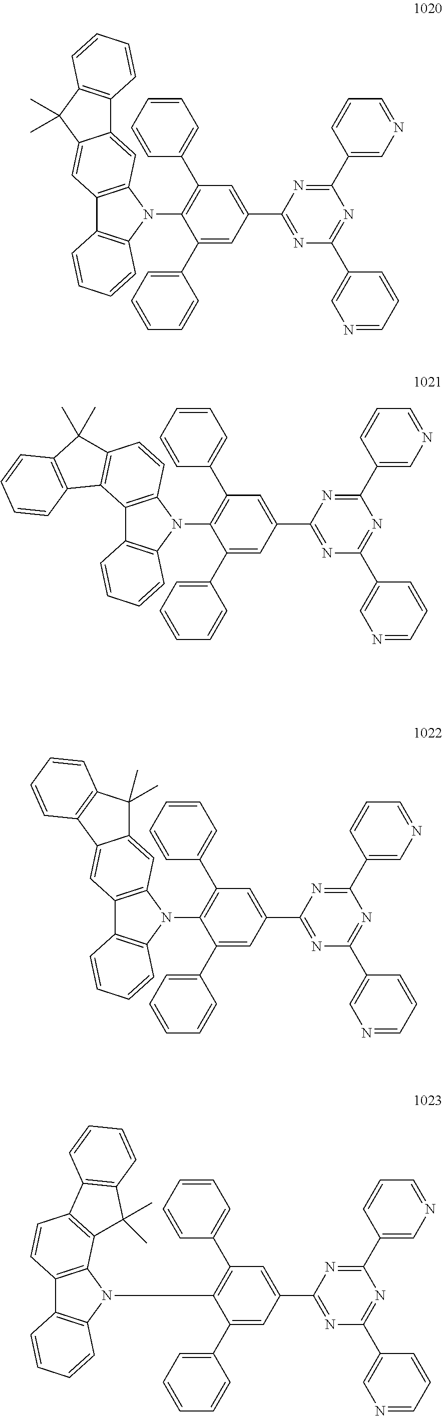 [SMILES: CC1(C)c2ccccc2-c2cc3c(cc21)c1ccccc1n3-c1c(-c2ccccc2)cc(-c2nc(-c3cccnc3)nc(-c3cccnc3)n2)cc1-c1ccccc1 ; CC1(C)c2ccccc2-c2c1ccc1c2c2ccccc2n1-c1c(-c2ccccc2)cc(-c2nc(-c3cccnc3)nc(-c3cccnc3)n2)cc1-c1ccccc1 ; CC1(C)c2ccccc2-c2cc3c4ccccc4n(-c4c(-c5ccccc5)cc(-c5nc(-c6cccnc6)nc(-c6cccnc6)n5)cc4-c4ccccc4)c3cc21 ; CC1(C)c2ccccc2-c2ccc3c4ccccc4n(-c4c(-c5ccccc5)cc(-c5nc(-c6cccnc6)nc(-c6cccnc6)n5)cc4-c4ccccc4)c3c21]

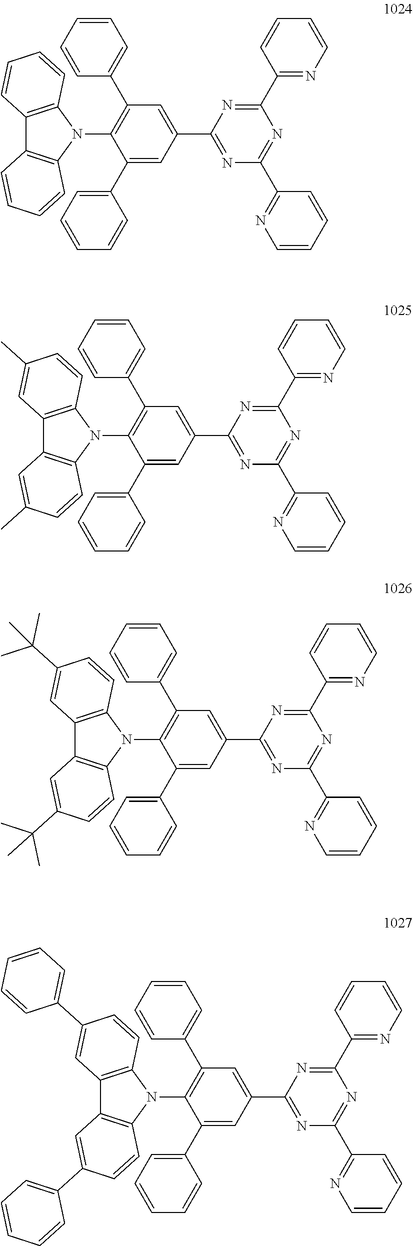 [SMILES: c1ccc(-c2cc(-c3nc(-c4ccccn4)nc(-c4ccccn4)n3)cc(-c3ccccc3)c2-n2c3ccccc3c3ccccc32)cc1 ; Cc1ccc2c(c1)c1cc(C)ccc1n2-c1c(-c2ccccc2)cc(-c2nc(-c3ccccn3)nc(-c3ccccn3)n2)cc1-c1ccccc1 ; CC(C)(C)c1ccc2c(c1)c1cc(C(C)(C)C)ccc1n2-c1c(-c2ccccc2)cc(-c2nc(-c3ccccn3)nc(-c3ccccn3)n2)cc1-c1ccccc1 ; c1ccc(-c2ccc3c(c2)c2cc(-c4ccccc4)ccc2n3-c2c(-c3ccccc3)cc(-c3nc(-c4ccccn4)nc(-c4ccccn4)n3)cc2-c2ccccc2)cc1]

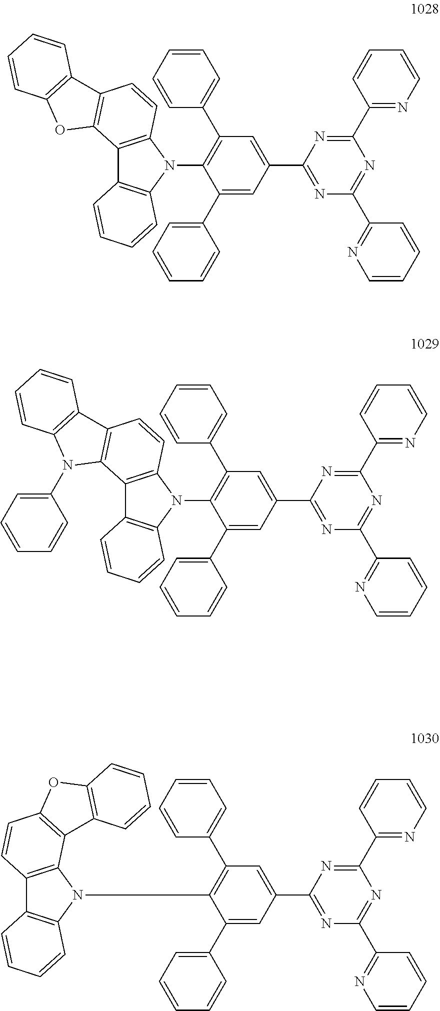 [SMILES: c1ccc(-c2cc(-c3nc(-c4ccccn4)nc(-c4ccccn4)n3)cc(-c3ccccc3)c2-n2c3ccccc3c3c4oc5ccccc5c4ccc32)cc1 ; c1ccc(-c2cc(-c3nc(-c4ccccn4)nc(-c4ccccn4)n3)cc(-c3ccccc3)c2-n2c3ccccc3c3c2ccc2c4ccccc4n(-c4ccccc4)c23)cc1 ; c1ccc(-c2cc(-c3nc(-c4ccccn4)nc(-c4ccccn4)n3)cc(-c3ccccc3)c2-n2c3ccccc3c3ccc4oc5ccccc5c4c32)cc1]

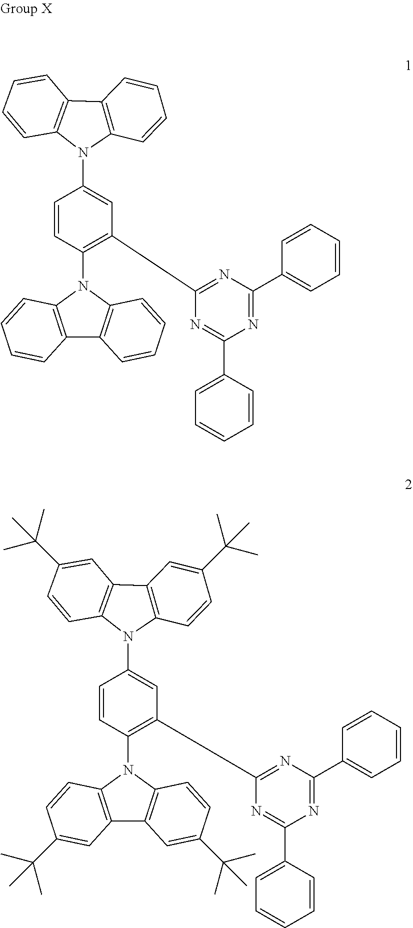 [SMILES: c1ccc(-c2nc(-c3ccccc3)nc(-c3cc(-n4c5ccccc5c5ccccc54)ccc3-n3c4ccccc4c4ccccc43)n2)cc1 ; CC(C)(C)c1ccc2c(c1)c1cc(C(C)(C)C)ccc1n2-c1ccc(-n2c3ccc(C(C)(C)C)cc3c3cc(C(C)(C)C)ccc32)c(-c2nc(-c3ccccc3)nc(-c3ccccc3)n2)c1]

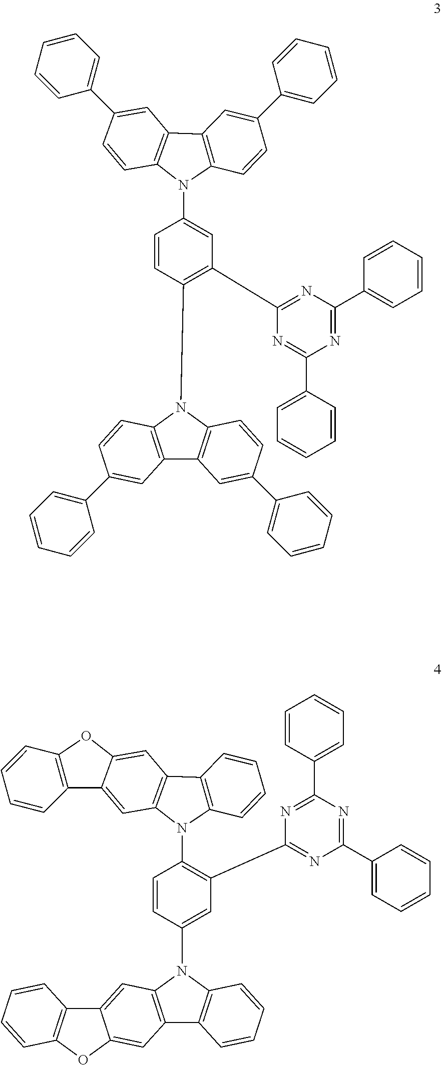 [SMILES: c1ccc(-c2ccc3c(c2)c2cc(-c4ccccc4)ccc2n3-c2ccc(-n3c4ccc(-c5ccccc5)cc4c4cc(-c5ccccc5)ccc43)c(-c3nc(-c4ccccc4)nc(-c4ccccc4)n3)c2)cc1 ; c1ccc(-c2nc(-c3ccccc3)nc(-c3cc(-n4c5ccccc5c5cc6oc7ccccc7c6cc54)ccc3-n3c4ccccc4c4cc5oc6ccccc6c5cc43)n2)cc1]

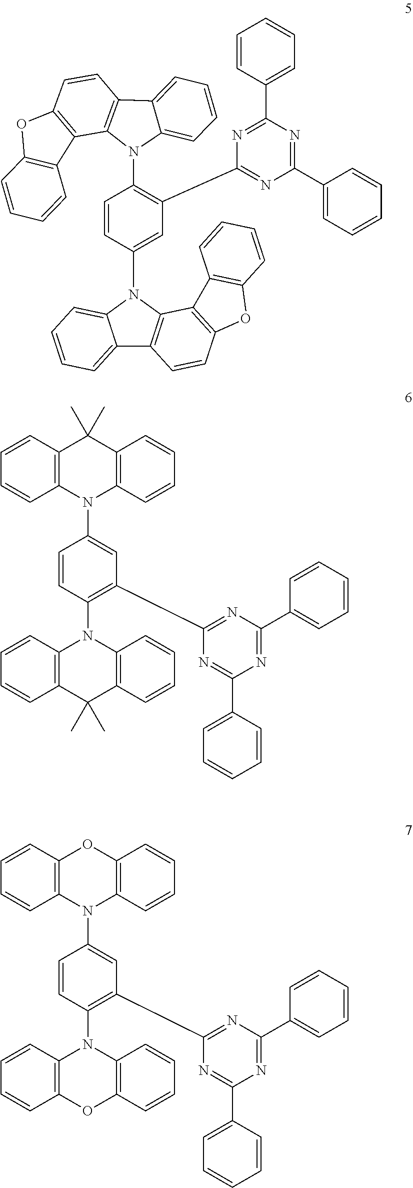

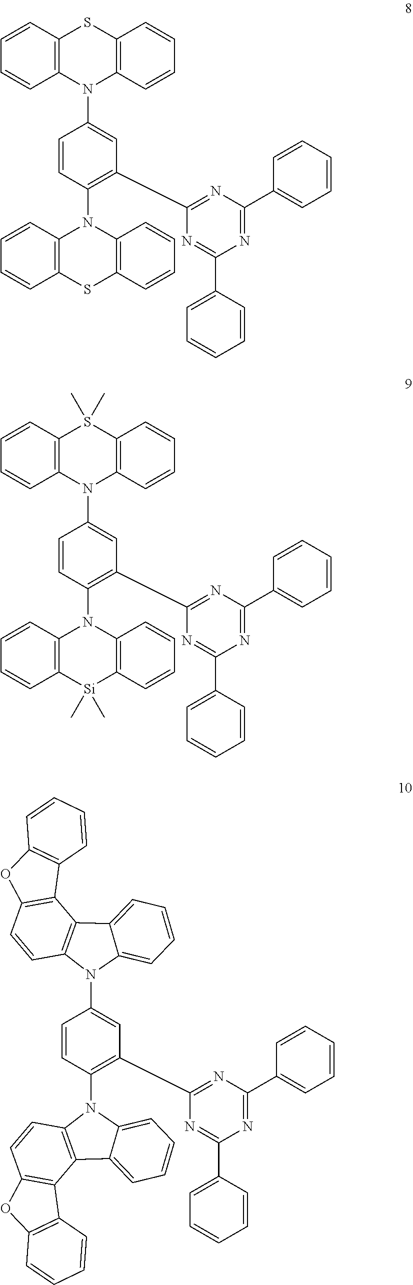 [SMILES: c1ccc(-c2nc(-c3ccccc3)nc(-c3cc(N4c5ccccc5Sc5ccccc54)ccc3N3c4ccccc4Sc4ccccc43)n2)cc1 ; C[Si]1(C)c2ccccc2N(c2ccc(N3c4ccccc4S(C)(C)c4ccccc43)cc2-c2nc(-c3ccccc3)nc(-c3ccccc3)n2)c2ccccc21 ; c1ccc(-c2nc(-c3ccccc3)nc(-c3cc(-n4c5ccccc5c5c6c(ccc54)oc4ccccc46)ccc3-n3c4ccccc4c4c5c(ccc43)oc3ccccc35)n2)cc1]

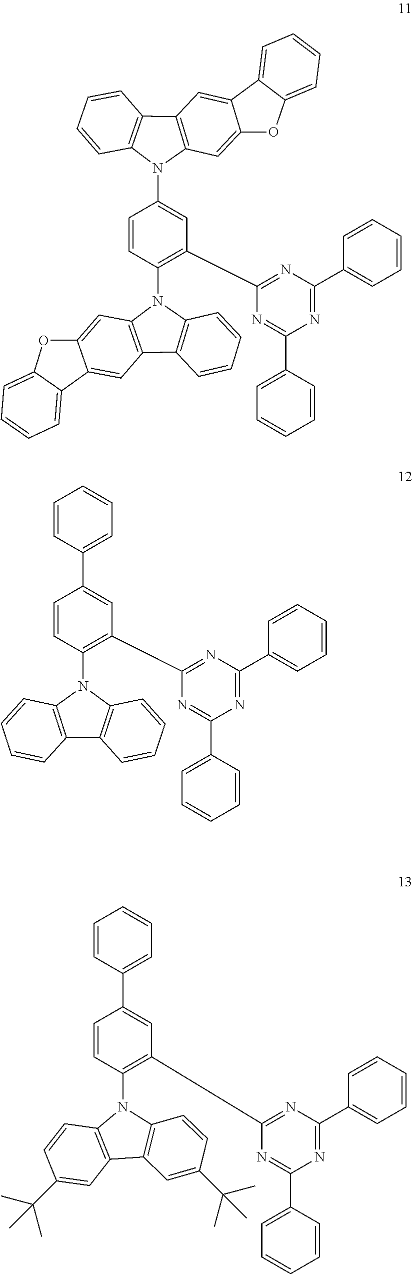 [SMILES: c1ccc(-c2nc(-c3ccccc3)nc(-c3cc(-n4c5ccccc5c5cc6c(cc54)oc4ccccc46)ccc3-n3c4ccccc4c4cc5c(cc43)oc3ccccc35)n2)cc1 ; c1ccc(-c2ccc(-n3c4ccccc4c4ccccc43)c(-c3nc(-c4ccccc4)nc(-c4ccccc4)n3)c2)cc1 ; CC(C)(C)c1ccc2c(c1)c1cc(C(C)(C)C)ccc1n2-c1ccc(-c2ccccc2)cc1-c1nc(-c2ccccc2)nc(-c2ccccc2)n1]

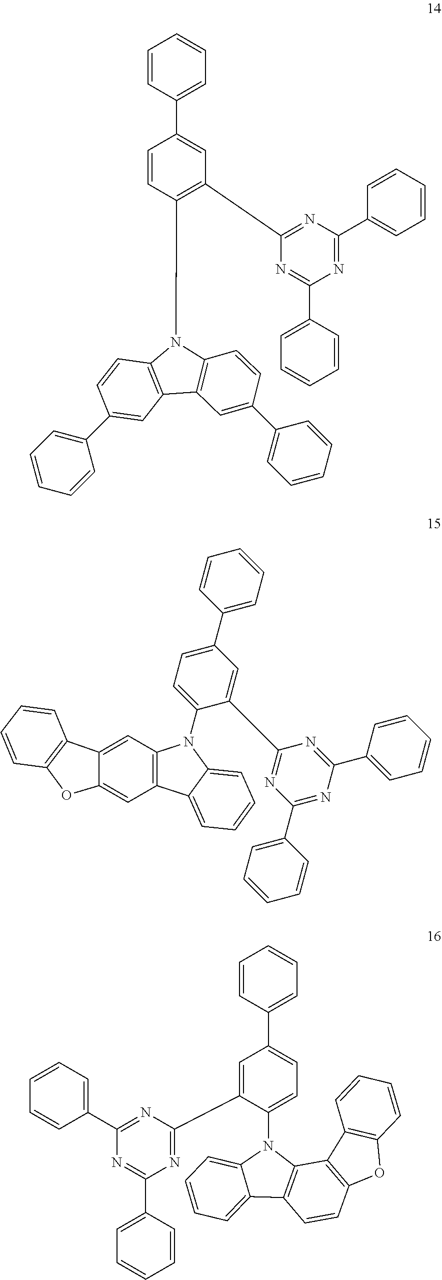

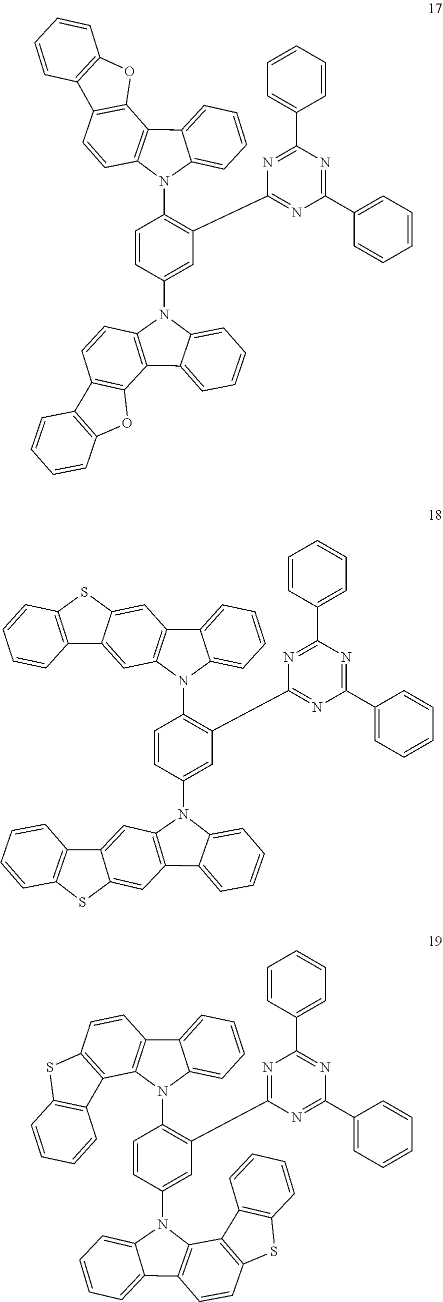 [SMILES: c1ccc(-c2nc(-c3ccccc3)nc(-c3cc(-n4c5ccccc5c5c6oc7ccccc7c6ccc54)ccc3-n3c4ccccc4c4c5oc6ccccc6c5ccc43)n2)cc1 ; c1ccc(-c2nc(-c3ccccc3)nc(-c3cc(-n4c5ccccc5c5cc6sc7ccccc7c6cc54)ccc3-n3c4ccccc4c4cc5sc6ccccc6c5cc43)n2)cc1 ; c1ccc(-c2nc(-c3ccccc3)nc(-c3cc(-n4c5ccccc5c5ccc6sc7ccccc7c6c54)ccc3-n3c4ccccc4c4ccc5sc6ccccc6c5c43)n2)cc1]

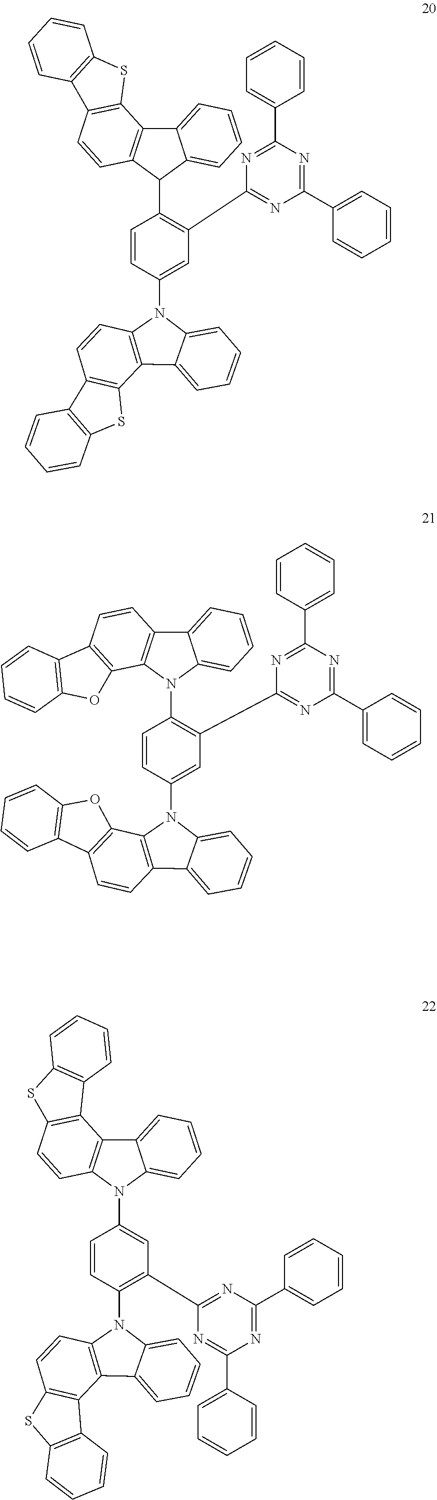 [SMILES: c1ccc(-c2nc(-c3ccccc3)nc(-c3cc(-n4c5ccccc5c5c6sc7ccccc7c6ccc54)ccc3C3c4ccccc4-c4c3ccc3c4sc4ccccc43)n2)cc1 ; c1ccc(-c2nc(-c3ccccc3)nc(-c3cc(-n4c5ccccc5c5ccc6c7ccccc7oc6c54)ccc3-n3c4ccccc4c4ccc5c6ccccc6oc5c43)n2)cc1 ; c1ccc(-c2nc(-c3ccccc3)nc(-c3cc(-n4c5ccccc5c5c6c(ccc54)sc4ccccc46)ccc3-n3c4ccccc4c4c5c(ccc43)sc3ccccc35)n2)cc1]

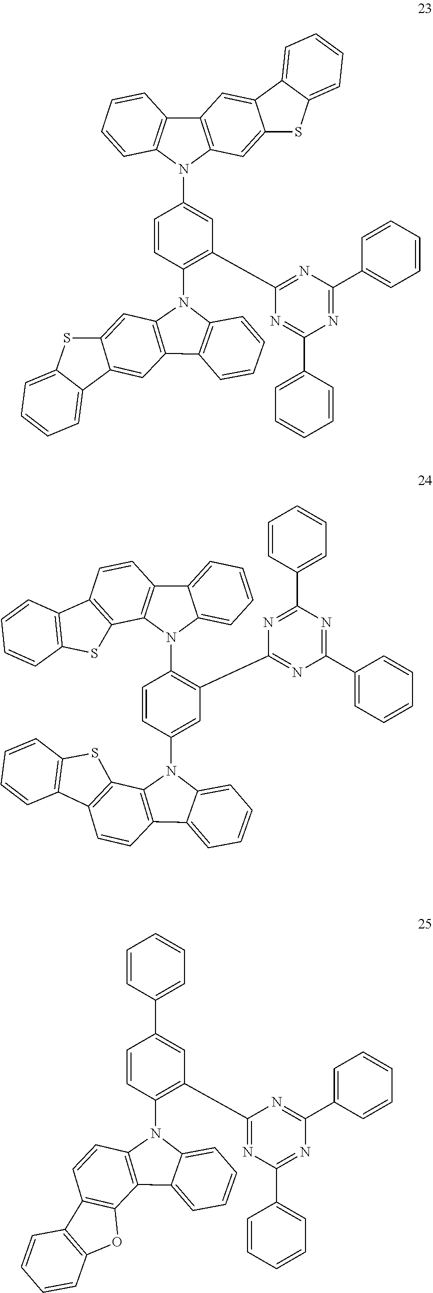 [SMILES: c1ccc(-c2nc(-c3ccccc3)nc(-c3cc(-n4c5ccccc5c5cc6c(cc54)sc4ccccc46)ccc3-n3c4ccccc4c4cc5c(cc43)sc3ccccc35)n2)cc1 ; c1ccc(-c2nc(-c3ccccc3)nc(-c3cc(-n4c5ccccc5c5ccc6c7ccccc7sc6c54)ccc3-n3c4ccccc4c4ccc5c6ccccc6sc5c43)n2)cc1 ; c1ccc(-c2ccc(-n3c4ccccc4c4c5oc6ccccc6c5ccc43)c(-c3nc(-c4ccccc4)nc(-c4ccccc4)n3)c2)cc1]

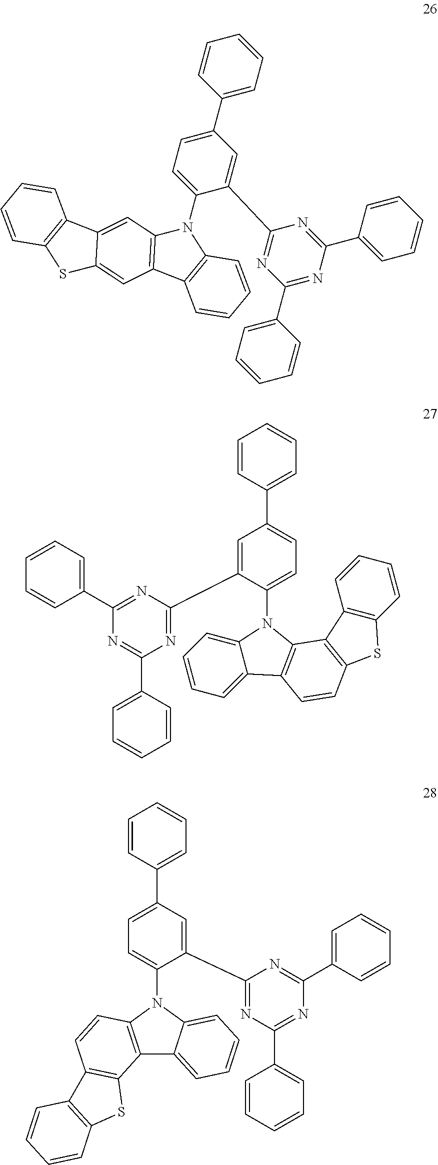

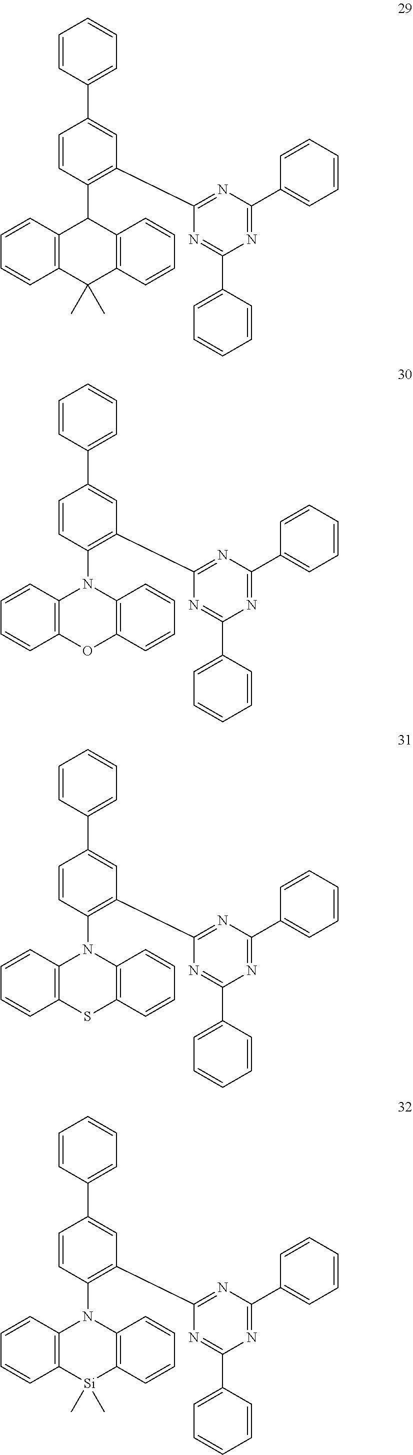 [SMILES: CC1(C)c2ccccc2C(c2ccc(-c3ccccc3)cc2-c2nc(-c3ccccc3)nc(-c3ccccc3)n2)c2ccccc21 ; c1ccc(-c2ccc(N3c4ccccc4Oc4ccccc43)c(-c3nc(-c4ccccc4)nc(-c4ccccc4)n3)c2)cc1 ; c1ccc(-c2ccc(N3c4ccccc4Sc4ccccc43)c(-c3nc(-c4ccccc4)nc(-c4ccccc4)n3)c2)cc1 ; C[Si]1(C)c2ccccc2N(c2ccc(-c3ccccc3)cc2-c2nc(-c3ccccc3)nc(-c3ccccc3)n2)c2ccccc21]

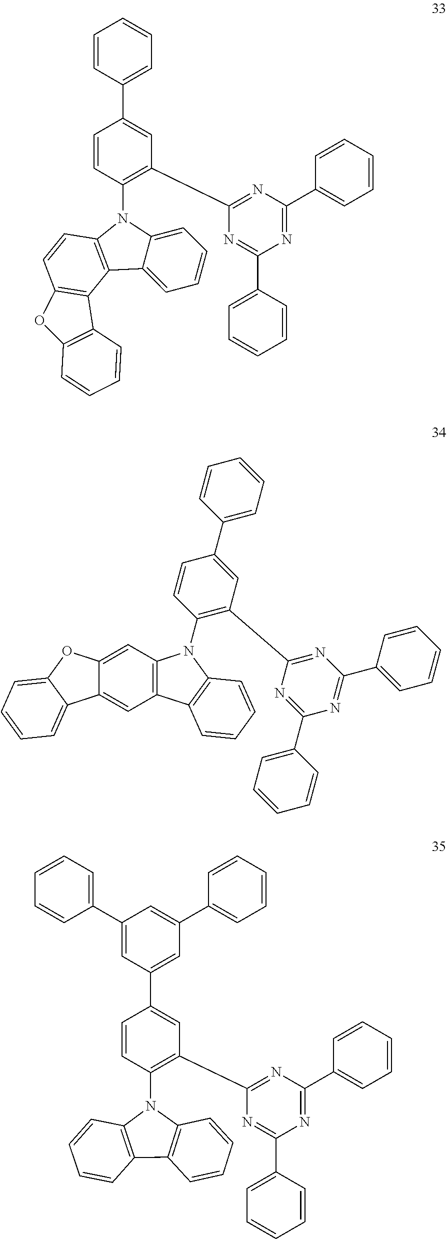 [SMILES: c1ccc(-c2ccc(-n3c4ccccc4c4c5c(ccc43)oc3ccccc35)c(-c3nc(-c4ccccc4)nc(-c4ccccc4)n3)c2)cc1 ; c1ccc(-c2ccc(-n3c4ccccc4c4cc5c(cc43)oc3ccccc35)c(-c3nc(-c4ccccc4)nc(-c4ccccc4)n3)c2)cc1 ; c1ccc(-c2cc(-c3ccccc3)cc(-c3ccc(-n4c5ccccc5c5ccccc54)c(-c4nc(-c5ccccc5)nc(-c5ccccc5)n4)c3)c2)cc1]

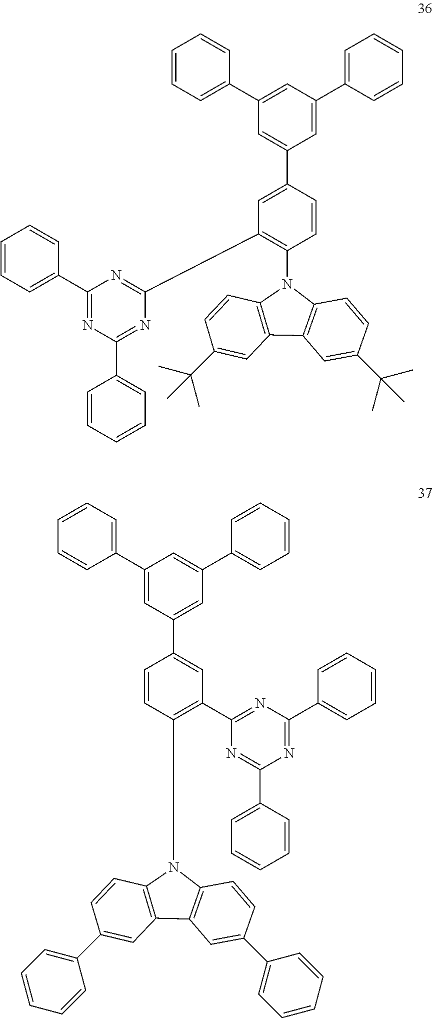 [SMILES: CC(C)(C)c1ccc2c(c1)c1cc(C(C)(C)C)ccc1n2-c1ccc(-c2cc(-c3ccccc3)cc(-c3ccccc3)c2)cc1-c1nc(-c2ccccc2)nc(-c2ccccc2)n1 ; c1ccc(-c2cc(-c3ccccc3)cc(-c3ccc(-n4c5ccc(-c6ccccc6)cc5c5cc(-c6ccccc6)ccc54)c(-c4nc(-c5ccccc5)nc(-c5ccccc5)n4)c3)c2)cc1]

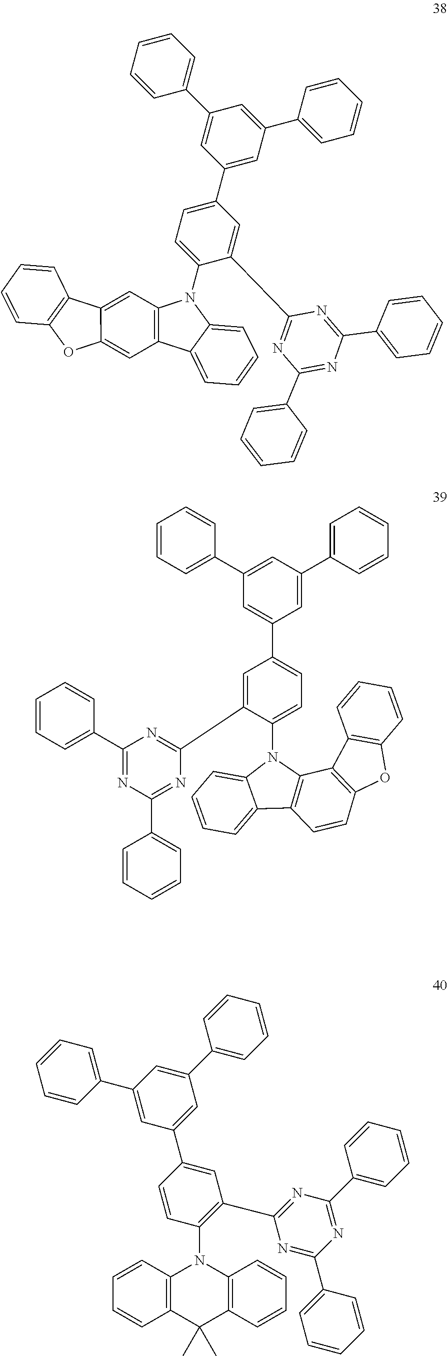 [SMILES: c1ccc(-c2cc(-c3ccccc3)cc(-c3ccc(-n4c5ccccc5c5cc6oc7ccccc7c6cc54)c(-c4nc(-c5ccccc5)nc(-c5ccccc5)n4)c3)c2)cc1 ; c1ccc(-c2cc(-c3ccccc3)cc(-c3ccc(-n4c5ccccc5c5ccc6oc7ccccc7c6c54)c(-c4nc(-c5ccccc5)nc(-c5ccccc5)n4)c3)c2)cc1 ; CC1(C)c2ccccc2N(c2ccc(-c3cc(-c4ccccc4)cc(-c4ccccc4)c3)cc2-c2nc(-c3ccccc3)nc(-c3ccccc3)n2)c2ccccc21]

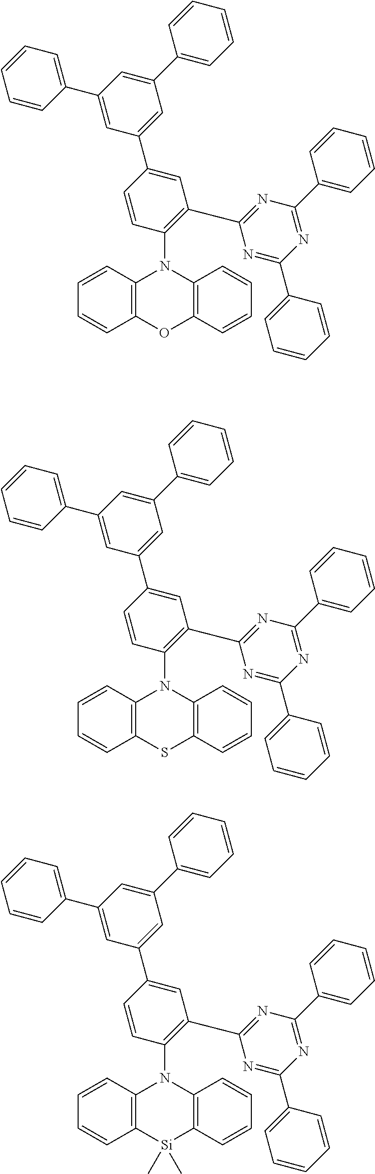 [SMILES: c1ccc(-c2cc(-c3ccccc3)cc(-c3ccc(N4c5ccccc5Oc5ccccc54)c(-c4nc(-c5ccccc5)nc(-c5ccccc5)n4)c3)c2)cc1 ; c1ccc(-c2cc(-c3ccccc3)cc(-c3ccc(N4c5ccccc5Sc5ccccc54)c(-c4nc(-c5ccccc5)nc(-c5ccccc5)n4)c3)c2)cc1 ; C[Si]1(C)c2ccccc2N(c2ccc(-c3cc(-c4ccccc4)cc(-c4ccccc4)c3)cc2-c2nc(-c3ccccc3)nc(-c3ccccc3)n2)c2ccccc21]

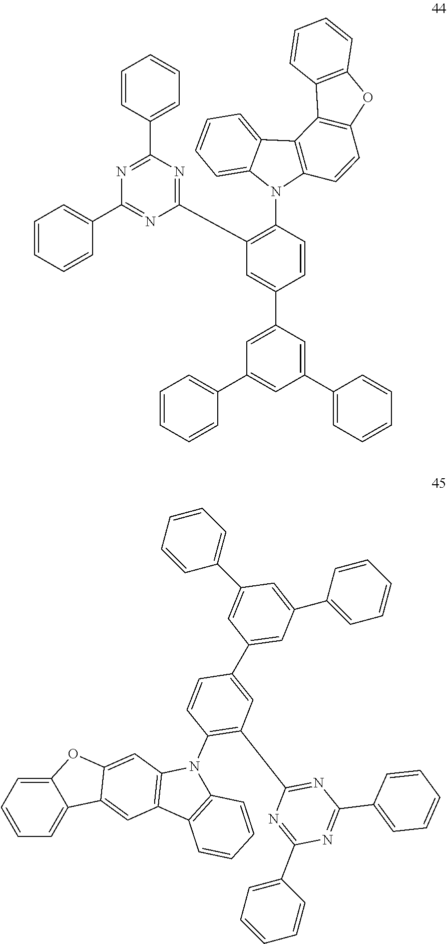 [SMILES: c1ccc(-c2cc(-c3ccccc3)cc(-c3ccc(-n4c5ccccc5c5c6c(ccc54)oc4ccccc46)c(-c4nc(-c5ccccc5)nc(-c5ccccc5)n4)c3)c2)cc1 ; c1ccc(-c2cc(-c3ccccc3)cc(-c3ccc(-n4c5ccccc5c5cc6c(cc54)oc4ccccc46)c(-c4nc(-c5ccccc5)nc(-c5ccccc5)n4)c3)c2)cc1]

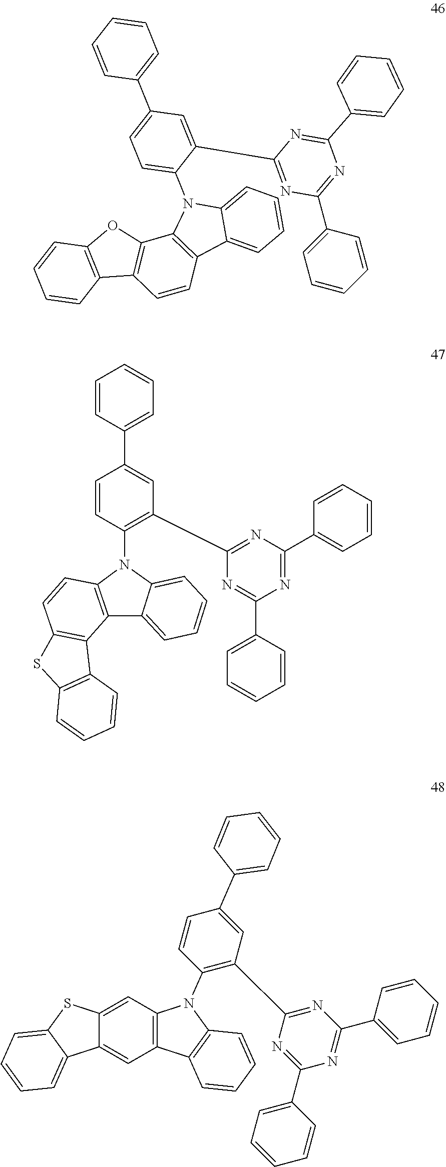 [SMILES: c1ccc(-c2ccc(-n3c4ccccc4c4ccc5c6ccccc6oc5c43)c(-c3nc(-c4ccccc4)nc(-c4ccccc4)n3)c2)cc1 ; c1ccc(-c2ccc(-n3c4ccccc4c4c5c(ccc43)sc3ccccc35)c(-c3nc(-c4ccccc4)nc(-c4ccccc4)n3)c2)cc1 ; c1ccc(-c2ccc(-n3c4ccccc4c4cc5c(cc43)sc3ccccc35)c(-c3nc(-c4ccccc4)nc(-c4ccccc4)n3)c2)cc1]

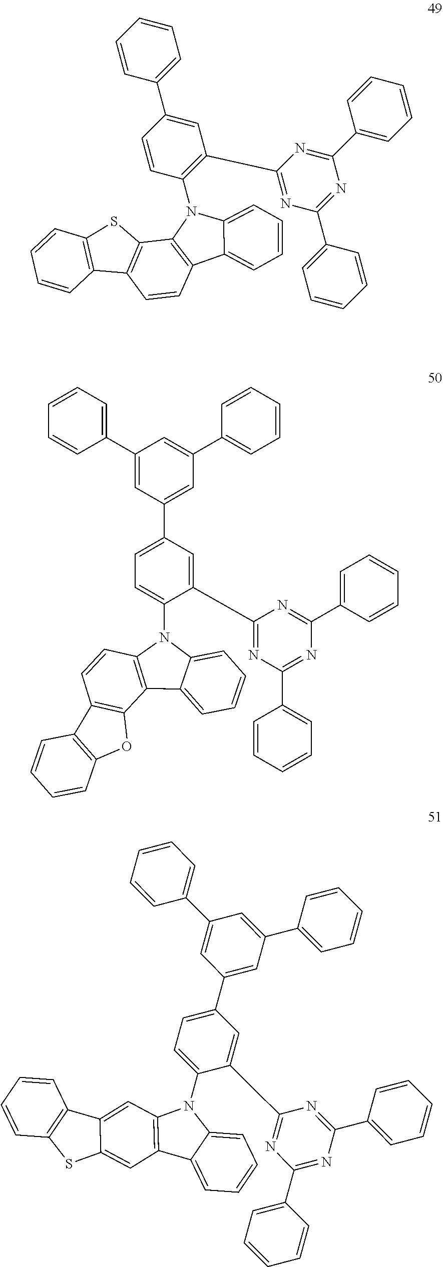 [SMILES: c1ccc(-c2ccc(-n3c4ccccc4c4ccc5c6ccccc6sc5c43)c(-c3nc(-c4ccccc4)nc(-c4ccccc4)n3)c2)cc1 ; c1ccc(-c2cc(-c3ccccc3)cc(-c3ccc(-n4c5ccccc5c5c6oc7ccccc7c6ccc54)c(-c4nc(-c5ccccc5)nc(-c5ccccc5)n4)c3)c2)cc1 ; c1ccc(-c2cc(-c3ccccc3)cc(-c3ccc(-n4c5ccccc5c5cc6sc7ccccc7c6cc54)c(-c4nc(-c5ccccc5)nc(-c5ccccc5)n4)c3)c2)cc1]

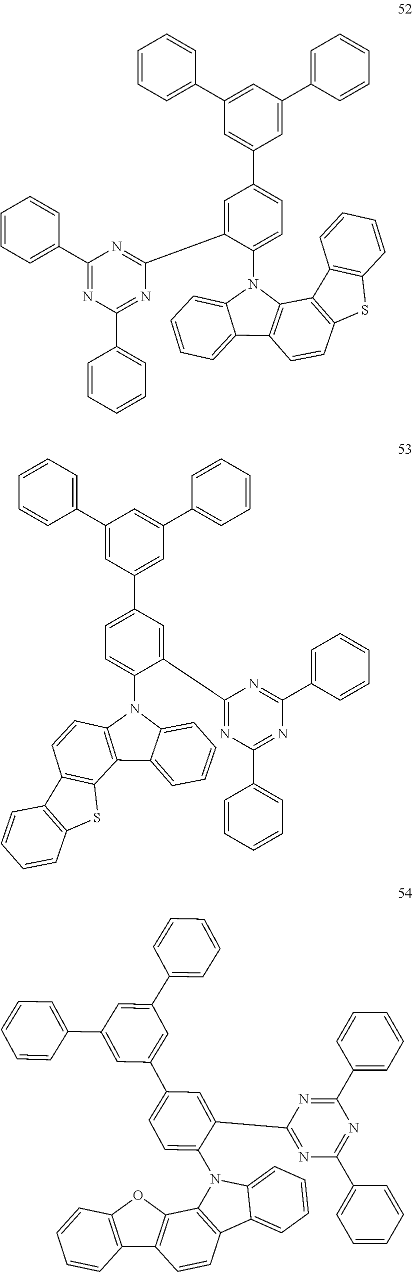 [SMILES: c1ccc(-c2cc(-c3ccccc3)cc(-c3ccc(-n4c5ccccc5c5ccc6sc7ccccc7c6c54)c(-c4nc(-c5ccccc5)nc(-c5ccccc5)n4)c3)c2)cc1 ; c1ccc(-c2cc(-c3ccccc3)cc(-c3ccc(-n4c5ccccc5c5c6sc7ccccc7c6ccc54)c(-c4nc(-c5ccccc5)nc(-c5ccccc5)n4)c3)c2)cc1 ; c1ccc(-c2cc(-c3ccccc3)cc(-c3ccc(-n4c5ccccc5c5ccc6c7ccccc7oc6c54)c(-c4nc(-c5ccccc5)nc(-c5ccccc5)n4)c3)c2)cc1]

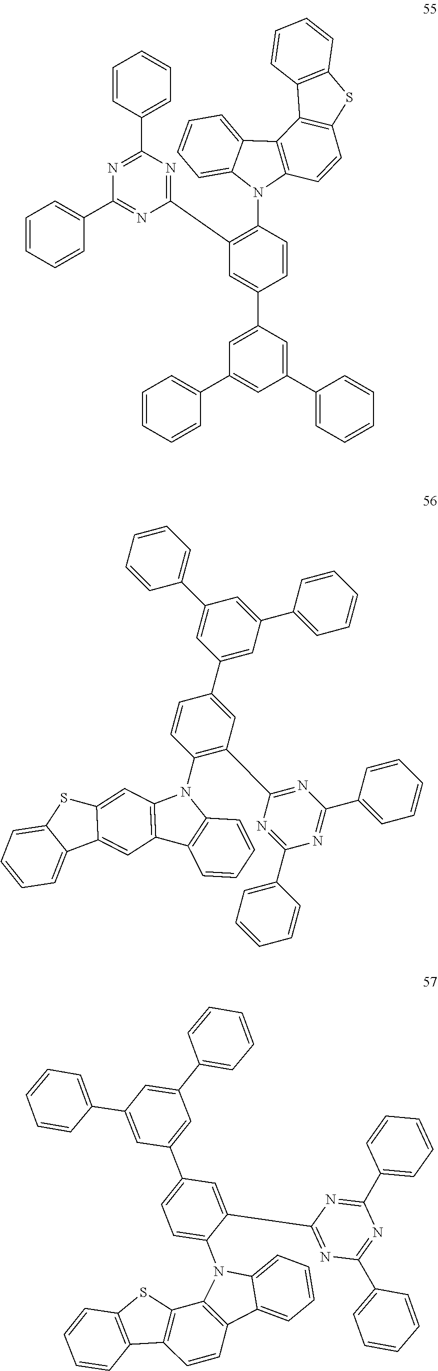 [SMILES: c1ccc(-c2cc(-c3ccccc3)cc(-c3ccc(-n4c5ccccc5c5c6c(ccc54)sc4ccccc46)c(-c4nc(-c5ccccc5)nc(-c5ccccc5)n4)c3)c2)cc1 ; c1ccc(-c2cc(-c3ccccc3)cc(-c3ccc(-n4c5ccccc5c5cc6c(cc54)sc4ccccc46)c(-c4nc(-c5ccccc5)nc(-c5ccccc5)n4)c3)c2)cc1 ; c1ccc(-c2cc(-c3ccccc3)cc(-c3ccc(-n4c5ccccc5c5ccc6c7ccccc7sc6c54)c(-c4nc(-c5ccccc5)nc(-c5ccccc5)n4)c3)c2)cc1]

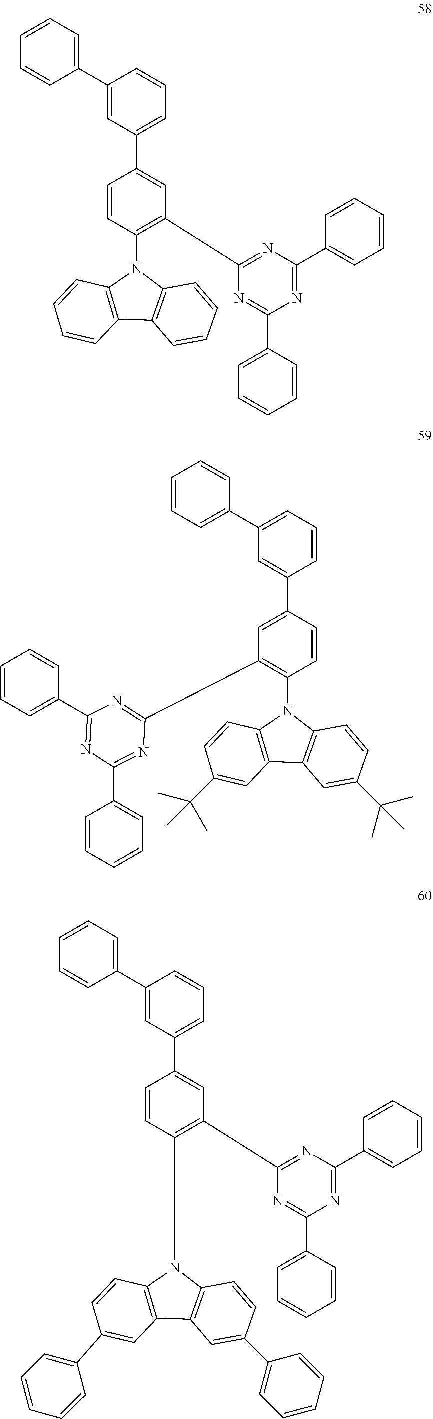 [SMILES: c1ccc(-c2cccc(-c3ccc(-n4c5ccccc5c5ccccc54)c(-c4nc(-c5ccccc5)nc(-c5ccccc5)n4)c3)c2)cc1 ; CC(C)(C)c1ccc2c(c1)c1cc(C(C)(C)C)ccc1n2-c1ccc(-c2cccc(-c3ccccc3)c2)cc1-c1nc(-c2ccccc2)nc(-c2ccccc2)n1 ; c1ccc(-c2cccc(-c3ccc(-n4c5ccc(-c6ccccc6)cc5c5cc(-c6ccccc6)ccc54)c(-c4nc(-c5ccccc5)nc(-c5ccccc5)n4)c3)c2)cc1]

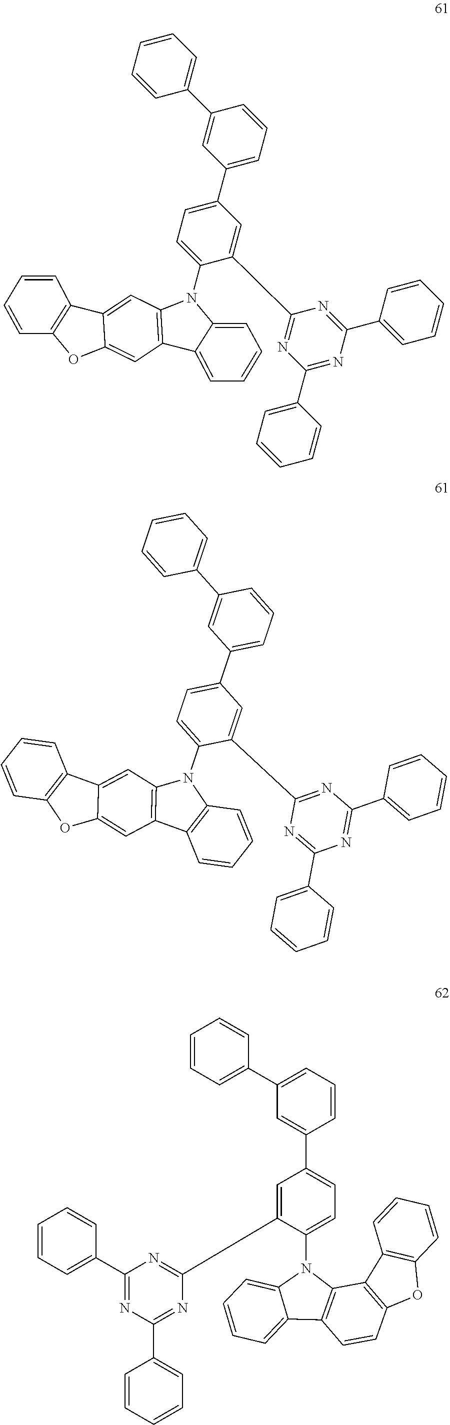 [SMILES: c1ccc(-c2cccc(-c3ccc(-n4c5ccccc5c5cc6oc7ccccc7c6cc54)c(-c4nc(-c5ccccc5)nc(-c5ccccc5)n4)c3)c2)cc1 ; c1ccc(-c2cccc(-c3ccc(-n4c5ccccc5c5cc6oc7ccccc7c6cc54)c(-c4nc(-c5ccccc5)nc(-c5ccccc5)n4)c3)c2)cc1 ; c1ccc(-c2cccc(-c3ccc(-n4c5ccccc5c5ccc6oc7ccccc7c6c54)c(-c4nc(-c5ccccc5)nc(-c5ccccc5)n4)c3)c2)cc1]

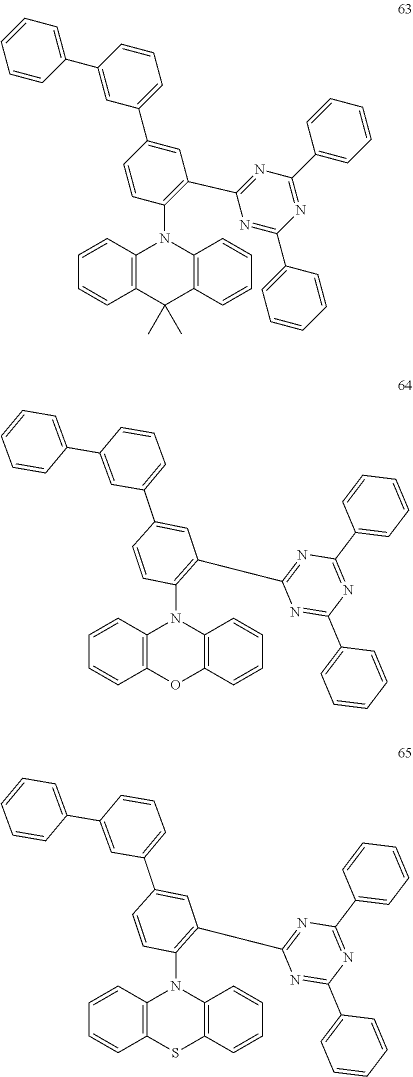 [SMILES: CC1(C)c2ccccc2N(c2ccc(-c3cccc(-c4ccccc4)c3)cc2-c2nc(-c3ccccc3)nc(-c3ccccc3)n2)c2ccccc21 ; c1ccc(-c2cccc(-c3ccc(N4c5ccccc5Oc5ccccc54)c(-c4nc(-c5ccccc5)nc(-c5ccccc5)n4)c3)c2)cc1 ; c1ccc(-c2cccc(-c3ccc(N4c5ccccc5Sc5ccccc54)c(-c4nc(-c5ccccc5)nc(-c5ccccc5)n4)c3)c2)cc1]

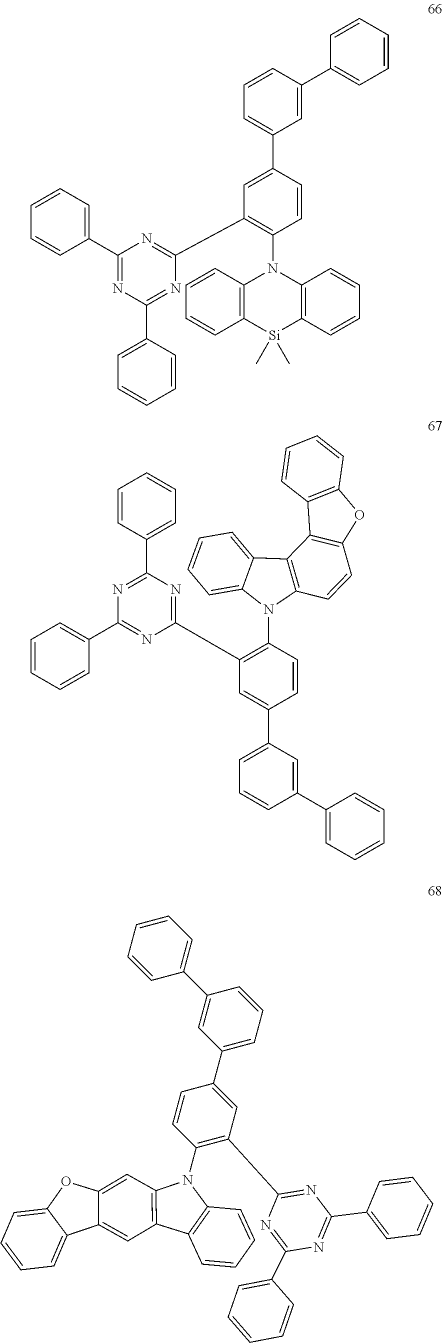 [SMILES: C[Si]1(C)c2ccccc2N(c2ccc(-c3cccc(-c4ccccc4)c3)cc2-c2nc(-c3ccccc3)nc(-c3ccccc3)n2)c2ccccc21 ; c1ccc(-c2cccc(-c3ccc(-n4c5ccccc5c5c6c(ccc54)oc4ccccc46)c(-c4nc(-c5ccccc5)nc(-c5ccccc5)n4)c3)c2)cc1 ; c1ccc(-c2cccc(-c3ccc(-n4c5ccccc5c5cc6c(cc54)oc4ccccc46)c(-c4nc(-c5ccccc5)nc(-c5ccccc5)n4)c3)c2)cc1]

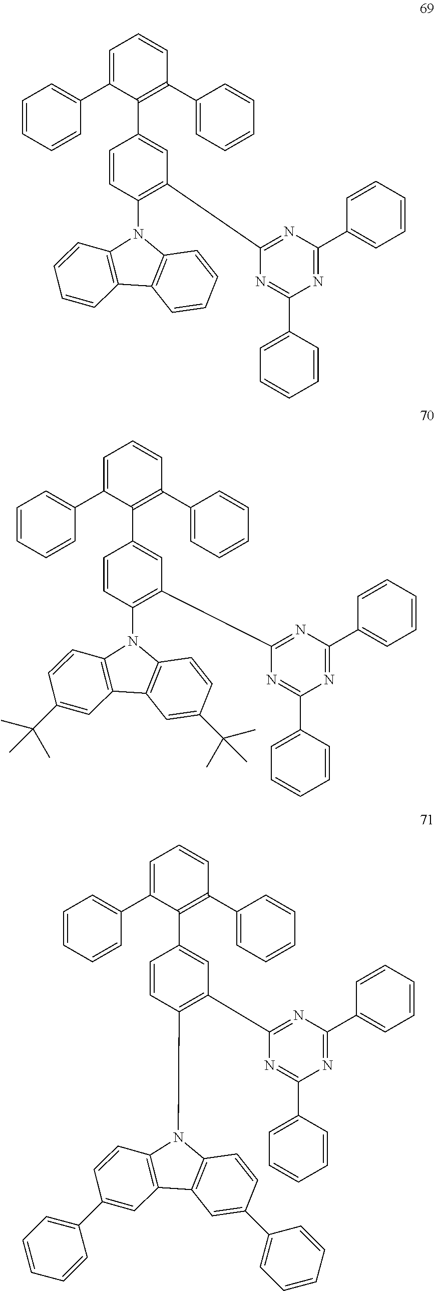 [SMILES: c1ccc(-c2nc(-c3ccccc3)nc(-c3cc(-c4c(-c5ccccc5)cccc4-c4ccccc4)ccc3-n3c4ccccc4c4ccccc43)n2)cc1 ; CC(C)(C)c1ccc2c(c1)c1cc(C(C)(C)C)ccc1n2-c1ccc(-c2c(-c3ccccc3)cccc2-c2ccccc2)cc1-c1nc(-c2ccccc2)nc(-c2ccccc2)n1 ; c1ccc(-c2ccc3c(c2)c2cc(-c4ccccc4)ccc2n3-c2ccc(-c3c(-c4ccccc4)cccc3-c3ccccc3)cc2-c2nc(-c3ccccc3)nc(-c3ccccc3)n2)cc1]

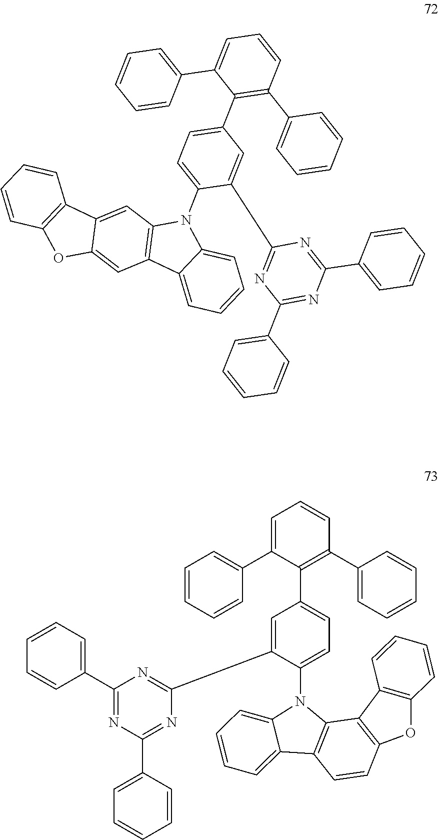 [SMILES: c1ccc(-c2nc(-c3ccccc3)nc(-c3cc(-c4c(-c5ccccc5)cccc4-c4ccccc4)ccc3-n3c4ccccc4c4cc5oc6ccccc6c5cc43)n2)cc1 ; c1ccc(-c2nc(-c3ccccc3)nc(-c3cc(-c4c(-c5ccccc5)cccc4-c4ccccc4)ccc3-n3c4ccccc4c4ccc5oc6ccccc6c5c43)n2)cc1]

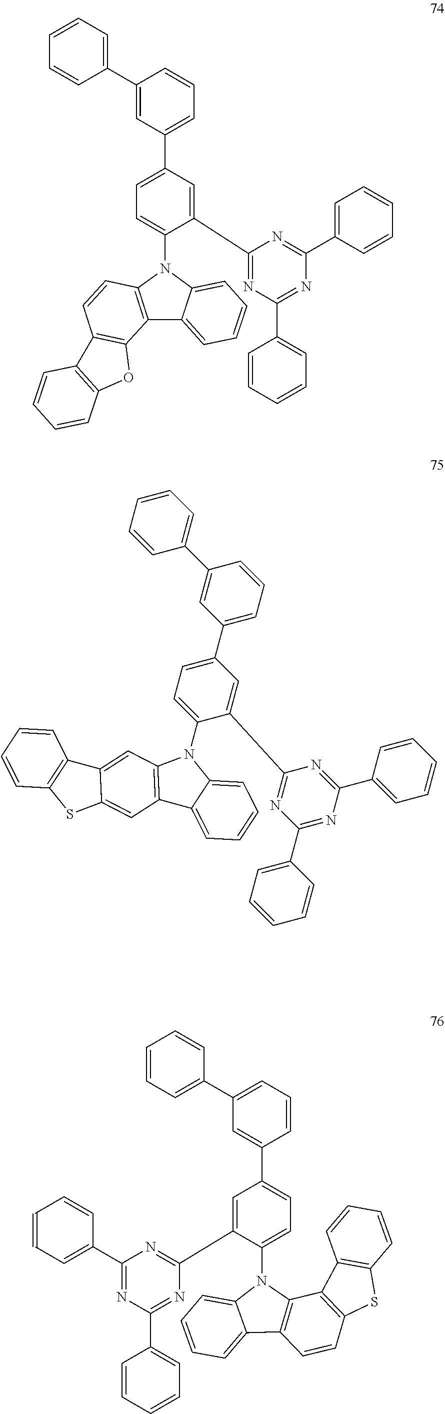 [SMILES: c1ccc(-c2cccc(-c3ccc(-n4c5ccccc5c5c6oc7ccccc7c6ccc54)c(-c4nc(-c5ccccc5)nc(-c5ccccc5)n4)c3)c2)cc1 ; c1ccc(-c2cccc(-c3ccc(-n4c5ccccc5c5cc6sc7ccccc7c6cc54)c(-c4nc(-c5ccccc5)nc(-c5ccccc5)n4)c3)c2)cc1 ; c1ccc(-c2cccc(-c3ccc(-n4c5ccccc5c5ccc6sc7ccccc7c6c54)c(-c4nc(-c5ccccc5)nc(-c5ccccc5)n4)c3)c2)cc1]

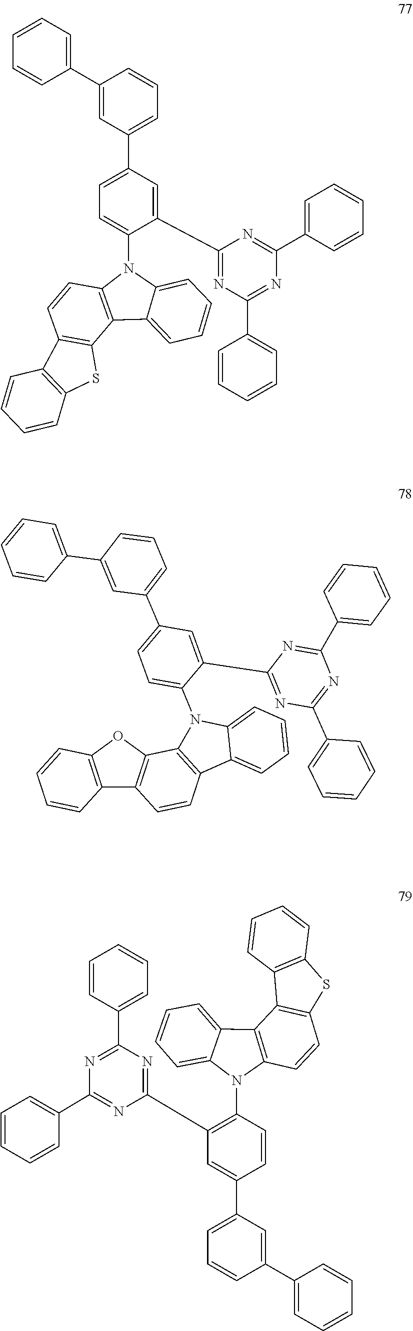 [SMILES: c1ccc(-c2cccc(-c3ccc(-n4c5ccccc5c5c6sc7ccccc7c6ccc54)c(-c4nc(-c5ccccc5)nc(-c5ccccc5)n4)c3)c2)cc1 ; c1ccc(-c2cccc(-c3ccc(-n4c5ccccc5c5ccc6c7ccccc7oc6c54)c(-c4nc(-c5ccccc5)nc(-c5ccccc5)n4)c3)c2)cc1 ; c1ccc(-c2cccc(-c3ccc(-n4c5ccccc5c5c6c(ccc54)sc4ccccc46)c(-c4nc(-c5ccccc5)nc(-c5ccccc5)n4)c3)c2)cc1]

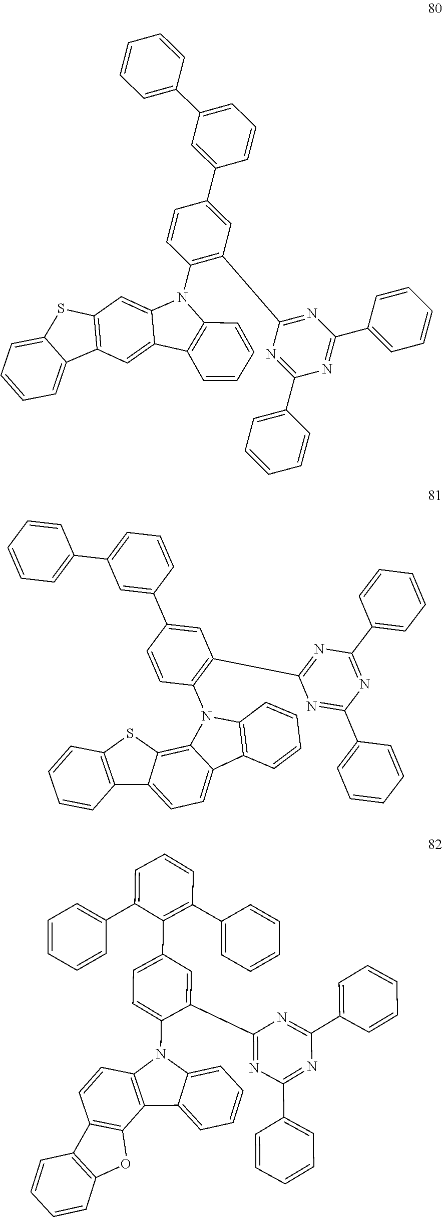

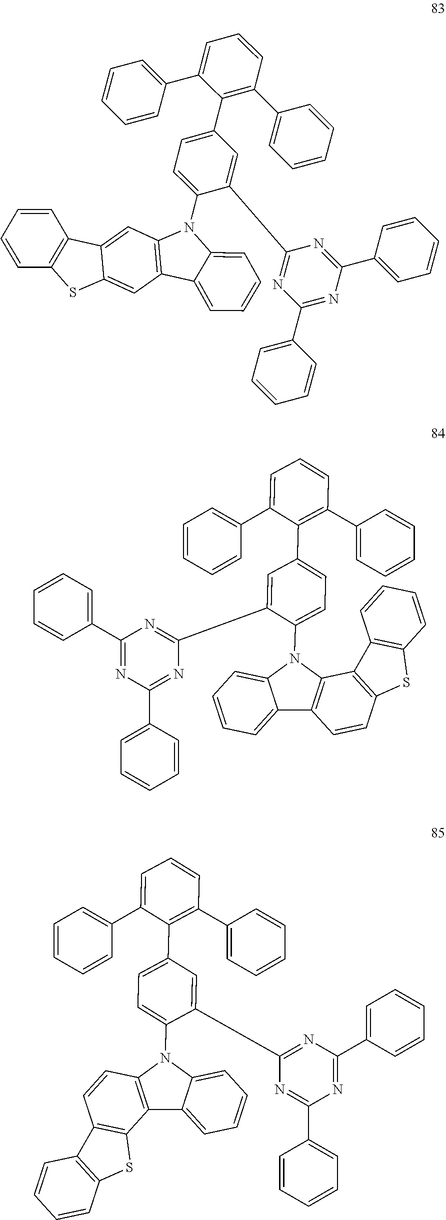 [SMILES: c1ccc(-c2nc(-c3ccccc3)nc(-c3cc(-c4c(-c5ccccc5)cccc4-c4ccccc4)ccc3-n3c4ccccc4c4cc5sc6ccccc6c5cc43)n2)cc1 ; c1ccc(-c2nc(-c3ccccc3)nc(-c3cc(-c4c(-c5ccccc5)cccc4-c4ccccc4)ccc3-n3c4ccccc4c4ccc5sc6ccccc6c5c43)n2)cc1 ; c1ccc(-c2nc(-c3ccccc3)nc(-c3cc(-c4c(-c5ccccc5)cccc4-c4ccccc4)ccc3-n3c4ccccc4c4c5sc6ccccc6c5ccc43)n2)cc1]

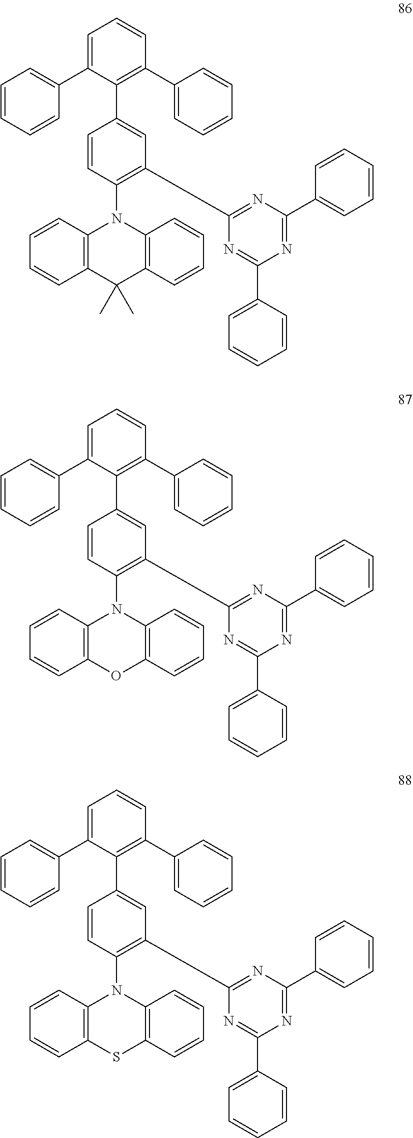 [SMILES: CC1(C)c2ccccc2N(c2ccc(-c3c(-c4ccccc4)cccc3-c3ccccc3)cc2-c2nc(-c3ccccc3)nc(-c3ccccc3)n2)c2ccccc21 ; c1ccc(-c2nc(-c3ccccc3)nc(-c3cc(-c4c(-c5ccccc5)cccc4-c4ccccc4)ccc3N3c4ccccc4Oc4ccccc43)n2)cc1 ; c1ccc(-c2nc(-c3ccccc3)nc(-c3cc(-c4c(-c5ccccc5)cccc4-c4ccccc4)ccc3N3c4ccccc4Sc4ccccc43)n2)cc1]

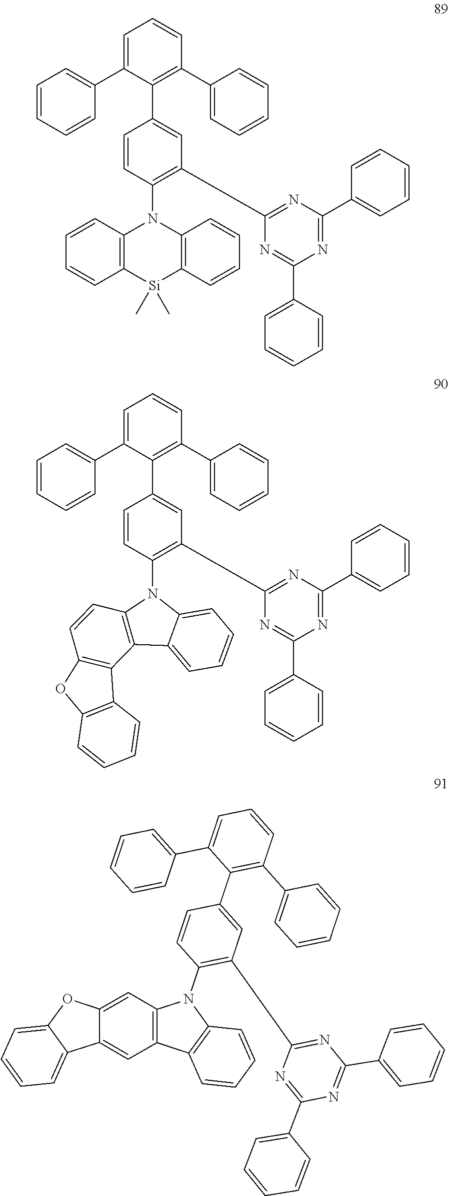 [SMILES: C[Si]1(C)c2ccccc2N(c2ccc(-c3c(-c4ccccc4)cccc3-c3ccccc3)cc2-c2nc(-c3ccccc3)nc(-c3ccccc3)n2)c2ccccc21 ; c1ccc(-c2nc(-c3ccccc3)nc(-c3cc(-c4c(-c5ccccc5)cccc4-c4ccccc4)ccc3-n3c4ccccc4c4c5c(ccc43)oc3ccccc35)n2)cc1 ; c1ccc(-c2nc(-c3ccccc3)nc(-c3cc(-c4c(-c5ccccc5)cccc4-c4ccccc4)ccc3-n3c4ccccc4c4cc5c(cc43)oc3ccccc35)n2)cc1]

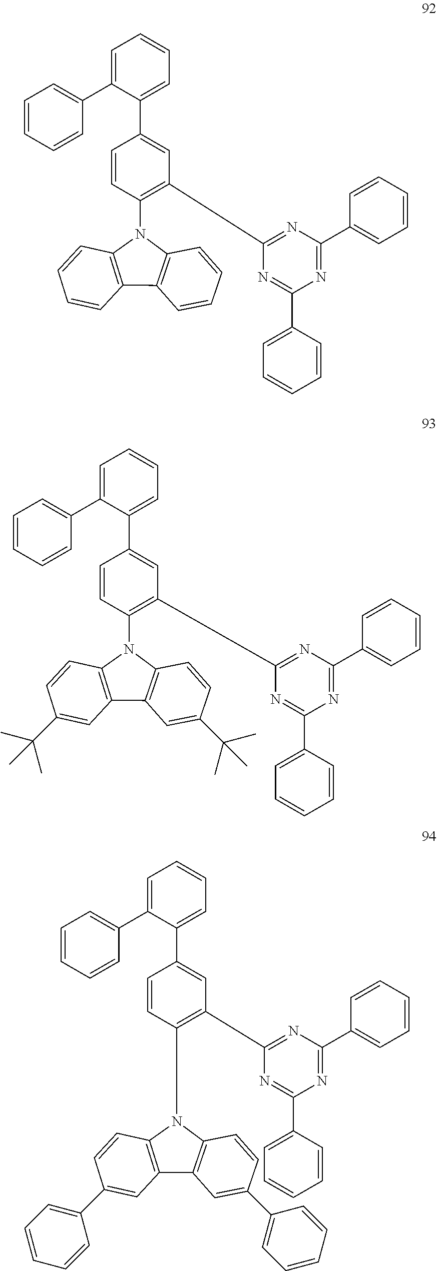 [SMILES: c1ccc(-c2nc(-c3ccccc3)nc(-c3cc(-c4ccccc4-c4ccccc4)ccc3-n3c4ccccc4c4ccccc43)n2)cc1 ; CC(C)(C)c1ccc2c(c1)c1cc(C(C)(C)C)ccc1n2-c1ccc(-c2ccccc2-c2ccccc2)cc1-c1nc(-c2ccccc2)nc(-c2ccccc2)n1 ; c1ccc(-c2ccc3c(c2)c2cc(-c4ccccc4)ccc2n3-c2ccc(-c3ccccc3-c3ccccc3)cc2-c2nc(-c3ccccc3)nc(-c3ccccc3)n2)cc1]

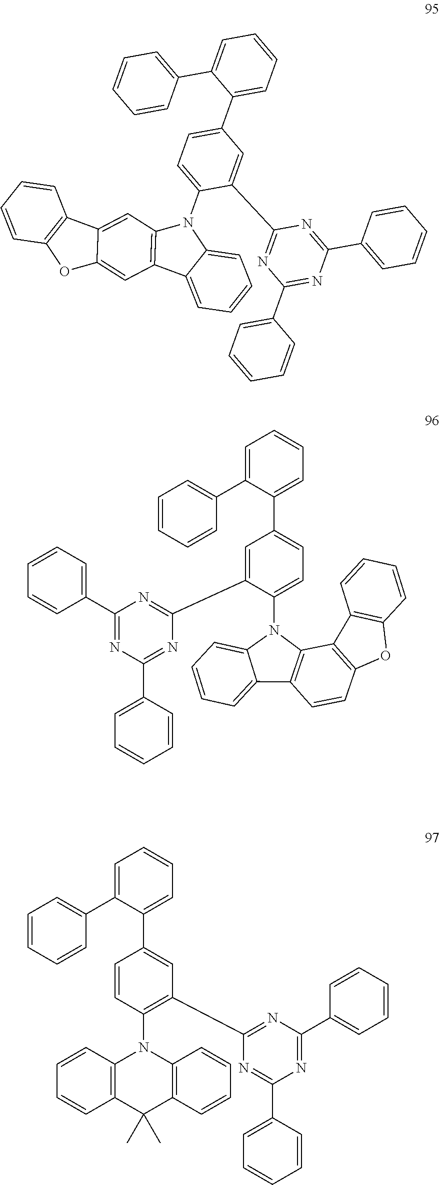 [SMILES: c1ccc(-c2nc(-c3ccccc3)nc(-c3cc(-c4ccccc4-c4ccccc4)ccc3-n3c4ccccc4c4cc5oc6ccccc6c5cc43)n2)cc1 ; c1ccc(-c2nc(-c3ccccc3)nc(-c3cc(-c4ccccc4-c4ccccc4)ccc3-n3c4ccccc4c4ccc5oc6ccccc6c5c43)n2)cc1 ; CC1(C)c2ccccc2N(c2ccc(-c3ccccc3-c3ccccc3)cc2-c2nc(-c3ccccc3)nc(-c3ccccc3)n2)c2ccccc21]

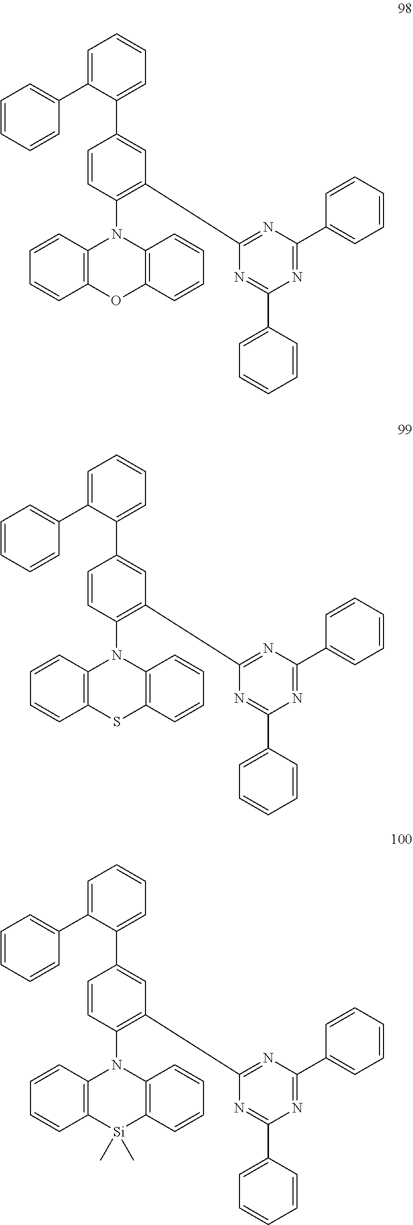 [SMILES: c1ccc(-c2nc(-c3ccccc3)nc(-c3cc(-c4ccccc4-c4ccccc4)ccc3N3c4ccccc4Oc4ccccc43)n2)cc1 ; c1ccc(-c2nc(-c3ccccc3)nc(-c3cc(-c4ccccc4-c4ccccc4)ccc3N3c4ccccc4Sc4ccccc43)n2)cc1 ; C[Si]1(C)c2ccccc2N(c2ccc(-c3ccccc3-c3ccccc3)cc2-c2nc(-c3ccccc3)nc(-c3ccccc3)n2)c2ccccc21]

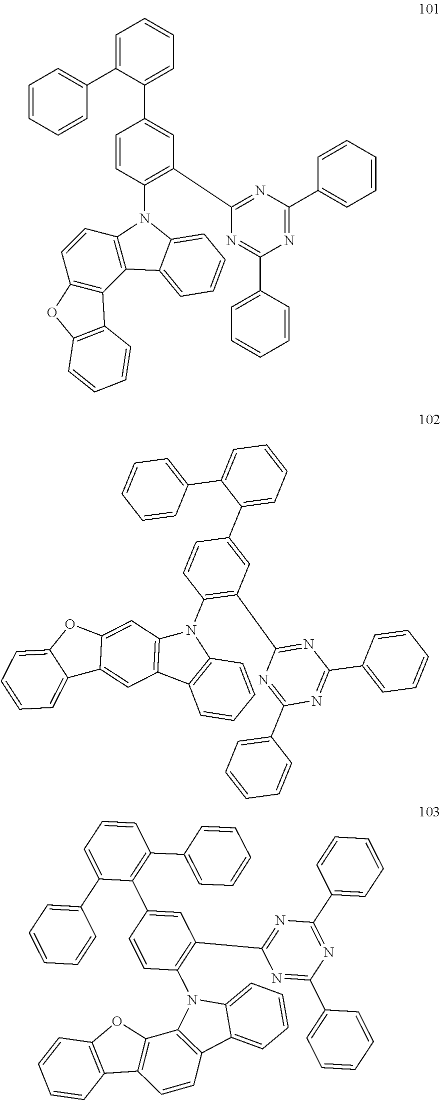

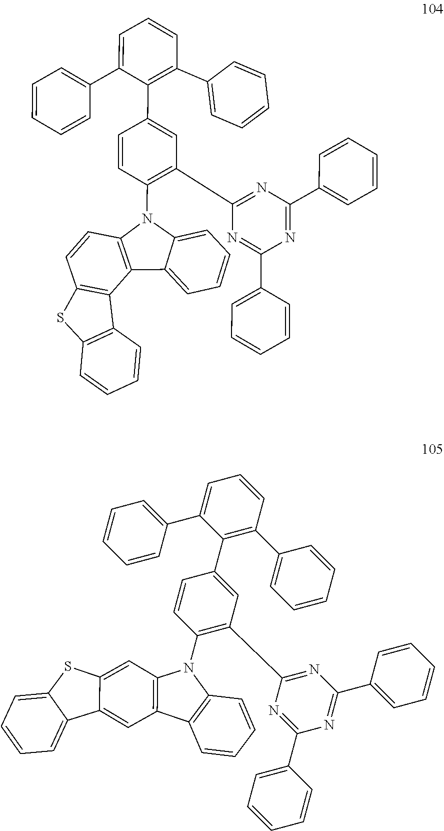 [SMILES: c1ccc(-c2nc(-c3ccccc3)nc(-c3cc(-c4c(-c5ccccc5)cccc4-c4ccccc4)ccc3-n3c4ccccc4c4c5c(ccc43)sc3ccccc35)n2)cc1 ; c1ccc(-c2nc(-c3ccccc3)nc(-c3cc(-c4c(-c5ccccc5)cccc4-c4ccccc4)ccc3-n3c4ccccc4c4cc5c(cc43)sc3ccccc35)n2)cc1]

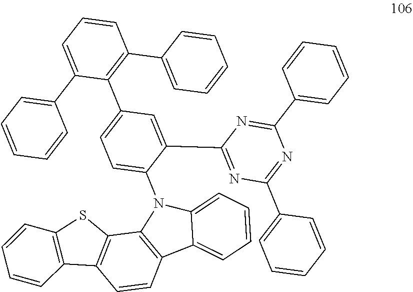 [SMILES: c1ccc(-c2nc(-c3ccccc3)nc(-c3cc(-c4c(-c5ccccc5)cccc4-c4ccccc4)ccc3-n3c4ccccc4c4ccc5c6ccccc6sc5c43)n2)cc1]

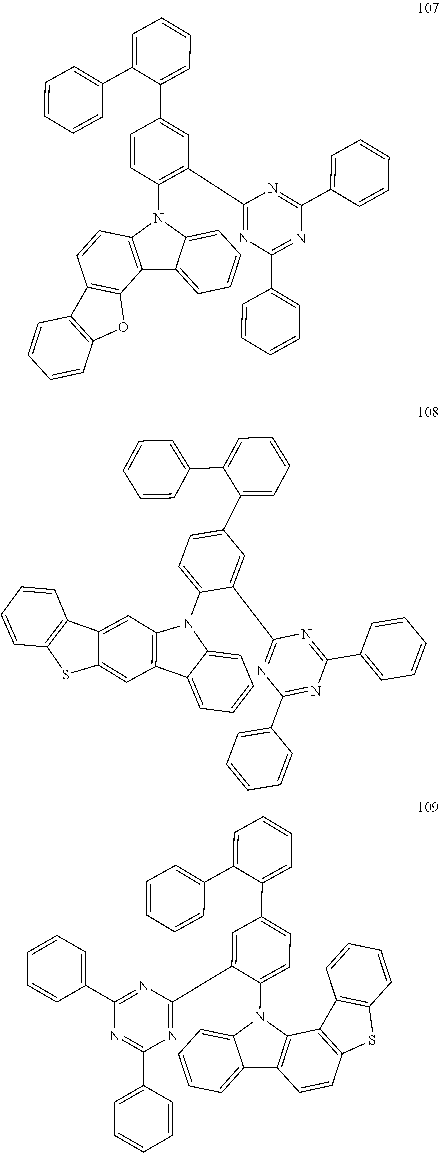

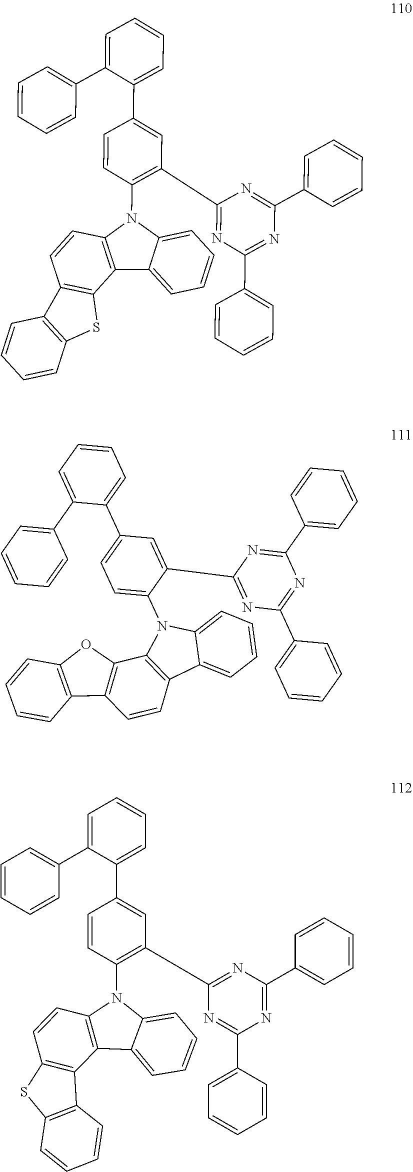 [SMILES: c1ccc(-c2nc(-c3ccccc3)nc(-c3cc(-c4ccccc4-c4ccccc4)ccc3-n3c4ccccc4c4c5sc6ccccc6c5ccc43)n2)cc1 ; c1ccc(-c2nc(-c3ccccc3)nc(-c3cc(-c4ccccc4-c4ccccc4)ccc3-n3c4ccccc4c4ccc5c6ccccc6oc5c43)n2)cc1 ; c1ccc(-c2nc(-c3ccccc3)nc(-c3cc(-c4ccccc4-c4ccccc4)ccc3-n3c4ccccc4c4c5c(ccc43)sc3ccccc35)n2)cc1]

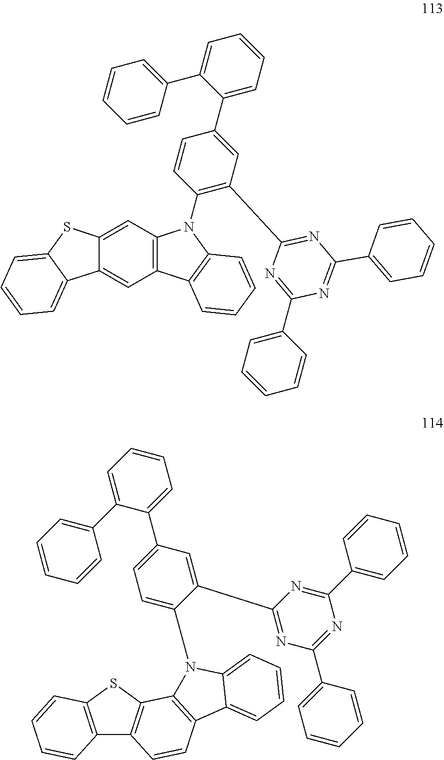 [SMILES: c1ccc(-c2nc(-c3ccccc3)nc(-c3cc(-c4ccccc4-c4ccccc4)ccc3-n3c4ccccc4c4cc5c(cc43)sc3ccccc35)n2)cc1 ; c1ccc(-c2nc(-c3ccccc3)nc(-c3cc(-c4ccccc4-c4ccccc4)ccc3-n3c4ccccc4c4ccc5c6ccccc6sc5c43)n2)cc1]

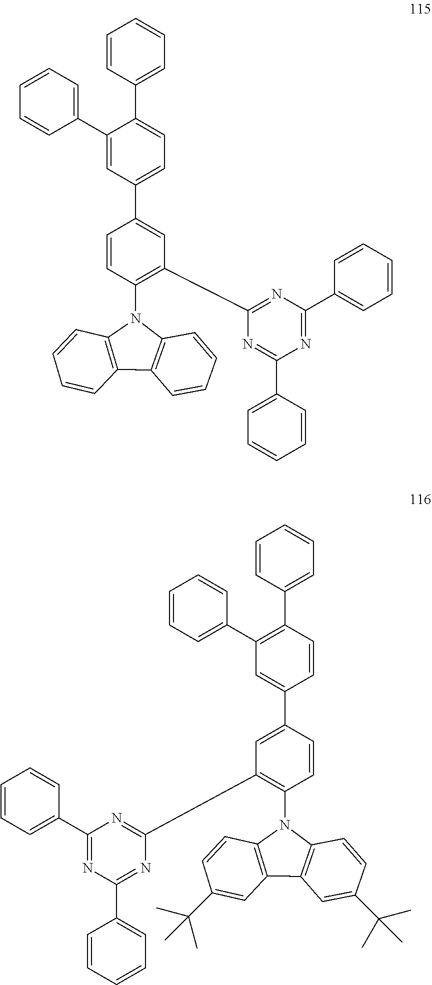 [SMILES: c1ccc(-c2nc(-c3ccccc3)nc(-c3cc(-c4ccc(-c5ccccc5)c(-c5ccccc5)c4)ccc3-n3c4ccccc4c4ccccc43)n2)cc1 ; CC(C)(C)c1ccc2c(c1)c1cc(C(C)(C)C)ccc1n2-c1ccc(-c2ccc(-c3ccccc3)c(-c3ccccc3)c2)cc1-c1nc(-c2ccccc2)nc(-c2ccccc2)n1]

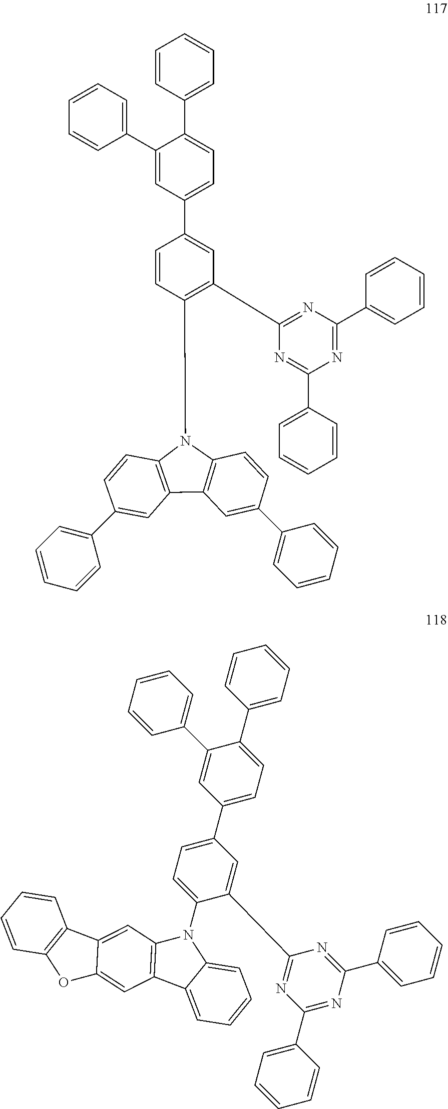 [SMILES: c1ccc(-c2ccc3c(c2)c2cc(-c4ccccc4)ccc2n3-c2ccc(-c3ccc(-c4ccccc4)c(-c4ccccc4)c3)cc2-c2nc(-c3ccccc3)nc(-c3ccccc3)n2)cc1 ; c1ccc(-c2nc(-c3ccccc3)nc(-c3cc(-c4ccc(-c5ccccc5)c(-c5ccccc5)c4)ccc3-n3c4ccccc4c4cc5oc6ccccc6c5cc43)n2)cc1]

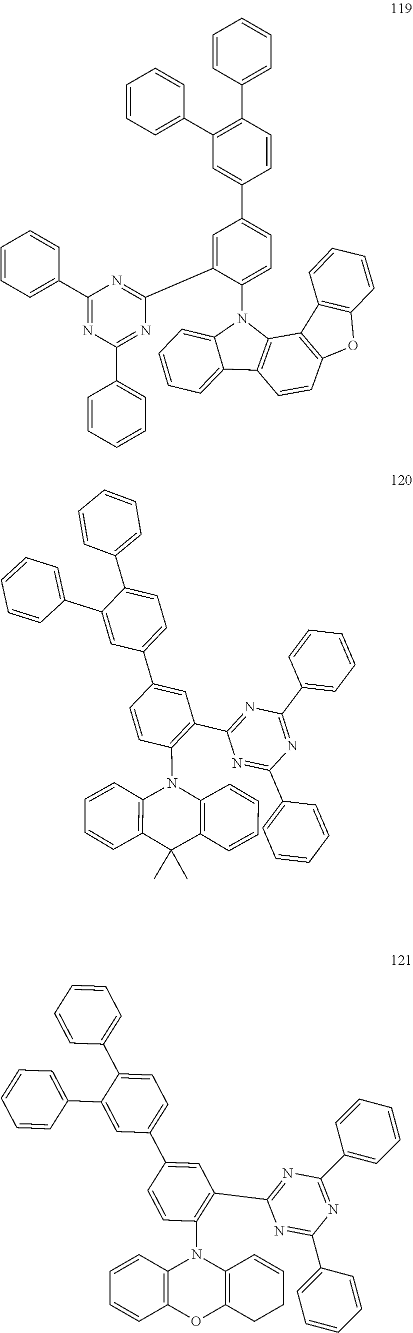

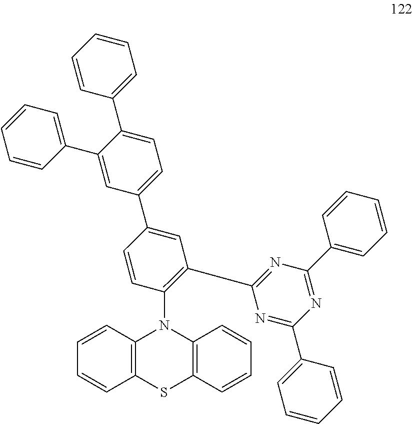 [SMILES: c1ccc(-c2nc(-c3ccccc3)nc(-c3cc(-c4ccc(-c5ccccc5)c(-c5ccccc5)c4)ccc3N3c4ccccc4Sc4ccccc43)n2)cc1]

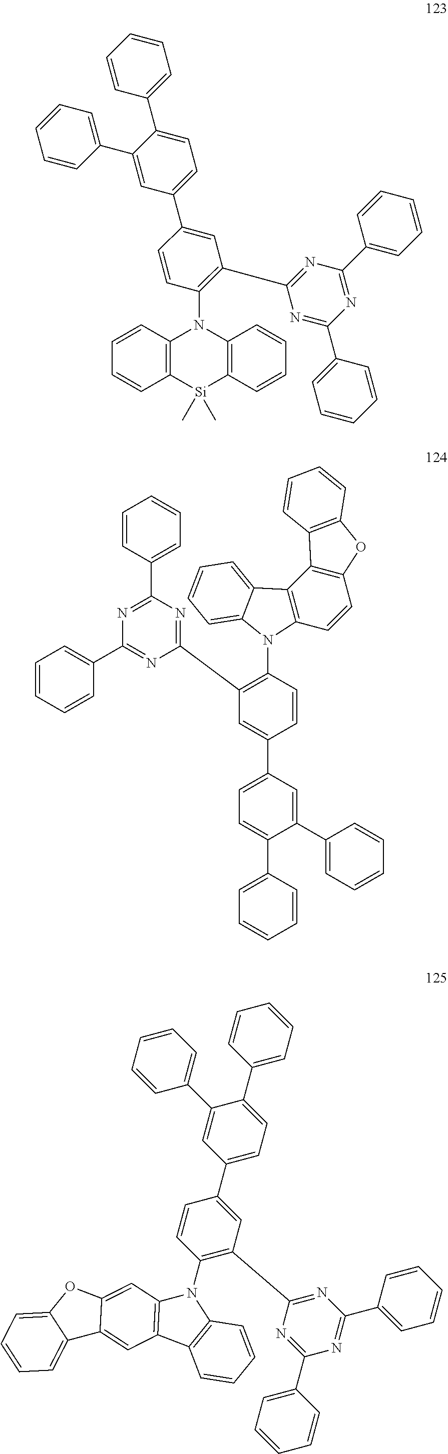 [SMILES: C[Si]1(C)c2ccccc2N(c2ccc(-c3ccc(-c4ccccc4)c(-c4ccccc4)c3)cc2-c2nc(-c3ccccc3)nc(-c3ccccc3)n2)c2ccccc21 ; c1ccc(-c2nc(-c3ccccc3)nc(-c3cc(-c4ccc(-c5ccccc5)c(-c5ccccc5)c4)ccc3-n3c4ccccc4c4c5c(ccc43)oc3ccccc35)n2)cc1 ; c1ccc(-c2nc(-c3ccccc3)nc(-c3cc(-c4ccc(-c5ccccc5)c(-c5ccccc5)c4)ccc3-n3c4ccccc4c4cc5c(cc43)oc3ccccc35)n2)cc1]

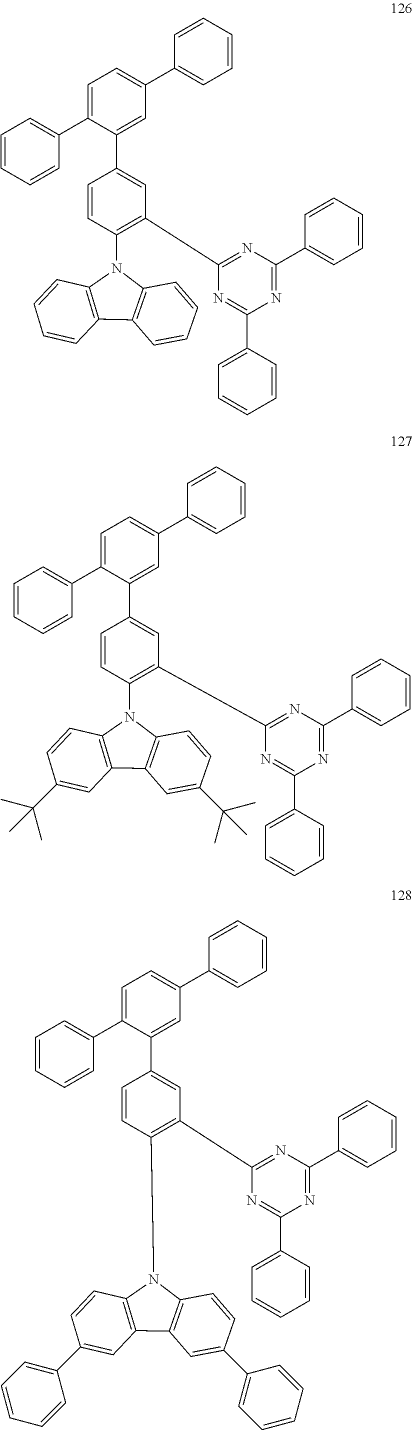 [SMILES: c1ccc(-c2ccc(-c3ccccc3)c(-c3ccc(-n4c5ccccc5c5ccccc54)c(-c4nc(-c5ccccc5)nc(-c5ccccc5)n4)c3)c2)cc1 ; CC(C)(C)c1ccc2c(c1)c1cc(C(C)(C)C)ccc1n2-c1ccc(-c2cc(-c3ccccc3)ccc2-c2ccccc2)cc1-c1nc(-c2ccccc2)nc(-c2ccccc2)n1 ; c1ccc(-c2ccc(-c3ccccc3)c(-c3ccc(-n4c5ccc(-c6ccccc6)cc5c5cc(-c6ccccc6)ccc54)c(-c4nc(-c5ccccc5)nc(-c5ccccc5)n4)c3)c2)cc1]

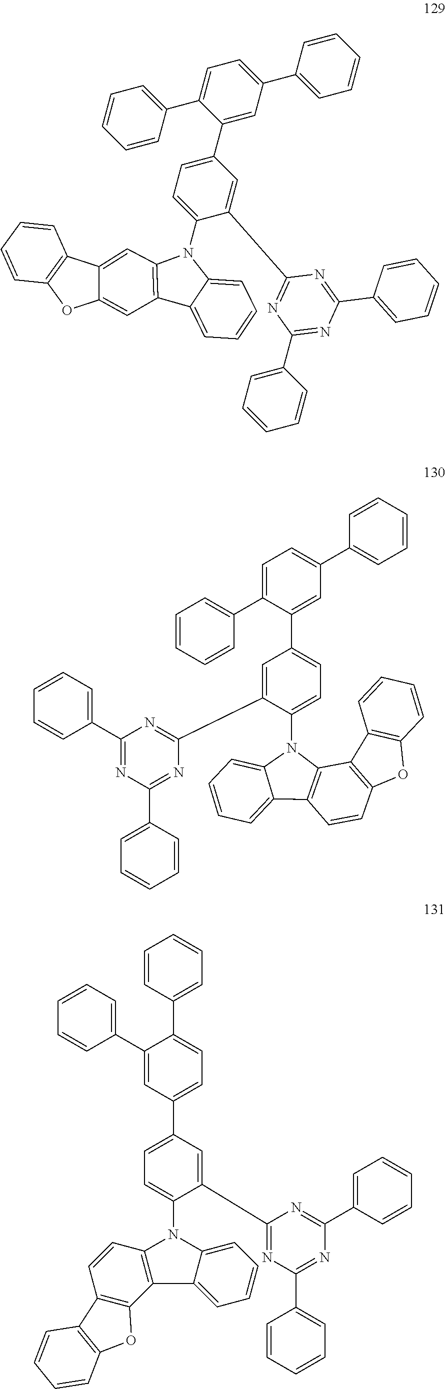 [SMILES: c1ccc(-c2ccc(-c3ccccc3)c(-c3ccc(-n4c5ccccc5c5cc6oc7ccccc7c6cc54)c(-c4nc(-c5ccccc5)nc(-c5ccccc5)n4)c3)c2)cc1 ; c1ccc(-c2ccc(-c3ccccc3)c(-c3ccc(-n4c5ccccc5c5ccc6oc7ccccc7c6c54)c(-c4nc(-c5ccccc5)nc(-c5ccccc5)n4)c3)c2)cc1 ; c1ccc(-c2nc(-c3ccccc3)nc(-c3cc(-c4ccc(-c5ccccc5)c(-c5ccccc5)c4)ccc3-n3c4ccccc4c4c5oc6ccccc6c5ccc43)n2)cc1]

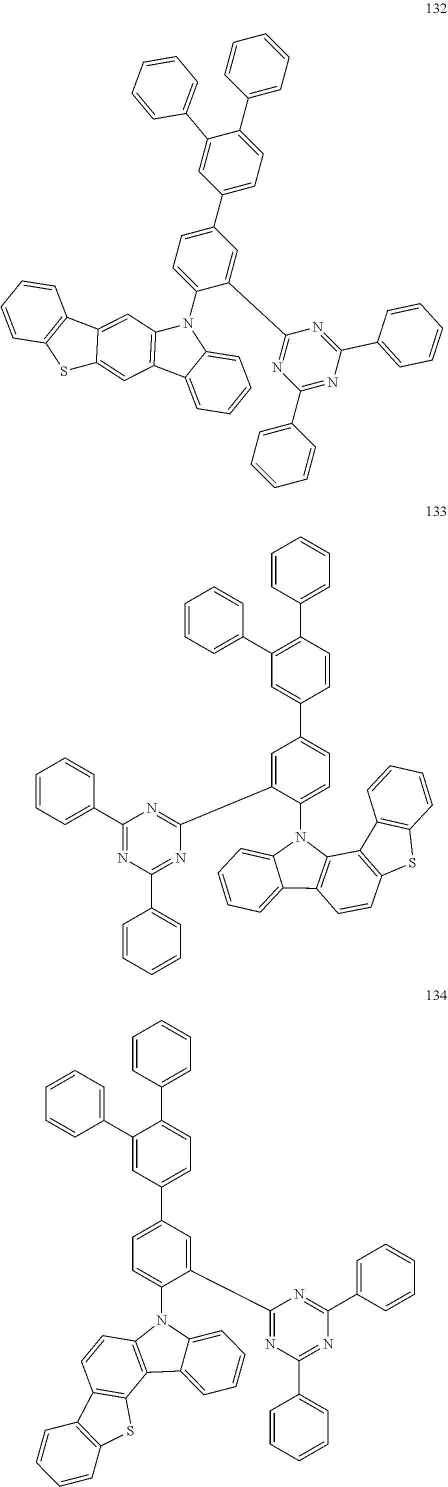 [SMILES: c1ccc(-c2nc(-c3ccccc3)nc(-c3cc(-c4ccc(-c5ccccc5)c(-c5ccccc5)c4)ccc3-n3c4ccccc4c4cc5sc6ccccc6c5cc43)n2)cc1 ; c1ccc(-c2nc(-c3ccccc3)nc(-c3cc(-c4ccc(-c5ccccc5)c(-c5ccccc5)c4)ccc3-n3c4ccccc4c4ccc5sc6ccccc6c5c43)n2)cc1 ; c1ccc(-c2nc(-c3ccccc3)nc(-c3cc(-c4ccc(-c5ccccc5)c(-c5ccccc5)c4)ccc3-n3c4ccccc4c4c5sc6ccccc6c5ccc43)n2)cc1]

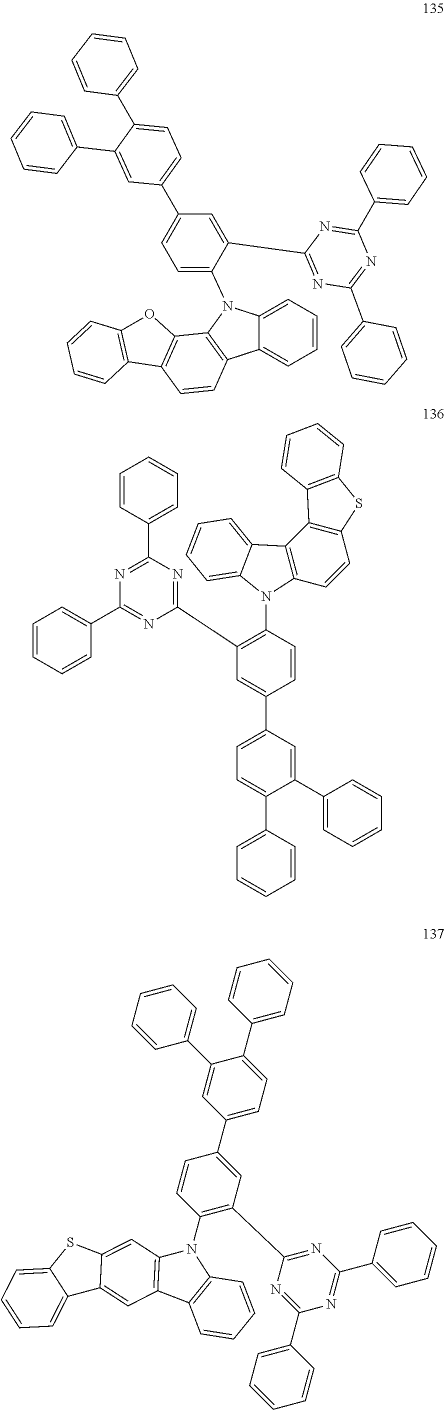 [SMILES: c1ccc(-c2nc(-c3ccccc3)nc(-c3cc(-c4ccc(-c5ccccc5)c(-c5ccccc5)c4)ccc3-n3c4ccccc4c4ccc5c6ccccc6oc5c43)n2)cc1 ; c1ccc(-c2nc(-c3ccccc3)nc(-c3cc(-c4ccc(-c5ccccc5)c(-c5ccccc5)c4)ccc3-n3c4ccccc4c4c5c(ccc43)sc3ccccc35)n2)cc1 ; c1ccc(-c2nc(-c3ccccc3)nc(-c3cc(-c4ccc(-c5ccccc5)c(-c5ccccc5)c4)ccc3-n3c4ccccc4c4cc5c(cc43)sc3ccccc35)n2)cc1]

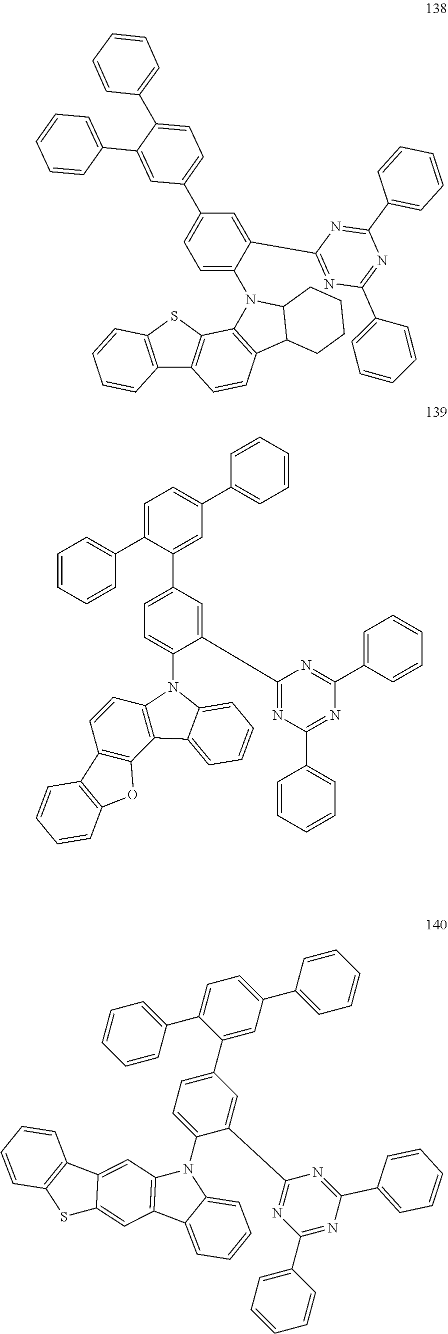 [SMILES: c1ccc(-c2nc(-c3ccccc3)nc(-c3cc(-c4ccc(-c5ccccc5)c(-c5ccccc5)c4)ccc3N3c4c(ccc5c4sc4ccccc45)C4CCCCC43)n2)cc1 ; c1ccc(-c2ccc(-c3ccccc3)c(-c3ccc(-n4c5ccccc5c5c6oc7ccccc7c6ccc54)c(-c4nc(-c5ccccc5)nc(-c5ccccc5)n4)c3)c2)cc1 ; c1ccc(-c2ccc(-c3ccccc3)c(-c3ccc(-n4c5ccccc5c5cc6sc7ccccc7c6cc54)c(-c4nc(-c5ccccc5)nc(-c5ccccc5)n4)c3)c2)cc1]

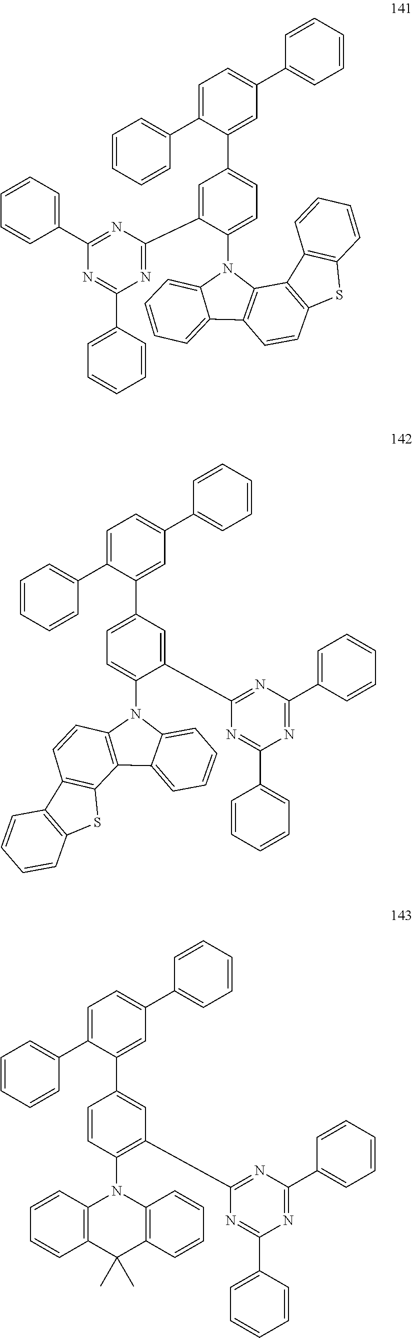 [SMILES: c1ccc(-c2ccc(-c3ccccc3)c(-c3ccc(-n4c5ccccc5c5ccc6sc7ccccc7c6c54)c(-c4nc(-c5ccccc5)nc(-c5ccccc5)n4)c3)c2)cc1 ; c1ccc(-c2ccc(-c3ccccc3)c(-c3ccc(-n4c5ccccc5c5c6sc7ccccc7c6ccc54)c(-c4nc(-c5ccccc5)nc(-c5ccccc5)n4)c3)c2)cc1 ; CC1(C)c2ccccc2N(c2ccc(-c3cc(-c4ccccc4)ccc3-c3ccccc3)cc2-c2nc(-c3ccccc3)nc(-c3ccccc3)n2)c2ccccc21]

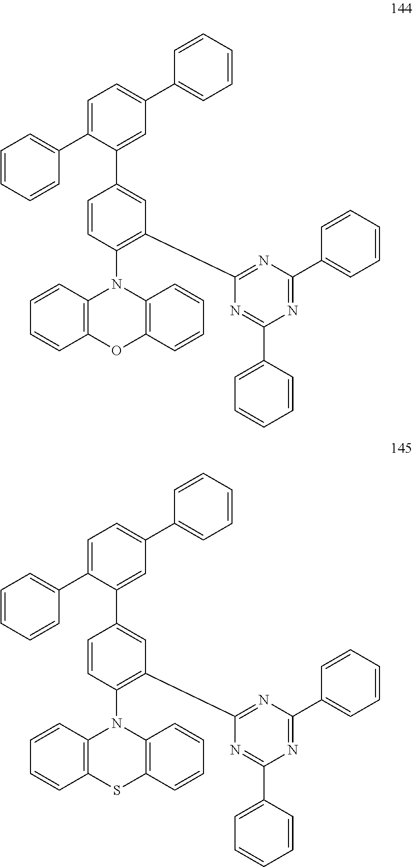 [SMILES: c1ccc(-c2ccc(-c3ccccc3)c(-c3ccc(N4c5ccccc5Oc5ccccc54)c(-c4nc(-c5ccccc5)nc(-c5ccccc5)n4)c3)c2)cc1 ; c1ccc(-c2ccc(-c3ccccc3)c(-c3ccc(N4c5ccccc5Sc5ccccc54)c(-c4nc(-c5ccccc5)nc(-c5ccccc5)n4)c3)c2)cc1]

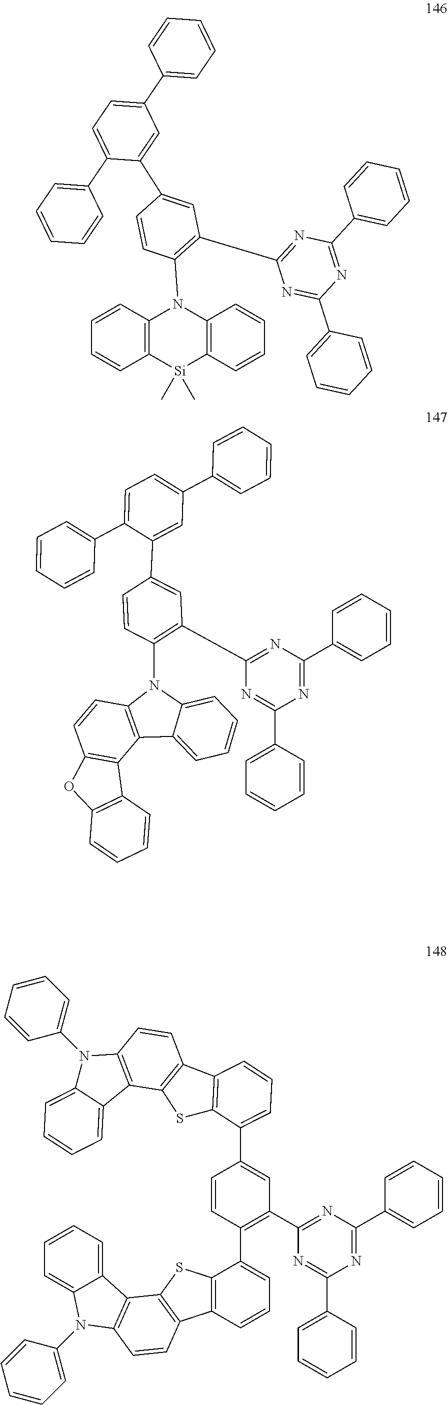 [SMILES: C[Si]1(C)c2ccccc2N(c2ccc(-c3cc(-c4ccccc4)ccc3-c3ccccc3)cc2-c2nc(-c3ccccc3)nc(-c3ccccc3)n2)c2ccccc21 ; c1ccc(-c2ccc(-c3ccccc3)c(-c3ccc(-n4c5ccccc5c5c6c(ccc54)oc4ccccc46)c(-c4nc(-c5ccccc5)nc(-c5ccccc5)n4)c3)c2)cc1 ; c1ccc(-c2nc(-c3ccccc3)nc(-c3cc(-c4cccc5c4sc4c5ccc5c4c4ccccc4n5-c4ccccc4)ccc3-c3cccc4c3sc3c4ccc4c3c3ccccc3n4-c3ccccc3)n2)cc1]

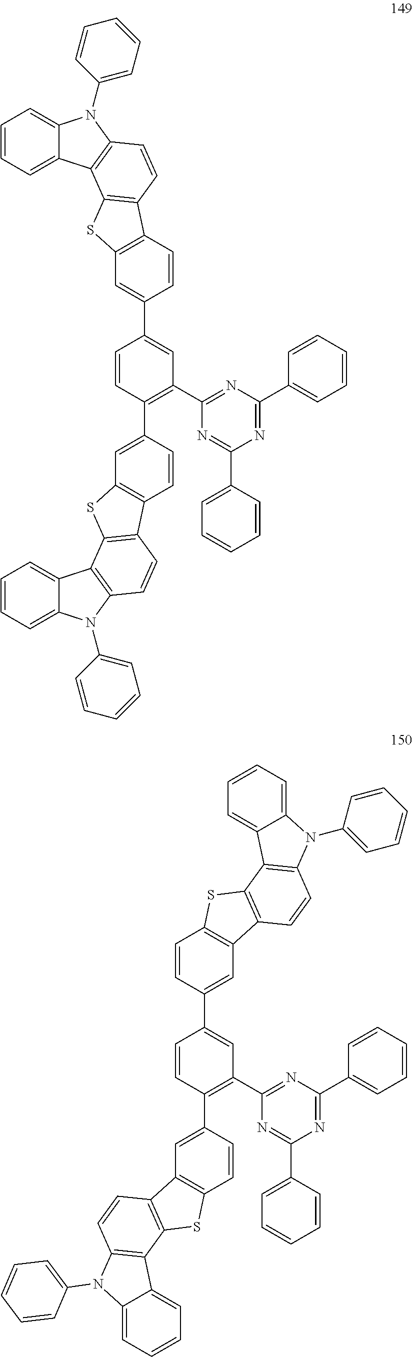 [SMILES: c1ccc(-c2nc(-c3ccccc3)nc(-c3cc(-c4ccc5c(c4)sc4c5ccc5c4c4ccccc4n5-c4ccccc4)ccc3-c3ccc4c(c3)sc3c4ccc4c3c3ccccc3n4-c3ccccc3)n2)cc1 ; c1ccc(-c2nc(-c3ccccc3)nc(-c3cc(-c4ccc5sc6c(ccc7c6c6ccccc6n7-c6ccccc6)c5c4)ccc3-c3ccc4sc5c(ccc6c5c5ccccc5n6-c5ccccc5)c4c3)n2)cc1]

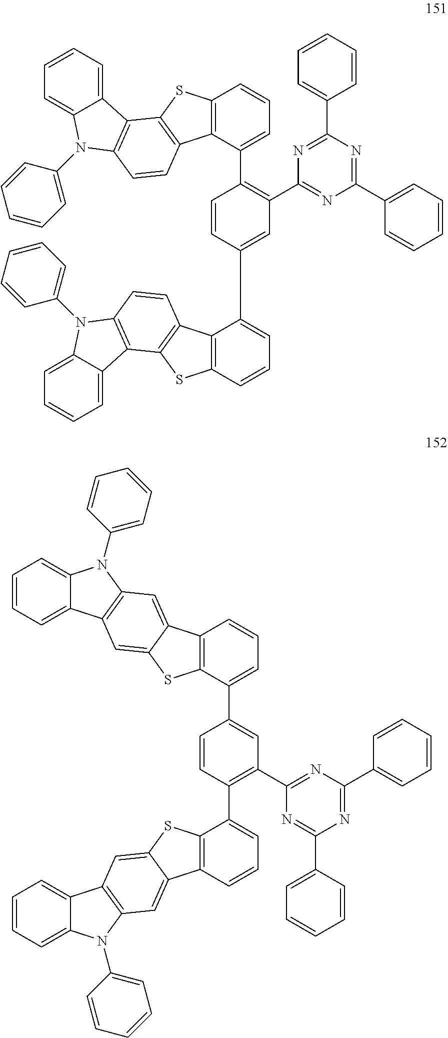 [SMILES: c1ccc(-c2nc(-c3ccccc3)nc(-c3cc(-c4cccc5sc6c(ccc7c6c6ccccc6n7-c6ccccc6)c45)ccc3-c3cccc4sc5c(ccc6c5c5ccccc5n6-c5ccccc5)c34)n2)cc1 ; c1ccc(-c2nc(-c3ccccc3)nc(-c3cc(-c4cccc5c4sc4cc6c7ccccc7n(-c7ccccc7)c6cc45)ccc3-c3cccc4c3sc3cc5c6ccccc6n(-c6ccccc6)c5cc34)n2)cc1]

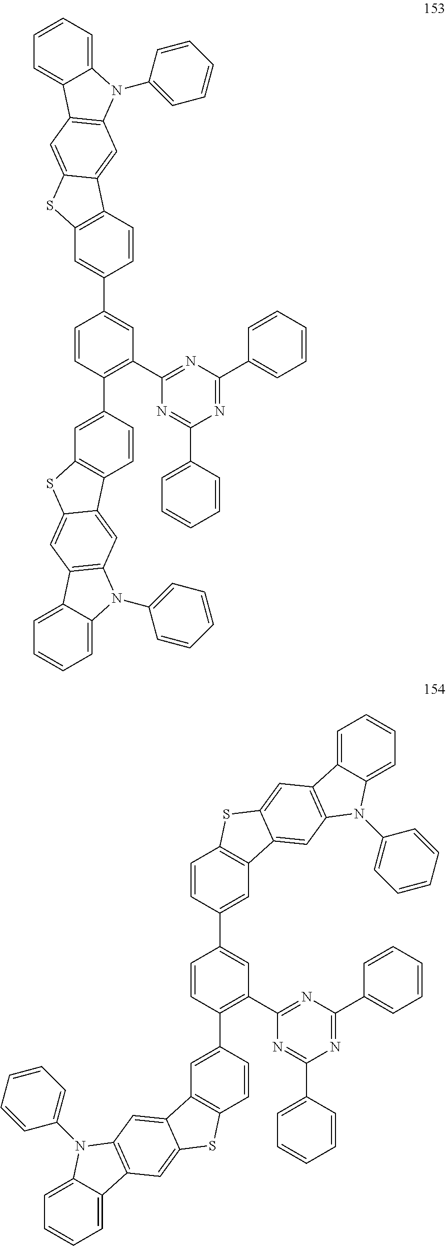 [SMILES: c1ccc(-c2nc(-c3ccccc3)nc(-c3cc(-c4ccc5c(c4)sc4cc6c7ccccc7n(-c7ccccc7)c6cc45)ccc3-c3ccc4c(c3)sc3cc5c6ccccc6n(-c6ccccc6)c5cc34)n2)cc1 ; c1ccc(-c2nc(-c3ccccc3)nc(-c3cc(-c4ccc5sc6cc7c8ccccc8n(-c8ccccc8)c7cc6c5c4)ccc3-c3ccc4sc5cc6c7ccccc7n(-c7ccccc7)c6cc5c4c3)n2)cc1]

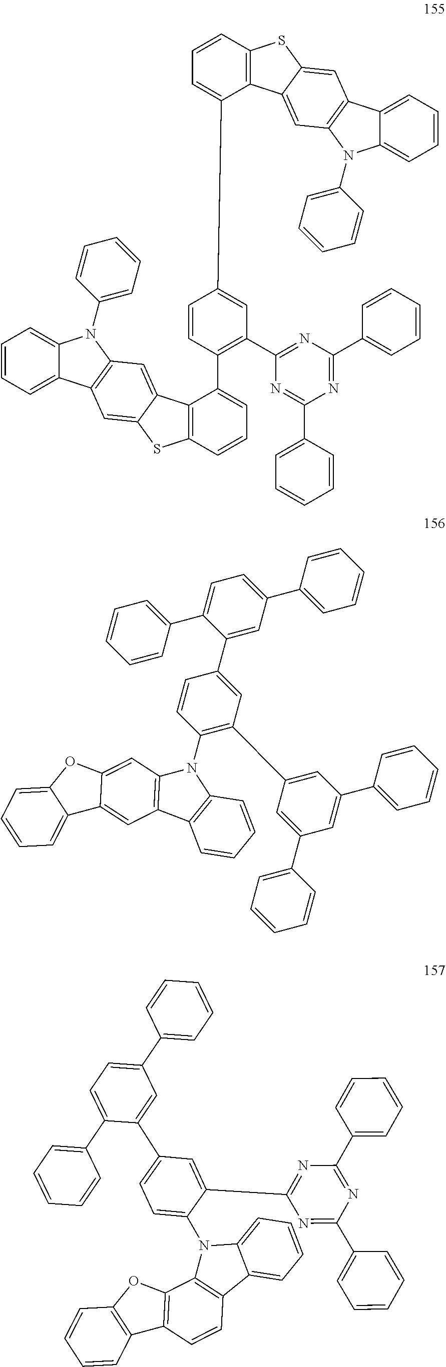 [SMILES: c1ccc(-c2nc(-c3ccccc3)nc(-c3cc(-c4cccc5sc6cc7c8ccccc8n(-c8ccccc8)c7cc6c45)ccc3-c3cccc4sc5cc6c7ccccc7n(-c7ccccc7)c6cc5c34)n2)cc1 ; c1ccc(-c2cc(-c3ccccc3)cc(-c3cc(-c4cc(-c5ccccc5)ccc4-c4ccccc4)ccc3-n3c4ccccc4c4cc5c(cc43)oc3ccccc35)c2)cc1 ; c1ccc(-c2ccc(-c3ccccc3)c(-c3ccc(-n4c5ccccc5c5ccc6c7ccccc7oc6c54)c(-c4nc(-c5ccccc5)nc(-c5ccccc5)n4)c3)c2)cc1]

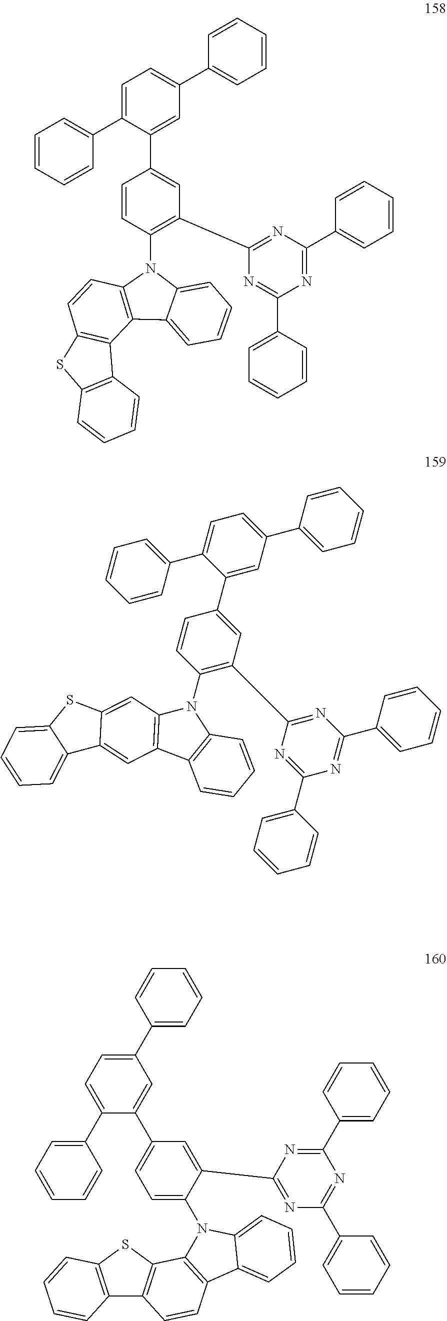 [SMILES: c1ccc(-c2ccc(-c3ccccc3)c(-c3ccc(-n4c5ccccc5c5c6c(ccc54)sc4ccccc46)c(-c4nc(-c5ccccc5)nc(-c5ccccc5)n4)c3)c2)cc1 ; c1ccc(-c2ccc(-c3ccccc3)c(-c3ccc(-n4c5ccccc5c5cc6c(cc54)sc4ccccc46)c(-c4nc(-c5ccccc5)nc(-c5ccccc5)n4)c3)c2)cc1 ; c1ccc(-c2ccc(-c3ccccc3)c(-c3ccc(-n4c5ccccc5c5ccc6c7ccccc7sc6c54)c(-c4nc(-c5ccccc5)nc(-c5ccccc5)n4)c3)c2)cc1]

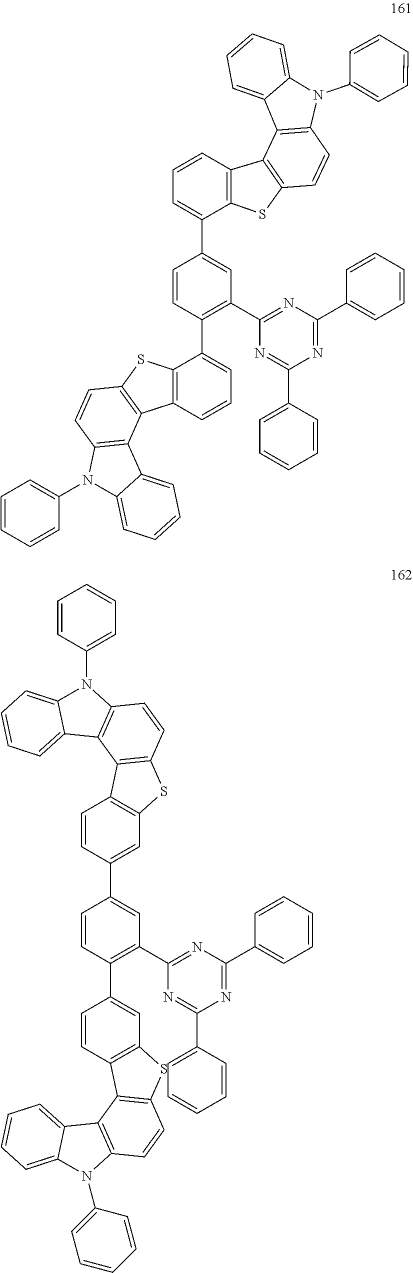 [SMILES: c1ccc(-c2nc(-c3ccccc3)nc(-c3cc(-c4cccc5c4sc4ccc6c(c7ccccc7n6-c6ccccc6)c45)ccc3-c3cccc4c3sc3ccc5c(c6ccccc6n5-c5ccccc5)c34)n2)cc1 ; c1ccc(-c2nc(-c3ccccc3)nc(-c3cc(-c4ccc5c(c4)sc4ccc6c(c7ccccc7n6-c6ccccc6)c45)ccc3-c3ccc4c(c3)sc3ccc5c(c6ccccc6n5-c5ccccc5)c34)n2)cc1]

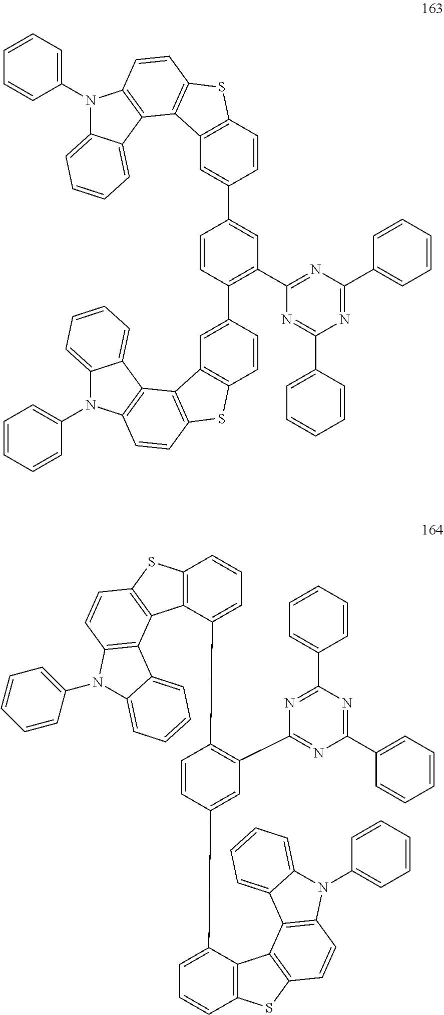 [SMILES: c1ccc(-c2nc(-c3ccccc3)nc(-c3cc(-c4ccc5sc6ccc7c(c8ccccc8n7-c7ccccc7)c6c5c4)ccc3-c3ccc4sc5ccc6c(c7ccccc7n6-c6ccccc6)c5c4c3)n2)cc1 ; c1ccc(-c2nc(-c3ccccc3)nc(-c3cc(-c4cccc5sc6ccc7c(c8ccccc8n7-c7ccccc7)c6c45)ccc3-c3cccc4sc5ccc6c(c7ccccc7n6-c6ccccc6)c5c34)n2)cc1]

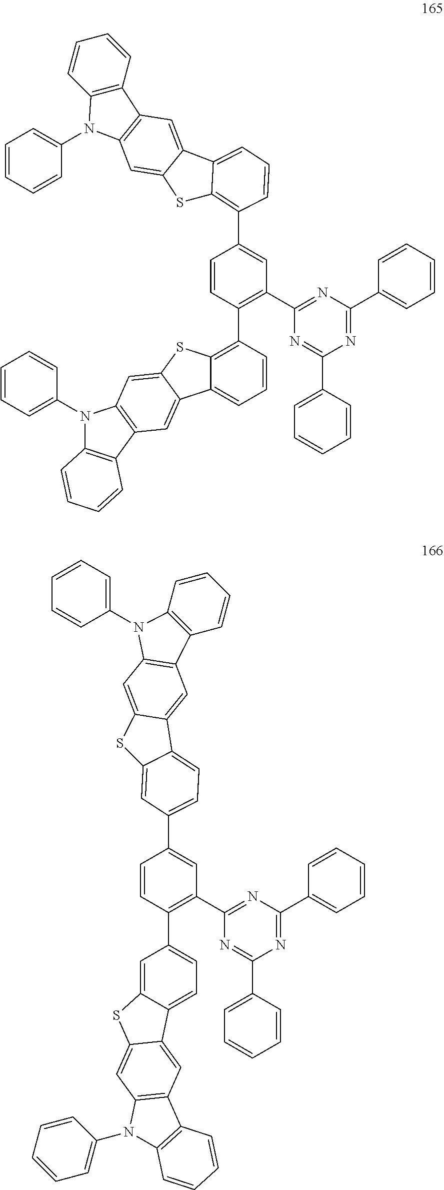 [SMILES: c1ccc(-c2nc(-c3ccccc3)nc(-c3cc(-c4cccc5c4sc4cc6c(cc45)c4ccccc4n6-c4ccccc4)ccc3-c3cccc4c3sc3cc5c(cc34)c3ccccc3n5-c3ccccc3)n2)cc1 ; c1ccc(-c2nc(-c3ccccc3)nc(-c3cc(-c4ccc5c(c4)sc4cc6c(cc45)c4ccccc4n6-c4ccccc4)ccc3-c3ccc4c(c3)sc3cc5c(cc34)c3ccccc3n5-c3ccccc3)n2)cc1]

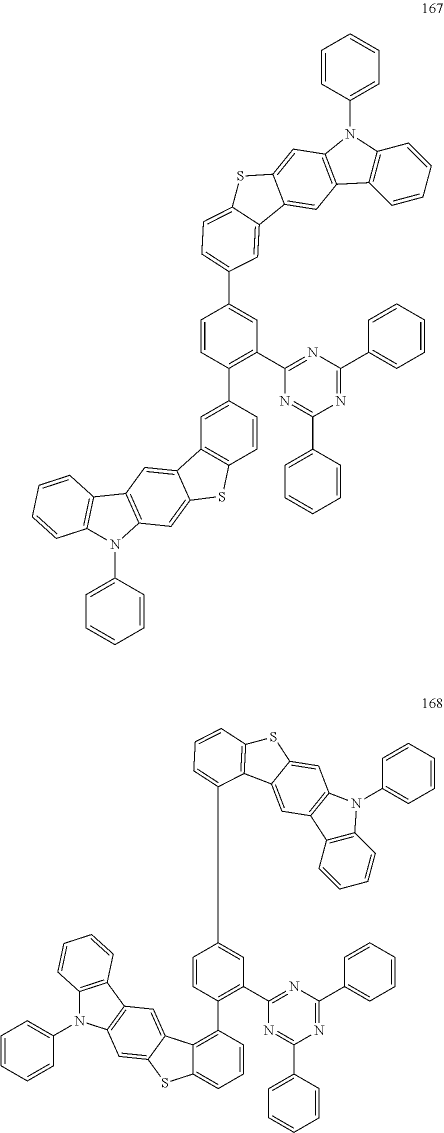 [SMILES: c1ccc(-c2nc(-c3ccccc3)nc(-c3cc(-c4ccc5sc6cc7c(cc6c5c4)c4ccccc4n7-c4ccccc4)ccc3-c3ccc4sc5cc6c(cc5c4c3)c3ccccc3n6-c3ccccc3)n2)cc1 ; c1ccc(-c2nc(-c3ccccc3)nc(-c3cc(-c4cccc5sc6cc7c(cc6c45)c4ccccc4n7-c4ccccc4)ccc3-c3cccc4sc5cc6c(cc5c34)c3ccccc3n6-c3ccccc3)n2)cc1]

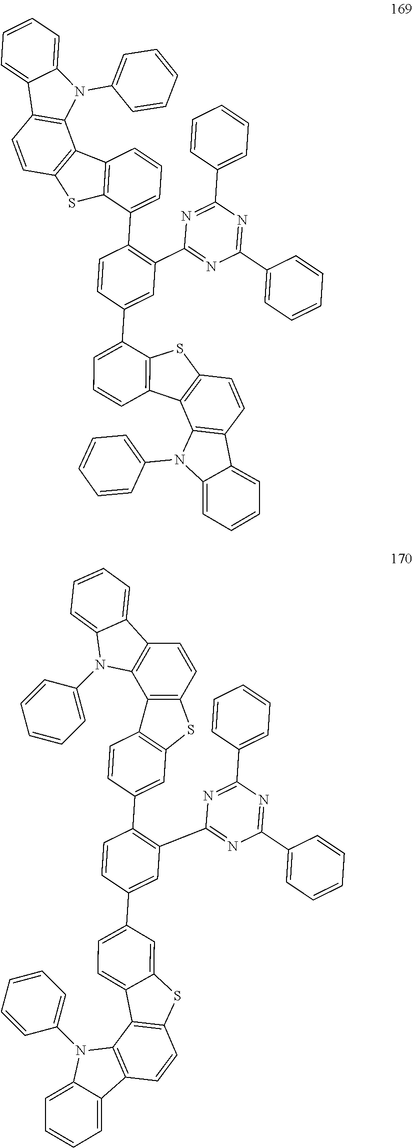 [SMILES: c1ccc(-c2nc(-c3ccccc3)nc(-c3cc(-c4cccc5c4sc4ccc6c7ccccc7n(-c7ccccc7)c6c45)ccc3-c3cccc4c3sc3ccc5c6ccccc6n(-c6ccccc6)c5c34)n2)cc1 ; c1ccc(-c2nc(-c3ccccc3)nc(-c3cc(-c4ccc5c(c4)sc4ccc6c7ccccc7n(-c7ccccc7)c6c45)ccc3-c3ccc4c(c3)sc3ccc5c6ccccc6n(-c6ccccc6)c5c34)n2)cc1]

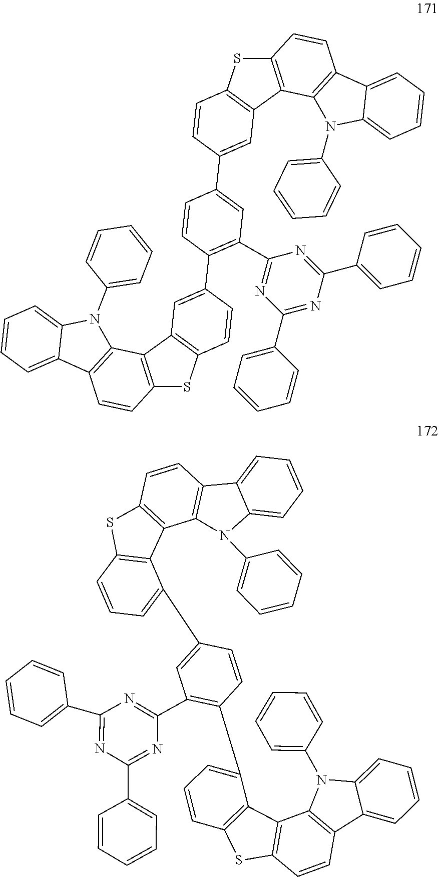

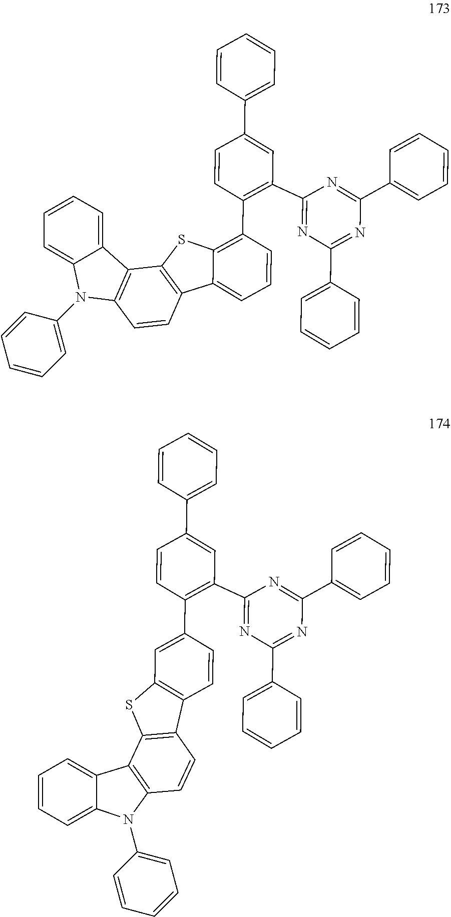 [SMILES: c1ccc(-c2ccc(-c3cccc4c3sc3c4ccc4c3c3ccccc3n4-c3ccccc3)c(-c3nc(-c4ccccc4)nc(-c4ccccc4)n3)c2)cc1 ; c1ccc(-c2ccc(-c3ccc4c(c3)sc3c4ccc4c3c3ccccc3n4-c3ccccc3)c(-c3nc(-c4ccccc4)nc(-c4ccccc4)n3)c2)cc1]

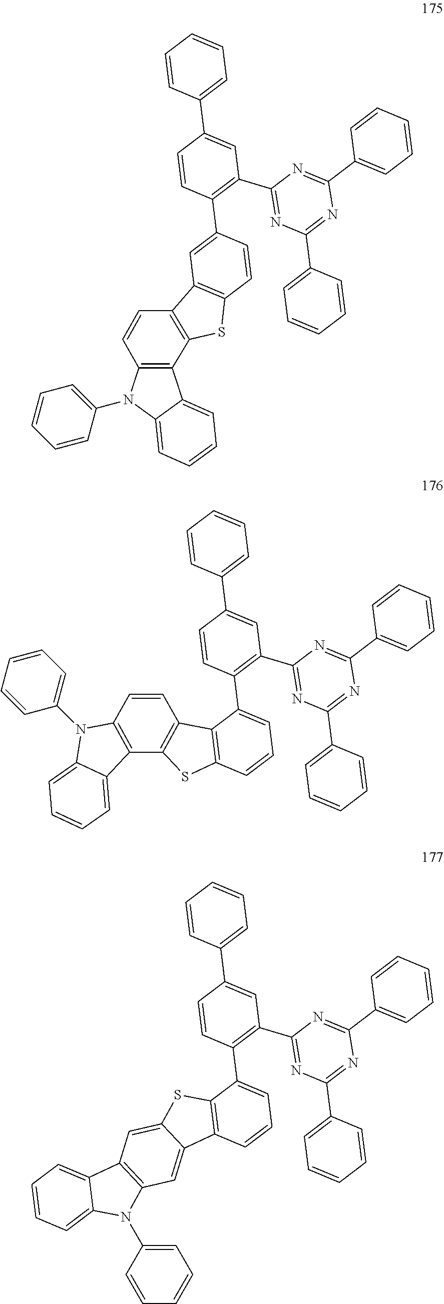 [SMILES: c1ccc(-c2ccc(-c3ccc4sc5c(ccc6c5c5ccccc5n6-c5ccccc5)c4c3)c(-c3nc(-c4ccccc4)nc(-c4ccccc4)n3)c2)cc1 ; c1ccc(-c2ccc(-c3cccc4sc5c(ccc6c5c5ccccc5n6-c5ccccc5)c34)c(-c3nc(-c4ccccc4)nc(-c4ccccc4)n3)c2)cc1 ; c1ccc(-c2ccc(-c3cccc4c3sc3cc5c6ccccc6n(-c6ccccc6)c5cc34)c(-c3nc(-c4ccccc4)nc(-c4ccccc4)n3)c2)cc1]

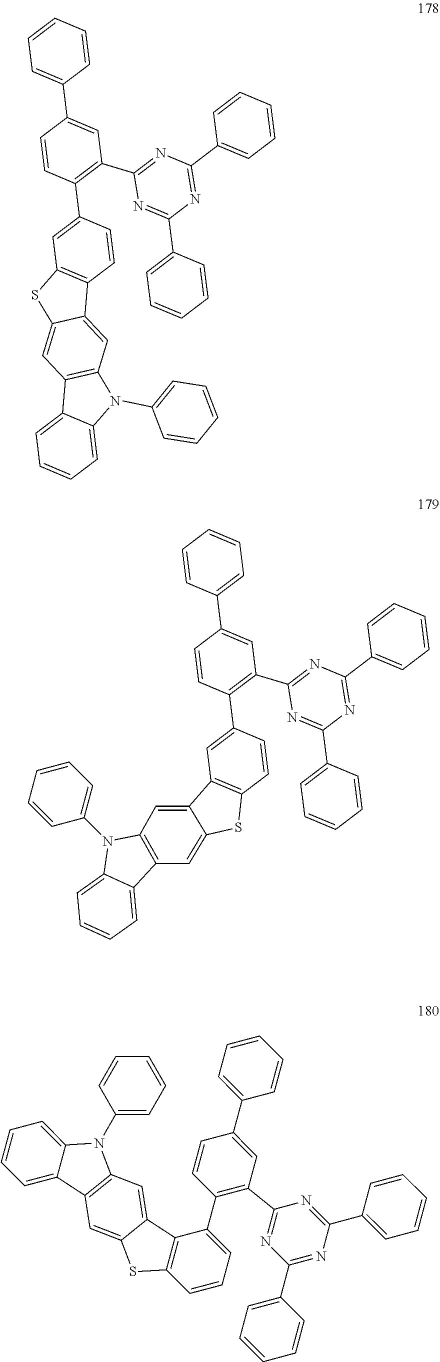 [SMILES: c1ccc(-c2ccc(-c3ccc4c(c3)sc3cc5c6ccccc6n(-c6ccccc6)c5cc34)c(-c3nc(-c4ccccc4)nc(-c4ccccc4)n3)c2)cc1 ; c1ccc(-c2ccc(-c3ccc4sc5cc6c7ccccc7n(-c7ccccc7)c6cc5c4c3)c(-c3nc(-c4ccccc4)nc(-c4ccccc4)n3)c2)cc1 ; c1ccc(-c2ccc(-c3cccc4sc5cc6c7ccccc7n(-c7ccccc7)c6cc5c34)c(-c3nc(-c4ccccc4)nc(-c4ccccc4)n3)c2)cc1]

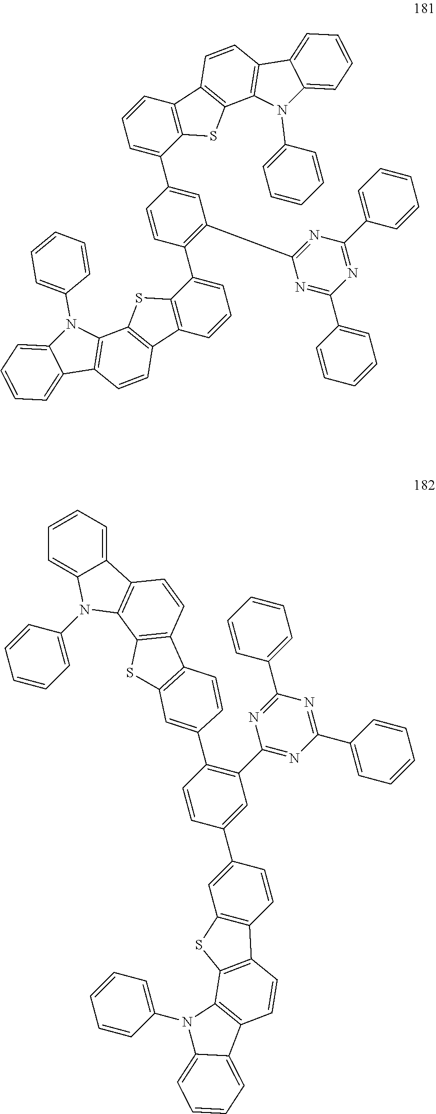 [SMILES: c1ccc(-c2nc(-c3ccccc3)nc(-c3cc(-c4cccc5c4sc4c5ccc5c6ccccc6n(-c6ccccc6)c54)ccc3-c3cccc4c3sc3c4ccc4c5ccccc5n(-c5ccccc5)c43)n2)cc1 ; c1ccc(-c2nc(-c3ccccc3)nc(-c3cc(-c4ccc5c(c4)sc4c5ccc5c6ccccc6n(-c6ccccc6)c54)ccc3-c3ccc4c(c3)sc3c4ccc4c5ccccc5n(-c5ccccc5)c43)n2)cc1]

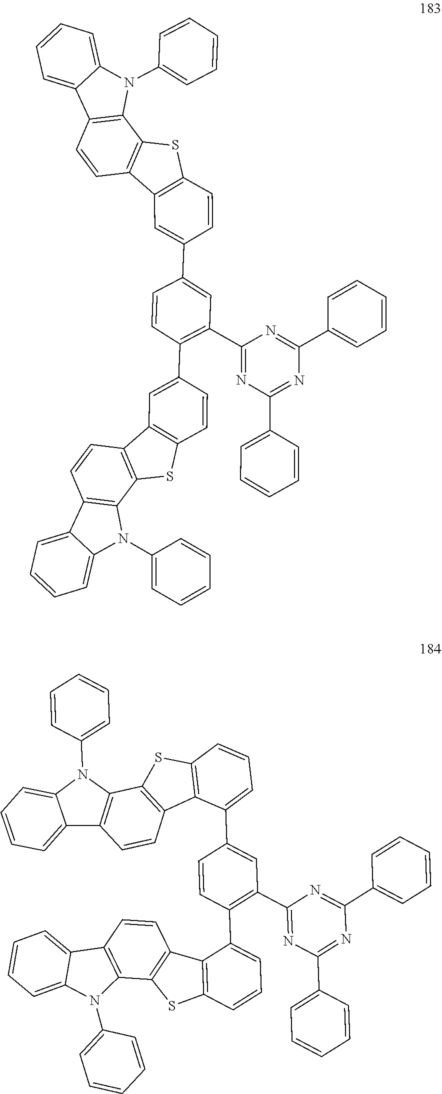 [SMILES: c1ccc(-c2nc(-c3ccccc3)nc(-c3cc(-c4ccc5sc6c(ccc7c8ccccc8n(-c8ccccc8)c76)c5c4)ccc3-c3ccc4sc5c(ccc6c7ccccc7n(-c7ccccc7)c65)c4c3)n2)cc1 ; c1ccc(-c2nc(-c3ccccc3)nc(-c3cc(-c4cccc5sc6c(ccc7c8ccccc8n(-c8ccccc8)c76)c45)ccc3-c3cccc4sc5c(ccc6c7ccccc7n(-c7ccccc7)c65)c34)n2)cc1]

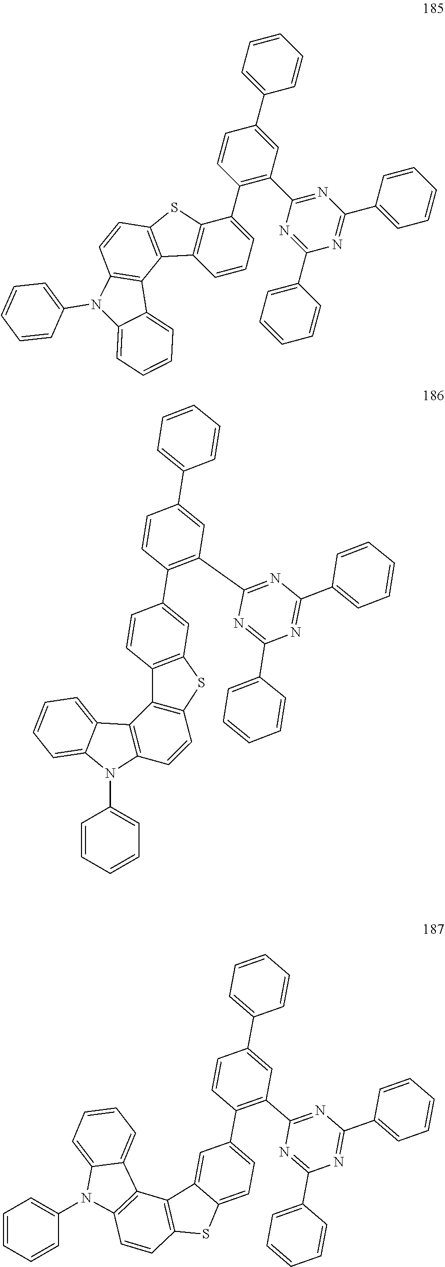 [SMILES: c1ccc(-c2ccc(-c3cccc4c3sc3ccc5c(c6ccccc6n5-c5ccccc5)c34)c(-c3nc(-c4ccccc4)nc(-c4ccccc4)n3)c2)cc1 ; c1ccc(-c2ccc(-c3ccc4c(c3)sc3ccc5c(c6ccccc6n5-c5ccccc5)c34)c(-c3nc(-c4ccccc4)nc(-c4ccccc4)n3)c2)cc1 ; c1ccc(-c2ccc(-c3ccc4sc5ccc6c(c7ccccc7n6-c6ccccc6)c5c4c3)c(-c3nc(-c4ccccc4)nc(-c4ccccc4)n3)c2)cc1]

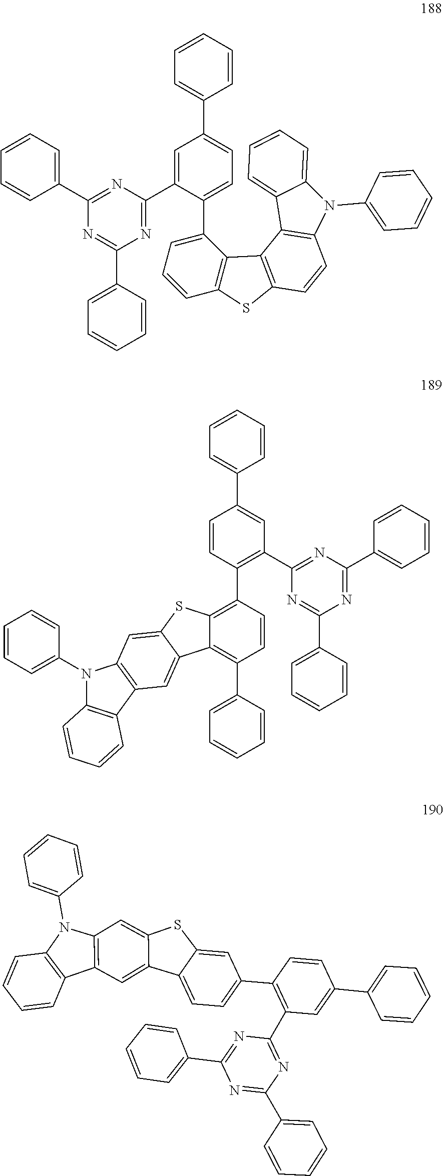 [SMILES: c1ccc(-c2ccc(-c3cccc4sc5ccc6c(c7ccccc7n6-c6ccccc6)c5c34)c(-c3nc(-c4ccccc4)nc(-c4ccccc4)n3)c2)cc1 ; c1ccc(-c2ccc(-c3ccc(-c4ccccc4)c4c3sc3cc5c(cc34)c3ccccc3n5-c3ccccc3)c(-c3nc(-c4ccccc4)nc(-c4ccccc4)n3)c2)cc1 ; c1ccc(-c2ccc(-c3ccc4c(c3)sc3cc5c(cc34)c3ccccc3n5-c3ccccc3)c(-c3nc(-c4ccccc4)nc(-c4ccccc4)n3)c2)cc1]

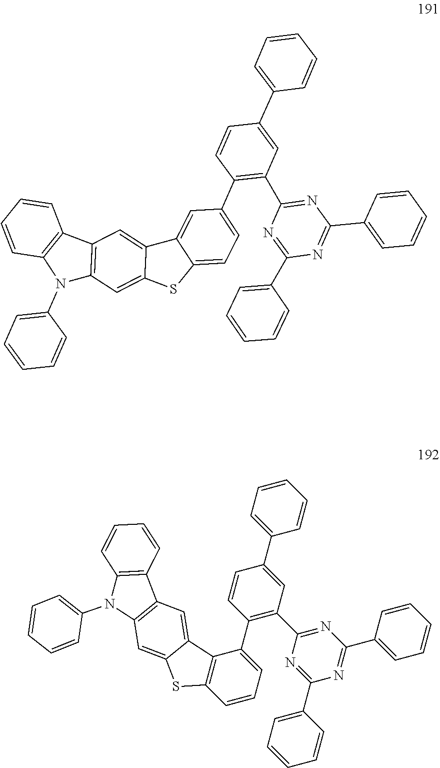 [SMILES: c1ccc(-c2ccc(-c3ccc4sc5cc6c(cc5c4c3)c3ccccc3n6-c3ccccc3)c(-c3nc(-c4ccccc4)nc(-c4ccccc4)n3)c2)cc1 ; c1ccc(-c2ccc(-c3cccc4sc5cc6c(cc5c34)c3ccccc3n6-c3ccccc3)c(-c3nc(-c4ccccc4)nc(-c4ccccc4)n3)c2)cc1]

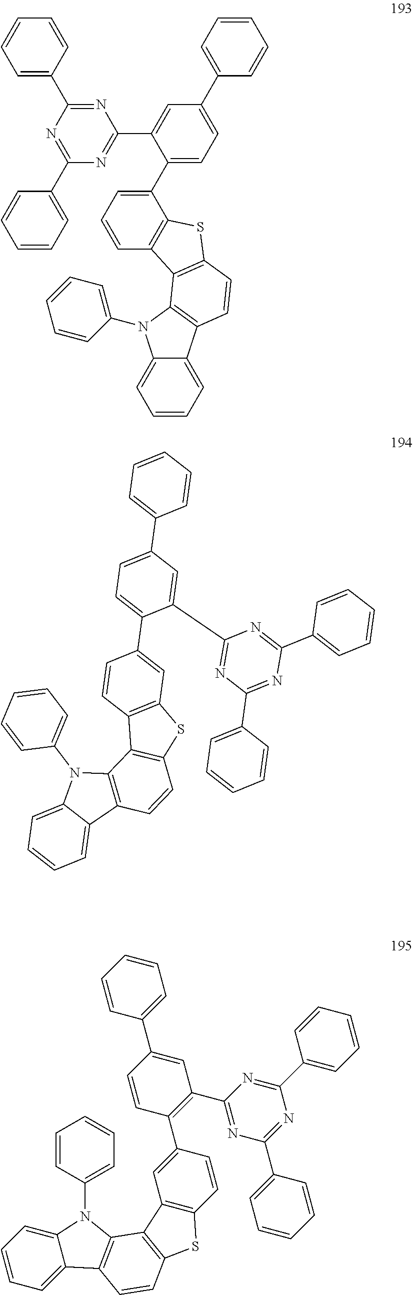

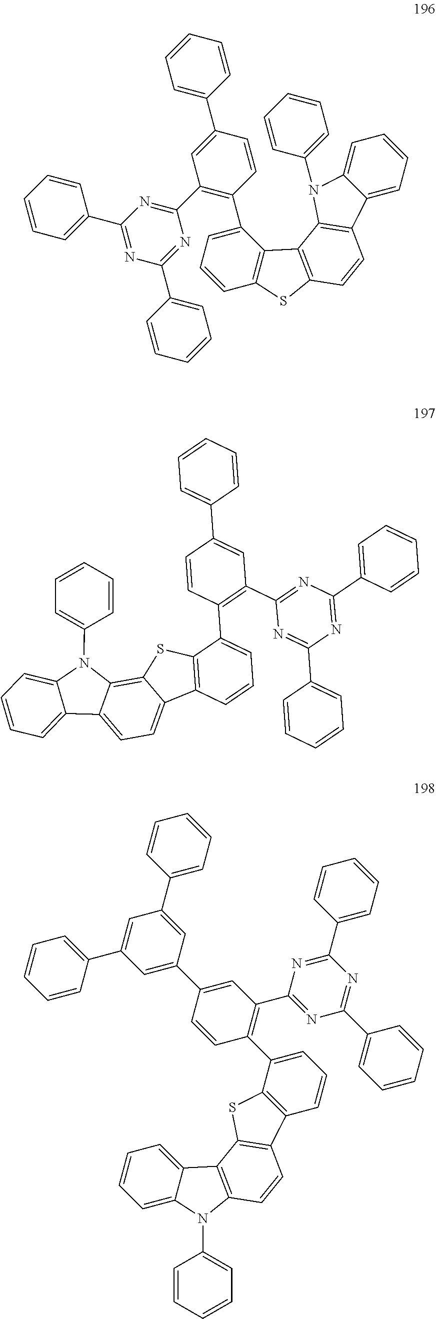 [SMILES: c1ccc(-c2ccc(-c3cccc4sc5ccc6c7ccccc7n(-c7ccccc7)c6c5c34)c(-c3nc(-c4ccccc4)nc(-c4ccccc4)n3)c2)cc1 ; c1ccc(-c2ccc(-c3cccc4c3sc3c4ccc4c5ccccc5n(-c5ccccc5)c43)c(-c3nc(-c4ccccc4)nc(-c4ccccc4)n3)c2)cc1 ; c1ccc(-c2cc(-c3ccccc3)cc(-c3ccc(-c4cccc5c4sc4c5ccc5c4c4ccccc4n5-c4ccccc4)c(-c4nc(-c5ccccc5)nc(-c5ccccc5)n4)c3)c2)cc1]

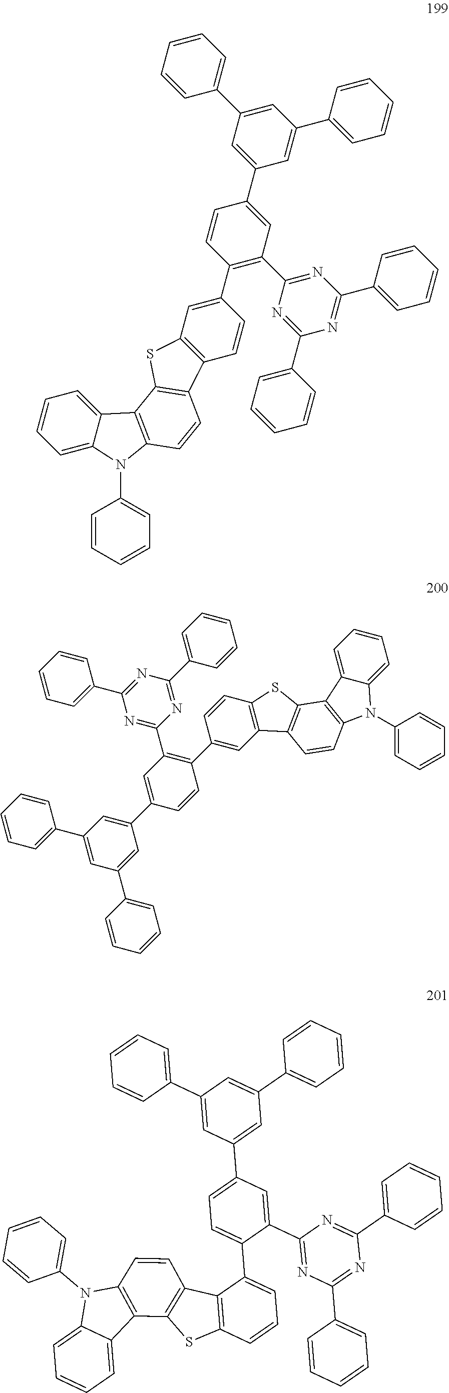 [SMILES: c1ccc(-c2cc(-c3ccccc3)cc(-c3ccc(-c4ccc5c(c4)sc4c5ccc5c4c4ccccc4n5-c4ccccc4)c(-c4nc(-c5ccccc5)nc(-c5ccccc5)n4)c3)c2)cc1 ; c1ccc(-c2cc(-c3ccccc3)cc(-c3ccc(-c4ccc5sc6c(ccc7c6c6ccccc6n7-c6ccccc6)c5c4)c(-c4nc(-c5ccccc5)nc(-c5ccccc5)n4)c3)c2)cc1 ; c1ccc(-c2cc(-c3ccccc3)cc(-c3ccc(-c4cccc5sc6c(ccc7c6c6ccccc6n7-c6ccccc6)c45)c(-c4nc(-c5ccccc5)nc(-c5ccccc5)n4)c3)c2)cc1]

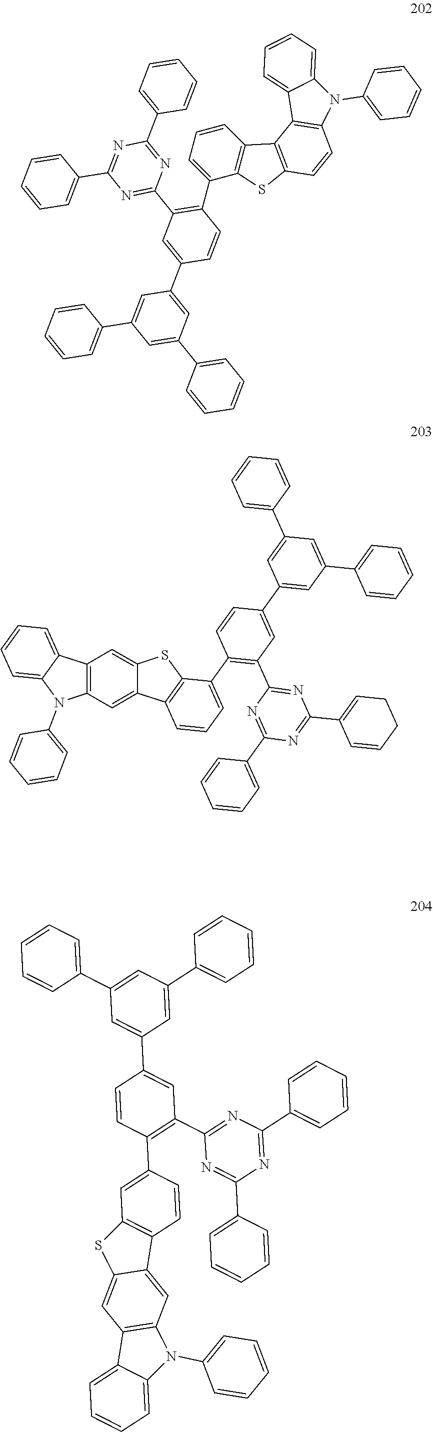 [SMILES: c1ccc(-c2cc(-c3ccccc3)cc(-c3ccc(-c4cccc5c4sc4ccc6c(c7ccccc7n6-c6ccccc6)c45)c(-c4nc(-c5ccccc5)nc(-c5ccccc5)n4)c3)c2)cc1 ; C1=CC(c2nc(-c3ccccc3)nc(-c3cc(-c4cc(-c5ccccc5)cc(-c5ccccc5)c4)ccc3-c3cccc4c3sc3cc5c6ccccc6n(-c6ccccc6)c5cc34)n2)=CCC1 ; c1ccc(-c2cc(-c3ccccc3)cc(-c3ccc(-c4ccc5c(c4)sc4cc6c7ccccc7n(-c7ccccc7)c6cc45)c(-c4nc(-c5ccccc5)nc(-c5ccccc5)n4)c3)c2)cc1]

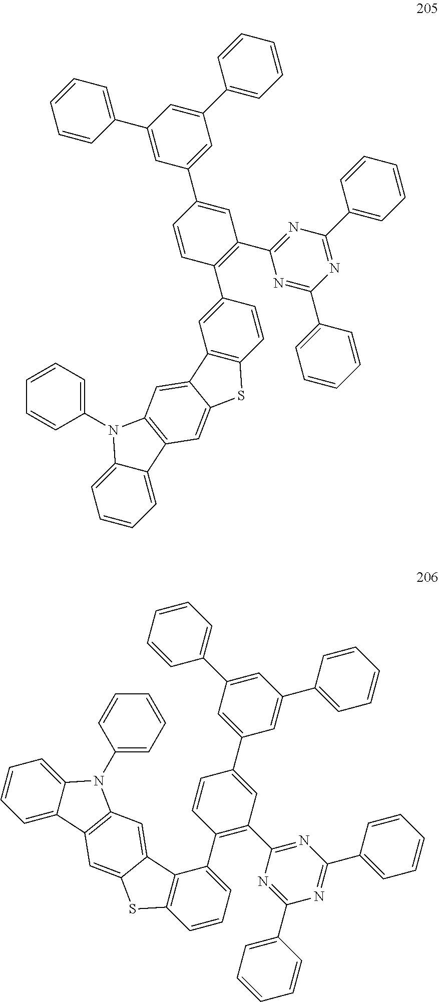 [SMILES: c1ccc(-c2cc(-c3ccccc3)cc(-c3ccc(-c4ccc5sc6cc7c8ccccc8n(-c8ccccc8)c7cc6c5c4)c(-c4nc(-c5ccccc5)nc(-c5ccccc5)n4)c3)c2)cc1 ; c1ccc(-c2cc(-c3ccccc3)cc(-c3ccc(-c4cccc5sc6cc7c8ccccc8n(-c8ccccc8)c7cc6c45)c(-c4nc(-c5ccccc5)nc(-c5ccccc5)n4)c3)c2)cc1]

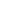

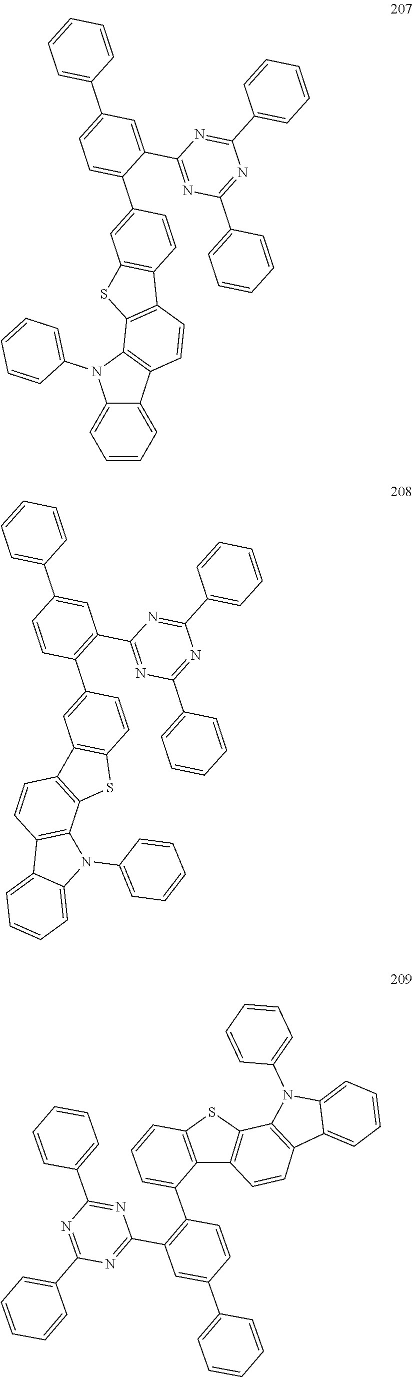 [SMILES: c1ccc(-c2ccc(-c3ccc4c(c3)sc3c4ccc4c5ccccc5n(-c5ccccc5)c43)c(-c3nc(-c4ccccc4)nc(-c4ccccc4)n3)c2)cc1 ; c1ccc(-c2ccc(-c3ccc4sc5c(ccc6c7ccccc7n(-c7ccccc7)c65)c4c3)c(-c3nc(-c4ccccc4)nc(-c4ccccc4)n3)c2)cc1 ; c1ccc(-c2ccc(-c3cccc4sc5c(ccc6c7ccccc7n(-c7ccccc7)c65)c34)c(-c3nc(-c4ccccc4)nc(-c4ccccc4)n3)c2)cc1]

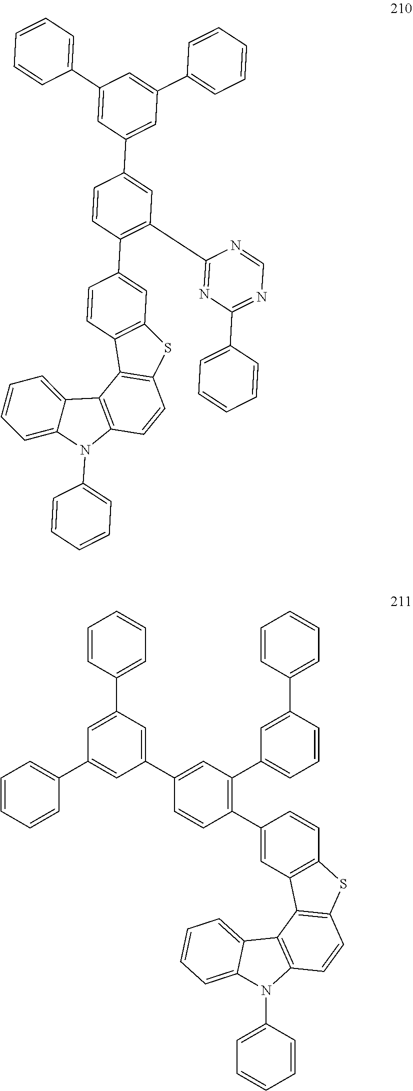 [SMILES: c1ccc(-c2cc(-c3ccccc3)cc(-c3ccc(-c4ccc5c(c4)sc4ccc6c(c7ccccc7n6-c6ccccc6)c45)c(Cc4ncnc(-c5ccccc5)n4)c3)c2)cc1 ; c1ccc(-c2cc(-c3ccccc3)cc(-c3ccc(-c4ccc5sc6ccc7c(c8ccccc8n7-c7ccccc7)c6c5c4)c(-c4cccc(-c5ccccc5)c4)c3)c2)cc1]

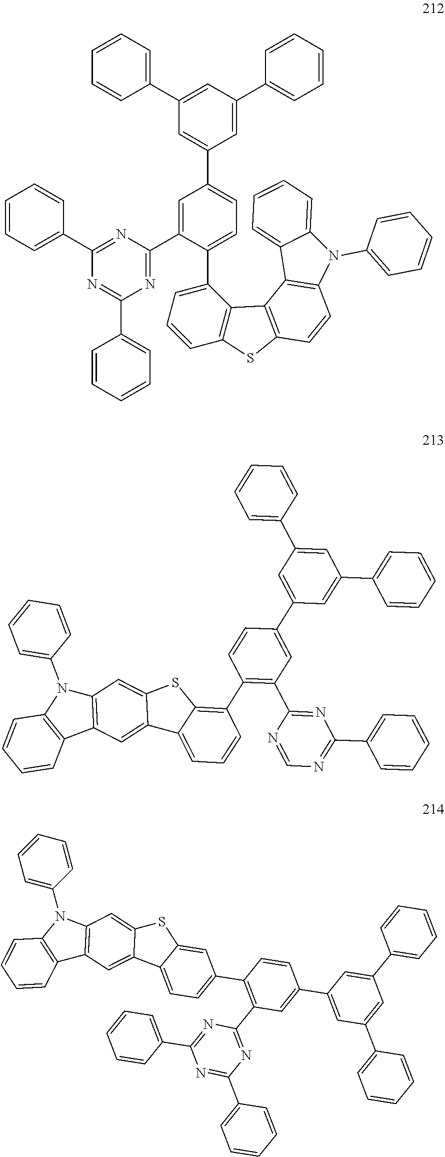 [SMILES: c1ccc(-c2cc(-c3ccccc3)cc(-c3ccc(-c4cccc5sc6ccc7c(c8ccccc8n7-c7ccccc7)c6c45)c(-c4nc(-c5ccccc5)nc(-c5ccccc5)n4)c3)c2)cc1 ; c1ccc(-c2cc(-c3ccccc3)cc(-c3ccc(-c4cccc5c4sc4cc6c(cc45)c4ccccc4n6-c4ccccc4)c(-c4ncnc(-c5ccccc5)n4)c3)c2)cc1 ; c1ccc(-c2cc(-c3ccccc3)cc(-c3ccc(-c4ccc5c(c4)sc4cc6c(cc45)c4ccccc4n6-c4ccccc4)c(-c4nc(-c5ccccc5)nc(-c5ccccc5)n4)c3)c2)cc1]

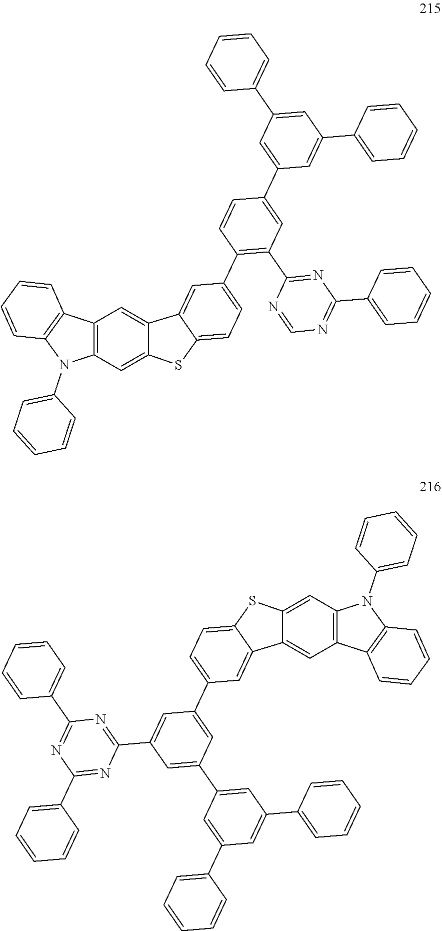 [SMILES: c1ccc(-c2cc(-c3ccccc3)cc(-c3ccc(-c4ccc5sc6cc7c(cc6c5c4)c4ccccc4n7-c4ccccc4)c(-c4ncnc(-c5ccccc5)n4)c3)c2)cc1 ; c1ccc(-c2cc(-c3ccccc3)cc(-c3cc(-c4ccc5sc6cc7c(cc6c5c4)c4ccccc4n7-c4ccccc4)cc(-c4nc(-c5ccccc5)nc(-c5ccccc5)n4)c3)c2)cc1]

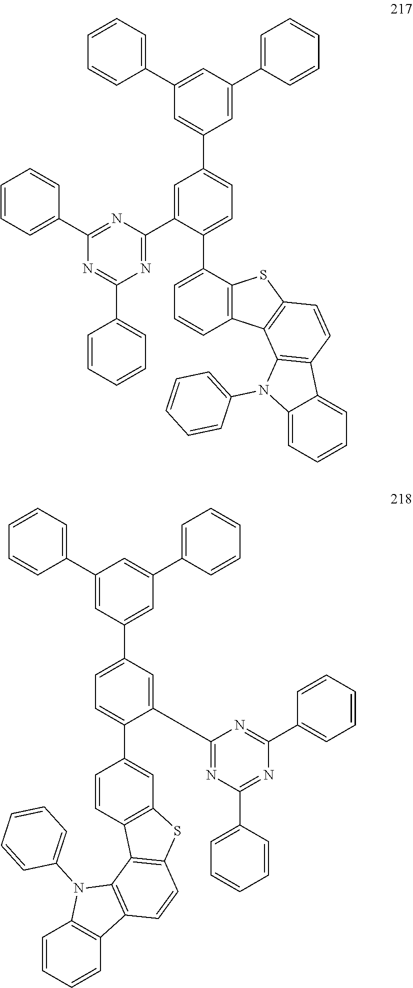 [SMILES: c1ccc(-c2cc(-c3ccccc3)cc(-c3ccc(-c4cccc5c4sc4ccc6c7ccccc7n(-c7ccccc7)c6c45)c(-c4nc(-c5ccccc5)nc(-c5ccccc5)n4)c3)c2)cc1 ; c1ccc(-c2cc(-c3ccccc3)cc(-c3ccc(-c4ccc5c(c4)sc4ccc6c7ccccc7n(-c7ccccc7)c6c45)c(Cc4nc(-c5ccccc5)nc(-c5ccccc5)n4)c3)c2)cc1]

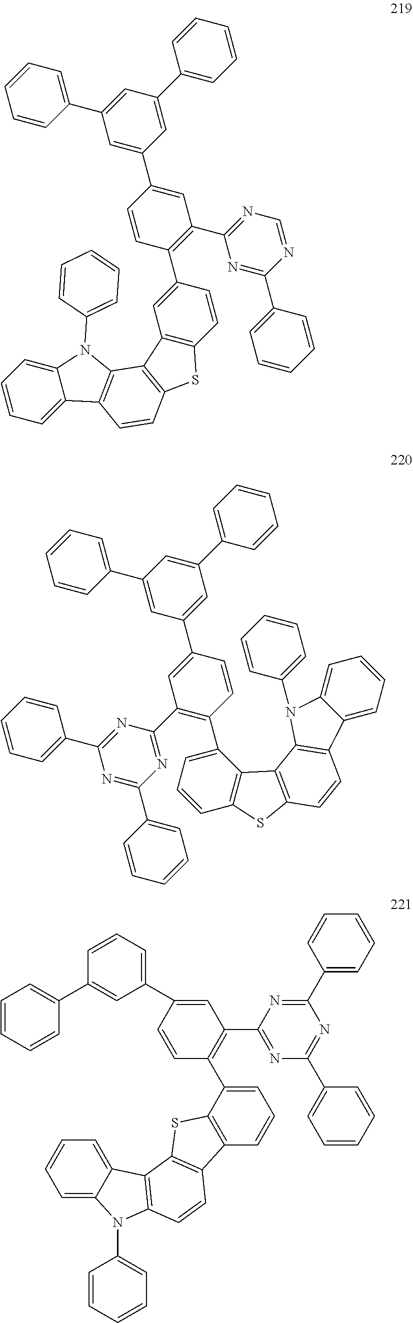 [SMILES: c1ccc(-c2cc(-c3ccccc3)cc(-c3ccc(-c4ccc5sc6ccc7c8ccccc8n(-c8ccccc8)c7c6c5c4)c(-c4ncnc(-c5ccccc5)n4)c3)c2)cc1 ; c1ccc(-c2cc(-c3ccccc3)cc(-c3ccc(-c4cccc5sc6ccc7c8ccccc8n(-c8ccccc8)c7c6c45)c(-c4nc(-c5ccccc5)nc(-c5ccccc5)n4)c3)c2)cc1 ; c1ccc(-c2cccc(-c3ccc(-c4cccc5c4sc4c5ccc5c4c4ccccc4n5-c4ccccc4)c(-c4nc(-c5ccccc5)nc(-c5ccccc5)n4)c3)c2)cc1]

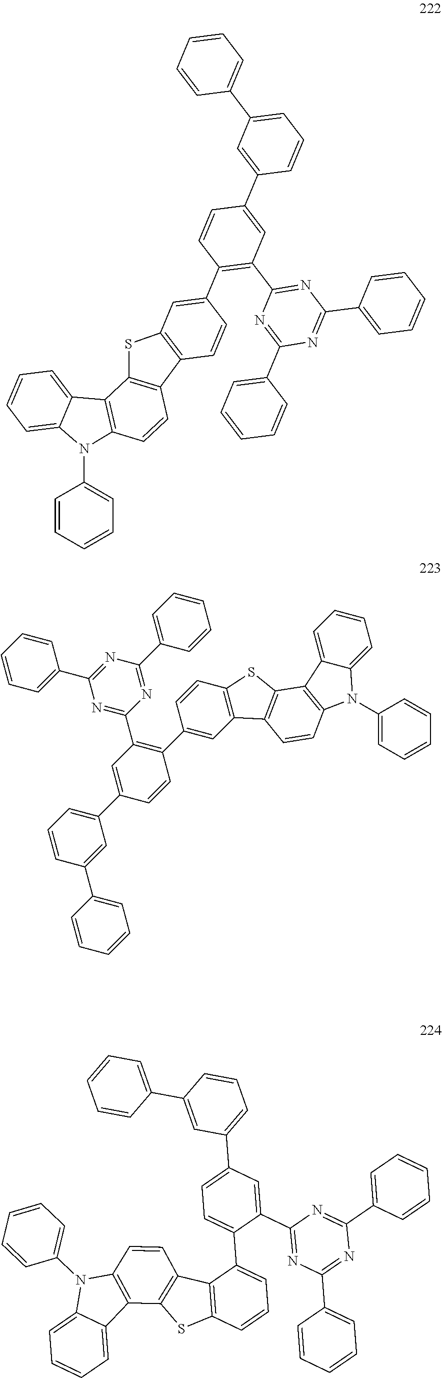 [SMILES: c1ccc(-c2cccc(-c3ccc(-c4ccc5c(c4)sc4c5ccc5c4c4ccccc4n5-c4ccccc4)c(-c4nc(-c5ccccc5)nc(-c5ccccc5)n4)c3)c2)cc1 ; c1ccc(-c2cccc(-c3ccc(-c4ccc5sc6c(ccc7c6c6ccccc6n7-c6ccccc6)c5c4)c(-c4nc(-c5ccccc5)nc(-c5ccccc5)n4)c3)c2)cc1 ; c1ccc(-c2cccc(-c3ccc(-c4cccc5sc6c(ccc7c6c6ccccc6n7-c6ccccc6)c45)c(-c4nc(-c5ccccc5)nc(-c5ccccc5)n4)c3)c2)cc1]

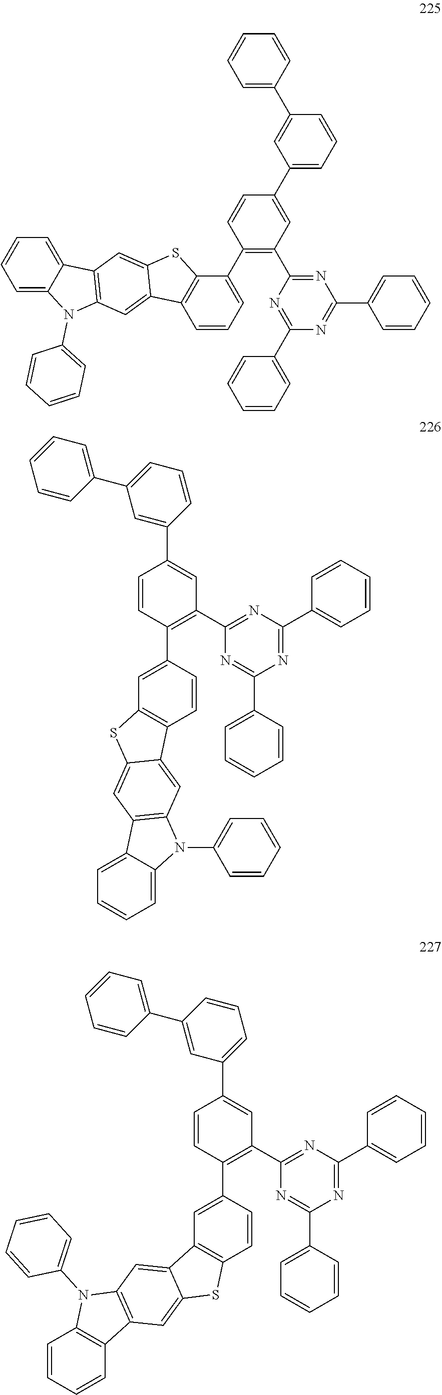

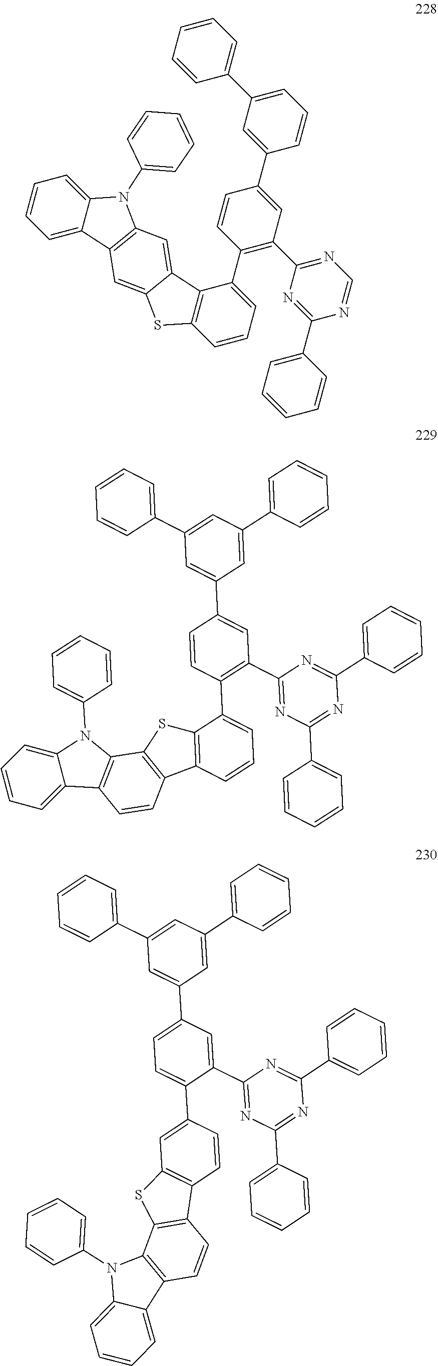

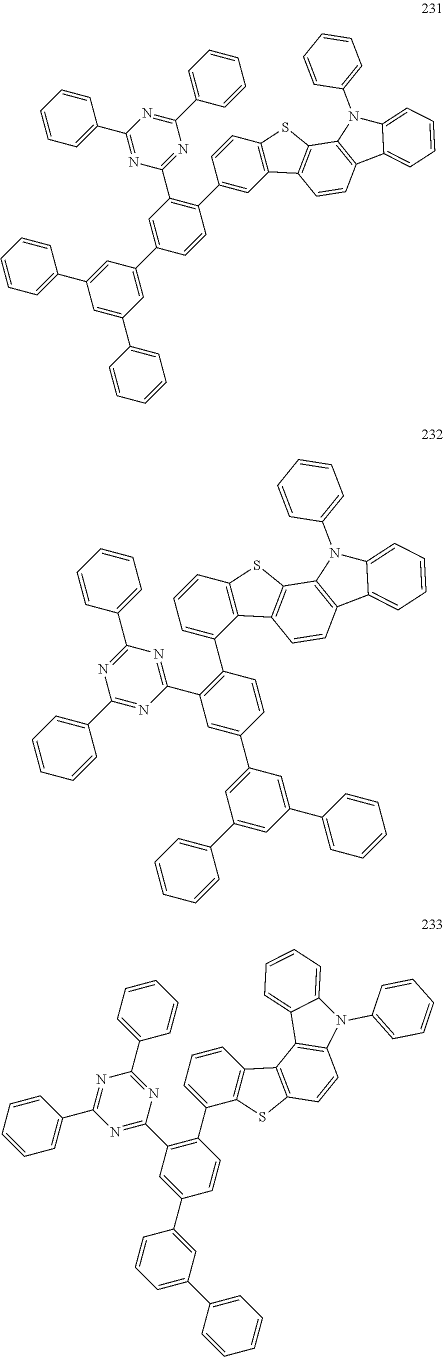 [SMILES: c1ccc(-c2cc(-c3ccccc3)cc(-c3ccc(-c4ccc5sc6c(ccc7c8ccccc8n(-c8ccccc8)c76)c5c4)c(-c4nc(-c5ccccc5)nc(-c5ccccc5)n4)c3)c2)cc1 ; c1ccc(-c2cc(-c3ccccc3)cc(-c3ccc(-c4cccc5sc6c(ccc7c8ccccc8n(-c8ccccc8)c76)c45)c(-c4nc(-c5ccccc5)nc(-c5ccccc5)n4)c3)c2)cc1 ; c1ccc(-c2cccc(-c3ccc(-c4cccc5c4sc4ccc6c(c7ccccc7n6-c6ccccc6)c45)c(-c4nc(-c5ccccc5)nc(-c5ccccc5)n4)c3)c2)cc1]

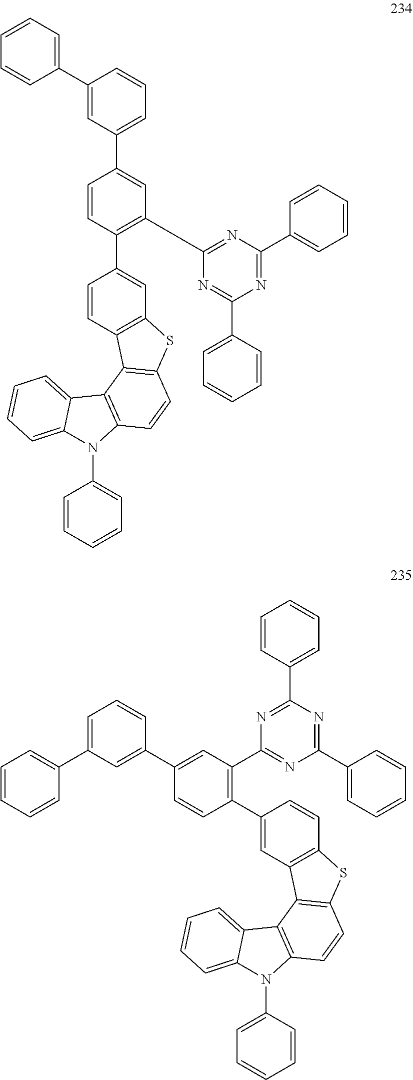 [SMILES: c1ccc(-c2cccc(-c3ccc(-c4ccc5c(c4)sc4ccc6c(c7ccccc7n6-c6ccccc6)c45)c(Cc4nc(-c5ccccc5)nc(-c5ccccc5)n4)c3)c2)cc1 ; c1ccc(-c2cccc(-c3ccc(-c4ccc5sc6ccc7c(c8ccccc8n7-c7ccccc7)c6c5c4)c(-c4nc(-c5ccccc5)nc(-c5ccccc5)n4)c3)c2)cc1]

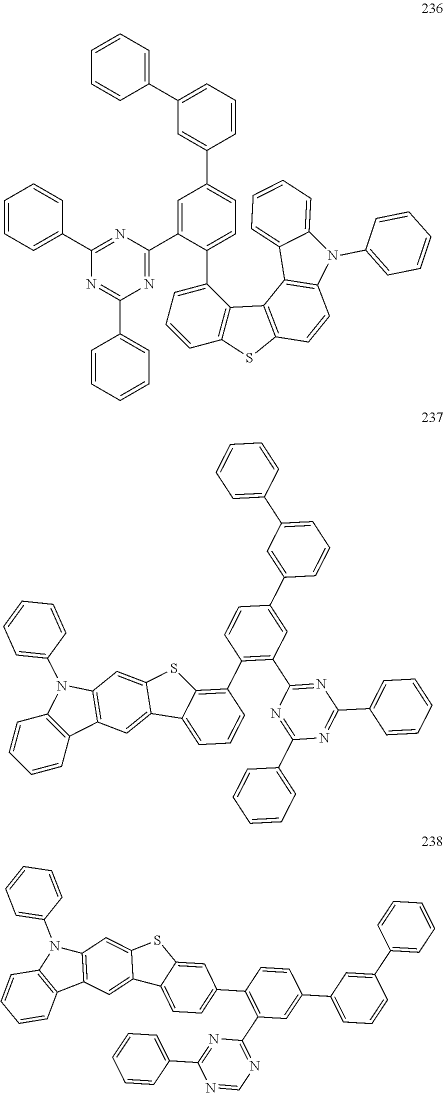 [SMILES: c1ccc(-c2cccc(-c3ccc(-c4cccc5sc6ccc7c(c8ccccc8n7-c7ccccc7)c6c45)c(-c4nc(-c5ccccc5)nc(-c5ccccc5)n4)c3)c2)cc1 ; c1ccc(-c2cccc(-c3ccc(-c4cccc5c4sc4cc6c(cc45)c4ccccc4n6-c4ccccc4)c(-c4nc(-c5ccccc5)nc(-c5ccccc5)n4)c3)c2)cc1 ; c1ccc(-c2cccc(-c3ccc(-c4ccc5c(c4)sc4cc6c(cc45)c4ccccc4n6-c4ccccc4)c(-c4ncnc(-c5ccccc5)n4)c3)c2)cc1]

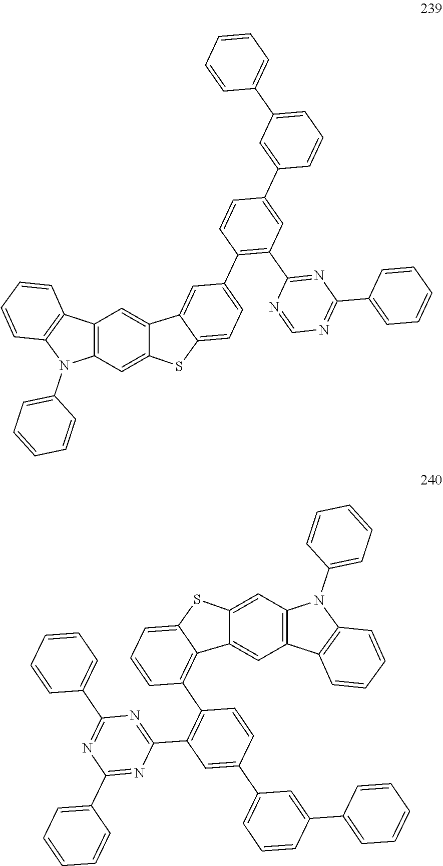 [SMILES: c1ccc(-c2cccc(-c3ccc(-c4ccc5sc6cc7c(cc6c5c4)c4ccccc4n7-c4ccccc4)c(-c4ncnc(-c5ccccc5)n4)c3)c2)cc1 ; c1ccc(-c2cccc(-c3ccc(-c4cccc5sc6cc7c(cc6c45)c4ccccc4n7-c4ccccc4)c(-c4nc(-c5ccccc5)nc(-c5ccccc5)n4)c3)c2)cc1]

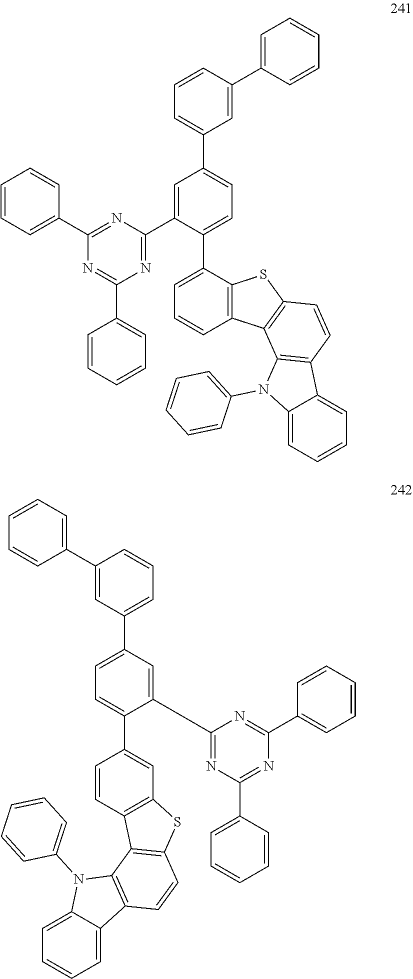 [SMILES: c1ccc(-c2cccc(-c3ccc(-c4cccc5c4sc4ccc6c7ccccc7n(-c7ccccc7)c6c45)c(-c4nc(-c5ccccc5)nc(-c5ccccc5)n4)c3)c2)cc1 ; c1ccc(-c2cccc(-c3ccc(-c4ccc5c(c4)sc4ccc6c7ccccc7n(-c7ccccc7)c6c45)c(Cc4nc(-c5ccccc5)nc(-c5ccccc5)n4)c3)c2)cc1]

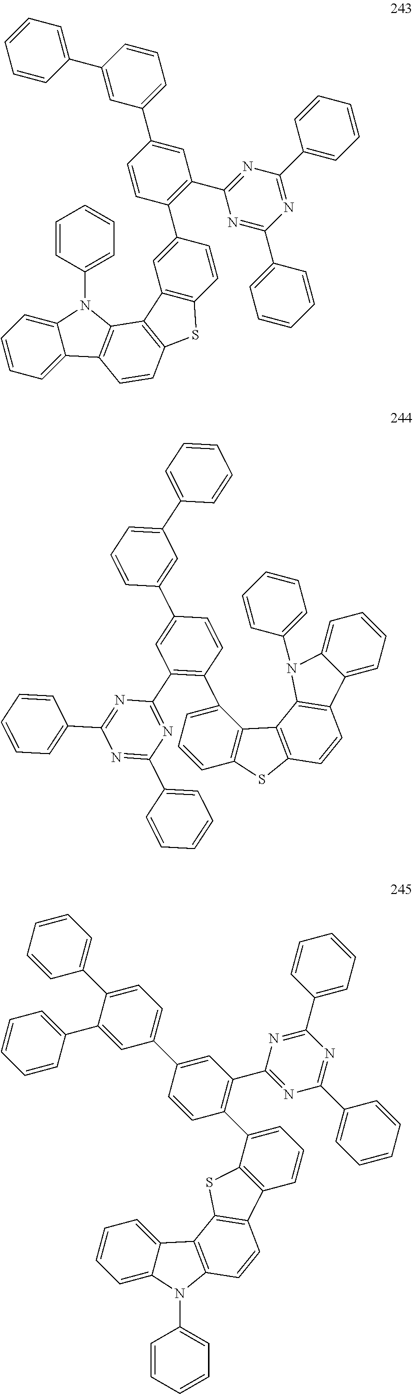 [SMILES: c1ccc(-c2cccc(-c3ccc(-c4ccc5sc6ccc7c8ccccc8n(-c8ccccc8)c7c6c5c4)c(-c4nc(-c5ccccc5)nc(-c5ccccc5)n4)c3)c2)cc1 ; c1ccc(-c2cccc(-c3ccc(-c4cccc5sc6ccc7c8ccccc8n(-c8ccccc8)c7c6c45)c(-c4nc(-c5ccccc5)nc(-c5ccccc5)n4)c3)c2)cc1 ; c1ccc(-c2nc(-c3ccccc3)nc(-c3cc(-c4ccc(-c5ccccc5)c(-c5ccccc5)c4)ccc3-c3cccc4c3sc3c4ccc4c3c3ccccc3n4-c3ccccc3)n2)cc1]

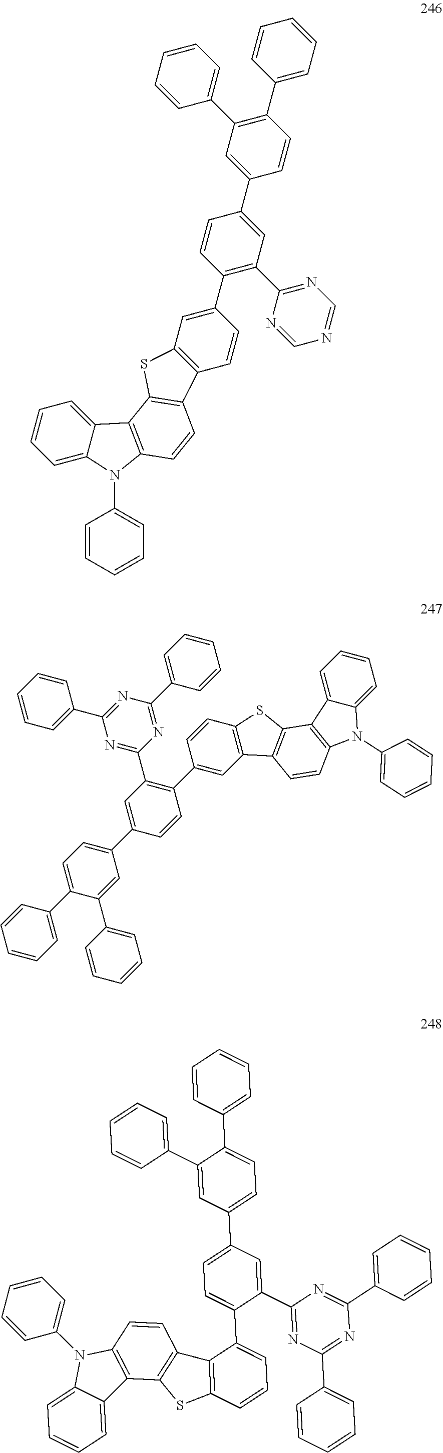 [SMILES: c1ccc(-c2ccc(-c3ccc(-c4ccc5c(c4)sc4c5ccc5c4c4ccccc4n5-c4ccccc4)c(-c4ncncn4)c3)cc2-c2ccccc2)cc1 ; c1ccc(-c2nc(-c3ccccc3)nc(-c3cc(-c4ccc(-c5ccccc5)c(-c5ccccc5)c4)ccc3-c3ccc4sc5c(ccc6c5c5ccccc5n6-c5ccccc5)c4c3)n2)cc1 ; c1ccc(-c2nc(-c3ccccc3)nc(-c3cc(-c4ccc(-c5ccccc5)c(-c5ccccc5)c4)ccc3-c3cccc4sc5c(ccc6c5c5ccccc5n6-c5ccccc5)c34)n2)cc1]

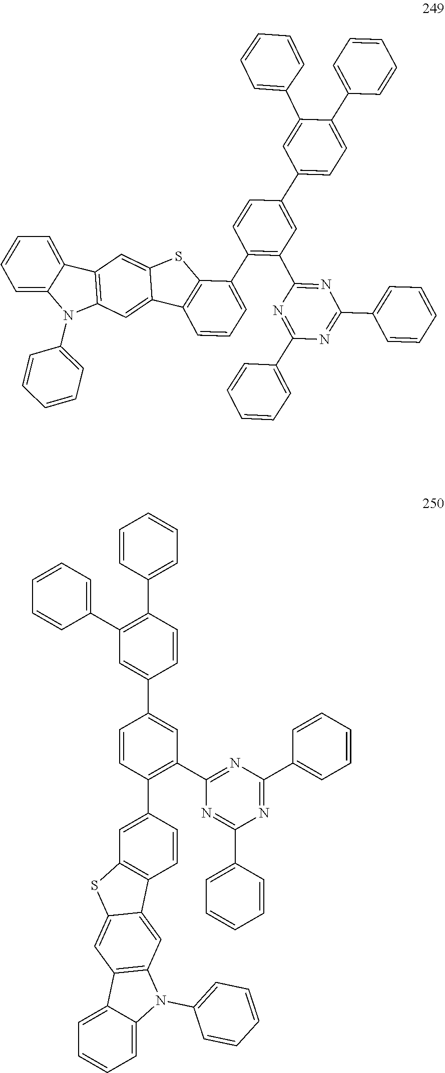 [SMILES: c1ccc(-c2nc(-c3ccccc3)nc(-c3cc(-c4ccc(-c5ccccc5)c(-c5ccccc5)c4)ccc3-c3cccc4c3sc3cc5c6ccccc6n(-c6ccccc6)c5cc34)n2)cc1 ; c1ccc(-c2nc(-c3ccccc3)nc(-c3cc(-c4ccc(-c5ccccc5)c(-c5ccccc5)c4)ccc3-c3ccc4c(c3)sc3cc5c6ccccc6n(-c6ccccc6)c5cc34)n2)cc1]

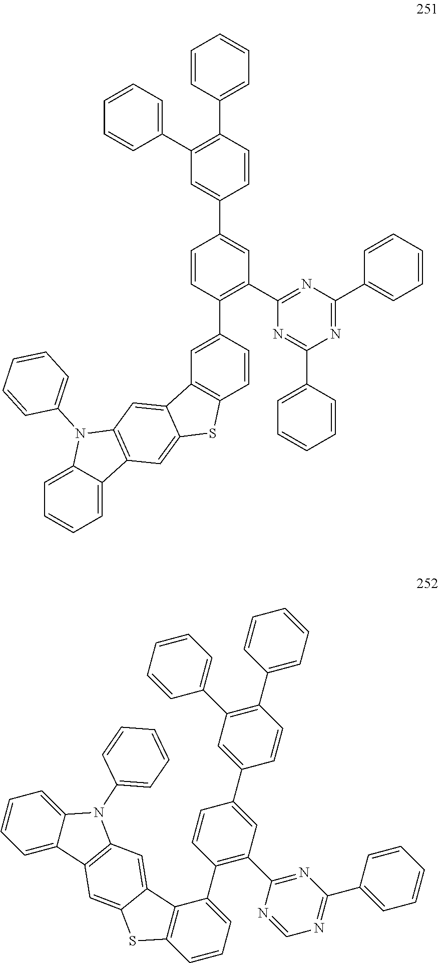 [SMILES: c1ccc(-c2nc(-c3ccccc3)nc(-c3cc(-c4ccc(-c5ccccc5)c(-c5ccccc5)c4)ccc3-c3ccc4sc5cc6c7ccccc7n(-c7ccccc7)c6cc5c4c3)n2)cc1 ; c1ccc(-c2ncnc(-c3cc(-c4ccc(-c5ccccc5)c(-c5ccccc5)c4)ccc3-c3cccc4sc5cc6c7ccccc7n(-c7ccccc7)c6cc5c34)n2)cc1]

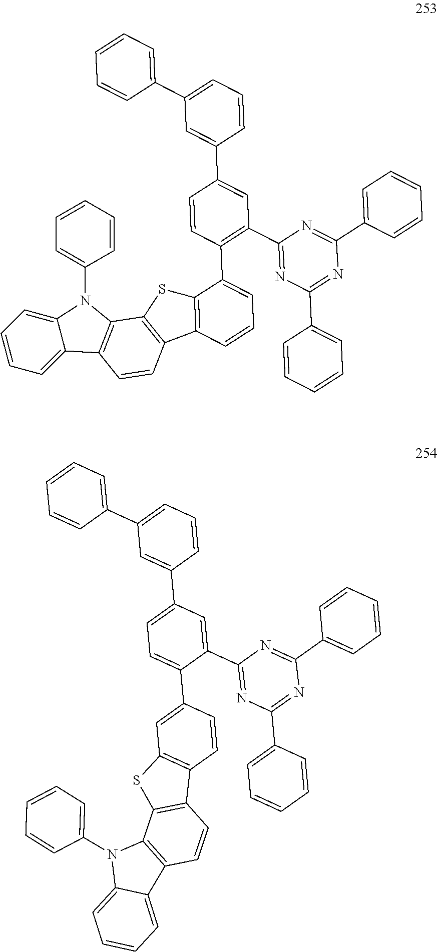 [SMILES: c1ccc(-c2cccc(-c3ccc(-c4cccc5c4sc4c5ccc5c6ccccc6n(-c6ccccc6)c54)c(-c4nc(-c5ccccc5)nc(-c5ccccc5)n4)c3)c2)cc1 ; c1ccc(-c2cccc(-c3ccc(-c4ccc5c(c4)sc4c5ccc5c6ccccc6n(-c6ccccc6)c54)c(-c4nc(-c5ccccc5)nc(-c5ccccc5)n4)c3)c2)cc1]

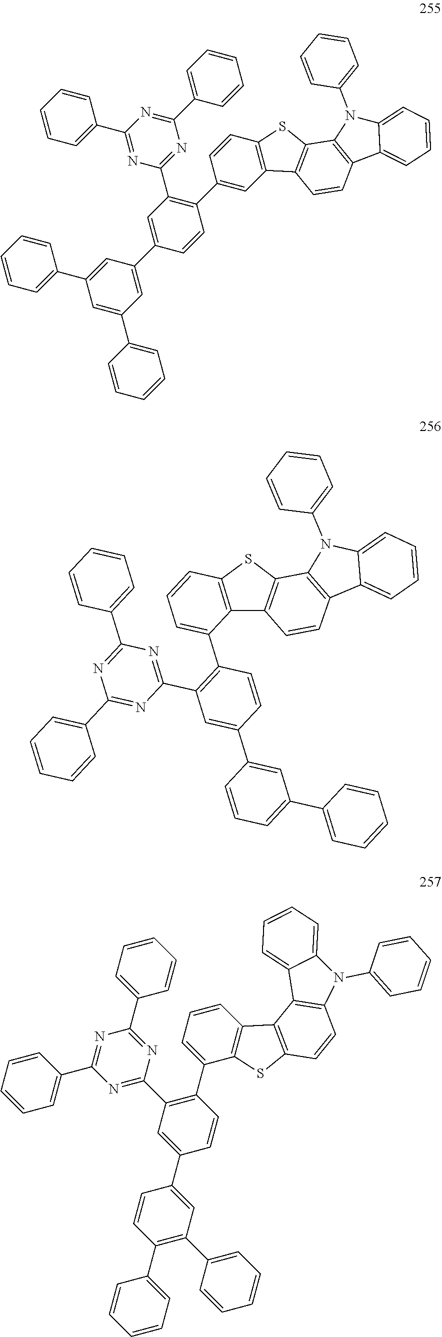 [SMILES: c1ccc(-c2cc(-c3ccccc3)cc(-c3ccc(-c4ccc5sc6c(ccc7c8ccccc8n(-c8ccccc8)c76)c5c4)c(-c4nc(-c5ccccc5)nc(-c5ccccc5)n4)c3)c2)cc1 ; c1ccc(-c2cccc(-c3ccc(-c4cccc5sc6c(ccc7c8ccccc8n(-c8ccccc8)c76)c45)c(-c4nc(-c5ccccc5)nc(-c5ccccc5)n4)c3)c2)cc1 ; c1ccc(-c2nc(-c3ccccc3)nc(-c3cc(-c4ccc(-c5ccccc5)c(-c5ccccc5)c4)ccc3-c3cccc4c3sc3ccc5c(c6ccccc6n5-c5ccccc5)c34)n2)cc1]

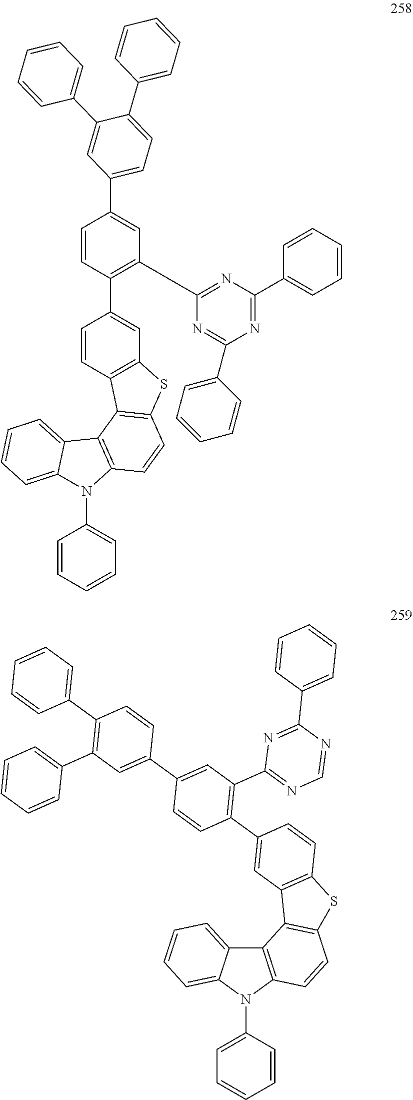 [SMILES: c1ccc(-c2nc(Cc3cc(-c4ccc(-c5ccccc5)c(-c5ccccc5)c4)ccc3-c3ccc4c(c3)sc3ccc5c(c6ccccc6n5-c5ccccc5)c34)nc(-c3ccccc3)n2)cc1 ; c1ccc(-c2ncnc(-c3cc(-c4ccc(-c5ccccc5)c(-c5ccccc5)c4)ccc3-c3ccc4sc5ccc6c(c7ccccc7n6-c6ccccc6)c5c4c3)n2)cc1]

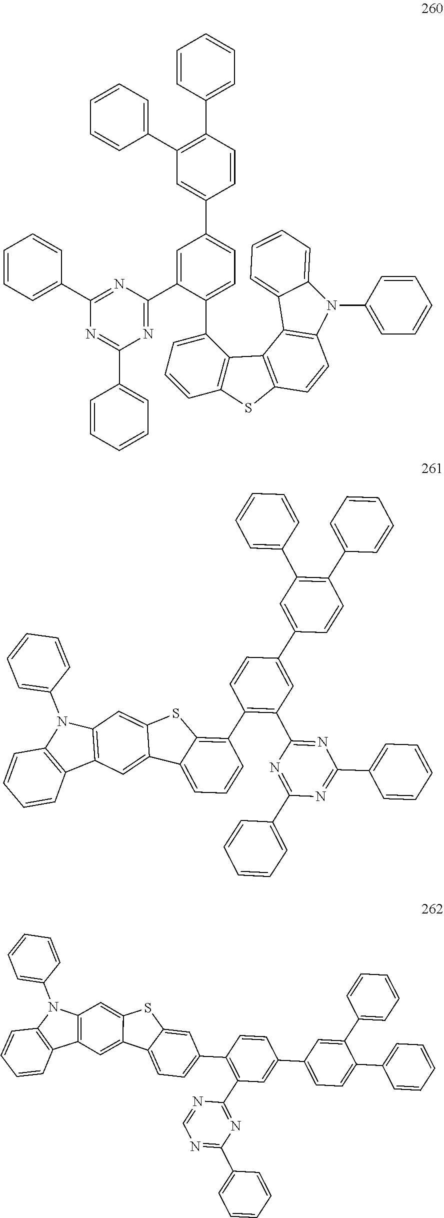 [SMILES: c1ccc(-c2nc(-c3ccccc3)nc(-c3cc(-c4ccc(-c5ccccc5)c(-c5ccccc5)c4)ccc3-c3cccc4sc5ccc6c(c7ccccc7n6-c6ccccc6)c5c34)n2)cc1 ; c1ccc(-c2nc(-c3ccccc3)nc(-c3cc(-c4ccc(-c5ccccc5)c(-c5ccccc5)c4)ccc3-c3cccc4c3sc3cc5c(cc34)c3ccccc3n5-c3ccccc3)n2)cc1 ; c1ccc(-c2ncnc(-c3cc(-c4ccc(-c5ccccc5)c(-c5ccccc5)c4)ccc3-c3ccc4c(c3)sc3cc5c(cc34)c3ccccc3n5-c3ccccc3)n2)cc1]

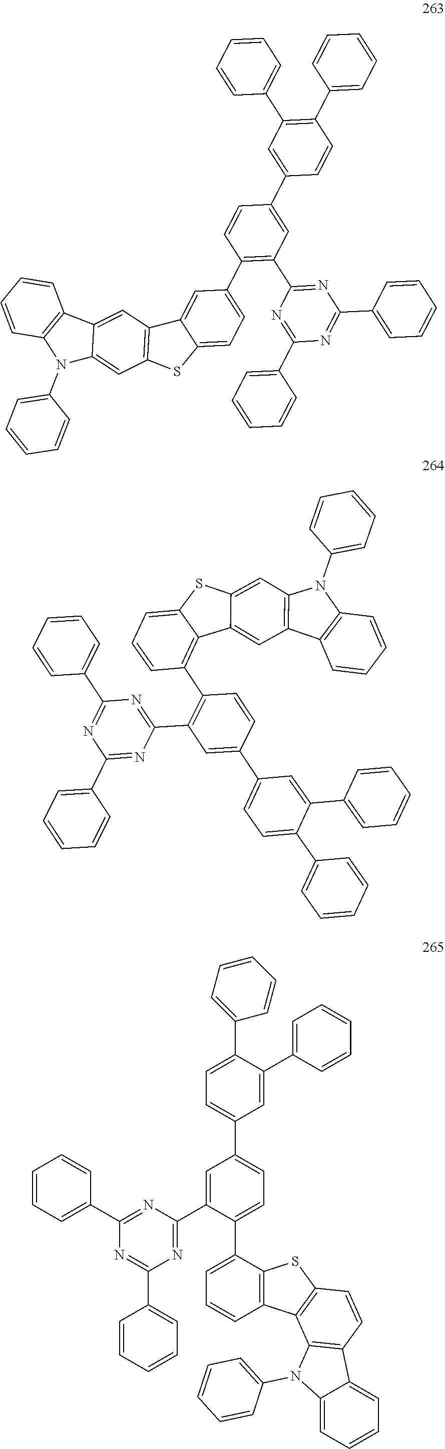 [SMILES: c1ccc(-c2nc(-c3ccccc3)nc(-c3cc(-c4ccc(-c5ccccc5)c(-c5ccccc5)c4)ccc3-c3ccc4sc5cc6c(cc5c4c3)c3ccccc3n6-c3ccccc3)n2)cc1 ; c1ccc(-c2nc(-c3ccccc3)nc(-c3cc(-c4ccc(-c5ccccc5)c(-c5ccccc5)c4)ccc3-c3cccc4sc5cc6c(cc5c34)c3ccccc3n6-c3ccccc3)n2)cc1 ; c1ccc(-c2nc(-c3ccccc3)nc(-c3cc(-c4ccc(-c5ccccc5)c(-c5ccccc5)c4)ccc3-c3cccc4c3sc3ccc5c6ccccc6n(-c6ccccc6)c5c34)n2)cc1]

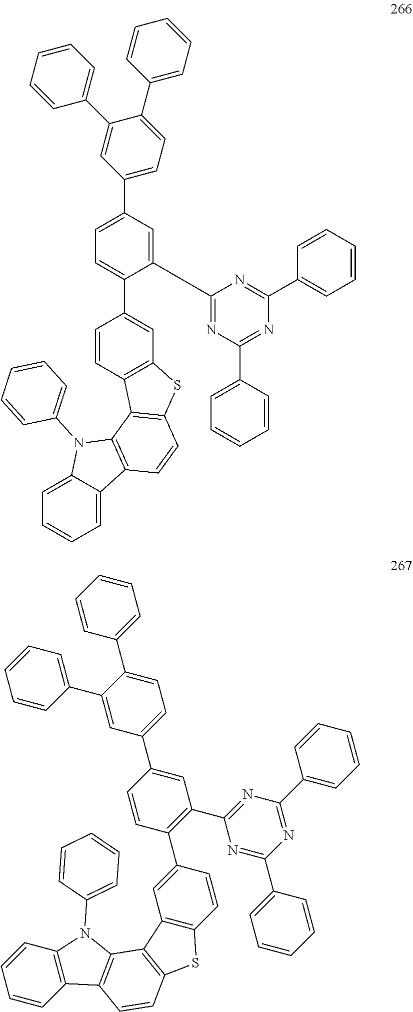

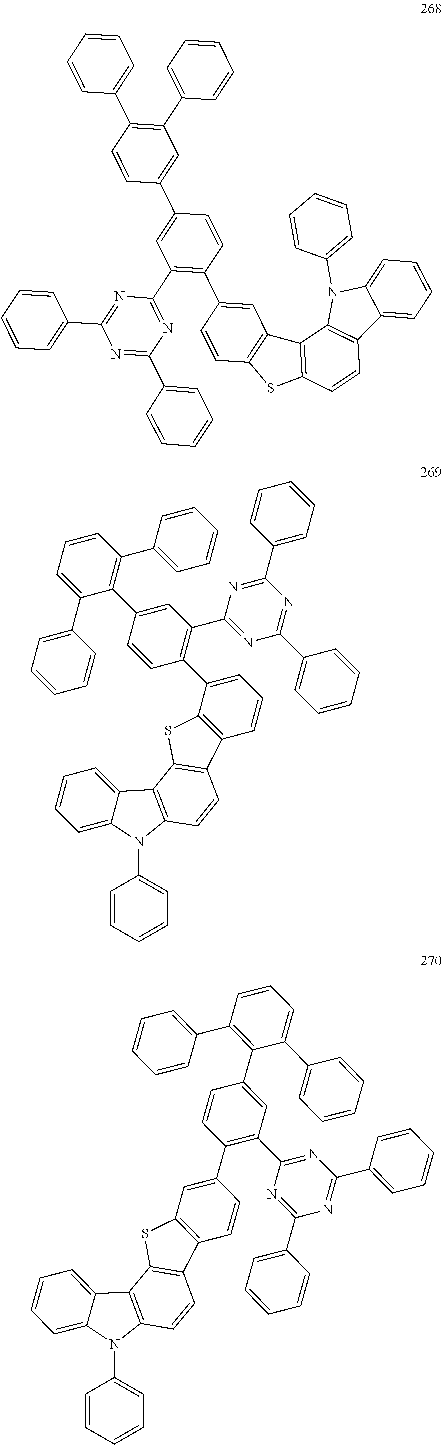 [SMILES: c1ccc(-c2nc(-c3ccccc3)nc(-c3cc(-c4ccc(-c5ccccc5)c(-c5ccccc5)c4)ccc3-c3ccc4sc5ccc6c7ccccc7n(-c7ccccc7)c6c5c4c3)n2)cc1 ; c1ccc(-c2nc(-c3ccccc3)nc(-c3cc(-c4c(-c5ccccc5)cccc4-c4ccccc4)ccc3-c3cccc4c3sc3c4ccc4c3c3ccccc3n4-c3ccccc3)n2)cc1 ; c1ccc(-c2nc(-c3ccccc3)nc(-c3cc(-c4c(-c5ccccc5)cccc4-c4ccccc4)ccc3-c3ccc4c(c3)sc3c4ccc4c3c3ccccc3n4-c3ccccc3)n2)cc1]

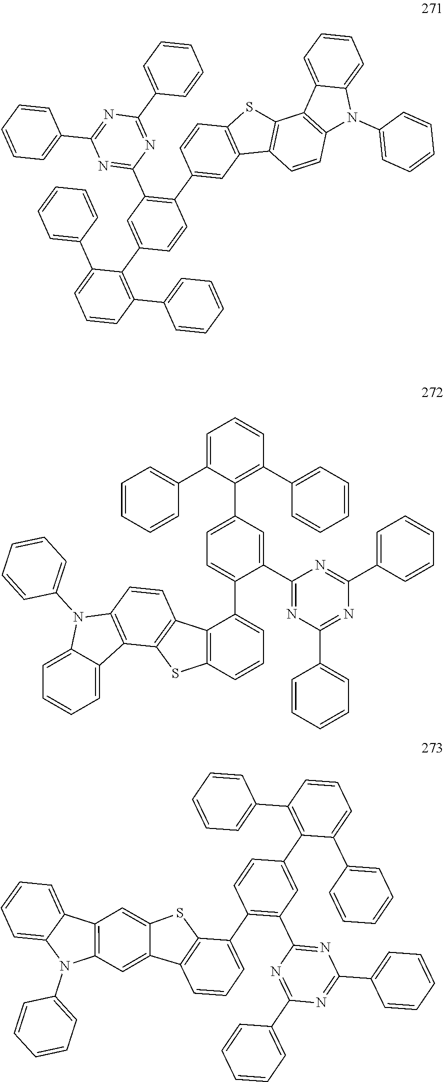 [SMILES: c1ccc(-c2nc(-c3ccccc3)nc(-c3cc(-c4c(-c5ccccc5)cccc4-c4ccccc4)ccc3-c3ccc4sc5c(ccc6c5c5ccccc5n6-c5ccccc5)c4c3)n2)cc1 ; c1ccc(-c2nc(-c3ccccc3)nc(-c3cc(-c4c(-c5ccccc5)cccc4-c4ccccc4)ccc3-c3cccc4sc5c(ccc6c5c5ccccc5n6-c5ccccc5)c34)n2)cc1 ; c1ccc(-c2nc(-c3ccccc3)nc(-c3cc(-c4c(-c5ccccc5)cccc4-c4ccccc4)ccc3-c3cccc4c3sc3cc5c6ccccc6n(-c6ccccc6)c5cc34)n2)cc1]

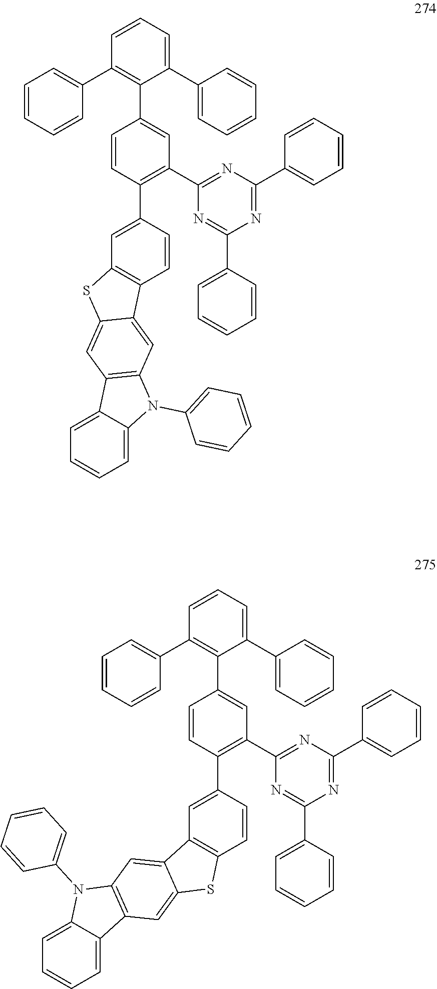 [SMILES: c1ccc(-c2nc(-c3ccccc3)nc(-c3cc(-c4c(-c5ccccc5)cccc4-c4ccccc4)ccc3-c3ccc4c(c3)sc3cc5c6ccccc6n(-c6ccccc6)c5cc34)n2)cc1 ; c1ccc(-c2nc(-c3ccccc3)nc(-c3cc(-c4c(-c5ccccc5)cccc4-c4ccccc4)ccc3-c3ccc4sc5cc6c7ccccc7n(-c7ccccc7)c6cc5c4c3)n2)cc1]

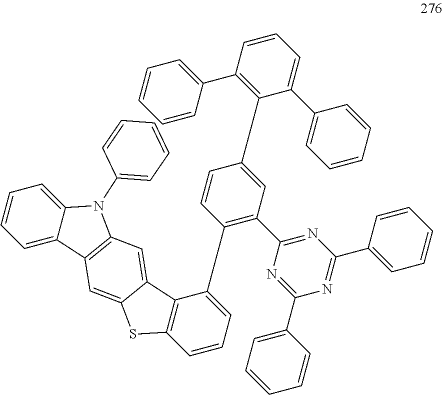 [SMILES: c1ccc(-c2nc(-c3ccccc3)nc(-c3cc(Cc4c(-c5ccccc5)cccc4-c4ccccc4)ccc3Cc3cccc4sc5cc6c7ccccc7n(-c7ccccc7)c6cc5c34)n2)cc1]

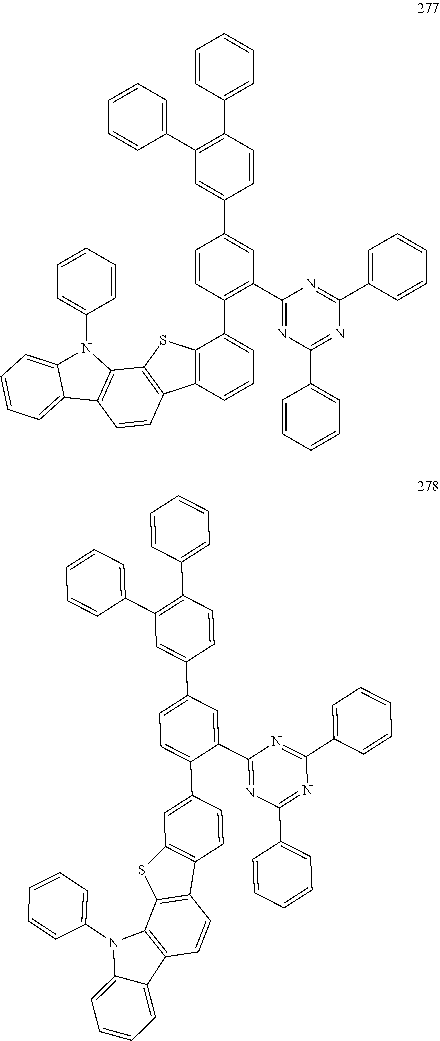 [SMILES: c1ccc(-c2nc(-c3ccccc3)nc(-c3cc(-c4ccc(-c5ccccc5)c(-c5ccccc5)c4)ccc3-c3cccc4c3sc3c4ccc4c5ccccc5n(-c5ccccc5)c43)n2)cc1 ; c1ccc(-c2nc(-c3ccccc3)nc(-c3cc(-c4ccc(-c5ccccc5)c(-c5ccccc5)c4)ccc3-c3ccc4c(c3)sc3c4ccc4c5ccccc5n(-c5ccccc5)c43)n2)cc1]

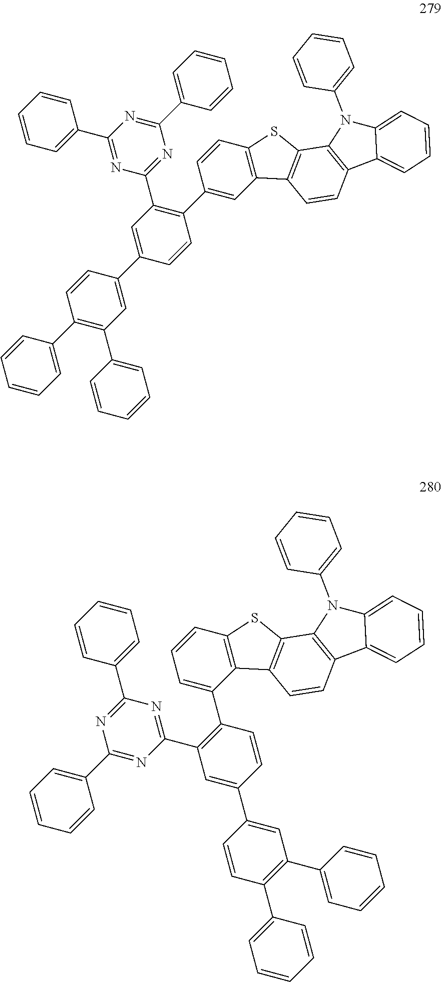 [SMILES: c1ccc(-c2nc(-c3ccccc3)nc(-c3cc(-c4ccc(-c5ccccc5)c(-c5ccccc5)c4)ccc3-c3ccc4sc5c(ccc6c7ccccc7n(-c7ccccc7)c65)c4c3)n2)cc1 ; c1ccc(-c2nc(-c3ccccc3)nc(-c3cc(-c4ccc(-c5ccccc5)c(-c5ccccc5)c4)ccc3-c3cccc4sc5c(ccc6c7ccccc7n(-c7ccccc7)c65)c34)n2)cc1]

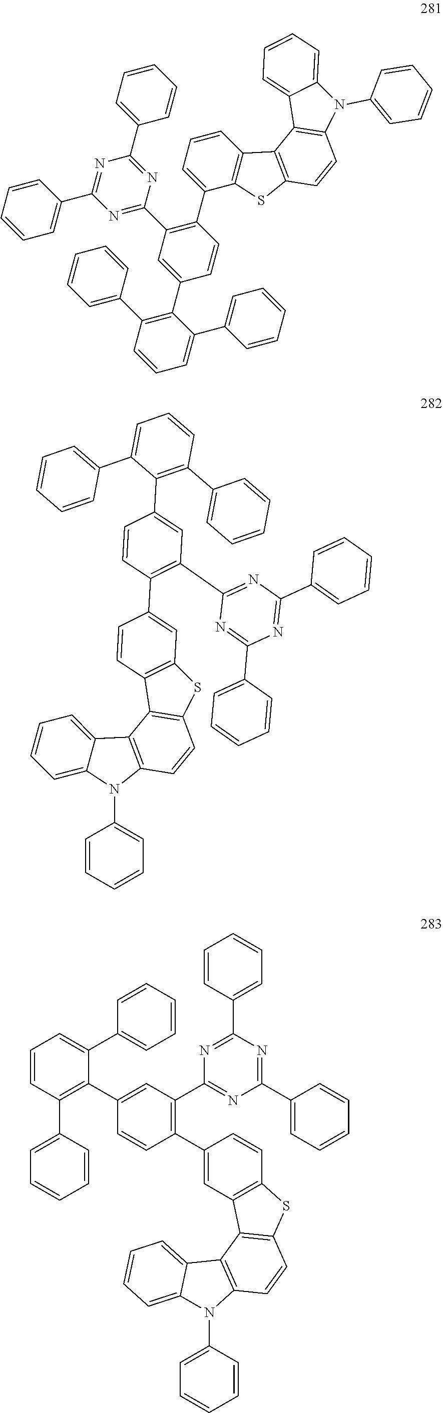

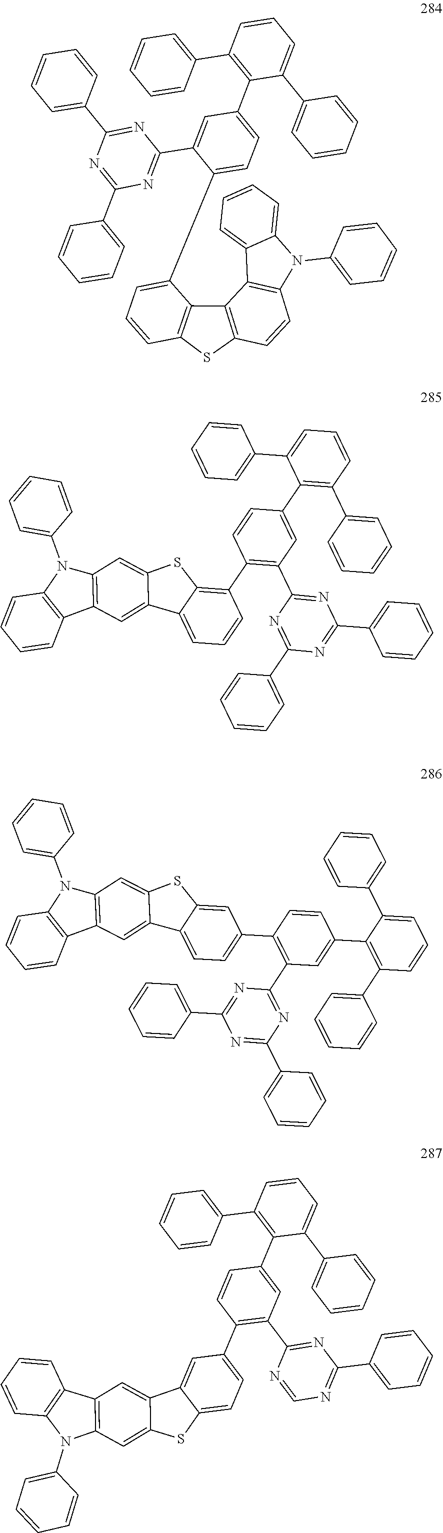 [SMILES: c1ccc(-c2nc(-c3ccccc3)nc(-c3cc(-c4c(-c5ccccc5)cccc4-c4ccccc4)ccc3-c3cccc4sc5ccc6c(c7ccccc7n6-c6ccccc6)c5c34)n2)cc1 ; c1ccc(-c2nc(-c3ccccc3)nc(-c3cc(-c4c(-c5ccccc5)cccc4-c4ccccc4)ccc3-c3cccc4c3sc3cc5c(cc34)c3ccccc3n5-c3ccccc3)n2)cc1 ; c1ccc(-c2nc(-c3ccccc3)nc(-c3cc(-c4c(-c5ccccc5)cccc4-c4ccccc4)ccc3-c3ccc4c(c3)sc3cc5c(cc34)c3ccccc3n5-c3ccccc3)n2)cc1 ; c1ccc(-c2ncnc(-c3cc(-c4c(-c5ccccc5)cccc4-c4ccccc4)ccc3-c3ccc4sc5cc6c(cc5c4c3)c3ccccc3n6-c3ccccc3)n2)cc1]

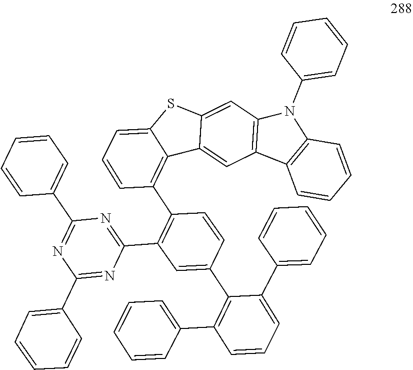 [SMILES: c1ccc(-c2nc(-c3ccccc3)nc(-c3cc(-c4c(-c5ccccc5)cccc4-c4ccccc4)ccc3-c3cccc4sc5cc6c(cc5c34)c3ccccc3n6-c3ccccc3)n2)cc1]

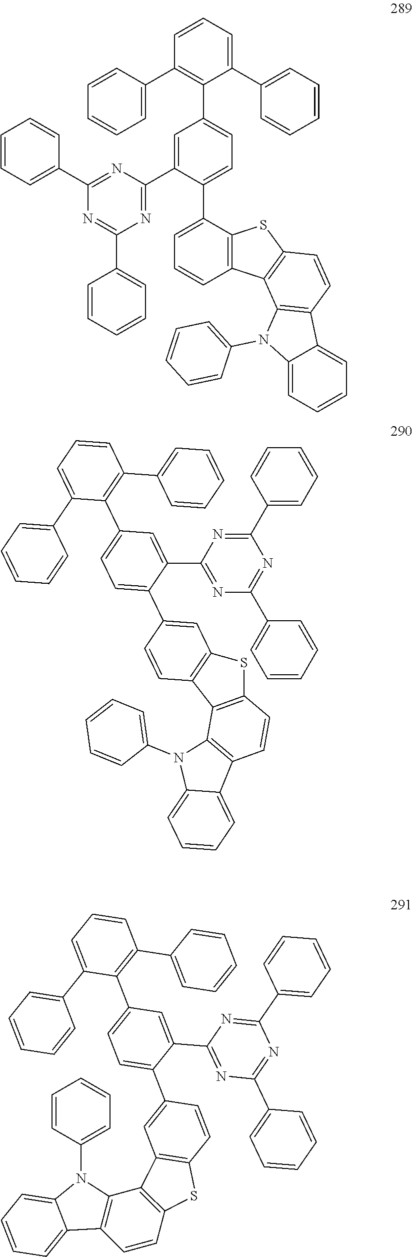

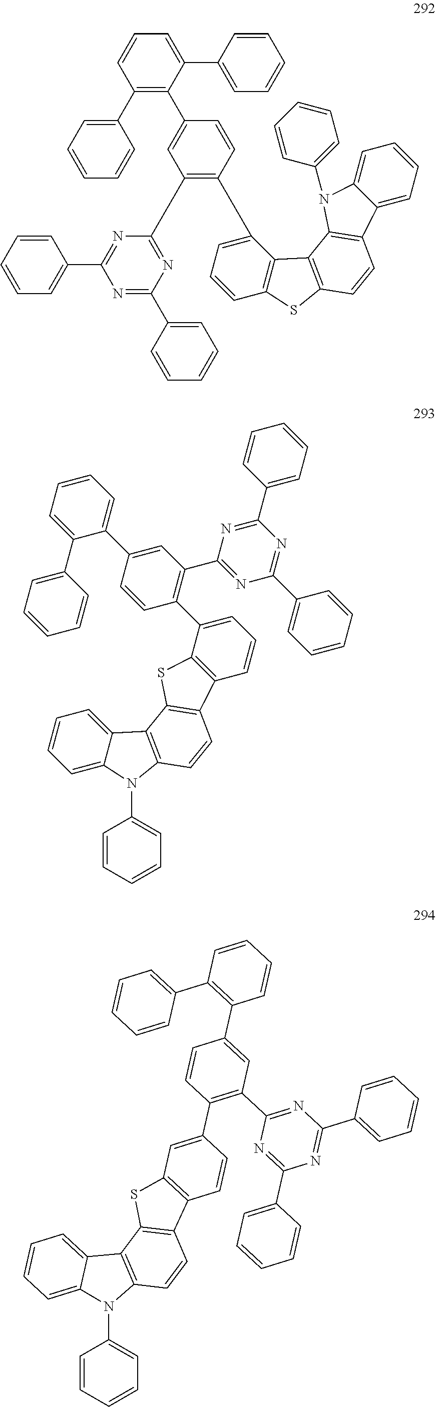 [SMILES: c1ccc(-c2nc(Cc3cc(-c4c(-c5ccccc5)cccc4-c4ccccc4)ccc3Cc3cccc4sc5ccc6c7ccccc7n(-c7ccccc7)c6c5c34)nc(-c3ccccc3)n2)cc1 ; c1ccc(-c2nc(-c3ccccc3)nc(-c3cc(-c4ccccc4-c4ccccc4)ccc3-c3cccc4c3sc3c4ccc4c3c3ccccc3n4-c3ccccc3)n2)cc1 ; c1ccc(-c2nc(-c3ccccc3)nc(-c3cc(-c4ccccc4-c4ccccc4)ccc3-c3ccc4c(c3)sc3c4ccc4c3c3ccccc3n4-c3ccccc3)n2)cc1]

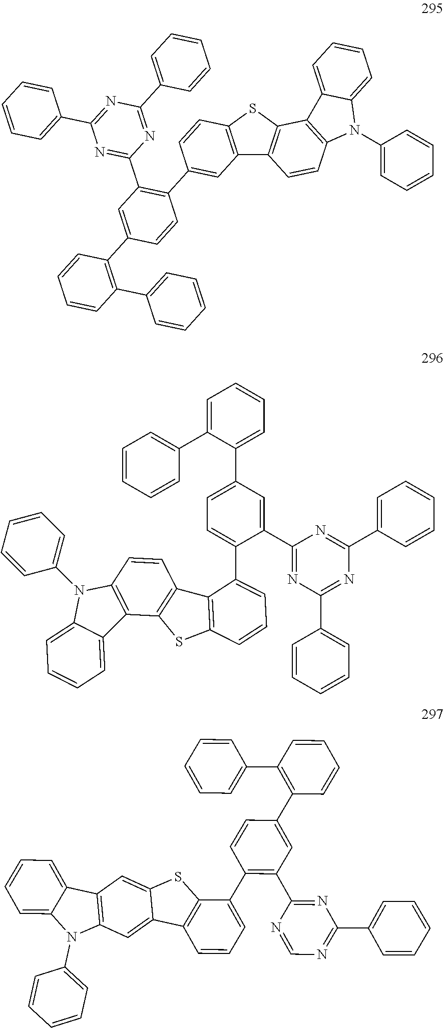 [SMILES: c1ccc(-c2nc(-c3ccccc3)nc(-c3cc(-c4ccccc4-c4ccccc4)ccc3-c3ccc4sc5c(ccc6c5c5ccccc5n6-c5ccccc5)c4c3)n2)cc1 ; c1ccc(-c2nc(-c3ccccc3)nc(-c3cc(-c4ccccc4-c4ccccc4)ccc3-c3cccc4sc5c(ccc6c5c5ccccc5n6-c5ccccc5)c34)n2)cc1 ; c1ccc(-c2ncnc(-c3cc(-c4ccccc4-c4ccccc4)ccc3-c3cccc4c3sc3cc5c6ccccc6n(-c6ccccc6)c5cc34)n2)cc1]

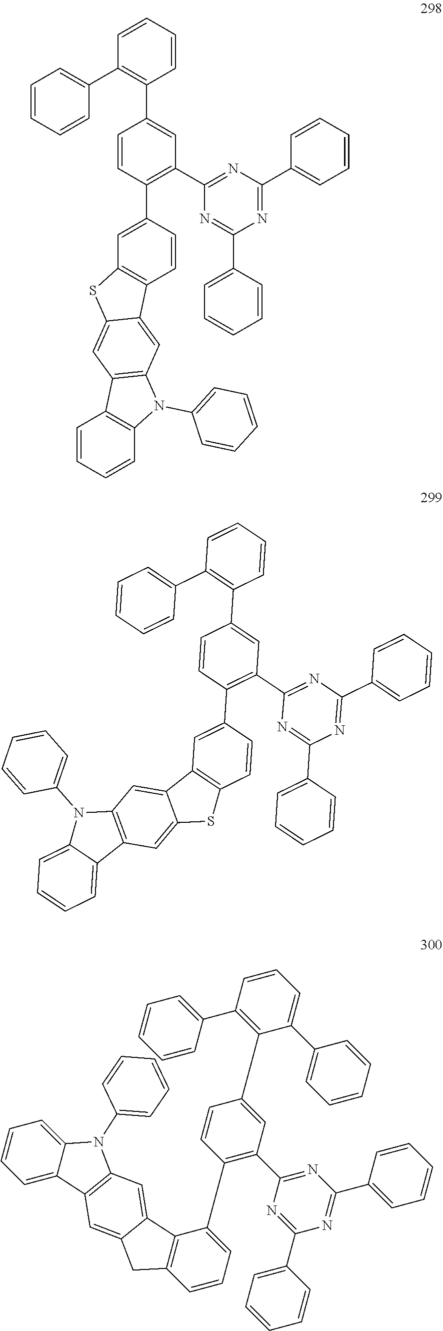 [SMILES: c1ccc(-c2nc(-c3ccccc3)nc(-c3cc(-c4ccccc4-c4ccccc4)ccc3-c3ccc4c(c3)sc3cc5c6ccccc6n(-c6ccccc6)c5cc34)n2)cc1 ; c1ccc(-c2nc(-c3ccccc3)nc(-c3cc(-c4ccccc4-c4ccccc4)ccc3-c3ccc4sc5cc6c7ccccc7n(-c7ccccc7)c6cc5c4c3)n2)cc1 ; c1ccc(-c2nc(-c3ccccc3)nc(-c3cc(Cc4c(-c5ccccc5)cccc4-c4ccccc4)ccc3Cc3cccc4c3-c3cc5c(cc3C4)c3ccccc3n5-c3ccccc3)n2)cc1]

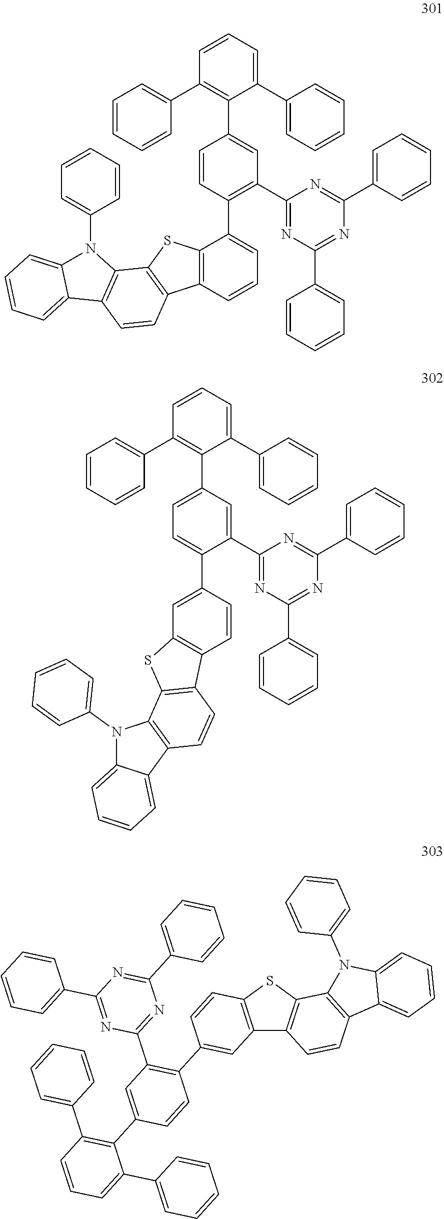 [SMILES: c1ccc(-c2nc(-c3ccccc3)nc(-c3cc(-c4c(-c5ccccc5)cccc4-c4ccccc4)ccc3-c3cccc4c3sc3c4ccc4c5ccccc5n(-c5ccccc5)c43)n2)cc1 ; c1ccc(-c2nc(-c3ccccc3)nc(-c3cc(-c4c(-c5ccccc5)cccc4-c4ccccc4)ccc3-c3ccc4c(c3)sc3c4ccc4c5ccccc5n(-c5ccccc5)c43)n2)cc1 ; c1ccc(-c2nc(-c3ccccc3)nc(-c3cc(-c4c(-c5ccccc5)cccc4-c4ccccc4)ccc3-c3ccc4sc5c(ccc6c7ccccc7n(-c7ccccc7)c65)c4c3)n2)cc1]

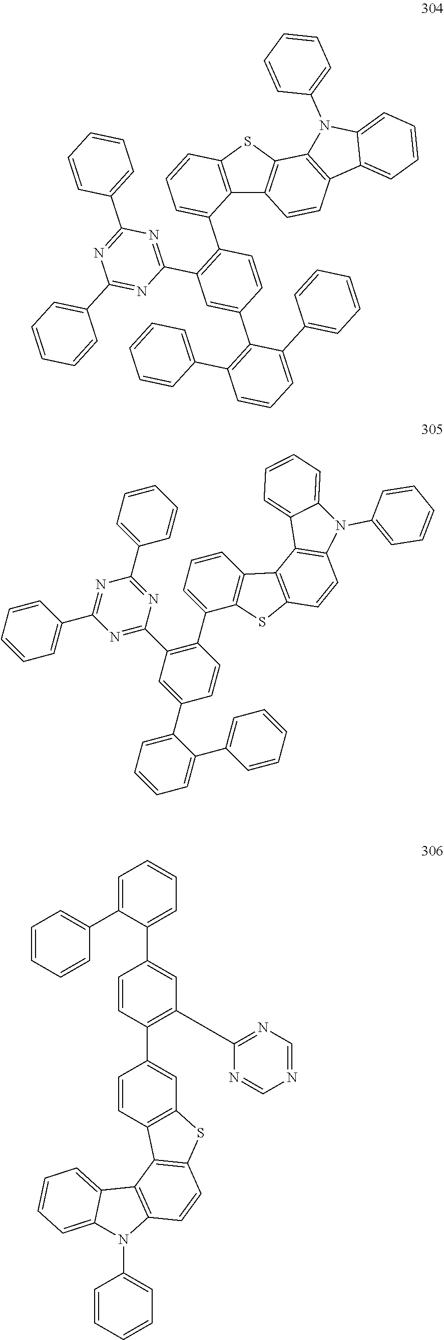 [SMILES: c1ccc(-c2nc(-c3ccccc3)nc(-c3cc(-c4c(-c5ccccc5)cccc4-c4ccccc4)ccc3-c3cccc4sc5c(ccc6c7ccccc7n(-c7ccccc7)c65)c34)n2)cc1 ; c1ccc(-c2nc(-c3ccccc3)nc(-c3cc(-c4ccccc4-c4ccccc4)ccc3-c3cccc4c3sc3ccc5c(c6ccccc6n5-c5ccccc5)c34)n2)cc1 ; c1ccc(-c2ccccc2-c2ccc(-c3ccc4c(c3)sc3ccc5c(c6ccccc6n5-c5ccccc5)c34)c(Cc3ncncn3)c2)cc1]

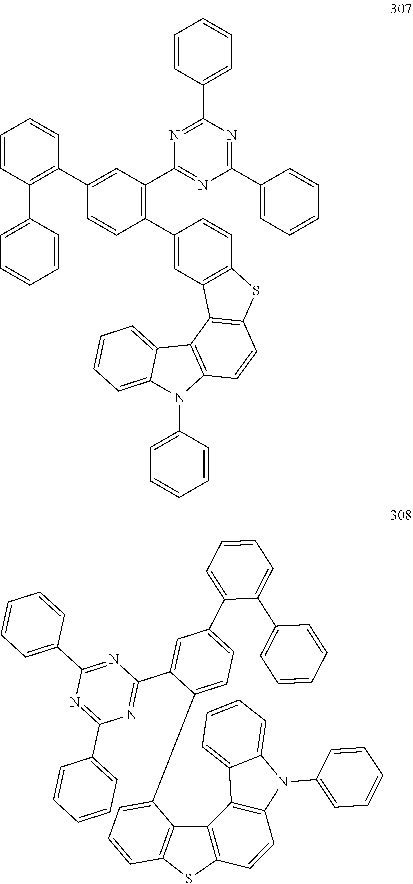 [SMILES: c1ccc(-c2nc(-c3ccccc3)nc(-c3cc(-c4ccccc4-c4ccccc4)ccc3-c3ccc4sc5ccc6c(c7ccccc7n6-c6ccccc6)c5c4c3)n2)cc1 ; c1ccc(-c2nc(-c3ccccc3)nc(-c3cc(-c4ccccc4-c4ccccc4)ccc3-c3cccc4sc5ccc6c(c7ccccc7n6-c6ccccc6)c5c34)n2)cc1]

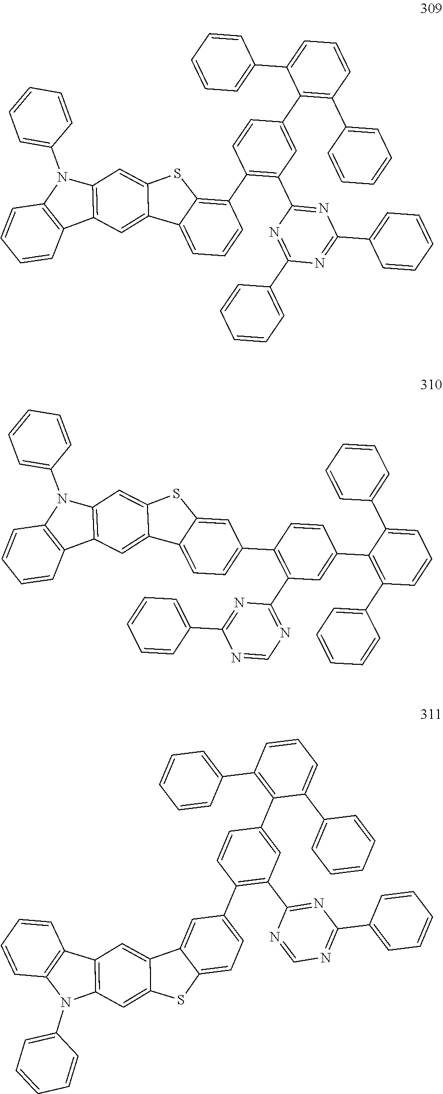 [SMILES: c1ccc(-c2nc(-c3ccccc3)nc(-c3cc(-c4c(-c5ccccc5)cccc4-c4ccccc4)ccc3-c3cccc4c3sc3cc5c(cc34)c3ccccc3n5-c3ccccc3)n2)cc1 ; c1ccc(-c2ncnc(-c3cc(-c4c(-c5ccccc5)cccc4-c4ccccc4)ccc3-c3ccc4c(c3)sc3cc5c(cc34)c3ccccc3n5-c3ccccc3)n2)cc1 ; c1ccc(-c2ncnc(-c3cc(-c4c(-c5ccccc5)cccc4-c4ccccc4)ccc3-c3ccc4sc5cc6c(cc5c4c3)c3ccccc3n6-c3ccccc3)n2)cc1]

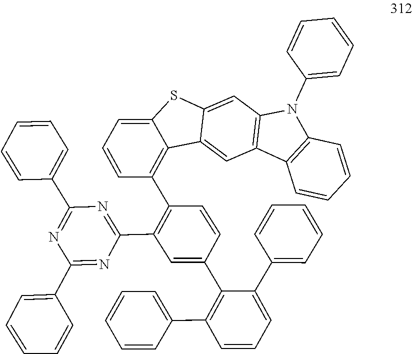 [SMILES: c1ccc(-c2nc(-c3ccccc3)nc(-c3cc(-c4c(-c5ccccc5)cccc4-c4ccccc4)ccc3-c3cccc4sc5cc6c(cc5c34)c3ccccc3n6-c3ccccc3)n2)cc1]

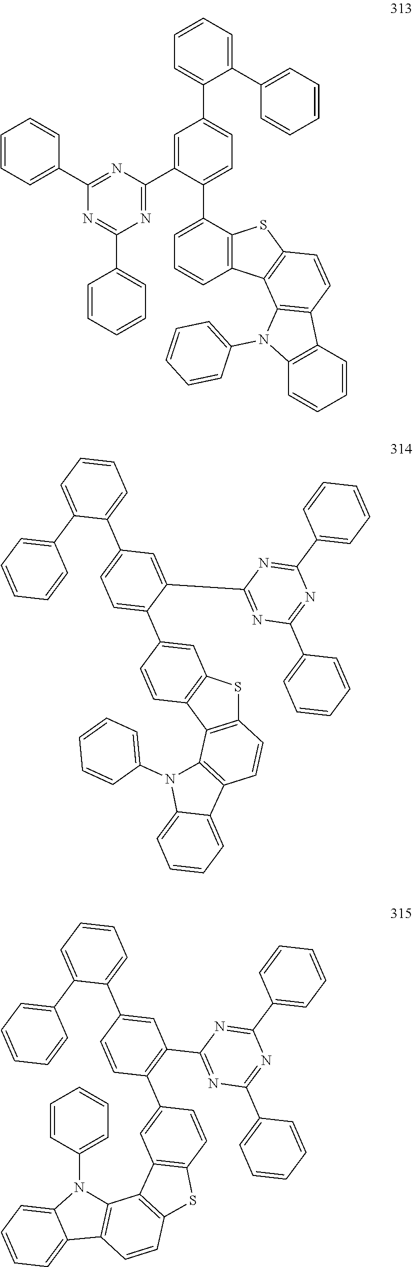 [SMILES: c1ccc(-c2nc(-c3ccccc3)nc(-c3cc(-c4ccccc4-c4ccccc4)ccc3-c3cccc4c3sc3ccc5c6ccccc6n(-c6ccccc6)c5c34)n2)cc1 ; c1ccc(-c2nc(-c3ccccc3)nc(-c3cc(-c4ccccc4-c4ccccc4)ccc3-c3ccc4c(c3)sc3ccc5c6ccccc6n(-c6ccccc6)c5c34)n2)cc1 ; c1ccc(-c2nc(-c3ccccc3)nc(-c3cc(-c4ccccc4-c4ccccc4)ccc3-c3ccc4sc5ccc6c7ccccc7n(-c7ccccc7)c6c5c4c3)n2)cc1]

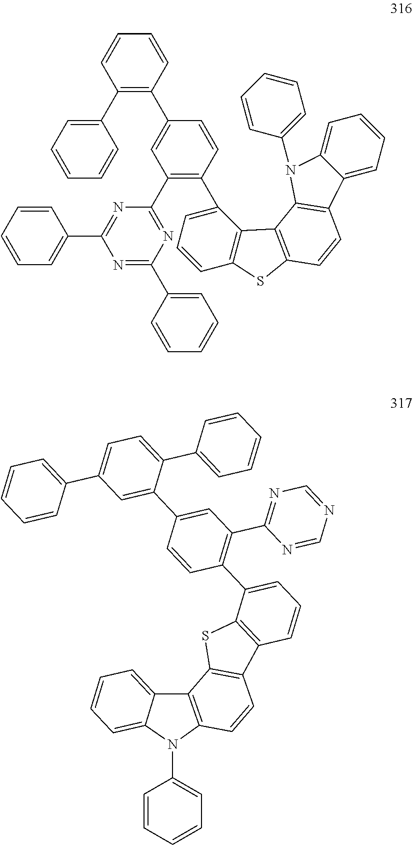 [SMILES: c1ccc(-c2nc(-c3ccccc3)nc(-c3cc(-c4ccccc4-c4ccccc4)ccc3-c3cccc4sc5ccc6c7ccccc7n(-c7ccccc7)c6c5c34)n2)cc1 ; c1ccc(-c2ccc(-c3ccccc3)c(-c3ccc(-c4cccc5c4sc4c5ccc5c4c4ccccc4n5-c4ccccc4)c(-c4ncncn4)c3)c2)cc1]

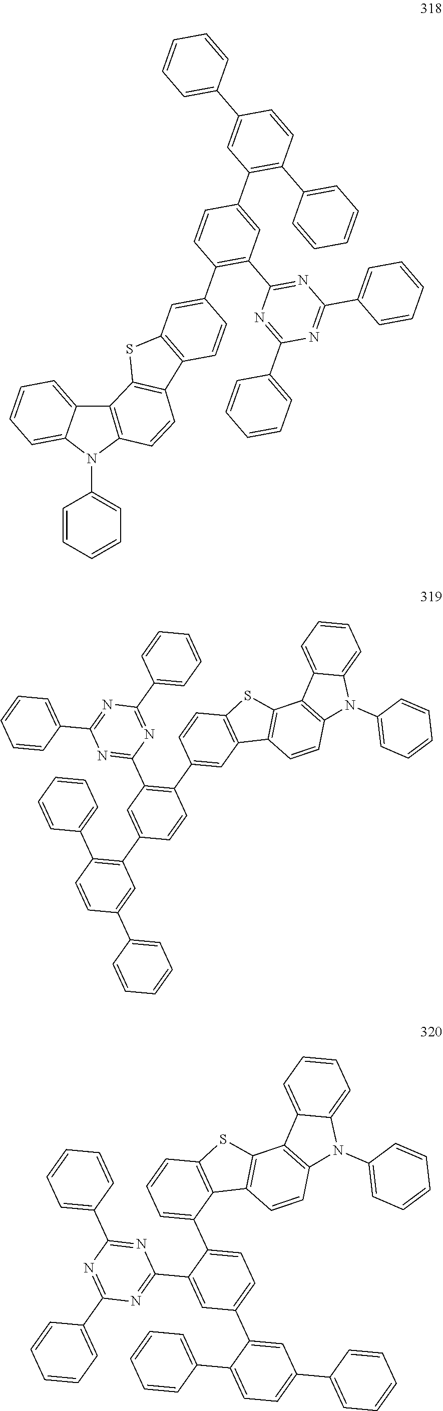 [SMILES: c1ccc(-c2ccc(-c3ccccc3)c(-c3ccc(-c4ccc5c(c4)sc4c5ccc5c4c4ccccc4n5-c4ccccc4)c(-c4nc(-c5ccccc5)nc(-c5ccccc5)n4)c3)c2)cc1 ; c1ccc(-c2ccc(-c3ccccc3)c(-c3ccc(-c4ccc5sc6c(ccc7c6c6ccccc6n7-c6ccccc6)c5c4)c(-c4nc(-c5ccccc5)nc(-c5ccccc5)n4)c3)c2)cc1 ; c1ccc(-c2ccc(-c3ccccc3)c(-c3ccc(-c4cccc5sc6c(ccc7c6c6ccccc6n7-c6ccccc6)c45)c(-c4nc(-c5ccccc5)nc(-c5ccccc5)n4)c3)c2)cc1]

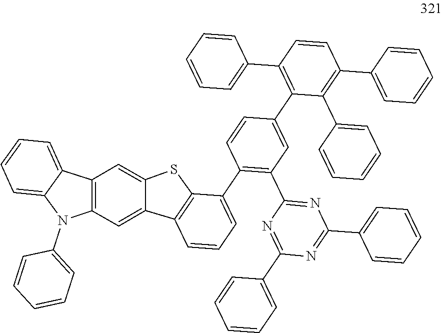 [SMILES: c1ccc(-c2nc(-c3ccccc3)nc(-c3cc(-c4c(-c5ccccc5)ccc(-c5ccccc5)c4-c4ccccc4)ccc3-c3cccc4c3sc3cc5c6ccccc6n(-c6ccccc6)c5cc34)n2)cc1]

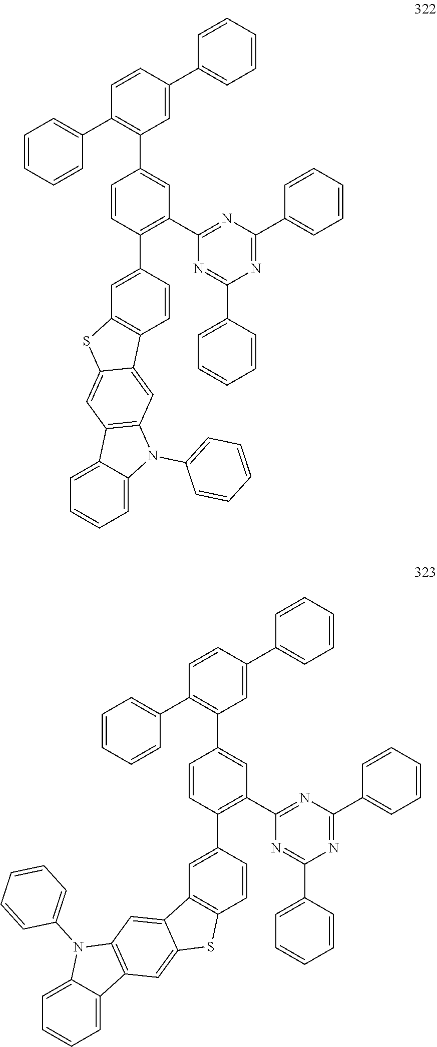 [SMILES: c1ccc(-c2ccc(-c3ccccc3)c(-c3ccc(-c4ccc5c(c4)sc4cc6c7ccccc7n(-c7ccccc7)c6cc45)c(-c4nc(-c5ccccc5)nc(-c5ccccc5)n4)c3)c2)cc1 ; c1ccc(-c2ccc(-c3ccccc3)c(-c3ccc(-c4ccc5sc6cc7c8ccccc8n(-c8ccccc8)c7cc6c5c4)c(-c4nc(-c5ccccc5)nc(-c5ccccc5)n4)c3)c2)cc1]

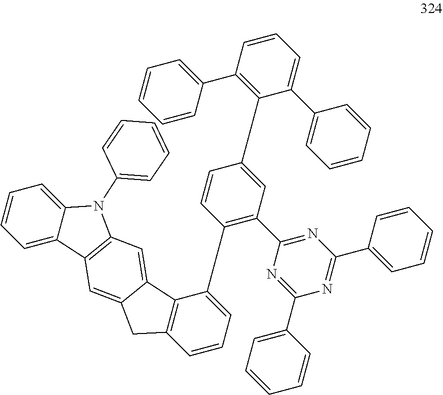 [SMILES: c1ccc(-c2nc(-c3ccccc3)nc(-c3cc(Cc4c(-c5ccccc5)cccc4-c4ccccc4)ccc3Cc3cccc4c3-c3cc5c(cc3C4)c3ccccc3n5-c3ccccc3)n2)cc1]

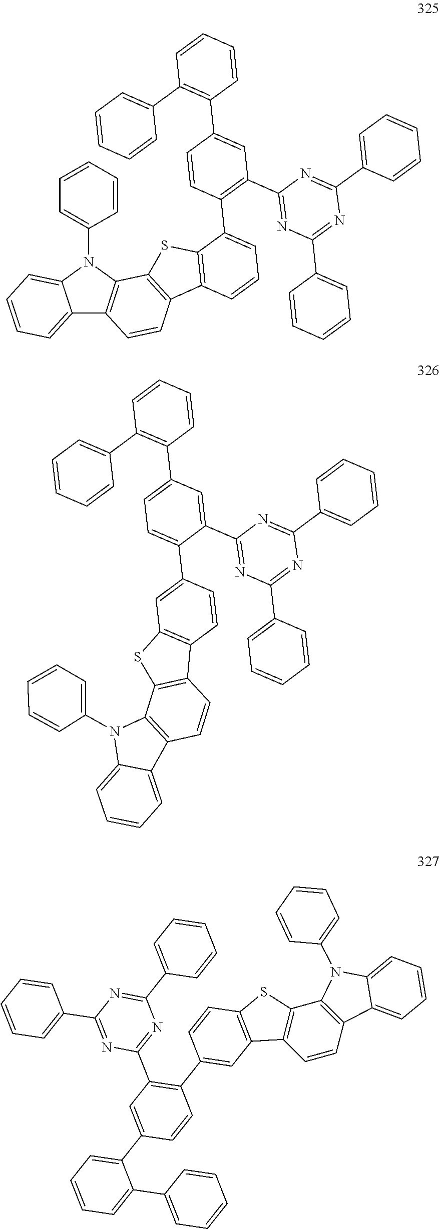 [SMILES: c1ccc(-c2nc(-c3ccccc3)nc(-c3cc(-c4ccccc4-c4ccccc4)ccc3-c3cccc4c3sc3c4ccc4c5ccccc5n(-c5ccccc5)c43)n2)cc1 ; c1ccc(-c2nc(-c3ccccc3)nc(-c3cc(-c4ccccc4-c4ccccc4)ccc3-c3ccc4c(c3)sc3c4ccc4c5ccccc5n(-c5ccccc5)c43)n2)cc1 ; c1ccc(-c2nc(-c3ccccc3)nc(-c3cc(-c4ccccc4-c4ccccc4)ccc3-c3ccc4sc5c(ccc6c7ccccc7n(-c7ccccc7)c65)c4c3)n2)cc1]

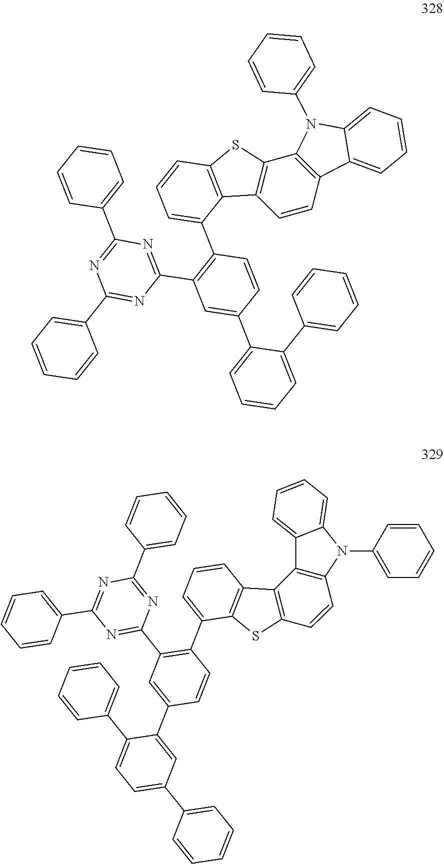 [SMILES: c1ccc(-c2nc(-c3ccccc3)nc(-c3cc(-c4ccccc4-c4ccccc4)ccc3-c3cccc4sc5c(ccc6c7ccccc7n(-c7ccccc7)c65)c34)n2)cc1 ; c1ccc(-c2ccc(-c3ccccc3)c(-c3ccc(-c4cccc5c4sc4ccc6c(c7ccccc7n6-c6ccccc6)c45)c(-c4nc(-c5ccccc5)nc(-c5ccccc5)n4)c3)c2)cc1]

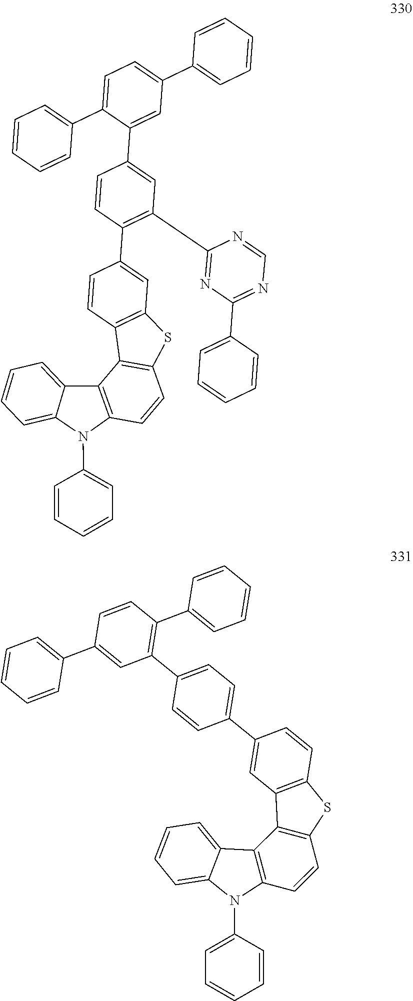 [SMILES: c1ccc(-c2ccc(-c3ccccc3)c(-c3ccc(-c4ccc5c(c4)sc4ccc6c(c7ccccc7n6-c6ccccc6)c45)c(Cc4ncnc(-c5ccccc5)n4)c3)c2)cc1 ; c1ccc(-c2ccc(-c3ccccc3)c(-c3ccc(-c4ccc5sc6ccc7c(c8ccccc8n7-c7ccccc7)c6c5c4)cc3)c2)cc1]

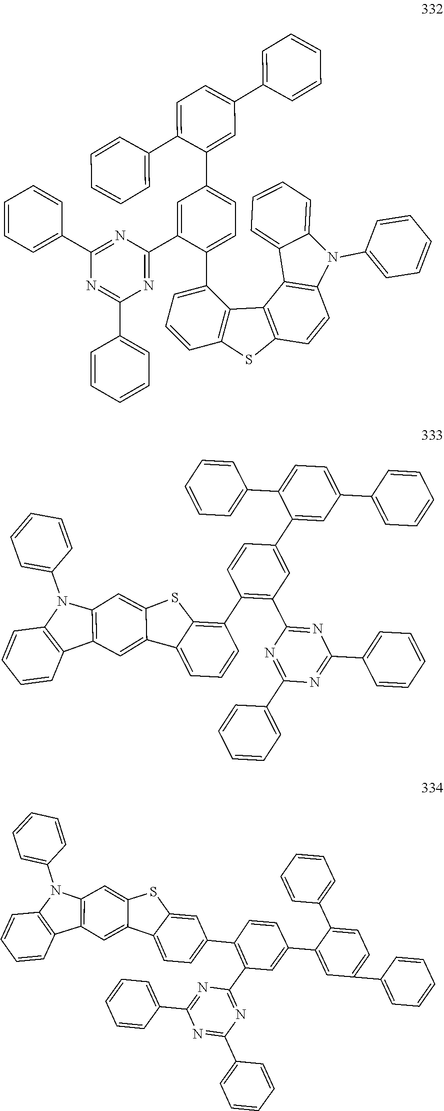 [SMILES: c1ccc(-c2ccc(-c3ccccc3)c(-c3ccc(-c4cccc5sc6ccc7c(c8ccccc8n7-c7ccccc7)c6c45)c(-c4nc(-c5ccccc5)nc(-c5ccccc5)n4)c3)c2)cc1 ; c1ccc(-c2ccc(-c3ccccc3)c(-c3ccc(-c4cccc5c4sc4cc6c(cc45)c4ccccc4n6-c4ccccc4)c(-c4nc(-c5ccccc5)nc(-c5ccccc5)n4)c3)c2)cc1 ; c1ccc(-c2ccc(-c3ccccc3)c(-c3ccc(-c4ccc5c(c4)sc4cc6c(cc45)c4ccccc4n6-c4ccccc4)c(-c4nc(-c5ccccc5)nc(-c5ccccc5)n4)c3)c2)cc1]

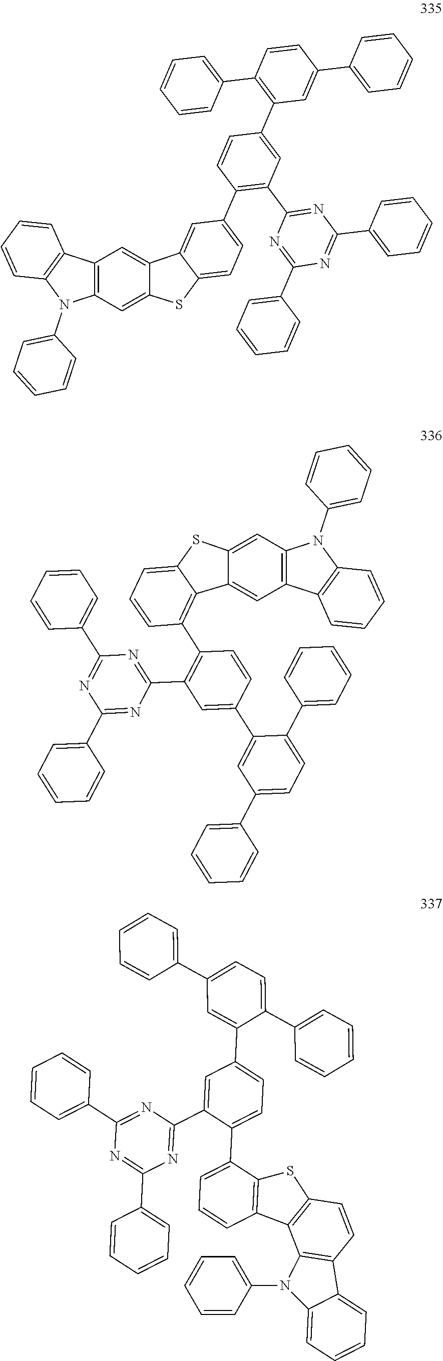 [SMILES: c1ccc(-c2ccc(-c3ccccc3)c(-c3ccc(-c4ccc5sc6cc7c(cc6c5c4)c4ccccc4n7-c4ccccc4)c(-c4nc(-c5ccccc5)nc(-c5ccccc5)n4)c3)c2)cc1 ; c1ccc(-c2ccc(-c3ccccc3)c(-c3ccc(-c4cccc5sc6cc7c(cc6c45)c4ccccc4n7-c4ccccc4)c(-c4nc(-c5ccccc5)nc(-c5ccccc5)n4)c3)c2)cc1 ; c1ccc(-c2ccc(-c3ccccc3)c(-c3ccc(-c4cccc5c4sc4ccc6c7ccccc7n(-c7ccccc7)c6c45)c(-c4nc(-c5ccccc5)nc(-c5ccccc5)n4)c3)c2)cc1]

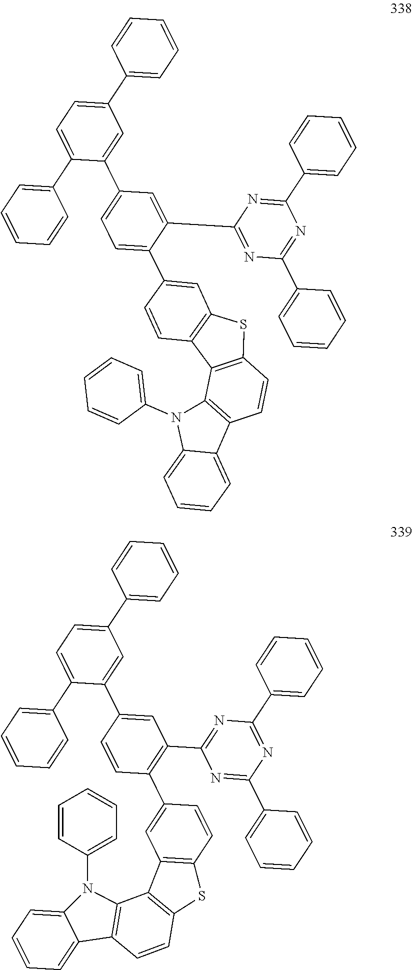

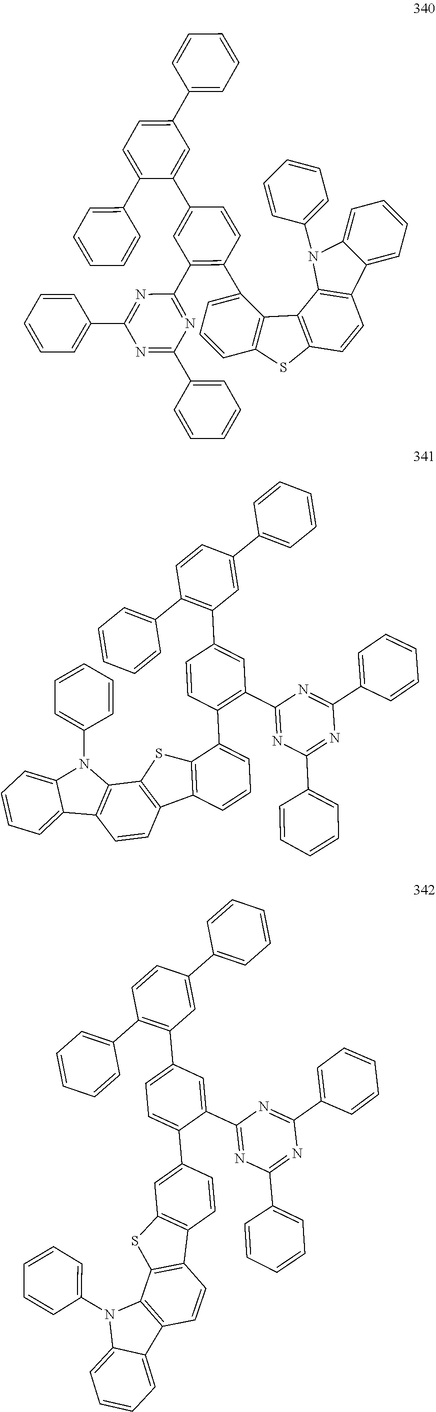 [SMILES: c1ccc(-c2ccc(-c3ccccc3)c(-c3ccc(-c4cccc5sc6ccc7c8ccccc8n(-c8ccccc8)c7c6c45)c(-c4nc(-c5ccccc5)nc(-c5ccccc5)n4)c3)c2)cc1 ; c1ccc(-c2ccc(-c3ccccc3)c(-c3ccc(-c4cccc5c4sc4c5ccc5c6ccccc6n(-c6ccccc6)c54)c(-c4nc(-c5ccccc5)nc(-c5ccccc5)n4)c3)c2)cc1 ; c1ccc(-c2ccc(-c3ccccc3)c(-c3ccc(-c4ccc5c(c4)sc4c5ccc5c6ccccc6n(-c6ccccc6)c54)c(-c4nc(-c5ccccc5)nc(-c5ccccc5)n4)c3)c2)cc1]

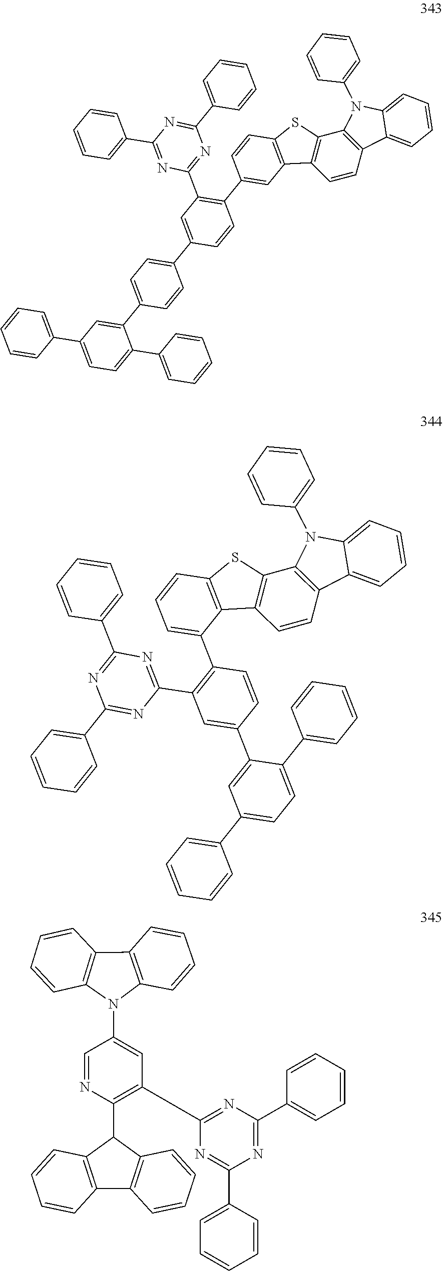

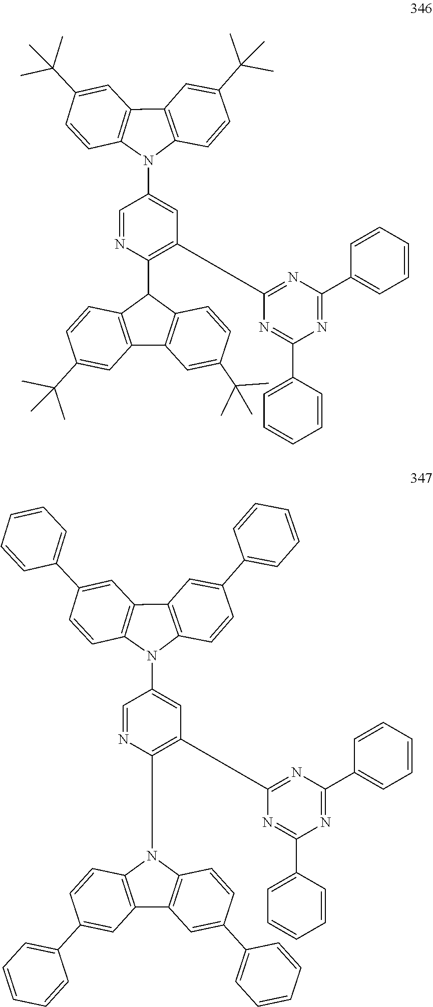 [SMILES: CC(C)(C)c1ccc2c(c1)-c1cc(C(C)(C)C)ccc1C2c1ncc(-n2c3ccc(C(C)(C)C)cc3c3cc(C(C)(C)C)ccc32)cc1CCc1nc(-c2ccccc2)nc(-c2ccccc2)n1 ; c1ccc(-c2ccc3c(c2)c2cc(-c4ccccc4)ccc2n3CCc2ncc(-n3c4ccc(-c5ccccc5)cc4c4cc(-c5ccccc5)ccc43)cc2CCc2nc(-c3ccccc3)nc(-c3ccccc3)n2)cc1]

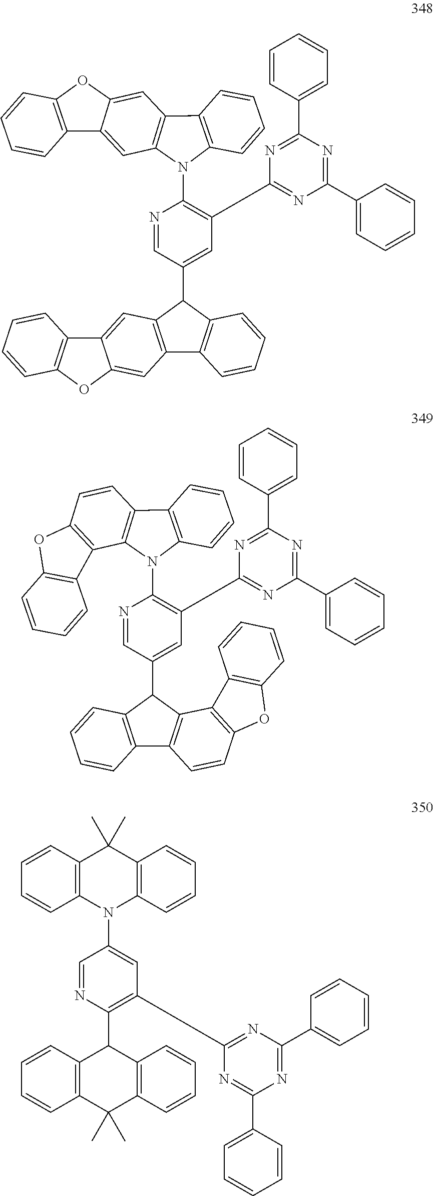 [SMILES: c1ccc(-c2nc(Cc3cc(C4c5ccccc5-c5cc6oc7ccccc7c6cc54)cnc3-n3c4ccccc4c4cc5oc6ccccc6c5cc43)nc(-c3ccccc3)n2)cc1 ; c1ccc(-c2nc(Cc3cc(C4c5ccccc5-c5ccc6oc7ccccc7c6c54)cnc3-n3c4ccccc4c4ccc5oc6ccccc6c5c43)nc(-c3ccccc3)n2)cc1 ; CC1(C)c2ccccc2C(c2ncc(N3c4ccccc4C(C)(C)c4ccccc43)cc2CCc2nc(-c3ccccc3)nc(-c3ccccc3)n2)c2ccccc21]

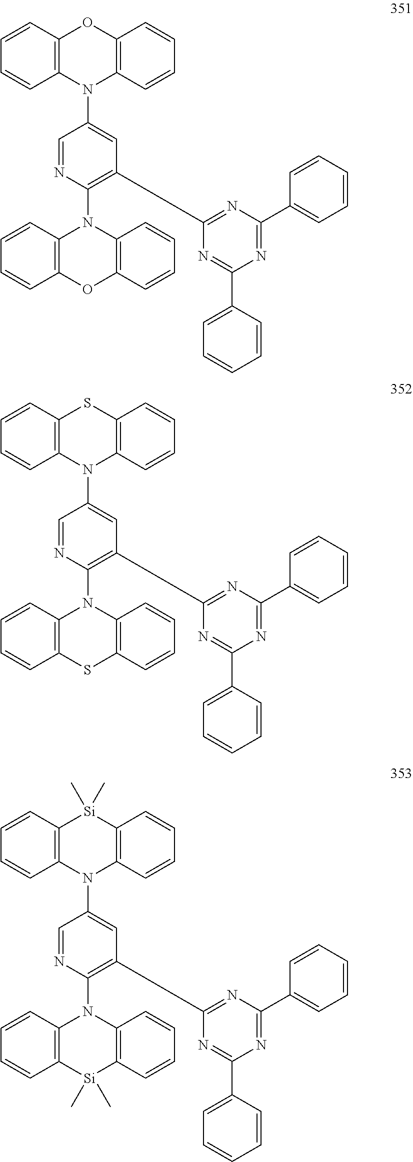 [SMILES: c1ccc(-c2nc(CCc3cc(N4c5ccccc5Oc5ccccc54)cnc3N3c4ccccc4Oc4ccccc43)nc(-c3ccccc3)n2)cc1 ; c1ccc(-c2nc(CCc3cc(N4c5ccccc5Sc5ccccc54)cnc3N3c4ccccc4Sc4ccccc43)nc(-c3ccccc3)n2)cc1 ; C[Si]1(C)c2ccccc2N(c2cnc(N3c4ccccc4[Si](C)(C)c4ccccc43)c(CCc3nc(-c4ccccc4)nc(-c4ccccc4)n3)c2)c2ccccc21]

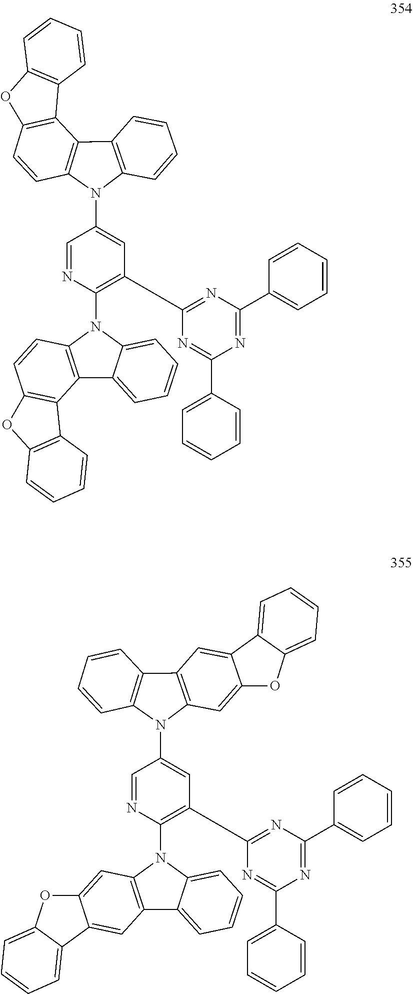 [SMILES: c1ccc(-c2nc(Cc3cc(-n4c5ccccc5c5c6c(ccc54)oc4ccccc46)cnc3-n3c4ccccc4c4c5c(ccc43)oc3ccccc35)nc(-c3ccccc3)n2)cc1 ; c1ccc(-c2nc(Cc3cc(-n4c5ccccc5c5cc6c(cc54)oc4ccccc46)cnc3-n3c4ccccc4c4cc5c(cc43)oc3ccccc35)nc(-c3ccccc3)n2)cc1]

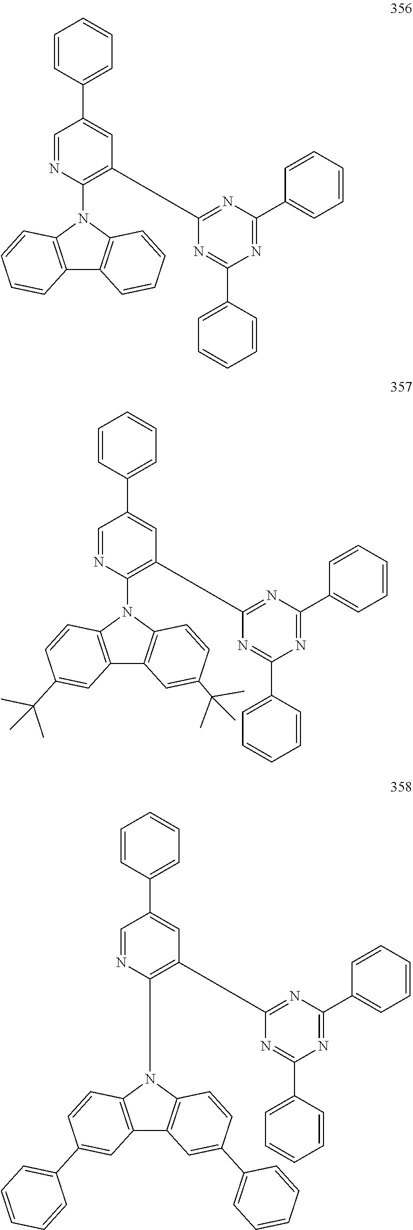 [SMILES: c1ccc(-c2cnc(-n3c4ccccc4c4ccccc43)c(CCc3nc(-c4ccccc4)nc(-c4ccccc4)n3)c2)cc1 ; CC(C)(C)c1ccc2c(c1)c1cc(C(C)(C)C)ccc1n2-c1ncc(-c2ccccc2)cc1CCc1nc(-c2ccccc2)nc(-c2ccccc2)n1 ; c1ccc(-c2cnc(CCn3c4ccc(-c5ccccc5)cc4c4cc(-c5ccccc5)ccc43)c(CCc3nc(-c4ccccc4)nc(-c4ccccc4)n3)c2)cc1]

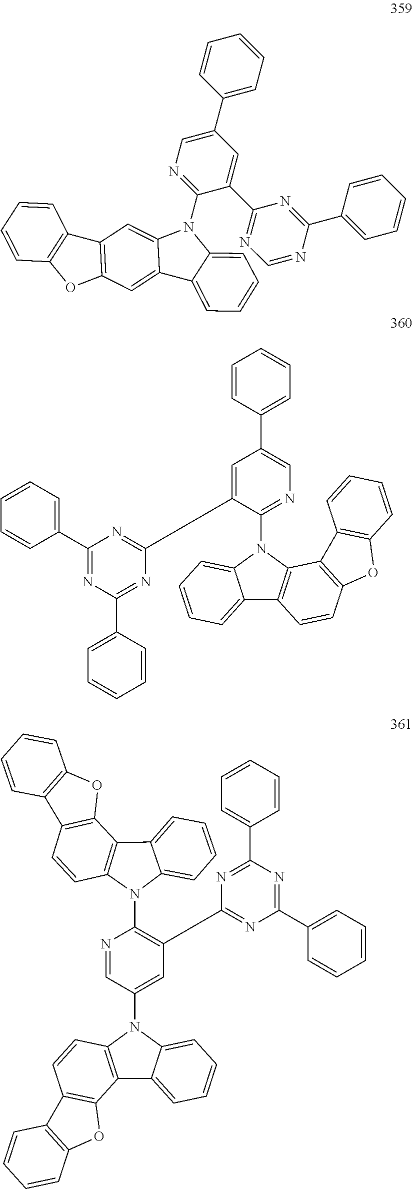

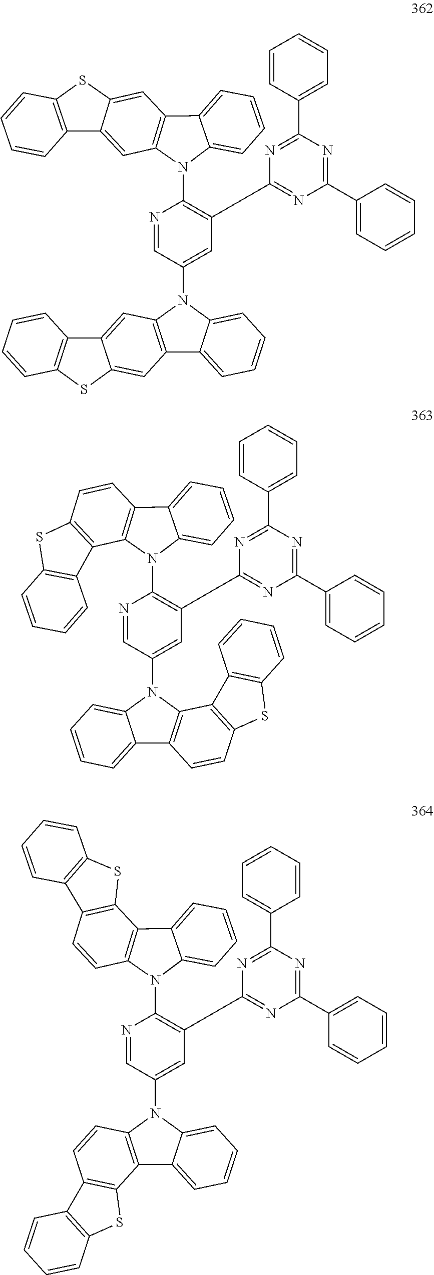 [SMILES: c1ccc(-c2nc(Cc3cc(-n4c5ccccc5c5cc6sc7ccccc7c6cc54)cnc3-n3c4ccccc4c4cc5sc6ccccc6c5cc43)nc(-c3ccccc3)n2)cc1 ; c1ccc(-c2nc(Cc3cc(-n4c5ccccc5c5ccc6sc7ccccc7c6c54)cnc3-n3c4ccccc4c4ccc5sc6ccccc6c5c43)nc(-c3ccccc3)n2)cc1 ; c1ccc(-c2nc(Cc3cc(-n4c5ccccc5c5c6sc7ccccc7c6ccc54)cnc3-n3c4ccccc4c4c5sc6ccccc6c5ccc43)nc(-c3ccccc3)n2)cc1]

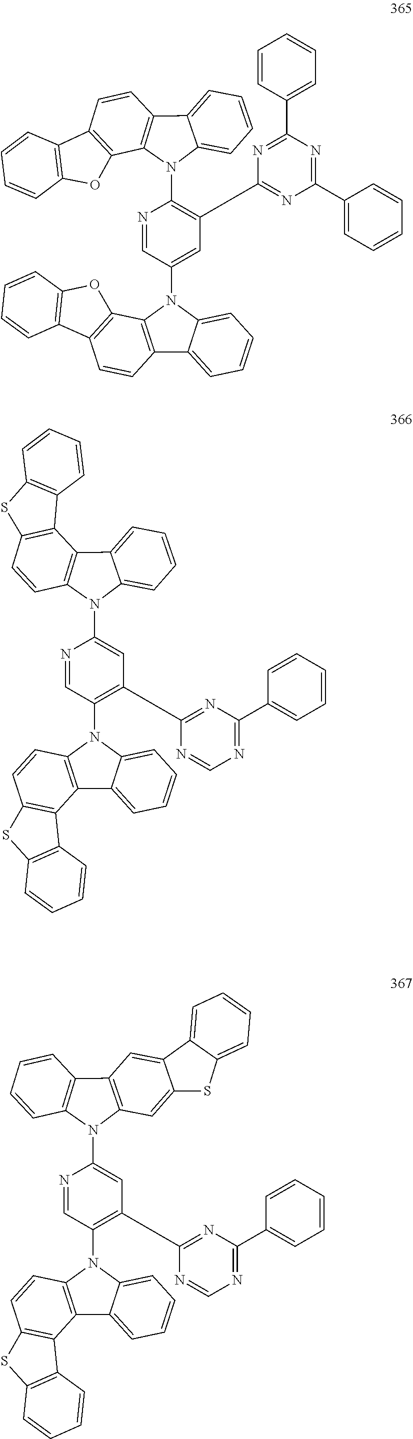 [SMILES: c1ccc(-c2nc(Cc3cc(-n4c5ccccc5c5ccc6c7ccccc7oc6c54)cnc3-n3c4ccccc4c4ccc5c6ccccc6oc5c43)nc(-c3ccccc3)n2)cc1 ; c1ccc(-c2ncnc(Cc3cc(-n4c5ccccc5c5c6c(ccc54)sc4ccccc46)ncc3-n3c4ccccc4c4c5c(ccc43)sc3ccccc35)n2)cc1 ; c1ccc(-c2ncnc(Cc3cc(-n4c5ccccc5c5cc6c(cc54)sc4ccccc46)ncc3-n3c4ccccc4c4c5c(ccc43)sc3ccccc35)n2)cc1]

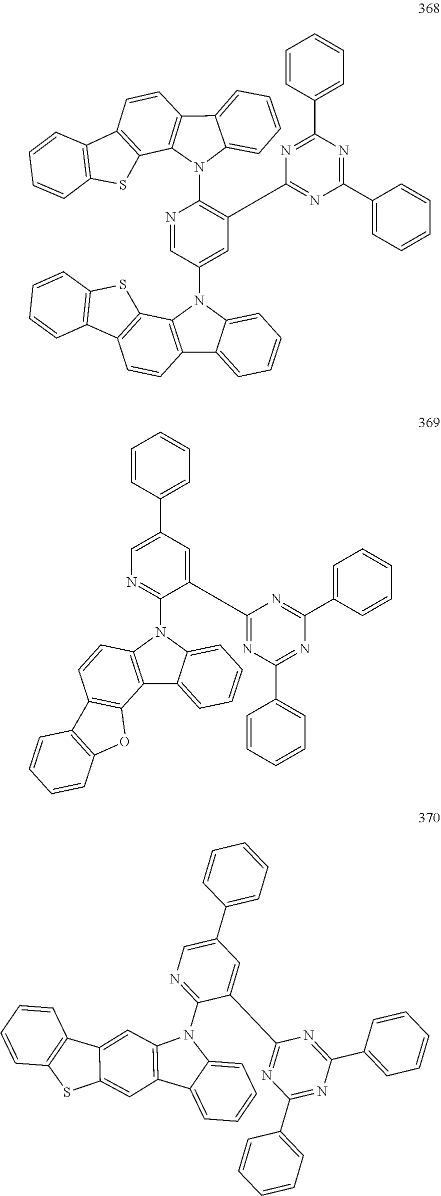 [SMILES: c1ccc(-c2nc(Cc3cc(-n4c5ccccc5c5ccc6c7ccccc7sc6c54)cnc3-n3c4ccccc4c4ccc5c6ccccc6sc5c43)nc(-c3ccccc3)n2)cc1 ; c1ccc(-c2cnc(-n3c4ccccc4c4c5oc6ccccc6c5ccc43)c(Cc3nc(-c4ccccc4)nc(-c4ccccc4)n3)c2)cc1 ; c1ccc(-c2cnc(-n3c4ccccc4c4cc5sc6ccccc6c5cc43)c(Cc3nc(-c4ccccc4)nc(-c4ccccc4)n3)c2)cc1]

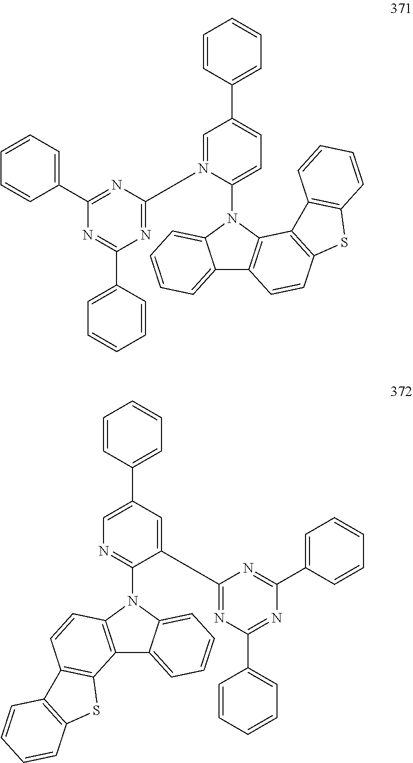

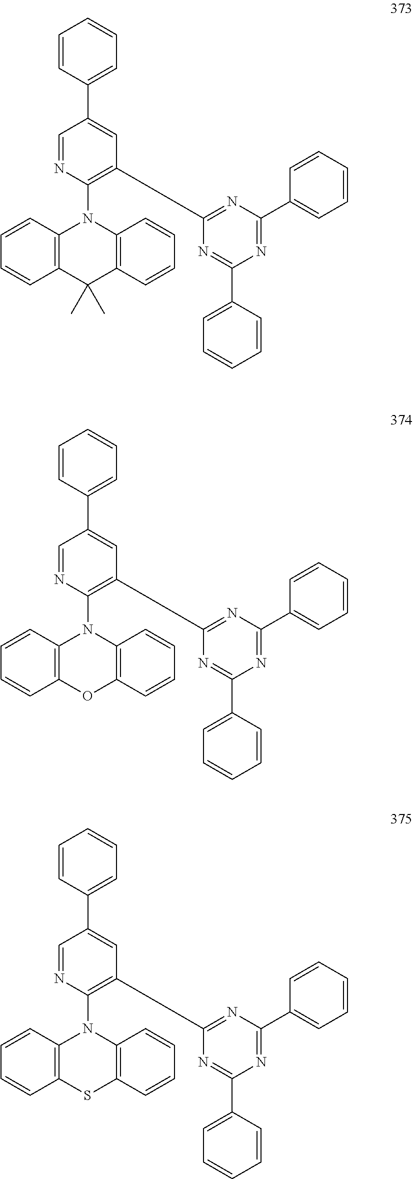 [SMILES: CC1(C)c2ccccc2N(c2ncc(-c3ccccc3)cc2CCc2nc(-c3ccccc3)nc(-c3ccccc3)n2)c2ccccc21 ; c1ccc(-c2cnc(N3c4ccccc4Oc4ccccc43)c(CCc3nc(-c4ccccc4)nc(-c4ccccc4)n3)c2)cc1 ; c1ccc(-c2cnc(N3c4ccccc4Sc4ccccc43)c(CCc3nc(-c4ccccc4)nc(-c4ccccc4)n3)c2)cc1]

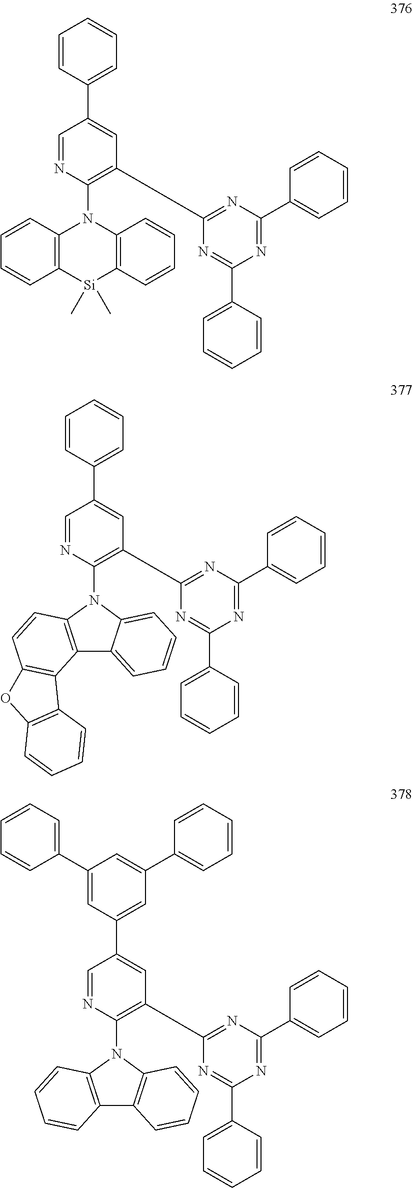

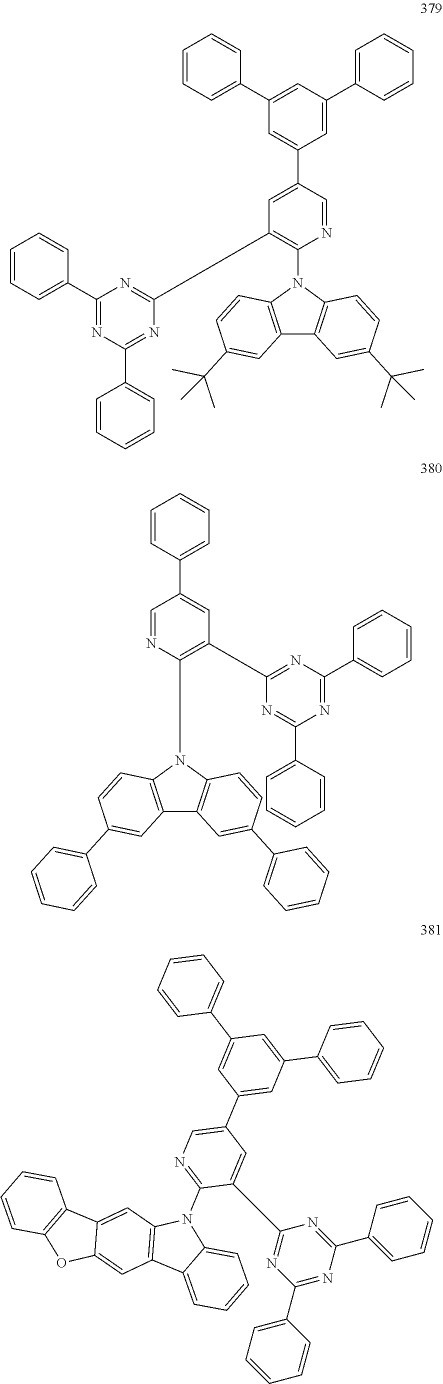 [SMILES: CC(C)(C)c1ccc2c(c1)c1cc(C(C)(C)C)ccc1n2-c1ncc(-c2cc(-c3ccccc3)cc(-c3ccccc3)c2)cc1CCCc1nc(-c2ccccc2)nc(-c2ccccc2)n1 ; c1ccc(-c2cnc(CCn3c4ccc(-c5ccccc5)cc4c4cc(-c5ccccc5)ccc43)c(Cc3nc(-c4ccccc4)nc(-c4ccccc4)n3)c2)cc1 ; c1ccc(-c2cc(-c3ccccc3)cc(-c3cnc(-n4c5ccccc5c5cc6oc7ccccc7c6cc54)c(Cc4nc(-c5ccccc5)nc(-c5ccccc5)n4)c3)c2)cc1]

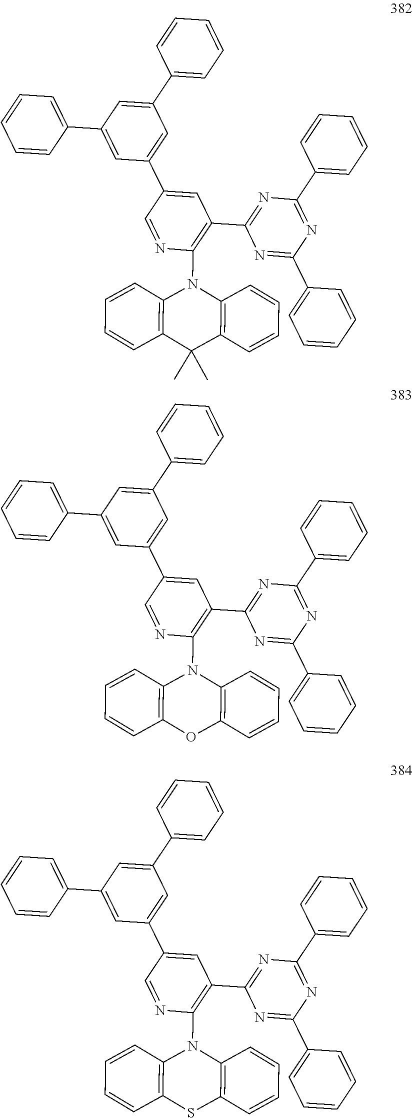

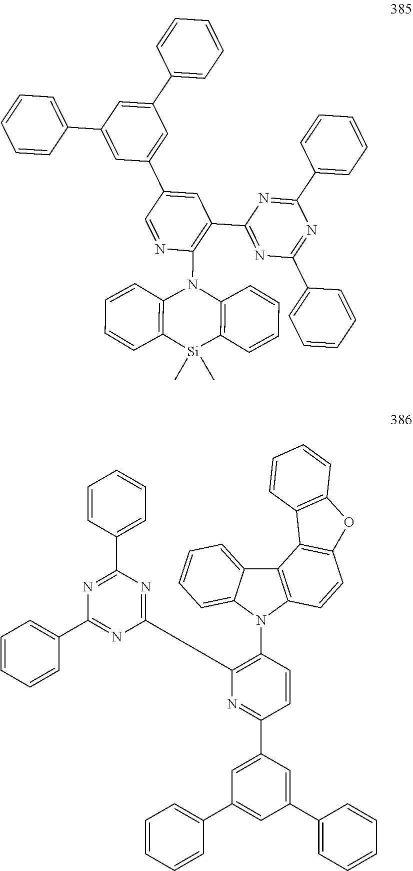 [SMILES: C[Si]1(C)c2ccccc2N(c2ncc(-c3cc(-c4ccccc4)cc(-c4ccccc4)c3)cc2-c2nc(-c3ccccc3)nc(-c3ccccc3)n2)c2ccccc21 ; c1ccc(-c2cc(-c3ccccc3)cc(-c3ccc(-n4c5ccccc5c5c6c(ccc54)oc4ccccc46)c(CCc4nc(-c5ccccc5)nc(-c5ccccc5)n4)n3)c2)cc1]

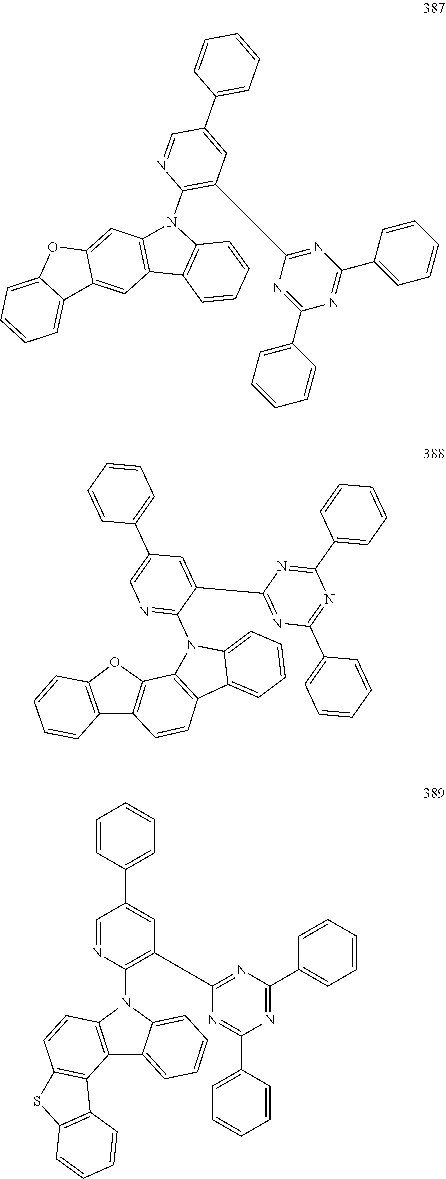 [SMILES: c1ccc(-c2cnc(-n3c4ccccc4c4cc5c(cc43)oc3ccccc35)c(CCc3nc(-c4ccccc4)nc(-c4ccccc4)n3)c2)cc1 ; c1ccc(-c2cnc(-n3c4ccccc4c4ccc5c6ccccc6oc5c43)c(Cc3nc(-c4ccccc4)nc(-c4ccccc4)n3)c2)cc1 ; c1ccc(-c2cnc(-n3c4ccccc4c4c5c(ccc43)sc3ccccc35)c(Cc3nc(-c4ccccc4)nc(-c4ccccc4)n3)c2)cc1]

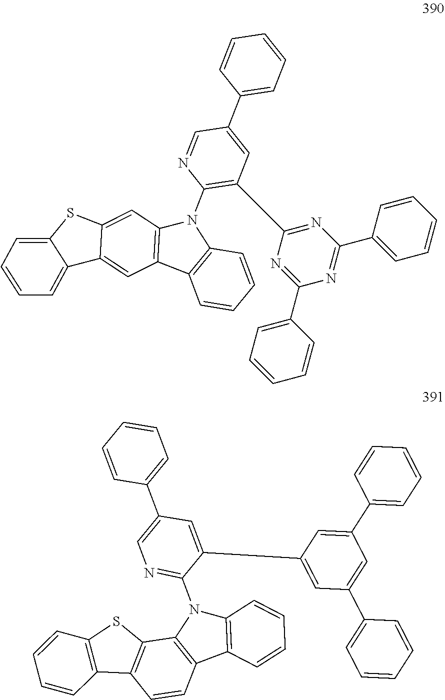 [SMILES: c1ccc(-c2cnc(-n3c4ccccc4c4cc5c(cc43)sc3ccccc35)c(Cc3nc(-c4ccccc4)nc(-c4ccccc4)n3)c2)cc1 ; c1ccc(-c2cc(-c3ccccc3)cc(-c3cc(-c4ccccc4)cnc3-n3c4ccccc4c4ccc5c6ccccc6sc5c43)c2)cc1]

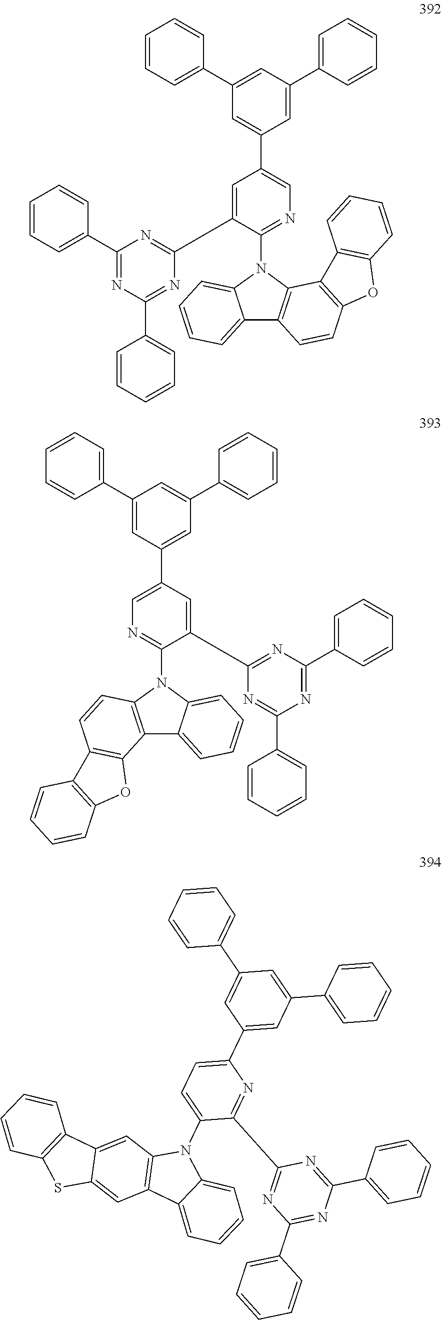 [SMILES: c1ccc(-c2cc(-c3ccccc3)cc(-c3cnc(-n4c5ccccc5c5ccc6oc7ccccc7c6c54)c(Cc4nc(-c5ccccc5)nc(-c5ccccc5)n4)c3)c2)cc1 ; c1ccc(-c2cc(-c3ccccc3)cc(-c3cnc(-n4c5ccccc5c5c6oc7ccccc7c6ccc54)c(Cc4nc(-c5ccccc5)nc(-c5ccccc5)n4)c3)c2)cc1 ; c1ccc(-c2cc(-c3ccccc3)cc(-c3ccc(-n4c5ccccc5c5cc6sc7ccccc7c6cc54)c(Cc4nc(-c5ccccc5)nc(-c5ccccc5)n4)n3)c2)cc1]

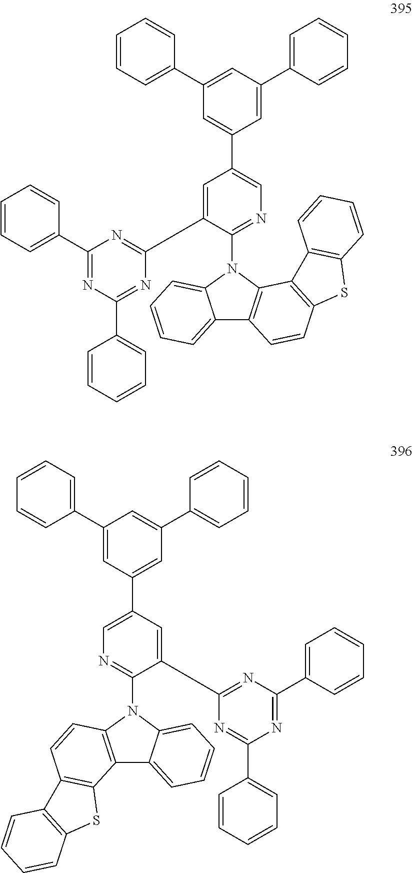 [SMILES: c1ccc(-c2cc(-c3ccccc3)cc(-c3cnc(-n4c5ccccc5c5ccc6sc7ccccc7c6c54)c(Cc4nc(-c5ccccc5)nc(-c5ccccc5)n4)c3)c2)cc1 ; c1ccc(-c2cc(-c3ccccc3)cc(-c3cnc(-n4c5ccccc5c5c6sc7ccccc7c6ccc54)c(Cc4nc(-c5ccccc5)nc(-c5ccccc5)n4)c3)c2)cc1]

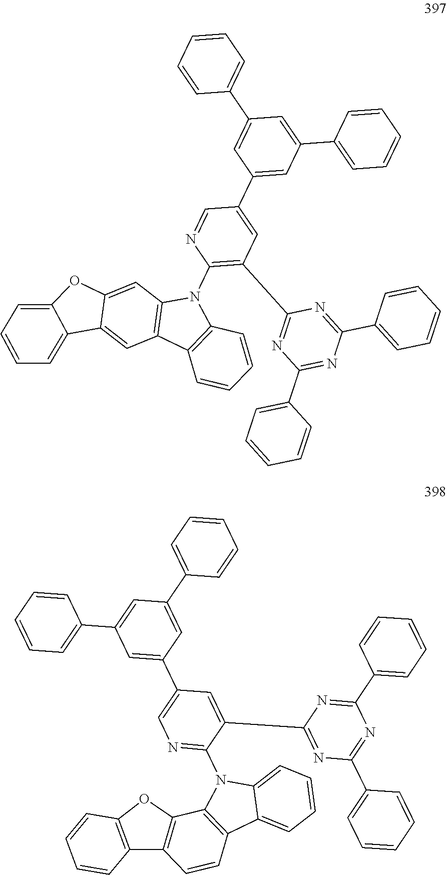 [SMILES: c1ccc(-c2cc(-c3ccccc3)cc(-c3cnc(-n4c5ccccc5c5cc6c(cc54)oc4ccccc46)c(Cc4nc(-c5ccccc5)nc(-c5ccccc5)n4)c3)c2)cc1 ; c1ccc(-c2cc(-c3ccccc3)cc(-c3cnc(-n4c5ccccc5c5ccc6c7ccccc7oc6c54)c(-c4nc(-c5ccccc5)nc(-c5ccccc5)n4)c3)c2)cc1]

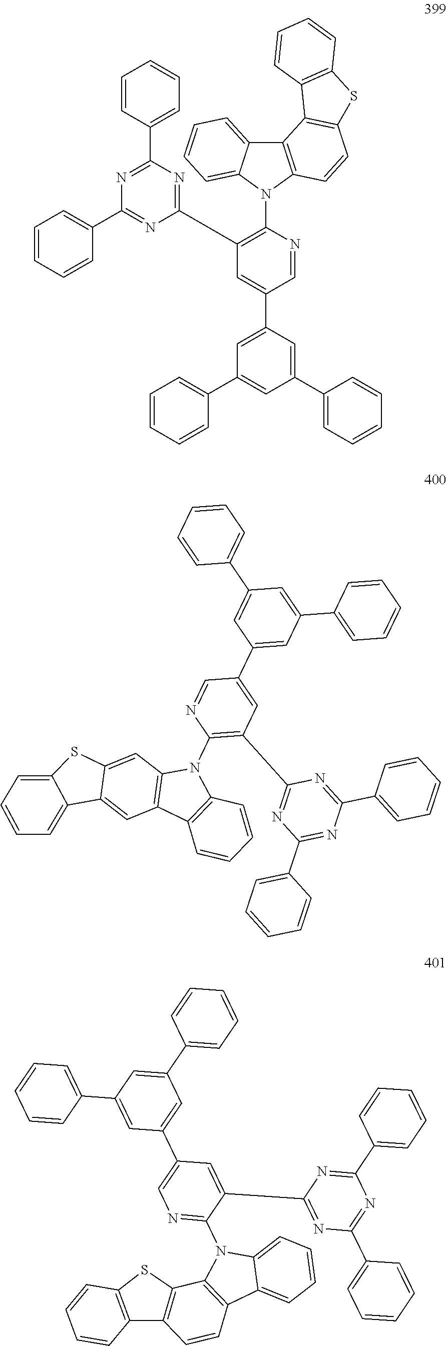 [SMILES: c1ccc(-c2cc(-c3ccccc3)cc(-c3cnc(-n4c5ccccc5c5c6c(ccc54)sc4ccccc46)c(Cc4nc(-c5ccccc5)nc(-c5ccccc5)n4)c3)c2)cc1 ; c1ccc(-c2cc(-c3ccccc3)cc(-c3cnc(-n4c5ccccc5c5cc6c(cc54)sc4ccccc46)c(Cc4nc(-c5ccccc5)nc(-c5ccccc5)n4)c3)c2)cc1 ; c1ccc(-c2cc(-c3ccccc3)cc(-c3cnc(-n4c5ccccc5c5ccc6c7ccccc7sc6c54)c(-c4nc(-c5ccccc5)nc(-c5ccccc5)n4)c3)c2)cc1]

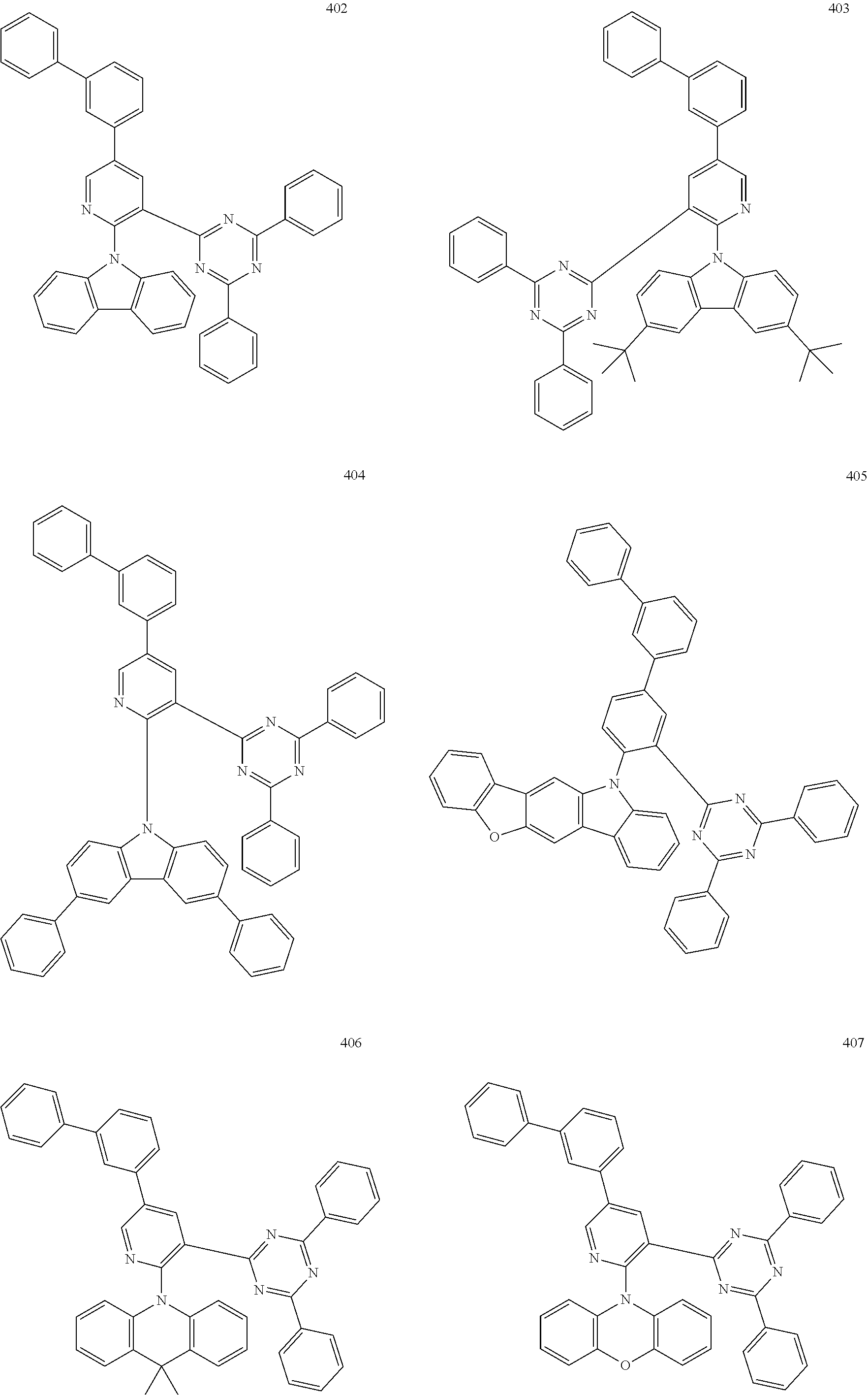

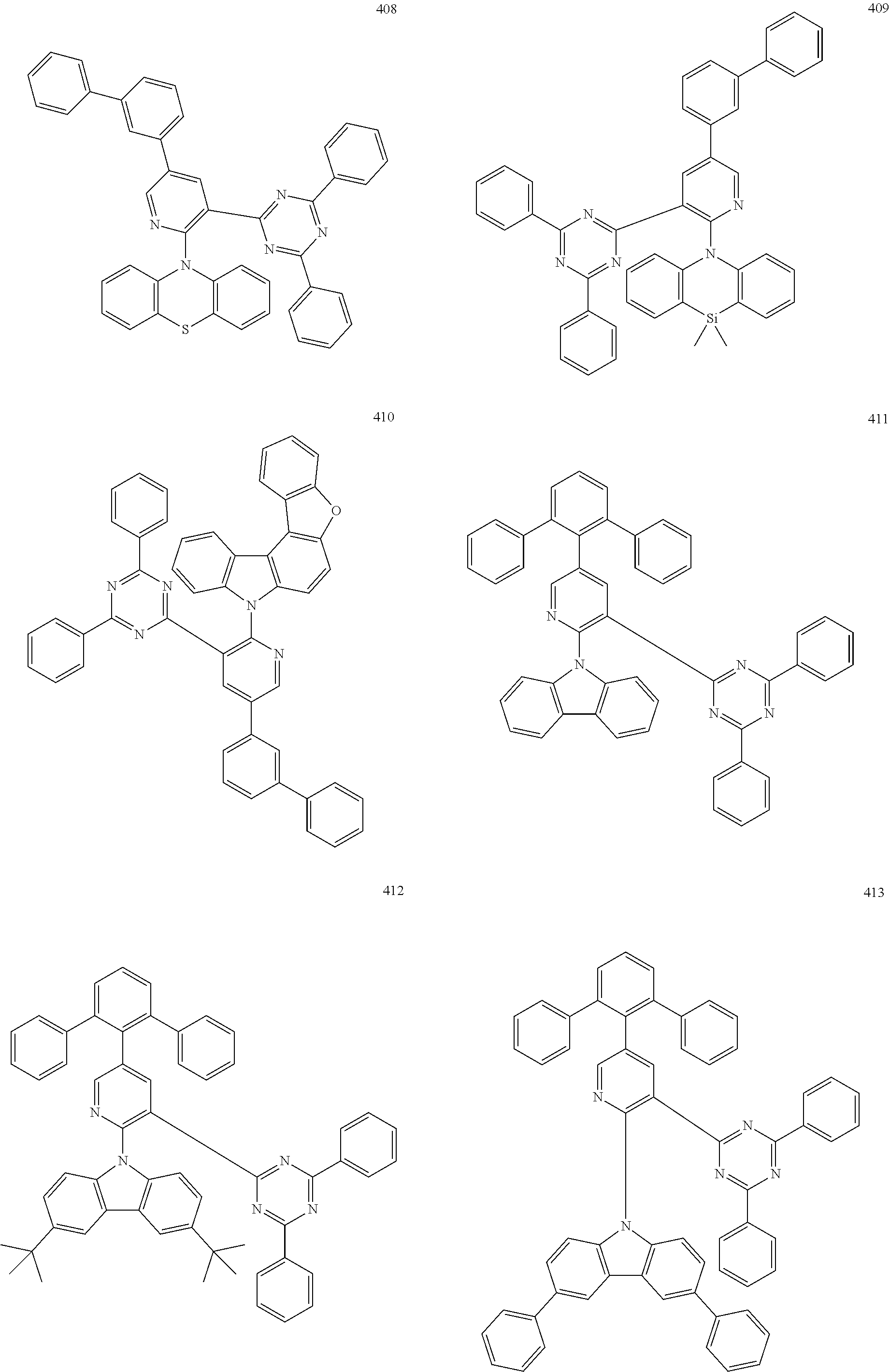

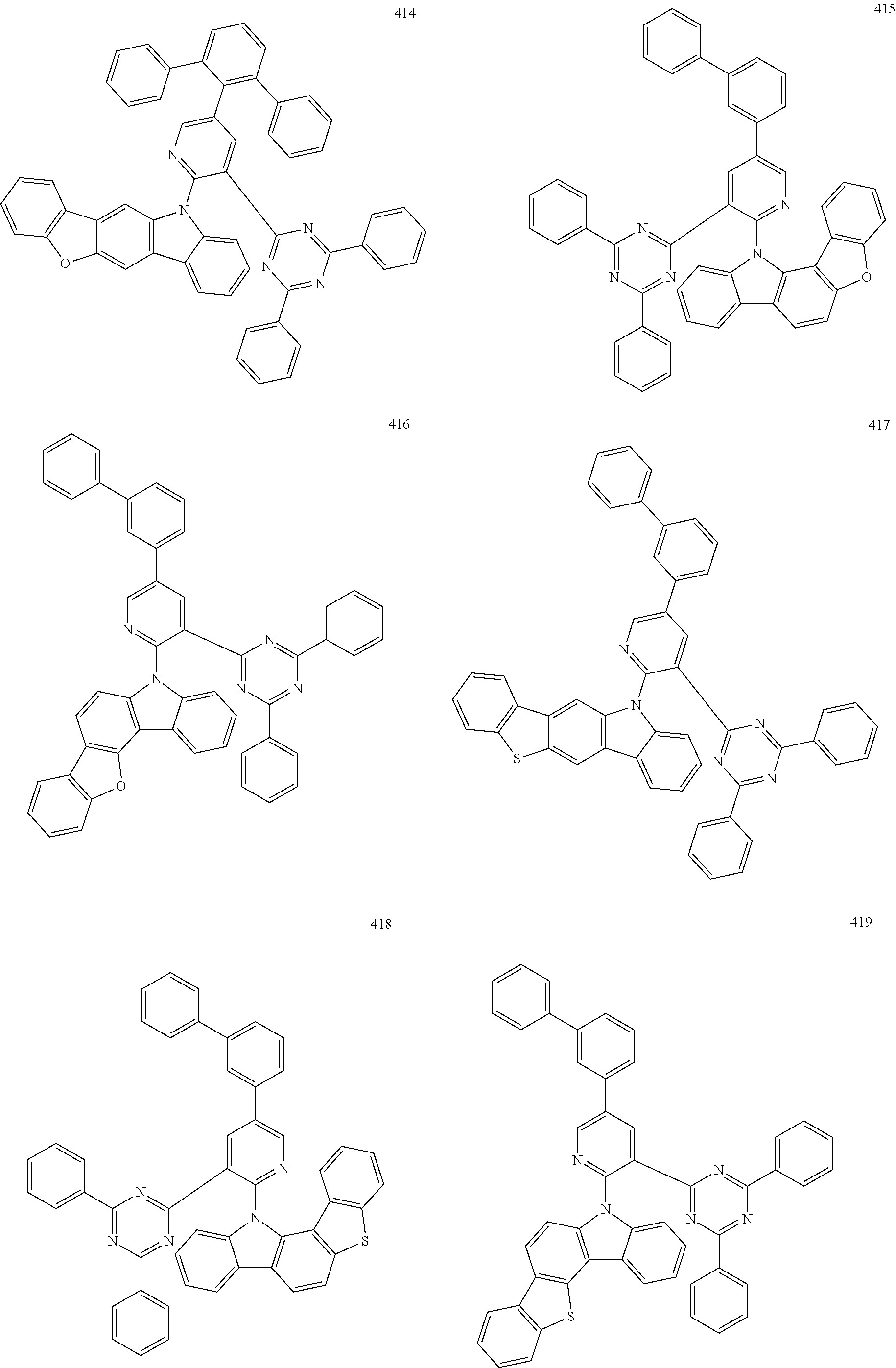

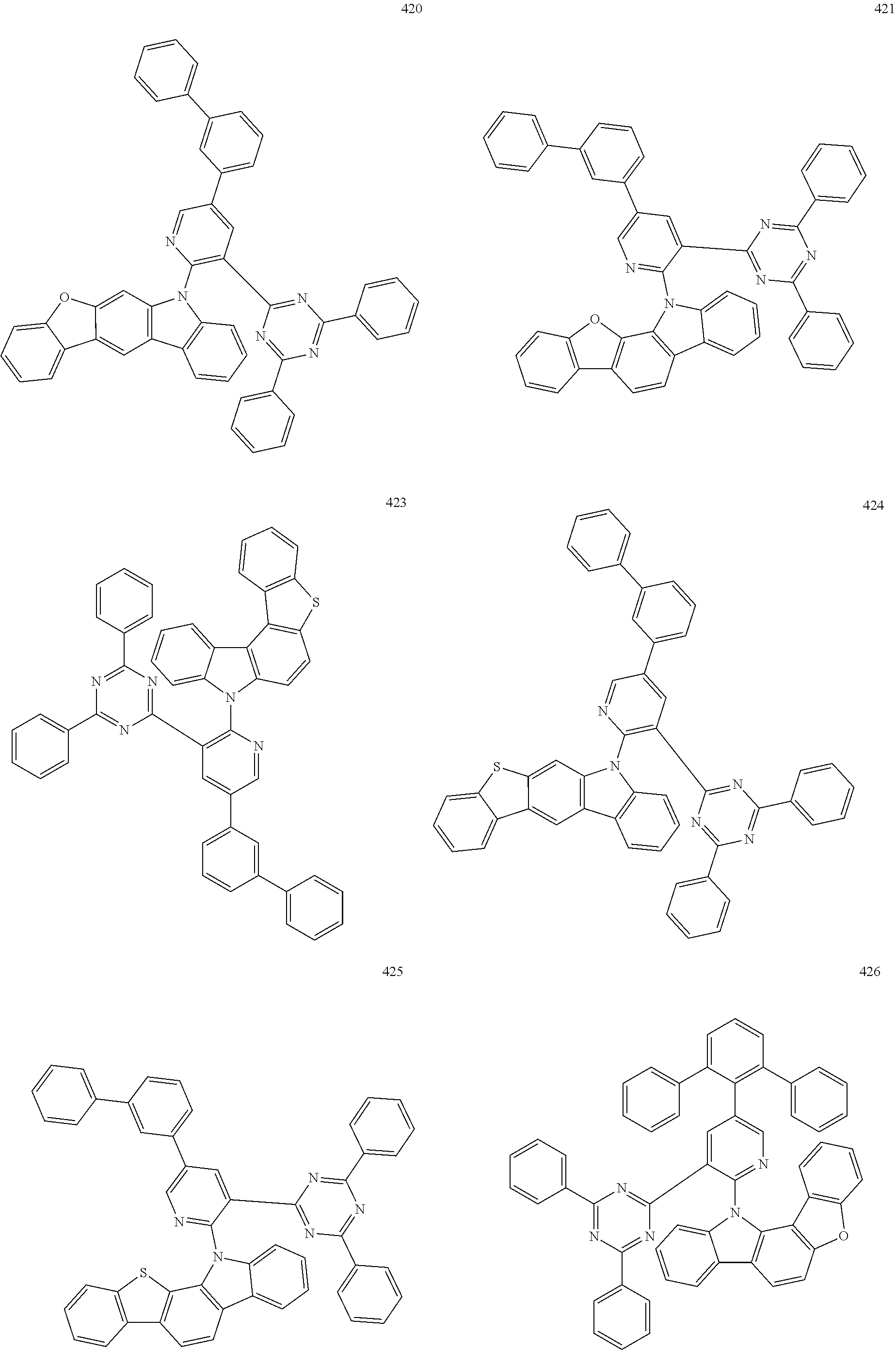

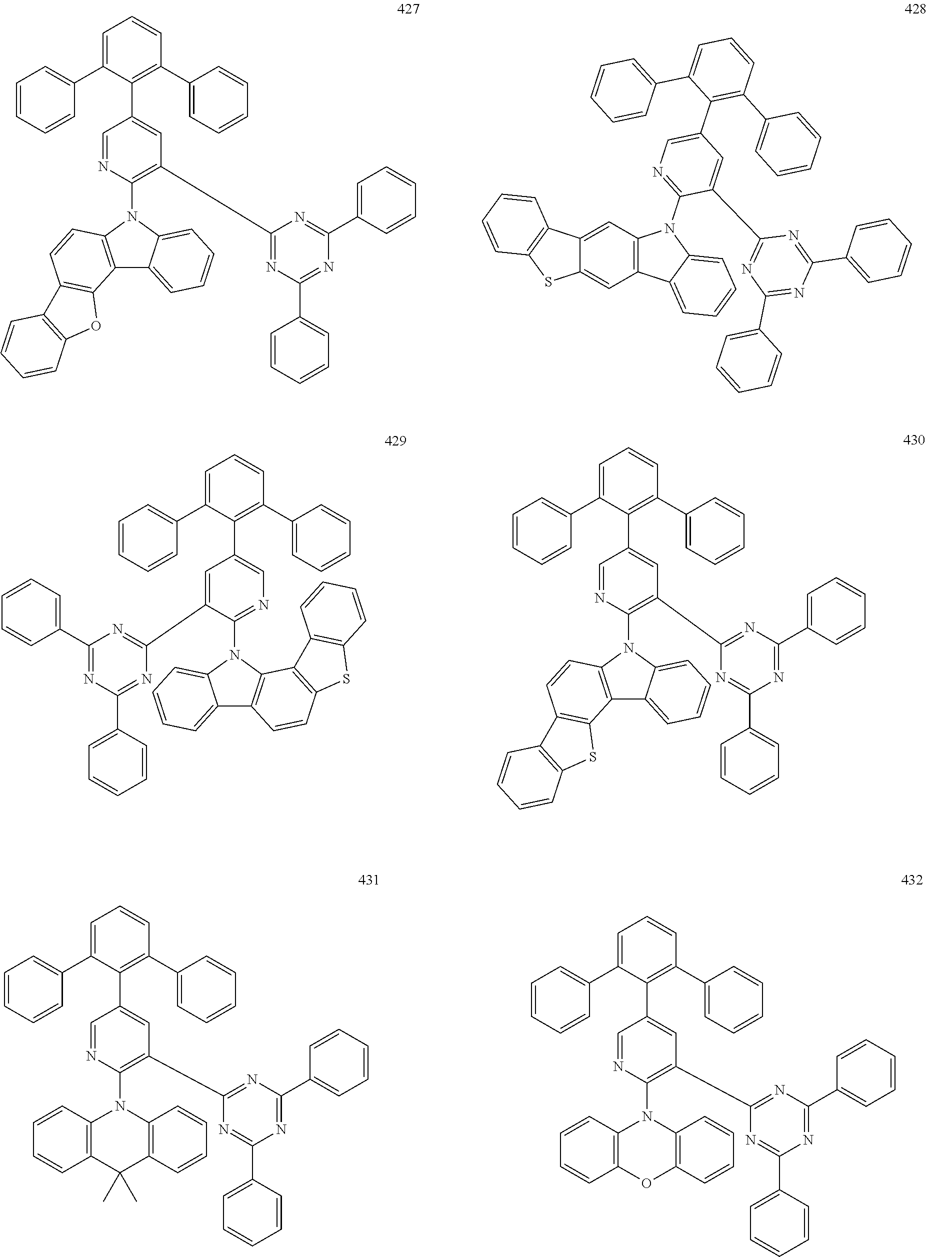

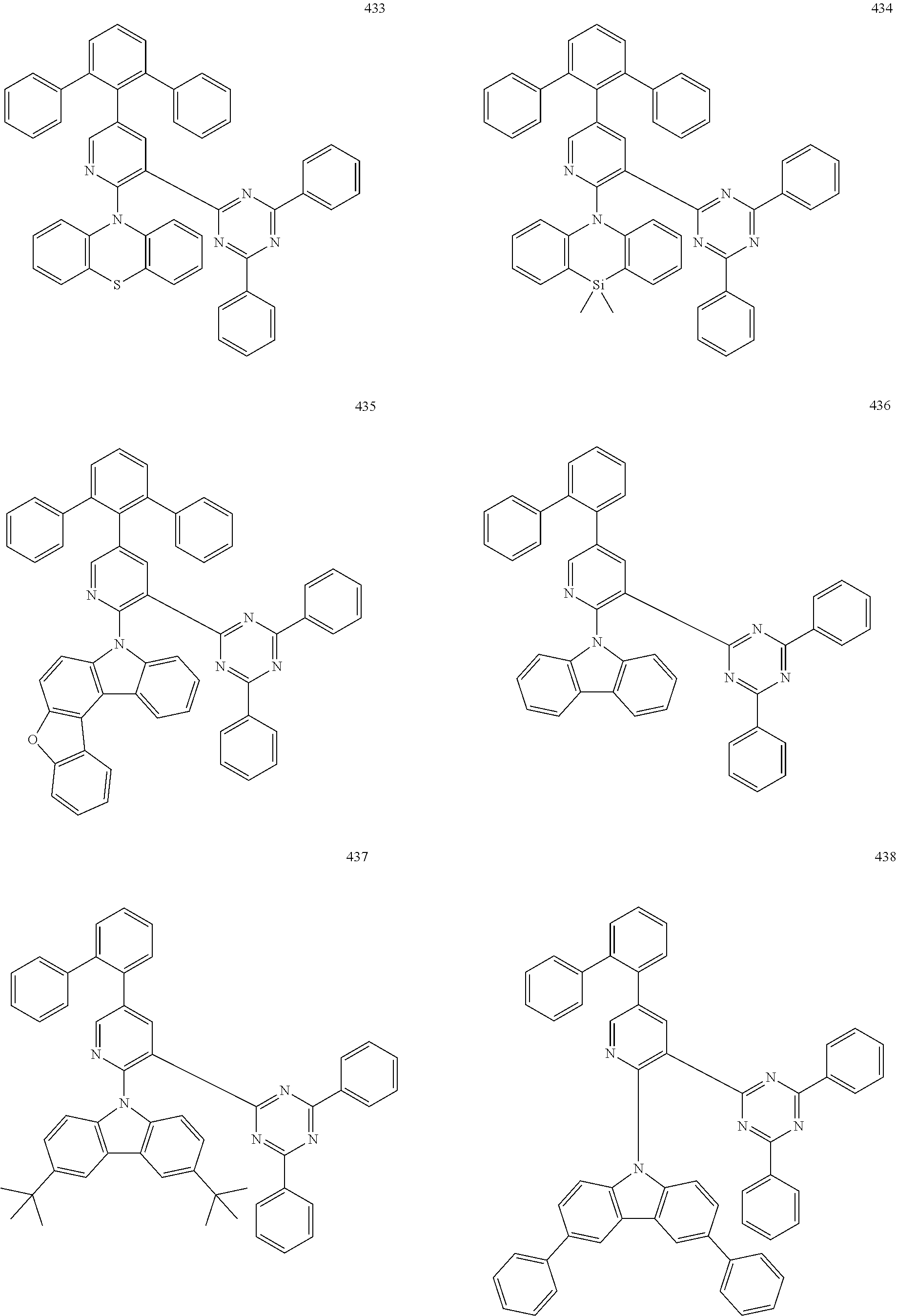

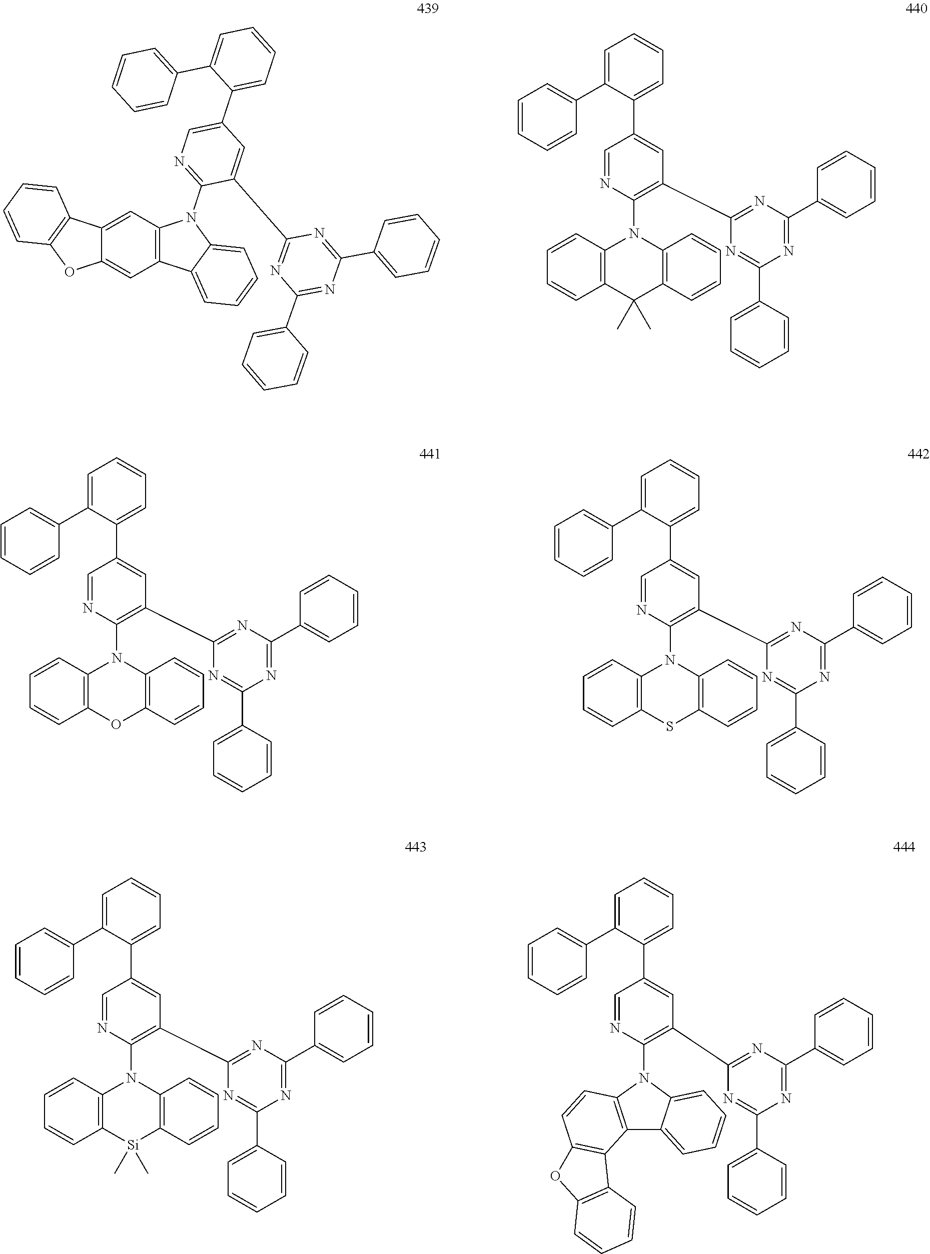

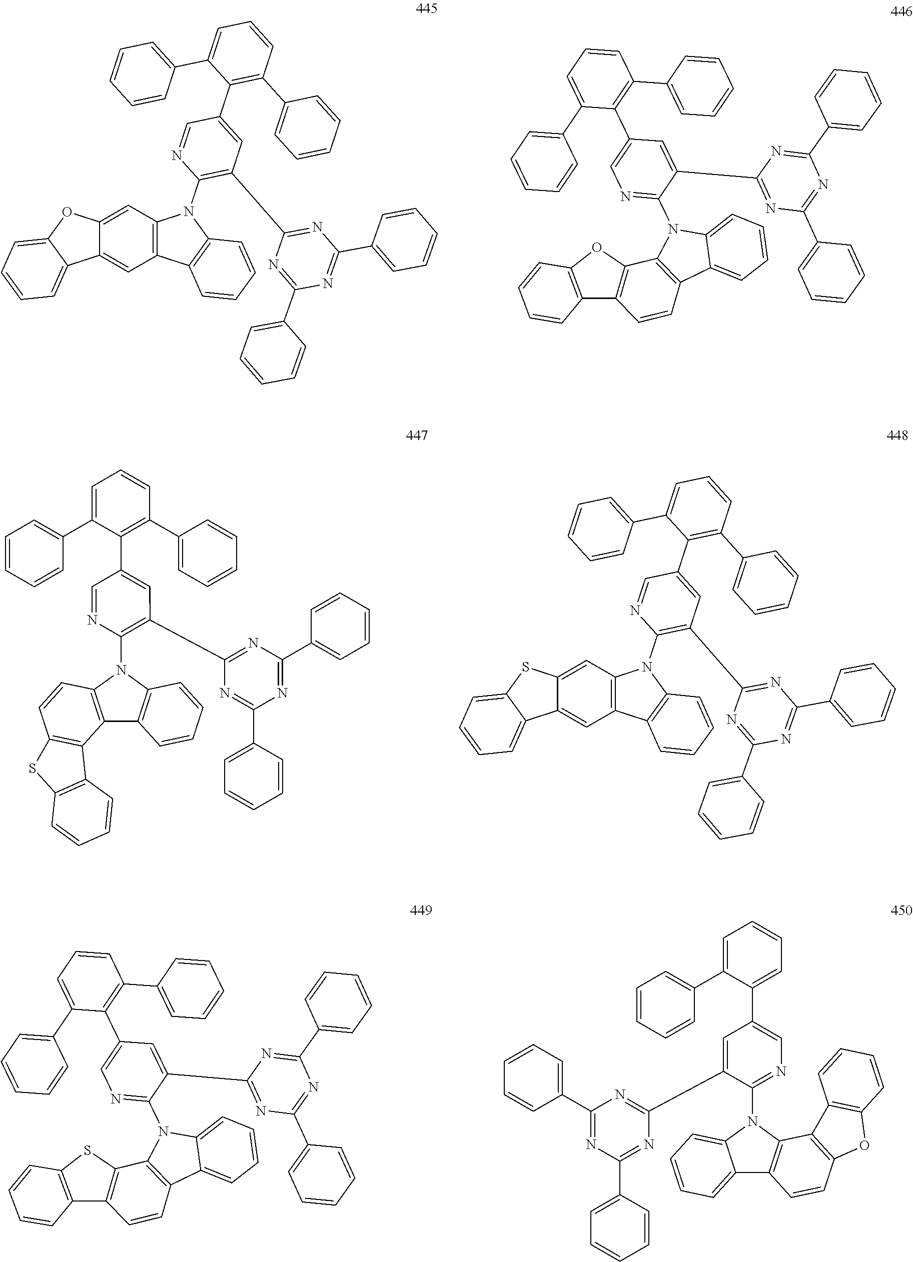

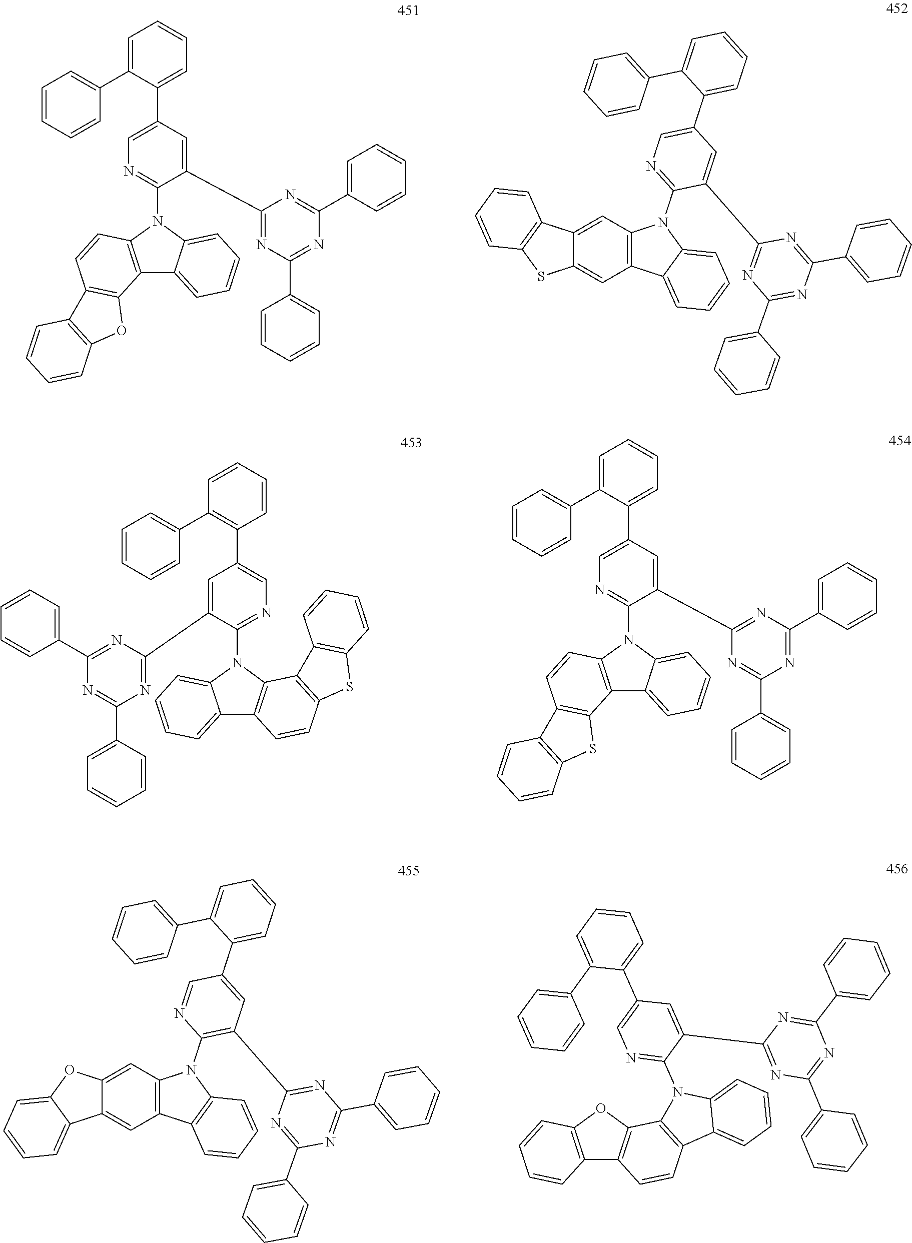

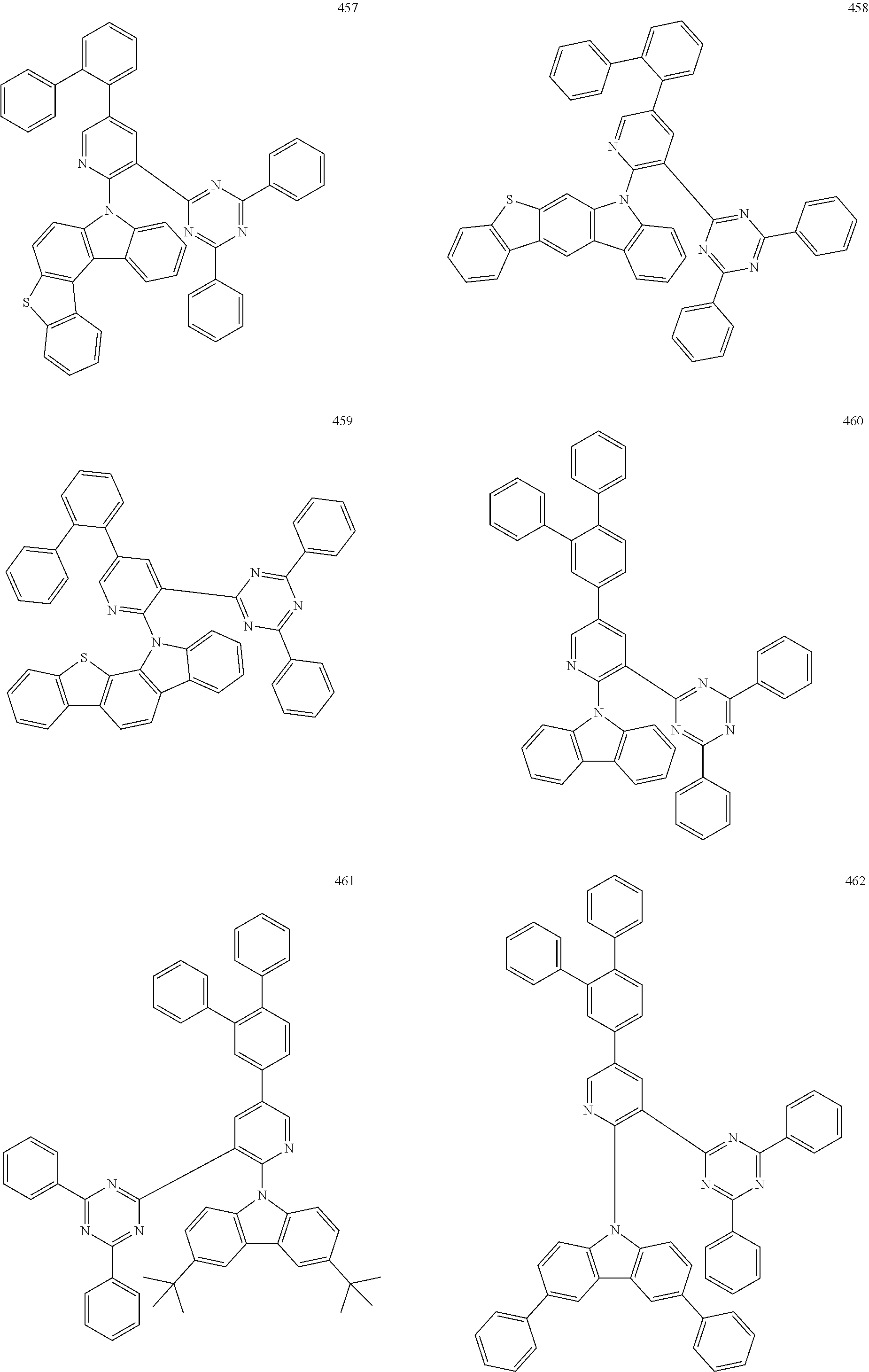

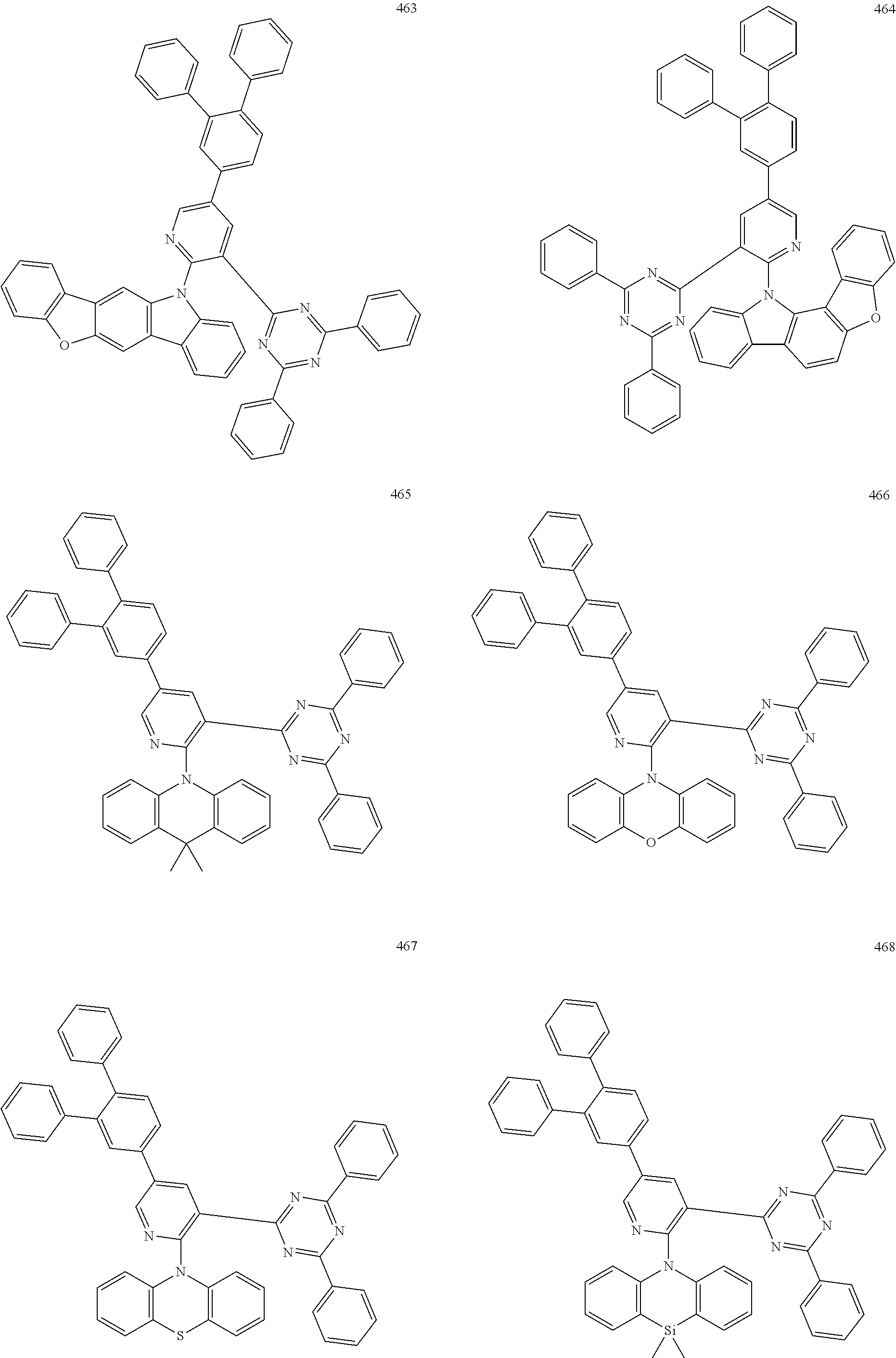

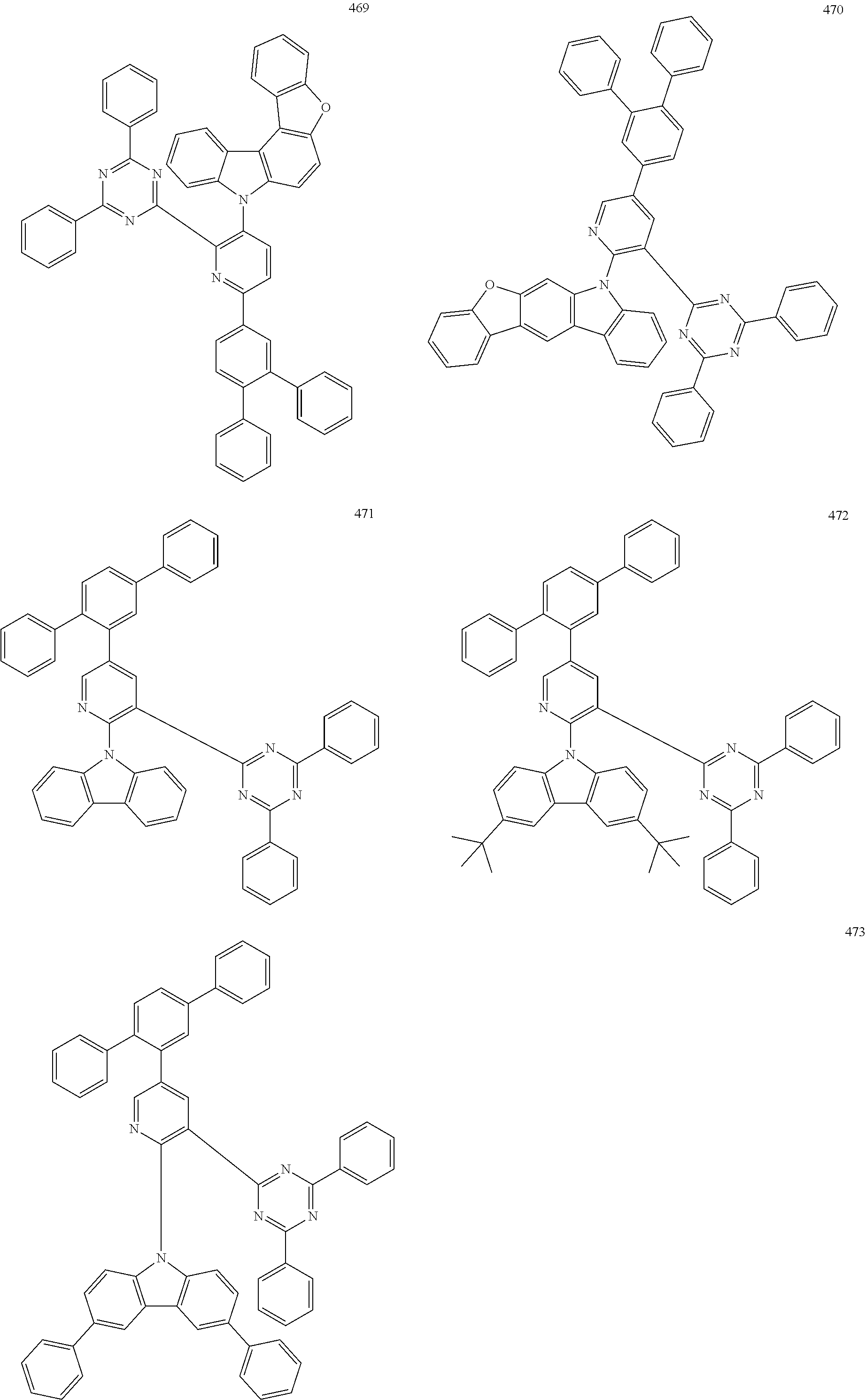

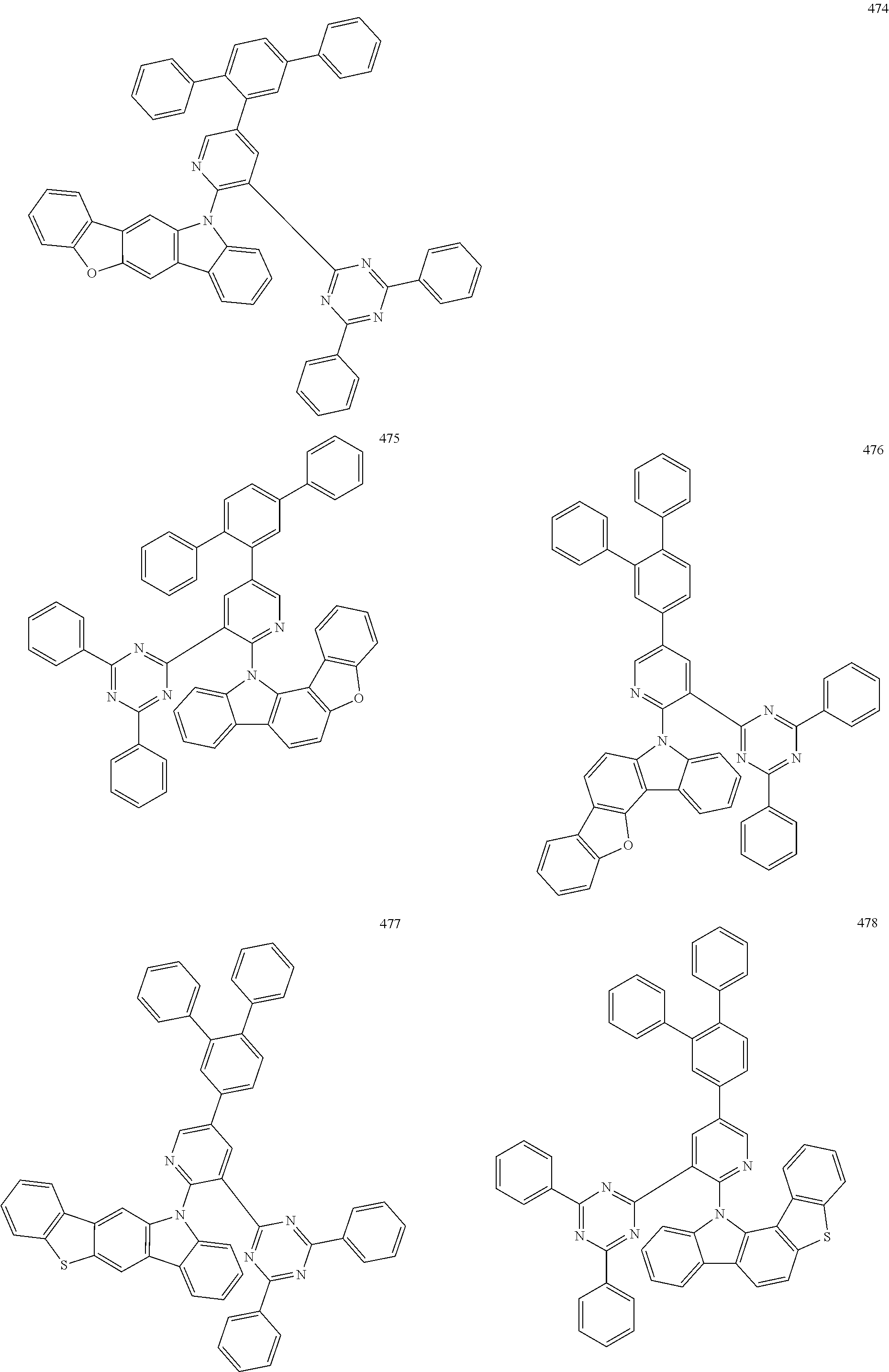

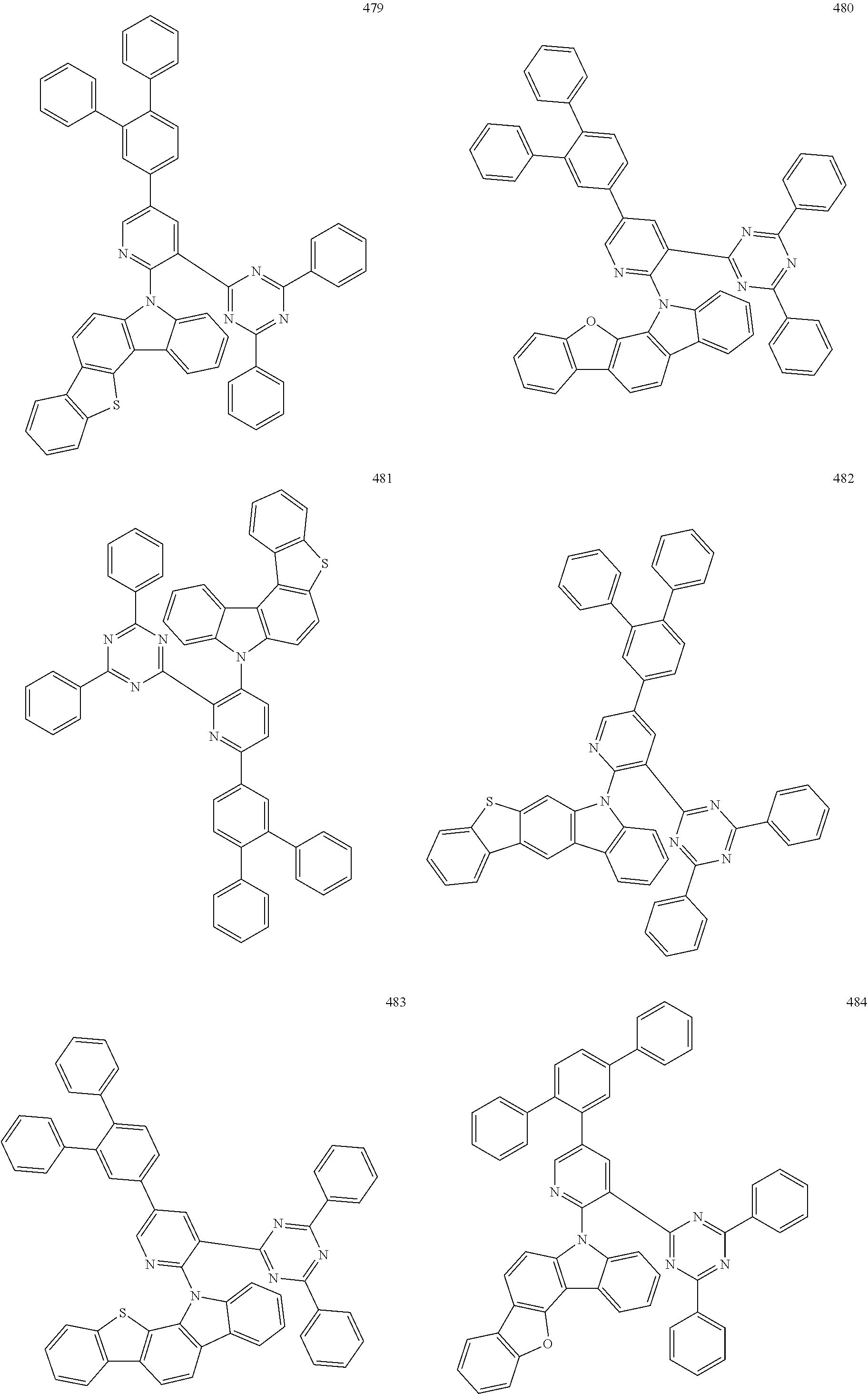

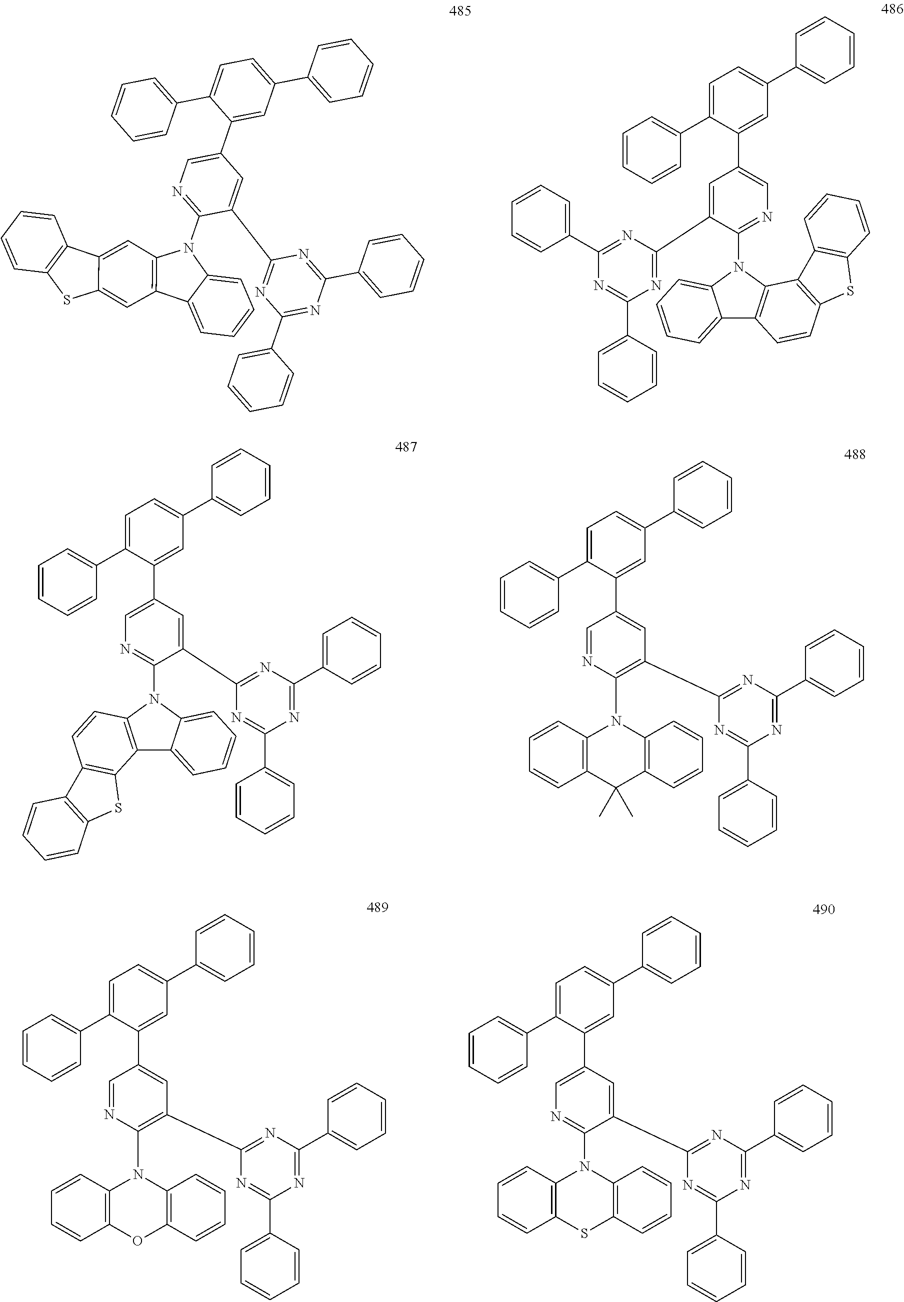

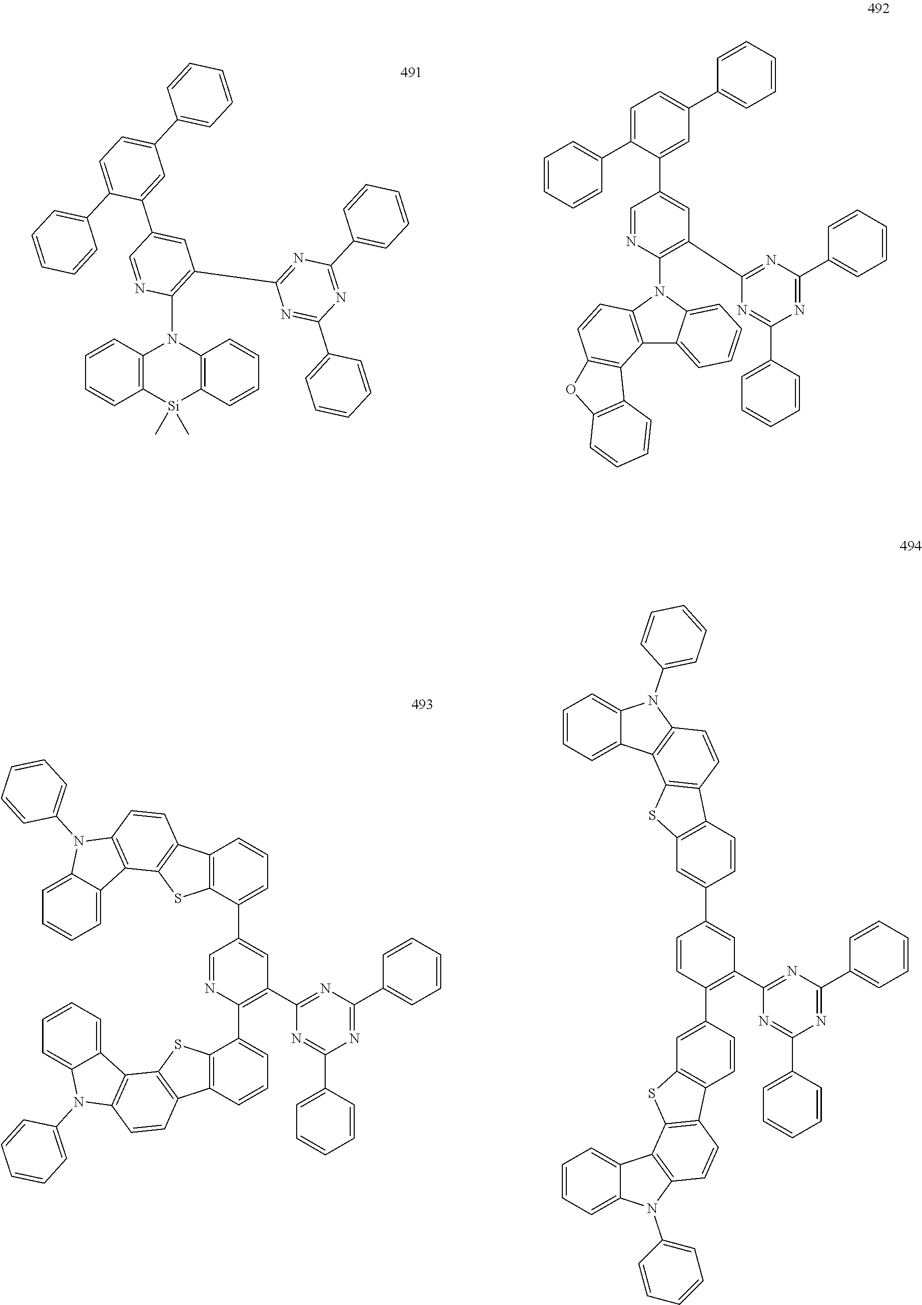 [SMILES: C[Si]1(C)c2ccccc2N(c2ncc(-c3cc(-c4ccccc4)ccc3-c3ccccc3)cc2-c2nc(-c3ccccc3)nc(-c3ccccc3)n2)c2ccccc21 ; c1ccc(-c2ccc(-c3ccccc3)c(-c3cnc(-n4c5ccccc5c5c6c(ccc54)oc4ccccc46)c(-c4nc(-c5ccccc5)nc(-c5ccccc5)n4)c3)c2)cc1 ; c1ccc(-c2nc(-c3ccccc3)nc(-c3cc(-c4cccc5c4sc4c5ccc5c4c4ccccc4n5-c4ccccc4)cnc3-c3cccc4c3sc3c4ccc4c3c3ccccc3n4-c3ccccc3)n2)cc1 ; c1ccc(-c2nc(-c3ccccc3)nc(-c3cc(-c4ccc5c(c4)sc4c5ccc5c4c4ccccc4n5-c4ccccc4)ccc3-c3ccc4c(c3)sc3c4ccc4c3c3ccccc3n4-c3ccccc3)n2)cc1]

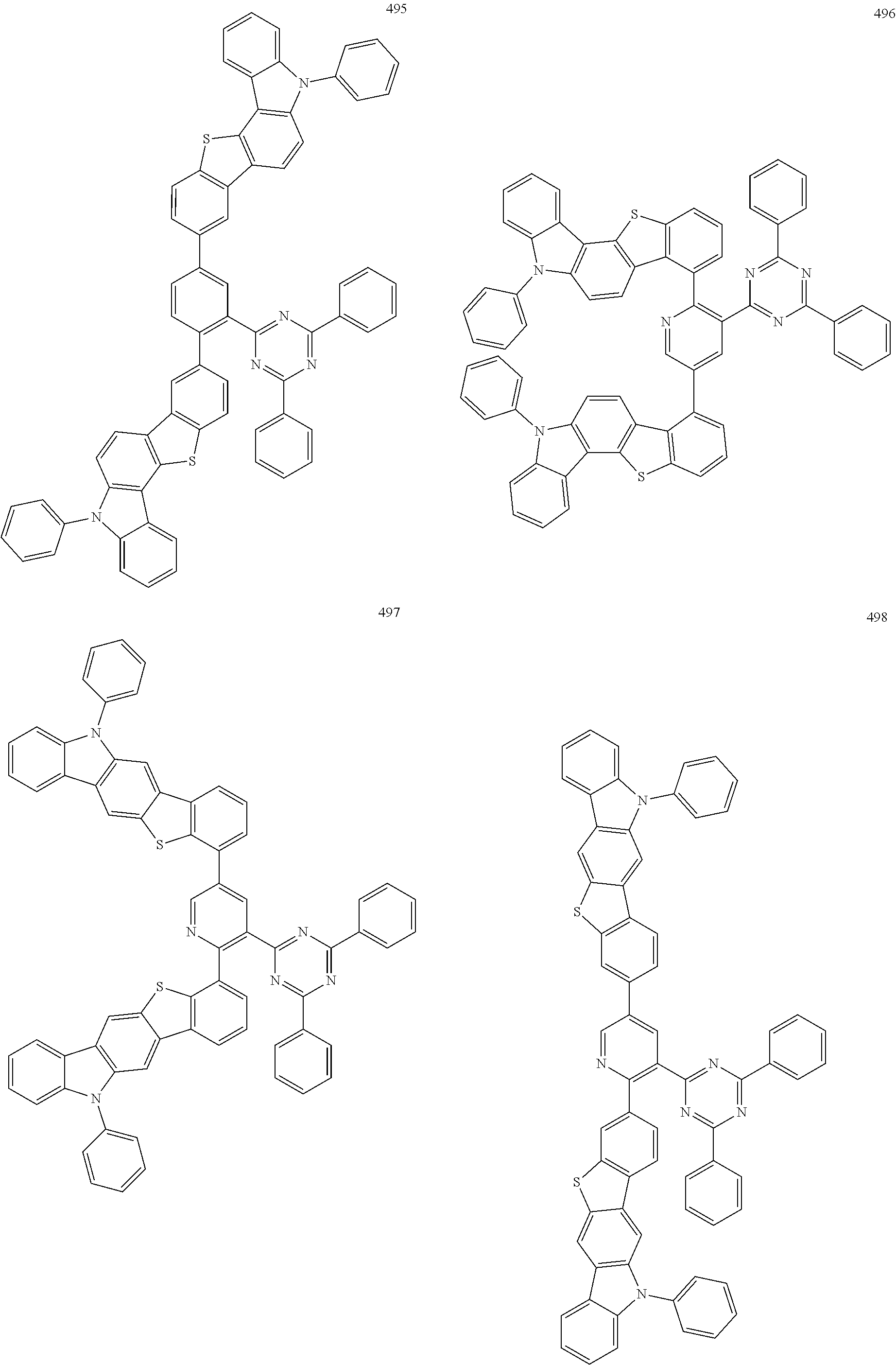

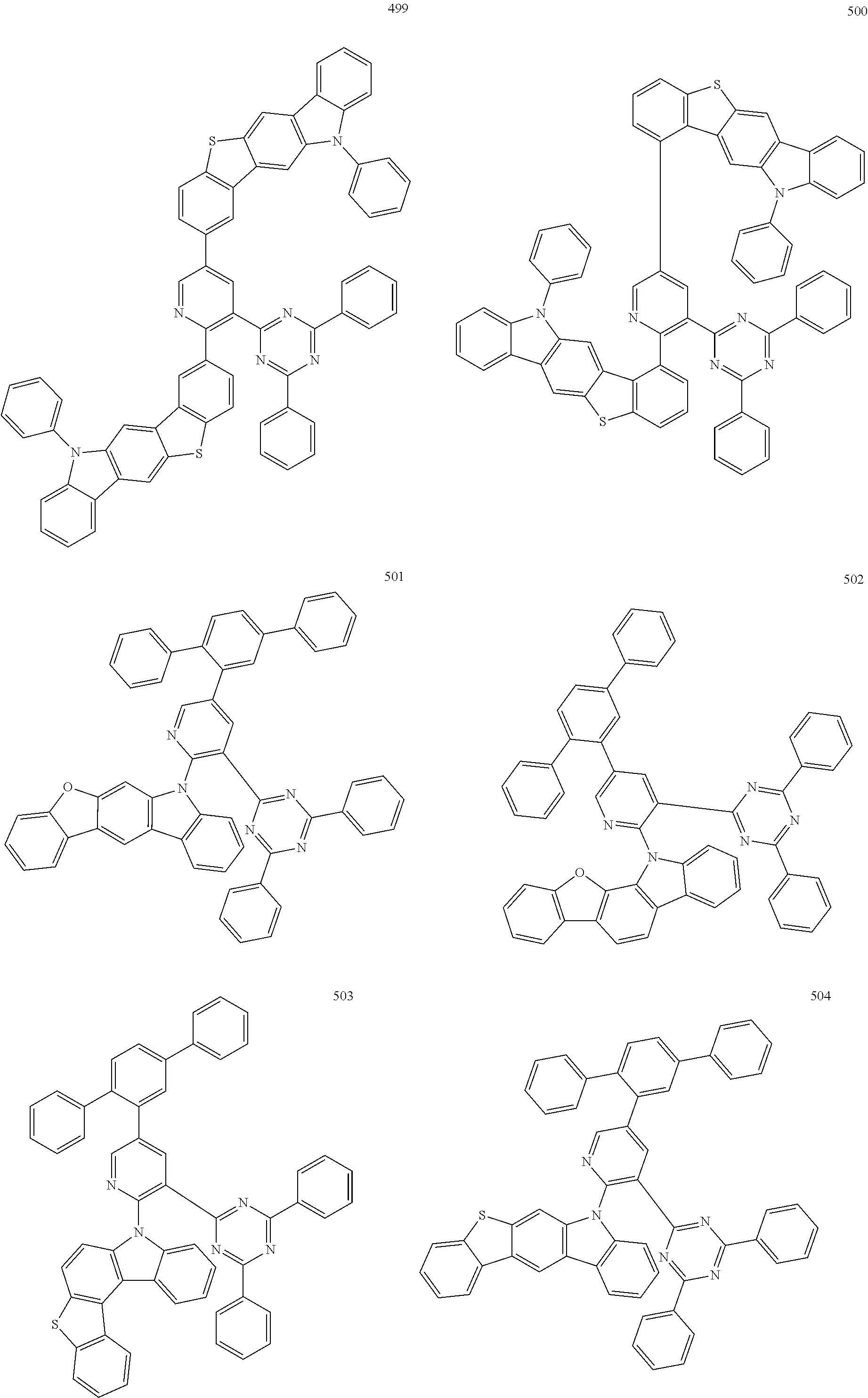

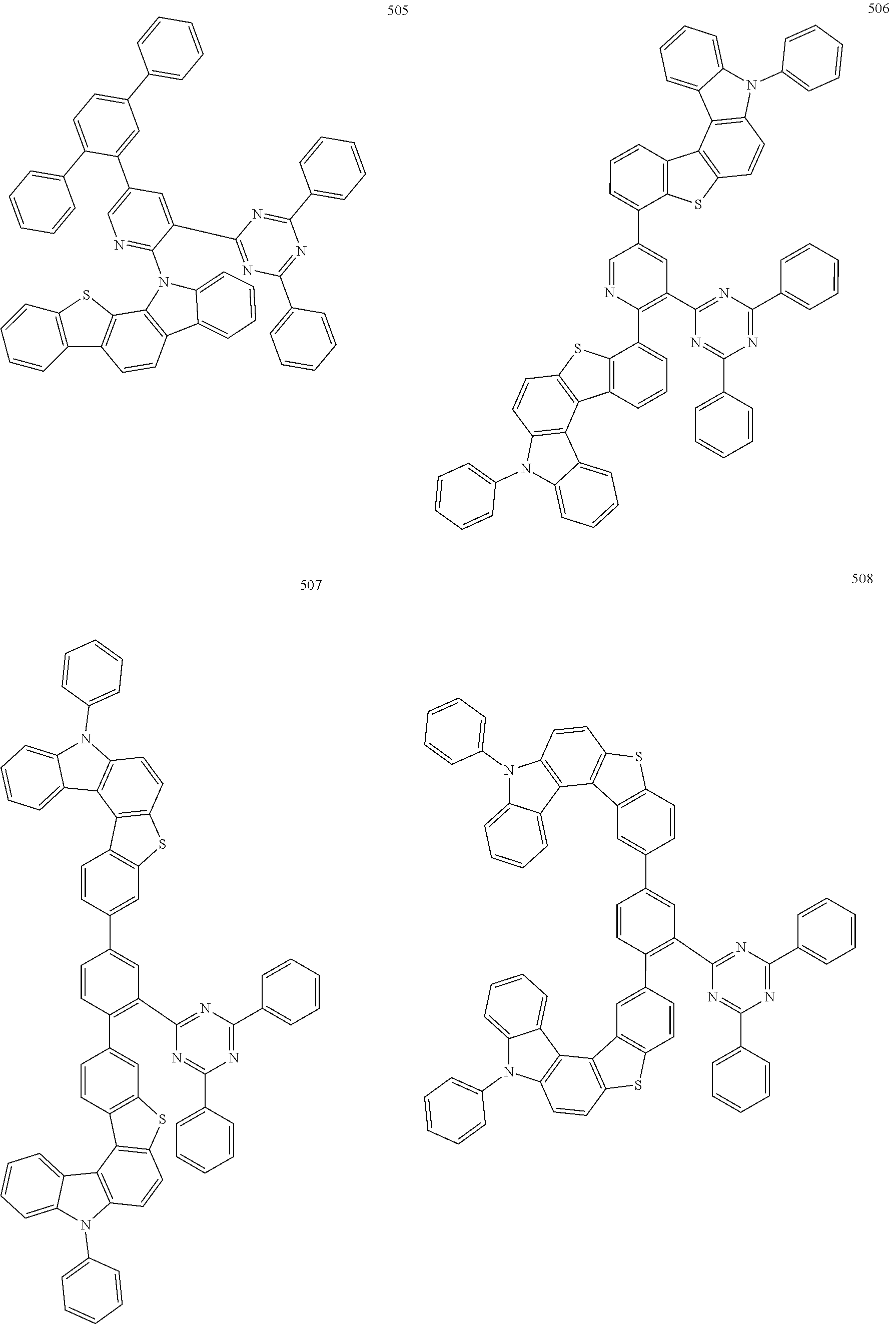

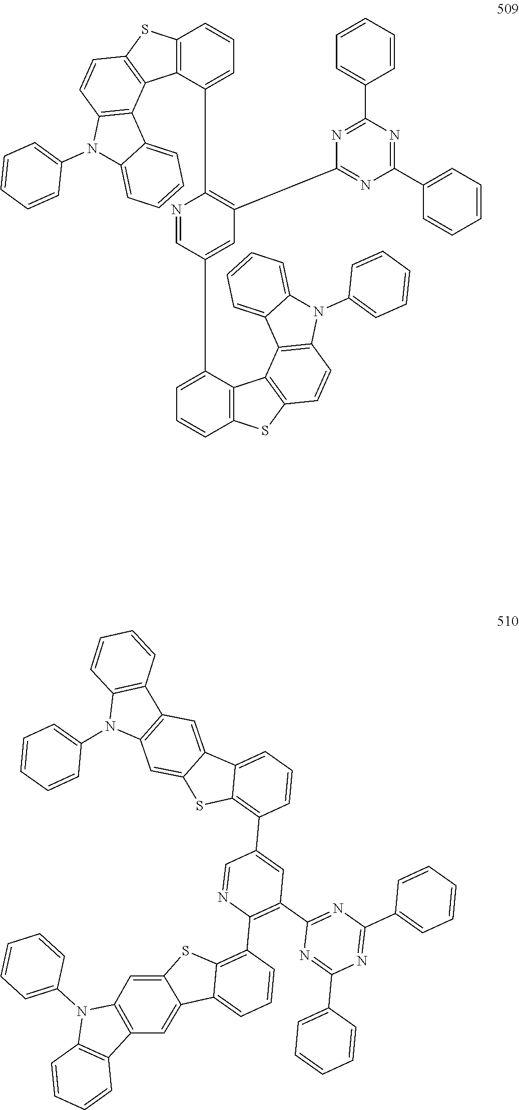 [SMILES: c1ccc(-c2nc(-c3ccccc3)nc(-c3cc(-c4cccc5sc6ccc7c(c8ccccc8n7-c7ccccc7)c6c45)cnc3-c3cccc4sc5ccc6c(c7ccccc7n6-c6ccccc6)c5c34)n2)cc1 ; c1ccc(-c2nc(-c3ccccc3)nc(-c3cc(-c4cccc5c4sc4cc6c(cc45)c4ccccc4n6-c4ccccc4)cnc3-c3cccc4c3sc3cc5c(cc34)c3ccccc3n5-c3ccccc3)n2)cc1]

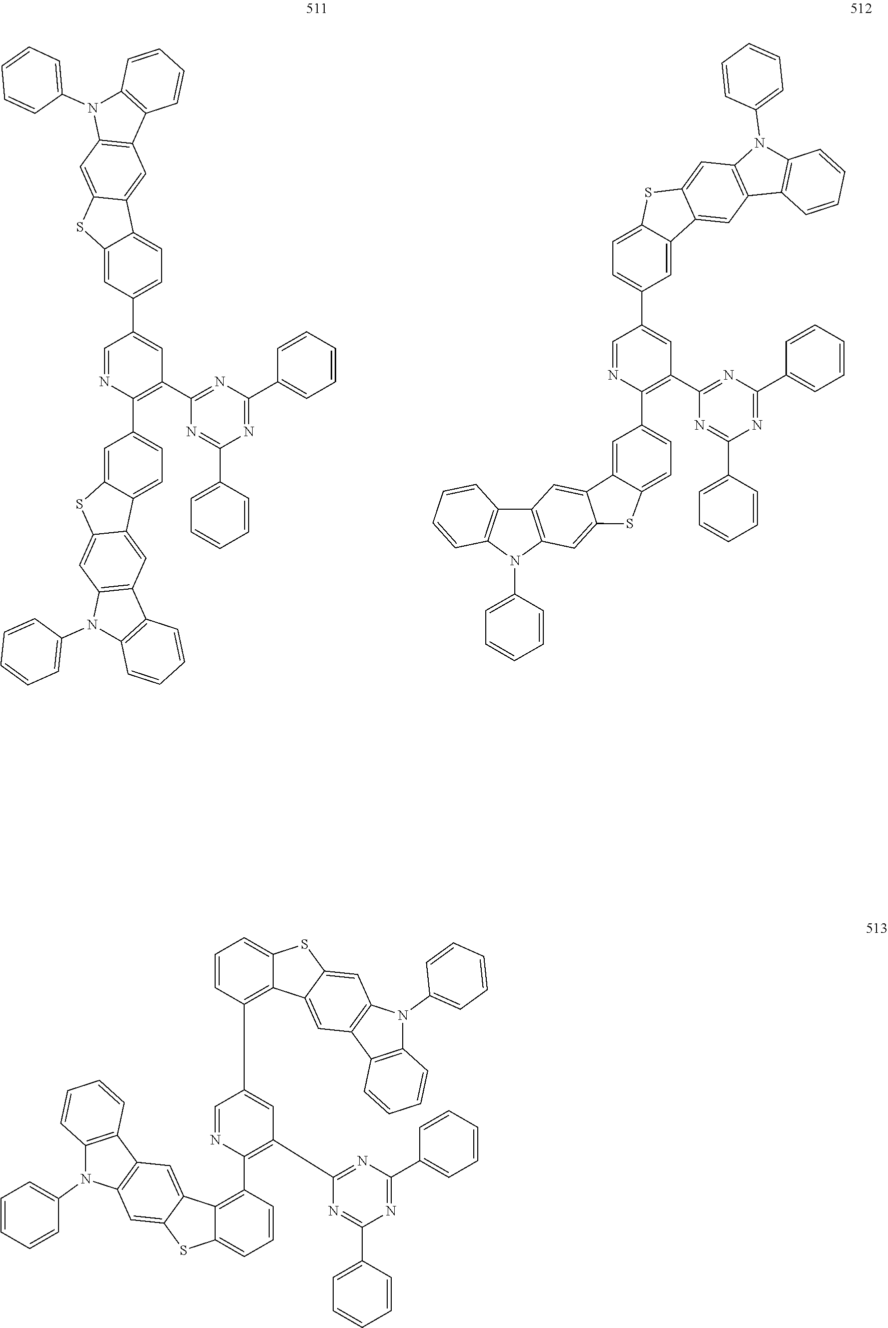 [SMILES: c1ccc(-c2nc(-c3ccccc3)nc(-c3cc(-c4ccc5c(c4)sc4cc6c(cc45)c4ccccc4n6-c4ccccc4)cnc3-c3ccc4c(c3)sc3cc5c(cc34)c3ccccc3n5-c3ccccc3)n2)cc1 ; c1ccc(-c2nc(-c3ccccc3)nc(-c3cc(-c4ccc5sc6cc7c(cc6c5c4)c4ccccc4n7-c4ccccc4)cnc3-c3ccc4sc5cc6c(cc5c4c3)c3ccccc3n6-c3ccccc3)n2)cc1 ; c1ccc(-c2nc(-c3ccccc3)nc(-c3cc(-c4cccc5sc6cc7c(cc6c45)c4ccccc4n7-c4ccccc4)cnc3-c3cccc4sc5cc6c(cc5c34)c3ccccc3n6-c3ccccc3)n2)cc1]

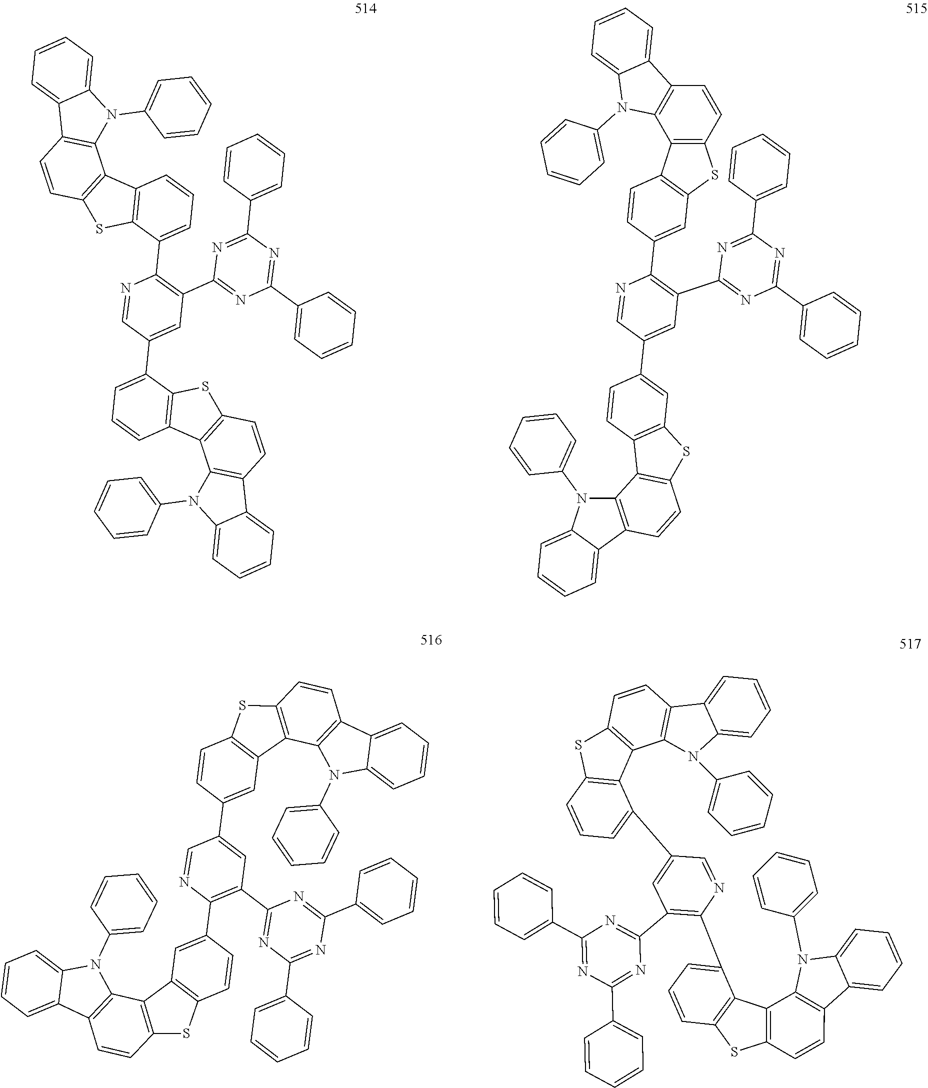

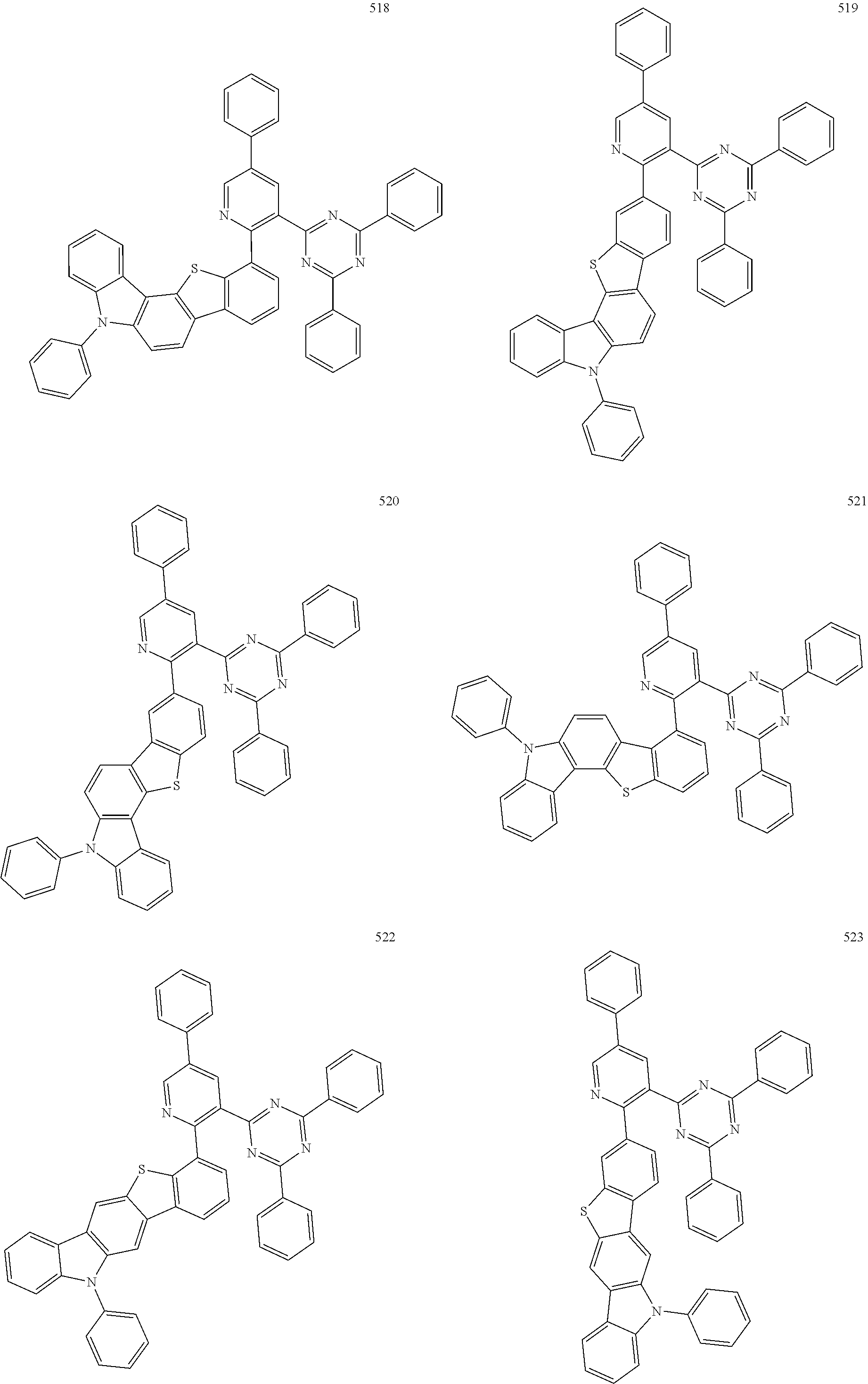

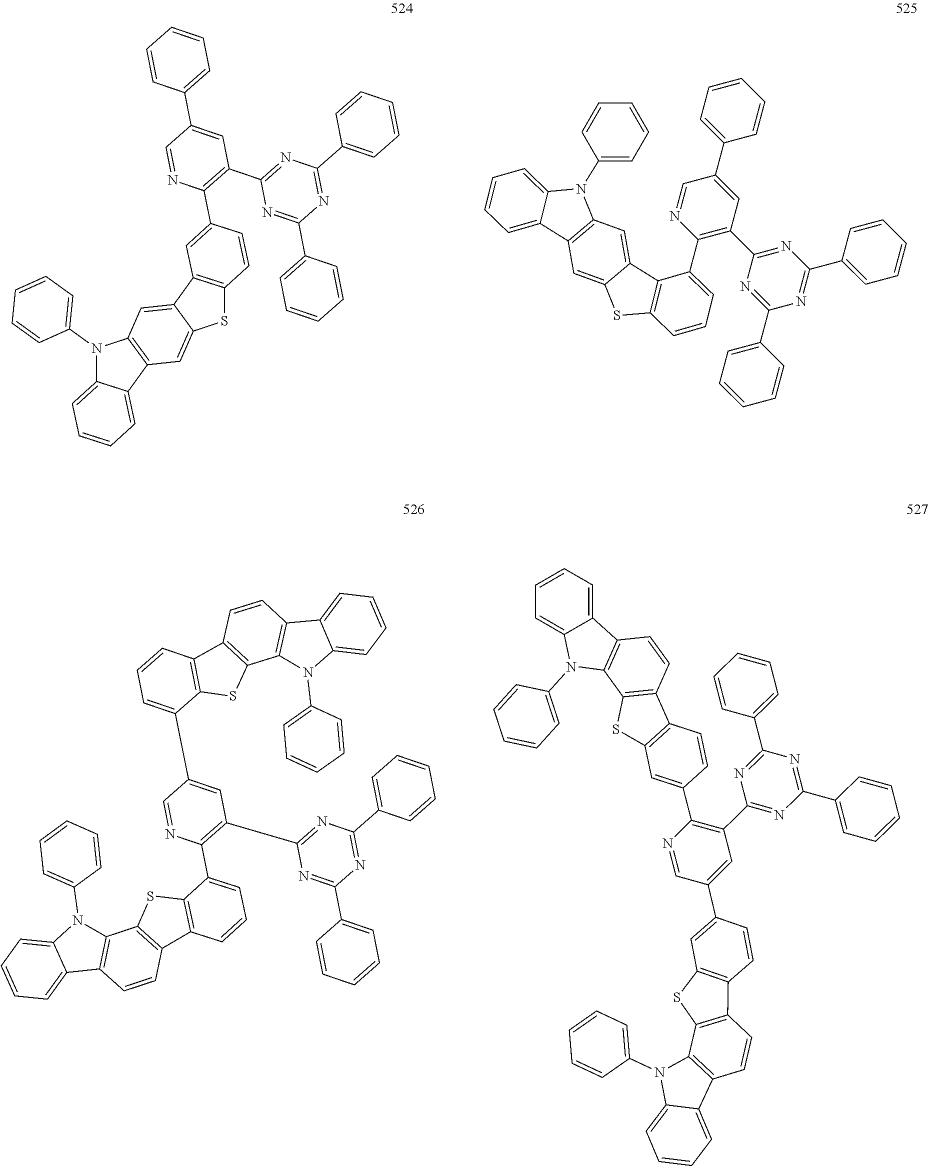 [SMILES: c1ccc(-c2cnc(-c3ccc4sc5cc6c7ccccc7n(-c7ccccc7)c6cc5c4c3)c(-c3nc(-c4ccccc4)nc(-c4ccccc4)n3)c2)cc1 ; c1ccc(-c2cnc(-c3cccc4sc5cc6c7ccccc7n(-c7ccccc7)c6cc5c34)c(-c3nc(-c4ccccc4)nc(-c4ccccc4)n3)c2)cc1 ; c1ccc(-c2nc(-c3ccccc3)nc(-c3cc(-c4cccc5c4sc4c5ccc5c6ccccc6n(-c6ccccc6)c54)cnc3-c3cccc4c3sc3c4ccc4c5ccccc5n(-c5ccccc5)c43)n2)cc1 ; c1ccc(-c2nc(-c3ccccc3)nc(-c3cc(-c4ccc5c(c4)sc4c5ccc5c6ccccc6n(-c6ccccc6)c54)cnc3-c3ccc4c(c3)sc3c4ccc4c5ccccc5n(-c5ccccc5)c43)n2)cc1]

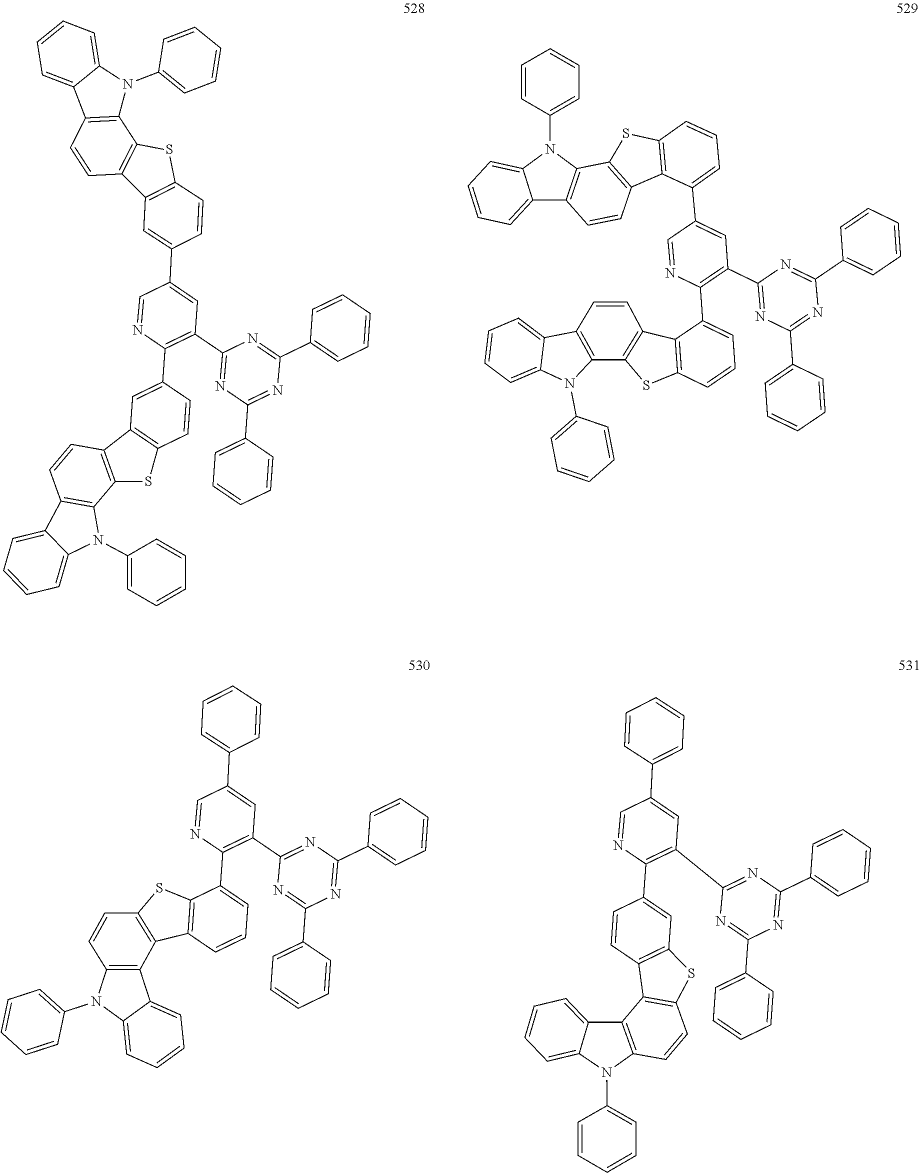 [SMILES: c1ccc(-c2nc(-c3ccccc3)nc(-c3cc(-c4ccc5sc6c(ccc7c8ccccc8n(-c8ccccc8)c76)c5c4)cnc3-c3ccc4sc5c(ccc6c7ccccc7n(-c7ccccc7)c65)c4c3)n2)cc1 ; c1ccc(-c2nc(-c3ccccc3)nc(-c3cc(-c4cccc5sc6c(ccc7c8ccccc8n(-c8ccccc8)c76)c45)cnc3-c3cccc4sc5c(ccc6c7ccccc7n(-c7ccccc7)c65)c34)n2)cc1 ; c1ccc(-c2cnc(-c3cccc4c3sc3ccc5c(c6ccccc6n5-c5ccccc5)c34)c(-c3nc(-c4ccccc4)nc(-c4ccccc4)n3)c2)cc1 ; c1ccc(-c2cnc(-c3ccc4c(c3)sc3ccc5c(c6ccccc6n5-c5ccccc5)c34)c(-c3nc(-c4ccccc4)nc(-c4ccccc4)n3)c2)cc1]

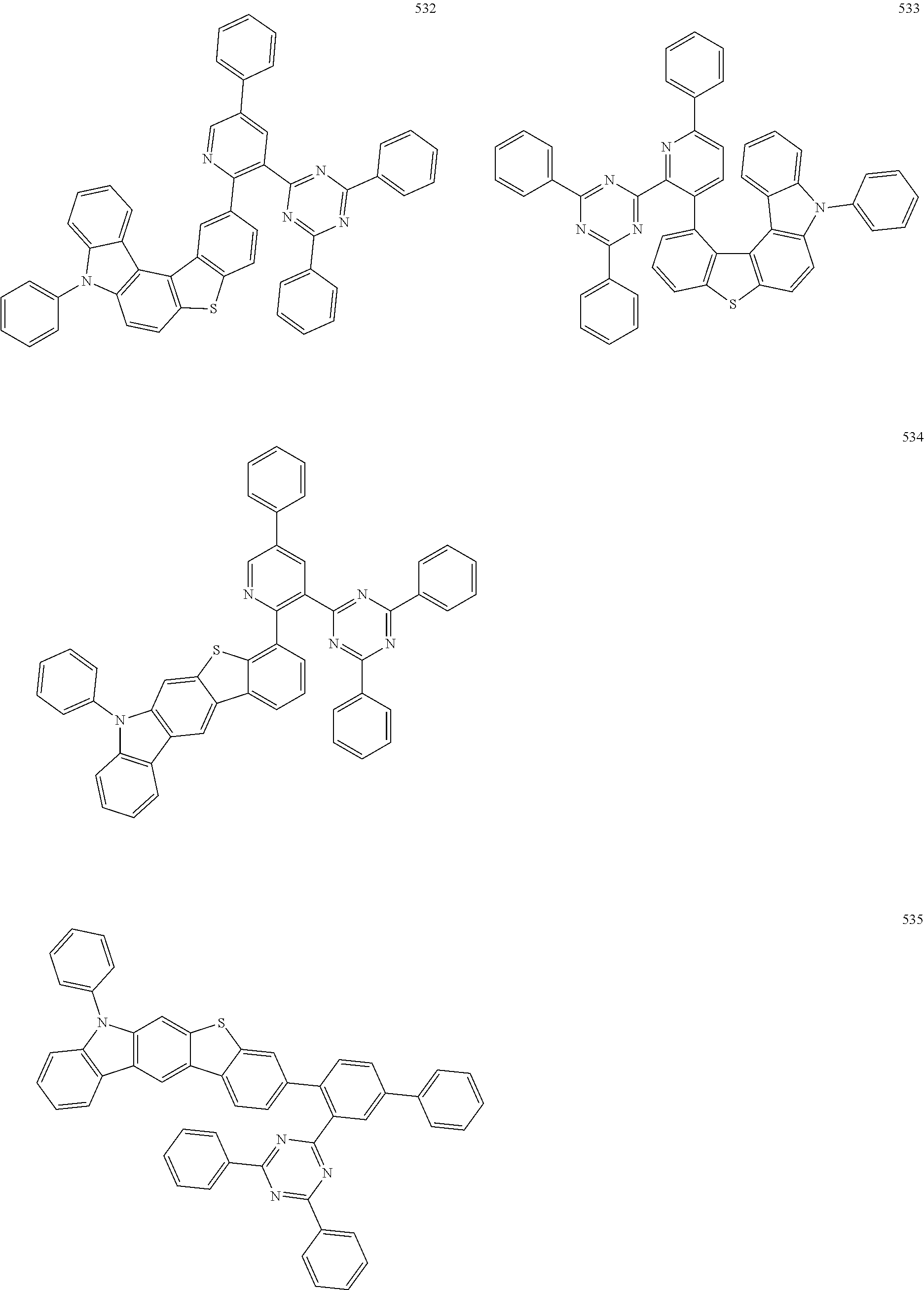 [SMILES: c1ccc(-c2cnc(-c3ccc4sc5ccc6c(c7ccccc7n6-c6ccccc6)c5c4c3)c(-c3nc(-c4ccccc4)nc(-c4ccccc4)n3)c2)cc1 ; c1ccc(-c2ccc(-c3cccc4sc5ccc6c(c7ccccc7n6-c6ccccc6)c5c34)c(-c3nc(-c4ccccc4)nc(-c4ccccc4)n3)n2)cc1 ; c1ccc(-c2cnc(-c3cccc4c3sc3cc5c(cc34)c3ccccc3n5-c3ccccc3)c(-c3nc(-c4ccccc4)nc(-c4ccccc4)n3)c2)cc1 ; c1ccc(-c2ccc(-c3ccc4c(c3)sc3cc5c(cc34)c3ccccc3n5-c3ccccc3)c(-c3nc(-c4ccccc4)nc(-c4ccccc4)n3)c2)cc1]

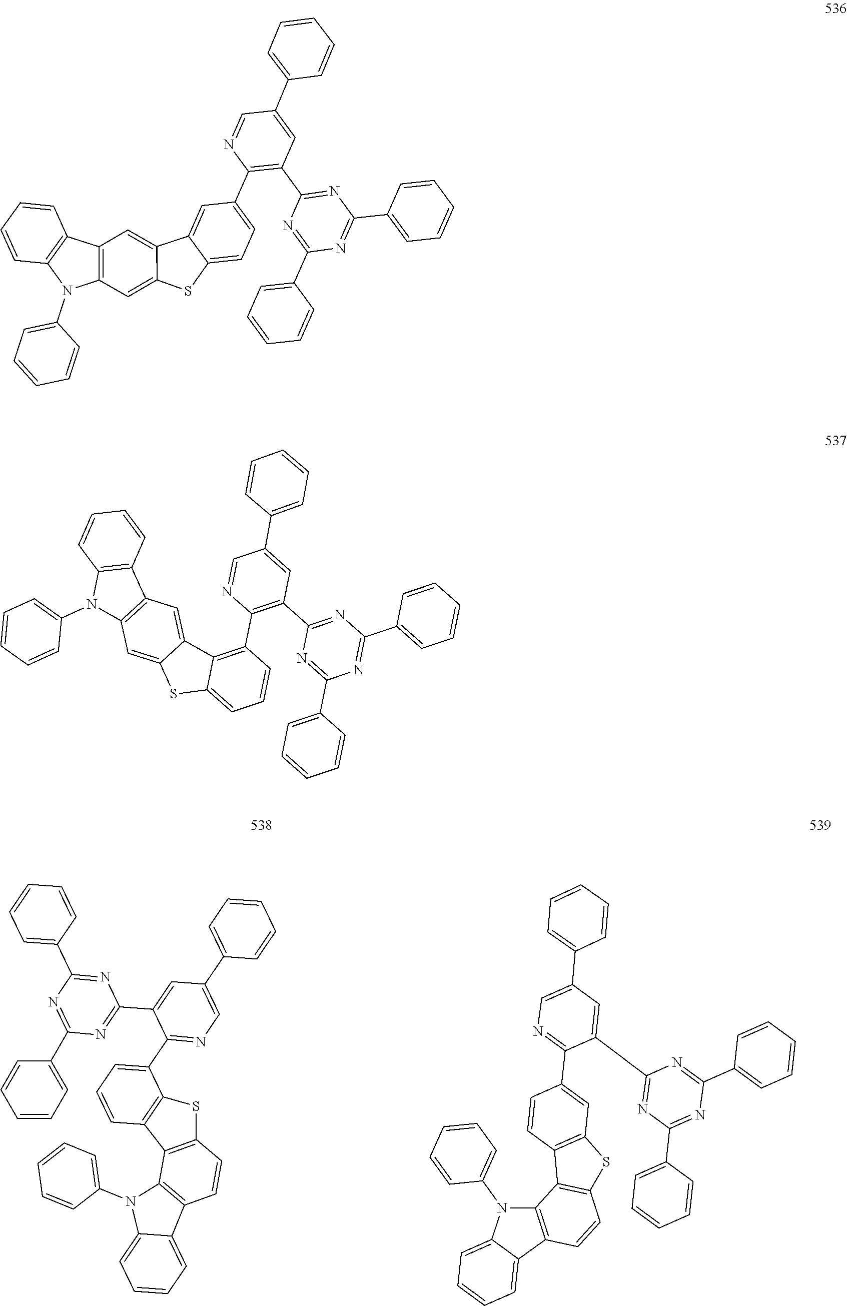 [SMILES: c1ccc(-c2cnc(-c3ccc4sc5cc6c(cc5c4c3)c3ccccc3n6-c3ccccc3)c(-c3nc(-c4ccccc4)nc(-c4ccccc4)n3)c2)cc1 ; c1ccc(-c2cnc(-c3cccc4sc5cc6c(cc5c34)c3ccccc3n6-c3ccccc3)c(-c3nc(-c4ccccc4)nc(-c4ccccc4)n3)c2)cc1 ; c1ccc(-c2cnc(-c3cccc4c3sc3ccc5c6ccccc6n(-c6ccccc6)c5c34)c(-c3nc(-c4ccccc4)nc(-c4ccccc4)n3)c2)cc1 ; c1ccc(-c2cnc(-c3ccc4c(c3)sc3ccc5c6ccccc6n(-c6ccccc6)c5c34)c(-c3nc(-c4ccccc4)nc(-c4ccccc4)n3)c2)cc1]

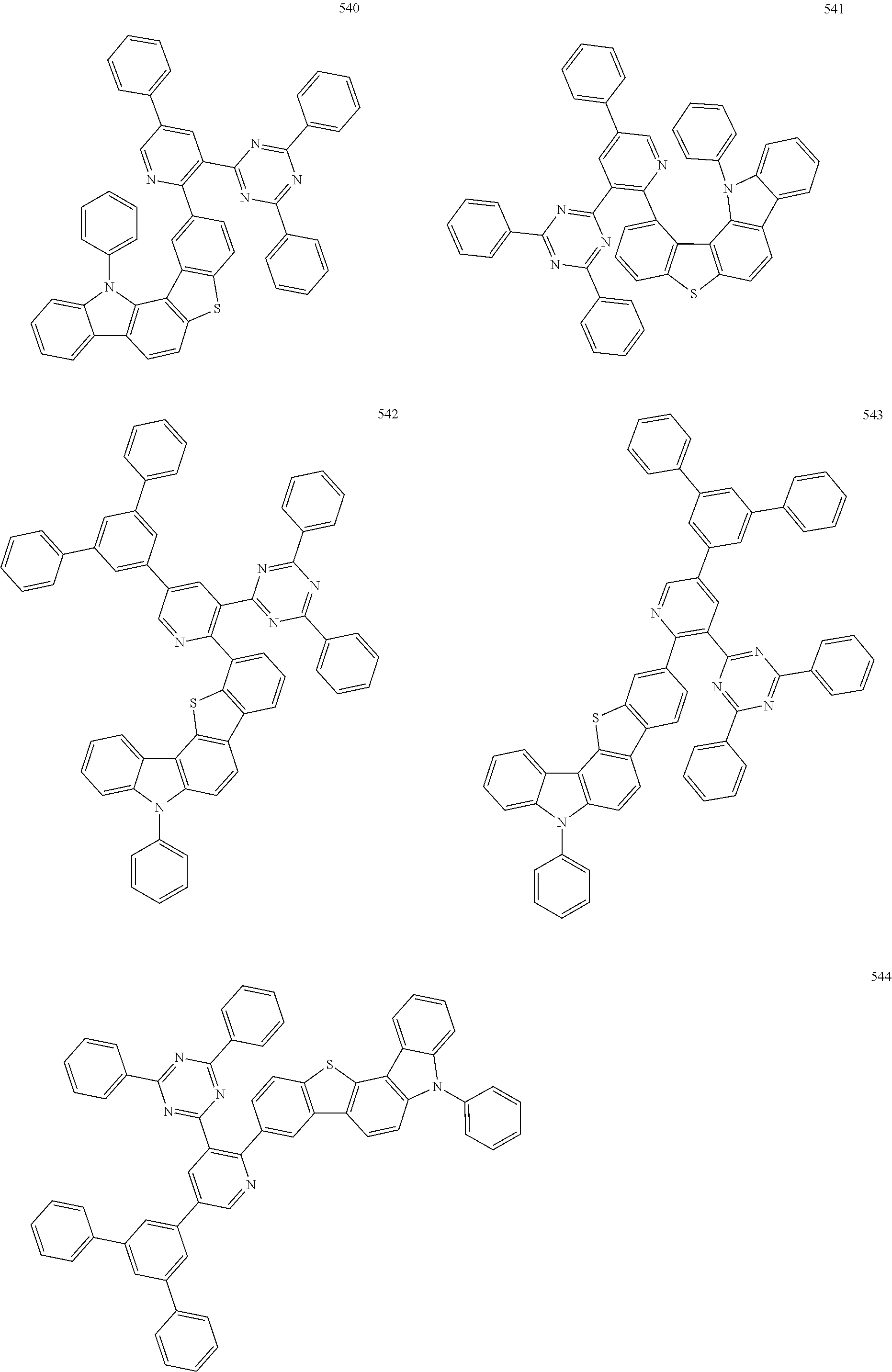

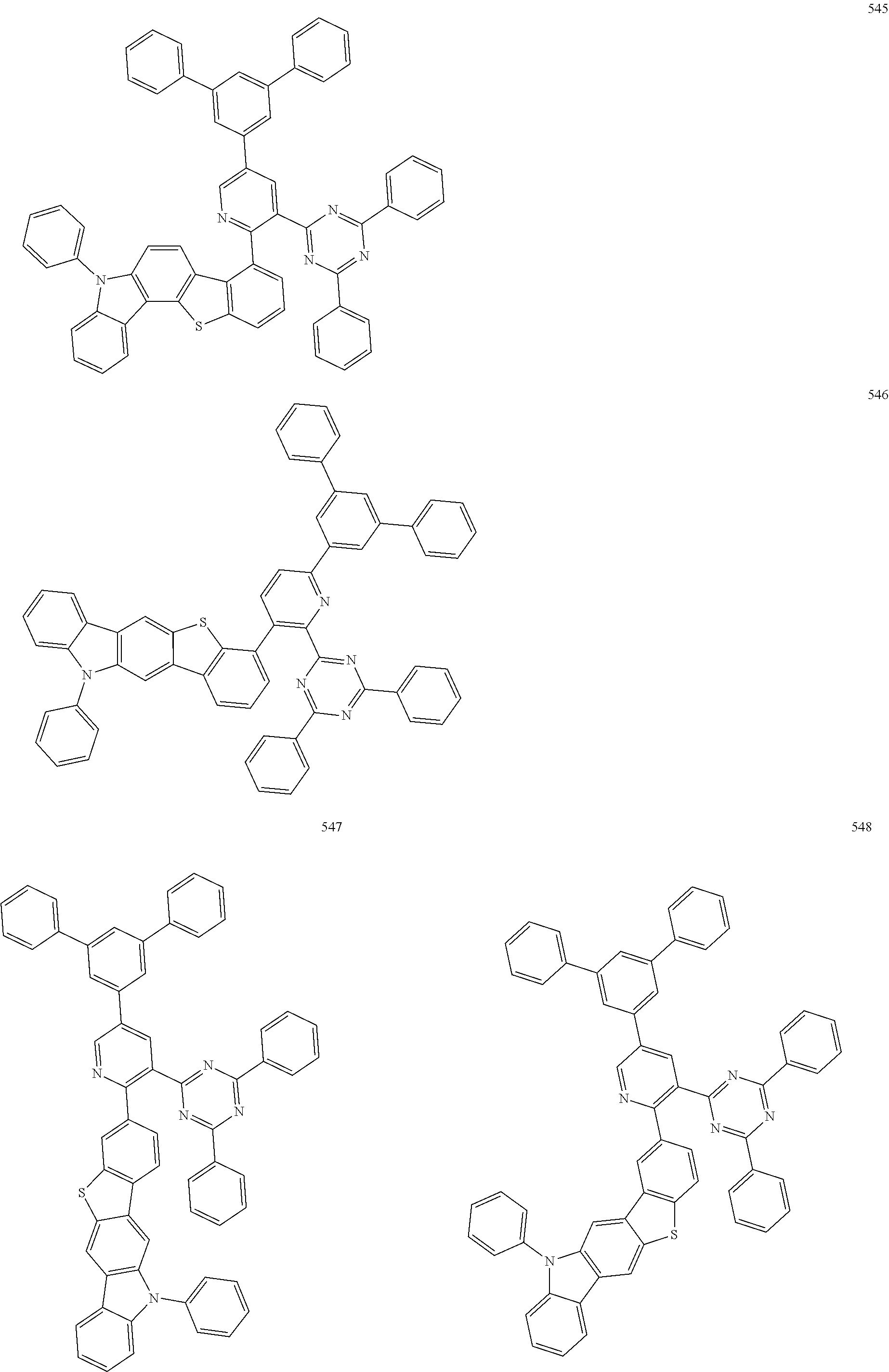

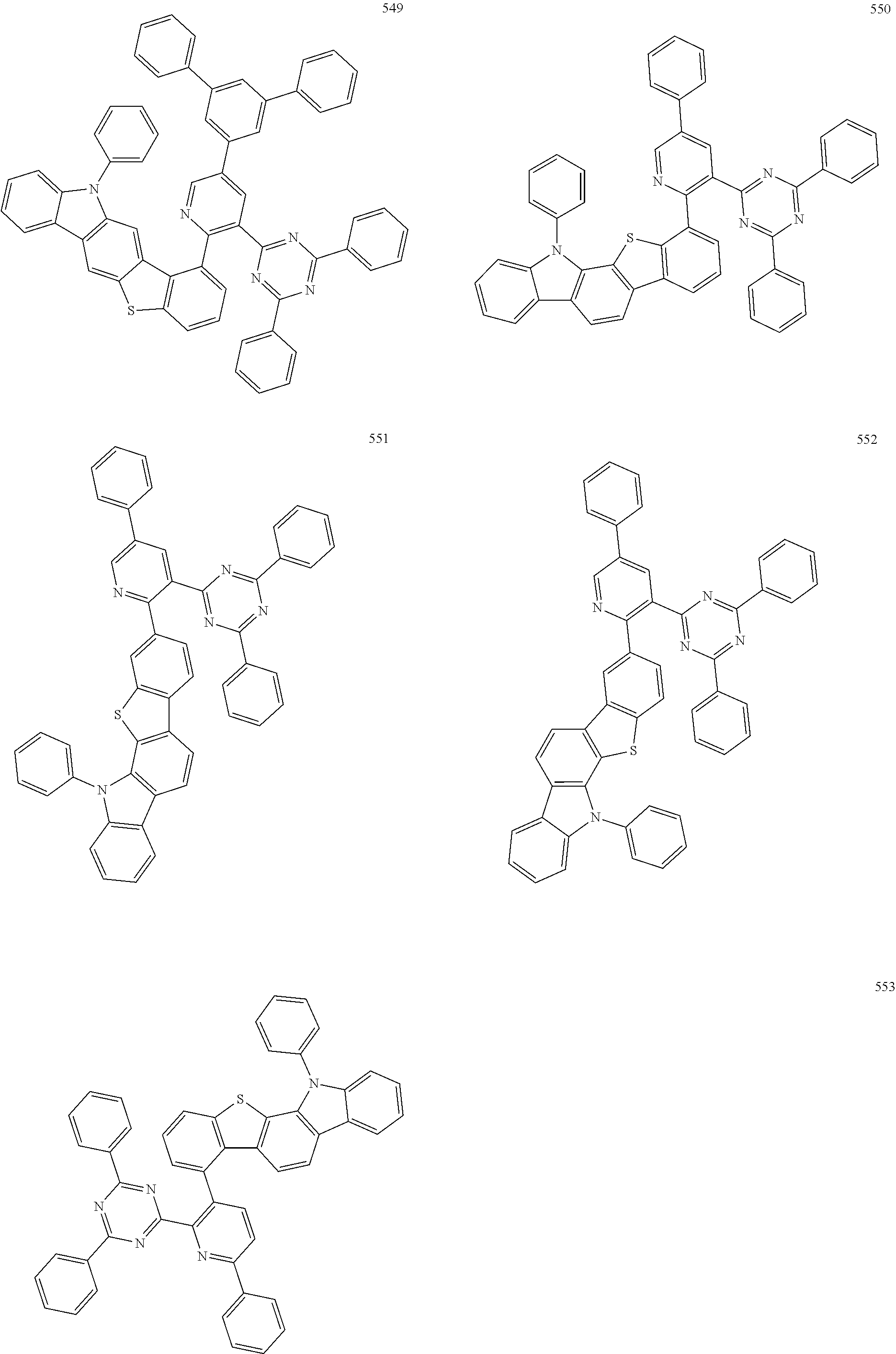

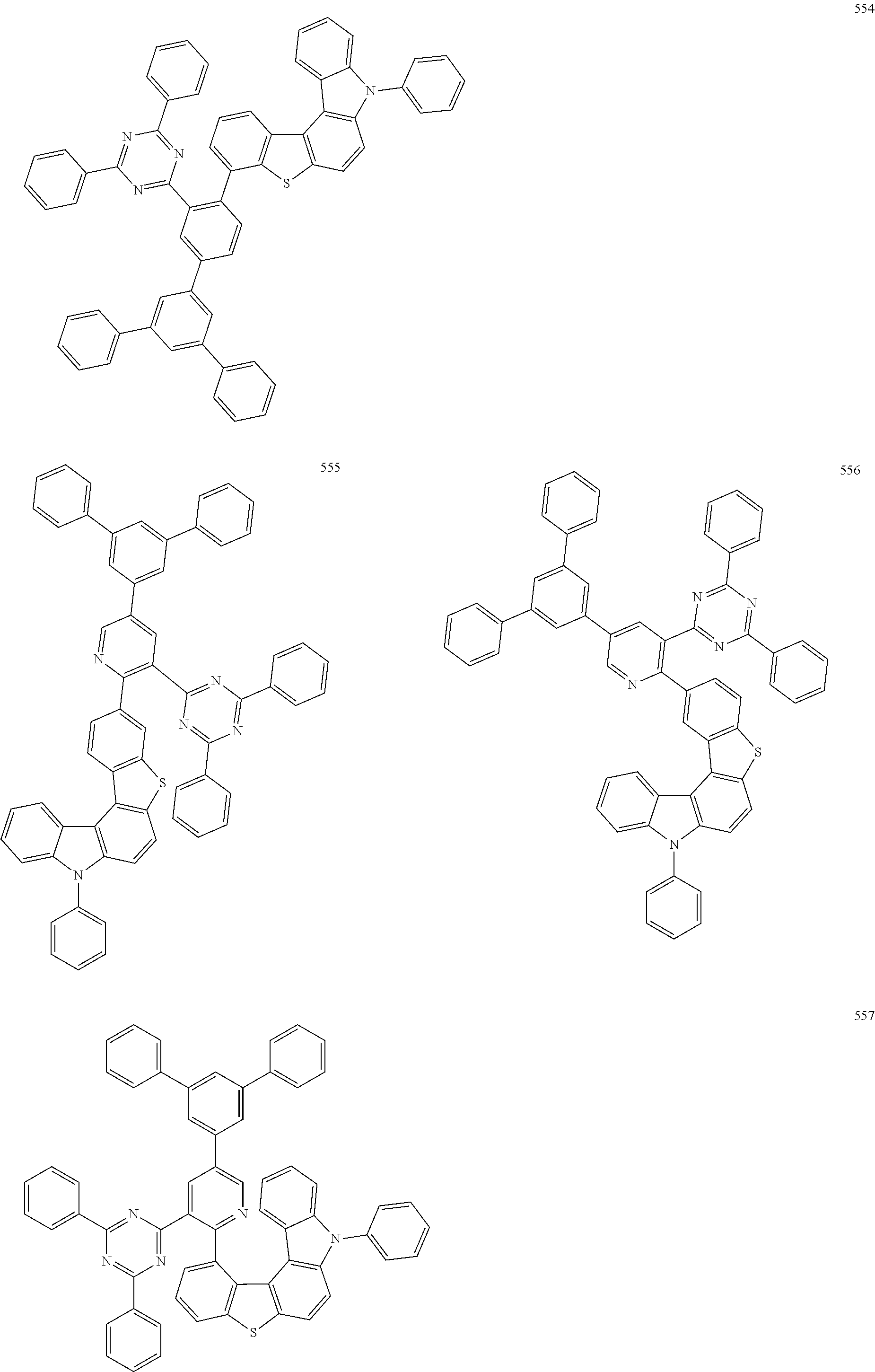 [SMILES: c1ccc(-c2cc(-c3ccccc3)cc(-c3ccc(-c4cccc5c4sc4ccc6c(c7ccccc7n6-c6ccccc6)c45)c(-c4nc(-c5ccccc5)nc(-c5ccccc5)n4)c3)c2)cc1 ; c1ccc(-c2cc(-c3ccccc3)cc(-c3cnc(-c4ccc5c(c4)sc4ccc6c(c7ccccc7n6-c6ccccc6)c45)c(-c4nc(-c5ccccc5)nc(-c5ccccc5)n4)c3)c2)cc1 ; c1ccc(-c2cc(-c3ccccc3)cc(-c3cnc(-c4ccc5sc6ccc7c(c8ccccc8n7-c7ccccc7)c6c5c4)c(-c4nc(-c5ccccc5)nc(-c5ccccc5)n4)c3)c2)cc1 ; c1ccc(-c2cc(-c3ccccc3)cc(-c3cnc(-c4cccc5sc6ccc7c(c8ccccc8n7-c7ccccc7)c6c45)c(-c4nc(-c5ccccc5)nc(-c5ccccc5)n4)c3)c2)cc1]

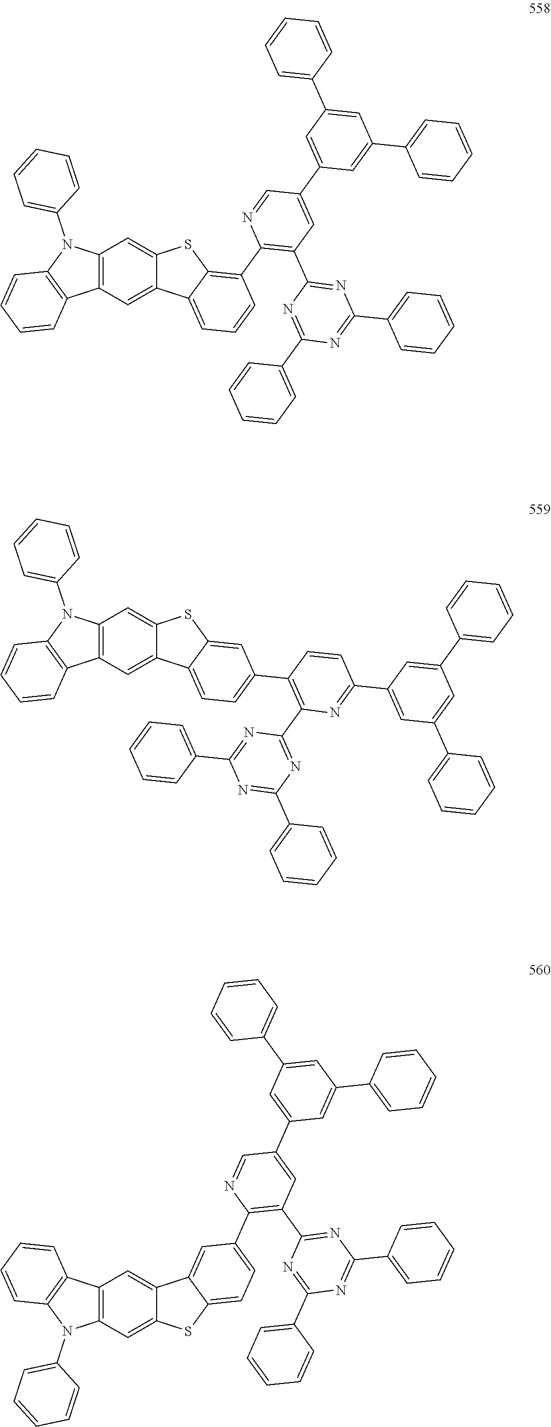 [SMILES: c1ccc(-c2cc(-c3ccccc3)cc(-c3cnc(-c4cccc5c4sc4cc6c(cc45)c4ccccc4n6-c4ccccc4)c(-c4nc(-c5ccccc5)nc(-c5ccccc5)n4)c3)c2)cc1 ; c1ccc(-c2cc(-c3ccccc3)cc(-c3ccc(-c4ccc5c(c4)sc4cc6c(cc45)c4ccccc4n6-c4ccccc4)c(-c4nc(-c5ccccc5)nc(-c5ccccc5)n4)n3)c2)cc1 ; c1ccc(-c2cc(-c3ccccc3)cc(-c3cnc(-c4ccc5sc6cc7c(cc6c5c4)c4ccccc4n7-c4ccccc4)c(-c4nc(-c5ccccc5)nc(-c5ccccc5)n4)c3)c2)cc1]

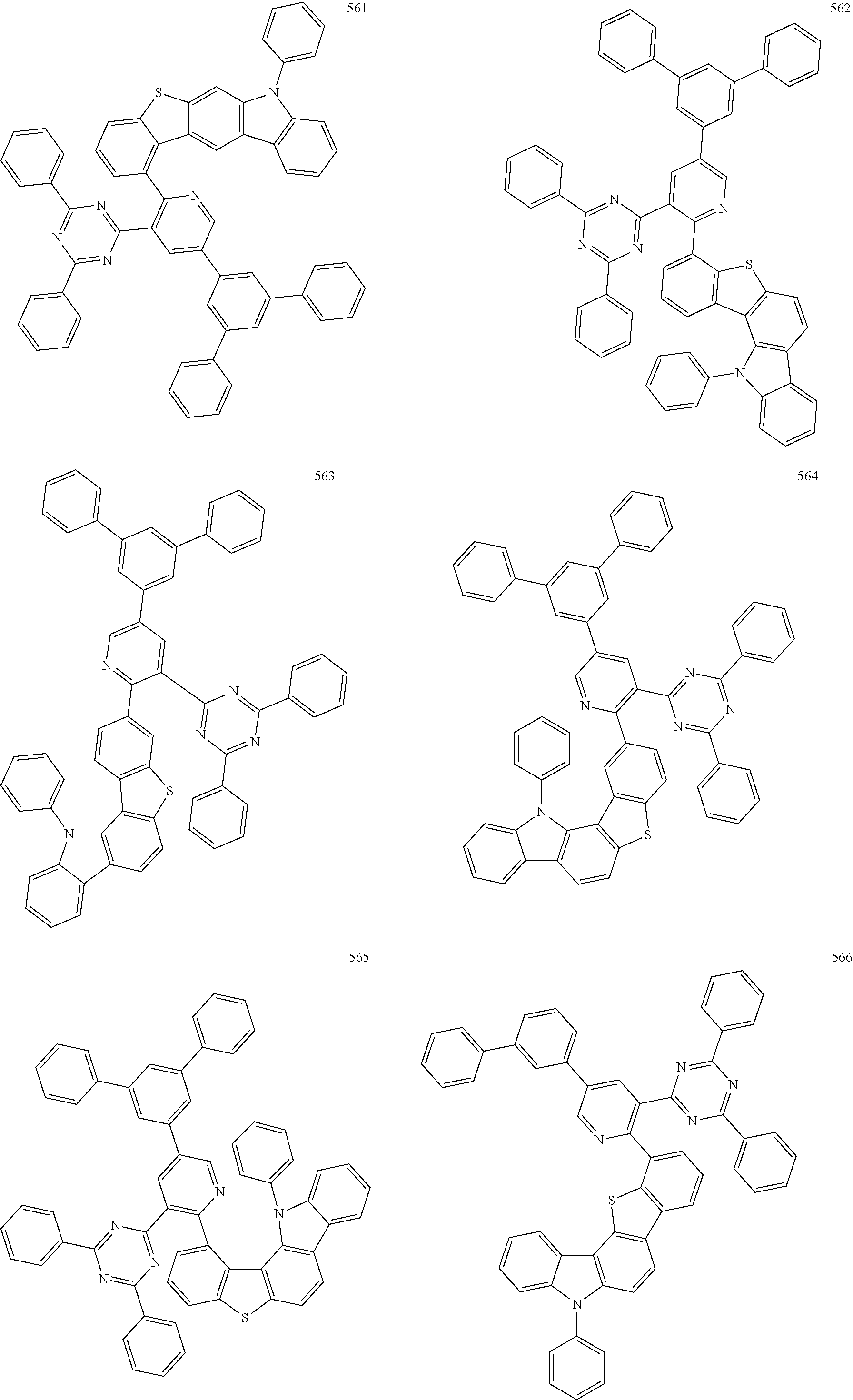

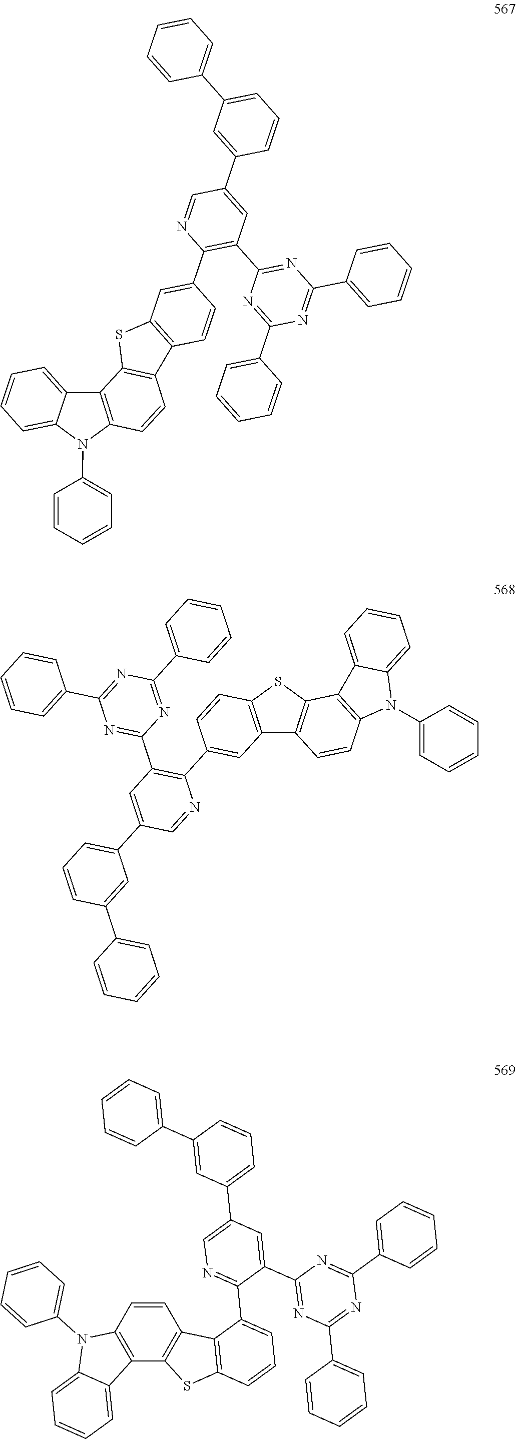 [SMILES: c1ccc(-c2cccc(-c3cnc(-c4ccc5c(c4)sc4c5ccc5c4c4ccccc4n5-c4ccccc4)c(-c4nc(-c5ccccc5)nc(-c5ccccc5)n4)c3)c2)cc1 ; c1ccc(-c2cccc(-c3cnc(-c4ccc5sc6c(ccc7c6c6ccccc6n7-c6ccccc6)c5c4)c(-c4nc(-c5ccccc5)nc(-c5ccccc5)n4)c3)c2)cc1 ; c1ccc(-c2cccc(-c3cnc(-c4cccc5sc6c(ccc7c6c6ccccc6n7-c6ccccc6)c45)c(-c4nc(-c5ccccc5)nc(-c5ccccc5)n4)c3)c2)cc1]

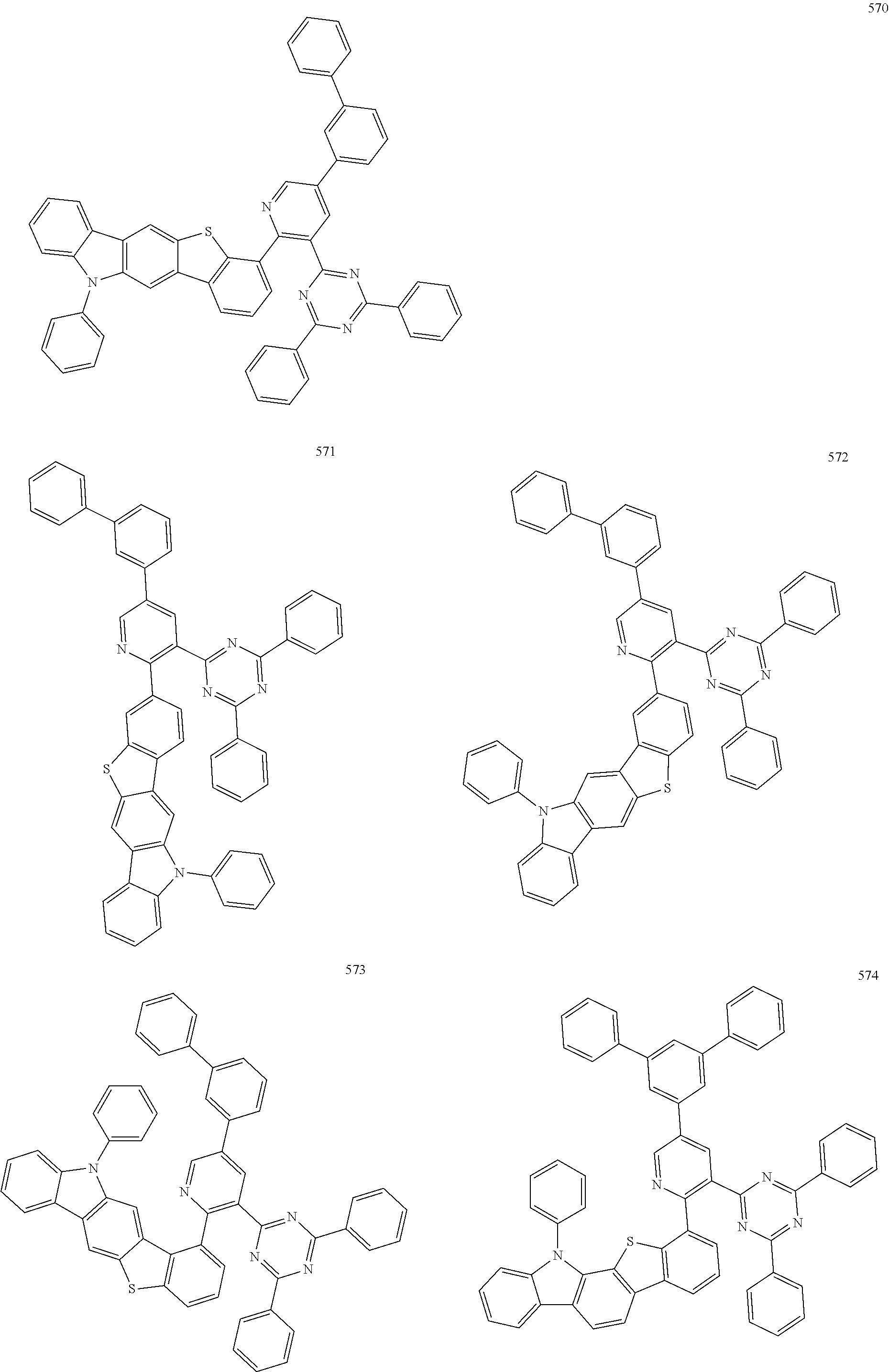

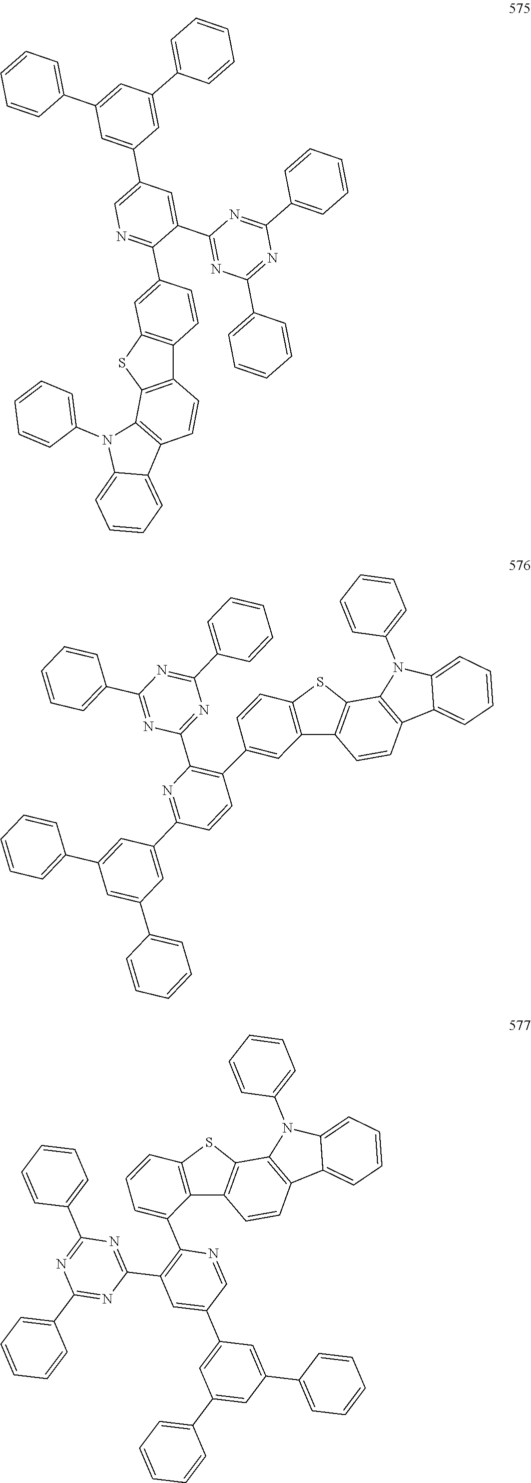 [SMILES: c1ccc(-c2cc(-c3ccccc3)cc(-c3cnc(-c4ccc5c(c4)sc4c5ccc5c6ccccc6n(-c6ccccc6)c54)c(-c4nc(-c5ccccc5)nc(-c5ccccc5)n4)c3)c2)cc1 ; c1ccc(-c2cc(-c3ccccc3)cc(-c3ccc(-c4ccc5sc6c(ccc7c8ccccc8n(-c8ccccc8)c76)c5c4)c(-c4nc(-c5ccccc5)nc(-c5ccccc5)n4)n3)c2)cc1 ; c1ccc(-c2cc(-c3ccccc3)cc(-c3cnc(-c4cccc5sc6c(ccc7c8ccccc8n(-c8ccccc8)c76)c45)c(-c4nc(-c5ccccc5)nc(-c5ccccc5)n4)c3)c2)cc1]

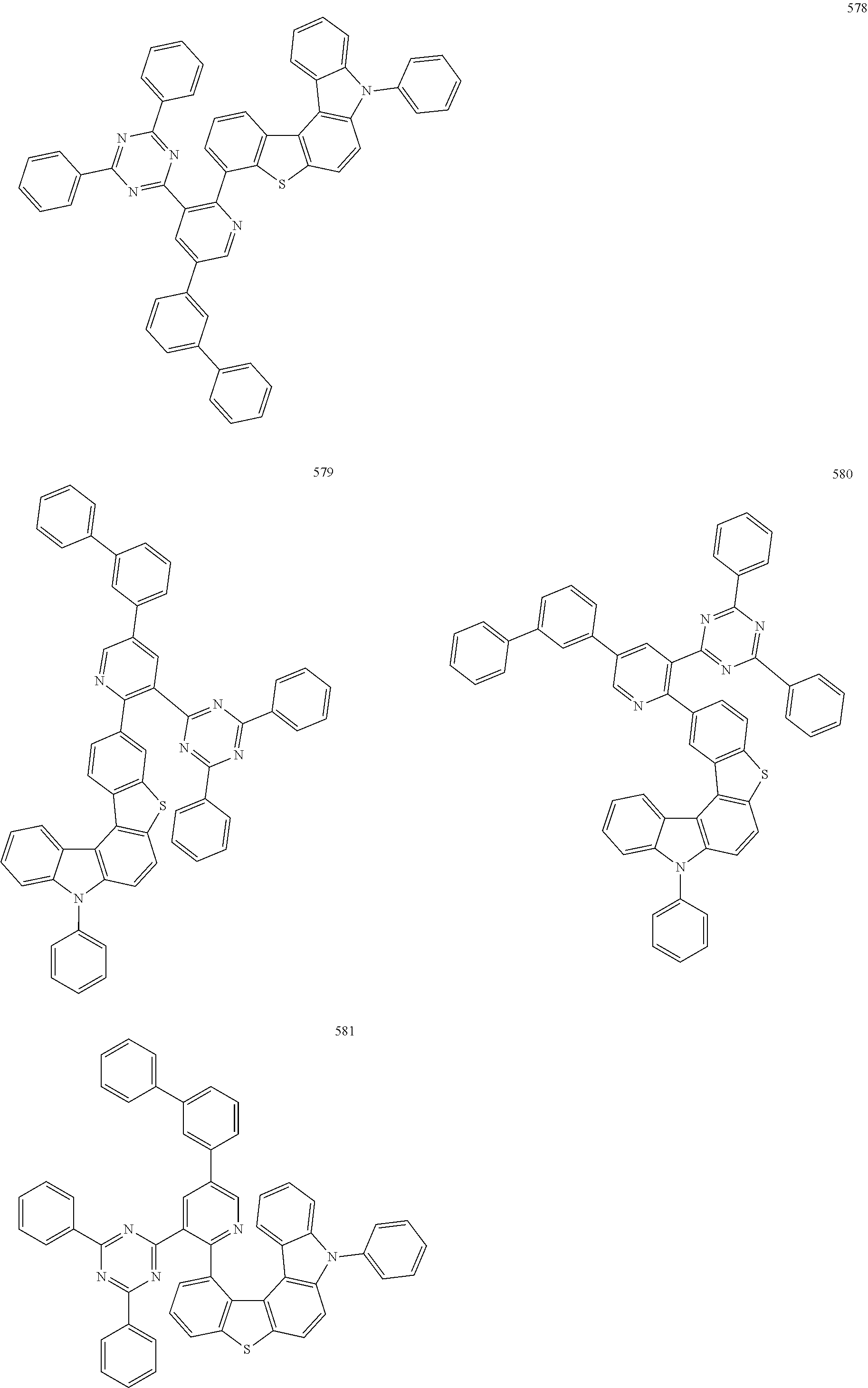 [SMILES: c1ccc(-c2cccc(-c3cnc(-c4cccc5c4sc4ccc6c(c7ccccc7n6-c6ccccc6)c45)c(-c4nc(-c5ccccc5)nc(-c5ccccc5)n4)c3)c2)cc1 ; c1ccc(-c2cccc(-c3cnc(-c4ccc5c(c4)sc4ccc6c(c7ccccc7n6-c6ccccc6)c45)c(-c4nc(-c5ccccc5)nc(-c5ccccc5)n4)c3)c2)cc1 ; c1ccc(-c2cccc(-c3cnc(-c4ccc5sc6ccc7c(c8ccccc8n7-c7ccccc7)c6c5c4)c(-c4nc(-c5ccccc5)nc(-c5ccccc5)n4)c3)c2)cc1 ; c1ccc(-c2cccc(-c3cnc(-c4cccc5sc6ccc7c(c8ccccc8n7-c7ccccc7)c6c45)c(-c4nc(-c5ccccc5)nc(-c5ccccc5)n4)c3)c2)cc1]

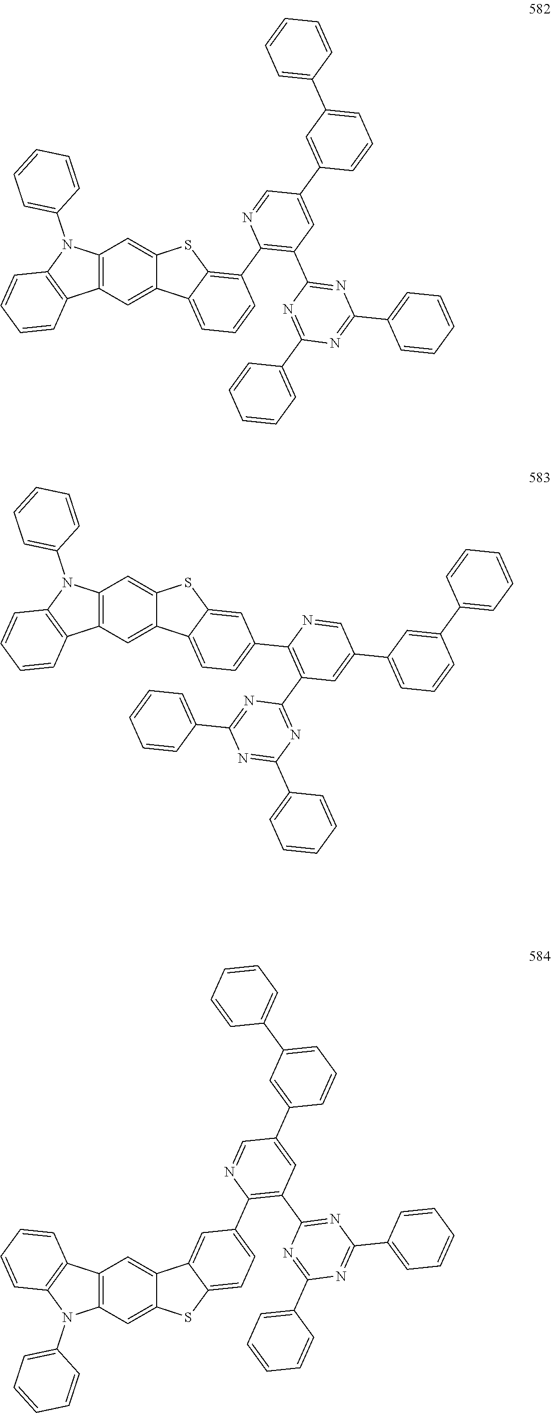 [SMILES: c1ccc(-c2cccc(-c3cnc(-c4cccc5c4sc4cc6c(cc45)c4ccccc4n6-c4ccccc4)c(-c4nc(-c5ccccc5)nc(-c5ccccc5)n4)c3)c2)cc1 ; c1ccc(-c2cccc(-c3cnc(-c4ccc5c(c4)sc4cc6c(cc45)c4ccccc4n6-c4ccccc4)c(-c4nc(-c5ccccc5)nc(-c5ccccc5)n4)c3)c2)cc1 ; c1ccc(-c2cccc(-c3cnc(-c4ccc5sc6cc7c(cc6c5c4)c4ccccc4n7-c4ccccc4)c(-c4nc(-c5ccccc5)nc(-c5ccccc5)n4)c3)c2)cc1]

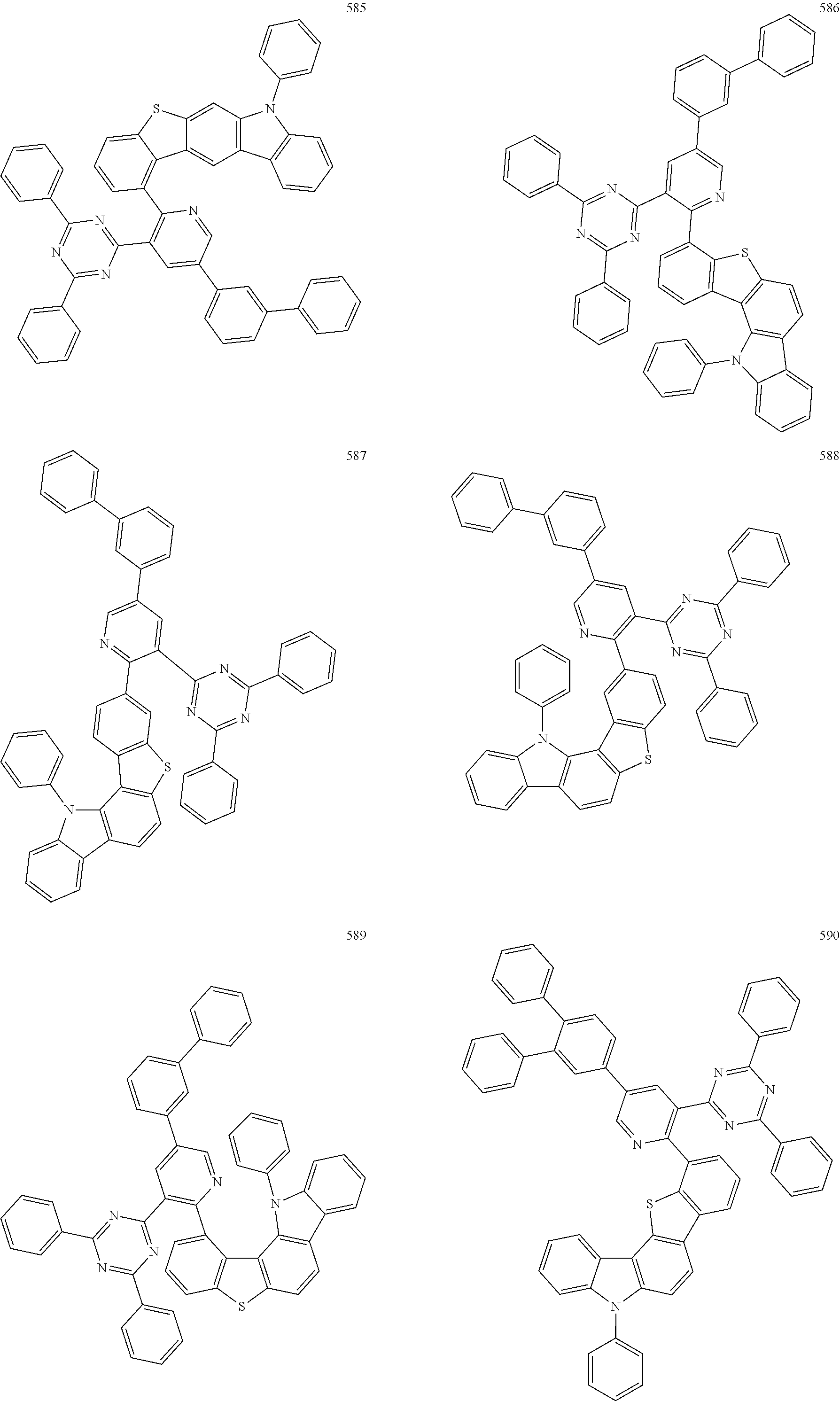

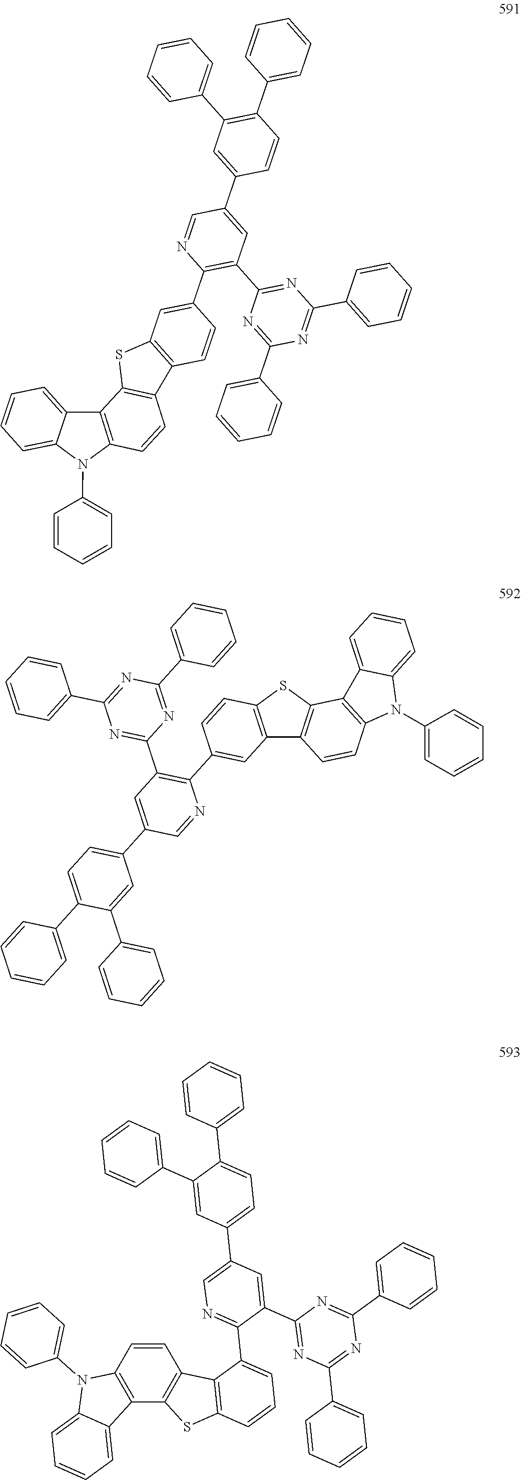 [SMILES: c1ccc(-c2nc(-c3ccccc3)nc(-c3cc(-c4ccc(-c5ccccc5)c(-c5ccccc5)c4)cnc3-c3ccc4c(c3)sc3c4ccc4c3c3ccccc3n4-c3ccccc3)n2)cc1 ; c1ccc(-c2nc(-c3ccccc3)nc(-c3cc(-c4ccc(-c5ccccc5)c(-c5ccccc5)c4)cnc3-c3ccc4sc5c(ccc6c5c5ccccc5n6-c5ccccc5)c4c3)n2)cc1 ; c1ccc(-c2nc(-c3ccccc3)nc(-c3cc(-c4ccc(-c5ccccc5)c(-c5ccccc5)c4)cnc3-c3cccc4sc5c(ccc6c5c5ccccc5n6-c5ccccc5)c34)n2)cc1]

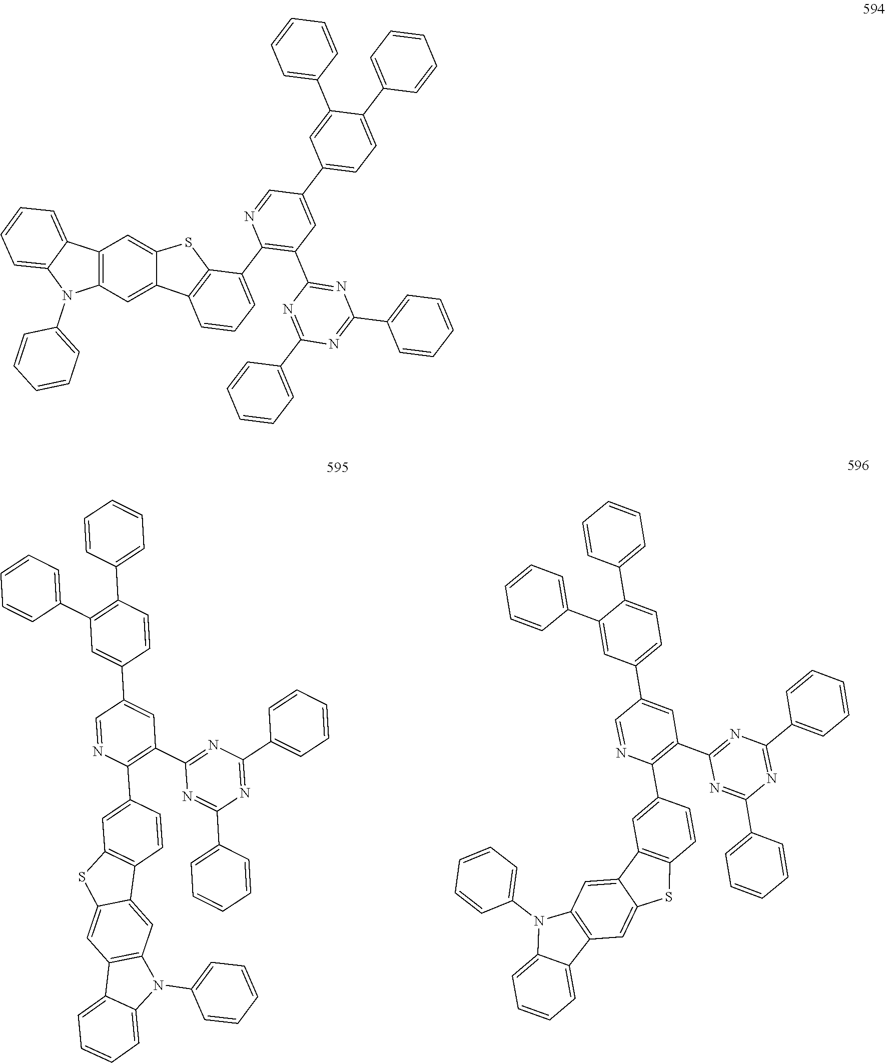 [SMILES: c1ccc(-c2nc(-c3ccccc3)nc(-c3cc(-c4ccc(-c5ccccc5)c(-c5ccccc5)c4)cnc3-c3cccc4c3sc3cc5c6ccccc6n(-c6ccccc6)c5cc34)n2)cc1 ; c1ccc(-c2nc(-c3ccccc3)nc(-c3cc(-c4ccc(-c5ccccc5)c(-c5ccccc5)c4)cnc3-c3ccc4c(c3)sc3cc5c6ccccc6n(-c6ccccc6)c5cc34)n2)cc1 ; c1ccc(-c2nc(-c3ccccc3)nc(-c3cc(-c4ccc(-c5ccccc5)c(-c5ccccc5)c4)cnc3-c3ccc4sc5cc6c7ccccc7n(-c7ccccc7)c6cc5c4c3)n2)cc1]

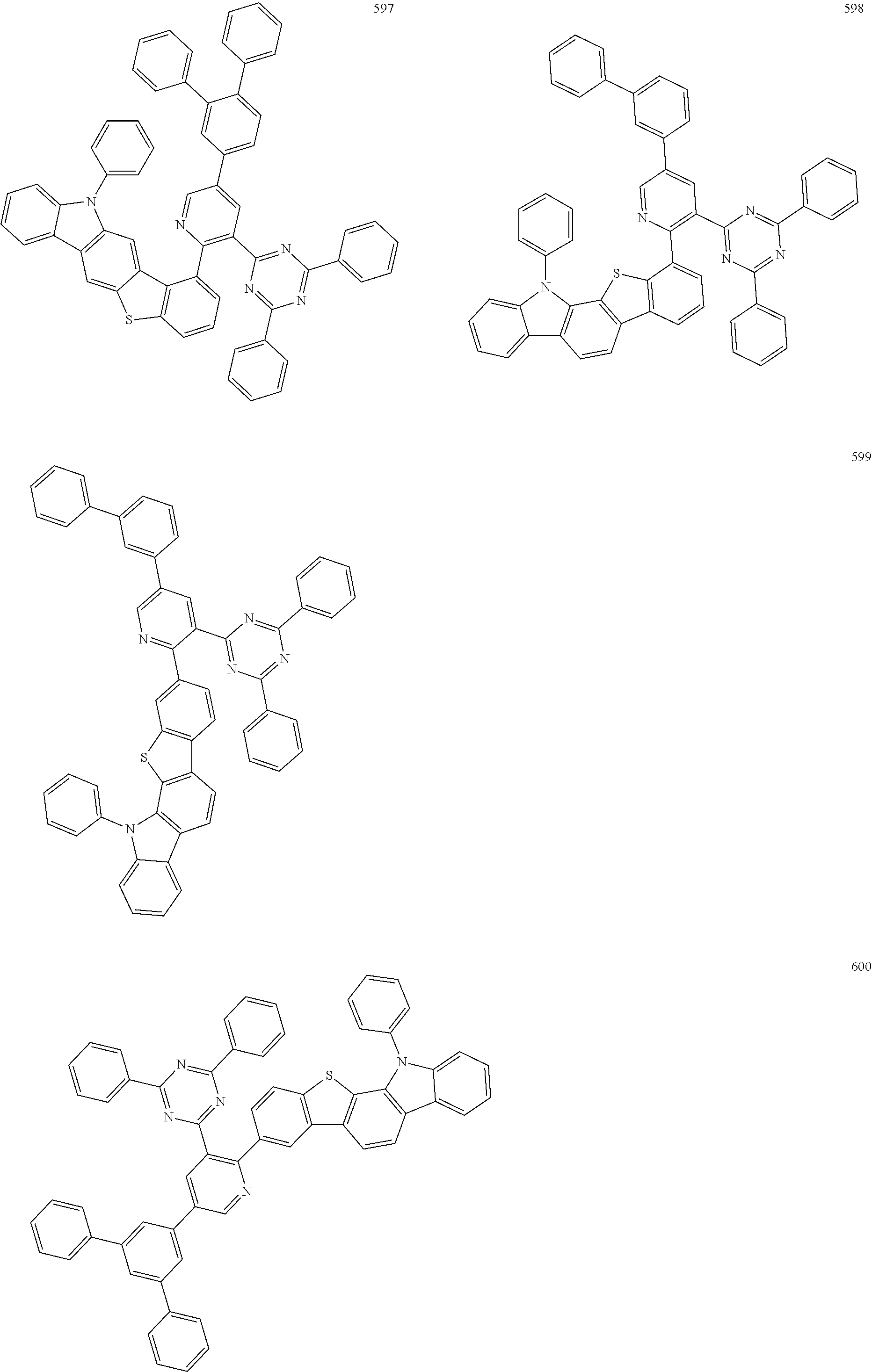 [SMILES: c1ccc(-c2nc(-c3ccccc3)nc(-c3cc(-c4ccc(-c5ccccc5)c(-c5ccccc5)c4)cnc3-c3cccc4sc5cc6c7ccccc7n(-c7ccccc7)c6cc5c34)n2)cc1 ; c1ccc(-c2cccc(-c3cnc(-c4cccc5c4sc4c5ccc5c6ccccc6n(-c6ccccc6)c54)c(-c4nc(-c5ccccc5)nc(-c5ccccc5)n4)c3)c2)cc1 ; c1ccc(-c2cccc(-c3cnc(-c4ccc5c(c4)sc4c5ccc5c6ccccc6n(-c6ccccc6)c54)c(-c4nc(-c5ccccc5)nc(-c5ccccc5)n4)c3)c2)cc1 ; c1ccc(-c2cc(-c3ccccc3)cc(-c3cnc(-c4ccc5sc6c(ccc7c8ccccc8n(-c8ccccc8)c76)c5c4)c(-c4nc(-c5ccccc5)nc(-c5ccccc5)n4)c3)c2)cc1]

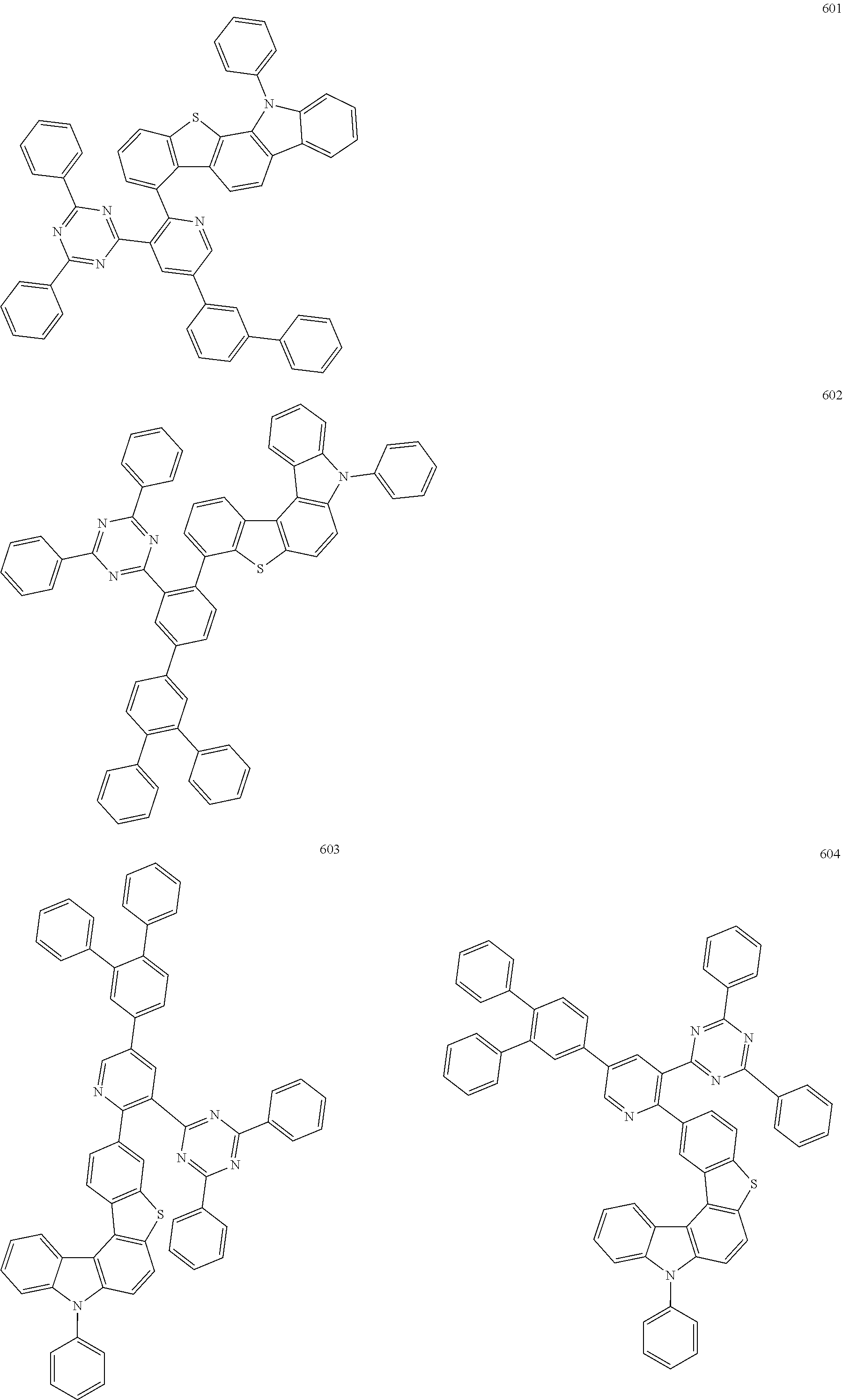

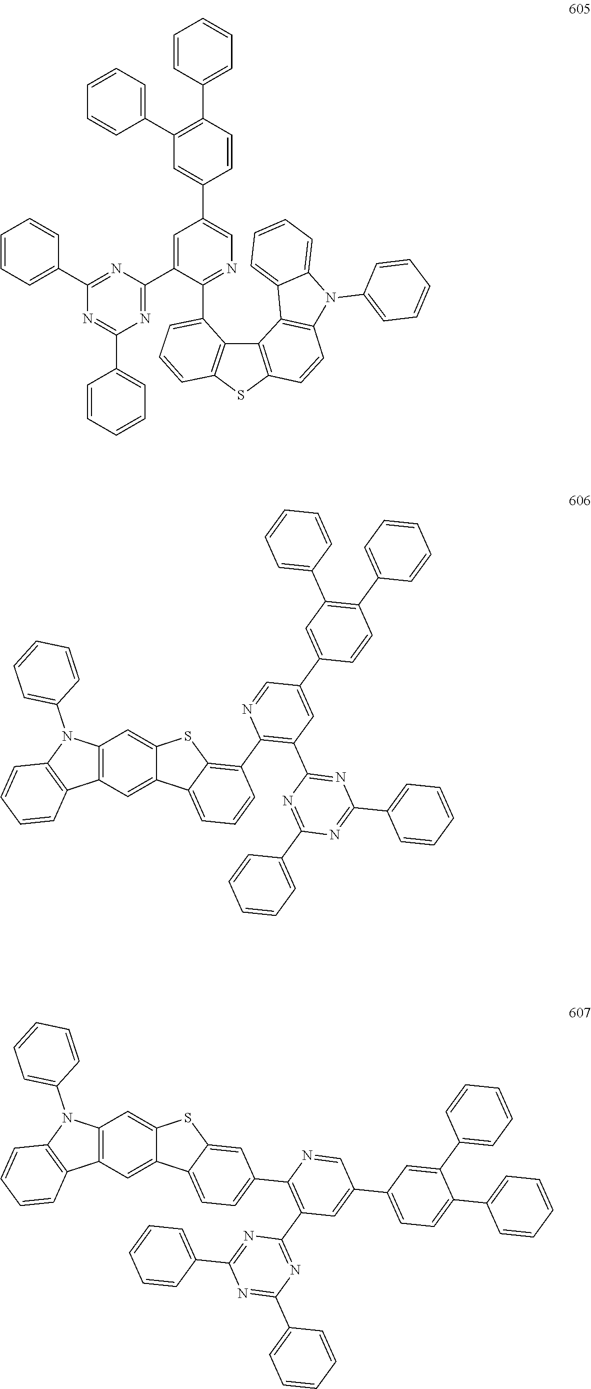 [SMILES: c1ccc(-c2nc(-c3ccccc3)nc(-c3cc(-c4ccc(-c5ccccc5)c(-c5ccccc5)c4)cnc3-c3cccc4sc5ccc6c(c7ccccc7n6-c6ccccc6)c5c34)n2)cc1 ; c1ccc(-c2nc(-c3ccccc3)nc(-c3cc(-c4ccc(-c5ccccc5)c(-c5ccccc5)c4)cnc3-c3cccc4c3sc3cc5c(cc34)c3ccccc3n5-c3ccccc3)n2)cc1 ; c1ccc(-c2nc(-c3ccccc3)nc(-c3cc(-c4ccc(-c5ccccc5)c(-c5ccccc5)c4)cnc3-c3ccc4c(c3)sc3cc5c(cc34)c3ccccc3n5-c3ccccc3)n2)cc1]

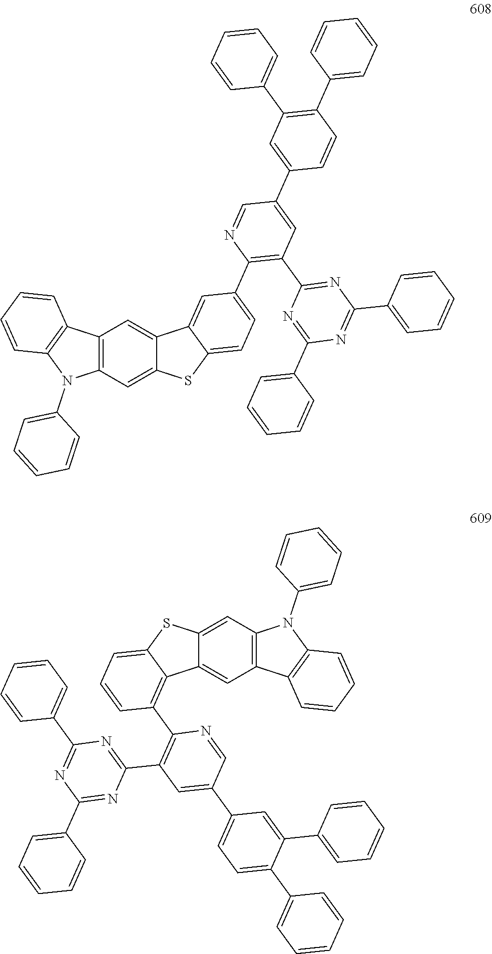 [SMILES: c1ccc(-c2nc(-c3ccccc3)nc(-c3cc(-c4ccc(-c5ccccc5)c(-c5ccccc5)c4)cnc3-c3ccc4sc5cc6c(cc5c4c3)c3ccccc3n6-c3ccccc3)n2)cc1 ; c1ccc(-c2nc(-c3ccccc3)nc(-c3cc(-c4ccc(-c5ccccc5)c(-c5ccccc5)c4)cnc3-c3cccc4sc5cc6c(cc5c34)c3ccccc3n6-c3ccccc3)n2)cc1]

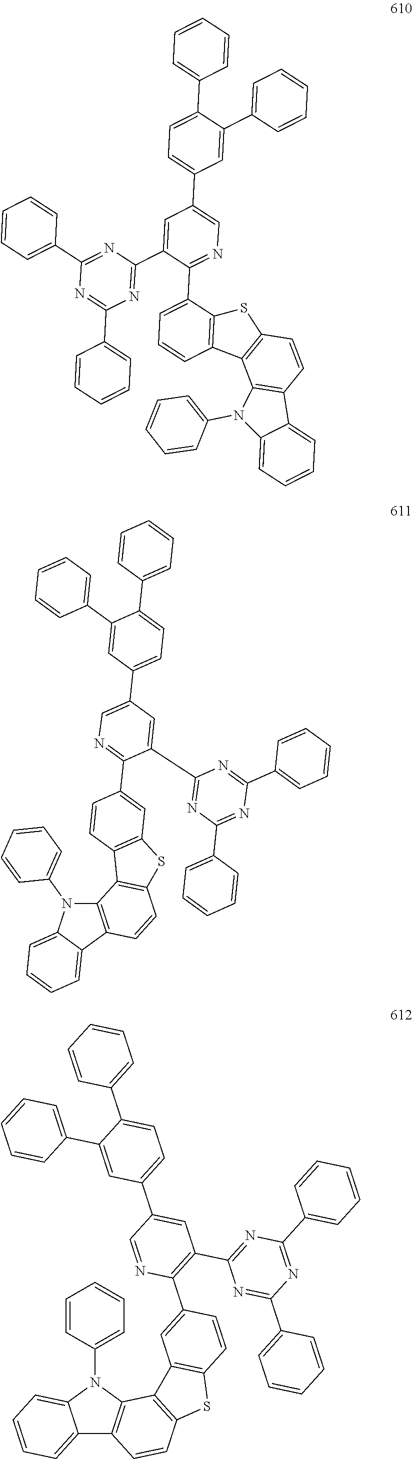 [SMILES: c1ccc(-c2nc(-c3ccccc3)nc(-c3cc(-c4ccc(-c5ccccc5)c(-c5ccccc5)c4)cnc3-c3cccc4c3sc3ccc5c6ccccc6n(-c6ccccc6)c5c34)n2)cc1 ; c1ccc(-c2nc(-c3ccccc3)nc(-c3cc(-c4ccc(-c5ccccc5)c(-c5ccccc5)c4)cnc3-c3ccc4c(c3)sc3ccc5c6ccccc6n(-c6ccccc6)c5c34)n2)cc1 ; c1ccc(-c2nc(-c3ccccc3)nc(-c3cc(-c4ccc(-c5ccccc5)c(-c5ccccc5)c4)cnc3-c3ccc4sc5ccc6c7ccccc7n(-c7ccccc7)c6c5c4c3)n2)cc1]

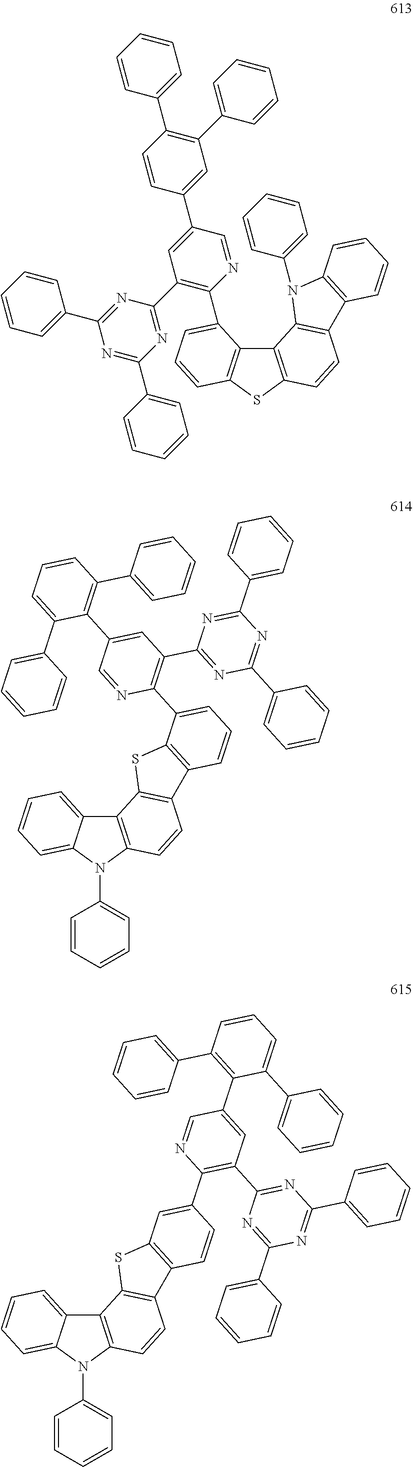 [SMILES: c1ccc(-c2nc(-c3ccccc3)nc(-c3cc(-c4ccc(-c5ccccc5)c(-c5ccccc5)c4)cnc3-c3cccc4sc5ccc6c7ccccc7n(-c7ccccc7)c6c5c34)n2)cc1 ; c1ccc(-c2nc(-c3ccccc3)nc(-c3cc(-c4c(-c5ccccc5)cccc4-c4ccccc4)cnc3-c3cccc4c3sc3c4ccc4c3c3ccccc3n4-c3ccccc3)n2)cc1 ; c1ccc(-c2nc(-c3ccccc3)nc(-c3cc(-c4c(-c5ccccc5)cccc4-c4ccccc4)cnc3-c3ccc4c(c3)sc3c4ccc4c3c3ccccc3n4-c3ccccc3)n2)cc1]

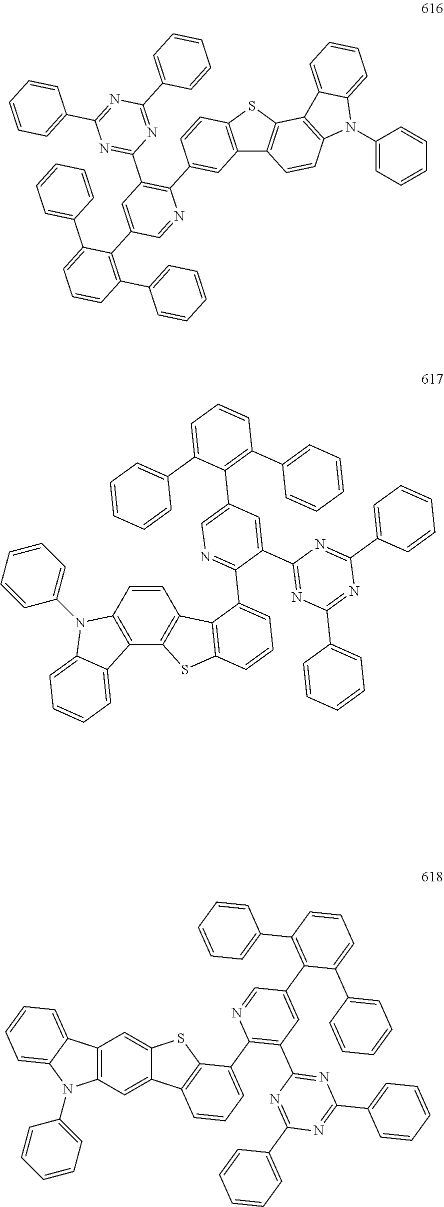 [SMILES: c1ccc(-c2nc(-c3ccccc3)nc(-c3cc(-c4c(-c5ccccc5)cccc4-c4ccccc4)cnc3-c3ccc4sc5c(ccc6c5c5ccccc5n6-c5ccccc5)c4c3)n2)cc1 ; c1ccc(-c2nc(-c3ccccc3)nc(-c3cc(-c4c(-c5ccccc5)cccc4-c4ccccc4)cnc3-c3cccc4sc5c(ccc6c5c5ccccc5n6-c5ccccc5)c34)n2)cc1 ; c1ccc(-c2nc(-c3ccccc3)nc(-c3cc(-c4c(-c5ccccc5)cccc4-c4ccccc4)cnc3-c3cccc4c3sc3cc5c6ccccc6n(-c6ccccc6)c5cc34)n2)cc1]

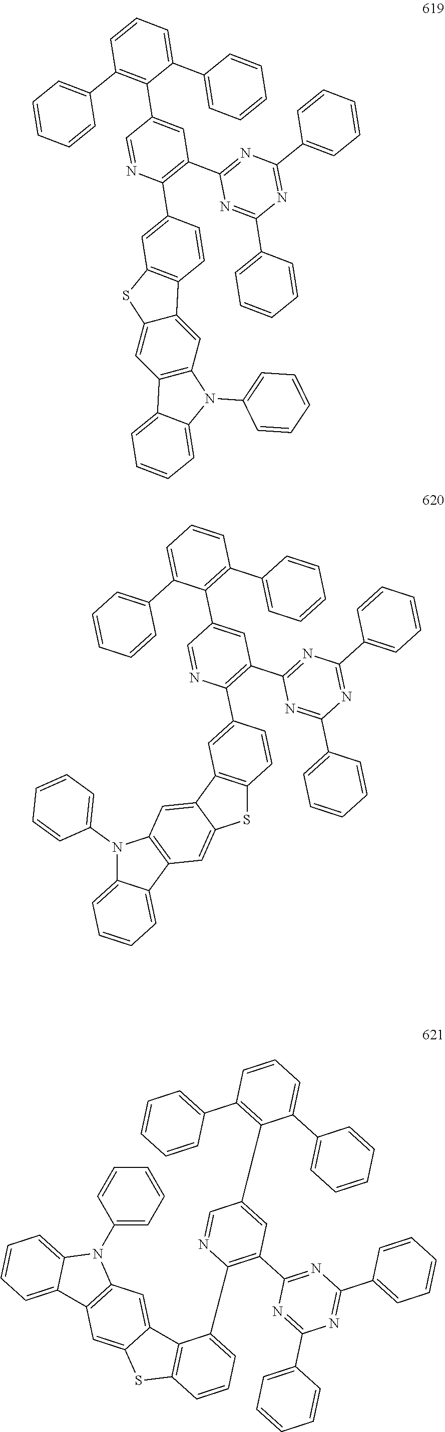 [SMILES: c1ccc(-c2nc(-c3ccccc3)nc(-c3cc(-c4c(-c5ccccc5)cccc4-c4ccccc4)cnc3-c3ccc4c(c3)sc3cc5c6ccccc6n(-c6ccccc6)c5cc34)n2)cc1 ; c1ccc(-c2nc(-c3ccccc3)nc(-c3cc(-c4c(-c5ccccc5)cccc4-c4ccccc4)cnc3-c3ccc4sc5cc6c7ccccc7n(-c7ccccc7)c6cc5c4c3)n2)cc1 ; c1ccc(-c2nc(-c3ccccc3)nc(-c3cc(-c4c(-c5ccccc5)cccc4-c4ccccc4)cnc3-c3cccc4sc5cc6c7ccccc7n(-c7ccccc7)c6cc5c34)n2)cc1]

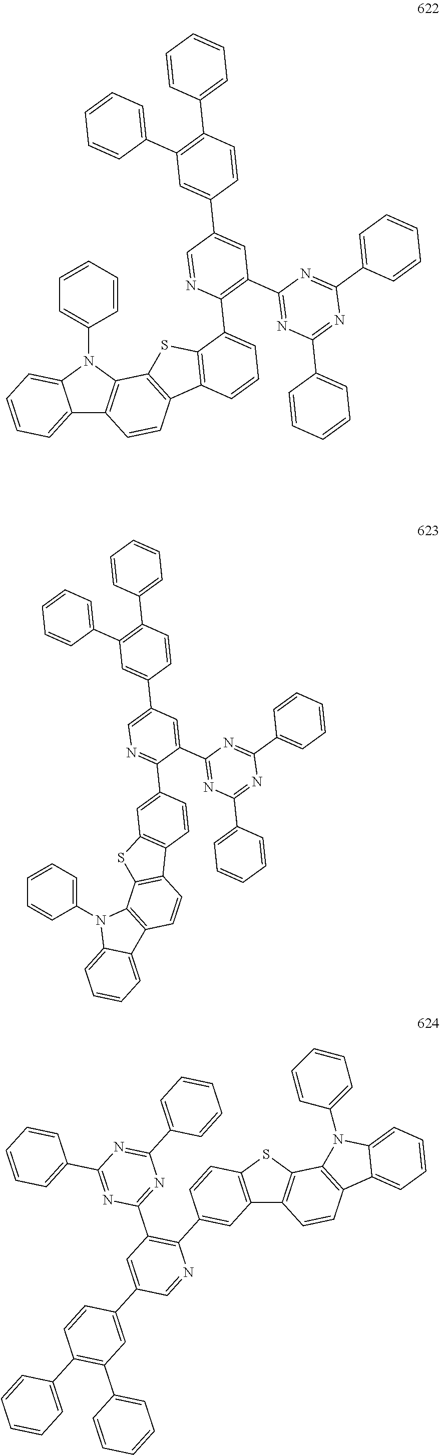 [SMILES: c1ccc(-c2nc(-c3ccccc3)nc(-c3cc(-c4ccc(-c5ccccc5)c(-c5ccccc5)c4)cnc3-c3cccc4c3sc3c4ccc4c5ccccc5n(-c5ccccc5)c43)n2)cc1 ; c1ccc(-c2nc(-c3ccccc3)nc(-c3cc(-c4ccc(-c5ccccc5)c(-c5ccccc5)c4)cnc3-c3ccc4c(c3)sc3c4ccc4c5ccccc5n(-c5ccccc5)c43)n2)cc1 ; c1ccc(-c2nc(-c3ccccc3)nc(-c3cc(-c4ccc(-c5ccccc5)c(-c5ccccc5)c4)cnc3-c3ccc4sc5c(ccc6c7ccccc7n(-c7ccccc7)c65)c4c3)n2)cc1]

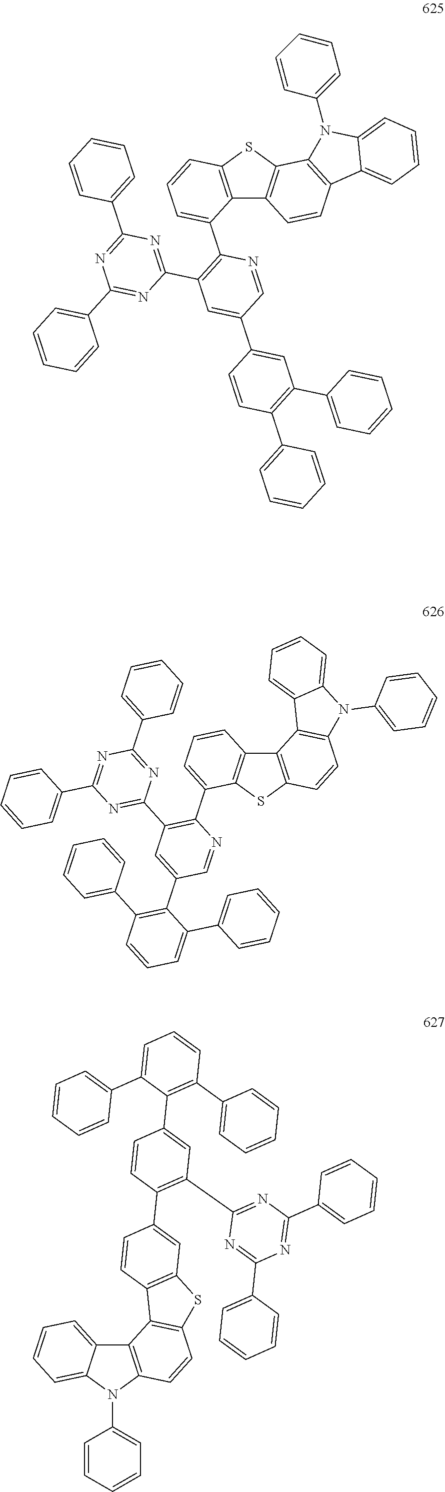 [SMILES: c1ccc(-c2nc(-c3ccccc3)nc(-c3cc(-c4ccc(-c5ccccc5)c(-c5ccccc5)c4)cnc3-c3cccc4sc5c(ccc6c7ccccc7n(-c7ccccc7)c65)c34)n2)cc1 ; c1ccc(-c2nc(-c3ccccc3)nc(-c3cc(-c4c(-c5ccccc5)cccc4-c4ccccc4)cnc3-c3cccc4c3sc3ccc5c(c6ccccc6n5-c5ccccc5)c34)n2)cc1 ; c1ccc(-c2nc(-c3ccccc3)nc(-c3cc(-c4c(-c5ccccc5)cccc4-c4ccccc4)ccc3-c3ccc4c(c3)sc3ccc5c(c6ccccc6n5-c5ccccc5)c34)n2)cc1]

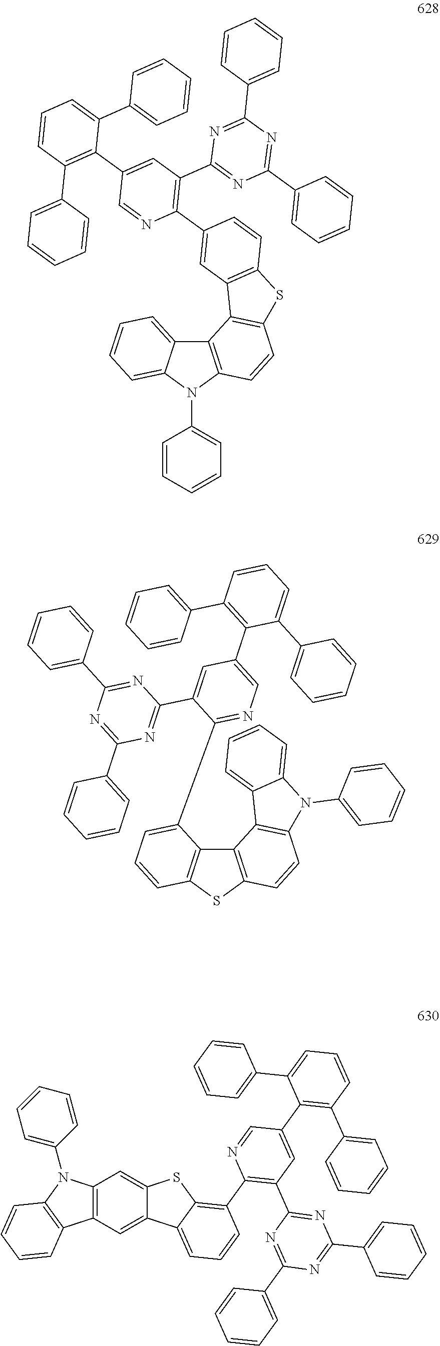 [SMILES: c1ccc(-c2nc(-c3ccccc3)nc(-c3cc(-c4c(-c5ccccc5)cccc4-c4ccccc4)cnc3-c3ccc4sc5ccc6c(c7ccccc7n6-c6ccccc6)c5c4c3)n2)cc1 ; c1ccc(-c2nc(-c3ccccc3)nc(-c3cc(-c4c(-c5ccccc5)cccc4-c4ccccc4)cnc3-c3cccc4sc5ccc6c(c7ccccc7n6-c6ccccc6)c5c34)n2)cc1 ; c1ccc(-c2nc(-c3ccccc3)nc(-c3cc(-c4c(-c5ccccc5)cccc4-c4ccccc4)cnc3-c3cccc4c3sc3cc5c(cc34)c3ccccc3n5-c3ccccc3)n2)cc1]

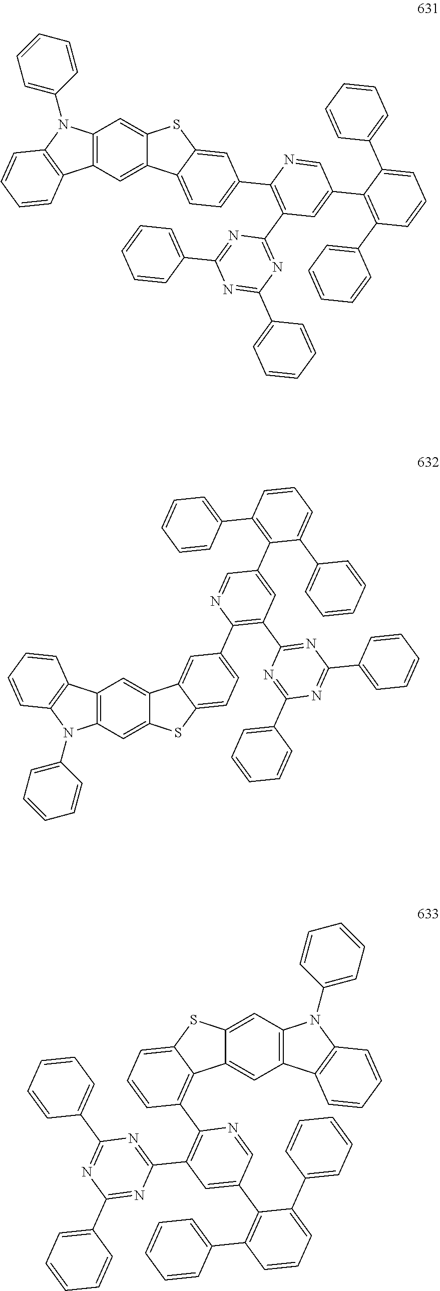 [SMILES: c1ccc(-c2nc(-c3ccccc3)nc(-c3cc(-c4c(-c5ccccc5)cccc4-c4ccccc4)cnc3-c3ccc4c(c3)sc3cc5c(cc34)c3ccccc3n5-c3ccccc3)n2)cc1 ; c1ccc(-c2nc(-c3ccccc3)nc(-c3cc(-c4c(-c5ccccc5)cccc4-c4ccccc4)cnc3-c3ccc4sc5cc6c(cc5c4c3)c3ccccc3n6-c3ccccc3)n2)cc1 ; c1ccc(-c2nc(-c3ccccc3)nc(-c3cc(-c4c(-c5ccccc5)cccc4-c4ccccc4)cnc3-c3cccc4sc5cc6c(cc5c34)c3ccccc3n6-c3ccccc3)n2)cc1]

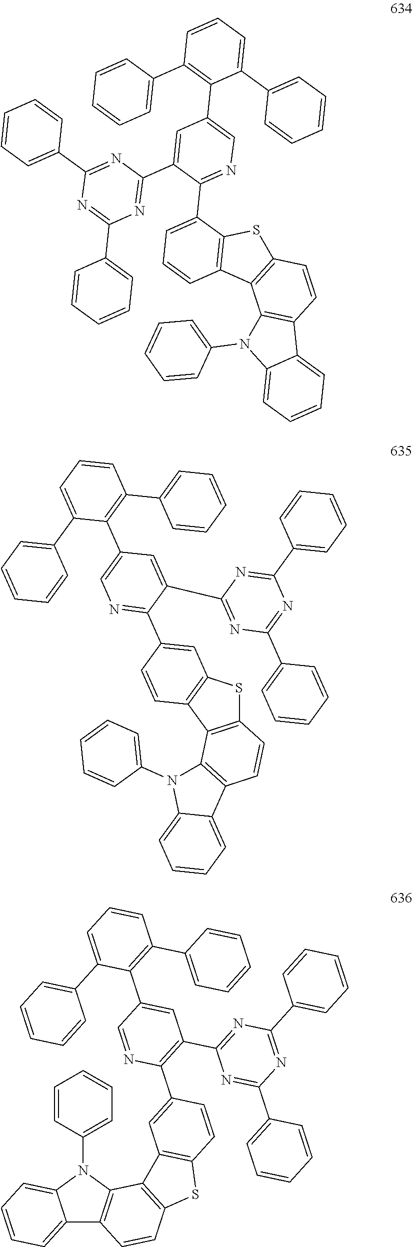 [SMILES: c1ccc(-c2nc(-c3ccccc3)nc(-c3cc(-c4c(-c5ccccc5)cccc4-c4ccccc4)cnc3-c3cccc4c3sc3ccc5c6ccccc6n(-c6ccccc6)c5c34)n2)cc1 ; c1ccc(-c2nc(-c3ccccc3)nc(-c3cc(-c4c(-c5ccccc5)cccc4-c4ccccc4)cnc3-c3ccc4c(c3)sc3ccc5c6ccccc6n(-c6ccccc6)c5c34)n2)cc1 ; c1ccc(-c2nc(-c3ccccc3)nc(-c3cc(-c4c(-c5ccccc5)cccc4-c4ccccc4)cnc3-c3ccc4sc5ccc6c7ccccc7n(-c7ccccc7)c6c5c4c3)n2)cc1]

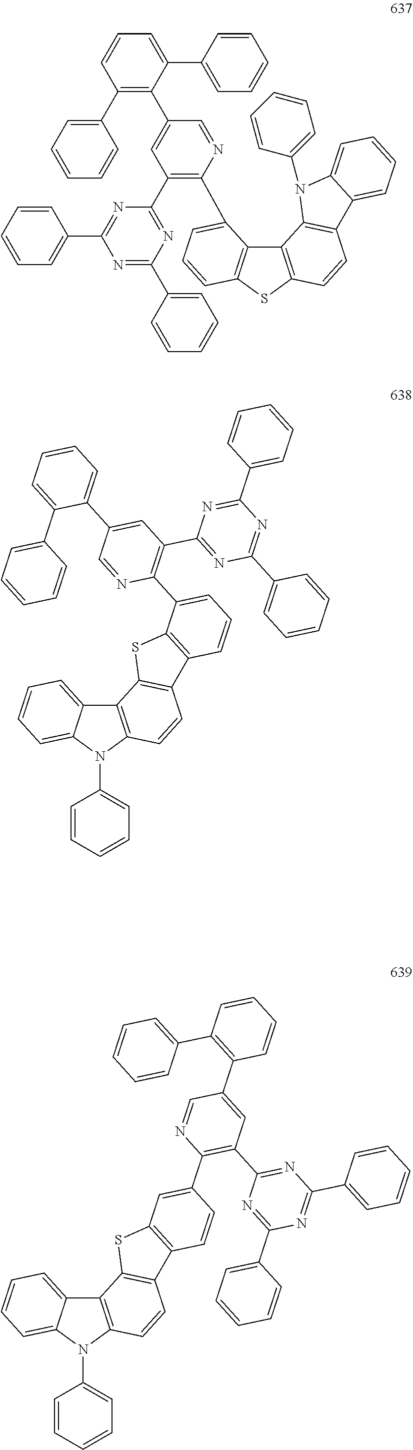 [SMILES: c1ccc(-c2nc(-c3ccccc3)nc(-c3cc(-c4c(-c5ccccc5)cccc4-c4ccccc4)cnc3-c3cccc4sc5ccc6c7ccccc7n(-c7ccccc7)c6c5c34)n2)cc1 ; c1ccc(-c2nc(-c3ccccc3)nc(-c3cc(-c4ccccc4-c4ccccc4)cnc3-c3cccc4c3sc3c4ccc4c3c3ccccc3n4-c3ccccc3)n2)cc1 ; c1ccc(-c2nc(-c3ccccc3)nc(-c3cc(-c4ccccc4-c4ccccc4)cnc3-c3ccc4c(c3)sc3c4ccc4c3c3ccccc3n4-c3ccccc3)n2)cc1]

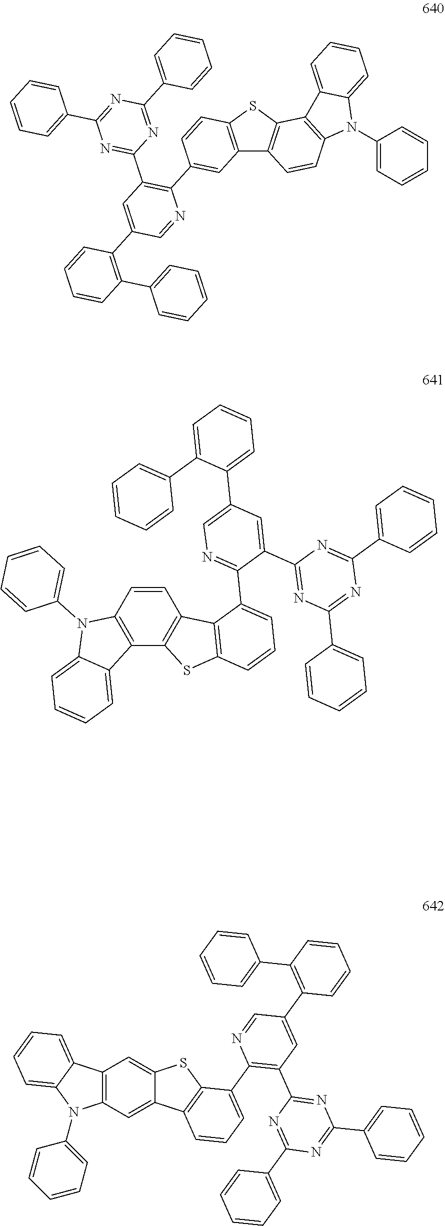 [SMILES: c1ccc(-c2nc(-c3ccccc3)nc(-c3cc(-c4ccccc4-c4ccccc4)cnc3-c3ccc4sc5c(ccc6c5c5ccccc5n6-c5ccccc5)c4c3)n2)cc1 ; c1ccc(-c2nc(-c3ccccc3)nc(-c3cc(-c4ccccc4-c4ccccc4)cnc3-c3cccc4sc5c(ccc6c5c5ccccc5n6-c5ccccc5)c34)n2)cc1 ; c1ccc(-c2nc(-c3ccccc3)nc(-c3cc(-c4ccccc4-c4ccccc4)cnc3-c3cccc4c3sc3cc5c6ccccc6n(-c6ccccc6)c5cc34)n2)cc1]

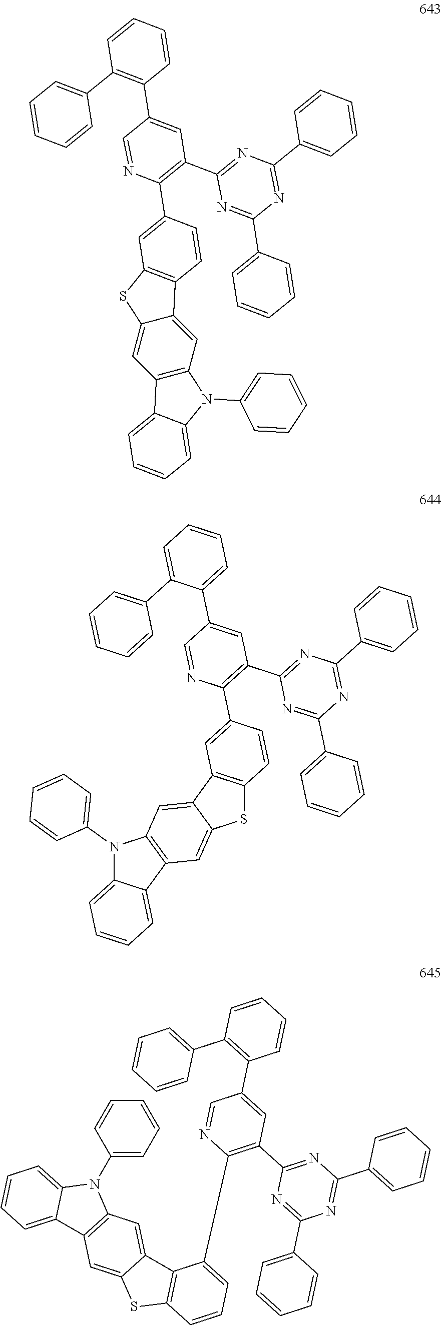 [SMILES: c1ccc(-c2nc(-c3ccccc3)nc(-c3cc(-c4ccccc4-c4ccccc4)cnc3-c3ccc4c(c3)sc3cc5c6ccccc6n(-c6ccccc6)c5cc34)n2)cc1 ; c1ccc(-c2nc(-c3ccccc3)nc(-c3cc(-c4ccccc4-c4ccccc4)cnc3-c3ccc4sc5cc6c7ccccc7n(-c7ccccc7)c6cc5c4c3)n2)cc1 ; c1ccc(-c2nc(-c3ccccc3)nc(-c3cc(-c4ccccc4-c4ccccc4)cnc3-c3cccc4sc5cc6c7ccccc7n(-c7ccccc7)c6cc5c34)n2)cc1]

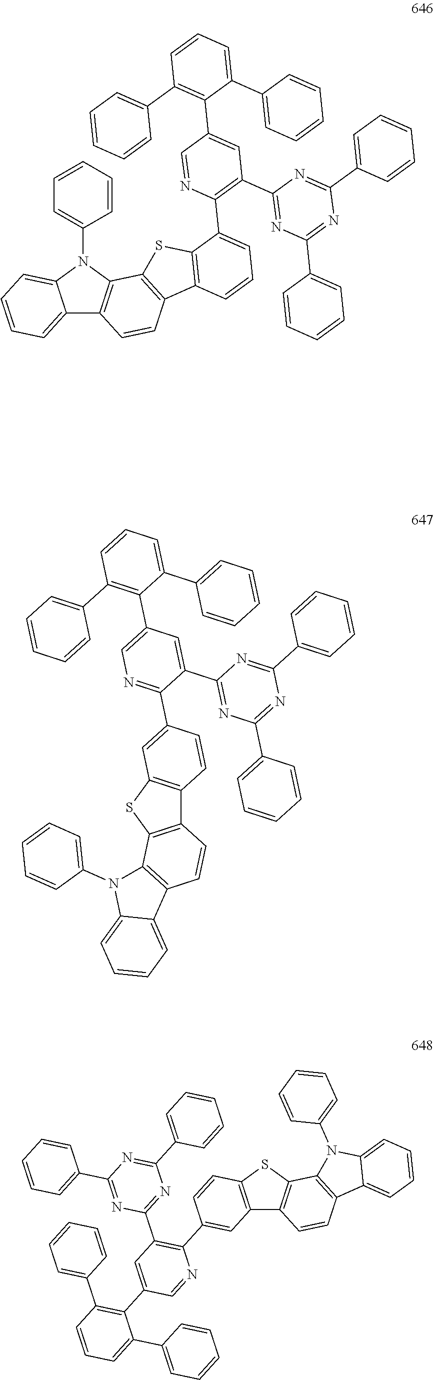 [SMILES: c1ccc(-c2nc(-c3ccccc3)nc(-c3cc(-c4c(-c5ccccc5)cccc4-c4ccccc4)cnc3-c3cccc4c3sc3c4ccc4c5ccccc5n(-c5ccccc5)c43)n2)cc1 ; c1ccc(-c2nc(-c3ccccc3)nc(-c3cc(-c4c(-c5ccccc5)cccc4-c4ccccc4)cnc3-c3ccc4c(c3)sc3c4ccc4c5ccccc5n(-c5ccccc5)c43)n2)cc1 ; c1ccc(-c2nc(-c3ccccc3)nc(-c3cc(-c4c(-c5ccccc5)cccc4-c4ccccc4)cnc3-c3ccc4sc5c(ccc6c7ccccc7n(-c7ccccc7)c65)c4c3)n2)cc1]

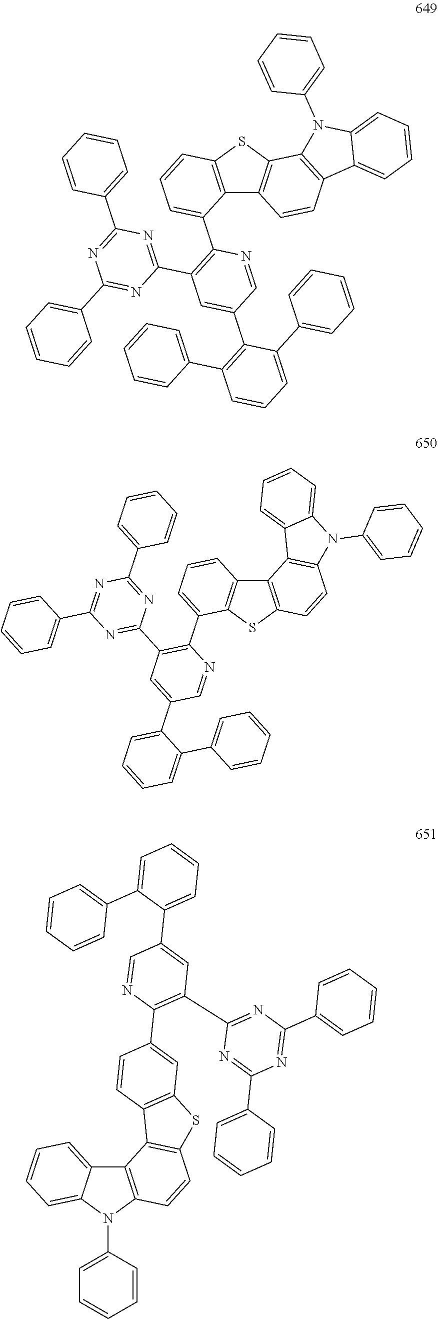 [SMILES: c1ccc(-c2nc(-c3ccccc3)nc(-c3cc(-c4c(-c5ccccc5)cccc4-c4ccccc4)cnc3-c3cccc4sc5c(ccc6c7ccccc7n(-c7ccccc7)c65)c34)n2)cc1 ; c1ccc(-c2nc(-c3ccccc3)nc(-c3cc(-c4ccccc4-c4ccccc4)cnc3-c3cccc4c3sc3ccc5c(c6ccccc6n5-c5ccccc5)c34)n2)cc1 ; c1ccc(-c2nc(-c3ccccc3)nc(-c3cc(-c4ccccc4-c4ccccc4)cnc3-c3ccc4c(c3)sc3ccc5c(c6ccccc6n5-c5ccccc5)c34)n2)cc1]

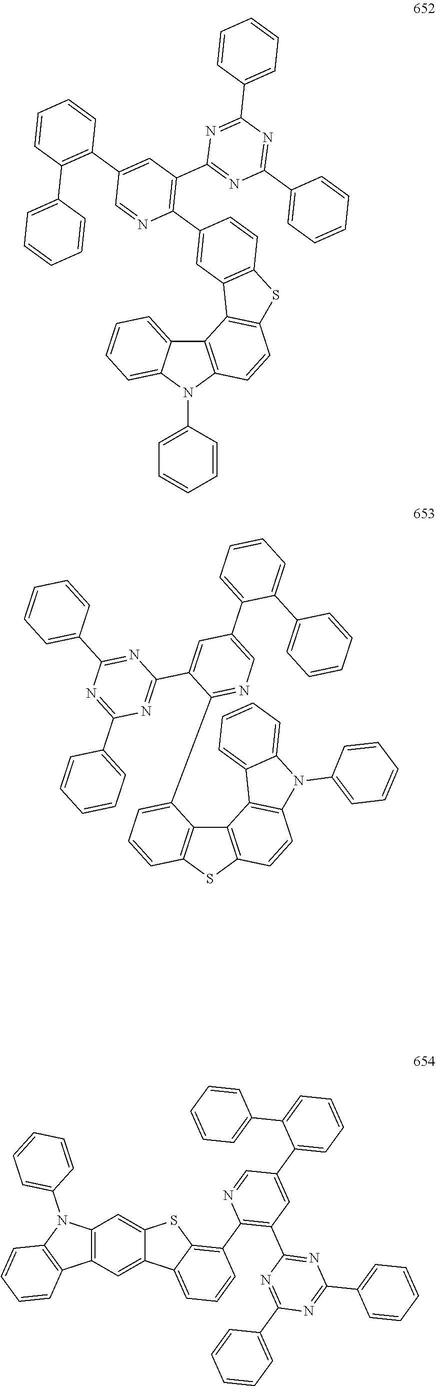 [SMILES: c1ccc(-c2nc(-c3ccccc3)nc(-c3cc(-c4ccccc4-c4ccccc4)cnc3-c3ccc4sc5ccc6c(c7ccccc7n6-c6ccccc6)c5c4c3)n2)cc1 ; c1ccc(-c2nc(-c3ccccc3)nc(-c3cc(-c4ccccc4-c4ccccc4)cnc3-c3cccc4sc5ccc6c(c7ccccc7n6-c6ccccc6)c5c34)n2)cc1 ; c1ccc(-c2nc(-c3ccccc3)nc(-c3cc(-c4ccccc4-c4ccccc4)cnc3-c3cccc4c3sc3cc5c(cc34)c3ccccc3n5-c3ccccc3)n2)cc1]

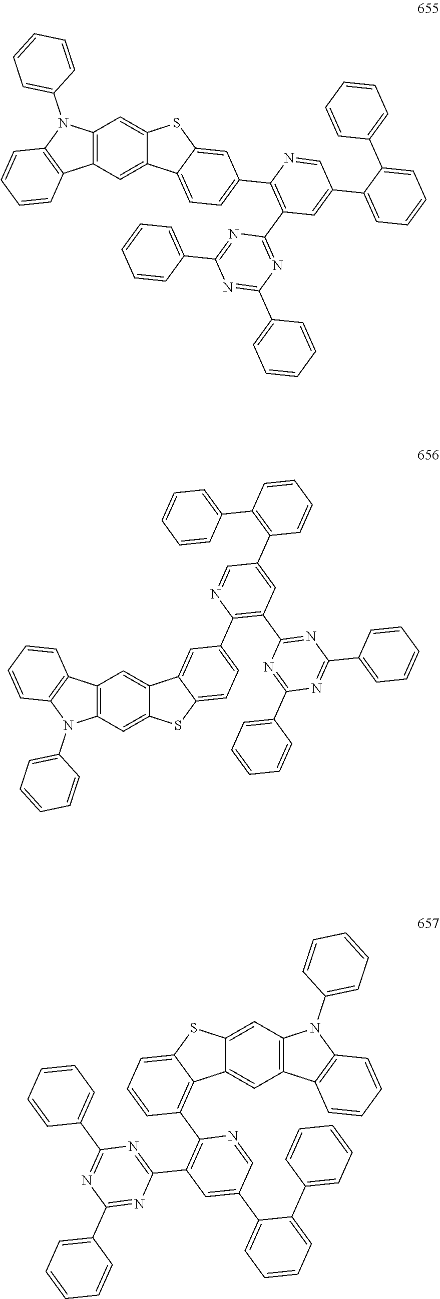 [SMILES: c1ccc(-c2nc(-c3ccccc3)nc(-c3cc(-c4ccccc4-c4ccccc4)cnc3-c3ccc4c(c3)sc3cc5c(cc34)c3ccccc3n5-c3ccccc3)n2)cc1 ; c1ccc(-c2nc(-c3ccccc3)nc(-c3cc(-c4ccccc4-c4ccccc4)cnc3-c3ccc4sc5cc6c(cc5c4c3)c3ccccc3n6-c3ccccc3)n2)cc1 ; c1ccc(-c2nc(-c3ccccc3)nc(-c3cc(-c4ccccc4-c4ccccc4)cnc3-c3cccc4sc5cc6c(cc5c34)c3ccccc3n6-c3ccccc3)n2)cc1]

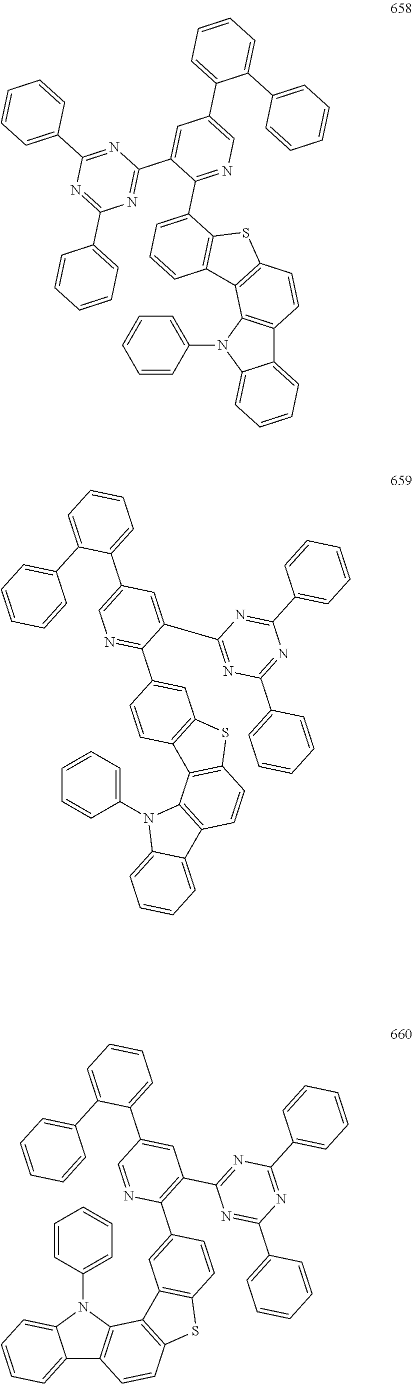 [SMILES: c1ccc(-c2nc(-c3ccccc3)nc(-c3cc(-c4ccccc4-c4ccccc4)cnc3-c3cccc4c3sc3ccc5c6ccccc6n(-c6ccccc6)c5c34)n2)cc1 ; c1ccc(-c2nc(-c3ccccc3)nc(-c3cc(-c4ccccc4-c4ccccc4)cnc3-c3ccc4c(c3)sc3ccc5c6ccccc6n(-c6ccccc6)c5c34)n2)cc1 ; c1ccc(-c2nc(-c3ccccc3)nc(-c3cc(-c4ccccc4-c4ccccc4)cnc3-c3ccc4sc5ccc6c7ccccc7n(-c7ccccc7)c6c5c4c3)n2)cc1]

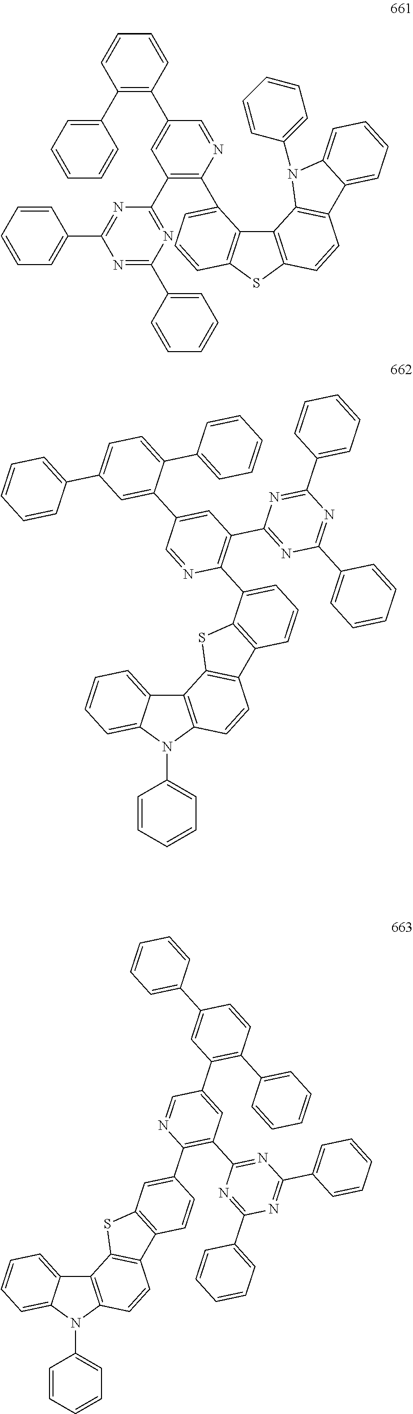 [SMILES: c1ccc(-c2nc(-c3ccccc3)nc(-c3cc(-c4ccccc4-c4ccccc4)cnc3-c3cccc4sc5ccc6c7ccccc7n(-c7ccccc7)c6c5c34)n2)cc1 ; c1ccc(-c2ccc(-c3ccccc3)c(-c3cnc(-c4cccc5c4sc4c5ccc5c4c4ccccc4n5-c4ccccc4)c(-c4nc(-c5ccccc5)nc(-c5ccccc5)n4)c3)c2)cc1 ; c1ccc(-c2ccc(-c3ccccc3)c(-c3cnc(-c4ccc5c(c4)sc4c5ccc5c4c4ccccc4n5-c4ccccc4)c(-c4nc(-c5ccccc5)nc(-c5ccccc5)n4)c3)c2)cc1]

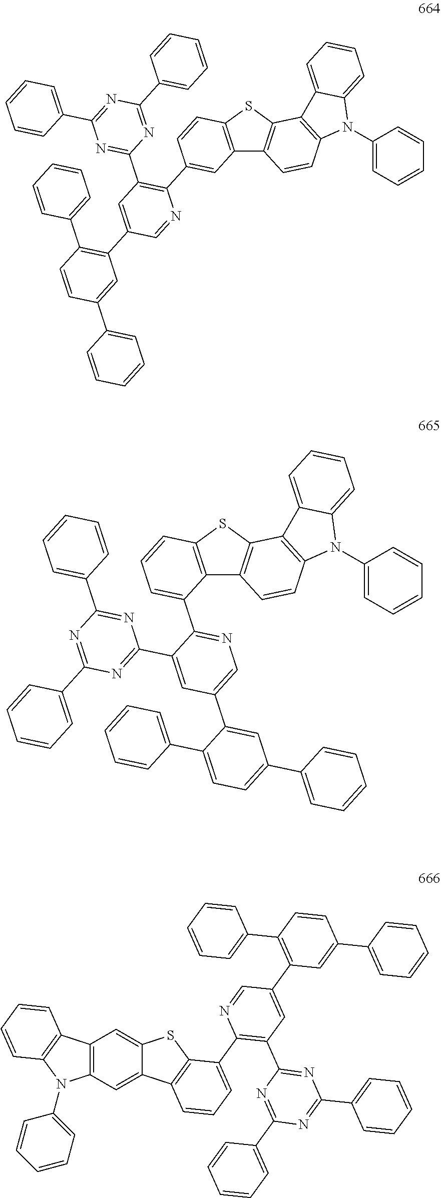 [SMILES: c1ccc(-c2ccc(-c3ccccc3)c(-c3cnc(-c4ccc5sc6c(ccc7c6c6ccccc6n7-c6ccccc6)c5c4)c(-c4nc(-c5ccccc5)nc(-c5ccccc5)n4)c3)c2)cc1 ; c1ccc(-c2ccc(-c3ccccc3)c(-c3cnc(-c4cccc5sc6c(ccc7c6c6ccccc6n7-c6ccccc6)c45)c(-c4nc(-c5ccccc5)nc(-c5ccccc5)n4)c3)c2)cc1 ; c1ccc(-c2ccc(-c3ccccc3)c(-c3cnc(-c4cccc5c4sc4cc6c7ccccc7n(-c7ccccc7)c6cc45)c(-c4nc(-c5ccccc5)nc(-c5ccccc5)n4)c3)c2)cc1]

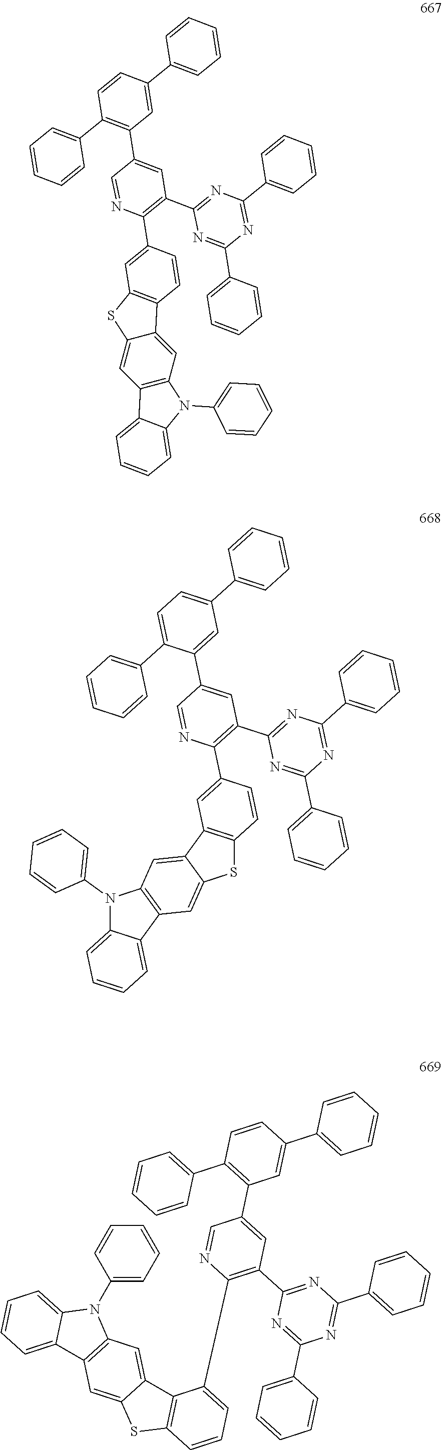 [SMILES: c1ccc(-c2ccc(-c3ccccc3)c(-c3cnc(-c4ccc5c(c4)sc4cc6c7ccccc7n(-c7ccccc7)c6cc45)c(-c4nc(-c5ccccc5)nc(-c5ccccc5)n4)c3)c2)cc1 ; c1ccc(-c2ccc(-c3ccccc3)c(-c3cnc(-c4ccc5sc6cc7c8ccccc8n(-c8ccccc8)c7cc6c5c4)c(-c4nc(-c5ccccc5)nc(-c5ccccc5)n4)c3)c2)cc1 ; c1ccc(-c2ccc(-c3ccccc3)c(-c3cnc(-c4cccc5sc6cc7c8ccccc8n(-c8ccccc8)c7cc6c45)c(-c4nc(-c5ccccc5)nc(-c5ccccc5)n4)c3)c2)cc1]

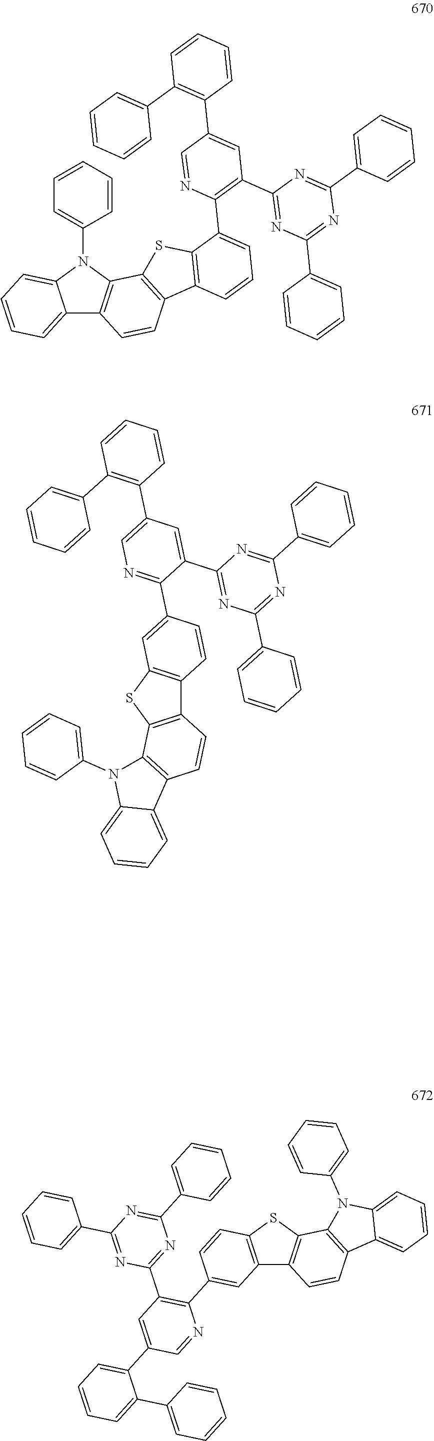 [SMILES: c1ccc(-c2nc(-c3ccccc3)nc(-c3cc(-c4ccccc4-c4ccccc4)cnc3-c3cccc4c3sc3c4ccc4c5ccccc5n(-c5ccccc5)c43)n2)cc1 ; c1ccc(-c2nc(-c3ccccc3)nc(-c3cc(-c4ccccc4-c4ccccc4)cnc3-c3ccc4c(c3)sc3c4ccc4c5ccccc5n(-c5ccccc5)c43)n2)cc1 ; c1ccc(-c2nc(-c3ccccc3)nc(-c3cc(-c4ccccc4-c4ccccc4)cnc3-c3ccc4sc5c(ccc6c7ccccc7n(-c7ccccc7)c65)c4c3)n2)cc1]

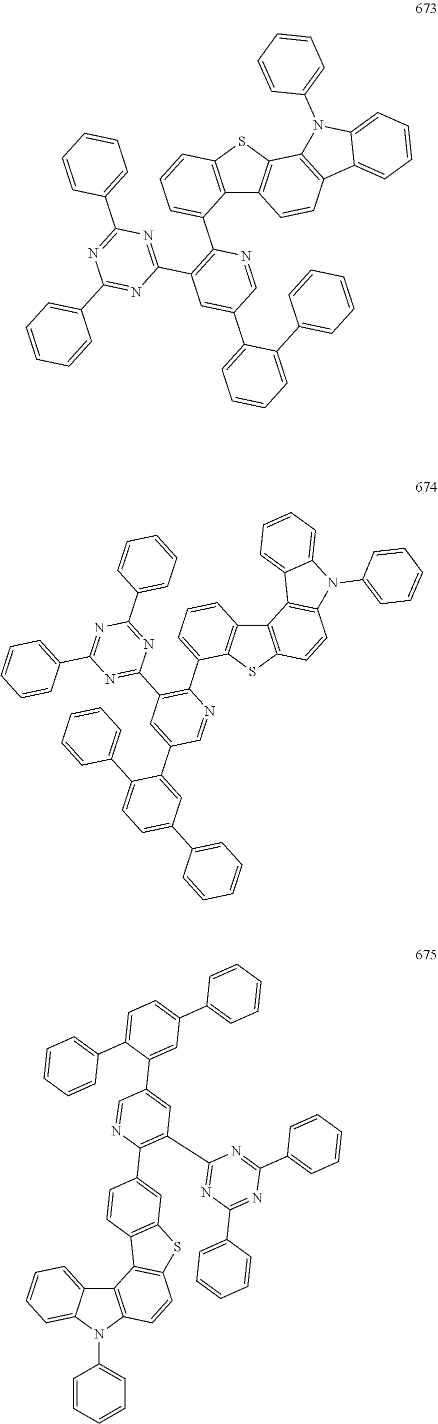 [SMILES: c1ccc(-c2nc(-c3ccccc3)nc(-c3cc(-c4ccccc4-c4ccccc4)cnc3-c3cccc4sc5c(ccc6c7ccccc7n(-c7ccccc7)c65)c34)n2)cc1 ; c1ccc(-c2ccc(-c3ccccc3)c(-c3cnc(-c4cccc5c4sc4ccc6c(c7ccccc7n6-c6ccccc6)c45)c(-c4nc(-c5ccccc5)nc(-c5ccccc5)n4)c3)c2)cc1 ; c1ccc(-c2ccc(-c3ccccc3)c(-c3cnc(-c4ccc5c(c4)sc4ccc6c(c7ccccc7n6-c6ccccc6)c45)c(-c4nc(-c5ccccc5)nc(-c5ccccc5)n4)c3)c2)cc1]

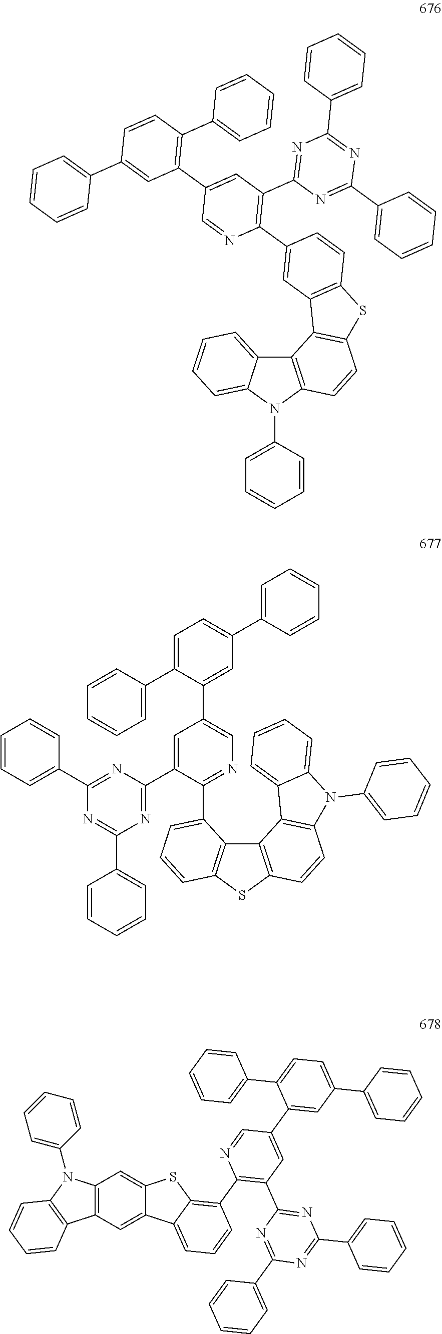 [SMILES: c1ccc(-c2ccc(-c3ccccc3)c(-c3cnc(-c4ccc5sc6ccc7c(c8ccccc8n7-c7ccccc7)c6c5c4)c(-c4nc(-c5ccccc5)nc(-c5ccccc5)n4)c3)c2)cc1 ; c1ccc(-c2ccc(-c3ccccc3)c(-c3cnc(-c4cccc5sc6ccc7c(c8ccccc8n7-c7ccccc7)c6c45)c(-c4nc(-c5ccccc5)nc(-c5ccccc5)n4)c3)c2)cc1 ; c1ccc(-c2ccc(-c3ccccc3)c(-c3cnc(-c4cccc5c4sc4cc6c(cc45)c4ccccc4n6-c4ccccc4)c(-c4nc(-c5ccccc5)nc(-c5ccccc5)n4)c3)c2)cc1]

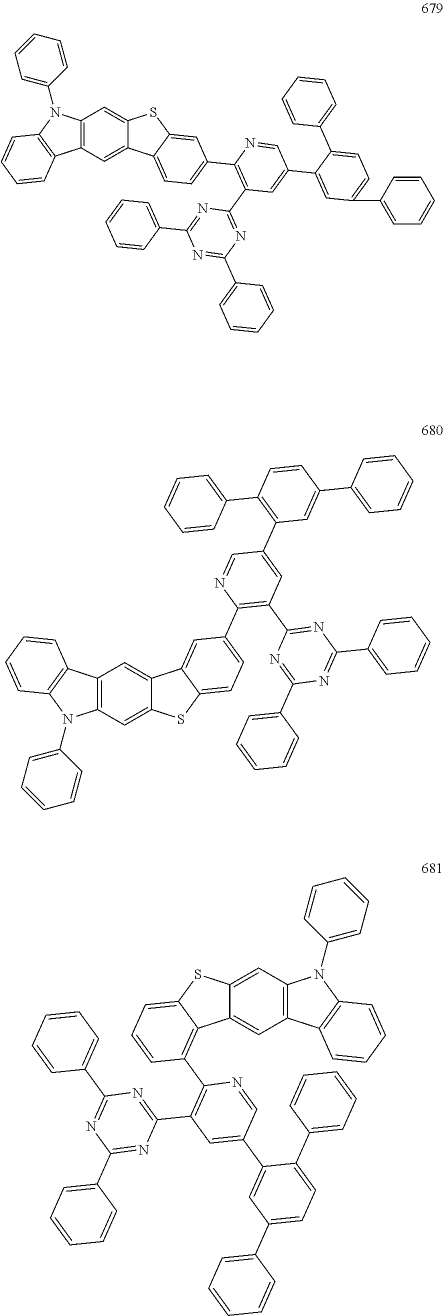 [SMILES: c1ccc(-c2ccc(-c3ccccc3)c(-c3cnc(-c4ccc5c(c4)sc4cc6c(cc45)c4ccccc4n6-c4ccccc4)c(-c4nc(-c5ccccc5)nc(-c5ccccc5)n4)c3)c2)cc1 ; c1ccc(-c2ccc(-c3ccccc3)c(-c3cnc(-c4ccc5sc6cc7c(cc6c5c4)c4ccccc4n7-c4ccccc4)c(-c4nc(-c5ccccc5)nc(-c5ccccc5)n4)c3)c2)cc1 ; c1ccc(-c2ccc(-c3ccccc3)c(-c3cnc(-c4cccc5sc6cc7c(cc6c45)c4ccccc4n7-c4ccccc4)c(-c4nc(-c5ccccc5)nc(-c5ccccc5)n4)c3)c2)cc1]

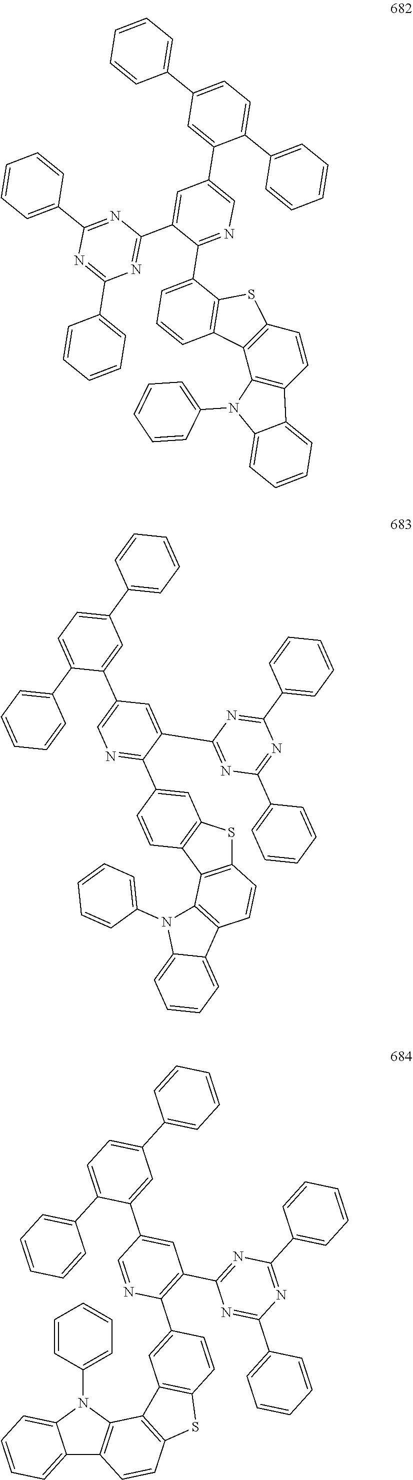 [SMILES: c1ccc(-c2ccc(-c3ccccc3)c(-c3cnc(-c4cccc5c4sc4ccc6c7ccccc7n(-c7ccccc7)c6c45)c(-c4nc(-c5ccccc5)nc(-c5ccccc5)n4)c3)c2)cc1 ; c1ccc(-c2ccc(-c3ccccc3)c(-c3cnc(-c4ccc5c(c4)sc4ccc6c7ccccc7n(-c7ccccc7)c6c45)c(-c4nc(-c5ccccc5)nc(-c5ccccc5)n4)c3)c2)cc1 ; c1ccc(-c2ccc(-c3ccccc3)c(-c3cnc(-c4ccc5sc6ccc7c8ccccc8n(-c8ccccc8)c7c6c5c4)c(-c4nc(-c5ccccc5)nc(-c5ccccc5)n4)c3)c2)cc1]

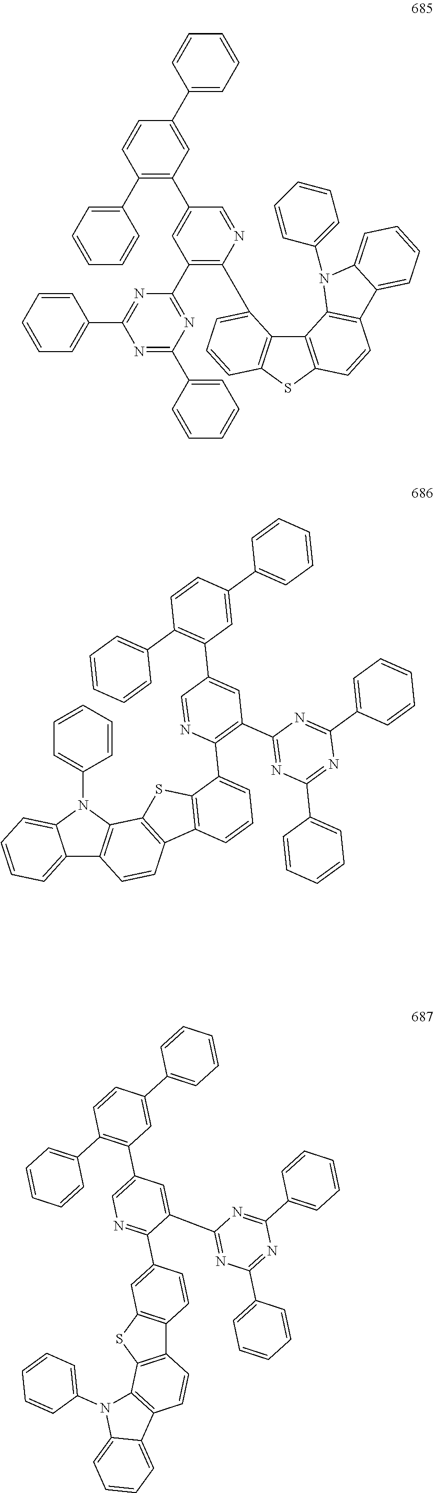 [SMILES: c1ccc(-c2ccc(-c3ccccc3)c(-c3cnc(-c4cccc5sc6ccc7c8ccccc8n(-c8ccccc8)c7c6c45)c(-c4nc(-c5ccccc5)nc(-c5ccccc5)n4)c3)c2)cc1 ; c1ccc(-c2ccc(-c3ccccc3)c(-c3cnc(-c4cccc5c4sc4c5ccc5c6ccccc6n(-c6ccccc6)c54)c(-c4nc(-c5ccccc5)nc(-c5ccccc5)n4)c3)c2)cc1 ; c1ccc(-c2ccc(-c3ccccc3)c(-c3cnc(-c4ccc5c(c4)sc4c5ccc5c6ccccc6n(-c6ccccc6)c54)c(-c4nc(-c5ccccc5)nc(-c5ccccc5)n4)c3)c2)cc1]

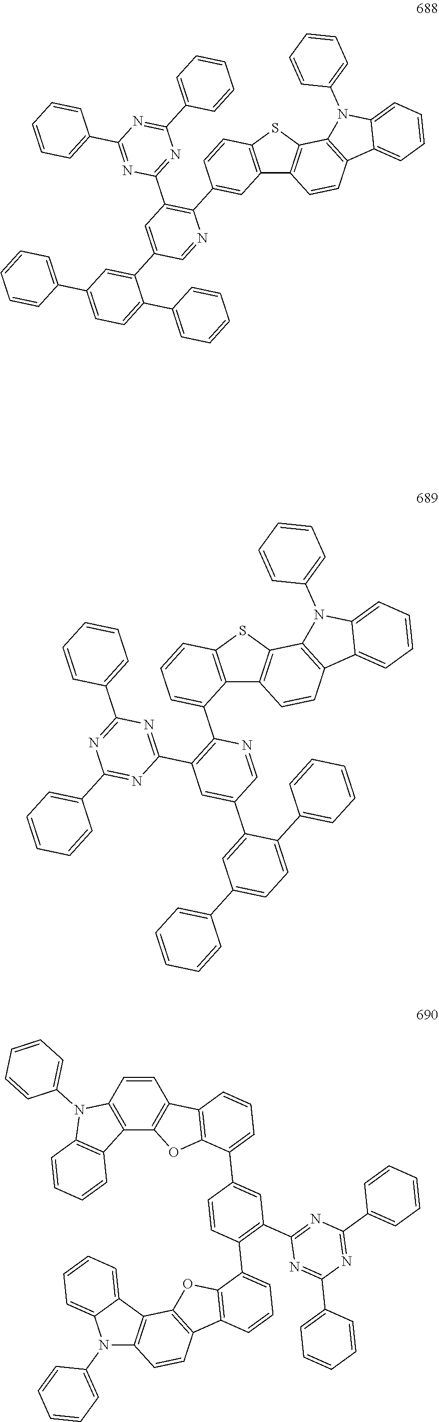 [SMILES: c1ccc(-c2ccc(-c3ccccc3)c(-c3cnc(-c4ccc5sc6c(ccc7c8ccccc8n(-c8ccccc8)c76)c5c4)c(-c4nc(-c5ccccc5)nc(-c5ccccc5)n4)c3)c2)cc1 ; c1ccc(-c2ccc(-c3ccccc3)c(-c3cnc(-c4cccc5sc6c(ccc7c8ccccc8n(-c8ccccc8)c76)c45)c(-c4nc(-c5ccccc5)nc(-c5ccccc5)n4)c3)c2)cc1 ; c1ccc(-c2nc(-c3ccccc3)nc(-c3cc(-c4cccc5c4oc4c5ccc5c4c4ccccc4n5-c4ccccc4)ccc3-c3cccc4c3oc3c4ccc4c3c3ccccc3n4-c3ccccc3)n2)cc1]

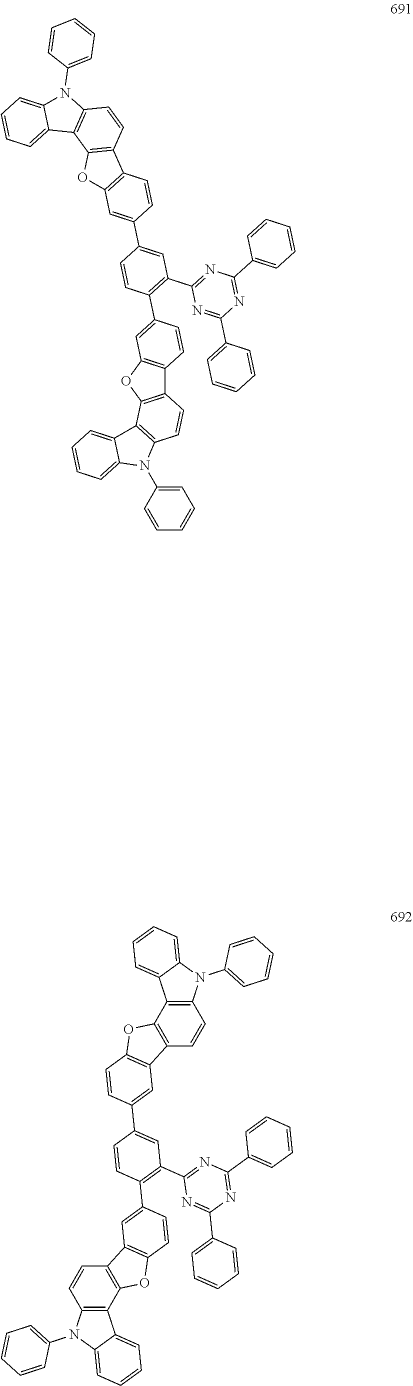 [SMILES: c1ccc(-c2nc(-c3ccccc3)nc(-c3cc(-c4ccc5c(c4)oc4c5ccc5c4c4ccccc4n5-c4ccccc4)ccc3-c3ccc4c(c3)oc3c4ccc4c3c3ccccc3n4-c3ccccc3)n2)cc1 ; c1ccc(-c2nc(-c3ccccc3)nc(-c3cc(-c4ccc5oc6c(ccc7c6c6ccccc6n7-c6ccccc6)c5c4)ccc3-c3ccc4oc5c(ccc6c5c5ccccc5n6-c5ccccc5)c4c3)n2)cc1]

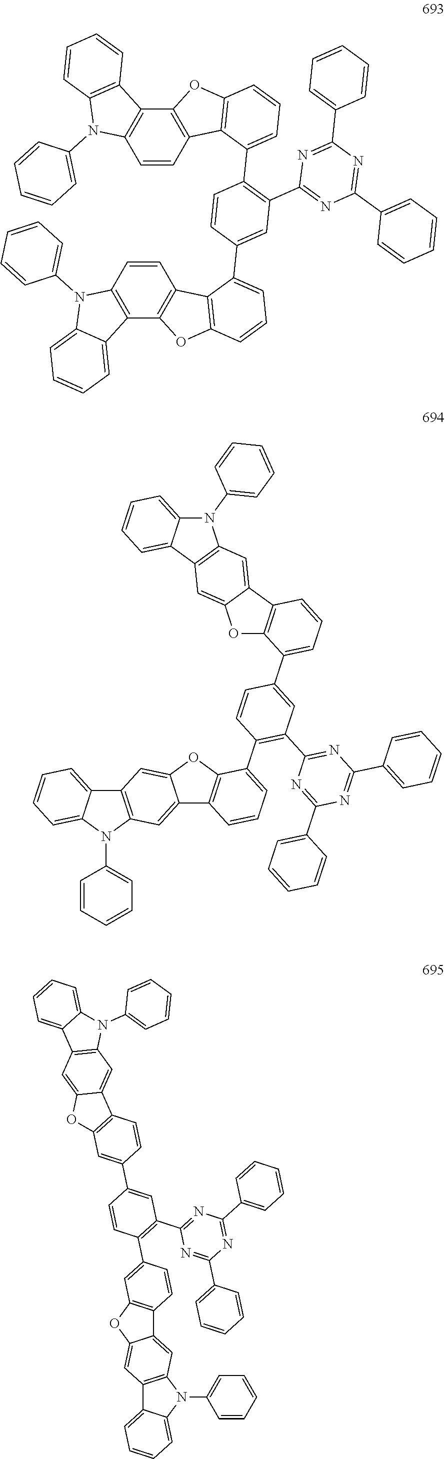 [SMILES: c1ccc(-c2nc(-c3ccccc3)nc(-c3cc(-c4cccc5oc6c(ccc7c6c6ccccc6n7-c6ccccc6)c45)ccc3-c3cccc4oc5c(ccc6c5c5ccccc5n6-c5ccccc5)c34)n2)cc1 ; c1ccc(-c2nc(-c3ccccc3)nc(-c3cc(-c4cccc5c4oc4cc6c7ccccc7n(-c7ccccc7)c6cc45)ccc3-c3cccc4c3oc3cc5c6ccccc6n(-c6ccccc6)c5cc34)n2)cc1 ; c1ccc(-c2nc(-c3ccccc3)nc(-c3cc(-c4ccc5c(c4)oc4cc6c7ccccc7n(-c7ccccc7)c6cc45)ccc3-c3ccc4c(c3)oc3cc5c6ccccc6n(-c6ccccc6)c5cc34)n2)cc1]

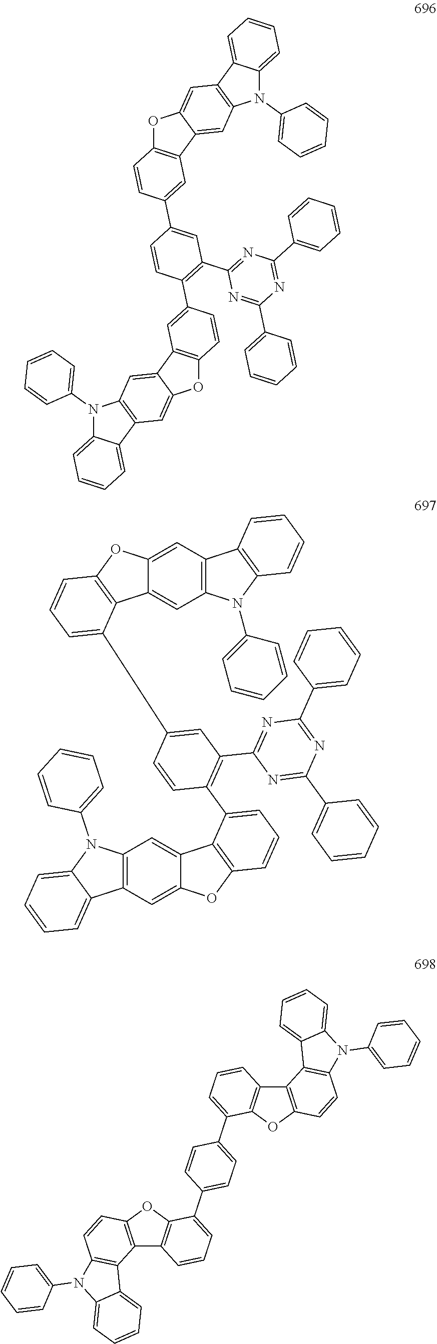 [SMILES: c1ccc(-c2nc(-c3ccccc3)nc(-c3cc(-c4ccc5oc6cc7c8ccccc8n(-c8ccccc8)c7cc6c5c4)ccc3-c3ccc4oc5cc6c7ccccc7n(-c7ccccc7)c6cc5c4c3)n2)cc1 ; c1ccc(-c2nc(-c3ccccc3)nc(-c3cc(-c4cccc5oc6cc7c8ccccc8n(-c8ccccc8)c7cc6c45)ccc3-c3cccc4oc5cc6c7ccccc7n(-c7ccccc7)c6cc5c34)n2)cc1 ; c1ccc(-n2c3ccccc3c3c4c(ccc32)oc2c(-c3ccc(-c5cccc6c5oc5ccc7c(c8ccccc8n7-c7ccccc7)c56)cc3)cccc24)cc1]

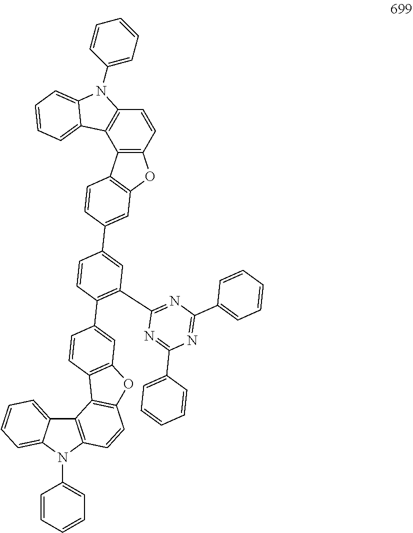 [SMILES: c1ccc(-c2nc(-c3ccccc3)nc(-c3cc(-c4ccc5c(c4)oc4ccc6c(c7ccccc7n6-c6ccccc6)c45)ccc3-c3ccc4c(c3)oc3ccc5c(c6ccccc6n5-c5ccccc5)c34)n2)cc1]

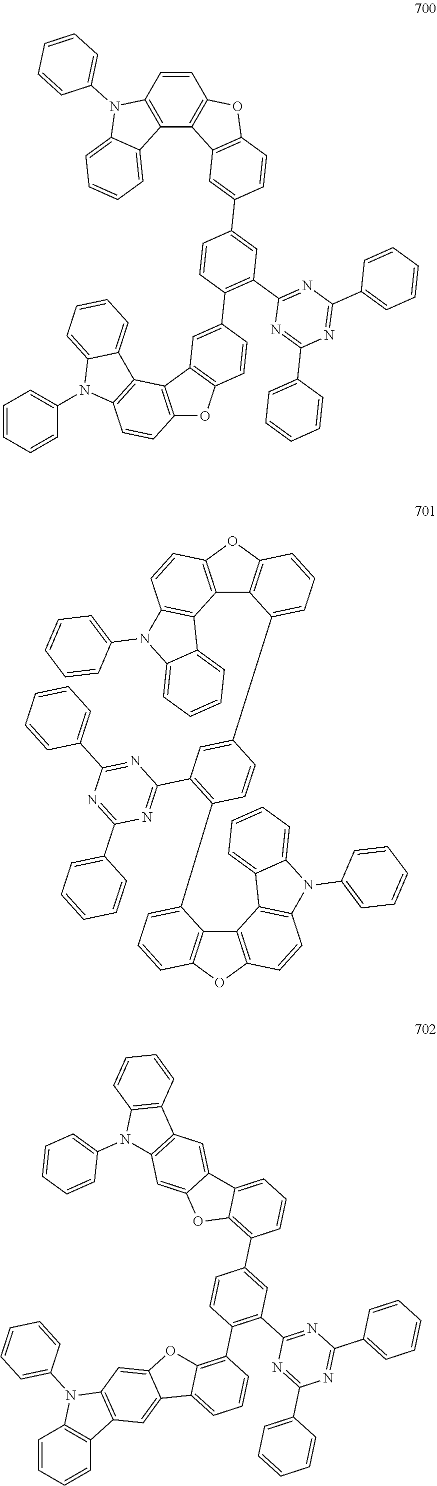 [SMILES: c1ccc(-c2nc(-c3ccccc3)nc(-c3cc(-c4ccc5oc6ccc7c(c8ccccc8n7-c7ccccc7)c6c5c4)ccc3-c3ccc4oc5ccc6c(c7ccccc7n6-c6ccccc6)c5c4c3)n2)cc1 ; c1ccc(-c2nc(-c3ccccc3)nc(-c3cc(-c4cccc5oc6ccc7c(c8ccccc8n7-c7ccccc7)c6c45)ccc3-c3cccc4oc5ccc6c(c7ccccc7n6-c6ccccc6)c5c34)n2)cc1 ; c1ccc(-c2nc(-c3ccccc3)nc(-c3cc(-c4cccc5c4oc4cc6c(cc45)c4ccccc4n6-c4ccccc4)ccc3-c3cccc4c3oc3cc5c(cc34)c3ccccc3n5-c3ccccc3)n2)cc1]

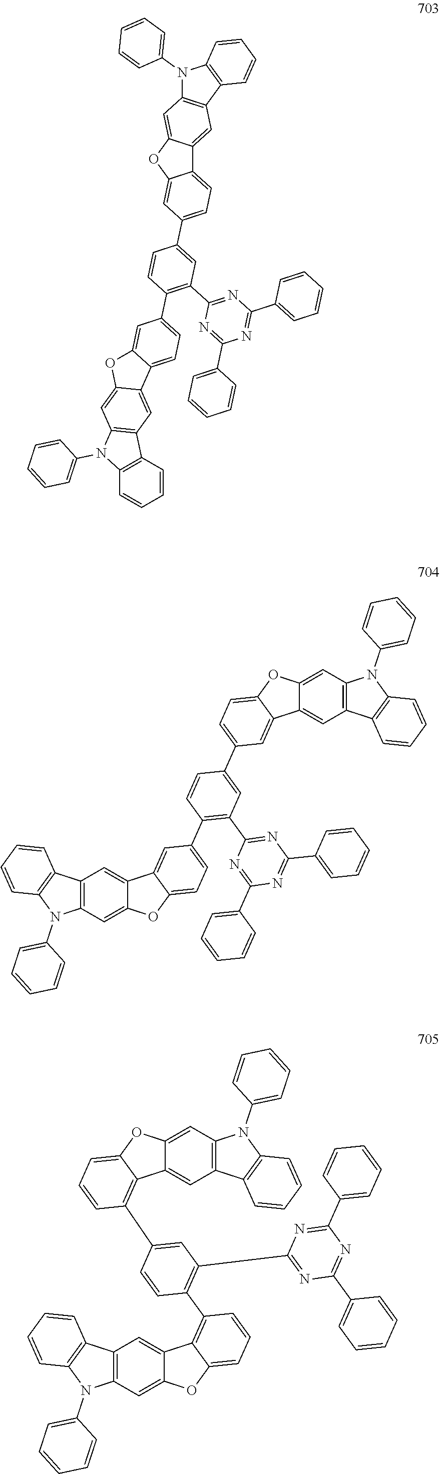 [SMILES: c1ccc(-c2nc(-c3ccccc3)nc(-c3cc(-c4ccc5c(c4)oc4cc6c(cc45)c4ccccc4n6-c4ccccc4)ccc3-c3ccc4c(c3)oc3cc5c(cc34)c3ccccc3n5-c3ccccc3)n2)cc1 ; c1ccc(-c2nc(-c3ccccc3)nc(-c3cc(-c4ccc5oc6cc7c(cc6c5c4)c4ccccc4n7-c4ccccc4)ccc3-c3ccc4oc5cc6c(cc5c4c3)c3ccccc3n6-c3ccccc3)n2)cc1 ; c1ccc(-c2nc(-c3ccccc3)nc(-c3cc(-c4cccc5oc6cc7c(cc6c45)c4ccccc4n7-c4ccccc4)ccc3-c3cccc4oc5cc6c(cc5c34)c3ccccc3n6-c3ccccc3)n2)cc1]

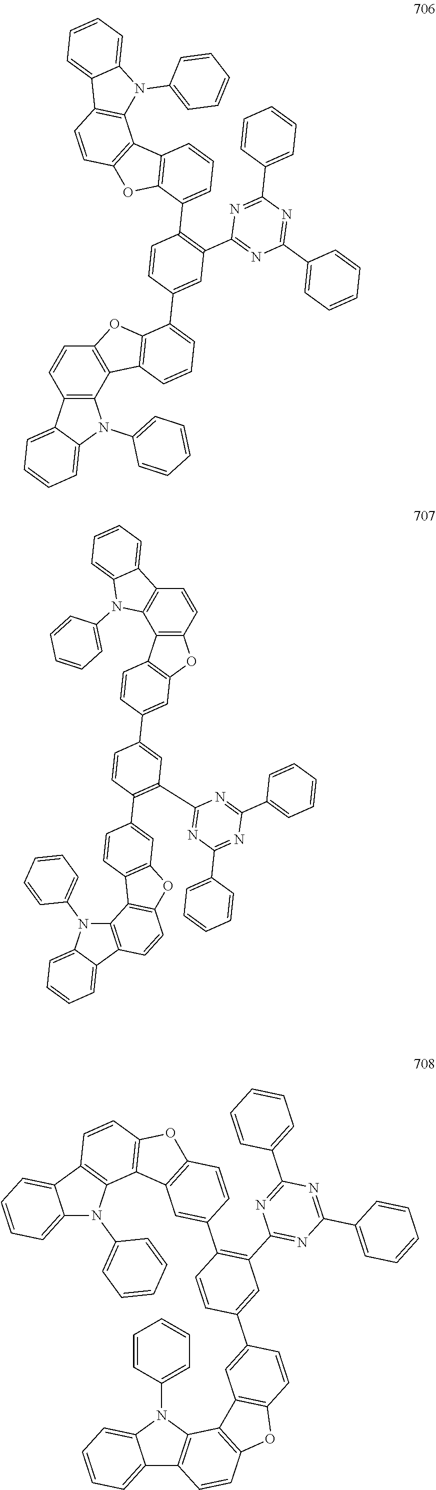 [SMILES: c1ccc(-c2nc(-c3ccccc3)nc(-c3cc(-c4cccc5c4oc4ccc6c7ccccc7n(-c7ccccc7)c6c45)ccc3-c3cccc4c3oc3ccc5c6ccccc6n(-c6ccccc6)c5c34)n2)cc1 ; c1ccc(-c2nc(-c3ccccc3)nc(-c3cc(-c4ccc5c(c4)oc4ccc6c7ccccc7n(-c7ccccc7)c6c45)ccc3-c3ccc4c(c3)oc3ccc5c6ccccc6n(-c6ccccc6)c5c34)n2)cc1 ; c1ccc(-c2nc(-c3ccccc3)nc(-c3cc(-c4ccc5oc6ccc7c8ccccc8n(-c8ccccc8)c7c6c5c4)ccc3-c3ccc4oc5ccc6c7ccccc7n(-c7ccccc7)c6c5c4c3)n2)cc1]

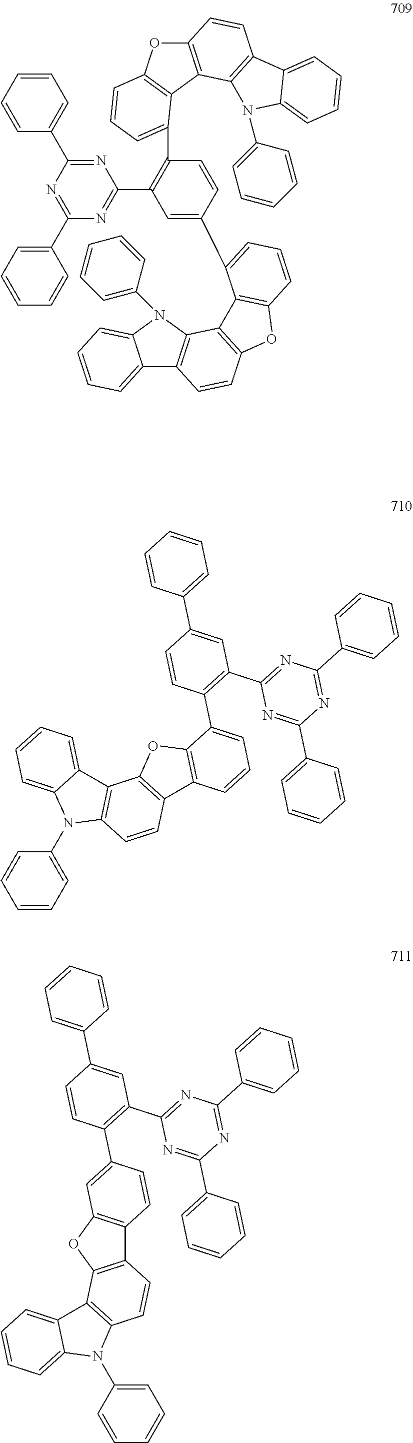 [SMILES: c1ccc(-c2nc(-c3ccccc3)nc(-c3cc(-c4cccc5oc6ccc7c8ccccc8n(-c8ccccc8)c7c6c45)ccc3-c3cccc4oc5ccc6c7ccccc7n(-c7ccccc7)c6c5c34)n2)cc1 ; c1ccc(-c2ccc(-c3cccc4c3oc3c4ccc4c3c3ccccc3n4-c3ccccc3)c(-c3nc(-c4ccccc4)nc(-c4ccccc4)n3)c2)cc1 ; c1ccc(-c2ccc(-c3ccc4c(c3)oc3c4ccc4c3c3ccccc3n4-c3ccccc3)c(-c3nc(-c4ccccc4)nc(-c4ccccc4)n3)c2)cc1]

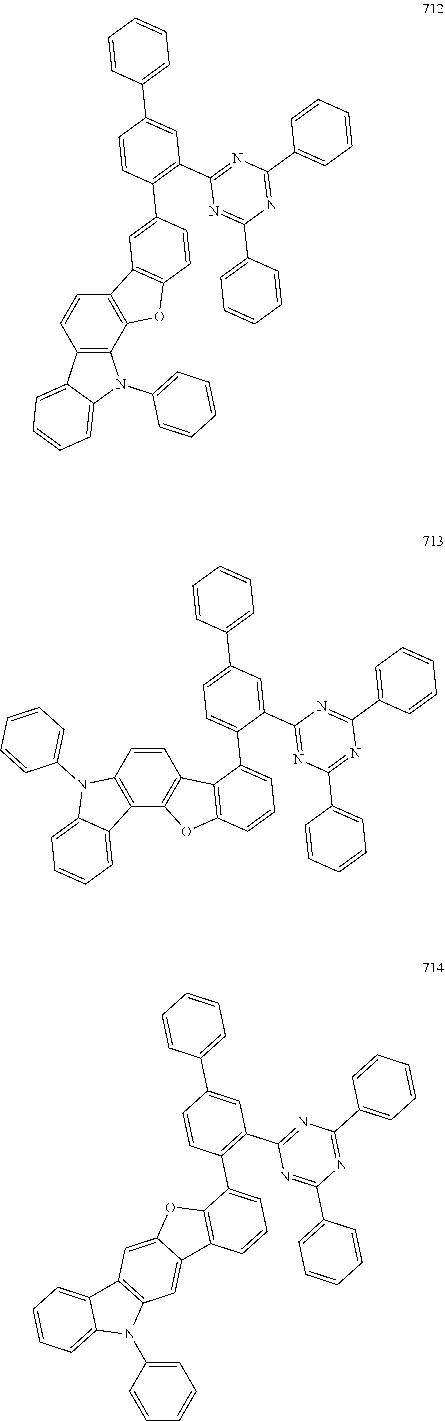 [SMILES: c1ccc(-c2ccc(-c3ccc4oc5c(ccc6c7ccccc7n(-c7ccccc7)c65)c4c3)c(-c3nc(-c4ccccc4)nc(-c4ccccc4)n3)c2)cc1 ; c1ccc(-c2ccc(-c3cccc4oc5c(ccc6c5c5ccccc5n6-c5ccccc5)c34)c(-c3nc(-c4ccccc4)nc(-c4ccccc4)n3)c2)cc1 ; c1ccc(-c2ccc(-c3cccc4c3oc3cc5c6ccccc6n(-c6ccccc6)c5cc34)c(-c3nc(-c4ccccc4)nc(-c4ccccc4)n3)c2)cc1]

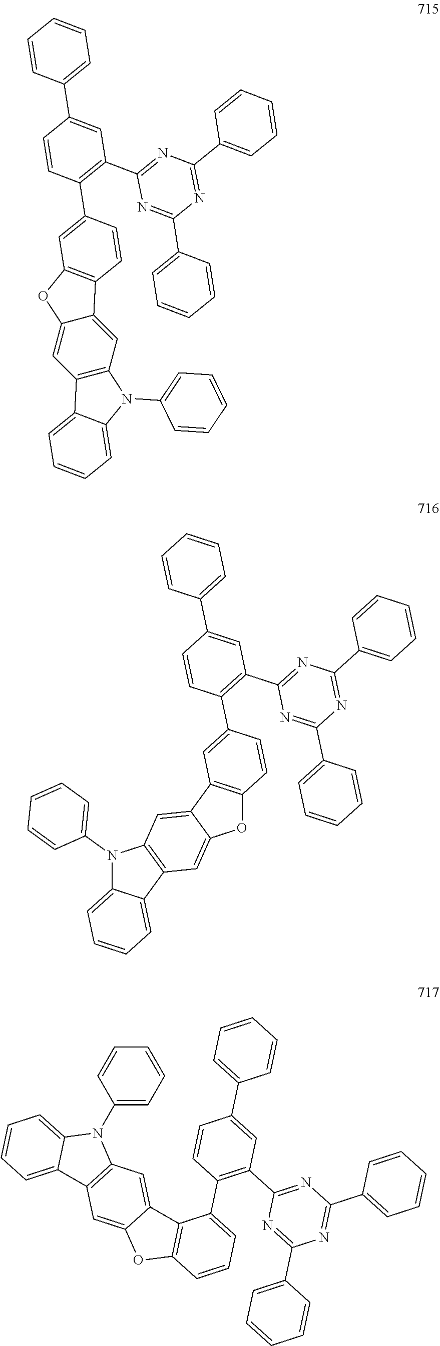 [SMILES: c1ccc(-c2ccc(-c3ccc4c(c3)oc3cc5c6ccccc6n(-c6ccccc6)c5cc34)c(-c3nc(-c4ccccc4)nc(-c4ccccc4)n3)c2)cc1 ; c1ccc(-c2ccc(-c3ccc4oc5cc6c7ccccc7n(-c7ccccc7)c6cc5c4c3)c(-c3nc(-c4ccccc4)nc(-c4ccccc4)n3)c2)cc1 ; c1ccc(-c2ccc(-c3cccc4oc5cc6c7ccccc7n(-c7ccccc7)c6cc5c34)c(-c3nc(-c4ccccc4)nc(-c4ccccc4)n3)c2)cc1]

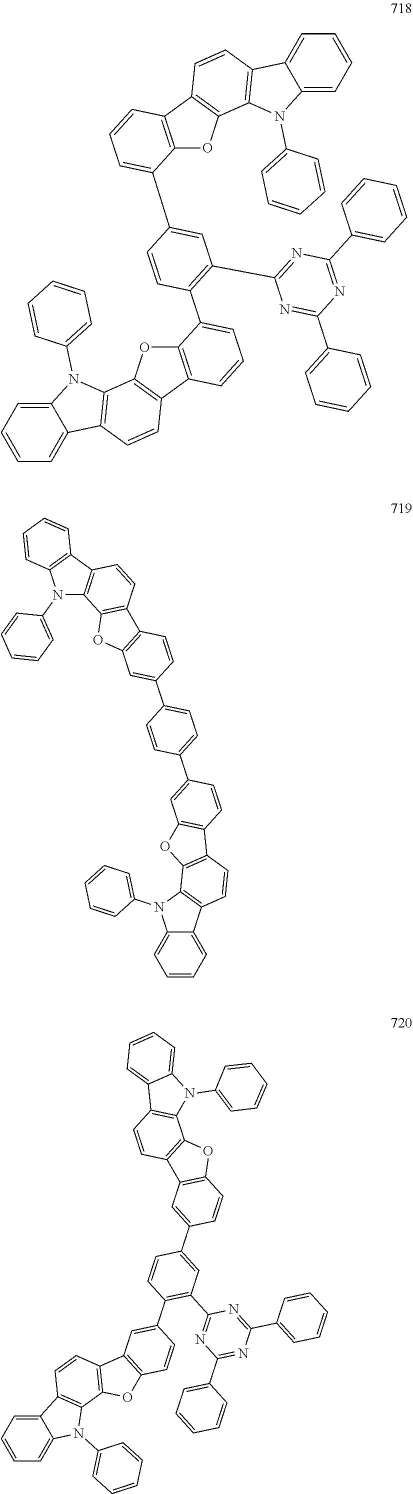 [SMILES: c1ccc(-c2nc(-c3ccccc3)nc(-c3cc(-c4cccc5c4oc4c5ccc5c6ccccc6n(-c6ccccc6)c54)ccc3-c3cccc4c3oc3c4ccc4c5ccccc5n(-c5ccccc5)c43)n2)cc1 ; c1ccc(-n2c3ccccc3c3ccc4c5ccc(-c6ccc(-c7ccc8c(c7)oc7c8ccc8c9ccccc9n(-c9ccccc9)c87)cc6)cc5oc4c32)cc1 ; c1ccc(-c2nc(-c3ccccc3)nc(-c3cc(-c4ccc5oc6c(ccc7c8ccccc8n(-c8ccccc8)c76)c5c4)ccc3-c3ccc4oc5c(ccc6c7ccccc7n(-c7ccccc7)c65)c4c3)n2)cc1]

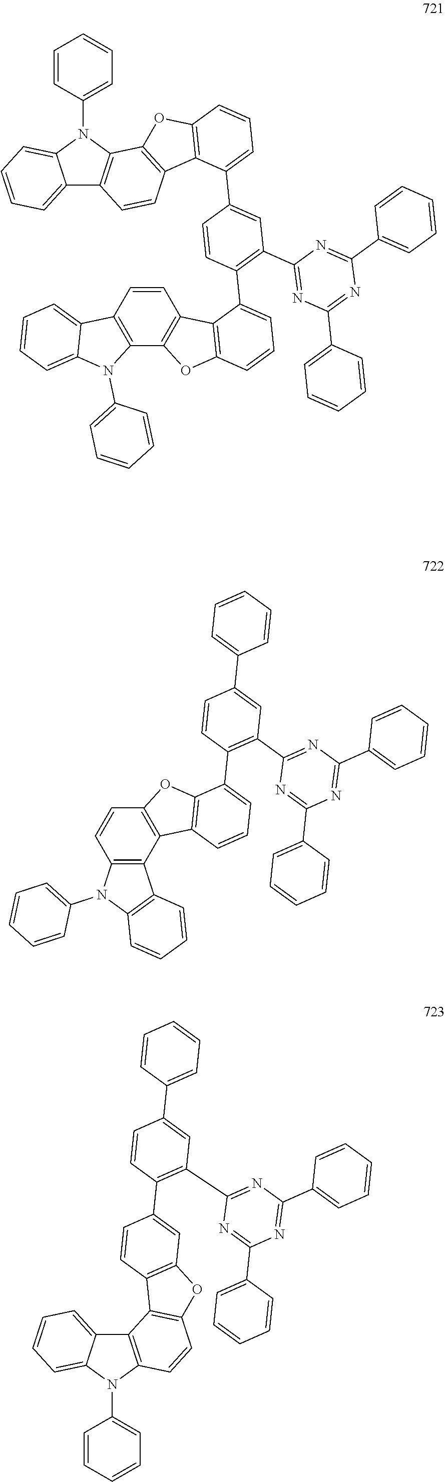 [SMILES: c1ccc(-c2nc(-c3ccccc3)nc(-c3cc(-c4cccc5oc6c(ccc7c8ccccc8n(-c8ccccc8)c76)c45)ccc3-c3cccc4oc5c(ccc6c7ccccc7n(-c7ccccc7)c65)c34)n2)cc1 ; c1ccc(-c2ccc(-c3cccc4c3oc3ccc5c(c6ccccc6n5-c5ccccc5)c34)c(-c3nc(-c4ccccc4)nc(-c4ccccc4)n3)c2)cc1 ; c1ccc(-c2ccc(-c3ccc4c(c3)oc3ccc5c(c6ccccc6n5-c5ccccc5)c34)c(-c3nc(-c4ccccc4)nc(-c4ccccc4)n3)c2)cc1]

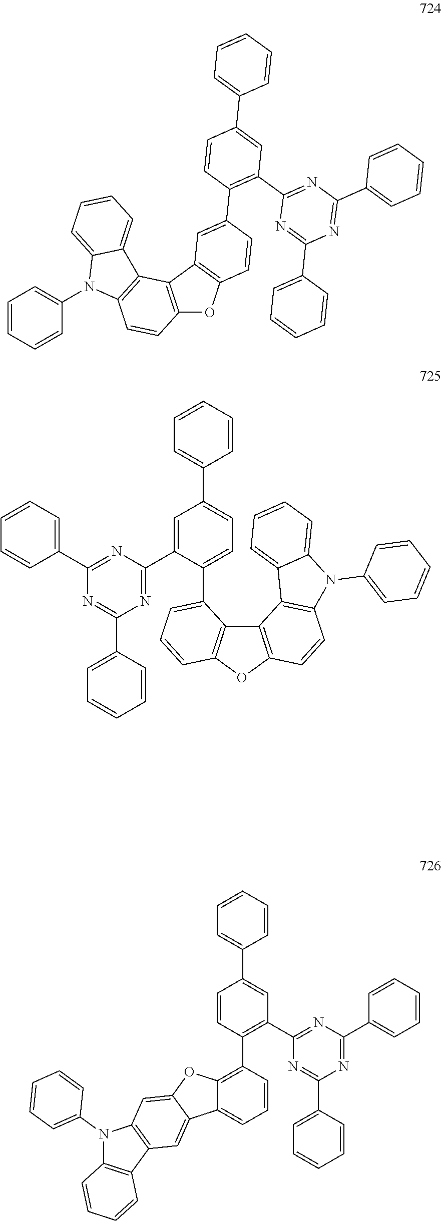 [SMILES: c1ccc(-c2ccc(-c3ccc4oc5ccc6c(c7ccccc7n6-c6ccccc6)c5c4c3)c(-c3nc(-c4ccccc4)nc(-c4ccccc4)n3)c2)cc1 ; c1ccc(-c2ccc(-c3cccc4oc5ccc6c(c7ccccc7n6-c6ccccc6)c5c34)c(-c3nc(-c4ccccc4)nc(-c4ccccc4)n3)c2)cc1 ; c1ccc(-c2ccc(-c3cccc4c3oc3cc5c(cc34)c3ccccc3n5-c3ccccc3)c(-c3nc(-c4ccccc4)nc(-c4ccccc4)n3)c2)cc1]

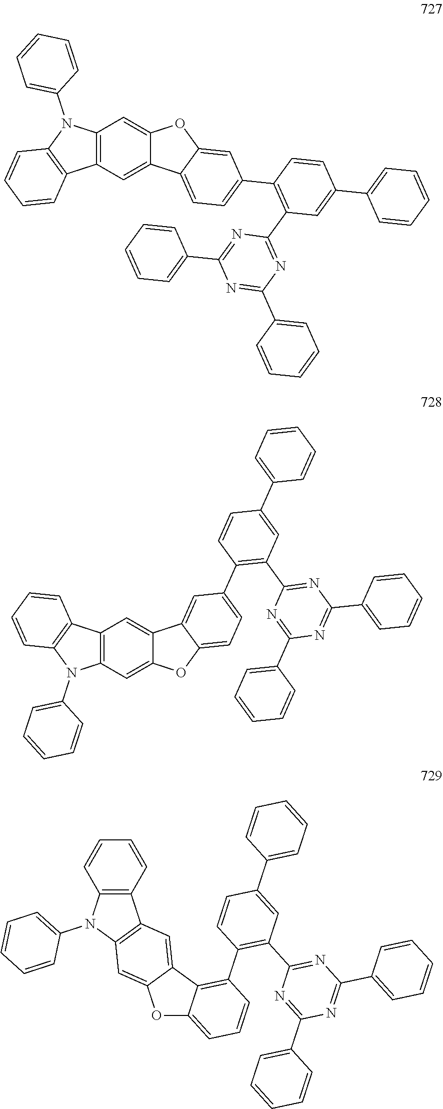 [SMILES: c1ccc(-c2ccc(-c3ccc4c(c3)oc3cc5c(cc34)c3ccccc3n5-c3ccccc3)c(-c3nc(-c4ccccc4)nc(-c4ccccc4)n3)c2)cc1 ; c1ccc(-c2ccc(-c3ccc4oc5cc6c(cc5c4c3)c3ccccc3n6-c3ccccc3)c(-c3nc(-c4ccccc4)nc(-c4ccccc4)n3)c2)cc1 ; c1ccc(-c2ccc(-c3cccc4oc5cc6c(cc5c34)c3ccccc3n6-c3ccccc3)c(-c3nc(-c4ccccc4)nc(-c4ccccc4)n3)c2)cc1]

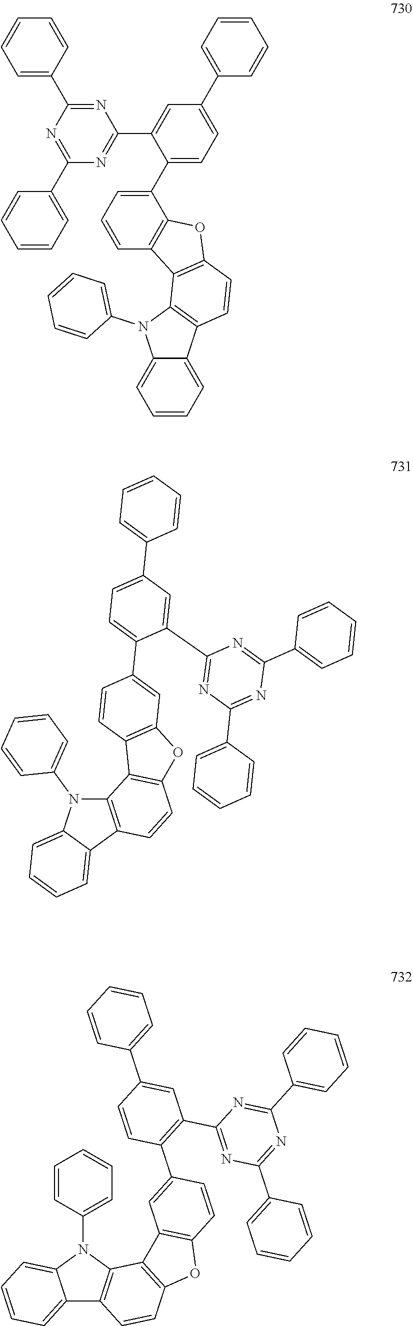 [SMILES: c1ccc(-c2ccc(-c3cccc4c3oc3ccc5c6ccccc6n(-c6ccccc6)c5c34)c(-c3nc(-c4ccccc4)nc(-c4ccccc4)n3)c2)cc1 ; c1ccc(-c2ccc(-c3ccc4c(c3)oc3ccc5c6ccccc6n(-c6ccccc6)c5c34)c(-c3nc(-c4ccccc4)nc(-c4ccccc4)n3)c2)cc1 ; c1ccc(-c2ccc(-c3ccc4oc5ccc6c7ccccc7n(-c7ccccc7)c6c5c4c3)c(-c3nc(-c4ccccc4)nc(-c4ccccc4)n3)c2)cc1]

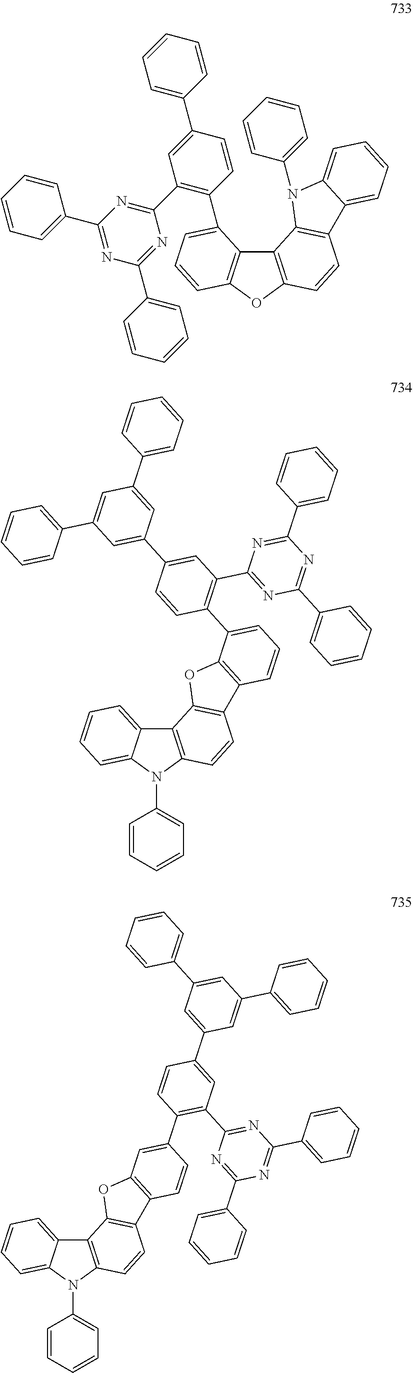

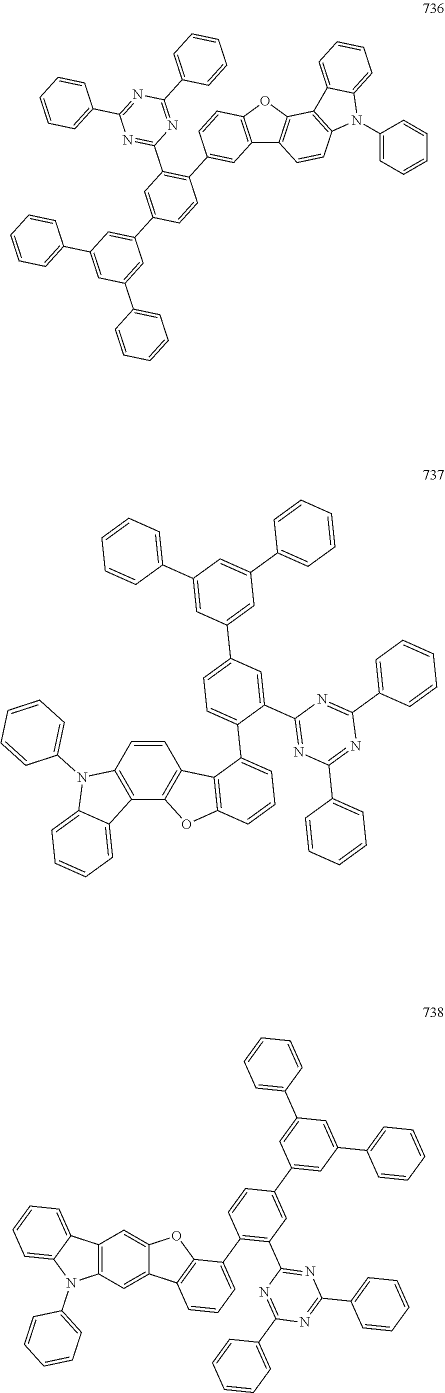 [SMILES: c1ccc(-c2cc(-c3ccccc3)cc(-c3ccc(-c4ccc5oc6c(ccc7c6c6ccccc6n7-c6ccccc6)c5c4)c(-c4nc(-c5ccccc5)nc(-c5ccccc5)n4)c3)c2)cc1 ; c1ccc(-c2cc(-c3ccccc3)cc(-c3ccc(-c4cccc5oc6c(ccc7c6c6ccccc6n7-c6ccccc6)c45)c(-c4nc(-c5ccccc5)nc(-c5ccccc5)n4)c3)c2)cc1 ; c1ccc(-c2cc(-c3ccccc3)cc(-c3ccc(-c4cccc5c4oc4cc6c7ccccc7n(-c7ccccc7)c6cc45)c(-c4nc(-c5ccccc5)nc(-c5ccccc5)n4)c3)c2)cc1]

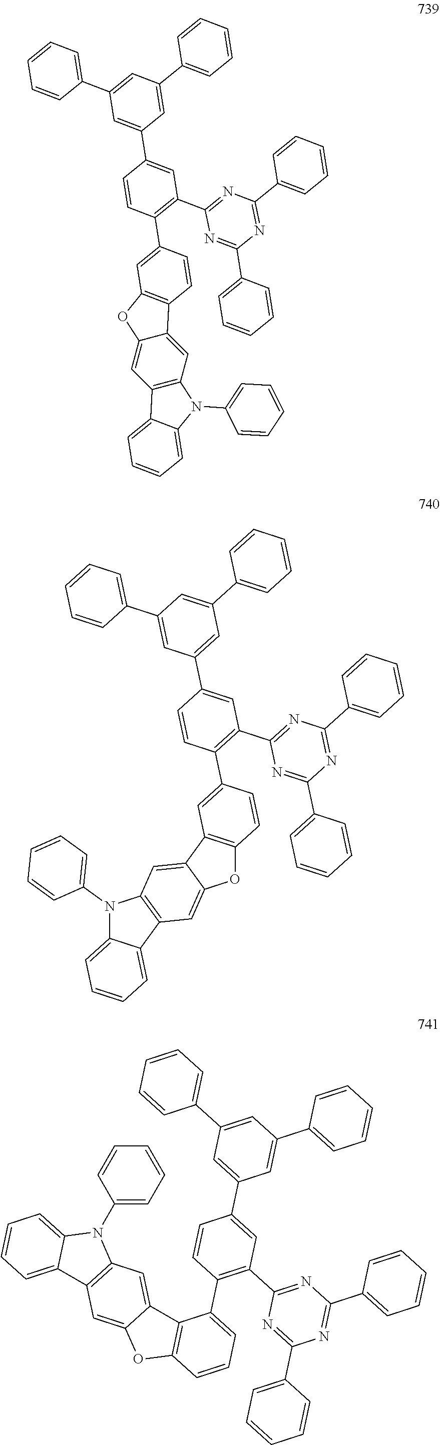 [SMILES: c1ccc(-c2cc(-c3ccccc3)cc(-c3ccc(-c4ccc5c(c4)oc4cc6c7ccccc7n(-c7ccccc7)c6cc45)c(-c4nc(-c5ccccc5)nc(-c5ccccc5)n4)c3)c2)cc1 ; c1ccc(-c2cc(-c3ccccc3)cc(-c3ccc(-c4ccc5oc6cc7c8ccccc8n(-c8ccccc8)c7cc6c5c4)c(-c4nc(-c5ccccc5)nc(-c5ccccc5)n4)c3)c2)cc1 ; c1ccc(-c2cc(-c3ccccc3)cc(-c3ccc(-c4cccc5oc6cc7c8ccccc8n(-c8ccccc8)c7cc6c45)c(-c4nc(-c5ccccc5)nc(-c5ccccc5)n4)c3)c2)cc1]

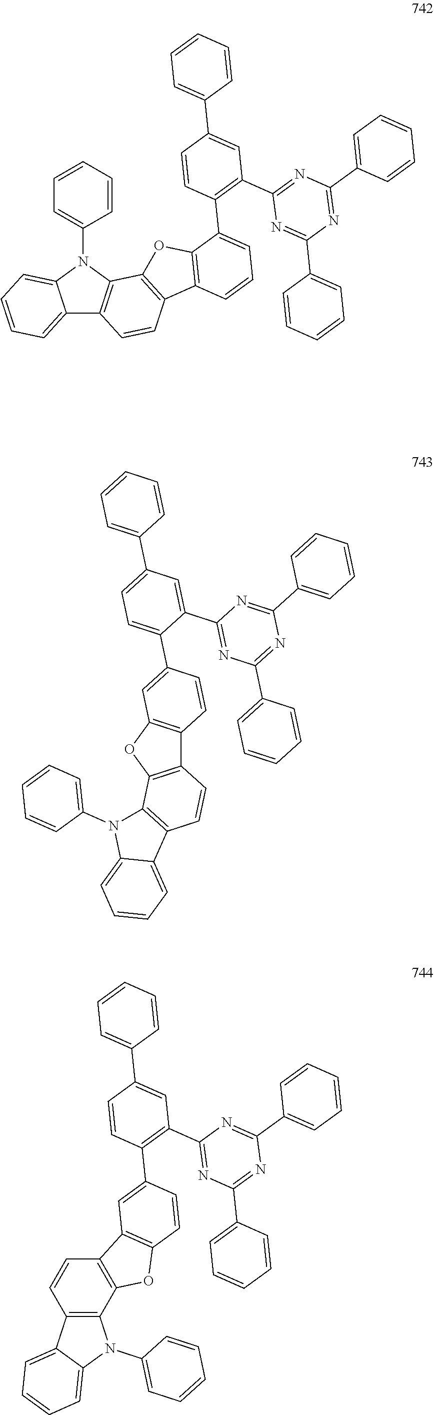 [SMILES: c1ccc(-c2ccc(-c3cccc4c3oc3c4ccc4c5ccccc5n(-c5ccccc5)c43)c(-c3nc(-c4ccccc4)nc(-c4ccccc4)n3)c2)cc1 ; c1ccc(-c2ccc(-c3ccc4c(c3)oc3c4ccc4c5ccccc5n(-c5ccccc5)c43)c(-c3nc(-c4ccccc4)nc(-c4ccccc4)n3)c2)cc1 ; c1ccc(-c2ccc(-c3ccc4oc5c(ccc6c7ccccc7n(-c7ccccc7)c65)c4c3)c(-c3nc(-c4ccccc4)nc(-c4ccccc4)n3)c2)cc1]

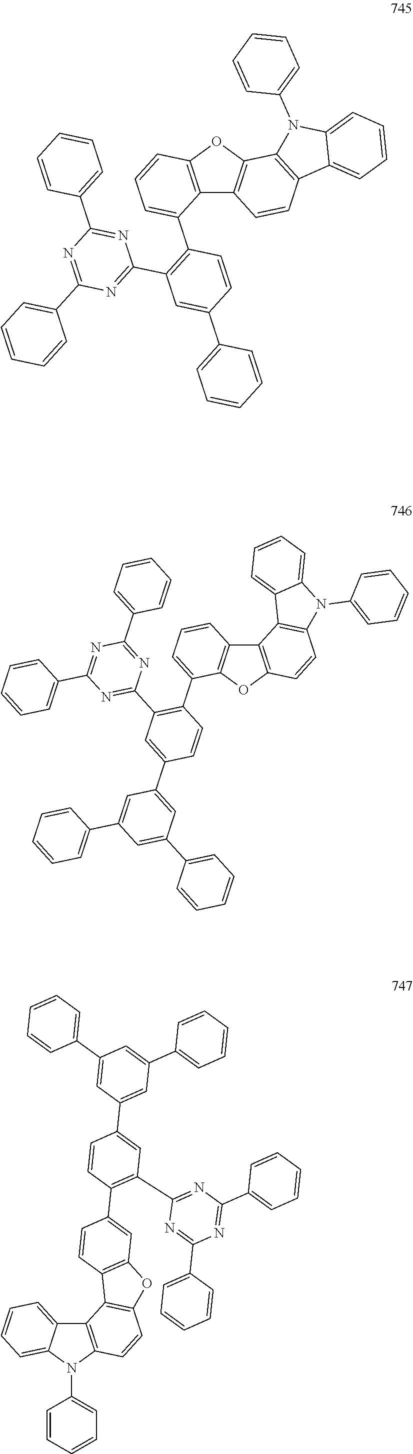 [SMILES: c1ccc(-c2ccc(-c3cccc4oc5c(ccc6c7ccccc7n(-c7ccccc7)c65)c34)c(-c3nc(-c4ccccc4)nc(-c4ccccc4)n3)c2)cc1 ; c1ccc(-c2cc(-c3ccccc3)cc(-c3ccc(-c4cccc5c4oc4ccc6c(c7ccccc7n6-c6ccccc6)c45)c(-c4nc(-c5ccccc5)nc(-c5ccccc5)n4)c3)c2)cc1 ; c1ccc(-c2cc(-c3ccccc3)cc(-c3ccc(-c4ccc5c(c4)oc4ccc6c(c7ccccc7n6-c6ccccc6)c45)c(-c4nc(-c5ccccc5)nc(-c5ccccc5)n4)c3)c2)cc1]

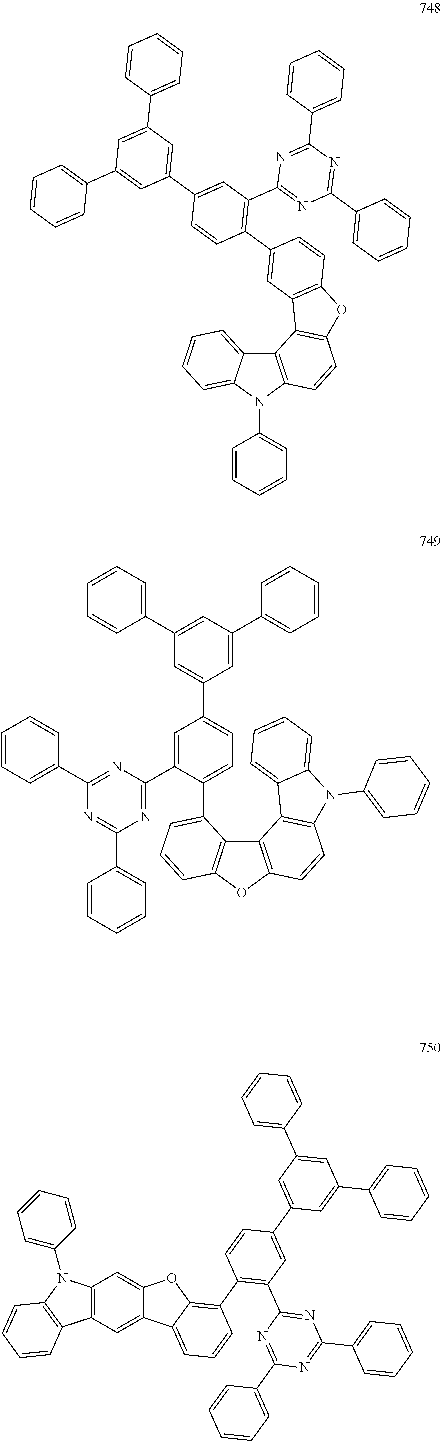 [SMILES: c1ccc(-c2cc(-c3ccccc3)cc(-c3ccc(-c4ccc5oc6ccc7c(c8ccccc8n7-c7ccccc7)c6c5c4)c(-c4nc(-c5ccccc5)nc(-c5ccccc5)n4)c3)c2)cc1 ; c1ccc(-c2cc(-c3ccccc3)cc(-c3ccc(-c4cccc5oc6ccc7c(c8ccccc8n7-c7ccccc7)c6c45)c(-c4nc(-c5ccccc5)nc(-c5ccccc5)n4)c3)c2)cc1 ; c1ccc(-c2cc(-c3ccccc3)cc(-c3ccc(-c4cccc5c4oc4cc6c(cc45)c4ccccc4n6-c4ccccc4)c(-c4nc(-c5ccccc5)nc(-c5ccccc5)n4)c3)c2)cc1]

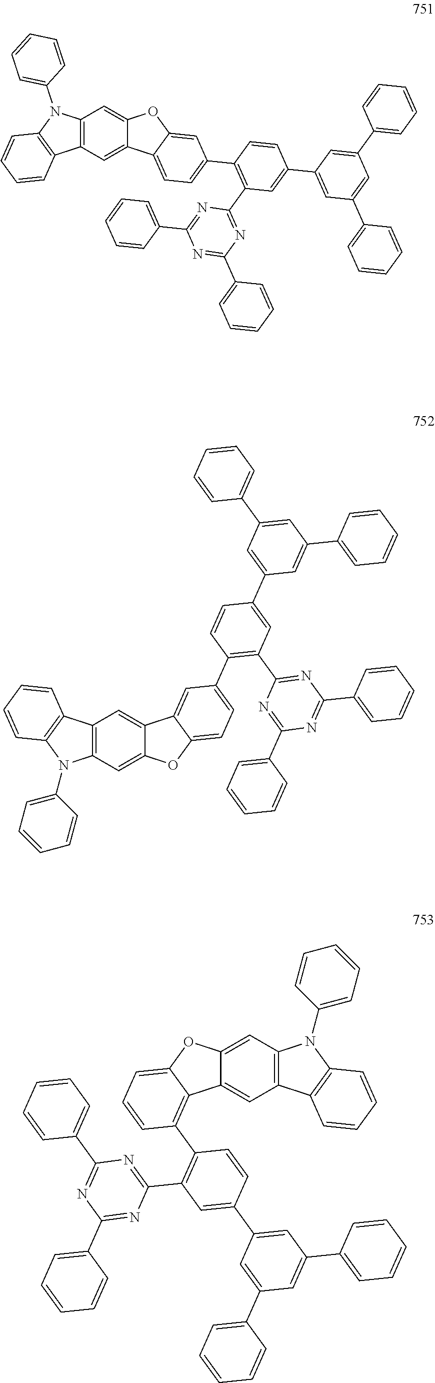 [SMILES: c1ccc(-c2cc(-c3ccccc3)cc(-c3ccc(-c4ccc5c(c4)oc4cc6c(cc45)c4ccccc4n6-c4ccccc4)c(-c4nc(-c5ccccc5)nc(-c5ccccc5)n4)c3)c2)cc1 ; c1ccc(-c2cc(-c3ccccc3)cc(-c3ccc(-c4ccc5oc6cc7c(cc6c5c4)c4ccccc4n7-c4ccccc4)c(-c4nc(-c5ccccc5)nc(-c5ccccc5)n4)c3)c2)cc1 ; c1ccc(-c2cc(-c3ccccc3)cc(-c3ccc(-c4cccc5oc6cc7c(cc6c45)c4ccccc4n7-c4ccccc4)c(-c4nc(-c5ccccc5)nc(-c5ccccc5)n4)c3)c2)cc1]

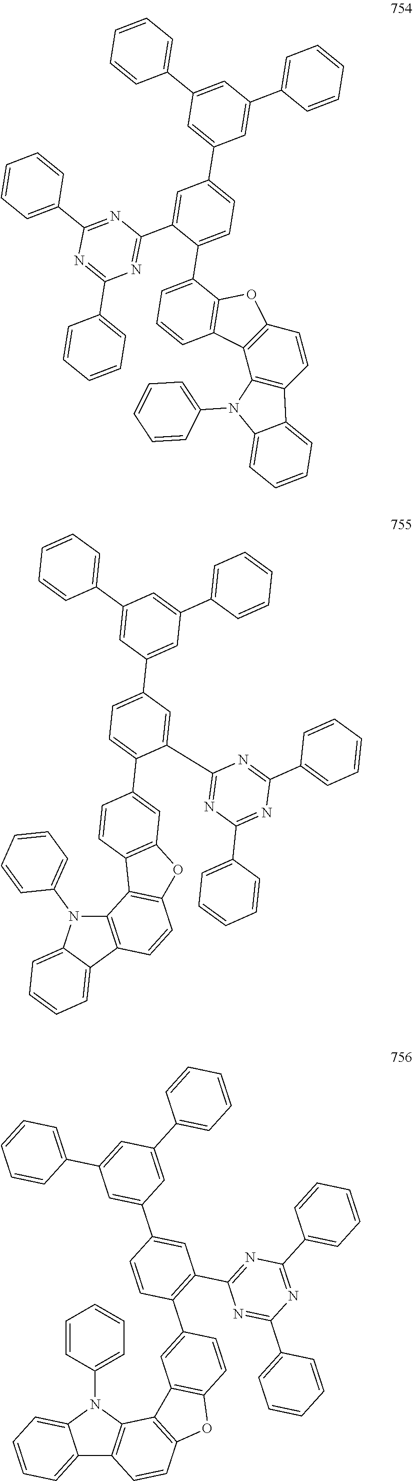 [SMILES: c1ccc(-c2cc(-c3ccccc3)cc(-c3ccc(-c4cccc5c4oc4ccc6c7ccccc7n(-c7ccccc7)c6c45)c(-c4nc(-c5ccccc5)nc(-c5ccccc5)n4)c3)c2)cc1 ; c1ccc(-c2cc(-c3ccccc3)cc(-c3ccc(-c4ccc5c(c4)oc4ccc6c7ccccc7n(-c7ccccc7)c6c45)c(-c4nc(-c5ccccc5)nc(-c5ccccc5)n4)c3)c2)cc1 ; c1ccc(-c2cc(-c3ccccc3)cc(-c3ccc(-c4ccc5oc6ccc7c8ccccc8n(-c8ccccc8)c7c6c5c4)c(-c4nc(-c5ccccc5)nc(-c5ccccc5)n4)c3)c2)cc1]

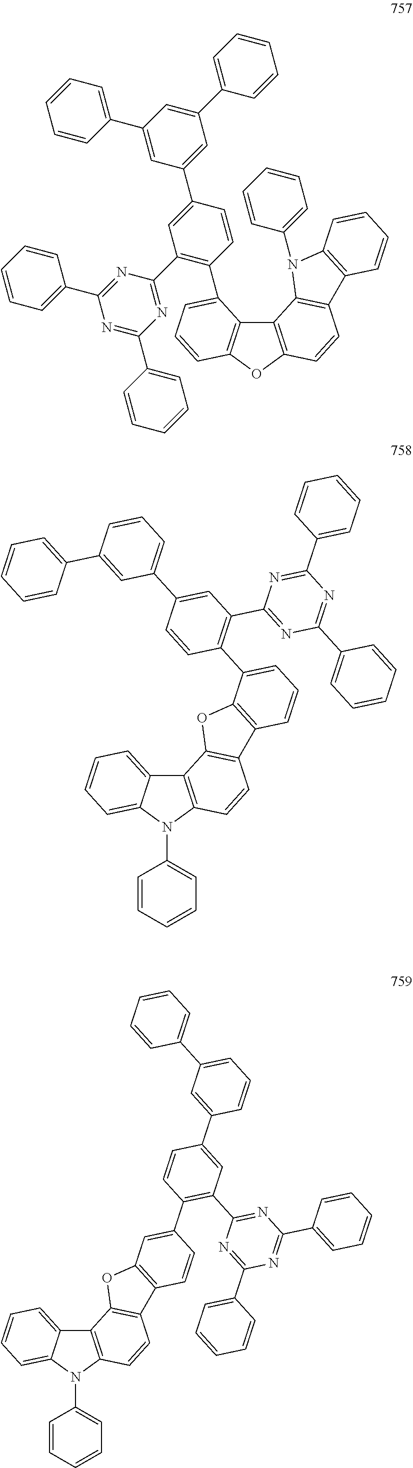 [SMILES: c1ccc(-c2cc(-c3ccccc3)cc(-c3ccc(-c4cccc5oc6ccc7c8ccccc8n(-c8ccccc8)c7c6c45)c(-c4nc(-c5ccccc5)nc(-c5ccccc5)n4)c3)c2)cc1 ; c1ccc(-c2cccc(-c3ccc(-c4cccc5c4oc4c5ccc5c4c4ccccc4n5-c4ccccc4)c(-c4nc(-c5ccccc5)nc(-c5ccccc5)n4)c3)c2)cc1 ; c1ccc(-c2cccc(-c3ccc(-c4ccc5c(c4)oc4c5ccc5c4c4ccccc4n5-c4ccccc4)c(-c4nc(-c5ccccc5)nc(-c5ccccc5)n4)c3)c2)cc1]

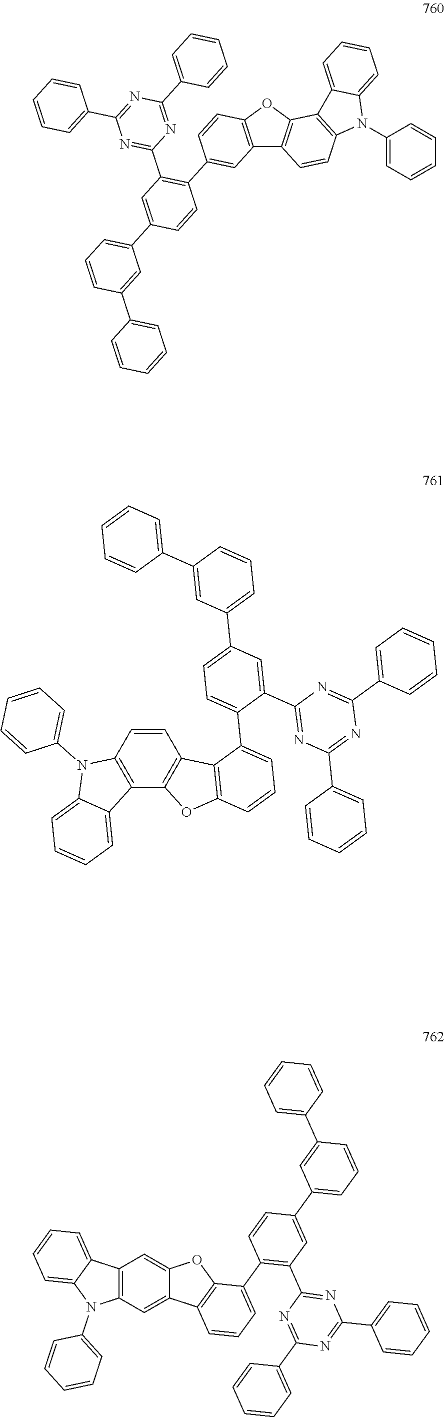 [SMILES: c1ccc(-c2cccc(-c3ccc(-c4ccc5oc6c(ccc7c6c6ccccc6n7-c6ccccc6)c5c4)c(-c4nc(-c5ccccc5)nc(-c5ccccc5)n4)c3)c2)cc1 ; c1ccc(-c2cccc(-c3ccc(-c4cccc5oc6c(ccc7c6c6ccccc6n7-c6ccccc6)c45)c(-c4nc(-c5ccccc5)nc(-c5ccccc5)n4)c3)c2)cc1 ; c1ccc(-c2cccc(-c3ccc(-c4cccc5c4oc4cc6c7ccccc7n(-c7ccccc7)c6cc45)c(-c4nc(-c5ccccc5)nc(-c5ccccc5)n4)c3)c2)cc1]

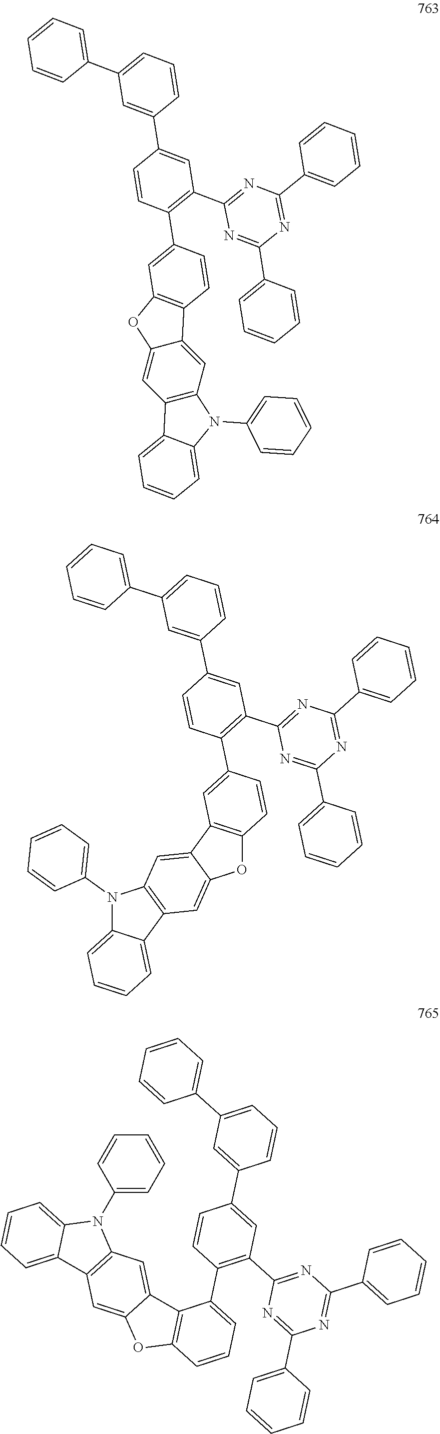 [SMILES: c1ccc(-c2cccc(-c3ccc(-c4ccc5c(c4)oc4cc6c7ccccc7n(-c7ccccc7)c6cc45)c(-c4nc(-c5ccccc5)nc(-c5ccccc5)n4)c3)c2)cc1 ; c1ccc(-c2cccc(-c3ccc(-c4ccc5oc6cc7c8ccccc8n(-c8ccccc8)c7cc6c5c4)c(-c4nc(-c5ccccc5)nc(-c5ccccc5)n4)c3)c2)cc1 ; c1ccc(-c2cccc(-c3ccc(-c4cccc5oc6cc7c8ccccc8n(-c8ccccc8)c7cc6c45)c(-c4nc(-c5ccccc5)nc(-c5ccccc5)n4)c3)c2)cc1]

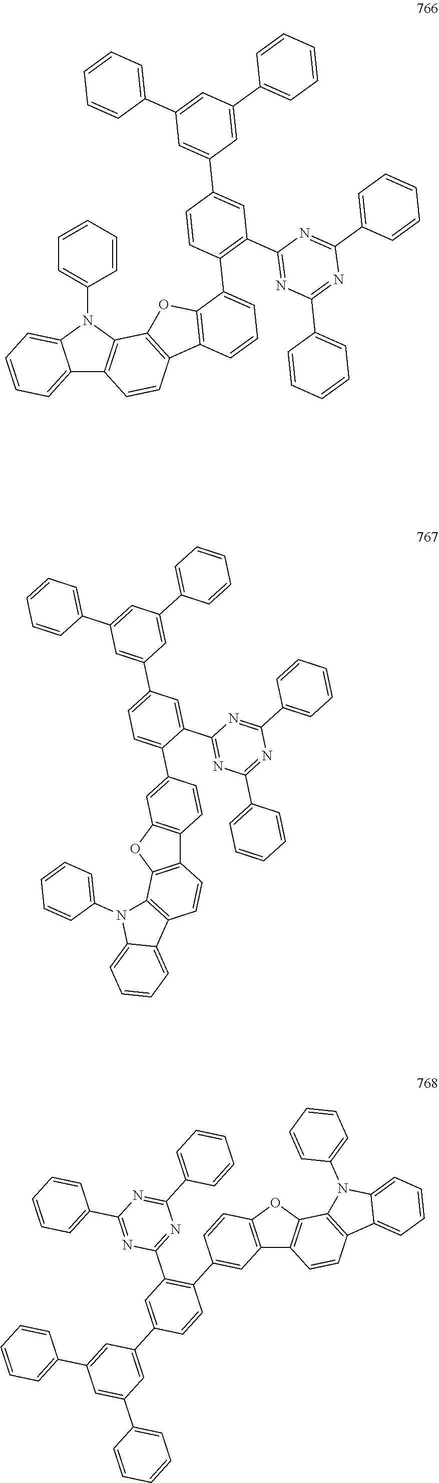 [SMILES: c1ccc(-c2cc(-c3ccccc3)cc(-c3ccc(-c4cccc5c4oc4c5ccc5c6ccccc6n(-c6ccccc6)c54)c(-c4nc(-c5ccccc5)nc(-c5ccccc5)n4)c3)c2)cc1 ; c1ccc(-c2cc(-c3ccccc3)cc(-c3ccc(-c4ccc5c(c4)oc4c5ccc5c6ccccc6n(-c6ccccc6)c54)c(-c4nc(-c5ccccc5)nc(-c5ccccc5)n4)c3)c2)cc1 ; c1ccc(-c2cc(-c3ccccc3)cc(-c3ccc(-c4ccc5oc6c(ccc7c8ccccc8n(-c8ccccc8)c76)c5c4)c(-c4nc(-c5ccccc5)nc(-c5ccccc5)n4)c3)c2)cc1]

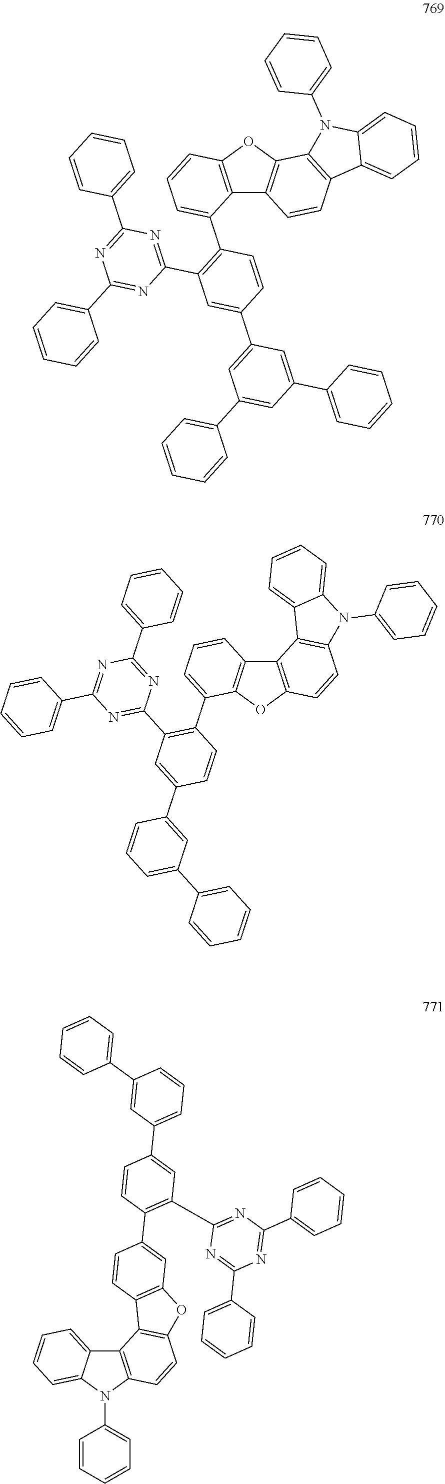 [SMILES: c1ccc(-c2cc(-c3ccccc3)cc(-c3ccc(-c4cccc5oc6c(ccc7c8ccccc8n(-c8ccccc8)c76)c45)c(-c4nc(-c5ccccc5)nc(-c5ccccc5)n4)c3)c2)cc1 ; c1ccc(-c2cccc(-c3ccc(-c4cccc5c4oc4ccc6c(c7ccccc7n6-c6ccccc6)c45)c(-c4nc(-c5ccccc5)nc(-c5ccccc5)n4)c3)c2)cc1 ; c1ccc(-c2cccc(-c3ccc(-c4ccc5c(c4)oc4ccc6c(c7ccccc7n6-c6ccccc6)c45)c(-c4nc(-c5ccccc5)nc(-c5ccccc5)n4)c3)c2)cc1]

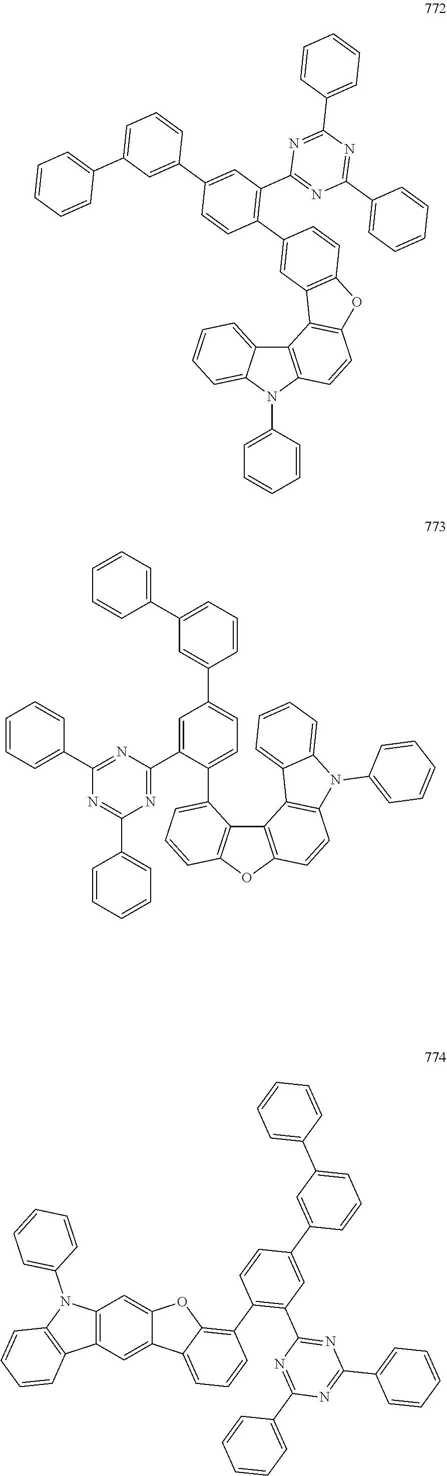 [SMILES: c1ccc(-c2cccc(-c3ccc(-c4ccc5oc6ccc7c(c8ccccc8n7-c7ccccc7)c6c5c4)c(-c4nc(-c5ccccc5)nc(-c5ccccc5)n4)c3)c2)cc1 ; c1ccc(-c2cccc(-c3ccc(-c4cccc5oc6ccc7c(c8ccccc8n7-c7ccccc7)c6c45)c(-c4nc(-c5ccccc5)nc(-c5ccccc5)n4)c3)c2)cc1 ; c1ccc(-c2cccc(-c3ccc(-c4cccc5c4oc4cc6c(cc45)c4ccccc4n6-c4ccccc4)c(-c4nc(-c5ccccc5)nc(-c5ccccc5)n4)c3)c2)cc1]

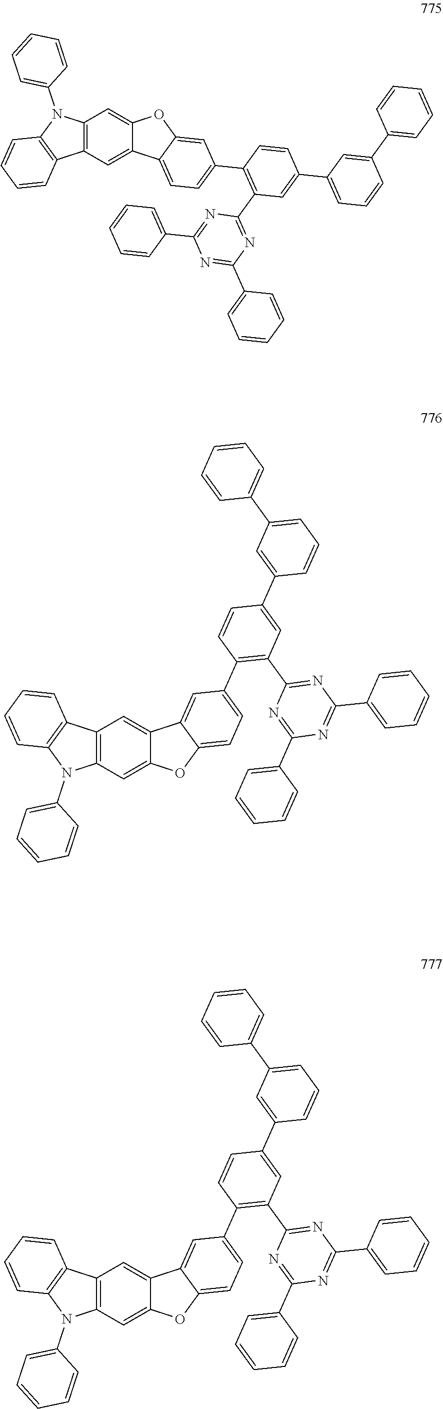 [SMILES: c1ccc(-c2cccc(-c3ccc(-c4ccc5c(c4)oc4cc6c(cc45)c4ccccc4n6-c4ccccc4)c(-c4nc(-c5ccccc5)nc(-c5ccccc5)n4)c3)c2)cc1 ; c1ccc(-c2cccc(-c3ccc(-c4ccc5oc6cc7c(cc6c5c4)c4ccccc4n7-c4ccccc4)c(-c4nc(-c5ccccc5)nc(-c5ccccc5)n4)c3)c2)cc1 ; c1ccc(-c2cccc(-c3ccc(-c4ccc5oc6cc7c(cc6c5c4)c4ccccc4n7-c4ccccc4)c(-c4nc(-c5ccccc5)nc(-c5ccccc5)n4)c3)c2)cc1]

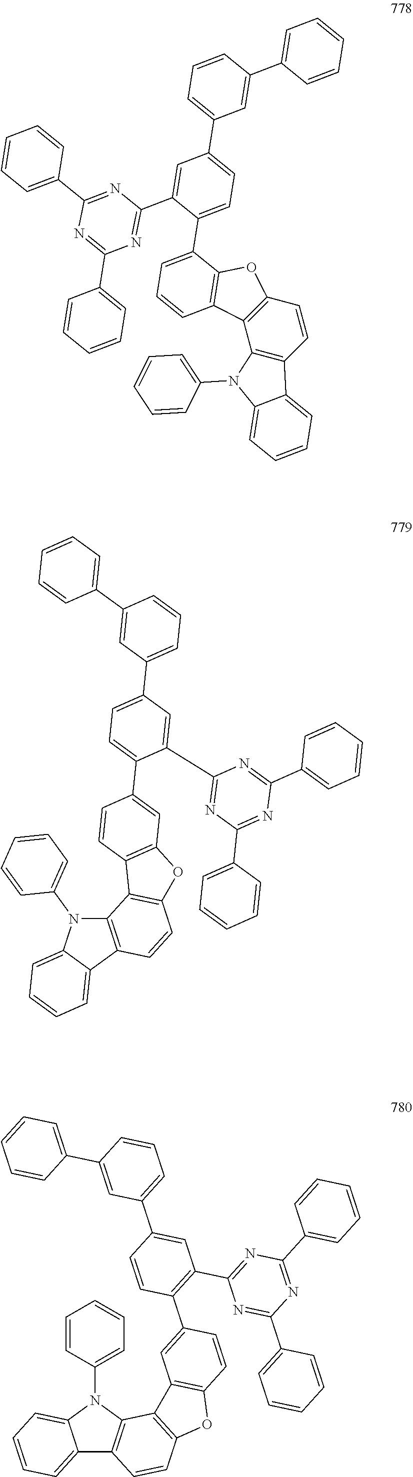 [SMILES: c1ccc(-c2cccc(-c3ccc(-c4cccc5c4oc4ccc6c7ccccc7n(-c7ccccc7)c6c45)c(-c4nc(-c5ccccc5)nc(-c5ccccc5)n4)c3)c2)cc1 ; c1ccc(-c2cccc(-c3ccc(-c4ccc5c(c4)oc4ccc6c7ccccc7n(-c7ccccc7)c6c45)c(-c4nc(-c5ccccc5)nc(-c5ccccc5)n4)c3)c2)cc1 ; c1ccc(-c2cccc(-c3ccc(-c4ccc5oc6ccc7c8ccccc8n(-c8ccccc8)c7c6c5c4)c(-c4nc(-c5ccccc5)nc(-c5ccccc5)n4)c3)c2)cc1]

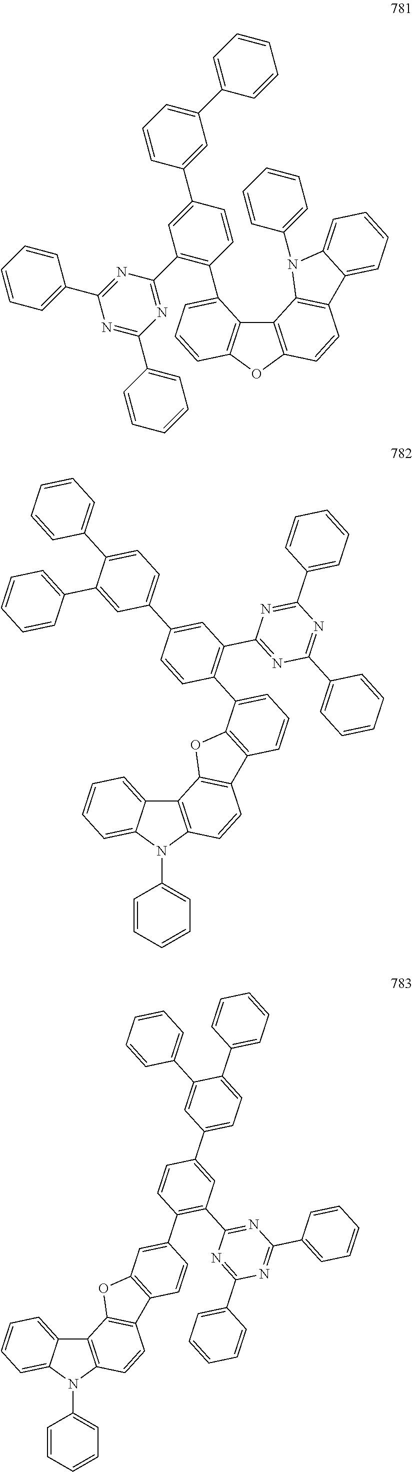 [SMILES: c1ccc(-c2cccc(-c3ccc(-c4cccc5oc6ccc7c8ccccc8n(-c8ccccc8)c7c6c45)c(-c4nc(-c5ccccc5)nc(-c5ccccc5)n4)c3)c2)cc1 ; c1ccc(-c2nc(-c3ccccc3)nc(-c3cc(-c4ccc(-c5ccccc5)c(-c5ccccc5)c4)ccc3-c3cccc4c3oc3c4ccc4c3c3ccccc3n4-c3ccccc3)n2)cc1 ; c1ccc(-c2nc(-c3ccccc3)nc(-c3cc(-c4ccc(-c5ccccc5)c(-c5ccccc5)c4)ccc3-c3ccc4c(c3)oc3c4ccc4c3c3ccccc3n4-c3ccccc3)n2)cc1]

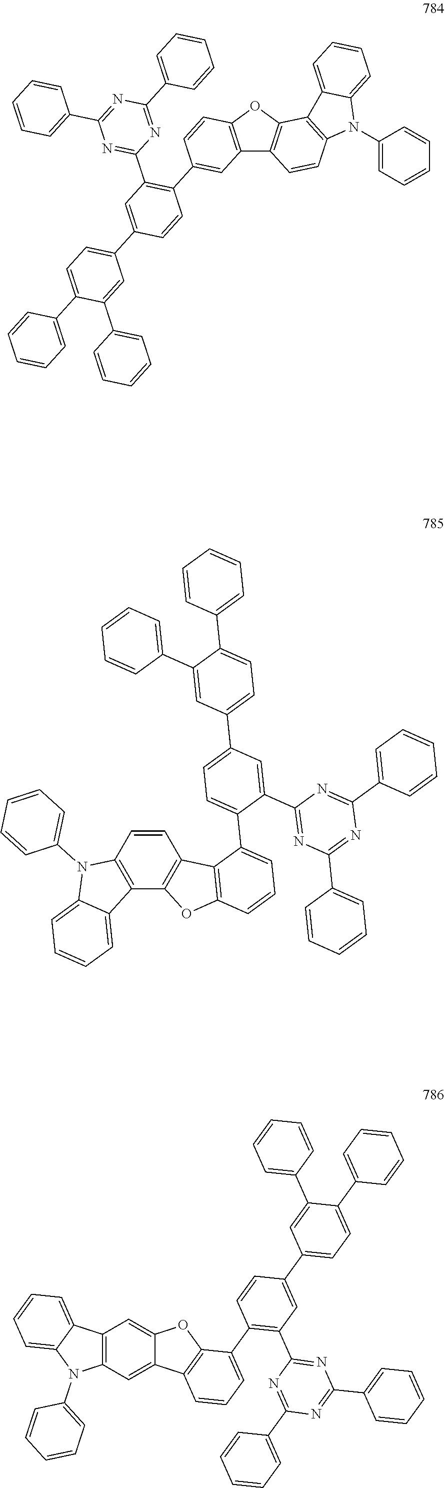 [SMILES: c1ccc(-c2nc(-c3ccccc3)nc(-c3cc(-c4ccc(-c5ccccc5)c(-c5ccccc5)c4)ccc3-c3ccc4oc5c(ccc6c5c5ccccc5n6-c5ccccc5)c4c3)n2)cc1 ; c1ccc(-c2nc(-c3ccccc3)nc(-c3cc(-c4ccc(-c5ccccc5)c(-c5ccccc5)c4)ccc3-c3cccc4oc5c(ccc6c5c5ccccc5n6-c5ccccc5)c34)n2)cc1 ; c1ccc(-c2nc(-c3ccccc3)nc(-c3cc(-c4ccc(-c5ccccc5)c(-c5ccccc5)c4)ccc3-c3cccc4c3oc3cc5c6ccccc6n(-c6ccccc6)c5cc34)n2)cc1]

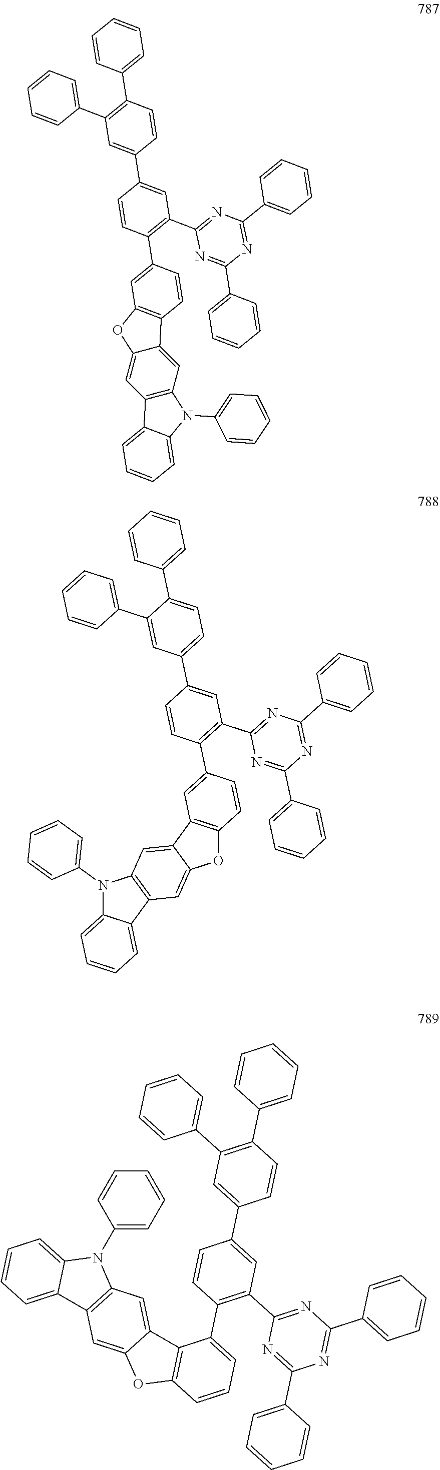

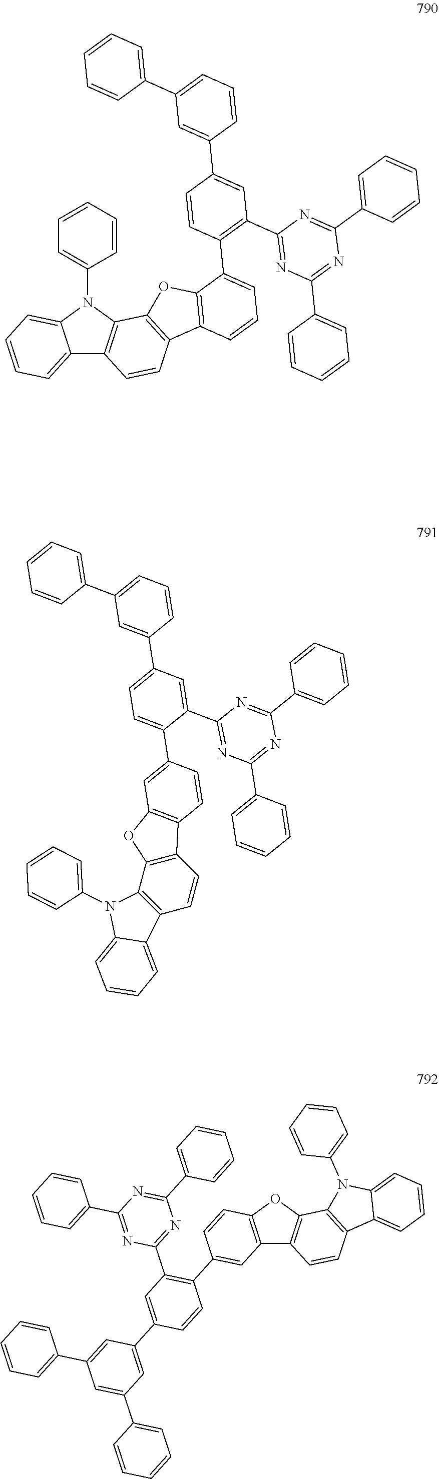 [SMILES: c1ccc(-c2cccc(-c3ccc(-c4cccc5c4oc4c5ccc5c6ccccc6n(-c6ccccc6)c54)c(-c4nc(-c5ccccc5)nc(-c5ccccc5)n4)c3)c2)cc1 ; c1ccc(-c2cccc(-c3ccc(-c4ccc5c(c4)oc4c5ccc5c6ccccc6n(-c6ccccc6)c54)c(-c4nc(-c5ccccc5)nc(-c5ccccc5)n4)c3)c2)cc1 ; c1ccc(-c2cc(-c3ccccc3)cc(-c3ccc(-c4ccc5oc6c(ccc7c8ccccc8n(-c8ccccc8)c76)c5c4)c(-c4nc(-c5ccccc5)nc(-c5ccccc5)n4)c3)c2)cc1]

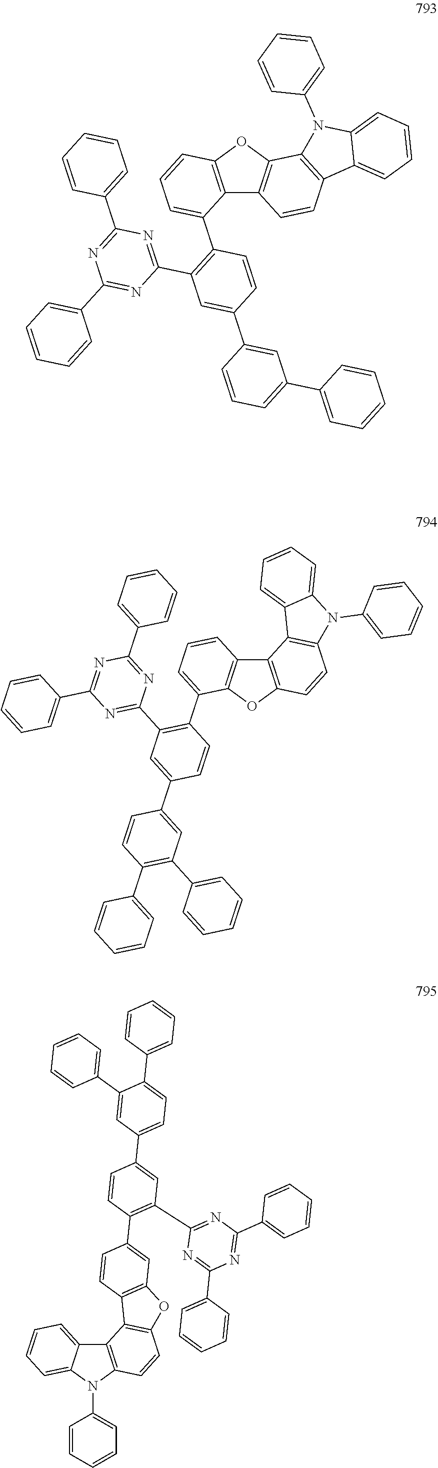 [SMILES: c1ccc(-c2cccc(-c3ccc(-c4cccc5oc6c(ccc7c8ccccc8n(-c8ccccc8)c76)c45)c(-c4nc(-c5ccccc5)nc(-c5ccccc5)n4)c3)c2)cc1 ; c1ccc(-c2nc(-c3ccccc3)nc(-c3cc(-c4ccc(-c5ccccc5)c(-c5ccccc5)c4)ccc3-c3cccc4c3oc3ccc5c(c6ccccc6n5-c5ccccc5)c34)n2)cc1 ; c1ccc(-c2nc(-c3ccccc3)nc(-c3cc(-c4ccc(-c5ccccc5)c(-c5ccccc5)c4)ccc3-c3ccc4c(c3)oc3ccc5c(c6ccccc6n5-c5ccccc5)c34)n2)cc1]

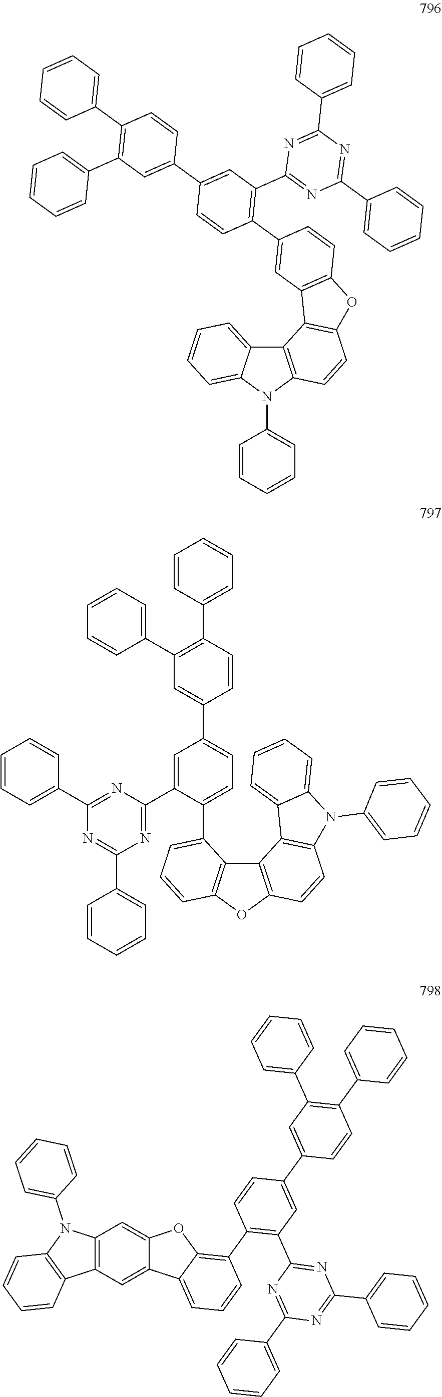 [SMILES: c1ccc(-c2nc(-c3ccccc3)nc(-c3cc(-c4ccc(-c5ccccc5)c(-c5ccccc5)c4)ccc3-c3ccc4oc5ccc6c(c7ccccc7n6-c6ccccc6)c5c4c3)n2)cc1 ; c1ccc(-c2nc(-c3ccccc3)nc(-c3cc(-c4ccc(-c5ccccc5)c(-c5ccccc5)c4)ccc3-c3cccc4oc5ccc6c(c7ccccc7n6-c6ccccc6)c5c34)n2)cc1 ; c1ccc(-c2nc(-c3ccccc3)nc(-c3cc(-c4ccc(-c5ccccc5)c(-c5ccccc5)c4)ccc3-c3cccc4c3oc3cc5c(cc34)c3ccccc3n5-c3ccccc3)n2)cc1]

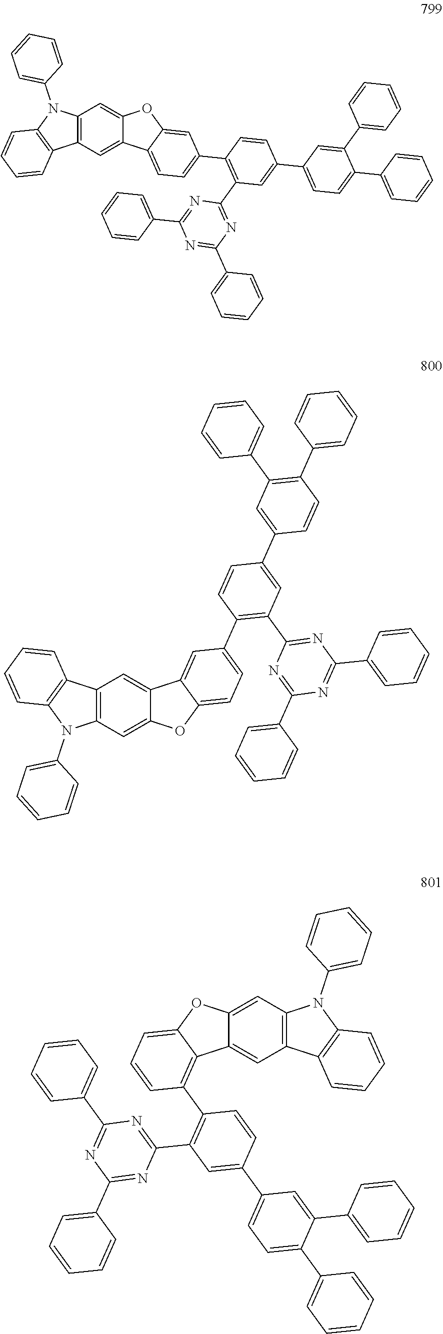 [SMILES: c1ccc(-c2nc(-c3ccccc3)nc(-c3cc(-c4ccc(-c5ccccc5)c(-c5ccccc5)c4)ccc3-c3ccc4c(c3)oc3cc5c(cc34)c3ccccc3n5-c3ccccc3)n2)cc1 ; c1ccc(-c2nc(-c3ccccc3)nc(-c3cc(-c4ccc(-c5ccccc5)c(-c5ccccc5)c4)ccc3-c3ccc4oc5cc6c(cc5c4c3)c3ccccc3n6-c3ccccc3)n2)cc1 ; c1ccc(-c2nc(-c3ccccc3)nc(-c3cc(-c4ccc(-c5ccccc5)c(-c5ccccc5)c4)ccc3-c3cccc4oc5cc6c(cc5c34)c3ccccc3n6-c3ccccc3)n2)cc1]

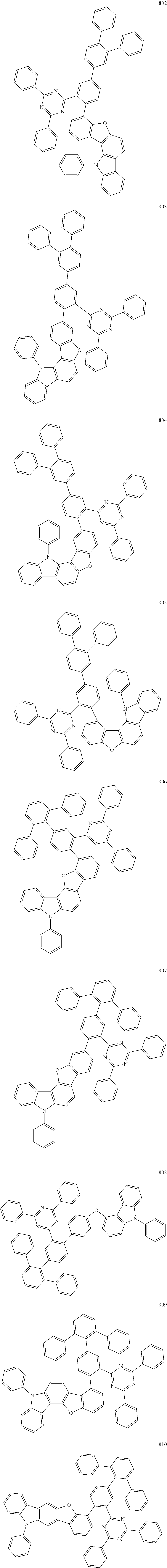

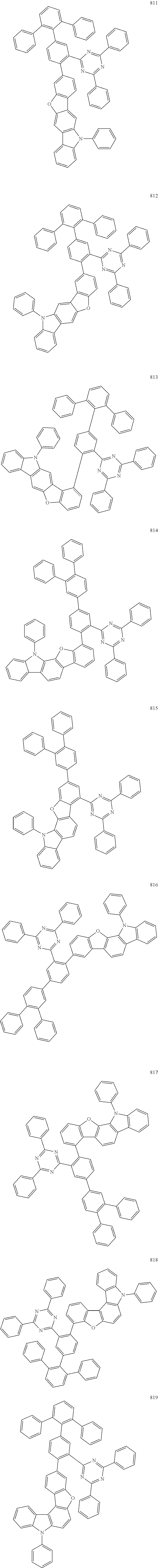

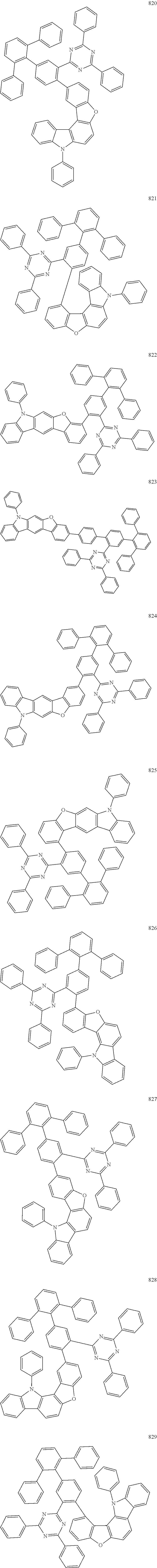

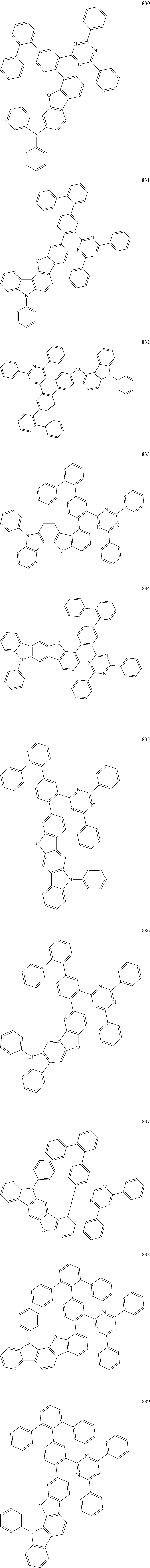

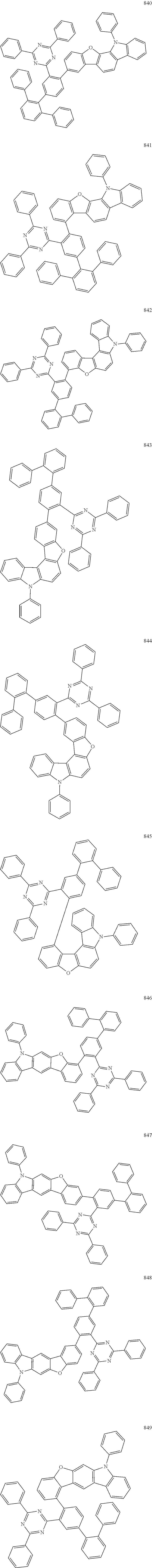

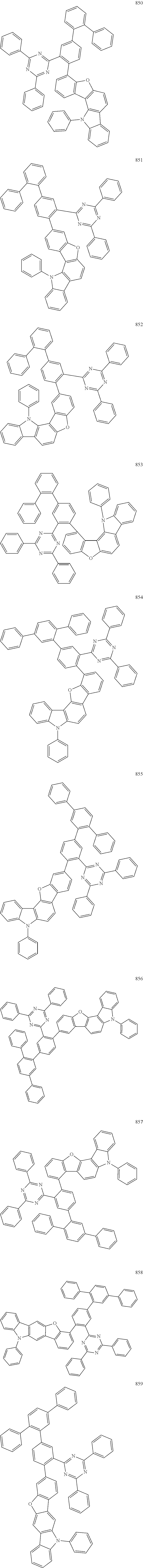

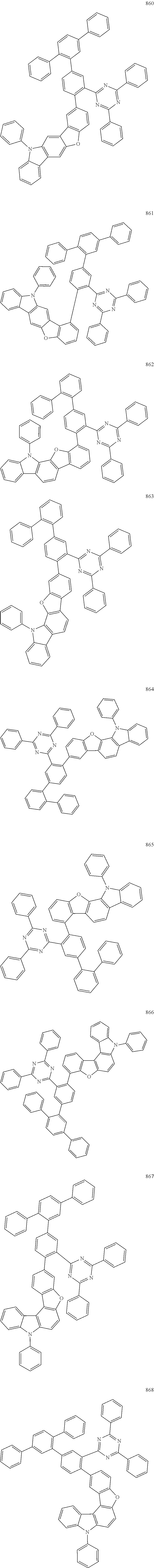

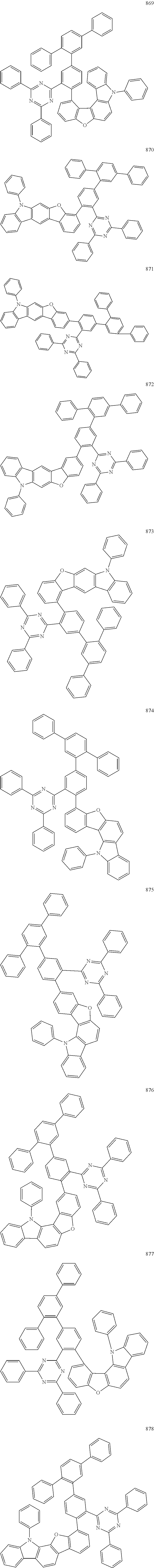

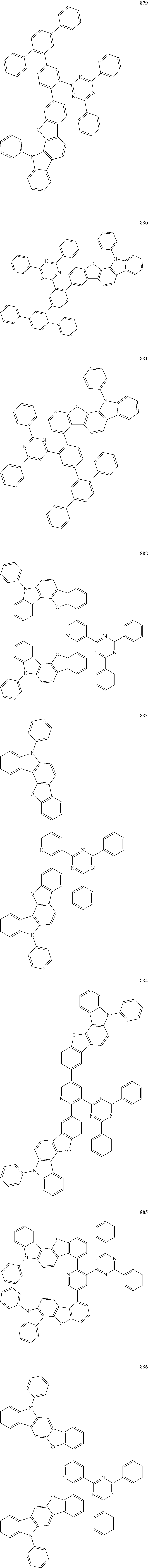

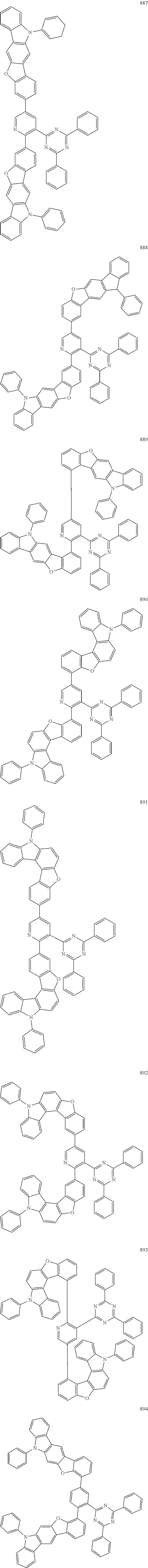

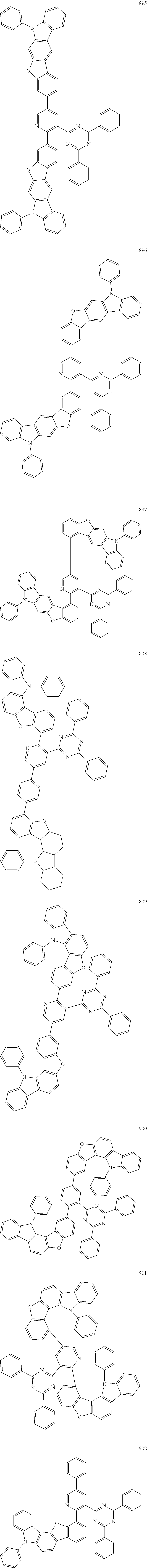

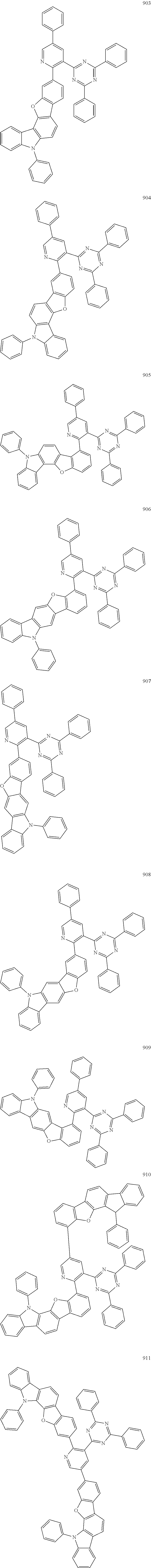

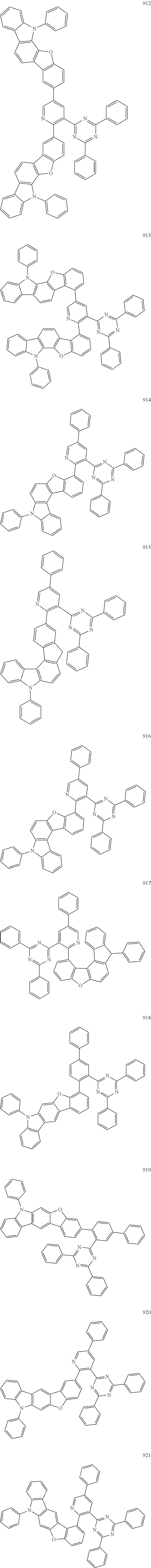

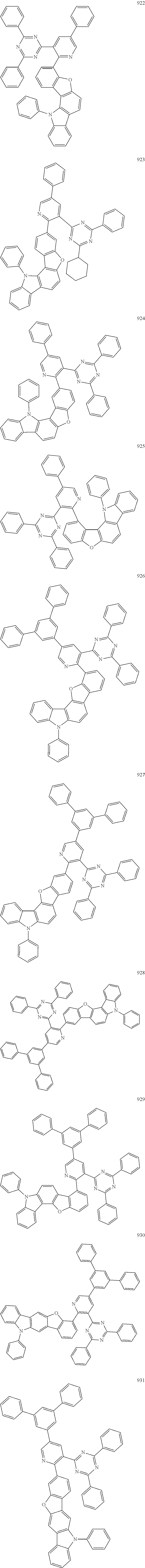

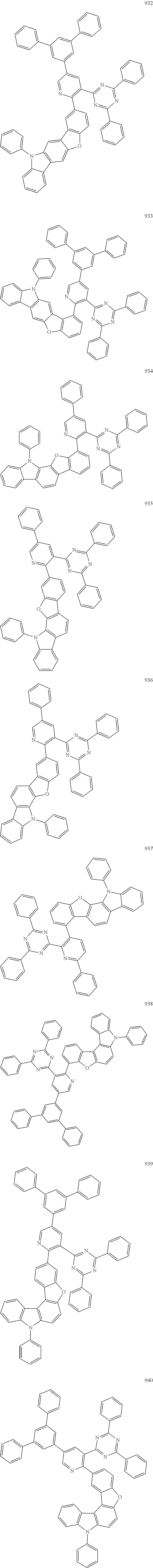

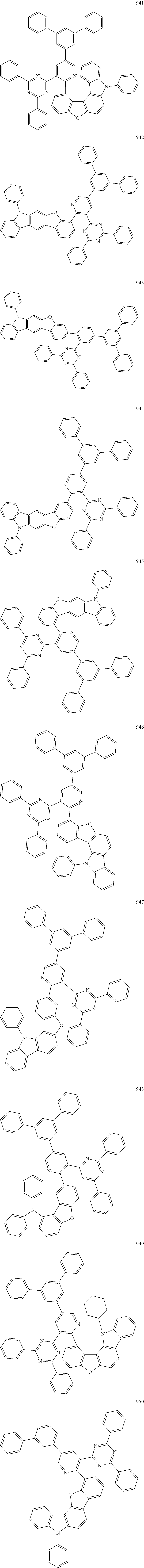

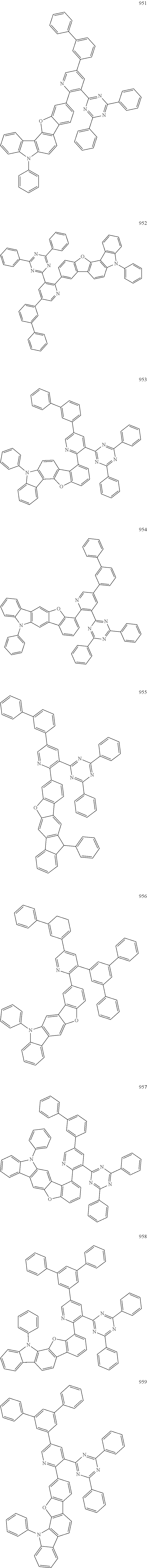

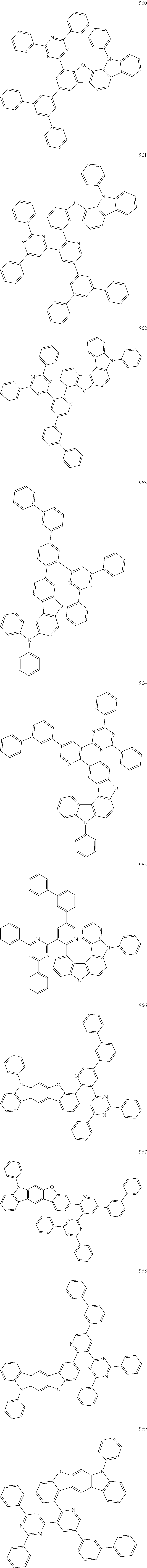

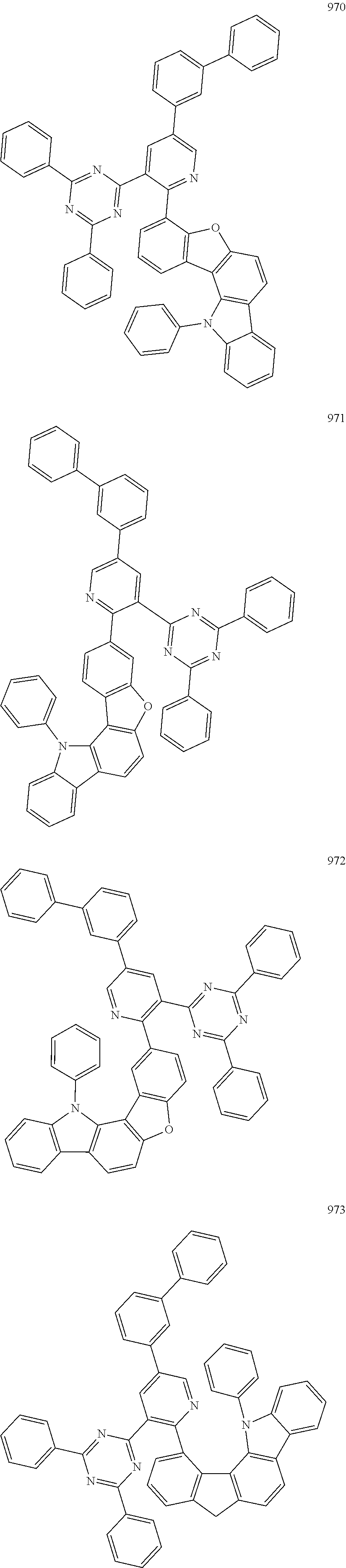 [SMILES: c1ccc(-c2cccc(-c3cnc(-c4cccc5c4oc4ccc6c7ccccc7n(-c7ccccc7)c6c45)c(-c4nc(-c5ccccc5)nc(-c5ccccc5)n4)c3)c2)cc1 ; c1ccc(-c2cccc(-c3cnc(-c4ccc5c(c4)oc4ccc6c7ccccc7n(-c7ccccc7)c6c45)c(-c4nc(-c5ccccc5)nc(-c5ccccc5)n4)c3)c2)cc1 ; c1ccc(-c2cccc(-c3cnc(-c4ccc5oc6ccc7c8ccccc8n(-c8ccccc8)c7c6c5c4)c(-c4nc(-c5ccccc5)nc(-c5ccccc5)n4)c3)c2)cc1 ; c1ccc(-c2cccc(-c3cnc(-c4cccc5c4-c4c(ccc6c7ccccc7n(-c7ccccc7)c46)C5)c(-c4nc(-c5ccccc5)nc(-c5ccccc5)n4)c3)c2)cc1]

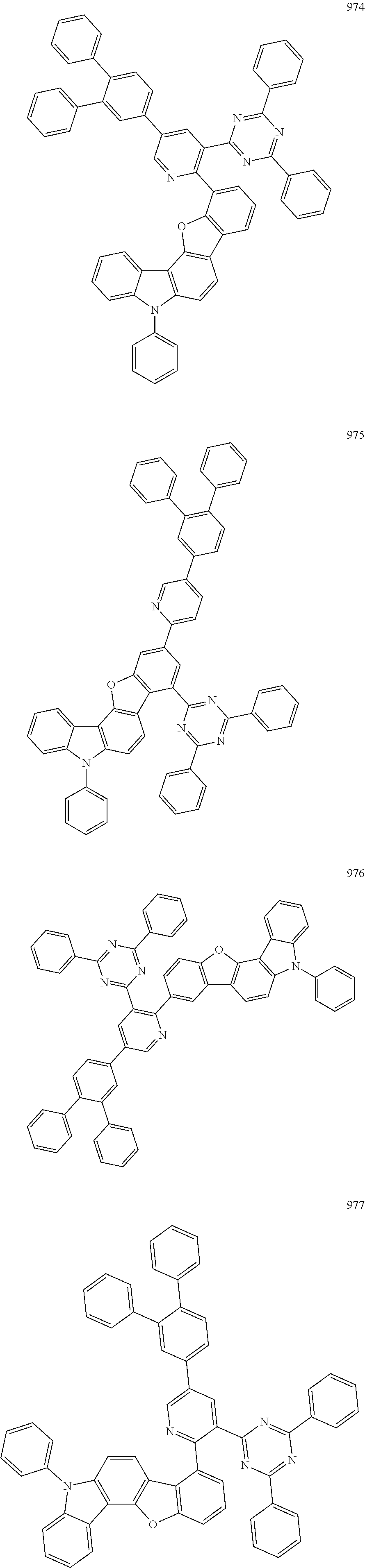 [SMILES: c1ccc(-c2nc(-c3ccccc3)nc(-c3cc(-c4ccc(-c5ccccc5)c(-c5ccccc5)c4)cnc3-c3cccc4c3oc3c4ccc4c3c3ccccc3n4-c3ccccc3)n2)cc1 ; c1ccc(-c2nc(-c3ccccc3)nc(-c3cc(-c4ccc(-c5ccc(-c6ccccc6)c(-c6ccccc6)c5)cn4)cc4oc5c(ccc6c5c5ccccc5n6-c5ccccc5)c34)n2)cc1 ; c1ccc(-c2nc(-c3ccccc3)nc(-c3cc(-c4ccc(-c5ccccc5)c(-c5ccccc5)c4)cnc3-c3ccc4oc5c(ccc6c5c5ccccc5n6-c5ccccc5)c4c3)n2)cc1 ; c1ccc(-c2nc(-c3ccccc3)nc(-c3cc(-c4ccc(-c5ccccc5)c(-c5ccccc5)c4)cnc3-c3cccc4oc5c(ccc6c5c5ccccc5n6-c5ccccc5)c34)n2)cc1]

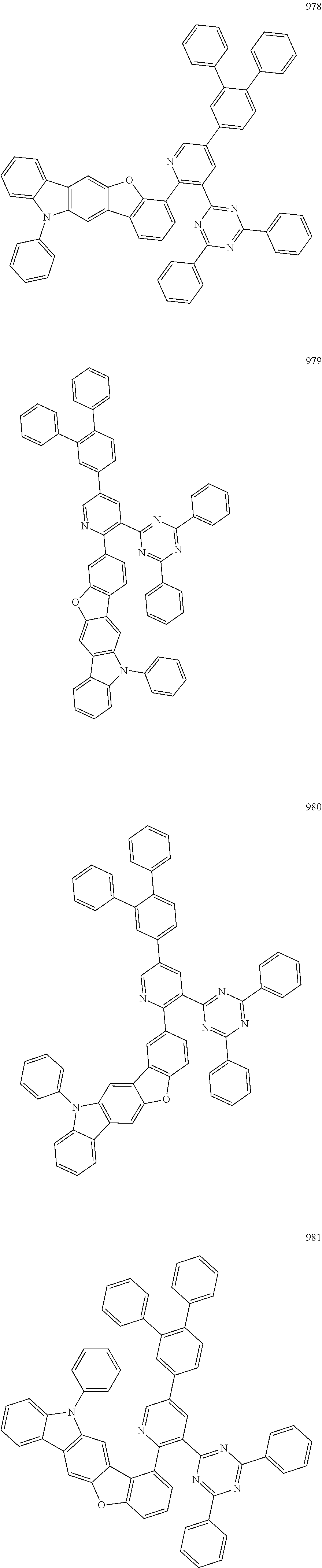 [SMILES: c1ccc(-c2nc(-c3ccccc3)nc(-c3cc(-c4ccc(-c5ccccc5)c(-c5ccccc5)c4)cnc3-c3cccc4c3oc3cc5c6ccccc6n(-c6ccccc6)c5cc34)n2)cc1 ; c1ccc(-c2nc(-c3ccccc3)nc(-c3cc(-c4ccc(-c5ccccc5)c(-c5ccccc5)c4)cnc3-c3ccc4c(c3)oc3cc5c6ccccc6n(-c6ccccc6)c5cc34)n2)cc1 ; c1ccc(-c2nc(-c3ccccc3)nc(-c3cc(-c4ccc(-c5ccccc5)c(-c5ccccc5)c4)cnc3-c3ccc4oc5cc6c7ccccc7n(-c7ccccc7)c6cc5c4c3)n2)cc1 ; c1ccc(-c2nc(-c3ccccc3)nc(-c3cc(-c4ccc(-c5ccccc5)c(-c5ccccc5)c4)cnc3-c3cccc4oc5cc6c7ccccc7n(-c7ccccc7)c6cc5c34)n2)cc1]

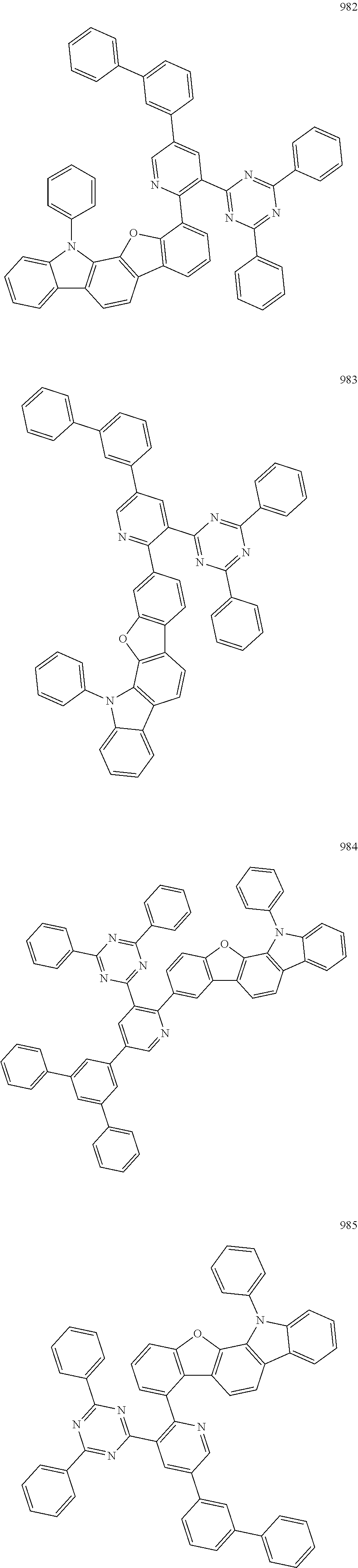 [SMILES: c1ccc(-c2cccc(-c3cnc(-c4cccc5c4oc4c5ccc5c6ccccc6n(-c6ccccc6)c54)c(-c4nc(-c5ccccc5)nc(-c5ccccc5)n4)c3)c2)cc1 ; c1ccc(-c2cccc(-c3cnc(-c4ccc5c(c4)oc4c5ccc5c6ccccc6n(-c6ccccc6)c54)c(-c4nc(-c5ccccc5)nc(-c5ccccc5)n4)c3)c2)cc1 ; c1ccc(-c2cc(-c3ccccc3)cc(-c3cnc(-c4ccc5oc6c(ccc7c8ccccc8n(-c8ccccc8)c76)c5c4)c(-c4nc(-c5ccccc5)nc(-c5ccccc5)n4)c3)c2)cc1 ; c1ccc(-c2cccc(-c3cnc(-c4cccc5oc6c(ccc7c8ccccc8n(-c8ccccc8)c76)c45)c(-c4nc(-c5ccccc5)nc(-c5ccccc5)n4)c3)c2)cc1]

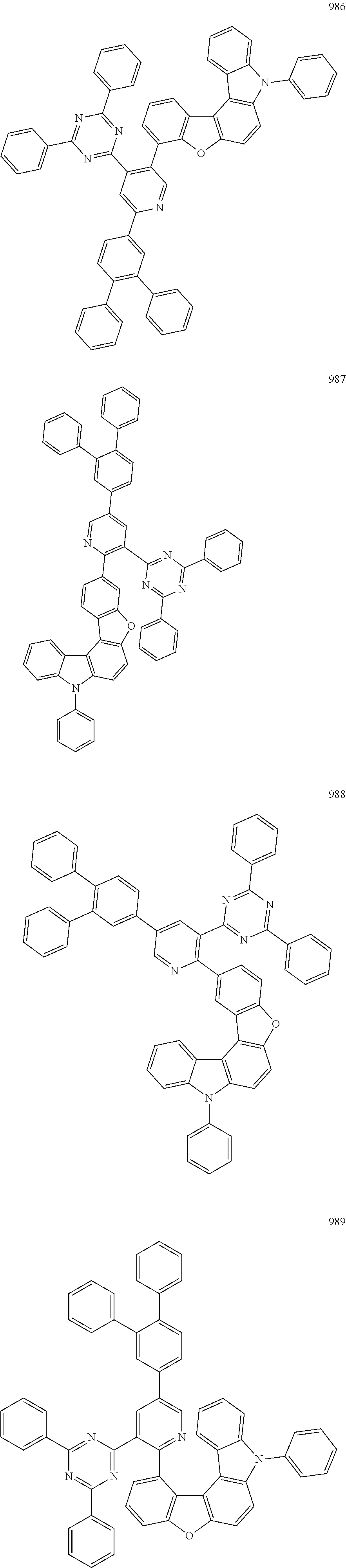 [SMILES: c1ccc(-c2nc(-c3ccccc3)nc(-c3cc(-c4ccc(-c5ccccc5)c(-c5ccccc5)c4)ncc3-c3cccc4c3oc3ccc5c(c6ccccc6n5-c5ccccc5)c34)n2)cc1 ; c1ccc(-c2nc(-c3ccccc3)nc(-c3cc(-c4ccc(-c5ccccc5)c(-c5ccccc5)c4)cnc3-c3ccc4c(c3)oc3ccc5c(c6ccccc6n5-c5ccccc5)c34)n2)cc1 ; c1ccc(-c2nc(-c3ccccc3)nc(-c3cc(-c4ccc(-c5ccccc5)c(-c5ccccc5)c4)cnc3-c3ccc4oc5ccc6c(c7ccccc7n6-c6ccccc6)c5c4c3)n2)cc1 ; c1ccc(-c2nc(-c3ccccc3)nc(-c3cc(-c4ccc(-c5ccccc5)c(-c5ccccc5)c4)cnc3-c3cccc4oc5ccc6c(c7ccccc7n6-c6ccccc6)c5c34)n2)cc1]

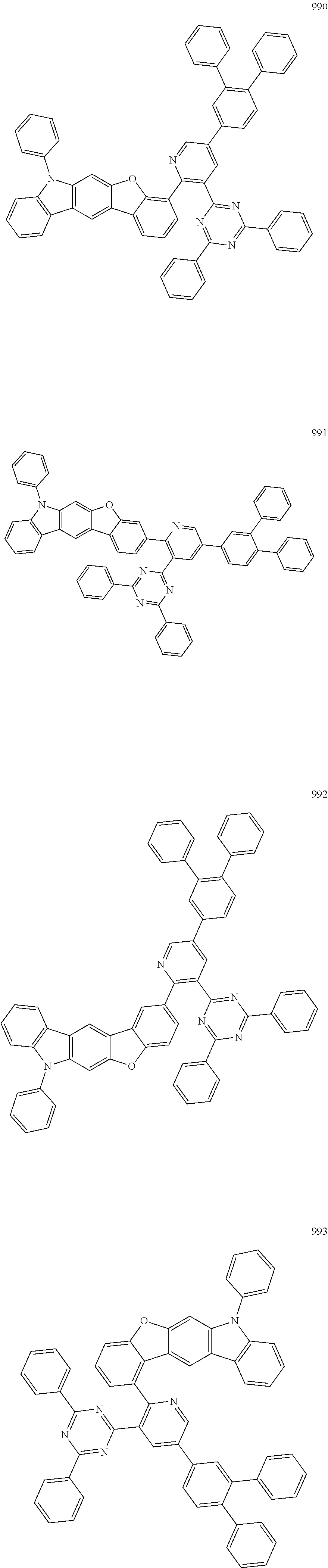 [SMILES: c1ccc(-c2nc(-c3ccccc3)nc(-c3cc(-c4ccc(-c5ccccc5)c(-c5ccccc5)c4)cnc3-c3cccc4c3oc3cc5c(cc34)c3ccccc3n5-c3ccccc3)n2)cc1 ; c1ccc(-c2nc(-c3ccccc3)nc(-c3cc(-c4ccc(-c5ccccc5)c(-c5ccccc5)c4)cnc3-c3ccc4c(c3)oc3cc5c(cc34)c3ccccc3n5-c3ccccc3)n2)cc1 ; c1ccc(-c2nc(-c3ccccc3)nc(-c3cc(-c4ccc(-c5ccccc5)c(-c5ccccc5)c4)cnc3-c3ccc4oc5cc6c(cc5c4c3)c3ccccc3n6-c3ccccc3)n2)cc1 ; c1ccc(-c2nc(-c3ccccc3)nc(-c3cc(-c4ccc(-c5ccccc5)c(-c5ccccc5)c4)cnc3-c3cccc4oc5cc6c(cc5c34)c3ccccc3n6-c3ccccc3)n2)cc1]

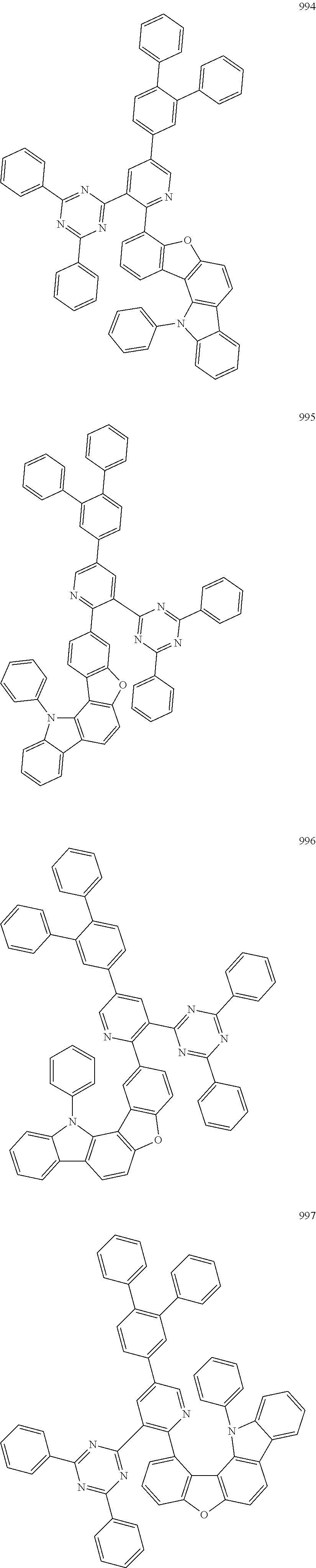 [SMILES: c1ccc(-c2nc(-c3ccccc3)nc(-c3cc(-c4ccc(-c5ccccc5)c(-c5ccccc5)c4)cnc3-c3cccc4c3oc3ccc5c6ccccc6n(-c6ccccc6)c5c34)n2)cc1 ; c1ccc(-c2nc(-c3ccccc3)nc(-c3cc(-c4ccc(-c5ccccc5)c(-c5ccccc5)c4)cnc3-c3ccc4c(c3)oc3ccc5c6ccccc6n(-c6ccccc6)c5c34)n2)cc1 ; c1ccc(-c2nc(-c3ccccc3)nc(-c3cc(-c4ccc(-c5ccccc5)c(-c5ccccc5)c4)cnc3-c3ccc4oc5ccc6c7ccccc7n(-c7ccccc7)c6c5c4c3)n2)cc1 ; c1ccc(-c2nc(-c3ccccc3)nc(-c3cc(-c4ccc(-c5ccccc5)c(-c5ccccc5)c4)cnc3-c3cccc4oc5ccc6c7ccccc7n(-c7ccccc7)c6c5c34)n2)cc1]

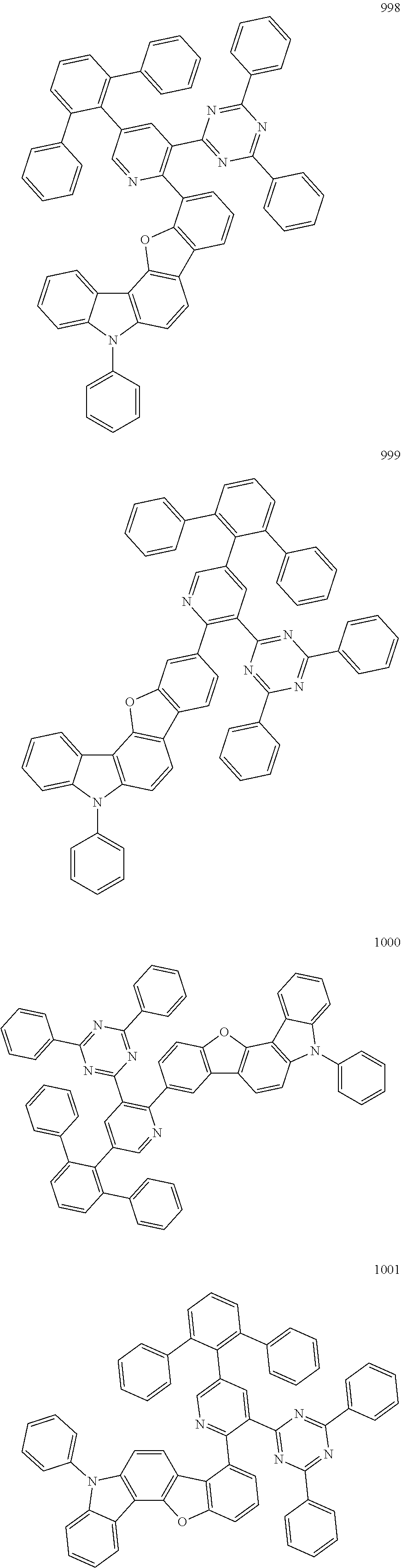 [SMILES: c1ccc(-c2nc(-c3ccccc3)nc(-c3cc(-c4c(-c5ccccc5)cccc4-c4ccccc4)cnc3-c3cccc4c3oc3c4ccc4c3c3ccccc3n4-c3ccccc3)n2)cc1 ; c1ccc(-c2nc(-c3ccccc3)nc(-c3cc(-c4c(-c5ccccc5)cccc4-c4ccccc4)cnc3-c3ccc4c(c3)oc3c4ccc4c3c3ccccc3n4-c3ccccc3)n2)cc1 ; c1ccc(-c2nc(-c3ccccc3)nc(-c3cc(-c4c(-c5ccccc5)cccc4-c4ccccc4)cnc3-c3ccc4oc5c(ccc6c5c5ccccc5n6-c5ccccc5)c4c3)n2)cc1 ; c1ccc(-c2nc(-c3ccccc3)nc(-c3cc(-c4c(-c5ccccc5)cccc4-c4ccccc4)cnc3-c3cccc4oc5c(ccc6c5c5ccccc5n6-c5ccccc5)c34)n2)cc1]

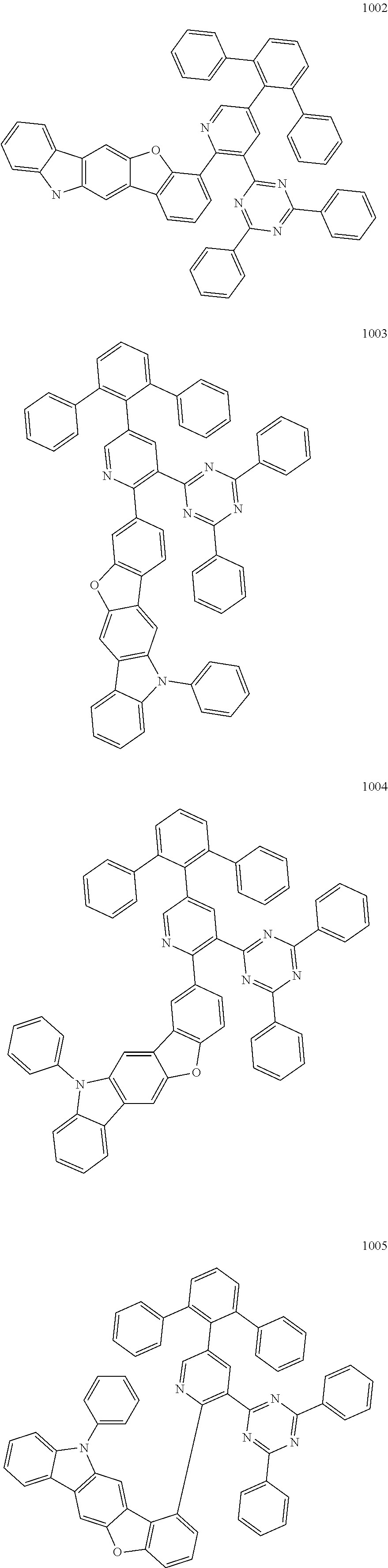 [SMILES: c1ccc(-c2nc(-c3ccccc3)nc(-c3cc(-c4c(-c5ccccc5)cccc4-c4ccccc4)cnc3-c3cccc4c3oc3cc5c(cc34)[nH]c3ccccc35)n2)cc1 ; c1ccc(-c2nc(-c3ccccc3)nc(-c3cc(-c4c(-c5ccccc5)cccc4-c4ccccc4)cnc3-c3ccc4c(c3)oc3cc5c6ccccc6n(-c6ccccc6)c5cc34)n2)cc1 ; c1ccc(-c2nc(-c3ccccc3)nc(-c3cc(-c4c(-c5ccccc5)cccc4-c4ccccc4)cnc3-c3ccc4oc5cc6c7ccccc7n(-c7ccccc7)c6cc5c4c3)n2)cc1 ; c1ccc(-c2nc(-c3ccccc3)nc(-c3cc(-c4c(-c5ccccc5)cccc4-c4ccccc4)cnc3-c3cccc4oc5cc6c7ccccc7n(-c7ccccc7)c6cc5c34)n2)cc1]

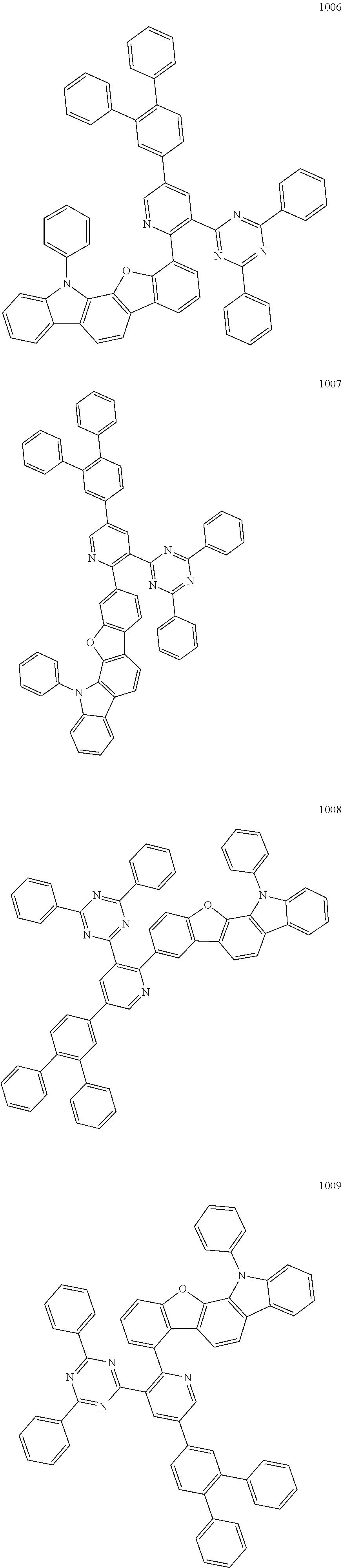 [SMILES: c1ccc(-c2nc(-c3ccccc3)nc(-c3cc(-c4ccc(-c5ccccc5)c(-c5ccccc5)c4)cnc3-c3cccc4c3oc3c4ccc4c5ccccc5n(-c5ccccc5)c43)n2)cc1 ; c1ccc(-c2nc(-c3ccccc3)nc(-c3cc(-c4ccc(-c5ccccc5)c(-c5ccccc5)c4)cnc3-c3ccc4c(c3)oc3c4ccc4c5ccccc5n(-c5ccccc5)c43)n2)cc1 ; c1ccc(-c2nc(-c3ccccc3)nc(-c3cc(-c4ccc(-c5ccccc5)c(-c5ccccc5)c4)cnc3-c3ccc4oc5c(ccc6c7ccccc7n(-c7ccccc7)c65)c4c3)n2)cc1 ; c1ccc(-c2nc(-c3ccccc3)nc(-c3cc(-c4ccc(-c5ccccc5)c(-c5ccccc5)c4)cnc3-c3cccc4oc5c(ccc6c7ccccc7n(-c7ccccc7)c65)c34)n2)cc1]

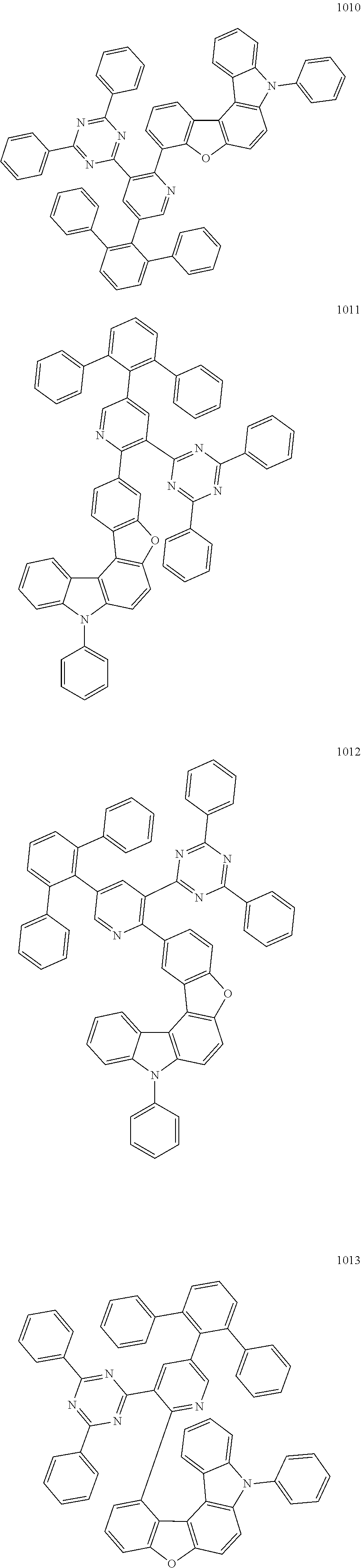

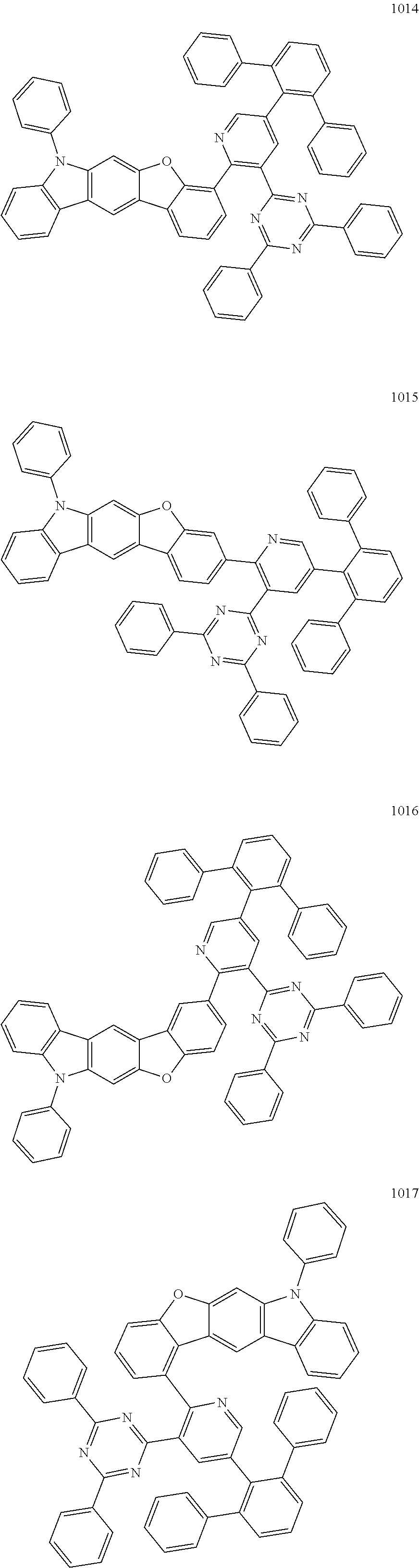 [SMILES: c1ccc(-c2nc(-c3ccccc3)nc(-c3cc(-c4c(-c5ccccc5)cccc4-c4ccccc4)cnc3-c3cccc4c3oc3cc5c(cc34)c3ccccc3n5-c3ccccc3)n2)cc1 ; c1ccc(-c2nc(-c3ccccc3)nc(-c3cc(-c4c(-c5ccccc5)cccc4-c4ccccc4)cnc3-c3ccc4c(c3)oc3cc5c(cc34)c3ccccc3n5-c3ccccc3)n2)cc1 ; c1ccc(-c2nc(-c3ccccc3)nc(-c3cc(-c4c(-c5ccccc5)cccc4-c4ccccc4)cnc3-c3ccc4oc5cc6c(cc5c4c3)c3ccccc3n6-c3ccccc3)n2)cc1 ; c1ccc(-c2nc(-c3ccccc3)nc(-c3cc(-c4c(-c5ccccc5)cccc4-c4ccccc4)cnc3-c3cccc4oc5cc6c(cc5c34)c3ccccc3n6-c3ccccc3)n2)cc1]

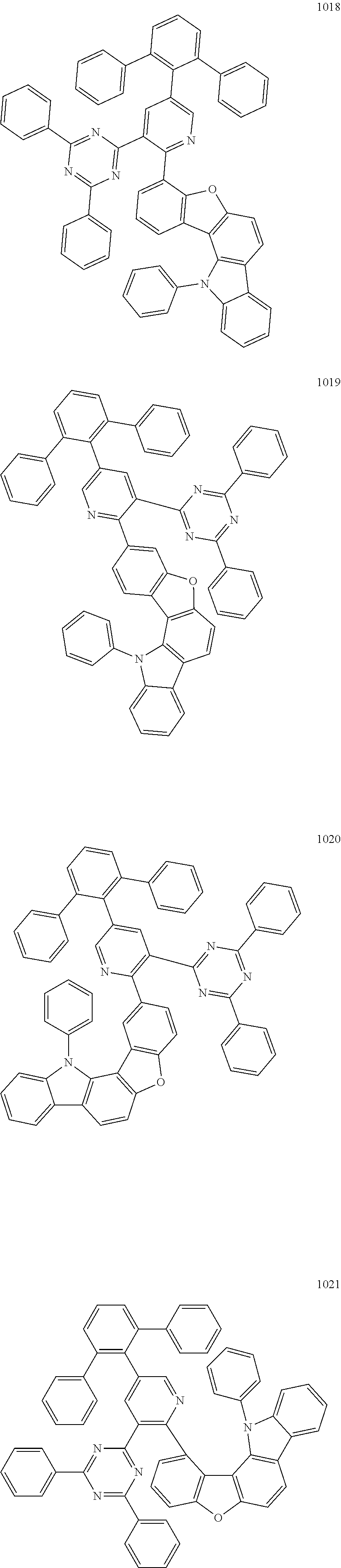 [SMILES: c1ccc(-c2nc(-c3ccccc3)nc(-c3cc(-c4c(-c5ccccc5)cccc4-c4ccccc4)cnc3-c3cccc4c3oc3ccc5c6ccccc6n(-c6ccccc6)c5c34)n2)cc1 ; c1ccc(-c2nc(-c3ccccc3)nc(-c3cc(-c4c(-c5ccccc5)cccc4-c4ccccc4)cnc3-c3ccc4c(c3)oc3ccc5c6ccccc6n(-c6ccccc6)c5c34)n2)cc1 ; c1ccc(-c2nc(-c3ccccc3)nc(-c3cc(-c4c(-c5ccccc5)cccc4-c4ccccc4)cnc3-c3ccc4oc5ccc6c7ccccc7n(-c7ccccc7)c6c5c4c3)n2)cc1 ; c1ccc(-c2nc(-c3ccccc3)nc(-c3cc(-c4c(-c5ccccc5)cccc4-c4ccccc4)cnc3-c3cccc4oc5ccc6c7ccccc7n(-c7ccccc7)c6c5c34)n2)cc1]

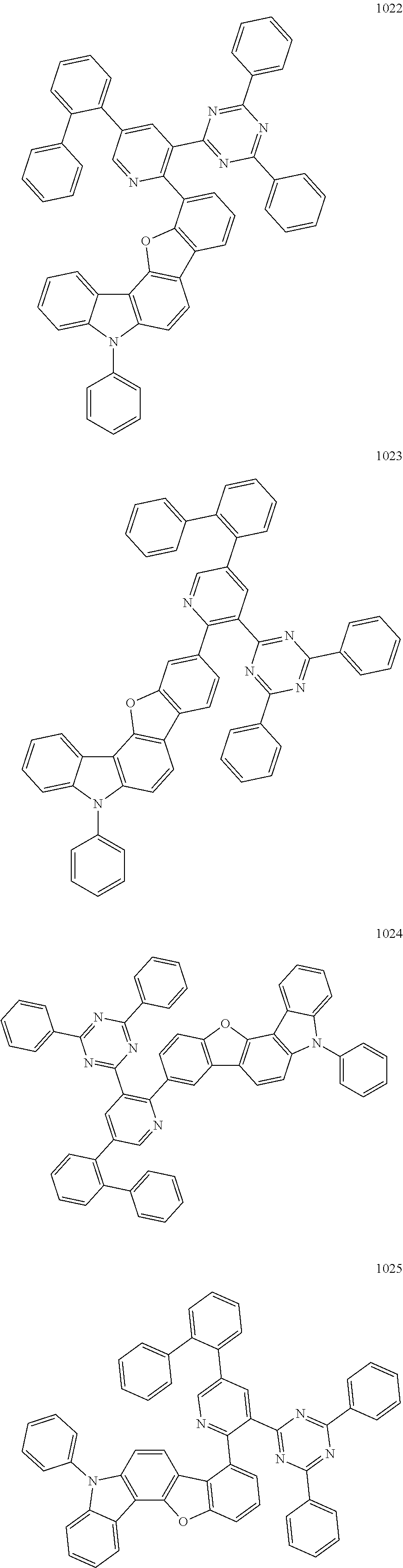 [SMILES: c1ccc(-c2nc(-c3ccccc3)nc(-c3cc(-c4ccccc4-c4ccccc4)cnc3-c3cccc4c3oc3c4ccc4c3c3ccccc3n4-c3ccccc3)n2)cc1 ; c1ccc(-c2nc(-c3ccccc3)nc(-c3cc(-c4ccccc4-c4ccccc4)cnc3-c3ccc4c(c3)oc3c4ccc4c3c3ccccc3n4-c3ccccc3)n2)cc1 ; c1ccc(-c2nc(-c3ccccc3)nc(-c3cc(-c4ccccc4-c4ccccc4)cnc3-c3ccc4oc5c(ccc6c5c5ccccc5n6-c5ccccc5)c4c3)n2)cc1 ; c1ccc(-c2nc(-c3ccccc3)nc(-c3cc(-c4ccccc4-c4ccccc4)cnc3-c3cccc4oc5c(ccc6c5c5ccccc5n6-c5ccccc5)c34)n2)cc1]

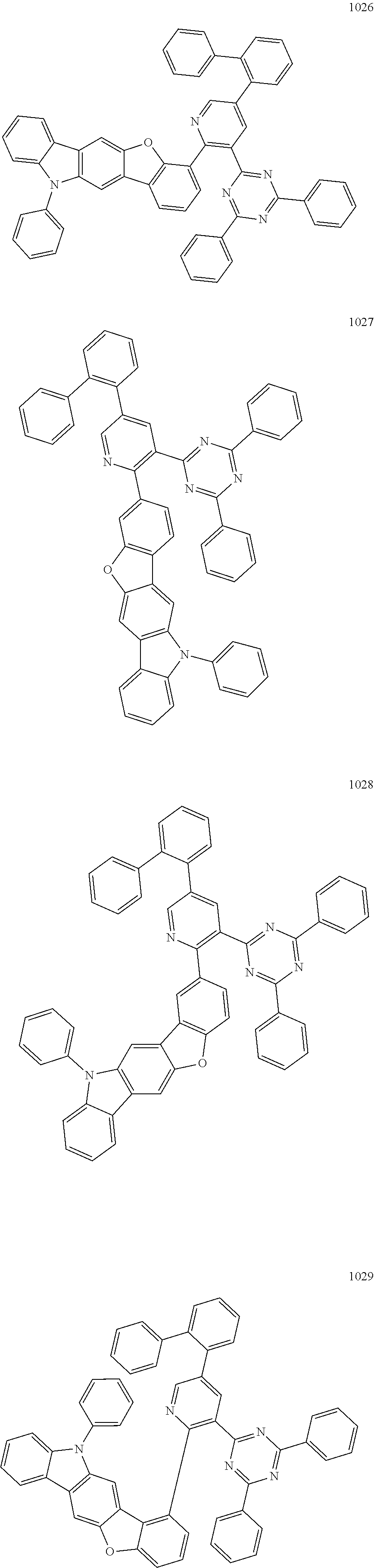 [SMILES: c1ccc(-c2nc(-c3ccccc3)nc(-c3cc(-c4ccccc4-c4ccccc4)cnc3-c3cccc4c3oc3cc5c6ccccc6n(-c6ccccc6)c5cc34)n2)cc1 ; c1ccc(-c2nc(-c3ccccc3)nc(-c3cc(-c4ccccc4-c4ccccc4)cnc3-c3ccc4c(c3)oc3cc5c6ccccc6n(-c6ccccc6)c5cc34)n2)cc1 ; c1ccc(-c2nc(-c3ccccc3)nc(-c3cc(-c4ccccc4-c4ccccc4)cnc3-c3ccc4oc5cc6c7ccccc7n(-c7ccccc7)c6cc5c4c3)n2)cc1 ; c1ccc(-c2nc(-c3ccccc3)nc(-c3cc(-c4ccccc4-c4ccccc4)cnc3-c3cccc4oc5cc6c7ccccc7n(-c7ccccc7)c6cc5c34)n2)cc1]

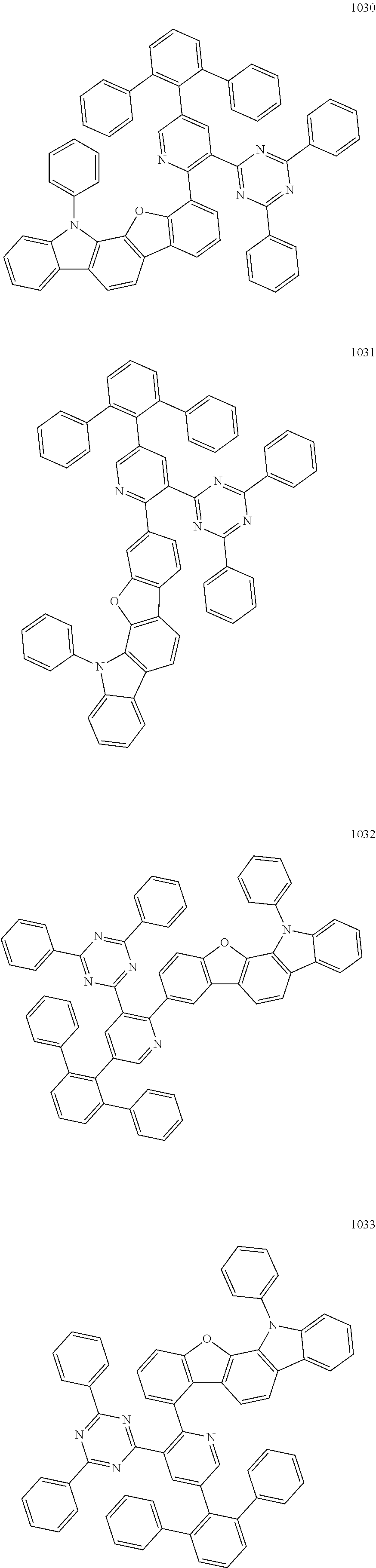 [SMILES: c1ccc(-c2nc(-c3ccccc3)nc(-c3cc(-c4c(-c5ccccc5)cccc4-c4ccccc4)cnc3-c3cccc4c3oc3c4ccc4c5ccccc5n(-c5ccccc5)c43)n2)cc1 ; c1ccc(-c2nc(-c3ccccc3)nc(-c3cc(-c4c(-c5ccccc5)cccc4-c4ccccc4)cnc3-c3ccc4c(c3)oc3c4ccc4c5ccccc5n(-c5ccccc5)c43)n2)cc1 ; c1ccc(-c2nc(-c3ccccc3)nc(-c3cc(-c4c(-c5ccccc5)cccc4-c4ccccc4)cnc3-c3ccc4oc5c(ccc6c7ccccc7n(-c7ccccc7)c65)c4c3)n2)cc1 ; c1ccc(-c2nc(-c3ccccc3)nc(-c3cc(-c4c(-c5ccccc5)cccc4-c4ccccc4)cnc3-c3cccc4oc5c(ccc6c7ccccc7n(-c7ccccc7)c65)c34)n2)cc1]

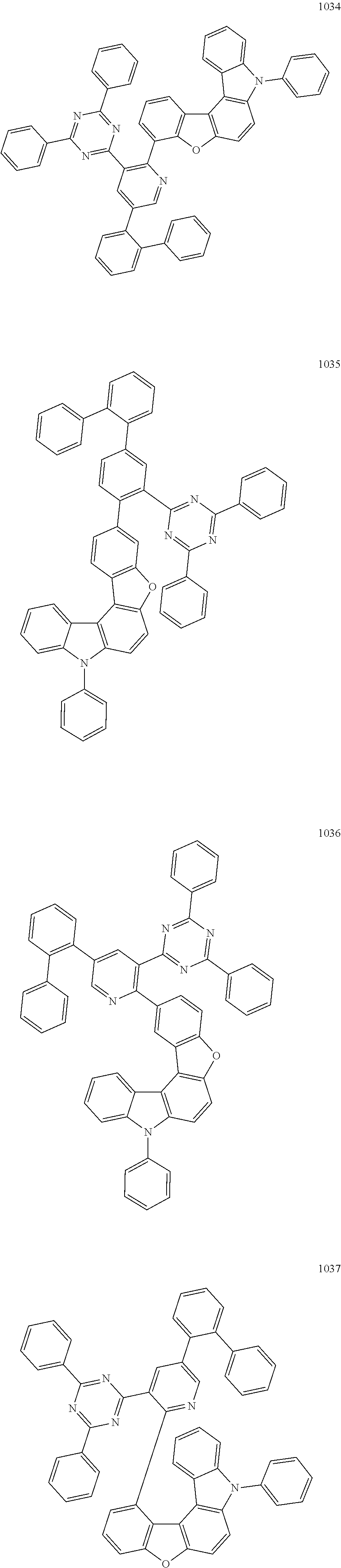 [SMILES: c1ccc(-c2nc(-c3ccccc3)nc(-c3cc(-c4ccccc4-c4ccccc4)cnc3-c3cccc4c3oc3ccc5c(c6ccccc6n5-c5ccccc5)c34)n2)cc1 ; c1ccc(-c2nc(-c3ccccc3)nc(-c3cc(-c4ccccc4-c4ccccc4)ccc3-c3ccc4c(c3)oc3ccc5c(c6ccccc6n5-c5ccccc5)c34)n2)cc1 ; c1ccc(-c2nc(-c3ccccc3)nc(-c3cc(-c4ccccc4-c4ccccc4)cnc3-c3ccc4oc5ccc6c(c7ccccc7n6-c6ccccc6)c5c4c3)n2)cc1 ; c1ccc(-c2nc(-c3ccccc3)nc(-c3cc(-c4ccccc4-c4ccccc4)cnc3-c3cccc4oc5ccc6c(c7ccccc7n6-c6ccccc6)c5c34)n2)cc1]

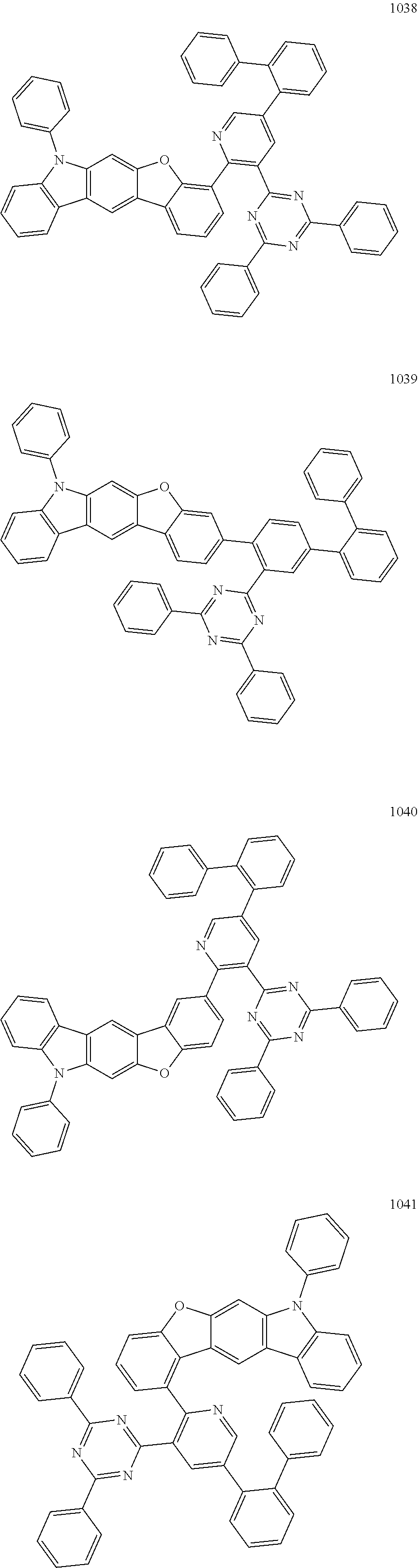 [SMILES: c1ccc(-c2nc(-c3ccccc3)nc(-c3cc(-c4ccccc4-c4ccccc4)cnc3-c3cccc4c3oc3cc5c(cc34)c3ccccc3n5-c3ccccc3)n2)cc1 ; c1ccc(-c2nc(-c3ccccc3)nc(-c3cc(-c4ccccc4-c4ccccc4)ccc3-c3ccc4c(c3)oc3cc5c(cc34)c3ccccc3n5-c3ccccc3)n2)cc1 ; c1ccc(-c2nc(-c3ccccc3)nc(-c3cc(-c4ccccc4-c4ccccc4)cnc3-c3ccc4oc5cc6c(cc5c4c3)c3ccccc3n6-c3ccccc3)n2)cc1 ; c1ccc(-c2nc(-c3ccccc3)nc(-c3cc(-c4ccccc4-c4ccccc4)cnc3-c3cccc4oc5cc6c(cc5c34)c3ccccc3n6-c3ccccc3)n2)cc1]

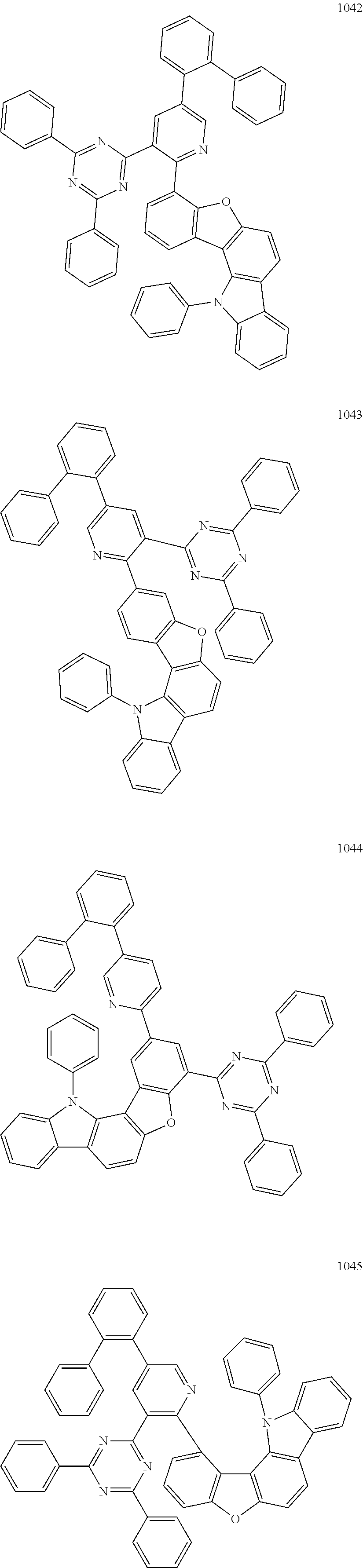

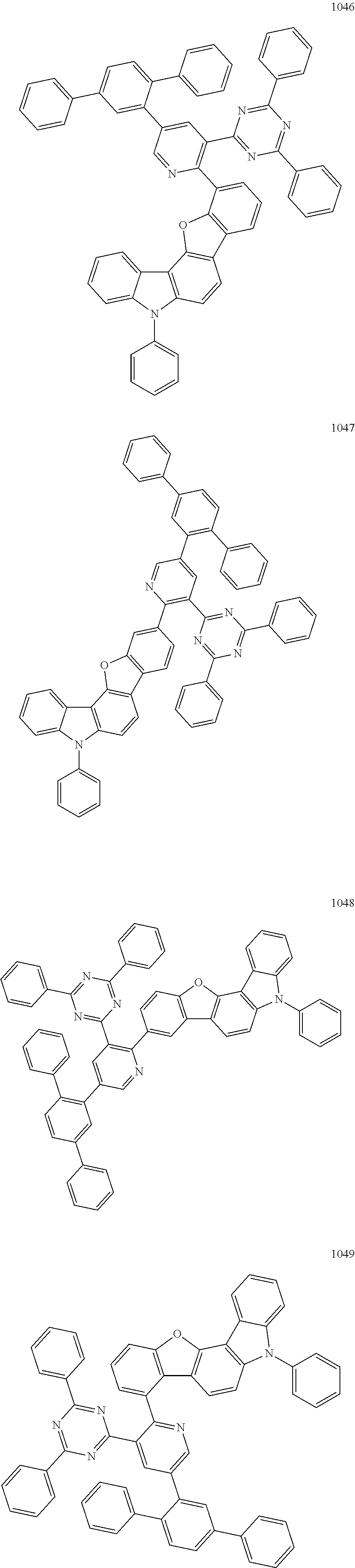 [SMILES: c1ccc(-c2ccc(-c3ccccc3)c(-c3cnc(-c4cccc5c4oc4c5ccc5c4c4ccccc4n5-c4ccccc4)c(-c4nc(-c5ccccc5)nc(-c5ccccc5)n4)c3)c2)cc1 ; c1ccc(-c2ccc(-c3ccccc3)c(-c3cnc(-c4ccc5c(c4)oc4c5ccc5c4c4ccccc4n5-c4ccccc4)c(-c4nc(-c5ccccc5)nc(-c5ccccc5)n4)c3)c2)cc1 ; c1ccc(-c2ccc(-c3ccccc3)c(-c3cnc(-c4ccc5oc6c(ccc7c6c6ccccc6n7-c6ccccc6)c5c4)c(-c4nc(-c5ccccc5)nc(-c5ccccc5)n4)c3)c2)cc1 ; c1ccc(-c2ccc(-c3ccccc3)c(-c3cnc(-c4cccc5oc6c(ccc7c6c6ccccc6n7-c6ccccc6)c45)c(-c4nc(-c5ccccc5)nc(-c5ccccc5)n4)c3)c2)cc1]

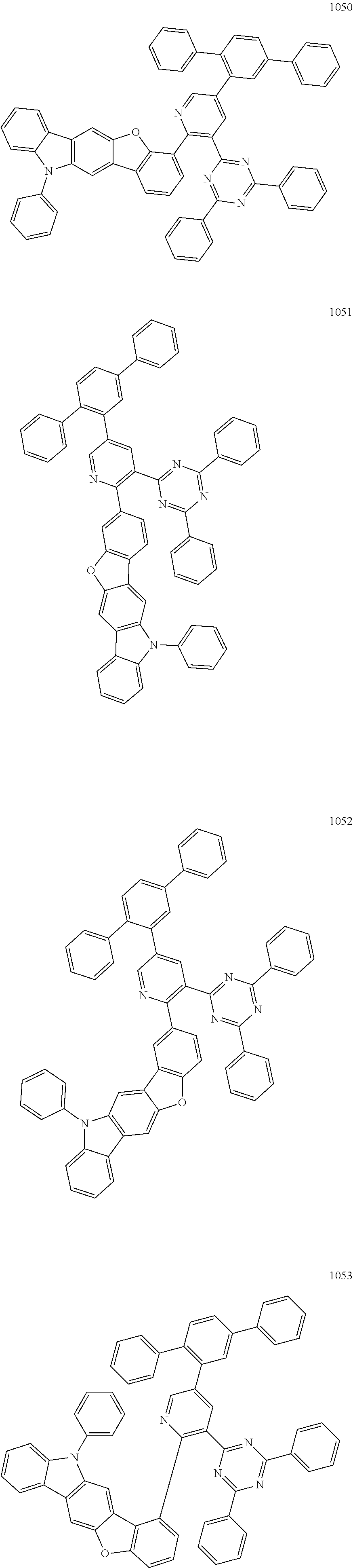 [SMILES: c1ccc(-c2ccc(-c3ccccc3)c(-c3cnc(-c4cccc5c4oc4cc6c7ccccc7n(-c7ccccc7)c6cc45)c(-c4nc(-c5ccccc5)nc(-c5ccccc5)n4)c3)c2)cc1 ; c1ccc(-c2ccc(-c3ccccc3)c(-c3cnc(-c4ccc5c(c4)oc4cc6c7ccccc7n(-c7ccccc7)c6cc45)c(-c4nc(-c5ccccc5)nc(-c5ccccc5)n4)c3)c2)cc1 ; c1ccc(-c2ccc(-c3ccccc3)c(-c3cnc(-c4ccc5oc6cc7c8ccccc8n(-c8ccccc8)c7cc6c5c4)c(-c4nc(-c5ccccc5)nc(-c5ccccc5)n4)c3)c2)cc1 ; c1ccc(-c2ccc(-c3ccccc3)c(-c3cnc(-c4cccc5oc6cc7c8ccccc8n(-c8ccccc8)c7cc6c45)c(-c4nc(-c5ccccc5)nc(-c5ccccc5)n4)c3)c2)cc1]

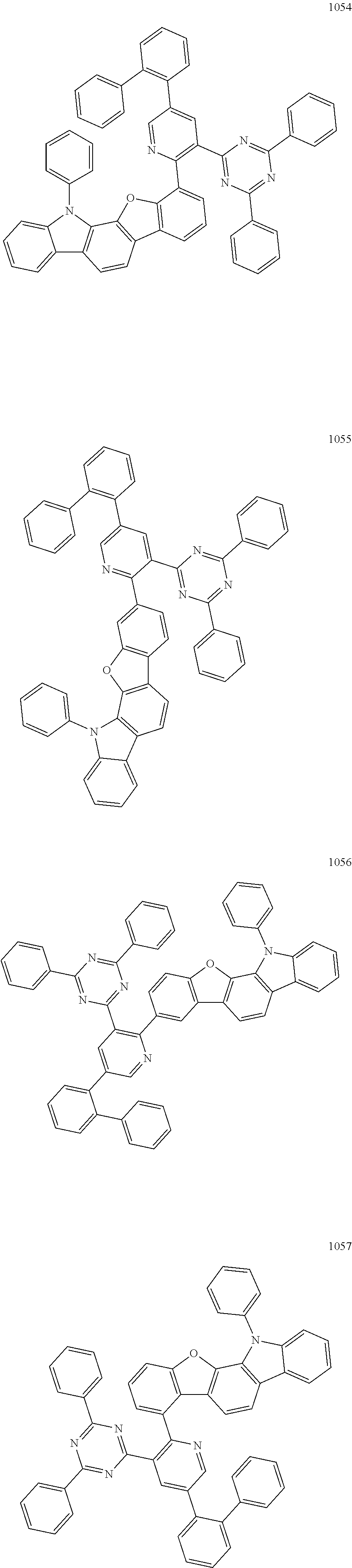 [SMILES: c1ccc(-c2nc(-c3ccccc3)nc(-c3cc(-c4ccccc4-c4ccccc4)cnc3-c3cccc4c3oc3c4ccc4c5ccccc5n(-c5ccccc5)c43)n2)cc1 ; c1ccc(-c2nc(-c3ccccc3)nc(-c3cc(-c4ccccc4-c4ccccc4)cnc3-c3ccc4c(c3)oc3c4ccc4c5ccccc5n(-c5ccccc5)c43)n2)cc1 ; c1ccc(-c2nc(-c3ccccc3)nc(-c3cc(-c4ccccc4-c4ccccc4)cnc3-c3ccc4oc5c(ccc6c7ccccc7n(-c7ccccc7)c65)c4c3)n2)cc1 ; c1ccc(-c2nc(-c3ccccc3)nc(-c3cc(-c4ccccc4-c4ccccc4)cnc3-c3cccc4oc5c(ccc6c7ccccc7n(-c7ccccc7)c65)c34)n2)cc1]

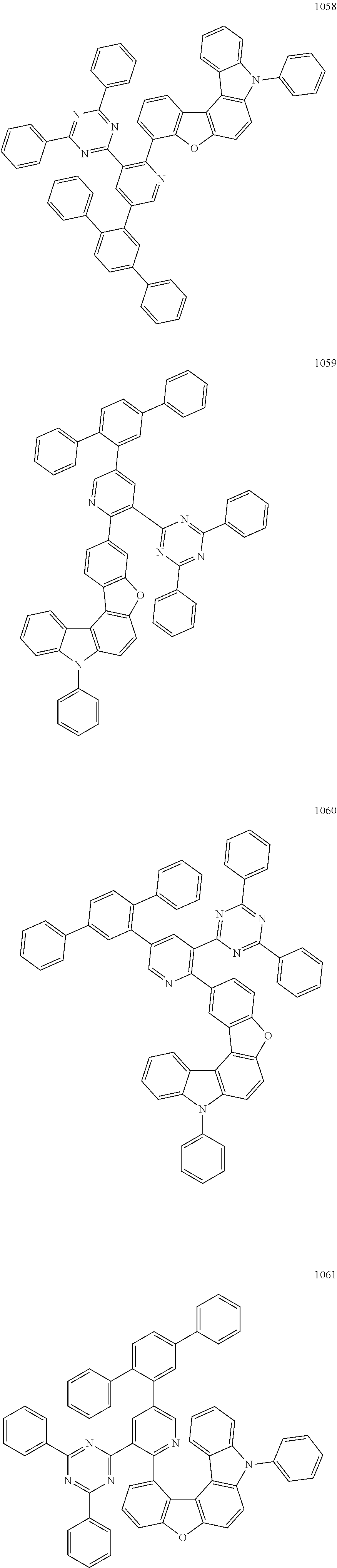 [SMILES: c1ccc(-c2ccc(-c3ccccc3)c(-c3cnc(-c4cccc5c4oc4ccc6c(c7ccccc7n6-c6ccccc6)c45)c(-c4nc(-c5ccccc5)nc(-c5ccccc5)n4)c3)c2)cc1 ; c1ccc(-c2ccc(-c3ccccc3)c(-c3cnc(-c4ccc5c(c4)oc4ccc6c(c7ccccc7n6-c6ccccc6)c45)c(-c4nc(-c5ccccc5)nc(-c5ccccc5)n4)c3)c2)cc1 ; c1ccc(-c2ccc(-c3ccccc3)c(-c3cnc(-c4ccc5oc6ccc7c(c8ccccc8n7-c7ccccc7)c6c5c4)c(-c4nc(-c5ccccc5)nc(-c5ccccc5)n4)c3)c2)cc1 ; c1ccc(-c2ccc(-c3ccccc3)c(-c3cnc(-c4cccc5oc6ccc7c(c8ccccc8n7-c7ccccc7)c6c45)c(-c4nc(-c5ccccc5)nc(-c5ccccc5)n4)c3)c2)cc1]

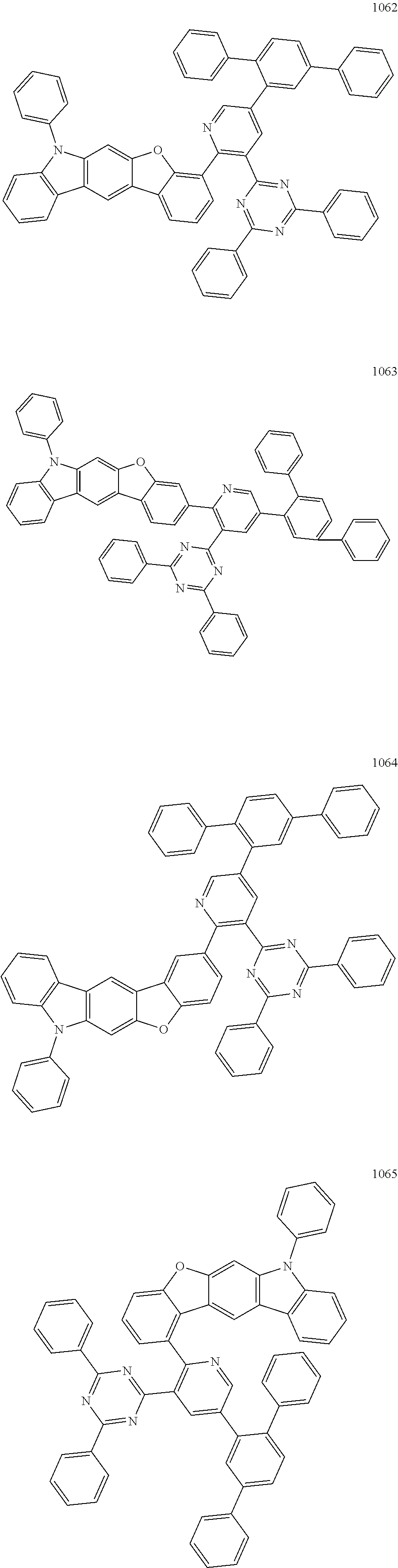 [SMILES: c1ccc(-c2ccc(-c3ccccc3)c(-c3cnc(-c4cccc5c4oc4cc6c(cc45)c4ccccc4n6-c4ccccc4)c(-c4nc(-c5ccccc5)nc(-c5ccccc5)n4)c3)c2)cc1 ; c1ccc(-c2ccc(-c3ccccc3)c(-c3cnc(-c4ccc5c(c4)oc4cc6c(cc45)c4ccccc4n6-c4ccccc4)c(-c4nc(-c5ccccc5)nc(-c5ccccc5)n4)c3)c2)cc1 ; c1ccc(-c2ccc(-c3ccccc3)c(-c3cnc(-c4ccc5oc6cc7c(cc6c5c4)c4ccccc4n7-c4ccccc4)c(-c4nc(-c5ccccc5)nc(-c5ccccc5)n4)c3)c2)cc1 ; c1ccc(-c2ccc(-c3ccccc3)c(-c3cnc(-c4cccc5oc6cc7c(cc6c45)c4ccccc4n7-c4ccccc4)c(-c4nc(-c5ccccc5)nc(-c5ccccc5)n4)c3)c2)cc1]

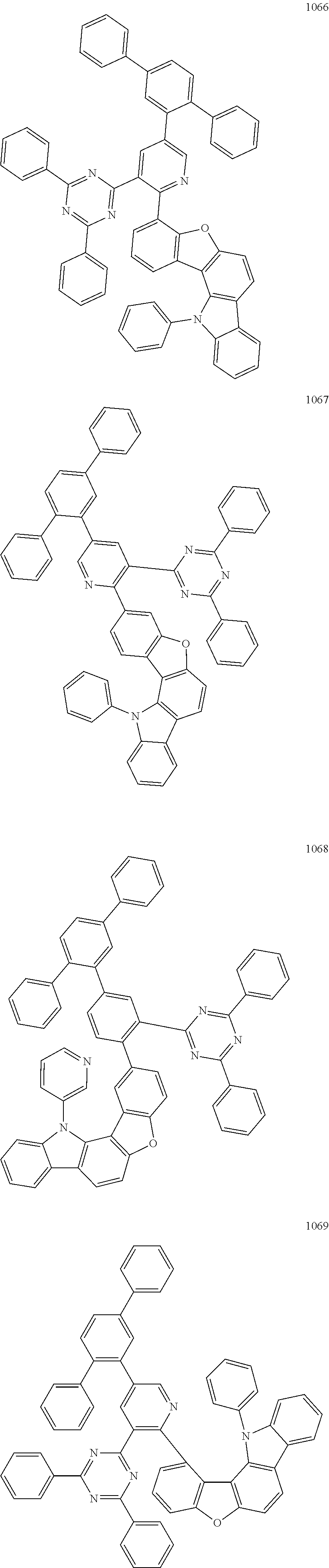 [SMILES: c1ccc(-c2ccc(-c3ccccc3)c(-c3cnc(-c4cccc5c4oc4ccc6c7ccccc7n(-c7ccccc7)c6c45)c(-c4nc(-c5ccccc5)nc(-c5ccccc5)n4)c3)c2)cc1 ; c1ccc(-c2ccc(-c3ccccc3)c(-c3cnc(-c4ccc5c(c4)oc4ccc6c7ccccc7n(-c7ccccc7)c6c45)c(-c4nc(-c5ccccc5)nc(-c5ccccc5)n4)c3)c2)cc1 ; c1ccc(-c2ccc(-c3ccccc3)c(-c3ccc(-c4ccc5oc6ccc7c8ccccc8n(-c8cccnc8)c7c6c5c4)c(-c4nc(-c5ccccc5)nc(-c5ccccc5)n4)c3)c2)cc1 ; c1ccc(-c2ccc(-c3ccccc3)c(-c3cnc(-c4cccc5oc6ccc7c8ccccc8n(-c8ccccc8)c7c6c45)c(-c4nc(-c5ccccc5)nc(-c5ccccc5)n4)c3)c2)cc1]

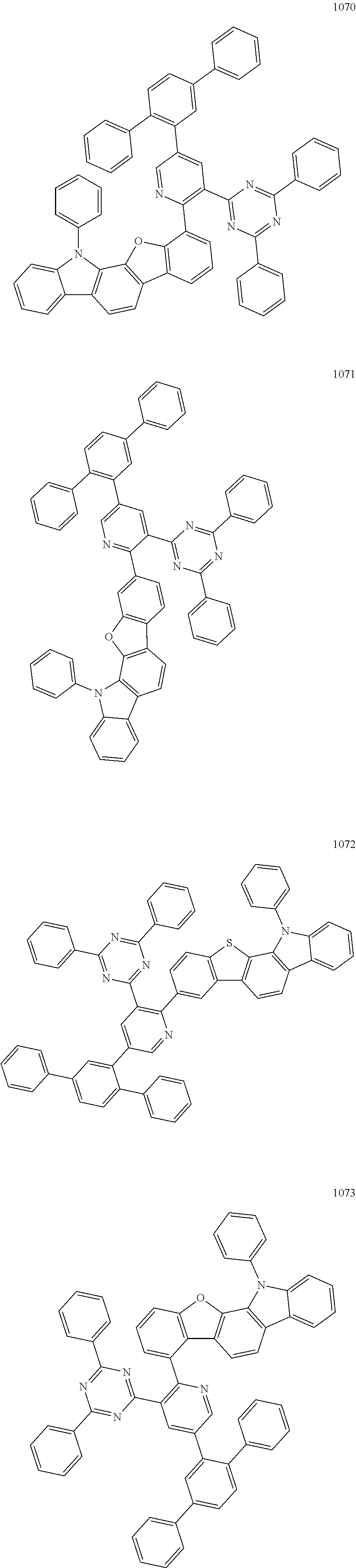 [SMILES: c1ccc(-c2ccc(-c3ccccc3)c(-c3cnc(-c4cccc5c4oc4c5ccc5c6ccccc6n(-c6ccccc6)c54)c(-c4nc(-c5ccccc5)nc(-c5ccccc5)n4)c3)c2)cc1 ; c1ccc(-c2ccc(-c3ccccc3)c(-c3cnc(-c4ccc5c(c4)oc4c5ccc5c6ccccc6n(-c6ccccc6)c54)c(-c4nc(-c5ccccc5)nc(-c5ccccc5)n4)c3)c2)cc1 ; c1ccc(-c2ccc(-c3ccccc3)c(-c3cnc(-c4ccc5sc6c(ccc7c8ccccc8n(-c8ccccc8)c76)c5c4)c(-c4nc(-c5ccccc5)nc(-c5ccccc5)n4)c3)c2)cc1 ; c1ccc(-c2ccc(-c3ccccc3)c(-c3cnc(-c4cccc5oc6c(ccc7c8ccccc8n(-c8ccccc8)c76)c45)c(-c4nc(-c5ccccc5)nc(-c5ccccc5)n4)c3)c2)cc1]

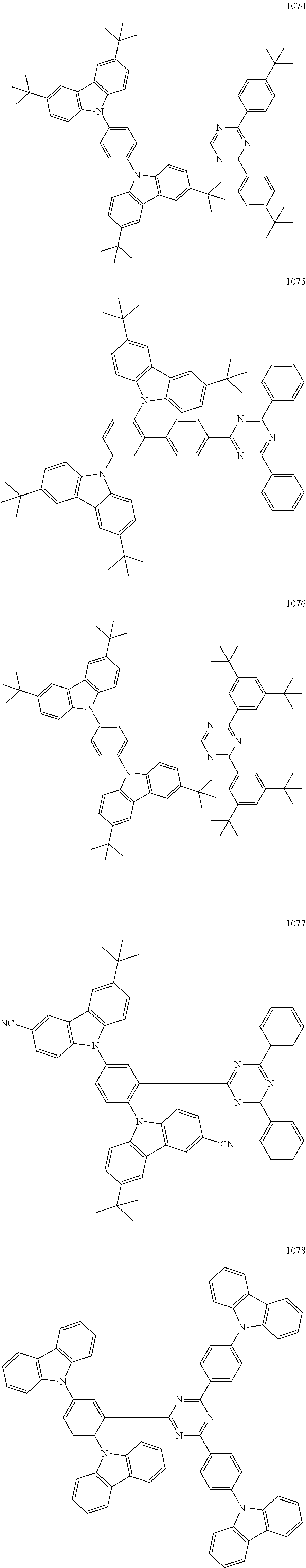

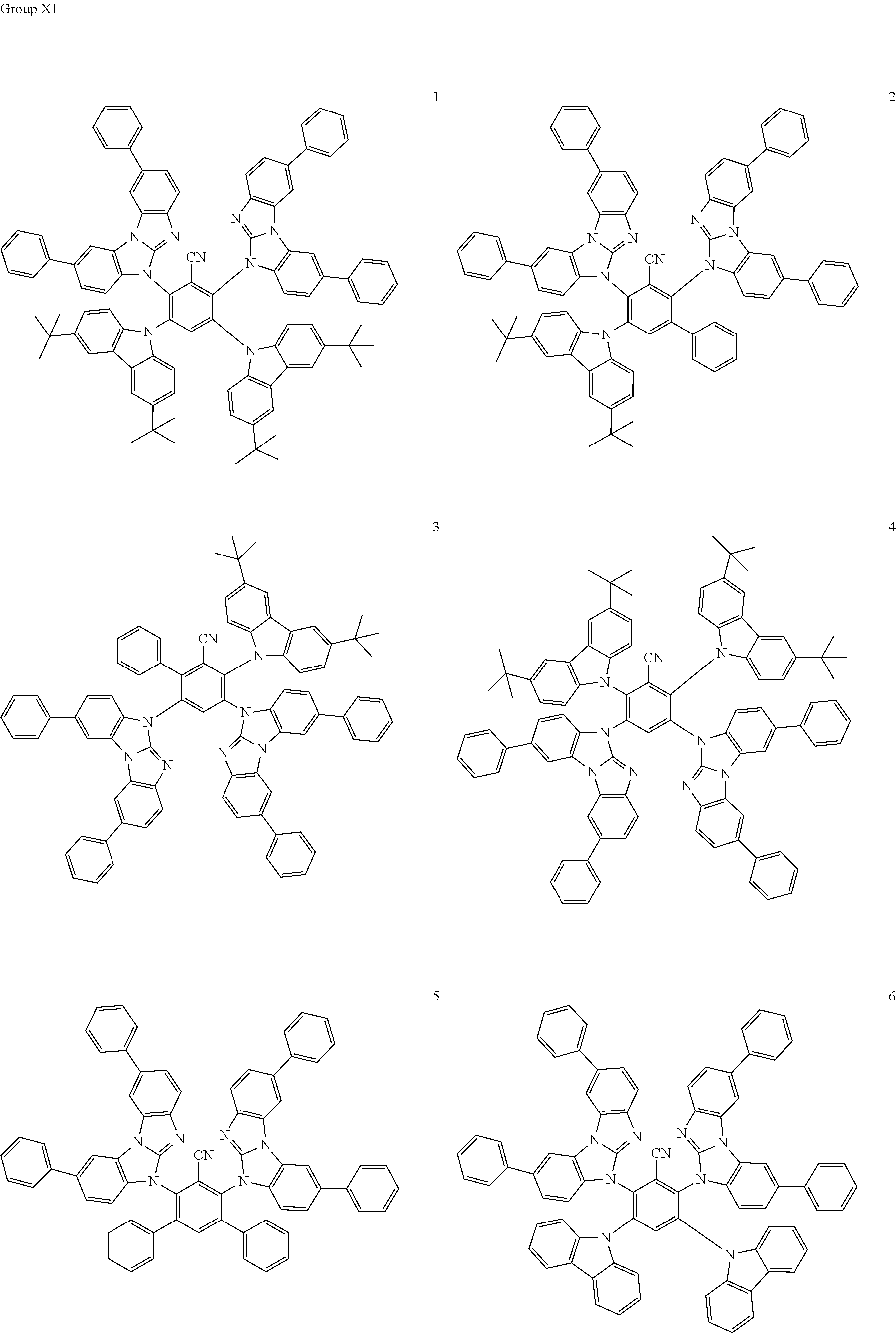

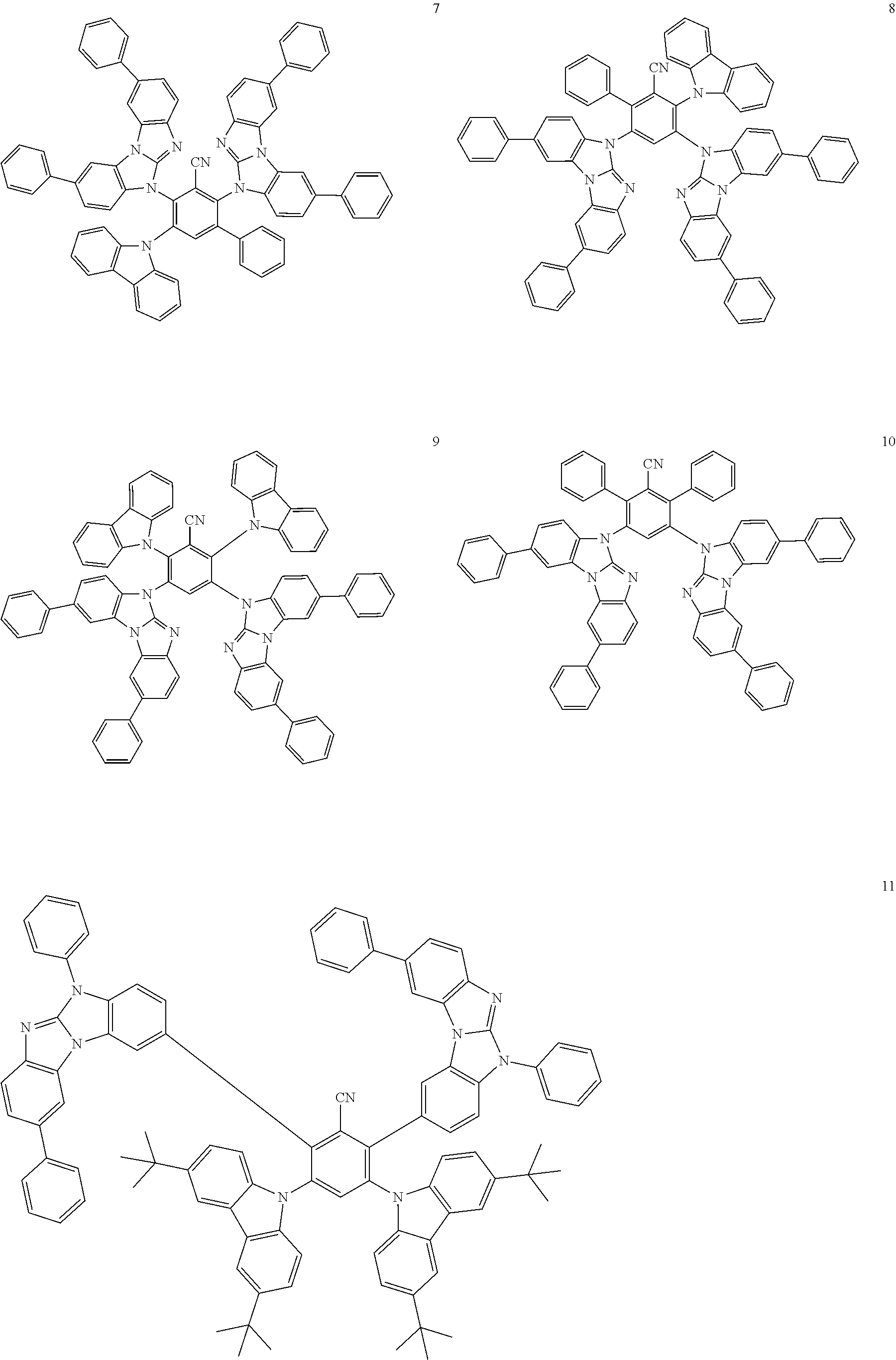

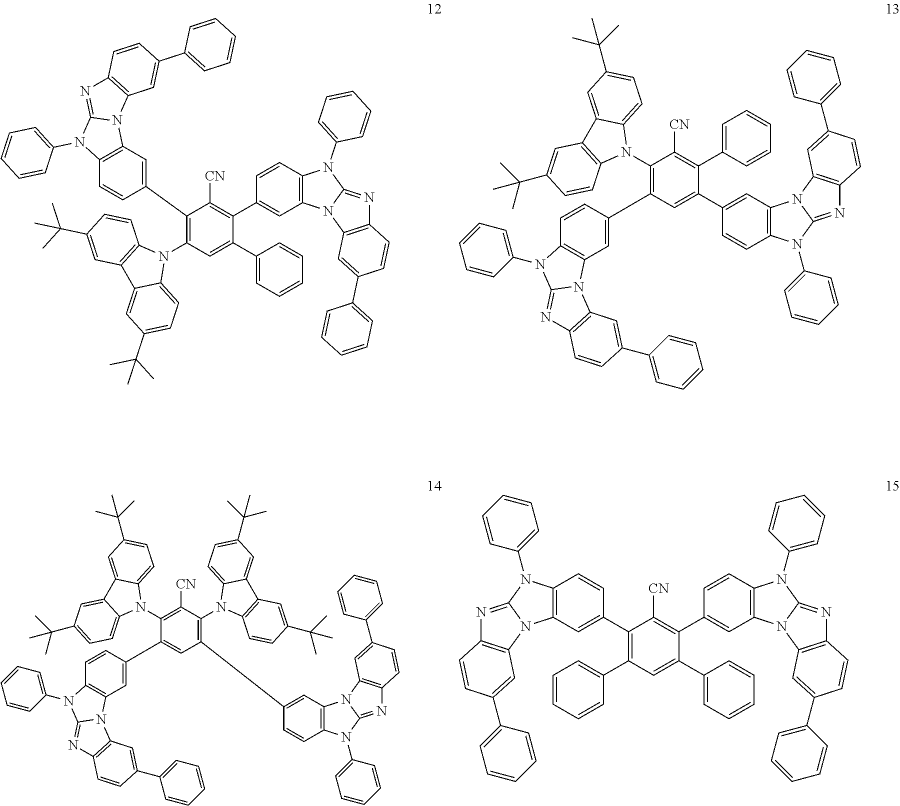

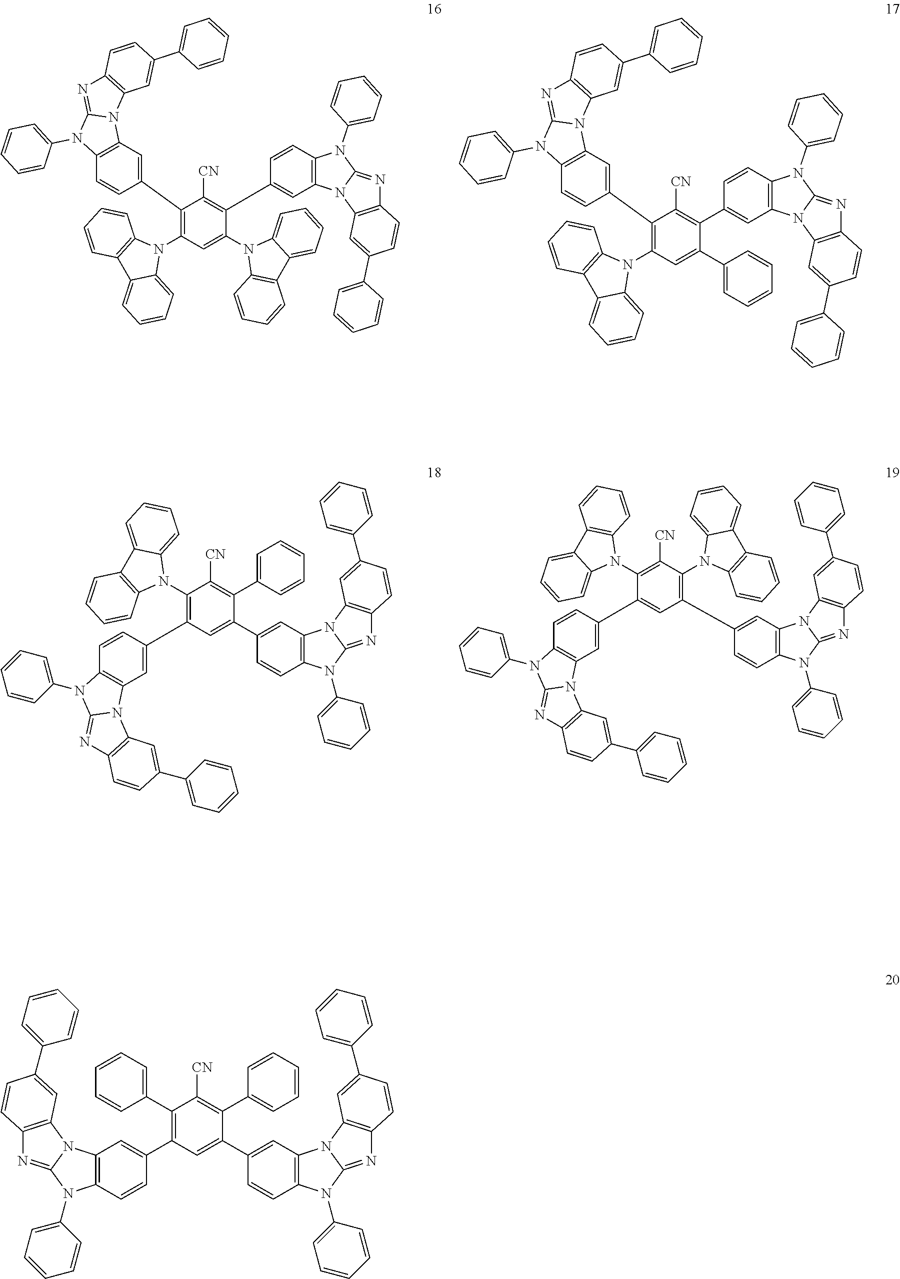

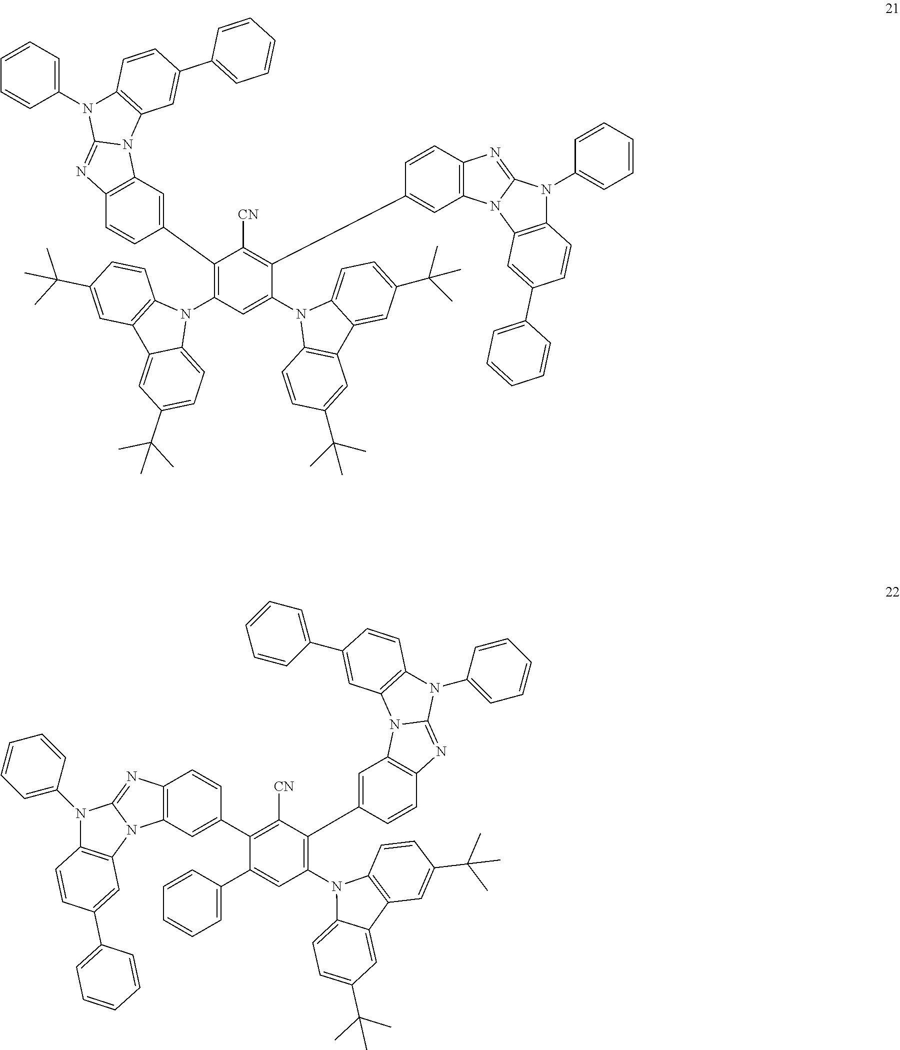 [SMILES: CC(C)(C)c1ccc2c(c1)c1cc(C(C)(C)C)ccc1n2-c1cc(-n2c3ccc(C(C)(C)C)cc3c3cc(C(C)(C)C)ccc32)c(-c2ccc3nc4n(-c5ccccc5)c5ccc(-c6ccccc6)cc5n4c3c2)c(C#N)c1-c1ccc2nc3n(-c4ccccc4)c4ccc(-c5ccccc5)cc4n3c2c1 ; CC(C)(C)c1ccc2c(c1)c1cc(C(C)(C)C)ccc1n2-c1cc(-c2ccccc2)c(-c2ccc3nc4n(-c5ccccc5)c5ccc(-c6ccccc6)cc5n4c3c2)c(C#N)c1-c1ccc2nc3n(-c4ccccc4)c4ccc(-c5ccccc5)cc4n3c2c1]

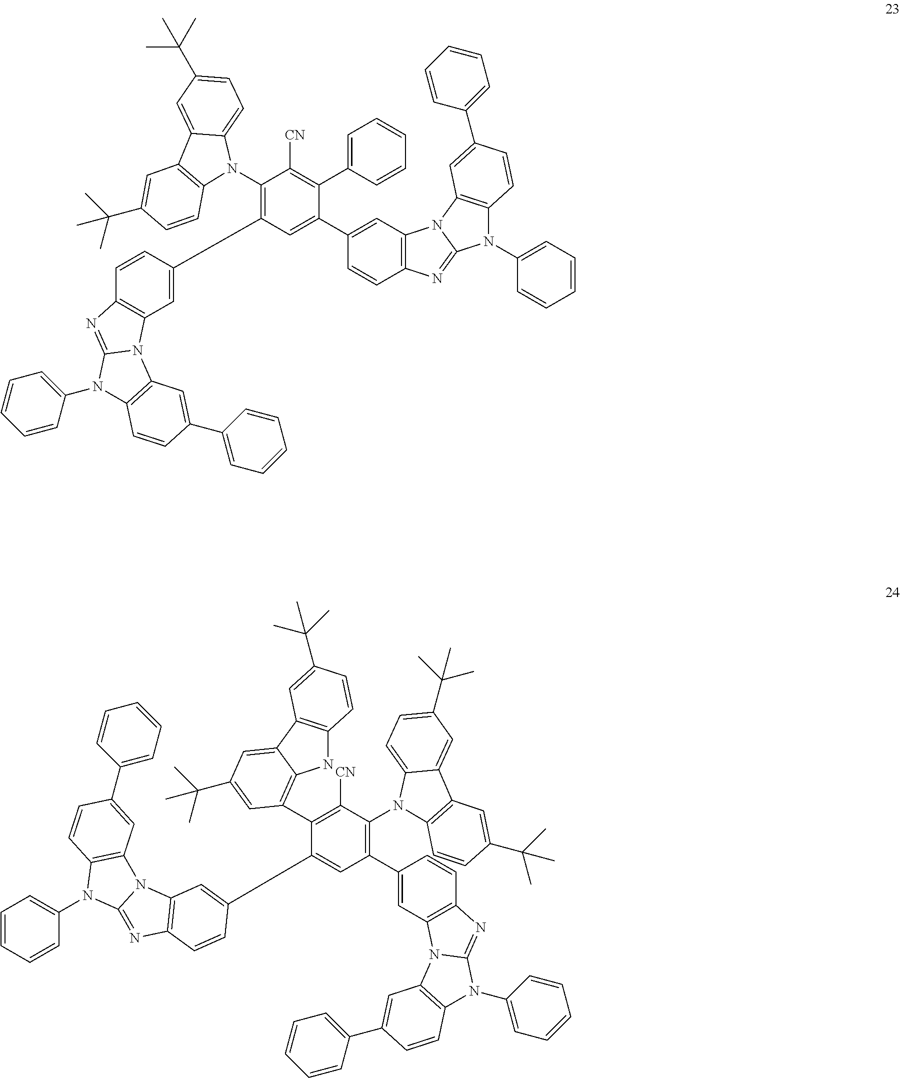 [SMILES: CC(C)(C)c1ccc2c(c1)c1cc(C(C)(C)C)ccc1n2-c1c(-c2ccc3nc4n(-c5ccccc5)c5ccc(-c6ccccc6)cc5n4c3c2)cc(-c2ccc3nc4n(-c5ccccc5)c5ccc(-c6ccccc6)cc5n4c3c2)c(-c2ccccc2)c1C#N ; CC(C)(C)c1ccc2[nH]c3c(-c4c(-c5ccc6nc7n(-c8ccccc8)c8ccc(-c9ccccc9)cc8n7c6c5)cc(-c5ccc6nc7n(-c8ccccc8)c8ccc(-c9ccccc9)cc8n7c6c5)c(-n5c6ccc(C(C)(C)C)cc6c6cc(C(C)(C)C)ccc65)c4C#N)cc(C(C)(C)C)cc3c2c1]

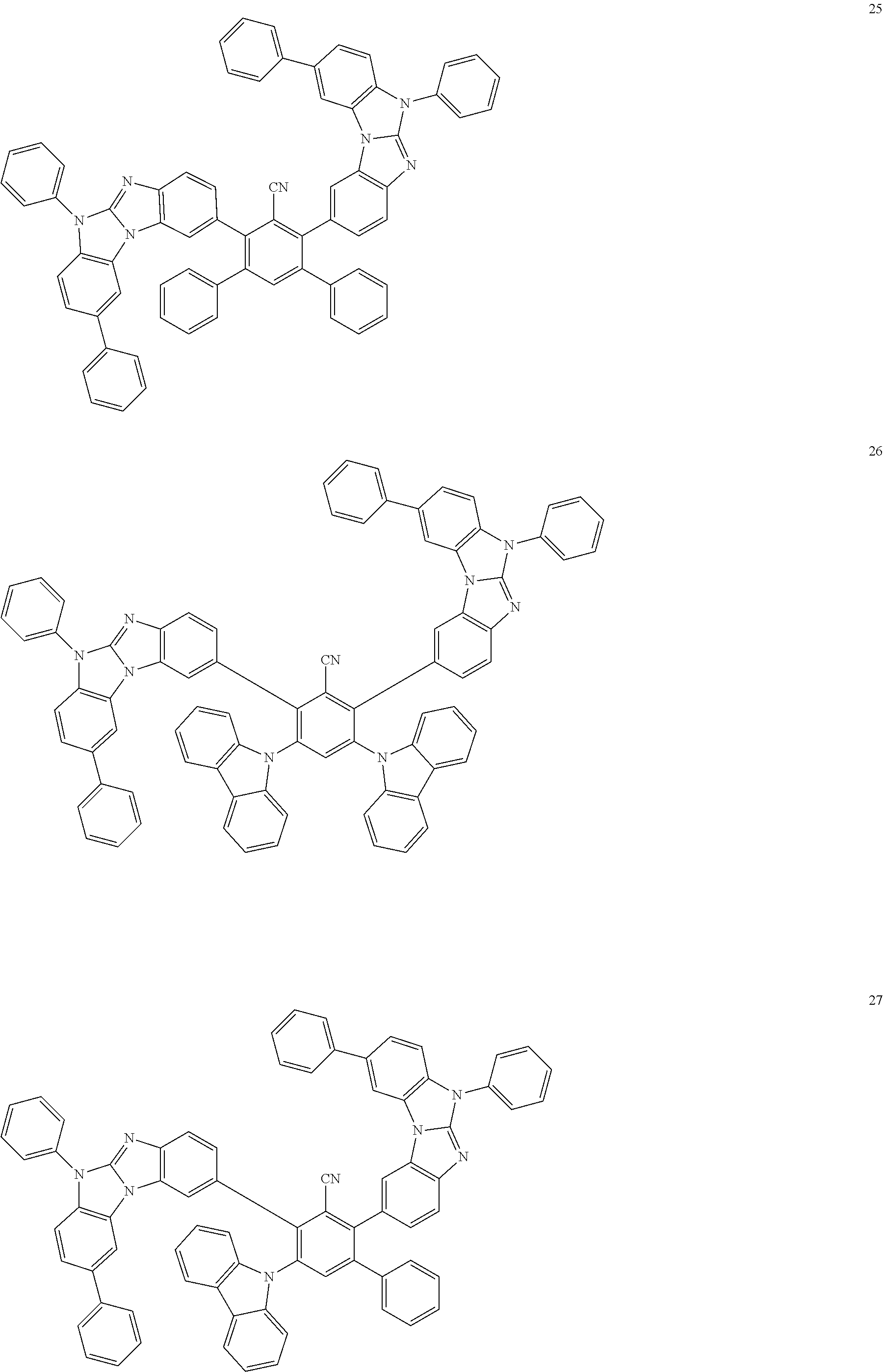 [SMILES: N#Cc1c(-c2ccc3nc4n(-c5ccccc5)c5ccc(-c6ccccc6)cc5n4c3c2)c(-c2ccccc2)cc(-c2ccccc2)c1-c1ccc2nc3n(-c4ccccc4)c4ccc(-c5ccccc5)cc4n3c2c1 ; N#Cc1c(-c2ccc3nc4n(-c5ccccc5)c5ccc(-c6ccccc6)cc5n4c3c2)c(-n2c3ccccc3c3ccccc32)cc(-n2c3ccccc3c3ccccc32)c1-c1ccc2nc3n(-c4ccccc4)c4ccc(-c5ccccc5)cc4n3c2c1 ; N#Cc1c(-c2ccc3nc4n(-c5ccccc5)c5ccc(-c6ccccc6)cc5n4c3c2)c(-c2ccccc2)cc(-n2c3ccccc3c3ccccc32)c1-c1ccc2nc3n(-c4ccccc4)c4ccc(-c5ccccc5)cc4n3c2c1]

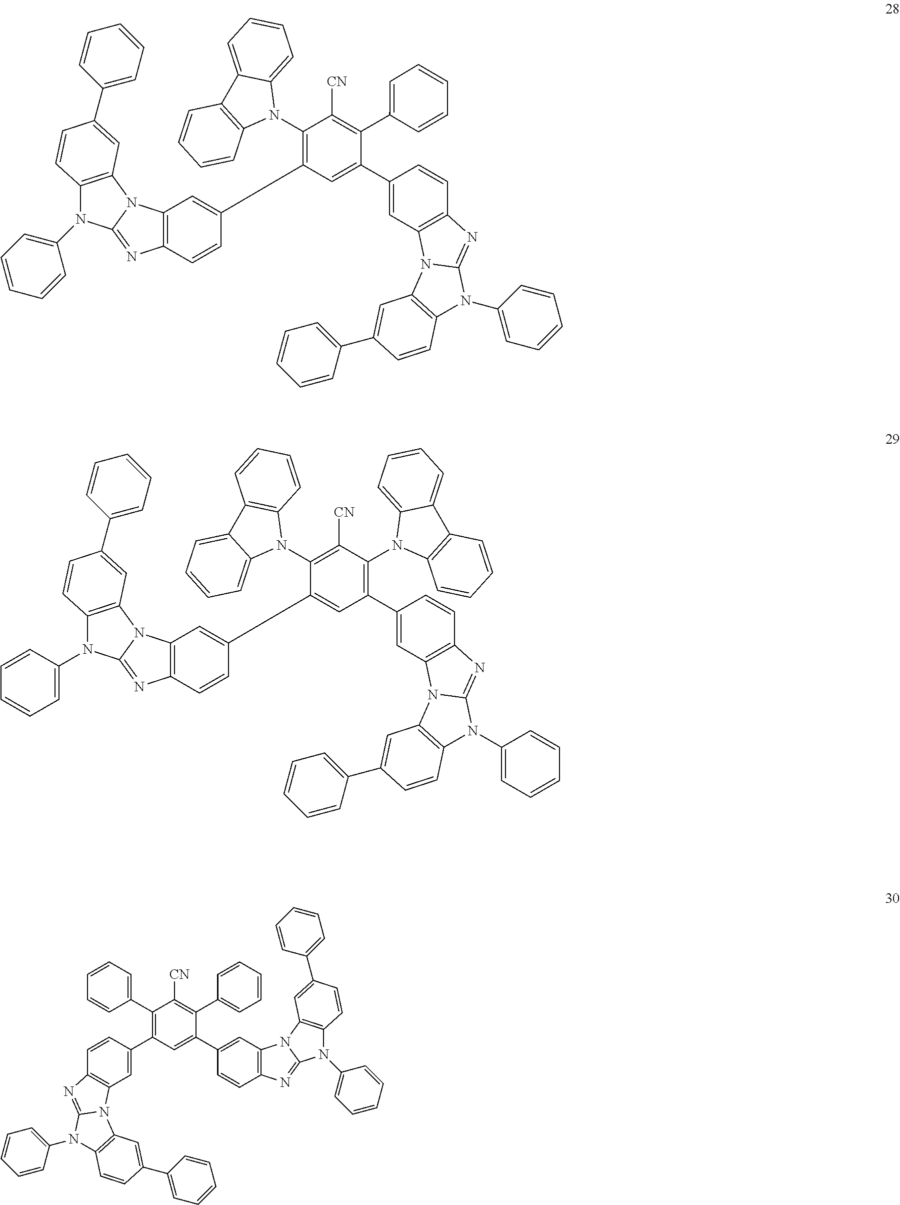 [SMILES: N#Cc1c(-c2ccccc2)c(-c2ccc3nc4n(-c5ccccc5)c5ccc(-c6ccccc6)cc5n4c3c2)cc(-c2ccc3nc4n(-c5ccccc5)c5ccc(-c6ccccc6)cc5n4c3c2)c1-n1c2ccccc2c2ccccc21 ; N#Cc1c(-n2c3ccccc3c3ccccc32)c(-c2ccc3nc4n(-c5ccccc5)c5ccc(-c6ccccc6)cc5n4c3c2)cc(-c2ccc3nc4n(-c5ccccc5)c5ccc(-c6ccccc6)cc5n4c3c2)c1-n1c2ccccc2c2ccccc21 ; N#Cc1c(-c2ccccc2)c(-c2ccc3nc4n(-c5ccccc5)c5ccc(-c6ccccc6)cc5n4c3c2)cc(-c2ccc3nc4n(-c5ccccc5)c5ccc(-c6ccccc6)cc5n4c3c2)c1-c1ccccc1]

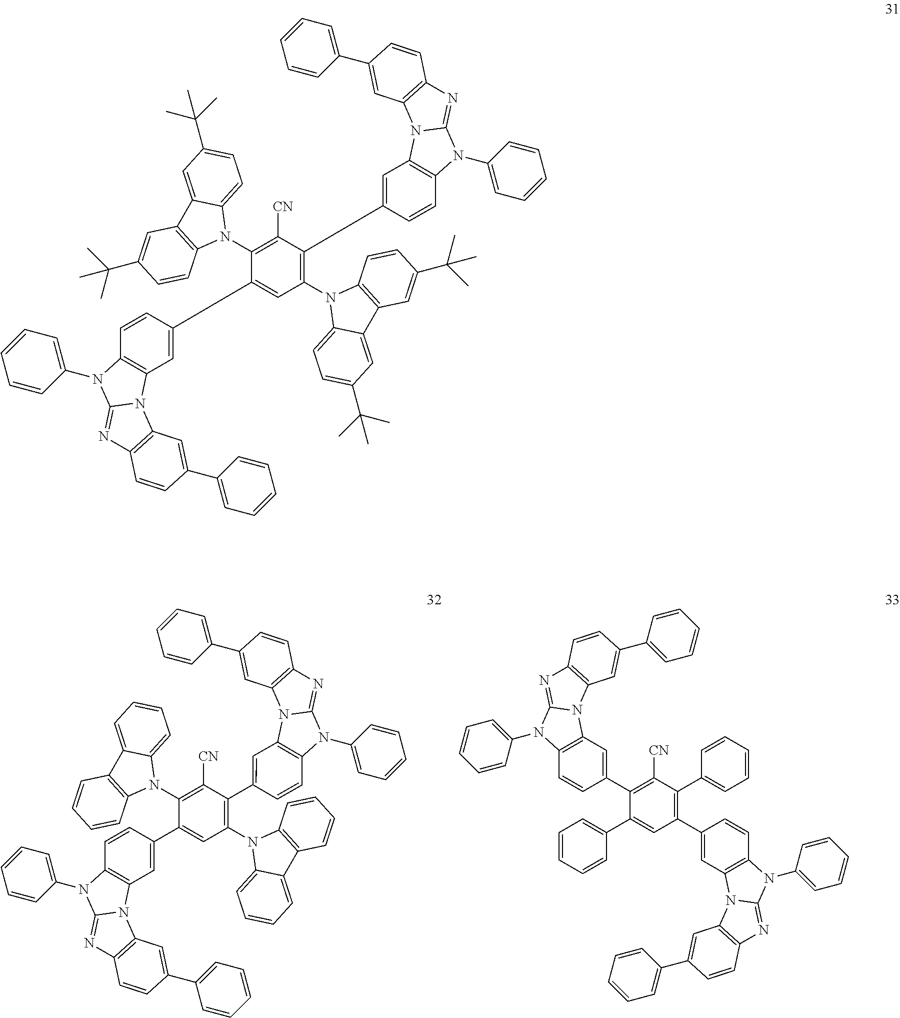 [SMILES: CC(C)(C)c1ccc2c(c1)c1cc(C(C)(C)C)ccc1n2-c1cc(-c2ccc3c(c2)n2c4cc(-c5ccccc5)ccc4nc2n3-c2ccccc2)c(-n2c3ccc(C(C)(C)C)cc3c3cc(C(C)(C)C)ccc32)c(C#N)c1-c1ccc2c(c1)n1c3cc(-c4ccccc4)ccc3nc1n2-c1ccccc1 ; N#Cc1c(-c2ccc3c(c2)n2c4cc(-c5ccccc5)ccc4nc2n3-c2ccccc2)c(-n2c3ccccc3c3ccccc32)cc(-c2ccc3c(c2)n2c4cc(-c5ccccc5)ccc4nc2n3-c2ccccc2)c1-n1c2ccccc2c2ccccc21 ; N#Cc1c(-c2ccccc2)c(-c2ccc3c(c2)n2c4cc(-c5ccccc5)ccc4nc2n3-c2ccccc2)cc(-c2ccccc2)c1-c1ccc2c(c1)n1c3cc(-c4ccccc4)ccc3nc1n2-c1ccccc1]

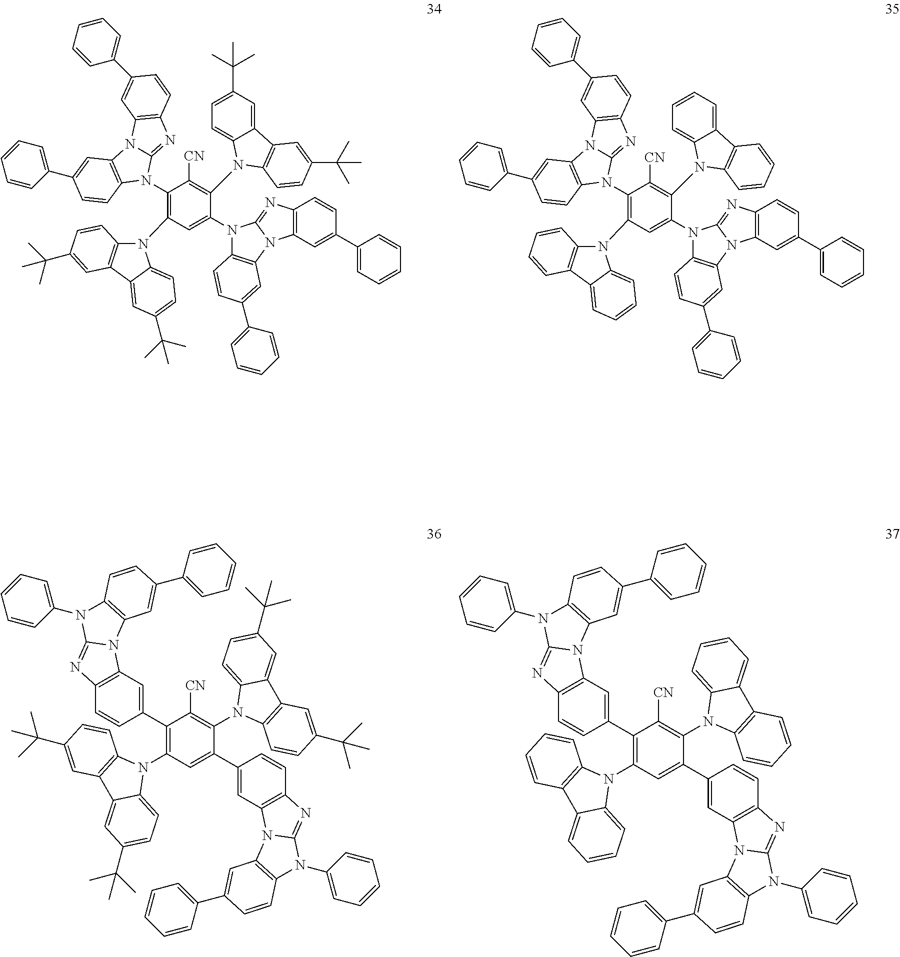

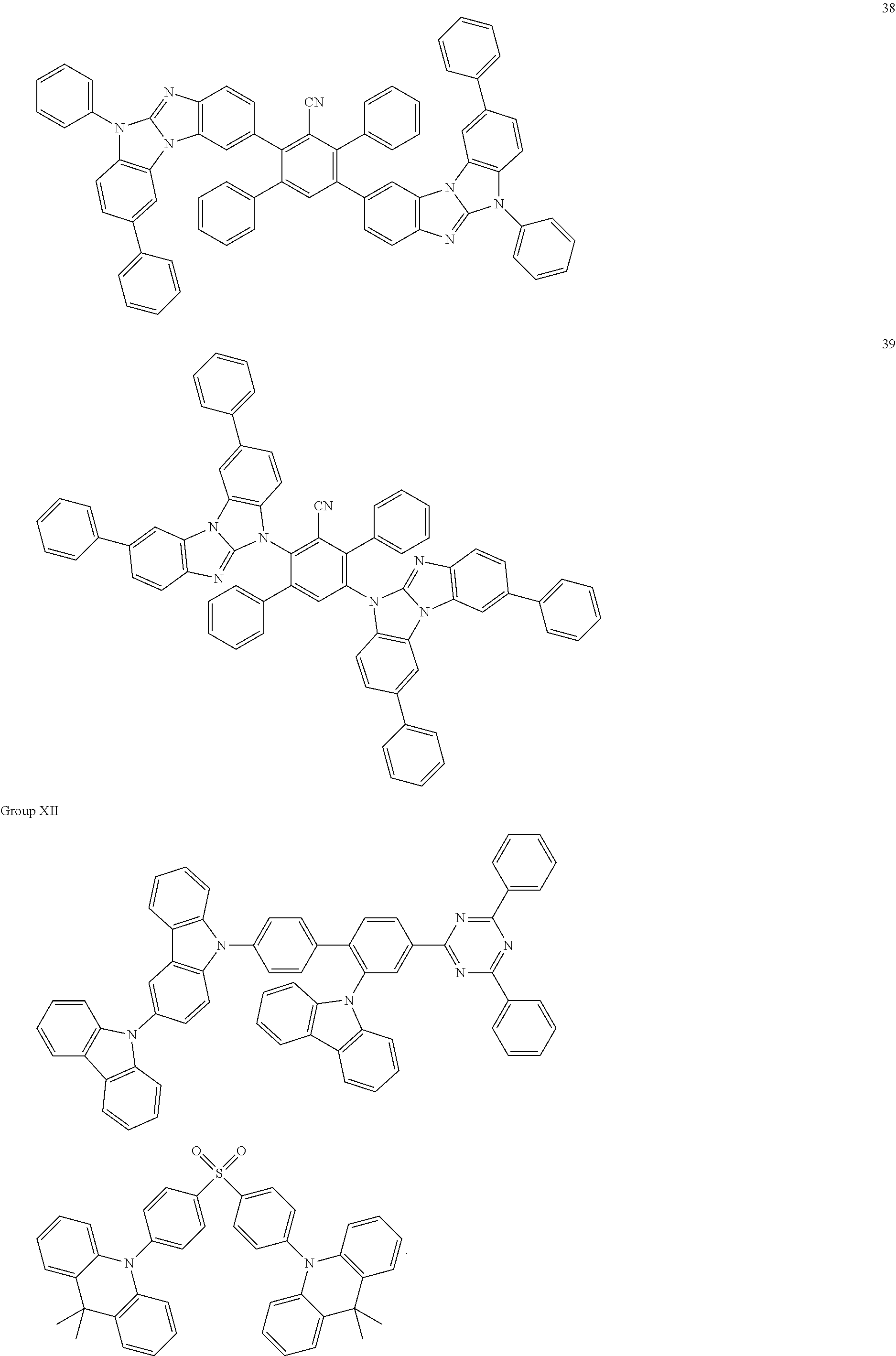 [SMILES: N#Cc1c(-c2ccccc2)c(-c2ccc3nc4n(-c5ccccc5)c5ccc(-c6ccccc6)cc5n4c3c2)cc(-c2ccccc2)c1-c1ccc2nc3n(-c4ccccc4)c4ccc(-c5ccccc5)cc4n3c2c1 ; N#Cc1c(-c2ccccc2)c(-n2c3ccc(-c4ccccc4)cc3n3c4cc(-c5ccccc5)ccc4nc23)cc(-c2ccccc2)c1-n1c2ccc(-c3ccccc3)cc2n2c3cc(-c4ccccc4)ccc3nc12 ; c1ccc(-c2nc(-c3ccccc3)nc(-c3ccc(-c4ccc(-n5c6ccccc6c6cc(-n7c8ccccc8c8ccccc87)ccc65)cc4)c(-n4c5ccccc5c5ccccc54)c3)n2)cc1 ; CC1(C)c2ccccc2N(c2ccc(S(=O)(=O)c3ccc(N4c5ccccc5C(C)(C)c5ccccc54)cc3)cc2)c2ccccc21]

Hole transport region 12

In the organic light-emitting device 10, the hole transport region 12 may be between the first electrode 11 and the emission layer 15.

The hole transport region 12 may have a single-layered structure or a multi-layered structure.

For example, the hole transport region 12 may have a structure of hole injection layer, a structure of hole transport layer, a structure of hole injection layer/hole transport layer, a structure of hole injection layer/first hole transport layer/second hole transport layer, a structure of hole transport layer/intermediate layer, a structure of hole injection layer/hole transport layer/intermediate layer, a structure of hole transport layer/electron blocking layer, or a structure of hole injection layer/hole transport layer/electron blocking layer, but embodiments are not limited thereto.

The hole transport region 12 may include a compound having hole transport characteristics.

For example, the hole transport region 12 may include an amine-based compound.

In an embodiment, the hole transport region 12 may include at least one compound represented by Formulae 201 to 205, but embodiments are not limited thereto:

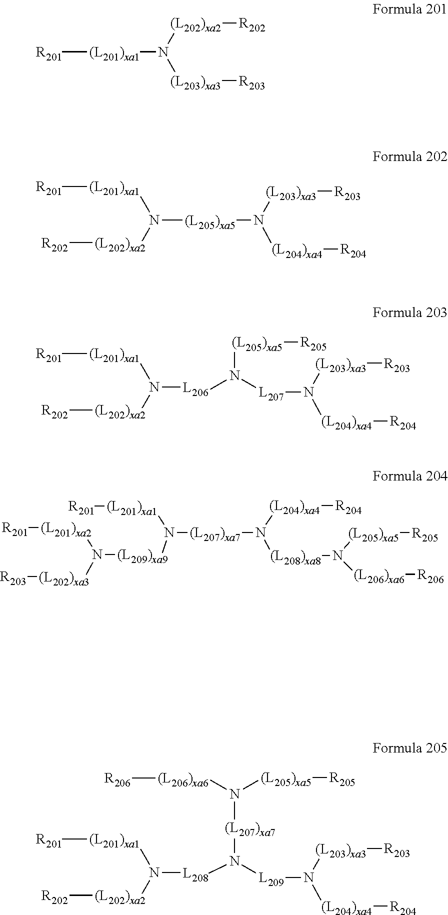

wherein, in Formulae 201 to 205,

L₂₀₁ to L₂₀₉ may each independently be *—O—*′, *—S—*′, a substituted or unsubstituted C₅-C₆₀ carbocyclic group or a substituted or unsubstituted C₁-C₆₀ heterocyclic group,

xa1 to xa9 may each independently be an integer from 0 to 5,

R₂₀₁ to R₂₀₆ may each independently be a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent aromatic condensed polycyclic group, a substituted or unsubstituted monovalent aromatic condensed heteropolycyclic group, a substituted or unsubstituted monovalent non-aromatic condensed polycyclic group, and a substituted or unsubstituted monovalent non-aromatic condensed heteropolycyclic group, and two adjacent groups R₂₀₁ to R₂₀₆ may optionally be bound to each other via a single bond, a dimethyl-methylene group, or a diphenyl-methylene group.

In some embodiments,

L₂₀₁ to L₂₀₉ may be a benzene group, a heptalene group, an indene group, a naphthalene group, an azulene group, a heptalene group, an indacene group, an acenaphthylene group, a fluorene group, a spiro-bifluorene group, a benzofluorene group, a dibenzofluorene group, a phenalene group, a phenanthrene group, an anthracene group, a fluoranthene group, a triphenylene group, a pyrene group, a chrysene group, a naphthacene group, a picene group, a perylene group, a pentacene group, a hexacene group, a pentacene group, a rubicene group, a coronene group, an ovalene group, a pyrrole group, an isoindole group, an indole group, a furan group, a thiophene group, a benzofuran group, a benzothiophene group, a benzocarbazole group, a dibenzocarbazole group, a dibenzofuran group, a dibenzothiophene group, a dibenzothiophene sulfone(dibenzothiophene sulfone) group, a carbazole group, a dibenzosilole group, an indenocarbazole group, an indolocarbazole group, a benzofurocarbazole group, a benzothienocarbazole group or a triindolobenzene group, each unsubstituted or substituted with at least one of deuterium, a C₁-C₁₀ alkyl group, a C₁-C₁₀ alkoxy group, a phenyl group, a naphthyl group, a fluorenyl group, a carbazolyl group, a dibenzofuranyl group, a dibenzothiophenyl group, a triphenylenyl group, a biphenyl group, a terphenyl group, a tetraphenyl group, —Si(Q₁₁)(Q₁₂)(Q₁₃), or any combination thereof,

xa1 to xa9 may each independently be 0, 1, or 2, and

R₂₀₁ to R₂₀₆ may each independently be a phenyl group, a biphenyl group, a terphenyl group, a pentalenyl group, an indenyl group, a naphthyl group, an azulenyl group, a heptalenyl group, an indacenyl group, an acenaphthyl group, a fluorenyl group, a spiro-bifluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a perylenyl group, a pentaphenyl group, a hexacenyl group, a pentacenyl group, a rubicenyl group, a coronenyl group, an ovalenyl group, a thiophenyl group, a furanyl group, a carbazolyl group, an indolyl group, an isoindolyl group, a benzofuranyl group, a benzothiophenyl group, a dibenzofuranyl group, a dibenzothiophenyl group, a benzocarbazolyl group, a dibenzocarbazolyl group, a dibenzosilolyl group, a pyridinyl group, an indenocarbazolyl group, an indolocarbazolyl group, a benzofurocarbazolyl group, or a benzothienocarbazolyl group, each unsubstituted or substituted with at least one of deuterium, —F, —Cl, —Br, —I, a hydroxyl group, a cyano group, a nitro group, an amidino group, a hydrazino group, a hydrazono group, a C₁-C₂₀ alkyl group, a C₁-C₂₀ alkoxy group, a cyclopentyl group, a cyclohexyl group, a cycloheptyl group, a cyclopentenyl group, a cyclohexenyl group, a phenyl group, a biphenyl group, a terphenyl group, a phenyl group substituted with a C₁-C₁₀ alkyl group, a phenyl group substituted with —F, a pentalenyl group, an indenyl group, a naphthyl group, an azulenyl group, a heptalenyl group, an indacenyl group, an acenaphthyl group, a fluorenyl group, a spiro-bifluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a perylenyl group, a pentaphenyl group, a hexacenyl group, a pentacenyl group, a rubicenyl group, a coronenyl group, an ovalenyl group, a thiophenyl group, a furanyl group, a carbazolyl group, an indolyl group, an isoindolyl group, a benzofuranyl group, a benzothiophenyl group, a dibenzofuranyl group, a dibenzothiophenyl group, a benzocarbazolyl group, a dibenzocarbazolyl group, a dibenzosilolyl group, a pyridinyl group, —Si(Q₃₁)(Q₃₂)(Q₃₃), —N(Q₃₁)(Q₃₂), or any combination thereof,

wherein Q₁₁ to Q₁₃ and Q₃₁ to Q₃₃ may each independently be a C₁-C₁₀ alkyl group, a C₁-C₁₀ alkoxy group, a phenyl group, a biphenyl group, a terphenyl group, or a naphthyl group.

According to an embodiment, the hole transport region 12 may include a carbazole-containing amine-based compound.

In one or more embodiments, the hole transport region 12 may include a carbazole-containing amine-based compound and a carbazole-free amine-based compound.

The carbazole-containing amine-based compound may be, for example, a compound represented by Formula 201 including a carbazole group and further including at least one of a dibenzofuran group, a dibenzothiophene group, a fluorene group, a spirofluorene group, an indenocarbazole group, an indolocarbazole group, a benzofurocarbazole group, a benzothienocarbazole group, or any combination thereof.

The carbazole-free amine-based compound may be, for example, compounds represented by Formula 201 not including a carbazole group and including at least one of a dibenzofuran group, a dibenzothiophene group, a fluorene group, a spirofluorene group, an indenocarbazole group, an indolocarbazole group, a benzofurocarbazole group, a benzothienocarbazole group, or any combination thereof.

In one or more embodiments, the hole transport region 12 may include at least one compound represented by Formula 201, 202, or a combination thereof.

In an embodiment, the hole transport region 12 may include at least one compound represented by Formulae 201-1, 202-1, 201-2, or any combination thereof, but embodiments are not limited thereto:

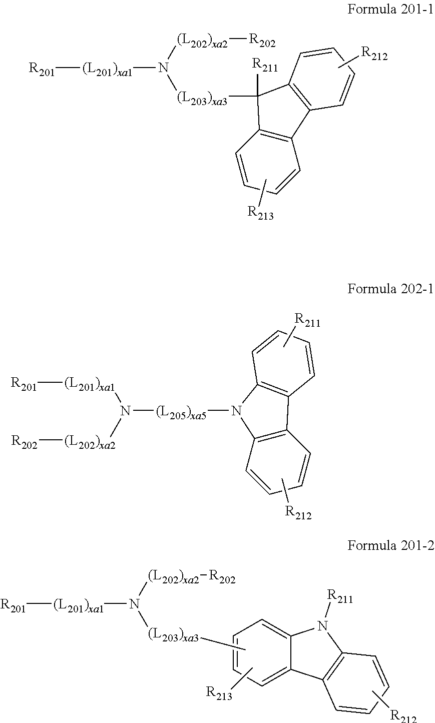

wherein in Formulae 201-1, 202-1, and 201-2, L₂₀₁ to L₂₀₃, L₂₀₅, xa1 to xa3, xa5, R₂₀₁, and R₂₀₂ may respectively be understood by referring to the descriptions of L₂₀₁ to L₂₀₃, L₂₀₅, xa1 to xa3, xa5, R₂₀₁, and R₂₀₂ provided herein, and R₂₁₁ to R₂₁₃ may each independently be hydrogen, deuterium, —F, —Cl, —Br, —I, a hydroxyl group, a cyano group, a nitro group, an amidino group, a hydrazino group, a hydrazono group, a C₁-C₂₀ alkyl group, a C₁-C₂₀ alkoxy group, a phenyl group, a biphenyl group, a terphenyl group, a phenyl group substituted with a C₁-C₁₀ alkyl group, a phenyl group substituted with —F, a naphthyl group, a fluorenyl group, a spiro-bifluorenyl group, a dimethylfluorenyl group, a diphenylfluorenyl group, a triphenylenyl group, a thiophenyl group, a furanyl group, a carbazolyl group, an indolyl group, an isoindolyl group, a benzofuranyl group, a benzothiophenyl group, a dibenzofuranyl group, a dibenzothiophenyl group, a benzocarbazolyl group, a dibenzocarbazolyl group, a dibenzosilolyl group, or a pyridinyl group.

In some embodiments, the hole transport region 12 may include at least one of Compounds HT1 to HT39, but embodiments are not limited thereto:

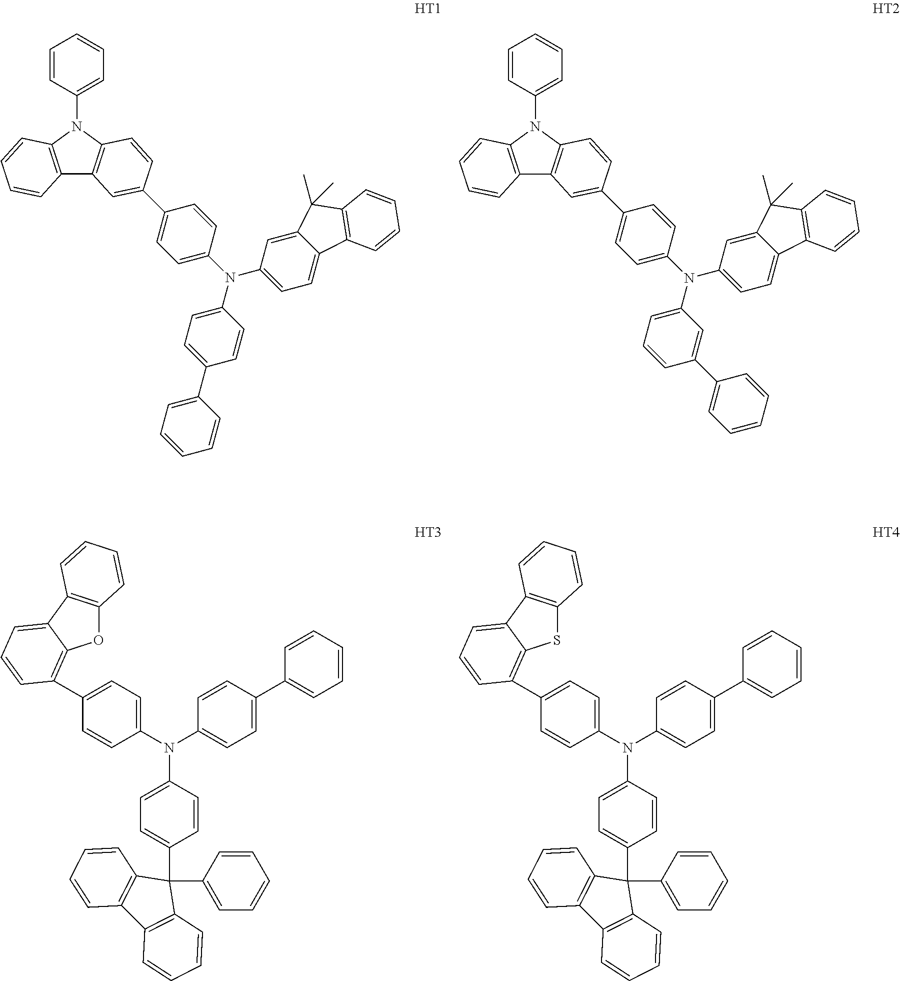 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; CC1(C)c2ccccc2-c2ccc(N(c3ccc(-c4ccc5c(c4)c4ccccc4n5-c4ccccc4)cc3)c3cccc(-c4ccccc4)c3)cc21 ; c1ccc(-c2ccc(N(c3ccc(-c4cccc5c4oc4ccccc45)cc3)c3ccc(C4(c5ccccc5)c5ccccc5-c5ccccc54)cc3)cc2)cc1 ; c1ccc(-c2ccc(N(c3ccc(-c4cccc5c4sc4ccccc45)cc3)c3ccc(C4(c5ccccc5)c5ccccc5-c5ccccc54)cc3)cc2)cc1]

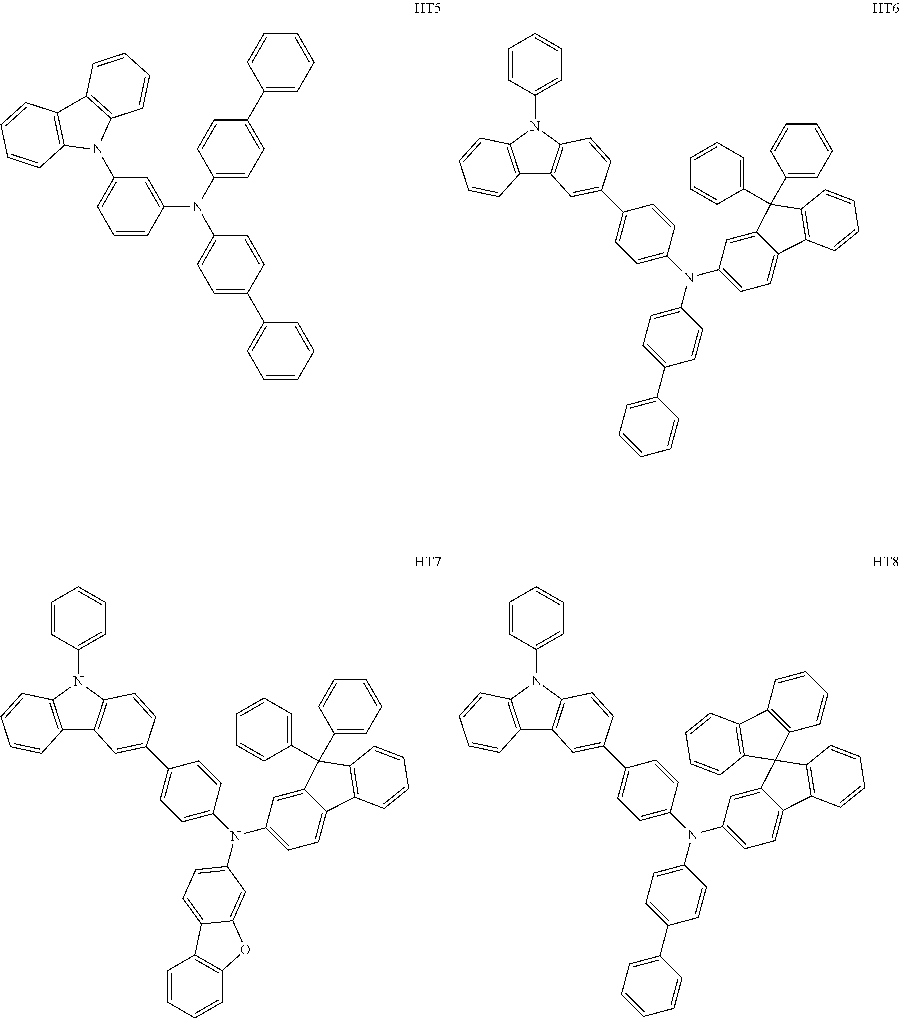 [SMILES: c1ccc(-c2ccc(N(c3ccc(-c4ccccc4)cc3)c3cccc(-n4c5ccccc5c5ccccc54)c3)cc2)cc1 ; c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1 ; c1ccc(-n2c3ccccc3c3cc(-c4ccc(N(c5ccc6c(c5)C(c5ccccc5)(c5ccccc5)c5ccccc5-6)c5ccc6c(c5)oc5ccccc56)cc4)ccc32)cc1 ; c1ccc(-c2ccc(N(c3ccc(-c4ccc5c(c4)c4ccccc4n5-c4ccccc4)cc3)c3ccc4c(c3)C3(c5ccccc5-c5ccccc53)c3ccccc3-4)cc2)cc1]

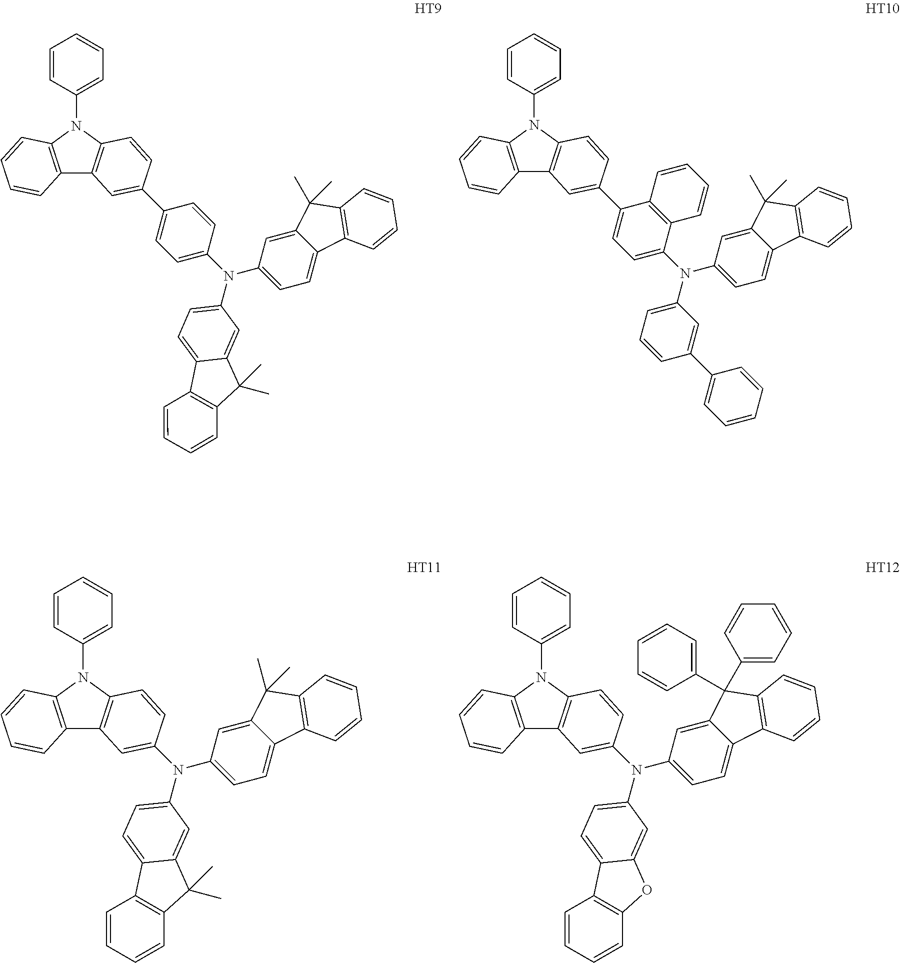 [SMILES: CC1(C)c2ccccc2-c2ccc(N(c3ccc(-c4ccc5c(c4)c4ccccc4n5-c4ccccc4)cc3)c3ccc4c(c3)C(C)(C)c3ccccc3-4)cc21 ; CC1(C)c2ccccc2-c2ccc(N(c3cccc(-c4ccccc4)c3)c3ccc(-c4ccc5c(c4)c4ccccc4n5-c4ccccc4)c4ccccc34)cc21 ; CC1(C)c2ccccc2-c2ccc(N(c3ccc4c(c3)C(C)(C)c3ccccc3-4)c3ccc4c(c3)c3ccccc3n4-c3ccccc3)cc21 ; c1ccc(-n2c3ccccc3c3cc(N(c4ccc5c(c4)C(c4ccccc4)(c4ccccc4)c4ccccc4-5)c4ccc5c(c4)oc4ccccc45)ccc32)cc1]

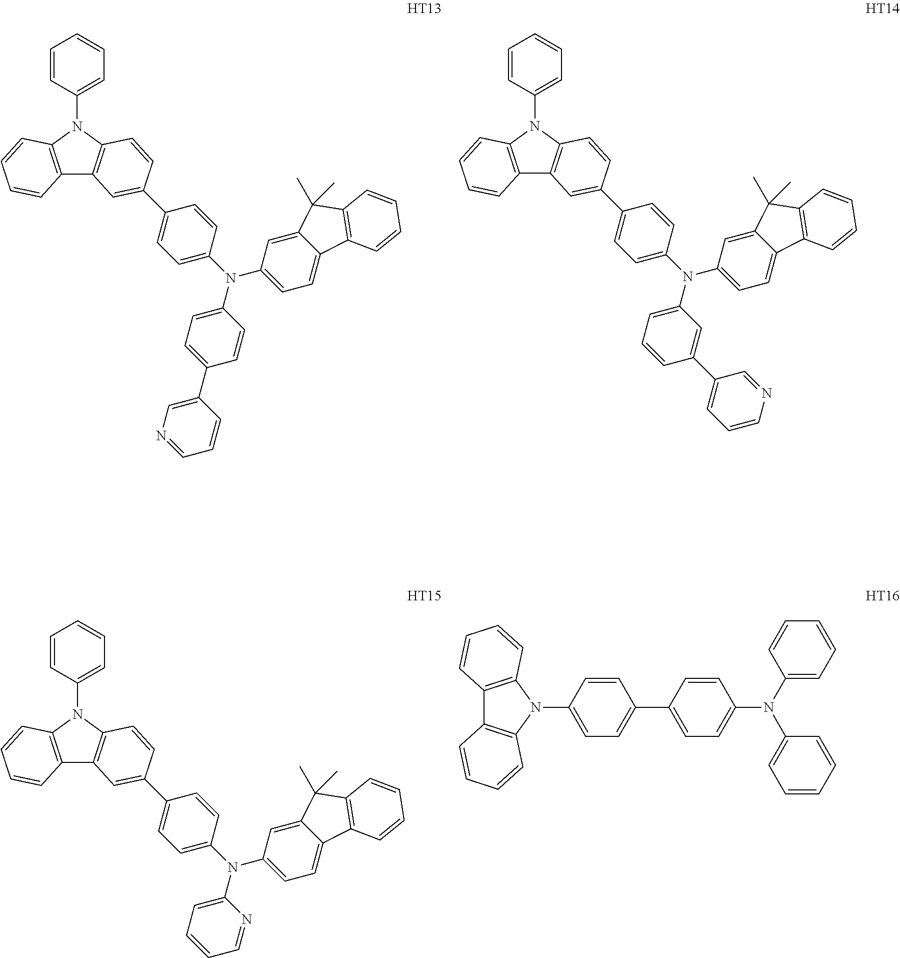 [SMILES: CC1(C)c2ccccc2-c2ccc(N(c3ccc(-c4cccnc4)cc3)c3ccc(-c4ccc5c(c4)c4ccccc4n5-c4ccccc4)cc3)cc21 ; CC1(C)c2ccccc2-c2ccc(N(c3ccc(-c4ccc5c(c4)c4ccccc4n5-c4ccccc4)cc3)c3cccc(-c4cccnc4)c3)cc21 ; CC1(C)c2ccccc2-c2ccc(N(c3ccc(-c4ccc5c(c4)c4ccccc4n5-c4ccccc4)cc3)c3ccccn3)cc21 ; c1ccc(N(c2ccccc2)c2ccc(-c3ccc(-n4c5ccccc5c5ccccc54)cc3)cc2)cc1]

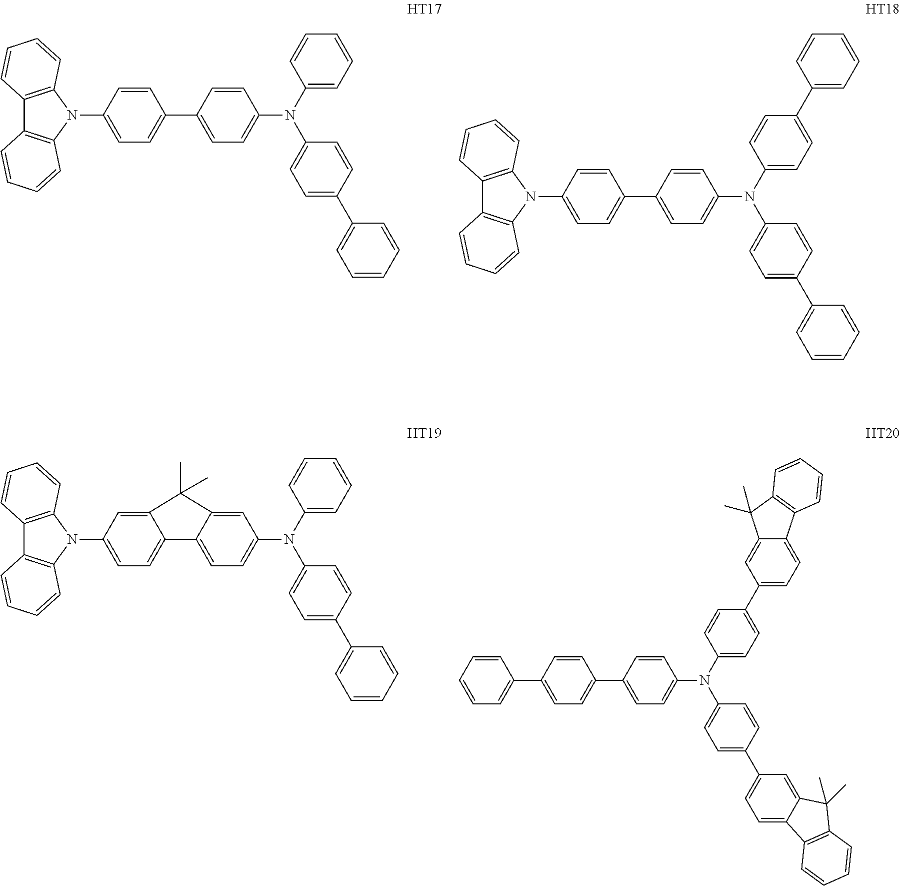 [SMILES: c1ccc(-c2ccc(N(c3ccccc3)c3ccc(-c4ccc(-n5c6ccccc6c6ccccc65)cc4)cc3)cc2)cc1 ; c1ccc(-c2ccc(N(c3ccc(-c4ccccc4)cc3)c3ccc(-c4ccc(-n5c6ccccc6c6ccccc65)cc4)cc3)cc2)cc1 ; CC1(C)c2cc(N(c3ccccc3)c3ccc(-c4ccccc4)cc3)ccc2-c2ccc(-n3c4ccccc4c4ccccc43)cc21 ; CC1(C)c2ccccc2-c2ccc(-c3ccc(N(c4ccc(-c5ccc(-c6ccccc6)cc5)cc4)c4ccc(-c5ccc6c(c5)C(C)(C)c5ccccc5-6)cc4)cc3)cc21]

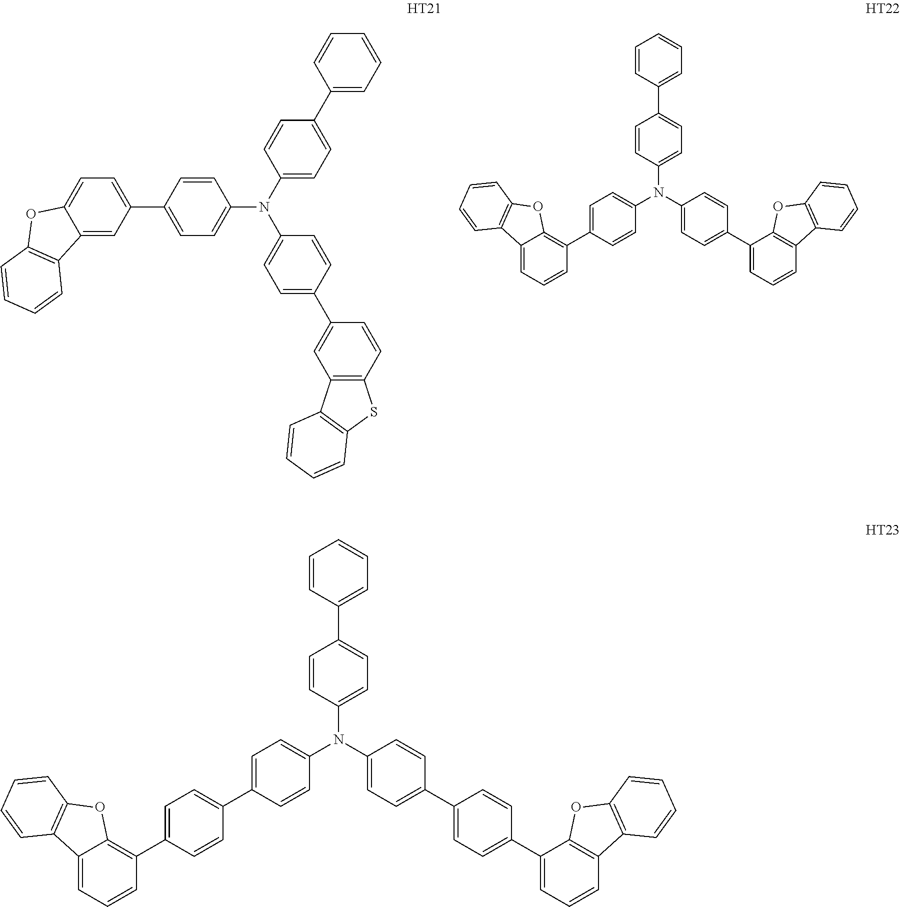 [SMILES: c1ccc(-c2ccc(N(c3ccc(-c4ccc5oc6ccccc6c5c4)cc3)c3ccc(-c4ccc5sc6ccccc6c5c4)cc3)cc2)cc1 ; c1ccc(-c2ccc(N(c3ccc(-c4cccc5c4oc4ccccc45)cc3)c3ccc(-c4cccc5c4oc4ccccc45)cc3)cc2)cc1 ; c1ccc(-c2ccc(N(c3ccc(-c4ccc(-c5cccc6c5oc5ccccc56)cc4)cc3)c3ccc(-c4ccc(-c5cccc6c5oc5ccccc56)cc4)cc3)cc2)cc1]

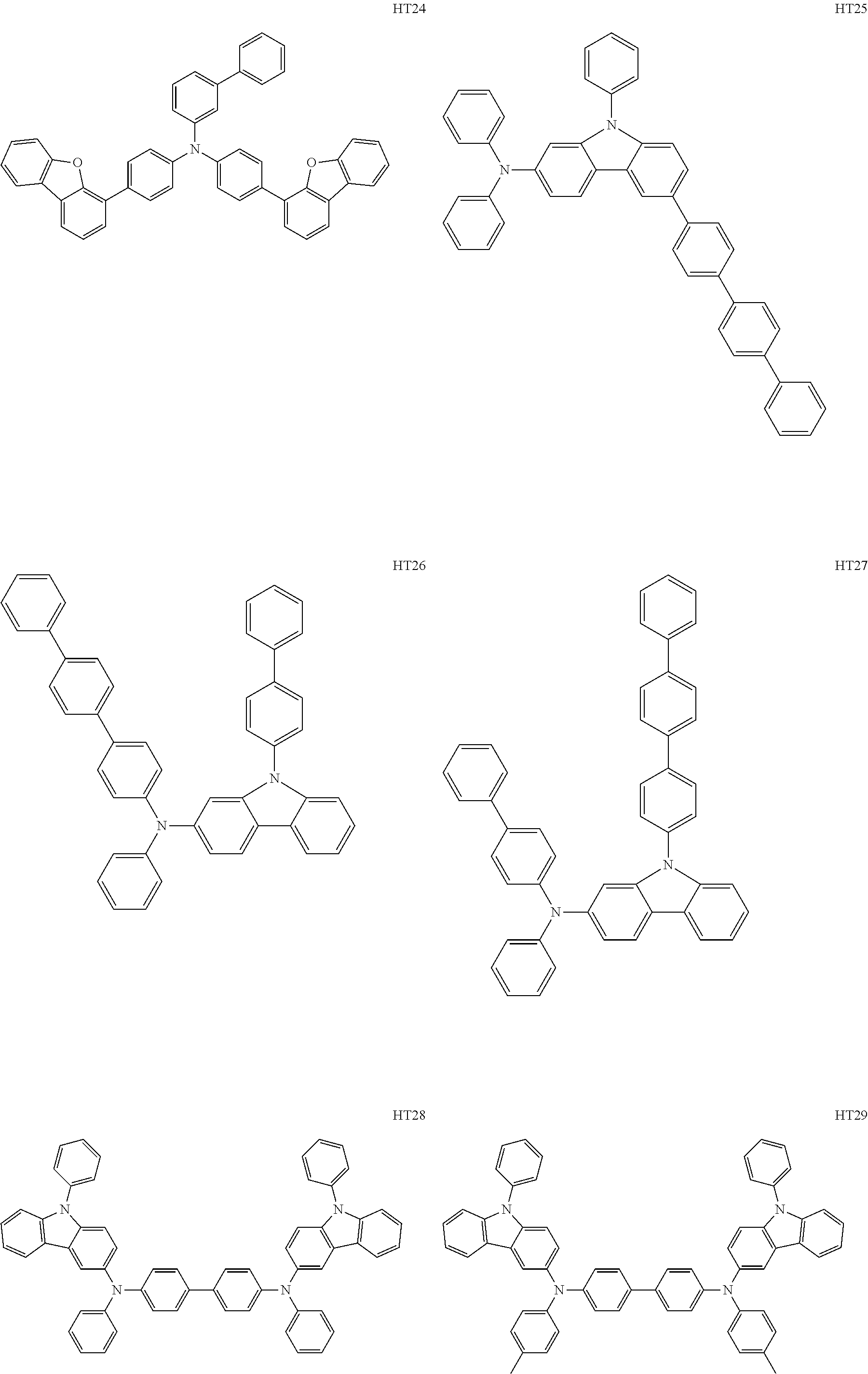

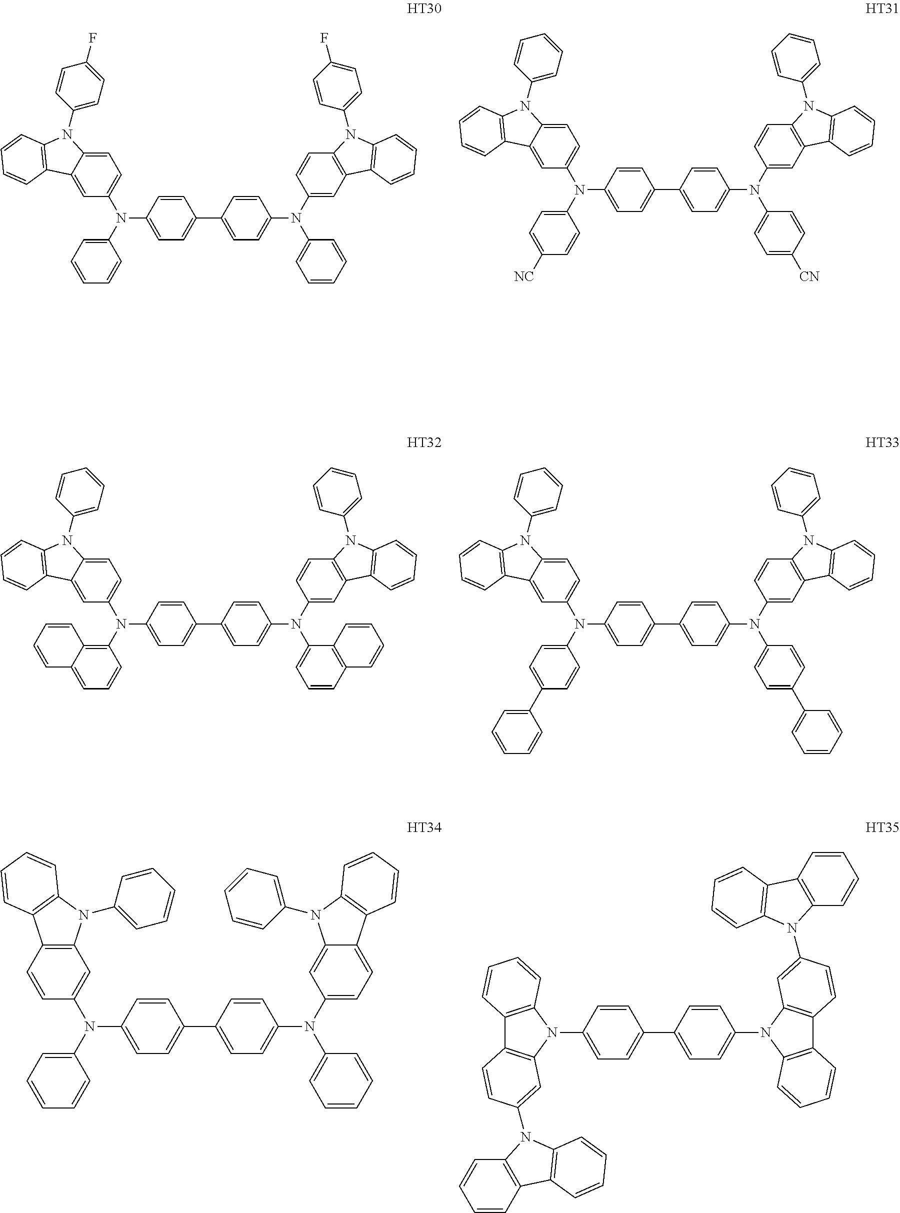

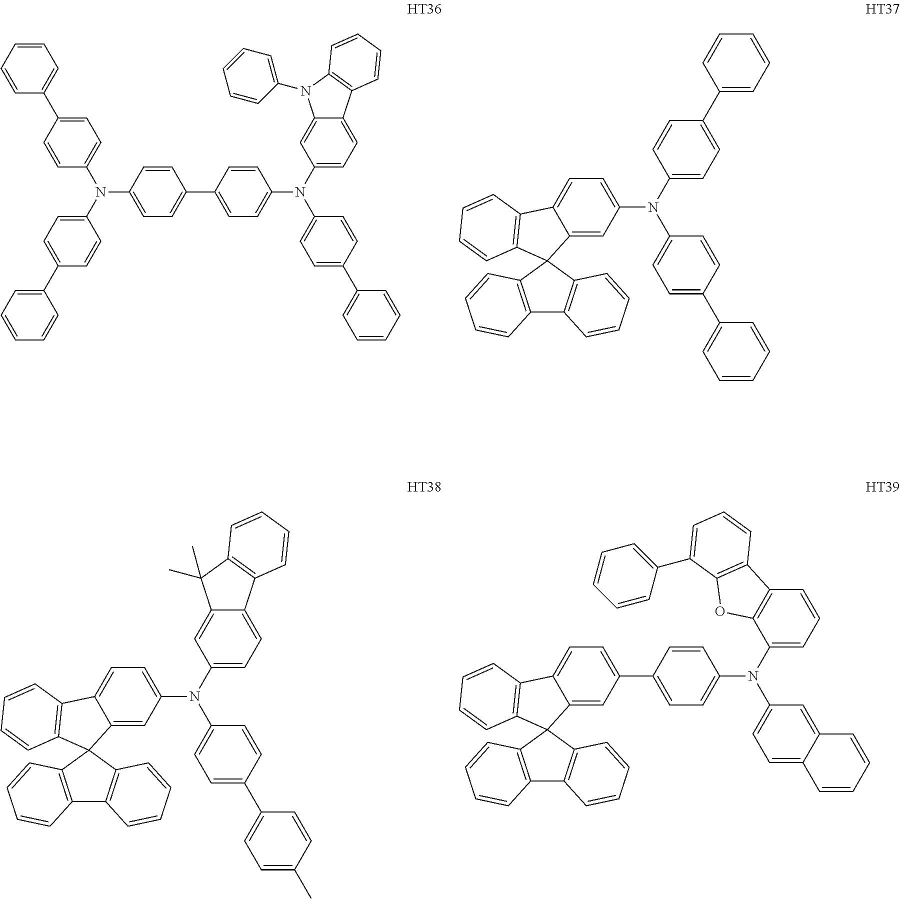 [SMILES: c1ccc(-c2ccc(N(c3ccc(-c4ccccc4)cc3)c3ccc(-c4ccc(N(c5ccc(-c6ccccc6)cc5)c5ccc6c7ccccc7n(-c7ccccc7)c6c5)cc4)cc3)cc2)cc1 ; c1ccc(-c2ccc(N(c3ccc(-c4ccccc4)cc3)c3ccc4c(c3)C3(c5ccccc5-c5ccccc53)c3ccccc3-4)cc2)cc1 ; Cc1ccc(-c2ccc(N(c3ccc4c(c3)C(C)(C)c3ccccc3-4)c3ccc4c(c3)C3(c5ccccc5-c5ccccc53)c3ccccc3-4)cc2)cc1 ; c1ccc(-c2cccc3c2oc2c(N(c4ccc(-c5ccc6c(c5)C5(c7ccccc7-c7ccccc75)c5ccccc5-6)cc4)c4ccc5ccccc5c4)cccc23)cc1]

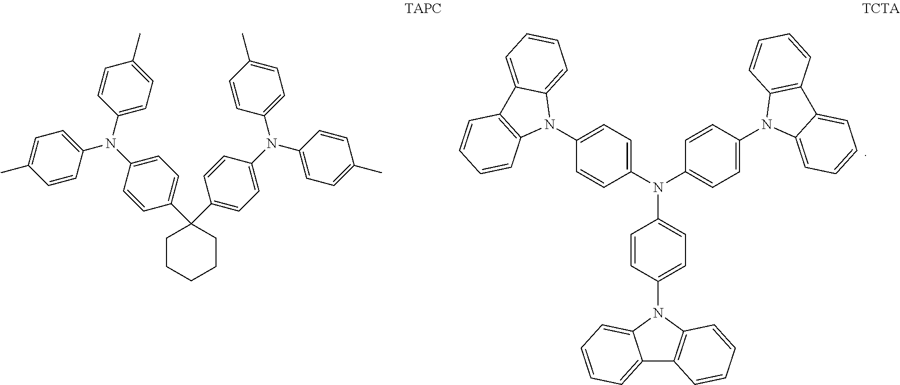

In some embodiments, the hole transport region may include at least one compound of 4,4′-cyclohexylidenebis[N,N-bis(4-methylphenyl)benzenamine](TAPC), tris(4-carbazoyl-9-ylphenyl)amine (TCTA), or any combination thereof.

The hole transport region 12 of the organic light-emitting device 10 may further include a p-dopant. When the hole transport region 12 further includes a p-dopant, the hole transport region 12 may have a structure including a matrix (for example, at least one compound represented by Formulae 201 to 205) and a p-dopant included in the matrix. The p-dopant may be homogeneously or non-homogeneously doped in the hole transport region 12.

In some embodiments, a LUMO energy level of the p-dopant may be about −3.5 eV or less.

The p-dopant may include at least one of a quinone derivative, a metal oxide, a cyano group-containing compound, or any combination thereof, but embodiments are not limited thereto.

In some embodiments, the p-dopant may include:

a quinone derivative such as tetracyanoquinodimethane (TCNQ), 2,3,5,6-tetrafluoro-7,7,8,8-tetracyanoquinodimethane (F4-TCNQ), or F6-TCNNQ;

a metal oxide such as tungsten oxide or molybdenum oxide;

1,4,5,8,9,12-hexaazatriphenylene-hexacarbonitrile (HAT-CN); and

a compound represented by Formula 221, but embodiments are not limited thereto:

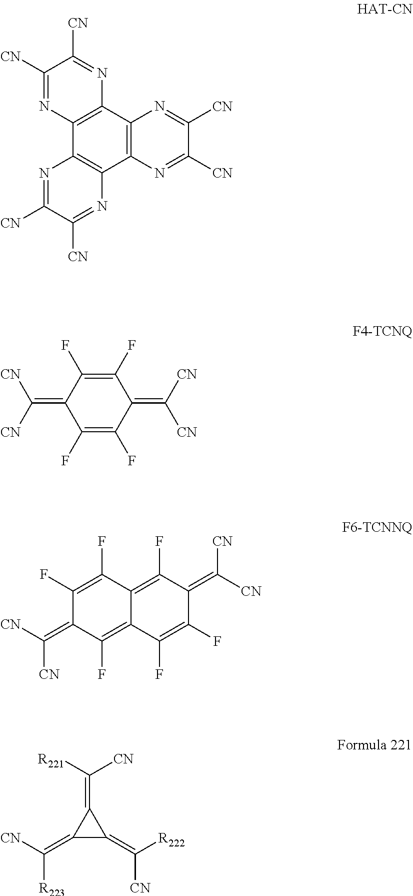

wherein, in Formula 221,

R₂₂₁ to R₂₂₃ may each independently be a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₁-C₆₀ heteroaryl group, a substituted or unsubstituted monovalent aromatic condensed polycyclic group, a substituted or unsubstituted monovalent aromatic condensed heteropolycyclic group, a substituted or unsubstituted monovalent non-aromatic condensed polycyclic group, or a substituted or unsubstituted monovalent non-aromatic condensed heteropolycyclic group, wherein at least one R₂₂₁ to R₂₂₃ may include at least one a cyano group, —F, —Cl, —Br, —I, a C₁-C₂₀ alkyl group substituted with —F, a C₁-C₂₀ alkyl group substituted with —Cl, a C₁-C₂₀ alkyl group substituted with —Br, a C₁-C₂₀ alkyl group substituted with —I, or any combination thereof.

A thickness of the hole transport region 12 may be in a range of about 100 Angstroms (Å) to about 10,000 Å, for example, about 400 Å to about 2,000 Å, and a thickness of the emission layer 15 may be in a range of about 100 Å to about 3,000 Å, for example, about 300 Å to about 1,000 Å. When the thicknesses of the hole transport region 12 and the emission layer 15 are within any of these ranges, satisfactory hole transporting characteristics and/or luminescence characteristics may be obtained without a substantial increase in driving voltage.

### Electron Transport Region 17

In the organic light-emitting device 10, the electron transport region 17 may be between the emission layer 15 and the second electrode 19.

The electron transport region 17 may have a single-layered structure or a multi-layered structure.

For example, the electron transport region 17 may have a structure of electron transport layer, a structure of electron transport layer/electron injection layer, a structure of buffer layer/electron transport layer, a structure of hole blocking layer/electron transport layer, a structure of buffer layer/electron transport layer/electron injection layer, or a structure of hole blocking layer/electron transport layer/electron injection layer, but embodiments are not limited thereto. The electron transport region 17 may include an electron control layer.

The electron transport region 17 may include a known electron transport material.

The electron transport region 17 (for example, the buffer layer, the hole blocking layer, the electron control layer, or the electron transport layer in the electron transport region 17) may include a metal-free compound including at least one π electron-depleted nitrogen-containing cyclic group. The π electron-depleted nitrogen-containing cyclic group may be understood by referring to the description for those provided herein.

In some embodiments, the electron transport region may include a compound represented by Formula 601:

[Ar₆₀₁]ₓₑ₁₁—[(L₆₀₁)ₓₑ₁—R₆₀₁]ₓₑ₂₁ Formula 601

wherein, in Formula 601,

Ar₆₀₁ and L₆₀₁ may each independently be a substituted or unsubstituted C₅-C₆₀ carbocyclic group or a substituted or unsubstituted C₁-C₆₀ heterocyclic group,

xe11 may be 1,2, or 3,

xe1 may be an integer from 0 to 5,

R₆₀₁ may be a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent aromatic condensed polycyclic group, a substituted or unsubstituted monovalent aromatic condensed heteropolycyclic group, a substituted or unsubstituted monovalent non-aromatic condensed polycyclic group, a substituted or unsubstituted monovalent non-aromatic condensed heteropolycyclic group, —Si(Q₆₀₁)(Q₆₀₂)(Q₆₀₃), —C(═O)(Q₆₀₁), —S(═O)₂(Q₆₀₁), or —P(═O)(Q₆₀₁)(Q₆₀₂),

Q₆₀₁ to Q₆₀₃ may each independently be a C₁-C₁₀ alkyl group, a C₁-C₁₀ alkoxy group, a phenyl group, a biphenyl group, a terphenyl group, or a naphthyl group, and

xe21 may be an integer from 1 to 5.

In some embodiments, at least one Ar₆₀₁(s) in the number of xe11 and R₆₀₁(s) in the number of xe21 may include the π electron-depleted nitrogen-containing cyclic group.

In some embodiments, in Formula 601, ring Ar₆₀₁ and L₆₀₁ may each independently be a benzene group, a naphthalene group, a fluorene group, a spiro-bifluorene group, a benzofluorene group, a dibenzofluorene group, a phenalene group, a phenanthrene group, an anthracene group, a fluoranthene group, a triphenylene group, a pyrene group, a chrysene group, a naphthacene group, a picene group, a perylene group, a pentaphene group, an indenoanthracene group, a dibenzofuran group, a dibenzothiophene group, a carbazole group, an imidazole group, a pyrazole group, a thiazole group, an isothiazole group, an oxazole group, an isoxazole group, a pyridine group, a pyrazine group, a pyrimidine group, a pyridazine group, an indazole group, a purine group, a quinoline group, an isoquinoline group, a benzoquinoline group, a phthalazine group, a naphthyridine group, a quinoxaline group, a quinazoline group, a cinnoline group, a phenanthridine group, an acridine group, a phenanthroline group, a phenazine group, a benzimidazole group, an isobenzothiazole group, a benzoxazole group, an isobenzoxazole group, a triazole group, a tetrazole group, an oxadiazole group, a triazine group, a thiadiazole group, an imidazopyridine group, an imidazopyrimidine group, or an azacarbazole group, each unsubstituted or substituted with at least one deuterium, —F, —Cl, —Br, —I, a hydroxyl group, a cyano group, a nitro group, an amidino group, a hydrazino group, a hydrazono group, a C₁-C₂₀ alkyl group, a C₁-C₂₀ alkoxy group, a phenyl group, a biphenyl group, a terphenyl group, a naphthyl group, —Si(Q₃₁)(Q₃₂)(Q₃₃), —S(═O)₂(Q₃₁), —P(═O)(Q₃₁)(Q₃₂), or any combination thereof,

wherein Q₃₁ to Q₃₃ may each independently be a C₁-C₁₀ alkyl group, a C₁-C₁₀ alkoxy group, a phenyl group, a biphenyl group, a terphenyl group, or a naphthyl group.

When xe11 in Formula 601 is 2 or greater, at least two Ar₆₀₁(s) may be bound via a single bond.

In one or more embodiments, Ar₆₀₁ in Formula 601 may be an anthracene group.

In some embodiments, the compound represented by Formula 601 may be represented by Formula 601-1:

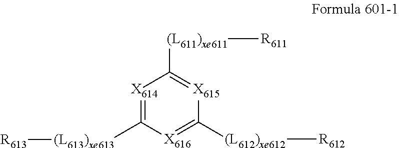

wherein, in Formula 601-1,

X₆₁₄ may be N or C(R₆₁₄), X₆₁₅ may be N or C(R₆₁₅), X₆₁₆ may be N or C(R₆₁₆), at least one X₆₁₄ to X₆₁₆ may be N,

L₆₁₁ to L₆₁₃ may each independently be understood by referring to the description of L₆₀₁ provided herein,

xe611 to xe613 may each independently be understood by referring to the description of xe1 provided herein,

R₆₁₁ to R₆₁₃ may each independently be understood by referring to the description of R₆₀₁ provided herein, and

R₆₁₄ to R₆₁₆ may each independently be hydrogen, deuterium, —F, —Cl, —Br, —I, a hydroxyl group, a cyano group, a nitro group, an amidino group, a hydrazino group, a hydrazono group, a C₁-C₂₀ alkyl group, a C₁-C₂₀ alkoxy group, a phenyl group, a biphenyl group, a terphenyl group, or a naphthyl group.

In one or more embodiments, in Formulae 601 and 601-1, xe1 and xe611 to xe613 may each independently be 0, 1, or 2.

In one or more embodiments, in Formulae 601 and 601-1, R₆₀₁ and R₆₁₁ to R₆₁₃ may each independently be: a phenyl group, a biphenyl group, a terphenyl group, a naphthyl group, a fluorenyl group, a spiro-bifluorenyl group, a benzofluorenyl group, a dibenzofluorenyl group, a phenanthrenyl group, an anthracenyl group, a fluoranthenyl group, a triphenylenyl group, a pyrenyl group, a chrysenyl group, a perylenyl group, a pentaphenyl group, a hexacenyl group, a pentacenyl group, a thiophenyl group, a furanyl group, a carbazolyl group, an indolyl group, an isoindolyl group, a benzofuranyl group, a benzothiophenyl group, a dibenzofuranyl group, a dibenzothiophenyl group, a benzocarbazolyl group, a dibenzocarbazolyl group, a dibenzosilolyl group, a pyridinyl group, an imidazolyl group, a pyrazolyl group, a thiazolyl group, an isothiazolyl group, an oxazolyl group, an isoxazolyl group, a thiadiazolyl group, an oxadiazolyl group, a pyrazinyl group, a pyrimidinyl group, a pyridazinyl group, a triazinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a phenanthridinyl group, an acridinyl group, a phenanthrolinyl group, a phenazinyl group, a benzimidazolyl group, an isobenzothiazolyl group, a benzoxazolyl group, an isobenzoxazolyl group, a triazolyl group, a tetrazolyl group, an imidazopyridinyl group, an imidazopyrimidinyl group, or an azacarbazolyl group, each unsubstituted or substituted with at least one deuterium, —F, —Cl, —Br, —I, a hydroxyl group, a cyano group, a nitro group, an amidino group, a hydrazino group, a hydrazono group, a C₁-C₂₀ alkyl group, a C₁-C₂₀ alkoxy group, a phenyl group, a biphenyl group, a terphenyl group, a naphthyl group, a fluorenyl group, a spiro-bifluorenyl group, a benzofluorenyl group, a dibenzofluorenyl group, a phenanthrenyl group, an anthracenyl group, a fluoranthenyl group, a triphenylenyl group, a pyrenyl group, a chrysenyl group, a perylenyl group, a pentaphenyl group, a hexacenyl group, a pentacenyl group, a thiophenyl group, a furanyl group, a carbazolyl group, an indolyl group, an isoindolyl group, a benzofuranyl group, a benzothiophenyl group, a dibenzofuranyl group, a dibenzothiophenyl group, a benzocarbazolyl group, a dibenzocarbazolyl group, a dibenzosilolyl group, a pyridinyl group, an imidazolyl group, a pyrazolyl group, a thiazolyl group, an isothiazolyl group, an oxazolyl group, an isoxazolyl group, a thiadiazolyl group, an oxadiazolyl group, a pyrazinyl group, a pyrimidinyl group, a pyridazinyl group, a triazinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a phenanthridinyl group, an acridinyl group, a phenanthrolinyl group, a phenazinyl group, a benzimidazolyl group, an isobenzothiazolyl group, a benzoxazolyl group, an isobenzoxazolyl group, a triazolyl group, a tetrazolyl group, an imidazopyridinyl group, an imidazopyrimidinyl group, an azacarbazolyl group, or any combination thereof; or

—S(═O)₂(Q₆₀₁) or —P(═O)(Q₆₀₁)(Q₆₀₂),

wherein Q₆₀₁ and Q₆₀₂ may respectively be understood by referring to the descriptions of Q₆₀₁ and Q₆₀₂ provided herein.

The electron transport region may include at least one compound of Compounds ET1 to ET36, but embodiments are not limited thereto:

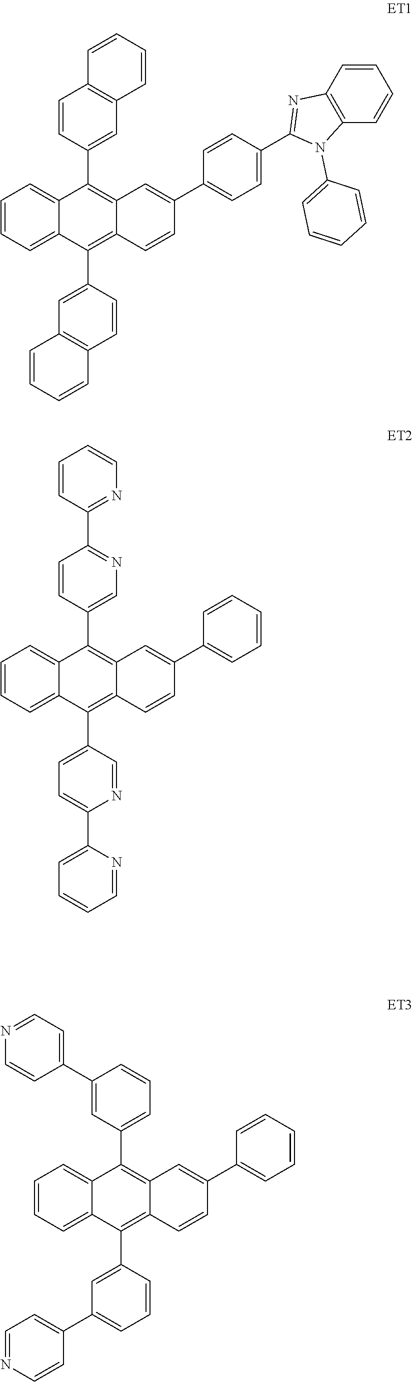 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc(-c2ccc3c(-c4ccc(-c5ccccn5)nc4)c4ccccc4c(-c4ccc(-c5ccccn5)nc4)c3c2)cc1 ; c1ccc(-c2ccc3c(-c4cccc(-c5ccncc5)c4)c4ccccc4c(-c4cccc(-c5ccncc5)c4)c3c2)cc1]

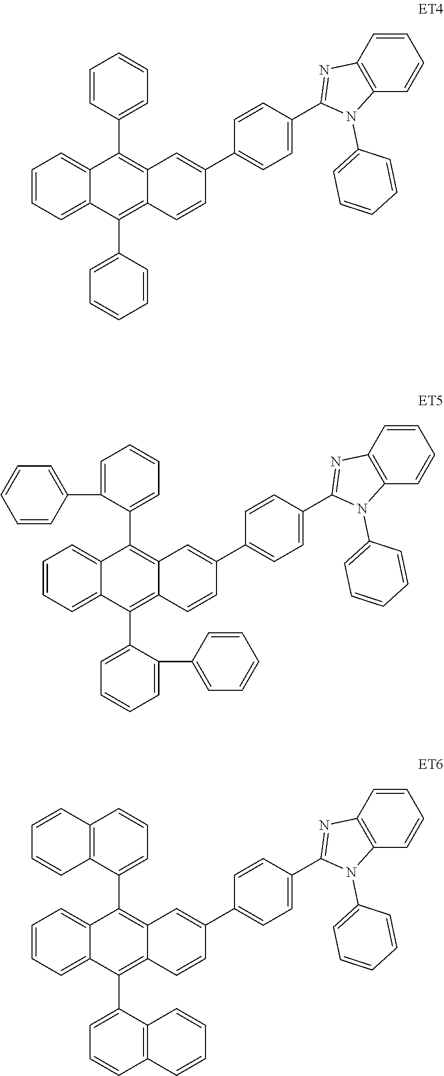 [SMILES: c1ccc(-c2c3ccccc3c(-c3ccccc3)c3cc(-c4ccc(-c5nc6ccccc6n5-c5ccccc5)cc4)ccc23)cc1 ; c1ccc(-c2ccccc2-c2c3ccccc3c(-c3ccccc3-c3ccccc3)c3cc(-c4ccc(-c5nc6ccccc6n5-c5ccccc5)cc4)ccc23)cc1 ; c1ccc(-n2c(-c3ccc(-c4ccc5c(-c6cccc7ccccc67)c6ccccc6c(-c6cccc7ccccc67)c5c4)cc3)nc3ccccc32)cc1]

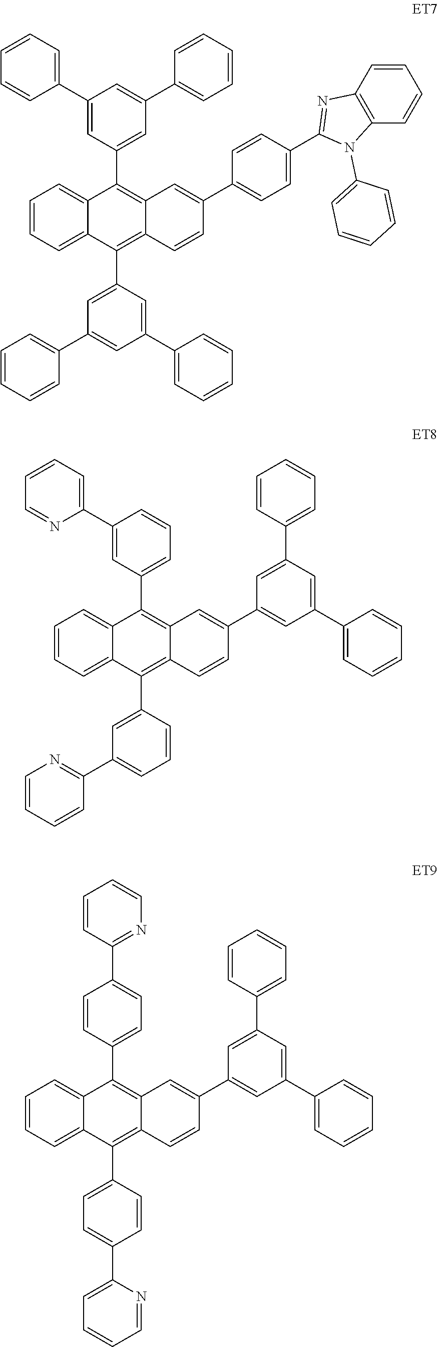 [SMILES: c1ccc(-c2cc(-c3ccccc3)cc(-c3c4ccccc4c(-c4cc(-c5ccccc5)cc(-c5ccccc5)c4)c4cc(-c5ccc(-c6nc7ccccc7n6-c6ccccc6)cc5)ccc34)c2)cc1 ; c1ccc(-c2cc(-c3ccccc3)cc(-c3ccc4c(-c5cccc(-c6ccccn6)c5)c5ccccc5c(-c5cccc(-c6ccccn6)c5)c4c3)c2)cc1 ; c1ccc(-c2cc(-c3ccccc3)cc(-c3ccc4c(-c5ccc(-c6ccccn6)cc5)c5ccccc5c(-c5ccc(-c6ccccn6)cc5)c4c3)c2)cc1]

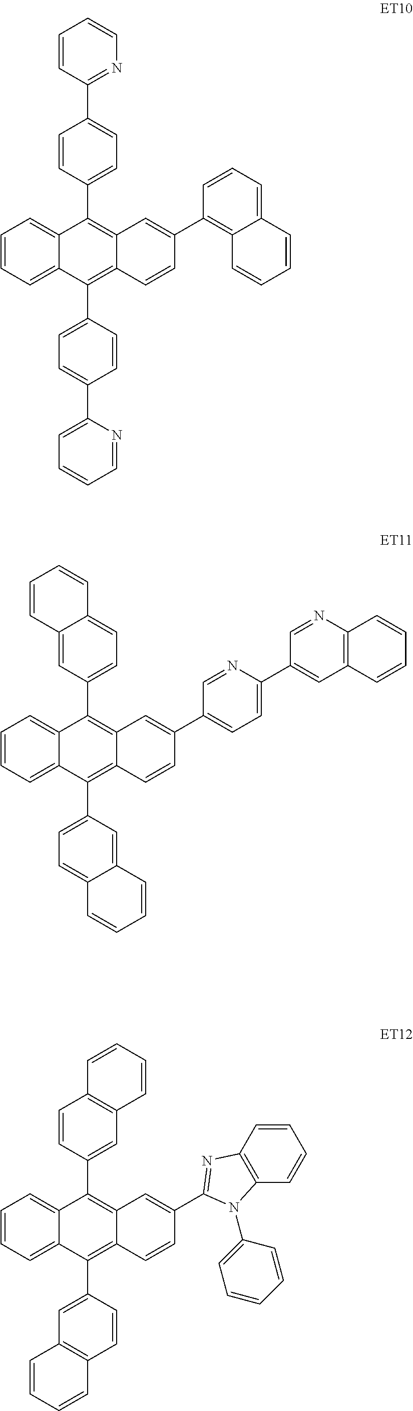 [SMILES: c1ccc(-c2ccc(-c3c4ccccc4c(-c4ccc(-c5ccccn5)cc4)c4cc(-c5cccc6ccccc56)ccc34)cc2)nc1 ; c1ccc2cc(-c3c4ccccc4c(-c4ccc5ccccc5c4)c4cc(-c5ccc(-c6cnc7ccccc7c6)nc5)ccc34)ccc2c1 ; c1ccc(-n2c(-c3ccc4c(-c5ccc6ccccc6c5)c5ccccc5c(-c5ccc6ccccc6c5)c4c3)nc3ccccc32)cc1]

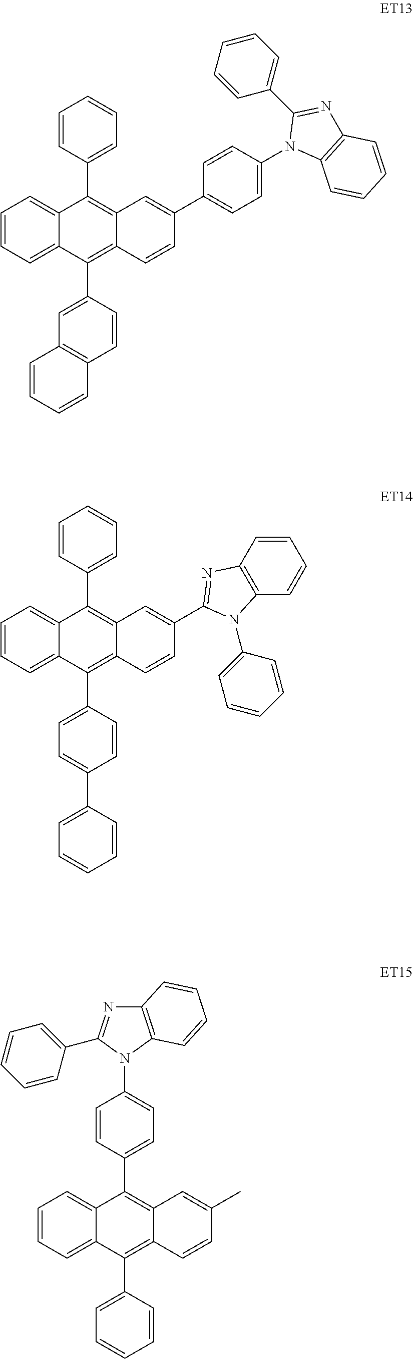 [SMILES: c1ccc(-c2c3ccccc3c(-c3ccc4ccccc4c3)c3ccc(-c4ccc(-n5c(-c6ccccc6)nc6ccccc65)cc4)cc23)cc1 ; c1ccc(-c2ccc(-c3c4ccccc4c(-c4ccccc4)c4cc(-c5nc6ccccc6n5-c5ccccc5)ccc34)cc2)cc1 ; Cc1ccc2c(-c3ccccc3)c3ccccc3c(-c3ccc(-n4c(-c5ccccc5)nc5ccccc54)cc3)c2c1]

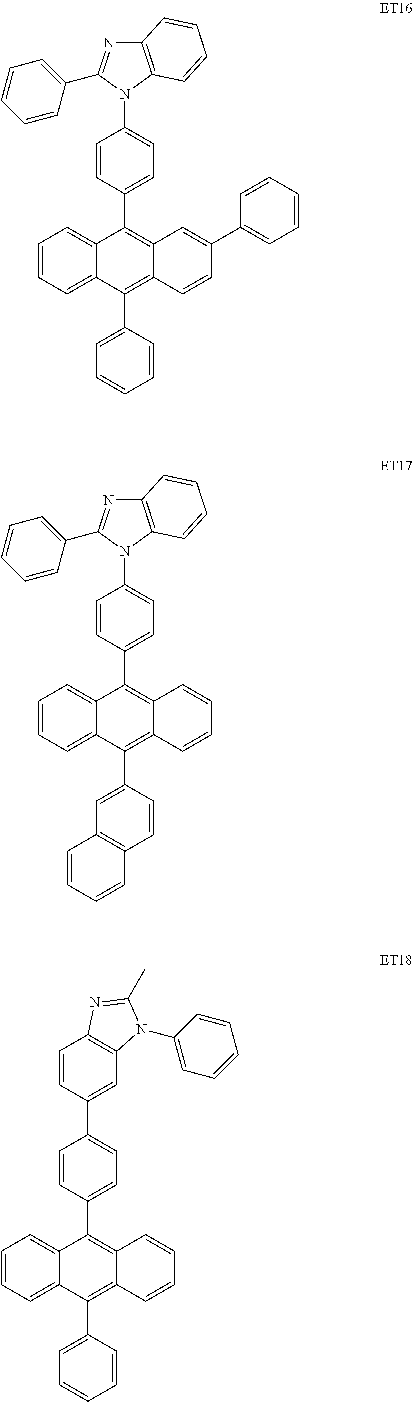 [SMILES: c1ccc(-c2ccc3c(-c4ccccc4)c4ccccc4c(-c4ccc(-n5c(-c6ccccc6)nc6ccccc65)cc4)c3c2)cc1 ; c1ccc(-c2nc3ccccc3n2-c2ccc(-c3c4ccccc4c(-c4ccc5ccccc5c4)c4ccccc34)cc2)cc1 ; Cc1nc2ccc(-c3ccc(-c4c5ccccc5c(-c5ccccc5)c5ccccc45)cc3)cc2n1-c1ccccc1]

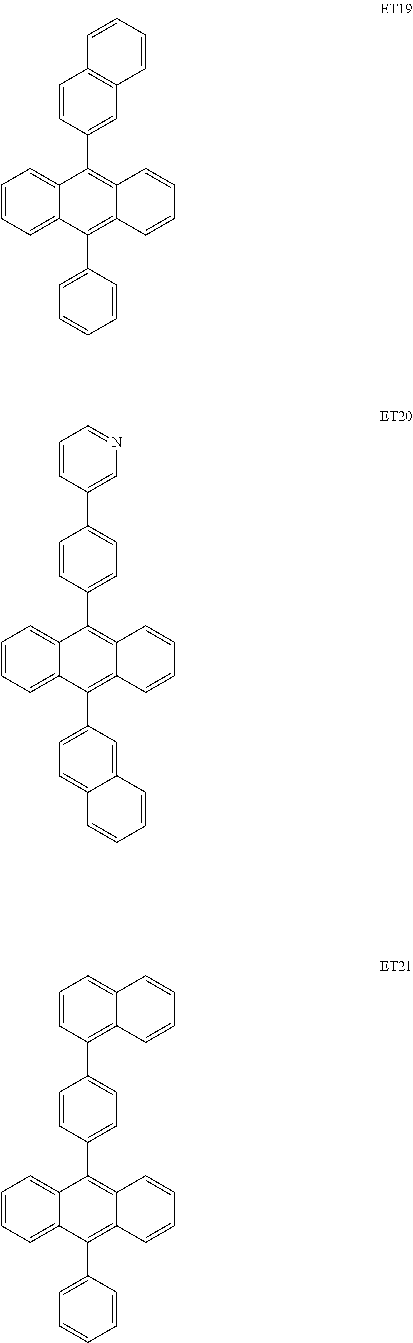

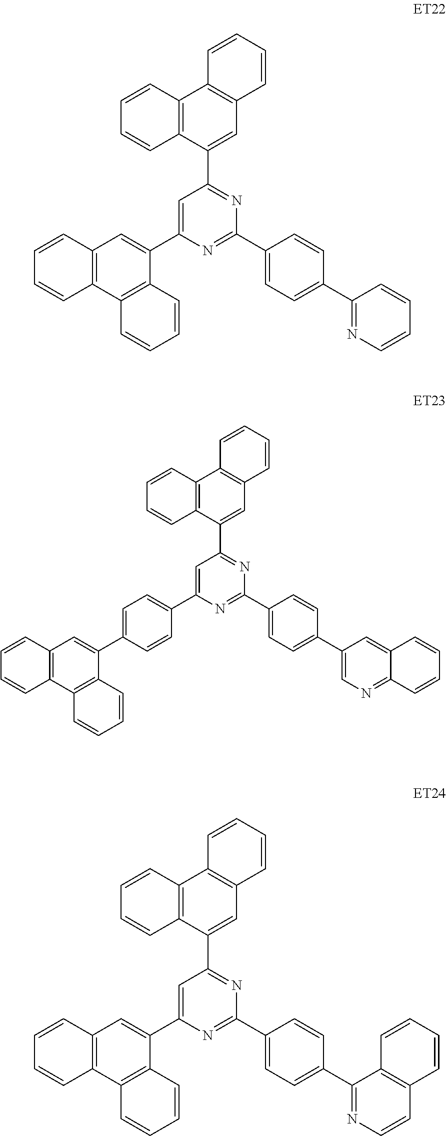 [SMILES: c1ccc(-c2ccc(-c3nc(-c4cc5ccccc5c5ccccc45)cc(-c4cc5ccccc5c5ccccc45)n3)cc2)nc1 ; c1ccc2ncc(-c3ccc(-c4nc(-c5ccc(-c6cc7ccccc7c7ccccc67)cc5)cc(-c5cc6ccccc6c6ccccc56)n4)cc3)cc2c1 ; c1ccc2c(-c3ccc(-c4nc(-c5cc6ccccc6c6ccccc56)cc(-c5cc6ccccc6c6ccccc56)n4)cc3)nccc2c1]

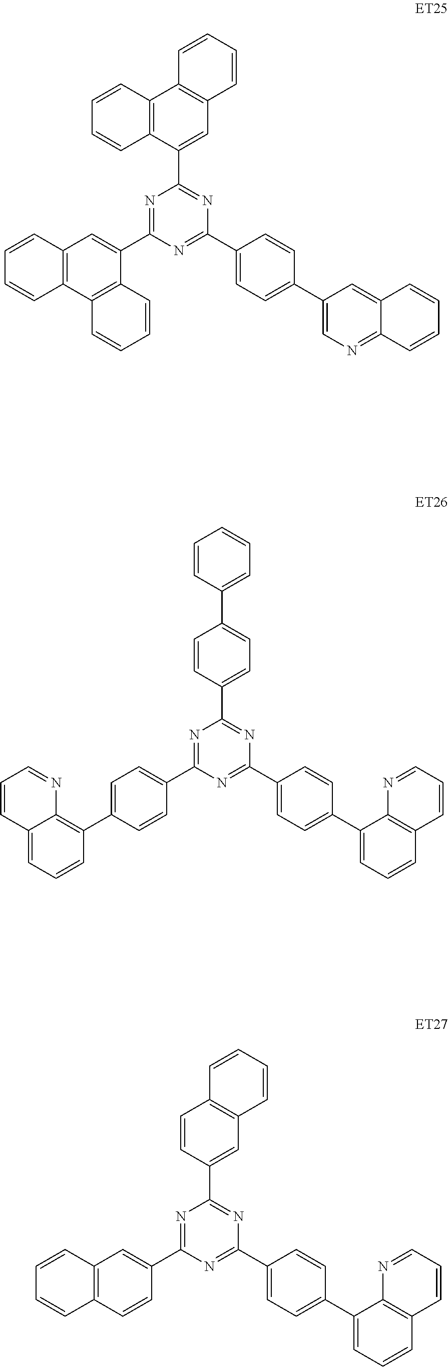 [SMILES: c1ccc2ncc(-c3ccc(-c4nc(-c5cc6ccccc6c6ccccc56)nc(-c5cc6ccccc6c6ccccc56)n4)cc3)cc2c1 ; c1ccc(-c2ccc(-c3nc(-c4ccc(-c5cccc6cccnc56)cc4)nc(-c4ccc(-c5cccc6cccnc56)cc4)n3)cc2)cc1 ; c1ccc2cc(-c3nc(-c4ccc(-c5cccc6cccnc56)cc4)nc(-c4ccc5ccccc5c4)n3)ccc2c1]

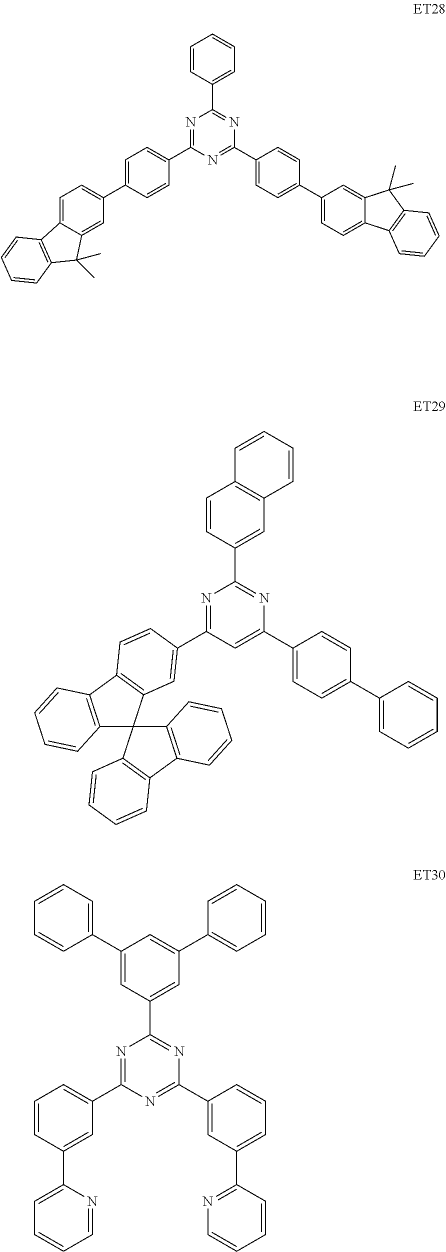 [SMILES: CC1(C)c2ccccc2-c2ccc(-c3ccc(-c4nc(-c5ccccc5)nc(-c5ccc(-c6ccc7c(c6)C(C)(C)c6ccccc6-7)cc5)n4)cc3)cc21 ; c1ccc(-c2ccc(-c3cc(-c4ccc5c(c4)C4(c6ccccc6-c6ccccc64)c4ccccc4-5)nc(-c4ccc5ccccc5c4)n3)cc2)cc1 ; c1ccc(-c2cc(-c3ccccc3)cc(-c3nc(-c4cccc(-c5ccccn5)c4)nc(-c4cccc(-c5ccccn5)c4)n3)c2)cc1]

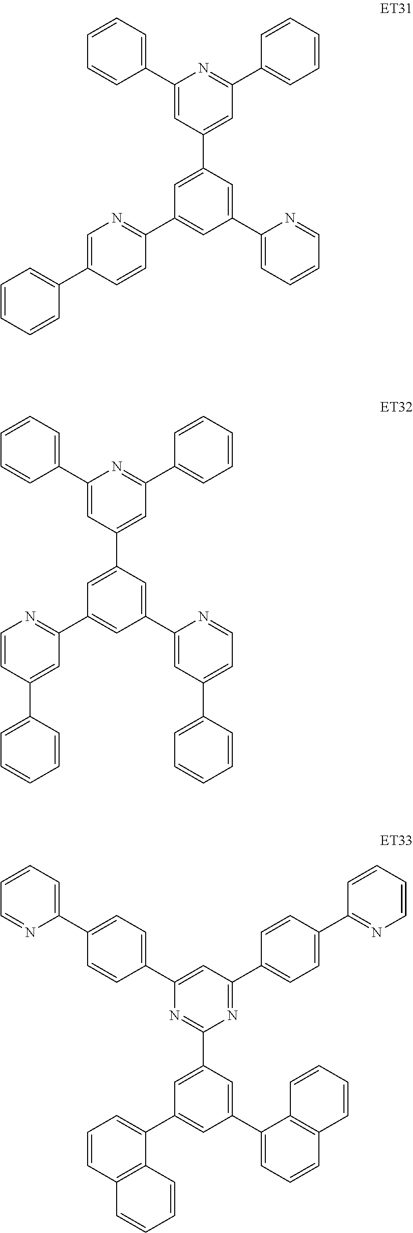 [SMILES: c1ccc(-c2ccc(-c3cc(-c4cc(-c5ccccc5)nc(-c5ccccc5)c4)cc(-c4ccccn4)c3)nc2)cc1 ; c1ccc(-c2ccnc(-c3cc(-c4cc(-c5ccccc5)nc(-c5ccccc5)c4)cc(-c4cc(-c5ccccc5)ccn4)c3)c2)cc1 ; c1ccc(-c2ccc(-c3cc(-c4ccc(-c5ccccn5)cc4)nc(-c4cc(-c5cccc6ccccc56)cc(-c5cccc6ccccc56)c4)n3)cc2)nc1]

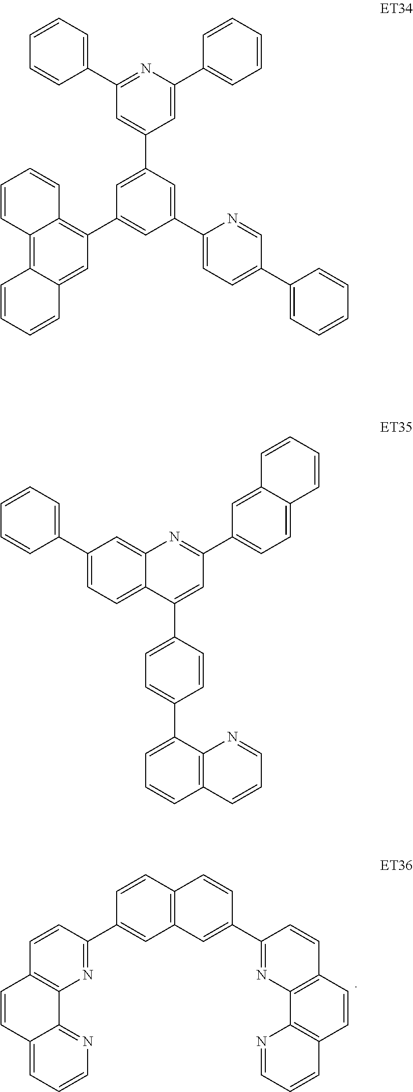 [SMILES: c1ccc(-c2ccc(-c3cc(-c4cc(-c5ccccc5)nc(-c5ccccc5)c4)cc(-c4cc5ccccc5c5ccccc45)c3)nc2)cc1 ; c1ccc(-c2ccc3c(-c4ccc(-c5cccc6cccnc56)cc4)cc(-c4ccc5ccccc5c4)nc3c2)cc1 ; c1cnc2c(c1)ccc1ccc(-c3ccc4ccc(-c5ccc6ccc7cccnc7c6n5)cc4c3)nc12]

In some embodiments, the electron transport region may include at least one compound of 2,9-dimethyl-4,7-diphenyl-1,10-phenanthroline (BCP), 4,7-diphenyl-1,10-phenanthroline (Bphen), Alq3, BAIq, 3-(biphenyl-4-yl)-5-(4-tert-butylphenyl)-4-phenyl-4H-1,2,4-triazole (TAZ), 2,2′,2″-(1,3,5-benzinetriyl)-tris(1-phenyl-1-H-benzimidazole) (TPBi), NTAZ, or any combinations thereof:

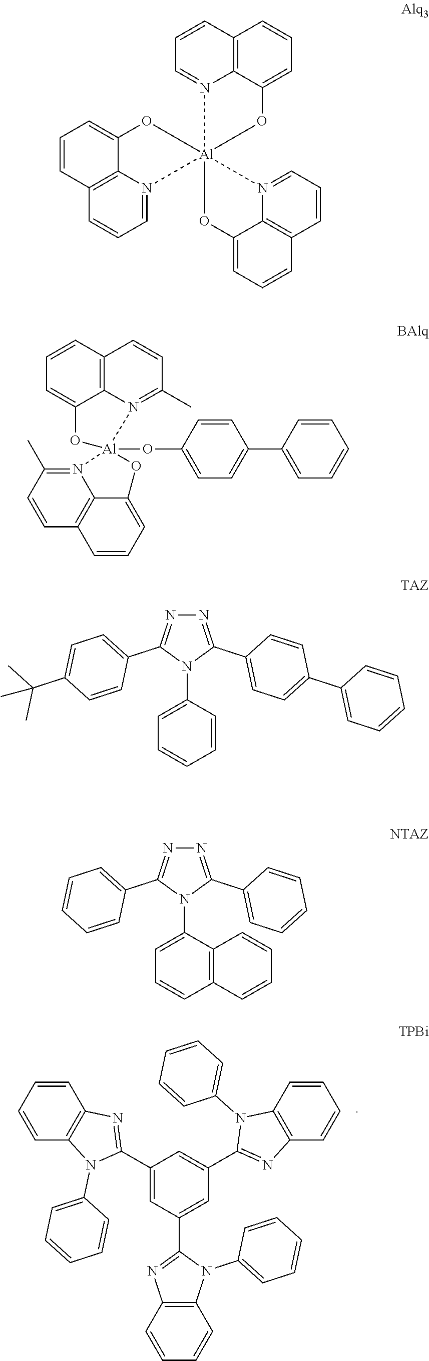

The thicknesses of the buffer layer, the hole blocking layer, or the electron control layer may each independently be in a range of about 20 Å to about 1,000 Å, and in some embodiments, about 30 Å to about 300 Å. When the thicknesses of the buffer layer, the hole blocking layer or the electron control layer are within any of these ranges, excellent hole blocking characteristics or excellent electron controlling characteristics may be obtained without a substantial increase in driving voltage.

The thickness of the electron transport layer may be in a range of about 100 Å to about 1,000 Å, and in some embodiments, about 150 Å to about 500 Å. When the thickness of the electron transport layer is within any of these ranges, excellent electron transport characteristics may be obtained without a substantial increase in driving voltage.

The electron transport region 17 (e.g., the electron transport layer in the electron transport region 17) may further include, in addition to the materials described above, a material including metal.

The metal-containing material may include at least one an alkali metal complex, an alkaline earth metal complex, or any combination thereof. The alkali metal complex may include a metal ion of a lithium (Li) ion, a sodium (Na) ion, a potassium (K) ion, a rubidium (Rb) ion, a cesium (Cs) ion, or any combination thereof. The alkaline earth metal complex may include a metal ion a beryllium (Be) ion, a magnesium (Mg) ion, a calcium (Ca) ion, a strontium (Sr) ion, a barium (Ba) ion, or any combination thereof. Each ligand coordinated with the metal ion of the alkali metal complex and the alkaline earth metal complex may independently be hydroxyquinoline, hydroxyisoquinoline, hydroxybenzoquinoline, hydroxyacridine, hydroxyphenanthridine, hydroxyphenyloxazole, hydroxyphenylthiazole, hydroxyphenyloxadiazole, hydroxyphenylthiadiazole, hydroxyphenylpyridine, hydroxyphenylbenzimidazole, hydroxyphenylbenzothiazole, bipyridine, phenanthroline, or cyclopentadiene, but embodiments are not limited thereto.

For example, the metal-containing material may include a Li complex. The Li complex may include, e.g., Compound ET-D1 (LiQ) or Compound ET-D2:

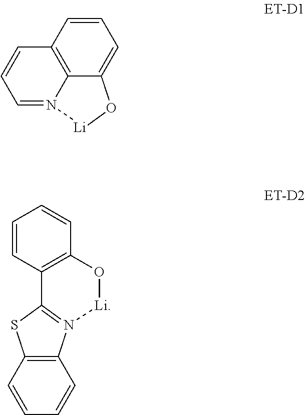

The electron transport region 17 may include an electron injection layer that facilitates injection of electrons from the second electrode 19. The electron injection layer may be in direct contact with the second electrode 19.

The electron injection layer may have i) a single-layered structure consisting of a single layer consisting of a single material, ii) a single-layered structure consisting of a single layer including a plurality of different materials, or iii) a multi-layered structure having a plurality of layers, each including a plurality of different materials.

The electron injection layer may include an alkali metal, an alkaline earth metal, a rare earth metal, an alkali metal compound, an alkaline earth metal compound, a rare earth metal compound, an alkali metal complex, an alkaline earth metal complex, a rare earth metal complex, or a combination thereof.

The alkali metal may be Li, Na, K, Rb, or Cs. In some embodiments, the alkali metal may be Li, Na, or Cs. In one or more embodiments, the alkali metal may be L₁ or Cs, but embodiments are not limited thereto.

The alkaline earth metal may be Mg, Ca, Sr, or Ba.

The rare earth metal may be Sc, Y, Ce, Tb, Yb, or Gd.

The alkali metal compound, the alkaline earth metal compound, and the rare earth metal compound may each independently be an oxide and a halides (e.g., fluorides, chlorides, bromides, or iodides) of the alkali metal, the alkaline earth metal, and the rare earth metal, respectively.

The alkali metal compound may be alkali metal oxides, such as Li₂O, Cs₂O, or K₂O, and alkali metal halides, such as LiF, NaF, CsF, KF, Lil, Nal, Csl, or KI. In some embodiments, the alkali metal compound may be LiF, Li₂O, NaF, Lil, Nal, Csl, or KI, but embodiments are not limited thereto.

The alkaline earth-metal compound may be an alkaline earth-metal compound, such as BaO, SrO, CaO, BaxSr₁₋ₓO (wherein 0<x<1), or BaxCa₁₋ₓO (wherein 0<x<1). In some embodiments, the alkaline earth metal compound may be BaO, SrO, or CaO, but embodiments are not limited thereto.

The rare earth metal compound may be YbF₃, ScF₃, ScO₃, Y₂O₃, Ce₂O₃, GdF₃, or TbF₃. In some embodiments, the rare earth metal compound may be YbF₃, ScF₃, TbF₃, Ybl₃, Scl₃, or Tbl₃, but embodiments are not limited thereto.

The alkali metal complex, the alkaline earth metal complex, and the rare earth metal complex may each include ions of the above-described alkali metal, alkaline earth metal, and rare earth metal. Each ligand coordinated with the metal ion of the alkali metal complex, the alkaline earth metal complex, and the rare earth metal complex may independently be hydroxyquinoline, hydroxyisoquinoline, hydroxybenzoquinoline, hydroxyacridine, hydroxyphenanthridine, hydroxyphenyloxazole, hydroxyphenylthiazole, hydroxyphenyloxadiazole, hydroxyphenylthiadiazole, hydroxyphenylpyridine, hydroxyphenylbenzimidazole, hydroxyphenylbenzothiazole, bipyridine, phenanthroline, or cyclopentadiene, but embodiments are not limited thereto.

The electron injection layer may consist of an alkali metal, an alkaline earth metal, a rare earth metal, an alkali metal compound, an alkaline earth metal compound, a rare earth metal compound, an alkali metal complex, an alkaline earth metal complex, a rare earth metal complex, or a combination thereof, as described above. In some embodiments, the electron injection layer may further include an organic material. When the electron injection layer further includes an organic material, the alkali metal, the alkaline earth metal, the rare earth metal, the alkali metal compound, the alkaline earth metal compound, the rare earth metal compound, the alkali metal complex, the alkaline earth metal complex, the rare earth metal complex, or a combination thereof, the foregoing may be homogeneously or non-homogeneously dispersed in a matrix including the organic material.

The thickness of the electron injection layer may be in a range of about 1 Å to about 100 Å, and in some embodiments, about 3 Å to about 90 Å. When the thickness of the electron injection layer is within any of these ranges, excellent electron injection characteristics may be obtained without a substantial increase in driving voltage. Second electrode 19

The second electrode 19 may be on the organic layer 10A. In an embodiment, the second electrode 19 may be a cathode that is an electron injection electrode. In this embodiment, a material for forming the second electrode 19 may be a material having a low work function, for example, a metal, an alloy, an electrically conductive compound, or a combination thereof.

The second electrode 19 may include at least one lithium (Li), silver (Ag), magnesium (Mg), aluminum (Al), aluminum-lithium (Al—Li), calcium (Ca), magnesium-indium (Mg—In), magnesium-silver (Mg—Ag), ITO, IZO, or any combination thereof, but embodiments are not limited thereto. The second electrode 19 may be a transmissive electrode, a semi-transmissive electrode, or a reflective electrode.

The second electrode 19 may have a single-layered structure or a multi-layered structure including two or more layers.

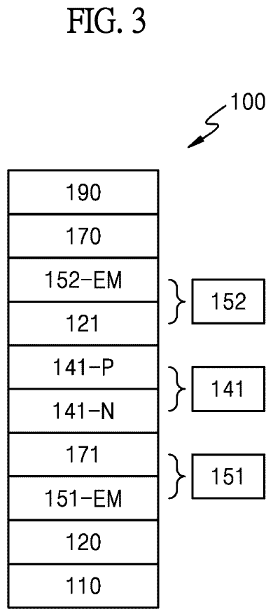

Hereinbefore the organic light-emitting device 10 has been described with reference to FIG. 1, but embodiments are not limited thereto. Description of FIG. 3

FIG. 3 is a schematic view of an organic light-emitting device 100 according to an exemplary embodiment.

The organic light-emitting device 100 in FIG. 3 may include a first electrode 110, a second electrode 190 facing the first electrode 110, and a first light-emitting unit 151 and a second light-emitting unit 152 between the first electrode 100 and the second electrode 190. A charge generating layer 141 may be between the first light-emitting unit 151 and the second light-emitting unit 152, and the charge generating layer 141 may include an n-type charge generating layer 141-N and a p-type charge generating layer 141-P. The charge generating layer 141 is a layer serving to generate charges and supply the generated charges to the adjacent light-emitting unit, and may include a known material.

The first light-emitting unit 151 may include a first emission layer 151-EM, and the second light-emitting unit 152 may include a second emission layer 152-EM. A maximum emission wavelength of light emitted by the first light-emitting unit 151 may be different from a maximum emission wavelength of light emitted by the second light-emitting unit 152. For example, mixed light of the light emitted by the first light-emitting unit 151 and the light emitted by the second light-emitting unit 152 may be white light, but embodiments are not limited thereto.

A hole transport region 120 may be between the first light-emitting unit 151 and the first electrode 110, and the second light-emitting unit 152 may include a first hole transport region 121 toward the first electrode 110.

An electron transport region 170 may be between the second light-emitting unit 152 and the second electrode 190, and the first light-emitting unit 151 may include a first electron transport region 171 between the charge generating layer 141 and the first emission layer 151-EM.

In some embodiments, the first emission layer 151-EM may include the heterocyclic compound.

In some embodiments, the second emission layer 152-EM may include the heterocyclic compound.

In FIG. 3, the first electrode 110 and the second electrode 190 may each be understood by referring to the descriptions for the first electrode 110 and the second electrode 19 in FIG. 1, respectively.

In FIG. 3, the first emission layer 151-EM and the second emission layer 152-EM may each be understood by referring to the descriptions for the emission layer 15 in FIG. 3.

In FIG. 3, the hole transport region 120 and the first hole transport region 121 may each be understood by referring to the descriptions for the hole transport region 12 in FIG. 1.

In FIG. 3, the electron transport region 170 and the first electron transport region 171 may each be understood by referring to the descriptions for the electron transport region 17 in FIG. 1.

Hereinbefore, by referring to FIG. 3, the first light-emitting unit 151 and the second light-emitting unit 152 has been described as being included in an organic light-emitting device including an emission layer including the host, the dopant, and the sensitizer described herein. However, the organic light-emitting device 100 in FIG. 3 may be subjected to various modifications, for example, at least one of the first light-emitting unit 151 and the second light-emitting unit 152 of the organic light-emitting device 100 in FIG. 3 may be replaced by any suitable known light-emitting unit, or three or more light-emitting units may be included.

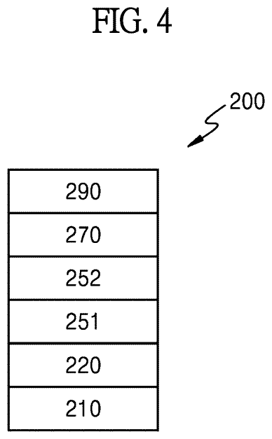

### Description of FIG. 4

FIG. 4 is a schematic view of an organic light-emitting device 200 according to an exemplary embodiment.

The organic light-emitting device 100 in FIG. 4 includes a first electrode 210, a second electrode 290 facing the first electrode 210, and a first emission layer 251 and a second emission layer 252 between the first electrode 210 and the second electrode 290.

A maximum emission wavelength of light emitted by the first emission layer 251 may be different from a maximum emission wavelength of light emitted by the second emission layer 252. For example, mixed light of the light emitted by the first emission layer 251 and the light emitted by the second emission layer 252 may be white light, but embodiments are not limited thereto.

A hole transport region 220 may be between the first emission layer 251 and the first electrode 210, and an electron transport region 270 may be between the second emission layer 252 and the second electrode 290.

In some embodiments, the first emission layer 251 may include the heterocyclic compound.

In some embodiments, the second emission layer 252 may include the heterocyclic compound.

In FIG. 4, the first electrode 210, the hole transport region 220, and the second electrode 290 may each be understood by referring to the descriptions for the first electrode 11, the hole transport region 12, and the second electrode 19 in FIG. 1, respectively.

In FIG. 4, the first emission layer 251 and the second emission layer 252 may each be understood by referring to the descriptions for the emission layer 15 in FIG. 1.

In FIG. 4, the electron transport region 170 may be understood by referring to the descriptions for the electron transport region 17 in FIG. 1.

Hereinbefore, by referring to FIG. 4, the first emission layer 251 and the second emission layer 252 has been described as being included in an organic light-emitting device including the heterocyclic compound described herein. However, the organic light-emitting device in FIG. 4 may be subjected to various modifications, for example, one of the first emission layer 251 and the second emission layer 252 may be replaced by any suitable known layer, three or more layers may be included, or an interlayer may be further located between neighboring emission layers. Electronic apparatus

The organic light-emitting device may be included in various electronic apparatuses.

The electronic apparatus may further include a thin-film transistor, in addition to the organic light-emitting device. The thin-film transistor may include a source electrode, a drain electrode, and an activation layer, wherein one of the source electrode and the drain electrode may be electrically connected to one of the first electrode and the second electrode of the organic light-emitting device.

### General Definitions of Terms

The term “C₁-C₆₀ alkyl group” as used herein refers to a linear or branched saturated aliphatic hydrocarbon monovalent group having 1 to 60 carbon atoms. Examples thereof include a methyl group, an ethyl group, a propyl group, an iso-butyl group, a sec-butyl group, a tert-butyl group, a pentyl group, an iso-amyl group, and a hexyl group. The term “C₁-C₆₀ alkylene group” as used herein refers to a divalent group having the same structure as the C₁-C₆₀ alkyl group.

The term “C₁-C₆₀ alkoxy group” as used herein refers to a monovalent group represented by —OA₁₀₁ (wherein A₁₀₁ is a C₁-C₁₀ alkyl group). Examples thereof include a methoxy group, an ethoxy group, and an isopropyloxy group.

The term “C₂-C₆₀ alkenyl group” as used herein refers to a group formed by placing at least one carbon-carbon double bond in the middle or at the terminus of the C₂-C₆₀ alkyl group. Examples thereof include an ethenyl group, a propenyl group, and a butenyl group. The term “C₂-C₆₀ alkenylene group” as used herein refers to a divalent group having the same structure as the C₂-C₆₀ alkenyl group.

The term “C₂-C₆₀ alkynyl group” as used herein refers to a group formed by placing at least one carbon-carbon triple bond in the middle or at the terminus of the C₂-C₆₀ alkyl group. Examples thereof include an ethenyl group and a propenyl group. The term “C₂-C₆₀ alkynylene group” as used herein refers to a divalent group having the same structure as the C₂-C₆₀ alkynyl group.

The term “C₃-C₁₀ cycloalkyl group” as used herein refers to a monovalent monocyclic saturated hydrocarbon group including 3 to 10 carbon atoms. Examples thereof include a cyclopropyl group, a cyclobutyl group, a cyclopentyl group, a cyclohexyl group, and a cycloheptyl group. The term “C₃-C₁₀ cycloalkylene group” as used herein refers to a divalent group having the same structure as the C₃-C₁₀ cycloalkyl group.

The term “C₁-C₁₀ heterocycloalkyl group” as used herein refers to a monovalent monocyclic group including at least one heteroatom of N, O, P, Si, S, B, Se, Ge, Te, or any combination thereof as a ring-forming atom and 1 to 10 carbon atoms. Examples thereof include a tetrahydrofuranyl group and a tetrahydrothiophenyl group. The term “C₁-C₁₀ heterocycloalkylene group” as used herein refers to a divalent group having the same structure as the C₁-C₁₀ heterocycloalkyl group.

The term “C₃-C₁₀ cycloalkenyl group” as used herein refers to a monovalent monocyclic group that has 3 to 10 carbon atoms and at least one carbon-carbon double bond in its ring, wherein the molecular structure as a whole is non-aromatic. Examples thereof include a cyclopentenyl group, a cyclohexenyl group, and a cycloheptenyl group. The term “C₃-C₁ cycloalkenylene group” as used herein refers to a divalent group having the same structure as the C₃-C₁ cycloalkenyl group.

The term “C₁-C₁₀ heterocycloalkenyl group” as used herein refers to a monovalent monocyclic group including at least one heteroatom of N, O, P, Si, S, B, Se, Ge, Te, or any combination thereof as a ring-forming atom, 1 to 10 carbon atoms, and at least one double bond in its ring. Examples of the C₁-C₁₀ heterocycloalkenyl group include a 2,3-dihydrofuranyl group and a 2,3-dihydrothiophenyl group. The term “C₁-C₁₀ heterocycloalkylene group” as used herein refers to a divalent group having the same structure as the C₁-C₁₀ heterocycloalkyl group.

The term “C₆-C₆₀ aryl group” as used herein refers to a monovalent group having a carbocyclic aromatic system having 6 to 60 carbon atoms. The term “C₆-C₆₀ arylene group” as used herein refers to a divalent group having a carbocyclic aromatic system having 6 to 60 carbon atoms. Examples of the C₆-C₆₀ aryl group include a phenyl group, a naphthyl group, an anthracenyl group, a phenanthrenyl group, a pyrenyl group, and a chrysenyl group. When the C₆-C₆₀ aryl group and the C₆-C₆₀ arylene group each include a plurality of rings, the plurality of rings may be fused to each other.

The term “C₁-C₆₀ heteroaryl group” as used herein refers to a monovalent group having a heterocyclic aromatic system having at least one heteroatom N, O, P, Si, S, B, Se, Ge, Te, or any combination thereof as a ring-forming atom and 1 to 60 carbon atoms. The term “C₁-C₆₀ heteroarylene group” as used herein refers to a divalent group having a heterocyclic aromatic system having at least one heteroatom N, O, P, Si, S, B, Se, Ge, Te, or any combination thereof as a ring-forming atom and 1 to 60 carbon atoms. Examples of the C₁-C₆₀ heteroaryl group include a pyridinyl group, a pyrimidinyl group, a pyrazinyl group, a pyridazinyl group, a triazinyl group, a quinolinyl group, and an isoquinolinyl group. When the C₁-C₆₀ heteroaryl group and the C₁-C₆₀ heteroarylene group each include a plurality of rings, the plurality of rings may be fused to each other.

The term “C₆-C₆₀ aryloxy group” as used herein is represented by —OA₁₀₂ (wherein A₁₀₂ is the C₆-C₆₀ aryl group). The term “C₆-C₆₀ arylthio group” as used herein is represented by —SA₁₀₃ (wherein A₁₀₃ is the C₆-C₆₀ aryl group).

The term “monovalent aromatic condensed polycyclic group” as used herein refers to a monovalent group having two or more rings condensed and only carbon atoms (for example, the number of carbon atoms may be in a range of 8 to 60) as ring-forming atoms, wherein the molecular structure as a whole is aromatic. The term “divalent aromatic condensed polycyclic group” as used herein refers to a divalent group having substantially the same structure as the monovalent aromatic condensed polycyclic group.

The term “monovalent aromatic condensed heteropolycyclic group” as used herein refers to a monovalent group having at least two rings condensed and a heteroatom N, O, P, Si, S, B, Se, Ge, Te, or any combination thereof as well as carbon atoms (for example, the number of carbon atoms may be in a range of 1 to 60) as ring-forming atoms, wherein the molecular structure as a whole is aromatic. The term “divalent aromatic condensed heteropolycyclic group” as used herein refers to a divalent group having substantially the same structure as the monovalent aromatic condensed heteropolycyclic group.

The term “monovalent non-aromatic condensed polycyclic group” as used herein refers to a monovalent group having two or more rings condensed and only carbon atoms (for example, the number of carbon atoms may be in a range of 8 to 60) as ring-forming atoms, wherein the molecular structure as a whole is non-aromatic. Examples of the non-aromatic condensed polycyclic group include a fluorenyl group. The term “divalent non-aromatic condensed polycyclic group” as used herein refers to a divalent group having substantially the same structure as the monovalent non-aromatic condensed polycyclic group.

The term “monovalent non-aromatic condensed heteropolycyclic group” as used herein refers to a monovalent group having at least two rings condensed and a heteroatom N, O, P, Si, S, B, Se, Ge, Te, or any combination thereof as well as carbon atoms (for example, the number of carbon atoms may be in a range of 1 to 60) as ring-forming atoms, wherein the molecular structure as a whole is non-aromatic. Examples of the monovalent non-aromatic condensed heteropolycyclic group include a carbazolyl group. The term “divalent non-aromatic condensed heteropolycyclic group” as used herein refers to a divalent group having substantially the same structure as the monovalent non-aromatic condensed heteropolycyclic group.

The term “C₅-C₃₀ carbocyclic group” as used herein refers to a saturated or unsaturated cyclic group including 5 to 30 carbon atoms only as ring-forming atoms. The C₅-C₃₀ carbocyclic group may be a monocyclic group or a polycyclic group. Depending on formula structure, the C₅-C₃₀ carbocyclic group may be monovalent, divalent, trivalent, quadrivalent, pentavalent, or hexavalent.

The term “C₁-C₃₀ heterocyclic group” as used herein refers to saturated or unsaturated cyclic group including 1 to 30 carbon atoms and at least one heteroatom N, O, P, Si, S, B, Se, Ge, Te, or any combination thereof as ring-forming atoms. The C₁-C₃₀ heterocyclic group may be a monocyclic group or a polycyclic group. Depending on formula structure, the C₁-C₃₀ heterocyclic group may be monovalent, divalent, trivalent, quadrivalent, pentavalent, or hexavalent.

A substituent of the substituted C₅-C₃₀ carbocyclic group, the substituted C₁-C₃₀ heterocyclic group, the substituted C₁-C₆₀ alkyl group, the substituted C₂-C₆₀ alkenyl group, the substituted C₂-C₆₀ alkynyl group, the substituted C₁-C₆₀ alkoxy group, the substituted C₃-C₁₀ cycloalkyl group, the substituted C₁-C₁₀ heterocycloalkyl group, the substituted C₃-C₁₀ cycloalkenyl group, the substituted C₁-C₁₀ heterocycloalkenyl group, the substituted C—C₆₀ aryl group, the substituted C—C₆₀ aryloxy group, the substituted C—C₆₀ arylthio group, the substituted C₁-C₆₀ heteroaryl group, the substituted monovalent aromatic condensed polycyclic group, the substituted monovalent aromatic condensed heteropolycyclic group, the substituted monovalent non-aromatic condensed polycyclic group, and the substituted monovalent non-aromatic condensed heteropolycyclic group may be:

deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, or a C₁-C₆₀ alkoxy group;

a C₁-C₆₀ alkyl group, a C₂-C₆₀ alkenyl group, a C₂-C₆₀ alkynyl group, or a C₁-C₆₀ alkoxy group, each substituted with at least one deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₃-C₁ cycloalkyl group, a C₁-C₁₀ heterocycloalkyl group, a C₃-C₁ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, monovalent aromatic condensed polycyclic group, monovalent aromatic condensed heteropolycyclic group, a monovalent non-aromatic condensed polycyclic group, a monovalent non-aromatic condensed heteropolycyclic group, —N(Q₁₁)(Q₁₂), —Si(Q₁₃)(Q₁₄)(Q₁₅), —B(Q₁₆)(Q₁₇), —P(═O)(Q₁₈)(Q₁₉), or any combination thereof;

a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent aromatic condensed polycyclic group, a monovalent aromatic condensed heteropolycyclic group, a monovalent non-aromatic condensed polycyclic group, or a monovalent non-aromatic condensed heteropolycyclic group;

a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent aromatic condensed polycyclic group, a monovalent aromatic condensed heteropolycyclic group, a monovalent non-aromatic condensed polycyclic group, or a monovalent non-aromatic condensed heteropolycyclic group, each substituted with at least one deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent aromatic condensed polycyclic group, a monovalent aromatic condensed heteropolycyclic group, a monovalent non-aromatic condensed polycyclic group, a monovalent non-aromatic condensed heteropolycyclic group, —N(Q₂₁)(Q₂₂), —Si(Q₂₃)(Q₂₄)(Q₂₅), —B(Q₂₆)(Q₂₇), —P(═O)(Q₂₈)(Q₂₉), or any combination thereof; or

—N(Q₃₁)(Q₃₂), —Si(Q₃₃)(Q₃₄)(Q₃₅), —B(Q₃₆)(Q₃₇), or —P(═O)(Q₃₈)(Q₃₉),

wherein Q₁ to Q₉, Q₁₁ to Q₁₉, Q₂₁ to Q₂₉, and Q₃₁ to Q₃₉ may each independently be a C₆-C₆₀ aryl group, a C₆-C₆₀ aryloxy group, a C₆-C₆₀ arylthio group, a C₁-C₆₀ heteroaryl group, a monovalent aromatic condensed polycyclic group, a monovalent aromatic condensed heteropolycyclic group, a monovalent non-aromatic condensed polycyclic group, or a monovalent non-aromatic condensed heteropolycyclic group, each substituted with at least one of hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₁-C₆₀ alkyl group, a C₆-C₆₀ aryl group, or any combination thereof.

The term “room temperature” as used herein refers to a temperature of about 25° C.

As used herein, the number of carbons in each group that is substituted (e.g., C₁-C₆₀) excludes the number of carbons in the substituent. For example, a C₁-C₆₀ alkyl group can be substituted with a C₁-C₆₀ alkyl group. The total number of carbons included in the C₁-C₆₀ alkyl group substituted with the C₁-C₆₀ alkyl group is not limited to 60 carbons. In addition, more than one C₁-C₆₀ alkyl substituent may be present on the C₁-C₆₀ alkyl group. This definition is not limited to the C₁-C₆₀ alkyl group and applies to all substituted groups that recite a carbon range.

The terms “a biphenyl group, a terphenyl group, and a tetraphenyl group” as used herein each refer to a monovalent group having two, three, and four phenyl groups linked via a single bond, respectively.

The terms “a cyano group-containing phenyl group, a cyano group-containing biphenyl group, a cyano group-containing terphenyl group, and a cyano group-containing tetraphenyl group” as used herein each refer to a phenyl group, a biphenyl group, a terphenyl group, and a tetraphenyl group, each substituted with at least one cyano group. In “the cyano group-containing phenyl group, the cyano group-containing biphenyl group, the cyano group-containing terphenyl group, and the cyano group-containing tetraphenyl group”, a cyano group may be substituted at any position, and “the cyano group-containing phenyl group, the cyano group-containing biphenyl group, the cyano group-containing terphenyl group, and the cyano group-containing tetraphenyl group” may further include a substituent in addition to a cyano group. For example, “a phenyl group substituted with a cyano group‘ and’ a phenyl group substituted with a methyl group” all belong to “a cyano group-containing phenyl group”.

Hereinafter, an organic light-emitting device, according to an embodiment, will be described in more detail with reference to Synthesis Examples and Examples; however, the present disclosure is not limited thereto. The wording “B was used instead of A” used in describing Synthesis Examples means that an identical molar equivalent of B was used in place of A.

### EXAMPLES

The following compounds are synthesized in Synthesis Examples 1 to 21:

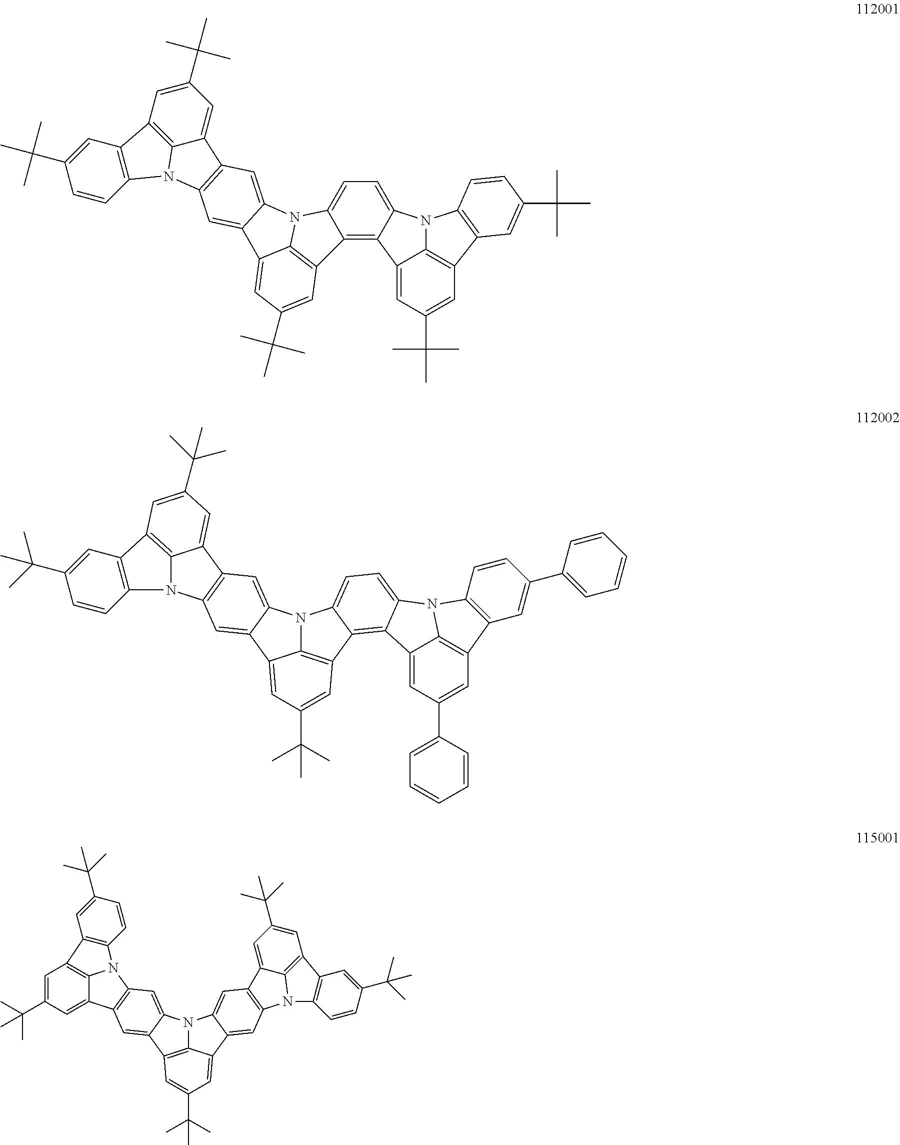

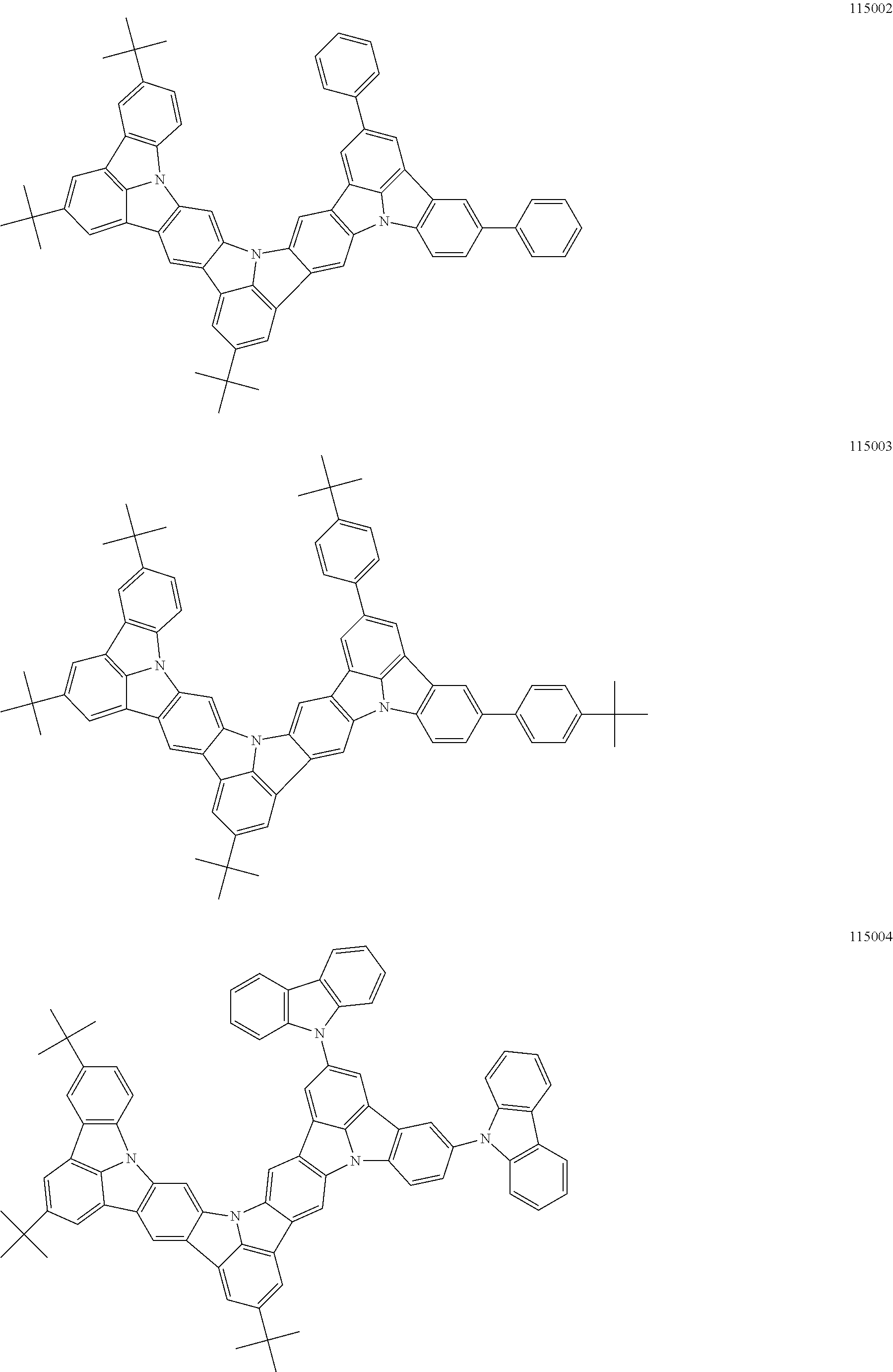

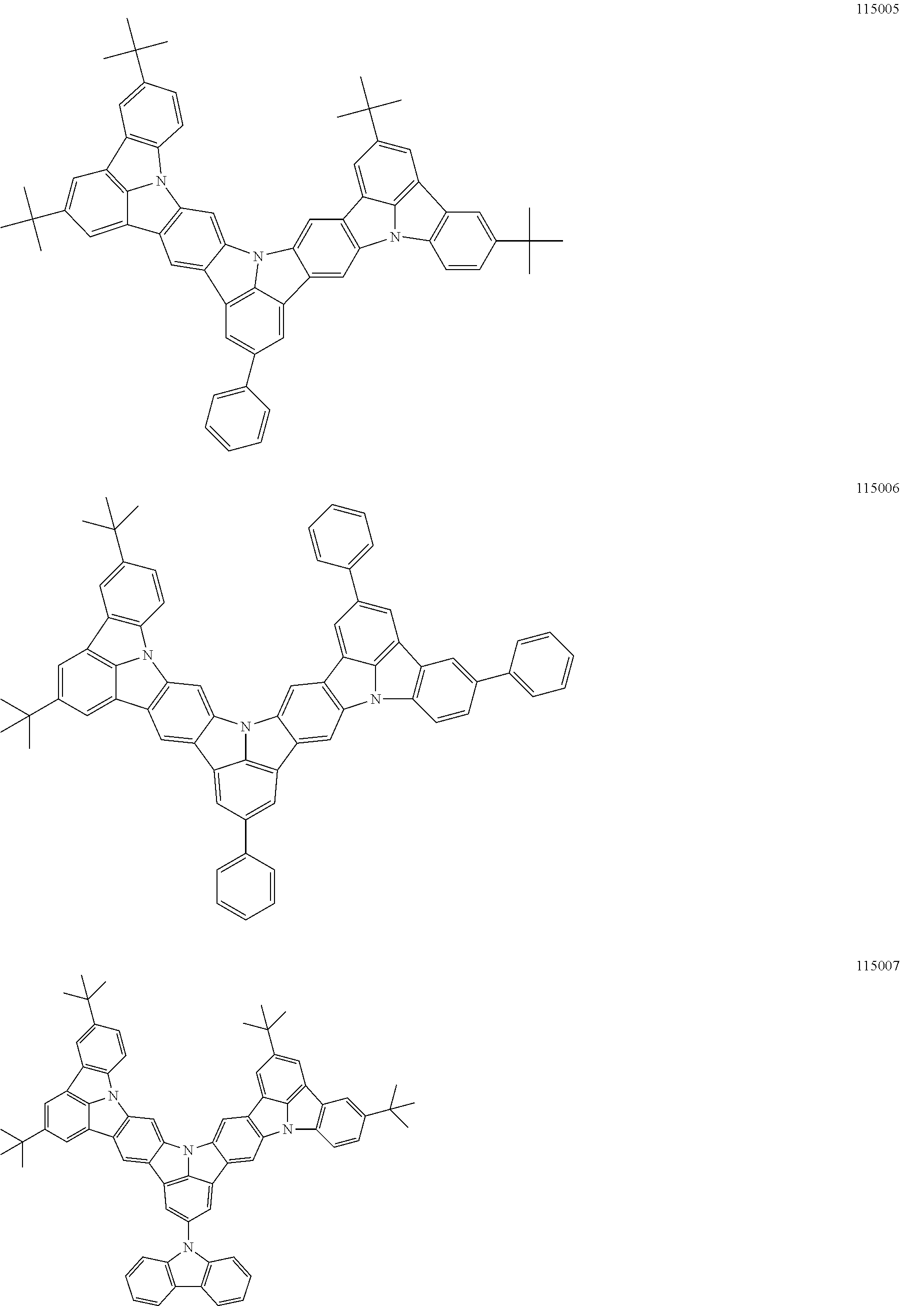

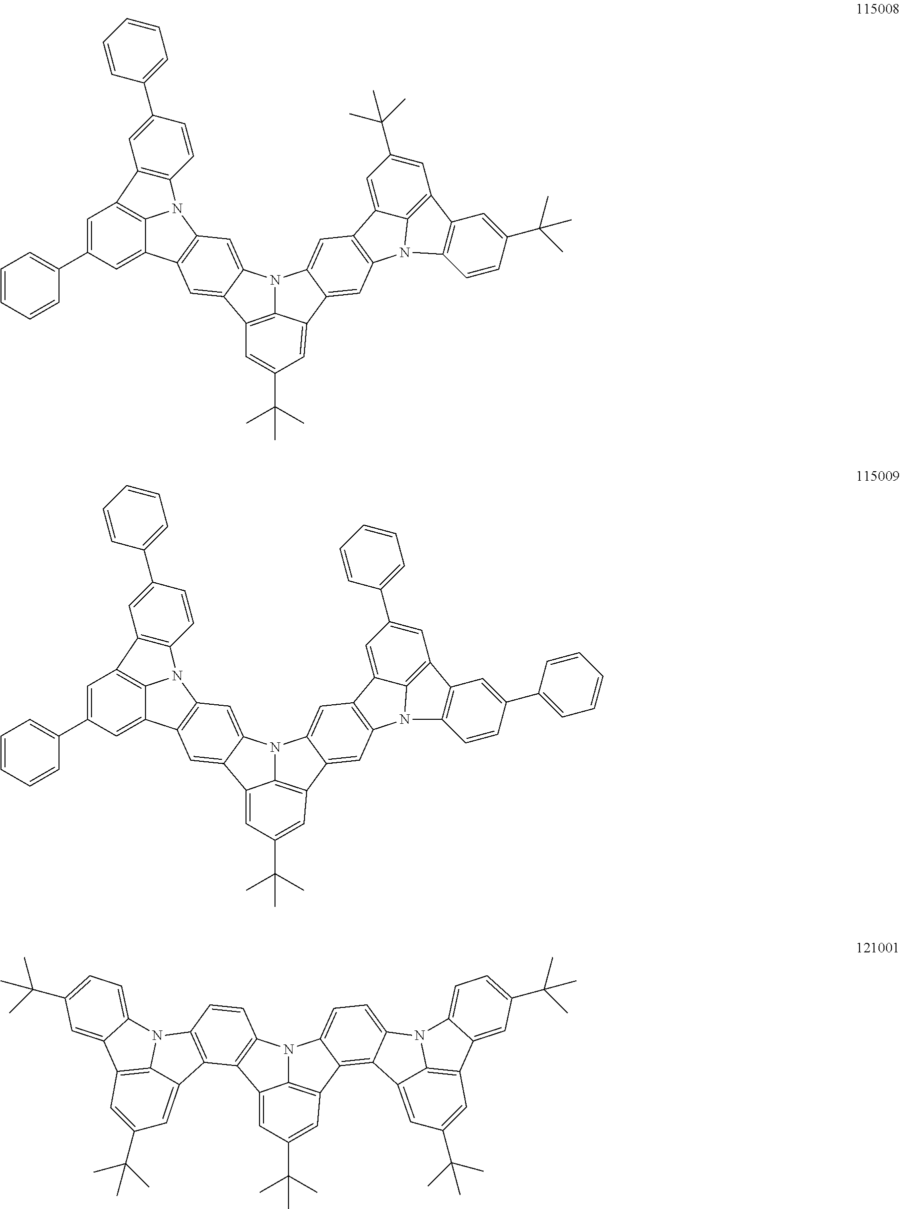

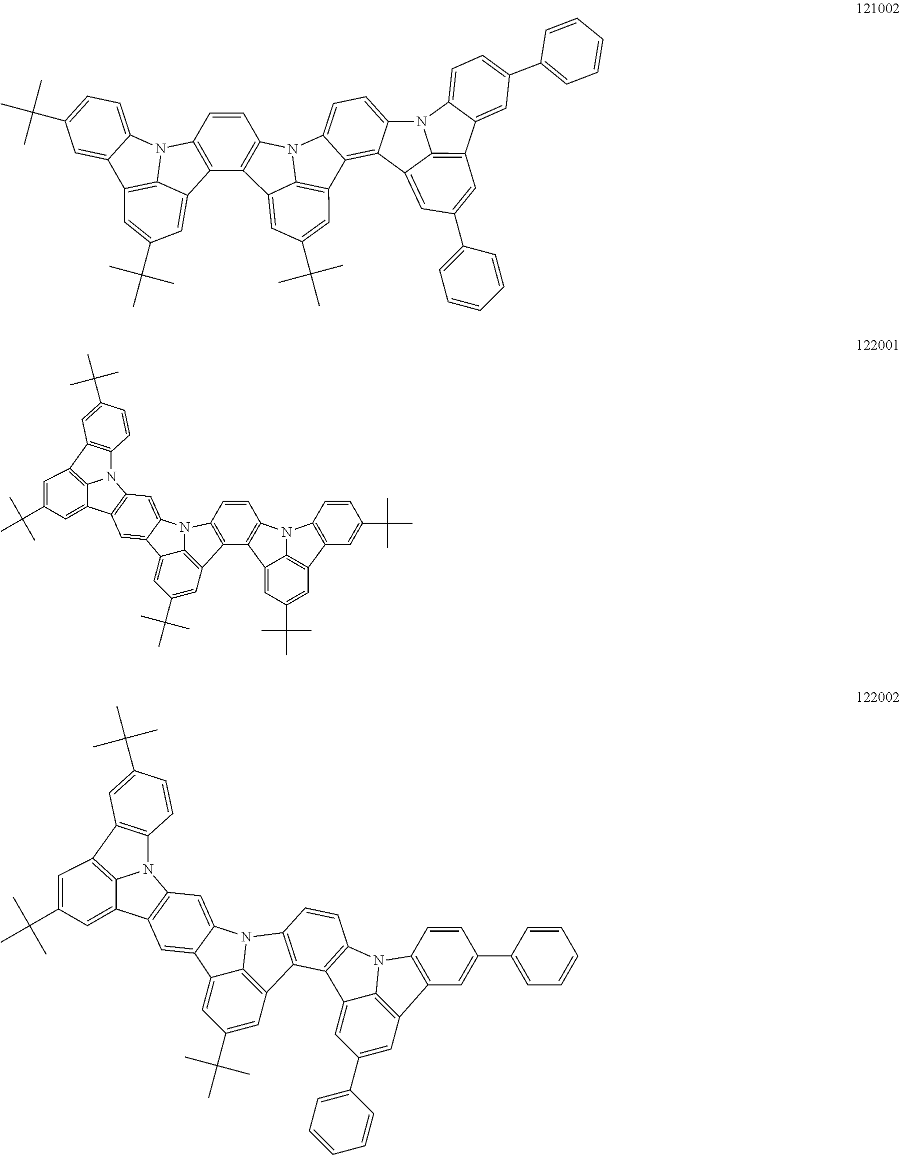

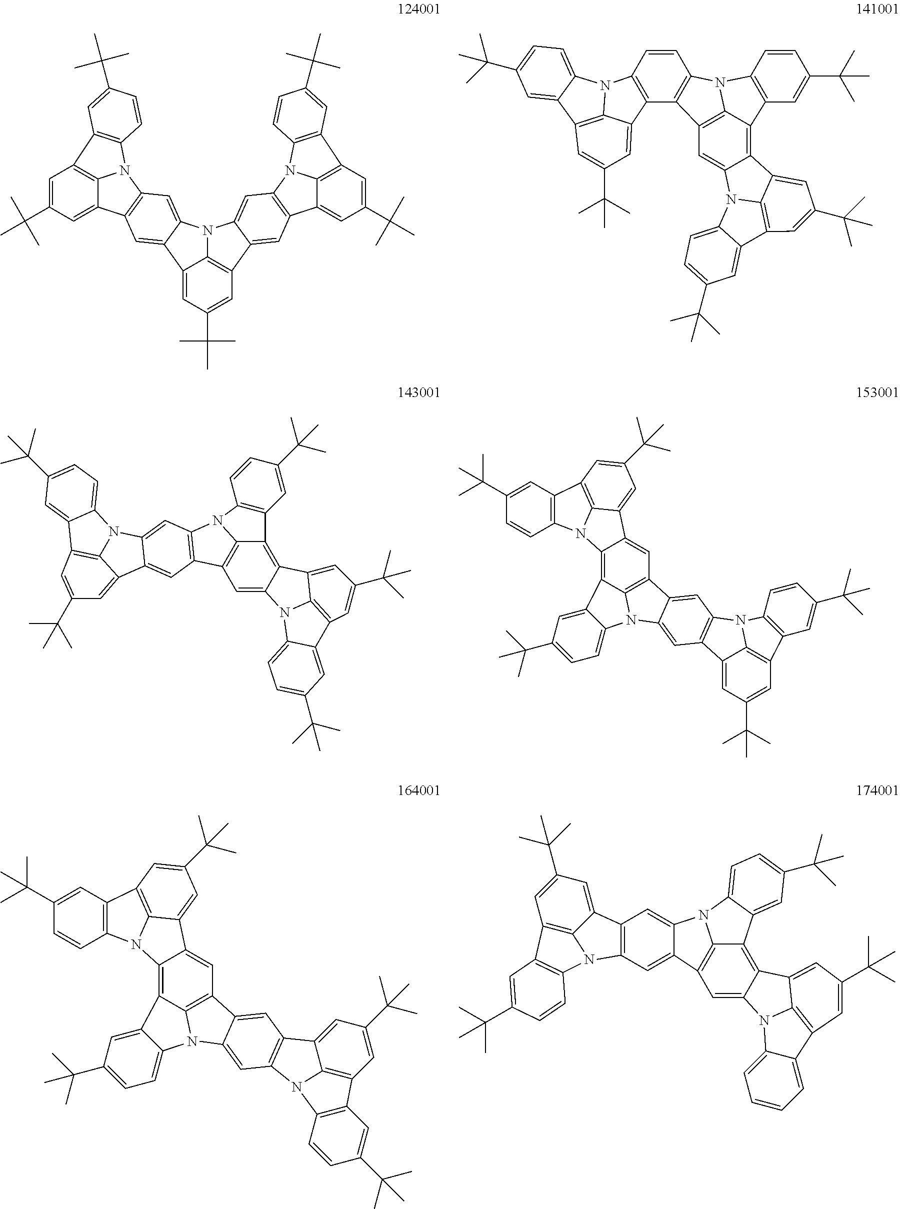

### Synthesis Example 1: Synthesis of Compound 112001

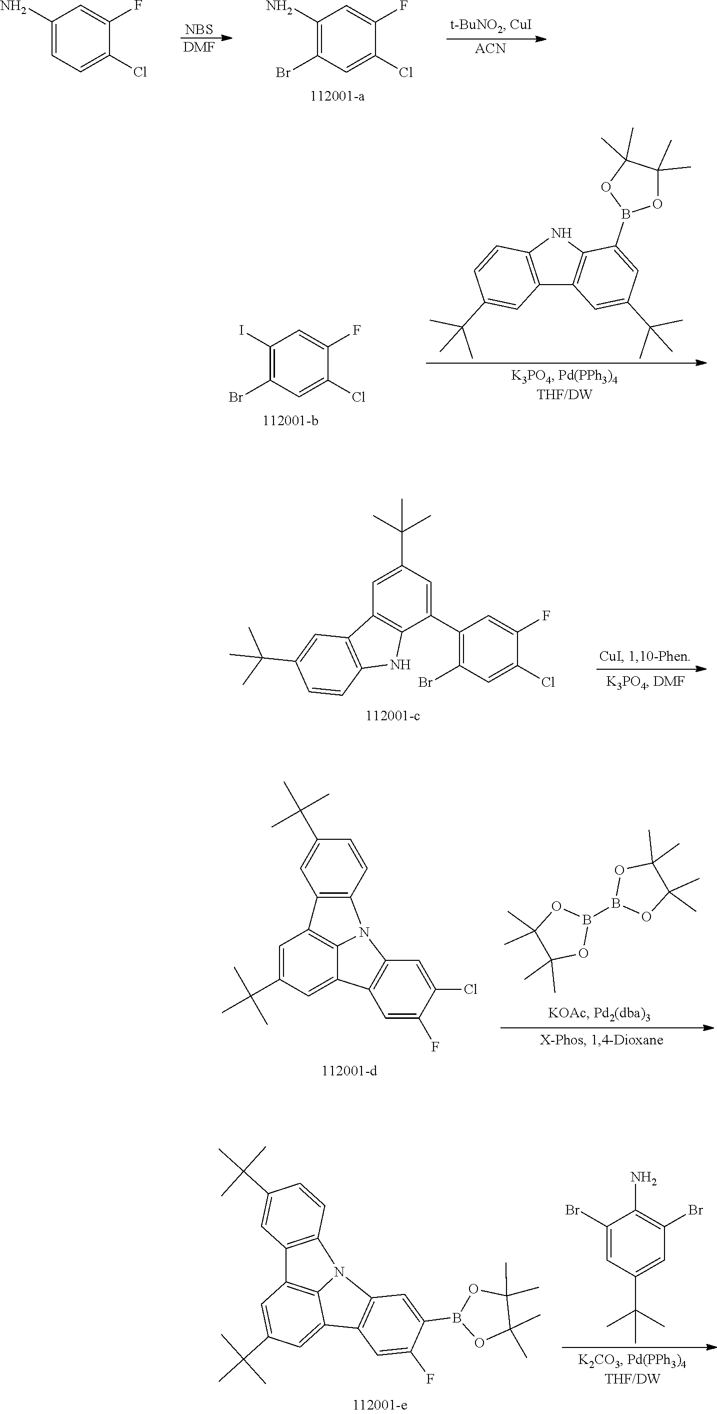

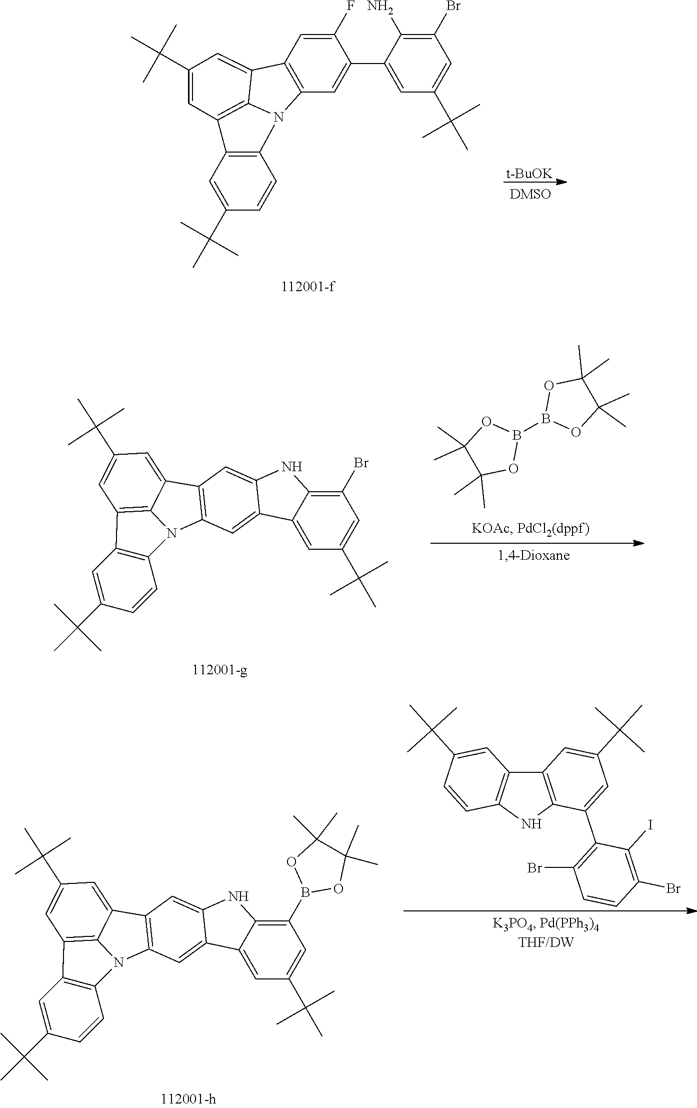

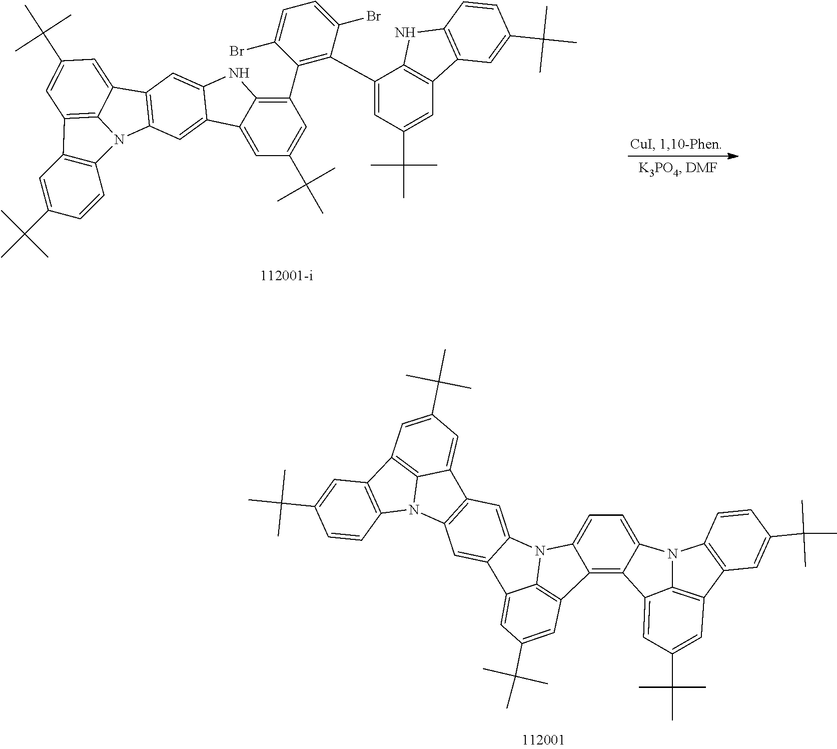

### Synthesis of Intermediate 112001-a

20.0 grams (g) (137 millimoles (mmol)) of 4-chloro-3-fluoroaniline, 25.7 g (144 mmol) of N-bromosuccinimide, and N,N-dimethylformamide (DMF) were stirred at room temperature to allow a reaction to occur. Once the reaction was complete, a sodium thiosulfate aqueous solution (2M) was added dropwise thereto. Distilled water and dichloromethane (DCM) were added thereto, followed by extraction. Then, an aqueous solution layer was removed therefrom. The resulting filtrate was concentrated under reduced pressure. The product was separated through column chromatography to obtain 29.3 g of Intermediate 112001 (yield: 95%).

LC-Mass (calculated value: 223.93 g/mol, found value: 223.16 (M+1)) Synthesis of Intermediate 112001-b

36.0 g (160 mmol) of Intermediate 112001-a, 45.7 g (240 mmol) of copper(I) iodide (CuI), 19.8 g (192 mmol) of tert-butyl nitrite, and 800 mL of acetonitrile were stirred under reflux at a temperature of 150° C. Once the reaction was complete, the resulting mixture was cooled to room temperature and filtrated through silica gel under reduced pressure to obtain a filtrate. The resulting filtrate was concentrated under reduced pressure and separated through silica gel column chromatography to thereby obtain 21.3 g of Intermediate 112001-b (yield: 40%).

LC-Mass (calculated value: 334.81 g/mol, found value: 334.25(M+1))

### Synthesis of Intermediate 112001-c

37.2 g (111 mmol) of Intermediate 112001-b, 15.0 g (37.0 mmol) of 3,6-di-tert-butyl-1-(4,4,5,5-tetramethyl-1,3,2-dioxaborolan-2-yl)-9H-carbazole, and 1.28 g (1.11 mmol) of tetrakis(triphenylphosphine)palladium(0), Pd(PPh₃)₄) were added to 300 mL of tetrahydrofuran (THF), followed by stirring under reflux. The mixture was added dropwise to 150 mL of 2 M potassium triphosphate (K₃PO₄) solution and stirred. Once the reaction was complete, an organic layer was separated using dichloromethane and distilled water. Then, the separated organic layer was concentrated under reduced pressure and separated through silica gel column chromatography to thereby 8.72 g of Intermediate 112001-c (yield: 48%).

LC-Mass (calculated value: 486.10 g/mol, found value: 486.75 (M+1))

### Synthesis of Intermediate 112001-d

8.70 g (17.9 mmol) of Intermediate 112001-c, 1.70 g (8.95 mmol) of Cul, 1.61 g (8.95 mmol) of 1,10-phenanthroline, 11.4 g (54.7 mmol) of potassium phosphate, and 100 ml of DMF were heated at a temperature of 100° C. and stirred. Once the reaction was complete, the resulting mixture was filtrated under reduced pressure using silica gel. Then, the filtrate was concentrated under reduced pressure. Subsequently, a precipitate obtained by using dichloromethane and methanol was filtered, and the resulting solid was dried in a vacuum oven to thereby obtain 6.53 g of Intermediate 112001-d (yield: 90%).

LC-Mass (calculated value: 405.17 g/mol, found value: 405.59 (M+1))

### Synthesis of Intermediate 112001-e

6.50 g (16.0 mmol) of Intermediate 112001-d, 6.10 g (24.1 mmol) of 4,4,4′,4′,5,5,5′,5′-octamethyl-2,2′-bi(1,3,2-dioxaborolane), 4.71 g (47.8 mmol) of potassium acetate (KOAc), 0.733 g (0.801 mmol) of tris(dibenzylideneacetone)dipalladium(0) (Pd₂(dba)₃), 0.764 g (1.60 mmol) of X-Phos, and 120 mL of 1,4-dioxane were added to a round flask. Then, a reaction was allowed to occur while heating at a temperature of 170° C. and stirring. Once the reaction was complete, the resulting mixture was filtrated under reduced pressure using silica gel. Then, the filtrate was concentrated. A precipitate obtained by using dichloromethane and methanol was filtered, and the resulting solid was dried in a vacuum oven to thereby obtain 6.87 g of Intermediate 112001-e (yield: 86%).

LC-Mass (calculated value: 498.30 g/mol, found value: 498.67 (M+1))

### Synthesis of Intermediate 112001-f

4.00 g (8.04 mmol) of Intermediate 112001-e, 7.40 g (24.2 mmol) of 2,6-dibromo-4-(tert-butyl)aniline, 0.278 g (0.242 mmol) of Pd(PPh₃)₄, and 120 mL of THE were stirred together. Then, 60 mL of 2 M potassium carbonate (K₂CO₃) aqueous solution was added thereto, followed by stirring under reflux. Once the mixture was cooled to room temperature, an extraction process was performed using dichloromethane and distilled water, and the extracted organic layer was concentrated and adsorbed to silica gel. Then, a purification process was performed by using column chromatography charged with silica gel to thereby obtain 1.73 g of Intermediate 112001-f (yield: 36%).

LC-Mass (calculated value: 597.23 g/mol, found value: 597.20 (M+1))

### Synthesis of Intermediate 112001-g

1.70 g (2.84 mmol) of Intermediate 112001-f, 0.958 g (8.53 mmol) of potassium tert-butoxide (t-BuOK), and 20 mL of dimethyl sulfoxide (DMSO) were added to a round flask and stirred under reflux while heating. Once the reaction was complete, distilled water was added thereto, and an extraction process was performed using dichloromethane. Then, adsorption to silica gel occurred. Next, impurities were removed by using column chromatography and dried to thereby obtain 1.31 g of Intermediate 112001-g (yield: 80%).

LC-Mass (calculated value: 577.22 g/mol, found value: 577.86 (M+1))

### Synthesis of Intermediate 112001-h

1.30 g (2.25 mmol) of Intermediate 112001-g, 0.871 g (3.38 mmol) of 4,4,4′,4′,5,5,5′,5′-octamethyl-2,2′-bi(1,3,2-dioxaborolane), 0.662 g (6.75 mmol) of KOAc, and 0.049 g (0.068 mmol) of [1,1′-bis(diphenylphosphino)ferrocene]dichloropalladium(II), (PdCl₂(dppf)), and 30 mL of 1,4-dioxane were mixed together and stirred under reflux while heating. Once the reaction was complete, dichloromethane was added thereto, and filtration under reduced pressure was performed by using a filter charged with silica gel for concentration. Subsequently, a precipitate obtained by using dichloromethane and methanol was filtered, and the resulting solid was dried in a vacuum oven to thereby obtain 1.25 g of Intermediate 112001-h (yield: 89%).

LC-Mass (calculated value: 625.40 g/mol, found value: 625.10 (M+1))

### Synthesis of Intermediate 112001-i

0.550 g of Intermediate 112001-i was synthesized in substantially the same manner as in Synthesis of Intermediate 112001-c in Synthesis Example 1, except that 3,6-di-tert-butyl-1-(3,6-dibromo-2-iodophenyl)-9H-carbazole was used instead of Intermediate 112001-b (yield: 57%).

LC-Mass (calculated value: 1008.35 g/mol, found value: 1009.55 (M+1))

### Synthesis of Compound 112001

0.400 g (0.396 mmol) of Intermediate 112001-i, 0.075 g (0.396 mmol) of Cul, 0.071 g (0.396 mmol) of 1,10-phenanthroline, 0.336 g (1.58 mmol) of potassium phosphate, and 15 ml of DMF were heated and stirred. Once the reaction was complete, the mixture was heated and dissolved in 2 liters (L) of chloroform. Then, a filtration process was performed using silica gel and purified to thereby obtain 0.252 g of Compound 112001 (yield: 74%).

LC-Mass (calculated value: 847.4865 g/mol, found value: 847.4866 (M+1))

### Synthesis Example 2: Synthesis of Compound 112002

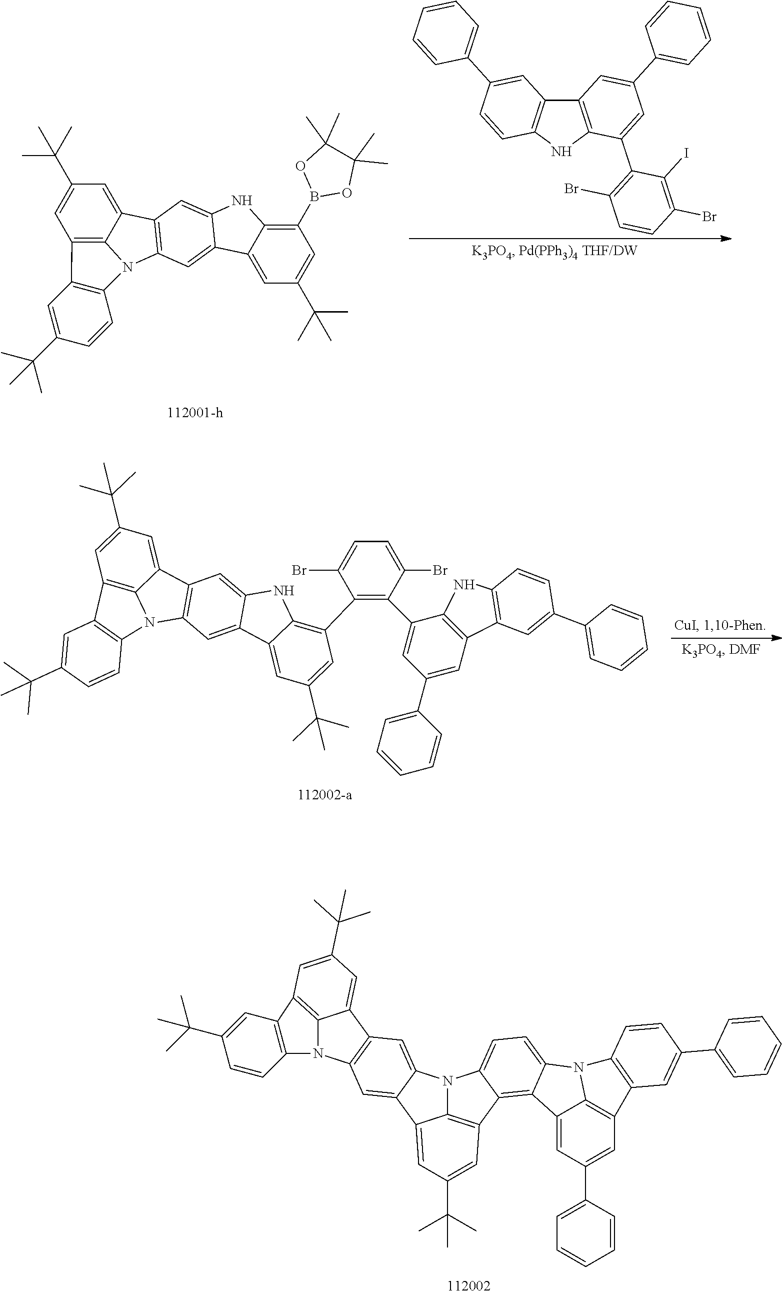 [SMILES: CC(C)(C)c1cc(B2OC(C)(C)C(C)(C)O2)c2[nH]c3cc4c5cc(C(C)(C)C)cc6c7cc(C(C)(C)C)ccc7n(c4cc3c2c1)c65 ; Brc1ccc(Br)c(-c2cc(-c3ccccc3)cc3c2[nH]c2ccc(-c4ccccc4)cc23)c1I ; CC(C)(C)c1cc(-c2c(Br)ccc(Br)c2-c2cc(-c3ccccc3)cc3c2[nH]c2ccc(-c4ccccc4)cc23)c2[nH]c3cc4c5cc(C(C)(C)C)cc6c7cc(C(C)(C)C)ccc7n(c4cc3c2c1)c65 ; CC(C)(C)c1ccc2c(c1)c1cc(C(C)(C)C)cc3c4cc5c(cc4n2c13)c1cc(C(C)(C)C)cc2c3c4c6cc(-c7ccccc7)cc7c8cc(-c9ccccc9)ccc8n(c4ccc3n5c12)c76]

### Synthesis of Intermediate 112002-a

0.581 g of Intermediate 112002-a was obtained in substantially the same manner as in Synthesis of Intermediate 112001-c in Synthesis Example 1 using 0.600 g (0.961 mmol) of Intermediate 112001-h, except that 0.979 g (1.44 mmol) of 1-(2,5-dibromo-4-iodophenyl)-3,6-diphenyl-9H-carbazole was used instead of the Intermediate, i.e., 3,6-di-t-butyl-1-(3,6-dibromo-2-iodophenyl)-9 H-carbazole (yield: 58%).

LC-Mass (calculated value: 1048.28 g/mol, found value: 1049.05 (M+1))

### Synthesis of Compound 112002

0.293 g of Compound 112002 was obtained in substantially the same manner in Synthesis of Compound 112001 in Synthesis Example 1, except that Intermediate 112002-a was used instead of Intermediate 112001-i (yield: 88%).

LC-Mass (calculated value: 887.4239 g/mol, found value: 887.4239 (M+1))

### Synthesis Example 3: Synthesis of Compound 115001

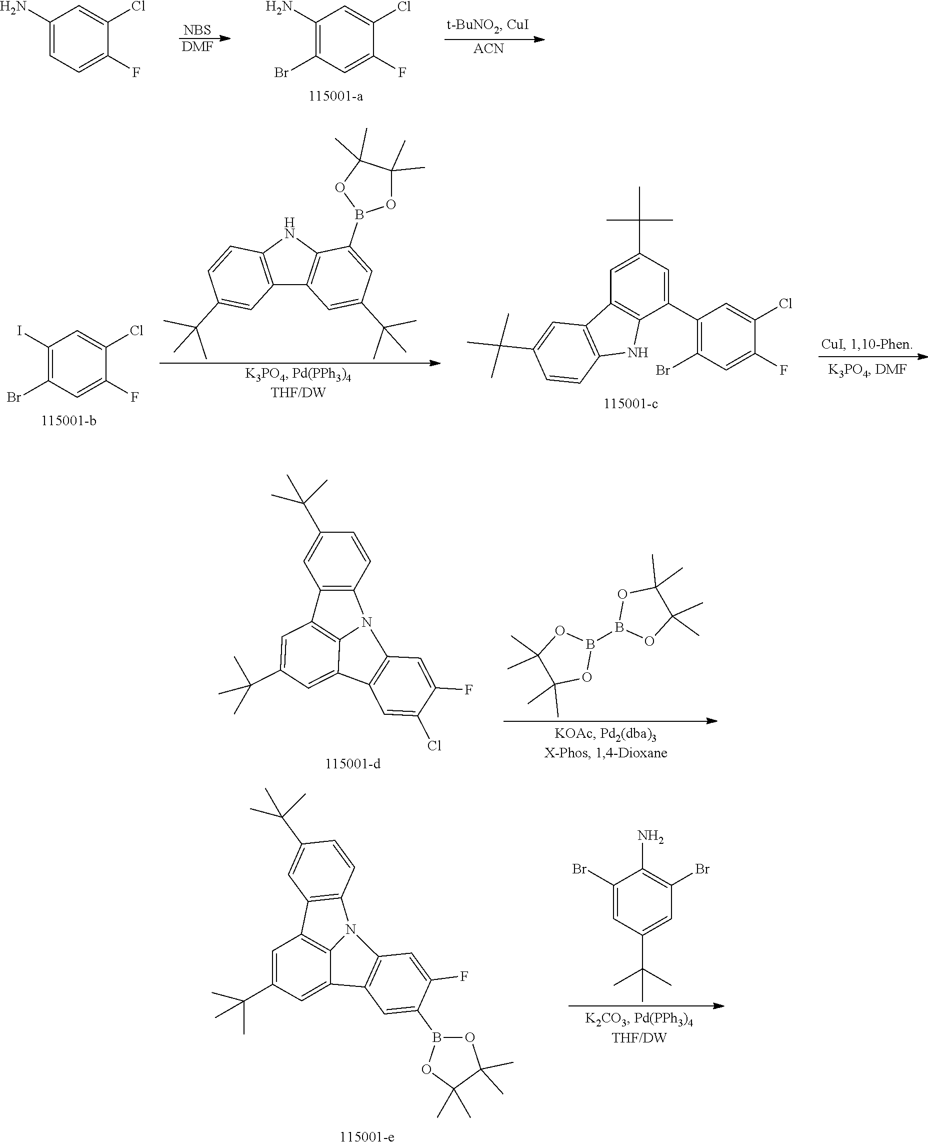

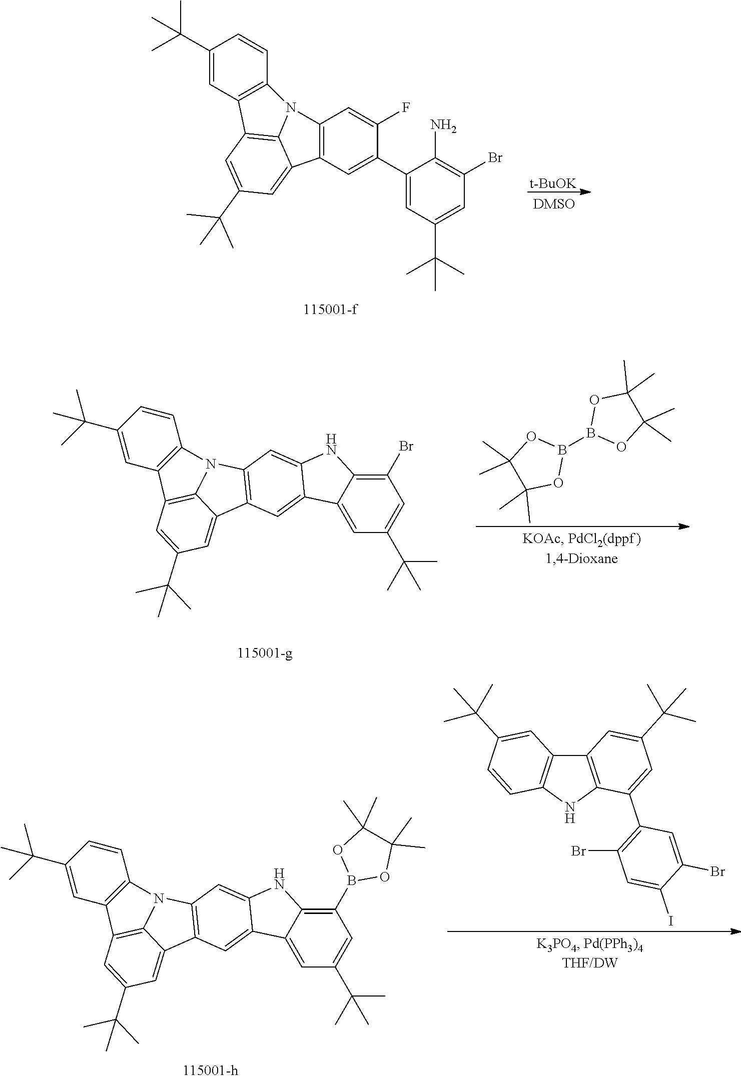

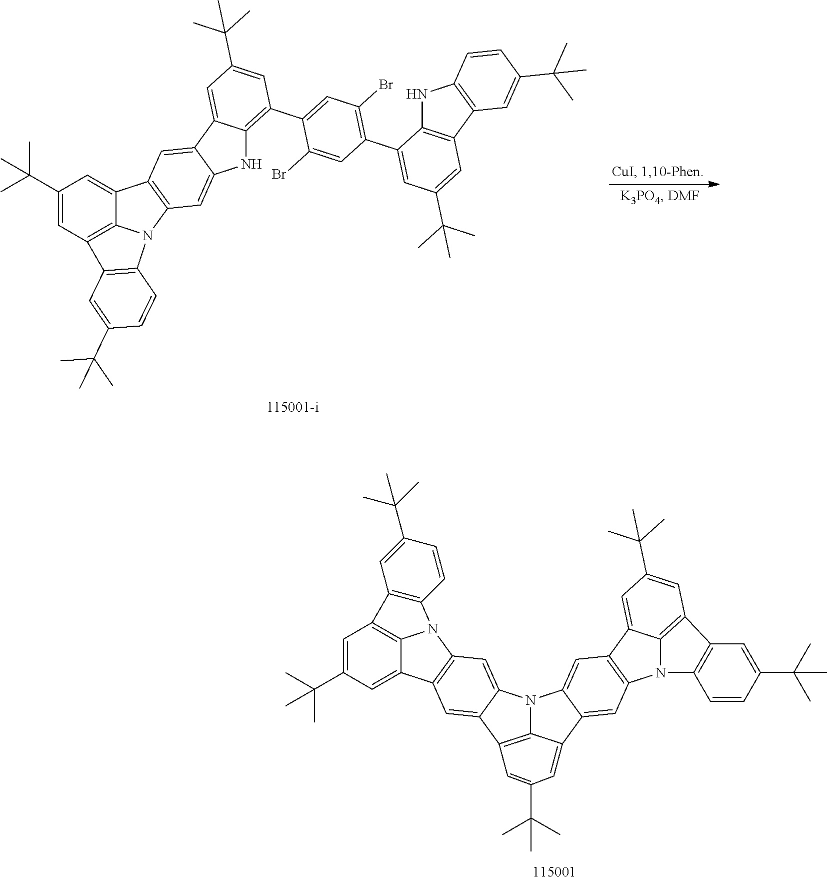

### Synthesis of Intermediate 115001-a

34.0 g of Intermediate 115001-a was obtained in substantially the same manner as in Synthesis of Intermediate 112001-a in Synthesis Example 1, except that 40.0 g (275 mmol) of 3-chloro-4-fluoroaniline was used instead of 4-chloro-3-fluoroaniline (yield: 55%).

LC-Mass (calculated value: 223.93 g/mol, found value: 223.17 (M+1))

### Synthesis of Intermediate 115001-b

9.32 g of Intermediate 115001-b was obtained in substantially the same manner as in Synthesis of Intermediate 112001-b in Synthesis Example 1, except that 15.0 g (66.8 mmol) of Intermediate 115001-a was used instead of Intermediate 112001-a (yield: 42%).

LC-Mass (calculated value: 334.81 g/mol, found value: 334.15 (M+1))

### Synthesis of Intermediate 115001-c

3.12 g of Intermediate 115001-c was obtained in substantially the same manner as in Synthesis of Intermediate 112001-c in Synthesis Example 1, except that Intermediate 115001-b was used instead of Intermediate 112001-b (yield: 43%).

LC-Mass (calculated value: 486.10 g/mol, found value: 486.62 (M+1))

### Synthesis of Intermediate 115001-d

2.43 g of Intermediate 115001-d was obtained in substantially the same manner as in Synthesis of Intermediate 112001-d in Synthesis Example 1, except that 3.00 g (6.16 mmol) of Intermediate 115001-c was used instead of Intermediate 112001-c (yield: 97%).

LC-Mass (calculated value: 405.17 g/mol, found value: 405.63 (M+1))

### Synthesis of Intermediate 115001-e

4.20 g of Intermediate 115001-e was obtained in substantially the same manner as in Synthesis of Intermediate 112001-e in Synthesis Example 1, except that 3.70 g (9.11 mmol) of Intermediate 115001-d was used instead of Intermediate 112001-d (yield: 93%).

LC-Mass (calculated value: 498.30 g/mol, found value: 498.89 (M+1))

### Synthesis of Intermediate 115001-f

1.30 g of Intermediate 115001-f was obtained in substantially the same manner as in Synthesis of Intermediate 112001-f in Synthesis Example 1, except that 2.00 g (4.02 mmol) of Intermediate 115001-e was used instead of Intermediate 112001-e (yield: 54%).

LC-Mass (calculated value: 597.23 g/mol, found value: 597.98 (M+1))

### Synthesis of Intermediate 115001-g

1.01 g of Intermediate 115001-g was obtained in substantially the same manner as in Synthesis of Intermediate 112001-g in Synthesis Example 1, except that Intermediate 115001-f was used instead of Intermediate 112001-f (yield: 69%).

LC-Mass (calculated value: 577.22 g/mol, found value: 577.20 (M+1))

### Synthesis of Intermediate 115001-h

0.972 g of Intermediate 115001-h was obtained in substantially the same manner as in Synthesis of Intermediate 112001-h in Synthesis Example 1, except that 1.00 g (1.73 mmol) of Intermediate 115001-g was used instead of Intermediate 112001-g (yield: 90%).

LC-Mass (calculated value: 625.40 g/mol, found value: 625.28 (M+1))

### Synthesis of Intermediate 115001-i

0.440 g of Intermediate 115001-i was obtained in substantially the same manner as in Synthesis of Intermediate 112001-i in Synthesis Example 1, except that Intermediate 115001-h was used (yield: 45%).

LC-Mass (calculated value: 1008.35 g/mol, found value: 1007.95 (M+1))

### Synthesis of Compound 115001

0.281 g of Compound 115001 was obtained in substantially the same manner as in Synthesis of Compound 112001 in Synthesis Example 1, except that 0.400 g (0.396 mmol) of Intermediate 115001-i was used instead of Intermediate 112001-i (yield: 83%).

LC-Mass (calculated value: 847.4865 g/mol, found value: 847.4870 (M+1))

### Synthesis Example 4: Synthesis of Compound 115002

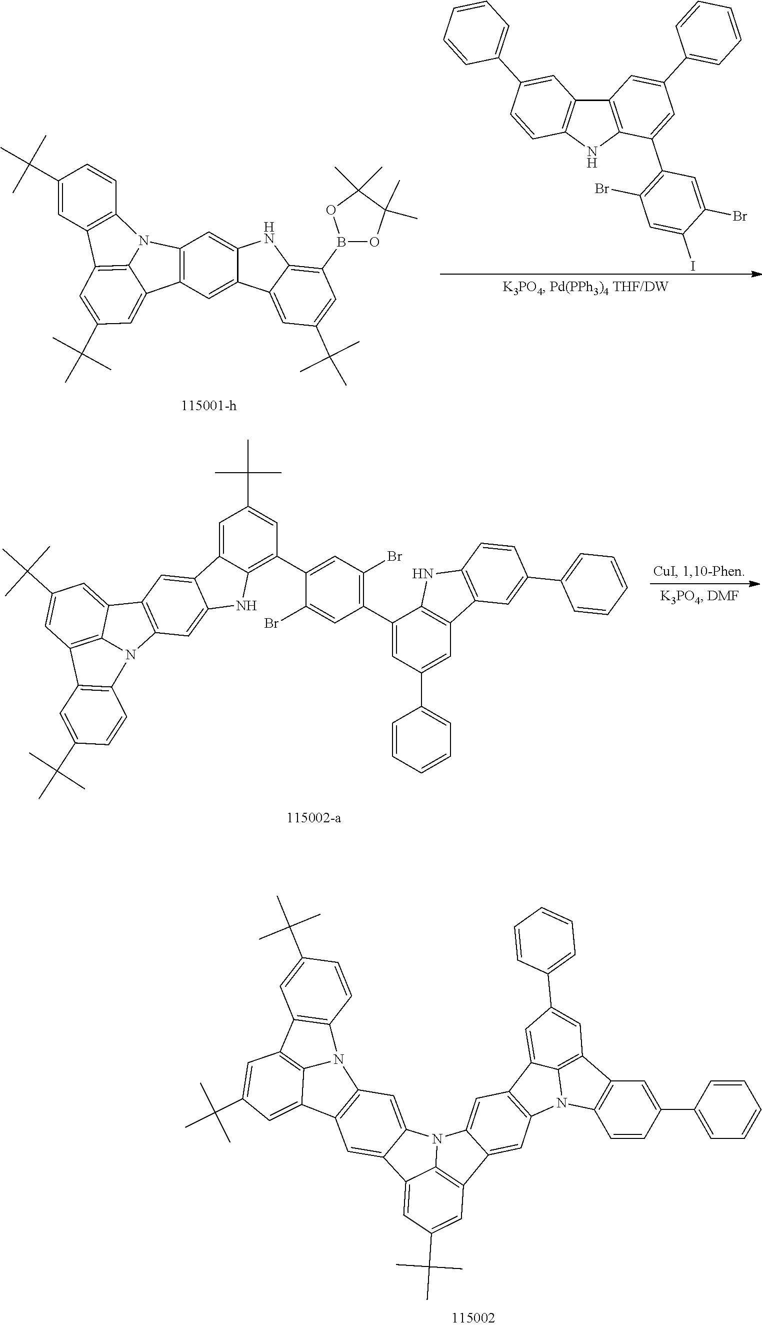 [SMILES: CC(C)(C)c1cc(B2OC(C)(C)C(C)(C)O2)c2[nH]c3cc4c(cc3c2c1)c1cc(C(C)(C)C)cc2c3cc(C(C)(C)C)ccc3n4c21 ; Brc1cc(-c2cc(-c3ccccc3)cc3c2[nH]c2ccc(-c4ccccc4)cc23)c(Br)cc1I ; CC(C)(C)c1cc(-c2cc(Br)c(-c3cc(-c4ccccc4)cc4c3[nH]c3ccc(-c5ccccc5)cc34)cc2Br)c2[nH]c3cc4c(cc3c2c1)c1cc(C(C)(C)C)cc2c3cc(C(C)(C)C)ccc3n4c21 ; CC(C)(C)c1ccc2c(c1)c1cc(C(C)(C)C)cc3c4cc5c6cc(C(C)(C)C)cc7c8cc9c(cc8n(c5cc4n2c13)c76)c1cc(-c2ccccc2)cc2c3cc(-c4ccccc4)ccc3n9c21]

### Synthesis of Intermediate 115002-a

0.516 g of Intermediate 115002-a was obtained in substantially the same manner as in Synthesis of Intermediate 112002-a in Synthesis Example 2, except that 0.600 g (0.961 mmol) of Intermediate 115001-h was used as a start material (yield: 51%).

LC-Mass (calculated value: 1048.28 g/mol, found value: 1047.40 (M+1))

### Synthesis of Compound 115002

0.310 g of Compound 115002 was obtained in substantially the same manner as in Synthesis of Compound 112001 in Synthesis Example 1, except that Intermediate 115002-a was used (yield: 92%).

LC-Mass (calculated value: 887.4239 g/mol, found value: 887.4252 (M+1))

### Synthesis Example 5: Synthesis of Compound 115003

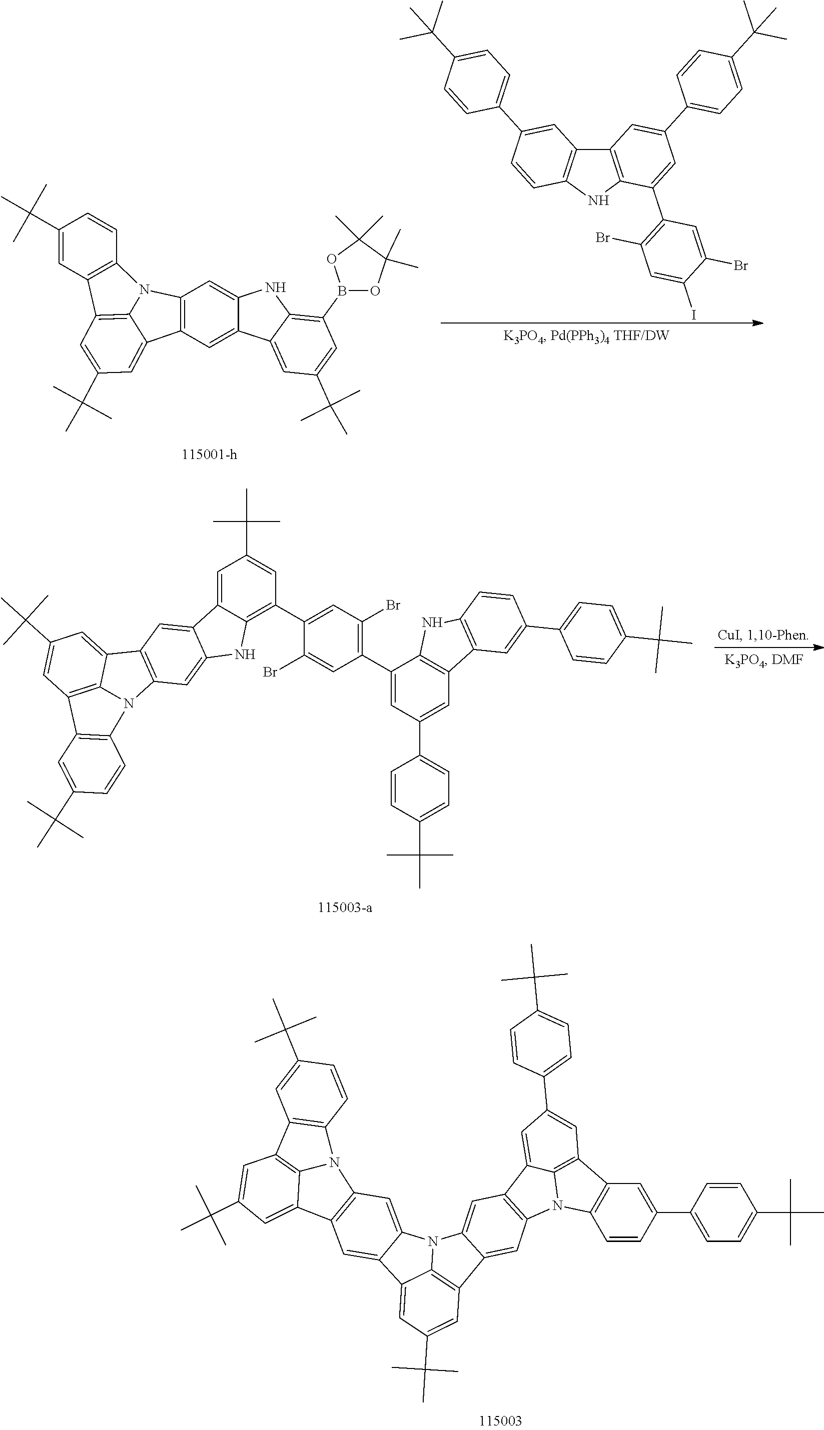

### Synthesis of Intermediate 115003-a

0.536 g of Intermediate 115003-a was obtained in substantially the same manner as in Synthesis of Intermediate 112001-a in Synthesis Example 1, except that 3,6-bis(4-(tert-butyl)phenyl)-1-(2,5-dibromo-4-iodophenyl)-9H-carbazole was used instead of 3,6-di-t-butyl-1-(3,6-dibromo-2-iodophenyl)-9H-cabazole to react with 0.600 g of Intermediate 115001-h (0.961 mmol) (yield: 48%).

LC-Mass (calculated value: 1160.41 g/mol, found value: 1161.21 (M+1))

### Synthesis of Compound 115003

0.387 g of Compound 115003 was obtained in substantially the same manner as in Synthesis of Compound 112001 in Synthesis Example 1, except that Intermediate 115003-a was used in reaction (yield: 90%).

LC-Mass (calculated value: 1000.5570 g/mol, found value: 1000.5573 (M+1))

### Synthesis Example 6: Synthesis of Compound 115004

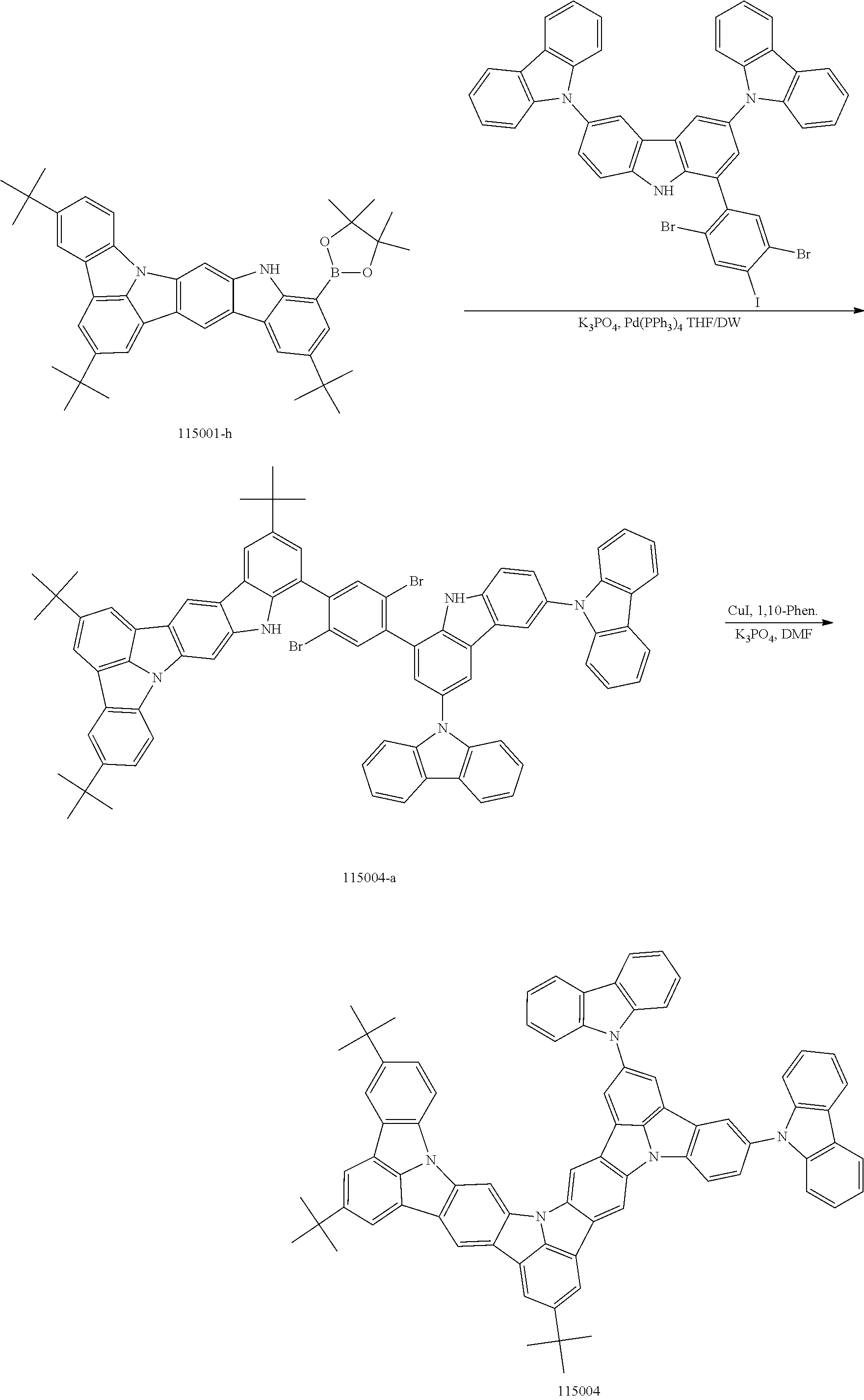 [SMILES: CC(C)(C)c1cc(B2OC(C)(C)C(C)(C)O2)c2[nH]c3cc4c(cc3c2c1)c1cc(C(C)(C)C)cc2c3cc(C(C)(C)C)ccc3n4c21 ; Brc1cc(-c2cc(-n3c4ccccc4c4ccccc43)cc3c2[nH]c2ccc(-n4c5ccccc5c5ccccc54)cc23)c(Br)cc1I ; CC(C)(C)c1cc(-c2cc(Br)c(-c3cc(-n4c5ccccc5c5ccccc54)cc4c3[nH]c3ccc(-n5c6ccccc6c6ccccc65)cc34)cc2Br)c2[nH]c3cc4c(cc3c2c1)c1cc(C(C)(C)C)cc2c3cc(C(C)(C)C)ccc3n4c21 ; CC(C)(C)c1ccc2c(c1)c1cc(C(C)(C)C)cc3c4cc5c6cc(C(C)(C)C)cc7c8cc9c(cc8n(c5cc4n2c13)c76)c1cc(-n2c3ccccc3c3ccccc32)cc2c3cc(-n4c5ccccc5c5ccccc54)ccc3n9c21]

### Synthesis of Intermediate 115004-a

0.507 g of Intermediate 115004-a was synthesized in substantially the same manner as in Synthesis of Intermediate 112001-a in Synthesis Example 1, except that Intermediate 1′-(2,5-dibromo-4-iodophenyl)-9′H-9,3′:6′,9″-tert-carbazole was used instead of Intermediate 3,6-di-t-butyl-1-(3,6-dibromo-2-iodophenyl)-9H-carbazole to react with Intermediate 115001-h (0.600 g, 0.961 mmol), followed by purification (yield: 43%).

LC-Mass (calculated value: 1226.34 g/mol, found value: 1225.88 (M+1))

### Synthesis of Compound 115004

0.395 g of Compound 115004 was obtained in substantially the same manner as in Synthesis of Compound 112001 in Synthesis Example 1, except that Intermediate 115004-a was used in reaction (yield: 91%).

LC-Mass (calculated value: 1066.4849 g/mol, found value: 1066.4848 (M+1))

### Synthesis Example 7: Synthesis of Compound 115005

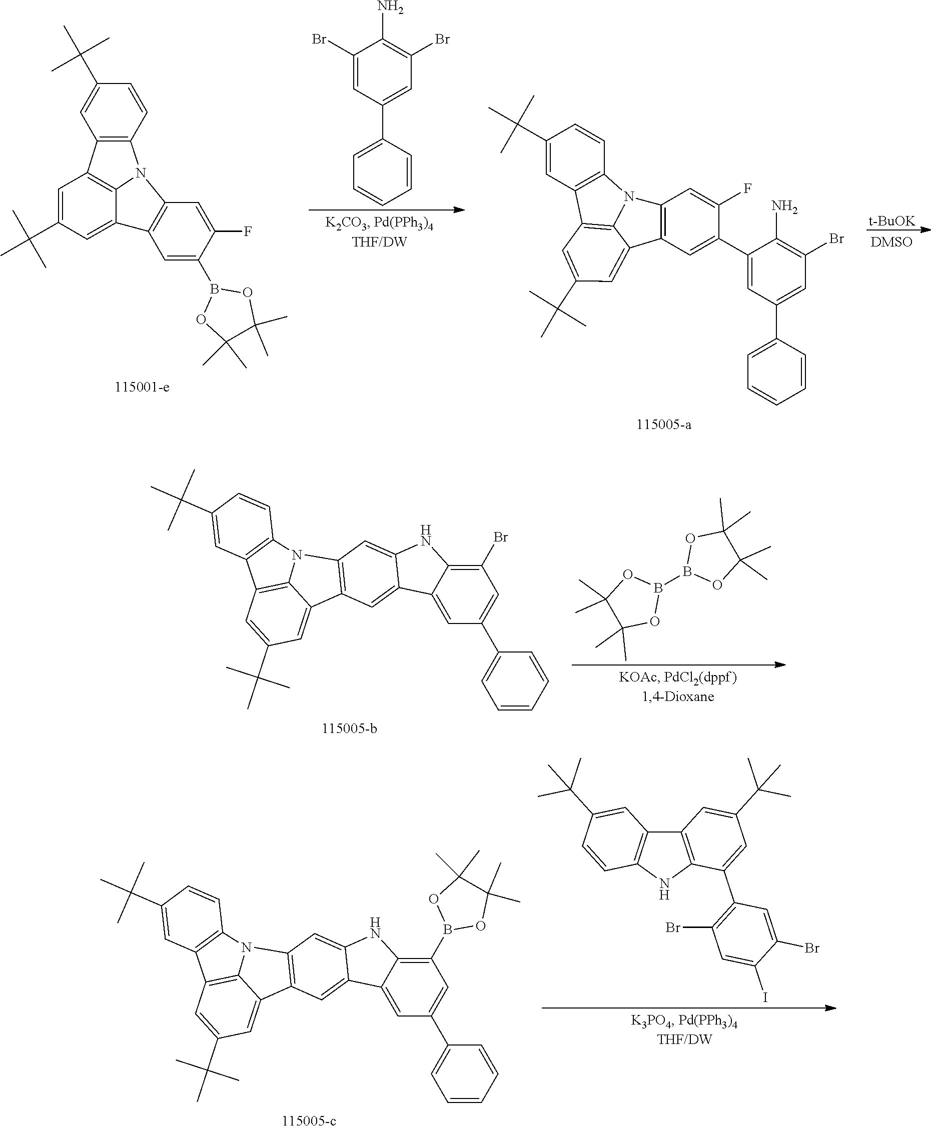

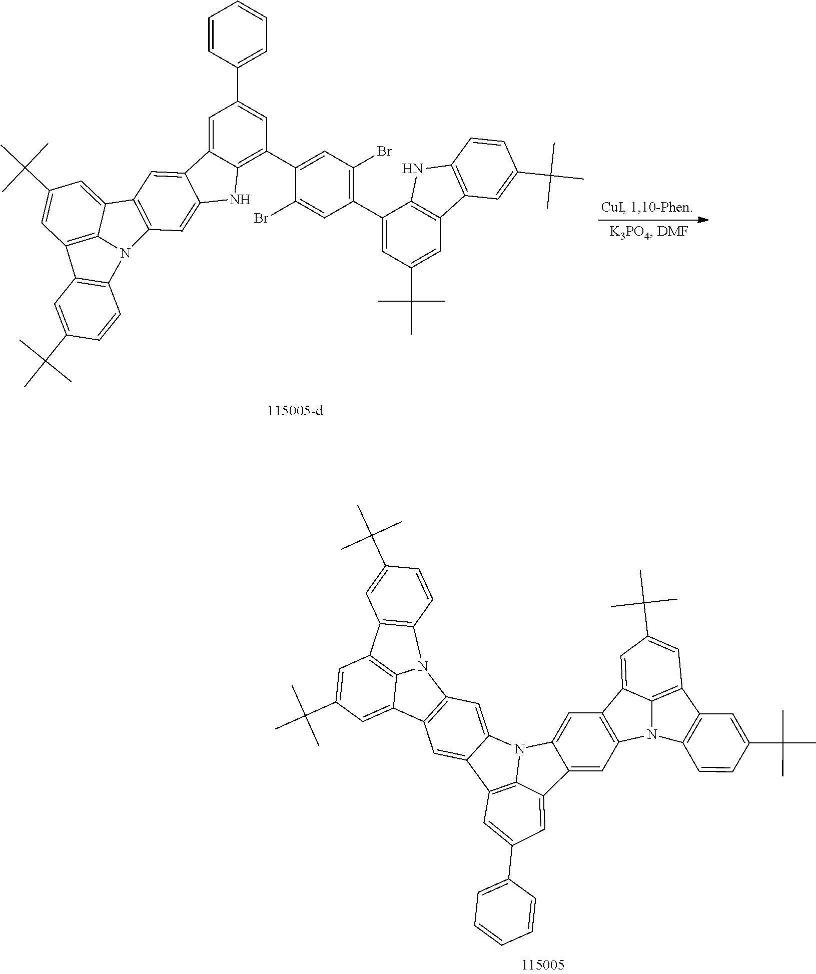

### Synthesis of Intermediate 115005-a

1.17 g of Intermediate 115005-a was obtained in substantially the same manner as in Synthesis of Intermediate 112001-f in Synthesis Example 1, except that 3.96 g (12.1 mmol) of 3,5-dibromo-[1,1′-biphenyl]-4-amine was used instead of Intermediate 2,6-dibromo-4-(t-butyl)aniline to react with Intermediate 115001-e (yield: 47%).

LC-Mass (calculated value: 617.20 g/mol, found value: 617.44 (M+1))

### Synthesis of Intermediate 115005-b

1.37 g of Intermediate 115005-b was obtained in substantially the same manner as in Synthesis of Intermediate 112001-g in Synthesis Example 1, except that Intermediate 115005-a was used (yield: 71%).

LC-Mass (calculated value: 597.19 g/mol, found value: 597.72 (M+1))

### Synthesis of Intermediate 115005-c

0.949 g of Intermediate 115005-c was obtained in substantially the same manner as in Synthesis of Intermediate 112001-h in Synthesis Example 1, except that Intermediate 115005-b was used in reaction (yield: 88%).

LC-Mass (calculated value: 645.37 g/mol, found value: 646.01 (M+1))

### Synthesis of Intermediate 115005-d

0.751 g of Intermediate 115005-d was obtained in substantially the same manner as in Synthesis of Intermediate 112001-c in Synthesis Example 1, except that Intermediate 115005-c was used as a start material (yield: 61%).

LC-Mass (calculated value: 1028.32 g/mol, found value: 1029.22 (M+1))

### Synthesis of Compound 115005

0.421 g of Compound 115005 was obtained in substantially the same manner as in Synthesis of Compound 112001 in Synthesis Example 1, except that Intermediate 115005-d was used in reaction (yield: 86%).

LC-Mass (calculated value: 868.4631 g/mol, found value: 868.4633 (M+1))

### Synthesis Example 8: Synthesis of Compound 115006

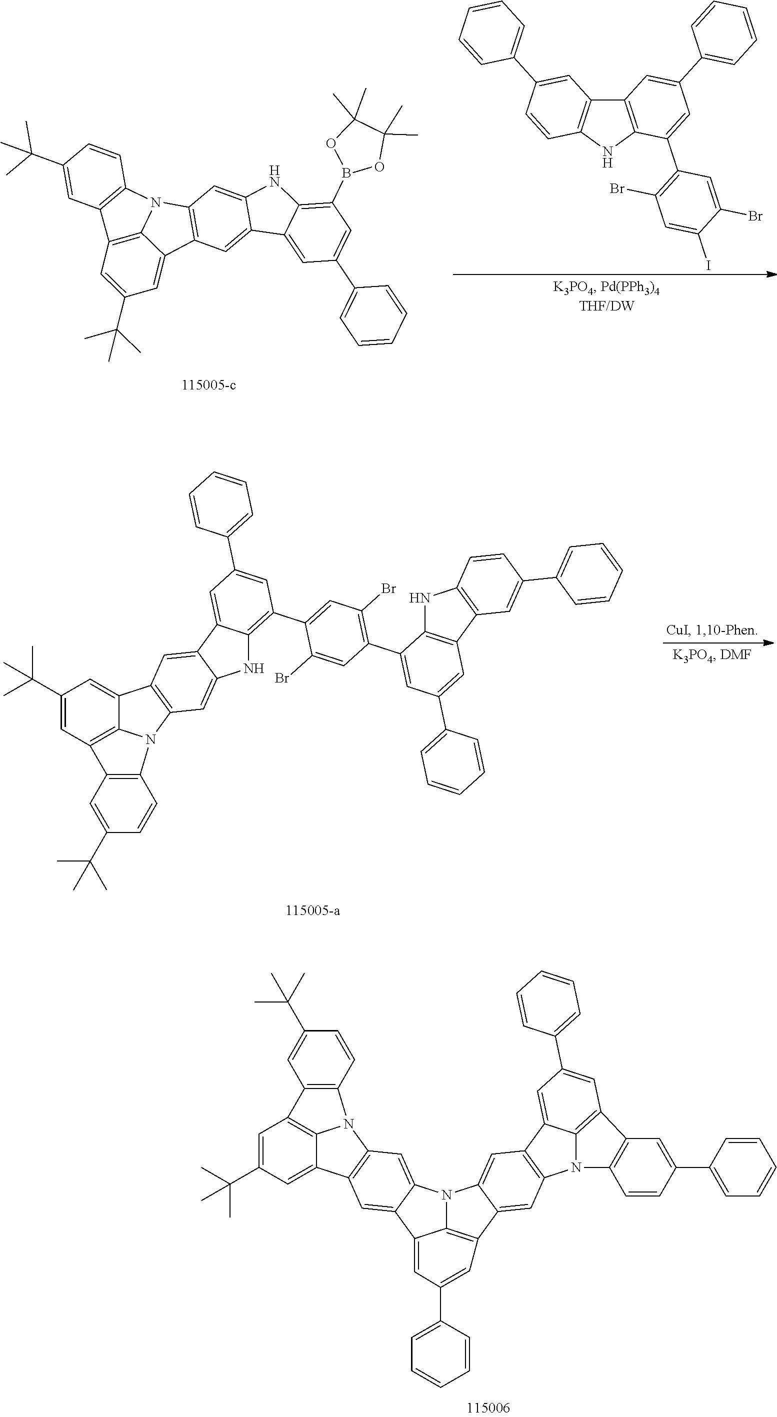 [SMILES: CC(C)(C)c1ccc2c(c1)c1cc(C(C)(C)C)cc3c4cc5c(cc4n2c13)[nH]c1c(B2OC(C)(C)C(C)(C)O2)cc(-c2ccccc2)cc15 ; Brc1cc(-c2cc(-c3ccccc3)cc3c2[nH]c2ccc(-c4ccccc4)cc23)c(Br)cc1I ; CC(C)(C)c1ccc2c(c1)c1cc(C(C)(C)C)cc3c4cc5c(cc4n2c13)[nH]c1c(-c2cc(Br)c(-c3cc(-c4ccccc4)cc4c3[nH]c3ccc(-c6ccccc6)cc34)cc2Br)cc(-c2ccccc2)cc15 ; CC(C)(C)c1ccc2c(c1)c1cc(C(C)(C)C)cc3c4cc5c6cc(-c7ccccc7)cc7c8cc9c(cc8n(c5cc4n2c13)c76)c1cc(-c2ccccc2)cc2c3cc(-c4ccccc4)ccc3n9c21]

### Synthesis of Intermediate 115006-a

1.28 g of Intermediate 115006-a was obtained in substantially the same manner as in Synthesis of Intermediate 112002-a in Synthesis Example 2, except that Intermediate 115005-b was used in reaction (yield: 52%).

LC-Mass (calculated value: 1068.25 g/mol, found value: 1067.78 (M+1))

### Synthesis of Compound 115006

0.243 g of Compound 115006 was obtained in substantially the same manner as in Synthesis of Compound 112001 in Synthesis Example 1, except that Intermediate 115006-a was used in reaction (yield: 95%).

LC-Mass (calculated value: 908.4005 g/mol, found value: 908.4010 (M+1))

### Synthesis Example 9: Synthesis of Compound 115007

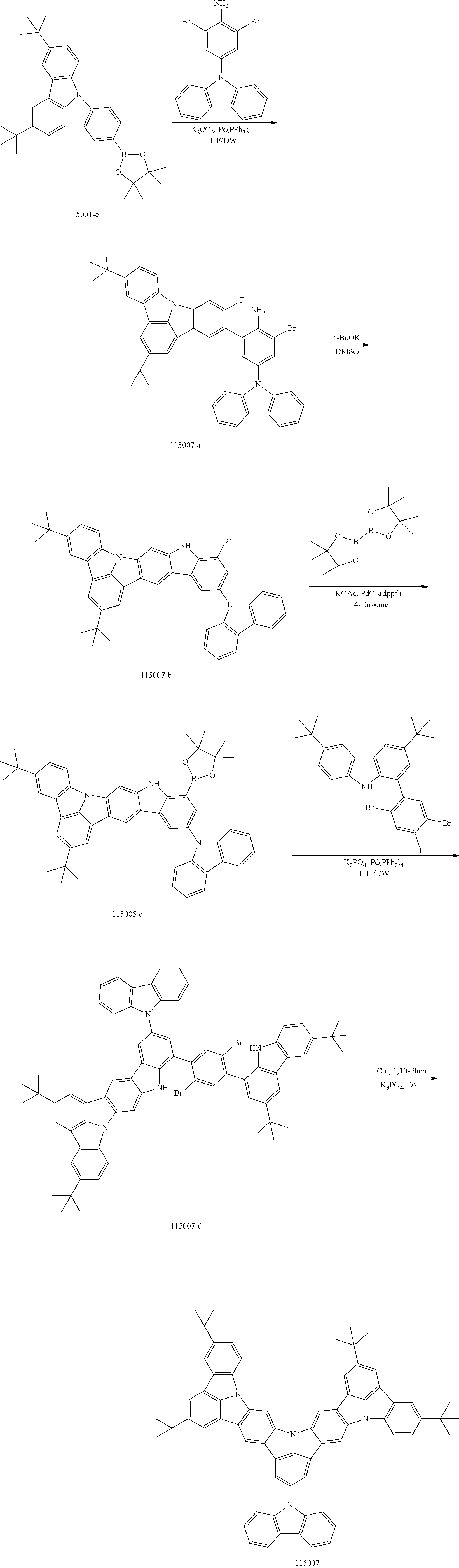

### Synthesis of Intermediate 115007-a

1.08 g of Intermediate 115007-a was obtained in substantially the same manner as in Synthesis of 112001-f in Synthesis Example 1, except that 2,6-dibromo-4-(9H-carbazole-9-yl)aniline was used instead of Intermediate 2,6-dibromo-4-(tert-butyl)aniline to react with Intermediate 115001-e, followed by purification (yield: 38%).

LC-Mass (calculated value: 706.22 g/mol, found value: 707.01 (M+1))

### Synthesis of Intermediate 115007-b

1.53 g of Intermediate 115007-b was obtained in substantially the same manner as in Synthesis of Intermediate 112001-g in Synthesis Example 1, except that Intermediate 115007-a was used in reaction (yield: 63%).

LC-Mass (calculated value: 686.22 g/mol, found value: 686.75 (M+1))

### Synthesis of Intermediate 115007-c

1.13 g of Intermediate 115007-c was obtained in substantially the same manner as in Synthesis of Intermediate 112001-h in Synthesis Example 1, except that Intermediate 115007-b was used (yield: 85%).

LC-Mass (calculated value: 734.39 g/mol, found value: 734.52 (M+1))

### Synthesis of Intermediate 115007-d

0.821 g of Intermediate 115007-d was obtained in substantially the same manner as in Synthesis of Intermediate 112001-c in Synthesis Example 1, except that Intermediate 115007-c was used in reaction (yield: 48%).

LC-Mass (calculated value: 1117.34 g/mol, found value: 1118.52 (M+1))

### Synthesis of Compound 115007

0.642 g of Compound 115007 was obtained in substantially the same manner as in Synthesis of Compound 112001 in Synthesis Example 1, except that Intermediate 115007-d was used in reaction (yield: 81%).

LC-Mass (calculated value: 957.4896 g/mol, found value: 957.4900 (M+1))

### Synthesis Example 10: Synthesis of Compound 115008

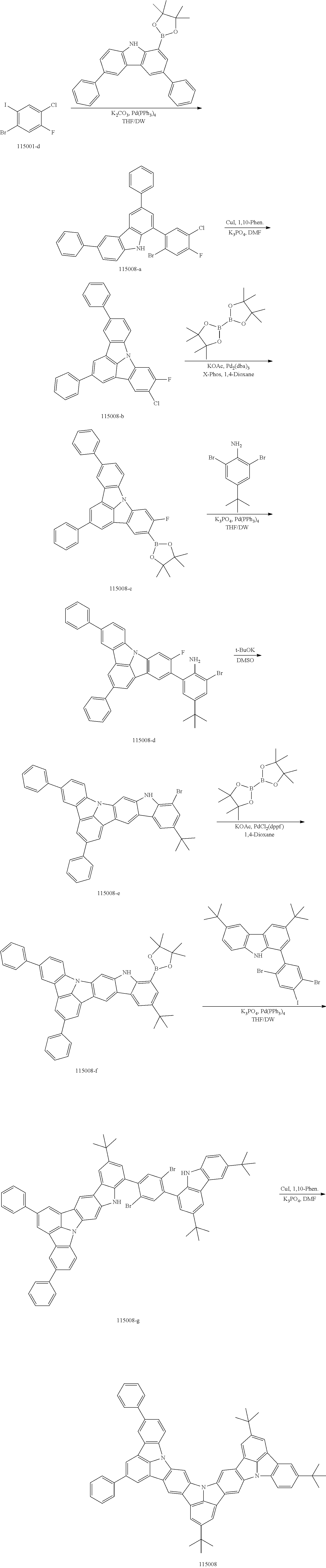

### Synthesis of Intermediate 115008-a

1.35 g of Intermediate 115008-a was obtained in substantially the same manner as in Synthesis of Intermediate 112001-c in Synthesis Example 1, except that 3,6-diphenyl-1-(4,4,5,5-tetramethyl-1,3,2-dioxaborolan-2-yl)-9H-carbazole was used as a start material instead of 3,6-di-tert-butyl-1-(4,4,5,5-tetramethyl-1,3,2-dioxaborolan-2-yl)—9H-carbazole to react with Intermediate 115001-b (yield: 42%).

LC-Mass (calculated value: 526.04 g/mol, found value: 527.00 (M+1))

### Synthesis of Intermediate 115008-b

3.5213 g of Intermediate 115008-b was obtained in substantially the same manner as in Synthesis of Intermediate 112001-c in Synthesis Example 1, except that Intermediate 115008-a was used in reaction (yield: 91%).

LC-Mass (calculated value: 446.11 g/mol, found value: 446.29 (M+1))

### Synthesis of Intermediate 115008-c

2.13 g of Intermediate 115008-c was obtained in substantially the same manner as in Synthesis of Intermediate 112001-e in Synthesis Example 1, except that Intermediate 115008-b was used instead of Intermediate 112001-d (yield: 71%).

LC-Mass (calculated value: 538.24 g/mol, found value: 538.78 (M+1))

### Synthesis of Intermediate 115008-d

1.73 g of Intermediate 115008-d was obtained in substantially the same manner as in Synthesis of Intermediate 112001-f in Synthesis Example 1, except that Intermediate 115008-c was used (yield: 45%).

LC-Mass (calculated value: 637.17 g/mol, found value: 638.05 (M+1))

### Synthesis of Intermediate 115008-e

1.32 g of Intermediate 115008-e was obtained in substantially the same manner as in Synthesis of Intermediate 112001-g in Synthesis Example 1, except that Intermediate 115008-d was used in reaction (yield: 65%).

LC-Mass (calculated value: 617.16 g/mol, found value: 617.85 (M+1))

### Synthesis of Intermediate 115008-f

1.02 g of Intermediate 115008-f was obtained in substantially the same manner as in Synthesis of Intermediate 112001-h in Synthesis Example 1, except that Intermediate 115008-e was used in reaction (yield: 81%).

LC-Mass (calculated value: 665.33 g/mol, found value: 666.11 (M+1))

### Synthesis of Intermediate 115008-g

Intermediate 115008-f was subjected to synthesis in substantially the same manner as in Synthesis of Intermediate 112001-c in Synthesis Example 1. Once the reaction and purification were complete, 1.00 g of Intermediate 115008-g was obtained (yield: 47%).

LC-Mass (calculated value: 1048.28 g/mol, found value: 1047.99 (M+1))

### Synthesis of Compound 115008

0.441 g of Compound 115008 was obtained in substantially the same manner as in Synthesis of Compound 112001 in Synthesis Example 1 (yield: 83%).

LC-Mass (calculated value: 888.4318 g/mol, found value: 888.4320 (M+1))

### Synthesis Example 11: Synthesis of Compound 115009

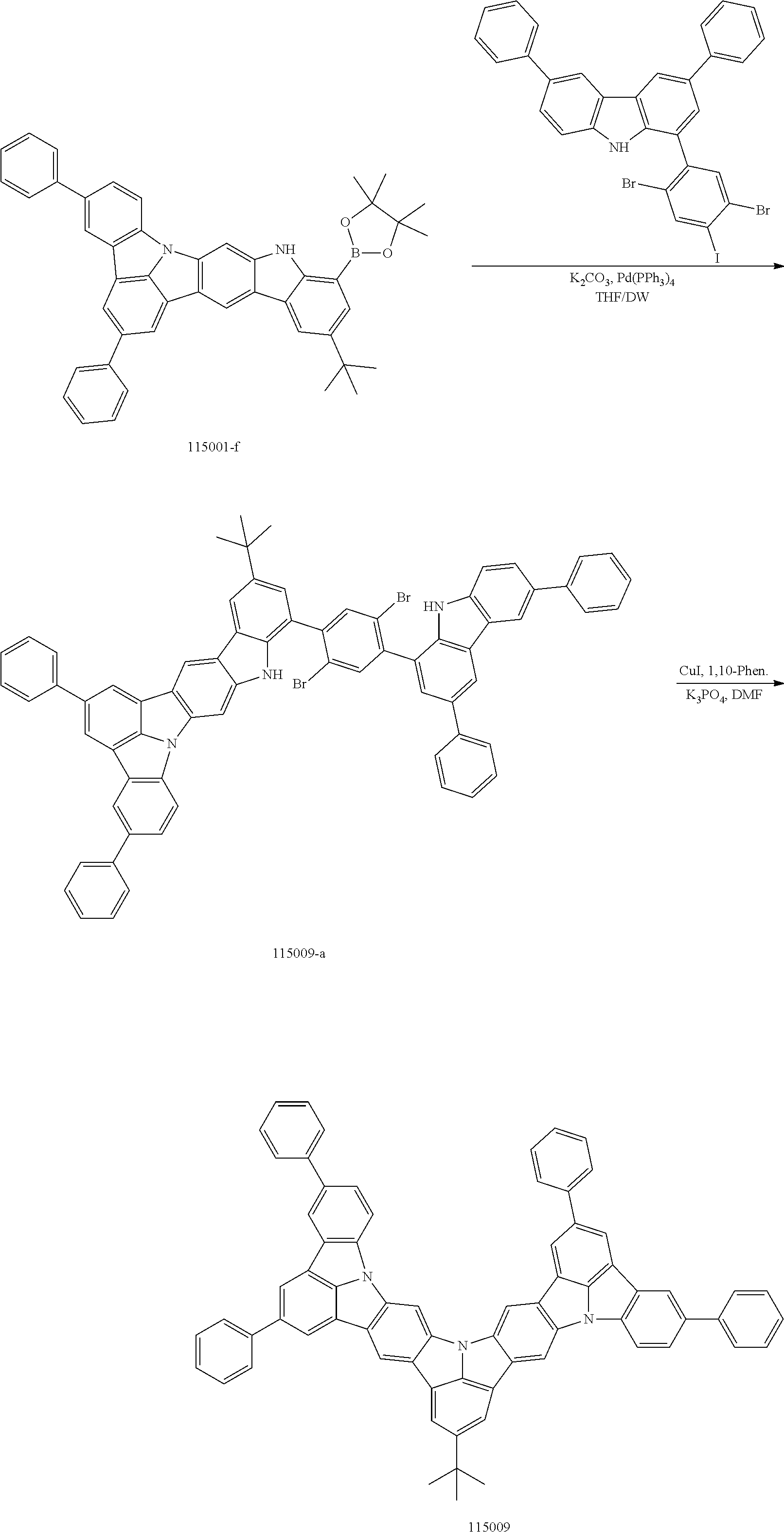 [SMILES: CC(C)(C)c1cc(B2OC(C)(C)C(C)(C)O2)c2[nH]c3cc4c(cc3c2c1)c1cc(-c2ccccc2)cc2c3cc(-c5ccccc5)ccc3n4c21 ; Brc1cc(-c2cc(-c3ccccc3)cc3c2[nH]c2ccc(-c4ccccc4)cc23)c(Br)cc1I ; CC(C)(C)c1cc(-c2cc(Br)c(-c3cc(-c4ccccc4)cc4c3[nH]c3ccc(-c5ccccc5)cc34)cc2Br)c2[nH]c3cc4c(cc3c2c1)c1cc(-c2ccccc2)cc2c3cc(-c5ccccc5)ccc3n4c21 ; CC(C)(C)c1cc2c3cc4c(cc3n3c5cc6c(cc5c(c1)c23)c1cc(-c2ccccc2)cc2c3cc(-c5ccccc5)ccc3n6c21)c1cc(-c2ccccc2)cc2c3cc(-c5ccccc5)ccc3n4c21]

### Synthesis of Intermediate 115009-a

Intermediate 115008-f was subjected to synthesis in substantially the same manner as in Synthesis of Intermediate 112002-a in Synthesis Example 2. 1.23 g of Intermediate 115009-a was obtained after purification completed (yield: 44%).

LC-Mass (calculated value: 1088.22 g/mol, found value: 1089.11 (M+1))

### Synthesis of Compound 115009

0.333 g of Compound 115009 was obtained in substantially the same manner as in Synthesis of Compound 112001 in Synthesis Example 1, except that Intermediate 115009-a was used in reaction (yield: 91%).

LC-Mass (calculated value: 928.3692 g/mol, found value: 928.3690 (M+1))

### Synthesis Example 12: Synthesis of Compound 121001

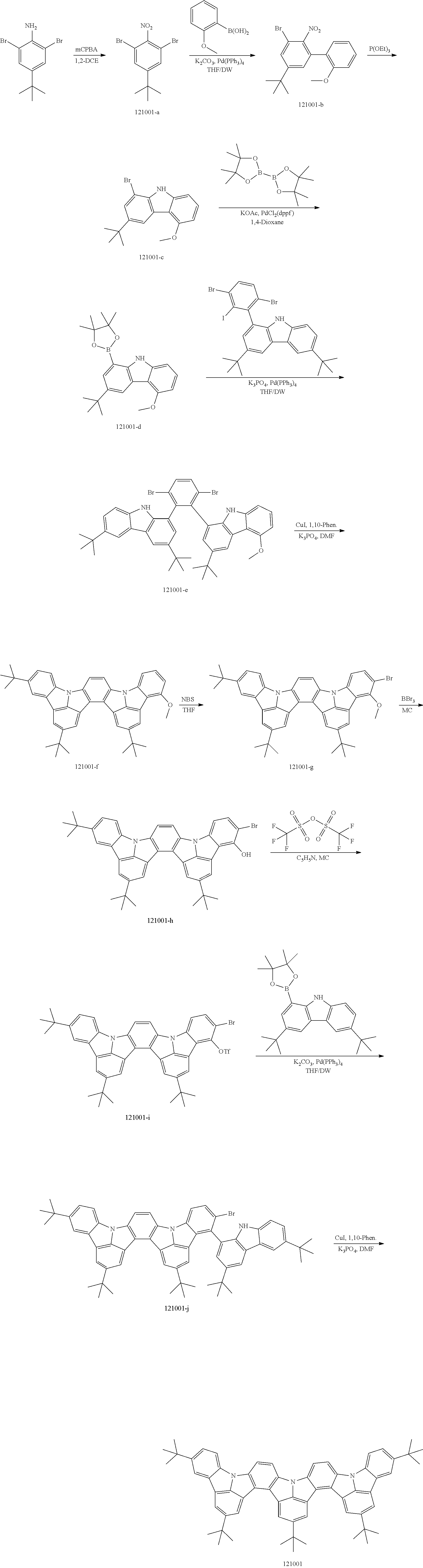

### Synthesis of Intermediate 121001-a

100 g (326 mmol) of 2,6-dibromo-4-(tert-butyl)aniline, 276 g (1.30 mol) of m-chloroperbenzoic acid (mCPBA), and 1,500 mL of 1,2-dichloroethane (1,2-DCE) were added to a round flask and stirred. After heating for reflux and cooling for 16 hours, a sodium thiosulfate aqueous solution was added thereto. Then an extraction process was performed using dichloromethane and distilled water. The extracted organic layer was purified using column chromatography and dried to thereby obtain 62.1 g of Intermediate 121001-a (yield: 56%).

LC-Mass (calculated value: 335.92 g/mol, found value: 336.02 (M+1)) Synthesis of Intermediate 121001-b

12.5 g of Intermediate 121001-b was obtained in substantially the same manner as in Synthesis of Intermediate 112001-f in Synthesis Example 1, except that 15.0 g (98.9 mmol) of (2-methoxyphenyl)boronic acid was used as a start material instead of 50.0 g (148 mmol) of Intermediate 121001-a (yield: 35%).

LC-Mass (calculated value: 364.05 g/mol, found value: 365.11 (M+1)) Synthesis of Intermediate 121001-c

20.0 g (54.9 mmol) of Intermediate 121001-b and 200 mL of triethyl phosphite were stirred in a round flask and heated for 3 days at a temperature of 200° C. to allow a reaction to occur. Once the reaction was complete, 1,000 mL of ethanol was added thereto, followed by stirring for 3 hours. After an extraction process was complete, separation and purification were performed through column chromatography to remove impurities. 4.01 g of Intermediate 121001-c was obtained after drying (yield: 22%).

LC-Mass (calculated value: 332.07 g/mol, found value: 332.55 (M+1))

### Synthesis of Intermediate 121001-d

15.0 g (45.1 mmol) of Intermediate 121001-c was used in substantially the same manner as in Synthesis of Intermediate 112001-h in Synthesis Example 1. 15.3 g of Intermediate 121001-d was obtained after purification completed (yield: 89%).

LC-Mass (calculated value: 380.24 g/mol, found value: 379.98 (M+1)) Synthesis of Intermediate 121001-e

8.27 g of Intermediate 121001-e was obtained in substantially the same manner as in Synthesis of Intermediate 112001-i in Synthesis Example 1, except that Intermediate 121001-d was used instead of Intermediate 112001-h (yield: 41%).

LC-Mass (calculated value: 763.19 g/mol, found value: 763.15 (M+1))

### Synthesis of Intermediate 121001-f

5.91 g of Intermediate 121001-f was obtained in substantially the same manner as in Synthesis of Compound 112001 in Synthesis Example 1, except that Intermediate 121001-d was used in reaction (yield: 94%).

LC-Mass (calculated value: 603.34 g/mol, found value: 604.21 (M+1))

### Synthesis of Intermediate 121001-g

A reaction was performed in substantially the same manner as in Synthesis of Intermediate 112001-a in Synthesis Example 1, except that THE was used instead of DMF as a solvent, and Intermediate 121001-f was used in reaction. 1.62 g of Intermediate 121001-g was obtained (yield: 48%).

LC-Mass (calculated value: 681.25 g/mol, found value: 681.77 (M+1))

### Synthesis of Intermediate 121001-h

5.00 g (7.33 mmol) of Intermediate 121001-g was dissolved in 100 mL of dichloromethane and cooled to −78° C. Then, 3.67 g (14.7 mmol) of boron tribromide (BBr₃) was slowly added thereto. 24 hours after stirring, distilled water was added thereto to quench excess BBr₃. Then, neutralization was performed until pH 7 was reacted by using a sodium hydroxide aqueous solution. An extraction process was performed using dichloromethane and distilled water. Then, precipitation was performed using n-hexane, followed by filtering and drying. 3.12 g of Intermediate 121001-h was thus obtained (yield: 64%).

LC-Mass (calculated value: 667.23 g/mol, found value: 667.01 (M+1))

### Synthesis of Intermediate 121001-i

12.0 g (18.0 mmol) of Intermediate 121001-h, 2.84 g (35.9 mmol) of pyridine, and 120 mL of dichloromethane were added to a round flask, and the temperature was adjusted to 0° C. 6.08 g (21.6 mmol) of triflic anhydride was dissolved in 60 mL of dichloromethane, and the solution was slowly added to a round flask and stirred at room temperature for three hours. Once the reaction was complete, diethyl ether and a hydrochloride (HCl) aqueous solution were used for quenching and extraction. Then, a purification process was performed using column chromatography. Once the drying complete, 10.2 g of Intermediate 121001-i was obtained (yield: 71%).

LC-Mass (calculated value: 799.18 g/mol, found value: 800.01 (M+1))

### Synthesis of Intermediate 121001-j

2.64 g of Intermediate 121001-j was obtained in substantially the same manner as in Synthesis of Intermediate 112001-f in Synthesis Example 1, except that Intermediate 121001-i was used in reaction, followed by purification (yield: 50%).

LC-Mass (calculated value: 928.42 g/mol, found value: 927.97 (M+1))

### Synthesis of Compound 121001

1.65 g of Compound 121001 was obtained in substantially the same manner as in Synthesis of Intermediate 112001-d in Synthesis Example 1, except that Intermediate 121001-j was used in reaction (yield: 90%).

LC-Mass (calculated value: 848.4944 g/mol, found value: 848.4949 (M+1)) Synthesis Example 13: Synthesis of Compound 121002

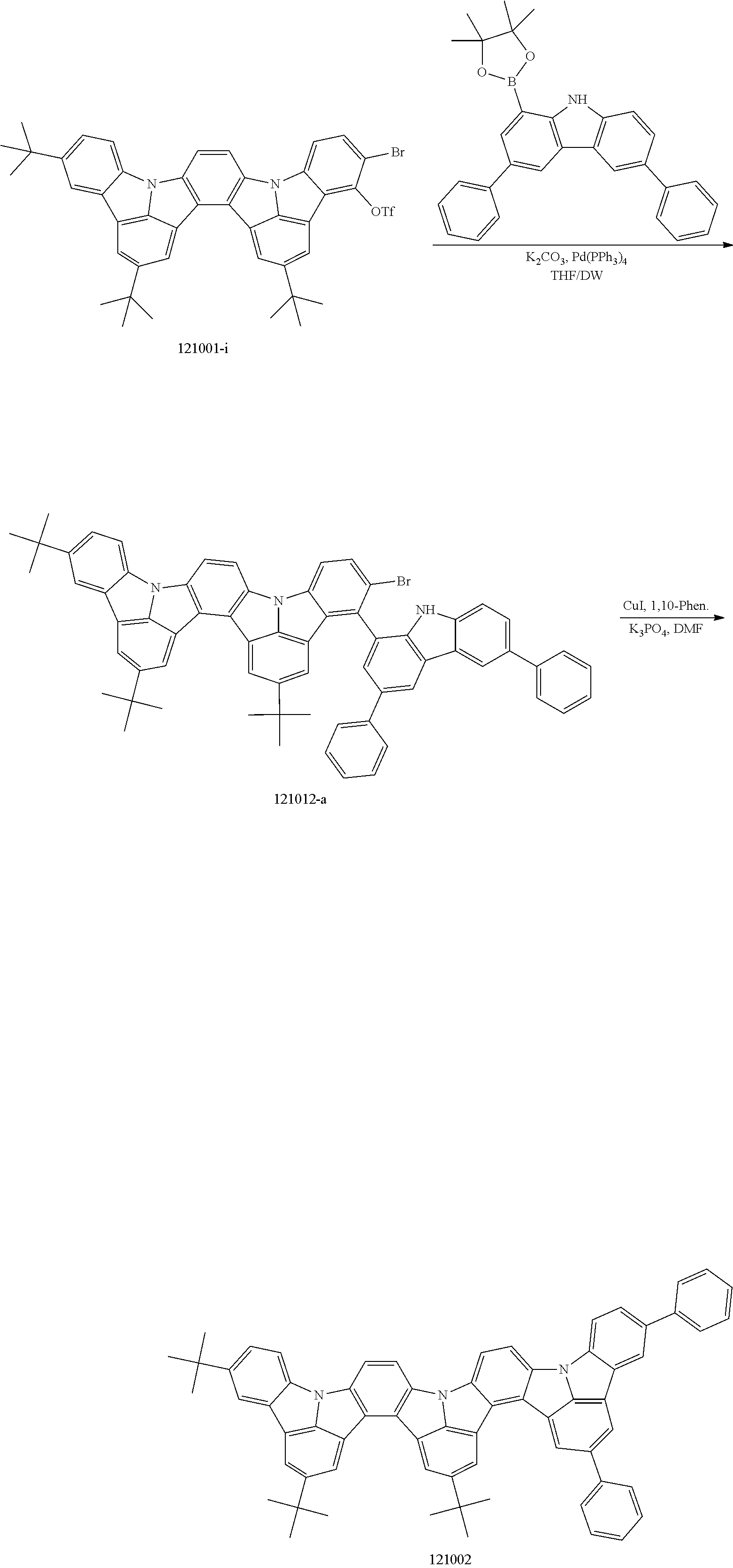 [SMILES: CC(C)(C)c1ccc2c(c1)c1cc(C(C)(C)C)cc3c4c5c6cc(C(C)(C)C)cc7c8c(OS(=O)(=O)C(F)(F)F)c(Br)ccc8n(c5ccc4n2c13)c76 ; CC1(C)OB(c2cc(-c3ccccc3)cc3c2[nH]c2ccc(-c4ccccc4)cc23)OC1(C)C ; CC(C)(C)c1ccc2c(c1)c1cc(C(C)(C)C)cc3c4c5c6cc(C(C)(C)C)cc7c8c(-c9cc(-c%10ccccc%10)cc%10c9[nH]c9ccc(-c%11ccccc%11)cc9%10)c(Br)ccc8n(c5ccc4n2c13)c76 ; CC(C)(C)c1ccc2c(c1)c1cc(C(C)(C)C)cc3c4c5c6cc(C(C)(C)C)cc7c8c9c%10cc(-c%11ccccc%11)cc%11c%12cc(-c%13ccccc%13)ccc%12n(c9ccc8n(c5ccc4n2c13)c76)c%11%10]

### Synthesis of Intermediate 121002-a

1.32 g of Intermediate 121001-a was obtained in substantially the same manner as in Synthesis of Intermediate 112001-f in Synthesis Example 1, except that Intermediate 121002-i was used in reaction (yield: 42%).

LC-Mass (calculated value: 968.36 g/mol, found value: 968.72 (M+1))

### Synthesis of Compound 121002

0.87 g of Compound 121002 was obtained in substantially the same manner as in Synthesis of Intermediate 112001-d in Synthesis Example 1, except that Intermediate 121002-a was used in reaction (yield: 88%).

LC-Mass (calculated value: 888.4318 g/mol, found value: 888.4317 (M+1))

### Synthesis Example 14: Synthesis of Compound 122001

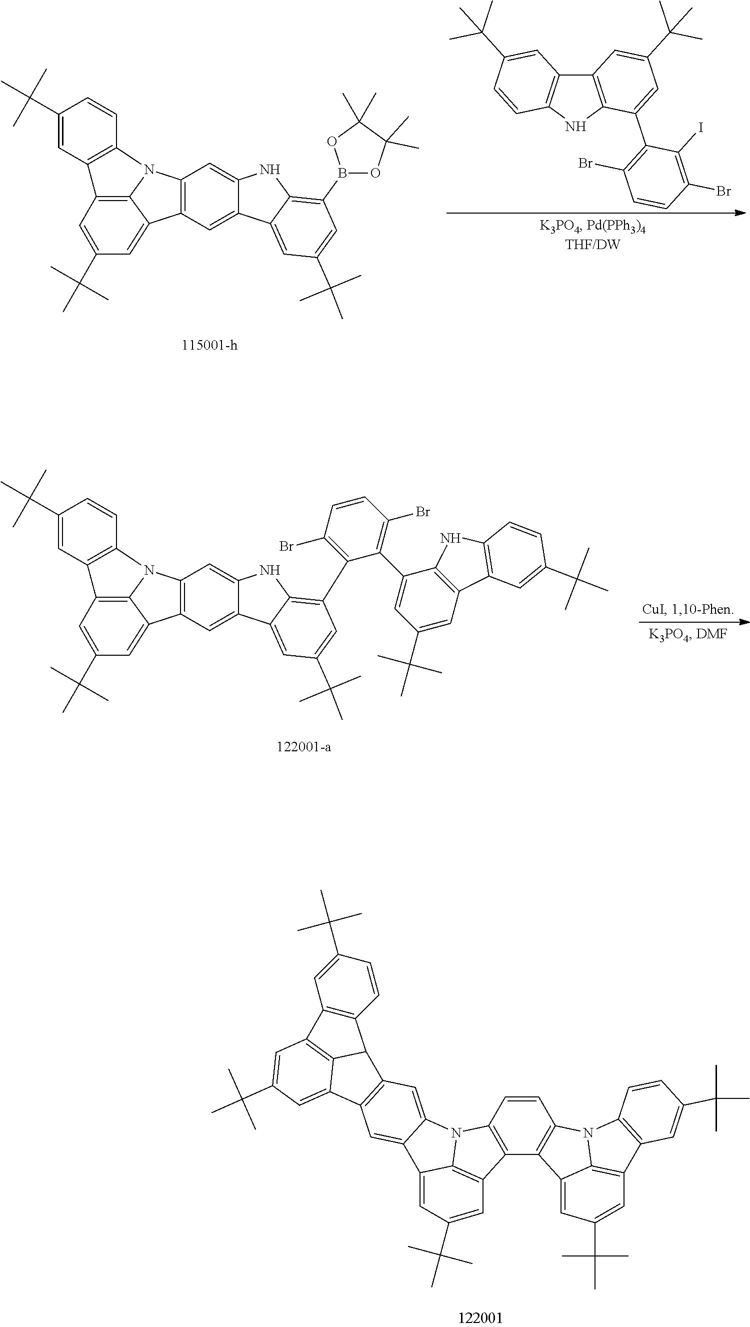

### Synthesis of Intermediate 122001-a

A reaction was performed in substantially the same manner as in Synthesis of Intermediate 112001-c in Synthesis Example 1, except that Intermediate 115001-h was used. 1.11 g of Intermediate 122001-a was obtained after purification completed (yield: 43%).

LC-Mass (calculated value: 1008.35 g/mol, found value: 1007.77 (M+1))

### Synthesis of Compound 122001

0.52 g of Compound 122001 was obtained in substantially the same manner as in Synthesis of Compound 112001 in Synthesis Example 1, except that Intermediate 122001-a was used in reaction (yield: 89%).

LC-Mass (calculated value: 848.4944 g/mol, found value: 848.4953 (M+1)) Synthesis Example 15: Synthesis of Compound 122002

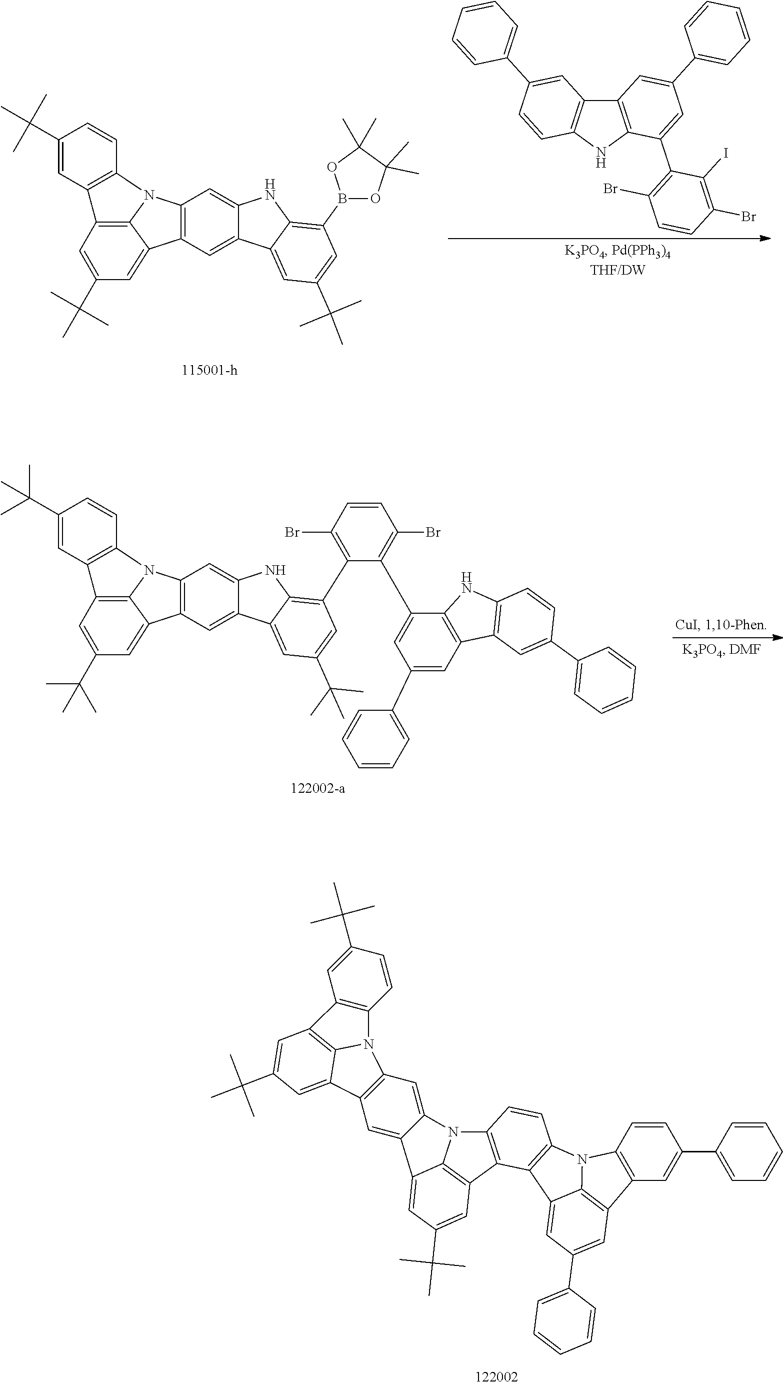

### Synthesis of Intermediate 122002-a

0.95 g of Intermediate 122002-a was obtained in substantially the same manner as in Synthesis of Intermediate 112001-c in Synthesis Example 1, except that Intermediate 115001-h was used in reaction (yield: 46%).

LC-Mass (calculated value: 1048.28 g/mol, found value: 1049.01 (M+1)) Synthesis of Compound 122002

0.532 g of Compound 122002 was obtained in substantially the same manner as in Synthesis of Compound 112001 in Synthesis Example 1, except that Intermediate 122002-a was used in reaction (yield: 79%).

LC-Mass (calculated value: 888.4318 g/mol, found value: 888.4320 (M+1))

### Synthesis Example 16: Synthesis of Compound 124001

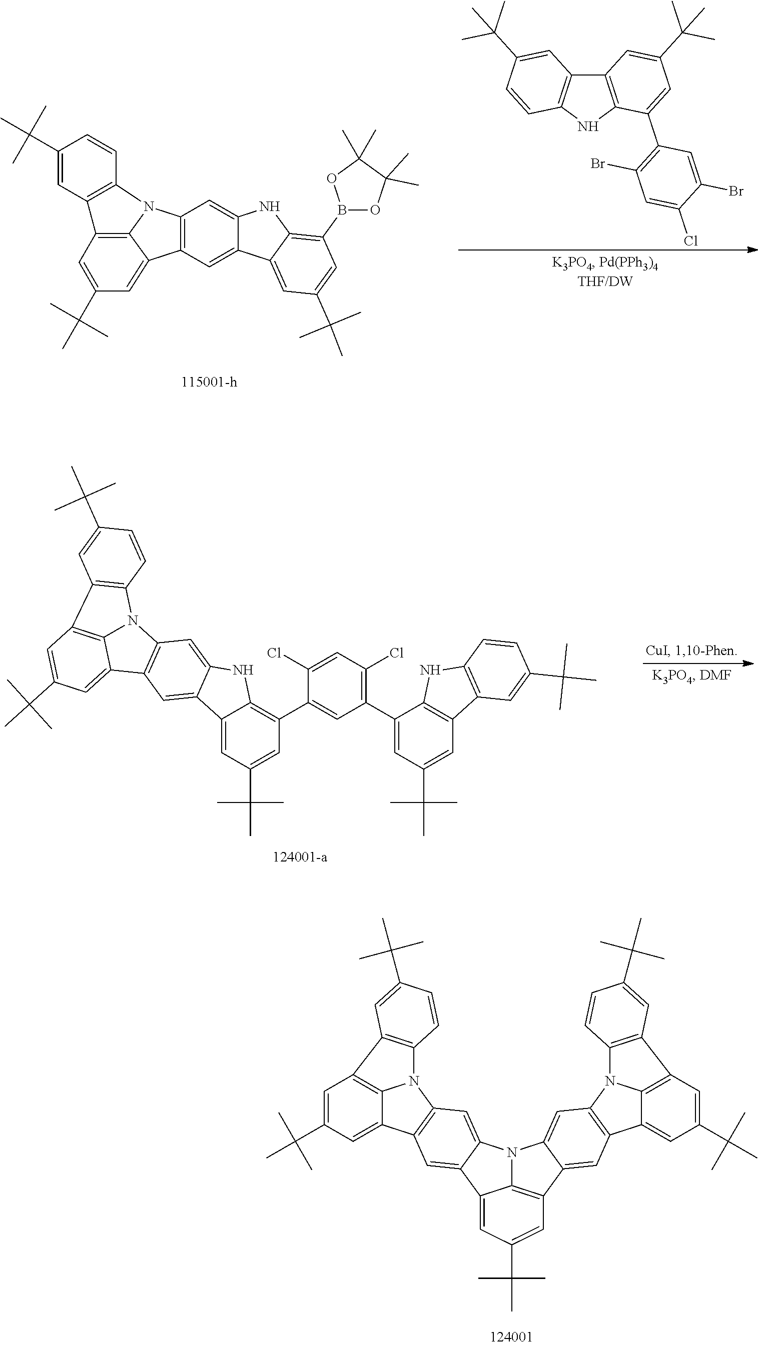

### Synthesis of Intermediate 124001-a

A reaction was performed in substantially the same manner as in Synthesis of Intermediate 112001-i in Synthesis Example 1, except that 1-(5-bromo-2,4-dichlorophenyl)-3,6-di-tert-butyl-9H-carbazole was used instead of 3,6-di-tert-butyl-1-(3,6-dibromo-2-iodophenyl)-9H-carbazole. Then, 1.23 g of Intermediate 124001-a was obtained (yield: 65%).

LC-Mass (calculated value: 920.45 g/mol, found value: 921.54 (M+1))

### Synthesis of Compound 124001

0.71 g of Compound 124001 was obtained in substantially the same manner as in Synthesis of Compound 112001 in Synthesis Example 1, except that dimethyl sulfoxide was used instead of DMF was used in reaction (yield: 64%).

LC-Mass (calculated value: 848.4944 g/mol, found value: 848.4946 (M+1))

### Synthesis Example 17: Synthesis of Compound 141001

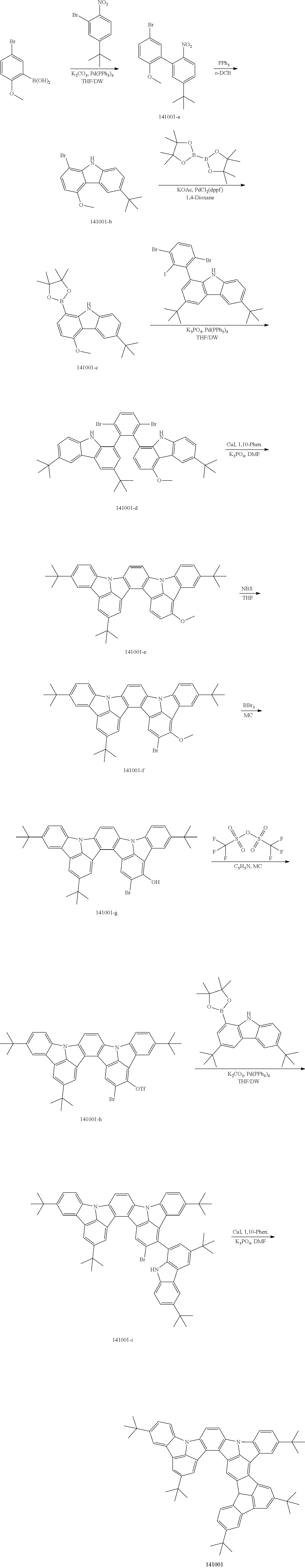

### Synthesis of Intermediate 141001-a

245 g of Intermediate 141001-a was obtained in substantially the same manner as in Synthesis of Intermediate 121001-b in Synthesis Example 12, except that 268 g (1.04 mol) of 2-bromo-4-(tert-butyl)-1-nitrobenzene was used instead of Intermediate 121001-a, followed by purification (yield: 78%).

LC-Mass (calculated value: 364.05 g/mol, found value: 364.72 (M+1))

### Synthesis of Intermediate 141001-b

100 g (0.275 mol) of Intermediate 141001-a, 216 g (0.824 mol) of triphenylphosphine (PPh₃), and 750 mL of 1,2-dichlorobenzne (o-DCB) were stirred together while heating at a temperature of 200° C. 5 hours later, 72.0 g (0.275 mol) of PPh₃ was added thereto to perform a reaction. Once the reaction was complete, excess o-DCB and PPh₃ were removed by using silica filter, and then, impurities were separated and purified through column chromatography. 73.9 g of Intermediate 141001-b was thus obtained (yield: 81%).

LC-Mass (calculated value: 332.07 g/mol, found value: 331.99 (M+1))

### Synthesis of Intermediate 141001-c

66.8 g of Intermediate 141001-c was obtained in substantially the same manner as in Synthesis of Intermediate 112001-h in Synthesis Example 1, except that 75.0 g (0.226 mmol) of Intermediate 141001-b was used instead of Intermediate 112001-g in reaction (yield: 78%).

LC-Mass (calculated value: 380.24 g/mol, found value: 381.01 (M+1))

### Synthesis of Intermediate 141001-d

38.3 g of Intermediate 141001-d was obtained in substantially the same manner as in Synthesis of Intermediate 112001-i in Synthesis Example 1, except that Intermediate 141001-c was used instead of Intermediate 112001-h (yield: 38%).

LC-Mass (calculated value: 763.19 g/mol, found value: 763.88 (M+1)) Synthesis of Intermediate 141001-e

A reaction was performed in substantially the same manner as in Synthesis of Compound 112001 in Synthesis Example 1, except that 42.0 g (54.9 mmol) of Intermediate 141001-d was used as a start material. 30.8 g of Intermediate 141001-e was thus obtained (yield: 93%).

LC-Mass (calculated value: 603.34 g/mol, found value: 603.97 (M+1))

### Synthesis of Intermediate 141001-f

14.2 g of Intermediate 141001-f was obtained in substantially the same manner as in Synthesis of Intermediate 121001-g in Synthesis Example 12, except that 20.0 g (33.2 mmol) of Intermediate 141001-e was used in reaction (yield: 63%).

LC-Mass (calculated value: 681.25 g/mol, found value: 682.01 (M+1))

### Synthesis of Intermediate 141001-g

9.99 g of Intermediate 141001-g was obtained in substantially the same manner as in Synthesis of Intermediate 121001-h in Synthesis Example 12, except that 15.0 g (22.0 mmol) of Intermediate 141001-f was used instead of Intermediate 121001-h in reaction (yield: 68%).

LC-Mass (calculated value: 667.23 g/mol, found value: 667.33 (M+1))

### Synthesis of Intermediate 141001-h

A reaction was performed n substantially the same manner as in Synthesis of Intermediate 121001-i in Synthesis Example 12, except that 5.00 g (7.49 mmol) of Intermediate 141001-g was used as a start material. 3.77 g of Intermediate 141001-h was thus obtained (yield: 63%).

LC-Mass (calculated value: 799.18 g/mol, found value: 800.01 (M+1))

### Synthesis of Intermediate 141001-i

1.05 g of Intermediate 141001-i was obtained in substantially the same manner as in Synthesis of Intermediate 121001-i in Synthesis Example 12, except that 2.00 g (2.50 mmol) of Intermediate 141001-h was used instead of Intermediate 121001-i in reaction (yield: 45%).

LC-Mass (calculated value: 928.42 g/mol, found value: 928.51 (M+1))

### Synthesis of Compound 141001

0.642 g of Compound 141001 was obtained in substantially the same manner as in Synthesis of Intermediate 112001-d in Synthesis Example 1, except that 0.800 g (0.861 mmol) of Intermediate 141001-i was in reaction (yield: 88%).

LC-Mass (calculated value: 848.4944 g/mol, found value: 848.4948 (M+1)) Synthesis Example 18: Synthesis of Compound 143001

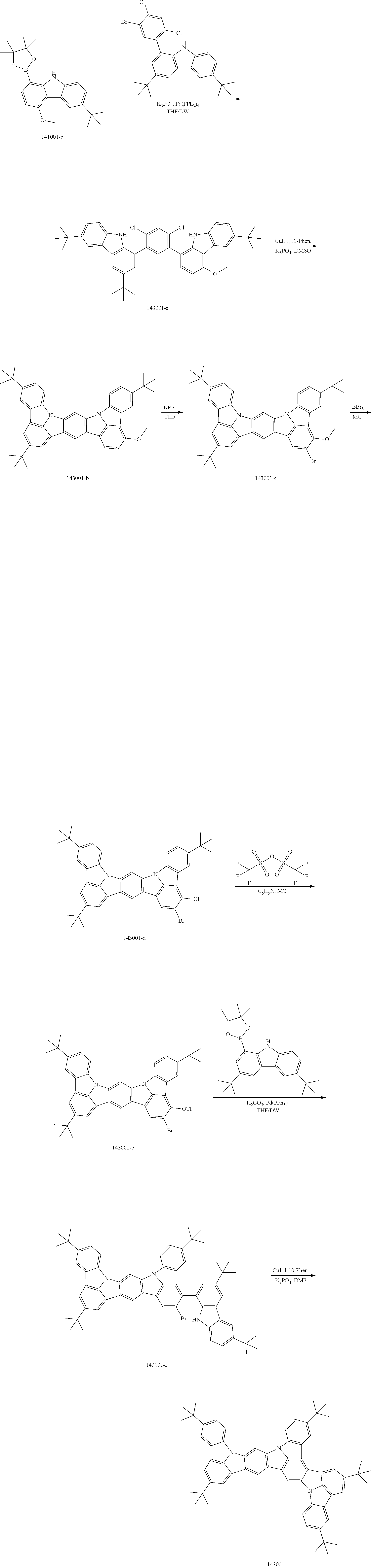

### Synthesis of Intermediate 143001-al

13.7 g of Intermediate 143001-a was obtained in substantially the same manner as in Synthesis of Intermediate 124001-a in Synthesis Example 2, except that Intermediate 141001-c was used in reaction (yield: 65%).

LC-Mass (calculated value: 675.29 g/mol, found value: 674.98 (M+1))

### Synthesis of Intermediate 143001-b

A reaction was performed in substantially the same manner as in Synthesis of Compound 124001 in Synthesis Example, except that Intermediate 143001-a was used. 10.2 g of Intermediate 143001-b was thus obtained (yield: 78%).

LC-Mass (calculated value: 603.34 g/mol, found value: 603.44 (M+1))

### Synthesis of Intermediate 143001-c

3.51 g of Intermediate 141001-c was obtained in substantially the same manner as in Synthesis of Intermediate 121001-g in Synthesis Example 12 (yield: 37%).

LC-Mass (calculated value: 681.25 g/mol, found value: 680.78 (M+1))

### Synthesis of Intermediate 143001-d

2.12 g of Intermediate 143001-d was obtained in substantially the same manner as in Synthesis of Intermediate 121001-h in Synthesis Example 12 (yield: 71%).

LC-Mass (calculated value: 667.23 g/mol, found value: 667.75 (M+1))

### Synthesis of Intermediate 143001-e

1.78 g of Intermediate 143001-e was obtained in substantially the same manner as in Synthesis of Intermediate 121001-i in Synthesis Example 12, except that Intermediate 143001-d was used in reaction (yield: 65%).

LC-Mass (calculated value: 799.18 g/mol, found value: 799.82 (M+1))

### Synthesis of Intermediate 143001-f

0.521 g of Intermediate 143001-f was obtained in substantially the same manner as in Synthesis of Intermediate 121001-j in Synthesis Example 12, except that Intermediate 143001-e was used in reaction (yield: 32%).

LC-Mass (calculated value: 928.42 g/mol, found value: 927.98 (M+1))

### Synthesis of Compound 143001

0.432 g of Compound 143001 was obtained in substantially the same manner as in Synthesis of Intermediate 112001-d in Synthesis Example 1 (yield: 89%).

LC-Mass (calculated value: 848.4944 g/mol, found value: 848.4944 (M+1)) Synthesis Example 19: Synthesis of Compound 153001

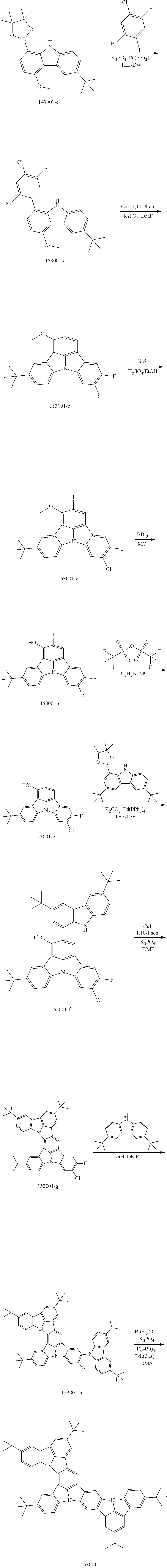

### Synthesis of Intermediate 153001-a

16.7 g of Intermediate 153001-a was obtained in substantially the same manner as in Synthesis of Intermediate 112001-c in Synthesis Example 1, except that Intermediate 141001-c and Intermediate 112001-b were used in reaction (yield: 42%).

LC-Mass (calculated value: 460.05 g/mol, found value: 461.01 (M+1))

### Synthesis of Intermediate 153001-b

10.2 g of Intermediate 153001-b was obtained in substantially the same manner as in Synthesis of Intermediate 112001-d in Synthesis Example 1, except that Intermediate 153001-a was used in reaction (yield: 91%).

LC-Mass (calculated value: 380.12 g/mol, found value: 380.55 (M+1))

### Synthesis of Intermediate 153001-c

25.0 g (65.8 mmol) of 153001-b and 400 mL of ethanol were added to a round flask and stirred. 65.8 mmol of sulfuric acid (H₂SO₄) was slowly added thereto, and 16.3 g (72.4 mmol) of N-iodosuccinimide (NIS) was dissolved in ethanol. The solution was slowly added to the reaction product. After stirring for 6 hours at room temperature, sodium hydrogen carbonate (NaHCO₃) and sodium thiosulfate (Na₂S₂O₃) were used for neutralization and quenching. An extraction process was performed using dichloromethane and distilled water. Then, impurities were separated and purified using column chromatography to thereby obtain 23.6 g of Intermediate 153001-c (yield: 71%).

LC-Mass (calculated value: 506.02 g/mol, found value: 506.44 (M+1))

### Synthesis of Intermediate 153001-d

11.7 g of Intermediate 153001-d was obtained in substantially the same manner as in Synthesis of Intermediate 121001-h in Synthesis Example 12 (yield: 55%).

LC-Mass (calculated value: 492.00 g/mol, found value: 491.88 (M+1))

### Synthesis of Intermediate 153001-e

12.1 g of Intermediate 153001-e was obtained in substantially the same manner as in Synthesis of Intermediate 121001-i in Synthesis Example 12, except that Intermediate 153001-d was used in reaction (yield: 66%).

LC-Mass (calculated value: 623.95 g/mol, found value: 624.11 (M+1)) Synthesis of Intermediate 153001-f

5.31 g of Intermediate 153001-f was obtained in substantially the same manner as in Synthesis of Intermediate 112001-i in Synthesis Example 1, except that Intermediate 153001-e was used (yield: 41%).

LC-Mass (calculated value: 775.24 g/mol, found value: 775.98 (M+1))

### Synthesis of Intermediate 153001-g

2.21 g of Intermediate 153001-g was obtained in substantially the same manner as in Synthesis of Intermediate 112001-d in Synthesis Example 1, except that Intermediate 153001-f was used in reaction (yield: 65%).

LC-Mass (calculated value: 625.28 g/mol, found value: 625.88 (M+1))

### Synthesis of Intermediate 153001-h

0.192 g (8.00 mmol) of sodium hydride (NaH) and 10 mL of DMF were added to a round flask and stirred. Then, a solution in which 1.07 g (3.84 mmol) of 3,6-di-tert-butyl-9H-carbazole was dissolved in 20 mL of DMF was slowly added thereto and stirred until the solution became transparent. Subsequently, 2.00 g (3.20 mmol) of Intermediate 153001-g was added thereto, followed by heating at a temperature of 150° C. to allow a reaction to occur. Once the reaction was complete, excess distilled water was added thereto, and a solid obtained by filtration was dissolved in dichloromethane. Then, precipitation was performed by using methanol. This process was performed repeatedly for filtration. After drying, 2.21 g of Intermediate 153001-h was obtained (yield: 78%).

LC-Mass (calculated value: 884.47 g/mol, found value: 885.02 (M+1))

### Synthesis of Compound 153001

1.50 g (1.70 mmol) of Intermediate 153001-h, 0.193 g (0.848 mmol) of benzyltriethylammonium chloride (BnEt₃NCI), 1.80 g (8.48 mmol) of potassium phosphate, 0.170 mmol of tri-tert-butylphosphine (P(t-Bu)₃), 0.078 g (0.085 mmol) of tris(dibenzylideneacetone)dipalladium(0) (Pd₂(dba)₃), and 50 mL of N,N-dimethylacetamide (DMA) were stirred and heated at a temperature of 200° C. Once the reaction was complete, excess chloroform was dissolved therein by heating. Then, the resulting mixture was subjected to filtration under reduced pressure using a filter charged with silica. The filtrate was then concentrated and precipitated using methanol. 1.11 g of Compound 153001 was obtained after drying (yield: 77%).

LC-Mass (calculated value: 848.4944 g/mol, found value: 848.4941 (M+1)) Synthesis Example 20: Synthesis of Compound 164001

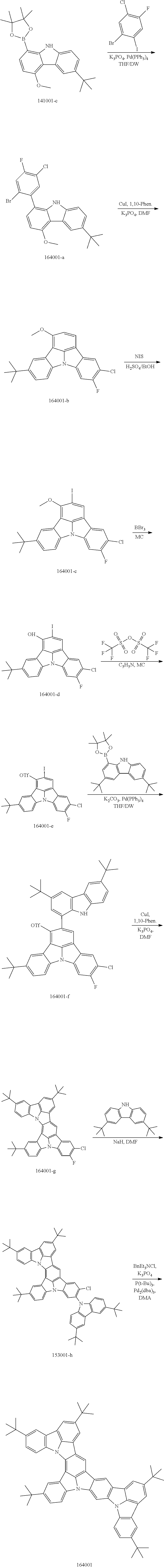

### Synthesis of Intermediate 164001-a

A reaction was performed in substantially the same manner as in Synthesis of Intermediate 153001-a in Synthesis Example 19, except that Intermediate 115001-b was used instead of Intermediate 112001-b. 20.8 g of Intermediate 164001-a was thus obtained (yield: 45%).

LC-Mass (calculated value: 460.05 g/mol, found value: 460.77 (M+1)) Synthesis of Intermediate 164001-b

A reaction was performed in substantially the same manner as in Synthesis of Intermediate 153001-b in Synthesis Example 19, except that Intermediate 164001-a was used instead of Intermediate 153001-a. 15.3 g of Intermediate 164001-b was thus obtained (yield: 88%).

LC-Mass (calculated value: 380.12 g/mol, found value: 380.41 (M+1)

### Synthesis of Intermediate 164001-c

A reaction was performed in substantially the same manner as in Synthesis of Intermediate 153001-c in Synthesis Example 19, except that Intermediate 164001-b was used instead of Intermediate 153001-b. 10.2 g of Intermediate 164001-c was thus obtained (yield: 66%).

LC-Mass (calculated value: 506.02 g/mol, found value: 506.14 (M+1))

### Synthesis of Intermediate 164001-d

A reaction was performed in substantially the same manner as in Synthesis of Intermediate 153001-d in Synthesis Example 19, except that Intermediate 164001-c was used instead of Intermediate 153001-c. 8.53 g of Intermediate 164001-d was thus obtained (yield: 71%).

LC-Mass (calculated value: 492.00 g/mol, found value: 491.95 (M+1))

### Synthesis of Intermediate 164001-e

A reaction was performed in substantially the same manner as in Synthesis of Intermediate 153001-e in Synthesis Example 19, except that Intermediate 164001-d was used instead of Intermediate 153001-d. Once the reaction and purification were complete, 5.42 g of Intermediate 164001-e was obtained (yield: 74%).

LC-Mass (calculated value: 623.95 g/mol, found value: 624.51 (M+1))

### Synthesis of Intermediate 164001-f

A reaction was performed in substantially the same manner as in Synthesis of Intermediate 153001-f in Synthesis Example 19, except that Intermediate 164001-e was used as a start material. 2.51 g of Intermediate 164001-f was thus obtained (yield: 32%).

LC-Mass (calculated value: 775.24 g/mol, found value: 775.57 (M+1))

### Synthesis of Intermediate 164001-g

A reaction was performed in substantially the same manner as in Synthesis of Intermediate 153001-g in Synthesis Example 19, except that Intermediate 164001-f was used instead of Intermediate 153001-f. 1.57 g of Intermediate 164001-g was thus obtained (yield: 75%).

LC-Mass (calculated value: 625.28 g/mol, found value: 625.93 (M+1)) Synthesis of Intermediate 164001-h

1.12 g of Intermediate 164001-h was obtained in substantially the same manner as in Synthesis of Intermediate 153001-h in Synthesis Example 19, except that Intermediate 164001-g was used in reaction (yield: 65%).

LC-Mass (calculated value: 884.47 g/mol, found value: 884.91 (M+1))

### Synthesis of Compound 164001

A reaction was performed in substantially the same manner as in Synthesis of Compound 153001 in Synthesis Example 19, except that Intermediate 164001-h was used in reaction. Once the synthesis was complete, 0.579 g of Compound 164001 was thus obtained (yield: 68%).

LC-Mass (calculated value: 848.4944 g/mol, found value: 848.4943 (M+1))

### Synthesis Example 21: Synthesis of Compound 174001

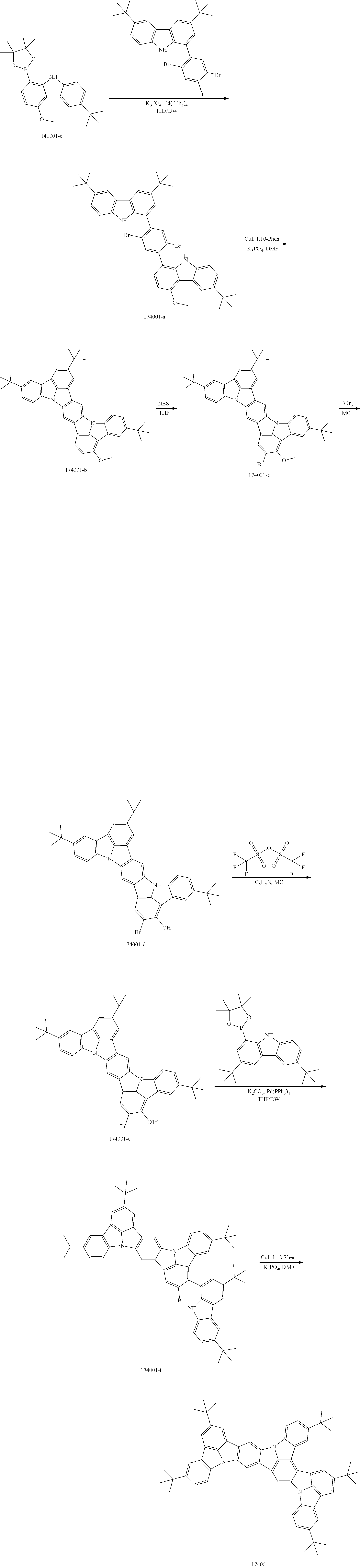

### Synthesis of Intermediate 174001-a

3.24 g of Intermediate 174001-a was obtained in substantially the same manner as in Synthesis of Intermediate 115001-i in Synthesis Example 3, except that Intermediate 141001-c and Intermediate 115001-h were used in reaction (yield: 42%).

LC-Mass (calculated value: 763.19 g/mol, found value: 762.87 (M+1))

### Synthesis of Intermediate 174001-b

2.89 g of Intermediate 174001-b was obtained in substantially the same manner as in Synthesis of Compound 112001 in Synthesis Example 1, except that Intermediate 174001-a was used in reaction (yield: 97%).

LC-Mass (calculated value: 603.34 g/mol, found value: 602.98 (M+1))

### Synthesis of Intermediate 174001-c

A reaction was performed in substantially the same manner as in Synthesis of Intermediate 121001-g in Synthesis Example 12, except that Intermediate 174001-b was used in reaction. 1.79 g of Intermediate 174001-c was obtained (yield: 75%).

LC-Mass (calculated value: 681.25 g/mol, found value: 681.82 (M+1))

### Synthesis of Intermediate 174001-d

1.42 g of Intermediate 174001-d was obtained in substantially the same manner as in Synthesis of Intermediate 121001-h in Synthesis Example 12, except that Intermediate 174001-c was used instead of Intermediate 121001-g (yield: 63%).

LC-Mass (calculated value: 667.23 g/mol, found value: 667.42 (M+1))

### Synthesis of Intermediate 174001-e

A reaction was performed in substantially the same manner as in Synthesis of Intermediate 121001-i in Synthesis Example 12, except that Intermediate 174001-d was used. 1.23 g of Intermediate 174001-e was obtained (yield: 58%).

LC-Mass (calculated value: 799.18 g/mol, found value: 800.21 (M+1))

### Synthesis of Intermediate 174001-f

0.852 g of Intermediate 174001-f was obtained in substantially the same manner as in Synthesis of Intermediate 112001-f in Synthesis Example 1, except that Intermediate 174001-e was used in reaction (yield: 59%).

LC-Mass (calculated value: 928.42 g/mol, found value: 928.66 (M+1))

### Synthesis of Compound 1

0.512 g of Compound 174001 was obtained in substantially the same manner as in Synthesis of Intermediate 112001-d in Synthesis Example 1, except that Intermediate 174001-f was used in reaction (yield: 92%).

LC-Mass (calculated value: 848.4944 g/mol, found value: 848.4944 (M+1)) Evaluation Example 1: Evaluation on HOMO, LUMO, T₁, and S₁ energy levels

The HOMO, LUMO, T₁ and S₁ energy levels of the compounds shown in Table 2 were measured according to the method described in Table 17. The results thereof are shown in Table 18:

TABLE 17

| | |
|---|---|
| HOMO | A potential (Volts, V) versus current (Amperes, A) graph of |
| energy | each compound was obtained by using cyclic voltammetry |
| level | (CV) (electrolyte: 0.1M TBAP/solvent: ACN/electrode: |
| evaluation | 3-electrode system (working electrode: carbon, |
| method | reference electrode: Ag/AgCl, auxiliary electrode: Pt)). |
| | Subsequently, from oxidation onset of the graph, |
| | a HOMO energy level of the compound was calculated. |
| LUMO | Each compound was diluted at a concentration of |
| energy | 1 × 10⁻⁵M in tetrahydrofuran, and |
| level | an UV absorption spectrum thereof was measured at room |
| evaluation | temperature by using JASCO V-730 UV-visible |
| method | spectrometer. A LUMO energy level thereof was |
| | calculated by using an optical band gap (E_{g}) from |
| | an edge of the absorption spectrum and a HOMO |
| | energy level. |
| T₁ | Each compound was dissolved at a concentration of |
| energy | 1 × 10⁻⁵M in tetrahydrofuran and |
| level | placed in a quartz cell, followed by cooling by using |
| evaluation | nitrogen (77 Kelvins (K)). Then, 1 millisecond |
| method | (ms) of delay time was given by using a fluorescence |
| | spectrometer (PerkinElmer, LS-55) to measure a |
| | photoluminescence spectrum. |
| S₁ | S₁ energy level was measured in |
| energy | substantially the same manner as in T₁ energy |
| level | level evaluation method, except that delay time |
| evaluation | was not given, and the maximum emission peak was |
| method | determined as S₁ energy level. |

TABLE 18

| Compound | HOMO | LUMO | T₁ | S₁ | ΔE_{ST} |
|---|---|---|---|---|---|
| No. | (eV) | (eV) | (eV) | (eV) | (eV) |
| 115001 | −5.63 | −2.85 | 2.54 | 2.84 | 0.30 |
| 115002 | −5.63 | −2.89 | 2.54 | 2.80 | 0.26 |
| C1 | −5.72 | −2.93 | 2.55 | 2.84 | 0.29 |
| C2 | −5.89 | −3.15 | 2.54 | 2.80 | 0.26 |
| C3 | −5.53 | −2.88 | 2.70 | 2.49 | 0.21 |

Referring to the results of Table 18, Compounds 115001 and 115002 were each found to have improved electrical characteristics. Evaluation Example 2: Evaluation of full width at half maximum (FWHM)

As the method described in Table 19, photoluminescence spectra of the compounds shown in Table 20 were measured, and the FWHM of each compound was evaluated. The results thereof are shown in Table 20.

TABLE 19

| | |
|---|---|
| Measurement | Each compound and mCP and TSPO1 were co- |
| of photo- | deposited on a quartz substrate at a weight ratio of |
| luminescence | 50:50 (wherein, the weight of the evaluated |
| (PL) | compound of the total weight of the host and each |
| spectrum | evaluated compound was 1 wt %) to form a thin |
| | film having a thickness of 40 nm. Then, the |
| | spectrum thereof was measured at room |
| | temperature by using a fluorescence spectrometer |
| | (PerkinElmer, LS-55). |

TABLE 20

| Compound No. | FWHM (nm) |
|---|---|
| 115001 | 21 |
| 115002 | 23 |
| C1 | 22 |
| C2 | 21 |
| C3 | 28 |

Referring to the results of Table 20, Compounds 115001 and 115002 were each found to have improved color characteristics. Evaluation Example 3: Evaluation of photoluminescent quantum yield (PLQY) and decay time

### (1) Preparation of Thin Film

A quartz substrate was prepared by washing with chloroform and pure water. Then, as shown in Table 21, each compound and Host Compound mCP and TSPO1 were co-deposited at a weight ratio of 5:5 (wherein, the weight of each evaluated compound of the total weight of the host and each evaluated compound was 1 wt %) at a vacuum pressure of 10⁻⁷ torr to thereby form a thin film having a thickness of 40 nm. (Co-deposition was performed in the same manner as described in Table 19.)

### (2) Evaluation of Photoluminescent Quantum Yield

Photoluminescent quantum yields in the thin film was evaluated by using Hamamatsu Photonics absolute PL quantum yield measurement system employing PLQY measurement software (Hamamatsu Photonics, Ltd., Shizuoka, Japan), in which a xenon light source, a monochromator, a photonic multichannel analyzer, and an integrating sphere are mounted. Thus, PLQY of the compounds shown in Table 21 were measured accordingly.

### (3) Decay Time Evaluation

TRPL measurement system C11367-31 (Hamamatsu Photonics) and a mounted LED lamp (excitation wavelength of 280 nanometers) were used to evaluate the PL spectrum of each thin film under nitrogen atmosphere at room temperature to thereby determine the maximum emission peak. A repetition rate of excitation on the thin film by the LED lamp was set to 1 kilohertz (KHz), and the number of photons emitted from the wavelength of the maximum emission peak was counted repeatedly to thereby obtain a TRPL curve that may be analyzed. T_{decay}(Ex) (decay time) of the thin film was obtained by fitting at least two exponential decay functions to the results thereof. The functions used for the fitting are as described in Equation 1, and a decay time T_{decay} having the largest value among values for each of the exponential decay functions used for the fitting was taken as T_{decay}(Ex), i.e., a decay time. The results thereof are shown in Table 21. The remaining decay time T_{decay} values were used to determine the lifetime of typical fluorescence to be decayed. Here, during the same measurement time as the measurement time for obtaining TRPL curves, the same measurement was repeated once more in a dark state (i.e., a state where a pumping signal incident on each of the films was blocked), thereby obtaining a baseline or a background signal curve available as a baseline for the fitting:

$$f ( t ) = {\sum }_{i = 1}^{n} {A}_{i} exp ( - t / {T}_{decay , i} ) \qquad Equation 1$$

TABLE 21

| | | T_{decay} (Ex) (μs) |
|---|---|---|
| Compound No. | PLQY | (decay time) |
| 115001 | 99 | 4060 |
| 115002 | 97 | 1760 |
| C1 | 95 | 12500 |
| C2 | 98 | 1460 |
| C3 | 97 | 183 |

Referring to the results of Table 21, Compounds 115001 and 115002 were each found to have improved PLQY thin film characteristics and excellent device characteristics, although Compounds 115001 and 115002 had a relatively long decay time.

### Example 1-1

An ITO glass substrate was cut to a size of 50 millimeters (mm)×50 mm×0.5 mm. Then the glass substrate was sonicated in acetone isopropyl alcohol and pure water for about 15 minutes each, and cleaned by exposure to ultraviolet rays and ozone for 30 minutes.

Subsequently, PEDOT:PSS was spin-coated on an ITO electrode (anode) on a glass substrate to form a first hole injection layer having a thickness of 40 nm. TAPC was then deposited on the first hole injection layer to form a second hole injection layer having a thickness of 5 nm. TCTA was deposited on the second hole injection layer to form a first hole transport layer having a thickness of 5 nm. PCzAc was deposited on the first hole transport layer to form a second hole transport layer having a thickness of 5 nm. Then, mCP was deposited on the second hole transport layer to form an electron blocking layer having a thickness of 5 nm. Thus, a hole transport region was formed.

mCP (as a first host), TSPO1 (as a second host), and Compound 115001 (as a dopant) were co-deposited on the hole transport region (wherein, the weight of the dopant of the total weight of the first host, the second host, and the dopant was 1 wt %) to thereby form an emission layer having a thickness of 25 nm.

Thereafter, TSPO1 was deposited on the emission layer to form an electron transport layer having a thickness of 25 nm, LiF was deposited on the electron transport layer to form an electron injection layer having a thickness of 1.5 nm, and AI was deposited on the electron injection layer to a thickness of 200 nm, thereby completing the manufacture of an organic light-emitting device.

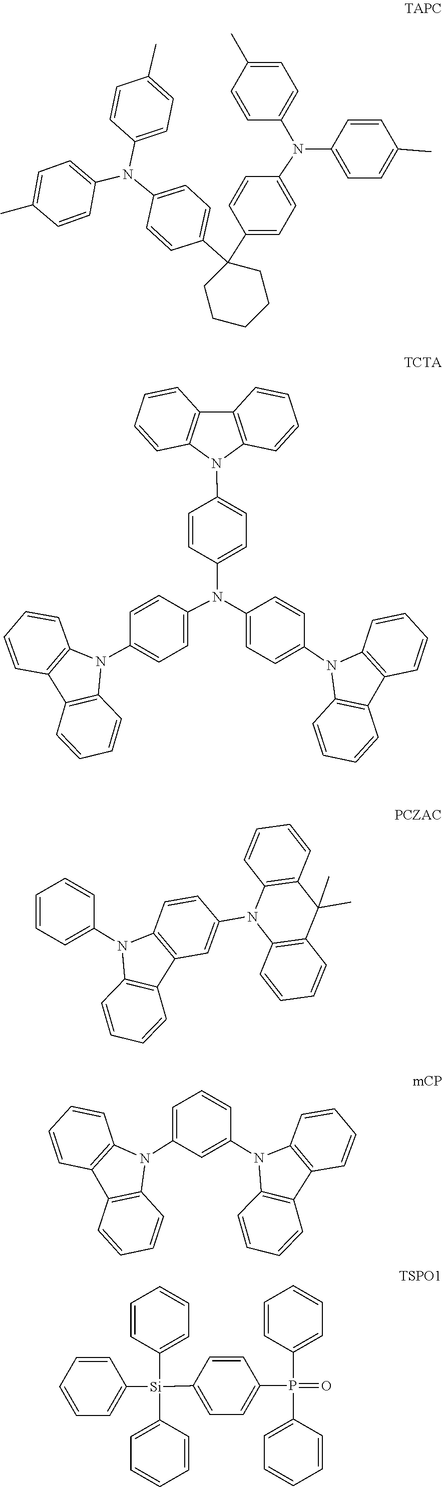

### Example 1-2 and Comparative Examples 1-1 to 1-3

Organic light-emitting devices were manufactured in substantially the same manner as in Example 1-1, except that compounds shown in Table 22 were used as a dopant in the formation of an emission layer.

### Example 2-1

An ITO glass substrate was cut to a size of 50 millimeters (mm)×50 mm×0.5 mm. Then the glass substrate was sonicated in acetone isopropyl alcohol and pure water for about 15 minutes each, and cleaned by exposure to ultraviolet rays and ozone for 30 minutes.

Subsequently, PEDOT:PSS was spin-coated on an ITO electrode (anode) on a glass substrate to form a first hole injection layer having a thickness of 40 nm. BDCFL was then deposited on the first hole injection layer to form a second hole injection layer having a thickness of 10 nm. TNPA was deposited on the second hole injection layer to form a first hole transport layer having a thickness of 10 nm. PCzAc was deposited on the first hole transport layer to form a second hole transport layer having a thickness of 5 nm. Then, mCP was deposited on the second hole transport layer to form an electron blocking layer having a thickness of 5 nm. Thus, a hole transport region was formed.

Compound Host* and Compound 115001 (as a dopant) were co-deposited on the hole transport region (wherein, the total weight of the host and the dopant was 1 wt %) to thereby form an emission layer having a thickness of 30 nm.

TNPT was deposited on the emission layer to form a hole blocking layer having a thickness of 5 nm, and ZADN was deposited on the hole blocking layer to form an electron transport layer having a thickness of 20 nm. Next, LiF was deposited on the electron transport layer to form an electron injection layer having a thickness of 1.5 nm, and aluminum (Al) was then deposited on the electron injection layer to a thickness of 200 nm, thereby completing the manufacture of an organic light-emitting device.

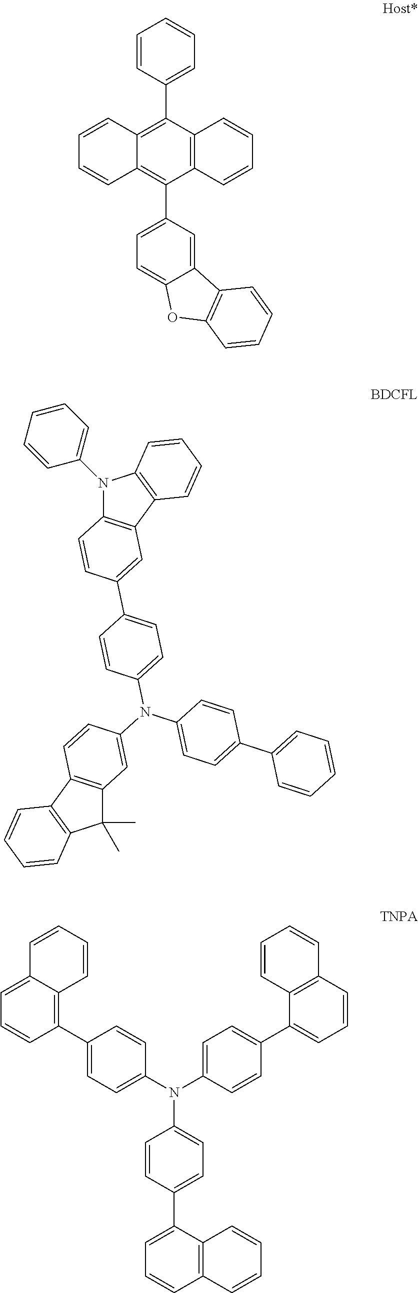 [SMILES: c1ccc(-c2c3ccccc3c(-c3ccc4oc5ccccc5c4c3)c3ccccc23)cc1 ; CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc2c(-c3ccc(N(c4ccc(-c5cccc6ccccc56)cc4)c4ccc(-c5cccc6ccccc56)cc4)cc3)cccc2c1]

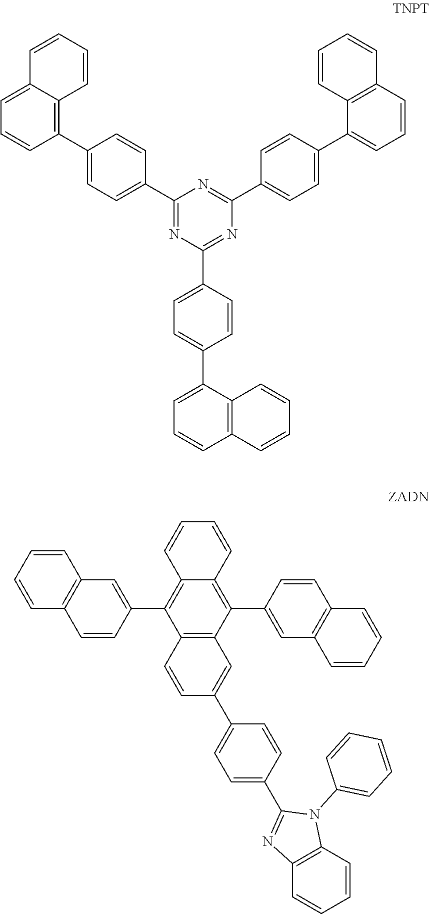 [SMILES: c1ccc2c(-c3ccc(-c4nc(-c5ccc(-c6cccc7ccccc67)cc5)nc(-c5ccc(-c6cccc7ccccc67)cc5)n4)cc3)cccc2c1 ; c1ccc(-n2c(-c3ccc(-c4ccc5c(-c6ccc7ccccc7c6)c6ccccc6c(-c6ccc7ccccc7c6)c5c4)cc3)nc3ccccc32)cc1]

### Examples 2-2 and Comparative Examples 2-1 to 2-3

Organic light-emitting devices were manufactured in substantially the same manner as in Example 2-1, except that compounds shown in Table 23 were used as a dopant in the formation of an emission layer.

### Example 3-1

An ITO glass substrate was cut to a size of 50 millimeters (mm)×50 mm×0.5 mm. Then the glass substrate was sonicated in acetone isopropyl alcohol and pure water for about 15 minutes each, and cleaned by exposure to ultraviolet rays and ozone for 30 minutes.

Subsequently, PEDOT:PSS was spin-coated on an ITO electrode (anode) on a glass substrate to form a hole injection layer having a thickness of 90 nm. TAPC was then deposited on the hole injection layer to form a hole transport layer having a thickness of 20 nm. mCP was deposited on the hole transport layer to form an electron blocking layer having a thickness of 10 nm. Thus, a hole transport region was formed.

DPEPO (as a host), DMAC-DPS (as a sensitizer), and Compound 115001 (as a dopant) (wherein, the weight of the dopant of the total weight of the host, the sensitizer, and the dopant was 1 wt %, and the weight of the sensitizer of the total weight of the host, the sensitizer, and the dopant was 20 wt %) were co-deposited on the hole transport region to form an emission layer having a thickness of 25 nm.

Next, TSPO1 was deposited on the emission layer to form a first electron transport layer having a thickness of 5 nm. Then, TPBi was deposited on the first electron transport layer to form a second electron transport layer having a thickness of 20 nm. LiF was then deposited on the second electron transport layer to form an electron injection layer having a thickness of 1.5 nm. Finally, aluminum (Al) was deposited on the electron injection layer to a thickness of 200 nm, thereby completing the manufacture of a light-emitting device.

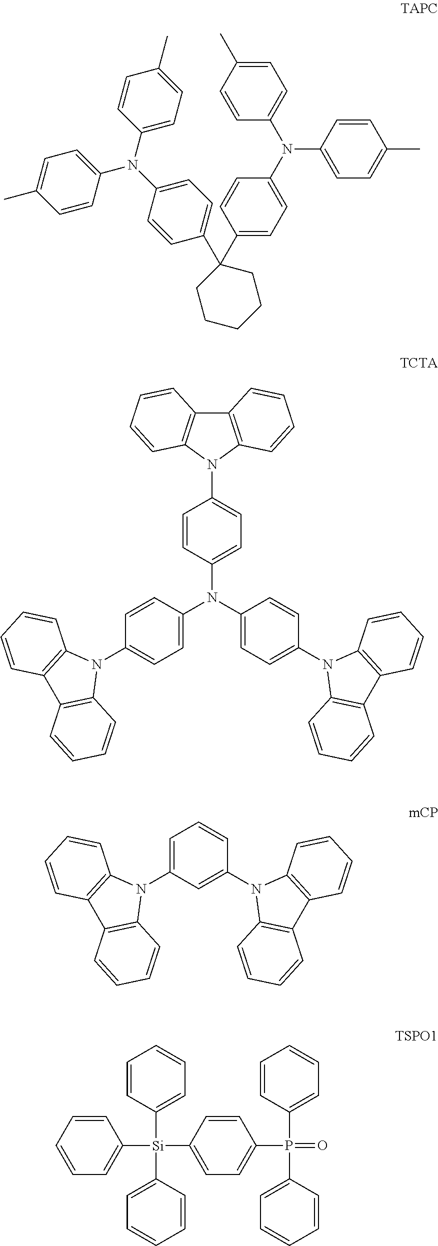

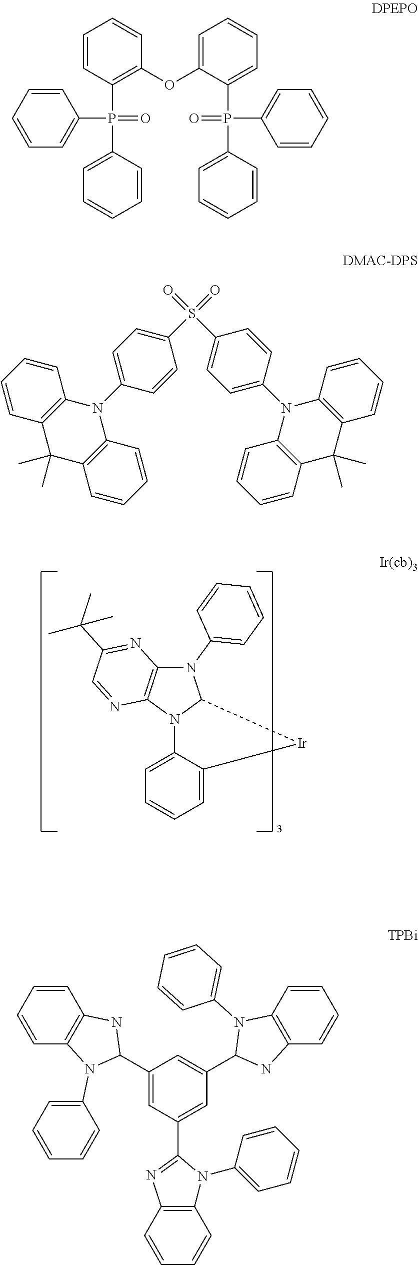

### Example 3-2 and Comparative Example 3-1

Organic light-emitting devices were manufactured in substantially the same manner as in Example 3-1, except that compounds shown in Table 24 were used as a sensitizer and a dopant in the formation of an emission layer.

### Evaluation Example: Evaluation OLED characteristics

The driving voltage, maximum external quantum yield, electricity efficiency, current efficiency, ClE color-coordinate (at 1,000 nit), conversion efficiency, and FWHM of the organic light-emitting devices of the Examples and the Comparative Examples were measured by using a current voltmeter (Keithley 2400) and a luminance meter (Minolta Cs-1000A). The results thereof are shown in Tables 22 to 24. Here, Comparative Examples C₁ and 02 were not operated and thus not be evaluated.

TABLE 22

| | | | Driving | | Electricity | Current | | Conversion |
|---|---|---|---|---|---|---|---|---|
| | | FWHM | voltage | EQEₘₐₓ | efficiency | efficiency | | efficiency |
| | Dopant | (nm) | (V) | (%) | (%) | (%) | CIEy | (%) |
| Example 1-1 | 115001 | 11 | 5.7 | 180 | 141 | 133 | 0.044 | 184 |
| Example 1-2 | 115002 | 15 | 5.7 | 179 | 175 | 175 | 0.061 | 175 |
| Comparative | C1 | 22 | 5.7 | 100 | 100 | 100 | 0.061 | 100 |
| Example 1-1 | | | | | | | | |
| Comparative | C2 | 21 | 5.4 | 124 | 126 | 134 | 0.068 | 120 |
| Example 1-2 | | | | | | | | |
| Comparative | C3 | 21 | 5.1 | 193 | 373 | 351 | 0.171 | 125 |
| Example 1-3 | | | | | | | | |

TABLE 23

| | | | Driving | | Electricity | Current | | Conversion |
|---|---|---|---|---|---|---|---|---|
| | | FWHM | voltage | EQEₘₐₓ | efficiency | efficiency | | efficiency |
| | Dopant | (nm) | (V) | (%) | (%) | (%) | CIEy | (%) |
| Example 2-1 | 115001 | 24 | 3.9 | 138 | 234 | 144 | 0.052 | 194 |
| Example 2-2 | 115002 | 25 | 3.8 | 135 | 206 | 127 | 0.068 | 130 |
| Comparative | C1 | 40 | 5.9 | 100 | 100 | 100 | 0.070 | 100 |
| Example 2-1 | | | | | | | | |
| Comparative | C2 | 34 | 5.7 | 111 | 143 | 111 | 0.076 | 102 |
| Example 2-2 | | | | | | | | |
| Comparative | C3 | 34 | 5.2 | 133 | 294 | 220 | 0.179 | 86 |
| Example 2-3 | | | | | | | | |

TABLE 24

| | | | Driving | | Electricity | Current | | Conversion |
|---|---|---|---|---|---|---|---|---|
| | | | voltage | EQEₘₐₓ | efficiency | efficiency | | efficiency |
| | Sensitizer | Dopant | (V) | (%) | (lm/W) | (Cd/A) | CIEy | (Cd/A/y) |
| Example 3-1 | DMAC- | 115001 | 6.5 | 108 | 112 | 112 | 0.173 | 106 |
| | DPS | | | | | | | |
| Example 3-2 | Ir(cb)₃ | 115001 | 5.9 | 89 | 98 | 98 | 0.192 | 84 |
| Comparative | Ir(cb)₃ | C3 | 5.8 | 100 | 100 | 100 | 0.165 | 100 |
| Example 3-1 | | | | | | | | |

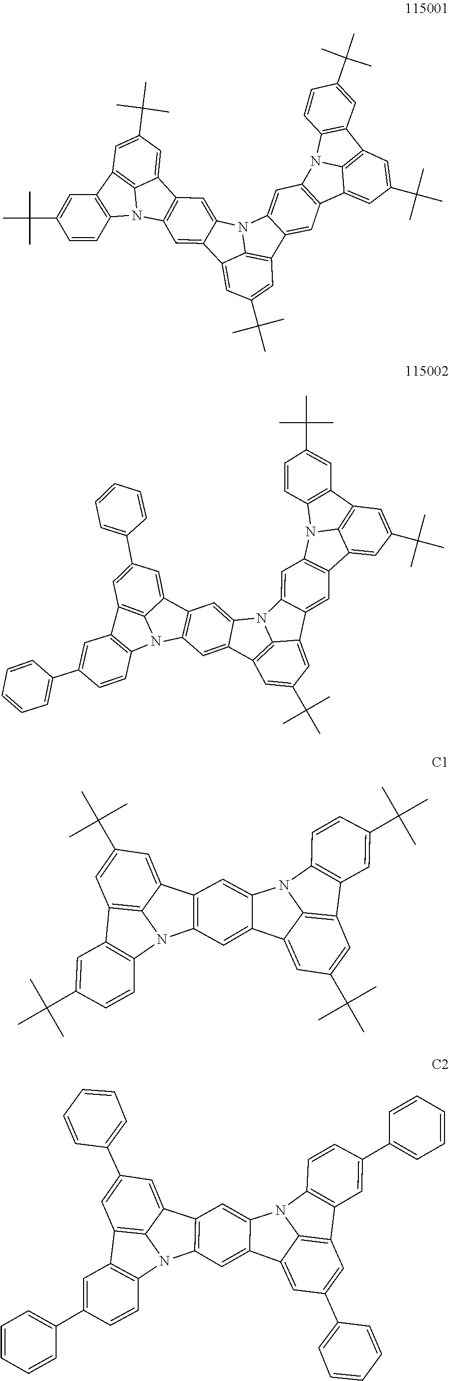

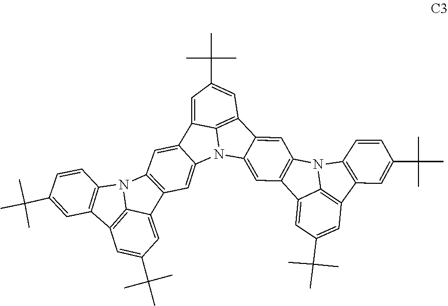 [SMILES: CC(C)(C)c1ccc2c(c1)c1cc(C(C)(C)C)cc3c4cc5c(cc4n2c13)c1cc(C(C)(C)C)cc2c3cc4c(cc3n5c12)c1cc(C(C)(C)C)cc2c3cc(C(C)(C)C)ccc3n4c21]

Referring to the results of Tables 22 to 24, the organic light-emitting devices of the Examples were found to have high colorimetric purity and/or high efficiency, as compared with the organic light-emitting device of the Comparative Examples. In particular, Comparative Example 1-3 was found to have a relatively great FWHM and a significantly deteriorated conversion efficiency, i.e., efficiency at a desired CIEy, as compared with Examples 1-1 and 1-2. That is, even when 3 ICz(s) are included, a specific geometrical structure needs to be satisfied to provide an organic light-emitting device having high colorimetric purity and/or high efficiency.

In addition, referring to the results of Tables 22 to 24, the heterocyclic compound was found to be applicable to organic light-emitting devices that emit according to various mechanisms. On the other hand, Compounds C₁ to C₃ did not emit in devices having a similar structure as Example 3-1. Thus, comparative devices that correspond to Example 3-1 were not be manufactured by using Compounds C₁ to C₃. Likewise, Compounds C₁ and C₂ did not emit in devices having a similar structure as Example 3-2. Thus, comparative devices that correspond to Example 3-2 were not be manufactured by using Compounds C₁ and C₂.

As apparent from the foregoing description, an organic light-emitting device including the heterocyclic compound may have improved efficiency and/or colorimetric purity.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope as defined by the following claims.

What is claimed is:

1. A heterocyclic compound represented by one of Formulae 1-1 and 1-2: 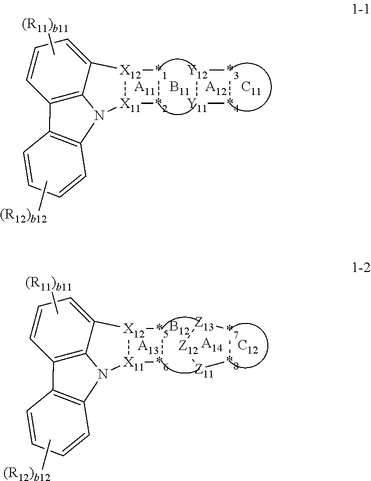
wherein, in Formulae 1-1 and 1-2, A₁₁ and A₁₃ are each independently a group represented by Formula 2-1, A₁₂ is a group represented by Formula 2-2, and A₁₄ is a group represented by Formula 2-3: 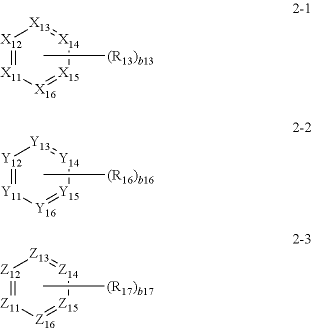
wherein, in Formula 2-1 to 2-3, X₁₁ to X₁₆, Y₁₁ to Y₁₆, and Z₁₁ to Z₁₆ are each independently a carbon atom, any suitable two adjacent groups X₁₃ to X₁₆ are *1 and *2 in Formula 1-1 or *5 and *6 in Formula 1-2, and any suitable two adjacent groups Y₁₃ to Y₁₆ are *3 and *4 in Formula 1-1, and any suitable two adjacent groups Z₁₄ to Z₁₆ are *7 and *8 in Formula 1-2,
B₁₁ is a group represented by Formula 2-4: 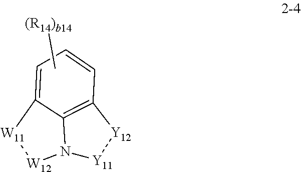
wherein, in Formula 2-4, W₁₁ and W₁₂ are each independently a carbon atom, and W₁₁ and W₁₂ are respectively *1 and *2 in Formula 1-1, B₁₂ is a group represented by Formula 2-5: 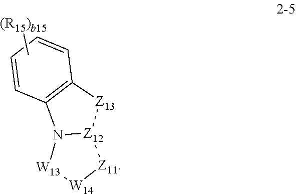
In Formula 2-5, W₁₃ and W₁₄ are each independently a carbon atom, and W₁₃ and W₁₄ are respectively *5 and *6 in Formula 1-2,
C₁₁ and C₁₂ are each independently a group represented by Formula 2-6: 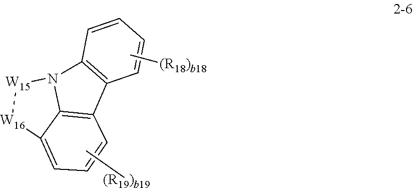
wherein, in Formula 2-6, W₁₅ and W₁₆ are each independently a carbon atom, and W₁₅ and W₁₆ are respectively *3 and *4 in Formula 1-1 or *7 and *8 in Formula 1-2,
R₁₁ to R₁₉ are each independently hydrogen, deuterium, —F, —Cl, —Br, —I, —SF₅, a hydroxyl group, a cyano group, a nitro group, an amidino group, a hydrazino group, a hydrazono group, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C—C₆₀ aryl group, a substituted or unsubstituted C₇-C₆₀ alkyl aryl group, a substituted or unsubstituted C₆-C₆₀ aryloxy group, a substituted or unsubstituted C—C₆₀ arylthio group, a substituted or unsubstituted C₁-C₆₀ heteroaryl group, a substituted or unsubstituted C₂-C₆₀ alkyl heteroaryl group, a substituted or unsubstituted C₁-C₆₀ heteroaryloxy group, a substituted or unsubstituted C₁-C₆₀ heteroarylthio group, a substituted or unsubstituted monovalent aromatic condensed polycyclic group, a substituted or unsubstituted monovalent aromatic condensed heteropolycyclic group, a substituted or unsubstituted monovalent non-aromatic condensed polycyclic group, a substituted or unsubstituted monovalent non-aromatic condensed heteropolycyclic group, —Si(Q₁)(Q₂)(Q₃), —Ge(Q₁)(Q₂)(Q₃), —C(Q₁)(Q₂)(Q₃), —B(Q₁)(Q₂), —N(Q₁)(Q₂), —P(Q₁)(Q₂), —C(═O)(Q₁), —S(═O)(Q₁), —S(═O)₂(Q₁), —P(═O)(Q₁)(Q₂), or —P(═S)(Q₁)(Q₂),
b11, b14, and b19 are each independently 0, 1, 2, or 3,
b12, b15, and b18 are each independently 0, 1, 2, 3, or 4,
b13 and b16 are each independently 0, 1, or 2, and
b17 is 0 or 1,
wherein Q₁ to Q₃ are each independently hydrogen, deuterium, —F, —Cl, —Br, —I, a hydroxyl group, a cyano group, a nitro group, an amidino group, a hydrazino group, a hydrazono group, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₇-C₆₀ alkyl aryl group, a C₆-C₆₀ aryloxy group, a C₆-C₆₀ arylthio group, a C₁-C₆₀ heteroaryl group, a C₂-C₆₀ alkyl heteroaryl group, a C₁-C₆₀ heteroaryloxy group, a C₁-C₆₀ heteroarylthio group, a monovalent aromatic condensed polycyclic group, a monovalent aromatic condensed heteropolycyclic group, a monovalent non-aromatic condensed polycyclic group, a monovalent non-aromatic condensed heteropolycyclic group, a C₁-C₆₀ alkyl group substituted with at least one deuterium, —F, a cyano group, a C₁-C₆₀ alkyl group, a C₆-C₆₀ aryl group, or any combination thereof, or a C₆-C₆₀ aryl group substituted with at least one deuterium, —F, a cyano group, a C₁-C₆₀ alkyl group, a C₆-C₆₀ aryl group, or any combination thereof;
in Formula 1-1, a case where X₁₄ is *1, X₁₅ is *2, W₁₂ is *1, W₁₁ is *2, Y₁₅ is *3, Y₁₄ is *4, W₁₆ is *3, and W₁₅ is *4 is excluded.

2. The heterocyclic compound of claim 1, wherein
the heterocyclic compound is not represented by Formula X: 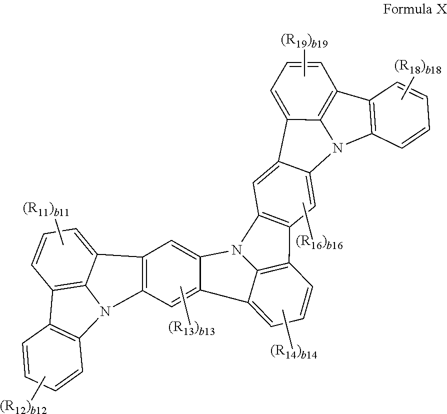
wherein, in Formula X, R₁₁ to R₁₄, R₁₆, R₁₈, R₁₉, b11 to b14, b16, b18, and b19 are respectively understood by referring to the descriptions of R₁₁ to R₁₄, R₁₆, R₁₈, R₁₉, b11 to b14, b16, b18, and b19 in claim 1.

3. The heterocyclic compound of claim 1, wherein
in Formula 1-1, W₁₁ is *1, W₁₂ is *2, W₁₅ is *3, and W₁₆ is *4;
in Formula 1-1, W₁₁ is *1, W₁₂ is *2, W₁₅ is *4, and W₁₆ is *3;
in Formula 1-1, W₁₁ is *2, W₁₁ is *1, W₁₅ is *3, and W₁₆ is *4;
in Formula 1-2, W₁₃ is *6, W₁₄ is *5, W₁₅ is *8, and W₁₆ is *7;
in Formula 1-2, W₁₃ is *6, W₁₄ is *5, W₁₅ is *7, and W₁₆ is *8;
in Formula 1-2, W₁₃ is *5, W₁₄ is *6, W₁₅ is *8, and W₁₆ is *7; or
in Formula 1-2, W₁₃ is *6, W₁₄ is *6, W₁₅ is *7, and W₁₆ is *8.

4. The heterocyclic compound of claim 1, wherein
R₁₁ to R₁₉ are each independently: hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, —SF₅, a C₁-C₂₀ alkyl group, a C₁-C₂₀ alkenyl group, a C₁-C₂₀ alkoxy group, or a C₁-C₂₀ alkylthio group;
a C₁-C₂₀ alkyl group, a C₁-C₂₀ alkenyl group, a C₁-C₂₀ alkoxy group, or a C₁-C₂₀ alkylthio group, each substituted with deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₁₀ alkyl group, a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a bicyclo[1.1.1]pentyl group, a bicyclo[2.1.1]hexyl group, a bicyclo[2.2.1]heptyl group, a bicyclo[2.2.2]octyl group, a (C₁-C₂₀ alkyl)cyclopentyl group, a (C₁-C₂₀ alkyl)cyclohexyl group, a (C₁-C₂₀ alkyl)cycloheptyl group, a (C₁-C₂₀ alkyl)cyclooctyl group, a (C₁-C₂₀ alkyl)adamantanyl group, a (C₁-C₂₀ alkyl)norbornanyl group, a (C₁-C₂₀ alkyl)norbornenyl group, a (C₁-C₂₀ alkyl)cyclopentenyl group, a (C₁-C₂₀ alkyl)cyclohexenyl group, a (C₁-C₂₀ alkyl)cycloheptenyl group, a (C₁-C₂₀ alkyl)bicyclo[1.1.1]pentyl group, a (C₁-C₂₀ alkyl)bicyclo[2.1.1]hexyl group, a (C₁-C₂₀ alkyl)bicyclo[2.2.1]heptyl group, a (C₁-C₂₀ alkyl)bicyclo[2.2.2]octyl group, a phenyl group, a (C₁-C₂₀ alkyl)phenyl group, a biphenyl group, a terphenyl group, a naphthyl group, a pyridinyl group, a pyrimidinyl group, or any combination thereof;
a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a bicyclo[1.1.1]pentyl group, a bicyclo[2.1.1]hexyl group, a bicyclo[2.2.1]heptyl group, a bicyclo[2.2.2]octyl group, a phenyl group, a (C₁-C₂₀ alkyl)phenyl group, a biphenyl group, a terphenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, an isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, an imidazopyrimidinyl group, an azacarbazolyl group, an azadibenzofuranyl group, or an azadibenzothiophenyl group, each unsubstituted or substituted with deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a deuterated C₂-C₂₀ alkyl group, a C₁-C₂₀ alkoxy group, a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a bicyclo[1.1.1]pentyl group, a bicyclo[2.1.1]hexyl group, a bicyclo[2.2.1]heptyl group, a bicyclo[2.2.2]octyl group, a (C₁-C₂₀ alkyl)cyclopentyl group, a (C₁-C₂₀ alkyl)cyclohexyl group, a (C₁-C₂₀ alkyl)cycloheptyl group, a (C₁-C₂₀ alkyl)cyclooctyl group, a (C₁-C₂₀ alkyl)adamantanyl group, a (C₁-C₂₀ alkyl)norbornanyl group, a (C₁-C₂₀ alkyl)norbornenyl group, a (C₁-C₂₀ alkyl)cyclopentenyl group, a (C₁-C₂₀ alkyl)cyclohexenyl group, a (C₁-C₂₀ alkyl)cycloheptenyl group, a (C₁-C₂₀ alkyl)bicyclo[1.1.1]pentyl group, a (C₁-C₂₀ alkyl)bicyclo[2.1.1]hexyl group, a (C₁-C₂₀ alkyl)bicyclo[2.2.1]heptyl group, a (C₁-C₂₀ alkyl)bicyclo[2.2.2]octyl group, a phenyl group, a (C₁-C₂₀ alkyl)phenyl group, a biphenyl group, a terphenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, an isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, an imidazopyrimidinyl group, an azacarbazolyl group, an azadibenzofuranyl group, an azadibenzothiophenyl group, or any combination thereof; or
—Si(Q₁)(Q₂)(Q₃), —Ge(Q₁)(Q₂)(Q₃), —C(Q₁)(Q₂)(Q₃), —B(Q₁)(Q₂), or —N(Q₁)(Q₂),
wherein Q₁ to Q₃ are each independently:
deuterium, —F, —CH₃, —CD₃, —CD₂H, —CDH₂, —CH₂CH₃, —CH₂CD₃, —CH₂CD₂H, —CH₂CDH₂, —CHDCH₃, —CHDCD₂H, —CHDCDH₂, —CHDCD₃, —CD₂CD₃, —CD₂CD₂H, —CD₂CDH₂, —CF₃, —CF₂H, —CFH₂, —CH₂CF₃, —CH₂CF₂H, —CH₂CFH₂, —CHFCH₃, —CHFCF₂H, —CHFCFH₂, —CHFCF₃, —CF₂CF₃, —CF₂CF₂H, or —CF₂CFH₂; or
an n-propyl group, an iso-propyl group, an n-butyl group, a sec-butyl group, an isobutyl group, a tert-butyl group, an n-pentyl group, a tert-pentyl group, a neopentyl group, an isopentyl group, a sec-pentyl group, a 3-pentyl group, a sec-isopentyl group, a phenyl group, a biphenyl group, or a naphthyl group, each unsubstituted or substituted with deuterium, —F, a C₁-C₁₀ alkyl group, a phenyl group, or any combination thereof.

5. The heterocyclic compound of claim 1, wherein the heterocyclic compound is represented by Formula 13: 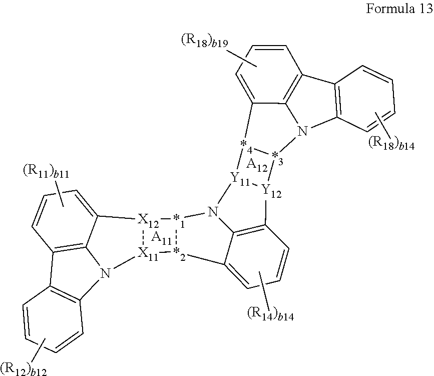
wherein, in Formula 13,
i)*1 is X₁₃, *2 is X₁₄, *3 is Y₁₃, and *4 is Y₁₄; ii)*1 is X₁₃, *2 is X₁₄, *3 is Y₁₄, and
*4 is Y₁₅; iii)*1 is X₁₃, *2 is X₁₄, *3 is Y₁₅, and *4 is Y₁₆; iv)*1 is X₁₄, *2 is X₁₅, *3 is Y₁₄, and *4 is Y₁₅; v)*1 is X₁₄, *2 is X₁₅, *3 is Y₁₅, and *4 is Y₁₆; or vi)*1 is X₁₅, *2 is X₁₆, *3 is Y₁₅, and *4 is Y₁₆.

6. The heterocyclic compound of claim 1, wherein the heterocyclic compound is represented by one of Formulae 13-1 to 13-5: 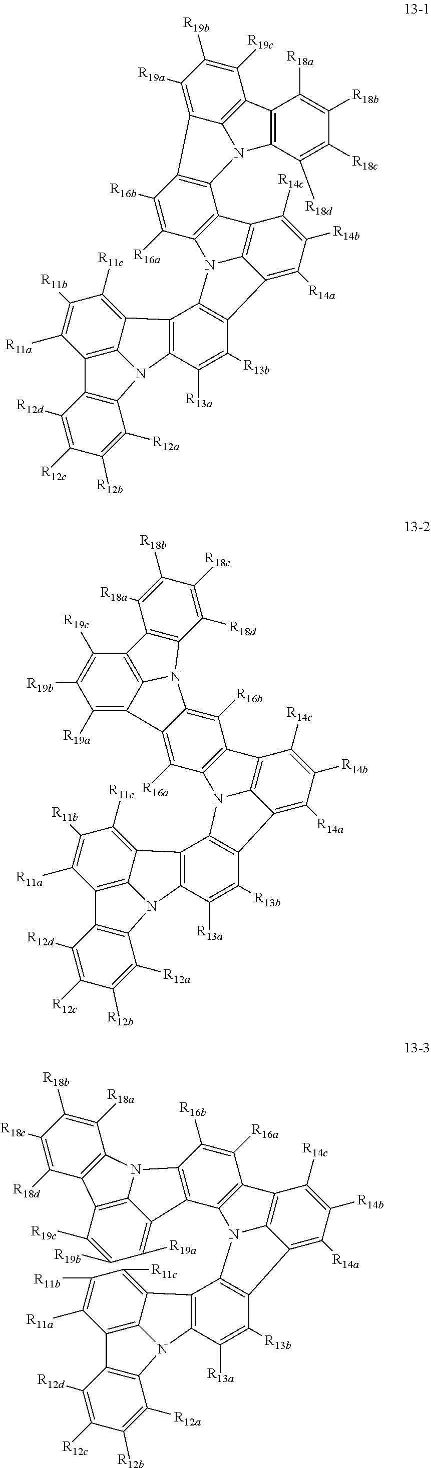 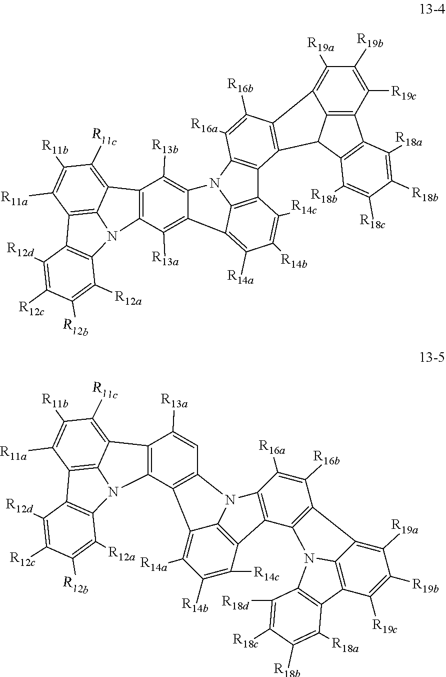
wherein, in Formulae 13-1 to 13-5,
R₁₁ₐ to R_{11c} are each understood by referring to the description of R₁₁ provided in claim 1,
R₁₂ₐ to R_{12d} are each understood by referring to the description of R₁₂ provided in claim 1,
R₁₃ₐ and R_{13b} are each understood by referring to the description of R₁₃ provided in claim 1,
R₁₄ₐ to R_{14c} are each understood by referring to the description of R₁₄ provided in claim 1,
R₁₅ₐ to R_{15d} are each understood by referring to the description of R₁₅ provided in claim 1,
R₁₆ₐ and R_{16b} are each understood by referring to the description of R₁₆ provided in claim 1,
R₁₇ₐ is understood by referring to the description of R₁₇ provided in claim 1,
R₁₈ₐ to R_{18d} are each understood by referring to the description of R₁₈ provided in claim 1, and
R₁₉ₐ to R_{19c} are each understood by referring to the description of R₁₉ provided in claim 1.

7. The heterocyclic compound of claim 6, R₁₁ₐ to R_{11c}, R₁₂ₐ to R₁₂d, R₁₃ₐ and R_{13b}, R₁₄ₐ to R_{14c}, R₁₅ₐ to R₁₅d, R₁₆ₐ and R_{16b}, R₁₇ₐ, R₁₈ₐ to R₁₈d, and R₁₉ₐ to R_{19c} are each independently hydrogen, deuterium, —F, —CH₃, —CD₃, —CD₂H, —CDH₂, —CF₃, —CF₂H, —CFH₂, a group represented by one of Formulae 9-1 to 9-39, a group represented by one of Formulae 9-1 to 9-39 in which at least one hydrogen is substituted with deuterium, a group represented by one of Formulae 9-1 to 9-39 in which at least one hydrogen is substituted with —F, a group represented by one of Formulae 10-1 to 10-130, a group represented by one of Formulae 10-1 to 10-130 in which at least one hydrogen is substituted with deuterium, a group represented by one of Formulae 10-1 to 10-130 in which at least one hydrogen is substituted with —F, a group represented by one of Formulae 10-359 to 10-380, a group represented by one of Formulae 10-359 to 10-380 in which at least one hydrogen is substituted with deuterium, a group represented by one of Formulae 10-359 to 10-380 in which at least one hydrogen is substituted with —F, —Si(Q₁)(Q₂)(Q₃), —Ge(Q₁)(Q₂)(Q₃), —C(Q₁)(Q₂)(Q₃), —B(Q₁)(Q₂), or —N(Q₁)(Q₂),
wherein Q₁ to Q₃ are each independently:
deuterium, —F, —CH₃, —CD₃, —CD₂H, —CDH₂, —CH₂CH₃, —CH₂CD₃, —CH₂CD₂H, —CH₂CDH₂, —CHDCH₃, —CHDCD₂H, —CHDCDH₂, —CHDCD₃, —CD₂CD₃, —CD₂CD₂H, —CD₂CDH₂, —CF₃, —CF₂H, —CFH₂, —CH₂CF₃, —CH₂CF₂H, —CH₂CFH₂, —CHFCH₃, —CHFCF₂H, —CHFCFH₂, —CHFCF₃, —CF₂CF₃, —CF₂CF₂H, or —CF₂CFH₂; or
an n-propyl group, an iso-propyl group, an n-butyl group, a sec-butyl group, an isobutyl group, a tert-butyl group, an n-pentyl group, a tert-pentyl group, a neopentyl group, an isopentyl group, a sec-pentyl group, a 3-pentyl group, a sec-isopentyl group, a phenyl group, a biphenyl group, or a naphthyl group, each unsubstituted or substituted with deuterium, —F, a C₁-C₁₀ alkyl group, a phenyl group, or any combination thereof:

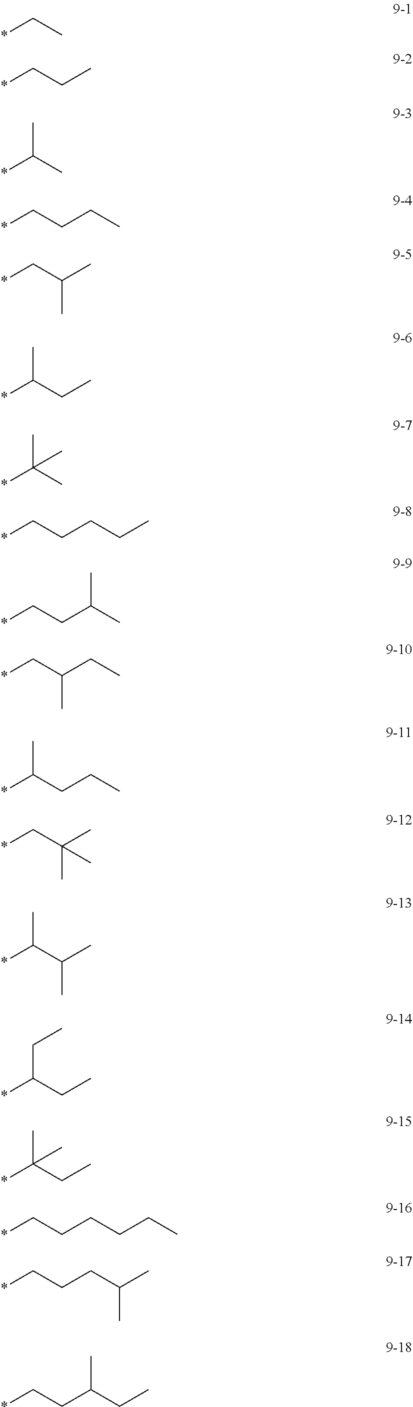

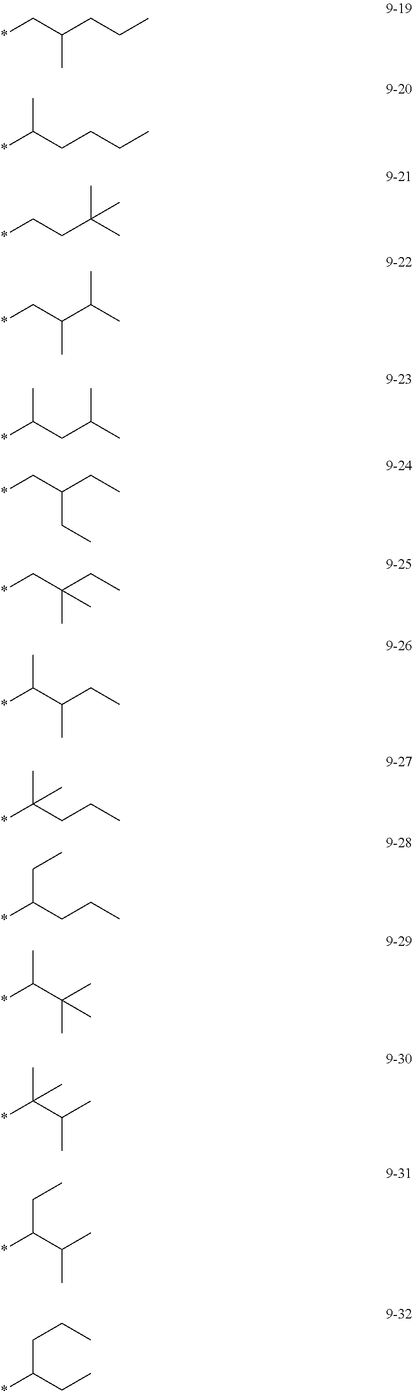

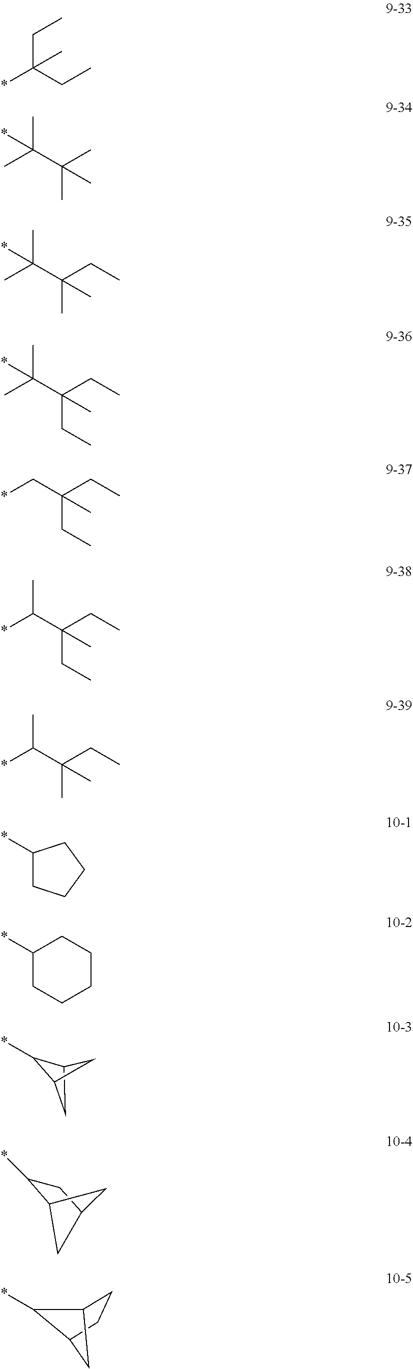

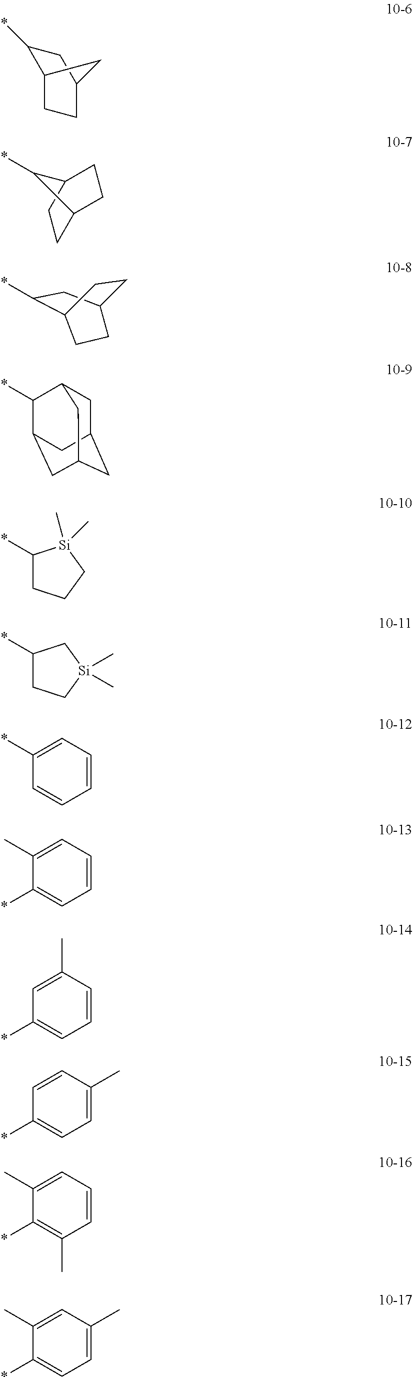

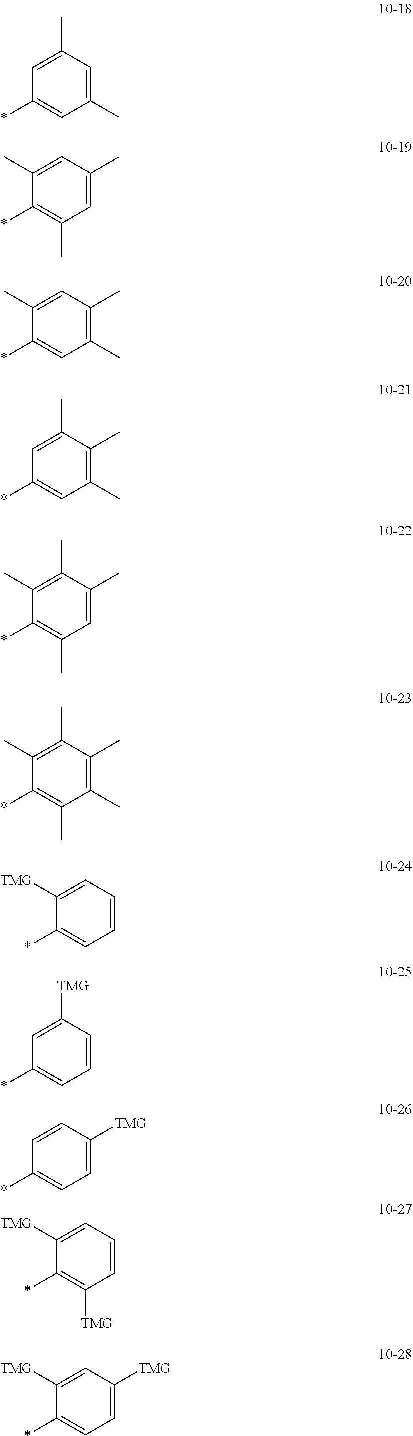

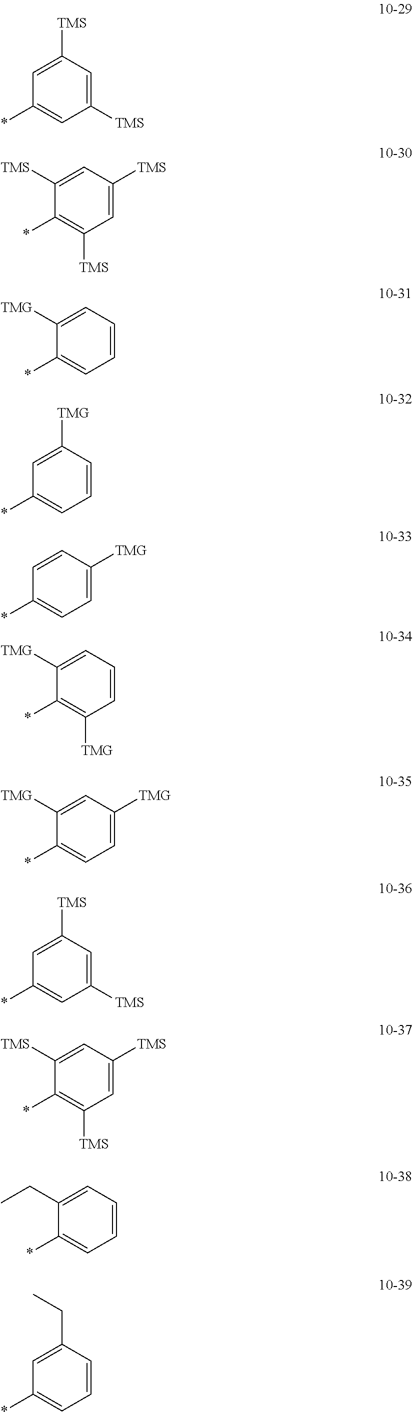

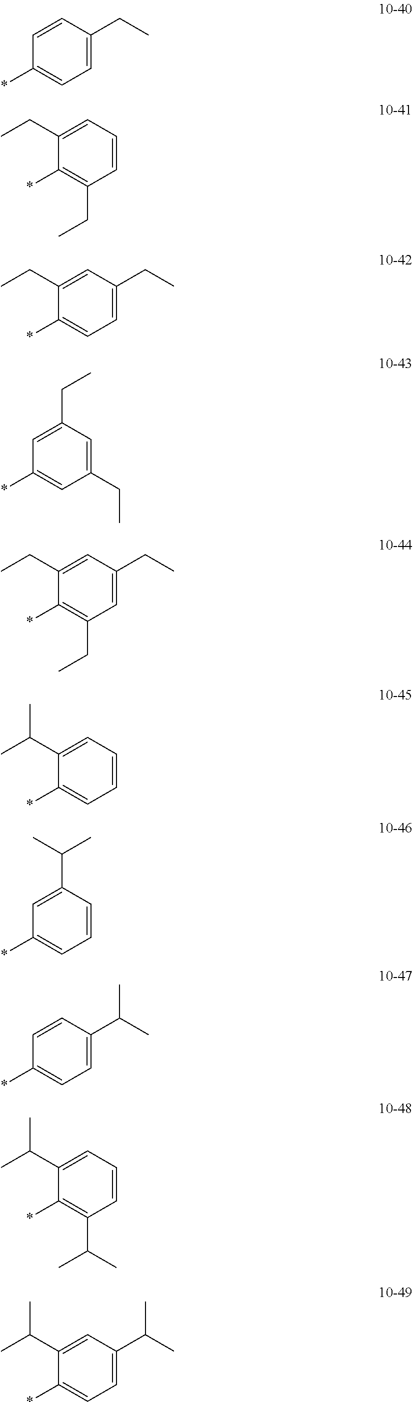

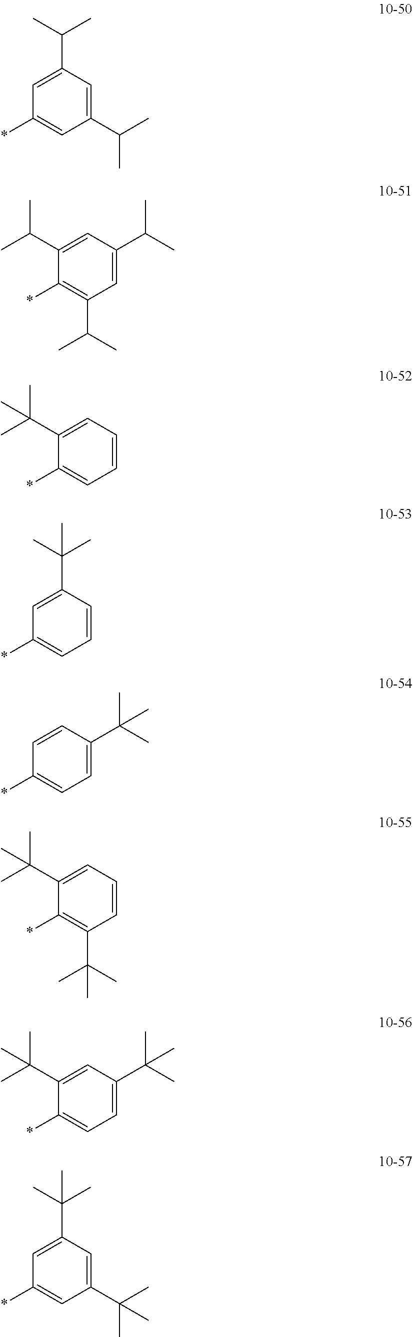

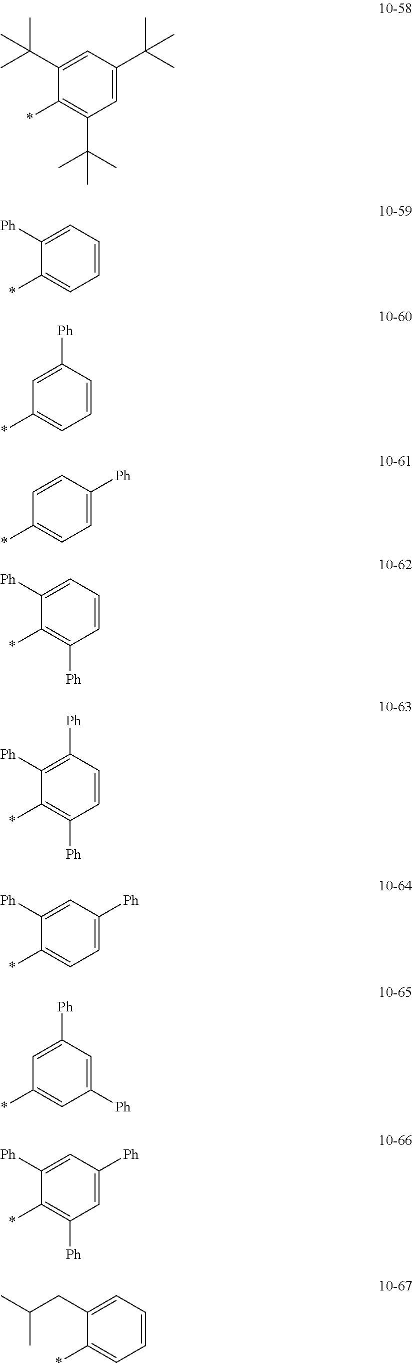

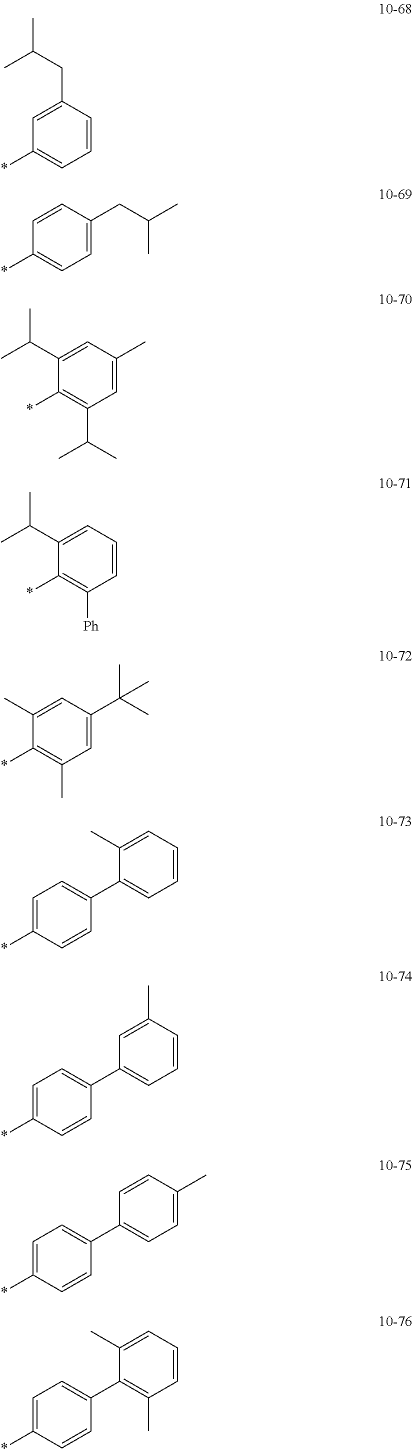

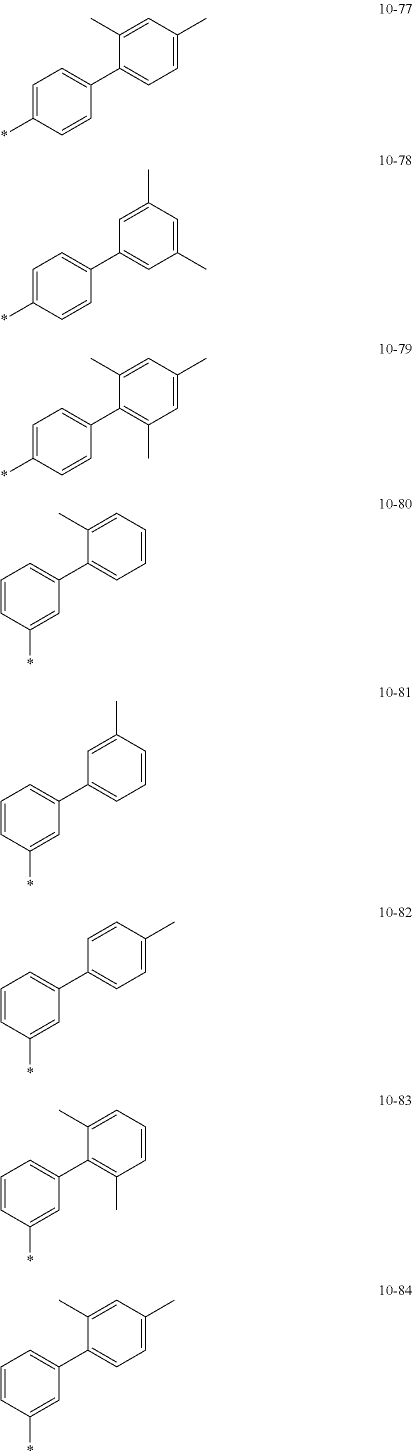

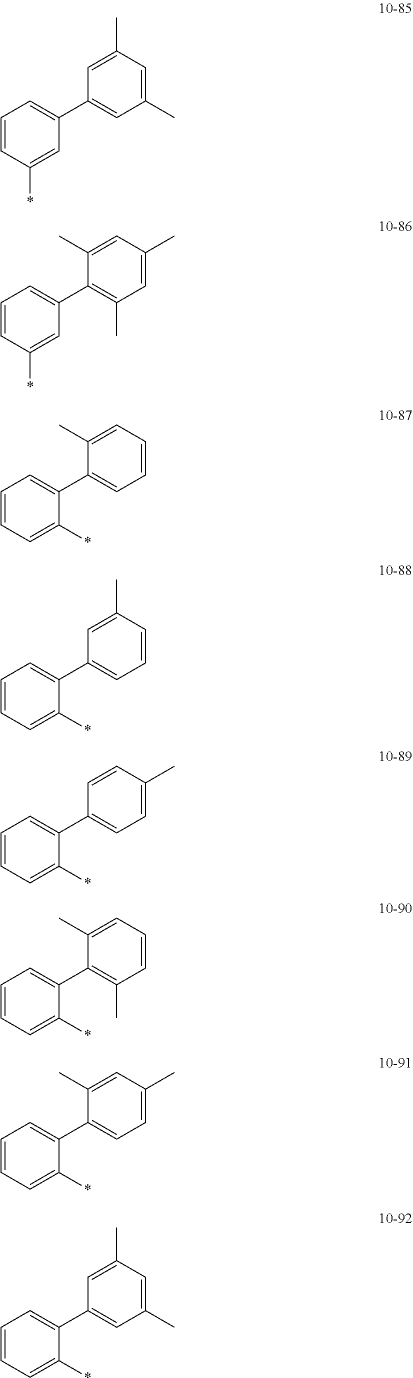

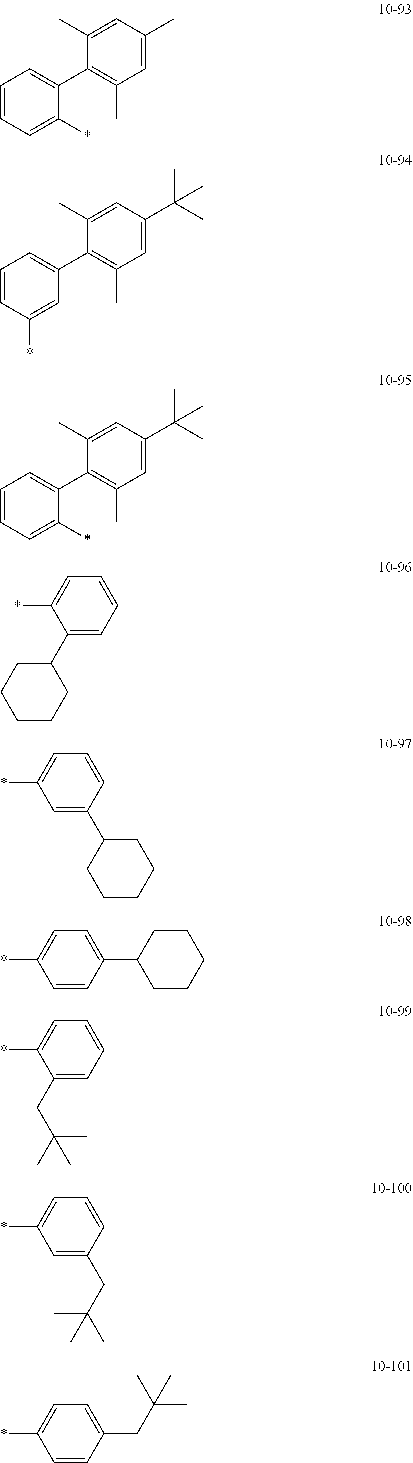

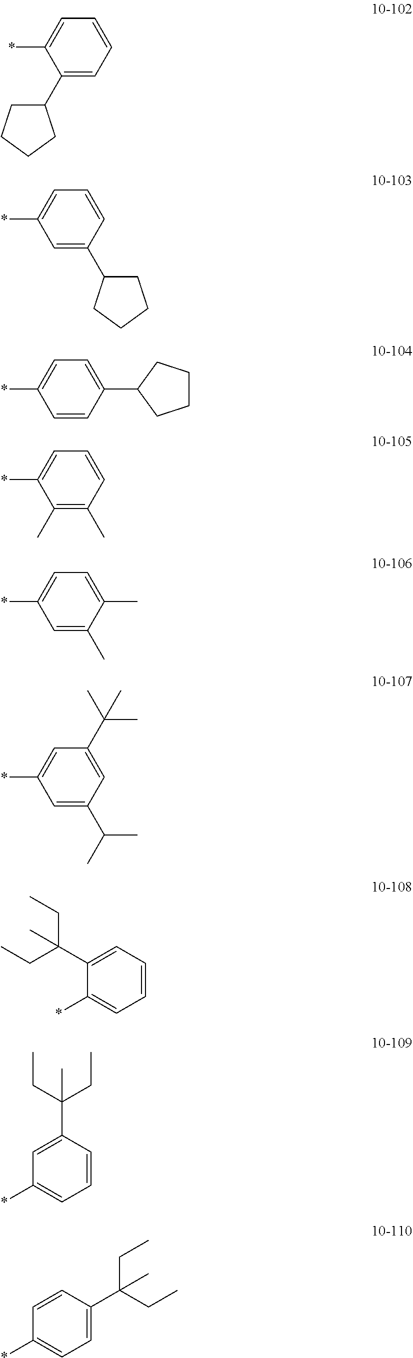

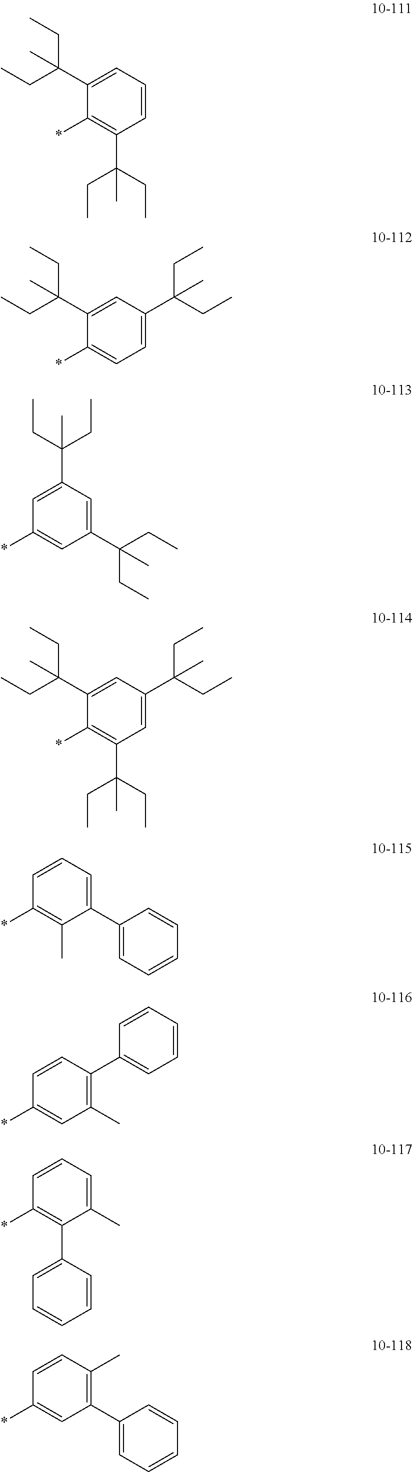

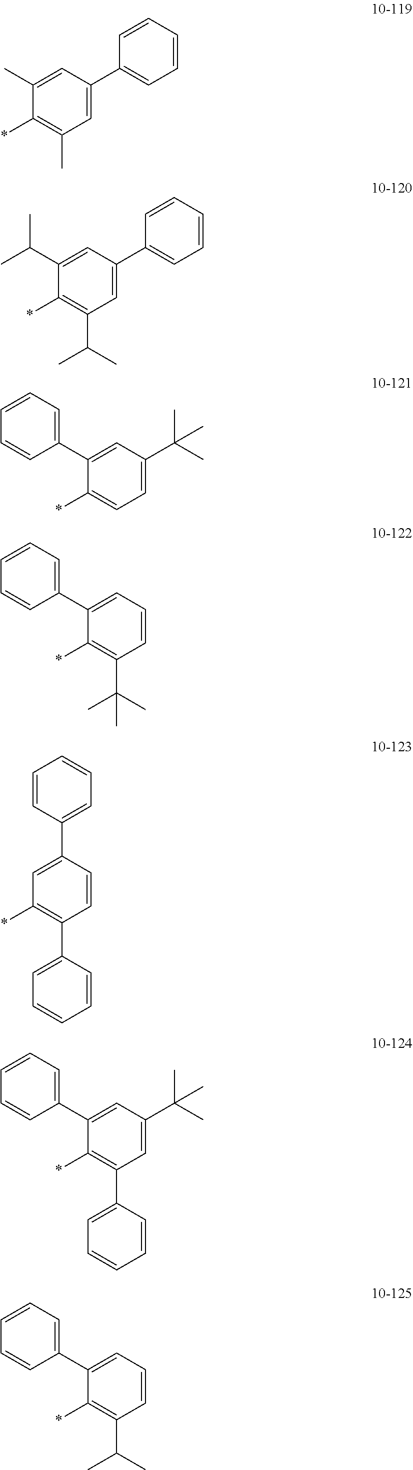

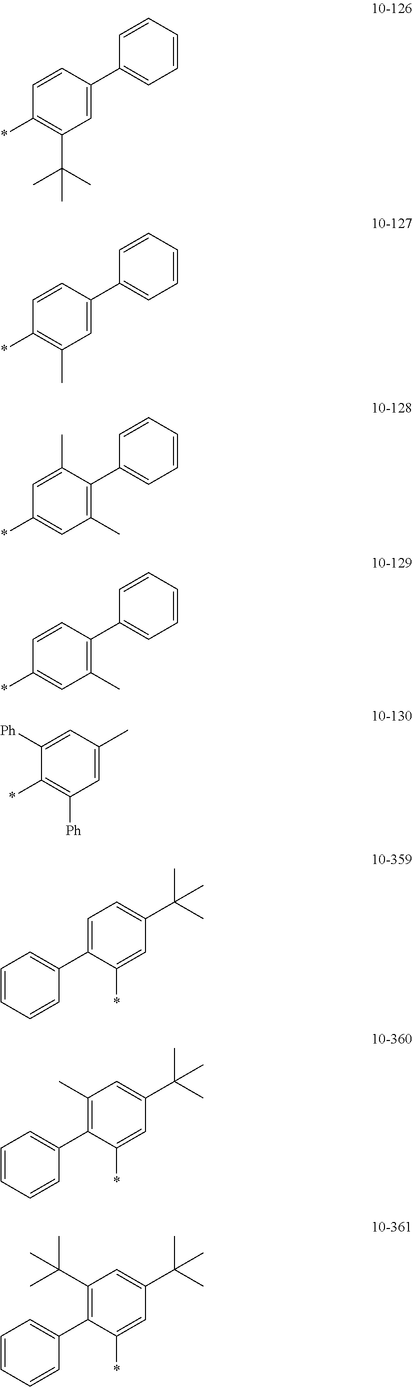

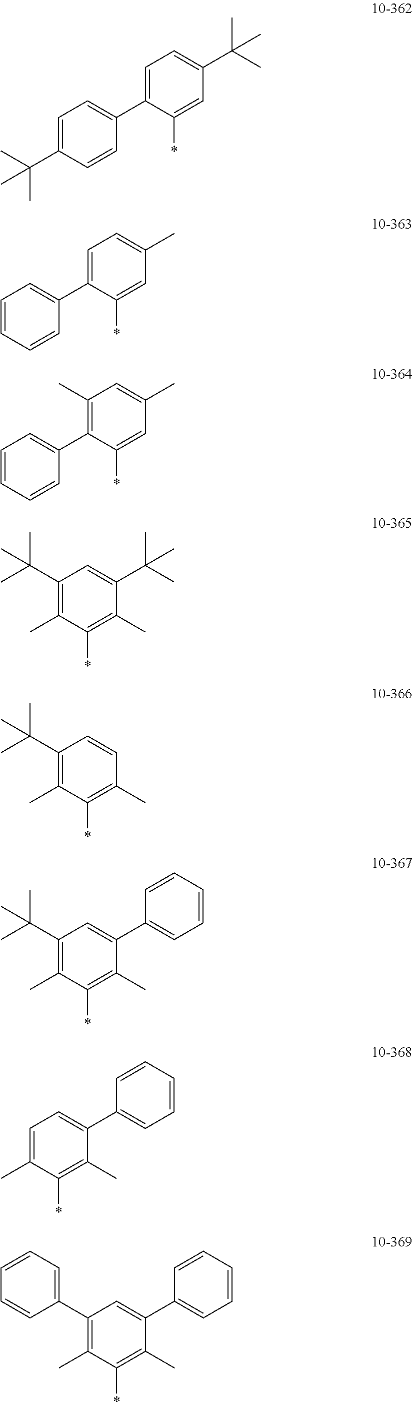

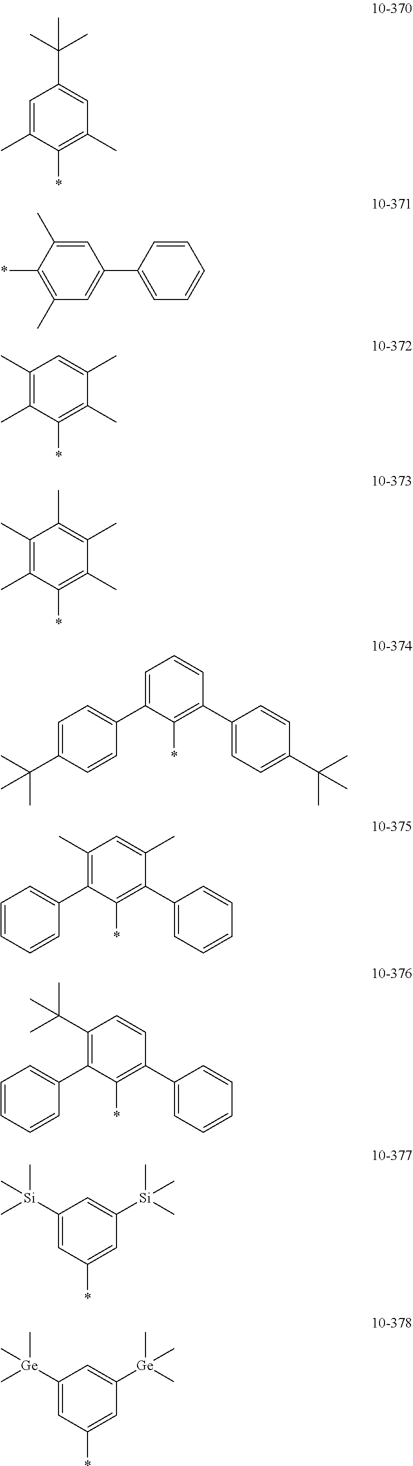

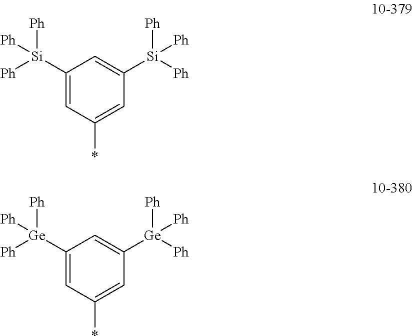

8. The heterocyclic compound of claim 6, wherein at least one of R_{11b}, R_{12c}, R_{14b}, R_{18b}, and R_{19b} in Formulae 11-2 and 11-5, at least one of R_{11b}, R_{12c}, R_{14b}, R_{18c}, and
R_{19b} in Formulae 12-1, 12-2, and 12-4, and at least one of R_{11b}, R_{12c}, R_{15c}, R_{18b}, and
R_{19b} in Formulae 14-1, 14-3, 14-4, 15-3, 16-3, 16-4, and 17-4 is —CH₃, —CD₃, —CD₂H, —CDH₂, —CF₃, —CF₂H, —CFH₂, a group represented by one of Formulae 9-1 to 9-39, a group represented by one of Formulae 9-1 to 9-39 in which at least one hydrogen is substituted with deuterium, a group represented by one of Formulae 9-1 to 9-39 in which at least one hydrogen is substituted with —F, a group represented by one of Formulae 10-1 to 10-130, a group represented by one of Formulae 10-1 to 10-130 in which at least one hydrogen is substituted with deuterium, a group represented by one of Formulae 10-1 to 10-130 in which at least one hydrogen is substituted with —F, a group represented by one of Formulae 10-359 to 10-380, a group represented by one of Formulae 10-359 to 10-380 in which at least one hydrogen is substituted with deuterium, a group represented by one of Formulae 10-359 to 10-380 in which at least one hydrogen is substituted with —F, —Si(Q₁)(Q₂)(Q₃), —Ge(Q₁)(Q₂)(Q₃), —C(Q₁)(Q₂)(Q₃), —B(Q₁)(Q₂), or —N(Q₁)(Q₂),
wherein Q₁ to Q₃ are each independently:
deuterium, —F, —CH₃, —CD₃, —CD₂H, —CDH₂, —CH₂CH₃, —CH₂CD₃, —CH₂CD₂H, —CH₂CDH₂, —CHDCH₃, —CHDCD₂H, —CHDCDH₂, —CHDCD₃, —CD₂CD₃, —CD₂CD₂H, —CD₂CDH₂, —CF₃, —CF₂H, —CFH₂, —CH₂CF₃, —CH₂CF₂H, —CH₂CFH₂, —CHFCH₃, —CHFCF₂H, —CHFCFH₂, —CHFCF₃, —CF₂CF₃, —CF₂CF₂H, or —CF₂CFH₂; or
an n-propyl group, an iso-propyl group, an n-butyl group, a sec-butyl group, an isobutyl group, a tert-butyl group, an n-pentyl group, a tert-pentyl group, a neopentyl group, an isopentyl group, a sec-pentyl group, a 3-pentyl group, a sec-isopentyl group, a phenyl group, a biphenyl group, or a naphthyl group, each unsubstituted or substituted with deuterium, —F, a C₁-C₁₀ alkyl group, a phenyl group, or any combination thereof.

9. The heterocyclic compound of claim 8, wherein the remaining substituents are each hydrogen.

10. The heterocyclic compound of claim 1, wherein the heterocyclic compound satisfies Conditions 1 to 4:
$$Δ {E}_{ST} > Δ {E}_{ST 2} + Δ {{E}^{′}}_{TT} \qquad Condition 1$$
$$0 eV < Δ {E}_{ST 2} + Δ {{E}^{′}}_{TT} \leq 1. eV \qquad Condition 2$$
$$0 eV < Δ {{E}^{′}}_{TT} \leq 0.3 eV \qquad Condition 3$$
$$Δ {E}_{ST 2} > 0 eV \qquad Condition 4$$
wherein, in Conditions 1 to 4,
ΔE_{ST} indicates a difference between a lowest excited singlet energy level calculated in an S₁ equilibrium structure of the heterocyclic compound and a lowest excited triplet energy level calculated in a T₁ equilibrium structure of the heterocyclic compound,
ΔE_{ST2} indicates a difference between a lowest excited singlet energy level calculated in an S₁ equilibrium structure of the heterocyclic compound and a second lowest excited triplet energy level calculated in a T₂ equilibrium structure of the heterocyclic compound, and
ΔE′_{TT} indicates a difference between a second lowest excited triplet energy level calculated in an T₂ equilibrium structure of the heterocyclic compound and a lowest excited triplet energy level calculated in a T₂ equilibrium structure of the heterocyclic compound.

11. An organic light-emitting device comprising: a first electrode;
a second electrode; and
an organic layer between the first electrode and the second electrode and comprising an emission layer,
wherein the organic layer comprises the heterocyclic compound of claim 1.

12. The organic light-emitting device of claim 11, wherein the emission layer comprises the heterocyclic compound.

13. The organic light-emitting device of claim 12, wherein the emission layer comprises a host and an emitter, the host is different from the emitter, and the emitter comprises the heterocyclic compound.

14. The organic light-emitting device of claim 13, wherein the emission layer emits blue light.

15. The organic light-emitting device of claim 13, wherein the emitter is a fluorescence emitter and/or a delayed fluorescence emitter.

16. The organic light-emitting device of claim 12, wherein the emission layer comprises a host, an emitter, and a sensitizer, the host, the emitter, and the sensitizer are different from one other, and the emitter comprises the heterocyclic compound.

17. The organic light-emitting device of claim 16, wherein the emission layer emits blue light.

18. The organic light-emitting device of claim 16, wherein the emitter is a fluorescence emitter and/or a delayed fluorescence emitter.

19. The organic light-emitting device of claim 16, wherein the heterocyclic compound satisfy Condition 5:
$$0 μ s < {T}_{decay} ( HC ) < 5 μ s \qquad Condition 5$$
wherein, in Condition 5,
T_{decay}(HC) indicates a decay time of the heterocyclic compound.

20. An electronic apparatus comprising the organic light-emitting device of claim 11.
